(19) United States
(12) Patent Application Publication
(10) Pub. No.: US 2007/0077553 A1
(43) Pub. Date: Apr. 5, 2007

(54) BIOINFORMATICALLY DETECTABLE GROUP OF NOVEL VACCINIA REGULATORY GENES AND USES THEREOF

(75) Inventor: Itzhak Bentwich, Kfar Daniel (IL)

Correspondence Address: ROSETTA-GENOMICS, 10 PLAUT-STREET SCIENCE PARK, P.O. BOX 2061, REHOVOT, 76706, IL

(73) Assignee: ROSETTA GENOMICS, Rehovot (IL)

(21) Appl. No.: 10/605,840

(22) Filed: Oct. 30, 2003

(51) Int. Cl. C12Q 1/70; C12Q 1/68; G06F 19/00; G01N 33/48; G01N 33/50; C07H 21/04

(52) U.S. Cl. 435/5; 435/6; 536/23.72; 702/20

(57) ABSTRACT

The present invention relates to a group of novel viral RNA regulatory genes, here identified as “viral genomic address messenger genes” or “VGAM genes”, and as “Viral genomic record” or “VGR genes”. VGAM genes selectively inhibit translation of known host target genes, and are believed to represent a novel pervasive viral attack mechanism. VGR genes encode an “operon”-like cluster of VGAM genes. VGAM and viral VGR genes may therefore be useful in diagnosing, preventing and treating viral disease. Several nucleic acid molecules are provided respectively encoding several VGAM genes, as are vectors and probes, both comprising the nucleic acid molecules, and methods and systems for detecting VGAM genes, and for counteracting their activity.

### CONTINUATION STATEMENT

This application is a continuation of U.S. patent application Ser. No. 10/604,943, filed 28 Aug. 2003, entitled “Bioinformatically Detectable Group of Novel Vaccinia Regulatory Genes and Uses of Thereof”, and is a continuation of U.S. Provisional Patent Application Ser. No. 60/441,241, filed 17 Jan. 2003, entitled “Bioinformatically Detectable Group of Novel Vaccinia Regulatory Genes and Uses of Thereof”, and is a continuation in part of U.S. patent application Ser. No. 10/604,942, filed 27 Aug. 2003, entitled “Bioinformatically Detectable Group of Novel Viral Regulatory Genes and Uses of Thereof”, and is a continuation in part of U.S. patent application Ser. No. 10/604,945, filed 27 Aug. 2003, entitled “Bioinformatically Detectable Group of Novel Viral Regulatory Genes and Uses of Thereof”, and is a continuation in part of U.S. Provisional Patent Application Ser. No. 60/457,788, filed 27 Mar. 2003, entitled “Bioinformatically Detectable Group of Novel Viral Regulatory Genes and Uses of Thereof”, and is a continuation in part of U.S. Provisional Patent Application Ser. No. 60/411,230, filed 17 Jan. 2003, entitled “Bioinformatically Detectable Group of Novel HIV Regulatory Genes and Uses of Thereof”, and is a continuation in part of U.S. patent application Ser. No. 10/310,188, filed 5 Dec. 2002, entitled “Bioinformatically Detectable Group of Novel Viral Regulatory Genes and Uses of Thereof”, and is a continuation in part of U.S. patent application Ser. No. 10/303,778, filed 26 Nov. 2002, entitled “Bioinformatically Detectable Group of Novel Viral Regulatory Genes and Uses of Thereof”, and is a continuation in part of U.S. patent application Ser. No. 10/604,944, filed 28 Aug. 2003, entitled “Bioinformatically Detectable Group of Novel HIV Regulatory Genes and Uses of Thereof”, and is a continuation in part of U.S. patent application Ser. No. 10/604,984, filed 29 Aug. 2003, entitled “Bioinformatically Detectable Group of Novel Viral Regulatory Genes and Uses of Thereof”, and is a continuation in part of U.S. patent application Ser. No. 10/605,838, filed 30 Oct. 2003, entitled “Bioinformatically Detectable Group of Novel HIV Regulatory Genes and Uses of Thereof”, the disclosures of which applications are all hereby incorporated by reference and claims priority therefrom.

### BACKGROUND OF INVENTION

1. Field of the Invention

The present invention relates to a group of bioinformatically detectable novel viral RNA regulatory genes, here identified as “viral genomic address messenger” or “VGAM” genes.

2. Description of Prior Art

Small RNAs are known to perform diverse cellular functions, including post-transcriptional gene expression regulation. The first two such RNA genes, Lin-4 and Let-7, were identified by genetic analysis of Caenorhabditis Elegans (Elegans) developmental timing, and were termed short temporal RNA (stRNA) (Wightman, B., Ha, I., Ruvkun, G., Cell 75, 855 (1993); Erdmann, V. A. et al., Nucleic Acids Res. 29, 189 (2001); Lee, R. C., Feinbaum, R. L., Ambros, V., Cell 75, 843 (1993); Reinhart, B. et al., Nature 403, 901 (2000)).

Lin-4 and Let-7 each transcribe a ˜22 nucleotide (nt) RNA, which acts a post transcriptional repressor of target mRNAs, by binding to elements in the 3″-untranslated region (UTR) of these target mRNAs, which are complimentary to the 22 nt sequence of Lin-4 and Let-7 respectively. While Lin-4 and Let-7 are expressed at different developmental stage, first larval stage and fourth larval stage respectively, both specify the temporal progression of cell fates, by triggering post-transcriptional control over other genes (Wightman, B., Ha, I., Ruvkun, G., Cell 75, 855 (1993); Slack et al., Mol. Cell 5, 659 (2000)). Let-7 as well as its temporal regulation have been demonstrated to be conserved in all major groups of bilaterally symmetrical animals, from nematodes, through flies to humans (Pasquinelli, A., et al. Nature 408, 86 (2000)).

The initial transcription product of Lin-4 and Let-7 is a ˜60-80 nt RNA, the nucleotide sequence of the first half of which is partially complimentary to that of its second half, therefore allowing this RNA to fold onto itself, forming a “hairpin structure”. The final gene product is a ˜22 nt RNA, which is “diced” from the above mentioned “hairpin structure”, by an enzyme called Dicer, which also apparently also mediates the complimentary binding of this ˜22 nt segment to a binding site in the 3″ UTR of its target gene. Recent studies have uncovered 93 new genes in this class, now referred to as micro RNA or miRNA genes, in genomes of Elegans, Drosophilea, and Human (Lagos-Quintana, M., Rauhut, R., Lendeckel, W., Tuschl, T., Science 294, 853 (2001); Lau, N. C., Lim, L. P., Weinstein, E. G., Bartel, D. P., Science 294, 858 (2001); Lee, R. C., Ambros, V., Science 294, 862 (2001). Like the well studied Lin-4 and Let-7, all newly found MIR genes produce a ˜60-80 nt RNA having a nucleotide sequence capable of forming a “hairpin structure”. Expressions of the precursor ˜60-80 nt RNA and of the resulting diced ˜22 nt RNA of most of these newly discovered MIR genes have been detected.

Based on the striking homology of the newly discovered MIR genes to their well-studied predecessors Lin-4 and Let-7, the new MIR genes are believed to have a similar basic function as that of Lin-4 and Let-7: modulation of target genes by complimentary binding to the UTR of these target genes, with special emphasis on modulation of developmental control processes. This is despite the fact that the above mentioned recent studies did not find target genes to which the newly discovered MIR genes complementarily bind. While existing evidence suggests that the number of regulatory RNA genes “may turn out to be very large, numbering in the hundreds or even thousands in each genome”, detecting such genes is challenging (Ruvkun G., “Perspective: Glimpses of a tiny RNA world”, Science 294, 779 (2001)).

The ability to detect novel RNA genes is limited by the methodologies used to detect such genes. All RNA genes identified so far either present a visibly discernable whole body phenotype, as do Lin-4 and Let-7 (Wightman et. al., Cell 75, 855 (1993); Reinhart et al., Nature 403, 901 (2000)), or produce significant enough quantities of RNA so as to be detected by the standard biochemical genomic techniques, as do the 93 recently detected miRNA genes. Since a limited number clones were sequenced by the researchers discovering these genes, 300 by Bartel and 100 by Tuschl (Bartel et. al., Science 294, 858 (2001); Tuschl et. al., Science 294, 853 (2001)), the RNA genes found can not be much rarer than 1% of all RNA genes. The recently detected miRNA genes therefore represent the more prevalent among the miRNA gene family.

Current methodology has therefore been unable to detect RNA genes which either do not present a visually discernable whole body phenotype, or are rare (e.g. rarer than 0.1% of all RNA genes), and therefore do not produce significant enough quantities of RNA so as to be detected by standard biochemical technique. To date, mRNA have not been detected in viruses.

### SUMMARY OF INVENTION

The present invention relates to a novel group of bioinformatically detectable, viral regulatory RNA genes, which repress expression of host target host genes, by means of complementary hybridization to binding sites in untranslated regions of these host target host genes. It is believed that this novel group of viral genes represent a pervasive viral mechanism of attacking hosts, and that therefore knowledge of this novel group of viral genes may be useful in preventing and treating viral diseases.

In various preferred embodiments, the present invention seeks to provide improved method and system for detection and prevention of viral disease, which is mediated by this group of novel viral genes.

Accordingly, the invention provides several substantially pure nucleic acids (e.g., genomic nucleic acid, cDNA or synthetic nucleic acid) each encoding a novel viral gene of the VGAM group of gene, vectors comprising the nucleic acids, probes comprising the nucleic acids, a method and system for selectively modulating translation of known “target” genes utilizing the vectors, and a method and system for detecting expression of known “target” genes utilizing the probe.

By “substantially pure nucleic acid” is meant nucleic acid that is free of the genes which, in the naturally-occurring genome of the organism from which the nucleic acid of the invention is derived, flank the genes discovered and isolated by the present invention. The term therefore includes, for example, a recombinant nucleic acid which is incorporated into a vector, into an autonomously replicating plasmid or virus, or into the genomic nucleic acid of a prokaryote or eukaryote at a site other than its natural site; or which exists as a separate molecule (e.g., a cDNA or a genomic or cDNA fragment produced by PCR or restriction endonuclease digestion) independent of other sequences. It also includes a recombinant nucleic acid which is part of a hybrid gene encoding additional polypeptide sequence.

“Inhibiting translation” is defined as the ability to prevent synthesis of a specific protein encoded by a respective gene, by means of inhibiting the translation of the mRNA of this gene. “Translation inhibitor site” is defined as the minimal nucleic acid sequence sufficient to inhibit translation.

There is thus provided in accordance with a preferred embodiment of the present invention a bioinformatically detectable novel viral gene encoding substantially pure nucleic acid wherein: RNA encoded by the bioinformatically detectable novel viral gene is about 18 to about 24 nucleotides in length, and originates from an RNA precursor, which RNA precursor is about 50 to about 120 nucleotides in length, a nucleotide sequence of a first half of the RNA precursor is a partial inversed-reversed sequence of a nucleotide sequence of a second half thereof, a nucleotide sequence of the RNA encoded by the novel viral gene is a partial inversed-reversed sequence of a nucleotide sequence of a binding site associated with at least one host target gene, and a function of the novel viral gene is bioinformatically deducible.

There is further provided in accordance with another preferred embodiment of the present invention a method for anti-viral treatment comprising neutralizing said RNA.

Further in accordance with a preferred embodiment of the present invention the neutralizing comprises: synthesizing a complementary nucleic acid molecule, a nucleic sequence of which complementary nucleic acid molecule is a partial inversed-reversed sequence of said RNA, and transfecting host cells with the complementary nucleic acid molecule, thereby complementarily binding said RNA.

Further in accordance with a preferred embodiment of the present invention the neutralizing comprises immunologically neutralizing.

There is still further provided in accordance with another preferred embodiment of the present invention a bioinformatically detectable novel viral gene encoding substantially pure nucleic acid wherein: RNA encoded by the bioinformatically detectable novel viral gene includes a plurality of RNA sections, each of the RNA sections being about 50 to about 120 nucleotides in length, and including an RNA segment, which RNA segment is about 18 to about 24 nucleotides in length, a nucleotide sequence of a first half of each of the RNA sections encoded by the novel viral gene is a partial inversed-reversed sequence of nucleotide sequence of a second half thereof, a nucleotide sequence of each of the RNA segments encoded by the novel viral gene is a partial inversed-reversed sequence of the nucleotide sequence of a binding site associated with at least one target host gene, and a function of the novel viral gene is bioinformatically deducible from the following data elements: the nucleotide sequence of the RNA encoded by the novel viral gene, a nucleotide sequence of the at least one target host gene, and function of the at least one target host gene.

Further in accordance with a preferred embodiment of the present invention the function of the novel viral gene is bioinformatically deducible from the following data elements: the nucleotide sequence of the RNA encoded by the bioinformatically detectable novel viral gene, a nucleotide sequence of the at least one target host gene, and a function of the at least one target host gene.

Still further in accordance with a preferred embodiment of the present invention the RNA encoded by the novel viral gene complementarily binds the binding site associated with the at least one target host gene, thereby modulating expression of the at least one target host gene.

Additionally in accordance with a preferred embodiment of the present invention the binding site associated with at least one target host gene is located in an untranslated region of RNA encoded by the at least one target host gene.

Moreover in accordance with a preferred embodiment of the present invention the function of the novel viral gene is selective inhibition of translation of the at least one target host gene, which selective inhibition includes complementary hybridization of the RNA encoded by the novel viral gene to the binding site.

Further in accordance with a preferred embodiment of the present invention the invention includes a vector including the DNA.

Still further in accordance with a preferred embodiment of the present invention the invention includes a method of selectively inhibiting translation of at least one gene, including introducing the vector.

Moreover in accordance with a preferred embodiment of the present invention the introducing includes utilizing RNAi pathway.

Additionally in accordance with a preferred embodiment of the present invention the invention includes a gene expression inhibition system including: the vector, and a vector inserter, functional to insert the vector into a cell, thereby selectively inhibiting translation of at least one gene.

Further in accordance with a preferred embodiment of the present invention the invention includes a probe including the DNA.

Still further in accordance with a preferred embodiment of the present invention the invention includes a method of selectively detecting expression of at least one gene, including using the probe.

Additionally in accordance with a preferred embodiment of the present invention the invention includes a gene expression detection system including: the probe, and a gene expression detector functional to selectively detect expression of at least one gene.

Further in accordance with a preferred embodiment of the present invention the invention includes an anti-viral substance capable of neutralizing the RNA.

Still further in accordance with a preferred embodiment of the present invention the neutralizing includes complementarily binding the RNA.

Additionally in accordance with a preferred embodiment of the present invention the neutralizing includes immunologically neutralizing.

Moreover in accordance with a preferred embodiment of the present invention the invention includes a method for anti-viral treatment including neutralizing the RNA.

Further in accordance with a preferred embodiment of the present invention the neutralizing includes: synthesizing a complementary nucleic acid molecule, a nucleic sequence of which complementary nucleic acid molecule is a partial inversed-reversed sequence of the RNA, and transfecting host cells with the complementary nucleic acid molecule, thereby complementarily binding the RNA.

Still further in accordance with a preferred embodiment of the present invention the neutralizing includes immunologically neutralizing.

### BRIEF DESCRIPTION OF DRAWINGS

FIG. 1 is a simplified diagram illustrating a mode by which viral genes of a novel group of viral genes of the present invention, modulate expression of known host target genes;

FIG. 2 is a simplified block diagram illustrating a bioinformatic gene detection system capable of detecting genes of the novel group of genes of the present invention, which system is constructed and operative in accordance with a preferred embodiment of the present invention;

FIG. 3 is a simplified flowchart illustrating operation of a mechanism for training of a computer system to recognize the novel genes of the present invention, which mechanism is constructed and operative in accordance with a preferred embodiment of the present invention;

FIG. 4A is a simplified block diagram of a non-coding genomic sequence detector constructed and operative in accordance with a preferred embodiment of the present invention;

FIG. 4B is a simplified flowchart illustrating operation of a non-coding genomic sequence detector constructed and operative in accordance with a preferred embodiment of the present invention;

FIG. 5A is a simplified block diagram of a hairpin detector constructed and operative in accordance with a preferred embodiment of the present invention;

FIG. 5B is a simplified flowchart illustrating operation of a hairpin detector constructed and operative in accordance with a preferred embodiment of the present invention;

FIG. 6A is a simplified block diagram of a dicer-cut location detector constructed and operative in accordance with a preferred embodiment of the present invention;

FIG. 6B is a simplified flowchart illustrating training of a dicer-cut location detector constructed and operative in accordance with a preferred embodiment of the present invention;

FIG. 7A is a simplified block diagram of a target-gene binding-site detector constructed and operative in accordance with a preferred embodiment of the present invention;

FIG. 7B is a simplified flowchart illustrating operation of a target-gene binding-site detector constructed and operative in accordance with a preferred embodiment of the present invention;

FIG. 8 is a simplified flowchart illustrating operation of a function & utility analyzer constructed and operative in accordance with a preferred embodiment of the present invention;

FIG. 9 is a simplified diagram describing a novel bioinformatically detected group of regulatory viral genes, referred to here as Viral Genomic Record (VGR) genes, each of which encodes an “operon-like” cluster of novel viral mRNA-like genes, which in turn modulates expression of a plurality of host target genes;

FIG. 10 is a block diagram illustrating different utilities of genes of a novel group of genes, and operons of a novel group of operons, both of the present invention;

FIGS. 11A and 11B are simplified diagrams, which when taken together illustrate a mode of gene therapy applicable to genes of the novel group of genes of the present invention;

FIG. 12A is an annotated sequence of EST72223 comprising novel gene GAM24 detected by the gene detection system of the present invention;

FIGS. 12B and 12C are pictures of laboratory results, which when taken together demonstrate laboratory confirmation of expression of the bioinformatically detected novel gene GAM24 of FIG. 12A;

FIG. 12D provides pictures of laboratory results, which when taken together demonstrate further laboratory confirmation of expression of the bioinformatically detected novel gene GAM24 of FIG. 12A;

FIG. 13A is an annotated sequence of an EST7929020 comprising novel genes GAM23 and GAM25 detected by the gene detection system of the present invention;

FIG. 13B is a picture of laboratory results, which confirm expression of bioinformatically detected novel genes GAM23 and GAM25 of FIG. 13A;

FIG. 13C is a picture of laboratory results, which confirm endogenous expression of bioinformatically detected novel gene GAM25 of FIG. 15A;

FIG. 14A is an annotated sequence of an EST1388749 comprising novel gene GAM26 detected by the gene detection system of the present invention;

FIG. 14B is a picture of laboratory results, which confirm expression of the bioinformatically detected novel gene GAM26 of FIG. 14A;

### BRIEF DESCRIPTION OF SEQUENCES

A Sequence Listing of genomic sequences of the present invention designated SEQ ID:1 through SEQ ID:3750 is attached to this application. The genomic listing comprises the following nucleotide sequences: Genomic sequences designated SEQ ID:1 through SEQ ID:349 are nucleotide sequences of 349 gene precursors of respective novel genes of the present invention; Genomic sequences designated SEQ ID:350 through SEQ ID:698 are nucleotide sequences of 349 genes of the present invention; and Genomic sequences designated SEQ ID:699 through SEQ ID:3750 are nucleotide sequences of 3052 host target binding sites.

### DETAILED DESCRIPTION

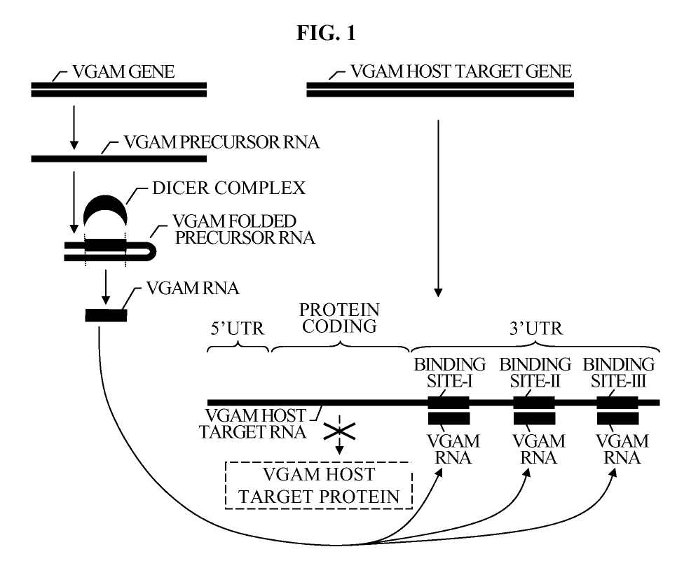

Reference is now made to FIG. 1 which is a simplified diagram illustrating a mode by which genes of a novel group of viral genes of the present invention, modulate expression of known host target genes.

The novel genes of the present invention are viral micro RNA (miRNA)-like, regulatory RNA genes, modulating expression of known host target genes. This mode of modulation is common to other known miRNA genes, as described hereinabove with reference to the background of the invention section.

VGAM GENE is a viral gene contained in the virus genome and TARGET GENE is a human gene contained in the DNA of the human genome.

VGAM GENE encodes a VGAM PRECURSOR RNA. However, similar to other miRNA genes, and unlike most ordinary genes, its RNA, VGAM PRECURSOR RNA, does not encode a protein.

VGAM PRECURSOR RNA folds onto itself, forming VGAM FOLDED PRECURSOR RNA. As FIG. 1 illustrates, VGAM FOLDED PRECURSOR RNA forms a “hairpin structure” folding onto itself. As is well known in the art, this “hairpin structure” is typical genes of the miRNA genes, and is due to the fact that nucleotide sequence of the first half of the RNA of a gene in this group is an accurate or partial inversed-reversed sequence of the nucleotide sequence of its second half. By “inversed-reversed” is meant a sequence which is reversed and wherein each nucleotide is replaced by a complimentary nucleotide, as is well known in the art (e.g. ATGGC is the inversed-reversed sequence of GCCAT).

An enzyme complex, designated DICER COMPLEX, “dices” the VGAM FOLDED PRECURSOR RNA into a single stranded RNA segment, about 22 nucleotides long, designated VGAM RNA. As is known in the art, “dicing” of the hairpin structured RNA precursor into shorter RNA segments about 22 nucleotides long by a Dicer type enzyme is catalyzed by an enzyme complex comprising an enzyme called Dicer together with other necessary proteins.

VGAM HOST TARGET GENE encodes a corresponding messenger RNA, designated VGAM HOST TARGET RNA. This VGAM HOST TARGET RNA comprises three regions: a 5″ untranslated region, a protein coding region and a 3″ untranslated region, designated 5″UTR, PROTEIN CODING and 3″UTR respectively.

VGAM RNA binds complementarily a BINDING SITE, located on the 3″UTR segment of TARGET RNA. This complementarily binding is due to the fact that the nucleotide sequence of VGAM RNA is an accurate or partial inversed-reversed sequence of the nucleotide sequence of BINDING SITE.

The complimentary binding of VGAM RNA to BINDING SITE inhibits translation of VGAM HOST TARGET RNA into VGAM HOST TARGET PROTEIN. VGAM HOST TARGET PROTEIN is therefore outlined by a broken line.

It is appreciated by one skilled in the art that the mode of transcriptional inhibition illustrated by FIG. 1 with specific reference to VGAM genes of the present invention, is in fact common to all other miRNA genes. A specific complimentary binding site has been demonstrated only for Lin-4 and Let-7. All the other 93 newly discovered miRNA genes are also believed by those skilled in the art to modulate expression of other genes by complimentary binding, although specific complimentary binding sites for these genes have not yet been found (Ruvkun G., “Perspective: Glimpses of a tiny RNA world”, Science 294, 779 (2001)). The present invention discloses a novel group of viral genes, the VGAM genes, belonging to the miRNA genes group, and for which a specific an complimentary binding has been determined.

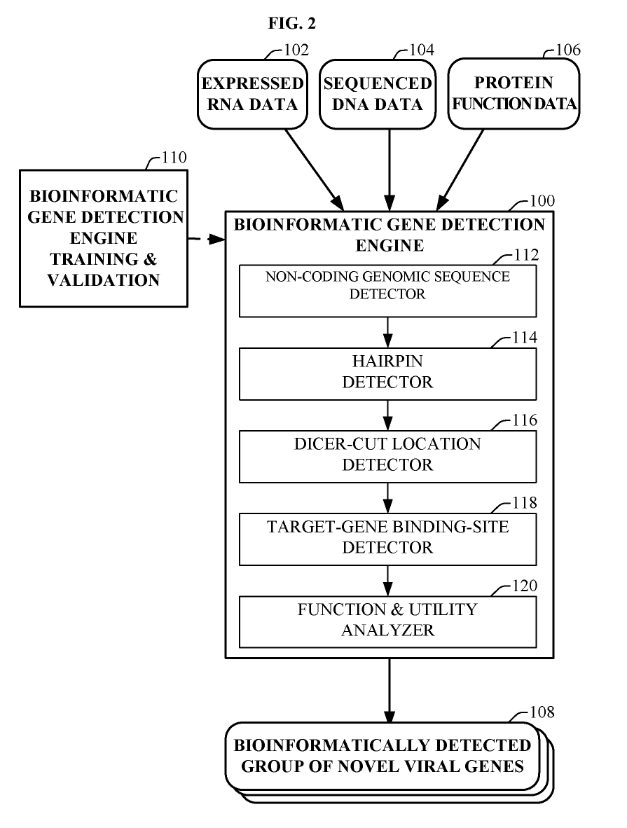

Reference is now made to FIG. 2 which is a simplified block diagram illustrating a bioinformatic gene detection system capable of detecting genes of the novel group of genes of the present invention, which system is constructed and operative in accordance with a preferred embodiment of the present invention.

A centerpiece of the present invention is a bioinformatic gene detection engine 100, which is a preferred implementation of a mechanism capable of bioinformatically detecting genes of the novel group of genes of the present invention. The function of the bioinformatic gene detection engine 100 is as follows: it receives three types of input, expressed RNA data 102, sequenced DNA data 104, and protein function data 106, performs a complex process of analysis of this data as elaborated below, and based on this analysis produces output of a bioinformatically detected group of novel genes designated 108.

Expressed RNA data 102 comprises published expressed sequence tags (EST) data, published mRNA data, as well as other sources of published RNA data. Sequenced DNA data 104 comprises alphanumeric data describing sequenced genomic data, which preferably includes annotation data such as location of known protein coding regions relative to the sequenced data. Protein function data 106 comprises scientific publications reporting studies which elucidated physiological function known proteins, and their connection, involvement and possible utility in treatment and diagnosis of various diseases. Expressed RNA data 102, sequenced DNA data 104 may preferably be obtained from data published by the National Center for Bioinformatics (NCBI) at the National Institute of Health (NIH), as well as from various other published data sources. Protein function data 106 may preferably be obtained from any one of numerous relevant published data sources, such as the Online Mendelian Inherited Disease In Man (OMIM) database developed by John Hopkins University, and also published by NCBI.

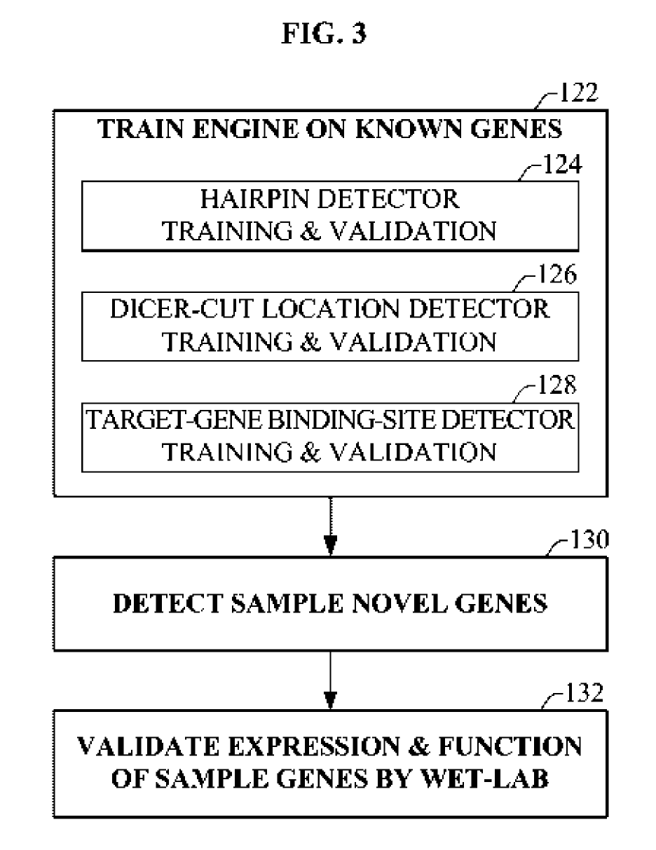

Prior to actual detection of bioinformatically detected novel genes 108 by the bioinformatic gene detection engine 100, a process of bioinformatic gene detection engine training & validation designated 110 takes place. This process uses the known miRNA genes as a training set (some 200 such genes have been found to date using biological laboratory means), to train the bioinformatic gene detection engine 100 to bioinformatically recognize mRNA-like genes, and their respective potential target binding sites. Bioinformatic gene detection engine training & validation 110 is further describe hereinbelow with reference to FIG. 3.

The bioinformatic gene detection engine 100 comprises several modules which are preferably activated sequentially, and are described as follows:

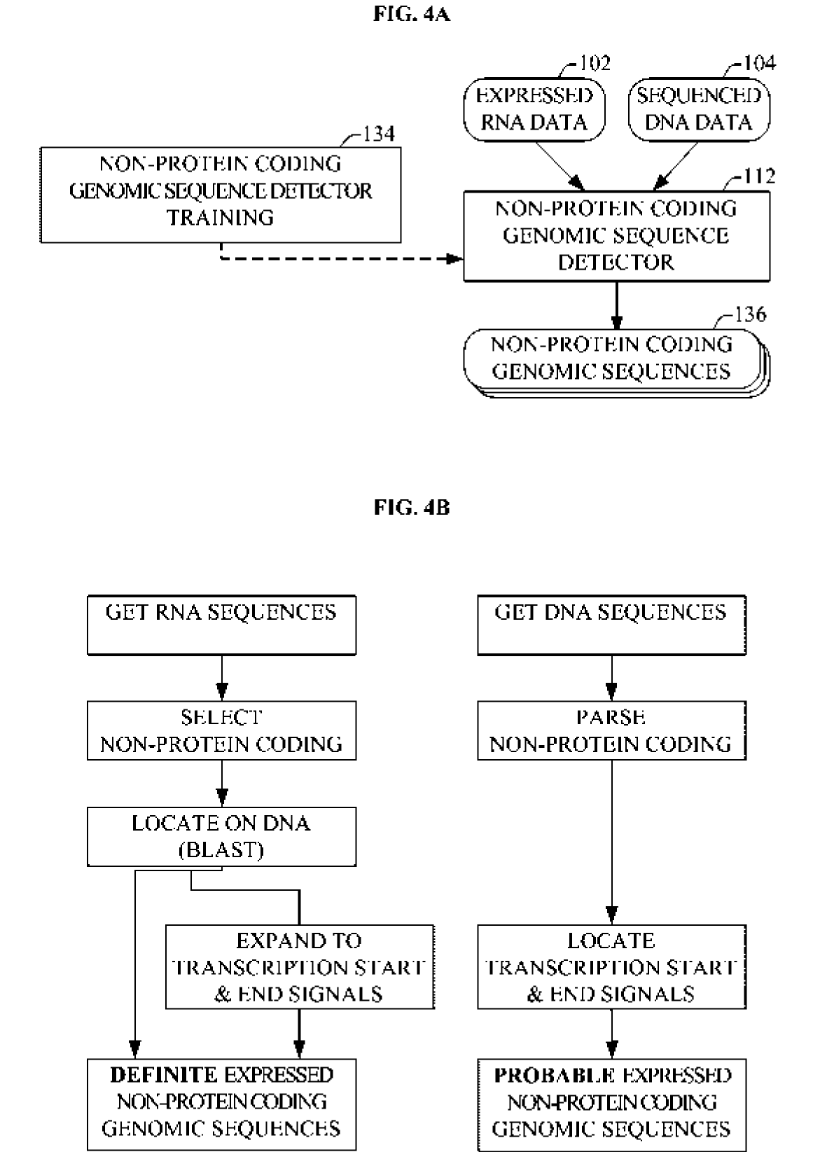

A non-coding genomic sequence detector 112 operative to bioinformatically detect non-protein coding genomic sequences. The non-coding genomic sequence detector 112 is further described hereinbelow with reference to FIGS. 4A and 4B.

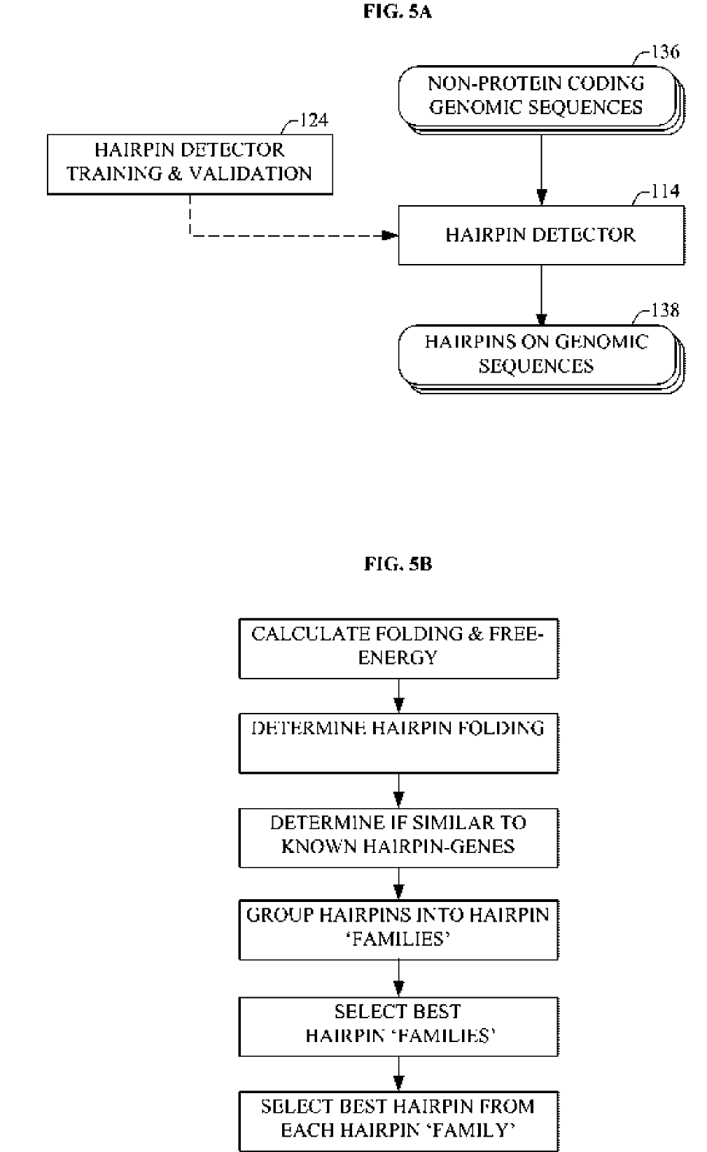

A hairpin detector 114 operative to bioinformatically detect genomic “hairpin-shaped” sequences, similar to VGAM FOLDED PRECURSOR of FIG. 1. The hairpin detector 114 is further described hereinbelow with reference to FIGS. 5A and 5B.

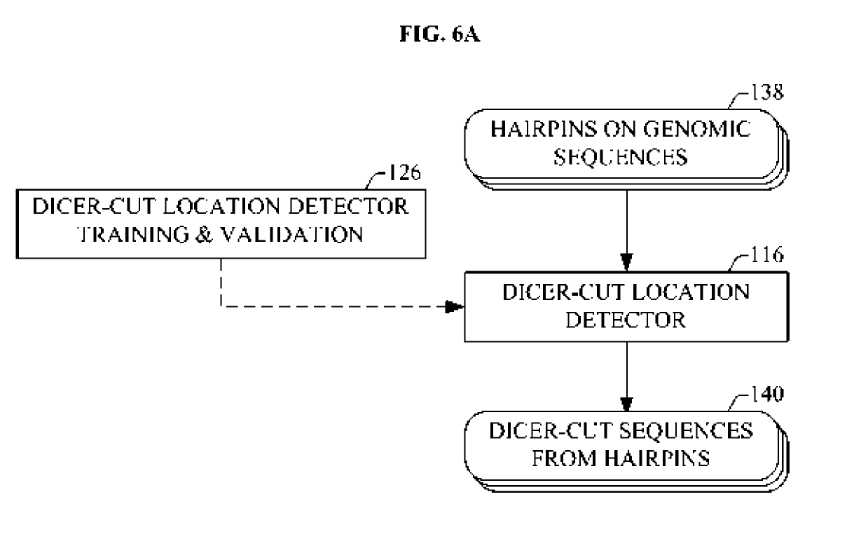

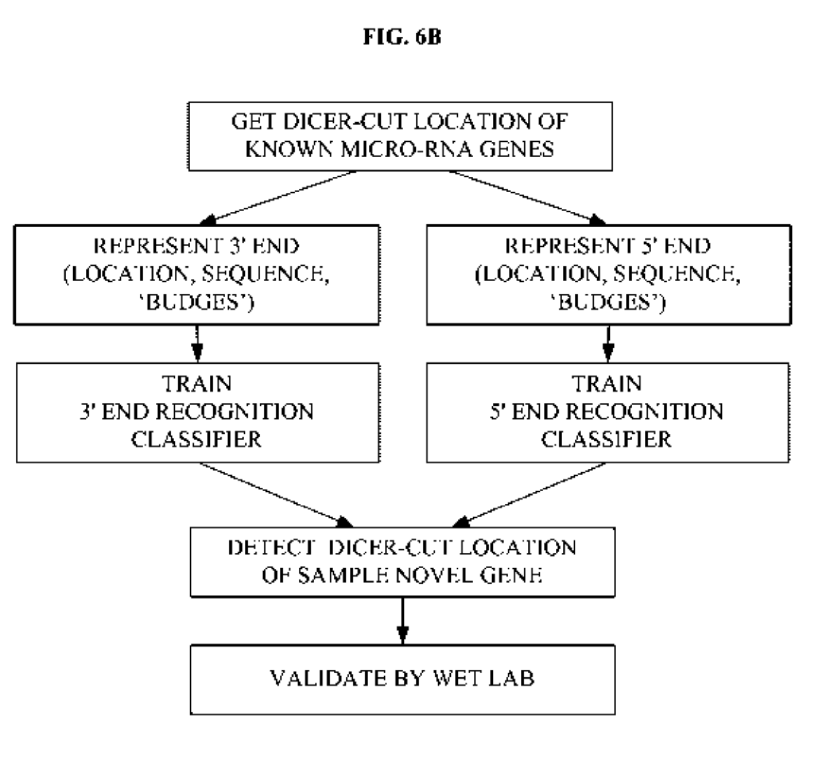

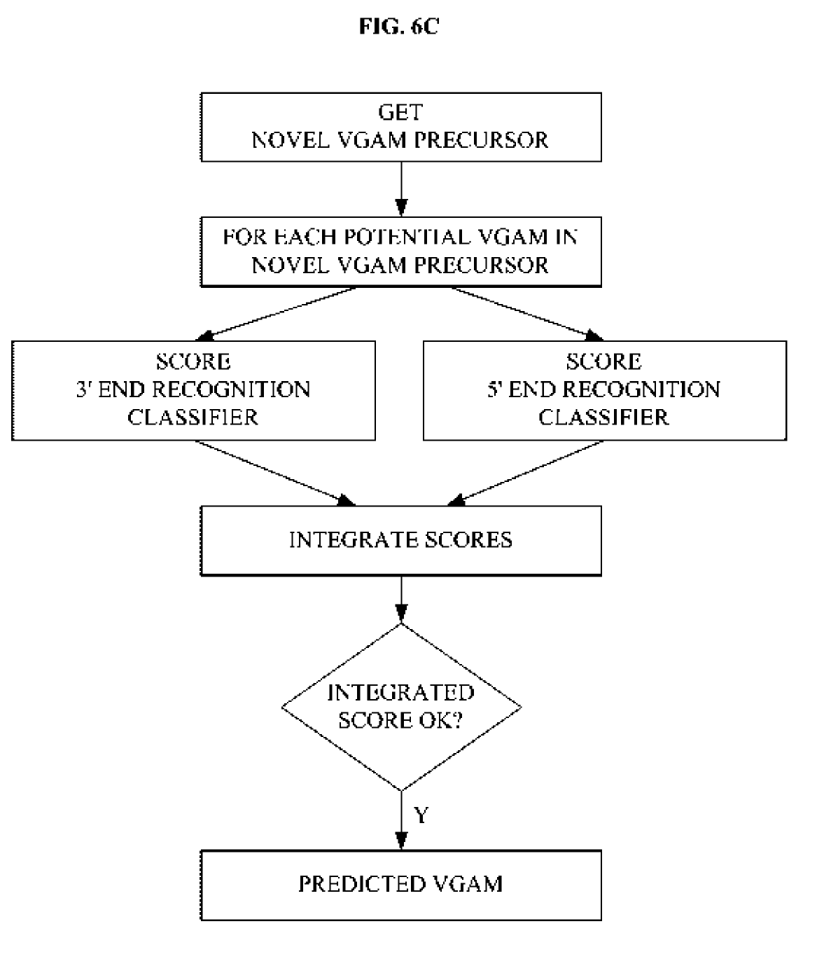

A dicer-cut location detector 116 operative to bioinformatically detect the location on a hairpin shaped sequence which is enzymatically cut by DICER COMPLEX of FIG. 1. The dicer-cut location detector 116 is further described hereinbelow with reference to FIG. 6A.

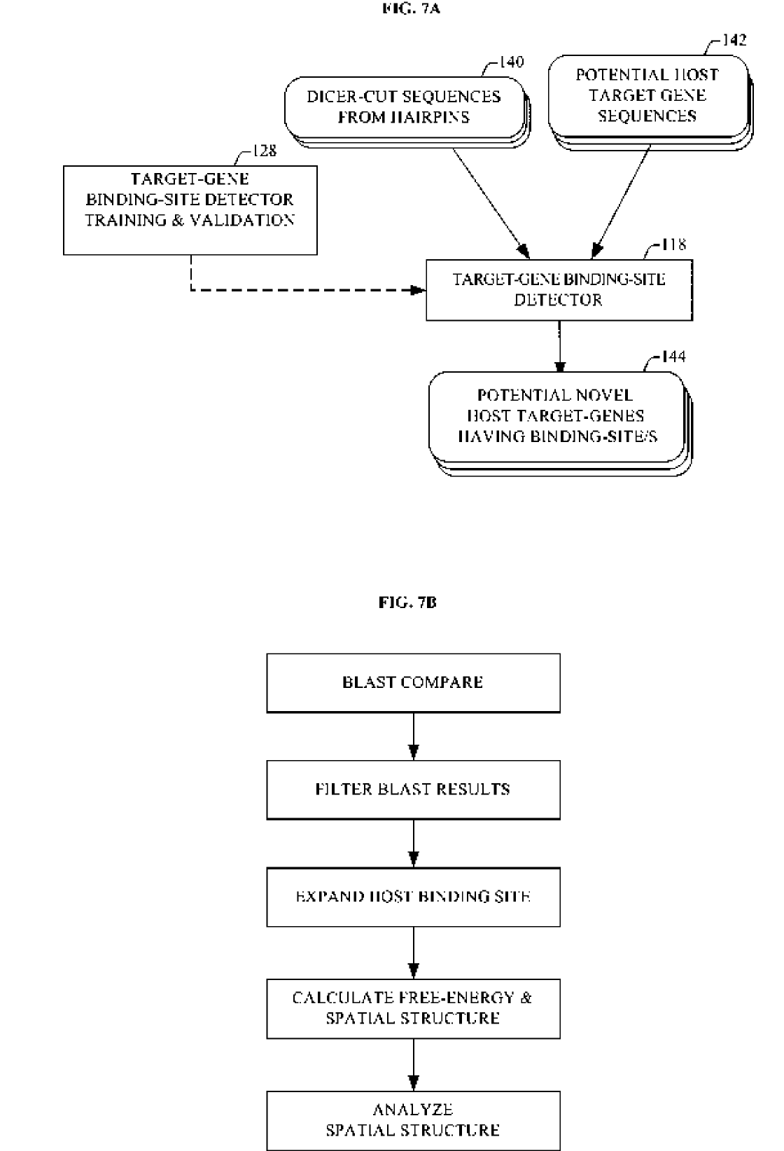

A target-gene binding-site detector 118 operative to bioinformatically detect host target having binding sites, the nucleotide sequence of which is partially complementary to that of a given genomic sequence, such as a sequence cut by DICER COMPLEX of FIG. 1. The target-gene binding-site detector 118 is further described hereinbelow with reference to FIGS. 7A and 7B.

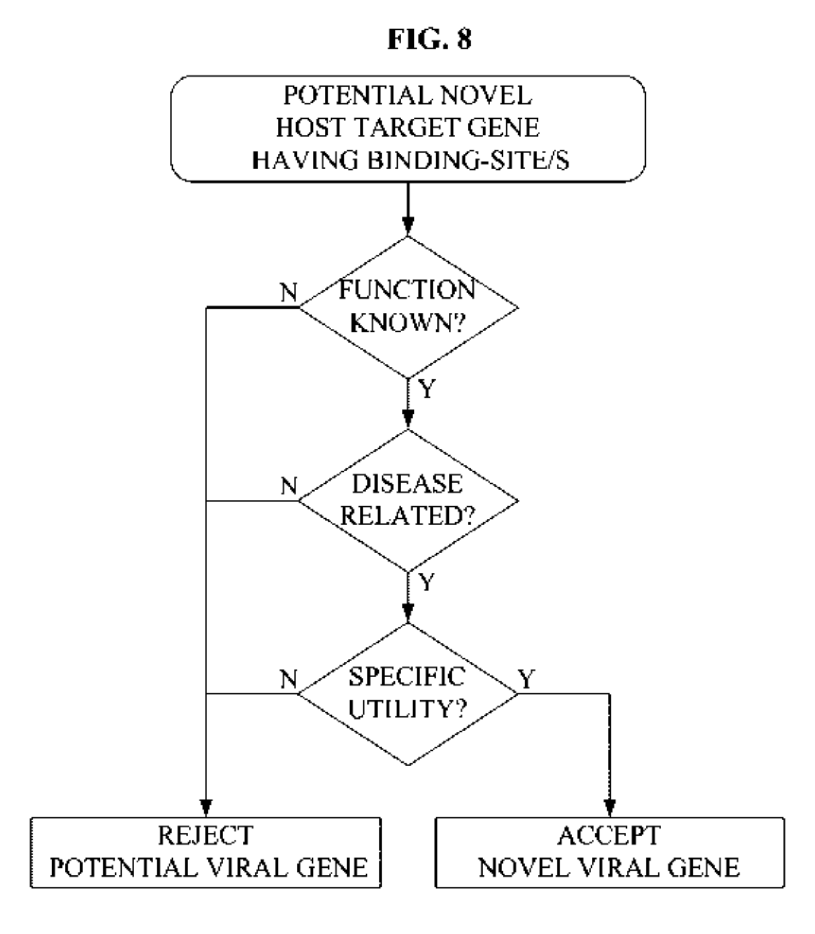

A function & utility analyzer 120 operative to analyze function and utility of host target, in order to identify host target which have a significant clinical function and utility. The function & utility analyzer 120 is further described hereinbelow with reference to FIG. 8.

Hardware implementation of the bioinformatic gene detection engine 100 is important, since significant computing power is preferably required in order to perform the computation of bioinformatic gene detection engine 100 in reasonable time and cost. As an example, it is estimated that using one powerful 8-processor PC Server, over 30 months of computing time (at 24 hours per day) would be required in order to detect all miRNA genes in human EST data, and their respective binding sites.

For example, in order to address this challenge at reasonable time and cost, a preferred embodiment of the present invention may comprise a cluster of a large number of personal computers (PCs), such as 100 PCs (Pentium IV, 1.7 GHz, with 40 GB storage each), connected by Ethernet to several strong servers, such as 4 servers (2-CPU, Xeon 2.2 GHz, with 200 GB storage each), combined with an 8-processor server (8-CPU, Xeon 550 Mhz w/8 GB RAM) connected via 2 HBA fiber-channels to an EMC Clariion 100-disks, 3.6 Terabyte storage device. Additionally, preferably an efficient database computer program, such as Microsoft™ SQL-Server database computer program is used and is optimized to the specific requirements of bioinformatic gene detection engine 100. Furthermore, the PCs are preferably optimized to operate close to 100% CPU usage continuously, as is known in the art. Using suitable hardware and software may preferably reduce the required calculation time in the abovementioned example from 30 months to 20 days.

It is appreciated that the abovementioned hardware configuration is not meant to be limiting, and is given as an illustration only. The present invention may be implemented in a wide variety of hardware and software configurations.

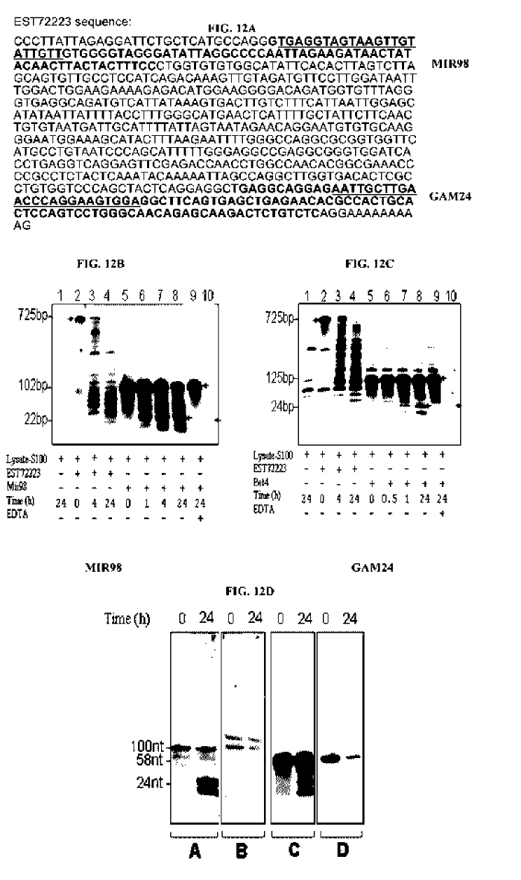

The present invention discloses 349 novel viral genes of the VGAM group of genes, which have been detected bioinformatically, as described hereinbelow with reference to FIG. 1 through FIG. 8. Laboratory confirmation of 4 genes of the GAM group of genes is described hereinbelow with reference to FIGS. 12 through 14.

Reference is now made to FIG. 3 which is a simplified flowchart illustrating operation of a mechanism for training of a computer system to recognize the novel genes of the present invention. This mechanism is a preferred implementation of the bioinformatic gene detection engine training & validation 110 described hereinabove with reference to FIG. 2.

Bioinformatic gene detection engine training & validation 110 of FIG. 2 begins by training the bioinformatic gene detection engine to recognize known miRNA genes, as designated by numeral 122. This training step comprises hairpin detector training & validation 124, further described hereinbelow with reference to FIG. 12 A, dicer-cut location detector training & validation 126, further described hereinbelow with reference to FIGS. 6A and 6B, and target-gene binding-site detector training & validation 128, further described hereinbelow with reference to FIG. 7A.

Next, the bioinformatic gene detection engine 100 is used to bioinformatically detect sample novel genes, as designated by numeral 130. An example of a sample novel gene thus detected is described hereinbelow with reference to FIG. 12.

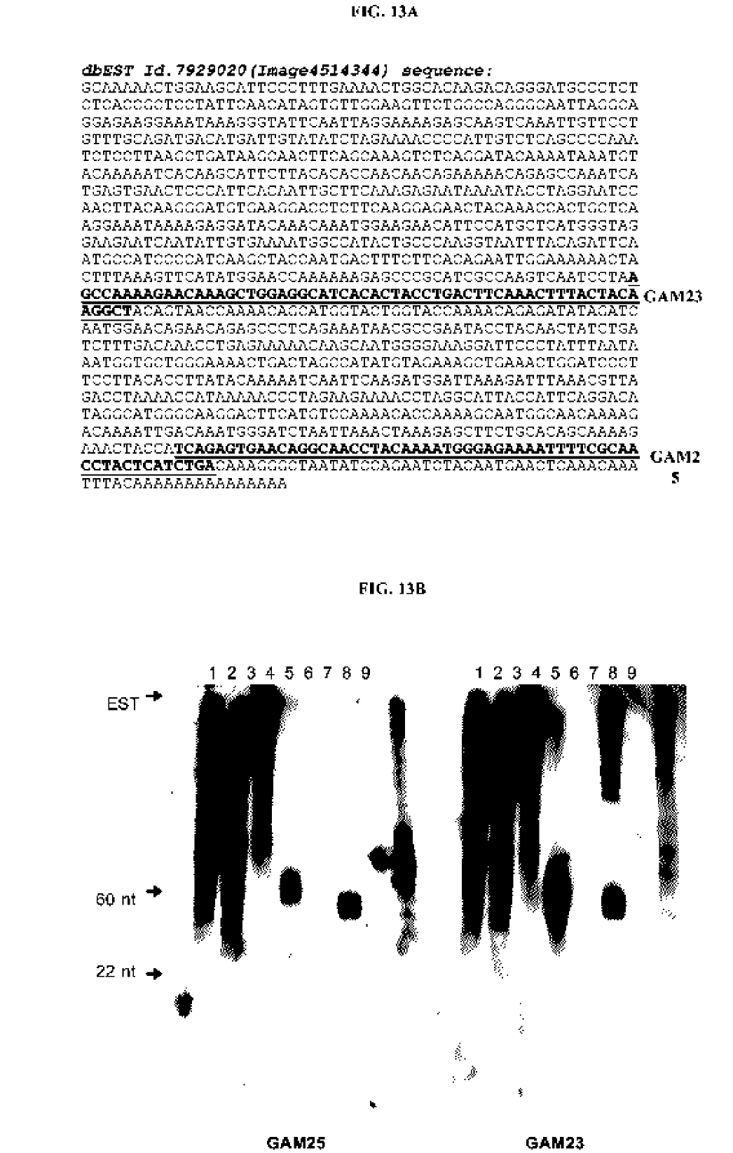

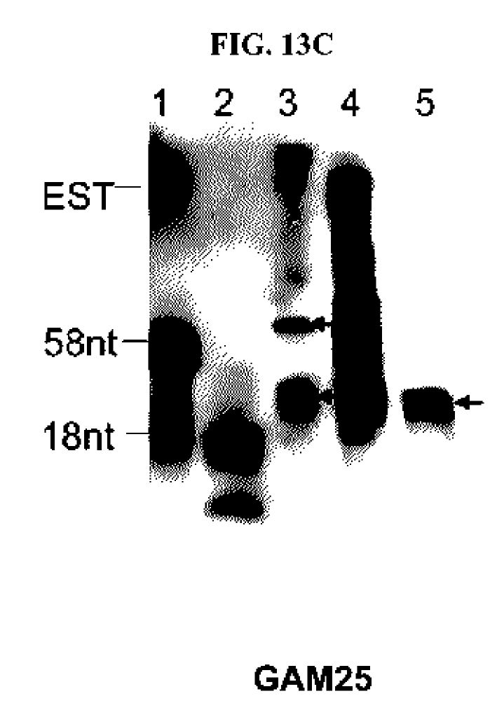

Finally, wet lab experiments are preferably conducted in order to validate expression and preferably function the sample novel genes detected by the bioinformatic gene detection engine 100 in the previous step. An example of wet-lab validation of the abovementioned sample novel gene bioinformatically detected by the system is described hereinbelow with reference to FIGS. 13A and 13B.

Reference is now made to FIG. 4A which is a simplified block diagram of a preferred implementation of the noncoding genomic sequence detector 112 described hereinabove with reference to FIG. 2. Non-protein coding genomic sequence detector 112 of FIG. 2 preferably receives as input at least two types of published genomic data: expressed RNA data 102, including EST data and mRNA data, and sequenced DNA data 104. After its initial training, indicated by numeral 134, and based on the abovementioned input data, the non-protein coding genomic sequence detector 112 produces as output a plurality of non-protein coding genomic sequences 136. Preferred operation of the non-protein coding genomic sequence detector 112 is described hereinbelow with reference to FIG. 4B.

Reference is now made to FIG. 4B which is a simplified flowchart illustrating a preferred operation of the noncoding genomic sequence detector 112 of FIG. 2. Detection of non-protein coding genomic sequences to be further analyzed by the system generally preferably progresses in one of the following two paths.

A first path for detecting non-protein coding genomic sequences begins by receiving a plurality of known RNA sequences, such as EST data. Each RNA sequence is first compared to all known protein-coding sequences, in order to select only those RNA sequences which are non-protein coding. This can preferably be performed by BLAST comparison of the RNA sequence to known protein coding sequences. The abovementioned BLAST comparison to the DNA preferably also provides the localization of the RNA on the DNA.

Optionally, an attempt may be made to “expend” the non-protein RNA sequences thus found, by searching for transcription start and end signals, upstream and downstream of location of the RNA on the DNA respectively, as is well known in the art.

A second path for detecting non-protein coding genomic sequences starts by receiving DNA sequences. The DNA sequences are parsed into non protein coding sequences, based on published DNA annotation data: extracting those DNA sequences which are between known protein coding sequences. Next, transcription start and end signals are sought. If such signals are found, and depending on their “strength”, probable expressed non-protein coding genomic sequences are yielded.

Reference is now made to FIG. 5A which is a simplified block diagram of a preferred implementation of the hairpin detector 114 described hereinabove with reference to FIG. 2.

The goal of the hairpin detector 114 is to detect “hairpin” shaped genomic sequences, similar to those of known miRNA genes. As mentioned hereinabove with reference to FIG. 1, a “hairpin” genomic sequence refers to a genomic sequence which “folds onto itself” forming a hairpin like shape, due to the fact that nucleotide sequence of the first half of the nucleotide sequence is an accurate or

The hairpin detector 114 of FIG. 2 receives as input a plurality of non-protein coding genomic sequences 136 of FIG. 4A, and after a phase of hairpin detector training & validation 124 of FIG. 3, is operative to detect and output “hairpin shaped” sequences found in the input expressed non-protein coding sequences, designated by numeral 138.

The phase of hairpin detector training & validation 124 is an iterative process of applying the hairpin detector 114 to known hairpin shaped miRNA genes, calibrating the hairpin detector 114 such that it identifies the training set of known hairpins, as well as sequences which are similar thereto. Preferred operation of the hairpin detector 114 is described hereinbelow with reference to FIG. 5B.

Reference is now made to FIG. 5B which is a simplified flowchart illustrating a preferred operation of the hairpin detector 114 of FIG. 2.

A hairpin structure is a two dimensional folding structure, resulting from the nucleotide sequence pattern: the nucleotide sequence of the first half of the hairpin sequence is an inversed-reversed sequence of the second half thereof. Different methodologies are known in the art for detection of various two dimensional and three dimensional hairpin structures.

In a preferred embodiment of the present invention, the hairpin detector 114 initially calculates possible 2-dimensional (2D) folding patterns of a given one of the non-protein coding genomic sequences 136, preferably using a 2D folding algorithm based on free-energy calculation, such as the Zucker algorithm, as is well known in the art.

Next, the hairpin detector 114 analyzes the results of the 2D folding, in order to determine the presence, and location of hairpin structures. A 2D folding algorithm typically provides as output a listing of the base-pairing of the 2D folded shape, i.e. a listing of which all two pairs of nucleotides in the sequence which will bond. The goal of this second step, is to asses this base-pairing listing, in order to determine if it describes a hairpin type bonding pattern.

The hairpin detector 114 then assess those hairpin structures found by the previous step, comparing them to hairpins of known miRNA genes, using various parameters such as length, free-energy, amount and type of mismatches, etc. Only hairpins that bear statistically significant resemblance of the population of hairpins of known mRNAs, according to the abovementioned parameters are accepted.

Lastly, the hairpin detector 114 attempts to select those hairpin structures which are as stable as the hairpins of know miRNA genes. This may be achieved in various manners. A preferred embodiment of the present invention utilizes the following methodology comprising three steps:

First, the hairpin detector 114 attempts to group potential hairpins into “families” of closely related hairpins. As is known in the art, a free-energy calculation algorithm, typically provides multiple “versions” each describing a different possible 2D folding pattern for the given genomic sequence, and the free energy of such possible folding. The hairpin detector 114 therefore preferably assesses all hairpins found on all “versions”, grouping hairpins which appear in different versions, but which share near identical locations into a common “family” of hairpins. For example, all hairpins in different versions, the center of which is within 7 nucleotides of each other may preferably be grouped to a single “family”.

Next, hairpin “families” are assessed, in order to select only those families which represent hairpins that are as stable as those of known mRNA hairpins. For example, preferably only families which are represented in at least 65% of the free-energy calculation 2D folding versions, are considered stable.

Finally, an attempt is made to select the most suitable hairpin from each selected family. For example, preferably the hairpin which appears in more versions than other hairpins, and in versions the free-energy of which is lower, may be selected.

Reference is now made to FIG. 6A which is a simplified block diagram of a preferred implementation of the dicer-cut location detector 116 described hereinabove with reference to FIG. 2.

The goal of the dicer-cut location detector 116 is to detect the location in which DICER COMPLEX of FIG. 1, comprising the enzyme Dicer, would “dice” the given hairpin sequence, similar to VGAM FOLDED PRECURSOR RNA, yielding VGAM RNA both of FIG. 1.

The dicer-cut location detector 116 of FIG. 2 therefore receives as input a plurality of hairpins on genomic sequences 138 of FIG. 5A, which were calculated by the previous step, and after a phase of dicer-cut location detector training & validation 126 of FIG. 3, is operative to detect a respective plurality of dicer-cut sequences from hairpins 140, one for each hairpin.

In a preferred embodiment of the present invention, the dicer-cut location detector 116 preferably uses a combination of neural networks, Bayesian networks, Markovian modeling, and Support Vector Machines (SVMs) trained on the known dicer-cut locations of known miRNA genes, in order to detect dicer-cut locations. Dicer-cut location detector training & validation 126, which is further described hereinbelow with reference to FIG. 6B.

Reference is now made to FIG. 6 B which is a simplified flowchart illustrating a preferred implementation of dicer-cut location detector training & validation 126 of FIG. 3. Dicer-cut location detector 116 first preprocesses known mRNA hairpins and their respective dicer-cut locations, so as to be able to properly analyze them and train the detection system accordingly:

The folding pattern is calculated for each known mRNA, preferably based on free-energy calculation, and the size of the hairpin, the size of the loop at the center of the hairpin, and “bulges” (i.e. mismatched base-pairs) in the folded hairpin are noted.

The dicer-cut location, which is known for known miRNA genes, is noted relative to the above, as well as to the nucleotides in each location along the hairpin. Frequency of identity of nucleotides, and nucleotide-pairing, relative to their location in the hairpin, and relative to the known dicer-cut location in the known miRNA genes is analyzed and modeled.

Different techniques are well known in the art for analysis of existing pattern from a given “training set” of species belonging to a genus, which techniques are then capable, to a certain degree, to detect similar patterns in other species not belonging to the training-set genus. Such techniques include, but are not limited to neural networks, Bayesian networks, Support Vector Machines (SVM), Genetic Algorithms, Markovian modeling, and others, as is well known in the art.

Using such techniques, preferably a combination of several of the above techniques, the known hairpins are represented as a several different networks (such as neural, Bayesian, or SVM) input and output layers. Both nucleotide, and “bulge” (i.e. nucleotide pairing or mismatch) are represented for each position in the hairpin, at the input layer, and a corresponding true/false flag at each position, indicating whether it was diced by dicer at the output layer. Multiple networks are preferably used concurrently, and the results therefrom are integrated and further optimized. Markovian modeling may also be used to validate the results and enhance their accuracy. Finally, the bioinformatic detection of dicer-cut location of a sample novel is confirmed by wet-lab experimentation.

Reference is now made to FIG. 7A which is a simplified block diagram of a preferred implementation of the target-gene binding-site detector 118 described hereinabove with reference to FIG. 2. The goal of the target-gene binding-site detector 118 is to detect a BINDING SITE of FIG. 1, located in an untranslated region of the RNA of a known gene, the nucleotide sequence of which BINDING SITE is at least partially complementary to that of a VGAM RNA of FIG. 1, thereby determining that the abovementioned known gene is a target gene of VGAM of FIG. 1.

The target-gene binding-site detector 118 of FIG. 2 therefore receives as input a plurality of dicer-cut sequences from hairpins 140 of FIG. 6A which were calculated by the previous step, and a plurality of potential target gene sequences 142 which derive sequence DNA data 104 of FIG. 2, and after a phase of target-gene binding-site detector training & validation 128 of FIG. 3, is operative to detect target-genes having binding site/s 144 the nucleotide sequence of which is at least partially complementary to that of each of the plurality of dicer-cut sequences from hairpins 140. Preferred operation of the target-gene binding-site detector is further described hereinbelow with reference to FIG. 7B.

Reference is now made to FIG. 7B which is a simplified flowchart illustrating a preferred operation of the target-gene binding-site detector 118 of FIG. 2. In a preferred embodiment of the present invention, the target-gene binding-site detector 118 first performs a BLAST comparison of the nucleotide sequence of each of the plurality of dicer-cut sequences from hairpins 140, to the potential target gene sequences 142, in order to find crude potential matches. Blast results are then filtered to results which are similar to those of known binding sites (e.g. binding sites of miRNA genes Lin-4 and Let-7 to target genes Lin-14, Lin-41, Lin 28 etc.). Next the binding site is expanded, checking if nucleotide sequenced immediately adjacent to the binding site found by BLAST, may improve the match. Suitable binding sites, then are computed for free-energy and spatial structure. The results are analyzed, selecting only those binding sites, which have free-energy and spatial structure similar to that of known binding sites.

Reference is now made to FIG. 8 which is a simplified flowchart illustrating a preferred operation of the function & utility analyzer 120 described hereinabove with reference to FIG. 2. The goal of the function & utility analyzer 120 is to determine if a potential target gene is in fact a valid clinically useful target gene. Since a potential novel VGAM gene binding a binding site in the UTR of a target gene is understood to inhibit expression of that target gene, and if that target gene is shown to have a valid clinical utility, then in such a case it follows that the potential novel gene itself also has a valid useful function which is the opposite of that of the target gene.

The function & utility analyzer 120 preferably receives as input a plurality of potential novel target genes having binding-site/s 144, generated by the target-gene binding-site detector 118, both of FIG. 7A. Each potential gene, is evaluated as follows:

First the system first checks to see if the function of the potential target gene is scientifically well established. Preferably, this can be achieved bioinformatically by searching various published data sources presenting information on known function of proteins. Many such data sources exist and are published as is well known in the art.

Next, for those target genes the function of which is scientifically known and is well documented, the system then checks if scientific research data exists which links them to known diseases. For example, a preferred embodiment of the present invention utilizes the OMIM™ database published by NCBI, which summarizes research publications relating to genes which have been shown to be associated with diseases.

Finally, the specific possible utility of the target gene is evaluated. While this process too may be facilitated by bioinformatic means, it might require human evaluation of published scientific research regarding the target gene, in order to determine the utility of the target gene to the diagnosis and or treatment of specific disease. Only potential novel genes, the target-genes of which have passed all three examinations, are accepted as novel genes.

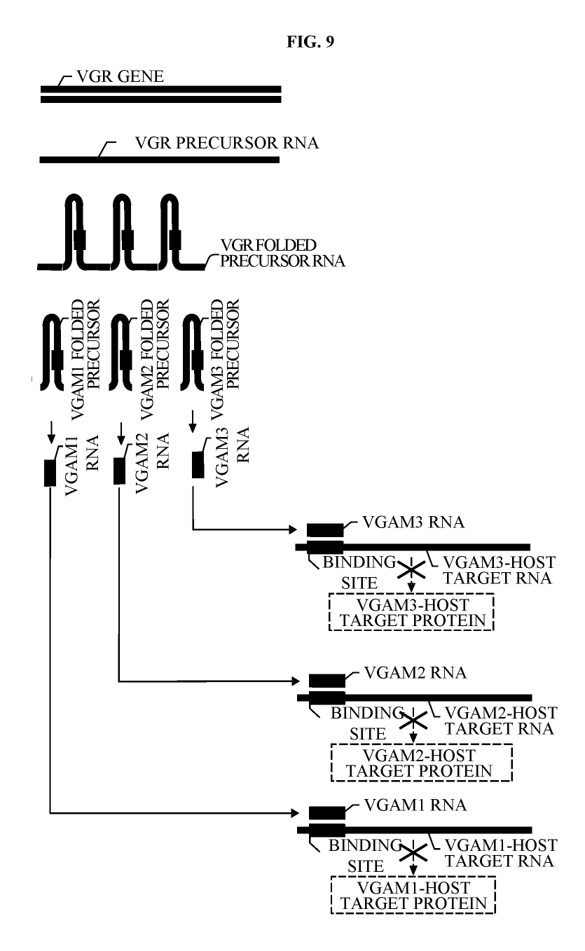

Reference is now made to FIG. 9, which is a simplified diagram describing a novel bioinformatically detected group of regulatory genes, referred to here as Viral Genomic Record (VGR) genes, that encode an “operon-like” cluster of novel viral miRNA-like genes, each modulating expression of a plurality of host target genes, the function and utility of which target genes is known.

VGR GENE (Viral Genomic Record Gene) is gene of a novel bioinformatically detected group of regulatory, non protein coding, RNA genes. The method by which VGR is detected is described hereinabove with reference to FIGS. 1-9.

VGR GENE encodes an RNA molecule, typically several hundred nucleotides long, designated VGR PRECURSOR RNA.

VGR PRECURSOR RNA folds spatially, as illustrated by VGR FOLDED PRECURSOR RNA, into a plurality of what is known in the art as “hairpin structures. The nucleotide sequence of VGR PRECURSOR RNA comprises a plurality of segments, the first half of each such segment having a nucleotide sequence which is at least a partial inversed-reversed sequence of the second half thereof, thereby causing formation of a plurality of “hairpin” structures, as is well known in the art.

VGR FOLDED PRECURSOR RNA is naturally processed by cellular enzymatic activity, into 3 separate hairpin shaped RNA segments, each corresponding to VGAM FOLDED PRECURSOR RNA of FIG. 1, designated VGAM1 FOLDED PRECURSOR, VGAM2 FOLDED PRECURSOR and VGAM3 FOLDED PRECURSOR respectively.

The above mentioned VGAM precursors, are diced by DICER COMPLEX of FIG. 1, yielding short RNA segments of about 22 nucleotides in length, each corresponding to VGAM RNA of FIG. 1, designated VGAM1 RNA, VGAM2 RNA and VGAM3 RNA respectively.

VGAM1 RNA, VGAM2 RNA and VGAM3 RNA each bind complementarily to binding sites located in untranslated regions of respective host target, designated VGAM1-HOST TARGET RNA, VGAM 2-HOST TARGET RNA and VGAM3-HOST TARGET RNA respectively. This binding inhibits translation of the respective target proteins designated VGAM1-HOST TARGET PROTEIN, VGAM2-HOST TARGET PROTEIN and VGAM3-HOST TARGET PROTEIN respectively.

The structure of VGAM genes comprised in a VGR GENE, and their mode of modulation of expression of their respective target genes is described hereinabove with reference to FIG. 1. The bioinformatic approach to detection of VGAM genes comprised in a VGR GENE is described hereinabove with reference to FIGS. 1 through 9.

The present invention discloses 426 novel viral genes of the VGR group of genes, which have been detected bioinformatically, as described hereinbelow with reference to FIG. 1 through FIG. 9. Laboratory confirmation of three genes of the VGR group of genes is described hereinbelow with reference to FIGS. 12A through 14B.

In summary, the current invention discloses a very large number of novel viral VGR genes, each of which encodes a plurality of VGAM genes, which in turn may modulate expression of a plurality of host target proteins.

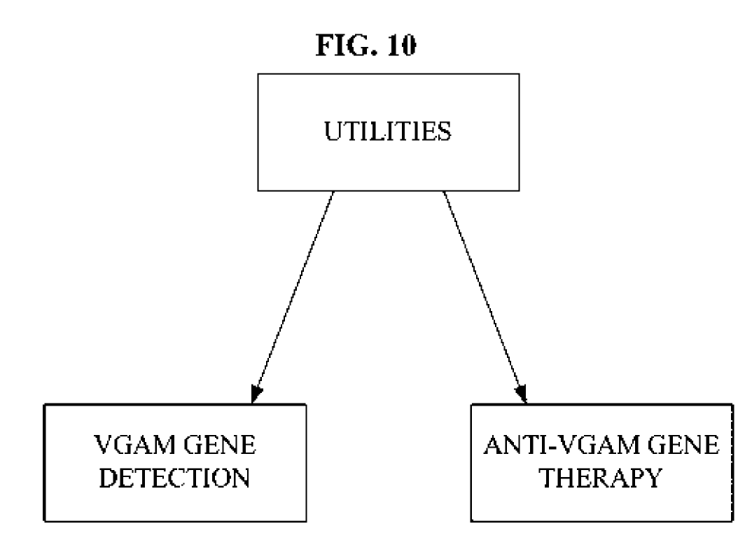

Reference is now made to FIG. 10 which is a block diagram illustrating different utilities of genes of the novel group of genes of the present invention referred to here as VGAM genes and VGR genes.

The present invention discloses a first plurality of novel genes referred to here as VGAM genes, and a second plurality of operon-like genes referred to here as VGR genes, each of the VGR genes encoding a plurality of VGAM genes. The present invention further discloses a very large number of known target-genes, which are bound by, and the expression of which is modulated by each of the novel genes of the present invention. Published scientific data referenced by the present invention provides specific, substantial, and credible evidence that the abovementioned target genes modulated by novel genes of the present invention, are associated with various diseases. Specific novel genes of the present invention, target genes thereof and diseases associated there with, are described hereinbelow with reference to FIG. 1 through FIG. 8. It is therefore appreciated that a function of VGAM genes and VGR genes of the present invention is modulation of expression of target genes related to known diseases, and that therefore utilities of novel genes of the present invention include diagnosis and treatment of the abovementioned diseases. FIG. 10 describes various types of diagnostic and therapeutic utilities of novel genes of the present invention.

A utility of novel genes of the present invention is detection of VGAM genes and of VGR genes. It is appreciated that since VGAM genes and VGR genes modulate expression of disease related target genes, that detection of expression of VGAM genes in clinical scenarios associated with said diseases is a specific, substantial and credible utility. Diagnosis of novel genes of the present invention may preferably be implemented by RNA expression detection techniques, including but not limited to biochips, as is well known in the art. Diagnosis of expression of genes of the present invention may be useful for research purposes, in order to further understand the connection between the novel genes of the present invention and the abovementioned related diseases, for disease diagnosis and prevention purposes, and for monitoring disease progress.

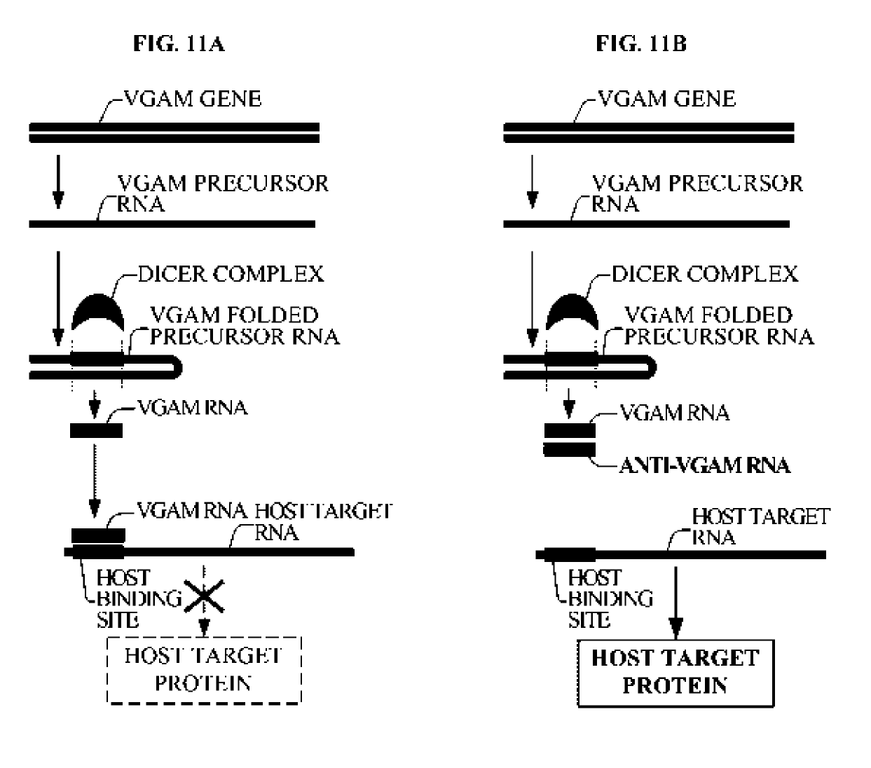

Another utility of novel genes of the present invention is anti-VGAM gene therapy, a mode of therapy which allows up regulation of a disease related target-gene of a novel VGAM gene of the present invention, by lowering levels of the novel VGAM gene which naturally inhibits expression of that target gene. This mode of therapy is particularly useful with respect to target genes which have been shown to be under-expressed in association with a specific disease. Anti-VGAM gene therapy is further discussed hereinbelow with reference to FIGS. 11A and 11B.

A further utility of novel genes of the present invention is VGAM replacement therapy, a mode of therapy which achieves down regulation of a disease related target-gene of a novel VGAM gene of the present invention, by raising levels of the VGAM gene which naturally inhibits expression of that target gene. This mode of therapy is particularly useful with respect to target genes which have been shown to be over-expressed in association with a specific disease. VGAM replacement therapy involves introduction of supplementary VGAM gene products into a cell, or stimulation of a cell to produce excess VGAM gene products. VGAM replacement therapy may preferably be achieved by transfecting cells with an artificial DNA molecule encoding a VGAM gene, which causes the cells to produce the VGAM gene product, as is well known in the art.

Yet a further utility of novel genes of the present invention is modified VGAM therapy. Disease conditions are likely to exist, in which a mutation in a binding site of a VGAM gene prevents natural VGAM gene to effectively bind inhibit a disease related target-gene, causing up regulation of that target gene, and thereby contributing to the disease pathology. In such conditions, a modified VGAM gene is designed which effectively binds the mutated VGAM binding site, i.e. is an effective anti-sense of the mutated VGAM binding site, and is introduced in disease effected cells. Modified VGAM therapy is preferably achieved by transfecting cells with an artificial DNA molecule encoding the modified VGAM gene, which causes the cells to produce the modified VGAM gene product, as is well known in the art.

An additional utility of novel genes of the present invention is induced cellular differentiation therapy. As aspect of the present invention is finding genes which determine cellular differentiation, as described hereinabove with reference to FIG. 11. Induced cellular differentiation therapy comprises transfection of cell with such VGAM genes thereby determining their differentiation as desired. It is appreciated that this approach may be widely applicable, inter alia as a means for auto transplantation harvesting cells of one cell-type from a patient, modifying their differentiation as desired, and then transplanting them back into the patient. It is further appreciated that this approach may also be utilized to modify cell differentiation in vivo, by transfecting cells in a genetically diseased tissue with a cell-differentiation determining VGAM gene, thus stimulating these cells to differentiate appropriately.

Reference is now made to FIGS. 11A and 11B, simplified diagrams which when taken together illustrate anti-VGAM gene therapy mentioned hereinabove with reference to FIG. 10. A utility of novel genes of the present invention is anti-VGAM gene therapy, a mode of therapy which allows up regulation of a disease related target-gene of a novel VGAM gene of the present invention, by lowering levels of the novel VGAM gene which naturally inhibits expression of that target gene. FIG. 11A shows a normal VGAM gene, inhibiting translation of a target gene of VGAM gene, by binding to a BINDING SITE found in an untranslated region of TARGET RNA, as described hereinabove with reference to FIG. 1.

FIG. 11B shows an example of anti-VGAM gene therapy. ANTI-VGAM RNA is short artificial RNA molecule the sequence of which is an anti-sense of VGAM RNA. Anti-VGAM treatment comprises transfecting diseased cells with ANTI-VGAM RNA, or with a DNA encoding thereof. The ANTI-VGAM RNA binds the natural VGAM RNA, thereby preventing binding of natural VGAM RNA to its BINDING SITE. This prevents natural translation inhibition of TARGET RNA by VGAM RNA, thereby up regulating expression of TARGET PROTEIN.

It is appreciated that anti-VGAM gene therapy is particularly useful with respect to target genes which have been shown to be under-expressed in association with a specific disease.

Reference is now made to FIG. 12A which is an annotated sequence of an EST comprising a novel gene detected by the gene detection system of the present invention. FIG. 12A shows the nucleotide sequence of a known human non-protein coding EST (Expressed Sequence Tag), identified as EST72223. It is appreciated that the sequence of this EST comprises sequences of one known miRNA gene, identified as MIR98, and of one novel GAM gene, referred to here as GAM24, detected by the bioinformatic gene detection system of the present invention, described hereinabove with reference to FIG. 2.

Reference is now made to FIGS. 12B and 12C that are pictures of laboratory results, which when taken together demonstrate laboratory confirmation of expression of the bioinformatically detected novel gene of FIG. 12A. Reference is now made to FIG. 12B which is a Northern blot analysis of MIR-98 and EST72223 transcripts. MIR-98 and EST72223 were reacted with MIR-98 and GAM24 probes as indicated in the figure. It is appreciated that the probes of both MIR-98 and GAM24 reacted with EST72223, indicating that EST72223 contains the sequences of MIR-98 and of GAM24. It is further appreciated that the probe of GAM24 does not cross-react with MIR-98.

Reference is now made to FIG. 12C. A Northern blot analysis of EST72223 and MIR-98 transfections were performed, subsequently marking RNA by the MIR-98 and GAM24 probes. Left, Northern reacted with MIR-98, Right, Northern reacted with GAM24. The molecular Sizes of EST72223, MIR-98 and GAM24 are indicated by arrows. Hela are control cells that have not been introduced to exogenous RNA. EST and MIR-98 Transfections are RNA obtained from Hela transfected with EST72223 and MIR-98, respectively. MIR-98 and EST are the transcripts used for the transfection experiment. The results indicate that EST72223, when transfected into Hela cells, is cut yielding known miRNA gene MIR-98 and novel miRNA gene GAM24.

Reference is now made to FIG. 12D, which is a Northern blot of a lisate experiment with MIR-98 and GAM24. Northern blot analysis of hairpins in EST72223. Left, Northern reacted with predicted Mir-98 hairpin probe, Right, Northern reacted with predicted GAM24 hairpin probe. The molecular size of EST Is indicated by arrow. The molecular sizes of Mir-98 and GAM24 are 80 nt and 100 nt, respectively as indicated by arrows. The 22 nt molecular marker is indicated by arrow. 1-Hela lysate; 2-EST incubated 4 h with Hela lysate; 3-EST without lysate; 4-Mir transcript incubated 4 h with Hela lysate; 5-Mir transcript incubated overnight with Hela lysate; 6-Mir transcript without lysate; 7-RNA extracted from Hela cells following transfection with Mir transcript.

Technical methods used in experiments, the results of which are depicted in FIGS. 12B, 12C and 12D are as follows:

Transcript preparations: Digoxigenin (DIG) labeled transcripts were prepared from EST72223 (TIGER), MIR98 and predicted precursor hairpins by using a DIG RNA labeling kit (Roche Molecular Biochemicals) according to the manufacture's protocol. Briefly, PCR products with T7 promoter at the 5″ end or T3 promoter at the 3″end were prepared from each DNA in order to use it as a template to prepare sense and antisense transcripts, respectively. MIR-98 was amplified using EST72223 as a template with T7miR98 forward primer: 5-″TAATACGACTCACTATAGGGTGAGGTAGTAAGTTGTATTGTT-3″ and T3miR98 reverse primer: 5″-AATTAACCCTCACTAAAGGGAAAGTAGTAAGTTGTATAGTT-3″ EST72223 was amplified with T7-EST 72223 forward primer: 5″-TAATACGACTCACTATAGGCCCTTATTAGAGGATTCTGCT-3″ and T3-EST72223 reverse primer: 5″-AATTAACCCTCACTAAAGGTTTTTTTTTCCTGAGACAGAGT-3″ Bet-4 was amplified using EST72223 as a template with Bet-4 forward primer: 5″-GAGGCAGGAGAATTGCTTGA-3″ and T3-EST72223 reverse primer: 5″-AATTAACCCTCACTAAAGGCCTGAGACAGAGTCTTGCTC-3″ The PCR products were cleaned and used for DIG-labeled or unlabeled transcription reactions with the appropriate polymerase. For transfection experiments, CAP reaction was performed by using a mMassage mMachine kit (Ambion).

Transfection procedure: Transfection of Hela cells was performed by using TransMessenger reagent (Qiagen) according to the manufacture's protocol. Briefly, Hela cells were seeded to 1-2×10\textcircled{}6 cells per plate a day before transfection. Two μg RNA transcripts were mixed with 8 μl Enhancer in a final volume of 100 μl, mixed and incubated at room temperature for 5 min. 16 μl TransMessenger reagent was added to the RNA-Enhancer, mixed and incubated for additional 10 min. Cell plates were washed with sterile PBS twice and then incubated with the transfection mix diluted with 2.5 ml DMEM medium without serum. Cells were incubated with transfection mix for three hours under their normal growth condition (370 C and 5% CO2) before the transfection mix was removed and a fresh DMEM medium containing serum was added to the cells. Cells were left to grow 48 hours before harvesting.

Target RNA cleavage assay: Cap-labeled target RNAs were generated using mMessage mMachine™ (Ambion). Caped RNA transcripts were preincubated at 30° C. for 15 min in supplemented Hela S100 obtained from Computer Cell Culture, Mos, Belgium. After addition of all components, final concentrations were 100 mM target RNA, 1 mM ATP, 0.2 mM GTP, 10 U/ml RNasin, 30 μg/ml creatine kinase, 25 mM creatine phosphate, and 50% S100 extract. Incubation was continued for 4 hours to overnight. Cleavage reaction was stopped by the addition of 8 volumes of proteinase K buffer (200 Mm Tris-Hcl, pH 7.5, 25 mM EDTA, 300 mM NaCl, and 2% SDS). Proteinase K, dissolved in 50 mM Tris-HCl, pH 8, 5 mM CaCl2, and 50% glycerol, was added to a final concentration of 0.6 mg/ml. Sample were subjected to phenol/chlorophorm extraction and kept frozen until analyzed by urea-TBE PAGE.

Northern analysis: RNAs were extracted from cells by using Tri-reagent according to the manufacture's protocol. The RNAs were dissolved in water and heated to 650 C to disrupt any association of the 25 nt RNA with larger RNA molecules. RNA were placed on ice and incubated for 30 min with PEG (MW=8000) in a final concentration of 5% and NaCl in a final concentration of 0.5M to precipitate high molecular weight nucleic acid. The RNAs were centrifuged at 10,000×g for 10 min to pellet the high molecular weight nucleic acid. The supernatant containing the low molecular weight RNAs was collected and three volumes of ethanol was added. The RNAs were placed at −200 C for at least two hours and then centrifuged at 10,000×g for 10 min. The pellets were dissolved in Urea-TBE buffer (1×tbe, 7M urea) for further analysis by a Northern blot.

RNA samples were boiled for 5 min before loading on 15%-8% polyacrylamide (19:1) gels containing 7M urea and 1×TBE. Gels were run in 1×TBE at a constant voltage of 300V and then transferred into a nylon membrane. The membrane was exposed to 3 min ultraviolet light to cross link the RNAs to the membrane. Hybridization was performed overnight with DIG-labeled probes at 420 C. Membranes were washed twice with SSC×2 and 0.2% SDS for 10 min. at 420 C and then washed twice with SSC×0.5 for 5 min at room temperature. The membrane was then developed by using a DIG luminescent detection kit (Roche) using anti DIG and CSPD reaction, according to the manufacture's protocol.

It is appreciated that the data presented in FIGS. 12A, 12B, 12C and 12D, when taken together validate the function of the bioinformatic gene detection engine 100 of FIG. 2. FIG. 12A shows a novel GAM gene bioinformatically detected by the bioinformatic gene detection engine 100, and FIGS. 12B, 12C and 12D show laboratory confirmation of the expression of this novel gene. This is in accord with the engine training and validation methodology described hereinabove with reference to FIG. 3.

Reference is now made to FIG. 13A which is an annotated sequence of an EST comprising a novel gene detected by the gene detection system of the present invention. FIG. 13A shows the nucleotide sequence of a known human non-protein coding EST (Expressed Sequence Tag), identified as EST 7929020. It is appreciated that the sequence of this EST comprises sequences of two novel GAM genes, referred to here as GAM23 and GAM25, detected by the bioinformatic gene detection system of the present invention, described hereinabove with reference to FIG. 2.

Reference is now made to FIG. 13B which presents pictures of laboratory results, that demonstrate laboratory confirmation of expression of the bioinformatically detected novel gene of FIG. 13A. Northern blot analysis of hairpins in EST7929020. Left, Northern reacted with predicted GAM25 hairpin probe, Right, Northern reacted with predicted GAM23 hairpin probe. The molecular size of EST is indicated by arrow. The molecular sizes of GAM23 and GAM25 are 60 nt, as indicated by arrow. The 22 nt molecular marker is indicated by arrow. 1-Hela lysate; 2-EST incubated 4 h with Hela lysate; 3-EST incubated overnight with Hela lysate; 4-EST without lysate; 5-GAM transcript; 6-GAM 22 nt marker; 7-GAM PCR probe; 8-RNA from control Hela cells; 9-RNA extracted from Hela cells following transfection with EST.

Reference is now made to FIG. 13C which is a picture of a Northern blot confirming Endogenous expression of bioinformatically detected gene GAM25 of FIG. 13A from in Hela cells. Northern was reacted with a predicted GAM25 hairpin probe. The molecular size of EST7929020 is indicated. The molecular sizes of GAM25 is 58 nt, as indicated. A 19 nt DNA oligo molecular marker is indicated. Endogenous expression of GAM25 in Hela total RNA fraction and in S-100 fraction is indicated by arrows. 1-GAM25 transcript; 2-GAM25 DNA oligo marker; 3-RNA from control Hela cells; 4-RNA extracted from Hela cells following transfection with EST; 5-RNA extracted from S-100 Hela lysate.

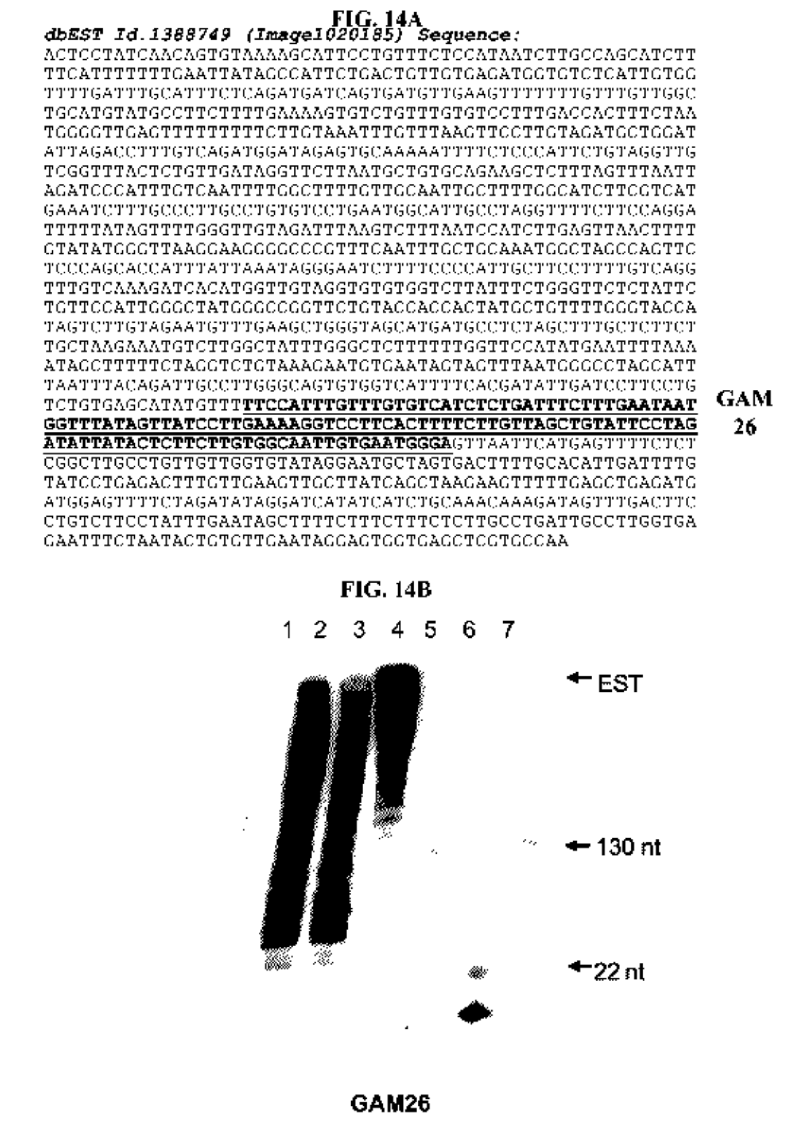

Reference is now made to FIG. 14A which is an annotated sequence of an EST comprising a novel gene detected by the gene detection system of the present invention. FIG. 14A shows the nucleotide sequence of a known human non-protein coding EST (Expressed Sequence Tag), identified as EST 1388749. It is appreciated that the sequence of this EST comprises sequence of a novel GAM gene, referred to here as GAM26, detected by the bioinformatic gene detection system of the present invention, described hereinabove with reference to FIG. 2.

Reference is now made to FIG. 14B which is a picture of Northern blot analysis, confirming expression of novel bioinformatically detected gene GAM26, and natural processing thereof from EST1388749. Northern reacted with predicted GAM26 hairpin probe. The molecular size of EST is indicated by arrow. The molecular sizes of GAM26 is 130 nt, as indicated by arrow. The 22 nt molecular marker is indicated by arrow. 1-Hela lysate; 2-EST incubated 4 h with Hela lysate; 3-EST incubated overnight with Hela lysate; 4-EST without lysate; 5-GAM transcript; 6-GAM 22 nt marker; 7-GAM PCR probe.

FIG. 1 further provides a conceptual description of a novel bioinformatically detected viral gene of the present invention, referred to here as Viral Genomic Address Messenger 15 (VGAM15) viral gene, which modulates expression of respective host target genes thereof, the function and utility of which host target genes is known in the art.

VGAM15 is a novel bioinformatically detected regulatory, non protein coding, viral micro RNA (miRNA) gene. The method by which VGAM15 was detected is described hereinabove with reference to FIGS. 1-8.

VGAM15 gene, herein designated VGAM GENE, is a viral gene contained in the genome of Vaccinia Virus. VGAM15 host target gene, herein designated VGAM HOST TARGET GENE, is a human gene contained in the human genome.

VGAM15 gene encodes a VGAM15 precursor RNA, herein designated VGAM PRECURSOR RNA. Similar to other miRNA genes, and unlike most ordinary genes, VGAM15 precursor RNA does not encode a protein. A nucleotide sequence identical or highly similar to the nucleotide sequence of VGAM15 precursor RNA is designated SEQ ID:1, and is provided hereinbelow with reference to the sequence listing part. Nucleotide sequence SEQ ID:1 is located at position 190678 relative to the genome of Vaccinia Virus.

VGAM15 precursor RNA folds onto itself, forming VGAM15 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM15 folded precursor RNA into VGAM15 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM15 RNA is designated SEQ ID:350, and is provided hereinbelow with reference to the sequence listing part.

VGAM15 host target gene, herein designated VGAM HOST TARGET GENE, encodes a corresponding messenger RNA, VGAM15 host target RNA, herein designated VGAM HOST TARGET RNA. VGAM15 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM15 RNA, herein designated VGAM RNA, binds complementarily to one or more host target binding sites located in untranslated regions of VGAM15 host target RNA, herein designated VGAM HOST TARGET RNA. This complementary binding is due to the fact that the nucleotide sequence of VGAM15 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM15 RNA, herein designated VGAM RNA, may have a different number of host target binding sites in untranslated regions of a VGAM15 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM15 RNA, herein designated VGAM RNA, to host target binding sites on VGAM15 host target RNA, herein designated VGAM HOST TARGET RNA, such as BINDING SITE I, BINDING SITE II and BINDING SITE III, inhibits translation of VGAM15 host target RNA into VGAM15 host target protein, herein designated VGAM HOST TARGET PROTEIN. VGAM host target protein is therefore outlined by a broken line.

It is appreciated that VGAM15 host target gene, herein designated VGAM HOST TARGET GENE, in fact represents a plurality of VGAM15 host target genes. The mRNA of each one of this plurality of VGAM15 host target genes comprises one or more host target binding sites, each having a nucleotide sequence which is at least partly complementary to VGAM15 RNA, herein designated VGAM RNA, and which when bound by VGAM15 RNA causes inhibition of translation of respective one or more VGAM15 host target proteins.

It is further appreciated by one skilled in the art that the mode of translational inhibition illustrated by FIG. 1 with specific reference to translational inhibition exerted by VGAM15 gene, herein designated VGAM GENE, on one or more VGAM15 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM15 is inhibition of expression of host target genes, as part of a novel viral mechanism of attacking a host. Accordingly, utilities of VGAM15 include diagnosis, prevention and treatment of viral infection by Vaccinia Virus. Specific functions, and accordingly utilities, of VGAM15 correlate with, and may be deduced from, the identity of the host target genes which VGAM15 binds and inhibits, and the function of these host target genes, as elaborated hereinbelow.

Nucleotide sequences of the VGAM15 precursor RNA, herein designated VGAM PRECURSOR RNA, and of the ‘diced’ VGAM15 RNA, herein designated VGAM RNA, and a schematic representation of the secondary folding of VGAM15 folded precursor RNA, herein designated VGAM FOLDED PRECURSOR RNA, of VGAM15 are further described hereinbelow with reference to Table 1.

Nucleotide sequences of host target binding sites, such as BINDING SITE-I, BINDING SITE-II and BINDING SITE-III of FIG. 1, found on VGAM15 host target RNA, and schematic representation of the complementarity of each of these host target binding sites to VGAM15 RNA, herein designated VGAM RNA, are described hereinbelow with reference to Table 2.

As mentioned hereinabove with reference to FIG. 1, a function of VGAM15 gene, herein designated VGAM is inhibition of expression of VGAM15 target genes. It is appreciated that specific functions, and accordingly utilities, of VGAM15 correlate with, and may be deduced from, the identity of the target genes which VGAM15 binds and inhibits, and the function of these target genes, as elaborated hereinbelow.

Calpain 2, (m/II) Large Subunit (CAPN2, Accession NM_{—}001748) is a VGAM15 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM15 RNA, herein designated VGAM RNA, also designated SEQ ID:350.

A function of VGAM15 is therefore inhibition of Calpain 2, (m/II) Large Subunit (CAPN2, Accession NM_{—}001748). Accordingly, utilities of VGAM15 include diagnosis, prevention and treatment of diseases and clinical conditions associated with CAPN2. Dishevelled Associated Activator of Morphogenesis 2 (DAAM2, Accession XM_{—}166434) is another VGAM15 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM15 RNA, herein designated VGAM RNA, also designated SEQ ID:350.

Another function of VGAM15 is therefore inhibition of Dishevelled Associated Activator of Morphogenesis 2 (DAAM2, Accession XM_{—}166434), a gene which controls cell polarity and movement during development. Accordingly, utilities of VGAM15 include diagnosis, prevention and treatment of diseases and clinical conditions associated with DAAM2. The function of DAAM2 has been established by previous studies. By sequencing randomly selected cDNAs corresponding to relatively long transcripts from brain, Nagase et al. (1997) identified a cDNA which they designated KIAA0381. The KIAA0381 cDNA encodes an 864-amino acid protein predicted to be involved in cell division. RT-PCR analysis detected expression of KIAA0381 in most tissues tested. Wnt (see OMIM Ref. No. 164975) signaling via the frizzled receptor (Fz; OMIM Ref. No. 600667) controls cell polarity and movement during development. Habas et al. (2001) reported that in human cells and during Xenopus embryogenesis, Wnt/Fz signaling activates the small GTPase Rho (OMIM Ref. No. 165390), a key regulator of cytoskeleton architecture. Wnt/Fz activation of Rho requires the cytoplasmic protein dishevelled (DVL; OMIM Ref. No. 601365) and a novel formin (see OMIM Ref. No. 136535) homology (FH) protein that they identified and named DAAM1 (OMIM Ref. No. 606626). Habas et al. (2001) identified DAAM2, which is identical to KIAA0381, as a protein that is closely related to DAAM1.

Full details of the abovementioned studies are described in the following publications, the disclosure of which are hereby incorporated by reference:
Habas, R.; Kato, Y.; He, X.: Wnt/Frizzled activation of Rho regulates vertebrate gastrulation and requires a novel Formin homology protein Daam1. Cell 107: 843-854, 2001.; and
Nagase, T.; Ishikawa, K.; Nakajima, D.; Ohira, M.; Seki, N.; Miyajima, N.; Tanaka, A.; Kotani, H.; Nomura, N.; Ohara, O.: Prediction of the coding sequences of unidentified human gene.

Further studies establishing the function and utilities of DAAM2 are found in John Hopkins OMIM database record ID 606627, and in sited publications numbered 1012 listed in the bibliography section hereinbelow, which are also hereby incorporated by reference. Selectin E (endothelial adhesion molecule 1) (SELE, Accession NM_{—}000450) is another VGAM15 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM15 RNA, herein designated VGAM RNA, also designated SEQ ID:350.

Another function of VGAM15 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM15 include diagnosis, prevention and treatment of diseases and clinical conditions associated with SELE. The function of SELE has been established by previous studies. Zheng et al. (2001) examined whether a polymorphism in the SELE gene, due to a G-to-T mutation (98G-T) in the untranslated region of exon 2, was related to premature coronary artery disease (CAD). Both lipid and nonlipid risk factors, including the ser128-to-arg substitution studied by Wenzel et al. (1994), were also assessed. The frequency of the 98G-T mutation was found to be significantly increased among male patients under 45 years of age and female patients under 55 years of age. After controlling for other CAD risk factors by multiple logistic analysis, the 98G-T mutation was still a significant predictor of premature CAD. The glaucomas are a group of optic neuropathies comprising the leading cause of irreversible blindness worldwide. Elevated intraocular pressure due to a reduction in normal aqueous outflow is a major causal risk factor. Wang et al. (2001) found that ELAM1, the earliest marker for the atherosclerotic plaque in the vasculature, was consistently present on trabecular meshwork cells in the outflow pathways of eyes with glaucomas of diverse etiology. They determined expression of ELAM1 to be controlled by activation of an interleukin-1 (see OMIM Ref. No. 147760) autocrine feedback loop through transcription factor NK-kappa-B (see OMIM Ref. No. 164011), and activity of this signaling pathway was shown to protect trabecular meshwork cells against oxidative stress. Wang et al. (2001) concluded that their findings characterized a protective stress response specific to the eye's aqueous outflow pathways and provided the first known diagnostic indicator of glaucomatous trabecular meshwork cells. They further indicated that common mechanisms contribute to the pathophysiology of the glaucomas and vascular diseases.

Full details of the abovementioned studies are described in the following publications, the disclosure of which are hereby incorporated by reference:
Wang, N.; Chintala, S. K.; Fini, M. E.; Schuman, J. S.: Activation of a tissue-specific stress response in the aqueous outflow pathway of the eye defines the glaucoma disease phenotype. Nature Med. 7: 304-309, 2001.; and
Zheng, F.; Chevalier, J. A.; Zhang, L. Q.; Virgil, D.; Ye, S. Q.; Kwiterovich, P. O.: An Hphl polymorphism in the Eselectin gene is associated with premature coronary artery disease.

Further studies establishing the function and utilities of SELE are found in John Hopkins OMIM database record ID 131210, and in sited publications numbered 2722-2729 listed in the bibliography section hereinbelow, which are also hereby incorporated by reference. KIAA0475 (Accession NM_{—}014864) is another VGAM15 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM15 RNA, herein designated VGAM RNA, also designated SEQ ID:350.

Another function of VGAM15 is therefore inhibition of KIAA0475 (Accession NM_{—}014864). Accordingly, utilities of VGAM15 include diagnosis, prevention and treatment of diseases and clinical conditions associated with KIAA0475. LOC222671 (Accession XM_{—}167094) is another VGAM15 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM15 RNA, herein designated VGAM RNA, also designated SEQ ID:350.

Another function of VGAM15 is therefore inhibition of LOC222671 (Accession XM_{—}167094). Accordingly, utilities of VGAM15 include diagnosis, prevention and treatment of diseases and clinical conditions associated with LOC222671. LOC254901 (Accession XM_{—}171878) is another VGAM15 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM15 RNA, herein designated VGAM RNA, also designated SEQ ID:350.

Another function of VGAM15 is therefore inhibition of LOC254901 (Accession XM_{—}171878). Accordingly, utilities of VGAM15 include diagnosis, prevention and treatment of diseases and clinical conditions associated with LOC254901. LOC257615 (Accession XM_{—}175293) is another VGAM15 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM15 RNA, herein designated VGAM RNA, also designated SEQ ID:350.

Another function of VGAM15 is therefore inhibition of LOC257615 (Accession XM_{—}175293). Accordingly, utilities of VGAM15 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 16 (VGAM16) viral gene, which modulates expression of respective host target genes thereof, the function and utility of which host target genes is known in the art.

VGAM16 is a novel bioinformatically detected regulatory, non protein coding, viral micro RNA (miRNA) gene. The method by which VGAM16 was detected is described hereinabove with reference to FIGS. 1-8.

VGAM16 gene, herein designated VGAM GENE, is a viral gene contained in the genome of Vaccinia Virus. VGAM16 host target gene, herein designated VGAM HOST TARGET GENE, is a human gene contained in the human genome.

VGAM16 gene encodes a VGAM16 precursor RNA, herein designated VGAM PRECURSOR RNA. Similar to other miRNA genes, and unlike most ordinary genes, VGAM16 precursor RNA does not encode a protein. A nucleotide sequence identical or highly similar to the nucleotide sequence of VGAM16 precursor RNA is designated SEQ ID:2, and is provided hereinbelow with reference to the sequence listing part. Nucleotide sequence SEQ ID:2 is located at position 188927 relative to the genome of Vaccinia Virus.

VGAM16 precursor RNA folds onto itself, forming VGAM16 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM16 folded precursor RNA into VGAM16 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM16 RNA is designated SEQ ID:351, and is provided hereinbelow with reference to the sequence listing part.

VGAM16 host target gene, herein designated VGAM HOST TARGET GENE, encodes a corresponding messenger RNA, VGAM16 host target RNA, herein designated VGAM HOST TARGET RNA. VGAM16 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM16 RNA, herein designated VGAM RNA, binds complementarily to one or more host target binding sites located in untranslated regions of VGAM16 host target RNA, herein designated VGAM HOST TARGET RNA. This complementary binding is due to the fact that the nucleotide sequence of VGAM16 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM16 RNA, herein designated VGAM RNA, may have a different number of host target binding sites in untranslated regions of a VGAM16 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM16 RNA, herein designated VGAM RNA, to host target binding sites on VGAM16 host target RNA, herein designated VGAM HOST TARGET RNA, such as BINDING SITE I, BINDING SITE II and BINDING SITE III, inhibits translation of VGAM16 host target RNA into VGAM16 host target protein, herein designated VGAM HOST TARGET PROTEIN. VGAM host target protein is therefore outlined by a broken line.

It is appreciated that VGAM16 host target gene, herein designated VGAM HOST TARGET GENE, in fact represents a plurality of VGAM16 host target genes. The mRNA of each one of this plurality of VGAM16 host target genes comprises one or more host target binding sites, each having a nucleotide sequence which is at least partly complementary to VGAM16 RNA, herein designated VGAM RNA, and which when bound by VGAM16 RNA causes inhibition of translation of respective one or more VGAM16 host target proteins.

It is further appreciated by one skilled in the art that the mode of translational inhibition illustrated by FIG. 1 with specific reference to translational inhibition exerted by VGAM16 gene, herein designated VGAM GENE, on one or more VGAM16 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM16 is inhibition of expression of host target genes, as part of a novel viral mechanism of attacking a host. Accordingly, utilities of VGAM16 include diagnosis, prevention and treatment of viral infection by Vaccinia Virus. Specific functions, and accordingly utilities, of VGAM16 correlate with, and may be deduced from, the identity of the host target genes which VGAM16 binds and inhibits, and the function of these host target genes, as elaborated hereinbelow.

Nucleotide sequences of the VGAM16 precursor RNA, herein designated VGAM PRECURSOR RNA, and of the ‘diced’ VGAM16 RNA, herein designated VGAM RNA, and a schematic representation of the secondary folding of VGAM16 folded precursor RNA, herein designated VGAM FOLDED PRECURSOR RNA, of VGAM16 are further described hereinbelow with reference to Table 1.

Nucleotide sequences of host target binding sites, such as BINDING SITE-I, BINDING SITE-II and BINDING SITE-III of FIG. 1, found on VGAM16 host target RNA, and schematic representation of the complementarity of each of these host target binding sites to VGAM16 RNA, herein designated VGAM RNA, are described hereinbelow with reference to Table 2.

As mentioned hereinabove with reference to FIG. 1, a function of VGAM16 gene, herein designated VGAM is inhibition of expression of VGAM16 target genes. It is appreciated that specific functions, and accordingly utilities, of VGAM16 correlate with, and may be deduced from, the identity of the target genes which VGAM16 binds and inhibits, and the function of these target genes, as elaborated hereinbelow.

Calpain 2, (m/II) Large Subunit (CAPN2, Accession NM_{—}001748) is a VGAM16 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM16 RNA, herein designated VGAM RNA, also designated SEQ ID:351.

A function of VGAM16 is therefore inhibition of Calpain 2, (m/II) Large Subunit (CAPN2, Accession NM_{—}001748). Accordingly, utilities of VGAM16 include diagnosis, prevention and treatment of diseases and clinical conditions associated with CAPN2. Dishevelled Associated Activator of Morphogenesis 2 (DAAM2, Accession XM_{—}166434) is another VGAM16 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM16 RNA, herein designated VGAM RNA, also designated SEQ ID:351.

Another function of VGAM16 is therefore inhibition of Dishevelled Associated Activator of Morphogenesis 2 (DAAM2, Accession XM_{—}166434), a gene which controls cell polarity and movement during development. Accordingly, utilities of VGAM16 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM16 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM16 RNA, herein designated VGAM RNA, also designated SEQ ID:351.

Another function of VGAM16 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM16 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM16 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM16 RNA, herein designated VGAM RNA, also designated SEQ ID:351.

Another function of VGAM16 is therefore inhibition of KIAA0475 (Accession NM_{—}014864). Accordingly, utilities of VGAM16 include diagnosis, prevention and treatment of diseases and clinical conditions associated with KIAA0475. LOC222671 (Accession XM_{—}167094) is another VGAM16 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM16 RNA, herein designated VGAM RNA, also designated SEQ ID:351.

Another function of VGAM16 is therefore inhibition of LOC222671 (Accession XM_{—}167094). Accordingly, utilities of VGAM16 include diagnosis, prevention and treatment of diseases and clinical conditions associated with LOC222671. LOC254901 (Accession XM_{—}171878) is another VGAM16 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM16 RNA, herein designated VGAM RNA, also designated SEQ ID:351.

Another function of VGAM16 is therefore inhibition of LOC254901 (Accession XM_{—}171878). Accordingly, utilities of VGAM16 include diagnosis, prevention and treatment of diseases and clinical conditions associated with LOC254901. LOC257615 (Accession XM_{—}175293) is another VGAM16 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM16 RNA, herein designated VGAM RNA, also designated SEQ ID:351.

Another function of VGAM16 is therefore inhibition of LOC257615 (Accession XM_{—}175293). Accordingly, utilities of VGAM16 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 17 (VGAM17) viral gene, which modulates expression of respective host target genes thereof, the function and utility of which host target genes is known in the art.

VGAM17 is a novel bioinformatically detected regulatory, non protein coding, viral micro RNA (miRNA) gene. The method by which VGAM17 was detected is described hereinabove with reference to FIGS. 1-8.

VGAM17 gene, herein designated VGAM GENE, is a viral gene contained in the genome of Vaccinia Virus. VGAM17 host target gene, herein designated VGAM HOST TARGET GENE, is a human gene contained in the human genome.

VGAM17 gene encodes a VGAM17 precursor RNA, herein designated VGAM PRECURSOR RNA. Similar to other miRNA genes, and unlike most ordinary genes, VGAM17 precursor RNA does not encode a protein. A nucleotide sequence identical or highly similar to the nucleotide sequence of VGAM17 precursor RNA is designated SEQ ID:3, and is provided hereinbelow with reference to the sequence listing part. Nucleotide sequence SEQ ID:3 is located at position 188927 relative to the genome of Vaccinia Virus.

VGAM17 precursor RNA folds onto itself, forming VGAM17 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM17 folded precursor RNA into VGAM17 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM17 RNA is designated SEQ ID:352, and is provided hereinbelow with reference to the sequence listing part.

VGAM17 host target gene, herein designated VGAM HOST TARGET GENE, encodes a corresponding messenger RNA, VGAM17 host target RNA, herein designated VGAM HOST TARGET RNA. VGAM17 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM17 RNA, herein designated VGAM RNA, binds complementarily to one or more host target binding sites located in untranslated regions of VGAM17 host target RNA, herein designated VGAM HOST TARGET RNA. This complementary binding is due to the fact that the nucleotide sequence of VGAM17 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM17 RNA, herein designated VGAM RNA, may have a different number of host target binding sites in untranslated regions of a VGAM17 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM17 RNA, herein designated VGAM RNA, to host target binding sites on VGAM17 host target RNA, herein designated VGAM HOST TARGET RNA, such as BINDING SITE I, BINDING SITE II and BINDING SITE III, inhibits translation of VGAM17 host target RNA into VGAM17 host target protein, herein designated VGAM HOST TARGET PROTEIN. VGAM host target protein is therefore outlined by a broken line.

It is appreciated that VGAM17 host target gene, herein designated VGAM HOST TARGET GENE, in fact represents a plurality of VGAM17 host target genes. The mRNA of each one of this plurality of VGAM17 host target genes comprises one or more host target binding sites, each having a nucleotide sequence which is at least partly complementary to VGAM17 RNA, herein designated VGAM RNA, and which when bound by VGAM17 RNA causes inhibition of translation of respective one or more VGAM17 host target proteins.

It is further appreciated by one skilled in the art that the mode of translational inhibition illustrated by FIG. 1 with specific reference to translational inhibition exerted by VGAM17 gene, herein designated VGAM GENE, on one or more VGAM17 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM17 is inhibition of expression of host target genes, as part of a novel viral mechanism of attacking a host. Accordingly, utilities of VGAM17 include diagnosis, prevention and treatment of viral infection by Vaccinia Virus. Specific functions, and accordingly utilities, of VGAM17 correlate with, and may be deduced from, the identity of the host target genes which VGAM17 binds and inhibits, and the function of these host target genes, as elaborated hereinbelow.

Nucleotide sequences of the VGAM17 precursor RNA, herein designated VGAM PRECURSOR RNA, and of the ‘diced’ VGAM17 RNA, herein designated VGAM RNA, and a schematic representation of the secondary folding of VGAM17 folded precursor RNA, herein designated VGAM FOLDED PRECURSOR RNA, of VGAM17 are further described hereinbelow with reference to Table 1.

Nucleotide sequences of host target binding sites, such as BINDING SITE-I, BINDING SITE-II and BINDING SITE-III of FIG. 1, found on VGAM17 host target RNA, and schematic representation of the complementarity of each of these host target binding sites to VGAM17 RNA, herein designated VGAM RNA, are described hereinbelow with reference to Table 2.

As mentioned hereinabove with reference to FIG. 1, a function of VGAM17 gene, herein designated VGAM is inhibition of expression of VGAM17 target genes. It is appreciated that specific functions, and accordingly utilities, of VGAM17 correlate with, and may be deduced from, the identity of the target genes which VGAM17 binds and inhibits, and the function of these target genes, as elaborated hereinbelow.

Calpain 2, (m/II) Large Subunit (CAPN2, Accession NM_{—}001748) is a VGAM17 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM17 RNA, herein designated VGAM RNA, also designated SEQ ID:352.

A function of VGAM17 is therefore inhibition of Calpain 2, (m/II) Large Subunit (CAPN2, Accession NM_{—}001748). Accordingly, utilities of VGAM17 include diagnosis, prevention and treatment of diseases and clinical conditions associated with CAPN2. Dishevelled Associated Activator of Morphogenesis 2 (DAAM2, Accession XM_{—}166434) is another VGAM17 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM17 RNA, herein designated VGAM RNA, also designated SEQ ID:352.

Another function of VGAM17 is therefore inhibition of Dishevelled Associated Activator of Morphogenesis 2 (DAAM2, Accession XM_{—}166434), a gene which controls cell polarity and movement during development. Accordingly, utilities of VGAM17 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM17 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM17 RNA, herein designated VGAM RNA, also designated SEQ ID:352.

Another function of VGAM17 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM17 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM17 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM17 RNA, herein designated VGAM RNA, also designated SEQ ID:352.

Another function of VGAM17 is therefore inhibition of KIAA0475 (Accession NM_{—}014864). Accordingly, utilities of VGAM17 include diagnosis, prevention and treatment of diseases and clinical conditions associated with KIAA0475. LOC222671 (Accession XM_{—}167094) is another VGAM17 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM17 RNA, herein designated VGAM RNA, also designated SEQ ID:352.

Another function of VGAM17 is therefore inhibition of LOC222671 (Accession XM_{—}167094). Accordingly, utilities of VGAM17 include diagnosis, prevention and treatment of diseases and clinical conditions associated with LOC222671. LOC254901 (Accession XM_{—}171878) is another VGAM17 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM17 RNA, herein designated VGAM RNA, also designated SEQ ID:352.

Another function of VGAM17 is therefore inhibition of LOC254901 (Accession XM_{—}171878). Accordingly, utilities of VGAM17 include diagnosis, prevention and treatment of diseases and clinical conditions associated with LOC254901. LOC257615 (Accession XM_{—}175293) is another VGAM17 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM17 RNA, herein designated VGAM RNA, also designated SEQ ID:352.

Another function of VGAM17 is therefore inhibition of LOC257615 (Accession XM_{—}175293). Accordingly, utilities of VGAM17 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 18 (VGAM18) viral gene, which modulates expression of respective host target genes thereof, the function and utility of which host target genes is known in the art.

VGAM18 is a novel bioinformatically detected regulatory, non protein coding, viral micro RNA (miRNA) gene. The method by which VGAM18 was detected is described hereinabove with reference to FIGS. 1-8.

VGAM18 gene, herein designated VGAM GENE, is a viral gene contained in the genome of Vaccinia Virus. VGAM18 host target gene, herein designated VGAM HOST TARGET GENE, is a human gene contained in the human genome.

VGAM18 gene encodes a VGAM18 precursor RNA, herein designated VGAM PRECURSOR RNA. Similar to other miRNA genes, and unlike most ordinary genes, VGAM18 precursor RNA does not encode a protein. A nucleotide sequence identical or highly similar to the nucleotide sequence of VGAM18 precursor RNA is designated SEQ ID:4, and is provided hereinbelow with reference to the sequence listing part. Nucleotide sequence SEQ ID:4 is located at position 188927 relative to the genome of Vaccinia Virus.

VGAM18 precursor RNA folds onto itself, forming VGAM18 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM18 folded precursor RNA into VGAM18 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM18 RNA is designated SEQ ID:353, and is provided hereinbelow with reference to the sequence listing part.

VGAM18 host target gene, herein designated VGAM HOST TARGET GENE, encodes a corresponding messenger RNA, VGAM18 host target RNA, herein designated VGAM HOST TARGET RNA. VGAM18 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM18 RNA, herein designated VGAM RNA, binds complementarily to one or more host target binding sites located in untranslated regions of VGAM18 host target RNA, herein designated VGAM HOST TARGET RNA. This complementary binding is due to the fact that the nucleotide sequence of VGAM18 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM18 RNA, herein designated VGAM RNA, may have a different number of host target binding sites in untranslated regions of a VGAM18 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM18 RNA, herein designated VGAM RNA, to host target binding sites on VGAM18 host target RNA, herein designated VGAM HOST TARGET RNA, such as BINDING SITE I, BINDING SITE II and BINDING SITE III, inhibits translation of VGAM18 host target RNA into VGAM18 host target protein, herein designated VGAM HOST TARGET PROTEIN. VGAM host target protein is therefore outlined by a broken line.

It is appreciated that VGAM18 host target gene, herein designated VGAM HOST TARGET GENE, in fact represents a plurality of VGAM18 host target genes. The mRNA of each one of this plurality of VGAM18 host target genes comprises one or more host target binding sites, each having a nucleotide sequence which is at least partly complementary to VGAM18 RNA, herein designated VGAM RNA, and which when bound by VGAM18 RNA causes inhibition of translation of respective one or more VGAM18 host target proteins.

It is further appreciated by one skilled in the art that the mode of translational inhibition illustrated by FIG. 1 with specific reference to translational inhibition exerted by VGAM18 gene, herein designated VGAM GENE, on one or more VGAM18 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM18 is inhibition of expression of host target genes, as part of a novel viral mechanism of attacking a host. Accordingly, utilities of VGAM18 include diagnosis, prevention and treatment of viral infection by Vaccinia Virus. Specific functions, and accordingly utilities, of VGAM18 correlate with, and may be deduced from, the identity of the host target genes which VGAM18 binds and inhibits, and the function of these host target genes, as elaborated hereinbelow.

Nucleotide sequences of the VGAM18 precursor RNA, herein designated VGAM PRECURSOR RNA, and of the ‘diced’ VGAM18 RNA, herein designated VGAM RNA, and a schematic representation of the secondary folding of VGAM18 folded precursor RNA, herein designated VGAM FOLDED PRECURSOR RNA, of VGAM18 are further described hereinbelow with reference to Table 1.

Nucleotide sequences of host target binding sites, such as BINDING SITE-I, BINDING SITE-II and BINDING SITE-III of FIG. 1, found on VGAM18 host target RNA, and schematic representation of the complementarity of each of these host target binding sites to VGAM18 RNA, herein designated VGAM RNA, are described hereinbelow with reference to Table 2.

As mentioned hereinabove with reference to FIG. 1, a function of VGAM18 gene, herein designated VGAM is inhibition of expression of VGAM18 target genes. It is appreciated that specific functions, and accordingly utilities, of VGAM18 correlate with, and may be deduced from, the identity of the target genes which VGAM18 binds and inhibits, and the function of these target genes, as elaborated hereinbelow.

Calpain 2, (m/II) Large Subunit (CAPN2, Accession NM_{—}001748) is a VGAM18 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM18 RNA, herein designated VGAM RNA, also designated SEQ ID:353.

A function of VGAM18 is therefore inhibition of Calpain 2, (m/II) Large Subunit (CAPN2, Accession NM_{—}001748). Accordingly, utilities of VGAM18 include diagnosis, prevention and treatment of diseases and clinical conditions associated with CAPN2. Dishevelled Associated Activator of Morphogenesis 2 (DAAM2, Accession XM_{—}166434) is another VGAM18 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM18 RNA, herein designated VGAM RNA, also designated SEQ ID:353.

Another function of VGAM18 is therefore inhibition of Dishevelled Associated Activator of Morphogenesis 2 (DAAM2, Accession XM_{—}166434), a gene which controls cell polarity and movement during development. Accordingly, utilities of VGAM18 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM18 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM18 RNA, herein designated VGAM RNA, also designated SEQ ID:353.

Another function of VGAM18 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM18 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM18 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM18 RNA, herein designated VGAM RNA, also designated SEQ ID:353.

Another function of VGAM18 is therefore inhibition of KIAA0475 (Accession NM_{—}014864). Accordingly, utilities of VGAM18 include diagnosis, prevention and treatment of diseases and clinical conditions associated with KIAA0475. LOC222671 (Accession XM_{—}167094) is another VGAM18 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM18 RNA, herein designated VGAM RNA, also designated SEQ ID:353.

Another function of VGAM18 is therefore inhibition of LOC222671 (Accession XM_{—}167094). Accordingly, utilities of VGAM18 include diagnosis, prevention and treatment of diseases and clinical conditions associated with LOC222671. LOC254901 (Accession XM_{—}171878) is another VGAM18 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM18 RNA, herein designated VGAM RNA, also designated SEQ ID:353.

Another function of VGAM18 is therefore inhibition of LOC254901 (Accession XM_{—}171878). Accordingly, utilities of VGAM18 include diagnosis, prevention and treatment of diseases and clinical conditions associated with LOC254901. LOC257615 (Accession XM_{—}175293) is another VGAM18 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM18 RNA, herein designated VGAM RNA, also designated SEQ ID:353.

Another function of VGAM18 is therefore inhibition of LOC257615 (Accession XM_{—}175293). Accordingly, utilities of VGAM18 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 19 (VGAM19) viral gene, which modulates expression of respective host target genes thereof, the function and utility of which host target genes is known in the art.

VGAM19 is a novel bioinformatically detected regulatory, non protein coding, viral micro RNA (miRNA) gene. The method by which VGAM19 was detected is described hereinabove with reference to FIGS. 1-8.

VGAM19 gene, herein designated VGAM GENE, is a viral gene contained in the genome of Vaccinia Virus. VGAM19 host target gene, herein designated VGAM HOST TARGET GENE, is a human gene contained in the human genome.

VGAM19 gene encodes a VGAM19 precursor RNA, herein designated VGAM PRECURSOR RNA. Similar to other miRNA genes, and unlike most ordinary genes, VGAM19 precursor RNA does not encode a protein. A nucleotide sequence identical or highly similar to the nucleotide sequence of VGAM19 precursor RNA is designated SEQ ID:5, and is provided hereinbelow with reference to the sequence listing part. Nucleotide sequence SEQ ID:5 is located at position 188927 relative to the genome of Vaccinia Virus.

VGAM19 precursor RNA folds onto itself, forming VGAM19 folded precursor RNA, herein designated VGAM FOLDED PRECURSOR RNA, which has a two-dimensional “hairpin structure”. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM19 folded precursor RNA into VGAM19 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM19 RNA is designated SEQ ID:354, and is provided hereinbelow with reference to the sequence listing part.

VGAM19 host target gene, herein designated VGAM HOST TARGET GENE, encodes a corresponding messenger RNA, VGAM19 host target RNA, herein designated VGAM HOST TARGET RNA. VGAM19 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM19 RNA, herein designated VGAM RNA, binds complementarily to one or more host target binding sites located in untranslated regions of VGAM19 host target RNA, herein designated VGAM HOST TARGET RNA. This complementary binding is due to the fact that the nucleotide sequence of VGAM19 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM19 RNA, herein designated VGAM RNA, may have a different number of host target binding sites in untranslated regions of a VGAM19 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′ UTR and 5′UTR regions.

The complementary binding of VGAM19 RNA, herein designated VGAM RNA, to host target binding sites on VGAM19 host target RNA, herein designated VGAM HOST TARGET RNA, such as BINDING SITE I, BINDING SITE II and BINDING SITE III, inhibits translation of VGAM19 host target RNA into VGAM19 host target protein, herein designated VGAM HOST TARGET PROTEIN. VGAM host target protein is therefore outlined by a broken line.

It is appreciated that VGAM19 host target gene, herein designated VGAM HOST TARGET GENE, in fact represents a plurality of VGAM19 host target genes. The mRNA of each one of this plurality of VGAM19 host target genes comprises one or more host target binding sites, each having a nucleotide sequence which is at least partly complementary to VGAM19 RNA, herein designated VGAM RNA, and which when bound by VGAM19 RNA causes inhibition of translation of respective one or more VGAM19 host target proteins.

It is further appreciated by one skilled in the art that the mode of translational inhibition illustrated by FIG. 1 with specific reference to translational inhibition exerted by VGAM19 gene, herein designated VGAM GENE, on one or more VGAM19 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM19 is inhibition of expression of host target genes, as part of a novel viral mechanism of attacking a host. Accordingly, utilities of VGAM19 include diagnosis, prevention and treatment of viral infection by Vaccinia Virus. Specific functions, and accordingly utilities, of VGAM19 correlate with, and may be deduced from, the identity of the host target genes which VGAM19 binds and inhibits, and the function of these host target genes, as elaborated hereinbelow.

Nucleotide sequences of the VGAM19 precursor RNA, herein designated VGAM PRECURSOR RNA, and of the ‘diced’ VGAM19 RNA, herein designated VGAM RNA, and a schematic representation of the secondary folding of VGAM19 folded precursor RNA, herein designated VGAM FOLDED PRECURSOR RNA, of VGAM19 are further described hereinbelow with reference to Table 1.

Nucleotide sequences of host target binding sites, such as BINDING SITE-I, BINDING SITE-II and BINDING SITE-III of FIG. 1, found on VGAM19 host target RNA, and schematic representation of the complementarity of each of these host target binding sites to VGAM19 RNA, herein designated VGAM RNA, are described hereinbelow with reference to Table 2.

As mentioned hereinabove with reference to FIG. 1, a function of VGAM19 gene, herein designated VGAM is inhibition of expression of VGAM19 target genes. It is appreciated that specific functions, and accordingly utilities, of VGAM19 correlate with, and may be deduced from, the identity of the target genes which VGAM19 binds and inhibits, and the function of these target genes, as elaborated hereinbelow.

Calpain 2, (m/II) Large Subunit (CAPN2, Accession NM_{—}001748) is a VGAM19 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM19 RNA, herein designated VGAM RNA, also designated SEQ ID:354.

A function of VGAM19 is therefore inhibition of Calpain 2, (m/II) Large Subunit (CAPN2, Accession NM_{—}001748). Accordingly, utilities of VGAM19 include diagnosis, prevention and treatment of diseases and clinical conditions associated with CAPN2. Dishevelled Associated Activator of Morphogenesis 2 (DAAM2, Accession XM_{—}166434) is another VGAM19 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM19 RNA, herein designated VGAM RNA, also designated SEQ ID:354.

Another function of VGAM19 is therefore inhibition of Dishevelled Associated Activator of Morphogenesis 2 (DAAM2, Accession XM_{—}166434), a gene which controls cell polarity and movement during development. Accordingly, utilities of VGAM19 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM19 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM19 RNA, herein designated VGAM RNA, also designated SEQ ID:354.

Another function of VGAM19 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM19 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM19 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM19 RNA, herein designated VGAM RNA, also designated SEQ ID:354.

Another function of VGAM19 is therefore inhibition of KIAA0475 (Accession NM_{—}014864). Accordingly, utilities of VGAM19 include diagnosis, prevention and treatment of diseases and clinical conditions associated with KIAA0475. LOC222671 (Accession XM_{—}167094) is another VGAM19 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM19 RNA, herein designated VGAM RNA, also designated SEQ ID:354.

Another function of VGAM19 is therefore inhibition of LOC222671 (Accession XM_{—}167094). Accordingly, utilities of VGAM19 include diagnosis, prevention and treatment of diseases and clinical conditions associated with LOC222671. LOC254901 (Accession XM_{—}171878) is another VGAM19 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM19 RNA, herein designated VGAM RNA, also designated SEQ ID:354.

Another function of VGAM19 is therefore inhibition of LOC254901 (Accession XM_{—}171878). Accordingly, utilities of VGAM19 include diagnosis, prevention and treatment of diseases and clinical conditions associated with LOC254901. LOC257615 (Accession XM_{—}175293) is another VGAM19 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM19 RNA, herein designated VGAM RNA, also designated SEQ ID:354.

Another function of VGAM19 is therefore inhibition of LOC257615 (Accession XM_{—}175293). Accordingly, utilities of VGAM19 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 20 (VGAM20) viral gene, which modulates expression of respective host target genes thereof, the function and utility of which host target genes is known in the art.

VGAM20 is a novel bioinformatically detected regulatory, non protein coding, viral micro RNA (miRNA) gene. The method by which VGAM20 was detected is described hereinabove with reference to FIGS. 1-8.

VGAM20 gene, herein designated VGAM GENE, is a viral gene contained in the genome of Vaccinia Virus. VGAM20 host target gene, herein designated VGAM HOST TARGET GENE, is a human gene contained in the human genome.

VGAM20 gene encodes a VGAM20 precursor RNA, herein designated VGAM PRECURSOR RNA. Similar to other miRNA genes, and unlike most ordinary genes, VGAM20 precursor RNA does not encode a protein. A nucleotide sequence identical or highly similar to the nucleotide sequence of VGAM20 precursor RNA is designated SEQ ID:6, and is provided hereinbelow with reference to the sequence listing part. Nucleotide sequence SEQ ID:6 is located at position 188927 relative to the genome of Vaccinia Virus.

VGAM20 precursor RNA folds onto itself, forming VGAM20 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM20 folded precursor RNA into VGAM20 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM20 RNA is designated SEQ ID:355, and is provided hereinbelow with reference to the sequence listing part.

VGAM20 host target gene, herein designated VGAM HOST TARGET GENE, encodes a corresponding messenger RNA, VGAM20 host target RNA, herein designated VGAM HOST TARGET RNA. VGAM20 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM20 RNA, herein designated VGAM RNA, binds complementarily to one or more host target binding sites located in untranslated regions of VGAM20 host target RNA, herein designated VGAM HOST TARGET RNA. This complementary binding is due to the fact that the nucleotide sequence of VGAM20 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM20 RNA, herein designated VGAM RNA, may have a different number of host target binding sites in untranslated regions of a VGAM20 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM20 RNA, herein designated VGAM RNA, to host target binding sites on VGAM20 host target RNA, herein designated VGAM HOST TARGET RNA, such as BINDING SITE I, BINDING SITE II and BINDING SITE III, inhibits translation of VGAM20 host target RNA into VGAM20 host target protein, herein designated VGAM HOST TARGET PROTEIN. VGAM host target protein is therefore outlined by a broken line.

It is appreciated that VGAM20 host target gene, herein designated VGAM HOST TARGET GENE, in fact represents a plurality of VGAM20 host target genes. The mRNA of each one of this plurality of VGAM20 host target genes comprises one or more host target binding sites, each having a nucleotide sequence which is at least partly complementary to VGAM20 RNA, herein designated VGAM RNA, and which when bound by VGAM20 RNA causes inhibition of translation of respective one or more VGAM20 host target proteins.

It is further appreciated by one skilled in the art that the mode of translational inhibition illustrated by FIG. 1 with specific reference to translational inhibition exerted by VGAM20 gene, herein designated VGAM GENE, on one or more VGAM20 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM20 is inhibition of expression of host target genes, as part of a novel viral mechanism of attacking a host. Accordingly, utilities of VGAM20 include diagnosis, prevention and treatment of viral infection by Vaccinia Virus. Specific functions, and accordingly utilities, of VGAM20 correlate with, and may be deduced from, the identity of the host target genes which VGAM20 binds and inhibits, and the function of these host target genes, as elaborated hereinbelow.

Nucleotide sequences of the VGAM20 precursor RNA, herein designated VGAM PRECURSOR RNA, and of the ‘diced’ VGAM20 RNA, herein designated VGAM RNA, and a schematic representation of the secondary folding of VGAM20 folded precursor RNA, herein designated VGAM FOLDED PRECURSOR RNA, of VGAM20 are further described hereinbelow with reference to Table 1.

Nucleotide sequences of host target binding sites, such as BINDING SITE-I, BINDING SITE-II and BINDING SITE-III of FIG. 1, found on VGAM20 host target RNA, and schematic representation of the complementarity of each of these host target binding sites to VGAM20 RNA, herein designated VGAM RNA, are described hereinbelow with reference to Table 2.

As mentioned hereinabove with reference to FIG. 1, a function of VGAM20 gene, herein designated VGAM is inhibition of expression of VGAM20 target genes. It is appreciated that specific functions, and accordingly utilities, of VGAM20 correlate with, and may be deduced from, the identity of the target genes which VGAM20 binds and inhibits, and the function of these target genes, as elaborated hereinbelow.

Calpain 2, (m/II) Large Subunit (CAPN2, Accession NM_{—}001748) is a VGAM20 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM20 RNA, herein designated VGAM RNA, also designated SEQ ID:355.

A function of VGAM20 is therefore inhibition of Calpain 2, (m/II) Large Subunit (CAPN2, Accession NM_{—}001748). Accordingly, utilities of VGAM20 include diagnosis, prevention and treatment of diseases and clinical conditions associated with CAPN2. Dishevelled Associated Activator of Morphogenesis 2 (DAAM2, Accession XM_{—}166434) is another VGAM20 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM20 RNA, herein designated VGAM RNA, also designated SEQ ID:355.

Another function of VGAM20 is therefore inhibition of Dishevelled Associated Activator of Morphogenesis 2 (DAAM2, Accession XM_{—}166434), a gene which controls cell polarity and movement during development. Accordingly, utilities of VGAM20 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM20 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM20 RNA, herein designated VGAM RNA, also designated SEQ ID:355.

Another function of VGAM20 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM20 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM20 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM20 RNA, herein designated VGAM RNA, also designated SEQ ID:355.

Another function of VGAM20 is therefore inhibition of KIAA0475 (Accession NM_{—}014864). Accordingly, utilities of VGAM20 include diagnosis, prevention and treatment of diseases and clinical conditions associated with KIAA0475. LOC222671 (Accession XM_{—}167094) is another VGAM20 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM20 RNA, herein designated VGAM RNA, also designated SEQ ID:355.

Another function of VGAM20 is therefore inhibition of LOC222671 (Accession XM_{—}167094). Accordingly, utilities of VGAM20 include diagnosis, prevention and treatment of diseases and clinical conditions associated with LOC222671. LOC254901 (Accession XM_{—}171878) is another VGAM20 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM20 RNA, herein designated VGAM RNA, also designated SEQ ID:355.

Another function of VGAM20 is therefore inhibition of LOC254901 (Accession XM_{—}171878). Accordingly, utilities of VGAM20 include diagnosis, prevention and treatment of diseases and clinical conditions associated with LOC254901. LOC257615 (Accession XM_{—}175293) is another VGAM20 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM20 RNA, herein designated VGAM RNA, also designated SEQ ID:355.

Another function of VGAM20 is therefore inhibition of LOC257615 (Accession XM_{—}175293). Accordingly, utilities of VGAM20 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 21 (VGAM21) viral gene, which modulates expression of respective host target genes thereof, the function and utility of which host target genes is known in the art.

VGAM21 is a novel bioinformatically detected regulatory, non protein coding, viral micro RNA (miRNA) gene. The method by which VGAM21 was detected is described hereinabove with reference to FIGS. 1-8.

VGAM21 gene, herein designated VGAM GENE, is a viral gene contained in the genome of Vaccinia Virus. VGAM21 host target gene, herein designated VGAM HOST TARGET GENE, is a human gene contained in the human genome.

VGAM21 gene encodes a VGAM21 precursor RNA, herein designated VGAM PRECURSOR RNA. Similar to other miRNA genes, and unlike most ordinary genes, VGAM21 precursor RNA does not encode a protein. A nucleotide sequence identical or highly similar to the nucleotide sequence of VGAM21 precursor RNA is designated SEQ ID:7, and is provided hereinbelow with reference to the sequence listing part. Nucleotide sequence SEQ ID:7 is located at position 188927 relative to the genome of Vaccinia Virus.

VGAM21 precursor RNA folds onto itself, forming VGAM21 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM21 folded precursor RNA into VGAM21 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM21 RNA is designated SEQ ID:356, and is provided hereinbelow with reference to the sequence listing part.

VGAM21 host target gene, herein designated VGAM HOST TARGET GENE, encodes a corresponding messenger RNA, VGAM21 host target RNA, herein designated VGAM HOST TARGET RNA. VGAM21 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM21 RNA, herein designated VGAM RNA, binds complementarily to one or more host target binding sites located in untranslated regions of VGAM21 host target RNA, herein designated VGAM HOST TARGET RNA. This complementary binding is due to the fact that the nucleotide sequence of VGAM21 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM21 RNA, herein designated VGAM RNA, may have a different number of host target binding sites in untranslated regions of a VGAM21 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM21 RNA, herein designated VGAM RNA, to host target binding sites on VGAM21 host target RNA, herein designated VGAM HOST TARGET RNA, such as BINDING SITE I, BINDING SITE II and BINDING SITE III, inhibits translation of VGAM21 host target RNA into VGAM21 host target protein, herein designated VGAM HOST TARGET PROTEIN. VGAM host target protein is therefore outlined by a broken line.

It is appreciated that VGAM21 host target gene, herein designated VGAM HOST TARGET GENE, in fact represents a plurality of VGAM21 host target genes. The mRNA of each one of this plurality of VGAM21 host target genes comprises one or more host target binding sites, each having a nucleotide sequence which is at least partly complementary to VGAM21 RNA, herein designated VGAM RNA, and which when bound by VGAM21 RNA causes inhibition of translation of respective one or more VGAM21 host target proteins.

It is further appreciated by one skilled in the art that the mode of translational inhibition illustrated by FIG. 1 with specific reference to translational inhibition exerted by VGAM21 gene, herein designated VGAM GENE, on one or more VGAM21 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM21 is inhibition of expression of host target genes, as part of a novel viral mechanism of attacking a host. Accordingly, utilities of VGAM21 include diagnosis, prevention and treatment of viral infection by Vaccinia Virus. Specific functions, and accordingly utilities, of VGAM21 correlate with, and may be deduced from, the identity of the host target genes which VGAM21 binds and inhibits, and the function of these host target genes, as elaborated hereinbelow.

Nucleotide sequences of the VGAM21 precursor RNA, herein designated VGAM PRECURSOR RNA, and of the ‘diced’ VGAM21 RNA, herein designated VGAM RNA, and a schematic representation of the secondary folding of VGAM21 folded precursor RNA, herein designated VGAM FOLDED PRECURSOR RNA, of VGAM21 are further described hereinbelow with reference to Table 1.

Nucleotide sequences of host target binding sites, such as BINDING SITE-I, BINDING SITE-II and BINDING SITE-III of FIG. 1, found on VGAM21 host target RNA, and schematic representation of the complementarity of each of these host target binding sites to VGAM21 RNA, herein designated VGAM RNA, are described hereinbelow with reference to Table 2.

As mentioned hereinabove with reference to FIG. 1, a function of VGAM21 gene, herein designated VGAM is inhibition of expression of VGAM21 target genes. It is appreciated that specific functions, and accordingly utilities, of VGAM21 correlate with, and may be deduced from, the identity of the target genes which VGAM21 binds and inhibits, and the function of these target genes, as elaborated hereinbelow.

Calpain 2, (m/II) Large Subunit (CAPN2, Accession NM_{—}001748) is a VGAM21 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM21 RNA, herein designated VGAM RNA, also designated SEQ ID:356.

A function of VGAM21 is therefore inhibition of Calpain 2, (m/II) Large Subunit (CAPN2, Accession NM_{—}001748). Accordingly, utilities of VGAM21 include diagnosis, prevention and treatment of diseases and clinical conditions associated with CAPN2. Dishevelled Associated Activator of Morphogenesis 2 (DAAM2, Accession XM_{—}166434) is another VGAM21 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM21 RNA, herein designated VGAM RNA, also designated SEQ ID:356.

Another function of VGAM21 is therefore inhibition of Dishevelled Associated Activator of Morphogenesis 2 (DAAM2, Accession XM_{—}166434), a gene which controls cell polarity and movement during development. Accordingly, utilities of VGAM21 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM21 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM21 RNA, herein designated VGAM RNA, also designated SEQ ID:356.

Another function of VGAM21 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM21 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM21 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM21 RNA, herein designated VGAM RNA, also designated SEQ ID:356.

Another function of VGAM21 is therefore inhibition of KIAA0475 (Accession NM_{—}014864). Accordingly, utilities of VGAM21 include diagnosis, prevention and treatment of diseases and clinical conditions associated with KIAA0475. LOC222671 (Accession XM_{—}167094) is another VGAM21 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM21 RNA, herein designated VGAM RNA, also designated SEQ ID:356.

Another function of VGAM21 is therefore inhibition of LOC222671 (Accession XM_{—}167094). Accordingly, utilities of VGAM21 include diagnosis, prevention and treatment of diseases and clinical conditions associated with LOC222671. LOC254901 (Accession XM_{—}171878) is another VGAM21 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM21 RNA, herein designated VGAM RNA, also designated SEQ ID:356.

Another function of VGAM21 is therefore inhibition of LOC254901 (Accession XM_{—}171878). Accordingly, utilities of VGAM21 include diagnosis, prevention and treatment of diseases and clinical conditions associated with LOC254901. LOC257615 (Accession XM_{—}175293) is another VGAM21 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM21 RNA, herein designated VGAM RNA, also designated SEQ ID:356.

Another function of VGAM21 is therefore inhibition of LOC257615 (Accession XM_{—}175293). Accordingly, utilities of VGAM21 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 22 (VGAM22) viral gene, which modulates expression of respective host target genes thereof, the function and utility of which host target genes is known in the art.

VGAM22 is a novel bioinformatically detected regulatory, non protein coding, viral micro RNA (miRNA) gene. The method by which VGAM22 was detected is described hereinabove with reference to FIGS. 1-8.

VGAM22 gene, herein designated VGAM GENE, is a viral gene contained in the genome of Vaccinia Virus. VGAM22 host target gene, herein designated VGAM HOST TARGET GENE, is a human gene contained in the human genome.

VGAM22 gene encodes a VGAM22 precursor RNA, herein designated VGAM PRECURSOR RNA. Similar to other miRNA genes, and unlike most ordinary genes, VGAM22 precursor RNA does not encode a protein. A nucleotide sequence identical or highly similar to the nucleotide sequence of VGAM22 precursor RNA is designated SEQ ID:8, and is provided hereinbelow with reference to the sequence listing part. Nucleotide sequence SEQ ID:8 is located at position 188927 relative to the genome of Vaccinia Virus.

VGAM22 precursor RNA folds onto itself, forming VGAM22 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM22 folded precursor RNA into VGAM22 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM22 RNA is designated SEQ ID:357, and is provided hereinbelow with reference to the sequence listing part.

VGAM22 host target gene, herein designated VGAM HOST TARGET GENE, encodes a corresponding messenger RNA, VGAM22 host target RNA, herein designated VGAM HOST TARGET RNA. VGAM22 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM22 RNA, herein designated VGAM RNA, binds complementarily to one or more host target binding sites located in untranslated regions of VGAM22 host target RNA, herein designated VGAM HOST TARGET RNA. This complementary binding is due to the fact that the nucleotide sequence of VGAM22 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM22 RNA, herein designated VGAM RNA, may have a different number of host target binding sites in untranslated regions of a VGAM22 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM22 RNA, herein designated VGAM RNA, to host target binding sites on VGAM22 host target RNA, herein designated VGAM HOST TARGET RNA, such as BINDING SITE I, BINDING SITE II and BINDING SITE III, inhibits translation of VGAM22 host target RNA into VGAM22 host target protein, herein designated VGAM HOST TARGET PROTEIN. VGAM host target protein is therefore outlined by a broken line.

It is appreciated that VGAM22 host target gene, herein designated VGAM HOST TARGET GENE, in fact represents a plurality of VGAM22 host target genes. The mRNA of each one of this plurality of VGAM22 host target genes comprises one or more host target binding sites, each having a nucleotide sequence which is at least partly complementary to VGAM22 RNA, herein designated VGAM RNA, and which when bound by VGAM22 RNA causes inhibition of translation of respective one or more VGAM22 host target proteins.

It is further appreciated by one skilled in the art that the mode of translational inhibition illustrated by FIG. 1 with specific reference to translational inhibition exerted by VGAM22 gene, herein designated VGAM GENE, on one or more VGAM22 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM22 is inhibition of expression of host target genes, as part of a novel viral mechanism of attacking a host. Accordingly, utilities of VGAM22 include diagnosis, prevention and treatment of viral infection by Vaccinia Virus. Specific functions, and accordingly utilities, of VGAM22 correlate with, and may be deduced from, the identity of the host target genes which VGAM22 binds and inhibits, and the function of these host target genes, as elaborated hereinbelow.

Nucleotide sequences of the VGAM22 precursor RNA, herein designated VGAM PRECURSOR RNA, and of the ‘diced’ VGAM22 RNA, herein designated VGAM RNA, and a schematic representation of the secondary folding of VGAM22 folded precursor RNA, herein designated VGAM FOLDED PRECURSOR RNA, of VGAM22 are further described hereinbelow with reference to Table 1.

Nucleotide sequences of host target binding sites, such as BINDING SITE-I, BINDING SITE-II and BINDING SITE-III of FIG. 1, found on VGAM22 host target RNA, and schematic representation of the complementarity of each of these host target binding sites to VGAM22 RNA, herein designated VGAM RNA, are described hereinbelow with reference to Table 2.

As mentioned hereinabove with reference to FIG. 1, a function of VGAM22 gene, herein designated VGAM is inhibition of expression of VGAM22 target genes. It is appreciated that specific functions, and accordingly utilities, of VGAM22 correlate with, and may be deduced from, the identity of the target genes which VGAM22 binds and inhibits, and the function of these target genes, as elaborated hereinbelow.

Calpain 2, (m/II) Large Subunit (CAPN2, Accession NM_{—}001748) is a VGAM22 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM22 RNA, herein designated VGAM RNA, also designated SEQ ID:357.

A function of VGAM22 is therefore inhibition of Calpain 2, (m/II) Large Subunit (CAPN2, Accession NM_{—}001748). Accordingly, utilities of VGAM22 include diagnosis, prevention and treatment of diseases and clinical conditions associated with CAPN2. Dishevelled Associated Activator of Morphogenesis 2 (DAAM2, Accession XM_{—}166434) is another VGAM22 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM22 RNA, herein designated VGAM RNA, also designated SEQ ID:357.

Another function of VGAM22 is therefore inhibition of Dishevelled Associated Activator of Morphogenesis 2 (DAAM2, Accession XM_{—}166434), a gene which controls cell polarity and movement during development. Accordingly, utilities of VGAM22 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM22 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM22 RNA, herein designated VGAM RNA, also designated SEQ ID:357.

Another function of VGAM22 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM22 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM22 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM22 RNA, herein designated VGAM RNA, also designated SEQ ID:357.

Another function of VGAM22 is therefore inhibition of KIAA0475 (Accession NM_{—}014864). Accordingly, utilities of VGAM22 include diagnosis, prevention and treatment of diseases and clinical conditions associated with KIAA0475. LOC222671 (Accession XM_{—}167094) is another VGAM22 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM22 RNA, herein designated VGAM RNA, also designated SEQ ID:357.

Another function of VGAM22 is therefore inhibition of LOC222671 (Accession XM_{—}167094). Accordingly, utilities of VGAM22 include diagnosis, prevention and treatment of diseases and clinical conditions associated with LOC222671. LOC254901 (Accession XM_{—}171878) is another VGAM22 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM22 RNA, herein designated VGAM RNA, also designated SEQ ID:357.

Another function of VGAM22 is therefore inhibition of LOC254901 (Accession XM_{—}171878). Accordingly, utilities of VGAM22 include diagnosis, prevention and treatment of diseases and clinical conditions associated with LOC254901. LOC257615 (Accession XM_{—}175293) is another VGAM22 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM22 RNA, herein designated VGAM RNA, also designated SEQ ID:357.

Another function of VGAM22 is therefore inhibition of LOC257615 (Accession XM_{—}175293). Accordingly, utilities of VGAM22 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 23 (VGAM23) viral gene, which modulates expression of respective host target genes thereof, the function and utility of which host target genes is known in the art.

VGAM23 is a novel bioinformatically detected regulatory, non protein coding, viral micro RNA (miRNA) gene. The method by which VGAM23 was detected is described hereinabove with reference to FIGS. 1-8.

VGAM23 gene, herein designated VGAM GENE, is a viral gene contained in the genome of Vaccinia Virus. VGAM23 host target gene, herein designated VGAM HOST TARGET GENE, is a human gene contained in the human genome.

VGAM23 gene encodes a VGAM23 precursor RNA, herein designated VGAM PRECURSOR RNA. Similar to other miRNA genes, and unlike most ordinary genes, VGAM23 precursor RNA does not encode a protein. A nucleotide sequence identical or highly similar to the nucleotide sequence of VGAM23 precursor RNA is designated SEQ ID:9, and is provided hereinbelow with reference to the sequence listing part. Nucleotide sequence SEQ ID:9 is located at position 188927 relative to the genome of Vaccinia Virus.

VGAM23 precursor RNA folds onto itself, forming VGAM23 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM23 folded precursor RNA into VGAM23 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM23 RNA is designated SEQ ID:358, and is provided hereinbelow with reference to the sequence listing part.

VGAM23 host target gene, herein designated VGAM HOST TARGET GENE, encodes a corresponding messenger RNA, VGAM23 host target RNA, herein designated VGAM HOST TARGET RNA. VGAM23 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM23 RNA, herein designated VGAM RNA, binds complementarily to one or more host target binding sites located in untranslated regions of VGAM23 host target RNA, herein designated VGAM HOST TARGET RNA. This complementary binding is due to the fact that the nucleotide sequence of VGAM23 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM23 RNA, herein designated VGAM RNA, may have a different number of host target binding sites in untranslated regions of a VGAM23 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM23 RNA, herein designated VGAM RNA, to host target binding sites on VGAM23 host target RNA, herein designated VGAM HOST TARGET RNA, such as BINDING SITE I, BINDING SITE II and BINDING SITE III, inhibits translation of VGAM23 host target RNA into VGAM23 host target protein, herein designated VGAM HOST TARGET PROTEIN. VGAM host target protein is therefore outlined by a broken line.

It is appreciated that VGAM23 host target gene, herein designated VGAM HOST TARGET GENE, in fact represents a plurality of VGAM23 host target genes. The mRNA of each one of this plurality of VGAM23 host target genes comprises one or more host target binding sites, each having a nucleotide sequence which is at least partly complementary to VGAM23 RNA, herein designated VGAM RNA, and which when bound by VGAM23 RNA causes inhibition of translation of respective one or more VGAM23 host target proteins.

It is further appreciated by one skilled in the art that the mode of translational inhibition illustrated by FIG. 1 with specific reference to translational inhibition exerted by VGAM23 gene, herein designated VGAM GENE, on one or more VGAM23 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM23 is inhibition of expression of host target genes, as part of a novel viral mechanism of attacking a host. Accordingly, utilities of VGAM23 include diagnosis, prevention and treatment of viral infection by Vaccinia Virus. Specific functions, and accordingly utilities, of VGAM23 correlate with, and may be deduced from, the identity of the host target genes which VGAM23 binds and inhibits, and the function of these host target genes, as elaborated hereinbelow.

Nucleotide sequences of the VGAM23 precursor RNA, herein designated VGAM PRECURSOR RNA, and of the ‘diced’ VGAM23 RNA, herein designated VGAM RNA, and a schematic representation of the secondary folding of VGAM23 folded precursor RNA, herein designated VGAM FOLDED PRECURSOR RNA, of VGAM23 are further described hereinbelow with reference to Table 1.

Nucleotide sequences of host target binding sites, such as BINDING SITE-I, BINDING SITE-II and BINDING SITE-III of FIG. 1, found on VGAM23 host target RNA, and schematic representation of the complementarity of each of these host target binding sites to VGAM23 RNA, herein designated VGAM RNA, are described hereinbelow with reference to Table 2.

As mentioned hereinabove with reference to FIG. 1, a function of VGAM23 gene, herein designated VGAM is inhibition of expression of VGAM23 target genes. It is appreciated that specific functions, and accordingly utilities, of VGAM23 correlate with, and may be deduced from, the identity of the target genes which VGAM23 binds and inhibits, and the function of these target genes, as elaborated hereinbelow.

Calpain 2, (m/II) Large Subunit (CAPN2, Accession NM_{—}001748) is a VGAM23 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM23 RNA, herein designated VGAM RNA, also designated SEQ ID:358.

A function of VGAM23 is therefore inhibition of Calpain 2, (m/II) Large Subunit (CAPN2, Accession NM_{—}001748). Accordingly, utilities of VGAM23 include diagnosis, prevention and treatment of diseases and clinical conditions associated with CAPN2. Dishevelled Associated Activator of Morphogenesis 2 (DAAM2, Accession XM_{—}166434) is another VGAM23 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM23 RNA, herein designated VGAM RNA, also designated SEQ ID:358.

Another function of VGAM23 is therefore inhibition of Dishevelled Associated Activator of Morphogenesis 2 (DAAM2, Accession XM_{—}166434), a gene which controls cell polarity and movement during development. Accordingly, utilities of VGAM23 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM23 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM23 RNA, herein designated VGAM RNA, also designated SEQ ID:358.

Another function of VGAM23 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM23 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM23 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM23 RNA, herein designated VGAM RNA, also designated SEQ ID:358.

Another function of VGAM23 is therefore inhibition of KIAA0475 (Accession NM_{—}014864). Accordingly, utilities of VGAM23 include diagnosis, prevention and treatment of diseases and clinical conditions associated with KIAA0475. LOC222671 (Accession XM_{—}167094) is another VGAM23 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM23 RNA, herein designated VGAM RNA, also designated SEQ ID:358.

Another function of VGAM23 is therefore inhibition of LOC222671 (Accession XM_{—}167094). Accordingly, utilities of VGAM23 include diagnosis, prevention and treatment of diseases and clinical conditions associated with LOC222671. LOC254901 (Accession XM_{—}171878) is another VGAM23 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM23 RNA, herein designated VGAM RNA, also designated SEQ ID:358.

Another function of VGAM23 is therefore inhibition of LOC254901 (Accession XM_{—}171878). Accordingly, utilities of VGAM23 include diagnosis, prevention and treatment of diseases and clinical conditions associated with LOC254901. LOC257615 (Accession XM_{—}175293) is another VGAM23 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM23 RNA, herein designated VGAM RNA, also designated SEQ ID:358.

Another function of VGAM23 is therefore inhibition of LOC257615 (Accession XM_{—}175293). Accordingly, utilities of VGAM23 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 24 (VGAM24) viral gene, which modulates expression of respective host target genes thereof, the function and utility of which host target genes is known in the art.

VGAM24 is a novel bioinformatically detected regulatory, non protein coding, viral micro RNA (miRNA) gene. The method by which VGAM24 was detected is described hereinabove with reference to FIGS. 1-8.

VGAM24 gene, herein designated VGAM GENE, is a viral gene contained in the genome of Vaccinia Virus. VGAM24 host target gene, herein designated VGAM HOST TARGET GENE, is a human gene contained in the human genome.

VGAM24 gene encodes a VGAM24 precursor RNA, herein designated VGAM PRECURSOR RNA. Similar to other miRNA genes, and unlike most ordinary genes, VGAM24 precursor RNA does not encode a protein. A nucleotide sequence identical or highly similar to the nucleotide sequence of VGAM24 precursor RNA is designated SEQ ID:10, and is provided hereinbelow with reference to the sequence listing part. Nucleotide sequence SEQ ID: 10 is located at position 188927 relative to the genome of Vaccinia Virus.

VGAM24 precursor RNA folds onto itself, forming VGAM24 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM24 folded precursor RNA into VGAM24 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM24 RNA is designated SEQ ID:359, and is provided hereinbelow with reference to the sequence listing part.

VGAM24 host target gene, herein designated VGAM HOST TARGET GENE, encodes a corresponding messenger RNA, VGAM24 host target RNA, herein designated VGAM HOST TARGET RNA. VGAM24 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM24 RNA, herein designated VGAM RNA, binds complementarily to one or more host target binding sites located in untranslated regions of VGAM24 host target RNA, herein designated VGAM HOST TARGET RNA. This complementary binding is due to the fact that the nucleotide sequence of VGAM24 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM24 RNA, herein designated VGAM RNA, may have a different number of host target binding sites in untranslated regions of a VGAM24 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM24 RNA, herein designated VGAM RNA, to host target binding sites on VGAM24 host target RNA, herein designated VGAM HOST TARGET RNA, such as BINDING SITE I, BINDING SITE II and BINDING SITE III, inhibits translation of VGAM24 host target RNA into VGAM24 host target protein, herein designated VGAM HOST TARGET PROTEIN. VGAM host target protein is therefore outlined by a broken line.

It is appreciated that VGAM24 host target gene, herein designated VGAM HOST TARGET GENE, in fact represents a plurality of VGAM24 host target genes. The mRNA of each one of this plurality of VGAM24 host target genes comprises one or more host target binding sites, each having a nucleotide sequence which is at least partly complementary to VGAM24 RNA, herein designated VGAM RNA, and which when bound by VGAM24 RNA causes inhibition of translation of respective one or more VGAM24 host target proteins.

It is further appreciated by one skilled in the art that the mode of translational inhibition illustrated by FIG. 1 with specific reference to translational inhibition exerted by VGAM24 gene, herein designated VGAM GENE, on one or more VGAM24 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM24 is inhibition of expression of host target genes, as part of a novel viral mechanism of attacking a host. Accordingly, utilities of VGAM24 include diagnosis, prevention and treatment of viral infection by Vaccinia Virus. Specific functions, and accordingly utilities, of VGAM24 correlate with, and may be deduced from, the identity of the host target genes which VGAM24 binds and inhibits, and the function of these host target genes, as elaborated hereinbelow.

Nucleotide sequences of the VGAM24 precursor RNA, herein designated VGAM PRECURSOR RNA, and of the ‘diced’ VGAM24 RNA, herein designated VGAM RNA, and a schematic representation of the secondary folding of VGAM24 folded precursor RNA, herein designated VGAM FOLDED PRECURSOR RNA, of VGAM24 are further described hereinbelow with reference to Table 1.

Nucleotide sequences of host target binding sites, such as BINDING SITE-I, BINDING SITE-II and BINDING SITE-III of FIG. 1, found on VGAM24 host target RNA, and schematic representation of the complementarity of each of these host target binding sites to VGAM24 RNA, herein designated VGAM RNA, are described hereinbelow with reference to Table 2.

As mentioned hereinabove with reference to FIG. 1, a function of VGAM24 gene, herein designated VGAM is inhibition of expression of VGAM24 target genes. It is appreciated that specific functions, and accordingly utilities, of VGAM24 correlate with, and may be deduced from, the identity of the target genes which VGAM24 binds and inhibits, and the function of these target genes, as elaborated hereinbelow.

Calpain 2, (m/II) Large Subunit (CAPN2, Accession NM_{—}001748) is a VGAM24 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM24 RNA, herein designated VGAM RNA, also designated SEQ ID:359.

A function of VGAM24 is therefore inhibition of Calpain 2, (m/II) Large Subunit (CAPN2, Accession NM_{—}001748). Accordingly, utilities of VGAM24 include diagnosis, prevention and treatment of diseases and clinical conditions associated with CAPN2. Dishevelled Associated Activator of Morphogenesis 2 (DAAM2, Accession XM_{—}166434) is another VGAM24 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM24 RNA, herein designated VGAM RNA, also designated SEQ ID:359.

Another function of VGAM24 is therefore inhibition of Dishevelled Associated Activator of Morphogenesis 2 (DAAM2, Accession XM_{—}166434), a gene which controls cell polarity and movement during development. Accordingly, utilities of VGAM24 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM24 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM24 RNA, herein designated VGAM RNA, also designated SEQ ID:359.

Another function of VGAM24 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM24 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM24 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM24 RNA, herein designated VGAM RNA, also designated SEQ ID:359.

Another function of VGAM24 is therefore inhibition of KIAA0475 (Accession NM_{—}014864). Accordingly, utilities of VGAM24 include diagnosis, prevention and treatment of diseases and clinical conditions associated with KIAA0475. LOC222671 (Accession XM_{—}167094) is another VGAM24 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM24 RNA, herein designated VGAM RNA, also designated SEQ ID:359.

Another function of VGAM24 is therefore inhibition of LOC222671 (Accession XM_{—}167094). Accordingly, utilities of VGAM24 include diagnosis, prevention and treatment of diseases and clinical conditions associated with LOC222671. LOC254901 (Accession XM_{—}171878) is another VGAM24 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM24 RNA, herein designated VGAM RNA, also designated SEQ ID:359.

Another function of VGAM24 is therefore inhibition of LOC254901 (Accession XM_{—}171878). Accordingly, utilities of VGAM24 include diagnosis, prevention and treatment of diseases and clinical conditions associated with LOC254901. LOC257615 (Accession XM_{—}175293) is another VGAM24 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM24 RNA, herein designated VGAM RNA, also designated SEQ ID:359.

Another function of VGAM24 is therefore inhibition of LOC257615 (Accession XM_{—}175293). Accordingly, utilities of VGAM24 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 25 (VGAM25) viral gene, which modulates expression of respective host target genes thereof, the function and utility of which host target genes is known in the art.

VGAM25 is a novel bioinformatically detected regulatory, non protein coding, viral micro RNA (miRNA) gene. The method by which VGAM25 was detected is described hereinabove with reference to FIGS. 1-8.

VGAM25 gene, herein designated VGAM GENE, is a viral gene contained in the genome of Vaccinia Virus. VGAM25 host target gene, herein designated VGAM HOST TARGET GENE, is a human gene contained in the human genome.

VGAM25 gene encodes a VGAM25 precursor RNA, herein designated VGAM PRECURSOR RNA. Similar to other miRNA genes, and unlike most ordinary genes, VGAM25 precursor RNA does not encode a protein. A nucleotide sequence identical or highly similar to the nucleotide sequence of VGAM25 precursor RNA is designated SEQ ID:11, and is provided hereinbelow with reference to the sequence listing part. Nucleotide sequence SEQ ID:11 is located at position 188927 relative to the genome of Vaccinia Virus.

VGAM25 precursor RNA folds onto itself, forming VGAM25 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM25 folded precursor RNA into VGAM25 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM25 RNA is designated SEQ ID:360, and is provided hereinbelow with reference to the sequence listing part.

VGAM25 host target gene, herein designated VGAM HOST TARGET GENE, encodes a corresponding messenger RNA, VGAM25 host target RNA, herein designated VGAM HOST TARGET RNA. VGAM25 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM25 RNA, herein designated VGAM RNA, binds complementarily to one or more host target binding sites located in untranslated regions of VGAM25 host target RNA, herein designated VGAM HOST TARGET RNA. This complementary binding is due to the fact that the nucleotide sequence of VGAM25 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM25 RNA, herein designated VGAM RNA, may have a different number of host target binding sites in untranslated regions of a VGAM25 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM25 RNA, herein designated VGAM RNA, to host target binding sites on VGAM25 host target RNA, herein designated VGAM HOST TARGET RNA, such as BINDING SITE I, BINDING SITE II and BINDING SITE III, inhibits translation of VGAM25 host target RNA into VGAM25 host target protein, herein designated VGAM HOST TARGET PROTEIN. VGAM host target protein is therefore outlined by a broken line.

It is appreciated that VGAM25 host target gene, herein designated VGAM HOST TARGET GENE, in fact represents a plurality of VGAM25 host target genes. The mRNA of each one of this plurality of VGAM25 host target genes comprises one or more host target binding sites, each having a nucleotide sequence which is at least partly complementary to VGAM25 RNA, herein designated VGAM RNA, and which when bound by VGAM25 RNA causes inhibition of translation of respective one or more VGAM25 host target proteins.

It is further appreciated by one skilled in the art that the mode of translational inhibition illustrated by FIG. 1 with specific reference to translational inhibition exerted by VGAM25 gene, herein designated VGAM GENE, on one or more VGAM25 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM25 is inhibition of expression of host target genes, as part of a novel viral mechanism of attacking a host. Accordingly, utilities of VGAM25 include diagnosis, prevention and treatment of viral infection by Vaccinia Virus. Specific functions, and accordingly utilities, of VGAM25 correlate with, and may be deduced from, the identity of the host target genes which VGAM25 binds and inhibits, and the function of these host target genes, as elaborated hereinbelow.

Nucleotide sequences of the VGAM25 precursor RNA, herein designated VGAM PRECURSOR RNA, and of the ‘diced’ VGAM25 RNA, herein designated VGAM RNA, and a schematic representation of the secondary folding of VGAM25 folded precursor RNA, herein designated VGAM FOLDED PRECURSOR RNA, of VGAM25 are further described hereinbelow with reference to Table 1.

Nucleotide sequences of host target binding sites, such as BINDING SITE-I, BINDING SITE-II and BINDING SITE-III of FIG. 1, found on VGAM25 host target RNA, and schematic representation of the complementarity of each of these host target binding sites to VGAM25 RNA, herein designated VGAM RNA, are described hereinbelow with reference to Table 2.

As mentioned hereinabove with reference to FIG. 1, a function of VGAM25 gene, herein designated VGAM is inhibition of expression of VGAM25 target genes. It is appreciated that specific functions, and accordingly utilities, of VGAM25 correlate with, and may be deduced from, the identity of the target genes which VGAM25 binds and inhibits, and the function of these target genes, as elaborated hereinbelow.

Calpain 2, (m/II) Large Subunit (CAPN2, Accession NM_{—}001748) is a VGAM25 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM25 RNA, herein designated VGAM RNA, also designated SEQ ID:360.

A function of VGAM25 is therefore inhibition of Calpain 2, (m/II) Large Subunit (CAPN2, Accession NM_{—}001748). Accordingly, utilities of VGAM25 include diagnosis, prevention and treatment of diseases and clinical conditions associated with CAPN2. Dishevelled Associated Activator of Morphogenesis 2 (DAAM2, Accession XM_{—}166434) is another VGAM25 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM25 RNA, herein designated VGAM RNA, also designated SEQ ID:360.

Another function of VGAM25 is therefore inhibition of Dishevelled Associated Activator of Morphogenesis 2 (DAAM2, Accession XM_{—}166434), a gene which controls cell polarity and movement during development. Accordingly, utilities of VGAM25 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM25 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM25 RNA, herein designated VGAM RNA, also designated SEQ ID:360.

Another function of VGAM25 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM25 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM25 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM25 RNA, herein designated VGAM RNA, also designated SEQ ID:360.

Another function of VGAM25 is therefore inhibition of KIAA0475 (Accession NM_{—}014864). Accordingly, utilities of VGAM25 include diagnosis, prevention and treatment of diseases and clinical conditions associated with KIAA0475. LOC222671 (Accession XM_{—}167094) is another VGAM25 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM25 RNA, herein designated VGAM RNA, also designated SEQ ID:360.

Another function of VGAM25 is therefore inhibition of LOC222671 (Accession XM_{—}167094). Accordingly, utilities of VGAM25 include diagnosis, prevention and treatment of diseases and clinical conditions associated with LOC222671. LOC254901 (Accession XM_{—}171878) is another VGAM25 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM25 RNA, herein designated VGAM RNA, also designated SEQ ID:360.

Another function of VGAM25 is therefore inhibition of LOC254901 (Accession XM_{—}171878). Accordingly, utilities of VGAM25 include diagnosis, prevention and treatment of diseases and clinical conditions associated with LOC254901. LOC257615 (Accession XM_{—}175293) is another VGAM25 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM25 RNA, herein designated VGAM RNA, also designated SEQ ID:360.

Another function of VGAM25 is therefore inhibition of LOC257615 (Accession XM_{—}175293). Accordingly, utilities of VGAM25 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 26 (VGAM26) viral gene, which modulates expression of respective host target genes thereof, the function and utility of which host target genes is known in the art.

VGAM26 is a novel bioinformatically detected regulatory, non protein coding, viral micro RNA (miRNA) gene. The method by which VGAM26 was detected is described hereinabove with reference to FIGS. 1-8.

VGAM26 gene, herein designated VGAM GENE, is a viral gene contained in the genome of Vaccinia Virus. VGAM26 host target gene, herein designated VGAM HOST TARGET GENE, is a human gene contained in the human genome.

VGAM26 gene encodes a VGAM26 precursor RNA, herein designated VGAM PRECURSOR RNA. Similar to other miRNA genes, and unlike most ordinary genes, VGAM26 precursor RNA does not encode a protein. A nucleotide sequence identical or highly similar to the nucleotide sequence of VGAM26 precursor RNA is designated SEQ ID:12, and is provided hereinbelow with reference to the sequence listing part. Nucleotide sequence SEQ ID:12 is located at position 189067 relative to the genome of Vaccinia Virus.

VGAM26 precursor RNA folds onto itself, forming VGAM26 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM26 folded precursor RNA into VGAM26 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM26 RNA is designated SEQ ID:361, and is provided hereinbelow with reference to the sequence listing part.

VGAM26 host target gene, herein designated VGAM HOST TARGET GENE, encodes a corresponding messenger RNA, VGAM26 host target RNA, herein designated VGAM HOST TARGET RNA. VGAM26 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM26 RNA, herein designated VGAM RNA, binds complementarily to one or more host target binding sites located in untranslated regions of VGAM26 host target RNA, herein designated VGAM HOST TARGET RNA. This complementary binding is due to the fact that the nucleotide sequence of VGAM26 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM26 RNA, herein designated VGAM RNA, may have a different number of host target binding sites in untranslated regions of a VGAM26 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM26 RNA, herein designated VGAM RNA, to host target binding sites on VGAM26 host target RNA, herein designated VGAM HOST TARGET RNA, such as BINDING SITE I, BINDING SITE II and BINDING SITE III, inhibits translation of VGAM26 host target RNA into VGAM26 host target protein, herein designated VGAM HOST TARGET PROTEIN. VGAM host target protein is therefore outlined by a broken line.

It is appreciated that VGAM26 host target gene, herein designated VGAM HOST TARGET GENE, in fact represents a plurality of VGAM26 host target genes. The mRNA of each one of this plurality of VGAM26 host target genes comprises one or more host target binding sites, each having a nucleotide sequence which is at least partly complementary to VGAM26 RNA, herein designated VGAM RNA, and which when bound by VGAM26 RNA causes inhibition of translation of respective one or more VGAM26 host target proteins.

It is further appreciated by one skilled in the art that the mode of translational inhibition illustrated by FIG. 1 with specific reference to translational inhibition exerted by VGAM26 gene, herein designated VGAM GENE, on one or more VGAM26 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM26 is inhibition of expression of host target genes, as part of a novel viral mechanism of attacking a host. Accordingly, utilities of VGAM26 include diagnosis, prevention and treatment of viral infection by Vaccinia Virus. Specific functions, and accordingly utilities, of VGAM26 correlate with, and may be deduced from, the identity of the host target genes which VGAM26 binds and inhibits, and the function of these host target genes, as elaborated hereinbelow.

Nucleotide sequences of the VGAM26 precursor RNA, herein designated VGAM PRECURSOR RNA, and of the ‘diced’ VGAM26 RNA, herein designated VGAM RNA, and a schematic representation of the secondary folding of VGAM26 folded precursor RNA, herein designated VGAM FOLDED PRECURSOR RNA, of VGAM26 are further described hereinbelow with reference to Table 1.

Nucleotide sequences of host target binding sites, such as BINDING SITE-I, BINDING SITE-II and BINDING SITE-III of FIG. 1, found on VGAM26 host target RNA, and schematic representation of the complementarity of each of these host target binding sites to VGAM26 RNA, herein designated VGAM RNA, are described hereinbelow with reference to Table 2.

As mentioned hereinabove with reference to FIG. 1, a function of VGAM26 gene, herein designated VGAM is inhibition of expression of VGAM26 target genes. It is appreciated that specific functions, and accordingly utilities, of VGAM26 correlate with, and may be deduced from, the identity of the target genes which VGAM26 binds and inhibits, and the function of these target genes, as elaborated hereinbelow.

Integrin, Beta 7 (ITGB7, Accession NM_{—}000889) is a VGAM26 host target gene. ITGB7 BINDING SITE is HOST TARGET binding site found in the 5′ untranslated region of mRNA encoded by ITGB7, corresponding to a HOST TARGET binding site such as BINDING SITE I, BINDING SITE II or BINDING SITE III. Table 2 illustrates the complementarity of the nucleotide sequences of ITGB7 BINDING SITE, designated SEQ ID:783, to the nucleotide sequence of VGAM26 RNA, herein designated VGAM RNA, also designated SEQ ID:361.

A function of VGAM26 is therefore inhibition of Integrin, Beta 7 (ITGB7, Accession NM_{—}000889), a gene which may play a role in adhesive interactions of leukocytes. Accordingly, utilities of VGAM26 include diagnosis, prevention and treatment of diseases and clinical conditions associated with ITGB7. The function of ITGB7 has been established by previous studies. The integrins are a large family of heterodimeric cell-surface glycoproteins that play key roles in the adherence of cells to other cells and to extracellular matrix proteins. Leukocytes express a variety of integrins that are crucial participants in the inflammatory and immune responses. All integrins are composed of 1 alpha subunit and 1 beta subunit that are translated separately and are noncovalently associated. The cDNA sequences of 6 mammalian beta subunits, numbered beta-1 through beta-6, had previously been reported. Erie et al. (1991) determined the complete sequence of a novel integrin beta subunit from leukocytes, designated beta-7. The cDNA contained a single large open reading frame predicted to encode a 798-amino acid protein precursor (signal peptide plus mature protein). Like other beta-subunit proteins, it was predicted to contain a large extracellular domain, a transmembrane domain, and a cytoplasmic tail. The deduced beta-7 amino acid sequence was 32 to 46% identical to the 6 previously sequenced subunits. It was most similar to the leukocyte integrin common beta subunit (ITGB2; CD18; 116920). Erle et al. (1991) predicted that integrin beta-7 plays a role in adhesive interactions of leukocytes. Krissansen et al. (1992) assigned the ITGB7 gene to human chromosome 12 by analysis of somatic cell hybrids. Krissansen et al. (1992) commented on the fact that the gene encoding the alpha subunit of the fibronectin receptor (FNRA; 135620) resides on chromosome 12 also. They stated that the ‘extent and evolutionary significance of linkages between integrin genes . . . remains to be determined.’ Baker et al. (1992) assigned the ITGB7 gene to 12q13.13 by nonisotopic in situ hybridization. Yuan et al. (1992) mapped the mouse homolog Itgb-7 to chromosome 15 by study of mouse-hamster somatic cell hybrids and by interspecific backcrosses.

Full details of the abovementioned studies are described in the following publications, the disclosure of which are hereby incorporated by reference:
Baker, E.; Sutherland, G. R.; Jiang, W.-M.; Yuan, Q.; Leung, E.; Watson, J. D.; Krissansen, G. W.: Mapping of the human integrin beta-7 gene (ITG-beta-7) to 12q13.13 by non-isotopic in situ hybridization. Mammalian Genome 2: 272-273, 1992.; and
Erle, D. J.; Ruegg, C.; Sheppard, D.; Pytela, R.: Complete amino acid sequence of an integrin beta subunit (beta-7) identified in leukocytes. J. Biol. Chem. 266: 11009-11016, 1991.

Further studies establishing the function and utilities of ITGB7 are found in John Hopkins OMIM database record ID 147559, and in sited publications numbered 3017-3018, 301 and 3019 listed in the bibliography section hereinbelow, which are also hereby incorporated by reference. RPP30 (Accession NM_{—}006413) is another VGAM26 host target gene. RPP30 BINDING SITE is HOST TARGET binding site found in the 3′ untranslated region of mRNA encoded by RPP30, corresponding to a HOST TARGET binding site such as BINDING SITE I, BINDING SITE II or BINDING SITE III. Table 2 illustrates the complementarity of the nucleotide sequences of RPP30 BINDING SITE, designated SEQ ID:1297, to the nucleotide sequence of VGAM26 RNA, herein designated VGAM RNA, also designated SEQ ID:361.

Another function of VGAM26 is therefore inhibition of RPP30 (Accession NM_{—}006413), a gene which is a component of ribonuclease p that processes 5′ ends of precursor tRNAs. Accordingly, utilities of VGAM26 include diagnosis, prevention and treatment of diseases and clinical conditions associated with RPP30. The function of RPP30 has been established by previous studies. By biochemical purification of RNase P, micropeptide sequence analysis, and EST database searching, Eder et al. (1997) obtained a cDNA encoding RPP30. The deduced protein contains 268 amino acids with a predicted molecular mass of nearly 30 kD. Jarrous et al. (1998) determined that RPP30 is a target for antisera from systemic sclerosis patients. Immunoprecipitation analysis showed that polyclonal antibodies raised against RPP20, RPP30, RPP38, or RPP40 interact with RNase P from HeLa cells.

Full details of the abovementioned studies are described in the following publications, the disclosure of which are hereby incorporated by reference:
Eder, P. S.; Kekuda, R.; Stolc, V.; Altman, S.: Characterization of two scleroderma autoimmune antigens that copurify with human ribonuclease P. Proc. Nat. Acad. Sci. 94: 1101-1106, 1997.; and
Jarrous, N.; Eder, P. S.; Guerrier-Takada, C.; Hoog, C.; Altman, S.: Autoantigenic properties of some protein subunits of catalytically active complexes of human ribonuclease P. RNA 4: 407.

Further studies establishing the function and utilities of RPP30 are found in John Hopkins OMIM database record ID 606115, and in sited publications numbered 18 and 209 listed in the bibliography section hereinbelow, which are also hereby incorporated by reference. FLJ13263 (Accession NM_{—}025125) is another VGAM26 host target gene. FLJ13263 BINDING SITE is HOST TARGET binding site found in the 3′ untranslated region of mRNA encoded by FLJ13263, corresponding to a HOST TARGET binding site such as BINDING SITE I, BINDING SITE II or BINDING SITE III. Table 2 illustrates the complementarity of the nucleotide sequences of FLJ13263 BINDING SITE, designated SEQ ID:2139, to the nucleotide sequence of VGAM26 RNA, herein designated VGAM RNA, also designated SEQ ID:361.

Another function of VGAM26 is therefore inhibition of FLJ13263 (Accession NM_{—}025125). Accordingly, utilities of VGAM26 include diagnosis, prevention and treatment of diseases and clinical conditions associated with FLJ13263. FLJ23074 (Accession NM_{—}025052) is another VGAM26 host target gene. FLJ23074 BINDING SITE is HOST TARGET binding site found in the 5′ untranslated region of mRNA encoded by FLJ23074, corresponding to a HOST TARGET binding site such as BINDING SITE I, BINDING SITE II or BINDING SITE III. Table 2 illustrates the complementarity of the nucleotide sequences of FLJ23074 BINDING SITE, designated SEQ ID:2133, to the nucleotide sequence of VGAM26 RNA, herein designated VGAM RNA, also designated SEQ ID:361.

Another function of VGAM26 is therefore inhibition of FLJ23074 (Accession NM_{—}025052). Accordingly, utilities of VGAM26 include diagnosis, prevention and treatment of diseases and clinical conditions associated with FLJ23074. General Transcription Factor IIA, 2, 12 kDa (GTF2A2, Accession NM_{—}004492) is another VGAM26 host target gene. GTF2A2 BINDING SITE is HOST TARGET binding site found in the 3′ untranslated region of mRNA encoded by GTF2A2, corresponding to a HOST TARGET binding site such as BINDING SITE I, BINDING SITE II or BINDING SITE III. Table 2 illustrates the complementarity of the nucleotide sequences of GTF2A2 BINDING SITE, designated SEQ ID:1119, to the nucleotide sequence of VGAM26 RNA, herein designated VGAM RNA, also designated SEQ ID:361.

Another function of VGAM26 is therefore inhibition of General Transcription Factor IIA, 2, 12 kDa (GTF2A2, Accession NM_{—}004492). Accordingly, utilities of VGAM26 include diagnosis, prevention and treatment of diseases and clinical conditions associated with GTF2A2. IMP-2 (Accession NM_{—}006548) is another VGAM26 host target gene. IMP-2 BINDING SITE is HOST TARGET binding site found in the 3′ untranslated region of mRNA encoded by IMP-2, corresponding to a HOST TARGET binding site such as BINDING SITE I, BINDING SITE II or BINDING SITE III. Table 2 illustrates the complementarity of the nucleotide sequences of IMP-2 BINDING SITE, designated SEQ ID:1308, to the nucleotide sequence of VGAM26 RNA, herein designated VGAM RNA, also designated SEQ ID:361.

Another function of VGAM26 is therefore inhibition of IMP-2 (Accession NM_{—}006548). Accordingly, utilities of VGAM26 include diagnosis, prevention and treatment of diseases and clinical conditions associated with IMP-2. KIAA0212 (Accession NM_{—}014674) is another VGAM26 host target gene. KIAA0212 BINDING SITE is HOST TARGET binding site found in the 3′ untranslated region of mRNA encoded by KIAA0212, corresponding to a HOST TARGET binding site such as BINDING SITE I, BINDING SITE II or BINDING SITE III. Table 2 illustrates the complementarity of the nucleotide sequences of KIAA0212 BINDING SITE, designated SEQ ID:1518, to the nucleotide sequence of VGAM26 RNA, herein designated VGAM RNA, also designated SEQ ID:361.

Another function of VGAM26 is therefore inhibition of KIAA0212 (Accession NM_{—}014674). Accordingly, utilities of VGAM26 include diagnosis, prevention and treatment of diseases and clinical conditions associated with KIAA0212. Oxysterol Binding Protein-like 2 (OSBPL2, Accession NM_{—}014835) is another VGAM26 host target gene. OSBPL2 BINDING SITE1 and OSBPL2 BINDING SITE2 are HOST TARGET binding sites found in untranslated regions of mRNA encoded by OSBPL2, corresponding to HOST TARGET binding sites such as BINDING SITE I, BINDING SITE II or BINDING SITE III. Table 2 illustrates the complementarity of the nucleotide sequences of OSBPL2 BINDING SITE1 and OSBPL2 BINDING SITE2, designated SEQ ID:1566 and SEQ ID:2482 respectively, to the nucleotide sequence of VGAM26 RNA, herein designated VGAM RNA, also designated SEQ ID:361.

Another function of VGAM26 is therefore inhibition of Oxysterol Binding Protein-like 2 (OSBPL2, Accession NM_{—}014835). Accordingly, utilities of VGAM26 include diagnosis, prevention and treatment of diseases and clinical conditions associated with OSBPL2. LOC254826 (Accession XM_{—}173188) is another VGAM26 host target gene. LOC254826 BINDING SITE is HOST TARGET binding site found in the 3′ untranslated region of mRNA encoded by LOC254826, corresponding to a HOST TARGET binding site such as BINDING SITE I, BINDING SITE II or BINDING SITE III. Table 2 illustrates the complementarity of the nucleotide sequences of LOC254826 BINDING SITE, designated SEQ ID:3732, to the nucleotide sequence of VGAM26 RNA, herein designated VGAM RNA, also designated SEQ ID:361.

Another function of VGAM26 is therefore inhibition of LOC254826 (Accession XM_{—}173188). Accordingly, utilities of VGAM26 include diagnosis, prevention and treatment of diseases and clinical conditions associated with LOC254826. FIG. 1 further provides a conceptual description of a novel bioinformatically detected viral gene of the present invention, referred to here as Viral Genomic Address Messenger 27 (VGAM27) viral gene, which modulates expression of respective host target genes thereof, the function and utility of which host target genes is known in the art.

VGAM27 is a novel bioinformatically detected regulatory, non protein coding, viral micro RNA (miRNA) gene. The method by which VGAM27 was detected is described hereinabove with reference to FIGS. 1-8.

VGAM27 gene, herein designated VGAM GENE, is a viral gene contained in the genome of Vaccinia Virus. VGAM27 host target gene, herein designated VGAM HOST TARGET GENE, is a human gene contained in the human genome.

VGAM27 gene encodes a VGAM27 precursor RNA, herein designated VGAM PRECURSOR RNA. Similar to other miRNA genes, and unlike most ordinary genes, VGAM27 precursor RNA does not encode a protein. A nucleotide sequence identical or highly similar to the nucleotide sequence of VGAM27 precursor RNA is designated SEQ ID:13, and is provided hereinbelow with reference to the sequence listing part. Nucleotide sequence SEQ ID:13 is located at position 190758 relative to the genome of Vaccinia Virus.

VGAM27 precursor RNA folds onto itself, forming VGAM27 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM27 folded precursor RNA into VGAM27 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM27 RNA is designated SEQ ID:362, and is provided hereinbelow with reference to the sequence listing part.

VGAM27 host target gene, herein designated VGAM HOST TARGET GENE, encodes a corresponding messenger RNA, VGAM27 host target RNA, herein designated VGAM HOST TARGET RNA. VGAM27 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM27 RNA, herein designated VGAM RNA, binds complementarily to one or more host target binding sites located in untranslated regions of VGAM27 host target RNA, herein designated VGAM HOST TARGET RNA. This complementary binding is due to the fact that the nucleotide sequence of VGAM27 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM27 RNA, herein designated VGAM RNA, may have a different number of host target binding sites in untranslated regions of a VGAM27 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM27 RNA, herein designated VGAM RNA, to host target binding sites on VGAM27 host target RNA, herein designated VGAM HOST TARGET RNA, such as BINDING SITE I, BINDING SITE II and BINDING SITE III, inhibits translation of VGAM27 host target RNA into VGAM27 host target protein, herein designated VGAM HOST TARGET PROTEIN. VGAM host target protein is therefore outlined by a broken line.

It is appreciated that VGAM27 host target gene, herein designated VGAM HOST TARGET GENE, in fact represents a plurality of VGAM27 host target genes. The mRNA of each one of this plurality of VGAM27 host target genes comprises one or more host target binding sites, each having a nucleotide sequence which is at least partly complementary to VGAM27 RNA, herein designated VGAM RNA, and which when bound by VGAM27 RNA causes inhibition of translation of respective one or more VGAM27 host target proteins.

It is further appreciated by one skilled in the art that the mode of translational inhibition illustrated by FIG. 1 with specific reference to translational inhibition exerted by VGAM27 gene, herein designated VGAM GENE, on one or more VGAM27 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM27 is inhibition of expression of host target genes, as part of a novel viral mechanism of attacking a host. Accordingly, utilities of VGAM27 include diagnosis, prevention and treatment of viral infection by Vaccinia Virus. Specific functions, and accordingly utilities, of VGAM27 correlate with, and may be deduced from, the identity of the host target genes which VGAM27 binds and inhibits, and the function of these host target genes, as elaborated hereinbelow.

Nucleotide sequences of the VGAM27 precursor RNA, herein designated VGAM PRECURSOR RNA, and of the ‘diced’ VGAM27 RNA, herein designated VGAM RNA, and a schematic representation of the secondary folding of VGAM27 folded precursor RNA, herein designated VGAM FOLDED PRECURSOR RNA, of VGAM27 are further described hereinbelow with reference to Table 1.

Nucleotide sequences of host target binding sites, such as BINDING SITE-I, BINDING SITE-II and BINDING SITE-III of FIG. 1, found on VGAM27 host target RNA, and schematic representation of the complementarity of each of these host target binding sites to VGAM27 RNA, herein designated VGAM RNA, are described hereinbelow with reference to Table 2.

As mentioned hereinabove with reference to FIG. 1, a function of VGAM27 gene, herein designated VGAM is inhibition of expression of VGAM27 target genes. It is appreciated that specific functions, and accordingly utilities, of VGAM27 correlate with, and may be deduced from, the identity of the target genes which VGAM27 binds and inhibits, and the function of these target genes, as elaborated hereinbelow.

Calpain 2, (m/II) Large Subunit (CAPN2, Accession NM_{—}001748) is a VGAM27 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM27 RNA, herein designated VGAM RNA, also designated SEQ ID:362.

A function of VGAM27 is therefore inhibition of Calpain 2, (m/II) Large Subunit (CAPN2, Accession NM_{—}001748). Accordingly, utilities of VGAM27 include diagnosis, prevention and treatment of diseases and clinical conditions associated with CAPN2. Dishevelled Associated Activator of Morphogenesis 2 (DAAM2, Accession XM_{—}166434) is another VGAM27 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM27 RNA, herein designated VGAM RNA, also designated SEQ ID:362.

Another function of VGAM27 is therefore inhibition of Dishevelled Associated Activator of Morphogenesis 2 (DAAM2, Accession XM_{—}166434), a gene which controls cell polarity and movement during development. Accordingly, utilities of VGAM27 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM27 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM27 RNA, herein designated VGAM RNA, also designated SEQ ID:362.

Another function of VGAM27 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM27 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM27 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM27 RNA, herein designated VGAM RNA, also designated SEQ ID:362.

Another function of VGAM27 is therefore inhibition of KIAA0475 (Accession NM_{—}014864). Accordingly, utilities of VGAM27 include diagnosis, prevention and treatment of diseases and clinical conditions associated with KIAA0475. LOC222671 (Accession XM_{—}167094) is another VGAM27 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM27 RNA, herein designated VGAM RNA, also designated SEQ ID:362.

Another function of VGAM27 is therefore inhibition of LOC222671 (Accession XM_{—}167094). Accordingly, utilities of VGAM27 include diagnosis, prevention and treatment of diseases and clinical conditions associated with LOC222671. LOC254901 (Accession XM_{—}171878) is another VGAM27 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM27 RNA, herein designated VGAM RNA, also designated SEQ ID:362.

Another function of VGAM27 is therefore inhibition of LOC254901 (Accession XM_{—}171878). Accordingly, utilities of VGAM27 include diagnosis, prevention and treatment of diseases and clinical conditions associated with LOC254901. LOC257615 (Accession XM_{—}175293) is another VGAM27 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM27 RNA, herein designated VGAM RNA, also designated SEQ ID:362.

Another function of VGAM27 is therefore inhibition of LOC257615 (Accession XM_{—}175293). Accordingly, utilities of VGAM27 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 28 (VGAM28) viral gene, which modulates expression of respective host target genes thereof, the function and utility of which host target genes is known in the art.

VGAM28 is a novel bioinformatically detected regulatory, non protein coding, viral micro RNA (miRNA) gene. The method by which VGAM28 was detected is described hereinabove with reference to FIGS. 1-8.

VGAM28 gene, herein designated VGAM GENE, is a viral gene contained in the genome of Vaccinia Virus. VGAM28 host target gene, herein designated VGAM HOST TARGET GENE, is a human gene contained in the human genome.

VGAM28 gene encodes a VGAM28 precursor RNA, herein designated VGAM PRECURSOR RNA. Similar to other miRNA genes, and unlike most ordinary genes, VGAM28 precursor RNA does not encode a protein. A nucleotide sequence identical or highly similar to the nucleotide sequence of VGAM28 precursor RNA is designated SEQ ID:14, and is provided hereinbelow with reference to the sequence listing part. Nucleotide sequence SEQ ID: 14 is located at position 190758 relative to the genome of Vaccinia Virus.

VGAM28 precursor RNA folds onto itself, forming VGAM28 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM28 folded precursor RNA into VGAM28 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM28 RNA is designated SEQ ID:363, and is provided hereinbelow with reference to the sequence listing part.

VGAM28 host target gene, herein designated VGAM HOST TARGET GENE, encodes a corresponding messenger RNA, VGAM28 host target RNA, herein designated VGAM HOST TARGET RNA. VGAM28 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM28 RNA, herein designated VGAM RNA, binds complementarily to one or more host target binding sites located in untranslated regions of VGAM28 host target RNA, herein designated VGAM HOST TARGET RNA. This complementary binding is due to the fact that the nucleotide sequence of VGAM28 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM28 RNA, herein designated VGAM RNA, may have a different number of host target binding sites in untranslated regions of a VGAM28 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM28 RNA, herein designated VGAM RNA, to host target binding sites on VGAM28 host target RNA, herein designated VGAM HOST TARGET RNA, such as BINDING SITE I, BINDING SITE II and BINDING SITE III, inhibits translation of VGAM28 host target RNA into VGAM28 host target protein, herein designated VGAM HOST TARGET PROTEIN. VGAM host target protein is therefore outlined by a broken line.

It is appreciated that VGAM28 host target gene, herein designated VGAM HOST TARGET GENE, in fact represents a plurality of VGAM28 host target genes. The mRNA of each one of this plurality of VGAM28 host target genes comprises one or more host target binding sites, each having a nucleotide sequence which is at least partly complementary to VGAM28 RNA, herein designated VGAM RNA, and which when bound by VGAM28 RNA causes inhibition of translation of respective one or more VGAM28 host target proteins.

It is further appreciated by one skilled in the art that the mode of translational inhibition illustrated by FIG. 1 with specific reference to translational inhibition exerted by VGAM28 gene, herein designated VGAM GENE, on one or more VGAM28 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM28 is inhibition of expression of host target genes, as part of a novel viral mechanism of attacking a host. Accordingly, utilities of VGAM28 include diagnosis, prevention and treatment of viral infection by Vaccinia Virus. Specific functions, and accordingly utilities, of VGAM28 correlate with, and may be deduced from, the identity of the host target genes which VGAM28 binds and inhibits, and the function of these host target genes, as elaborated hereinbelow.

Nucleotide sequences of the VGAM28 precursor RNA, herein designated VGAM PRECURSOR RNA, and of the ‘diced’ VGAM28 RNA, herein designated VGAM RNA, and a schematic representation of the secondary folding of VGAM28 folded precursor RNA, herein designated VGAM FOLDED PRECURSOR RNA, of VGAM28 are further described hereinbelow with reference to Table 1.

Nucleotide sequences of host target binding sites, such as BINDING SITE-I, BINDING SITE-II and BINDING SITE-III of FIG. 1, found on VGAM28 host target RNA, and schematic representation of the complementarity of each of these host target binding sites to VGAM28 RNA, herein designated VGAM RNA, are described hereinbelow with reference to Table 2.

As mentioned hereinabove with reference to FIG. 1, a function of VGAM28 gene, herein designated VGAM is inhibition of expression of VGAM28 target genes. It is appreciated that specific functions, and accordingly utilities, of VGAM28 correlate with, and may be deduced from, the identity of the target genes which VGAM28 binds and inhibits, and the function of these target genes, as elaborated hereinbelow.

Calpain 2, (m/II) Large Subunit (CAPN2, Accession NM_{—}001748) is a VGAM28 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM28 RNA, herein designated VGAM RNA, also designated SEQ ID:363.

A function of VGAM28 is therefore inhibition of Calpain 2, (m/II) Large Subunit (CAPN2, Accession NM_{—}001748). Accordingly, utilities of VGAM28 include diagnosis, prevention and treatment of diseases and clinical conditions associated with CAPN2. Dishevelled Associated Activator of Morphogenesis 2 (DAAM2, Accession XM_{—}166434) is another VGAM28 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM28 RNA, herein designated VGAM RNA, also designated SEQ ID:363.

Another function of VGAM28 is therefore inhibition of Dishevelled Associated Activator of Morphogenesis 2 (DAAM2, Accession XM_{—}166434), a gene which controls cell polarity and movement during development. Accordingly, utilities of VGAM28 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM28 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM28 RNA, herein designated VGAM RNA, also designated SEQ ID:363.

Another function of VGAM28 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM28 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM28 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM28 RNA, herein designated VGAM RNA, also designated SEQ ID:363.

Another function of VGAM28 is therefore inhibition of KIAA0475 (Accession NM_{—}014864). Accordingly, utilities of VGAM28 include diagnosis, prevention and treatment of diseases and clinical conditions associated with KIAA0475. LOC222671 (Accession XM_{—}167094) is another VGAM28 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM28 RNA, herein designated VGAM RNA, also designated SEQ ID:363.

Another function of VGAM28 is therefore inhibition of LOC222671 (Accession XM_{—}167094). Accordingly, utilities of VGAM28 include diagnosis, prevention and treatment of diseases and clinical conditions associated with LOC222671. LOC254901 (Accession XM_{—}171878) is another VGAM28 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM28 RNA, herein designated VGAM RNA, also designated SEQ ID:363.

Another function of VGAM28 is therefore inhibition of LOC254901 (Accession XM_{—}171878). Accordingly, utilities of VGAM28 include diagnosis, prevention and treatment of diseases and clinical conditions associated with LOC254901. LOC257615 (Accession XM_{—}175293) is another VGAM28 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM28 RNA, herein designated VGAM RNA, also designated SEQ ID:363.

Another function of VGAM28 is therefore inhibition of LOC257615 (Accession XM_{—}175293). Accordingly, utilities of VGAM28 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 29 (VGAM29) viral gene, which modulates expression of respective host target genes thereof, the function and utility of which host target genes is known in the art.

VGAM29 is a novel bioinformatically detected regulatory, non protein coding, viral micro RNA (miRNA) gene. The method by which VGAM29 was detected is described hereinabove with reference to FIGS. 1-8.

VGAM29 gene, herein designated VGAM GENE, is a viral gene contained in the genome of Vaccinia Virus. VGAM29 host target gene, herein designated VGAM HOST TARGET GENE, is a human gene contained in the human genome.

VGAM29 gene encodes a VGAM29 precursor RNA, herein designated VGAM PRECURSOR RNA. Similar to other miRNA genes, and unlike most ordinary genes, VGAM29 precursor RNA does not encode a protein. A nucleotide sequence identical or highly similar to the nucleotide sequence of VGAM29 precursor RNA is designated SEQ ID:15, and is provided hereinbelow with reference to the sequence listing part. Nucleotide sequence SEQ ID:15 is located at position 188937 relative to the genome of Vaccinia Virus.

VGAM29 precursor RNA folds onto itself, forming VGAM29 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM29 folded precursor RNA into VGAM29 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM29 RNA is designated SEQ ID:364, and is provided hereinbelow with reference to the sequence listing part.

VGAM29 host target gene, herein designated VGAM HOST TARGET GENE, encodes a corresponding messenger RNA, VGAM29 host target RNA, herein designated VGAM HOST TARGET RNA. VGAM29 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM29 RNA, herein designated VGAM RNA, binds complementarily to one or more host target binding sites located in untranslated regions of VGAM29 host target RNA, herein designated VGAM HOST TARGET RNA. This complementary binding is due to the fact that the nucleotide sequence of VGAM29 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM29 RNA, herein designated VGAM RNA, may have a different number of host target binding sites in untranslated regions of a VGAM29 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM29 RNA, herein designated VGAM RNA, to host target binding sites on VGAM29 host target RNA, herein designated VGAM HOST TARGET RNA, such as BINDING SITE I, BINDING SITE II and BINDING SITE III, inhibits translation of VGAM29 host target RNA into VGAM29 host target protein, herein designated VGAM HOST TARGET PROTEIN. VGAM host target protein is therefore outlined by a broken line.

It is appreciated that VGAM29 host target gene, herein designated VGAM HOST TARGET GENE, in fact represents a plurality of VGAM29 host target genes. The mRNA of each one of this plurality of VGAM29 host target genes comprises one or more host target binding sites, each having a nucleotide sequence which is at least partly complementary to VGAM29 RNA, herein designated VGAM RNA, and which when bound by VGAM29 RNA causes inhibition of translation of respective one or more VGAM29 host target proteins.

It is further appreciated by one skilled in the art that the mode of translational inhibition illustrated by FIG. 1 with specific reference to translational inhibition exerted by VGAM29 gene, herein designated VGAM GENE, on one or more VGAM29 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM29 is inhibition of expression of host target genes, as part of a novel viral mechanism of attacking a host. Accordingly, utilities of VGAM29 include diagnosis, prevention and treatment of viral infection by Vaccinia Virus. Specific functions, and accordingly utilities, of VGAM29 correlate with, and may be deduced from, the identity of the host target genes which VGAM29 binds and inhibits, and the function of these host target genes, as elaborated hereinbelow.

Nucleotide sequences of the VGAM29 precursor RNA, herein designated VGAM PRECURSOR RNA, and of the ‘diced’ VGAM29 RNA, herein designated VGAM RNA, and a schematic representation of the secondary folding of VGAM29 folded precursor RNA, herein designated VGAM FOLDED PRECURSOR RNA, of VGAM29 are further described hereinbelow with reference to Table 1.

Nucleotide sequences of host target binding sites, such as BINDING SITE-I, BINDING SITE-II and BINDING SITE-III of FIG. 1, found on VGAM29 host target RNA, and schematic representation of the complementarity of each of these host target binding sites to VGAM29 RNA, herein designated VGAM RNA, are described hereinbelow with reference to Table 2.

As mentioned hereinabove with reference to FIG. 1, a function of VGAM29 gene, herein designated VGAM is inhibition of expression of VGAM29 target genes. It is appreciated that specific functions, and accordingly utilities, of VGAM29 correlate with, and may be deduced from, the identity of the target genes which VGAM29 binds and inhibits, and the function of these target genes, as elaborated hereinbelow.

Calpain 2, (m/II) Large Subunit (CAPN2, Accession NM_{—}001748) is a VGAM29 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM29 RNA, herein designated VGAM RNA, also designated SEQ ID:364.

A function of VGAM29 is therefore inhibition of Calpain 2, (m/II) Large Subunit (CAPN2, Accession NM_{—}001748). Accordingly, utilities of VGAM29 include diagnosis, prevention and treatment of diseases and clinical conditions associated with CAPN2. Dishevelled Associated Activator of Morphogenesis 2 (DAAM2, Accession XM_{—}166434) is another VGAM29 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM29 RNA, herein designated VGAM RNA, also designated SEQ ID:364.

Another function of VGAM29 is therefore inhibition of Dishevelled Associated Activator of Morphogenesis 2 (DAAM2, Accession XM_{—}166434), a gene which controls cell polarity and movement during development. Accordingly, utilities of VGAM29 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM29 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM29 RNA, herein designated VGAM RNA, also designated SEQ ID:364.

Another function of VGAM29 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM29 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM29 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1570, to the nucleotide sequence of VGAM29 RNA, herein designated VGAM RNA, also designated SEQ ID:364.

Another function of VGAM29 is therefore inhibition of KIAA0475 (Accession NM_{—}014864). Accordingly, utilities of VGAM29 include diagnosis, prevention and treatment of diseases and clinical conditions associated with KIAA0475. LOC222671 (Accession XM_{—}167094) is another VGAM29 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM29 RNA, herein designated VGAM RNA, also designated SEQ ID:364.

Another function of VGAM29 is therefore inhibition of LOC222671 (Accession XM_{—}167094). Accordingly, utilities of VGAM29 include diagnosis, prevention and treatment of diseases and clinical conditions associated with LOC222671. LOC254901 (Accession XM_{—}171878) is another VGAM29 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM29 RNA, herein designated VGAM RNA, also designated SEQ ID:364.

Another function of VGAM29 is therefore inhibition of LOC254901 (Accession XM_{—}171878). Accordingly, utilities of VGAM29 include diagnosis, prevention and treatment of diseases and clinical conditions associated with LOC254901. LOC257615 (Accession XM_{—}175293) is another VGAM29 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM29 RNA, herein designated VGAM RNA, also designated SEQ ID:364.

Another function of VGAM29 is therefore inhibition of LOC257615 (Accession XM_{—}175293). Accordingly, utilities of VGAM29 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 30 (VGAM30) viral gene, which modulates expression of respective host target genes thereof, the function and utility of which host target genes is known in the art.

VGAM30 is a novel bioinformatically detected regulatory, non protein coding, viral micro RNA (miRNA) gene. The method by which VGAM30 was detected is described hereinabove with reference to FIGS. 1-8.

VGAM30 gene, herein designated VGAM GENE, is a viral gene contained in the genome of Vaccinia Virus. VGAM30 host target gene, herein designated VGAM HOST TARGET GENE, is a human gene contained in the human genome.

VGAM30 gene encodes a VGAM30 precursor RNA, herein designated VGAM PRECURSOR RNA. Similar to other miRNA genes, and unlike most ordinary genes, VGAM30 precursor RNA does not encode a protein. A nucleotide sequence identical or highly similar to the nucleotide sequence of VGAM30 precursor RNA is designated SEQ ID:16, and is provided hereinbelow with reference to the sequence listing part. Nucleotide sequence SEQ ID: 16 is located at position 187719 relative to the genome of Vaccinia Virus.

VGAM30 precursor RNA folds onto itself, forming VGAM30 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM30 folded precursor RNA into VGAM30 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM30 RNA is designated SEQ ID:365, and is provided hereinbelow with reference to the sequence listing part.

VGAM30 host target gene, herein designated VGAM HOST TARGET GENE, encodes a corresponding messenger RNA, VGAM30 host target RNA, herein designated VGAM HOST TARGET RNA. VGAM30 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM30 RNA, herein designated VGAM RNA, binds complementarily to one or more host target binding sites located in untranslated regions of VGAM30 host target RNA, herein designated VGAM HOST TARGET RNA. This complementary binding is due to the fact that the nucleotide sequence of VGAM30 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM30 RNA, herein designated VGAM RNA, may have a different number of host target binding sites in untranslated regions of a VGAM30 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM30 RNA, herein designated VGAM RNA, to host target binding sites on VGAM30 host target RNA, herein designated VGAM HOST TARGET RNA, such as BINDING SITE I, BINDING SITE II and BINDING SITE III, inhibits translation of VGAM30 host target RNA into VGAM30 host target protein, herein designated VGAM HOST TARGET PROTEIN. VGAM host target protein is therefore outlined by a broken line.

It is appreciated that VGAM30 host target gene, herein designated VGAM HOST TARGET GENE, in fact represents a plurality of VGAM30 host target genes. The mRNA of each one of this plurality of VGAM30 host target genes comprises one or more host target binding sites, each having a nucleotide sequence which is at least partly complementary to VGAM30 RNA, herein designated VGAM RNA, and which when bound by VGAM30 RNA causes inhibition of translation of respective one or more VGAM30 host target proteins.

It is further appreciated by one skilled in the art that the mode of translational inhibition illustrated by FIG. 1 with specific reference to translational inhibition exerted by VGAM30 gene, herein designated VGAM GENE, on one or more VGAM30 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM30 is inhibition of expression of host target genes, as part of a novel viral mechanism of attacking a host. Accordingly, utilities of VGAM30 include diagnosis, prevention and treatment of viral infection by Vaccinia Virus. Specific functions, and accordingly utilities, of VGAM30 correlate with, and may be deduced from, the identity of the host target genes which VGAM30 binds and inhibits, and the function of these host target genes, as elaborated hereinbelow.

Nucleotide sequences of the VGAM30 precursor RNA, herein designated VGAM PRECURSOR RNA, and of the ‘diced’ VGAM30 RNA, herein designated VGAM RNA, and a schematic representation of the secondary folding of VGAM30 folded precursor RNA, herein designated VGAM FOLDED PRECURSOR RNA, of VGAM30 are further described hereinbelow with reference to Table 1.

Nucleotide sequences of host target binding sites, such as BINDING SITE-I, BINDING SITE-II and BINDING SITE-III of FIG. 1, found on VGAM30 host target RNA, and schematic representation of the complementarity of each of these host target binding sites to VGAM30 RNA, herein designated VGAM RNA, are described hereinbelow with reference to Table 2.

As mentioned hereinabove with reference to FIG. 1, a function of VGAM30 gene, herein designated VGAM is inhibition of expression of VGAM30 target genes. It is appreciated that specific functions, and accordingly utilities, of VGAM30 correlate with, and may be deduced from, the identity of the target genes which VGAM30 binds and inhibits, and the function of these target genes, as elaborated hereinbelow.

Fibroblast Growth Factor 2 (basic) (FGF2, Accession NM_{—}002006) is a VGAM30 host target gene. FGF2 BINDING SITE is HOST TARGET binding site found in the 3′ untranslated region of mRNA encoded by FGF2, corresponding to a HOST TARGET binding site such as BINDING SITE I, BINDING SITE II or BINDING SITE III. Table 2 illustrates the complementarity of the nucleotide sequences of FGF2 BINDING SITE, designated SEQ ID:884, to the nucleotide sequence of VGAM30 RNA, herein designated VGAM RNA, also designated SEQ ID:365.

A function of VGAM30 is therefore inhibition of Fibroblast Growth Factor 2 (basic) (FGF2, Accession NM_{—}002006), a gene which Basic fibroblast growth factor 2. Accordingly, utilities of VGAM30 include diagnosis, prevention and treatment of diseases and clinical conditions associated with FGF2. The function of FGF2 has been established by previous studies. FGF2 is a wide-spectrum mitogenic, angiogenic, and neurotrophic factor that is expressed at low levels in many tissues and cell types and reaches high concentrations in brain and pituitary. FGF2 has been implicated in a multitude of physiologic and pathologic processes, including limb development, angiogenesis, wound healing, and tumor growth Abraham et al. (1986) isolated a clone encoding FGFB from a bovine pituitary cDNA library. Southern blot analysis and genomic cloning of the human gene indicated that basic FGF is encoded by a single gene split by at least 2 introns of size greater than 15 kb (Abraham et al., 1986). Kurokawa et al. (1987) isolated a cDNA for basic FGF. The 4-kb cDNA had a coding sequence, 5-prime and 3-prime untranslated regions, and a poly(A) chain. Mergia et al. (1986) used a bovine basic FGF cDNA as a hybridization probe in Southern blot analysis of DNAs isolated from a panel of mouse-human cell hybrids.

They concluded that FGFB is on chromosome 4, which carries other growth factors: EGF (OMIM Ref. No. 131530) and TCGF (OMIM Ref. No. 147680). Lafage-Pochitaloff et al. (1990) mapped the FGFB gene to 4q26-q27 by in situ hybridization. By in situ hybridization to human metaphase and prometaphase chromosomes, Fukushima et al. (1990) concluded that the FGFB gene maps to 4q25. Using in situ chromosomal hybridization, Mattei et al. (1992) demonstrated that the corresponding gene in the mouse is on chromosome 3. Animal model experiments lend further support to the function of FGF2. To investigate the role of FGF2 in bone, Montero et al. (2000) examined mice with a disruption of the Fgf2 gene. They found a significant decrease in trabecular bone volume, mineral apposition, and bone formation rates. In addition, there was a profound decreased mineralization of bone marrow stromal cultures from Fgf2-deficient mice. The results showed that FGF2 helps determine bone mass as well as bone formation.

It is appreciated that the abovementioned animal model for FGF2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Abraham, J. A.; Mergia, A.; Whang, J. L.; Tumolo, A.; Friedman, J.; Hjerild, K. A.; Gospodarowicz, D.; Fiddes, J. C.: Nucleotide sequence of a bovine clone encoding the angiogenic protein, basic fibroblast growth factor. Science 233: 545-548, 1986.; and
Montero, A.; Okada, Y.; Tomita, M.; Ito, M.; Tsurukami, H.; Nakamura, T.; Doetschman, T.; Coffin, J. D.; Hurley, M. M.: Disruption of the fibroblast growth factor-2 gene results in dec.

Further studies establishing the function and utilities of FGF2 are found in John Hopkins OMIM database record ID 134920, and in sited publications numbered 32-35, 2730-37, 609, 2731-41, 2732-4 and 2733 listed in the bibliography section hereinbelow, which are also hereby incorporated by reference. M-phase Phosphoprotein 1 (MPHOSPH1, Accession NM_{—}016195) is another VGAM30 host target gene. MPHOSPH1 BINDING SITE is HOST TARGET binding site found in the 3′ untranslated region of mRNA encoded by MPHOSPH1, corresponding to a HOST TARGET binding site such as BINDING SITE I, BINDING SITE II or BINDING SITE III. Table 2 illustrates the complementarity of the nucleotide sequences of MPHOSPH1 BINDING SITE, designated SEQ ID: 1670, to the nucleotide sequence of VGAM30 RNA, herein designated VGAM RNA, also designated SEQ ID:365.

Another function of VGAM30 is therefore inhibition of M-phase Phosphoprotein 1 (MPHOSPH1, Accession NM_{—}016195), a gene which is Phosphorylated during M-phase and interacts with guanosine triphosphate. Accordingly, utilities of VGAM30 include diagnosis, prevention and treatment of diseases and clinical conditions associated with MPHOSPH1. The function of MPHOSPH1 has been established by previous studies. By treating bacterial expression libraries with M-phase cytosol containing the relevant kinases, followed by immunoscreening with MPM2 antibody, Westendorf et al. (1994) isolated partial cDNAs encoding MPHOSPH1, which they termed MPP1, and MPHOSPH2, which they termed MPP2 (FOXM1; 602341). Sequence analysis predicted that the partial MPHOSPH1 cDNA encodes a 566-amino acid protein, the first 302 amino acids of which form a coiled-coil alpha helix. The authors determined that the MPM2 antibody-reactive sites of the MPHOSPH proteins consist of 5-amino acid stretches containing a hydrophobic residue, the putative phosphorylated residue (proline), another hydrophobic residue, and finally a basic or hydrophobic residue (e.g., LTPLK).

Full details of the abovementioned studies are described in the following publications, the disclosure of which are hereby incorporated by reference:
Matsumoto-Taniura, N.; Pirollet, F.; Monroe, R.; Gerace, L.; Westendorf, J. M.: Identification of novel M phase phosphoproteins by expression cloning. Molec. Biol. Cell 7: 1455-1469, 1996.; and
Westendorf, J. M.; Rao, P. N.; Gerace, L.: Cloning of cDNAs for M-phase phosphoproteins recognized by the MPM2 monoclonal antibody and determination of the phosphorylated epitope. Proc. Na.

Further studies establishing the function and utilities of MPHOSPH1 are found in John Hopkins OMIM database record ID 605498, and in sited publications numbered 165 and 1459 listed in the bibliography section hereinbelow, which are also hereby incorporated by reference. FLJ11274 (Accession NM_{—}018375) is another VGAM30 host target gene. FLJ11274 BINDING SITE is HOST TARGET binding site found in the 3′ untranslated region of mRNA encoded by FLJ11274, corresponding to a HOST TARGET binding site such as BINDING SITE I, BINDING SITE II or BINDING SITE III. Table 2 illustrates the complementarity of the nucleotide sequences of FLJ11274 BINDING SITE, designated SEQ ID:1820, to the nucleotide sequence of VGAM30 RNA, herein designated VGAM RNA, also designated SEQ ID:365.

Another function of VGAM30 is therefore inhibition of FLJ11274 (Accession NM_{—}018375). Accordingly, utilities of VGAM30 include diagnosis, prevention and treatment of diseases and clinical conditions associated with FLJ11274. FLJ31101 (Accession NM_{—}017964) is another VGAM30 host target gene. FLJ31101 BINDING SITE is HOST TARGET binding site found in the 3′ untranslated region of mRNA encoded by FLJ31101, corresponding to a HOST TARGET binding site such as BINDING SITE I, BINDING SITE II or BINDING SITE III. Table 2 illustrates the complementarity of the nucleotide sequences of FLJ31101 BINDING SITE, designated SEQ ID:1771, to the nucleotide sequence of VGAM30 RNA, herein designated VGAM RNA, also designated SEQ ID:365.

Another function of VGAM30 is therefore inhibition of FLJ31101 (Accession NM_{—}017964). Accordingly, utilities of VGAM30 include diagnosis, prevention and treatment of diseases and clinical conditions associated with FLJ31101. Glutamic Pyruvate Transaminase (alanine aminotransferase) 2 (GPT2, Accession NM_{—}133443) is another VGAM30 host target gene. GPT2 BINDING SITE is HOST TARGET binding site found in the 3′ untranslated region of mRNA encoded by GPT2, corresponding to a HOST TARGET binding site such as BINDING SITE I, BINDING SITE II or BINDING SITE III. Table 2 illustrates the complementarity of the nucleotide sequences of GPT2 BINDING SITE, designated SEQ ID:2417, to the nucleotide sequence of VGAM30 RNA, herein designated VGAM RNA, also designated SEQ ID:365.

Another function of VGAM30 is therefore inhibition of Glutamic Pyruvate Transaminase (alanine aminotransferase) 2 (GPT2, Accession NM_{—}133443). Accordingly, utilities of VGAM30 include diagnosis, prevention and treatment of diseases and clinical conditions associated with GPT2. RMP (Accession NM_{—}134447) is another VGAM30 host target gene. RMP BINDING SITE1 and RMP BINDING SITE2 are HOST TARGET binding sites found in untranslated regions of mRNA encoded by RMP, corresponding to HOST TARGET binding sites such as BINDING SITE I, BINDING SITE II or BINDING SITE III. Table 2 illustrates the complementarity of the nucleotide sequences of RMP BINDING SITE1 and RMP BINDING SITE2, designated SEQ ID:2430 and SEQ ID: 1059 respectively, to the nucleotide sequence of VGAM30 RNA, herein designated VGAM RNA, also designated SEQ ID:365.

Another function of VGAM30 is therefore inhibition of RMP (Accession NM_{—}134447). Accordingly, utilities of VGAM30 include diagnosis, prevention and treatment of diseases and clinical conditions associated with RMP. LOC136288 (Accession XM_{—}059832) is another VGAM30 host target gene. LOC136288 BINDING SITE is HOST TARGET binding site found in the 3′ untranslated region of mRNA encoded by LOC136288, corresponding to a HOST TARGET binding site such as BINDING SITE I, BINDING SITE II or BINDING SITE III. Table 2 illustrates the complementarity of the nucleotide sequences of LOC136288 BINDING SITE, designated SEQ ID:3018, to the nucleotide sequence of VGAM30 RNA, herein designated VGAM RNA, also designated SEQ ID:365.

Another function of VGAM30 is therefore inhibition of LOC136288 (Accession XM_{—}059832). Accordingly, utilities of VGAM30 include diagnosis, prevention and treatment of diseases and clinical conditions associated with LOC136288. FIG. 1 further provides a conceptual description of a novel bioinformatically detected viral gene of the present invention, referred to here as Viral Genomic Address Messenger 31 (VGAM31) viral gene, which modulates expression of respective host target genes thereof, the function and utility of which host target genes is known in the art.

VGAM31 is a novel bioinformatically detected regulatory, non protein coding, viral micro RNA (miRNA) gene. The method by which VGAM31 was detected is described hereinabove with reference to FIGS. 1-8.

VGAM31 gene, herein designated VGAM GENE, is a viral gene contained in the genome of Vaccinia Virus. VGAM31 host target gene, herein designated VGAM HOST TARGET GENE, is a human gene contained in the human genome.

VGAM31 gene encodes a VGAM31 precursor RNA, herein designated VGAM PRECURSOR RNA. Similar to other miRNA genes, and unlike most ordinary genes, VGAM31 precursor RNA does not encode a protein. A nucleotide sequence identical or highly similar to the nucleotide sequence of VGAM31 precursor RNA is designated SEQ ID:17, and is provided hereinbelow with reference to the sequence listing part. Nucleotide sequence SEQ ID:17 is located at position 188420 relative to the genome of Vaccinia Virus.

VGAM31 precursor RNA folds onto itself, forming VGAM31 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM31 folded precursor RNA into VGAM31 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM31 RNA is designated SEQ ID:366, and is provided hereinbelow with reference to the sequence listing part.

VGAM31 host target gene, herein designated VGAM HOST TARGET GENE, encodes a corresponding messenger RNA, VGAM31 host target RNA, herein designated VGAM HOST TARGET RNA. VGAM31 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM31 RNA, herein designated VGAM RNA, binds complementarily to one or more host target binding sites located in untranslated regions of VGAM31 host target RNA, herein designated VGAM HOST TARGET RNA. This complementary binding is due to the fact that the nucleotide sequence of VGAM31 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM31 RNA, herein designated VGAM RNA, may have a different number of host target binding sites in untranslated regions of a VGAM31 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM31 RNA, herein designated VGAM RNA, to host target binding sites on VGAM31 host target RNA, herein designated VGAM HOST TARGET RNA, such as BINDING SITE I, BINDING SITE II and BINDING SITE III, inhibits translation of VGAM31 host target RNA into VGAM31 host target protein, herein designated VGAM HOST TARGET PROTEIN. VGAM host target protein is therefore outlined by a broken line.

It is appreciated that VGAM31 host target gene, herein designated VGAM HOST TARGET GENE, in fact represents a plurality of VGAM31 host target genes. The mRNA of each one of this plurality of VGAM31 host target genes comprises one or more host target binding sites, each having a nucleotide sequence which is at least partly complementary to VGAM31 RNA, herein designated VGAM RNA, and which when bound by VGAM31 RNA causes inhibition of translation of respective one or more VGAM31 host target proteins.

It is further appreciated by one skilled in the art that the mode of translational inhibition illustrated by FIG. 1 with specific reference to translational inhibition exerted by VGAM31 gene, herein designated VGAM GENE, on one or more VGAM31 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM31 is inhibition of expression of host target genes, as part of a novel viral mechanism of attacking a host. Accordingly, utilities of VGAM31 include diagnosis, prevention and treatment of viral infection by Vaccinia Virus. Specific functions, and accordingly utilities, of VGAM31 correlate with, and may be deduced from, the identity of the host target genes which VGAM31 binds and inhibits, and the function of these host target genes, as elaborated hereinbelow.

Nucleotide sequences of the VGAM31 precursor RNA, herein designated VGAM PRECURSOR RNA, and of the ‘diced’ VGAM31 RNA, herein designated VGAM RNA, and a schematic representation of the secondary folding of VGAM31 folded precursor RNA, herein designated VGAM FOLDED PRECURSOR RNA, of VGAM31 are further described hereinbelow with reference to Table 1.

Nucleotide sequences of host target binding sites, such as BINDING SITE-I, BINDING SITE-II and BINDING SITE-III of FIG. 1, found on VGAM31 host target RNA, and schematic representation of the complementarity of each of these host target binding sites to VGAM31 RNA, herein designated VGAM RNA, are described hereinbelow with reference to Table 2.

As mentioned hereinabove with reference to FIG. 1, a function of VGAM31 gene, herein designated VGAM is inhibition of expression of VGAM31 target genes. It is appreciated that specific functions, and accordingly utilities, of VGAM31 correlate with, and may be deduced from, the identity of the target genes which VGAM31 binds and inhibits, and the function of these target genes, as elaborated hereinbelow.

Activation-induced Cytidine Deaminase (AICDA, Accession NM_{—}020661) is a VGAM31 host target gene. AICDA BINDING SITE is HOST TARGET binding site found in the 3′ untranslated region of mRNA encoded by AICDA, corresponding to a HOST TARGET binding site such as BINDING SITE I, BINDING SITE II or BINDING SITE III. Table 2 illustrates the complementarity of the nucleotide sequences of AICDA BINDING SITE, designated SEQ ID:1922, to the nucleotide sequence of VGAM31 RNA, herein designated VGAM RNA, also designated SEQ ID:366.

A function of VGAM31 is therefore inhibition of Activation-induced Cytidine Deaminase (AICDA, Accession NM_{—}020661), a gene which a member of the cytidine deaminase family. Accordingly, utilities of VGAM31 include diagnosis, prevention and treatment of diseases and clinical conditions associated with AICDA. The function of AICDA has been established by previous studies. Muramatsu et al. (1999) isolated the gene encoding activation-induced cytidine deaminase (Aid), a member of the cytidine deaminase family, from a murine B-cell lymphoma line induced by combined stimulation of transforming growth factor-beta (TGFB; 190180), interleukin-4 (IL4; 147780), and CD40 ligand (CD40L; 300386). Muto et al. (2000) isolated the human AID gene, which encodes a 198-amino acid protein containing a conserved cytidine deaminase motif. The human AID protein shares 92% amino acid identity with mouse Aid. RT-PCR analysis of 15 human tissues detected strong expression of AID mRNA in lymph nodes and tonsils. Petersen-Mahrt et al. (2002) hypothesized that the 3 gene diversification processes mediated by AICDA, somatic hypermutation, gene conversion, and class-switch recombination, may be initiated by DNA lesions at dC/dG pairs by AICDA, which has sequence homology to the RNA editing enzyme APOBEC1 (OMIM Ref. No. 600130). Expression of AICDA in E. coli confers a mutator phenotype yielding nucleotide transitions at dC/dG in a context-dependent manner. The mutation triggered by AICDA is enhanced by a deficiency of uracil-DNA glycosylase (UNG; 191525), indicating that AICDA functions by deaminating dC residues in DNA. Petersen-Mahrt et al. (2002) proposed that diversification of functional Ig genes is triggered by AICDA-mediated deamination of dC residues in the Ig locus with the outcome, i.e., hypermutation phases 1 and 2, gene conversion, or switch recombination, dependent on the way in which the initiating dU/dG lesion is resolved. Muto et al. (2000) mapped the AID gene to 12p13 by FISH. Animal model experiments lend further support to the function of AICDA. Muramatsu et al. (2000) found that in the mouse, Aid deficiency caused a complete defect in class switching and showed a hyper-IgM phenotype with enlarged germinal centers containing strongly activated B cells before or after immunization. Mouse Aid −/− spleen cells stimulated in vitro with lipopolysaccharide (LPS) and cytokines failed to undergo CSR, although they expressed germline transcripts.

It is appreciated that the abovementioned animal model for AICDA is acknowledged by those skilled in the art as a scientifically valid animal model, as can be further appreciated from the publications sited hereinbelow.

Full details of the abovementioned studies are described in the following publications, the disclosure of which are hereby incorporated by reference:
Muramatsu, M.; Sankaranand, V. S.; Anant, S.; Sugai, M.; Kinoshita, K.; Davidson, N. O.; Honjo, T.: Specific expression of activation-induced cytidine deaminase (AID), a novel member of the RNA-editing deaminase family in germinal center B cells. J. Biol. Chem. 274: 18470-18476, 1999.; and
Muramatsu, M.; Kinoshita, K.; Fagarasan, S.; Yamada, S.; Shinkai, Y.; Honjo, T.: Class switch recombination and hypermutation require activation-induced cytidine deaminase (AID), a pot.

Further studies establishing the function and utilities of AICDA are found in John Hopkins OMIM database record ID 605257, and in sited publications numbered 984-987, 106 and 2054-531 listed in the bibliography section hereinbelow, which are also hereby incorporated by reference. AS3 (Accession NM_{—}015928) is another VGAM31 host target gene. AS3 BINDING SITE is HOST TARGET binding site found in the 3′ untranslated region of mRNA encoded by AS3, corresponding to a HOST TARGET binding site such as BINDING SITE I, BINDING SITE II or BINDING SITE III. Table 2 illustrates the complementarity of the nucleotide sequences of AS3 BINDING SITE, designated SEQ ID: 1652, to the nucleotide sequence of VGAM31 RNA, herein designated VGAM RNA, also designated SEQ ID:366.

Another function of VGAM31 is therefore inhibition of AS3 (Accession NM_{—}015928), a gene which inhibits cell proloferation. Accordingly, utilities of VGAM31 include diagnosis, prevention and treatment of diseases and clinical conditions associated with AS3. The function of AS3 has been established by previous studies. In the prostate of adult mammals, most epithelial cells are in a state of proliferative quiescence. Androgens regulate this effect by inducing cell cycle arrest in the G0/G1 phase. Geck et al. (2000) identified potential mediators of this androgen-induced proliferative shutoff by means of subtractive cDNA libraries. The expression pattern of one of these sequences, designated AS3, strongly correlated with the expression of the androgen-induced proliferative shutoff both temporally and dosewise. The AS3 gene is up-regulated during androgen-induced proliferative shutoff and induces cell proliferation arrest when expressed in a retrovirus transduced model. The deduced 1,391-amino acid AS3 protein has putative transactivating features, protein-protein interaction motifs (coiled coil and leucine zipper), and DNA-binding domains, suggesting that AS3 is a transcription factor. AS3 also has a protein-kinase motif, suggesting that it may act by phosphorylating a target protein. Geck et al. (1999) demonstrated that the transcript of the AS3 gene has 34 exons spanning approximately 200 kb of genomic DNA. By homology searching in GenBank, they demonstrated that the AS3 gene lies on 13q12-q13, downstream of the breast cancer susceptibility gene BRCA2 (OMIM Ref. No. 600185) and centromeric to the retinoblastoma (RB1; 180200) locus. Geck et al. (2001) presented data on the location of the AS3 gene in relation to BRCA2 and pointed out that the D13S171 marker, which had been widely used as an intragenic marker of BRCA2, is actually located in the center of the 200-kb AS3 gene. The microsatellite instability of the S171 marker links the AS3 gene to a variety of cancers.

Full details of the abovementioned studies are described in the following publications, the disclosure of which are hereby incorporated by reference:
Geck, P.; Sonnenschein, C.; Soto, A. M.: The D13S171 marker, misannotated to BRCA2, links the AS3 gene to various cancers. (Letter) Am. J. Hum. Genet. 69: 461-463, 2001.; and
Geck, P.; Szelei, J.; Jimenez, J.; Sonnenschein, C.; Soto, A. M.: Early gene expression during androgen-induced inhibition of proliferation of prostate cancer cells: a new suppressor.

Further studies establishing the function and utilities of AS3 are found in John Hopkins OMIM database record ID 605333, and in sited publications numbered 1070-527 listed in the bibliography section hereinbelow, which are also hereby incorporated by reference. Epilepsy, Progressive Myoclonus Type 2, Lafora Disease (laforin) (EPM2A, Accession NM_{—}005670) is another VGAM31 host target gene. EPM2A BINDING SITE is HOST TARGET binding site found in the 3′ untranslated region of mRNA encoded by EPM2A, corresponding to a HOST TARGET binding site such as BINDING SITE I, BINDING SITE II or BINDING SITE III. Table 2 illustrates the complementarity of the nucleotide sequences of EPM2A BINDING SITE, designated SEQ ID:1234, to the nucleotide sequence of VGAM31 RNA, herein designated VGAM RNA, also designated SEQ ID:366.

Another function of VGAM31 is therefore inhibition of Epilepsy, Progressive Myoclonus Type 2, Lafora Disease (laforin) (EPM2A, Accession NM_{—}005670), a gene which Laforin; protein tyrosine phosphatase that may have role in glycogen metabolism. Accordingly, utilities of VGAM31 include diagnosis, prevention and treatment of diseases and clinical conditions associated with EPM2A. The function of EPM2A has been established by previous studies. In the Lafora type, onset takes the form of grand mal seizures and/or myoclonus at about age 15 years. Rapid and severe mental deterioration ensues, often with psychotic features. Survival is short, less than 10 years after onset. Histologic study of the brain shows Lafora bodies (which may also be demonstrable on muscle and liver biopsy). Intracellular Lafora bodies suggesting amyloid are found in the brain, and similar inclusions in the cells of the heart and liver (Harriman and Millar, 1955). The Lafora material has the properties of an acid mucopolysaccharide. Yokoi et al. (1968) arrived at a preliminary conclusion that the Lafora body is polyglucosan in nature. They pictured the existence of an enzyme defect which leads to deposition of polyglucosans near their site of synthesis in the agranular endoplasmic reticulum. Schwarz and Yanoff (1965) described a brother and sister, offspring of a one-and-one-half cousin marriage, with this disease. Seizures began at age 15 in the boy with slowly progressive motor and mental deterioration leading to death at age 23.5 years. The sister's seizures began at age 14 years and progression to dementia and blindness occurred, with death at age 19. Intra- and extracellular Lafora bodies were found in the CNS, retina, axis cylinders of spinal nerves, heart muscle, liver cells, and striated muscle fibers. Diagnosis by liver or muscle biopsy was proposed. Busard et al. (1986, 1987) demonstrated that the diagnosis can be made reliably on axillary skin biopsy; all patients show typical periodic acid-Schiff (PAS)-positive inclusions in the myoepithelial cells of the secretory acini of the apocrine glands and/or in the cells of the eccrine duct. The method has no value for carrier detection. In cultured fibroblasts, Fluharty et al. (1970) described bodies which may be the equivalent of the Lafora body observed histologically. Sarlin et al. (1960) claimed that electroencephalographic abnormalities distinguished heterozygotes from homozygous normals. Norio and Koskiniemi (1979), as well as others, have concluded that there are 3 types of what they termed progressive myoclonic epilepsy (PME). The Lafora type shows onset of grand mal seizures and/or myoclonus around the fifteenth year of life; rapid and severe mental deterioration, often with psychotic symptoms; short survival; histologic finding of Lafora bodies; and autosomal recessive inheritance. The Unverricht-Lundborg type (EPM1; 254800), which is frequent in Finland, has onset around the tenth year; variable severity; progressive incapacitation from myoclonus associated with mild mental symptoms; variable survival; ‘degenerative’ histologic changes; and autosomal recessive inheritance. A dominant form, to which Hartung's name is attached (see OMIM Ref. No. 159600), has been described. By linkage studies in 3 Italian families with Lafora disease, Lehesjoki et al. (1992) demonstrated that the gene is located at a locus other than that for the Unverricht-Lundborg type on chromosome 21q22.3. Serratosa et al. (1995) studied linkage in 9 families in which Lafora disease had been proven by biopsy in at least 1 member. Using microsatellite markers spaced an average of 13 cM apart, they used linkage analysis in all 9 families and homozygosity mapping in 4 consanguineous families to assign the gene for Lafora disease to 6q23-q25. An extended pedigree with 5 affected members independently proved linkage. The multipoint 1-Iod unit support interval covered a 2.5-cM region surrounding D6S403. Homozygosity mapping defined a 17-cM region in 6q23-q25 flanked by D6S292 and D6S420. The 9 families with a total of 19 patients affected with Lafora disease originated from the United States, Spain, Palestine, and Iran. Maddox et al. (1997) studied a 2-generation family in which a recombination event reduced the Lafora critical region to a 4-cM interval flanked by markers D6S308 and D6S311. Sainz et al. (1997) narrowed the assignment of the MELF locus within 6q24 by study of recombinants and homozygosities. Ganesh et al. (2000) cloned and expressed the full-length 38-kD laforin protein in transfected cells. Recombinant laforin was able to hydrolyze phosphotyrosine as well as phosphoserine/threonine substrates, demonstrating that laforin is an active dual-specificity phosphatase. Biochemical, immunofluorescence, and ultrastructural studies on transfected HeLa cells revealed that laforin is a cytoplasmic protein associated with polyribosomes. Expression of 2 proteins with missense mutations seen in EPM2A patients resulted in ubiquitin-positive perinuclear aggregates, suggesting that these were misfolded proteins targeted for degradation. The authors suggested that laforin is involved in translational regulation and that protein misfolding may be one of the molecular bases of the Lafora disease phenotype caused by missense mutations in the EPM2A gene. Gomez-Garre et al. (2000) reported the complete coding sequence of the EPM2A gene, including the ATG initiation codon region. They used SSCP analysis of the 4 exons in 34 unrelated patients with Lafora disease and identified EPM2A mutations in 27 (79%) of them (49 of 68 chromosomes, or 72%). The patients originated from Spain, Italy, Australia, Holland, the US, North Africa, Turkey, and France. A total of 20 different EPM2A mutations, 11 of them novel, were characterized. The authors summarized 25 EPM2A mutations distributed throughout the gene in 44 unrelated Lafora disease patients. The mutations included 10 deletions of different sizes, 9 missense mutations, 3 nonsense mutations, and 3 frameshift mutations. The R241X mutation (254780.0008) was encountered in almost 40% of the probands. In 5 Lafora disease families (13% of the families studied), Gomez-Garre et al. (2000) excluded linkage to the EPM2A gene region.

Full details of the abovementioned studies are described in the following publications, the disclosure of which are hereby incorporated by reference:
Norio, R.; Koskiniemi, M.: Progressive myoclonus epilepsy: genetic and nosological aspects with special reference to 107 Finnish patients. Clin. Genet. 15: 382-398, 1979.; and
Gomez-Garre, P.; Sanz, Y.; Rodriguez de Cordoba, S.; Serratosa, J. M.: Mutational spectrum of the EPM2A gene in progressive myoclonus epilepsy of Lafora: high degree of allelic heterogen.

Further studies establishing the function and utilities of EPM2A are found in John Hopkins OMIM database record ID 254780, and in sited publications numbered 2041-2049, 2069-207 and 2078-2077 listed in the bibliography section hereinbelow, which are also hereby incorporated by reference. Histamine Receptor H1 (HRH1, Accession NM_{—}000861) is another VGAM31 host target gene. HRH1 BINDING SITE is HOST TARGET binding site found in the 3′ untranslated region of mRNA encoded by HRH1, corresponding to a HOST TARGET binding site such as BINDING SITE I, BINDING SITE II or BINDING SITE III. Table 2 illustrates the complementarity of the nucleotide sequences of HRH1 BINDING SITE, designated SEQ ID:780, to the nucleotide sequence of VGAM31 RNA, herein designated VGAM RNA, also designated SEQ ID:366.

Another function of VGAM31 is therefore inhibition of Histamine Receptor H1 (HRH1, Accession NM_{—}000861), a gene which stimulates the synthesis of inositol phosphate. Accordingly, utilities of VGAM31 include diagnosis, prevention and treatment of diseases and clinical conditions associated with HRH1. The function of HRH1 has been established by previous studies. Histamine is a ubiquitous messenger molecule released from mast cells, enterochromaffin-like cells, and neurons. Its various actions are mediated by 3 pharmacologically defined receptors termed the H1, H2 (OMIM Ref. No. 142703), and H3 (OMIM Ref. No. 604525) receptors. The H1 receptor was the first member of this family to be pharmacologically defined with the design of selective antagonists, the ‘antihistamines,’ which are used to treat allergic and inflammatory reactions. The H1 receptor is expressed by various peripheral tissues, such as smooth muscle, and by neurons in the brain, where histamine may be involved in the control of wakefulness, mood, and hormone secretion. Yamashita et al. (1991) cloned a bovine H1 receptor cDNA and established its nucleotide sequence. Its homology with the corresponding sequence of other receptors confirmed that it belongs to the super family of receptors coupled with G proteins with 7 putative transmembrane domains. In addition to their expression in neuronal, gastric, and muscular tissue, the G protein-coupled receptors HRH1 and HRH2 are also expressed on T-helper lymphocytes and trigger different intracellular events upon activation. Using flow cytometric analysis, Jutel et al. (2001) demonstrated that histamine binds more strongly to Th1 than to Th2 cells. Flow cytometry and RT-PCR analysis showed that HRH1 is predominantly expressed on Th1 cells in an IL3 (OMIM Ref. No. 147740)-up-regulatable manner, while HRH2 is predominant on Th2 cells. Stimulation of naive, CD45RA+ (see OMIM Ref. No. 151460) T cells with IL12 (OMIM Ref. No. 161560) resulted in preferential expression of HRH1, but stimulation with IL4 (OMIM Ref. No. 147780) resulted in suppressed expression of HRH1, demonstrating that mature CD45RO+ Th1 and Th2 lymphocytes preferentially but not exclusively express HRH1 and HRH2, and that HRH1 and HRH2 are regulated by cytokines present in the immune environment. Histamine stimulation of Th1 cells resulted in significant calcium flux that could be blocked by an HRH1 antagonist, while stimulation of Th2 cells led to cAMP formation that could be blocked by an HRH2, but not an HRH1, antagonist. Furthermore, histamine enhanced Th1 but inhibited Th2 responses to anti-CD3. Histamine also enhanced peripheral blood mononuclear cell responses in sensitized individuals to a predominantly Th1 antigen, but suppressed responses to Th2 allergens. Animal model experiments lend further support to the function of HRH1. Ma et al. (2002) noted that pertussis toxin (PTX) elicits a range of responses in mice, including sensitization to vasoactive amines (VAAS) and increased vascular permeability subsequent to PTX-induced changes in vascular endothelial cells. Susceptible mouse strains die from hypotensive and hypovolemic shock on vasoactive amine challenge, whereas resistant strains do not. This hypersensitivity is controlled by an autosomal dominant locus, designated Bphs, localized to mouse chromosome 6. Using positional cloning, Ma et al. (2002) linked the Bphs locus to Hrh1. Mice lacking Hrh1 were protected from VAAS hypersensitivity, as well as from experimental allergic encephalomyelitis and experimental autoimmune orchitis. Sequence analysis showed that leu263-to-pro (L263P), met313-to-val (M313V), and ser331-to-pro (S331P) polymorphisms were associated with resistance to vasoactive amine challenge. The authors concluded that these Hrh1 alleles control both the autoimmune T-cell and vascular responses regulated by histamine after PTX sensitization.

It is appreciated that the abovementioned animal model for HRH1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Jutel, M.; Watanabe, T.; Klunker, S.; Akdis, M.; Thomet, O. A. R.; Malolepszy, J.; Zak-Nejmark, T.; Koga, R.; Kobayashi, T.; Blaser, K.; Akdis, C. A.: Histamine regulates T-cell and antibody responses by differential expression of H1 and H2 receptors. Nature 413: 420-425, 2001.; and
Ma, R. Z.; Gao, J.; Meeker, N. D.; Fillmore, P. D.; Tung, K. S. K.; Watanabe, T.; Zachary, J. F.; Offner, H.; Blankenhorn, E. P.; Teuscher, C.: Identification of Bphs, an autoimmune di.

Further studies establishing the function and utilities of HRH1 are found in John Hopkins OMIM database record ID 600167, and in sited publications numbered 179 and 1798-1800 listed in the bibliography section hereinbelow, which are also hereby incorporated by reference. Mitogen-activated Protein Kinase 14 (MAPK14, Accession NM_{—}001315) is another VGAM31 host target gene. MAPK14 BINDING SITE1 through MAPK14 BINDING SITE3 are HOST TARGET binding sites found in untranslated regions of mRNA encoded by MAPK14, corresponding to HOST TARGET binding sites such as BINDING SITE I, BINDING SITE II or BINDING SITE III. Table 2 illustrates the complementarity of the nucleotide sequences of MAPK14 BINDING SITE1 through MAPK14 BINDING SITE3, designated SEQ ID:818, SEQ ID:2465 and SEQ ID:2466 respectively, to the nucleotide sequence of VGAM31 RNA, herein designated VGAM RNA, also designated SEQ ID:366.

Another function of VGAM31 is therefore inhibition of Mitogen-activated Protein Kinase 14 (MAPK14, Accession NM_{—}001315), a gene which is important for cytokine production; responds to changes in extracellular osmolarity. Accordingly, utilities of VGAM31 include diagnosis, prevention and treatment of diseases and clinical conditions associated with MAPK14. The function of MAPK14 has been established by previous studies. Tamura et al. (2000) investigated a role for Mapk14 in mouse development and physiology by targeted disruption of the Mapk14 gene. Whereas some Mapk14 −/− embryos died between embryonic days 11.5 and 12.5, those that developed past this stage had normal morphology but were anemic, owing to failed definitive erythropoiesis caused by diminished expression of the erythropoietin gene (EPO; 133170). Since Mapk14-deficient hematopoietic stem cells reconstituted lethally irradiated hosts, Mapk14 function is not required downstream of the Epo receptor (EPOR; 133171). Inhibition of MAPK14 activity also interfered with stabilization of EPO mRNA in human hepatoma cells undergoing hypoxic stress. The authors concluded that MAPK14 plays a critical role linking developmental and stress-induced erythropoiesis through regulation of EPO expression. Using a yeast 2-hybrid screen of gastrointestinal tract tissue with p38-alpha as the bait, Ge et al. (2002) isolated multiple clones encoding TAB1 (OMIM Ref. No. 602615). Immunoprecipitation and GST pull-down analyses indicated that TAB1 interacts with p38-alpha, but not with other MAPKs, with or without treatment with TNF. Immunoblot analysis showed that coexpression of TAB1 and p38-alpha enhanced autophosphorylation of p38-alpha even in the presence of dominant-negative forms of MAP2Ks (e.g., MAP2K3; 602315) and TAK1 (MAP3K7; 602614). The amino acids between positions 373 and 418 of TAB1 were found to be required for phosphorylation of p38-alpha. Expression of TLR2 (OMIM Ref. No. 603028) caused p38-alpha phosphorylation in the presence or absence of inhibitors, whereas p38-alpha phosphorylation after stimulation of TLR4 (OMIM Ref. No. 603030) could be inhibited by mutant TAB1, suggesting that activation of p38-alpha can be TAB1 dependent or independent. Immunoblot analysis detected the formation of TRAF6 (OMIM Ref. No. 602355)-TAB1-p38-alpha complexes. Formation of these complexes could be enhanced by stimulation with lipopolysaccharide. Ge et al. (2002) concluded that activation of p38-alpha by a nonenzymatic adaptor protein such as TAB1 may be an important alternative activation pathway operating in parallel with kinase cascades in regulating intracellular signaling

Full details of the abovementioned studies are described in the following publications, the disclosure of which are hereby incorporated by reference:
Tamura, K.; Sudo, T.; Senftleben, U.; Dadak, A. M.; Johnson, R.; Karin, M.: Requirement for p38-alpha in erythropoietin expression: a role for stress kinases in erythropoiesis. Cell 102: 221-231, 2000.; and
Ge, B.; Gram, H.; Di Padova, F.; Huang, B.; New, L.; Ulevitch, R. J.; Luo, Y.; Han, J.: MAPKK-independent activation of p38-alpha mediated by TAB1-dependent autophosphorylation of p38-alp.

Further studies establishing the function and utilities of MAPK14 are found in John Hopkins OMIM database record ID 600289, and in sited publications numbered 2292, 2293, 2294-230 and 2541 listed in the bibliography section hereinbelow, which are also hereby incorporated by reference. Single-minded Homolog 1 (Drosophila) (SIM1, Accession NM_{—}005068) is another VGAM31 host target gene. SIM1 BINDING SITE is HOST TARGET binding site found in the 3′ untranslated region of mRNA encoded by SIM1, corresponding to a HOST TARGET binding site such as BINDING SITE I, BINDING SITE II or BINDING SITE III. Table 2 illustrates the complementarity of the nucleotide sequences of SIM1 BINDING SITE, designated SEQ ID:1181, to the nucleotide sequence of VGAM31 RNA, herein designated VGAM RNA, also designated SEQ ID:366.

Another function of VGAM31 is therefore inhibition of Single-minded Homolog 1 (Drosophila) (SIM1, Accession NM_{—}005068), a gene which may have pleiotropic effects during embryogenesis and in the adult. Accordingly, utilities of VGAM31 include diagnosis, prevention and treatment of diseases and clinical conditions associated with SIM1. The function of SIM1 has been established by previous studies. Studies of mice and humans have revealed a number of genes that when mutated result in severe obesity. Holder et al. (2000) studied a girl with early-onset obesity and a balanced translocation between 1p22.1 and 6q16.2. At 67 months of age she weighed 47.5 kg (+9.3 SD) and was 127.2 cm tall (+3.2 SD); her weight for height was +6.3 SD. The child displayed an aggressive, voracious appetite, and the obesity was thought to be due to high intake, since measured energy expenditure was normal. Holder et al. (2000) cloned and sequenced both translocation breakpoints. The translocation did not appear to affect any transcription unit on 1p, but it disrupted the SIM1 gene on 6q, separating the 5-prime promoter region and the bHLH domain from the 3-prime PAS and putative transcriptional regulation domains. The transcriptional targets of SIM1 were not known. Mouse Sim1 is expressed in the developing kidney and central nervous system and is essential for formation of the supraoptic and paraventricular (PVN) nuclei of the hypothalamus. Previous neuroanatomic and pharmacologic studies had implicated the PVN in the regulation of body weight: PVN neurons express the melanocortin-4 receptor (MC4R; 155541) and appear to be physiologic targets of alpha-melanocyte-stimulating hormone (OMIM Ref. No. 176830), which inhibits food intake. Holder et al. (2000) hypothesized that haploinsufficiency of SIM1, possibly acting upstream or downstream of MC4R in the PVN, was responsible for severe obesity in their patient. Animal model experiments lend further support to the function of SIM1. Mice homozygous for a null allele of Sim1 (Sim1 −/−) lack a paraventricular nucleus (PVN) and die perinatally. In contrast, Michaud et al. (2001) showed that Sim1 heterozygous mice were viable but developed early-onset obesity, with increased linear growth, hyperinsulinemia, and hyperleptinemia. Sim1+/− mice were hyperphagic but their energy expenditure was not decreased, distinguishing them from other mouse models of early-onset obesity such as deficiencies in leptin (OMIM Ref. No. 164160) and melanocortin receptor-4 (OMIM Ref. No. 155541). Quantitative histologic comparison with normal littermates showed that the PVN of Sim1 +/−mice contains on average 24% fewer cells without a selective loss of any identifiable major cell type. Since acquired lesions in the PVN also induce increased appetite without a decrease in energy expenditure, the authors proposed that abnormalities of PVN development may cause the obesity of Sim1 +/−mice.

It is appreciated that the abovementioned animal model for SIM1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Holder, J. L., Jr.; Butte, N. F.; Zinn, A. R.: Profound obesity associated with a balanced translocation that disrupts the SIM1 gene. Hum. Molec. Genet. 9: 101-108, 2000.; and
Michaud, J. L.; Boucher, F.; Melnyk, A.; Gauthier, F.; Goshu, E.; Levy, E.; Mitchell, G. A.; Himms-Hagen, J.; Fan, C.-M.: Sim1 haploinsufficiency causes hyperphagia, obesity and redu.

Further studies establishing the function and utilities of SIM1 are found in John Hopkins OMIM database record ID 603128, and in sited publications numbered 12 and 129-128 listed in the bibliography section hereinbelow, which are also hereby incorporated by reference. Chromosome 11 Open Reading Frame 25 (C11orf25, Accession NM_{—}031418) is another VGAM31 host target gene. C11orf25 BINDING SITE is HOST TARGET binding site found in the 3′ untranslated region of mRNA encoded by C11orf25, corresponding to a HOST TARGET binding site such as BINDING SITE I, BINDING SITE II or BINDING SITE III. Table 2 illustrates the complementarity of the nucleotide sequences of C11orf25 BINDING SITE, designated SEQ ID:2193, to the nucleotide sequence of VGAM31 RNA, herein designated VGAM RNA, also designated SEQ ID:366.

Another function of VGAM31 is therefore inhibition of Chromosome 11 Open Reading Frame 25 (C11orf25, Accession NM_{—}031418). Accordingly, utilities of VGAM31 include diagnosis, prevention and treatment of diseases and clinical conditions associated with C11orf25. DKFZP564D116 (Accession XM_{—}051050) is another VGAM31 host target gene. DKFZP564D116 BINDING SITE is HOST TARGET binding site found in the 3′ untranslated region of mRNA encoded by DKFZP564D116, corresponding to a HOST TARGET binding site such as BINDING SITE I, BINDING SITE II or BINDING SITE III. Table 2 illustrates the complementarity of the nucleotide sequences of DKFZP564D116 BINDING SITE, designated SEQ ID:2938, to the nucleotide sequence of VGAM31 RNA, herein designated VGAM RNA, also designated SEQ ID:366.

Another function of VGAM31 is therefore inhibition of DKFZP564D116 (Accession XM_{—}051050). Accordingly, utilities of VGAM31 include diagnosis, prevention and treatment of diseases and clinical conditions associated with DKFZP564D116. Down Syndrome Critical Region Gene 6 (DSCR6, Accession NM_{—}018962) is another VGAM31 host target gene. DSCR6 BINDING SITE is HOST TARGET binding site found in the 3′ untranslated region of mRNA encoded by DSCR6, corresponding to a HOST TARGET binding site such as BINDING SITE I, BINDING SITE II or BINDING SITE III. Table 2 illustrates the complementarity of the nucleotide sequences of DSCR6 BINDING SITE, designated SEQ ID:1866, to the nucleotide sequence of VGAM31 RNA, herein designated VGAM RNA, also designated SEQ ID:366.

Another function of VGAM31 is therefore inhibition of Down Syndrome Critical Region Gene 6 (DSCR6, Accession NM_{—}018962). Accordingly, utilities of VGAM31 include diagnosis, prevention and treatment of diseases and clinical conditions associated with DSCR6. FLJ10006 (Accession XM_{—}087073) is another VGAM31 host target gene. FLJ10006 BINDING SITE is HOST TARGET binding site found in the 3′ untranslated region of mRNA encoded by FLJ10006, corresponding to a HOST TARGET binding site such as BINDING SITE I, BINDING SITE II or BINDING SITE III. Table 2 illustrates the complementarity of the nucleotide sequences of FLJ10006 BINDING SITE, designated SEQ ID:3152, to the nucleotide sequence of VGAM31 RNA, herein designated VGAM RNA, also designated SEQ ID:366.

Another function of VGAM31 is therefore inhibition of FLJ10006 (Accession XM_{—}087073). Accordingly, utilities of VGAM31 include diagnosis, prevention and treatment of diseases and clinical conditions associated with FLJ10006. HTPAP (Accession NM_{—}032483) is another VGAM31 host target gene. HTPAP BINDING SITE is HOST TARGET binding site found in the 3′ untranslated region of mRNA encoded by HTPAP, corresponding to a HOST TARGET binding site such as BINDING SITE I, BINDING SITE II or BINDING SITE III. Table 2 illustrates the complementarity of the nucleotide sequences of HTPAP BINDING SITE, designated SEQ ID:2255, to the nucleotide sequence of VGAM31 RNA, herein designated VGAM RNA, also designated SEQ ID:366.

Another function of VGAM31 is therefore inhibition of HTPAP (Accession NM_{—}032483). Accordingly, utilities of VGAM31 include diagnosis, prevention and treatment of diseases and clinical conditions associated with HTPAP. KIAA0979 (Accession NM_{—}015032) is another VGAM31 host target gene. KIAA0979 BINDING SITE is HOST TARGET binding site found in the 3′ untranslated region of mRNA encoded by KIAA0979, corresponding to a HOST TARGET binding site such as BINDING SITE I, BINDING SITE II or BINDING SITE III. Table 2 illustrates the complementarity of the nucleotide sequences of KIAA0979 BINDING SITE, designated SEQ ID: 1601, to the nucleotide sequence of VGAM31 RNA, herein designated VGAM RNA, also designated SEQ ID:366.

Another function of VGAM31 is therefore inhibition of KIAA0979 (Accession NM_{—}015032). Accordingly, utilities of VGAM31 include diagnosis, prevention and treatment of diseases and clinical conditions associated with KIAA0979. KIAA1634 (Accession XM_{—}032749) is another VGAM31 host target gene. KIAA1634 BINDING SITE is HOST TARGET binding site found in the 3′ untranslated region of mRNA encoded by KIAA1634, corresponding to a HOST TARGET binding site such as BINDING SITE I, BINDING SITE II or BINDING SITE III. Table 2 illustrates the complementarity of the nucleotide sequences of KIAA1634 BINDING SITE, designated SEQ ID:2657, to the nucleotide sequence of VGAM31 RNA, herein designated VGAM RNA, also designated SEQ ID:366.

Another function of VGAM31 is therefore inhibition of KIAA1634 (Accession XM_{—}032749). Accordingly, utilities of VGAM31 include diagnosis, prevention and treatment of diseases and clinical conditions associated with KIAA1634. PTD012 (Accession NM_{—}014039) is another VGAM31 host target gene. PTD012 BINDING SITE is HOST TARGET binding site found in the 3′ untranslated region of mRNA encoded by PTD012, corresponding to a HOST TARGET binding site such as BINDING SITE I, BINDING SITE II or BINDING SITE III. Table 2 illustrates the complementarity of the nucleotide sequences of PTD012 BINDING SITE, designated SEQ ID: 1465, to the nucleotide sequence of VGAM31 RNA, herein designated VGAM RNA, also designated SEQ ID:366.

Another function of VGAM31 is therefore inhibition of PTD012 (Accession NM_{—}014039). Accordingly, utilities of VGAM31 include diagnosis, prevention and treatment of diseases and clinical conditions associated with PTD012. SAM Domain and HD Domain 1 (SAMHD1, Accession XM_{—}028704) is another VGAM31 host target gene. SAMHD1 BINDING SITE is HOST TARGET binding site found in the 3′ untranslated region of mRNA encoded by SAMHD1, corresponding to a HOST TARGET binding site such as BINDING SITE I, BINDING SITE II or BINDING SITE III. Table 2 illustrates the complementarity of the nucleotide sequences of SAMHD1 BINDING SITE, designated SEQ ID:2593, to the nucleotide sequence of VGAM31 RNA, herein designated VGAM RNA, also designated SEQ ID:366.

Another function of VGAM31 is therefore inhibition of SAM Domain and HD Domain 1 (SAMHD1, Accession XM_{—}028704). Accordingly, utilities of VGAM31 include diagnosis, prevention and treatment of diseases and clinical conditions associated with SAMHD1. LOC196758 (Accession XM_{—}113600) is another VGAM31 host target gene. LOC196758 BINDING SITE is HOST TARGET binding site found in the 3′ untranslated region of mRNA encoded by LOC196758, corresponding to a HOST TARGET binding site such as BINDING SITE I, BINDING SITE II or BINDING SITE III. Table 2 illustrates the complementarity of the nucleotide sequences of LOC196758 BINDING SITE, designated SEQ ID:3410, to the nucleotide sequence of VGAM31 RNA, herein designated VGAM RNA, also designated SEQ ID:366.

Another function of VGAM31 is therefore inhibition of LOC196758 (Accession XM_{—}113600). Accordingly, utilities of VGAM31 include diagnosis, prevention and treatment of diseases and clinical conditions associated with LOC196758. LOC219790 (Accession XM_{—}166124) is another VGAM31 host target gene. LOC219790 BINDING SITE is HOST TARGET binding site found in the 3′ untranslated region of mRNA encoded by LOC219790, corresponding to a HOST TARGET binding site such as BINDING SITE I, BINDING SITE II or BINDING SITE III. Table 2 illustrates the complementarity of the nucleotide sequences of LOC219790 BINDING SITE, designated SEQ ID:3520, to the nucleotide sequence of VGAM31 RNA, herein designated VGAM RNA, also designated SEQ ID:366.

Another function of VGAM31 is therefore inhibition of LOC219790 (Accession XM_{—}166124). Accordingly, utilities of VGAM31 include diagnosis, prevention and treatment of diseases and clinical conditions associated with LOC219790. FIG. 1 further provides a conceptual description of a novel bioinformatically detected viral gene of the present invention, referred to here as Viral Genomic Address Messenger 32 (VGAM32) viral gene, which modulates expression of respective host target genes thereof, the function and utility of which host target genes is known in the art.

VGAM32 is a novel bioinformatically detected regulatory, non protein coding, viral micro RNA (miRNA) gene. The method by which VGAM32 was detected is described hereinabove with reference to FIGS. 1-8.

VGAM32 gene, herein designated VGAM GENE, is a viral gene contained in the genome of Vaccinia Virus. VGAM32 host target gene, herein designated VGAM HOST TARGET GENE, is a human gene contained in the human genome.

VGAM32 gene encodes a VGAM32 precursor RNA, herein designated VGAM PRECURSOR RNA. Similar to other miRNA genes, and unlike most ordinary genes, VGAM32 precursor RNA does not encode a protein. A nucleotide sequence identical or highly similar to the nucleotide sequence of VGAM32 precursor RNA is designated SEQ ID:18, and is provided hereinbelow with reference to the sequence listing part. Nucleotide sequence SEQ ID:18 is located at position 190813 relative to the genome of Vaccinia Virus.

VGAM32 precursor RNA folds onto itself, forming VGAM32 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM32 folded precursor RNA into VGAM32 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM32 RNA is designated SEQ ID:367, and is provided hereinbelow with reference to the sequence listing part.

VGAM32 host target gene, herein designated VGAM HOST TARGET GENE, encodes a corresponding messenger RNA, VGAM32 host target RNA, herein designated VGAM HOST TARGET RNA. VGAM32 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM32 RNA, herein designated VGAM RNA, binds complementarily to one or more host target binding sites located in untranslated regions of VGAM32 host target RNA, herein designated VGAM HOST TARGET RNA. This complementary binding is due to the fact that the nucleotide sequence of VGAM32 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM32 RNA, herein designated VGAM RNA, may have a different number of host target binding sites in untranslated regions of a VGAM32 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM32 RNA, herein designated VGAM RNA, to host target binding sites on VGAM32 host target RNA, herein designated VGAM HOST TARGET RNA, such as BINDING SITE I, BINDING SITE II and BINDING SITE III, inhibits translation of VGAM32 host target RNA into VGAM32 host target protein, herein designated VGAM HOST TARGET PROTEIN. VGAM host target protein is therefore outlined by a broken line.

It is appreciated that VGAM32 host target gene, herein designated VGAM HOST TARGET GENE, in fact represents a plurality of VGAM32 host target genes. The mRNA of each one of this plurality of VGAM32 host target genes comprises one or more host target binding sites, each having a nucleotide sequence which is at least partly complementary to VGAM32 RNA, herein designated VGAM RNA, and which when bound by VGAM32 RNA causes inhibition of translation of respective one or more VGAM32 host target proteins.

It is further appreciated by one skilled in the art that the mode of translational inhibition illustrated by FIG. 1 with specific reference to translational inhibition exerted by VGAM32 gene, herein designated VGAM GENE, on one or more VGAM32 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM32 is inhibition of expression of host target genes, as part of a novel viral mechanism of attacking a host. Accordingly, utilities of VGAM32 include diagnosis, prevention and treatment of viral infection by Vaccinia Virus. Specific functions, and accordingly utilities, of VGAM32 correlate with, and may be deduced from, the identity of the host target genes which VGAM32 binds and inhibits, and the function of these host target genes, as elaborated hereinbelow.

Nucleotide sequences of the VGAM32 precursor RNA, herein designated VGAM PRECURSOR RNA, and of the ‘diced’ VGAM32 RNA, herein designated VGAM RNA, and a schematic representation of the secondary folding of VGAM32 folded precursor RNA, herein designated VGAM FOLDED PRECURSOR RNA, of VGAM32 are further described hereinbelow with reference to Table 1.

Nucleotide sequences of host target binding sites, such as BINDING SITE-I, BINDING SITE-II and BINDING SITE-III of FIG. 1, found on VGAM32 host target RNA, and schematic representation of the complementarity of each of these host target binding sites to VGAM32 RNA, herein designated VGAM RNA, are described hereinbelow with reference to Table 2.

As mentioned hereinabove with reference to FIG. 1, a function of VGAM32 gene, herein designated VGAM is inhibition of expression of VGAM32 target genes. It is appreciated that specific functions, and accordingly utilities, of VGAM32 correlate with, and may be deduced from, the identity of the target genes which VGAM32 binds and inhibits, and the function of these target genes, as elaborated hereinbelow.

Calpain 2, (m/II) Large Subunit (CAPN2, Accession NM_{—}001748) is a VGAM32 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM32 RNA, herein designated VGAM RNA, also designated SEQ ID:367.

A function of VGAM32 is therefore inhibition of Calpain 2, (m/II) Large Subunit (CAPN2, Accession NM_{—}001748). Accordingly, utilities of VGAM32 include diagnosis, prevention and treatment of diseases and clinical conditions associated with CAPN2. Dishevelled Associated Activator of Morphogenesis 2 (DAAM2, Accession XM_{—}166434) is another VGAM32 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM32 RNA, herein designated VGAM RNA, also designated SEQ ID:367.

Another function of VGAM32 is therefore inhibition of Dishevelled Associated Activator of Morphogenesis 2 (DAAM2, Accession XM_{—}166434), a gene which controls cell polarity and movement during development. Accordingly, utilities of VGAM32 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM32 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM32 RNA, herein designated VGAM RNA, also designated SEQ ID:367.

Another function of VGAM32 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM32 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM32 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM32 RNA, herein designated VGAM RNA, also designated SEQ ID:367.

Another function of VGAM32 is therefore inhibition of KIAA0475 (Accession NM_{—}014864). Accordingly, utilities of VGAM32 include diagnosis, prevention and treatment of diseases and clinical conditions associated with KIAA0475. LOC222671 (Accession XM_{—}167094) is another VGAM32 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM32 RNA, herein designated VGAM RNA, also designated SEQ ID:367.

Another function of VGAM32 is therefore inhibition of LOC222671 (Accession XM_{—}167094). Accordingly, utilities of VGAM32 include diagnosis, prevention and treatment of diseases and clinical conditions associated with LOC222671. LOC254901 (Accession XM_{—}171878) is another VGAM32 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM32 RNA, herein designated VGAM RNA, also designated SEQ ID:367.

Another function of VGAM32 is therefore inhibition of LOC254901 (Accession XM_{—}171878). Accordingly, utilities of VGAM32 include diagnosis, prevention and treatment of diseases and clinical conditions associated with LOC254901. LOC257615 (Accession XM_{—}175293) is another VGAM32 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM32 RNA, herein designated VGAM RNA, also designated SEQ ID:367.

Another function of VGAM32 is therefore inhibition of LOC257615 (Accession XM_{—}175293). Accordingly, utilities of VGAM32 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 33 (VGAM33) viral gene, which modulates expression of respective host target genes thereof, the function and utility of which host target genes is known in the art.

VGAM33 is a novel bioinformatically detected regulatory, non protein coding, viral micro RNA (miRNA) gene. The method by which VGAM33 was detected is described hereinabove with reference to FIGS. 1-8.

VGAM33 gene, herein designated VGAM GENE, is a viral gene contained in the genome of Vaccinia Virus. VGAM33 host target gene, herein designated VGAM HOST TARGET GENE, is a human gene contained in the human genome.

VGAM33 gene encodes a VGAM33 precursor RNA, herein designated VGAM PRECURSOR RNA. Similar to other miRNA genes, and unlike most ordinary genes, VGAM33 precursor RNA does not encode a protein. A nucleotide sequence identical or highly similar to the nucleotide sequence of VGAM33 precursor RNA is designated SEQ ID:19, and is provided hereinbelow with reference to the sequence listing part. Nucleotide sequence SEQ ID:19 is located at position 190813 relative to the genome of Vaccinia Virus.

VGAM33 precursor RNA folds onto itself, forming VGAM33 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM33 folded precursor RNA into VGAM33 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM33 RNA is designated SEQ ID:368, and is provided hereinbelow with reference to the sequence listing part.

VGAM33 host target gene, herein designated VGAM HOST TARGET GENE, encodes a corresponding messenger RNA, VGAM33 host target RNA, herein designated VGAM HOST TARGET RNA. VGAM33 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM33 RNA, herein designated VGAM RNA, binds complementarily to one or more host target binding sites located in untranslated regions of VGAM33 host target RNA, herein designated VGAM HOST TARGET RNA. This complementary binding is due to the fact that the nucleotide sequence of VGAM33 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM33 RNA, herein designated VGAM RNA, may have a different number of host target binding sites in untranslated regions of a VGAM33 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM33 RNA, herein designated VGAM RNA, to host target binding sites on VGAM33 host target RNA, herein designated VGAM HOST TARGET RNA, such as BINDING SITE I, BINDING SITE II and BINDING SITE III, inhibits translation of VGAM33 host target RNA into VGAM33 host target protein, herein designated VGAM HOST TARGET PROTEIN. VGAM host target protein is therefore outlined by a broken line.

It is appreciated that VGAM33 host target gene, herein designated VGAM HOST TARGET GENE, in fact represents a plurality of VGAM33 host target genes. The mRNA of each one of this plurality of VGAM33 host target genes comprises one or more host target binding sites, each having a nucleotide sequence which is at least partly complementary to VGAM33 RNA, herein designated VGAM RNA, and which when bound by VGAM33 RNA causes inhibition of translation of respective one or more VGAM33 host target proteins.

It is further appreciated by one skilled in the art that the mode of translational inhibition illustrated by FIG. 1 with specific reference to translational inhibition exerted by VGAM33 gene, herein designated VGAM GENE, on one or more VGAM33 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM33 is inhibition of expression of host target genes, as part of a novel viral mechanism of attacking a host. Accordingly, utilities of VGAM33 include diagnosis, prevention and treatment of viral infection by Vaccinia Virus. Specific functions, and accordingly utilities, of VGAM33 correlate with, and may be deduced from, the identity of the host target genes which VGAM33 binds and inhibits, and the function of these host target genes, as elaborated hereinbelow.

Nucleotide sequences of the VGAM33 precursor RNA, herein designated VGAM PRECURSOR RNA, and of the ‘diced’ VGAM33 RNA, herein designated VGAM RNA, and a schematic representation of the secondary folding of VGAM33 folded precursor RNA, herein designated VGAM FOLDED PRECURSOR RNA, of VGAM33 are further described hereinbelow with reference to Table 1.

Nucleotide sequences of host target binding sites, such as BINDING SITE-I, BINDING SITE-II and BINDING SITE-III of FIG. 1, found on VGAM33 host target RNA, and schematic representation of the complementarity of each of these host target binding sites to VGAM33 RNA, herein designated VGAM RNA, are described hereinbelow with reference to Table 2.

As mentioned hereinabove with reference to FIG. 1, a function of VGAM33 gene, herein designated VGAM is inhibition of expression of VGAM33 target genes. It is appreciated that specific functions, and accordingly utilities, of VGAM33 correlate with, and may be deduced from, the identity of the target genes which VGAM33 binds and inhibits, and the function of these target genes, as elaborated hereinbelow.

Calpain 2, (m/II) Large Subunit (CAPN2, Accession NM_{—}001748) is a VGAM33 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM33 RNA, herein designated VGAM RNA, also designated SEQ ID:368.

A function of VGAM33 is therefore inhibition of Calpain 2, (m/II) Large Subunit (CAPN2, Accession NM_{—}001748). Accordingly, utilities of VGAM33 include diagnosis, prevention and treatment of diseases and clinical conditions associated with CAPN2. Dishevelled Associated Activator of Morphogenesis 2 (DAAM2, Accession XM_{—}166434) is another VGAM33 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM33 RNA, herein designated VGAM RNA, also designated SEQ ID:368.

Another function of VGAM33 is therefore inhibition of Dishevelled Associated Activator of Morphogenesis 2 (DAAM2, Accession XM_{—}166434), a gene which controls cell polarity and movement during development. Accordingly, utilities of VGAM33 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM33 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM33 RNA, herein designated VGAM RNA, also designated SEQ ID:368.

Another function of VGAM33 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM33 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM33 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM33 RNA, herein designated VGAM RNA, also designated SEQ ID:368.

Another function of VGAM33 is therefore inhibition of KIAA0475 (Accession NM_{—}014864). Accordingly, utilities of VGAM33 include diagnosis, prevention and treatment of diseases and clinical conditions associated with KIAA0475. LOC222671 (Accession XM_{—}167094) is another VGAM33 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM33 RNA, herein designated VGAM RNA, also designated SEQ ID:368.

Another function of VGAM33 is therefore inhibition of LOC222671 (Accession XM_{—}167094). Accordingly, utilities of VGAM33 include diagnosis, prevention and treatment of diseases and clinical conditions associated with LOC222671. LOC254901 (Accession XM_{—}171878) is another VGAM33 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM33 RNA, herein designated VGAM RNA, also designated SEQ ID:368.

Another function of VGAM33 is therefore inhibition of LOC254901 (Accession XM_{—}171878). Accordingly, utilities of VGAM33 include diagnosis, prevention and treatment of diseases and clinical conditions associated with LOC254901. LOC257615 (Accession XM_{—}175293) is another VGAM33 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM33 RNA, herein designated VGAM RNA, also designated SEQ ID:368.

Another function of VGAM33 is therefore inhibition of LOC257615 (Accession XM_{—}175293). Accordingly, utilities of VGAM33 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 34 (VGAM34) viral gene, which modulates expression of respective host target genes thereof, the function and utility of which host target genes is known in the art.

VGAM34 is a novel bioinformatically detected regulatory, non protein coding, viral micro RNA (miRNA) gene. The method by which VGAM34 was detected is described hereinabove with reference to FIGS. 1-8.

VGAM34 gene, herein designated VGAM GENE, is a viral gene contained in the genome of Vaccinia Virus. VGAM34 host target gene, herein designated VGAM HOST TARGET GENE, is a human gene contained in the human genome.

VGAM34 gene encodes a VGAM34 precursor RNA, herein designated VGAM PRECURSOR RNA. Similar to other miRNA genes, and unlike most ordinary genes, VGAM34 precursor RNA does not encode a protein. A nucleotide sequence identical or highly similar to the nucleotide sequence of VGAM34 precursor RNA is designated SEQ ID:20, and is provided hereinbelow with reference to the sequence listing part. Nucleotide sequence SEQ ID:20 is located at position 188778 relative to the genome of Vaccinia Virus.

VGAM34 precursor RNA folds onto itself, forming VGAM34 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM34 folded precursor RNA into VGAM34 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM34 RNA is designated SEQ ID:369, and is provided hereinbelow with reference to the sequence listing part.

VGAM34 host target gene, herein designated VGAM HOST TARGET GENE, encodes a corresponding messenger RNA, VGAM34 host target RNA, herein designated VGAM HOST TARGET RNA. VGAM34 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM34 RNA, herein designated VGAM RNA, binds complementarily to one or more host target binding sites located in untranslated regions of VGAM34 host target RNA, herein designated VGAM HOST TARGET RNA. This complementary binding is due to the fact that the nucleotide sequence of VGAM34 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM34 RNA, herein designated VGAM RNA, may have a different number of host target binding sites in untranslated regions of a VGAM34 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM34 RNA, herein designated VGAM RNA, to host target binding sites on VGAM34 host target RNA, herein designated VGAM HOST TARGET RNA, such as BINDING SITE I, BINDING SITE II and BINDING SITE III, inhibits translation of VGAM34 host target RNA into VGAM34 host target protein, herein designated VGAM HOST TARGET PROTEIN. VGAM host target protein is therefore outlined by a broken line.

It is appreciated that VGAM34 host target gene, herein designated VGAM HOST TARGET GENE, in fact represents a plurality of VGAM34 host target genes. The mRNA of each one of this plurality of VGAM34 host target genes comprises one or more host target binding sites, each having a nucleotide sequence which is at least partly complementary to VGAM34 RNA, herein designated VGAM RNA, and which when bound by VGAM34 RNA causes inhibition of translation of respective one or more VGAM34 host target proteins.

It is further appreciated by one skilled in the art that the mode of translational inhibition illustrated by FIG. 1 with specific reference to translational inhibition exerted by VGAM34 gene, herein designated VGAM GENE, on one or more VGAM34 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM34 is inhibition of expression of host target genes, as part of a novel viral mechanism of attacking a host. Accordingly, utilities of VGAM34 include diagnosis, prevention and treatment of viral infection by Vaccinia Virus. Specific functions, and accordingly utilities, of VGAM34 correlate with, and may be deduced from, the identity of the host target genes which VGAM34 binds and inhibits, and the function of these host target genes, as elaborated hereinbelow.

Nucleotide sequences of the VGAM34 precursor RNA, herein designated VGAM PRECURSOR RNA, and of the ‘diced’ VGAM34 RNA, herein designated VGAM RNA, and a schematic representation of the secondary folding of VGAM34 folded precursor RNA, herein designated VGAM FOLDED PRECURSOR RNA, of VGAM34 are further described hereinbelow with reference to Table 1.

Nucleotide sequences of host target binding sites, such as BINDING SITE-I, BINDING SITE-II and BINDING SITE-III of FIG. 1, found on VGAM34 host target RNA, and schematic representation of the complementarity of each of these host target binding sites to VGAM34 RNA, herein designated VGAM RNA, are described hereinbelow with reference to Table 2.

As mentioned hereinabove with reference to FIG. 1, a function of VGAM34 gene, herein designated VGAM is inhibition of expression of VGAM34 target genes. It is appreciated that specific functions, and accordingly utilities, of VGAM34 correlate with, and may be deduced from, the identity of the target genes which VGAM34 binds and inhibits, and the function of these target genes, as elaborated hereinbelow.

Adenylate Cyclase 6 (ADCY6, Accession NM_{—}015270) is a VGAM34 host target gene. ADCY6 BINDING SITE1 and ADCY6 BINDING SITE2 are HOST TARGET binding sites found in untranslated regions of mRNA encoded by ADCY6, corresponding to HOST TARGET binding sites such as BINDING SITE I, BINDING SITE II or BINDING SITE III. Table 2 illustrates the complementarity of the nucleotide sequences of ADCY6 BINDING SITE1 and ADCY6 BINDING SITE2, designated SEQ ID: 1617 and SEQ ID: 1933 respectively, to the nucleotide sequence of VGAM34 RNA, herein designated VGAM RNA, also designated SEQ ID:369.

A function of VGAM34 is therefore inhibition of Adenylate Cyclase 6 (ADCY6, Accession NM_{—}015270), a gene which this a membrane-bound, ca(2+)-inhabitable adenylyl cyclase (by similarity). Accordingly, utilities of VGAM34 include diagnosis, prevention and treatment of diseases and clinical conditions associated with ADCY6. The function of ADCY6 has been established by previous studies. By Southern blot analysis of somatic cell hybrid DNAs, Gaudin et al. (1994) mapped the ADCY6 gene to chromosome 12. Using isotopic in situ hybridization, Haber et al. (1994) mapped the ADCY6 gene to 12q12-q13. By fluorescence in situ hybridization, Edelhoff et al. (1995) confirmed the assignment of ADCY6 to 12q13 and demonstrated that the homologous mouse gene is located on chromosome 15 in the F region.

Full details of the abovementioned studies are described in the following publications, the disclosure of which are hereby incorporated by reference:
Edelhoff, S.; Villacres, E. C.; Storm, D. R.; Disteche, C. M.: Mapping of adenylyl cyclase genes type I, II, III, IV, V, and VI in mouse. Mammalian Genome 6: 111-113, 1995.; and
Gaudin, C.; Homcy, C. J.; Ishikawa, Y.: Mammalian adenylyl cyclase family members are randomly located on different chromosomes. Hum. Genet. 94: 527-529, 1994.

Further studies establishing the function and utilities of ADCY6 are found in John Hopkins OMIM database record ID 600294, and in sited publications numbered 85-8 and 2305 listed in the bibliography section hereinbelow, which are also hereby incorporated by reference. ATPase, Class I, Type 8B, Member 2 (ATP8B2, Accession XM_{—}036933) is another VGAM34 host target gene. ATP8B2 BINDING SITE is HOST TARGET binding site found in the 3′ untranslated region of mRNA encoded by ATP8B2, corresponding to a HOST TARGET binding site such as BINDING SITE I, BINDING SITE II or BINDING SITE III. Table 2 illustrates the complementarity of the nucleotide sequences of ATP8B2 BINDING SITE, designated SEQ ID:2716, to the nucleotide sequence of VGAM34 RNA, herein designated VGAM RNA, also designated SEQ ID:369.

Another function of VGAM34 is therefore inhibition of ATPase, Class I, Type 8B, Member 2 (ATP8B2, Accession XM_{—}036933). Accordingly, utilities of VGAM34 include diagnosis, prevention and treatment of diseases and clinical conditions associated with ATP8B2. Carcinoembryonic Antigen-related Cell Adhesion Molecule 1 (biliary glycoprotein) (CEACAM1, Accession NM_{—}001712) is another VGAM34 host target gene. CEACAM1 BINDING SITE is HOST TARGET binding site found in the 3′ untranslated region of mRNA encoded by CEACAM1, corresponding to a HOST TARGET binding site such as BINDING SITE I, BINDING SITE II or BINDING SITE III. Table 2 illustrates the complementarity of the nucleotide sequences of CEACAM1 BINDING SITE, designated SEQ ID:848, to the nucleotide sequence of VGAM34 RNA, herein designated VGAM RNA, also designated SEQ ID:369.

Another function of VGAM34 is therefore inhibition of Carcinoembryonic Antigen-related Cell Adhesion Molecule 1 (biliary glycoprotein) (CEACAM1, Accession NM_{—}001712), a gene which is a major effector of VEGF and may be a target for the inhibition of tumor angiogenesis. Accordingly, utilities of VGAM34 include diagnosis, prevention and treatment of diseases and clinical conditions associated with CEACAM1. The function of CEACAM1 has been established by previous studies. Ergun et al. (2000) showed that CEACAM1 exhibits angiogenic properties in in vitro and in vivo angiogenesis assays. CEACAM1 purified from granulocytes and endothelial cell media as well as recombinant CEACAM1 expressed in HEK293 cells stimulated proliferation, chemotaxis, and capillary-like tube formation of human microvascular endothelial cells. They increased vascularization of chick chorioallantoic membrane and potentiated the effects of VEGF165 (OMIM Ref. No. 192240). VEGF165 increased CEACAM1 expression at both the mRNA and the protein level. VEGF165-induced endothelial tube formation was blocked by a monoclonal CEACAM1 antibody. These data suggested that CEACAM1 is a major effector of VEGF in the early microvessel formation. Since CEACAM1 is expressed in tumor microvessels but not in large blood vessels, CEACAM1 may be a target for the inhibition of tumor angiogenesis. Following infection with Neisseria gonorrhea, there is a transient decline in circulating CD4 (OMIM Ref. No. 186940)-positive T lymphocytes that resolves after bacterial clearance. The gonococcus adheres to and is taken up by host cells through opacity-associated (Opa) proteins. Some Opa variants bind to heparan sulfate proteoglycans (HSPGs, e.g., SDC2; 142460), while others are specific for members of the CEACAM1/CD66 receptor family. CEACAM1 is the only member of this family that is expressed by lymphocytes and that contains a cytoplasmic ITIM (immuno receptor tyrosine-based inhibitory motif). Using flow cytometry, Boulton and Gray-Owen (2002) demonstrated that CEACAM1 expression is up-regulated after lymphocyte activation. Exposure to gonococci expressing the HSPG-specific Opa50 protein increased and exposure to CEACAM1-specific Opa52 gonococci or to anti-CEACAM1 antibody inhibited expression of the CD69 (OMIM Ref. No. 107273) activation marker on and proliferation by lymphocytes stimulated in vitro. The reduction in lymphocyte proliferation was not due to an increase in cell death. CEACAM1 associated with Opa52 also interacted with SHP1 (OMIM Ref. No. 176883) and SHP2 (OMIM Ref. No. 176876), presumably through its cytoplasmic ITIM. Boulton and Gray-Owen (2002) suggested that Opa52 engagement of the CEACAM1 coinhibitory receptor induces immunosuppression and may explain the failure of the host to develop a memory humoral response to N. gonorrhea infection due to a lack of T-cell help for B-cell activation. Animal model experiments lend further support to the function of CEACAM1. Poy et al. (2002) hypothesized that insulin stimulates phosphorylation of CEACAM1 which in turn leads to up-regulation of receptor-mediated insulin endocytosis and degradation in the hepatocyte. To test the hypothesis, they generated transgenic mice overexpressing in liver a dominant-negative phosphorylation-defective CEACAM1 mutant, S503A. Supporting their hypothesis, they found that S503A-CEACAM1 transgenic mice developed hyperinsulinemia resulting from impaired insulin clearance. The hyperinsulinemia caused secondary insulin resistance with impaired glucose tolerance and random, but not fasting, hyperglycemia. Transgenic mice developed visceral adiposity with increased amounts of plasma free fatty acids and plasma and hepatic triglycerides. These findings suggested a mechanism through which insulin signaling regulates insulin sensitivity by modulating hepatic insulin clearance.

It is appreciated that the abovementioned animal model for CEACAM1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ergun, S.; Kilic, N.; Ziegeler, G.; Hansen, A.; Nollau, P.; Gotze, J.; Wurmbach, J.-H.; Horst, A.; Weil, J.; Fernando, M.; Wagener, C.: CEA-related cell adhesion molecule 1: a potent angiogenic factor and a major effector of vascular endothelial growth factor. Molec. Cell 5: 311-320, 2000.; and
Boulton, I. C.; Gray-Owen, S. D.: Neisserial binding to CEACAM1 arrests the activation and proliferation of CD4+ T lymphocytes. Nature Immun. 3: 229-236, 2002.

Further studies establishing the function and utilities of CEACAM1 are found in John Hopkins OMIM database record ID 109770, and in sited publications numbered 2820, 2821, 2822-2824, 2467, 2825-2826, 47, 2464-2465, 2468, 282 and 2469 listed in the bibliography section hereinbelow, which are also hereby incorporated by reference. Carbohydrate (keratan sulfate Gal-6) Sulfotransferase 1 (CHST1, Accession NM_{—}003654) is another VGAM34 host target gene. CHST1 BINDING SITE is HOST TARGET binding site found in the 3′ untranslated region of mRNA encoded by CHST1, corresponding to a HOST TARGET binding site such as BINDING SITE I, BINDING SITE II or BINDING SITE III. Table 2 illustrates the complementarity of the nucleotide sequences of CHST1 BINDING SITE, designated SEQ ID: 1046, to the nucleotide sequence of VGAM34 RNA, herein designated VGAM RNA, also designated SEQ ID:369.

Another function of VGAM34 is therefore inhibition of Carbohydrate (keratan sulfate Gal-6) Sulfotransferase 1 (CHST1, Accession NM_{—}003654), a gene which may play a role in keratan sulfate biosynthesis in brain and cornea. Accordingly, utilities of VGAM34 include diagnosis, prevention and treatment of diseases and clinical conditions associated with CHST1. The function of CHST1 has been established by previous studies. The keratan sulfate proteoglycans lumican and keratocan are the major proteoglycans in the cornea and are thought to play an important role in corneal transparency. Sulfation appears to be important for the biologic function of keratan sulfate, because undersulfated keratan sulfate is synthesized in patients with macular corneal dystrophy (see OMIM Ref. No. 217800). Keratan sulfate bears sulfate groups on both N-acetylglucosamine (OMIM Ref. No. GlcNAc) and galactose residues. Fukuta et al. (1997) stated that C6ST (chondroitin 6-sulfotransferase; 603799) catalyzes sulfation of chondroitin and keratan sulfate. However, in developing cornea, keratan sulfate is actively synthesized while chondroitin 6-sulfate synthesis is minimal, suggesting that a different sulfotransferase is present in cornea with specificity towards keratan sulfate. By screening a human fetal brain library with a chick C6ST cDNA, Fukuta et al. (1997) isolated cDNAs encoding C6ST and keratan sulfate gal-6-sulfotransferase (KSGal6ST). Northern blot analysis revealed that the 2.8-kb KSGal6ST mRNA was expressed in human brain and in chick brain and cornea. A slightly larger and less abundant transcript was observed in human skeletal muscle. The predicted 411-amino acid KSGal6ST shares 37% sequence identity with chick C6ST. When the KSGal6ST cDNA was expressed in COS-7 cells, keratan sulfate sulfotransferase activity increased, but C6ST activity did not. In vitro, the partially purified KSGal6ST protein showed substrate specificity towards keratan sulfate; KSGal6ST could not utilize chondroitin as an acceptor. Fukuta et al. (1997) concluded that KSGal6ST may participate in the biosynthesis of keratan sulfate in the brain and cornea. Independently, Mazany et al. (1998) cloned genomic DNA and cDNAs corresponding to CHST1, which they called C6ST. These authors found that stable expression of the CHST1 cDNA in CHO cells increased both C6ST and keratan sulfate sulfotransferase activities. Mazany et al. (1998) suggested that the distinct pattern of CHST1 enzyme activity observed by Fukuta et al. (1997) may be due to differences between the mammalian cell lines used by the 2 groups to express the enzyme.

Full details of the abovementioned studies are described in the following publications, the disclosure of which are hereby incorporated by reference:
Iida, A.; Saito, S.; Sekine, A.; Mishima, C.; Kitamura, Y.; Kondo, K.; Harigae, S.; Osawa, S.; Nakamura, Y.: Catalog of 77 single-nucleotide polymorphisms (SNPs) in the carbohydrate sulfotransferase 1 (CHST1) and carbohydrate sulfotransferase 3 (CHST3) genes. J. Hum. Genet. 47: 14-19, 2002.; and
Mazany, K. D.; Peng, T.; Watson, C. E.; Tabas, I.; Williams, K. J.: Human chondroitin 6-sulfotransferase: cloning, gene structure, and chromosomal localization. Biochim. Biophys. Acta.

Further studies establishing the function and utilities of CHST1 are found in John Hopkins OMIM database record ID 603797, and in sited publications numbered 222-224 listed in the bibliography section hereinbelow, which are also hereby incorporated by reference. F-box and Leucine-rich Repeat Protein 7 (FBXL7, Accession NM_{—}012304) is another VGAM34 host target gene. FBXL7 BINDING SITE is HOST TARGET binding site found in the 3′ untranslated region of mRNA encoded by FBXL7, corresponding to a HOST TARGET binding site such as BINDING SITE I, BINDING SITE II or BINDING SITE III. Table 2 illustrates the complementarity of the nucleotide sequences of FBXL7 BINDING SITE, designated SEQ ID:1425, to the nucleotide sequence of VGAM34 RNA, herein designated VGAM RNA, also designated SEQ ID:369.

Another function of VGAM34 is therefore inhibition of F-box and Leucine-rich Repeat Protein 7 (FBXL7, Accession NM_{—}012304), a gene which may be involved in in phosphorylation-dependent ubiquitination. Accordingly, utilities of VGAM34 include diagnosis, prevention and treatment of diseases and clinical conditions associated with FBXL7. The function of FBXL7 has been established by previous studies. The F box, named after cyclin F (CCNF; 600227), in which it was originally observed, is an approximately 40-amino acid motif that binds SKP1 (OMIM Ref. No. 601434). F-box proteins are components of modular E3 ubiquitin protein ligases called SCFs (SKP1, OMIM Ref. No. 603134), F-box proteins), which function in phosphorylation-dependent ubiquitination. Using a yeast 2-hybrid screen with SKP1 as bait, followed by searching sequence databases, Winston et al. (1999) and Cenciarelli et al. (1999) identified 33 mammalian and 26 human F-box proteins, respectively. These contained C termini with leucine-rich repeats (FBXLs, e.g., SKP2 (OMIM Ref. No. 601436)), WD40 domains (FBXWs, e.g., BTRCP (OMIM Ref. No. 603482)), or no recognizable motifs (FBXOs, e.g., CCNF). Winston et al. (1999) predicted the presence of 12 leucine-rich repeats (LRRs) in FBXL7. RT-PCR analysis detected strong expression in all tissues tested, with highest levels in heart, kidney, liver, and lung.

Full details of the abovementioned studies are described in the following publications, the disclosure of which are hereby incorporated by reference:
Nagase, T.; Ishikawa, K.; Suyama, M.; Kikuno, R.; Hirosawa, M.; Miyajima, N.; Tanaka, A.; Kotani, H.; Nomura, N.; Ohara, O.: Prediction of the coding sequences of unidentified human genes. XII. The complete sequences of 100 new cDNA clones from brain which code for large proteins in vitro. DNA Res. 5: 355-364, 1998.; and
Winston, J. T.; Koepp, D. M.; Zhu, C.; Elledge, S. J.; Harper, J. W.: A family of mammalian F-box proteins. Curr. Biol. 9: 1180-1182, 1999.

Further studies establishing the function and utilities of FBXL7 are found in John Hopkins OMIM database record ID 605656, and in sited publications numbered 7 and 1882 listed in the bibliography section hereinbelow, which are also hereby incorporated by reference. Homeo Box C9 (HOXC9, Accession XM_{—}028620) is another VGAM34 host target gene. HOXC9 BINDING SITE is HOST TARGET binding site found in the 3′ untranslated region of mRNA encoded by HOXC9, corresponding to a HOST TARGET binding site such as BINDING SITE I, BINDING SITE II or BINDING SITE III. Table 2 illustrates the complementarity of the nucleotide sequences of HOXC9 BINDING SITE, designated SEQ ID:2592, to the nucleotide sequence of VGAM34 RNA, herein designated VGAM RNA, also designated SEQ ID:369.

Another function of VGAM34 is therefore inhibition of Homeo Box C9 (HOXC9, Accession XM_{—}028620). Accordingly, utilities of VGAM34 include diagnosis, prevention and treatment of diseases and clinical conditions associated with HOXC9. Insulin-like Growth Factor 1 (somatomedin C) (IGF1, Accession NM_{—}000618) is another VGAM34 host target gene. IGF1 BINDING SITE is HOST TARGET binding site found in the 3′ untranslated region of mRNA encoded by IGF1, corresponding to a HOST TARGET binding site such as BINDING SITE I, BINDING SITE II or BINDING SITE III. Table 2 illustrates the complementarity of the nucleotide sequences of IGF1 BINDING SITE, designated SEQ ID:763, to the nucleotide sequence of VGAM34 RNA, herein designated VGAM RNA, also designated SEQ ID:369.

Another function of VGAM34 is therefore inhibition of Insulin-like Growth Factor 1 (somatomedin C) (IGF1, Accession NM_{—}000618), a gene which are structurally and functionally related to insulin but have a much higher growth-promoting activity. Accordingly, utilities of VGAM34 include diagnosis, prevention and treatment of diseases and clinical conditions associated with IGF1. The function of IGF1 has been established by previous studies. By the solid-phase method, L1 et al. (1983) synthesized human somatomedin C, which has 70 amino acid residues and 3 disulfide bridges. Using cDNA probes in the analysis of somatic cell hybrids, Brissenden et al. (1984) and Tricoli et al. (1984) independently assigned the IGF1 structural gene to chromosome 12. Tricoli et al. (1984) regionalized the locus tentatively to 12q22-qter, where the KRAS2 (OMIM Ref. No. 190070) gene is situated. This proximity, as well as that of the HRAS1 (OMIM Ref. No. 190020) and IGF2 (OMIM Ref. No. 147470) genes on 11p and that of the NRAS (OMIM Ref. No. 164790) and NGFB (OMIM Ref. No. 162030) genes in band 1p22, suggested to Brissenden et al. (1984) that a functional or evolutionary relationship may exist between members of the RAS family of protooncogenes and growth factor genes. Chromosomal abnormalities in the region of these genes have been associated with specific forms of neoplasia. Both IGF1 and IGF2 have a striking structural homology to proinsulin. Deficiency of IGF1 was proposed as the nature of the basic defect in the African pygmy (OMIM Ref. No. 265850) and possibly also in the Laron type of dwarfism (OMIM Ref. No. 262500). The homology of chromosomes 11 and 12 is supported by the finding of yet another pair of structurally homologous loci on these 2 chromosomes. See 146000 for description of the work of Mullis et al. (1991) suggesting that the IGF1 gene may be the site of the mutation causing one form of hypochondroplasia. Using stored sera from men followed in the Baltimore Longitudinal Study on Aging, Harman et al. (2000) investigated whether the circulating IGF1 level is an independent predictor of prostate cancer and compared its predictive value with those of IGF2, IGFBP3, and prostate-specific antigen (PSA; 176820). High IGF1 and low IGF2 were independently associated with increased risk for prostate cancer, but PSA level was a much stronger predictor of prostate cancer than either IGF1 or IGF2. The absence of a relationship of IGF1 to prostate size is inconsistent with increased ascertainment in men with large prostates as the source of greater prostate cancer risk associated with IGF1. The authors concluded that IGF2 may inhibit both prostate growth and development of prostate cancer. Low birthweight is associated with later risk of type 2 diabetes and related disorders. Vaessen et al. (2002) studied the relationship between low birthweight and a polymorphism in the IGF1 gene that raises risk of type 2 diabetes and myocardial infarction. They recorded birthweight and obtained DNA for 463 adults. Individuals who did not have the wildtype allele of the polymorphism had a 215-gram lower birthweight than those homozygous for the wildtype allele. The data lent support to the hypothesis that genetic variation affecting fetal growth could account for the association between low birthweight and susceptibility to diabetes and cardiovascular disease in later life. Postnatal growth and development are coordinated by genetic and environmental influences and numerous growth factors. Le Roith et al. (2001) reviewed the essential role that the GH-IGFI axis plays in these processes. Although the GH-IGFI axis is a closely coordinated system, both GH and IGFI have independent actions, many of which have become apparent more recently following the characterization of clinical syndromes and the development of mouse models. Genetic manipulation of mice has enabled investigators to reexamine many of the established hypotheses regarding the GH-IGFI axis. Results gleaned from a mouse model created by tissue-specific gene deletion of liver IGFI (Yakar et. al, 1999; Sjogren et al., 1999) enabled investigators to reevaluate the original ‘somatomedin hypothesis.’ Animal model experiments lend further support to the function of IGF1. Aging skeletal muscles suffer a steady decline in mass and functional performance, and compromised muscle integrity as fibrotic invasions replace contractile tissue. The same programmed deficits in muscle structure and function are found in numerous neurodegenerative syndromes and disease-related cachexia. Musaro et al. (2001) generated a model of persistent, functional myocyte hypertrophy using a tissue-restricted transgene encoding a locally acting isoform of Igf1 that is expressed in skeletal muscle. Transgenic embryos developed normally, and postnatal increases in muscle mass and strength were not accompanied by the additional pathologic changes seen in other Igf1 transgenic models. Expression of Gata2 (OMIM Ref. No. 137295), a transcription factor normally undetected in skeletal muscle, marked hypertrophic myocytes that escaped age-related muscle atrophy and retained the proliferative response to muscle injury characteristic of younger animals. The observations were thought to suggest usefulness of localized expression of this transgene as a clinical strategy for the treatment of age- or disease-related muscle frailty.

It is appreciated that the abovementioned animal model for IGF1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Musaro, A.; McCullagh, K.; Paul, A.; Houghton, L.; Dobrowolny, G.; Molinaro, M.; Barton, E. R.; Sweeney, H. L.; Rosenthal, N.: Localized Igf-1 transgene expression sustains hypertrophy and regeneration in senescent skeletal muscle. Nature Genet. 27: 195-200, 2001.; and
Le Roith, D.; Scavo, L.; Butler, A.: What is the role of circulating IGF-I? Trends Endocr. Metab. 12: 48-52, 2001.

Further studies establishing the function and utilities of IGF1 are found in John Hopkins OMIM database record ID 147440, and in sited publications numbered 2670-2672, 1047-1048, 2673-2675, 2688, 2694, 2698-2696, 695, 2697, 2699-2700, 329, 2701-2703, 1049, 2704-2708, 2582, 2709-2716, 858-860, and 885-889 listed in the bibliography section hereinbelow, which are also hereby incorporated by reference. Integrin, Alpha L (antigen CD11A (p180), Lymphocyte Function-associated Antigen 1; Alpha Polypeptide) (ITGAL, Accession NM_{—}002209) is another VGAM34 host target gene. ITGAL BINDING SITE is HOST TARGET binding site found in the 3′ untranslated region of mRNA encoded by ITGAL, corresponding to a HOST TARGET binding site such as BINDING SITE I, BINDING SITE II or BINDING SITE III. Table 2 illustrates the complementarity of the nucleotide sequences of ITGAL BINDING SITE, designated SEQ ID:907, to the nucleotide sequence of VGAM34 RNA, herein designated VGAM RNA, also designated SEQ ID:369.

Another function of VGAM34 is therefore inhibition of Integrin, Alpha L (antigen CD11A (p180), Lymphocyte Function-associated Antigen 1; Alpha Polypeptide) (ITGAL, Accession NM_{—}002209), a gene which is a receptor for icam1, icam2, icam3 and icam4. it is involved in a variety of immune phenomena including leukocyte-endothelial cell interaction, cytotoxic t-cell mediated killing, and antibody dependent killing by granulocytes and monocytes. Accordingly, utilities of VGAM34 include diagnosis, prevention and treatment of diseases and clinical conditions associated with ITGAL. The function of ITGAL has been established by previous studies. See 120980 and 151510. Lymphocyte function-associated antigen-1 (LFA-1) shares a beta subunit (see OMIM Ref. No. 116920) with other members of a family of leukocyte surface membrane antigens but has a unique alpha subunit (Sanchez-Madrid et al., 1983). LFA-1 is expressed on lymphocytes and phagocytic cells. The LFA-1 molecule is involved in the adhesion of cytotoxic T cells to their target cells. Patients with LFA-1 immunodeficiency disease (see OMIM Ref. No. 116920) have recurrent life-threatening infections, show deficiency of the beta chain of all 3 molecules, LFA-1, Mac-1 (macrophage antigen-1), and p150,95, and display profound defects in adhesion-dependent granulocyte, monocyte, and B- and T-lymphocyte functions. The alpha subunits were designated by Marlin et al. (1986) as alpha-L for LFA-1, alpha-M for Mac-1, and alpha-X for p150,95. Lu and Cyster (2002) studied the mechanisms that control localization of marginal zone B cells. They demonstrated that marginal zone B cells express elevated levels of the integrins LFA-1 and alpha-4-beta-1 (see OMIM Ref. No. 192975 and 135630) and that the marginal zone B cells bind to the ligands ICAM1 (OMIM Ref. No. 147840) and VCAM1 (OMIM Ref. No. 192225). These ligands are expressed within the marginal zone in a lymphotoxin-dependent manner. Combined inhibition of LFA-1 and alpha-4-beta-1 causes a rapid and selective release of B cells from the marginal zone. Furthermore, lipopolysaccharide-triggered marginal zone B cell relocalization involves downregulation of integrin-mediated adhesion. Lu and Cyster (2002) concluded that their studies identified key requirements for marginal zone B cell localization and established a role for integrins in peripheral lymphoid tissue compartmentalization

Full details of the abovementioned studies are described in the following publications, the disclosure of which are hereby incorporated by reference:
Lu, T. T.; Cyster, J. G.: Integrin-mediated long-term B cell retention in the splenic marginal zone. Science 297: 409-412, 2002.; and
Marlin, S. D.; Morton, C. C.; Anderson, D. C.; Springer, T. A.: LFA-1 immunodeficiency disease: definition of the genetic defect and chromosomal mapping of alpha and beta subunits of t.

Further studies establishing the function and utilities of ITGAL are found in John Hopkins OMIM database record ID 153370, and in sited publications numbered 1120-1121, 684, 77 and 835-836 listed in the bibliography section hereinbelow, which are also hereby incorporated by reference. Lamin B Receptor (LBR, Accession XM_{—}001795) is another VGAM34 host target gene. LBR BINDING SITE is HOST TARGET binding site found in the 3′ untranslated region of mRNA encoded by LBR, corresponding to a HOST TARGET binding site such as BINDING SITE I, BINDING SITE II or BINDING SITE III. Table 2 illustrates the complementarity of the nucleotide sequences of LBR BINDING SITE, designated SEQ ID:2522, to the nucleotide sequence of VGAM34 RNA, herein designated VGAM RNA, also designated SEQ ID:369.

Another function of VGAM34 is therefore inhibition of Lamin B Receptor (LBR, Accession XM_{—}001795). Accordingly, utilities of VGAM34 include diagnosis, prevention and treatment of diseases and clinical conditions associated with LBR. Major Intrinsic Protein of Lens Fiber (MIP, Accession NM_{—}012064) is another VGAM34 host target gene. MIP BINDING SITE is HOST TARGET binding site found in the 3′ untranslated region of mRNA encoded by MIP, corresponding to a HOST TARGET binding site such as BINDING SITE I, BINDING SITE II or BINDING SITE III. Table 2 illustrates the complementarity of the nucleotide sequences of MIP BINDING SITE, designated SEQ ID:1399, to the nucleotide sequence of VGAM34 RNA, herein designated VGAM RNA, also designated SEQ ID:369.

Another function of VGAM34 is therefore inhibition of Major Intrinsic Protein of Lens Fiber (MIP, Accession NM_{—}012064). Accordingly, utilities of VGAM34 include diagnosis, prevention and treatment of diseases and clinical conditions associated with MIP. Relaxin 2 (H2) (RLN2, Accession NM_{—}005059) is another VGAM34 host target gene. RLN2 BINDING SITE is HOST TARGET binding site found in the 3′ untranslated region of mRNA encoded by RLN2, corresponding to a HOST TARGET binding site such as BINDING SITE I, BINDING SITE II or BINDING SITE III. Table 2 illustrates the complementarity of the nucleotide sequences of RLN2 BINDING SITE, designated SEQ ID:1175, to the nucleotide sequence of VGAM34 RNA, herein designated VGAM RNA, also designated SEQ ID:369.

Another function of VGAM34 is therefore inhibition of Relaxin 2 (H2) (RLN2, Accession NM_{—}005059). Accordingly, utilities of VGAM34 include diagnosis, prevention and treatment of diseases and clinical conditions associated with RLN2. SART2 (Accession NM_{—}013352) is another VGAM34 host target gene. SART2 BINDING SITE is HOST TARGET binding site found in the 3′ untranslated region of mRNA encoded by SART2, corresponding to a HOST TARGET binding site such as BINDING SITE I, BINDING SITE II or BINDING SITE III. Table 2 illustrates the complementarity of the nucleotide sequences of SART2 BINDING SITE, designated SEQ ID: 1444, to the nucleotide sequence of VGAM34 RNA, herein designated VGAM RNA, also designated SEQ ID:369.

Another function of VGAM34 is therefore inhibition of SART2 (Accession NM_{—}013352). Accordingly, utilities of VGAM34 include diagnosis, prevention and treatment of diseases and clinical conditions associated with SART2. Thiamin Pyrophosphokinase 1 (TPK1, Accession NM_{—}022445) is another VGAM34 host target gene. TPK1 BINDING SITE is HOST TARGET binding site found in the 3′ untranslated region of mRNA encoded by TPK1, corresponding to a HOST TARGET binding site such as BINDING SITE I, BINDING SITE II or BINDING SITE III. Table 2 illustrates the complementarity of the nucleotide sequences of TPK1 BINDING SITE, designated SEQ ID:1987, to the nucleotide sequence of VGAM34 RNA, herein designated VGAM RNA, also designated SEQ ID:369.

Another function of VGAM34 is therefore inhibition of Thiamin Pyrophosphokinase 1 (TPK1, Accession NM_{—}022445), a gene which catalyzes the conversion of thiamine, a form of vitamin B1, to thiamine pyrophosphate. Accordingly, utilities of VGAM34 include diagnosis, prevention and treatment of diseases and clinical conditions associated with TPK1. The function of TPK1 has been established by previous studies. By Northern blot analysis, Nosaka et al. (1999) detected expression of mouse Tpk1 predominantly in kidney and liver, with very faint expression in heart, brain, and testis. In contrast to the tissue-specific expression of mouse Tpk1, Nosaka et al. (2001) reported broad expression of a human 2.5-kb TPK1 transcript. They detected very low expression in a variety of human tissues and relatively abundant expression in heart, kidney, and peripheral leukocytes. By Northern blot analysis, Zhao et al. (2001) detected broad expression of a 2.6-kb TPK1 transcript, with highest levels in testis and in those tissues involved in thiamine absorption (small intestine) and reabsorption (OMIM Ref. No. kidney). They also detected a smaller (1-1.5 kb), testis-specific TPK1 transcript. From results of cell culture experiments, Nosaka et al. (1999) and Nosaka et al. (2001) concluded that thiamine or a thiamine derivative does not participate in the regulation of TPK1. Nosaka et al. (2001) detected no difference in TPK1 expression in cultured fibroblasts from normal subjects or from patients with thiamine-responsive megaloblastic anemia (OMIM Ref. No. 249270).

Full details of the abovementioned studies are described in the following publications, the disclosure of which are hereby incorporated by reference:
Zhao, R.; Gao, F.; Goldman, I. D.: Molecular cloning of human thiamin pyrophosphokinase. Biochim. Biophys. Acta 1517: 320-322, 2001.; and
Nosaka, K.; Onozuka, M.; Nishino, H.; Nishimura, H.; Kawasaki, Y.; Ueyama, H.: Molecular cloning and expression of a mouse thiamin pyrophosphokinase cDNA. J. Biol. Chem. 274: 34129-3413.

Further studies establishing the function and utilities of TPK1 are found in John Hopkins OMIM database record ID 606370, and in sited publications numbered 1437-1439 listed in the bibliography section hereinbelow, which are also hereby incorporated by reference. Thyroid Hormone Receptor Interactor 12 (TRIP12, Accession NM_{—}004238) is another VGAM34 host target gene. TRIP12 BINDING SITE is HOST TARGET binding site found in the 3′ untranslated region of mRNA encoded by TRIP12, corresponding to a HOST TARGET binding site such as BINDING SITE I, BINDING SITE II or BINDING SITE III. Table 2 illustrates the complementarity of the nucleotide sequences of TRIP12 BINDING SITE, designated SEQ ID: 1094, to the nucleotide sequence of VGAM34 RNA, herein designated VGAM RNA, also designated SEQ ID:369.

Another function of VGAM34 is therefore inhibition of Thyroid Hormone Receptor Interactor 12 (TRIP12, Accession NM_{—}004238), a gene which interacts with the ligand binding domain of the thyroid hormone receptor (in a thyroid hormone t3-independent manner) and with retinoid×receptor (r×r). Accordingly, utilities of VGAM34 include diagnosis, prevention and treatment of diseases and clinical conditions associated with TRIP12. The function of TRIP12 has been established by previous studies. By sequencing cDNAs randomly selected from a cDNA library derived from a human immature myeloid cell line, Nomura et al. (1994) isolated a cDNA encoding TRIP12, which they called KIAA0045. The predicted 1,992-amino acid TRIP12 protein contains a putative transmembrane domain. TRIP12 shares amino acid sequence similarity with the S. cerevisiae YKL010C gene product in 3 regions, with percent identities ranging from approximately 33 to 43%. Northern blot analysis detected TRIP12 expression in all 16 human tissues examined, with the highest expression in skeletal muscle and testis, lower expression in heart, kidney, spleen, thymus, prostate, ovary, placenta, and peripheral blood leukocytes, and the lowest expression in brain, lung, liver, pancreas, small intestine, and colon. The thyroid hormone receptors (TRs) are hormone-dependent transcription factors that regulate expression of a variety of specific target genes. They must specifically interact with a number of proteins as they progress from their initial translation and nuclear translocation to heterodimerization with retinoid X receptors (RXRs), functional interactions with other transcription factors and the basic transcriptional apparatus, and eventually, degradation. To help elucidate the mechanisms that underlie the transcriptional effects and other potential functions of TRs, Lee et al. (1995) used the yeast interaction trap, a version of the yeast 2-hybrid system, to identify proteins that specifically interact with the ligand-binding domain of rat TR-beta (THRB; 190160). They isolated HeLa cell cDNAs encoding several different TR-interacting proteins (TRIPs), including TRIP12. TRIP12 interacted with rat Thrb only in the absence of thyroid hormone. It did not interact with RXR-alpha (RXRA; 180245) or the glucocorticoid receptor (NR3C1; 138040) under any condition. TRIP12 shares sequence similarity with E6AP (UBE3A; 601623), a ubiquitin-protein ligase.

Full details of the abovementioned studies are described in the following publications, the disclosure of which are hereby incorporated by reference:
Lee, J. W.; Choi, H.-S.; Gyuris, J.; Brent, R.; Moore, D. D.; Two classes of proteins dependent on either the presence or absence of thyroid hormone for interaction with the thyroid hormone receptor. Molec. Endocr. 9: 243-254, 1995.; and
Nomura, N.; Nagase, T.; Miyajima, N.; Sazuka, T.; Tanaka, A.; Sato, S.; Seki, N.; Kawarabayasi, Y.; Ishikawa, K.; Tabata, S.: Prediction of the coding sequences of unidentified human g.

Further studies establishing the function and utilities of TRIP12 are found in John Hopkins OMIM database record ID 604506, and in sited publications numbered 1444 listed in the bibliography section hereinbelow, which are also hereby incorporated by reference. Bromodomain Containing 3 (BRD3, Accession NM_{—}007371) is another VGAM34 host target gene. BRD3 BINDING SITE is HOST TARGET binding site found in the 3′ untranslated region of mRNA encoded by BRD3, corresponding to a HOST TARGET binding site such as BINDING SITE I, BINDING SITE II or BINDING SITE III. Table 2 illustrates the complementarity of the nucleotide sequences of BRD3 BINDING SITE, designated SEQ ID:1398, to the nucleotide sequence of VGAM34 RNA, herein designated VGAM RNA, also designated SEQ ID:369.

Another function of VGAM34 is therefore inhibition of Bromodomain Containing 3 (BRD3, Accession NM_{—}007371). Accordingly, utilities of VGAM34 include diagnosis, prevention and treatment of diseases and clinical conditions associated with BRD3. FLJ13397 (Accession NM_{—}024948) is another VGAM34 host target gene. FLJ13397 BINDING SITE is HOST TARGET binding site found in the 3′ untranslated region of mRNA encoded by FLJ13397, corresponding to a HOST TARGET binding site such as BINDING SITE I, BINDING SITE II or BINDING SITE III. Table 2 illustrates the complementarity of the nucleotide sequences of FLJ13397 BINDING SITE, designated SEQ ID:2116, to the nucleotide sequence of VGAM34 RNA, herein designated VGAM RNA, also designated SEQ ID:369.

Another function of VGAM34 is therefore inhibition of FLJ13397 (Accession NM_{—}024948). Accordingly, utilities of VGAM34 include diagnosis, prevention and treatment of diseases and clinical conditions associated with FLJ13397. FLJ22202 (Accession NM_{—}024883) is another VGAM34 host target gene. FLJ22202 BINDING SITE is HOST TARGET binding site found in the 3′ untranslated region of mRNA encoded by FLJ22202, corresponding to a HOST TARGET binding site such as BINDING SITE I, BINDING SITE II or BINDING SITE III. Table 2 illustrates the complementarity of the nucleotide sequences of FLJ22202 BINDING SITE, designated SEQ ID:2103, to the nucleotide sequence of VGAM34 RNA, herein designated VGAM RNA, also designated SEQ ID:369.

Another function of VGAM34 is therefore inhibition of FLJ22202 (Accession NM_{—}024883). Accordingly, utilities of VGAM34 include diagnosis, prevention and treatment of diseases and clinical conditions associated with FLJ22202. GROS1 (Accession NM_{—}022356) is another VGAM34 host target gene. GROS1 BINDING SITE is HOST TARGET binding site found in the 3′ untranslated region of mRNA encoded by GROS1, corresponding to a HOST TARGET binding site such as BINDING SITE I, BINDING SITE II or BINDING SITE III. Table 2 illustrates the complementarity of the nucleotide sequences of GROS1 BINDING SITE, designated SEQ ID:1986, to the nucleotide sequence of VGAM34 RNA, herein designated VGAM RNA, also designated SEQ ID:369.

Another function of VGAM34 is therefore inhibition of GROS1 (Accession NM_{—}022356). Accordingly, utilities of VGAM34 include diagnosis, prevention and treatment of diseases and clinical conditions associated with GROS1. KIAA0981 (Accession XM_{—}028867) is another VGAM34 host target gene. KIAA0981 BINDING SITE is HOST TARGET binding site found in the 3′ untranslated region of mRNA encoded by KIAA0981, corresponding to a HOST TARGET binding site such as BINDING SITE I, BINDING SITE II or BINDING SITE III. Table 2 illustrates the complementarity of the nucleotide sequences of KIAA0981 BINDING SITE, designated SEQ ID:2598, to the nucleotide sequence of VGAM34 RNA, herein designated VGAM RNA, also designated SEQ ID:369.

Another function of VGAM34 is therefore inhibition of KIAA0981 (Accession XM_{—}028867). Accordingly, utilities of VGAM34 include diagnosis, prevention and treatment of diseases and clinical conditions associated with KIAA0981. KIAA1243 (Accession XM_{—}057057) is another VGAM34 host target gene. KIAA1243 BINDING SITE is HOST TARGET binding site found in the 3′ untranslated region of mRNA encoded by KIAA1243, corresponding to a HOST TARGET binding site such as BINDING SITE I, BINDING SITE II or BINDING SITE III. Table 2 illustrates the complementarity of the nucleotide sequences of KIAA1243 BINDING SITE, designated SEQ ID:2978, to the nucleotide sequence of VGAM34 RNA, herein designated VGAM RNA, also designated SEQ ID:369.

Another function of VGAM34 is therefore inhibition of KIAA1243 (Accession XM_{—}057057). Accordingly, utilities of VGAM34 include diagnosis, prevention and treatment of diseases and clinical conditions associated with KIAA1243. KIAA1462 (Accession XM_{—}166132) is another VGAM34 host target gene. KIAA1462 BINDING SITE is HOST TARGET binding site found in the 3′ untranslated region of mRNA encoded by KIAA1462, corresponding to a HOST TARGET binding site such as BINDING SITE I, BINDING SITE II or BINDING SITE III. Table 2 illustrates the complementarity of the nucleotide sequences of KIAA1462 BINDING SITE, designated SEQ ID:3524, to the nucleotide sequence of VGAM34 RNA, herein designated VGAM RNA, also designated SEQ ID:369.

Another function of VGAM34 is therefore inhibition of KIAA1462 (Accession XM_{—}166132). Accordingly, utilities of VGAM34 include diagnosis, prevention and treatment of diseases and clinical conditions associated with KIAA1462. MGC1136 (Accession NM_{—}024025) is another VGAM34 host target gene. MGC1136 BINDING SITE is HOST TARGET binding site found in the 3′ untranslated region of mRNA encoded by MGC1136, corresponding to a HOST TARGET binding site such as BINDING SITE I, BINDING SITE II or BINDING SITE III. Table 2 illustrates the complementarity of the nucleotide sequences of MGC1136 BINDING SITE, designated SEQ ID:2041, to the nucleotide sequence of VGAM34 RNA, herein designated VGAM RNA, also designated SEQ ID:369.

Another function of VGAM34 is therefore inhibition of MGC1136 (Accession NM_{—}024025). Accordingly, utilities of VGAM34 include diagnosis, prevention and treatment of diseases and clinical conditions associated with MGC1136. MSTP043 (Accession NM_{—}031953) is another VGAM34 host target gene. MSTP043 BINDING SITE is HOST TARGET binding site found in the 3′ untranslated region of mRNA encoded by MSTP043, corresponding to a HOST TARGET binding site such as BINDING SITE I, BINDING SITE II or BINDING SITE III. Table 2 illustrates the complementarity of the nucleotide sequences of MSTP043 BINDING SITE, designated SEQ ID:2223, to the nucleotide sequence of VGAM34 RNA, herein designated VGAM RNA, also designated SEQ ID:369.

Another function of VGAM34 is therefore inhibition of MSTP043 (Accession NM_{—}031953). Accordingly, utilities of VGAM34 include diagnosis, prevention and treatment of diseases and clinical conditions associated with MSTP043. Nup43 (Accession NM_{—}024647) is another VGAM34 host target gene. Nup43 BINDING SITE is HOST TARGET binding site found in the 3′ untranslated region of mRNA encoded by Nup43, corresponding to a HOST TARGET binding site such as BINDING SITE I, BINDING SITE II or BINDING SITE III. Table 2 illustrates the complementarity of the nucleotide sequences of Nup43 BINDING SITE, designated SEQ ID:2075, to the nucleotide sequence of VGAM34 RNA, herein designated VGAM RNA, also designated SEQ ID:369.

Another function of VGAM34 is therefore inhibition of Nup43 (Accession NM_{—}024647). Accordingly, utilities of VGAM34 include diagnosis, prevention and treatment of diseases and clinical conditions associated with Nup43. Ribosomal Protein S6 Kinase, 90 kDa, Polypeptide 4 (RPS6KA4, Accession NM_{—}003942) is another VGAM34 host target gene. RPS6KA4 BINDING SITE is HOST TARGET binding site found in the 3′ untranslated region of mRNA encoded by RPS6KA4, corresponding to a HOST TARGET binding site such as BINDING SITE I, BINDING SITE II or BINDING SITE III. Table 2 illustrates the complementarity of the nucleotide sequences of RPS6KA4 BINDING SITE, designated SEQ ID: 1072, to the nucleotide sequence of VGAM34 RNA, herein designated VGAM RNA, also designated SEQ ID:369.

Another function of VGAM34 is therefore inhibition of Ribosomal Protein S6 Kinase, 90 kDa, Polypeptide 4 (RPS6KA4, Accession NM_{—}003942). Accordingly, utilities of VGAM34 include diagnosis, prevention and treatment of diseases and clinical conditions associated with RPS6KA4. Solute Carrier Family 17 (sodium-dependent inorganic phosphate cotransporter), Member 6 (SLC17A6, Accession NM_{—}020346) is another VGAM34 host target gene. SLC17A6 BINDING SITE is HOST TARGET binding site found in the 3′ untranslated region of mRNA encoded by SLC17A6, corresponding to a HOST TARGET binding site such as BINDING SITE I, BINDING SITE II or BINDING SITE III. Table 2 illustrates the complementarity of the nucleotide sequences of SLC17A6 BINDING SITE, designated SEQ ID:1911, to the nucleotide sequence of VGAM34 RNA, herein designated VGAM RNA, also designated SEQ ID:369.

Another function of VGAM34 is therefore inhibition of Solute Carrier Family 17 (sodium-dependent inorganic phosphate cotransporter), Member 6 (SLC17A6, Accession NM_{—}020346). Accordingly, utilities of VGAM34 include diagnosis, prevention and treatment of diseases and clinical conditions associated with SLC17A6. TERA (Accession NM_{—}021238) is another VGAM34 host target gene. TERA BINDING SITE is HOST TARGET binding site found in the 3′ untranslated region of mRNA encoded by TERA, corresponding to a HOST TARGET binding site such as BINDING SITE I, BINDING SITE II or BINDING SITE III. Table 2 illustrates the complementarity of the nucleotide sequences of TERA BINDING SITE, designated SEQ ID:1949, to the nucleotide sequence of VGAM34 RNA, herein designated VGAM RNA, also designated SEQ ID:369.

Another function of VGAM34 is therefore inhibition of TERA (Accession NM_{—}021238). Accordingly, utilities of VGAM34 include diagnosis, prevention and treatment of diseases and clinical conditions associated with TERA. Vav 3 Oncogene (VAV3, Accession NM_{—}006113) is another VGAM34 host target gene. VAV3 BINDING SITE is HOST TARGET binding site found in the 3′ untranslated region of mRNA encoded by VAV3, corresponding to a HOST TARGET binding site such as BINDING SITE I, BINDING SITE II or BINDING SITE III. Table 2 illustrates the complementarity of the nucleotide sequences of VAV3 BINDING SITE, designated SEQ ID:1273, to the nucleotide sequence of VGAM34 RNA, herein designated VGAM RNA, also designated SEQ ID:369.

Another function of VGAM34 is therefore inhibition of Vav 3 Oncogene (VAV3, Accession NM_{—}006113). Accordingly, utilities of VGAM34 include diagnosis, prevention and treatment of diseases and clinical conditions associated with VAV3. LOC120892 (Accession XM_{—}058513) is another VGAM34 host target gene. LOC120892 BINDING SITE is HOST TARGET binding site found in the 3′ untranslated region of mRNA encoded by LOC120892, corresponding to a HOST TARGET binding site such as BINDING SITE I, BINDING SITE II or BINDING SITE III. Table 2 illustrates the complementarity of the nucleotide sequences of LOC120892 BINDING SITE, designated SEQ ID:2987, to the nucleotide sequence of VGAM34 RNA, herein designated VGAM RNA, also designated SEQ ID:369.

Another function of VGAM34 is therefore inhibition of LOC120892 (Accession XM_{—}058513). Accordingly, utilities of VGAM34 include diagnosis, prevention and treatment of diseases and clinical conditions associated with LOC120892. LOC155434 (Accession XM_{—}098723) is another VGAM34 host target gene. LOC155434 BINDING SITE is HOST TARGET binding site found in the 3′ untranslated region of mRNA encoded by LOC155434, corresponding to a HOST TARGET binding site such as BINDING SITE I, BINDING SITE II or BINDING SITE III. Table 2 illustrates the complementarity of the nucleotide sequences of LOC155434 BINDING SITE, designated SEQ ID:3376, to the nucleotide sequence of VGAM34 RNA, herein designated VGAM RNA, also designated SEQ ID:369.

Another function of VGAM34 is therefore inhibition of LOC155434 (Accession XM_{—}098723). Accordingly, utilities of VGAM34 include diagnosis, prevention and treatment of diseases and clinical conditions associated with LOC155434. LOC169966 (Accession XM_{—}093010) is another VGAM34 host target gene. LOC169966 BINDING SITE is HOST TARGET binding site found in the 3′ untranslated region of mRNA encoded by LOC169966, corresponding to a HOST TARGET binding site such as BINDING SITE I, BINDING SITE II or BINDING SITE III. Table 2 illustrates the complementarity of the nucleotide sequences of LOC169966 BINDING SITE, designated SEQ ID:3252, to the nucleotide sequence of VGAM34 RNA, herein designated VGAM RNA, also designated SEQ ID:369.

Another function of VGAM34 is therefore inhibition of LOC169966 (Accession XM_{—}093010). Accordingly, utilities of VGAM34 include diagnosis, prevention and treatment of diseases and clinical conditions associated with LOC169966. LOC221272 (Accession XM_{—}168050) is another VGAM34 host target gene. LOC221272 BINDING SITE is HOST TARGET binding site found in the 3′ untranslated region of mRNA encoded by LOC221272, corresponding to a HOST TARGET binding site such as BINDING SITE I, BINDING SITE II or BINDING SITE III. Table 2 illustrates the complementarity of the nucleotide sequences of LOC221272 BINDING SITE, designated SEQ ID:3616, to the nucleotide sequence of VGAM34 RNA, herein designated VGAM RNA, also designated SEQ ID:369.

Another function of VGAM34 is therefore inhibition of LOC221272 (Accession XM_{—}168050). Accordingly, utilities of VGAM34 include diagnosis, prevention and treatment of diseases and clinical conditions associated with LOC221272. LOC221968 (Accession XM_{—}166524) is another VGAM34 host target gene. LOC221968 BINDING SITE is HOST TARGET binding site found in the 3′ untranslated region of mRNA encoded by LOC221968, corresponding to a HOST TARGET binding site such as BINDING SITE I, BINDING SITE II or BINDING SITE III. Table 2 illustrates the complementarity of the nucleotide sequences of LOC221968 BINDING SITE, designated SEQ ID:3569, to the nucleotide sequence of VGAM34 RNA, herein designated VGAM RNA, also designated SEQ ID:369.

Another function of VGAM34 is therefore inhibition of LOC221968 (Accession XM_{—}166524). Accordingly, utilities of VGAM34 include diagnosis, prevention and treatment of diseases and clinical conditions associated with LOC221968. LOC86651 (Accession XM_{—}044052) is another VGAM34 host target gene. LOC86651 BINDING SITE is HOST TARGET binding site found in the 5′ untranslated region of mRNA encoded by LOC86651, corresponding to a HOST TARGET binding site such as BINDING SITE I, BINDING SITE II or BINDING SITE III. Table 2 illustrates the complementarity of the nucleotide sequences of LOC86651 BINDING SITE, designated SEQ ID:2830, to the nucleotide sequence of VGAM34 RNA, herein designated VGAM RNA, also designated SEQ ID:369.

Another function of VGAM34 is therefore inhibition of LOC86651 (Accession XM_{—}044052). Accordingly, utilities of VGAM34 include diagnosis, prevention and treatment of diseases and clinical conditions associated with LOC86651. LOC90322 (Accession XM_{—}030903) is another VGAM34 host target gene. LOC90322 BINDING SITE is HOST TARGET binding site found in the 3′ untranslated region of mRNA encoded by LOC90322, corresponding to a HOST TARGET binding site such as BINDING SITE I, BINDING SITE II or BINDING SITE III. Table 2 illustrates the complementarity of the nucleotide sequences of LOC90322 BINDING SITE, designated SEQ ID:2632, to the nucleotide sequence of VGAM34 RNA, herein designated VGAM RNA, also designated SEQ ID:369.

Another function of VGAM34 is therefore inhibition of LOC90322 (Accession XM_{—}030903). Accordingly, utilities of VGAM34 include diagnosis, prevention and treatment of diseases and clinical conditions associated with LOC90322. FIG. 1 further provides a conceptual description of a novel bioinformatically detected viral gene of the present invention, referred to here as Viral Genomic Address Messenger 35 (VGAM35) viral gene, which modulates expression of respective host target genes thereof, the function and utility of which host target genes is known in the art.

VGAM35 is a novel bioinformatically detected regulatory, non protein coding, viral micro RNA (miRNA) gene. The method by which VGAM35 was detected is described hereinabove with reference to FIGS. 1-8.

VGAM35 gene, herein designated VGAM GENE, is a viral gene contained in the genome of Vaccinia Virus. VGAM35 host target gene, herein designated VGAM HOST TARGET GENE, is a human gene contained in the human genome.

VGAM35 gene encodes a VGAM35 precursor RNA, herein designated VGAM PRECURSOR RNA. Similar to other miRNA genes, and unlike most ordinary genes, VGAM35 precursor RNA does not encode a protein. A nucleotide sequence identical or highly similar to the nucleotide sequence of VGAM35 precursor RNA is designated SEQ ID:21, and is provided hereinbelow with reference to the sequence listing part. Nucleotide sequence SEQ ID:21 is located at position 642 relative to the genome of Vaccinia Virus.

VGAM35 precursor RNA folds onto itself, forming VGAM35 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM35 folded precursor RNA into VGAM35 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM35 RNA is designated SEQ ID:370, and is provided hereinbelow with reference to the sequence listing part.

VGAM35 host target gene, herein designated VGAM HOST TARGET GENE, encodes a corresponding messenger RNA, VGAM35 host target RNA, herein designated VGAM HOST TARGET RNA. VGAM35 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM35 RNA, herein designated VGAM RNA, binds complementarily to one or more host target binding sites located in untranslated regions of VGAM35 host target RNA, herein designated VGAM HOST TARGET RNA. This complementary binding is due to the fact that the nucleotide sequence of VGAM35 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM35 RNA, herein designated VGAM RNA, may have a different number of host target binding sites in untranslated regions of a VGAM35 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM35 RNA, herein designated VGAM RNA, to host target binding sites on VGAM35 host target RNA, herein designated VGAM HOST TARGET RNA, such as BINDING SITE I, BINDING SITE II and BINDING SITE III, inhibits translation of VGAM35 host target RNA into VGAM35 host target protein, herein designated VGAM HOST TARGET PROTEIN. VGAM host target protein is therefore outlined by a broken line.

It is appreciated that VGAM35 host target gene, herein designated VGAM HOST TARGET GENE, in fact represents a plurality of VGAM35 host target genes. The mRNA of each one of this plurality of VGAM35 host target genes comprises one or more host target binding sites, each having a nucleotide sequence which is at least partly complementary to VGAM35 RNA, herein designated VGAM RNA, and which when bound by VGAM35 RNA causes inhibition of translation of respective one or more VGAM35 host target proteins.

It is further appreciated by one skilled in the art that the mode of translational inhibition illustrated by FIG. 1 with specific reference to translational inhibition exerted by VGAM35 gene, herein designated VGAM GENE, on one or more VGAM35 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM35 is inhibition of expression of host target genes, as part of a novel viral mechanism of attacking a host. Accordingly, utilities of VGAM35 include diagnosis, prevention and treatment of viral infection by Vaccinia Virus. Specific functions, and accordingly utilities, of VGAM35 correlate with, and may be deduced from, the identity of the host target genes which VGAM35 binds and inhibits, and the function of these host target genes, as elaborated hereinbelow.

Nucleotide sequences of the VGAM35 precursor RNA, herein designated VGAM PRECURSOR RNA, and of the ‘diced’ VGAM35 RNA, herein designated VGAM RNA, and a schematic representation of the secondary folding of VGAM35 folded precursor RNA, herein designated VGAM FOLDED PRECURSOR RNA, of VGAM35 are further described hereinbelow with reference to Table 1.

Nucleotide sequences of host target binding sites, such as BINDING SITE-I, BINDING SITE-II and BINDING SITE-III of FIG. 1, found on VGAM35 host target RNA, and schematic representation of the complementarity of each of these host target binding sites to VGAM35 RNA, herein designated VGAM RNA, are described hereinbelow with reference to Table 2.

As mentioned hereinabove with reference to FIG. 1, a function of VGAM35 gene, herein designated VGAM is inhibition of expression of VGAM35 target genes. It is appreciated that specific functions, and accordingly utilities, of VGAM35 correlate with, and may be deduced from, the identity of the target genes which VGAM35 binds and inhibits, and the function of these target genes, as elaborated hereinbelow.

Myosin IE (MYO1E, Accession NM_{—}004998) is a VGAM35 host target gene. MYO1E BINDING SITE is HOST TARGET binding site found in the 5′ untranslated region of mRNA encoded by MYO1E, corresponding to a HOST TARGET binding site such as BINDING SITE I, BINDING SITE II or BINDING SITE III. Table 2 illustrates the complementarity of the nucleotide sequences of MYO1E BINDING SITE, designated SEQ ID:1168, to the nucleotide sequence of VGAM35 RNA, herein designated VGAM RNA, also designated SEQ ID:370.

A function of VGAM35 is therefore inhibition of Myosin IE (MYO1E, Accession NM_{—}004998), a gene which is an unconventional myosin. Accordingly, utilities of VGAM35 include diagnosis, prevention and treatment of diseases and clinical conditions associated with MYO1E. The function of MYO1E has been established by previous studies. Bement et al. (1994) cloned a human unconventional myosin gene, MYO1E, encoding a predicted 127-kD polypeptide of 1,109 amino acids. The gene, which they designated myosin IC, contains a characteristic N-terminal myosin head, a single ‘IQ motif’ predicted to bind a single myosin light chain, and a C-terminal tail with a putative membrane-binding site. They also noted the presence of a C-terminal src-homology domain, reminiscent of ‘long-tailed’ myosins I from amoeboid organisms. By Northern analysis, Bement et al. (1994) detected ubiquitous expression of MYO1E. Hasson et al. (1996) used fluorescence in situ hybridization to map the loci for 4 unconventional myosin loci in humans: MYO1E (formerly MYO1C), MYO1A (OMIM Ref. No. 601478), MYO1F (OMIM Ref. No. 601480), and MYO10 (OMIM Ref. No. 601481). The MYO1E gene was found to be located on 15q21-q22 in the precise location predicted from its location on chromosome 9 of the mouse.

Full details of the abovementioned studies are described in the following publications, the disclosure of which are hereby incorporated by reference:
Bement, W. M.; Wirth, J. A.; Mooseker, M. S.: Cloning and mRNA expression of human unconventional myosin-IC: a homologue of amoeboid myosins-1 with a single IQ motif and an SH3 domain. J. Molec. Biol. 243: 356-363, 1994.; and
Hasson, T.; Skowron, J. F.; Gilbert, D. J.; Avraham, K. B.; Perry, W. L.; Bement, W. M.; Anderson, B. L.; Sherr, E. H.; Chen, Z.-Y.; Greene, L. A.; Ward, D. C.; Corey, D. P.; Mooseker.

Further studies establishing the function and utilities of MYO1E are found in John Hopkins OMIM database record ID 601479, and in sited publications numbered 150 and 1615 listed in the bibliography section hereinbelow, which are also hereby incorporated by reference. Rho-associated, Coiled-coil Containing Protein Kinase 2 (ROCK2, Accession XM_{—}038377) is another VGAM35 host target gene. ROCK2 BINDING SITE is HOST TARGET binding site found in the 3′ untranslated region of mRNA encoded by ROCK2, corresponding to a HOST TARGET binding site such as BINDING SITE I, BINDING SITE II or BINDING SITE III. Table 2 illustrates the complementarity of the nucleotide sequences of ROCK2 BINDING SITE, designated SEQ ID:2737, to the nucleotide sequence of VGAM35 RNA, herein designated VGAM RNA, also designated SEQ ID:370.

Another function of VGAM35 is therefore inhibition of Rho-associated, Coiled-coil Containing Protein Kinase 2 (ROCK2, Accession XM_{—}038377), a gene which regulates cytokinesis, smooth muscle contraction, the formation of actin stress fibers and focal adhesions. Accordingly, utilities of VGAM35 include diagnosis, prevention and treatment of diseases and clinical conditions associated with ROCK2. The function of ROCK2 has been established by previous studies. ROCK2 is a serine/threonine kinase that regulates cytokinesis, smooth muscle contraction, the formation of actin stress fibers and focal adhesions, and the activation of the c-fos (OMIM Ref. No. 164810) serum response element. ROCK2, which is an isozyme of ROCK1 (OMIM Ref. No. 601702), is a target for the small GTPase Rho (e.g., 165390). Nakamura et al. (2001) studied the role of Rho in the migration of corneal epithelial cells in rabbit. They detected both ROCK1 (OMIM Ref. No. 601702) and ROCK2 in the corneal epithelium at protein and mRNA levels. They found that exoenzyme C3, a Rho inhibitor, inhibits corneal epithelial migration in a dose-dependent manner and prevents the stimulatory effect of the Rho activator lysophosphatidic acid (LPA). Both cytochalasin B, an inhibitor of actin filament assembly, and ML7, an inhibitor of myosin light chain kinase, also prevent LPA stimulation of epithelial migration. The authors suggested that Rho mediates corneal epithelial migration in response to external stimuli by regulating the organization of the actin cytoskeleton. Rao et al. (2001) investigated the role of Rho kinase in the modulation of aqueous humor outflow facility. The treatment of human trabecular meshwork and canal of Schlemm cells with a Rho kinase-specific inhibitor led to significant but reversible changes in cell shape and decreased actin stress fibers, focal adhesions, and protein phosphotyrosine staining. Based on the Rho kinase inhibitor-induced changes in myosin light chain phosphorylation and actomyosin organization, the authors suggested that cellular relaxation and loss of cell-substratum adhesions in the human trabecular meshwork and canal of Schlemm cells could result in either increased paracellular fluid flow across the canal of Schlemm or altered flow pathway through the juxtacanalicular tissue, thereby lowering resistance to outflow. They suggested Rho kinase as a potential target for the development of drugs to modulate intraocular pressure in glaucoma patients.

Full details of the abovementioned studies are described in the following publications, the disclosure of which are hereby incorporated by reference:
Nakamura, M.; Nagano, T.; Chikama, T.; Nishida, T.: Role of the small GTP-binding protein Rho in epithelial cell migration in the rabbit cornea. Invest. Ophthal. Vis. Sci. 42: 941-947, 2001.; and
Rao, P. V.; Deng, P.-F.; Kumar, J.; Epstein, D. L.: Modulation of aqueous humor outflow facility by the Rho kinase-specific inhibitor Y-27632. Invest. Ophthal. Vis. Sci. 42: 1029-1037.

Further studies establishing the function and utilities of ROCK2 are found in John Hopkins OMIM database record ID 604002, and in sited publications numbered 2136, 2511-251 and 1667 listed in the bibliography section hereinbelow, which are also hereby incorporated by reference. MGC15937 (Accession NM_{—}080661) is another VGAM35 host target gene. MGC15937 BINDING SITE is HOST TARGET binding site found in the 5′ untranslated region of mRNA encoded by MGC15937, corresponding to a HOST TARGET binding site such as BINDING SITE I, BINDING SITE II or BINDING SITE III. Table 2 illustrates the complementarity of the nucleotide sequences of MGC15937 BINDING SITE, designated SEQ ID:2383, to the nucleotide sequence of VGAM35 RNA, herein designated VGAM RNA, also designated SEQ ID:370.

Another function of VGAM35 is therefore inhibition of MGC15937 (Accession NM_{—}080661). Accordingly, utilities of VGAM35 include diagnosis, prevention and treatment of diseases and clinical conditions associated with MGC15937. Serine/threonine Kinase 16 (STK16, Accession XM_{—}050904) is another VGAM35 host target gene. STK16 BINDING SITE is HOST TARGET binding site found in the 5′ untranslated region of mRNA encoded by STK16, corresponding to a HOST TARGET binding site such as BINDING SITE I, BINDING SITE II or BINDING SITE III. Table 2 illustrates the complementarity of the nucleotide sequences of STK16 BINDING SITE, designated SEQ ID:2937, to the nucleotide sequence of VGAM35 RNA, herein designated VGAM RNA, also designated SEQ ID:370.

Another function of VGAM35 is therefore inhibition of Serine/threonine Kinase 16 (STK16, Accession XM_{—}050904). Accordingly, utilities of VGAM35 include diagnosis, prevention and treatment of diseases and clinical conditions associated with STK16. LOC148029 (Accession XM_{—}086014) is another VGAM35 host target gene. LOC148029 BINDING SITE is HOST TARGET binding site found in the 5′ untranslated region of mRNA encoded by LOC148029, corresponding to a HOST TARGET binding site such as BINDING SITE I, BINDING SITE II or BINDING SITE III. Table 2 illustrates the complementarity of the nucleotide sequences of LOC148029 BINDING SITE, designated SEQ ID:3116, to the nucleotide sequence of VGAM35 RNA, herein designated VGAM RNA, also designated SEQ ID:370.

Another function of VGAM35 is therefore inhibition of LOC148029 (Accession XM_{—}086014). Accordingly, utilities of VGAM35 include diagnosis, prevention and treatment of diseases and clinical conditions associated with LOC148029. LOC169436 (Accession XM_{—}095696) is another VGAM35 host target gene. LOC169436 BINDING SITE is HOST TARGET binding site found in the 5′ untranslated region of mRNA encoded by LOC169436, corresponding to a HOST TARGET binding site such as BINDING SITE I, BINDING SITE II or BINDING SITE III. Table 2 illustrates the complementarity of the nucleotide sequences of LOC169436 BINDING SITE, designated SEQ ID:3261, to the nucleotide sequence of VGAM35 RNA, herein designated VGAM RNA, also designated SEQ ID:370.

Another function of VGAM35 is therefore inhibition of LOC169436 (Accession XM_{—}095696). Accordingly, utilities of VGAM35 include diagnosis, prevention and treatment of diseases and clinical conditions associated with LOC169436. LOC256267 (Accession XM_{—}173007) is another VGAM35 host target gene. LOC256267 BINDING SITE is HOST TARGET binding site found in the 3′ untranslated region of mRNA encoded by LOC256267, corresponding to a HOST TARGET binding site such as BINDING SITE I, BINDING SITE II or BINDING SITE III. Table 2 illustrates the complementarity of the nucleotide sequences of LOC256267 BINDING SITE, designated SEQ ID:3713, to the nucleotide sequence of VGAM35 RNA, herein designated VGAM RNA, also designated SEQ ID:370.

Another function of VGAM35 is therefore inhibition of LOC256267 (Accession XM_{—}173007). Accordingly, utilities of VGAM35 include diagnosis, prevention and treatment of diseases and clinical conditions associated with LOC256267. LOC257426 (Accession XM_{—}039451) is another VGAM35 host target gene. LOC257426 BINDING SITE is HOST TARGET binding site found in the 3′ untranslated region of mRNA encoded by LOC257426, corresponding to a HOST TARGET binding site such as BINDING SITE I, BINDING SITE II or BINDING SITE III. Table 2 illustrates the complementarity of the nucleotide sequences of LOC257426 BINDING SITE, designated SEQ ID:2755, to the nucleotide sequence of VGAM35 RNA, herein designated VGAM RNA, also designated SEQ ID:370.

Another function of VGAM35 is therefore inhibition of LOC257426 (Accession XM_{—}039451). Accordingly, utilities of VGAM35 include diagnosis, prevention and treatment of diseases and clinical conditions associated with LOC257426. LOC92466 (Accession XM_{—}045251) is another VGAM35 host target gene. LOC92466 BINDING SITE is HOST TARGET binding site found in the 3′ untranslated region of mRNA encoded by LOC92466, corresponding to a HOST TARGET binding site such as BINDING SITE I, BINDING SITE II or BINDING SITE III. Table 2 illustrates the complementarity of the nucleotide sequences of LOC92466 BINDING SITE, designated SEQ ID:2858, to the nucleotide sequence of VGAM35 RNA, herein designated VGAM RNA, also designated SEQ ID:370.

Another function of VGAM35 is therefore inhibition of LOC92466 (Accession XM_{—}045251). Accordingly, utilities of VGAM35 include diagnosis, prevention and treatment of diseases and clinical conditions associated with LOC92466. FIG. 1 further provides a conceptual description of a novel bioinformatically detected viral gene of the present invention, referred to here as Viral Genomic Address Messenger 36 (VGAM36) viral gene, which modulates expression of respective host target genes thereof, the function and utility of which host target genes is known in the art.

VGAM36 is a novel bioinformatically detected regulatory, non protein coding, viral micro RNA (miRNA) gene. The method by which VGAM36 was detected is described hereinabove with reference to FIGS. 1-8.

VGAM36 gene, herein designated VGAM GENE, is a viral gene contained in the genome of Vaccinia Virus. VGAM36 host target gene, herein designated VGAM HOST TARGET GENE, is a human gene contained in the human genome.

VGAM36 gene encodes a VGAM36 precursor RNA, herein designated VGAM PRECURSOR RNA. Similar to other miRNA genes, and unlike most ordinary genes, VGAM36 precursor RNA does not encode a protein. A nucleotide sequence identical or highly similar to the nucleotide sequence of VGAM36 precursor RNA is designated SEQ ID:22, and is provided hereinbelow with reference to the sequence listing part. Nucleotide sequence SEQ ID:22 is located at position 3549 relative to the genome of Vaccinia Virus.

VGAM36 precursor RNA folds onto itself, forming VGAM36 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM36 folded precursor RNA into VGAM36 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM36 RNA is designated SEQ ID:371, and is provided hereinbelow with reference to the sequence listing part.

VGAM36 host target gene, herein designated VGAM HOST TARGET GENE, encodes a corresponding messenger RNA, VGAM36 host target RNA, herein designated VGAM HOST TARGET RNA. VGAM36 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM36 RNA, herein designated VGAM RNA, binds complementarily to one or more host target binding sites located in untranslated regions of VGAM36 host target RNA, herein designated VGAM HOST TARGET RNA. This complementary binding is due to the fact that the nucleotide sequence of VGAM36 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM36 RNA, herein designated VGAM RNA, may have a different number of host target binding sites in untranslated regions of a VGAM36 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM36 RNA, herein designated VGAM RNA, to host target binding sites on VGAM36 host target RNA, herein designated VGAM HOST TARGET RNA, such as BINDING SITE I, BINDING SITE II and BINDING SITE III, inhibits translation of VGAM36 host target RNA into VGAM36 host target protein, herein designated VGAM HOST TARGET PROTEIN. VGAM host target protein is therefore outlined by a broken line.

It is appreciated that VGAM36 host target gene, herein designated VGAM HOST TARGET GENE, in fact represents a plurality of VGAM36 host target genes. The mRNA of each one of this plurality of VGAM36 host target genes comprises one or more host target binding sites, each having a nucleotide sequence which is at least partly complementary to VGAM36 RNA, herein designated VGAM RNA, and which when bound by VGAM36 RNA causes inhibition of translation of respective one or more VGAM36 host target proteins.

It is further appreciated by one skilled in the art that the mode of translational inhibition illustrated by FIG. 1 with specific reference to translational inhibition exerted by VGAM36 gene, herein designated VGAM GENE, on one or more VGAM36 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM36 is inhibition of expression of host target genes, as part of a novel viral mechanism of attacking a host. Accordingly, utilities of VGAM36 include diagnosis, prevention and treatment of viral infection by Vaccinia Virus. Specific functions, and accordingly utilities, of VGAM36 correlate with, and may be deduced from, the identity of the host target genes which VGAM36 binds and inhibits, and the function of these host target genes, as elaborated hereinbelow.

Nucleotide sequences of the VGAM36 precursor RNA, herein designated VGAM PRECURSOR RNA, and of the ‘diced’ VGAM36 RNA, herein designated VGAM RNA, and a schematic representation of the secondary folding of VGAM36 folded precursor RNA, herein designated VGAM FOLDED PRECURSOR RNA, of VGAM36 are further described hereinbelow with reference to Table 1.

Nucleotide sequences of host target binding sites, such as BINDING SITE-I, BINDING SITE-II and BINDING SITE-III of FIG. 1, found on VGAM36 host target RNA, and schematic representation of the complementarity of each of these host target binding sites to VGAM36 RNA, herein designated VGAM RNA, are described hereinbelow with reference to Table 2.

As mentioned hereinabove with reference to FIG. 1, a function of VGAM36 gene, herein designated VGAM is inhibition of expression of VGAM36 target genes. It is appreciated that specific functions, and accordingly utilities, of VGAM36 correlate with, and may be deduced from, the identity of the target genes which VGAM36 binds and inhibits, and the function of these target genes, as elaborated hereinbelow.

Membrane Metallo-endopeptidase (neutral endopeptidase, enkephalinase, CALLA, CD10) (MME, Accession NM_{—}000902) is a VGAM36 host target gene. MME BINDING SITE1 through MME BINDING SITE4 are HOST TARGET binding sites found in untranslated regions of mRNA encoded by MME, corresponding to HOST TARGET binding sites such as BINDING SITE I, BINDING SITE II or BINDING SITE III. Table 2 illustrates the complementarity of the nucleotide sequences of MME BINDING SITE1 through MME BINDING SITE4, designated SEQ ID:785, SEQ ID:1377, SEQ ID:1378 and SEQ ID:1379 respectively, to the nucleotide sequence of VGAM36 RNA, herein designated VGAM RNA, also designated SEQ ID:371.

A function of VGAM36 is therefore inhibition of Membrane Metallo-endopeptidase (neutral endopeptidase, enkephalinase, CALLA, CD10) (MME, Accession NM_{—}000902), a gene which is thermolysin-like specificity. Accordingly, utilities of VGAM36 include diagnosis, prevention and treatment of diseases and clinical conditions associated with MME. The function of MME has been established by previous studies. Common acute lymphocytic leukemia antigen is an important cell surface marker in the diagnosis of human acute lymphocytic leukemia (ALL). It is present on leukemic cells of pre-B phenotype, which represent 85% of cases of ALL. CALLA is not restricted to leukemic cells, however, and is found on a variety of normal tissues. CALLA is a glycoprotein that is particularly abundant in kidney, where it is present on the brush border of proximal tubules and on glomerular epithelium. Letarte et al. (1988) cloned a cDNA coding for CALLA and showed that the amino acid sequence deduced from the cDNA sequence is identical to that of human membrane-associated neutral endopeptidase (NEP; EC 3.4.24.11), also known as enkephalinase. NEP cleaves peptides at the amino side of hydrophobic residues and inactivates several peptide hormones including glucagon, enkephalins, substance P, neurotensin, oxytocin, and bradykinin. By cDNA transfection analysis, Shipp et al. (1989) confirmed that CALLA is a functional neutral endopeptidase of the type that has previously been called enkephalinase. Barker et al. (1989) demonstrated that the CALLA gene, which encodes a 100-kD type II transmembrane glycoprotein, exists in a single copy of greater than 45 kb which is not rearranged in malignancies expressing cell surface CALLA. D'Adamio et al. (1989) demonstrated that the CALLA gene spans more than 80 kb and is composed of 24 exons. Animal model experiments lend further support to the function of MME. Amyloid-beta peptide (OMIM Ref. No. 104760), the pathogenic agent of Alzheimer disease (OMIM Ref. No. 104300), is a physiologic metabolite in the brain. Iwata et al. (2001) examined the role of neprilysin, a candidate amyloid-beta degrading peptidase, in the metabolism using neprilysin gene-disrupted mice. Neprilysin deficiency resulted in defects both in the degradation of exogenously administered amyloid-beta and in the metabolic suppression of the endogenous amyloid-beta levels in a gene dose-dependent manner. The regional levels of amyloid-beta in the neprilysin-deficient mouse brain were in the distinct order of hippocampus, cortex, thalamus/striatum, and cerebellum, where hippocampus has the highest level and cerebellum the lowest, correlating with the vulnerability to amyloid-beta deposition in brains of humans with Alzheimer disease. Iwata et al. (2001) concluded that even partial downregulation of neprilysin activity, which could be caused by aging, can contribute to Alzheimer disease by promoting amyloid-beta accumulation.

It is appreciated that the abovementioned animal model for MME is acknowledged by those skilled in the art as a scientifically valid animal model, as can be further appreciated from the publications sited hereinbelow.

Full details of the abovementioned studies are described in the following publications, the disclosure of which are hereby incorporated by reference:
Iwata, N.; Tsubuki, S.; Takaki, Y.; Shirotani, K.; Lu, B.; Gerard, N. P.; Gerard, C.; Hama, E.; Lee, H.-J.; Saido, T. C.: Metabolic regulation of brain A-beta by neprilysin. Science 292: 1550-1552, 2001.; and
Letarte, M.; Vera, S.; Tran, R.; Addis, J. B. L.; Onizuka, R. J.; Quackenbush, E. J.; Jongeneel, C. V.; McInnes, R. R.: Common acute lymphocytic leukemia antigen is identical to neutr.

Further studies establishing the function and utilities of MME are found in John Hopkins OMIM database record ID 120520, and in sited publications numbered 2865-2867, 91 and 2868-2870 listed in the bibliography section hereinbelow, which are also hereby incorporated by reference. FLJ11275 (Accession NM_{—}018376) is another VGAM36 host target gene. FLJ11275 BINDING SITE is HOST TARGET binding site found in the 3′ untranslated region of mRNA encoded by FLJ11275, corresponding to a HOST TARGET binding site such as BINDING SITE I, BINDING SITE II or BINDING SITE III. Table 2 illustrates the complementarity of the nucleotide sequences of FLJ11275 BINDING SITE, designated SEQ ID:1821, to the nucleotide sequence of VGAM36 RNA, herein designated VGAM RNA, also designated SEQ ID:371.

Another function of VGAM36 is therefore inhibition of FLJ11275 (Accession NM_{—}018376). Accordingly, utilities of VGAM36 include diagnosis, prevention and treatment of diseases and clinical conditions associated with FLJ11275. Golgi Phosphoprotein 3 (coat-protein) (GOLPH3, Accession NM_{—}022130) is another VGAM36 host target gene. GOLPH3 BINDING SITE is HOST TARGET binding site found in the 3′ untranslated region of mRNA encoded by GOLPH3, corresponding to a HOST TARGET binding site such as BINDING SITE I, BINDING SITE II or BINDING SITE III. Table 2 illustrates the complementarity of the nucleotide sequences of GOLPH3 BINDING SITE, designated SEQ ID:1981, to the nucleotide sequence of VGAM36 RNA, herein designated VGAM RNA, also designated SEQ ID:371.

Another function of VGAM36 is therefore inhibition of Golgi Phosphoprotein 3 (coat-protein) (GOLPH3, Accession NM_{—}022130). Accordingly, utilities of VGAM36 include diagnosis, prevention and treatment of diseases and clinical conditions associated with GOLPH3. KIAA0563 (Accession NM_{—}014834) is another VGAM36 host target gene. KIAA0563 BINDING SITE is HOST TARGET binding site found in the 5′ untranslated region of mRNA encoded by KIAA0563, corresponding to a HOST TARGET binding site such as BINDING SITE I, BINDING SITE II or BINDING SITE III. Table 2 illustrates the complementarity of the nucleotide sequences of KIAA0563 BINDING SITE, designated SEQ ID:1565, to the nucleotide sequence of VGAM36 RNA, herein designated VGAM RNA, also designated SEQ ID:371.

Another function of VGAM36 is therefore inhibition of KIAA0563 (Accession NM_{—}014834). Accordingly, utilities of VGAM36 include diagnosis, prevention and treatment of diseases and clinical conditions associated with KIAA0563. TAF2 RNA Polymerase II, TATA Box Binding Protein (TBP)-associated Factor, 150 kDa (TAF2, Accession NM_{—}003184) is another VGAM36 host target gene. TAF2 BINDING SITE is HOST TARGET binding site found in the 3′ untranslated region of mRNA encoded by TAF2, corresponding to a HOST TARGET binding site such as BINDING SITE I, BINDING SITE II or BINDING SITE III. Table 2 illustrates the complementarity of the nucleotide sequences of TAF2 BINDING SITE, designated SEQ ID:997, to the nucleotide sequence of VGAM36 RNA, herein designated VGAM RNA, also designated SEQ ID:371.

Another function of VGAM36 is therefore inhibition of TAF2 RNA Polymerase II, TATA Box Binding Protein (TBP)-associated Factor, 150 kDa (TAF2, Accession NM_{—}003184). Accordingly, utilities of VGAM36 include diagnosis, prevention and treatment of diseases and clinical conditions associated with TAF2. LOC147071 (Accession XM_{—}054031) is another VGAM36 host target gene. LOC147071 BINDING SITE is HOST TARGET binding site found in the 5′ untranslated region of mRNA encoded by LOC147071, corresponding to a HOST TARGET binding site such as BINDING SITE I, BINDING SITE II or BINDING SITE III. Table 2 illustrates the complementarity of the nucleotide sequences of LOC147071 BINDING SITE, designated SEQ ID:2965, to the nucleotide sequence of VGAM36 RNA, herein designated VGAM RNA, also designated SEQ ID:371.

Another function of VGAM36 is therefore inhibition of LOC147071 (Accession XM_{—}054031). Accordingly, utilities of VGAM36 include diagnosis, prevention and treatment of diseases and clinical conditions associated with LOC147071. LOC147639 (Accession XM_{—}085822) is another VGAM36 host target gene. LOC147639 BINDING SITE is HOST TARGET binding site found in the 5′ untranslated region of mRNA encoded by LOC147639, corresponding to a HOST TARGET binding site such as BINDING SITE I, BINDING SITE II or BINDING SITE III. Table 2 illustrates the complementarity of the nucleotide sequences of LOC147639 BINDING SITE, designated SEQ ID:3110, to the nucleotide sequence of VGAM36 RNA, herein designated VGAM RNA, also designated SEQ ID:371.

Another function of VGAM36 is therefore inhibition of LOC147639 (Accession XM_{—}085822). Accordingly, utilities of VGAM36 include diagnosis, prevention and treatment of diseases and clinical conditions associated with LOC147639. LOC201173 (Accession XM_{—}113312) is another VGAM36 host target gene. LOC201173 BINDING SITE is HOST TARGET binding site found in the 5′ untranslated region of mRNA encoded by LOC201173, corresponding to a HOST TARGET binding site such as BINDING SITE I, BINDING SITE II or BINDING SITE III. Table 2 illustrates the complementarity of the nucleotide sequences of LOC201173 BINDING SITE, designated SEQ ID:3405, to the nucleotide sequence of VGAM36 RNA, herein designated VGAM RNA, also designated SEQ ID:371.

Another function of VGAM36 is therefore inhibition of LOC201173 (Accession XM_{—}113312). Accordingly, utilities of VGAM36 include diagnosis, prevention and treatment of diseases and clinical conditions associated with LOC201173. LOC201220 (Accession XM_{—}113321) is another VGAM36 host target gene. LOC201220 BINDING SITE is HOST TARGET binding site found in the 5′ untranslated region of mRNA encoded by LOC201220, corresponding to a HOST TARGET binding site such as BINDING SITE I, BINDING SITE II or BINDING SITE III. Table 2 illustrates the complementarity of the nucleotide sequences of LOC201220 BINDING SITE, designated SEQ ID:3406, to the nucleotide sequence of VGAM36 RNA, herein designated VGAM RNA, also designated SEQ ID:371.

Another function of VGAM36 is therefore inhibition of LOC201220 (Accession XM_{—}113321). Accordingly, utilities of VGAM36 include diagnosis, prevention and treatment of diseases and clinical conditions associated with LOC201220. FIG. 1 further provides a conceptual description of a novel bioinformatically detected viral gene of the present invention, referred to here as Viral Genomic Address Messenger 37 (VGAM37) viral gene, which modulates expression of respective host target genes thereof, the function and utility of which host target genes is known in the art.

VGAM37 is a novel bioinformatically detected regulatory, non protein coding, viral micro RNA (miRNA) gene. The method by which VGAM37 was detected is described hereinabove with reference to FIGS. 1-8.

VGAM37 gene, herein designated VGAM GENE, is a viral gene contained in the genome of Vaccinia Virus. VGAM37 host target gene, herein designated VGAM HOST TARGET GENE, is a human gene contained in the human genome.

VGAM37 gene encodes a VGAM37 precursor RNA, herein designated VGAM PRECURSOR RNA. Similar to other miRNA genes, and unlike most ordinary genes, VGAM37 precursor RNA does not encode a protein. A nucleotide sequence identical or highly similar to the nucleotide sequence of VGAM37 precursor RNA is designated SEQ ID:23, and is provided hereinbelow with reference to the sequence listing part. Nucleotide sequence SEQ ID:23 is located at position 3549 relative to the genome of Vaccinia Virus.

VGAM37 precursor RNA folds onto itself, forming VGAM37 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM37 folded precursor RNA into VGAM37 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM37 RNA is designated SEQ ID:372, and is provided hereinbelow with reference to the sequence listing part.

VGAM37 host target gene, herein designated VGAM HOST TARGET GENE, encodes a corresponding messenger RNA, VGAM37 host target RNA, herein designated VGAM HOST TARGET RNA. VGAM37 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM37 RNA, herein designated VGAM RNA, binds complementarily to one or more host target binding sites located in untranslated regions of VGAM37 host target RNA, herein designated VGAM HOST TARGET RNA. This complementary binding is due to the fact that the nucleotide sequence of VGAM37 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM37 RNA, herein designated VGAM RNA, may have a different number of host target binding sites in untranslated regions of a VGAM37 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM37 RNA, herein designated VGAM RNA, to host target binding sites on VGAM37 host target RNA, herein designated VGAM HOST TARGET RNA, such as BINDING SITE I, BINDING SITE II and BINDING SITE III, inhibits translation of VGAM37 host target RNA into VGAM37 host target protein, herein designated VGAM HOST TARGET PROTEIN. VGAM host target protein is therefore outlined by a broken line.

It is appreciated that VGAM37 host target gene, herein designated VGAM HOST TARGET GENE, in fact represents a plurality of VGAM37 host target genes. The mRNA of each one of this plurality of VGAM37 host target genes comprises one or more host target binding sites, each having a nucleotide sequence which is at least partly complementary to VGAM37 RNA, herein designated VGAM RNA, and which when bound by VGAM37 RNA causes inhibition of translation of respective one or more VGAM37 host target proteins.

It is further appreciated by one skilled in the art that the mode of translational inhibition illustrated by FIG. 1 with specific reference to translational inhibition exerted by VGAM37 gene, herein designated VGAM GENE, on one or more VGAM37 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM37 is inhibition of expression of host target genes, as part of a novel viral mechanism of attacking a host. Accordingly, utilities of VGAM37 include diagnosis, prevention and treatment of viral infection by Vaccinia Virus. Specific functions, and accordingly utilities, of VGAM37 correlate with, and may be deduced from, the identity of the host target genes which VGAM37 binds and inhibits, and the function of these host target genes, as elaborated hereinbelow.

Nucleotide sequences of the VGAM37 precursor RNA, herein designated VGAM PRECURSOR RNA, and of the ‘diced’ VGAM37 RNA, herein designated VGAM RNA, and a schematic representation of the secondary folding of VGAM37 folded precursor RNA, herein designated VGAM FOLDED PRECURSOR RNA, of VGAM37 are further described hereinbelow with reference to Table 1.

Nucleotide sequences of host target binding sites, such as BINDING SITE-I, BINDING SITE-II and BINDING SITE-III of FIG. 1, found on VGAM37 host target RNA, and schematic representation of the complementarity of each of these host target binding sites to VGAM37 RNA, herein designated VGAM RNA, are described hereinbelow with reference to Table 2.

As mentioned hereinabove with reference to FIG. 1, a function of VGAM37 gene, herein designated VGAM is inhibition of expression of VGAM37 target genes. It is appreciated that specific functions, and accordingly utilities, of VGAM37 correlate with, and may be deduced from, the identity of the target genes which VGAM37 binds and inhibits, and the function of these target genes, as elaborated hereinbelow.

Membrane Metallo-endopeptidase (neutral endopeptidase, enkephalinase, CALLA, CD10) (MME, Accession NM_{—}000902) is a VGAM37 host target gene. MME BINDING SITE1 through MME BINDING SITE4 are HOST TARGET binding sites found in untranslated regions of mRNA encoded by MME, corresponding to HOST TARGET binding sites such as BINDING SITE I, BINDING SITE II or BINDING SITE III. Table 2 illustrates the complementarity of the nucleotide sequences of MME BINDING SITE1 through MME BINDING SITE4, designated SEQ ID:785, SEQ ID:1377, SEQ ID:1378 and SEQ ID:1379 respectively, to the nucleotide sequence of VGAM37 RNA, herein designated VGAM RNA, also designated SEQ ID:372.

A function of VGAM37 is therefore inhibition of Membrane Metallo-endopeptidase (neutral endopeptidase, enkephalinase, CALLA, CD10) (MME, Accession NM_{—}000902), a gene which is thermolysin-like specificity. Accordingly, utilities of VGAM37 include diagnosis, prevention and treatment of diseases and clinical conditions associated with MME. The function of MME and its association with various diseases and clinical conditions, has been established by previous studies, as described hereinabove with reference to VGAM36. FLJ11275 (Accession NM_{—}018376) is another VGAM37 host target gene. FLJ11275 BINDING SITE is HOST TARGET binding site found in the 3′ untranslated region of mRNA encoded by FLJ11275, corresponding to a HOST TARGET binding site such as BINDING SITE I, BINDING SITE II or BINDING SITE III. Table 2 illustrates the complementarity of the nucleotide sequences of FLJ11275 BINDING SITE, designated SEQ ID:1821, to the nucleotide sequence of VGAM37 RNA, herein designated VGAM RNA, also designated SEQ ID:372.

Another function of VGAM37 is therefore inhibition of FLJ11275 (Accession NM_{—}018376). Accordingly, utilities of VGAM37 include diagnosis, prevention and treatment of diseases and clinical conditions associated with FLJ11275. Golgi Phosphoprotein 3 (coat-protein) (GOLPH3, Accession NM_{—}022130) is another VGAM37 host target gene. GOLPH3 BINDING SITE is HOST TARGET binding site found in the 3′ untranslated region of mRNA encoded by GOLPH3, corresponding to a HOST TARGET binding site such as BINDING SITE I, BINDING SITE II or BINDING SITE III. Table 2 illustrates the complementarity of the nucleotide sequences of GOLPH3 BINDING SITE, designated SEQ ID:1981, to the nucleotide sequence of VGAM37 RNA, herein designated VGAM RNA, also designated SEQ ID:372.

Another function of VGAM37 is therefore inhibition of Golgi Phosphoprotein 3 (coat-protein) (GOLPH3, Accession NM_{—}022130). Accordingly, utilities of VGAM37 include diagnosis, prevention and treatment of diseases and clinical conditions associated with GOLPH3. KIAA0563 (Accession NM_{—}014834) is another VGAM37 host target gene. KIAA0563 BINDING SITE is HOST TARGET binding site found in the 5′ untranslated region of mRNA encoded by KIAA0563, corresponding to a HOST TARGET binding site such as BINDING SITE I, BINDING SITE II or BINDING SITE III. Table 2 illustrates the complementarity of the nucleotide sequences of KIAA0563 BINDING SITE, designated SEQ ID:1565, to the nucleotide sequence of VGAM37 RNA, herein designated VGAM RNA, also designated SEQ ID:372.

Another function of VGAM37 is therefore inhibition of KIAA0563 (Accession NM_{—}014834). Accordingly, utilities of VGAM37 include diagnosis, prevention and treatment of diseases and clinical conditions associated with KIAA0563. TAF2 RNA Polymerase II, TATA Box Binding Protein (TBP)-associated Factor, 150 kDa (TAF2, Accession NM_{—}003184) is another VGAM37 host target gene. TAF2 BINDING SITE is HOST TARGET binding site found in the 3′ untranslated region of mRNA encoded by TAF2, corresponding to a HOST TARGET binding site such as BINDING SITE I, BINDING SITE II or BINDING SITE III. Table 2 illustrates the complementarity of the nucleotide sequences of TAF2 BINDING SITE, designated SEQ ID:997, to the nucleotide sequence of VGAM37 RNA, herein designated VGAM RNA, also designated SEQ ID:372.

Another function of VGAM37 is therefore inhibition of TAF2 RNA Polymerase II, TATA Box Binding Protein (TBP)-associated Factor, 150 kDa (TAF2, Accession NM_{—}003184). Accordingly, utilities of VGAM37 include diagnosis, prevention and treatment of diseases and clinical conditions associated with TAF2. LOC147071 (Accession XM_{—}054031) is another VGAM37 host target gene. LOC147071 BINDING SITE is HOST TARGET binding site found in the 5′ untranslated region of mRNA encoded by LOC147071, corresponding to a HOST TARGET binding site such as BINDING SITE I, BINDING SITE II or BINDING SITE III. Table 2 illustrates the complementarity of the nucleotide sequences of LOC147071 BINDING SITE, designated SEQ ID:2965, to the nucleotide sequence of VGAM37 RNA, herein designated VGAM RNA, also designated SEQ ID:372.

Another function of VGAM37 is therefore inhibition of LOC147071 (Accession XM_{—}054031). Accordingly, utilities of VGAM37 include diagnosis, prevention and treatment of diseases and clinical conditions associated with LOC147071. LOC147639 (Accession XM_{—}085822) is another VGAM37 host target gene. LOC147639 BINDING SITE is HOST TARGET binding site found in the 5′ untranslated region of mRNA encoded by LOC147639, corresponding to a HOST TARGET binding site such as BINDING SITE I, BINDING SITE II or BINDING SITE III. Table 2 illustrates the complementarity of the nucleotide sequences of LOC147639 BINDING SITE, designated SEQ ID:3110, to the nucleotide sequence of VGAM37 RNA, herein designated VGAM RNA, also designated SEQ ID:372.

Another function of VGAM37 is therefore inhibition of LOC147639 (Accession XM_{—}085822). Accordingly, utilities of VGAM37 include diagnosis, prevention and treatment of diseases and clinical conditions associated with LOC147639. LOC201173 (Accession XM_{—}113312) is another VGAM37 host target gene. LOC201173 BINDING SITE is HOST TARGET binding site found in the 5′ untranslated region of mRNA encoded by LOC201173, corresponding to a HOST TARGET binding site such as BINDING SITE I, BINDING SITE II or BINDING SITE III. Table 2 illustrates the complementarity of the nucleotide sequences of LOC201173 BINDING SITE, designated SEQ ID:3405, to the nucleotide sequence of VGAM37 RNA, herein designated VGAM RNA, also designated SEQ ID:372.

Another function of VGAM37 is therefore inhibition of LOC201173 (Accession XM_{—}113312). Accordingly, utilities of VGAM37 include diagnosis, prevention and treatment of diseases and clinical conditions associated with LOC201173. LOC201220 (Accession XM_{—}113321) is another VGAM37 host target gene. LOC201220 BINDING SITE is HOST TARGET binding site found in the 5′ untranslated region of mRNA encoded by LOC201220, corresponding to a HOST TARGET binding site such as BINDING SITE I, BINDING SITE II or BINDING SITE III. Table 2 illustrates the complementarity of the nucleotide sequences of LOC201220 BINDING SITE, designated SEQ ID:3406, to the nucleotide sequence of VGAM37 RNA, herein designated VGAM RNA, also designated SEQ ID:372.

Another function of VGAM37 is therefore inhibition of LOC201220 (Accession XM_{—}113321). Accordingly, utilities of VGAM37 include diagnosis, prevention and treatment of diseases and clinical conditions associated with LOC201220. FIG. 1 further provides a conceptual description of a novel bioinformatically detected viral gene of the present invention, referred to here as Viral Genomic Address Messenger 38 (VGAM38) viral gene, which modulates expression of respective host target genes thereof, the function and utility of which host target genes is known in the art.

VGAM38 is a novel bioinformatically detected regulatory, non protein coding, viral micro RNA (miRNA) gene. The method by which VGAM38 was detected is described hereinabove with reference to FIGS. 1-8.

VGAM38 gene, herein designated VGAM GENE, is a viral gene contained in the genome of Vaccinia Virus. VGAM38 host target gene, herein designated VGAM HOST TARGET GENE, is a human gene contained in the human genome.

VGAM38 gene encodes a VGAM38 precursor RNA, herein designated VGAM PRECURSOR RNA. Similar to other miRNA genes, and unlike most ordinary genes, VGAM38 precursor RNA does not encode a protein. A nucleotide sequence identical or highly similar to the nucleotide sequence of VGAM38 precursor RNA is designated SEQ ID:24, and is provided hereinbelow with reference to the sequence listing part. Nucleotide sequence SEQ ID:24 is located at position 2981 relative to the genome of Vaccinia Virus.

VGAM38 precursor RNA folds onto itself, forming VGAM38 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM38 folded precursor RNA into VGAM38 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM38 RNA is designated SEQ ID:373, and is provided hereinbelow with reference to the sequence listing part.

VGAM38 host target gene, herein designated VGAM HOST TARGET GENE, encodes a corresponding messenger RNA, VGAM38 host target RNA, herein designated VGAM HOST TARGET RNA. VGAM38 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM38 RNA, herein designated VGAM RNA, binds complementarily to one or more host target binding sites located in untranslated regions of VGAM38 host target RNA, herein designated VGAM HOST TARGET RNA. This complementary binding is due to the fact that the nucleotide sequence of VGAM38 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM38 RNA, herein designated VGAM RNA, may have a different number of host target binding sites in untranslated regions of a VGAM38 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM38 RNA, herein designated VGAM RNA, to host target binding sites on VGAM38 host target RNA, herein designated VGAM HOST TARGET RNA, such as BINDING SITE I, BINDING SITE II and BINDING SITE III, inhibits translation of VGAM38 host target RNA into VGAM38 host target protein, herein designated VGAM HOST TARGET PROTEIN. VGAM host target protein is therefore outlined by a broken line.

It is appreciated that VGAM38 host target gene, herein designated VGAM HOST TARGET GENE, in fact represents a plurality of VGAM38 host target genes. The mRNA of each one of this plurality of VGAM38 host target genes comprises one or more host target binding sites, each having a nucleotide sequence which is at least partly complementary to VGAM38 RNA, herein designated VGAM RNA, and which when bound by VGAM38 RNA causes inhibition of translation of respective one or more VGAM38 host target proteins.

It is further appreciated by one skilled in the art that the mode of translational inhibition illustrated by FIG. 1 with specific reference to translational inhibition exerted by VGAM38 gene, herein designated VGAM GENE, on one or more VGAM38 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM38 is inhibition of expression of host target genes, as part of a novel viral mechanism of attacking a host. Accordingly, utilities of VGAM38 include diagnosis, prevention and treatment of viral infection by Vaccinia Virus. Specific functions, and accordingly utilities, of VGAM38 correlate with, and may be deduced from, the identity of the host target genes which VGAM38 binds and inhibits, and the function of these host target genes, as elaborated hereinbelow.

Nucleotide sequences of the VGAM38 precursor RNA, herein designated VGAM PRECURSOR RNA, and of the ‘diced’ VGAM38 RNA, herein designated VGAM RNA, and a schematic representation of the secondary folding of VGAM38 folded precursor RNA, herein designated VGAM FOLDED PRECURSOR RNA, of VGAM38 are further described hereinbelow with reference to Table 1.

Nucleotide sequences of host target binding sites, such as BINDING SITE-I, BINDING SITE-II and BINDING SITE-III of FIG. 1, found on VGAM38 host target RNA, and schematic representation of the complementarity of each of these host target binding sites to VGAM38 RNA, herein designated VGAM RNA, are described hereinbelow with reference to Table 2.

As mentioned hereinabove with reference to FIG. 1, a function of VGAM38 gene, herein designated VGAM is inhibition of expression of VGAM38 target genes. It is appreciated that specific functions, and accordingly utilities, of VGAM38 correlate with, and may be deduced from, the identity of the target genes which VGAM38 binds and inhibits, and the function of these target genes, as elaborated hereinbelow.

MAP/microtubule Affinity-regulating Kinase 1 (MARK1, Accession NM_{—}018650) is a VGAM38 host target gene. MARK1 BINDING SITE is HOST TARGET binding site found in the 3′ untranslated region of mRNA encoded by MARK1, corresponding to a HOST TARGET binding site such as BINDING SITE I, BINDING SITE II or BINDING SITE III. Table 2 illustrates the complementarity of the nucleotide sequences of MARK1 BINDING SITE, designated SEQ ID:1857, to the nucleotide sequence of VGAM38 RNA, herein designated VGAM RNA, also designated SEQ ID:373.

A function of VGAM38 is therefore inhibition of MAP/microtubule Affinity-regulating Kinase 1 (MARK1, Accession NM_{—}018650), a gene which phosphorylate microtubule-associated proteins and trigger microtubule disruption. Accordingly, utilities of VGAM38 include diagnosis, prevention and treatment of diseases and clinical conditions associated with MARK1. The function of MARK1 has been established by previous studies. The microtubule array has a central role in the regulation of cell shape and polarity during differentiation, chromosome partitioning at mitosis, and intracellular transport. Microtubules undergo rearrangements involving rapid transitions between stable and dynamic states during these processes. By RT-PCR of rat and human brain mRNA, followed by PCR with primers based on the rat sequences, Drewes et al. (1997) cloned human genes for MARK1 and MARK2 (see OMIM Ref. No. EMK1, 600526). The rat protein has 793 amino acids. Northern blot analysis with rat probes revealed expression of a 4.1-kb transcript with highest levels in heart and muscle. By searching for sequences encoding large proteins expressed in brain, Nagase et al. (2000) identified a partial cDNA encoding MARK1, which they termed KIAA1477. The 870-amino acid protein is 90% identical to the rat serine/threonine kinase Mark1. RT-PCR analysis detected ubiquitous expression at highest levels in testis and brain. Within brain, highest levels were detected in hippocampus. By radiation hybrid analysis, Nagase et al. (2000) mapped the MARK1 gene to chromosome 1.

Full details of the abovementioned studies are described in the following publications, the disclosure of which are hereby incorporated by reference:
Drewes, G.; Ebneth, A.; Preuss, U.; Mandelkow, E. M.; Mandelkow, E.: MARK, a novel family of protein kinases that phosphorylate microtubule-associated proteins and trigger microtubule disruption. Cell 89: 297-308, 1997.; and
Nagase, T.; Kikuno, R.; Ishikawa, K.; Hirosawa, M.; Ohara, O.: Prediction of the coding sequences of unidentified human genes. XVII. The complete sequences of 100 new cDNA clones from.

Further studies establishing the function and utilities of MARK1 are found in John Hopkins OMIM database record ID 606511, and in sited publications numbered 1405-1406 listed in the bibliography section hereinbelow, which are also hereby incorporated by reference. FIG. 1 further provides a conceptual description of a novel bioinformatically detected viral gene of the present invention, referred to here as Viral Genomic Address Messenger 39 (VGAM39) viral gene, which modulates expression of respective host target genes thereof, the function and utility of which host target genes is known in the art.

VGAM39 is a novel bioinformatically detected regulatory, non protein coding, viral micro RNA (miRNA) gene. The method by which VGAM39 was detected is described hereinabove with reference to FIGS. 1-8.

VGAM39 gene, herein designated VGAM GENE, is a viral gene contained in the genome of Vaccinia Virus. VGAM39 host target gene, herein designated VGAM HOST TARGET GENE, is a human gene contained in the human genome.

VGAM39 gene encodes a VGAM39 precursor RNA, herein designated VGAM PRECURSOR RNA. Similar to other miRNA genes, and unlike most ordinary genes, VGAM39 precursor RNA does not encode a protein. A nucleotide sequence identical or highly similar to the nucleotide sequence of VGAM39 precursor RNA is designated SEQ ID:25, and is provided hereinbelow with reference to the sequence listing part. Nucleotide sequence SEQ ID:25 is located at position 963 relative to the genome of Vaccinia Virus.

VGAM39 precursor RNA folds onto itself, forming VGAM39 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM39 folded precursor RNA into VGAM39 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM39 RNA is designated SEQ ID:374, and is provided hereinbelow with reference to the sequence listing part.

VGAM39 host target gene, herein designated VGAM HOST TARGET GENE, encodes a corresponding messenger RNA, VGAM39 host target RNA, herein designated VGAM HOST TARGET RNA. VGAM39 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM39 RNA, herein designated VGAM RNA, binds complementarily to one or more host target binding sites located in untranslated regions of VGAM39 host target RNA, herein designated VGAM HOST TARGET RNA. This complementary binding is due to the fact that the nucleotide sequence of VGAM39 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM39 RNA, herein designated VGAM RNA, may have a different number of host target binding sites in untranslated regions of a VGAM39 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM39 RNA, herein designated VGAM RNA, to host target binding sites on VGAM39 host target RNA, herein designated VGAM HOST TARGET RNA, such as BINDING SITE I, BINDING SITE II and BINDING SITE III, inhibits translation of VGAM39 host target RNA into VGAM39 host target protein, herein designated VGAM HOST TARGET PROTEIN. VGAM host target protein is therefore outlined by a broken line.

It is appreciated that VGAM39 host target gene, herein designated VGAM HOST TARGET GENE, in fact represents a plurality of VGAM39 host target genes. The mRNA of each one of this plurality of VGAM39 host target genes comprises one or more host target binding sites, each having a nucleotide sequence which is at least partly complementary to VGAM39 RNA, herein designated VGAM RNA, and which when bound by VGAM39 RNA causes inhibition of translation of respective one or more VGAM39 host target proteins.

It is further appreciated by one skilled in the art that the mode of translational inhibition illustrated by FIG. 1 with specific reference to translational inhibition exerted by VGAM39 gene, herein designated VGAM GENE, on one or more VGAM39 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM39 is inhibition of expression of host target genes, as part of a novel viral mechanism of attacking a host. Accordingly, utilities of VGAM39 include diagnosis, prevention and treatment of viral infection by Vaccinia Virus. Specific functions, and accordingly utilities, of VGAM39 correlate with, and may be deduced from, the identity of the host target genes which VGAM39 binds and inhibits, and the function of these host target genes, as elaborated hereinbelow.

Nucleotide sequences of the VGAM39 precursor RNA, herein designated VGAM PRECURSOR RNA, and of the ‘diced’ VGAM39 RNA, herein designated VGAM RNA, and a schematic representation of the secondary folding of VGAM39 folded precursor RNA, herein designated VGAM FOLDED PRECURSOR RNA, of VGAM39 are further described hereinbelow with reference to Table 1.

Nucleotide sequences of host target binding sites, such as BINDING SITE-I, BINDING SITE-II and BINDING SITE-III of FIG. 1, found on VGAM39 host target RNA, and schematic representation of the complementarity of each of these host target binding sites to VGAM39 RNA, herein designated VGAM RNA, are described hereinbelow with reference to Table 2.

As mentioned hereinabove with reference to FIG. 1, a function of VGAM39 gene, herein designated VGAM is inhibition of expression of VGAM39 target genes. It is appreciated that specific functions, and accordingly utilities, of VGAM39 correlate with, and may be deduced from, the identity of the target genes which VGAM39 binds and inhibits, and the function of these target genes, as elaborated hereinbelow.

Dickkopf Homolog 1 (Xenopus laevis) (DKK1, Accession NM_{—}012242) is a VGAM39 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM39 RNA, herein designated VGAM RNA, also designated SEQ ID:374.

A function of VGAM39 is therefore inhibition of Dickkopf Homolog 1 (Xenopus laevis) (DKK1, Accession NM_{—}012242), a gene which is a growth factor and inhibits Wnt signaling. Accordingly, utilities of VGAM39 include diagnosis, prevention and treatment of diseases and clinical conditions associated with DKK1. The function of DKK1 has been established by previous studies. Roessler et al. (2000) identified 4 heterozygous sequence alterations in the DKK1 gene in patients with holoprosencephaly (HPE; OMIM Ref. No. 236100). Functional analysis of the alterations, however, revealed preserved activity in frog ectopic head assays, suggesting a limited role for DKK1 in HPE. Lrp6 (OMIM Ref. No. 603507) is required during Wnt/beta-catenin signaling in Drosophila, Xenopus, and mouse, possibly acting as a coreceptor for Wnt. Mao et al. (2001) showed that LRP6 is a specific, high affinity receptor for DKK1 and DKK2. DKK1 blocks Lrp6-mediated Wnt/beta-catenin signaling by interacting with domains that are distinct from those required for Wnt/frizzled (OMIM Ref. No. 600667) interaction. DKK1 and Lrp6 interact antagonistically during embryogenic head induction in Xenopus where Lrp6 promotes the posteriorizing role of Wnt/beta-catenin signaling. Thus, DKKs inhibit Wnt coreceptor function, exemplifying the modulation of LRP signaling by antagonists

Full details of the abovementioned studies are described in the following publications, the disclosure of which are hereby incorporated by reference:
Krupnik, V. E.; Sharp, J. D.; Jiang, C.; Robison, K.; Chickering, T. W.; Amaravadi, L.; Brown, D. E.; Guyot, D.; Mays, G.; Leiby, K.; Chang, B.; Duong, T.; Goodearl, A. D. J.; Gearing, D. P.; Sokol, S. Y.; McCarthy, S. A.: Functional and structural diversity of the human Dickkopf gene family. Gene 238: 301-313, 1999.; and
Mao, B.; Wu, W.; Li, Y.; Hoppe, D.; Stannek, P.; Glinka, A.; Niehrs, C.: LDL-receptor-related protein 6 is a receptor for Dickkopf proteins. Nature 411: 321-325, 2001.

Further studies establishing the function and utilities of DKK1 are found in John Hopkins OMIM database record ID 605189, and in sited publications numbered 1137-113 and 1824 listed in the bibliography section hereinbelow, which are also hereby incorporated by reference. Keratocan (KERA, Accession NM_{—}007035) is another VGAM39 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM39 RNA, herein designated VGAM RNA, also designated SEQ ID:374.

Another function of VGAM39 is therefore inhibition of Keratocan (KERA, Accession NM_{—}007035), a gene which may be important in developing and maintaining corneal transparency and for the structure of the stromal matrix. Accordingly, utilities of VGAM39 include diagnosis, prevention and treatment of diseases and clinical conditions associated with KERA. The function of KERA has been established by previous studies. Keratan sulfate proteoglycans (KSPGs) are members of the small leucine-rich proteoglycan (SLRP) family. KSPGs, particularly keratocan, lumican (OMIM Ref. No. 600616), and mimecan (OMIM Ref. No. 602383), are important to the transparency of the cornea. Liu et al. (1998) isolated mouse keratocan cDNA and genomic DNA. Mouse keratocan cDNA predicts a 351-amino acid polypeptide containing a conserved central leucine-rich repeat region. Northern blot analysis of mouse tissues revealed that keratocan is expressed selectively in the eye throughout development. In situ hybridization demonstrated that keratocan is expressed early in neural crest development and later in corneal stromal cells. Tasheva et al. (1999) isolated the cDNA and identified the genomic structure of the human keratocan gene. The gene is spread over 7.65 kb of DNA and contains 3 exons. An open reading frame starting at the beginning of the second exon encodes a protein of 352 amino acids. The amino acid sequence of keratocan shows high identity among mammalian species. This evolutionary conservation between the keratocan proteins as well as the restricted expression of the KERA gene in cornea suggests that this molecule might be important in developing and maintaining corneal transparency.

Full details of the abovementioned studies are described in the following publications, the disclosure of which are hereby incorporated by reference:
Liu, C.-Y.; Shiraishi, A.; Kao, C. W.-C.; Converse, R. L.; Funderburgh, J. L.; Corpuz, L. M.; Conrad, G. W.; Kao, W. W.-Y.: The cloning of mouse keratocan cDNA and genomic DNA and the characterization of its expression during eye development. J. Biol. Chem. 273: 22584-22588, 1998.; and
Tasheva, E. S.; Funderburgh, J. L.; Funderburgh, M. L.; Corpuz, L. M.; Conrad, G. W.: Structure and sequence of the gene encoding human keratocan. DNA Seq. 10: 67-74, 1999.

Further studies establishing the function and utilities of KERA are found in John Hopkins OMIM database record ID 603288, and in sited publications numbered 1217, 2270, 84 and 1219-1220 listed in the bibliography section hereinbelow, which are also hereby incorporated by reference. Protein Phosphatase 1, Regulatory (inhibitor) Subunit 12A (PPP1R12A, Accession NM_{—}002480) is another VGAM39 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM39 RNA, herein designated VGAM RNA, also designated SEQ ID:374.

Another function of VGAM39 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM39 include diagnosis, prevention and treatment of diseases and clinical conditions associated with PPP1R12A. The function of PPP1R12A has been established by previous studies. Kimura et al. (1996) demonstrated that myosin phosphatase regulates the interaction of actin (see OMIM Ref. No. 102540) and myosin (see OMIM Ref. No. 160710) downstream of the guanosine triphosphatase Rho. Rho appears to inhibit myosin phosphatase through the action of Rho-kinase. Using the rat Mypt1 cDNA as probe, Takahashi et al. (1997) cloned a 4,855-bp cDNA for a human gene they symbolized MYPT1. Sequencing analysis showed that human MYPT1 contains 1,030 amino acids with a calculated molecular mass of approximately 115 kD. By fluorescence in situ hybridization, Kimura et al. (1996) mapped the MYPT1 gene to 12q15-q21.2. By radiation hybrid analysis, they showed that MYPT1 is located close to a highly polymorphic marker that lies between D12S350 and D12S106.

Full details of the abovementioned studies are described in the following publications, the disclosure of which are hereby incorporated by reference:
Kimura, K.; Ito, M.; Amano, M.; Chihara, K.; Fukata, Y.; Nakafuku, M.; Yamamori, B.; Feng, J.; Nakano, T.; Okawa, K.; Iwamatsu, A.; Kaibuchi, K.: Regulation of myosin phosphatase by Rho and Rho-associated kinase (Rho-kinase). Science 273: 245-248, 1996.; and
Takahashi, N.; Ito, M.; Tanaka, J.; Nakano, T.; Kaibuchi, K.; Odai, H.; Takemura, K.: Localization of the gene coding for myosin phosphatase, target subunit 1 (MYPT1) to human chromosome.

Further studies establishing the function and utilities of PPP1R12A are found in John Hopkins OMIM database record ID 602021, and in sited publications numbered 1342-1343 listed in the bibliography section hereinbelow, which are also hereby incorporated by reference. S100 Calcium Binding Protein, Beta (neural) (S100B, Accession NM_{—}006272) is another VGAM39 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM39 RNA, herein designated VGAM RNA, also designated SEQ ID:374.

Another function of VGAM39 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM39 include diagnosis, prevention and treatment of diseases and clinical conditions associated with S100B. The function of S100B has been established by previous studies. See 176940. The beta-subunit of S100 protein is expressed in glial cells at levels at least tenfold higher than in most other cell types. The brain also contains small amounts of the alpha subunits at levels approximately one-tenth that of the beta subunit. Allore et al. (1988) used genomic and cDNA probes in connection with a panel of rodent-human somatic cell hybrids to assign the S100B gene to 21q22. They suggested that this is a candidate gene for the neurologic disturbances in Down syndrome when present in trisomic state. By in situ hybridization, Duncan et al. (1989) localized the S100B gene to 21q22.2-q22.3. Allore et al. (1990) isolated overlapping genomic clones spanning the region coding for S100B and its flanking sequences. The intron/exon organization is similar to that of the genes coding for several other members of the S100 protein sub family. The S100B gene is composed of 3 exons, the first of which specifies the 5-prime untranslated region. Morii et al. (1991) isolated the S100A (OMIM Ref. No. 176940) and S100B genes from a human genomic DNA library. Endonuclease mapping and DNA sequencing showed that both comprise 3 exons and 2 introns. Two Ca(2+)-binding domains were independently encoded by exons 2 and 3. By spot-blot hybridization analysis of flow-sorted chromosomes, Morii et al. (1991) showed that the S100A and S100B genes are located on chromosome 1 and chromosome 21, respectively. Using restriction endonuclease fragment length variations (RFLV) in multipoint backcrosses, Shimizu et al. (1992) mapped the S100b gene in relation to other genes on mouse chromosome 10. The S100B gene is expressed at high levels in brain primarily by astrocytes. Addition of the disulfide-bonded dimeric form of the protein to primary neuronal and glial cultures and established cell lines induces axonal extension and alterations in astrocyte proliferation and phenotype. Reeves et al. (1994) demonstrated that the same effects of the S100B protein are exerted in vivo. They found that both astrocytosis and neurite proliferation occurred in transgenic mice expressing elevated levels of S100b. They suggested that these transgenic mice represent a useful model for studies of the role of S100B in glial-neuronal interactions in normal development and function of the brain and for analyzing the significance of elevated levels of the protein in Down syndrome and Alzheimer disease.

Full details of the abovementioned studies are described in the following publications, the disclosure of which are hereby incorporated by reference:
Reeves, R. H.; Yao, J.; Crowley, M. R.; Buck, S.; Zhang, X.; Yarowsky, P.; Gearhart, J. D.; Hilt, D. C.: Astrocytosis and axonal proliferation in the hippocampus of S100b transgenic mice. Proc. Nat. Acad. Sci. 91: 5359-5363, 1994.; and
Shimizu, A.; Sakai, Y.; Ohno, K.; Masaki, S.; Kuwano, R.; Takahashi, Y.; Miyashita, N.; Watanabe, T.: A molecular genetic linkage map of mouse chromosome 10, including the Myb, S100b, P.

Further studies establishing the function and utilities of S100B are found in John Hopkins OMIM database record ID 176990, and in sited publications numbered 204-206, 297 and 627 listed in the bibliography section hereinbelow, which are also hereby incorporated by reference. DKFZP56410422 (Accession NM_{—}031435) is another VGAM39 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM39 RNA, herein designated VGAM RNA, also designated SEQ ID:374.

Another function of VGAM39 is therefore inhibition of DKFZP56410422 (Accession NM_{—}031435). Accordingly, utilities of VGAM39 include diagnosis, prevention and treatment of diseases and clinical conditions associated with DKFZP56410422. Echinoderm Microtubule Associated Protein Like 4 (EML4, Accession NM_{—}019063) is another VGAM39 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM39 RNA, herein designated VGAM RNA, also designated SEQ ID:374.

Another function of VGAM39 is therefore inhibition of Echinoderm Microtubule Associated Protein Like 4 (EML4, Accession NM_{—}019063). Accordingly, utilities of VGAM39 include diagnosis, prevention and treatment of diseases and clinical conditions associated with EML4. FLJ21657 (Accession NM_{—}022483) is another VGAM39 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM39 RNA, herein designated VGAM RNA, also designated SEQ ID:374.

Another function of VGAM39 is therefore inhibition of FLJ21657 (Accession NM_{—}022483). Accordingly, utilities of VGAM39 include diagnosis, prevention and treatment of diseases and clinical conditions associated with FLJ21657. KIAA1054 (Accession XM_{—}043493) is another VGAM39 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM39 RNA, herein designated VGAM RNA, also designated SEQ ID:374.

Another function of VGAM39 is therefore inhibition of KIAA1054 (Accession XM_{—}043493). Accordingly, utilities of VGAM39 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM39 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM39 RNA, herein designated VGAM RNA, also designated SEQ ID:374.

Another function of VGAM39 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM39 include diagnosis, prevention and treatment of diseases and clinical conditions associated with SEMA3E. LOC130507 (Accession XM_{—}059440) is another VGAM39 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM39 RNA, herein designated VGAM RNA, also designated SEQ ID:374.

Another function of VGAM39 is therefore inhibition of LOC130507 (Accession XM_{—}059440). Accordingly, utilities of VGAM39 include diagnosis, prevention and treatment of diseases and clinical conditions associated with LOC130507. LOC148145 (Accession XM_{—}097410) is another VGAM39 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM39 RNA, herein designated VGAM RNA, also designated SEQ ID:374.

Another function of VGAM39 is therefore inhibition of LOC148145 (Accession XM_{—}097410). Accordingly, utilities of VGAM39 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 40 (VGAM40) viral gene, which modulates expression of respective host target genes thereof, the function and utility of which host target genes is known in the art.

VGAM40 is a novel bioinformatically detected regulatory, non protein coding, viral micro RNA (miRNA) gene. The method by which VGAM40 was detected is described hereinabove with reference to FIGS. 1-8.

VGAM40 gene, herein designated VGAM GENE, is a viral gene contained in the genome of Vaccinia Virus. VGAM40 host target gene, herein designated VGAM HOST TARGET GENE, is a human gene contained in the human genome.

VGAM40 gene encodes a VGAM40 precursor RNA, herein designated VGAM PRECURSOR RNA. Similar to other miRNA genes, and unlike most ordinary genes, VGAM40 precursor RNA does not encode a protein. A nucleotide sequence identical or highly similar to the nucleotide sequence of VGAM40 precursor RNA is designated SEQ ID:26, and is provided hereinbelow with reference to the sequence listing part. Nucleotide sequence SEQ ID:26 is located at position 963 relative to the genome of Vaccinia Virus.

VGAM40 precursor RNA folds onto itself, forming VGAM40 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM40 folded precursor RNA into VGAM40 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM40 RNA is designated SEQ ID:375, and is provided hereinbelow with reference to the sequence listing part.

VGAM40 host target gene, herein designated VGAM HOST TARGET GENE, encodes a corresponding messenger RNA, VGAM40 host target RNA, herein designated VGAM HOST TARGET RNA. VGAM40 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM40 RNA, herein designated VGAM RNA, binds complementarily to one or more host target binding sites located in untranslated regions of VGAM40 host target RNA, herein designated VGAM HOST TARGET RNA. This complementary binding is due to the fact that the nucleotide sequence of VGAM40 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM40 RNA, herein designated VGAM RNA, may have a different number of host target binding sites in untranslated regions of a VGAM40 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM40 RNA, herein designated VGAM RNA, to host target binding sites on VGAM40 host target RNA, herein designated VGAM HOST TARGET RNA, such as BINDING SITE I, BINDING SITE II and BINDING SITE III, inhibits translation of VGAM40 host target RNA into VGAM40 host target protein, herein designated VGAM HOST TARGET PROTEIN. VGAM host target protein is therefore outlined by a broken line.

It is appreciated that VGAM40 host target gene, herein designated VGAM HOST TARGET GENE, in fact represents a plurality of VGAM40 host target genes. The mRNA of each one of this plurality of VGAM40 host target genes comprises one or more host target binding sites, each having a nucleotide sequence which is at least partly complementary to VGAM40 RNA, herein designated VGAM RNA, and which when bound by VGAM40 RNA causes inhibition of translation of respective one or more VGAM40 host target proteins.

It is further appreciated by one skilled in the art that the mode of translational inhibition illustrated by FIG. 1 with specific reference to translational inhibition exerted by VGAM40 gene, herein designated VGAM GENE, on one or more VGAM40 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM40 is inhibition of expression of host target genes, as part of a novel viral mechanism of attacking a host. Accordingly, utilities of VGAM40 include diagnosis, prevention and treatment of viral infection by Vaccinia Virus. Specific functions, and accordingly utilities, of VGAM40 correlate with, and may be deduced from, the identity of the host target genes which VGAM40 binds and inhibits, and the function of these host target genes, as elaborated hereinbelow.

Nucleotide sequences of the VGAM40 precursor RNA, herein designated VGAM PRECURSOR RNA, and of the ‘diced’ VGAM40 RNA, herein designated VGAM RNA, and a schematic representation of the secondary folding of VGAM40 folded precursor RNA, herein designated VGAM FOLDED PRECURSOR RNA, of VGAM40 are further described hereinbelow with reference to Table 1.

Nucleotide sequences of host target binding sites, such as BINDING SITE-I, BINDING SITE-II and BINDING SITE-III of FIG. 1, found on VGAM40 host target RNA, and schematic representation of the complementarity of each of these host target binding sites to VGAM40 RNA, herein designated VGAM RNA, are described hereinbelow with reference to Table 2.

As mentioned hereinabove with reference to FIG. 1, a function of VGAM40 gene, herein designated VGAM is inhibition of expression of VGAM40 target genes. It is appreciated that specific functions, and accordingly utilities, of VGAM40 correlate with, and may be deduced from, the identity of the target genes which VGAM40 binds and inhibits, and the function of these target genes, as elaborated hereinbelow.

Dickkopf Homolog 1 (Xenopus laevis) (DKK1, Accession NM_{—}012242) is a VGAM40 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM40 RNA, herein designated VGAM RNA, also designated SEQ ID:375.

A function of VGAM40 is therefore inhibition of Dickkopf Homolog 1 (Xenopus laevis) (DKK1, Accession NM_{—}012242), a gene which is a growth factor and inhibits Wnt signaling. Accordingly, utilities of VGAM40 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM40 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM40 RNA, herein designated VGAM RNA, also designated SEQ ID:375.

Another function of VGAM40 is therefore inhibition of Keratocan (KERA, Accession NM_{—}007035), a gene which may be important in developing and maintaining corneal transparency and for the structure of the stromal matrix. Accordingly, utilities of VGAM40 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM40 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM40 RNA, herein designated VGAM RNA, also designated SEQ ID:375.

Another function of VGAM40 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM40 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM40 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM40 RNA, herein designated VGAM RNA, also designated SEQ ID:375.

Another function of VGAM40 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM40 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM40 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM40 RNA, herein designated VGAM RNA, also designated SEQ ID:375.

Another function of VGAM40 is therefore inhibition of DKFZP56410422 (Accession NM_{—}031435). Accordingly, utilities of VGAM40 include diagnosis, prevention and treatment of diseases and clinical conditions associated with DKFZP56410422. Echinoderm Microtubule Associated Protein Like 4 (EML4, Accession NM_{—}019063) is another VGAM40 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM40 RNA, herein designated VGAM RNA, also designated SEQ ID:375.

Another function of VGAM40 is therefore inhibition of Echinoderm Microtubule Associated Protein Like 4 (EML4, Accession NM_{—}019063). Accordingly, utilities of VGAM40 include diagnosis, prevention and treatment of diseases and clinical conditions associated with EML4. FLJ21657 (Accession NM_{—}022483) is another VGAM40 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM40 RNA, herein designated VGAM RNA, also designated SEQ ID:375.

Another function of VGAM40 is therefore inhibition of FLJ21657 (Accession NM_{—}022483). Accordingly, utilities of VGAM40 include diagnosis, prevention and treatment of diseases and clinical conditions associated with FLJ21657. KIAA1054 (Accession XM_{—}043493) is another VGAM40 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM40 RNA, herein designated VGAM RNA, also designated SEQ ID:375.

Another function of VGAM40 is therefore inhibition of KIAA1054 (Accession XM_{—}043493). Accordingly, utilities of VGAM40 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM40 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM40 RNA, herein designated VGAM RNA, also designated SEQ ID:375.

Another function of VGAM40 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM40 include diagnosis, prevention and treatment of diseases and clinical conditions associated with SEMA3E. LOC130507 (Accession XM_{—}059440) is another VGAM40 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM40 RNA, herein designated VGAM RNA, also designated SEQ ID:375.

Another function of VGAM40 is therefore inhibition of LOC130507 (Accession XM_{—}059440). Accordingly, utilities of VGAM40 include diagnosis, prevention and treatment of diseases and clinical conditions associated with LOC130507. LOC148145 (Accession XM_{—}097410) is another VGAM40 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM40 RNA, herein designated VGAM RNA, also designated SEQ ID:375.

Another function of VGAM40 is therefore inhibition of LOC148145 (Accession XM_{—}097410). Accordingly, utilities of VGAM40 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 41 (VGAM41) viral gene, which modulates expression of respective host target genes thereof, the function and utility of which host target genes is known in the art.

VGAM41 is a novel bioinformatically detected regulatory, non protein coding, viral micro RNA (miRNA) gene. The method by which VGAM41 was detected is described hereinabove with reference to FIGS. 1-8.

VGAM41 gene, herein designated VGAM GENE, is a viral gene contained in the genome of Vaccinia Virus. VGAM41 host target gene, herein designated VGAM HOST TARGET GENE, is a human gene contained in the human genome.

VGAM41 gene encodes a VGAM41 precursor RNA, herein designated VGAM PRECURSOR RNA. Similar to other miRNA genes, and unlike most ordinary genes, VGAM41 precursor RNA does not encode a protein. A nucleotide sequence identical or highly similar to the nucleotide sequence of VGAM41 precursor RNA is designated SEQ ID:27, and is provided hereinbelow with reference to the sequence listing part. Nucleotide sequence SEQ ID:27 is located at position 963 relative to the genome of Vaccinia Virus.

VGAM41 precursor RNA folds onto itself, forming VGAM41 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM41 folded precursor RNA into VGAM41 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM41 RNA is designated SEQ ID:376, and is provided hereinbelow with reference to the sequence listing part.

VGAM41 host target gene, herein designated VGAM HOST TARGET GENE, encodes a corresponding messenger RNA, VGAM41 host target RNA, herein designated VGAM HOST TARGET RNA. VGAM41 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM41 RNA, herein designated VGAM RNA, binds complementarily to one or more host target binding sites located in untranslated regions of VGAM41 host target RNA, herein designated VGAM HOST TARGET RNA. This complementary binding is due to the fact that the nucleotide sequence of VGAM41 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM41 RNA, herein designated VGAM RNA, may have a different number of host target binding sites in untranslated regions of a VGAM41 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM41 RNA, herein designated VGAM RNA, to host target binding sites on VGAM41 host target RNA, herein designated VGAM HOST TARGET RNA, such as BINDING SITE I, BINDING SITE II and BINDING SITE III, inhibits translation of VGAM41 host target RNA into VGAM41 host target protein, herein designated VGAM HOST TARGET PROTEIN. VGAM host target protein is therefore outlined by a broken line.

It is appreciated that VGAM41 host target gene, herein designated VGAM HOST TARGET GENE, in fact represents a plurality of VGAM41 host target genes. The mRNA of each one of this plurality of VGAM41 host target genes comprises one or more host target binding sites, each having a nucleotide sequence which is at least partly complementary to VGAM41 RNA, herein designated VGAM RNA, and which when bound by VGAM41 RNA causes inhibition of translation of respective one or more VGAM41 host target proteins.

It is further appreciated by one skilled in the art that the mode of translational inhibition illustrated by FIG. 1 with specific reference to translational inhibition exerted by VGAM41 gene, herein designated VGAM GENE, on one or more VGAM41 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM41 is inhibition of expression of host target genes, as part of a novel viral mechanism of attacking a host. Accordingly, utilities of VGAM41 include diagnosis, prevention and treatment of viral infection by Vaccinia Virus. Specific functions, and accordingly utilities, of VGAM41 correlate with, and may be deduced from, the identity of the host target genes which VGAM41 binds and inhibits, and the function of these host target genes, as elaborated hereinbelow.

Nucleotide sequences of the VGAM41 precursor RNA, herein designated VGAM PRECURSOR RNA, and of the ‘diced’ VGAM41 RNA, herein designated VGAM RNA, and a schematic representation of the secondary folding of VGAM41 folded precursor RNA, herein designated VGAM FOLDED PRECURSOR RNA, of VGAM41 are further described hereinbelow with reference to Table 1.

Nucleotide sequences of host target binding sites, such as BINDING SITE-I, BINDING SITE-II and BINDING SITE-III of FIG. 1, found on VGAM41 host target RNA, and schematic representation of the complementarity of each of these host target binding sites to VGAM41 RNA, herein designated VGAM RNA, are described hereinbelow with reference to Table 2.

As mentioned hereinabove with reference to FIG. 1, a function of VGAM41 gene, herein designated VGAM is inhibition of expression of VGAM41 target genes. It is appreciated that specific functions, and accordingly utilities, of VGAM41 correlate with, and may be deduced from, the identity of the target genes which VGAM41 binds and inhibits, and the function of these target genes, as elaborated hereinbelow.

Dickkopf Homolog 1 (Xenopus laevis) (DKK1, Accession NM_{—}012242) is a VGAM41 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM41 RNA, herein designated VGAM RNA, also designated SEQ ID:376.

A function of VGAM41 is therefore inhibition of Dickkopf Homolog 1 (Xenopus laevis) (DKK1, Accession NM_{—}012242), a gene which is a growth factor and inhibits Wnt signaling. Accordingly, utilities of VGAM41 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM41 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM41 RNA, herein designated VGAM RNA, also designated SEQ ID:376.

Another function of VGAM41 is therefore inhibition of Keratocan (KERA, Accession NM_{—}007035), a gene which may be important in developing and maintaining corneal transparency and for the structure of the stromal matrix. Accordingly, utilities of VGAM41 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM41 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM41 RNA, herein designated VGAM RNA, also designated SEQ ID:376.

Another function of VGAM41 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM41 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM41 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM41 RNA, herein designated VGAM RNA, also designated SEQ ID:376.

Another function of VGAM41 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM41 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM41 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM41 RNA, herein designated VGAM RNA, also designated SEQ ID:376.

Another function of VGAM41 is therefore inhibition of DKFZP56410422 (Accession NM_{—}031435). Accordingly, utilities of VGAM41 include diagnosis, prevention and treatment of diseases and clinical conditions associated with DKFZP56410422. Echinoderm Microtubule Associated Protein Like 4 (EML4, Accession NM_{—}019063) is another VGAM41 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM41 RNA, herein designated VGAM RNA, also designated SEQ ID:376.

Another function of VGAM41 is therefore inhibition of Echinoderm Microtubule Associated Protein Like 4 (EML4, Accession NM_{—}019063). Accordingly, utilities of VGAM41 include diagnosis, prevention and treatment of diseases and clinical conditions associated with EML4. FLJ21657 (Accession NM_{—}022483) is another VGAM41 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM41 RNA, herein designated VGAM RNA, also designated SEQ ID:376.

Another function of VGAM41 is therefore inhibition of FLJ21657 (Accession NM_{—}022483). Accordingly, utilities of VGAM41 include diagnosis, prevention and treatment of diseases and clinical conditions associated with FLJ21657. KIAA1054 (Accession XM_{—}043493) is another VGAM41 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM41 RNA, herein designated VGAM RNA, also designated SEQ ID:376.

Another function of VGAM41 is therefore inhibition of KIAA1054 (Accession XM_{—}043493). Accordingly, utilities of VGAM41 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM41 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM41 RNA, herein designated VGAM RNA, also designated SEQ ID:376.

Another function of VGAM41 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM41 include diagnosis, prevention and treatment of diseases and clinical conditions associated with SEMA3E. LOC130507 (Accession XM_{—}059440) is another VGAM41 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM41 RNA, herein designated VGAM RNA, also designated SEQ ID:376.

Another function of VGAM41 is therefore inhibition of LOC130507 (Accession XM_{—}059440). Accordingly, utilities of VGAM41 include diagnosis, prevention and treatment of diseases and clinical conditions associated with LOC130507. LOC148145 (Accession XM_{—}097410) is another VGAM41 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM41 RNA, herein designated VGAM RNA, also designated SEQ ID:376.

Another function of VGAM41 is therefore inhibition of LOC148145 (Accession XM_{—}097410). Accordingly, utilities of VGAM41 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 42 (VGAM42) viral gene, which modulates expression of respective host target genes thereof, the function and utility of which host target genes is known in the art.

VGAM42 is a novel bioinformatically detected regulatory, non protein coding, viral micro RNA (miRNA) gene. The method by which VGAM42 was detected is described hereinabove with reference to FIGS. 1-8.

VGAM42 gene, herein designated VGAM GENE, is a viral gene contained in the genome of Vaccinia Virus. VGAM42 host target gene, herein designated VGAM HOST TARGET GENE, is a human gene contained in the human genome.

VGAM42 gene encodes a VGAM42 precursor RNA, herein designated VGAM PRECURSOR RNA. Similar to other miRNA genes, and unlike most ordinary genes, VGAM42 precursor RNA does not encode a protein. A nucleotide sequence identical or highly similar to the nucleotide sequence of VGAM42 precursor RNA is designated SEQ ID:28, and is provided hereinbelow with reference to the sequence listing part. Nucleotide sequence SEQ ID:28 is located at position 963 relative to the genome of Vaccinia Virus.

VGAM42 precursor RNA folds onto itself, forming VGAM42 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM42 folded precursor RNA into VGAM42 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM42 RNA is designated SEQ ID:377, and is provided hereinbelow with reference to the sequence listing part.

VGAM42 host target gene, herein designated VGAM HOST TARGET GENE, encodes a corresponding messenger RNA, VGAM42 host target RNA, herein designated VGAM HOST TARGET RNA. VGAM42 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM42 RNA, herein designated VGAM RNA, binds complementarily to one or more host target binding sites located in untranslated regions of VGAM42 host target RNA, herein designated VGAM HOST TARGET RNA. This complementary binding is due to the fact that the nucleotide sequence of VGAM42 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM42 RNA, herein designated VGAM RNA, may have a different number of host target binding sites in untranslated regions of a VGAM42 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM42 RNA, herein designated VGAM RNA, to host target binding sites on VGAM42 host target RNA, herein designated VGAM HOST TARGET RNA, such as BINDING SITE I, BINDING SITE II and BINDING SITE III, inhibits translation of VGAM42 host target RNA into VGAM42 host target protein, herein designated VGAM HOST TARGET PROTEIN. VGAM host target protein is therefore outlined by a broken line.

It is appreciated that VGAM42 host target gene, herein designated VGAM HOST TARGET GENE, in fact represents a plurality of VGAM42 host target genes. The mRNA of each one of this plurality of VGAM42 host target genes comprises one or more host target binding sites, each having a nucleotide sequence which is at least partly complementary to VGAM42 RNA, herein designated VGAM RNA, and which when bound by VGAM42 RNA causes inhibition of translation of respective one or more VGAM42 host target proteins.

It is further appreciated by one skilled in the art that the mode of translational inhibition illustrated by FIG. 1 with specific reference to translational inhibition exerted by VGAM42 gene, herein designated VGAM GENE, on one or more VGAM42 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM42 is inhibition of expression of host target genes, as part of a novel viral mechanism of attacking a host. Accordingly, utilities of VGAM42 include diagnosis, prevention and treatment of viral infection by Vaccinia Virus. Specific functions, and accordingly utilities, of VGAM42 correlate with, and may be deduced from, the identity of the host target genes which VGAM42 binds and inhibits, and the function of these host target genes, as elaborated hereinbelow.

Nucleotide sequences of the VGAM42 precursor RNA, herein designated VGAM PRECURSOR RNA, and of the ‘diced’ VGAM42 RNA, herein designated VGAM RNA, and a schematic representation of the secondary folding of VGAM42 folded precursor RNA, herein designated VGAM FOLDED PRECURSOR RNA, of VGAM42 are further described hereinbelow with reference to Table 1.

Nucleotide sequences of host target binding sites, such as BINDING SITE-I, BINDING SITE-II and BINDING SITE-III of FIG. 1, found on VGAM42 host target RNA, and schematic representation of the complementarity of each of these host target binding sites to VGAM42 RNA, herein designated VGAM RNA, are described hereinbelow with reference to Table 2.

As mentioned hereinabove with reference to FIG. 1, a function of VGAM42 gene, herein designated VGAM is inhibition of expression of VGAM42 target genes. It is appreciated that specific functions, and accordingly utilities, of VGAM42 correlate with, and may be deduced from, the identity of the target genes which VGAM42 binds and inhibits, and the function of these target genes, as elaborated hereinbelow.

Dickkopf Homolog 1 (Xenopus laevis) (DKK1, Accession NM_{—}012242) is a VGAM42 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM42 RNA, herein designated VGAM RNA, also designated SEQ ID:377.

A function of VGAM42 is therefore inhibition of Dickkopf Homolog 1 (Xenopus laevis) (DKK1, Accession NM_{—}012242), a gene which is a growth factor and inhibits Wnt signaling. Accordingly, utilities of VGAM42 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM42 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM42 RNA, herein designated VGAM RNA, also designated SEQ ID:377.

Another function of VGAM42 is therefore inhibition of Keratocan (KERA, Accession NM_{—}007035), a gene which may be important in developing and maintaining corneal transparency and for the structure of the stromal matrix. Accordingly, utilities of VGAM42 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM42 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM42 RNA, herein designated VGAM RNA, also designated SEQ ID:377.

Another function of VGAM42 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM42 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM42 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM42 RNA, herein designated VGAM RNA, also designated SEQ ID:377.

Another function of VGAM42 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM42 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM42 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM42 RNA, herein designated VGAM RNA, also designated SEQ ID:377.

Another function of VGAM42 is therefore inhibition of DKFZP56410422 (Accession NM_{—}031435). Accordingly, utilities of VGAM42 include diagnosis, prevention and treatment of diseases and clinical conditions associated with DKFZP56410422. Echinoderm Microtubule Associated Protein Like 4 (EML4, Accession NM_{—}019063) is another VGAM42 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM42 RNA, herein designated VGAM RNA, also designated SEQ ID:377.

Another function of VGAM42 is therefore inhibition of Echinoderm Microtubule Associated Protein Like 4 (EML4, Accession NM_{—}019063). Accordingly, utilities of VGAM42 include diagnosis, prevention and treatment of diseases and clinical conditions associated with EML4. FLJ21657 (Accession NM_{—}022483) is another VGAM42 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM42 RNA, herein designated VGAM RNA, also designated SEQ ID:377.

Another function of VGAM42 is therefore inhibition of FLJ21657 (Accession NM_{—}022483). Accordingly, utilities of VGAM42 include diagnosis, prevention and treatment of diseases and clinical conditions associated with FLJ21657. KIAA1054 (Accession XM_{—}043493) is another VGAM42 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM42 RNA, herein designated VGAM RNA, also designated SEQ ID:377.

Another function of VGAM42 is therefore inhibition of KIAA1054 (Accession XM_{—}043493). Accordingly, utilities of VGAM42 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM42 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1431, to the nucleotide sequence of VGAM42 RNA, herein designated VGAM RNA, also designated SEQ ID:377.

Another function of VGAM42 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM42 include diagnosis, prevention and treatment of diseases and clinical conditions associated with SEMA3E. LOC130507 (Accession XM_{—}059440) is another VGAM42 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM42 RNA, herein designated VGAM RNA, also designated SEQ ID:377.

Another function of VGAM42 is therefore inhibition of LOC130507 (Accession XM_{—}059440). Accordingly, utilities of VGAM42 include diagnosis, prevention and treatment of diseases and clinical conditions associated with LOC130507. LOC148145 (Accession XM_{—}097410) is another VGAM42 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM42 RNA, herein designated VGAM RNA, also designated SEQ ID:377.

Another function of VGAM42 is therefore inhibition of LOC148145 (Accession XM_{—}097410). Accordingly, utilities of VGAM42 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 43 (VGAM43) viral gene, which modulates expression of respective host target genes thereof, the function and utility of which host target genes is known in the art.

VGAM43 is a novel bioinformatically detected regulatory, non protein coding, viral micro RNA (miRNA) gene. The method by which VGAM43 was detected is described hereinabove with reference to FIGS. 1-8.

VGAM43 gene, herein designated VGAM GENE, is a viral gene contained in the genome of Vaccinia Virus. VGAM43 host target gene, herein designated VGAM HOST TARGET GENE, is a human gene contained in the human genome.

VGAM43 gene encodes a VGAM43 precursor RNA, herein designated VGAM PRECURSOR RNA. Similar to other miRNA genes, and unlike most ordinary genes, VGAM43 precursor RNA does not encode a protein. A nucleotide sequence identical or highly similar to the nucleotide sequence of VGAM43 precursor RNA is designated SEQ ID:29, and is provided hereinbelow with reference to the sequence listing part. Nucleotide sequence SEQ ID:29 is located at position 963 relative to the genome of Vaccinia Virus.

VGAM43 precursor RNA folds onto itself, forming VGAM43 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM43 folded precursor RNA into VGAM43 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM43 RNA is designated SEQ ID:378, and is provided hereinbelow with reference to the sequence listing part.

VGAM43 host target gene, herein designated VGAM HOST TARGET GENE, encodes a corresponding messenger RNA, VGAM43 host target RNA, herein designated VGAM HOST TARGET RNA. VGAM43 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM43 RNA, herein designated VGAM RNA, binds complementarily to one or more host target binding sites located in untranslated regions of VGAM43 host target RNA, herein designated VGAM HOST TARGET RNA. This complementary binding is due to the fact that the nucleotide sequence of VGAM43 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM43 RNA, herein designated VGAM RNA, may have a different number of host target binding sites in untranslated regions of a VGAM43 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM43 RNA, herein designated VGAM RNA, to host target binding sites on VGAM43 host target RNA, herein designated VGAM HOST TARGET RNA, such as BINDING SITE I, BINDING SITE II and BINDING SITE III, inhibits translation of VGAM43 host target RNA into VGAM43 host target protein, herein designated VGAM HOST TARGET PROTEIN. VGAM host target protein is therefore outlined by a broken line.

It is appreciated that VGAM43 host target gene, herein designated VGAM HOST TARGET GENE, in fact represents a plurality of VGAM43 host target genes. The mRNA of each one of this plurality of VGAM43 host target genes comprises one or more host target binding sites, each having a nucleotide sequence which is at least partly complementary to VGAM43 RNA, herein designated VGAM RNA, and which when bound by VGAM43 RNA causes inhibition of translation of respective one or more VGAM43 host target proteins.

It is further appreciated by one skilled in the art that the mode of translational inhibition illustrated by FIG. 1 with specific reference to translational inhibition exerted by VGAM43 gene, herein designated VGAM GENE, on one or more VGAM43 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM43 is inhibition of expression of host target genes, as part of a novel viral mechanism of attacking a host. Accordingly, utilities of VGAM43 include diagnosis, prevention and treatment of viral infection by Vaccinia Virus. Specific functions, and accordingly utilities, of VGAM43 correlate with, and may be deduced from, the identity of the host target genes which VGAM43 binds and inhibits, and the function of these host target genes, as elaborated hereinbelow.

Nucleotide sequences of the VGAM43 precursor RNA, herein designated VGAM PRECURSOR RNA, and of the ‘diced’ VGAM43 RNA, herein designated VGAM RNA, and a schematic representation of the secondary folding of VGAM43 folded precursor RNA, herein designated VGAM FOLDED PRECURSOR RNA, of VGAM43 are further described hereinbelow with reference to Table 1.

Nucleotide sequences of host target binding sites, such as BINDING SITE-I, BINDING SITE-II and BINDING SITE-III of FIG. 1, found on VGAM43 host target RNA, and schematic representation of the complementarity of each of these host target binding sites to VGAM43 RNA, herein designated VGAM RNA, are described hereinbelow with reference to Table 2.

As mentioned hereinabove with reference to FIG. 1, a function of VGAM43 gene, herein designated VGAM is inhibition of expression of VGAM43 target genes. It is appreciated that specific functions, and accordingly utilities, of VGAM43 correlate with, and may be deduced from, the identity of the target genes which VGAM43 binds and inhibits, and the function of these target genes, as elaborated hereinbelow.

Dickkopf Homolog 1 (Xenopus laevis) (DKK1, Accession NM_{—}012242) is a VGAM43 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM43 RNA, herein designated VGAM RNA, also designated SEQ ID:378.

A function of VGAM43 is therefore inhibition of Dickkopf Homolog 1 (Xenopus laevis) (DKK1, Accession NM_{—}012242), a gene which is a growth factor and inhibits Wnt signaling. Accordingly, utilities of VGAM43 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM43 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM43 RNA, herein designated VGAM RNA, also designated SEQ ID:378.

Another function of VGAM43 is therefore inhibition of Keratocan (KERA, Accession NM_{—}007035), a gene which may be important in developing and maintaining corneal transparency and for the structure of the stromal matrix. Accordingly, utilities of VGAM43 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM43 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM43 RNA, herein designated VGAM RNA, also designated SEQ ID:378.

Another function of VGAM43 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM43 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM43 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM43 RNA, herein designated VGAM RNA, also designated SEQ ID:378.

Another function of VGAM43 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM43 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM43 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM43 RNA, herein designated VGAM RNA, also designated SEQ ID:378.

Another function of VGAM43 is therefore inhibition of DKFZP56410422 (Accession NM_{—}031435). Accordingly, utilities of VGAM43 include diagnosis, prevention and treatment of diseases and clinical conditions associated with DKFZP56410422. Echinoderm Microtubule Associated Protein Like 4 (EML4, Accession NM_{—}019063) is another VGAM43 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM43 RNA, herein designated VGAM RNA, also designated SEQ ID:378.

Another function of VGAM43 is therefore inhibition of Echinoderm Microtubule Associated Protein Like 4 (EML4, Accession NM_{—}019063). Accordingly, utilities of VGAM43 include diagnosis, prevention and treatment of diseases and clinical conditions associated with EML4. FLJ21657 (Accession NM_{—}022483) is another VGAM43 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM43 RNA, herein designated VGAM RNA, also designated SEQ ID:378.

Another function of VGAM43 is therefore inhibition of FLJ21657 (Accession NM_{—}022483). Accordingly, utilities of VGAM43 include diagnosis, prevention and treatment of diseases and clinical conditions associated with FLJ21657. KIAA1054 (Accession XM_{—}043493) is another VGAM43 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM43 RNA, herein designated VGAM RNA, also designated SEQ ID:378.

Another function of VGAM43 is therefore inhibition of KIAA1054 (Accession XM_{—}043493). Accordingly, utilities of VGAM43 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM43 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1431, to the nucleotide sequence of VGAM43 RNA, herein designated VGAM RNA, also designated SEQ ID:378.

Another function of VGAM43 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM43 include diagnosis, prevention and treatment of diseases and clinical conditions associated with SEMA3E. LOC130507 (Accession XM_{—}059440) is another VGAM43 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM43 RNA, herein designated VGAM RNA, also designated SEQ ID:378.

Another function of VGAM43 is therefore inhibition of LOC130507 (Accession XM_{—}059440). Accordingly, utilities of VGAM43 include diagnosis, prevention and treatment of diseases and clinical conditions associated with LOC130507. LOC148145 (Accession XM_{—}097410) is another VGAM43 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM43 RNA, herein designated VGAM RNA, also designated SEQ ID:378.

Another function of VGAM43 is therefore inhibition of LOC148145 (Accession XM_{—}097410). Accordingly, utilities of VGAM43 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 44 (VGAM44) viral gene, which modulates expression of respective host target genes thereof, the function and utility of which host target genes is known in the art.

VGAM44 is a novel bioinformatically detected regulatory, non protein coding, viral micro RNA (miRNA) gene. The method by which VGAM44 was detected is described hereinabove with reference to FIGS. 1-8.

VGAM44 gene, herein designated VGAM GENE, is a viral gene contained in the genome of Vaccinia Virus. VGAM44 host target gene, herein designated VGAM HOST TARGET GENE, is a human gene contained in the human genome.

VGAM44 gene encodes a VGAM44 precursor RNA, herein designated VGAM PRECURSOR RNA. Similar to other miRNA genes, and unlike most ordinary genes, VGAM44 precursor RNA does not encode a protein. A nucleotide sequence identical or highly similar to the nucleotide sequence of VGAM44 precursor RNA is designated SEQ ID:30, and is provided hereinbelow with reference to the sequence listing part. Nucleotide sequence SEQ ID:30 is located at position 963 relative to the genome of Vaccinia Virus.

VGAM44 precursor RNA folds onto itself, forming VGAM44 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM44 folded precursor RNA into VGAM44 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM44 RNA is designated SEQ ID:379, and is provided hereinbelow with reference to the sequence listing part.

VGAM44 host target gene, herein designated VGAM HOST TARGET GENE, encodes a corresponding messenger RNA, VGAM44 host target RNA, herein designated VGAM HOST TARGET RNA. VGAM44 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM44 RNA, herein designated VGAM RNA, binds complementarily to one or more host target binding sites located in untranslated regions of VGAM44 host target RNA, herein designated VGAM HOST TARGET RNA. This complementary binding is due to the fact that the nucleotide sequence of VGAM44 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM44 RNA, herein designated VGAM RNA, may have a different number of host target binding sites in untranslated regions of a VGAM44 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM44 RNA, herein designated VGAM RNA, to host target binding sites on VGAM44 host target RNA, herein designated VGAM HOST TARGET RNA, such as BINDING SITE I, BINDING SITE II and BINDING SITE III, inhibits translation of VGAM44 host target RNA into VGAM44 host target protein, herein designated VGAM HOST TARGET PROTEIN. VGAM host target protein is therefore outlined by a broken line.

It is appreciated that VGAM44 host target gene, herein designated VGAM HOST TARGET GENE, in fact represents a plurality of VGAM44 host target genes. The mRNA of each one of this plurality of VGAM44 host target genes comprises one or more host target binding sites, each having a nucleotide sequence which is at least partly complementary to VGAM44 RNA, herein designated VGAM RNA, and which when bound by VGAM44 RNA causes inhibition of translation of respective one or more VGAM44 host target proteins.

It is further appreciated by one skilled in the art that the mode of translational inhibition illustrated by FIG. 1 with specific reference to translational inhibition exerted by VGAM44 gene, herein designated VGAM GENE, on one or more VGAM44 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM44 is inhibition of expression of host target genes, as part of a novel viral mechanism of attacking a host. Accordingly, utilities of VGAM44 include diagnosis, prevention and treatment of viral infection by Vaccinia Virus. Specific functions, and accordingly utilities, of VGAM44 correlate with, and may be deduced from, the identity of the host target genes which VGAM44 binds and inhibits, and the function of these host target genes, as elaborated hereinbelow.

Nucleotide sequences of the VGAM44 precursor RNA, herein designated VGAM PRECURSOR RNA, and of the ‘diced’ VGAM44 RNA, herein designated VGAM RNA, and a schematic representation of the secondary folding of VGAM44 folded precursor RNA, herein designated VGAM FOLDED PRECURSOR RNA, of VGAM44 are further described hereinbelow with reference to Table 1.

Nucleotide sequences of host target binding sites, such as BINDING SITE-I, BINDING SITE-II and BINDING SITE-III of FIG. 1, found on VGAM44 host target RNA, and schematic representation of the complementarity of each of these host target binding sites to VGAM44 RNA, herein designated VGAM RNA, are described hereinbelow with reference to Table 2.

As mentioned hereinabove with reference to FIG. 1, a function of VGAM44 gene, herein designated VGAM is inhibition of expression of VGAM44 target genes. It is appreciated that specific functions, and accordingly utilities, of VGAM44 correlate with, and may be deduced from, the identity of the target genes which VGAM44 binds and inhibits, and the function of these target genes, as elaborated hereinbelow.

Dickkopf Homolog 1 (Xenopus laevis) (DKK1, Accession NM_{—}012242) is a VGAM44 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM44 RNA, herein designated VGAM RNA, also designated SEQ ID:379.

A function of VGAM44 is therefore inhibition of Dickkopf Homolog 1 (Xenopus laevis) (DKK1, Accession NM_{—}012242), a gene which is a growth factor and inhibits Wnt signaling. Accordingly, utilities of VGAM44 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM44 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM44 RNA, herein designated VGAM RNA, also designated SEQ ID:379.

Another function of VGAM44 is therefore inhibition of Keratocan (KERA, Accession NM_{—}007035), a gene which may be important in developing and maintaining corneal transparency and for the structure of the stromal matrix. Accordingly, utilities of VGAM44 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM44 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM44 RNA, herein designated VGAM RNA, also designated SEQ ID:379.

Another function of VGAM44 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM44 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM44 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM44 RNA, herein designated VGAM RNA, also designated SEQ ID:379.

Another function of VGAM44 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM44 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM44 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM44 RNA, herein designated VGAM RNA, also designated SEQ ID:379.

Another function of VGAM44 is therefore inhibition of DKFZP56410422 (Accession NM_{—}031435). Accordingly, utilities of VGAM44 include diagnosis, prevention and treatment of diseases and clinical conditions associated with DKFZP56410422. Echinoderm Microtubule Associated Protein Like 4 (EML4, Accession NM_{—}019063) is another VGAM44 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM44 RNA, herein designated VGAM RNA, also designated SEQ ID:379.

Another function of VGAM44 is therefore inhibition of Echinoderm Microtubule Associated Protein Like 4 (EML4, Accession NM_{—}019063). Accordingly, utilities of VGAM44 include diagnosis, prevention and treatment of diseases and clinical conditions associated with EML4. FLJ21657 (Accession NM_{—}022483) is another VGAM44 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM44 RNA, herein designated VGAM RNA, also designated SEQ ID:379.

Another function of VGAM44 is therefore inhibition of FLJ21657 (Accession NM_{—}022483). Accordingly, utilities of VGAM44 include diagnosis, prevention and treatment of diseases and clinical conditions associated with FLJ21657. KIAA1054 (Accession XM_{—}043493) is another VGAM44 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM44 RNA, herein designated VGAM RNA, also designated SEQ ID:379.

Another function of VGAM44 is therefore inhibition of KIAA1054 (Accession XM_{—}043493). Accordingly, utilities of VGAM44 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM44 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM44 RNA, herein designated VGAM RNA, also designated SEQ ID:379.

Another function of VGAM44 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM44 include diagnosis, prevention and treatment of diseases and clinical conditions associated with SEMA3E. LOC130507 (Accession XM_{—}059440) is another VGAM44 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM44 RNA, herein designated VGAM RNA, also designated SEQ ID:379.

Another function of VGAM44 is therefore inhibition of LOC130507 (Accession XM_{—}059440). Accordingly, utilities of VGAM44 include diagnosis, prevention and treatment of diseases and clinical conditions associated with LOC130507. LOC148145 (Accession XM_{—}097410) is another VGAM44 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM44 RNA, herein designated VGAM RNA, also designated SEQ ID:379.

Another function of VGAM44 is therefore inhibition of LOC148145 (Accession XM_{—}097410). Accordingly, utilities of VGAM44 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 45 (VGAM45) viral gene, which modulates expression of respective host target genes thereof, the function and utility of which host target genes is known in the art.

VGAM45 is a novel bioinformatically detected regulatory, non protein coding, viral micro RNA (miRNA) gene. The method by which VGAM45 was detected is described hereinabove with reference to FIGS. 1-8.

VGAM45 gene, herein designated VGAM GENE, is a viral gene contained in the genome of Vaccinia Virus. VGAM45 host target gene, herein designated VGAM HOST TARGET GENE, is a human gene contained in the human genome.

VGAM45 gene encodes a VGAM45 precursor RNA, herein designated VGAM PRECURSOR RNA. Similar to other miRNA genes, and unlike most ordinary genes, VGAM45 precursor RNA does not encode a protein. A nucleotide sequence identical or highly similar to the nucleotide sequence of VGAM45 precursor RNA is designated SEQ ID:31, and is provided hereinbelow with reference to the sequence listing part. Nucleotide sequence SEQ ID:31 is located at position 2576 relative to the genome of Vaccinia Virus.

VGAM45 precursor RNA folds onto itself, forming VGAM45 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM45 folded precursor RNA into VGAM45 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM45 RNA is designated SEQ ID:380, and is provided hereinbelow with reference to the sequence listing part.

VGAM45 host target gene, herein designated VGAM HOST TARGET GENE, encodes a corresponding messenger RNA, VGAM45 host target RNA, herein designated VGAM HOST TARGET RNA. VGAM45 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM45 RNA, herein designated VGAM RNA, binds complementarily to one or more host target binding sites located in untranslated regions of VGAM45 host target RNA, herein designated VGAM HOST TARGET RNA. This complementary binding is due to the fact that the nucleotide sequence of VGAM45 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM45 RNA, herein designated VGAM RNA, may have a different number of host target binding sites in untranslated regions of a VGAM45 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM45 RNA, herein designated VGAM RNA, to host target binding sites on VGAM45 host target RNA, herein designated VGAM HOST TARGET RNA, such as BINDING SITE I, BINDING SITE II and BINDING SITE III, inhibits translation of VGAM45 host target RNA into VGAM45 host target protein, herein designated VGAM HOST TARGET PROTEIN. VGAM host target protein is therefore outlined by a broken line.

It is appreciated that VGAM45 host target gene, herein designated VGAM HOST TARGET GENE, in fact represents a plurality of VGAM45 host target genes. The mRNA of each one of this plurality of VGAM45 host target genes comprises one or more host target binding sites, each having a nucleotide sequence which is at least partly complementary to VGAM45 RNA, herein designated VGAM RNA, and which when bound by VGAM45 RNA causes inhibition of translation of respective one or more VGAM45 host target proteins.

It is further appreciated by one skilled in the art that the mode of translational inhibition illustrated by FIG. 1 with specific reference to translational inhibition exerted by VGAM45 gene, herein designated VGAM GENE, on one or more VGAM45 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM45 is inhibition of expression of host target genes, as part of a novel viral mechanism of attacking a host. Accordingly, utilities of VGAM45 include diagnosis, prevention and treatment of viral infection by Vaccinia Virus. Specific functions, and accordingly utilities, of VGAM45 correlate with, and may be deduced from, the identity of the host target genes which VGAM45 binds and inhibits, and the function of these host target genes, as elaborated hereinbelow.

Nucleotide sequences of the VGAM45 precursor RNA, herein designated VGAM PRECURSOR RNA, and of the ‘diced’ VGAM45 RNA, herein designated VGAM RNA, and a schematic representation of the secondary folding of VGAM45 folded precursor RNA, herein designated VGAM FOLDED PRECURSOR RNA, of VGAM45 are further described hereinbelow with reference to Table 1.

Nucleotide sequences of host target binding sites, such as BINDING SITE-I, BINDING SITE-II and BINDING SITE-III of FIG. 1, found on VGAM45 host target RNA, and schematic representation of the complementarity of each of these host target binding sites to VGAM45 RNA, herein designated VGAM RNA, are described hereinbelow with reference to Table 2.

As mentioned hereinabove with reference to FIG. 1, a function of VGAM45 gene, herein designated VGAM is inhibition of expression of VGAM45 target genes. It is appreciated that specific functions, and accordingly utilities, of VGAM45 correlate with, and may be deduced from, the identity of the target genes which VGAM45 binds and inhibits, and the function of these target genes, as elaborated hereinbelow.

Dickkopf Homolog 1 (Xenopus laevis) (DKK1, Accession NM_{—}012242) is a VGAM45 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM45 RNA, herein designated VGAM RNA, also designated SEQ ID:380.

A function of VGAM45 is therefore inhibition of Dickkopf Homolog 1 (Xenopus laevis) (DKK1, Accession NM_{—}012242), a gene which is a growth factor and inhibits Wnt signaling. Accordingly, utilities of VGAM45 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM45 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM45 RNA, herein designated VGAM RNA, also designated SEQ ID:380.

Another function of VGAM45 is therefore inhibition of Keratocan (KERA, Accession NM_{—}007035), a gene which may be important in developing and maintaining corneal transparency and for the structure of the stromal matrix. Accordingly, utilities of VGAM45 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM45 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM45 RNA, herein designated VGAM RNA, also designated SEQ ID:380.

Another function of VGAM45 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM45 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM45 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM45 RNA, herein designated VGAM RNA, also designated SEQ ID:380.

Another function of VGAM45 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM45 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM45 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM45 RNA, herein designated VGAM RNA, also designated SEQ ID:380.

Another function of VGAM45 is therefore inhibition of DKFZP56410422 (Accession NM_{—}031435). Accordingly, utilities of VGAM45 include diagnosis, prevention and treatment of diseases and clinical conditions associated with DKFZP56410422. Echinoderm Microtubule Associated Protein Like 4 (EML4, Accession NM_{—}019063) is another VGAM45 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM45 RNA, herein designated VGAM RNA, also designated SEQ ID:380.

Another function of VGAM45 is therefore inhibition of Echinoderm Microtubule Associated Protein Like 4 (EML4, Accession NM_{—}019063). Accordingly, utilities of VGAM45 include diagnosis, prevention and treatment of diseases and clinical conditions associated with EML4. FLJ21657 (Accession NM_{—}022483) is another VGAM45 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM45 RNA, herein designated VGAM RNA, also designated SEQ ID:380.

Another function of VGAM45 is therefore inhibition of FLJ21657 (Accession NM_{—}022483). Accordingly, utilities of VGAM45 include diagnosis, prevention and treatment of diseases and clinical conditions associated with FLJ21657. KIAA1054 (Accession XM_{—}043493) is another VGAM45 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM45 RNA, herein designated VGAM RNA, also designated SEQ ID:380.

Another function of VGAM45 is therefore inhibition of KIAA1054 (Accession XM_{—}043493). Accordingly, utilities of VGAM45 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM45 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1431, to the nucleotide sequence of VGAM45 RNA, herein designated VGAM RNA, also designated SEQ ID:380.

Another function of VGAM45 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM45 include diagnosis, prevention and treatment of diseases and clinical conditions associated with SEMA3E. LOC130507 (Accession XM_{—}059440) is another VGAM45 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM45 RNA, herein designated VGAM RNA, also designated SEQ ID:380.

Another function of VGAM45 is therefore inhibition of LOC130507 (Accession XM_{—}059440). Accordingly, utilities of VGAM45 include diagnosis, prevention and treatment of diseases and clinical conditions associated with LOC130507. LOC148145 (Accession XM_{—}097410) is another VGAM45 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM45 RNA, herein designated VGAM RNA, also designated SEQ ID:380.

Another function of VGAM45 is therefore inhibition of LOC148145 (Accession XM_{—}097410). Accordingly, utilities of VGAM45 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 46 (VGAM46) viral gene, which modulates expression of respective host target genes thereof, the function and utility of which host target genes is known in the art.

VGAM46 is a novel bioinformatically detected regulatory, non protein coding, viral micro RNA (miRNA) gene. The method by which VGAM46 was detected is described hereinabove with reference to FIGS. 1-8.

VGAM46 gene, herein designated VGAM GENE, is a viral gene contained in the genome of Vaccinia Virus. VGAM46 host target gene, herein designated VGAM HOST TARGET GENE, is a human gene contained in the human genome.

VGAM46 gene encodes a VGAM46 precursor RNA, herein designated VGAM PRECURSOR RNA. Similar to other miRNA genes, and unlike most ordinary genes, VGAM46 precursor RNA does not encode a protein. A nucleotide sequence identical or highly similar to the nucleotide sequence of VGAM46 precursor RNA is designated SEQ ID:32, and is provided hereinbelow with reference to the sequence listing part. Nucleotide sequence SEQ ID:32 is located at position 4213 relative to the genome of Vaccinia Virus.

VGAM46 precursor RNA folds onto itself, forming VGAM46 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM46 folded precursor RNA into VGAM46 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM46 RNA is designated SEQ ID:381, and is provided hereinbelow with reference to the sequence listing part.

VGAM46 host target gene, herein designated VGAM HOST TARGET GENE, encodes a corresponding messenger RNA, VGAM46 host target RNA, herein designated VGAM HOST TARGET RNA. VGAM46 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM46 RNA, herein designated VGAM RNA, binds complementarily to one or more host target binding sites located in untranslated regions of VGAM46 host target RNA, herein designated VGAM HOST TARGET RNA. This complementary binding is due to the fact that the nucleotide sequence of VGAM46 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM46 RNA, herein designated VGAM RNA, may have a different number of host target binding sites in untranslated regions of a VGAM46 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM46 RNA, herein designated VGAM RNA, to host target binding sites on VGAM46 host target RNA, herein designated VGAM HOST TARGET RNA, such as BINDING SITE I, BINDING SITE II and BINDING SITE III, inhibits translation of VGAM46 host target RNA into VGAM46 host target protein, herein designated VGAM HOST TARGET PROTEIN. VGAM host target protein is therefore outlined by a broken line.

It is appreciated that VGAM46 host target gene, herein designated VGAM HOST TARGET GENE, in fact represents a plurality of VGAM46 host target genes. The mRNA of each one of this plurality of VGAM46 host target genes comprises one or more host target binding sites, each having a nucleotide sequence which is at least partly complementary to VGAM46 RNA, herein designated VGAM RNA, and which when bound by VGAM46 RNA causes inhibition of translation of respective one or more VGAM46 host target proteins.

It is further appreciated by one skilled in the art that the mode of translational inhibition illustrated by FIG. 1 with specific reference to translational inhibition exerted by VGAM46 gene, herein designated VGAM GENE, on one or more VGAM46 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM46 is inhibition of expression of host target genes, as part of a novel viral mechanism of attacking a host. Accordingly, utilities of VGAM46 include diagnosis, prevention and treatment of viral infection by Vaccinia Virus. Specific functions, and accordingly utilities, of VGAM46 correlate with, and may be deduced from, the identity of the host target genes which VGAM46 binds and inhibits, and the function of these host target genes, as elaborated hereinbelow.

Nucleotide sequences of the VGAM46 precursor RNA, herein designated VGAM PRECURSOR RNA, and of the ‘diced’ VGAM46 RNA, herein designated VGAM RNA, and a schematic representation of the secondary folding of VGAM46 folded precursor RNA, herein designated VGAM FOLDED PRECURSOR RNA, of VGAM46 are further described hereinbelow with reference to Table 1.

Nucleotide sequences of host target binding sites, such as BINDING SITE-I, BINDING SITE-II and BINDING SITE-III of FIG. 1, found on VGAM46 host target RNA, and schematic representation of the complementarity of each of these host target binding sites to VGAM46 RNA, herein designated VGAM RNA, are described hereinbelow with reference to Table 2.

As mentioned hereinabove with reference to FIG. 1, a function of VGAM46 gene, herein designated VGAM is inhibition of expression of VGAM46 target genes. It is appreciated that specific functions, and accordingly utilities, of VGAM46 correlate with, and may be deduced from, the identity of the target genes which VGAM46 binds and inhibits, and the function of these target genes, as elaborated hereinbelow.

EFG1 (Accession XM_{—}170611) is a VGAM46 host target gene. EFG1 BINDING SITE is HOST TARGET binding site found in the 3′ untranslated region of mRNA encoded by EFG1, corresponding to a HOST TARGET binding site such as BINDING SITE I, BINDING SITE II or BINDING SITE III. Table 2 illustrates the complementarity of the nucleotide sequences of EFG1 BINDING SITE, designated SEQ ID:3661, to the nucleotide sequence of VGAM46 RNA, herein designated VGAM RNA, also designated SEQ ID:381.

A function of VGAM46 is therefore inhibition of EFG1 (Accession XM_{—}170611), a gene which promotes the gtp-dependent translocation of the nascent protein chain from the a-site to the p-site of the ribosome in the mitochondria. Accordingly, utilities of VGAM46 include diagnosis, prevention and treatment of diseases and clinical conditions associated with EFG1. The function of EFG1 has been established by previous studies. By EST database searching with rat Efg as probe, followed by PCR of a testis cDNA library, Gao et al. (2001) obtained cDNAs encoding mouse and human EFG1, which they called GFM. EFG1 encodes a deduced 751-amino acid protein that shares 84% and 89% sequence identity with rat Efg and mouse Gfm, respectively, and contains a conserved GTP-binding elongation factor signature and a GTP-binding domain composed of 3 motifs. Northern blot analysis revealed wide expression of 3.8- and 3.4-kb transcripts, abundant in heart, skeletal muscle, and testis, as well as testis-specific expression of a 2.9-kb transcript. Independently, Hammarsund et al. (2001) identified and characterized mitochondrial elongation factor-2 (EFG2; 606544) and used information contained in public databases to identify and clone the complete coding sequence of the human EFG1 gene on chromosome 3q25.

Full details of the abovementioned studies are described in the following publications, the disclosure of which are hereby incorporated by reference:
Gao, J.; Yu, L.; Zhang, P.; Jiang, J.; Chen, J.; Peng, J.; Wei, Y.; Zhao, S.: Cloning and characterization of human and mouse mitochondrial elongation factor G, GFM and Gfm, and mapping of GFM to human chromosome 3q25.1-q26.2. Genomics 74: 109-114, 2001.; and
Hammarsund, M.; Wilson, W.; Corcoran, M.; Merup, M.; Einhorn, S.; Grander, D.; Sangfelt, O.: Identification and characterization of two novel human mitochondrial elongation factor gene.

Further studies establishing the function and utilities of EFG1 are found in John Hopkins OMIM database record ID 606639, and in sited publications numbered 1414 listed in the bibliography section hereinbelow, which are also hereby incorporated by reference. Phytanoyl-CoA Hydroxylase (Refsum disease) (PHYH, Accession NM_{—}006214) is another VGAM46 host target gene. PHYH BINDING SITE is HOST TARGET binding site found in the 3′ untranslated region of mRNA encoded by PHYH, corresponding to a HOST TARGET binding site such as BINDING SITE I, BINDING SITE II or BINDING SITE III. Table 2 illustrates the complementarity of the nucleotide sequences of PHYH BINDING SITE, designated SEQ ID:1281, to the nucleotide sequence of VGAM46 RNA, herein designated VGAM RNA, also designated SEQ ID:381.

Another function of VGAM46 is therefore inhibition of Phytanoyl-CoA Hydroxylase (Refsum disease) (PHYH, Accession NM_{—}006214). Accordingly, utilities of VGAM46 include diagnosis, prevention and treatment of diseases and clinical conditions associated with PHYH. Solute Carrier Family 6 (neurotransmitter transporter, dopamine), Member 3 (SLC6A3, Accession NM_{—}001044) is another VGAM46 host target gene. SLC6A3 BINDING SITE is HOST TARGET binding site found in the 3′ untranslated region of mRNA encoded by SLC6A3, corresponding to a HOST TARGET binding site such as BINDING SITE I, BINDING SITE II or BINDING SITE III. Table 2 illustrates the complementarity of the nucleotide sequences of SLC6A3 BINDING SITE, designated SEQ ID:795, to the nucleotide sequence of VGAM46 RNA, herein designated VGAM RNA, also designated SEQ ID:381.

Another function of VGAM46 is therefore inhibition of Solute Carrier Family 6 (neurotransmitter transporter, dopamine), Member 3 (SLC6A3, Accession NM_{—}001044), a gene which terminates the action of dopamine by its high affinity sodium-dependent reuptake into presynaptic terminals. Accordingly, utilities of VGAM46 include diagnosis, prevention and treatment of diseases and clinical conditions associated with SLC6A3. The function of SLC6A3 has been established by previous studies. Giros et al. (1996) found that the disruption of the mouse dopamine transporter gene results in spontaneous hyperlocomotion despite major adaptive changes such as decreases in neurotransmitter and receptor levels. In homozygous mice, dopamine persisted at least 100 times longer in the extracellular space, providing a biochemical explanation of the hyperdopaminergic phenotype and demonstrating the critical role of the transporter in regulating neurotransmission. The authors noted that the dopamine transporter is an obligatory target of cocaine and amphetamine, as demonstrated by the fact that these psychostimulants had no effect on locomotor activity or dopamine release and uptake in mice lacking the transporter. Giros et al. (1996) stated that the DAT knockout mice should be an excellent tool for the study and development of drugs used in the management of dopaminergic dysfunction. There are similarities between the hyperdopaminergic phenotype of the knockout mice and some of the positive symptoms of schizophrenic patients. Specific blockade of the dopamine transporter with high-affinity inhibitors may be beneficial in illnesses such as Parkinson disease, where the effective levels of dopamine are markedly reduced.

Full details of the abovementioned studies are described in the following publications, the disclosure of which are hereby incorporated by reference:
Gainetdinov, R. R.; Wetsel, W. C.; Jones, S. R.; Levin, E. D.; Jaber, M.; Caron, M. G.: Role of serotonin in the paradoxical calming effect of psychostimulants on hyperactivity. Science 283: 397-401, 1999.; and
Giros, B.; Jaber, M.; Jones, S. R.; Wightman, R. M.; Caron, M. G.: Hyperlocomotion and indifference to cocaine and amphetamine in mice lacking the dopamine receptor. Nature 370: 606-612, 1.

Further studies establishing the function and utilities of SLC6A3 are found in John Hopkins OMIM database record ID 126455, and in sited publications numbered 458-466, 20 and 517-523 listed in the bibliography section hereinbelow, which are also hereby incorporated by reference. H2AV (Accession NM_{—}138635) is another VGAM46 host target gene. H2AV BINDING SITE is HOST TARGET binding site found in the 3′ untranslated region of mRNA encoded by H2AV, corresponding to a HOST TARGET binding site such as BINDING SITE I, BINDING SITE II or BINDING SITE III. Table 2 illustrates the complementarity of the nucleotide sequences of H2AV BINDING SITE, designated SEQ ID:2447, to the nucleotide sequence of VGAM46 RNA, herein designated VGAM RNA, also designated SEQ ID:381.

Another function of VGAM46 is therefore inhibition of H2AV (Accession NM_{—}138635). Accordingly, utilities of VGAM46 include diagnosis, prevention and treatment of diseases and clinical conditions associated with H2AV. HMP19 (Accession XM_{—}113455) is another VGAM46 host target gene. HMP19 BINDING SITE is HOST TARGET binding site found in the 3′ untranslated region of mRNA encoded by HMP19, corresponding to a HOST TARGET binding site such as BINDING SITE I, BINDING SITE II or BINDING SITE III. Table 2 illustrates the complementarity of the nucleotide sequences of HMP19 BINDING SITE, designated SEQ ID:3408, to the nucleotide sequence of VGAM46 RNA, herein designated VGAM RNA, also designated SEQ ID:381.

Another function of VGAM46 is therefore inhibition of HMP19 (Accession XM_{—}113455). Accordingly, utilities of VGAM46 include diagnosis, prevention and treatment of diseases and clinical conditions associated with HMP19. KIAA1726 (Accession XM_{—}040860) is another VGAM46 host target gene. KIAA1726 BINDING SITE is HOST TARGET binding site found in the 3′ untranslated region of mRNA encoded by KIAA1726, corresponding to a HOST TARGET binding site such as BINDING SITE I, BINDING SITE II or BINDING SITE III. Table 2 illustrates the complementarity of the nucleotide sequences of KIAA1726 BINDING SITE, designated SEQ ID:2786, to the nucleotide sequence of VGAM46 RNA, herein designated VGAM RNA, also designated SEQ ID:381.

Another function of VGAM46 is therefore inhibition of KIAA1726 (Accession XM_{—}040860). Accordingly, utilities of VGAM46 include diagnosis, prevention and treatment of diseases and clinical conditions associated with KIAA1726. FIG. 1 further provides a conceptual description of a novel bioinformatically detected viral gene of the present invention, referred to here as Viral Genomic Address Messenger 47 (VGAM47) viral gene, which modulates expression of respective host target genes thereof, the function and utility of which host target genes is known in the art.

VGAM47 is a novel bioinformatically detected regulatory, non protein coding, viral micro RNA (miRNA) gene. The method by which VGAM47 was detected is described hereinabove with reference to FIGS. 1-8.

VGAM47 gene, herein designated VGAM GENE, is a viral gene contained in the genome of Vaccinia Virus. VGAM47 host target gene, herein designated VGAM HOST TARGET GENE, is a human gene contained in the human genome.

VGAM47 gene encodes a VGAM47 precursor RNA, herein designated VGAM PRECURSOR RNA. Similar to other miRNA genes, and unlike most ordinary genes, VGAM47 precursor RNA does not encode a protein. A nucleotide sequence identical or highly similar to the nucleotide sequence of VGAM47 precursor RNA is designated SEQ ID:33, and is provided hereinbelow with reference to the sequence listing part. Nucleotide sequence SEQ ID:33 is located at position 3249 relative to the genome of Vaccinia Virus.

VGAM47 precursor RNA folds onto itself, forming VGAM47 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM47 folded precursor RNA into VGAM47 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM47 RNA is designated SEQ ID:382, and is provided hereinbelow with reference to the sequence listing part.

VGAM47 host target gene, herein designated VGAM HOST TARGET GENE, encodes a corresponding messenger RNA, VGAM47 host target RNA, herein designated VGAM HOST TARGET RNA. VGAM47 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM47 RNA, herein designated VGAM RNA, binds complementarily to one or more host target binding sites located in untranslated regions of VGAM47 host target RNA, herein designated VGAM HOST TARGET RNA. This complementary binding is due to the fact that the nucleotide sequence of VGAM47 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM47 RNA, herein designated VGAM RNA, may have a different number of host target binding sites in untranslated regions of a VGAM47 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM47 RNA, herein designated VGAM RNA, to host target binding sites on VGAM47 host target RNA, herein designated VGAM HOST TARGET RNA, such as BINDING SITE I, BINDING SITE II and BINDING SITE III, inhibits translation of VGAM47 host target RNA into VGAM47 host target protein, herein designated VGAM HOST TARGET PROTEIN. VGAM host target protein is therefore outlined by a broken line.

It is appreciated that VGAM47 host target gene, herein designated VGAM HOST TARGET GENE, in fact represents a plurality of VGAM47 host target genes. The mRNA of each one of this plurality of VGAM47 host target genes comprises one or more host target binding sites, each having a nucleotide sequence which is at least partly complementary to VGAM47 RNA, herein designated VGAM RNA, and which when bound by VGAM47 RNA causes inhibition of translation of respective one or more VGAM47 host target proteins.

It is further appreciated by one skilled in the art that the mode of translational inhibition illustrated by FIG. 1 with specific reference to translational inhibition exerted by VGAM47 gene, herein designated VGAM GENE, on one or more VGAM47 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM47 is inhibition of expression of host target genes, as part of a novel viral mechanism of attacking a host. Accordingly, utilities of VGAM47 include diagnosis, prevention and treatment of viral infection by Vaccinia Virus. Specific functions, and accordingly utilities, of VGAM47 correlate with, and may be deduced from, the identity of the host target genes which VGAM47 binds and inhibits, and the function of these host target genes, as elaborated hereinbelow.

Nucleotide sequences of the VGAM47 precursor RNA, herein designated VGAM PRECURSOR RNA, and of the ‘diced’ VGAM47 RNA, herein designated VGAM RNA, and a schematic representation of the secondary folding of VGAM47 folded precursor RNA, herein designated VGAM FOLDED PRECURSOR RNA, of VGAM47 are further described hereinbelow with reference to Table 1.

Nucleotide sequences of host target binding sites, such as BINDING SITE-I, BINDING SITE-II and BINDING SITE-III of FIG. 1, found on VGAM47 host target RNA, and schematic representation of the complementarity of each of these host target binding sites to VGAM47 RNA, herein designated VGAM RNA, are described hereinbelow with reference to Table 2.

As mentioned hereinabove with reference to FIG. 1, a function of VGAM47 gene, herein designated VGAM is inhibition of expression of VGAM47 target genes. It is appreciated that specific functions, and accordingly utilities, of VGAM47 correlate with, and may be deduced from, the identity of the target genes which VGAM47 binds and inhibits, and the function of these target genes, as elaborated hereinbelow.

SRY (sex determining region Y)-box 11 (SOX11, Accession NM_{—}003108) is a VGAM47 host target gene. SOX11 BINDING SITE is HOST TARGET binding site found in the 3′ untranslated region of mRNA encoded by SOX11, corresponding to a HOST TARGET binding site such as BINDING SITE I, BINDING SITE II or BINDING SITE III. Table 2 illustrates the complementarity of the nucleotide sequences of SOX11 BINDING SITE, designated SEQ ID:992, to the nucleotide sequence of VGAM47 RNA, herein designated VGAM RNA, also designated SEQ ID:382.

A function of VGAM47 is therefore inhibition of SRY (sex determining region Y)-box 11 (SOX11, Accession NM_{—}003108). Accordingly, utilities of VGAM47 include diagnosis, prevention and treatment of diseases and clinical conditions associated with SOX11. CAP350 (Accession NM_{—}014810) is another VGAM47 host target gene. CAP350 BINDING SITE is HOST TARGET binding site found in the 3′ untranslated region of mRNA encoded by CAP350, corresponding to a HOST TARGET binding site such as BINDING SITE I, BINDING SITE II or BINDING SITE III. Table 2 illustrates the complementarity of the nucleotide sequences of CAP350 BINDING SITE, designated SEQ ID:1554, to the nucleotide sequence of VGAM47 RNA, herein designated VGAM RNA, also designated SEQ ID:382.

Another function of VGAM47 is therefore inhibition of CAP350 (Accession NM_{—}014810). Accordingly, utilities of VGAM47 include diagnosis, prevention and treatment of diseases and clinical conditions associated with CAP350. DKFZP564A022 (Accession NM_{—}030954) is another VGAM47 host target gene. DKFZP564A022 BINDING SITE is HOST TARGET binding site found in the 3′ untranslated region of mRNA encoded by DKFZP564A022, corresponding to a HOST TARGET binding site such as BINDING SITE I, BINDING SITE II or BINDING SITE III. Table 2 illustrates the complementarity of the nucleotide sequences of DKFZP564A022 BINDING SITE, designated SEQ ID:2174, to the nucleotide sequence of VGAM47 RNA, herein designated VGAM RNA, also designated SEQ ID:382.

Another function of VGAM47 is therefore inhibition of DKFZP564A022 (Accession NM_{—}030954). Accordingly, utilities of VGAM47 include diagnosis, prevention and treatment of diseases and clinical conditions associated with DKFZP564A022. FLJ11730 (Accession NM_{—}022756) is another VGAM47 host target gene. FLJ11730 BINDING SITE is HOST TARGET binding site found in the 3′ untranslated region of mRNA encoded by FLJ11730, corresponding to a HOST TARGET binding site such as BINDING SITE I, BINDING SITE II or BINDING SITE III. Table 2 illustrates the complementarity of the nucleotide sequences of FLJ11730 BINDING SITE, designated SEQ ID:2005, to the nucleotide sequence of VGAM47 RNA, herein designated VGAM RNA, also designated SEQ ID:382.

Another function of VGAM47 is therefore inhibition of FLJ11730 (Accession NM_{—}022756). Accordingly, utilities of VGAM47 include diagnosis, prevention and treatment of diseases and clinical conditions associated with FLJ11730. FLJ20686 (Accession NM_{—}017925) is another VGAM47 host target gene. FLJ20686 BINDING SITE is HOST TARGET binding site found in the 5′ untranslated region of mRNA encoded by FLJ20686, corresponding to a HOST TARGET binding site such as BINDING SITE I, BINDING SITE II or BINDING SITE III. Table 2 illustrates the complementarity of the nucleotide sequences of FLJ20686 BINDING SITE, designated SEQ ID:1765, to the nucleotide sequence of VGAM47 RNA, herein designated VGAM RNA, also designated SEQ ID:382.

Another function of VGAM47 is therefore inhibition of FLJ20686 (Accession NM_{—}017925). Accordingly, utilities of VGAM47 include diagnosis, prevention and treatment of diseases and clinical conditions associated with FLJ20686. FRSB (Accession NM_{—}005687) is another VGAM47 host target gene. FRSB BINDING SITE is HOST TARGET binding site found in the 3′ untranslated region of mRNA encoded by FRSB, corresponding to a HOST TARGET binding site such as BINDING SITE I, BINDING SITE II or BINDING SITE III. Table 2 illustrates the complementarity of the nucleotide sequences of FRSB BINDING SITE, designated SEQ ID:1236, to the nucleotide sequence of VGAM47 RNA, herein designated VGAM RNA, also designated SEQ ID:382.

Another function of VGAM47 is therefore inhibition of FRSB (Accession NM_{—}005687). Accordingly, utilities of VGAM47 include diagnosis, prevention and treatment of diseases and clinical conditions associated with FRSB. KIAA1600 (Accession XM_{—}049351) is another VGAM47 host target gene. KIAA1600 BINDING SITE is HOST TARGET binding site found in the 3′ untranslated region of mRNA encoded by KIAA1600, corresponding to a HOST TARGET binding site such as BINDING SITE I, BINDING SITE II or BINDING SITE III. Table 2 illustrates the complementarity of the nucleotide sequences of KIAA1600 BINDING SITE, designated SEQ ID:2919, to the nucleotide sequence of VGAM47 RNA, herein designated VGAM RNA, also designated SEQ ID:382.

Another function of VGAM47 is therefore inhibition of KIAA1600 (Accession XM_{—}049351). Accordingly, utilities of VGAM47 include diagnosis, prevention and treatment of diseases and clinical conditions associated with KIAA1600. LOC124801 (Accession XM_{—}058850) is another VGAM47 host target gene. LOC124801 BINDING SITE is HOST TARGET binding site found in the 3′ untranslated region of mRNA encoded by LOC124801, corresponding to a HOST TARGET binding site such as BINDING SITE I, BINDING SITE II or BINDING SITE III. Table 2 illustrates the complementarity of the nucleotide sequences of LOC124801 BINDING SITE, designated SEQ ID:2996, to the nucleotide sequence of VGAM47 RNA, herein designated VGAM RNA, also designated SEQ ID:382.

Another function of VGAM47 is therefore inhibition of LOC124801 (Accession XM_{—}058850). Accordingly, utilities of VGAM47 include diagnosis, prevention and treatment of diseases and clinical conditions associated with LOC124801. LOC221035 (Accession XM_{—}167640) is another VGAM47 host target gene. LOC221035 BINDING SITE is HOST TARGET binding site found in the 3′ untranslated region of mRNA encoded by LOC221035, corresponding to a HOST TARGET binding site such as BINDING SITE I, BINDING SITE II or BINDING SITE III. Table 2 illustrates the complementarity of the nucleotide sequences of LOC221035 BINDING SITE, designated SEQ ID:3593, to the nucleotide sequence of VGAM47 RNA, herein designated VGAM RNA, also designated SEQ ID:382.

Another function of VGAM47 is therefore inhibition of LOC221035 (Accession XM_{—}167640). Accordingly, utilities of VGAM47 include diagnosis, prevention and treatment of diseases and clinical conditions associated with LOC221035. LOC51611 (Accession NM_{—}015958) is another VGAM47 host target gene. LOC51611 BINDING SITE is HOST TARGET binding site found in the 3′ untranslated region of mRNA encoded by LOC51611, corresponding to a HOST TARGET binding site such as BINDING SITE I, BINDING SITE II or BINDING SITE III. Table 2 illustrates the complementarity of the nucleotide sequences of LOC51611 BINDING SITE, designated SEQ ID:1653, to the nucleotide sequence of VGAM47 RNA, herein designated VGAM RNA, also designated SEQ ID:382.

Another function of VGAM47 is therefore inhibition of LOC51611 (Accession NM_{—}015958). Accordingly, utilities of VGAM47 include diagnosis, prevention and treatment of diseases and clinical conditions associated with LOC51611. FIG. 1 further provides a conceptual description of a novel bioinformatically detected viral gene of the present invention, referred to here as Viral Genomic Address Messenger 48 (VGAM48) viral gene, which modulates expression of respective host target genes thereof, the function and utility of which host target genes is known in the art.

VGAM48 is a novel bioinformatically detected regulatory, non protein coding, viral micro RNA (miRNA) gene. The method by which VGAM48 was detected is described hereinabove with reference to FIGS. 1-8.

VGAM48 gene, herein designated VGAM GENE, is a viral gene contained in the genome of Vaccinia Virus. VGAM48 host target gene, herein designated VGAM HOST TARGET GENE, is a human gene contained in the human genome.

VGAM48 gene encodes a VGAM48 precursor RNA, herein designated VGAM PRECURSOR RNA. Similar to other miRNA genes, and unlike most ordinary genes, VGAM48 precursor RNA does not encode a protein. A nucleotide sequence identical or highly similar to the nucleotide sequence of VGAM48 precursor RNA is designated SEQ ID:34, and is provided hereinbelow with reference to the sequence listing part. Nucleotide sequence SEQ ID:34 is located at position 3681 relative to the genome of Vaccinia Virus.

VGAM48 precursor RNA folds onto itself, forming VGAM48 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM48 folded precursor RNA into VGAM48 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM48 RNA is designated SEQ ID:383, and is provided hereinbelow with reference to the sequence listing part.

VGAM48 host target gene, herein designated VGAM HOST TARGET GENE, encodes a corresponding messenger RNA, VGAM48 host target RNA, herein designated VGAM HOST TARGET RNA. VGAM48 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM48 RNA, herein designated VGAM RNA, binds complementarily to one or more host target binding sites located in untranslated regions of VGAM48 host target RNA, herein designated VGAM HOST TARGET RNA. This complementary binding is due to the fact that the nucleotide sequence of VGAM48 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM48 RNA, herein designated VGAM RNA, may have a different number of host target binding sites in untranslated regions of a VGAM48 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM48 RNA, herein designated VGAM RNA, to host target binding sites on VGAM48 host target RNA, herein designated VGAM HOST TARGET RNA, such as BINDING SITE I, BINDING SITE II and BINDING SITE III, inhibits translation of VGAM48 host target RNA into VGAM48 host target protein, herein designated VGAM HOST TARGET PROTEIN. VGAM host target protein is therefore outlined by a broken line.

It is appreciated that VGAM48 host target gene, herein designated VGAM HOST TARGET GENE, in fact represents a plurality of VGAM48 host target genes. The mRNA of each one of this plurality of VGAM48 host target genes comprises one or more host target binding sites, each having a nucleotide sequence which is at least partly complementary to VGAM48 RNA, herein designated VGAM RNA, and which when bound by VGAM48 RNA causes inhibition of translation of respective one or more VGAM48 host target proteins.

It is further appreciated by one skilled in the art that the mode of translational inhibition illustrated by FIG. 1 with specific reference to translational inhibition exerted by VGAM48 gene, herein designated VGAM GENE, on one or more VGAM48 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM48 is inhibition of expression of host target genes, as part of a novel viral mechanism of attacking a host. Accordingly, utilities of VGAM48 include diagnosis, prevention and treatment of viral infection by Vaccinia Virus. Specific functions, and accordingly utilities, of VGAM48 correlate with, and may be deduced from, the identity of the host target genes which VGAM48 binds and inhibits, and the function of these host target genes, as elaborated hereinbelow.

Nucleotide sequences of the VGAM48 precursor RNA, herein designated VGAM PRECURSOR RNA, and of the ‘diced’ VGAM48 RNA, herein designated VGAM RNA, and a schematic representation of the secondary folding of VGAM48 folded precursor RNA, herein designated VGAM FOLDED PRECURSOR RNA, of VGAM48 are further described hereinbelow with reference to Table 1.

Nucleotide sequences of host target binding sites, such as BINDING SITE-I, BINDING SITE-II and BINDING SITE-III of FIG. 1, found on VGAM48 host target RNA, and schematic representation of the complementarity of each of these host target binding sites to VGAM48 RNA, herein designated VGAM RNA, are described hereinbelow with reference to Table 2.

As mentioned hereinabove with reference to FIG. 1, a function of VGAM48 gene, herein designated VGAM is inhibition of expression of VGAM48 target genes. It is appreciated that specific functions, and accordingly utilities, of VGAM48 correlate with, and may be deduced from, the identity of the target genes which VGAM48 binds and inhibits, and the function of these target genes, as elaborated hereinbelow.

AXIN1 Up-regulated 1 (AXUD1, Accession NM_{—}033027) is a VGAM48 host target gene. AXUD1 BINDING SITE is HOST TARGET binding site found in the 3′ untranslated region of mRNA encoded by AXUD1, corresponding to a HOST TARGET binding site such as BINDING SITE I, BINDING SITE II or BINDING SITE III. Table 2 illustrates the complementarity of the nucleotide sequences of AXUD1 BINDING SITE, designated SEQ ID:2309, to the nucleotide sequence of VGAM48 RNA, herein designated VGAM RNA, also designated SEQ ID:383.

A function of VGAM48 is therefore inhibition of AXIN1 Up-regulated 1 (AXUD1, Accession NM_{—}033027). Accordingly, utilities of VGAM48 include diagnosis, prevention and treatment of diseases and clinical conditions associated with AXUD1. Bassoon (presynaptic cytomatrix protein) (BSN, Accession NM_{—}003458) is another VGAM48 host target gene. BSN BINDING SITE is HOST TARGET binding site found in the 3′ untranslated region of mRNA encoded by BSN, corresponding to a HOST TARGET binding site such as BINDING SITE I, BINDING SITE II or BINDING SITE III. Table 2 illustrates the complementarity of the nucleotide sequences of BSN BINDING SITE, designated SEQ ID: 1024, to the nucleotide sequence of VGAM48 RNA, herein designated VGAM RNA, also designated SEQ ID:383.

Another function of VGAM48 is therefore inhibition of Bassoon (presynaptic cytomatrix protein) (BSN, Accession NM_{—}003458), a gene which may be involved in cytomatrix organization at the site of neurotransmitter release. Accordingly, utilities of VGAM48 include diagnosis, prevention and treatment of diseases and clinical conditions associated with BSN. The function of BSN has been established by previous studies. Both the presynaptic terminal and the postsynaptic compartment of neuronal synapses comprise a highly specialized cytoskeleton underlying the synaptic membranes. The presynaptic nerve terminal is the principal site of regulated neurotransmitter release. The active zone is the region of the presynaptic plasmalemma over which synaptic vesicles dock, fuse, and release neurotransmitter. Piccolo (PCLO; 604918), a 420-kD protein, is 1 component of the presynaptic cytomatrix. Tom Dieck et al. (1998) isolated a large (greater than 400 kD) protein in mouse that is also found in the presynaptic compartments of rat brain synapses. They designated the protein Bassoon because it, along with Piccolo, is part of the ensemble of presynaptic proteins that are involved in orchestrating events at the nerve terminal. Bassoon is found in axon terminals of hippocampal neurons where it is highly concentrated in the vicinity of the active zone. Piccolo has a similar distribution and colocalizes with Bassoon in cultured hippocampal cells. Tom Dieck et al. (1998) suggested that Bassoon may be involved in cytomatrix organization at the site of neurotransmitter release Multiple system atrophy (MSA) is a sporadic progressive neurodegenerative disease. By differential hybridization to high-density cDNA filters, Hashida et al. (1998) identified human frontal lobe cDNAs with altered expression patterns in MSA patients. One partial cDNA whose expression was elevated 2-fold in MSA cerebella encoded a protein that the authors designated ZNF231 (zinc finger protein-231). By screening additional libraries with the partial cDNA, they assembled a full-length ZNF231 cDNA. The predicted 3,926-amino acid protein contains 2 glycine-proline dipeptide repeats, a pair of homologous C8 double zinc finger motifs, a leucine zipper motif, an SH3 domain-binding motif, 2 nuclear targeting sequences, 2 glutamine-rich domains, and a histidine-rich domain. Northern blot analysis of rat tissues indicated that the ZNF231 gene was expressed as a 16-kb mRNA specifically in brain. By RT-PCR of human brain cell lines and tissue, Hashida et al. (1998) determined that ZNF231 was expressed in the cerebellum and in a neuroblastoma cell line, but not in the white matter. Ishikawa et al. (1997) recovered a ZNF231 cDNA, designated KIAA0434, as 1 of 78 brain cDNAs that may encode large proteins. Gundelfinger (1999) stated that ZNF231 is the human homolog of Bassoon

Full details of the abovementioned studies are described in the following publications, the disclosure of which are hereby incorporated by reference:
tom Dieck, S.; Sanmarti-Vila, L.; Langnaese, K.; Richter, K.; Kindler, S.; Soyke, A.; Wex, H.; Smalla, K.-H.; Kampf, U.; Franzer, J.-T.; Stumm, M.; Garner, C. C.; Gundelfinger, E. D.: Bassoon, a novel zinc-finger CAG/glutamine-repeat protein selectively localized at the active zone of presynaptic nerve terminals. J. Cell Biol. 142: 499-509, 1998.; and
Hashida, H.; Goto, J.; Zhao, N.; Takahashi, N.; Hirai, M.; Kanazawa, I.; Sakaki, Y.: Cloning and mapping of ZNF231, a novel brain-specific gene encoding neuronal double zinc finger prot.

Further studies establishing the function and utilities of BSN are found in John Hopkins OMIM database record ID 604020, and in sited publications numbered 1737-1738, 25 and 1748-1749 listed in the bibliography section hereinbelow, which are also hereby incorporated by reference. Dihydropyrimidinase-like 2 (DPYSL2, Accession NM_{—}001386) is another VGAM48 host target gene. DPYSL2 BINDING SITE is HOST TARGET binding site found in the 3′ untranslated region of mRNA encoded by DPYSL2, corresponding to a HOST TARGET binding site such as BINDING SITE I, BINDING SITE II or BINDING SITE III. Table 2 illustrates the complementarity of the nucleotide sequences of DPYSL2 BINDING SITE, designated SEQ ID:822, to the nucleotide sequence of VGAM48 RNA, herein designated VGAM RNA, also designated SEQ ID:383.

Another function of VGAM48 is therefore inhibition of Dihydropyrimidinase-like 2 (DPYSL2, Accession NM_{—}001386), a gene which is a member of the dihydropyrimidinase family. Accordingly, utilities of VGAM48 include diagnosis, prevention and treatment of diseases and clinical conditions associated with DPYSL2. The function of DPYSL2 has been established by previous studies. Hamajima et al. (1996) isolated a human cDNA encoding dihydropyrimidinase-like 2 (OMIM Ref. No. DPYSL2), called DRP2 by them, from a fetal brain cDNA library (see OMIM Ref. No. 222748). The DPYSL2 protein has 572 amino acids. Northern blot analysis detected a 4.9-kb DPYSL2 transcript in all tissues examined except liver. Hamajima et al. (1996) noted that 3 ESTs mapped to 8p21 by Koyama et al. (1995) correspond to a portion of the coding region of DPYSL2.

Full details of the abovementioned studies are described in the following publications, the disclosure of which are hereby incorporated by reference:
Hamajima, N.; Matsuda, K.; Sakata, S.; Tamaki, N.; Sasaki, M.; Nonaka, M.: A novel gene family defined by human dihydropyrimidinase and three related proteins with differential tissue distribution. Gene 180: 157-163, 1996.; and
Koyama, K.; Sudo, K.; Nakamura, Y.: Isolation of 115 human chromosome 8-specific expressed-sequence tags by exon amplification. Genomics 26: 245-253, 1995.

Further studies establishing the function and utilities of DPYSL2 are found in John Hopkins OMIM database record ID 602463, and in sited publications numbered 75 and 1441 listed in the bibliography section hereinbelow, which are also hereby incorporated by reference. Epithelial V-like Antigen 1 (EVA1, Accession NM_{—}005797) is another VGAM48 host target gene. EVA1 BINDING SITE is HOST TARGET binding site found in the 3′ untranslated region of mRNA encoded by EVA1, corresponding to a HOST TARGET binding site such as BINDING SITE I, BINDING SITE II or BINDING SITE III. Table 2 illustrates the complementarity of the nucleotide sequences of EVA1 BINDING SITE, designated SEQ ID:1250, to the nucleotide sequence of VGAM48 RNA, herein designated VGAM RNA, also designated SEQ ID:383.

Another function of VGAM48 is therefore inhibition of Epithelial V-like Antigen 1 (EVA1, Accession NM_{—}005797). Accordingly, utilities of VGAM48 include diagnosis, prevention and treatment of diseases and clinical conditions associated with EVA1. Parathymosin (PTMS, Accession NM_{—}002824) is another VGAM48 host target gene. PTMS BINDING SITE is HOST TARGET binding site found in the 3′ untranslated region of mRNA encoded by PTMS, corresponding to a HOST TARGET binding site such as BINDING SITE I, BINDING SITE II or BINDING SITE III. Table 2 illustrates the complementarity of the nucleotide sequences of PTMS BINDING SITE, designated SEQ ID:959, to the nucleotide sequence of VGAM48 RNA, herein designated VGAM RNA, also designated SEQ ID:383.

Another function of VGAM48 is therefore inhibition of Parathymosin (PTMS, Accession NM_{—}002824), a gene which is involved in the regulation of cellular immunity. Accordingly, utilities of VGAM48 include diagnosis, prevention and treatment of diseases and clinical conditions associated with PTMS. The function of PTMS has been established by previous studies. Parathymosin is a polypeptide similar in size and amino acid composition to prothymosin-alpha (OMIM Ref. No. 188390). It has a high content of dicarboxylic amino acids and a complete absence of aromatic and sulfur-containing amino acids. It has 101 amino acid residues as compared to 111 for prothymosin. Clinton et al. (1989) reported the isolation of a cDNA clone for human kidney parathymosin containing the complete coding region and extending into the 5-prime and 3-prime flanking sequences. The open reading frame contains 306 nucleotides, including the codon for the initiator methionine. Analysis of the 5-prime flanking sequence excluded the presence of a hydrophobic signal peptide in the translated sequence. This permitted the conclusion that parathymosin, like prothymosin-alpha, is synthesized without formation of a larger precursor polypeptide. Parathymosin and prothymosin show a reciprocal relationship: the highest levels of parathymosin and its mRNA are present in liver, kidney, and brain (with lowest levels in thymus and spleen), whereas prothymosin-alpha and its mRNA are present in highest concentrations in thymus and spleen (with lower levels in kidney, brain, and liver). By in situ hybridization of rat parathymosin cDNA to human metaphase chromosomes, Szabo et al. (1989) localized the gene for human parathymosin to 17q12-q22.

Full details of the abovementioned studies are described in the following publications, the disclosure of which are hereby incorporated by reference:
Clinton, M.; Frangou-Lazaridis, M.; Panneerselvam, C.; Horecker, B. L.: The sequence of human parathymosin deduced from a cloned human kidney cDNA. Biochem. Biophys. Res. Commun. 158: 855-862, 1989.; and
Szabo, P.; Clinton, M.; Macera, M.; Horecker, B. L.: Localization of the gene coding for parathymosin to chromosome 17 in humans. Cytogenet. Cell Genet. 50: 91-92, 1989.

Further studies establishing the function and utilities of PTMS are found in John Hopkins OMIM database record ID 168440, and in sited publications numbered 572-573 listed in the bibliography section hereinbelow, which are also hereby incorporated by reference. Selectin E (endothelial adhesion molecule 1) (SELE, Accession NM_{—}000450) is another VGAM48 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:748, to the nucleotide sequence of VGAM48 RNA, herein designated VGAM RNA, also designated SEQ ID:383.

Another function of VGAM48 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM48 include diagnosis, prevention and treatment of diseases and clinical conditions associated with SELE. The function of SELE and its association with various diseases and clinical conditions, has been established by previous studies, as described hereinabove with reference to VGAM15. Solute Carrier Family 7 (cationic amino acid transporter, y+ system), Member 6 (SLC7A6, Accession NM_{—}003983) is another VGAM48 host target gene. SLC7A6 BINDING SITE is HOST TARGET binding site found in the 3′ untranslated region of mRNA encoded by SLC7A6, corresponding to a HOST TARGET binding site such as BINDING SITE I, BINDING SITE II or BINDING SITE III. Table 2 illustrates the complementarity of the nucleotide sequences of SLC7A6 BINDING SITE, designated SEQ ID: 1076, to the nucleotide sequence of VGAM48 RNA, herein designated VGAM RNA, also designated SEQ ID:383.

Another function of VGAM48 is therefore inhibition of Solute Carrier Family 7 (cationic amino acid transporter, y+ system), Member 6 (SLC7A6, Accession NM_{—}003983), a gene which is involved in mediating amino acid transport. Accordingly, utilities of VGAM48 include diagnosis, prevention and treatment of diseases and clinical conditions associated with SLC7A6. The function of SLC7A6 has been established by previous studies. Using RT-PCR with degenerate primers to screen for amino acid transporters in opossum kidney, followed by searching EST databases, Torrents et al. (1998) obtained a cDNA encoding SLC7A6, which they called y(+)LAT2. SLC7A6 is identical to the KIAA0245 gene reported by Nagase et al. (1996). Sequence analysis predicted that SLC7A6 is a 515-amino acid, typical organic solute transporter protein with 12 transmembrane domains, 3 potential phosphorylation sites, and N- and C-terminal cytoplasmic segments. SLC7A6 shares 75% amino acid identity with the opossum sequence and y(+)LAT1 (SLC7A7; 603593). By RT-PCR analysis, Nagase et al. (1996) detected SLC7A6 expression in all tissues tested except liver; expression was weak in pancreas and highest in thymus.

Full details of the abovementioned studies are described in the following publications, the disclosure of which are hereby incorporated by reference:
Nagase, T.; Seki, N.; Ishikawa, K.; Ohira, M.; Kawarabayasi, Y.; Ohara, O.; Tanaka, A.; Kotani, H.; Miyajima, N.; Nomura, N.: Prediction of the coding sequences of unidentified human genes. VI. The coding sequences of 80 new genes (KIAA0201-KIAA0280) deduced by analysis of cDNA clones from cell line KG-1 and brain. DNA Res. 3: 321-329, 1996.; and
Torrents, D.; Estevez, R.; Pineda, M.; Fernandez, E.; Lloberas, J.; Shi, Y.-B.; Zorzano, A.; Palacin, M.: Identification and characterization of a membrane protein (y(+)L amino acid tr.

Further studies establishing the function and utilities of SLC7A6 are found in John Hopkins OMIM database record ID 605641, and in sited publications numbered 2121 listed in the bibliography section hereinbelow, which are also hereby incorporated by reference. Transforming Growth Factor, Beta Receptor II (70/80 kDa) (TGFBR2, Accession NM_{—}003242) is another VGAM48 host target gene. TGFBR2 BINDING SITE is HOST TARGET binding site found in the 5′ untranslated region of mRNA encoded by TGFBR2, corresponding to a HOST TARGET binding site such as BINDING SITE I, BINDING SITE II or BINDING SITE III. Table 2 illustrates the complementarity of the nucleotide sequences of TGFBR2 BINDING SITE, designated SEQ ID: 1003, to the nucleotide sequence of VGAM48 RNA, herein designated VGAM RNA, also designated SEQ ID:383.

Another function of VGAM48 is therefore inhibition of Transforming Growth Factor, Beta Receptor II (70/80 kDa) (TGFBR2, Accession NM_{—}003242). Accordingly, utilities of VGAM48 include diagnosis, prevention and treatment of diseases and clinical conditions associated with TGFBR2. DKFZP586N0721 (Accession NM_{—}015400) is another VGAM48 host target gene. DKFZP586N0721 BINDING SITE is HOST TARGET binding site found in the 5′ untranslated region of mRNA encoded by DKFZP586N0721, corresponding to a HOST TARGET binding site such as BINDING SITE I, BINDING SITE II or BINDING SITE III. Table 2 illustrates the complementarity of the nucleotide sequences of DKFZP586N0721 BINDING SITE, designated SEQ ID:1627, to the nucleotide sequence of VGAM48 RNA, herein designated VGAM RNA, also designated SEQ ID:383.

Another function of VGAM48 is therefore inhibition of DKFZP586N0721 (Accession NM_{—}015400). Accordingly, utilities of VGAM48 include diagnosis, prevention and treatment of diseases and clinical conditions associated with DKFZP586N0721. DKFZp761J139 (Accession NM_{—}032280) is another VGAM48 host target gene. DKFZp761J139 BINDING SITE is HOST TARGET binding site found in the 5′ untranslated region of mRNA encoded by DKFZp761J139, corresponding to a HOST TARGET binding site such as BINDING SITE I, BINDING SITE II or BINDING SITE III. Table 2 illustrates the complementarity of the nucleotide sequences of DKFZp761J139 BINDING SITE, designated SEQ ID:2242, to the nucleotide sequence of VGAM48 RNA, herein designated VGAM RNA, also designated SEQ ID:383.

Another function of VGAM48 is therefore inhibition of DKFZp761J139 (Accession NM_{—}032280). Accordingly, utilities of VGAM48 include diagnosis, prevention and treatment of diseases and clinical conditions associated with DKFZp761J139. FLJ10468 (Accession NM_{—}018101) is another VGAM48 host target gene. FLJ10468 BINDING SITE is HOST TARGET binding site found in the 3′ untranslated region of mRNA encoded by FLJ10468, corresponding to a HOST TARGET binding site such as BINDING SITE I, BINDING SITE II or BINDING SITE III. Table 2 illustrates the complementarity of the nucleotide sequences of FLJ10468 BINDING SITE, designated SEQ ID:1779, to the nucleotide sequence of VGAM48 RNA, herein designated VGAM RNA, also designated SEQ ID:383.

Another function of VGAM48 is therefore inhibition of FLJ10468 (Accession NM_{—}018101). Accordingly, utilities of VGAM48 include diagnosis, prevention and treatment of diseases and clinical conditions associated with FLJ10468. FLJ20485 (Accession NM_{—}019042) is another VGAM48 host target gene. FLJ20485 BINDING SITE is HOST TARGET binding site found in the 3′ untranslated region of mRNA encoded by FLJ20485, corresponding to a HOST TARGET binding site such as BINDING SITE I, BINDING SITE II or BINDING SITE III. Table 2 illustrates the complementarity of the nucleotide sequences of FLJ20485 BINDING SITE, designated SEQ ID:1874, to the nucleotide sequence of VGAM48 RNA, herein designated VGAM RNA, also designated SEQ ID:383.

Another function of VGAM48 is therefore inhibition of FLJ20485 (Accession NM_{—}019042). Accordingly, utilities of VGAM48 include diagnosis, prevention and treatment of diseases and clinical conditions associated with FLJ20485. H2B Histone Family, Member Q (H2BFQ, Accession XM_{—}001940) is another VGAM48 host target gene. H2BFQ BINDING SITE is HOST TARGET binding site found in the 3′ untranslated region of mRNA encoded by H2BFQ, corresponding to a HOST TARGET binding site such as BINDING SITE I, BINDING SITE II or BINDING SITE III. Table 2 illustrates the complementarity of the nucleotide sequences of H2BFQ BINDING SITE, designated SEQ ID:2523, to the nucleotide sequence of VGAM48 RNA, herein designated VGAM RNA, also designated SEQ ID:383.

Another function of VGAM48 is therefore inhibition of H2B Histone Family, Member Q (H2BFQ, Accession XM_{—}001940). Accordingly, utilities of VGAM48 include diagnosis, prevention and treatment of diseases and clinical conditions associated with H2BFQ. KIAA0077 (Accession XM_{—}040158) is another VGAM48 host target gene. KIAA0077 BINDING SITE is HOST TARGET binding site found in the 3′ untranslated region of mRNA encoded by KIAA0077, corresponding to a HOST TARGET binding site such as BINDING SITE I, BINDING SITE II or BINDING SITE III. Table 2 illustrates the complementarity of the nucleotide sequences of KIAA0077 BINDING SITE, designated SEQ ID:2774, to the nucleotide sequence of VGAM48 RNA, herein designated VGAM RNA, also designated SEQ ID:383.

Another function of VGAM48 is therefore inhibition of KIAA0077 (Accession XM_{—}040158). Accordingly, utilities of VGAM48 include diagnosis, prevention and treatment of diseases and clinical conditions associated with KIAA0077. KIAA0295 (Accession XM_{—}042833) is another VGAM48 host target gene. KIAA0295 BINDING SITE is HOST TARGET binding site found in the 3′ untranslated region of mRNA encoded by KIAA0295, corresponding to a HOST TARGET binding site such as BINDING SITE I, BINDING SITE II or BINDING SITE III. Table 2 illustrates the complementarity of the nucleotide sequences of KIAA0295 BINDING SITE, designated SEQ ID:2807, to the nucleotide sequence of VGAM48 RNA, herein designated VGAM RNA, also designated SEQ ID:383.

Another function of VGAM48 is therefore inhibition of KIAA0295 (Accession XM_{—}042833). Accordingly, utilities of VGAM48 include diagnosis, prevention and treatment of diseases and clinical conditions associated with KIAA0295. KIAA0828 (Accession XM_{—}088105) is another VGAM48 host target gene. KIAA0828 BINDING SITE is HOST TARGET binding site found in the 3′ untranslated region of mRNA encoded by KIAA0828, corresponding to a HOST TARGET binding site such as BINDING SITE I, BINDING SITE II or BINDING SITE III. Table 2 illustrates the complementarity of the nucleotide sequences of KIAA0828 BINDING SITE, designated SEQ ID:3195, to the nucleotide sequence of VGAM48 RNA, herein designated VGAM RNA, also designated SEQ ID:383.

Another function of VGAM48 is therefore inhibition of KIAA0828 (Accession XM_{—}088105). Accordingly, utilities of VGAM48 include diagnosis, prevention and treatment of diseases and clinical conditions associated with KIAA0828. KIAA1203 (Accession XM_{—}049683) is another VGAM48 host target gene. KIAA1203 BINDING SITE is HOST TARGET binding site found in the 3′ untranslated region of mRNA encoded by KIAA1203, corresponding to a HOST TARGET binding site such as BINDING SITE I, BINDING SITE II or BINDING SITE III. Table 2 illustrates the complementarity of the nucleotide sequences of KIAA1203 BINDING SITE, designated SEQ ID:2921, to the nucleotide sequence of VGAM48 RNA, herein designated VGAM RNA, also designated SEQ ID:383.

Another function of VGAM48 is therefore inhibition of KIAA1203 (Accession XM_{—}049683). Accordingly, utilities of VGAM48 include diagnosis, prevention and treatment of diseases and clinical conditions associated with KIAA1203. Oxysterol Binding Protein-like 8 (OSBPL8, Accession NM_{—}020841) is another VGAM48 host target gene. OSBPL8 BINDING SITE is HOST TARGET binding site found in the 3′ untranslated region of mRNA encoded by OSBPL8, corresponding to a HOST TARGET binding site such as BINDING SITE I, BINDING SITE II or BINDING SITE III. Table 2 illustrates the complementarity of the nucleotide sequences of OSBPL8 BINDING SITE, designated SEQ ID:1928, to the nucleotide sequence of VGAM48 RNA, herein designated VGAM RNA, also designated SEQ ID:383.

Another function of VGAM48 is therefore inhibition of Oxysterol Binding Protein-like 8 (OSBPL8, Accession NM_{—}020841). Accordingly, utilities of VGAM48 include diagnosis, prevention and treatment of diseases and clinical conditions associated with OSBPL8. p21(CDKN1A)-activated Kinase 6 (PAK6, Accession NM_{—}020168) is another VGAM48 host target gene. PAK6 BINDING SITE is HOST TARGET binding site found in the 3′ untranslated region of mRNA encoded by PAK6, corresponding to a HOST TARGET binding site such as BINDING SITE I, BINDING SITE II or BINDING SITE III. Table 2 illustrates the complementarity of the nucleotide sequences of PAK6 BINDING SITE, designated SEQ ID:1897, to the nucleotide sequence of VGAM48 RNA, herein designated VGAM RNA, also designated SEQ ID:383.

Another function of VGAM48 is therefore inhibition of p21(CDKN1A)-activated Kinase 6 (PAK6, Accession NM_{—}020168). Accordingly, utilities of VGAM48 include diagnosis, prevention and treatment of diseases and clinical conditions associated with PAK6. Polymerase (RNA) III (DNA directed) Polypeptide F, 39 KDa (POLR3F, Accession XM_{—}009639) is another VGAM48 host target gene. POLR3F BINDING SITE is HOST TARGET binding site found in the 3′ untranslated region of mRNA encoded by POLR3F, corresponding to a HOST TARGET binding site such as BINDING SITE I, BINDING SITE II or BINDING SITE III. Table 2 illustrates the complementarity of the nucleotide sequences of POLR3F BINDING SITE, designated SEQ ID:2547, to the nucleotide sequence of VGAM48 RNA, herein designated VGAM RNA, also designated SEQ ID:383.

Another function of VGAM48 is therefore inhibition of Polymerase (RNA) III (DNA directed) Polypeptide F, 39 KDa (POLR3F, Accession XM_{—}009639). Accordingly, utilities of VGAM48 include diagnosis, prevention and treatment of diseases and clinical conditions associated with POLR3F. Ring Finger Protein 11 (RNF11, Accession NM_{—}014372) is another VGAM48 host target gene. RNF11 BINDING SITE is HOST TARGET binding site found in the 3′ untranslated region of mRNA encoded by RNF11, corresponding to a HOST TARGET binding site such as BINDING SITE I, BINDING SITE II or BINDING SITE III. Table 2 illustrates the complementarity of the nucleotide sequences of RNF11 BINDING SITE, designated SEQ ID: 1495, to the nucleotide sequence of VGAM48 RNA, herein designated VGAM RNA, also designated SEQ ID:383.

Another function of VGAM48 is therefore inhibition of Ring Finger Protein 11 (RNF11, Accession NM_{—}014372). Accordingly, utilities of VGAM48 include diagnosis, prevention and treatment of diseases and clinical conditions associated with RNF11. Solute Carrier Family 16 (monocarboxylic acid transporters), Member 6 (SLC16A6, Accession NM_{—}004694) is another VGAM48 host target gene. SLC16A6 BINDING SITE is HOST TARGET binding site found in the 5′ untranslated region of mRNA encoded by SLC16A6, corresponding to a HOST TARGET binding site such as BINDING SITE I, BINDING SITE II or BINDING SITE III. Table 2 illustrates the complementarity of the nucleotide sequences of SLC16A6 BINDING SITE, designated SEQ ID:1143, to the nucleotide sequence of VGAM48 RNA, herein designated VGAM RNA, also designated SEQ ID:383.

Another function of VGAM48 is therefore inhibition of Solute Carrier Family 16 (monocarboxylic acid transporters), Member 6 (SLC16A6, Accession NM_{—}004694). Accordingly, utilities of VGAM48 include diagnosis, prevention and treatment of diseases and clinical conditions associated with SLC16A6. LOC144321 (Accession XM_{—}096578) is another VGAM48 host target gene. LOC144321 BINDING SITE is HOST TARGET binding site found in the 3′ untranslated region of mRNA encoded by LOC144321, corresponding to a HOST TARGET binding site such as BINDING SITE I, BINDING SITE II or BINDING SITE III. Table 2 illustrates the complementarity of the nucleotide sequences of LOC144321 BINDING SITE, designated SEQ ID:3267, to the nucleotide sequence of VGAM48 RNA, herein designated VGAM RNA, also designated SEQ ID:383.

Another function of VGAM48 is therefore inhibition of LOC144321 (Accession XM_{—}096578). Accordingly, utilities of VGAM48 include diagnosis, prevention and treatment of diseases and clinical conditions associated with LOC144321. LOC150951 (Accession XM_{—}097975) is another VGAM48 host target gene. LOC150951 BINDING SITE is HOST TARGET binding site found in the 5′ untranslated region of mRNA encoded by LOC150951, corresponding to a HOST TARGET binding site such as BINDING SITE I, BINDING SITE II or BINDING SITE III. Table 2 illustrates the complementarity of the nucleotide sequences of LOC150951 BINDING SITE, designated SEQ ID:3336, to the nucleotide sequence of VGAM48 RNA, herein designated VGAM RNA, also designated SEQ ID:383.

Another function of VGAM48 is therefore inhibition of LOC150951 (Accession XM_{—}097975). Accordingly, utilities of VGAM48 include diagnosis, prevention and treatment of diseases and clinical conditions associated with LOC150951. LOC199920 (Accession XM_{—}114056) is another VGAM48 host target gene. LOC199920 BINDING SITE is HOST TARGET binding site found in the 5′ untranslated region of mRNA encoded by LOC199920, corresponding to a HOST TARGET binding site such as BINDING SITE I, BINDING SITE II or BINDING SITE III. Table 2 illustrates the complementarity of the nucleotide sequences of LOC199920 BINDING SITE, designated SEQ ID:3435, to the nucleotide sequence of VGAM48 RNA, herein designated VGAM RNA, also designated SEQ ID:383.

Another function of VGAM48 is therefore inhibition of LOC199920 (Accession XM_{—}114056). Accordingly, utilities of VGAM48 include diagnosis, prevention and treatment of diseases and clinical conditions associated with LOC199920. LOC256435 (Accession XM_{—}170925) is another VGAM48 host target gene. LOC256435 BINDING SITE is HOST TARGET binding site found in the 3′ untranslated region of mRNA encoded by LOC256435, corresponding to a HOST TARGET binding site such as BINDING SITE I, BINDING SITE II or BINDING SITE III. Table 2 illustrates the complementarity of the nucleotide sequences of LOC256435 BINDING SITE, designated SEQ ID:3675, to the nucleotide sequence of VGAM48 RNA, herein designated VGAM RNA, also designated SEQ ID:383.

Another function of VGAM48 is therefore inhibition of LOC256435 (Accession XM_{—}170925). Accordingly, utilities of VGAM48 include diagnosis, prevention and treatment of diseases and clinical conditions associated with LOC256435. FIG. 1 further provides a conceptual description of a novel bioinformatically detected viral gene of the present invention, referred to here as Viral Genomic Address Messenger 49 (VGAM49) viral gene, which modulates expression of respective host target genes thereof, the function and utility of which host target genes is known in the art.

VGAM49 is a novel bioinformatically detected regulatory, non protein coding, viral micro RNA (miRNA) gene. The method by which VGAM49 was detected is described hereinabove with reference to FIGS. 1-8.

VGAM49 gene, herein designated VGAM GENE, is a viral gene contained in the genome of Vaccinia Virus. VGAM49 host target gene, herein designated VGAM HOST TARGET GENE, is a human gene contained in the human genome.

VGAM49 gene encodes a VGAM49 precursor RNA, herein designated VGAM PRECURSOR RNA. Similar to other miRNA genes, and unlike most ordinary genes, VGAM49 precursor RNA does not encode a protein. A nucleotide sequence identical or highly similar to the nucleotide sequence of VGAM49 precursor RNA is designated SEQ ID:35, and is provided hereinbelow with reference to the sequence listing part. Nucleotide sequence SEQ ID:35 is located at position 3573 relative to the genome of Vaccinia Virus.

VGAM49 precursor RNA folds onto itself, forming VGAM49 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM49 folded precursor RNA into VGAM49 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM49 RNA is designated SEQ ID:384, and is provided hereinbelow with reference to the sequence listing part.

VGAM49 host target gene, herein designated VGAM HOST TARGET GENE, encodes a corresponding messenger RNA, VGAM49 host target RNA, herein designated VGAM HOST TARGET RNA. VGAM49 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM49 RNA, herein designated VGAM RNA, binds complementarily to one or more host target binding sites located in untranslated regions of VGAM49 host target RNA, herein designated VGAM HOST TARGET RNA. This complementary binding is due to the fact that the nucleotide sequence of VGAM49 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM49 RNA, herein designated VGAM RNA, may have a different number of host target binding sites in untranslated regions of a VGAM49 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM49 RNA, herein designated VGAM RNA, to host target binding sites on VGAM49 host target RNA, herein designated VGAM HOST TARGET RNA, such as BINDING SITE I, BINDING SITE II and BINDING SITE III, inhibits translation of VGAM49 host target RNA into VGAM49 host target protein, herein designated VGAM HOST TARGET PROTEIN. VGAM host target protein is therefore outlined by a broken line.

It is appreciated that VGAM49 host target gene, herein designated VGAM HOST TARGET GENE, in fact represents a plurality of VGAM49 host target genes. The mRNA of each one of this plurality of VGAM49 host target genes comprises one or more host target binding sites, each having a nucleotide sequence which is at least partly complementary to VGAM49 RNA, herein designated VGAM RNA, and which when bound by VGAM49 RNA causes inhibition of translation of respective one or more VGAM49 host target proteins.

It is further appreciated by one skilled in the art that the mode of translational inhibition illustrated by FIG. 1 with specific reference to translational inhibition exerted by VGAM49 gene, herein designated VGAM GENE, on one or more VGAM49 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM49 is inhibition of expression of host target genes, as part of a novel viral mechanism of attacking a host. Accordingly, utilities of VGAM49 include diagnosis, prevention and treatment of viral infection by Vaccinia Virus. Specific functions, and accordingly utilities, of VGAM49 correlate with, and may be deduced from, the identity of the host target genes which VGAM49 binds and inhibits, and the function of these host target genes, as elaborated hereinbelow.

Nucleotide sequences of the VGAM49 precursor RNA, herein designated VGAM PRECURSOR RNA, and of the ‘diced’ VGAM49 RNA, herein designated VGAM RNA, and a schematic representation of the secondary folding of VGAM49 folded precursor RNA, herein designated VGAM FOLDED PRECURSOR RNA, of VGAM49 are further described hereinbelow with reference to Table 1.

Nucleotide sequences of host target binding sites, such as BINDING SITE-I, BINDING SITE-II and BINDING SITE-III of FIG. 1, found on VGAM49 host target RNA, and schematic representation of the complementarity of each of these host target binding sites to VGAM49 RNA, herein designated VGAM RNA, are described hereinbelow with reference to Table 2.

As mentioned hereinabove with reference to FIG. 1, a function of VGAM49 gene, herein designated VGAM is inhibition of expression of VGAM49 target genes. It is appreciated that specific functions, and accordingly utilities, of VGAM49 correlate with, and may be deduced from, the identity of the target genes which VGAM49 binds and inhibits, and the function of these target genes, as elaborated hereinbelow.

A Disintegrin-like and Metalloprotease (reprolysin type) with Thrombospondin Type 1 Motif, 5 (aggrecanase-2) (ADAMTS5, Accession NM_{—}007038) is a VGAM49 host target gene. ADAMTS5 BINDING SITE is HOST TARGET binding site found in the 3′ untranslated region of mRNA encoded by ADAMTS5, corresponding to a HOST TARGET binding site such as BINDING SITE I, BINDING SITE II or BINDING SITE III. Table 2 illustrates the complementarity of the nucleotide sequences of ADAMTS5 BINDING SITE, designated SEQ ID:1352, to the nucleotide sequence of VGAM49 RNA, herein designated VGAM RNA, also designated SEQ ID:384.

A function of VGAM49 is therefore inhibition of A Disintegrin-like and Metalloprotease (reprolysin type) with Thrombospondin Type 1 Motif, 5 (aggrecanase-2) (ADAMTS5, Accession NM_{—}007038), a gene which cleaves aggrecan, a cartilage proteoglycan, and may be involved in its turnover. Accordingly, utilities of VGAM49 include diagnosis, prevention and treatment of diseases and clinical conditions associated with ADAMTS5. The function of ADAMTS5 has been established by previous studies. Proteolysis of the extracellular matrix plays a critical role in establishing tissue architecture during development and in tissue degradation in diseases such as cancer, arthritis, Alzheimer disease, and a variety of inflammatory conditions. The proteolytic enzymes responsible include members of diverse protease families and they may work in concert or in cascades to degrade or process molecules. Two groups of zinc metalloproteinases in particular, ADAMs and MMPs (e.g., 600754), appear broadly relevant to extracellular proteolysis. Most ADAM family members are quite similar in domain organization, bearing, from amino to carboxyl termini, a signal peptide, a proregion, a zinc metalloprotease catalytic domain with the typical reprolysin signature motif, a disintegrin domain, a cysteine-rich domain, an EGF-like domain, and, in many cases, a membrane-spanning region and a cytoplasmic domain with signaling potential. Members of the ADAMTS family differ substantially from the prototypic ADAM structure in that they lack the EGF-like domain, do not have a canonical disintegrin sequence, and possess modules with similar thrombospondin type 1 repeats. By searching an EST database using the protein sequences of human ADAMTS1 to ADAMTS4 and a C. elegans ADAMTS as queries, Hurskainen et al. (1999) identified ADAMTS5, ADAMTS6 (OMIM Ref. No. 605008), and ADAMTS7 (OMIM Ref. No. 605009). They determined a partial human ADAMTS5 cDNA sequence that lacked 5-prime coding sequence. The predicted partial ADAMTS5 protein has the domain structure characteristic of ADAMTSs, beginning with a partial metalloproteinase domain. Northern blot analysis of several human tissues detected an approximately 10-kb ADAMTS5 transcript that was expressed at a low level in placenta and at barely detectable levels in a number of other tissues. Northern blot analysis showed that mouse Adamts5 was specifically expressed in a 7-day mouse embryo, and at low or undetectable levels thereafter. In situ hybridization of an 8.5-day mouse embryo showed uniform Adamts5 expression throughout the embryo. In addition, Adamts5 expression was found in trophoblastic cells lining the uterine cavity, in the developing placenta, and in the decidual reaction within the uterus. In a 10.5-day mouse embryo, Adamts5 expression was widespread, but at a lower level than in the 8.5-day embryo. Expression was found in mesenchyme and somites, as well as in the neural tube and developing hindgut. Abbaszade et al. (1999) demonstrated that recombinant ADAMTS5 expressed in insect cells cleaves aggrecan at the glu373-ala374 site, with the cleavage pattern and inhibitor profile indistinguishable from that observed with native aggrecanase. Northern blot analysis of several human tissues showed highest ADAMTS5 expression in placenta, with much lower expression in heart and brain. Major transcripts of 12.4, 10.7, 8.6, and 6.6 kb were detected. Real time PCR of a number of normal human tissues detected ADAMTS5 expression in placenta, cervix, uterus, bladder, and esophagus. Expression was also found in rib cartilage, chondroblastoma, and fibrous tissue and joint capsule samples from an arthritic patient.

Full details of the abovementioned studies are described in the following publications, the disclosure of which are hereby incorporated by reference:
Abbaszade, I.; Liu, R.-Q.; Yang, F.; Rosenfeld, S. A.; Ross, O. H.; Link, J. R.; Ellis, D. M.; Tortorella, M. D.; Pratta, M. A.; Hollis, J. M.; Wynn, R.; Duke, J. L.; and 15 others: Cloning and characterization of ADAMTS11, an aggrecanase from the ADAMTS family. J. Biol. Chem. 274: 23443-23450, 1999.; and
Hurskainen, T. L.; Hirohata, S.; Seldin, M. F.; Apte, S. S.: ADAM-TS5, ADAM-TS6, and ADAM-TS7, novel members of a new family of zinc metalloproteases: general features and genomic dis.

Further studies establishing the function and utilities of ADAMTS5 are found in John Hopkins OMIM database record ID 605007, and in sited publications numbered 67 and 1735 listed in the bibliography section hereinbelow, which are also hereby incorporated by reference. Adenosine Deaminase, TRNA-specific 1 (ADAT1, Accession NM_{—}012091) is another VGAM49 host target gene. ADAT1 BINDING SITE is HOST TARGET binding site found in the 3′ untranslated region of mRNA encoded by ADAT1, corresponding to a HOST TARGET binding site such as BINDING SITE I, BINDING SITE II or BINDING SITE III. Table 2 illustrates the complementarity of the nucleotide sequences of ADAT1 BINDING SITE, designated SEQ ID: 1404, to the nucleotide sequence of VGAM49 RNA, herein designated VGAM RNA, also designated SEQ ID:384.

Another function of VGAM49 is therefore inhibition of Adenosine Deaminase, TRNA-specific 1 (ADAT1, Accession NM_{—}012091), a gene which TRNA-specific adenosine deaminase; deaminates A(37) in the anticodon loop of tRNA(Ala). Accordingly, utilities of VGAM49 include diagnosis, prevention and treatment of diseases and clinical conditions associated with ADAT1. The function of ADAT1 has been established by previous studies. The mammalian RNA-specific adenosine deaminases (ADARs; OMIM Ref. No. 601059) constitute a family of sequence-related proteins involved in pre-mRNA editing of nuclear transcripts through site-specific adenosine modification. Maas et al. (1999) identified and characterized a human ADAR-related protein that specifically deaminates adenosine-37 to inosine in eukaryotic tRNA(ala). They designated this predicted 502-amino acid protein ‘adenosine deaminase acting on tRNA,’ or ADAT1, and concluded that it probably represents the human counterpart of the yeast protein Tad1p. Southern blot analysis revealed that the ADAT1 enzyme is represented by a single gene. Northern blot analysis detected ADAT1 transcripts of approximately 5 and 6.5 kb in all human tissues, with highest expression levels in heart, brain, and pancreas. By radiation hybrid panel analysis, Maas et al. (2001) mapped the ADAT1 gene and the gene encoding lysyl tRNA synthetase (KARS; 601421) to 16q22.2-q22.3, with the gene for alanyl tRNA synthetase (AARS; 601065) positioned centromeric to the KARS and ADAT1 genes. They speculated that the clustering of 3 tRNA-specific genes, of which 2 are specific for tRNA(Ala), may indicate their evolutionary relatedness or common factors involved in regulating their expression.

Full details of the abovementioned studies are described in the following publications, the disclosure of which are hereby incorporated by reference:
Maas, S.; Gerber, A. P.; Rich, A.: Identification and characterization of a human tRNA-specific adenosine deaminase related to the ADAR family of pre-mRNA editing enzymes. Proc. Nat. Acad. Sci. 96: 8895-8900, 1999.; and
Maas, S.; Kim, Y.-G.; Rich, A.: Genomic clustering of tRNA-specific adenosine deaminase ADAT1 and two tRNA synthetases. Mammalian Genome 12: 387-393, 2001.

Further studies establishing the function and utilities of ADAT1 are found in John Hopkins OMIM database record ID 604230, and in sited publications numbered 118 and 1787 listed in the bibliography section hereinbelow, which are also hereby incorporated by reference. Adenylate Cyclase 2 (brain) (ADCY2, Accession XM_{—}036383) is another VGAM49 host target gene. ADCY2 BINDING SITE is HOST TARGET binding site found in the 3′ untranslated region of mRNA encoded by ADCY2, corresponding to a HOST TARGET binding site such as BINDING SITE I, BINDING SITE II or BINDING SITE III. Table 2 illustrates the complementarity of the nucleotide sequences of ADCY2 BINDING SITE, designated SEQ ID:2711, to the nucleotide sequence of VGAM49 RNA, herein designated VGAM RNA, also designated SEQ ID:384.

Another function of VGAM49 is therefore inhibition of Adenylate Cyclase 2 (brain) (ADCY2, Accession XM_{—}036383), a gene which Adenylate cyclase (type 2), an ATP-pyrophosphate lyase; converts ATP to cAMP. Accordingly, utilities of VGAM49 include diagnosis, prevention and treatment of diseases and clinical conditions associated with ADCY2. The function of ADCY2 has been established by previous studies. Stengel et al. (1992) identified a brain cDNA corresponding to a gene that encodes a human brain adenylyl cyclase, which they symbolized HBAC2. The amino acid sequence of ADCY2 displayed significant homology with ADCY8 (OMIM Ref. No. 103070) in the highly conserved adenylyl cyclase domain (250 amino acids) found in the 3-prime cytoplasmic portion of all mammalian adenylyl cyclases. However, outside this domain, the homology was extremely low. By in situ hybridization to metaphase chromosomal spreads using a human brain cDNA probe, they demonstrated that the ADCY2 gene maps to 5p15.3. There was no cross-reactivity with the site on 8q24.2 where ADCY8 was found to map. Using Southern blot analysis of somatic cell hybrid DNAs, Gaudin et al. (1994) likewise mapped type II adenylyl cyclase to chromosome 5. Furthermore, they determined the chromosomal location of 4 other isoforms: type III on chromosome 2, type IV on chromosome 14, type V on chromosome 3, and type VI on chromosome 12. By fluorescence in situ hybridization, Edelhoff et al. (1995) mapped the mouse homolog to chromosome 13 in the C1 region. Wong et al. (2000) identified the presence of adenylyl cyclases 2, 3 (OMIM Ref. No. 600291), and 4 (OMIM Ref. No. 600292) in olfactory cilia.

Full details of the abovementioned studies are described in the following publications, the disclosure of which are hereby incorporated by reference:
Edelhoff, S.; Villacres, E. C.; Storm, D. R.; Disteche, C. M.: Mapping of adenylyl cyclase genes type I, II, III, IV, V, and VI in mouse. Mammalian Genome 6: 111-113, 1995.; and
Wong, S. T.; Trinh, K.; Hacker, B.; Chan, G. C. K.; Lowe, G.; Gaggar, A.; Xia, Z.; Gold, G. H.; Storm, D. R.: Disruption of the type III adenylyl cyclase gene leads to peripheral and be.

Further studies establishing the function and utilities of ADCY2 are found in John Hopkins OMIM database record ID 103071, and in sited publications numbered 85-86, 8 and 87 listed in the bibliography section hereinbelow, which are also hereby incorporated by reference. Aryl Hydrocarbon Receptor (AHR, Accession NM_{—}001621) is another VGAM49 host target gene. AHR BINDING SITE is HOST TARGET binding site found in the 3′ untranslated region of mRNA encoded by AHR, corresponding to a HOST TARGET binding site such as BINDING SITE I, BINDING SITE II or BINDING SITE III. Table 2 illustrates the complementarity of the nucleotide sequences of AHR BINDING SITE, designated SEQ ID:840, to the nucleotide sequence of VGAM49 RNA, herein designated VGAM RNA, also designated SEQ ID:384.

Another function of VGAM49 is therefore inhibition of Aryl Hydrocarbon Receptor (AHR, Accession NM_{—}001621), a gene which plays a role in modulating carcinogenesis through the induction of xenobiotic-metabolizing enzymes. Accordingly, utilities of VGAM49 include diagnosis, prevention and treatment of diseases and clinical conditions associated with AHR. The function of AHR has been established by previous studies. Halogenated aromatic hydrocarbons, represented by 2,3,7,8-tetrachlorodibenzo-p-dioxin (TCDD), are environmental pollutants that are produced by minor side-reactions in chemical manufacturing processes and by combustion of waste materials. These chemicals cause potent and pleiotropic toxicity, including teratogenesis, immune suppression, epithelial disorders, and tumor production in experimental animals. At the molecular level, aldehyde dehydrogenase, quinone reductase, and various drug-metabolizing enzymes are induced by the chemicals in some cultured cells and some tissues of experimental animals. All these biologic effects are thought to be mediated by an intracellular aryl hydrocarbon receptor (AHR). By fluorescence in situ hybridization and by DNA blot hybridization using human/mouse or human/Chinese hamster hybrid cell DNAs, Ema et al. (1994) assigned the AHR gene to 7p21. By use of PCR analysis of somatic cell hybrids and fluorescence in situ hybridization of metaphase cells, Le Beau et al. (1994) localized the AHR gene to 7p21-p15. Micka et al. (1997) localized the AHR gene to 7p15 using fluorescence in situ hybridization. Performing linkage analysis in a 3-generation family, they showed with good probability that the high CYP1A1 (OMIM Ref. No. 108330) inducibility phenotype segregates with the 7p15 region. Animal model experiments lend further support to the function of AHR. To determine whether the aryl hydrocarbon receptor plays a role in modulating carcinogenesis through the induction of xenobiotic-metabolizing enzymes, Shimizu et al. (2000) studied Ahr-deficient mice exposed to benzo(a)pyrene, a widely distributed environmental carcinogen.

It is appreciated that the abovementioned animal model for AHR is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ema, M.; Matsushita, N.; Sogawa, K.; Ariyama, T.; Inazawa, J.; Nemoto, T.; Ota, M.; Oshimura, M.; Fujii-Kuriyama, Y.: Human arylhydrocarbon receptor: functional expression and chromosomal assignment to 7p21. J. Biochem. 116: 845-851, 1994.; and
Shimizu, Y.; Nakatsuru, Y.; Ichinose, M.; Takahashi, Y.; Kume, H.; Mimura, J.; Fujii-Kuriyama, Y.; Ishikawa, T.: Benzo[a]pyrene carcinogenicity is lost in mice lacking the aryl hydrocar.

Further studies establishing the function and utilities of AHR are found in John Hopkins OMIM database record ID 600253, and in sited publications numbered 1883-1885, 175 and 1886-1887 listed in the bibliography section hereinbelow, which are also hereby incorporated by reference. Bassoon (presynaptic cytomatrix protein) (BSN, Accession NM_{—}003458) is another VGAM49 host target gene. BSN BINDING SITE1 and BSN BINDING SITE2 are HOST TARGET binding sites found in untranslated regions of mRNA encoded by BSN, corresponding to HOST TARGET binding sites such as BINDING SITE I, BINDING SITE II or BINDING SITE III. Table 2 illustrates the complementarity of the nucleotide sequences of BSN BINDING SITE1 and BSN BINDING SITE2, designated SEQ ID:1025 and SEQ ID: 1026 respectively, to the nucleotide sequence of VGAM49 RNA, herein designated VGAM RNA, also designated SEQ ID:384.

Another function of VGAM49 is therefore inhibition of Bassoon (presynaptic cytomatrix protein) (BSN, Accession NM_{—}003458), a gene which may be involved in cytomatrix organization at the site of neurotransmitter release. Accordingly, utilities of VGAM49 include diagnosis, prevention and treatment of diseases and clinical conditions associated with BSN. The function of BSN and its association with various diseases and clinical conditions, has been established by previous studies, as described hereinabove with reference to VGAM48. Cystic Fibrosis Transmembrane Conductance Regulator, ATP-binding Cassette (sub-family C, member 7) (CFTR, Accession NM_{—}000492) is another VGAM49 host target gene. CFTR BINDING SITE is HOST TARGET binding site found in the 3′ untranslated region of mRNA encoded by CFTR, corresponding to a HOST TARGET binding site such as BINDING SITE I, BINDING SITE II or BINDING SITE III. Table 2 illustrates the complementarity of the nucleotide sequences of CFTR BINDING SITE, designated SEQ ID:753, to the nucleotide sequence of VGAM49 RNA, herein designated VGAM RNA, also designated SEQ ID:384.

Another function of VGAM49 is therefore inhibition of Cystic Fibrosis Transmembrane Conductance Regulator, ATP-binding Cassette (sub-family C, member 7) (CFTR, Accession NM_{—}000492). Accordingly, utilities of VGAM49 include diagnosis, prevention and treatment of diseases and clinical conditions associated with CFTR. Chromodomain Helicase DNA Binding Protein 2 (CHD2, Accession NM_{—}001271) is another VGAM49 host target gene. CHD2 BINDING SITE is HOST TARGET binding site found in the 3′ untranslated region of mRNA encoded by CHD2, corresponding to a HOST TARGET binding site such as BINDING SITE I, BINDING SITE II or BINDING SITE III. Table 2 illustrates the complementarity of the nucleotide sequences of CHD2 BINDING SITE, designated SEQ ID:813, to the nucleotide sequence of VGAM49 RNA, herein designated VGAM RNA, also designated SEQ ID:384.

Another function of VGAM49 is therefore inhibition of Chromodomain Helicase DNA Binding Protein 2 (CHD2, Accession NM_{—}001271). Accordingly, utilities of VGAM49 include diagnosis, prevention and treatment of diseases and clinical conditions associated with CHD2. Cockayne Syndrome 1 (classical) (CKN1, Accession NM_{—}000082) is another VGAM49 host target gene. CKN1 BINDING SITE is HOST TARGET binding site found in the 3′ untranslated region of mRNA encoded by CKN1, corresponding to a HOST TARGET binding site such as BINDING SITE I, BINDING SITE II or BINDING SITE III. Table 2 illustrates the complementarity of the nucleotide sequences of CKN1 BINDING SITE, designated SEQ ID:703, to the nucleotide sequence of VGAM49 RNA, herein designated VGAM RNA, also designated SEQ ID:384.

Another function of VGAM49 is therefore inhibition of Cockayne Syndrome 1 (classical) (CKN1, Accession NM_{—}000082). Accordingly, utilities of VGAM49 include diagnosis, prevention and treatment of diseases and clinical conditions associated with CKN1. Cystathionase (cystathionine gamma-lyase) (CTH, Accession NM_{—}001902) is another VGAM49 host target gene. CTH BINDING SITE is HOST TARGET binding site found in the 5′ untranslated region of mRNA encoded by CTH, corresponding to a HOST TARGET binding site such as BINDING SITE I, BINDING SITE II or BINDING SITE III. Table 2 illustrates the complementarity of the nucleotide sequences of CTH BINDING SITE, designated SEQ ID:867, to the nucleotide sequence of VGAM49 RNA, herein designated VGAM RNA, also designated SEQ ID:384.

Another function of VGAM49 is therefore inhibition of Cystathionase (cystathionine gamma-lyase) (CTH, Accession NM_{—}001902), a gene which catalyzes removal of ammonia, cleaves cystathionine to yield free cys. Accordingly, utilities of VGAM49 include diagnosis, prevention and treatment of diseases and clinical conditions associated with CTH. The function of CTH has been established by previous studies. The rate of glutathione synthesis in the liver is limited by the availability of cysteine which is provided either in the diet or by the trans-sulfuration pathway in which cystathionine derived from methionine is transformed into cysteine by cystathionase (cystathionine gamma-lyase; EC 4.4.1.1). Lu et al. (1992) found and sequenced a 1,083-bp cDNA encoding the human liver cystathionine gamma-lyase. The human sequence presented a deletion of 132 bases (44 amino acids) compared to the cystathionase sequence reported for the rat, and a deletion of 135 bases (45 amino acids) compared to the sequence reported for yeast. After realignment for the missing nucleotides, the human cDNA sequence showed significant amino acid homology to the rat (85%) and yeast (50%) enzymes. Using the polymerase chain reaction, Lu et al. (1992) isolated a second clone which contained the missing 132 bases. Flanking nucleotides in the latter clone were identical to those in the cDNA clone containing the deletion. Alternative splicing as the basis of the 2 forms of cystathionase is plausible During a survey by paper chromatography of amino acids in the urine of patients in an institution for mental defectives, Harris et al. (1959) discovered a case with abnormal excretion of cystathionine. An inborn error involving the cleavage of cystathionine to give cysteine and homoserine was suggested. The subject was a severely retarded female, aged 64 years at the time of study. Another case was studied at The New York Hospital. Other clinical manifestations have been clubfoot, developmental defects about the ears, convulsions, and thrombocytopenia. Urinary lithiasis also occurs. Frimpter (1965) has shown that the defect involves cystathionase that does not properly bind its coenzyme, pyridoxal phosphate. In vitro studies suggested that high pyridoxine would be therapeutically beneficial. Mongeau et al. (1967) described the case of a 2-year-old boy with normal mentality, thrombocytopenia, and urinary calculi. The relation of the latter two features to the metabolic defect was problematical. Both parents (who were apparently unrelated) showed cystathioninuria after methionine loading test. With administration of pyridoxine, cystathioninuria was diminished in the proband. Schneiderman (1967) studied 2 mentally retarded brothers who excreted large amounts of cystathionine after methionine ingestion. The mother and another brother excreted lesser but abnormal amounts after methionine loading. The father was not tested. Perry et al. (1968) discovered cystathioninuria in a brother and sister when the brother's urine was by chance subjected to 2-dimensional paper chromatography for amino acids. Both children were normal. The parents excreted cystathionine only after methionine loading. The authors suggested that mental defect and other disorders reported in association with cystathioninuria may have been coincidental. Whelan and Scriver (1968) also found cystathioninuria as an apparently benign inborn error. The case of Tada et al. (1968) did not respond to vitamin

Full details of the abovementioned studies are described in the following publications, the disclosure of which are hereby incorporated by reference:
Lu, Y.; O'Dowd, B. F.; Orrego, H.; Israel, Y.: Cloning and nucleotide sequence of human liver cDNA encoding for cystathionine gamma-lyase. Biochem. Biophys. Res. Commun. 189: 749-758, 1992.; and
Whelan, D. T.; Scriver, C. R.: Cystathioninuria and renal iminoglycinuria in a pedigree: a perspective on counseling. New Eng. J. Med. 278: 924-927, 1968.

Further studies establishing the function and utilities of CTH are found in John Hopkins OMIM database record ID 219500, and in sited publications numbered 2106-2120 listed in the bibliography section hereinbelow, which are also hereby incorporated by reference. Epithelial V-like Antigen 1 (EVA1, Accession NM_{—}005797) is another VGAM49 host target gene. EVA1 BINDING SITE is HOST TARGET binding site found in the 3′ untranslated region of mRNA encoded by EVA1, corresponding to a HOST TARGET binding site such as BINDING SITE I, BINDING SITE II or BINDING SITE III. Table 2 illustrates the complementarity of the nucleotide sequences of EVA1 BINDING SITE, designated SEQ ID:1251, to the nucleotide sequence of VGAM49 RNA, herein designated VGAM RNA, also designated SEQ ID:384.

Another function of VGAM49 is therefore inhibition of Epithelial V-like Antigen 1 (EVA1, Accession NM_{—}005797). Accordingly, utilities of VGAM49 include diagnosis, prevention and treatment of diseases and clinical conditions associated with EVA1. F-box and Leucine-rich Repeat Protein 11 (FBXL11, Accession NM_{—}012308) is another VGAM49 host target gene. FBXL11 BINDING SITE is HOST TARGET binding site found in the 3′ untranslated region of mRNA encoded by FBXL11, corresponding to a HOST TARGET binding site such as BINDING SITE I, BINDING SITE II or BINDING SITE III. Table 2 illustrates the complementarity of the nucleotide sequences of FBXL11 BINDING SITE, designated SEQ ID: 1426, to the nucleotide sequence of VGAM49 RNA, herein designated VGAM RNA, also designated SEQ ID:384.

Another function of VGAM49 is therefore inhibition of F-box and Leucine-rich Repeat Protein 11 (FBXL11, Accession NM_{—}012308), a gene which are BTB/POZ domain-containing zinc finger proteins implicated in oncogenesis. Accordingly, utilities of VGAM49 include diagnosis, prevention and treatment of diseases and clinical conditions associated with FBXL11. The function of FBXL11 has been established by previous studies. The F box, named after cyclin F (CCNF; 600227), in which it was originally observed, is an approximately 40-amino acid motif that binds SKP1 (OMIM Ref. No. 601434). F-box proteins are components of modular E3 ubiquitin protein ligases called SCFs (SKP1, OMIM Ref. No. 603134), F-box proteins), which function in phosphorylation-dependent ubiquitination. Using a yeast 2-hybrid screen with SKP1 as bait, followed by searching sequence databases, Winston et al. (1999) and Cenciarelli et al. (1999) identified 33 mammalian and 26 human F-box proteins, respectively. These contained C termini with leucine-rich repeats (FBXLs, e.g., SKP2 (OMIM Ref. No. 601436)), WD40 domains (FBXWs, e.g., BTRCP (OMIM Ref. No. 603482)), or no recognizable motifs (FBXOs, e.g., CCNF). By searching sequence databases, Ilyin et al. (2000) identified a cDNA encoding FBXL11, which they referred to as FBL7. They predicted that FBXL11, which is identical to the 496-amino acid KIAA1004 protein reported by Nagase et al. (1999), contains at least 6 highly degenerated leucine-rich repeats. By RT-PCR analysis, Nagase et al. (1999) detected ubiquitous expression of FBXL11, with highest levels in brain, testis, and ovary, followed by lung; lowest expression was in pancreas. Within brain, expression was highest in cerebellum and subthalamic nuclei. The International Radiation Hybrid Mapping Consortium mapped the FBXL11 gene to chromosome 11 (sts-Z40471).

Full details of the abovementioned studies are described in the following publications, the disclosure of which are hereby incorporated by reference:
Ilyin, G. P.; Rialland, M.; Pigeon, C.; Guguen-Guillouzo, C.: cDNA cloning and expression analysis of new members of the mammalian F-box protein family. Genomics 67: 40-47, 2000.; and
Nagase, T.; Ishikawa, K.; Suyama, M.; Kikuno, R.; Hirosawa, M.; Miyajima, N.; Tanaka, A.; Kotani, H.; Nomura, N.; Ohara, O.: Prediction of the coding sequences of unidentified human gene.

Further studies establishing the function and utilities of FBXL11 are found in John Hopkins OMIM database record ID 605657, and in sited publications numbered 75, 188 and 1941 listed in the bibliography section hereinbelow, which are also hereby incorporated by reference. GATA Binding Protein 3 (GATA3, Accession NM_{—}002051) is another VGAM49 host target gene. GATA3 BINDING SITE is HOST TARGET binding site found in the 3′ untranslated region of mRNA encoded by GATA3, corresponding to a HOST TARGET binding site such as BINDING SITE I, BINDING SITE II or BINDING SITE III. Table 2 illustrates the complementarity of the nucleotide sequences of GATA3 BINDING SITE, designated SEQ ID:893, to the nucleotide sequence of VGAM49 RNA, herein designated VGAM RNA, also designated SEQ ID:384.

Another function of VGAM49 is therefore inhibition of GATA Binding Protein 3 (GATA3, Accession NM_{—}002051). Accordingly, utilities of VGAM49 include diagnosis, prevention and treatment of diseases and clinical conditions associated with GATA3. GDP Dissociation Inhibitor 2 (GDI2, Accession NM_{—}001494) is another VGAM49 host target gene. GDI2 BINDING SITE is HOST TARGET binding site found in the 5′ untranslated region of mRNA encoded by GDI2, corresponding to a HOST TARGET binding site such as BINDING SITE I, BINDING SITE II or BINDING SITE III. Table 2 illustrates the complementarity of the nucleotide sequences of GDI2 BINDING SITE, designated SEQ ID:832, to the nucleotide sequence of VGAM49 RNA, herein designated VGAM RNA, also designated SEQ ID:384.

Another function of VGAM49 is therefore inhibition of GDP Dissociation Inhibitor 2 (GDI2, Accession NM_{—}001494), a gene which regulates the gdp/gtp exchange reaction of most rab proteins by inhibiting the dissociation of gdp from them, and the subsequent binding of gtp to them. Accordingly, utilities of VGAM49 include diagnosis, prevention and treatment of diseases and clinical conditions associated with GDI2. The function of GDI2 has been established by previous studies. Various rab GDI-beta (RABGDIB) genes have been identified in a variety of species. It is a member of the GDP-dissociation inhibitor family, which includes GDI-alpha (RABGDIA; 300104). Shisheva et al. (1994) cloned mouse RABGDIB (which they referred to as ‘smg p25A GDI’) and reported the sequence. Sedlacek et al. (1995) found that the human RABGDIB sequence is 86.5% similar to RABGDIA, which they referred to as ‘XAP-4.’ Bachner et al. (1995) studied expression patterns of the 2 human genes. They showed that the 2.5-kb mRNA for RABGDIB is ubiquitously expressed, in contrast to RABGDIA, which is expressed primarily in neural and sensory tissues. By in situ hybridization, Sedlacek et al. (1998) demonstrated that the GDI2 gene maps to 10p15; a processed pseudogene mapped to 7p13-p11.

Full details of the abovementioned studies are described in the following publications, the disclosure of which are hereby incorporated by reference:
Sedlacek, Z.; Munstermann, E.; Mincheva, A.; Lichter, P.; Poustka, A.: The human rab GDI beta gene with long retroposon-rich introns maps to 10p15 and its pseudogene to 7p11-p13. Mammalian Genome 9: 78-80, 1998.; and
Shisheva, A.; Sudhof, T. C.; Czech, M. P.: Cloning, characterization, and expression of a novel GDP dissociation inhibitor isoform from skeletal muscle. Molec. Cell. Biol. 14: 3459-346.

Further studies establishing the function and utilities of GDI2 are found in John Hopkins OMIM database record ID 600767, and in sited publications numbered 1992, 2166-216 and 2030 listed in the bibliography section hereinbelow, which are also hereby incorporated by reference. Huntingtin (Huntington disease) (HD, Accession NM_{—}002111) is another VGAM49 host target gene. HD BINDING SITE is HOST TARGET binding site found in the 3′ untranslated region of mRNA encoded by HD, corresponding to a HOST TARGET binding site such as BINDING SITE I, BINDING SITE II or BINDING SITE III. Table 2 illustrates the complementarity of the nucleotide sequences of HD BINDING SITE, designated SEQ ID:899, to the nucleotide sequence of VGAM49 RNA, herein designated VGAM RNA, also designated SEQ ID:384.

Another function of VGAM49 is therefore inhibition of Huntingtin (Huntington disease) (HD, Accession NM_{—}002111). Accordingly, utilities of VGAM49 include diagnosis, prevention and treatment of diseases and clinical conditions associated with HD. 5-hydroxytryptamine (serotonin) Receptor 2C (HTR2C, Accession NM_{—}000868) is another VGAM49 host target gene. HTR2C BINDING SITE is HOST TARGET binding site found in the 3′ untranslated region of mRNA encoded by HTR2C, corresponding to a HOST TARGET binding site such as BINDING SITE I, BINDING SITE II or BINDING SITE III. Table 2 illustrates the complementarity of the nucleotide sequences of HTR2C BINDING SITE, designated SEQ ID:781, to the nucleotide sequence of VGAM49 RNA, herein designated VGAM RNA, also designated SEQ ID:384.

Another function of VGAM49 is therefore inhibition of 5-hydroxytryptamine (serotonin) Receptor 2C (HTR2C, Accession NM_{—}000868), a gene which activates phospholipase C and regulates intracellular calcium flux. Accordingly, utilities of VGAM49 include diagnosis, prevention and treatment of diseases and clinical conditions associated with HTR2C. The function of HTR2C has been established by previous studies. Hall (1947) found that DBA/2 mice exhibited genetic susceptibility to audiogenic seizures (AGSs) stimulated by a doorbell mounted in an iron tub. Thereafter, audiogenic seizures were among the intensively studied phenotypes in behavioral genetics. Brennan et al. (1997) found that null mutant mice lacking serotonin 5-HT-2C receptors are extremely susceptible to AGSs. The onset of susceptibility was between 2 and 3 months of age, with complete penetrance in adult animals. Heisler et al. (2002) hypothesized that 5-HT receptors are expressed in POMC (OMIM Ref. No. 176830) neurons and that action at these receptors mediates a component of the anorexic effect of d-FEN (D-fenfluramine). Heisler et al. (2002) found that up to 80% of alpha-MSH neurons express HTR2C mRNA and that the pattern of coexpression was greatest in the caudal arcuate nucleus of the hypothalamus. Heisler et al. (2002) demonstrated that direct activation of HTR2C by agonist in rats decreased their food intake and showed increased induction of FOS-like immunoreactivity in a pattern persistent with dFEN-induced FOS-like immunoreactivity expression in the arcuate nucleus and paraventricular nucleus of the hypothalamus. Heisler et al. (2002) demonstrated that dFEN directly activates POMC neurons, indicating that central 5-HT systems directly activate POMC neurons.

Full details of the abovementioned studies are described in the following publications, the disclosure of which are hereby incorporated by reference:
Heisler, L. K.; Cowley, M. A.; Tecott, L. H.; Fan, W.; Low, M. J.; Smart, J. L.; Rubinstein, M.; Tatro, J. B.; Marcus, J. N.; Holstege, H.; Lee, C. E.; Cone, R. D.; Elmquist, J. K.: Activation of central melanocortin pathways by fenfluramine. Science 297: 609-611, 2002.; and
Tecott, L. H.; Sun, L. M.; Akana, S. F.; Strack, A. M.; Lowenstein, D. H.; Dallman, M. F.; Julius, D.: Eating disorder and epilepsy in mice lacking 5-HT2C serotonin receptors. Nature 3.

Further studies establishing the function and utilities of HTR2C are found in John Hopkins OMIM database record ID 312861, and in sited publications numbered 2426-242 and 2659-2431 listed in the bibliography section hereinbelow, which are also hereby incorporated by reference. LGR6 (Accession XM_{—}097508) is another VGAM49 host target gene. LGR6 BINDING SITE is HOST TARGET binding site found in the 3′ untranslated region of mRNA encoded by LGR6, corresponding to a HOST TARGET binding site such as BINDING SITE I, BINDING SITE II or BINDING SITE III. Table 2 illustrates the complementarity of the nucleotide sequences of LGR6 BINDING SITE, designated SEQ ID:3302, to the nucleotide sequence of VGAM49 RNA, herein designated VGAM RNA, also designated SEQ ID:384.

Another function of VGAM49 is therefore inhibition of LGR6 (Accession XM_{—}097508). Accordingly, utilities of VGAM49 include diagnosis, prevention and treatment of diseases and clinical conditions associated with LGR6. Low Density Lipoprotein Receptor-related Protein 8, Apolipoprotein E Receptor (LRP8, Accession NM_{—}033300) is another VGAM49 host target gene. LRP8 BINDING SITE1 and LRP8 BINDING SITE2 are HOST TARGET binding sites found in untranslated regions of mRNA encoded by LRP8, corresponding to HOST TARGET binding sites such as BINDING SITE I, BINDING SITE II or BINDING SITE III. Table 2 illustrates the complementarity of the nucleotide sequences of LRP8 BINDING SITE1 and LRP8 BINDING SITE2, designated SEQ ID:2326 and SEQ ID:1139 respectively, to the nucleotide sequence of VGAM49 RNA, herein designated VGAM RNA, also designated SEQ ID:384.

Another function of VGAM49 is therefore inhibition of Low Density Lipoprotein Receptor-related Protein 8, Apolipoprotein E Receptor (LRP8, Accession NM_{—}033300), a gene which binds vldI and transports it into cells by endocytosis. Accordingly, utilities of VGAM49 include diagnosis, prevention and treatment of diseases and clinical conditions associated with LRP8. The function of LRP8 has been established by previous studies. Apolipoprotein E (APOE; 107741) is a 34-kD lipophilic protein that mediates high-affinity binding of APOE-containing lipoproteins to the low density lipoprotein receptor (see OMIM Ref. No. LDLR; 606945) and the very low density lipoprotein receptor (VLDLR; 192977). By screening a human placenta cDNA library with degenerate oligonucleotides based on a highly conserved region between LDLR and VLDLR, Kim et al. (1996) identified a cDNA encoding APOE receptor-2 (OMIM Ref. No. APOER2). The predicted 963-amino acid protein contains a putative 41-amino acid signal sequence and 5 functional domains that resemble those of LDLR and VLDLR. APOER2 appears specific for APOE-containing ligands: LDLR-deficient mammalian cells expressing APOER bound APOE-rich beta-VLDL with high affinity, but bound LDL and VLDL with much lower affinities. Northern blot analysis revealed that APOER2 is expressed as 4.5- and 8.5-kb mRNAs in brain and placenta Kim et al. (1997) reported that the APOER2 gene contains 19 exons and spans approximately 60 kb. Alternative splicing generates multiple transcripts encoding receptors with different numbers of cysteine-rich repeats in the ligand-binding domain

Full details of the abovementioned studies are described in the following publications, the disclosure of which are hereby incorporated by reference:
Kim, D.-H.; Iijima, H.; Goto, K.; Sakai, J.; Ishii, H.; Kim, H.-J.; Suzuki, H.; Kondo, H.; Saeki, S.; Yamamoto, T.: Human apolipoprotein E receptor 2: a novel lipoprotein receptor of the low density lipoprotein receptor family predominantly expressed in brain. J. Biol. Chem. 271: 8373-8380, 1996.; and
Kim, D.-H.; Magoori, K.; Inoue, T. R.; Mao, C. C.; Kim, H.-J.; Suzuki, H.; Fujita, T.; Endo, Y.; Saeki, S.; Yamamoto, T. T.: Exon/intron organization, chromosome localization, alternative.

Further studies establishing the function and utilities of LRP8 are found in John Hopkins OMIM database record ID 602600, and in sited publications numbered 2266-188 and 2267 listed in the bibliography section hereinbelow, which are also hereby incorporated by reference. Macrophage Scavenger Receptor 1 (MSR1, Accession NM_{—}002445) is another VGAM49 host target gene. MSR1 BINDING SITE is HOST TARGET binding site found in the 3′ untranslated region of mRNA encoded by MSR1, corresponding to a HOST TARGET binding site such as BINDING SITE I, BINDING SITE II or BINDING SITE III. Table 2 illustrates the complementarity of the nucleotide sequences of MSR1 BINDING SITE, designated SEQ ID:929, to the nucleotide sequence of VGAM49 RNA, herein designated VGAM RNA, also designated SEQ ID:384.

Another function of VGAM49 is therefore inhibition of Macrophage Scavenger Receptor 1 (MSR1, Accession NM_{—}002445), a gene which plays a role in endocytosis of macromolecules. Accordingly, utilities of VGAM49 include diagnosis, prevention and treatment of diseases and clinical conditions associated with MSR1. The function of MSR1 has been established by previous studies. Deletions on 8p23-p22 in prostate cancer cells (Latil and Lidereau, 1998) and linkage studies in families affected with hereditary prostate cancer (Xu et al., 2001) have implicated this region in the development of prostate cancer. The MSR1 gene is located at 8p22 and functions in several processes proposed to be relevant to prostate carcinogenesis. In studies of families affected with hereditary prostate cancer, Xu et al. (2002) identified 6 missense mutations and 1 nonsense mutation in the MSR1 gene. A family-based linkage and association test indicated that these mutations cosegregate with prostate cancer (P=0.0007). In addition, among men of European descent, MSR1 mutations were detected in 4.4% of individuals affected with nonhereditary prostate cancer as compared with 0.8% of unaffected men (P=0.009). Among African American men, these values were 12.5% and 1.8%, respectively (P=0.01). These results showed that MSR1 may be important in susceptibility to prostate cancer in men of both African American and European descent.

Full details of the abovementioned studies are described in the following publications, the disclosure of which are hereby incorporated by reference:
Emi, M.; Asaoka, H.; Matsumoto, A.; Itakura, H.; Kurihara, Y.; Wada, Y.; Kanamori, H.; Yazaki, Y.; Takahashi, E.; Lepert, M.; Lalouel, J.-M.; Kodama, T.; Mukai, T.: Structure, organization, and chromosomal mapping of the human macrophage scavenger receptor gene. J. Biol. Chem. 268: 2120-2125, 1993.; and
Latil, A.; Lidereau, R.: Genetic aspects of prostate cancer. Virchows Arch. 432: 389-406, 1998.

Further studies establishing the function and utilities of MSR1 are found in John Hopkins OMIM database record ID 153622, and in sited publications numbered 2689-2693 listed in the bibliography section hereinbelow, which are also hereby incorporated by reference. Proto-cadherin 11 X-linked (PCDH11X, Accession NM_{—}032968) is another VGAM49 host target gene. PCDH11X BINDING SITE1 and PCDH11X BINDING SITE2 are HOST TARGET binding sites found in untranslated regions of mRNA encoded by PCDH11X, corresponding to HOST TARGET binding sites such as BINDING SITE I, BINDING SITE II or BINDING SITE III. Table 2 illustrates the complementarity of the nucleotide sequences of PCDH11X BINDING SITE1 and PCDH11X BINDING SITE2, designated SEQ ID:2299 and SEQ ID:2301 respectively, to the nucleotide sequence of VGAM49 RNA, herein designated VGAM RNA, also designated SEQ ID:384.

Another function of VGAM49 is therefore inhibition of Proto-cadherin 11 X-linked (PCDH11X, Accession NM_{—}032968), a gene which is thought to play a fundamental role in cell-cell recognition essential for the segmental development and function of the central nervous system. Accordingly, utilities of VGAM49 include diagnosis, prevention and treatment of diseases and clinical conditions associated with PCDH11X. The function of PCDH11X has been established by previous studies. The protocadherins are a sub family of calcium-dependent cell adhesion and recognition proteins of the cadherin super family. They are particularly prevalent in the central nervous system. By database searching with a human PCDH7 (OMIM Ref. No. 602988) cDNA probe, Yoshida and Sugano (1999) identified a homologous mRNA from infant brain. They found the same sequence on a genomic clone of chromosome X, within the X-Y homology region at Xp21.3. By RT-PCR using fetal brain, they identified a cDNA, PCDH11, which they called PCDHX, encoding a deduced 1,021-amino acid protein. Sequence analysis predicted a 25-amino acid signal peptide, an extracellular portion with 7 cadherin repeats, a 24-amino acid transmembrane stretch, and a 10-amino acid cytoplasmic domain with homology to PCDH1 (OMIM Ref. No. 603626) and PCDH7. Yoshida and Sugano (1999) also identified a splice variant, termed PCDHXb, by EST database searches. Northern blot analysis revealed expression of an approximately 6.0-kb transcript in human and mouse fetal brain. Using STS analysis, Yoshida and Sugano (1999) mapped the PCDH11 gene to the X-Y homology region at chromosome Xq21.3 that is homologous to Yp11.1. Blanco et al. (2000) mapped the PCDH11 gene to Xq21.3, within the XY homologous region, using detailed YAC and PAC contigs and fine STS marker order.

Full details of the abovementioned studies are described in the following publications, the disclosure of which are hereby incorporated by reference:
Yoshida, K.; Sugano, S.: Identification of a novel proto-cadherin gene (PCDH11) on the human XY homology region in Xq21.3. Genomics 62: 540-543, 1999.; and
Yoshida, K.; Sugano, S.: Identification of a novel proto-cadherin gene (PCDH11) on the human XY homology region in Xq21.3. Genomics 62: 540-543, 1999.

Further studies establishing the function and utilities of PCDH11X are found in John Hopkins OMIM database record ID 300246, and in sited publications numbered 2059-2064 listed in the bibliography section hereinbelow, which are also hereby incorporated by reference. Protein-L-isoaspartate (D-aspartate) O-methyltransferase (PCMT1, Accession NM_{—}005389) is another VGAM49 host target gene. PCMT1 BINDING SITE is HOST TARGET binding site found in the 3′ untranslated region of mRNA encoded by PCMT1, corresponding to a HOST TARGET binding site such as BINDING SITE I, BINDING SITE II or BINDING SITE III. Table 2 illustrates the complementarity of the nucleotide sequences of PCMT1 BINDING SITE, designated SEQ ID:1205, to the nucleotide sequence of VGAM49 RNA, herein designated VGAM RNA, also designated SEQ ID:384.

Another function of VGAM49 is therefore inhibition of Protein-L-isoaspartate (D-aspartate) O-methyltransferase (PCMT1, Accession NM_{—}005389), a gene which catalyzes the methyl esterification of 1-isoaspartyl and d-aspartyl residues in peptides and proteins. Accordingly, utilities of VGAM49 include diagnosis, prevention and treatment of diseases and clinical conditions associated with PCMT1. The function of PCMT1 has been established by previous studies. Three classes of protein carboxyl methyltransferases, distinguished by their methyl-acceptor substrate specificity, have been found in prokaryotic and eukaryotic cells. The type II enzyme catalyzes the transfer of a methyl group from S-adenosyl-L-methionine to the free carboxyl groups of D-aspartyl and L-isoaspartyl residues. These methyl-accepting residues result from the spontaneous deamidation, isomerization, and racemization of normal L-aspartyl and L-asparaginyl residues and represent sites of covalent damage to aging proteins PCMT1 (EC 2.1.1.77) is a protein repair enzyme that initiates the conversion of abnormal D-aspartyl and L-isoaspartyl residues to the normal L-aspartyl form. L-isoaspartyl-D-aspartyl methyltransferase is a cytosolic monomer of about 25 kD. Ingrosso et al. (1991) and MacLaren et al. (1992) showed that 2 major isozymes of this transferase result by alternative splicing of a single gene product. Animal model experiments lend further support to the function of PCMT1. Kim et al. (1999) developed Pcmt1 null mice. These mice manifested 2 phenotypes, a fatal seizure disorder and retarded growth. Continuous electroencephalogram monitoring of Pcmt1 null mice revealed that abnormal cortical activity occurred for about half of each 24-hour period, even in mice that had no visible evidence of convulsions. Antiepileptic therapy mitigated but did not eliminate the seizure disorder. It did, however, normalize the growth of Pcmt1 null mice, suggesting that the growth retardation was due to seizures rather than a global disturbance in growth at the cellular level. Consistent with this, the growth rate of Pcmt1 null fibroblasts was indistinguishable from that of wildtype fibroblasts.

It is appreciated that the abovementioned animal model for PCMT1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
MacLaren, D. C.; Kagan, R. M.; Clarke, S.: Alternative splicing of the human isoaspartyl protein carboxyl methyltransferase leads to the generation of a C-terminal -RDEL sequence in isozyme II. Biochem. Biophys. Res. Commun. 185: 277-283, 1992.; and
Kim et al. (1999) developed Pcmt1 null mice. These mice manifested 2 phenotypes, a fatal seizure disorder and retarded growth. Continuous electroencephalogram monitoring of Pcmt1 null mice.

Further studies establishing the function and utilities of PCMT1 are found in John Hopkins OMIM database record ID 176851, and in sited publications numbered 1237-1244 listed in the bibliography section hereinbelow, which are also hereby incorporated by reference. Proliferating Cell Nuclear Antigen (PCNA, Accession NM_{—}002592) is another VGAM49 host target gene. PCNA BINDING SITE is HOST TARGET binding site found in the 3′ untranslated region of mRNA encoded by PCNA, corresponding to a HOST TARGET binding site such as BINDING SITE I, BINDING SITE II or BINDING SITE III. Table 2 illustrates the complementarity of the nucleotide sequences of PCNA BINDING SITE, designated SEQ ID:939, to the nucleotide sequence of VGAM49 RNA, herein designated VGAM RNA, also designated SEQ ID:384.

Another function of VGAM49 is therefore inhibition of Proliferating Cell Nuclear Antigen (PCNA, Accession NM_{—}002592), a gene which is involved in the control of eukaryotic dna replication by increasing the polymerase's processibility during elongation of the leading strand. Accordingly, utilities of VGAM49 include diagnosis, prevention and treatment of diseases and clinical conditions associated with PCNA. The function of PCNA has been established by previous studies. Proliferating cell nuclear antigen was originally identified by immunofluorescence as a nuclear protein whose appearance correlated with the proliferative state of the cell. A cell cycle-dependent protein described by Bravo (1986) and called cyclin was shown to be identical to PCNA. PCNA is required for replication of SV40 DNA in vitro and has been identified as the auxiliary protein (cofactor) for DNA polymerase delta (OMIM Ref. No. 174761). Unlike DNA polymerases alpha (OMIM Ref. No. 312040), beta (OMIM Ref. No. 174760), and gamma (OMIM Ref. No. 174763), DNA polymerase delta has exonuclease activity. Since the exonuclease activity is in the 3-prime-to-5-prime direction, DNA polymerase delta has a proofreading activity and is expected to play a significant role in the maintenance of the fidelity of mammalian DNA replication. The protein has been highly conserved during evolution; the deduced amino acid sequences of rat and human differ by only 4 out of 261 amino acids. The human anti-PCNA autoantibodies react not only with the nuclei of proliferating cells of all experimental animals so far examined but also with the nuclei of plant cells. Suzuka et al. (1989) demonstrated the presence of the PCNA/cyclin-related genes in higher plants. It had been speculated that genetic engineering could improve the long-term function of vascular grafts that are prone to atherosclerosis and occlusion. Mann et al. (1995) showed that an ‘intraoperative gene therapy approach’ using antisense oligodeoxynucleotides to block medial smooth muscle cell proliferation can prevent the accelerated atherosclerosis that is responsible for autologous vein graft failure. Selective prevention of the expression of genes for 2 cell cycle regulatory proteins, proliferating cell nuclear antigen and cell division cycle kinase 2 (CDK2; 116953), was achieved in the smooth muscle cells of rabbit jugular veins grafted into the carotid arteries. This alteration of gene expression successfully redirected vein graft biology away from neointimal hyperplasia and toward medial hypertrophy, yielding conduits that more closely resembled normal arteries. Furthermore, the genetically engineered grafts proved resistant to diet-induced atherosclerosis.

Full details of the abovementioned studies are described in the following publications, the disclosure of which are hereby incorporated by reference:
Bravo, R.: Synthesis of the nuclear protein cyclin (PCNA) and its relationship with DNA replication. Exp. Cell Res. 163: 287-293, 1986.; and
Mann, M. J.; Gibbons, G. H.; Kernoff, R. S.; Diet, F. P.; Tsao, P. S.; Cooke, J. P.; Kaneda, Y.; Dzau, V. J.: Genetic engineering of vein grafts resistant to atherosclerosis. Proc. Nat.

Further studies establishing the function and utilities of PCNA are found in John Hopkins OMIM database record ID 176740, and in sited publications numbered 374-383 listed in the bibliography section hereinbelow, which are also hereby incorporated by reference. Proline Arginine-rich End Leucine-rich Repeat Protein (PRELP, Accession NM_{—}002725) is another VGAM49 host target gene. PRELP BINDING SITE is HOST TARGET binding site found in the 3′ untranslated region of mRNA encoded by PRELP, corresponding to a HOST TARGET binding site such as BINDING SITE I, BINDING SITE II or BINDING SITE III. Table 2 illustrates the complementarity of the nucleotide sequences of PRELP BINDING SITE, designated SEQ ID:950, to the nucleotide sequence of VGAM49 RNA, herein designated VGAM RNA, also designated SEQ ID:384.

Another function of VGAM49 is therefore inhibition of Proline Arginine-rich End Leucine-rich Repeat Protein (PRELP, Accession NM_{—}002725), a gene which is a connective tissue glycoprotein of the leucine-rich-repeat (LRR) family. Accordingly, utilities of VGAM49 include diagnosis, prevention and treatment of diseases and clinical conditions associated with PRELP. The function of PRELP has been established by previous studies. PRELP (proline arginine-rich end leucine-rich repeat protein) is a connective tissue glycoprotein of the leucine-rich-repeat (LRR) family. The LRR motif is believed to participate in protein-protein interactions; Bengtsson et al. (1995) stated that this motif is found in several matrix-binding proteins. Bengtsson et al. (1995) isolated the 58-kD PRELP protein from bovine articular cartilage and cloned the human PRELP cDNA from an articular chondrocyte cDNA library. Sequencing revealed that the gene encodes a 382-amino acid polypeptide with a calculated molecular mass of 42 kD. The protein sequence contains a putative secretory signal sequence, 10 leucine-rich repeats, several glycosylation sites, and a proline- and arginine-rich N-terminus. They reported that the protein sequence was 36% similar to fibromodulin (OMIM Ref. No. 600245) and 33% similar to bovine lumican (see OMIM Ref. No. 600616). Grover et al. (1996) characterized the expression pattern and genomic structure of human PRELP. They determined that PRELP is encoded by messages of 1.7, 4.6, and 6.7 kb, in descending order of expression level. The messages are abundant in juvenile and adult cartilage, but not present in neonatal cartilage nor in cultured fibroblasts. Among other adult tissues, PRELP is expressed in lung. Grover et al. (1996) reported that the PRELP gene contains 2 introns. They mapped the gene to human chromosome 1q32; interestingly, the fibromodulin gene also resides at this location.

Full details of the abovementioned studies are described in the following publications, the disclosure of which are hereby incorporated by reference:
Bengtsson, E.; Neame, P. J.; Heinegard, D.; Sommarin, Y.: The primary structure of a basic leucine-rich repeat protein, PRELP, found in connective tissues. J. Biol. Chem. 270: 25639-25644, 1995.; and
Grover, J.; Chen, X.-N.; Korenberg, J. R.; Recklies, A. D.; Roughley, P. J.: The gene organization, chromosome location, and expression of a 55-kDa matrix protein (PRELP) of human arti.

Further studies establishing the function and utilities of PRELP are found in John Hopkins OMIM database record ID 601914, and in sited publications numbered 2057-2058 listed in the bibliography section hereinbelow, which are also hereby incorporated by reference. Parathymosin (PTMS, Accession NM_{—}002824) is another VGAM49 host target gene. PTMS BINDING SITE is HOST TARGET binding site found in the 3′ untranslated region of mRNA encoded by PTMS, corresponding to a HOST TARGET binding site such as BINDING SITE I, BINDING SITE II or BINDING SITE III. Table 2 illustrates the complementarity of the nucleotide sequences of PTMS BINDING SITE, designated SEQ ID:958, to the nucleotide sequence of VGAM49 RNA, herein designated VGAM RNA, also designated SEQ ID:384.

Another function of VGAM49 is therefore inhibition of Parathymosin (PTMS, Accession NM_{—}002824), a gene which is involved in the regulation of cellular immunity. Accordingly, utilities of VGAM49 include diagnosis, prevention and treatment of diseases and clinical conditions associated with PTMS. The function of PTMS and its association with various diseases and clinical conditions, has been established by previous studies, as described hereinabove with reference to VGAM48. Solute Carrier Family 4, Sodium Bicarbonate Cotransporter, Member 4 (SLC4A4, Accession NM_{—}003759) is another VGAM49 host target gene. SLC4A4 BINDING SITE is HOST TARGET binding site found in the 3′ untranslated region of mRNA encoded by SLC4A4, corresponding to a HOST TARGET binding site such as BINDING SITE I, BINDING SITE II or BINDING SITE III. Table 2 illustrates the complementarity of the nucleotide sequences of SLC4A4 BINDING SITE, designated SEQ ID: 1054, to the nucleotide sequence of VGAM49 RNA, herein designated VGAM RNA, also designated SEQ ID:384.

Another function of VGAM49 is therefore inhibition of Solute Carrier Family 4, Sodium Bicarbonate Cotransporter, Member 4 (SLC4A4, Accession NM_{—}003759), a gene which is a sodium bicarbonate cotransporter. Accordingly, utilities of VGAM49 include diagnosis, prevention and treatment of diseases and clinical conditions associated with SLC4A4. The function of SLC4A4 has been established by previous studies. By screening a human heart cDNA library with rat kidney Nbc cDNAs, followed by a PCR approach, Choi et al. (1999) isolated a full-length cDNA encoding a heart NBC, which they called hhNBC. They reported that the coding sequence of hhNBC is identical to that of pNBC (Abuladze et al., 1998). However, the 5-prime untranslated regions of hhNBC and pNBC differ. Northern blot analysis using the 5-prime region of the hhNBC coding sequence as probe detected an approximately 9-kb transcript that was strongly expressed in pancreas and weakly expressed in heart and brain. Choi et al. (1999) found that both hhNBC and kNBC (Burnham et al., 1997), when expressed in Xenopus, are electrogenic. Soleimani and Burnham (2000) stated that kNBC (Burnham et al., 1997) and pNBC (Abuladze et al., 1998) are encoded by splice variants of the same gene, SLC4A4, which they called NBC1. Mutations in the SLC4A4 gene (e.g., 603345.0001, 603345.0002) cause proximal renal tubular acidosis with bilateral glaucoma, cataracts, and band keratopathy (OMIM Ref. No. 604278). Such mutations may increase the bicarbonate concentration in the corneal stroma, which would facilitate calcium deposition leading to band keratopathy. Igarashi et al. (1999) suggested that the kidney and pancreatic NBCs are derived from a common gene by alternative splicing and that mutations at the common region would inactivate both isoforms. Studies by Usui et al. (1999) confirmed that both kidney and pancreatic NBC are involved in the transport of sodium and bicarbonate out of the corneal stroma and into the aqueous humor.

Full details of the abovementioned studies are described in the following publications, the disclosure of which are hereby incorporated by reference:
Soleimani, M.; Burnham, C. E.: Physiologic and molecular aspects of the Na(+):HCO(3−) cotransporter in health and disease processes. Kidney Int. 57: 371-384, 2000.; and
Choi, I.; Romero, M. F.; Khandoudi, N.; Bril, A.; Boron, W. F.: Cloning and characterization of a human electrogenic Na(+)—HCO(3−) cotransporter isoform (hhNBC). Am. J. Physiol. 276: C57.

Further studies establishing the function and utilities of SLC4A4 are found in John Hopkins OMIM database record ID 603345, and in sited publications numbered 1815-1821 listed in the bibliography section hereinbelow, which are also hereby incorporated by reference. Trichorhinophalangeal Syndrome I (TRPS1, Accession NM_{—}014112) is another VGAM49 host target gene. TRPS1 BINDING SITE is HOST TARGET binding site found in the 5′ untranslated region of mRNA encoded by TRPS1, corresponding to a HOST TARGET binding site such as BINDING SITE I, BINDING SITE II or BINDING SITE III. Table 2 illustrates the complementarity of the nucleotide sequences of TRPS1 BINDING SITE, designated SEQ ID: 1471, to the nucleotide sequence of VGAM49 RNA, herein designated VGAM RNA, also designated SEQ ID:384.

Another function of VGAM49 is therefore inhibition of Trichorhinophalangeal Syndrome I (TRPS1, Accession NM_{—}014112), a gene which may function as a transcriptional activator protein. Accordingly, utilities of VGAM49 include diagnosis, prevention and treatment of diseases and clinical conditions associated with TRPS1. The function of TRPS1 has been established by previous studies. Trichorhinophalangeal syndrome type I (OMIM Ref. No. 190350) is a malformation syndrome characterized by distinctive craniofacial and skeletal abnormalities and is inherited as an autosomal dominant. TRPS I patients have sparse scalp hair, bulbous tip of the nose, long flat philtrum, thin upper vermilion border, and protruding ears. Skeletal abnormalities include cone-shaped epiphyses at the phalanges, hip malformations, and short stature. Ludecke et al. (1995) and Hou et al. (1995) assigned the TRPS1 gene to 8q24. It maps centromeric to the gene that is mutant in multiple exostoses type I (EXT1; 133700); EXT1 is deleted in all patients with TRPS type II, or Langer-Giedion syndrome (OMIM Ref. No. 150230), which combines features of TRPS I and multiple exostoses. Momeni et al. (2000) positionally cloned a gene that spanned the chromosomal breakpoint in 2 patients with TRPS I and was deleted in 5 patients with TRPS I associated with an interstitial deletion. Northern blot analyses revealed transcripts of 7 and 10.5 kb. The gene, designated TRPS1, has 7 exons and encodes a polypeptide of 1,281 amino acids. The predicted protein sequence has 2 potential nuclear localization signals and an unusual combination of different zinc finger motifs, including IKAROS-like (see OMIM Ref. No. 603023) and GATA-binding (see OMIM Ref. No. 600576) sequences. Momeni et al. (2000) identified 6 different nonsense mutations in 10 unrelated patients. The findings suggested that haploinsufficiency for this putative transcription factor causes TRPS I. To investigate whether trichorhinophalangeal syndrome type III (OMIM Ref. No. 190351) is caused by TRPS1 mutations and to establish a genotype-phenotype correlation in TRPS, Ludecke et al. (2001) performed extensive mutation analysis and evaluated height and degree of brachydactyly in patients with TRPS I or TRPS III. They found 35 different mutations in 44 of 51 unrelated patients. The detection rate (86%) indicated that TRPS1 is the major locus for TRPS I and TRPS III. They found no mutation in the parents of sporadic patients or in apparently healthy relatives of familial patients, indicating complete penetrance of TRPS1 mutations. Evaluation of skeletal abnormalities of patients with TRPS1 mutations revealed a wide clinical spectrum. The phenotype was variable in unrelated, age- and sex-matched patients with identical mutations, as well as in families. Four of the 5 missense mutations altered the GATA DNA-binding zinc finger, and 6 of the 7 unrelated patients with these mutations could be classified as having TRPS III, because they had severe bradycardia, due to short metacarpals, and severe short stature. The data indicated that TRPS III is at the severe end of the TRPS spectrum and that it is most often caused by a specific class of mutations in exon 6 the TRPS1 gene. In the study of Ludecke et al. (2001), 5 mutations were recurrent, and 4 of these were identified in patients of different ethnicities: 1 in patients of Norwegian, Turkish, and Belgian extraction, and another in patients of Belgian, Turkish, and Japanese extraction, for example.

Full details of the abovementioned studies are described in the following publications, the disclosure of which are hereby incorporated by reference:
Momeni, P.; Glockner, G.; Schmidt, O.; von Holtum, D.; Albrecht, B.; Gillessen-Kaesbach, G.; Hennekam, R.; Meinecke, P.; Zabel, B.; Rosenthal, A.; Horsthemke, B.; Ludecke, H.-J.: Mutations in a new gene, encoding a zinc-finger protein, cause tricho-rhino-phalangeal syndrome type I. Nature Genet. 24: 71-74, 2000.; and
Ludecke, H.-J.; Schaper, J.; Meinecke, P.; Momeni, P.; Gross, S.; von Holtum, D.; Hirche, H.; Abramowicz, M. J.; Albrecht, B.; Apacik, C.; Christen, H.-J.; Claussen, U.; and 28 others: G.

Further studies establishing the function and utilities of TRPS1 are found in John Hopkins OMIM database record ID 604386, and in sited publications numbered 1620-1621, 855, 1814, 305 and 3044 listed in the bibliography section hereinbelow, which are also hereby incorporated by reference. ARS2 (Accession NM_{—}015908) is another VGAM49 host target gene. ARS2 BINDING SITE is HOST TARGET binding site found in the 5′ untranslated region of mRNA encoded by ARS2, corresponding to a HOST TARGET binding site such as BINDING SITE I, BINDING SITE II or BINDING SITE III. Table 2 illustrates the complementarity of the nucleotide sequences of ARS2 BINDING SITE, designated SEQ ID: 1650, to the nucleotide sequence of VGAM49 RNA, herein designated VGAM RNA, also designated SEQ ID:384.

Another function of VGAM49 is therefore inhibition of ARS2 (Accession NM_{—}015908). Accordingly, utilities of VGAM49 include diagnosis, prevention and treatment of diseases and clinical conditions associated with ARS2. Bromodomain Containing 4 (BRD4, Accession NM_{—}058243) is another VGAM49 host target gene. BRD4 BINDING SITE is HOST TARGET binding site found in the 3′ untranslated region of mRNA encoded by BRD4, corresponding to a HOST TARGET binding site such as BINDING SITE I, BINDING SITE II or BINDING SITE III. Table 2 illustrates the complementarity of the nucleotide sequences of BRD4 BINDING SITE, designated SEQ ID:2368, to the nucleotide sequence of VGAM49 RNA, herein designated VGAM RNA, also designated SEQ ID:384.

Another function of VGAM49 is therefore inhibition of Bromodomain Containing 4 (BRD4, Accession NM_{—}058243). Accordingly, utilities of VGAM49 include diagnosis, prevention and treatment of diseases and clinical conditions associated with BRD4. CAPN13 (Accession NM_{—}144575) is another VGAM49 host target gene. CAPN13 BINDING SITE is HOST TARGET binding site found in the 3′ untranslated region of mRNA encoded by CAPN13, corresponding to a HOST TARGET binding site such as BINDING SITE I, BINDING SITE II or BINDING SITE III. Table 2 illustrates the complementarity of the nucleotide sequences of CAPN13 BINDING SITE, designated SEQ ID:2485, to the nucleotide sequence of VGAM49 RNA, herein designated VGAM RNA, also designated SEQ ID:384.

Another function of VGAM49 is therefore inhibition of CAPN13 (Accession NM_{—}144575). Accordingly, utilities of VGAM49 include diagnosis, prevention and treatment of diseases and clinical conditions associated with CAPN13. Cat Eye Syndrome Chromosome Region, Candidate 6 (CECR6, Accession NM_{—}031890) is another VGAM49 host target gene. CECR6 BINDING SITE is HOST TARGET binding site found in the 3′ untranslated region of mRNA encoded by CECR6, corresponding to a HOST TARGET binding site such as BINDING SITE I, BINDING SITE II or BINDING SITE III. Table 2 illustrates the complementarity of the nucleotide sequences of CECR6 BINDING SITE, designated SEQ ID:2218, to the nucleotide sequence of VGAM49 RNA, herein designated VGAM RNA, also designated SEQ ID:384.

Another function of VGAM49 is therefore inhibition of Cat Eye Syndrome Chromosome Region, Candidate 6 (CECR6, Accession NM_{—}031890). Accordingly, utilities of VGAM49 include diagnosis, prevention and treatment of diseases and clinical conditions associated with CECR6. DKFZP43410714 (Accession XM_{—}098247) is another VGAM49 host target gene. DKFZP43410714 BINDING SITE is HOST TARGET binding site found in the 5′ untranslated region of mRNA encoded by DKFZP43410714, corresponding to a HOST TARGET binding site such as BINDING SITE I, BINDING SITE II or BINDING SITE III. Table 2 illustrates the complementarity of the nucleotide sequences of DKFZP43410714 BINDING SITE, designated SEQ ID:3362, to the nucleotide sequence of VGAM49 RNA, herein designated VGAM RNA, also designated SEQ ID:384.

Another function of VGAM49 is therefore inhibition of DKFZP43410714 (Accession XM_{—}098247). Accordingly, utilities of VGAM49 include diagnosis, prevention and treatment of diseases and clinical conditions associated with DKFZP43410714. DKFZP564G092 (Accession NM_{—}015601) is another VGAM49 host target gene. DKFZP564G092 BINDING SITE is HOST TARGET binding site found in the 5′ untranslated region of mRNA encoded by DKFZP564G092, corresponding to a HOST TARGET binding site such as BINDING SITE I, BINDING SITE II or BINDING SITE III. Table 2 illustrates the complementarity of the nucleotide sequences of DKFZP564G092 BINDING SITE, designated SEQ ID: 1639, to the nucleotide sequence of VGAM49 RNA, herein designated VGAM RNA, also designated SEQ ID:384.

Another function of VGAM49 is therefore inhibition of DKFZP564G092 (Accession NM_{—}015601). Accordingly, utilities of VGAM49 include diagnosis, prevention and treatment of diseases and clinical conditions associated with DKFZP564G092. DKFZp564K142 (Accession NM_{—}032121) is another VGAM49 host target gene. DKFZp564K142 BINDING SITE is HOST TARGET binding site found in the 3′ untranslated region of mRNA encoded by DKFZp564K142, corresponding to a HOST TARGET binding site such as BINDING SITE I, BINDING SITE II or BINDING SITE III. Table 2 illustrates the complementarity of the nucleotide sequences of DKFZp564K142 BINDING SITE, designated SEQ ID:2231, to the nucleotide sequence of VGAM49 RNA, herein designated VGAM RNA, also designated SEQ ID:384.

Another function of VGAM49 is therefore inhibition of DKFZp564K142 (Accession NM_{—}032121). Accordingly, utilities of VGAM49 include diagnosis, prevention and treatment of diseases and clinical conditions associated with DKFZp564K142. DKFZp566D234 (Accession XM_{—}030162) is another VGAM49 host target gene. DKFZp566D234 BINDING SITE is HOST TARGET binding site found in the 3′ untranslated region of mRNA encoded by DKFZp566D234, corresponding to a HOST TARGET binding site such as BINDING SITE I, BINDING SITE II or BINDING SITE III. Table 2 illustrates the complementarity of the nucleotide sequences of DKFZp566D234 BINDING SITE, designated SEQ ID:2612, to the nucleotide sequence of VGAM49 RNA, herein designated VGAM RNA, also designated SEQ ID:384.

Another function of VGAM49 is therefore inhibition of DKFZp566D234 (Accession XM_{—}030162). Accordingly, utilities of VGAM49 include diagnosis, prevention and treatment of diseases and clinical conditions associated with DKFZp566D234. DKFZP56611024 (Accession XM_{—}046506) is another VGAM49 host target gene. DKFZP56611024 BINDING SITE is HOST TARGET binding site found in the 3′ untranslated region of mRNA encoded by DKFZP56611024, corresponding to a HOST TARGET binding site such as BINDING SITE I, BINDING SITE II or BINDING SITE III. Table 2 illustrates the complementarity of the nucleotide sequences of DKFZP56611024 BINDING SITE, designated SEQ ID:2878, to the nucleotide sequence of VGAM49 RNA, herein designated VGAM RNA, also designated SEQ ID:384.

Another function of VGAM49 is therefore inhibition of DKFZP56611024 (Accession XM_{—}046506). Accordingly, utilities of VGAM49 include diagnosis, prevention and treatment of diseases and clinical conditions associated with DKFZP56611024. EP164 (Accession XM_{—}170997) is another VGAM49 host target gene. EP164 BINDING SITE is HOST TARGET binding site found in the 3′ untranslated region of mRNA encoded by EP164, corresponding to a HOST TARGET binding site such as BINDING SITE I, BINDING SITE II or BINDING SITE III. Table 2 illustrates the complementarity of the nucleotide sequences of EP164 BINDING SITE, designated SEQ ID:3681, to the nucleotide sequence of VGAM49 RNA, herein designated VGAM RNA, also designated SEQ ID:384.

Another function of VGAM49 is therefore inhibition of EP164 (Accession XM_{—}170997). Accordingly, utilities of VGAM49 include diagnosis, prevention and treatment of diseases and clinical conditions associated with EP164. FLJ00012 (Accession XM_{—}058426) is another VGAM49 host target gene. FLJ00012 BINDING SITE is HOST TARGET binding site found in the 3′ untranslated region of mRNA encoded by FLJ00012, corresponding to a HOST TARGET binding site such as BINDING SITE I, BINDING SITE II or BINDING SITE III. Table 2 illustrates the complementarity of the nucleotide sequences of FLJ00012 BINDING SITE, designated SEQ ID:2985, to the nucleotide sequence of VGAM49 RNA, herein designated VGAM RNA, also designated SEQ ID:384.

Another function of VGAM49 is therefore inhibition of FLJ00012 (Accession XM_{—}058426). Accordingly, utilities of VGAM49 include diagnosis, prevention and treatment of diseases and clinical conditions associated with FLJ00012. FLJ10460 (Accession NM_{—}018097) is another VGAM49 host target gene. FLJ10460 BINDING SITE is HOST TARGET binding site found in the 3′ untranslated region of mRNA encoded by FLJ10460, corresponding to a HOST TARGET binding site such as BINDING SITE I, BINDING SITE II or BINDING SITE III. Table 2 illustrates the complementarity of the nucleotide sequences of FLJ10460 BINDING SITE, designated SEQ ID:1778, to the nucleotide sequence of VGAM49 RNA, herein designated VGAM RNA, also designated SEQ ID:384.

Another function of VGAM49 is therefore inhibition of FLJ10460 (Accession NM_{—}018097). Accordingly, utilities of VGAM49 include diagnosis, prevention and treatment of diseases and clinical conditions associated with FLJ10460. FLJ10687 (Accession NM_{—}018178) is another VGAM49 host target gene. FLJ10687 BINDING SITE is HOST TARGET binding site found in the 3′ untranslated region of mRNA encoded by FLJ10687, corresponding to a HOST TARGET binding site such as BINDING SITE I, BINDING SITE II or BINDING SITE III. Table 2 illustrates the complementarity of the nucleotide sequences of FLJ10687 BINDING SITE, designated SEQ ID:1793, to the nucleotide sequence of VGAM49 RNA, herein designated VGAM RNA, also designated SEQ ID:384.

Another function of VGAM49 is therefore inhibition of FLJ10687 (Accession NM_{—}018178). Accordingly, utilities of VGAM49 include diagnosis, prevention and treatment of diseases and clinical conditions associated with FLJ10687. FLJ10738 (Accession NM_{—}018199) is another VGAM49 host target gene. FLJ10738 BINDING SITE is HOST TARGET binding site found in the 3′ untranslated region of mRNA encoded by FLJ10738, corresponding to a HOST TARGET binding site such as BINDING SITE I, BINDING SITE II or BINDING SITE III. Table 2 illustrates the complementarity of the nucleotide sequences of FLJ10738 BINDING SITE, designated SEQ ID:1799, to the nucleotide sequence of VGAM49 RNA, herein designated VGAM RNA, also designated SEQ ID:384.

Another function of VGAM49 is therefore inhibition of FLJ10738 (Accession NM_{—}018199). Accordingly, utilities of VGAM49 include diagnosis, prevention and treatment of diseases and clinical conditions associated with FLJ10738. FLJ13158 (Accession NM_{—}024909) is another VGAM49 host target gene. FLJ13158 BINDING SITE is HOST TARGET binding site found in the 3′ untranslated region of mRNA encoded by FLJ13158, corresponding to a HOST TARGET binding site such as BINDING SITE I, BINDING SITE II or BINDING SITE III. Table 2 illustrates the complementarity of the nucleotide sequences of FLJ13158 BINDING SITE, designated SEQ ID:2109, to the nucleotide sequence of VGAM49 RNA, herein designated VGAM RNA, also designated SEQ ID:384.

Another function of VGAM49 is therefore inhibition of FLJ13158 (Accession NM_{—}024909). Accordingly, utilities of VGAM49 include diagnosis, prevention and treatment of diseases and clinical conditions associated with FLJ13158. FLJ20294 (Accession NM_{—}017749) is another VGAM49 host target gene. FLJ20294 BINDING SITE is HOST TARGET binding site found in the 5′ untranslated region of mRNA encoded by FLJ20294, corresponding to a HOST TARGET binding site such as BINDING SITE I, BINDING SITE II or BINDING SITE III. Table 2 illustrates the complementarity of the nucleotide sequences of FLJ20294 BINDING SITE, designated SEQ ID:1747, to the nucleotide sequence of VGAM49 RNA, herein designated VGAM RNA, also designated SEQ ID:384.

Another function of VGAM49 is therefore inhibition of FLJ20294 (Accession NM_{—}017749). Accordingly, utilities of VGAM49 include diagnosis, prevention and treatment of diseases and clinical conditions associated with FLJ20294. FLJ21657 (Accession NM_{—}022483) is another VGAM49 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID:1991, to the nucleotide sequence of VGAM49 RNA, herein designated VGAM RNA, also designated SEQ ID:384.

Another function of VGAM49 is therefore inhibition of FLJ21657 (Accession NM_{—}022483). Accordingly, utilities of VGAM49 include diagnosis, prevention and treatment of diseases and clinical conditions associated with FLJ21657. GT650 (Accession NM_{—}052851) is another VGAM49 host target gene. GT650 BINDING SITE is HOST TARGET binding site found in the 3′ untranslated region of mRNA encoded by GT650, corresponding to a HOST TARGET binding site such as BINDING SITE I, BINDING SITE II or BINDING SITE III. Table 2 illustrates the complementarity of the nucleotide sequences of GT650 BINDING SITE, designated SEQ ID:2341, to the nucleotide sequence of VGAM49 RNA, herein designated VGAM RNA, also designated SEQ ID:384.

Another function of VGAM49 is therefore inhibition of GT650 (Accession NM_{—}052851). Accordingly, utilities of VGAM49 include diagnosis, prevention and treatment of diseases and clinical conditions associated with GT650. Hairy/enhancer-of-split Related with YRPW Motif 2 (HEY2, Accession NM_{—}012259) is another VGAM49 host target gene. HEY2 BINDING SITE is HOST TARGET binding site found in the 3′ untranslated region of mRNA encoded by HEY2, corresponding to a HOST TARGET binding site such as BINDING SITE I, BINDING SITE II or BINDING SITE III. Table 2 illustrates the complementarity of the nucleotide sequences of HEY2 BINDING SITE, designated SEQ ID: 1420, to the nucleotide sequence of VGAM49 RNA, herein designated VGAM RNA, also designated SEQ ID:384.

Another function of VGAM49 is therefore inhibition of Hairy/enhancer-of-split Related with YRPW Motif 2 (HEY2, Accession NM_{—}012259). Accordingly, utilities of VGAM49 include diagnosis, prevention and treatment of diseases and clinical conditions associated with HEY2. Hypermethylated In Cancer 2 (HIC2, Accession XM_{—}036937) is another VGAM49 host target gene. HIC2 BINDING SITE is HOST TARGET binding site found in the 3′ untranslated region of mRNA encoded by HIC2, corresponding to a HOST TARGET binding site such as BINDING SITE I, BINDING SITE II or BINDING SITE III. Table 2 illustrates the complementarity of the nucleotide sequences of HIC2 BINDING SITE, designated SEQ ID:2717, to the nucleotide sequence of VGAM49 RNA, herein designated VGAM RNA, also designated SEQ ID:384.

Another function of VGAM49 is therefore inhibition of Hypermethylated In Cancer 2 (HIC2, Accession XM_{—}036937). Accordingly, utilities of VGAM49 include diagnosis, prevention and treatment of diseases and clinical conditions associated with HIC2. HOOK3 (Accession NM_{—}032410) is another VGAM49 host target gene. HOOK3 BINDING SITE is HOST TARGET binding site found in the 3′ untranslated region of mRNA encoded by HOOK3, corresponding to a HOST TARGET binding site such as BINDING SITE I, BINDING SITE II or BINDING SITE III. Table 2 illustrates the complementarity of the nucleotide sequences of HOOK3 BINDING SITE, designated SEQ ID:2249, to the nucleotide sequence of VGAM49 RNA, herein designated VGAM RNA, also designated SEQ ID:384.

Another function of VGAM49 is therefore inhibition of HOOK3 (Accession NM_{—}032410). Accordingly, utilities of VGAM49 include diagnosis, prevention and treatment of diseases and clinical conditions associated with HOOK3. Interleukin 1 Receptor Accessory Protein-like 1 (IL1RAPL1, Accession NM_{—}014271) is another VGAM49 host target gene. IL1RAPL1 BINDING SITE is HOST TARGET binding site found in the 5′ untranslated region of mRNA encoded by IL1RAPL1, corresponding to a HOST TARGET binding site such as BINDING SITE I, BINDING SITE II or BINDING SITE III. Table 2 illustrates the complementarity of the nucleotide sequences of IL1RAPL1 BINDING SITE, designated SEQ ID: 1488, to the nucleotide sequence of VGAM49 RNA, herein designated VGAM RNA, also designated SEQ ID:384.

Another function of VGAM49 is therefore inhibition of Interleukin 1 Receptor Accessory Protein-like 1 (IL1RAPL1, Accession NM_{—}014271). Accordingly, utilities of VGAM49 include diagnosis, prevention and treatment of diseases and clinical conditions associated with IL1RAPL1. KALI (Accession NM_{—}052931) is another VGAM49 host target gene. KALI BINDING SITE is HOST TARGET binding site found in the 3′ untranslated region of mRNA encoded by KALI, corresponding to a HOST TARGET binding site such as BINDING SITE I, BINDING SITE II or BINDING SITE III. Table 2 illustrates the complementarity of the nucleotide sequences of KALI BINDING SITE, designated SEQ ID:2347, to the nucleotide sequence of VGAM49 RNA, herein designated VGAM RNA, also designated SEQ ID:384.

Another function of VGAM49 is therefore inhibition of KALI (Accession NM_{—}052931). Accordingly, utilities of VGAM49 include diagnosis, prevention and treatment of diseases and clinical conditions associated with KALI. KIAA0077 (Accession XM_{—}040158) is another VGAM49 host target gene. KIAA0077 BINDING SITE is HOST TARGET binding site found in the 3′ untranslated region of mRNA encoded by KIAA0077, corresponding to a HOST TARGET binding site such as BINDING SITE I, BINDING SITE II or BINDING SITE III. Table 2 illustrates the complementarity of the nucleotide sequences of KIAA0077 BINDING SITE, designated SEQ ID:2773, to the nucleotide sequence of VGAM49 RNA, herein designated VGAM RNA, also designated SEQ ID:384.

Another function of VGAM49 is therefore inhibition of KIAA0077 (Accession XM_{—}040158). Accordingly, utilities of VGAM49 include diagnosis, prevention and treatment of diseases and clinical conditions associated with KIAA0077. KIAA0089 (Accession XM_{—}046056) is another VGAM49 host target gene. KIAA0089 BINDING SITE is HOST TARGET binding site found in the 3′ untranslated region of mRNA encoded by KIAA0089, corresponding to a HOST TARGET binding site such as BINDING SITE I, BINDING SITE II or BINDING SITE III. Table 2 illustrates the complementarity of the nucleotide sequences of KIAA0089 BINDING SITE, designated SEQ ID:2873, to the nucleotide sequence of VGAM49 RNA, herein designated VGAM RNA, also designated SEQ ID:384.

Another function of VGAM49 is therefore inhibition of KIAA0089 (Accession XM_{—}046056). Accordingly, utilities of VGAM49 include diagnosis, prevention and treatment of diseases and clinical conditions associated with KIAA0089. KIAA0286 (Accession XM_{—}043118) is another VGAM49 host target gene. KIAA0286 BINDING SITE is HOST TARGET binding site found in the 3′ untranslated region of mRNA encoded by KIAA0286, corresponding to a HOST TARGET binding site such as BINDING SITE I, BINDING SITE II or BINDING SITE III. Table 2 illustrates the complementarity of the nucleotide sequences of KIAA0286 BINDING SITE, designated SEQ ID:2814, to the nucleotide sequence of VGAM49 RNA, herein designated VGAM RNA, also designated SEQ ID:384.

Another function of VGAM49 is therefore inhibition of KIAA0286 (Accession XM_{—}043118). Accordingly, utilities of VGAM49 include diagnosis, prevention and treatment of diseases and clinical conditions associated with KIAA0286. KIAA0295 (Accession XM_{—}042833) is another VGAM49 host target gene. KIAA0295 BINDING SITE is HOST TARGET binding site found in the 3′ untranslated region of mRNA encoded by KIAA0295, corresponding to a HOST TARGET binding site such as BINDING SITE I, BINDING SITE II or BINDING SITE III. Table 2 illustrates the complementarity of the nucleotide sequences of KIAA0295 BINDING SITE, designated SEQ ID:2808, to the nucleotide sequence of VGAM49 RNA, herein designated VGAM RNA, also designated SEQ ID:384.

Another function of VGAM49 is therefore inhibition of KIAA0295 (Accession XM_{—}042833). Accordingly, utilities of VGAM49 include diagnosis, prevention and treatment of diseases and clinical conditions associated with KIAA0295. KIAA0342 (Accession XM_{—}047357) is another VGAM49 host target gene. KIAA0342 BINDING SITE is HOST TARGET binding site found in the 3′ untranslated region of mRNA encoded by KIAA0342, corresponding to a HOST TARGET binding site such as BINDING SITE I, BINDING SITE II or BINDING SITE III. Table 2 illustrates the complementarity of the nucleotide sequences of KIAA0342 BINDING SITE, designated SEQ ID:2890, to the nucleotide sequence of VGAM49 RNA, herein designated VGAM RNA, also designated SEQ ID:384.

Another function of VGAM49 is therefore inhibition of KIAA0342 (Accession XM_{—}047357). Accordingly, utilities of VGAM49 include diagnosis, prevention and treatment of diseases and clinical conditions associated with KIAA0342. KIAA0564 (Accession XM_{—}038664) is another VGAM49 host target gene. KIAA0564 BINDING SITE is HOST TARGET binding site found in the 3′ untranslated region of mRNA encoded by KIAA0564, corresponding to a HOST TARGET binding site such as BINDING SITE I, BINDING SITE II or BINDING SITE III. Table 2 illustrates the complementarity of the nucleotide sequences of KIAA0564 BINDING SITE, designated SEQ ID:2743, to the nucleotide sequence of VGAM49 RNA, herein designated VGAM RNA, also designated SEQ ID:384.

Another function of VGAM49 is therefore inhibition of KIAA0564 (Accession XM_{—}038664). Accordingly, utilities of VGAM49 include diagnosis, prevention and treatment of diseases and clinical conditions associated with KIAA0564. KIAA0630 (Accession XM_{—}114729) is another VGAM49 host target gene. KIAA0630 BINDING SITE is HOST TARGET binding site found in the 3′ untranslated region of mRNA encoded by KIAA0630, corresponding to a HOST TARGET binding site such as BINDING SITE I, BINDING SITE II or BINDING SITE III. Table 2 illustrates the complementarity of the nucleotide sequences of KIAA0630 BINDING SITE, designated SEQ ID:3465, to the nucleotide sequence of VGAM49 RNA, herein designated VGAM RNA, also designated SEQ ID:384.

Another function of VGAM49 is therefore inhibition of KIAA0630 (Accession XM_{—}114729). Accordingly, utilities of VGAM49 include diagnosis, prevention and treatment of diseases and clinical conditions associated with KIAA0630. KIAA0662 (Accession XM_{—}088539) is another VGAM49 host target gene. KIAA0662 BINDING SITE is HOST TARGET binding site found in the 3′ untranslated region of mRNA encoded by KIAA0662, corresponding to a HOST TARGET binding site such as BINDING SITE I, BINDING SITE II or BINDING SITE III. Table 2 illustrates the complementarity of the nucleotide sequences of KIAA0662 BINDING SITE, designated SEQ ID:3215, to the nucleotide sequence of VGAM49 RNA, herein designated VGAM RNA, also designated SEQ ID:384.

Another function of VGAM49 is therefore inhibition of KIAA0662 (Accession XM_{—}088539). Accordingly, utilities of VGAM49 include diagnosis, prevention and treatment of diseases and clinical conditions associated with KIAA0662. KIAA0830 (Accession XM_{—}045759) is another VGAM49 host target gene. KIAA0830 BINDING SITE is HOST TARGET binding site found in the 3′ untranslated region of mRNA encoded by KIAA0830, corresponding to a HOST TARGET binding site such as BINDING SITE I, BINDING SITE II or BINDING SITE III. Table 2 illustrates the complementarity of the nucleotide sequences of KIAA0830 BINDING SITE, designated SEQ ID:2866, to the nucleotide sequence of VGAM49 RNA, herein designated VGAM RNA, also designated SEQ ID:384.

Another function of VGAM49 is therefore inhibition of KIAA0830 (Accession XM_{—}045759). Accordingly, utilities of VGAM49 include diagnosis, prevention and treatment of diseases and clinical conditions associated with KIAA0830. KIAA0964 (Accession NM_{—}014902) is another VGAM49 host target gene. KIAA0964 BINDING SITE is HOST TARGET binding site found in the 3′ untranslated region of mRNA encoded by KIAA0964, corresponding to a HOST TARGET binding site such as BINDING SITE I, BINDING SITE II or BINDING SITE III. Table 2 illustrates the complementarity of the nucleotide sequences of KIAA0964 BINDING SITE, designated SEQ ID:1579, to the nucleotide sequence of VGAM49 RNA, herein designated VGAM RNA, also designated SEQ ID:384.

Another function of VGAM49 is therefore inhibition of KIAA0964 (Accession NM_{—}014902). Accordingly, utilities of VGAM49 include diagnosis, prevention and treatment of diseases and clinical conditions associated with KIAA0964. KIAA1076 (Accession XM_{—}037523) is another VGAM49 host target gene. KIAA1076 BINDING SITE is HOST TARGET binding site found in the 3′ untranslated region of mRNA encoded by KIAA1076, corresponding to a HOST TARGET binding site such as BINDING SITE I, BINDING SITE II or BINDING SITE III. Table 2 illustrates the complementarity of the nucleotide sequences of KIAA1076 BINDING SITE, designated SEQ ID:2723, to the nucleotide sequence of VGAM49 RNA, herein designated VGAM RNA, also designated SEQ ID:384.

Another function of VGAM49 is therefore inhibition of KIAA1076 (Accession XM_{—}037523). Accordingly, utilities of VGAM49 include diagnosis, prevention and treatment of diseases and clinical conditions associated with KIAA1076. KIAA1181 (Accession XM_{—}043340) is another VGAM49 host target gene. KIAA1181 BINDING SITE is HOST TARGET binding site found in the 3′ untranslated region of mRNA encoded by KIAA1181, corresponding to a HOST TARGET binding site such as BINDING SITE I, BINDING SITE II or BINDING SITE III. Table 2 illustrates the complementarity of the nucleotide sequences of KIAA1181 BINDING SITE, designated SEQ ID:2817, to the nucleotide sequence of VGAM49 RNA, herein designated VGAM RNA, also designated SEQ ID:384.

Another function of VGAM49 is therefore inhibition of KIAA1181 (Accession XM_{—}043340). Accordingly, utilities of VGAM49 include diagnosis, prevention and treatment of diseases and clinical conditions associated with KIAA1181. KIAA1319 (Accession NM_{—}020770) is another VGAM49 host target gene. KIAA1319 BINDING SITE is HOST TARGET binding site found in the 3′ untranslated region of mRNA encoded by KIAA1319, corresponding to a HOST TARGET binding site such as BINDING SITE I, BINDING SITE II or BINDING SITE III. Table 2 illustrates the complementarity of the nucleotide sequences of KIAA1319 BINDING SITE, designated SEQ ID:1925, to the nucleotide sequence of VGAM49 RNA, herein designated VGAM RNA, also designated SEQ ID:384.

Another function of VGAM49 is therefore inhibition of KIAA1319 (Accession NM_{—}020770). Accordingly, utilities of VGAM49 include diagnosis, prevention and treatment of diseases and clinical conditions associated with KIAA1319. KIAA1522 (Accession XM_{—}036299) is another VGAM49 host target gene. KIAA1522 BINDING SITE is HOST TARGET binding site found in the 3′ untranslated region of mRNA encoded by KIAA1522, corresponding to a HOST TARGET binding site such as BINDING SITE I, BINDING SITE II or BINDING SITE III. Table 2 illustrates the complementarity of the nucleotide sequences of KIAA1522 BINDING SITE, designated SEQ ID:2708, to the nucleotide sequence of VGAM49 RNA, herein designated VGAM RNA, also designated SEQ ID:384.

Another function of VGAM49 is therefore inhibition of KIAA1522 (Accession XM_{—}036299). Accordingly, utilities of VGAM49 include diagnosis, prevention and treatment of diseases and clinical conditions associated with KIAA1522. KIAA1906 (Accession XM_{—}055095) is another VGAM49 host target gene. KIAA1906 BINDING SITE is HOST TARGET binding site found in the 3′ untranslated region of mRNA encoded by KIAA1906, corresponding to a HOST TARGET binding site such as BINDING SITE I, BINDING SITE II or BINDING SITE III. Table 2 illustrates the complementarity of the nucleotide sequences of KIAA1906 BINDING SITE, designated SEQ ID:2971, to the nucleotide sequence of VGAM49 RNA, herein designated VGAM RNA, also designated SEQ ID:384.

Another function of VGAM49 is therefore inhibition of KIAA1906 (Accession XM_{—}055095). Accordingly, utilities of VGAM49 include diagnosis, prevention and treatment of diseases and clinical conditions associated with KIAA1906. Mitogen-activated Protein Kinase 6 (MAPK6, Accession NM_{—}002748) is another VGAM49 host target gene. MAPK6 BINDING SITE is HOST TARGET binding site found in the 5′ untranslated region of mRNA encoded by MAPK6, corresponding to a HOST TARGET binding site such as BINDING SITE I, BINDING SITE II or BINDING SITE III. Table 2 illustrates the complementarity of the nucleotide sequences of MAPK6 BINDING SITE, designated SEQ ID:952, to the nucleotide sequence of VGAM49 RNA, herein designated VGAM RNA, also designated SEQ ID:384.

Another function of VGAM49 is therefore inhibition of Mitogen-activated Protein Kinase 6 (MAPK6, Accession NM_{—}002748). Accordingly, utilities of VGAM49 include diagnosis, prevention and treatment of diseases and clinical conditions associated with MAPK6. MGC13138 (Accession NM_{—}033410) is another VGAM49 host target gene. MGC13138 BINDING SITE is HOST TARGET binding site found in the 3′ untranslated region of mRNA encoded by MGC13138, corresponding to a HOST TARGET binding site such as BINDING SITE I, BINDING SITE II or BINDING SITE III. Table 2 illustrates the complementarity of the nucleotide sequences of MGC13138 BINDING SITE, designated SEQ ID:2332, to the nucleotide sequence of VGAM49 RNA, herein designated VGAM RNA, also designated SEQ ID:384.

Another function of VGAM49 is therefore inhibition of MGC13138 (Accession NM_{—}033410). Accordingly, utilities of VGAM49 include diagnosis, prevention and treatment of diseases and clinical conditions associated with MGC13138. MGC2742 (Accession NM_{—}023938) is another VGAM49 host target gene. MGC2742 BINDING SITE is HOST TARGET binding site found in the 5′ untranslated region of mRNA encoded by MGC2742, corresponding to a HOST TARGET binding site such as BINDING SITE I, BINDING SITE II or BINDING SITE III. Table 2 illustrates the complementarity of the nucleotide sequences of MGC2742 BINDING SITE, designated SEQ ID:2040, to the nucleotide sequence of VGAM49 RNA, herein designated VGAM RNA, also designated SEQ ID:384.

Another function of VGAM49 is therefore inhibition of MGC2742 (Accession NM_{—}023938). Accordingly, utilities of VGAM49 include diagnosis, prevention and treatment of diseases and clinical conditions associated with MGC2742. MGC32043 (Accession NM_{—}144582) is another VGAM49 host target gene. MGC32043 BINDING SITE is HOST TARGET binding site found in the 3′ untranslated region of mRNA encoded by MGC32043, corresponding to a HOST TARGET binding site such as BINDING SITE I, BINDING SITE II or BINDING SITE III. Table 2 illustrates the complementarity of the nucleotide sequences of MGC32043 BINDING SITE, designated SEQ ID:2486, to the nucleotide sequence of VGAM49 RNA, herein designated VGAM RNA, also designated SEQ ID:384.

Another function of VGAM49 is therefore inhibition of MGC32043 (Accession NM_{—}144582). Accordingly, utilities of VGAM49 include diagnosis, prevention and treatment of diseases and clinical conditions associated with MGC32043. Nuclear Factor of Activated T-cells 5, Tonicity-responsive (NFAT5, Accession NM_{—}138714) is another VGAM49 host target gene. NFAT5 BINDING SITE is HOST TARGET binding site found in the 3′ untranslated region of mRNA encoded by NFAT5, corresponding to a HOST TARGET binding site such as BINDING SITE I, BINDING SITE II or BINDING SITE III. Table 2 illustrates the complementarity of the nucleotide sequences of NFAT5 BINDING SITE, designated SEQ ID:2452, to the nucleotide sequence of VGAM49 RNA, herein designated VGAM RNA, also designated SEQ ID:384.

Another function of VGAM49 is therefore inhibition of Nuclear Factor of Activated T-cells 5, Tonicity-responsive (NFAT5, Accession NM_{—}138714). Accordingly, utilities of VGAM49 include diagnosis, prevention and treatment of diseases and clinical conditions associated with NFAT5. Oxysterol Binding Protein-like 8 (OSBPL8, Accession NM_{—}020841) is another VGAM49 host target gene. OSBPL8 BINDING SITE is HOST TARGET binding site found in the 3′ untranslated region of mRNA encoded by OSBPL8, corresponding to a HOST TARGET binding site such as BINDING SITE I, BINDING SITE II or BINDING SITE III. Table 2 illustrates the complementarity of the nucleotide sequences of OSBPL8 BINDING SITE, designated SEQ ID:1929, to the nucleotide sequence of VGAM49 RNA, herein designated VGAM RNA, also designated SEQ ID:384.

Another function of VGAM49 is therefore inhibition of Oxysterol Binding Protein-like 8 (OSBPL8, Accession NM_{—}020841). Accordingly, utilities of VGAM49 include diagnosis, prevention and treatment of diseases and clinical conditions associated with OSBPL8. RAB10, Member RAS Oncogene Family (RAB10, Accession XM_{—}097979) is another VGAM49 host target gene. RAB10 BINDING SITE is HOST TARGET binding site found in the 3′ untranslated region of mRNA encoded by RAB10, corresponding to a HOST TARGET binding site such as BINDING SITE I, BINDING SITE II or BINDING SITE III. Table 2 illustrates the complementarity of the nucleotide sequences of RAB10 BINDING SITE, designated SEQ ID:3337, to the nucleotide sequence of VGAM49 RNA, herein designated VGAM RNA, also designated SEQ ID:384.

Another function of VGAM49 is therefore inhibition of RAB10, Member RAS Oncogene Family (RAB10, Accession XM_{—}097979). Accordingly, utilities of VGAM49 include diagnosis, prevention and treatment of diseases and clinical conditions associated with RAB10. RAN Binding Protein 1 (RANBP1, Accession NM_{—}002882) is another VGAM49 host target gene. RANBP1 BINDING SITE is HOST TARGET binding site found in the 3′ untranslated region of mRNA encoded by RANBP1, corresponding to a HOST TARGET binding site such as BINDING SITE I, BINDING SITE II or BINDING SITE III. Table 2 illustrates the complementarity of the nucleotide sequences of RANBP1 BINDING SITE, designated SEQ ID:969, to the nucleotide sequence of VGAM49 RNA, herein designated VGAM RNA, also designated SEQ ID:384.

Another function of VGAM49 is therefore inhibition of RAN Binding Protein 1 (RANBP1, Accession NM_{—}002882). Accordingly, utilities of VGAM49 include diagnosis, prevention and treatment of diseases and clinical conditions associated with RANBP1. Ras Association (RalGDS/AF-6) Domain Family 2 (RASSF2, Accession NM_{—}014737) is another VGAM49 host target gene. RASSF2 BINDING SITE is HOST TARGET binding site found in the 3′ untranslated region of mRNA encoded by RASSF2, corresponding to a HOST TARGET binding site such as BINDING SITE I, BINDING SITE II or BINDING SITE III. Table 2 illustrates the complementarity of the nucleotide sequences of RASSF2 BINDING SITE, designated SEQ ID:1537, to the nucleotide sequence of VGAM49 RNA, herein designated VGAM RNA, also designated SEQ ID:384.

Another function of VGAM49 is therefore inhibition of Ras Association (RalGDS/AF-6) Domain Family 2 (RASSF2, Accession NM_{—}014737). Accordingly, utilities of VGAM49 include diagnosis, prevention and treatment of diseases and clinical conditions associated with RASSF2. Serine/threonine Kinase 22D (spermiogenesis associated) (STK22D, Accession NM_{—}032028) is another VGAM49 host target gene. STK22D BINDING SITE is HOST TARGET binding site found in the 3′ untranslated region of mRNA encoded by STK22D, corresponding to a HOST TARGET binding site such as BINDING SITE I, BINDING SITE II or BINDING SITE III. Table 2 illustrates the complementarity of the nucleotide sequences of STK22D BINDING SITE, designated SEQ ID:2226, to the nucleotide sequence of VGAM49 RNA, herein designated VGAM RNA, also designated SEQ ID:384.

Another function of VGAM49 is therefore inhibition of Serine/threonine Kinase 22D (spermiogenesis associated) (STK22D, Accession NM_{—}032028). Accordingly, utilities of VGAM49 include diagnosis, prevention and treatment of diseases and clinical conditions associated with STK22D. Synaptojanin 2 (SYNJ2, Accession XM_{—}029746) is another VGAM49 host target gene. SYNJ2 BINDING SITE is HOST TARGET binding site found in the 3′ untranslated region of mRNA encoded by SYNJ2, corresponding to a HOST TARGET binding site such as BINDING SITE I, BINDING SITE II or BINDING SITE III. Table 2 illustrates the complementarity of the nucleotide sequences of SYNJ2 BINDING SITE, designated SEQ ID:2606, to the nucleotide sequence of VGAM49 RNA, herein designated VGAM RNA, also designated SEQ ID:384.

Another function of VGAM49 is therefore inhibition of Synaptojanin 2 (SYNJ2, Accession XM_{—}029746). Accordingly, utilities of VGAM49 include diagnosis, prevention and treatment of diseases and clinical conditions associated with SYNJ2. TBLR1 (Accession NM_{—}024665) is another VGAM49 host target gene. TBLR1 BINDING SITE is HOST TARGET binding site found in the 3′ untranslated region of mRNA encoded by TBLR1, corresponding to a HOST TARGET binding site such as BINDING SITE I, BINDING SITE II or BINDING SITE III. Table 2 illustrates the complementarity of the nucleotide sequences of TBLR1 BINDING SITE, designated SEQ ID:2079, to the nucleotide sequence of VGAM49 RNA, herein designated VGAM RNA, also designated SEQ ID:384.

Another function of VGAM49 is therefore inhibition of TBLR1 (Accession NM_{—}024665). Accordingly, utilities of VGAM49 include diagnosis, prevention and treatment of diseases and clinical conditions associated with TBLR1. Transmembrane 9 Super family Member 1 (TM9SF1, Accession NM_{—}006405) is another VGAM49 host target gene. TM9SF1 BINDING SITE is HOST TARGET binding site found in the 3′ untranslated region of mRNA encoded by TM9SF1, corresponding to a HOST TARGET binding site such as BINDING SITE I, BINDING SITE II or BINDING SITE III. Table 2 illustrates the complementarity of the nucleotide sequences of TM9SF1 BINDING SITE, designated SEQ ID:1296, to the nucleotide sequence of VGAM49 RNA, herein designated VGAM RNA, also designated SEQ ID:384.

Another function of VGAM49 is therefore inhibition of Transmembrane 9 Super family Member 1 (TM9SF1, Accession NM_{—}006405). Accordingly, utilities of VGAM49 include diagnosis, prevention and treatment of diseases and clinical conditions associated with TM9SF1. Unc-51-like Kinase 2 (C. elegans) (ULK2, Accession NM_{—}014683) is another VGAM49 host target gene. ULK2 BINDING SITE is HOST TARGET binding site found in the 3′ untranslated region of mRNA encoded by ULK2, corresponding to a HOST TARGET binding site such as BINDING SITE I, BINDING SITE II or BINDING SITE III. Table 2 illustrates the complementarity of the nucleotide sequences of ULK2 BINDING SITE, designated SEQ ID:1523, to the nucleotide sequence of VGAM49 RNA, herein designated VGAM RNA, also designated SEQ ID:384.

Another function of VGAM49 is therefore inhibition of Unc-51-like Kinase 2 (C. elegans) (ULK2, Accession NM_{—}014683). Accordingly, utilities of VGAM49 include diagnosis, prevention and treatment of diseases and clinical conditions associated with ULK2. Unc-5 Homolog D (C. elegans) (UNC5D, Accession NM_{—}080872) is another VGAM49 host target gene. UNC5D BINDING SITE is HOST TARGET binding site found in the 3′ untranslated region of mRNA encoded by UNC5D, corresponding to a HOST TARGET binding site such as BINDING SITE I, BINDING SITE II or BINDING SITE III. Table 2 illustrates the complementarity of the nucleotide sequences of UNC5D BINDING SITE, designated SEQ ID:2393, to the nucleotide sequence of VGAM49 RNA, herein designated VGAM RNA, also designated SEQ ID:384.

Another function of VGAM49 is therefore inhibition of Unc-5 Homolog D (C. elegans) (UNC5D, Accession NM_{—}080872). Accordingly, utilities of VGAM49 include diagnosis, prevention and treatment of diseases and clinical conditions associated with UNC5D. LOC121219 (Accession XM_{—}058544) is another VGAM49 host target gene. LOC121219 BINDING SITE is HOST TARGET binding site found in the 5′ untranslated region of mRNA encoded by LOC121219, corresponding to a HOST TARGET binding site such as BINDING SITE I, BINDING SITE II or BINDING SITE III. Table 2 illustrates the complementarity of the nucleotide sequences of LOC121219 BINDING SITE, designated SEQ ID:2988, to the nucleotide sequence of VGAM49 RNA, herein designated VGAM RNA, also designated SEQ ID:384.

Another function of VGAM49 is therefore inhibition of LOC121219 (Accession XM_{—}058544). Accordingly, utilities of VGAM49 include diagnosis, prevention and treatment of diseases and clinical conditions associated with LOC121219. LOC128077 (Accession XM_{—}059208) is another VGAM49 host target gene. LOC128077 BINDING SITE is HOST TARGET binding site found in the 3′ untranslated region of mRNA encoded by LOC128077, corresponding to a HOST TARGET binding site such as BINDING SITE I, BINDING SITE II or BINDING SITE III. Table 2 illustrates the complementarity of the nucleotide sequences of LOC128077 BINDING SITE, designated SEQ ID:3006, to the nucleotide sequence of VGAM49 RNA, herein designated VGAM RNA, also designated SEQ ID:384.

Another function of VGAM49 is therefore inhibition of LOC128077 (Accession XM_{—}059208). Accordingly, utilities of VGAM49 include diagnosis, prevention and treatment of diseases and clinical conditions associated with LOC128077. LOC143451 (Accession XM_{—}084521) is another VGAM49 host target gene. LOC143451 BINDING SITE is HOST TARGET binding site found in the 3′ untranslated region of mRNA encoded by LOC143451, corresponding to a HOST TARGET binding site such as BINDING SITE I, BINDING SITE II or BINDING SITE III. Table 2 illustrates the complementarity of the nucleotide sequences of LOC143451 BINDING SITE, designated SEQ ID:3058, to the nucleotide sequence of VGAM49 RNA, herein designated VGAM RNA, also designated SEQ ID:384.

Another function of VGAM49 is therefore inhibition of LOC143451 (Accession XM_{—}084521). Accordingly, utilities of VGAM49 include diagnosis, prevention and treatment of diseases and clinical conditions associated with LOC143451. LOC145125 (Accession XM_{—}085025) is another VGAM49 host target gene. LOC145125 BINDING SITE is HOST TARGET binding site found in the 3′ untranslated region of mRNA encoded by LOC145125, corresponding to a HOST TARGET binding site such as BINDING SITE I, BINDING SITE II or BINDING SITE III. Table 2 illustrates the complementarity of the nucleotide sequences of LOC145125 BINDING SITE, designated SEQ ID:3075, to the nucleotide sequence of VGAM49 RNA, herein designated VGAM RNA, also designated SEQ ID:384.

Another function of VGAM49 is therefore inhibition of LOC145125 (Accession XM_{—}085025). Accordingly, utilities of VGAM49 include diagnosis, prevention and treatment of diseases and clinical conditions associated with LOC145125. LOC149351 (Accession XM_{—}086503) is another VGAM49 host target gene. LOC149351 BINDING SITE is HOST TARGET binding site found in the 3′ untranslated region of mRNA encoded by LOC149351, corresponding to a HOST TARGET binding site such as BINDING SITE I, BINDING SITE II or BINDING SITE III. Table 2 illustrates the complementarity of the nucleotide sequences of LOC149351 BINDING SITE, designated SEQ ID:3131, to the nucleotide sequence of VGAM49 RNA, herein designated VGAM RNA, also designated SEQ ID:384.

Another function of VGAM49 is therefore inhibition of LOC149351 (Accession XM_{—}086503). Accordingly, utilities of VGAM49 include diagnosis, prevention and treatment of diseases and clinical conditions associated with LOC149351. LOC149722 (Accession XM_{—}097709) is another VGAM49 host target gene. LOC149722 BINDING SITE is HOST TARGET binding site found in the 5′ untranslated region of mRNA encoded by LOC149722, corresponding to a HOST TARGET binding site such as BINDING SITE I, BINDING SITE II or BINDING SITE III. Table 2 illustrates the complementarity of the nucleotide sequences of LOC149722 BINDING SITE, designated SEQ ID:3312, to the nucleotide sequence of VGAM49 RNA, herein designated VGAM RNA, also designated SEQ ID:384.

Another function of VGAM49 is therefore inhibition of LOC149722 (Accession XM_{—}097709). Accordingly, utilities of VGAM49 include diagnosis, prevention and treatment of diseases and clinical conditions associated with LOC149722. LOC149837 (Accession XM_{—}097747) is another VGAM49 host target gene. LOC149837 BINDING SITE is HOST TARGET binding site found in the 5′ untranslated region of mRNA encoded by LOC149837, corresponding to a HOST TARGET binding site such as BINDING SITE I, BINDING SITE II or BINDING SITE III. Table 2 illustrates the complementarity of the nucleotide sequences of LOC149837 BINDING SITE, designated SEQ ID:3318, to the nucleotide sequence of VGAM49 RNA, herein designated VGAM RNA, also designated SEQ ID:384.

Another function of VGAM49 is therefore inhibition of LOC149837 (Accession XM_{—}097747). Accordingly, utilities of VGAM49 include diagnosis, prevention and treatment of diseases and clinical conditions associated with LOC149837. LOC150054 (Accession XM_{—}097797) is another VGAM49 host target gene. LOC150054 BINDING SITE is HOST TARGET binding site found in the 3′ untranslated region of mRNA encoded by LOC150054, corresponding to a HOST TARGET binding site such as BINDING SITE I, BINDING SITE II or BINDING SITE III. Table 2 illustrates the complementarity of the nucleotide sequences of LOC150054 BINDING SITE, designated SEQ ID:3323, to the nucleotide sequence of VGAM49 RNA, herein designated VGAM RNA, also designated SEQ ID:384.

Another function of VGAM49 is therefore inhibition of LOC150054 (Accession XM_{—}097797). Accordingly, utilities of VGAM49 include diagnosis, prevention and treatment of diseases and clinical conditions associated with LOC150054. LOC150150 (Accession XM_{—}097820) is another VGAM49 host target gene. LOC150150 BINDING SITE is HOST TARGET binding site found in the 5′ untranslated region of mRNA encoded by LOC150150, corresponding to a HOST TARGET binding site such as BINDING SITE I, BINDING SITE II or BINDING SITE III. Table 2 illustrates the complementarity of the nucleotide sequences of LOC150150 BINDING SITE, designated SEQ ID:3324, to the nucleotide sequence of VGAM49 RNA, herein designated VGAM RNA, also designated SEQ ID:384.

Another function of VGAM49 is therefore inhibition of LOC150150 (Accession XM_{—}097820). Accordingly, utilities of VGAM49 include diagnosis, prevention and treatment of diseases and clinical conditions associated with LOC150150. LOC150225 (Accession XM_{—}097870) is another VGAM49 host target gene. LOC150225 BINDING SITE is HOST TARGET binding site found in the 3′ untranslated region of mRNA encoded by LOC150225, corresponding to a HOST TARGET binding site such as BINDING SITE I, BINDING SITE II or BINDING SITE III. Table 2 illustrates the complementarity of the nucleotide sequences of LOC150225 BINDING SITE, designated SEQ ID:3327, to the nucleotide sequence of VGAM49 RNA, herein designated VGAM RNA, also designated SEQ ID:384.

Another function of VGAM49 is therefore inhibition of LOC150225 (Accession XM_{—}097870). Accordingly, utilities of VGAM49 include diagnosis, prevention and treatment of diseases and clinical conditions associated with LOC150225. LOC152816 (Accession XM_{—}098270) is another VGAM49 host target gene. LOC152816 BINDING SITE is HOST TARGET binding site found in the 3′ untranslated region of mRNA encoded by LOC152816, corresponding to a HOST TARGET binding site such as BINDING SITE I, BINDING SITE II or BINDING SITE III. Table 2 illustrates the complementarity of the nucleotide sequences of LOC152816 BINDING SITE, designated SEQ ID:3364, to the nucleotide sequence of VGAM49 RNA, herein designated VGAM RNA, also designated SEQ ID:384.

Another function of VGAM49 is therefore inhibition of LOC152816 (Accession XM_{—}098270). Accordingly, utilities of VGAM49 include diagnosis, prevention and treatment of diseases and clinical conditions associated with LOC152816. LOC155038 (Accession XM_{—}088130) is another VGAM49 host target gene. LOC155038 BINDING SITE is HOST TARGET binding site found in the 3′ untranslated region of mRNA encoded by LOC155038, corresponding to a HOST TARGET binding site such as BINDING SITE I, BINDING SITE II or BINDING SITE III. Table 2 illustrates the complementarity of the nucleotide sequences of LOC155038 BINDING SITE, designated SEQ ID:3198, to the nucleotide sequence of VGAM49 RNA, herein designated VGAM RNA, also designated SEQ ID:384.

Another function of VGAM49 is therefore inhibition of LOC155038 (Accession XM_{—}088130). Accordingly, utilities of VGAM49 include diagnosis, prevention and treatment of diseases and clinical conditions associated with LOC155038. LOC158629 (Accession XM_{—}098972) is another VGAM49 host target gene. LOC158629 BINDING SITE is HOST TARGET binding site found in the 3′ untranslated region of mRNA encoded by LOC158629, corresponding to a HOST TARGET binding site such as BINDING SITE I, BINDING SITE II or BINDING SITE III. Table 2 illustrates the complementarity of the nucleotide sequences of LOC158629 BINDING SITE, designated SEQ ID:3394, to the nucleotide sequence of VGAM49 RNA, herein designated VGAM RNA, also designated SEQ ID:384.

Another function of VGAM49 is therefore inhibition of LOC158629 (Accession XM_{—}098972). Accordingly, utilities of VGAM49 include diagnosis, prevention and treatment of diseases and clinical conditions associated with LOC158629. LOC158943 (Accession XM_{—}018400) is another VGAM49 host target gene. LOC158943 BINDING SITE is HOST TARGET binding site found in the 3′ untranslated region of mRNA encoded by LOC158943, corresponding to a HOST TARGET binding site such as BINDING SITE I, BINDING SITE II or BINDING SITE III. Table 2 illustrates the complementarity of the nucleotide sequences of LOC158943 BINDING SITE, designated SEQ ID:2572, to the nucleotide sequence of VGAM49 RNA, herein designated VGAM RNA, also designated SEQ ID:384.

Another function of VGAM49 is therefore inhibition of LOC158943 (Accession XM_{—}018400). Accordingly, utilities of VGAM49 include diagnosis, prevention and treatment of diseases and clinical conditions associated with LOC158943. LOC170082 (Accession XM_{—}093092) is another VGAM49 host target gene. LOC170082 BINDING SITE is HOST TARGET binding site found in the 3′ untranslated region of mRNA encoded by LOC170082, corresponding to a HOST TARGET binding site such as BINDING SITE I, BINDING SITE II or BINDING SITE III. Table 2 illustrates the complementarity of the nucleotide sequences of LOC170082 BINDING SITE, designated SEQ ID:3254, to the nucleotide sequence of VGAM49 RNA, herein designated VGAM RNA, also designated SEQ ID:384.

Another function of VGAM49 is therefore inhibition of LOC170082 (Accession XM_{—}093092). Accordingly, utilities of VGAM49 include diagnosis, prevention and treatment of diseases and clinical conditions associated with LOC170082. LOC199920 (Accession XM_{—}114056) is another VGAM49 host target gene. LOC199920 BINDING SITE is HOST TARGET binding site found in the 5′ untranslated region of mRNA encoded by LOC199920, corresponding to a HOST TARGET binding site such as BINDING SITE I, BINDING SITE II or BINDING SITE III. Table 2 illustrates the complementarity of the nucleotide sequences of LOC199920 BINDING SITE, designated SEQ ID:3436, to the nucleotide sequence of VGAM49 RNA, herein designated VGAM RNA, also designated SEQ ID:384.

Another function of VGAM49 is therefore inhibition of LOC199920 (Accession XM_{—}114056). Accordingly, utilities of VGAM49 include diagnosis, prevention and treatment of diseases and clinical conditions associated with LOC199920. LOC219667 (Accession XM_{—}166098) is another VGAM49 host target gene. LOC219667 BINDING SITE is HOST TARGET binding site found in the 3′ untranslated region of mRNA encoded by LOC219667, corresponding to a HOST TARGET binding site such as BINDING SITE I, BINDING SITE II or BINDING SITE III. Table 2 illustrates the complementarity of the nucleotide sequences of LOC219667 BINDING SITE, designated SEQ ID:3516, to the nucleotide sequence of VGAM49 RNA, herein designated VGAM RNA, also designated SEQ ID:384.

Another function of VGAM49 is therefore inhibition of LOC219667 (Accession XM_{—}166098). Accordingly, utilities of VGAM49 include diagnosis, prevention and treatment of diseases and clinical conditions associated with LOC219667. LOC220279 (Accession XM_{—}169083) is another VGAM49 host target gene. LOC220279 BINDING SITE is HOST TARGET binding site found in the 5′ untranslated region of mRNA encoded by LOC220279, corresponding to a HOST TARGET binding site such as BINDING SITE I, BINDING SITE II or BINDING SITE III. Table 2 illustrates the complementarity of the nucleotide sequences of LOC220279 BINDING SITE, designated SEQ ID:3653, to the nucleotide sequence of VGAM49 RNA, herein designated VGAM RNA, also designated SEQ ID:384.

Another function of VGAM49 is therefore inhibition of LOC220279 (Accession XM_{—}169083). Accordingly, utilities of VGAM49 include diagnosis, prevention and treatment of diseases and clinical conditions associated with LOC220279. LOC221830 (Accession XM_{—}166508) is another VGAM49 host target gene. LOC221830 BINDING SITE is HOST TARGET binding site found in the 3′ untranslated region of mRNA encoded by LOC221830, corresponding to a HOST TARGET binding site such as BINDING SITE I, BINDING SITE II or BINDING SITE III. Table 2 illustrates the complementarity of the nucleotide sequences of LOC221830 BINDING SITE, designated SEQ ID:3567, to the nucleotide sequence of VGAM49 RNA, herein designated VGAM RNA, also designated SEQ ID:384.

Another function of VGAM49 is therefore inhibition of LOC221830 (Accession XM_{—}166508). Accordingly, utilities of VGAM49 include diagnosis, prevention and treatment of diseases and clinical conditions associated with LOC221830. LOC222166 (Accession XM_{—}168425) is another VGAM49 host target gene. LOC222166 BINDING SITE is HOST TARGET binding site found in the 3′ untranslated region of mRNA encoded by LOC222166, corresponding to a HOST TARGET binding site such as BINDING SITE I, BINDING SITE II or BINDING SITE III. Table 2 illustrates the complementarity of the nucleotide sequences of LOC222166 BINDING SITE, designated SEQ ID:3635, to the nucleotide sequence of VGAM49 RNA, herein designated VGAM RNA, also designated SEQ ID:384.

Another function of VGAM49 is therefore inhibition of LOC222166 (Accession XM_{—}168425). Accordingly, utilities of VGAM49 include diagnosis, prevention and treatment of diseases and clinical conditions associated with LOC222166. LOC254440 (Accession XM_{—}173126) is another VGAM49 host target gene. LOC254440 BINDING SITE is HOST TARGET binding site found in the 3′ untranslated region of mRNA encoded by LOC254440, corresponding to a HOST TARGET binding site such as BINDING SITE I, BINDING SITE II or BINDING SITE III. Table 2 illustrates the complementarity of the nucleotide sequences of LOC254440 BINDING SITE, designated SEQ ID:3729, to the nucleotide sequence of VGAM49 RNA, herein designated VGAM RNA, also designated SEQ ID:384.

Another function of VGAM49 is therefore inhibition of LOC254440 (Accession XM_{—}173126). Accordingly, utilities of VGAM49 include diagnosis, prevention and treatment of diseases and clinical conditions associated with LOC254440. LOC51123 (Accession XM_{—}018277) is another VGAM49 host target gene. LOC51123 BINDING SITE is HOST TARGET binding site found in the 3′ untranslated region of mRNA encoded by LOC51123, corresponding to a HOST TARGET binding site such as BINDING SITE I, BINDING SITE II or BINDING SITE III. Table 2 illustrates the complementarity of the nucleotide sequences of LOC51123 BINDING SITE, designated SEQ ID:2571, to the nucleotide sequence of VGAM49 RNA, herein designated VGAM RNA, also designated SEQ ID:384.

Another function of VGAM49 is therefore inhibition of LOC51123 (Accession XM_{—}018277). Accordingly, utilities of VGAM49 include diagnosis, prevention and treatment of diseases and clinical conditions associated with LOC51123. LOC51596 (Accession NM_{—}015921) is another VGAM49 host target gene. LOC51596 BINDING SITE is HOST TARGET binding site found in the 5′ untranslated region of mRNA encoded by LOC51596, corresponding to a HOST TARGET binding site such as BINDING SITE I, BINDING SITE II or BINDING SITE III. Table 2 illustrates the complementarity of the nucleotide sequences of LOC51596 BINDING SITE, designated SEQ ID:1651, to the nucleotide sequence of VGAM49 RNA, herein designated VGAM RNA, also designated SEQ ID:384.

Another function of VGAM49 is therefore inhibition of LOC51596 (Accession NM_{—}015921). Accordingly, utilities of VGAM49 include diagnosis, prevention and treatment of diseases and clinical conditions associated with LOC51596. LOC84570 (Accession NM_{—}032518) is another VGAM49 host target gene. LOC84570 BINDING SITE is HOST TARGET binding site found in the 5′ untranslated region of mRNA encoded by LOC84570, corresponding to a HOST TARGET binding site such as BINDING SITE I, BINDING SITE II or BINDING SITE III. Table 2 illustrates the complementarity of the nucleotide sequences of LOC84570 BINDING SITE, designated SEQ ID:2257, to the nucleotide sequence of VGAM49 RNA, herein designated VGAM RNA, also designated SEQ ID:384.

Another function of VGAM49 is therefore inhibition of LOC84570 (Accession NM_{—}032518). Accordingly, utilities of VGAM49 include diagnosis, prevention and treatment of diseases and clinical conditions associated with LOC84570. LOC92710 (Accession XM_{—}046811) is another VGAM49 host target gene. LOC92710 BINDING SITE is HOST TARGET binding site found in the 5′ untranslated region of mRNA encoded by LOC92710, corresponding to a HOST TARGET binding site such as BINDING SITE I, BINDING SITE II or BINDING SITE III. Table 2 illustrates the complementarity of the nucleotide sequences of LOC92710 BINDING SITE, designated SEQ ID:2885, to the nucleotide sequence of VGAM49 RNA, herein designated VGAM RNA, also designated SEQ ID:384.

Another function of VGAM49 is therefore inhibition of LOC92710 (Accession XM_{—}046811). Accordingly, utilities of VGAM49 include diagnosis, prevention and treatment of diseases and clinical conditions associated with LOC92710. FIG. 1 further provides a conceptual description of a novel bioinformatically detected viral gene of the present invention, referred to here as Viral Genomic Address Messenger 50 (VGAM50) viral gene, which modulates expression of respective host target genes thereof, the function and utility of which host target genes is known in the art.

VGAM50 is a novel bioinformatically detected regulatory, non protein coding, viral micro RNA (miRNA) gene. The method by which VGAM50 was detected is described hereinabove with reference to FIGS. 1-8.

VGAM50 gene, herein designated VGAM GENE, is a viral gene contained in the genome of Vaccinia Virus. VGAM50 host target gene, herein designated VGAM HOST TARGET GENE, is a human gene contained in the human genome.

VGAM50 gene encodes a VGAM50 precursor RNA, herein designated VGAM PRECURSOR RNA. Similar to other miRNA genes, and unlike most ordinary genes, VGAM50 precursor RNA does not encode a protein. A nucleotide sequence identical or highly similar to the nucleotide sequence of VGAM50 precursor RNA is designated SEQ ID:36, and is provided hereinbelow with reference to the sequence listing part. Nucleotide sequence SEQ ID:36 is located at position 2870 relative to the genome of Vaccinia Virus.

VGAM50 precursor RNA folds onto itself, forming VGAM50 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM50 folded precursor RNA into VGAM50 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM50 RNA is designated SEQ ID:385, and is provided hereinbelow with reference to the sequence listing part.

VGAM50 host target gene, herein designated VGAM HOST TARGET GENE, encodes a corresponding messenger RNA, VGAM50 host target RNA, herein designated VGAM HOST TARGET RNA. VGAM50 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM50 RNA, herein designated VGAM RNA, binds complementarily to one or more host target binding sites located in untranslated regions of VGAM50 host target RNA, herein designated VGAM HOST TARGET RNA. This complementary binding is due to the fact that the nucleotide sequence of VGAM50 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM50 RNA, herein designated VGAM RNA, may have a different number of host target binding sites in untranslated regions of a VGAM50 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM50 RNA, herein designated VGAM RNA, to host target binding sites on VGAM50 host target RNA, herein designated VGAM HOST TARGET RNA, such as BINDING SITE I, BINDING SITE II and BINDING SITE III, inhibits translation of VGAM50 host target RNA into VGAM50 host target protein, herein designated VGAM HOST TARGET PROTEIN. VGAM host target protein is therefore outlined by a broken line.

It is appreciated that VGAM50 host target gene, herein designated VGAM HOST TARGET GENE, in fact represents a plurality of VGAM50 host target genes. The mRNA of each one of this plurality of VGAM50 host target genes comprises one or more host target binding sites, each having a nucleotide sequence which is at least partly complementary to VGAM50 RNA, herein designated VGAM RNA, and which when bound by VGAM50 RNA causes inhibition of translation of respective one or more VGAM50 host target proteins.

It is further appreciated by one skilled in the art that the mode of translational inhibition illustrated by FIG. 1 with specific reference to translational inhibition exerted by VGAM50 gene, herein designated VGAM GENE, on one or more VGAM50 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM50 is inhibition of expression of host target genes, as part of a novel viral mechanism of attacking a host. Accordingly, utilities of VGAM50 include diagnosis, prevention and treatment of viral infection by Vaccinia Virus. Specific functions, and accordingly utilities, of VGAM50 correlate with, and may be deduced from, the identity of the host target genes which VGAM50 binds and inhibits, and the function of these host target genes, as elaborated hereinbelow.

Nucleotide sequences of the VGAM50 precursor RNA, herein designated VGAM PRECURSOR RNA, and of the ‘diced’ VGAM50 RNA, herein designated VGAM RNA, and a schematic representation of the secondary folding of VGAM50 folded precursor RNA, herein designated VGAM FOLDED PRECURSOR RNA, of VGAM50 are further described hereinbelow with reference to Table 1.

Nucleotide sequences of host target binding sites, such as BINDING SITE-I, BINDING SITE-II and BINDING SITE-III of FIG. 1, found on VGAM50 host target RNA, and schematic representation of the complementarity of each of these host target binding sites to VGAM50 RNA, herein designated VGAM RNA, are described hereinbelow with reference to Table 2.

As mentioned hereinabove with reference to FIG. 1, a function of VGAM50 gene, herein designated VGAM is inhibition of expression of VGAM50 target genes. It is appreciated that specific functions, and accordingly utilities, of VGAM50 correlate with, and may be deduced from, the identity of the target genes which VGAM50 binds and inhibits, and the function of these target genes, as elaborated hereinbelow.

Fanconi Anemia, Complementation Group F (FANCF, Accession NM_{—}022725) is a VGAM50 host target gene. FANCF BINDING SITE is HOST TARGET binding site found in the 3′ untranslated region of mRNA encoded by FANCF, corresponding to a HOST TARGET binding site such as BINDING SITE I, BINDING SITE II or BINDING SITE III. Table 2 illustrates the complementarity of the nucleotide sequences of FANCF BINDING SITE, designated SEQ ID:1997, to the nucleotide sequence of VGAM50 RNA, herein designated VGAM RNA, also designated SEQ ID:385.

A function of VGAM50 is therefore inhibition of Fanconi Anemia, Complementation Group F (FANCF, Accession NM_{—}022725). Accordingly, utilities of VGAM50 include diagnosis, prevention and treatment of diseases and clinical conditions associated with FANCF. G Protein-coupled Receptor 65 (GPR65, Accession XM_{—}007392) is another VGAM50 host target gene. GPR65 BINDING SITE is HOST TARGET binding site found in the 5′ untranslated region of mRNA encoded by GPR65, corresponding to a HOST TARGET binding site such as BINDING SITE I, BINDING SITE II or BINDING SITE III. Table 2 illustrates the complementarity of the nucleotide sequences of GPR65 BINDING SITE, designated SEQ ID:2544, to the nucleotide sequence of VGAM50 RNA, herein designated VGAM RNA, also designated SEQ ID:385.

Another function of VGAM50 is therefore inhibition of G Protein-coupled Receptor 65 (GPR65, Accession XM_{—}007392). Accordingly, utilities of VGAM50 include diagnosis, prevention and treatment of diseases and clinical conditions associated with GPR65. Lamin B1 (LMNB1, Accession NM_{—}005573) is another VGAM50 host target gene. LMNB1 BINDING SITE is HOST TARGET binding site found in the 3′ untranslated region of mRNA encoded by LMNB1, corresponding to a HOST TARGET binding site such as BINDING SITE I, BINDING SITE II or BINDING SITE III. Table 2 illustrates the complementarity of the nucleotide sequences of LMNB1 BINDING SITE, designated SEQ ID:1226, to the nucleotide sequence of VGAM50 RNA, herein designated VGAM RNA, also designated SEQ ID:385.

Another function of VGAM50 is therefore inhibition of Lamin B1 (LMNB1, Accession NM_{—}005573), a gene which is thought to provide a framework for the nuclear envelope and may also interact with chromatin. Accordingly, utilities of VGAM50 include diagnosis, prevention and treatment of diseases and clinical conditions associated with LMNB1. The function of LMNB1 has been established by previous studies. Lamins are the major components of the nuclear lamina which underlies the nuclear envelope of eukaryotic cells (see OMIM Ref. No. lamin A, 150330). Maeno et al. (1995) noted that lamins are members of the intermediate filament protein family. Vertebrate lamins are classified into 2 types, A and B, and mammalian somatic cells show 2 species of each type: lamins A and C for the A type and B1 and B2 for the B type. In addition, germ-cell-specific lamins have been reported for both types (Furukawa and Hotta, 1993 and Furukawa et al., 1994). While A-type lamins are expressed in a developmentally controlled manner, B-type lamins are expressed in all kinds of cells. Although the role of lamins may be primarily structural, information about other functions such as interactions with interphase chromatin and involvement in DNA replication is accumulating The lamin B gene in the mouse is located on chromosome 18 (Justice et al., 1992) in a region of linkage homology to the long arm of human chromosome 5. This gene (Lmnb1) was isolated by Maeno et al. (1995). Structural analysis showed that the lamin B1 gene spans about 43 kb of the genome and consists of 11 exons and 10 introns. Exon/intron structure of the B1 gene clearly showed the conserved organization among the intermediate filament protein family genes. The presumptive promoter region has high GC content and contains a CAAT box and multiple Sp1 sites but no classical TATA box, suggesting to the authors that the lamin B1 gene has a typical housekeeping gene promoter with a CpG island

Full details of the abovementioned studies are described in the following publications, the disclosure of which are hereby incorporated by reference:
Maeno, H.; Sugimoto, K.; Nakajima, N.: Genomic structure of the mouse gene (Lmnb1) encoding nuclear lamin B1. Genomics 30: 342-346, 1995.; and
Maeno, H.; Sugimoto, K.; Nakajima, N.: Genomic structure of the mouse gene (Lmnb1) encoding nuclear lamin B1. Genomics 30: 342-346, 1995.

Further studies establishing the function and utilities of LMNB1 are found in John Hopkins OMIM database record ID 150340, and in sited publications numbered 3012-3013, 2790, 3006-300 and 3011 listed in the bibliography section hereinbelow, which are also hereby incorporated by reference. Multiple Endocrine Neoplasia I (MEN1, Accession XM_{—}167804) is another VGAM50 host target gene. MEN1 BINDING SITE is HOST TARGET binding site found in the 5′ untranslated region of mRNA encoded by MEN1, corresponding to a HOST TARGET binding site such as BINDING SITE I, BINDING SITE II or BINDING SITE III. Table 2 illustrates the complementarity of the nucleotide sequences of MEN1 BINDING SITE, designated SEQ ID:3600, to the nucleotide sequence of VGAM50 RNA, herein designated VGAM RNA, also designated SEQ ID:385.

Another function of VGAM50 is therefore inhibition of Multiple Endocrine Neoplasia I (MEN1, Accession XM_{—}167804). Accordingly, utilities of VGAM50 include diagnosis, prevention and treatment of diseases and clinical conditions associated with MEN1. Pleiomorphic Adenoma Gene 1 (PLAG1, Accession NM_{—}002655) is another VGAM50 host target gene. PLAG1 BINDING SITE is HOST TARGET binding site found in the 3′ untranslated region of mRNA encoded by PLAG1, corresponding to a HOST TARGET binding site such as BINDING SITE I, BINDING SITE II or BINDING SITE III. Table 2 illustrates the complementarity of the nucleotide sequences of PLAG1 BINDING SITE, designated SEQ ID:944, to the nucleotide sequence of VGAM50 RNA, herein designated VGAM RNA, also designated SEQ ID:385.

Another function of VGAM50 is therefore inhibition of Pleiomorphic Adenoma Gene 1 (PLAG1, Accession NM_{—}002655), a gene which contains a zinc finger domain. Accordingly, utilities of VGAM50 include diagnosis, prevention and treatment of diseases and clinical conditions associated with PLAG1. The function of PLAG1 has been established by previous studies. Pleomorphic adenomas are benign epithelial tumors originating from the major and minor salivary glands (see OMIM Ref. No. 181030). They are characterized by recurrent chromosome translocations; the most common abnormalities involve chromosome 8, with consistent breakpoints at band q12. Kas et al. (1997) described the construction of 2 nonoverlapping YAC contigs covering about 75% of human chromosome band 8q12, which spans approximately 9 Mb of genomic DNA and includes a number of known genes such as MOS (OMIM Ref. No. 190060) and LYN (OMIM Ref. No. 165120), as well as novel genes and expressed sequence tags (ESTs). By fluorescence in situ hybridization, the authors determined that the majority of pleomorphic adenoma 8q12 breakpoints clustered within a 2-Mb contig that was mapped to the centromeric region of 8q12 and that was covered by 34 overlapping YAC clones, and tagged by 31 markers with an average spacing of 65 kb. Nine of 11 primary adenomas with 8q12 abnormalities had breakpoints mapping within a 300-kb interval. By searching sequence databases with sequence tagged sites (STSs) located within the 300-kb region, Kas et al. (1997) identified an EST with sequence identity to one of the STSs. Northern blot analysis using this EST detected a 7.5-kb transcript representing pleomorphic adenoma gene-1 (PLAG1). The authors cloned human fetal kidney PLAG1 cDNAs and found that the PLAG1 gene contains 5 exons. Southern blot analysis of DNA from pleomorphic adenomas with t(3;8) detected rearrangements in the 5-prime noncoding region of the PLAG1 gene. Using 5-prime RACE or RT-PCR, the authors generated hybrid transcripts consisting of PLAG1 and beta-1-catenin (CTNNB1; 116806) from every primary tumor analyzed. Northern blot analysis of 3 pleomorphic adenomas with t(3;8) and 1 adenoma with a variant t(8;15) revealed that PLAG1 expression was activated by the translocations in all 4 tumors. Kas et al. (1997) detected the 7.5-kb PLAG1 transcript in normal human fetal lung, fetal liver, and fetal kidney, but not in the corresponding adult tissues, adult salivary gland, or fetal brain; CTNNB1 appeared to be ubiquitously expressed. The deduced PLAG1 protein has 2 potential nuclear localization signals in the N-terminal region, 7 zinc finger domains, and a serine-rich C terminus. Astrom et al. (1999) found overexpression of PLAG1 in 23 of 47 primary benign and malignant pleomorphic adenomas of the salivary glands. In 5 adenomas with a normal karyotype, fusion transcripts were found in 3; PLAG1 and CTNNB1 were fused in 1 case, and in 2 others PLAG1 was fused with the gene encoding transcription elongation factor SII (OMIM Ref. No. 601425). The fusions occurred in the 5-prime noncoding region of PLAG1, leading to exchange of regulatory control elements and, as a consequence, activation of PLAG1 gene expression. Because all of the cases had grossly normal karyotypes, the rearrangements must result from cryptic rearrangements.

Full details of the abovementioned studies are described in the following publications, the disclosure of which are hereby incorporated by reference:
Astrom, A.-K.; Voz, M. L.; Kas, K.; Roijer, E.; Wedell, B.; Mandahl, N.; Van de Ven, W.; Mark, J.; Stenman, G.: Conserved mechanism of PLAG1 activation in salivary gland tumors with and without chromosome 8q12 abnormalities: identification of SII as a new fusion partner gene. Cancer Res. 59: 918-923, 1999.; and
Kas, K.; Roijer, E.; Voz, M.; Meyen, E.; Stenman, G.; Van de Ven, W. J. M.: A 2-Mb YAC contig and physical map covering the chromosome 8q12 breakpoint cluster region in pleomorphic ad.

Further studies establishing the function and utilities of PLAG are found in John Hopkins OMIM database record ID 603026, and in sited publications numbered 124 and 1338-1339 listed in the bibliography section hereinbelow, which are also hereby incorporated by reference. S100 Calcium Binding Protein All (calgizzarin) (S100A11, Accession NM_{—}005620) is another VGAM50 host target gene. S100A11 BINDING SITE is HOST TARGET binding site found in the 3′ untranslated region of mRNA encoded by S100A11, corresponding to a HOST TARGET binding site such as BINDING SITE I, BINDING SITE II or BINDING SITE III. Table 2 illustrates the complementarity of the nucleotide sequences of S100A11 BINDING SITE, designated SEQ ID:1231, to the nucleotide sequence of VGAM50 RNA, herein designated VGAM RNA, also designated SEQ ID:385.

Another function of VGAM50 is therefore inhibition of S100 Calcium Binding Protein All (calgizzarin) (S100A11, Accession NM_{—}005620), a gene which is a tissue/cell type specific calcium-binding protein that interacts with target proteins to link extracellular stimuli to cellular responses; member of the S100 family. Accordingly, utilities of VGAM50 include diagnosis, prevention and treatment of diseases and clinical conditions associated with S100A11. The function of S100A11 has been established by previous studies. Proteins of the S100 protein family belong to the large group of EF-hand calcium-binding proteins. Todoroki et al. (1991) purified an S100 protein from chicken gizzard that they called calgizzarin. Watanabe et al. (1991) isolated a cDNA encoding rabbit calgizzarin. Tanaka et al. (1995) identified and sequenced a cDNA encoding a human calgizzarin homolog. They found that the expression of human calgizzarin was remarkably elevated in colorectal cancers compared with that in normal colorectal mucosa. Tomasetto et al. (1995) reported that calgizzarin, or MLN70, is one of several genes expressed in breast cancer-derived metastatic axillary lymph nodes but not in normal lymph nodes or breast fibroadenomas. By in situ hybridization, Moog-Lutz et al. (1995) mapped the calgizzarin, or S100C, gene to 1q21. By analysis of clones from this region, Wicki et al. (1996) determined that S100A11 is part of the S100 gene cluster and is located near S100A10 (OMIM Ref. No. 114085).

Full details of the abovementioned studies are described in the following publications, the disclosure of which are hereby incorporated by reference:
Watanabe, M.; Ando, Y.; Todoroki, H.; Minami, H.; Hidaka, H.: Molecular cloning and sequencing of a cDNA clone encoding a new calcium binding protein, named calgizzarin, from rabbit lung. Biochem. Biophys. Res. Commun. 181: 644-649, 1991.; and
Wicki, R.; Marenholz, I.; Mischke, D.; Schafer, B. W.; Heizmann, C. W.: Characterization of the human S100A12 (calgranulin C, p6, CAAF1, CGRP) gene, a new member of the S100 gene lust.

Further studies establishing the function and utilities of S100A11 are found in John Hopkins OMIM database record ID 603114, and in sited publications numbered 299-301, 135 and 1938 listed in the bibliography section hereinbelow, which are also hereby incorporated by reference. Solute Carrier Family 9 (sodium/hydrogen exchanger), Isoform 6 (SLC9A6, Accession NM_{—}006359) is another VGAM50 host target gene. SLC9A6 BINDING SITE is HOST TARGET binding site found in the 3′ untranslated region of mRNA encoded by SLC9A6, corresponding to a HOST TARGET binding site such as BINDING SITE I, BINDING SITE II or BINDING SITE III. Table 2 illustrates the complementarity of the nucleotide sequences of SLC9A6 BINDING SITE, designated SEQ ID:1290, to the nucleotide sequence of VGAM50 RNA, herein designated VGAM RNA, also designated SEQ ID:385.

Another function of VGAM50 is therefore inhibition of Solute Carrier Family 9 (sodium/hydrogen exchanger), Isoform 6 (SLC9A6, Accession NM_{—}006359), a gene which is involved electroneutral exchange of protons for na+ and k+ across the mitochondrial inner membrane. Accordingly, utilities of VGAM50 include diagnosis, prevention and treatment of diseases and clinical conditions associated with SLC9A6. The function of SLC9A6 has been established by previous studies. By searching sequence databases for proteins with sequence similarity to the S. cerevisiae mitochondrial sodium/hydrogen exchanger Nha2, Numata et al. (1998) identified the deduced protein product of the KIAA0267 cDNA (Nagase et al., 1996), SLC9A6. The KIAA0267-encoded protein shares 30% amino acid sequence identity with S. cerevisiae Nha2, and approximately 20 to 24% identity with the mammalian NHE isoforms NHE1 to NHE5 (see OMIM Ref. No. SLC9A5; 600477). Numata et al. (1998), who concluded that the KIAA0267 cDNA lacks 5-prime coding sequence, isolated a human cDNA containing the complete coding sequence of SLC9A6, which they called NHE6. The deduced 669-amino acid SLC9A6 protein has 12 putative membrane-spanning segments within the N-terminal region, and a hydrophilic C terminus, similar to the topologies predicted for other NHEs. In addition, SLC9A6 has a putative mitochondrial inner membrane targeting signal at its N terminus. Northern blot analysis detected an approximately 5.5-kb SLC9A6 transcript that was ubiquitously expressed, with the most abundant expression in mitochondrion-rich tissues such as brain, skeletal muscle, and heart. Fluorescence microscopy suggested that SLC9A6 localizes to mitochondria. Numata et al. (1998) deleted the S. cerevisiae NHA2 gene by homologous disruption and found that benzamil-inhibitable, acid-activated sodium uptake into mitochondria was abolished in the mutant strain. The mutant strain also showed retarded growth on nonfermentable carbon sources and severely reduced survival during the stationary phase of the cell cycle compared with the parental strain, consistent with a defect in aerobic metabolism. The authors suggested that Nha2 and SLC9A6 are homologous sodium/hydrogen exchangers that are important for mitochondrial function.

Full details of the abovementioned studies are described in the following publications, the disclosure of which are hereby incorporated by reference:
Nagase, T.; Seki, N.; Ishikawa, K.; Ohira, M.; Kawarabayasi, Y.; Ohara, O.; Tanaka, A.; Kotani, H.; Miyajima, N.; Nomura, N.: Prediction of the coding sequences of unidentified human genes. VI. The coding sequences of 80 new genes (KIAA0201-KIAA0280) deduced by analysis of cDNA clones from cell line KG-1 and brain. DNA Res. 3: 321-329, 1996. Note: Supplement: DNA Res. 3: 341-354, 1996.; and
Numata, M.; Petrecca, K.; Lake, N.; Orlowski, J.: Identification of a mitochondrial Na+/H+ exchanger. J. Biol. Chem. 273: 6951-6959, 1998.

Further studies establishing the function and utilities of SLC9A6 are found in John Hopkins OMIM database record ID 300231, and in sited publications numbered 2015-2016 listed in the bibliography section hereinbelow, which are also hereby incorporated by reference. Superoxide Dismutase 2, Mitochondrial (SOD2, Accession NM_{—}000636) is another VGAM50 host target gene. SOD2 BINDING SITE is HOST TARGET binding site found in the 3′ untranslated region of mRNA encoded by SOD2, corresponding to a HOST TARGET binding site such as BINDING SITE I, BINDING SITE II or BINDING SITE III. Table 2 illustrates the complementarity of the nucleotide sequences of SOD2 BINDING SITE, designated SEQ ID:766, to the nucleotide sequence of VGAM50 RNA, herein designated VGAM RNA, also designated SEQ ID:385.

Another function of VGAM50 is therefore inhibition of Superoxide Dismutase 2, Mitochondrial (SOD2, Accession NM_{—}000636), a gene which is intramitochondrial free radical scavenging enzyme. When he has polymorphism in signal peptides he could cause diseases of distribution. Accordingly, utilities of VGAM50 include diagnosis, prevention and treatment of diseases and clinical conditions associated with SOD2. The function of SOD2 has been established by previous studies. Rosenblum et al. (1996) emphasized the importance of polymorphism in signal peptides that target organelle-specific proteins to their subcellular sites of action. They discovered a polymorphism when they cloned the SOD2 gene from cell lines of normal individuals and patients with genetic diseases of premature aging. The polymorphism consisted of a single nucleotide change in the region of the DNA that encodes the signal sequence such that either an alanine or a valine was present (147460.0001). Rosenblum et al. (1996) suggested that such signal sequence polymorphism could result in diseases of distribution, where essential proteins are not properly targeted, thereby leading to absolute or relative deficiencies of critical enzymes within specific cellular compartments. They suggested that progeria (OMIM Ref. No. 176670) and related syndromes may be diseases of distribution. Hiroi et al. (1999) found an increased frequency for the SOD2-VV genotype (homozygosity for the valine (V) allele vs the alanine allele) in Japanese with nonfamilial idiopathic cardiomyopathy (IDC) and suggested that this polymorphism may collaborate with the DRP1*1401 allele of the HLA-DRB1 gene (OMIM Ref. No. 142857) in controlling the susceptibility to nonfamilial IDC

Full details of the abovementioned studies are described in the following publications, the disclosure of which are hereby incorporated by reference:
Hiroi, S.; Harada, H.; Nishi, H.; Satoh, M.; Nagai, R.; Kimura, A.: Polymorphisms in the SOD2 and HLA-DRB1 genes are associated with nonfamilial idiopathic dilated cardiomyopathy in Japanese. Biochem. Biophys. Res. Commun. 261: 332-339, 1999.; and
Rosenblum, J. S.; Gilula, N. B.; Lerner, R. A.: On signal sequence polymorphisms and diseases of distribution. Proc. Nat. Acad. Sci. 93: 4471-4473, 1996.

Further studies establishing the function and utilities of SOD2 are found in John Hopkins OMIM database record ID 147460, and in sited publications numbered 798-802, 866-80 and 867-809 listed in the bibliography section hereinbelow, which are also hereby incorporated by reference. Baculoviral IAP Repeat-containing 2 (BIRC2, Accession XM_{—}040717) is another VGAM50 host target gene. BIRC2 BINDING SITE is HOST TARGET binding site found in the 5′ untranslated region of mRNA encoded by BIRC2, corresponding to a HOST TARGET binding site such as BINDING SITE I, BINDING SITE II or BINDING SITE III. Table 2 illustrates the complementarity of the nucleotide sequences of BIRC2 BINDING SITE, designated SEQ ID:2784, to the nucleotide sequence of VGAM50 RNA, herein designated VGAM RNA, also designated SEQ ID:385.

Another function of VGAM50 is therefore inhibition of Baculoviral IAP Repeat-containing 2 (BIRC2, Accession XM_{—}040717). Accordingly, utilities of VGAM50 include diagnosis, prevention and treatment of diseases and clinical conditions associated with BIRC2. Chromosome X Open Reading Frame 1 (CXorf1, Accession NM_{—}004709) is another VGAM50 host target gene. CXorf1 BINDING SITE is HOST TARGET binding site found in the 5′ untranslated region of mRNA encoded by CXorf1, corresponding to a HOST TARGET binding site such as BINDING SITE I, BINDING SITE II or BINDING SITE III. Table 2 illustrates the complementarity of the nucleotide sequences of CXorf1 BINDING SITE, designated SEQ ID:1145, to the nucleotide sequence of VGAM50 RNA, herein designated VGAM RNA, also designated SEQ ID:385.

Another function of VGAM50 is therefore inhibition of Chromosome X Open Reading Frame 1 (CXorf1, Accession NM_{—}004709). Accordingly, utilities of VGAM50 include diagnosis, prevention and treatment of diseases and clinical conditions associated with CXorf1. KIAA0367 (Accession XM_{—}041018) is another VGAM50 host target gene. KIAA0367 BINDING SITE is HOST TARGET binding site found in the 3′ untranslated region of mRNA encoded by KIAA0367, corresponding to a HOST TARGET binding site such as BINDING SITE I, BINDING SITE II or BINDING SITE III. Table 2 illustrates the complementarity of the nucleotide sequences of KIAA0367 BINDING SITE, designated SEQ ID:2787, to the nucleotide sequence of VGAM50 RNA, herein designated VGAM RNA, also designated SEQ ID:385.

Another function of VGAM50 is therefore inhibition of KIAA0367 (Accession XM_{—}041018). Accordingly, utilities of VGAM50 include diagnosis, prevention and treatment of diseases and clinical conditions associated with KIAA0367. KIAA1321 (Accession XM_{—}030856) is another VGAM50 host target gene. KIAA1321 BINDING SITE is HOST TARGET binding site found in the 3′ untranslated region of mRNA encoded by KIAA1321, corresponding to a HOST TARGET binding site such as BINDING SITE I, BINDING SITE II or BINDING SITE III. Table 2 illustrates the complementarity of the nucleotide sequences of KIAA1321 BINDING SITE, designated SEQ ID:2627, to the nucleotide sequence of VGAM50 RNA, herein designated VGAM RNA, also designated SEQ ID:385.

Another function of VGAM50 is therefore inhibition of KIAA1321 (Accession XM_{—}030856). Accordingly, utilities of VGAM50 include diagnosis, prevention and treatment of diseases and clinical conditions associated with KIAA1321. KIAA1718 (Accession XM_{—}034823) is another VGAM50 host target gene. KIAA1718 BINDING SITE is HOST TARGET binding site found in the 3′ untranslated region of mRNA encoded by KIAA1718, corresponding to a HOST TARGET binding site such as BINDING SITE I, BINDING SITE II or BINDING SITE III. Table 2 illustrates the complementarity of the nucleotide sequences of KIAA1718 BINDING SITE, designated SEQ ID:2686, to the nucleotide sequence of VGAM50 RNA, herein designated VGAM RNA, also designated SEQ ID:385.

Another function of VGAM50 is therefore inhibition of KIAA1718 (Accession XM_{—}034823). Accordingly, utilities of VGAM50 include diagnosis, prevention and treatment of diseases and clinical conditions associated with KIAA1718. KIAA1789 (Accession XM_{—}040486) is another VGAM50 host target gene. KIAA1789 BINDING SITE is HOST TARGET binding site found in the 5′ untranslated region of mRNA encoded by KIAA1789, corresponding to a HOST TARGET binding site such as BINDING SITE I, BINDING SITE II or BINDING SITE III. Table 2 illustrates the complementarity of the nucleotide sequences of KIAA1789 BINDING SITE, designated SEQ ID:2781, to the nucleotide sequence of VGAM50 RNA, herein designated VGAM RNA, also designated SEQ ID:385.

Another function of VGAM50 is therefore inhibition of KIAA1789 (Accession XM_{—}040486). Accordingly, utilities of VGAM50 include diagnosis, prevention and treatment of diseases and clinical conditions associated with KIAA1789. PRO2964 (Accession NM_{—}018547) is another VGAM50 host target gene. PRO2964 BINDING SITE is HOST TARGET binding site found in the 3′ untranslated region of mRNA encoded by PRO2964, corresponding to a HOST TARGET binding site such as BINDING SITE I, BINDING SITE II or BINDING SITE III. Table 2 illustrates the complementarity of the nucleotide sequences of PRO2964 BINDING SITE, designated SEQ ID:1842, to the nucleotide sequence of VGAM50 RNA, herein designated VGAM RNA, also designated SEQ ID:385.

Another function of VGAM50 is therefore inhibition of PRO2964 (Accession NM_{—}018547). Accordingly, utilities of VGAM50 include diagnosis, prevention and treatment of diseases and clinical conditions associated with PRO2964. Solute Carrier Family 17 (sodium-dependent inorganic phosphate cotransporter), Member 6 (SLC17A6, Accession NM_{—}020346) is another VGAM50 host target gene. SLC17A6 BINDING SITE is HOST TARGET binding site found in the 3′ untranslated region of mRNA encoded by SLC17A6, corresponding to a HOST TARGET binding site such as BINDING SITE I, BINDING SITE II or BINDING SITE III. Table 2 illustrates the complementarity of the nucleotide sequences of SLC17A6 BINDING SITE, designated SEQ ID:1912, to the nucleotide sequence of VGAM50 RNA, herein designated VGAM RNA, also designated SEQ ID:385.

Another function of VGAM50 is therefore inhibition of Solute Carrier Family 17 (sodium-dependent inorganic phosphate cotransporter), Member 6 (SLC17A6, Accession NM_{—}020346). Accordingly, utilities of VGAM50 include diagnosis, prevention and treatment of diseases and clinical conditions associated with SLC17A6. TNF Receptor-associated Factor 3 (TRAF3, Accession XM_{—}007256) is another VGAM50 host target gene. TRAF3 BINDING SITE is HOST TARGET binding site found in the 5′ untranslated region of mRNA encoded by TRAF3, corresponding to a HOST TARGET binding site such as BINDING SITE I, BINDING SITE II or BINDING SITE III. Table 2 illustrates the complementarity of the nucleotide sequences of TRAF3 BINDING SITE, designated SEQ ID:2542, to the nucleotide sequence of VGAM50 RNA, herein designated VGAM RNA, also designated SEQ ID:385.

Another function of VGAM50 is therefore inhibition of TNF Receptor-associated Factor 3 (TRAF3, Accession XM_{—}007256). Accordingly, utilities of VGAM50 include diagnosis, prevention and treatment of diseases and clinical conditions associated with TRAF3. LOC151361 (Accession XM_{—}098048) is another VGAM50 host target gene. LOC151361 BINDING SITE is HOST TARGET binding site found in the 3′ untranslated region of mRNA encoded by LOC151361, corresponding to a HOST TARGET binding site such as BINDING SITE I, BINDING SITE II or BINDING SITE III. Table 2 illustrates the complementarity of the nucleotide sequences of LOC151361 BINDING SITE, designated SEQ ID:3344, to the nucleotide sequence of VGAM50 RNA, herein designated VGAM RNA, also designated SEQ ID:385.

Another function of VGAM50 is therefore inhibition of LOC151361 (Accession XM_{—}098048). Accordingly, utilities of VGAM50 include diagnosis, prevention and treatment of diseases and clinical conditions associated with LOC151361. LOC158714 (Accession XM_{—}088650) is another VGAM50 host target gene. LOC158714 BINDING SITE is HOST TARGET binding site found in the 3′ untranslated region of mRNA encoded by LOC158714, corresponding to a HOST TARGET binding site such as BINDING SITE I, BINDING SITE II or BINDING SITE III. Table 2 illustrates the complementarity of the nucleotide sequences of LOC158714 BINDING SITE, designated SEQ ID:3224, to the nucleotide sequence of VGAM50 RNA, herein designated VGAM RNA, also designated SEQ ID:385.

Another function of VGAM50 is therefore inhibition of LOC158714 (Accession XM_{—}088650). Accordingly, utilities of VGAM50 include diagnosis, prevention and treatment of diseases and clinical conditions associated with LOC158714. LOC219667 (Accession XM_{—}166098) is another VGAM50 host target gene. LOC219667 BINDING SITE is HOST TARGET binding site found in the 3′ untranslated region of mRNA encoded by LOC219667, corresponding to a HOST TARGET binding site such as BINDING SITE I, BINDING SITE II or BINDING SITE III. Table 2 illustrates the complementarity of the nucleotide sequences of LOC219667 BINDING SITE, designated SEQ ID:3518, to the nucleotide sequence of VGAM50 RNA, herein designated VGAM RNA, also designated SEQ ID:385.

Another function of VGAM50 is therefore inhibition of LOC219667 (Accession XM_{—}166098). Accordingly, utilities of VGAM50 include diagnosis, prevention and treatment of diseases and clinical conditions associated with LOC219667. LOC91565 (Accession XM_{—}039231) is another VGAM50 host target gene. LOC91565 BINDING SITE is HOST TARGET binding site found in the 3′ untranslated region of mRNA encoded by LOC91565, corresponding to a HOST TARGET binding site such as BINDING SITE I, BINDING SITE II or BINDING SITE III. Table 2 illustrates the complementarity of the nucleotide sequences of LOC91565 BINDING SITE, designated SEQ ID:2749, to the nucleotide sequence of VGAM50 RNA, herein designated VGAM RNA, also designated SEQ ID:385.

Another function of VGAM50 is therefore inhibition of LOC91565 (Accession XM_{—}039231). Accordingly, utilities of VGAM50 include diagnosis, prevention and treatment of diseases and clinical conditions associated with LOC91565. FIG. 1 further provides a conceptual description of a novel bioinformatically detected viral gene of the present invention, referred to here as Viral Genomic Address Messenger 51 (VGAM51) viral gene, which modulates expression of respective host target genes thereof, the function and utility of which host target genes is known in the art.

VGAM51 is a novel bioinformatically detected regulatory, non protein coding, viral micro RNA (miRNA) gene. The method by which VGAM51 was detected is described hereinabove with reference to FIGS. 1-8.

VGAM51 gene, herein designated VGAM GENE, is a viral gene contained in the genome of Vaccinia Virus. VGAM51 host target gene, herein designated VGAM HOST TARGET GENE, is a human gene contained in the human genome.

VGAM51 gene encodes a VGAM51 precursor RNA, herein designated VGAM PRECURSOR RNA. Similar to other miRNA genes, and unlike most ordinary genes, VGAM51 precursor RNA does not encode a protein. A nucleotide sequence identical or highly similar to the nucleotide sequence of VGAM51 precursor RNA is designated SEQ ID:37, and is provided hereinbelow with reference to the sequence listing part. Nucleotide sequence SEQ ID:37 is located at position 6123 relative to the genome of Vaccinia Virus.

VGAM51 precursor RNA folds onto itself, forming VGAM51 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM51 folded precursor RNA into VGAM51 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM51 RNA is designated SEQ ID:386, and is provided hereinbelow with reference to the sequence listing part.

VGAM51 host target gene, herein designated VGAM HOST TARGET GENE, encodes a corresponding messenger RNA, VGAM51 host target RNA, herein designated VGAM HOST TARGET RNA. VGAM51 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM51 RNA, herein designated VGAM RNA, binds complementarily to one or more host target binding sites located in untranslated regions of VGAM51 host target RNA, herein designated VGAM HOST TARGET RNA. This complementary binding is due to the fact that the nucleotide sequence of VGAM51 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM51 RNA, herein designated VGAM RNA, may have a different number of host target binding sites in untranslated regions of a VGAM51 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM51 RNA, herein designated VGAM RNA, to host target binding sites on VGAM51 host target RNA, herein designated VGAM HOST TARGET RNA, such as BINDING SITE I, BINDING SITE II and BINDING SITE III, inhibits translation of VGAM51 host target RNA into VGAM51 host target protein, herein designated VGAM HOST TARGET PROTEIN. VGAM host target protein is therefore outlined by a broken line.

It is appreciated that VGAM51 host target gene, herein designated VGAM HOST TARGET GENE, in fact represents a plurality of VGAM51 host target genes. The mRNA of each one of this plurality of VGAM51 host target genes comprises one or more host target binding sites, each having a nucleotide sequence which is at least partly complementary to VGAM51 RNA, herein designated VGAM RNA, and which when bound by VGAM51 RNA causes inhibition of translation of respective one or more VGAM51 host target proteins.

It is further appreciated by one skilled in the art that the mode of translational inhibition illustrated by FIG. 1 with specific reference to translational inhibition exerted by VGAM51 gene, herein designated VGAM GENE, on one or more VGAM51 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM51 is inhibition of expression of host target genes, as part of a novel viral mechanism of attacking a host. Accordingly, utilities of VGAM51 include diagnosis, prevention and treatment of viral infection by Vaccinia Virus. Specific functions, and accordingly utilities, of VGAM51 correlate with, and may be deduced from, the identity of the host target genes which VGAM51 binds and inhibits, and the function of these host target genes, as elaborated hereinbelow.

Nucleotide sequences of the VGAM51 precursor RNA, herein designated VGAM PRECURSOR RNA, and of the ‘diced’ VGAM51 RNA, herein designated VGAM RNA, and a schematic representation of the secondary folding of VGAM51 folded precursor RNA, herein designated VGAM FOLDED PRECURSOR RNA, of VGAM51 are further described hereinbelow with reference to Table 1.

Nucleotide sequences of host target binding sites, such as BINDING SITE-I, BINDING SITE-II and BINDING SITE-III of FIG. 1, found on VGAM51 host target RNA, and schematic representation of the complementarity of each of these host target binding sites to VGAM51 RNA, herein designated VGAM RNA, are described hereinbelow with reference to Table 2.

As mentioned hereinabove with reference to FIG. 1, a function of VGAM51 gene, herein designated VGAM is inhibition of expression of VGAM51 target genes. It is appreciated that specific functions, and accordingly utilities, of VGAM51 correlate with, and may be deduced from, the identity of the target genes which VGAM51 binds and inhibits, and the function of these target genes, as elaborated hereinbelow.

Chromosome 1 Open Reading Frame 1 (Clorf1, Accession NM_{—}001213) is a VGAM51 host target gene. Clorf1 BINDING SITE is HOST TARGET binding site found in the 5′ untranslated region of mRNA encoded by Clorf1, corresponding to a HOST TARGET binding site such as BINDING SITE I, BINDING SITE II or BINDING SITE III. Table 2 illustrates the complementarity of the nucleotide sequences of Clorf1 BINDING SITE, designated SEQ ID:809, to the nucleotide sequence of VGAM51 RNA, herein designated VGAM RNA, also designated SEQ ID:386.

A function of VGAM51 is therefore inhibition of Chromosome 1 Open Reading Frame 1 (Clorf1, Accession NM_{—}001213). Accordingly, utilities of VGAM51 include diagnosis, prevention and treatment of diseases and clinical conditions associated with Clorf1. Lecithin Retinol Acyltransferase (phosphatidyIcholine—retinol O-acyltransferase) (LRAT, Accession XM_{—}011181) is another VGAM51 host target gene. LRAT BINDING SITE is HOST TARGET binding site found in the 3′ untranslated region of mRNA encoded by LRAT, corresponding to a HOST TARGET binding site such as BINDING SITE I, BINDING SITE II or BINDING SITE III. Table 2 illustrates the complementarity of the nucleotide sequences of LRAT BINDING SITE, designated SEQ ID:2556, to the nucleotide sequence of VGAM51 RNA, herein designated VGAM RNA, also designated SEQ ID:386.

Another function of VGAM51 is therefore inhibition of Lecithin Retinol Acyltransferase (phosphatidyIcholine—retinol O-acyltransferase) (LRAT, Accession XM_{—}011181). Accordingly, utilities of VGAM51 include diagnosis, prevention and treatment of diseases and clinical conditions associated with LRAT. V-myb Myeloblastosis Viral Oncogene Homolog (avian)-like 1 (MYBL1, Accession XM_{—}034274) is another VGAM51 host target gene. MYBL1 BINDING SITE is HOST TARGET binding site found in the 3′ untranslated region of mRNA encoded by MYBL1, corresponding to a HOST TARGET binding site such as BINDING SITE I, BINDING SITE II or BINDING SITE III. Table 2 illustrates the complementarity of the nucleotide sequences of MYBL1 BINDING SITE, designated SEQ ID:2679, to the nucleotide sequence of VGAM51 RNA, herein designated VGAM RNA, also designated SEQ ID:386.

Another function of VGAM51 is therefore inhibition of V-myb Myeloblastosis Viral Oncogene Homolog (avian)-like 1 (MYBL1, Accession XM_{—}034274), a gene which could have a role in the proliferation and/or differentiation of neurogenic, spermatogenic and b-lymphoid cells. Accordingly, utilities of VGAM51 include diagnosis, prevention and treatment of diseases and clinical conditions associated with MYBL1. The function of MYBL1 has been established by previous studies. Nomura et al. (1988) isolated and characterized cDNA clones for 2 human MYB-related genes, AMYB and BMYB (OMIM Ref. No. 601415). Using probes in Southern blot analysis of rodent-human hybrid DNAs, Barletta et al. (1991) localized the MYBL1 locus to 8cen-q22 and refined the localization to 8q22-q23 by in situ hybridization. Takahashi et al. (1995) found that MYBL1 mRNA is expressed mainly in testis and peripheral blood leukocytes. AMYB could activate transcription from the promoter-containing MYB-binding sites in all cells examined. In addition to the 2 domains (a DNA-binding domain and a transcriptional activation domain), 2 negative regulatory domains were identified in the MYBL1 gene. These results indicated that the gene functions as a transcriptional activator and that the regulatory mechanism of gene activity is similar to that of the MYB (OMIM Ref. No. 189990) gene.

Full details of the abovementioned studies are described in the following publications, the disclosure of which are hereby incorporated by reference:
Nomura, N.; Takahashi, M.; Matsui, M.; Ishii, S.; Date, T.; Sasamoto, S.; Ishizaki, R.: Isolation of human cDNA clones of MYB-related genes, A-MYB and B-MYB. Nucleic Acids Res. 16: 11075-11089, 1988.; and
Takahashi, T.; Nakagoshi, H.; Sarai, A.; Nomura, N.; Yamamoto, T.; Ishii, S.: Human A-myb gene encodes a transcriptional activator containing the negative regulatory domains. FEBS Lett.

Further studies establishing the function and utilities of MYBL1 are found in John Hopkins OMIM database record ID 159405, and in sited publications numbered 2527-2529 listed in the bibliography section hereinbelow, which are also hereby incorporated by reference. KIAA1028 (Accession XM_{—}166324) is another VGAM51 host target gene. KIAA1028 BINDING SITE is HOST TARGET binding site found in the 3′ untranslated region of mRNA encoded by KIAA1028, corresponding to a HOST TARGET binding site such as BINDING SITE I, BINDING SITE II or BINDING SITE III. Table 2 illustrates the complementarity of the nucleotide sequences of KIAA1028 BINDING SITE, designated SEQ ID:3547, to the nucleotide sequence of VGAM51 RNA, herein designated VGAM RNA, also designated SEQ ID:386.

Another function of VGAM51 is therefore inhibition of KIAA1028 (Accession XM_{—}166324). Accordingly, utilities of VGAM51 include diagnosis, prevention and treatment of diseases and clinical conditions associated with KIAA1028. MGC2488 (Accession NM_{—}024039) is another VGAM51 host target gene. MGC2488 BINDING SITE is HOST TARGET binding site found in the 3′ untranslated region of mRNA encoded by MGC2488, corresponding to a HOST TARGET binding site such as BINDING SITE I, BINDING SITE II or BINDING SITE III. Table 2 illustrates the complementarity of the nucleotide sequences of MGC2488 BINDING SITE, designated SEQ ID:2045, to the nucleotide sequence of VGAM51 RNA, herein designated VGAM RNA, also designated SEQ ID:386.

Another function of VGAM51 is therefore inhibition of MGC2488 (Accession NM_{—}024039). Accordingly, utilities of VGAM51 include diagnosis, prevention and treatment of diseases and clinical conditions associated with MGC2488. FIG. 1 further provides a conceptual description of a novel bioinformatically detected viral gene of the present invention, referred to here as Viral Genomic Address Messenger 52 (VGAM52) viral gene, which modulates expression of respective host target genes thereof, the function and utility of which host target genes is known in the art.

VGAM52 is a novel bioinformatically detected regulatory, non protein coding, viral micro RNA (miRNA) gene. The method by which VGAM52 was detected is described hereinabove with reference to FIGS. 1-8.

VGAM52 gene, herein designated VGAM GENE, is a viral gene contained in the genome of Vaccinia Virus. VGAM52 host target gene, herein designated VGAM HOST TARGET GENE, is a human gene contained in the human genome.

VGAM52 gene encodes a VGAM52 precursor RNA, herein designated VGAM PRECURSOR RNA. Similar to other miRNA genes, and unlike most ordinary genes, VGAM52 precursor RNA does not encode a protein. A nucleotide sequence identical or highly similar to the nucleotide sequence of VGAM52 precursor RNA is designated SEQ ID:38, and is provided hereinbelow with reference to the sequence listing part. Nucleotide sequence SEQ ID:38 is located at position 6573 relative to the genome of Vaccinia Virus.

VGAM52 precursor RNA folds onto itself, forming VGAM52 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM52 folded precursor RNA into VGAM52 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM52 RNA is designated SEQ ID:387, and is provided hereinbelow with reference to the sequence listing part.

VGAM52 host target gene, herein designated VGAM HOST TARGET GENE, encodes a corresponding messenger RNA, VGAM52 host target RNA, herein designated VGAM HOST TARGET RNA. VGAM52 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM52 RNA, herein designated VGAM RNA, binds complementarily to one or more host target binding sites located in untranslated regions of VGAM52 host target RNA, herein designated VGAM HOST TARGET RNA. This complementary binding is due to the fact that the nucleotide sequence of VGAM52 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM52 RNA, herein designated VGAM RNA, may have a different number of host target binding sites in untranslated regions of a VGAM52 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM52 RNA, herein designated VGAM RNA, to host target binding sites on VGAM52 host target RNA, herein designated VGAM HOST TARGET RNA, such as BINDING SITE I, BINDING SITE II and BINDING SITE III, inhibits translation of VGAM52 host target RNA into VGAM52 host target protein, herein designated VGAM HOST TARGET PROTEIN. VGAM host target protein is therefore outlined by a broken line.

It is appreciated that VGAM52 host target gene, herein designated VGAM HOST TARGET GENE, in fact represents a plurality of VGAM52 host target genes. The mRNA of each one of this plurality of VGAM52 host target genes comprises one or more host target binding sites, each having a nucleotide sequence which is at least partly complementary to VGAM52 RNA, herein designated VGAM RNA, and which when bound by VGAM52 RNA causes inhibition of translation of respective one or more VGAM52 host target proteins.

It is further appreciated by one skilled in the art that the mode of translational inhibition illustrated by FIG. 1 with specific reference to translational inhibition exerted by VGAM52 gene, herein designated VGAM GENE, on one or more VGAM52 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM52 is inhibition of expression of host target genes, as part of a novel viral mechanism of attacking a host. Accordingly, utilities of VGAM52 include diagnosis, prevention and treatment of viral infection by Vaccinia Virus. Specific functions, and accordingly utilities, of VGAM52 correlate with, and may be deduced from, the identity of the host target genes which VGAM52 binds and inhibits, and the function of these host target genes, as elaborated hereinbelow.

Nucleotide sequences of the VGAM52 precursor RNA, herein designated VGAM PRECURSOR RNA, and of the ‘diced’ VGAM52 RNA, herein designated VGAM RNA, and a schematic representation of the secondary folding of VGAM52 folded precursor RNA, herein designated VGAM FOLDED PRECURSOR RNA, of VGAM52 are further described hereinbelow with reference to Table 1.

Nucleotide sequences of host target binding sites, such as BINDING SITE-I, BINDING SITE-II and BINDING SITE-III of FIG. 1, found on VGAM52 host target RNA, and schematic representation of the complementarity of each of these host target binding sites to VGAM52 RNA, herein designated VGAM RNA, are described hereinbelow with reference to Table 2.

As mentioned hereinabove with reference to FIG. 1, a function of VGAM52 gene, herein designated VGAM is inhibition of expression of VGAM52 target genes. It is appreciated that specific functions, and accordingly utilities, of VGAM52 correlate with, and may be deduced from, the identity of the target genes which VGAM52 binds and inhibits, and the function of these target genes, as elaborated hereinbelow.

Chemokine (C-X3-C motif) Receptor 1 (CX3CR1, Accession XM_{—}047502) is a VGAM52 host target gene. CX3CR1 BINDING SITE is HOST TARGET binding site found in the 3′ untranslated region of mRNA encoded by CX3CR1, corresponding to a HOST TARGET binding site such as BINDING SITE I, BINDING SITE II or BINDING SITE III. Table 2 illustrates the complementarity of the nucleotide sequences of CX3CR1 BINDING SITE, designated SEQ ID:2891, to the nucleotide sequence of VGAM52 RNA, herein designated VGAM RNA, also designated SEQ ID:387.

A function of VGAM52 is therefore inhibition of Chemokine (C-X3-C motif) Receptor 1 (CX3CR1, Accession XM_{—}047502), a gene which mediates both the adhesive and migratory functions of fractalkine. Accordingly, utilities of VGAM52 include diagnosis, prevention and treatment of diseases and clinical conditions associated with CX3CR1. The function of CX3CR1 has been established by previous studies. Leukocyte trafficking at the endothelium requires both cellular adhesion molecules and chemotactic factors. Fractalkine (OMIM Ref. No. 601880), a transmembrane molecule with a CX3C-motif chemokine domain atop a mucin stalk, induces both adhesion and migration of leukocytes. Imai et al. (1997) identified a 7-transmembrane high-affinity receptor for fractalkine and showed that it mediates both the adhesive and migratory functions of fractalkine. The receptor, which the authors termed CX3CR1, requires pertussis toxin-sensitive G protein signaling to induce migration but not to support adhesion, which also occurs without other adhesion molecules but requires the architecture of a chemokine domain atop the mucin stalk. Natural killer cells predominantly express CX3CR1 and respond to fractalkine in both migration and adhesion. Imai et al. (1997) concluded that fractalkine and CX3CR1 represent new types of leukocyte trafficking regulators, performing both adhesive and chemotactic functions. CX3CR1 is an HIV coreceptor as well as a leukocyte chemotactic/adhesion receptor for fractalkine. Faure et al. (2000) identified 2 single nucleotide polymorphisms in the CX3CR1 gene in Caucasians and demonstrated that HIV-infected patients homozygous for 1249/M280 (601470.0001) progressed to AIDS more rapidly than those with other haplotypes (relative risk=2.13, P=0.039). Functional CX3CR1 analysis showed that fractalkine binding is reduced among patients homozygous for this particular haplotype. Thus, Faure et al. (2000) concluded that CX3CR1-1249/M280 is a recessive genetic risk factor for HIV/AIDS. Tripp et al. (2001) showed that the G glycoprotein of respiratory syncytial virus (RSV) shares a heparin-binding domain and a CX3C chemokine motif with CX3CL1. Binding analysis indicated that RSV can use CX3CR1 as a receptor. G glycoprotein binding mimics fractalkine binding and induces leukocyte chemotaxis. Tripp et al. (2001) concluded that RSV G glycoprotein uses its similarities with CX3C to facilitate infection and to modify the immune response.

Full details of the abovementioned studies are described in the following publications, the disclosure of which are hereby incorporated by reference:
Faure, S.; Meyer, L.; Costagliola, D.; Vaneensberghe, C.; Genin, E.; Autran, B.; French ALT and IMMUNOCO Study Groups; Delfraisay, J.-F.; SEROCO Study Group; McDermott, D. H.; Murphy, P. M.; Debre, P.; Theodorou, I.; Cambadiere, C.: Rapid progression to AIDS in HIV+ individuals with a structural variant of the chemokine receptor CX(3)CR1. Science 287: 2274-2277, 2000.; and
Moatti, D.; Faure, S.; Fumeron, F.; Amara, M. E. W.; Seknadji, P.; McDermott, D. H.; Debre, P.; Aumont, M. C.; Murphy, P. M.; de Prost, D.; Combadiere, C.: Polymorphism in the fractalk.

Further studies establishing the function and utilities of CX3CR1 are found in John Hopkins OMIM database record ID 601470, and in sited publications numbered 1552-155 and 1851-1556 listed in the bibliography section hereinbelow, which are also hereby incorporated by reference. Wingless-type MMTV Integration Site Family, Member 14 (WNT14, Accession NM_{—}003395) is another VGAM52 host target gene. WNT14 BINDING SITE is HOST TARGET binding site found in the 3′ untranslated region of mRNA encoded by WNT14, corresponding to a HOST TARGET binding site such as BINDING SITE I, BINDING SITE II or BINDING SITE III. Table 2 illustrates the complementarity of the nucleotide sequences of WNT14 BINDING SITE, designated SEQ ID: 1014, to the nucleotide sequence of VGAM52 RNA, herein designated VGAM RNA, also designated SEQ ID:387.

Another function of VGAM52 is therefore inhibition of Wingless-type MMTV Integration Site Family, Member 14 (WNT14, Accession NM_{—}003395), a gene which is a ligand for members of the frizzled family of seven transmembrane receptors and may be a signaling molecule which affect the development of discrete regions of tissues. Accordingly, utilities of VGAM52 include diagnosis, prevention and treatment of diseases and clinical conditions associated with WNT14. The function of WNT14 has been established by previous studies. The WNT gene family consists of structurally related genes that encode cysteine-rich secreted glycoproteins that act as extracellular signaling factors. WNT genes are implicated in a wide variety of biologic processes including cell fate determination and patterning in early embryos and in cell growth and/or differentiation in certain adult mammalian tissues. Bergstein et al. (1997) stated that all WNT genes identified in mammals encode proteins of approximately 350 to 400 amino acids with an amino-terminal secretory signal peptide followed by a short domain of low sequence conservation and then by a large conserved domain that includes 22 cysteine residues, the relative spacing of which is exactly conserved in the majority of known WNT proteins. By PCR with degenerate primers, Bergstein et al. (1997) isolated 2 novel members of the WNT family in humans, WNT14 and WNT15 (OMIM Ref. No. 602864). WNT14 shows 75% amino acid identity to chicken Wnt14 and 54% identity to human WNT15. Bergstein et al. (1997) reported that, based on both an unusual cysteine spacing pattern and amino acid sequence comparison, WNT14 and WNT15 are more closely related to Drosophila Wnt4 (OMIM Ref. No. 603490) and hagfish Wnt9 than to other members of the WNT family and may share common ancestry. During analysis of human genome draft sequence containing WNT3A (OMIM Ref. No. 606359), Saitoh et al. (2001) identified WNT14 gene fragments. Using RACE and cDNA-PCR, they assembled a WNT14 cDNA sequence. WNT14 encodes a deduced 365-amino acid protein containing an N-linked glycosylation site and residues conserved among members of the WNT family. Northern blot analysis detected a 4.4-kb WNT14 transcript at moderate levels in adult skeletal muscle and heart and at low levels in placenta, adult lung, pancreas, spleen, ovary, fetal brain, and fetal lung. Using cDNA-PCR, Saitoh et al. (2001) detected WNT14 expression in gastric cancer cell lines. Hartmann and Tabin (2001) presented evidence that Wnt14 plays a central role in initiating synovial joint formation in the chick limb. Wnt14 was expressed in joint-forming regions prior to the segmentation of the cartilage elements, and local misexpression of Wnt14 induced morphologic and molecular changes characteristic of the first steps of joint formation. Induction of an ectopic joint-like region by Wnt14 suppressed the formation of the immediately adjacent endogenous joint, potentially providing insight into the spacing of joints. Saitoh et al. (2001) determined that the WNT14 gene contains 4 exons and spans about 27 kb of genomic DNA. WNT14 and WNT3A are clustered in a head-to-head manner with an interval of about 58 kb.

Full details of the abovementioned studies are described in the following publications, the disclosure of which are hereby incorporated by reference:
Hartmann, C.; Tabin, C. J.: Wnt-14 plays a pivotal role in inducing synovial joint formation in the developing appendicular skeleton. Cell 104: 341-351, 2001.; and
Saitoh, T.; Hirai, M.; Katoh, M.: Molecular cloning and characterization of WNT3A and WNT14 clustered in human chromosome 1q42 region. Biochem. Biophys. Res. Commun. 284: 1168-1175, 20.

Further studies establishing the function and utilities of WNT14 are found in John Hopkins OMIM database record ID 602863, and in sited publications numbered 1210-1212 listed in the bibliography section hereinbelow, which are also hereby incorporated by reference. FLJ20275 (Accession NM_{—}017737) is another VGAM52 host target gene. FLJ20275 BINDING SITE is HOST TARGET binding site found in the 3′ untranslated region of mRNA encoded by FLJ20275, corresponding to a HOST TARGET binding site such as BINDING SITE I, BINDING SITE II or BINDING SITE III. Table 2 illustrates the complementarity of the nucleotide sequences of FLJ20275 BINDING SITE, designated SEQ ID: 1745, to the nucleotide sequence of VGAM52 RNA, herein designated VGAM RNA, also designated SEQ ID:387.

Another function of VGAM52 is therefore inhibition of FLJ20275 (Accession NM_{—}017737). Accordingly, utilities of VGAM52 include diagnosis, prevention and treatment of diseases and clinical conditions associated with FLJ20275. FLJ22390 (Accession NM_{—}022746) is another VGAM52 host target gene. FLJ22390 BINDING SITE is HOST TARGET binding site found in the 5′ untranslated region of mRNA encoded by FLJ22390, corresponding to a HOST TARGET binding site such as BINDING SITE I, BINDING SITE II or BINDING SITE III. Table 2 illustrates the complementarity of the nucleotide sequences of FLJ22390 BINDING SITE, designated SEQ ID:2002, to the nucleotide sequence of VGAM52 RNA, herein designated VGAM RNA, also designated SEQ ID:387.

Another function of VGAM52 is therefore inhibition of FLJ22390 (Accession NM_{—}022746). Accordingly, utilities of VGAM52 include diagnosis, prevention and treatment of diseases and clinical conditions associated with FLJ22390. FIG. 1 further provides a conceptual description of a novel bioinformatically detected viral gene of the present invention, referred to here as Viral Genomic Address Messenger 53 (VGAM53) viral gene, which modulates expression of respective host target genes thereof, the function and utility of which host target genes is known in the art.

VGAM53 is a novel bioinformatically detected regulatory, non protein coding, viral micro RNA (miRNA) gene. The method by which VGAM53 was detected is described hereinabove with reference to FIGS. 1-8.

VGAM53 gene, herein designated VGAM GENE, is a viral gene contained in the genome of Vaccinia Virus. VGAM53 host target gene, herein designated VGAM HOST TARGET GENE, is a human gene contained in the human genome.

VGAM53 gene encodes a VGAM53 precursor RNA, herein designated VGAM PRECURSOR RNA. Similar to other miRNA genes, and unlike most ordinary genes, VGAM53 precursor RNA does not encode a protein. A nucleotide sequence identical or highly similar to the nucleotide sequence of VGAM53 precursor RNA is designated SEQ ID:39, and is provided hereinbelow with reference to the sequence listing part. Nucleotide sequence SEQ ID:39 is located at position 5257 relative to the genome of Vaccinia Virus.

VGAM53 precursor RNA folds onto itself, forming VGAM53 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM53 folded precursor RNA into VGAM53 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM53 RNA is designated SEQ ID:388, and is provided hereinbelow with reference to the sequence listing part.

VGAM53 host target gene, herein designated VGAM HOST TARGET GENE, encodes a corresponding messenger RNA, VGAM53 host target RNA, herein designated VGAM HOST TARGET RNA. VGAM53 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM53 RNA, herein designated VGAM RNA, binds complementarily to one or more host target binding sites located in untranslated regions of VGAM53 host target RNA, herein designated VGAM HOST TARGET RNA. This complementary binding is due to the fact that the nucleotide sequence of VGAM53 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM53 RNA, herein designated VGAM RNA, may have a different number of host target binding sites in untranslated regions of a VGAM53 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM53 RNA, herein designated VGAM RNA, to host target binding sites on VGAM53 host target RNA, herein designated VGAM HOST TARGET RNA, such as BINDING SITE I, BINDING SITE II and BINDING SITE III, inhibits translation of VGAM53 host target RNA into VGAM53 host target protein, herein designated VGAM HOST TARGET PROTEIN. VGAM host target protein is therefore outlined by a broken line.

It is appreciated that VGAM53 host target gene, herein designated VGAM HOST TARGET GENE, in fact represents a plurality of VGAM53 host target genes. The mRNA of each one of this plurality of VGAM53 host target genes comprises one or more host target binding sites, each having a nucleotide sequence which is at least partly complementary to VGAM53 RNA, herein designated VGAM RNA, and which when bound by VGAM53 RNA causes inhibition of translation of respective one or more VGAM53 host target proteins.

It is further appreciated by one skilled in the art that the mode of translational inhibition illustrated by FIG. 1 with specific reference to translational inhibition exerted by VGAM53 gene, herein designated VGAM GENE, on one or more VGAM53 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM53 is inhibition of expression of host target genes, as part of a novel viral mechanism of attacking a host. Accordingly, utilities of VGAM53 include diagnosis, prevention and treatment of viral infection by Vaccinia Virus. Specific functions, and accordingly utilities, of VGAM53 correlate with, and may be deduced from, the identity of the host target genes which VGAM53 binds and inhibits, and the function of these host target genes, as elaborated hereinbelow.

Nucleotide sequences of the VGAM53 precursor RNA, herein designated VGAM PRECURSOR RNA, and of the ‘diced’ VGAM53 RNA, herein designated VGAM RNA, and a schematic representation of the secondary folding of VGAM53 folded precursor RNA, herein designated VGAM FOLDED PRECURSOR RNA, of VGAM53 are further described hereinbelow with reference to Table 1.

Nucleotide sequences of host target binding sites, such as BINDING SITE-I, BINDING SITE-II and BINDING SITE-III of FIG. 1, found on VGAM53 host target RNA, and schematic representation of the complementarity of each of these host target binding sites to VGAM53 RNA, herein designated VGAM RNA, are described hereinbelow with reference to Table 2.

As mentioned hereinabove with reference to FIG. 1, a function of VGAM53 gene, herein designated VGAM is inhibition of expression of VGAM53 target genes. It is appreciated that specific functions, and accordingly utilities, of VGAM53 correlate with, and may be deduced from, the identity of the target genes which VGAM53 binds and inhibits, and the function of these target genes, as elaborated hereinbelow.

LOC120864 (Accession XM_{—}058510) is a VGAM53 host target gene. LOC120864 BINDING SITE is HOST TARGET binding site found in the 5′ untranslated region of mRNA encoded by LOC120864, corresponding to a HOST TARGET binding site such as BINDING SITE I, BINDING SITE II or BINDING SITE III. Table 2 illustrates the complementarity of the nucleotide sequences of LOC120864 BINDING SITE, designated SEQ ID:2986, to the nucleotide sequence of VGAM53 RNA, herein designated VGAM RNA, also designated SEQ ID:388.

A function of VGAM53 is therefore inhibition of LOC120864 (Accession XM_{—}058510). Accordingly, utilities of VGAM53 include diagnosis, prevention and treatment of diseases and clinical conditions associated with LOC120864. FIG. 1 further provides a conceptual description of a novel bioinformatically detected viral gene of the present invention, referred to here as Viral Genomic Address Messenger 54 (VGAM54) viral gene, which modulates expression of respective host target genes thereof, the function and utility of which host target genes is known in the art.

VGAM54 is a novel bioinformatically detected regulatory, non protein coding, viral micro RNA (miRNA) gene. The method by which VGAM54 was detected is described hereinabove with reference to FIGS. 1-8.

VGAM54 gene, herein designated VGAM GENE, is a viral gene contained in the genome of Vaccinia Virus. VGAM54 host target gene, herein designated VGAM HOST TARGET GENE, is a human gene contained in the human genome.

VGAM54 gene encodes a VGAM54 precursor RNA, herein designated VGAM PRECURSOR RNA. Similar to other miRNA genes, and unlike most ordinary genes, VGAM54 precursor RNA does not encode a protein. A nucleotide sequence identical or highly similar to the nucleotide sequence of VGAM54 precursor RNA is designated SEQ ID:40, and is provided hereinbelow with reference to the sequence listing part. Nucleotide sequence SEQ ID:40 is located at position 4867 relative to the genome of Vaccinia Virus.

VGAM54 precursor RNA folds onto itself, forming VGAM54 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM54 folded precursor RNA into VGAM54 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM54 RNA is designated SEQ ID:389, and is provided hereinbelow with reference to the sequence listing part.

VGAM54 host target gene, herein designated VGAM HOST TARGET GENE, encodes a corresponding messenger RNA, VGAM54 host target RNA, herein designated VGAM HOST TARGET RNA. VGAM54 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM54 RNA, herein designated VGAM RNA, binds complementarily to one or more host target binding sites located in untranslated regions of VGAM54 host target RNA, herein designated VGAM HOST TARGET RNA. This complementary binding is due to the fact that the nucleotide sequence of VGAM54 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM54 RNA, herein designated VGAM RNA, may have a different number of host target binding sites in untranslated regions of a VGAM54 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM54 RNA, herein designated VGAM RNA, to host target binding sites on VGAM54 host target RNA, herein designated VGAM HOST TARGET RNA, such as BINDING SITE I, BINDING SITE II and BINDING SITE III, inhibits translation of VGAM54 host target RNA into VGAM54 host target protein, herein designated VGAM HOST TARGET PROTEIN. VGAM host target protein is therefore outlined by a broken line.

It is appreciated that VGAM54 host target gene, herein designated VGAM HOST TARGET GENE, in fact represents a plurality of VGAM54 host target genes. The mRNA of each one of this plurality of VGAM54 host target genes comprises one or more host target binding sites, each having a nucleotide sequence which is at least partly complementary to VGAM54 RNA, herein designated VGAM RNA, and which when bound by VGAM54 RNA causes inhibition of translation of respective one or more VGAM54 host target proteins.

It is further appreciated by one skilled in the art that the mode of translational inhibition illustrated by FIG. 1 with specific reference to translational inhibition exerted by VGAM54 gene, herein designated VGAM GENE, on one or more VGAM54 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM54 is inhibition of expression of host target genes, as part of a novel viral mechanism of attacking a host. Accordingly, utilities of VGAM54 include diagnosis, prevention and treatment of viral infection by Vaccinia Virus. Specific functions, and accordingly utilities, of VGAM54 correlate with, and may be deduced from, the identity of the host target genes which VGAM54 binds and inhibits, and the function of these host target genes, as elaborated hereinbelow.

Nucleotide sequences of the VGAM54 precursor RNA, herein designated VGAM PRECURSOR RNA, and of the ‘diced’ VGAM54 RNA, herein designated VGAM RNA, and a schematic representation of the secondary folding of VGAM54 folded precursor RNA, herein designated VGAM FOLDED PRECURSOR RNA, of VGAM54 are further described hereinbelow with reference to Table 1.

Nucleotide sequences of host target binding sites, such as BINDING SITE-I, BINDING SITE-II and BINDING SITE-III of FIG. 1, found on VGAM54 host target RNA, and schematic representation of the complementarity of each of these host target binding sites to VGAM54 RNA, herein designated VGAM RNA, are described hereinbelow with reference to Table 2.

As mentioned hereinabove with reference to FIG. 1, a function of VGAM54 gene, herein designated VGAM is inhibition of expression of VGAM54 target genes. It is appreciated that specific functions, and accordingly utilities, of VGAM54 correlate with, and may be deduced from, the identity of the target genes which VGAM54 binds and inhibits, and the function of these target genes, as elaborated hereinbelow.

KIAA0626 (Accession NM_{—}021647) is a VGAM54 host target gene. KIAA0626 BINDING SITE is HOST TARGET binding site found in the 3′ untranslated region of mRNA encoded by KIAA0626, corresponding to a HOST TARGET binding site such as BINDING SITE I, BINDING SITE II or BINDING SITE III. Table 2 illustrates the complementarity of the nucleotide sequences of KIAA0626 BINDING SITE, designated SEQ ID:1954, to the nucleotide sequence of VGAM54 RNA, herein designated VGAM RNA, also designated SEQ ID:389.

A function of VGAM54 is therefore inhibition of KIAA0626 (Accession NM_{—}021647). Accordingly, utilities of VGAM54 include diagnosis, prevention and treatment of diseases and clinical conditions associated with KIAA0626. KIAA1239 (Accession XM_{—}049078) is another VGAM54 host target gene. KIAA1239 BINDING SITE is HOST TARGET binding site found in the 3′ untranslated region of mRNA encoded by KIAA1239, corresponding to a HOST TARGET binding site such as BINDING SITE I, BINDING SITE II or BINDING SITE III. Table 2 illustrates the complementarity of the nucleotide sequences of KIAA1239 BINDING SITE, designated SEQ ID:2914, to the nucleotide sequence of VGAM54 RNA, herein designated VGAM RNA, also designated SEQ ID:389.

Another function of VGAM54 is therefore inhibition of KIAA1239 (Accession XM_{—}049078). Accordingly, utilities of VGAM54 include diagnosis, prevention and treatment of diseases and clinical conditions associated with KIAA1239. LOC201475 (Accession XM_{—}113967) is another VGAM54 host target gene. LOC201475 BINDING SITE is HOST TARGET binding site found in the 5′ untranslated region of mRNA encoded by LOC201475, corresponding to a HOST TARGET binding site such as BINDING SITE I, BINDING SITE II or BINDING SITE III. Table 2 illustrates the complementarity of the nucleotide sequences of LOC201475 BINDING SITE, designated SEQ ID:3428, to the nucleotide sequence of VGAM54 RNA, herein designated VGAM RNA, also designated SEQ ID:389.

Another function of VGAM54 is therefore inhibition of LOC201475 (Accession XM_{—}113967). Accordingly, utilities of VGAM54 include diagnosis, prevention and treatment of diseases and clinical conditions associated with LOC201475. FIG. 1 further provides a conceptual description of a novel bioinformatically detected viral gene of the present invention, referred to here as Viral Genomic Address Messenger 55 (VGAM55) viral gene, which modulates expression of respective host target genes thereof, the function and utility of which host target genes is known in the art.

VGAM55 is a novel bioinformatically detected regulatory, non protein coding, viral micro RNA (miRNA) gene. The method by which VGAM55 was detected is described hereinabove with reference to FIGS. 1-8.

VGAM55 gene, herein designated VGAM GENE, is a viral gene contained in the genome of Vaccinia Virus. VGAM55 host target gene, herein designated VGAM HOST TARGET GENE, is a human gene contained in the human genome.

VGAM55 gene encodes a VGAM55 precursor RNA, herein designated VGAM PRECURSOR RNA. Similar to other miRNA genes, and unlike most ordinary genes, VGAM55 precursor RNA does not encode a protein. A nucleotide sequence identical or highly similar to the nucleotide sequence of VGAM55 precursor RNA is designated SEQ ID:41, and is provided hereinbelow with reference to the sequence listing part. Nucleotide sequence SEQ ID:41 is located at position 185510 relative to the genome of Vaccinia Virus.

VGAM55 precursor RNA folds onto itself, forming VGAM55 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM55 folded precursor RNA into VGAM55 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM55 RNA is designated SEQ ID:390, and is provided hereinbelow with reference to the sequence listing part.

VGAM55 host target gene, herein designated VGAM HOST TARGET GENE, encodes a corresponding messenger RNA, VGAM55 host target RNA, herein designated VGAM HOST TARGET RNA. VGAM55 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM55 RNA, herein designated VGAM RNA, binds complementarily to one or more host target binding sites located in untranslated regions of VGAM55 host target RNA, herein designated VGAM HOST TARGET RNA. This complementary binding is due to the fact that the nucleotide sequence of VGAM55 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM55 RNA, herein designated VGAM RNA, may have a different number of host target binding sites in untranslated regions of a VGAM55 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM55 RNA, herein designated VGAM RNA, to host target binding sites on VGAM55 host target RNA, herein designated VGAM HOST TARGET RNA, such as BINDING SITE I, BINDING SITE II and BINDING SITE III, inhibits translation of VGAM55 host target RNA into VGAM55 host target protein, herein designated VGAM HOST TARGET PROTEIN. VGAM host target protein is therefore outlined by a broken line.

It is appreciated that VGAM55 host target gene, herein designated VGAM HOST TARGET GENE, in fact represents a plurality of VGAM55 host target genes. The mRNA of each one of this plurality of VGAM55 host target genes comprises one or more host target binding sites, each having a nucleotide sequence which is at least partly complementary to VGAM55 RNA, herein designated VGAM RNA, and which when bound by VGAM55 RNA causes inhibition of translation of respective one or more VGAM55 host target proteins.

It is further appreciated by one skilled in the art that the mode of translational inhibition illustrated by FIG. 1 with specific reference to translational inhibition exerted by VGAM55 gene, herein designated VGAM GENE, on one or more VGAM55 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM55 is inhibition of expression of host target genes, as part of a novel viral mechanism of attacking a host. Accordingly, utilities of VGAM55 include diagnosis, prevention and treatment of viral infection by Vaccinia Virus. Specific functions, and accordingly utilities, of VGAM55 correlate with, and may be deduced from, the identity of the host target genes which VGAM55 binds and inhibits, and the function of these host target genes, as elaborated hereinbelow.

Nucleotide sequences of the VGAM55 precursor RNA, herein designated VGAM PRECURSOR RNA, and of the ‘diced’ VGAM55 RNA, herein designated VGAM RNA, and a schematic representation of the secondary folding of VGAM55 folded precursor RNA, herein designated VGAM FOLDED PRECURSOR RNA, of VGAM55 are further described hereinbelow with reference to Table 1.

Nucleotide sequences of host target binding sites, such as BINDING SITE-I, BINDING SITE-II and BINDING SITE-III of FIG. 1, found on VGAM55 host target RNA, and schematic representation of the complementarity of each of these host target binding sites to VGAM55 RNA, herein designated VGAM RNA, are described hereinbelow with reference to Table 2.

As mentioned hereinabove with reference to FIG. 1, a function of VGAM55 gene, herein designated VGAM is inhibition of expression of VGAM55 target genes. It is appreciated that specific functions, and accordingly utilities, of VGAM55 correlate with, and may be deduced from, the identity of the target genes which VGAM55 binds and inhibits, and the function of these target genes, as elaborated hereinbelow.

Archain 1 (ARCN1, Accession NM_{—}001655) is a VGAM55 host target gene. ARCN1 BINDING SITE is HOST TARGET binding site found in the 3′ untranslated region of mRNA encoded by ARCN1, corresponding to a HOST TARGET binding site such as BINDING SITE I, BINDING SITE II or BINDING SITE III. Table 2 illustrates the complementarity of the nucleotide sequences of ARCN1 BINDING SITE, designated SEQ ID:844, to the nucleotide sequence of VGAM55 RNA, herein designated VGAM RNA, also designated SEQ ID:390.

A function of VGAM55 is therefore inhibition of Archain 1 (ARCN1, Accession NM_{—}001655), a gene which plays a fundamental role in eukaryotic cell biology. Accordingly, utilities of VGAM55 include diagnosis, prevention and treatment of diseases and clinical conditions associated with ARCN1. The function of ARCN1 has been established by previous studies. Radice et al. (1995) identified a gene that maps approximately 50-kb telomeric to MLL (OMIM Ref. No. 159555) in band 11q23.3, a locus disrupted in certain leukemia-associated translocation chromosomes. A 200-kb genomic fragment from a YAC that includes MLL was used to screen a cDNA library of the R54;11 cell line which carries a translocation chromosome t(4;11)(q21;q23). The cDNA sequence predicts a 511-amino acid protein which shares similarity with predicted proteins of unknown function from rice (Oryza sativa) and Drosophila. Because of this ancient conservation the authors proposed the name archain (ARCN1). Radice et al. (1995) detected 4-kb ARCN1 transcripts by Northern blot analysis in all tissues examined. The protein encoded by the ARCN1 gene, the coatomer protein delta-COP, probably plays a fundamental role in eukaryotic cell biology. Tunnacliffe at al. (1996) demonstrated that it is conserved across diverse eukaryotes. Very close or identical matches were seen in rat and cow; highly significant matches were seen with 2 plant species, A. thaliana (cress) and S. tuberosum (OMIM Ref. No. potato). Of particular biologic significance was the match with a sequence on yeast chromosome VI, from which Tunnacliffe et al. (1996) were able to determine the yeast archain gene and protein sequence. Unpublished data indicated that in situ hybridizations on mouse embryo sections showed archain transcripts throughout the whole animal.

Full details of the abovementioned studies are described in the following publications, the disclosure of which are hereby incorporated by reference:
Radice, P.; Pensotti, V.; Jones, C.; Perry, H.; Pierotti, M. A.; Tunnacliffe, A.: The human archain gene, ARCN1, has highly conserved homologs in rice and Drosophila. Genomics 26: 101-106, 1995.; and
Tunnacliffe, A.; van de Vrugt, H.; Pensotti, V.; Radice, P.: The coatomer protein delta-COP, encoded by the archain gene, is conserved across diverse eukaryotes. Mammalian Genome 7: 78.

Further studies establishing the function and utilities of ARCN1 are found in John Hopkins OMIM database record ID 600820, and in sited publications numbered 1698-1699 listed in the bibliography section hereinbelow, which are also hereby incorporated by reference. Cathepsin K (pycnodysostosis) (CTSK, Accession NM_{—}000396) is another VGAM55 host target gene. CTSK BINDING SITE is HOST TARGET binding site found in the 3′ untranslated region of mRNA encoded by CTSK, corresponding to a HOST TARGET binding site such as BINDING SITE I, BINDING SITE II or BINDING SITE III. Table 2 illustrates the complementarity of the nucleotide sequences of CTSK BINDING SITE, designated SEQ ID:736, to the nucleotide sequence of VGAM55 RNA, herein designated VGAM RNA, also designated SEQ ID:390.

Another function of VGAM55 is therefore inhibition of Cathepsin K (pycnodysostosis) (CTSK, Accession NM_{—}000396). Accordingly, utilities of VGAM55 include diagnosis, prevention and treatment of diseases and clinical conditions associated with CTSK. High-mobility Group 20A (HMG20A, Accession NM_{—}018200) is another VGAM55 host target gene. HMG20A BINDING SITE is HOST TARGET binding site found in the 3′ untranslated region of mRNA encoded by HMG20A, corresponding to a HOST TARGET binding site such as BINDING SITE I, BINDING SITE II or BINDING SITE III. Table 2 illustrates the complementarity of the nucleotide sequences of HMG20A BINDING SITE, designated SEQ ID:1800, to the nucleotide sequence of VGAM55 RNA, herein designated VGAM RNA, also designated SEQ ID:390.

Another function of VGAM55 is therefore inhibition of High-mobility Group 20A (HMG20A, Accession NM_{—}018200). Accordingly, utilities of VGAM55 include diagnosis, prevention and treatment of diseases and clinical conditions associated with HMG20A. PR Domain Containing 2, with ZNF Domain (PRDM2, Accession NM_{—}012231) is another VGAM55 host target gene. PRDM2 BINDING SITE is HOST TARGET binding site found in the 3′ untranslated region of mRNA encoded by PRDM2, corresponding to a HOST TARGET binding site such as BINDING SITE I, BINDING SITE II or BINDING SITE III. Table 2 illustrates the complementarity of the nucleotide sequences of PRDM2 BINDING SITE, designated SEQ ID: 1415, to the nucleotide sequence of VGAM55 RNA, herein designated VGAM RNA, also designated SEQ ID:390.

Another function of VGAM55 is therefore inhibition of PR Domain Containing 2, with ZNF Domain (PRDM2, Accession NM_{—}012231), a gene which plays a role in transcriptional regulation during neuronal differentiation and pathogenesis of retinoblastoma. Accordingly, utilities of VGAM55 include diagnosis, prevention and treatment of diseases and clinical conditions associated with PRDM2. The function of PRDM2 has been established by previous studies. The retinoblastoma protein (OMIM Ref. No. 180200) is a target of viral oncoproteins. To explore the hypothesis that viral proteins may be structural mimics of cellular RB-binding proteins that normally mediate RB function, Buyse et al. (1995) searched cDNA libraries for RB-binding proteins. They reported the cloning of a cDNA for the zinc finger protein they called RIZ from rat and human cells. It is a 250-kD nuclear protein containing 8 zinc finger motifs. It contains an RB-binding motif that is related to that of the adenovirus E1A oncoprotein; RIZ also shares an antigenic epitope with the C terminus of E1A. It is expressed in human retinoblastoma cells and at low levels in all other human cell lines examined. While the function of RIZ was not clear, its structure and pattern of expression suggested to Buyse et al. (1995) a role in transcriptional regulation during neuronal differentiation and pathogenesis of retinoblastoma. The distal portion of chromosome 1p is one of the most commonly affected regions in human cancer. In a study of hereditary and sporadic colorectal cancer, Chadwick et al. (2000) identified a region of frequent deletion at 32.2 centimorgans from 1ptel. Deletion breakpoints clustered in the vicinity of or inside the gene RIZ. Sequence analysis demonstrated frequent frameshift mutations of the RIZ gene. The mutations consisted of 1- or 2-bp deletions of coding poly(A) tracts (A)₈ or (A)₉, and were confined to microsatellite-unstable colorectal tumors, being present in 9 of 24 (37.5%) primary tumors and in 6 of 11 (54.5%) cell lines; in 2 cell lines the mutation was homozygous (or hemizygous). The mutations apparently were selected clonally in tumorigenesis, because similar poly(A) tracts in other genes were not affected. Of the 2 alternative products of the gene, RIZ1 contains a PR domain implicated in tumor suppressor function and RIZ2 lacks this motif. Chadwick et al. (2000) proposed that RIZ is a target of observed 1p alterations, with impairment of the PR domain-mediated function through either frameshift mutation or genomic deletion.

Full details of the abovementioned studies are described in the following publications, the disclosure of which are hereby incorporated by reference:
Buyse, I. M.; Shao, G.; Huang, S.: The retinoblastoma protein binds to RIZ, a zinc-finger protein that shares an epitope with the adenovirus E1A protein. Proc. Nat. Acad. Sci. 92: 4467-4471, 1995.; and
Chadwick, R. B.; Jiang, G.-L.; Bennington, G. A.; Yuan, B.; Johnson, C. K.; Stevens, M. W.; Niemann, T. H.; Peltomaki, P.; Huang, S.; de la Chapelle, A.: Candidate tumor suppressor RIZ.

Further studies establishing the function and utilities of PRDM2 are found in John Hopkins OMIM database record ID 601196, and in sited publications numbered 2227-2231 listed in the bibliography section hereinbelow, which are also hereby incorporated by reference. RNA (guanine-7-) Methyltransferase (RNMT, Accession NM_{—}003799) is another VGAM55 host target gene. RNMT BINDING SITE is HOST TARGET binding site found in the 3′ untranslated region of mRNA encoded by RNMT, corresponding to a HOST TARGET binding site such as BINDING SITE I, BINDING SITE II or BINDING SITE III. Table 2 illustrates the complementarity of the nucleotide sequences of RNMT BINDING SITE, designated SEQ ID: 1060, to the nucleotide sequence of VGAM55 RNA, herein designated VGAM RNA, also designated SEQ ID:390.

Another function of VGAM55 is therefore inhibition of RNA (guanine-7-) Methyltransferase (RNMT, Accession NM_{—}003799), a gene which catalyzes the methylation of GpppN- at the guanine N7 position. Accordingly, utilities of VGAM55 include diagnosis, prevention and treatment of diseases and clinical conditions associated with RNMT. The function of RNMT has been established by previous studies. In mammals, 5-prime-terminal caps are formed on nascent pre-mRNAs by the sequential action of 2 enzymes, the bifunctional capping enzyme RNGTT (OMIM Ref. No. 603512) and RNA (guanine-7) methyltransferase. RNGTT catalyzes the removal of the gamma-phosphate of the initiating nucleotide and transfers GMP from GTP to the resulting diphosphate end. RNA (guanine-7) methyltransferase catalyzes the subsequent N7 methylation of the newly formed termini. The terminal 7-methylguanosine is recognized by cap-binding proteins that facilitate key events in gene expression. By searching an EST database for sequences homologous to that of S. cerevisiae RNA (guanine-7) methyltransferase, Pillutla et al. (1998) identified a human Met cDNA. The predicted 476-amino acid MET protein contains several conserved motifs known to be required for methyltransferase activity. Recombinant Met exhibited RNA (guanine-7) methyltransferase activity in vitro, and formed ternary complexes with RNGTT and the elongating form of RNA polymerase II. By screening human brain cDNAs for those encoding large proteins, Ishikawa et al. (1997) identified KIAA0398, an RNMT cDNA. Tsukamoto et al. (1998) isolated 3 human cDNAs encoding mRNA RNMT, which they termed HCMT1a, HCMT1b, and HCMT1c, which appear to be produced by alternative splicing. HCMT1a and HCMT1b encode deduced proteins of 476 and 504 amino acids, respectively, and differ only in the region encoding the C-terminal portion of the enzyme after residue 465. HCMT1c appears to encode the same polypeptide as HCMT1a; however, the 3-prime noncoding region of HCMT1c contains sequences corresponding to portions of both HCMT1a and HCMT1b. RT-PCR detected expression of the 3 mRNAs in all tissues tested. Recombinant HCMT1a expressed in E. coli exhibited mRNA RNMT activity, whereas recombinant HCMT1b did not. By analysis of a radiation hybrid panel, Pillutla et al. (1998) and Ishikawa et al. (1997) mapped the RNMT gene to chromosome 18. Pillutla et al. (1998) refined the location to 18p11.23-p11.22 using fluorescence in situ hybridization.

Full details of the abovementioned studies are described in the following publications, the disclosure of which are hereby incorporated by reference:
Pillutla, R. C.; Yue, Z.; Maldonado, E.; Shatkin, A. J.: Recombinant human mRNA cap methyltransferase binds capping enzyme/RNA polymerase IIo complexes. J. Biol. Chem. 273: 21443-21446, 1998.; and
Tsukamoto, T.; Shibagaki, Y.; Niikura, Y.; Mizumoto, K.: Cloning and characterization of three human cDNAs encoding mRNA (guanine-7)-methyltransferase, an mRNA cap methylase. Biochem. B.

Further studies establishing the function and utilities of RNMT are found in John Hopkins OMIM database record ID 603514, and in sited publications numbered 25 and 258-259 listed in the bibliography section hereinbelow, which are also hereby incorporated by reference. Src Family Associated Phosphoprotein 1 (SCAP1, Accession NM_{—}003726) is another VGAM55 host target gene. SCAP1 BINDING SITE is HOST TARGET binding site found in the 3′ untranslated region of mRNA encoded by SCAP1, corresponding to a HOST TARGET binding site such as BINDING SITE I, BINDING SITE II or BINDING SITE III. Table 2 illustrates the complementarity of the nucleotide sequences of SCAP1 BINDING SITE, designated SEQ ID:1052, to the nucleotide sequence of VGAM55 RNA, herein designated VGAM RNA, also designated SEQ ID:390.

Another function of VGAM55 is therefore inhibition of Src Family Associated Phosphoprotein 1 (SCAP1, Accession NM_{—}003726). Accordingly, utilities of VGAM55 include diagnosis, prevention and treatment of diseases and clinical conditions associated with SCAP1. Usher Syndrome 3A (USH3A, Accession NM_{—}052995) is another VGAM55 host target gene. USH3A BINDING SITE is HOST TARGET binding site found in the 5′ untranslated region of mRNA encoded by USH3A, corresponding to a HOST TARGET binding site such as BINDING SITE I, BINDING SITE II or BINDING SITE III. Table 2 illustrates the complementarity of the nucleotide sequences of USH3A BINDING SITE, designated SEQ ID:2355, to the nucleotide sequence of VGAM55 RNA, herein designated VGAM RNA, also designated SEQ ID:390.

Another function of VGAM55 is therefore inhibition of Usher Syndrome 3A (USH3A, Accession NM_{—}052995). Accordingly, utilities of VGAM55 include diagnosis, prevention and treatment of diseases and clinical conditions associated with USH3A. Vesicle Amine Transport Protein 1 Homolog (T californica) (VAT1, Accession NM_{—}006373) is another VGAM55 host target gene. VAT1 BINDING SITE is HOST TARGET binding site found in the 3′ untranslated region of mRNA encoded by VAT1, corresponding to a HOST TARGET binding site such as BINDING SITE I, BINDING SITE II or BINDING SITE III. Table 2 illustrates the complementarity of the nucleotide sequences of VAT1 BINDING SITE, designated SEQ ID:1292, to the nucleotide sequence of VGAM55 RNA, herein designated VGAM RNA, also designated SEQ ID:390.

Another function of VGAM55 is therefore inhibition of Vesicle Amine Transport Protein 1 Homolog (T californica) (VAT1, Accession NM_{—}006373), a gene which is a membrane protein of cholinergic synaptic vesicles. Accordingly, utilities of VGAM55 include diagnosis, prevention and treatment of diseases and clinical conditions associated with VAT1. The function of VAT1 has been established by previous studies. The physiologic and behavioral effects of pharmacologic agents that interfere with the transport of monoamine neurotransmitters into vesicles suggest that vesicular amine transport may contribute to human neuropsychiatric disease. Biogenic amines have been implicated in a wide range of clinical disorders and physiologic states such as consciousness, motivation, organizational thought, mood, and motor control, sensory perception, and autonomic phenomena such as heart rate, vascular tone, and blood pressure. Peter et al. (1993) isolated a human cDNA for the brain vesicular amine transporter. They found that the brain synaptic vesicle amine transporter (SVAT) showed conservation with the corresponding gene in the rat in the regions that diverge extensively between rat SVAT and the rat adrenal chromaffin granule amine transporter (CGAT). Using the cloned sequences with a panel of mouse/human hybrids and in situ hybridization, Peter et al. (1993) mapped the adrenal CGAT gene (VMAT1) to 8p21.3 and the brain SVAT gene (OMIM Ref. No. 193001) to 10q25. This gene is also symbolized as SLC18A1. Roghani et al. (1996) showed that the mouse Slc18a1 gene maps to mouse chromosome 8 by linkage analysis.

Full details of the abovementioned studies are described in the following publications, the disclosure of which are hereby incorporated by reference:
Peter, D.; Finn, J. P.; Klisak, I.; Liu, Y.; Kojis, T.; Heinzmann, C.; Roghani, A.; Sparkes, R. S.; Edwards, R. H.: Chromosomal localization of the human vesicular amine transporter genes. Genomics 18: 720-723, 1993.; and
Roghani, A.; Welch, C.; Xia, Y.-R.; Liu, Y.; Peter, D.; Finn, J. P.; Edwards, R. H.; Lusis, A. J.: Assignment of the mouse vesicular monoamine transporter genes, Slc18a1 and Slc18a2, t.

Further studies establishing the function and utilities of VAT1 are found in John Hopkins OMIM database record ID 193002, and in sited publications numbered 2400-2401 listed in the bibliography section hereinbelow, which are also hereby incorporated by reference. Allograft Inflammatory Factor 1 (AIF1, Accession NM_{—}032955) is another VGAM55 host target gene. AIF1 BINDING SITE is HOST TARGET binding site found in the 5′ untranslated region of mRNA encoded by AIF1, corresponding to a HOST TARGET binding site such as BINDING SITE I, BINDING SITE II or BINDING SITE III. Table 2 illustrates the complementarity of the nucleotide sequences of AIF1 BINDING SITE, designated SEQ ID:2297, to the nucleotide sequence of VGAM55 RNA, herein designated VGAM RNA, also designated SEQ ID:390.

Another function of VGAM55 is therefore inhibition of Allograft Inflammatory Factor 1 (AIF1, Accession NM_{—}032955). Accordingly, utilities of VGAM55 include diagnosis, prevention and treatment of diseases and clinical conditions associated with AIF1. Complexin 1 (CPLX1, Accession NM_{—}006651) is another VGAM55 host target gene. CPLX1 BINDING SITE is HOST TARGET binding site found in the 3′ untranslated region of mRNA encoded by CPLX1, corresponding to a HOST TARGET binding site such as BINDING SITE I, BINDING SITE II or BINDING SITE III. Table 2 illustrates the complementarity of the nucleotide sequences of CPLX1 BINDING SITE, designated SEQ ID:1317, to the nucleotide sequence of VGAM55 RNA, herein designated VGAM RNA, also designated SEQ ID:390.

Another function of VGAM55 is therefore inhibition of Complexin 1 (CPLX1, Accession NM_{—}006651). Accordingly, utilities of VGAM55 include diagnosis, prevention and treatment of diseases and clinical conditions associated with CPLX1. CXYorf1 (Accession XM_{—}088704) is another VGAM55 host target gene. CXYorf1 BINDING SITE is HOST TARGET binding site found in the 3′ untranslated region of mRNA encoded by CXYorf1, corresponding to a HOST TARGET binding site such as BINDING SITE I, BINDING SITE II or BINDING SITE III. Table 2 illustrates the complementarity of the nucleotide sequences of CXYorf1 BINDING SITE, designated SEQ ID:3228, to the nucleotide sequence of VGAM55 RNA, herein designated VGAM RNA, also designated SEQ ID:390.

Another function of VGAM55 is therefore inhibition of CXYorf1 (Accession XM_{—}088704). Accordingly, utilities of VGAM55 include diagnosis, prevention and treatment of diseases and clinical conditions associated with CXYorf1. FLJ12221 (Accession XM_{—}031342) is another VGAM55 host target gene. FLJ12221 BINDING SITE is HOST TARGET binding site found in the 3′ untranslated region of mRNA encoded by FLJ12221, corresponding to a HOST TARGET binding site such as BINDING SITE I, BINDING SITE II or BINDING SITE III. Table 2 illustrates the complementarity of the nucleotide sequences of FLJ12221 BINDING SITE, designated SEQ ID:2637, to the nucleotide sequence of VGAM55 RNA, herein designated VGAM RNA, also designated SEQ ID:390.

Another function of VGAM55 is therefore inhibition of FLJ12221 (Accession XM_{—}031342). Accordingly, utilities of VGAM55 include diagnosis, prevention and treatment of diseases and clinical conditions associated with FLJ12221. FLJ20207 (Accession NM_{—}017711) is another VGAM55 host target gene. FLJ20207 BINDING SITE is HOST TARGET binding site found in the 3′ untranslated region of mRNA encoded by FLJ20207, corresponding to a HOST TARGET binding site such as BINDING SITE I, BINDING SITE II or BINDING SITE III. Table 2 illustrates the complementarity of the nucleotide sequences of FLJ20207 BINDING SITE, designated SEQ ID:1741, to the nucleotide sequence of VGAM55 RNA, herein designated VGAM RNA, also designated SEQ ID:390.

Another function of VGAM55 is therefore inhibition of FLJ20207 (Accession NM_{—}017711). Accordingly, utilities of VGAM55 include diagnosis, prevention and treatment of diseases and clinical conditions associated with FLJ20207. FLJ22215 (Accession XM_{—}173021) is another VGAM55 host target gene. FLJ22215 BINDING SITE is HOST TARGET binding site found in the 3′ untranslated region of mRNA encoded by FLJ22215, corresponding to a HOST TARGET binding site such as BINDING SITE I, BINDING SITE II or BINDING SITE III. Table 2 illustrates the complementarity of the nucleotide sequences of FLJ22215 BINDING SITE, designated SEQ ID:3715, to the nucleotide sequence of VGAM55 RNA, herein designated VGAM RNA, also designated SEQ ID:390.

Another function of VGAM55 is therefore inhibition of FLJ22215 (Accession XM_{—}173021). Accordingly, utilities of VGAM55 include diagnosis, prevention and treatment of diseases and clinical conditions associated with FLJ22215. FLJ31547 (Accession NM_{—}145024) is another VGAM55 host target gene. FLJ31547 BINDING SITE is HOST TARGET binding site found in the 3′ untranslated region of mRNA encoded by FLJ31547, corresponding to a HOST TARGET binding site such as BINDING SITE I, BINDING SITE II or BINDING SITE III. Table 2 illustrates the complementarity of the nucleotide sequences of FLJ31547 BINDING SITE, designated SEQ ID:2511, to the nucleotide sequence of VGAM55 RNA, herein designated VGAM RNA, also designated SEQ ID:390.

Another function of VGAM55 is therefore inhibition of FLJ31547 (Accession NM_{—}145024). Accordingly, utilities of VGAM55 include diagnosis, prevention and treatment of diseases and clinical conditions associated with FLJ31547. HSCBCIP1 (Accession XM_{—}114210) is another VGAM55 host target gene. HSCBCIP1 BINDING SITE is HOST TARGET binding site found in the 3′ untranslated region of mRNA encoded by HSCBCIP1, corresponding to a HOST TARGET binding site such as BINDING SITE I, BINDING SITE II or BINDING SITE III. Table 2 illustrates the complementarity of the nucleotide sequences of HSCBCIP1 BINDING SITE, designated SEQ ID:3449, to the nucleotide sequence of VGAM55 RNA, herein designated VGAM RNA, also designated SEQ ID:390.

Another function of VGAM55 is therefore inhibition of HSCBCIP1 (Accession XM_{—}114210). Accordingly, utilities of VGAM55 include diagnosis, prevention and treatment of diseases and clinical conditions associated with HSCBCIP1. KIAA0515 (Accession XM_{—}033380) is another VGAM55 host target gene. KIAA0515 BINDING SITE is HOST TARGET binding site found in the 5′ untranslated region of mRNA encoded by KIAA0515, corresponding to a HOST TARGET binding site such as BINDING SITE I, BINDING SITE II or BINDING SITE III. Table 2 illustrates the complementarity of the nucleotide sequences of KIAA0515 BINDING SITE, designated SEQ ID:2670, to the nucleotide sequence of VGAM55 RNA, herein designated VGAM RNA, also designated SEQ ID:390.

Another function of VGAM55 is therefore inhibition of KIAA0515 (Accession XM_{—}033380). Accordingly, utilities of VGAM55 include diagnosis, prevention and treatment of diseases and clinical conditions associated with KIAA0515. KIAA1514 (Accession NM_{—}019064) is another VGAM55 host target gene. KIAA1514 BINDING SITE is HOST TARGET binding site found in the 3′ untranslated region of mRNA encoded by KIAA1514, corresponding to a HOST TARGET binding site such as BINDING SITE I, BINDING SITE II or BINDING SITE III. Table 2 illustrates the complementarity of the nucleotide sequences of KIAA1514 BINDING SITE, designated SEQ ID:1877, to the nucleotide sequence of VGAM55 RNA, herein designated VGAM RNA, also designated SEQ ID:390.

Another function of VGAM55 is therefore inhibition of KIAA1514 (Accession NM_{—}019064). Accordingly, utilities of VGAM55 include diagnosis, prevention and treatment of diseases and clinical conditions associated with KIAA1514. KIAA1553 (Accession XM_{—}166320) is another VGAM55 host target gene. KIAA1553 BINDING SITE is HOST TARGET binding site found in the 3′ untranslated region of mRNA encoded by KIAA1553, corresponding to a HOST TARGET binding site such as BINDING SITE I, BINDING SITE II or BINDING SITE III. Table 2 illustrates the complementarity of the nucleotide sequences of KIAA1553 BINDING SITE, designated SEQ ID:3544, to the nucleotide sequence of VGAM55 RNA, herein designated VGAM RNA, also designated SEQ ID:390.

Another function of VGAM55 is therefore inhibition of KIAA1553 (Accession XM_{—}166320). Accordingly, utilities of VGAM55 include diagnosis, prevention and treatment of diseases and clinical conditions associated with KIAA1553. KIAA1656 (Accession XM_{—}038022) is another VGAM55 host target gene. KIAA1656 BINDING SITE is HOST TARGET binding site found in the 3′ untranslated region of mRNA encoded by KIAA1656, corresponding to a HOST TARGET binding site such as BINDING SITE I, BINDING SITE II or BINDING SITE III. Table 2 illustrates the complementarity of the nucleotide sequences of KIAA1656 BINDING SITE, designated SEQ ID:2730, to the nucleotide sequence of VGAM55 RNA, herein designated VGAM RNA, also designated SEQ ID:390.

Another function of VGAM55 is therefore inhibition of KIAA1656 (Accession XM_{—}038022). Accordingly, utilities of VGAM55 include diagnosis, prevention and treatment of diseases and clinical conditions associated with KIAA1656. KIAA1877 (Accession XM_{—}038616) is another VGAM55 host target gene. KIAA1877 BINDING SITE is HOST TARGET binding site found in the 3′ untranslated region of mRNA encoded by KIAA1877, corresponding to a HOST TARGET binding site such as BINDING SITE I, BINDING SITE II or BINDING SITE III. Table 2 illustrates the complementarity of the nucleotide sequences of KIAA1877 BINDING SITE, designated SEQ ID:2741, to the nucleotide sequence of VGAM55 RNA, herein designated VGAM RNA, also designated SEQ ID:390.

Another function of VGAM55 is therefore inhibition of KIAA1877 (Accession XM_{—}038616). Accordingly, utilities of VGAM55 include diagnosis, prevention and treatment of diseases and clinical conditions associated with KIAA1877. MTCH1 (Accession NM_{—}014341) is another VGAM55 host target gene. MTCH1 BINDING SITE is HOST TARGET binding site found in the 3′ untranslated region of mRNA encoded by MTCH1, corresponding to a HOST TARGET binding site such as BINDING SITE I, BINDING SITE II or BINDING SITE III. Table 2 illustrates the complementarity of the nucleotide sequences of MTCH1 BINDING SITE, designated SEQ ID: 1493, to the nucleotide sequence of VGAM55 RNA, herein designated VGAM RNA, also designated SEQ ID:390.

Another function of VGAM55 is therefore inhibition of MTCH1 (Accession NM_{—}014341). Accordingly, utilities of VGAM55 include diagnosis, prevention and treatment of diseases and clinical conditions associated with MTCH1. N-PAC (Accession XM_{—}048113) is another VGAM55 host target gene. N-PAC BINDING SITE is HOST TARGET binding site found in the 3′ untranslated region of mRNA encoded by N-PAC, corresponding to a HOST TARGET binding site such as BINDING SITE I, BINDING SITE II or BINDING SITE III. Table 2 illustrates the complementarity of the nucleotide sequences of N-PAC BINDING SITE, designated SEQ ID:2902, to the nucleotide sequence of VGAM55 RNA, herein designated VGAM RNA, also designated SEQ ID:390.

Another function of VGAM55 is therefore inhibition of N-PAC (Accession XM_{—}048113). Accordingly, utilities of VGAM55 include diagnosis, prevention and treatment of diseases and clinical conditions associated with N-PAC. Sema Domain, Immunoglobulin Domain (Ig), Transmembrane Domain (TM) and Short Cytoplasmic Domain, (semaphorin) 4B (SEMA4B, Accession XM_{—}044533) is another VGAM55 host target gene. SEMA4B BINDING SITE is HOST TARGET binding site found in the 5′ untranslated region of mRNA encoded by SEMA4B, corresponding to a HOST TARGET binding site such as BINDING SITE I, BINDING SITE II or BINDING SITE III. Table 2 illustrates the complementarity of the nucleotide sequences of SEMA4B BINDING SITE, designated SEQ ID:2839, to the nucleotide sequence of VGAM55 RNA, herein designated VGAM RNA, also designated SEQ ID:390.

Another function of VGAM55 is therefore inhibition of Sema Domain, Immunoglobulin Domain (Ig), Transmembrane Domain (TM) and Short Cytoplasmic Domain, (semaphorin) 4B (SEMA4B, Accession XM_{—}044533). Accordingly, utilities of VGAM55 include diagnosis, prevention and treatment of diseases and clinical conditions associated with SEMA4B. Sprouty Homolog 1, Antagonist of FGF Signaling (Drosophila) (SPRY1, Accession XM_{—}036349) is another VGAM55 host target gene. SPRY1 BINDING SITE is HOST TARGET binding site found in the 3′ untranslated region of mRNA encoded by SPRY1, corresponding to a HOST TARGET binding site such as BINDING SITE I, BINDING SITE II or BINDING SITE III. Table 2 illustrates the complementarity of the nucleotide sequences of SPRY1 BINDING SITE, designated SEQ ID:2709, to the nucleotide sequence of VGAM55 RNA, herein designated VGAM RNA, also designated SEQ ID:390.

Another function of VGAM55 is therefore inhibition of Sprouty Homolog 1, Antagonist of FGF Signaling (Drosophila) (SPRY1, Accession XM_{—}036349). Accordingly, utilities of VGAM55 include diagnosis, prevention and treatment of diseases and clinical conditions associated with SPRY1. WS-3 (Accession NM_{—}006571) is another VGAM55 host target gene. WS-3 BINDING SITE is HOST TARGET binding site found in the 5′ untranslated region of mRNA encoded by WS-3, corresponding to a HOST TARGET binding site such as BINDING SITE I, BINDING SITE II or BINDING SITE III. Table 2 illustrates the complementarity of the nucleotide sequences of WS-3 BINDING SITE, designated SEQ ID:1312, to the nucleotide sequence of VGAM55 RNA, herein designated VGAM RNA, also designated SEQ ID:390.

Another function of VGAM55 is therefore inhibition of WS-3 (Accession NM_{—}006571). Accordingly, utilities of VGAM55 include diagnosis, prevention and treatment of diseases and clinical conditions associated with WS-3. LOC123242 (Accession XM_{—}063548) is another VGAM55 host target gene. LOC123242 BINDING SITE is HOST TARGET binding site found in the 5′ untranslated region of mRNA encoded by LOC123242, corresponding to a HOST TARGET binding site such as BINDING SITE I, BINDING SITE II or BINDING SITE III. Table 2 illustrates the complementarity of the nucleotide sequences of LOC123242 BINDING SITE, designated SEQ ID:3030, to the nucleotide sequence of VGAM55 RNA, herein designated VGAM RNA, also designated SEQ ID:390.

Another function of VGAM55 is therefore inhibition of LOC123242 (Accession XM_{—}063548). Accordingly, utilities of VGAM55 include diagnosis, prevention and treatment of diseases and clinical conditions associated with LOC123242. LOC127702 (Accession XM_{—}060619) is another VGAM55 host target gene. LOC127702 BINDING SITE is HOST TARGET binding site found in the 3′ untranslated region of mRNA encoded by LOC127702, corresponding to a HOST TARGET binding site such as BINDING SITE I, BINDING SITE II or BINDING SITE III. Table 2 illustrates the complementarity of the nucleotide sequences of LOC127702 BINDING SITE, designated SEQ ID:3024, to the nucleotide sequence of VGAM55 RNA, herein designated VGAM RNA, also designated SEQ ID:390.

Another function of VGAM55 is therefore inhibition of LOC127702 (Accession XM_{—}060619). Accordingly, utilities of VGAM55 include diagnosis, prevention and treatment of diseases and clinical conditions associated with LOC127702. LOC146745 (Accession XM_{—}085577) is another VGAM55 host target gene. LOC146745 BINDING SITE is HOST TARGET binding site found in the 5′ untranslated region of mRNA encoded by LOC146745, corresponding to a HOST TARGET binding site such as BINDING SITE I, BINDING SITE II or BINDING SITE III. Table 2 illustrates the complementarity of the nucleotide sequences of LOC146745 BINDING SITE, designated SEQ ID:3099, to the nucleotide sequence of VGAM55 RNA, herein designated VGAM RNA, also designated SEQ ID:390.

Another function of VGAM55 is therefore inhibition of LOC146745 (Accession XM_{—}085577). Accordingly, utilities of VGAM55 include diagnosis, prevention and treatment of diseases and clinical conditions associated with LOC146745. LOC147791 (Accession XM_{—}097293) is another VGAM55 host target gene. LOC147791 BINDING SITE is HOST TARGET binding site found in the 3′ untranslated region of mRNA encoded by LOC147791, corresponding to a HOST TARGET binding site such as BINDING SITE I, BINDING SITE II or BINDING SITE III. Table 2 illustrates the complementarity of the nucleotide sequences of LOC147791 BINDING SITE, designated SEQ ID:3294, to the nucleotide sequence of VGAM55 RNA, herein designated VGAM RNA, also designated SEQ ID:390.

Another function of VGAM55 is therefore inhibition of LOC147791 (Accession XM_{—}097293). Accordingly, utilities of VGAM55 include diagnosis, prevention and treatment of diseases and clinical conditions associated with LOC147791. LOC196892 (Accession XM_{—}113768) is another VGAM55 host target gene. LOC196892 BINDING SITE is HOST TARGET binding site found in the 3′ untranslated region of mRNA encoded by LOC196892, corresponding to a HOST TARGET binding site such as BINDING SITE I, BINDING SITE II or BINDING SITE III. Table 2 illustrates the complementarity of the nucleotide sequences of LOC196892 BINDING SITE, designated SEQ ID:3417, to the nucleotide sequence of VGAM55 RNA, herein designated VGAM RNA, also designated SEQ ID:390.

Another function of VGAM55 is therefore inhibition of LOC196892 (Accession XM_{—}113768). Accordingly, utilities of VGAM55 include diagnosis, prevention and treatment of diseases and clinical conditions associated with LOC196892. LOC200093 (Accession XM_{—}032184) is another VGAM55 host target gene. LOC200093 BINDING SITE is HOST TARGET binding site found in the 3′ untranslated region of mRNA encoded by LOC200093, corresponding to a HOST TARGET binding site such as BINDING SITE I, BINDING SITE II or BINDING SITE III. Table 2 illustrates the complementarity of the nucleotide sequences of LOC200093 BINDING SITE, designated SEQ ID:2648, to the nucleotide sequence of VGAM55 RNA, herein designated VGAM RNA, also designated SEQ ID:390.

Another function of VGAM55 is therefore inhibition of LOC200093 (Accession XM_{—}032184). Accordingly, utilities of VGAM55 include diagnosis, prevention and treatment of diseases and clinical conditions associated with LOC200093. LOC201243 (Accession XM_{—}113935) is another VGAM55 host target gene. LOC201243 BINDING SITE is HOST TARGET binding site found in the 5′ untranslated region of mRNA encoded by LOC201243, corresponding to a HOST TARGET binding site such as BINDING SITE I, BINDING SITE II or BINDING SITE III. Table 2 illustrates the complementarity of the nucleotide sequences of LOC201243 BINDING SITE, designated SEQ ID:3426, to the nucleotide sequence of VGAM55 RNA, herein designated VGAM RNA, also designated SEQ ID:390.

Another function of VGAM55 is therefore inhibition of LOC201243 (Accession XM_{—}113935). Accordingly, utilities of VGAM55 include diagnosis, prevention and treatment of diseases and clinical conditions associated with LOC201243. LOC219404 (Accession XM_{—}167909) is another VGAM55 host target gene. LOC219404 BINDING SITE is HOST TARGET binding site found in the 3′ untranslated region of mRNA encoded by LOC219404, corresponding to a HOST TARGET binding site such as BINDING SITE I, BINDING SITE II or BINDING SITE III. Table 2 illustrates the complementarity of the nucleotide sequences of LOC219404 BINDING SITE, designated SEQ ID:3606, to the nucleotide sequence of VGAM55 RNA, herein designated VGAM RNA, also designated SEQ ID:390.

Another function of VGAM55 is therefore inhibition of LOC219404 (Accession XM_{—}167909). Accordingly, utilities of VGAM55 include diagnosis, prevention and treatment of diseases and clinical conditions associated with LOC219404. LOC222066 (Accession XM_{—}166582) is another VGAM55 host target gene. LOC222066 BINDING SITE is HOST TARGET binding site found in the 3′ untranslated region of mRNA encoded by LOC222066, corresponding to a HOST TARGET binding site such as BINDING SITE I, BINDING SITE II or BINDING SITE III. Table 2 illustrates the complementarity of the nucleotide sequences of LOC222066 BINDING SITE, designated SEQ ID:3574, to the nucleotide sequence of VGAM55 RNA, herein designated VGAM RNA, also designated SEQ ID:390.

Another function of VGAM55 is therefore inhibition of LOC222066 (Accession XM_{—}166582). Accordingly, utilities of VGAM55 include diagnosis, prevention and treatment of diseases and clinical conditions associated with LOC222066. LOC253001 (Accession XM_{—}171711) is another VGAM55 host target gene. LOC253001 BINDING SITE is HOST TARGET binding site found in the 5′ untranslated region of mRNA encoded by LOC253001, corresponding to a HOST TARGET binding site such as BINDING SITE I, BINDING SITE II or BINDING SITE III. Table 2 illustrates the complementarity of the nucleotide sequences of LOC253001 BINDING SITE, designated SEQ ID:3698, to the nucleotide sequence of VGAM55 RNA, herein designated VGAM RNA, also designated SEQ ID:390.

Another function of VGAM55 is therefore inhibition of LOC253001 (Accession XM_{—}171711). Accordingly, utilities of VGAM55 include diagnosis, prevention and treatment of diseases and clinical conditions associated with LOC253001. LOC91040 (Accession XM_{—}035641) is another VGAM55 host target gene. LOC91040 BINDING SITE is HOST TARGET binding site found in the 3′ untranslated region of mRNA encoded by LOC91040, corresponding to a HOST TARGET binding site such as BINDING SITE I, BINDING SITE II or BINDING SITE III. Table 2 illustrates the complementarity of the nucleotide sequences of LOC91040 BINDING SITE, designated SEQ ID:2700, to the nucleotide sequence of VGAM55 RNA, herein designated VGAM RNA, also designated SEQ ID:390.

Another function of VGAM55 is therefore inhibition of LOC91040 (Accession XM_{—}035641). Accordingly, utilities of VGAM55 include diagnosis, prevention and treatment of diseases and clinical conditions associated with LOC91040. FIG. 1 further provides a conceptual description of a novel bioinformatically detected viral gene of the present invention, referred to here as Viral Genomic Address Messenger 56 (VGAM56) viral gene, which modulates expression of respective host target genes thereof, the function and utility of which host target genes is known in the art.

VGAM56 is a novel bioinformatically detected regulatory, non protein coding, viral micro RNA (miRNA) gene. The method by which VGAM56 was detected is described hereinabove with reference to FIGS. 1-8.

VGAM56 gene, herein designated VGAM GENE, is a viral gene contained in the genome of Vaccinia Virus. VGAM56 host target gene, herein designated VGAM HOST TARGET GENE, is a human gene contained in the human genome.

VGAM56 gene encodes a VGAM56 precursor RNA, herein designated VGAM PRECURSOR RNA. Similar to other miRNA genes, and unlike most ordinary genes, VGAM56 precursor RNA does not encode a protein. A nucleotide sequence identical or highly similar to the nucleotide sequence of VGAM56 precursor RNA is designated SEQ ID:42, and is provided hereinbelow with reference to the sequence listing part. Nucleotide sequence SEQ ID:42 is located at position 183817 relative to the genome of Vaccinia Virus.

VGAM56 precursor RNA folds onto itself, forming VGAM56 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM56 folded precursor RNA into VGAM56 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM56 RNA is designated SEQ ID:391, and is provided hereinbelow with reference to the sequence listing part.

VGAM56 host target gene, herein designated VGAM HOST TARGET GENE, encodes a corresponding messenger RNA, VGAM56 host target RNA, herein designated VGAM HOST TARGET RNA. VGAM56 host target RNA comprises three regions, as is typical of mRNA of a protein coding gene: a untranslated region, a protein coding region and a 3′ untranslated region, designated 5′UTR, PROTEIN CODING and 3′UTR respectively.

VGAM56 RNA, herein designated VGAM RNA, binds complementarily to one or more host target binding sites located in untranslated regions of VGAM56 host target RNA, herein designated VGAM HOST TARGET RNA. This complementary binding is due to the fact that the nucleotide sequence of VGAM56 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM56 RNA, herein designated VGAM RNA, may have a different number of host target binding sites in untranslated regions of a VGAM56 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM56 RNA, herein designated VGAM RNA, to host target binding sites on VGAM56 host target RNA, herein designated VGAM HOST TARGET RNA, such as BINDING SITE I, BINDING SITE II and BINDING SITE III, inhibits translation of VGAM56 host target RNA into VGAM56 host target protein, herein designated VGAM HOST TARGET PROTEIN. VGAM host target protein is therefore outlined by a broken line.

It is appreciated that VGAM56 host target gene, herein designated VGAM HOST TARGET GENE, in fact represents a plurality of VGAM56 host target genes. The mRNA of each one of this plurality of VGAM56 host target genes comprises one or more host target binding sites, each having a nucleotide sequence which is at least partly complementary to VGAM56 RNA, herein designated VGAM RNA, and which when bound by VGAM56 RNA causes inhibition of translation of respective one or more VGAM56 host target proteins.

It is further appreciated by one skilled in the art that the mode of translational inhibition illustrated by FIG. 1 with specific reference to translational inhibition exerted by VGAM56 gene, herein designated VGAM GENE, on one or more VGAM56 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM56 is inhibition of expression of host target genes, as part of a novel viral mechanism of attacking a host. Accordingly, utilities of VGAM56 include diagnosis, prevention and treatment of viral infection by Vaccinia Virus. Specific functions, and accordingly utilities, of VGAM56 correlate with, and may be deduced from, the identity of the host target genes which VGAM56 binds and inhibits, and the function of these host target genes, as elaborated hereinbelow.

Nucleotide sequences of the VGAM56 precursor RNA, herein designated VGAM PRECURSOR RNA, and of the ‘diced’ VGAM56 RNA, herein designated VGAM RNA, and a schematic representation of the secondary folding of VGAM56 folded precursor RNA, herein designated VGAM FOLDED PRECURSOR RNA, of VGAM56 are further described hereinbelow with reference to Table 1.

Nucleotide sequences of host target binding sites, such as BINDING SITE-I, BINDING SITE-II and BINDING SITE-III of FIG. 1, found on VGAM56 host target RNA, and schematic representation of the complementarity of each of these host target binding sites to VGAM56 RNA, herein designated VGAM RNA, are described hereinbelow with reference to Table 2.

As mentioned hereinabove with reference to FIG. 1, a function of VGAM56 gene, herein designated VGAM is inhibition of expression of VGAM56 target genes. It is appreciated that specific functions, and accordingly utilities, of VGAM56 correlate with, and may be deduced from, the identity of the target genes which VGAM56 binds and inhibits, and the function of these target genes, as elaborated hereinbelow.

Glucosaminyl (N-acetyl) Transferase 2,1-branching Enzyme (GCNT2, Accession NM_{—}001491) is a VGAM56 host target gene. GCNT2 BINDING SITE is HOST TARGET binding site found in the 5′ untranslated region of mRNA encoded by GCNT2, corresponding to a HOST TARGET binding site such as BINDING SITE I, BINDING SITE II or BINDING SITE III. Table 2 illustrates the complementarity of the nucleotide sequences of GCNT2 BINDING SITE, designated SEQ ID:831, to the nucleotide sequence of VGAM56 RNA, herein designated VGAM RNA, also designated SEQ ID:391.

A function of VGAM56 is therefore inhibition of Glucosaminyl (N-acetyl) Transferase 2, I-branching Enzyme (GCNT2, Accession NM_{—}001491), a gene which converts linear into branched poly-n-acetyllactosaminoglycans. Accordingly, utilities of VGAM56 include diagnosis, prevention and treatment of diseases and clinical conditions associated with GCNT2. The function of GCNT2 has been established by previous studies. The blood group i/I antigens were the first identified alloantigens that display a dramatic change during human development (OMIM Ref. No. 110800). The i and I antigens are determined by linear and branched poly-N-acetyllactosaminoglycans, respectively. In human erythrocytes during embryonic development, the fetal (i) antigen is replaced by the adult (I) antigen as the result of the appearance of a beta-1,6-N-acetylglucosaminyltransferase, the 1-branching enzyme (GCNT2). Bierhuizen et al. (1993) cloned the cDNA for the branching enzyme that converts the linear form into the branched form and studied its expression with development of I antigen in transfected cells. The cDNA sequence predicted a protein of type II membrane topology as has been found for all other mammalian glycosyltransferases. Comparison of the amino acid sequence with those of other glycosyltransferases revealed that this I-branching enzyme and another beta-1,6, N-acetylglucosaminyltransferase that forms a branch in O-glycans (GCNT1; 600391) are strongly homologous in the center of their putative catalytic domains.

Full details of the abovementioned studies are described in the following publications, the disclosure of which are hereby incorporated by reference:
Bierhuizen, M. F. A.; Mattei, M.-G.; Fukuda, M.: Expression of the developmental I antigen by a cloned human cDNA encoding a member of a beta-1,6-N-acetylglucosaminyltransferase gene family. Genes Dev. 7: 468-478, 1993.; and
Lin-Chu, M.; Broadberry, R. E.; Okubo, Y.; Tanaka, M.: The i phenotype and congenital cataracts among Chinese in Taiwan (Letter) Transfusion 31: 676-677, 1991.

Further studies establishing the function and utilities of GCNT2 are found in John Hopkins OMIM database record ID 600429, and in sited publications numbered 2828-2830, 1044, 172 and 2831-2832 listed in the bibliography section hereinbelow, which are also hereby incorporated by reference. BLP1 (Accession NM_{—}031940) is another VGAM56 host target gene. BLP1 BINDING SITE1 and BLP1 BINDING SITE2 are HOST TARGET binding sites found in untranslated regions of mRNA encoded by BLP1, corresponding to HOST TARGET binding sites such as BINDING SITE I, BINDING SITE II or BINDING SITE III. Table 2 illustrates the complementarity of the nucleotide sequences of BLP1 BINDING SITE1 and BLP1 BINDING SITE2, designated SEQ ID:2222 and SEQ ID:2371 respectively, to the nucleotide sequence of VGAM56 RNA, herein designated VGAM RNA, also designated SEQ ID:391.

Another function of VGAM56 is therefore inhibition of BLP1 (Accession NM_{—}031940). Accordingly, utilities of VGAM56 include diagnosis, prevention and treatment of diseases and clinical conditions associated with BLP1. LOC253891 (Accession XM_{—}170485) is another VGAM56 host target gene. LOC253891 BINDING SITE is HOST TARGET binding site found in the 5′ untranslated region of mRNA encoded by LOC253891, corresponding to a HOST TARGET binding site such as BINDING SITE I, BINDING SITE II or BINDING SITE III. Table 2 illustrates the complementarity of the nucleotide sequences of LOC253891 BINDING SITE, designated SEQ ID:3654, to the nucleotide sequence of VGAM56 RNA, herein designated VGAM RNA, also designated SEQ ID:391.

Another function of VGAM56 is therefore inhibition of LOC253891 (Accession XM_{—}170485). Accordingly, utilities of VGAM56 include diagnosis, prevention and treatment of diseases and clinical conditions associated with LOC253891. FIG. 1 further provides a conceptual description of a novel bioinformatically detected viral gene of the present invention, referred to here as Viral Genomic Address Messenger 57 (VGAM57) viral gene, which modulates expression of respective host target genes thereof, the function and utility of which host target genes is known in the art.

VGAM57 is a novel bioinformatically detected regulatory, non protein coding, viral micro RNA (miRNA) gene. The method by which VGAM57 was detected is described hereinabove with reference to FIGS. 1-8.

VGAM57 gene, herein designated VGAM GENE, is a viral gene contained in the genome of Vaccinia Virus. VGAM57 host target gene, herein designated VGAM HOST TARGET GENE, is a human gene contained in the human genome.

VGAM57 gene encodes a VGAM57 precursor RNA, herein designated VGAM PRECURSOR RNA. Similar to other miRNA genes, and unlike most ordinary genes, VGAM57 precursor RNA does not encode a protein. A nucleotide sequence identical or highly similar to the nucleotide sequence of VGAM57 precursor RNA is designated SEQ ID:43, and is provided hereinbelow with reference to the sequence listing part. Nucleotide sequence SEQ ID:43 is located at position 183735 relative to the genome of Vaccinia Virus.

VGAM57 precursor RNA folds onto itself, forming VGAM57 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM57 folded precursor RNA into VGAM57 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM57 RNA is designated SEQ ID:392, and is provided hereinbelow with reference to the sequence listing part.

VGAM57 host target gene, herein designated VGAM HOST TARGET GENE, encodes a corresponding messenger RNA, VGAM57 host target RNA, herein designated VGAM HOST TARGET RNA. VGAM57 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM57 RNA, herein designated VGAM RNA, binds complementarily to one or more host target binding sites located in untranslated regions of VGAM57 host target RNA, herein designated VGAM HOST TARGET RNA. This complementary binding is due to the fact that the nucleotide sequence of VGAM57 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM57 RNA, herein designated VGAM RNA, may have a different number of host target binding sites in untranslated regions of a VGAM57 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM57 RNA, herein designated VGAM RNA, to host target binding sites on VGAM57 host target RNA, herein designated VGAM HOST TARGET RNA, such as BINDING SITE I, BINDING SITE II and BINDING SITE III, inhibits translation of VGAM57 host target RNA into VGAM57 host target protein, herein designated VGAM HOST TARGET PROTEIN. VGAM host target protein is therefore outlined by a broken line.

It is appreciated that VGAM57 host target gene, herein designated VGAM HOST TARGET GENE, in fact represents a plurality of VGAM57 host target genes. The mRNA of each one of this plurality of VGAM57 host target genes comprises one or more host target binding sites, each having a nucleotide sequence which is at least partly complementary to VGAM57 RNA, herein designated VGAM RNA, and which when bound by VGAM57 RNA causes inhibition of translation of respective one or more VGAM57 host target proteins.

It is further appreciated by one skilled in the art that the mode of translational inhibition illustrated by FIG. 1 with specific reference to translational inhibition exerted by VGAM57 gene, herein designated VGAM GENE, on one or more VGAM57 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM57 is inhibition of expression of host target genes, as part of a novel viral mechanism of attacking a host. Accordingly, utilities of VGAM57 include diagnosis, prevention and treatment of viral infection by Vaccinia Virus. Specific functions, and accordingly utilities, of VGAM57 correlate with, and may be deduced from, the identity of the host target genes which VGAM57 binds and inhibits, and the function of these host target genes, as elaborated hereinbelow.

Nucleotide sequences of the VGAM57 precursor RNA, herein designated VGAM PRECURSOR RNA, and of the ‘diced’ VGAM57 RNA, herein designated VGAM RNA, and a schematic representation of the secondary folding of VGAM57 folded precursor RNA, herein designated VGAM FOLDED PRECURSOR RNA, of VGAM57 are further described hereinbelow with reference to Table 1.

Nucleotide sequences of host target binding sites, such as BINDING SITE-I, BINDING SITE-II and BINDING SITE-III of FIG. 1, found on VGAM57 host target RNA, and schematic representation of the complementarity of each of these host target binding sites to VGAM57 RNA, herein designated VGAM RNA, are described hereinbelow with reference to Table 2.

As mentioned hereinabove with reference to FIG. 1, a function of VGAM57 gene, herein designated VGAM is inhibition of expression of VGAM57 target genes. It is appreciated that specific functions, and accordingly utilities, of VGAM57 correlate with, and may be deduced from, the identity of the target genes which VGAM57 binds and inhibits, and the function of these target genes, as elaborated hereinbelow.

Adenylosuccinate Synthase (ADSS, Accession XM_{—}049992) is a VGAM57 host target gene. ADSS BINDING SITE is HOST TARGET binding site found in the 3′ untranslated region of mRNA encoded by ADSS, corresponding to a HOST TARGET binding site such as BINDING SITE I, BINDING SITE II or BINDING SITE III. Table 2 illustrates the complementarity of the nucleotide sequences of ADSS BINDING SITE, designated SEQ ID:2925, to the nucleotide sequence of VGAM57 RNA, herein designated VGAM RNA, also designated SEQ ID:392.

A function of VGAM57 is therefore inhibition of Adenylosuccinate Synthase (ADSS, Accession XM_{—}049992), a gene which plays an important role in the de novo pathway of purine nucleotide biosynthesis. Accordingly, utilities of VGAM57 include diagnosis, prevention and treatment of diseases and clinical conditions associated with ADSS. The function of ADSS has been established by previous studies. Somatic cell hybrids between human cells and Chinese hamster ovary cells deficient in specific steps in the purine biosynthetic pathway permitted mapping of human genes correcting the defects. The ade(−)H mutant is missing the enzyme adenylosuccinate synthetase (IMP: L-aspartate ligase; EC 6.3.4.4.), which carries out the first of a 2-step sequence in the biosynthesis of AMP from IMP. Thus, ade(−)H cells require exogenous adenine for growth. Lai et al. (1989) found that in somatic cell hybrids human chromosome 1 corrected the defect so that the hybrid cell containing chromosome 1 grew without adenine. Lai et al. (1991) reported that analysis of a human/CHO translocation chromosome that arose in 1 of the hybrids suggested that the gene correcting the defect lies in the region lcen-q12. (See their FIG. 1 for a useful diagram of the purine biosynthesis pathway and the purine nucleotide cycle pathway, together with the location of the genes for the enzymes when known.) AMP deaminase, which converts AMP back to IMP, is coded by a gene, perhaps 2 genes, in region 1p21-p13; see 102770. From a human liver library, Powell et al. (1992) isolated a cDNA that encoded a protein of 455 amino acids. Alignment with the sequence of the ADSS gene in mouse, Dictyostelium discoideum, and E. coli pointed to invariant residues that are likely to be important for structure and/or catalysis. The human ADSS sequence also showed some similarity to argininosuccinate synthetase, which catalyzes a chemically similar reaction.

Full details of the abovementioned studies are described in the following publications, the disclosure of which are hereby incorporated by reference:
Lai, L.-W.; Hart, I. M.; Patterson, D.: A gene correcting the defect in the CHO mutant Ade(−)H, deficient in a branch point enzyme (adenylosuccinate synthetase) of de novo purine biosynthesis, is located on the long arm of chromosome 1. Genomics 9: 322-328, 1991.; and
Powell, S. M.; Zalkin, H.; Dixon, J. E.: Cloning and characterization of the cDNA encoding human adenylosuccinate synthetase. FEBS Lett. 303: 4-10, 1992.

Further studies establishing the function and utilities of ADSS are found in John Hopkins OMIM database record ID 103060, and in sited publications numbered 81-83 listed in the bibliography section hereinbelow, which are also hereby incorporated by reference. Caspase 6, Apoptosis-related Cysteine Protease (CASP6, Accession NM_{—}032992) is another VGAM57 host target gene. CASP6 BINDING SITE1 and CASP6 BINDING SITE2 are HOST TARGET binding sites found in untranslated regions of mRNA encoded by CASP6, corresponding to HOST TARGET binding sites such as BINDING SITE I, BINDING SITE II or BINDING SITE III. Table 2 illustrates the complementarity of the nucleotide sequences of CASP6 BINDING SITE1 and CASP6 BINDING SITE2, designated SEQ ID:2305 and SEQ ID:810 respectively, to the nucleotide sequence of VGAM57 RNA, herein designated VGAM RNA, also designated SEQ ID:392.

Another function of VGAM57 is therefore inhibition of Caspase 6, Apoptosis-related Cysteine Protease (CASP6, Accession NM_{—}032992), a gene which involves in the activation cascade of caspases responsible for apoptosis execution. Accordingly, utilities of VGAM57 include diagnosis, prevention and treatment of diseases and clinical conditions associated with CASP6. The function of CASP6 has been established by previous studies. Fernandes-Alnemri et al. (1995) isolated MCH2, a member of the ced-3 sub family of apoptotic proteases, by performing PCR on human Jurkat T lymphocytes using degenerate oligonucleotides corresponding to conserved peptides in known apoptotic cysteine proteases. The gene, also symbolized CASP6, encodes a 34-kD protein that is highly homologous to human CPP32 (OMIM Ref. No. 600636), C. elegans ced-3, mammalian Ich1/Nedd2 (OMIM Ref. No. 600639), and mammalian interleukin-1-beta converting enzyme (OMIM Ref. No. 147678). Fernandes-Alnemri et al. (1995) observed 1.7-kb (alpha) and 1.4-kb (beta) transcripts expressed in Jurkat lymphocytes and other cell lines. The authors suggested that these transcripts are alternate splicing variants and found that the alpha, but not the beta, MCH2 protein has protease activity. They also found that MCH2-alpha protein can cleave poly(ADP-ribose) polymerase (OMIM Ref. No. 173870) in vitro and that its overexpression induces apoptosis in insect Sf9 cells, suggesting that MCH2 is a mediator of apoptosis in mammalian cells. Using protease assays and immunoblotting experiments, Orth et al. (1996) showed that MCH2, like CPP32 and MCH3, functions downstream of the mammalian cell death inhibitors Bcl2 (OMIM Ref. No. 151430) and BclXL and of the viral serpin CrmA. Further, they found that granzyme B can functionally activate MCH2, supporting the idea that granzyme B kills cells by activating downstream components of the CED-3/ICE apoptotic pathway. Orth et al. (1996) also showed that MCH2, unlike CPP32 and MCH3, can cleave lamin A to its signature apoptotic fragment.

Full details of the abovementioned studies are described in the following publications, the disclosure of which are hereby incorporated by reference:
Fernandes-Alnemri, T.; Litwack, G.; Alnemri, E. S.: Mch2, a new member of the apoptotic Ced-3/Ice cysteine protease gene family. Cancer Res. 55: 2737-2742, 1995.; and
Orth, K.; Chinnaiyan, A. M.; Garg, M.; Froelich, C. J.; Dixit, V. M.: The CED-3/ICE-like protease Mch2 is activated during apoptosis and cleaves the death substrate lamin A. J. Biol. Ch.

Further studies establishing the function and utilities of CASP6 are found in John Hopkins OMIM database record ID 601532, and in sited publications numbered 1637, 163 and 1639 listed in the bibliography section hereinbelow, which are also hereby incorporated by reference. Coagulation Factor II (thrombin) Receptor (F2R, Accession NM_{—}001992) is another VGAM57 host target gene. F2R BINDING SITE is HOST TARGET binding site found in the 3′ untranslated region of mRNA encoded by F2R, corresponding to a HOST TARGET binding site such as BINDING SITE I, BINDING SITE II or BINDING SITE III. Table 2 illustrates the complementarity of the nucleotide sequences of F2R BINDING SITE, designated SEQ ID:879, to the nucleotide sequence of VGAM57 RNA, herein designated VGAM RNA, also designated SEQ ID:392.

Another function of VGAM57 is therefore inhibition of Coagulation Factor II (thrombin) Receptor (F2R, Accession NM_{—}001992), a gene which Thrombin receptor; G protein-coupled receptor involved in platelet activation. Accordingly, utilities of VGAM57 include diagnosis, prevention and treatment of diseases and clinical conditions associated with F2R. The function of F2R has been established by previous studies. Coughlin et al. (1992) reviewed the cloning and characterization of a platelet thrombin (OMIM Ref. No. 176930) receptor (Vu et al., 1991). The thrombin receptor is structurally related to other members of the 7-transmembrane receptor family and has been isolated from diverse cell types. It is intimately involved in the regulation of the thrombotic response. Using PCR analyses of a human/rodent hybrid cell mapping panel, Bahou et al. (1993) assigned the TR gene to chromosome 5. By fluorescence in situ hybridization, they refined the localization to 5q13, confirming its presence as a single locus in the human genome. Poirier et al. (1996) mapped the Cf2r gene to mouse chromosome 13 by studies of an interspecific backcross. Utilizing 2 distinct radiation hybrid mapping panels with different levels of resolution, Schmidt et al. (1997) demonstrated that this gene, sometimes referred to as PAR1, and the proteinase activated receptor-2 gene (OMIM Ref. No. 600933) are tightly linked. Physical mapping using yeast artificial chromosomes and inversion field gel electrophoresis demonstrated that they are maximally separated by 90 kb. Riewald et al. (2002) demonstrated that activated protein C (OMIM Ref. No. 176860) uses the endothelial cell protein C receptor (EPCR; 600646) as a coreceptor for cleavage of protease-activated receptor 1 (PARL) on endothelial cells. Gene profiling demonstrated that PAR1 signaling could account for all activated protein C-induced protective genes, including the immunomodulatory monocyte chemoattractant protein-1 (MCP1; 158105), which was selectively induced by activation of PAR1, but not PAR2 (OMIM Ref. No. 600933). Thus, Riewald et al. (2002) concluded that the prototypical thrombin receptor is the target for EPCR-dependent APC signaling, suggesting a role for this receptor cascade in protection from sepsis. Animal model experiments lend further support to the function of F2R. Griffin et al. (2001) reported a role for Par1, a protease-activated G protein-coupled receptor for thrombin, in embryonic development. Approximately one-half of Par1 −/− embryos died at midgestation with bleeding from multiple sites. Par1 is expressed in endothelial cells, and a Par1 transgene driven by an endothelial-specific promoter prevented death of Par1 −/− embryos. Griffin et al. (2001) concluded that the coagulation cascade and PAR1 modulate endothelial cell function in developing blood vessels and that thrombin's actions on endothelial cells, rather than on platelets, mesenchymal cells, or fibrinogen (see OMIM Ref. No. 134820), contribute to vascular development and hemostasis in the mouse embryo.

It is appreciated that the abovementioned animal model for F2R is acknowledged by those skilled in the art as a scientifically valid animal model, as can be further appreciated from the publications sited hereinbelow.

Full details of the abovementioned studies are described in the following publications, the disclosure of which are hereby incorporated by reference:
Coughlin, S. R.; Vu, T.-K. H.; Hung, D. T.; Wheaton, V. I.: Characterization of a functional thrombin receptor: issues and opportunities. J. Clin. Invest. 89: 351-355, 1992.; and
Riewald, M.; Petrovan, R. J.; Donner, A.; Mueller, B. M.; Ruf, W.: Activation of endothelial cell protease activated receptor 1 by the protein C pathway. Science 296: 1880-1882, 2002.

Further studies establishing the function and utilities of F2R are found in John Hopkins OMIM database record ID 187930, and in sited publications numbered 367-37 and 1330-373 listed in the bibliography section hereinbelow, which are also hereby incorporated by reference. G Protein-coupled Receptor Kinase 7 (GPRK7, Accession NM_{—}139209) is another VGAM57 host target gene. GPRK7 BINDING SITE is HOST TARGET binding site found in the 3′ untranslated region of mRNA encoded by GPRK7, corresponding to a HOST TARGET binding site such as BINDING SITE I, BINDING SITE II or BINDING SITE III. Table 2 illustrates the complementarity of the nucleotide sequences of GPRK7 BINDING SITE, designated SEQ ID:2476, to the nucleotide sequence of VGAM57 RNA, herein designated VGAM RNA, also designated SEQ ID:392.

Another function of VGAM57 is therefore inhibition of G Protein-coupled Receptor Kinase 7 (GPRK7, Accession NM_{—}139209), a gene which regulates the G protein-coupled receptors. Accordingly, utilities of VGAM57 include diagnosis, prevention and treatment of diseases and clinical conditions associated with GPRK7. The function of GPRK7 has been established by previous studies. By PCR on neutrophil cDNA using primers based on sequences of known receptor kinases, Haribabu and Snyderman (1993) identified sequences for GPRK5 (OMIM Ref. No. 600870), GPRK6 (OMIM Ref. No. 600869), and what they believed to be GPRK7. The sequence designated GPRK7 showed the least homology to known receptor kinases and was later determined to match the sequence of mitogen-activated protein kinase-interacting serine/threonine kinase-2 (MKNK2). MKNK2 contains conserved DLG (asp-leu-gly) and ENIL (glu-asn-ile-leu) motifs. Using a 2-hybrid screen for Erk2 (MAPK1; 176948)-binding proteins, Waskiewicz et al. (1997) identified mouse Mnk2 and isolated a full-length cDNA clone from a mouse embryo cDNA library. Mnk2 has a conserved C-terminal Erk-interacting domain, a catalytic domain with homology to the calcium/calmodulin-dependent family of kinases, and putative MAP kinase phosphorylation sites located within the T loop of the kinase domain. Northern blot analysis detected Mnk2 expression in all adult mouse tissues tested except brain, where levels were greatly reduced. Expression was especially abundant in skeletal muscle.

Full details of the abovementioned studies are described in the following publications, the disclosure of which are hereby incorporated by reference:
Haribabu, B.; Snyderman, R.: Identification of additional members of human G-protein-coupled receptor kinase multigene family. Proc. Nat. Acad. Sci. 90: 9398-9402, 1993.; and
Waskiewicz, A. J.; Flynn, A.; Proud, C. G.; Cooper, J. A.: Mitogen-activated protein kinases activate the serine/threonine kinases Mnk1 and Mnk2. EMBO J. 16: 1909-1920, 1997.

Further studies establishing the function and utilities of GPRK7 are found in John Hopkins OMIM database record ID 605069, and in sited publications numbered 1614 listed in the bibliography section hereinbelow, which are also hereby incorporated by reference. Neuroligin 1 (NLGN1, Accession NM_{—}014932) is another VGAM57 host target gene. NLGN1 BINDING SITE is HOST TARGET binding site found in the 3′ untranslated region of mRNA encoded by NLGN1, corresponding to a HOST TARGET binding site such as BINDING SITE I, BINDING SITE II or BINDING SITE III. Table 2 illustrates the complementarity of the nucleotide sequences of NLGN1 BINDING SITE, designated SEQ ID:1591, to the nucleotide sequence of VGAM57 RNA, herein designated VGAM RNA, also designated SEQ ID:392.

Another function of VGAM57 is therefore inhibition of Neuroligin 1 (NLGN1, Accession NM_{—}014932), a gene which may trigger the de novo formation of presynaptic structure. Accordingly, utilities of VGAM57 include diagnosis, prevention and treatment of diseases and clinical conditions associated with NLGN1. The function of NLGN1 has been established by previous studies. Neurexins (see OMIM Ref. No. NRXN1; 600565) are neuronal cell surface proteins first identified in rat brain with hundreds of isoforms generated by alternative splicing. Ichtchenko et al. (1995) described neuroligin I, a neuronal cell surface protein that is enriched in rat synaptic plasma membranes and acts as a splice site-specific ligand for beta-neurexins. (Each of 3 genes encoding neurexins—NRXN1, NRXN2, and NRXN3—has 2 independent promoters and generates 2 classes of mRNAs. The longer mRNAs encode alpha-neurexins and the shorter mRNAs beta-neurexins.) Neuroligin I binds to beta-neurexins only if they lack an insert in the alternatively spliced sequence of the G domain, and not if they contain an insert. The extracellular sequence of rat neuroligin I is composed of a catalytically inactive esterase domain homologous to acetylcholinesterase. Ichtchenko et al. (1995) used in situ hybridization to demonstrate that alternative splicing of neurexins at the binding site recognized by neuroligin I is highly regulated. These findings support a model whereby alternative splicing of neurexins creates a family of cell surface receptors that confer interactive specificity on their resident neurons. Using an in vitro system, Scheiffele et al. (2000) demonstrated that mouse neuroligin-1 and -2, postsynaptically localized proteins, can trigger the de novo formation of presynaptic structure. Normeuronal cells engineered to express neuroligins induced morphologic and functional presynaptic differentiation in contacting axons. This activity could be inhibited by addition of a soluble version of beta-neurexin. Furthermore, addition of soluble beta-neurexin to a coculture of defined pre- and postsynaptic central nervous system (CNS) neurons inhibited synaptic vesicle clustering in axons contacting target neurons. These results suggested that neuroligins are part of the machinery employed during the formation and remodeling of CNS synapses.

Full details of the abovementioned studies are described in the following publications, the disclosure of which are hereby incorporated by reference:
Ichtchenko, K.; Hata, Y.; Nguyen, T.; Ullrich, B.; Missler, M.; Moomaw, C.; Sudhof, T. C.: Neuroligin 1: a splice site-specific ligand for beta-neurexins. Cell 81: 435-443, 1995.; and
Scheiffele, P.; Fan, J.; Choih, J.; Fetter, R.; Serafini, T.: Neuroligin expressed in nonneuronal cells triggers presynaptic development in contacting axons. Cell 101: 657-669, 2000.

Further studies establishing the function and utilities of NLGN1 are found in John Hopkins OMIM database record ID 600568, and in sited publications numbered 1860, 191 and 2150 listed in the bibliography section hereinbelow, which are also hereby incorporated by reference. APC10 (Accession NM_{—}014885) is another VGAM57 host target gene. APC10 BINDING SITE is HOST TARGET binding site found in the 5′ untranslated region of mRNA encoded by APC10, corresponding to a HOST TARGET binding site such as BINDING SITE I, BINDING SITE II or BINDING SITE III. Table 2 illustrates the complementarity of the nucleotide sequences of APC10 BINDING SITE, designated SEQ ID:1574, to the nucleotide sequence of VGAM57 RNA, herein designated VGAM RNA, also designated SEQ ID:392.

Another function of VGAM57 is therefore inhibition of APC10 (Accession NM_{—}014885). Accordingly, utilities of VGAM57 include diagnosis, prevention and treatment of diseases and clinical conditions associated with APC10. Chromosome 20 Open Reading Frame 139 (C20orf139, Accession XM_{—}097749) is another VGAM57 host target gene. C20orf139 BINDING SITE is HOST TARGET binding site found in the 3′ untranslated region of mRNA encoded by C20orf139, corresponding to a HOST TARGET binding site such as BINDING SITE I, BINDING SITE II or BINDING SITE III. Table 2 illustrates the complementarity of the nucleotide sequences of C20orf139 BINDING SITE, designated SEQ ID:3319, to the nucleotide sequence of VGAM57 RNA, herein designated VGAM RNA, also designated SEQ ID:392.

Another function of VGAM57 is therefore inhibition of Chromosome 20 Open Reading Frame 139 (C20orf139, Accession XM_{—}097749). Accordingly, utilities of VGAM57 include diagnosis, prevention and treatment of diseases and clinical conditions associated with C20orf139. FLJ11186 (Accession NM_{—}018353) is another VGAM57 host target gene. FLJ11186 BINDING SITE is HOST TARGET binding site found in the 3′ untranslated region of mRNA encoded by FLJ11186, corresponding to a HOST TARGET binding site such as BINDING SITE I, BINDING SITE II or BINDING SITE III. Table 2 illustrates the complementarity of the nucleotide sequences of FLJ11186 BINDING SITE, designated SEQ ID:1818, to the nucleotide sequence of VGAM57 RNA, herein designated VGAM RNA, also designated SEQ ID:392.

Another function of VGAM57 is therefore inhibition of FLJ11186 (Accession NM_{—}018353). Accordingly, utilities of VGAM57 include diagnosis, prevention and treatment of diseases and clinical conditions associated with FLJ11186. FLJ22116 (Accession NM_{—}024624) is another VGAM57 host target gene. FLJ22116 BINDING SITE is HOST TARGET binding site found in the 5′ untranslated region of mRNA encoded by FLJ22116, corresponding to a HOST TARGET binding site such as BINDING SITE I, BINDING SITE II or BINDING SITE III. Table 2 illustrates the complementarity of the nucleotide sequences of FLJ22116 BINDING SITE, designated SEQ ID:2073, to the nucleotide sequence of VGAM57 RNA, herein designated VGAM RNA, also designated SEQ ID:392.

Another function of VGAM57 is therefore inhibition of FLJ22116 (Accession NM_{—}024624). Accordingly, utilities of VGAM57 include diagnosis, prevention and treatment of diseases and clinical conditions associated with FLJ22116. FLJ32978 (Accession NM_{—}144625) is another VGAM57 host target gene. FLJ32978 BINDING SITE is HOST TARGET binding site found in the 3′ untranslated region of mRNA encoded by FLJ32978, corresponding to a HOST TARGET binding site such as BINDING SITE I, BINDING SITE II or BINDING SITE III. Table 2 illustrates the complementarity of the nucleotide sequences of FLJ32978 BINDING SITE, designated SEQ ID:2487, to the nucleotide sequence of VGAM57 RNA, herein designated VGAM RNA, also designated SEQ ID:392.

Another function of VGAM57 is therefore inhibition of FLJ32978 (Accession NM_{—}144625). Accordingly, utilities of VGAM57 include diagnosis, prevention and treatment of diseases and clinical conditions associated with FLJ32978. KIAA1164 (Accession XM_{—}045358) is another VGAM57 host target gene. KIAA1164 BINDING SITE is HOST TARGET binding site found in the 3′ untranslated region of mRNA encoded by KIAA1164, corresponding to a HOST TARGET binding site such as BINDING SITE I, BINDING SITE II or BINDING SITE III. Table 2 illustrates the complementarity of the nucleotide sequences of KIAA1164 BINDING SITE, designated SEQ ID:2862, to the nucleotide sequence of VGAM57 RNA, herein designated VGAM RNA, also designated SEQ ID:392.

Another function of VGAM57 is therefore inhibition of KIAA1164 (Accession XM_{—}045358). Accordingly, utilities of VGAM57 include diagnosis, prevention and treatment of diseases and clinical conditions associated with KIAA1164. KIAA1762 (Accession XM_{—}033370) is another VGAM57 host target gene. KIAA1762 BINDING SITE is HOST TARGET binding site found in the 3′ untranslated region of mRNA encoded by KIAA1762, corresponding to a HOST TARGET binding site such as BINDING SITE I, BINDING SITE II or BINDING SITE III. Table 2 illustrates the complementarity of the nucleotide sequences of KIAA1762 BINDING SITE, designated SEQ ID:2668, to the nucleotide sequence of VGAM57 RNA, herein designated VGAM RNA, also designated SEQ ID:392.

Another function of VGAM57 is therefore inhibition of KIAA1762 (Accession XM_{—}033370). Accordingly, utilities of VGAM57 include diagnosis, prevention and treatment of diseases and clinical conditions associated with KIAA1762. KIAA1918 (Accession XM_{—}054951) is another VGAM57 host target gene. KIAA1918 BINDING SITE is HOST TARGET binding site found in the 5′ untranslated region of mRNA encoded by KIAA1918, corresponding to a HOST TARGET binding site such as BINDING SITE I, BINDING SITE II or BINDING SITE III. Table 2 illustrates the complementarity of the nucleotide sequences of KIAA1918 BINDING SITE, designated SEQ ID:2969, to the nucleotide sequence of VGAM57 RNA, herein designated VGAM RNA, also designated SEQ ID:392.

Another function of VGAM57 is therefore inhibition of KIAA1918 (Accession XM_{—}054951). Accordingly, utilities of VGAM57 include diagnosis, prevention and treatment of diseases and clinical conditions associated with KIAA1918. POPX1 (Accession NM_{—}014906) is another VGAM57 host target gene. POPX1 BINDING SITE is HOST TARGET binding site found in the 3′ untranslated region of mRNA encoded by POPX1, corresponding to a HOST TARGET binding site such as BINDING SITE I, BINDING SITE II or BINDING SITE III. Table 2 illustrates the complementarity of the nucleotide sequences of POPX1 BINDING SITE, designated SEQ ID:1583, to the nucleotide sequence of VGAM57 RNA, herein designated VGAM RNA, also designated SEQ ID:392.

Another function of VGAM57 is therefore inhibition of POPX1 (Accession NM_{—}014906). Accordingly, utilities of VGAM57 include diagnosis, prevention and treatment of diseases and clinical conditions associated with POPX1. PRO0800 (Accession NM_{—}018592) is another VGAM57 host target gene. PRO0800 BINDING SITE is HOST TARGET binding site found in the 3′ untranslated region of mRNA encoded by PRO0800, corresponding to a HOST TARGET binding site such as BINDING SITE I, BINDING SITE II or BINDING SITE III. Table 2 illustrates the complementarity of the nucleotide sequences of PRO0800 BINDING SITE, designated SEQ ID:1852, to the nucleotide sequence of VGAM57 RNA, herein designated VGAM RNA, also designated SEQ ID:392.

Another function of VGAM57 is therefore inhibition of PRO0800 (Accession NM_{—}018592). Accordingly, utilities of VGAM57 include diagnosis, prevention and treatment of diseases and clinical conditions associated with PRO0800. LOC142779 (Accession XM_{—}084337) is another VGAM57 host target gene. LOC142779 BINDING SITE is HOST TARGET binding site found in the 3′ untranslated region of mRNA encoded by LOC142779, corresponding to a HOST TARGET binding site such as BINDING SITE I, BINDING SITE II or BINDING SITE III. Table 2 illustrates the complementarity of the nucleotide sequences of LOC142779 BINDING SITE, designated SEQ ID:3050, to the nucleotide sequence of VGAM57 RNA, herein designated VGAM RNA, also designated SEQ ID:392.

Another function of VGAM57 is therefore inhibition of LOC142779 (Accession XM_{—}084337). Accordingly, utilities of VGAM57 include diagnosis, prevention and treatment of diseases and clinical conditions associated with LOC142779. LOC143274 (Accession XM_{—}084477) is another VGAM57 host target gene. LOC143274 BINDING SITE is HOST TARGET binding site found in the 5′ untranslated region of mRNA encoded by LOC143274, corresponding to a HOST TARGET binding site such as BINDING SITE I, BINDING SITE II or BINDING SITE III. Table 2 illustrates the complementarity of the nucleotide sequences of LOC143274 BINDING SITE, designated SEQ ID:3053, to the nucleotide sequence of VGAM57 RNA, herein designated VGAM RNA, also designated SEQ ID:392.

Another function of VGAM57 is therefore inhibition of LOC143274 (Accession XM_{—}084477). Accordingly, utilities of VGAM57 include diagnosis, prevention and treatment of diseases and clinical conditions associated with LOC143274. LOC154089 (Accession XM_{—}087846) is another VGAM57 host target gene. LOC154089 BINDING SITE is HOST TARGET binding site found in the 3′ untranslated region of mRNA encoded by LOC154089, corresponding to a HOST TARGET binding site such as BINDING SITE I, BINDING SITE II or BINDING SITE III. Table 2 illustrates the complementarity of the nucleotide sequences of LOC154089 BINDING SITE, designated SEQ ID:3186, to the nucleotide sequence of VGAM57 RNA, herein designated VGAM RNA, also designated SEQ ID:392.

Another function of VGAM57 is therefore inhibition of LOC154089 (Accession XM_{—}087846). Accordingly, utilities of VGAM57 include diagnosis, prevention and treatment of diseases and clinical conditions associated with LOC154089. LOC169679 (Accession XM_{—}108982) is another VGAM57 host target gene. LOC169679 BINDING SITE is HOST TARGET binding site found in the 3′ untranslated region of mRNA encoded by LOC169679, corresponding to a HOST TARGET binding site such as BINDING SITE I, BINDING SITE II or BINDING SITE III. Table 2 illustrates the complementarity of the nucleotide sequences of LOC169679 BINDING SITE, designated SEQ ID:3404, to the nucleotide sequence of VGAM57 RNA, herein designated VGAM RNA, also designated SEQ ID:392.

Another function of VGAM57 is therefore inhibition of LOC169679 (Accession XM_{—}108982). Accordingly, utilities of VGAM57 include diagnosis, prevention and treatment of diseases and clinical conditions associated with LOC169679. LOC202052 (Accession XM_{—}117355) is another VGAM57 host target gene. LOC202052 BINDING SITE is HOST TARGET binding site found in the 5′ untranslated region of mRNA encoded by LOC202052, corresponding to a HOST TARGET binding site such as BINDING SITE I, BINDING SITE II or BINDING SITE III. Table 2 illustrates the complementarity of the nucleotide sequences of LOC202052 BINDING SITE, designated SEQ ID:3484, to the nucleotide sequence of VGAM57 RNA, herein designated VGAM RNA, also designated SEQ ID:392.

Another function of VGAM57 is therefore inhibition of LOC202052 (Accession XM_{—}117355). Accordingly, utilities of VGAM57 include diagnosis, prevention and treatment of diseases and clinical conditions associated with LOC202052. LOC256021 (Accession XM_{—}172884) is another VGAM57 host target gene. LOC256021 BINDING SITE is HOST TARGET binding site found in the 3′ untranslated region of mRNA encoded by LOC256021, corresponding to a HOST TARGET binding site such as BINDING SITE I, BINDING SITE II or BINDING SITE III. Table 2 illustrates the complementarity of the nucleotide sequences of LOC256021 BINDING SITE, designated SEQ ID:3708, to the nucleotide sequence of VGAM57 RNA, herein designated VGAM RNA, also designated SEQ ID:392.

Another function of VGAM57 is therefore inhibition of LOC256021 (Accession XM_{—}172884). Accordingly, utilities of VGAM57 include diagnosis, prevention and treatment of diseases and clinical conditions associated with LOC256021. LOC91650 (Accession XM_{—}039853) is another VGAM57 host target gene. LOC91650 BINDING SITE is HOST TARGET binding site found in the 5′ untranslated region of mRNA encoded by LOC91650, corresponding to a HOST TARGET binding site such as BINDING SITE I, BINDING SITE II or BINDING SITE III. Table 2 illustrates the complementarity of the nucleotide sequences of LOC91650 BINDING SITE, designated SEQ ID:2765, to the nucleotide sequence of VGAM57 RNA, herein designated VGAM RNA, also designated SEQ ID:392.

Another function of VGAM57 is therefore inhibition of LOC91650 (Accession XM_{—}039853). Accordingly, utilities of VGAM57 include diagnosis, prevention and treatment of diseases and clinical conditions associated with LOC91650. LOC91752 (Accession XM_{—}040403) is another VGAM57 host target gene. LOC91752 BINDING SITE is HOST TARGET binding site found in the 5′ untranslated region of mRNA encoded by LOC91752, corresponding to a HOST TARGET binding site such as BINDING SITE I, BINDING SITE II or BINDING SITE III. Table 2 illustrates the complementarity of the nucleotide sequences of LOC91752 BINDING SITE, designated SEQ ID:2777, to the nucleotide sequence of VGAM57 RNA, herein designated VGAM RNA, also designated SEQ ID:392.

Another function of VGAM57 is therefore inhibition of LOC91752 (Accession XM_{—}040403). Accordingly, utilities of VGAM57 include diagnosis, prevention and treatment of diseases and clinical conditions associated with LOC91752. FIG. 1 further provides a conceptual description of a novel bioinformatically detected viral gene of the present invention, referred to here as Viral Genomic Address Messenger 58 (VGAM58) viral gene, which modulates expression of respective host target genes thereof, the function and utility of which host target genes is known in the art.

VGAM58 is a novel bioinformatically detected regulatory, non protein coding, viral micro RNA (miRNA) gene. The method by which VGAM58 was detected is described hereinabove with reference to FIGS. 1-8.

VGAM58 gene, herein designated VGAM GENE, is a viral gene contained in the genome of Vaccinia Virus. VGAM58 host target gene, herein designated VGAM HOST TARGET GENE, is a human gene contained in the human genome.

VGAM58 gene encodes a VGAM58 precursor RNA, herein designated VGAM PRECURSOR RNA. Similar to other miRNA genes, and unlike most ordinary genes, VGAM58 precursor RNA does not encode a protein. A nucleotide sequence identical or highly similar to the nucleotide sequence of VGAM58 precursor RNA is designated SEQ ID:44, and is provided hereinbelow with reference to the sequence listing part. Nucleotide sequence SEQ ID:44 is located at position 9636 relative to the genome of Vaccinia Virus.

VGAM58 precursor RNA folds onto itself, forming VGAM58 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM58 folded precursor RNA into VGAM58 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM58 RNA is designated SEQ ID:393, and is provided hereinbelow with reference to the sequence listing part.

VGAM58 host target gene, herein designated VGAM HOST TARGET GENE, encodes a corresponding messenger RNA, VGAM58 host target RNA, herein designated VGAM HOST TARGET RNA. VGAM58 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM58 RNA, herein designated VGAM RNA, binds complementarily to one or more host target binding sites located in untranslated regions of VGAM58 host target RNA, herein designated VGAM HOST TARGET RNA. This complementary binding is due to the fact that the nucleotide sequence of VGAM58 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM58 RNA, herein designated VGAM RNA, may have a different number of host target binding sites in untranslated regions of a VGAM58 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM58 RNA, herein designated VGAM RNA, to host target binding sites on VGAM58 host target RNA, herein designated VGAM HOST TARGET RNA, such as BINDING SITE I, BINDING SITE II and BINDING SITE III, inhibits translation of VGAM58 host target RNA into VGAM58 host target protein, herein designated VGAM HOST TARGET PROTEIN. VGAM host target protein is therefore outlined by a broken line.

It is appreciated that VGAM58 host target gene, herein designated VGAM HOST TARGET GENE, in fact represents a plurality of VGAM58 host target genes. The mRNA of each one of this plurality of VGAM58 host target genes comprises one or more host target binding sites, each having a nucleotide sequence which is at least partly complementary to VGAM58 RNA, herein designated VGAM RNA, and which when bound by VGAM58 RNA causes inhibition of translation of respective one or more VGAM58 host target proteins.

It is further appreciated by one skilled in the art that the mode of translational inhibition illustrated by FIG. 1 with specific reference to translational inhibition exerted by VGAM58 gene, herein designated VGAM GENE, on one or more VGAM58 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM58 is inhibition of expression of host target genes, as part of a novel viral mechanism of attacking a host. Accordingly, utilities of VGAM58 include diagnosis, prevention and treatment of viral infection by Vaccinia Virus. Specific functions, and accordingly utilities, of VGAM58 correlate with, and may be deduced from, the identity of the host target genes which VGAM58 binds and inhibits, and the function of these host target genes, as elaborated hereinbelow.

Nucleotide sequences of the VGAM58 precursor RNA, herein designated VGAM PRECURSOR RNA, and of the ‘diced’ VGAM58 RNA, herein designated VGAM RNA, and a schematic representation of the secondary folding of VGAM58 folded precursor RNA, herein designated VGAM FOLDED PRECURSOR RNA, of VGAM58 are further described hereinbelow with reference to Table 1.

Nucleotide sequences of host target binding sites, such as BINDING SITE-I, BINDING SITE-II and BINDING SITE-III of FIG. 1, found on VGAM58 host target RNA, and schematic representation of the complementarity of each of these host target binding sites to VGAM58 RNA, herein designated VGAM RNA, are described hereinbelow with reference to Table 2.

As mentioned hereinabove with reference to FIG. 1, a function of VGAM58 gene, herein designated VGAM is inhibition of expression of VGAM58 target genes. It is appreciated that specific functions, and accordingly utilities, of VGAM58 correlate with, and may be deduced from, the identity of the target genes which VGAM58 binds and inhibits, and the function of these target genes, as elaborated hereinbelow.

Dopamine Receptor D1 (DRD1, Accession NM_{—}000794) is a VGAM58 host target gene. DRD1 BINDING SITE is HOST TARGET binding site found in the 3′ untranslated region of mRNA encoded by DRD1, corresponding to a HOST TARGET binding site such as BINDING SITE I, BINDING SITE II or BINDING SITE III. Table 2 illustrates the complementarity of the nucleotide sequences of DRD1 BINDING SITE, designated SEQ ID:774, to the nucleotide sequence of VGAM58 RNA, herein designated VGAM RNA, also designated SEQ ID:393.

A function of VGAM58 is therefore inhibition of Dopamine Receptor D1 (DRD1, Accession NM_{—}000794), a gene which is mediated by g proteins which activate adenylyl cyclase. Accordingly, utilities of VGAM58 include diagnosis, prevention and treatment of diseases and clinical conditions associated with DRD1. The function of DRD1 has been established by previous studies. The diverse physiologic actions of dopamine are mediated by its interaction with 2 types of G protein-coupled receptor, D1 and D2 (OMIM Ref. No. 126450), which stimulate and inhibit, respectively, the enzyme adenylyl cyclase. Three groups reported the cloning of the D1 dopamine receptor gene (Dearry et al., 1990; Zhou et al., 1990; Sunahara et al., 1990). The gene encodes a protein of 446 amino acids having a predicted relative molecular mass of 49,300 and a transmembrane topology similar to that of other G protein-coupled receptors. Northern blot analysis and in situ hybridization showed that the mRNA for this receptor is most abundant in caudate, nucleus accumbens, and olfactory tubercle, with little or no mRNA detectable in substantia nigra, liver, kidney, or heart (Dearry et al., 1990). Animal model experiments lend further support to the function of DRD1. The brain dopaminergic system is a critical modulator of basal ganglion function and plasticity. To investigate the contribution of the dopamine D1 receptor to this modulation, Xu et al. (1994) used gene targeting technology to generate D1 receptor mutant mice. Although histologic analyses suggested no major changes in the anatomy of mutant mouse brains, the expression of dynorphin (OMIM Ref. No. 131340) was greatly reduced in the striatum and related regions of the basal ganglia. The mutant mice did not respond to the stimulant and suppressive effects of D1 receptor agonists and antagonists, respectively, and they exhibited locomotor hyperactivity.

It is appreciated that the abovementioned animal model for DRD1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Dearry, A.; Gingrich, J. A.; Falardeau, P.; Fremeau, R. T., Jr.; Bates, M. D.; Caron, M. G.: Molecular cloning and expression of the gene for a human D(1) dopamine receptor. Nature 347: 72-76, 1990.; and
Xu, M.; Moratalla, R.; Gold, L. H.; Hiroi, N.; Koob, G. F.; Graybiel, A. M.; Tonegawa, S.: Dopamine D1 receptor mutant mice are deficient in striatal expression of dynorphin and in do.

Further studies establishing the function and utilities of DRD1 are found in John Hopkins OMIM database record ID 126449, and in sited publications numbered 2744-2755 listed in the bibliography section hereinbelow, which are also hereby incorporated by reference. KIAA0182 (Accession XM_{—}050495) is another VGAM58 host target gene. KIAA0182 BINDING SITE is HOST TARGET binding site found in the 3′ untranslated region of mRNA encoded by KIAA0182, corresponding to a HOST TARGET binding site such as BINDING SITE I, BINDING SITE II or BINDING SITE III. Table 2 illustrates the complementarity of the nucleotide sequences of KIAA0182 BINDING SITE, designated SEQ ID:2931, to the nucleotide sequence of VGAM58 RNA, herein designated VGAM RNA, also designated SEQ ID:393.

Another function of VGAM58 is therefore inhibition of KIAA0182 (Accession XM_{—}050495). Accordingly, utilities of VGAM58 include diagnosis, prevention and treatment of diseases and clinical conditions associated with KIAA0182. SSH2 (Accession XM_{—}030846) is another VGAM58 host target gene. SSH2 BINDING SITE is HOST TARGET binding site found in the 3′ untranslated region of mRNA encoded by SSH2, corresponding to a HOST TARGET binding site such as BINDING SITE I, BINDING SITE II or BINDING SITE III. Table 2 illustrates the complementarity of the nucleotide sequences of SSH2 BINDING SITE, designated SEQ ID:2626, to the nucleotide sequence of VGAM58 RNA, herein designated VGAM RNA, also designated SEQ ID:393.

Another function of VGAM58 is therefore inhibition of SSH2 (Accession XM_{—}030846). Accordingly, utilities of VGAM58 include diagnosis, prevention and treatment of diseases and clinical conditions associated with SSH2. LOC90918 (Accession XM_{—}034863) is another VGAM58 host target gene. LOC90918 BINDING SITE is HOST TARGET binding site found in the 5′ untranslated region of mRNA encoded by LOC90918, corresponding to a HOST TARGET binding site such as BINDING SITE I, BINDING SITE II or BINDING SITE III. Table 2 illustrates the complementarity of the nucleotide sequences of LOC90918 BINDING SITE, designated SEQ ID:2687, to the nucleotide sequence of VGAM58 RNA, herein designated VGAM RNA, also designated SEQ ID:393.

Another function of VGAM58 is therefore inhibition of LOC90918 (Accession XM_{—}034863). Accordingly, utilities of VGAM58 include diagnosis, prevention and treatment of diseases and clinical conditions associated with LOC90918. FIG. 1 further provides a conceptual description of a novel bioinformatically detected viral gene of the present invention, referred to here as Viral Genomic Address Messenger 59 (VGAM59) viral gene, which modulates expression of respective host target genes thereof, the function and utility of which host target genes is known in the art.

VGAM59 is a novel bioinformatically detected regulatory, non protein coding, viral micro RNA (miRNA) gene. The method by which VGAM59 was detected is described hereinabove with reference to FIGS. 1-8.

VGAM59 gene, herein designated VGAM GENE, is a viral gene contained in the genome of Vaccinia Virus. VGAM59 host target gene, herein designated VGAM HOST TARGET GENE, is a human gene contained in the human genome.

VGAM59 gene encodes a VGAM59 precursor RNA, herein designated VGAM PRECURSOR RNA. Similar to other miRNA genes, and unlike most ordinary genes, VGAM59 precursor RNA does not encode a protein. A nucleotide sequence identical or highly similar to the nucleotide sequence of VGAM59 precursor RNA is designated SEQ ID:45, and is provided hereinbelow with reference to the sequence listing part. Nucleotide sequence SEQ ID:45 is located at position 13536 relative to the genome of Vaccinia Virus.

VGAM59 precursor RNA folds onto itself, forming VGAM59 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM59 folded precursor RNA into VGAM59 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM59 RNA is designated SEQ ID:394, and is provided hereinbelow with reference to the sequence listing part.

VGAM59 host target gene, herein designated VGAM HOST TARGET GENE, encodes a corresponding messenger RNA, VGAM59 host target RNA, herein designated VGAM HOST TARGET RNA. VGAM59 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM59 RNA, herein designated VGAM RNA, binds complementarily to one or more host target binding sites located in untranslated regions of VGAM59 host target RNA, herein designated VGAM HOST TARGET RNA. This complementary binding is due to the fact that the nucleotide sequence of VGAM59 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM59 RNA, herein designated VGAM RNA, may have a different number of host target binding sites in untranslated regions of a VGAM59 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM59 RNA, herein designated VGAM RNA, to host target binding sites on VGAM59 host target RNA, herein designated VGAM HOST TARGET RNA, such as BINDING SITE I, BINDING SITE II and BINDING SITE III, inhibits translation of VGAM59 host target RNA into VGAM59 host target protein, herein designated VGAM HOST TARGET PROTEIN. VGAM host target protein is therefore outlined by a broken line.

It is appreciated that VGAM59 host target gene, herein designated VGAM HOST TARGET GENE, in fact represents a plurality of VGAM59 host target genes. The mRNA of each one of this plurality of VGAM59 host target genes comprises one or more host target binding sites, each having a nucleotide sequence which is at least partly complementary to VGAM59 RNA, herein designated VGAM RNA, and which when bound by VGAM59 RNA causes inhibition of translation of respective one or more VGAM59 host target proteins.

It is further appreciated by one skilled in the art that the mode of translational inhibition illustrated by FIG. 1 with specific reference to translational inhibition exerted by VGAM59 gene, herein designated VGAM GENE, on one or more VGAM59 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM59 is inhibition of expression of host target genes, as part of a novel viral mechanism of attacking a host. Accordingly, utilities of VGAM59 include diagnosis, prevention and treatment of viral infection by Vaccinia Virus. Specific functions, and accordingly utilities, of VGAM59 correlate with, and may be deduced from, the identity of the host target genes which VGAM59 binds and inhibits, and the function of these host target genes, as elaborated hereinbelow.

Nucleotide sequences of the VGAM59 precursor RNA, herein designated VGAM PRECURSOR RNA, and of the ‘diced’ VGAM59 RNA, herein designated VGAM RNA, and a schematic representation of the secondary folding of VGAM59 folded precursor RNA, herein designated VGAM FOLDED PRECURSOR RNA, of VGAM59 are further described hereinbelow with reference to Table 1.

Nucleotide sequences of host target binding sites, such as BINDING SITE-I, BINDING SITE-II and BINDING SITE-III of FIG. 1, found on VGAM59 host target RNA, and schematic representation of the complementarity of each of these host target binding sites to VGAM59 RNA, herein designated VGAM RNA, are described hereinbelow with reference to Table 2.

As mentioned hereinabove with reference to FIG. 1, a function of VGAM59 gene, herein designated VGAM is inhibition of expression of VGAM59 target genes. It is appreciated that specific functions, and accordingly utilities, of VGAM59 correlate with, and may be deduced from, the identity of the target genes which VGAM59 binds and inhibits, and the function of these target genes, as elaborated hereinbelow.

Enoyl-Coenzyme A, Hydratase/3-hydroxyacyl Coenzyme A Dehydrogenase (EHHADH, Accession NM_{—}001966) is a VGAM59 host target gene. EHHADH BINDING SITE is HOST TARGET binding site found in the 3′ untranslated region of mRNA encoded by EHHADH, corresponding to a HOST TARGET binding site such as BINDING SITE I, BINDING SITE II or BINDING SITE III. Table 2 illustrates the complementarity of the nucleotide sequences of EHHADH BINDING SITE, designated SEQ ID:877, to the nucleotide sequence of VGAM59 RNA, herein designated VGAM RNA, also designated SEQ ID:394.

A function of VGAM59 is therefore inhibition of Enoyl-Coenzyme A, Hydratase/3-hydroxyacyl Coenzyme A Dehydrogenase (EHHADH, Accession NM_{—}001966), a gene which functions in the peroxisomal beta-oxidation pathway. Accordingly, utilities of VGAM59 include diagnosis, prevention and treatment of diseases and clinical conditions associated with EHHADH. The function of EHHADH has been established by previous studies. Hoefler et al. (1994) reported the full-length cDNA sequence of the enoyl-CoA-hydratase: 3-hydroxyacyl-CoA dehydrogenase bifunctional enzyme. The cDNA sequence spans 3,779 nucleotides with an open reading frame of 2,169 nucleotides. Animal model experiments lend further support to the function of EHHADH. Qi et al. (1999) generated Lpb null mice. Mutant mice were viable and fertile and exhibited no detectable gross phenotypic defects. The only defect was a blunting of peroxisome proliferative response upon challenge with a peroxisome proliferator. The absence of appreciable changes in lipid metabolism indicated that enoyl-CoAs, generated in the classical system in Lpb null mice, were diverted to the D-hydroxy-specific system for metabolism by Dpb (OMIM Ref. No. 601860).

It is appreciated that the abovementioned animal model for EHHADH is acknowledged by those skilled in the art as a scientifically valid animal model, as can be further appreciated from the publications sited hereinbelow.

Full details of the abovementioned studies are described in the following publications, the disclosure of which are hereby incorporated by reference:
Hoefler, G.; Forstner, M.; McGuinness, M. C.; Hulla, W.; Hiden, M.; Krisper, P.; Kenner, L.; Ried, T.; Lengauer, C.; Zechner, R.; mOser, H. W.; Chen, G. L.: cDNA cloning of the human peroxisomal enoyl-CoA hydratase: 3-hydroxyacyl-CoA dehydrogenase bifunctional enzyme and localization to chromosome 3q26.3-3q28: a free left Alu arm is inserted in the 3-prime noncoding region. Genomics 19: 60-67, 1994.; and
Qi, C.; Zhu, Y.; Pan, J.; Usuda, N.; Maeda, N.; Yeldandi, A. V.; Rao, M. S.; Hashimoto, T.; Reddy, J. K.: Absence of spontaneous peroxisome proliferation in enoyl-CoA hydratase/L-3-hyd.

Further studies establishing the function and utilities of EHHADH are found in John Hopkins OMIM database record ID 607037, and in sited publications numbered 1185 listed in the bibliography section hereinbelow, which are also hereby incorporated by reference. A Disintegrin and Metalloproteinase Domain 9 (meltrin gamma) (ADAM9, Accession NM_{—}003816) is another VGAM59 host target gene. ADAM9 BINDING SITE is HOST TARGET binding site found in the 3′ untranslated region of mRNA encoded by ADAM9, corresponding to a HOST TARGET binding site such as BINDING SITE I, BINDING SITE II or BINDING SITE III. Table 2 illustrates the complementarity of the nucleotide sequences of ADAM9 BINDING SITE, designated SEQ ID:1063, to the nucleotide sequence of VGAM59 RNA, herein designated VGAM RNA, also designated SEQ ID:394.

Another function of VGAM59 is therefore inhibition of A Disintegrin and Metalloproteinase Domain 9 (meltrin gamma) (ADAM9, Accession NM_{—}003816). Accordingly, utilities of VGAM59 include diagnosis, prevention and treatment of diseases and clinical conditions associated with ADAM9. Breast Cancer Anti-estrogen Resistance 3 (BCAR3, Accession NM_{—}003567) is another VGAM59 host target gene. BCAR3 BINDING SITE is HOST TARGET binding site found in the 3′ untranslated region of mRNA encoded by BCAR3, corresponding to a HOST TARGET binding site such as BINDING SITE I, BINDING SITE II or BINDING SITE III. Table 2 illustrates the complementarity of the nucleotide sequences of BCAR3 BINDING SITE, designated SEQ ID:1036, to the nucleotide sequence of VGAM59 RNA, herein designated VGAM RNA, also designated SEQ ID:394.

Another function of VGAM59 is therefore inhibition of Breast Cancer Anti-estrogen Resistance 3 (BCAR3, Accession NM_{—}003567). Accordingly, utilities of VGAM59 include diagnosis, prevention and treatment of diseases and clinical conditions associated with BCAR3. XAP135 (Accession NM_{—}018288) is another VGAM59 host target gene. XAP135 BINDING SITE1 and XAP135 BINDING SITE2 are HOST TARGET binding sites found in untranslated regions of mRNA encoded by XAP135, corresponding to HOST TARGET binding sites such as BINDING SITE I, BINDING SITE II or BINDING SITE III. Table 2 illustrates the complementarity of the nucleotide sequences of XAP135 BINDING SITE1 and XAP135 BINDING SITE2, designated SEQ ID: 1809 and SEQ ID:2408 respectively, to the nucleotide sequence of VGAM59 RNA, herein designated VGAM RNA, also designated SEQ ID:394.

Another function of VGAM59 is therefore inhibition of XAP135 (Accession NM_{—}018288). Accordingly, utilities of VGAM59 include diagnosis, prevention and treatment of diseases and clinical conditions associated with XAP135. FIG. 1 further provides a conceptual description of a novel bioinformatically detected viral gene of the present invention, referred to here as Viral Genomic Address Messenger 60 (VGAM60) viral gene, which modulates expression of respective host target genes thereof, the function and utility of which host target genes is known in the art.

VGAM60 is a novel bioinformatically detected regulatory, non protein coding, viral micro RNA (miRNA) gene. The method by which VGAM60 was detected is described hereinabove with reference to FIGS. 1-8.

VGAM60 gene, herein designated VGAM GENE, is a viral gene contained in the genome of Vaccinia Virus. VGAM60 host target gene, herein designated VGAM HOST TARGET GENE, is a human gene contained in the human genome.

VGAM60 gene encodes a VGAM60 precursor RNA, herein designated VGAM PRECURSOR RNA. Similar to other miRNA genes, and unlike most ordinary genes, VGAM60 precursor RNA does not encode a protein. A nucleotide sequence identical or highly similar to the nucleotide sequence of VGAM60 precursor RNA is designated SEQ ID:46, and is provided hereinbelow with reference to the sequence listing part. Nucleotide sequence SEQ ID:46 is located at position 9389 relative to the genome of Vaccinia Virus.

VGAM60 precursor RNA folds onto itself, forming VGAM60 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM60 folded precursor RNA into VGAM60 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM60 RNA is designated SEQ ID:395, and is provided hereinbelow with reference to the sequence listing part.

VGAM60 host target gene, herein designated VGAM HOST TARGET GENE, encodes a corresponding messenger RNA, VGAM60 host target RNA, herein designated VGAM HOST TARGET RNA. VGAM60 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM60 RNA, herein designated VGAM RNA, binds complementarily to one or more host target binding sites located in untranslated regions of VGAM60 host target RNA, herein designated VGAM HOST TARGET RNA. This complementary binding is due to the fact that the nucleotide sequence of VGAM60 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM60 RNA, herein designated VGAM RNA, may have a different number of host target binding sites in untranslated regions of a VGAM60 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM60 RNA, herein designated VGAM RNA, to host target binding sites on VGAM60 host target RNA, herein designated VGAM HOST TARGET RNA, such as BINDING SITE I, BINDING SITE II and BINDING SITE III, inhibits translation of VGAM60 host target RNA into VGAM60 host target protein, herein designated VGAM HOST TARGET PROTEIN. VGAM host target protein is therefore outlined by a broken line.

It is appreciated that VGAM60 host target gene, herein designated VGAM HOST TARGET GENE, in fact represents a plurality of VGAM60 host target genes. The mRNA of each one of this plurality of VGAM60 host target genes comprises one or more host target binding sites, each having a nucleotide sequence which is at least partly complementary to VGAM60 RNA, herein designated VGAM RNA, and which when bound by VGAM60 RNA causes inhibition of translation of respective one or more VGAM60 host target proteins.

It is further appreciated by one skilled in the art that the mode of translational inhibition illustrated by FIG. 1 with specific reference to translational inhibition exerted by VGAM60 gene, herein designated VGAM GENE, on one or more VGAM60 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM60 is inhibition of expression of host target genes, as part of a novel viral mechanism of attacking a host. Accordingly, utilities of VGAM60 include diagnosis, prevention and treatment of viral infection by Vaccinia Virus. Specific functions, and accordingly utilities, of VGAM60 correlate with, and may be deduced from, the identity of the host target genes which VGAM60 binds and inhibits, and the function of these host target genes, as elaborated hereinbelow.

Nucleotide sequences of the VGAM60 precursor RNA, herein designated VGAM PRECURSOR RNA, and of the ‘diced’ VGAM60 RNA, herein designated VGAM RNA, and a schematic representation of the secondary folding of VGAM60 folded precursor RNA, herein designated VGAM FOLDED PRECURSOR RNA, of VGAM60 are further described hereinbelow with reference to Table 1.

Nucleotide sequences of host target binding sites, such as BINDING SITE-I, BINDING SITE-II and BINDING SITE-III of FIG. 1, found on VGAM60 host target RNA, and schematic representation of the complementarity of each of these host target binding sites to VGAM60 RNA, herein designated VGAM RNA, are described hereinbelow with reference to Table 2.

As mentioned hereinabove with reference to FIG. 1, a function of VGAM60 gene, herein designated VGAM is inhibition of expression of VGAM60 target genes. It is appreciated that specific functions, and accordingly utilities, of VGAM60 correlate with, and may be deduced from, the identity of the target genes which VGAM60 binds and inhibits, and the function of these target genes, as elaborated hereinbelow.

MGC5242 (Accession NM_{—}024033) is a VGAM60 host target gene. MGC5242 BINDING SITE is HOST TARGET binding site found in the 5′ untranslated region of mRNA encoded by MGC5242, corresponding to a HOST TARGET binding site such as BINDING SITE I, BINDING SITE II or BINDING SITE III. Table 2 illustrates the complementarity of the nucleotide sequences of MGC5242 BINDING SITE, designated SEQ ID:2042, to the nucleotide sequence of VGAM60 RNA, herein designated VGAM RNA, also designated SEQ ID:395.

A function of VGAM60 is therefore inhibition of MGC5242 (Accession NM_{—}024033). Accordingly, utilities of VGAM60 include diagnosis, prevention and treatment of diseases and clinical conditions associated with MGC5242. LOC197259 (Accession XM_{—}113849) is another VGAM60 host target gene. LOC197259 BINDING SITE is HOST TARGET binding site found in the 3′ untranslated region of mRNA encoded by LOC197259, corresponding to a HOST TARGET binding site such as BINDING SITE I, BINDING SITE II or BINDING SITE III. Table 2 illustrates the complementarity of the nucleotide sequences of LOC197259 BINDING SITE, designated SEQ ID:3422, to the nucleotide sequence of VGAM60 RNA, herein designated VGAM RNA, also designated SEQ ID:395.

Another function of VGAM60 is therefore inhibition of LOC197259 (Accession XM_{—}113849). Accordingly, utilities of VGAM60 include diagnosis, prevention and treatment of diseases and clinical conditions associated with LOC197259. FIG. 1 further provides a conceptual description of a novel bioinformatically detected viral gene of the present invention, referred to here as Viral Genomic Address Messenger 61 (VGAM61) viral gene, which modulates expression of respective host target genes thereof, the function and utility of which host target genes is known in the art.

VGAM61 is a novel bioinformatically detected regulatory, non protein coding, viral micro RNA (miRNA) gene. The method by which VGAM61 was detected is described hereinabove with reference to FIGS. 1-8.

VGAM61 gene, herein designated VGAM GENE, is a viral gene contained in the genome of Vaccinia Virus. VGAM61 host target gene, herein designated VGAM HOST TARGET GENE, is a human gene contained in the human genome.

VGAM61 gene encodes a VGAM61 precursor RNA, herein designated VGAM PRECURSOR RNA. Similar to other miRNA genes, and unlike most ordinary genes, VGAM61 precursor RNA does not encode a protein. A nucleotide sequence identical or highly similar to the nucleotide sequence of VGAM61 precursor RNA is designated SEQ ID:47, and is provided hereinbelow with reference to the sequence listing part. Nucleotide sequence SEQ ID:47 is located at position 8516 relative to the genome of Vaccinia Virus.

VGAM61 precursor RNA folds onto itself, forming VGAM61 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM61 folded precursor RNA into VGAM61 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM61 RNA is designated SEQ ID:396, and is provided hereinbelow with reference to the sequence listing part.

VGAM61 host target gene, herein designated VGAM HOST TARGET GENE, encodes a corresponding messenger RNA, VGAM61 host target RNA, herein designated VGAM HOST TARGET RNA. VGAM61 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM61 RNA, herein designated VGAM RNA, binds complementarily to one or more host target binding sites located in untranslated regions of VGAM61 host target RNA, herein designated VGAM HOST TARGET RNA. This complementary binding is due to the fact that the nucleotide sequence of VGAM61 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM61 RNA, herein designated VGAM RNA, may have a different number of host target binding sites in untranslated regions of a VGAM61 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM61 RNA, herein designated VGAM RNA, to host target binding sites on VGAM61 host target RNA, herein designated VGAM HOST TARGET RNA, such as BINDING SITE I, BINDING SITE II and BINDING SITE III, inhibits translation of VGAM61 host target RNA into VGAM61 host target protein, herein designated VGAM HOST TARGET PROTEIN. VGAM host target protein is therefore outlined by a broken line.

It is appreciated that VGAM61 host target gene, herein designated VGAM HOST TARGET GENE, in fact represents a plurality of VGAM61 host target genes. The mRNA of each one of this plurality of VGAM61 host target genes comprises one or more host target binding sites, each having a nucleotide sequence which is at least partly complementary to VGAM61 RNA, herein designated VGAM RNA, and which when bound by VGAM61 RNA causes inhibition of translation of respective one or more VGAM61 host target proteins.

It is further appreciated by one skilled in the art that the mode of translational inhibition illustrated by FIG. 1 with specific reference to translational inhibition exerted by VGAM61 gene, herein designated VGAM GENE, on one or more VGAM61 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM61 is inhibition of expression of host target genes, as part of a novel viral mechanism of attacking a host. Accordingly, utilities of VGAM61 include diagnosis, prevention and treatment of viral infection by Vaccinia Virus. Specific functions, and accordingly utilities, of VGAM61 correlate with, and may be deduced from, the identity of the host target genes which VGAM61 binds and inhibits, and the function of these host target genes, as elaborated hereinbelow.

Nucleotide sequences of the VGAM61 precursor RNA, herein designated VGAM PRECURSOR RNA, and of the ‘diced’ VGAM61 RNA, herein designated VGAM RNA, and a schematic representation of the secondary folding of VGAM61 folded precursor RNA, herein designated VGAM FOLDED PRECURSOR RNA, of VGAM61 are further described hereinbelow with reference to Table 1.

Nucleotide sequences of host target binding sites, such as BINDING SITE-I, BINDING SITE-II and BINDING SITE-III of FIG. 1, found on VGAM61 host target RNA, and schematic representation of the complementarity of each of these host target binding sites to VGAM61 RNA, herein designated VGAM RNA, are described hereinbelow with reference to Table 2.

As mentioned hereinabove with reference to FIG. 1, a function of VGAM61 gene, herein designated VGAM is inhibition of expression of VGAM61 target genes. It is appreciated that specific functions, and accordingly utilities, of VGAM61 correlate with, and may be deduced from, the identity of the target genes which VGAM61 binds and inhibits, and the function of these target genes, as elaborated hereinbelow.

DKFZP5641052 (Accession XM_{—}039660) is a VGAM61 host target gene. DKFZP5641052 BINDING SITE is HOST TARGET binding site found in the 3′ untranslated region of mRNA encoded by DKFZP5641052, corresponding to a HOST TARGET binding site such as BINDING SITE I, BINDING SITE II or BINDING SITE III. Table 2 illustrates the complementarity of the nucleotide sequences of DKFZP5641052 BINDING SITE, designated SEQ ID:2759, to the nucleotide sequence of VGAM61 RNA, herein designated VGAM RNA, also designated SEQ ID:396.

A function of VGAM61 is therefore inhibition of DKFZP5641052 (Accession XM_{—}039660). Accordingly, utilities of VGAM61 include diagnosis, prevention and treatment of diseases and clinical conditions associated with DKFZP5641052. KIAA0825 (Accession XM_{—}027906) is another VGAM61 host target gene. KIAA0825 BINDING SITE is HOST TARGET binding site found in the 3′ untranslated region of mRNA encoded by KIAA0825, corresponding to a HOST TARGET binding site such as BINDING SITE I, BINDING SITE II or BINDING SITE III. Table 2 illustrates the complementarity of the nucleotide sequences of KIAA0825 BINDING SITE, designated SEQ ID:2588, to the nucleotide sequence of VGAM61 RNA, herein designated VGAM RNA, also designated SEQ ID:396.

Another function of VGAM61 is therefore inhibition of KIAA0825 (Accession XM_{—}027906). Accordingly, utilities of VGAM61 include diagnosis, prevention and treatment of diseases and clinical conditions associated with KIAA0825. KIAA0976 (Accession NM_{—}014917) is another VGAM61 host target gene. KIAA0976 BINDING SITE is HOST TARGET binding site found in the 3′ untranslated region of mRNA encoded by KIAA0976, corresponding to a HOST TARGET binding site such as BINDING SITE I, BINDING SITE II or BINDING SITE III. Table 2 illustrates the complementarity of the nucleotide sequences of KIAA0976 BINDING SITE, designated SEQ ID:1588, to the nucleotide sequence of VGAM61 RNA, herein designated VGAM RNA, also designated SEQ ID:396.

Another function of VGAM61 is therefore inhibition of KIAA0976 (Accession NM_{—}014917). Accordingly, utilities of VGAM61 include diagnosis, prevention and treatment of diseases and clinical conditions associated with KIAA0976. Placenta-specific 3 (PLAC3, Accession XM_{—}045115) is another VGAM61 host target gene. PLAC3 BINDING SITE is HOST TARGET binding site found in the 3′ untranslated region of mRNA encoded by PLAC3, corresponding to a HOST TARGET binding site such as BINDING SITE I, BINDING SITE II or BINDING SITE III. Table 2 illustrates the complementarity of the nucleotide sequences of PLAC3 BINDING SITE, designated SEQ ID:2853, to the nucleotide sequence of VGAM61 RNA, herein designated VGAM RNA, also designated SEQ ID:396.

Another function of VGAM61 is therefore inhibition of Placenta-specific 3 (PLAC3, Accession XM_{—}045115). Accordingly, utilities of VGAM61 include diagnosis, prevention and treatment of diseases and clinical conditions associated with PLAC3. LOC147991 (Accession XM_{—}085993) is another VGAM61 host target gene. LOC147991 BINDING SITE is HOST TARGET binding site found in the 5′ untranslated region of mRNA encoded by LOC147991, corresponding to a HOST TARGET binding site such as BINDING SITE I, BINDING SITE II or BINDING SITE III. Table 2 illustrates the complementarity of the nucleotide sequences of LOC147991 BINDING SITE, designated SEQ ID:3115, to the nucleotide sequence of VGAM61 RNA, herein designated VGAM RNA, also designated SEQ ID:396.

Another function of VGAM61 is therefore inhibition of LOC147991 (Accession XM_{—}085993). Accordingly, utilities of VGAM61 include diagnosis, prevention and treatment of diseases and clinical conditions associated with LOC147991. FIG. 1 further provides a conceptual description of a novel bioinformatically detected viral gene of the present invention, referred to here as Viral Genomic Address Messenger 62 (VGAM62) viral gene, which modulates expression of respective host target genes thereof, the function and utility of which host target genes is known in the art.

VGAM62 is a novel bioinformatically detected regulatory, non protein coding, viral micro RNA (miRNA) gene. The method by which VGAM62 was detected is described hereinabove with reference to FIGS. 1-8.

VGAM62 gene, herein designated VGAM GENE, is a viral gene contained in the genome of Vaccinia Virus. VGAM62 host target gene, herein designated VGAM HOST TARGET GENE, is a human gene contained in the human genome.

VGAM62 gene encodes a VGAM62 precursor RNA, herein designated VGAM PRECURSOR RNA. Similar to other miRNA genes, and unlike most ordinary genes, VGAM62 precursor RNA does not encode a protein. A nucleotide sequence identical or highly similar to the nucleotide sequence of VGAM62 precursor RNA is designated SEQ ID:48, and is provided hereinbelow with reference to the sequence listing part. Nucleotide sequence SEQ ID:48 is located at position 10036 relative to the genome of Vaccinia Virus.

VGAM62 precursor RNA folds onto itself, forming VGAM62 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM62 folded precursor RNA into VGAM62 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM62 RNA is designated SEQ ID:397, and is provided hereinbelow with reference to the sequence listing part.

VGAM62 host target gene, herein designated VGAM HOST TARGET GENE, encodes a corresponding messenger RNA, VGAM62 host target RNA, herein designated VGAM HOST TARGET RNA. VGAM62 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM62 RNA, herein designated VGAM RNA, binds complementarily to one or more host target binding sites located in untranslated regions of VGAM62 host target RNA, herein designated VGAM HOST TARGET RNA. This complementary binding is due to the fact that the nucleotide sequence of VGAM62 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM62 RNA, herein designated VGAM RNA, may have a different number of host target binding sites in untranslated regions of a VGAM62 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM62 RNA, herein designated VGAM RNA, to host target binding sites on VGAM62 host target RNA, herein designated VGAM HOST TARGET RNA, such as BINDING SITE I, BINDING SITE II and BINDING SITE III, inhibits translation of VGAM62 host target RNA into VGAM62 host target protein, herein designated VGAM HOST TARGET PROTEIN. VGAM host target protein is therefore outlined by a broken line.

It is appreciated that VGAM62 host target gene, herein designated VGAM HOST TARGET GENE, in fact represents a plurality of VGAM62 host target genes. The mRNA of each one of this plurality of VGAM62 host target genes comprises one or more host target binding sites, each having a nucleotide sequence which is at least partly complementary to VGAM62 RNA, herein designated VGAM RNA, and which when bound by VGAM62 RNA causes inhibition of translation of respective one or more VGAM62 host target proteins.

It is further appreciated by one skilled in the art that the mode of translational inhibition illustrated by FIG. 1 with specific reference to translational inhibition exerted by VGAM62 gene, herein designated VGAM GENE, on one or more VGAM62 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM62 is inhibition of expression of host target genes, as part of a novel viral mechanism of attacking a host. Accordingly, utilities of VGAM62 include diagnosis, prevention and treatment of viral infection by Vaccinia Virus. Specific functions, and accordingly utilities, of VGAM62 correlate with, and may be deduced from, the identity of the host target genes which VGAM62 binds and inhibits, and the function of these host target genes, as elaborated hereinbelow.

Nucleotide sequences of the VGAM62 precursor RNA, herein designated VGAM PRECURSOR RNA, and of the ‘diced’ VGAM62 RNA, herein designated VGAM RNA, and a schematic representation of the secondary folding of VGAM62 folded precursor RNA, herein designated VGAM FOLDED PRECURSOR RNA, of VGAM62 are further described hereinbelow with reference to Table 1.

Nucleotide sequences of host target binding sites, such as BINDING SITE-I, BINDING SITE-II and BINDING SITE-III of FIG. 1, found on VGAM62 host target RNA, and schematic representation of the complementarity of each of these host target binding sites to VGAM62 RNA, herein designated VGAM RNA, are described hereinbelow with reference to Table 2.

As mentioned hereinabove with reference to FIG. 1, a function of VGAM62 gene, herein designated VGAM is inhibition of expression of VGAM62 target genes. It is appreciated that specific functions, and accordingly utilities, of VGAM62 correlate with, and may be deduced from, the identity of the target genes which VGAM62 binds and inhibits, and the function of these target genes, as elaborated hereinbelow.

Histone Deacetylase 7A (HDAC7A, Accession NM_{—}015401) is a VGAM62 host target gene. HDAC7A BINDING SITE1 and HDAC7A BINDING SITE2 are HOST TARGET binding sites found in untranslated regions of mRNA encoded by HDAC7A, corresponding to HOST TARGET binding sites such as BINDING SITE I, BINDING SITE II or BINDING SITE III. Table 2 illustrates the complementarity of the nucleotide sequences of HDAC7A BINDING SITE1 and HDAC7A BINDING SITE2, designated SEQ ID: 1628 and SEQ ID: 1700 respectively, to the nucleotide sequence of VGAM62 RNA, herein designated VGAM RNA, also designated SEQ ID:397.

A function of VGAM62 is therefore inhibition of Histone Deacetylase 7A (HDAC7A, Accession NM_{—}015401). Accordingly, utilities of VGAM62 include diagnosis, prevention and treatment of diseases and clinical conditions associated with HDAC7A. Platelet-derived Growth Factor Beta Polypeptide (simian sarcoma viral (v-sis) Oncogene Homolog) (PDGFB, Accession NM_{—}002608) is another VGAM62 host target gene. PDGFB BINDING SITE is HOST TARGET binding site found in the 3′ untranslated region of mRNA encoded by PDGFB, corresponding to a HOST TARGET binding site such as BINDING SITE I, BINDING SITE II or BINDING SITE III. Table 2 illustrates the complementarity of the nucleotide sequences of PDGFB BINDING SITE, designated SEQ ID:941, to the nucleotide sequence of VGAM62 RNA, herein designated VGAM RNA, also designated SEQ ID:397.

Another function of VGAM62 is therefore inhibition of Platelet-derived Growth Factor Beta Polypeptide (simian sarcoma viral (v-sis) Oncogene Homolog) (PDGFB, Accession NM_{—}002608), a gene which plays an important role in stimulating adjacent cells to grow and thereby heal the wound. Accordingly, utilities of VGAM62 include diagnosis, prevention and treatment of diseases and clinical conditions associated with PDGFB. The function of PDGFB has been established by previous studies. Most proliferating cells are programmed to undergo apoptosis unless specific survival signals are provided. Platelet-derived growth factor promotes cellular proliferation and inhibits apoptosis. Romashkova and Makarov (1999) showed that PDGF activates the RAS/PIK3/AKT1/IKK/NFKB1 pathway. In this pathway, NFKB1 (OMIM Ref. No. 164011) does not induce c-myc and apoptosis, but instead induces putative antiapoptotic genes. In response to PDGF, AKT1 (OMIM Ref. No. 164730) transiently associates with IKK (see OMIM Ref. No. 600664) and induces IKK activation. The authors suggested that under certain conditions PIK3 (see OMIM Ref. No. 171834) may activate NFKB1 without the involvement of NFKBIA (OMIM Ref. No. 164008) or NFKBIB (OMIM Ref. No. 604495) degradation. Dermatofibrosarcoma protuberans (DFSP), an infiltrative skin tumor of intermediate malignancy, presents specific cytogenetic features such as reciprocal translocations t(17;22)(q22;q13) and supernumerary ring chromosomes derived from t(17;22). Simon et al. (1997) characterized the breakpoints from translocations and rings in dermatofibrosarcoma protuberans and its juvenile form, giant cell fibroblastoma, on the genomic and RNA levels. They found that these rearrangements fuse the PDGFB gene and the COL1A1 gene (OMIM Ref. No. 120150). Simon et al. (1997) commented that PDGFB has transforming activity and is a potent mitogen for a number of cell types, but its role in oncogenic processes was not fully understood. They noted that neither COL1A1 nor PDGFB had hitherto been implicated in tumor translocations. The gene fusions deleted exon 1 of PDGFB and released this growth factor from its normal regulation; see 190040.0002.

Full details of the abovementioned studies are described in the following publications, the disclosure of which are hereby incorporated by reference:
Simon, M.-P.; Pedeutour, F.; Sirvent, N.; Grosgeorge, J.; Minoletti, F.; Coindre, J.-M.; Terrier-Lacombe, M.-J.; Mandahl, N.; Craver, R. D.; Blin, N.; Sozzi, G.; Turc-Carel, C.; O'Brien, K. P.; Kedra, D.; Fransson, I.; Guilbaud, C.; Dumanski, J. P.: Deregulation of the platelet-derived growth factor B-chain gene via fusion with collagen gene COL1A1 in dermatofibrosarcoma protuberans and giant-cell fibroblastoma. Nature Genet. 15: 95-98, 1997.; and
Josephs, S. F.; Guo, C.; Ratner, L.; Wong-Staal, F.: Human proto-oncogene nucleotide sequences corresponding to the transforming region of simian sarcoma virus. Science 223: 487-491, 1.

Further studies establishing the function and utilities of PDGFB are found in John Hopkins OMIM database record ID 190040, and in sited publications numbered 2377-2387, 2187-2190, 2286-2193, 755-757, 2194-2196, 832, 2197-2201, 3059, 2207, 2660, 220 and 2376 listed in the bibliography section hereinbelow, which are also hereby incorporated by reference. PR Domain Containing 4 (PRDM4, Accession NM_{—}012406) is another VGAM62 host target gene. PRDM4 BINDING SITE is HOST TARGET binding site found in the 3′ untranslated region of mRNA encoded by PRDM4, corresponding to a HOST TARGET binding site such as BINDING SITE I, BINDING SITE II or BINDING SITE III. Table 2 illustrates the complementarity of the nucleotide sequences of PRDM4 BINDING SITE, designated SEQ ID: 1427, to the nucleotide sequence of VGAM62 RNA, herein designated VGAM RNA, also designated SEQ ID:397.

Another function of VGAM62 is therefore inhibition of PR Domain Containing 4 (PRDM4, Accession NM_{—}012406). Accordingly, utilities of VGAM62 include diagnosis, prevention and treatment of diseases and clinical conditions associated with PRDM4. Sirtuin Silent Mating Type Information Regulation 2 Homolog 6 (S. cerevisiae) (SIRT6, Accession NM_{—}016539) is another VGAM62 host target gene. SIRT6 BINDING SITE is HOST TARGET binding site found in the 3′ untranslated region of mRNA encoded by SIRT6, corresponding to a HOST TARGET binding site such as BINDING SITE I, BINDING SITE II or BINDING SITE III. Table 2 illustrates the complementarity of the nucleotide sequences of SIRT6 BINDING SITE, designated SEQ ID:1695, to the nucleotide sequence of VGAM62 RNA, herein designated VGAM RNA, also designated SEQ ID:397.

Another function of VGAM62 is therefore inhibition of Sirtuin Silent Mating Type Information Regulation 2 Homolog 6 (S. cerevisiae) (SIRT6, Accession NM_{—}016539). Accordingly, utilities of VGAM62 include diagnosis, prevention and treatment of diseases and clinical conditions associated with SIRT6. Xylulokinase Homolog (H. influenzae) (XYLB, Accession NM_{—}005108) is another VGAM62 host target gene. XYLB BINDING SITE is HOST TARGET binding site found in the 3′ untranslated region of mRNA encoded by XYLB, corresponding to a HOST TARGET binding site such as BINDING SITE I, BINDING SITE II or BINDING SITE III. Table 2 illustrates the complementarity of the nucleotide sequences of XYLB BINDING SITE, designated SEQ ID:1187, to the nucleotide sequence of VGAM62 RNA, herein designated VGAM RNA, also designated SEQ ID:397.

Another function of VGAM62 is therefore inhibition of Xylulokinase Homolog (H. influenzae) (XYLB, Accession NM_{—}005108), a gene which is similar to Haemophilus influenzae xylulokinase and may play a role in energy metabolism. Accordingly, utilities of VGAM62 include diagnosis, prevention and treatment of diseases and clinical conditions associated with XYLB. The function of XYLB has been established by previous studies. The 3p22-p21.3 chromosomal region is one of 3 regions of 3p that is commonly deleted in various carcinomas. By analysis of a cloned segment from this region, Tamari et al. (1998) identified a novel gene, which they designated XYLB because the predicted 528-amino acid protein shares 22% identity with Hemophilus influenzae xylulokinase (Xyl). The XYLB gene contains 18 exons and spans approximately 28 kb. Northern blot analysis revealed that XYLB was expressed as a 2.3-kb major transcript in all tissues tested. A less abundant 1.8-kb mRNA was detected in heart and skeletal muscle. Daigo et al. (1999) reported that the XYLB gene is located between the OCTL2 (OMIM Ref. No. 604048) and ActRIIB (OMIM Ref. No. 602730) genes on 3p22-p21.3.

Full details of the abovementioned studies are described in the following publications, the disclosure of which are hereby incorporated by reference:
Daigo, Y.; Isomura, M.; Nishiwaki, T.; Tamari, M.; Ishikawa, S.; Kai, M.; Murata, Y.; Takeuchi, K.; Yamane, Y.; Hayashi, R.; Minami, M.; Fujino, M. A.; Hojo, Y.; Uchiyama, I.; Takagi, T.; Nakamura, Y.: Characterization of a 1200-kb genomic segment of chromosome 3p22-p21.3. DNA Res. 6: 37-44, 1999.; and
Tamari, M.; Daigo, Y.; Ishikawa, S.; Nakamura, Y.: Genomic structure of a novel human gene (XYLB) on chromosome 3p22-p21.3 encoding a xylulokinase-like protein. Cytogenet. Cell Genet.

Further studies establishing the function and utilities of XYLB are found in John Hopkins OMIM database record ID 604049, and in sited publications numbered 2027 listed in the bibliography section hereinbelow, which are also hereby incorporated by reference. DKFZP434N093 (Accession XM_{—}086948) is another VGAM62 host target gene. DKFZP434N093 BINDING SITE is HOST TARGET binding site found in the 3′ untranslated region of mRNA encoded by DKFZP434N093, corresponding to a HOST TARGET binding site such as BINDING SITE I, BINDING SITE II or BINDING SITE III. Table 2 illustrates the complementarity of the nucleotide sequences of DKFZP434N093 BINDING SITE, designated SEQ ID:3147, to the nucleotide sequence of VGAM62 RNA, herein designated VGAM RNA, also designated SEQ ID:397.

Another function of VGAM62 is therefore inhibition of DKFZP434N093 (Accession XM_{—}086948). Accordingly, utilities of VGAM62 include diagnosis, prevention and treatment of diseases and clinical conditions associated with DKFZP434N093. DKFZP547E1010 (Accession XM_{—}040002) is another VGAM62 host target gene. DKFZP547E1010 BINDING SITE1 and DKFZP547E1010 BINDING SITE2 are HOST TARGET binding sites found in untranslated regions of mRNA encoded by DKFZP547E1010, corresponding to HOST TARGET binding sites such as BINDING SITE I, BINDING SITE II or BINDING SITE III. Table 2 illustrates the complementarity of the nucleotide sequences of DKFZP547E1010 BINDING SITE1 and DKFZP547E1010 BINDING SITE2, designated SEQ ID:2768 and SEQ ID: 1640 respectively, to the nucleotide sequence of VGAM62 RNA, herein designated VGAM RNA, also designated SEQ ID:397.

Another function of VGAM62 is therefore inhibition of DKFZP547E1010 (Accession XM_{—}040002). Accordingly, utilities of VGAM62 include diagnosis, prevention and treatment of diseases and clinical conditions associated with DKFZP547E1010. FLJ14596 (Accession NM_{—}032809) is another VGAM62 host target gene. FLJ14596 BINDING SITE is HOST TARGET binding site found in the 3′ untranslated region of mRNA encoded by FLJ14596, corresponding to a HOST TARGET binding site such as BINDING SITE I, BINDING SITE II or BINDING SITE III. Table 2 illustrates the complementarity of the nucleotide sequences of FLJ14596 BINDING SITE, designated SEQ ID:2282, to the nucleotide sequence of VGAM62 RNA, herein designated VGAM RNA, also designated SEQ ID:397.

Another function of VGAM62 is therefore inhibition of FLJ14596 (Accession NM_{—}032809). Accordingly, utilities of VGAM62 include diagnosis, prevention and treatment of diseases and clinical conditions associated with FLJ14596. HRLP5 (Accession NM_{—}054108) is another VGAM62 host target gene. HRLP5 BINDING SITE is HOST TARGET binding site found in the 3′ untranslated region of mRNA encoded by HRLP5, corresponding to a HOST TARGET binding site such as BINDING SITE I, BINDING SITE II or BINDING SITE III. Table 2 illustrates the complementarity of the nucleotide sequences of HRLP5 BINDING SITE, designated SEQ ID:2364, to the nucleotide sequence of VGAM62 RNA, herein designated VGAM RNA, also designated SEQ ID:397.

Another function of VGAM62 is therefore inhibition of HRLP5 (Accession NM_{—}054108). Accordingly, utilities of VGAM62 include diagnosis, prevention and treatment of diseases and clinical conditions associated with HRLP5. Potassium Voltage-gated Channel, Delayed-rectifier, Sub family S, Member 1 (KCNS1, Accession NM_{—}002251) is another VGAM62 host target gene. KCNS1 BINDING SITE is HOST TARGET binding site found in the 3′ untranslated region of mRNA encoded by KCNS1, corresponding to a HOST TARGET binding site such as BINDING SITE I, BINDING SITE II or BINDING SITE III. Table 2 illustrates the complementarity of the nucleotide sequences of KCNS1 BINDING SITE, designated SEQ ID:917, to the nucleotide sequence of VGAM62 RNA, herein designated VGAM RNA, also designated SEQ ID:397.

Another function of VGAM62 is therefore inhibition of Potassium Voltage-gated Channel, Delayed-rectifier, Sub family S, Member 1 (KCNS1, Accession NM_{—}002251). Accordingly, utilities of VGAM62 include diagnosis, prevention and treatment of diseases and clinical conditions associated with KCNS1. KIAA0992 (Accession NM_{—}016081) is another VGAM62 host target gene. KIAA0992 BINDING SITE is HOST TARGET binding site found in the 5′ untranslated region of mRNA encoded by KIAA0992, corresponding to a HOST TARGET binding site such as BINDING SITE I, BINDING SITE II or BINDING SITE III. Table 2 illustrates the complementarity of the nucleotide sequences of KIAA0992 BINDING SITE, designated SEQ ID: 1662, to the nucleotide sequence of VGAM62 RNA, herein designated VGAM RNA, also designated SEQ ID:397.

Another function of VGAM62 is therefore inhibition of KIAA0992 (Accession NM_{—}016081). Accordingly, utilities of VGAM62 include diagnosis, prevention and treatment of diseases and clinical conditions associated with KIAA0992. KIAA1975 (Accession XM_{—}058393) is another VGAM62 host target gene. KIAA1975 BINDING SITE is HOST TARGET binding site found in the 5′ untranslated region of mRNA encoded by KIAA1975, corresponding to a HOST TARGET binding site such as BINDING SITE I, BINDING SITE II or BINDING SITE III. Table 2 illustrates the complementarity of the nucleotide sequences of KIAA1975 BINDING SITE, designated SEQ ID:2982, to the nucleotide sequence of VGAM62 RNA, herein designated VGAM RNA, also designated SEQ ID:397.

Another function of VGAM62 is therefore inhibition of KIAA1975 (Accession XM_{—}058393). Accordingly, utilities of VGAM62 include diagnosis, prevention and treatment of diseases and clinical conditions associated with KIAA1975. MGC5590 (Accession NM_{—}024058) is another VGAM62 host target gene. MGC5590 BINDING SITE is HOST TARGET binding site found in the 3′ untranslated region of mRNA encoded by MGC5590, corresponding to a HOST TARGET binding site such as BINDING SITE I, BINDING SITE II or BINDING SITE III. Table 2 illustrates the complementarity of the nucleotide sequences of MGC5590 BINDING SITE, designated SEQ ID:2049, to the nucleotide sequence of VGAM62 RNA, herein designated VGAM RNA, also designated SEQ ID:397.

Another function of VGAM62 is therefore inhibition of MGC5590 (Accession NM_{—}024058). Accordingly, utilities of VGAM62 include diagnosis, prevention and treatment of diseases and clinical conditions associated with MGC5590. Sialyltransferase 8C (alpha2,3Galbeta1,4GlcNAcalpha 2,8-sialyltransferase) (SIAT8C, Accession NM_{—}015879) is another VGAM62 host target gene. SIAT8C BINDING SITE is HOST TARGET binding site found in the 5′ untranslated region of mRNA encoded by SIAT8C, corresponding to a HOST TARGET binding site such as BINDING SITE I, BINDING SITE II or BINDING SITE III. Table 2 illustrates the complementarity of the nucleotide sequences of SIAT8C BINDING SITE, designated SEQ ID: 1648, to the nucleotide sequence of VGAM62 RNA, herein designated VGAM RNA, also designated SEQ ID:397.

Another function of VGAM62 is therefore inhibition of Sialyltransferase 8C (alpha2,3Galbeta1,4GlcNAcalpha 2,8-sialyltransferase) (SIAT8C, Accession NM_{—}015879). Accordingly, utilities of VGAM62 include diagnosis, prevention and treatment of diseases and clinical conditions associated with SIAT8C. LOC149706 (Accession XM_{—}097718) is another VGAM62 host target gene. LOC149706 BINDING SITE is HOST TARGET binding site found in the 3′ untranslated region of mRNA encoded by LOC149706, corresponding to a HOST TARGET binding site such as BINDING SITE I, BINDING SITE II or BINDING SITE III. Table 2 illustrates the complementarity of the nucleotide sequences of LOC149706 BINDING SITE, designated SEQ ID:3313, to the nucleotide sequence of VGAM62 RNA, herein designated VGAM RNA, also designated SEQ ID:397.

Another function of VGAM62 is therefore inhibition of LOC149706 (Accession XM_{—}097718). Accordingly, utilities of VGAM62 include diagnosis, prevention and treatment of diseases and clinical conditions associated with LOC149706. LOC160292 (Accession XM_{—}100817) is another VGAM62 host target gene. LOC160292 BINDING SITE is HOST TARGET binding site found in the 3′ untranslated region of mRNA encoded by LOC160292, corresponding to a HOST TARGET binding site such as BINDING SITE I, BINDING SITE II or BINDING SITE III. Table 2 illustrates the complementarity of the nucleotide sequences of LOC160292 BINDING SITE, designated SEQ ID:3399, to the nucleotide sequence of VGAM62 RNA, herein designated VGAM RNA, also designated SEQ ID:397.

Another function of VGAM62 is therefore inhibition of LOC160292 (Accession XM_{—}100817). Accordingly, utilities of VGAM62 include diagnosis, prevention and treatment of diseases and clinical conditions associated with LOC160292. LOC203378 (Accession XM_{—}117541) is another VGAM62 host target gene. LOC203378 BINDING SITE is HOST TARGET binding site found in the 3′ untranslated region of mRNA encoded by LOC203378, corresponding to a HOST TARGET binding site such as BINDING SITE I, BINDING SITE II or BINDING SITE III. Table 2 illustrates the complementarity of the nucleotide sequences of LOC203378 BINDING SITE, designated SEQ ID:3491, to the nucleotide sequence of VGAM62 RNA, herein designated VGAM RNA, also designated SEQ ID:397.

Another function of VGAM62 is therefore inhibition of LOC203378 (Accession XM_{—}117541). Accordingly, utilities of VGAM62 include diagnosis, prevention and treatment of diseases and clinical conditions associated with LOC203378. LOC204200 (Accession XM_{—}118520) is another VGAM62 host target gene. LOC204200 BINDING SITE is HOST TARGET binding site found in the 5′ untranslated region of mRNA encoded by LOC204200, corresponding to a HOST TARGET binding site such as BINDING SITE I, BINDING SITE II or BINDING SITE III. Table 2 illustrates the complementarity of the nucleotide sequences of LOC204200 BINDING SITE, designated SEQ ID:3493, to the nucleotide sequence of VGAM62 RNA, herein designated VGAM RNA, also designated SEQ ID:397.

Another function of VGAM62 is therefore inhibition of LOC204200 (Accession XM_{—}118520). Accordingly, utilities of VGAM62 include diagnosis, prevention and treatment of diseases and clinical conditions associated with LOC204200. LOC255265 (Accession XM_{—}170902) is another VGAM62 host target gene. LOC255265 BINDING SITE is HOST TARGET binding site found in the 5′ untranslated region of mRNA encoded by LOC255265, corresponding to a HOST TARGET binding site such as BINDING SITE I, BINDING SITE II or BINDING SITE III. Table 2 illustrates the complementarity of the nucleotide sequences of LOC255265 BINDING SITE, designated SEQ ID:3672, to the nucleotide sequence of VGAM62 RNA, herein designated VGAM RNA, also designated SEQ ID:397.

Another function of VGAM62 is therefore inhibition of LOC255265 (Accession XM_{—}170902). Accordingly, utilities of VGAM62 include diagnosis, prevention and treatment of diseases and clinical conditions associated with LOC255265. FIG. 1 further provides a conceptual description of a novel bioinformatically detected viral gene of the present invention, referred to here as Viral Genomic Address Messenger 63 (VGAM63) viral gene, which modulates expression of respective host target genes thereof, the function and utility of which host target genes is known in the art.

VGAM63 is a novel bioinformatically detected regulatory, non protein coding, viral micro RNA (miRNA) gene. The method by which VGAM63 was detected is described hereinabove with reference to FIGS. 1-8.

VGAM63 gene, herein designated VGAM GENE, is a viral gene contained in the genome of Vaccinia Virus. VGAM63 host target gene, herein designated VGAM HOST TARGET GENE, is a human gene contained in the human genome.

VGAM63 gene encodes a VGAM63 precursor RNA, herein designated VGAM PRECURSOR RNA. Similar to other miRNA genes, and unlike most ordinary genes, VGAM63 precursor RNA does not encode a protein. A nucleotide sequence identical or highly similar to the nucleotide sequence of VGAM63 precursor RNA is designated SEQ ID:49, and is provided hereinbelow with reference to the sequence listing part. Nucleotide sequence SEQ ID:49 is located at position 14074 relative to the genome of Vaccinia Virus.

VGAM63 precursor RNA folds onto itself, forming VGAM63 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM63 folded precursor RNA into VGAM63 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM63 RNA is designated SEQ ID:398, and is provided hereinbelow with reference to the sequence listing part.

VGAM63 host target gene, herein designated VGAM HOST TARGET GENE, encodes a corresponding messenger RNA, VGAM63 host target RNA, herein designated VGAM HOST TARGET RNA. VGAM63 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM63 RNA, herein designated VGAM RNA, binds complementarily to one or more host target binding sites located in untranslated regions of VGAM63 host target RNA, herein designated VGAM HOST TARGET RNA. This complementary binding is due to the fact that the nucleotide sequence of VGAM63 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM63 RNA, herein designated VGAM RNA, may have a different number of host target binding sites in untranslated regions of a VGAM63 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM63 RNA, herein designated VGAM RNA, to host target binding sites on VGAM63 host target RNA, herein designated VGAM HOST TARGET RNA, such as BINDING SITE I, BINDING SITE II and BINDING SITE III, inhibits translation of VGAM63 host target RNA into VGAM63 host target protein, herein designated VGAM HOST TARGET PROTEIN. VGAM host target protein is therefore outlined by a broken line.

It is appreciated that VGAM63 host target gene, herein designated VGAM HOST TARGET GENE, in fact represents a plurality of VGAM63 host target genes. The mRNA of each one of this plurality of VGAM63 host target genes comprises one or more host target binding sites, each having a nucleotide sequence which is at least partly complementary to VGAM63 RNA, herein designated VGAM RNA, and which when bound by VGAM63 RNA causes inhibition of translation of respective one or more VGAM63 host target proteins.

It is further appreciated by one skilled in the art that the mode of translational inhibition illustrated by FIG. 1 with specific reference to translational inhibition exerted by VGAM63 gene, herein designated VGAM GENE, on one or more VGAM63 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM63 is inhibition of expression of host target genes, as part of a novel viral mechanism of attacking a host. Accordingly, utilities of VGAM63 include diagnosis, prevention and treatment of viral infection by Vaccinia Virus. Specific functions, and accordingly utilities, of VGAM63 correlate with, and may be deduced from, the identity of the host target genes which VGAM63 binds and inhibits, and the function of these host target genes, as elaborated hereinbelow.

Nucleotide sequences of the VGAM63 precursor RNA, herein designated VGAM PRECURSOR RNA, and of the ‘diced’ VGAM63 RNA, herein designated VGAM RNA, and a schematic representation of the secondary folding of VGAM63 folded precursor RNA, herein designated VGAM FOLDED PRECURSOR RNA, of VGAM63 are further described hereinbelow with reference to Table 1.

Nucleotide sequences of host target binding sites, such as BINDING SITE-I, BINDING SITE-II and BINDING SITE-III of FIG. 1, found on VGAM63 host target RNA, and schematic representation of the complementarity of each of these host target binding sites to VGAM63 RNA, herein designated VGAM RNA, are described hereinbelow with reference to Table 2.

As mentioned hereinabove with reference to FIG. 1, a function of VGAM63 gene, herein designated VGAM is inhibition of expression of VGAM63 target genes. It is appreciated that specific functions, and accordingly utilities, of VGAM63 correlate with, and may be deduced from, the identity of the target genes which VGAM63 binds and inhibits, and the function of these target genes, as elaborated hereinbelow.

FLJ10737 (Accession NM_{—}018198) is a VGAM63 host target gene. FLJ10737 BINDING SITE is HOST TARGET binding site found in the 3′ untranslated region of mRNA encoded by FLJ10737, corresponding to a HOST TARGET binding site such as BINDING SITE I, BINDING SITE II or BINDING SITE III. Table 2 illustrates the complementarity of the nucleotide sequences of FLJ10737 BINDING SITE, designated SEQ ID: 1797, to the nucleotide sequence of VGAM63 RNA, herein designated VGAM RNA, also designated SEQ ID:398.

A function of VGAM63 is therefore inhibition of FLJ10737 (Accession NM_{—}018198). Accordingly, utilities of VGAM63 include diagnosis, prevention and treatment of diseases and clinical conditions associated with FLJ10737. FIG. 1 further provides a conceptual description of a novel bioinformatically detected viral gene of the present invention, referred to here as Viral Genomic Address Messenger 64 (VGAM64) viral gene, which modulates expression of respective host target genes thereof, the function and utility of which host target genes is known in the art.

VGAM64 is a novel bioinformatically detected regulatory, non protein coding, viral micro RNA (miRNA) gene. The method by which VGAM64 was detected is described hereinabove with reference to FIGS. 1-8.

VGAM64 gene, herein designated VGAM GENE, is a viral gene contained in the genome of Vaccinia Virus. VGAM64 host target gene, herein designated VGAM HOST TARGET GENE, is a human gene contained in the human genome.

VGAM64 gene encodes a VGAM64 precursor RNA, herein designated VGAM PRECURSOR RNA. Similar to other miRNA genes, and unlike most ordinary genes, VGAM64 precursor RNA does not encode a protein. A nucleotide sequence identical or highly similar to the nucleotide sequence of VGAM64 precursor RNA is designated SEQ ID:50, and is provided hereinbelow with reference to the sequence listing part. Nucleotide sequence SEQ ID:50 is located at position 9005 relative to the genome of Vaccinia Virus.

VGAM64 precursor RNA folds onto itself, forming VGAM64 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM64 folded precursor RNA into VGAM64 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM64 RNA is designated SEQ ID:399, and is provided hereinbelow with reference to the sequence listing part.

VGAM64 host target gene, herein designated VGAM HOST TARGET GENE, encodes a corresponding messenger RNA, VGAM64 host target RNA, herein designated VGAM HOST TARGET RNA. VGAM64 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM64 RNA, herein designated VGAM RNA, binds complementarily to one or more host target binding sites located in untranslated regions of VGAM64 host target RNA, herein designated VGAM HOST TARGET RNA. This complementary binding is due to the fact that the nucleotide sequence of VGAM64 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM64 RNA, herein designated VGAM RNA, may have a different number of host target binding sites in untranslated regions of a VGAM64 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM64 RNA, herein designated VGAM RNA, to host target binding sites on VGAM64 host target RNA, herein designated VGAM HOST TARGET RNA, such as BINDING SITE I, BINDING SITE II and BINDING SITE III, inhibits translation of VGAM64 host target RNA into VGAM64 host target protein, herein designated VGAM HOST TARGET PROTEIN. VGAM host target protein is therefore outlined by a broken line.

It is appreciated that VGAM64 host target gene, herein designated VGAM HOST TARGET GENE, in fact represents a plurality of VGAM64 host target genes. The mRNA of each one of this plurality of VGAM64 host target genes comprises one or more host target binding sites, each having a nucleotide sequence which is at least partly complementary to VGAM64 RNA, herein designated VGAM RNA, and which when bound by VGAM64 RNA causes inhibition of translation of respective one or more VGAM64 host target proteins.

It is further appreciated by one skilled in the art that the mode of translational inhibition illustrated by FIG. 1 with specific reference to translational inhibition exerted by VGAM64 gene, herein designated VGAM GENE, on one or more VGAM64 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM64 is inhibition of expression of host target genes, as part of a novel viral mechanism of attacking a host. Accordingly, utilities of VGAM64 include diagnosis, prevention and treatment of viral infection by Vaccinia Virus. Specific functions, and accordingly utilities, of VGAM64 correlate with, and may be deduced from, the identity of the host target genes which VGAM64 binds and inhibits, and the function of these host target genes, as elaborated hereinbelow.

Nucleotide sequences of the VGAM64 precursor RNA, herein designated VGAM PRECURSOR RNA, and of the ‘diced’ VGAM64 RNA, herein designated VGAM RNA, and a schematic representation of the secondary folding of VGAM64 folded precursor RNA, herein designated VGAM FOLDED PRECURSOR RNA, of VGAM64 are further described hereinbelow with reference to Table 1.

Nucleotide sequences of host target binding sites, such as BINDING SITE-I, BINDING SITE-II and BINDING SITE-III of FIG. 1, found on VGAM64 host target RNA, and schematic representation of the complementarity of each of these host target binding sites to VGAM64 RNA, herein designated VGAM RNA, are described hereinbelow with reference to Table 2.

As mentioned hereinabove with reference to FIG. 1, a function of VGAM64 gene, herein designated VGAM is inhibition of expression of VGAM64 target genes. It is appreciated that specific functions, and accordingly utilities, of VGAM64 correlate with, and may be deduced from, the identity of the target genes which VGAM64 binds and inhibits, and the function of these target genes, as elaborated hereinbelow.

COX15 Homolog, Cytochrome C Oxidase Assembly Protein (yeast) (COX15, Accession NM_{—}004376) is a VGAM64 host target gene. COX15 BINDING SITE is HOST TARGET binding site found in the 3′ untranslated region of mRNA encoded by COX15, corresponding to a HOST TARGET binding site such as BINDING SITE I, BINDING SITE II or BINDING SITE III. Table 2 illustrates the complementarity of the nucleotide sequences of COX15 BINDING SITE, designated SEQ ID:1104, to the nucleotide sequence of VGAM64 RNA, herein designated VGAM RNA, also designated SEQ ID:399.

A function of VGAM64 is therefore inhibition of COX15 Homolog, Cytochrome C Oxidase Assembly Protein (yeast) (COX15, Accession NM_{—}004376). Accordingly, utilities of VGAM64 include diagnosis, prevention and treatment of diseases and clinical conditions associated with COX15. SH2 Domain Protein 1A, Duncan's Disease (lymphoproliferative syndrome) (SH2D1A, Accession NM_{—}002351) is another VGAM64 host target gene. SH2D1A BINDING SITE is HOST TARGET binding site found in the 3′ untranslated region of mRNA encoded by SH2D1A, corresponding to a HOST TARGET binding site such as BINDING SITE I, BINDING SITE II or BINDING SITE III. Table 2 illustrates the complementarity of the nucleotide sequences of SH2D1A BINDING SITE, designated SEQ ID:920, to the nucleotide sequence of VGAM64 RNA, herein designated VGAM RNA, also designated SEQ ID:399.

Another function of VGAM64 is therefore inhibition of SH2 Domain Protein 1A, Duncan's Disease (lymphoproliferative syndrome) (SH2D1A, Accession NM_{—}002351), a gene which is involved in t cell signaling. inhibits slam self-association. Accordingly, utilities of VGAM64 include diagnosis, prevention and treatment of diseases and clinical conditions associated with SH2D1A. The function of SH2D1A has been established by previous studies. Sumegi et al. (2000) reported that analysis of 35 families from the XLP Registry revealed 28 different mutations in 34 families: 3 large genomic deletions, 10 small intragenic deletions, 3 splice site, 3 nonsense, and 9 missense mutations. No mutations were found in 25 males, so-called sporadic XLP (males with an XLP phenotype after EBV infection but no family history of XLP), or in 9 patients with chronic active EBV syndrome. The authors found that although EBV infection often resulted in fulminant infectious mononucleosis, it was not necessary for the expression of other manifestations of XLP and correlated poorly with outcome. They interpreted the results as suggesting that unidentified factors, either environmental or genetic (e.g., modifier genes), contribute to the pathogenesis of XLP. Animal model experiments lend further support to the function of SH2D1A. Wu et al. (2001) generated Sap-deficient mice, which were fertile and had no defects in lymphocyte surface markers or overall morphology. Sap-deficient mice had increased lymphocytic choriomeningitis virus (LCMV)-specific splenic and hepatic T cells and increased gamma-interferon (IFNG; 147570) production compared with their wildtype littermates. All Sap-deficient mice died as a result of hepatotropic LCMV infection, while only 30% of wildtype mice died. In contrast to the increased Ifng production, interleukin-4 (IL4; 147780) production was markedly lower in Sap-deficient mice. Mice with a BALB/c background are normally highly susceptible to infection with the Leishmania major parasite due to poor Ifng production. However, Sap-deficient mice with a BALB/c background produced little 114 and high levels of Ifng and had lower parasite burdens than wildtype BALB/c mice. This suggested that in the absence of SAP, IL4 gene activation is defective. Lower 114 expression in Sap-deficient mice correlated with greatly reduced IgE production and reduced basal IgE expression. Wu et al. (2001) proposed that the Sap-deficient mouse model would be a useful tool for dissecting the complex XLP phenotypes.

It is appreciated that the abovementioned animal model for SH2D1A is acknowledged by those skilled in the art as a scientifically valid animal model, as can be further appreciated from the publications sited hereinbelow.

Full details of the abovementioned studies are described in the following publications, the disclosure of which are hereby incorporated by reference:
Sumegi, J.; Huang, D.; Lanyi, A.; Davis, J. D.; Seemayer, T. A.; Maeda, A.; Klein, G.; Seri, M.; Wakiguchi, H.; Purtilo, D. T.; Gross, T. G.: Correlation of mutations of the SH2D1A gene and Epstein-Barr virus infection with clinical phenotype and outcome in X-linked lymphoproliferative disease. Blood 96: 3118-3125, 2000.; and
Wu, C.; Nguyen, K. B.; Pien, G. C.; Wang, N.; Gullo, C.; Duncan, H.; Sosa, M. R.; Edwards, M. J.; Borrow, P.; Satoskar, A. R.; Sharpe, A. H.; Biron, C. A.; Terhorst, C.: SAP controls T c.

Further studies establishing the function and utilities of SH2D1A are found in John Hopkins OMIM database record ID 308240, and in sited publications numbered 1942-1945, 1947, 1948-1960, 942, 1827-1842, 2135-1850, 1978-30 and 1979-1989 listed in the bibliography section hereinbelow, which are also hereby incorporated by reference. FLJ12994 (Accession NM_{—}022841) is another VGAM64 host target gene. FLJ12994 BINDING SITE1 and FLJ12994 BINDING SITE2 are HOST TARGET binding sites found in untranslated regions of mRNA encoded by FLJ12994, corresponding to HOST TARGET binding sites such as BINDING SITE I, BINDING SITE II or BINDING SITE III. Table 2 illustrates the complementarity of the nucleotide sequences of FLJ12994 BINDING SITE1 and FLJ12994 BINDING SITE2, designated SEQ ID:2017 and SEQ ID:3081 respectively, to the nucleotide sequence of VGAM64 RNA, herein designated VGAM RNA, also designated SEQ ID:399.

Another function of VGAM64 is therefore inhibition of FLJ12994 (Accession NM_{—}022841). Accordingly, utilities of VGAM64 include diagnosis, prevention and treatment of diseases and clinical conditions associated with FLJ12994. Fibroblast Growth Factor Receptor 2 (bacteria-expressed kinase, keratinocyte growth factor receptor, craniofacial dysostosis 1, Crouzon syndrome, Pfeiffer syndrome, Jackson-Weiss syndrome) (FGFR2, Accession NM_{—}022969) is another VGAM65 host target gene. FGFR2 BINDING SITE1 through FGFR2 BINDING SITE6 are HOST TARGET binding sites found in untranslated regions of mRNA encoded by FGFR2, corresponding to HOST TARGET binding sites such as BINDING SITE I, BINDING SITE II or BINDING SITE III. Table 2 illustrates the complementarity of the nucleotide sequences of FGFR2 BINDING SITE1 through FGFR2 BINDING SITE6, designated SEQ ID:2023, SEQ ID:2024, SEQ ID:2026, SEQ ID:2027, SEQ ID:2028 and SEQ ID:2010 respectively, to the nucleotide sequence of VGAM65 RNA, herein designated VGAM RNA, also designated SEQ ID:400.

Another function of VGAM65 is therefore inhibition of Fibroblast Growth Factor Receptor 2 (bacteria-expressed kinase, keratinocyte growth factor receptor, craniofacial dysostosis 1, Crouzon syndrome, Pfeiffer syndrome, Jackson-Weiss syndrome) (FGFR2, Accession NM_{—}022969). Accordingly, utilities of VGAM65 include diagnosis, prevention and treatment of diseases and clinical conditions associated with FGFR2. OS4 (Accession NM_{—}005730) is another VGAM66 host target gene. OS4 BINDING SITE is HOST TARGET binding site found in the 3′ untranslated region of mRNA encoded by OS4, corresponding to a HOST TARGET binding site such as BINDING SITE I, BINDING SITE II or BINDING SITE III. Table 2 illustrates the complementarity of the nucleotide sequences of OS4 BINDING SITE, designated SEQ ID:1239, to the nucleotide sequence of VGAM66 RNA, herein designated VGAM RNA, also designated SEQ ID:401.

Another function of VGAM66 is therefore inhibition of OS4 (Accession NM_{—}005730). Accordingly, utilities of VGAM66 include diagnosis, prevention and treatment of diseases and clinical conditions associated with OS4. LOC148289 (Accession XM_{—}097437) is another VGAM66 host target gene. LOC148289 BINDING SITE is HOST TARGET binding site found in the 3′ untranslated region of mRNA encoded by LOC148289, corresponding to a HOST TARGET binding site such as BINDING SITE I, BINDING SITE II or BINDING SITE III. Table 2 illustrates the complementarity of the nucleotide sequences of LOC148289 BINDING SITE, designated SEQ ID:3300, to the nucleotide sequence of VGAM66 RNA, herein designated VGAM RNA, also designated SEQ ID:401.

Another function of VGAM66 is therefore inhibition of LOC148289 (Accession XM_{—}097437). Accordingly, utilities of VGAM66 include diagnosis, prevention and treatment of diseases and clinical conditions associated with LOC148289. LOC253160 (Accession XM_{—}173016) is another VGAM66 host target gene. LOC253160 BINDING SITE is HOST TARGET binding site found in the 3′ untranslated region of mRNA encoded by LOC253160, corresponding to a HOST TARGET binding site such as BINDING SITE I, BINDING SITE II or BINDING SITE III. Table 2 illustrates the complementarity of the nucleotide sequences of LOC253160 BINDING SITE, designated SEQ ID:3714, to the nucleotide sequence of VGAM66 RNA, herein designated VGAM RNA, also designated SEQ ID:401.

Another function of VGAM66 is therefore inhibition of LOC253160 (Accession XM_{—}173016). Accordingly, utilities of VGAM66 include diagnosis, prevention and treatment of diseases and clinical conditions associated with LOC253160. FIG. 1 further provides a conceptual description of a novel bioinformatically detected viral gene of the present invention, referred to here as Viral Genomic Address Messenger 67 (VGAM67) viral gene, which modulates expression of respective host target genes thereof, the function and utility of which host target genes is known in the art.

VGAM67 is a novel bioinformatically detected regulatory, non protein coding, viral micro RNA (miRNA) gene. The method by which VGAM67 was detected is described hereinabove with reference to FIGS. 1-8.

VGAM67 gene, herein designated VGAM GENE, is a viral gene contained in the genome of Vaccinia Virus. VGAM67 host target gene, herein designated VGAM HOST TARGET GENE, is a human gene contained in the human genome.

VGAM67 gene encodes a VGAM67 precursor RNA, herein designated VGAM PRECURSOR RNA. Similar to other miRNA genes, and unlike most ordinary genes, VGAM67 precursor RNA does not encode a protein. A nucleotide sequence identical or highly similar to the nucleotide sequence of VGAM67 precursor RNA is designated SEQ ID:53, and is provided hereinbelow with reference to the sequence listing part. Nucleotide sequence SEQ ID:53 is located at position 12981 relative to the genome of Vaccinia Virus.

VGAM67 precursor RNA folds onto itself, forming VGAM67 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM67 folded precursor RNA into VGAM67 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM67 RNA is designated SEQ ID:402, and is provided hereinbelow with reference to the sequence listing part.

VGAM67 host target gene, herein designated VGAM HOST TARGET GENE, encodes a corresponding messenger RNA, VGAM67 host target RNA, herein designated VGAM HOST TARGET RNA. VGAM67 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM67 RNA, herein designated VGAM RNA, binds complementarily to one or more host target binding sites located in untranslated regions of VGAM67 host target RNA, herein designated VGAM HOST TARGET RNA. This complementary binding is due to the fact that the nucleotide sequence of VGAM67 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM67 RNA, herein designated VGAM RNA, may have a different number of host target binding sites in untranslated regions of a VGAM67 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM67 RNA, herein designated VGAM RNA, to host target binding sites on VGAM67 host target RNA, herein designated VGAM HOST TARGET RNA, such as BINDING SITE I, BINDING SITE II and BINDING SITE III, inhibits translation of VGAM67 host target RNA into VGAM67 host target protein, herein designated VGAM HOST TARGET PROTEIN. VGAM host target protein is therefore outlined by a broken line.

It is appreciated that VGAM67 host target gene, herein designated VGAM HOST TARGET GENE, in fact represents a plurality of VGAM67 host target genes. The mRNA of each one of this plurality of VGAM67 host target genes comprises one or more host target binding sites, each having a nucleotide sequence which is at least partly complementary to VGAM67 RNA, herein designated VGAM RNA, and which when bound by VGAM67 RNA causes inhibition of translation of respective one or more VGAM67 host target proteins.

It is further appreciated by one skilled in the art that the mode of translational inhibition illustrated by FIG. 1 with specific reference to translational inhibition exerted by VGAM67 gene, herein designated VGAM GENE, on one or more VGAM67 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM67 is inhibition of expression of host target genes, as part of a novel viral mechanism of attacking a host. Accordingly, utilities of VGAM67 include diagnosis, prevention and treatment of viral infection by Vaccinia Virus. Specific functions, and accordingly utilities, of VGAM67 correlate with, and may be deduced from, the identity of the host target genes which VGAM67 binds and inhibits, and the function of these host target genes, as elaborated hereinbelow.

Nucleotide sequences of the VGAM67 precursor RNA, herein designated VGAM PRECURSOR RNA, and of the ‘diced’ VGAM67 RNA, herein designated VGAM RNA, and a schematic representation of the secondary folding of VGAM67 folded precursor RNA, herein designated VGAM FOLDED PRECURSOR RNA, of VGAM67 are further described hereinbelow with reference to Table 1.

Nucleotide sequences of host target binding sites, such as BINDING SITE-I, BINDING SITE-II and BINDING SITE-III of FIG. 1, found on VGAM67 host target RNA, and schematic representation of the complementarity of each of these host target binding sites to VGAM67 RNA, herein designated VGAM RNA, are described hereinbelow with reference to Table 2.

As mentioned hereinabove with reference to FIG. 1, a function of VGAM67 gene, herein designated VGAM is inhibition of expression of VGAM67 target genes. It is appreciated that specific functions, and accordingly utilities, of VGAM67 correlate with, and may be deduced from, the identity of the target genes which VGAM67 binds and inhibits, and the function of these target genes, as elaborated hereinbelow.

Giant Axonal Neuropathy (gigaxonin) (GAN, Accession NM_{—}022041) is a VGAM67 host target gene. GAN BINDING SITE is HOST TARGET binding site found in the 3′ untranslated region of mRNA encoded by GAN, corresponding to a HOST TARGET binding site such as BINDING SITE I, BINDING SITE II or BINDING SITE III. Table 2 illustrates the complementarity of the nucleotide sequences of GAN BINDING SITE, designated SEQ ID:1975, to the nucleotide sequence of VGAM67 RNA, herein designated VGAM RNA, also designated SEQ ID:402.

A function of VGAM67 is therefore inhibition of Giant Axonal Neuropathy (gigaxonin) (GAN, Accession NM_{—}022041), a gene which plays an important role in neurofilament architecture. Accordingly, utilities of VGAM67 include diagnosis, prevention and treatment of diseases and clinical conditions associated with GAN. The function of GAN has been established by previous studies. Giant axonal neuropathy (GAN; 256850), a severe autosomal recessive sensorineural neuropathy affecting both the peripheral nerves and the central nervous system, is characterized by neurofilament accumulation, leading to segmental distention of axons. The neuropathy is part of a generalized disorganization of the cytoskeletal intermediate filaments (IFs), to which neurofilaments belong, as abnormal aggregation of multiple tissue-specific IFs has been reported in this disorder: vimentin (VIM; 193060) in endothelial cells, Schwann cells, and cultured skin fibroblasts, and glial fibrillary acidic protein (GFAP; 137780) in astrocytes (Prineas et al., 1976; Pena, 1982; Bousquet et al., 1996). Keratin intermediate filaments also seem to be altered, as most patients present characteristic curly or kinky hairs (Treiber-Held et al., 1994). Bomont et al. (2000) used a positional cloning approach to isolate a novel, ubiquitously expressed gene that encoded a protein they named gigaxonin and contained mutations associated with giant axonal neuropathy. Gigaxonin contains an N-terminal BTB (broad-complex, tramtrack, and brica-brac) domain followed by 6 kelch repeats, which were predicted to adopt a beta-propeller shape. Distantly related proteins sharing a similar domain organization have various functions associated with the cytoskeleton, predicting that gigaxonin is a novel and distinct cytoskeletal protein that may represent a general pathologic target for other neurodegenerative disorders with alterations in the neurofilament network.

Full details of the abovementioned studies are described in the following publications, the disclosure of which are hereby incorporated by reference:
Bomont, P.; Cavalier, L.; Blondeau, F.; Ben Hamida, C.; Belal, S.; Tazir, M.; Demir, E.; Topaloglu, H.; Korinthenberg, R.; Tuysuz, B.; Landrieu, P.; Hentati, F.; Koenig, M.: The gene encoding gigaxonin, a new member of the cytoskeletal BTB/kelch repeat family, is mutated in giant axonal neuropathy. Nature Genet. 26: 370-374, 2000.; and
Bousquet, O.; Basseville, M.; Vila-Porcile, E.; Billette de Villemeur, T.; Hauw, J.-J.; Landrieu, P.; Portier, M.-M.: Aggregation of a subpopulation of vimentin filaments in cultured.

Further studies establishing the function and utilities of GAN are found in John Hopkins OMIM database record ID 605379, and in sited publications numbered 2067-161 and 2068 listed in the bibliography section hereinbelow, which are also hereby incorporated by reference. Isocitrate Dehydrogenase 3 (NAD+) Alpha (IDH3A, Accession NM_{—}005530) is another VGAM67 host target gene. IDH3A BINDING SITE is HOST TARGET binding site found in the 3′ untranslated region of mRNA encoded by IDH3A, corresponding to a HOST TARGET binding site such as BINDING SITE I, BINDING SITE II or BINDING SITE III. Table 2 illustrates the complementarity of the nucleotide sequences of IDH3A BINDING SITE, designated SEQ ID:1222, to the nucleotide sequence of VGAM67 RNA, herein designated VGAM RNA, also designated SEQ ID:402.

Another function of VGAM67 is therefore inhibition of Isocitrate Dehydrogenase 3 (NAD+) Alpha (IDH3A, Accession NM_{—}005530), a gene which decarboxylates isocitrate into alpha-ketoglutarate in the TCA cycle. Accordingly, utilities of VGAM67 include diagnosis, prevention and treatment of diseases and clinical conditions associated with IDH3A. The function of IDH3A has been established by previous studies. Isocitrate dehydrogenases (EC 1.1.1.4.1 and EC 1.1.1.42) catalyze the oxidative decarboxylation of isocitrate into alpha-ketoglutarate. Thus, they are key enzymes in the tricarboxylic acid (TCA) cycle. In mammalian tissues, 3 kinds of isoenzymes exist, cytosolic NADP(+)-specific IDH (OMIM Ref. No. 147700), mitochondrial NADP(+)-specific IDH (OMIM Ref. No. 147650), and mitochondrial NAD(+)-specific IDH (IDH3). Among the mammalian IDH isoenzymes, IDH3 is thought to play a major role in isocitrate decarboxylation in the TCA cycle, since its activity is regulated by numerous allosteric regulators. IDH3, purified to homogeneity from pig heart, is a heterotetramer of the 2-alpha, 1-beta, and 1-gamma subunits, while NADP(+)-specific IDH found in either mitochondria or cytosol is a homodimer. Kim et al. (1995) characterized the cDNA clone for the alpha subunit (IDH3A), which encodes a mature protein with 339 amino acids (36,640 Da). Kim et al. (1995) had found that the human IDH3A sequence showed 44 and 30% amino acid identity to the monkey IDH3G (OMIM Ref. No. 602017) and bovine IDH2 (OMIM Ref. No. 147650) genes, respectively. Preliminary data using fluorescence in situ hybridization (FISH) supported the assignment to 15q26.1. By FISH, Huh et al. (1996) mapped the IDH3A gene to 15q25.1-q25.2

Full details of the abovementioned studies are described in the following publications, the disclosure of which are hereby incorporated by reference:
Huh, T.-L.; Kim, Y.-O.; Oh, I.-U.; Song, B. J.; Inazawa, J.: Assignment of the human mitochondrial NAD(+)-specific isocitrate dehydrogenase alpha subunit (IDH3A) gene to 15q25.1-q25.2 by in situ hybridization. Genomics 31: 295-296, 1996.; and
Kim, Y.-O.; Oh, I.-U.; Park, H.-S.; Jeng, J.; Song, B. J.; Huh, T.-L.: Characterization of a cDNA clone for human NAD(+)-specific isocitrate dehydrogenase alpha-subunit and structural c.

Further studies establishing the function and utilities of IDH3A are found in John Hopkins OMIM database record ID 601149, and in sited publications numbered 1705-1706 listed in the bibliography section hereinbelow, which are also hereby incorporated by reference. Peroxisome Biogenesis Factor 1 (PEX1, Accession NM_{—}000466) is another VGAM67 host target gene. PEX1 BINDING SITE is HOST TARGET binding site found in the 3′ untranslated region of mRNA encoded by PEX1, corresponding to a HOST TARGET binding site such as BINDING SITE I, BINDING SITE II or BINDING SITE III. Table 2 illustrates the complementarity of the nucleotide sequences of PEX1 BINDING SITE, designated SEQ ID:749, to the nucleotide sequence of VGAM67 RNA, herein designated VGAM RNA, also designated SEQ ID:402.

Another function of VGAM67 is therefore inhibition of Peroxisome Biogenesis Factor 1 (PEX1, Accession NM_{—}000466). Accordingly, utilities of VGAM67 include diagnosis, prevention and treatment of diseases and clinical conditions associated with PEX1. PYGO2 (Accession XM_{—}034083) is another VGAM67 host target gene. PYGO2 BINDING SITE is HOST TARGET binding site found in the 3′ untranslated region of mRNA encoded by PYGO2, corresponding to a HOST TARGET binding site such as BINDING SITE I, BINDING SITE II or BINDING SITE III. Table 2 illustrates the complementarity of the nucleotide sequences of PYGO2 BINDING SITE, designated SEQ ID:2676, to the nucleotide sequence of VGAM67 RNA, herein designated VGAM RNA, also designated SEQ ID:402.

Another function of VGAM67 is therefore inhibition of PYGO2 (Accession XM_{—}034083). Accordingly, utilities of VGAM67 include diagnosis, prevention and treatment of diseases and clinical conditions associated with PYGO2. FLJ21477 (Accession NM_{—}025153) is another VGAM67 host target gene. FLJ21477 BINDING SITE is HOST TARGET binding site found in the 3′ untranslated region of mRNA encoded by FLJ21477, corresponding to a HOST TARGET binding site such as BINDING SITE I, BINDING SITE II or BINDING SITE III. Table 2 illustrates the complementarity of the nucleotide sequences of FLJ21477 BINDING SITE, designated SEQ ID:2142, to the nucleotide sequence of VGAM67 RNA, herein designated VGAM RNA, also designated SEQ ID:402.

Another function of VGAM67 is therefore inhibition of FLJ21477 (Accession NM_{—}025153). Accordingly, utilities of VGAM67 include diagnosis, prevention and treatment of diseases and clinical conditions associated with FLJ21477. NS1-BP (Accession XM_{—}051877) is another VGAM67 host target gene. NS1-BP BINDING SITE is HOST TARGET binding site found in the 3′ untranslated region of mRNA encoded by NS1-BP, corresponding to a HOST TARGET binding site such as BINDING SITE I, BINDING SITE II or BINDING SITE III. Table 2 illustrates the complementarity of the nucleotide sequences of NS1-BP BINDING SITE, designated SEQ ID:2953, to the nucleotide sequence of VGAM67 RNA, herein designated VGAM RNA, also designated SEQ ID:402.

Another function of VGAM67 is therefore inhibition of NS1-BP (Accession XM_{—}051877). Accordingly, utilities of VGAM67 include diagnosis, prevention and treatment of diseases and clinical conditions associated with NS1-BP. Unc-5 Homolog D (C. elegans) (UNC5D, Accession NM_{—}080872) is another VGAM67 host target gene. UNC5D BINDING SITE is HOST TARGET binding site found in the 5′ untranslated region of mRNA encoded by UNC5D, corresponding to a HOST TARGET binding site such as BINDING SITE I, BINDING SITE II or BINDING SITE III. Table 2 illustrates the complementarity of the nucleotide sequences of UNC5D BINDING SITE, designated SEQ ID:2391, to the nucleotide sequence of VGAM67 RNA, herein designated VGAM RNA, also designated SEQ ID:402.

Another function of VGAM67 is therefore inhibition of Unc-5 Homolog D (C. elegans) (UNC5D, Accession NM_{—}080872). Accordingly, utilities of VGAM67 include diagnosis, prevention and treatment of diseases and clinical conditions associated with UNC5D. LOC257451 (Accession XM_{—}170960) is another VGAM67 host target gene. LOC257451 BINDING SITE is HOST TARGET binding site found in the 3′ untranslated region of mRNA encoded by LOC257451, corresponding to a HOST TARGET binding site such as BINDING SITE I, BINDING SITE II or BINDING SITE III. Table 2 illustrates the complementarity of the nucleotide sequences of LOC257451 BINDING SITE, designated SEQ ID:3678, to the nucleotide sequence of VGAM67 RNA, herein designated VGAM RNA, also designated SEQ ID:402.

Another function of VGAM67 is therefore inhibition of LOC257451 (Accession XM_{—}170960). Accordingly, utilities of VGAM67 include diagnosis, prevention and treatment of diseases and clinical conditions associated with LOC257451. FIG. 1 further provides a conceptual description of a novel bioinformatically detected viral gene of the present invention, referred to here as Viral Genomic Address Messenger 68 (VGAM68) viral gene, which modulates expression of respective host target genes thereof, the function and utility of which host target genes is known in the art.

VGAM68 is a novel bioinformatically detected regulatory, non protein coding, viral micro RNA (miRNA) gene. The method by which VGAM68 was detected is described hereinabove with reference to FIGS. 1-8.

VGAM68 gene, herein designated VGAM GENE, is a viral gene contained in the genome of Vaccinia Virus. VGAM68 host target gene, herein designated VGAM HOST TARGET GENE, is a human gene contained in the human genome.

VGAM68 gene encodes a VGAM68 precursor RNA, herein designated VGAM PRECURSOR RNA. Similar to other miRNA genes, and unlike most ordinary genes, VGAM68 precursor RNA does not encode a protein. A nucleotide sequence identical or highly similar to the nucleotide sequence of VGAM68 precursor RNA is designated SEQ ID:54, and is provided hereinbelow with reference to the sequence listing part. Nucleotide sequence SEQ ID:54 is located at position 10620 relative to the genome of Vaccinia Virus.

VGAM68 precursor RNA folds onto itself, forming VGAM68 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM68 folded precursor RNA into VGAM68 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM68 RNA is designated SEQ ID:403, and is provided hereinbelow with reference to the sequence listing part.

VGAM68 host target gene, herein designated VGAM HOST TARGET GENE, encodes a corresponding messenger RNA, VGAM68 host target RNA, herein designated VGAM HOST TARGET RNA. VGAM68 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM68 RNA, herein designated VGAM RNA, binds complementarily to one or more host target binding sites located in untranslated regions of VGAM68 host target RNA, herein designated VGAM HOST TARGET RNA. This complementary binding is due to the fact that the nucleotide sequence of VGAM68 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM68 RNA, herein designated VGAM RNA, may have a different number of host target binding sites in untranslated regions of a VGAM68 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM68 RNA, herein designated VGAM RNA, to host target binding sites on VGAM68 host target RNA, herein designated VGAM HOST TARGET RNA, such as BINDING SITE I, BINDING SITE II and BINDING SITE III, inhibits translation of VGAM68 host target RNA into VGAM68 host target protein, herein designated VGAM HOST TARGET PROTEIN. VGAM host target protein is therefore outlined by a broken line.

It is appreciated that VGAM68 host target gene, herein designated VGAM HOST TARGET GENE, in fact represents a plurality of VGAM68 host target genes. The mRNA of each one of this plurality of VGAM68 host target genes comprises one or more host target binding sites, each having a nucleotide sequence which is at least partly complementary to VGAM68 RNA, herein designated VGAM RNA, and which when bound by VGAM68 RNA causes inhibition of translation of respective one or more VGAM68 host target proteins.

It is further appreciated by one skilled in the art that the mode of translational inhibition illustrated by FIG. 1 with specific reference to translational inhibition exerted by VGAM68 gene, herein designated VGAM GENE, on one or more VGAM68 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM68 is inhibition of expression of host target genes, as part of a novel viral mechanism of attacking a host. Accordingly, utilities of VGAM68 include diagnosis, prevention and treatment of viral infection by Vaccinia Virus. Specific functions, and accordingly utilities, of VGAM68 correlate with, and may be deduced from, the identity of the host target genes which VGAM68 binds and inhibits, and the function of these host target genes, as elaborated hereinbelow.

Nucleotide sequences of the VGAM68 precursor RNA, herein designated VGAM PRECURSOR RNA, and of the ‘diced’ VGAM68 RNA, herein designated VGAM RNA, and a schematic representation of the secondary folding of VGAM68 folded precursor RNA, herein designated VGAM FOLDED PRECURSOR RNA, of VGAM68 are further described hereinbelow with reference to Table 1.

Nucleotide sequences of host target binding sites, such as BINDING SITE-I, BINDING SITE-II and BINDING SITE-III of FIG. 1, found on VGAM68 host target RNA, and schematic representation of the complementarity of each of these host target binding sites to VGAM68 RNA, herein designated VGAM RNA, are described hereinbelow with reference to Table 2.

As mentioned hereinabove with reference to FIG. 1, a function of VGAM68 gene, herein designated VGAM is inhibition of expression of VGAM68 target genes. It is appreciated that specific functions, and accordingly utilities, of VGAM68 correlate with, and may be deduced from, the identity of the target genes which VGAM68 binds and inhibits, and the function of these target genes, as elaborated hereinbelow.

Chorea Acanthocytosis (CHAC, Accession NM_{—}015186) is a VGAM68 host target gene. CHAC BINDING SITE is HOST TARGET binding site found in the 3′ untranslated region of mRNA encoded by CHAC, corresponding to a HOST TARGET binding site such as BINDING SITE I, BINDING SITE II or BINDING SITE III. Table 2 illustrates the complementarity of the nucleotide sequences of CHAC BINDING SITE, designated SEQ ID: 1612, to the nucleotide sequence of VGAM68 RNA, herein designated VGAM RNA, also designated SEQ ID:403.

A function of VGAM68 is therefore inhibition of Chorea Acanthocytosis (CHAC, Accession NM_{—}015186), a gene which may regulate the cycling of proteins. Accordingly, utilities of VGAM68 include diagnosis, prevention and treatment of diseases and clinical conditions associated with CHAC. The function of CHAC has been established by previous studies. Rampoldi et al. (2001) identified a novel gene in the choreoacanthocytosis (CHAC; 200150) critical region of 9q with an open reading frame of 9,525 nucleotides encoding a 3,174-amino acid protein. Alignment of the RNA with the genomic sequence demonstrated that the gene is organized into 73 exons in a genomic region of about 250 kb. Northern blot analysis detected 2 bands of high molecular weight corresponding to 2 splice variants in all tissues analyzed. Additionally, RT-PCR detected expression in the erythrocyte precursor cell line K562. Rampoldi et al. (2001) found 16 different mutations in individuals with choreoacanthocytosis. They showed that the CHAC gene encodes an evolutionarily conserved protein and suggested that this protein is involved in protein sorting. In a patient with choreoacanthocytosis (OMIM Ref. No. 200150), Rampoldi et al. (2001) found compound heterozygosity for a 269T-A transversion in exon 4 of the CHAC gene and an insertion of a T between nucleotides 6404 and 6405 in exon 48. The mutations resulted in an ile90-to-lys (190K) amino acid change and a frameshift, respectively.

Full details of the abovementioned studies are described in the following publications, the disclosure of which are hereby incorporated by reference:
Rampoldi, L.; Dobson-Stone, C.; Rubio, J. P.; Danek, A.; Chalmers, R. M.; Wood, N. W.; Verellen, C.; Ferrer, X.; Malandrini, A.; Fabrizi, G. M.; Brown, R.; Vance, J.; Pericak-Vance, M.; Rudolf, G.; Carre, S.; Alonso, E.; Manfredi, M.; Nemeth, A. H.; Monaco, A. P.: A conserved sorting-associated protein is mutant in chorea-acanthocytosis. Nature Genet. 28: 119-120, 2001.; and
Ueno, S.; Maruki, Y.; Nakamura, M.; Tomemori, Y.; Kamae, K.; Tanabe, H.; Yamashita, Y.; Matsuda, S.; Kaneko, S.; Sano, A.: The gene encoding a newly discovered protein, chorein, is mu.

Further studies establishing the function and utilities of CHAC are found in John Hopkins OMIM database record ID 605978, and in sited publications numbered 2375 listed in the bibliography section hereinbelow, which are also hereby incorporated by reference. Gamma-glutamyl Carboxylase (GGCX, Accession NM_{—}000821) is another VGAM68 host target gene. GGCX BINDING SITE is HOST TARGET binding site found in the 3′ untranslated region of mRNA encoded by GGCX, corresponding to a HOST TARGET binding site such as BINDING SITE I, BINDING SITE II or BINDING SITE III. Table 2 illustrates the complementarity of the nucleotide sequences of GGCX BINDING SITE, designated SEQ ID:778, to the nucleotide sequence of VGAM68 RNA, herein designated VGAM RNA, also designated SEQ ID:403.

Another function of VGAM68 is therefore inhibition of Gamma-glutamyl Carboxylase (GGCX, Accession NM_{—}000821). Accordingly, utilities of VGAM68 include diagnosis, prevention and treatment of diseases and clinical conditions associated with GGCX. Inositol 1,4,5-trisphosphate 3-kinase B (ITPKB, Accession NM_{—}002221) is another VGAM68 host target gene. ITPKB BINDING SITE is HOST TARGET binding site found in the 3′ untranslated region of mRNA encoded by ITPKB, corresponding to a HOST TARGET binding site such as BINDING SITE I, BINDING SITE II or BINDING SITE III. Table 2 illustrates the complementarity of the nucleotide sequences of ITPKB BINDING SITE, designated SEQ ID:911, to the nucleotide sequence of VGAM68 RNA, herein designated VGAM RNA, also designated SEQ ID:403.

Another function of VGAM68 is therefore inhibition of Inositol 1,4,5-trisphosphate 3-kinase B (ITPKB, Accession NM_{—}002221), a gene which is a type B inositol 1,4,5-triphosphate 3 kinase. Accordingly, utilities of VGAM68 include diagnosis, prevention and treatment of diseases and clinical conditions associated with ITPKB. The function of ITPKB has been established by previous studies. Takazawa et al. (1991) isolated a second inositol 1,4,5-trisphosphate 3-kinase cDNA from a human hippocampus cDNA library. Sequencing yielded an open reading frame encoding a 472-amino acid protein with a calculated relative mass of 53,451. The C-terminal part of this enzyme, referred to as 3-kinase-B, namely, residues 187-462, was 68% identical to 3-kinase-A (OMIM Ref. No. 147521) in amino acid sequence. By in situ hybridization, Erneux et al. (1992) mapped the ITPKB gene to 1q41-q43.

Full details of the abovementioned studies are described in the following publications, the disclosure of which are hereby incorporated by reference:
Erneux, C.; Roeckel, N.; Takazawa, K.; Mailleux, P.; Vassart, G.; Mattei, M. G.: Localization of the genes for human inositol 1,4,5-trisphosphate 3-kinase A (ITPKA) and B (ITPKB) to chromosome regions 15q14-q21 and 1q41-q43, respectively, by in situ hybridization. Genomics 14: 546-547, 1992.; and
Takazawa, K.; Perret, J.; Dumont, J. E.; Erneux, C.: Molecular cloning and expression of a new putative inositol 1,4,5-trisphosphate 3-kinase isoenzyme. Biochem. J. 278: 883-886, 1991.

Further studies establishing the function and utilities of ITPKB are found in John Hopkins OMIM database record ID 147522, and in sited publications numbered 15-16 listed in the bibliography section hereinbelow, which are also hereby incorporated by reference. Potassium Voltage-gated Channel, Shaker-related Sub family, Beta Member 1 (KCNAB1, Accession XM_{—}027634) is another VGAM68 host target gene. KCNAB1 BINDING SITE is HOST TARGET binding site found in the 3′ untranslated region of mRNA encoded by KCNAB1, corresponding to a HOST TARGET binding site such as BINDING SITE I, BINDING SITE II or BINDING SITE III. Table 2 illustrates the complementarity of the nucleotide sequences of KCNAB1 BINDING SITE, designated SEQ ID:2584, to the nucleotide sequence of VGAM68 RNA, herein designated VGAM RNA, also designated SEQ ID:403.

Another function of VGAM68 is therefore inhibition of Potassium Voltage-gated Channel, Shaker-related Sub family, Beta Member 1 (KCNAB1, Accession XM_{—}027634), a gene which is the regulatory beta subunit for a shaker-related voltage-gated potassium channel. Accordingly, utilities of VGAM68 include diagnosis, prevention and treatment of diseases and clinical conditions associated with KCNAB1. The function of KCNAB1 has been established by previous studies. ‘Shaker’ and other voltage-dependent potassium channel proteins help to determine the electrical properties of excitable cells and play additional physiologic roles in nonexcitable cell types. Voltage-activated, outwardly rectifying potassium channels (Kv) are heterooligomers that are assembled from alpha and beta subunits in a 1:1 molar ratio. Schultz et al. (1996) noted that mammals contain a large number of alpha-subunit potassium channel genes, often clustered within the genome, that may have arisen through local and chromosomal duplication events. The associated beta subunits modulate the gating properties and amplitudes of the Shaker potassium currents. England et al. (1995) cloned a human heart cDNA encoding a beta subunit that they designated Kv-beta-1.3. Sequence analysis revealed that Kv-beta-1.3 and the previously identified human Kv-beta-1 (England et al., 1995) and Kv-beta-3 (McCormack et al., 1995) subunits differ only at their N termini and are encoded by alternatively spliced mRNAs from a single gene. The authors suggested that Kv-beta-1 and Kv-beta-3 be renamed Kv-beta-1.1 and Kv-beta-1.2, respectively. The predicted 419-amino acid Kv-beta-1.3 subunit does not contain a hydrophobic domain and is likely to be a cytoplasmic protein, like other beta subunits. When coexpressed in Xenopus oocytes, the Kv-beta-1.3 subunit altered the functional properties of Kv1.5 (KCNA5; 176267), converting it from a delayed rectifier to a channel with rapid but partial inactivation. In addition, Kv-beta-1.3 converted the Kv1.5 outwardly rectifying current-voltage relationship to one showing strong inward rectification. England et al. (1995) concluded that Kv channel current diversity may arise from association with alternatively spliced Kv-beta subunits. By Northern blot analysis, Leicher et al. (1996) found that the KCNA1B gene was expressed as 3.4- and 3.8-kb mRNAs in human brain. The pattern of expression of Kv1-alpha and Kv-beta subunits suggested an intricate and cell-specific regulatory mechanism that produces distinct combinations of alpha and beta subunits in different nuclei of the brain. The Kv-beta-1.1 and Kv-beta-1.2 splice variants contain an N-terminal inactivating domain similar to that found in A-type Kv channels (see OMIM Ref. No. KCNA4; 176266). When coexpressed in mammalian cells, Kv-beta-1.1 and Kv-beta-1.2 conferred rapid inactivation on Kv1.5 channels, with different potencies

Full details of the abovementioned studies are described in the following publications, the disclosure of which are hereby incorporated by reference:
Leicher, T. Roeper, J.; Weber, K.; Wang, X.; Pongs, O.: Structural and functional characterization of human potassium channel subunit beta-1 (KCNA1B). Neuropharmacology 35: 787-795, 1996.; and
Schultz, D.; Litt, M.; Smith, L.; Thayer, M.; McCormack, K.: Localization of two potassium channel beta subunit genes, KCNA1B and KCNA2B. Genomics 31: 389-391, 1996.

Further studies establishing the function and utilities of KCNAB1 are found in John Hopkins OMIM database record ID 601141, and in sited publications numbered 1592-656 listed in the bibliography section hereinbelow, which are also hereby incorporated by reference. Killer Cell Lectin-like Receptor Sub family C, Member 4 (KLRC4, Accession NM_{—}013431) is another VGAM68 host target gene. KLRC4 BINDING SITE is HOST TARGET binding site found in the 5′ untranslated region of mRNA encoded by KLRC4, corresponding to a HOST TARGET binding site such as BINDING SITE I, BINDING SITE II or BINDING SITE III. Table 2 illustrates the complementarity of the nucleotide sequences of KLRC4 BINDING SITE, designated SEQ ID:1451, to the nucleotide sequence of VGAM68 RNA, herein designated VGAM RNA, also designated SEQ ID:403.

Another function of VGAM68 is therefore inhibition of Killer Cell Lectin-like Receptor Sub family C, Member 4 (KLRC4, Accession NM_{—}013431), a gene which is a receptor for the recognition of mhc class i hla-e molecules by nk cells and some cytotoxic t-cells. Accordingly, utilities of VGAM68 include diagnosis, prevention and treatment of diseases and clinical conditions associated with KLRC4. The function of KLRC4 has been established by previous studies. Bauer et al. (1999) found that NKG2D is expressed on gamma/delta T cells, CD8-alpha (OMIM Ref. No. 186910)/-beta (OMIM Ref. No. 186730)-positive T cells, and natural killer cells and is a receptor for MICA (OMIM Ref. No. 600169). MICA binding to NKG2D activated cytolytic responses of gamma/delta T cells and NK cells against transfectants and epithelial tumor cells expressing MICA. The authors noted that these results define an activating immuno receptor-MHC ligand interaction that may promote antitumor NK and T-cell responses. Wu et al. (1999) demonstrated that NKG2D and DAP10 (OMIM Ref. No. 604089) interact specifically to form an activating immuno receptor complex. MIC engagement of NKG2D stimulates NK-cell and T-cell effector functions. Cytomegalovirus (CMV) infection induces the expression of stress proteins such as HSP70 (OMIM Ref. No. 140550). By flow cytometric analysis, Groh et al. (2001) showed that CMV infection also induces MIC expression and a concurrent downregulation of MHC class I molecules on fibroblasts and endothelial cells. Immunohistochemical analysis of lung sections from patients with CMV interstitial pneumonitis confirmed that induction of MIC expression also occurs in vivo. Functional analysis of T-cell cytotoxicity against CMV-infected fibroblasts showed that early after infection when MIC expression was low, antibodies to MHC class I, but not to MIC or NKG2D, could block T cell-mediated cytolysis. As MIC expression increased, antibody masking of MIC or NKG2D reduced target cell lysis; anti-MHC class I antibodies further reduced cytolysis. The presence of MICA on stimulator cells also substantially enhanced cytokine release by T-cell clones, and anti-MIC antibody abrogated this production, suggesting that the MIC-NKG2D interaction provides an important costimulatory activity. Animal model experiments lend further support to the function of KLRC4. Colucci et al. (2002) noted that humans with mutations in ZAP70 (OMIM Ref. No. 176947) have T-cell immunodeficiency, that mice lacking Zap70 have blocked T-cell development, and that mice lacking Syk (OMIM Ref. No. 600085) have a failure of B-cell development. NK cells express both molecules, which associate with immuno receptor tyrosine-based activation motifs (ITAMs). Using mice deficient in both Zap70 and Syk, Colucci et al. (2002) observed NK cell activity comparable to that in wildtype mice. The mutant cells expressed Nkg2d and were able to lyse targets with and without Nkg2d ligands in vitro and in vivo. However, wildtype cells, but not the double-deficient cells, responded to CD16 (OMIM Ref. No. 146740) and Ly49d (see OMIM Ref. No. 604274) cross-linking with increased cytotoxicity, suggesting that these 2 ITAM-bearing receptors are unable to signal in the mutant cells. Inhibitors of PI3K or Src kinases blocked and, in combination, abrogated cytotoxic activity in the mutant cells, whereas inhibition of both kinases was required to reduce wildtype NK activity. Colucci et al. (2002) concluded that intracellular signaling in the adaptive immune system, i.e., in B and T cells, is fundamentally different from that in the NK cells of the innate immune system.

It is appreciated that the abovementioned animal model for KLRC4 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Colucci, F.; Schweighoffer, E.; Tomasello, E.; Turner, M.; Ortaldo, J. R.; Vivier, E.; Tybulewicz, V. L. J.; Di Santo, J. P.: Natural cytotoxicity uncoupled from the Syk and ZAP-70 intracellular kinases. Nature Immun. 3: 288-294, 2002.; and
Groh, V.; Rhinehart, R.; Randolph-Habecker, J.; Topp, M. S.; Riddell, S. R.; Spies, T.: Costimulation of CD8-alpha-beta T cells by NKG2D via engagement by MIC induced on virus-infected.

Further studies establishing the function and utilities of KLRC4 are found in John Hopkins OMIM database record ID 602893, and in sited publications numbered 1801, 3055-1805, 600, 1806, 1852-57 and 602 listed in the bibliography section hereinbelow, which are also hereby incorporated by reference. Myeloid Leukemia Factor 2 (MLF2, Accession NM_{—}005439) is another VGAM68 host target gene. MLF2 BINDING SITE is HOST TARGET binding site found in the 3′ untranslated region of mRNA encoded by MLF2, corresponding to a HOST TARGET binding site such as BINDING SITE I, BINDING SITE II or BINDING SITE III. Table 2 illustrates the complementarity of the nucleotide sequences of MLF2 BINDING SITE, designated SEQ ID:1212, to the nucleotide sequence of VGAM68 RNA, herein designated VGAM RNA, also designated SEQ ID:403.

Another function of VGAM68 is therefore inhibition of Myeloid Leukemia Factor 2 (MLF2, Accession NM_{—}005439). Accordingly, utilities of VGAM68 include diagnosis, prevention and treatment of diseases and clinical conditions associated with MLF2. Nuclear Receptor Sub family 1, Group 1, Member 2 (NR1I2, Accession NM_{—}003889) is another VGAM68 host target gene. NR1I2 BINDING SITE1 and NR1I2 BINDING SITE2 are HOST TARGET binding sites found in untranslated regions of mRNA encoded by NR1I2, corresponding to HOST TARGET binding sites such as BINDING SITE I, BINDING SITE II or BINDING SITE III. Table 2 illustrates the complementarity of the nucleotide sequences of NR1I2 BINDING SITE1 and NR1I2 BINDING SITE2, designated SEQ ID: 1067 and SEQ ID: 1971 respectively, to the nucleotide sequence of VGAM68 RNA, herein designated VGAM RNA, also designated SEQ ID:403.

Another function of VGAM68 is therefore inhibition of Nuclear Receptor Sub family 1, Group 1, Member 2 (NR1I2, Accession NM_{—}003889), a gene which binds to a response element in the cyp3a4 gene promoter and activates its expression in response to a wide variety of endobiotics and xenobiotics. Accordingly, utilities of VGAM68 include diagnosis, prevention and treatment of diseases and clinical conditions associated with NR1I2. The function of NR1I2 has been established by previous studies. Lehmann et al. (1998) identified a nuclear receptor, termed PXR, that binds to the rifampicin/dexamethasone response element in the CYP3A4 (OMIM Ref. No. 124010) promoter as a heterodimer with the 9-cis retinoic acid receptor RXR (see OMIM Ref. No. 180245). The human PXR is related to the mouse Pxr1, which they had cloned and shown to be activated by dexamethasone, pregnenolone 16-alpha-carbonitrile (PCN), and other compounds known to induce expression of the CYP3A1 gene, the predominant form of CYP3A in rat liver and intestine. Lehmann et al. (1998) isolated PXR clones from a human liver cDNA library. Amino acid sequence comparison showed that human PXR shared 96% and 76% sequence identity with mouse Pxr1 in the DNA-binding and ligand-binding domains, respectively. Initiation of translation at a CUG initiation codon would yield a protein of 434 amino acids. Northern blot analysis detected most abundant expression in liver, colon, and small intestine; transcripts of 2.6, 4.3, and 5 kb were present in each of these tissues. Lehmann et al. (1998) provided several lines of evidence indicating that human PXR serves as a key transcriptional regulator of the CYP3A4 gene. Animal model experiments lend further support to the function of NR1I2. The induction of CYP3A enzymes is species-specific and believed to involve 1 or more cellular factors, or receptor-like xenosensors. Xie et al. (2000) identified one such factor as the nuclear receptor Pxr and its human homolog SXR. Xie et al. (2000) showed that targeted disruption of the mouse Pxr gene abolished induction of CYP3A by prototypic inducers such as dexamethasone or pregnenolone-16-alpha-carbonitrile. In Pxr-null mice carrying a transgene for an activated form of human SXR, there was constitutive up-regulation of CYP3A gene expression and enhanced protection against toxic xenobiotic compounds. Xie et al. (2000) demonstrated that species origin of the receptor, rather than the promoter structure of the CYP3A genes, dictates the species-specific pattern of CYP3A inducibility. Thus, they could generate ‘humanized’ transgenic mice that were responsive to human-specific inducers such as the antibiotic rifampicin. Xie et al. (2000) concluded that the SXR/Pxr genes encode the primary species-specific xenosensors that mediate the adaptive hepatic response, and may represent the critical biochemical mechanism of human xenoprotection.

It is appreciated that the abovementioned animal model for NR1I2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Lehmann, J. M.; McKee, D. D.; Watson, M. A.; Willson, T. M.; Moore, J. T.; Kliewer, S. A.: The human orphan nuclear receptor PXR is activated by compounds that regulate CYP3A4 gene expression and cause drug interactions. J. Clin. Invest. 102: 1016-1023, 1998.; and
Xie, W.; Barwick, J. L.; Downes, M.; Blumberg, B.; Simon, C. M.; Nelson, M. C.; Neuschwander-Tetri, B. A.; Brunt, E. M.; Guzelian, P. S.; Evans, R. M.: Humanized xenobiotic response in.

Further studies establishing the function and utilities of NR1I2 are found in John Hopkins OMIM database record ID 603065, and in sited publications numbered 1918-1919, 785, 884, 192 and 1936 listed in the bibliography section hereinbelow, which are also hereby incorporated by reference. FHX (Accession NM_{—}018416) is another VGAM68 host target gene. FHX BINDING SITE is HOST TARGET binding site found in the 3′ untranslated region of mRNA encoded by FHX, corresponding to a HOST TARGET binding site such as BINDING SITE I, BINDING SITE II or BINDING SITE III. Table 2 illustrates the complementarity of the nucleotide sequences of FHX BINDING SITE, designated SEQ ID: 1825, to the nucleotide sequence of VGAM68 RNA, herein designated VGAM RNA, also designated SEQ ID:403.

Another function of VGAM68 is therefore inhibition of FHX (Accession NM_{—}018416). Accordingly, utilities of VGAM68 include diagnosis, prevention and treatment of diseases and clinical conditions associated with FHX. FLJ10352 (Accession NM_{—}032142) is another VGAM68 host target gene. FLJ10352 BINDING SITE is HOST TARGET binding site found in the 3′ untranslated region of mRNA encoded by FLJ10352, corresponding to a HOST TARGET binding site such as BINDING SITE I, BINDING SITE II or BINDING SITE III. Table 2 illustrates the complementarity of the nucleotide sequences of FLJ10352 BINDING SITE, designated SEQ ID:2233, to the nucleotide sequence of VGAM68 RNA, herein designated VGAM RNA, also designated SEQ ID:403.

Another function of VGAM68 is therefore inhibition of FLJ10352 (Accession NM_{—}032142). Accordingly, utilities of VGAM68 include diagnosis, prevention and treatment of diseases and clinical conditions associated with FLJ10352. FLJ22127 (Accession NM_{—}022775) is another VGAM68 host target gene. FLJ22127 BINDING SITE is HOST TARGET binding site found in the 3′ untranslated region of mRNA encoded by FLJ22127, corresponding to a HOST TARGET binding site such as BINDING SITE I, BINDING SITE II or BINDING SITE III. Table 2 illustrates the complementarity of the nucleotide sequences of FLJ22127 BINDING SITE, designated SEQ ID:2008, to the nucleotide sequence of VGAM68 RNA, herein designated VGAM RNA, also designated SEQ ID:403.

Another function of VGAM68 is therefore inhibition of FLJ22127 (Accession NM_{—}022775). Accordingly, utilities of VGAM68 include diagnosis, prevention and treatment of diseases and clinical conditions associated with FLJ22127. Ganglioside-induced Differentiation-associated Protein 1-like 1 (GDAP1L1, Accession NM_{—}024034) is another VGAM68 host target gene. GDAP1L1 BINDING SITE is HOST TARGET binding site found in the 3′ untranslated region of mRNA encoded by GDAP1L1, corresponding to a HOST TARGET binding site such as BINDING SITE I, BINDING SITE II or BINDING SITE III. Table 2 illustrates the complementarity of the nucleotide sequences of GDAP1L1 BINDING SITE, designated SEQ ID:2043, to the nucleotide sequence of VGAM68 RNA, herein designated VGAM RNA, also designated SEQ ID:403.

Another function of VGAM68 is therefore inhibition of Ganglioside-induced Differentiation-associated Protein 1-like 1 (GDAP1L1, Accession NM_{—}024034). Accordingly, utilities of VGAM68 include diagnosis, prevention and treatment of diseases and clinical conditions associated with GDAP1L1. H-L(3)MBT (Accession NM_{—}032107) is another VGAM68 host target gene. H-L(3)MBT BINDING SITE is HOST TARGET binding site found in the 3′ untranslated region of mRNA encoded by H-L(3)MBT, corresponding to a HOST TARGET binding site such as BINDING SITE I, BINDING SITE II or BINDING SITE III. Table 2 illustrates the complementarity of the nucleotide sequences of HL(3)MBT BINDING SITE, designated SEQ ID:2230, to the nucleotide sequence of VGAM68 RNA, herein designated VGAM RNA, also designated SEQ ID:403.

Another function of VGAM68 is therefore inhibition of HL(3)MBT (Accession NM_{—}032107). Accordingly, utilities of VGAM68 include diagnosis, prevention and treatment of diseases and clinical conditions associated with HL(3)MBT. KIAA1340 (Accession XM_{—}044836) is another VGAM68 host target gene. KIAA1340 BINDING SITE is HOST TARGET binding site found in the 3′ untranslated region of mRNA encoded by KIAA1340, corresponding to a HOST TARGET binding site such as BINDING SITE I, BINDING SITE II or BINDING SITE III. Table 2 illustrates the complementarity of the nucleotide sequences of KIAA1340 BINDING SITE, designated SEQ ID:2846, to the nucleotide sequence of VGAM68 RNA, herein designated VGAM RNA, also designated SEQ ID:403.

Another function of VGAM68 is therefore inhibition of KIAA1340 (Accession XM_{—}044836). Accordingly, utilities of VGAM68 include diagnosis, prevention and treatment of diseases and clinical conditions associated with KIAA1340. Kinesin-like 7 (KNSL7, Accession NM_{—}020242) is another VGAM68 host target gene. KNSL7 BINDING SITE is HOST TARGET binding site found in the 3′ untranslated region of mRNA encoded by KNSL7, corresponding to a HOST TARGET binding site such as BINDING SITE I, BINDING SITE II or BINDING SITE III. Table 2 illustrates the complementarity of the nucleotide sequences of KNSL7 BINDING SITE, designated SEQ ID:1905, to the nucleotide sequence of VGAM68 RNA, herein designated VGAM RNA, also designated SEQ ID:403.

Another function of VGAM68 is therefore inhibition of Kinesin-like 7 (KNSL7, Accession NM_{—}020242). Accordingly, utilities of VGAM68 include diagnosis, prevention and treatment of diseases and clinical conditions associated with KNSL7. P5-1 (Accession NM_{—}006674) is another VGAM68 host target gene. P5-1 BINDING SITE is HOST TARGET binding site found in the 3′ untranslated region of mRNA encoded by P5-1, corresponding to a HOST TARGET binding site such as BINDING SITE I, BINDING SITE II or BINDING SITE III. Table 2 illustrates the complementarity of the nucleotide sequences of P5-1 BINDING SITE, designated SEQ ID:1322, to the nucleotide sequence of VGAM68 RNA, herein designated VGAM RNA, also designated SEQ ID:403.

Another function of VGAM68 is therefore inhibition of P5-1 (Accession NM_{—}006674). Accordingly, utilities of VGAM68 include diagnosis, prevention and treatment of diseases and clinical conditions associated with P5-1. PP1044 (Accession NM_{—}021730) is another VGAM68 host target gene. PP1044 BINDING SITE is HOST TARGET binding site found in the 5′ untranslated region of mRNA encoded by PP1044, corresponding to a HOST TARGET binding site such as BINDING SITE I, BINDING SITE II or BINDING SITE III. Table 2 illustrates the complementarity of the nucleotide sequences of PP1044 BINDING SITE, designated SEQ ID: 1956, to the nucleotide sequence of VGAM68 RNA, herein designated VGAM RNA, also designated SEQ ID:403.

Another function of VGAM68 is therefore inhibition of PP1044 (Accession NM_{—}021730). Accordingly, utilities of VGAM68 include diagnosis, prevention and treatment of diseases and clinical conditions associated with PP1044. Signal-regulatory Protein Beta 1 (SIRPB1, Accession NM_{—}006065) is another VGAM68 host target gene. SIRPB1 BINDING SITE is HOST TARGET binding site found in the 3′ untranslated region of mRNA encoded by SIRPB1, corresponding to a HOST TARGET binding site such as BINDING SITE I, BINDING SITE II or BINDING SITE III. Table 2 illustrates the complementarity of the nucleotide sequences of SIRPB1 BINDING SITE, designated SEQ ID:1272, to the nucleotide sequence of VGAM68 RNA, herein designated VGAM RNA, also designated SEQ ID:403.

Another function of VGAM68 is therefore inhibition of Signal-regulatory Protein Beta 1 (SIRPB1, Accession NM_{—}006065). Accordingly, utilities of VGAM68 include diagnosis, prevention and treatment of diseases and clinical conditions associated with SIRPB1. LOC148811 (Accession XM_{—}086326) is another VGAM68 host target gene. LOC148811 BINDING SITE is HOST TARGET binding site found in the 3′ untranslated region of mRNA encoded by LOC148811, corresponding to a HOST TARGET binding site such as BINDING SITE I, BINDING SITE II or BINDING SITE III. Table 2 illustrates the complementarity of the nucleotide sequences of LOC148811 BINDING SITE, designated SEQ ID:3126, to the nucleotide sequence of VGAM68 RNA, herein designated VGAM RNA, also designated SEQ ID:403.

Another function of VGAM68 is therefore inhibition of LOC148811 (Accession XM_{—}086326). Accordingly, utilities of VGAM68 include diagnosis, prevention and treatment of diseases and clinical conditions associated with LOC148811. LOC149711 (Accession XM_{—}097720) is another VGAM68 host target gene. LOC149711 BINDING SITE is HOST TARGET binding site found in the 3′ untranslated region of mRNA encoded by LOC149711, corresponding to a HOST TARGET binding site such as BINDING SITE I, BINDING SITE II or BINDING SITE III. Table 2 illustrates the complementarity of the nucleotide sequences of LOC149711 BINDING SITE, designated SEQ ID:3315, to the nucleotide sequence of VGAM68 RNA, herein designated VGAM RNA, also designated SEQ ID:403.

Another function of VGAM68 is therefore inhibition of LOC149711 (Accession XM_{—}097720). Accordingly, utilities of VGAM68 include diagnosis, prevention and treatment of diseases and clinical conditions associated with LOC149711. LOC150630 (Accession XM_{—}097931) is another VGAM68 host target gene. LOC150630 BINDING SITE is HOST TARGET binding site found in the 5′ untranslated region of mRNA encoded by LOC150630, corresponding to a HOST TARGET binding site such as BINDING SITE I, BINDING SITE II or BINDING SITE III. Table 2 illustrates the complementarity of the nucleotide sequences of LOC150630 BINDING SITE, designated SEQ ID:3332, to the nucleotide sequence of VGAM68 RNA, herein designated VGAM RNA, also designated SEQ ID:403.

Another function of VGAM68 is therefore inhibition of LOC150630 (Accession XM_{—}097931). Accordingly, utilities of VGAM68 include diagnosis, prevention and treatment of diseases and clinical conditions associated with LOC150630. LOC150819 (Accession XM_{—}097954) is another VGAM68 host target gene. LOC150819 BINDING SITE is HOST TARGET binding site found in the 5′ untranslated region of mRNA encoded by LOC150819, corresponding to a HOST TARGET binding site such as BINDING SITE I, BINDING SITE II or BINDING SITE III. Table 2 illustrates the complementarity of the nucleotide sequences of LOC150819 BINDING SITE, designated SEQ ID:3333, to the nucleotide sequence of VGAM68 RNA, herein designated VGAM RNA, also designated SEQ ID:403.

Another function of VGAM68 is therefore inhibition of LOC150819 (Accession XM_{—}097954). Accordingly, utilities of VGAM68 include diagnosis, prevention and treatment of diseases and clinical conditions associated with LOC150819. LOC154877 (Accession XM_{—}098626) is another VGAM68 host target gene. LOC154877 BINDING SITE is HOST TARGET binding site found in the 3′ untranslated region of mRNA encoded by LOC154877, corresponding to a HOST TARGET binding site such as BINDING SITE I, BINDING SITE II or BINDING SITE III. Table 2 illustrates the complementarity of the nucleotide sequences of LOC154877 BINDING SITE, designated SEQ ID:3373, to the nucleotide sequence of VGAM68 RNA, herein designated VGAM RNA, also designated SEQ ID:403.

Another function of VGAM68 is therefore inhibition of LOC154877 (Accession XM_{—}098626). Accordingly, utilities of VGAM68 include diagnosis, prevention and treatment of diseases and clinical conditions associated with LOC154877. LOC158327 (Accession XM_{—}088548) is another VGAM68 host target gene. LOC158327 BINDING SITE is HOST TARGET binding site found in the 3′ untranslated region of mRNA encoded by LOC158327, corresponding to a HOST TARGET binding site such as BINDING SITE I, BINDING SITE II or BINDING SITE III. Table 2 illustrates the complementarity of the nucleotide sequences of LOC158327 BINDING SITE, designated SEQ ID:3216, to the nucleotide sequence of VGAM68 RNA, herein designated VGAM RNA, also designated SEQ ID:403.

Another function of VGAM68 is therefore inhibition of LOC158327 (Accession XM_{—}088548). Accordingly, utilities of VGAM68 include diagnosis, prevention and treatment of diseases and clinical conditions associated with LOC158327. LOC196540 (Accession XM_{—}116933) is another VGAM68 host target gene. LOC196540 BINDING SITE is HOST TARGET binding site found in the 3′ untranslated region of mRNA encoded by LOC196540, corresponding to a HOST TARGET binding site such as BINDING SITE I, BINDING SITE II or BINDING SITE III. Table 2 illustrates the complementarity of the nucleotide sequences of LOC196540 BINDING SITE, designated SEQ ID:3467, to the nucleotide sequence of VGAM68 RNA, herein designated VGAM RNA, also designated SEQ ID:403.

Another function of VGAM68 is therefore inhibition of LOC196540 (Accession XM_{—}116933). Accordingly, utilities of VGAM68 include diagnosis, prevention and treatment of diseases and clinical conditions associated with LOC196540. LOC220394 (Accession XM_{—}166936) is another VGAM68 host target gene. LOC220394 BINDING SITE is HOST TARGET binding site found in the 5′ untranslated region of mRNA encoded by LOC220394, corresponding to a HOST TARGET binding site such as BINDING SITE I, BINDING SITE II or BINDING SITE III. Table 2 illustrates the complementarity of the nucleotide sequences of LOC220394 BINDING SITE, designated SEQ ID:3581, to the nucleotide sequence of VGAM68 RNA, herein designated VGAM RNA, also designated SEQ ID:403.

Another function of VGAM68 is therefore inhibition of LOC220394 (Accession XM_{—}166936). Accordingly, utilities of VGAM68 include diagnosis, prevention and treatment of diseases and clinical conditions associated with LOC220394. LOC254428 (Accession XM_{—}170932) is another VGAM68 host target gene. LOC254428 BINDING SITE is HOST TARGET binding site found in the 3′ untranslated region of mRNA encoded by LOC254428, corresponding to a HOST TARGET binding site such as BINDING SITE I, BINDING SITE II or BINDING SITE III. Table 2 illustrates the complementarity of the nucleotide sequences of LOC254428 BINDING SITE, designated SEQ ID:3677, to the nucleotide sequence of VGAM68 RNA, herein designated VGAM RNA, also designated SEQ ID:403.

Another function of VGAM68 is therefore inhibition of LOC254428 (Accession XM_{—}170932). Accordingly, utilities of VGAM68 include diagnosis, prevention and treatment of diseases and clinical conditions associated with LOC254428. LOC92405 (Accession XM_{—}044914) is another VGAM68 host target gene. LOC92405 BINDING SITE is HOST TARGET binding site found in the 3′ untranslated region of mRNA encoded by LOC92405, corresponding to a HOST TARGET binding site such as BINDING SITE I, BINDING SITE II or BINDING SITE III. Table 2 illustrates the complementarity of the nucleotide sequences of LOC92405 BINDING SITE, designated SEQ ID:2848, to the nucleotide sequence of VGAM68 RNA, herein designated VGAM RNA, also designated SEQ ID:403.

Another function of VGAM68 is therefore inhibition of LOC92405 (Accession XM_{—}044914). Accordingly, utilities of VGAM68 include diagnosis, prevention and treatment of diseases and clinical conditions associated with LOC92405. FIG. 1 further provides a conceptual description of a novel bioinformatically detected viral gene of the present invention, referred to here as Viral Genomic Address Messenger 69 (VGAM69) viral gene, which modulates expression of respective host target genes thereof, the function and utility of which host target genes is known in the art.

VGAM69 is a novel bioinformatically detected regulatory, non protein coding, viral micro RNA (miRNA) gene. The method by which VGAM69 was detected is described hereinabove with reference to FIGS. 1-8.

VGAM69 gene, herein designated VGAM GENE, is a viral gene contained in the genome of Vaccinia Virus. VGAM69 host target gene, herein designated VGAM HOST TARGET GENE, is a human gene contained in the human genome.

VGAM69 gene encodes a VGAM69 precursor RNA, herein designated VGAM PRECURSOR RNA. Similar to other miRNA genes, and unlike most ordinary genes, VGAM69 precursor RNA does not encode a protein. A nucleotide sequence identical or highly similar to the nucleotide sequence of VGAM69 precursor RNA is designated SEQ ID: 55, and is provided hereinbelow with reference to the sequence listing part. Nucleotide sequence SEQ ID:55 is located at position 180452 relative to the genome of Vaccinia Virus.

VGAM69 precursor RNA folds onto itself, forming VGAM69 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM69 folded precursor RNA into VGAM69 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM69 RNA is designated SEQ ID:404, and is provided hereinbelow with reference to the sequence listing part.

VGAM69 host target gene, herein designated VGAM HOST TARGET GENE, encodes a corresponding messenger RNA, VGAM69 host target RNA, herein designated VGAM HOST TARGET RNA. VGAM69 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM69 RNA, herein designated VGAM RNA, binds complementarily to one or more host target binding sites located in untranslated regions of VGAM69 host target RNA, herein designated VGAM HOST TARGET RNA. This complementary binding is due to the fact that the nucleotide sequence of VGAM69 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM69 RNA, herein designated VGAM RNA, may have a different number of host target binding sites in untranslated regions of a VGAM69 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM69 RNA, herein designated VGAM RNA, to host target binding sites on VGAM69 host target RNA, herein designated VGAM HOST TARGET RNA, such as BINDING SITE I, BINDING SITE II and BINDING SITE III, inhibits translation of VGAM69 host target RNA into VGAM69 host target protein, herein designated VGAM HOST TARGET PROTEIN. VGAM host target protein is therefore outlined by a broken line.

It is appreciated that VGAM69 host target gene, herein designated VGAM HOST TARGET GENE, in fact represents a plurality of VGAM69 host target genes. The mRNA of each one of this plurality of VGAM69 host target genes comprises one or more host target binding sites, each having a nucleotide sequence which is at least partly complementary to VGAM69 RNA, herein designated VGAM RNA, and which when bound by VGAM69 RNA causes inhibition of translation of respective one or more VGAM69 host target proteins.

It is further appreciated by one skilled in the art that the mode of translational inhibition illustrated by FIG. 1 with specific reference to translational inhibition exerted by VGAM69 gene, herein designated VGAM GENE, on one or more VGAM69 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM69 is inhibition of expression of host target genes, as part of a novel viral mechanism of attacking a host. Accordingly, utilities of VGAM69 include diagnosis, prevention and treatment of viral infection by Vaccinia Virus. Specific functions, and accordingly utilities, of VGAM69 correlate with, and may be deduced from, the identity of the host target genes which VGAM69 binds and inhibits, and the function of these host target genes, as elaborated hereinbelow.

Nucleotide sequences of the VGAM69 precursor RNA, herein designated VGAM PRECURSOR RNA, and of the ‘diced’ VGAM69 RNA, herein designated VGAM RNA, and a schematic representation of the secondary folding of VGAM69 folded precursor RNA, herein designated VGAM FOLDED PRECURSOR RNA, of VGAM69 are further described hereinbelow with reference to Table 1.

Nucleotide sequences of host target binding sites, such as BINDING SITE-I, BINDING SITE-II and BINDING SITE-III of FIG. 1, found on VGAM69 host target RNA, and schematic representation of the complementarity of each of these host target binding sites to VGAM69 RNA, herein designated VGAM RNA, are described hereinbelow with reference to Table 2.

As mentioned hereinabove with reference to FIG. 1, a function of VGAM69 gene, herein designated VGAM is inhibition of expression of VGAM69 target genes. It is appreciated that specific functions, and accordingly utilities, of VGAM69 correlate with, and may be deduced from, the identity of the target genes which VGAM69 binds and inhibits, and the function of these target genes, as elaborated hereinbelow.

DKFZP434G1411 (Accession XM_{—}166383) is a VGAM69 host target gene. DKFZP434G1411 BINDING SITE is HOST TARGET binding site found in the 3′ untranslated region of mRNA encoded by DKFZP434G1411, corresponding to a HOST TARGET binding site such as BINDING SITE I, BINDING SITE II or BINDING SITE III. Table 2 illustrates the complementarity of the nucleotide sequences of DKFZP434G1411 BINDING SITE, designated SEQ ID:3554, to the nucleotide sequence of VGAM69 RNA, herein designated VGAM RNA, also designated SEQ ID:404.

A function of VGAM69 is therefore inhibition of DKFZP434G1411 (Accession XM_{—}166383). Accordingly, utilities of VGAM69 include diagnosis, prevention and treatment of diseases and clinical conditions associated with DKFZP434G1411. DKFZP5641052 (Accession XM_{—}039660) is another VGAM69 host target gene. DKFZP5641052 BINDING SITE is HOST TARGET binding site found in the 3′ untranslated region of mRNA encoded by DKFZP5641052, corresponding to a HOST TARGET binding site such as BINDING SITE I, BINDING SITE II or BINDING SITE III. Table 2 illustrates the complementarity of the nucleotide sequences of DKFZP5641052 BINDING SITE, designated SEQ ID:2760, to the nucleotide sequence of VGAM69 RNA, herein designated VGAM RNA, also designated SEQ ID:404.

Another function of VGAM69 is therefore inhibition of DKFZP5641052 (Accession XM_{—}039660). Accordingly, utilities of VGAM69 include diagnosis, prevention and treatment of diseases and clinical conditions associated with DKFZP5641052. Kelch-like 8 (Drosophila) (KLHL8, Accession XM_{—}031735) is another VGAM69 host target gene. KLHL8 BINDING SITE is HOST TARGET binding site found in the 3′ untranslated region of mRNA encoded by KLHL8, corresponding to a HOST TARGET binding site such as BINDING SITE I, BINDING SITE II or BINDING SITE III. Table 2 illustrates the complementarity of the nucleotide sequences of KLHL8 BINDING SITE, designated SEQ ID:2639, to the nucleotide sequence of VGAM69 RNA, herein designated VGAM RNA, also designated SEQ ID:404.

Another function of VGAM69 is therefore inhibition of Kelch-like 8 (Drosophila) (KLHL8, Accession XM_{—}031735). Accordingly, utilities of VGAM69 include diagnosis, prevention and treatment of diseases and clinical conditions associated with KLHL8. Extracellular Link Domain Containing 1 (XLKD1, Accession NM_{—}006691) is another VGAM69 host target gene. XLKD1 BINDING SITE is HOST TARGET binding site found in the 3′ untranslated region of mRNA encoded by XLKD1, corresponding to a HOST TARGET binding site such as BINDING SITE I, BINDING SITE II or BINDING SITE III. Table 2 illustrates the complementarity of the nucleotide sequences of XLKD1 BINDING SITE, designated SEQ ID:1323, to the nucleotide sequence of VGAM69 RNA, herein designated VGAM RNA, also designated SEQ ID:404.

Another function of VGAM69 is therefore inhibition of Extracellular Link Domain Containing 1 (XLKD1, Accession NM_{—}006691). Accordingly, utilities of VGAM69 include diagnosis, prevention and treatment of diseases and clinical conditions associated with XLKD1. LOC51003 (Accession NM_{—}016060) is another VGAM69 host target gene. LOC51003 BINDING SITE is HOST TARGET binding site found in the 3′ untranslated region of mRNA encoded by LOC51003, corresponding to a HOST TARGET binding site such as BINDING SITE I, BINDING SITE II or BINDING SITE III. Table 2 illustrates the complementarity of the nucleotide sequences of LOC51003 BINDING SITE, designated SEQ ID: 1658, to the nucleotide sequence of VGAM69 RNA, herein designated VGAM RNA, also designated SEQ ID:404.

Another function of VGAM69 is therefore inhibition of LOC51003 (Accession NM_{—}016060). Accordingly, utilities of VGAM69 include diagnosis, prevention and treatment of diseases and clinical conditions associated with LOC51003. FIG. 1 further provides a conceptual description of a novel bioinformatically detected viral gene of the present invention, referred to here as Viral Genomic Address Messenger 70 (VGAM70) viral gene, which modulates expression of respective host target genes thereof, the function and utility of which host target genes is known in the art.

VGAM70 is a novel bioinformatically detected regulatory, non protein coding, viral micro RNA (miRNA) gene. The method by which VGAM70 was detected is described hereinabove with reference to FIGS. 1-8.

VGAM70 gene, herein designated VGAM GENE, is a viral gene contained in the genome of Vaccinia Virus. VGAM70 host target gene, herein designated VGAM HOST TARGET GENE, is a human gene contained in the human genome.

VGAM70 gene encodes a VGAM70 precursor RNA, herein designated VGAM PRECURSOR RNA. Similar to other miRNA genes, and unlike most ordinary genes, VGAM70 precursor RNA does not encode a protein. A nucleotide sequence identical or highly similar to the nucleotide sequence of VGAM70 precursor RNA is designated SEQ ID:56, and is provided hereinbelow with reference to the sequence listing part. Nucleotide sequence SEQ ID:56 is located at position 12513 relative to the genome of Vaccinia Virus.

VGAM70 precursor RNA folds onto itself, forming VGAM70 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM70 folded precursor RNA into VGAM70 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM70 RNA is designated SEQ ID:405, and is provided hereinbelow with reference to the sequence listing part.

VGAM70 host target gene, herein designated VGAM HOST TARGET GENE, encodes a corresponding messenger RNA, VGAM70 host target RNA, herein designated VGAM HOST TARGET RNA. VGAM70 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM70 RNA, herein designated VGAM RNA, binds complementarily to one or more host target binding sites located in untranslated regions of VGAM70 host target RNA, herein designated VGAM HOST TARGET RNA. This complementary binding is due to the fact that the nucleotide sequence of VGAM70 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM70 RNA, herein designated VGAM RNA, may have a different number of host target binding sites in untranslated regions of a VGAM70 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM70 RNA, herein designated VGAM RNA, to host target binding sites on VGAM70 host target RNA, herein designated VGAM HOST TARGET RNA, such as BINDING SITE I, BINDING SITE II and BINDING SITE III, inhibits translation of VGAM70 host target RNA into VGAM70 host target protein, herein designated VGAM HOST TARGET PROTEIN. VGAM host target protein is therefore outlined by a broken line.

It is appreciated that VGAM70 host target gene, herein designated VGAM HOST TARGET GENE, in fact represents a plurality of VGAM70 host target genes. The mRNA of each one of this plurality of VGAM70 host target genes comprises one or more host target binding sites, each having a nucleotide sequence which is at least partly complementary to VGAM70 RNA, herein designated VGAM RNA, and which when bound by VGAM70 RNA causes inhibition of translation of respective one or more VGAM70 host target proteins.

It is further appreciated by one skilled in the art that the mode of translational inhibition illustrated by FIG. 1 with specific reference to translational inhibition exerted by VGAM70 gene, herein designated VGAM GENE, on one or more VGAM70 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM70 is inhibition of expression of host target genes, as part of a novel viral mechanism of attacking a host. Accordingly, utilities of VGAM70 include diagnosis, prevention and treatment of viral infection by Vaccinia Virus. Specific functions, and accordingly utilities, of VGAM70 correlate with, and may be deduced from, the identity of the host target genes which VGAM70 binds and inhibits, and the function of these host target genes, as elaborated hereinbelow.

Nucleotide sequences of the VGAM70 precursor RNA, herein designated VGAM PRECURSOR RNA, and of the ‘diced’ VGAM70 RNA, herein designated VGAM RNA, and a schematic representation of the secondary folding of VGAM70 folded precursor RNA, herein designated VGAM FOLDED PRECURSOR RNA, of VGAM70 are further described hereinbelow with reference to Table 1.

Nucleotide sequences of host target binding sites, such as BINDING SITE-I, BINDING SITE-II and BINDING SITE-III of FIG. 1, found on VGAM70 host target RNA, and schematic representation of the complementarity of each of these host target binding sites to VGAM70 RNA, herein designated VGAM RNA, are described hereinbelow with reference to Table 2.

As mentioned hereinabove with reference to FIG. 1, a function of VGAM70 gene, herein designated VGAM is inhibition of expression of VGAM70 target genes. It is appreciated that specific functions, and accordingly utilities, of VGAM70 correlate with, and may be deduced from, the identity of the target genes which VGAM70 binds and inhibits, and the function of these target genes, as elaborated hereinbelow.

Sialidase 3 (membrane sialidase) (NEU3, Accession NM_{—}006656) is a VGAM70 host target gene. NEU3 BINDING SITE is HOST TARGET binding site found in the 3′ untranslated region of mRNA encoded by NEU3, corresponding to a HOST TARGET binding site such as BINDING SITE I, BINDING SITE II or BINDING SITE III. Table 2 illustrates the complementarity of the nucleotide sequences of NEU3 BINDING SITE, designated SEQ ID:1319, to the nucleotide sequence of VGAM70 RNA, herein designated VGAM RNA, also designated SEQ ID:405.

A function of VGAM70 is therefore inhibition of Sialidase 3 (membrane sialidase) (NEU3, Accession NM_{—}006656). Accordingly, utilities of VGAM70 include diagnosis, prevention and treatment of diseases and clinical conditions associated with NEU3. Choline Phosphotransferase 1 (CHPT1, Accession NM_{—}020244) is another VGAM70 host target gene. CHPT1 BINDING SITE is HOST TARGET binding site found in the 3′ untranslated region of mRNA encoded by CHPT1, corresponding to a HOST TARGET binding site such as BINDING SITE I, BINDING SITE II or BINDING SITE III. Table 2 illustrates the complementarity of the nucleotide sequences of CHPT1 BINDING SITE, designated SEQ ID:1906, to the nucleotide sequence of VGAM70 RNA, herein designated VGAM RNA, also designated SEQ ID:405.

Another function of VGAM70 is therefore inhibition of Choline Phosphotransferase 1 (CHPT1, Accession NM_{—}020244). Accordingly, utilities of VGAM70 include diagnosis, prevention and treatment of diseases and clinical conditions associated with CHPT1. LOC143310 (Accession XM_{—}084485) is another VGAM70 host target gene. LOC143310 BINDING SITE is HOST TARGET binding site found in the 3′ untranslated region of mRNA encoded by LOC143310, corresponding to a HOST TARGET binding site such as BINDING SITE I, BINDING SITE II or BINDING SITE III. Table 2 illustrates the complementarity of the nucleotide sequences of LOC143310 BINDING SITE, designated SEQ ID:3057, to the nucleotide sequence of VGAM70 RNA, herein designated VGAM RNA, also designated SEQ ID:405.

Another function of VGAM70 is therefore inhibition of LOC143310 (Accession XM_{—}084485). Accordingly, utilities of VGAM70 include diagnosis, prevention and treatment of diseases and clinical conditions associated with LOC143310. LOC152578 (Accession XM_{—}098241) is another VGAM70 host target gene. LOC152578 BINDING SITE is HOST TARGET binding site found in the 5′ untranslated region of mRNA encoded by LOC152578, corresponding to a HOST TARGET binding site such as BINDING SITE I, BINDING SITE II or BINDING SITE III. Table 2 illustrates the complementarity of the nucleotide sequences of LOC152578 BINDING SITE, designated SEQ ID:3361, to the nucleotide sequence of VGAM70 RNA, herein designated VGAM RNA, also designated SEQ ID:405.

Another function of VGAM70 is therefore inhibition of LOC152578 (Accession XM_{—}098241). Accordingly, utilities of VGAM70 include diagnosis, prevention and treatment of diseases and clinical conditions associated with LOC152578. FIG. 1 further provides a conceptual description of a novel bioinformatically detected viral gene of the present invention, referred to here as Viral Genomic Address Messenger 71 (VGAM71) viral gene, which modulates expression of respective host target genes thereof, the function and utility of which host target genes is known in the art.

VGAM71 is a novel bioinformatically detected regulatory, non protein coding, viral micro RNA (miRNA) gene. The method by which VGAM71 was detected is described hereinabove with reference to FIGS. 1-8.

VGAM71 gene, herein designated VGAM GENE, is a viral gene contained in the genome of Vaccinia Virus. VGAM71 host target gene, herein designated VGAM HOST TARGET GENE, is a human gene contained in the human genome.

VGAM71 gene encodes a VGAM71 precursor RNA, herein designated VGAM PRECURSOR RNA. Similar to other miRNA genes, and unlike most ordinary genes, VGAM71 precursor RNA does not encode a protein. A nucleotide sequence identical or highly similar to the nucleotide sequence of VGAM71 precursor RNA is designated SEQ ID:57, and is provided hereinbelow with reference to the sequence listing part. Nucleotide sequence SEQ ID:57 is located at position 14150 relative to the genome of Vaccinia Virus.

VGAM71 precursor RNA folds onto itself, forming VGAM71 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM71 folded precursor RNA into VGAM71 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM71 RNA is designated SEQ ID:406, and is provided hereinbelow with reference to the sequence listing part.

VGAM71 host target gene, herein designated VGAM HOST TARGET GENE, encodes a corresponding messenger RNA, VGAM71 host target RNA, herein designated VGAM HOST TARGET RNA. VGAM71 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM71 RNA, herein designated VGAM RNA, binds complementarily to one or more host target binding sites located in untranslated regions of VGAM71 host target RNA, herein designated VGAM HOST TARGET RNA. This complementary binding is due to the fact that the nucleotide sequence of VGAM71 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM71 RNA, herein designated VGAM RNA, may have a different number of host target binding sites in untranslated regions of a VGAM71 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM71 RNA, herein designated VGAM RNA, to host target binding sites on VGAM71 host target RNA, herein designated VGAM HOST TARGET RNA, such as BINDING SITE I, BINDING SITE II and BINDING SITE III, inhibits translation of VGAM71 host target RNA into VGAM71 host target protein, herein designated VGAM HOST TARGET PROTEIN. VGAM host target protein is therefore outlined by a broken line.

It is appreciated that VGAM71 host target gene, herein designated VGAM HOST TARGET GENE, in fact represents a plurality of VGAM71 host target genes. The mRNA of each one of this plurality of VGAM71 host target genes comprises one or more host target binding sites, each having a nucleotide sequence which is at least partly complementary to VGAM71 RNA, herein designated VGAM RNA, and which when bound by VGAM71 RNA causes inhibition of translation of respective one or more VGAM71 host target proteins.

It is further appreciated by one skilled in the art that the mode of translational inhibition illustrated by FIG. 1 with specific reference to translational inhibition exerted by VGAM71 gene, herein designated VGAM GENE, on one or more VGAM71 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM71 is inhibition of expression of host target genes, as part of a novel viral mechanism of attacking a host. Accordingly, utilities of VGAM71 include diagnosis, prevention and treatment of viral infection by Vaccinia Virus. Specific functions, and accordingly utilities, of VGAM71 correlate with, and may be deduced from, the identity of the host target genes which VGAM71 binds and inhibits, and the function of these host target genes, as elaborated hereinbelow.

Nucleotide sequences of the VGAM71 precursor RNA, herein designated VGAM PRECURSOR RNA, and of the ‘diced’ VGAM71 RNA, herein designated VGAM RNA, and a schematic representation of the secondary folding of VGAM71 folded precursor RNA, herein designated VGAM FOLDED PRECURSOR RNA, of VGAM71 are further described hereinbelow with reference to Table 1.

Nucleotide sequences of host target binding sites, such as BINDING SITE-I, BINDING SITE-II and BINDING SITE-III of FIG. 1, found on VGAM71 host target RNA, and schematic representation of the complementarity of each of these host target binding sites to VGAM71 RNA, herein designated VGAM RNA, are described hereinbelow with reference to Table 2.

As mentioned hereinabove with reference to FIG. 1, a function of VGAM71 gene, herein designated VGAM is inhibition of expression of VGAM71 target genes. It is appreciated that specific functions, and accordingly utilities, of VGAM71 correlate with, and may be deduced from, the identity of the target genes which VGAM71 binds and inhibits, and the function of these target genes, as elaborated hereinbelow.

HNF3A (Accession XM_{—}007360) is a VGAM71 host target gene. HNF3A BINDING SITE is HOST TARGET binding site found in the 3′ untranslated region of mRNA encoded by HNF3A, corresponding to a HOST TARGET binding site such as BINDING SITE I, BINDING SITE II or BINDING SITE III. Table 2 illustrates the complementarity of the nucleotide sequences of HNF3A BINDING SITE, designated SEQ ID:2543, to the nucleotide sequence of VGAM71 RNA, herein designated VGAM RNA, also designated SEQ ID:406.

A function of VGAM71 is therefore inhibition of HNF3A (Accession XM_{—}007360), a gene which activates transcription for a number of liver genes. Accordingly, utilities of VGAM71 include diagnosis, prevention and treatment of diseases and clinical conditions associated with HNF3A. The function of HNF3A has been established by previous studies. The transcription factors Hnf3a and Gata4 (OMIM Ref. No. 600576) are the earliest known to bind the albumin gene enhancer in liver precursor cells in mouse embryos. To determine how they access sites in silent chromatin, Cirillo et al. (2002) assembled nucleosome arrays containing albumin enhancer sequences and compacted them with linker histone. Hnf3a and Gata4, but not human NF1 (see OMIM Ref. No. 600727), mouse Cebp-beta (OMIM Ref. No. 189965), or yeast GAL4-AH, bound their sites in compacted chromatin and opened the local nucleosomal domain in the absence of ATP-dependent enzymes. The authors showed that the ability of Hnf3a to open chromatin is mediated by a high-affinity DNA-binding site and by the C-terminal domain of the protein, which binds histones H3 and H4. They concluded that factors that potentiate transcription in development are inherently capable of initiating chromatin opening events. Mincheva et al. (1997) used fluorescence in situ hybridization to map HNF3A to human chromosome 14q12-q13. This chromosomal region contains a cluster of forkhead domain transcription factors including FKHL1 (OMIM Ref. No. 164874) and FKHL2 (OMIM Ref. No. 600779). By analysis of RFLPs in interspecific backcross mice, Avraham et al. (1992) mapped the mouse Hnf3-alpha gene to chromosome 12.

Full details of the abovementioned studies are described in the following publications, the disclosure of which are hereby incorporated by reference:
Cirillo, L. A.; Lin, F. R.; Cuesta, I.; Friedman, D.; Jarnik, M.; Zaret, K. S.: Opening of compacted chromatin by early developmental transcription factors HNF3(FoxA) and GATA-4. Molec. Cell 9: 279-289, 2002.; and
Mincheva, A.; Lichter, P.; Schutz, G.; Kaestner, K. H.: Assignment of the human genes for hepatocyte nuclear factor 3-alpha, -beta, and -gamma (HNF3A, HNF3B, HNF3G) to 14q12-q13, 20p11, a.

Further studies establishing the function and utilities of HNF3A are found in John Hopkins OMIM database record ID 602294, and in sited publications numbered 2289, 222 and 2290-2291 listed in the bibliography section hereinbelow, which are also hereby incorporated by reference. Mucin 12 (MUC12, Accession XM_{—}168546) is another VGAM71 host target gene. MUC12 BINDING SITE is HOST TARGET binding site found in the 3′ untranslated region of mRNA encoded by MUC12, corresponding to a HOST TARGET binding site such as BINDING SITE I, BINDING SITE II or BINDING SITE III. Table 2 illustrates the complementarity of the nucleotide sequences of MUC12 BINDING SITE, designated SEQ ID:3643, to the nucleotide sequence of VGAM71 RNA, herein designated VGAM RNA, also designated SEQ ID:406.

Another function of VGAM71 is therefore inhibition of Mucin 12 (MUC12, Accession XM_{—}168546). Accordingly, utilities of VGAM71 include diagnosis, prevention and treatment of diseases and clinical conditions associated with MUC12. Osteoglycin (osteoinductive factor, mimecan) (OGN, Accession NM_{—}033014) is another VGAM71 host target gene. OGN BINDING SITE1 through OGN BINDING SITE3 are HOST TARGET binding sites found in untranslated regions of mRNA encoded by OGN, corresponding to HOST TARGET binding sites such as BINDING SITE I, BINDING SITE II or BINDING SITE III. Table 2 illustrates the complementarity of the nucleotide sequences of OGN BINDING SITE1 through OGN BINDING SITE3, designated SEQ ID:2307, SEQ ID: 1467 and SEQ ID:2059 respectively, to the nucleotide sequence of VGAM71 RNA, herein designated VGAM RNA, also designated SEQ ID:406.

Another function of VGAM71 is therefore inhibition of Osteoglycin (osteoinductive factor, mimecan) (OGN, Accession NM_{—}033014), a gene which induces ectopic bone formation in conjunction with transforming growth factor beta. Accordingly, utilities of VGAM71 include diagnosis, prevention and treatment of diseases and clinical conditions associated with OGN. The function of OGN has been established by previous studies. Bovine osteoinductive factor (OIF) induces ectopic bone formation in conjunction with TGFB1 (OMIM Ref. No. 190180) or TGFB2 (OMIM Ref. No. 190220) (Bentz et al., 1989). Using primers based on the sequence of purified bovine OIF, Madisen et al. (1990) isolated a human OIF cDNA clone by RT-PCR of osteosarcoma cell mRNA. The human gene encodes a predicted 298-amino acid precursor protein that is processed into a 103-amino acid mature protein with 96% identity to the bovine protein. On Northern blots, 3 OIF mRNAs are found exclusively in 2 human osteosarcoma cell lines. By FISH, Tasheva et al. (2000) mapped the mimecan gene to 9q22. Pellegata et al. (2000) cloned the human OGN gene and mapped it to a region approximately 1.1 Mb telomeric of WI-532 and approximately 700 kb centromeric of D9S197 in 9q22.31.

Full details of the abovementioned studies are described in the following publications, the disclosure of which are hereby incorporated by reference:
Madisen, L.; Neubauer, M.; Plowman, G.; Rosen, D.; Segarini, P.; Dasch, J.; Thompson, A.; Ziman, J.; Bentz, H.; Purchio, A. F.: Molecular cloning of a novel bone-forming compound: osteoinductive factor. DNA Cell Biol. 9: 303-309, 1990.; and
Pellegata, N. S.; Dieguez-Lucena, J. L.; Joensuu, T.; Lau, S.; Montgomery, K. T.; Krahe, R.; Kivela, T.; Kucherlapati, R.; Forsius, H.; de la Chapelle, A.: Mutations in KERA, encoding.

Further studies establishing the function and utilities of OGN are found in John Hopkins OMIM database record ID 602383, and in sited publications numbered 1293-1294, 78 and 1346 listed in the bibliography section hereinbelow, which are also hereby incorporated by reference. Protein Tyrosine Phosphatase, Receptor Type, A (PTPRA, Accession NM_{—}080840) is another VGAM71 host target gene. PTPRA BINDING SITE1 through PTPRA BINDING SITE3 are HOST TARGET binding sites found in untranslated regions of mRNA encoded by PTPRA, corresponding to HOST TARGET binding sites such as BINDING SITE I, BINDING SITE II or BINDING SITE III. Table 2 illustrates the complementarity of the nucleotide sequences of PTPRA BINDING SITE1 through PTPRA BINDING SITE3, designated SEQ ID:2389, SEQ ID:2390 and SEQ ID:2344 respectively, to the nucleotide sequence of VGAM71 RNA, herein designated VGAM RNA, also designated SEQ ID:406.

Another function of VGAM71 is therefore inhibition of Protein Tyrosine Phosphatase, Receptor Type, A (PTPRA, Accession NM_{—}080840), a gene which is the human homolog of the murine PTPase. Accordingly, utilities of VGAM71 include diagnosis, prevention and treatment of diseases and clinical conditions associated with PTPRA. The function of PTPRA has been established by previous studies. Vital cellular functions such as cell proliferation and signal transduction are regulated in part by the balance between the activities of protein-tyrosine kinases (PTK) and protein-tyrosine phosphatases (OMIM Ref. No. PTPase). Oncogenesis can result from an imbalance. There are 2 classes of PTPase molecules: low molecular weight proteins with a single conserved phosphatase domain such as T-cell protein-tyrosine phosphatase (PTPT; 176887), and high molecular weight receptor-linked PT-Pases with 2 tandemly repeated conserved domains separated by 56 to 57 amino acids. Examples of the latter group include leukocyte-common antigen (PTPRC; 151460) and leukocyte antigen related tyrosine phosphatase (PTPRF; 179590). Matthews et al. (1990) cloned the human homolog of the murine PTPase termed LRP by them. Its cDNA sequence predicted a protein of 793 amino acids with an unglycosylated molecular mass of 87,500 kD Matthews et al. (1990). The protein contains a 121-residue extracellular domain, a single transmembrane segment, and 2 tandem intracytoplasmic catalytic domains. By study of rodent-human somatic cell hybrids, Jirik et al. (1990) localized PTPA/LRP to chromosome 20p13. Other family members located on chromosome 20 include SRC (OMIM Ref. No. 190090), HCK (OMIM Ref. No. 142370), and PTP1B (OMIM Ref. No. 176885). The LRP protein is ubiquitously expressed and thus likely plays a fundamental role in the physiology of all cells. With a leukocyte common antigen (LCA) probe, Kaplan et al. (1990) sequenced cDNA encoding the alpha enzyme isolated from a human brain stem cDNA library under conditions of reduced hybridization stringency. LRP encodes an 802-amino acid polypeptide. Kaplan et al. (1990) localized the RPTPase-alpha gene to human chromosome 20pter-20q12 by analysis of its segregation pattern in rodent-human somatic cell hybrids. Rao et al. (1992) regionalized the assignment of PTPA to the distal portion of 20p (20pter-p12) by both radioactive and fluorescence in situ hybridization. By in situ hybridization, Jirik et al. (1992) localized the PTPA gene to 20p13. With the mapping of PAX1 167411 to mouse chromosome 2, Schnittger et al. (1992) found that the homolog of PTPA is also located on mouse chromosome 2, which confirms the exceptional homology between human chromosome 20 and the distal segment of mouse chromosome 2.

Full details of the abovementioned studies are described in the following publications, the disclosure of which are hereby incorporated by reference:
Matthews, R. J.; Cahir, E. D.; Thomas, M. L.: Identification of an additional member of the protein-tyrosine-phosphatase family: evidence for alternative splicing in the tyrosine phosphatase domain. Proc. Nat. Acad. Sci. 87: 4444-4448, 1990.; and
Schnittger, S.; Rao, V. V. N. G.; Deutsch, U.; Gruss, P.; Balling, R.; Hansmann, I.: PAX1, a member of the paired box-containing class of developmental control genes, is mapped to human ch.

Further studies establishing the function and utilities of PTPRA are found in John Hopkins OMIM database record ID 176884, and in sited publications numbered 2500-2501, 249 and 2502-2504 listed in the bibliography section hereinbelow, which are also hereby incorporated by reference. FLJ22457 (Accession NM_{—}024901) is another VGAM71 host target gene. FLJ22457 BINDING SITE is HOST TARGET binding site found in the 3′ untranslated region of mRNA encoded by FLJ22457, corresponding to a HOST TARGET binding site such as BINDING SITE I, BINDING SITE II or BINDING SITE III. Table 2 illustrates the complementarity of the nucleotide sequences of FLJ22457 BINDING SITE, designated SEQ ID:2106, to the nucleotide sequence of VGAM71 RNA, herein designated VGAM RNA, also designated SEQ ID:406.

Another function of VGAM71 is therefore inhibition of FLJ22457 (Accession NM_{—}024901). Accordingly, utilities of VGAM71 include diagnosis, prevention and treatment of diseases and clinical conditions associated with FLJ22457. Lipase, Endothelial (LIPG, Accession NM_{—}006033) is another VGAM71 host target gene. LIPG BINDING SITE is HOST TARGET binding site found in the 3′ untranslated region of mRNA encoded by LIPG, corresponding to a HOST TARGET binding site such as BINDING SITE I, BINDING SITE II or BINDING SITE III. Table 2 illustrates the complementarity of the nucleotide sequences of LIPG BINDING SITE, designated SEQ ID:1266, to the nucleotide sequence of VGAM71 RNA, herein designated VGAM RNA, also designated SEQ ID:406.

Another function of VGAM71 is therefore inhibition of Lipase, Endothelial (LIPG, Accession NM_{—}006033). Accordingly, utilities of VGAM71 include diagnosis, prevention and treatment of diseases and clinical conditions associated with LIPG. MGC4643 (Accession NM_{—}032715) is another VGAM71 host target gene. MGC4643 BINDING SITE is HOST TARGET binding site found in the 3′ untranslated region of mRNA encoded by MGC4643, corresponding to a HOST TARGET binding site such as BINDING SITE I, BINDING SITE II or BINDING SITE III. Table 2 illustrates the complementarity of the nucleotide sequences of MGC4643 BINDING SITE, designated SEQ ID:2271, to the nucleotide sequence of VGAM71 RNA, herein designated VGAM RNA, also designated SEQ ID:406.

Another function of VGAM71 is therefore inhibition of MGC4643 (Accession NM_{—}032715). Accordingly, utilities of VGAM71 include diagnosis, prevention and treatment of diseases and clinical conditions associated with MGC4643. LOC143888 (Accession XM_{—}084669) is another VGAM71 host target gene. LOC143888 BINDING SITE is HOST TARGET binding site found in the 3′ untranslated region of mRNA encoded by LOC143888, corresponding to a HOST TARGET binding site such as BINDING SITE I, BINDING SITE II or BINDING SITE III. Table 2 illustrates the complementarity of the nucleotide sequences of LOC143888 BINDING SITE, designated SEQ ID:3064, to the nucleotide sequence of VGAM71 RNA, herein designated VGAM RNA, also designated SEQ ID:406.

Another function of VGAM71 is therefore inhibition of LOC143888 (Accession XM_{—}084669). Accordingly, utilities of VGAM71 include diagnosis, prevention and treatment of diseases and clinical conditions associated with LOC143888. LOC149619 (Accession XM_{—}097690) is another VGAM71 host target gene. LOC149619 BINDING SITE is HOST TARGET binding site found in the 5′ untranslated region of mRNA encoded by LOC149619, corresponding to a HOST TARGET binding site such as BINDING SITE I, BINDING SITE II or BINDING SITE III. Table 2 illustrates the complementarity of the nucleotide sequences of LOC149619 BINDING SITE, designated SEQ ID:3311, to the nucleotide sequence of VGAM71 RNA, herein designated VGAM RNA, also designated SEQ ID:406.

Another function of VGAM71 is therefore inhibition of LOC149619 (Accession XM_{—}097690). Accordingly, utilities of VGAM71 include diagnosis, prevention and treatment of diseases and clinical conditions associated with LOC149619. LOC91464 (Accession XM_{—}038589) is another VGAM71 host target gene. LOC91464 BINDING SITE is HOST TARGET binding site found in the 5′ untranslated region of mRNA encoded by LOC91464, corresponding to a HOST TARGET binding site such as BINDING SITE I, BINDING SITE II or BINDING SITE III. Table 2 illustrates the complementarity of the nucleotide sequences of LOC91464 BINDING SITE, designated SEQ ID:2740, to the nucleotide sequence of VGAM71 RNA, herein designated VGAM RNA, also designated SEQ ID:406.

Another function of VGAM71 is therefore inhibition of LOC91464 (Accession XM_{—}038589). Accordingly, utilities of VGAM71 include diagnosis, prevention and treatment of diseases and clinical conditions associated with LOC91464. FIG. 1 further provides a conceptual description of a novel bioinformatically detected viral gene of the present invention, referred to here as Viral Genomic Address Messenger 72 (VGAM72) viral gene, which modulates expression of respective host target genes thereof, the function and utility of which host target genes is known in the art.

VGAM72 is a novel bioinformatically detected regulatory, non protein coding, viral micro RNA (miRNA) gene. The method by which VGAM72 was detected is described hereinabove with reference to FIGS. 1-8.

VGAM72 gene, herein designated VGAM GENE, is a viral gene contained in the genome of Vaccinia Virus. VGAM72 host target gene, herein designated VGAM HOST TARGET GENE, is a human gene contained in the human genome.

VGAM72 gene encodes a VGAM72 precursor RNA, herein designated VGAM PRECURSOR RNA. Similar to other miRNA genes, and unlike most ordinary genes, VGAM72 precursor RNA does not encode a protein. A nucleotide sequence identical or highly similar to the nucleotide sequence of VGAM72 precursor RNA is designated SEQ ID:58, and is provided hereinbelow with reference to the sequence listing part. Nucleotide sequence SEQ ID:58 is located at position 14074 relative to the genome of Vaccinia Virus.

VGAM72 precursor RNA folds onto itself, forming VGAM72 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM72 folded precursor RNA into VGAM72 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM72 RNA is designated SEQ ID:407, and is provided hereinbelow with reference to the sequence listing part.

VGAM72 host target gene, herein designated VGAM HOST TARGET GENE, encodes a corresponding messenger RNA, VGAM72 host target RNA, herein designated VGAM HOST TARGET RNA. VGAM72 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM72 RNA, herein designated VGAM RNA, binds complementarily to one or more host target binding sites located in untranslated regions of VGAM72 host target RNA, herein designated VGAM HOST TARGET RNA. This complementary binding is due to the fact that the nucleotide sequence of VGAM72 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM72 RNA, herein designated VGAM RNA, may have a different number of host target binding sites in untranslated regions of a VGAM72 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM72 RNA, herein designated VGAM RNA, to host target binding sites on VGAM72 host target RNA, herein designated VGAM HOST TARGET RNA, such as BINDING SITE I, BINDING SITE II and BINDING SITE III, inhibits translation of VGAM72 host target RNA into VGAM72 host target protein, herein designated VGAM HOST TARGET PROTEIN. VGAM host target protein is therefore outlined by a broken line.

It is appreciated that VGAM72 host target gene, herein designated VGAM HOST TARGET GENE, in fact represents a plurality of VGAM72 host target genes. The mRNA of each one of this plurality of VGAM72 host target genes comprises one or more host target binding sites, each having a nucleotide sequence which is at least partly complementary to VGAM72 RNA, herein designated VGAM RNA, and which when bound by VGAM72 RNA causes inhibition of translation of respective one or more VGAM72 host target proteins.

It is further appreciated by one skilled in the art that the mode of translational inhibition illustrated by FIG. 1 with specific reference to translational inhibition exerted by VGAM72 gene, herein designated VGAM GENE, on one or more VGAM72 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM72 is inhibition of expression of host target genes, as part of a novel viral mechanism of attacking a host. Accordingly, utilities of VGAM72 include diagnosis, prevention and treatment of viral infection by Vaccinia Virus. Specific functions, and accordingly utilities, of VGAM72 correlate with, and may be deduced from, the identity of the host target genes which VGAM72 binds and inhibits, and the function of these host target genes, as elaborated hereinbelow.

Nucleotide sequences of the VGAM72 precursor RNA, herein designated VGAM PRECURSOR RNA, and of the ‘diced’ VGAM72 RNA, herein designated VGAM RNA, and a schematic representation of the secondary folding of VGAM72 folded precursor RNA, herein designated VGAM FOLDED PRECURSOR RNA, of VGAM72 are further described hereinbelow with reference to Table 1.

Nucleotide sequences of host target binding sites, such as BINDING SITE-I, BINDING SITE-II and BINDING SITE-III of FIG. 1, found on VGAM72 host target RNA, and schematic representation of the complementarity of each of these host target binding sites to VGAM72 RNA, herein designated VGAM RNA, are described hereinbelow with reference to Table 2.

As mentioned hereinabove with reference to FIG. 1, a function of VGAM72 gene, herein designated VGAM is inhibition of expression of VGAM72 target genes. It is appreciated that specific functions, and accordingly utilities, of VGAM72 correlate with, and may be deduced from, the identity of the target genes which VGAM72 binds and inhibits, and the function of these target genes, as elaborated hereinbelow.

Claudin 1 (CLDN1, Accession NM_{—}021101) is a VGAM72 host target gene. CLDN1 BINDING SITE is HOST TARGET binding site found in the 3′ untranslated region of mRNA encoded by CLDN1, corresponding to a HOST TARGET binding site such as BINDING SITE I, BINDING SITE II or BINDING SITE III. Table 2 illustrates the complementarity of the nucleotide sequences of CLDN1 BINDING SITE, designated SEQ ID:1940, to the nucleotide sequence of VGAM72 RNA, herein designated VGAM RNA, also designated SEQ ID:407.

A function of VGAM72 is therefore inhibition of Claudin 1 (CLDN1, Accession NM_{—}021101). Accordingly, utilities of VGAM72 include diagnosis, prevention and treatment of diseases and clinical conditions associated with CLDN1. KIAA1706 (Accession XM_{—}166595) is another VGAM72 host target gene. KIAA1706 BINDING SITE is HOST TARGET binding site found in the 3′ untranslated region of mRNA encoded by KIAA1706, corresponding to a HOST TARGET binding site such as BINDING SITE I, BINDING SITE II or BINDING SITE III. Table 2 illustrates the complementarity of the nucleotide sequences of KIAA1706 BINDING SITE, designated SEQ ID:3577, to the nucleotide sequence of VGAM72 RNA, herein designated VGAM RNA, also designated SEQ ID:407.

Another function of VGAM72 is therefore inhibition of KIAA1706 (Accession XM_{—}166595). Accordingly, utilities of VGAM72 include diagnosis, prevention and treatment of diseases and clinical conditions associated with KIAA1706. FIG. 1 further provides a conceptual description of a novel bioinformatically detected viral gene of the present invention, referred to here as Viral Genomic Address Messenger 73 (VGAM73) viral gene, which modulates expression of respective host target genes thereof, the function and utility of which host target genes is known in the art.

VGAM73 is a novel bioinformatically detected regulatory, non protein coding, viral micro RNA (miRNA) gene. The method by which VGAM73 was detected is described hereinabove with reference to FIGS. 1-8.

VGAM73 gene, herein designated VGAM GENE, is a viral gene contained in the genome of Vaccinia Virus. VGAM73 host target gene, herein designated VGAM HOST TARGET GENE, is a human gene contained in the human genome.

VGAM73 gene encodes a VGAM73 precursor RNA, herein designated VGAM PRECURSOR RNA. Similar to other miRNA genes, and unlike most ordinary genes, VGAM73 precursor RNA does not encode a protein. A nucleotide sequence identical or highly similar to the nucleotide sequence of VGAM73 precursor RNA is designated SEQ ID:59, and is provided hereinbelow with reference to the sequence listing part. Nucleotide sequence SEQ ID:59 is located at position 14338 relative to the genome of Vaccinia Virus.

VGAM73 precursor RNA folds onto itself, forming VGAM73 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM73 folded precursor RNA into VGAM73 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM73 RNA is designated SEQ ID:408, and is provided hereinbelow with reference to the sequence listing part.

VGAM73 host target gene, herein designated VGAM HOST TARGET GENE, encodes a corresponding messenger RNA, VGAM73 host target RNA, herein designated VGAM HOST TARGET RNA. VGAM73 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM73 RNA, herein designated VGAM RNA, binds complementarily to one or more host target binding sites located in untranslated regions of VGAM73 host target RNA, herein designated VGAM HOST TARGET RNA. This complementary binding is due to the fact that the nucleotide sequence of VGAM73 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM73 RNA, herein designated VGAM RNA, may have a different number of host target binding sites in untranslated regions of a VGAM73 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM73 RNA, herein designated VGAM RNA, to host target binding sites on VGAM73 host target RNA, herein designated VGAM HOST TARGET RNA, such as BINDING SITE I, BINDING SITE II and BINDING SITE III, inhibits translation of VGAM73 host target RNA into VGAM73 host target protein, herein designated VGAM HOST TARGET PROTEIN. VGAM host target protein is therefore outlined by a broken line.

It is appreciated that VGAM73 host target gene, herein designated VGAM HOST TARGET GENE, in fact represents a plurality of VGAM73 host target genes. The mRNA of each one of this plurality of VGAM73 host target genes comprises one or more host target binding sites, each having a nucleotide sequence which is at least partly complementary to VGAM73 RNA, herein designated VGAM RNA, and which when bound by VGAM73 RNA causes inhibition of translation of respective one or more VGAM73 host target proteins.

It is further appreciated by one skilled in the art that the mode of translational inhibition illustrated by FIG. 1 with specific reference to translational inhibition exerted by VGAM73 gene, herein designated VGAM GENE, on one or more VGAM73 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM73 is inhibition of expression of host target genes, as part of a novel viral mechanism of attacking a host. Accordingly, utilities of VGAM73 include diagnosis, prevention and treatment of viral infection by Vaccinia Virus. Specific functions, and accordingly utilities, of VGAM73 correlate with, and may be deduced from, the identity of the host target genes which VGAM73 binds and inhibits, and the function of these host target genes, as elaborated hereinbelow.

Nucleotide sequences of the VGAM73 precursor RNA, herein designated VGAM PRECURSOR RNA, and of the ‘diced’ VGAM73 RNA, herein designated VGAM RNA, and a schematic representation of the secondary folding of VGAM73 folded precursor RNA, herein designated VGAM FOLDED PRECURSOR RNA, of VGAM73 are further described hereinbelow with reference to Table 1.

Nucleotide sequences of host target binding sites, such as BINDING SITE-I, BINDING SITE-II and BINDING SITE-III of FIG. 1, found on VGAM73 host target RNA, and schematic representation of the complementarity of each of these host target binding sites to VGAM73 RNA, herein designated VGAM RNA, are described hereinbelow with reference to Table 2.

As mentioned hereinabove with reference to FIG. 1, a function of VGAM73 gene, herein designated VGAM is inhibition of expression of VGAM73 target genes. It is appreciated that specific functions, and accordingly utilities, of VGAM73 correlate with, and may be deduced from, the identity of the target genes which VGAM73 binds and inhibits, and the function of these target genes, as elaborated hereinbelow.

UDP-Gal:betaGlcNAc Beta 1,3-galactosyltransferase, Polypeptide 5 (B3GALT5, Accession NM_{—}033170) is a VGAM73 host target gene. B3GALT5 BINDING SITE1 through B3GALT5 BINDING SITES are HOST TARGET binding sites found in untranslated regions of mRNA encoded by B3GALT5, corresponding to HOST TARGET binding sites such as BINDING SITE I, BINDING SITE II or BINDING SITE III. Table 2 illustrates the complementarity of the nucleotide sequences of B3GALT5 BINDING SITE1 through B3GALT5 BINDING SITES, designated SEQ ID:2317, SEQ ID:1268, SEQ ID:2318, SEQ ID:2319 and SEQ ID:2321 respectively, to the nucleotide sequence of VGAM73 RNA, herein designated VGAM RNA, also designated SEQ ID:408.

A function of VGAM73 is therefore inhibition of UDP-Gal:betaGlcNAc Beta 1,3-galactosyltransferase, Polypeptide 5 (B3GALT5, Accession NM_{—}033170). Accordingly, utilities of VGAM73 include diagnosis, prevention and treatment of diseases and clinical conditions associated with B3GALT5. Origin Recognition Complex, Subunit 2-like (yeast) (ORC2L, Accession NM_{—}006190) is another VGAM73 host target gene. ORC2L BINDING SITE is HOST TARGET binding site found in the 3′ untranslated region of mRNA encoded by ORC2L, corresponding to a HOST TARGET binding site such as BINDING SITE I, BINDING SITE II or BINDING SITE III. Table 2 illustrates the complementarity of the nucleotide sequences of ORC2L BINDING SITE, designated SEQ ID:1278, to the nucleotide sequence of VGAM73 RNA, herein designated VGAM RNA, also designated SEQ ID:408.

Another function of VGAM73 is therefore inhibition of Origin Recognition Complex, Subunit 2-like (yeast) (ORC2L, Accession NM_{—}006190), a gene which is a subunit of the origin recognition complex and may be required for initiation of DNA replication. Accordingly, utilities of VGAM73 include diagnosis, prevention and treatment of diseases and clinical conditions associated with ORC2L. The function of ORC2L has been established by previous studies. The origin recognition complex (ORC) is a multiprotein assemblage identified in S. cerevisiae that binds to the ARS (autonomously replicating sequence) consensus, a DNA motif that is an essential part of yeast origins of replication. ORC binding has also been implicated in transcriptional silencing at certain yeast loci. One component of the ORC is a 72-kD protein designated ORC2, mutations of which disrupt silencing at the HMR-E silencer and cause cell cycle arrest between late G1 and the initiation of DNA replication. Takahara et al. (1996) isolated a cDNA from a mouse embryonic stem cell library and a human placenta library whose predicted 577-amino acid protein resembles the yeast sequence. The mouse and human sequences of the ORC2L (ORC2-like) proteins are 47.9% and 46.3% similar to yeast ORC2, respectively. Northern blots showed highest levels of ORC2L expression in testes. Dhar et al. (2001) used homologous recombination to replace the third exon encoding the initiator ATG of the ORC2 gene with a neomycin phosphotransferase gene in a colon carcinoma cell line. This hypomorphic mutation decreased ORC2 protein levels by 90%. The G1 phase of the cell cycle was prolonged, but there was no effect on the utilization of either the MYC (OMIM Ref. No. 190080) or beta-globin (OMIM Ref. No. 141900) cellular origins of replication. Cells carrying the ORC2 mutation failed to support the replication of a plasmid bearing the oriP replicator of Epstein-Barr virus (EBV), and this defect could be rescued by reintroduction of ORC2. ORC2 specifically associated with oriP in cells, most likely through its interaction with EBV nuclear antigen-1. Geminin (OMIM Ref. No. 602842), an inhibitor of the mammalian replication initiation complex, inhibited replication from oriP. Therefore, ORC and the human replication initiation apparatus is required for replication from a viral origin of replication.

Full details of the abovementioned studies are described in the following publications, the disclosure of which are hereby incorporated by reference:
Dhar, S. K.; Yoshida, K.; Machida, Y.; Khaira, P.; Chaudhuri, B.; Wohlschlegel, J. A.; Leffak, M.; Yates, J.; Dutta, A.: Replication from oriP of Epstein-Barr virus requires human ORC and is inhibited by geminin. Cell 106: 287-296, 2001.; and
Takahara, K.; Bong, M.; Brevard, R.; Eddy, R. L.; Haley, L. L.; Sait, S. J.; Shows, T. B.; Hoffman, G. G.; Greenspan, D. S.: Mouse and human homologues of the yeast origin of replica.

Further studies establishing the function and utilities of ORC2L are found in John Hopkins OMIM database record ID 601182, and in sited publications numbered 2142-2145 listed in the bibliography section hereinbelow, which are also hereby incorporated by reference. PART1 (Accession NM_{—}016590) is another VGAM73 host target gene. PART1 BINDING SITE is HOST TARGET binding site found in the 3′ untranslated region of mRNA encoded by PART1, corresponding to a HOST TARGET binding site such as BINDING SITE I, BINDING SITE II or BINDING SITE III. Table 2 illustrates the complementarity of the nucleotide sequences of PART1 BINDING SITE, designated SEQ ID: 1699, to the nucleotide sequence of VGAM73 RNA, herein designated VGAM RNA, also designated SEQ ID:408.

Another function of VGAM73 is therefore inhibition of PART1 (Accession NM_{—}016590). Accordingly, utilities of VGAM73 include diagnosis, prevention and treatment of diseases and clinical conditions associated with PART1. Solute Carrier Family 10 (sodium/bile acid cotransporter family), Member 1 (SLC10A1, Accession NM_{—}003049) is another VGAM73 host target gene. SLC10A1 BINDING SITE is HOST TARGET binding site found in the 3′ untranslated region of mRNA encoded by SLC10A1, corresponding to a HOST TARGET binding site such as BINDING SITE I, BINDING SITE II or BINDING SITE III. Table 2 illustrates the complementarity of the nucleotide sequences of SLC10A1 BINDING SITE, designated SEQ ID:989, to the nucleotide sequence of VGAM73 RNA, herein designated VGAM RNA, also designated SEQ ID:408.

Another function of VGAM73 is therefore inhibition of Solute Carrier Family 10 (sodium/bile acid cotransporter family), Member 1 (SLC10A1, Accession NM_{—}003049). Accordingly, utilities of VGAM73 include diagnosis, prevention and treatment of diseases and clinical conditions associated with SLC10A1. FUS Interacting Protein (serine-arginine rich) 1 (FUSIP1, Accession NM_{—}006625) is another VGAM73 host target gene. FUSIP1 BINDING SITE is HOST TARGET binding site found in the 3′ untranslated region of mRNA encoded by FUSIP1, corresponding to a HOST TARGET binding site such as BINDING SITE I, BINDING SITE II or BINDING SITE III. Table 2 illustrates the complementarity of the nucleotide sequences of FUSIP1 BINDING SITE, designated SEQ ID:1314, to the nucleotide sequence of VGAM73 RNA, herein designated VGAM RNA, also designated SEQ ID:408.

Another function of VGAM73 is therefore inhibition of FUS Interacting Protein (serine-arginine rich) 1 (FUSIP1, Accession NM_{—}006625). Accordingly, utilities of VGAM73 include diagnosis, prevention and treatment of diseases and clinical conditions associated with FUSIP1. Retinoic Acid Induced 17 (RAI17, Accession XM_{—}166091) is another VGAM73 host target gene. RAI17 BINDING SITE is HOST TARGET binding site found in the 3′ untranslated region of mRNA encoded by RAI17, corresponding to a HOST TARGET binding site such as BINDING SITE I, BINDING SITE II or BINDING SITE III. Table 2 illustrates the complementarity of the nucleotide sequences of RAI17 BINDING SITE, designated SEQ ID:3515, to the nucleotide sequence of VGAM73 RNA, herein designated VGAM RNA, also designated SEQ ID:408.

Another function of VGAM73 is therefore inhibition of Retinoic Acid Induced 17 (RAI17, Accession XM_{—}166091). Accordingly, utilities of VGAM73 include diagnosis, prevention and treatment of diseases and clinical conditions associated with RAI17. LOC115811 (Accession NM_{—}138451) is another VGAM73 host target gene. LOC115811 BINDING SITE is HOST TARGET binding site found in the 5′ untranslated region of mRNA encoded by LOC115811, corresponding to a HOST TARGET binding site such as BINDING SITE I, BINDING SITE II or BINDING SITE III. Table 2 illustrates the complementarity of the nucleotide sequences of LOC115811 BINDING SITE, designated SEQ ID:2442, to the nucleotide sequence of VGAM73 RNA, herein designated VGAM RNA, also designated SEQ ID:408.

Another function of VGAM73 is therefore inhibition of LOC115811 (Accession NM_{—}138451). Accordingly, utilities of VGAM73 include diagnosis, prevention and treatment of diseases and clinical conditions associated with LOC115811. LOC256946 (Accession XM_{—}170543) is another VGAM73 host target gene. LOC256946 BINDING SITE is HOST TARGET binding site found in the 3′ untranslated region of mRNA encoded by LOC256946, corresponding to a HOST TARGET binding site such as BINDING SITE I, BINDING SITE II or BINDING SITE III. Table 2 illustrates the complementarity of the nucleotide sequences of LOC256946 BINDING SITE, designated SEQ ID:3657, to the nucleotide sequence of VGAM73 RNA, herein designated VGAM RNA, also designated SEQ ID:408.

Another function of VGAM73 is therefore inhibition of LOC256946 (Accession XM_{—}170543). Accordingly, utilities of VGAM73 include diagnosis, prevention and treatment of diseases and clinical conditions associated with LOC256946. FIG. 1 further provides a conceptual description of a novel bioinformatically detected viral gene of the present invention, referred to here as Viral Genomic Address Messenger 74 (VGAM74) viral gene, which modulates expression of respective host target genes thereof, the function and utility of which host target genes is known in the art.

VGAM74 is a novel bioinformatically detected regulatory, non protein coding, viral micro RNA (miRNA) gene. The method by which VGAM74 was detected is described hereinabove with reference to FIGS. 1-8.

VGAM74 gene, herein designated VGAM GENE, is a viral gene contained in the genome of Vaccinia Virus. VGAM74 host target gene, herein designated VGAM HOST TARGET GENE, is a human gene contained in the human genome.

VGAM74 gene encodes a VGAM74 precursor RNA, herein designated VGAM PRECURSOR RNA. Similar to other miRNA genes, and unlike most ordinary genes, VGAM74 precursor RNA does not encode a protein. A nucleotide sequence identical or highly similar to the nucleotide sequence of VGAM74 precursor RNA is designated SEQ ID:60, and is provided hereinbelow with reference to the sequence listing part. Nucleotide sequence SEQ ID:60 is located at position 15206 relative to the genome of Vaccinia Virus.

VGAM74 precursor RNA folds onto itself, forming VGAM74 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM74 folded precursor RNA into VGAM74 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM74 RNA is designated SEQ ID:409, and is provided hereinbelow with reference to the sequence listing part.

VGAM74 host target gene, herein designated VGAM HOST TARGET GENE, encodes a corresponding messenger RNA, VGAM74 host target RNA, herein designated VGAM HOST TARGET RNA. VGAM74 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM74 RNA, herein designated VGAM RNA, binds complementarily to one or more host target binding sites located in untranslated regions of VGAM74 host target RNA, herein designated VGAM HOST TARGET RNA. This complementary binding is due to the fact that the nucleotide sequence of VGAM74 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM74 RNA, herein designated VGAM RNA, may have a different number of host target binding sites in untranslated regions of a VGAM74 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM74 RNA, herein designated VGAM RNA, to host target binding sites on VGAM74 host target RNA, herein designated VGAM HOST TARGET RNA, such as BINDING SITE I, BINDING SITE II and BINDING SITE III, inhibits translation of VGAM74 host target RNA into VGAM74 host target protein, herein designated VGAM HOST TARGET PROTEIN. VGAM host target protein is therefore outlined by a broken line.

It is appreciated that VGAM74 host target gene, herein designated VGAM HOST TARGET GENE, in fact represents a plurality of VGAM74 host target genes. The mRNA of each one of this plurality of VGAM74 host target genes comprises one or more host target binding sites, each having a nucleotide sequence which is at least partly complementary to VGAM74 RNA, herein designated VGAM RNA, and which when bound by VGAM74 RNA causes inhibition of translation of respective one or more VGAM74 host target proteins.

It is further appreciated by one skilled in the art that the mode of translational inhibition illustrated by FIG. 1 with specific reference to translational inhibition exerted by VGAM74 gene, herein designated VGAM GENE, on one or more VGAM74 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM74 is inhibition of expression of host target genes, as part of a novel viral mechanism of attacking a host. Accordingly, utilities of VGAM74 include diagnosis, prevention and treatment of viral infection by Vaccinia Virus. Specific functions, and accordingly utilities, of VGAM74 correlate with, and may be deduced from, the identity of the host target genes which VGAM74 binds and inhibits, and the function of these host target genes, as elaborated hereinbelow.

Nucleotide sequences of the VGAM74 precursor RNA, herein designated VGAM PRECURSOR RNA, and of the ‘diced’ VGAM74 RNA, herein designated VGAM RNA, and a schematic representation of the secondary folding of VGAM74 folded precursor RNA, herein designated VGAM FOLDED PRECURSOR RNA, of VGAM74 are further described hereinbelow with reference to Table 1.

Nucleotide sequences of host target binding sites, such as BINDING SITE-I, BINDING SITE-II and BINDING SITE-III of FIG. 1, found on VGAM74 host target RNA, and schematic representation of the complementarity of each of these host target binding sites to VGAM74 RNA, herein designated VGAM RNA, are described hereinbelow with reference to Table 2.

As mentioned hereinabove with reference to FIG. 1, a function of VGAM74 gene, herein designated VGAM is inhibition of expression of VGAM74 target genes. It is appreciated that specific functions, and accordingly utilities, of VGAM74 correlate with, and may be deduced from, the identity of the target genes which VGAM74 binds and inhibits, and the function of these target genes, as elaborated hereinbelow.

Peroxisomal Biogenesis Factor 3 (PEX3, Accession NM_{—}003630) is a VGAM74 host target gene. PEX3 BINDING SITE is HOST TARGET binding site found in the 3′ untranslated region of mRNA encoded by PEX3, corresponding to a HOST TARGET binding site such as BINDING SITE I, BINDING SITE II or BINDING SITE III. Table 2 illustrates the complementarity of the nucleotide sequences of PEX3 BINDING SITE, designated SEQ ID: 1044, to the nucleotide sequence of VGAM74 RNA, herein designated VGAM RNA, also designated SEQ ID:409.

A function of VGAM74 is therefore inhibition of Peroxisomal Biogenesis Factor 3 (PEX3, Accession NM_{—}003630). Accordingly, utilities of VGAM74 include diagnosis, prevention and treatment of diseases and clinical conditions associated with PEX3. Polycystic Kidney Disease 2-like 2 (PKD2L2, Accession NM_{—}014386) is another VGAM74 host target gene. PKD2L2 BINDING SITE is HOST TARGET binding site found in the 3′ untranslated region of mRNA encoded by PKD2L2, corresponding to a HOST TARGET binding site such as BINDING SITE I, BINDING SITE II or BINDING SITE III. Table 2 illustrates the complementarity of the nucleotide sequences of PKD2L2 BINDING SITE, designated SEQ ID: 1497, to the nucleotide sequence of VGAM74 RNA, herein designated VGAM RNA, also designated SEQ ID:409.

Another function of VGAM74 is therefore inhibition of Polycystic Kidney Disease 2-like 2 (PKD2L2, Accession NM_{—}014386). Accordingly, utilities of VGAM74 include diagnosis, prevention and treatment of diseases and clinical conditions associated with PKD2L2. BA108L7.2 (Accession NM_{—}030971) is another VGAM74 host target gene. BA108L7.2 BINDING SITE is HOST TARGET binding site found in the 3′ untranslated region of mRNA encoded by BA108L7.2, corresponding to a HOST TARGET binding site such as BINDING SITE I, BINDING SITE II or BINDING SITE III. Table 2 illustrates the complementarity of the nucleotide sequences of BA108L7.2 BINDING SITE, designated SEQ ID:2179, to the nucleotide sequence of VGAM74 RNA, herein designated VGAM RNA, also designated SEQ ID:409.

Another function of VGAM74 is therefore inhibition of BA108L7.2 (Accession NM_{—}030971). Accordingly, utilities of VGAM74 include diagnosis, prevention and treatment of diseases and clinical conditions associated with BA108L7.2. Isopentenyl-diphosphate Delta Isomerase (IDI1, Accession XM_{—}040272) is another VGAM74 host target gene. IDI1 BINDING SITE is HOST TARGET binding site found in the 3′ untranslated region of mRNA encoded by IDI1, corresponding to a HOST TARGET binding site such as BINDING SITE I, BINDING SITE II or BINDING SITE III. Table 2 illustrates the complementarity of the nucleotide sequences of IDI1 BINDING SITE, designated SEQ ID:2776, to the nucleotide sequence of VGAM74 RNA, herein designated VGAM RNA, also designated SEQ ID:409.

Another function of VGAM74 is therefore inhibition of Isopentenyl-diphosphate Delta Isomerase (IDI1, Accession XM_{—}040272). Accordingly, utilities of VGAM74 include diagnosis, prevention and treatment of diseases and clinical conditions associated with IDI1. PRO0899 (Accession NM_{—}018565) is another VGAM74 host target gene. PRO0899 BINDING SITE is HOST TARGET binding site found in the 5′ untranslated region of mRNA encoded by PRO0899, corresponding to a HOST TARGET binding site such as BINDING SITE I, BINDING SITE II or BINDING SITE III. Table 2 illustrates the complementarity of the nucleotide sequences of PRO0899 BINDING SITE, designated SEQ ID:1846, to the nucleotide sequence of VGAM74 RNA, herein designated VGAM RNA, also designated SEQ ID:409.

Another function of VGAM74 is therefore inhibition of PRO0899 (Accession NM_{—}018565). Accordingly, utilities of VGAM74 include diagnosis, prevention and treatment of diseases and clinical conditions associated with PRO0899. SEC14-like 1 (S. cerevisiae) (SEC14Li, Accession NM_{—}003003) is another VGAM74 host target gene. SEC14L1 BINDING SITE is HOST TARGET binding site found in the 3′ untranslated region of mRNA encoded by SEC14Li, corresponding to a HOST TARGET binding site such as BINDING SITE I, BINDING SITE II or BINDING SITE III. Table 2 illustrates the complementarity of the nucleotide sequences of SEC14L1 BINDING SITE, designated SEQ ID:980, to the nucleotide sequence of VGAM74 RNA, herein designated VGAM RNA, also designated SEQ ID:409.

Another function of VGAM74 is therefore inhibition of SEC14-like 1 (S. cerevisiae) (SEC14Li, Accession NM_{—}003003). Accordingly, utilities of VGAM74 include diagnosis, prevention and treatment of diseases and clinical conditions associated with SEC14L1. LOC130074 (Accession XM_{—}072228) is another VGAM74 host target gene. LOC130074 BINDING SITE is HOST TARGET binding site found in the 3′ untranslated region of mRNA encoded by LOC130074, corresponding to a HOST TARGET binding site such as BINDING SITE I, BINDING SITE II or BINDING SITE III. Table 2 illustrates the complementarity of the nucleotide sequences of LOC130074 BINDING SITE, designated SEQ ID:3045, to the nucleotide sequence of VGAM74 RNA, herein designated VGAM RNA, also designated SEQ ID:409.

Another function of VGAM74 is therefore inhibition of LOC130074 (Accession XM_{—}072228). Accordingly, utilities of VGAM74 include diagnosis, prevention and treatment of diseases and clinical conditions associated with LOC130074. LOC222159 (Accession XM_{—}168421) is another VGAM74 host target gene. LOC222159 BINDING SITE is HOST TARGET binding site found in the 5′ untranslated region of mRNA encoded by LOC222159, corresponding to a HOST TARGET binding site such as BINDING SITE I, BINDING SITE II or BINDING SITE III. Table 2 illustrates the complementarity of the nucleotide sequences of LOC222159 BINDING SITE, designated SEQ ID:3633, to the nucleotide sequence of VGAM74 RNA, herein designated VGAM RNA, also designated SEQ ID:409.

Another function of VGAM74 is therefore inhibition of LOC222159 (Accession XM_{—}168421). Accordingly, utilities of VGAM74 include diagnosis, prevention and treatment of diseases and clinical conditions associated with LOC222159. FIG. 1 further provides a conceptual description of a novel bioinformatically detected viral gene of the present invention, referred to here as Viral Genomic Address Messenger 75 (VGAM75) viral gene, which modulates expression of respective host target genes thereof, the function and utility of which host target genes is known in the art.

VGAM75 is a novel bioinformatically detected regulatory, non protein coding, viral micro RNA (miRNA) gene. The method by which VGAM75 was detected is described hereinabove with reference to FIGS. 1-8.

VGAM75 gene, herein designated VGAM GENE, is a viral gene contained in the genome of Vaccinia Virus. VGAM75 host target gene, herein designated VGAM HOST TARGET GENE, is a human gene contained in the human genome.

VGAM75 gene encodes a VGAM75 precursor RNA, herein designated VGAM PRECURSOR RNA. Similar to other miRNA genes, and unlike most ordinary genes, VGAM75 precursor RNA does not encode a protein. A nucleotide sequence identical or highly similar to the nucleotide sequence of VGAM75 precursor RNA is designated SEQ ID:61, and is provided hereinbelow with reference to the sequence listing part. Nucleotide sequence SEQ ID:61 is located at position 14680 relative to the genome of Vaccinia Virus.

VGAM75 precursor RNA folds onto itself, forming VGAM75 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM75 folded precursor RNA into VGAM75 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM75 RNA is designated SEQ ID:410, and is provided hereinbelow with reference to the sequence listing part.

VGAM75 host target gene, herein designated VGAM HOST TARGET GENE, encodes a corresponding messenger RNA, VGAM75 host target RNA, herein designated VGAM HOST TARGET RNA. VGAM75 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM75 RNA, herein designated VGAM RNA, binds complementarily to one or more host target binding sites located in untranslated regions of VGAM75 host target RNA, herein designated VGAM HOST TARGET RNA. This complementary binding is due to the fact that the nucleotide sequence of VGAM75 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM75 RNA, herein designated VGAM RNA, may have a different number of host target binding sites in untranslated regions of a VGAM75 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM75 RNA, herein designated VGAM RNA, to host target binding sites on VGAM75 host target RNA, herein designated VGAM HOST TARGET RNA, such as BINDING SITE I, BINDING SITE II and BINDING SITE III, inhibits translation of VGAM75 host target RNA into VGAM75 host target protein, herein designated VGAM HOST TARGET PROTEIN. VGAM host target protein is therefore outlined by a broken line.

It is appreciated that VGAM75 host target gene, herein designated VGAM HOST TARGET GENE, in fact represents a plurality of VGAM75 host target genes. The mRNA of each one of this plurality of VGAM75 host target genes comprises one or more host target binding sites, each having a nucleotide sequence which is at least partly complementary to VGAM75 RNA, herein designated VGAM RNA, and which when bound by VGAM75 RNA causes inhibition of translation of respective one or more VGAM75 host target proteins.

It is further appreciated by one skilled in the art that the mode of translational inhibition illustrated by FIG. 1 with specific reference to translational inhibition exerted by VGAM75 gene, herein designated VGAM GENE, on one or more VGAM75 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM75 is inhibition of expression of host target genes, as part of a novel viral mechanism of attacking a host. Accordingly, utilities of VGAM75 include diagnosis, prevention and treatment of viral infection by Vaccinia Virus. Specific functions, and accordingly utilities, of VGAM75 correlate with, and may be deduced from, the identity of the host target genes which VGAM75 binds and inhibits, and the function of these host target genes, as elaborated hereinbelow.

Nucleotide sequences of the VGAM75 precursor RNA, herein designated VGAM PRECURSOR RNA, and of the ‘diced’ VGAM75 RNA, herein designated VGAM RNA, and a schematic representation of the secondary folding of VGAM75 folded precursor RNA, herein designated VGAM FOLDED PRECURSOR RNA, of VGAM75 are further described hereinbelow with reference to Table 1.

Nucleotide sequences of host target binding sites, such as BINDING SITE-I, BINDING SITE-II and BINDING SITE-III of FIG. 1, found on VGAM75 host target RNA, and schematic representation of the complementarity of each of these host target binding sites to VGAM75 RNA, herein designated VGAM RNA, are described hereinbelow with reference to Table 2.

As mentioned hereinabove with reference to FIG. 1, a function of VGAM75 gene, herein designated VGAM is inhibition of expression of VGAM75 target genes. It is appreciated that specific functions, and accordingly utilities, of VGAM75 correlate with, and may be deduced from, the identity of the target genes which VGAM75 binds and inhibits, and the function of these target genes, as elaborated hereinbelow.

Ring Finger Protein 28 (RNF28, Accession NM_{—}032588) is a VGAM75 host target gene. RNF28 BINDING SITE is HOST TARGET binding site found in the 3′ untranslated region of mRNA encoded by RNF28, corresponding to a HOST TARGET binding site such as BINDING SITE I, BINDING SITE II or BINDING SITE III. Table 2 illustrates the complementarity of the nucleotide sequences of RNF28 BINDING SITE, designated SEQ ID:2263, to the nucleotide sequence of VGAM75 RNA, herein designated VGAM RNA, also designated SEQ ID:410.

A function of VGAM75 is therefore inhibition of Ring Finger Protein 28 (RNF28, Accession NM_{—}032588), a gene which is a ubiquitin ligase and that it is expressed selectively in cardiac and skeletal muscle. Accordingly, utilities of VGAM75 include diagnosis, prevention and treatment of diseases and clinical conditions associated with RNF28. The function of RNF28 has been established by previous studies. To identify candidate molecular mediators of muscle atrophy, Bodine et al. (2001) performed transcript profiling. Although many genes were up-regulated in a single rat model of atrophy, only a small subset was universal in all atrophy models (denervation, immobilization, and unweighting). Two of these genes encode ubiquitin ligases: MURF1, and a gene designated ‘muscle atrophy F-box’ (MAFBX; 606604). Bodine et al. (2001) generated mice deficient in Murf1 by targeted disruption. Murf1 −/− mice were viable and fertile and appeared normal. They had normal growth curves relative to those of wildtype littermates, and skeletal muscles and heart muscle had normal weights and morphology. After denervation, Murf1 −/− mice had significant muscle sparing relative to wildtype littermates at 14 days but not at 7 days. Bodine et al. (2001) demonstrated that MURF1 is a ubiquitin ligase and that it is expressed selectively in cardiac and skeletal muscle Centner et al. (2001) found that RNF28 binds in vitro to the titin repeats A168/A169 adjacent to the titin kinase domain. In myofibrils, RNF28 is present within the periphery of the M-line lattice in close proximity to the catalytic kinase domain of titin, within the Z-line lattice, and also in soluble form within the cytoplasm

Full details of the abovementioned studies are described in the following publications, the disclosure of which are hereby incorporated by reference:
Bodine, S. C.; Latres, E.; Baumhueter, S.; Lai, V. K.-M.; Nunez, L.; Clarke, B. A.; Poueymirou, W. T.; Panaro, F. J.; Na, E.; Dharmarajan, K.; Pan, Z.-Q.; Valenzuela, D. M.; DeChiara, T. M.; Stitt, T. N.; Yancopoulos, G. D.; Glass, D. J.: Identification of ubiquitin ligases required for skeletal muscle atrophy. Science 294: 1704-1708, 2001.; and
Centner, T.; Yano, J.; Kimura, E.; McElhinny, A. S.; Pelin, K.; Witt, C. C.; Bang, M.-L.; Trombitas, K.; Granzier, H.; Gregorio, C. C.; Sorimachi, H.; Labeit, S.: Identification of musc.

Further studies establishing the function and utilities of RNF28 are found in John Hopkins OMIM database record ID 606131, and in sited publications numbered 1501-150 and 1961 listed in the bibliography section hereinbelow, which are also hereby incorporated by reference. SWAP70 (Accession XM_{—}049197) is another VGAM75 host target gene. SWAP70 BINDING SITE is HOST TARGET binding site found in the 3′ untranslated region of mRNA encoded by SWAP70, corresponding to a HOST TARGET binding site such as BINDING SITE I, BINDING SITE II or BINDING SITE III. Table 2 illustrates the complementarity of the nucleotide sequences of SWAP70 BINDING SITE, designated SEQ ID:2916, to the nucleotide sequence of VGAM75 RNA, herein designated VGAM RNA, also designated SEQ ID:410.

Another function of VGAM75 is therefore inhibition of SWAP70 (Accession XM_{—}049197), a gene which is involved not only in nuclear events but also in signaling in B-cell activation. Accordingly, utilities of VGAM75 include diagnosis, prevention and treatment of diseases and clinical conditions associated with SWAP70. The function of SWAP70 has been established by previous studies. The B-cell receptor is composed of the immunoglobulin (Ig) heavy and light chains and the covalently bound accessory molecules Ig-alpha (CD79A; 112205) and Ig-beta (CD79B; 147245). Crosslinking of the B-cell receptor by antigens stimulates the activation of intracellular protein kinases. B-cell activation leads to hypermutation of the Ig variable regions and to heavy chain class switching, in which the Ig constant region of mu (IgM; OMIM Ref. No. 147020) is replaced by that of another class: gamma (IgG; OMIM Ref. No. 147100), alpha (IgA; OMIM Ref. No. 146900), or epsilon (IgE; OMIM Ref. No. 147180). Class switching is achieved by a looping out and deletion mechanism between the switch region of mu and the switch region of the isotope that is to be expressed. Masat et al. (2000) explored the possibility that switch-associated protein-70 (OMIM Ref. No. SWAP70) acts as a link between the recognition of specific switch regions and causation of a DNA break. Swap70 had been isolated in the mouse as part of a complex that is able to promote recombination between 2 switch regions in vitro (Borggrefe et al., 1998; Borggrefe et al., 1999). By screening a human lymphoma cDNA library using mouse Swap70 sequences as the probe, Masat et al. (2000) isolated a cDNA encoding SWAP70. Although the 585-amino acid SWAP70 protein contains 3 nuclear localization signals, SWAP70 was found mainly in the cytoplasm in small resting B cells. On stimulation, SWAP70 translocated to the nucleus. In activated, class-switching B cell cultures, it was associated with membrane IgG, but not IgM. Masat et al. (2000) suggested that SWAP70 is involved not only in nuclear events but also in signaling in B-cell activation. Shinohara et al. (2002) demonstrated that SWAP70 specifically binds phosphatidylinositol-3,4,5-triphosphate. On stimulation by growth factors, cytoplasmic SWAP70, which is dependent on phosphoinositide-3-hydroxykinase but independent of Ras (see OMIM Ref. No. 190020), moved to cell membrane rearrangements known as ruffles. However, mutant SWAP70 lacking the ability to bind phosphatidylinositol-3,4,5-triphosphate blocked membrane ruffling induced by epidermal growth factor (EGF; 131530) or platelet-derived growth factor (see OMIM Ref. No. 173430). SWAP70 shows low homology with Rac-guanine nucleotide exchange factors, and catalyzes phosphatidylinositol-3,4,5-triphosphate-dependent guanine nucleotide exchange to Rac (see OMIM Ref. No. 602048). SWAP70-deficient fibroblasts showed impaired membrane ruffling after stimulation with EGF, and failed to activate Rac fully. Shinohara et al. (2002) concluded that SWAP70 is a different type of Rac-GEF which, independently of Ras, transduces signals from tyrosine kinase receptors to Rac.

Full details of the abovementioned studies are described in the following publications, the disclosure of which are hereby incorporated by reference:
Masat, L.; Caldwell, J.; Armstrong, R.; Khoshnevisan, H.; Jessberger, R.; Herndier, B.; Wabl, M.; Ferrick, D.: Association of SWAP-70 with the B cell antigen receptor complex. Proc. Nat. Acad. Sci. 97: 2180-2184, 2000.; and
Shinohara, M.; Terada, Y.; Iwamatsu, A.; Shinohara, A.; Mochizuki, N.; Higuchi, M.; Gotoh, Y.; Ihara, S.; Nagata, S.; Itoh, H.; Fukui, Y.; Jessberger, R.: SWAP-70 is a guanine-nucleotide.

Further studies establishing the function and utilities of SWAP70 are found in John Hopkins OMIM database record ID 604762, and in sited publications numbered 1645-1649 listed in the bibliography section hereinbelow, which are also hereby incorporated by reference. bA430M15.1 (Accession XM_{—}084997) is another VGAM75 host target gene. bA430M15.1 BINDING SITE is HOST TARGET binding site found in the 3′ untranslated region of mRNA encoded by bA430M15.1, corresponding to a HOST TARGET binding site such as BINDING SITE I, BINDING SITE II or BINDING SITE III. Table 2 illustrates the complementarity of the nucleotide sequences of bA430M15.1 BINDING SITE, designated SEQ ID:3072, to the nucleotide sequence of VGAM75 RNA, herein designated VGAM RNA, also designated SEQ ID:410.

Another function of VGAM75 is therefore inhibition of bA430M15.1 (Accession XM_{—}084997). Accordingly, utilities of VGAM75 include diagnosis, prevention and treatment of diseases and clinical conditions associated with bA430M15.1. FIG. 1 further provides a conceptual description of a novel bioinformatically detected viral gene of the present invention, referred to here as Viral Genomic Address Messenger 76 (VGAM76) viral gene, which modulates expression of respective host target genes thereof, the function and utility of which host target genes is known in the art.

VGAM76 is a novel bioinformatically detected regulatory, non protein coding, viral micro RNA (miRNA) gene. The method by which VGAM76 was detected is described hereinabove with reference to FIGS. 1-8.

VGAM76 gene, herein designated VGAM GENE, is a viral gene contained in the genome of Vaccinia Virus. VGAM76 host target gene, herein designated VGAM HOST TARGET GENE, is a human gene contained in the human genome.

VGAM76 gene encodes a VGAM76 precursor RNA, herein designated VGAM PRECURSOR RNA. Similar to other miRNA genes, and unlike most ordinary genes, VGAM76 precursor RNA does not encode a protein. A nucleotide sequence identical or highly similar to the nucleotide sequence of VGAM76 precursor RNA is designated SEQ ID:62, and is provided hereinbelow with reference to the sequence listing part. Nucleotide sequence SEQ ID:62 is located at position 15884 relative to the genome of Vaccinia Virus.

VGAM76 precursor RNA folds onto itself, forming VGAM76 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM76 folded precursor RNA into VGAM76 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM76 RNA is designated SEQ ID:411, and is provided hereinbelow with reference to the sequence listing part.

VGAM76 host target gene, herein designated VGAM HOST TARGET GENE, encodes a corresponding messenger RNA, VGAM76 host target RNA, herein designated VGAM HOST TARGET RNA. VGAM76 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM76 RNA, herein designated VGAM RNA, binds complementarily to one or more host target binding sites located in untranslated regions of VGAM76 host target RNA, herein designated VGAM HOST TARGET RNA. This complementary binding is due to the fact that the nucleotide sequence of VGAM76 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM76 RNA, herein designated VGAM RNA, may have a different number of host target binding sites in untranslated regions of a VGAM76 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM76 RNA, herein designated VGAM RNA, to host target binding sites on VGAM76 host target RNA, herein designated VGAM HOST TARGET RNA, such as BINDING SITE I, BINDING SITE II and BINDING SITE III, inhibits translation of VGAM76 host target RNA into VGAM76 host target protein, herein designated VGAM HOST TARGET PROTEIN. VGAM host target protein is therefore outlined by a broken line.

It is appreciated that VGAM76 host target gene, herein designated VGAM HOST TARGET GENE, in fact represents a plurality of VGAM76 host target genes. The mRNA of each one of this plurality of VGAM76 host target genes comprises one or more host target binding sites, each having a nucleotide sequence which is at least partly complementary to VGAM76 RNA, herein designated VGAM RNA, and which when bound by VGAM76 RNA causes inhibition of translation of respective one or more VGAM76 host target proteins.

It is further appreciated by one skilled in the art that the mode of translational inhibition illustrated by FIG. 1 with specific reference to translational inhibition exerted by VGAM76 gene, herein designated VGAM GENE, on one or more VGAM76 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM76 is inhibition of expression of host target genes, as part of a novel viral mechanism of attacking a host. Accordingly, utilities of VGAM76 include diagnosis, prevention and treatment of viral infection by Vaccinia Virus. Specific functions, and accordingly utilities, of VGAM76 correlate with, and may be deduced from, the identity of the host target genes which VGAM76 binds and inhibits, and the function of these host target genes, as elaborated hereinbelow.

Nucleotide sequences of the VGAM76 precursor RNA, herein designated VGAM PRECURSOR RNA, and of the ‘diced’ VGAM76 RNA, herein designated VGAM RNA, and a schematic representation of the secondary folding of VGAM76 folded precursor RNA, herein designated VGAM FOLDED PRECURSOR RNA, of VGAM76 are further described hereinbelow with reference to Table 1.

Nucleotide sequences of host target binding sites, such as BINDING SITE-I, BINDING SITE-II and BINDING SITE-III of FIG. 1, found on VGAM76 host target RNA, and schematic representation of the complementarity of each of these host target binding sites to VGAM76 RNA, herein designated VGAM RNA, are described hereinbelow with reference to Table 2.

As mentioned hereinabove with reference to FIG. 1, a function of VGAM76 gene, herein designated VGAM is inhibition of expression of VGAM76 target genes. It is appreciated that specific functions, and accordingly utilities, of VGAM76 correlate with, and may be deduced from, the identity of the target genes which VGAM76 binds and inhibits, and the function of these target genes, as elaborated hereinbelow.

Pregnancy Specific Beta-1-glycoprotein 1 (PSG1, Accession NM_{—}006905) is a VGAM76 host target gene. PSG1 BINDING SITE is HOST TARGET binding site found in the 3′ untranslated region of mRNA encoded by PSG1, corresponding to a HOST TARGET binding site such as BINDING SITE I, BINDING SITE II or BINDING SITE III. Table 2 illustrates the complementarity of the nucleotide sequences of PSG1 BINDING SITE, designated SEQ ID:1337, to the nucleotide sequence of VGAM76 RNA, herein designated VGAM RNA, also designated SEQ ID:411.

A function of VGAM76 is therefore inhibition of Pregnancy Specific Beta-1-glycoprotein 1 (PSG1, Accession NM_{—}006905), a gene which is a member of the pregnancy-specific glycoprotein (PSG) and CEA families. Accordingly, utilities of VGAM76 include diagnosis, prevention and treatment of diseases and clinical conditions associated with PSG1. The function of PSG1 has been established by previous studies. The human placenta is a multi-hormonal endocrine organ which produces hormones, enzymes, and other molecules that support fetal survival and development. Chorionic gonadotropin (118850, 118860), placental lactogen (OMIM Ref. No. 150200), and placental alkaline phosphatase (OMIM Ref. No. 171800) are 3 of the best-known placental proteins. Immunochemical studies on the protein composition of the serum in human pregnancy revealed several so-called pregnancy-specific proteins, including pregnancy-specific beta-1-glycoprotein (PSBG). PSBG becomes detectable in serum during the first 2 to 3 weeks of pregnancy and increases as pregnancy progresses, rising to a very high level of 200 to 400 micrograms per milliliter. Although its exact physiologic role during pregnancy is unknown, the high quantity produced by the placenta argues for its importance. PSBG isolated from human placenta consists of a set of at least 3 glycoproteins with apparent molecular masses of 72, 64, and 54 kD, respectively. The heterogeneity is confirmed by detection of 3 nonglycosylated polypeptides of 50, 48, and 36 kD, which can be immunoprecipitated by antiserum to placental PSBG obtained by in vitro translation of placental polyadenylated RNA. Watanabe and Chou (1988) examined the structural relationship of these proteins by the isolation and study of cDNA clones The members of the CEA/PSG gene family have a characteristic N-terminal domain that is homologous to the immunoglobulin variable region. Khan et al. (1992) estimated the size of the PSG sub family by identification of N-domain exons from isolated genomic clones and from total genomic DNA through PCR amplification and DNA sequence determination. They found that the PSG sub family contains at least 11 different genes. For 7 of these, 2 DNA sequences differing from each other in 1 to 4 nucleotides were detected. Most likely they represent different alleles. All of the PSGs except PSG1, PSG4 (OMIM Ref. No. 176393), and PSG8 (OMIM Ref. No. 176397) contain the arginine-glycine-aspartic acid sequence at position 93-95 corresponding to the complementarity determining region 3 of immunoglobulin. Parsimony analysis of 24 CEA and PSG sequences using 12 members of the immunoglobulin gene super family as out-groups to root the family tree showed that the N-domain of the CEA group genes evolved in one major branch and the PSG group genes in the other. Brandriff et al. (1992) estimated that the CEA-PSG gene family region spans 1.1 to 1.2 Mb. Using a high-resolution restriction fragment fingerprinting technique, Olsen et al. (1994) assembled 256 cosmids spanning the PSG region on 19q13.2 into a single 700-kb contig. FISH to sperm pronuclei and cosmid walking experiments indicated that this PSG contig is telomeric of CGM8 at the telomeric end of the CEA subgroup gene cluster. Detailed restriction mapping and hybridization with gene-specific probes indicated that the order of the 11 PSG genes in the contig is cen—PSG3 (OMIM Ref. No. 176392)—PSG8—PSG12 (PSG10; 176399)—PSG1—PSG6 (OMIM Ref. No. 176395)—PSG7 (OMIM Ref. No. 176396)—PSG13 (PSG11; 176401)—PSG2 (OMIM Ref. No. 176391)—PSG5 (OMIM Ref. No. 176394)—PSG4—PSG11 (PSG9; 176398)—tel. The PSG genes are tandemly oriented in a 5-prime to 3-prime direction from telomere to centromere. The CEA subgroup gene CGM11 is located at the telomeric end of the PSG gene cluster, and 6 genes belonging to a third CEA family subgroup, namely CGM13 through CGM18 (later OMIM Ref. No. 109770), are interspersed among the PSG genes.

Full details of the abovementioned studies are described in the following publications, the disclosure of which are hereby incorporated by reference:
Brandriff, B. F.; Gordon, L. A.; Tynan, K. T.; Olsen, A. S.; Mohrenweiser, H. W.; Fertitta, A.; Carrano, A. V.; Trask, B. J.: Order and genomic distances among members of the carcinoembryonic antigen (CEA) gene family determined by fluorescence in situ hybridization. Genomics 12: 773-779, 1992.; and
Teglund, S.; Olsen, A.; Khan, W. N.; Frangsmyr, L.; Hammarstrom, S.: The pregnancy-specific glycoprotein (PSG) gene cluster on human chromosome 19: fine structure of the 11 PSG genes a.

Further studies establishing the function and utilities of PSG1 are found in John Hopkins OMIM database record ID 176390, and in sited publications numbered 2453-2455, 50 and 2456-2466 listed in the bibliography section hereinbelow, which are also hereby incorporated by reference. V-ral Simian Leukemia Viral Oncogene Homolog B (ras related; GTP binding protein) (RALB, Accession NM_{—}002881) is another VGAM76 host target gene. RALB BINDING SITE is HOST TARGET binding site found in the 3′ untranslated region of mRNA encoded by RALB, corresponding to a HOST TARGET binding site such as BINDING SITE I, BINDING SITE II or BINDING SITE III. Table 2 illustrates the complementarity of the nucleotide sequences of RALB BINDING SITE, designated SEQ ID:968, to the nucleotide sequence of VGAM76 RNA, herein designated VGAM RNA, also designated SEQ ID:411.

Another function of VGAM76 is therefore inhibition of V-ral Simian Leukemia Viral Oncogene Homolog B (ras related; GTP binding protein) (RALB, Accession NM_{—}002881). Accordingly, utilities of VGAM76 include diagnosis, prevention and treatment of diseases and clinical conditions associated with RALB. Carcinoembryonic Antigen-related Cell Adhesion Molecule 8 (CEACAM8, Accession NM_{—}001816) is another VGAM76 host target gene. CEACAM8 BINDING SITE is HOST TARGET binding site found in the 3′ untranslated region of mRNA encoded by CEACAM8, corresponding to a HOST TARGET binding site such as BINDING SITE I, BINDING SITE II or BINDING SITE III. Table 2 illustrates the complementarity of the nucleotide sequences of CEACAM8 BINDING SITE, designated SEQ ID:859, to the nucleotide sequence of VGAM76 RNA, herein designated VGAM RNA, also designated SEQ ID:411.

Another function of VGAM76 is therefore inhibition of Carcinoembryonic Antigen-related Cell Adhesion Molecule 8 (CEACAM8, Accession NM_{—}001816). Accordingly, utilities of VGAM76 include diagnosis, prevention and treatment of diseases and clinical conditions associated with CEACAM8. LAT1-3TM (Accession NM_{—}031211) is another VGAM76 host target gene. LAT1-3TM BINDING SITE1 and LAT1-3TM BINDING SITE2 are HOST TARGET binding sites found in untranslated regions of mRNA encoded by LAT1-3TM, corresponding to HOST TARGET binding sites such as BINDING SITE I, BINDING SITE II or BINDING SITE III. Table 2 illustrates the complementarity of the nucleotide sequences of LAT1-3TM BINDING SITE1 and LAT1-3TM BINDING SITE2, designated SEQ ID:2183 and SEQ ID:2827 respectively, to the nucleotide sequence of VGAM76 RNA, herein designated VGAM RNA, also designated SEQ ID:411.

Another function of VGAM76 is therefore inhibition of LAT1-3TM (Accession NM_{—}031211). Accordingly, utilities of VGAM76 include diagnosis, prevention and treatment of diseases and clinical conditions associated with LAT1-3TM. MGC26651 (Accession NM_{—}144642) is another VGAM76 host target gene. MGC26651 BINDING SITE is HOST TARGET binding site found in the 3′ untranslated region of mRNA encoded by MGC26651, corresponding to a HOST TARGET binding site such as BINDING SITE I, BINDING SITE II or BINDING SITE III. Table 2 illustrates the complementarity of the nucleotide sequences of MGC26651 BINDING SITE, designated SEQ ID:2489, to the nucleotide sequence of VGAM76 RNA, herein designated VGAM RNA, also designated SEQ ID:411.

Another function of VGAM76 is therefore inhibition of MGC26651 (Accession NM_{—}144642). Accordingly, utilities of VGAM76 include diagnosis, prevention and treatment of diseases and clinical conditions associated with MGC26651. LOC197423 (Accession XM_{—}085436) is another VGAM76 host target gene. LOC197423 BINDING SITE is HOST TARGET binding site found in the 5′ untranslated region of mRNA encoded by LOC197423, corresponding to a HOST TARGET binding site such as BINDING SITE I, BINDING SITE II or BINDING SITE III. Table 2 illustrates the complementarity of the nucleotide sequences of LOC197423 BINDING SITE, designated SEQ ID:3090, to the nucleotide sequence of VGAM76 RNA, herein designated VGAM RNA, also designated SEQ ID:411.

Another function of VGAM76 is therefore inhibition of LOC197423 (Accession XM_{—}085436). Accordingly, utilities of VGAM76 include diagnosis, prevention and treatment of diseases and clinical conditions associated with LOC197423. LOC220565 (Accession XM_{—}165417) is another VGAM76 host target gene. LOC220565 BINDING SITE is HOST TARGET binding site found in the 5′ untranslated region of mRNA encoded by LOC220565, corresponding to a HOST TARGET binding site such as BINDING SITE I, BINDING SITE II or BINDING SITE III. Table 2 illustrates the complementarity of the nucleotide sequences of LOC220565 BINDING SITE, designated SEQ ID:3495, to the nucleotide sequence of VGAM76 RNA, herein designated VGAM RNA, also designated SEQ ID:411.

Another function of VGAM76 is therefore inhibition of LOC220565 (Accession XM_{—}165417). Accordingly, utilities of VGAM76 include diagnosis, prevention and treatment of diseases and clinical conditions associated with LOC220565. FIG. 1 further provides a conceptual description of a novel bioinformatically detected viral gene of the present invention, referred to here as Viral Genomic Address Messenger 77 (VGAM77) viral gene, which modulates expression of respective host target genes thereof, the function and utility of which host target genes is known in the art.

VGAM77 is a novel bioinformatically detected regulatory, non protein coding, viral micro RNA (miRNA) gene. The method by which VGAM77 was detected is described hereinabove with reference to FIGS. 1-8.

VGAM77 gene, herein designated VGAM GENE, is a viral gene contained in the genome of Vaccinia Virus. VGAM77 host target gene, herein designated VGAM HOST TARGET GENE, is a human gene contained in the human genome.

VGAM77 gene encodes a VGAM77 precursor RNA, herein designated VGAM PRECURSOR RNA. Similar to other miRNA genes, and unlike most ordinary genes, VGAM77 precursor RNA does not encode a protein. A nucleotide sequence identical or highly similar to the nucleotide sequence of VGAM77 precursor RNA is designated SEQ ID:63, and is provided hereinbelow with reference to the sequence listing part. Nucleotide sequence SEQ ID:63 is located at position 17339 relative to the genome of Vaccinia Virus.

VGAM77 precursor RNA folds onto itself, forming VGAM77 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM77 folded precursor RNA into VGAM77 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM77 RNA is designated SEQ ID:412, and is provided hereinbelow with reference to the sequence listing part.

VGAM77 host target gene, herein designated VGAM HOST TARGET GENE, encodes a corresponding messenger RNA, VGAM77 host target RNA, herein designated VGAM HOST TARGET RNA. VGAM77 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM77 RNA, herein designated VGAM RNA, binds complementarily to one or more host target binding sites located in untranslated regions of VGAM77 host target RNA, herein designated VGAM HOST TARGET RNA. This complementary binding is due to the fact that the nucleotide sequence of VGAM77 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM77 RNA, herein designated VGAM RNA, may have a different number of host target binding sites in untranslated regions of a VGAM77 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM77 RNA, herein designated VGAM RNA, to host target binding sites on VGAM77 host target RNA, herein designated VGAM HOST TARGET RNA, such as BINDING SITE I, BINDING SITE II and BINDING SITE III, inhibits translation of VGAM77 host target RNA into VGAM77 host target protein, herein designated VGAM HOST TARGET PROTEIN. VGAM host target protein is therefore outlined by a broken line.

It is appreciated that VGAM77 host target gene, herein designated VGAM HOST TARGET GENE, in fact represents a plurality of VGAM77 host target genes. The mRNA of each one of this plurality of VGAM77 host target genes comprises one or more host target binding sites, each having a nucleotide sequence which is at least partly complementary to VGAM77 RNA, herein designated VGAM RNA, and which when bound by VGAM77 RNA causes inhibition of translation of respective one or more VGAM77 host target proteins.

It is further appreciated by one skilled in the art that the mode of translational inhibition illustrated by FIG. 1 with specific reference to translational inhibition exerted by VGAM77 gene, herein designated VGAM GENE, on one or more VGAM77 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM77 is inhibition of expression of host target genes, as part of a novel viral mechanism of attacking a host. Accordingly, utilities of VGAM77 include diagnosis, prevention and treatment of viral infection by Vaccinia Virus. Specific functions, and accordingly utilities, of VGAM77 correlate with, and may be deduced from, the identity of the host target genes which VGAM77 binds and inhibits, and the function of these host target genes, as elaborated hereinbelow.

Nucleotide sequences of the VGAM77 precursor RNA, herein designated VGAM PRECURSOR RNA, and of the ‘diced’ VGAM77 RNA, herein designated VGAM RNA, and a schematic representation of the secondary folding of VGAM77 folded precursor RNA, herein designated VGAM FOLDED PRECURSOR RNA, of VGAM77 are further described hereinbelow with reference to Table 1.

Nucleotide sequences of host target binding sites, such as BINDING SITE-I, BINDING SITE-II and BINDING SITE-III of FIG. 1, found on VGAM77 host target RNA, and schematic representation of the complementarity of each of these host target binding sites to VGAM77 RNA, herein designated VGAM RNA, are described hereinbelow with reference to Table 2.

As mentioned hereinabove with reference to FIG. 1, a function of VGAM77 gene, herein designated VGAM is inhibition of expression of VGAM77 target genes. It is appreciated that specific functions, and accordingly utilities, of VGAM77 correlate with, and may be deduced from, the identity of the target genes which VGAM77 binds and inhibits, and the function of these target genes, as elaborated hereinbelow.

FLJ13215 (Accession NM_{—}025004) is a VGAM77 host target gene. FLJ13215 BINDING SITE is HOST TARGET binding site found in the 3′ untranslated region of mRNA encoded by FLJ13215, corresponding to a HOST TARGET binding site such as BINDING SITE I, BINDING SITE II or BINDING SITE III. Table 2 illustrates the complementarity of the nucleotide sequences of FLJ13215 BINDING SITE, designated SEQ ID:2126, to the nucleotide sequence of VGAM77 RNA, herein designated VGAM RNA, also designated SEQ ID:412.

A function of VGAM77 is therefore inhibition of FLJ13215 (Accession NM_{—}025004). Accordingly, utilities of VGAM77 include diagnosis, prevention and treatment of diseases and clinical conditions associated with FLJ13215. Protein Tyrosine Phosphatase Type IVA, Member 1 (PTP4A1, Accession NM_{—}003463) is another VGAM77 host target gene. PTP4A1 BINDING SITE is HOST TARGET binding site found in the 3′ untranslated region of mRNA encoded by PTP4A1, corresponding to a HOST TARGET binding site such as BINDING SITE I, BINDING SITE II or BINDING SITE III. Table 2 illustrates the complementarity of the nucleotide sequences of PTP4A1 BINDING SITE, designated SEQ ID:1027, to the nucleotide sequence of VGAM77 RNA, herein designated VGAM RNA, also designated SEQ ID:412.

Another function of VGAM77 is therefore inhibition of Protein Tyrosine Phosphatase Type IVA, Member 1 (PTP4A1, Accession NM_{—}003463). Accordingly, utilities of VGAM77 include diagnosis, prevention and treatment of diseases and clinical conditions associated with PTP4A1. Stromal Cell Derived Factor Receptor 1 (SDFR1, Accession NM_{—}012428) is another VGAM77 host target gene. SDFR1 BINDING SITE1 and SDFR1 BINDING SITE2 are HOST TARGET binding sites found in untranslated regions of mRNA encoded by SDFR1, corresponding to HOST TARGET binding sites such as BINDING SITE I, BINDING SITE II or BINDING SITE III. Table 2 illustrates the complementarity of the nucleotide sequences of SDFR1 BINDING SITE1 and SDFR1 BINDING SITE2, designated SEQ ID: 1430 and SEQ ID:1718 respectively, to the nucleotide sequence of VGAM77 RNA, herein designated VGAM RNA, also designated SEQ ID:412.

Another function of VGAM77 is therefore inhibition of Stromal Cell Derived Factor Receptor 1 (SDFR1, Accession NM_{—}012428). Accordingly, utilities of VGAM77 include diagnosis, prevention and treatment of diseases and clinical conditions associated with SDFR1. FIG. 1 further provides a conceptual description of a novel bioinformatically detected viral gene of the present invention, referred to here as Viral Genomic Address Messenger 78 (VGAM78) viral gene, which modulates expression of respective host target genes thereof, the function and utility of which host target genes is known in the art.

VGAM78 is a novel bioinformatically detected regulatory, non protein coding, viral micro RNA (miRNA) gene. The method by which VGAM78 was detected is described hereinabove with reference to FIGS. 1-8.

VGAM78 gene, herein designated VGAM GENE, is a viral gene contained in the genome of Vaccinia Virus. VGAM78 host target gene, herein designated VGAM HOST TARGET GENE, is a human gene contained in the human genome.

VGAM78 gene encodes a VGAM78 precursor RNA, herein designated VGAM PRECURSOR RNA. Similar to other miRNA genes, and unlike most ordinary genes, VGAM78 precursor RNA does not encode a protein. A nucleotide sequence identical or highly similar to the nucleotide sequence of VGAM78 precursor RNA is designated SEQ ID:64, and is provided hereinbelow with reference to the sequence listing part. Nucleotide sequence SEQ ID:64 is located at position 17430 relative to the genome of Vaccinia Virus.

VGAM78 precursor RNA folds onto itself, forming VGAM78 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM78 folded precursor RNA into VGAM78 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM78 RNA is designated SEQ ID:413, and is provided hereinbelow with reference to the sequence listing part.

VGAM78 host target gene, herein designated VGAM HOST TARGET GENE, encodes a corresponding messenger RNA, VGAM78 host target RNA, herein designated VGAM HOST TARGET RNA. VGAM78 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM78 RNA, herein designated VGAM RNA, binds complementarily to one or more host target binding sites located in untranslated regions of VGAM78 host target RNA, herein designated VGAM HOST TARGET RNA. This complementary binding is due to the fact that the nucleotide sequence of VGAM78 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM78 RNA, herein designated VGAM RNA, may have a different number of host target binding sites in untranslated regions of a VGAM78 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM78 RNA, herein designated VGAM RNA, to host target binding sites on VGAM78 host target RNA, herein designated VGAM HOST TARGET RNA, such as BINDING SITE I, BINDING SITE II and BINDING SITE III, inhibits translation of VGAM78 host target RNA into VGAM78 host target protein, herein designated VGAM HOST TARGET PROTEIN. VGAM host target protein is therefore outlined by a broken line.

It is appreciated that VGAM78 host target gene, herein designated VGAM HOST TARGET GENE, in fact represents a plurality of VGAM78 host target genes. The mRNA of each one of this plurality of VGAM78 host target genes comprises one or more host target binding sites, each having a nucleotide sequence which is at least partly complementary to VGAM78 RNA, herein designated VGAM RNA, and which when bound by VGAM78 RNA causes inhibition of translation of respective one or more VGAM78 host target proteins.

It is further appreciated by one skilled in the art that the mode of translational inhibition illustrated by FIG. 1 with specific reference to translational inhibition exerted by VGAM78 gene, herein designated VGAM GENE, on one or more VGAM78 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM78 is inhibition of expression of host target genes, as part of a novel viral mechanism of attacking a host. Accordingly, utilities of VGAM78 include diagnosis, prevention and treatment of viral infection by Vaccinia Virus. Specific functions, and accordingly utilities, of VGAM78 correlate with, and may be deduced from, the identity of the host target genes which VGAM78 binds and inhibits, and the function of these host target genes, as elaborated hereinbelow.

Nucleotide sequences of the VGAM78 precursor RNA, herein designated VGAM PRECURSOR RNA, and of the ‘diced’ VGAM78 RNA, herein designated VGAM RNA, and a schematic representation of the secondary folding of VGAM78 folded precursor RNA, herein designated VGAM FOLDED PRECURSOR RNA, of VGAM78 are further described hereinbelow with reference to Table 1.

Nucleotide sequences of host target binding sites, such as BINDING SITE-I, BINDING SITE-II and BINDING SITE-III of FIG. 1, found on VGAM78 host target RNA, and schematic representation of the complementarity of each of these host target binding sites to VGAM78 RNA, herein designated VGAM RNA, are described hereinbelow with reference to Table 2.

As mentioned hereinabove with reference to FIG. 1, a function of VGAM78 gene, herein designated VGAM is inhibition of expression of VGAM78 target genes. It is appreciated that specific functions, and accordingly utilities, of VGAM78 correlate with, and may be deduced from, the identity of the target genes which VGAM78 binds and inhibits, and the function of these target genes, as elaborated hereinbelow.

DJ667H12.2 (Accession NM_{—}019605) is a VGAM78 host target gene. DJ667H12.2 BINDING SITE is HOST TARGET binding site found in the 3′ untranslated region of mRNA encoded by DJ667H12.2, corresponding to a HOST TARGET binding site such as BINDING SITE I, BINDING SITE II or BINDING SITE III. Table 2 illustrates the complementarity of the nucleotide sequences of DJ667H12.2 BINDING SITE, designated SEQ ID:1884, to the nucleotide sequence of VGAM78 RNA, herein designated VGAM RNA, also designated SEQ ID:413.

A function of VGAM78 is therefore inhibition of DJ667H12.2 (Accession NM_{—}019605). Accordingly, utilities of VGAM78 include diagnosis, prevention and treatment of diseases and clinical conditions associated with DJ667H12.2. KIAA1799 (Accession XM_{—}059109) is another VGAM78 host target gene. KIAA1799 BINDING SITE is HOST TARGET binding site found in the 3′ untranslated region of mRNA encoded by KIAA1799, corresponding to a HOST TARGET binding site such as BINDING SITE I, BINDING SITE II or BINDING SITE III. Table 2 illustrates the complementarity of the nucleotide sequences of KIAA1799 BINDING SITE, designated SEQ ID:3003, to the nucleotide sequence of VGAM78 RNA, herein designated VGAM RNA, also designated SEQ ID:413.

Another function of VGAM78 is therefore inhibition of KIAA1799 (Accession XM_{—}059109). Accordingly, utilities of VGAM78 include diagnosis, prevention and treatment of diseases and clinical conditions associated with KIAA1799. Protein Phosphatase 1, Regulatory (inhibitor) Subunit 3B (PPP1R3B, Accession NM_{—}024607) is another VGAM78 host target gene. PPP1R3B BINDING SITE is HOST TARGET binding site found in the 3′ untranslated region of mRNA encoded by PPP1R3B, corresponding to a HOST TARGET binding site such as BINDING SITE I, BINDING SITE II or BINDING SITE III. Table 2 illustrates the complementarity of the nucleotide sequences of PPP1R3B BINDING SITE, designated SEQ ID:2071, to the nucleotide sequence of VGAM78 RNA, herein designated VGAM RNA, also designated SEQ ID:413.

Another function of VGAM78 is therefore inhibition of Protein Phosphatase 1, Regulatory (inhibitor) Subunit 3B (PPP1R3B, Accession NM_{—}024607). Accordingly, utilities of VGAM78 include diagnosis, prevention and treatment of diseases and clinical conditions associated with PPP1R3B. LOC144840 (Accession XM_{—}084982) is another VGAM78 host target gene. LOC144840 BINDING SITE is HOST TARGET binding site found in the 3′ untranslated region of mRNA encoded by LOC144840, corresponding to a HOST TARGET binding site such as BINDING SITE I, BINDING SITE II or BINDING SITE III. Table 2 illustrates the complementarity of the nucleotide sequences of LOC144840 BINDING SITE, designated SEQ ID:3071, to the nucleotide sequence of VGAM78 RNA, herein designated VGAM RNA, also designated SEQ ID:413.

Another function of VGAM78 is therefore inhibition of LOC144840 (Accession XM_{—}084982). Accordingly, utilities of VGAM78 include diagnosis, prevention and treatment of diseases and clinical conditions associated with LOC144840. LOC158674 (Accession XM_{—}088638) is another VGAM78 host target gene. LOC158674 BINDING SITE is HOST TARGET binding site found in the 3′ untranslated region of mRNA encoded by LOC158674, corresponding to a HOST TARGET binding site such as BINDING SITE I, BINDING SITE II or BINDING SITE III. Table 2 illustrates the complementarity of the nucleotide sequences of LOC158674 BINDING SITE, designated SEQ ID:3222, to the nucleotide sequence of VGAM78 RNA, herein designated VGAM RNA, also designated SEQ ID:413.

Another function of VGAM78 is therefore inhibition of LOC158674 (Accession XM_{—}088638). Accordingly, utilities of VGAM78 include diagnosis, prevention and treatment of diseases and clinical conditions associated with LOC158674. LOC245757 (Accession XM_{—}165443) is another VGAM78 host target gene. LOC245757 BINDING SITE is HOST TARGET binding site found in the 3′ untranslated region of mRNA encoded by LOC245757, corresponding to a HOST TARGET binding site such as BINDING SITE I, BINDING SITE II or BINDING SITE III. Table 2 illustrates the complementarity of the nucleotide sequences of LOC245757 BINDING SITE, designated SEQ ID:3496, to the nucleotide sequence of VGAM78 RNA, herein designated VGAM RNA, also designated SEQ ID:413.

Another function of VGAM78 is therefore inhibition of LOC245757 (Accession XM_{—}165443). Accordingly, utilities of VGAM78 include diagnosis, prevention and treatment of diseases and clinical conditions associated with LOC245757. LOC81034 (Accession NM_{—}030780) is another VGAM78 host target gene. LOC81034 BINDING SITE is HOST TARGET binding site found in the 3′ untranslated region of mRNA encoded by LOC81034, corresponding to a HOST TARGET binding site such as BINDING SITE I, BINDING SITE II or BINDING SITE III. Table 2 illustrates the complementarity of the nucleotide sequences of LOC81034 BINDING SITE, designated SEQ ID:2164, to the nucleotide sequence of VGAM78 RNA, herein designated VGAM RNA, also designated SEQ ID:413.

Another function of VGAM78 is therefore inhibition of LOC81034 (Accession NM_{—}030780). Accordingly, utilities of VGAM78 include diagnosis, prevention and treatment of diseases and clinical conditions associated with LOC81034. FIG. 1 further provides a conceptual description of a novel bioinformatically detected viral gene of the present invention, referred to here as Viral Genomic Address Messenger 79 (VGAM79) viral gene, which modulates expression of respective host target genes thereof, the function and utility of which host target genes is known in the art.

VGAM79 is a novel bioinformatically detected regulatory, non protein coding, viral micro RNA (miRNA) gene. The method by which VGAM79 was detected is described hereinabove with reference to FIGS. 1-8.

VGAM79 gene, herein designated VGAM GENE, is a viral gene contained in the genome of Vaccinia Virus. VGAM79 host target gene, herein designated VGAM HOST TARGET GENE, is a human gene contained in the human genome.

VGAM79 gene encodes a VGAM79 precursor RNA, herein designated VGAM PRECURSOR RNA. Similar to other miRNA genes, and unlike most ordinary genes, VGAM79 precursor RNA does not encode a protein. A nucleotide sequence identical or highly similar to the nucleotide sequence of VGAM79 precursor RNA is designated SEQ ID:65, and is provided hereinbelow with reference to the sequence listing part. Nucleotide sequence SEQ ID:65 is located at position 17229 relative to the genome of Vaccinia Virus.

VGAM79 precursor RNA folds onto itself, forming VGAM79 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM79 folded precursor RNA into VGAM79 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM79 RNA is designated SEQ ID:414, and is provided hereinbelow with reference to the sequence listing part.

VGAM79 host target gene, herein designated VGAM HOST TARGET GENE, encodes a corresponding messenger RNA, VGAM79 host target RNA, herein designated VGAM HOST TARGET RNA. VGAM79 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM79 RNA, herein designated VGAM RNA, binds complementarily to one or more host target binding sites located in untranslated regions of VGAM79 host target RNA, herein designated VGAM HOST TARGET RNA. This complementary binding is due to the fact that the nucleotide sequence of VGAM79 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM79 RNA, herein designated VGAM RNA, may have a different number of host target binding sites in untranslated regions of a VGAM79 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM79 RNA, herein designated VGAM RNA, to host target binding sites on VGAM79 host target RNA, herein designated VGAM HOST TARGET RNA, such as BINDING SITE I, BINDING SITE II and BINDING SITE III, inhibits translation of VGAM79 host target RNA into VGAM79 host target protein, herein designated VGAM HOST TARGET PROTEIN. VGAM host target protein is therefore outlined by a broken line.

It is appreciated that VGAM79 host target gene, herein designated VGAM HOST TARGET GENE, in fact represents a plurality of VGAM79 host target genes. The mRNA of each one of this plurality of VGAM79 host target genes comprises one or more host target binding sites, each having a nucleotide sequence which is at least partly complementary to VGAM79 RNA, herein designated VGAM RNA, and which when bound by VGAM79 RNA causes inhibition of translation of respective one or more VGAM79 host target proteins.

It is further appreciated by one skilled in the art that the mode of translational inhibition illustrated by FIG. 1 with specific reference to translational inhibition exerted by VGAM79 gene, herein designated VGAM GENE, on one or more VGAM79 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM79 is inhibition of expression of host target genes, as part of a novel viral mechanism of attacking a host. Accordingly, utilities of VGAM79 include diagnosis, prevention and treatment of viral infection by Vaccinia Virus. Specific functions, and accordingly utilities, of VGAM79 correlate with, and may be deduced from, the identity of the host target genes which VGAM79 binds and inhibits, and the function of these host target genes, as elaborated hereinbelow.

Nucleotide sequences of the VGAM79 precursor RNA, herein designated VGAM PRECURSOR RNA, and of the ‘diced’ VGAM79 RNA, herein designated VGAM RNA, and a schematic representation of the secondary folding of VGAM79 folded precursor RNA, herein designated VGAM FOLDED PRECURSOR RNA, of VGAM79 are further described hereinbelow with reference to Table 1.

Nucleotide sequences of host target binding sites, such as BINDING SITE-I, BINDING SITE-II and BINDING SITE-III of FIG. 1, found on VGAM79 host target RNA, and schematic representation of the complementarity of each of these host target binding sites to VGAM79 RNA, herein designated VGAM RNA, are described hereinbelow with reference to Table 2.

As mentioned hereinabove with reference to FIG. 1, a function of VGAM79 gene, herein designated VGAM is inhibition of expression of VGAM79 target genes. It is appreciated that specific functions, and accordingly utilities, of VGAM79 correlate with, and may be deduced from, the identity of the target genes which VGAM79 binds and inhibits, and the function of these target genes, as elaborated hereinbelow.

Dystrobrevin, Beta (DTNB, Accession NM_{—}033147) is a VGAM79 host target gene. DTNB BINDING SITE is HOST TARGET binding site found in the 3′ untranslated region of mRNA encoded by DTNB, corresponding to a HOST TARGET binding site such as BINDING SITE I, BINDING SITE II or BINDING SITE III. Table 2 illustrates the complementarity of the nucleotide sequences of DTNB BINDING SITE, designated SEQ ID:2316, to the nucleotide sequence of VGAM79 RNA, herein designated VGAM RNA, also designated SEQ ID:414.

A function of VGAM79 is therefore inhibition of Dystrobrevin, Beta (DTNB, Accession NM_{—}033147), a gene which is a part of a dystrophin-associated protein complex. Accordingly, utilities of VGAM79 include diagnosis, prevention and treatment of diseases and clinical conditions associated with DTNB. The function of DTNB has been established by previous studies. Dystrophin, a component of muscle that is defective in Duchenne muscular dystrophy (DMD; 310200), binds to a complex of proteins and glycoproteins, the dystrophin-associated protein complex (DPC), which effectively forms a transmembrane link between the extracellular matrix, and the cytoskeleton of the muscle fiber. The DPC can be divided into 3 subcomplexes: the dystroglycan complex, the sarcoglycan complex, and the cytoplasmic complex. The dystroglycan complex consists of 2 proteins, alpha- and beta-dystroglycan (DAG1, 128239; DAG2, 600119), that bind to laminin and dystrophin, respectively. The sarcoglycan complex is composed of 4 transmembrane glycoproteins: alpha-, beta-, gamma-, and delta-sarcoglycan, and a 25-kD protein 25DAP. Mutations in all 4 sarcoglycan genes have been found in patients with different forms of limb-girdle muscular dystrophy. The cytoplasmic component of the DPC is composed of the syntrophin family of related proteins and the dystrophin-related protein, dystrobrevin. Peters et al. (1997) described beta-dystrobrevin. They cloned cDNAs from human liver libraries that encode a 627-amino acid polypeptide with a predicted molecular weight of 71 kD. The protein copurified with the dystrobrevin short form, Dp71. The mammalian dystrobrevin genes encode several protein isoforms that are expressed in different tissues, including brain and muscle. Blake et al. (1998) designated the isoform expressed in muscle as alpha-dystrobrevin and used the designation beta-dystrobrevin for the dystrophin-related protein they found to be abundantly expressed in brain and other tissues but not in muscle. Beta-dystrobrevin is encoded by a 2.5-kb alternatively spliced transcript that is found throughout the brain. In common with dystrophin, beta-dystrobrevin is found in neurons of the cortex and hippocampal formation, but it is not found in the brain microvasculature. In the brain, beta-dystrobrevin coprecipitates with the dystrophin isoforms Dp71 and Dp140. The findings of Blake et al. (1998) indicated that the composition of the dystrophin-associated protein complex in the brain differs from that in muscle. Because beta-dystrobrevin and dystrophin are expressed in similar populations of neurons in the hippocampus and cortex, it is possible that beta-dystrobrevin interacts directly with dystrophin. If this is the case, then beta-dystrobrevin levels may be reduced in DMD patients similar to the reduction in sarcolemmal staining seen with other components of the DPC in dystrophic muscle. The findings may be relevant to the cognitive dysfunction affecting many patients with Duchenne muscular dystrophy.

Full details of the abovementioned studies are described in the following publications, the disclosure of which are hereby incorporated by reference:
Blake, D. J.; Nawrotzki, R.; Loh, N. Y.; Gorecki, D. C.; Davies, K. E.: Beta-dystrobrevin, a member of the dystrophin-related protein family. Proc. Nat. Acad. Sci. 95: 241-246, 1998.; and
Peters, M. F.; O'Brien, K. F.; Sadoulet-Puccio, H. M.; Kunkel, L. M.; Adams, M. E.; Froehner, S. C.: Beta-dystrobrevin, a new member of the dystrophin family: identification, cloning.

Further studies establishing the function and utilities of DTNB are found in John Hopkins OMIM database record ID 602415, and in sited publications numbered 650-232 listed in the bibliography section hereinbelow, which are also hereby incorporated by reference. Nuclear Antigen Sp100 (SP100, Accession NM_{—}003113) is another VGAM79 host target gene. SP100 BINDING SITE is HOST TARGET binding site found in the 3′ untranslated region of mRNA encoded by SP100, corresponding to a HOST TARGET binding site such as BINDING SITE I, BINDING SITE II or BINDING SITE III. Table 2 illustrates the complementarity of the nucleotide sequences of SP100 BINDING SITE, designated SEQ ID:993, to the nucleotide sequence of VGAM79 RNA, herein designated VGAM RNA, also designated SEQ ID:414.

Another function of VGAM79 is therefore inhibition of Nuclear Antigen Sp100 (SP100, Accession NM_{—}003113), a gene which may be involved in transduction of interferon action. Accordingly, utilities of VGAM79 include diagnosis, prevention and treatment of diseases and clinical conditions associated with SP100. The function of SP100 has been established by previous studies. Seeler et al. (1998) showed that SP100 complexes with members of the HP1 family of nonhistone chromosomal proteins (e.g., CBX5, 604478). A variant of SP100, termed SP100B by the authors, contains additional 3-prime sequence encoding a 688-amino acid protein. A splice variant of SP100B, termed SP100-HMG, is joined to an 81-amino acid HMG1 (OMIM Ref. No. 163905)-like peptide by a 14-amino acid bridge. The HMG1-like domain is 87% identical and 93% similar to HMG1. SP100-HMG has the potential to be a DNA-binding protein. All 3 variants, SP100, SP100B, and SP100-HMG, colocalize with HP1 in NBs, suggesting that the N-terminal portion of SP100 is responsible for the interaction. HP1 expression is enhanced when SP100 synthesis is induced by interferon. By Northern blot analysis, Dent et al. (1996) found that SP100B, which they called LYSP100, is expressed only in lymphoid tissues (spleen, tonsil, and thymus), mature B-cell lines, and some T-cell lines, but not in brain, liver, muscle, or nonlymphoid cell lines. They noted that SP100 expression is widespread. By confocal immunofluorescence microscopy, they determined that a minority of the nuclear dots for SP100B overlapped with SP100 and PML, whereas most localized to another class of subnuclear structures, which they termed LANDs (LYSP100-associated nuclear domains), which are morphologically and spatially distinct from PML NBs.

Full details of the abovementioned studies are described in the following publications, the disclosure of which are hereby incorporated by reference:
Seeler, J. S.; Marchio, A.; Sitterlin, D.; Transy, C.; Dejean, A.: Interaction of SP100 with HP1 proteins: a link between the promyelocytic leukemia-associated nuclear bodies and the chromatin compartment. Proc. Nat. Acad. Sci. 95: 7316-7321, 1998.; and
Dent, A. L.; Yewdell, J.; Puvion-Dutilleul, F.; Koken, M. H.; de The, H.; Staudt, L. M.: LYSP100 associated nuclear domains (LANDs): description of a new class of subnuclear structures a.

Further studies establishing the function and utilities of SP100 are found in John Hopkins OMIM database record ID 604585, and in sited publications numbered 1117-111 and 1039 listed in the bibliography section hereinbelow, which are also hereby incorporated by reference. KIAA1229 (Accession XM_{—}030665) is another VGAM79 host target gene. KIAA1229 BINDING SITE is HOST TARGET binding site found in the 3′ untranslated region of mRNA encoded by KIAA1229, corresponding to a HOST TARGET binding site such as BINDING SITE I, BINDING SITE II or BINDING SITE III. Table 2 illustrates the complementarity of the nucleotide sequences of KIAA1229 BINDING SITE, designated SEQ ID:2620, to the nucleotide sequence of VGAM79 RNA, herein designated VGAM RNA, also designated SEQ ID:414.

Another function of VGAM79 is therefore inhibition of KIAA1229 (Accession XM_{—}030665). Accordingly, utilities of VGAM79 include diagnosis, prevention and treatment of diseases and clinical conditions associated with KIAA1229. LOC144776 (Accession XM_{—}084964) is another VGAM79 host target gene. LOC144776 BINDING SITE is HOST TARGET binding site found in the 3′ untranslated region of mRNA encoded by LOC144776, corresponding to a HOST TARGET binding site such as BINDING SITE I, BINDING SITE II or BINDING SITE III. Table 2 illustrates the complementarity of the nucleotide sequences of LOC144776 BINDING SITE, designated SEQ ID:3070, to the nucleotide sequence of VGAM79 RNA, herein designated VGAM RNA, also designated SEQ ID:414.

Another function of VGAM79 is therefore inhibition of LOC144776 (Accession XM_{—}084964). Accordingly, utilities of VGAM79 include diagnosis, prevention and treatment of diseases and clinical conditions associated with LOC144776. LOC199775 (Accession XM_{—}114016) is another VGAM79 host target gene. LOC199775 BINDING SITE is HOST TARGET binding site found in the 5′ untranslated region of mRNA encoded by LOC199775, corresponding to a HOST TARGET binding site such as BINDING SITE I, BINDING SITE II or BINDING SITE III. Table 2 illustrates the complementarity of the nucleotide sequences of LOC199775 BINDING SITE, designated SEQ ID:3432, to the nucleotide sequence of VGAM79 RNA, herein designated VGAM RNA, also designated SEQ ID:414.

Another function of VGAM79 is therefore inhibition of LOC199775 (Accession XM_{—}114016). Accordingly, utilities of VGAM79 include diagnosis, prevention and treatment of diseases and clinical conditions associated with LOC199775. LOC222236 (Accession XM_{—}168636) is another VGAM79 host target gene. LOC222236 BINDING SITE is HOST TARGET binding site found in the 3′ untranslated region of mRNA encoded by LOC222236, corresponding to a HOST TARGET binding site such as BINDING SITE I, BINDING SITE II or BINDING SITE III. Table 2 illustrates the complementarity of the nucleotide sequences of LOC222236 BINDING SITE, designated SEQ ID:3651, to the nucleotide sequence of VGAM79 RNA, herein designated VGAM RNA, also designated SEQ ID:414.

Another function of VGAM79 is therefore inhibition of LOC222236 (Accession XM_{—}168636). Accordingly, utilities of VGAM79 include diagnosis, prevention and treatment of diseases and clinical conditions associated with LOC222236. FIG. 1 further provides a conceptual description of a novel bioinformatically detected viral gene of the present invention, referred to here as Viral Genomic Address Messenger 80 (VGAM80) viral gene, which modulates expression of respective host target genes thereof, the function and utility of which host target genes is known in the art.

VGAM80 is a novel bioinformatically detected regulatory, non protein coding, viral micro RNA (miRNA) gene. The method by which VGAM80 was detected is described hereinabove with reference to FIGS. 1-8.

VGAM80 gene, herein designated VGAM GENE, is a viral gene contained in the genome of Vaccinia Virus. VGAM80 host target gene, herein designated VGAM HOST TARGET GENE, is a human gene contained in the human genome.

VGAM80 gene encodes a VGAM80 precursor RNA, herein designated VGAM PRECURSOR RNA. Similar to other miRNA genes, and unlike most ordinary genes, VGAM80 precursor RNA does not encode a protein. A nucleotide sequence identical or highly similar to the nucleotide sequence of VGAM80 precursor RNA is designated SEQ ID:66, and is provided hereinbelow with reference to the sequence listing part. Nucleotide sequence SEQ ID:66 is located at position 18904 relative to the genome of Vaccinia Virus.

VGAM80 precursor RNA folds onto itself, forming VGAM80 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM80 folded precursor RNA into VGAM80 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM80 RNA is designated SEQ ID:415, and is provided hereinbelow with reference to the sequence listing part.

VGAM80 host target gene, herein designated VGAM HOST TARGET GENE, encodes a corresponding messenger RNA, VGAM80 host target RNA, herein designated VGAM HOST TARGET RNA. VGAM80 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM80 RNA, herein designated VGAM RNA, binds complementarily to one or more host target binding sites located in untranslated regions of VGAM80 host target RNA, herein designated VGAM HOST TARGET RNA. This complementary binding is due to the fact that the nucleotide sequence of VGAM80 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM80 RNA, herein designated VGAM RNA, may have a different number of host target binding sites in untranslated regions of a VGAM80 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM80 RNA, herein designated VGAM RNA, to host target binding sites on VGAM80 host target RNA, herein designated VGAM HOST TARGET RNA, such as BINDING SITE I, BINDING SITE II and BINDING SITE III, inhibits translation of VGAM80 host target RNA into VGAM80 host target protein, herein designated VGAM HOST TARGET PROTEIN. VGAM host target protein is therefore outlined by a broken line.

It is appreciated that VGAM80 host target gene, herein designated VGAM HOST TARGET GENE, in fact represents a plurality of VGAM80 host target genes. The mRNA of each one of this plurality of VGAM80 host target genes comprises one or more host target binding sites, each having a nucleotide sequence which is at least partly complementary to VGAM80 RNA, herein designated VGAM RNA, and which when bound by VGAM80 RNA causes inhibition of translation of respective one or more VGAM80 host target proteins.

It is further appreciated by one skilled in the art that the mode of translational inhibition illustrated by FIG. 1 with specific reference to translational inhibition exerted by VGAM80 gene, herein designated VGAM GENE, on one or more VGAM80 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM80 is inhibition of expression of host target genes, as part of a novel viral mechanism of attacking a host. Accordingly, utilities of VGAM80 include diagnosis, prevention and treatment of viral infection by Vaccinia Virus. Specific functions, and accordingly utilities, of VGAM80 correlate with, and may be deduced from, the identity of the host target genes which VGAM80 binds and inhibits, and the function of these host target genes, as elaborated hereinbelow.

Nucleotide sequences of the VGAM80 precursor RNA, herein designated VGAM PRECURSOR RNA, and of the ‘diced’ VGAM80 RNA, herein designated VGAM RNA, and a schematic representation of the secondary folding of VGAM80 folded precursor RNA, herein designated VGAM FOLDED PRECURSOR RNA, of VGAM80 are further described hereinbelow with reference to Table 1.

Nucleotide sequences of host target binding sites, such as BINDING SITE-I, BINDING SITE-II and BINDING SITE-III of FIG. 1, found on VGAM80 host target RNA, and schematic representation of the complementarity of each of these host target binding sites to VGAM80 RNA, herein designated VGAM RNA, are described hereinbelow with reference to Table 2.

As mentioned hereinabove with reference to FIG. 1, a function of VGAM80 gene, herein designated VGAM is inhibition of expression of VGAM80 target genes. It is appreciated that specific functions, and accordingly utilities, of VGAM80 correlate with, and may be deduced from, the identity of the target genes which VGAM80 binds and inhibits, and the function of these target genes, as elaborated hereinbelow.

Cholinergic Receptor, Nicotinic, Alpha Polypeptide 1 (muscle) (CHRNA1, Accession NM_{—}000079) is a VGAM80 host target gene. CHRNA1 BINDING SITE is HOST TARGET binding site found in the 3′ untranslated region of mRNA encoded by CHRNA1, corresponding to a HOST TARGET binding site such as BINDING SITE I, BINDING SITE II or BINDING SITE III. Table 2 illustrates the complementarity of the nucleotide sequences of CHRNA1 BINDING SITE, designated SEQ ID:701, to the nucleotide sequence of VGAM80 RNA, herein designated VGAM RNA, also designated SEQ ID:415.

A function of VGAM80 is therefore inhibition of Cholinergic Receptor, Nicotinic, Alpha Polypeptide 1 (muscle) (CHRNA1, Accession NM_{—}000079). Accordingly, utilities of VGAM80 include diagnosis, prevention and treatment of diseases and clinical conditions associated with CHRNA1. Tumor Necrosis Factor (ligand) Super family, Member 9 (TNFSF9, Accession NM_{—}003811) is another VGAM80 host target gene. TNFSF9 BINDING SITE is HOST TARGET binding site found in the 3′ untranslated region of mRNA encoded by TNFSF9, corresponding to a HOST TARGET binding site such as BINDING SITE I, BINDING SITE II or BINDING SITE III. Table 2 illustrates the complementarity of the nucleotide sequences of TNFSF9 BINDING SITE, designated SEQ ID: 1062, to the nucleotide sequence of VGAM80 RNA, herein designated VGAM RNA, also designated SEQ ID:415.

Another function of VGAM80 is therefore inhibition of Tumor Necrosis Factor (ligand) Super family, Member 9 (TNFSF9, Accession NM_{—}003811). Accordingly, utilities of VGAM80 include diagnosis, prevention and treatment of diseases and clinical conditions associated with TNFSF9. V-akt Murine Thymoma Viral Oncogene Homolog 3 (protein kinase B, gamma) (AKT3, Accession NM_{—}005465) is another VGAM80 host target gene. AKT3 BINDING SITE is HOST TARGET binding site found in the 3′ untranslated region of mRNA encoded by AKT3, corresponding to a HOST TARGET binding site such as BINDING SITE I, BINDING SITE II or BINDING SITE III. Table 2 illustrates the complementarity of the nucleotide sequences of AKT3 BINDING SITE, designated SEQ ID:1214, to the nucleotide sequence of VGAM80 RNA, herein designated VGAM RNA, also designated SEQ ID:415.

Another function of VGAM80 is therefore inhibition of V-akt Murine Thymoma Viral Oncogene Homolog 3 (protein kinase B, gamma) (AKT3, Accession NM_{—}005465). Accordingly, utilities of VGAM80 include diagnosis, prevention and treatment of diseases and clinical conditions associated with AKT3. FLJ10103 (Accession NM_{—}017996) is another VGAM80 host target gene. FLJ10103 BINDING SITE is HOST TARGET binding site found in the 3′ untranslated region of mRNA encoded by FLJ10103, corresponding to a HOST TARGET binding site such as BINDING SITE I, BINDING SITE II or BINDING SITE III. Table 2 illustrates the complementarity of the nucleotide sequences of FLJ10103 BINDING SITE, designated SEQ ID:1773, to the nucleotide sequence of VGAM80 RNA, herein designated VGAM RNA, also designated SEQ ID:415.

Another function of VGAM80 is therefore inhibition of FLJ10103 (Accession NM_{—}017996). Accordingly, utilities of VGAM80 include diagnosis, prevention and treatment of diseases and clinical conditions associated with FLJ10103. FLJ22582 (Accession NM_{—}025045) is another VGAM80 host target gene. FLJ22582 BINDING SITE is HOST TARGET binding site found in the 3′ untranslated region of mRNA encoded by FLJ22582, corresponding to a HOST TARGET binding site such as BINDING SITE I, BINDING SITE II or BINDING SITE III. Table 2 illustrates the complementarity of the nucleotide sequences of FLJ22582 BINDING SITE, designated SEQ ID:2132, to the nucleotide sequence of VGAM80 RNA, herein designated VGAM RNA, also designated SEQ ID:415.

Another function of VGAM80 is therefore inhibition of FLJ22582 (Accession NM_{—}025045). Accordingly, utilities of VGAM80 include diagnosis, prevention and treatment of diseases and clinical conditions associated with FLJ22582. KIAA1371 (Accession XM_{—}114371) is another VGAM80 host target gene. KIAA1371 BINDING SITE is HOST TARGET binding site found in the 3′ untranslated region of mRNA encoded by KIAA1371, corresponding to a HOST TARGET binding site such as BINDING SITE I, BINDING SITE II or BINDING SITE III. Table 2 illustrates the complementarity of the nucleotide sequences of KIAA1371 BINDING SITE, designated SEQ ID:3454, to the nucleotide sequence of VGAM80 RNA, herein designated VGAM RNA, also designated SEQ ID:415.

Another function of VGAM80 is therefore inhibition of KIAA1371 (Accession XM_{—}114371). Accordingly, utilities of VGAM80 include diagnosis, prevention and treatment of diseases and clinical conditions associated with KIAA1371. Microtubule-actin Crosslinking Factor 1 (MACF1, Accession NM_{—}033024) is another VGAM80 host target gene. MACF1 BINDING SITE1 and MACF1 BINDING SITE2 are HOST TARGET binding sites found in untranslated regions of mRNA encoded by MACF1, corresponding to HOST TARGET binding sites such as BINDING SITE I, BINDING SITE II or BINDING SITE III. Table 2 illustrates the complementarity of the nucleotide sequences of MACF1 BINDING SITE1 and MACF1 BINDING SITE2, designated SEQ ID:2308 and SEQ ID: 1403 respectively, to the nucleotide sequence of VGAM80 RNA, herein designated VGAM RNA, also designated SEQ ID:415.

Another function of VGAM80 is therefore inhibition of Microtubule-actin Crosslinking Factor 1 (MACF1, Accession NM_{—}033024). Accordingly, utilities of VGAM80 include diagnosis, prevention and treatment of diseases and clinical conditions associated with MACF1. Nuclear Receptor Sub family 2, Group C, Member 1 (NR2C1, Accession NM_{—}003297) is another VGAM80 host target gene. NR2C1 BINDING SITE is HOST TARGET binding site found in the 3′ untranslated region of mRNA encoded by NR2C1, corresponding to a HOST TARGET binding site such as BINDING SITE I, BINDING SITE II or BINDING SITE III. Table 2 illustrates the complementarity of the nucleotide sequences of NR2C1 BINDING SITE, designated SEQ ID: 1006, to the nucleotide sequence of VGAM80 RNA, herein designated VGAM RNA, also designated SEQ ID:415.

Another function of VGAM80 is therefore inhibition of Nuclear Receptor Sub family 2, Group C, Member 1 (NR2C1, Accession NM_{—}003297). Accordingly, utilities of VGAM80 include diagnosis, prevention and treatment of diseases and clinical conditions associated with NR2C1. P450RAI-2 (Accession NM_{—}019885) is another VGAM80 host target gene. P450RAI-2 BINDING SITE is HOST TARGET binding site found in the 3′ untranslated region of mRNA encoded by P450RAI-2, corresponding to a HOST TARGET binding site such as BINDING SITE I, BINDING SITE II or BINDING SITE III. Table 2 illustrates the complementarity of the nucleotide sequences of P450RAI-2 BINDING SITE, designated SEQ ID:1887, to the nucleotide sequence of VGAM80 RNA, herein designated VGAM RNA, also designated SEQ ID:415.

Another function of VGAM80 is therefore inhibition of P450RAI-2 (Accession NM_{—}019885). Accordingly, utilities of VGAM80 include diagnosis, prevention and treatment of diseases and clinical conditions associated with P450RAI-2. LOC152715 (Accession XM_{—}087511) is another VGAM80 host target gene. LOC152715 BINDING SITE is HOST TARGET binding site found in the 5′ untranslated region of mRNA encoded by LOC152715, corresponding to a HOST TARGET binding site such as BINDING SITE I, BINDING SITE II or BINDING SITE III. Table 2 illustrates the complementarity of the nucleotide sequences of LOC152715 BINDING SITE, designated SEQ ID:3174, to the nucleotide sequence of VGAM80 RNA, herein designated VGAM RNA, also designated SEQ ID:415.

Another function of VGAM80 is therefore inhibition of LOC152715 (Accession XM_{—}087511). Accordingly, utilities of VGAM80 include diagnosis, prevention and treatment of diseases and clinical conditions associated with LOC152715. LOC222166 (Accession XM_{—}168425) is another VGAM80 host target gene. LOC222166 BINDING SITE is HOST TARGET binding site found in the 3′ untranslated region of mRNA encoded by LOC222166, corresponding to a HOST TARGET binding site such as BINDING SITE I, BINDING SITE II or BINDING SITE III. Table 2 illustrates the complementarity of the nucleotide sequences of LOC222166 BINDING SITE, designated SEQ ID:3634, to the nucleotide sequence of VGAM80 RNA, herein designated VGAM RNA, also designated SEQ ID:415.

Another function of VGAM80 is therefore inhibition of LOC222166 (Accession XM_{—}168425). Accordingly, utilities of VGAM80 include diagnosis, prevention and treatment of diseases and clinical conditions associated with LOC222166. LOC257042 (Accession XM_{—}173039) is another VGAM80 host target gene. LOC257042 BINDING SITE is HOST TARGET binding site found in the 3′ untranslated region of mRNA encoded by LOC257042, corresponding to a HOST TARGET binding site such as BINDING SITE I, BINDING SITE II or BINDING SITE III. Table 2 illustrates the complementarity of the nucleotide sequences of LOC257042 BINDING SITE, designated SEQ ID:3719, to the nucleotide sequence of VGAM80 RNA, herein designated VGAM RNA, also designated SEQ ID:415.

Another function of VGAM80 is therefore inhibition of LOC257042 (Accession XM_{—}173039). Accordingly, utilities of VGAM80 include diagnosis, prevention and treatment of diseases and clinical conditions associated with LOC257042. FIG. 1 further provides a conceptual description of a novel bioinformatically detected viral gene of the present invention, referred to here as Viral Genomic Address Messenger 81 (VGAM81) viral gene, which modulates expression of respective host target genes thereof, the function and utility of which host target genes is known in the art.

VGAM81 is a novel bioinformatically detected regulatory, non protein coding, viral micro RNA (miRNA) gene. The method by which VGAM81 was detected is described hereinabove with reference to FIGS. 1-8.

VGAM81 gene, herein designated VGAM GENE, is a viral gene contained in the genome of Vaccinia Virus. VGAM81 host target gene, herein designated VGAM HOST TARGET GENE, is a human gene contained in the human genome.

VGAM81 gene encodes a VGAM81 precursor RNA, herein designated VGAM PRECURSOR RNA. Similar to other miRNA genes, and unlike most ordinary genes, VGAM81 precursor RNA does not encode a protein. A nucleotide sequence identical or highly similar to the nucleotide sequence of VGAM81 precursor RNA is designated SEQ ID:67, and is provided hereinbelow with reference to the sequence listing part. Nucleotide sequence SEQ ID:67 is located at position 27652 relative to the genome of Vaccinia Virus.

VGAM81 precursor RNA folds onto itself, forming VGAM81 folded precursor RNA, herein designated VGAM FOLDED PRECURSOR RNA, which has a two-dimensional “hairpin structure”. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM81 folded precursor RNA into VGAM81 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM81 RNA is designated SEQ ID:416, and is provided hereinbelow with reference to the sequence listing part.

VGAM81 host target gene, herein designated VGAM HOST TARGET GENE, encodes a corresponding messenger RNA, VGAM81 host target RNA, herein designated VGAM HOST TARGET RNA. VGAM81 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM81 RNA, herein designated VGAM RNA, binds complementarily to one or more host target binding sites located in untranslated regions of VGAM81 host target RNA, herein designated VGAM HOST TARGET RNA. This complementary binding is due to the fact that the nucleotide sequence of VGAM81 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM81 RNA, herein designated VGAM RNA, may have a different number of host target binding sites in untranslated regions of a VGAM81 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM81 RNA, herein designated VGAM RNA, to host target binding sites on VGAM81 host target RNA, herein designated VGAM HOST TARGET RNA, such as BINDING SITE I, BINDING SITE II and BINDING SITE III, inhibits translation of VGAM81 host target RNA into VGAM81 host target protein, herein designated VGAM HOST TARGET PROTEIN. VGAM host target protein is therefore outlined by a broken line.

It is appreciated that VGAM81 host target gene, herein designated VGAM HOST TARGET GENE, in fact represents a plurality of VGAM81 host target genes. The mRNA of each one of this plurality of VGAM81 host target genes comprises one or more host target binding sites, each having a nucleotide sequence which is at least partly complementary to VGAM81 RNA, herein designated VGAM RNA, and which when bound by VGAM81 RNA causes inhibition of translation of respective one or more VGAM81 host target proteins.

It is further appreciated by one skilled in the art that the mode of translational inhibition illustrated by FIG. 1 with specific reference to translational inhibition exerted by VGAM81 gene, herein designated VGAM GENE, on one or more VGAM81 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM81 is inhibition of expression of host target genes, as part of a novel viral mechanism of attacking a host. Accordingly, utilities of VGAM81 include diagnosis, prevention and treatment of viral infection by Vaccinia Virus. Specific functions, and accordingly utilities, of VGAM81 correlate with, and may be deduced from, the identity of the host target genes which VGAM81 binds and inhibits, and the function of these host target genes, as elaborated hereinbelow.

Nucleotide sequences of the VGAM81 precursor RNA, herein designated VGAM PRECURSOR RNA, and of the ‘diced’ VGAM81 RNA, herein designated VGAM RNA, and a schematic representation of the secondary folding of VGAM81 folded precursor RNA, herein designated VGAM FOLDED PRECURSOR RNA, of VGAM81 are further described hereinbelow with reference to Table 1.

Nucleotide sequences of host target binding sites, such as BINDING SITE-I, BINDING SITE-II and BINDING SITE-III of FIG. 1, found on VGAM81 host target RNA, and schematic representation of the complementarity of each of these host target binding sites to VGAM81 RNA, herein designated VGAM RNA, are described hereinbelow with reference to Table 2.

As mentioned hereinabove with reference to FIG. 1, a function of VGAM81 gene, herein designated VGAM is inhibition of expression of VGAM81 target genes. It is appreciated that specific functions, and accordingly utilities, of VGAM81 correlate with, and may be deduced from, the identity of the target genes which VGAM81 binds and inhibits, and the function of these target genes, as elaborated hereinbelow.

Coronin, Actin Binding Protein, 2B (CORO2B, Accession XM_{—}035403) is a VGAM81 host target gene. CORO2B BINDING SITE is HOST TARGET binding site found in the 5′ untranslated region of mRNA encoded by CORO2B, corresponding to a HOST TARGET binding site such as BINDING SITE I, BINDING SITE II or BINDING SITE III. Table 2 illustrates the complementarity of the nucleotide sequences of CORO2B BINDING SITE, designated SEQ ID:2694, to the nucleotide sequence of VGAM81 RNA, herein designated VGAM RNA, also designated SEQ ID:416.

A function of VGAM81 is therefore inhibition of Coronin, Actin Binding Protein, 2B (CORO2B, Accession XM_{—}035403), a gene which may play a role in the reorganization of neuronal actin structure. Accordingly, utilities of VGAM81 include diagnosis, prevention and treatment of diseases and clinical conditions associated with CORO2B. The function of CORO2B has been established by previous studies. The Dictyostelium actin-binding protein coronin accumulates at the leading edges of motile cells and in crown-shaped extensions on the dorsal cell surface. Coronin is involved in cell motility, cytokinesis, and phagocytosis, all of which depend on cytoskeletal rearrangement; see CORO1A (OMIM Ref. No. 605000). By screening a frontal cortex cDNA library with a brain-enriched clone showing similarity to Dictyostelium coronin, Nakamura et al. (1999) isolated a full-length cDNA encoding CORO2B, which they called CLIPINC. The predicted 475-amino acid CORO2B protein has an N-terminal domain containing 5 WD repeats and a succeeding domain with a tendency to form alpha helices. Northern blot analysis detected abundant expression of an approximately 4.0-kb CORO2B transcript in brain, with moderate expression in heart and ovary, and little or no expression in other tissues tested. In contrast, CORO1A is primarily expressed in immune system tissues, and CORO2A (OMIM Ref. No. 602159) is predominantly expressed in colon, prostate, and testis. Immunohistochemical analysis revealed Coro2a expression in mouse cerebral cortex, hippocampus, thalamus, olfactory bulb, and cerebellum, as well as in the inner nuclear layer of embryonic retina and embryonic olfactory bulb. Cosedimentation analysis demonstrated that CORO2B associates with F-actin. Immunofluorescence analysis indicated that CORO2B accumulates at neurite tips, at focal adhesions in association with VCL (OMIM Ref. No. 193065), and along stress fibers.

Full details of the abovementioned studies are described in the following publications, the disclosure of which are hereby incorporated by reference:
Nagase, T.; Ishikawa, K.; Suyama, M.; Kikuno, R.; Hirosawa, M.; Miyajima, N.; Tanaka, A.; Kotani, H.; Nomura, N.; Ohara, O.: Prediction of the coding sequences of unidentified human genes. XIII. The complete sequences of 100 new cDNA clones from brain which code for large proteins in vitro. DNA Res. 6: 63-70, 1999.; and
Nakamura, T.; Takeuchi, K.; Muraoka, S.; Takezoe, H.; Takahashi, N.; Mori, N.: A neurally enriched coronin-like protein, ClipnC, is a novel candidate for an actin cytoskeleton-cortical.

Further studies establishing the function and utilities of CORO2B are found in John Hopkins OMIM database record ID 605002, and in sited publications numbered 1941 listed in the bibliography section hereinbelow, which are also hereby incorporated by reference. Ephrin-B2 (EFNB2, Accession NM_{—}004093) is another VGAM81 host target gene. EFNB2 BINDING SITE is HOST TARGET binding site found in the 3′ untranslated region of mRNA encoded by EFNB2, corresponding to a HOST TARGET binding site such as BINDING SITE I, BINDING SITE II or BINDING SITE III. Table 2 illustrates the complementarity of the nucleotide sequences of EFNB2 BINDING SITE, designated SEQ ID: 1084, to the nucleotide sequence of VGAM81 RNA, herein designated VGAM RNA, also designated SEQ ID:416.

Another function of VGAM81 is therefore inhibition of Ephrin-B2 (EFNB2, Accession NM_{—}004093). Accordingly, utilities of VGAM81 include diagnosis, prevention and treatment of diseases and clinical conditions associated with EFNB2. Glucagon Receptor (GCGR, Accession NM_{—}000160) is another VGAM81 host target gene. GCGR BINDING SITE is HOST TARGET binding site found in the 5′ untranslated region of mRNA encoded by GCGR, corresponding to a HOST TARGET binding site such as BINDING SITE I, BINDING SITE II or BINDING SITE III. Table 2 illustrates the complementarity of the nucleotide sequences of GCGR BINDING SITE, designated SEQ ID:714, to the nucleotide sequence of VGAM81 RNA, herein designated VGAM RNA, also designated SEQ ID:416.

Another function of VGAM81 is therefore inhibition of Glucagon Receptor (GCGR, Accession NM_{—}000160), a gene which controls the rate of hepatic glucose production and insulin secretion. Accordingly, utilities of VGAM81 include diagnosis, prevention and treatment of diseases and clinical conditions associated with GCGR. The function of GCGR has been established by previous studies. Hager et al. (1995) reported the association of a single heterozygous gly-to-ser missense mutation in the glucagon receptor gene with late-onset noninsulin-dependent diabetes mellitus (OMIM Ref. No. 125853). In a pooled set of French and Sardinian patients, the gly40-to-ser mutation showed association with NIDDM (chi square=14.4, P=0.0001). In 18 sibships from 9 French pedigrees, some evidence for linkage to diabetes was found. Receptor binding studies using cultured cells expressing the gly40-to-ser mutation demonstrated that this mutation results in a receptor that binds glucagon with a 3-fold lower affinity compared to the wildtype receptor. The physiologic effects of glucagon (GCG; 138030) are mediated through the glucagon receptor, a 480-amino acid protein that is a member of the super family of receptors characterized by a 7 transmembrane domain structure and by their coupling via GTP-binding proteins (G-proteins) to adenyl cyclase. Menzel et al. (1994) cloned human glucagon receptor cDNA and demonstrated 85% nucleotide and 91% amino acid identity with the rat sequence. By fluorescence in situ hybridization, they localized the GCGR gene to 17q25. An Alu variable poly(A) DNA polymorphism was identified in the gene. Use of the polymorphism in a study of CEPH families showed linkage between the polymorphism and markers localized to distal 17q. The receptor for glucagon-like peptide-1 (GLP1R; 138032), which is derived from the same preproglucagon molecule, has structural similarities but does not bind peptides of related structure and similar function, such as glucagon. Lok et al. (1994) isolated a cDNA encoding a complete functional human glucagon receptor from a liver cDNA library by a combination of polymerase chain reaction and colony hybridization. The cDNA encoded a protein that had 80% identity to rat glucagon receptor, bound (125-1)-labeled glucagon, and transduced a signal leading to increases in the concentration of intracellular cyclic AMP. Southern blot analysis of human DNA suggested the presence of a single GCGR locus. By in situ hybridization, Lok et al. (1994) mapped the GCGR locus to 17q25. Analysis of the genomic sequence showed that the coding region spans over 5.5 kb and is interrupted by 12 introns.

Full details of the abovementioned studies are described in the following publications, the disclosure of which are hereby incorporated by reference:
Hager, J.; Hansen, L.; Vaisse, C.; Vionnet, N.; Philippi, A.; Poller, W.; Velho, G.; Carcassi, C.; Contu, L.; Julier, C.; Cambien, F.; Passa, P.; Lathrop, M.; Kindsvogel, W.; Demenais, F.; Nishimura, E.; Froguel, P.: A missense mutation in the glucagon receptor gene is associated with noninsulin-dependent diabetes mellitus. Nature Genet. 9: 299-304, 1995.; and
Lok, S.; Kuijper, J. L.; Jelinek, L. J.; Kramer, J. M.; Whitmore, T. E.; Sprecher, C. A.; Mathewes, S.; Grant, F. J.; Biggs, S. H.; Rosenberg, G. B.; Sheppard, P. O.; O'Hara, P. J.; Fos.

Further studies establishing the function and utilities of GCGR are found in John Hopkins OMIM database record ID 138033, and in sited publications numbered 776-779 listed in the bibliography section hereinbelow, which are also hereby incorporated by reference. Heme Oxygenase (decycling) 1 (HMOX1, Accession NM_{—}002133) is another VGAM81 host target gene. HMOX1 BINDING SITE is HOST TARGET binding site found in the 3′ untranslated region of mRNA encoded by HMOX1, corresponding to a HOST TARGET binding site such as BINDING SITE I, BINDING SITE II or BINDING SITE III. Table 2 illustrates the complementarity of the nucleotide sequences of HMOX1 BINDING SITE, designated SEQ ID:901, to the nucleotide sequence of VGAM81 RNA, herein designated VGAM RNA, also designated SEQ ID:416.

Another function of VGAM81 is therefore inhibition of Heme Oxygenase (decycling) 1 (HMOX1, Accession NM_{—}002133). Accordingly, utilities of VGAM81 include diagnosis, prevention and treatment of diseases and clinical conditions associated with HMOX1. Heparan Sulfate (glucosamine) 3-O-sulfotransferase 2 (HS3ST2, Accession NM_{—}006043) is another VGAM81 host target gene. HS3ST2 BINDING SITE is HOST TARGET binding site found in the 3′ untranslated region of mRNA encoded by HS3ST2, corresponding to a HOST TARGET binding site such as BINDING SITE I, BINDING SITE II or BINDING SITE III. Table 2 illustrates the complementarity of the nucleotide sequences of HS3ST2 BINDING SITE, designated SEQ ID:1267, to the nucleotide sequence of VGAM81 RNA, herein designated VGAM RNA, also designated SEQ ID:416.

Another function of VGAM81 is therefore inhibition of Heparan Sulfate (glucosamine) 3-O-sulfotransferase 2 (HS3ST2, Accession NM_{—}006043), a gene which plays a role in the generation of heparan sulfate proteoglycan. Accordingly, utilities of VGAM81 include diagnosis, prevention and treatment of diseases and clinical conditions associated with HS3ST2. The function of HS3ST2 has been established by previous studies. By searching an EST database for sequences related to the sulfotransferase domain of 30ST1, Shworak et al. (1999) identified partial cDNAs from the 30ST2, 30ST3A1 (OMIM Ref. No. 604057), and 30ST4 (OMIM Ref. No. 604059) genes. They used the partial cDNAs as probes and recovered additional clones corresponding to these genes and to 30ST3B1 (OMIM Ref. No. 604058). The 30ST2 gene encodes a predicted 367-amino acid protein that, like 30ST3A1 and 30ST3B1, is a predicted type II integral membrane protein. These 3 enzymes contain a positively charged N-terminal domain, a transmembrane domain, a region termed the SPLAG domain because it is rich in serine, proline, leucine, alanine, and glycine, and a C-terminal putative sulfotransferase domain. Although they share a similar regional structure, the only significant sequence homology between these 30ST proteins occurs in the sulfotransferase domains. Northern blot analysis revealed that the 30ST2 and 30ST4 genes were expressed predominantly in brain, while the 30ST3 gene exhibited more widespread expression. In a companion paper, Liu et al. (1999) demonstrated that the 30ST1, 30ST2, and 30ST3 isoforms each generate unique 3-O-sulfated structures. Shworak et al. (1999) stated that the isoforms with different sulfotransferase domains differentially place the rare 3-O-sulfate group in distinct sequence contexts, presumably to regulate discrete biologic activities. This capacity of the sulfotransferase domain to generate distinct sequences may in turn be modulated by the unique N-terminal domains of the proteins. By inclusion within mapped clones, Shworak et al. (1999) mapped the 30ST2 gene to 16p12, near the 30ST4 gene at 16p11.2. Using interspecific backcross analysis, they mapped the mouse 3Ost2 and 3Ost4 genes to the distal region of chromosome 7, in a region sharing homology of synteny with human chromosome 16p.

Full details of the abovementioned studies are described in the following publications, the disclosure of which are hereby incorporated by reference:
Liu, J.; Shworak, N. W.; Sinay, P.; Schwartz, J. J.; Zhang, L.; Fritze, L. M.; Rosenberg, R. D.: Expression of heparan sulfate D-glucosaminyl 3-O-sulfotransferase isoforms reveals novel substrate specificities. J. Biol. Chem. 274: 5185-5192, 1999.; and
Shworak, N. W.; Liu, J.; Petros, L. M.; Zhang, L.; Kobayashi, M.; Copeland, N. G.; Jenkins, N. A.; Rosenberg, R. D.: Multiple isoforms of heparan sulfate D-glucosaminyl 3-O-sulfotransf.

Further studies establishing the function and utilities of HS3ST2 are found in John Hopkins OMIM database record ID 604056, and in sited publications numbered 1148-1149 listed in the bibliography section hereinbelow, which are also hereby incorporated by reference. Pim-2 Oncogene (PIM2, Accession XM_{—}010208) is another VGAM81 host target gene. PIM2 BINDING SITE is HOST TARGET binding site found in the 3′ untranslated region of mRNA encoded by PIM2, corresponding to a HOST TARGET binding site such as BINDING SITE I, BINDING SITE II or BINDING SITE III. Table 2 illustrates the complementarity of the nucleotide sequences of PIM2 BINDING SITE, designated SEQ ID:2550, to the nucleotide sequence of VGAM81 RNA, herein designated VGAM RNA, also designated SEQ ID:416.

Another function of VGAM81 is therefore inhibition of Pim-2 Oncogene (PIM2, Accession XM_{—}010208). Accordingly, utilities of VGAM81 include diagnosis, prevention and treatment of diseases and clinical conditions associated with PIM2. Phosphatidylinositol-4-phosphate 5-kinase, Type I, Beta (PIP5K1B, Accession NM_{—}003558) is another VGAM81 host target gene. PIP5K1B BINDING SITE is HOST TARGET binding site found in the 5′ untranslated region of mRNA encoded by PIP5K1B, corresponding to a HOST TARGET binding site such as BINDING SITE I, BINDING SITE II or BINDING SITE III. Table 2 illustrates the complementarity of the nucleotide sequences of PIP5K1B BINDING SITE, designated SEQ ID: 1034, to the nucleotide sequence of VGAM81 RNA, herein designated VGAM RNA, also designated SEQ ID:416.

Another function of VGAM81 is therefore inhibition of Phosphatidylinositol-4-phosphate 5-kinase, Type I, Beta (PIP5K1B, Accession NM_{—}003558), a gene which catalyses the phosphorylation of phosphatidylinositol-4-phosphate to form phosphatidylinositol-4,5-biphosphate. Accordingly, utilities of VGAM81 include diagnosis, prevention and treatment of diseases and clinical conditions associated with PIP5K1B. The function of PIP5K1B has been established by previous studies. Carvajal et al. (1995) reported the isolation of a gene from the region of the genome associated with Friedreich ataxia (FRDA; 229300). Expression was found to be complex, with multiple transcripts detected in a variety of tissues and evidence of alternative splicing and developmental control. The predicted amino acid sequence for the 2.7-kb transcript showed a marked homology to the deduced amino acid sequence of the MSS4 protein of Saccharomyces cerevisiae, which had been proposed to function in the phosphoinositide cycle, thus suggesting a potential role for the human homolog in signal transduction. Although no evidence of mutation was detected in the transcript, the sequence (which they designated STM7.1) represented only one of the shorter alternatively spliced species identified by Northern analysis and direct sequencing. Carvajal et al. (1996) reported that the X25 (frataxin-encoding) gene described by Campuzano et al. (1996) and shown to be associated with mutations in FRDA patients comprises part of a gene that they had previously identified and named STM7. They reported that the transcription of both STM7 and X25 occurs from the centromere toward the telomere, that the reported sequences of STM7 and X25 did not represent a full-length transcript, that multiple transcripts for each of these genes are present in Northern blots, and that several of these transcripts are of similar size. Carvajal et al. (1996) also reported that less than 10 kb separates the CpG island identified in the X25/exon 1 from the 3-prime end of STM7/exon 16. They further demonstrated that the recombinant protein corresponding to the STM7.1 transcript has phosphatidylinositol-4-phosphate 5-kinase activity. See 606829 for further discussion of the relationship between STM7 and FRDA.

Full details of the abovementioned studies are described in the following publications, the disclosure of which are hereby incorporated by reference:
Campuzano, V.; Montermini, L.; Molto, M. D.; Pianese, L.; Cossee, M.; Cavalcanti, F.; Monros, E.; Rodius, F.; Duclos, F.; Monticelli, A.; Zara, F.; Canizares, J.; Koutnikova, H.; Bidichandani, S. I.; Gellera, C.; Brice, A.; Trouillas, P.; De Michele, G.; Filla, A.; De Frutos, R.; Palau, F.; Patel, P. I.; Di Donato, S.; Mandel, J.-L.; Cocozza, S.; Koenig, M.; Pandolfo, M.: Friedreich's ataxia: autosomal recessive disease caused by an intronic GAA triplet repeat expansion. Science 271: 1423-1427, 1996.; and
Carvajal, J. J.; Pook, M. A.; dos Santos, M.; Doudney, K.; Hillermann, R.; Minogue, S.; Williamson, R.; Hsuan, J. J.; Chamberlain, S.: The Friedreich's ataxia gene encodes a novel phos.

Further studies establishing the function and utilities of PIP5K1B are found in John Hopkins OMIM database record ID 602745, and in sited publications numbered 1881-576 listed in the bibliography section hereinbelow, which are also hereby incorporated by reference. RNA Binding Motif Protein 3 (RBM3, Accession XM_{—}047024) is another VGAM81 host target gene. RBM3 BINDING SITE is HOST TARGET binding site found in the 3′ untranslated region of mRNA encoded by RBM3, corresponding to a HOST TARGET binding site such as BINDING SITE I, BINDING SITE II or BINDING SITE III. Table 2 illustrates the complementarity of the nucleotide sequences of RBM3 BINDING SITE, designated SEQ ID:2887, to the nucleotide sequence of VGAM81 RNA, herein designated VGAM RNA, also designated SEQ ID:416.

Another function of VGAM81 is therefore inhibition of RNA Binding Motif Protein 3 (RBM3, Accession XM_{—}047024). Accordingly, utilities of VGAM81 include diagnosis, prevention and treatment of diseases and clinical conditions associated with RBM3. SMAC (Accession NM_{—}019887) is another VGAM81 host target gene. SMAC BINDING SITE1 through SMAC BINDING SITE3 are HOST TARGET binding sites found in untranslated regions of mRNA encoded by SMAC, corresponding to HOST TARGET binding sites such as BINDING SITE I, BINDING SITE II or BINDING SITE III. Table 2 illustrates the complementarity of the nucleotide sequences of SMAC BINDING SITE1 through SMAC BINDING SITE3, designated SEQ ID:1889, SEQ ID:2459 and SEQ ID:2460 respectively, to the nucleotide sequence of VGAM81 RNA, herein designated VGAM RNA, also designated SEQ ID:416.

Another function of VGAM81 is therefore inhibition of SMAC (Accession NM_{—}019887), a gene which promotes apoptosis via caspase activation. Accordingly, utilities of VGAM81 include diagnosis, prevention and treatment of diseases and clinical conditions associated with SMAC. The function of SMAC has been established by previous studies. Verhagen et al. (2000) identified the murine homolog of SMAC, which they called DIABLO (direct IAP-binding protein with low pI). They showed that DIABLO can bind mammalian IAP homolog A (MIHA, or API3) and can also interact with MIHB (API1; 601712), MIHC (API2; 601721), and OpIAP, the baculoviral IAP. Immunoprecipitation and Western blot analysis indicated that the N-terminally processed, IAP-interacting form of DIABLO is concentrated in membrane fractions in healthy cells but is released into the MIHA-containing cytosolic fractions upon ultraviolet (UV) irradiation. Since transfection of cells with DIABLO was able to counter the protection afforded by MIHA against UV irradiation, the authors suggested that DIABLO may promote apoptosis by binding to IAPs and preventing them from inhibiting caspases. Chai et al. (2000) showed that SMAC/DIABLO promotes not only the proteolytic activation of procaspase-3, but also the enzymatic activity of mature caspase-3, both of which depend upon its ability to interact physically with IAPs. Animal model experiments lend further support to the function of SMAC. Okada et al. (2002) generated Diablo-deficient mice by homologous recombination. Western blot analysis confirmed the null mutation. The mice were fertile and appeared grossly normal at more than 1 year of age, and histologic analysis failed to detect any abnormalities. In vitro analysis indicated an inhibition of procaspase-3 (CASP3; 600636) cleavage in Diablo −/− cell lysates, but all types of Diablo −/− cells tested responded normally to a number of apoptotic stimuli. Fas (OMIM Ref. No. 134637)-mediated apoptosis in liver was also normal in vivo in these mice. The authors concluded that a redundant molecule, possibly Omi (PRSS25; 606441), or molecules are capable of compensating for the loss of Diablo function. Alternatively, they suggested that Diablo may only regulate programmed cell death in specific situations or tissues not yet identified.

It is appreciated that the abovementioned animal model for SMAC is acknowledged by those skilled in the art as a scientifically valid animal model, as can be further appreciated from the publications sited hereinbelow.

Full details of the abovementioned studies are described in the following publications, the disclosure of which are hereby incorporated by reference:
Okada, H.; Suh, W.-K.; Jin, J.; Woo, M.; Du, C.; Elia, A.; Duncan, G. S.; Wakeham, A.; Itie, A.; Lowe, S. W.; Wang, X.; Mak, T. W.: Generation and characterization of Smac/DIABLO-deficient mice. Molec. Cell. Biol. 22: 3509-3517, 2002.; and
Verhagen, A. M.; Ekert, P. G.; Pakusch, M.; Silke, J.; Connolly, L. M.; Reid, G. E.; Moritz, R. L.; Simpson, R. J.; Vaux, D. L.: Identification of DIABLO, a mammalian protein that promote.

Further studies establishing the function and utilities of SMAC are found in John Hopkins OMIM database record ID 605219, and in sited publications numbered 1602-1603, 2065-1605, 206 and 1997 listed in the bibliography section hereinbelow, which are also hereby incorporated by reference. Zinc Finger Protein 10 (KOX 1) (ZNF10, Accession NM_{—}015394) is another VGAM81 host target gene. ZNF10 BINDING SITE is HOST TARGET binding site found in the 5′ untranslated region of mRNA encoded by ZNF10, corresponding to a HOST TARGET binding site such as BINDING SITE I, BINDING SITE II or BINDING SITE III. Table 2 illustrates the complementarity of the nucleotide sequences of ZNF10 BINDING SITE, designated SEQ ID: 1626, to the nucleotide sequence of VGAM81 RNA, herein designated VGAM RNA, also designated SEQ ID:416.

Another function of VGAM81 is therefore inhibition of Zinc Finger Protein 10 (KOX 1) (ZNF10, Accession NM_{—}015394), a gene which may function as a transcriptional regulator. Accordingly, utilities of VGAM81 include diagnosis, prevention and treatment of diseases and clinical conditions associated with ZNF10. The function of ZNF10 has been established by previous studies. In the course of mapping 27 nonoverlapping zinc finger cDNAs from human T cells by analysis of somatic cell hybrids, Huebner et al. (1991) mapped zinc finger protein-10 (KOX1) to 12q13-qter, probably clustered with zinc finger protein-26 (OMIM Ref. No. 194537). Rousseau-Merck et al. (1993) also mapped the KOX1 (ZNF10) gene to 12q24.33 and demonstrated that it and KOX20 (ZNF26) are located within a pulsed field gel electrophoresis fragment less than 300 kb long. The mapping was done by a combination of somatic cell hybridization and in situ hybridization. Since ZNF26 has been mapped to 12q24.33 by in situ hybridization, this also must be the localization of ZNF10.

Full details of the abovementioned studies are described in the following publications, the disclosure of which are hereby incorporated by reference:
Huebner, K.; Druck, T.; Croce, C. M.; Thiesen, H. J.: Twenty-seven nonoverlapping zinc finger cDNAs from human T cells map to nine different chromosomes with apparent clustering. Am. J. Hum. Genet. 48: 726-740, 1991.; and
Rousseau-Merck, M.-F.; Hillion, J.; Jonveaux, P.; Couillin, P.; Seite, P.; Thiesen, H.-J.; Berger, R.: Chromosomal localization of 9 KOX zinc finger genes: physical linkages suggest cluste.

Further studies establishing the function and utilities of ZNF10 are found in John Hopkins OMIM database record ID 194538, and in sited publications numbered 2268-2269 listed in the bibliography section hereinbelow, which are also hereby incorporated by reference. ATP-binding Cassette, Sub-family A (ABC1), Member 10 (ABCA10, Accession NM_{—}080282) is another VGAM81 host target gene. ABCA10 BINDING SITE is HOST TARGET binding site found in the 5′ untranslated region of mRNA encoded by ABCA10, corresponding to a HOST TARGET binding site such as BINDING SITE I, BINDING SITE II or BINDING SITE III. Table 2 illustrates the complementarity of the nucleotide sequences of ABCA10 BINDING SITE, designated SEQ ID:2373, to the nucleotide sequence of VGAM81 RNA, herein designated VGAM RNA, also designated SEQ ID:416.

Another function of VGAM81 is therefore inhibition of ATP-binding Cassette, Sub-family A (ABC1), Member 10 (ABCA10, Accession NM_{—}080282). Accordingly, utilities of VGAM81 include diagnosis, prevention and treatment of diseases and clinical conditions associated with ABCA10. H2A Histone Family, Member J (H2AFJ, Accession NM_{—}018267) is another VGAM81 host target gene. H2AFJ BINDING SITE is HOST TARGET binding site found in the 3′ untranslated region of mRNA encoded by H2AFJ, corresponding to a HOST TARGET binding site such as BINDING SITE I, BINDING SITE II or BINDING SITE III. Table 2 illustrates the complementarity of the nucleotide sequences of H2AFJ BINDING SITE, designated SEQ ID:1807, to the nucleotide sequence of VGAM81 RNA, herein designated VGAM RNA, also designated SEQ ID:416.

Another function of VGAM81 is therefore inhibition of H2A Histone Family, Member J (H2AFJ, Accession NM_{—}018267). Accordingly, utilities of VGAM81 include diagnosis, prevention and treatment of diseases and clinical conditions associated with H2AFJ. HSH2 (Accession NM_{—}032855) is another VGAM81 host target gene. HSH2 BINDING SITE is HOST TARGET binding site found in the 3′ untranslated region of mRNA encoded by HSH2, corresponding to a HOST TARGET binding site such as BINDING SITE I, BINDING SITE II or BINDING SITE III. Table 2 illustrates the complementarity of the nucleotide sequences of HSH2 BINDING SITE, designated SEQ ID:2289, to the nucleotide sequence of VGAM81 RNA, herein designated VGAM RNA, also designated SEQ ID:416.

Another function of VGAM81 is therefore inhibition of HSH2 (Accession NM_{—}032855). Accordingly, utilities of VGAM81 include diagnosis, prevention and treatment of diseases and clinical conditions associated with HSH2. KIAA1024 (Accession XM_{—}044580) is another VGAM81 host target gene. KIAA1024 BINDING SITE is HOST TARGET binding site found in the 3′ untranslated region of mRNA encoded by KIAA1024, corresponding to a HOST TARGET binding site such as BINDING SITE I, BINDING SITE II or BINDING SITE III. Table 2 illustrates the complementarity of the nucleotide sequences of KIAA1024 BINDING SITE, designated SEQ ID:2841, to the nucleotide sequence of VGAM81 RNA, herein designated VGAM RNA, also designated SEQ ID:416.

Another function of VGAM81 is therefore inhibition of KIAA1024 (Accession XM_{—}044580). Accordingly, utilities of VGAM81 include diagnosis, prevention and treatment of diseases and clinical conditions associated with KIAA1024. KIAA1399 (Accession XM_{—}046685) is another VGAM81 host target gene. KIAA1399 BINDING SITE is HOST TARGET binding site found in the 3′ untranslated region of mRNA encoded by KIAA1399, corresponding to a HOST TARGET binding site such as BINDING SITE I, BINDING SITE II or BINDING SITE III. Table 2 illustrates the complementarity of the nucleotide sequences of KIAA1399 BINDING SITE, designated SEQ ID:2881, to the nucleotide sequence of VGAM81 RNA, herein designated VGAM RNA, also designated SEQ ID:416.

Another function of VGAM81 is therefore inhibition of KIAA1399 (Accession XM_{—}046685). Accordingly, utilities of VGAM81 include diagnosis, prevention and treatment of diseases and clinical conditions associated with KIAA1399. MIC2 Like 1 (MIC2Li, Accession NM_{—}031462) is another VGAM81 host target gene. MIC2L1 BINDING SITE is HOST TARGET binding site found in the 3′ untranslated region of mRNA encoded by MIC2Li, corresponding to a HOST TARGET binding site such as BINDING SITE I, BINDING SITE II or BINDING SITE III. Table 2 illustrates the complementarity of the nucleotide sequences of MIC2L1 BINDING SITE, designated SEQ ID:2205, to the nucleotide sequence of VGAM81 RNA, herein designated VGAM RNA, also designated SEQ ID:416.

Another function of VGAM81 is therefore inhibition of MIC2 Like 1 (MIC2Li, Accession NM_{—}031462). Accordingly, utilities of VGAM81 include diagnosis, prevention and treatment of diseases and clinical conditions associated with MIC2L1. Ras Protein-specific Guanine Nucleotide-releasing Factor 2 (RASGRF2, Accession XM_{—}027943) is another VGAM81 host target gene. RAS-GRF2 BINDING SITE is HOST TARGET binding site found in the 3′ untranslated region of mRNA encoded by RASGRF2, corresponding to a HOST TARGET binding site such as BINDING SITE I, BINDING SITE II or BINDING SITE III. Table 2 illustrates the complementarity of the nucleotide sequences of RASGRF2 BINDING SITE, designated SEQ ID:2590, to the nucleotide sequence of VGAM81 RNA, herein designated VGAM RNA, also designated SEQ ID:416.

Another function of VGAM81 is therefore inhibition of Ras Protein-specific Guanine Nucleotide-releasing Factor 2 (RASGRF2, Accession XM_{—}027943). Accordingly, utilities of VGAM81 include diagnosis, prevention and treatment of diseases and clinical conditions associated with RASGRF2. Tight Junction Protein 2 (zona occludens 2) (TJP2, Accession XM_{—}005446) is another VGAM81 host target gene. TJP2 BINDING SITE is HOST TARGET binding site found in the 3′ untranslated region of mRNA encoded by TJP2, corresponding to a HOST TARGET binding site such as BINDING SITE I, BINDING SITE II or BINDING SITE III. Table 2 illustrates the complementarity of the nucleotide sequences of TJP2 BINDING SITE, designated SEQ ID:2534, to the nucleotide sequence of VGAM81 RNA, herein designated VGAM RNA, also designated SEQ ID:416.

Another function of VGAM81 is therefore inhibition of Tight Junction Protein 2 (zona occludens 2) (TJP2, Accession XM_{—}005446). Accordingly, utilities of VGAM81 include diagnosis, prevention and treatment of diseases and clinical conditions associated with TJP2. LOC202020 (Accession XM_{—}114419) is another VGAM81 host target gene. LOC202020 BINDING SITE is HOST TARGET binding site found in the 3′ untranslated region of mRNA encoded by LOC202020, corresponding to a HOST TARGET binding site such as BINDING SITE I, BINDING SITE II or BINDING SITE III. Table 2 illustrates the complementarity of the nucleotide sequences of LOC202020 BINDING SITE, designated SEQ ID:3459, to the nucleotide sequence of VGAM81 RNA, herein designated VGAM RNA, also designated SEQ ID:416.

Another function of VGAM81 is therefore inhibition of LOC202020 (Accession XM_{—}114419). Accordingly, utilities of VGAM81 include diagnosis, prevention and treatment of diseases and clinical conditions associated with LOC202020. LOC220766 (Accession XM_{—}165471) is another VGAM81 host target gene. LOC220766 BINDING SITE is HOST TARGET binding site found in the 5′ untranslated region of mRNA encoded by LOC220766, corresponding to a HOST TARGET binding site such as BINDING SITE I, BINDING SITE II or BINDING SITE III. Table 2 illustrates the complementarity of the nucleotide sequences of LOC220766 BINDING SITE, designated SEQ ID:3499, to the nucleotide sequence of VGAM81 RNA, herein designated VGAM RNA, also designated SEQ ID:416.

Another function of VGAM81 is therefore inhibition of LOC220766 (Accession XM_{—}165471). Accordingly, utilities of VGAM81 include diagnosis, prevention and treatment of diseases and clinical conditions associated with LOC220766. LOC256277 (Accession XM_{—}170644) is another VGAM81 host target gene. LOC256277 BINDING SITE is HOST TARGET binding site found in the 3′ untranslated region of mRNA encoded by LOC256277, corresponding to a HOST TARGET binding site such as BINDING SITE I, BINDING SITE II or BINDING SITE III. Table 2 illustrates the complementarity of the nucleotide sequences of LOC256277 BINDING SITE, designated SEQ ID:3664, to the nucleotide sequence of VGAM81 RNA, herein designated VGAM RNA, also designated SEQ ID:416.

Another function of VGAM81 is therefore inhibition of LOC256277 (Accession XM_{—}170644). Accordingly, utilities of VGAM81 include diagnosis, prevention and treatment of diseases and clinical conditions associated with LOC256277. LOC256337 (Accession XM_{—}170643) is another VGAM81 host target gene. LOC256337 BINDING SITE is HOST TARGET binding site found in the 3′ untranslated region of mRNA encoded by LOC256337, corresponding to a HOST TARGET binding site such as BINDING SITE I, BINDING SITE II or BINDING SITE III. Table 2 illustrates the complementarity of the nucleotide sequences of LOC256337 BINDING SITE, designated SEQ ID:3663, to the nucleotide sequence of VGAM81 RNA, herein designated VGAM RNA, also designated SEQ ID:416.

Another function of VGAM81 is therefore inhibition of LOC256337 (Accession XM_{—}170643). Accordingly, utilities of VGAM81 include diagnosis, prevention and treatment of diseases and clinical conditions associated with LOC256337. FIG. 1 further provides a conceptual description of a novel bioinformatically detected viral gene of the present invention, referred to here as Viral Genomic Address Messenger 82 (VGAM82) viral gene, which modulates expression of respective host target genes thereof, the function and utility of which host target genes is known in the art.

VGAM82 is a novel bioinformatically detected regulatory, non protein coding, viral micro RNA (miRNA) gene. The method by which VGAM82 was detected is described hereinabove with reference to FIGS. 1-8.

VGAM82 gene, herein designated VGAM GENE, is a viral gene contained in the genome of Vaccinia Virus. VGAM82 host target gene, herein designated VGAM HOST TARGET GENE, is a human gene contained in the human genome.

VGAM82 gene encodes a VGAM82 precursor RNA, herein designated VGAM PRECURSOR RNA. Similar to other miRNA genes, and unlike most ordinary genes, VGAM82 precursor RNA does not encode a protein. A nucleotide sequence identical or highly similar to the nucleotide sequence of VGAM82 precursor RNA is designated SEQ ID:68, and is provided hereinbelow with reference to the sequence listing part. Nucleotide sequence SEQ ID:68 is located at position 24541 relative to the genome of Vaccinia Virus.

VGAM82 precursor RNA folds onto itself, forming VGAM82 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM82 folded precursor RNA into VGAM82 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM82 RNA is designated SEQ ID:417, and is provided hereinbelow with reference to the sequence listing part.

VGAM82 host target gene, herein designated VGAM HOST TARGET GENE, encodes a corresponding messenger RNA, VGAM82 host target RNA, herein designated VGAM HOST TARGET RNA. VGAM82 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM82 RNA, herein designated VGAM RNA, binds complementarily to one or more host target binding sites located in untranslated regions of VGAM82 host target RNA, herein designated VGAM HOST TARGET RNA. This complementary binding is due to the fact that the nucleotide sequence of VGAM82 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM82 RNA, herein designated VGAM RNA, may have a different number of host target binding sites in untranslated regions of a VGAM82 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM82 RNA, herein designated VGAM RNA, to host target binding sites on VGAM82 host target RNA, herein designated VGAM HOST TARGET RNA, such as BINDING SITE I, BINDING SITE II and BINDING SITE III, inhibits translation of VGAM82 host target RNA into VGAM82 host target protein, herein designated VGAM HOST TARGET PROTEIN. VGAM host target protein is therefore outlined by a broken line.

It is appreciated that VGAM82 host target gene, herein designated VGAM HOST TARGET GENE, in fact represents a plurality of VGAM82 host target genes. The mRNA of each one of this plurality of VGAM82 host target genes comprises one or more host target binding sites, each having a nucleotide sequence which is at least partly complementary to VGAM82 RNA, herein designated VGAM RNA, and which when bound by VGAM82 RNA causes inhibition of translation of respective one or more VGAM82 host target proteins.

It is further appreciated by one skilled in the art that the mode of translational inhibition illustrated by FIG. 1 with specific reference to translational inhibition exerted by VGAM82 gene, herein designated VGAM GENE, on one or more VGAM82 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM82 is inhibition of expression of host target genes, as part of a novel viral mechanism of attacking a host. Accordingly, utilities of VGAM82 include diagnosis, prevention and treatment of viral infection by Vaccinia Virus. Specific functions, and accordingly utilities, of VGAM82 correlate with, and may be deduced from, the identity of the host target genes which VGAM82 binds and inhibits, and the function of these host target genes, as elaborated hereinbelow.

Nucleotide sequences of the VGAM82 precursor RNA, herein designated VGAM PRECURSOR RNA, and of the ‘diced’ VGAM82 RNA, herein designated VGAM RNA, and a schematic representation of the secondary folding of VGAM82 folded precursor RNA, herein designated VGAM FOLDED PRECURSOR RNA, of VGAM82 are further described hereinbelow with reference to Table 1.

Nucleotide sequences of host target binding sites, such as BINDING SITE-I, BINDING SITE-II and BINDING SITE-III of FIG. 1, found on VGAM82 host target RNA, and schematic representation of the complementarity of each of these host target binding sites to VGAM82 RNA, herein designated VGAM RNA, are described hereinbelow with reference to Table 2.

As mentioned hereinabove with reference to FIG. 1, a function of VGAM82 gene, herein designated VGAM is inhibition of expression of VGAM82 target genes. It is appreciated that specific functions, and accordingly utilities, of VGAM82 correlate with, and may be deduced from, the identity of the target genes which VGAM82 binds and inhibits, and the function of these target genes, as elaborated hereinbelow.

PTCRA (Accession NM_{—}138296) is a VGAM82 host target gene. PTCRA BINDING SITE is HOST TARGET binding site found in the 3′ untranslated region of mRNA encoded by PTCRA, corresponding to a HOST TARGET binding site such as BINDING SITE I, BINDING SITE II or BINDING SITE III. Table 2 illustrates the complementarity of the nucleotide sequences of PTCRA BINDING SITE, designated SEQ ID:2433, to the nucleotide sequence of VGAM82 RNA, herein designated VGAM RNA, also designated SEQ ID:417.

A function of VGAM82 is therefore inhibition of PTCRA (Accession NM_{—}138296). Accordingly, utilities of VGAM82 include diagnosis, prevention and treatment of diseases and clinical conditions associated with PTCRA. Putative Neuronal Cell Adhesion Molecule (PUNC, Accession XM_{—}116965) is another VGAM82 host target gene. PUNC BINDING SITE is HOST TARGET binding site found in the 3′ untranslated region of mRNA encoded by PUNC, corresponding to a HOST TARGET binding site such as BINDING SITE I, BINDING SITE II or BINDING SITE III. Table 2 illustrates the complementarity of the nucleotide sequences of PUNC BINDING SITE, designated SEQ ID:3469, to the nucleotide sequence of VGAM82 RNA, herein designated VGAM RNA, also designated SEQ ID:417.

Another function of VGAM82 is therefore inhibition of Putative Neuronal Cell Adhesion Molecule (PUNC, Accession XM_{—}116965), a gene which is implicated as a tumor suppressor gene. Accordingly, utilities of VGAM82 include diagnosis, prevention and treatment of diseases and clinical conditions associated with PUNC. The function of PUNC has been established by previous studies. Mouse Punc is a transmembrane protein that belongs to the immunoglobulin (Ig) super family. With a domain configuration of 4 Ig domains and 2 fibronectin type III repeats, Punc represents a novel subclass within the Ig super family. Sequence comparisons revealed that Punc is related to Ig super family members that are either associated with axons or function in axon guidance. Punc is highly expressed in the nervous system and limb buds of the developing mouse embryo. At midgestation, expression levels of Punc decrease sharply. By PCR using oligonucleotides based on the mouse Punc sequence, Salbaum (1999) isolated a partial human placenta PUNC cDNA (GenBank AF063936). Animal model experiments lend further support to the function of PUNC. Yang et al. (2001) generated mice deficient in Punc. These mice were not overtly ataxic and coordination improved with practice. However, the motor abilities of Punc-deficient mice were significantly decreased compared with wildtype littermates, similar to the deficits observed in vimentin (VIM; 193060)-deficient mice. No hearing deficits were detected. Yang et al. (2001) suggested that Punc expressed in Bergmann glia has a role in cerebellar control of motor coordination.

It is appreciated that the abovementioned animal model for PUNC is acknowledged by those skilled in the art as a scientifically valid animal model, as can be further appreciated from the publications sited hereinbelow.

Full details of the abovementioned studies are described in the following publications, the disclosure of which are hereby incorporated by reference:
Salbaum, J. M.: Genomic structure and chromosomal localization of the mouse gene Punc. Mammalian Genome 10: 107-111, 1999.; and
Yang, W.; Li, C.; Mansour, S. L.: Impaired motor coordination in mice that lack punc. Molec. Cell. Biol. 21: 6031-6043, 2001.

Further studies establishing the function and utilities of PUNC are found in John Hopkins OMIM database record ID 604184, and in sited publications numbered 220-221 listed in the bibliography section hereinbelow, which are also hereby incorporated by reference. KIAA0940 (Accession NM_{—}014912) is another VGAM82 host target gene. KIAA0940 BINDING SITE is HOST TARGET binding site found in the 3′ untranslated region of mRNA encoded by KIAA0940, corresponding to a HOST TARGET binding site such as BINDING SITE I, BINDING SITE II or BINDING SITE III. Table 2 illustrates the complementarity of the nucleotide sequences of KIAA0940 BINDING SITE, designated SEQ ID: 1586, to the nucleotide sequence of VGAM82 RNA, herein designated VGAM RNA, also designated SEQ ID:417.

Another function of VGAM82 is therefore inhibition of KIAA0940 (Accession NM_{—}014912). Accordingly, utilities of VGAM82 include diagnosis, prevention and treatment of diseases and clinical conditions associated with KIAA0940. Regulator of G-protein Signalling 18 (RGS18, Accession NM_{—}130782) is another VGAM82 host target gene. RGS18 BINDING SITE is HOST TARGET binding site found in the 3′ untranslated region of mRNA encoded by RGS18, corresponding to a HOST TARGET binding site such as BINDING SITE I, BINDING SITE II or BINDING SITE III. Table 2 illustrates the complementarity of the nucleotide sequences of RGS18 BINDING SITE, designated SEQ ID:2398, to the nucleotide sequence of VGAM82 RNA, herein designated VGAM RNA, also designated SEQ ID:417.

Another function of VGAM82 is therefore inhibition of Regulator of G-protein Signalling 18 (RGS18, Accession NM_{—}130782). Accordingly, utilities of VGAM82 include diagnosis, prevention and treatment of diseases and clinical conditions associated with RGS18. FIG. 1 further provides a conceptual description of a novel bioinformatically detected viral gene of the present invention, referred to here as Viral Genomic Address Messenger 83 (VGAM83) viral gene, which modulates expression of respective host target genes thereof, the function and utility of which host target genes is known in the art.

VGAM83 is a novel bioinformatically detected regulatory, non protein coding, viral micro RNA (miRNA) gene. The method by which VGAM83 was detected is described hereinabove with reference to FIGS. 1-8.

VGAM83 gene, herein designated VGAM GENE, is a viral gene contained in the genome of Vaccinia Virus. VGAM83 host target gene, herein designated VGAM HOST TARGET GENE, is a human gene contained in the human genome.

VGAM83 gene encodes a VGAM83 precursor RNA, herein designated VGAM PRECURSOR RNA. Similar to other miRNA genes, and unlike most ordinary genes, VGAM83 precursor RNA does not encode a protein. A nucleotide sequence identical or highly similar to the nucleotide sequence of VGAM83 precursor RNA is designated SEQ ID:69, and is provided hereinbelow with reference to the sequence listing part. Nucleotide sequence SEQ ID:69 is located at position 23600 relative to the genome of Vaccinia Virus.

VGAM83 precursor RNA folds onto itself, forming VGAM83 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM83 folded precursor RNA into VGAM83 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM83 RNA is designated SEQ ID:418, and is provided hereinbelow with reference to the sequence listing part.

VGAM83 host target gene, herein designated VGAM HOST TARGET GENE, encodes a corresponding messenger RNA, VGAM83 host target RNA, herein designated VGAM HOST TARGET RNA. VGAM83 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM83 RNA, herein designated VGAM RNA, binds complementarily to one or more host target binding sites located in untranslated regions of VGAM83 host target RNA, herein designated VGAM HOST TARGET RNA. This complementary binding is due to the fact that the nucleotide sequence of VGAM83 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM83 RNA, herein designated VGAM RNA, may have a different number of host target binding sites in untranslated regions of a VGAM83 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM83 RNA, herein designated VGAM RNA, to host target binding sites on VGAM83 host target RNA, herein designated VGAM HOST TARGET RNA, such as BINDING SITE I, BINDING SITE II and BINDING SITE III, inhibits translation of VGAM83 host target RNA into VGAM83 host target protein, herein designated VGAM HOST TARGET PROTEIN. VGAM host target protein is therefore outlined by a broken line.

It is appreciated that VGAM83 host target gene, herein designated VGAM HOST TARGET GENE, in fact represents a plurality of VGAM83 host target genes. The mRNA of each one of this plurality of VGAM83 host target genes comprises one or more host target binding sites, each having a nucleotide sequence which is at least partly complementary to VGAM83 RNA, herein designated VGAM RNA, and which when bound by VGAM83 RNA causes inhibition of translation of respective one or more VGAM83 host target proteins.

It is further appreciated by one skilled in the art that the mode of translational inhibition illustrated by FIG. 1 with specific reference to translational inhibition exerted by VGAM83 gene, herein designated VGAM GENE, on one or more VGAM83 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM83 is inhibition of expression of host target genes, as part of a novel viral mechanism of attacking a host. Accordingly, utilities of VGAM83 include diagnosis, prevention and treatment of viral infection by Vaccinia Virus. Specific functions, and accordingly utilities, of VGAM83 correlate with, and may be deduced from, the identity of the host target genes which VGAM83 binds and inhibits, and the function of these host target genes, as elaborated hereinbelow.

Nucleotide sequences of the VGAM83 precursor RNA, herein designated VGAM PRECURSOR RNA, and of the ‘diced’ VGAM83 RNA, herein designated VGAM RNA, and a schematic representation of the secondary folding of VGAM83 folded precursor RNA, herein designated VGAM FOLDED PRECURSOR RNA, of VGAM83 are further described hereinbelow with reference to Table 1.

Nucleotide sequences of host target binding sites, such as BINDING SITE-I, BINDING SITE-II and BINDING SITE-III of FIG. 1, found on VGAM83 host target RNA, and schematic representation of the complementarity of each of these host target binding sites to VGAM83 RNA, herein designated VGAM RNA, are described hereinbelow with reference to Table 2.

As mentioned hereinabove with reference to FIG. 1, a function of VGAM83 gene, herein designated VGAM is inhibition of expression of VGAM83 target genes. It is appreciated that specific functions, and accordingly utilities, of VGAM83 correlate with, and may be deduced from, the identity of the target genes which VGAM83 binds and inhibits, and the function of these target genes, as elaborated hereinbelow.

ATPase, H+/K+ Transporting, Nongastric, Alpha Polypeptide (ATP12A, Accession NM_{—}001676) is a VGAM83 host target gene. ATP12A BINDING SITE is HOST TARGET binding site found in the 3′ untranslated region of mRNA encoded by ATP12A, corresponding to a HOST TARGET binding site such as BINDING SITE I, BINDING SITE II or BINDING SITE III. Table 2 illustrates the complementarity of the nucleotide sequences of ATP12A BINDING SITE, designated SEQ ID:846, to the nucleotide sequence of VGAM83 RNA, herein designated VGAM RNA, also designated SEQ ID:418.

A function of VGAM83 is therefore inhibition of ATPase, H+/K+ Transporting, Nongastric, Alpha Polypeptide (ATP12A, Accession NM_{—}001676). Accordingly, utilities of VGAM83 include diagnosis, prevention and treatment of diseases and clinical conditions associated with ATP12A. Crystallin, Gamma S(CRYGS, Accession NM_{—}017541) is another VGAM83 host target gene. CRYGS BINDING SITE is HOST TARGET binding site found in the 3′ untranslated region of mRNA encoded by CRYGS, corresponding to a HOST TARGET binding site such as BINDING SITE I, BINDING SITE II or BINDING SITE III. Table 2 illustrates the complementarity of the nucleotide sequences of CRYGS BINDING SITE, designated SEQ ID:1722, to the nucleotide sequence of VGAM83 RNA, herein designated VGAM RNA, also designated SEQ ID:418.

Another function of VGAM83 is therefore inhibition of Crystallin, Gamma S(CRYGS, Accession NM_{—}017541), a gene which is a dominant structural components of the vertebrate eye lens. Accordingly, utilities of VGAM83 include diagnosis, prevention and treatment of diseases and clinical conditions associated with CRYGS. The function of CRYGS has been established by previous studies. The beta-crystallins and gamma-crystallins of the mammalian lens form a super family of related proteins which are apparently derived from a common ancestral gene; see CRYGA (OMIM Ref. No. 123660). An exceptional member of this super family is gamma-S (formerly beta-S). In contrast to the beta-crystallins which associate in various combinations to form low or high molecular weight aggregates, gamma-S is, like the other gamma-crystallins, a monomeric protein. It was suggested by den Dunnen et al. (1985) that all members of the human gamma-crystallin gene family are located on chromosome 2. However, study of hamster-human somatic cell hybrids with a bovine cDNA probe for CRYGS led Wijnen et al. (1989) to the conclusion that this gene is located on human chromosome 3.

Full details of the abovementioned studies are described in the following publications, the disclosure of which are hereby incorporated by reference:
den Dunnen, J. T.; Jongbloed, R. J. E.; Geurts van Kessel, A. H. M.; Schoenmakers, J. G. G.: Human lens gammacrystallin sequences are located in the p12-qter region of chromosome 2. Hum. Genet. 70: 217-221, 1985.; and
Wijnen, J. T.; Oldenburg, M.; Bloemendal, H.; Meera Khan, P.: GS(gamma-S)-crystallin (CRYGS) assignment to chromosome 3. (Abstract) Cytogenet. Cell Genet. 51: 1108 only, 1989.

Further studies establishing the function and utilities of CRYGS are found in John Hopkins OMIM database record ID 123730, and in sited publications numbered 2809 listed in the bibliography section hereinbelow, which are also hereby incorporated by reference. Lipin 2 (LPIN2, Accession NM_{—}014646) is another VGAM83 host target gene. LPIN2 BINDING SITE is HOST TARGET binding site found in the 3′ untranslated region of mRNA encoded by LPIN2, corresponding to a HOST TARGET binding site such as BINDING SITE I, BINDING SITE II or BINDING SITE III. Table 2 illustrates the complementarity of the nucleotide sequences of LPIN2 BINDING SITE, designated SEQ ID:1514, to the nucleotide sequence of VGAM83 RNA, herein designated VGAM RNA, also designated SEQ ID:418.

Another function of VGAM83 is therefore inhibition of Lipin 2 (LPIN2, Accession NM_{—}014646). Accordingly, utilities of VGAM83 include diagnosis, prevention and treatment of diseases and clinical conditions associated with LPIN2. KIAA1786 (Accession XM_{—}038436) is another VGAM83 host target gene. KIAA1786 BINDING SITE is HOST TARGET binding site found in the 3′ untranslated region of mRNA encoded by KIAA1786, corresponding to a HOST TARGET binding site such as BINDING SITE I, BINDING SITE II or BINDING SITE III. Table 2 illustrates the complementarity of the nucleotide sequences of KIAA1786 BINDING SITE, designated SEQ ID:2738, to the nucleotide sequence of VGAM83 RNA, herein designated VGAM RNA, also designated SEQ ID:418.

Another function of VGAM83 is therefore inhibition of KIAA1786 (Accession XM_{—}038436). Accordingly, utilities of VGAM83 include diagnosis, prevention and treatment of diseases and clinical conditions associated with KIAA1786. LOC222008 (Accession XM_{—}168361) is another VGAM83 host target gene. LOC222008 BINDING SITE is HOST TARGET binding site found in the 3′ untranslated region of mRNA encoded by LOC222008, corresponding to a HOST TARGET binding site such as BINDING SITE I, BINDING SITE II or BINDING SITE III. Table 2 illustrates the complementarity of the nucleotide sequences of LOC222008 BINDING SITE, designated SEQ ID:3629, to the nucleotide sequence of VGAM83 RNA, herein designated VGAM RNA, also designated SEQ ID:418.

Another function of VGAM83 is therefore inhibition of LOC222008 (Accession XM_{—}168361). Accordingly, utilities of VGAM83 include diagnosis, prevention and treatment of diseases and clinical conditions associated with LOC222008. LOC90639 (Accession XM_{—}033092) is another VGAM83 host target gene. LOC90639 BINDING SITE is HOST TARGET binding site found in the 5′ untranslated region of mRNA encoded by LOC90639, corresponding to a HOST TARGET binding site such as BINDING SITE I, BINDING SITE II or BINDING SITE III. Table 2 illustrates the complementarity of the nucleotide sequences of LOC90639 BINDING SITE, designated SEQ ID:2663, to the nucleotide sequence of VGAM83 RNA, herein designated VGAM RNA, also designated SEQ ID:418.

Another function of VGAM83 is therefore inhibition of LOC90639 (Accession XM_{—}033092). Accordingly, utilities of VGAM83 include diagnosis, prevention and treatment of diseases and clinical conditions associated with LOC90639. FIG. 1 further provides a conceptual description of a novel bioinformatically detected viral gene of the present invention, referred to here as Viral Genomic Address Messenger 84 (VGAM84) viral gene, which modulates expression of respective host target genes thereof, the function and utility of which host target genes is known in the art.

VGAM84 is a novel bioinformatically detected regulatory, non protein coding, viral micro RNA (miRNA) gene. The method by which VGAM84 was detected is described hereinabove with reference to FIGS. 1-8.

VGAM84 gene, herein designated VGAM GENE, is a viral gene contained in the genome of Vaccinia Virus. VGAM84 host target gene, herein designated VGAM HOST TARGET GENE, is a human gene contained in the human genome.

VGAM84 gene encodes a VGAM84 precursor RNA, herein designated VGAM PRECURSOR RNA. Similar to other miRNA genes, and unlike most ordinary genes, VGAM84 precursor RNA does not encode a protein. A nucleotide sequence identical or highly similar to the nucleotide sequence of VGAM84 precursor RNA is designated SEQ ID:70, and is provided hereinbelow with reference to the sequence listing part. Nucleotide sequence SEQ ID:70 is located at position 27761 relative to the genome of Vaccinia Virus.

VGAM84 precursor RNA folds onto itself, forming VGAM84 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM84 folded precursor RNA into VGAM84 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM84 RNA is designated SEQ ID:419, and is provided hereinbelow with reference to the sequence listing part.

VGAM84 host target gene, herein designated VGAM HOST TARGET GENE, encodes a corresponding messenger RNA, VGAM84 host target RNA, herein designated VGAM HOST TARGET RNA. VGAM84 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM84 RNA, herein designated VGAM RNA, binds complementarily to one or more host target binding sites located in untranslated regions of VGAM84 host target RNA, herein designated VGAM HOST TARGET RNA. This complementary binding is due to the fact that the nucleotide sequence of VGAM84 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM84 RNA, herein designated VGAM RNA, may have a different number of host target binding sites in untranslated regions of a VGAM84 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM84 RNA, herein designated VGAM RNA, to host target binding sites on VGAM84 host target RNA, herein designated VGAM HOST TARGET RNA, such as BINDING SITE I, BINDING SITE II and BINDING SITE III, inhibits translation of VGAM84 host target RNA into VGAM84 host target protein, herein designated VGAM HOST TARGET PROTEIN. VGAM host target protein is therefore outlined by a broken line.

It is appreciated that VGAM84 host target gene, herein designated VGAM HOST TARGET GENE, in fact represents a plurality of VGAM84 host target genes. The mRNA of each one of this plurality of VGAM84 host target genes comprises one or more host target binding sites, each having a nucleotide sequence which is at least partly complementary to VGAM84 RNA, herein designated VGAM RNA, and which when bound by VGAM84 RNA causes inhibition of translation of respective one or more VGAM84 host target proteins.

It is further appreciated by one skilled in the art that the mode of translational inhibition illustrated by FIG. 1 with specific reference to translational inhibition exerted by VGAM84 gene, herein designated VGAM GENE, on one or more VGAM84 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM84 is inhibition of expression of host target genes, as part of a novel viral mechanism of attacking a host. Accordingly, utilities of VGAM84 include diagnosis, prevention and treatment of viral infection by Vaccinia Virus. Specific functions, and accordingly utilities, of VGAM84 correlate with, and may be deduced from, the identity of the host target genes which VGAM84 binds and inhibits, and the function of these host target genes, as elaborated hereinbelow.

Nucleotide sequences of the VGAM84 precursor RNA, herein designated VGAM PRECURSOR RNA, and of the ‘diced’ VGAM84 RNA, herein designated VGAM RNA, and a schematic representation of the secondary folding of VGAM84 folded precursor RNA, herein designated VGAM FOLDED PRECURSOR RNA, of VGAM84 are further described hereinbelow with reference to Table 1.

Nucleotide sequences of host target binding sites, such as BINDING SITE-I, BINDING SITE-II and BINDING SITE-III of FIG. 1, found on VGAM84 host target RNA, and schematic representation of the complementarity of each of these host target binding sites to VGAM84 RNA, herein designated VGAM RNA, are described hereinbelow with reference to Table 2.

As mentioned hereinabove with reference to FIG. 1, a function of VGAM84 gene, herein designated VGAM is inhibition of expression of VGAM84 target genes. It is appreciated that specific functions, and accordingly utilities, of VGAM84 correlate with, and may be deduced from, the identity of the target genes which VGAM84 binds and inhibits, and the function of these target genes, as elaborated hereinbelow.

Adaptor-related Protein Complex 1, Beta 1 Subunit (AP1B1, Accession NM_{—}001127) is a VGAM84 host target gene. AP1B1 BINDING SITE is HOST TARGET binding site found in the 3′ untranslated region of mRNA encoded by AP1B1, corresponding to a HOST TARGET binding site such as BINDING SITE I, BINDING SITE II or BINDING SITE III. Table 2 illustrates the complementarity of the nucleotide sequences of AP1B1 BINDING SITE, designated SEQ ID:803, to the nucleotide sequence of VGAM84 RNA, herein designated VGAM RNA, also designated SEQ ID:419.

A function of VGAM84 is therefore inhibition of Adaptor-related Protein Complex 1, Beta 1 Subunit (AP1B1, Accession NM_{—}001127), a gene which plays a role in protein sorting in the late-golgi/trans-golgi network (tgn) and/or endosomes. Accordingly, utilities of VGAM84 include diagnosis, prevention and treatment of diseases and clinical conditions associated with AP1B1. The function of AP1B1 has been established by previous studies. A 140-kb homozygous deletion in 22q12 in a sporadic meningioma directed Peyrard et al. (1994) to the cloning and characterization of a new member of the human beta-adaptin gene family, which was named BAM22 for ‘beta-adaptin-meningioma-chromosome 22.’ The BAM22 gene was totally inactivated in the tumor with homozygous deletion. Northern blot analysis of 70 sporadic meningiomas showed specific loss of expression in 8 tumors, suggesting inactivation of BAM22. Based on this, Peyrard et al. (1994) suggested that BAM22 is a second chromosome 22 locus important in meningioma development and second in importance to the neurofibromatosis type 2 gene (NF2; 101000). The likelihood that multiple loci on chromosome 22 are involved in the oncogenesis of meningioma is suggested by the facts that monosomy 22 is observed in as many as 65% of tumors (Zankl and Zang, 1980); some meningiomas have chromosome 22 deletions not encompassing the NF2 gene region and do not show mutations in the NF2 gene; and constitutional ring chromosome 22 has been observed in young patients with multiple tumors (Arinami et al., 1986; Petrella et al., 1993).

Full details of the abovementioned studies are described in the following publications, the disclosure of which are hereby incorporated by reference:
Peyrard, M.; Pan, H.-Q.; Kedra, D.; Fransson, I.; Swahn, S.; Hartman, K.; Clifton, S. W.; Roe, B. A.; Dumanski, J. P.: Structure of the promoter and genomic organization of the human beta-prime-adaptin gene (BAM22) from chromosome 22q12. Genomics 36: 112-117, 1996.; and
Zankl, H.; Zang, K. D.: Correlations between clinical and cytogenetical data in 180 human meningiomas. Cancer Genet. Cytogenet. 1: 351-356, 1980.

Further studies establishing the function and utilities of AP1B1 are found in John Hopkins OMIM database record ID 600157, and in sited publications numbered 170 and 3034-1711 listed in the bibliography section hereinbelow, which are also hereby incorporated by reference. IQ Motif Containing GTPase Activating Protein 1 (IQGAP1, Accession XM_{—}044539) is another VGAM84 host target gene. IQGAP1 BINDING SITE is HOST TARGET binding site found in the 3′ untranslated region of mRNA encoded by IQGAP1, corresponding to a HOST TARGET binding site such as BINDING SITE I, BINDING SITE II or BINDING SITE III. Table 2 illustrates the complementarity of the nucleotide sequences of IQGAP1 BINDING SITE, designated SEQ ID:2840, to the nucleotide sequence of VGAM84 RNA, herein designated VGAM RNA, also designated SEQ ID:419.

Another function of VGAM84 is therefore inhibition of IQ Motif Containing GTPase Activating Protein 1 (IQGAP1, Accession XM_{—}044539), a gene which inhibits GTPase activity of ras family of GTP binding proteins Cdc42Hs and rac. Accordingly, utilities of VGAM84 include diagnosis, prevention and treatment of diseases and clinical conditions associated with IQGAP1. The function of IQGAP1 has been established by previous studies. Sugimoto et al. (2001) demonstrated that IQGAP1, a negative regulator of cell-cell adhesion, is up-regulated by gene amplification at 15q26 in 2 gastric cancer cell lines. Amplification at 15q26 had been found in various malignancies, including breast cancers, and FES (OMIM Ref. No. 190030) and/or IGF1R (OMIM Ref. No. 147370) had been identified as targets for gene amplification in breast cancer, melanoma, and pancreatic adenocarcinoma. In contrast, Sugimoto et al. (2001) found that both genes are located telomeric to the amplicon at 15q26 in the 2 gastric cancer cell lines they studied. Fukata et al. (2002) found that IQGAP1, an effector of RAC1 (OMIM Ref. No. 602048) and CDC42, interacts with CLIP170 (RSN; 179838). In Vero fibroblasts, IQGAP1 localized at the polarized leading edge. Expression of a C-terminal fragment of IQGAP1 that included the CLIP170-binding region delocalized GFP-CLIP170 from the tips of microtubules and altered the microtubule array. The authors found that activated RAC1/CDC42, IQGAP1, and CLIP170 form a tripartite complex. Furthermore, expression of an IQGAP1 mutant defective in RAC1/CDC42 binding induced multiple leading edges. These results indicated that RAC1/CDC42 marks special cortical spots where the IQGAP1 and CLIP170 complex is targeted, leading to a polarized microtubule array and cell polarization

Full details of the abovementioned studies are described in the following publications, the disclosure of which are hereby incorporated by reference:
Fukata, M.; Watanabe, T.; Noritake, J.; Nakagawa, M.; Yamaga, M.; Kuroda, S.; Matsuura, Y.; Iwamatsu, A.; Perez, F.; Kaibuchi, K.: Rac1 and Cdc42 capture microtubules through IQGAP1 and CLIP-170. Cell 109: 873-885, 2002.; and
Hart, M. J.; Callow, M. G.; Souza, B.; Polakis, P.: IQGAP1, a calmodulin-binding protein with a rasGAP-related domain, is a potential effector for cdc42Hs. EMBO J. 15: 2997-3005, 1996.

Further studies establishing the function and utilities of IQGAP1 are found in John Hopkins OMIM database record ID 603379, and in sited publications numbered 38 and 1689-1691 listed in the bibliography section hereinbelow, which are also hereby incorporated by reference. Phosphodiesterase 6A, CGMP-specific, Rod, Alpha (PDE6A, Accession NM_{—}000440) is another VGAM84 host target gene. PDE6A BINDING SITE is HOST TARGET binding site found in the 3′ untranslated region of mRNA encoded by PDE6A, corresponding to a HOST TARGET binding site such as BINDING SITE I, BINDING SITE II or BINDING SITE III. Table 2 illustrates the complementarity of the nucleotide sequences of PDE6A BINDING SITE, designated SEQ ID:744, to the nucleotide sequence of VGAM84 RNA, herein designated VGAM RNA, also designated SEQ ID:419.

Another function of VGAM84 is therefore inhibition of Phosphodiesterase 6A, CGMP-specific, Rod, Alpha (PDE6A, Accession NM_{—}000440). Accordingly, utilities of VGAM84 include diagnosis, prevention and treatment of diseases and clinical conditions associated with PDE6A. Phosphatase and Tensin Homolog (mutated in multiple advanced cancers 1) (PTEN, Accession NM_{—}000314) is another VGAM84 host target gene. PTEN BINDING SITE is HOST TARGET binding site found in the 3′ untranslated region of mRNA encoded by PTEN, corresponding to a HOST TARGET binding site such as BINDING SITE I, BINDING SITE II or BINDING SITE III. Table 2 illustrates the complementarity of the nucleotide sequences of PTEN BINDING SITE, designated SEQ ID:728, to the nucleotide sequence of VGAM84 RNA, herein designated VGAM RNA, also designated SEQ ID:419.

Another function of VGAM84 is therefore inhibition of Phosphatase and Tensin Homolog (mutated in multiple advanced cancers 1) (PTEN, Accession NM_{—}000314). Accordingly, utilities of VGAM84 include diagnosis, prevention and treatment of diseases and clinical conditions associated with PTEN. SH3-domain GRB2-like 2 (SH3GL2, Accession NM_{—}003026) is another VGAM84 host target gene. SH3GL2 BINDING SITE is HOST TARGET binding site found in the 3′ untranslated region of mRNA encoded by SH3GL2, corresponding to a HOST TARGET binding site such as BINDING SITE I, BINDING SITE II or BINDING SITE III. Table 2 illustrates the complementarity of the nucleotide sequences of SH3GL2 BINDING SITE, designated SEQ ID:985, to the nucleotide sequence of VGAM84 RNA, herein designated VGAM RNA, also designated SEQ ID:419.

Another function of VGAM84 is therefore inhibition of SH3-domain GRB2-like 2 (SH3GL2, Accession NM_{—}003026), a gene which plays a role in synaptic vesicle recycling, in particular in clathrin-mediated vesicle endocytosis. Accordingly, utilities of VGAM84 include diagnosis, prevention and treatment of diseases and clinical conditions associated with SH3GL2. The function of SH3GL2 has been established by previous studies. Endophilin-1 is a presynaptic protein that binds to dynamin (see OMIM Ref. No. 602377), a GTPase that is implicated in endocytosis and recycling of synaptic vesicles. Schmidt et al. (1999) showed that endophilin-1 is essential for the formation of synaptic-like microvesicles from the plasma membrane. Endophilin-1 exhibits lysophosphatidic acid acyltransferase (LPAAT) activity. Endophilin-1-mediated synaptic-like microvesicle formation requires the transfer of the unsaturated fatty acid arachidonate to lysophosphatidic acid, converting it to phosphatidic acid. A deletion mutant lacking the SH3 domain, through which endophilin-1 interacts with dynamin, still exhibits LPAAT activity but no longer mediates synaptic-like microvesicle formation. Schmidt et al. (1999) concluded that endophilin-1 may induce negative membrane curvature by converting an inverted cone-shaped lipid to a cone-shaped lipid in the cytoplasmic leaflet of the bilayer. Schmidt et al. (1999) proposed that through this action, endophilin-1 works with dynamin to mediate synaptic vesicle invagination from the plasma membrane and fission. By screening with C-terminal CIN85 (OMIM Ref. No. 300374) as bait, Soubeyran et al. (2002) identified endophilins A1, A2 (OMIM Ref. No. 601768), and A3 (OMIM Ref. No. 603362) as constitutive interactors, via their SH3 domains, with proline-rich sequences of CIN85. EGF (OMIM Ref. No. 131530) stimulation results in a complex of CBL, CIN85, endophilins, and EGF receptors. Confocal microscopy demonstrated a diffuse cytoplasmic distribution in resting cells and a colocalization with EGF receptors in endocytic vesicles after EGF stimulation. Analysis with dominant interfering forms of CIN85 suggested that CIN85 is dispensable for polyubiquitination of EGF receptors but that it may be critical for the regulation of receptor endocytosis and lysosomal degradation. Soubeyran et al. (2002) concluded that CIN85 has a role in the control of postmembrane events such as targeting receptors for degradation and regulation of gene transcription, possibly by binding to multiple adaptor proteins.

Full details of the abovementioned studies are described in the following publications, the disclosure of which are hereby incorporated by reference:
Schmidt, A.; Wolde, M.; Thiele, C.; Fest, W.; Kratzin, H.; Podtelejnikov, A. V.; Witke, W.; Huttner, W. B.; Soling, H.-D.: Endophilin I mediates synaptic vesicle formation by transfer of arachidonate to lysophosphatidic acid. Nature 401: 133-141, 1999.; and
Soubeyran, P.; Kowanetz, K.; Szymkiewicz, I.; Langdon, W. Y.; Dikic, I.: Cbl-CIN85-endophilin complex mediates ligand-induced downregulation of EGF receptors. Nature 416: 183-187, 2002.

Further studies establishing the function and utilities of SH3GL2 are found in John Hopkins OMIM database record ID 604465, and in sited publications numbered 2029, 111 and 2510 listed in the bibliography section hereinbelow, which are also hereby incorporated by reference. Chromosome 1 Open Reading Frame 16 (Clorf16, Accession NM_{—}014837) is another VGAM84 host target gene. Clorf16 BINDING SITE is HOST TARGET binding site found in the 3′ untranslated region of mRNA encoded by Clorf16, corresponding to a HOST TARGET binding site such as BINDING SITE I, BINDING SITE II or BINDING SITE III. Table 2 illustrates the complementarity of the nucleotide sequences of Clorf16 BINDING SITE, designated SEQ ID:1567, to the nucleotide sequence of VGAM84 RNA, herein designated VGAM RNA, also designated SEQ ID:419.

Another function of VGAM84 is therefore inhibition of Chromosome 1 Open Reading Frame 16 (Clorf16, Accession NM_{—}014837). Accordingly, utilities of VGAM84 include diagnosis, prevention and treatment of diseases and clinical conditions associated with Clorf16. DKFZp547A023 (Accession XM_{—}052065) is another VGAM84 host target gene. DKFZp547A023 BINDING SITE is HOST TARGET binding site found in the 3′ untranslated region of mRNA encoded by DKFZp547A023, corresponding to a HOST TARGET binding site such as BINDING SITE I, BINDING SITE II or BINDING SITE III. Table 2 illustrates the complementarity of the nucleotide sequences of DKFZp547A023 BINDING SITE, designated SEQ ID:2956, to the nucleotide sequence of VGAM84 RNA, herein designated VGAM RNA, also designated SEQ ID:419.

Another function of VGAM84 is therefore inhibition of DKFZp547A023 (Accession XM_{—}052065). Accordingly, utilities of VGAM84 include diagnosis, prevention and treatment of diseases and clinical conditions associated with DKFZp547A023. FLJ11850 (Accession NM_{—}022741) is another VGAM84 host target gene. FLJ11850 BINDING SITE is HOST TARGET binding site found in the 5′ untranslated region of mRNA encoded by FLJ11850, corresponding to a HOST TARGET binding site such as BINDING SITE I, BINDING SITE II or BINDING SITE III. Table 2 illustrates the complementarity of the nucleotide sequences of FLJ11850 BINDING SITE, designated SEQ ID:2001, to the nucleotide sequence of VGAM84 RNA, herein designated VGAM RNA, also designated SEQ ID:419.

Another function of VGAM84 is therefore inhibition of FLJ11850 (Accession NM_{—}022741). Accordingly, utilities of VGAM84 include diagnosis, prevention and treatment of diseases and clinical conditions associated with FLJ11850. FLJ12888 (Accession NM_{—}024945) is another VGAM84 host target gene. FLJ12888 BINDING SITE is HOST TARGET binding site found in the 3′ untranslated region of mRNA encoded by FLJ12888, corresponding to a HOST TARGET binding site such as BINDING SITE I, BINDING SITE II or BINDING SITE III. Table 2 illustrates the complementarity of the nucleotide sequences of FLJ12888 BINDING SITE, designated SEQ ID:2115, to the nucleotide sequence of VGAM84 RNA, herein designated VGAM RNA, also designated SEQ ID:419.

Another function of VGAM84 is therefore inhibition of FLJ12888 (Accession NM_{—}024945). Accordingly, utilities of VGAM84 include diagnosis, prevention and treatment of diseases and clinical conditions associated with FLJ12888. FLJ21596 (Accession NM_{—}024823) is another VGAM84 host target gene. FLJ21596 BINDING SITE is HOST TARGET binding site found in the 3′ untranslated region of mRNA encoded by FLJ21596, corresponding to a HOST TARGET binding site such as BINDING SITE I, BINDING SITE II or BINDING SITE III. Table 2 illustrates the complementarity of the nucleotide sequences of FLJ21596 BINDING SITE, designated SEQ ID:2092, to the nucleotide sequence of VGAM84 RNA, herein designated VGAM RNA, also designated SEQ ID:419.

Another function of VGAM84 is therefore inhibition of FLJ21596 (Accession NM_{—}024823). Accordingly, utilities of VGAM84 include diagnosis, prevention and treatment of diseases and clinical conditions associated with FLJ21596. KIAA1878 (Accession XM_{—}166256) is another VGAM84 host target gene. KIAA1878 BINDING SITE is HOST TARGET binding site found in the 3′ untranslated region of mRNA encoded by KIAA1878, corresponding to a HOST TARGET binding site such as BINDING SITE I, BINDING SITE II or BINDING SITE III. Table 2 illustrates the complementarity of the nucleotide sequences of KIAA1878 BINDING SITE, designated SEQ ID:3539, to the nucleotide sequence of VGAM84 RNA, herein designated VGAM RNA, also designated SEQ ID:419.

Another function of VGAM84 is therefore inhibition of KIAA1878 (Accession XM_{—}166256). Accordingly, utilities of VGAM84 include diagnosis, prevention and treatment of diseases and clinical conditions associated with KIAA1878. Neurexophilin 3 (NXPH3, Accession XM_{—}037847) is another VGAM84 host target gene. NXPH3 BINDING SITE is HOST TARGET binding site found in the 3′ untranslated region of mRNA encoded by NXPH3, corresponding to a HOST TARGET binding site such as BINDING SITE I, BINDING SITE II or BINDING SITE III. Table 2 illustrates the complementarity of the nucleotide sequences of NXPH3 BINDING SITE, designated SEQ ID:2728, to the nucleotide sequence of VGAM84 RNA, herein designated VGAM RNA, also designated SEQ ID:419.

Another function of VGAM84 is therefore inhibition of Neurexophilin 3 (NXPH3, Accession XM_{—}037847). Accordingly, utilities of VGAM84 include diagnosis, prevention and treatment of diseases and clinical conditions associated with NXPH3. Synovial Sarcoma Translocation Gene On Chromosome 18-like 1 (SS18Li, Accession XM_{—}037202) is another VGAM84 host target gene. SS18L1 BINDING SITE is HOST TARGET binding site found in the 3′ untranslated region of mRNA encoded by SS18Li, corresponding to a HOST TARGET binding site such as BINDING SITE I, BINDING SITE II or BINDING SITE III. Table 2 illustrates the complementarity of the nucleotide sequences of SS18L1 BINDING SITE, designated SEQ ID:2719, to the nucleotide sequence of VGAM84 RNA, herein designated VGAM RNA, also designated SEQ ID:419.

Another function of VGAM84 is therefore inhibition of Synovial Sarcoma Translocation Gene On Chromosome 18-like 1 (SS18Li, Accession XM_{—}037202). Accordingly, utilities of VGAM84 include diagnosis, prevention and treatment of diseases and clinical conditions associated with SS18L1. Synaptotagmin XIII (SYT13, Accession XM_{—}167880) is another VGAM84 host target gene. SYT13 BINDING SITE is HOST TARGET binding site found in the 3′ untranslated region of mRNA encoded by SYT13, corresponding to a HOST TARGET binding site such as BINDING SITE I, BINDING SITE II or BINDING SITE III. Table 2 illustrates the complementarity of the nucleotide sequences of SYT13 BINDING SITE, designated SEQ ID:3605, to the nucleotide sequence of VGAM84 RNA, herein designated VGAM RNA, also designated SEQ ID:419.

Another function of VGAM84 is therefore inhibition of Synaptotagmin XIII (SYT13, Accession XM_{—}167880). Accordingly, utilities of VGAM84 include diagnosis, prevention and treatment of diseases and clinical conditions associated with SYT13. LOC134301 (Accession XM_{—}059705) is another VGAM84 host target gene. LOC134301 BINDING SITE is HOST TARGET binding site found in the 3′ untranslated region of mRNA encoded by LOC134301, corresponding to a HOST TARGET binding site such as BINDING SITE I, BINDING SITE II or BINDING SITE III. Table 2 illustrates the complementarity of the nucleotide sequences of LOC134301 BINDING SITE, designated SEQ ID:3016, to the nucleotide sequence of VGAM84 RNA, herein designated VGAM RNA, also designated SEQ ID:419.

Another function of VGAM84 is therefore inhibition of LOC134301 (Accession XM_{—}059705). Accordingly, utilities of VGAM84 include diagnosis, prevention and treatment of diseases and clinical conditions associated with LOC134301. LOC151438 (Accession XM_{—}098060) is another VGAM84 host target gene. LOC151438 BINDING SITE is HOST TARGET binding site found in the 3′ untranslated region of mRNA encoded by LOC151438, corresponding to a HOST TARGET binding site such as BINDING SITE I, BINDING SITE II or BINDING SITE III. Table 2 illustrates the complementarity of the nucleotide sequences of LOC151438 BINDING SITE, designated SEQ ID:3346, to the nucleotide sequence of VGAM84 RNA, herein designated VGAM RNA, also designated SEQ ID:419.

Another function of VGAM84 is therefore inhibition of LOC151438 (Accession XM_{—}098060). Accordingly, utilities of VGAM84 include diagnosis, prevention and treatment of diseases and clinical conditions associated with LOC151438. LOC158696 (Accession XM_{—}088644) is another VGAM84 host target gene. LOC158696 BINDING SITE is HOST TARGET binding site found in the 3′ untranslated region of mRNA encoded by LOC158696, corresponding to a HOST TARGET binding site such as BINDING SITE I, BINDING SITE II or BINDING SITE III. Table 2 illustrates the complementarity of the nucleotide sequences of LOC158696 BINDING SITE, designated SEQ ID:3223, to the nucleotide sequence of VGAM84 RNA, herein designated VGAM RNA, also designated SEQ ID:419.

Another function of VGAM84 is therefore inhibition of LOC158696 (Accession XM_{—}088644). Accordingly, utilities of VGAM84 include diagnosis, prevention and treatment of diseases and clinical conditions associated with LOC158696. LOC221975 (Accession XM_{—}166534) is another VGAM84 host target gene. LOC221975 BINDING SITE is HOST TARGET binding site found in the 3′ untranslated region of mRNA encoded by LOC221975, corresponding to a HOST TARGET binding site such as BINDING SITE I, BINDING SITE II or BINDING SITE III. Table 2 illustrates the complementarity of the nucleotide sequences of LOC221975 BINDING SITE, designated SEQ ID:3570, to the nucleotide sequence of VGAM84 RNA, herein designated VGAM RNA, also designated SEQ ID:419.

Another function of VGAM84 is therefore inhibition of LOC221975 (Accession XM_{—}166534). Accordingly, utilities of VGAM84 include diagnosis, prevention and treatment of diseases and clinical conditions associated with LOC221975. LOC84549 (Accession NM_{—}032509) is another VGAM84 host target gene. LOC84549 BINDING SITE is HOST TARGET binding site found in the 3′ untranslated region of mRNA encoded by LOC84549, corresponding to a HOST TARGET binding site such as BINDING SITE I, BINDING SITE II or BINDING SITE III. Table 2 illustrates the complementarity of the nucleotide sequences of LOC84549 BINDING SITE, designated SEQ ID:2256, to the nucleotide sequence of VGAM84 RNA, herein designated VGAM RNA, also designated SEQ ID:419.

Another function of VGAM84 is therefore inhibition of LOC84549 (Accession NM_{—}032509). Accordingly, utilities of VGAM84 include diagnosis, prevention and treatment of diseases and clinical conditions associated with LOC84549. LOC90459 (Accession XM_{—}031826) is another VGAM84 host target gene. LOC90459 BINDING SITE is HOST TARGET binding site found in the 3′ untranslated region of mRNA encoded by LOC90459, corresponding to a HOST TARGET binding site such as BINDING SITE I, BINDING SITE II or BINDING SITE III. Table 2 illustrates the complementarity of the nucleotide sequences of LOC90459 BINDING SITE, designated SEQ ID:2644, to the nucleotide sequence of VGAM84 RNA, herein designated VGAM RNA, also designated SEQ ID:419.

Another function of VGAM84 is therefore inhibition of LOC90459 (Accession XM_{—}031826). Accordingly, utilities of VGAM84 include diagnosis, prevention and treatment of diseases and clinical conditions associated with LOC90459. LOC90529 (Accession XM_{—}032350) is another VGAM84 host target gene. LOC90529 BINDING SITE is HOST TARGET binding site found in the 3′ untranslated region of mRNA encoded by LOC90529, corresponding to a HOST TARGET binding site such as BINDING SITE I, BINDING SITE II or BINDING SITE III. Table 2 illustrates the complementarity of the nucleotide sequences of LOC90529 BINDING SITE, designated SEQ ID:2652, to the nucleotide sequence of VGAM84 RNA, herein designated VGAM RNA, also designated SEQ ID:419.

Another function of VGAM84 is therefore inhibition of LOC90529 (Accession XM_{—}032350). Accordingly, utilities of VGAM84 include diagnosis, prevention and treatment of diseases and clinical conditions associated with LOC90529. FIG. 1 further provides a conceptual description of a novel bioinformatically detected viral gene of the present invention, referred to here as Viral Genomic Address Messenger 85 (VGAM85) viral gene, which modulates expression of respective host target genes thereof, the function and utility of which host target genes is known in the art.

VGAM85 is a novel bioinformatically detected regulatory, non protein coding, viral micro RNA (miRNA) gene. The method by which VGAM85 was detected is described hereinabove with reference to FIGS. 1-8.

VGAM85 gene, herein designated VGAM GENE, is a viral gene contained in the genome of Vaccinia Virus. VGAM85 host target gene, herein designated VGAM HOST TARGET GENE, is a human gene contained in the human genome.

VGAM85 gene encodes a VGAM85 precursor RNA, herein designated VGAM PRECURSOR RNA. Similar to other miRNA genes, and unlike most ordinary genes, VGAM85 precursor RNA does not encode a protein. A nucleotide sequence identical or highly similar to the nucleotide sequence of VGAM85 precursor RNA is designated SEQ ID:71, and is provided hereinbelow with reference to the sequence listing part. Nucleotide sequence SEQ ID:71 is located at position 26098 relative to the genome of Vaccinia Virus.

VGAM85 precursor RNA folds onto itself, forming VGAM85 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM85 folded precursor RNA into VGAM85 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM85 RNA is designated SEQ ID:420, and is provided hereinbelow with reference to the sequence listing part.

VGAM85 host target gene, herein designated VGAM HOST TARGET GENE, encodes a corresponding messenger RNA, VGAM85 host target RNA, herein designated VGAM HOST TARGET RNA. VGAM85 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM85 RNA, herein designated VGAM RNA, binds complementarily to one or more host target binding sites located in untranslated regions of VGAM85 host target RNA, herein designated VGAM HOST TARGET RNA. This complementary binding is due to the fact that the nucleotide sequence of VGAM85 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM85 RNA, herein designated VGAM RNA, may have a different number of host target binding sites in untranslated regions of a VGAM85 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM85 RNA, herein designated VGAM RNA, to host target binding sites on VGAM85 host target RNA, herein designated VGAM HOST TARGET RNA, such as BINDING SITE I, BINDING SITE II and BINDING SITE III, inhibits translation of VGAM85 host target RNA into VGAM85 host target protein, herein designated VGAM HOST TARGET PROTEIN. VGAM host target protein is therefore outlined by a broken line.

It is appreciated that VGAM85 host target gene, herein designated VGAM HOST TARGET GENE, in fact represents a plurality of VGAM85 host target genes. The mRNA of each one of this plurality of VGAM85 host target genes comprises one or more host target binding sites, each having a nucleotide sequence which is at least partly complementary to VGAM85 RNA, herein designated VGAM RNA, and which when bound by VGAM85 RNA causes inhibition of translation of respective one or more VGAM85 host target proteins.

It is further appreciated by one skilled in the art that the mode of translational inhibition illustrated by FIG. 1 with specific reference to translational inhibition exerted by VGAM85 gene, herein designated VGAM GENE, on one or more VGAM85 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM85 is inhibition of expression of host target genes, as part of a novel viral mechanism of attacking a host. Accordingly, utilities of VGAM85 include diagnosis, prevention and treatment of viral infection by Vaccinia Virus. Specific functions, and accordingly utilities, of VGAM85 correlate with, and may be deduced from, the identity of the host target genes which VGAM85 binds and inhibits, and the function of these host target genes, as elaborated hereinbelow.

Nucleotide sequences of the VGAM85 precursor RNA, herein designated VGAM PRECURSOR RNA, and of the ‘diced’ VGAM85 RNA, herein designated VGAM RNA, and a schematic representation of the secondary folding of VGAM85 folded precursor RNA, herein designated VGAM FOLDED PRECURSOR RNA, of VGAM85 are further described hereinbelow with reference to Table 1.

Nucleotide sequences of host target binding sites, such as BINDING SITE-I, BINDING SITE-II and BINDING SITE-III of FIG. 1, found on VGAM85 host target RNA, and schematic representation of the complementarity of each of these host target binding sites to VGAM85 RNA, herein designated VGAM RNA, are described hereinbelow with reference to Table 2.

As mentioned hereinabove with reference to FIG. 1, a function of VGAM85 gene, herein designated VGAM is inhibition of expression of VGAM85 target genes. It is appreciated that specific functions, and accordingly utilities, of VGAM85 correlate with, and may be deduced from, the identity of the target genes which VGAM85 binds and inhibits, and the function of these target genes, as elaborated hereinbelow.

Coagulation Factor II (thrombin) Receptor-like 3 (F2RL3, Accession NM_{—}003950) is a VGAM85 host target gene. F2RL3 BINDING SITE is HOST TARGET binding site found in the 3′ untranslated region of mRNA encoded by F2RL3, corresponding to a HOST TARGET binding site such as BINDING SITE I, BINDING SITE II or BINDING SITE III. Table 2 illustrates the complementarity of the nucleotide sequences of F2RL3 BINDING SITE, designated SEQ ID: 1074, to the nucleotide sequence of VGAM85 RNA, herein designated VGAM RNA, also designated SEQ ID:420.

A function of VGAM85 is therefore inhibition of Coagulation Factor II (thrombin) Receptor-like 3 (F2RL3, Accession NM_{—}003950), a gene which Protease-activated receptor 4; G protein-coupled receptor that increases phosphoinositide hydrolysis. Accordingly, utilities of VGAM85 include diagnosis, prevention and treatment of diseases and clinical conditions associated with F2RL3. The function of F2RL3 has been established by previous studies. Protease-activated receptors 1 (PARL; 187930), 2 (PAR2; 600933), and 3 (PAR3; 601919) are members of a unique G protein-coupled receptor family. They are characterized by a tethered peptide ligand at the extracellular amino terminus that is generated by minor proteolysis. Xu et al. (1998) identified a partial cDNA sequence of a fourth member of this family, PAR4, in an expressed sequence tag (EST) database, and a full-length cDNA clone was isolated from a lymphoma Daudi cell cDNA library. The open reading frame coded for a 7-transmembrane domain protein of 385 amino acids with 33% amino acid sequence identity with PAR1-3. A putative protease cleavage site was identified within the extracellular amino terminus. Northern blot analysis showed that PAR4 mRNA is expressed in a number of human tissues, with high levels being present in lung, pancreas, thyroid, testis, and small intestine. By fluorescence in situ hybridization, Xu et al. (1998) mapped the PAR4 gene to 19p12. Animal model experiments lend further support to the function of F2RL3. Sambrano et al. (2001) demonstrated that platelets from Par4-deficient mice failed to change shape, mobilize calcium, secrete ATP, or aggregate in response to thrombin.

It is appreciated that the abovementioned animal model for F2RL3 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Xu, W.-F.; Andersen, H.; Whitmore, T. E.; Presnell, S. R.; Yee, D. P.; Ching, A.; Gilbert, T.; Davie, E. W.; Foster, D. C.: Cloning and characterization of human protease-activated receptor 4. Proc. Nat. Acad. Sci. 95: 6642-6646, 1998.; and
Sambrano, G. R.; Weiss, E. J.; Zheng, Y.-W.; Huang, W.; Coughlin, S. R.: Role of thrombin signalling in platelets in haemostasis and thrombosis. Nature 413: 74-78, 2001.

Further studies establishing the function and utilities of F2RL3 are found in John Hopkins OMIM database record ID 602779, and in sited publications numbered 1755, 2132-213 and 1756 listed in the bibliography section hereinbelow, which are also hereby incorporated by reference. RAP1B, Member of RAS Oncogene Family (RAP1B, Accession NM_{—}015646) is another VGAM85 host target gene. RAP1B BINDING SITE is HOST TARGET binding site found in the 3′ untranslated region of mRNA encoded by RAP1B, corresponding to a HOST TARGET binding site such as BINDING SITE I, BINDING SITE II or BINDING SITE III. Table 2 illustrates the complementarity of the nucleotide sequences of RAP1B BINDING SITE, designated SEQ ID: 1642, to the nucleotide sequence of VGAM85 RNA, herein designated VGAM RNA, also designated SEQ ID:420.

Another function of VGAM85 is therefore inhibition of RAP1B, Member of RAS Oncogene Family (RAP1B, Accession NM_{—}015646), a gene which induces morphological reversion of a cell line transformed by a ras oncogene. Accordingly, utilities of VGAM85 include diagnosis, prevention and treatment of diseases and clinical conditions associated with RAP1B. The function of RAP1B has been established by previous studies. Three human cDNAs encoding ‘new’ RAS-related proteins, designated RAPLA, RAP1B, and RAP2, were isolated by Pizon et al. (1988). These proteins share approximately 50% amino acid identity with the classical RAS proteins and have numerous structural features in common. The most striking difference between the RAP and RAS proteins resides in their 61st amino acid: glutamine in RAS is replaced by threonine in RAP proteins. Animal model experiments lend further support to the function of RAP1B. Using mice transgenic for constitutive expression of Rap1a within the T cell lineage, Sebzda et al. (2002) found that instead of anergy, these T cells showed enhanced T cell receptor-mediated responses, both in thymocytes and in mature T cells. In addition, Rap1a activation induces strong activation of beta-1 (OMIM Ref. No. 135630) and beta-2 (OMIM Ref. No. 600065) integrins. The authors concluded that Rap1a positively influences T cells by augmenting their responses and directing integrin activation.

It is appreciated that the abovementioned animal model for RAP1B is acknowledged by those skilled in the art as a scientifically valid animal model, as can be further appreciated from the publications sited hereinbelow.

Full details of the abovementioned studies are described in the following publications, the disclosure of which are hereby incorporated by reference:
Pizon, V.; Chardin, P.; Lerosey, I.; Olofsson, B.; Tavitian, A.: Human cDNAs RAP1 and RAP2 homologous to the Drosophila gene Dras3 encode proteins closely related to ras in the ‘effector’ region. Oncogene 3: 201-204, 1988.; and
Kitayama, H.; Sugimoto, Y.; Matsuzaki, T.; Ikawa, Y.; Noda, M.: A ras-related gene with transformation suppressor activity. Cell 56: 77-84, 1989. PubMed ID: 2642744 9. Sebzda, E.; Brac.

Further studies establishing the function and utilities of RAP1B are found in John Hopkins OMIM database record ID 179530, and in sited publications numbered listed in the bibliography section hereinbelow, which are also hereby incorporated by reference. Solute Carrier Family 6 (neurotransmitter transporter, betaine/GABA), Member 12 (SLC6A12, Accession NM_{—}003044) is another VGAM85 host target gene. SLC6A12 BINDING SITE is HOST TARGET binding site found in the 3′ untranslated region of mRNA encoded by SLC6A12, corresponding to a HOST TARGET binding site such as BINDING SITE I, BINDING SITE II or BINDING SITE III. Table 2 illustrates the complementarity of the nucleotide sequences of SLC6A12 BINDING SITE, designated SEQ ID:988, to the nucleotide sequence of VGAM85 RNA, herein designated VGAM RNA, also designated SEQ ID:420.

Another function of VGAM85 is therefore inhibition of Solute Carrier Family 6 (neurotransmitter transporter, betaine/GABA), Member 12 (SLC6A12, Accession NM_{—}003044), a gene which transports betaine and gaba. Accordingly, utilities of VGAM85 include diagnosis, prevention and treatment of diseases and clinical conditions associated with SLC6A12. The function of SLC6A12 has been established by previous studies. Yamauchi et al. (1992) stated that Madin-Darby canine kidney (MDCK) cells accumulate betaine when cultured in hypertonic media. They isolated an MDCK cell cDNA encoding a renal betaine transporter and designated it BGT1. When expressed in Xenopus oocytes, the BGT1 protein exhibited chloride- and sodium-dependent transport of both betaine and the neurotransmitter GABA. Northern blot analysis revealed that BGT1 expression is limited to the canine kidney medulla and is induced in MDCK cells by hypertonicity. Using the canine BGT1 sequence, Rasola et al. (1995) isolated a cDNA from a kidney library encoding the human homolog. The predicted 614-amino acid human protein has the typical structure of neurotransmitter transporters, with 12 transmembrane domains and a large extracellular loop between the third and fourth transmembrane domains. Northern blot analysis indicated that BGT1 is expressed as several mRNAs in human kidney and other tissues. Borden et al. (1995) also isolated human BGT1 cDNAs and reported that the human protein shares 91% and 87% sequence identity with canine and mouse BGT1, respectively. Heterologous expression of human BGT1 in mammalian cells conferred high-affinity GABA uptake.

Full details of the abovementioned studies are described in the following publications, the disclosure of which are hereby incorporated by reference:
Yamauchi, A.; Uchida, S.; Kwon, H. M.; Preston, A. S.; Robey, R. B.; Garcia-Perez, A.; Burg, M. B.; Handler, J. S.: Cloning of a Na(+) and Cl(−)-dependent betaine transporter that is regulated by hypertonicity. J. Biol. Chem. 267: 649-652, 1992.; and
Rasola, A.; Galietta, L. J. V.; Barone, V.; Romeo, G.; Bagnasco, S.: Molecular cloning and functional characterization of a GABA/betaine transporter from human kidney. FEBS Lett. 373: 22.

Further studies establishing the function and utilities of SLC6A12 are found in John Hopkins OMIM database record ID 603080, and in sited publications numbered 238-240 listed in the bibliography section hereinbelow, which are also hereby incorporated by reference. Aldehyde Dehydrogenase 5 Family, Member A1 (succinate-semialdehyde dehydrogenase) (ALDH5A1, Accession NM_{—}001080) is another VGAM85 host target gene. ALDH5A1 BINDING SITE is HOST TARGET binding site found in the 3′ untranslated region of mRNA encoded by ALDH5A1, corresponding to a HOST TARGET binding site such as BINDING SITE I, BINDING SITE II or BINDING SITE III. Table 2 illustrates the complementarity of the nucleotide sequences of ALDH5A1 BINDING SITE, designated SEQ ID:799, to the nucleotide sequence of VGAM85 RNA, herein designated VGAM RNA, also designated SEQ ID:420.

Another function of VGAM85 is therefore inhibition of Aldehyde Dehydrogenase 5 Family, Member A1 (succinate-semialdehyde dehydrogenase) (ALDH5A1, Accession NM_{—}001080). Accordingly, utilities of VGAM85 include diagnosis, prevention and treatment of diseases and clinical conditions associated with ALDH5A1. Chromosome 20 Open Reading Frame 112 (C20orf112, Accession NM_{—}080616) is another VGAM85 host target gene. C20orf112 BINDING SITE is HOST TARGET binding site found in the 3′ untranslated region of mRNA encoded by C20orf112, corresponding to a HOST TARGET binding site such as BINDING SITE I, BINDING SITE II or BINDING SITE III. Table 2 illustrates the complementarity of the nucleotide sequences of C20orf112 BINDING SITE, designated SEQ ID:2379, to the nucleotide sequence of VGAM85 RNA, herein designated VGAM RNA, also designated SEQ ID:420.

Another function of VGAM85 is therefore inhibition of Chromosome 20 Open Reading Frame 112 (C20orf112, Accession NM_{—}080616). Accordingly, utilities of VGAM85 include diagnosis, prevention and treatment of diseases and clinical conditions associated with C20orf112. Dicer1, Dcr-1 Homolog (Drosophila) (DICER1, Accession NM_{—}030621) is another VGAM85 host target gene. DICER1 BINDING SITE is HOST TARGET binding site found in the 3′ untranslated region of mRNA encoded by DICER1, corresponding to a HOST TARGET binding site such as BINDING SITE I, BINDING SITE II or BINDING SITE III. Table 2 illustrates the complementarity of the nucleotide sequences of DICER1 BINDING SITE, designated SEQ ID:2152, to the nucleotide sequence of VGAM85 RNA, herein designated VGAM RNA, also designated SEQ ID:420.

Another function of VGAM85 is therefore inhibition of Dicer1, Dcr-1 Homolog (Drosophila) (DICER1, Accession NM_{—}030621). Accordingly, utilities of VGAM85 include diagnosis, prevention and treatment of diseases and clinical conditions associated with DICER1. DKFZP566G1424 (Accession XM_{—}097771) is another VGAM85 host target gene. DKFZP566G1424 BINDING SITE is HOST TARGET binding site found in the 5′ untranslated region of mRNA encoded by DKFZP566G1424, corresponding to a HOST TARGET binding site such as BINDING SITE I, BINDING SITE II or BINDING SITE III. Table 2 illustrates the complementarity of the nucleotide sequences of DKFZP566G1424 BINDING SITE, designated SEQ ID:3320, to the nucleotide sequence of VGAM85 RNA, herein designated VGAM RNA, also designated SEQ ID:420.

Another function of VGAM85 is therefore inhibition of DKFZP566G1424 (Accession XM_{—}097771). Accordingly, utilities of VGAM85 include diagnosis, prevention and treatment of diseases and clinical conditions associated with DKFZP566G1424. FLJ10858 (Accession NM_{—}018248) is another VGAM85 host target gene. FLJ10858 BINDING SITE is HOST TARGET binding site found in the 3′ untranslated region of mRNA encoded by FLJ10858, corresponding to a HOST TARGET binding site such as BINDING SITE I, BINDING SITE II or BINDING SITE III. Table 2 illustrates the complementarity of the nucleotide sequences of FLJ10858 BINDING SITE, designated SEQ ID:1805, to the nucleotide sequence of VGAM85 RNA, herein designated VGAM RNA, also designated SEQ ID:420.

Another function of VGAM85 is therefore inhibition of FLJ10858 (Accession NM_{—}018248). Accordingly, utilities of VGAM85 include diagnosis, prevention and treatment of diseases and clinical conditions associated with FLJ10858. FLJ13265 (Accession NM_{—}024877) is another VGAM85 host target gene. FLJ13265 BINDING SITE is HOST TARGET binding site found in the 3′ untranslated region of mRNA encoded by FLJ13265, corresponding to a HOST TARGET binding site such as BINDING SITE I, BINDING SITE II or BINDING SITE III. Table 2 illustrates the complementarity of the nucleotide sequences of FLJ13265 BINDING SITE, designated SEQ ID:2100, to the nucleotide sequence of VGAM85 RNA, herein designated VGAM RNA, also designated SEQ ID:420.

Another function of VGAM85 is therefore inhibition of FLJ13265 (Accession NM_{—}024877). Accordingly, utilities of VGAM85 include diagnosis, prevention and treatment of diseases and clinical conditions associated with FLJ13265. FLJ22282 (Accession NM_{—}024792) is another VGAM85 host target gene. FLJ22282 BINDING SITE is HOST TARGET binding site found in the 3′ untranslated region of mRNA encoded by FLJ22282, corresponding to a HOST TARGET binding site such as BINDING SITE I, BINDING SITE II or BINDING SITE III. Table 2 illustrates the complementarity of the nucleotide sequences of FLJ22282 BINDING SITE, designated SEQ ID:2089, to the nucleotide sequence of VGAM85 RNA, herein designated VGAM RNA, also designated SEQ ID:420.

Another function of VGAM85 is therefore inhibition of FLJ22282 (Accession NM_{—}024792). Accordingly, utilities of VGAM85 include diagnosis, prevention and treatment of diseases and clinical conditions associated with FLJ22282. GBTS1 (Accession NM_{—}145173) is another VGAM85 host target gene. GBTS1 BINDING SITE is HOST TARGET binding site found in the 3′ untranslated region of mRNA encoded by GBTS1, corresponding to a HOST TARGET binding site such as BINDING SITE I, BINDING SITE II or BINDING SITE III. Table 2 illustrates the complementarity of the nucleotide sequences of GBTS1 BINDING SITE, designated SEQ ID:2516, to the nucleotide sequence of VGAM85 RNA, herein designated VGAM RNA, also designated SEQ ID:420.

Another function of VGAM85 is therefore inhibition of GBTS1 (Accession NM_{—}145173). Accordingly, utilities of VGAM85 include diagnosis, prevention and treatment of diseases and clinical conditions associated with GBTS1. KIAA0193 (Accession NM_{—}014766) is another VGAM85 host target gene. KIAA0193 BINDING SITE is HOST TARGET binding site found in the 3′ untranslated region of mRNA encoded by KIAA0193, corresponding to a HOST TARGET binding site such as BINDING SITE I, BINDING SITE II or BINDING SITE III. Table 2 illustrates the complementarity of the nucleotide sequences of KIAA0193 BINDING SITE, designated SEQ ID:1545, to the nucleotide sequence of VGAM85 RNA, herein designated VGAM RNA, also designated SEQ ID:420.

Another function of VGAM85 is therefore inhibition of KIAA0193 (Accession NM_{—}014766). Accordingly, utilities of VGAM85 include diagnosis, prevention and treatment of diseases and clinical conditions associated with KIAA0193. KIAA1030 (Accession XM_{—}167789) is another VGAM85 host target gene. KIAA1030 BINDING SITE is HOST TARGET binding site found in the 3′ untranslated region of mRNA encoded by KIAA1030, corresponding to a HOST TARGET binding site such as BINDING SITE I, BINDING SITE II or BINDING SITE III. Table 2 illustrates the complementarity of the nucleotide sequences of KIAA1030 BINDING SITE, designated SEQ ID:3597, to the nucleotide sequence of VGAM85 RNA, herein designated VGAM RNA, also designated SEQ ID:420.

Another function of VGAM85 is therefore inhibition of KIAA1030 (Accession XM_{—}167789). Accordingly, utilities of VGAM85 include diagnosis, prevention and treatment of diseases and clinical conditions associated with KIAA1030. Olfactory Receptor, Family 7, Sub family C, Member 1 (OR7C1, Accession NM_{—}017506) is another VGAM85 host target gene. OR7C1 BINDING SITE is HOST TARGET binding site found in the 3′ untranslated region of mRNA encoded by OR7C1, corresponding to a HOST TARGET binding site such as BINDING SITE I, BINDING SITE II or BINDING SITE III. Table 2 illustrates the complementarity of the nucleotide sequences of OR7C1 BINDING SITE, designated SEQ ID:1719, to the nucleotide sequence of VGAM85 RNA, herein designated VGAM RNA, also designated SEQ ID:420.

Another function of VGAM85 is therefore inhibition of Olfactory Receptor, Family 7, Sub family C, Member 1 (OR7C1, Accession NM_{—}017506). Accordingly, utilities of VGAM85 include diagnosis, prevention and treatment of diseases and clinical conditions associated with OR7C1. PRO2214 (Accession NM_{—}018517) is another VGAM85 host target gene. PRO2214 BINDING SITE is HOST TARGET binding site found in the 3′ untranslated region of mRNA encoded by PRO2214, corresponding to a HOST TARGET binding site such as BINDING SITE I, BINDING SITE II or BINDING SITE III. Table 2 illustrates the complementarity of the nucleotide sequences of PRO2214 BINDING SITE, designated SEQ ID:1834, to the nucleotide sequence of VGAM85 RNA, herein designated VGAM RNA, also designated SEQ ID:420.

Another function of VGAM85 is therefore inhibition of PRO2214 (Accession NM_{—}018517). Accordingly, utilities of VGAM85 include diagnosis, prevention and treatment of diseases and clinical conditions associated with PRO2214. Smith-Magenis Syndrome Chromosome Region, Candidate 5 (SMCR5, Accession NM_{—}144774) is another VGAM85 host target gene. SMCR5 BINDING SITE is HOST TARGET binding site found in the 3′ untranslated region of mRNA encoded by SMCR5, corresponding to a HOST TARGET binding site such as BINDING SITE I, BINDING SITE II or BINDING SITE III. Table 2 illustrates the complementarity of the nucleotide sequences of SMCR5 BINDING SITE, designated SEQ ID:2502, to the nucleotide sequence of VGAM85 RNA, herein designated VGAM RNA, also designated SEQ ID:420.

Another function of VGAM85 is therefore inhibition of Smith-Magenis Syndrome Chromosome Region, Candidate 5 (SMCR5, Accession NM_{—}144774). Accordingly, utilities of VGAM85 include diagnosis, prevention and treatment of diseases and clinical conditions associated with SMCR5. SSH1 (Accession NM_{—}018984) is another VGAM85 host target gene. SSH1 BINDING SITE is HOST TARGET binding site found in the 3′ untranslated region of mRNA encoded by SSH1, corresponding to a HOST TARGET binding site such as BINDING SITE I, BINDING SITE II or BINDING SITE III. Table 2 illustrates the complementarity of the nucleotide sequences of SSH1 BINDING SITE, designated SEQ ID:1869, to the nucleotide sequence of VGAM85 RNA, herein designated VGAM RNA, also designated SEQ ID:420.

Another function of VGAM85 is therefore inhibition of SSH1 (Accession NM_{—}018984). Accordingly, utilities of VGAM85 include diagnosis, prevention and treatment of diseases and clinical conditions associated with SSH1. TERA (Accession NM_{—}021238) is another VGAM85 host target gene. TERA BINDING SITE is HOST TARGET binding site found in the 3′ untranslated region of mRNA encoded by TERA, corresponding to a HOST TARGET binding site such as BINDING SITE I, BINDING SITE II or BINDING SITE III. Table 2 illustrates the complementarity of the nucleotide sequences of TERA BINDING SITE, designated SEQ ID:1948, to the nucleotide sequence of VGAM85 RNA, herein designated VGAM RNA, also designated SEQ ID:420.

Another function of VGAM85 is therefore inhibition of TERA (Accession NM_{—}021238). Accordingly, utilities of VGAM85 include diagnosis, prevention and treatment of diseases and clinical conditions associated with TERA. Williams-Beuren Syndrome Chromosome Region 23 (WBSCR23, Accession NM_{—}025042) is another VGAM85 host target gene. WBSCR23 BINDING SITE is HOST TARGET binding site found in the 5′ untranslated region of mRNA encoded by WBSCR23, corresponding to a HOST TARGET binding site such as BINDING SITE I, BINDING SITE II or BINDING SITE III. Table 2 illustrates the complementarity of the nucleotide sequences of WBSCR23 BINDING SITE, designated SEQ ID:2131, to the nucleotide sequence of VGAM85 RNA, herein designated VGAM RNA, also designated SEQ ID:420.

Another function of VGAM85 is therefore inhibition of Williams-Beuren Syndrome Chromosome Region 23 (WBSCR23, Accession NM_{—}025042). Accordingly, utilities of VGAM85 include diagnosis, prevention and treatment of diseases and clinical conditions associated with WBSCR23. LOC134637 (Accession XM_{—}059727) is another VGAM85 host target gene. LOC134637 BINDING SITE is HOST TARGET binding site found in the 3′ untranslated region of mRNA encoded by LOC134637, corresponding to a HOST TARGET binding site such as BINDING SITE I, BINDING SITE II or BINDING SITE III. Table 2 illustrates the complementarity of the nucleotide sequences of LOC134637 BINDING SITE, designated SEQ ID:3017, to the nucleotide sequence of VGAM85 RNA, herein designated VGAM RNA, also designated SEQ ID:420.

Another function of VGAM85 is therefore inhibition of LOC134637 (Accession XM_{—}059727). Accordingly, utilities of VGAM85 include diagnosis, prevention and treatment of diseases and clinical conditions associated with LOC134637. LOC146745 (Accession XM_{—}085577) is another VGAM85 host target gene. LOC146745 BINDING SITE is HOST TARGET binding site found in the 5′ untranslated region of mRNA encoded by LOC146745, corresponding to a HOST TARGET binding site such as BINDING SITE I, BINDING SITE II or BINDING SITE III. Table 2 illustrates the complementarity of the nucleotide sequences of LOC146745 BINDING SITE, designated SEQ ID:3098, to the nucleotide sequence of VGAM85 RNA, herein designated VGAM RNA, also designated SEQ ID:420.

Another function of VGAM85 is therefore inhibition of LOC146745 (Accession XM_{—}085577). Accordingly, utilities of VGAM85 include diagnosis, prevention and treatment of diseases and clinical conditions associated with LOC146745. LOC153416 (Accession XM_{—}018473) is another VGAM85 host target gene. LOC153416 BINDING SITE is HOST TARGET binding site found in the 3′ untranslated region of mRNA encoded by LOC153416, corresponding to a HOST TARGET binding site such as BINDING SITE I, BINDING SITE II or BINDING SITE III. Table 2 illustrates the complementarity of the nucleotide sequences of LOC153416 BINDING SITE, designated SEQ ID:2573, to the nucleotide sequence of VGAM85 RNA, herein designated VGAM RNA, also designated SEQ ID:420.

Another function of VGAM85 is therefore inhibition of LOC153416 (Accession XM_{—}018473). Accordingly, utilities of VGAM85 include diagnosis, prevention and treatment of diseases and clinical conditions associated with LOC153416. LOC157867 (Accession XM_{—}098831) is another VGAM85 host target gene. LOC157867 BINDING SITE is HOST TARGET binding site found in the 5′ untranslated region of mRNA encoded by LOC157867, corresponding to a HOST TARGET binding site such as BINDING SITE I, BINDING SITE II or BINDING SITE III. Table 2 illustrates the complementarity of the nucleotide sequences of LOC157867 BINDING SITE, designated SEQ ID:3383, to the nucleotide sequence of VGAM85 RNA, herein designated VGAM RNA, also designated SEQ ID:420.

Another function of VGAM85 is therefore inhibition of LOC157867 (Accession XM_{—}098831). Accordingly, utilities of VGAM85 include diagnosis, prevention and treatment of diseases and clinical conditions associated with LOC157867. LOC201243 (Accession XM_{—}113935) is another VGAM85 host target gene. LOC201243 BINDING SITE is HOST TARGET binding site found in the 5′ untranslated region of mRNA encoded by LOC201243, corresponding to a HOST TARGET binding site such as BINDING SITE I, BINDING SITE II or BINDING SITE III. Table 2 illustrates the complementarity of the nucleotide sequences of LOC201243 BINDING SITE, designated SEQ ID:3425, to the nucleotide sequence of VGAM85 RNA, herein designated VGAM RNA, also designated SEQ ID:420.

Another function of VGAM85 is therefore inhibition of LOC201243 (Accession XM_{—}113935). Accordingly, utilities of VGAM85 include diagnosis, prevention and treatment of diseases and clinical conditions associated with LOC201243. FIG. 1 further provides a conceptual description of a novel bioinformatically detected viral gene of the present invention, referred to here as Viral Genomic Address Messenger 86 (VGAM86) viral gene, which modulates expression of respective host target genes thereof, the function and utility of which host target genes is known in the art.

VGAM86 is a novel bioinformatically detected regulatory, non protein coding, viral micro RNA (miRNA) gene. The method by which VGAM86 was detected is described hereinabove with reference to FIGS. 1-8.

VGAM86 gene, herein designated VGAM GENE, is a viral gene contained in the genome of Vaccinia Virus. VGAM86 host target gene, herein designated VGAM HOST TARGET GENE, is a human gene contained in the human genome.

VGAM86 gene encodes a VGAM86 precursor RNA, herein designated VGAM PRECURSOR RNA. Similar to other miRNA genes, and unlike most ordinary genes, VGAM86 precursor RNA does not encode a protein. A nucleotide sequence identical or highly similar to the nucleotide sequence of VGAM86 precursor RNA is designated SEQ ID:72, and is provided hereinbelow with reference to the sequence listing part. Nucleotide sequence SEQ ID:72 is located at position 25950 relative to the genome of Vaccinia Virus.

VGAM86 precursor RNA folds onto itself, forming VGAM86 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM86 folded precursor RNA into VGAM86 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM86 RNA is designated SEQ ID:421, and is provided hereinbelow with reference to the sequence listing part.

VGAM86 host target gene, herein designated VGAM HOST TARGET GENE, encodes a corresponding messenger RNA, VGAM86 host target RNA, herein designated VGAM HOST TARGET RNA. VGAM86 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM86 RNA, herein designated VGAM RNA, binds complementarily to one or more host target binding sites located in untranslated regions of VGAM86 host target RNA, herein designated VGAM HOST TARGET RNA. This complementary binding is due to the fact that the nucleotide sequence of VGAM86 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM86 RNA, herein designated VGAM RNA, may have a different number of host target binding sites in untranslated regions of a VGAM86 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM86 RNA, herein designated VGAM RNA, to host target binding sites on VGAM86 host target RNA, herein designated VGAM HOST TARGET RNA, such as BINDING SITE I, BINDING SITE II and BINDING SITE III, inhibits translation of VGAM86 host target RNA into VGAM86 host target protein, herein designated VGAM HOST TARGET PROTEIN. VGAM host target protein is therefore outlined by a broken line.

It is appreciated that VGAM86 host target gene, herein designated VGAM HOST TARGET GENE, in fact represents a plurality of VGAM86 host target genes. The mRNA of each one of this plurality of VGAM86 host target genes comprises one or more host target binding sites, each having a nucleotide sequence which is at least partly complementary to VGAM86 RNA, herein designated VGAM RNA, and which when bound by VGAM86 RNA causes inhibition of translation of respective one or more VGAM86 host target proteins.

It is further appreciated by one skilled in the art that the mode of translational inhibition illustrated by FIG. 1 with specific reference to translational inhibition exerted by VGAM86 gene, herein designated VGAM GENE, on one or more VGAM86 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM86 is inhibition of expression of host target genes, as part of a novel viral mechanism of attacking a host. Accordingly, utilities of VGAM86 include diagnosis, prevention and treatment of viral infection by Vaccinia Virus. Specific functions, and accordingly utilities, of VGAM86 correlate with, and may be deduced from, the identity of the host target genes which VGAM86 binds and inhibits, and the function of these host target genes, as elaborated hereinbelow.

Nucleotide sequences of the VGAM86 precursor RNA, herein designated VGAM PRECURSOR RNA, and of the ‘diced’ VGAM86 RNA, herein designated VGAM RNA, and a schematic representation of the secondary folding of VGAM86 folded precursor RNA, herein designated VGAM FOLDED PRECURSOR RNA, of VGAM86 are further described hereinbelow with reference to Table 1.

Nucleotide sequences of host target binding sites, such as BINDING SITE-I, BINDING SITE-II and BINDING SITE-III of FIG. 1, found on VGAM86 host target RNA, and schematic representation of the complementarity of each of these host target binding sites to VGAM86 RNA, herein designated VGAM RNA, are described hereinbelow with reference to Table 2.

As mentioned hereinabove with reference to FIG. 1, a function of VGAM86 gene, herein designated VGAM is inhibition of expression of VGAM86 target genes. It is appreciated that specific functions, and accordingly utilities, of VGAM86 correlate with, and may be deduced from, the identity of the target genes which VGAM86 binds and inhibits, and the function of these target genes, as elaborated hereinbelow.

Oxidative-stress Responsive 1 (OSR1, Accession NM_{—}005109) is a VGAM86 host target gene. OSR1 BINDING SITE is HOST TARGET binding site found in the 3′ untranslated region of mRNA encoded by OSR1, corresponding to a HOST TARGET binding site such as BINDING SITE I, BINDING SITE II or BINDING SITE III. Table 2 illustrates the complementarity of the nucleotide sequences of OSR1 BINDING SITE, designated SEQ ID:1188, to the nucleotide sequence of VGAM86 RNA, herein designated VGAM RNA, also designated SEQ ID:421.

A function of VGAM86 is therefore inhibition of Oxidative-stress Responsive 1 (OSR1, Accession NM_{—}005109), a gene which mediats stress-activated signals. Accordingly, utilities of VGAM86 include diagnosis, prevention and treatment of diseases and clinical conditions associated with OSR1. The function of OSR1 has been established by previous studies. The 3p22-p21.3 chromosomal region is one of 3 regions of 3p that is commonly deleted in various carcinomas. By analyzing a cloned segment from this region, Tamari et al. (1999) identified a novel gene that they designated OSR1 (oxidative stress-responsive-1) because the predicted 527-amino acid protein shares 39% identity with Ste20/oxidant stress-response kinase-1 (OMIM Ref. No. 602255). The OSR1 gene contains 18 exons and spans approximately 90 kb. Northern blot analysis revealed that OSR1 was expressed as a 4.6-kb major transcript in all tissues tested. A less abundant 7.5-kb mRNA was detected in heart and skeletal muscle. Daigo et al. (1999) reported that the OSR1 gene is located between the OCTL1 (OMIM Ref. No. 604047) and MYD88 (OMIM Ref. No. 602170) genes on 3p22-p21.3.

Full details of the abovementioned studies are described in the following publications, the disclosure of which are hereby incorporated by reference:
Tamari, M.; Daigo, Y.; Nakamura, Y.: Isolation and characterization of a novel serine threonine kinase gene on chromosome 3q22-21.3. J. Hum. Genet. 44: 116-120, 1999.; and
Daigo, Y.; Isomura, M.; Nishiwaki, T.; Tamari, M.; Ishikawa, S.; Kai, M.; Murata, Y.; Takeuchi, K.; Yamane, Y.; Hayashi, R.; Minami, M.; Fujino, M. A.; Hojo, Y.; Uchiyama, I.; Takagi, T.;.

Further studies establishing the function and utilities of OSR1 are found in John Hopkins OMIM database record ID 604046, and in sited publications numbered 2027 listed in the bibliography section hereinbelow, which are also hereby incorporated by reference. Paired Basic Amino Acid Cleaving System 4 (PACE4, Accession NM_{—}138325) is another VGAM86 host target gene. PACE4 BINDING SITE is HOST TARGET binding site found in the 5′ untranslated region of mRNA encoded by PACE4, corresponding to a HOST TARGET binding site such as BINDING SITE I, BINDING SITE II or BINDING SITE III. Table 2 illustrates the complementarity of the nucleotide sequences of PACE4 BINDING SITE, designated SEQ ID:2435, to the nucleotide sequence of VGAM86 RNA, herein designated VGAM RNA, also designated SEQ ID:421.

Another function of VGAM86 is therefore inhibition of Paired Basic Amino Acid Cleaving System 4 (PACE4, Accession NM_{—}138325), a gene which processes hormone precursors by cleaving paired basic amino acids. Accordingly, utilities of VGAM86 include diagnosis, prevention and treatment of diseases and clinical conditions associated with PACE4. The function of PACE4 has been established by previous studies. Using PCR methods, Kiefer et al. (1991) identified a second human subtilisin-like protease gene on chromosome 15. PCR primers were designed to be specific for the sub family of eukaryotic subtilisin-like proteases with specificity for paired basic amino acid residue processing motifs. The gene encoding this protease, designated PACE4, also encoded a smaller subtilisin-related polypeptide derived by alternative mRNA splicing. As with the product of the PACE gene (OMIM Ref. No. 136950), the tissue distribution of PACE4 was widespread, with comparatively higher levels in the liver. By in situ hybridization using isolated cosmid clones, Kiefer et al. (1991) mapped the PACE4 gene to chromosome 15 in close proximity to the PACE gene at 15q25-q26. Double labeling in situ hybridization suggested that the 2 genes are within 5 megabases of each other. Mbikay et al. (1995) mapped the gene for PACE4 (Pcsk6) to mouse chromosome 7 by RFLP analysis of a DNA panel from an interspecific backcross. It was located at a distance of 13 cM from the Pcsk3 locus, which specifies furin (OMIM Ref. No. 136950), another member of this family of enzymes previously mapped to mouse chromosome 7. This is in concordance with the known close proximity of these 2 loci in the homologous region on human 15q25-qter. Pcsk3 and Pcsk6 map to a region of mouse chromosome 7 that has been associated cytogenetically with postnatal lethality in maternal disomy, suggesting that these genes may be imprinted.

Full details of the abovementioned studies are described in the following publications, the disclosure of which are hereby incorporated by reference:
Kiefer, M. C.; Tucker, J. E.; Joh, R.; Landsberg, K. E.; Saltman, D.; Barr, P. J.: Identification of a second human subtilisin-like protease gene in the fes/fps region of chromosome 15. DNA Cell Biol. 10: 757-769, 1991.; and
Mbikay, M.; Seidah, N. G.; Chretien, M.; Simpson, E. M.: Chromosomal assignment of the genes for proprotein convertases PC4, PC5, and PACE 4 in mouse and human. Genomics 26: 123-129, 19.

Further studies establishing the function and utilities of PACE4 are found in John Hopkins OMIM database record ID 167405, and in sited publications numbered 2365 listed in the bibliography section hereinbelow, which are also hereby incorporated by reference. KIAA1462 (Accession XM_{—}166132) is another VGAM86 host target gene. KIAA1462 BINDING SITE is HOST TARGET binding site found in the 3′ untranslated region of mRNA encoded by KIAA1462, corresponding to a HOST TARGET binding site such as BINDING SITE I, BINDING SITE II or BINDING SITE III. Table 2 illustrates the complementarity of the nucleotide sequences of KIAA1462 BINDING SITE, designated SEQ ID:3522, to the nucleotide sequence of VGAM86 RNA, herein designated VGAM RNA, also designated SEQ ID:421.

Another function of VGAM86 is therefore inhibition of KIAA1462 (Accession XM_{—}166132). Accordingly, utilities of VGAM86 include diagnosis, prevention and treatment of diseases and clinical conditions associated with KIAA1462. LOC145945 (Accession XM_{—}096908) is another VGAM86 host target gene. LOC145945 BINDING SITE is HOST TARGET binding site found in the 3′ untranslated region of mRNA encoded by LOC145945, corresponding to a HOST TARGET binding site such as BINDING SITE I, BINDING SITE II or BINDING SITE III. Table 2 illustrates the complementarity of the nucleotide sequences of LOC145945 BINDING SITE, designated SEQ ID:3280, to the nucleotide sequence of VGAM86 RNA, herein designated VGAM RNA, also designated SEQ ID:421.

Another function of VGAM86 is therefore inhibition of LOC145945 (Accession XM_{—}096908). Accordingly, utilities of VGAM86 include diagnosis, prevention and treatment of diseases and clinical conditions associated with LOC145945. LOC219401 (Accession XM_{—}166706) is another VGAM86 host target gene. LOC219401 BINDING SITE is HOST TARGET binding site found in the 5′ untranslated region of mRNA encoded by LOC219401, corresponding to a HOST TARGET binding site such as BINDING SITE I, BINDING SITE II or BINDING SITE III. Table 2 illustrates the complementarity of the nucleotide sequences of LOC219401 BINDING SITE, designated SEQ ID:3580, to the nucleotide sequence of VGAM86 RNA, herein designated VGAM RNA, also designated SEQ ID:421.

Another function of VGAM86 is therefore inhibition of LOC219401 (Accession XM_{—}166706). Accordingly, utilities of VGAM86 include diagnosis, prevention and treatment of diseases and clinical conditions associated with LOC219401. FIG. 1 further provides a conceptual description of a novel bioinformatically detected viral gene of the present invention, referred to here as Viral Genomic Address Messenger 87 (VGAM87) viral gene, which modulates expression of respective host target genes thereof, the function and utility of which host target genes is known in the art.

VGAM87 is a novel bioinformatically detected regulatory, non protein coding, viral micro RNA (miRNA) gene. The method by which VGAM87 was detected is described hereinabove with reference to FIGS. 1-8.

VGAM87 gene, herein designated VGAM GENE, is a viral gene contained in the genome of Vaccinia Virus. VGAM87 host target gene, herein designated VGAM HOST TARGET GENE, is a human gene contained in the human genome.

VGAM87 gene encodes a VGAM87 precursor RNA, herein designated VGAM PRECURSOR RNA. Similar to other miRNA genes, and unlike most ordinary genes, VGAM87 precursor RNA does not encode a protein. A nucleotide sequence identical or highly similar to the nucleotide sequence of VGAM87 precursor RNA is designated SEQ ID:73, and is provided hereinbelow with reference to the sequence listing part. Nucleotide sequence SEQ ID:73 is located at position 28644 relative to the genome of Vaccinia Virus.

VGAM87 precursor RNA folds onto itself, forming VGAM87 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM87 folded precursor RNA into VGAM87 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM87 RNA is designated SEQ ID:422, and is provided hereinbelow with reference to the sequence listing part.

VGAM87 host target gene, herein designated VGAM HOST TARGET GENE, encodes a corresponding messenger RNA, VGAM87 host target RNA, herein designated VGAM HOST TARGET RNA. VGAM87 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM87 RNA, herein designated VGAM RNA, binds complementarily to one or more host target binding sites located in untranslated regions of VGAM87 host target RNA, herein designated VGAM HOST TARGET RNA. This complementary binding is due to the fact that the nucleotide sequence of VGAM87 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM87 RNA, herein designated VGAM RNA, may have a different number of host target binding sites in untranslated regions of a VGAM87 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM87 RNA, herein designated VGAM RNA, to host target binding sites on VGAM87 host target RNA, herein designated VGAM HOST TARGET RNA, such as BINDING SITE I, BINDING SITE II and BINDING SITE III, inhibits translation of VGAM87 host target RNA into VGAM87 host target protein, herein designated VGAM HOST TARGET PROTEIN. VGAM host target protein is therefore outlined by a broken line.

It is appreciated that VGAM87 host target gene, herein designated VGAM HOST TARGET GENE, in fact represents a plurality of VGAM87 host target genes. The mRNA of each one of this plurality of VGAM87 host target genes comprises one or more host target binding sites, each having a nucleotide sequence which is at least partly complementary to VGAM87 RNA, herein designated VGAM RNA, and which when bound by VGAM87 RNA causes inhibition of translation of respective one or more VGAM87 host target proteins.

It is further appreciated by one skilled in the art that the mode of translational inhibition illustrated by FIG. 1 with specific reference to translational inhibition exerted by VGAM87 gene, herein designated VGAM GENE, on one or more VGAM87 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM87 is inhibition of expression of host target genes, as part of a novel viral mechanism of attacking a host. Accordingly, utilities of VGAM87 include diagnosis, prevention and treatment of viral infection by Vaccinia Virus. Specific functions, and accordingly utilities, of VGAM87 correlate with, and may be deduced from, the identity of the host target genes which VGAM87 binds and inhibits, and the function of these host target genes, as elaborated hereinbelow.

Nucleotide sequences of the VGAM87 precursor RNA, herein designated VGAM PRECURSOR RNA, and of the ‘diced’ VGAM87 RNA, herein designated VGAM RNA, and a schematic representation of the secondary folding of VGAM87 folded precursor RNA, herein designated VGAM FOLDED PRECURSOR RNA, of VGAM87 are further described hereinbelow with reference to Table 1.

Nucleotide sequences of host target binding sites, such as BINDING SITE-I, BINDING SITE-II and BINDING SITE-III of FIG. 1, found on VGAM87 host target RNA, and schematic representation of the complementarity of each of these host target binding sites to VGAM87 RNA, herein designated VGAM RNA, are described hereinbelow with reference to Table 2.

As mentioned hereinabove with reference to FIG. 1, a function of VGAM87 gene, herein designated VGAM is inhibition of expression of VGAM87 target genes. It is appreciated that specific functions, and accordingly utilities, of VGAM87 correlate with, and may be deduced from, the identity of the target genes which VGAM87 binds and inhibits, and the function of these target genes, as elaborated hereinbelow.

ATP-binding Cassette, Sub-family A (ABC1), Member 1 (ABCA1, Accession NM_{—}005502) is a VGAM87 host target gene. ABCA1 BINDING SITE is HOST TARGET binding site found in the 3′ untranslated region of mRNA encoded by ABCA1, corresponding to a HOST TARGET binding site such as BINDING SITE I, BINDING SITE II or BINDING SITE III. Table 2 illustrates the complementarity of the nucleotide sequences of ABCA1 BINDING SITE, designated SEQ ID:1219, to the nucleotide sequence of VGAM87 RNA, herein designated VGAM RNA, also designated SEQ ID:422.

A function of VGAM87 is therefore inhibition of ATP-binding Cassette, Sub-family A (ABC1), Member 1 (ABCA1, Accession NM_{—}005502), a gene which camp-dependent and sulfonylurea-sensitive anion transporter. Accordingly, utilities of VGAM87 include diagnosis, prevention and treatment of diseases and clinical conditions associated with ABCA1. The function of ABCA1 has been established by previous studies. By a PCR-based approach, Luciani et al. (1994) identified 2 novel mammalian members of the family of ATP-binding cassette (ABC) transporters designated ABC1 and ABC2 (OMIM Ref. No. 600047). They belong to a group of traffic ATPases encoded as a single multifunctional protein, such as CFTR (OMIM Ref. No. 602421) and P-glycoproteins (see OMIM Ref. No. 171050). Both ABC1 and ABC2 are large, internally symmetrical molecules that contain complete information for a functional ‘channel-like’ structure, a feature typical of the mammalian transporters at the plasma membrane. In both ABC1 and ABC2, the 2 halves of the molecules do not share extensive sequence similarity, apart from the nucleotide binding domains. This feature, shared with CFTR and with MRP1 (OMIM Ref. No. 158343), is in contrast with the high similarity shown by the 2 halves of P-glycoproteins. The finding argues against internal gene duplication as the event giving rise to the symmetric structure and favors the alternative hypothesis of the fusion of 2 independently evolved genes encoding the 2 halves. Santamarina-Fojo et al. (2000) found that the ABCA1 gene spans 149 kb and contains 50 exons. They identified 62 repetitive Alu sequences in the 49 introns. Comparative analysis of the mouse and human ABCA1 promoter sequences identified specific regulatory elements that are evolutionarily conserved. Pullinger et al. (2000) analyzed the promoter region of ABCA1. They identified 7 putative SP1 (OMIM Ref. No. 189906)-binding sites, 4 sterol regulatory elements (SREs) similar to the SRE of the low density lipoprotein receptor (LDLR; 606945) promoter region, a CpG island, a possible weak TATA box, 2 distal CCAAT sequences, and binding sites for several other transcription factors.

Full details of the abovementioned studies are described in the following publications, the disclosure of which are hereby incorporated by reference:
Luciani, M. F.; Denizot, F.; Savary, S.; Mattei, M. G.; Chimini, G.: Cloning of two novel ABC transporters mapping on human chromosome 9. Genomics 21: 150-159, 1994.; and
Santamarina-Fojo, S.; Peterson, K.; Knapper, C.; Qiu, Y.; Freeman, L.; Cheng, J.-F.; Osorio, J.; Remaley, A.; Yang, X.-P.; Haudenschild, C.; Prades, C.; Chimini, G.; Blackmon, E.; Franc.

Further studies establishing the function and utilities of ABCA1 are found in John Hopkins OMIM database record ID 600046, and in sited publications numbered 2331-2157, 2332, 2333-2334, 1662, 2335-2339, 2346, 2341-2343, 2347, 2349, 2159-1759, 263 and 2160-1762 listed in the bibliography section hereinbelow, which are also hereby incorporated by reference. CD2-associated Protein (CD2AP, Accession NM_{—}012120) is another VGAM87 host target gene. CD2AP BINDING SITE is HOST TARGET binding site found in the 3′ untranslated region of mRNA encoded by CD2AP, corresponding to a HOST TARGET binding site such as BINDING SITE I, BINDING SITE II or BINDING SITE III. Table 2 illustrates the complementarity of the nucleotide sequences of CD2AP BINDING SITE, designated SEQ ID: 1408, to the nucleotide sequence of VGAM87 RNA, herein designated VGAM RNA, also designated SEQ ID:422.

Another function of VGAM87 is therefore inhibition of CD2-associated Protein (CD2AP, Accession NM_{—}012120), a gene which binds CAS ligand and may therefor involves in its growth regulatory pathway. Accordingly, utilities of VGAM87 include diagnosis, prevention and treatment of diseases and clinical conditions associated with CD2AP. The function of CD2AP has been established by previous studies. P130(Cas) (OMIM Ref. No. 602941) is a docking protein that is tyrosine-phosphorylated in response to a variety of extracellular stimuli, such as growth factors, cell-cell interaction, and cell-matrix interaction, and appears to play a critical role in the integrin-linked formation of focal complexes. To understand the growth regulatory pathway of p130(Cas), Kirsch et al. (1999) used the yeast 2-hybrid system to search for interacting molecules. They identified a human protein, which they called CMS for p130(Cas) ligand with multiple SH3 domains, as a direct binding protein of p130(Cas). CMS is a multifunctional adapter-type molecule, which is localized in the cytoplasm, membrane ruffles, and leading edges of cells. Its structure and colocalization with F-actin (see OMIM Ref. No. 102610) and p130(Cas) suggested a function as a scaffolding protein involved in the dynamic regulation of the actin cytoskeleton. The cDNA corresponding to CMS encodes a protein of 639 amino acids with a deduced molecular mass of approximately 70 kD. Amino acid analysis revealed that CMS contains in its N terminus 3 SH3 domains followed by a proline-rich region containing binding sites for SH3 domains. Putative actin-binding sites and a coiled-coil domain are located at the C terminus of the protein, which also contains a putative leucine zipper motif. CMS mRNA is ubiquitously expressed in adult and fetal human tissues as an approximately 5.4-kb transcript, as detected by Northern blot analysis. CMS induces vesicle formation and colocalizes with p130(Cas) and F-actin to membrane ruffles. It also associates with and is phosphorylated by tyrosine kinases. Kirsch et al. (1999) demonstrated that CMS is able to homodimerize through the coiled-coil domain located in its C terminus. There was no evidence for intermolecular or intramolecular binding via the SH3 domains and PXXP binding. Animal model experiments lend further support to the function of CD2AP. Shih et al. (1999) generated mice lacking CD2AP by targeted disruption. In CD2AP-deficient mice, immune function was compromised, but the mice died from renal failure at 6 to 7 weeks of age. In the kidney, CD2AP was expressed primarily in glomerular epithelial cells. Knockout mice exhibited defects in epithelial cell foot processes, accompanied by mesangial cell hyperplasia and extracellular matrix deposition. CD2AP associated with nephrin (OMIM Ref. No. 602716), which is the primary component of the slit diaphragm. This observation supports a role for CD2AP in this specialized cell junction.

It is appreciated that the abovementioned animal model for CD2AP is acknowledged by those skilled in the art as a scientifically valid animal model, as can be further appreciated from the publications sited hereinbelow.

Full details of the abovementioned studies are described in the following publications, the disclosure of which are hereby incorporated by reference:
Kirsch, K. H.; Georgescu, M.-M.; Ishimaru, S.; Hanafusa, H.: CMS: an adapter molecule involved in cytoskeletal rearrangements. Proc. Nat. Acad. Sci. 96: 6211-6216, 1999.; and
Shih, N.-Y.; Li, J.; Karpitskii, V.; Nguyen, A.; Dustin, M. L.; Kanagawa, O.; Miner, J. H.; Shaw, A. S.: Congenital nephrotic syndrome in mice lacking CD2-associated protein. Science 2.

Further studies establishing the function and utilities of CD2AP are found in John Hopkins OMIM database record ID 604241, and in sited publications numbered 1198-1200 listed in the bibliography section hereinbelow, which are also hereby incorporated by reference. Hedgehog Interacting Protein (HHIP, Accession NM_{—}022475) is another VGAM87 host target gene. HHIP BINDING SITE is HOST TARGET binding site found in the 5′ untranslated region of mRNA encoded by HHIP, corresponding to a HOST TARGET binding site such as BINDING SITE I, BINDING SITE II or BINDING SITE III. Table 2 illustrates the complementarity of the nucleotide sequences of HHIP BINDING SITE, designated SEQ ID:1989, to the nucleotide sequence of VGAM87 RNA, herein designated VGAM RNA, also designated SEQ ID:422.

Another function of VGAM87 is therefore inhibition of Hedgehog Interacting Protein (HHIP, Accession NM_{—}022475), a gene which is involved in many fundamental processes in embryonic development, including anteroposterior patterns of limbs and regulation of left-right asymmetry. Accordingly, utilities of VGAM87 include diagnosis, prevention and treatment of diseases and clinical conditions associated with HHIP. The function of HHIP has been established by previous studies. Chuang and McMahon (1999) cloned a mouse hedgehog-interacting protein cDNA from a limb bud cDNA expression library. They determined that the Hip cDNA encodes a type I transmembrane glycoprotein, which is expressed in all hedgehog target tissues and binds all 3 mammalian hedgehog proteins (SHH, 600725; IHH, 600726; DHH, 605423) with an affinity similar to that of the Ptc protein (OMIM Ref. No. 601309), which is also a component of the Hh signaling pathway. Like Ptc, Hip expression is up-regulated in response to Hh signaling. Ectopic expression of Hip in transgenic mice results in severe skeletal defects similar to those observed in Indian hedgehog (1HH) mutants, demonstrating that Hip is involved in the attenuation of hedgehog signaling. By database searching with the mouse Hip sequence as query, followed by RT-PCR and RACE analysis using human adult testis cDNA, Bak et al. (2001) cloned a HIP cDNA encoding a deduced 700-amino acid protein that shares 94% sequence identity with mouse Hip. By RT-PCR analysis, HIP expression was detected in all fetal and adult tissues examined except fetal ovary and at very low levels in placenta.

Full details of the abovementioned studies are described in the following publications, the disclosure of which are hereby incorporated by reference:
Bak, M.; Hansen, C.; Henriksen, K. F.; Tommerup, N.: The human hedgehog-interacting protein gene: structure and chromosome mapping to 4q31.21-q31.3. Cytogenet. Cell Genet. 92: 300-303, 2001.; and
Chuang, P.-T.; McMahon, A. P.: Vertebrate hedgehog signalling modulated by induction of a hedgehog-binding protein. Nature 397: 617-621, 1999.

Further studies establishing the function and utilities of HHIP are found in John Hopkins OMIM database record ID 606178, and in sited publications numbered 988-989 listed in the bibliography section hereinbelow, which are also hereby incorporated by reference. A Kinase (PRKA) Anchor Protein 7 (AKAP7, Accession NM_{—}004842) is another VGAM87 host target gene. AKAP7 BINDING SITE1 through AKAP7 BINDING SITE3 are HOST TARGET binding sites found in untranslated regions of mRNA encoded by AKAP7, corresponding to HOST TARGET binding sites such as BINDING SITE I, BINDING SITE II or BINDING SITE III. Table 2 illustrates the complementarity of the nucleotide sequences of AKAP7 BINDING SITE1 through AKAP7 BINDING SITE3, designated SEQ ID:1159, SEQ ID:1683 and SEQ ID:2446 respectively, to the nucleotide sequence of VGAM87 RNA, herein designated VGAM RNA, also designated SEQ ID:422.

Another function of VGAM87 is therefore inhibition of A Kinase (PRKA) Anchor Protein 7 (AKAP7, Accession NM_{—}004842). Accordingly, utilities of VGAM87 include diagnosis, prevention and treatment of diseases and clinical conditions associated with AKAP7. Chromosome 22 Open Reading Frame 19 (C22orf19, Accession NM_{—}003678) is another VGAM87 host target gene. C22orf19 BINDING SITE is HOST TARGET binding site found in the 3′ untranslated region of mRNA encoded by C22orf19, corresponding to a HOST TARGET binding site such as BINDING SITE I, BINDING SITE II or BINDING SITE III. Table 2 illustrates the complementarity of the nucleotide sequences of C22orf19 BINDING SITE, designated SEQ ID: 1048, to the nucleotide sequence of VGAM87 RNA, herein designated VGAM RNA, also designated SEQ ID:422.

Another function of VGAM87 is therefore inhibition of Chromosome 22 Open Reading Frame 19 (C22orf19, Accession NM_{—}003678). Accordingly, utilities of VGAM87 include diagnosis, prevention and treatment of diseases and clinical conditions associated with C22orf19. DKFZp566D234 (Accession XM_{—}030162) is another VGAM87 host target gene. DKFZp566D234 BINDING SITE is HOST TARGET binding site found in the 3′ untranslated region of mRNA encoded by DKFZp566D234, corresponding to a HOST TARGET binding site such as BINDING SITE I, BINDING SITE II or BINDING SITE III. Table 2 illustrates the complementarity of the nucleotide sequences of DKFZp566D234 BINDING SITE, designated SEQ ID:2611, to the nucleotide sequence of VGAM87 RNA, herein designated VGAM RNA, also designated SEQ ID:422.

Another function of VGAM87 is therefore inhibition of DKFZp566D234 (Accession XM_{—}030162). Accordingly, utilities of VGAM87 include diagnosis, prevention and treatment of diseases and clinical conditions associated with DKFZp566D234. FLJ11160 (Accession NM_{—}018344) is another VGAM87 host target gene. FLJ11160 BINDING SITE is HOST TARGET binding site found in the 3′ untranslated region of mRNA encoded by FLJ11160, corresponding to a HOST TARGET binding site such as BINDING SITE I, BINDING SITE II or BINDING SITE III. Table 2 illustrates the complementarity of the nucleotide sequences of FLJ11160 BINDING SITE, designated SEQ ID:1815, to the nucleotide sequence of VGAM87 RNA, herein designated VGAM RNA, also designated SEQ ID:422.

Another function of VGAM87 is therefore inhibition of FLJ11160 (Accession NM_{—}018344). Accordingly, utilities of VGAM87 include diagnosis, prevention and treatment of diseases and clinical conditions associated with FLJ11160. FLJ13089 (Accession XM_{—}048016) is another VGAM87 host target gene. FLJ13089 BINDING SITE is HOST TARGET binding site found in the 5′ untranslated region of mRNA encoded by FLJ13089, corresponding to a HOST TARGET binding site such as BINDING SITE I, BINDING SITE II or BINDING SITE III. Table 2 illustrates the complementarity of the nucleotide sequences of FLJ13089 BINDING SITE, designated SEQ ID:2899, to the nucleotide sequence of VGAM87 RNA, herein designated VGAM RNA, also designated SEQ ID:422.

Another function of VGAM87 is therefore inhibition of FLJ13089 (Accession XM_{—}048016). Accordingly, utilities of VGAM87 include diagnosis, prevention and treatment of diseases and clinical conditions associated with FLJ13089. FLJ14297 (Accession NM_{—}024903) is another VGAM87 host target gene. FLJ14297 BINDING SITE is HOST TARGET binding site found in the 3′ untranslated region of mRNA encoded by FLJ14297, corresponding to a HOST TARGET binding site such as BINDING SITE I, BINDING SITE II or BINDING SITE III. Table 2 illustrates the complementarity of the nucleotide sequences of FLJ14297 BINDING SITE, designated SEQ ID:2107, to the nucleotide sequence of VGAM87 RNA, herein designated VGAM RNA, also designated SEQ ID:422.

Another function of VGAM87 is therefore inhibition of FLJ14297 (Accession NM_{—}024903). Accordingly, utilities of VGAM87 include diagnosis, prevention and treatment of diseases and clinical conditions associated with FLJ14297. HSPC065 (Accession NM_{—}014157) is another VGAM87 host target gene. HSPC065 BINDING SITE is HOST TARGET binding site found in the 3′ untranslated region of mRNA encoded by HSPC065, corresponding to a HOST TARGET binding site such as BINDING SITE I, BINDING SITE II or BINDING SITE III. Table 2 illustrates the complementarity of the nucleotide sequences of HSPC065 BINDING SITE, designated SEQ ID: 1483, to the nucleotide sequence of VGAM87 RNA, herein designated VGAM RNA, also designated SEQ ID:422.

Another function of VGAM87 is therefore inhibition of HSPC065 (Accession NM_{—}014157). Accordingly, utilities of VGAM87 include diagnosis, prevention and treatment of diseases and clinical conditions associated with HSPC065. LOC150737 (Accession XM_{—}086984) is another VGAM87 host target gene. LOC150737 BINDING SITE is HOST TARGET binding site found in the 3′ untranslated region of mRNA encoded by LOC150737, corresponding to a HOST TARGET binding site such as BINDING SITE I, BINDING SITE II or BINDING SITE III. Table 2 illustrates the complementarity of the nucleotide sequences of LOC150737 BINDING SITE, designated SEQ ID:3149, to the nucleotide sequence of VGAM87 RNA, herein designated VGAM RNA, also designated SEQ ID:422.

Another function of VGAM87 is therefore inhibition of LOC150737 (Accession XM_{—}086984). Accordingly, utilities of VGAM87 include diagnosis, prevention and treatment of diseases and clinical conditions associated with LOC150737. LOC152185 (Accession NM_{—}144718) is another VGAM87 host target gene. LOC152185 BINDING SITE is HOST TARGET binding site found in the 3′ untranslated region of mRNA encoded by LOC152185, corresponding to a HOST TARGET binding site such as BINDING SITE I, BINDING SITE II or BINDING SITE III. Table 2 illustrates the complementarity of the nucleotide sequences of LOC152185 BINDING SITE, designated SEQ ID:2498, to the nucleotide sequence of VGAM87 RNA, herein designated VGAM RNA, also designated SEQ ID:422.

Another function of VGAM87 is therefore inhibition of LOC152185 (Accession NM_{—}144718). Accordingly, utilities of VGAM87 include diagnosis, prevention and treatment of diseases and clinical conditions associated with LOC152185. FIG. 1 further provides a conceptual description of a novel bioinformatically detected viral gene of the present invention, referred to here as Viral Genomic Address Messenger 88 (VGAM88) viral gene, which modulates expression of respective host target genes thereof, the function and utility of which host target genes is known in the art.

VGAM88 is a novel bioinformatically detected regulatory, non protein coding, viral micro RNA (miRNA) gene. The method by which VGAM88 was detected is described hereinabove with reference to FIGS. 1-8.

VGAM88 gene, herein designated VGAM GENE, is a viral gene contained in the genome of Vaccinia Virus. VGAM88 host target gene, herein designated VGAM HOST TARGET GENE, is a human gene contained in the human genome.

VGAM88 gene encodes a VGAM88 precursor RNA, herein designated VGAM PRECURSOR RNA. Similar to other miRNA genes, and unlike most ordinary genes, VGAM88 precursor RNA does not encode a protein. A nucleotide sequence identical or highly similar to the nucleotide sequence of VGAM88 precursor RNA is designated SEQ ID:74, and is provided hereinbelow with reference to the sequence listing part. Nucleotide sequence SEQ ID:74 is located at position 29293 relative to the genome of Vaccinia Virus.

VGAM88 precursor RNA folds onto itself, forming VGAM88 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM88 folded precursor RNA into VGAM88 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM88 RNA is designated SEQ ID:423, and is provided hereinbelow with reference to the sequence listing part.

VGAM88 host target gene, herein designated VGAM HOST TARGET GENE, encodes a corresponding messenger RNA, VGAM88 host target RNA, herein designated VGAM HOST TARGET RNA. VGAM88 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM88 RNA, herein designated VGAM RNA, binds complementarily to one or more host target binding sites located in untranslated regions of VGAM88 host target RNA, herein designated VGAM HOST TARGET RNA. This complementary binding is due to the fact that the nucleotide sequence of VGAM88 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM88 RNA, herein designated VGAM RNA, may have a different number of host target binding sites in untranslated regions of a VGAM88 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM88 RNA, herein designated VGAM RNA, to host target binding sites on VGAM88 host target RNA, herein designated VGAM HOST TARGET RNA, such as BINDING SITE I, BINDING SITE II and BINDING SITE III, inhibits translation of VGAM88 host target RNA into VGAM88 host target protein, herein designated VGAM HOST TARGET PROTEIN. VGAM host target protein is therefore outlined by a broken line.

It is appreciated that VGAM88 host target gene, herein designated VGAM HOST TARGET GENE, in fact represents a plurality of VGAM88 host target genes. The mRNA of each one of this plurality of VGAM88 host target genes comprises one or more host target binding sites, each having a nucleotide sequence which is at least partly complementary to VGAM88 RNA, herein designated VGAM RNA, and which when bound by VGAM88 RNA causes inhibition of translation of respective one or more VGAM88 host target proteins.

It is further appreciated by one skilled in the art that the mode of translational inhibition illustrated by FIG. 1 with specific reference to translational inhibition exerted by VGAM88 gene, herein designated VGAM GENE, on one or more VGAM88 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM88 is inhibition of expression of host target genes, as part of a novel viral mechanism of attacking a host. Accordingly, utilities of VGAM88 include diagnosis, prevention and treatment of viral infection by Vaccinia Virus. Specific functions, and accordingly utilities, of VGAM88 correlate with, and may be deduced from, the identity of the host target genes which VGAM88 binds and inhibits, and the function of these host target genes, as elaborated hereinbelow.

Nucleotide sequences of the VGAM88 precursor RNA, herein designated VGAM PRECURSOR RNA, and of the ‘diced’ VGAM88 RNA, herein designated VGAM RNA, and a schematic representation of the secondary folding of VGAM88 folded precursor RNA, herein designated VGAM FOLDED PRECURSOR RNA, of VGAM88 are further described hereinbelow with reference to Table 1.

Nucleotide sequences of host target binding sites, such as BINDING SITE-I, BINDING SITE-II and BINDING SITE-III of FIG. 1, found on VGAM88 host target RNA, and schematic representation of the complementarity of each of these host target binding sites to VGAM88 RNA, herein designated VGAM RNA, are described hereinbelow with reference to Table 2.

As mentioned hereinabove with reference to FIG. 1, a function of VGAM88 gene, herein designated VGAM is inhibition of expression of VGAM88 target genes. It is appreciated that specific functions, and accordingly utilities, of VGAM88 correlate with, and may be deduced from, the identity of the target genes which VGAM88 binds and inhibits, and the function of these target genes, as elaborated hereinbelow.

Dishevelled Associated Activator of Morphogenesis 2 (DAAM2, Accession XM_{—}166434) is a VGAM88 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3560, to the nucleotide sequence of VGAM88 RNA, herein designated VGAM RNA, also designated SEQ ID:423.

A function of VGAM88 is therefore inhibition of Dishevelled Associated Activator of Morphogenesis 2 (DAAM2, Accession XM_{—}166434), a gene which controls cell polarity and movement during development. Accordingly, utilities of VGAM88 include diagnosis, prevention and treatment of diseases and clinical conditions associated with DAAM2. The function of DAAM2 and its association with various diseases and clinical conditions, has been established by previous studies, as described hereinabove with reference to VGAM15. FN14 (Accession NM_{—}016639) is another VGAM88 host target gene. FN14 BINDING SITE is HOST TARGET binding site found in the 3′ untranslated region of mRNA encoded by FN14, corresponding to a HOST TARGET binding site such as BINDING SITE I, BINDING SITE II or BINDING SITE III. Table 2 illustrates the complementarity of the nucleotide sequences of FN14 BINDING SITE, designated SEQ ID:1703, to the nucleotide sequence of VGAM88 RNA, herein designated VGAM RNA, also designated SEQ ID:423.

Another function of VGAM88 is therefore inhibition of FN14 (Accession NM_{—}016639), a gene which may be a transmembrane protein that reduces plasma membrane associations with vitronectin and fibronectin. Accordingly, utilities of VGAM88 include diagnosis, prevention and treatment of diseases and clinical conditions associated with FN14. The function of FN14 has been established by previous studies. Using a differential display approach to isolate cDNA fragments representing fibroblast growth factor-1 (FGF1; 131220)-inducible genes, Meighan-Mantha et al. (1999) isolated a mouse cDNA encoding Fn14, which they characterized as an immediate-early response gene. By searching an EST database with the mouse Fn14 sequence, Feng et al. (2000) identified cDNAs encoding human FN14. The predicted 129-amino acid FN14 protein, which shares 82% amino acid identity with the mouse sequence, contains a signal peptide, an extracellular domain, a membrane-anchoring domain, and a cytoplasmic domain. Northern blot analysis detected increased FN14 expression in response to FGF1, calf serum, or phorbol ester stimulation of human quiescent fibroblasts in vitro. A 1.2-kb FN14 transcript was expressed at high levels in heart, placenta, and kidney, at intermediate levels in lung, skeletal muscle, and pancreas, and at low levels in brain and liver. In addition, elevated FN14 expression was found in human liver cancer cell lines and hepatocellular carcinoma specimens. Expression of mouse Fn14 was up-regulated in hepatocellular carcinoma nodules that develop in 2 different transgenic mouse models of hepatocarcino-genesis. Rapid induction of Fn14 expression occurred during mouse liver regeneration after partial hepatectomy. Feng et al. (2000) concluded that FN14 may play a role in hepatocyte growth control and liver neoplasia. Using expression cloning and panning of an endothelial cell cDNA library with the C-terminal receptor-binding domain of TWEAK (TNFSF12; 602695) as the probe, followed by slide binding analysis, Wiley et al. (2001) isolated a cDNA encoding FN14, which they termed TWEAKR. Sequence analysis predicted that TWEAKR has a single extracellular cysteine-rich region that is homologous to those observed in TNFRSF1A (OMIM Ref. No. 191190) and some other TNFR family members. TWEAKR also has a cytoplasmic TRAF (see OMIM Ref. No. TRAF2; 601895)-binding site. Different binding analyses indicated a physiologically relevant affinity between TWEAK and TWEAKR. GST-binding analysis showed an interaction of the TWEAKR cytoplasmic region with TRAF1 (OMIM Ref. No. 601711), TRAF2, and, to a lesser extent, TRAF3 (OMIM Ref. No. 601896), but not with other TRAFs tested. Northern blot analysis revealed expression of a 1.2-kb Tweakr transcript in rat aortic smooth muscle cells. Tweakr expression could be up-regulated by a number of growth factors. Blocking of TWEAKR signaling inhibited the migration of renal microvascular cells in vitro, indicating that endogenous TWEAK regulates endothelial cell wound closure rates. Wiley et al. (2001) concluded that TWEAKR is a fully functional receptor for TWEAK and that the TWEAK-TWEAKR system plays a role in endothelial cell growth and migration.

Full details of the abovementioned studies are described in the following publications, the disclosure of which are hereby incorporated by reference:
Feng, S.-L. Y.; Guo, Y.; Factor, V. M.; Thorgeirsson, S. S.; Bell, D. W.; Testa, J. R.; Peifley, K. A.; Winkles, J. A.: The Fn14 immediate-early response gene is induced during liver regeneration and highly expressed in both human and murine hepatocellular carcinomas. Am. J. Path. 156: 1253-1261, 2000.; and
Wiley, S. R.; Cassiano, L.; Lofton, T.; Davis-Smith, T.; Winkles, J. A.; Lindner, V.; Liu, H.; Daniel, T. O.; Smith, C. A.; Fanslow, W. C.: A novel TNF receptor family member binds TW.

Further studies establishing the function and utilities of FN14 are found in John Hopkins OMIM database record ID 605914, and in sited publications numbered 181-18 and 1822 listed in the bibliography section hereinbelow, which are also hereby incorporated by reference. Transmembrane 4 Super family Member 2 (TM4SF2, Accession NM_{—}004615) is another VGAM88 host target gene. TM4SF2 BINDING SITE is HOST TARGET binding site found in the 3′ untranslated region of mRNA encoded by TM4SF2, corresponding to a HOST TARGET binding site such as BINDING SITE I, BINDING SITE II or BINDING SITE III. Table 2 illustrates the complementarity of the nucleotide sequences of TM4SF2 BINDING SITE, designated SEQ ID:1131, to the nucleotide sequence of VGAM88 RNA, herein designated VGAM RNA, also designated SEQ ID:423.

Another function of VGAM88 is therefore inhibition of Transmembrane 4 Super family Member 2 (TM4SF2, Accession NM_{—}004615). Accordingly, utilities of VGAM88 include diagnosis, prevention and treatment of diseases and clinical conditions associated with TM4SF2. Wingless-type MMTV Integration Site Family, Member 3 (WNT3, Accession NM_{—}030753) is another VGAM88 host target gene. WNT3 BINDING SITE is HOST TARGET binding site found in the 3′ untranslated region of mRNA encoded by WNT3, corresponding to a HOST TARGET binding site such as BINDING SITE I, BINDING SITE II or BINDING SITE III. Table 2 illustrates the complementarity of the nucleotide sequences of WNT3 BINDING SITE, designated SEQ ID:2159, to the nucleotide sequence of VGAM88 RNA, herein designated VGAM RNA, also designated SEQ ID:423.

Another function of VGAM88 is therefore inhibition of Wingless-type MMTV Integration Site Family, Member 3 (WNT3, Accession NM_{—}030753), a gene which is the ligand for members of the frizzled family of seven transmembrane receptors and has roles in cell-cell signaling. Accordingly, utilities of VGAM88 include diagnosis, prevention and treatment of diseases and clinical conditions associated with WNT3. The function of WNT3 has been established by previous studies. Rider et al. (1989) assigned the INT4 gene to 17q21-q22 using a DNA probe in the study of a panel of chromosome-mediated gene transfectants and conventional hybrids, in particular those with well-defined breaks on human chromosome 17. In situ hybridization was performed for more precise localization. The mouse MMTV integration site int-4 was mapped to mouse chromosome 11 in a region homologous to the region of human chromosome 17 carrying the INT4 locus. For the group of related genes of which the first to be discovered was INT1 (OMIM Ref. No. 164820), Nusse et al. (1991) suggested the designation Wnt (pronounced ‘wint’), a mnemonic for the ‘wingless’ homolog. The product INT1 (renamed WNT1) encodes a novel secretory glycoprotein similar to the product of the Drosophila melanogaster ‘wingless’ gene. The INT4 locus was renamed WNT3. Roelink et al. (1993) used mouse Wnt3 sequences as a probe to isolate a genomic clone of the human homolog, WNT3. Comparison of the deduced mouse and human WNT3 protein sequences showed 4 changes in 333 amino acids. The gene was localized to 17q21 by isotopic in situ hybridization. Several studies had implicated Wnt signaling in primary axis formation during vertebrate embryogenesis, yet no Wnt protein had been shown to be essential for this process. In the mouse, primitive streak formation is the first overt morphologic sign of the anterior-posterior axis in mesoderm. Liu et al. (1999) generated Wnt3 −/− mice by targeted disruption of the mouse Wnt3 gene. Wnt3 −/− mice developed a normal egg cylinder but did not form a primitive streak, mesoderm, or node. The epiblast continued to proliferate in an undifferentiated state that lacked anterior-posterior neural patterning, but anterior visceral endoderm markers were expressed and correctly positioned. Liu et al. (1999) concluded that regional patterning of the visceral endoderm is independent of primitive streak formation, but the subsequent establishment of anterior-posterior neural pattern in the ectoderm is dependent on derivatives of the primitive streak. Their studies provided genetic proof for the requirement of Wnt3 in primary axis formation in the mouse. Using ribonuclease protection analysis, Huguet et al. (1994) investigated expression of WNT genes, including WNT3, in human cell lines, as well as in normal, benign, and malignant breast tissue. They detected WNT3 in breast cell lines and in breast tissue.

Full details of the abovementioned studies are described in the following publications, the disclosure of which are hereby incorporated by reference:
Nusse, R.; Brown, A.; Papkoff, J.; Scambler, P.; Shackleford, G.; McMahon, A.; Moon, R.; Varmus, H.: A new nomenclature for int-1 and related genes: the Wnt gene family. Cell 64: 231-232, 1991.; and
Rider, S. H.; Gorman, P. A.; Shipley, J.; Roeling, H.; Nusse, R.; Xu, W.; Sheer, D.; Solomon, E.: Localisation of the human int-4 (INT4) gene. (Abstract) Cytogenet. Cell Genet. 51: 1066.

Further studies establishing the function and utilities of WNT3 are found in John Hopkins OMIM database record ID 165330, and in sited publications numbered 895, 241 and 2418-2419 listed in the bibliography section hereinbelow, which are also hereby incorporated by reference. GLTP (Accession NM_{—}016433) is another VGAM88 host target gene. GLTP BINDING SITE is HOST TARGET binding site found in the 3′ untranslated region of mRNA encoded by GLTP, corresponding to a HOST TARGET binding site such as BINDING SITE I, BINDING SITE II or BINDING SITE III. Table 2 illustrates the complementarity of the nucleotide sequences of GLTP BINDING SITE, designated SEQ ID: 1686, to the nucleotide sequence of VGAM88 RNA, herein designated VGAM RNA, also designated SEQ ID:423.

Another function of VGAM88 is therefore inhibition of GLTP (Accession NM_{—}016433). Accordingly, utilities of VGAM88 include diagnosis, prevention and treatment of diseases and clinical conditions associated with GLTP. PRO0149 (Accession NM_{—}014117) is another VGAM88 host target gene. PRO0149 BINDING SITE is HOST TARGET binding site found in the 5′ untranslated region of mRNA encoded by PRO0149, corresponding to a HOST TARGET binding site such as BINDING SITE I, BINDING SITE II or BINDING SITE III. Table 2 illustrates the complementarity of the nucleotide sequences of PRO0149 BINDING SITE, designated SEQ ID: 1473, to the nucleotide sequence of VGAM88 RNA, herein designated VGAM RNA, also designated SEQ ID:423.

Another function of VGAM88 is therefore inhibition of PRO0149 (Accession NM_{—}014117). Accordingly, utilities of VGAM88 include diagnosis, prevention and treatment of diseases and clinical conditions associated with PRO0149. FIG. 1 further provides a conceptual description of a novel bioinformatically detected viral gene of the present invention, referred to here as Viral Genomic Address Messenger 89 (VGAM89) viral gene, which modulates expression of respective host target genes thereof, the function and utility of which host target genes is known in the art.

VGAM89 is a novel bioinformatically detected regulatory, non protein coding, viral micro RNA (miRNA) gene. The method by which VGAM89 was detected is described hereinabove with reference to FIGS. 1-8.

VGAM89 gene, herein designated VGAM GENE, is a viral gene contained in the genome of Vaccinia Virus. VGAM89 host target gene, herein designated VGAM HOST TARGET GENE, is a human gene contained in the human genome.

VGAM89 gene encodes a VGAM89 precursor RNA, herein designated VGAM PRECURSOR RNA. Similar to other miRNA genes, and unlike most ordinary genes, VGAM89 precursor RNA does not encode a protein. A nucleotide sequence identical or highly similar to the nucleotide sequence of VGAM89 precursor RNA is designated SEQ ID:75, and is provided hereinbelow with reference to the sequence listing part. Nucleotide sequence SEQ ID:75 is located at position 25248 relative to the genome of Vaccinia Virus.

VGAM89 precursor RNA folds onto itself, forming VGAM89 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM89 folded precursor RNA into VGAM89 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM89 RNA is designated SEQ ID:424, and is provided hereinbelow with reference to the sequence listing part.

VGAM89 host target gene, herein designated VGAM HOST TARGET GENE, encodes a corresponding messenger RNA, VGAM89 host target RNA, herein designated VGAM HOST TARGET RNA. VGAM89 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM89 RNA, herein designated VGAM RNA, binds complementarily to one or more host target binding sites located in untranslated regions of VGAM89 host target RNA, herein designated VGAM HOST TARGET RNA. This complementary binding is due to the fact that the nucleotide sequence of VGAM89 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM89 RNA, herein designated VGAM RNA, may have a different number of host target binding sites in untranslated regions of a VGAM89 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM89 RNA, herein designated VGAM RNA, to host target binding sites on VGAM89 host target RNA, herein designated VGAM HOST TARGET RNA, such as BINDING SITE I, BINDING SITE II and BINDING SITE III, inhibits translation of VGAM89 host target RNA into VGAM89 host target protein, herein designated VGAM HOST TARGET PROTEIN. VGAM host target protein is therefore outlined by a broken line.

It is appreciated that VGAM89 host target gene, herein designated VGAM HOST TARGET GENE, in fact represents a plurality of VGAM89 host target genes. The mRNA of each one of this plurality of VGAM89 host target genes comprises one or more host target binding sites, each having a nucleotide sequence which is at least partly complementary to VGAM89 RNA, herein designated VGAM RNA, and which when bound by VGAM89 RNA causes inhibition of translation of respective one or more VGAM89 host target proteins.

It is further appreciated by one skilled in the art that the mode of translational inhibition illustrated by FIG. 1 with specific reference to translational inhibition exerted by VGAM89 gene, herein designated VGAM GENE, on one or more VGAM89 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM89 is inhibition of expression of host target genes, as part of a novel viral mechanism of attacking a host. Accordingly, utilities of VGAM89 include diagnosis, prevention and treatment of viral infection by Vaccinia Virus. Specific functions, and accordingly utilities, of VGAM89 correlate with, and may be deduced from, the identity of the host target genes which VGAM89 binds and inhibits, and the function of these host target genes, as elaborated hereinbelow.

Nucleotide sequences of the VGAM89 precursor RNA, herein designated VGAM PRECURSOR RNA, and of the ‘diced’ VGAM89 RNA, herein designated VGAM RNA, and a schematic representation of the secondary folding of VGAM89 folded precursor RNA, herein designated VGAM FOLDED PRECURSOR RNA, of VGAM89 are further described hereinbelow with reference to Table 1.

Nucleotide sequences of host target binding sites, such as BINDING SITE-I, BINDING SITE-II and BINDING SITE-III of FIG. 1, found on VGAM89 host target RNA, and schematic representation of the complementarity of each of these host target binding sites to VGAM89 RNA, herein designated VGAM RNA, are described hereinbelow with reference to Table 2.

As mentioned hereinabove with reference to FIG. 1, a function of VGAM89 gene, herein designated VGAM is inhibition of expression of VGAM89 target genes. It is appreciated that specific functions, and accordingly utilities, of VGAM89 correlate with, and may be deduced from, the identity of the target genes which VGAM89 binds and inhibits, and the function of these target genes, as elaborated hereinbelow.

Lysosomal-associated Membrane Protein 2 (LAMP2, Accession NM_{—}013995) is a VGAM89 host target gene. LAMP2 BINDING SITE is HOST TARGET binding site found in the 3′ untranslated region of mRNA encoded by LAMP2, corresponding to a HOST TARGET binding site such as BINDING SITE I, BINDING SITE II or BINDING SITE III. Table 2 illustrates the complementarity of the nucleotide sequences of LAMP2 BINDING SITE, designated SEQ ID:1455, to the nucleotide sequence of VGAM89 RNA, herein designated VGAM RNA, also designated SEQ ID:424.

A function of VGAM89 is therefore inhibition of Lysosomal-associated Membrane Protein 2 (LAMP2, Accession NM_{—}013995). Accordingly, utilities of VGAM89 include diagnosis, prevention and treatment of diseases and clinical conditions associated with LAMP2. DKFZp762K2015 (Accession XM_{—}051791) is another VGAM89 host target gene. DKFZp762K2015 BINDING SITE is HOST TARGET binding site found in the 3′ untranslated region of mRNA encoded by DKFZp762K2015, corresponding to a HOST TARGET binding site such as BINDING SITE I, BINDING SITE II or BINDING SITE III. Table 2 illustrates the complementarity of the nucleotide sequences of DKFZp762K2015 BINDING SITE, designated SEQ ID:2949, to the nucleotide sequence of VGAM89 RNA, herein designated VGAM RNA, also designated SEQ ID:424.

Another function of VGAM89 is therefore inhibition of DKFZp762K2015 (Accession XM_{—}051791). Accordingly, utilities of VGAM89 include diagnosis, prevention and treatment of diseases and clinical conditions associated with DKFZp762K2015. FLJ10921 (Accession NM_{—}018272) is another VGAM89 host target gene. FLJ10921 BINDING SITE is HOST TARGET binding site found in the 5′ untranslated region of mRNA encoded by FLJ10921, corresponding to a HOST TARGET binding site such as BINDING SITE I, BINDING SITE II or BINDING SITE III. Table 2 illustrates the complementarity of the nucleotide sequences of FLJ10921 BINDING SITE, designated SEQ ID:1808, to the nucleotide sequence of VGAM89 RNA, herein designated VGAM RNA, also designated SEQ ID:424.

Another function of VGAM89 is therefore inhibition of FLJ10921 (Accession NM_{—}018272). Accordingly, utilities of VGAM89 include diagnosis, prevention and treatment of diseases and clinical conditions associated with FLJ10921. LOC91752 (Accession XM_{—}040403) is another VGAM89 host target gene. LOC91752 BINDING SITE is HOST TARGET binding site found in the 3′ untranslated region of mRNA encoded by LOC91752, corresponding to a HOST TARGET binding site such as BINDING SITE I, BINDING SITE II or BINDING SITE III. Table 2 illustrates the complementarity of the nucleotide sequences of LOC91752 BINDING SITE, designated SEQ ID:2778, to the nucleotide sequence of VGAM89 RNA, herein designated VGAM RNA, also designated SEQ ID:424.

Another function of VGAM89 is therefore inhibition of LOC91752 (Accession XM_{—}040403). Accordingly, utilities of VGAM89 include diagnosis, prevention and treatment of diseases and clinical conditions associated with LOC91752. FIG. 1 further provides a conceptual description of a novel bioinformatically detected viral gene of the present invention, referred to here as Viral Genomic Address Messenger 90 (VGAM90) viral gene, which modulates expression of respective host target genes thereof, the function and utility of which host target genes is known in the art.

VGAM90 is a novel bioinformatically detected regulatory, non protein coding, viral micro RNA (miRNA) gene. The method by which VGAM90 was detected is described hereinabove with reference to FIGS. 1-8.

VGAM90 gene, herein designated VGAM GENE, is a viral gene contained in the genome of Vaccinia Virus. VGAM90 host target gene, herein designated VGAM HOST TARGET GENE, is a human gene contained in the human genome.

VGAM90 gene encodes a VGAM90 precursor RNA, herein designated VGAM PRECURSOR RNA. Similar to other miRNA genes, and unlike most ordinary genes, VGAM90 precursor RNA does not encode a protein. A nucleotide sequence identical or highly similar to the nucleotide sequence of VGAM90 precursor RNA is designated SEQ ID:76, and is provided hereinbelow with reference to the sequence listing part. Nucleotide sequence SEQ ID:76 is located at position 29027 relative to the genome of Vaccinia Virus.

VGAM90 precursor RNA folds onto itself, forming VGAM90 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM90 folded precursor RNA into VGAM90 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM90 RNA is designated SEQ ID:425, and is provided hereinbelow with reference to the sequence listing part.

VGAM90 host target gene, herein designated VGAM HOST TARGET GENE, encodes a corresponding messenger RNA, VGAM90 host target RNA, herein designated VGAM HOST TARGET RNA. VGAM90 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM90 RNA, herein designated VGAM RNA, binds complementarily to one or more host target binding sites located in untranslated regions of VGAM90 host target RNA, herein designated VGAM HOST TARGET RNA. This complementary binding is due to the fact that the nucleotide sequence of VGAM90 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM90 RNA, herein designated VGAM RNA, may have a different number of host target binding sites in untranslated regions of a VGAM90 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM90 RNA, herein designated VGAM RNA, to host target binding sites on VGAM90 host target RNA, herein designated VGAM HOST TARGET RNA, such as BINDING SITE I, BINDING SITE II and BINDING SITE III, inhibits translation of VGAM90 host target RNA into VGAM90 host target protein, herein designated VGAM HOST TARGET PROTEIN. VGAM host target protein is therefore outlined by a broken line.

It is appreciated that VGAM90 host target gene, herein designated VGAM HOST TARGET GENE, in fact represents a plurality of VGAM90 host target genes. The mRNA of each one of this plurality of VGAM90 host target genes comprises one or more host target binding sites, each having a nucleotide sequence which is at least partly complementary to VGAM90 RNA, herein designated VGAM RNA, and which when bound by VGAM90 RNA causes inhibition of translation of respective one or more VGAM90 host target proteins.

It is further appreciated by one skilled in the art that the mode of translational inhibition illustrated by FIG. 1 with specific reference to translational inhibition exerted by VGAM90 gene, herein designated VGAM GENE, on one or more VGAM90 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM90 is inhibition of expression of host target genes, as part of a novel viral mechanism of attacking a host. Accordingly, utilities of VGAM90 include diagnosis, prevention and treatment of viral infection by Vaccinia Virus. Specific functions, and accordingly utilities, of VGAM90 correlate with, and may be deduced from, the identity of the host target genes which VGAM90 binds and inhibits, and the function of these host target genes, as elaborated hereinbelow.

Nucleotide sequences of the VGAM90 precursor RNA, herein designated VGAM PRECURSOR RNA, and of the ‘diced’ VGAM90 RNA, herein designated VGAM RNA, and a schematic representation of the secondary folding of VGAM90 folded precursor RNA, herein designated VGAM FOLDED PRECURSOR RNA, of VGAM90 are further described hereinbelow with reference to Table 1.

Nucleotide sequences of host target binding sites, such as BINDING SITE-I, BINDING SITE-II and BINDING SITE-III of FIG. 1, found on VGAM90 host target RNA, and schematic representation of the complementarity of each of these host target binding sites to VGAM90 RNA, herein designated VGAM RNA, are described hereinbelow with reference to Table 2.

As mentioned hereinabove with reference to FIG. 1, a function of VGAM90 gene, herein designated VGAM is inhibition of expression of VGAM90 target genes. It is appreciated that specific functions, and accordingly utilities, of VGAM90 correlate with, and may be deduced from, the identity of the target genes which VGAM90 binds and inhibits, and the function of these target genes, as elaborated hereinbelow.

Glucagon-like Peptide 1 Receptor (GLP1R, Accession NM_{—}002062) is a VGAM90 host target gene. GLP1R BINDING SITE is HOST TARGET binding site found in the 3′ untranslated region of mRNA encoded by GLP1R, corresponding to a HOST TARGET binding site such as BINDING SITE I, BINDING SITE II or BINDING SITE III. Table 2 illustrates the complementarity of the nucleotide sequences of GLP1R BINDING SITE, designated SEQ ID:896, to the nucleotide sequence of VGAM90 RNA, herein designated VGAM RNA, also designated SEQ ID:425.

A function of VGAM90 is therefore inhibition of Glucagon-like Peptide 1 Receptor (GLP1R, Accession NM_{—}002062), a gene which is mediated by g proteins which activate adenylyl cyclase. Accordingly, utilities of VGAM90 include diagnosis, prevention and treatment of diseases and clinical conditions associated with GLP1R. The function of GLP1R has been established by previous studies. Glucagon-like peptide-1 (GLP1) is a hormone derived from the preproglucagon molecule (OMIM Ref. No. 138030) and is secreted by intestinal L cells. It is the most potent stimulator of glucose-induced insulin secretion and also suppresses in vivo acid secretion by gastric glands. By transient expression of a rat pancreatic islet cDNA library in COS cells, Thorens (1992) isolated a cDNA for the GLP1 receptor (GLP1R). Transfected into COS cells, the receptor bound GLP1 with high affinity and was coupled to activation of adenylate cyclase. It did not bind peptides of related structure and similar function, such as glucagon (GCG; 138030), gastric inhibitory polypeptide (GIP; 137240), vasoactive intestinal peptide (VIP; 192320), or secretin (SCT; 182099). The receptor is 463 amino acids long and contains 7 transmembrane domains. Sequence homology was found only with the receptors for secretin (SCTR; 182098), calcitonin (CALCR; 114131), and parathyroid hormone (PTHR; 168468), which together form a newly characterized family of G-coupled receptors. Dillon et al. (1993) also cloned a cDNA corresponding to the GLP1R gene. By promoter analysis and electrophoretic mobility shift analysis, Wildhage et al. (1999) showed that the GLP1R promoter binds both SP1 (OMIM Ref. No. 189906) and SP3 (OMIM Ref. No. 601804). They concluded that the basal activity of the GLP1R gene is mediated by 2 proximal SP1-binding sites and that a more distal site acts as a repressor.

Full details of the abovementioned studies are described in the following publications, the disclosure of which are hereby incorporated by reference:
Dillon, J. S.; Tanizawa, Y.; Wheeler, M. B.; Leng, X.-H.; Ligon, B. B.; Rabin, D. U.; Yoo-Warren, H.; Permutt, M. A.; Boyd, A. E., III: Cloning and functional expression of the human glucagon-like peptide-1 (GLP-1) receptor. Endocrinology 133: 1907-1910, 1993.; and
Wildhage, I.; Trusheim, H.; Goke, B.; Lankat-Buttgereit, B.: Gene expression of the human glucagon-like peptide-1 receptor is regulated by Sp1 and Sp3. Endocrinology 140: 624-631, 1999.

Further studies establishing the function and utilities of GLP1R are found in John Hopkins OMIM database record ID 138032, and in sited publications numbered 717-72 and 775 listed in the bibliography section hereinbelow, which are also hereby incorporated by reference. FLJ10891 (Accession NM_{—}018260) is another VGAM90 host target gene. FLJ10891 BINDING SITE is HOST TARGET binding site found in the 3′ untranslated region of mRNA encoded by FLJ10891, corresponding to a HOST TARGET binding site such as BINDING SITE I, BINDING SITE II or BINDING SITE III. Table 2 illustrates the complementarity of the nucleotide sequences of FLJ10891 BINDING SITE, designated SEQ ID: 1806, to the nucleotide sequence of VGAM90 RNA, herein designated VGAM RNA, also designated SEQ ID:425.

Another function of VGAM90 is therefore inhibition of FLJ10891 (Accession NM_{—}018260). Accordingly, utilities of VGAM90 include diagnosis, prevention and treatment of diseases and clinical conditions associated with FLJ10891. KIAA0781 (Accession XM_{—}041314) is another VGAM90 host target gene. KIAA0781 BINDING SITE is HOST TARGET binding site found in the 3′ untranslated region of mRNA encoded by KIAA0781, corresponding to a HOST TARGET binding site such as BINDING SITE I, BINDING SITE II or BINDING SITE III. Table 2 illustrates the complementarity of the nucleotide sequences of KIAA0781 BINDING SITE, designated SEQ ID:2793, to the nucleotide sequence of VGAM90 RNA, herein designated VGAM RNA, also designated SEQ ID:425.

Another function of VGAM90 is therefore inhibition of KIAA0781 (Accession XM_{—}041314). Accordingly, utilities of VGAM90 include diagnosis, prevention and treatment of diseases and clinical conditions associated with KIAA0781. KIAA1077 (Accession XM_{—}053496) is another VGAM90 host target gene. KIAA1077 BINDING SITE is HOST TARGET binding site found in the 3′ untranslated region of mRNA encoded by KIAA1077, corresponding to a HOST TARGET binding site such as BINDING SITE I, BINDING SITE II or BINDING SITE III. Table 2 illustrates the complementarity of the nucleotide sequences of KIAA1077 BINDING SITE, designated SEQ ID:2963, to the nucleotide sequence of VGAM90 RNA, herein designated VGAM RNA, also designated SEQ ID:425.

Another function of VGAM90 is therefore inhibition of KIAA1077 (Accession XM_{—}053496). Accordingly, utilities of VGAM90 include diagnosis, prevention and treatment of diseases and clinical conditions associated with KIAA1077. Pellino Homolog 2 (Drosophila) (PELI2, Accession NM_{—}021255) is another VGAM90 host target gene. PELI2 BINDING SITE is HOST TARGET binding site found in the 3′ untranslated region of mRNA encoded by PELI2, corresponding to a HOST TARGET binding site such as BINDING SITE I, BINDING SITE II or BINDING SITE III. Table 2 illustrates the complementarity of the nucleotide sequences of PELI2 BINDING SITE, designated SEQ ID:1950, to the nucleotide sequence of VGAM90 RNA, herein designated VGAM RNA, also designated SEQ ID:425.

Another function of VGAM90 is therefore inhibition of Pellino Homolog 2 (Drosophila) (PELI2, Accession NM_{—}021255). Accordingly, utilities of VGAM90 include diagnosis, prevention and treatment of diseases and clinical conditions associated with PELI2. FIG. 1 further provides a conceptual description of a novel bioinformatically detected viral gene of the present invention, referred to here as Viral Genomic Address Messenger 91 (VGAM91) viral gene, which modulates expression of respective host target genes thereof, the function and utility of which host target genes is known in the art.

VGAM91 is a novel bioinformatically detected regulatory, non protein coding, viral micro RNA (miRNA) gene. The method by which VGAM91 was detected is described hereinabove with reference to FIGS. 1-8.

VGAM91 gene, herein designated VGAM GENE, is a viral gene contained in the genome of Vaccinia Virus. VGAM91 host target gene, herein designated VGAM HOST TARGET GENE, is a human gene contained in the human genome.

VGAM91 gene encodes a VGAM91 precursor RNA, herein designated VGAM PRECURSOR RNA. Similar to other miRNA genes, and unlike most ordinary genes, VGAM91 precursor RNA does not encode a protein. A nucleotide sequence identical or highly similar to the nucleotide sequence of VGAM91 precursor RNA is designated SEQ ID:77, and is provided hereinbelow with reference to the sequence listing part. Nucleotide sequence SEQ ID:77 is located at position 32319 relative to the genome of Vaccinia Virus.

VGAM91 precursor RNA folds onto itself, forming VGAM91 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM91 folded precursor RNA into VGAM91 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM91 RNA is designated SEQ ID:426, and is provided hereinbelow with reference to the sequence listing part.

VGAM91 host target gene, herein designated VGAM HOST TARGET GENE, encodes a corresponding messenger RNA, VGAM91 host target RNA, herein designated VGAM HOST TARGET RNA. VGAM91 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM91 RNA, herein designated VGAM RNA, binds complementarily to one or more host target binding sites located in untranslated regions of VGAM91 host target RNA, herein designated VGAM HOST TARGET RNA. This complementary binding is due to the fact that the nucleotide sequence of VGAM91 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM91 RNA, herein designated VGAM RNA, may have a different number of host target binding sites in untranslated regions of a VGAM91 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM91 RNA, herein designated VGAM RNA, to host target binding sites on VGAM91 host target RNA, herein designated VGAM HOST TARGET RNA, such as BINDING SITE I, BINDING SITE II and BINDING SITE III, inhibits translation of VGAM91 host target RNA into VGAM91 host target protein, herein designated VGAM HOST TARGET PROTEIN. VGAM host target protein is therefore outlined by a broken line.

It is appreciated that VGAM91 host target gene, herein designated VGAM HOST TARGET GENE, in fact represents a plurality of VGAM91 host target genes. The mRNA of each one of this plurality of VGAM91 host target genes comprises one or more host target binding sites, each having a nucleotide sequence which is at least partly complementary to VGAM91 RNA, herein designated VGAM RNA, and which when bound by VGAM91 RNA causes inhibition of translation of respective one or more VGAM91 host target proteins.

It is further appreciated by one skilled in the art that the mode of translational inhibition illustrated by FIG. 1 with specific reference to translational inhibition exerted by VGAM91 gene, herein designated VGAM GENE, on one or more VGAM91 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM91 is inhibition of expression of host target genes, as part of a novel viral mechanism of attacking a host. Accordingly, utilities of VGAM91 include diagnosis, prevention and treatment of viral infection by Vaccinia Virus. Specific functions, and accordingly utilities, of VGAM91 correlate with, and may be deduced from, the identity of the host target genes which VGAM91 binds and inhibits, and the function of these host target genes, as elaborated hereinbelow.

Nucleotide sequences of the VGAM91 precursor RNA, herein designated VGAM PRECURSOR RNA, and of the ‘diced’ VGAM91 RNA, herein designated VGAM RNA, and a schematic representation of the secondary folding of VGAM91 folded precursor RNA, herein designated VGAM FOLDED PRECURSOR RNA, of VGAM91 are further described hereinbelow with reference to Table 1.

Nucleotide sequences of host target binding sites, such as BINDING SITE-I, BINDING SITE-II and BINDING SITE-III of FIG. 1, found on VGAM91 host target RNA, and schematic representation of the complementarity of each of these host target binding sites to VGAM91 RNA, herein designated VGAM RNA, are described hereinbelow with reference to Table 2.

As mentioned hereinabove with reference to FIG. 1, a function of VGAM91 gene, herein designated VGAM is inhibition of expression of VGAM91 target genes. It is appreciated that specific functions, and accordingly utilities, of VGAM91 correlate with, and may be deduced from, the identity of the target genes which VGAM91 binds and inhibits, and the function of these target genes, as elaborated hereinbelow.

G Protein-coupled Receptor Kinase 6 (GPRK6, Accession NM_{—}002082) is a VGAM91 host target gene. GPRK6 BINDING SITE is HOST TARGET binding site found in the 3′ untranslated region of mRNA encoded by GPRK6, corresponding to a HOST TARGET binding site such as BINDING SITE I, BINDING SITE II or BINDING SITE III. Table 2 illustrates the complementarity of the nucleotide sequences of GPRK6 BINDING SITE, designated SEQ ID:898, to the nucleotide sequence of VGAM91 RNA, herein designated VGAM RNA, also designated SEQ ID:426.

A function of VGAM91 is therefore inhibition of G Protein-coupled Receptor Kinase 6 (GPRK6, Accession NM_{—}002082), a gene which regulates the G protein-coupled receptors. Accordingly, utilities of VGAM91 include diagnosis, prevention and treatment of diseases and clinical conditions associated with GPRK6. The function of GPRK6 has been established by previous studies. By PCR on neutrophil cDNA using primers based on sequences of known receptor kinases, Haribabu and Snyderman (1993) identified GPRK5 (OMIM Ref. No. 600870) and GPRK6 sequences. Using a fragment of the GPRK6 PCR clone to screen a cDNA library, they isolated a cDNA encoding GPRK6. Sequence analysis predicted that the 544-amino acid GPRK6 protein contains the conserved DLG (asp-leu-gly) and ENIL (glu-asn-ile-leu) motifs. Northern blot analysis detected 2.1- and 2.9-kb GPRK6 transcripts in all tissues tested, with strongest expression in placenta and skeletal muscle. By somatic cell hybrid analysis, Haribabu and Snyderman (1993) mapped the GPRK6 gene and a closely related gene to chromosomes 5 and 13, respectively. Bullrich et al. (1995) mapped GPRK6 to 5q35 by analysis of a rodent human hybrid panel. The GPRK6-related locus was found to map to 13pter-q21.

Full details of the abovementioned studies are described in the following publications, the disclosure of which are hereby incorporated by reference:
Bullrich, F.; Druck, T.; Kunapuli, P.; Gomez, J.; Gripp, K. W.; Schlegelberger, B.; Lasota, J.; Aronson, M.; Cannizzaro, L. A.; Huebner, K.; Benovic, J. L.: Chromosomal mapping of the genes GPRK5 and GPRK6 encoding G protein-coupled receptor kinases GRK5 and GRK6. Cytogenet. Cell Genet. 70: 250-254, 1995.; and
Haribabu, B.; Snyderman, R.: Identification of additional members of human G-protein-coupled receptor kinase multigene family. Proc. Nat. Acad. Sci. 90: 9398-9402, 1993.

Further studies establishing the function and utilities of GPRK6 are found in John Hopkins OMIM database record ID 600869, and in sited publications numbered 1613-1614 listed in the bibliography section hereinbelow, which are also hereby incorporated by reference. WWP1 (Accession XM_{—}087357) is another VGAM91 host target gene. WWP1 BINDING SITE is HOST TARGET binding site found in the 5′ untranslated region of mRNA encoded by WWP1, corresponding to a HOST TARGET binding site such as BINDING SITE I, BINDING SITE II or BINDING SITE III. Table 2 illustrates the complementarity of the nucleotide sequences of WWP1 BINDING SITE, designated SEQ ID:3164, to the nucleotide sequence of VGAM91 RNA, herein designated VGAM RNA, also designated SEQ ID:426.

Another function of VGAM91 is therefore inhibition of WWP1 (Accession XM_{—}087357). Accordingly, utilities of VGAM91 include diagnosis, prevention and treatment of diseases and clinical conditions associated with WWP1. LOC144559 (Accession XM_{—}084896) is another VGAM91 host target gene. LOC144559 BINDING SITE is HOST TARGET binding site found in the 5′ untranslated region of mRNA encoded by LOC144559, corresponding to a HOST TARGET binding site such as BINDING SITE I, BINDING SITE II or BINDING SITE III. Table 2 illustrates the complementarity of the nucleotide sequences of LOC144559 BINDING SITE, designated SEQ ID:3067, to the nucleotide sequence of VGAM91 RNA, herein designated VGAM RNA, also designated SEQ ID:426.

Another function of VGAM91 is therefore inhibition of LOC144559 (Accession XM_{—}084896). Accordingly, utilities of VGAM91 include diagnosis, prevention and treatment of diseases and clinical conditions associated with LOC144559. LOC200163 (Accession XM_{—}114136) is another VGAM91 host target gene. LOC200163 BINDING SITE is HOST TARGET binding site found in the 3′ untranslated region of mRNA encoded by LOC200163, corresponding to a HOST TARGET binding site such as BINDING SITE I, BINDING SITE II or BINDING SITE III. Table 2 illustrates the complementarity of the nucleotide sequences of LOC200163 BINDING SITE, designated SEQ ID:3440, to the nucleotide sequence of VGAM91 RNA, herein designated VGAM RNA, also designated SEQ ID:426.

Another function of VGAM91 is therefore inhibition of LOC200163 (Accession XM_{—}114136). Accordingly, utilities of VGAM91 include diagnosis, prevention and treatment of diseases and clinical conditions associated with LOC200163. LOC219918 (Accession XM_{—}166197) is another VGAM91 host target gene. LOC219918 BINDING SITE is HOST TARGET binding site found in the 5′ untranslated region of mRNA encoded by LOC219918, corresponding to a HOST TARGET binding site such as BINDING SITE I, BINDING SITE II or BINDING SITE III. Table 2 illustrates the complementarity of the nucleotide sequences of LOC219918 BINDING SITE, designated SEQ ID:3531, to the nucleotide sequence of VGAM91 RNA, herein designated VGAM RNA, also designated SEQ ID:426.

Another function of VGAM91 is therefore inhibition of LOC219918 (Accession XM_{—}166197). Accordingly, utilities of VGAM91 include diagnosis, prevention and treatment of diseases and clinical conditions associated with LOC219918. LOC220071 (Accession XM_{—}167848) is another VGAM91 host target gene. LOC220071 BINDING SITE is HOST TARGET binding site found in the 5′ untranslated region of mRNA encoded by LOC220071, corresponding to a HOST TARGET binding site such as BINDING SITE I, BINDING SITE II or BINDING SITE III. Table 2 illustrates the complementarity of the nucleotide sequences of LOC220071 BINDING SITE, designated SEQ ID:3603, to the nucleotide sequence of VGAM91 RNA, herein designated VGAM RNA, also designated SEQ ID:426.

Another function of VGAM91 is therefore inhibition of LOC220071 (Accession XM_{—}167848). Accordingly, utilities of VGAM91 include diagnosis, prevention and treatment of diseases and clinical conditions associated with LOC220071. LOC255515 (Accession XM_{—}171185) is another VGAM91 host target gene. LOC255515 BINDING SITE is HOST TARGET binding site found in the 3′ untranslated region of mRNA encoded by LOC255515, corresponding to a HOST TARGET binding site such as BINDING SITE I, BINDING SITE II or BINDING SITE III. Table 2 illustrates the complementarity of the nucleotide sequences of LOC255515 BINDING SITE, designated SEQ ID:3689, to the nucleotide sequence of VGAM91 RNA, herein designated VGAM RNA, also designated SEQ ID:426.

Another function of VGAM91 is therefore inhibition of LOC255515 (Accession XM_{—}171185). Accordingly, utilities of VGAM91 include diagnosis, prevention and treatment of diseases and clinical conditions associated with LOC255515. FIG. 1 further provides a conceptual description of a novel bioinformatically detected viral gene of the present invention, referred to here as Viral Genomic Address Messenger 92 (VGAM92) viral gene, which modulates expression of respective host target genes thereof, the function and utility of which host target genes is known in the art.

VGAM92 is a novel bioinformatically detected regulatory, non protein coding, viral micro RNA (miRNA) gene. The method by which VGAM92 was detected is described hereinabove with reference to FIGS. 1-8.

VGAM92 gene, herein designated VGAM GENE, is a viral gene contained in the genome of Vaccinia Virus. VGAM92 host target gene, herein designated VGAM HOST TARGET GENE, is a human gene contained in the human genome.

VGAM92 gene encodes a VGAM92 precursor RNA, herein designated VGAM PRECURSOR RNA. Similar to other miRNA genes, and unlike most ordinary genes, VGAM92 precursor RNA does not encode a protein. A nucleotide sequence identical or highly similar to the nucleotide sequence of VGAM92 precursor RNA is designated SEQ ID:78, and is provided hereinbelow with reference to the sequence listing part. Nucleotide sequence SEQ ID:78 is located at position 31579 relative to the genome of Vaccinia Virus.

VGAM92 precursor RNA folds onto itself, forming VGAM92 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM92 folded precursor RNA into VGAM92 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM92 RNA is designated SEQ ID:427, and is provided hereinbelow with reference to the sequence listing part.

VGAM92 host target gene, herein designated VGAM HOST TARGET GENE, encodes a corresponding messenger RNA, VGAM92 host target RNA, herein designated VGAM HOST TARGET RNA. VGAM92 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM92 RNA, herein designated VGAM RNA, binds complementarily to one or more host target binding sites located in untranslated regions of VGAM92 host target RNA, herein designated VGAM HOST TARGET RNA. This complementary binding is due to the fact that the nucleotide sequence of VGAM92 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM92 RNA, herein designated VGAM RNA, may have a different number of host target binding sites in untranslated regions of a VGAM92 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM92 RNA, herein designated VGAM RNA, to host target binding sites on VGAM92 host target RNA, herein designated VGAM HOST TARGET RNA, such as BINDING SITE I, BINDING SITE II and BINDING SITE III, inhibits translation of VGAM92 host target RNA into VGAM92 host target protein, herein designated VGAM HOST TARGET PROTEIN. VGAM host target protein is therefore outlined by a broken line.

It is appreciated that VGAM92 host target gene, herein designated VGAM HOST TARGET GENE, in fact represents a plurality of VGAM92 host target genes. The mRNA of each one of this plurality of VGAM92 host target genes comprises one or more host target binding sites, each having a nucleotide sequence which is at least partly complementary to VGAM92 RNA, herein designated VGAM RNA, and which when bound by VGAM92 RNA causes inhibition of translation of respective one or more VGAM92 host target proteins.

It is further appreciated by one skilled in the art that the mode of translational inhibition illustrated by FIG. 1 with specific reference to translational inhibition exerted by VGAM92 gene, herein designated VGAM GENE, on one or more VGAM92 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM92 is inhibition of expression of host target genes, as part of a novel viral mechanism of attacking a host. Accordingly, utilities of VGAM92 include diagnosis, prevention and treatment of viral infection by Vaccinia Virus. Specific functions, and accordingly utilities, of VGAM92 correlate with, and may be deduced from, the identity of the host target genes which VGAM92 binds and inhibits, and the function of these host target genes, as elaborated hereinbelow.

Nucleotide sequences of the VGAM92 precursor RNA, herein designated VGAM PRECURSOR RNA, and of the ‘diced’ VGAM92 RNA, herein designated VGAM RNA, and a schematic representation of the secondary folding of VGAM92 folded precursor RNA, herein designated VGAM FOLDED PRECURSOR RNA, of VGAM92 are further described hereinbelow with reference to Table 1.

Nucleotide sequences of host target binding sites, such as BINDING SITE-I, BINDING SITE-II and BINDING SITE-III of FIG. 1, found on VGAM92 host target RNA, and schematic representation of the complementarity of each of these host target binding sites to VGAM92 RNA, herein designated VGAM RNA, are described hereinbelow with reference to Table 2.

As mentioned hereinabove with reference to FIG. 1, a function of VGAM92 gene, herein designated VGAM is inhibition of expression of VGAM92 target genes. It is appreciated that specific functions, and accordingly utilities, of VGAM92 correlate with, and may be deduced from, the identity of the target genes which VGAM92 binds and inhibits, and the function of these target genes, as elaborated hereinbelow.

B-cell CLL/lymphoma 11B (zinc finger protein) (BCL11B, Accession NM_{—}022898) is a VGAM92 host target gene. BCL11B BINDING SITE is HOST TARGET binding site found in the 3′ untranslated region of mRNA encoded by BCL11B, corresponding to a HOST TARGET binding site such as BINDING SITE I, BINDING SITE II or BINDING SITE III. Table 2 illustrates the complementarity of the nucleotide sequences of BCL11B BINDING SITE, designated SEQ ID:2020, to the nucleotide sequence of VGAM92 RNA, herein designated VGAM RNA, also designated SEQ ID:427.

A function of VGAM92 is therefore inhibition of B-cell CLL/lymphoma 11B (zinc finger protein) (BCL11B, Accession NM_{—}022898). Accordingly, utilities of VGAM92 include diagnosis, prevention and treatment of diseases and clinical conditions associated with BCL11B. Membrane Component, Chromosome 17, Surface Marker 2 (ovarian carcinoma antigen CA125) (M17S2, Accession NM_{—}031858) is another VGAM92 host target gene. M17S2 BINDING SITE is HOST TARGET binding site found in the 5′ untranslated region of mRNA encoded by M17S2, corresponding to a HOST TARGET binding site such as BINDING SITE I, BINDING SITE II or BINDING SITE III. Table 2 illustrates the complementarity of the nucleotide sequences of M17S2 BINDING SITE, designated SEQ ID:2213, to the nucleotide sequence of VGAM92 RNA, herein designated VGAM RNA, also designated SEQ ID:427.

Another function of VGAM92 is therefore inhibition of Membrane Component, Chromosome 17, Surface Marker 2 (ovarian carcinoma antigen CA125) (M17S2, Accession NM_{—}031858), a gene which Contains a B-box/coiled coil motif. Accordingly, utilities of VGAM92 include diagnosis, prevention and treatment of diseases and clinical conditions associated with M17S2. The function of M17S2 has been established by previous studies. In an attempt to clone the CA125 gene (OMIM Ref. No. 606154), Campbell et al. (1994) isolated a cDNA from an expression library that mapped close to the BRCA1 locus (OMIM Ref. No. 113705) at 17q21.1. Closer investigation showed that it was within the smallest known region containing the BRCA1 gene. The predicted 966-amino acid polypeptide lacks the membrane protein characteristics expected for CA125 but does include a B-box/coiled coil motif present in many genes with transformation potential. Campbell et al. (1994) used fluorescence in situ hybridization to demonstrate mapping within the BRCA1 minimum region. YAC and cosmid clones were isolated and used to refine the location of this gene adjacent and proximal to the RNU2 locus (OMIM Ref. No. 180690). The exon structure of the gene was also determined. Extensive SSCP and sequence analysis of over 100 tumor and normal DNAs from familial and sporadic breast cancers and sporadic ovarian cancers failed to detect mutations in the coding region of this gene. Brown et al. (1994), who referred to the gene as 1A1-3B, showed that the transcription start site of M17S2 is 295 bp distal from the initiation site of BRCA1 and that the gene is transcribed divergently from BRCA1. The authors speculated that M17S2 may be involved in the regulation of transcription or translation of BRCA1. Brown et al. (1996) described the genomic region that encompasses both the BRCA1 and M17S2 genes. They found a tandem duplication of 30 kb that results in 2 copies of exons 1 and 2 of BRCA1 and exons 1 and 3 of M17S2.

Full details of the abovementioned studies are described in the following publications, the disclosure of which are hereby incorporated by reference:
Brown, M. A.; Xu, C.-F.; Nicolai, H.; Griffiths, B.; Chambers, J. A.; Black, D.; Solomon, E.: The 5-prime end of the BRCA1 gene lies within a duplicated region of human chromosome 17q21. Oncogene 12: 2507-2513, 1996.; and
Campbell, I. G.; Nicolai, H. M.; Foulkes, W. D.; Senger, G.; Stamp, G. W.; Allan, G.; Boyer, C.; Jones, K.; Bast, R. C., Jr.; Solomon, E.; Trowsdale, J.; Black, D. M.: A novel gene encodin.

Further studies establishing the function and utilities of M17S2 are found in John Hopkins OMIM database record ID 166945, and in sited publications numbered 389-391 listed in the bibliography section hereinbelow, which are also hereby incorporated by reference. CRFG (Accession XM_{—}040267) is another VGAM92 host target gene. CRFG BINDING SITE is HOST TARGET binding site found in the 3′ untranslated region of mRNA encoded by CRFG, corresponding to a HOST TARGET binding site such as BINDING SITE I, BINDING SITE II or BINDING SITE III. Table 2 illustrates the complementarity of the nucleotide sequences of CRFG BINDING SITE, designated SEQ ID:2775, to the nucleotide sequence of VGAM92 RNA, herein designated VGAM RNA, also designated SEQ ID:427.

Another function of VGAM92 is therefore inhibition of CRFG (Accession XM_{—}040267). Accordingly, utilities of VGAM92 include diagnosis, prevention and treatment of diseases and clinical conditions associated with CRFG. FLJ23360 (Accession NM_{—}023076) is another VGAM92 host target gene. FLJ23360 BINDING SITE is HOST TARGET binding site found in the 5′ untranslated region of mRNA encoded by FLJ23360, corresponding to a HOST TARGET binding site such as BINDING SITE I, BINDING SITE II or BINDING SITE III. Table 2 illustrates the complementarity of the nucleotide sequences of FLJ23360 BINDING SITE, designated SEQ ID:2030, to the nucleotide sequence of VGAM92 RNA, herein designated VGAM RNA, also designated SEQ ID:427.

Another function of VGAM92 is therefore inhibition of FLJ23360 (Accession NM_{—}023076). Accordingly, utilities of VGAM92 include diagnosis, prevention and treatment of diseases and clinical conditions associated with FLJ23360. KIAA0349 (Accession XM_{—}166449) is another VGAM92 host target gene. KIAA0349 BINDING SITE is HOST TARGET binding site found in the 3′ untranslated region of mRNA encoded by KIAA0349, corresponding to a HOST TARGET binding site such as BINDING SITE I, BINDING SITE II or BINDING SITE III. Table 2 illustrates the complementarity of the nucleotide sequences of KIAA0349 BINDING SITE, designated SEQ ID:3561, to the nucleotide sequence of VGAM92 RNA, herein designated VGAM RNA, also designated SEQ ID:427.

Another function of VGAM92 is therefore inhibition of KIAA0349 (Accession XM_{—}166449). Accordingly, utilities of VGAM92 include diagnosis, prevention and treatment of diseases and clinical conditions associated with KIAA0349. PRO2325 (Accession NM_{—}018523) is another VGAM92 host target gene. PRO2325 BINDING SITE is HOST TARGET binding site found in the 5′ untranslated region of mRNA encoded by PRO2325, corresponding to a HOST TARGET binding site such as BINDING SITE I, BINDING SITE II or BINDING SITE III. Table 2 illustrates the complementarity of the nucleotide sequences of PRO2325 BINDING SITE, designated SEQ ID:1836, to the nucleotide sequence of VGAM92 RNA, herein designated VGAM RNA, also designated SEQ ID:427.

Another function of VGAM92 is therefore inhibition of PRO2325 (Accession NM_{—}018523). Accordingly, utilities of VGAM92 include diagnosis, prevention and treatment of diseases and clinical conditions associated with PRO2325. SCYA16 (Accession NM_{—}004590) is another VGAM92 host target gene. SCYA16 BINDING SITE is HOST TARGET binding site found in the 3′ untranslated region of mRNA encoded by SCYA16, corresponding to a HOST TARGET binding site such as BINDING SITE I, BINDING SITE II or BINDING SITE III. Table 2 illustrates the complementarity of the nucleotide sequences of SCYA16 BINDING SITE, designated SEQ ID:1130, to the nucleotide sequence of VGAM92 RNA, herein designated VGAM RNA, also designated SEQ ID:427.

Another function of VGAM92 is therefore inhibition of SCYA16 (Accession NM_{—}004590). Accordingly, utilities of VGAM92 include diagnosis, prevention and treatment of diseases and clinical conditions associated with SCYA16. FIG. 1 further provides a conceptual description of a novel bioinformatically detected viral gene of the present invention, referred to here as Viral Genomic Address Messenger 93 (VGAM93) viral gene, which modulates expression of respective host target genes thereof, the function and utility of which host target genes is known in the art.

VGAM93 is a novel bioinformatically detected regulatory, non protein coding, viral micro RNA (miRNA) gene. The method by which VGAM93 was detected is described hereinabove with reference to FIGS. 1-8.

VGAM93 gene, herein designated VGAM GENE, is a viral gene contained in the genome of Vaccinia Virus. VGAM93 host target gene, herein designated VGAM HOST TARGET GENE, is a human gene contained in the human genome.

VGAM93 gene encodes a VGAM93 precursor RNA, herein designated VGAM PRECURSOR RNA. Similar to other miRNA genes, and unlike most ordinary genes, VGAM93 precursor RNA does not encode a protein. A nucleotide sequence identical or highly similar to the nucleotide sequence of VGAM93 precursor RNA is designated SEQ ID:79, and is provided hereinbelow with reference to the sequence listing part. Nucleotide sequence SEQ ID:79 is located at position 32553 relative to the genome of Vaccinia Virus.

VGAM93 precursor RNA folds onto itself, forming VGAM93 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM93 folded precursor RNA into VGAM93 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM93 RNA is designated SEQ ID:428, and is provided hereinbelow with reference to the sequence listing part.

VGAM93 host target gene, herein designated VGAM HOST TARGET GENE, encodes a corresponding messenger RNA, VGAM93 host target RNA, herein designated VGAM HOST TARGET RNA. VGAM93 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM93 RNA, herein designated VGAM RNA, binds complementarily to one or more host target binding sites located in untranslated regions of VGAM93 host target RNA, herein designated VGAM HOST TARGET RNA. This complementary binding is due to the fact that the nucleotide sequence of VGAM93 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM93 RNA, herein designated VGAM RNA, may have a different number of host target binding sites in untranslated regions of a VGAM93 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM93 RNA, herein designated VGAM RNA, to host target binding sites on VGAM93 host target RNA, herein designated VGAM HOST TARGET RNA, such as BINDING SITE I, BINDING SITE II and BINDING SITE III, inhibits translation of VGAM93 host target RNA into VGAM93 host target protein, herein designated VGAM HOST TARGET PROTEIN. VGAM host target protein is therefore outlined by a broken line.

It is appreciated that VGAM93 host target gene, herein designated VGAM HOST TARGET GENE, in fact represents a plurality of VGAM93 host target genes. The mRNA of each one of this plurality of VGAM93 host target genes comprises one or more host target binding sites, each having a nucleotide sequence which is at least partly complementary to VGAM93 RNA, herein designated VGAM RNA, and which when bound by VGAM93 RNA causes inhibition of translation of respective one or more VGAM93 host target proteins.

It is further appreciated by one skilled in the art that the mode of translational inhibition illustrated by FIG. 1 with specific reference to translational inhibition exerted by VGAM93 gene, herein designated VGAM GENE, on one or more VGAM93 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM93 is inhibition of expression of host target genes, as part of a novel viral mechanism of attacking a host. Accordingly, utilities of VGAM93 include diagnosis, prevention and treatment of viral infection by Vaccinia Virus. Specific functions, and accordingly utilities, of VGAM93 correlate with, and may be deduced from, the identity of the host target genes which VGAM93 binds and inhibits, and the function of these host target genes, as elaborated hereinbelow.

Nucleotide sequences of the VGAM93 precursor RNA, herein designated VGAM PRECURSOR RNA, and of the ‘diced’ VGAM93 RNA, herein designated VGAM RNA, and a schematic representation of the secondary folding of VGAM93 folded precursor RNA, herein designated VGAM FOLDED PRECURSOR RNA, of VGAM93 are further described hereinbelow with reference to Table 1.

Nucleotide sequences of host target binding sites, such as BINDING SITE-I, BINDING SITE-II and BINDING SITE-III of FIG. 1, found on VGAM93 host target RNA, and schematic representation of the complementarity of each of these host target binding sites to VGAM93 RNA, herein designated VGAM RNA, are described hereinbelow with reference to Table 2.

As mentioned hereinabove with reference to FIG. 1, a function of VGAM93 gene, herein designated VGAM is inhibition of expression of VGAM93 target genes. It is appreciated that specific functions, and accordingly utilities, of VGAM93 correlate with, and may be deduced from, the identity of the target genes which VGAM93 binds and inhibits, and the function of these target genes, as elaborated hereinbelow.

DJ-1 (Accession NM_{—}007262) is a VGAM93 host target gene. DJ-1 BINDING SITE is HOST TARGET binding site found in the 3′ untranslated region of mRNA encoded by DJ-1, corresponding to a HOST TARGET binding site such as BINDING SITE I, BINDING SITE II or BINDING SITE III. Table 2 illustrates the complementarity of the nucleotide sequences of DJ-1 BINDING SITE, designated SEQ ID:1375, to the nucleotide sequence of VGAM93 RNA, herein designated VGAM RNA, also designated SEQ ID:428.

A function of VGAM93 is therefore inhibition of DJ-1 (Accession NM_{—}007262). Accordingly, utilities of VGAM93 include diagnosis, prevention and treatment of diseases and clinical conditions associated with DJ-1. FLJ10535 (Accession NM_{—}018129) is another VGAM93 host target gene. FLJ10535 BINDING SITE is HOST TARGET binding site found in the 3′ untranslated region of mRNA encoded by FLJ10535, corresponding to a HOST TARGET binding site such as BINDING SITE I, BINDING SITE II or BINDING SITE III. Table 2 illustrates the complementarity of the nucleotide sequences of FLJ10535 BINDING SITE, designated SEQ ID: 1787, to the nucleotide sequence of VGAM93 RNA, herein designated VGAM RNA, also designated SEQ ID:428.

Another function of VGAM93 is therefore inhibition of FLJ10535 (Accession NM_{—}018129). Accordingly, utilities of VGAM93 include diagnosis, prevention and treatment of diseases and clinical conditions associated with FLJ10535. FLJ10539 (Accession NM_{—}018130) is another VGAM93 host target gene. FLJ10539 BINDING SITE is HOST TARGET binding site found in the 3′ untranslated region of mRNA encoded by FLJ10539, corresponding to a HOST TARGET binding site such as BINDING SITE I, BINDING SITE II or BINDING SITE III. Table 2 illustrates the complementarity of the nucleotide sequences of FLJ10539 BINDING SITE, designated SEQ ID:1788, to the nucleotide sequence of VGAM93 RNA, herein designated VGAM RNA, also designated SEQ ID:428.

Another function of VGAM93 is therefore inhibition of FLJ10539 (Accession NM_{—}018130). Accordingly, utilities of VGAM93 include diagnosis, prevention and treatment of diseases and clinical conditions associated with FLJ10539. Phosphatidylinositol (4,5) Bisphosphate 5-phosphatase, A (PIB5PA, Accession XM_{—}038489) is another VGAM93 host target gene. PIB5PA BINDING SITE is HOST TARGET binding site found in the 3′ untranslated region of mRNA encoded by PIB5PA, corresponding to a HOST TARGET binding site such as BINDING SITE I, BINDING SITE II or BINDING SITE III. Table 2 illustrates the complementarity of the nucleotide sequences of PIB5PA BINDING SITE, designated SEQ ID:2739, to the nucleotide sequence of VGAM93 RNA, herein designated VGAM RNA, also designated SEQ ID:428.

Another function of VGAM93 is therefore inhibition of Phosphatidylinositol (4,5) Bisphosphate 5-phosphatase, A (PIB5PA, Accession XM_{—}038489). Accordingly, utilities of VGAM93 include diagnosis, prevention and treatment of diseases and clinical conditions associated with PIB5PA. LOC149351 (Accession XM_{—}086503) is another VGAM93 host target gene. LOC149351 BINDING SITE is HOST TARGET binding site found in the 3′ untranslated region of mRNA encoded by LOC149351, corresponding to a HOST TARGET binding site such as BINDING SITE I, BINDING SITE II or BINDING SITE III. Table 2 illustrates the complementarity of the nucleotide sequences of LOC149351 BINDING SITE, designated SEQ ID:3130, to the nucleotide sequence of VGAM93 RNA, herein designated VGAM RNA, also designated SEQ ID:428.

Another function of VGAM93 is therefore inhibition of LOC149351 (Accession XM_{—}086503). Accordingly, utilities of VGAM93 include diagnosis, prevention and treatment of diseases and clinical conditions associated with LOC149351. LOC157858 (Accession XM_{—}098833) is another VGAM93 host target gene. LOC157858 BINDING SITE is HOST TARGET binding site found in the 5′ untranslated region of mRNA encoded by LOC157858, corresponding to a HOST TARGET binding site such as BINDING SITE I, BINDING SITE II or BINDING SITE III. Table 2 illustrates the complementarity of the nucleotide sequences of LOC157858 BINDING SITE, designated SEQ ID:3384, to the nucleotide sequence of VGAM93 RNA, herein designated VGAM RNA, also designated SEQ ID:428.

Another function of VGAM93 is therefore inhibition of LOC157858 (Accession XM_{—}098833). Accordingly, utilities of VGAM93 include diagnosis, prevention and treatment of diseases and clinical conditions associated with LOC157858. FIG. 1 further provides a conceptual description of a novel bioinformatically detected viral gene of the present invention, referred to here as Viral Genomic Address Messenger 94 (VGAM94) viral gene, which modulates expression of respective host target genes thereof, the function and utility of which host target genes is known in the art.

VGAM94 is a novel bioinformatically detected regulatory, non protein coding, viral micro RNA (miRNA) gene. The method by which VGAM94 was detected is described hereinabove with reference to FIGS. 1-8.

VGAM94 gene, herein designated VGAM GENE, is a viral gene contained in the genome of Vaccinia Virus. VGAM94 host target gene, herein designated VGAM HOST TARGET GENE, is a human gene contained in the human genome.

VGAM94 gene encodes a VGAM94 precursor RNA, herein designated VGAM PRECURSOR RNA. Similar to other miRNA genes, and unlike most ordinary genes, VGAM94 precursor RNA does not encode a protein. A nucleotide sequence identical or highly similar to the nucleotide sequence of VGAM94 precursor RNA is designated SEQ ID:80, and is provided hereinbelow with reference to the sequence listing part. Nucleotide sequence SEQ ID:80 is located at position 32481 relative to the genome of Vaccinia Virus.

VGAM94 precursor RNA folds onto itself, forming VGAM94 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM94 folded precursor RNA into VGAM94 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM94 RNA is designated SEQ ID:429, and is provided hereinbelow with reference to the sequence listing part.

VGAM94 host target gene, herein designated VGAM HOST TARGET GENE, encodes a corresponding messenger RNA, VGAM94 host target RNA, herein designated VGAM HOST TARGET RNA. VGAM94 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM94 RNA, herein designated VGAM RNA, binds complementarily to one or more host target binding sites located in untranslated regions of VGAM94 host target RNA, herein designated VGAM HOST TARGET RNA. This complementary binding is due to the fact that the nucleotide sequence of VGAM94 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM94 RNA, herein designated VGAM RNA, may have a different number of host target binding sites in untranslated regions of a VGAM94 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM94 RNA, herein designated VGAM RNA, to host target binding sites on VGAM94 host target RNA, herein designated VGAM HOST TARGET RNA, such as BINDING SITE I, BINDING SITE II and BINDING SITE III, inhibits translation of VGAM94 host target RNA into VGAM94 host target protein, herein designated VGAM HOST TARGET PROTEIN. VGAM host target protein is therefore outlined by a broken line.

It is appreciated that VGAM94 host target gene, herein designated VGAM HOST TARGET GENE, in fact represents a plurality of VGAM94 host target genes. The mRNA of each one of this plurality of VGAM94 host target genes comprises one or more host target binding sites, each having a nucleotide sequence which is at least partly complementary to VGAM94 RNA, herein designated VGAM RNA, and which when bound by VGAM94 RNA causes inhibition of translation of respective one or more VGAM94 host target proteins.

It is further appreciated by one skilled in the art that the mode of translational inhibition illustrated by FIG. 1 with specific reference to translational inhibition exerted by VGAM94 gene, herein designated VGAM GENE, on one or more VGAM94 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM94 is inhibition of expression of host target genes, as part of a novel viral mechanism of attacking a host. Accordingly, utilities of VGAM94 include diagnosis, prevention and treatment of viral infection by Vaccinia Virus. Specific functions, and accordingly utilities, of VGAM94 correlate with, and may be deduced from, the identity of the host target genes which VGAM94 binds and inhibits, and the function of these host target genes, as elaborated hereinbelow.

Nucleotide sequences of the VGAM94 precursor RNA, herein designated VGAM PRECURSOR RNA, and of the ‘diced’ VGAM94 RNA, herein designated VGAM RNA, and a schematic representation of the secondary folding of VGAM94 folded precursor RNA, herein designated VGAM FOLDED PRECURSOR RNA, of VGAM94 are further described hereinbelow with reference to Table 1.

Nucleotide sequences of host target binding sites, such as BINDING SITE-I, BINDING SITE-II and BINDING SITE-III of FIG. 1, found on VGAM94 host target RNA, and schematic representation of the complementarity of each of these host target binding sites to VGAM94 RNA, herein designated VGAM RNA, are described hereinbelow with reference to Table 2.

As mentioned hereinabove with reference to FIG. 1, a function of VGAM94 gene, herein designated VGAM is inhibition of expression of VGAM94 target genes. It is appreciated that specific functions, and accordingly utilities, of VGAM94 correlate with, and may be deduced from, the identity of the target genes which VGAM94 binds and inhibits, and the function of these target genes, as elaborated hereinbelow.

Discs, Large (Drosophila) Homolog 5 (DLG5, Accession XM_{—}096398) is a VGAM94 host target gene. DLG5 BINDING SITE is HOST TARGET binding site found in the 5′ untranslated region of mRNA encoded by DLG5, corresponding to a HOST TARGET binding site such as BINDING SITE I, BINDING SITE II or BINDING SITE III. Table 2 illustrates the complementarity of the nucleotide sequences of DLG5 BINDING SITE, designated SEQ ID:3265, to the nucleotide sequence of VGAM94 RNA, herein designated VGAM RNA, also designated SEQ ID:429.

A function of VGAM94 is therefore inhibition of Discs, Large (Drosophila) Homolog 5 (DLG5, Accession XM_{—}096398), a gene which may transmit extracellular signals to inhibit cell proliferation. Accordingly, utilities of VGAM94 include diagnosis, prevention and treatment of diseases and clinical conditions associated with DLG5. The function of DLG5 has been established by previous studies. Vertebrate homologs of the Drosophila discs large (dlg) gene are members of the MAGUK (membrane-associated guanylate kinase) family. See 602887. MAGUK proteins contain PDZ motifs, an SH3 domain, and a guanylate kinase (GUK)-homologous region. Both the PDZ and GUK domains are thought to contribute to protein-protein interactions. By searching an EST database for sequences related to Drosophila dlg, Nakamura et al. (1998) identified cDNAs encoding a novel human homolog. Northern blot analysis revealed that the 9.4-kb transcript was highly expressed in placenta and prostate, as well as in several other tissues, leading the authors to designate the gene PDLG (placenta and prostate DLG). An additional 8.8-kb PDLG mRNA was detected in thyroid. The predicted 859-amino acid PDLG protein contains 3 PDZ domains, an SH3 domain, and a GUK region. PDLG is 45% and 40% identical to DLG1 (OMIM Ref. No. 601014) and Drosophila dlg, respectively. Western blot analysis of extracts of human prostate tissue and various cell lines showed that PDLG has an apparent molecular mass of 105 kD. Immunofluorescence experiments indicated that PDLG is localized at the plasma membrane and cytoplasm, and is expressed in the gland epithelial cells of normal prostate tissue but not in prostate cell lines. Using a yeast 2-hybrid screen, Nakamura et al. (1998) determined that PDLG interacts with the GUK domain of p55 (MPP1; 305360), a palmitoylated erythrocyte membrane MAGUK protein. The authors suggested that PDLG and p55 form a heteromeric MAGUK complex at the plasma membrane and cluster various intracellular molecules to play roles in maintaining the structure of epithelial cells and transmitting extracellular signals to the membrane and cytoskeleton. Independently, Nagase et al. (1998) identified KIAA0583, a DLG5 cDNA. By radiation hybrid analysis, they mapped the DLG5 gene to chromosome 10. Using the same technique, Nakamura et al. (1998) refined the localization of the DLG5 gene to 10q23.

Full details of the abovementioned studies are described in the following publications, the disclosure of which are hereby incorporated by reference:
Nagase, T.; Ishikawa, K.; Miyajima, N.; Tanaka, A.; Kotani, H.; Nomura, N.; Ohara, O.: Prediction of the coding sequences of unidentified human genes. IX. The complete sequences of 100 new cDNA clones from brain which can code for large proteins in vitro. DNA Res. 5: 31-39, 1998.; and
Nakamura, H.; Sudo, T.; Tsuiki, H.; Miyake, H.; Morisaki, T.; Sasaki, J.; Masuko, N.; Kochi, M.; Ushio, Y.; Saya, H.: Identification of a novel human homolog of the Drosophila dlg, P-d.

Further studies establishing the function and utilities of DLG5 are found in John Hopkins OMIM database record ID 604090, and in sited publications numbered 1563 listed in the bibliography section hereinbelow, which are also hereby incorporated by reference. Dishevelled, Dsh Homolog 3 (Drosophila) (DVL3, Accession NM_{—}004423) is another VGAM94 host target gene. DVL3 BINDING SITE is HOST TARGET binding site found in the 3′ untranslated region of mRNA encoded by DVL3, corresponding to a HOST TARGET binding site such as BINDING SITE I, BINDING SITE II or BINDING SITE III. Table 2 illustrates the complementarity of the nucleotide sequences of DVL3 BINDING SITE, designated SEQ ID:1112, to the nucleotide sequence of VGAM94 RNA, herein designated VGAM RNA, also designated SEQ ID:429.

Another function of VGAM94 is therefore inhibition of Dishevelled, Dsh Homolog 3 (Drosophila) (DVL3, Accession NM_{—}004423), a gene which regulates cell proliferation. Accordingly, utilities of VGAM94 include diagnosis, prevention and treatment of diseases and clinical conditions associated with DVL3. The function of DVL3 has been established by previous studies. The Drosophila dishevelled gene (dsh) encodes a cytoplasmic phosphoprotein (Klingensmith et al., 1994) that regulates cell proliferation, acting as a transducer molecule for developmental processes, including segmentation and neuroblast specification. Pizzuti et al. (1996) noted that dsh is required for the function of the wingless gene product wg, a segment polarity gene homologous to the mammalian protooncogene WNT1 (OMIM Ref. No. 164820). Pizzuti et al. (1996) reported the isolation and chromosomal mapping of 2 human dsh homologs, designated DVL1 (OMIM Ref. No. 601365) and DVL3 by them. The human dsh homologs were isolated from a fetal brain cDNA library. DVL3 encodes a predicted 716-amino acid polypeptide that shows 74% nucleotide homology with human DVL1 and 71% homology with the mouse Dvl1 gene. DVL1 and DVL3 share 64% amino acid identity. Pizzuti et al. (1996) reported that homology is particularly high in the N-terminal region and that there is more divergence in the C-terminal regions. PCR carried out using DNA from rodent human somatic cell hybrids and DVL3 specific primers led to the assignment of DVL3 to human chromosome 3. Pizzuti et al. (1996) regionally assigned DVL3 to band 3q27 using fluorescence in situ hybridization. Hybridization of poly(A) mRNA with the DVL3 cDNA revealed a 2.9-kb transcript with abundant expression in skeletal muscle, pancreas and heart. They also detected 5.9-kb and 5.0-kb transcripts in skeletal muscle, adult liver, adult heart, pancreas, and placenta. The 5.9-kb form was abundant in fetal tissues but the 5.0-kb form was absent from these tissues. Pizzuti et al. (1996) noted that Charcot-Marie-Tooth type 2B (OMIM Ref. No. 600882) maps to chromosome 3q. Bui et al. (1997) also isolated human DVL3, which shares 98% amino acid identity with mouse Dvl3 and 49% with Drosophila dsh. The authors confirmed the chromosomal localization at 3p27. Semenov and Snyder (1997) isolated 3 human genes encoding proteins homologous to Drosophila dsh. The cDNA sequence of DVL3 reported by Semenov and Snyder (1997) differs from the previously reported sequences deposited in GenBank. Bui et al. (1997) detected expression of DVL3 mRNA in B cells, breast, kidney, bladder, endometrium, and 2 primary endometrial cultures. It was detected equally in normal human breast tissues and tumors and in colorectal samples of normal tissues, polyps, and tumors.

Full details of the abovementioned studies are described in the following publications, the disclosure of which are hereby incorporated by reference:
Pizzuti, A.; Amati, F.; Calabrese, G.; Mari, A.; Colosimo, A; Silani, V.; Giardino, L.; Ratti, A.; Penso, D.; Calza, L.; Palka, G.; Scarlato, G.; Novelli, G.; Dallapicolla, B.: cDNA characterization and chromosomal mapping of two human homologs of the Drosophila dishevelled polarity gene. Hum. Molec. Genet. 5: 953-958, 1996.; and
Semenov, M. V.; Snyder, M.: Human dishevelled genes constitute a DHR-containing multigene family. Genomics 42: 302-310, 1997.

Further studies establishing the function and utilities of DVL3 are found in John Hopkins OMIM database record ID 601368, and in sited publications numbered 2213-1597 listed in the bibliography section hereinbelow, which are also hereby incorporated by reference. Ectonucleotide Pyrophosphatase/phosphodiesterase 3 (ENPP3, Accession NM_{—}005021) is another VGAM94 host target gene. ENPP3 BINDING SITE is HOST TARGET binding site found in the 3′ untranslated region of mRNA encoded by ENPP3, corresponding to a HOST TARGET binding site such as BINDING SITE I, BINDING SITE II or BINDING SITE III. Table 2 illustrates the complementarity of the nucleotide sequences of ENPP3 BINDING SITE, designated SEQ ID:1173, to the nucleotide sequence of VGAM94 RNA, herein designated VGAM RNA, also designated SEQ ID:429.

Another function of VGAM94 is therefore inhibition of Ectonucleotide Pyrophosphatase/phosphodiesterase 3 (ENPP3, Accession NM_{—}005021). Accordingly, utilities of VGAM94 include diagnosis, prevention and treatment of diseases and clinical conditions associated with ENPP3. Fanconi Anemia, Complementation Group F (FANCF, Accession NM_{—}022725) is another VGAM94 host target gene. FANCF BINDING SITE is HOST TARGET binding site found in the 3′ untranslated region of mRNA encoded by FANCF, corresponding to a HOST TARGET binding site such as BINDING SITE I, BINDING SITE II or BINDING SITE III. Table 2 illustrates the complementarity of the nucleotide sequences of FANCF BINDING SITE, designated SEQ ID:1998, to the nucleotide sequence of VGAM94 RNA, herein designated VGAM RNA, also designated SEQ ID:429.

Another function of VGAM94 is therefore inhibition of Fanconi Anemia, Complementation Group F (FANCF, Accession NM_{—}022725). Accordingly, utilities of VGAM94 include diagnosis, prevention and treatment of diseases and clinical conditions associated with FANCF. Proto-cadherin 11 X-linked (PCDH11X, Accession NM_{—}032968) is another VGAM94 host target gene. PCDH11X BINDING SITE1 and PCDH11X BINDING SITE2 are HOST TARGET binding sites found in untranslated regions of mRNA encoded by PCDH11X, corresponding to HOST TARGET binding sites such as BINDING SITE I, BINDING SITE II or BINDING SITE III. Table 2 illustrates the complementarity of the nucleotide sequences of PCDH11X BINDING SITE1 and PCDH11X BINDING SITE2, designated SEQ ID:2298 and SEQ ID:2300 respectively, to the nucleotide sequence of VGAM94 RNA, herein designated VGAM RNA, also designated SEQ ID:429.

Another function of VGAM94 is therefore inhibition of Proto-cadherin 11 X-linked (PCDH11X, Accession NM_{—}032968), a gene which is thought to play a fundamental role in cell-cell recognition essential for the segmental development and function of the central nervous system. Accordingly, utilities of VGAM94 include diagnosis, prevention and treatment of diseases and clinical conditions associated with PCDH11X. The function of PCDH11X and its association with various diseases and clinical conditions, has been established by previous studies, as described hereinabove with reference to VGAM49. FLJ10539 (Accession NM_{—}018130) is another VGAM94 host target gene. FLJ10539 BINDING SITE is HOST TARGET binding site found in the 3′ untranslated region of mRNA encoded by FLJ10539, corresponding to a HOST TARGET binding site such as BINDING SITE I, BINDING SITE II or BINDING SITE III. Table 2 illustrates the complementarity of the nucleotide sequences of FLJ10539 BINDING SITE, designated SEQ ID:1789, to the nucleotide sequence of VGAM94 RNA, herein designated VGAM RNA, also designated SEQ ID:429.

Another function of VGAM94 is therefore inhibition of FLJ10539 (Accession NM_{—}018130). Accordingly, utilities of VGAM94 include diagnosis, prevention and treatment of diseases and clinical conditions associated with FLJ10539. FLJ10898 (Accession XM_{—}002486) is another VGAM94 host target gene. FLJ10898 BINDING SITE is HOST TARGET binding site found in the 3′ untranslated region of mRNA encoded by FLJ10898, corresponding to a HOST TARGET binding site such as BINDING SITE I, BINDING SITE II or BINDING SITE III. Table 2 illustrates the complementarity of the nucleotide sequences of FLJ10898 BINDING SITE, designated SEQ ID:2528, to the nucleotide sequence of VGAM94 RNA, herein designated VGAM RNA, also designated SEQ ID:429.

Another function of VGAM94 is therefore inhibition of FLJ10898 (Accession XM_{—}002486). Accordingly, utilities of VGAM94 include diagnosis, prevention and treatment of diseases and clinical conditions associated with FLJ10898. KIAA0680 (Accession NM_{—}014721) is another VGAM94 host target gene. KIAA0680 BINDING SITE is HOST TARGET binding site found in the 3′ untranslated region of mRNA encoded by KIAA0680, corresponding to a HOST TARGET binding site such as BINDING SITE I, BINDING SITE II or BINDING SITE III. Table 2 illustrates the complementarity of the nucleotide sequences of KIAA0680 BINDING SITE, designated SEQ ID:1530, to the nucleotide sequence of VGAM94 RNA, herein designated VGAM RNA, also designated SEQ ID:429.

Another function of VGAM94 is therefore inhibition of KIAA0680 (Accession NM_{—}014721). Accordingly, utilities of VGAM94 include diagnosis, prevention and treatment of diseases and clinical conditions associated with KIAA0680. KIAA1804 (Accession XM_{—}045864) is another VGAM94 host target gene. KIAA1804 BINDING SITE is HOST TARGET binding site found in the 3′ untranslated region of mRNA encoded by KIAA1804, corresponding to a HOST TARGET binding site such as BINDING SITE I, BINDING SITE II or BINDING SITE III. Table 2 illustrates the complementarity of the nucleotide sequences of KIAA1804 BINDING SITE, designated SEQ ID:2868, to the nucleotide sequence of VGAM94 RNA, herein designated VGAM RNA, also designated SEQ ID:429.

Another function of VGAM94 is therefore inhibition of KIAA1804 (Accession XM_{—}045864). Accordingly, utilities of VGAM94 include diagnosis, prevention and treatment of diseases and clinical conditions associated with KIAA1804. MO25 (Accession NM_{—}016289) is another VGAM94 host target gene. MO25 BINDING SITE is HOST TARGET binding site found in the 3′ untranslated region of mRNA encoded by MO25, corresponding to a HOST TARGET binding site such as BINDING SITE I, BINDING SITE II or BINDING SITE III. Table 2 illustrates the complementarity of the nucleotide sequences of MO25 BINDING SITE, designated SEQ ID: 1677, to the nucleotide sequence of VGAM94 RNA, herein designated VGAM RNA, also designated SEQ ID:429.

Another function of VGAM94 is therefore inhibition of MO25 (Accession NM_{—}016289). Accordingly, utilities of VGAM94 include diagnosis, prevention and treatment of diseases and clinical conditions associated with MO25. RAB33B, Member RAS Oncogene Family (RAB33B, Accession NM_{—}031296) is another VGAM94 host target gene. RAB33B BINDING SITE is HOST TARGET binding site found in the 3′ untranslated region of mRNA encoded by RAB33B, corresponding to a HOST TARGET binding site such as BINDING SITE I, BINDING SITE II or BINDING SITE III. Table 2 illustrates the complementarity of the nucleotide sequences of RAB33B BINDING SITE, designated SEQ ID:2190, to the nucleotide sequence of VGAM94 RNA, herein designated VGAM RNA, also designated SEQ ID:429.

Another function of VGAM94 is therefore inhibition of RAB33B, Member RAS Oncogene Family (RAB33B, Accession NM_{—}031296). Accordingly, utilities of VGAM94 include diagnosis, prevention and treatment of diseases and clinical conditions associated with RAB33B. RoXaN (Accession NM_{—}025013) is another VGAM94 host target gene. RoXaN BINDING SITE is HOST TARGET binding site found in the 3′ untranslated region of mRNA encoded by RoXaN, corresponding to a HOST TARGET binding site such as BINDING SITE I, BINDING SITE II or BINDING SITE III. Table 2 illustrates the complementarity of the nucleotide sequences of RoXaN BINDING SITE, designated SEQ ID:2128, to the nucleotide sequence of VGAM94 RNA, herein designated VGAM RNA, also designated SEQ ID:429.

Another function of VGAM94 is therefore inhibition of RoXaN (Accession NM_{—}025013). Accordingly, utilities of VGAM94 include diagnosis, prevention and treatment of diseases and clinical conditions associated with RoXaN. FIG. 1 further provides a conceptual description of a novel bioinformatically detected viral gene of the present invention, referred to here as Viral Genomic Address Messenger 95 (VGAM95) viral gene, which modulates expression of respective host target genes thereof, the function and utility of which host target genes is known in the art.

VGAM95 is a novel bioinformatically detected regulatory, non protein coding, viral micro RNA (miRNA) gene. The method by which VGAM95 was detected is described hereinabove with reference to FIGS. 1-8.

VGAM95 gene, herein designated VGAM GENE, is a viral gene contained in the genome of Vaccinia Virus. VGAM95 host target gene, herein designated VGAM HOST TARGET GENE, is a human gene contained in the human genome.

VGAM95 gene encodes a VGAM95 precursor RNA, herein designated VGAM PRECURSOR RNA. Similar to other miRNA genes, and unlike most ordinary genes, VGAM95 precursor RNA does not encode a protein. A nucleotide sequence identical or highly similar to the nucleotide sequence of VGAM95 precursor RNA is designated SEQ ID:81, and is provided hereinbelow with reference to the sequence listing part. Nucleotide sequence SEQ ID:81 is located at position 31304 relative to the genome of Vaccinia Virus.

VGAM95 precursor RNA folds onto itself, forming VGAM95 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM95 folded precursor RNA into VGAM95 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM95 RNA is designated SEQ ID:430, and is provided hereinbelow with reference to the sequence listing part.

VGAM95 host target gene, herein designated VGAM HOST TARGET GENE, encodes a corresponding messenger RNA, VGAM95 host target RNA, herein designated VGAM HOST TARGET RNA. VGAM95 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM95 RNA, herein designated VGAM RNA, binds complementarily to one or more host target binding sites located in untranslated regions of VGAM95 host target RNA, herein designated VGAM HOST TARGET RNA. This complementary binding is due to the fact that the nucleotide sequence of VGAM95 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM95 RNA, herein designated VGAM RNA, may have a different number of host target binding sites in untranslated regions of a VGAM95 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM95 RNA, herein designated VGAM RNA, to host target binding sites on VGAM95 host target RNA, herein designated VGAM HOST TARGET RNA, such as BINDING SITE I, BINDING SITE II and BINDING SITE III, inhibits translation of VGAM95 host target RNA into VGAM95 host target protein, herein designated VGAM HOST TARGET PROTEIN. VGAM host target protein is therefore outlined by a broken line.

It is appreciated that VGAM95 host target gene, herein designated VGAM HOST TARGET GENE, in fact represents a plurality of VGAM95 host target genes. The mRNA of each one of this plurality of VGAM95 host target genes comprises one or more host target binding sites, each having a nucleotide sequence which is at least partly complementary to VGAM95 RNA, herein designated VGAM RNA, and which when bound by VGAM95 RNA causes inhibition of translation of respective one or more VGAM95 host target proteins.

It is further appreciated by one skilled in the art that the mode of translational inhibition illustrated by FIG. 1 with specific reference to translational inhibition exerted by VGAM95 gene, herein designated VGAM GENE, on one or more VGAM95 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM95 is inhibition of expression of host target genes, as part of a novel viral mechanism of attacking a host. Accordingly, utilities of VGAM95 include diagnosis, prevention and treatment of viral infection by Vaccinia Virus. Specific functions, and accordingly utilities, of VGAM95 correlate with, and may be deduced from, the identity of the host target genes which VGAM95 binds and inhibits, and the function of these host target genes, as elaborated hereinbelow.

Nucleotide sequences of the VGAM95 precursor RNA, herein designated VGAM PRECURSOR RNA, and of the ‘diced’ VGAM95 RNA, herein designated VGAM RNA, and a schematic representation of the secondary folding of VGAM95 folded precursor RNA, herein designated VGAM FOLDED PRECURSOR RNA, of VGAM95 are further described hereinbelow with reference to Table 1.

Nucleotide sequences of host target binding sites, such as BINDING SITE-I, BINDING SITE-II and BINDING SITE-III of FIG. 1, found on VGAM95 host target RNA, and schematic representation of the complementarity of each of these host target binding sites to VGAM95 RNA, herein designated VGAM RNA, are described hereinbelow with reference to Table 2.

As mentioned hereinabove with reference to FIG. 1, a function of VGAM95 gene, herein designated VGAM is inhibition of expression of VGAM95 target genes. It is appreciated that specific functions, and accordingly utilities, of VGAM95 correlate with, and may be deduced from, the identity of the target genes which VGAM95 binds and inhibits, and the function of these target genes, as elaborated hereinbelow.

Kinesin Family Member 13A (KIF13A, Accession NM_{—}022113) is a VGAM95 host target gene. KIF13A BINDING SITE is HOST TARGET binding site found in the 3′ untranslated region of mRNA encoded by KIF13A, corresponding to a HOST TARGET binding site such as BINDING SITE I, BINDING SITE II or BINDING SITE III. Table 2 illustrates the complementarity of the nucleotide sequences of KIF13A BINDING SITE, designated SEQ ID: 1979, to the nucleotide sequence of VGAM95 RNA, herein designated VGAM RNA, also designated SEQ ID:430.

A function of VGAM95 is therefore inhibition of Kinesin Family Member 13A (KIF13A, Accession NM_{—}022113), a gene which plays a role of motor for anterograde axonal transport of synaptic vesicle precursors (by similarity). Accordingly, utilities of VGAM95 include diagnosis, prevention and treatment of diseases and clinical conditions associated with KIF13A. The function of KIF13A has been established by previous studies. Intracellular transport mediated by kinesin super family proteins (KIFs; OMIM Ref. No. 604535) is a highly regulated process. Nakagawa et al. (2000) identified mouse Kif13a, a plus end-directed microtubule-dependent motor protein that associates with beta-1 adaptin (AP1B1; 600157), a subunit of the AP1 adaptor complex. The 1,749-amino acid mouse Kif13a protein contains an N-terminal motor domain and an AF6/cno domain. The authors found that the cargo vesicles of Kif13a contain AP1 and mannose 6-phosphate receptor (M6PR; 154540). Overexpression of Kif13a resulted in mislocalization of AP1 and M6PR, and functional blockade of Kif13a reduced cell-surface expression of M6PR. Thus, the authors concluded that KIF13A transports M6PR-containing vesicles and targets M6PR from the trans-Golgi network to the plasma membrane via direct interaction with the AP1 adaptor complex. Scott (2000) mapped the KIF13A gene to chromosome 6 based on sequence similarity between a KIF13A sequence (GenBank AK025303) and the chromosome 6 clone RP11-500C11 (GenBank AL138724).

Full details of the abovementioned studies are described in the following publications, the disclosure of which are hereby incorporated by reference:
Nakagawa, T.; Setou, M.; Seog, D.-H.; Ogasawara, K.; Dohmae, N.; Takio, K.; Hirokawa, N.: A novel motor, KIF13A, transports mannose-6-phosphate receptor to plasma membrane through direct interaction with AP-1 complex. Cell 103: 569-581, 2000.; and
Scott, A. F.: Personal Communication. Baltimore, Md., Nov. 29, 2000.

Further studies establishing the function and utilities of KIF13A are found in John Hopkins OMIM database record ID 605433, and in sited publications numbered 990-991 listed in the bibliography section hereinbelow, which are also hereby incorporated by reference. Lysyl Oxidase-like 3 (LOXL3, Accession NM_{—}032603) is another VGAM95 host target gene. LOXL3 BINDING SITE is HOST TARGET binding site found in the 3′ untranslated region of mRNA encoded by LOXL3, corresponding to a HOST TARGET binding site such as BINDING SITE I, BINDING SITE II or BINDING SITE III. Table 2 illustrates the complementarity of the nucleotide sequences of LOXL3 BINDING SITE, designated SEQ ID:2264, to the nucleotide sequence of VGAM95 RNA, herein designated VGAM RNA, also designated SEQ ID:430.

Another function of VGAM95 is therefore inhibition of Lysyl Oxidase-like 3 (LOXL3, Accession NM_{—}032603), a gene which is expressed in many tissues, the highest levels seen in placenta, heart, ovary, testis, small intestine and spleen. Accordingly, utilities of VGAM95 include diagnosis, prevention and treatment of diseases and clinical conditions associated with LOXL3. The function of LOXL3 has been established by previous studies. By database searching for sequences homologous to mouse Lor2, Jourdan-Le Saux et al. (2001) isolated a LOXL3 cDNA encoding a deduced 754-amino acid protein with a molecular mass of 83 kD. The protein contains all structural characteristics of the LOX enzyme family, including a copper-binding site with 4 histidyl residues; lysyl and tyrosyl residues and conserved motifs surrounding these sites; and the cytokine receptor-like domain. Like LOXL2 (OMIM Ref. No. 606663), it has 4 scavenger receptor cysteine-rich (SRCR) domains in its N terminus. LOXL3 has a signal peptide sequence, indicating a possible extracellular location, 5 potential N-glycosylation sites, and a predicted procollagen C-proteinase (BMP1; 112264) cleavage site (gly-asp-asp) that is also present in LOX. By Northern blot analysis, Jourdan-Le Saux et al. (2001) detected expression of an approximately 3.3-kb LOXL3 transcript in all tissues tested, with high expression in heart and uterus and low expression in kidney, skeletal muscle, and placenta. Unlike other members of the LOX gene family, LOXL3 was found to be expressed at low levels in all sections of the central nervous system, with highest levels in spinal cord and medulla. By Northern blot analysis, Maki and Kivirikko (2001) detected highest expression of a 3.1-kb LOXL3 transcript in placenta, heart, ovary, testis, small intestine, and spleen Jourdan-Le Saux et al. (2001) determined that the LOXL3 gene contains 14 exons and spans approximately 21 kb. By EST database searching, they identified splice variants lacking exons 5 and 8

Full details of the abovementioned studies are described in the following publications, the disclosure of which are hereby incorporated by reference:
Jourdan-Le Saux, C.; Tomsche, A.; Ujfalusi, A.; Jia, L.; Csiszar, K.: Central nervous system, uterus, heart, and leukocyte expression of the LOXL3 gene, encoding a novel lysyl oxidase-like protein. Genomics 74: 211-218, 2001.; and
Jourdan-Le Saux, C.; Tomsche, A.; Ujfalusi, A.; Jia, L.; Csiszar, K.: Central nervous system, uterus, heart, and leukocyte expression of the LOXL3 gene, encoding a novel lysyl oxidase-like.

Further studies establishing the function and utilities of LOXL3 are found in John Hopkins OMIM database record ID 607163, and in sited publications numbered 1328-1329 listed in the bibliography section hereinbelow, which are also hereby incorporated by reference. DKFZp5471014 (Accession NM_{—}020217) is another VGAM95 host target gene. DKFZp5471014 BINDING SITE is HOST TARGET binding site found in the 5′ untranslated region of mRNA encoded by DKFZp5471014, corresponding to a HOST TARGET binding site such as BINDING SITE I, BINDING SITE II or BINDING SITE III. Table 2 illustrates the complementarity of the nucleotide sequences of DKFZp5471014 BINDING SITE, designated SEQ ID:1901, to the nucleotide sequence of VGAM95 RNA, herein designated VGAM RNA, also designated SEQ ID:430.

Another function of VGAM95 is therefore inhibition of DKFZp5471014 (Accession NM_{—}020217). Accordingly, utilities of VGAM95 include diagnosis, prevention and treatment of diseases and clinical conditions associated with DKFZp5471014. Spir-1 (Accession XM_{—}035640) is another VGAM95 host target gene. Spir-1 BINDING SITE is HOST TARGET binding site found in the 3′ untranslated region of mRNA encoded by Spir-1, corresponding to a HOST TARGET binding site such as BINDING SITE I, BINDING SITE II or BINDING SITE III. Table 2 illustrates the complementarity of the nucleotide sequences of Spir-1 BINDING SITE, designated SEQ ID:2699, to the nucleotide sequence of VGAM95 RNA, herein designated VGAM RNA, also designated SEQ ID:430.

Another function of VGAM95 is therefore inhibition of Spir-1 (Accession XM_{—}035640). Accordingly, utilities of VGAM95 include diagnosis, prevention and treatment of diseases and clinical conditions associated with Spir-1. FIG. 1 further provides a conceptual description of a novel bioinformatically detected viral gene of the present invention, referred to here as Viral Genomic Address Messenger 96 (VGAM96) viral gene, which modulates expression of respective host target genes thereof, the function and utility of which host target genes is known in the art.

VGAM96 is a novel bioinformatically detected regulatory, non protein coding, viral micro RNA (miRNA) gene. The method by which VGAM96 was detected is described hereinabove with reference to FIGS. 1-8.

VGAM96 gene, herein designated VGAM GENE, is a viral gene contained in the genome of Vaccinia Virus. VGAM96 host target gene, herein designated VGAM HOST TARGET GENE, is a human gene contained in the human genome.

VGAM96 gene encodes a VGAM96 precursor RNA, herein designated VGAM PRECURSOR RNA. Similar to other miRNA genes, and unlike most ordinary genes, VGAM96 precursor RNA does not encode a protein. A nucleotide sequence identical or highly similar to the nucleotide sequence of VGAM96 precursor RNA is designated SEQ ID:82, and is provided hereinbelow with reference to the sequence listing part. Nucleotide sequence SEQ ID:82 is located at position 30764 relative to the genome of Vaccinia Virus.

VGAM96 precursor RNA folds onto itself, forming VGAM96 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM96 folded precursor RNA into VGAM96 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM96 RNA is designated SEQ ID:431, and is provided hereinbelow with reference to the sequence listing part.

VGAM96 host target gene, herein designated VGAM HOST TARGET GENE, encodes a corresponding messenger RNA, VGAM96 host target RNA, herein designated VGAM HOST TARGET RNA. VGAM96 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM96 RNA, herein designated VGAM RNA, binds complementarily to one or more host target binding sites located in untranslated regions of VGAM96 host target RNA, herein designated VGAM HOST TARGET RNA. This complementary binding is due to the fact that the nucleotide sequence of VGAM96 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM96 RNA, herein designated VGAM RNA, may have a different number of host target binding sites in untranslated regions of a VGAM96 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM96 RNA, herein designated VGAM RNA, to host target binding sites on VGAM96 host target RNA, herein designated VGAM HOST TARGET RNA, such as BINDING SITE I, BINDING SITE II and BINDING SITE III, inhibits translation of VGAM96 host target RNA into VGAM96 host target protein, herein designated VGAM HOST TARGET PROTEIN. VGAM host target protein is therefore outlined by a broken line.

It is appreciated that VGAM96 host target gene, herein designated VGAM HOST TARGET GENE, in fact represents a plurality of VGAM96 host target genes. The mRNA of each one of this plurality of VGAM96 host target genes comprises one or more host target binding sites, each having a nucleotide sequence which is at least partly complementary to VGAM96 RNA, herein designated VGAM RNA, and which when bound by VGAM96 RNA causes inhibition of translation of respective one or more VGAM96 host target proteins.

It is further appreciated by one skilled in the art that the mode of translational inhibition illustrated by FIG. 1 with specific reference to translational inhibition exerted by VGAM96 gene, herein designated VGAM GENE, on one or more VGAM96 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM96 is inhibition of expression of host target genes, as part of a novel viral mechanism of attacking a host. Accordingly, utilities of VGAM96 include diagnosis, prevention and treatment of viral infection by Vaccinia Virus. Specific functions, and accordingly utilities, of VGAM96 correlate with, and may be deduced from, the identity of the host target genes which VGAM96 binds and inhibits, and the function of these host target genes, as elaborated hereinbelow.

Nucleotide sequences of the VGAM96 precursor RNA, herein designated VGAM PRECURSOR RNA, and of the ‘diced’ VGAM96 RNA, herein designated VGAM RNA, and a schematic representation of the secondary folding of VGAM96 folded precursor RNA, herein designated VGAM FOLDED PRECURSOR RNA, of VGAM96 are further described hereinbelow with reference to Table 1.

Nucleotide sequences of host target binding sites, such as BINDING SITE-I, BINDING SITE-II and BINDING SITE-III of FIG. 1, found on VGAM96 host target RNA, and schematic representation of the complementarity of each of these host target binding sites to VGAM96 RNA, herein designated VGAM RNA, are described hereinbelow with reference to Table 2.

As mentioned hereinabove with reference to FIG. 1, a function of VGAM96 gene, herein designated VGAM is inhibition of expression of VGAM96 target genes. It is appreciated that specific functions, and accordingly utilities, of VGAM96 correlate with, and may be deduced from, the identity of the target genes which VGAM96 binds and inhibits, and the function of these target genes, as elaborated hereinbelow.

Glutamate-cysteine Ligase, Catalytic Subunit (GCLC, Accession NM_{—}001498) is a VGAM96 host target gene. GCLC BINDING SITE is HOST TARGET binding site found in the 3′ untranslated region of mRNA encoded by GCLC, corresponding to a HOST TARGET binding site such as BINDING SITE I, BINDING SITE II or BINDING SITE III. Table 2 illustrates the complementarity of the nucleotide sequences of GCLC BINDING SITE, designated SEQ ID:833, to the nucleotide sequence of VGAM96 RNA, herein designated VGAM RNA, also designated SEQ ID:431.

A function of VGAM96 is therefore inhibition of Glutamate-cysteine Ligase, Catalytic Subunit (GCLC, Accession NM_{—}001498). Accordingly, utilities of VGAM96 include diagnosis, prevention and treatment of diseases and clinical conditions associated with GCLC. FLJ21324 (Accession XM_{—}165988) is another VGAM96 host target gene. FLJ21324 BINDING SITE is HOST TARGET binding site found in the 5′ untranslated region of mRNA encoded by FLJ21324, corresponding to a HOST TARGET binding site such as BINDING SITE I, BINDING SITE II or BINDING SITE III. Table 2 illustrates the complementarity of the nucleotide sequences of FLJ21324 BINDING SITE, designated SEQ ID:3513, to the nucleotide sequence of VGAM96 RNA, herein designated VGAM RNA, also designated SEQ ID:431.

Another function of VGAM96 is therefore inhibition of FLJ21324 (Accession XM_{—}165988). Accordingly, utilities of VGAM96 include diagnosis, prevention and treatment of diseases and clinical conditions associated with FLJ21324. KIAA0193 (Accession NM_{—}014766) is another VGAM96 host target gene. KIAA0193 BINDING SITE is HOST TARGET binding site found in the 3′ untranslated region of mRNA encoded by KIAA0193, corresponding to a HOST TARGET binding site such as BINDING SITE I, BINDING SITE II or BINDING SITE III. Table 2 illustrates the complementarity of the nucleotide sequences of KIAA0193 BINDING SITE, designated SEQ ID:1543, to the nucleotide sequence of VGAM96 RNA, herein designated VGAM RNA, also designated SEQ ID:431.

Another function of VGAM96 is therefore inhibition of KIAA0193 (Accession NM_{—}014766). Accordingly, utilities of VGAM96 include diagnosis, prevention and treatment of diseases and clinical conditions associated with KIAA0193. LOC91097 (Accession XM_{—}035977) is another VGAM96 host target gene. LOC91097 BINDING SITE is HOST TARGET binding site found in the 3′ untranslated region of mRNA encoded by LOC91097, corresponding to a HOST TARGET binding site such as BINDING SITE I, BINDING SITE II or BINDING SITE III. Table 2 illustrates the complementarity of the nucleotide sequences of LOC91097 BINDING SITE, designated SEQ ID:2705, to the nucleotide sequence of VGAM96 RNA, herein designated VGAM RNA, also designated SEQ ID:431.

Another function of VGAM96 is therefore inhibition of LOC91097 (Accession XM_{—}035977). Accordingly, utilities of VGAM96 include diagnosis, prevention and treatment of diseases and clinical conditions associated with LOC91097. FIG. 1 further provides a conceptual description of a novel bioinformatically detected viral gene of the present invention, referred to here as Viral Genomic Address Messenger 97 (VGAM97) viral gene, which modulates expression of respective host target genes thereof, the function and utility of which host target genes is known in the art.

VGAM97 is a novel bioinformatically detected regulatory, non protein coding, viral micro RNA (miRNA) gene. The method by which VGAM97 was detected is described hereinabove with reference to FIGS. 1-8.

VGAM97 gene, herein designated VGAM GENE, is a viral gene contained in the genome of Vaccinia Virus. VGAM97 host target gene, herein designated VGAM HOST TARGET GENE, is a human gene contained in the human genome.

VGAM97 gene encodes a VGAM97 precursor RNA, herein designated VGAM PRECURSOR RNA. Similar to other miRNA genes, and unlike most ordinary genes, VGAM97 precursor RNA does not encode a protein. A nucleotide sequence identical or highly similar to the nucleotide sequence of VGAM97 precursor RNA is designated SEQ ID:83, and is provided hereinbelow with reference to the sequence listing part. Nucleotide sequence SEQ ID:83 is located at position 31794 relative to the genome of Vaccinia Virus.

VGAM97 precursor RNA folds onto itself, forming VGAM97 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM97 folded precursor RNA into VGAM97 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM97 RNA is designated SEQ ID:432, and is provided hereinbelow with reference to the sequence listing part.

VGAM97 host target gene, herein designated VGAM HOST TARGET GENE, encodes a corresponding messenger RNA, VGAM97 host target RNA, herein designated VGAM HOST TARGET RNA. VGAM97 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM97 RNA, herein designated VGAM RNA, binds complementarily to one or more host target binding sites located in untranslated regions of VGAM97 host target RNA, herein designated VGAM HOST TARGET RNA. This complementary binding is due to the fact that the nucleotide sequence of VGAM97 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM97 RNA, herein designated VGAM RNA, may have a different number of host target binding sites in untranslated regions of a VGAM97 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM97 RNA, herein designated VGAM RNA, to host target binding sites on VGAM97 host target RNA, herein designated VGAM HOST TARGET RNA, such as BINDING SITE I, BINDING SITE II and BINDING SITE III, inhibits translation of VGAM97 host target RNA into VGAM97 host target protein, herein designated VGAM HOST TARGET PROTEIN. VGAM host target protein is therefore outlined by a broken line.

It is appreciated that VGAM97 host target gene, herein designated VGAM HOST TARGET GENE, in fact represents a plurality of VGAM97 host target genes. The mRNA of each one of this plurality of VGAM97 host target genes comprises one or more host target binding sites, each having a nucleotide sequence which is at least partly complementary to VGAM97 RNA, herein designated VGAM RNA, and which when bound by VGAM97 RNA causes inhibition of translation of respective one or more VGAM97 host target proteins.

It is further appreciated by one skilled in the art that the mode of translational inhibition illustrated by FIG. 1 with specific reference to translational inhibition exerted by VGAM97 gene, herein designated VGAM GENE, on one or more VGAM97 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM97 is inhibition of expression of host target genes, as part of a novel viral mechanism of attacking a host. Accordingly, utilities of VGAM97 include diagnosis, prevention and treatment of viral infection by Vaccinia Virus. Specific functions, and accordingly utilities, of VGAM97 correlate with, and may be deduced from, the identity of the host target genes which VGAM97 binds and inhibits, and the function of these host target genes, as elaborated hereinbelow.

Nucleotide sequences of the VGAM97 precursor RNA, herein designated VGAM PRECURSOR RNA, and of the ‘diced’ VGAM97 RNA, herein designated VGAM RNA, and a schematic representation of the secondary folding of VGAM97 folded precursor RNA, herein designated VGAM FOLDED PRECURSOR RNA, of VGAM97 are further described hereinbelow with reference to Table 1.

Nucleotide sequences of host target binding sites, such as BINDING SITE-I, BINDING SITE-II and BINDING SITE-III of FIG. 1, found on VGAM97 host target RNA, and schematic representation of the complementarity of each of these host target binding sites to VGAM97 RNA, herein designated VGAM RNA, are described hereinbelow with reference to Table 2.

As mentioned hereinabove with reference to FIG. 1, a function of VGAM97 gene, herein designated VGAM is inhibition of expression of VGAM97 target genes. It is appreciated that specific functions, and accordingly utilities, of VGAM97 correlate with, and may be deduced from, the identity of the target genes which VGAM97 binds and inhibits, and the function of these target genes, as elaborated hereinbelow.

A Disintegrin-like and Metalloprotease (reprolysin type) with Thrombospondin Type 1 Motif, 5 (aggrecanase-2) (ADAMTS5, Accession NM_{—}007038) is a VGAM97 host target gene. ADAMTS5 BINDING SITE is HOST TARGET binding site found in the 3′ untranslated region of mRNA encoded by ADAMTS5, corresponding to a HOST TARGET binding site such as BINDING SITE I, BINDING SITE II or BINDING SITE III. Table 2 illustrates the complementarity of the nucleotide sequences of ADAMTS5 BINDING SITE, designated SEQ ID:1353, to the nucleotide sequence of VGAM97 RNA, herein designated VGAM RNA, also designated SEQ ID:432.

A function of VGAM97 is therefore inhibition of A Disintegrin-like and Metalloprotease (reprolysin type) with Thrombospondin Type 1 Motif, 5 (aggrecanase-2) (ADAMTS5, Accession NM_{—}007038), a gene which cleaves aggrecan, a cartilage proteoglycan, and may be involved in its turnover. Accordingly, utilities of VGAM97 include diagnosis, prevention and treatment of diseases and clinical conditions associated with ADAMTS5. The function of ADAMTS5 and its association with various diseases and clinical conditions, has been established by previous studies, as described hereinabove with reference to VGAM49. BCRP2 (Accession XM_{—}031102) is another VGAM97 host target gene. BCRP2 BINDING SITE is HOST TARGET binding site found in the 3′ untranslated region of mRNA encoded by BCRP2, corresponding to a HOST TARGET binding site such as BINDING SITE I, BINDING SITE II or BINDING SITE III. Table 2 illustrates the complementarity of the nucleotide sequences of BCRP2 BINDING SITE, designated SEQ ID:2633, to the nucleotide sequence of VGAM97 RNA, herein designated VGAM RNA, also designated SEQ ID:432.

Another function of VGAM97 is therefore inhibition of BCRP2 (Accession XM_{—}031102). Accordingly, utilities of VGAM97 include diagnosis, prevention and treatment of diseases and clinical conditions associated with BCRP2. Interleukin 13 Receptor, Alpha 1 (IL13RA1, Accession NM_{—}001560) is another VGAM97 host target gene. IL13RA1 BINDING SITE is HOST TARGET binding site found in the 3′ untranslated region of mRNA encoded by IL13RA1, corresponding to a HOST TARGET binding site such as BINDING SITE I, BINDING SITE II or BINDING SITE III. Table 2 illustrates the complementarity of the nucleotide sequences of IL13RA1 BINDING SITE, designated SEQ ID:835, to the nucleotide sequence of VGAM97 RNA, herein designated VGAM RNA, also designated SEQ ID:432.

Another function of VGAM97 is therefore inhibition of Interleukin 13 Receptor, Alpha 1 (IL13RA1, Accession NM_{—}001560), a gene which binds il-13 with a low affinity. together with il-4r-alpha can form a functional receptor for il-13. Accordingly, utilities of VGAM97 include diagnosis, prevention and treatment of diseases and clinical conditions associated with IL13RA1. The function of IL13RA1 has been established by previous studies. Interleukin-4 (IL4; 147780) and interleukin-13 (IL13; 147683) are 2 cytokines that are secreted by activated T cells and have similar effects on monocytes and B cells. Zurawski et al. (1993) demonstrated that the IL4 receptor (OMIM Ref. No. 147781) is a complex of at least 2 components. They described a mutant form of human IL4 that competitively antagonizes both human IL4 and human IL13. The amino acid sequences of IL4 and IL13 are approximately 30% homologous, and circular dichroism spectroscopy demonstrates that both proteins have a highly alpha-helical structure. IL13 competitively inhibited binding of IL4 to functional human IL4 receptors expressed on a cell line that responds to both IL4 and IL13. The binding of IL4 to an IL4-responsive cell line that does not respond to IL13, and the binding of IL4 to cloned IL4R ligand binding protein expressed on heterologous cells, were not inhibited by IL13. The results demonstrated that IL4 and IL13 share a receptor component that is important for signal transduction. Hilton et al. (1996) reviewed these and other data suggesting a model of IL4 and IL13 receptor composition and function Heinzmann et al. (2000) determined that a variant of human IL13 (OMIM Ref. No. 147683), arg110 to gin (OMIM Ref. No. A4464G), associated with asthma in case-control populations from Britain and Japan (peak odds ratio (OR)=2.31, 95% confidence interval, 1.33-4.00); the variant also predicted asthma and higher serum IL13 levels in a general, Japanese pediatric population. The authors referred to this variant as gln110 to arg. Immunohistochemistry demonstrated that both subunits of IL13R are prominently expressed in bronchial epithelium and smooth muscle from asthmatic subjects. Detailed molecular modeling analyses indicated that residue 110 of IL13 is important in the internal constitution of the ligand and crucial in ligand-receptor interaction. A noncoding variant of IL13R-alpha 1, 1398A-G, associated primarily with high IgE levels (OR=3.38 in males, 1.10 in females) rather than asthma

Full details of the abovementioned studies are described in the following publications, the disclosure of which are hereby incorporated by reference:
Hilton, D. J.; Zhang, J.-G.; Metcalf, D.; Alexander, W. S.; Nicola, N. A.; Willson, T. A.: Cloning and characterization of a binding subunit of the interleukin 13 receptor that is also a component of the interleukin 4 receptor. Proc. Nat. Acad. Sci. 93: 497-501, 1996.; and
Heinzmann, H.; Mao, X.-Q.; Akaiwa, M.; Kreomer, R. T.; Gao, P.-S.; Ohshima, K.; Umeshita, R.; Abe, Y.; Braun, S.; Yamashita, T.; Roberts, M. H.; Sugimoto, R.; and 20 others: Genetic var.

Further studies establishing the function and utilities of IL13RA1 are found in John Hopkins OMIM database record ID 300119, and in sited publications numbered 2434-2435, 144, 243 and 2669 listed in the bibliography section hereinbelow, which are also hereby incorporated by reference. Interleukin 1, Alpha (IL1A, Accession XM_{—}031221) is another VGAM97 host target gene. IL1A BINDING SITE is HOST TARGET binding site found in the 3′ untranslated region of mRNA encoded by IL1A, corresponding to a HOST TARGET binding site such as BINDING SITE I, BINDING SITE II or BINDING SITE III. Table 2 illustrates the complementarity of the nucleotide sequences of IL1A BINDING SITE, designated SEQ ID:2634, to the nucleotide sequence of VGAM97 RNA, herein designated VGAM RNA, also designated SEQ ID:432.

Another function of VGAM97 is therefore inhibition of Interleukin 1, Alpha (IL1A, Accession XM_{—}031221), a gene which stimulates thymocyte proliferation by inducing il-2 release, b-cell maturation and proliferation, and fibroblast growth factor activity. Accordingly, utilities of VGAM97 include diagnosis, prevention and treatment of diseases and clinical conditions associated with IL1A. The function of IL1A has been established by previous studies. There are 2 structurally distinct forms of IL1: IL1(alpha), which is the acidic form with p15, and IL1(beta) (IL1B; 147720), the neutral form with p17. Both are 17-kD proteins coded by separate genes. The IL1A gene has 10,206 bp with 7 exons and 6 introns (Furutani et al., 1986). By Southern transfer analysis of DNAs from human-rodent somatic cell hybrids, Modi et al. (1988) assigned the IL1A gene to chromosome 2. Regional localization to 2q13-q21 was achieved by in situ hybridization. Lafage et al. (1989) confirmed assignment to 2q13 by in situ hybridization. The IL1A and IL1B proteins, which are synthesized by a variety of cell types including activated macrophages, keratinocytes, stimulated B lymphocytes, and fibroblasts, are potent mediators of inflammation and immunity. Lord et al. (1991) demonstrated that both the alpha and beta forms, but particularly the beta form, are transcribed in polymorphonuclear leukocytes stimulated with LPS. Both IL1A and IL1B stimulate osteoclast activity in vitro and are potent bone resorbing factors. Sabatino et al. (1988) studied the effects of 72-hour subcutaneous infusions of interleukins 1-alpha and -beta on plasma, calcium, and bone morphology. Both interleukins 1 caused a marked, dose-dependent increase in plasma calcium. Increased numbers of osteoclasts and bone resorption surfaces were observed on quantitative histomorphometry of bone. The results suggest a role for IL1 in the modulation of extracellular fluid calcium homeostasis. Hogquist et al. (1991) demonstrated that interleukin-1 is involved in apoptosis (cell death). Both the alpha and the beta forms are released as a consequence of cell injury regardless of the insult Ki et al. (2001) analyzed the IL1A-889 C/T genotype of 126 Korean patients with AD and found no significant difference in allele frequencies between patients and controls. Interestingly, there were no T/T homozygotes in the entire study population

Full details of the abovementioned studies are described in the following publications, the disclosure of which are hereby incorporated by reference:
Hogquist, K. A.; Nett, M. A.; Unanue, E. R.; Chaplin, D. D.: Interleukin 1 is processed and released during apoptosis. Proc. Nat. Acad. Sci. 88: 8485-8489, 1991.; and
Ki, C.-S.; Na, D. L.; Kim, D. K.; Kim, H. J.; Kim, J.-W.: Lack of association of the interleukin-1-alpha gene polymorphism with Alzheimer's disease in a Korean population. (Letter) Ann.

Further studies establishing the function and utilities of IL1A are found in John Hopkins OMIM database record ID 147760, and in sited publications numbered 923-938, 920-922, 939, 256 and 2627-2630 listed in the bibliography section hereinbelow, which are also hereby incorporated by reference. ISL1 Transcription Factor, LIM/homeodomain, (islet-1) (ISL1, Accession NM_{—}002202) is another VGAM97 host target gene. ISL1 BINDING SITE is HOST TARGET binding site found in the 3′ untranslated region of mRNA encoded by ISL1, corresponding to a HOST TARGET binding site such as BINDING SITE I, BINDING SITE II or BINDING SITE III. Table 2 illustrates the complementarity of the nucleotide sequences of ISL1 BINDING SITE, designated SEQ ID:906, to the nucleotide sequence of VGAM97 RNA, herein designated VGAM RNA, also designated SEQ ID:432.

Another function of VGAM97 is therefore inhibition of ISL1 Transcription Factor, LIM/homeodomain, (islet-1) (ISL1, Accession NM_{—}002202), a gene which binds to one of the cis-acting domain of the insulin gene enhancer. Accordingly, utilities of VGAM97 include diagnosis, prevention and treatment of diseases and clinical conditions associated with ISL1. The function of ISL1 has been established by previous studies. Because insulin deficiency, either relative or absolute, is a cardinal feature of noninsulin-dependent diabetes mellitus (NIDDM; 125853), Tanizawa et al. (1994) investigated the possible involvement of mutations in genes that regulate insulin production. Rat Isl1 was the first insulin enhancer-binding protein to be isolated; Tanizawa et al. (1994) used the rat gene to isolate a partial human ISL1 cDNA and subsequently to isolate genomic clones. A simple sequence repeat was found in the ISL1 gene. PCR amplification of this region of genomic DNA revealed 12 alleles in St. Louis African-Americans (heterozygosity=0.87), 14 alleles in black Nigerians (heterozygosity=0.89), 8 alleles in Japanese (heterozygosity=0.69), and 8 alleles in Caucasians (heterozygosity=0.81). Allelic frequencies in the gene did not differ between patients with NIDDM and nondiabetic control subjects in 2 black populations Shimomura et al. (2000) found a nonsense mutation (Q310X) in the ISL1 gene in a Japanese patient with type II diabetes and a strong family history. The mutation led to decreased activity of the islet-1 transcription factor and thus may have been pathogenic. However, as indicated by Fajans et al. (2001), additional genetic and clinical studies were required to determine whether mutations in ISL1 are the cause of another subtype of MODY

Full details of the abovementioned studies are described in the following publications, the disclosure of which are hereby incorporated by reference:
Shimomura, H.; Sanke, T.; Hanabusa, T.; Tsunoda, K.; Furuta, H.; Nanjo, K.: Nonsense mutation of islet-1 gene (Q310X) found in a type 2 diabetic patient with a strong family history. Diabetes 49: 1597-1600, 2000.; and
Tanizawa, Y.; Riggs, A. C.; Dagogo-Jack, S.; Vaxillaire, M.; Froguel, P.; Liu, L.; Donis-Keller, H.; Permutt, M. A.: Isolation of the human LIM/homeodomain gene islet-1 and identification.

Further studies establishing the function and utilities of ISL1 are found in John Hopkins OMIM database record ID 600366, and in sited publications numbered 2810 listed in the bibliography section hereinbelow, which are also hereby incorporated by reference. Microphthalmia-associated Transcription Factor (MITF, Accession NM_{—}000248) is another VGAM97 host target gene. MITF BINDING SITE is HOST TARGET binding site found in the 3′ untranslated region of mRNA encoded by MITF, corresponding to a HOST TARGET binding site such as BINDING SITE I, BINDING SITE II or BINDING SITE III. Table 2 illustrates the complementarity of the nucleotide sequences of MITF BINDING SITE, designated SEQ ID:721, to the nucleotide sequence of VGAM97 RNA, herein designated VGAM RNA, also designated SEQ ID:432.

Another function of VGAM97 is therefore inhibition of Microphthalmia-associated Transcription Factor (MITF, Accession NM_{—}000248), a gene which waardenburg syndrome type 2a, tietz syndrome. Accordingly, utilities of VGAM97 include diagnosis, prevention and treatment of diseases and clinical conditions associated with MITF. The function of MITF has been established by previous studies. Tassabehji et al. (1995) concluded that Waardenburg syndrome type II is heterogeneous, with about 20% of cases caused by mutations in MITF. In the mouse, mi mutations can be dominant or recessive. Dominant alleles are believed to work by a dominant-negative effect. A protein with intact helix-loop-helix and zipper sequences but defective DNA binding or transactivation domains sequesters the normal gene products in inactive dimers. Mutations that prevent dimerization are recessive. Tassabehji et al. (1995) noted that most of the mouse mutations are recessive and most of the human mutations in MITF appear to be dominant. They concluded that MITF is another example of a gene like RET (OMIM Ref. No. 164761) or PAX3 in which humans are more sensitive than mice to gene dosage effects in heterozygotes. Mutations in MITF and PAX3, encoding transcription factors, are responsible for Waardenburg syndrome type II and WS1/WS3, respectively. Tachibana et al. (1996) showed that MITF transactivates the gene for tyrosinase (see OMIM Ref. No. 606933), a key enzyme for melanogenesis, and is critically involved in melanocyte differentiation. Absence of melanocytes affects pigmentation in the skin, hair, and eyes, and hearing function in the cochlea. Therefore, hypopigmentation and hearing loss in WS2 are likely to be the results of an anomaly of melanocyte differentiation caused by MITF mutations. However, the molecular mechanism by which PAX3 mutations cause the auditory-pigmentary symptoms in WS1/WS3 had not been explained. Watanabe et al. (1998) showed that PAX3, a transcription factor with a paired domain and a homeodomain, transactivates the MITF promoter. They further showed that PAX3 proteins associated with WS1 in either the paired domain or the homeodomain failed to recognize and transactivate the MITF promoter. These results provided evidence that PAX3 directly regulates MITF, and suggested that the failure of this regulation due to PAX3 mutations causes the auditory-pigmentary symptoms in at least some individuals with WS1.

Full details of the abovementioned studies are described in the following publications, the disclosure of which are hereby incorporated by reference:
Tachibana, M.; Takeda, K.; Nobukuni, Y.; Urabe, K.; Long, J. E.; Meyers, K. A.; Aaronson, S. A.; Miki, T.: Ectopic expression of MITF, a gene for Waardenburg syndrome type 2, converts fibroblasts to cells with melanocytes characteristics. Nature Genet. 14: 50-54, 1996.; and
Tassabehji, M.; Newton, V. E.; Liu, X.-Z.; Brady, A.; Donnai, D.; Krajewska-Walasek, M.; Murday, V.; Norman, A.; Obersztyn, E.; Reardon, W.; Rice, J. C.; Trembath, R.; Wieacker, P.; Wh.

Further studies establishing the function and utilities of MITF are found in John Hopkins OMIM database record ID 156845, and in sited publications numbered 786, 2814-789, 525, 790, 2815, 2470, 2816-2475, 725, 247 and 2817-2478 listed in the bibliography section hereinbelow, which are also hereby incorporated by reference. Neutrophil Cytosolic Factor 2 (65 kDa, chronic granulomatous disease, autosomal 2) (NCF2, Accession NM_{—}000433) is another VGAM97 host target gene. NCF2 BINDING SITE is HOST TARGET binding site found in the 3′ untranslated region of mRNA encoded by NCF2, corresponding to a HOST TARGET binding site such as BINDING SITE I, BINDING SITE II or BINDING SITE III. Table 2 illustrates the complementarity of the nucleotide sequences of NCF2 BINDING SITE, designated SEQ ID:742, to the nucleotide sequence of VGAM97 RNA, herein designated VGAM RNA, also designated SEQ ID:432.

Another function of VGAM97 is therefore inhibition of Neutrophil Cytosolic Factor 2 (65 kDa, chronic granulomatous disease, autosomal 2) (NCF2, Accession NM_{—}000433). Accordingly, utilities of VGAM97 include diagnosis, prevention and treatment of diseases and clinical conditions associated with NCF2. Neural Precursor Cell Expressed, Developmentally Down-regulated 4-like (NEDD4L, Accession NM_{—}015277) is another VGAM97 host target gene. NEDD4L BINDING SITE is HOST TARGET binding site found in the 3′ untranslated region of mRNA encoded by NEDD4L, corresponding to a HOST TARGET binding site such as BINDING SITE I, BINDING SITE II or BINDING SITE III. Table 2 illustrates the complementarity of the nucleotide sequences of NEDD4L BINDING SITE, designated SEQ ID: 1618, to the nucleotide sequence of VGAM97 RNA, herein designated VGAM RNA, also designated SEQ ID:432.

Another function of VGAM97 is therefore inhibition of Neural Precursor Cell Expressed, Developmentally Downregulated 4-like (NEDD4L, Accession NM_{—}015277), a gene which may play a role in the regulation of epithelial sodium channel function. Accordingly, utilities of VGAM97 include diagnosis, prevention and treatment of diseases and clinical conditions associated with NEDD4L. The function of NEDD4L has been established by previous studies. Using cosmids from a human chromosome 18-specific library, Chen et al. (2001) used exon trapping and cDNA cloning to identify a gene homologous to NEDD4. The full-length cDNA sequence of 3,246 bp, obtained by RACE, contains an open reading frame of 2,562 nucleotides. The deduced 854-amino acid polypeptide was predicted to contain 4 WW domains and an HECT ubiquitin-protein ligase domain, highly conserved features in the NEDD4 gene family. The NEDD4L gene has 97% and 62% amino acid sequence identity to mouse Nedd4-2 and human NEDD4 genes, respectively. By expression analysis, a 3.4-kb band was observed in heart and muscle, while a 3.2-kb band and/or an additional 3.6-kb band were seen in other tissues examined. An alternative splicing event involving exon 12 of 60 bp was observed, the shorter allele being predominantly present in brain and lymphocytes, while the longer allele was strongly expressed in kidney and placenta. Since the NEDD4L gene mapped to the region of 18q21 showing linkage evidence for a susceptibility locus for bipolar affective disorder (OMIM Ref. No. 125480), Chen et al. (2001) screened the NEDD4L gene for mutations in 3 unrelated bipolar I probands and their parents, but no mutations were detected. Due to the potential role of NEDD4L in regulating the epithelial sodium channel (ENaC; OMIM Ref. No. 600228), Chen et al. (2001) proposed it a candidate gene for autosomal dominant orthostatic hypotensive disorder (OMIM Ref. No. 143850), mapped to 18q21. Erdeniz and Rothstein (2000) found that the ubiquitination domain of KIAA0439 shares homology with the S. cerevisiae Rsp5, a ubiquitin-protein ligase. They analyzed Rsp5 mutant strains and concluded that Rsp5 may be involved in the degradation of the single-stranded DNA-binding protein Rfa1, thereby linking ubiquitin-dependent protein degradation to the replication-recombination machinery.

Full details of the abovementioned studies are described in the following publications, the disclosure of which are hereby incorporated by reference:
Chen, H.; Ross, C. A.; Wang, N.; Huo, Y.; MacKinnon, D. F.; Potash, J. B.; Simpson, S. G.; McMahon, F. J.; DePaulo, J. R., Jr.; McInnis, M. G.: NEDD4L on human chromosome 18q21 has multiple forms of transcripts and is a homologue of the mouse Nedd_{—}4-2 gene. Europ. J. Hum. Genet. 9: 922-930, 2001.; and
Erdeniz, N.; Rothstein, R.: Rsp5, a ubiquitin-protein ligase, is involved in degradation of the single-stranded-DNA binding protein Rfa1 in Saccharomyces cerevisiae. Molec. Cell. Bio.

Further studies establishing the function and utilities of NEDD4L are found in John Hopkins OMIM database record ID 606384, and in sited publications numbered 1505-150 and 1712 listed in the bibliography section hereinbelow, which are also hereby incorporated by reference. Nuclear Receptor Sub family 3, Group C, Member 1 (glucocorticoid receptor) (NR3C1, Accession NM_{—}000176) is another VGAM97 host target gene. NR3C1 BINDING SITE is HOST TARGET binding site found in the 3′ untranslated region of mRNA encoded by NR3C1, corresponding to a HOST TARGET binding site such as BINDING SITE I, BINDING SITE II or BINDING SITE III. Table 2 illustrates the complementarity of the nucleotide sequences of NR3C1 BINDING SITE, designated SEQ ID:717, to the nucleotide sequence of VGAM97 RNA, herein designated VGAM RNA, also designated SEQ ID:432.

Another function of VGAM97 is therefore inhibition of Nuclear Receptor Sub family 3, Group C, Member 1 (glucocorticoid receptor) (NR3C1, Accession NM_{—}000176). Accordingly, utilities of VGAM97 include diagnosis, prevention and treatment of diseases and clinical conditions associated with NR3C1. Pyruvate Dehydrogenase Kinase, Isoenzyme 4 (PDK4, Accession XM_{—}173198) is another VGAM97 host target gene. PDK4 BINDING SITE is HOST TARGET binding site found in the 3′ untranslated region of mRNA encoded by PDK4, corresponding to a HOST TARGET binding site such as BINDING SITE I, BINDING SITE II or BINDING SITE III. Table 2 illustrates the complementarity of the nucleotide sequences of PDK4 BINDING SITE, designated SEQ ID:3733, to the nucleotide sequence of VGAM97 RNA, herein designated VGAM RNA, also designated SEQ ID:432.

Another function of VGAM97 is therefore inhibition of Pyruvate Dehydrogenase Kinase, Isoenzyme 4 (PDK4, Accession XM_{—}173198). Accordingly, utilities of VGAM97 include diagnosis, prevention and treatment of diseases and clinical conditions associated with PDK4. Presenilin 1 (Alzheimer disease 3) (PSEN1, Accession NM_{—}007319) is another VGAM97 host target gene. PSEN1 BINDING SITE is HOST TARGET binding site found in the 3′ untranslated region of mRNA encoded by PSEN1, corresponding to a HOST TARGET binding site such as BINDING SITE I, BINDING SITE II or BINDING SITE III. Table 2 illustrates the complementarity of the nucleotide sequences of PSEN1 BINDING SITE, designated SEQ ID:1393, to the nucleotide sequence of VGAM97 RNA, herein designated VGAM RNA, also designated SEQ ID:432.

Another function of VGAM97 is therefore inhibition of Presenilin 1 (Alzheimer disease 3) (PSEN1, Accession NM_{—}007319). Accordingly, utilities of VGAM97 include diagnosis, prevention and treatment of diseases and clinical conditions associated with PSEN1. Recombination Activating Gene 1 (RAG1, Accession NM_{—}000448) is another VGAM97 host target gene. RAG1 BINDING SITE is HOST TARGET binding site found in the 3′ untranslated region of mRNA encoded by RAG1, corresponding to a HOST TARGET binding site such as BINDING SITE I, BINDING SITE II or BINDING SITE III. Table 2 illustrates the complementarity of the nucleotide sequences of RAG1 BINDING SITE, designated SEQ ID:746, to the nucleotide sequence of VGAM97 RNA, herein designated VGAM RNA, also designated SEQ ID:432.

Another function of VGAM97 is therefore inhibition of Recombination Activating Gene 1 (RAG1, Accession NM_{—}000448). Accordingly, utilities of VGAM97 include diagnosis, prevention and treatment of diseases and clinical conditions associated with RAG1. Synaptogyrin 1 (SYNGR1, Accession NM_{—}004711) is another VGAM97 host target gene. SYNGR1 BINDING SITE is HOST TARGET binding site found in the 3′ untranslated region of mRNA encoded by SYNGR1, corresponding to a HOST TARGET binding site such as BINDING SITE I, BINDING SITE II or BINDING SITE III. Table 2 illustrates the complementarity of the nucleotide sequences of SYNGR1 BINDING SITE, designated SEQ ID:1148, to the nucleotide sequence of VGAM97 RNA, herein designated VGAM RNA, also designated SEQ ID:432.

Another function of VGAM97 is therefore inhibition of Synaptogyrin 1 (SYNGR1, Accession NM_{—}004711), a gene which belongs to transmembrane synaptic vesicle protein and may function in membrane recycling. Accordingly, utilities of VGAM97 include diagnosis, prevention and treatment of diseases and clinical conditions associated with SYNGR1. The function of SYNGR1 has been established by previous studies. Rat synaptogyrin, or RATSYNGR1, is an integral membrane protein associated with presynaptic vesicles in neuronal cells. See SYNGR2 (OMIM Ref. No. 603926). As part of an effort to sequence the long arm of human chromosome 22, Kedra et al. (1998) identified the human homolog of RATSYNGR1, synaptogyrin-1 (OMIM Ref. No. SYNGR1). By a combination of EST database searching and library screening, the authors isolated cDNAs corresponding to 3 alternatively spliced transcripts, which they designated SYNGR1a-c. The predicted 1a, 1b, and 1c proteins contain 234, 191, and 192 amino acids, respectively. Northern blot analysis revealed that the 4.5-kb SYNGR1a mRNA is expressed at high levels in brain. The other transcript forms are expressed at low levels in nonneuronal tissues. In situ hybridization to embryonic and adult mouse tissues confirmed that SYNGR1a, the most abundant transcript form, shows predominantly neuronal expression. Kedra et al. (1998) also identified cDNAs encoding the related human proteins SYNGR2 and SYNGR3 (OMIM Ref. No. 603927) and mouse Syngr1b. Like RATSYNGR1, the mouse and human synaptogyrin family members contain 4 membrane-spanning domains. The conserved central portion of SYNGR1a shares 54%, 61%, and 92% identity with that of SYNGR2, SYNGR3, and RATSYNGR1, respectively. Animal model experiments lend further support to the function of SYNGR1. Using gene targeting, Janz et al. (1999) generated mice lacking Syngr1. They bred these Syngr1 knockout mice against Syp (OMIM Ref. No. 313475) knockout mice generated by McMahon et al. (1996) to create double knockout mice deficient in both Syp and Syngr1. Both single and double knockout mice were viable and fertile. Morphologic and biochemical analysis showed that the architecture and composition of synapses were unaltered in the brains of Syngr1 single knockout and Syngr1/Syp double knockout mutant mice. Electrophysiologic recordings in the hippocampal CA1 region revealed that short- and long-term synaptic plasticity was severely reduced in the Syngr1/Syp double knockout mice without changes in the fundamental release apparatus, vesicle cycling, or release probability. Janz et al. (1999) concluded that Syngr1 and Syp perform essential and redundant functions in synaptic plasticity without being required for synaptic transmission as such.

It is appreciated that the abovementioned animal model for SYNGR1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Janz, R.; Sudhof, T. C.; Hammer, R. E.; Unni, V.; Siegelbaum, S. A.; Bolshakov, V. Y.: Essential roles in synaptic plasticity for synaptogyrin I and synaptophysin I. Neuron 24: 687-700, 1999.; and
Kedra, D.; Pan, H.-Q.; Seroussi, E.; Fransson, I.; Guilbaud, C.; Collins, J. E.; Dunham, I.; Blennow, E.; Roe, B. A.; Piehl, F.; Dumanski, J. P.: Characterization of the human synapto.

Further studies establishing the function and utilities of SYNGR1 are found in John Hopkins OMIM database record ID 603925, and in sited publications numbered 185 and 1858 listed in the bibliography section hereinbelow, which are also hereby incorporated by reference. TOX (Accession NM_{—}014729) is another VGAM97 host target gene. TOX BINDING SITE is HOST TARGET binding site found in the 5′ untranslated region of mRNA encoded by TOX, corresponding to a HOST TARGET binding site such as BINDING SITE I, BINDING SITE II or BINDING SITE III. Table 2 illustrates the complementarity of the nucleotide sequences of TOX BINDING SITE, designated SEQ ID:1534, to the nucleotide sequence of VGAM97 RNA, herein designated VGAM RNA, also designated SEQ ID:432.

Another function of VGAM97 is therefore inhibition of TOX (Accession NM_{—}014729). Accordingly, utilities of VGAM97 include diagnosis, prevention and treatment of diseases and clinical conditions associated with TOX. Transient Receptor Potential Cation Channel, Sub family C, Member 6 (TRPC6, Accession NM_{—}004621) is another VGAM97 host target gene. TRPC6 BINDING SITE is HOST TARGET binding site found in the 3′ untranslated region of mRNA encoded by TRPC6, corresponding to a HOST TARGET binding site such as BINDING SITE I, BINDING SITE II or BINDING SITE III. Table 2 illustrates the complementarity of the nucleotide sequences of TRPC6 BINDING SITE, designated SEQ ID:1135, to the nucleotide sequence of VGAM97 RNA, herein designated VGAM RNA, also designated SEQ ID:432.

Another function of VGAM97 is therefore inhibition of Transient Receptor Potential Cation Channel, Sub family C, Member 6 (TRPC6, Accession NM_{—}004621), a gene which has calcium channel activity. Accordingly, utilities of VGAM97 include diagnosis, prevention and treatment of diseases and clinical conditions associated with TRPC6. The function of TRPC6 has been established by previous studies. TRPCs, mammalian homologs of the Drosophila transient receptor potential (trp) protein, are ion channels that are thought to mediate capacitative calcium entry into the cell. Using a PCR-based strategy, Hofmann et al. (1999) isolated cDNAs encoding TRPC6, a novel member of the TRPC family. The predicted 931-amino acid protein shares 93% identity with mouse Trpc6. The authors found that TRPC6 is a nonselective cation channel that is activated by diacylglycerol (DAG) in a membrane-delimited fashion, independently of protein kinase C. Although TRPC3 (OMIM Ref. No. 602345), the closest structural relative of TRPC6, is activated in the same manner, human TRPC1 and mouse Trpc4 (OMIM Ref. No. 603651) and Trpc5 (OMIM Ref. No. 300334) were unresponsive to DAG. The authors suggested that TRPC3 and TRPC6 represent the first members of a new functional family of second-messenger-operated cation channels that are activated by DAG. Northern blot analysis revealed that TRPC6 is expressed primarily in placenta, lung, spleen, ovary, and small intestine. By FISH, D'Esposito et al. (1998) mapped the TRPC6 gene to chromosome 11q21-q22.

Full details of the abovementioned studies are described in the following publications, the disclosure of which are hereby incorporated by reference:
Hofmann, T.; Obukhov, A. G.; Schaefer, M.; Harteneck, C.; Gudermann, T.; Schultz, G.: Direct activation of human TRPC6 and TRPC3 channels by diacylglycerol. Nature 397: 259-263, 1999.; and
D'Esposito, M.; Strazzullo, M.; Cuccurese, M.; Spalluto, C.; Rocchi, M.; D'Urso, M.; Ciccodicola, A.: Identification and assignment of the human transient receptor potential channel 6 gene.

Further studies establishing the function and utilities of TRPC6 are found in John Hopkins OMIM database record ID 603652, and in sited publications numbered 1344-1345 listed in the bibliography section hereinbelow, which are also hereby incorporated by reference. AF9Q34 (Accession NM_{—}032552) is another VGAM97 host target gene. AF9Q34 BINDING SITE is HOST TARGET binding site found in the 3′ untranslated region of mRNA encoded by AF9Q34, corresponding to a HOST TARGET binding site such as BINDING SITE I, BINDING SITE II or BINDING SITE III. Table 2 illustrates the complementarity of the nucleotide sequences of AF9Q34 BINDING SITE, designated SEQ ID:2258, to the nucleotide sequence of VGAM97 RNA, herein designated VGAM RNA, also designated SEQ ID:432.

Another function of VGAM97 is therefore inhibition of AF9Q34 (Accession NM_{—}032552). Accordingly, utilities of VGAM97 include diagnosis, prevention and treatment of diseases and clinical conditions associated with AF9Q34. Complement Component 1, Q Subcomponent, Receptor 1 (C1QR1, Accession NM_{—}012072) is another VGAM97 host target gene. C1QR1 BINDING SITE is HOST TARGET binding site found in the 3′ untranslated region of mRNA encoded by C1QR1, corresponding to a HOST TARGET binding site such as BINDING SITE I, BINDING SITE II or BINDING SITE III. Table 2 illustrates the complementarity of the nucleotide sequences of C1QR1 BINDING SITE, designated SEQ ID: 1402, to the nucleotide sequence of VGAM97 RNA, herein designated VGAM RNA, also designated SEQ ID:432.

Another function of VGAM97 is therefore inhibition of Complement Component 1, Q Subcomponent, Receptor 1 (C1QR1, Accession NM_{—}012072). Accordingly, utilities of VGAM97 include diagnosis, prevention and treatment of diseases and clinical conditions associated with C1QR1. FLJ10193 (Accession NM_{—}018019) is another VGAM97 host target gene. FLJ10193 BINDING SITE is HOST TARGET binding site found in the 3′ untranslated region of mRNA encoded by FLJ10193, corresponding to a HOST TARGET binding site such as BINDING SITE I, BINDING SITE II or BINDING SITE III. Table 2 illustrates the complementarity of the nucleotide sequences of FLJ10193 BINDING SITE, designated SEQ ID:1775, to the nucleotide sequence of VGAM97 RNA, herein designated VGAM RNA, also designated SEQ ID:432.

Another function of VGAM97 is therefore inhibition of FLJ10193 (Accession NM_{—}018019). Accordingly, utilities of VGAM97 include diagnosis, prevention and treatment of diseases and clinical conditions associated with FLJ10193. FLJ10989 (Accession NM_{—}018292) is another VGAM97 host target gene. FLJ10989 BINDING SITE is HOST TARGET binding site found in the 3′ untranslated region of mRNA encoded by FLJ10989, corresponding to a HOST TARGET binding site such as BINDING SITE I, BINDING SITE II or BINDING SITE III. Table 2 illustrates the complementarity of the nucleotide sequences of FLJ10989 BINDING SITE, designated SEQ ID:1810, to the nucleotide sequence of VGAM97 RNA, herein designated VGAM RNA, also designated SEQ ID:432.

Another function of VGAM97 is therefore inhibition of FLJ10989 (Accession NM_{—}018292). Accordingly, utilities of VGAM97 include diagnosis, prevention and treatment of diseases and clinical conditions associated with FLJ10989. FLJ20700 (Accession NM_{—}017932) is another VGAM97 host target gene. FLJ20700 BINDING SITE is HOST TARGET binding site found in the 3′ untranslated region of mRNA encoded by FLJ20700, corresponding to a HOST TARGET binding site such as BINDING SITE I, BINDING SITE II or BINDING SITE III. Table 2 illustrates the complementarity of the nucleotide sequences of FLJ20700 BINDING SITE, designated SEQ ID:1767, to the nucleotide sequence of VGAM97 RNA, herein designated VGAM RNA, also designated SEQ ID:432.

Another function of VGAM97 is therefore inhibition of FLJ20700 (Accession NM_{—}017932). Accordingly, utilities of VGAM97 include diagnosis, prevention and treatment of diseases and clinical conditions associated with FLJ20700. FLJ31101 (Accession NM_{—}017964) is another VGAM97 host target gene. FLJ31101 BINDING SITE is HOST TARGET binding site found in the 3′ untranslated region of mRNA encoded by FLJ31101, corresponding to a HOST TARGET binding site such as BINDING SITE I, BINDING SITE II or BINDING SITE III. Table 2 illustrates the complementarity of the nucleotide sequences of FLJ31101 BINDING SITE, designated SEQ ID:1772, to the nucleotide sequence of VGAM97 RNA, herein designated VGAM RNA, also designated SEQ ID:432.

Another function of VGAM97 is therefore inhibition of FLJ31101 (Accession NM_{—}017964). Accordingly, utilities of VGAM97 include diagnosis, prevention and treatment of diseases and clinical conditions associated with FLJ31101. KIAA0252 (Accession XM_{—}031646) is another VGAM97 host target gene. KIAA0252 BINDING SITE is HOST TARGET binding site found in the 3′ untranslated region of mRNA encoded by KIAA0252, corresponding to a HOST TARGET binding site such as BINDING SITE I, BINDING SITE II or BINDING SITE III. Table 2 illustrates the complementarity of the nucleotide sequences of KIAA0252 BINDING SITE, designated SEQ ID:2638, to the nucleotide sequence of VGAM97 RNA, herein designated VGAM RNA, also designated SEQ ID:432.

Another function of VGAM97 is therefore inhibition of KIAA0252 (Accession XM_{—}031646). Accordingly, utilities of VGAM97 include diagnosis, prevention and treatment of diseases and clinical conditions associated with KIAA0252. KIAA0416 (Accession NM_{—}015564) is another VGAM97 host target gene. KIAA0416 BINDING SITE is HOST TARGET binding site found in the 3′ untranslated region of mRNA encoded by KIAA0416, corresponding to a HOST TARGET binding site such as BINDING SITE I, BINDING SITE II or BINDING SITE III. Table 2 illustrates the complementarity of the nucleotide sequences of KIAA0416 BINDING SITE, designated SEQ ID: 1638, to the nucleotide sequence of VGAM97 RNA, herein designated VGAM RNA, also designated SEQ ID:432.

Another function of VGAM97 is therefore inhibition of KIAA0416 (Accession NM_{—}015564). Accordingly, utilities of VGAM97 include diagnosis, prevention and treatment of diseases and clinical conditions associated with KIAA0416. KIAA0445 (Accession NM_{—}014675) is another VGAM97 host target gene. KIAA0445 BINDING SITE is HOST TARGET binding site found in the 5′ untranslated region of mRNA encoded by KIAA0445, corresponding to a HOST TARGET binding site such as BINDING SITE I, BINDING SITE II or BINDING SITE III. Table 2 illustrates the complementarity of the nucleotide sequences of KIAA0445 BINDING SITE, designated SEQ ID:1519, to the nucleotide sequence of VGAM97 RNA, herein designated VGAM RNA, also designated SEQ ID:432.

Another function of VGAM97 is therefore inhibition of KIAA0445 (Accession NM_{—}014675). Accordingly, utilities of VGAM97 include diagnosis, prevention and treatment of diseases and clinical conditions associated with KIAA0445. KIAA0493 (Accession XM_{—}034717) is another VGAM97 host target gene. KIAA0493 BINDING SITE is HOST TARGET binding site found in the 5′ untranslated region of mRNA encoded by KIAA0493, corresponding to a HOST TARGET binding site such as BINDING SITE I, BINDING SITE II or BINDING SITE III. Table 2 illustrates the complementarity of the nucleotide sequences of KIAA0493 BINDING SITE, designated SEQ ID:2683, to the nucleotide sequence of VGAM97 RNA, herein designated VGAM RNA, also designated SEQ ID:432.

Another function of VGAM97 is therefore inhibition of KIAA0493 (Accession XM_{—}034717). Accordingly, utilities of VGAM97 include diagnosis, prevention and treatment of diseases and clinical conditions associated with KIAA0493. KIAA0753 (Accession NM_{—}014804) is another VGAM97 host target gene. KIAA0753 BINDING SITE is HOST TARGET binding site found in the 3′ untranslated region of mRNA encoded by KIAA0753, corresponding to a HOST TARGET binding site such as BINDING SITE I, BINDING SITE II or BINDING SITE III. Table 2 illustrates the complementarity of the nucleotide sequences of KIAA0753 BINDING SITE, designated SEQ ID:1553, to the nucleotide sequence of VGAM97 RNA, herein designated VGAM RNA, also designated SEQ ID:432.

Another function of VGAM97 is therefore inhibition of KIAA0753 (Accession NM_{—}014804). Accordingly, utilities of VGAM97 include diagnosis, prevention and treatment of diseases and clinical conditions associated with KIAA0753. KIAA0781 (Accession XM_{—}041314) is another VGAM97 host target gene. KIAA0781 BINDING SITE is HOST TARGET binding site found in the 3′ untranslated region of mRNA encoded by KIAA0781, corresponding to a HOST TARGET binding site such as BINDING SITE I, BINDING SITE II or BINDING SITE III. Table 2 illustrates the complementarity of the nucleotide sequences of KIAA0781 BINDING SITE, designated SEQ ID:2794, to the nucleotide sequence of VGAM97 RNA, herein designated VGAM RNA, also designated SEQ ID:432.

Another function of VGAM97 is therefore inhibition of KIAA0781 (Accession XM_{—}041314). Accordingly, utilities of VGAM97 include diagnosis, prevention and treatment of diseases and clinical conditions associated with KIAA0781. KIAA0825 (Accession XM_{—}027906) is another VGAM97 host target gene. KIAA0825 BINDING SITE is HOST TARGET binding site found in the 3′ untranslated region of mRNA encoded by KIAA0825, corresponding to a HOST TARGET binding site such as BINDING SITE I, BINDING SITE II or BINDING SITE III. Table 2 illustrates the complementarity of the nucleotide sequences of KIAA0825 BINDING SITE, designated SEQ ID:2589, to the nucleotide sequence of VGAM97 RNA, herein designated VGAM RNA, also designated SEQ ID:432.

Another function of VGAM97 is therefore inhibition of KIAA0825 (Accession XM_{—}027906). Accordingly, utilities of VGAM97 include diagnosis, prevention and treatment of diseases and clinical conditions associated with KIAA0825. KIAA0831 (Accession NM_{—}014924) is another VGAM97 host target gene. KIAA0831 BINDING SITE is HOST TARGET binding site found in the 3′ untranslated region of mRNA encoded by KIAA0831, corresponding to a HOST TARGET binding site such as BINDING SITE I, BINDING SITE II or BINDING SITE III. Table 2 illustrates the complementarity of the nucleotide sequences of KIAA0831 BINDING SITE, designated SEQ ID:1590, to the nucleotide sequence of VGAM97 RNA, herein designated VGAM RNA, also designated SEQ ID:432.

Another function of VGAM97 is therefore inhibition of KIAA0831 (Accession NM_{—}014924). Accordingly, utilities of VGAM97 include diagnosis, prevention and treatment of diseases and clinical conditions associated with KIAA0831. KIAA1172 (Accession XM_{—}047889) is another VGAM97 host target gene. KIAA1172 BINDING SITE is HOST TARGET binding site found in the 3′ untranslated region of mRNA encoded by KIAA1172, corresponding to a HOST TARGET binding site such as BINDING SITE I, BINDING SITE II or BINDING SITE III. Table 2 illustrates the complementarity of the nucleotide sequences of KIAA1172 BINDING SITE, designated SEQ ID:2897, to the nucleotide sequence of VGAM97 RNA, herein designated VGAM RNA, also designated SEQ ID:432.

Another function of VGAM97 is therefore inhibition of KIAA1172 (Accession XM_{—}047889). Accordingly, utilities of VGAM97 include diagnosis, prevention and treatment of diseases and clinical conditions associated with KIAA1172. KR18 (Accession NM_{—}033288) is another VGAM97 host target gene. KR18 BINDING SITE is HOST TARGET binding site found in the 3′ untranslated region of mRNA encoded by KR18, corresponding to a HOST TARGET binding site such as BINDING SITE I, BINDING SITE II or BINDING SITE III. Table 2 illustrates the complementarity of the nucleotide sequences of KR18 BINDING SITE, designated SEQ ID:2325, to the nucleotide sequence of VGAM97 RNA, herein designated VGAM RNA, also designated SEQ ID:432.

Another function of VGAM97 is therefore inhibition of KR18 (Accession NM_{—}033288). Accordingly, utilities of VGAM97 include diagnosis, prevention and treatment of diseases and clinical conditions associated with KR18. Mitogen-activated Protein Kinase 13 (MAPK13, Accession NM_{—}002754) is another VGAM97 host target gene. MAPK13 BINDING SITE is HOST TARGET binding site found in the 3′ untranslated region of mRNA encoded by MAPK13, corresponding to a HOST TARGET binding site such as BINDING SITE I, BINDING SITE II or BINDING SITE III. Table 2 illustrates the complementarity of the nucleotide sequences of MAPK13 BINDING SITE, designated SEQ ID:953, to the nucleotide sequence of VGAM97 RNA, herein designated VGAM RNA, also designated SEQ ID:432.

Another function of VGAM97 is therefore inhibition of Mitogen-activated Protein Kinase 13 (MAPK13, Accession NM_{—}002754). Accordingly, utilities of VGAM97 include diagnosis, prevention and treatment of diseases and clinical conditions associated with MAPK13. MGC11386 (Accession NM_{—}032933) is another VGAM97 host target gene. MGC11386 BINDING SITE is HOST TARGET binding site found in the 3′ untranslated region of mRNA encoded by MGC11386, corresponding to a HOST TARGET binding site such as BINDING SITE I, BINDING SITE II or BINDING SITE III. Table 2 illustrates the complementarity of the nucleotide sequences of MGC11386 BINDING SITE, designated SEQ ID:2296, to the nucleotide sequence of VGAM97 RNA, herein designated VGAM RNA, also designated SEQ ID:432.

Another function of VGAM97 is therefore inhibition of MGC11386 (Accession NM_{—}032933). Accordingly, utilities of VGAM97 include diagnosis, prevention and treatment of diseases and clinical conditions associated with MGC11386. PRO0641 (Accession NM_{—}014135) is another VGAM97 host target gene. PRO0641 BINDING SITE is HOST TARGET binding site found in the 3′ untranslated region of mRNA encoded by PRO0641, corresponding to a HOST TARGET binding site such as BINDING SITE I, BINDING SITE II or BINDING SITE III. Table 2 illustrates the complementarity of the nucleotide sequences of PRO0641 BINDING SITE, designated SEQ ID: 1477, to the nucleotide sequence of VGAM97 RNA, herein designated VGAM RNA, also designated SEQ ID:432.

Another function of VGAM97 is therefore inhibition of PRO0641 (Accession NM_{—}014135). Accordingly, utilities of VGAM97 include diagnosis, prevention and treatment of diseases and clinical conditions associated with PRO0641. PRO0902 (Accession NM_{—}053057) is another VGAM97 host target gene. PRO0902 BINDING SITE is HOST TARGET binding site found in the 3′ untranslated region of mRNA encoded by PRO0902, corresponding to a HOST TARGET binding site such as BINDING SITE I, BINDING SITE II or BINDING SITE III. Table 2 illustrates the complementarity of the nucleotide sequences of PRO0902 BINDING SITE, designated SEQ ID:2358, to the nucleotide sequence of VGAM97 RNA, herein designated VGAM RNA, also designated SEQ ID:432.

Another function of VGAM97 is therefore inhibition of PRO0902 (Accession NM_{—}053057). Accordingly, utilities of VGAM97 include diagnosis, prevention and treatment of diseases and clinical conditions associated with PRO0902. SMAP-5 (Accession NM_{—}030799) is another VGAM97 host target gene. SMAP-5 BINDING SITE is HOST TARGET binding site found in the 3′ untranslated region of mRNA encoded by SMAP-5, corresponding to a HOST TARGET binding site such as BINDING SITE I, BINDING SITE II or BINDING SITE III. Table 2 illustrates the complementarity of the nucleotide sequences of SMAP-5 BINDING SITE, designated SEQ ID:2168, to the nucleotide sequence of VGAM97 RNA, herein designated VGAM RNA, also designated SEQ ID:432.

Another function of VGAM97 is therefore inhibition of SMAP-5 (Accession NM_{—}030799). Accordingly, utilities of VGAM97 include diagnosis, prevention and treatment of diseases and clinical conditions associated with SMAP-5. Sprouty Homolog 4 (Drosophila) (SPRY4, Accession NM_{—}030964) is another VGAM97 host target gene. SPRY4 BINDING SITE is HOST TARGET binding site found in the 3′ untranslated region of mRNA encoded by SPRY4, corresponding to a HOST TARGET binding site such as BINDING SITE I, BINDING SITE II or BINDING SITE III. Table 2 illustrates the complementarity of the nucleotide sequences of SPRY4 BINDING SITE, designated SEQ ID:2177, to the nucleotide sequence of VGAM97 RNA, herein designated VGAM RNA, also designated SEQ ID:432.

Another function of VGAM97 is therefore inhibition of Sprouty Homolog 4 (Drosophila) (SPRY4, Accession NM_{—}030964). Accordingly, utilities of VGAM97 include diagnosis, prevention and treatment of diseases and clinical conditions associated with SPRY4. Ubiquitin Specific Protease 24 (USP24, Accession XM_{—}165973) is another VGAM97 host target gene. USP24 BINDING SITE is HOST TARGET binding site found in the 3′ untranslated region of mRNA encoded by USP24, corresponding to a HOST TARGET binding site such as BINDING SITE I, BINDING SITE II or BINDING SITE III. Table 2 illustrates the complementarity of the nucleotide sequences of USP24 BINDING SITE, designated SEQ ID:3512, to the nucleotide sequence of VGAM97 RNA, herein designated VGAM RNA, also designated SEQ ID:432.

Another function of VGAM97 is therefore inhibition of Ubiquitin Specific Protease 24 (USP24, Accession XM_{—}165973). Accordingly, utilities of VGAM97 include diagnosis, prevention and treatment of diseases and clinical conditions associated with USP24. Zinc Finger Protein 304 (ZNF304, Accession NM_{—}020657) is another VGAM97 host target gene. ZNF304 BINDING SITE is HOST TARGET binding site found in the 3′ untranslated region of mRNA encoded by ZNF304, corresponding to a HOST TARGET binding site such as BINDING SITE I, BINDING SITE II or BINDING SITE III. Table 2 illustrates the complementarity of the nucleotide sequences of ZNF304 BINDING SITE, designated SEQ ID:1921, to the nucleotide sequence of VGAM97 RNA, herein designated VGAM RNA, also designated SEQ ID:432.

Another function of VGAM97 is therefore inhibition of Zinc Finger Protein 304 (ZNF304, Accession NM_{—}020657). Accordingly, utilities of VGAM97 include diagnosis, prevention and treatment of diseases and clinical conditions associated with ZNF304. LOC121344 (Accession XM_{—}058555) is another VGAM97 host target gene. LOC121344 BINDING SITE is HOST TARGET binding site found in the 3′ untranslated region of mRNA encoded by LOC121344, corresponding to a HOST TARGET binding site such as BINDING SITE I, BINDING SITE II or BINDING SITE III. Table 2 illustrates the complementarity of the nucleotide sequences of LOC121344 BINDING SITE, designated SEQ ID:2990, to the nucleotide sequence of VGAM97 RNA, herein designated VGAM RNA, also designated SEQ ID:432.

Another function of VGAM97 is therefore inhibition of LOC121344 (Accession XM_{—}058555). Accordingly, utilities of VGAM97 include diagnosis, prevention and treatment of diseases and clinical conditions associated with LOC121344. LOC144266 (Accession XM_{—}084795) is another VGAM97 host target gene. LOC144266 BINDING SITE is HOST TARGET binding site found in the 5′ untranslated region of mRNA encoded by LOC144266, corresponding to a HOST TARGET binding site such as BINDING SITE I, BINDING SITE II or BINDING SITE III. Table 2 illustrates the complementarity of the nucleotide sequences of LOC144266 BINDING SITE, designated SEQ ID:3065, to the nucleotide sequence of VGAM97 RNA, herein designated VGAM RNA, also designated SEQ ID:432.

Another function of VGAM97 is therefore inhibition of LOC144266 (Accession XM_{—}084795). Accordingly, utilities of VGAM97 include diagnosis, prevention and treatment of diseases and clinical conditions associated with LOC144266. LOC145241 (Accession XM_{—}031799) is another VGAM97 host target gene. LOC145241 BINDING SITE is HOST TARGET binding site found in the 3′ untranslated region of mRNA encoded by LOC145241, corresponding to a HOST TARGET binding site such as BINDING SITE I, BINDING SITE II or BINDING SITE III. Table 2 illustrates the complementarity of the nucleotide sequences of LOC145241 BINDING SITE, designated SEQ ID:2640, to the nucleotide sequence of VGAM97 RNA, herein designated VGAM RNA, also designated SEQ ID:432.

Another function of VGAM97 is therefore inhibition of LOC145241 (Accession XM_{—}031799). Accordingly, utilities of VGAM97 include diagnosis, prevention and treatment of diseases and clinical conditions associated with LOC145241. LOC148545 (Accession XM_{—}086226) is another VGAM97 host target gene. LOC148545 BINDING SITE is HOST TARGET binding site found in the 5′ untranslated region of mRNA encoded by LOC148545, corresponding to a HOST TARGET binding site such as BINDING SITE I, BINDING SITE II or BINDING SITE III. Table 2 illustrates the complementarity of the nucleotide sequences of LOC148545 BINDING SITE, designated SEQ ID:3120, to the nucleotide sequence of VGAM97 RNA, herein designated VGAM RNA, also designated SEQ ID:432.

Another function of VGAM97 is therefore inhibition of LOC148545 (Accession XM_{—}086226). Accordingly, utilities of VGAM97 include diagnosis, prevention and treatment of diseases and clinical conditions associated with LOC148545. LOC149073 (Accession XM_{—}097577) is another VGAM97 host target gene. LOC149073 BINDING SITE is HOST TARGET binding site found in the 5′ untranslated region of mRNA encoded by LOC149073, corresponding to a HOST TARGET binding site such as BINDING SITE I, BINDING SITE II or BINDING SITE III. Table 2 illustrates the complementarity of the nucleotide sequences of LOC149073 BINDING SITE, designated SEQ ID:3305, to the nucleotide sequence of VGAM97 RNA, herein designated VGAM RNA, also designated SEQ ID:432.

Another function of VGAM97 is therefore inhibition of LOC149073 (Accession XM_{—}097577). Accordingly, utilities of VGAM97 include diagnosis, prevention and treatment of diseases and clinical conditions associated with LOC149073. LOC149448 (Accession XM_{—}097642) is another VGAM97 host target gene. LOC149448 BINDING SITE is HOST TARGET binding site found in the 3′ untranslated region of mRNA encoded by LOC149448, corresponding to a HOST TARGET binding site such as BINDING SITE I, BINDING SITE II or BINDING SITE III. Table 2 illustrates the complementarity of the nucleotide sequences of LOC149448 BINDING SITE, designated SEQ ID:3309, to the nucleotide sequence of VGAM97 RNA, herein designated VGAM RNA, also designated SEQ ID:432.

Another function of VGAM97 is therefore inhibition of LOC149448 (Accession XM_{—}097642). Accordingly, utilities of VGAM97 include diagnosis, prevention and treatment of diseases and clinical conditions associated with LOC149448. LOC149579 (Accession XM_{—}048743) is another VGAM97 host target gene. LOC149579 BINDING SITE is HOST TARGET binding site found in the 5′ untranslated region of mRNA encoded by LOC149579, corresponding to a HOST TARGET binding site such as BINDING SITE I, BINDING SITE II or BINDING SITE III. Table 2 illustrates the complementarity of the nucleotide sequences of LOC149579 BINDING SITE, designated SEQ ID:2907, to the nucleotide sequence of VGAM97 RNA, herein designated VGAM RNA, also designated SEQ ID:432.

Another function of VGAM97 is therefore inhibition of LOC149579 (Accession XM_{—}048743). Accordingly, utilities of VGAM97 include diagnosis, prevention and treatment of diseases and clinical conditions associated with LOC149579. LOC152641 (Accession XM_{—}087497) is another VGAM97 host target gene. LOC152641 BINDING SITE is HOST TARGET binding site found in the 5′ untranslated region of mRNA encoded by LOC152641, corresponding to a HOST TARGET binding site such as BINDING SITE I, BINDING SITE II or BINDING SITE III. Table 2 illustrates the complementarity of the nucleotide sequences of LOC152641 BINDING SITE, designated SEQ ID:3173, to the nucleotide sequence of VGAM97 RNA, herein designated VGAM RNA, also designated SEQ ID:432.

Another function of VGAM97 is therefore inhibition of LOC152641 (Accession XM_{—}087497). Accordingly, utilities of VGAM97 include diagnosis, prevention and treatment of diseases and clinical conditions associated with LOC152641. LOC169966 (Accession XM_{—}093010) is another VGAM97 host target gene. LOC169966 BINDING SITE is HOST TARGET binding site found in the 3′ untranslated region of mRNA encoded by LOC169966, corresponding to a HOST TARGET binding site such as BINDING SITE I, BINDING SITE II or BINDING SITE III. Table 2 illustrates the complementarity of the nucleotide sequences of LOC169966 BINDING SITE, designated SEQ ID:3253, to the nucleotide sequence of VGAM97 RNA, herein designated VGAM RNA, also designated SEQ ID:432.

Another function of VGAM97 is therefore inhibition of LOC169966 (Accession XM_{—}093010). Accordingly, utilities of VGAM97 include diagnosis, prevention and treatment of diseases and clinical conditions associated with LOC169966. LOC222234 (Accession XM_{—}168558) is another VGAM97 host target gene. LOC222234 BINDING SITE is HOST TARGET binding site found in the 3′ untranslated region of mRNA encoded by LOC222234, corresponding to a HOST TARGET binding site such as BINDING SITE I, BINDING SITE II or BINDING SITE III. Table 2 illustrates the complementarity of the nucleotide sequences of LOC222234 BINDING SITE, designated SEQ ID:3647, to the nucleotide sequence of VGAM97 RNA, herein designated VGAM RNA, also designated SEQ ID:432.

Another function of VGAM97 is therefore inhibition of LOC222234 (Accession XM_{—}168558). Accordingly, utilities of VGAM97 include diagnosis, prevention and treatment of diseases and clinical conditions associated with LOC222234. LOC254058 (Accession XM_{—}173209) is another VGAM97 host target gene. LOC254058 BINDING SITE is HOST TARGET binding site found in the 3′ untranslated region of mRNA encoded by LOC254058, corresponding to a HOST TARGET binding site such as BINDING SITE I, BINDING SITE II or BINDING SITE III. Table 2 illustrates the complementarity of the nucleotide sequences of LOC254058 BINDING SITE, designated SEQ ID:3735, to the nucleotide sequence of VGAM97 RNA, herein designated VGAM RNA, also designated SEQ ID:432.

Another function of VGAM97 is therefore inhibition of LOC254058 (Accession XM_{—}173209). Accordingly, utilities of VGAM97 include diagnosis, prevention and treatment of diseases and clinical conditions associated with LOC254058. LOC51110 (Accession NM_{—}016027) is another VGAM97 host target gene. LOC51110 BINDING SITE is HOST TARGET binding site found in the 3′ untranslated region of mRNA encoded by LOC51110, corresponding to a HOST TARGET binding site such as BINDING SITE I, BINDING SITE II or BINDING SITE III. Table 2 illustrates the complementarity of the nucleotide sequences of LOC51110 BINDING SITE, designated SEQ ID:1657, to the nucleotide sequence of VGAM97 RNA, herein designated VGAM RNA, also designated SEQ ID:432.

Another function of VGAM97 is therefore inhibition of LOC51110 (Accession NM_{—}016027). Accordingly, utilities of VGAM97 include diagnosis, prevention and treatment of diseases and clinical conditions associated with LOC51110. LOC51292 (Accession NM_{—}016576) is another VGAM97 host target gene. LOC51292 BINDING SITE is HOST TARGET binding site found in the 3′ untranslated region of mRNA encoded by LOC51292, corresponding to a HOST TARGET binding site such as BINDING SITE I, BINDING SITE II or BINDING SITE III. Table 2 illustrates the complementarity of the nucleotide sequences of LOC51292 BINDING SITE, designated SEQ ID: 1698, to the nucleotide sequence of VGAM97 RNA, herein designated VGAM RNA, also designated SEQ ID:432.

Another function of VGAM97 is therefore inhibition of LOC51292 (Accession NM_{—}016576). Accordingly, utilities of VGAM97 include diagnosis, prevention and treatment of diseases and clinical conditions associated with LOC51292. LOC92573 (Accession XM_{—}045884) is another VGAM97 host target gene. LOC92573 BINDING SITE is HOST TARGET binding site found in the 5′ untranslated region of mRNA encoded by LOC92573, corresponding to a HOST TARGET binding site such as BINDING SITE I, BINDING SITE II or BINDING SITE III. Table 2 illustrates the complementarity of the nucleotide sequences of LOC92573 BINDING SITE, designated SEQ ID:2870, to the nucleotide sequence of VGAM97 RNA, herein designated VGAM RNA, also designated SEQ ID:432.

Another function of VGAM97 is therefore inhibition of LOC92573 (Accession XM_{—}045884). Accordingly, utilities of VGAM97 include diagnosis, prevention and treatment of diseases and clinical conditions associated with LOC92573. FIG. 1 further provides a conceptual description of a novel bioinformatically detected viral gene of the present invention, referred to here as Viral Genomic Address Messenger 98 (VGAM98) viral gene, which modulates expression of respective host target genes thereof, the function and utility of which host target genes is known in the art.

VGAM98 is a novel bioinformatically detected regulatory, non protein coding, viral micro RNA (miRNA) gene. The method by which VGAM98 was detected is described hereinabove with reference to FIGS. 1-8.

VGAM98 gene, herein designated VGAM GENE, is a viral gene contained in the genome of Vaccinia Virus. VGAM98 host target gene, herein designated VGAM HOST TARGET GENE, is a human gene contained in the human genome.

VGAM98 gene encodes a VGAM98 precursor RNA, herein designated VGAM PRECURSOR RNA. Similar to other miRNA genes, and unlike most ordinary genes, VGAM98 precursor RNA does not encode a protein. A nucleotide sequence identical or highly similar to the nucleotide sequence of VGAM98 precursor RNA is designated SEQ ID:84, and is provided hereinbelow with reference to the sequence listing part. Nucleotide sequence SEQ ID:84 is located at position 34330 relative to the genome of Vaccinia Virus.

VGAM98 precursor RNA folds onto itself, forming VGAM98 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM98 folded precursor RNA into VGAM98 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM98 RNA is designated SEQ ID:433, and is provided hereinbelow with reference to the sequence listing part.

VGAM98 host target gene, herein designated VGAM HOST TARGET GENE, encodes a corresponding messenger RNA, VGAM98 host target RNA, herein designated VGAM HOST TARGET RNA. VGAM98 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM98 RNA, herein designated VGAM RNA, binds complementarily to one or more host target binding sites located in untranslated regions of VGAM98 host target RNA, herein designated VGAM HOST TARGET RNA. This complementary binding is due to the fact that the nucleotide sequence of VGAM98 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM98 RNA, herein designated VGAM RNA, may have a different number of host target binding sites in untranslated regions of a VGAM98 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM98 RNA, herein designated VGAM RNA, to host target binding sites on VGAM98 host target RNA, herein designated VGAM HOST TARGET RNA, such as BINDING SITE I, BINDING SITE II and BINDING SITE III, inhibits translation of VGAM98 host target RNA into VGAM98 host target protein, herein designated VGAM HOST TARGET PROTEIN. VGAM host target protein is therefore outlined by a broken line.

It is appreciated that VGAM98 host target gene, herein designated VGAM HOST TARGET GENE, in fact represents a plurality of VGAM98 host target genes. The mRNA of each one of this plurality of VGAM98 host target genes comprises one or more host target binding sites, each having a nucleotide sequence which is at least partly complementary to VGAM98 RNA, herein designated VGAM RNA, and which when bound by VGAM98 RNA causes inhibition of translation of respective one or more VGAM98 host target proteins.

It is further appreciated by one skilled in the art that the mode of translational inhibition illustrated by FIG. 1 with specific reference to translational inhibition exerted by VGAM98 gene, herein designated VGAM GENE, on one or more VGAM98 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM98 is inhibition of expression of host target genes, as part of a novel viral mechanism of attacking a host. Accordingly, utilities of VGAM98 include diagnosis, prevention and treatment of viral infection by Vaccinia Virus. Specific functions, and accordingly utilities, of VGAM98 correlate with, and may be deduced from, the identity of the host target genes which VGAM98 binds and inhibits, and the function of these host target genes, as elaborated hereinbelow.

Nucleotide sequences of the VGAM98 precursor RNA, herein designated VGAM PRECURSOR RNA, and of the ‘diced’ VGAM98 RNA, herein designated VGAM RNA, and a schematic representation of the secondary folding of VGAM98 folded precursor RNA, herein designated VGAM FOLDED PRECURSOR RNA, of VGAM98 are further described hereinbelow with reference to Table 1.

Nucleotide sequences of host target binding sites, such as BINDING SITE-I, BINDING SITE-II and BINDING SITE-III of FIG. 1, found on VGAM98 host target RNA, and schematic representation of the complementarity of each of these host target binding sites to VGAM98 RNA, herein designated VGAM RNA, are described hereinbelow with reference to Table 2.

As mentioned hereinabove with reference to FIG. 1, a function of VGAM98 gene, herein designated VGAM is inhibition of expression of VGAM98 target genes. It is appreciated that specific functions, and accordingly utilities, of VGAM98 correlate with, and may be deduced from, the identity of the target genes which VGAM98 binds and inhibits, and the function of these target genes, as elaborated hereinbelow.

Arginine Vasopressin (neurophysin II, antidiuretic hormone, diabetes insipidus, neurohypophyseal) (AVP, Accession NM_{—}000490) is a VGAM98 host target gene. AVP BINDING SITE is HOST TARGET binding site found in the 5′ untranslated region of mRNA encoded by AVP, corresponding to a HOST TARGET binding site such as BINDING SITE I, BINDING SITE II or BINDING SITE III. Table 2 illustrates the complementarity of the nucleotide sequences of AVP BINDING SITE, designated SEQ ID:752, to the nucleotide sequence of VGAM98 RNA, herein designated VGAM RNA, also designated SEQ ID:433.

A function of VGAM98 is therefore inhibition of Arginine Vasopressin (neurophysin II, antidiuretic hormone, diabetes insipidus, neurohypophyseal) (AVP, Accession NM_{—}000490). Accordingly, utilities of VGAM98 include diagnosis, prevention and treatment of diseases and clinical conditions associated with AVP. HTRA3 (Accession XM_{—}114416) is another VGAM98 host target gene. HTRA3 BINDING SITE is HOST TARGET binding site found in the 3′ untranslated region of mRNA encoded by HTRA3, corresponding to a HOST TARGET binding site such as BINDING SITE I, BINDING SITE II or BINDING SITE III. Table 2 illustrates the complementarity of the nucleotide sequences of HTRA3 BINDING SITE, designated SEQ ID:3457, to the nucleotide sequence of VGAM98 RNA, herein designated VGAM RNA, also designated SEQ ID:433.

Another function of VGAM98 is therefore inhibition of HTRA3 (Accession XM_{—}114416). Accordingly, utilities of VGAM98 include diagnosis, prevention and treatment of diseases and clinical conditions associated with HTRA3. Lectin, Galactoside-binding, Soluble, 3 Binding Protein (LGALS3BP, Accession XM_{—}045104) is another VGAM98 host target gene. LGALS3BP BINDING SITE is HOST TARGET binding site found in the 3′ untranslated region of mRNA encoded by LGALS3BP, corresponding to a HOST TARGET binding site such as BINDING SITE I, BINDING SITE II or BINDING SITE III. Table 2 illustrates the complementarity of the nucleotide sequences of LGALS3BP BINDING SITE, designated SEQ ID:2851, to the nucleotide sequence of VGAM98 RNA, herein designated VGAM RNA, also designated SEQ ID:433.

Another function of VGAM98 is therefore inhibition of Lectin, Galactoside-binding, Soluble, 3 Binding Protein (LGALS3BP, Accession XM_{—}045104). Accordingly, utilities of VGAM98 include diagnosis, prevention and treatment of diseases and clinical conditions associated with LGALS3BP. MAD2 Mitotic Arrest Deficient-like 1 (yeast) (MAD2Li, Accession NM_{—}002358) is another VGAM98 host target gene. MAD2L1 BINDING SITE is HOST TARGET binding site found in the 3′ untranslated region of mRNA encoded by MAD2Li, corresponding to a HOST TARGET binding site such as BINDING SITE I, BINDING SITE II or BINDING SITE III. Table 2 illustrates the complementarity of the nucleotide sequences of MAD2L1 BINDING SITE, designated SEQ ID:921, to the nucleotide sequence of VGAM98 RNA, herein designated VGAM RNA, also designated SEQ ID:433.

Another function of VGAM98 is therefore inhibition of MAD2 Mitotic Arrest Deficient-like 1 (yeast) (MAD2Li, Accession NM_{—}002358), a gene which may monitor the completeness of the spindle-kinetochore attachment. delays the onset of anaphase when this process is not complete. Accordingly, utilities of VGAM98 include diagnosis, prevention and treatment of diseases and clinical conditions associated with MAD2L1. The function of MAD2L1 has been established by previous studies. Using a yeast 2-hybrid analysis with the cytoplasmic tails of several a disintegrin and metalloproteinase domain (ADAM) proteins as bait, Nelson et al. (1999) found that MAD2L1 interacts strongly with TACE (ADAM17; 603639) but not with ADAM9 (OMIM Ref. No. 602713), which interacts with MAD2L2, or with other ADAMs tested. Further binding analyses defined a 35-amino acid stretch of TACE containing a proline-rich SH3-ligand domain (OMIM Ref. No. PXPXXP) as the interaction site for MAD2L1. Luo et al. (2002) showed that RNA interference-mediated suppression of MAD1 (OMIM Ref. No. 602686) function in mammalian cells caused loss of MAD2 kinetochore localization and impairment of the spindle checkpoint. MAD1 and CDC20 (OMIM Ref. No. 603618) contain MAD2-binding motifs that share a common consensus, and the authors identified a class of MAD2-binding peptides (MBPs) with a similar consensus. Binding of one of these ligands, MBP1, triggered an extensive rearrangement of the tertiary structure of MAD2. MAD2 also underwent a similar striking structural change upon binding to a MAD1 or CDC20 binding motif peptide. These data suggested that, upon checkpoint activation, MAD1 recruits MAD2 to unattached kinetochores and may promote binding of MAD2 to CDC20 Animal model experiments lend further support to the function of MAD2L1. The initiation of chromosome segregation at anaphase is linked by the spindle assembly checkpoint to the completion of chromosome-microtubule attachment during metaphase. To determine the function of the Mad2 protein during normal cell division, Dobles et al. (2000) knocked out the Mad2 gene in mice. They found that embryonic cells lacking Mad2 at embryonic day 5.5, like mad2 yeast, grew normally but were unable to arrest in response to spindle disruption. At embryonic day 6.5, the cells of the epiblast began rapid cell division, and the absence of a checkpoint resulted in widespread chromosome missegregation and apoptosis. In contrast, the postmitotic trophoblast giant cells survived without Mad2. Thus, the spindle assembly checkpoint is required for accurate chromosome segregation in mitotic mouse cells and for embryonic viability, even in the absence of spindle damage. Shonn et al. (2000) characterized the spindle checkpoint in meiosis of S. cerevisiae by comparing wildtype and mad2-deficient yeast. In the absence of the checkpoint, the frequency of meiosis I missegregation increased with increasing chromosome length, reaching 19% for the longest chromosome. Meiosis I nondisjunction in spindle checkpoint mutants could be prevented by delaying the onset of anaphase. In a recombinant-defective mutant, the checkpoint delayed the biochemical events of anaphase I, suggesting that chromosomes that are attached to microtubules but are not under tension can activate the spindle checkpoint. Spindle checkpoint mutants reduced the accuracy of chromosome segregation in meiosis I much more than that in meiosis II, suggesting that checkpoint defects may contribute to Down syndrome (OMIM Ref. No. 190685). Shonn et al. (2000) showed that the budding yeast spindle checkpoint, which is largely dispensable in wildtype mitosis, plays a critical role in meiotic chromosome segregation. They suggested that the difference may reflect the different chromosome linkages in mitosis and meiosis I. In mitosis, sister chromatid cohesion forces sister kinetochores to face opposite spindle poles. In meiosis I, homologs are linked at sites of recombination that can be far from the kinetochores, creating a floppy linkage. If the nearest recombination event is further from the centromere on long chromosomes, this idea may explain why long chromosomes preferentially nondisjoin in checkpoint-defective cells. Michel et al. (2001) reported that deletion of one MAD2 allele results in a defective mitotic checkpoint in both human cancer cells and murine primary embryonic fibroblasts. Checkpoint-defective cells show premature sister chromatid separation in the presence of spindle inhibitors and an elevated rate of chromosome missegregation events in the absence of these agents. Furthermore, Mad2+/−mice develop lung tumors at high rates after long latencies, implicating defects in the mitotic checkpoint in tumorigenesis

It is appreciated that the abovementioned animal model for MAD2L1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Michel, L. S.; Liberal, V.; Chatterjee, A.; Kirchwegger, R.; Pasche, B.; Gerald, W.; Dobles, M.; Sorger, P. K.; Murty, V. V. V. S.; Benezra, R.: MAD2 haplo-insufficiency causes premature anaphase and chromosome instability in mammalian cells. Nature 409: 355-359, 2001.; and
Luo, X.; Tang, Z.; Rizo, J.; Yu, H.: The Mad2 spindle checkpoint protein undergoes similar major conformational changes upon binding to either Mad1 or Cdc20. Molec. Cell 9: 59-71, 2002.

Further studies establishing the function and utilities of MAD2L1 are found in John Hopkins OMIM database record ID 601467, and in sited publications numbered 631-641 listed in the bibliography section hereinbelow, which are also hereby incorporated by reference. Phosphorylase, Glycogen; Muscle (McArdle syndrome, glycogen storage disease type V) (PYGM, Accession NM_{—}005609) is another VGAM98 host target gene. PYGM BINDING SITE is HOST TARGET binding site found in the 5′ untranslated region of mRNA encoded by PYGM, corresponding to a HOST TARGET binding site such as BINDING SITE I, BINDING SITE II or BINDING SITE III. Table 2 illustrates the complementarity of the nucleotide sequences of PYGM BINDING SITE, designated SEQ ID:1230, to the nucleotide sequence of VGAM98 RNA, herein designated VGAM RNA, also designated SEQ ID:433.

Another function of VGAM98 is therefore inhibition of Phosphorylase, Glycogen; Muscle (McArdle syndrome, glycogen storage disease type V) (PYGM, Accession NM_{—}005609). Accordingly, utilities of VGAM98 include diagnosis, prevention and treatment of diseases and clinical conditions associated with PYGM. Solute Carrier Family 35 (UDP-N-acetylglucosamine (UDP-GlcNAc) Transporter), Member 3 (SLC35A3, Accession NM_{—}012243) is another VGAM98 host target gene. SLC35A3 BINDING SITE is HOST TARGET binding site found in the 3′ untranslated region of mRNA encoded by SLC35A3, corresponding to a HOST TARGET binding site such as BINDING SITE I, BINDING SITE II or BINDING SITE III. Table 2 illustrates the complementarity of the nucleotide sequences of SLC35A3 BINDING SITE, designated SEQ ID:1418, to the nucleotide sequence of VGAM98 RNA, herein designated VGAM RNA, also designated SEQ ID:433.

Another function of VGAM98 is therefore inhibition of Solute Carrier Family 35 (UDP-N-acetylglucosamine (UDP-GlcNAc) Transporter), Member 3 (SLC35A3, Accession NM_{—}012243). Accordingly, utilities of VGAM98 include diagnosis, prevention and treatment of diseases and clinical conditions associated with SLC35A3. ATP Synthase Mitochondrial F1 Complex Assembly Factor 1 (ATPAF1, Accession XM_{—}027313) is another VGAM98 host target gene. ATPAF1 BINDING SITE is HOST TARGET binding site found in the 3′ untranslated region of mRNA encoded by ATPAF1, corresponding to a HOST TARGET binding site such as BINDING SITE I, BINDING SITE II or BINDING SITE III. Table 2 illustrates the complementarity of the nucleotide sequences of ATPAF1 BINDING SITE, designated SEQ ID:2583, to the nucleotide sequence of VGAM98 RNA, herein designated VGAM RNA, also designated SEQ ID:433.

Another function of VGAM98 is therefore inhibition of ATP Synthase Mitochondrial F1 Complex Assembly Factor 1 (ATPAF1, Accession XM_{—}027313). Accordingly, utilities of VGAM98 include diagnosis, prevention and treatment of diseases and clinical conditions associated with ATPAF1. Chromosome 7 Open Reading Frame 13 (C7orf13, Accession NM_{—}032625) is another VGAM98 host target gene. C7orf13 BINDING SITE is HOST TARGET binding site found in the 3′ untranslated region of mRNA encoded by C7orf13, corresponding to a HOST TARGET binding site such as BINDING SITE I, BINDING SITE II or BINDING SITE III. Table 2 illustrates the complementarity of the nucleotide sequences of C7orf13 BINDING SITE, designated SEQ ID:2265, to the nucleotide sequence of VGAM98 RNA, herein designated VGAM RNA, also designated SEQ ID:433.

Another function of VGAM98 is therefore inhibition of Chromosome 7 Open Reading Frame 13 (C7orf13, Accession NM_{—}032625). Accordingly, utilities of VGAM98 include diagnosis, prevention and treatment of diseases and clinical conditions associated with C7orf13. Cas-Br-M (murine) Ecotropic Retroviral Transforming Sequence C (CBLC, Accession NM_{—}012116) is another VGAM98 host target gene. CBLC BINDING SITE is HOST TARGET binding site found in the 3′ untranslated region of mRNA encoded by CBLC, corresponding to a HOST TARGET binding site such as BINDING SITE I, BINDING SITE II or BINDING SITE III. Table 2 illustrates the complementarity of the nucleotide sequences of CBLC BINDING SITE, designated SEQ ID: 1407, to the nucleotide sequence of VGAM98 RNA, herein designated VGAM RNA, also designated SEQ ID:433.

Another function of VGAM98 is therefore inhibition of Cas-Br-M (murine) Ecotropic Retroviral Transforming Sequence C (CBLC, Accession NM_{—}012116). Accordingly, utilities of VGAM98 include diagnosis, prevention and treatment of diseases and clinical conditions associated with CBLC. FLJ10241 (Accession NM_{—}018035) is another VGAM98 host target gene. FLJ10241 BINDING SITE is HOST TARGET binding site found in the 3′ untranslated region of mRNA encoded by FLJ10241, corresponding to a HOST TARGET binding site such as BINDING SITE I, BINDING SITE II or BINDING SITE III. Table 2 illustrates the complementarity of the nucleotide sequences of FLJ10241 BINDING SITE, designated SEQ ID:1776, to the nucleotide sequence of VGAM98 RNA, herein designated VGAM RNA, also designated SEQ ID:433.

Another function of VGAM98 is therefore inhibition of FLJ10241 (Accession NM_{—}018035). Accordingly, utilities of VGAM98 include diagnosis, prevention and treatment of diseases and clinical conditions associated with FLJ10241. FLJ20651 (Accession NM_{—}017919) is another VGAM98 host target gene. FLJ20651 BINDING SITE is HOST TARGET binding site found in the 3′ untranslated region of mRNA encoded by FLJ20651, corresponding to a HOST TARGET binding site such as BINDING SITE I, BINDING SITE II or BINDING SITE III. Table 2 illustrates the complementarity of the nucleotide sequences of FLJ20651 BINDING SITE, designated SEQ ID:1761, to the nucleotide sequence of VGAM98 RNA, herein designated VGAM RNA, also designated SEQ ID:433.

Another function of VGAM98 is therefore inhibition of FLJ20651 (Accession NM_{—}017919). Accordingly, utilities of VGAM98 include diagnosis, prevention and treatment of diseases and clinical conditions associated with FLJ20651. KIAA1203 (Accession XM_{—}049683) is another VGAM98 host target gene. KIAA1203 BINDING SITE is HOST TARGET binding site found in the 3′ untranslated region of mRNA encoded by KIAA1203, corresponding to a HOST TARGET binding site such as BINDING SITE I, BINDING SITE II or BINDING SITE III. Table 2 illustrates the complementarity of the nucleotide sequences of KIAA1203 BINDING SITE, designated SEQ ID:2922, to the nucleotide sequence of VGAM98 RNA, herein designated VGAM RNA, also designated SEQ ID:433.

Another function of VGAM98 is therefore inhibition of KIAA1203 (Accession XM_{—}049683). Accordingly, utilities of VGAM98 include diagnosis, prevention and treatment of diseases and clinical conditions associated with KIAA1203. PL6 (Accession NM_{—}007024) is another VGAM98 host target gene. PL6 BINDING SITE is HOST TARGET binding site found in the 3′ untranslated region of mRNA encoded by PL6, corresponding to a HOST TARGET binding site such as BINDING SITE I, BINDING SITE II or BINDING SITE III. Table 2 illustrates the complementarity of the nucleotide sequences of PL6 BINDING SITE, designated SEQ ID:1348, to the nucleotide sequence of VGAM98 RNA, herein designated VGAM RNA, also designated SEQ ID:433.

Another function of VGAM98 is therefore inhibition of PL6 (Accession NM_{—}007024). Accordingly, utilities of VGAM98 include diagnosis, prevention and treatment of diseases and clinical conditions associated with PL6. Scavenger Receptor Cysteine Rich Domain Containing, Group B (4 domains) (SRCRB4D, Accession NM_{—}080744) is another VGAM98 host target gene. SRCRB4D BINDING SITE is HOST TARGET binding site found in the 3′ untranslated region of mRNA encoded by SRCRB4D, corresponding to a HOST TARGET binding site such as BINDING SITE I, BINDING SITE II or BINDING SITE III. Table 2 illustrates the complementarity of the nucleotide sequences of SRCRB4D BINDING SITE, designated SEQ ID:2386, to the nucleotide sequence of VGAM98 RNA, herein designated VGAM RNA, also designated SEQ ID:433.

Another function of VGAM98 is therefore inhibition of Scavenger Receptor Cysteine Rich Domain Containing, Group B (4 domains) (SRCRB4D, Accession NM_{—}080744). Accordingly, utilities of VGAM98 include diagnosis, prevention and treatment of diseases and clinical conditions associated with SRCRB4D. Synaptopodin 2 (SYNPO2, Accession XM_{—}050219) is another VGAM98 host target gene. SYNPO2 BINDING SITE is HOST TARGET binding site found in the 3′ untranslated region of mRNA encoded by SYNPO2, corresponding to a HOST TARGET binding site such as BINDING SITE I, BINDING SITE II or BINDING SITE III. Table 2 illustrates the complementarity of the nucleotide sequences of SYNPO2 BINDING SITE, designated SEQ ID:2928, to the nucleotide sequence of VGAM98 RNA, herein designated VGAM RNA, also designated SEQ ID:433.

Another function of VGAM98 is therefore inhibition of Synaptopodin 2 (SYNPO2, Accession XM_{—}050219). Accordingly, utilities of VGAM98 include diagnosis, prevention and treatment of diseases and clinical conditions associated with SYNPO2. LOC145858 (Accession XM_{—}085258) is another VGAM98 host target gene. LOC145858 BINDING SITE is HOST TARGET binding site found in the 5′ untranslated region of mRNA encoded by LOC145858, corresponding to a HOST TARGET binding site such as BINDING SITE I, BINDING SITE II or BINDING SITE III. Table 2 illustrates the complementarity of the nucleotide sequences of LOC145858 BINDING SITE, designated SEQ ID:3085, to the nucleotide sequence of VGAM98 RNA, herein designated VGAM RNA, also designated SEQ ID:433.

Another function of VGAM98 is therefore inhibition of LOC145858 (Accession XM_{—}085258). Accordingly, utilities of VGAM98 include diagnosis, prevention and treatment of diseases and clinical conditions associated with LOC145858. LOC151162 (Accession XM_{—}098012) is another VGAM98 host target gene. LOC151162 BINDING SITE is HOST TARGET binding site found in the 5′ untranslated region of mRNA encoded by LOC151162, corresponding to a HOST TARGET binding site such as BINDING SITE I, BINDING SITE II or BINDING SITE III. Table 2 illustrates the complementarity of the nucleotide sequences of LOC151162 BINDING SITE, designated SEQ ID:3338, to the nucleotide sequence of VGAM98 RNA, herein designated VGAM RNA, also designated SEQ ID:433.

Another function of VGAM98 is therefore inhibition of LOC151162 (Accession XM_{—}098012). Accordingly, utilities of VGAM98 include diagnosis, prevention and treatment of diseases and clinical conditions associated with LOC151162. LOC196410 (Accession XM_{—}113713) is another VGAM98 host target gene. LOC196410 BINDING SITE is HOST TARGET binding site found in the 5′ untranslated region of mRNA encoded by LOC196410, corresponding to a HOST TARGET binding site such as BINDING SITE I, BINDING SITE II or BINDING SITE III. Table 2 illustrates the complementarity of the nucleotide sequences of LOC196410 BINDING SITE, designated SEQ ID:3411, to the nucleotide sequence of VGAM98 RNA, herein designated VGAM RNA, also designated SEQ ID:433.

Another function of VGAM98 is therefore inhibition of LOC196410 (Accession XM_{—}113713). Accordingly, utilities of VGAM98 include diagnosis, prevention and treatment of diseases and clinical conditions associated with LOC196410. LOC203339 (Accession XM_{—}117534) is another VGAM98 host target gene. LOC203339 BINDING SITE is HOST TARGET binding site found in the 5′ untranslated region of mRNA encoded by LOC203339, corresponding to a HOST TARGET binding site such as BINDING SITE I, BINDING SITE II or BINDING SITE III. Table 2 illustrates the complementarity of the nucleotide sequences of LOC203339 BINDING SITE, designated SEQ ID:3489, to the nucleotide sequence of VGAM98 RNA, herein designated VGAM RNA, also designated SEQ ID:433.

Another function of VGAM98 is therefore inhibition of LOC203339 (Accession XM_{—}117534). Accordingly, utilities of VGAM98 include diagnosis, prevention and treatment of diseases and clinical conditions associated with LOC203339. LOC203377 (Accession XM_{—}117540) is another VGAM98 host target gene. LOC203377 BINDING SITE is HOST TARGET binding site found in the 5′ untranslated region of mRNA encoded by LOC203377, corresponding to a HOST TARGET binding site such as BINDING SITE I, BINDING SITE II or BINDING SITE III. Table 2 illustrates the complementarity of the nucleotide sequences of LOC203377 BINDING SITE, designated SEQ ID:3490, to the nucleotide sequence of VGAM98 RNA, herein designated VGAM RNA, also designated SEQ ID:433.

Another function of VGAM98 is therefore inhibition of LOC203377 (Accession XM_{—}117540). Accordingly, utilities of VGAM98 include diagnosis, prevention and treatment of diseases and clinical conditions associated with LOC203377. FIG. 1 further provides a conceptual description of a novel bioinformatically detected viral gene of the present invention, referred to here as Viral Genomic Address Messenger 99 (VGAM99) viral gene, which modulates expression of respective host target genes thereof, the function and utility of which host target genes is known in the art.

VGAM99 is a novel bioinformatically detected regulatory, non protein coding, viral micro RNA (miRNA) gene. The method by which VGAM99 was detected is described hereinabove with reference to FIGS. 1-8.

VGAM99 gene, herein designated VGAM GENE, is a viral gene contained in the genome of Vaccinia Virus. VGAM99 host target gene, herein designated VGAM HOST TARGET GENE, is a human gene contained in the human genome.

VGAM99 gene encodes a VGAM99 precursor RNA, herein designated VGAM PRECURSOR RNA. Similar to other miRNA genes, and unlike most ordinary genes, VGAM99 precursor RNA does not encode a protein. A nucleotide sequence identical or highly similar to the nucleotide sequence of VGAM99 precursor RNA is designated SEQ ID:85, and is provided hereinbelow with reference to the sequence listing part. Nucleotide sequence SEQ ID:85 is located at position 39758 relative to the genome of Vaccinia Virus.

VGAM99 precursor RNA folds onto itself, forming VGAM99 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM99 folded precursor RNA into VGAM99 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM99 RNA is designated SEQ ID:434, and is provided hereinbelow with reference to the sequence listing part.

VGAM99 host target gene, herein designated VGAM HOST TARGET GENE, encodes a corresponding messenger RNA, VGAM99 host target RNA, herein designated VGAM HOST TARGET RNA. VGAM99 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM99 RNA, herein designated VGAM RNA, binds complementarily to one or more host target binding sites located in untranslated regions of VGAM99 host target RNA, herein designated VGAM HOST TARGET RNA. This complementary binding is due to the fact that the nucleotide sequence of VGAM99 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM99 RNA, herein designated VGAM RNA, may have a different number of host target binding sites in untranslated regions of a VGAM99 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM99 RNA, herein designated VGAM RNA, to host target binding sites on VGAM99 host target RNA, herein designated VGAM HOST TARGET RNA, such as BINDING SITE I, BINDING SITE II and BINDING SITE III, inhibits translation of VGAM99 host target RNA into VGAM99 host target protein, herein designated VGAM HOST TARGET PROTEIN. VGAM host target protein is therefore outlined by a broken line.

It is appreciated that VGAM99 host target gene, herein designated VGAM HOST TARGET GENE, in fact represents a plurality of VGAM99 host target genes. The mRNA of each one of this plurality of VGAM99 host target genes comprises one or more host target binding sites, each having a nucleotide sequence which is at least partly complementary to VGAM99 RNA, herein designated VGAM RNA, and which when bound by VGAM99 RNA causes inhibition of translation of respective one or more VGAM99 host target proteins.

It is further appreciated by one skilled in the art that the mode of translational inhibition illustrated by FIG. 1 with specific reference to translational inhibition exerted by VGAM99 gene, herein designated VGAM GENE, on one or more VGAM99 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM99 is inhibition of expression of host target genes, as part of a novel viral mechanism of attacking a host. Accordingly, utilities of VGAM99 include diagnosis, prevention and treatment of viral infection by Vaccinia Virus. Specific functions, and accordingly utilities, of VGAM99 correlate with, and may be deduced from, the identity of the host target genes which VGAM99 binds and inhibits, and the function of these host target genes, as elaborated hereinbelow.

Nucleotide sequences of the VGAM99 precursor RNA, herein designated VGAM PRECURSOR RNA, and of the ‘diced’ VGAM99 RNA, herein designated VGAM RNA, and a schematic representation of the secondary folding of VGAM99 folded precursor RNA, herein designated VGAM FOLDED PRECURSOR RNA, of VGAM99 are further described hereinbelow with reference to Table 1.

Nucleotide sequences of host target binding sites, such as BINDING SITE-I, BINDING SITE-II and BINDING SITE-III of FIG. 1, found on VGAM99 host target RNA, and schematic representation of the complementarity of each of these host target binding sites to VGAM99 RNA, herein designated VGAM RNA, are described hereinbelow with reference to Table 2.

As mentioned hereinabove with reference to FIG. 1, a function of VGAM99 gene, herein designated VGAM is inhibition of expression of VGAM99 target genes. It is appreciated that specific functions, and accordingly utilities, of VGAM99 correlate with, and may be deduced from, the identity of the target genes which VGAM99 binds and inhibits, and the function of these target genes, as elaborated hereinbelow.

Sel-1 Suppressor of Lin-12-like (C. elegans) (SEL1L, Accession NM_{—}005065) is a VGAM99 host target gene. SEL1L BINDING SITE is HOST TARGET binding site found in the 3′ untranslated region of mRNA encoded by SEL1L, corresponding to a HOST TARGET binding site such as BINDING SITE I, BINDING SITE II or BINDING SITE III. Table 2 illustrates the complementarity of the nucleotide sequences of SEL1L BINDING SITE, designated SEQ ID:1179, to the nucleotide sequence of VGAM99 RNA, herein designated VGAM RNA, also designated SEQ ID:434.

A function of VGAM99 is therefore inhibition of Sel-1 Suppressor of Lin-12-like (C. elegans) (SEL1L, Accession NM_{—}005065), a gene which may play a role in notch signaling (by similarity). Accordingly, utilities of VGAM99 include diagnosis, prevention and treatment of diseases and clinical conditions associated with SEL1L. The function of SEL1L has been established by previous studies. Biunno et al. (1997) isolated a novel cDNA, designated SEL1L by them, that shows sequence similarities to sel-1, a gene identified as an extragenic suppressor of the lin-12 hypomorphic mutant from C. elegans (3,4: Grant and Greenwald, 1996, 1997). SEL1L exhibited a tissue-specific pattern of expression: high levels of a single 7.5-kb transcript were detected only in the pancreas of healthy individuals, whereas low to undetectable levels were observed in other adult tissues and in some fetal tissues. Because of the tissue-specific expression of the gene, Biunno et al. (1997) studied the gene in human pancreatic carcinomas. They found that 17% of adenocarcinomas of the pancreas did not express SEL1L to a detectable level; however, no gross genomic alterations were apparent within a few hundred kb of the relevant region. By somatic cell hybrid analysis and fluorescence in situ hybridization, Biunno et al. (1997) mapped the SEL1L gene to chromosome 14q31. Donoviel and Bernstein (1999) localized the gene to 14q24.3-q31 by FISH and radiation hybrid analysis.

Full details of the abovementioned studies are described in the following publications, the disclosure of which are hereby incorporated by reference:
Biunno, I.; Appierto, V.; Cattaneo, M.; Leone, B. E.; Balzano, G.; Socci, C.; Saccone, S.; Letizia, A.; Valle, G. D.; Sgaramella, V.: Isolation of a pancreas-specific gene located on human chromosome 14q31: expression analysis in human pancreatic ductal carcinomas. Genomics 46: 284-286, 1997.; and
Donoviel, D. B.; Bernstein, A.: SEL-1L maps to human chromosome 14, near the insulin-dependent diabetes mellitus locus 11. Genomics 56: 232-233, 1999.

Further studies establishing the function and utilities of SEL1L are found in John Hopkins OMIM database record ID 602329, and in sited publications numbered 1400-1403 listed in the bibliography section hereinbelow, which are also hereby incorporated by reference. DKFZP56401863 (Accession XM_{—}043922) is another VGAM99 host target gene. DKFZP56401863 BINDING SITE is HOST TARGET binding site found in the 3′ untranslated region of mRNA encoded by DKFZP56401863, corresponding to a HOST TARGET binding site such as BINDING SITE I, BINDING SITE II or BINDING SITE III. Table 2 illustrates the complementarity of the nucleotide sequences of DKFZP56401863 BINDING SITE, designated SEQ ID:2826, to the nucleotide sequence of VGAM99 RNA, herein designated VGAM RNA, also designated SEQ ID:434.

Another function of VGAM99 is therefore inhibition of DKFZP56401863 (Accession XM_{—}043922). Accordingly, utilities of VGAM99 include diagnosis, prevention and treatment of diseases and clinical conditions associated with DKFZP56401863. LOC129446 (Accession XM_{—}072203) is another VGAM99 host target gene. LOC129446 BINDING SITE is HOST TARGET binding site found in the 3′ untranslated region of mRNA encoded by LOC129446, corresponding to a HOST TARGET binding site such as BINDING SITE I, BINDING SITE II or BINDING SITE III. Table 2 illustrates the complementarity of the nucleotide sequences of LOC129446 BINDING SITE, designated SEQ ID:3042, to the nucleotide sequence of VGAM99 RNA, herein designated VGAM RNA, also designated SEQ ID:434.

Another function of VGAM99 is therefore inhibition of LOC129446 (Accession XM_{—}072203). Accordingly, utilities of VGAM99 include diagnosis, prevention and treatment of diseases and clinical conditions associated with LOC129446. FIG. 1 further provides a conceptual description of a novel bioinformatically detected viral gene of the present invention, referred to here as Viral Genomic Address Messenger 100 (VGAM100) viral gene, which modulates expression of respective host target genes thereof, the function and utility of which host target genes is known in the art.

VGAM100 is a novel bioinformatically detected regulatory, non protein coding, viral micro RNA (miRNA) gene. The method by which VGAM100 was detected is described hereinabove with reference to FIGS. 1-8.

VGAM100 gene, herein designated VGAM GENE, is a viral gene contained in the genome of Vaccinia Virus. VGAM100 host target gene, herein designated VGAM HOST TARGET GENE, is a human gene contained in the human genome.

VGAM100 gene encodes a VGAM100 precursor RNA, herein designated VGAM PRECURSOR RNA. Similar to other miRNA genes, and unlike most ordinary genes, VGAM100 precursor RNA does not encode a protein. A nucleotide sequence identical or highly similar to the nucleotide sequence of VGAM100 precursor RNA is designated SEQ ID:86, and is provided hereinbelow with reference to the sequence listing part. Nucleotide sequence SEQ ID:86 is located at position 36878 relative to the genome of Vaccinia Virus.

VGAM100 precursor RNA folds onto itself, forming VGAM100 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM100 folded precursor RNA into VGAM100 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM100 RNA is designated SEQ ID:435, and is provided hereinbelow with reference to the sequence listing part.

VGAM100 host target gene, herein designated VGAM HOST TARGET GENE, encodes a corresponding messenger RNA, VGAM100 host target RNA, herein designated VGAM HOST TARGET RNA. VGAM100 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM100 RNA, herein designated VGAM RNA, binds complementarily to one or more host target binding sites located in untranslated regions of VGAM100 host target RNA, herein designated VGAM HOST TARGET RNA. This complementary binding is due to the fact that the nucleotide sequence of VGAM100 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM100 RNA, herein designated VGAM RNA, may have a different number of host target binding sites in untranslated regions of a VGAM100 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM100 RNA, herein designated VGAM RNA, to host target binding sites on VGAM100 host target RNA, herein designated VGAM HOST TARGET RNA, such as BINDING SITE I, BINDING SITE II and BINDING SITE III, inhibits translation of VGAM100 host target RNA into VGAM100 host target protein, herein designated VGAM HOST TARGET PROTEIN. VGAM host target protein is therefore outlined by a broken line.

It is appreciated that VGAM100 host target gene, herein designated VGAM HOST TARGET GENE, in fact represents a plurality of VGAM100 host target genes. The mRNA of each one of this plurality of VGAM100 host target genes comprises one or more host target binding sites, each having a nucleotide sequence which is at least partly complementary to VGAM100 RNA, herein designated VGAM RNA, and which when bound by VGAM100 RNA causes inhibition of translation of respective one or more VGAM100 host target proteins.

It is further appreciated by one skilled in the art that the mode of translational inhibition illustrated by FIG. 1 with specific reference to translational inhibition exerted by VGAM100 gene, herein designated VGAM GENE, on one or more VGAM100 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM100 is inhibition of expression of host target genes, as part of a novel viral mechanism of attacking a host. Accordingly, utilities of VGAM100 include diagnosis, prevention and treatment of viral infection by Vaccinia Virus. Specific functions, and accordingly utilities, of VGAM100 correlate with, and may be deduced from, the identity of the host target genes which VGAM100 binds and inhibits, and the function of these host target genes, as elaborated hereinbelow.

Nucleotide sequences of the VGAM100 precursor RNA, herein designated VGAM PRECURSOR RNA, and of the ‘diced’ VGAM100 RNA, herein designated VGAM RNA, and a schematic representation of the secondary folding of VGAM100 folded precursor RNA, herein designated VGAM FOLDED PRECURSOR RNA, of VGAM100 are further described hereinbelow with reference to Table 1.

Nucleotide sequences of host target binding sites, such as BINDING SITE-I, BINDING SITE-II and BINDING SITE-III of FIG. 1, found on VGAM100 host target RNA, and schematic representation of the complementarity of each of these host target binding sites to VGAM100 RNA, herein designated VGAM RNA, are described hereinbelow with reference to Table 2.

As mentioned hereinabove with reference to FIG. 1, a function of VGAM100 gene, herein designated VGAM is inhibition of expression of VGAM100 target genes. It is appreciated that specific functions, and accordingly utilities, of VGAM100 correlate with, and may be deduced from, the identity of the target genes which VGAM100 binds and inhibits, and the function of these target genes, as elaborated hereinbelow.

Cytochrome P450, Sub family XIX (aromatization of androgens) (CYP19, Accession NM_{—}000103) is a VGAM100 host target gene. CYP19 BINDING SITE1 and CYP19 BINDING SITE2 are HOST TARGET binding sites found in untranslated regions of mRNA encoded by CYP19, corresponding to HOST TARGET binding sites such as BINDING SITE I, BINDING SITE II or BINDING SITE III. Table 2 illustrates the complementarity of the nucleotide sequences of CYP19 BINDING SITE1 and CYP19 BINDING SITE2, designated SEQ ID:705 and SEQ ID:2185 respectively, to the nucleotide sequence of VGAM100 RNA, herein designated VGAM RNA, also designated SEQ ID:435.

A function of VGAM100 is therefore inhibition of Cytochrome P450, Sub family XIX (aromatization of androgens) (CYP19, Accession NM_{—}000103), a gene which catalyzes the last steps of estrogen biosynthesis. Accordingly, utilities of VGAM100 include diagnosis, prevention and treatment of diseases and clinical conditions associated with CYP19. The function of CYP19 has been established by previous studies. The distinct gender-specific patterns of fat distribution in men and women (android and gynoid) suggest a role for sex steroids. It has been suggested that estrogens can promote preadipocyte cell proliferation and/or differentiation. The enzyme CYP19 is responsible for the conversion of androgen precursor steroids to estrogens and may, therefore, have a role in regulating adipose tissue mass and its distribution. McTernan et al. (2002) investigated the glucocorticoid regulation of aromatase expression in adipose tissue, specifically to define any site- and gender-specific differences. Abdominal subcutaneous and omental adipose tissue was obtained from male and female patients undergoing elective surgery. Cortisol-induced aromatase activity in omental adipocytes from postmenopausal females was higher than that in premenopausal females (P less than 0.001). Insulin had no independent effect on aromatase expression, but coincubation of preadipocytes with cortisol and insulin eliminated both gender- and site-specific differences. The authors concluded that in women, but not men, cortisol increases aromatase activity at subcutaneous sites, and this may facilitate predilection for subcutaneous adiposity in females. They suggested that the observed site-, gender-, and menopausal-specific differences in the glucocorticoid regulation of this enzyme may contribute to the gender- and menopausal-specific patterns of fat distribution. Hemsell et al. (1977) reported a case of gynecomastia apparently due to excessive peripheral conversion of androgen to estrogen as a result of 50-times-normal aromatase activity. The patient was an adopted boy, aged 11 years 7 months. Effects of excessive estrogen became evident at age 8, the time when plasma androstenedione begins to increase. Extraglandular aromatization, as well as sulfurylation, is extensively involved in C19-steroid metabolism in the fetus, but the activity of the enzymes falls rapidly after birth. In the patient of Hemsell et al. (1977), the fetal situation appeared to persist. Berkovitz et al. (1985) investigated a black family in which marked gynecomastia with normal male genitalia occurred in 5 men in 3 sibships of 2 generations connected through females. In each, gynecomastia and male sexual differentiation began at an early age (10 to 11 years). The ratio of the concentration of plasma estradiol-17 beta to that of plasma testosterone was elevated in each. In 3 affected sibs, the transfer constant of conversion of androstenedione to estrone (i.e., the fraction of plasma androstenedione that was converted to estrone as measured in the urine) was 10 times the normal. Despite elevated extraglandular aromatase activity, the hypothalamic-pituitary axis responded normally to provocative stimuli. None of the 5 males had children, but 4 were still in their teens; the fifth was 29 years of age. The pattern of inheritance of familial gynecomastia with increased aromatase activity is consistent with either X-linked recessive or autosomal dominant, male-limited inheritance. Mapping of the aromatase locus to an autosome makes the latter possibility highly likely. Autosomal dominant inheritance appeared likely in a family, reported by Leiberman and Zachmann (1992), in which increased steroid aromatization seemed to be responsible for ‘familial adrenal feminization.’ The father and 2 male and 2 female sibs had gynecomastia, early growth, and short final stature. The 8-year-old propositus had advanced bone age, facial acne, gynecomastia, pubic hair, and prepubertal testicular volume. ACTH-dependent adrenal feminization was confirmed by a transient reduction of breast tissue following dexamethasone or cypropterone acetate treatment. Testolactone, which is an inhibitor of peripheral aromatase activity in vivo, temporarily reduced the breast tissue. This was the first example of male-to-male and male-to-female transmission reported. Animal model experiments lend further support to the function of CYP19. Aromatase knockout (ArKO) mice, lacking a functional Cyp19 gene, cannot synthesize endogenous estrogens. Jones et al. (2000) examined the adipose deposits of male and female ArKO mice, observing that these animal progressively accumulated significantly more intraabdominal adipose tissue than their wildtype littermates, reflected in increased adipocyte volume at gonadal and infrarenal sites. This increased adiposity was not due to hyperphagia or reduced resting energy expenditure, but was associated with reduced spontaneous physical activity levels, reduced glucose oxidation, and a decrease in lean body mass. A striking accumulation of lipid droplets was observed in the livers of ArKO animals. The findings demonstrated an important role for estrogen in the maintenance of lipid homeostasis in both males and females. Along the same lines, Heine et al. (2000) studied male and female Esr1 knockout mice and found that signaling by this receptor is critical in female and male white adipose tissue. Obesity in the males involved a mechanism of reduced energy expenditure rather than increased energy intake.

It is appreciated that the abovementioned animal model for CYP19 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Deladoey, J.; Fluck, C.; Bex, M.; Yoshimura, N.; Harada, N.; Mullis, P. E.: Aromatase deficiency caused by a novel P450(arom) gene mutation: impact of absent estrogen production on serum gonadotropin concentration in a boy. J. Clin. Endocr. Metab. 84: 4050-4054, 1999.; and
Yang, S.; Fang, Z.; Suzuki, T.; Sasano, H.; Zhou, J.; Gurates, B.; Tamura, M.; Ferrer, K.; Bulun, S.: Regulation of aromatase P450 expression in endometriotic and endometrial stromal.

Further studies establishing the function and utilities of CYP19 are found in John Hopkins OMIM database record ID 107910, and in sited publications numbered 1052, 1076-1091, 497, 1092-1093, 201, 1094-1103, 5, 3020-3021, 69 and 3022-3026 listed in the bibliography section hereinbelow, which are also hereby incorporated by reference. Macrophage Scavenger Receptor 1 (MSR1, Accession NM_{—}138715) is another VGAM100 host target gene. MSR1 BINDING SITE1 and MSR1 BINDING SITE2 are HOST TARGET binding sites found in untranslated regions of mRNA encoded by MSR1, corresponding to HOST TARGET binding sites such as BINDING SITE I, BINDING SITE II or BINDING SITE III. Table 2 illustrates the complementarity of the nucleotide sequences of MSR1 BINDING SITE1 and MSR1 BINDING SITE2, designated SEQ ID:2453 and SEQ ID:2454 respectively, to the nucleotide sequence of VGAM100 RNA, herein designated VGAM RNA, also designated SEQ ID:435.

Another function of VGAM100 is therefore inhibition of Macrophage Scavenger Receptor 1 (MSR1, Accession NM_{—}138715), a gene which plays a role in endocytosis of macromolecules. Accordingly, utilities of VGAM100 include diagnosis, prevention and treatment of diseases and clinical conditions associated with MSR1. The function of MSR1 and its association with various diseases and clinical conditions, has been established by previous studies, as described hereinabove with reference to VGAM49. Glutaminyl-peptide Cyclotransferase (glutaminyl cyclase) (QPCT, Accession NM_{—}012413) is another VGAM100 host target gene. QPCT BINDING SITE is HOST TARGET binding site found in the 3′ untranslated region of mRNA encoded by QPCT, corresponding to a HOST TARGET binding site such as BINDING SITE I, BINDING SITE II or BINDING SITE III. Table 2 illustrates the complementarity of the nucleotide sequences of QPCT BINDING SITE, designated SEQ ID: 1428, to the nucleotide sequence of VGAM100 RNA, herein designated VGAM RNA, also designated SEQ ID:435.

Another function of VGAM100 is therefore inhibition of Glutaminyl-peptide Cyclotransferase (glutaminyl cyclase) (QPCT, Accession NM_{—}012413). Accordingly, utilities of VGAM100 include diagnosis, prevention and treatment of diseases and clinical conditions associated with QPCT. Twist Homolog (acrocephalosyndactyly 3; Saethre-Chotzen syndrome) (Drosophila) (TWIST, Accession NM_{—}000474) is another VGAM100 host target gene. TWIST BINDING SITE is HOST TARGET binding site found in the 3′ untranslated region of mRNA encoded by TWIST, corresponding to a HOST TARGET binding site such as BINDING SITE I, BINDING SITE II or BINDING SITE III. Table 2 illustrates the complementarity of the nucleotide sequences of TWIST BINDING SITE, designated SEQ ID:750, to the nucleotide sequence of VGAM100 RNA, herein designated VGAM RNA, also designated SEQ ID:435.

Another function of VGAM100 is therefore inhibition of Twist Homolog (acrocephalosyndactyly 3; Saethre-Chotzen syndrome) (Drosophila) (TWIST, Accession NM_{—}000474). Accordingly, utilities of VGAM100 include diagnosis, prevention and treatment of diseases and clinical conditions associated with TWIST. ATPase, Class II, Type 9A (ATP9A, Accession XM_{—}030577) is another VGAM100 host target gene. ATP9A BINDING SITE is HOST TARGET binding site found in the 3′ untranslated region of mRNA encoded by ATP9A, corresponding to a HOST TARGET binding site such as BINDING SITE I, BINDING SITE II or BINDING SITE III. Table 2 illustrates the complementarity of the nucleotide sequences of ATP9A BINDING SITE, designated SEQ ID:2617, to the nucleotide sequence of VGAM100 RNA, herein designated VGAM RNA, also designated SEQ ID:435.

Another function of VGAM100 is therefore inhibition of ATPase, Class II, Type 9A (ATP9A, Accession XM_{—}030577). Accordingly, utilities of VGAM100 include diagnosis, prevention and treatment of diseases and clinical conditions associated with ATP9A. Calneuron 1 (CALN1, Accession NM_{—}031468) is another VGAM100 host target gene. CALN1 BINDING SITE is HOST TARGET binding site found in the 3′ untranslated region of mRNA encoded by CALN1, corresponding to a HOST TARGET binding site such as BINDING SITE I, BINDING SITE II or BINDING SITE III. Table 2 illustrates the complementarity of the nucleotide sequences of CALN1 BINDING SITE, designated SEQ ID:2207, to the nucleotide sequence of VGAM100 RNA, herein designated VGAM RNA, also designated SEQ ID:435.

Another function of VGAM100 is therefore inhibition of Calneuron 1 (CALN1, Accession NM_{—}031468). Accordingly, utilities of VGAM100 include diagnosis, prevention and treatment of diseases and clinical conditions associated with CALN1. FLJ23277 (Accession NM_{—}032236) is another VGAM100 host target gene. FLJ23277 BINDING SITE is HOST TARGET binding site found in the 3′ untranslated region of mRNA encoded by FLJ23277, corresponding to a HOST TARGET binding site such as BINDING SITE I, BINDING SITE II or BINDING SITE III. Table 2 illustrates the complementarity of the nucleotide sequences of FLJ23277 BINDING SITE, designated SEQ ID:2238, to the nucleotide sequence of VGAM100 RNA, herein designated VGAM RNA, also designated SEQ ID:435.

Another function of VGAM100 is therefore inhibition of FLJ23277 (Accession NM_{—}032236). Accordingly, utilities of VGAM100 include diagnosis, prevention and treatment of diseases and clinical conditions associated with FLJ23277. KIAA0992 (Accession NM_{—}016081) is another VGAM100 host target gene. KIAA0992 BINDING SITE is HOST TARGET binding site found in the 3′ untranslated region of mRNA encoded by KIAA0992, corresponding to a HOST TARGET binding site such as BINDING SITE I, BINDING SITE II or BINDING SITE III. Table 2 illustrates the complementarity of the nucleotide sequences of KIAA0992 BINDING SITE, designated SEQ ID: 1661, to the nucleotide sequence of VGAM100 RNA, herein designated VGAM RNA, also designated SEQ ID:435.

Another function of VGAM100 is therefore inhibition of KIAA0992 (Accession NM_{—}016081). Accordingly, utilities of VGAM100 include diagnosis, prevention and treatment of diseases and clinical conditions associated with KIAA0992. KIAA1045 (Accession XM_{—}048592) is another VGAM100 host target gene. KIAA1045 BINDING SITE is HOST TARGET binding site found in the 3′ untranslated region of mRNA encoded by KIAA1045, corresponding to a HOST TARGET binding site such as BINDING SITE I, BINDING SITE II or BINDING SITE III. Table 2 illustrates the complementarity of the nucleotide sequences of KIAA1045 BINDING SITE, designated SEQ ID:2906, to the nucleotide sequence of VGAM100 RNA, herein designated VGAM RNA, also designated SEQ ID:435.

Another function of VGAM100 is therefore inhibition of KIAA1045 (Accession XM_{—}048592). Accordingly, utilities of VGAM100 include diagnosis, prevention and treatment of diseases and clinical conditions associated with KIAA1045. KIAA1056 (Accession NM_{—}014894) is another VGAM100 host target gene. KIAA1056 BINDING SITE is HOST TARGET binding site found in the 3′ untranslated region of mRNA encoded by KIAA1056, corresponding to a HOST TARGET binding site such as BINDING SITE I, BINDING SITE II or BINDING SITE III. Table 2 illustrates the complementarity of the nucleotide sequences of KIAA1056 BINDING SITE, designated SEQ ID:1575, to the nucleotide sequence of VGAM100 RNA, herein designated VGAM RNA, also designated SEQ ID:435.

Another function of VGAM100 is therefore inhibition of KIAA1056 (Accession NM_{—}014894). Accordingly, utilities of VGAM100 include diagnosis, prevention and treatment of diseases and clinical conditions associated with KIAA1056. KIAA1323 (Accession XM_{—}032146) is another VGAM100 host target gene. KIAA1323 BINDING SITE is HOST TARGET binding site found in the 3′ untranslated region of mRNA encoded by KIAA1323, corresponding to a HOST TARGET binding site such as BINDING SITE I, BINDING SITE II or BINDING SITE III. Table 2 illustrates the complementarity of the nucleotide sequences of KIAA1323 BINDING SITE, designated SEQ ID:2646, to the nucleotide sequence of VGAM100 RNA, herein designated VGAM RNA, also designated SEQ ID:435.

Another function of VGAM100 is therefore inhibition of KIAA1323 (Accession XM_{—}032146). Accordingly, utilities of VGAM100 include diagnosis, prevention and treatment of diseases and clinical conditions associated with KIAA1323. Leiomodin 1 (smooth muscle) (LMOD1, Accession NM_{—}012134) is another VGAM100 host target gene. LMOD1 BINDING SITE is HOST TARGET binding site found in the 3′ untranslated region of mRNA encoded by LMOD1, corresponding to a HOST TARGET binding site such as BINDING SITE I, BINDING SITE II or BINDING SITE III. Table 2 illustrates the complementarity of the nucleotide sequences of LMOD1 BINDING SITE, designated SEQ ID: 1409, to the nucleotide sequence of VGAM100 RNA, herein designated VGAM RNA, also designated SEQ ID:435.

Another function of VGAM100 is therefore inhibition of Leiomodin 1 (smooth muscle) (LMOD1, Accession NM_{—}012134). Accordingly, utilities of VGAM100 include diagnosis, prevention and treatment of diseases and clinical conditions associated with LMOD1. Ring Finger Protein 32 (RNF32, Accession NM_{—}030936) is another VGAM100 host target gene. RNF32 BINDING SITE is HOST TARGET binding site found in the 3′ untranslated region of mRNA encoded by RNF32, corresponding to a HOST TARGET binding site such as BINDING SITE I, BINDING SITE II or BINDING SITE III. Table 2 illustrates the complementarity of the nucleotide sequences of RNF32 BINDING SITE, designated SEQ ID:2172, to the nucleotide sequence of VGAM100 RNA, herein designated VGAM RNA, also designated SEQ ID:435.

Another function of VGAM100 is therefore inhibition of Ring Finger Protein 32 (RNF32, Accession NM_{—}030936). Accordingly, utilities of VGAM100 include diagnosis, prevention and treatment of diseases and clinical conditions associated with RNF32. SS-56 (Accession XM_{—}006063) is another VGAM100 host target gene. SS-56 BINDING SITE is HOST TARGET binding site found in the 3′ untranslated region of mRNA encoded by SS-56, corresponding to a HOST TARGET binding site such as BINDING SITE I, BINDING SITE II or BINDING SITE III. Table 2 illustrates the complementarity of the nucleotide sequences of SS-56 BINDING SITE, designated SEQ ID:2535, to the nucleotide sequence of VGAM100 RNA, herein designated VGAM RNA, also designated SEQ ID:435.

Another function of VGAM100 is therefore inhibition of SS-56 (Accession XM_{—}006063). Accordingly, utilities of VGAM100 include diagnosis, prevention and treatment of diseases and clinical conditions associated with SS-56. LOC122792 (Accession NM_{—}145251) is another VGAM100 host target gene. LOC122792 BINDING SITE is HOST TARGET binding site found in the 3′ untranslated region of mRNA encoded by LOC122792, corresponding to a HOST TARGET binding site such as BINDING SITE I, BINDING SITE II or BINDING SITE III. Table 2 illustrates the complementarity of the nucleotide sequences of LOC122792 BINDING SITE, designated SEQ ID:2518, to the nucleotide sequence of VGAM100 RNA, herein designated VGAM RNA, also designated SEQ ID:435.

Another function of VGAM100 is therefore inhibition of LOC122792 (Accession NM_{—}145251). Accordingly, utilities of VGAM100 include diagnosis, prevention and treatment of diseases and clinical conditions associated with LOC122792. LOC146512 (Accession XM_{—}085490) is another VGAM100 host target gene. LOC146512 BINDING SITE is HOST TARGET binding site found in the 3′ untranslated region of mRNA encoded by LOC146512, corresponding to a HOST TARGET binding site such as BINDING SITE I, BINDING SITE II or BINDING SITE III. Table 2 illustrates the complementarity of the nucleotide sequences of LOC146512 BINDING SITE, designated SEQ ID:3094, to the nucleotide sequence of VGAM100 RNA, herein designated VGAM RNA, also designated SEQ ID:435.

Another function of VGAM100 is therefore inhibition of LOC146512 (Accession XM_{—}085490). Accordingly, utilities of VGAM100 include diagnosis, prevention and treatment of diseases and clinical conditions associated with LOC146512. LOC201633 (Accession XM_{—}117317) is another VGAM100 host target gene. LOC201633 BINDING SITE is HOST TARGET binding site found in the 3′ untranslated region of mRNA encoded by LOC201633, corresponding to a HOST TARGET binding site such as BINDING SITE I, BINDING SITE II or BINDING SITE III. Table 2 illustrates the complementarity of the nucleotide sequences of LOC201633 BINDING SITE, designated SEQ ID:3483, to the nucleotide sequence of VGAM100 RNA, herein designated VGAM RNA, also designated SEQ ID:435.

Another function of VGAM100 is therefore inhibition of LOC201633 (Accession XM_{—}117317). Accordingly, utilities of VGAM100 include diagnosis, prevention and treatment of diseases and clinical conditions associated with LOC201633. LOC202126 (Accession XM_{—}117362) is another VGAM100 host target gene. LOC202126 BINDING SITE is HOST TARGET binding site found in the 3′ untranslated region of mRNA encoded by LOC202126, corresponding to a HOST TARGET binding site such as BINDING SITE I, BINDING SITE II or BINDING SITE III. Table 2 illustrates the complementarity of the nucleotide sequences of LOC202126 BINDING SITE, designated SEQ ID:3486, to the nucleotide sequence of VGAM100 RNA, herein designated VGAM RNA, also designated SEQ ID:435.

Another function of VGAM100 is therefore inhibition of LOC202126 (Accession XM_{—}117362). Accordingly, utilities of VGAM100 include diagnosis, prevention and treatment of diseases and clinical conditions associated with LOC202126. LOC221755 (Accession XM_{—}166465) is another VGAM100 host target gene. LOC221755 BINDING SITE is HOST TARGET binding site found in the 3′ untranslated region of mRNA encoded by LOC221755, corresponding to a HOST TARGET binding site such as BINDING SITE I, BINDING SITE II or BINDING SITE III. Table 2 illustrates the complementarity of the nucleotide sequences of LOC221755 BINDING SITE, designated SEQ ID:3563, to the nucleotide sequence of VGAM100 RNA, herein designated VGAM RNA, also designated SEQ ID:435.

Another function of VGAM100 is therefore inhibition of LOC221755 (Accession XM_{—}166465). Accordingly, utilities of VGAM100 include diagnosis, prevention and treatment of diseases and clinical conditions associated with LOC221755. LOC257334 (Accession XM_{—}173202) is another VGAM100 host target gene. LOC257334 BINDING SITE is HOST TARGET binding site found in the 3′ untranslated region of mRNA encoded by LOC257334, corresponding to a HOST TARGET binding site such as BINDING SITE I, BINDING SITE II or BINDING SITE III. Table 2 illustrates the complementarity of the nucleotide sequences of LOC257334 BINDING SITE, designated SEQ ID:3734, to the nucleotide sequence of VGAM100 RNA, herein designated VGAM RNA, also designated SEQ ID:435.

Another function of VGAM100 is therefore inhibition of LOC257334 (Accession XM_{—}173202). Accordingly, utilities of VGAM100 include diagnosis, prevention and treatment of diseases and clinical conditions associated with LOC257334. FIG. 1 further provides a conceptual description of a novel bioinformatically detected viral gene of the present invention, referred to here as Viral Genomic Address Messenger 101 (VGAM101) viral gene, which modulates expression of respective host target genes thereof, the function and utility of which host target genes is known in the art.

VGAM101 is a novel bioinformatically detected regulatory, non protein coding, viral micro RNA (miRNA) gene. The method by which VGAM101 was detected is described hereinabove with reference to FIGS. 1-8.

VGAM101 gene, herein designated VGAM GENE, is a viral gene contained in the genome of Vaccinia Virus. VGAM101 host target gene, herein designated VGAM HOST TARGET GENE, is a human gene contained in the human genome.

VGAM101 gene encodes a VGAM101 precursor RNA, herein designated VGAM PRECURSOR RNA. Similar to other miRNA genes, and unlike most ordinary genes, VGAM101 precursor RNA does not encode a protein. A nucleotide sequence identical or highly similar to the nucleotide sequence of VGAM101 precursor RNA is designated SEQ ID:87, and is provided hereinbelow with reference to the sequence listing part. Nucleotide sequence SEQ ID:87 is located at position 41548 relative to the genome of Vaccinia Virus.

VGAM101 precursor RNA folds onto itself, forming VGAM101 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM101 folded precursor RNA into VGAM101 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM101 RNA is designated SEQ ID:436, and is provided hereinbelow with reference to the sequence listing part.

VGAM101 host target gene, herein designated VGAM HOST TARGET GENE, encodes a corresponding messenger RNA, VGAM101 host target RNA, herein designated VGAM HOST TARGET RNA. VGAM101 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM101 RNA, herein designated VGAM RNA, binds complementarily to one or more host target binding sites located in untranslated regions of VGAM101 host target RNA, herein designated VGAM HOST TARGET RNA. This complementary binding is due to the fact that the nucleotide sequence of VGAM101 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM101 RNA, herein designated VGAM RNA, may have a different number of host target binding sites in untranslated regions of a VGAM101 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM101 RNA, herein designated VGAM RNA, to host target binding sites on VGAM101 host target RNA, herein designated VGAM HOST TARGET RNA, such as BINDING SITE I, BINDING SITE II and BINDING SITE III, inhibits translation of VGAM101 host target RNA into VGAM101 host target protein, herein designated VGAM HOST TARGET PROTEIN. VGAM host target protein is therefore outlined by a broken line.

It is appreciated that VGAM101 host target gene, herein designated VGAM HOST TARGET GENE, in fact represents a plurality of VGAM101 host target genes. The mRNA of each one of this plurality of VGAM101 host target genes comprises one or more host target binding sites, each having a nucleotide sequence which is at least partly complementary to VGAM101 RNA, herein designated VGAM RNA, and which when bound by VGAM101 RNA causes inhibition of translation of respective one or more VGAM101 host target proteins.

It is further appreciated by one skilled in the art that the mode of translational inhibition illustrated by FIG. 1 with specific reference to translational inhibition exerted by VGAM101 gene, herein designated VGAM GENE, on one or more VGAM101 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM101 is inhibition of expression of host target genes, as part of a novel viral mechanism of attacking a host. Accordingly, utilities of VGAM101 include diagnosis, prevention and treatment of viral infection by Vaccinia Virus. Specific functions, and accordingly utilities, of VGAM101 correlate with, and may be deduced from, the identity of the host target genes which VGAM101 binds and inhibits, and the function of these host target genes, as elaborated hereinbelow.

Nucleotide sequences of the VGAM101 precursor RNA, herein designated VGAM PRECURSOR RNA, and of the ‘diced’ VGAM101 RNA, herein designated VGAM RNA, and a schematic representation of the secondary folding of VGAM101 folded precursor RNA, herein designated VGAM FOLDED PRECURSOR RNA, of VGAM101 are further described hereinbelow with reference to Table 1.

Nucleotide sequences of host target binding sites, such as BINDING SITE-I, BINDING SITE-II and BINDING SITE-III of FIG. 1, found on VGAM101 host target RNA, and schematic representation of the complementarity of each of these host target binding sites to VGAM101 RNA, herein designated VGAM RNA, are described hereinbelow with reference to Table 2.

As mentioned hereinabove with reference to FIG. 1, a function of VGAM101 gene, herein designated VGAM is inhibition of expression of VGAM101 target genes. It is appreciated that specific functions, and accordingly utilities, of VGAM101 correlate with, and may be deduced from, the identity of the target genes which VGAM101 binds and inhibits, and the function of these target genes, as elaborated hereinbelow.

Angiopoietin 1 (ANGPT1, Accession NM_{—}001146) is a VGAM101 host target gene. ANGPT1 BINDING SITE is HOST TARGET binding site found in the 3′ untranslated region of mRNA encoded by ANGPT1, corresponding to a HOST TARGET binding site such as BINDING SITE I, BINDING SITE II or BINDING SITE III. Table 2 illustrates the complementarity of the nucleotide sequences of ANGPT1 BINDING SITE, designated SEQ ID:804, to the nucleotide sequence of VGAM101 RNA, herein designated VGAM RNA, also designated SEQ ID:436.

A function of VGAM101 is therefore inhibition of Angiopoietin 1 (ANGPT1, Accession NM_{—}001146), a gene which binds and activates tie2 receptor by inducing its tyrosine phosphorylation. Accordingly, utilities of VGAM101 include diagnosis, prevention and treatment of diseases and clinical conditions associated with ANGPT1. The function of ANGPT1 has been established by previous studies. By FISH and radiation hybrid analysis, Cheung et al. (1998) mapped the ANGPT1 gene to 8q22.3-q23. By FISH, Valenzuela et al. (1999) mapped the ANGPT1 gene to 8q22 in a region that shows homology of synteny to mouse chromosome 15, where they mapped the mouse Angpt1 gene. However, by indirect in situ PCR and FISH, Marziliano et al. (1999) mapped the Angpt1 gene in the mouse to chromosome 9E2. To explore the possibility that VEGF and angiopoietins collaborate during tumor angiogenesis, Holash et al. (1999) analyzed several different murine and human tumor models. The apparent association of tumor vessel regression, apoptosis, and disruption of endothelial cell interactions with support cells in rat C6 gliomas raised the possibility that blockade of the stabilizing action of Ang1 might be contributing to tumor vessel regression. Consistent with this possibility, Holash et al. (1999) noted that angiopoietin-1 was antiapoptotic for cultured endothelial cells and expression of its antagonist angiopoietin-2 was induced in the endothelium of co-opted tumor vessels before their regression. Diffuse angiopoietin-1 expression in human tumors resembled that seen in the rat model. Holash et al. (1999) suggested that a subset of tumors rapidly co-opts existing host vessels to form an initially well vascularized tumor mass. Perhaps as part of a host defense mechanism there is widespread regression of these initially co-opted vessels, leading to a secondarily avascular tumor and a massive tumor cell loss. However, the remaining tumor is ultimately rescued by robust angiogenesis at the tumor margin Animal model experiments lend further support to the function of ANGPT1. Suri et al. (1996) showed that mice engineered to lack angiopoietin-1 display angiogenic defects reminiscent of those previously seen in mice lacking Tie2, demonstrating that angiopoietin-1 is a primary physiologic ligand for Tie2 and that it has critical in vivo angiogenic actions that are distinct from vascular endothelial growth factor (VEGF; 192240) and that are not reflected in the classic in vitro assays used to characterize VEGF. They concluded that angiopoietin-1 appears to play a crucial role in mediating reciprocal interactions between the endothelium and surrounding matrix and mesenchyme.

It is appreciated that the abovementioned animal model for ANGPT1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Holash, J.; Maisonpierre, P. C.; Compton, D.; Boland, P.; Alexander, C. R.; Zagzag, D.; Yancopoulos, G. D.; Wiegand, S. J.: Vessel cooption, regression, and growth in tumors mediated by angiopoietins and VEGF. Science 284: 1994-1998, 1999.; and
Suri, C.; Jones, P. F.; Patan, S.; Bartunkova, S.; Maisonpierre, P. C.; Davis, S.; Sato, T. N.; Yancopoulos, G. D.: Requisite role of angiopoietin-1, a ligand for the TIE2 receptor, du.

Further studies establishing the function and utilities of ANGPT1 are found in John Hopkins OMIM database record ID 601667, and in sited publications numbered 2124-2126, 2373, 2127-2130, 237 and 2328 listed in the bibliography section hereinbelow, which are also hereby incorporated by reference. G Protein-coupled Receptor 61 (GPR61, Accession XM_{—}086232) is another VGAM101 host target gene. GPR61 BINDING SITE is HOST TARGET binding site found in the 5′ untranslated region of mRNA encoded by GPR61, corresponding to a HOST TARGET binding site such as BINDING SITE I, BINDING SITE II or BINDING SITE III. Table 2 illustrates the complementarity of the nucleotide sequences of GPR61 BINDING SITE, designated SEQ ID:3123, to the nucleotide sequence of VGAM101 RNA, herein designated VGAM RNA, also designated SEQ ID:436.

Another function of VGAM101 is therefore inhibition of G Protein-coupled Receptor 61 (GPR61, Accession XM_{—}086232), a gene which transduces extracellular signals through heterotrimeric G proteins. Accordingly, utilities of VGAM101 include diagnosis, prevention and treatment of diseases and clinical conditions associated with GPR61. The function of GPR61 has been established by previous studies. By PCR of genomic DNA, Lee et al. (2001) obtained a partial GPR61 clone using primers designed from the sequence of a rabbit G protein-coupled receptor (GPCR). They used the PCR products to screen a human brain cDNA library and found that GPR61 encodes a 417-amino acid protein that shares over 80% identity with the rabbit homolog and 44% similarity with GPR62 (OMIM Ref. No. 606917) in the transmembrane region. By Northern blot analysis of human brain areas, Lee et al. (2001) detected a 4.3-kb transcript in caudate, putamen, and thalamus, but not in hypothalamus, hippocampus, pons, frontal cortex, or cerebellum. No expression was found in liver or kidney. Lee et al. (2001) also cloned rat GPR61 and, by in situ hybridization, found widespread expression in rat brain. Cikos et al. (2001) independently cloned GPR61, which they called BALGR. A partial sequence was obtained by RT-PCR of human brain mRNA using degenerate oligonucleotides corresponding to a transmembrane sequence of GPR30 (OMIM Ref. No. 601805). They cloned the full-length cDNA from a hypothalamus cDNA library. They found that the deduced protein contains 451 amino acids and has a calculated molecular mass of about 49 kD. GPR61 contains 7 putative transmembrane domains, a potential N-glycosylation site, cysteine residues that may form a disulfide bridge, and several putative protein kinase sites. It also contains a conserved glu-arg-tyr sequence shared with other GPCRs. The highest sequence similarity (OMIM Ref. No. 28-31%) was found with biogenic amine GPCRs, i.e., serotonin, histamine, adrenergic, and dopamine GPCRs. By semiquantitative PCR of multiple human tissues, Cikos et al. (2001) found highest expression in brain and testes and low but detectable expression in all other tissues examined. Their results of Northern blot analysis of human brain regions differed from that reported by Lee et al. (2001). Strong expression of a 4.8-kb transcript was found in cerebral cortex, occipital pole, frontal lobe, temporal lobe, amygdala, and hippocampus, and lower expression in putamen and caudate nucleus. No message was detected in cerebellum, medulla oblongata, spinal cord, corpus callosum, substantia nigra, subthalamic nucleus, or thalamus.

Full details of the abovementioned studies are described in the following publications, the disclosure of which are hereby incorporated by reference:
Cikos, S.; Gregor, P.; Koppel, J.: Cloning of a novel biogenic amine receptor-like G protein-coupled receptor expressed in human brain. Biochim. Biophys. Acta 1521: 66-72, 2001.; and
Lee, D. K.; George, S. R.; Cheng, R.; Nguyen, T.; Liu, Y.; Brown, M.; Lynch, K. R.; O'Dowd, B. F.: Identification of four novel human G protein-coupled receptors expressed in the brai.

Further studies establishing the function and utilities of GPR61 are found in John Hopkins OMIM database record ID 606916, and in sited publications numbered 1254 listed in the bibliography section hereinbelow, which are also hereby incorporated by reference. Protein Phosphatase 1, Regulatory (inhibitor) Subunit 12B (PPP1R12B, Accession NM_{—}032105) is another VGAM101 host target gene. PPP1R12B BINDING SITE is HOST TARGET binding site found in the 3′ untranslated region of mRNA encoded by PPP1R12B, corresponding to a HOST TARGET binding site such as BINDING SITE I, BINDING SITE II or BINDING SITE III. Table 2 illustrates the complementarity of the nucleotide sequences of PPP1R12B BINDING SITE, designated SEQ ID:2228, to the nucleotide sequence of VGAM101 RNA, herein designated VGAM RNA, also designated SEQ ID:436.

Another function of VGAM101 is therefore inhibition of Protein Phosphatase 1, Regulatory (inhibitor) Subunit 12B (PPP1R12B, Accession NM_{—}032105). Accordingly, utilities of VGAM101 include diagnosis, prevention and treatment of diseases and clinical conditions associated with PPP1R12B. Tripartite Motif-containing 9 (TRIM9, Accession NM_{—}015163) is another VGAM101 host target gene. TRIM9 BINDING SITE is HOST TARGET binding site found in the 3′ untranslated region of mRNA encoded by TRIM9, corresponding to a HOST TARGET binding site such as BINDING SITE I, BINDING SITE II or BINDING SITE III. Table 2 illustrates the complementarity of the nucleotide sequences of TRIM9 BINDING SITE, designated SEQ ID:1610, to the nucleotide sequence of VGAM101 RNA, herein designated VGAM RNA, also designated SEQ ID:436.

Another function of VGAM101 is therefore inhibition of Tripartite Motif-containing 9 (TRIM9, Accession NM_{—}015163), a gene which may function as a positive regulator for mannosylphosphate transferase and is required to mediate mannosylphosphate transfer in both the core and outer chain portions of n-linked. oligosaccharides. Accordingly, utilities of VGAM101 include diagnosis, prevention and treatment of diseases and clinical conditions associated with TRIM9. The function of TRIM9 has been established by previous studies. TRIM proteins are composed of 3 zinc-binding domains, a RING, a B-box type I, and a B-box type 2, followed by a coiled-coil region. They are involved in development and cell growth. By EST database searching for B-box-containing proteins, Reymond et al. (2001) identified 37 TRIM members, including 3 isoforms of TRIM9. Northern blot analysis revealed high expression of a 4.4-kb TRIM9 transcript in brain. Fluorescence microscopy demonstrated expression of TRIM9 in cytoplasmic speckles. Interaction mating analysis indicated that TRIM9 can form a homodimer.

Full details of the abovementioned studies are described in the following publications, the disclosure of which are hereby incorporated by reference:
Li, Y.; Chin, L.-S.; Weigel, C.; Li, L.: Spring, a novel RING finger protein that regulates synaptic vesicle exocytosis. J. Biol. Chem. 276: 40824-40833, 2001.; and
Reymond, A.; Meroni, G.; Fantozzi, A.; Merla, G.; Cairo, S.; Luzi, L.; Riganelli, D.; Zanaria, E.; Messali, S.; Cainarca, S.; Guffanti, A.; Minucci, S.; Pelicci, P. G.; Ballabio, A.:.

Further studies establishing the function and utilities of TRIM9 are found in John Hopkins OMIM database record ID 606555, and in sited publications numbered 1009-1010 listed in the bibliography section hereinbelow, which are also hereby incorporated by reference. Tyrosine 3-monooxygenase/tryptophan 5-monooxygenase Activation Protein, Epsilon Polypeptide (YWHAE, Accession NM_{—}006761) is another VGAM101 host target gene. YWHAE BINDING SITE is HOST TARGET binding site found in the 3′ untranslated region of mRNA encoded by YWHAE, corresponding to a HOST TARGET binding site such as BINDING SITE I, BINDING SITE II or BINDING SITE III. Table 2 illustrates the complementarity of the nucleotide sequences of YWHAE BINDING SITE, designated SEQ ID:1329, to the nucleotide sequence of VGAM101 RNA, herein designated VGAM RNA, also designated SEQ ID:436.

Another function of VGAM101 is therefore inhibition of Tyrosine 3-monooxygenase/tryptophan 5-monooxygenase Activation Protein, Epsilon Polypeptide (YWHAE, Accession NM_{—}006761), a gene which binds to cdc25 and may facilitate cdc25 interaction with Raf-1 in vivo. Accordingly, utilities of VGAM101 include diagnosis, prevention and treatment of diseases and clinical conditions associated with YWHAE. The function of YWHAE has been established by previous studies. Using a yeast 2-hybrid screen to probe a HeLa cell library with CDC25A (OMIM Ref. No. 116947) and CDC25B (OMIM Ref. No. 116949) as bait, Conklin et al. (1995) isolated cDNAs encoding YWHAB (OMIM Ref. No. 601289), which they called 14-3-3-beta, and YWHAE, which they called 14-3-3-epsilon. YWHAE encodes a deduced 260-amino acid protein that is 100% identical to the mouse sequence. Both 14-3-3 proteins interacted with either CDC protein but did not affect their phosphatase activities. Like YWHAB, YWHAE interacted with RAF1 (OMIM Ref. No. 164760) but not RAS (OMIM Ref. No. 190020) in yeast 2-hybrid screens and may facilitate the association of CDC25 with RAF1. The binding of insulin (OMIM Ref. No. 176730) to its receptor induces the phosphorylation of the cytosolic substrates IRS1 (OMIM Ref. No. 147545) and IRS2 (OMIM Ref. No. 600797), which associate with several Src homology-2 (SH2) domain-containing proteins. To identify unique IRS1-binding proteins, Ogihara et al. (1997) screened a human heart cDNA expression library with recombinant IRS1. They obtained 2 isoforms of the 14-3-3 protein family, 14-3-3-zeta (YWHAZ; 601288) and -epsilon. 14-3-3 protein has been shown to associate with IRS1 in L6 myotubes, HepG2 hepatoma cells, Chinese hamster ovary cells, and bovine brain tissue. IRS2, a protein structurally similar to IRS1, was also shown to form a complex with 14-3-3 protein using a baculovirus expression system. The amount of 14-3-3 protein associated with IRS1 was not affected by insulin stimulation but was increased significantly by treatment with okadaic acid, a potent serine/threonine phosphatase inhibitor. The authors identified a putative 14-3-3 protein-binding site within the phosphotyrosine-binding (PTB) domain of IRS1. Ogihara et al. (1997) suggested that the association with 14-3-3 protein may play a role in the regulation of insulin sensitivity by interrupting the association between the insulin receptor and IRS1.

Full details of the abovementioned studies are described in the following publications, the disclosure of which are hereby incorporated by reference:
Conklin, D. S.; Galaktionov, K.; Beach, D.: 14-3-3 proteins associate with cdc25 phosphatases. Proc. Nat. Acad. Sci. 92: 7892-7896, 1995.; and
Ogihara, T.; Isobe, T.; Ichimura, T.; Taoka, M.; Funaki, M.; Sakoda, H.; Onishi, Y.; Inukai, K.; Anai, M.; Fukushima, Y.; Kikuchi, M.; Yazaki, Y.; Oka, Y.; Asano, T.: 14-3-3 protein bin.

Further studies establishing the function and utilities of YWHAE are found in John Hopkins OMIM database record ID 605066, and in sited publications numbered 1577-1578, 2285-158 and 1001 listed in the bibliography section hereinbelow, which are also hereby incorporated by reference. A Kinase (PRKA) Anchor Protein (yotiao) 9 (AKAP9, Accession NM_{—}005751) is another VGAM101 host target gene. AKAP9 BINDING SITE is HOST TARGET binding site found in the 3′ untranslated region of mRNA encoded by AKAP9, corresponding to a HOST TARGET binding site such as BINDING SITE I, BINDING SITE II or BINDING SITE III. Table 2 illustrates the complementarity of the nucleotide sequences of AKAP9 BINDING SITE, designated SEQ ID:1242, to the nucleotide sequence of VGAM101 RNA, herein designated VGAM RNA, also designated SEQ ID:436.

Another function of VGAM101 is therefore inhibition of A Kinase (PRKA) Anchor Protein (yotiao) 9 (AKAP9, Accession NM_{—}005751). Accordingly, utilities of VGAM101 include diagnosis, prevention and treatment of diseases and clinical conditions associated with AKAP9. LOC166824 (Accession XM_{—}094119) is another VGAM101 host target gene. LOC166824 BINDING SITE is HOST TARGET binding site found in the 3′ untranslated region of mRNA encoded by LOC166824, corresponding to a HOST TARGET binding site such as BINDING SITE I, BINDING SITE II or BINDING SITE III. Table 2 illustrates the complementarity of the nucleotide sequences of LOC166824 BINDING SITE, designated SEQ ID:3259, to the nucleotide sequence of VGAM101 RNA, herein designated VGAM RNA, also designated SEQ ID:436.

Another function of VGAM101 is therefore inhibition of LOC166824 (Accession XM_{—}094119). Accordingly, utilities of VGAM101 include diagnosis, prevention and treatment of diseases and clinical conditions associated with LOC166824. LOC254835 (Accession XM_{—}173056) is another VGAM101 host target gene. LOC254835 BINDING SITE is HOST TARGET binding site found in the 3′ untranslated region of mRNA encoded by LOC254835, corresponding to a HOST TARGET binding site such as BINDING SITE I, BINDING SITE II or BINDING SITE III. Table 2 illustrates the complementarity of the nucleotide sequences of LOC254835 BINDING SITE, designated SEQ ID:3722, to the nucleotide sequence of VGAM101 RNA, herein designated VGAM RNA, also designated SEQ ID:436.

Another function of VGAM101 is therefore inhibition of LOC254835 (Accession XM_{—}173056). Accordingly, utilities of VGAM101 include diagnosis, prevention and treatment of diseases and clinical conditions associated with LOC254835. FIG. 1 further provides a conceptual description of a novel bioinformatically detected viral gene of the present invention, referred to here as Viral Genomic Address Messenger 102 (VGAM102) viral gene, which modulates expression of respective host target genes thereof, the function and utility of which host target genes is known in the art.

VGAM102 is a novel bioinformatically detected regulatory, non protein coding, viral micro RNA (miRNA) gene. The method by which VGAM102 was detected is described hereinabove with reference to FIGS. 1-8.

VGAM102 gene, herein designated VGAM GENE, is a viral gene contained in the genome of Vaccinia Virus. VGAM102 host target gene, herein designated VGAM HOST TARGET GENE, is a human gene contained in the human genome.

VGAM102 gene encodes a VGAM102 precursor RNA, herein designated VGAM PRECURSOR RNA. Similar to other miRNA genes, and unlike most ordinary genes, VGAM102 precursor RNA does not encode a protein. A nucleotide sequence identical or highly similar to the nucleotide sequence of VGAM102 precursor RNA is designated SEQ ID:88, and is provided hereinbelow with reference to the sequence listing part. Nucleotide sequence SEQ ID:88 is located at position 41122 relative to the genome of Vaccinia Virus.

VGAM102 precursor RNA folds onto itself, forming VGAM102 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM102 folded precursor RNA into VGAM102 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM102 RNA is designated SEQ ID:437, and is provided hereinbelow with reference to the sequence listing part.

VGAM102 host target gene, herein designated VGAM HOST TARGET GENE, encodes a corresponding messenger RNA, VGAM102 host target RNA, herein designated VGAM HOST TARGET RNA. VGAM102 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM102 RNA, herein designated VGAM RNA, binds complementarily to one or more host target binding sites located in untranslated regions of VGAM102 host target RNA, herein designated VGAM HOST TARGET RNA. This complementary binding is due to the fact that the nucleotide sequence of VGAM102 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM102 RNA, herein designated VGAM RNA, may have a different number of host target binding sites in untranslated regions of a VGAM102 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM102 RNA, herein designated VGAM RNA, to host target binding sites on VGAM102 host target RNA, herein designated VGAM HOST TARGET RNA, such as BINDING SITE I, BINDING SITE II and BINDING SITE III, inhibits translation of VGAM102 host target RNA into VGAM102 host target protein, herein designated VGAM HOST TARGET PROTEIN. VGAM host target protein is therefore outlined by a broken line.

It is appreciated that VGAM102 host target gene, herein designated VGAM HOST TARGET GENE, in fact represents a plurality of VGAM102 host target genes. The mRNA of each one of this plurality of VGAM102 host target genes comprises one or more host target binding sites, each having a nucleotide sequence which is at least partly complementary to VGAM102 RNA, herein designated VGAM RNA, and which when bound by VGAM102 RNA causes inhibition of translation of respective one or more VGAM102 host target proteins.

It is further appreciated by one skilled in the art that the mode of translational inhibition illustrated by FIG. 1 with specific reference to translational inhibition exerted by VGAM102 gene, herein designated VGAM GENE, on one or more VGAM102 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM102 is inhibition of expression of host target genes, as part of a novel viral mechanism of attacking a host. Accordingly, utilities of VGAM102 include diagnosis, prevention and treatment of viral infection by Vaccinia Virus. Specific functions, and accordingly utilities, of VGAM102 correlate with, and may be deduced from, the identity of the host target genes which VGAM102 binds and inhibits, and the function of these host target genes, as elaborated hereinbelow.

Nucleotide sequences of the VGAM102 precursor RNA, herein designated VGAM PRECURSOR RNA, and of the ‘diced’ VGAM102 RNA, herein designated VGAM RNA, and a schematic representation of the secondary folding of VGAM102 folded precursor RNA, herein designated VGAM FOLDED PRECURSOR RNA, of VGAM102 are further described hereinbelow with reference to Table 1.

Nucleotide sequences of host target binding sites, such as BINDING SITE-I, BINDING SITE-II and BINDING SITE-III of FIG. 1, found on VGAM102 host target RNA, and schematic representation of the complementarity of each of these host target binding sites to VGAM102 RNA, herein designated VGAM RNA, are described hereinbelow with reference to Table 2.

As mentioned hereinabove with reference to FIG. 1, a function of VGAM102 gene, herein designated VGAM is inhibition of expression of VGAM102 target genes. It is appreciated that specific functions, and accordingly utilities, of VGAM102 correlate with, and may be deduced from, the identity of the target genes which VGAM102 binds and inhibits, and the function of these target genes, as elaborated hereinbelow.

PNPASE (Accession XM_{—}048088) is a VGAM102 host target gene. PNPASE BINDING SITE is HOST TARGET binding site found in the 3′ untranslated region of mRNA encoded by PNPASE, corresponding to a HOST TARGET binding site such as BINDING SITE I, BINDING SITE II or BINDING SITE III. Table 2 illustrates the complementarity of the nucleotide sequences of PNPASE BINDING SITE, designated SEQ ID:2900, to the nucleotide sequence of VGAM102 RNA, herein designated VGAM RNA, also designated SEQ ID:437.

A function of VGAM102 is therefore inhibition of PNPASE (Accession XM_{—}048088). Accordingly, utilities of VGAM102 include diagnosis, prevention and treatment of diseases and clinical conditions associated with PNPASE. SEC24 Related Gene Family, Member D (S. cerevisiae) (SEC24D, Accession NM_{—}014822) is another VGAM102 host target gene. SEC24D BINDING SITE is HOST TARGET binding site found in the 3′ untranslated region of mRNA encoded by SEC24D, corresponding to a HOST TARGET binding site such as BINDING SITE I, BINDING SITE II or BINDING SITE III. Table 2 illustrates the complementarity of the nucleotide sequences of SEC24D BINDING SITE, designated SEQ ID:1559, to the nucleotide sequence of VGAM102 RNA, herein designated VGAM RNA, also designated SEQ ID:437.

Another function of VGAM102 is therefore inhibition of SEC24 Related Gene Family, Member D (S. cerevisiae) (SEC24D, Accession NM_{—}014822). Accordingly, utilities of VGAM102 include diagnosis, prevention and treatment of diseases and clinical conditions associated with SEC24D. LOC131000 (Accession XM_{—}067145) is another VGAM102 host target gene. LOC131000 BINDING SITE is HOST TARGET binding site found in the 3′ untranslated region of mRNA encoded by LOC131000, corresponding to a HOST TARGET binding site such as BINDING SITE I, BINDING SITE II or BINDING SITE III. Table 2 illustrates the complementarity of the nucleotide sequences of LOC131000 BINDING SITE, designated SEQ ID:3037, to the nucleotide sequence of VGAM102 RNA, herein designated VGAM RNA, also designated SEQ ID:437.

Another function of VGAM102 is therefore inhibition of LOC131000 (Accession XM_{—}067145). Accordingly, utilities of VGAM102 include diagnosis, prevention and treatment of diseases and clinical conditions associated with LOC131000. LOC151658 (Accession XM_{—}098103) is another VGAM102 host target gene. LOC151658 BINDING SITE is HOST TARGET binding site found in the 5′ untranslated region of mRNA encoded by LOC151658, corresponding to a HOST TARGET binding site such as BINDING SITE I, BINDING SITE II or BINDING SITE III. Table 2 illustrates the complementarity of the nucleotide sequences of LOC151658 BINDING SITE, designated SEQ ID:3349, to the nucleotide sequence of VGAM102 RNA, herein designated VGAM RNA, also designated SEQ ID:437.

Another function of VGAM102 is therefore inhibition of LOC151658 (Accession XM_{—}098103). Accordingly, utilities of VGAM102 include diagnosis, prevention and treatment of diseases and clinical conditions associated with LOC151658. LOC257428 (Accession XM_{—}168584) is another VGAM102 host target gene. LOC257428 BINDING SITE is HOST TARGET binding site found in the 3′ untranslated region of mRNA encoded by LOC257428, corresponding to a HOST TARGET binding site such as BINDING SITE I, BINDING SITE II or BINDING SITE III. Table 2 illustrates the complementarity of the nucleotide sequences of LOC257428 BINDING SITE, designated SEQ ID:3649, to the nucleotide sequence of VGAM102 RNA, herein designated VGAM RNA, also designated SEQ ID:437.

Another function of VGAM102 is therefore inhibition of LOC257428 (Accession XM_{—}168584). Accordingly, utilities of VGAM102 include diagnosis, prevention and treatment of diseases and clinical conditions associated with LOC257428. FIG. 1 further provides a conceptual description of a novel bioinformatically detected viral gene of the present invention, referred to here as Viral Genomic Address Messenger 103 (VGAM103) viral gene, which modulates expression of respective host target genes thereof, the function and utility of which host target genes is known in the art.

VGAM103 is a novel bioinformatically detected regulatory, non protein coding, viral micro RNA (miRNA) gene. The method by which VGAM103 was detected is described hereinabove with reference to FIGS. 1-8.

VGAM103 gene, herein designated VGAM GENE, is a viral gene contained in the genome of Vaccinia Virus. VGAM103 host target gene, herein designated VGAM HOST TARGET GENE, is a human gene contained in the human genome.

VGAM103 gene encodes a VGAM103 precursor RNA, herein designated VGAM PRECURSOR RNA. Similar to other miRNA genes, and unlike most ordinary genes, VGAM103 precursor RNA does not encode a protein. A nucleotide sequence identical or highly similar to the nucleotide sequence of VGAM103 precursor RNA is designated SEQ ID:89, and is provided hereinbelow with reference to the sequence listing part. Nucleotide sequence SEQ ID:89 is located at position 42559 relative to the genome of Vaccinia Virus.

VGAM103 precursor RNA folds onto itself, forming VGAM103 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM103 folded precursor RNA into VGAM103 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM103 RNA is designated SEQ ID:438, and is provided hereinbelow with reference to the sequence listing part.

VGAM103 host target gene, herein designated VGAM HOST TARGET GENE, encodes a corresponding messenger RNA, VGAM103 host target RNA, herein designated VGAM HOST TARGET RNA. VGAM103 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM103 RNA, herein designated VGAM RNA, binds complementarily to one or more host target binding sites located in untranslated regions of VGAM103 host target RNA, herein designated VGAM HOST TARGET RNA. This complementary binding is due to the fact that the nucleotide sequence of VGAM103 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM103 RNA, herein designated VGAM RNA, may have a different number of host target binding sites in untranslated regions of a VGAM103 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM103 RNA, herein designated VGAM RNA, to host target binding sites on VGAM103 host target RNA, herein designated VGAM HOST TARGET RNA, such as BINDING SITE I, BINDING SITE II and BINDING SITE III, inhibits translation of VGAM103 host target RNA into VGAM103 host target protein, herein designated VGAM HOST TARGET PROTEIN. VGAM host target protein is therefore outlined by a broken line.

It is appreciated that VGAM103 host target gene, herein designated VGAM HOST TARGET GENE, in fact represents a plurality of VGAM103 host target genes. The mRNA of each one of this plurality of VGAM103 host target genes comprises one or more host target binding sites, each having a nucleotide sequence which is at least partly complementary to VGAM103 RNA, herein designated VGAM RNA, and which when bound by VGAM103 RNA causes inhibition of translation of respective one or more VGAM103 host target proteins.

It is further appreciated by one skilled in the art that the mode of translational inhibition illustrated by FIG. 1 with specific reference to translational inhibition exerted by VGAM103 gene, herein designated VGAM GENE, on one or more VGAM103 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM103 is inhibition of expression of host target genes, as part of a novel viral mechanism of attacking a host. Accordingly, utilities of VGAM103 include diagnosis, prevention and treatment of viral infection by Vaccinia Virus. Specific functions, and accordingly utilities, of VGAM103 correlate with, and may be deduced from, the identity of the host target genes which VGAM103 binds and inhibits, and the function of these host target genes, as elaborated hereinbelow.

Nucleotide sequences of the VGAM103 precursor RNA, herein designated VGAM PRECURSOR RNA, and of the ‘diced’ VGAM103 RNA, herein designated VGAM RNA, and a schematic representation of the secondary folding of VGAM103 folded precursor RNA, herein designated VGAM FOLDED PRECURSOR RNA, of VGAM103 are further described hereinbelow with reference to Table 1.

Nucleotide sequences of host target binding sites, such as BINDING SITE-I, BINDING SITE-II and BINDING SITE-III of FIG. 1, found on VGAM103 host target RNA, and schematic representation of the complementarity of each of these host target binding sites to VGAM103 RNA, herein designated VGAM RNA, are described hereinbelow with reference to Table 2.

As mentioned hereinabove with reference to FIG. 1, a function of VGAM103 gene, herein designated VGAM is inhibition of expression of VGAM103 target genes. It is appreciated that specific functions, and accordingly utilities, of VGAM103 correlate with, and may be deduced from, the identity of the target genes which VGAM103 binds and inhibits, and the function of these target genes, as elaborated hereinbelow.

Caspase Recruitment Domain Family, Member 15 (CARD15, Accession NM_{—}022162) is a VGAM103 host target gene. CARD15 BINDING SITE is HOST TARGET binding site found in the 3′ untranslated region of mRNA encoded by CARD15, corresponding to a HOST TARGET binding site such as BINDING SITE I, BINDING SITE II or BINDING SITE III. Table 2 illustrates the complementarity of the nucleotide sequences of CARD15 BINDING SITE, designated SEQ ID: 1983, to the nucleotide sequence of VGAM103 RNA, herein designated VGAM RNA, also designated SEQ ID:438.

A function of VGAM103 is therefore inhibition of Caspase Recruitment Domain Family, Member 15 (CARD15, Accession NM_{—}022162), a gene which serves as an intracellular receptor for bacterial products in monocytes and transduces signals leading to NFKB activation. Accordingly, utilities of VGAM103 include diagnosis, prevention and treatment of diseases and clinical conditions associated with CARD15. The function of CARD15 has been established by previous studies. By searching a genomic database for NOD1 homologs, followed by 5-prime RACE and RT-PCR, Ogura et al. (2001) obtained cDNAs encoding NOD2. Sequence analysis predicted that the 1,040-amino acid NOD2 protein, which is 34% identical to NOD1, contains 2 N-terminal CARDs fused to a central NBD domain followed by 10 tandem LRRs. Ogura et al. (2001) also identified a NOD2 variant encoding a 1,013-amino acid protein, which they called NOD2B. Northern blot analysis detected 7.0- and 5.5-kb NOD2 transcripts in peripheral blood leukocytes, with little or no expression found in other tissues. RT-PCR analysis revealed expression primarily in monocytes. In contrast, NOD1 and APAF1 are broadly expressed. Expression of NOD2 or NOD2B resulted in NFKB activation, and mutants lacking the LRRs had enhanced NFKB activation. The authors determined that both intact CARD domains are necessary and sufficient for IKK-gamma (IKBKG; 300248)- and RICK (RIPK2; 603455)-dependent NFKB activation. Coimmunoprecipitation analysis showed that the CARD domain of RICK interacts with the CARD domains of NOD2. Lesage et al. (2002) reported the mutational analysis of the CARD15 gene in 453 patients with Crohn disease, including 166 sporadic and 287 familial cases, 159 patients with ulcerative colitis (OMIM Ref. No. 191390), and 103 healthy control subjects. Although no mutations were found to be associated with ulcerative colitis, 50% of patients with Crohn disease carried at least 1 potential disease-causing mutation, including 17% who had a double mutation. There were 27 rare additional mutations. Each of 3 polymorphisms (R702W, G908R, and 1007fs) were confirmed to be intermittently associated with susceptibility to Crohn disease. These 3 main variants represented 32%, 18%, and 31%, respectively, of the total Crohn disease mutations, whereas the total of the 27 rare mutations represented 19% of disease-causing mutations. Altogether, 93% of the mutations were located in the distal third of the gene. These observations confirmed the gene-dosage effect in Crohn disease. Patients with double-dose mutations were characterized by a younger age at onset, a more frequent stricturing phenotype, and a less frequent colonic involvement than were seen in those patients who had no mutation. The severity of the disease and extraintestinal manifestations were not different for any of the CARD15 genotypes. The proportion of familial and sporadic cases and the proportion of patients with smoking habits were similar in the groups of Crohn disease patients with or without mutation.

Full details of the abovementioned studies are described in the following publications, the disclosure of which are hereby incorporated by reference:
Ogura, Y.; Inohara, N.; Benito, A.; Chen, F. F.; Yamaoka, S.; Nunez, G.: Nod2, a Nod1/Apaf-1 family member that is restricted to monocytes and activates NF-kappa-B. J. Biol. Chem. 276: 4812-4818, 2001.; and
Vermeire, S.; Wild, G.; Kocher, K.; Cousineau, J.; Dufresne, L.; Bitton, A.; Langelier, D.; Pare, P.; Lapointe, G.; Cohen, A.; Daly, M. J.; Rioux, J. D.: CARD15 genetic variation in a.

Further studies establishing the function and utilities of CARD15 are found in John Hopkins OMIM database record ID 605956, and in sited publications numbered 1568-1569, 1573, 2284-157 and 1574-1576 listed in the bibliography section hereinbelow, which are also hereby incorporated by reference. Chloride Channel 4 (CLCN4, Accession NM_{—}001830) is another VGAM103 host target gene. CLCN4 BINDING SITE is HOST TARGET binding site found in the 3′ untranslated region of mRNA encoded by CLCN4, corresponding to a HOST TARGET binding site such as BINDING SITE I, BINDING SITE II or BINDING SITE III. Table 2 illustrates the complementarity of the nucleotide sequences of CLCN4 BINDING SITE, designated SEQ ID:861, to the nucleotide sequence of VGAM103 RNA, herein designated VGAM RNA, also designated SEQ ID:438.

Another function of VGAM103 is therefore inhibition of Chloride Channel 4 (CLCN4, Accession NM_{—}001830), a gene which is regulation of cell volume; membrane potential stabilization, signal transduction and transepithelial transport. Accordingly, utilities of VGAM103 include diagnosis, prevention and treatment of diseases and clinical conditions associated with CLCN4. The function of CLCN4 has been established by previous studies. In the course of constructing a comprehensive transcript map of the Xp22.3 region, van Slegtenhorst et al. (1994) identified an evolutionarily conserved CpG island and cloned the corresponding gene. The predicted 760-amino acid protein contained 12 hydrophobic domains and shared sequence and structural similarities with all the previously isolated members of the family of voltage-gated chloride channels. The gene, termed CLCN4, for chloride channel 4, contained at least 10 exons spanning 60 to 80 kb. In contrast with most genes isolated from the Xp22.3 region, CLCN4 did not share homology with the Y chromosome, but was conserved in mouse and hamster. Expression studies demonstrated a 7.5-kb transcript that is particularly abundant in skeletal muscle and also detectable in brain and heart. Thus, this gene encodes a newly identified voltage-gated chloride channel. Rugarli et al. (1995) found that in the wild Mediterranean mouse Mus spretus, the Cln4 gene maps to the X chromosome as it does in the human; however, in the inbred strain of laboratory mouse C57BL/6J, they found that it maps to chromosome 7. Findings indicated that a recent evolutionary rearrangement occurred in the mouse sex chromosomes very close to the pseudoautosomal region (PAR). The data were considered molecular evidence for a major divergence near the pseudoautosomal region consistent with the hypothesis that hybrid sterility in these species results from abnormal pairing of sex chromosomes during male meiosis. They found that Cln4 is the closest cloned gene to the M. spretus pseudoautosomal region and the most distal locus displaying a conserved position between the human and this mouse locus. The X-inactivation status of the locus in M. spretus was demonstrated by the finding that in F1 females from a cross between M. spretus and an inbred-derived mouse carrying the t(X;16) 16H balanced translocation, it was always the normal M. spretus X chromosome that was inactive in adult tissues. This completely skewed X inactivation provided a system for assaying expression of genes from the inactive X chromosome once the parental alleles could be distinguished. Palmer et al. (1995) likewise found what they referred to as ‘contravention of Ohno's law’ in the course of mapping a cDNA mouse Cln4 in an interspecific backcross. This was the first example of a gene unique to the X chromosome in 1 eutherian species but autosomal in another. The consequence of this chromosomal rearrangement was that the gene was lost by mendelian segregation in a subset of the male progeny of a (C57BL/6×Mus spretus)×Mus spretus backcross.

Full details of the abovementioned studies are described in the following publications, the disclosure of which are hereby incorporated by reference:
Rugarli, E. I.; Adler, D. A.; Borsani, G.; Tsuchiya, K.; Franco, B.; Hauge, X.; Disteche, C.; Chapman, V.; Ballabio, A.: Different chromosomal localization of the Clcn4 gene in Mus spretus and C57BL/6J mice. Nature Genet. 10: 466-471, 1995.; and
van Slegtenhorst, M. A.; Bassi, M. T.; Borsani, G.; Wapenaar, M. C.; Ferrero, G. B.; de Conciliis, L.; Rugarli, E. I.; Grillo, A.; Franco, B.; Zoghbi, H. Y.; Ballabio, A.: A gene from.

Further studies establishing the function and utilities of CLCN4 are found in John Hopkins OMIM database record ID 302910, and in sited publications numbered 248 and 2530-2537 listed in the bibliography section hereinbelow, which are also hereby incorporated by reference. Fibroblast Growth Factor 2 (basic) (FGF2, Accession NM_{—}002006) is another VGAM103 host target gene. FGF2 BINDING SITE is HOST TARGET binding site found in the 3′ untranslated region of mRNA encoded by FGF2, corresponding to a HOST TARGET binding site such as BINDING SITE I, BINDING SITE II or BINDING SITE III. Table 2 illustrates the complementarity of the nucleotide sequences of FGF2 BINDING SITE, designated SEQ ID:885, to the nucleotide sequence of VGAM103 RNA, herein designated VGAM RNA, also designated SEQ ID:438.

Another function of VGAM103 is therefore inhibition of Fibroblast Growth Factor 2 (basic) (FGF2, Accession NM_{—}002006), a gene which Basic fibroblast growth factor 2. Accordingly, utilities of VGAM103 include diagnosis, prevention and treatment of diseases and clinical conditions associated with FGF2. The function of FGF2 and its association with various diseases and clinical conditions, has been established by previous studies, as described hereinabove with reference to VGAM30. Zinc Finger Protein 135 (clone pHZ-17) (ZNF135, Accession NM_{—}003436) is another VGAM103 host target gene. ZNF135 BINDING SITE is HOST TARGET binding site found in the 3′ untranslated region of mRNA encoded by ZNF135, corresponding to a HOST TARGET binding site such as BINDING SITE I, BINDING SITE II or BINDING SITE III. Table 2 illustrates the complementarity of the nucleotide sequences of ZNF135 BINDING SITE, designated SEQ ID: 1022, to the nucleotide sequence of VGAM103 RNA, herein designated VGAM RNA, also designated SEQ ID:438.

Another function of VGAM103 is therefore inhibition of Zinc Finger Protein 135 (clone pHZ-17) (ZNF135, Accession NM_{—}003436). Accordingly, utilities of VGAM103 include diagnosis, prevention and treatment of diseases and clinical conditions associated with ZNF135. SEC15B (Accession XM_{—}039570) is another VGAM103 host target gene. SEC15B BINDING SITE is HOST TARGET binding site found in the 3′ untranslated region of mRNA encoded by SEC15B, corresponding to a HOST TARGET binding site such as BINDING SITE I, BINDING SITE II or BINDING SITE III. Table 2 illustrates the complementarity of the nucleotide sequences of SEC15B BINDING SITE, designated SEQ ID:2757, to the nucleotide sequence of VGAM103 RNA, herein designated VGAM RNA, also designated SEQ ID:438.

Another function of VGAM103 is therefore inhibition of SEC15B (Accession XM_{—}039570). Accordingly, utilities of VGAM103 include diagnosis, prevention and treatment of diseases and clinical conditions associated with SEC15B. Zinc Finger Protein 334 (ZNF334, Accession NM_{—}018102) is another VGAM103 host target gene. ZNF334 BINDING SITE is HOST TARGET binding site found in the 3′ untranslated region of mRNA encoded by ZNF334, corresponding to a HOST TARGET binding site such as BINDING SITE I, BINDING SITE II or BINDING SITE III. Table 2 illustrates the complementarity of the nucleotide sequences of ZNF334 BINDING SITE, designated SEQ ID:1780, to the nucleotide sequence of VGAM103 RNA, herein designated VGAM RNA, also designated SEQ ID:438.

Another function of VGAM103 is therefore inhibition of Zinc Finger Protein 334 (ZNF334, Accession NM_{—}018102). Accordingly, utilities of VGAM103 include diagnosis, prevention and treatment of diseases and clinical conditions associated with ZNF334. LOC158954 (Accession XM_{—}017340) is another VGAM103 host target gene. LOC158954 BINDING SITE is HOST TARGET binding site found in the 3′ untranslated region of mRNA encoded by LOC158954, corresponding to a HOST TARGET binding site such as BINDING SITE I, BINDING SITE II or BINDING SITE III. Table 2 illustrates the complementarity of the nucleotide sequences of LOC158954 BINDING SITE, designated SEQ ID:2567, to the nucleotide sequence of VGAM103 RNA, herein designated VGAM RNA, also designated SEQ ID:438.

Another function of VGAM103 is therefore inhibition of LOC158954 (Accession XM_{—}017340). Accordingly, utilities of VGAM103 include diagnosis, prevention and treatment of diseases and clinical conditions associated with LOC158954. LOC219686 (Accession XM_{—}165544) is another VGAM103 host target gene. LOC219686 BINDING SITE is HOST TARGET binding site found in the 3′ untranslated region of mRNA encoded by LOC219686, corresponding to a HOST TARGET binding site such as BINDING SITE I, BINDING SITE II or BINDING SITE III. Table 2 illustrates the complementarity of the nucleotide sequences of LOC219686 BINDING SITE, designated SEQ ID:3500, to the nucleotide sequence of VGAM103 RNA, herein designated VGAM RNA, also designated SEQ ID:438.

Another function of VGAM103 is therefore inhibition of LOC219686 (Accession XM_{—}165544). Accordingly, utilities of VGAM103 include diagnosis, prevention and treatment of diseases and clinical conditions associated with LOC219686. LOC221510 (Accession XM_{—}165807) is another VGAM103 host target gene. LOC221510 BINDING SITE is HOST TARGET binding site found in the 3′ untranslated region of mRNA encoded by LOC221510, corresponding to a HOST TARGET binding site such as BINDING SITE I, BINDING SITE II or BINDING SITE III. Table 2 illustrates the complementarity of the nucleotide sequences of LOC221510 BINDING SITE, designated SEQ ID:3508, to the nucleotide sequence of VGAM103 RNA, herein designated VGAM RNA, also designated SEQ ID:438.

Another function of VGAM103 is therefore inhibition of LOC221510 (Accession XM_{—}165807). Accordingly, utilities of VGAM103 include diagnosis, prevention and treatment of diseases and clinical conditions associated with LOC221510. FIG. 1 further provides a conceptual description of a novel bioinformatically detected viral gene of the present invention, referred to here as Viral Genomic Address Messenger 104 (VGAM104) viral gene, which modulates expression of respective host target genes thereof, the function and utility of which host target genes is known in the art.

VGAM104 is a novel bioinformatically detected regulatory, non protein coding, viral micro RNA (miRNA) gene. The method by which VGAM104 was detected is described hereinabove with reference to FIGS. 1-8.

VGAM104 gene, herein designated VGAM GENE, is a viral gene contained in the genome of Vaccinia Virus. VGAM104 host target gene, herein designated VGAM HOST TARGET GENE, is a human gene contained in the human genome.

VGAM104 gene encodes a VGAM104 precursor RNA, herein designated VGAM PRECURSOR RNA. Similar to other miRNA genes, and unlike most ordinary genes, VGAM104 precursor RNA does not encode a protein. A nucleotide sequence identical or highly similar to the nucleotide sequence of VGAM104 precursor RNA is designated SEQ ID:90, and is provided hereinbelow with reference to the sequence listing part. Nucleotide sequence SEQ ID:90 is located at position 44309 relative to the genome of Vaccinia Virus.

VGAM104 precursor RNA folds onto itself, forming VGAM104 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM104 folded precursor RNA into VGAM104 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM104 RNA is designated SEQ ID:439, and is provided hereinbelow with reference to the sequence listing part.

VGAM104 host target gene, herein designated VGAM HOST TARGET GENE, encodes a corresponding messenger RNA, VGAM104 host target RNA, herein designated VGAM HOST TARGET RNA. VGAM104 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM104 RNA, herein designated VGAM RNA, binds complementarily to one or more host target binding sites located in untranslated regions of VGAM104 host target RNA, herein designated VGAM HOST TARGET RNA. This complementary binding is due to the fact that the nucleotide sequence of VGAM104 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM104 RNA, herein designated VGAM RNA, may have a different number of host target binding sites in untranslated regions of a VGAM104 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM104 RNA, herein designated VGAM RNA, to host target binding sites on VGAM104 host target RNA, herein designated VGAM HOST TARGET RNA, such as BINDING SITE I, BINDING SITE II and BINDING SITE III, inhibits translation of VGAM104 host target RNA into VGAM104 host target protein, herein designated VGAM HOST TARGET PROTEIN. VGAM host target protein is therefore outlined by a broken line.

It is appreciated that VGAM104 host target gene, herein designated VGAM HOST TARGET GENE, in fact represents a plurality of VGAM104 host target genes. The mRNA of each one of this plurality of VGAM104 host target genes comprises one or more host target binding sites, each having a nucleotide sequence which is at least partly complementary to VGAM104 RNA, herein designated VGAM RNA, and which when bound by VGAM104 RNA causes inhibition of translation of respective one or more VGAM104 host target proteins.

It is further appreciated by one skilled in the art that the mode of translational inhibition illustrated by FIG. 1 with specific reference to translational inhibition exerted by VGAM104 gene, herein designated VGAM GENE, on one or more VGAM104 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM104 is inhibition of expression of host target genes, as part of a novel viral mechanism of attacking a host. Accordingly, utilities of VGAM104 include diagnosis, prevention and treatment of viral infection by Vaccinia Virus. Specific functions, and accordingly utilities, of VGAM104 correlate with, and may be deduced from, the identity of the host target genes which VGAM104 binds and inhibits, and the function of these host target genes, as elaborated hereinbelow.

Nucleotide sequences of the VGAM104 precursor RNA, herein designated VGAM PRECURSOR RNA, and of the ‘diced’ VGAM104 RNA, herein designated VGAM RNA, and a schematic representation of the secondary folding of VGAM104 folded precursor RNA, herein designated VGAM FOLDED PRECURSOR RNA, of VGAM104 are further described hereinbelow with reference to Table 1.

Nucleotide sequences of host target binding sites, such as BINDING SITE-I, BINDING SITE-II and BINDING SITE-III of FIG. 1, found on VGAM104 host target RNA, and schematic representation of the complementarity of each of these host target binding sites to VGAM104 RNA, herein designated VGAM RNA, are described hereinbelow with reference to Table 2.

As mentioned hereinabove with reference to FIG. 1, a function of VGAM104 gene, herein designated VGAM is inhibition of expression of VGAM104 target genes. It is appreciated that specific functions, and accordingly utilities, of VGAM104 correlate with, and may be deduced from, the identity of the target genes which VGAM104 binds and inhibits, and the function of these target genes, as elaborated hereinbelow.

Phospholamban (PLN, Accession NM_{—}002667) is a VGAM104 host target gene. PLN BINDING SITE is HOST TARGET binding site found in the 3′ untranslated region of mRNA encoded by PLN, corresponding to a HOST TARGET binding site such as BINDING SITE I, BINDING SITE II or BINDING SITE III. Table 2 illustrates the complementarity of the nucleotide sequences of PLN BINDING SITE, designated SEQ ID:947, to the nucleotide sequence of VGAM104 RNA, herein designated VGAM RNA, also designated SEQ ID:439.

A function of VGAM104 is therefore inhibition of Phospholamban (PLN, Accession NM_{—}002667), a gene which regulates the activity of the calcium pump of cardiac sarcoplasmic reticulum. Accordingly, utilities of VGAM104 include diagnosis, prevention and treatment of diseases and clinical conditions associated with PLN. The function of PLN has been established by previous studies. Fujii et al. (1991) isolated and characterized genomic DNA clones encoding rabbit phospholamban. The phospholamban gene of 13.2 kb contained only one 10.5-kb intron which separated exonic sequences located in the 5-prime untranslated region. Phospholamban, through modulation of sarcoplasmic reticulum calcium-ATPase activity, is a key regulator of cardiac diastolic function. Alterations in phospholamban expression may define parameters of muscle relaxation. McTiernan et al. (1999) observed that human ventricle and quadriceps displayed high levels of phospholamban transcripts and proteins, with markedly lower expression observed in smooth muscles, while the right atrium also expressed low levels of phospholamban. The structure of the human phospholamban gene closely resembles that reported for chicken, rabbit, rat, and mouse. Comparison of the human to other mammalian phospholamban genes indicated a marked conservation of sequence for at least 217 bp upstream of the transcription start site.

Full details of the abovementioned studies are described in the following publications, the disclosure of which are hereby incorporated by reference:
Fujii, J.; Zarain-Herzberg, A.; Willard, H. F.; Tada, M.; MacLennan, D. H.: Structure of the rabbit phospholamban gene, cloning of the human cDNA, and assignment of the gene to human chromosome 6. J. Biol. Chem. 266: 11669-11675, 1991.; and
McTiernan, C. F.; Frye, C. S.; Lemster, B. H.; Kinder, E. A.; Ogletree-Hughes, M. L.; Moravec, C. S.; Feldman, A. M.: The human phospholamban gene: structure and expression. J. Molec. Cell.

Further studies establishing the function and utilities of PLN are found in John Hopkins OMIM database record ID 172405, and in sited publications numbered 2485-2487 listed in the bibliography section hereinbelow, which are also hereby incorporated by reference. Proline-rich Gla (G-carboxyglutamic acid) Polypeptide 1 (PRRG1, Accession NM_{—}000950) is another VGAM104 host target gene. PRRG1 BINDING SITE is HOST TARGET binding site found in the 3′ untranslated region of mRNA encoded by PRRG1, corresponding to a HOST TARGET binding site such as BINDING SITE I, BINDING SITE II or BINDING SITE III. Table 2 illustrates the complementarity of the nucleotide sequences of PRRG1 BINDING SITE, designated SEQ ID:789, to the nucleotide sequence of VGAM104 RNA, herein designated VGAM RNA, also designated SEQ ID:439.

Another function of VGAM104 is therefore inhibition of Proline-rich Gla (G-carboxyglutamic acid) Polypeptide 1 (PRRG1, Accession NM_{—}000950). Accordingly, utilities of VGAM104 include diagnosis, prevention and treatment of diseases and clinical conditions associated with PRRG1. LOC149320 (Accession XM_{—}047557) is another VGAM104 host target gene. LOC149320 BINDING SITE is HOST TARGET binding site found in the 3′ untranslated region of mRNA encoded by LOC149320, corresponding to a HOST TARGET binding site such as BINDING SITE I, BINDING SITE II or BINDING SITE III. Table 2 illustrates the complementarity of the nucleotide sequences of LOC149320 BINDING SITE, designated SEQ ID:2892, to the nucleotide sequence of VGAM104 RNA, herein designated VGAM RNA, also designated SEQ ID:439.

Another function of VGAM104 is therefore inhibition of LOC149320 (Accession XM_{—}047557). Accordingly, utilities of VGAM104 include diagnosis, prevention and treatment of diseases and clinical conditions associated with LOC149320. LOC163782 (Accession XM_{—}089138) is another VGAM104 host target gene. LOC163782 BINDING SITE is HOST TARGET binding site found in the 5′ untranslated region of mRNA encoded by LOC163782, corresponding to a HOST TARGET binding site such as BINDING SITE I, BINDING SITE II or BINDING SITE III. Table 2 illustrates the complementarity of the nucleotide sequences of LOC163782 BINDING SITE, designated SEQ ID:3234, to the nucleotide sequence of VGAM104 RNA, herein designated VGAM RNA, also designated SEQ ID:439.

Another function of VGAM104 is therefore inhibition of LOC163782 (Accession XM_{—}089138). Accordingly, utilities of VGAM104 include diagnosis, prevention and treatment of diseases and clinical conditions associated with LOC163782. FIG. 1 further provides a conceptual description of a novel bioinformatically detected viral gene of the present invention, referred to here as Viral Genomic Address Messenger 105 (VGAM105) viral gene, which modulates expression of respective host target genes thereof, the function and utility of which host target genes is known in the art.

VGAM105 is a novel bioinformatically detected regulatory, non protein coding, viral micro RNA (miRNA) gene. The method by which VGAM105 was detected is described hereinabove with reference to FIGS. 1-8.

VGAM105 gene, herein designated VGAM GENE, is a viral gene contained in the genome of Vaccinia Virus. VGAM105 host target gene, herein designated VGAM HOST TARGET GENE, is a human gene contained in the human genome.

VGAM105 gene encodes a VGAM105 precursor RNA, herein designated VGAM PRECURSOR RNA. Similar to other miRNA genes, and unlike most ordinary genes, VGAM105 precursor RNA does not encode a protein. A nucleotide sequence identical or highly similar to the nucleotide sequence of VGAM105 precursor RNA is designated SEQ ID:91, and is provided hereinbelow with reference to the sequence listing part. Nucleotide sequence SEQ ID:91 is located at position 44202 relative to the genome of Vaccinia Virus.

VGAM105 precursor RNA folds onto itself, forming VGAM105 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM105 folded precursor RNA into VGAM105 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM105 RNA is designated SEQ ID:440, and is provided hereinbelow with reference to the sequence listing part.

VGAM105 host target gene, herein designated VGAM HOST TARGET GENE, encodes a corresponding messenger RNA, VGAM105 host target RNA, herein designated VGAM HOST TARGET RNA. VGAM105 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM105 RNA, herein designated VGAM RNA, binds complementarily to one or more host target binding sites located in untranslated regions of VGAM105 host target RNA, herein designated VGAM HOST TARGET RNA. This complementary binding is due to the fact that the nucleotide sequence of VGAM105 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM105 RNA, herein designated VGAM RNA, may have a different number of host target binding sites in untranslated regions of a VGAM105 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM105 RNA, herein designated VGAM RNA, to host target binding sites on VGAM105 host target RNA, herein designated VGAM HOST TARGET RNA, such as BINDING SITE I, BINDING SITE II and BINDING SITE III, inhibits translation of VGAM105 host target RNA into VGAM105 host target protein, herein designated VGAM HOST TARGET PROTEIN. VGAM host target protein is therefore outlined by a broken line.

It is appreciated that VGAM105 host target gene, herein designated VGAM HOST TARGET GENE, in fact represents a plurality of VGAM105 host target genes. The mRNA of each one of this plurality of VGAM105 host target genes comprises one or more host target binding sites, each having a nucleotide sequence which is at least partly complementary to VGAM105 RNA, herein designated VGAM RNA, and which when bound by VGAM105 RNA causes inhibition of translation of respective one or more VGAM105 host target proteins.

It is further appreciated by one skilled in the art that the mode of translational inhibition illustrated by FIG. 1 with specific reference to translational inhibition exerted by VGAM105 gene, herein designated VGAM GENE, on one or more VGAM105 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM105 is inhibition of expression of host target genes, as part of a novel viral mechanism of attacking a host. Accordingly, utilities of VGAM105 include diagnosis, prevention and treatment of viral infection by Vaccinia Virus. Specific functions, and accordingly utilities, of VGAM105 correlate with, and may be deduced from, the identity of the host target genes which VGAM105 binds and inhibits, and the function of these host target genes, as elaborated hereinbelow.

Nucleotide sequences of the VGAM105 precursor RNA, herein designated VGAM PRECURSOR RNA, and of the ‘diced’ VGAM105 RNA, herein designated VGAM RNA, and a schematic representation of the secondary folding of VGAM105 folded precursor RNA, herein designated VGAM FOLDED PRECURSOR RNA, of VGAM105 are further described hereinbelow with reference to Table 1.

Nucleotide sequences of host target binding sites, such as BINDING SITE-I, BINDING SITE-II and BINDING SITE-III of FIG. 1, found on VGAM105 host target RNA, and schematic representation of the complementarity of each of these host target binding sites to VGAM105 RNA, herein designated VGAM RNA, are described hereinbelow with reference to Table 2.

As mentioned hereinabove with reference to FIG. 1, a function of VGAM105 gene, herein designated VGAM is inhibition of expression of VGAM105 target genes. It is appreciated that specific functions, and accordingly utilities, of VGAM105 correlate with, and may be deduced from, the identity of the target genes which VGAM105 binds and inhibits, and the function of these target genes, as elaborated hereinbelow.

FLJ12668 (Accession NM_{—}024997) is a VGAM105 host target gene. FLJ12668 BINDING SITE is HOST TARGET binding site found in the 3′ untranslated region of mRNA encoded by FLJ12668, corresponding to a HOST TARGET binding site such as BINDING SITE I, BINDING SITE II or BINDING SITE III. Table 2 illustrates the complementarity of the nucleotide sequences of FLJ12668 BINDING SITE, designated SEQ ID:2123, to the nucleotide sequence of VGAM105 RNA, herein designated VGAM RNA, also designated SEQ ID:440.

A function of VGAM105 is therefore inhibition of FLJ12668 (Accession NM_{—}024997). Accordingly, utilities of VGAM105 include diagnosis, prevention and treatment of diseases and clinical conditions associated with FLJ12668. KIAA0016 (Accession NM_{—}014765) is another VGAM105 host target gene. KIAA0016 BINDING SITE is HOST TARGET binding site found in the 3′ untranslated region of mRNA encoded by KIAA0016, corresponding to a HOST TARGET binding site such as BINDING SITE I, BINDING SITE II or BINDING SITE III. Table 2 illustrates the complementarity of the nucleotide sequences of KIAA0016 BINDING SITE, designated SEQ ID:1542, to the nucleotide sequence of VGAM105 RNA, herein designated VGAM RNA, also designated SEQ ID:440.

Another function of VGAM105 is therefore inhibition of KIAA0016 (Accession NM_{—}014765). Accordingly, utilities of VGAM105 include diagnosis, prevention and treatment of diseases and clinical conditions associated with KIAA0016. ZAK (Accession NM_{—}016653) is another VGAM105 host target gene. ZAK BINDING SITE is HOST TARGET binding site found in the 3′ untranslated region of mRNA encoded by ZAK, corresponding to a HOST TARGET binding site such as BINDING SITE I, BINDING SITE II or BINDING SITE III. Table 2 illustrates the complementarity of the nucleotide sequences of ZAK BINDING SITE, designated SEQ ID:1708, to the nucleotide sequence of VGAM105 RNA, herein designated VGAM RNA, also designated SEQ ID:440.

Another function of VGAM105 is therefore inhibition of ZAK (Accession NM_{—}016653). Accordingly, utilities of VGAM105 include diagnosis, prevention and treatment of diseases and clinical conditions associated with ZAK. LOC58489 (Accession XM_{—}051862) is another VGAM105 host target gene. LOC58489 BINDING SITE is HOST TARGET binding site found in the 3′ untranslated region of mRNA encoded by LOC58489, corresponding to a HOST TARGET binding site such as BINDING SITE I, BINDING SITE II or BINDING SITE III. Table 2 illustrates the complementarity of the nucleotide sequences of LOC58489 BINDING SITE, designated SEQ ID:2952, to the nucleotide sequence of VGAM105 RNA, herein designated VGAM RNA, also designated SEQ ID:440.

Another function of VGAM105 is therefore inhibition of LOC58489 (Accession XM_{—}051862). Accordingly, utilities of VGAM105 include diagnosis, prevention and treatment of diseases and clinical conditions associated with LOC58489. FIG. 1 further provides a conceptual description of a novel bioinformatically detected viral gene of the present invention, referred to here as Viral Genomic Address Messenger 106 (VGAM106) viral gene, which modulates expression of respective host target genes thereof, the function and utility of which host target genes is known in the art.

VGAM106 is a novel bioinformatically detected regulatory, non protein coding, viral micro RNA (miRNA) gene. The method by which VGAM106 was detected is described hereinabove with reference to FIGS. 1-8.

VGAM106 gene, herein designated VGAM GENE, is a viral gene contained in the genome of Vaccinia Virus. VGAM106 host target gene, herein designated VGAM HOST TARGET GENE, is a human gene contained in the human genome.

VGAM106 gene encodes a VGAM106 precursor RNA, herein designated VGAM PRECURSOR RNA. Similar to other miRNA genes, and unlike most ordinary genes, VGAM106 precursor RNA does not encode a protein. A nucleotide sequence identical or highly similar to the nucleotide sequence of VGAM106 precursor RNA is designated SEQ ID:92, and is provided hereinbelow with reference to the sequence listing part. Nucleotide sequence SEQ ID:92 is located at position 43627 relative to the genome of Vaccinia Virus.

VGAM106 precursor RNA folds onto itself, forming VGAM106 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM106 folded precursor RNA into VGAM106 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM106 RNA is designated SEQ ID:441, and is provided hereinbelow with reference to the sequence listing part.

VGAM106 host target gene, herein designated VGAM HOST TARGET GENE, encodes a corresponding messenger RNA, VGAM106 host target RNA, herein designated VGAM HOST TARGET RNA. VGAM106 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM106 RNA, herein designated VGAM RNA, binds complementarily to one or more host target binding sites located in untranslated regions of VGAM106 host target RNA, herein designated VGAM HOST TARGET RNA. This complementary binding is due to the fact that the nucleotide sequence of VGAM106 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM106 RNA, herein designated VGAM RNA, may have a different number of host target binding sites in untranslated regions of a VGAM106 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM106 RNA, herein designated VGAM RNA, to host target binding sites on VGAM106 host target RNA, herein designated VGAM HOST TARGET RNA, such as BINDING SITE I, BINDING SITE II and BINDING SITE III, inhibits translation of VGAM106 host target RNA into VGAM106 host target protein, herein designated VGAM HOST TARGET PROTEIN. VGAM host target protein is therefore outlined by a broken line.

It is appreciated that VGAM106 host target gene, herein designated VGAM HOST TARGET GENE, in fact represents a plurality of VGAM106 host target genes. The mRNA of each one of this plurality of VGAM106 host target genes comprises one or more host target binding sites, each having a nucleotide sequence which is at least partly complementary to VGAM106 RNA, herein designated VGAM RNA, and which when bound by VGAM106 RNA causes inhibition of translation of respective one or more VGAM106 host target proteins.

It is further appreciated by one skilled in the art that the mode of translational inhibition illustrated by FIG. 1 with specific reference to translational inhibition exerted by VGAM106 gene, herein designated VGAM GENE, on one or more VGAM106 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM106 is inhibition of expression of host target genes, as part of a novel viral mechanism of attacking a host. Accordingly, utilities of VGAM106 include diagnosis, prevention and treatment of viral infection by Vaccinia Virus. Specific functions, and accordingly utilities, of VGAM106 correlate with, and may be deduced from, the identity of the host target genes which VGAM106 binds and inhibits, and the function of these host target genes, as elaborated hereinbelow.

Nucleotide sequences of the VGAM106 precursor RNA, herein designated VGAM PRECURSOR RNA, and of the ‘diced’ VGAM106 RNA, herein designated VGAM RNA, and a schematic representation of the secondary folding of VGAM106 folded precursor RNA, herein designated VGAM FOLDED PRECURSOR RNA, of VGAM106 are further described hereinbelow with reference to Table 1.

Nucleotide sequences of host target binding sites, such as BINDING SITE-I, BINDING SITE-II and BINDING SITE-III of FIG. 1, found on VGAM106 host target RNA, and schematic representation of the complementarity of each of these host target binding sites to VGAM106 RNA, herein designated VGAM RNA, are described hereinbelow with reference to Table 2.

As mentioned hereinabove with reference to FIG. 1, a function of VGAM106 gene, herein designated VGAM is inhibition of expression of VGAM106 target genes. It is appreciated that specific functions, and accordingly utilities, of VGAM106 correlate with, and may be deduced from, the identity of the target genes which VGAM106 binds and inhibits, and the function of these target genes, as elaborated hereinbelow.

Interleukin 1 Receptor, Type I (IL1R1, Accession NM_{—}000877) is a VGAM106 host target gene. IL1R1 BINDING SITE is HOST TARGET binding site found in the 3′ untranslated region of mRNA encoded by IL1R1, corresponding to a HOST TARGET binding site such as BINDING SITE I, BINDING SITE II or BINDING SITE III. Table 2 illustrates the complementarity of the nucleotide sequences of IL1R1 BINDING SITE, designated SEQ ID:782, to the nucleotide sequence of VGAM106 RNA, herein designated VGAM RNA, also designated SEQ ID:441.

A function of VGAM106 is therefore inhibition of Interleukin 1 Receptor, Type I (IL1R1, Accession NM_{—}000877), a gene which is a receptor for interleukin-1 alpha (il-1a), beta (il-1b), and interleukin-1 receptor antagonist protein (il-1ra). Accordingly, utilities of VGAM106 include diagnosis, prevention and treatment of diseases and clinical conditions associated with IL1R1. The function of IL1R1 has been established by previous studies. Interleukin-1, which has a role as a mediator in inflammation, actually consists of 2 separate but related proteins, IL1-alpha (OMIM Ref. No. 147720) and IL1-beta (OMIM Ref. No. 147760). Dower et al. (1986) showed that the cell surface receptors for the 2 forms of interleukin-1 are identical. Sims et al. (1989) cloned the human ILR gene and compared it with the mouse gene. Both contain a single membrane-spanning segment, a large cytoplasmic region, and an extracellular, IL1-binding portion composed of 3 immunoglobulin-like domains. The ILR gene expressed in human dermal fibroblasts was found to be identical to that expressed in T cells. By a combination of somatic cell hybrid analysis and chromosomal in situ hybridization, Copeland et al. (1991) mapped the ILR gene to human chromosome 2q12. By RFLP analysis in interspecific backcrosses, Copeland et al. (1991) mapped the corresponding mouse gene at the centromeric end of chromosome 1, a region homologous to a portion of human chromosome 2

Full details of the abovementioned studies are described in the following publications, the disclosure of which are hereby incorporated by reference:
Dower, S. K.; Kronheim, S. R.; Hopp, T. P.; Cantrell, M.; Deeley, M.; Gillis, S.; Henney, C. S.; Urdal, D. L.: The cell surface receptors for interleukin-1(alpha) and interleukin-1(beta) are identical. Nature 324: 266-268, 1986.; and
Sims, J. E.; Acres, R. B.; Grubin, C. E.; McMahan, C. J.; Wignall, J. M.; March, C. J.; Dower, S. K.: Cloning the interleukin 1 receptor from human T cells. Proc. Nat. Acad. Sci. 86: 89.

Further studies establishing the function and utilities of IL1R1 are found in John Hopkins OMIM database record ID 147810, and in sited publications numbered 691-694 listed in the bibliography section hereinbelow, which are also hereby incorporated by reference. Suppressor of Fused Homolog (Drosophila) (SUFU, Accession NM_{—}016169) is another VGAM106 host target gene. SUFU BINDING SITE is HOST TARGET binding site found in the 3′ untranslated region of mRNA encoded by SUFU, corresponding to a HOST TARGET binding site such as BINDING SITE I, BINDING SITE II or BINDING SITE III. Table 2 illustrates the complementarity of the nucleotide sequences of SUFU BINDING SITE, designated SEQ ID: 1668, to the nucleotide sequence of VGAM106 RNA, herein designated VGAM RNA, also designated SEQ ID:441.

Another function of VGAM106 is therefore inhibition of Suppressor of Fused Homolog (Drosophila) (SUFU, Accession NM_{—}016169). Accordingly, utilities of VGAM106 include diagnosis, prevention and treatment of diseases and clinical conditions associated with SUFU. CGRP-RCP (Accession NM_{—}014478) is another VGAM106 host target gene. CGRP-RCP BINDING SITE is HOST TARGET binding site found in the 5′ untranslated region of mRNA encoded by CGRP-RCP, corresponding to a HOST TARGET binding site such as BINDING SITE I, BINDING SITE II or BINDING SITE III. Table 2 illustrates the complementarity of the nucleotide sequences of CGRP-RCP BINDING SITE, designated SEQ ID:1503, to the nucleotide sequence of VGAM106 RNA, herein designated VGAM RNA, also designated SEQ ID:441.

Another function of VGAM106 is therefore inhibition of CGRP-RCP (Accession NM_{—}014478). Accordingly, utilities of VGAM106 include diagnosis, prevention and treatment of diseases and clinical conditions associated with CGRP-RCP. LOC152348 (Accession XM_{—}098204) is another VGAM106 host target gene. LOC152348 BINDING SITE is HOST TARGET binding site found in the 5′ untranslated region of mRNA encoded by LOC152348, corresponding to a HOST TARGET binding site such as BINDING SITE I, BINDING SITE II or BINDING SITE III. Table 2 illustrates the complementarity of the nucleotide sequences of LOC152348 BINDING SITE, designated SEQ ID:3358, to the nucleotide sequence of VGAM106 RNA, herein designated VGAM RNA, also designated SEQ ID:441.

Another function of VGAM106 is therefore inhibition of LOC152348 (Accession XM_{—}098204). Accordingly, utilities of VGAM106 include diagnosis, prevention and treatment of diseases and clinical conditions associated with LOC152348. LOC158219 (Accession XM_{—}088514) is another VGAM106 host target gene. LOC158219 BINDING SITE is HOST TARGET binding site found in the 3′ untranslated region of mRNA encoded by LOC158219, corresponding to a HOST TARGET binding site such as BINDING SITE I, BINDING SITE II or BINDING SITE III. Table 2 illustrates the complementarity of the nucleotide sequences of LOC158219 BINDING SITE, designated SEQ ID:3214, to the nucleotide sequence of VGAM106 RNA, herein designated VGAM RNA, also designated SEQ ID:441.

Another function of VGAM106 is therefore inhibition of LOC158219 (Accession XM_{—}088514). Accordingly, utilities of VGAM106 include diagnosis, prevention and treatment of diseases and clinical conditions associated with LOC158219. LOC255177 (Accession XM_{—}172941) is another VGAM106 host target gene. LOC255177 BINDING SITE is HOST TARGET binding site found in the 3′ untranslated region of mRNA encoded by LOC255177, corresponding to a HOST TARGET binding site such as BINDING SITE I, BINDING SITE II or BINDING SITE III. Table 2 illustrates the complementarity of the nucleotide sequences of LOC255177 BINDING SITE, designated SEQ ID:3711, to the nucleotide sequence of VGAM106 RNA, herein designated VGAM RNA, also designated SEQ ID:441.

Another function of VGAM106 is therefore inhibition of LOC255177 (Accession XM_{—}172941). Accordingly, utilities of VGAM106 include diagnosis, prevention and treatment of diseases and clinical conditions associated with LOC255177. LOC90522 (Accession XM_{—}032285) is another VGAM106 host target gene. LOC90522 BINDING SITE is HOST TARGET binding site found in the 3′ untranslated region of mRNA encoded by LOC90522, corresponding to a HOST TARGET binding site such as BINDING SITE I, BINDING SITE II or BINDING SITE III. Table 2 illustrates the complementarity of the nucleotide sequences of LOC90522 BINDING SITE, designated SEQ ID:2651, to the nucleotide sequence of VGAM106 RNA, herein designated VGAM RNA, also designated SEQ ID:441.

Another function of VGAM106 is therefore inhibition of LOC90522 (Accession XM_{—}032285). Accordingly, utilities of VGAM106 include diagnosis, prevention and treatment of diseases and clinical conditions associated with LOC90522. FIG. 1 further provides a conceptual description of a novel bioinformatically detected viral gene of the present invention, referred to here as Viral Genomic Address Messenger 107 (VGAM107) viral gene, which modulates expression of respective host target genes thereof, the function and utility of which host target genes is known in the art.

VGAM107 is a novel bioinformatically detected regulatory, non protein coding, viral micro RNA (miRNA) gene. The method by which VGAM107 was detected is described hereinabove with reference to FIGS. 1-8.

VGAM107 gene, herein designated VGAM GENE, is a viral gene contained in the genome of Vaccinia Virus. VGAM107 host target gene, herein designated VGAM HOST TARGET GENE, is a human gene contained in the human genome.

VGAM107 gene encodes a VGAM107 precursor RNA, herein designated VGAM PRECURSOR RNA. Similar to other miRNA genes, and unlike most ordinary genes, VGAM107 precursor RNA does not encode a protein. A nucleotide sequence identical or highly similar to the nucleotide sequence of VGAM107 precursor RNA is designated SEQ ID:93, and is provided hereinbelow with reference to the sequence listing part. Nucleotide sequence SEQ ID:93 is located at position 44848 relative to the genome of Vaccinia Virus.

VGAM107 precursor RNA folds onto itself, forming VGAM107 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM107 folded precursor RNA into VGAM107 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM107 RNA is designated SEQ ID:442, and is provided hereinbelow with reference to the sequence listing part.

VGAM107 host target gene, herein designated VGAM HOST TARGET GENE, encodes a corresponding messenger RNA, VGAM107 host target RNA, herein designated VGAM HOST TARGET RNA. VGAM107 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM107 RNA, herein designated VGAM RNA, binds complementarily to one or more host target binding sites located in untranslated regions of VGAM107 host target RNA, herein designated VGAM HOST TARGET RNA. This complementary binding is due to the fact that the nucleotide sequence of VGAM107 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM107 RNA, herein designated VGAM RNA, may have a different number of host target binding sites in untranslated regions of a VGAM107 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM107 RNA, herein designated VGAM RNA, to host target binding sites on VGAM107 host target RNA, herein designated VGAM HOST TARGET RNA, such as BINDING SITE I, BINDING SITE II and BINDING SITE III, inhibits translation of VGAM107 host target RNA into VGAM107 host target protein, herein designated VGAM HOST TARGET PROTEIN. VGAM host target protein is therefore outlined by a broken line.

It is appreciated that VGAM107 host target gene, herein designated VGAM HOST TARGET GENE, in fact represents a plurality of VGAM107 host target genes. The mRNA of each one of this plurality of VGAM107 host target genes comprises one or more host target binding sites, each having a nucleotide sequence which is at least partly complementary to VGAM107 RNA, herein designated VGAM RNA, and which when bound by VGAM107 RNA causes inhibition of translation of respective one or more VGAM107 host target proteins.

It is further appreciated by one skilled in the art that the mode of translational inhibition illustrated by FIG. 1 with specific reference to translational inhibition exerted by VGAM107 gene, herein designated VGAM GENE, on one or more VGAM107 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM107 is inhibition of expression of host target genes, as part of a novel viral mechanism of attacking a host. Accordingly, utilities of VGAM107 include diagnosis, prevention and treatment of viral infection by Vaccinia Virus. Specific functions, and accordingly utilities, of VGAM107 correlate with, and may be deduced from, the identity of the host target genes which VGAM107 binds and inhibits, and the function of these host target genes, as elaborated hereinbelow.

Nucleotide sequences of the VGAM107 precursor RNA, herein designated VGAM PRECURSOR RNA, and of the ‘diced’ VGAM107 RNA, herein designated VGAM RNA, and a schematic representation of the secondary folding of VGAM107 folded precursor RNA, herein designated VGAM FOLDED PRECURSOR RNA, of VGAM107 are further described hereinbelow with reference to Table 1.

Nucleotide sequences of host target binding sites, such as BINDING SITE-I, BINDING SITE-II and BINDING SITE-III of FIG. 1, found on VGAM107 host target RNA, and schematic representation of the complementarity of each of these host target binding sites to VGAM107 RNA, herein designated VGAM RNA, are described hereinbelow with reference to Table 2.

As mentioned hereinabove with reference to FIG. 1, a function of VGAM107 gene, herein designated VGAM is inhibition of expression of VGAM107 target genes. It is appreciated that specific functions, and accordingly utilities, of VGAM107 correlate with, and may be deduced from, the identity of the target genes which VGAM107 binds and inhibits, and the function of these target genes, as elaborated hereinbelow.

Carcinoembryonic Antigen-related Cell Adhesion Molecule 4 (CEACAM4, Accession NM_{—}001817) is a VGAM107 host target gene. CEACAM4 BINDING SITE is HOST TARGET binding site found in the 3′ untranslated region of mRNA encoded by CEACAM4, corresponding to a HOST TARGET binding site such as BINDING SITE I, BINDING SITE II or BINDING SITE III. Table 2 illustrates the complementarity of the nucleotide sequences of CEACAM4 BINDING SITE, designated SEQ ID:860, to the nucleotide sequence of VGAM107 RNA, herein designated VGAM RNA, also designated SEQ ID:442.

A function of VGAM107 is therefore inhibition of Carcinoembryonic Antigen-related Cell Adhesion Molecule 4 (CEACAM4, Accession NM_{—}001817). Accordingly, utilities of VGAM107 include diagnosis, prevention and treatment of diseases and clinical conditions associated with CEACAM4. MGC11115 (Accession NM_{—}032310) is another VGAM107 host target gene. MGC11115 BINDING SITE is HOST TARGET binding site found in the 3′ untranslated region of mRNA encoded by MGC11115, corresponding to a HOST TARGET binding site such as BINDING SITE I, BINDING SITE II or BINDING SITE III. Table 2 illustrates the complementarity of the nucleotide sequences of MGC11115 BINDING SITE, designated SEQ ID:2244, to the nucleotide sequence of VGAM107 RNA, herein designated VGAM RNA, also designated SEQ ID:442.

Another function of VGAM107 is therefore inhibition of MGC11115 (Accession NM_{—}032310). Accordingly, utilities of VGAM107 include diagnosis, prevention and treatment of diseases and clinical conditions associated with MGC11115. PRO2714 (Accession NM_{—}018534) is another VGAM107 host target gene. PRO2714 BINDING SITE is HOST TARGET binding site found in the 5′ untranslated region of mRNA encoded by PRO2714, corresponding to a HOST TARGET binding site such as BINDING SITE I, BINDING SITE II or BINDING SITE III. Table 2 illustrates the complementarity of the nucleotide sequences of PRO2714 BINDING SITE, designated SEQ ID:1837, to the nucleotide sequence of VGAM107 RNA, herein designated VGAM RNA, also designated SEQ ID:442.

Another function of VGAM107 is therefore inhibition of PRO2714 (Accession NM_{—}018534). Accordingly, utilities of VGAM107 include diagnosis, prevention and treatment of diseases and clinical conditions associated with PRO2714. LOC147057 (Accession XM_{—}097166) is another VGAM107 host target gene. LOC147057 BINDING SITE is HOST TARGET binding site found in the 3′ untranslated region of mRNA encoded by LOC147057, corresponding to a HOST TARGET binding site such as BINDING SITE I, BINDING SITE II or BINDING SITE III. Table 2 illustrates the complementarity of the nucleotide sequences of LOC147057 BINDING SITE, designated SEQ ID:3288, to the nucleotide sequence of VGAM107 RNA, herein designated VGAM RNA, also designated SEQ ID:442.

Another function of VGAM107 is therefore inhibition of LOC147057 (Accession XM_{—}097166). Accordingly, utilities of VGAM107 include diagnosis, prevention and treatment of diseases and clinical conditions associated with LOC147057. LOC147976 (Accession XM_{—}085980) is another VGAM107 host target gene. LOC147976 BINDING SITE is HOST TARGET binding site found in the 3′ untranslated region of mRNA encoded by LOC147976, corresponding to a HOST TARGET binding site such as BINDING SITE I, BINDING SITE II or BINDING SITE III. Table 2 illustrates the complementarity of the nucleotide sequences of LOC147976 BINDING SITE, designated SEQ ID:3114, to the nucleotide sequence of VGAM107 RNA, herein designated VGAM RNA, also designated SEQ ID:442.

Another function of VGAM107 is therefore inhibition of LOC147976 (Accession XM_{—}085980). Accordingly, utilities of VGAM107 include diagnosis, prevention and treatment of diseases and clinical conditions associated with LOC147976. LOC57100 (Accession NM_{—}020372) is another VGAM107 host target gene. LOC57100 BINDING SITE is HOST TARGET binding site found in the 3′ untranslated region of mRNA encoded by LOC57100, corresponding to a HOST TARGET binding site such as BINDING SITE I, BINDING SITE II or BINDING SITE III. Table 2 illustrates the complementarity of the nucleotide sequences of LOC57100 BINDING SITE, designated SEQ ID:1917, to the nucleotide sequence of VGAM107 RNA, herein designated VGAM RNA, also designated SEQ ID:442.

Another function of VGAM107 is therefore inhibition of LOC57100 (Accession NM_{—}020372). Accordingly, utilities of VGAM107 include diagnosis, prevention and treatment of diseases and clinical conditions associated with LOC57100. FIG. 1 further provides a conceptual description of a novel bioinformatically detected viral gene of the present invention, referred to here as Viral Genomic Address Messenger 108 (VGAM108) viral gene, which modulates expression of respective host target genes thereof, the function and utility of which host target genes is known in the art.

VGAM108 is a novel bioinformatically detected regulatory, non protein coding, viral micro RNA (miRNA) gene. The method by which VGAM108 was detected is described hereinabove with reference to FIGS. 1-8.

VGAM108 gene, herein designated VGAM GENE, is a viral gene contained in the genome of Vaccinia Virus. VGAM108 host target gene, herein designated VGAM HOST TARGET GENE, is a human gene contained in the human genome.

VGAM108 gene encodes a VGAM108 precursor RNA, herein designated VGAM PRECURSOR RNA. Similar to other miRNA genes, and unlike most ordinary genes, VGAM108 precursor RNA does not encode a protein. A nucleotide sequence identical or highly similar to the nucleotide sequence of VGAM108 precursor RNA is designated SEQ ID:94, and is provided hereinbelow with reference to the sequence listing part. Nucleotide sequence SEQ ID:94 is located at position 43844 relative to the genome of Vaccinia Virus.

VGAM108 precursor RNA folds onto itself, forming VGAM108 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM108 folded precursor RNA into VGAM108 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM108 RNA is designated SEQ ID:443, and is provided hereinbelow with reference to the sequence listing part.

VGAM108 host target gene, herein designated VGAM HOST TARGET GENE, encodes a corresponding messenger RNA, VGAM108 host target RNA, herein designated VGAM HOST TARGET RNA. VGAM108 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM108 RNA, herein designated VGAM RNA, binds complementarily to one or more host target binding sites located in untranslated regions of VGAM108 host target RNA, herein designated VGAM HOST TARGET RNA. This complementary binding is due to the fact that the nucleotide sequence of VGAM108 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM108 RNA, herein designated VGAM RNA, may have a different number of host target binding sites in untranslated regions of a VGAM108 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM108 RNA, herein designated VGAM RNA, to host target binding sites on VGAM108 host target RNA, herein designated VGAM HOST TARGET RNA, such as BINDING SITE I, BINDING SITE II and BINDING SITE III, inhibits translation of VGAM108 host target RNA into VGAM108 host target protein, herein designated VGAM HOST TARGET PROTEIN. VGAM host target protein is therefore outlined by a broken line.

It is appreciated that VGAM108 host target gene, herein designated VGAM HOST TARGET GENE, in fact represents a plurality of VGAM108 host target genes. The mRNA of each one of this plurality of VGAM108 host target genes comprises one or more host target binding sites, each having a nucleotide sequence which is at least partly complementary to VGAM108 RNA, herein designated VGAM RNA, and which when bound by VGAM108 RNA causes inhibition of translation of respective one or more VGAM108 host target proteins.

It is further appreciated by one skilled in the art that the mode of translational inhibition illustrated by FIG. 1 with specific reference to translational inhibition exerted by VGAM108 gene, herein designated VGAM GENE, on one or more VGAM108 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM108 is inhibition of expression of host target genes, as part of a novel viral mechanism of attacking a host. Accordingly, utilities of VGAM108 include diagnosis, prevention and treatment of viral infection by Vaccinia Virus. Specific functions, and accordingly utilities, of VGAM108 correlate with, and may be deduced from, the identity of the host target genes which VGAM108 binds and inhibits, and the function of these host target genes, as elaborated hereinbelow.

Nucleotide sequences of the VGAM108 precursor RNA, herein designated VGAM PRECURSOR RNA, and of the ‘diced’ VGAM108 RNA, herein designated VGAM RNA, and a schematic representation of the secondary folding of VGAM108 folded precursor RNA, herein designated VGAM FOLDED PRECURSOR RNA, of VGAM108 are further described hereinbelow with reference to Table 1.

Nucleotide sequences of host target binding sites, such as BINDING SITE-I, BINDING SITE-II and BINDING SITE-III of FIG. 1, found on VGAM108 host target RNA, and schematic representation of the complementarity of each of these host target binding sites to VGAM108 RNA, herein designated VGAM RNA, are described hereinbelow with reference to Table 2.

As mentioned hereinabove with reference to FIG. 1, a function of VGAM108 gene, herein designated VGAM is inhibition of expression of VGAM108 target genes. It is appreciated that specific functions, and accordingly utilities, of VGAM108 correlate with, and may be deduced from, the identity of the target genes which VGAM108 binds and inhibits, and the function of these target genes, as elaborated hereinbelow.

E2F Transcription Factor 3 (E2F3, Accession NM_{—}001949) is a VGAM108 host target gene. E2F3 BINDING SITE is HOST TARGET binding site found in the 3′ untranslated region of mRNA encoded by E2F3, corresponding to a HOST TARGET binding site such as BINDING SITE I, BINDING SITE II or BINDING SITE III. Table 2 illustrates the complementarity of the nucleotide sequences of E2F3 BINDING SITE, designated SEQ ID:875, to the nucleotide sequence of VGAM108 RNA, herein designated VGAM RNA, also designated SEQ ID:443.

A function of VGAM108 is therefore inhibition of E2F Transcription Factor 3 (E2F3, Accession NM_{—}001949), a gene which binds dna and controls cell-cycle progression from g1 to s phase. Accordingly, utilities of VGAM108 include diagnosis, prevention and treatment of diseases and clinical conditions associated with E2F3. The function of E2F3 has been established by previous studies. MYC (OMIM Ref. No. 190080) induces transcription of the E2F1, E2F2 (OMIM Ref. No. 600426), and E2F3 genes. Using primary mouse embryo fibroblasts deleted for individual E2f genes, Leone et al. (2001) showed that MYC-induced S phase and apoptosis requires distinct E2F activities. The ability of Myc to induce S phase was impaired in the absence of either E2f2 or E2f3 but not E2f1 or E2f4 (OMIM Ref. No. 600659). In contrast, the ability of Myc to induce apoptosis was markedly reduced in cells deleted for E2fl but not E2f2 or E2f3. The authors proposed that the induction of specific E2F activities is an essential component in the MYC pathways that control cell proliferation and cell fate decisions. Animal model experiments lend further support to the function of E2F3. Cloud et al. (2002) generated E2f3-null mice. They found that E2f3 was essential for embryonic viability in the pure 129/Sv background, but that the presence of C57BL/6 alleles yielded some adult survivors. Although growth retarded, surviving E2f3 −/− animals were initially healthy and exhibited no obvious tumor phenotype. They died prematurely, however, with signs typical of congestive heart failure, a defect completely distinct from those reported in E2f1-null mice. Cloud et al. (2002) also generated E2f1/E2f3 compound mutant mice and found that almost all of the developmental and age-related defects arising in the individual E2f1- or E2f3-null mice were exacerbated by the mutation of the other E2f. One major difference in the properties of E2f1 and E2f3 loss was that, either alone or in combination with loss of E2f1, E2f3 mutants did not show an increase in the incidence of tumor formation.

It is appreciated that the abovementioned animal model for E2F3 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Leone, G.; Sears, R.; Huang, E.; Rempel, R.; Nuckolls, F.; Park, C.-H.; Giangrande, P.; Wu, L.; Saavedra, H. I.; Field, S. J.; Thompson, M. A.; Yang, H.; Fujiwara, Y.; Greenberg, M. E.; Orkin, S.; Smith, C.; Nevins, J. R.: Myc requires distinct E2F activities to induce S phase and apoptosis. Molec. Cell 8: 105-113, 2001.; and
Cloud, J. E.; Rogers, C.; Reza, T. L.; Ziebold, U.; Stone, J. R.; Picard, M. H.; Caron, A. M.; Bronson, R. T.; Lees, J. A.: Mutant mouse models reveal the relative roles of E2F1 and E2.

Further studies establishing the function and utilities of E2F3 are found in John Hopkins OMIM database record ID 600427, and in sited publications numbered 1714-1715, 171 and 2170 listed in the bibliography section hereinbelow, which are also hereby incorporated by reference. Sex Comb On Midleg-like 1 (Drosophila) (SCML1, Accession NM_{—}006746) is another VGAM108 host target gene. SCML1 BINDING SITE is HOST TARGET binding site found in the 3′ untranslated region of mRNA encoded by SCML1, corresponding to a HOST TARGET binding site such as BINDING SITE I, BINDING SITE II or BINDING SITE III. Table 2 illustrates the complementarity of the nucleotide sequences of SCML1 BINDING SITE, designated SEQ ID:1328, to the nucleotide sequence of VGAM108 RNA, herein designated VGAM RNA, also designated SEQ ID:443.

Another function of VGAM108 is therefore inhibition of Sex Comb On Midleg-like 1 (Drosophila) (SCML1, Accession NM_{—}006746). Accordingly, utilities of VGAM108 include diagnosis, prevention and treatment of diseases and clinical conditions associated with SCML1. KIAA1468 (Accession XM_{—}166289) is another VGAM108 host target gene. KIAA1468 BINDING SITE is HOST TARGET binding site found in the 3′ untranslated region of mRNA encoded by KIAA1468, corresponding to a HOST TARGET binding site such as BINDING SITE I, BINDING SITE II or BINDING SITE III. Table 2 illustrates the complementarity of the nucleotide sequences of KIAA1468 BINDING SITE, designated SEQ ID:3541, to the nucleotide sequence of VGAM108 RNA, herein designated VGAM RNA, also designated SEQ ID:443.

Another function of VGAM108 is therefore inhibition of KIAA1468 (Accession XM_{—}166289). Accordingly, utilities of VGAM108 include diagnosis, prevention and treatment of diseases and clinical conditions associated with KIAA1468. MGC16025 (Accession NM_{—}032923) is another VGAM108 host target gene. MGC16025 BINDING SITE is HOST TARGET binding site found in the 3′ untranslated region of mRNA encoded by MGC16025, corresponding to a HOST TARGET binding site such as BINDING SITE I, BINDING SITE II or BINDING SITE III. Table 2 illustrates the complementarity of the nucleotide sequences of MGC16025 BINDING SITE, designated SEQ ID:2295, to the nucleotide sequence of VGAM108 RNA, herein designated VGAM RNA, also designated SEQ ID:443.

Another function of VGAM108 is therefore inhibition of MGC16025 (Accession NM_{—}032923). Accordingly, utilities of VGAM108 include diagnosis, prevention and treatment of diseases and clinical conditions associated with MGC16025. LOC148545 (Accession XM_{—}086226) is another VGAM108 host target gene. LOC148545 BINDING SITE is HOST TARGET binding site found in the 3′ untranslated region of mRNA encoded by LOC148545, corresponding to a HOST TARGET binding site such as BINDING SITE I, BINDING SITE II or BINDING SITE III. Table 2 illustrates the complementarity of the nucleotide sequences of LOC148545 BINDING SITE, designated SEQ ID:3119, to the nucleotide sequence of VGAM108 RNA, herein designated VGAM RNA, also designated SEQ ID:443.

Another function of VGAM108 is therefore inhibition of LOC148545 (Accession XM_{—}086226). Accordingly, utilities of VGAM108 include diagnosis, prevention and treatment of diseases and clinical conditions associated with LOC148545. LOC163669 (Accession XM_{—}089054) is another VGAM108 host target gene. LOC163669 BINDING SITE is HOST TARGET binding site found in the 5′ untranslated region of mRNA encoded by LOC163669, corresponding to a HOST TARGET binding site such as BINDING SITE I, BINDING SITE II or BINDING SITE III. Table 2 illustrates the complementarity of the nucleotide sequences of LOC163669 BINDING SITE, designated SEQ ID:3233, to the nucleotide sequence of VGAM108 RNA, herein designated VGAM RNA, also designated SEQ ID:443.

Another function of VGAM108 is therefore inhibition of LOC163669 (Accession XM_{—}089054). Accordingly, utilities of VGAM108 include diagnosis, prevention and treatment of diseases and clinical conditions associated with LOC163669. LOC51652 (Accession NM_{—}016079) is another VGAM108 host target gene. LOC51652 BINDING SITE is HOST TARGET binding site found in the 3′ untranslated region of mRNA encoded by LOC51652, corresponding to a HOST TARGET binding site such as BINDING SITE I, BINDING SITE II or BINDING SITE III. Table 2 illustrates the complementarity of the nucleotide sequences of LOC51652 BINDING SITE, designated SEQ ID: 1660, to the nucleotide sequence of VGAM108 RNA, herein designated VGAM RNA, also designated SEQ ID:443.

Another function of VGAM108 is therefore inhibition of LOC51652 (Accession NM_{—}016079). Accordingly, utilities of VGAM108 include diagnosis, prevention and treatment of diseases and clinical conditions associated with LOC51652. FIG. 1 further provides a conceptual description of a novel bioinformatically detected viral gene of the present invention, referred to here as Viral Genomic Address Messenger 109 (VGAM109) viral gene, which modulates expression of respective host target genes thereof, the function and utility of which host target genes is known in the art.

VGAM109 is a novel bioinformatically detected regulatory, non protein coding, viral micro RNA (miRNA) gene. The method by which VGAM109 was detected is described hereinabove with reference to FIGS. 1-8.

VGAM109 gene, herein designated VGAM GENE, is a viral gene contained in the genome of Vaccinia Virus. VGAM109 host target gene, herein designated VGAM HOST TARGET GENE, is a human gene contained in the human genome.

VGAM109 gene encodes a VGAM109 precursor RNA, herein designated VGAM PRECURSOR RNA. Similar to other miRNA genes, and unlike most ordinary genes, VGAM109 precursor RNA does not encode a protein. A nucleotide sequence identical or highly similar to the nucleotide sequence of VGAM109 precursor RNA is designated SEQ ID:95, and is provided hereinbelow with reference to the sequence listing part. Nucleotide sequence SEQ ID:95 is located at position 46236 relative to the genome of Vaccinia Virus.

VGAM109 precursor RNA folds onto itself, forming VGAM109 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM109 folded precursor RNA into VGAM109 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM109 RNA is designated SEQ ID:444, and is provided hereinbelow with reference to the sequence listing part.

VGAM109 host target gene, herein designated VGAM HOST TARGET GENE, encodes a corresponding messenger RNA, VGAM109 host target RNA, herein designated VGAM HOST TARGET RNA. VGAM109 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM109 RNA, herein designated VGAM RNA, binds complementarily to one or more host target binding sites located in untranslated regions of VGAM109 host target RNA, herein designated VGAM HOST TARGET RNA. This complementary binding is due to the fact that the nucleotide sequence of VGAM109 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM109 RNA, herein designated VGAM RNA, may have a different number of host target binding sites in untranslated regions of a VGAM109 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM109 RNA, herein designated VGAM RNA, to host target binding sites on VGAM109 host target RNA, herein designated VGAM HOST TARGET RNA, such as BINDING SITE I, BINDING SITE II and BINDING SITE III, inhibits translation of VGAM109 host target RNA into VGAM109 host target protein, herein designated VGAM HOST TARGET PROTEIN. VGAM host target protein is therefore outlined by a broken line.

It is appreciated that VGAM109 host target gene, herein designated VGAM HOST TARGET GENE, in fact represents a plurality of VGAM109 host target genes. The mRNA of each one of this plurality of VGAM109 host target genes comprises one or more host target binding sites, each having a nucleotide sequence which is at least partly complementary to VGAM109 RNA, herein designated VGAM RNA, and which when bound by VGAM109 RNA causes inhibition of translation of respective one or more VGAM109 host target proteins.

It is further appreciated by one skilled in the art that the mode of translational inhibition illustrated by FIG. 1 with specific reference to translational inhibition exerted by VGAM109 gene, herein designated VGAM GENE, on one or more VGAM109 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM109 is inhibition of expression of host target genes, as part of a novel viral mechanism of attacking a host. Accordingly, utilities of VGAM109 include diagnosis, prevention and treatment of viral infection by Vaccinia Virus. Specific functions, and accordingly utilities, of VGAM109 correlate with, and may be deduced from, the identity of the host target genes which VGAM109 binds and inhibits, and the function of these host target genes, as elaborated hereinbelow.

Nucleotide sequences of the VGAM109 precursor RNA, herein designated VGAM PRECURSOR RNA, and of the ‘diced’ VGAM109 RNA, herein designated VGAM RNA, and a schematic representation of the secondary folding of VGAM109 folded precursor RNA, herein designated VGAM FOLDED PRECURSOR RNA, of VGAM109 are further described hereinbelow with reference to Table 1.

Nucleotide sequences of host target binding sites, such as BINDING SITE-I, BINDING SITE-II and BINDING SITE-III of FIG. 1, found on VGAM109 host target RNA, and schematic representation of the complementarity of each of these host target binding sites to VGAM109 RNA, herein designated VGAM RNA, are described hereinbelow with reference to Table 2.

As mentioned hereinabove with reference to FIG. 1, a function of VGAM109 gene, herein designated VGAM is inhibition of expression of VGAM109 target genes. It is appreciated that specific functions, and accordingly utilities, of VGAM109 correlate with, and may be deduced from, the identity of the target genes which VGAM109 binds and inhibits, and the function of these target genes, as elaborated hereinbelow.

Chemokine (C-X-C motif) Ligand 13 (B-cell chemoattractant) (CXCL13, Accession NM_{—}006419) is a VGAM109 host target gene. CXCL13 BINDING SITE is HOST TARGET binding site found in the 3′ untranslated region of mRNA encoded by CXCL13, corresponding to a HOST TARGET binding site such as BINDING SITE I, BINDING SITE II or BINDING SITE III. Table 2 illustrates the complementarity of the nucleotide sequences of CXCL13 BINDING SITE, designated SEQ ID:1299, to the nucleotide sequence of VGAM109 RNA, herein designated VGAM RNA, also designated SEQ ID:444.

A function of VGAM109 is therefore inhibition of Chemokine (C-X-C motif) Ligand 13 (B-cell chemoattractant) (CXCL13, Accession NM_{—}006419), a gene which plays a role in directing the migration of b lymphocytes to follicles. Accordingly, utilities of VGAM109 include diagnosis, prevention and treatment of diseases and clinical conditions associated with CXCL13. The function of CXCL13 has been established by previous studies. Burkitt lymphoma receptor-1 (BLR1; 601613), also called CXCR5, is highly expressed on Burkitt lymphoma cells and B lymphocytes. To identify the ligand of BLR1, Legler et al. (1998) searched an EST database for CXC chemokine motifs and, by PCR analysis using spleen cDNA, cloned a cDNA encoding BCA1 (B cell-attracting chemokine-1). Sequence analysis predicted that the 109-amino acid BCA1 protein contains a 22-amino acid signal sequence and an arginine residue immediately preceding the first of 4 cysteine residues, a feature typical of CXC chemokines. Northern and RNA dot blot analysis detected constitutive expression of a 1.4-kb BCA1 transcript, with highest expression in liver, followed by spleen, lymph nodes, appendix, and stomach; lower expression was detected in salivary and mammary glands, and no expression was detected in any other tissues. B lymphocytes recirculate between B cell-rich compartments (follicles or B zones) in secondary lymphoid organs, surveying for antigen. After antigen binding, B cells move to the boundary of B and T zones to interact with T-helper cells. Reif et al. (2002) demonstrated that antigen-engaged B cells have increased expression of CCR7 (OMIM Ref. No. 600242), the receptor for the T-zone chemokines CCL19 (OMIM Ref. No. 602227) and CCL21 (OMIM Ref. No. 602737), and that they exhibit increased responsiveness to both chemoattractants. In mice lacking lymphoid CCL19 and CCL21 chemokines, or with B cells that lack CCR7, antigen engagement fails to cause movement to the T zone. Using retroviral-mediated gene transfer, the authors demonstrated that increased expression of CCR7 is sufficient to direct B cells to the T zone. Reciprocally, overexpression of CXCR5, the receptor for the B-zone chemokine CXCL13, is sufficient to overcome antigen-induced B-cell movement to the T zone. Reif et al. (2002) concluded that their findings defined the mechanism of B-cell relocalization in response to antigen, and established that cell position in vivo can be determined by the balance of responsiveness to chemoattractants made in separate but adjacent zones. Animal model experiments lend further support to the function of CXCL13. CXC chemokine receptor-5 (CXCR5), the receptor for BLC, was known to be required for B-cell migration to splenic follicles, but the requirements for homing to B-cell areas and lymph nodes remained to be defined. Ansel et al. (2000) demonstrated that lymph nodes contain 2 types of B cell-rich compartment: follicles containing follicular dendritic cells, and areas lacking such cells. Ansel et al. (2000) generated mice deficient in BLC by targeted disruption. These mice had severe but incompletely penetrant defects in development of peripheral lymphoid or gans. Most mice lacked inguinal, iliac, sacral, brachial, and axillary lymph nodes, among others. However, most of these lymph nodes were found at varying low frequencies, and several other lymph nodes developed normally, with all animals possessing a full set of mesenteric lymph nodes. BLC-deficient mice were similar in appearance to mice deficient in CXCR5. However, CXCR5-deficient mice have less severe deficiency in Peyer patches. Using these BLC-deficient mice, Ansel et al. (2000) established that BLC and CXCR5 are needed for B-cell homing to follicles in lymph nodes as well as in spleen. They also found that BLC is required for the development of most lymph nodes and Peyer patches. In addition to mediating chemoattraction, BLC induces B cells to up-regulate membrane lymphotoxin alpha-1-beta-2 (see OMIM Ref. No. 600978), a cytokine that promotes follicular dendritic cell development and BLC expression, establishing a positive feedback loop thought to be important in follicle development and homeostasis. In germinal centers, the feedback loop is overridden, with B cell lymphotoxin alpha-1-beta-2 expression being induced by a mechanism independent of BLC.

It is appreciated that the abovementioned animal model for CXCL13 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ansel, K. M.; Harris, R. B. S.; Cyster, J. G.: CXCL13 is required for B1 cell homing, natural antibody production, and body cavity immunity. Immunity 16: 67-76, 2002.; and
Gunn, M. D.; Ngo, V. N.; Ansel, K. M.; Ekland, E. H.; Cyster, J. G.; Williams, L. T.: A B-cell-homing chemokine made in lymphoid follicles activates Burkitt's lymphoma receptor-1. Natu.

Further studies establishing the function and utilities of CXCL13 are found in John Hopkins OMIM database record ID 605149, and in sited publications numbered 1651, 2053-165 and 1796 listed in the bibliography section hereinbelow, which are also hereby incorporated by reference. RAS Guanyl Releasing Protein 1 (calcium and DAG-regulated) (RASGRP1, Accession NM_{—}005739) is another VGAM109 host target gene. RASGRP1 BINDING SITE is HOST TARGET binding site found in the 3′ untranslated region of mRNA encoded by RASGRP1, corresponding to a HOST TARGET binding site such as BINDING SITE I, BINDING SITE II or BINDING SITE III. Table 2 illustrates the complementarity of the nucleotide sequences of RASGRP1 BINDING SITE, designated SEQ ID:1241, to the nucleotide sequence of VGAM109 RNA, herein designated VGAM RNA, also designated SEQ ID:444.

Another function of VGAM109 is therefore inhibition of RAS Guanyl Releasing Protein 1 (calcium and DAG-regulated) (RASGRP1, Accession NM_{—}005739). Accordingly, utilities of VGAM109 include diagnosis, prevention and treatment of diseases and clinical conditions associated with RASGRP1. HTGN29 (Accession NM_{—}020199) is another VGAM109 host target gene. HTGN29 BINDING SITE is HOST TARGET binding site found in the 3′ untranslated region of mRNA encoded by HTGN29, corresponding to a HOST TARGET binding site such as BINDING SITE I, BINDING SITE II or BINDING SITE III. Table 2 illustrates the complementarity of the nucleotide sequences of HTGN29 BINDING SITE, designated SEQ ID:1899, to the nucleotide sequence of VGAM109 RNA, herein designated VGAM RNA, also designated SEQ ID:444.

Another function of VGAM109 is therefore inhibition of HTGN29 (Accession NM_{—}020199). Accordingly, utilities of VGAM109 include diagnosis, prevention and treatment of diseases and clinical conditions associated with HTGN29. KIAA1430 (Accession XM_{—}087593) is another VGAM109 host target gene. KIAA1430 BINDING SITE is HOST TARGET binding site found in the 3′ untranslated region of mRNA encoded by KIAA1430, corresponding to a HOST TARGET binding site such as BINDING SITE I, BINDING SITE II or BINDING SITE III. Table 2 illustrates the complementarity of the nucleotide sequences of KIAA1430 BINDING SITE, designated SEQ ID:3179, to the nucleotide sequence of VGAM109 RNA, herein designated VGAM RNA, also designated SEQ ID:444.

Another function of VGAM109 is therefore inhibition of KIAA1430 (Accession XM_{—}087593). Accordingly, utilities of VGAM109 include diagnosis, prevention and treatment of diseases and clinical conditions associated with KIAA1430. KIAA1764 (Accession XM_{—}045086) is another VGAM109 host target gene. KIAA1764 BINDING SITE is HOST TARGET binding site found in the 5′ untranslated region of mRNA encoded by KIAA1764, corresponding to a HOST TARGET binding site such as BINDING SITE I, BINDING SITE II or BINDING SITE III. Table 2 illustrates the complementarity of the nucleotide sequences of KIAA1764 BINDING SITE, designated SEQ ID:2850, to the nucleotide sequence of VGAM109 RNA, herein designated VGAM RNA, also designated SEQ ID:444.

Another function of VGAM109 is therefore inhibition of KIAA1764 (Accession XM_{—}045086). Accordingly, utilities of VGAM109 include diagnosis, prevention and treatment of diseases and clinical conditions associated with KIAA1764. ZAK (Accession NM_{—}133646) is another VGAM109 host target gene. ZAK BINDING SITE is HOST TARGET binding site found in the 3′ untranslated region of mRNA encoded by ZAK, corresponding to a HOST TARGET binding site such as BINDING SITE I, BINDING SITE II or BINDING SITE III. Table 2 illustrates the complementarity of the nucleotide sequences of ZAK BINDING SITE, designated SEQ ID:2428, to the nucleotide sequence of VGAM109 RNA, herein designated VGAM RNA, also designated SEQ ID:444.

Another function of VGAM109 is therefore inhibition of ZAK (Accession NM_{—}133646). Accordingly, utilities of VGAM109 include diagnosis, prevention and treatment of diseases and clinical conditions associated with ZAK. LOC151571 (Accession XM_{—}098088) is another VGAM109 host target gene. LOC151571 BINDING SITE is HOST TARGET binding site found in the 5′ untranslated region of mRNA encoded by LOC151571, corresponding to a HOST TARGET binding site such as BINDING SITE I, BINDING SITE II or BINDING SITE III. Table 2 illustrates the complementarity of the nucleotide sequences of LOC151571 BINDING SITE, designated SEQ ID:3348, to the nucleotide sequence of VGAM109 RNA, herein designated VGAM RNA, also designated SEQ ID:444.

Another function of VGAM109 is therefore inhibition of LOC151571 (Accession XM_{—}098088). Accordingly, utilities of VGAM109 include diagnosis, prevention and treatment of diseases and clinical conditions associated with LOC151571. LOC196989 (Accession XM_{—}116969) is another VGAM109 host target gene. LOC196989 BINDING SITE is HOST TARGET binding site found in the 3′ untranslated region of mRNA encoded by LOC196989, corresponding to a HOST TARGET binding site such as BINDING SITE I, BINDING SITE II or BINDING SITE III. Table 2 illustrates the complementarity of the nucleotide sequences of LOC196989 BINDING SITE, designated SEQ ID:3470, to the nucleotide sequence of VGAM109 RNA, herein designated VGAM RNA, also designated SEQ ID:444.

Another function of VGAM109 is therefore inhibition of LOC196989 (Accession XM_{—}116969). Accordingly, utilities of VGAM109 include diagnosis, prevention and treatment of diseases and clinical conditions associated with LOC196989. LOC221431 (Accession XM_{—}166380) is another VGAM109 host target gene. LOC221431 BINDING SITE is HOST TARGET binding site found in the 3′ untranslated region of mRNA encoded by LOC221431, corresponding to a HOST TARGET binding site such as BINDING SITE I, BINDING SITE II or BINDING SITE III. Table 2 illustrates the complementarity of the nucleotide sequences of LOC221431 BINDING SITE, designated SEQ ID:3553, to the nucleotide sequence of VGAM109 RNA, herein designated VGAM RNA, also designated SEQ ID:444.

Another function of VGAM109 is therefore inhibition of LOC221431 (Accession XM_{—}166380). Accordingly, utilities of VGAM109 include diagnosis, prevention and treatment of diseases and clinical conditions associated with LOC221431. FIG. 1 further provides a conceptual description of a novel bioinformatically detected viral gene of the present invention, referred to here as Viral Genomic Address Messenger 110 (VGAM110) viral gene, which modulates expression of respective host target genes thereof, the function and utility of which host target genes is known in the art.

VGAM110 is a novel bioinformatically detected regulatory, non protein coding, viral micro RNA (miRNA) gene. The method by which VGAM110 was detected is described hereinabove with reference to FIGS. 1-8.

VGAM110 gene, herein designated VGAM GENE, is a viral gene contained in the genome of Vaccinia Virus. VGAM110 host target gene, herein designated VGAM HOST TARGET GENE, is a human gene contained in the human genome.

VGAM110 gene encodes a VGAM110 precursor RNA, herein designated VGAM PRECURSOR RNA. Similar to other miRNA genes, and unlike most ordinary genes, VGAM110 precursor RNA does not encode a protein. A nucleotide sequence identical or highly similar to the nucleotide sequence of VGAM110 precursor RNA is designated SEQ ID:96, and is provided hereinbelow with reference to the sequence listing part. Nucleotide sequence SEQ ID:96 is located at position 51758 relative to the genome of Vaccinia Virus.

VGAM110 precursor RNA folds onto itself, forming VGAM110 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM110 folded precursor RNA into VGAM110 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM110 RNA is designated SEQ ID:445, and is provided hereinbelow with reference to the sequence listing part.

VGAM110 host target gene, herein designated VGAM HOST TARGET GENE, encodes a corresponding messenger RNA, VGAM110 host target RNA, herein designated VGAM HOST TARGET RNA. VGAM110 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM110 RNA, herein designated VGAM RNA, binds complementarily to one or more host target binding sites located in untranslated regions of VGAM110 host target RNA, herein designated VGAM HOST TARGET RNA. This complementary binding is due to the fact that the nucleotide sequence of VGAM110 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM110 RNA, herein designated VGAM RNA, may have a different number of host target binding sites in untranslated regions of a VGAM110 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM110 RNA, herein designated VGAM RNA, to host target binding sites on VGAM110 host target RNA, herein designated VGAM HOST TARGET RNA, such as BINDING SITE I, BINDING SITE II and BINDING SITE III, inhibits translation of VGAM110 host target RNA into VGAM110 host target protein, herein designated VGAM HOST TARGET PROTEIN. VGAM host target protein is therefore outlined by a broken line.

It is appreciated that VGAM110 host target gene, herein designated VGAM HOST TARGET GENE, in fact represents a plurality of VGAM110 host target genes. The mRNA of each one of this plurality of VGAM110 host target genes comprises one or more host target binding sites, each having a nucleotide sequence which is at least partly complementary to VGAM110 RNA, herein designated VGAM RNA, and which when bound by VGAM110 RNA causes inhibition of translation of respective one or more VGAM110 host target proteins.

It is further appreciated by one skilled in the art that the mode of translational inhibition illustrated by FIG. 1 with specific reference to translational inhibition exerted by VGAM110 gene, herein designated VGAM GENE, on one or more VGAM110 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM110 is inhibition of expression of host target genes, as part of a novel viral mechanism of attacking a host. Accordingly, utilities of VGAM110 include diagnosis, prevention and treatment of viral infection by Vaccinia Virus. Specific functions, and accordingly utilities, of VGAM110 correlate with, and may be deduced from, the identity of the host target genes which VGAM110 binds and inhibits, and the function of these host target genes, as elaborated hereinbelow.

Nucleotide sequences of the VGAM110 precursor RNA, herein designated VGAM PRECURSOR RNA, and of the ‘diced’ VGAM110 RNA, herein designated VGAM RNA, and a schematic representation of the secondary folding of VGAM110 folded precursor RNA, herein designated VGAM FOLDED PRECURSOR RNA, of VGAM110 are further described hereinbelow with reference to Table 1.

Nucleotide sequences of host target binding sites, such as BINDING SITE-I, BINDING SITE-II and BINDING SITE-III of FIG. 1, found on VGAM110 host target RNA, and schematic representation of the complementarity of each of these host target binding sites to VGAM110 RNA, herein designated VGAM RNA, are described hereinbelow with reference to Table 2.

As mentioned hereinabove with reference to FIG. 1, a function of VGAM110 gene, herein designated VGAM is inhibition of expression of VGAM110 target genes. It is appreciated that specific functions, and accordingly utilities, of VGAM110 correlate with, and may be deduced from, the identity of the target genes which VGAM110 binds and inhibits, and the function of these target genes, as elaborated hereinbelow.

Collagen, Type XII, Alpha 1 (COL12A1, Accession NM_{—}004370) is a VGAM110 host target gene. COL12A1 BINDING SITE1 and COL12A1 BINDING SITE2 are HOST TARGET binding sites found in untranslated regions of mRNA encoded by COL12A1, corresponding to HOST TARGET binding sites such as BINDING SITE I, BINDING SITE II or BINDING SITE III. Table 2 illustrates the complementarity of the nucleotide sequences of COL12A1 BINDING SITE1 and COL12A1 BINDING SITE2, designated SEQ ID:1102 and SEQ ID:2380 respectively, to the nucleotide sequence of VGAM110 RNA, herein designated VGAM RNA, also designated SEQ ID:445.

A function of VGAM110 is therefore inhibition of Collagen, Type XII, Alpha 1 (COL12A1, Accession NM_{—}004370). Accordingly, utilities of VGAM110 include diagnosis, prevention and treatment of diseases and clinical conditions associated with COL12A1. FIG. 1 further provides a conceptual description of a novel bioinformatically detected viral gene of the present invention, referred to here as Viral Genomic Address Messenger 111 (VGAM111) viral gene, which modulates expression of respective host target genes thereof, the function and utility of which host target genes is known in the art.

VGAM111 is a novel bioinformatically detected regulatory, non protein coding, viral micro RNA (miRNA) gene. The method by which VGAM111 was detected is described hereinabove with reference to FIGS. 1-8.

VGAM111 gene, herein designated VGAM GENE, is a viral gene contained in the genome of Vaccinia Virus. VGAM111 host target gene, herein designated VGAM HOST TARGET GENE, is a human gene contained in the human genome.

VGAM111 gene encodes a VGAM111 precursor RNA, herein designated VGAM PRECURSOR RNA. Similar to other miRNA genes, and unlike most ordinary genes, VGAM111 precursor RNA does not encode a protein. A nucleotide sequence identical or highly similar to the nucleotide sequence of VGAM111 precursor RNA is designated SEQ ID:97, and is provided hereinbelow with reference to the sequence listing part. Nucleotide sequence SEQ ID:97 is located at position 51252 relative to the genome of Vaccinia Virus.

VGAM111 precursor RNA folds onto itself, forming VGAM111 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM111 folded precursor RNA into VGAM111 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 25%) nucleotide sequence of VGAM111 RNA is designated SEQ ID:446, and is provided hereinbelow with reference to the sequence listing part.

VGAM111 host target gene, herein designated VGAM HOST TARGET GENE, encodes a corresponding messenger RNA, VGAM111 host target RNA, herein designated VGAM HOST TARGET RNA. VGAM111 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM111 RNA, herein designated VGAM RNA, binds complementarily to one or more host target binding sites located in untranslated regions of VGAM111 host target RNA, herein designated VGAM HOST TARGET RNA. This complementary binding is due to the fact that the nucleotide sequence of VGAM111 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM111 RNA, herein designated VGAM RNA, may have a different number of host target binding sites in untranslated regions of a VGAM111 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM111 RNA, herein designated VGAM RNA, to host target binding sites on VGAM111 host target RNA, herein designated VGAM HOST TARGET RNA, such as BINDING SITE I, BINDING SITE II and BINDING SITE III, inhibits translation of VGAM111 host target RNA into VGAM111 host target protein, herein designated VGAM HOST TARGET PROTEIN. VGAM host target protein is therefore outlined by a broken line.

It is appreciated that VGAM111 host target gene, herein designated VGAM HOST TARGET GENE, in fact represents a plurality of VGAM111 host target genes. The mRNA of each one of this plurality of VGAM111 host target genes comprises one or more host target binding sites, each having a nucleotide sequence which is at least partly complementary to VGAM111 RNA, herein designated VGAM RNA, and which when bound by VGAM111 RNA causes inhibition of translation of respective one or more VGAM111 host target proteins.

It is further appreciated by one skilled in the art that the mode of translational inhibition illustrated by FIG. 1 with specific reference to translational inhibition exerted by VGAM111 gene, herein designated VGAM GENE, on one or more VGAM111 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM111 is inhibition of expression of host target genes, as part of a novel viral mechanism of attacking a host. Accordingly, utilities of VGAM111 include diagnosis, prevention and treatment of viral infection by Vaccinia Virus. Specific functions, and accordingly utilities, of VGAM111 correlate with, and may be deduced from, the identity of the host target genes which VGAM111 binds and inhibits, and the function of these host target genes, as elaborated hereinbelow.

Nucleotide sequences of the VGAM111 precursor RNA, herein designated VGAM PRECURSOR RNA, and of the ‘diced’ VGAM111 RNA, herein designated VGAM RNA, and a schematic representation of the secondary folding of VGAM111 folded precursor RNA, herein designated VGAM FOLDED PRECURSOR RNA, of VGAM111 are further described hereinbelow with reference to Table 1.

Nucleotide sequences of host target binding sites, such as BINDING SITE-I, BINDING SITE-II and BINDING SITE-III of FIG. 1, found on VGAM111 host target RNA, and schematic representation of the complementarity of each of these host target binding sites to VGAM111 RNA, herein designated VGAM RNA, are described hereinbelow with reference to Table 2.

As mentioned hereinabove with reference to FIG. 1, a function of VGAM111 gene, herein designated VGAM is inhibition of expression of VGAM111 target genes. It is appreciated that specific functions, and accordingly utilities, of VGAM111 correlate with, and may be deduced from, the identity of the target genes which VGAM111 binds and inhibits, and the function of these target genes, as elaborated hereinbelow.

Ankyrin 1, Erythrocytic (ANK1, Accession NM_{—}000037) is a VGAM111 host target gene. ANK1 BINDING SITE1 and ANK1 BINDING SITE2 are HOST TARGET binding sites found in untranslated regions of mRNA encoded by ANK1, corresponding to HOST TARGET binding sites such as BINDING SITE I, BINDING SITE II or BINDING SITE III. Table 2 illustrates the complementarity of the nucleotide sequences of ANK1 BINDING SITE1 and ANK1 BINDING SITE2, designated SEQ ID:699 and SEQ ID:1918 respectively, to the nucleotide sequence of VGAM111 RNA, herein designated VGAM RNA, also designated SEQ ID:446.

A function of VGAM111 is therefore inhibition of Ankyrin 1, Erythrocytic (ANK1, Accession NM_{—}000037). Accordingly, utilities of VGAM111 include diagnosis, prevention and treatment of diseases and clinical conditions associated with ANK1. Desmoplakin (DPI, DPII) (DSP, Accession NM_{—}004415) is another VGAM111 host target gene. DSP BINDING SITE is HOST TARGET binding site found in the untranslated region of mRNA encoded by DSP, corresponding to a HOST TARGET binding site such as BINDING SITE I, BINDING SITE II or BINDING SITE III. Table 2 illustrates the complementarity of the nucleotide sequences of DSP BINDING SITE, designated SEQ ID:1110, to the nucleotide sequence of VGAM111 RNA, herein designated VGAM RNA, also designated SEQ ID:446.

Another function of VGAM111 is therefore inhibition of Desmoplakin (DPI, DPII) (DSP, Accession NM_{—}004415). Accordingly, utilities of VGAM111 include diagnosis, prevention and treatment of diseases and clinical conditions associated with DSP. Growth Differentiation Factor 5 (cartilage-derived morphogenetic protein-1) (GDF5, Accession NM_{—}000557) is another VGAM111 host target gene. GDF5 BINDING SITE is HOST TARGET binding site found in the 3′ untranslated region of mRNA encoded by GDF5, corresponding to a HOST TARGET binding site such as BINDING SITE I, BINDING SITE II or BINDING SITE III. Table 2 illustrates the complementarity of the nucleotide sequences of GDF5 BINDING SITE, designated SEQ ID:755, to the nucleotide sequence of VGAM111 RNA, herein designated VGAM RNA, also designated SEQ ID:446.

Another function of VGAM111 is therefore inhibition of Growth Differentiation Factor 5 (cartilage-derived morphogenetic protein-1) (GDF5, Accession NM_{—}000557), a gene which could be involved in bone formation. Accordingly, utilities of VGAM111 include diagnosis, prevention and treatment of diseases and clinical conditions associated with GDF5. The function of GDF5 has been established by previous studies. Thomas et al. (1997) showed that a cys400-to-tyr mutation (C400Y; 601146.0003) in the CDMP1 gene resulted in Grebe type chondrodysplasia (OMIM Ref. No. 200700). They found that the mutant protein is not secreted and is inactive in vitro. It produced a dominant-negative effect by preventing the secretion of other, related bone morphogenetic proteins (BMPs). This appeared to occur through the formation of heterodimers. The mutation and its proposed mechanism of action provided the first human genetic indication that composite expression patterns of different BMPs dictate limb and digit morphogenesis. The role of a dominant-negative mutation in a recessive disorder was illustrated. Animal model experiments lend further support to the function of GDF5. Tsumaki et al. (1999) generated transgenic mice expressing recombinant CDMP1. These mice died before or just after birth and exhibited chondrodysplasia with expanded primordial cartilage, which consisted of an enlarged hypertrophic zone and a reduced proliferating chondrocyte zone, not only in the limbs but also in the axial skeleton. Histologically, CDMP1 increased the number of chondroprogenitor cells and accelerated chondrocyte differentiation to hypertrophy. Moreover, ectopic expression of CDMP1 in the notochord before onset of chondrogenesis inhibited mesenchymal cell condensation around the notochord, which led to failure of vertebral body formation.

It is appreciated that the abovementioned animal model for GDF5 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Thomas, J. T.; Kilpatrick, M. W.; Lin, K.; Erlacher, L.; Lembessis, P.; Costa, T.; Tsipouras, P.; Luyten, F. P.: Disruption of human limb morphogenesis by a dominant negative mutation in CDMP1. Nature Genet. 58-64, 1997.; and
Tsumaki, N.; Tanaka, K.; Arikawa-Hirasawa, E.; Nakase, T.; Kimura, T.; Thomas, J. T.; Ochi, T.; Luyten, F. P.; Yamada, Y.: Role of CDMP-1 in skeletal morphogenesis: promotion of mesenc.

Further studies establishing the function and utilities of GDF5 are found in John Hopkins OMIM database record ID 601146, and in sited publications numbered 2080, 2083, 2134-1106, 2283, 1700, 1107-1108, 1701, 1702, 271 and 2791 listed in the bibliography section hereinbelow, which are also hereby incorporated by reference. Oxoglutarate (alpha-ketoglutarate) Dehydrogenase (lipoamide) (OGDH, Accession XM_{—}165877) is another VGAM111 host target gene. OGDH BINDING SITE is HOST TARGET binding site found in the 3′ untranslated region of mRNA encoded by OGDH, corresponding to a HOST TARGET binding site such as BINDING SITE I, BINDING SITE II or BINDING SITE III. Table 2 illustrates the complementarity of the nucleotide sequences of OGDH BINDING SITE, designated SEQ ID:3510, to the nucleotide sequence of VGAM111 RNA, herein designated VGAM RNA, also designated SEQ ID:446.

Another function of VGAM111 is therefore inhibition of Oxoglutarate (alpha-ketoglutarate) Dehydrogenase (lipoamide) (OGDH, Accession XM_{—}165877), a gene which helps convert a-ketoglutarate to succinyl coenzyme A in Krebs cycle. Accordingly, utilities of VGAM111 include diagnosis, prevention and treatment of diseases and clinical conditions associated with OGDH. The function of OGDH has been established by previous studies. In 3 males born to first-cousin Algerian parents and presenting with hypotonia, metabolic acidosis, and hyperlactatemia immediately after birth, Bonnefont et al. (1992) described a ‘new’ inborn error of the tricarboxylic acid cycle, alpha-ketoglutarate dehydrogenase deficiency. Neurologic deterioration resulted in death at about 30 months of age. Low molar ratios of ketone bodies in plasma of neonates with congenital lactic acidosis were proposed as an indicator of dysfunction of the tricarboxylic acid cycle. The parents of the 3 sibs were related as first cousins. Kohlschutter et al. (1982) reported a case of partial alpha-ketoglutarate dehydrogenase deficiency in 2 sibs of a consanguineous Tunisian family. In addition to genetic defects of the tricarboxylic acid cycle, other mechanisms for recessively inherited congenital lactic acidosis include inborn errors of pyruvate metabolism and inborn errors of oxidative phosphorylation. Guffon et al. (1993) described 2 new familial cases of 2-ketoglutarate dehydrogenase deficiency: a sister and brother born of consanguineous Portuguese parents. Axial hypotonia with no head control as late as age 4 years was described in both. Metabolic acidosis with acute episodes of acidotic decompensation and sometimes hypoglycemia occurred during infections. The sister died suddenly after a general anesthesia. The alpha-ketoglutarate dehydrogenase complex is a multienzyme complex consisting of 3 protein subunits, alpha-ketoglutarate dehydrogenase (Elk, or oxoglutarate dehydrogenase; OGDH; EC 1.2.4.2), dihydrolipoyl succinyl-transferase (E2k, or DLST; 126063), and dihydrolipoyl dehydrogenase (E3; 246900). The complex catalyzes a key reaction in the Krebs tricarboxylic acid cycle. The DLST gene maps to 14q24.3 and the E3 gene maps to chromosome 7. Koike et al. (1992) cloned the Elk gene, which codes for the alpha-ketoglutarate dehydrogenase subunit. Using 2 human/rodent somatic cell hybrid panels, Szabo et al. (1994) mapped the OGDH gene to 7p13-p11.2. A second related sequence, possibly a pseudogene, was identified and mapped to chromosome 10. Szabo et al. (1994) pointed to a possible significance to the finding of a reduction in the activity of this complex in Alzheimer disease brain and cultured skin fibroblasts from Alzheimer disease patients.

Full details of the abovementioned studies are described in the following publications, the disclosure of which are hereby incorporated by reference:
Guffon, N.; Lopez-Mediavilla, C.; Dumoulin, R.; Mousson, B.; Godinot, C.; Carrier, H.; Collombet, J. M.; Divry, P.; Mathieu, M.; Guibaud, P.: 2-Ketoglutarate dehydrogenase deficiency, a rare cause of primary hyperlactataemia: report of a new case. J. Inherit. Metab. Dis. 16: 821-830, 1993.; and
Szabo, P.; Cai, X.; Ali, G.; Blass, J. P.: Localization of the gene (OGDH) coding for the Elk component of the alpha-ketoglutarate dehydrogenase complex to chromosome 7p13-p11.2. Genomi.

Further studies establishing the function and utilities of OGDH are found in John Hopkins OMIM database record ID 203740, and in sited publications numbered 2973-2978 listed in the bibliography section hereinbelow, which are also hereby incorporated by reference. Regulatory Factor X, 2 (influences HLA class II expression) (RFX2, Accession NM_{—}000635) is another VGAM111 host target gene. RFX2 BINDING SITE is HOST TARGET binding site found in the 3′ untranslated region of mRNA encoded by RFX2, corresponding to a HOST TARGET binding site such as BINDING SITE I, BINDING SITE II or BINDING SITE III. Table 2 illustrates the complementarity of the nucleotide sequences of RFX2 BINDING SITE, designated SEQ ID:765, to the nucleotide sequence of VGAM111 RNA, herein designated VGAM RNA, also designated SEQ ID:446.

Another function of VGAM111 is therefore inhibition of Regulatory Factor X, 2 (influences HLA class II expression) (RFX2, Accession NM_{—}000635), a gene which acts as a dimer to regulate the expression of many genes. Accordingly, utilities of VGAM111 include diagnosis, prevention and treatment of diseases and clinical conditions associated with RFX2. The function of RFX2 has been established by previous studies. Pugliatti et al. (1992) noted that the expression of HLA class II genes (DR, DQ, and DP) is controlled primarily by cis-acting DNA motifs located within the 150 bp upstream of the genes and in particular by 2 highly conserved sequences, the X and Y boxes. Several protein factors bind to these cis-acting sequences. RFX is of special interest since a specific defect in its binding to target DNA sequence has been observed in patients with HLA class II deficient combined immunodeficiency (see OMIM Ref. No. 209920). Overexpression of RFX in transfected cells transactivates an HLA class II promoter, and antisense RNA expressed in transfected cells inhibits the expression of HLA-DR genes. A closely related form of RFX, referred to as RFX2, has also been isolated. The RFX1 (OMIM Ref. No. 600006) gene product is a transactivator of the human hepatitis B viral enhancer 1. Reith et al. (1994) commented that the RFX family members, particularly RFX1 and RFX3 (OMIM Ref. No. 601337), constitute the nuclear complexes referred to previously as enhancer factor C (EF-C), EP, and methylation-dependent DNA-binding protein (MDBP), or rpL30-alpha. Reith et al. (1994) identified and cloned 3 members of this gene family from both human and mouse using lambda gt11 cDNA libraries. The gene encoding human RFX2 encodes a 721-amino acid polypeptide. Homology between the 3 RFX proteins is restricted largely to 5 conserved regions, including the 2 domains required for DNA binding and dimerization. Reith et al. (1994) found that RFX1, RFX2, and RFX3 have similar DNA-binding specificities. The RFX monomers can heterodimerize both in vivo and in vitro, but all 3 are capable of binding DNA as monomers. They showed that the RFX2 transcript is particularly elevated in mouse testis.

Full details of the abovementioned studies are described in the following publications, the disclosure of which are hereby incorporated by reference:
Pugliatti, L.; Derre, J.; Berger, R.; Ucla, C.; Reith, W.; Mach, B.: The genes for MHC class II regulatory factors RFX1 and RFX2 are located on the short arm of chromosome 19. Genomics 13: 1307-1310, 1992.; and
Reith, W.; Ucla, C.; Barras, E.; Gaud, A.; Durand, B.; Herrero-Sanchez, C.; Kobr, M.; Mach, B.: RFX1, a transactivator of hepatitis B virus enhancer 1, belongs to a novel family of hom.

Further studies establishing the function and utilities of RFX2 are found in John Hopkins OMIM database record ID 142765, and in sited publications numbered 1061-1063 listed in the bibliography section hereinbelow, which are also hereby incorporated by reference. SET7 (Accession NM_{—}030648) is another VGAM111 host target gene. SET7 BINDING SITE is HOST TARGET binding site found in the 5′ untranslated region of mRNA encoded by SET7, corresponding to a HOST TARGET binding site such as BINDING SITE I, BINDING SITE II or BINDING SITE III. Table 2 illustrates the complementarity of the nucleotide sequences of SET7 BINDING SITE, designated SEQ ID:2153, to the nucleotide sequence of VGAM111 RNA, herein designated VGAM RNA, also designated SEQ ID:446.

Another function of VGAM111 is therefore inhibition of SET7 (Accession NM_{—}030648). Accordingly, utilities of VGAM111 include diagnosis, prevention and treatment of diseases and clinical conditions associated with SET7. Chromosome 17 Open Reading Frame 31 (C17orf31, Accession NM_{—}017575) is another VGAM111 host target gene. C17orf31 BINDING SITE is HOST TARGET binding site found in the 3′ untranslated region of mRNA encoded by C17orf31, corresponding to a HOST TARGET binding site such as BINDING SITE I, BINDING SITE II or BINDING SITE III. Table 2 illustrates the complementarity of the nucleotide sequences of C17orf31 BINDING SITE, designated SEQ ID:1723, to the nucleotide sequence of VGAM111 RNA, herein designated VGAM RNA, also designated SEQ ID:446.

Another function of VGAM111 is therefore inhibition of Chromosome 17 Open Reading Frame 31 (C17orf31, Accession NM_{—}017575). Accordingly, utilities of VGAM111 include diagnosis, prevention and treatment of diseases and clinical conditions associated with C17orf31. caspr5 (Accession NM_{—}130773) is another VGAM111 host target gene. caspr5 BINDING SITE1 and caspr5 BINDING SITE2 are HOST TARGET binding sites found in untranslated regions of mRNA encoded by caspr5, corresponding to HOST TARGET binding sites such as BINDING SITE I, BINDING SITE II or BINDING SITE III. Table 2 illustrates the complementarity of the nucleotide sequences of caspr5 BINDING SITE1 and caspr5 BINDING SITE2, designated SEQ ID:2396 and SEQ ID:2462 respectively, to the nucleotide sequence of VGAM111 RNA, herein designated VGAM RNA, also designated SEQ ID:446.

Another function of VGAM111 is therefore inhibition of caspr5 (Accession NM_{—}130773). Accordingly, utilities of VGAM111 include diagnosis, prevention and treatment of diseases and clinical conditions associated with caspr5. DKFZP434P0111 (Accession XM_{—}041116) is another VGAM111 host target gene. DKFZP434P0111 BINDING SITE is HOST TARGET binding site found in the 3′ untranslated region of mRNA encoded by DKFZP434P0111, corresponding to a HOST TARGET binding site such as BINDING SITE I, BINDING SITE II or BINDING SITE III. Table 2 illustrates the complementarity of the nucleotide sequences of DKFZP434P0111 BINDING SITE, designated SEQ ID:2790, to the nucleotide sequence of VGAM111 RNA, herein designated VGAM RNA, also designated SEQ ID:446.

Another function of VGAM111 is therefore inhibition of DKFZP434P0111 (Accession XM_{—}041116). Accordingly, utilities of VGAM111 include diagnosis, prevention and treatment of diseases and clinical conditions associated with DKFZP434P0111. FLJ10634 (Accession NM_{—}018163) is another VGAM111 host target gene. FLJ10634 BINDING SITE is HOST TARGET binding site found in the 3′ untranslated region of mRNA encoded by FLJ10634, corresponding to a HOST TARGET binding site such as BINDING SITE I, BINDING SITE II or BINDING SITE III. Table 2 illustrates the complementarity of the nucleotide sequences of FLJ10634 BINDING SITE, designated SEQ ID:1791, to the nucleotide sequence of VGAM111 RNA, herein designated VGAM RNA, also designated SEQ ID:446.

Another function of VGAM111 is therefore inhibition of FLJ10634 (Accession NM_{—}018163). Accordingly, utilities of VGAM111 include diagnosis, prevention and treatment of diseases and clinical conditions associated with FLJ10634. FLJ32334 (Accession NM_{—}144565) is another VGAM111 host target gene. FLJ32334 BINDING SITE is HOST TARGET binding site found in the 3′ untranslated region of mRNA encoded by FLJ32334, corresponding to a HOST TARGET binding site such as BINDING SITE I, BINDING SITE II or BINDING SITE III. Table 2 illustrates the complementarity of the nucleotide sequences of FLJ32334 BINDING SITE, designated SEQ ID:2483, to the nucleotide sequence of VGAM111 RNA, herein designated VGAM RNA, also designated SEQ ID:446.

Another function of VGAM111 is therefore inhibition of FLJ32334 (Accession NM_{—}144565). Accordingly, utilities of VGAM111 include diagnosis, prevention and treatment of diseases and clinical conditions associated with FLJ32334. FLJ32894 (Accession NM_{—}144667) is another VGAM111 host target gene. FLJ32894 BINDING SITE is HOST TARGET binding site found in the 3′ untranslated region of mRNA encoded by FLJ32894, corresponding to a HOST TARGET binding site such as BINDING SITE I, BINDING SITE II or BINDING SITE III. Table 2 illustrates the complementarity of the nucleotide sequences of FLJ32894 BINDING SITE, designated SEQ ID:2491, to the nucleotide sequence of VGAM111 RNA, herein designated VGAM RNA, also designated SEQ ID:446.

Another function of VGAM111 is therefore inhibition of FLJ32894 (Accession NM_{—}144667). Accordingly, utilities of VGAM111 include diagnosis, prevention and treatment of diseases and clinical conditions associated with FLJ32894. KIAA0711 (Accession NM_{—}014867) is another VGAM111 host target gene. KIAA0711 BINDING SITE is HOST TARGET binding site found in the 5′ untranslated region of mRNA encoded by KIAA0711, corresponding to a HOST TARGET binding site such as BINDING SITE I, BINDING SITE II or BINDING SITE III. Table 2 illustrates the complementarity of the nucleotide sequences of KIAA0711 BINDING SITE, designated SEQ ID:1572, to the nucleotide sequence of VGAM111 RNA, herein designated VGAM RNA, also designated SEQ ID:446.

Another function of VGAM111 is therefore inhibition of KIAA0711 (Accession NM_{—}014867). Accordingly, utilities of VGAM111 include diagnosis, prevention and treatment of diseases and clinical conditions associated with KIAA0711. KIAA1649 (Accession NM_{—}032311) is another VGAM111 host target gene. KIAA1649 BINDING SITE1 and KIAA1649 BINDING SITE2 are HOST TARGET binding sites found in untranslated regions of mRNA encoded by KIAA1649, corresponding to HOST TARGET binding sites such as BINDING SITE I, BINDING SITE II or BINDING SITE III. Table 2 illustrates the complementarity of the nucleotide sequences of KIAA1649 BINDING SITE1 and KIAA1649 BINDING SITE2, designated SEQ ID:2245 and SEQ ID:2770 respectively, to the nucleotide sequence of VGAM111 RNA, herein designated VGAM RNA, also designated SEQ ID:446.

Another function of VGAM111 is therefore inhibition of KIAA1649 (Accession NM_{—}032311). Accordingly, utilities of VGAM111 include diagnosis, prevention and treatment of diseases and clinical conditions associated with KIAA1649. MGC4737 (Accession NM_{—}031466) is another VGAM111 host target gene. MGC4737 BINDING SITE is HOST TARGET binding site found in the 3′ untranslated region of mRNA encoded by MGC4737, corresponding to a HOST TARGET binding site such as BINDING SITE I, BINDING SITE II or BINDING SITE III. Table 2 illustrates the complementarity of the nucleotide sequences of MGC4737 BINDING SITE, designated SEQ ID:2206, to the nucleotide sequence of VGAM111 RNA, herein designated VGAM RNA, also designated SEQ ID:446.

Another function of VGAM111 is therefore inhibition of MGC4737 (Accession NM_{—}031466). Accordingly, utilities of VGAM111 include diagnosis, prevention and treatment of diseases and clinical conditions associated with MGC4737. LOC115110 (Accession XM_{—}049825) is another VGAM111 host target gene. LOC115110 BINDING SITE is HOST TARGET binding site found in the 5′ untranslated region of mRNA encoded by LOC115110, corresponding to a HOST TARGET binding site such as BINDING SITE I, BINDING SITE II or BINDING SITE III. Table 2 illustrates the complementarity of the nucleotide sequences of LOC115110 BINDING SITE, designated SEQ ID:2923, to the nucleotide sequence of VGAM111 RNA, herein designated VGAM RNA, also designated SEQ ID:446.

Another function of VGAM111 is therefore inhibition of LOC115110 (Accession XM_{—}049825). Accordingly, utilities of VGAM111 include diagnosis, prevention and treatment of diseases and clinical conditions associated with LOC115110. LOC123242 (Accession XM_{—}063548) is another VGAM111 host target gene. LOC123242 BINDING SITE is HOST TARGET binding site found in the 5′ untranslated region of mRNA encoded by LOC123242, corresponding to a HOST TARGET binding site such as BINDING SITE I, BINDING SITE II or BINDING SITE III. Table 2 illustrates the complementarity of the nucleotide sequences of LOC123242 BINDING SITE, designated SEQ ID:3029, to the nucleotide sequence of VGAM111 RNA, herein designated VGAM RNA, also designated SEQ ID:446.

Another function of VGAM111 is therefore inhibition of LOC123242 (Accession XM_{—}063548). Accordingly, utilities of VGAM111 include diagnosis, prevention and treatment of diseases and clinical conditions associated with LOC123242. LOC146237 (Accession XM_{—}096954) is another VGAM111 host target gene. LOC146237 BINDING SITE is HOST TARGET binding site found in the 3′ untranslated region of mRNA encoded by LOC146237, corresponding to a HOST TARGET binding site such as BINDING SITE I, BINDING SITE II or BINDING SITE III. Table 2 illustrates the complementarity of the nucleotide sequences of LOC146237 BINDING SITE, designated SEQ ID:3282, to the nucleotide sequence of VGAM111 RNA, herein designated VGAM RNA, also designated SEQ ID:446.

Another function of VGAM111 is therefore inhibition of LOC146237 (Accession XM_{—}096954). Accordingly, utilities of VGAM111 include diagnosis, prevention and treatment of diseases and clinical conditions associated with LOC146237. LOC147160 (Accession XM_{—}097202) is another VGAM111 host target gene. LOC147160 BINDING SITE is HOST TARGET binding site found in the 5′ untranslated region of mRNA encoded by LOC147160, corresponding to a HOST TARGET binding site such as BINDING SITE I, BINDING SITE II or BINDING SITE III. Table 2 illustrates the complementarity of the nucleotide sequences of LOC147160 BINDING SITE, designated SEQ ID:3291, to the nucleotide sequence of VGAM111 RNA, herein designated VGAM RNA, also designated SEQ ID:446.

Another function of VGAM111 is therefore inhibition of LOC147160 (Accession XM_{—}097202). Accordingly, utilities of VGAM111 include diagnosis, prevention and treatment of diseases and clinical conditions associated with LOC147160. LOC149670 (Accession XM_{—}086647) is another VGAM111 host target gene. LOC149670 BINDING SITE is HOST TARGET binding site found in the 5′ untranslated region of mRNA encoded by LOC149670, corresponding to a HOST TARGET binding site such as BINDING SITE I, BINDING SITE II or BINDING SITE III. Table 2 illustrates the complementarity of the nucleotide sequences of LOC149670 BINDING SITE, designated SEQ ID:3138, to the nucleotide sequence of VGAM111 RNA, herein designated VGAM RNA, also designated SEQ ID:446.

Another function of VGAM111 is therefore inhibition of LOC149670 (Accession XM_{—}086647). Accordingly, utilities of VGAM111 include diagnosis, prevention and treatment of diseases and clinical conditions associated with LOC149670. LOC155072 (Accession XM_{—}098661) is another VGAM111 host target gene. LOC155072 BINDING SITE is HOST TARGET binding site found in the 3′ untranslated region of mRNA encoded by LOC155072, corresponding to a HOST TARGET binding site such as BINDING SITE I, BINDING SITE II or BINDING SITE III. Table 2 illustrates the complementarity of the nucleotide sequences of LOC155072 BINDING SITE, designated SEQ ID:3374, to the nucleotide sequence of VGAM111 RNA, herein designated VGAM RNA, also designated SEQ ID:446.

Another function of VGAM111 is therefore inhibition of LOC155072 (Accession XM_{—}098661). Accordingly, utilities of VGAM111 include diagnosis, prevention and treatment of diseases and clinical conditions associated with LOC155072. LOC158828 (Accession XM_{—}088689) is another VGAM111 host target gene. LOC158828 BINDING SITE is HOST TARGET binding site found in the 3′ untranslated region of mRNA encoded by LOC158828, corresponding to a HOST TARGET binding site such as BINDING SITE I, BINDING SITE II or BINDING SITE III. Table 2 illustrates the complementarity of the nucleotide sequences of LOC158828 BINDING SITE, designated SEQ ID:3227, to the nucleotide sequence of VGAM111 RNA, herein designated VGAM RNA, also designated SEQ ID:446.

Another function of VGAM111 is therefore inhibition of LOC158828 (Accession XM_{—}088689). Accordingly, utilities of VGAM111 include diagnosis, prevention and treatment of diseases and clinical conditions associated with LOC158828. LOC202868 (Accession XM_{—}117477) is another VGAM111 host target gene. LOC202868 BINDING SITE is HOST TARGET binding site found in the 3′ untranslated region of mRNA encoded by LOC202868, corresponding to a HOST TARGET binding site such as BINDING SITE I, BINDING SITE II or BINDING SITE III. Table 2 illustrates the complementarity of the nucleotide sequences of LOC202868 BINDING SITE, designated SEQ ID:3487, to the nucleotide sequence of VGAM111 RNA, herein designated VGAM RNA, also designated SEQ ID:446.

Another function of VGAM111 is therefore inhibition of LOC202868 (Accession XM_{—}117477). Accordingly, utilities of VGAM111 include diagnosis, prevention and treatment of diseases and clinical conditions associated with LOC202868. LOC253001 (Accession XM_{—}171711) is another VGAM111 host target gene. LOC253001 BINDING SITE is HOST TARGET binding site found in the 5′ untranslated region of mRNA encoded by LOC253001, corresponding to a HOST TARGET binding site such as BINDING SITE I, BINDING SITE II or BINDING SITE III. Table 2 illustrates the complementarity of the nucleotide sequences of LOC253001 BINDING SITE, designated SEQ ID:3697, to the nucleotide sequence of VGAM111 RNA, herein designated VGAM RNA, also designated SEQ ID:446.

Another function of VGAM111 is therefore inhibition of LOC253001 (Accession XM_{—}171711). Accordingly, utilities of VGAM111 include diagnosis, prevention and treatment of diseases and clinical conditions associated with LOC253001. LOC253841 (Accession XM_{—}172811) is another VGAM111 host target gene. LOC253841 BINDING SITE is HOST TARGET binding site found in the 5′ untranslated region of mRNA encoded by LOC253841, corresponding to a HOST TARGET binding site such as BINDING SITE I, BINDING SITE II or BINDING SITE III. Table 2 illustrates the complementarity of the nucleotide sequences of LOC253841 BINDING SITE, designated SEQ ID:3704, to the nucleotide sequence of VGAM111 RNA, herein designated VGAM RNA, also designated SEQ ID:446.

Another function of VGAM111 is therefore inhibition of LOC253841 (Accession XM_{—}172811). Accordingly, utilities of VGAM111 include diagnosis, prevention and treatment of diseases and clinical conditions associated with LOC253841. LOC51177 (Accession NM_{—}016274) is another VGAM111 host target gene. LOC51177 BINDING SITE is HOST TARGET binding site found in the 5′ untranslated region of mRNA encoded by LOC51177, corresponding to a HOST TARGET binding site such as BINDING SITE I, BINDING SITE II or BINDING SITE III. Table 2 illustrates the complementarity of the nucleotide sequences of LOC51177 BINDING SITE, designated SEQ ID:1676, to the nucleotide sequence of VGAM111 RNA, herein designated VGAM RNA, also designated SEQ ID:446.

Another function of VGAM111 is therefore inhibition of LOC51177 (Accession NM_{—}016274). Accordingly, utilities of VGAM111 include diagnosis, prevention and treatment of diseases and clinical conditions associated with LOC51177. LOC91408 (Accession XM_{—}038290) is another VGAM111 host target gene. LOC91408 BINDING SITE is HOST TARGET binding site found in the 3′ untranslated region of mRNA encoded by LOC91408, corresponding to a HOST TARGET binding site such as BINDING SITE I, BINDING SITE II or BINDING SITE III. Table 2 illustrates the complementarity of the nucleotide sequences of LOC91408 BINDING SITE, designated SEQ ID:2735, to the nucleotide sequence of VGAM111 RNA, herein designated VGAM RNA, also designated SEQ ID:446.

Another function of VGAM111 is therefore inhibition of LOC91408 (Accession XM_{—}038290). Accordingly, utilities of VGAM111 include diagnosis, prevention and treatment of diseases and clinical conditions associated with LOC91408. FIG. 1 further provides a conceptual description of a novel bioinformatically detected viral gene of the present invention, referred to here as Viral Genomic Address Messenger 112 (VGAM112) viral gene, which modulates expression of respective host target genes thereof, the function and utility of which host target genes is known in the art.

VGAM112 is a novel bioinformatically detected regulatory, non protein coding, viral micro RNA (miRNA) gene. The method by which VGAM112 was detected is described hereinabove with reference to FIGS. 1-8.

VGAM112 gene, herein designated VGAM GENE, is a viral gene contained in the genome of Vaccinia Virus. VGAM112 host target gene, herein designated VGAM HOST TARGET GENE, is a human gene contained in the human genome.

VGAM112 gene encodes a VGAM112 precursor RNA, herein designated VGAM PRECURSOR RNA. Similar to other miRNA genes, and unlike most ordinary genes, VGAM112 precursor RNA does not encode a protein. A nucleotide sequence identical or highly similar to the nucleotide sequence of VGAM112 precursor RNA is designated SEQ ID:98, and is provided hereinbelow with reference to the sequence listing part. Nucleotide sequence SEQ ID:98 is located at position 50148 relative to the genome of Vaccinia Virus.

VGAM112 precursor RNA folds onto itself, forming VGAM112 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM112 folded precursor RNA into VGAM112 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM112 RNA is designated SEQ ID:447, and is provided hereinbelow with reference to the sequence listing part.

VGAM112 host target gene, herein designated VGAM HOST TARGET GENE, encodes a corresponding messenger RNA, VGAM112 host target RNA, herein designated VGAM HOST TARGET RNA. VGAM112 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM112 RNA, herein designated VGAM RNA, binds complementarily to one or more host target binding sites located in untranslated regions of VGAM112 host target RNA, herein designated VGAM HOST TARGET RNA. This complementary binding is due to the fact that the nucleotide sequence of VGAM112 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM112 RNA, herein designated VGAM RNA, may have a different number of host target binding sites in untranslated regions of a VGAM112 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM112 RNA, herein designated VGAM RNA, to host target binding sites on VGAM112 host target RNA, herein designated VGAM HOST TARGET RNA, such as BINDING SITE I, BINDING SITE II and BINDING SITE III, inhibits translation of VGAM112 host target RNA into VGAM112 host target protein, herein designated VGAM HOST TARGET PROTEIN. VGAM host target protein is therefore outlined by a broken line.

It is appreciated that VGAM112 host target gene, herein designated VGAM HOST TARGET GENE, in fact represents a plurality of VGAM112 host target genes. The mRNA of each one of this plurality of VGAM112 host target genes comprises one or more host target binding sites, each having a nucleotide sequence which is at least partly complementary to VGAM112 RNA, herein designated VGAM RNA, and which when bound by VGAM112 RNA causes inhibition of translation of respective one or more VGAM112 host target proteins.

It is further appreciated by one skilled in the art that the mode of translational inhibition illustrated by FIG. 1 with specific reference to translational inhibition exerted by VGAM112 gene, herein designated VGAM GENE, on one or more VGAM112 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM112 is inhibition of expression of host target genes, as part of a novel viral mechanism of attacking a host. Accordingly, utilities of VGAM112 include diagnosis, prevention and treatment of viral infection by Vaccinia Virus. Specific functions, and accordingly utilities, of VGAM112 correlate with, and may be deduced from, the identity of the host target genes which VGAM112 binds and inhibits, and the function of these host target genes, as elaborated hereinbelow.

Nucleotide sequences of the VGAM112 precursor RNA, herein designated VGAM PRECURSOR RNA, and of the ‘diced’ VGAM112 RNA, herein designated VGAM RNA, and a schematic representation of the secondary folding of VGAM112 folded precursor RNA, herein designated VGAM FOLDED PRECURSOR RNA, of VGAM112 are further described hereinbelow with reference to Table 1.

Nucleotide sequences of host target binding sites, such as BINDING SITE-I, BINDING SITE-II and BINDING SITE-III of FIG. 1, found on VGAM112 host target RNA, and schematic representation of the complementarity of each of these host target binding sites to VGAM112 RNA, herein designated VGAM RNA, are described hereinbelow with reference to Table 2.

As mentioned hereinabove with reference to FIG. 1, a function of VGAM112 gene, herein designated VGAM is inhibition of expression of VGAM112 target genes. It is appreciated that specific functions, and accordingly utilities, of VGAM112 correlate with, and may be deduced from, the identity of the target genes which VGAM112 binds and inhibits, and the function of these target genes, as elaborated hereinbelow.

KIAA1265 (Accession XM_{—}047707) is a VGAM112 host target gene. KIAA1265 BINDING SITE is HOST TARGET binding site found in the 3′ untranslated region of mRNA encoded by KIAA1265, corresponding to a HOST TARGET binding site such as BINDING SITE I, BINDING SITE II or BINDING SITE III. Table 2 illustrates the complementarity of the nucleotide sequences of KIAA1265 BINDING SITE, designated SEQ ID:2896, to the nucleotide sequence of VGAM112 RNA, herein designated VGAM RNA, also designated SEQ ID:447.

A function of VGAM112 is therefore inhibition of KIAA1265 (Accession XM_{—}047707). Accordingly, utilities of VGAM112 include diagnosis, prevention and treatment of diseases and clinical conditions associated with KIAA1265. MGC2731 (Accession NM_{—}024068) is another VGAM112 host target gene. MGC2731 BINDING SITE is HOST TARGET binding site found in the 3′ untranslated region of mRNA encoded by MGC2731, corresponding to a HOST TARGET binding site such as BINDING SITE I, BINDING SITE II or BINDING SITE III. Table 2 illustrates the complementarity of the nucleotide sequences of MGC2731 BINDING SITE, designated SEQ ID:2050, to the nucleotide sequence of VGAM112 RNA, herein designated VGAM RNA, also designated SEQ ID:447.

Another function of VGAM112 is therefore inhibition of MGC2731 (Accession NM_{—}024068). Accordingly, utilities of VGAM112 include diagnosis, prevention and treatment of diseases and clinical conditions associated with MGC2731. FIG. 1 further provides a conceptual description of a novel bioinformatically detected viral gene of the present invention, referred to here as Viral Genomic Address Messenger 113 (VGAM113) viral gene, which modulates expression of respective host target genes thereof, the function and utility of which host target genes is known in the art.

VGAM113 is a novel bioinformatically detected regulatory, non protein coding, viral micro RNA (miRNA) gene. The method by which VGAM113 was detected is described hereinabove with reference to FIGS. 1-8.

VGAM113 gene, herein designated VGAM GENE, is a viral gene contained in the genome of Vaccinia Virus. VGAM113 host target gene, herein designated VGAM HOST TARGET GENE, is a human gene contained in the human genome.

VGAM113 gene encodes a VGAM113 precursor RNA, herein designated VGAM PRECURSOR RNA. Similar to other miRNA genes, and unlike most ordinary genes, VGAM113 precursor RNA does not encode a protein. A nucleotide sequence identical or highly similar to the nucleotide sequence of VGAM113 precursor RNA is designated SEQ ID:99, and is provided hereinbelow with reference to the sequence listing part. Nucleotide sequence SEQ ID:99 is located at position 49143 relative to the genome of Vaccinia Virus.

VGAM113 precursor RNA folds onto itself, forming VGAM113 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM113 folded precursor RNA into VGAM113 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM113 RNA is designated SEQ ID:448, and is provided hereinbelow with reference to the sequence listing part.

VGAM113 host target gene, herein designated VGAM HOST TARGET GENE, encodes a corresponding messenger RNA, VGAM113 host target RNA, herein designated VGAM HOST TARGET RNA. VGAM113 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM113 RNA, herein designated VGAM RNA, binds complementarily to one or more host target binding sites located in untranslated regions of VGAM113 host target RNA, herein designated VGAM HOST TARGET RNA. This complementary binding is due to the fact that the nucleotide sequence of VGAM113 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM113 RNA, herein designated VGAM RNA, may have a different number of host target binding sites in untranslated regions of a VGAM113 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM113 RNA, herein designated VGAM RNA, to host target binding sites on VGAM113 host target RNA, herein designated VGAM HOST TARGET RNA, such as BINDING SITE I, BINDING SITE II and BINDING SITE III, inhibits translation of VGAM113 host target RNA into VGAM113 host target protein, herein designated VGAM HOST TARGET PROTEIN. VGAM host target protein is therefore outlined by a broken line.

It is appreciated that VGAM113 host target gene, herein designated VGAM HOST TARGET GENE, in fact represents a plurality of VGAM113 host target genes. The mRNA of each one of this plurality of VGAM113 host target genes comprises one or more host target binding sites, each having a nucleotide sequence which is at least partly complementary to VGAM113 RNA, herein designated VGAM RNA, and which when bound by VGAM113 RNA causes inhibition of translation of respective one or more VGAM113 host target proteins.

It is further appreciated by one skilled in the art that the mode of translational inhibition illustrated by FIG. 1 with specific reference to translational inhibition exerted by VGAM113 gene, herein designated VGAM GENE, on one or more VGAM113 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM113 is inhibition of expression of host target genes, as part of a novel viral mechanism of attacking a host. Accordingly, utilities of VGAM113 include diagnosis, prevention and treatment of viral infection by Vaccinia Virus. Specific functions, and accordingly utilities, of VGAM113 correlate with, and may be deduced from, the identity of the host target genes which VGAM113 binds and inhibits, and the function of these host target genes, as elaborated hereinbelow.

Nucleotide sequences of the VGAM113 precursor RNA, herein designated VGAM PRECURSOR RNA, and of the ‘diced’ VGAM113 RNA, herein designated VGAM RNA, and a schematic representation of the secondary folding of VGAM113 folded precursor RNA, herein designated VGAM FOLDED PRECURSOR RNA, of VGAM113 are further described hereinbelow with reference to Table 1.

Nucleotide sequences of host target binding sites, such as BINDING SITE-I, BINDING SITE-II and BINDING SITE-III of FIG. 1, found on VGAM113 host target RNA, and schematic representation of the complementarity of each of these host target binding sites to VGAM113 RNA, herein designated VGAM RNA, are described hereinbelow with reference to Table 2.

As mentioned hereinabove with reference to FIG. 1, a function of VGAM113 gene, herein designated VGAM is inhibition of expression of VGAM113 target genes. It is appreciated that specific functions, and accordingly utilities, of VGAM113 correlate with, and may be deduced from, the identity of the target genes which VGAM113 binds and inhibits, and the function of these target genes, as elaborated hereinbelow.

Complement Component 3a Receptor 1 (C3AR1, Accession NM_{—}004054) is a VGAM113 host target gene. C3AR1 BINDING SITE is HOST TARGET binding site found in the 3′ untranslated region of mRNA encoded by C3AR1, corresponding to a HOST TARGET binding site such as BINDING SITE I, BINDING SITE II or BINDING SITE III. Table 2 illustrates the complementarity of the nucleotide sequences of C3AR1 BINDING SITE, designated SEQ ID: 1081, to the nucleotide sequence of VGAM113 RNA, herein designated VGAM RNA, also designated SEQ ID:448.

A function of VGAM113 is therefore inhibition of Complement Component 3a Receptor 1 (C3AR1, Accession NM_{—}004054). Accordingly, utilities of VGAM113 include diagnosis, prevention and treatment of diseases and clinical conditions associated with C3AR1. GABA(A) Receptors Associated Protein Like 3 (GABARAPL3, Accession NM_{—}032568) is another VGAM113 host target gene. GABARAPL3 BINDING SITE is HOST TARGET binding site found in the 3′ untranslated region of mRNA encoded by GABARAPL3, corresponding to a HOST TARGET binding site such as BINDING SITE I, BINDING SITE II or BINDING SITE III. Table 2 illustrates the complementarity of the nucleotide sequences of GABARAPL3 BINDING SITE, designated SEQ ID:2260, to the nucleotide sequence of VGAM113 RNA, herein designated VGAM RNA, also designated SEQ ID:448.

Another function of VGAM113 is therefore inhibition of GABA(A) Receptors Associated Protein Like 3 (GABARAPL3, Accession NM_{—}032568). Accordingly, utilities of VGAM113 include diagnosis, prevention and treatment of diseases and clinical conditions associated with GABARAPL3. Golgi Autoantigen, Golgin Sub family A, 2-like, Y-linked (GOLGA2LY, Accession XM_{—}034789) is another VGAM113 host target gene. GOLGA2LY BINDING SITE is HOST TARGET binding site found in the 5′ untranslated region of mRNA encoded by GOLGA2LY, corresponding to a HOST TARGET binding site such as BINDING SITE I, BINDING SITE II or BINDING SITE III. Table 2 illustrates the complementarity of the nucleotide sequences of GOLGA2LY BINDING SITE, designated SEQ ID:2684, to the nucleotide sequence of VGAM113 RNA, herein designated VGAM RNA, also designated SEQ ID:448.

Another function of VGAM113 is therefore inhibition of Golgi Autoantigen, Golgin Sub family A, 2-like, Y-linked (GOLGA2LY, Accession XM_{—}034789). Accordingly, utilities of VGAM113 include diagnosis, prevention and treatment of diseases and clinical conditions associated with GOLGA2LY. Paternally Expressed 10 (PEG10, Accession NM_{—}015068) is another VGAM113 host target gene. PEG10 BINDING SITE is HOST TARGET binding site found in the 3′ untranslated region of mRNA encoded by PEG10, corresponding to a HOST TARGET binding site such as BINDING SITE I, BINDING SITE II or BINDING SITE III. Table 2 illustrates the complementarity of the nucleotide sequences of PEG10 BINDING SITE, designated SEQ ID:1607, to the nucleotide sequence of VGAM113 RNA, herein designated VGAM RNA, also designated SEQ ID:448.

Another function of VGAM113 is therefore inhibition of Paternally Expressed 10 (PEG10, Accession NM_{—}015068). Accordingly, utilities of VGAM113 include diagnosis, prevention and treatment of diseases and clinical conditions associated with PEG10. Ring Finger Protein 13 (RNF13, Accession NM_{—}007282) is another VGAM113 host target gene. RNF13 BINDING SITE is HOST TARGET binding site found in the 3′ untranslated region of mRNA encoded by RNF13, corresponding to a HOST TARGET binding site such as BINDING SITE I, BINDING SITE II or BINDING SITE III. Table 2 illustrates the complementarity of the nucleotide sequences of RNF13 BINDING SITE, designated SEQ ID:1376, to the nucleotide sequence of VGAM113 RNA, herein designated VGAM RNA, also designated SEQ ID:448.

Another function of VGAM113 is therefore inhibition of Ring Finger Protein 13 (RNF13, Accession NM_{—}007282). Accordingly, utilities of VGAM113 include diagnosis, prevention and treatment of diseases and clinical conditions associated with RNF13. LOC143692 (Accession XM_{—}084601) is another VGAM113 host target gene. LOC143692 BINDING SITE is HOST TARGET binding site found in the 5′ untranslated region of mRNA encoded by LOC143692, corresponding to a HOST TARGET binding site such as BINDING SITE I, BINDING SITE II or BINDING SITE III. Table 2 illustrates the complementarity of the nucleotide sequences of LOC143692 BINDING SITE, designated SEQ ID:3060, to the nucleotide sequence of VGAM113 RNA, herein designated VGAM RNA, also designated SEQ ID:448.

Another function of VGAM113 is therefore inhibition of LOC143692 (Accession XM_{—}084601). Accordingly, utilities of VGAM113 include diagnosis, prevention and treatment of diseases and clinical conditions associated with LOC143692. LOC151201 (Accession XM_{—}098021) is another VGAM113 host target gene. LOC151201 BINDING SITE is HOST TARGET binding site found in the 5′ untranslated region of mRNA encoded by LOC151201, corresponding to a HOST TARGET binding site such as BINDING SITE I, BINDING SITE II or BINDING SITE III. Table 2 illustrates the complementarity of the nucleotide sequences of LOC151201 BINDING SITE, designated SEQ ID:3342, to the nucleotide sequence of VGAM113 RNA, herein designated VGAM RNA, also designated SEQ ID:448.

Another function of VGAM113 is therefore inhibition of LOC151201 (Accession XM_{—}098021). Accordingly, utilities of VGAM113 include diagnosis, prevention and treatment of diseases and clinical conditions associated with LOC151201. LOC257431 (Accession XM_{—}088764) is another VGAM113 host target gene. LOC257431 BINDING SITE is HOST TARGET binding site found in the 5′ untranslated region of mRNA encoded by LOC257431, corresponding to a HOST TARGET binding site such as BINDING SITE I, BINDING SITE II or BINDING SITE III. Table 2 illustrates the complementarity of the nucleotide sequences of LOC257431 BINDING SITE, designated SEQ ID:3231, to the nucleotide sequence of VGAM113 RNA, herein designated VGAM RNA, also designated SEQ ID:448.

Another function of VGAM113 is therefore inhibition of LOC257431 (Accession XM_{—}088764). Accordingly, utilities of VGAM113 include diagnosis, prevention and treatment of diseases and clinical conditions associated with LOC257431. LOC93259 (Accession XM_{—}050105) is another VGAM113 host target gene. LOC93259 BINDING SITE is HOST TARGET binding site found in the 5′ untranslated region of mRNA encoded by LOC93259, corresponding to a HOST TARGET binding site such as BINDING SITE I, BINDING SITE II or BINDING SITE III. Table 2 illustrates the complementarity of the nucleotide sequences of LOC93259 BINDING SITE, designated SEQ ID:2927, to the nucleotide sequence of VGAM113 RNA, herein designated VGAM RNA, also designated SEQ ID:448.

Another function of VGAM113 is therefore inhibition of LOC93259 (Accession XM_{—}050105). Accordingly, utilities of VGAM113 include diagnosis, prevention and treatment of diseases and clinical conditions associated with LOC93259. FIG. 1 further provides a conceptual description of a novel bioinformatically detected viral gene of the present invention, referred to here as Viral Genomic Address Messenger 114 (VGAM114) viral gene, which modulates expression of respective host target genes thereof, the function and utility of which host target genes is known in the art.

VGAM114 is a novel bioinformatically detected regulatory, non protein coding, viral micro RNA (miRNA) gene. The method by which VGAM114 was detected is described hereinabove with reference to FIGS. 1-8.

VGAM114 gene, herein designated VGAM GENE, is a viral gene contained in the genome of Vaccinia Virus. VGAM114 host target gene, herein designated VGAM HOST TARGET GENE, is a human gene contained in the human genome.

VGAM114 gene encodes a VGAM114 precursor RNA, herein designated VGAM PRECURSOR RNA. Similar to other miRNA genes, and unlike most ordinary genes, VGAM114 precursor RNA does not encode a protein. A nucleotide sequence identical or highly similar to the nucleotide sequence of VGAM114 precursor RNA is designated SEQ ID:100, and is provided hereinbelow with reference to the sequence listing part. Nucleotide sequence SEQ ID: 100 is located at position 49722 relative to the genome of Vaccinia Virus.

VGAM114 precursor RNA folds onto itself, forming VGAM114 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM114 folded precursor RNA into VGAM114 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM114 RNA is designated SEQ ID:449, and is provided hereinbelow with reference to the sequence listing part.

VGAM114 host target gene, herein designated VGAM HOST TARGET GENE, encodes a corresponding messenger RNA, VGAM114 host target RNA, herein designated VGAM HOST TARGET RNA. VGAM114 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM114 RNA, herein designated VGAM RNA, binds complementarily to one or more host target binding sites located in untranslated regions of VGAM114 host target RNA, herein designated VGAM HOST TARGET RNA. This complementary binding is due to the fact that the nucleotide sequence of VGAM114 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM114 RNA, herein designated VGAM RNA, may have a different number of host target binding sites in untranslated regions of a VGAM114 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM114 RNA, herein designated VGAM RNA, to host target binding sites on VGAM114 host target RNA, herein designated VGAM HOST TARGET RNA, such as BINDING SITE I, BINDING SITE II and BINDING SITE III, inhibits translation of VGAM114 host target RNA into VGAM114 host target protein, herein designated VGAM HOST TARGET PROTEIN. VGAM host target protein is therefore outlined by a broken line.

It is appreciated that VGAM114 host target gene, herein designated VGAM HOST TARGET GENE, in fact represents a plurality of VGAM114 host target genes. The mRNA of each one of this plurality of VGAM114 host target genes comprises one or more host target binding sites, each having a nucleotide sequence which is at least partly complementary to VGAM114 RNA, herein designated VGAM RNA, and which when bound by VGAM114 RNA causes inhibition of translation of respective one or more VGAM114 host target proteins.

It is further appreciated by one skilled in the art that the mode of translational inhibition illustrated by FIG. 1 with specific reference to translational inhibition exerted by VGAM114 gene, herein designated VGAM GENE, on one or more VGAM114 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM114 is inhibition of expression of host target genes, as part of a novel viral mechanism of attacking a host. Accordingly, utilities of VGAM114 include diagnosis, prevention and treatment of viral infection by Vaccinia Virus. Specific functions, and accordingly utilities, of VGAM114 correlate with, and may be deduced from, the identity of the host target genes which VGAM114 binds and inhibits, and the function of these host target genes, as elaborated hereinbelow.

Nucleotide sequences of the VGAM114 precursor RNA, herein designated VGAM PRECURSOR RNA, and of the ‘diced’ VGAM114 RNA, herein designated VGAM RNA, and a schematic representation of the secondary folding of VGAM114 folded precursor RNA, herein designated VGAM FOLDED PRECURSOR RNA, of VGAM114 are further described hereinbelow with reference to Table 1.

Nucleotide sequences of host target binding sites, such as BINDING SITE-I, BINDING SITE-II and BINDING SITE-III of FIG. 1, found on VGAM114 host target RNA, and schematic representation of the complementarity of each of these host target binding sites to VGAM114 RNA, herein designated VGAM RNA, are described hereinbelow with reference to Table 2.

As mentioned hereinabove with reference to FIG. 1, a function of VGAM114 gene, herein designated VGAM is inhibition of expression of VGAM114 target genes. It is appreciated that specific functions, and accordingly utilities, of VGAM114 correlate with, and may be deduced from, the identity of the target genes which VGAM114 binds and inhibits, and the function of these target genes, as elaborated hereinbelow.

SAM Domain and HD Domain 1 (SAMHD1, Accession XM_{—}028704) is a VGAM114 host target gene. SAMHD1 BINDING SITE is HOST TARGET binding site found in the 3′ untranslated region of mRNA encoded by SAMHD1, corresponding to a HOST TARGET binding site such as BINDING SITE I, BINDING SITE II or BINDING SITE III. Table 2 illustrates the complementarity of the nucleotide sequences of SAMHD1 BINDING SITE, designated SEQ ID:2594, to the nucleotide sequence of VGAM114 RNA, herein designated VGAM RNA, also designated SEQ ID:449.

A function of VGAM114 is therefore inhibition of SAM Domain and HD Domain 1 (SAMHD1, Accession XM_{—}028704). Accordingly, utilities of VGAM114 include diagnosis, prevention and treatment of diseases and clinical conditions associated with SAMHD1. LOC150864 (Accession XM_{—}087026) is another VGAM114 host target gene. LOC150864 BINDING SITE is HOST TARGET binding site found in the 5′ untranslated region of mRNA encoded by LOC150864, corresponding to a HOST TARGET binding site such as BINDING SITE I, BINDING SITE II or BINDING SITE III. Table 2 illustrates the complementarity of the nucleotide sequences of LOC150864 BINDING SITE, designated SEQ ID:3150, to the nucleotide sequence of VGAM114 RNA, herein designated VGAM RNA, also designated SEQ ID:449.

Another function of VGAM114 is therefore inhibition of LOC150864 (Accession XM_{—}087026). Accordingly, utilities of VGAM114 include diagnosis, prevention and treatment of diseases and clinical conditions associated with LOC150864. LOC257358 (Accession XM_{—}173138) is another VGAM114 host target gene. LOC257358 BINDING SITE is HOST TARGET binding site found in the 3′ untranslated region of mRNA encoded by LOC257358, corresponding to a HOST TARGET binding site such as BINDING SITE I, BINDING SITE II or BINDING SITE III. Table 2 illustrates the complementarity of the nucleotide sequences of LOC257358 BINDING SITE, designated SEQ ID:3731, to the nucleotide sequence of VGAM114 RNA, herein designated VGAM RNA, also designated SEQ ID:449.

Another function of VGAM114 is therefore inhibition of LOC257358 (Accession XM_{—}173138). Accordingly, utilities of VGAM114 include diagnosis, prevention and treatment of diseases and clinical conditions associated with LOC257358. LOC90288 (Accession XM_{—}030669) is another VGAM114 host target gene. LOC90288 BINDING SITE is HOST TARGET binding site found in the 3′ untranslated region of mRNA encoded by LOC90288, corresponding to a HOST TARGET binding site such as BINDING SITE I, BINDING SITE II or BINDING SITE III. Table 2 illustrates the complementarity of the nucleotide sequences of LOC90288 BINDING SITE, designated SEQ ID:2621, to the nucleotide sequence of VGAM114 RNA, herein designated VGAM RNA, also designated SEQ ID:449.

Another function of VGAM114 is therefore inhibition of LOC90288 (Accession XM_{—}030669). Accordingly, utilities of VGAM114 include diagnosis, prevention and treatment of diseases and clinical conditions associated with LOC90288. FIG. 1 further provides a conceptual description of a novel bioinformatically detected viral gene of the present invention, referred to here as Viral Genomic Address Messenger 115 (VGAM115) viral gene, which modulates expression of respective host target genes thereof, the function and utility of which host target genes is known in the art.

VGAM115 is a novel bioinformatically detected regulatory, non protein coding, viral micro RNA (miRNA) gene. The method by which VGAM115 was detected is described hereinabove with reference to FIGS. 1-8.

VGAM115 gene, herein designated VGAM GENE, is a viral gene contained in the genome of Vaccinia Virus. VGAM115 host target gene, herein designated VGAM HOST TARGET GENE, is a human gene contained in the human genome.

VGAM115 gene encodes a VGAM115 precursor RNA, herein designated VGAM PRECURSOR RNA. Similar to other miRNA genes, and unlike most ordinary genes, VGAM115 precursor RNA does not encode a protein. A nucleotide sequence identical or highly similar to the nucleotide sequence of VGAM115 precursor RNA is designated SEQ ID:101, and is provided hereinbelow with reference to the sequence listing part. Nucleotide sequence SEQ ID: 101 is located at position 49784 relative to the genome of Vaccinia Virus.

VGAM115 precursor RNA folds onto itself, forming VGAM115 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM115 folded precursor RNA into VGAM115 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM115 RNA is designated SEQ ID:450, and is provided hereinbelow with reference to the sequence listing part.

VGAM115 host target gene, herein designated VGAM HOST TARGET GENE, encodes a corresponding messenger RNA, VGAM115 host target RNA, herein designated VGAM HOST TARGET RNA. VGAM115 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM115 RNA, herein designated VGAM RNA, binds complementarily to one or more host target binding sites located in untranslated regions of VGAM115 host target RNA, herein designated VGAM HOST TARGET RNA. This complementary binding is due to the fact that the nucleotide sequence of VGAM115 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM115 RNA, herein designated VGAM RNA, may have a different number of host target binding sites in untranslated regions of a VGAM115 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM115 RNA, herein designated VGAM RNA, to host target binding sites on VGAM115 host target RNA, herein designated VGAM HOST TARGET RNA, such as BINDING SITE I, BINDING SITE II and BINDING SITE III, inhibits translation of VGAM115 host target RNA into VGAM115 host target protein, herein designated VGAM HOST TARGET PROTEIN. VGAM host target protein is therefore outlined by a broken line.

It is appreciated that VGAM115 host target gene, herein designated VGAM HOST TARGET GENE, in fact represents a plurality of VGAM115 host target genes. The mRNA of each one of this plurality of VGAM115 host target genes comprises one or more host target binding sites, each having a nucleotide sequence which is at least partly complementary to VGAM115 RNA, herein designated VGAM RNA, and which when bound by VGAM115 RNA causes inhibition of translation of respective one or more VGAM115 host target proteins.

It is further appreciated by one skilled in the art that the mode of translational inhibition illustrated by FIG. 1 with specific reference to translational inhibition exerted by VGAM115 gene, herein designated VGAM GENE, on one or more VGAM115 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM115 is inhibition of expression of host target genes, as part of a novel viral mechanism of attacking a host. Accordingly, utilities of VGAM115 include diagnosis, prevention and treatment of viral infection by Vaccinia Virus. Specific functions, and accordingly utilities, of VGAM115 correlate with, and may be deduced from, the identity of the host target genes which VGAM115 binds and inhibits, and the function of these host target genes, as elaborated hereinbelow.

Nucleotide sequences of the VGAM115 precursor RNA, herein designated VGAM PRECURSOR RNA, and of the ‘diced’ VGAM115 RNA, herein designated VGAM RNA, and a schematic representation of the secondary folding of VGAM115 folded precursor RNA, herein designated VGAM FOLDED PRECURSOR RNA, of VGAM115 are further described hereinbelow with reference to Table 1.

Nucleotide sequences of host target binding sites, such as BINDING SITE-I, BINDING SITE-II and BINDING SITE-III of FIG. 1, found on VGAM115 host target RNA, and schematic representation of the complementarity of each of these host target binding sites to VGAM115 RNA, herein designated VGAM RNA, are described hereinbelow with reference to Table 2.

As mentioned hereinabove with reference to FIG. 1, a function of VGAM115 gene, herein designated VGAM is inhibition of expression of VGAM115 target genes. It is appreciated that specific functions, and accordingly utilities, of VGAM115 correlate with, and may be deduced from, the identity of the target genes which VGAM115 binds and inhibits, and the function of these target genes, as elaborated hereinbelow.

Glutamate Decarboxylase 1 (brain, 67 kDa) (GAD1, Accession NM_{—}000817) is a VGAM115 host target gene. GAD1 BINDING SITE is HOST TARGET binding site found in the 3′ untranslated region of mRNA encoded by GAD1, corresponding to a HOST TARGET binding site such as BINDING SITE I, BINDING SITE II or BINDING SITE III. Table 2 illustrates the complementarity of the nucleotide sequences of GAD1 BINDING SITE, designated SEQ ID:777, to the nucleotide sequence of VGAM115 RNA, herein designated VGAM RNA, also designated SEQ ID:450.

A function of VGAM115 is therefore inhibition of Glutamate Decarboxylase 1 (brain, 67 kDa) (GAD1, Accession NM_{—}000817), a gene which catalyzes the conversion of glutamic acid to gamma-aminobutyric acid. Accordingly, utilities of VGAM115 include diagnosis, prevention and treatment of diseases and clinical conditions associated with GAD1. The function of GAD1 has been established by previous studies. Using a genomic probe from a human fetal brain library, Sparkes et al. (1987) probed the DNA of a mouse/human somatic cell hybrid panel and assigned the GAD gene to human chromosome 2. Kelly et al. (1992) confirmed the assignment of GAD1 to chromosome 2, using PCR to amplify specifically the human sequence in rodent/human somatic cell hybrid DNAs. They also reported the full nucleotide sequence of the cDNA and the deduced amino acid sequence. Bu et al. (1992) mapped the GAD1 gene to 2q31 by in situ hybridization of fluorescently labeled GAD probes to human chromosomes. They demonstrated that the GAD1 gene encodes a polypeptide of 67,000 molecular weight, with 594 amino acid residues. The GAD2 gene (OMIM Ref. No. 138275), located on 10p11.23, encodes a polypeptide of 65,000 molecular weight (GAD65), with 585 amino acid residues. Brilliant et al. (1990) showed by Southern analysis of mouse-hamster hybrid cells and by interspecific backcrosses and recombinant inbred strains that the mouse equivalent (Gad1) is located on chromosome 2 and that an apparent pseudogene is located on mouse chromosome 10. The mouse Gad1 locus is part of a conserved homology between mouse chromosome 2 and human 2q. By in situ hybridization, Edelhoff et al. (1993) also assigned GAD1 to human 2q31 and to mouse chromosome 2D in a known region of conservation between human and mouse. Animal model experiments lend further support to the function of GAD1. The remaining exon-intron boundaries occur at identical positions in the 2 cDNAs, suggesting that they derive from a common ancestral GAD gene. In addition to its role as an inhibitory neurotransmitter, GABA is presumed to be involved in the development and plasticity of the nervous system. GABA is synthesized by glutamic acid decarboxylase, but the respective roles of its 2 isoforms, GAD65 and GAD67, had not been determined. Asada et al. (1996, 1997) undertook the selective elimination of each GAD isoform by gene targeting to clarify this issue. Asada et al. (1996) found that GAD65 −/− mice showed no change in brain GABA content or animal behavior, except for a slight increase in susceptibility to seizures. Asada et al. (1997) produced GAD67 −/− mice. These mice were born at the expected frequency but died of severe cleft palate during the first morning after birth. GAD activities and GABA contents were reduced to 20 and 7%, respectively, in the cerebral cortex of the newborn GAD67 −/− mice. Their brains, however, did not show any discernible defects. Previous pharmacologic and genetic investigations suggested the involvement of GABA in palate formation, but this was the first demonstration of a role for GAD67-derived GABA in the development of non-neural tissue. Independently, Condie et al. (1997) found defects in the formation of the palate in mice with a targeted mutation in the gene encoding GAD67. Previous observations had suggested a role of GABA in palate development. Analysis of mice with mutations in the beta-3 gamma-GABA receptor (GABRB3; 137192) had demonstrated that these mutations are associated with cleft secondary palate in mice. The phenotype in the GABRB3 mutants showed that this gene is somehow involved in palate development but did not demonstrate that GABA is the ligand involved in this particular function. The results of Condie et al. (1997), demonstrating a similar phenotype between the receptor and ligand mutations, demonstrated a role for GABA signaling in normal palate development.

It is appreciated that the abovementioned animal model for GAD1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Asada, H.; Kawamura, Y.; Maruyama, K.; Kume, H.; Ding, R.-G.; Ji, F. Y.; Kanbara, N.; Kuzume, H.; Sanbo, M.; Yagi, T.; Obata, K.: Mice lacking the 65 kDa isoform of glutamic acid decarboxylase (GAD65) maintain normal levels of GAD67 and GABA in their brains but are susceptible to seizures. Biochem. Biophys. Res. Commun. 229: 891-895, 1996.; and
Scriver, C. R.; Hutchison, J. H.: The vitamin B6 deficiency syndrome in human infancy: biochemical and clinical observations. Pediatrics 31: 240-250, 1963.

Further studies establishing the function and utilities of GAD1 are found in John Hopkins OMIM database record ID 605363, and in sited publications numbered 2764-1540, 2765-1542, 2766-1545, 309-31 and 1546 listed in the bibliography section hereinbelow, which are also hereby incorporated by reference. Kelch-like 2, Mayven (Drosophila) (KLHL2, Accession NM_{—}007246) is another VGAM115 host target gene. KLHL2 BINDING SITE is HOST TARGET binding site found in the 3′ untranslated region of mRNA encoded by KLHL2, corresponding to a HOST TARGET binding site such as BINDING SITE I, BINDING SITE II or BINDING SITE III. Table 2 illustrates the complementarity of the nucleotide sequences of KLHL2 BINDING SITE, designated SEQ ID:1371, to the nucleotide sequence of VGAM115 RNA, herein designated VGAM RNA, also designated SEQ ID:450.

Another function of VGAM115 is therefore inhibition of Kelch-like 2, Mayven (Drosophila) (KLHL2, Accession NM_{—}007246). Accordingly, utilities of VGAM115 include diagnosis, prevention and treatment of diseases and clinical conditions associated with KLHL2. KIAA0276 (Accession XM_{—}048199) is another VGAM115 host target gene. KIAA0276 BINDING SITE is HOST TARGET binding site found in the 3′ untranslated region of mRNA encoded by KIAA0276, corresponding to a HOST TARGET binding site such as BINDING SITE I, BINDING SITE II or BINDING SITE III. Table 2 illustrates the complementarity of the nucleotide sequences of KIAA0276 BINDING SITE, designated SEQ ID:2904, to the nucleotide sequence of VGAM115 RNA, herein designated VGAM RNA, also designated SEQ ID:450.

Another function of VGAM115 is therefore inhibition of KIAA0276 (Accession XM_{—}048199). Accordingly, utilities of VGAM115 include diagnosis, prevention and treatment of diseases and clinical conditions associated with KIAA0276. LOC130814 (Accession XM_{—}059471) is another VGAM115 host target gene. LOC130814 BINDING SITE is HOST TARGET binding site found in the 3′ untranslated region of mRNA encoded by LOC130814, corresponding to a HOST TARGET binding site such as BINDING SITE I, BINDING SITE II or BINDING SITE III. Table 2 illustrates the complementarity of the nucleotide sequences of LOC130814 BINDING SITE, designated SEQ ID:3012, to the nucleotide sequence of VGAM115 RNA, herein designated VGAM RNA, also designated SEQ ID:450.

Another function of VGAM115 is therefore inhibition of LOC130814 (Accession XM_{—}059471). Accordingly, utilities of VGAM115 include diagnosis, prevention and treatment of diseases and clinical conditions associated with LOC130814. FIG. 1 further provides a conceptual description of a novel bioinformatically detected viral gene of the present invention, referred to here as Viral Genomic Address Messenger 116 (VGAM116) viral gene, which modulates expression of respective host target genes thereof, the function and utility of which host target genes is known in the art.

VGAM116 is a novel bioinformatically detected regulatory, non protein coding, viral micro RNA (miRNA) gene. The method by which VGAM116 was detected is described hereinabove with reference to FIGS. 1-8.

VGAM116 gene, herein designated VGAM GENE, is a viral gene contained in the genome of Vaccinia Virus. VGAM116 host target gene, herein designated VGAM HOST TARGET GENE, is a human gene contained in the human genome.

VGAM116 gene encodes a VGAM116 precursor RNA, herein designated VGAM PRECURSOR RNA. Similar to other miRNA genes, and unlike most ordinary genes, VGAM116 precursor RNA does not encode a protein. A nucleotide sequence identical or highly similar to the nucleotide sequence of VGAM116 precursor RNA is designated SEQ ID:102, and is provided hereinbelow with reference to the sequence listing part. Nucleotide sequence SEQ ID: 102 is located at position 48815 relative to the genome of Vaccinia Virus.

VGAM116 precursor RNA folds onto itself, forming VGAM116 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM116 folded precursor RNA into VGAM116 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM116 RNA is designated SEQ ID:451, and is provided hereinbelow with reference to the sequence listing part.

VGAM116 host target gene, herein designated VGAM HOST TARGET GENE, encodes a corresponding messenger RNA, VGAM116 host target RNA, herein designated VGAM HOST TARGET RNA. VGAM116 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM116 RNA, herein designated VGAM RNA, binds complementarily to one or more host target binding sites located in untranslated regions of VGAM116 host target RNA, herein designated VGAM HOST TARGET RNA. This complementary binding is due to the fact that the nucleotide sequence of VGAM116 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM116 RNA, herein designated VGAM RNA, may have a different number of host target binding sites in untranslated regions of a VGAM116 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM116 RNA, herein designated VGAM RNA, to host target binding sites on VGAM116 host target RNA, herein designated VGAM HOST TARGET RNA, such as BINDING SITE I, BINDING SITE II and BINDING SITE III, inhibits translation of VGAM116 host target RNA into VGAM116 host target protein, herein designated VGAM HOST TARGET PROTEIN. VGAM host target protein is therefore outlined by a broken line.

It is appreciated that VGAM116 host target gene, herein designated VGAM HOST TARGET GENE, in fact represents a plurality of VGAM116 host target genes. The mRNA of each one of this plurality of VGAM116 host target genes comprises one or more host target binding sites, each having a nucleotide sequence which is at least partly complementary to VGAM116 RNA, herein designated VGAM RNA, and which when bound by VGAM116 RNA causes inhibition of translation of respective one or more VGAM116 host target proteins.

It is further appreciated by one skilled in the art that the mode of translational inhibition illustrated by FIG. 1 with specific reference to translational inhibition exerted by VGAM116 gene, herein designated VGAM GENE, on one or more VGAM116 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM116 is inhibition of expression of host target genes, as part of a novel viral mechanism of attacking a host. Accordingly, utilities of VGAM116 include diagnosis, prevention and treatment of viral infection by Vaccinia Virus. Specific functions, and accordingly utilities, of VGAM116 correlate with, and may be deduced from, the identity of the host target genes which VGAM116 binds and inhibits, and the function of these host target genes, as elaborated hereinbelow.

Nucleotide sequences of the VGAM116 precursor RNA, herein designated VGAM PRECURSOR RNA, and of the ‘diced’ VGAM116 RNA, herein designated VGAM RNA, and a schematic representation of the secondary folding of VGAM116 folded precursor RNA, herein designated VGAM FOLDED PRECURSOR RNA, of VGAM116 are further described hereinbelow with reference to Table 1.

Nucleotide sequences of host target binding sites, such as BINDING SITE-I, BINDING SITE-II and BINDING SITE-III of FIG. 1, found on VGAM116 host target RNA, and schematic representation of the complementarity of each of these host target binding sites to VGAM116 RNA, herein designated VGAM RNA, are described hereinbelow with reference to Table 2.

As mentioned hereinabove with reference to FIG. 1, a function of VGAM116 gene, herein designated VGAM is inhibition of expression of VGAM116 target genes. It is appreciated that specific functions, and accordingly utilities, of VGAM116 correlate with, and may be deduced from, the identity of the target genes which VGAM116 binds and inhibits, and the function of these target genes, as elaborated hereinbelow.

Transforming Growth Factor, Beta Receptor II (70/80 kDa) (TGFBR2, Accession NM_{—}003242) is a VGAM116 host target gene. TGFBR2 BINDING SITE is HOST TARGET binding site found in the 3′ untranslated region of mRNA encoded by TGFBR2, corresponding to a HOST TARGET binding site such as BINDING SITE I, BINDING SITE II or BINDING SITE III. Table 2 illustrates the complementarity of the nucleotide sequences of TGFBR2 BINDING SITE, designated SEQ ID: 1002, to the nucleotide sequence of VGAM116 RNA, herein designated VGAM RNA, also designated SEQ ID:451.

A function of VGAM116 is therefore inhibition of Transforming Growth Factor, Beta Receptor II (70/80 kDa) (TGFBR2, Accession NM_{—}003242). Accordingly, utilities of VGAM116 include diagnosis, prevention and treatment of diseases and clinical conditions associated with TGFBR2. KIAA1223 (Accession XM_{—}048747) is another VGAM116 host target gene. KIAA1223 BINDING SITE is HOST TARGET binding site found in the 3′ untranslated region of mRNA encoded by KIAA1223, corresponding to a HOST TARGET binding site such as BINDING SITE I, BINDING SITE II or BINDING SITE III. Table 2 illustrates the complementarity of the nucleotide sequences of KIAA1223 BINDING SITE, designated SEQ ID:2908, to the nucleotide sequence of VGAM116 RNA, herein designated VGAM RNA, also designated SEQ ID:451.

Another function of VGAM116 is therefore inhibition of KIAA1223 (Accession XM_{—}048747). Accordingly, utilities of VGAM116 include diagnosis, prevention and treatment of diseases and clinical conditions associated with KIAA1223. PRO2859 (Accession NM_{—}018543) is another VGAM116 host target gene. PRO2859 BINDING SITE is HOST TARGET binding site found in the 5′ untranslated region of mRNA encoded by PRO2859, corresponding to a HOST TARGET binding site such as BINDING SITE I, BINDING SITE II or BINDING SITE III. Table 2 illustrates the complementarity of the nucleotide sequences of PRO2859 BINDING SITE, designated SEQ ID:1840, to the nucleotide sequence of VGAM116 RNA, herein designated VGAM RNA, also designated SEQ ID:451.

Another function of VGAM116 is therefore inhibition of PRO2859 (Accession NM_{—}018543). Accordingly, utilities of VGAM116 include diagnosis, prevention and treatment of diseases and clinical conditions associated with PRO2859. PC4 and SFRS1 Interacting Protein 2 (PSIP2, Accession NM_{—}033222) is another VGAM116 host target gene. PSIP2 BINDING SITE is HOST TARGET binding site found in the 3′ untranslated region of mRNA encoded by PSIP2, corresponding to a HOST TARGET binding site such as BINDING SITE I, BINDING SITE II or BINDING SITE III. Table 2 illustrates the complementarity of the nucleotide sequences of PSIP2 BINDING SITE, designated SEQ ID:2324, to the nucleotide sequence of VGAM116 RNA, herein designated VGAM RNA, also designated SEQ ID:451.

Another function of VGAM116 is therefore inhibition of PC4 and SFRS1 Interacting Protein 2 (PSIP2, Accession NM_{—}033222). Accordingly, utilities of VGAM116 include diagnosis, prevention and treatment of diseases and clinical conditions associated with PSIP2. LOC152185 (Accession NM_{—}144718) is another VGAM116 host target gene. LOC152185 BINDING SITE is HOST TARGET binding site found in the 3′ untranslated region of mRNA encoded by LOC152185, corresponding to a HOST TARGET binding site such as BINDING SITE I, BINDING SITE II or BINDING SITE III. Table 2 illustrates the complementarity of the nucleotide sequences of LOC152185 BINDING SITE, designated SEQ ID:2497, to the nucleotide sequence of VGAM116 RNA, herein designated VGAM RNA, also designated SEQ ID:451.

Another function of VGAM116 is therefore inhibition of LOC152185 (Accession NM_{—}144718). Accordingly, utilities of VGAM116 include diagnosis, prevention and treatment of diseases and clinical conditions associated with LOC152185. LOC89953 (Accession NM_{—}138343) is another VGAM116 host target gene. LOC89953 BINDING SITE is HOST TARGET binding site found in the 3′ untranslated region of mRNA encoded by LOC89953, corresponding to a HOST TARGET binding site such as BINDING SITE I, BINDING SITE II or BINDING SITE III. Table 2 illustrates the complementarity of the nucleotide sequences of LOC89953 BINDING SITE, designated SEQ ID:2437, to the nucleotide sequence of VGAM116 RNA, herein designated VGAM RNA, also designated SEQ ID:451.

Another function of VGAM116 is therefore inhibition of LOC89953 (Accession NM_{—}138343). Accordingly, utilities of VGAM116 include diagnosis, prevention and treatment of diseases and clinical conditions associated with LOC89953. LOC90509 (Accession XM_{—}032209) is another VGAM116 host target gene. LOC90509 BINDING SITE is HOST TARGET binding site found in the 5′ untranslated region of mRNA encoded by LOC90509, corresponding to a HOST TARGET binding site such as BINDING SITE I, BINDING SITE II or BINDING SITE III. Table 2 illustrates the complementarity of the nucleotide sequences of LOC90509 BINDING SITE, designated SEQ ID:2649, to the nucleotide sequence of VGAM116 RNA, herein designated VGAM RNA, also designated SEQ ID:451.

Another function of VGAM116 is therefore inhibition of LOC90509 (Accession XM_{—}032209). Accordingly, utilities of VGAM116 include diagnosis, prevention and treatment of diseases and clinical conditions associated with LOC90509. FIG. 1 further provides a conceptual description of a novel bioinformatically detected viral gene of the present invention, referred to here as Viral Genomic Address Messenger 117 (VGAM117) viral gene, which modulates expression of respective host target genes thereof, the function and utility of which host target genes is known in the art.

VGAM117 is a novel bioinformatically detected regulatory, non protein coding, viral micro RNA (miRNA) gene. The method by which VGAM117 was detected is described hereinabove with reference to FIGS. 1-8.

VGAM117 gene, herein designated VGAM GENE, is a viral gene contained in the genome of Vaccinia Virus. VGAM117 host target gene, herein designated VGAM HOST TARGET GENE, is a human gene contained in the human genome.

VGAM117 gene encodes a VGAM117 precursor RNA, herein designated VGAM PRECURSOR RNA. Similar to other miRNA genes, and unlike most ordinary genes, VGAM117 precursor RNA does not encode a protein. A nucleotide sequence identical or highly similar to the nucleotide sequence of VGAM117 precursor RNA is designated SEQ ID:103, and is provided hereinbelow with reference to the sequence listing part. Nucleotide sequence SEQ ID: 103 is located at position 51120 relative to the genome of Vaccinia Virus.

VGAM117 precursor RNA folds onto itself, forming VGAM117 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM117 folded precursor RNA into VGAM117 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM117 RNA is designated SEQ ID:452, and is provided hereinbelow with reference to the sequence listing part.

VGAM117 host target gene, herein designated VGAM HOST TARGET GENE, encodes a corresponding messenger RNA, VGAM117 host target RNA, herein designated VGAM HOST TARGET RNA. VGAM117 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM117 RNA, herein designated VGAM RNA, binds complementarily to one or more host target binding sites located in untranslated regions of VGAM117 host target RNA, herein designated VGAM HOST TARGET RNA. This complementary binding is due to the fact that the nucleotide sequence of VGAM117 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM117 RNA, herein designated VGAM RNA, may have a different number of host target binding sites in untranslated regions of a VGAM117 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM117 RNA, herein designated VGAM RNA, to host target binding sites on VGAM117 host target RNA, herein designated VGAM HOST TARGET RNA, such as BINDING SITE I, BINDING SITE II and BINDING SITE III, inhibits translation of VGAM117 host target RNA into VGAM117 host target protein, herein designated VGAM HOST TARGET PROTEIN. VGAM host target protein is therefore outlined by a broken line.

It is appreciated that VGAM117 host target gene, herein designated VGAM HOST TARGET GENE, in fact represents a plurality of VGAM117 host target genes. The mRNA of each one of this plurality of VGAM117 host target genes comprises one or more host target binding sites, each having a nucleotide sequence which is at least partly complementary to VGAM117 RNA, herein designated VGAM RNA, and which when bound by VGAM117 RNA causes inhibition of translation of respective one or more VGAM117 host target proteins.

It is further appreciated by one skilled in the art that the mode of translational inhibition illustrated by FIG. 1 with specific reference to translational inhibition exerted by VGAM117 gene, herein designated VGAM GENE, on one or more VGAM117 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM117 is inhibition of expression of host target genes, as part of a novel viral mechanism of attacking a host. Accordingly, utilities of VGAM117 include diagnosis, prevention and treatment of viral infection by Vaccinia Virus. Specific functions, and accordingly utilities, of VGAM117 correlate with, and may be deduced from, the identity of the host target genes which VGAM117 binds and inhibits, and the function of these host target genes, as elaborated hereinbelow.

Nucleotide sequences of the VGAM117 precursor RNA, herein designated VGAM PRECURSOR RNA, and of the ‘diced’ VGAM117 RNA, herein designated VGAM RNA, and a schematic representation of the secondary folding of VGAM117 folded precursor RNA, herein designated VGAM FOLDED PRECURSOR RNA, of VGAM117 are further described hereinbelow with reference to Table 1.

Nucleotide sequences of host target binding sites, such as BINDING SITE-I, BINDING SITE-II and BINDING SITE-III of FIG. 1, found on VGAM117 host target RNA, and schematic representation of the complementarity of each of these host target binding sites to VGAM117 RNA, herein designated VGAM RNA, are described hereinbelow with reference to Table 2.

As mentioned hereinabove with reference to FIG. 1, a function of VGAM117 gene, herein designated VGAM is inhibition of expression of VGAM117 target genes. It is appreciated that specific functions, and accordingly utilities, of VGAM117 correlate with, and may be deduced from, the identity of the target genes which VGAM117 binds and inhibits, and the function of these target genes, as elaborated hereinbelow.

3′(2′), 5′-bisphosphate Nucleotidase 1 (BPNT1, Accession XM_{—}035738) is a VGAM117 host target gene. BPNT1 BINDING SITE is HOST TARGET binding site found in the 3′ untranslated region of mRNA encoded by BPNT1, corresponding to a HOST TARGET binding site such as BINDING SITE I, BINDING SITE II or BINDING SITE III. Table 2 illustrates the complementarity of the nucleotide sequences of BPNT1 BINDING SITE, designated SEQ ID:2702, to the nucleotide sequence of VGAM117 RNA, herein designated VGAM RNA, also designated SEQ ID:452.

A function of VGAM117 is therefore inhibition of 3′(2′), 5′-bisphosphate Nucleotidase 1 (BPNT1, Accession XM_{—}035738). Accordingly, utilities of VGAM117 include diagnosis, prevention and treatment of diseases and clinical conditions associated with BPNT1. FLJ21709 (Accession NM_{—}032206) is another VGAM117 host target gene. FLJ21709 BINDING SITE is HOST TARGET binding site found in the 3′ untranslated region of mRNA encoded by FLJ21709, corresponding to a HOST TARGET binding site such as BINDING SITE I, BINDING SITE II or BINDING SITE III. Table 2 illustrates the complementarity of the nucleotide sequences of FLJ21709 BINDING SITE, designated SEQ ID:2235, to the nucleotide sequence of VGAM117 RNA, herein designated VGAM RNA, also designated SEQ ID:452.

Another function of VGAM117 is therefore inhibition of FLJ21709 (Accession NM_{—}032206). Accordingly, utilities of VGAM117 include diagnosis, prevention and treatment of diseases and clinical conditions associated with FLJ21709. MGC13007 (Accession NM_{—}032320) is another VGAM117 host target gene. MGC13007 BINDING SITE is HOST TARGET binding site found in the 5′ untranslated region of mRNA encoded by MGC13007, corresponding to a HOST TARGET binding site such as BINDING SITE I, BINDING SITE II or BINDING SITE III. Table 2 illustrates the complementarity of the nucleotide sequences of MGC13007 BINDING SITE, designated SEQ ID:2246, to the nucleotide sequence of VGAM117 RNA, herein designated VGAM RNA, also designated SEQ ID:452.

Another function of VGAM117 is therefore inhibition of MGC13007 (Accession NM_{—}032320). Accordingly, utilities of VGAM117 include diagnosis, prevention and treatment of diseases and clinical conditions associated with MGC13007. MGC14407 (Accession NM_{—}032908) is another VGAM117 host target gene. MGC14407 BINDING SITE is HOST TARGET binding site found in the 5′ untranslated region of mRNA encoded by MGC14407, corresponding to a HOST TARGET binding site such as BINDING SITE I, BINDING SITE II or BINDING SITE III. Table 2 illustrates the complementarity of the nucleotide sequences of MGC14407 BINDING SITE, designated SEQ ID:2294, to the nucleotide sequence of VGAM117 RNA, herein designated VGAM RNA, also designated SEQ ID:452.

Another function of VGAM117 is therefore inhibition of MGC14407 (Accession NM_{—}032908). Accordingly, utilities of VGAM117 include diagnosis, prevention and treatment of diseases and clinical conditions associated with MGC14407. LOC151201 (Accession XM_{—}098021) is another VGAM117 host target gene. LOC151201 BINDING SITE is HOST TARGET binding site found in the 3′ untranslated region of mRNA encoded by LOC151201, corresponding to a HOST TARGET binding site such as BINDING SITE I, BINDING SITE II or BINDING SITE III. Table 2 illustrates the complementarity of the nucleotide sequences of LOC151201 BINDING SITE, designated SEQ ID:3341, to the nucleotide sequence of VGAM117 RNA, herein designated VGAM RNA, also designated SEQ ID:452.

Another function of VGAM117 is therefore inhibition of LOC151201 (Accession XM_{—}098021). Accordingly, utilities of VGAM117 include diagnosis, prevention and treatment of diseases and clinical conditions associated with LOC151201. LOC197131 (Accession XM_{—}113823) is another VGAM117 host target gene. LOC197131 BINDING SITE is HOST TARGET binding site found in the 3′ untranslated region of mRNA encoded by LOC197131, corresponding to a HOST TARGET binding site such as BINDING SITE I, BINDING SITE II or BINDING SITE III. Table 2 illustrates the complementarity of the nucleotide sequences of LOC197131 BINDING SITE, designated SEQ ID:3419, to the nucleotide sequence of VGAM117 RNA, herein designated VGAM RNA, also designated SEQ ID:452.

Another function of VGAM117 is therefore inhibition of LOC197131 (Accession XM_{—}113823). Accordingly, utilities of VGAM117 include diagnosis, prevention and treatment of diseases and clinical conditions associated with LOC197131. LOC219942 (Accession XM_{—}167790) is another VGAM117 host target gene. LOC219942 BINDING SITE is HOST TARGET binding site found in the 3′ untranslated region of mRNA encoded by LOC219942, corresponding to a HOST TARGET binding site such as BINDING SITE I, BINDING SITE II or BINDING SITE III. Table 2 illustrates the complementarity of the nucleotide sequences of LOC219942 BINDING SITE, designated SEQ ID:3598, to the nucleotide sequence of VGAM117 RNA, herein designated VGAM RNA, also designated SEQ ID:452.

Another function of VGAM117 is therefore inhibition of LOC219942 (Accession XM_{—}167790). Accordingly, utilities of VGAM117 include diagnosis, prevention and treatment of diseases and clinical conditions associated with LOC219942. FIG. 1 further provides a conceptual description of a novel bioinformatically detected viral gene of the present invention, referred to here as Viral Genomic Address Messenger 118 (VGAM118) viral gene, which modulates expression of respective host target genes thereof, the function and utility of which host target genes is known in the art.

VGAM118 is a novel bioinformatically detected regulatory, non protein coding, viral micro RNA (miRNA) gene. The method by which VGAM118 was detected is described hereinabove with reference to FIGS. 1-8.

VGAM118 gene, herein designated VGAM GENE, is a viral gene contained in the genome of Vaccinia Virus. VGAM118 host target gene, herein designated VGAM HOST TARGET GENE, is a human gene contained in the human genome.

VGAM118 gene encodes a VGAM118 precursor RNA, herein designated VGAM PRECURSOR RNA. Similar to other miRNA genes, and unlike most ordinary genes, VGAM118 precursor RNA does not encode a protein. A nucleotide sequence identical or highly similar to the nucleotide sequence of VGAM118 precursor RNA is designated SEQ ID:104, and is provided hereinbelow with reference to the sequence listing part. Nucleotide sequence SEQ ID: 104 is located at position 50980 relative to the genome of Vaccinia Virus.

VGAM118 precursor RNA folds onto itself, forming VGAM118 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM118 folded precursor RNA into VGAM118 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM118 RNA is designated SEQ ID:453, and is provided hereinbelow with reference to the sequence listing part.

VGAM118 host target gene, herein designated VGAM HOST TARGET GENE, encodes a corresponding messenger RNA, VGAM118 host target RNA, herein designated VGAM HOST TARGET RNA. VGAM118 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM118 RNA, herein designated VGAM RNA, binds complementarily to one or more host target binding sites located in untranslated regions of VGAM118 host target RNA, herein designated VGAM HOST TARGET RNA. This complementary binding is due to the fact that the nucleotide sequence of VGAM118 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM118 RNA, herein designated VGAM RNA, may have a different number of host target binding sites in untranslated regions of a VGAM118 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM118 RNA, herein designated VGAM RNA, to host target binding sites on VGAM118 host target RNA, herein designated VGAM HOST TARGET RNA, such as BINDING SITE I, BINDING SITE II and BINDING SITE III, inhibits translation of VGAM118 host target RNA into VGAM118 host target protein, herein designated VGAM HOST TARGET PROTEIN. VGAM host target protein is therefore outlined by a broken line.

It is appreciated that VGAM118 host target gene, herein designated VGAM HOST TARGET GENE, in fact represents a plurality of VGAM118 host target genes. The mRNA of each one of this plurality of VGAM118 host target genes comprises one or more host target binding sites, each having a nucleotide sequence which is at least partly complementary to VGAM118 RNA, herein designated VGAM RNA, and which when bound by VGAM118 RNA causes inhibition of translation of respective one or more VGAM118 host target proteins.

It is further appreciated by one skilled in the art that the mode of translational inhibition illustrated by FIG. 1 with specific reference to translational inhibition exerted by VGAM118 gene, herein designated VGAM GENE, on one or more VGAM118 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM118 is inhibition of expression of host target genes, as part of a novel viral mechanism of attacking a host. Accordingly, utilities of VGAM118 include diagnosis, prevention and treatment of viral infection by Vaccinia Virus. Specific functions, and accordingly utilities, of VGAM118 correlate with, and may be deduced from, the identity of the host target genes which VGAM118 binds and inhibits, and the function of these host target genes, as elaborated hereinbelow.

Nucleotide sequences of the VGAM118 precursor RNA, herein designated VGAM PRECURSOR RNA, and of the ‘diced’ VGAM118 RNA, herein designated VGAM RNA, and a schematic representation of the secondary folding of VGAM118 folded precursor RNA, herein designated VGAM FOLDED PRECURSOR RNA, of VGAM118 are further described hereinbelow with reference to Table 1.

Nucleotide sequences of host target binding sites, such as BINDING SITE-I, BINDING SITE-II and BINDING SITE-III of FIG. 1, found on VGAM118 host target RNA, and schematic representation of the complementarity of each of these host target binding sites to VGAM118 RNA, herein designated VGAM RNA, are described hereinbelow with reference to Table 2.

As mentioned hereinabove with reference to FIG. 1, a function of VGAM118 gene, herein designated VGAM is inhibition of expression of VGAM118 target genes. It is appreciated that specific functions, and accordingly utilities, of VGAM118 correlate with, and may be deduced from, the identity of the target genes which VGAM118 binds and inhibits, and the function of these target genes, as elaborated hereinbelow.

Beta-1,3-glucuronyltransferase 1 (glucuronosyltransferase P) (B3GAT1, Accession NM_{—}018644) is a VGAM118 host target gene. B3GAT1 BINDING SITE1 and B3GAT1 BINDING SITE2 are HOST TARGET binding sites found in untranslated regions of mRNA encoded by B3GAT1, corresponding to HOST TARGET binding sites such as BINDING SITE I, BINDING SITE II or BINDING SITE III. Table 2 illustrates the complementarity of the nucleotide sequences of B3GAT1 BINDING SITE1 and B3GAT1 BINDING SITE2, designated SEQ ID:1855 and SEQ ID:2362 respectively, to the nucleotide sequence of VGAM118 RNA, herein designated VGAM RNA, also designated SEQ ID:453.

A function of VGAM118 is therefore inhibition of Beta-1,3-glucuronyltransferase 1 (glucuronosyltransferase P) (B3GAT1, Accession NM_{—}018644). Accordingly, utilities of VGAM118 include diagnosis, prevention and treatment of diseases and clinical conditions associated with B3GAT1. Zinc Finger Protein 76 (expressed in testis) (ZNF76, Accession NM_{—}003427) is another VGAM118 host target gene. ZNF76 BINDING SITE is HOST TARGET binding site found in the 3′ untranslated region of mRNA encoded by ZNF76, corresponding to a HOST TARGET binding site such as BINDING SITE I, BINDING SITE II or BINDING SITE III. Table 2 illustrates the complementarity of the nucleotide sequences of ZNF76 BINDING SITE, designated SEQ ID:1019, to the nucleotide sequence of VGAM118 RNA, herein designated VGAM RNA, also designated SEQ ID:453.

Another function of VGAM118 is therefore inhibition of Zinc Finger Protein 76 (expressed in testis) (ZNF76, Accession NM_{—}003427). Accordingly, utilities of VGAM118 include diagnosis, prevention and treatment of diseases and clinical conditions associated with ZNF76. DKFZp434MO331 (Accession NM_{—}017600) is another VGAM118 host target gene. DKFZp434MO331 BINDING SITE is HOST TARGET binding site found in the 5′ untranslated region of mRNA encoded by DKFZp434MO331, corresponding to a HOST TARGET binding site such as BINDING SITE I, BINDING SITE II or BINDING SITE III. Table 2 illustrates the complementarity of the nucleotide sequences of DKFZp434MO331 BINDING SITE, designated SEQ ID:1727, to the nucleotide sequence of VGAM118 RNA, herein designated VGAM RNA, also designated SEQ ID:453.

Another function of VGAM118 is therefore inhibition of DKFZp434MO331 (Accession NM_{—}017600). Accordingly, utilities of VGAM118 include diagnosis, prevention and treatment of diseases and clinical conditions associated with DKFZp434MO331. FLJ20666 (Accession NM_{—}017922) is another VGAM118 host target gene. FLJ20666 BINDING SITE is HOST TARGET binding site found in the 5′ untranslated region of mRNA encoded by FLJ20666, corresponding to a HOST TARGET binding site such as BINDING SITE I, BINDING SITE II or BINDING SITE III. Table 2 illustrates the complementarity of the nucleotide sequences of FLJ20666 BINDING SITE, designated SEQ ID:1763, to the nucleotide sequence of VGAM118 RNA, herein designated VGAM RNA, also designated SEQ ID:453.

Another function of VGAM118 is therefore inhibition of FLJ20666 (Accession NM_{—}017922). Accordingly, utilities of VGAM118 include diagnosis, prevention and treatment of diseases and clinical conditions associated with FLJ20666. FYVE and Coiled-coil Domain Containing 1 (FYCO1, Accession NM_{—}024513) is another VGAM118 host target gene. FYCO1 BINDING SITE is HOST TARGET binding site found in the 3′ untranslated region of mRNA encoded by FYCO1, corresponding to a HOST TARGET binding site such as BINDING SITE I, BINDING SITE II or BINDING SITE III. Table 2 illustrates the complementarity of the nucleotide sequences of FYCO1 BINDING SITE, designated SEQ ID:2064, to the nucleotide sequence of VGAM118 RNA, herein designated VGAM RNA, also designated SEQ ID:453.

Another function of VGAM118 is therefore inhibition of FYVE and Coiled-coil Domain Containing 1 (FYCO1, Accession NM_{—}024513). Accordingly, utilities of VGAM118 include diagnosis, prevention and treatment of diseases and clinical conditions associated with FYCO1. Interleukin Enhancer Binding Factor 3, 90 kDa (ILF3, Accession NM_{—}004516) is another VGAM118 host target gene. ILF3 BINDING SITE is HOST TARGET binding site found in the untranslated region of mRNA encoded by ILF3, corresponding to a HOST TARGET binding site such as BINDING SITE I, BINDING SITE II or BINDING SITE III. Table 2 illustrates the complementarity of the nucleotide sequences of ILF3 BINDING SITE, designated SEQ ID:1121, to the nucleotide sequence of VGAM118 RNA, herein designated VGAM RNA, also designated SEQ ID:453.

Another function of VGAM118 is therefore inhibition of Interleukin Enhancer Binding Factor 3, 90 kDa (ILF3, Accession NM_{—}004516). Accordingly, utilities of VGAM118 include diagnosis, prevention and treatment of diseases and clinical conditions associated with ILF3. KIAA0193 (Accession NM_{—}014766) is another VGAM118 host target gene. KIAA0193 BINDING SITE is HOST TARGET binding site found in the 3′ untranslated region of mRNA encoded by KIAA0193, corresponding to a HOST TARGET binding site such as BINDING SITE I, BINDING SITE II or BINDING SITE III. Table 2 illustrates the complementarity of the nucleotide sequences of KIAA0193 BINDING SITE, designated SEQ ID: 1544, to the nucleotide sequence of VGAM118 RNA, herein designated VGAM RNA, also designated SEQ ID:453.

Another function of VGAM118 is therefore inhibition of KIAA0193 (Accession NM_{—}014766). Accordingly, utilities of VGAM118 include diagnosis, prevention and treatment of diseases and clinical conditions associated with KIAA0193. KIAA1163 (Accession XM_{—}086231) is another VGAM118 host target gene. KIAA1163 BINDING SITE is HOST TARGET binding site found in the 3′ untranslated region of mRNA encoded by KIAA1163, corresponding to a HOST TARGET binding site such as BINDING SITE I, BINDING SITE II or BINDING SITE III. Table 2 illustrates the complementarity of the nucleotide sequences of KIAA1163 BINDING SITE, designated SEQ ID:3121, to the nucleotide sequence of VGAM118 RNA, herein designated VGAM RNA, also designated SEQ ID:453.

Another function of VGAM118 is therefore inhibition of KIAA1163 (Accession XM_{—}086231). Accordingly, utilities of VGAM118 include diagnosis, prevention and treatment of diseases and clinical conditions associated with KIAA1163. KIAA1247 (Accession XM_{—}030036) is another VGAM118 host target gene. KIAA1247 BINDING SITE is HOST TARGET binding site found in the 3′ untranslated region of mRNA encoded by KIAA1247, corresponding to a HOST TARGET binding site such as BINDING SITE I, BINDING SITE II or BINDING SITE III. Table 2 illustrates the complementarity of the nucleotide sequences of KIAA1247 BINDING SITE, designated SEQ ID:2609, to the nucleotide sequence of VGAM118 RNA, herein designated VGAM RNA, also designated SEQ ID:453.

Another function of VGAM118 is therefore inhibition of KIAA1247 (Accession XM_{—}030036). Accordingly, utilities of VGAM118 include diagnosis, prevention and treatment of diseases and clinical conditions associated with KIAA1247. KIAA1870 (Accession NM_{—}032888) is another VGAM118 host target gene. KIAA1870 BINDING SITE is HOST TARGET binding site found in the 5′ untranslated region of mRNA encoded by KIAA1870, corresponding to a HOST TARGET binding site such as BINDING SITE I, BINDING SITE II or BINDING SITE III. Table 2 illustrates the complementarity of the nucleotide sequences of KIAA1870 BINDING SITE, designated SEQ ID:2293, to the nucleotide sequence of VGAM118 RNA, herein designated VGAM RNA, also designated SEQ ID:453.

Another function of VGAM118 is therefore inhibition of KIAA1870 (Accession NM_{—}032888). Accordingly, utilities of VGAM118 include diagnosis, prevention and treatment of diseases and clinical conditions associated with KIAA1870. LOC146517 (Accession XM_{—}085491) is another VGAM118 host target gene. LOC146517 BINDING SITE is HOST TARGET binding site found in the 3′ untranslated region of mRNA encoded by LOC146517, corresponding to a HOST TARGET binding site such as BINDING SITE I, BINDING SITE II or BINDING SITE III. Table 2 illustrates the complementarity of the nucleotide sequences of LOC146517 BINDING SITE, designated SEQ ID:3095, to the nucleotide sequence of VGAM118 RNA, herein designated VGAM RNA, also designated SEQ ID:453.

Another function of VGAM118 is therefore inhibition of LOC146517 (Accession XM_{—}085491). Accordingly, utilities of VGAM118 include diagnosis, prevention and treatment of diseases and clinical conditions associated with LOC146517. LOC162083 (Accession XM_{—}091339) is another VGAM118 host target gene. LOC162083 BINDING SITE is HOST TARGET binding site found in the 5′ untranslated region of mRNA encoded by LOC162083, corresponding to a HOST TARGET binding site such as BINDING SITE I, BINDING SITE II or BINDING SITE III. Table 2 illustrates the complementarity of the nucleotide sequences of LOC162083 BINDING SITE, designated SEQ ID:3242, to the nucleotide sequence of VGAM118 RNA, herein designated VGAM RNA, also designated SEQ ID:453.

Another function of VGAM118 is therefore inhibition of LOC162083 (Accession XM_{—}091339). Accordingly, utilities of VGAM118 include diagnosis, prevention and treatment of diseases and clinical conditions associated with LOC162083. LOC220477 (Accession XM_{—}071675) is another VGAM118 host target gene. LOC220477 BINDING SITE is HOST TARGET binding site found in the 5′ untranslated region of mRNA encoded by LOC220477, corresponding to a HOST TARGET binding site such as BINDING SITE I, BINDING SITE II or BINDING SITE III. Table 2 illustrates the complementarity of the nucleotide sequences of LOC220477 BINDING SITE, designated SEQ ID:3041, to the nucleotide sequence of VGAM118 RNA, herein designated VGAM RNA, also designated SEQ ID:453.

Another function of VGAM118 is therefore inhibition of LOC220477 (Accession XM_{—}071675). Accordingly, utilities of VGAM118 include diagnosis, prevention and treatment of diseases and clinical conditions associated with LOC220477. LOC222237 (Accession XM_{—}168592) is another VGAM118 host target gene. LOC222237 BINDING SITE is HOST TARGET binding site found in the 3′ untranslated region of mRNA encoded by LOC222237, corresponding to a HOST TARGET binding site such as BINDING SITE I, BINDING SITE II or BINDING SITE III. Table 2 illustrates the complementarity of the nucleotide sequences of LOC222237 BINDING SITE, designated SEQ ID:3650, to the nucleotide sequence of VGAM118 RNA, herein designated VGAM RNA, also designated SEQ ID:453.

Another function of VGAM118 is therefore inhibition of LOC222237 (Accession XM_{—}168592). Accordingly, utilities of VGAM118 include diagnosis, prevention and treatment of diseases and clinical conditions associated with LOC222237. LOC255096 (Accession XM_{—}174913) is another VGAM118 host target gene. LOC255096 BINDING SITE is HOST TARGET binding site found in the 5′ untranslated region of mRNA encoded by LOC255096, corresponding to a HOST TARGET binding site such as BINDING SITE I, BINDING SITE II or BINDING SITE III. Table 2 illustrates the complementarity of the nucleotide sequences of LOC255096 BINDING SITE, designated SEQ ID:3745, to the nucleotide sequence of VGAM118 RNA, herein designated VGAM RNA, also designated SEQ ID:453.

Another function of VGAM118 is therefore inhibition of LOC255096 (Accession XM_{—}174913). Accordingly, utilities of VGAM118 include diagnosis, prevention and treatment of diseases and clinical conditions associated with LOC255096. LOC92340 (Accession XM_{—}044426) is another VGAM118 host target gene. LOC92340 BINDING SITE is HOST TARGET binding site found in the 3′ untranslated region of mRNA encoded by LOC92340, corresponding to a HOST TARGET binding site such as BINDING SITE I, BINDING SITE II or BINDING SITE III. Table 2 illustrates the complementarity of the nucleotide sequences of LOC92340 BINDING SITE, designated SEQ ID:2837, to the nucleotide sequence of VGAM118 RNA, herein designated VGAM RNA, also designated SEQ ID:453.

Another function of VGAM118 is therefore inhibition of LOC92340 (Accession XM_{—}044426). Accordingly, utilities of VGAM118 include diagnosis, prevention and treatment of diseases and clinical conditions associated with LOC92340. FIG. 1 further provides a conceptual description of a novel bioinformatically detected viral gene of the present invention, referred to here as Viral Genomic Address Messenger 119 (VGAM119) viral gene, which modulates expression of respective host target genes thereof, the function and utility of which host target genes is known in the art.

VGAM119 is a novel bioinformatically detected regulatory, non protein coding, viral micro RNA (miRNA) gene. The method by which VGAM119 was detected is described hereinabove with reference to FIGS. 1-8.

VGAM119 gene, herein designated VGAM GENE, is a viral gene contained in the genome of Vaccinia Virus. VGAM119 host target gene, herein designated VGAM HOST TARGET GENE, is a human gene contained in the human genome.

VGAM119 gene encodes a VGAM119 precursor RNA, herein designated VGAM PRECURSOR RNA. Similar to other miRNA genes, and unlike most ordinary genes, VGAM119 precursor RNA does not encode a protein. A nucleotide sequence identical or highly similar to the nucleotide sequence of VGAM119 precursor RNA is designated SEQ ID:105, and is provided hereinbelow with reference to the sequence listing part. Nucleotide sequence SEQ ID: 105 is located at position 52159 relative to the genome of Vaccinia Virus.

VGAM119 precursor RNA folds onto itself, forming VGAM119 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM119 folded precursor RNA into VGAM119 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM119 RNA is designated SEQ ID:454, and is provided hereinbelow with reference to the sequence listing part.

VGAM119 host target gene, herein designated VGAM HOST TARGET GENE, encodes a corresponding messenger RNA, VGAM119 host target RNA, herein designated VGAM HOST TARGET RNA. VGAM119 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM119 RNA, herein designated VGAM RNA, binds complementarily to one or more host target binding sites located in untranslated regions of VGAM119 host target RNA, herein designated VGAM HOST TARGET RNA. This complementary binding is due to the fact that the nucleotide sequence of VGAM119 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM119 RNA, herein designated VGAM RNA, may have a different number of host target binding sites in untranslated regions of a VGAM119 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM119 RNA, herein designated VGAM RNA, to host target binding sites on VGAM119 host target RNA, herein designated VGAM HOST TARGET RNA, such as BINDING SITE I, BINDING SITE II and BINDING SITE III, inhibits translation of VGAM119 host target RNA into VGAM119 host target protein, herein designated VGAM HOST TARGET PROTEIN. VGAM host target protein is therefore outlined by a broken line.

It is appreciated that VGAM119 host target gene, herein designated VGAM HOST TARGET GENE, in fact represents a plurality of VGAM119 host target genes. The mRNA of each one of this plurality of VGAM119 host target genes comprises one or more host target binding sites, each having a nucleotide sequence which is at least partly complementary to VGAM119 RNA, herein designated VGAM RNA, and which when bound by VGAM119 RNA causes inhibition of translation of respective one or more VGAM119 host target proteins.

It is further appreciated by one skilled in the art that the mode of translational inhibition illustrated by FIG. 1 with specific reference to translational inhibition exerted by VGAM119 gene, herein designated VGAM GENE, on one or more VGAM119 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM119 is inhibition of expression of host target genes, as part of a novel viral mechanism of attacking a host. Accordingly, utilities of VGAM119 include diagnosis, prevention and treatment of viral infection by Vaccinia Virus. Specific functions, and accordingly utilities, of VGAM119 correlate with, and may be deduced from, the identity of the host target genes which VGAM119 binds and inhibits, and the function of these host target genes, as elaborated hereinbelow.

Nucleotide sequences of the VGAM119 precursor RNA, herein designated VGAM PRECURSOR RNA, and of the ‘diced’ VGAM119 RNA, herein designated VGAM RNA, and a schematic representation of the secondary folding of VGAM119 folded precursor RNA, herein designated VGAM FOLDED PRECURSOR RNA, of VGAM119 are further described hereinbelow with reference to Table 1.

Nucleotide sequences of host target binding sites, such as BINDING SITE-I, BINDING SITE-II and BINDING SITE-III of FIG. 1, found on VGAM119 host target RNA, and schematic representation of the complementarity of each of these host target binding sites to VGAM119 RNA, herein designated VGAM RNA, are described hereinbelow with reference to Table 2.

As mentioned hereinabove with reference to FIG. 1, a function of VGAM119 gene, herein designated VGAM is inhibition of expression of VGAM119 target genes. It is appreciated that specific functions, and accordingly utilities, of VGAM119 correlate with, and may be deduced from, the identity of the target genes which VGAM119 binds and inhibits, and the function of these target genes, as elaborated hereinbelow.

Arginase, Liver (ARG1, Accession NM_{—}000045) is a VGAM119 host target gene. ARG1 BINDING SITE is HOST TARGET binding site found in the 3′ untranslated region of mRNA encoded by ARG1, corresponding to a HOST TARGET binding site such as BINDING SITE I, BINDING SITE II or BINDING SITE III. Table 2 illustrates the complementarity of the nucleotide sequences of ARG1 BINDING SITE, designated SEQ ID:700, to the nucleotide sequence of VGAM119 RNA, herein designated VGAM RNA, also designated SEQ ID:454.

A function of VGAM119 is therefore inhibition of Arginase, Liver (ARG1, Accession NM_{—}000045). Accordingly, utilities of VGAM119 include diagnosis, prevention and treatment of diseases and clinical conditions associated with ARG1. BTB and CNC Homology 1, Basic Leucine Zipper Transcription Factor 2 (BACH2, Accession NM_{—}021813) is another VGAM119 host target gene. BACH2 BINDING SITE is HOST TARGET binding site found in the 3′ untranslated region of mRNA encoded by BACH2, corresponding to a HOST TARGET binding site such as BINDING SITE I, BINDING SITE II or BINDING SITE III. Table 2 illustrates the complementarity of the nucleotide sequences of BACH2 BINDING SITE, designated SEQ ID:1964, to the nucleotide sequence of VGAM119 RNA, herein designated VGAM RNA, also designated SEQ ID:454.

Another function of VGAM119 is therefore inhibition of BTB and CNC Homology 1, Basic Leucine Zipper Transcription Factor 2 (BACH2, Accession NM_{—}021813), a gene which acts as repressor or activator, binds to maf recognition elements. Accordingly, utilities of VGAM119 include diagnosis, prevention and treatment of diseases and clinical conditions associated with BACH2. The function of BACH2 has been established by previous studies. By screening a K562 erythroleukemia cell line with mouse Bach2 cDNA as the probe, Sasaki et al. (2000) isolated a cDNA encoding BACH2. The deduced 841-amino acid protein is 89.5% identical to mouse Bach2, with 97% identity shared in the BTB and bZip functional domains and 94% identity shared in the serine-rich region. Northern blot analysis revealed expression of an approximately 11.0-kb BACH2 transcript restricted to thymus, spleen, and leukocytes; low levels were also detected in small intestine and brain. Sasaki et al. (2000) found mRNA and protein expression primarily in B-lymphoid rather than other hematopoietic cell lines. RT-PCR analysis showed that BACH2, like mouse Bach2, is expressed in primary B cells at the progenitor, precursor, immature, and mature B-cell stages. Mouse Bach2 is not expressed in plasma cells (Muto et al., 1998). Gel shift analysis showed that when overexpressed, BACH2 binds to MAF recognition elements (MARE). Overexpression also resulted in a loss of clonogenic activity. Southern blot analysis determined that BACH2 is a single-copy gene. BACH2/CA-1 microsatellite analysis indicated that loss of heterozygosity occurred in 5 of 25 non-Hodgkin lymphoma (OMIM Ref. No. 605027) patients.

Full details of the abovementioned studies are described in the following publications, the disclosure of which are hereby incorporated by reference:
Sasaki, S.; Ito, E.; Toki, T.; Maekawa, T.; Kanezaki, R.; Umenai, T.; Muto, A.; Nagai, H.; Kinoshita, T.; Yamamoto, M.; Inazawa, J.; Taketo, M. M.; Nakahata, T.; Igarashi, K.; Yokoyama, M.: Cloning and expression of human B cell-specific transcription factor BACH2 mapped to chromosome 6q15. Oncogene 19: 3739-3749, 2000.; and
Muto, A.; Hoshino, H.; Madisen, L.; Yanai, N.; Obinata, M.; Karasuyama, H.; Hayashi, N.; Nakauchi, H.; Yamamoto, M.; Groudine, M.; Igarashi, K.: Identification of Bach2 as a B-cell-spe.

Further studies establishing the function and utilities of BACH2 are found in John Hopkins OMIM database record ID 605394, and in sited publications numbered 165 and 1658 listed in the bibliography section hereinbelow, which are also hereby incorporated by reference. Cerebellar Degeneration-related Protein 1, 34 kDa (CDR1, Accession NM_{—}004065) is another VGAM119 host target gene. CDR1 BINDING SITE is HOST TARGET binding site found in the 3′ untranslated region of mRNA encoded by CDR1, corresponding to a HOST TARGET binding site such as BINDING SITE I, BINDING SITE II or BINDING SITE III. Table 2 illustrates the complementarity of the nucleotide sequences of CDR1 BINDING SITE, designated SEQ ID: 1083, to the nucleotide sequence of VGAM119 RNA, herein designated VGAM RNA, also designated SEQ ID:454.

Another function of VGAM119 is therefore inhibition of Cerebellar Degeneration-related Protein 1, 34 kDa (CDR1, Accession NM_{—}004065). Accordingly, utilities of VGAM119 include diagnosis, prevention and treatment of diseases and clinical conditions associated with CDR1. Contactin 3 (plasmacytoma associated) (CNTN3, Accession XM_{—}039627) is another VGAM119 host target gene. CNTN3 BINDING SITE is HOST TARGET binding site found in the 3′ untranslated region of mRNA encoded by CNTN3, corresponding to a HOST TARGET binding site such as BINDING SITE I, BINDING SITE II or BINDING SITE III. Table 2 illustrates the complementarity of the nucleotide sequences of CNTN3 BINDING SITE, designated SEQ ID:2758, to the nucleotide sequence of VGAM119 RNA, herein designated VGAM RNA, also designated SEQ ID:454.

Another function of VGAM119 is therefore inhibition of Contactin 3 (plasmacytoma associated) (CNTN3, Accession XM_{—}039627), a gene which may play a role in the initial growth and guidance of axons. Accordingly, utilities of VGAM119 include diagnosis, prevention and treatment of diseases and clinical conditions associated with CNTN3. The function of CNTN3 has been established by previous studies. Pang, a mouse gene encoding a neuronal adhesion molecule, was isolated as a plasmacytoma-specific transcript using an RT-PCR-based strategy in an attempt to isolate Myc-like genes in murine plasmacytomas (Connelly et al., 1994). Pang is a member of the immunoglobulin/fibronectin super family of adhesion molecules; its closest relatives, TAG1 (OMIM Ref. No. 190197) and contactin 1 (OMIM Ref. No. 600016), promote axon growth and migration. The normal site of Pang expression is the brain, where it is detected as 4.0- and 6.1-kb RNAs on Northern blots; Pang is not detected in other normal tissues. Abnormally sized Pang transcripts were uniquely found in murine plasmacytomas, where it is ectopically activated by intracisternal A-type particle long terminal repeats. Mock et al. (1996) mapped the Pang gene to mouse chromosome 6 by somatic cell hybrid analysis and further positioned it on the chromosome between Wnt7a and Pcp1. Southern blot analysis of human-rodent somatic cell hybrids together with predictions from the mouse map location indicated that human PANG is located at 3p26

Full details of the abovementioned studies are described in the following publications, the disclosure of which are hereby incorporated by reference:
Connelly, M. A.; Grady, R. C.; Mushinski, J. F.; Marcu, K. B.: PANG, a gene encoding a neuronal glycoprotein, is ectopically activated by intracisternal A-type particle long terminal repeats in murine plasmacytomas. Proc. Nat. Acad. Sci. 91: 1337-1341, 1994.; and
Mock, B. A.; Connelly, M. A.; McBride, O. W.; Kozak, C. A.; Marcu, K. B.: Plasmacytoma-associated neuronal glycoprotein, Pang, maps to mouse chromosome 6 and human chromosome 3. Genomic.

Further studies establishing the function and utilities of CNTN3 are found in John Hopkins OMIM database record ID 601325, and in sited publications numbered 2122-2123 listed in the bibliography section hereinbelow, which are also hereby incorporated by reference. Estrogen-related Receptor Gamma (ESRRG, Accession XM_{—}039053) is another VGAM119 host target gene. ESRRG BINDING SITE is HOST TARGET binding site found in the 3′ untranslated region of mRNA encoded by ESRRG, corresponding to a HOST TARGET binding site such as BINDING SITE I, BINDING SITE II or BINDING SITE III. Table 2 illustrates the complementarity of the nucleotide sequences of ESRRG BINDING SITE, designated SEQ ID:2748, to the nucleotide sequence of VGAM119 RNA, herein designated VGAM RNA, also designated SEQ ID:454.

Another function of VGAM119 is therefore inhibition of Estrogen-related Receptor Gamma (ESRRG, Accession XM_{—}039053), a gene which Estrogen-related receptor gamma. Accordingly, utilities of VGAM119 include diagnosis, prevention and treatment of diseases and clinical conditions associated with ESRRG. The function of ESRRG has been established by previous studies. Members of the nuclear receptor super family are important regulators of development, cell proliferation, and physiology. During an analysis of the critical region of type IIa Usher syndrome (USH2A; 276901) at 1q41, Eudy et al. (1998) constructed a cDNA contig of ESRRG. Northern blot analysis detected a 5.5-kb ESRRG transcript in a variety of human adult and fetal tissues, with the highest level in fetal brain. The predicted 436-amino acid ESRRG protein, which is a member of the steroid/thyroid/retinoid receptor super family, is 76% identical to the orphan receptor ESRRB (OMIM Ref. No. 602167) and 63% identical to ESRRA (OMIM Ref. No. 601998). Heard et al. (2000) reported that the ESRRG mRNA is highly alternatively spliced at the 5-prime end, giving rise to a number of tissue-specific RNA species, some of which encode protein isoforms differing in the N-terminal region. Like ESRRA and ESRRB, ESRRG binds as a monomer to an ERR-alpha response element (ERRE). Hong et al. (1999) identified mouse Esrrg, which they called Err3, by yeast 2-hybrid screening using the transcriptional coactivator GRIP1 (OMIM Ref. No. 604597) as bait. The putative full-length mouse Err3 contains 458 amino acids and is closely related to Err1 and Err2. All ERR family members share an almost identical DNA-binding domain, which shares 68% amino acid identity with that of estrogen receptor. Expression of Err3 in adult mouse was restricted; highest expression was observed in heart, kidney, and brain. In mouse embryo, no expression was observed at day 7, and highest expression occurred around days 11 to 15. Although Err3 is more closely related to Err2 than to Err1, the expression pattern for Err3 was similar to that of Err1 and distinct from that for Err2, suggesting a unique role for Err3 in development. Eudy et al. (1998) mapped the ESRRG gene to the USH2A critical region on chromosome 1q41.

Full details of the abovementioned studies are described in the following publications, the disclosure of which are hereby incorporated by reference:
Heard, D. J.; Norby, P. L.; Holloway, J.; Vissing, H.: Human ERR-gamma, a third member of the estrogen receptor-related receptor (ERR) sub family of orphan nuclear receptors: tissue-specific isoforms are expressed during development in the adult. Molec. Endocr. 14: 382-392, 2000.; and
Eudy, J. D.; Yao, S.; Weston, M. D.; Ma-Edmonds, M.; Talmadge, C. B.; Cheng, J. J.; Kimberling, W. J.; Sumegi, J.: Isolation of a gene encoding a novel member of the nuclear receptor s.

Further studies establishing the function and utilities of ESRRG are found in John Hopkins OMIM database record ID 602969, and in sited publications numbered 1916-191 and 1925-1926 listed in the bibliography section hereinbelow, which are also hereby incorporated by reference. Follistatin-like 1 (FSTL1, Accession NM_{—}007085) is another VGAM119 host target gene. FSTL1 BINDING SITE is HOST TARGET binding site found in the 3′ untranslated region of mRNA encoded by FSTL1, corresponding to a HOST TARGET binding site such as BINDING SITE I, BINDING SITE II or BINDING SITE III. Table 2 illustrates the complementarity of the nucleotide sequences of FSTL1 BINDING SITE, designated SEQ ID:1357, to the nucleotide sequence of VGAM119 RNA, herein designated VGAM RNA, also designated SEQ ID:454.

Another function of VGAM119 is therefore inhibition of Follistatin-like 1 (FSTL1, Accession NM_{—}007085), a gene which may modulate the action of some growth factors on cell proliferation and differentiation. Accordingly, utilities of VGAM119 include diagnosis, prevention and treatment of diseases and clinical conditions associated with FSTL1. The function of FSTL1 has been established by previous studies. Using degenerate primers designed against a peptide purified from a rat glioma cell line, Zwijsen et al. (1994) isolated a full-length follistatin-like cDNA (FSTL1), which they called FRP, from a human glioma cDNA library. FSTL1 encodes a deduced 308-amino acid protein with an N-terminal signal peptide of 20 amino acids. FSTL1 contains an FS module, a follistatin-like sequence containing 10 conserved cysteine residues. The number and distribution of the cysteine residues supports the existence of several intramolecular disulfide bridges. Zwijsen et al. (1994) did not detect any membrane-spanning or membrane-associated sequences in the FSTL1 sequence, but they predicted 3 putative N-glycosylation sites and several phosphorylation sites. Under denaturing conditions, Zwijsen et al. (1994) detected several isoforms of FSTL1 with molecular masses of 40 to 48 kD which differs from the 50- to 55-kD products detected by Tanaka et al. (1998). Tanaka et al. (1998) hypothesized that the difference results from the molecular conditions affected by posttranslational modification. FSTL1 shares greater than 92% amino acid identity with the mouse homolog, known as FstI or TSC-36, identified as a transforming growth factor-beta-inducible protein by Shibanuma et al. (1993). Zwijsen et al. (1994) also noted sequence similarity to follistatin (OMIM Ref. No. 136470) and agrin (OMIM Ref. No. 103320), and could not detect any effect of FSTL1 on the cell growth inhibition of TGF-beta (OMIM Ref. No. 190180). Using Northern blot analysis, Tanaka et al. (1998) detected a broadly expressed 4.4-kb FSTL1 transcript most strongly in the heart, placenta, prostate, ovary, and small intestine. Expression was not detected in peripheral blood leukocytes. Tanaka et al. (1998) constructed synovium expression cDNA libraries made from rheumatoid arthritis (RA; 180300) patient-derived synovial cell mRNA. By screening the libraries by IgG purified from synovial fluids from RA patients, they identified FSTL1. Using immunoblotting analysis, they detected anti-FSTL1 antibodies as more frequent in the synovial fluids and serum of RA patients than in patients with other systemic rheumatic diseases or in healthy individuals. patients. Immunoprecipitation analysis showed no difference between these groups in the amount of synovial FSTL1 protein, suggesting an elevated turnover in RA.

Full details of the abovementioned studies are described in the following publications, the disclosure of which are hereby incorporated by reference:
Tanaka, M.; Ozaki, S.; Osakada, F.; Mori, K.; Okubo, M.; Nakao, K.: Cloning of follistatin-related protein as a novel autoantigen in systemic rheumatic diseases. Int. Immun. 10: 1305-1314, 1998.; and
Zwijsen, A.; Blockx, H.; van Arnhem, W.; Willems, J.; Fransen, L.; Devos, K.; Raymackers, J.; van de Voorde, A.; Slegers, H.: Characterization of a rat C6 glioma-secreted follistatin-r.

Further studies establishing the function and utilities of FSTL1 are found in John Hopkins OMIM database record ID 605547, and in sited publications numbered 1489-1491 listed in the bibliography section hereinbelow, which are also hereby incorporated by reference. Gamma-aminobutyric Acid (GABA) A Receptor, Beta 2 (GABRB2, Accession NM_{—}021911) is another VGAM119 host target gene. GABRB2 BINDING SITE1 and GABRB2 BINDING SITE2 are HOST TARGET binding sites found in untranslated regions of mRNA encoded by GABRB2, corresponding to HOST TARGET binding sites such as BINDING SITE I, BINDING SITE II or BINDING SITE III. Table 2 illustrates the complementarity of the nucleotide sequences of GABRB2 BINDING SITE1 and GABRB2 BINDING SITE2, designated SEQ ID:1967 and SEQ ID:776 respectively, to the nucleotide sequence of VGAM119 RNA, herein designated VGAM RNA, also designated SEQ ID:454.

Another function of VGAM119 is therefore inhibition of Gamma-aminobutyric Acid (GABA) A Receptor, Beta 2 (GABRB2, Accession NM_{—}021911). Accordingly, utilities of VGAM119 include diagnosis, prevention and treatment of diseases and clinical conditions associated with GABRB2. Nuclear Receptor Interacting Protein 1 (NRIP1, Accession XM_{—}009699) is another VGAM119 host target gene. NRIP1 BINDING SITE is HOST TARGET binding site found in the 3′ untranslated region of mRNA encoded by NRIP1, corresponding to a HOST TARGET binding site such as BINDING SITE I, BINDING SITE II or BINDING SITE III. Table 2 illustrates the complementarity of the nucleotide sequences of NRIP1 BINDING SITE, designated SEQ ID:2548, to the nucleotide sequence of VGAM119 RNA, herein designated VGAM RNA, also designated SEQ ID:454.

Another function of VGAM119 is therefore inhibition of Nuclear Receptor Interacting Protein 1 (NRIP1, Accession XM_{—}009699), a gene which modulates transcriptional activation by the estrogen receptor. Accordingly, utilities of VGAM119 include diagnosis, prevention and treatment of diseases and clinical conditions associated with NRIP1. The function of NRIP1 has been established by previous studies. Cavailles et al. (1995) identified the receptor-interacting protein 140 (OMIM Ref. No. RIP140) by virtue of its direct association with a transcriptional activation domain of the estrogen receptor (ESR; 133430) in the presence of estrogen; by fluorescence in situ hybridization with a cDNA clone, they mapped the gene to 21q11. Katsanis et al. (1998) used hybrids, YACs, and PACs to place the RIP140 gene on the physical map of chromosome 21; 21q11 is a gene-poor region.

Full details of the abovementioned studies are described in the following publications, the disclosure of which are hereby incorporated by reference:
Cavailles, V.; Dauvois, S.; Horset, L. F.; Lopez, G.; Hoare, S.; Kushner, P. J.; Parker, M. G.: Nuclear factor RIP140 modulates transcriptional activation by the estrogen receptor. EMBO J. 14: 3741-3751, 1995.; and
Katsanis, N.; Ives, J. H.; Groet, J.; Nizetic, D.; Fisher, E. M. C.: Localisation of receptor interacting protein 140 (RIP140) within 100 kb of D21S13 on 21q11, a gene-poor region of t.

Further studies establishing the function and utilities of NRIP1 are found in John Hopkins OMIM database record ID 602490, and in sited publications numbered 233-234 listed in the bibliography section hereinbelow, which are also hereby incorporated by reference. Oligophrenin 1 (OPHN1, Accession NM_{—}002547) is another VGAM119 host target gene. OPHN1 BINDING SITE is HOST TARGET binding site found in the 3′ untranslated region of mRNA encoded by OPHN1, corresponding to a HOST TARGET binding site such as BINDING SITE I, BINDING SITE II or BINDING SITE III. Table 2 illustrates the complementarity of the nucleotide sequences of OPHN1 BINDING SITE, designated SEQ ID:936, to the nucleotide sequence of VGAM119 RNA, herein designated VGAM RNA, also designated SEQ ID:454.

Another function of VGAM119 is therefore inhibition of Oligophrenin 1 (OPHN1, Accession NM_{—}002547). Accordingly, utilities of VGAM119 include diagnosis, prevention and treatment of diseases and clinical conditions associated with OPHN1. Protein Tyrosine Phosphatase, Receptor Type, 0 (PTPRO, Accession NM_{—}002848) is another VGAM119 host target gene. PTPRO BINDING SITE1 through PTPRO BINDING SITE5 are HOST TARGET binding sites found in untranslated regions of mRNA encoded by PTPRO, corresponding to HOST TARGET binding sites such as BINDING SITE I, BINDING SITE II or BINDING SITE III. Table 2 illustrates the complementarity of the nucleotide sequences of PTPRO BINDING SITE1 through PTPRO BINDING SITE5, designated SEQ ID:964, SEQ ID:2155, SEQ ID:2156, SEQ ID:2157 and SEQ ID:2158 respectively, to the nucleotide sequence of VGAM119 RNA, herein designated VGAM RNA, also designated SEQ ID:454.

Another function of VGAM119 is therefore inhibition of Protein Tyrosine Phosphatase, Receptor Type, 0 (PTPRO, Accession NM_{—}002848), a gene which may function as a cell contact receptor that mediates and controls cell-cell signals. Accordingly, utilities of VGAM119 include diagnosis, prevention and treatment of diseases and clinical conditions associated with PTPRO. The function of PTPRO has been established by previous studies. To identify protein-tyrosine phosphatases (PTPases) involved in the oncogenic process leading to the development of pancreatic carcinoma, Wang et al. (1996) performed PCR on pooled poly(A)+ RNA from 9 human pancreatic carcinoma cell lines using PTPase consensus oligonucleotide primers. One of the novel PCR products recovered was termed PCP2 (pancreatic carcinoma phosphatase-2) and was used to screen a human pancreatic adenocarcinoma cDNA library. The full sequence of PCP2 predicts a 1,430 amino acid protein consisting of a putative extracellular domain of 740 amino acids, a single transmembrane domain, and an intracellular domain of 666 amino acids. The intracellular region contains 2 tandemly repeated PTP catalytic domains with a high degree of identity to the catalytic domains of mouse PTP-kappa and PTP-mu. In addition to a signal peptide and 13 potential N-linked glycosylation sites, the extracellular domain contains a MAM (meprin/A5/PTP-mu) domain followed by 1 Ig-like repeat and 4 putative fibronectin type III repeats. The MAM domain, found in Xenopus A5 glycoprotein, meprin A, and meprin B, as well as in PTP-kappa and PTP-mu, may be involved in attachment to the cytoskeleton. PCP2, PTP-kappa, and PTP-mu appear to form a sub family of MAM-containing receptor-like PTPs (RPTPs). PCP2 also contains the tripeptide HAV, implicated in cell-cell contact in the cadherins. By Northern blot analysis, Wang et al. (1996) demonstrated that the 5.5-kb PCP2 transcript is widely distributed at varying levels, with very high expression in brain, skeletal muscle, and pancreas, but no expression in placenta or spleen. Wang et al. (1996) demonstrated tyrosine phosphatase activity using an in vitro pNPP assay. Subcellular localization using laser scanning immunofluorescence microscopy showed localization of PCP2 at intercellular adhesions and colocalization with beta-catenin and E-cadherin. Wang et al. (1996) hypothesized that PCP2 and other members of this sub family of RPTPases may function as cell contact receptors that mediate and control cell-cell signals. Wang et al. (1997) used degenerate PCR to clone PTP-J, a member of the type II receptor PTPase family. The PTP-J cDNA encodes a 1,436-amino acid polypeptide that includes a single transmembrane domain and a cytoplasmic domain containing 2 tandemly repeated PTP catalytic domains. The presence of 2 PTP domains indicates that this gene is a member of the type II receptor PTPases. Northern blot analysis detected expression in skeletal muscle, heart, prostate, pancreas, and placenta. Wang et al. (1997) found that in lymphocytes or lymphoma cells, expression of PTP-J is down-regulated following stimulation by either phorbol myristate acetate (PMA) or calcium ionophore, suggesting that PMA or calcium signaling pathways may be involved in regulating the expression of PTP-J.

Full details of the abovementioned studies are described in the following publications, the disclosure of which are hereby incorporated by reference:
Wang, B.; Kishihara, K.; Zhang, D.; Hara, H.; Nomoto, K.: Molecular cloning and characterization of a novel human receptor protein tyrosine phosphatase gene, hPTP-J: down-regulation of gene expression by PMA and calcium ionophore in Jurkat T lymphoma cells. Biochem. Biophys. Res. Commun. 231: 77-81, 1997.; and
Wang, H; Lian, Z; Lerch, M. M.; Chen, Z; Xie, W; Ullrich, A. Characterization of PCP-2, a novel receptor protein tyrosine phosphatase of the MAM domain family. Oncogene 12: 2555-2562.

Further studies establishing the function and utilities of PTPRO are found in John Hopkins OMIM database record ID 602454, and in sited publications numbered 1473-1474 listed in the bibliography section hereinbelow, which are also hereby incorporated by reference. RAR-related Orphan Receptor B (RORB, Accession NM_{—}006914) is another VGAM119 host target gene. RORB BINDING SITE is HOST TARGET binding site found in the 3′ untranslated region of mRNA encoded by RORB, corresponding to a HOST TARGET binding site such as BINDING SITE I, BINDING SITE II or BINDING SITE III. Table 2 illustrates the complementarity of the nucleotide sequences of RORB BINDING SITE, designated SEQ ID:1338, to the nucleotide sequence of VGAM119 RNA, herein designated VGAM RNA, also designated SEQ ID:454.

Another function of VGAM119 is therefore inhibition of RAR-related Orphan Receptor B (RORB, Accession NM_{—}006914), a gene which is an orphan nuclear receptor. Accordingly, utilities of VGAM119 include diagnosis, prevention and treatment of diseases and clinical conditions associated with RORB. The function of RORB has been established by previous studies. ROR-beta is a transcription factor and belongs to the nuclear receptor family (Carlberg et al., 1994). Members of this super family share a common modular structure composed of a transactivation domain, a DNA-binding domain, and a ligand-binding domain (Evans, 1988). Typically, their transcriptional transactivation function is regulated by small lipophilic molecules, such as steroid hormones, vitamin D, retinoic acids, and thyroid hormone. These molecules are synthesized in the organism and pass readily through the plasma membrane to reach the corresponding receptors inside the cell. In addition to the classic hormone receptors, a growing number of nuclear receptors for which no ligands are known have been identified by homology cloning. These nuclear receptors are referred to as ‘orphan’ nuclear receptors. ROR-beta is such an orphan nuclear receptor, forming a sub family with the closely related nuclear receptors ROR-alpha (RORA; 600825) and ROR-gamma (RORC; 602943 Animal model experiments lend further support to the function of RORB. ROR-beta is expressed in areas of the central nervous system that are involved in the processing of sensory information, including spinal cord, thalamus, and sensory cerebellar cortices. Additionally, ROR-beta localizes to the 3 principal anatomic components of the mammalian timing system: the suprachiasmatic nuclei, the retina, and the pineal gland. Andre et al. (1998) showed that RORB mRNA levels oscillate in retina and pineal gland with a circadian rhythm that persists in constant darkness. They generated RORB-deficient mice by gene targeting in embryonic stem cells and analyzed their phenotypic behavior. Rorb −/− mice display a duck-like gait, transient male incapability to reproduce sexually, and a severely disorganized retina that suffers from postnatal degeneration. Consequently, adult Rorb −/− mice are blind, yet their circadian activity rhythm is still entrained by light-dark cycles. Under conditions of constant darkness, Rorb −/− mice display an extended period of free-running rhythmicity. The overall behavioral phenotype of Rorb −/− mice, together with the chromosomal localization of the gene on mouse chromosome 4, suggested a close relationship to the spontaneous mouse mutation ‘vacillans’ described by Sirlin (1956) and now thought to be extinct

It is appreciated that the abovementioned animal model for RORB is acknowledged by those skilled in the art as a scientifically valid animal model, as can be further appreciated from the publications sited hereinbelow.

Full details of the abovementioned studies are described in the following publications, the disclosure of which are hereby incorporated by reference:
Evans, R. M.: The steroid and thyroid hormone receptor super family. Science 240: 889-895, 1988.; and
Andre, E.; Conquet, F.; Steinmayr, M.; Stratton, S. C.; Porciatti, V.; Becker-Andre, M.: Disruption of retinoid-related orphan receptor beta changes circadian behavior, causes retinal deg.

Further studies establishing the function and utilities of RORB are found in John Hopkins OMIM database record ID 601972, and in sited publications numbered 1921, 1922-192 and 1778 listed in the bibliography section hereinbelow, which are also hereby incorporated by reference. Sorbitol Dehydrogenase (SORD, Accession NM_{—}003104) is another VGAM119 host target gene. SORD BINDING SITE is HOST TARGET binding site found in the 3′ untranslated region of mRNA encoded by SORD, corresponding to a HOST TARGET binding site such as BINDING SITE I, BINDING SITE II or BINDING SITE III. Table 2 illustrates the complementarity of the nucleotide sequences of SORD BINDING SITE, designated SEQ ID:990, to the nucleotide sequence of VGAM119 RNA, herein designated VGAM RNA, also designated SEQ ID:454.

Another function of VGAM119 is therefore inhibition of Sorbitol Dehydrogenase (SORD, Accession NM_{—}003104). Accordingly, utilities of VGAM119 include diagnosis, prevention and treatment of diseases and clinical conditions associated with SORD. Sulfotransferase Family, Cytosolic, 1C, Member 1 (SULT1C1, Accession NM_{—}001056) is another VGAM119 host target gene. SULT1C1 BINDING SITE is HOST TARGET binding site found in the 3′ untranslated region of mRNA encoded by SULT1C1, corresponding to a HOST TARGET binding site such as BINDING SITE I, BINDING SITE II or BINDING SITE III. Table 2 illustrates the complementarity of the nucleotide sequences of SULT1C1 BINDING SITE, designated SEQ ID:797, to the nucleotide sequence of VGAM119 RNA, herein designated VGAM RNA, also designated SEQ ID:454.

Another function of VGAM119 is therefore inhibition of Sulfotransferase Family, Cytosolic, 1C, Member 1 (SULT1C1, Accession NM_{—}001056). Accordingly, utilities of VGAM119 include diagnosis, prevention and treatment of diseases and clinical conditions associated with SULT1C1. Tryptophan Rich Basic Protein (WRB, Accession NM_{—}004627) is another VGAM119 host target gene. WRB BINDING SITE is HOST TARGET binding site found in the 3′ untranslated region of mRNA encoded by WRB, corresponding to a HOST TARGET binding site such as BINDING SITE I, BINDING SITE II or BINDING SITE III. Table 2 illustrates the complementarity of the nucleotide sequences of WRB BINDING SITE, designated SEQ ID:1137, to the nucleotide sequence of VGAM119 RNA, herein designated VGAM RNA, also designated SEQ ID:454.

Another function of VGAM119 is therefore inhibition of Tryptophan Rich Basic Protein (WRB, Accession NM_{—}004627). Accordingly, utilities of VGAM119 include diagnosis, prevention and treatment of diseases and clinical conditions associated with WRB. Ankyrin Repeat Domain 5 (ANKRD5, Accession NM_{—}022096) is another VGAM119 host target gene. ANKRD5 BINDING SITE is HOST TARGET binding site found in the 5′ untranslated region of mRNA encoded by ANKRD5, corresponding to a HOST TARGET binding site such as BINDING SITE I, BINDING SITE II or BINDING SITE III. Table 2 illustrates the complementarity of the nucleotide sequences of ANKRD5 BINDING SITE, designated SEQ ID:1978, to the nucleotide sequence of VGAM119 RNA, herein designated VGAM RNA, also designated SEQ ID:454.

Another function of VGAM119 is therefore inhibition of Ankyrin Repeat Domain 5 (ANKRD5, Accession NM_{—}022096). Accordingly, utilities of VGAM119 include diagnosis, prevention and treatment of diseases and clinical conditions associated with ANKRD5. Chromosome 7 Open Reading Frame 10 (C7orf10, Accession NM_{—}024728) is another VGAM119 host target gene. C7orf10 BINDING SITE is HOST TARGET binding site found in the 3′ untranslated region of mRNA encoded by C7orf10, corresponding to a HOST TARGET binding site such as BINDING SITE I, BINDING SITE II or BINDING SITE III. Table 2 illustrates the complementarity of the nucleotide sequences of C7orf10 BINDING SITE, designated SEQ ID:2084, to the nucleotide sequence of VGAM119 RNA, herein designated VGAM RNA, also designated SEQ ID:454.

Another function of VGAM119 is therefore inhibition of Chromosome 7 Open Reading Frame 10 (C7orf10, Accession NM_{—}024728). Accordingly, utilities of VGAM119 include diagnosis, prevention and treatment of diseases and clinical conditions associated with C7orf10. DKFZP727M111 (Accession NM_{—}015540) is another VGAM119 host target gene. DKFZP727M111 BINDING SITE is HOST TARGET binding site found in the 5′ untranslated region of mRNA encoded by DKFZP727M111, corresponding to a HOST TARGET binding site such as BINDING SITE I, BINDING SITE II or BINDING SITE III. Table 2 illustrates the complementarity of the nucleotide sequences of DKFZP727M111 BINDING SITE, designated SEQ ID:1633, to the nucleotide sequence of VGAM119 RNA, herein designated VGAM RNA, also designated SEQ ID:454.

Another function of VGAM119 is therefore inhibition of DKFZP727M111 (Accession NM_{—}015540). Accordingly, utilities of VGAM119 include diagnosis, prevention and treatment of diseases and clinical conditions associated with DKFZP727M111. FLJ13262 (Accession NM_{—}024914) is another VGAM119 host target gene. FLJ13262 BINDING SITE is HOST TARGET binding site found in the 3′ untranslated region of mRNA encoded by FLJ13262, corresponding to a HOST TARGET binding site such as BINDING SITE I, BINDING SITE II or BINDING SITE III. Table 2 illustrates the complementarity of the nucleotide sequences of FLJ13262 BINDING SITE, designated SEQ ID:2113, to the nucleotide sequence of VGAM119 RNA, herein designated VGAM RNA, also designated SEQ ID:454.

Another function of VGAM119 is therefore inhibition of FLJ13262 (Accession NM_{—}024914). Accordingly, utilities of VGAM119 include diagnosis, prevention and treatment of diseases and clinical conditions associated with FLJ13262. FLJ20425 (Accession NM_{—}017816) is another VGAM119 host target gene. FLJ20425 BINDING SITE is HOST TARGET binding site found in the 5′ untranslated region of mRNA encoded by FLJ20425, corresponding to a HOST TARGET binding site such as BINDING SITE I, BINDING SITE II or BINDING SITE III. Table 2 illustrates the complementarity of the nucleotide sequences of FLJ20425 BINDING SITE, designated SEQ ID:1755, to the nucleotide sequence of VGAM119 RNA, herein designated VGAM RNA, also designated SEQ ID:454.

Another function of VGAM119 is therefore inhibition of FLJ20425 (Accession NM_{—}017816). Accordingly, utilities of VGAM119 include diagnosis, prevention and treatment of diseases and clinical conditions associated with FLJ20425. KIAA0953 (Accession XM_{—}039733) is another VGAM119 host target gene. KIAA0953 BINDING SITE is HOST TARGET binding site found in the 3′ untranslated region of mRNA encoded by KIAA0953, corresponding to a HOST TARGET binding site such as BINDING SITE I, BINDING SITE II or BINDING SITE III. Table 2 illustrates the complementarity of the nucleotide sequences of KIAA0953 BINDING SITE, designated SEQ ID:2763, to the nucleotide sequence of VGAM119 RNA, herein designated VGAM RNA, also designated SEQ ID:454.

Another function of VGAM119 is therefore inhibition of KIAA0953 (Accession XM_{—}039733). Accordingly, utilities of VGAM119 include diagnosis, prevention and treatment of diseases and clinical conditions associated with KIAA0953. KIAA1450 (Accession XM_{—}038035) is another VGAM119 host target gene. KIAA1450 BINDING SITE is HOST TARGET binding site found in the 3′ untranslated region of mRNA encoded by KIAA1450, corresponding to a HOST TARGET binding site such as BINDING SITE I, BINDING SITE II or BINDING SITE III. Table 2 illustrates the complementarity of the nucleotide sequences of KIAA1450 BINDING SITE, designated SEQ ID:2732, to the nucleotide sequence of VGAM119 RNA, herein designated VGAM RNA, also designated SEQ ID:454.

Another function of VGAM119 is therefore inhibition of KIAA1450 (Accession XM_{—}038035). Accordingly, utilities of VGAM119 include diagnosis, prevention and treatment of diseases and clinical conditions associated with KIAA1450. Neurogenic Differentiation 6 (NEUROD6, Accession NM_{—}022728) is another VGAM119 host target gene. NEUROD6 BINDING SITE is HOST TARGET binding site found in the 5′ untranslated region of mRNA encoded by NEUROD6, corresponding to a HOST TARGET binding site such as BINDING SITE I, BINDING SITE II or BINDING SITE III. Table 2 illustrates the complementarity of the nucleotide sequences of NEUROD6 BINDING SITE, designated SEQ ID:2000, to the nucleotide sequence of VGAM119 RNA, herein designated VGAM RNA, also designated SEQ ID:454.

Another function of VGAM119 is therefore inhibition of Neurogenic Differentiation 6 (NEUROD6, Accession NM_{—}022728). Accordingly, utilities of VGAM119 include diagnosis, prevention and treatment of diseases and clinical conditions associated with NEUROD6. PRO0255 (Accession NM_{—}014124) is another VGAM119 host target gene. PRO0255 BINDING SITE is HOST TARGET binding site found in the 3′ untranslated region of mRNA encoded by PRO0255, corresponding to a HOST TARGET binding site such as BINDING SITE I, BINDING SITE II or BINDING SITE III. Table 2 illustrates the complementarity of the nucleotide sequences of PRO0255 BINDING SITE, designated SEQ ID: 1475, to the nucleotide sequence of VGAM119 RNA, herein designated VGAM RNA, also designated SEQ ID:454.

Another function of VGAM119 is therefore inhibition of PRO0255 (Accession NM_{—}014124). Accordingly, utilities of VGAM119 include diagnosis, prevention and treatment of diseases and clinical conditions associated with PRO0255. UBE3B (Accession XM_{—}084941) is another VGAM119 host target gene. UBE3B BINDING SITE is HOST TARGET binding site found in the 5′ untranslated region of mRNA encoded by UBE3B, corresponding to a HOST TARGET binding site such as BINDING SITE I, BINDING SITE II or BINDING SITE III. Table 2 illustrates the complementarity of the nucleotide sequences of UBE3B BINDING SITE, designated SEQ ID:3069, to the nucleotide sequence of VGAM119 RNA, herein designated VGAM RNA, also designated SEQ ID:454.

Another function of VGAM119 is therefore inhibition of UBE3B (Accession XM_{—}084941). Accordingly, utilities of VGAM119 include diagnosis, prevention and treatment of diseases and clinical conditions associated with UBE3B. Zinc Finger, Imprinted 2 (ZIM2, Accession NM_{—}015363) is another VGAM119 host target gene. ZIM2 BINDING SITE is HOST TARGET binding site found in the 3′ untranslated region of mRNA encoded by ZIM2, corresponding to a HOST TARGET binding site such as BINDING SITE I, BINDING SITE II or BINDING SITE III. Table 2 illustrates the complementarity of the nucleotide sequences of ZIM2 BINDING SITE, designated SEQ ID: 1623, to the nucleotide sequence of VGAM119 RNA, herein designated VGAM RNA, also designated SEQ ID:454.

Another function of VGAM119 is therefore inhibition of Zinc Finger, Imprinted 2 (ZIM2, Accession NM_{—}015363). Accordingly, utilities of VGAM119 include diagnosis, prevention and treatment of diseases and clinical conditions associated with ZIM2. LOC112868 (Accession XM_{—}053402) is another VGAM119 host target gene. LOC112868 BINDING SITE is HOST TARGET binding site found in the 3′ untranslated region of mRNA encoded by LOC112868, corresponding to a HOST TARGET binding site such as BINDING SITE I, BINDING SITE II or BINDING SITE III. Table 2 illustrates the complementarity of the nucleotide sequences of LOC112868 BINDING SITE, designated SEQ ID:2962, to the nucleotide sequence of VGAM119 RNA, herein designated VGAM RNA, also designated SEQ ID:454.

Another function of VGAM119 is therefore inhibition of LOC112868 (Accession XM_{—}053402). Accordingly, utilities of VGAM119 include diagnosis, prevention and treatment of diseases and clinical conditions associated with LOC112868. LOC116166 (Accession XM_{—}007651) is another VGAM119 host target gene. LOC116166 BINDING SITE is HOST TARGET binding site found in the 3′ untranslated region of mRNA encoded by LOC116166, corresponding to a HOST TARGET binding site such as BINDING SITE I, BINDING SITE II or BINDING SITE III. Table 2 illustrates the complementarity of the nucleotide sequences of LOC116166 BINDING SITE, designated SEQ ID:2545, to the nucleotide sequence of VGAM119 RNA, herein designated VGAM RNA, also designated SEQ ID:454.

Another function of VGAM119 is therefore inhibition of LOC116166 (Accession XM_{—}007651). Accordingly, utilities of VGAM119 include diagnosis, prevention and treatment of diseases and clinical conditions associated with LOC116166. LOC123855 (Accession XM_{—}063871) is another VGAM119 host target gene. LOC123855 BINDING SITE is HOST TARGET binding site found in the 3′ untranslated region of mRNA encoded by LOC123855, corresponding to a HOST TARGET binding site such as BINDING SITE I, BINDING SITE II or BINDING SITE III. Table 2 illustrates the complementarity of the nucleotide sequences of LOC123855 BINDING SITE, designated SEQ ID:3033, to the nucleotide sequence of VGAM119 RNA, herein designated VGAM RNA, also designated SEQ ID:454.

Another function of VGAM119 is therefore inhibition of LOC123855 (Accession XM_{—}063871). Accordingly, utilities of VGAM119 include diagnosis, prevention and treatment of diseases and clinical conditions associated with LOC123855. LOC146818 (Accession XM_{—}085598) is another VGAM119 host target gene. LOC146818 BINDING SITE is HOST TARGET binding site found in the 5′ untranslated region of mRNA encoded by LOC146818, corresponding to a HOST TARGET binding site such as BINDING SITE I, BINDING SITE II or BINDING SITE III. Table 2 illustrates the complementarity of the nucleotide sequences of LOC146818 BINDING SITE, designated SEQ ID:3100, to the nucleotide sequence of VGAM119 RNA, herein designated VGAM RNA, also designated SEQ ID:454.

Another function of VGAM119 is therefore inhibition of LOC146818 (Accession XM_{—}085598). Accordingly, utilities of VGAM119 include diagnosis, prevention and treatment of diseases and clinical conditions associated with LOC146818. LOC152190 (Accession XM_{—}045692) is another VGAM119 host target gene. LOC152190 BINDING SITE is HOST TARGET binding site found in the 5′ untranslated region of mRNA encoded by LOC152190, corresponding to a HOST TARGET binding site such as BINDING SITE I, BINDING SITE II or BINDING SITE III. Table 2 illustrates the complementarity of the nucleotide sequences of LOC152190 BINDING SITE, designated SEQ ID:2864, to the nucleotide sequence of VGAM119 RNA, herein designated VGAM RNA, also designated SEQ ID:454.

Another function of VGAM119 is therefore inhibition of LOC152190 (Accession XM_{—}045692). Accordingly, utilities of VGAM119 include diagnosis, prevention and treatment of diseases and clinical conditions associated with LOC152190. LOC153077 (Accession XM_{—}098307) is another VGAM119 host target gene. LOC153077 BINDING SITE is HOST TARGET binding site found in the 3′ untranslated region of mRNA encoded by LOC153077, corresponding to a HOST TARGET binding site such as BINDING SITE I, BINDING SITE II or BINDING SITE III. Table 2 illustrates the complementarity of the nucleotide sequences of LOC153077 BINDING SITE, designated SEQ ID:3366, to the nucleotide sequence of VGAM119 RNA, herein designated VGAM RNA, also designated SEQ ID:454.

Another function of VGAM119 is therefore inhibition of LOC153077 (Accession XM_{—}098307). Accordingly, utilities of VGAM119 include diagnosis, prevention and treatment of diseases and clinical conditions associated with LOC153077. LOC158382 (Accession XM_{—}098931) is another VGAM119 host target gene. LOC158382 BINDING SITE is HOST TARGET binding site found in the 3′ untranslated region of mRNA encoded by LOC158382, corresponding to a HOST TARGET binding site such as BINDING SITE I, BINDING SITE II or BINDING SITE III. Table 2 illustrates the complementarity of the nucleotide sequences of LOC158382 BINDING SITE, designated SEQ ID:3388, to the nucleotide sequence of VGAM119 RNA, herein designated VGAM RNA, also designated SEQ ID:454.

Another function of VGAM119 is therefore inhibition of LOC158382 (Accession XM_{—}098931). Accordingly, utilities of VGAM119 include diagnosis, prevention and treatment of diseases and clinical conditions associated with LOC158382. LOC158476 (Accession XM_{—}098955) is another VGAM119 host target gene. LOC158476 BINDING SITE is HOST TARGET binding site found in the 3′ untranslated region of mRNA encoded by LOC158476, corresponding to a HOST TARGET binding site such as BINDING SITE I, BINDING SITE II or BINDING SITE III. Table 2 illustrates the complementarity of the nucleotide sequences of LOC158476 BINDING SITE, designated SEQ ID:3391, to the nucleotide sequence of VGAM119 RNA, herein designated VGAM RNA, also designated SEQ ID:454.

Another function of VGAM119 is therefore inhibition of LOC158476 (Accession XM_{—}098955). Accordingly, utilities of VGAM119 include diagnosis, prevention and treatment of diseases and clinical conditions associated with LOC158476. LOC164397 (Accession XM_{—}092780) is another VGAM119 host target gene. LOC164397 BINDING SITE is HOST TARGET binding site found in the 5′ untranslated region of mRNA encoded by LOC164397, corresponding to a HOST TARGET binding site such as BINDING SITE I, BINDING SITE II or BINDING SITE III. Table 2 illustrates the complementarity of the nucleotide sequences of LOC164397 BINDING SITE, designated SEQ ID:3249, to the nucleotide sequence of VGAM119 RNA, herein designated VGAM RNA, also designated SEQ ID:454.

Another function of VGAM119 is therefore inhibition of LOC164397 (Accession XM_{—}092780). Accordingly, utilities of VGAM119 include diagnosis, prevention and treatment of diseases and clinical conditions associated with LOC164397. LOC196510 (Accession XM_{—}113738) is another VGAM119 host target gene. LOC196510 BINDING SITE is HOST TARGET binding site found in the 3′ untranslated region of mRNA encoded by LOC196510, corresponding to a HOST TARGET binding site such as BINDING SITE I, BINDING SITE II or BINDING SITE III. Table 2 illustrates the complementarity of the nucleotide sequences of LOC196510 BINDING SITE, designated SEQ ID:3416, to the nucleotide sequence of VGAM119 RNA, herein designated VGAM RNA, also designated SEQ ID:454.

Another function of VGAM119 is therefore inhibition of LOC196510 (Accession XM_{—}113738). Accordingly, utilities of VGAM119 include diagnosis, prevention and treatment of diseases and clinical conditions associated with LOC196510. LOC200220 (Accession XM_{—}114157) is another VGAM119 host target gene. LOC200220 BINDING SITE is HOST TARGET binding site found in the 3′ untranslated region of mRNA encoded by LOC200220, corresponding to a HOST TARGET binding site such as BINDING SITE I, BINDING SITE II or BINDING SITE III. Table 2 illustrates the complementarity of the nucleotide sequences of LOC200220 BINDING SITE, designated SEQ ID:3445, to the nucleotide sequence of VGAM119 RNA, herein designated VGAM RNA, also designated SEQ ID:454.

Another function of VGAM119 is therefore inhibition of LOC200220 (Accession XM_{—}114157). Accordingly, utilities of VGAM119 include diagnosis, prevention and treatment of diseases and clinical conditions associated with LOC200220. LOC201477 (Accession XM_{—}117098) is another VGAM119 host target gene. LOC201477 BINDING SITE is HOST TARGET binding site found in the 5′ untranslated region of mRNA encoded by LOC201477, corresponding to a HOST TARGET binding site such as BINDING SITE I, BINDING SITE II or BINDING SITE III. Table 2 illustrates the complementarity of the nucleotide sequences of LOC201477 BINDING SITE, designated SEQ ID:3472, to the nucleotide sequence of VGAM119 RNA, herein designated VGAM RNA, also designated SEQ ID:454.

Another function of VGAM119 is therefore inhibition of LOC201477 (Accession XM_{—}117098). Accordingly, utilities of VGAM119 include diagnosis, prevention and treatment of diseases and clinical conditions associated with LOC201477. LOC201696 (Accession XM_{—}032269) is another VGAM119 host target gene. LOC201696 BINDING SITE is HOST TARGET binding site found in the 5′ untranslated region of mRNA encoded by LOC201696, corresponding to a HOST TARGET binding site such as BINDING SITE I, BINDING SITE II or BINDING SITE III. Table 2 illustrates the complementarity of the nucleotide sequences of LOC201696 BINDING SITE, designated SEQ ID:2650, to the nucleotide sequence of VGAM119 RNA, herein designated VGAM RNA, also designated SEQ ID:454.

Another function of VGAM119 is therefore inhibition of LOC201696 (Accession XM_{—}032269). Accordingly, utilities of VGAM119 include diagnosis, prevention and treatment of diseases and clinical conditions associated with LOC201696. LOC220469 (Accession XM_{—}084334) is another VGAM119 host target gene. LOC220469 BINDING SITE is HOST TARGET binding site found in the 3′ untranslated region of mRNA encoded by LOC220469, corresponding to a HOST TARGET binding site such as BINDING SITE I, BINDING SITE II or BINDING SITE III. Table 2 illustrates the complementarity of the nucleotide sequences of LOC220469 BINDING SITE, designated SEQ ID:3049, to the nucleotide sequence of VGAM119 RNA, herein designated VGAM RNA, also designated SEQ ID:454.

Another function of VGAM119 is therefore inhibition of LOC220469 (Accession XM_{—}084334). Accordingly, utilities of VGAM119 include diagnosis, prevention and treatment of diseases and clinical conditions associated with LOC220469. LOC221272 (Accession XM_{—}168050) is another VGAM119 host target gene. LOC221272 BINDING SITE is HOST TARGET binding site found in the 3′ untranslated region of mRNA encoded by LOC221272, corresponding to a HOST TARGET binding site such as BINDING SITE I, BINDING SITE II or BINDING SITE III. Table 2 illustrates the complementarity of the nucleotide sequences of LOC221272 BINDING SITE, designated SEQ ID:3615, to the nucleotide sequence of VGAM119 RNA, herein designated VGAM RNA, also designated SEQ ID:454.

Another function of VGAM119 is therefore inhibition of LOC221272 (Accession XM_{—}168050). Accordingly, utilities of VGAM119 include diagnosis, prevention and treatment of diseases and clinical conditions associated with LOC221272. LOC222166 (Accession XM_{—}168425) is another VGAM119 host target gene. LOC222166 BINDING SITE is HOST TARGET binding site found in the 3′ untranslated region of mRNA encoded by LOC222166, corresponding to a HOST TARGET binding site such as BINDING SITE I, BINDING SITE II or BINDING SITE III. Table 2 illustrates the complementarity of the nucleotide sequences of LOC222166 BINDING SITE, designated SEQ ID:3636, to the nucleotide sequence of VGAM119 RNA, herein designated VGAM RNA, also designated SEQ ID:454.

Another function of VGAM119 is therefore inhibition of LOC222166 (Accession XM_{—}168425). Accordingly, utilities of VGAM119 include diagnosis, prevention and treatment of diseases and clinical conditions associated with LOC222166. LOC253220 (Accession XM_{—}172831) is another VGAM119 host target gene. LOC253220 BINDING SITE is HOST TARGET binding site found in the 5′ untranslated region of mRNA encoded by LOC253220, corresponding to a HOST TARGET binding site such as BINDING SITE I, BINDING SITE II or BINDING SITE III. Table 2 illustrates the complementarity of the nucleotide sequences of LOC253220 BINDING SITE, designated SEQ ID:3706, to the nucleotide sequence of VGAM119 RNA, herein designated VGAM RNA, also designated SEQ ID:454.

Another function of VGAM119 is therefore inhibition of LOC253220 (Accession XM_{—}172831). Accordingly, utilities of VGAM119 include diagnosis, prevention and treatment of diseases and clinical conditions associated with LOC253220. LOC51275 (Accession NM_{—}016534) is another VGAM119 host target gene. LOC51275 BINDING SITE is HOST TARGET binding site found in the 5′ untranslated region of mRNA encoded by LOC51275, corresponding to a HOST TARGET binding site such as BINDING SITE I, BINDING SITE II or BINDING SITE III. Table 2 illustrates the complementarity of the nucleotide sequences of LOC51275 BINDING SITE, designated SEQ ID:1694, to the nucleotide sequence of VGAM119 RNA, herein designated VGAM RNA, also designated SEQ ID:454.

Another function of VGAM119 is therefore inhibition of LOC51275 (Accession NM_{—}016534). Accordingly, utilities of VGAM119 include diagnosis, prevention and treatment of diseases and clinical conditions associated with LOC51275. LOC54505 (Accession XM_{—}042110) is another VGAM119 host target gene. LOC54505 BINDING SITE is HOST TARGET binding site found in the 5′ untranslated region of mRNA encoded by LOC54505, corresponding to a HOST TARGET binding site such as BINDING SITE I, BINDING SITE II or BINDING SITE III. Table 2 illustrates the complementarity of the nucleotide sequences of LOC54505 BINDING SITE, designated SEQ ID:2800, to the nucleotide sequence of VGAM119 RNA, herein designated VGAM RNA, also designated SEQ ID:454.

Another function of VGAM119 is therefore inhibition of LOC54505 (Accession XM_{—}042110). Accordingly, utilities of VGAM119 include diagnosis, prevention and treatment of diseases and clinical conditions associated with LOC54505. LOC90844 (Accession XM_{—}034434) is another VGAM119 host target gene. LOC90844 BINDING SITE is HOST TARGET binding site found in the 3′ untranslated region of mRNA encoded by LOC90844, corresponding to a HOST TARGET binding site such as BINDING SITE I, BINDING SITE II or BINDING SITE III. Table 2 illustrates the complementarity of the nucleotide sequences of LOC90844 BINDING SITE, designated SEQ ID:2682, to the nucleotide sequence of VGAM119 RNA, herein designated VGAM RNA, also designated SEQ ID:454.

Another function of VGAM119 is therefore inhibition of LOC90844 (Accession XM_{—}034434). Accordingly, utilities of VGAM119 include diagnosis, prevention and treatment of diseases and clinical conditions associated with LOC90844. LOC92697 (Accession XM_{—}046715) is another VGAM119 host target gene. LOC92697 BINDING SITE is HOST TARGET binding site found in the 5′ untranslated region of mRNA encoded by LOC92697, corresponding to a HOST TARGET binding site such as BINDING SITE I, BINDING SITE II or BINDING SITE III. Table 2 illustrates the complementarity of the nucleotide sequences of LOC92697 BINDING SITE, designated SEQ ID:2882, to the nucleotide sequence of VGAM119 RNA, herein designated VGAM RNA, also designated SEQ ID:454.

Another function of VGAM119 is therefore inhibition of LOC92697 (Accession XM_{—}046715). Accordingly, utilities of VGAM119 include diagnosis, prevention and treatment of diseases and clinical conditions associated with LOC92697. FIG. 1 further provides a conceptual description of a novel bioinformatically detected viral gene of the present invention, referred to here as Viral Genomic Address Messenger 120 (VGAM120) viral gene, which modulates expression of respective host target genes thereof, the function and utility of which host target genes is known in the art.

VGAM120 is a novel bioinformatically detected regulatory, non protein coding, viral micro RNA (miRNA) gene. The method by which VGAM120 was detected is described hereinabove with reference to FIGS. 1-8.

VGAM120 gene, herein designated VGAM GENE, is a viral gene contained in the genome of Vaccinia Virus. VGAM120 host target gene, herein designated VGAM HOST TARGET GENE, is a human gene contained in the human genome.

VGAM120 gene encodes a VGAM120 precursor RNA, herein designated VGAM PRECURSOR RNA. Similar to other miRNA genes, and unlike most ordinary genes, VGAM120 precursor RNA does not encode a protein. A nucleotide sequence identical or highly similar to the nucleotide sequence of VGAM120 precursor RNA is designated SEQ ID:106, and is provided hereinbelow with reference to the sequence listing part. Nucleotide sequence SEQ ID: 106 is located at position 50332 relative to the genome of Vaccinia Virus.

VGAM120 precursor RNA folds onto itself, forming VGAM120 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM120 folded precursor RNA into VGAM120 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM120 RNA is designated SEQ ID:455, and is provided hereinbelow with reference to the sequence listing part.

VGAM120 host target gene, herein designated VGAM HOST TARGET GENE, encodes a corresponding messenger RNA, VGAM120 host target RNA, herein designated VGAM HOST TARGET RNA. VGAM120 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM120 RNA, herein designated VGAM RNA, binds complementarily to one or more host target binding sites located in untranslated regions of VGAM120 host target RNA, herein designated VGAM HOST TARGET RNA. This complementary binding is due to the fact that the nucleotide sequence of VGAM120 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM120 RNA, herein designated VGAM RNA, may have a different number of host target binding sites in untranslated regions of a VGAM120 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM120 RNA, herein designated VGAM RNA, to host target binding sites on VGAM120 host target RNA, herein designated VGAM HOST TARGET RNA, such as BINDING SITE I, BINDING SITE II and BINDING SITE III, inhibits translation of VGAM120 host target RNA into VGAM120 host target protein, herein designated VGAM HOST TARGET PROTEIN. VGAM host target protein is therefore outlined by a broken line.

It is appreciated that VGAM120 host target gene, herein designated VGAM HOST TARGET GENE, in fact represents a plurality of VGAM120 host target genes. The mRNA of each one of this plurality of VGAM120 host target genes comprises one or more host target binding sites, each having a nucleotide sequence which is at least partly complementary to VGAM120 RNA, herein designated VGAM RNA, and which when bound by VGAM120 RNA causes inhibition of translation of respective one or more VGAM120 host target proteins.

It is further appreciated by one skilled in the art that the mode of translational inhibition illustrated by FIG. 1 with specific reference to translational inhibition exerted by VGAM120 gene, herein designated VGAM GENE, on one or more VGAM120 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM120 is inhibition of expression of host target genes, as part of a novel viral mechanism of attacking a host. Accordingly, utilities of VGAM120 include diagnosis, prevention and treatment of viral infection by Vaccinia Virus. Specific functions, and accordingly utilities, of VGAM120 correlate with, and may be deduced from, the identity of the host target genes which VGAM120 binds and inhibits, and the function of these host target genes, as elaborated hereinbelow.

Nucleotide sequences of the VGAM120 precursor RNA, herein designated VGAM PRECURSOR RNA, and of the ‘diced’ VGAM120 RNA, herein designated VGAM RNA, and a schematic representation of the secondary folding of VGAM120 folded precursor RNA, herein designated VGAM FOLDED PRECURSOR RNA, of VGAM120 are further described hereinbelow with reference to Table 1.

Nucleotide sequences of host target binding sites, such as BINDING SITE-I, BINDING SITE-II and BINDING SITE-III of FIG. 1, found on VGAM120 host target RNA, and schematic representation of the complementarity of each of these host target binding sites to VGAM120 RNA, herein designated VGAM RNA, are described hereinbelow with reference to Table 2.

As mentioned hereinabove with reference to FIG. 1, a function of VGAM120 gene, herein designated VGAM is inhibition of expression of VGAM120 target genes. It is appreciated that specific functions, and accordingly utilities, of VGAM120 correlate with, and may be deduced from, the identity of the target genes which VGAM120 binds and inhibits, and the function of these target genes, as elaborated hereinbelow.

Kinesin Family Member 5C (KIF5C, Accession NM_{—}004522) is a VGAM120 host target gene. KIF5C BINDING SITE is HOST TARGET binding site found in the 3′ untranslated region of mRNA encoded by KIF5C, corresponding to a HOST TARGET binding site such as BINDING SITE I, BINDING SITE II or BINDING SITE III. Table 2 illustrates the complementarity of the nucleotide sequences of KIF5C BINDING SITE, designated SEQ ID:1122, to the nucleotide sequence of VGAM120 RNA, herein designated VGAM RNA, also designated SEQ ID:455.

A function of VGAM120 is therefore inhibition of Kinesin Family Member 5C (KIF5C, Accession NM_{—}004522). Accordingly, utilities of VGAM120 include diagnosis, prevention and treatment of diseases and clinical conditions associated with KIF5C. DKFZP564M182 (Accession XM_{—}085525) is another VGAM120 host target gene. DKFZP564M182 BINDING SITE is HOST TARGET binding site found in the 3′ untranslated region of mRNA encoded by DKFZP564M182, corresponding to a HOST TARGET binding site such as BINDING SITE I, BINDING SITE II or BINDING SITE III. Table 2 illustrates the complementarity of the nucleotide sequences of DKFZP564M182 BINDING SITE, designated SEQ ID:3097, to the nucleotide sequence of VGAM120 RNA, herein designated VGAM RNA, also designated SEQ ID:455.

Another function of VGAM120 is therefore inhibition of DKFZP564M182 (Accession XM_{—}085525). Accordingly, utilities of VGAM120 include diagnosis, prevention and treatment of diseases and clinical conditions associated with DKFZP564M182. KIAA0852 (Accession NM_{—}014941) is another VGAM120 host target gene. KIAA0852 BINDING SITE is HOST TARGET binding site found in the 5′ untranslated region of mRNA encoded by KIAA0852, corresponding to a HOST TARGET binding site such as BINDING SITE I, BINDING SITE II or BINDING SITE III. Table 2 illustrates the complementarity of the nucleotide sequences of KIAA0852 BINDING SITE, designated SEQ ID:1594, to the nucleotide sequence of VGAM120 RNA, herein designated VGAM RNA, also designated SEQ ID:455.

Another function of VGAM120 is therefore inhibition of KIAA0852 (Accession NM_{—}014941). Accordingly, utilities of VGAM120 include diagnosis, prevention and treatment of diseases and clinical conditions associated with KIAA0852. PRO0097 (Accession NM_{—}014114) is another VGAM120 host target gene. PRO0097 BINDING SITE is HOST TARGET binding site found in the 5′ untranslated region of mRNA encoded by PRO0097, corresponding to a HOST TARGET binding site such as BINDING SITE I, BINDING SITE II or BINDING SITE III. Table 2 illustrates the complementarity of the nucleotide sequences of PRO0097 BINDING SITE, designated SEQ ID: 1472, to the nucleotide sequence of VGAM120 RNA, herein designated VGAM RNA, also designated SEQ ID:455.

Another function of VGAM120 is therefore inhibition of PRO0097 (Accession NM_{—}014114). Accordingly, utilities of VGAM120 include diagnosis, prevention and treatment of diseases and clinical conditions associated with PRO0097. LOC222161 (Accession XM_{—}166596) is another VGAM120 host target gene. LOC222161 BINDING SITE is HOST TARGET binding site found in the 5′ untranslated region of mRNA encoded by LOC222161, corresponding to a HOST TARGET binding site such as BINDING SITE I, BINDING SITE II or BINDING SITE III. Table 2 illustrates the complementarity of the nucleotide sequences of LOC222161 BINDING SITE, designated SEQ ID:3578, to the nucleotide sequence of VGAM120 RNA, herein designated VGAM RNA, also designated SEQ ID:455.

Another function of VGAM120 is therefore inhibition of LOC222161 (Accession XM_{—}166596). Accordingly, utilities of VGAM120 include diagnosis, prevention and treatment of diseases and clinical conditions associated with LOC222161. LOC255811 (Accession XM_{—}170625) is another VGAM120 host target gene. LOC255811 BINDING SITE is HOST TARGET binding site found in the 5′ untranslated region of mRNA encoded by LOC255811, corresponding to a HOST TARGET binding site such as BINDING SITE I, BINDING SITE II or BINDING SITE III. Table 2 illustrates the complementarity of the nucleotide sequences of LOC255811 BINDING SITE, designated SEQ ID:3662, to the nucleotide sequence of VGAM120 RNA, herein designated VGAM RNA, also designated SEQ ID:455.

Another function of VGAM120 is therefore inhibition of LOC255811 (Accession XM_{—}170625). Accordingly, utilities of VGAM120 include diagnosis, prevention and treatment of diseases and clinical conditions associated with LOC255811. FIG. 1 further provides a conceptual description of a novel bioinformatically detected viral gene of the present invention, referred to here as Viral Genomic Address Messenger 121 (VGAM121) viral gene, which modulates expression of respective host target genes thereof, the function and utility of which host target genes is known in the art.

VGAM121 is a novel bioinformatically detected regulatory, non protein coding, viral micro RNA (miRNA) gene. The method by which VGAM121 was detected is described hereinabove with reference to FIGS. 1-8.

VGAM121 gene, herein designated VGAM GENE, is a viral gene contained in the genome of Vaccinia Virus. VGAM121 host target gene, herein designated VGAM HOST TARGET GENE, is a human gene contained in the human genome.

VGAM121 gene encodes a VGAM121 precursor RNA, herein designated VGAM PRECURSOR RNA. Similar to other miRNA genes, and unlike most ordinary genes, VGAM121 precursor RNA does not encode a protein. A nucleotide sequence identical or highly similar to the nucleotide sequence of VGAM121 precursor RNA is designated SEQ ID:107, and is provided hereinbelow with reference to the sequence listing part. Nucleotide sequence SEQ ID: 107 is located at position 47859 relative to the genome of Vaccinia Virus.

VGAM121 precursor RNA folds onto itself, forming VGAM121 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM121 folded precursor RNA into VGAM121 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM121 RNA is designated SEQ ID:456, and is provided hereinbelow with reference to the sequence listing part.

VGAM121 host target gene, herein designated VGAM HOST TARGET GENE, encodes a corresponding messenger RNA, VGAM121 host target RNA, herein designated VGAM HOST TARGET RNA. VGAM121 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM121 RNA, herein designated VGAM RNA, binds complementarily to one or more host target binding sites located in untranslated regions of VGAM121 host target RNA, herein designated VGAM HOST TARGET RNA. This complementary binding is due to the fact that the nucleotide sequence of VGAM121 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM121 RNA, herein designated VGAM RNA, may have a different number of host target binding sites in untranslated regions of a VGAM121 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM121 RNA, herein designated VGAM RNA, to host target binding sites on VGAM121 host target RNA, herein designated VGAM HOST TARGET RNA, such as BINDING SITE I, BINDING SITE II and BINDING SITE III, inhibits translation of VGAM121 host target RNA into VGAM121 host target protein, herein designated VGAM HOST TARGET PROTEIN. VGAM host target protein is therefore outlined by a broken line.

It is appreciated that VGAM121 host target gene, herein designated VGAM HOST TARGET GENE, in fact represents a plurality of VGAM121 host target genes. The mRNA of each one of this plurality of VGAM121 host target genes comprises one or more host target binding sites, each having a nucleotide sequence which is at least partly complementary to VGAM121 RNA, herein designated VGAM RNA, and which when bound by VGAM121 RNA causes inhibition of translation of respective one or more VGAM121 host target proteins.

It is further appreciated by one skilled in the art that the mode of translational inhibition illustrated by FIG. 1 with specific reference to translational inhibition exerted by VGAM121 gene, herein designated VGAM GENE, on one or more VGAM121 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM121 is inhibition of expression of host target genes, as part of a novel viral mechanism of attacking a host. Accordingly, utilities of VGAM121 include diagnosis, prevention and treatment of viral infection by Vaccinia Virus. Specific functions, and accordingly utilities, of VGAM121 correlate with, and may be deduced from, the identity of the host target genes which VGAM121 binds and inhibits, and the function of these host target genes, as elaborated hereinbelow.

Nucleotide sequences of the VGAM121 precursor RNA, herein designated VGAM PRECURSOR RNA, and of the ‘diced’ VGAM121 RNA, herein designated VGAM RNA, and a schematic representation of the secondary folding of VGAM121 folded precursor RNA, herein designated VGAM FOLDED PRECURSOR RNA, of VGAM121 are further described hereinbelow with reference to Table 1.

Nucleotide sequences of host target binding sites, such as BINDING SITE-I, BINDING SITE-II and BINDING SITE-III of FIG. 1, found on VGAM121 host target RNA, and schematic representation of the complementarity of each of these host target binding sites to VGAM121 RNA, herein designated VGAM RNA, are described hereinbelow with reference to Table 2.

As mentioned hereinabove with reference to FIG. 1, a function of VGAM121 gene, herein designated VGAM is inhibition of expression of VGAM121 target genes. It is appreciated that specific functions, and accordingly utilities, of VGAM121 correlate with, and may be deduced from, the identity of the target genes which VGAM121 binds and inhibits, and the function of these target genes, as elaborated hereinbelow.

Dihydropyrimidine Dehydrogenase (DPYD, Accession XM_{—}017469) is a VGAM121 host target gene. DPYD BINDING SITE is HOST TARGET binding site found in the 3′ untranslated region of mRNA encoded by DPYD, corresponding to a HOST TARGET binding site such as BINDING SITE I, BINDING SITE II or BINDING SITE III. Table 2 illustrates the complementarity of the nucleotide sequences of DPYD BINDING SITE, designated SEQ ID:2569, to the nucleotide sequence of VGAM121 RNA, herein designated VGAM RNA, also designated SEQ ID:456.

A function of VGAM121 is therefore inhibition of Dihydropyrimidine Dehydrogenase (DPYD, Accession XM_{—}017469). Accordingly, utilities of VGAM121 include diagnosis, prevention and treatment of diseases and clinical conditions associated with DPYD. KIAA1240 (Accession XM_{—}039676) is another VGAM121 host target gene. KIAA1240 BINDING SITE is HOST TARGET binding site found in the 3′ untranslated region of mRNA encoded by KIAA1240, corresponding to a HOST TARGET binding site such as BINDING SITE I, BINDING SITE II or BINDING SITE III. Table 2 illustrates the complementarity of the nucleotide sequences of KIAA1240 BINDING SITE, designated SEQ ID:2761, to the nucleotide sequence of VGAM121 RNA, herein designated VGAM RNA, also designated SEQ ID:456.

Another function of VGAM121 is therefore inhibition of KIAA1240 (Accession XM_{—}039676). Accordingly, utilities of VGAM121 include diagnosis, prevention and treatment of diseases and clinical conditions associated with KIAA1240. KIAA1843 (Accession XM_{—}030838) is another VGAM121 host target gene. KIAA1843 BINDING SITE is HOST TARGET binding site found in the 3′ untranslated region of mRNA encoded by KIAA1843, corresponding to a HOST TARGET binding site such as BINDING SITE I, BINDING SITE II or BINDING SITE III. Table 2 illustrates the complementarity of the nucleotide sequences of KIAA1843 BINDING SITE, designated SEQ ID:2624, to the nucleotide sequence of VGAM121 RNA, herein designated VGAM RNA, also designated SEQ ID:456.

Another function of VGAM121 is therefore inhibition of KIAA1843 (Accession XM_{—}030838). Accordingly, utilities of VGAM121 include diagnosis, prevention and treatment of diseases and clinical conditions associated with KIAA1843. LOC122786 (Accession XM_{—}058660) is another VGAM121 host target gene. LOC122786 BINDING SITE is HOST TARGET binding site found in the 3′ untranslated region of mRNA encoded by LOC122786, corresponding to a HOST TARGET binding site such as BINDING SITE I, BINDING SITE II or BINDING SITE III. Table 2 illustrates the complementarity of the nucleotide sequences of LOC122786 BINDING SITE, designated SEQ ID:2995, to the nucleotide sequence of VGAM121 RNA, herein designated VGAM RNA, also designated SEQ ID:456.

Another function of VGAM121 is therefore inhibition of LOC122786 (Accession XM_{—}058660). Accordingly, utilities of VGAM121 include diagnosis, prevention and treatment of diseases and clinical conditions associated with LOC122786. FIG. 1 further provides a conceptual description of a novel bioinformatically detected viral gene of the present invention, referred to here as Viral Genomic Address Messenger 122 (VGAM122) viral gene, which modulates expression of respective host target genes thereof, the function and utility of which host target genes is known in the art.

VGAM122 is a novel bioinformatically detected regulatory, non protein coding, viral micro RNA (miRNA) gene. The method by which VGAM122 was detected is described hereinabove with reference to FIGS. 1-8.

VGAM122 gene, herein designated VGAM GENE, is a viral gene contained in the genome of Vaccinia Virus. VGAM122 host target gene, herein designated VGAM HOST TARGET GENE, is a human gene contained in the human genome.

VGAM122 gene encodes a VGAM122 precursor RNA, herein designated VGAM PRECURSOR RNA. Similar to other miRNA genes, and unlike most ordinary genes, VGAM122 precursor RNA does not encode a protein. A nucleotide sequence identical or highly similar to the nucleotide sequence of VGAM122 precursor RNA is designated SEQ ID:108, and is provided hereinbelow with reference to the sequence listing part. Nucleotide sequence SEQ ID: 108 is located at position 50837 relative to the genome of Vaccinia Virus.

VGAM122 precursor RNA folds onto itself, forming VGAM122 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM122 folded precursor RNA into VGAM122 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM122 RNA is designated SEQ ID:457, and is provided hereinbelow with reference to the sequence listing part.

VGAM122 host target gene, herein designated VGAM HOST TARGET GENE, encodes a corresponding messenger RNA, VGAM122 host target RNA, herein designated VGAM HOST TARGET RNA. VGAM122 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM122 RNA, herein designated VGAM RNA, binds complementarily to one or more host target binding sites located in untranslated regions of VGAM122 host target RNA, herein designated VGAM HOST TARGET RNA. This complementary binding is due to the fact that the nucleotide sequence of VGAM122 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM122 RNA, herein designated VGAM RNA, may have a different number of host target binding sites in untranslated regions of a VGAM122 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM122 RNA, herein designated VGAM RNA, to host target binding sites on VGAM122 host target RNA, herein designated VGAM HOST TARGET RNA, such as BINDING SITE I, BINDING SITE II and BINDING SITE III, inhibits translation of VGAM122 host target RNA into VGAM122 host target protein, herein designated VGAM HOST TARGET PROTEIN. VGAM host target protein is therefore outlined by a broken line.

It is appreciated that VGAM122 host target gene, herein designated VGAM HOST TARGET GENE, in fact represents a plurality of VGAM122 host target genes. The mRNA of each one of this plurality of VGAM122 host target genes comprises one or more host target binding sites, each having a nucleotide sequence which is at least partly complementary to VGAM122 RNA, herein designated VGAM RNA, and which when bound by VGAM122 RNA causes inhibition of translation of respective one or more VGAM122 host target proteins.

It is further appreciated by one skilled in the art that the mode of translational inhibition illustrated by FIG. 1 with specific reference to translational inhibition exerted by VGAM122 gene, herein designated VGAM GENE, on one or more VGAM122 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM122 is inhibition of expression of host target genes, as part of a novel viral mechanism of attacking a host. Accordingly, utilities of VGAM122 include diagnosis, prevention and treatment of viral infection by Vaccinia Virus. Specific functions, and accordingly utilities, of VGAM122 correlate with, and may be deduced from, the identity of the host target genes which VGAM122 binds and inhibits, and the function of these host target genes, as elaborated hereinbelow.

Nucleotide sequences of the VGAM122 precursor RNA, herein designated VGAM PRECURSOR RNA, and of the ‘diced’ VGAM122 RNA, herein designated VGAM RNA, and a schematic representation of the secondary folding of VGAM122 folded precursor RNA, herein designated VGAM FOLDED PRECURSOR RNA, of VGAM122 are further described hereinbelow with reference to Table 1.

Nucleotide sequences of host target binding sites, such as BINDING SITE-I, BINDING SITE-II and BINDING SITE-III of FIG. 1, found on VGAM122 host target RNA, and schematic representation of the complementarity of each of these host target binding sites to VGAM122 RNA, herein designated VGAM RNA, are described hereinbelow with reference to Table 2.

As mentioned hereinabove with reference to FIG. 1, a function of VGAM122 gene, herein designated VGAM is inhibition of expression of VGAM122 target genes. It is appreciated that specific functions, and accordingly utilities, of VGAM122 correlate with, and may be deduced from, the identity of the target genes which VGAM122 binds and inhibits, and the function of these target genes, as elaborated hereinbelow.

Fibroblast Growth Factor 12 (FGF12, Accession NM_{—}021032) is a VGAM122 host target gene. FGF12 BINDING SITE is HOST TARGET binding site found in the 3′ untranslated region of mRNA encoded by FGF12, corresponding to a HOST TARGET binding site such as BINDING SITE I, BINDING SITE II or BINDING SITE III. Table 2 illustrates the complementarity of the nucleotide sequences of FGF12 BINDING SITE, designated SEQ ID:1935, to the nucleotide sequence of VGAM122 RNA, herein designated VGAM RNA, also designated SEQ ID:457.

A function of VGAM122 is therefore inhibition of Fibroblast Growth Factor 12 (FGF12, Accession NM_{—}021032), a gene which probably involved in nervous system development and function. Accordingly, utilities of VGAM122 include diagnosis, prevention and treatment of diseases and clinical conditions associated with FGF12. The function of FGF12 has been established by previous studies. Fibroblast growth factors comprise a family of related polypeptides with broad mitogenic and cell survival activities. Smallwood et al. (1996) noted that FGF1 (OMIM Ref. No. 131220), or acidic FGF, and FGF2 (OMIM Ref. No. 134920), or basic FGF, were the first 2 family members to be identified, purified, and sequenced, and are widely expressed as a potent mitogen for a variety of cell types. FGF3 (OMIM Ref. No. 164950) is a common target for activation by the mouse mammary tumor virus. The genes encoding FGF4 (OMIM Ref. No. 164980), FGF5 (OMIM Ref. No. 165190), and FGF6 (OMIM Ref. No. 134921) have transforming activity when introduced into NIH 3T3 cells. FGF7 (OMIM Ref. No. 148180), FGF8 (OMIM Ref. No. 600483), and FGF9 (OMIM Ref. No. 600921) are mitogens for keratinocytes, mammary carcinoma cells, and astrocytes, respectively. Several FGFs have been found to have additional bioactivities that were not evident during their initial identification. The 9 known FGFs are between 150 and 268 amino acid residues in length and share a conserved central region of approximately 140 amino acids. FGF signaling is generally assumed to occur by activation of transmembrane tyrosine kinase receptors. Four FGF receptors, FGFR1 (OMIM Ref. No. 136350) through FGFR4 (OMIM Ref. No. 134935), had been identified, and activating or inactivating receptor mutations have been described for a subset of these genes in both mice and humans. Smallwood et al. (1996) identified and characterized 4 additional members of the FGF family, which they referred to as fibroblast growth factor homologous factors (FHFs). The genes were identified by a combination of random cDNA sequencing, database searches, and degenerate PCR. Pairwise comparisons between the 4 FHFs show between 58% and 71% amino acid sequence identity, but each FHF shows less than 30% identity when compared with other FGFs. Like FGF1 and FGF2, the FHFs lack a classic signal sequence and contain clusters of basic residues that can act as nuclear localization signals. In transiently transfected 293 cells, FHF1 accumulates in the nucleus and is not secreted. By Southern blot hybridization of genomic DNA from rodent/human hybrid cell lines containing individual human chromosomes, Smallwood et al. (1996) demonstrated that the human FHF1 (also symbolized FGF12), FHF2 (OMIM Ref. No. 300070), FHF3 (OMIM Ref. No. 601514), and FHF4 (OMIM Ref. No. 601515) genes are located on chromosomes 3, X, 17, and 13, respectively. They found that a sequence tagged site (STS) that encompassed 1 exon of FHF3 was derived from human chromosome 17 and mapped near the BRCA1 gene (OMIM Ref. No. 113705), which is located at 17q21. The chromosomal locations of Fhf1, Fhf2, and Fhf4 in the mouse were determined using an interspecific mapping panel. Fhf1 mapped to the proximal region of mouse chromosome 16, 1.6 cM distal to somatostatin (OMIM Ref. No. 182450) and 5.1 cM proximal to ApoD (OMIM Ref. No. 107740). Fhf2 mapped to the mouse X chromosome and did not recombine with the CD40 ligand gene (OMIM Ref. No. 300386) in 168 mice typed, suggesting that the 2 loci are within 1.8 cM of each other. Fhf4 mapped to the distal region of chromosome 14 and did not recombine with Rap2a (OMIM Ref. No. 179540) in 142 mice typed in common. The Fhf3 gene was not mapped with the backcross panel because it failed to demonstrate an informative RFLP when tested with 14 restriction enzymes. The proximity of the human FHF3 gene to BRCA1 suggested to Smallwood et al. (1996) that the mouse homolog resides on chromosome 11 in the region that is syntenic with the BRCA1 region of human chromosome 17. From the location of the Fhf1 gene in the mouse one can suggest that the human gene is located on 3q28. Smallwood et al. (1996) showed that FHFs are expressed principally in the nervous system and are therefore likely to play a role in nervous system development and/or function. Liu and Chiu (1997) mapped the FGF12 gene to 3q29-qter by fluorescence in situ hybridization.

Full details of the abovementioned studies are described in the following publications, the disclosure of which are hereby incorporated by reference:
Liu, Y.; Chiu, I.-M.: Assignment of FGF12, the human FGF homologous factor 1 gene, to chromosome 3q29-3qter by fluorescence in situ hybridization. Cytogenet. Cell Genet. 78: 48-49, 1997.; and
Smallwood, P. M.; Munoz-Sanjuan, I.; Tong, P.; Macke, J. P.; Hendry, S. H. C.; Gilbert, D. J.; Copeland, N. G.; Jenkins, N. A.; Nathans, J.: Fibroblast growth factor (FGF) homologous f.

Further studies establishing the function and utilities of FGF12 are found in John Hopkins OMIM database record ID 601513, and in sited publications numbered 64 and 2039 listed in the bibliography section hereinbelow, which are also hereby incorporated by reference. Fibrinogen, Gamma Polypeptide (FGG, Accession NM_{—}021870) is another VGAM122 host target gene. FGG BINDING SITE is HOST TARGET binding site found in the 3′ untranslated region of mRNA encoded by FGG, corresponding to a HOST TARGET binding site such as BINDING SITE I, BINDING SITE II or BINDING SITE III. Table 2 illustrates the complementarity of the nucleotide sequences of FGG BINDING SITE, designated SEQ ID:1965, to the nucleotide sequence of VGAM122 RNA, herein designated VGAM RNA, also designated SEQ ID:457.

Another function of VGAM122 is therefore inhibition of Fibrinogen, Gamma Polypeptide (FGG, Accession NM_{—}021870). Accordingly, utilities of VGAM122 include diagnosis, prevention and treatment of diseases and clinical conditions associated with FGG. NEBL (Accession NM_{—}006393) is another VGAM122 host target gene. NEBL BINDING SITE is HOST TARGET binding site found in the 3′ untranslated region of mRNA encoded by NEBL, corresponding to a HOST TARGET binding site such as BINDING SITE I, BINDING SITE II or BINDING SITE III. Table 2 illustrates the complementarity of the nucleotide sequences of NEBL BINDING SITE, designated SEQ ID:1295, to the nucleotide sequence of VGAM122 RNA, herein designated VGAM RNA, also designated SEQ ID:457.

Another function of VGAM122 is therefore inhibition of NEBL (Accession NM_{—}006393). Accordingly, utilities of VGAM122 include diagnosis, prevention and treatment of diseases and clinical conditions associated with NEBL. Ribulose-5-phosphate-3-epimerase (RPE, Accession XM_{—}030834) is another VGAM122 host target gene. RPE BINDING SITE is HOST TARGET binding site found in the 3′ untranslated region of mRNA encoded by RPE, corresponding to a HOST TARGET binding site such as BINDING SITE I, BINDING SITE II or BINDING SITE III. Table 2 illustrates the complementarity of the nucleotide sequences of RPE BINDING SITE, designated SEQ ID:2623, to the nucleotide sequence of VGAM122 RNA, herein designated VGAM RNA, also designated SEQ ID:457.

Another function of VGAM122 is therefore inhibition of Ribulose-5-phosphate-3-epimerase (RPE, Accession XM_{—}030834). Accordingly, utilities of VGAM122 include diagnosis, prevention and treatment of diseases and clinical conditions associated with RPE. TRHDE (Accession NM_{—}013381) is another VGAM122 host target gene. TRHDE BINDING SITE is HOST TARGET binding site found in the 3′ untranslated region of mRNA encoded by TRHDE, corresponding to a HOST TARGET binding site such as BINDING SITE I, BINDING SITE II or BINDING SITE III. Table 2 illustrates the complementarity of the nucleotide sequences of TRHDE BINDING SITE, designated SEQ ID: 1446, to the nucleotide sequence of VGAM122 RNA, herein designated VGAM RNA, also designated SEQ ID:457.

Another function of VGAM122 is therefore inhibition of TRHDE (Accession NM_{—}013381). Accordingly, utilities of VGAM122 include diagnosis, prevention and treatment of diseases and clinical conditions associated with TRHDE. BCMP1 (Accession NM_{—}031442) is another VGAM122 host target gene. BCMP1 BINDING SITE is HOST TARGET binding site found in the 3′ untranslated region of mRNA encoded by BCMP1, corresponding to a HOST TARGET binding site such as BINDING SITE I, BINDING SITE II or BINDING SITE III. Table 2 illustrates the complementarity of the nucleotide sequences of BCMP1 BINDING SITE, designated SEQ ID:2197, to the nucleotide sequence of VGAM122 RNA, herein designated VGAM RNA, also designated SEQ ID:457.

Another function of VGAM122 is therefore inhibition of BCMP1 (Accession NM_{—}031442). Accordingly, utilities of VGAM122 include diagnosis, prevention and treatment of diseases and clinical conditions associated with BCMP1. DKFZP434A0225 (Accession XM_{—}168185) is another VGAM122 host target gene. DKFZP434A0225 BINDING SITE is HOST TARGET binding site found in the 3′ untranslated region of mRNA encoded by DKFZP434A0225, corresponding to a HOST TARGET binding site such as BINDING SITE I, BINDING SITE II or BINDING SITE III. Table 2 illustrates the complementarity of the nucleotide sequences of DKFZP434A0225 BINDING SITE, designated SEQ ID:3624, to the nucleotide sequence of VGAM122 RNA, herein designated VGAM RNA, also designated SEQ ID:457.

Another function of VGAM122 is therefore inhibition of DKFZP434A0225 (Accession XM_{—}168185). Accordingly, utilities of VGAM122 include diagnosis, prevention and treatment of diseases and clinical conditions associated with DKFZP434A0225. FLJ12899 (Accession NM_{—}024594) is another VGAM122 host target gene. FLJ12899 BINDING SITE is HOST TARGET binding site found in the 3′ untranslated region of mRNA encoded by FLJ12899, corresponding to a HOST TARGET binding site such as BINDING SITE I, BINDING SITE II or BINDING SITE III. Table 2 illustrates the complementarity of the nucleotide sequences of FLJ12899 BINDING SITE, designated SEQ ID:2069, to the nucleotide sequence of VGAM122 RNA, herein designated VGAM RNA, also designated SEQ ID:457.

Another function of VGAM122 is therefore inhibition of FLJ12899 (Accession NM_{—}024594). Accordingly, utilities of VGAM122 include diagnosis, prevention and treatment of diseases and clinical conditions associated with FLJ12899. Preimplantation Protein 3 (PREI3, Accession XM_{—}038960) is another VGAM122 host target gene. PREI3 BINDING SITE is HOST TARGET binding site found in the 3′ untranslated region of mRNA encoded by PREI3, corresponding to a HOST TARGET binding site such as BINDING SITE I, BINDING SITE II or BINDING SITE III. Table 2 illustrates the complementarity of the nucleotide sequences of PREI3 BINDING SITE, designated SEQ ID:2747, to the nucleotide sequence of VGAM122 RNA, herein designated VGAM RNA, also designated SEQ ID:457.

Another function of VGAM122 is therefore inhibition of Preimplantation Protein 3 (PREI3, Accession XM_{—}038960). Accordingly, utilities of VGAM122 include diagnosis, prevention and treatment of diseases and clinical conditions associated with PREI3. PC4 and SFRS1 Interacting Protein 2 (PSIP2, Accession NM_{—}033222) is another VGAM122 host target gene. PSIP2 BINDING SITE is HOST TARGET binding site found in the 3′ untranslated region of mRNA encoded by PSIP2, corresponding to a HOST TARGET binding site such as BINDING SITE I, BINDING SITE II or BINDING SITE III. Table 2 illustrates the complementarity of the nucleotide sequences of PSIP2 BINDING SITE, designated SEQ ID:2323, to the nucleotide sequence of VGAM122 RNA, herein designated VGAM RNA, also designated SEQ ID:457.

Another function of VGAM122 is therefore inhibition of PC4 and SFRS1 Interacting Protein 2 (PSIP2, Accession NM_{—}033222). Accordingly, utilities of VGAM122 include diagnosis, prevention and treatment of diseases and clinical conditions associated with PSIP2. LOC220963 (Accession XM_{—}166145) is another VGAM122 host target gene. LOC220963 BINDING SITE is HOST TARGET binding site found in the 3′ untranslated region of mRNA encoded by LOC220963, corresponding to a HOST TARGET binding site such as BINDING SITE I, BINDING SITE II or BINDING SITE III. Table 2 illustrates the complementarity of the nucleotide sequences of LOC220963 BINDING SITE, designated SEQ ID:3526, to the nucleotide sequence of VGAM122 RNA, herein designated VGAM RNA, also designated SEQ ID:457.

Another function of VGAM122 is therefore inhibition of LOC220963 (Accession XM_{—}166145). Accordingly, utilities of VGAM122 include diagnosis, prevention and treatment of diseases and clinical conditions associated with LOC220963. FIG. 1 further provides a conceptual description of a novel bioinformatically detected viral gene of the present invention, referred to here as Viral Genomic Address Messenger 123 (VGAM123) viral gene, which modulates expression of respective host target genes thereof, the function and utility of which host target genes is known in the art.

VGAM123 is a novel bioinformatically detected regulatory, non protein coding, viral micro RNA (miRNA) gene. The method by which VGAM123 was detected is described hereinabove with reference to FIGS. 1-8.

VGAM123 gene, herein designated VGAM GENE, is a viral gene contained in the genome of Vaccinia Virus. VGAM123 host target gene, herein designated VGAM HOST TARGET GENE, is a human gene contained in the human genome.

VGAM123 gene encodes a VGAM123 precursor RNA, herein designated VGAM PRECURSOR RNA. Similar to other miRNA genes, and unlike most ordinary genes, VGAM123 precursor RNA does not encode a protein. A nucleotide sequence identical or highly similar to the nucleotide sequence of VGAM123 precursor RNA is designated SEQ ID:109, and is provided hereinbelow with reference to the sequence listing part. Nucleotide sequence SEQ ID: 109 is located at position 52454 relative to the genome of Vaccinia Virus.

VGAM123 precursor RNA folds onto itself, forming VGAM123 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM123 folded precursor RNA into VGAM123 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM123 RNA is designated SEQ ID:458, and is provided hereinbelow with reference to the sequence listing part.

VGAM123 host target gene, herein designated VGAM HOST TARGET GENE, encodes a corresponding messenger RNA, VGAM123 host target RNA, herein designated VGAM HOST TARGET RNA. VGAM123 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM123 RNA, herein designated VGAM RNA, binds complementarily to one or more host target binding sites located in untranslated regions of VGAM123 host target RNA, herein designated VGAM HOST TARGET RNA. This complementary binding is due to the fact that the nucleotide sequence of VGAM123 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM123 RNA, herein designated VGAM RNA, may have a different number of host target binding sites in untranslated regions of a VGAM123 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM123 RNA, herein designated VGAM RNA, to host target binding sites on VGAM123 host target RNA, herein designated VGAM HOST TARGET RNA, such as BINDING SITE I, BINDING SITE II and BINDING SITE III, inhibits translation of VGAM123 host target RNA into VGAM123 host target protein, herein designated VGAM HOST TARGET PROTEIN. VGAM host target protein is therefore outlined by a broken line.

It is appreciated that VGAM123 host target gene, herein designated VGAM HOST TARGET GENE, in fact represents a plurality of VGAM123 host target genes. The mRNA of each one of this plurality of VGAM123 host target genes comprises one or more host target binding sites, each having a nucleotide sequence which is at least partly complementary to VGAM123 RNA, herein designated VGAM RNA, and which when bound by VGAM123 RNA causes inhibition of translation of respective one or more VGAM123 host target proteins.

It is further appreciated by one skilled in the art that the mode of translational inhibition illustrated by FIG. 1 with specific reference to translational inhibition exerted by VGAM123 gene, herein designated VGAM GENE, on one or more VGAM123 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM123 is inhibition of expression of host target genes, as part of a novel viral mechanism of attacking a host. Accordingly, utilities of VGAM123 include diagnosis, prevention and treatment of viral infection by Vaccinia Virus. Specific functions, and accordingly utilities, of VGAM123 correlate with, and may be deduced from, the identity of the host target genes which VGAM123 binds and inhibits, and the function of these host target genes, as elaborated hereinbelow.

Nucleotide sequences of the VGAM123 precursor RNA, herein designated VGAM PRECURSOR RNA, and of the ‘diced’ VGAM123 RNA, herein designated VGAM RNA, and a schematic representation of the secondary folding of VGAM123 folded precursor RNA, herein designated VGAM FOLDED PRECURSOR RNA, of VGAM123 are further described hereinbelow with reference to Table 1.

Nucleotide sequences of host target binding sites, such as BINDING SITE-I, BINDING SITE-II and BINDING SITE-III of FIG. 1, found on VGAM123 host target RNA, and schematic representation of the complementarity of each of these host target binding sites to VGAM123 RNA, herein designated VGAM RNA, are described hereinbelow with reference to Table 2.

As mentioned hereinabove with reference to FIG. 1, a function of VGAM123 gene, herein designated VGAM is inhibition of expression of VGAM123 target genes. It is appreciated that specific functions, and accordingly utilities, of VGAM123 correlate with, and may be deduced from, the identity of the target genes which VGAM123 binds and inhibits, and the function of these target genes, as elaborated hereinbelow.

LOC143098 (Accession XM_{—}084421) is a VGAM123 host target gene. LOC143098 BINDING SITE is HOST TARGET binding site found in the 5′ untranslated region of mRNA encoded by LOC143098, corresponding to a HOST TARGET binding site such as BINDING SITE I, BINDING SITE II or BINDING SITE III. Table 2 illustrates the complementarity of the nucleotide sequences of LOC143098 BINDING SITE, designated SEQ ID:3051, to the nucleotide sequence of VGAM123 RNA, herein designated VGAM RNA, also designated SEQ ID:458.

A function of VGAM123 is therefore inhibition of LOC143098 (Accession XM_{—}084421). Accordingly, utilities of VGAM123 include diagnosis, prevention and treatment of diseases and clinical conditions associated with LOC143098. FIG. 1 further provides a conceptual description of a novel bioinformatically detected viral gene of the present invention, referred to here as Viral Genomic Address Messenger 124 (VGAM124) viral gene, which modulates expression of respective host target genes thereof, the function and utility of which host target genes is known in the art.

VGAM124 is a novel bioinformatically detected regulatory, non protein coding, viral micro RNA (miRNA) gene. The method by which VGAM124 was detected is described hereinabove with reference to FIGS. 1-8.

VGAM124 gene, herein designated VGAM GENE, is a viral gene contained in the genome of Vaccinia Virus. VGAM124 host target gene, herein designated VGAM HOST TARGET GENE, is a human gene contained in the human genome.

VGAM124 gene encodes a VGAM124 precursor RNA, herein designated VGAM PRECURSOR RNA. Similar to other miRNA genes, and unlike most ordinary genes, VGAM124 precursor RNA does not encode a protein. A nucleotide sequence identical or highly similar to the nucleotide sequence of VGAM124 precursor RNA is designated SEQ ID:110, and is provided hereinbelow with reference to the sequence listing part. Nucleotide sequence SEQ ID:110 is located at position 52549 relative to the genome of Vaccinia Virus.

VGAM124 precursor RNA folds onto itself, forming VGAM124 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM124 folded precursor RNA into VGAM124 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM124 RNA is designated SEQ ID:459, and is provided hereinbelow with reference to the sequence listing part.

VGAM124 host target gene, herein designated VGAM HOST TARGET GENE, encodes a corresponding messenger RNA, VGAM124 host target RNA, herein designated VGAM HOST TARGET RNA. VGAM124 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM124 RNA, herein designated VGAM RNA, binds complementarily to one or more host target binding sites located in untranslated regions of VGAM124 host target RNA, herein designated VGAM HOST TARGET RNA. This complementary binding is due to the fact that the nucleotide sequence of VGAM124 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM124 RNA, herein designated VGAM RNA, may have a different number of host target binding sites in untranslated regions of a VGAM124 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM124 RNA, herein designated VGAM RNA, to host target binding sites on VGAM124 host target RNA, herein designated VGAM HOST TARGET RNA, such as BINDING SITE I, BINDING SITE II and BINDING SITE III, inhibits translation of VGAM124 host target RNA into VGAM124 host target protein, herein designated VGAM HOST TARGET PROTEIN. VGAM host target protein is therefore outlined by a broken line.

It is appreciated that VGAM124 host target gene, herein designated VGAM HOST TARGET GENE, in fact represents a plurality of VGAM124 host target genes. The mRNA of each one of this plurality of VGAM124 host target genes comprises one or more host target binding sites, each having a nucleotide sequence which is at least partly complementary to VGAM124 RNA, herein designated VGAM RNA, and which when bound by VGAM124 RNA causes inhibition of translation of respective one or more VGAM124 host target proteins.

It is further appreciated by one skilled in the art that the mode of translational inhibition illustrated by FIG. 1 with specific reference to translational inhibition exerted by VGAM124 gene, herein designated VGAM GENE, on one or more VGAM124 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM124 is inhibition of expression of host target genes, as part of a novel viral mechanism of attacking a host. Accordingly, utilities of VGAM124 include diagnosis, prevention and treatment of viral infection by Vaccinia Virus. Specific functions, and accordingly utilities, of VGAM124 correlate with, and may be deduced from, the identity of the host target genes which VGAM124 binds and inhibits, and the function of these host target genes, as elaborated hereinbelow.

Nucleotide sequences of the VGAM124 precursor RNA, herein designated VGAM PRECURSOR RNA, and of the ‘diced’ VGAM124 RNA, herein designated VGAM RNA, and a schematic representation of the secondary folding of VGAM124 folded precursor RNA, herein designated VGAM FOLDED PRECURSOR RNA, of VGAM124 are further described hereinbelow with reference to Table 1.

Nucleotide sequences of host target binding sites, such as BINDING SITE-I, BINDING SITE-II and BINDING SITE-III of FIG. 1, found on VGAM124 host target RNA, and schematic representation of the complementarity of each of these host target binding sites to VGAM124 RNA, herein designated VGAM RNA, are described hereinbelow with reference to Table 2.

As mentioned hereinabove with reference to FIG. 1, a function of VGAM124 gene, herein designated VGAM is inhibition of expression of VGAM124 target genes. It is appreciated that specific functions, and accordingly utilities, of VGAM124 correlate with, and may be deduced from, the identity of the target genes which VGAM124 binds and inhibits, and the function of these target genes, as elaborated hereinbelow.

Calbindin 2, 29 kDa (calretinin) (CALB2, Accession NM_{—}001740) is a VGAM124 host target gene. CALB2 BINDING SITE is HOST TARGET binding site found in the 3′ untranslated region of mRNA encoded by CALB2, corresponding to a HOST TARGET binding site such as BINDING SITE I, BINDING SITE II or BINDING SITE III. Table 2 illustrates the complementarity of the nucleotide sequences of CALB2 BINDING SITE, designated SEQ ID:853, to the nucleotide sequence of VGAM124 RNA, herein designated VGAM RNA, also designated SEQ ID:459.

A function of VGAM124 is therefore inhibition of Calbindin 2, 29 kDa (calretinin) (CALB2, Accession NM_{—}001740), a gene which plays a major role at the network level in cerebellar physiology. Accordingly, utilities of VGAM124 include diagnosis, prevention and treatment of diseases and clinical conditions associated with CALB2. The function of CALB2 has been established by previous studies. Using a genomic fragment containing exon 2 of the brain calcium-binding protein, calbindin 29 kD, in the study of human/rodent somatic cell hybrids, Parmentier et al. (1989) assigned the gene to chromosome 16. Chen et al. (1991) mapped the CALB2 gene and 11 others to the long arm of chromosome 16 by the use of 14 mouse/human hybrid cell lines and the fragile site FRA16B. The CALB2 gene was found to be in the distal portion of band 16q22.1, just proximal to HP (OMIM Ref. No. 140100) and just distal to NMOR1 (DIA4; 125860). By in situ hybridization, Parmentier et al. (1991) mapped the CALB2 gene, called by them calretinin, to 16q22-q23. Schiffmann et al. (1999) showed that Calb2-deficient mice were impaired in tests of motor coordination. Impairment in intracellular calcium concentration in the Calb2-deficient Purkinje cells was supported by the high calcium saturation of calbindin-D28K (CALB1; 114050) in these cells. The firing behavior of Purkinje cells was severely affected in alert null mice, with alterations of simple spike firing rate, complex spike duration, and simple spike pause. In contrast, transmission at parallel fiber- or climbing fiber-Purkinje cell synapses was unaltered in slices, indicating that marked modifications of firing behavior in vivo can be undetectable in slice. The results were interpreted as indicating that CALB2 plays a major role at the network level in cerebellar physiology.

Full details of the abovementioned studies are described in the following publications, the disclosure of which are hereby incorporated by reference:
Schiffmann, S. N.; Cheron, G.; Lohof, A.; d'Alcantara, P.; Meyer, M.; Parmentier, M.; Schurmans, S.: Impaired motor coordination and Purkinje cell excitability in mice lacking calretinin. Proc. Nat. Acad. Sci. 96: 5257-5262, 1999.; and
Chen, L. Z.; Harris, P. C.; Apostolou, S.; Baker, E.; Holman, K.; Lane, S. A.; Nancarrow, J. K.; Whitmore, S. A.; Stallings, R. L.; Hildebrand, C. E.; Richards, R. I.; Sutherland, G. R.

Further studies establishing the function and utilities of CALB2 are found in John Hopkins OMIM database record ID 114051, and in sited publications numbered 2743-406 listed in the bibliography section hereinbelow, which are also hereby incorporated by reference. TNF Receptor-associated Factor 5 (TRAF5, Accession NM_{—}004619) is another VGAM124 host target gene. TRAF5 BINDING SITE is HOST TARGET binding site found in the 3′ untranslated region of mRNA encoded by TRAF5, corresponding to a HOST TARGET binding site such as BINDING SITE I, BINDING SITE II or BINDING SITE III. Table 2 illustrates the complementarity of the nucleotide sequences of TRAF5 BINDING SITE, designated SEQ ID:1133, to the nucleotide sequence of VGAM124 RNA, herein designated VGAM RNA, also designated SEQ ID:459.

Another function of VGAM124 is therefore inhibition of TNF Receptor-associated Factor 5 (TRAF5, Accession NM_{—}004619), a gene which Member of a family of proteins that interact with TNF receptors; binds the lymphotoxin beta receptor (LTBR). Accordingly, utilities of VGAM124 include diagnosis, prevention and treatment of diseases and clinical conditions associated with TRAF5. The function of TRAF5 has been established by previous studies. Tumor necrosis factor (TNF; 191160) receptor-associated factors (TRAFs) are signal transducers for members of the TNF receptor super family (see OMIM Ref. No. 191190). TRAF proteins are composed of an N-terminal cysteine/histidine-rich region containing zinc RING and/or zinc finger motifs, a coiled coil (leucine zipper) motif, and a homologous region in the C terminus that defines the TRAF family, the TRAF domain. The TRAF domain is involved in self-association and receptor binding. By degenerative oligonucleotide PCR amplification, Nakano et al. (1996) identified TRAF5 in the mouse and showed that its specifically interacts with the lymphotoxin-beta receptor (OMIM Ref. No. 600979) and activates the transcription factor NF-kappa-B (see OMIM Ref. No. 164011). Nakano et al. (1997) cloned the human TRAF homolog by cross hybridization with mouse TRAF5 cDNA. Their human cDNA of 2,894 bp has a 557-amino acid open reading frame that exhibits 77.5 and 80% identity to mouse TRAF5 at the nucleotide and amino acid levels, respectively. Northern blot analysis revealed that human TRAF5 mRNA is expressed in all visceral organs. Western blotting revealed that the human protein is abundantly expressed in a human follicular dendritic cell line, and to a lesser degree in several tumor cell lines. By in vitro binding, immunoprecipitation, immunoblot, and yeast 2-hybrid analyses, Aizawa et al. (1997) showed that TRAF2 (OMIM Ref. No. 601895) and TRAF5 interact with overlapping but distinct sequences in the C-terminal region of CD30 (OMIM Ref. No. 153243) and mediate the activation of NFKB. By interspecific backcross mapping, Nakano et al. (1997) showed that Traf5 is located in the distal region of mouse chromosome 1, which shares homology with human 1q. Fluorescence in situ hybridization confirmed the regional localization of human TRAF5 to chromosome 1q32. To investigate the functional role of Traf5 in vivo, Nakano et al. (1999) generated Traf5-deficient mice by gene targeting. They found that Traf5 −/− B lymphocytes show defects in proliferation and up-regulation of various surface molecules, including CD23 (OMIM Ref. No. 151445), CD54 (OMIM Ref. No. 147840), CD80 (OMIM Ref. No. 112203), CD86 (OMIM Ref. No. 601020), and FAS (OMIM Ref. No. 134637) in response to CD40 (OMIM Ref. No. 109535) stimulation. Moreover, in vitro Ig production by Traf5 −/− lymphocytes stimulated with anti-CD40 plus IL4 (OMIM Ref. No. 147780) was reduced substantially. CD27-mediated costimulatory signal also was impaired in Traf5 −/− T lymphocytes. Collectively, these results demonstrated that Traf5 is involved in CD40- and CD27-mediated signaling.

Full details of the abovementioned studies are described in the following publications, the disclosure of which are hereby incorporated by reference:
Nakano, H.; Sakon, S.; Koseki, H.; Takemori, T.; Tada, K.; Matsumoto, M.; Munechika, E.; Sakai, T.; Shirasawa, T.; Akiba, H; Kobata, T.; Santee, S. M.; Ware, C. F.; Renner, P. D.; Taniguchi, M.; Yagita, H.; Okumura, K.: Targeted disruption of Traf5 gene causes defects in CD40- and CD27-mediated lymphocyte activation. Proc. Nat. Acad. Sci. 96: 9803-9808, 1999.; and
Nakano, H.; Shindo, M.; Yamada, K.; Yoshida, M. C.; Santee, S. M.; Ware, C. F.; Jenkins, N. A.; Gilbert, D. J.; Yagita, H.; Copeland, N. G.; Okumura, K.: Human TNF receptor-associated.

Further studies establishing the function and utilities of TRAF5 are found in John Hopkins OMIM database record ID 602356, and in sited publications numbered 2665-2019 listed in the bibliography section hereinbelow, which are also hereby incorporated by reference. Chromosome 15 Open Reading Frame 5 (C15orf5, Accession NM_{—}030944) is another VGAM124 host target gene. C15orf5 BINDING SITE is HOST TARGET binding site found in the 3′ untranslated region of mRNA encoded by C15orf5, corresponding to a HOST TARGET binding site such as BINDING SITE I, BINDING SITE II or BINDING SITE III. Table 2 illustrates the complementarity of the nucleotide sequences of C15orf5 BINDING SITE, designated SEQ ID:2173, to the nucleotide sequence of VGAM124 RNA, herein designated VGAM RNA, also designated SEQ ID:459.

Another function of VGAM124 is therefore inhibition of Chromosome 15 Open Reading Frame 5 (C15orf5, Accession NM_{—}030944). Accordingly, utilities of VGAM124 include diagnosis, prevention and treatment of diseases and clinical conditions associated with C15orf5. DKFZp761K1423 (Accession NM_{—}018422) is another VGAM124 host target gene. DKFZp761K1423 BINDING SITE is HOST TARGET binding site found in the 3′ untranslated region of mRNA encoded by DKFZp761K1423, corresponding to a HOST TARGET binding site such as BINDING SITE I, BINDING SITE II or BINDING SITE III. Table 2 illustrates the complementarity of the nucleotide sequences of DKFZp761K1423 BINDING SITE, designated SEQ ID: 1826, to the nucleotide sequence of VGAM124 RNA, herein designated VGAM RNA, also designated SEQ ID:459.

Another function of VGAM124 is therefore inhibition of DKFZp761K1423 (Accession NM_{—}018422). Accordingly, utilities of VGAM124 include diagnosis, prevention and treatment of diseases and clinical conditions associated with DKFZp761K1423. FLJ12892 (Accession XM_{—}042173) is another VGAM124 host target gene. FLJ12892 BINDING SITE is HOST TARGET binding site found in the 5′ untranslated region of mRNA encoded by FLJ12892, corresponding to a HOST TARGET binding site such as BINDING SITE I, BINDING SITE II or BINDING SITE III. Table 2 illustrates the complementarity of the nucleotide sequences of FLJ12892 BINDING SITE, designated SEQ ID:2801, to the nucleotide sequence of VGAM124 RNA, herein designated VGAM RNA, also designated SEQ ID:459.

Another function of VGAM124 is therefore inhibition of FLJ12892 (Accession XM_{—}042173). Accordingly, utilities of VGAM124 include diagnosis, prevention and treatment of diseases and clinical conditions associated with FLJ12892. MGC15437 (Accession NM_{—}032873) is another VGAM124 host target gene. MGC15437 BINDING SITE is HOST TARGET binding site found in the 3′ untranslated region of mRNA encoded by MGC15437, corresponding to a HOST TARGET binding site such as BINDING SITE I, BINDING SITE II or BINDING SITE III. Table 2 illustrates the complementarity of the nucleotide sequences of MGC15437 BINDING SITE, designated SEQ ID:2292, to the nucleotide sequence of VGAM124 RNA, herein designated VGAM RNA, also designated SEQ ID:459.

Another function of VGAM124 is therefore inhibition of MGC15437 (Accession NM_{—}032873). Accordingly, utilities of VGAM124 include diagnosis, prevention and treatment of diseases and clinical conditions associated with MGC15437. MGC27016 (Accession NM_{—}144979) is another VGAM124 host target gene. MGC27016 BINDING SITE is HOST TARGET binding site found in the 3′ untranslated region of mRNA encoded by MGC27016, corresponding to a HOST TARGET binding site such as BINDING SITE I, BINDING SITE II or BINDING SITE III. Table 2 illustrates the complementarity of the nucleotide sequences of MGC27016 BINDING SITE, designated SEQ ID:2503, to the nucleotide sequence of VGAM124 RNA, herein designated VGAM RNA, also designated SEQ ID:459.

Another function of VGAM124 is therefore inhibition of MGC27016 (Accession NM_{—}144979). Accordingly, utilities of VGAM124 include diagnosis, prevention and treatment of diseases and clinical conditions associated with MGC27016. LOC154089 (Accession XM_{—}087846) is another VGAM124 host target gene. LOC154089 BINDING SITE is HOST TARGET binding site found in the 3′ untranslated region of mRNA encoded by LOC154089, corresponding to a HOST TARGET binding site such as BINDING SITE I, BINDING SITE II or BINDING SITE III. Table 2 illustrates the complementarity of the nucleotide sequences of LOC154089 BINDING SITE, designated SEQ ID:3187, to the nucleotide sequence of VGAM124 RNA, herein designated VGAM RNA, also designated SEQ ID:459.

Another function of VGAM124 is therefore inhibition of LOC154089 (Accession XM_{—}087846). Accordingly, utilities of VGAM124 include diagnosis, prevention and treatment of diseases and clinical conditions associated with LOC154089. LOC159053 (Accession XM_{—}099021) is another VGAM124 host target gene. LOC159053 BINDING SITE is HOST TARGET binding site found in the 3′ untranslated region of mRNA encoded by LOC159053, corresponding to a HOST TARGET binding site such as BINDING SITE I, BINDING SITE II or BINDING SITE III. Table 2 illustrates the complementarity of the nucleotide sequences of LOC159053 BINDING SITE, designated SEQ ID:3395, to the nucleotide sequence of VGAM124 RNA, herein designated VGAM RNA, also designated SEQ ID:459.

Another function of VGAM124 is therefore inhibition of LOC159053 (Accession XM_{—}099021). Accordingly, utilities of VGAM124 include diagnosis, prevention and treatment of diseases and clinical conditions associated with LOC159053. FIG. 1 further provides a conceptual description of a novel bioinformatically detected viral gene of the present invention, referred to here as Viral Genomic Address Messenger 125 (VGAM125) viral gene, which modulates expression of respective host target genes thereof, the function and utility of which host target genes is known in the art.

VGAM125 is a novel bioinformatically detected regulatory, non protein coding, viral micro RNA (miRNA) gene. The method by which VGAM125 was detected is described hereinabove with reference to FIGS. 1-8.

VGAM125 gene, herein designated VGAM GENE, is a viral gene contained in the genome of Vaccinia Virus. VGAM125 host target gene, herein designated VGAM HOST TARGET GENE, is a human gene contained in the human genome.

VGAM125 gene encodes a VGAM125 precursor RNA, herein designated VGAM PRECURSOR RNA. Similar to other miRNA genes, and unlike most ordinary genes, VGAM125 precursor RNA does not encode a protein. A nucleotide sequence identical or highly similar to the nucleotide sequence of VGAM125 precursor RNA is designated SEQ ID: 111, and is provided hereinbelow with reference to the sequence listing part. Nucleotide sequence SEQ ID: 111 is located at position 55395 relative to the genome of Vaccinia Virus.

VGAM125 precursor RNA folds onto itself, forming VGAM125 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM125 folded precursor RNA into VGAM125 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM125 RNA is designated SEQ ID:460, and is provided hereinbelow with reference to the sequence listing part.

VGAM125 host target gene, herein designated VGAM HOST TARGET GENE, encodes a corresponding messenger RNA, VGAM125 host target RNA, herein designated VGAM HOST TARGET RNA. VGAM125 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM125 RNA, herein designated VGAM RNA, binds complementarily to one or more host target binding sites located in untranslated regions of VGAM125 host target RNA, herein designated VGAM HOST TARGET RNA. This complementary binding is due to the fact that the nucleotide sequence of VGAM125 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM125 RNA, herein designated VGAM RNA, may have a different number of host target binding sites in untranslated regions of a VGAM125 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM125 RNA, herein designated VGAM RNA, to host target binding sites on VGAM125 host target RNA, herein designated VGAM HOST TARGET RNA, such as BINDING SITE I, BINDING SITE II and BINDING SITE III, inhibits translation of VGAM125 host target RNA into VGAM125 host target protein, herein designated VGAM HOST TARGET PROTEIN. VGAM host target protein is therefore outlined by a broken line.

It is appreciated that VGAM125 host target gene, herein designated VGAM HOST TARGET GENE, in fact represents a plurality of VGAM125 host target genes. The mRNA of each one of this plurality of VGAM125 host target genes comprises one or more host target binding sites, each having a nucleotide sequence which is at least partly complementary to VGAM125 RNA, herein designated VGAM RNA, and which when bound by VGAM125 RNA causes inhibition of translation of respective one or more VGAM125 host target proteins.

It is further appreciated by one skilled in the art that the mode of translational inhibition illustrated by FIG. 1 with specific reference to translational inhibition exerted by VGAM125 gene, herein designated VGAM GENE, on one or more VGAM125 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM125 is inhibition of expression of host target genes, as part of a novel viral mechanism of attacking a host. Accordingly, utilities of VGAM125 include diagnosis, prevention and treatment of viral infection by Vaccinia Virus. Specific functions, and accordingly utilities, of VGAM125 correlate with, and may be deduced from, the identity of the host target genes which VGAM125 binds and inhibits, and the function of these host target genes, as elaborated hereinbelow.

Nucleotide sequences of the VGAM125 precursor RNA, herein designated VGAM PRECURSOR RNA, and of the ‘diced’ VGAM125 RNA, herein designated VGAM RNA, and a schematic representation of the secondary folding of VGAM125 folded precursor RNA, herein designated VGAM FOLDED PRECURSOR RNA, of VGAM125 are further described hereinbelow with reference to Table 1.

Nucleotide sequences of host target binding sites, such as BINDING SITE-I, BINDING SITE-II and BINDING SITE-III of FIG. 1, found on VGAM125 host target RNA, and schematic representation of the complementarity of each of these host target binding sites to VGAM125 RNA, herein designated VGAM RNA, are described hereinbelow with reference to Table 2.

As mentioned hereinabove with reference to FIG. 1, a function of VGAM125 gene, herein designated VGAM is inhibition of expression of VGAM125 target genes. It is appreciated that specific functions, and accordingly utilities, of VGAM125 correlate with, and may be deduced from, the identity of the target genes which VGAM125 binds and inhibits, and the function of these target genes, as elaborated hereinbelow.

Xenotropic and Polytropic Retrovirus Receptor (XPR1, Accession NM_{—}004736) is a VGAM125 host target gene. XPR1 BINDING SITE is HOST TARGET binding site found in the 3′ untranslated region of mRNA encoded by XPR1, corresponding to a HOST TARGET binding site such as BINDING SITE I, BINDING SITE II or BINDING SITE III. Table 2 illustrates the complementarity of the nucleotide sequences of XPR1 BINDING SITE, designated SEQ ID:1152, to the nucleotide sequence of VGAM125 RNA, herein designated VGAM RNA, also designated SEQ ID:460.

A function of VGAM125 is therefore inhibition of Xenotropic and Polytropic Retrovirus Receptor (XPR1, Accession NM_{—}004736), a gene which is a putative G protein-coupled receptor and a target for xenotropic and polytropic murine leukemia retroviruses. Accordingly, utilities of VGAM125 include diagnosis, prevention and treatment of diseases and clinical conditions associated with XPR1. The function of XPR1 has been established by previous studies. There are 4 classes of murine leukemia virus (MLV): xenotropic (X), ecotropic (E), amphotropic (A), and polytropic (P). X— and E-MLV cannot exogenously infect mouse cells and are inherited as part of the mouse genome. While X-MLV can infect other mammalian species but not cells from laboratory mice, A- (see OMIM Ref. No. SLC20A2; 158378) and P-MLV can infect mouse and other species. See Levy (1999) for a review of MLVs. By cloning a human T-lymphocyte cDNA library into a retroviral vector, transducing the library into naturally X-MLV-resistant mouse fibroblasts, and PCR amplification, Tailor et al. (1999) isolated a cDNA encoding XPR1. Expression of XPR1 in mouse and hamster MLV-resistant fibroblasts rendered the cells susceptible to both X— and P-MLV. The deduced 696-amino acid XPR1 protein contains 8 or 9 potential membrane-spanning regions, 7 potential N-glycosylation sites, and 7 dileucines that may stimulate endocytosis via clathrin-coated pits. Northern blot analysis detected a 4.5-kb XPR1 transcript in all tissues tested, with highest expression in pancreas, kidney, placenta, hematopoietic tissues, and heart, and lowest expression in skeletal muscle. Expression of XPR1 was greater in fetal liver than adult liver. A 9.5-kb XPR1 transcript was also detected in all tissues tested except liver and bone marrow.

Full details of the abovementioned studies are described in the following publications, the disclosure of which are hereby incorporated by reference:
Levy, J. A.: Xenotropism: the elusive viral receptor finally uncovered. Proc. Nat. Acad. Sci. 96: 802-804, 1999.; and
Tailor, C. S.; Nouri, A.; Lee, C. G.; Kozak, C.; Kabat, D.: Cloning and characterization of a cell surface receptor for xenotropic and polytropic murine leukemia viruses. Proc. Nat. Aca.

Further studies establishing the function and utilities of XPR1 are found in John Hopkins OMIM database record ID 605237, and in sited publications numbered 1685-1688 listed in the bibliography section hereinbelow, which are also hereby incorporated by reference. FIG. 1 further provides a conceptual description of a novel bioinformatically detected viral gene of the present invention, referred to here as Viral Genomic Address Messenger 126 (VGAM126) viral gene, which modulates expression of respective host target genes thereof, the function and utility of which host target genes is known in the art.

VGAM126 is a novel bioinformatically detected regulatory, non protein coding, viral micro RNA (miRNA) gene. The method by which VGAM126 was detected is described hereinabove with reference to FIGS. 1-8.

VGAM126 gene, herein designated VGAM GENE, is a viral gene contained in the genome of Vaccinia Virus. VGAM126 host target gene, herein designated VGAM HOST TARGET GENE, is a human gene contained in the human genome.

VGAM126 gene encodes a VGAM126 precursor RNA, herein designated VGAM PRECURSOR RNA. Similar to other miRNA genes, and unlike most ordinary genes, VGAM126 precursor RNA does not encode a protein. A nucleotide sequence identical or highly similar to the nucleotide sequence of VGAM126 precursor RNA is designated SEQ ID:112, and is provided hereinbelow with reference to the sequence listing part. Nucleotide sequence SEQ ID:112 is located at position 53786 relative to the genome of Vaccinia Virus.

VGAM126 precursor RNA folds onto itself, forming VGAM126 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM126 folded precursor RNA into VGAM126 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM126 RNA is designated SEQ ID:461, and is provided hereinbelow with reference to the sequence listing part.

VGAM126 host target gene, herein designated VGAM HOST TARGET GENE, encodes a corresponding messenger RNA, VGAM126 host target RNA, herein designated VGAM HOST TARGET RNA. VGAM126 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM126 RNA, herein designated VGAM RNA, binds complementarily to one or more host target binding sites located in untranslated regions of VGAM126 host target RNA, herein designated VGAM HOST TARGET RNA. This complementary binding is due to the fact that the nucleotide sequence of VGAM126 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM126 RNA, herein designated VGAM RNA, may have a different number of host target binding sites in untranslated regions of a VGAM126 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM126 RNA, herein designated VGAM RNA, to host target binding sites on VGAM126 host target RNA, herein designated VGAM HOST TARGET RNA, such as BINDING SITE I, BINDING SITE II and BINDING SITE III, inhibits translation of VGAM126 host target RNA into VGAM126 host target protein, herein designated VGAM HOST TARGET PROTEIN. VGAM host target protein is therefore outlined by a broken line.

It is appreciated that VGAM126 host target gene, herein designated VGAM HOST TARGET GENE, in fact represents a plurality of VGAM126 host target genes. The mRNA of each one of this plurality of VGAM126 host target genes comprises one or more host target binding sites, each having a nucleotide sequence which is at least partly complementary to VGAM126 RNA, herein designated VGAM RNA, and which when bound by VGAM126 RNA causes inhibition of translation of respective one or more VGAM126 host target proteins.

It is further appreciated by one skilled in the art that the mode of translational inhibition illustrated by FIG. 1 with specific reference to translational inhibition exerted by VGAM126 gene, herein designated VGAM GENE, on one or more VGAM126 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM126 is inhibition of expression of host target genes, as part of a novel viral mechanism of attacking a host. Accordingly, utilities of VGAM126 include diagnosis, prevention and treatment of viral infection by Vaccinia Virus. Specific functions, and accordingly utilities, of VGAM126 correlate with, and may be deduced from, the identity of the host target genes which VGAM126 binds and inhibits, and the function of these host target genes, as elaborated hereinbelow.

Nucleotide sequences of the VGAM126 precursor RNA, herein designated VGAM PRECURSOR RNA, and of the ‘diced’ VGAM126 RNA, herein designated VGAM RNA, and a schematic representation of the secondary folding of VGAM126 folded precursor RNA, herein designated VGAM FOLDED PRECURSOR RNA, of VGAM126 are further described hereinbelow with reference to Table 1.

Nucleotide sequences of host target binding sites, such as BINDING SITE-I, BINDING SITE-II and BINDING SITE-III of FIG. 1, found on VGAM126 host target RNA, and schematic representation of the complementarity of each of these host target binding sites to VGAM126 RNA, herein designated VGAM RNA, are described hereinbelow with reference to Table 2.

As mentioned hereinabove with reference to FIG. 1, a function of VGAM126 gene, herein designated VGAM is inhibition of expression of VGAM126 target genes. It is appreciated that specific functions, and accordingly utilities, of VGAM126 correlate with, and may be deduced from, the identity of the target genes which VGAM126 binds and inhibits, and the function of these target genes, as elaborated hereinbelow.

Deafness, Autosomal Dominant 5 (DFNA5, Accession NM_{—}004403) is a VGAM126 host target gene. DFNA5 BINDING SITE is HOST TARGET binding site found in the 3′ untranslated region of mRNA encoded by DFNA5, corresponding to a HOST TARGET binding site such as BINDING SITE I, BINDING SITE II or BINDING SITE III. Table 2 illustrates the complementarity of the nucleotide sequences of DFNA5 BINDING SITE, designated SEQ ID:1109, to the nucleotide sequence of VGAM126 RNA, herein designated VGAM RNA, also designated SEQ ID:461.

A function of VGAM126 is therefore inhibition of Deafness, Autosomal Dominant 5 (DFNA5, Accession NM_{—}004403). Accordingly, utilities of VGAM126 include diagnosis, prevention and treatment of diseases and clinical conditions associated with DFNA5. Lecithin Retinol Acyltransferase (phosphatidyIcholine—retinol O-acyltransferase) (LRAT, Accession XM_{—}011181) is another VGAM126 host target gene. LRAT BINDING SITE is HOST TARGET binding site found in the 3′ untranslated region of mRNA encoded by LRAT, corresponding to a HOST TARGET binding site such as BINDING SITE I, BINDING SITE II or BINDING SITE III. Table 2 illustrates the complementarity of the nucleotide sequences of LRAT BINDING SITE, designated SEQ ID:2557, to the nucleotide sequence of VGAM126 RNA, herein designated VGAM RNA, also designated SEQ ID:461.

Another function of VGAM126 is therefore inhibition of Lecithin Retinol Acyltransferase (phosphatidyIcholine—retinol O-acyltransferase) (LRAT, Accession XM_{—}011181). Accordingly, utilities of VGAM126 include diagnosis, prevention and treatment of diseases and clinical conditions associated with LRAT. Polycystic Kidney and Hepatic Disease 1 (autosomal recessive) (PKHD1, Accession NM_{—}138694) is another VGAM126 host target gene. PKHD1 BINDING SITE is HOST TARGET binding site found in the 3′ untranslated region of mRNA encoded by PKHD1, corresponding to a HOST TARGET binding site such as BINDING SITE I, BINDING SITE II or BINDING SITE III. Table 2 illustrates the complementarity of the nucleotide sequences of PKHD1 BINDING SITE, designated SEQ ID:2449, to the nucleotide sequence of VGAM126 RNA, herein designated VGAM RNA, also designated SEQ ID:461.

Another function of VGAM126 is therefore inhibition of Polycystic Kidney and Hepatic Disease 1 (autosomal recessive) (PKHD1, Accession NM_{—}138694). Accordingly, utilities of VGAM126 include diagnosis, prevention and treatment of diseases and clinical conditions associated with PKHD1. HSPC055 (Accession NM_{—}014153) is another VGAM126 host target gene. HSPC055 BINDING SITE is HOST TARGET binding site found in the 5′ untranslated region of mRNA encoded by HSPC055, corresponding to a HOST TARGET binding site such as BINDING SITE I, BINDING SITE II or BINDING SITE III. Table 2 illustrates the complementarity of the nucleotide sequences of HSPC055 BINDING SITE, designated SEQ ID: 1480, to the nucleotide sequence of VGAM126 RNA, herein designated VGAM RNA, also designated SEQ ID:461.

Another function of VGAM126 is therefore inhibition of HSPC055 (Accession NM_{—}014153). Accordingly, utilities of VGAM126 include diagnosis, prevention and treatment of diseases and clinical conditions associated with HSPC055. PRO0386 (Accession NM_{—}018562) is another VGAM126 host target gene. PRO0386 BINDING SITE is HOST TARGET binding site found in the 5′ untranslated region of mRNA encoded by PRO0386, corresponding to a HOST TARGET binding site such as BINDING SITE I, BINDING SITE II or BINDING SITE III. Table 2 illustrates the complementarity of the nucleotide sequences of PRO0386 BINDING SITE, designated SEQ ID:1845, to the nucleotide sequence of VGAM126 RNA, herein designated VGAM RNA, also designated SEQ ID:461.

Another function of VGAM126 is therefore inhibition of PRO0386 (Accession NM_{—}018562). Accordingly, utilities of VGAM126 include diagnosis, prevention and treatment of diseases and clinical conditions associated with PRO0386. LOC121441 (Accession XM_{—}058561) is another VGAM126 host target gene. LOC121441 BINDING SITE is HOST TARGET binding site found in the 3′ untranslated region of mRNA encoded by LOC121441, corresponding to a HOST TARGET binding site such as BINDING SITE I, BINDING SITE II or BINDING SITE III. Table 2 illustrates the complementarity of the nucleotide sequences of LOC121441 BINDING SITE, designated SEQ ID:2992, to the nucleotide sequence of VGAM126 RNA, herein designated VGAM RNA, also designated SEQ ID:461.

Another function of VGAM126 is therefore inhibition of LOC121441 (Accession XM_{—}058561). Accordingly, utilities of VGAM126 include diagnosis, prevention and treatment of diseases and clinical conditions associated with LOC121441. FIG. 1 further provides a conceptual description of a novel bioinformatically detected viral gene of the present invention, referred to here as Viral Genomic Address Messenger 127 (VGAM127) viral gene, which modulates expression of respective host target genes thereof, the function and utility of which host target genes is known in the art.

VGAM127 is a novel bioinformatically detected regulatory, non protein coding, viral micro RNA (miRNA) gene. The method by which VGAM127 was detected is described hereinabove with reference to FIGS. 1-8.

VGAM127 gene, herein designated VGAM GENE, is a viral gene contained in the genome of Vaccinia Virus. VGAM127 host target gene, herein designated VGAM HOST TARGET GENE, is a human gene contained in the human genome.

VGAM127 gene encodes a VGAM127 precursor RNA, herein designated VGAM PRECURSOR RNA. Similar to other miRNA genes, and unlike most ordinary genes, VGAM127 precursor RNA does not encode a protein. A nucleotide sequence identical or highly similar to the nucleotide sequence of VGAM127 precursor RNA is designated SEQ ID:113, and is provided hereinbelow with reference to the sequence listing part. Nucleotide sequence SEQ ID:113 is located at position 56050 relative to the genome of Vaccinia Virus.

VGAM127 precursor RNA folds onto itself, forming VGAM127 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM127 folded precursor RNA into VGAM127 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM127 RNA is designated SEQ ID:462, and is provided hereinbelow with reference to the sequence listing part.

VGAM127 host target gene, herein designated VGAM HOST TARGET GENE, encodes a corresponding messenger RNA, VGAM127 host target RNA, herein designated VGAM HOST TARGET RNA. VGAM127 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM127 RNA, herein designated VGAM RNA, binds complementarily to one or more host target binding sites located in untranslated regions of VGAM127 host target RNA, herein designated VGAM HOST TARGET RNA. This complementary binding is due to the fact that the nucleotide sequence of VGAM127 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM127 RNA, herein designated VGAM RNA, may have a different number of host target binding sites in untranslated regions of a VGAM127 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM127 RNA, herein designated VGAM RNA, to host target binding sites on VGAM127 host target RNA, herein designated VGAM HOST TARGET RNA, such as BINDING SITE I, BINDING SITE II and BINDING SITE III, inhibits translation of VGAM127 host target RNA into VGAM127 host target protein, herein designated VGAM HOST TARGET PROTEIN. VGAM host target protein is therefore outlined by a broken line.

It is appreciated that VGAM127 host target gene, herein designated VGAM HOST TARGET GENE, in fact represents a plurality of VGAM127 host target genes. The mRNA of each one of this plurality of VGAM127 host target genes comprises one or more host target binding sites, each having a nucleotide sequence which is at least partly complementary to VGAM127 RNA, herein designated VGAM RNA, and which when bound by VGAM127 RNA causes inhibition of translation of respective one or more VGAM127 host target proteins.

It is further appreciated by one skilled in the art that the mode of translational inhibition illustrated by FIG. 1 with specific reference to translational inhibition exerted by VGAM127 gene, herein designated VGAM GENE, on one or more VGAM127 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM127 is inhibition of expression of host target genes, as part of a novel viral mechanism of attacking a host. Accordingly, utilities of VGAM127 include diagnosis, prevention and treatment of viral infection by Vaccinia Virus. Specific functions, and accordingly utilities, of VGAM127 correlate with, and may be deduced from, the identity of the host target genes which VGAM127 binds and inhibits, and the function of these host target genes, as elaborated hereinbelow.

Nucleotide sequences of the VGAM127 precursor RNA, herein designated VGAM PRECURSOR RNA, and of the ‘diced’ VGAM127 RNA, herein designated VGAM RNA, and a schematic representation of the secondary folding of VGAM127 folded precursor RNA, herein designated VGAM FOLDED PRECURSOR RNA, of VGAM127 are further described hereinbelow with reference to Table 1.

Nucleotide sequences of host target binding sites, such as BINDING SITE-I, BINDING SITE-II and BINDING SITE-III of FIG. 1, found on VGAM127 host target RNA, and schematic representation of the complementarity of each of these host target binding sites to VGAM127 RNA, herein designated VGAM RNA, are described hereinbelow with reference to Table 2.

As mentioned hereinabove with reference to FIG. 1, a function of VGAM127 gene, herein designated VGAM is inhibition of expression of VGAM127 target genes. It is appreciated that specific functions, and accordingly utilities, of VGAM127 correlate with, and may be deduced from, the identity of the target genes which VGAM127 binds and inhibits, and the function of these target genes, as elaborated hereinbelow.

A Disintegrin-like and Metalloprotease (reprolysin type) with Thrombospondin Type 1 Motif, 5 (aggrecanase-2) (ADAMTS5, Accession NM_{—}007038) is a VGAM127 host target gene. ADAMTS5 BINDING SITE is HOST TARGET binding site found in the 3′ untranslated region of mRNA encoded by ADAMTS5, corresponding to a HOST TARGET binding site such as BINDING SITE I, BINDING SITE II or BINDING SITE III. Table 2 illustrates the complementarity of the nucleotide sequences of ADAMTS5 BINDING SITE, designated SEQ ID:1351, to the nucleotide sequence of VGAM127 RNA, herein designated VGAM RNA, also designated SEQ ID:462.

A function of VGAM127 is therefore inhibition of A Disintegrin-like and Metalloprotease (reprolysin type) with Thrombospondin Type 1 Motif, 5 (aggrecanase-2) (ADAMTS5, Accession NM_{—}007038), a gene which cleaves aggrecan, a cartilage proteoglycan, and may be involved in its turnover. Accordingly, utilities of VGAM127 include diagnosis, prevention and treatment of diseases and clinical conditions associated with ADAMTS5. The function of ADAMTS5 and its association with various diseases and clinical conditions, has been established by previous studies, as described hereinabove with reference to VGAM49. Basic Helix-loop-helix Domain Containing, Class B, 3 (BHLHB3, Accession NM_{—}030762) is another VGAM127 host target gene. BHLHB3 BINDING SITE is HOST TARGET binding site found in the 3′ untranslated region of mRNA encoded by BHLHB3, corresponding to a HOST TARGET binding site such as BINDING SITE I, BINDING SITE II or BINDING SITE III. Table 2 illustrates the complementarity of the nucleotide sequences of BHLHB3 BINDING SITE, designated SEQ ID:2161, to the nucleotide sequence of VGAM127 RNA, herein designated VGAM RNA, also designated SEQ ID:462.

Another function of VGAM127 is therefore inhibition of Basic Helix-loop-helix Domain Containing, Class B, 3 (BHLHB3, Accession NM_{—}030762), a gene which represses both basal and activated transcription. Accordingly, utilities of VGAM127 include diagnosis, prevention and treatment of diseases and clinical conditions associated with BHLHB3. The function of BHLHB3 has been established by previous studies. By searching EST databases for sequences similar to DEC1, followed by 5-prime and 3-prime RACE with chondrocyte cDNA, Fujimoto et al. (2001) obtained cDNAs encoding human and mouse DEC2. The deduced 482-amino acid human DEC2 protein contains a bHLH domain and an Orange domain that are highly conserved with those of mouse Dec2 and rat Sharp1. DEC2 also has a C-terminal alanine/glycine-rich region not seen in DEC1. Northern blot analysis detected a 3.6-kb DEC2 transcript that was highly expressed in skeletal muscle and brain, moderately expressed in pancreas and heart, expressed at low levels in placenta and lung, and expressed at very low levels in liver and kidney. RT-PCR analysis detected ubiquitous but variable expression of DEC2. Using yeast 1-hybrid screens and reporter analysis, Garriga-Canut et al. (2001) showed that rat Sharp1 binds to the M1 muscarinic acetylcholine receptor (see OMIM Ref. No. CHRM1; 118510) and acts as a transcriptional repressor of both TATA-containing and TATA-less promoters. Repression occurs either via the bHLH domain or via a C-terminal domain that is sensitive to the histone deacetylase inhibitor trichostatin A.

Full details of the abovementioned studies are described in the following publications, the disclosure of which are hereby incorporated by reference:
Garriga-Canut, M.; Roopra, A.; Buckley, N. J.: The basic helix-loop-helix protein, SHARP-1, represses transcription by a histone deacetylase-dependent and histone deacetylase-independent mechanism. J. Biol. Chem. 276: 14821-14828, 2001.; and
Fujimoto, K.; Shen, M.; Noshiro, M.; Matsubara, K.; Shingu, S.; Honda, K.; Yoshida, E.; Suardita, K.; Matsuda, Y.; Kato, Y.: Molecular cloning and characterization of DEC2, a new memb.

Further studies establishing the function and utilities of BHLHB3 are found in John Hopkins OMIM database record ID 606200, and in sited publications numbered 210-211 listed in the bibliography section hereinbelow, which are also hereby incorporated by reference. Chemokine (C-C motif) Receptor 2 (CCR2, Accession NM_{—}000647) is another VGAM127 host target gene. CCR2 BINDING SITE is HOST TARGET binding site found in the 3′ untranslated region of mRNA encoded by CCR2, corresponding to a HOST TARGET binding site such as BINDING SITE I, BINDING SITE II or BINDING SITE III. Table 2 illustrates the complementarity of the nucleotide sequences of CCR2 BINDING SITE, designated SEQ ID:767, to the nucleotide sequence of VGAM127 RNA, herein designated VGAM RNA, also designated SEQ ID:462.

Another function of VGAM127 is therefore inhibition of Chemokine (C-C motif) Receptor 2 (CCR2, Accession NM_{—}000647), a gene which binds chemokines and transduces a signal by increasing the intracellular calcium ions level. Accordingly, utilities of VGAM127 include diagnosis, prevention and treatment of diseases and clinical conditions associated with CCR2. The function of CCR2 has been established by previous studies. Charo et al. (1994) isolated 2 cDNAs by degenerate PCR using primers for a conserved region in the second and third transmembrane domains of the MIP-1-alpha/RANTES receptor (OMIM Ref. No. 601159) and IL-8 receptors (146928, 146929). They then used PCR products to screen a human monocytic leukemia-cell library. The 2 cDNAs encoded putative chemokine receptors (termed A and B) that are identical except for their C termini and thus appear to result from alternative splicing. The 347-amino acid predicted protein from the A isoform, designated MCP1RA (and later termed CC CKR2A by Combadiere et al., 1995), is 51% identical to the MIP-1-alpha/RANTES receptor. As are the other members of this receptor family, MCP1RA is a 7-transmembrane G protein-coupled receptor. Combadiere et al. (1995) demonstrated that the predominant agonist for CC CKR2A is MCP1, while both MCP1 and MCP3 (OMIM Ref. No. 158106) are ligands for the CC CKR2β form (Combadiere et al., 1995). Animal model experiments lend further support to the function of CCR2. Peters et al. (2000) observed that after immunization with Th1-inducing agents, Ccr2 −/− mice produced markedly less gamma-interferon (IFNG; 147570) after antigen-specific stimulation than did wildtype mice. In contrast, IL5 (OMIM Ref. No. 147850), IL10 (OMIM Ref. No. 124092), and IL13 (OMIM Ref. No. 147683) production was not impaired in Ccr2 −/− mice. Flow cytometric analysis showed that fewer antigen-presenting cells migrated to the immunization site or draining lymph nodes in the Ccr2 −/− mice. Peters et al. (2000) concluded that CCR2 is required for proper trafficking of antigen-presenting cells capable of inducing IFNG production by T cells.

It is appreciated that the abovementioned animal model for CCR2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Combadiere, C.; Ahuja, S. K.; Van Damme, J.; Tiffany, H. L.; Gao, J.-L.; Murphy, P. M.: Monocyte chemoattractant protein-3 is a functional ligand for CC chemokine receptors 1 and 2B. J. Biol. Chem. 270: 29671-29675, 1995.; and
Peters, W.; Dupuis, M.; Charo, I. F.: A mechanism for the impaired IFN-gamma production in C-C chemokine receptor 2 (CCR2) knockout mice: role of CCR2 in linking the innate and adaptive.

Further studies establishing the function and utilities of CCR2 are found in John Hopkins OMIM database record ID 601267, and in sited publications numbered 2084-2085, 2090, 2095-209 and 2138-2105 listed in the bibliography section hereinbelow, which are also hereby incorporated by reference. Decorin (DCN, Accession NM_{—}001920) is another VGAM127 host target gene. DCN BINDING SITE1 through DCN BINDING SITE6 are HOST TARGET binding sites found in untranslated regions of mRNA encoded by DCN, corresponding to HOST TARGET binding sites such as BINDING SITE I, BINDING SITE II or BINDING SITE III. Table 2 illustrates the complementarity of the nucleotide sequences of DCN BINDING SITE1 through DCN BINDING SITE6, designated SEQ ID:868, SEQ ID:2421, SEQ ID:2422, SEQ ID:2423, SEQ ID:2424 and SEQ ID:2425 respectively, to the nucleotide sequence of VGAM127 RNA, herein designated VGAM RNA, also designated SEQ ID:462.

Another function of VGAM127 is therefore inhibition of Decorin (DCN, Accession NM_{—}001920), a gene which may mediate in epithelial/mesenchymal interactions. Accordingly, utilities of VGAM127 include diagnosis, prevention and treatment of diseases and clinical conditions associated with DCN. The function of DCN has been established by previous studies. Decorin and biglycan (OMIM Ref. No. 301870) are related but distinct small proteoglycans found in many connective tissues. Danielson et al. (1993) found that the human decorin gene spans more than 38 kb and contains 8 exons and very large introns, 2 of which are 5.4 and more than 13.2 kb. They discovered 2 alternatively spliced leader exons, 1a and 1b, in the 5-prime untranslated region. Using Northern blotting or reverse transcriptase PCR, they detected the 2 leader exons in a variety of mRNAs isolated from human cell lines and tissues. Sequences highly homologous (OMIM Ref. No. 74-87%) to exons 1a and 1b were found in the 5-prime untranslated region of avian and bovine decorin, respectively. This high degree of conservation among species suggested regulatory functions for these leader exons. In situ hybridization studies of developing mouse embryos suggested that decorin may play a role in epithelial/mesenchymal interactions during organ development and shaping (Scholzen et al., 1994). Dyne et al. (1996) studied 2 patients with osteogenesis imperfecta and the same gly415-to-ser mutation of the COL1A1 gene (120150.0044), but a different clinical expression. They speculated that these differences could be the result of abnormalities in other connective tissue proteins. Since decorin is a component of connective tissue, binds to type I collagen fibrils, and plays a role in matrix assembly, they studied decorin production in skin fibroblasts from these 2 patients. Cultured fibroblasts from the patient with extremely severe osteogenesis imperfecta (classified as type II/III) were found to secrete barely detectable amounts of decorin into culture medium. Northern blot analysis showed decorin mRNA levels below the limit of detection. The patient with a less severe phenotype had fibroblasts that expressed decorin normally. Dyne et al. (1996) suggested that the different clinical phenotypes could be due to the differing genetic backgrounds of the patients, such that in the more severely affected patient the absence of decorin aggravated the clinical phenotype.

Full details of the abovementioned studies are described in the following publications, the disclosure of which are hereby incorporated by reference:
Scholzen, T.; Solursh, M.; Suzuki, S.; Reiter, R.; Morgan, J. L.; Buchberg, A. M.; Siracusa, L. D.; Iozzo, R. V.: The murine decorin: complete cDNA cloning, genomic organization, chromosomal assignment, and expression during organogenesis and tissue differentiation. J. Biol. Chem. 269: 28270-28281, 1994.; and
Dyne, K. M.; Valli, M.; Forlino, A.; Mottes, M.; Kresse, H.; Cetta, G.: Deficient expression of the small proteoglycan decorin in a case of severe/lethal osteogenesis imperfecta. Am. J.

Further studies establishing the function and utilities of DCN are found in John Hopkins OMIM database record ID 125255, and in sited publications numbered 446-455 listed in the bibliography section hereinbelow, which are also hereby incorporated by reference. Fatty Acid Synthase (FASN, Accession NM_{—}004104) is another VGAM127 host target gene. FASN BINDING SITE is HOST TARGET binding site found in the 3′ untranslated region of mRNA encoded by FASN, corresponding to a HOST TARGET binding site such as BINDING SITE I, BINDING SITE II or BINDING SITE III. Table 2 illustrates the complementarity of the nucleotide sequences of FASN BINDING SITE, designated SEQ ID:1088, to the nucleotide sequence of VGAM127 RNA, herein designated VGAM RNA, also designated SEQ ID:462.

Another function of VGAM127 is therefore inhibition of Fatty Acid Synthase (FASN, Accession NM_{—}004104), a gene which catalyzes the formation of long-chain fatty acids from acetyl-coa, malonyl-coa and nadph. Accordingly, utilities of VGAM127 include diagnosis, prevention and treatment of diseases and clinical conditions associated with FASN. The function of FASN has been established by previous studies. Fatty acid synthase (EC 2.3.1.85) catalyzes the conversion of acetyl-CoA and malonyl-CoA, in the presence of NADPH, into long-chain saturated fatty acids (Wakil, 1989). In prokaryotes and plants, FASN consists of an acyl carrier protein and 7 structurally independent monofunctional enzymes. In animals, however, all of the component enzymatic activities of FASN and acyl carrier protein are organized in one large polypeptide chain. Loftus et al. (2000) identified a link between anabolic energy metabolism and appetite control. Both systemic and intracerebroventricular treatment of mice with fatty acid synthase inhibitors (cerulenin and C75, a synthetic compound) led to inhibition of feeding and dramatic weight loss. C75 inhibited expression of the prophagic signal neuropeptide Y (OMIM Ref. No. 162640) in the hypothalamus and acted in a leptin (OMIM Ref. No. 164160)-independent manner that appears to be mediated by malonyl-CoA. Loftus et al. (2000) suggested that FASN may represent an important link in feeding regulation and may be a potential therapeutic target for obesity.

Full details of the abovementioned studies are described in the following publications, the disclosure of which are hereby incorporated by reference:
Loftus, T. M.; Jaworsky, D. E.; Frehywot, G. L.; Townsend, C. A.; Ronnett, G. V.; Lane, M. D.; Kuhajda, F. P.: Reduced food intake and body weight in mice treated with fatty acid synthase inhibitors. Science 288: 2379-2381, 2000.; and
Wakil, S. J.: Fatty acid synthase, a proficient multifunctional enzyme. Biochemistry 28: 4523-4530, 1989.

Further studies establishing the function and utilities of FASN are found in John Hopkins OMIM database record ID 600212, and in sited publications numbered 2441-2445 listed in the bibliography section hereinbelow, which are also hereby incorporated by reference. Inhibin, Beta B (activin AB beta polypeptide) (INHBB, Accession NM_{—}002193) is another VGAM127 host target gene. INHBB BINDING SITE is HOST TARGET binding site found in the 3′ untranslated region of mRNA encoded by INHBB, corresponding to a HOST TARGET binding site such as BINDING SITE I, BINDING SITE II or BINDING SITE III. Table 2 illustrates the complementarity of the nucleotide sequences of INHBB BINDING SITE, designated SEQ ID:905, to the nucleotide sequence of VGAM127 RNA, herein designated VGAM RNA, also designated SEQ ID:462.

Another function of VGAM127 is therefore inhibition of Inhibin, Beta B (activin AB beta polypeptide) (INHBB, Accession NM_{—}002193), a gene which inhibins inhibit the secretion of follitropin by the pituitary gland. Accordingly, utilities of VGAM127 include diagnosis, prevention and treatment of diseases and clinical conditions associated with INHBB. The function of INHBB has been established by previous studies. The activins, dimers of beta-A or beta-B subunits encoded by the genes Inhba (OMIM Ref. No. 147290) and Inhbb, respectively, are TGF-beta (see OMIM Ref. No. 190180) super family members that have roles in reproduction and development (Brown et al., 2000). Activin ligands act as growth and differentiation factors in many cells and tissues. Mellor et al. (2000) examined the localization of and dimerization among activin subunits. The results demonstrated that activin beta-C (see OMIM Ref. No. 601233) can form dimers with activin beta-A and beta-B in vitro, but not with the inhibin alpha subunit (OMIM Ref. No. 147380). Using a specific antibody, activin beta-C protein was localized to human liver and prostate and colocalized with beta-A and beta-B subunits to specific cell types in benign and malignant prostate tissues. The capacity to form novel activin heterodimers (but not inhibin C) appears to reside in the human liver and prostate. The authors concluded that formation of activin AC or BC heterodimers may have significant implications in the regulation of levels and/or biologic activity of other activins in these tissues. Malignancy of pheochromocytomas is difficult to estimate on the basis of histopathologic features. In a search for new markers to differentiate malignant pheochromocytomas from benign ones, Salmenkivi et al. (2001) tested the value of inhibin/activin subunit expression. Inhibins are heterodimeric glycoproteins consisting of an alpha subunit and either a beta-A or a beta-B subunit. Activins are composed of beta subunits only. Immunohistochemically, inhibin/activin beta-B subunit was strongly positive in the normal adrenal medulla, but the cortex was negative. A striking difference was found in inhibin/activin beta-B expression between benign and malignant pheochromocytomas. The majority of benign adrenal tumors (27 of 30) showed strong or moderate immunoreactivity, whereas all 7 malignant tumors were negative or only weakly positive for inhibin/activin beta-B subunit. Salmenkivi et al. (2001) suggested that inhibin/activin beta-B subunit is expressed in normal adrenal medullary cells. Strong staining was found in most benign adrenal pheochromocytomas, whereas malignant tumors were almost negative. They concluded that loss of inhibin/activin beta-B subunit expression in pheochromocytomas may be used as an indicator of malignant potential. Animal model experiments lend further support to the function of INHBB. Whereas mice homozygous for the Inhba-null allele demonstrate disruption of whisker, palate, and tooth development leading to neonatal lethality, homozygous Inhbb-null mice are viable, fertile, and have eye defects. To determine if these phenotypes were due to spatiotemporal expression differences of the ligands or disruption of specific ligand-receptor interactions, Brown et al. (2000) replaced the region of Inhba encoding the mature protein with Inhbb, creating the allele designated Inhba(BK). Although the craniofacial phenotypes of the Inhba-null mutation were rescued by the Inhba(BK) allele, somatic, testicular, genital, and hair growth were grossly affected and influenced by the dosage and bioactivity of the allele. Thus, Brown et al. (2000) concluded that functional compensation within the TGF-beta super family can occur if the replacement gene is expressed appropriately. The novel phenotypes in these mice further illustrate the usefulness of insertion strategies for defining protein function. The structural organization of the testes of adult Inhba(BK/BK) mice was normal; however, the differentiation of the seminiferous tubules of Inhba(BK/−) mice was delayed. The testicular volumes of both Inhba(BK/BK) and Inhba(BK/−) mice were less than those of controls, and the dosage of the Inhba(BK) allele correlated positively with testicular size. Inhba(+/BK) males had normal onset of fertility, whereas Inhba(BK/BK) males had delayed onset of fertility similar to Acvr2 (OMIM Ref. No. 102581) −/− mice. Only 1 in 6 Inhba(BK/BK) females produced litters, whereas Inhba(+/BK) females were normally fertile. The ovaries of Inhba(BK/−) mice were smaller and contained fewer large preantral follicles than those of controls. Inhba(BK/BK) and Inhba(BK/−) mice were identified by their smaller size, slower hair growth, the rough appearance of their fur, and sunken eyes. Approximately 50% of Inhba(BK/BK) mice died by 26 weeks, whereas Inhba(BK/−) mice invariably became cachectic and died between 3 and 4 weeks. The summary of phenotypic findings of Inhba(BK/−) mice includes short whiskers, normal tooth development, no cleft palate, symmetric growth deficiency (OMIM Ref. No. severe), enlargement of external genitalia, hypogonadism (OMIM Ref. No. severe), delayed hair growth (moderate), hypoglycemia (mild), decreased life expectancy (OMIM Ref. No. severe), and anemia

It is appreciated that the abovementioned animal model for INHBB is acknowledged by those skilled in the art as a scientifically valid animal model, as can be further appreciated from the publications sited hereinbelow.

Full details of the abovementioned studies are described in the following publications, the disclosure of which are hereby incorporated by reference:
Salmenkivi, K.; Arola, J.; Voutilainen, R.; Ilvesmaki, V.; Haglund, C.; Kahri, A. I.; Heikkila, P.; Liu, J.: Inhibin/activin beta-B-subunit expression in pheochromocytomas favors benign diagnosis. J. Clin. Endocr. Metab. 86: 2231-2235, 2001.; and
Brown, C. W.; Houston-Hawkins, D. E.; Woodruff, T. K.; Matzuk, M. M.: Insertion of Inhbb into the Inhba locus rescues the Inhba-null phenotype and reveals new activin functions. Nature.

Further studies establishing the function and utilities of INHBB are found in John Hopkins OMIM database record ID 147390, and in sited publications numbered 1190, 2624-119 and 2625-2626 listed in the bibliography section hereinbelow, which are also hereby incorporated by reference. Neural Precursor Cell Expressed, Developmentally Down-regulated 4 (NEDD4, Accession XM_{—}046129) is another VGAM127 host target gene. NEDD4 BINDING SITE is HOST TARGET binding site found in the 3′ untranslated region of mRNA encoded by NEDD4, corresponding to a HOST TARGET binding site such as BINDING SITE I, BINDING SITE II or BINDING SITE III. Table 2 illustrates the complementarity of the nucleotide sequences of NEDD4 BINDING SITE, designated SEQ ID:2875, to the nucleotide sequence of VGAM127 RNA, herein designated VGAM RNA, also designated SEQ ID:462.

Another function of VGAM127 is therefore inhibition of Neural Precursor Cell Expressed, Developmentally Downregulated 4 (NEDD4, Accession XM_{—}046129), a gene which ubiquitinates regulatory proteins involved in transcription. Accordingly, utilities of VGAM127 include diagnosis, prevention and treatment of diseases and clinical conditions associated with NEDD4. The function of NEDD4 has been established by previous studies. Kumar et al. (1992) identified Nedd4 as one of a group of mouse genes that show developmentally regulated expression in mouse embryonic brain. Kumar et al. (1997) showed that Nedd4 is expressed in various other embryonic tissues and persists in most adult tissues. Using antibody raised against a fusion protein, they demonstrated that the Nedd4 protein is localized to the cellular cytoplasm. Kumar et al. (1997) reported that the human NEDD4 protein has 86% amino acid identity with the mouse protein. It has homology to ubiquitin-protein ligases and contains 4 protein-protein interaction (WW) domains and a calcium/phospholipid binding domain. Imhof and McDonnell (1996) found that both human NEDD4 and yeast RSP5 potentiate hormone-dependent activation of transcription by the human progesterone and glucocorticoid receptors. They used mutant proteins to show that neither the ubiquitin-protein ligase activity nor the WW domains are absolutely required for the potentiation of the steroid receptors.

Full details of the abovementioned studies are described in the following publications, the disclosure of which are hereby incorporated by reference:
Imhof, M. O.; McDonnell, D. P.: Yeast RSP5 and its human homolog hRPF1 potentiate hormone-dependent activation of transcription by human progesterone and glucocorticoid receptors. Molec. Cell. Biol. 16: 2594-2605, 1996.; and
Kumar, S.; Harvey, K. F.; Kinoshita, M.; Copeland, N. G.; Noda, M.; Jenkins, N. A.: cDNA cloning, expression analysis, and mapping of the mouse Nedd4 gene. Genomics 40: 435-443, 1997.

Further studies establishing the function and utilities of NEDD4 are found in John Hopkins OMIM database record ID 602278, and in sited publications numbered 1712-64 and 336 listed in the bibliography section hereinbelow, which are also hereby incorporated by reference. Nuclear Transcription Factor Y, Alpha (NFYA, Accession NM_{—}021705) is another VGAM127 host target gene. NFYA BINDING SITE1 and NFYA BINDING SITE2 are HOST TARGET binding sites found in untranslated regions of mRNA encoded by NFYA, corresponding to HOST TARGET binding sites such as BINDING SITE I, BINDING SITE II or BINDING SITE III. Table 2 illustrates the complementarity of the nucleotide sequences of NFYA BINDING SITE1 and NFYA BINDING SITE2, designated SEQ ID:1955 and SEQ ID:933 respectively, to the nucleotide sequence of VGAM127 RNA, herein designated VGAM RNA, also designated SEQ ID:462.

Another function of VGAM127 is therefore inhibition of Nuclear Transcription Factor Y, Alpha (NFYA, Accession NM_{—}021705), a gene which stimulates the transcription of various genes by recognizing and binding to a ccaat motif in promoters. Accordingly, utilities of VGAM127 include diagnosis, prevention and treatment of diseases and clinical conditions associated with NFYA. The function of NFYA has been established by previous studies. NF-Y is a transcription factor thought to be essential for expression of the class II genes of the major histocompatibility complex (MHC; OMIM Ref. No. 142800). It recognizes a CCAAT motif upstream of gene promoters and is probably involved in the regulation of a variety of genes, including those for albumin (OMIM Ref. No. 103600), alpha-globin (OMIM Ref. No. 141800), collagen (see OMIM Ref. No. 120150), and beta-actin (OMIM Ref. No. 102630). NF-Y is composed of 2 subunits, NFYA and NFYB (OMIM Ref. No. 189904), both of which are necessary for DNA binding. This 2-subunit DNA-binding unit has been well conserved during evolution. NFYA and NFYB show striking sequence similarity with the yeast transcription factors Hap2 and Hap3, both of which are required for specific binding to a CCAAT-like motif. By in situ hybridization and analysis of somatic cell hybrids, L1 et al. (1991) assigned the NFYA gene to 6p21 (close to MHC) and the NFYB gene to human chromosome 12. After in situ hybridization, the maximum concentration of grains was in the region 12q22-q23. By Southern blot analysis of recombinant inbred lines and by in situ hybridization, the Nfya and Nfyb genes were assigned to mouse chromosome 17 and mouse chromosome 10, respectively. CCAAT, an upstream sequence element found in a multitude of higher eukaryotic promoters, serves as the recognition sequence for a variety of mammalian transcription factors. There are at least 3 chromatographically separable CCAAT-binding factors in HeLa cells: CP1, CP2, and CTF/NF-1 (OMIM Ref. No. 600729). These factors recognize overlapping but distinct subsets of known CCAAT-containing promoters and make distinguishable patterns of contacts with DNA in and around the CCAAT motif. Becker et al. (1991) noted that of these 3 factors, CP1 bears the greatest resemblance to the yeast Hap complex. This complex of 3 genes, Hap2, Hap3, and Hap4, is required for the expression of respiration in Saccharomyces cerevisiae—in particular, for expression of the principal isoform of cytochrome C (CYC1; 123980). In the yeast, 3 proteins are associated in a heteromeric complex that binds to an upstream activation sequence in the CYC1 promoter. Like Hap2/3/4, CYP in the human consists of a heteromeric association of at least 2 components, CP1A and CP1B, both of which are required for binding. Most strikingly, the subunits of CP1 and Hap2/3/4 can be interchanged in vitro. Thus, CP1 likely represents the human homolog of the yeast Hap complex, with the CP1B fraction containing the Hap2 homolog and the CP1A fraction containing a Hap3 equivalent. Becker et al. (1991) reported the isolation of a HeLa cDNA whose expression in S. cerevisiae corrected the respiratory defect in a strain bearing a Hap2 deletion. The cDNA encoding the human Hap2 homolog encodes a protein of 257 amino acids which has a 62-amino acid C-terminal region that shares 73% identity with the essential core region of Hap2.

Full details of the abovementioned studies are described in the following publications, the disclosure of which are hereby incorporated by reference:
Becker, D. M.; Fikes, J. D.; Guarente, L.: A cDNA encoding a human CCAAT-binding protein cloned by functional complementation in yeast. Proc. Nat. Acad. Sci. 88: 1968-1972, 1991.; and
Li, X.-Y.; Mattei, M. G.; Zaleska-Rutczynska, Z.; Hooft van Huijsduijnen, R.; Figueroa, F.; Nadeau, J.; Benoist, C.; Mathis, D.: One subunit of the transcription factor NF-Y maps close.

Further studies establishing the function and utilities of NFYA are found in John Hopkins OMIM database record ID 189903, and in sited publications numbered 40 and 3035 listed in the bibliography section hereinbelow, which are also hereby incorporated by reference. RAD17 Homolog (S. pombe) (RAD17, Accession NM_{—}133338) is another VGAM127 host target gene. RAD17 BINDING SITE1 through RAD17 BINDING SITE6 are HOST TARGET binding sites found in untranslated regions of mRNA encoded by RAD17, corresponding to HOST TARGET binding sites such as BINDING SITE I, BINDING SITE II or BINDING SITE III. Table 2 illustrates the complementarity of the nucleotide sequences of RAD17 BINDING SITE1 through RAD17 BINDING SITE6, designated SEQ ID:2410, SEQ ID:2411, SEQ ID:2412, SEQ ID:2413, SEQ ID:2414 and SEQ ID:1358 respectively, to the nucleotide sequence of VGAM127 RNA, herein designated VGAM RNA, also designated SEQ ID:462.

Another function of VGAM127 is therefore inhibition of RAD17 Homolog (S. pombe) (RAD17, Accession NM_{—}133338), a gene which may have a role in DNA damage-dependent and DNA replication-dependent cell cycle checkpoints. Accordingly, utilities of VGAM127 include diagnosis, prevention and treatment of diseases and clinical conditions associated with RAD17. The function of RAD17 has been established by previous studies. Cell cycle checkpoints are complex signal transduction pathways that ensure the coordination of the timing and order of cell cycle events. These checkpoint pathways play critical roles in maintaining genomic stability and integrity to prevent the development of cancer and hereditary diseases. In the fission yeast Schizosaccharomyces pombe, the rad17 gene is required for both the DNA damage-dependent and the DNA replication-dependent cell cycle checkpoints. Parker et al. (1998) identified expressed sequence tags corresponding to a human homolog of S. pombe rad17. By PCR, they isolated a human SK-N-MC neuroblastoma cell cDNA containing the complete open reading frame of this homolog, RAD17. The deduced 670-amino acid RAD17 protein has a calculated molecular mass of 71 kD and has 20% sequence identity to S. pombe rad17. Northern blot analysis detected an approximately 3.0-kb transcript in all tissues examined, with elevated levels in testis and cancer cell lines. Although human RAD17 did not complement the checkpoint phenotypes of an S. pombe rad17 mutant, it interacted with human RAD1 (OMIM Ref. No. 603153) in a yeast 2-hybrid system, and Parker et al. (1998) suggested that it is the homolog of S. pombe rad17.

Full details of the abovementioned studies are described in the following publications, the disclosure of which are hereby incorporated by reference:
Parker, A. E.; Van de Weyer, I.; Laus, M. C.; Verhasselt, P.; Luyten, W. H. M. L.: Identification of a human homologue of the Schizosaccharomyces pombe rad17+ checkpoint gene. J. Biol. Chem. 273: 18340-18346, 1998. Note: Erratum: J. Biol. Chem. 274: 24438-24439, 1999.; and
Bao, S.; Tibbetts, R. S.; Brumbaugh, K. M.; Fang, Y.; Richardson, D. A.; Ali, A.; Chen, S. M.; Abraham, R. T.; Wang, X.-F.: ATR/ATM-mediated phosphorylation of human Rad17 is required f.

Further studies establishing the function and utilities of RAD17 are found in John Hopkins OMIM database record ID 603139, and in sited publications numbered 1153, 228 and 1230-1231 listed in the bibliography section hereinbelow, which are also hereby incorporated by reference. DKFZp762E1511 (Accession XM_{—}003460) is another VGAM127 host target gene. DKFZp762E1511 BINDING SITE is HOST TARGET binding site found in the 3′ untranslated region of mRNA encoded by DKFZp762E1511, corresponding to a HOST TARGET binding site such as BINDING SITE I, BINDING SITE II or BINDING SITE III. Table 2 illustrates the complementarity of the nucleotide sequences of DKFZp762E1511 BINDING SITE, designated SEQ ID:2531, to the nucleotide sequence of VGAM127 RNA, herein designated VGAM RNA, also designated SEQ ID:462.

Another function of VGAM127 is therefore inhibition of DKFZp762E1511 (Accession XM_{—}003460). Accordingly, utilities of VGAM127 include diagnosis, prevention and treatment of diseases and clinical conditions associated with DKFZp762E1511. Eukaryotic Translation Initiation Factor 5 (EIF5, Accession NM_{—}001969) is another VGAM127 host target gene. EIF5 BINDING SITE is HOST TARGET binding site found in the 5′ untranslated region of mRNA encoded by EIF5, corresponding to a HOST TARGET binding site such as BINDING SITE I, BINDING SITE II or BINDING SITE III. Table 2 illustrates the complementarity of the nucleotide sequences of EIF5 BINDING SITE, designated SEQ ID:878, to the nucleotide sequence of VGAM127 RNA, herein designated VGAM RNA, also designated SEQ ID:462.

Another function of VGAM127 is therefore inhibition of Eukaryotic Translation Initiation Factor 5 (EIF5, Accession NM_{—}001969). Accordingly, utilities of VGAM127 include diagnosis, prevention and treatment of diseases and clinical conditions associated with EIF5. LATS, Large Tumor Suppressor, Homolog 1 (Drosophila) (LATS1, Accession XM_{—}015547) is another VGAM127 host target gene. LATS1 BINDING SITE is HOST TARGET binding site found in the 3′ untranslated region of mRNA encoded by LATS1, corresponding to a HOST TARGET binding site such as BINDING SITE I, BINDING SITE II or BINDING SITE III. Table 2 illustrates the complementarity of the nucleotide sequences of LATS1 BINDING SITE, designated SEQ ID:2563, to the nucleotide sequence of VGAM127 RNA, herein designated VGAM RNA, also designated SEQ ID:462.

Another function of VGAM127 is therefore inhibition of LATS, Large Tumor Suppressor, Homolog 1 (Drosophila) (LATS1, Accession XM_{—}015547). Accordingly, utilities of VGAM127 include diagnosis, prevention and treatment of diseases and clinical conditions associated with LATS1. MGC27277 (Accession NM_{—}144989) is another VGAM127 host target gene. MGC27277 BINDING SITE is HOST TARGET binding site found in the 3′ untranslated region of mRNA encoded by MGC27277, corresponding to a HOST TARGET binding site such as BINDING SITE I, BINDING SITE II or BINDING SITE III. Table 2 illustrates the complementarity of the nucleotide sequences of MGC27277 BINDING SITE, designated SEQ ID:2505, to the nucleotide sequence of VGAM127 RNA, herein designated VGAM RNA, also designated SEQ ID:462.

Another function of VGAM127 is therefore inhibition of MGC27277 (Accession NM_{—}144989). Accordingly, utilities of VGAM127 include diagnosis, prevention and treatment of diseases and clinical conditions associated with MGC27277. POLD3 (Accession XM_{—}166243) is another VGAM127 host target gene. POLD3 BINDING SITE is HOST TARGET binding site found in the 3′ untranslated region of mRNA encoded by POLD3, corresponding to a HOST TARGET binding site such as BINDING SITE I, BINDING SITE II or BINDING SITE III. Table 2 illustrates the complementarity of the nucleotide sequences of POLD3 BINDING SITE, designated SEQ ID:3538, to the nucleotide sequence of VGAM127 RNA, herein designated VGAM RNA, also designated SEQ ID:462.

Another function of VGAM127 is therefore inhibition of POLD3 (Accession XM_{—}166243). Accordingly, utilities of VGAM127 include diagnosis, prevention and treatment of diseases and clinical conditions associated with POLD3. TSLRP (Accession NM_{—}012472) is another VGAM127 host target gene. TSLRP BINDING SITE is HOST TARGET binding site found in the 5′ untranslated region of mRNA encoded by TSLRP, corresponding to a HOST TARGET binding site such as BINDING SITE I, BINDING SITE II or BINDING SITE III. Table 2 illustrates the complementarity of the nucleotide sequences of TSLRP BINDING SITE, designated SEQ ID:1435, to the nucleotide sequence of VGAM127 RNA, herein designated VGAM RNA, also designated SEQ ID:462.

Another function of VGAM127 is therefore inhibition of TSLRP (Accession NM_{—}012472). Accordingly, utilities of VGAM127 include diagnosis, prevention and treatment of diseases and clinical conditions associated with TSLRP. LOC147463 (Accession XM_{—}085799) is another VGAM127 host target gene. LOC147463 BINDING SITE is HOST TARGET binding site found in the 3′ untranslated region of mRNA encoded by LOC147463, corresponding to a HOST TARGET binding site such as BINDING SITE I, BINDING SITE II or BINDING SITE III. Table 2 illustrates the complementarity of the nucleotide sequences of LOC147463 BINDING SITE, designated SEQ ID:3108, to the nucleotide sequence of VGAM127 RNA, herein designated VGAM RNA, also designated SEQ ID:462.

Another function of VGAM127 is therefore inhibition of LOC147463 (Accession XM_{—}085799). Accordingly, utilities of VGAM127 include diagnosis, prevention and treatment of diseases and clinical conditions associated with LOC147463. LOC161734 (Accession XM_{—}102109) is another VGAM127 host target gene. LOC161734 BINDING SITE is HOST TARGET binding site found in the 3′ untranslated region of mRNA encoded by LOC161734, corresponding to a HOST TARGET binding site such as BINDING SITE I, BINDING SITE II or BINDING SITE III. Table 2 illustrates the complementarity of the nucleotide sequences of LOC161734 BINDING SITE, designated SEQ ID:3400, to the nucleotide sequence of VGAM127 RNA, herein designated VGAM RNA, also designated SEQ ID:462.

Another function of VGAM127 is therefore inhibition of LOC161734 (Accession XM_{—}102109). Accordingly, utilities of VGAM127 include diagnosis, prevention and treatment of diseases and clinical conditions associated with LOC161734. LOC164173 (Accession XM_{—}089424) is another VGAM127 host target gene. LOC164173 BINDING SITE is HOST TARGET binding site found in the 3′ untranslated region of mRNA encoded by LOC164173, corresponding to a HOST TARGET binding site such as BINDING SITE I, BINDING SITE II or BINDING SITE III. Table 2 illustrates the complementarity of the nucleotide sequences of LOC164173 BINDING SITE, designated SEQ ID:3235, to the nucleotide sequence of VGAM127 RNA, herein designated VGAM RNA, also designated SEQ ID:462.

Another function of VGAM127 is therefore inhibition of LOC164173 (Accession XM_{—}089424). Accordingly, utilities of VGAM127 include diagnosis, prevention and treatment of diseases and clinical conditions associated with LOC164173. LOC199675 (Accession XM_{—}113982) is another VGAM127 host target gene. LOC199675 BINDING SITE is HOST TARGET binding site found in the 3′ untranslated region of mRNA encoded by LOC199675, corresponding to a HOST TARGET binding site such as BINDING SITE I, BINDING SITE II or BINDING SITE III. Table 2 illustrates the complementarity of the nucleotide sequences of LOC199675 BINDING SITE, designated SEQ ID:3429, to the nucleotide sequence of VGAM127 RNA, herein designated VGAM RNA, also designated SEQ ID:462.

Another function of VGAM127 is therefore inhibition of LOC199675 (Accession XM_{—}113982). Accordingly, utilities of VGAM127 include diagnosis, prevention and treatment of diseases and clinical conditions associated with LOC199675. LOC200150 (Accession XM_{—}114131) is another VGAM127 host target gene. LOC200150 BINDING SITE is HOST TARGET binding site found in the 5′ untranslated region of mRNA encoded by LOC200150, corresponding to a HOST TARGET binding site such as BINDING SITE I, BINDING SITE II or BINDING SITE III. Table 2 illustrates the complementarity of the nucleotide sequences of LOC200150 BINDING SITE, designated SEQ ID:3439, to the nucleotide sequence of VGAM127 RNA, herein designated VGAM RNA, also designated SEQ ID:462.

Another function of VGAM127 is therefore inhibition of LOC200150 (Accession XM_{—}114131). Accordingly, utilities of VGAM127 include diagnosis, prevention and treatment of diseases and clinical conditions associated with LOC200150. LOC51185 (Accession NM_{—}016302) is another VGAM127 host target gene. LOC51185 BINDING SITE is HOST TARGET binding site found in the 3′ untranslated region of mRNA encoded by LOC51185, corresponding to a HOST TARGET binding site such as BINDING SITE I, BINDING SITE II or BINDING SITE III. Table 2 illustrates the complementarity of the nucleotide sequences of LOC51185 BINDING SITE, designated SEQ ID:1679, to the nucleotide sequence of VGAM127 RNA, herein designated VGAM RNA, also designated SEQ ID:462.

Another function of VGAM127 is therefore inhibition of LOC51185 (Accession NM_{—}016302). Accordingly, utilities of VGAM127 include diagnosis, prevention and treatment of diseases and clinical conditions associated with LOC51185. LOC90459 (Accession XM_{—}031826) is another VGAM127 host target gene. LOC90459 BINDING SITE is HOST TARGET binding site found in the 3′ untranslated region of mRNA encoded by LOC90459, corresponding to a HOST TARGET binding site such as BINDING SITE I, BINDING SITE II or BINDING SITE III. Table 2 illustrates the complementarity of the nucleotide sequences of LOC90459 BINDING SITE, designated SEQ ID:2643, to the nucleotide sequence of VGAM127 RNA, herein designated VGAM RNA, also designated SEQ ID:462.

Another function of VGAM127 is therefore inhibition of LOC90459 (Accession XM_{—}031826). Accordingly, utilities of VGAM127 include diagnosis, prevention and treatment of diseases and clinical conditions associated with LOC90459. LOC91694 (Accession XM_{—}040082) is another VGAM127 host target gene. LOC91694 BINDING SITE is HOST TARGET binding site found in the 5′ untranslated region of mRNA encoded by LOC91694, corresponding to a HOST TARGET binding site such as BINDING SITE I, BINDING SITE II or BINDING SITE III. Table 2 illustrates the complementarity of the nucleotide sequences of LOC91694 BINDING SITE, designated SEQ ID:2769, to the nucleotide sequence of VGAM127 RNA, herein designated VGAM RNA, also designated SEQ ID:462.

Another function of VGAM127 is therefore inhibition of LOC91694 (Accession XM_{—}040082). Accordingly, utilities of VGAM127 include diagnosis, prevention and treatment of diseases and clinical conditions associated with LOC91694. FIG. 1 further provides a conceptual description of a novel bioinformatically detected viral gene of the present invention, referred to here as Viral Genomic Address Messenger 128 (VGAM128) viral gene, which modulates expression of respective host target genes thereof, the function and utility of which host target genes is known in the art.

VGAM128 is a novel bioinformatically detected regulatory, non protein coding, viral micro RNA (miRNA) gene. The method by which VGAM128 was detected is described hereinabove with reference to FIGS. 1-8.

VGAM128 gene, herein designated VGAM GENE, is a viral gene contained in the genome of Vaccinia Virus. VGAM128 host target gene, herein designated VGAM HOST TARGET GENE, is a human gene contained in the human genome.

VGAM128 gene encodes a VGAM128 precursor RNA, herein designated VGAM PRECURSOR RNA. Similar to other miRNA genes, and unlike most ordinary genes, VGAM128 precursor RNA does not encode a protein. A nucleotide sequence identical or highly similar to the nucleotide sequence of VGAM128 precursor RNA is designated SEQ ID:114, and is provided hereinbelow with reference to the sequence listing part. Nucleotide sequence SEQ ID:114 is located at position 55816 relative to the genome of Vaccinia Virus.

VGAM128 precursor RNA folds onto itself, forming VGAM128 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM128 folded precursor RNA into VGAM128 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 93%) nucleotide sequence of VGAM128 RNA is designated SEQ ID:463, and is provided hereinbelow with reference to the sequence listing part.

VGAM128 host target gene, herein designated VGAM HOST TARGET GENE, encodes a corresponding messenger RNA, VGAM128 host target RNA, herein designated VGAM HOST TARGET RNA. VGAM128 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM128 RNA, herein designated VGAM RNA, binds complementarily to one or more host target binding sites located in untranslated regions of VGAM128 host target RNA, herein designated VGAM HOST TARGET RNA. This complementary binding is due to the fact that the nucleotide sequence of VGAM128 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM128 RNA, herein designated VGAM RNA, may have a different number of host target binding sites in untranslated regions of a VGAM128 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM128 RNA, herein designated VGAM RNA, to host target binding sites on VGAM128 host target RNA, herein designated VGAM HOST TARGET RNA, such as BINDING SITE I, BINDING SITE II and BINDING SITE III, inhibits translation of VGAM128 host target RNA into VGAM128 host target protein, herein designated VGAM HOST TARGET PROTEIN. VGAM host target protein is therefore outlined by a broken line.

It is appreciated that VGAM128 host target gene, herein designated VGAM HOST TARGET GENE, in fact represents a plurality of VGAM128 host target genes. The mRNA of each one of this plurality of VGAM128 host target genes comprises one or more host target binding sites, each having a nucleotide sequence which is at least partly complementary to VGAM128 RNA, herein designated VGAM RNA, and which when bound by VGAM128 RNA causes inhibition of translation of respective one or more VGAM128 host target proteins.

It is further appreciated by one skilled in the art that the mode of translational inhibition illustrated by FIG. 1 with specific reference to translational inhibition exerted by VGAM128 gene, herein designated VGAM GENE, on one or more VGAM128 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM128 is inhibition of expression of host target genes, as part of a novel viral mechanism of attacking a host. Accordingly, utilities of VGAM128 include diagnosis, prevention and treatment of viral infection by Vaccinia Virus. Specific functions, and accordingly utilities, of VGAM128 correlate with, and may be deduced from, the identity of the host target genes which VGAM128 binds and inhibits, and the function of these host target genes, as elaborated hereinbelow.

Nucleotide sequences of the VGAM128 precursor RNA, herein designated VGAM PRECURSOR RNA, and of the ‘diced’ VGAM128 RNA, herein designated VGAM RNA, and a schematic representation of the secondary folding of VGAM128 folded precursor RNA, herein designated VGAM FOLDED PRECURSOR RNA, of VGAM128 are further described hereinbelow with reference to Table 1.

Nucleotide sequences of host target binding sites, such as BINDING SITE-I, BINDING SITE-II and BINDING SITE-III of FIG. 1, found on VGAM128 host target RNA, and schematic representation of the complementarity of each of these host target binding sites to VGAM128 RNA, herein designated VGAM RNA, are described hereinbelow with reference to Table 2.

As mentioned hereinabove with reference to FIG. 1, a function of VGAM128 gene, herein designated VGAM is inhibition of expression of VGAM128 target genes. It is appreciated that specific functions, and accordingly utilities, of VGAM128 correlate with, and may be deduced from, the identity of the target genes which VGAM128 binds and inhibits, and the function of these target genes, as elaborated hereinbelow.

Sodium Channel, Nonvoltage-gated 1, Gamma (SCNN1G, Accession NM_{—}001039) is a VGAM128 host target gene. SCNN1G BINDING SITE is HOST TARGET binding site found in the 3′ untranslated region of mRNA encoded by SCNN1G, corresponding to a HOST TARGET binding site such as BINDING SITE I, BINDING SITE II or BINDING SITE III. Table 2 illustrates the complementarity of the nucleotide sequences of SCNN1G BINDING SITE, designated SEQ ID:793, to the nucleotide sequence of VGAM128 RNA, herein designated VGAM RNA, also designated SEQ ID:463.

A function of VGAM128 is therefore inhibition of Sodium Channel, Nonvoltage-gated 1, Gamma (SCNN1G, Accession NM_{—}001039). Accordingly, utilities of VGAM128 include diagnosis, prevention and treatment of diseases and clinical conditions associated with SCNN1G. YY1 Transcription Factor (YY1, Accession NM_{—}003403) is another VGAM128 host target gene. YY1 BINDING SITE is HOST TARGET binding site found in the 3′ untranslated region of mRNA encoded by YY1, corresponding to a HOST TARGET binding site such as BINDING SITE I, BINDING SITE II or BINDING SITE III. Table 2 illustrates the complementarity of the nucleotide sequences of YY1 BINDING SITE, designated SEQ ID: 1016, to the nucleotide sequence of VGAM128 RNA, herein designated VGAM RNA, also designated SEQ ID:463.

Another function of VGAM128 is therefore inhibition of YY1 Transcription Factor (YY1, Accession NM_{—}003403), a gene which is involved in transcriptional regulation and may play an important role in development and differentiation. Accordingly, utilities of VGAM128 include diagnosis, prevention and treatment of diseases and clinical conditions associated with YY1. The function of YY1 has been established by previous studies. Functionally, YY1 is a versatile factor, being a negative regulator in some systems and a positive regulator in others. In some systems, the function of YY1 as an activator or a repressor is specified by the presence of other proteins. By site-directed mutagenesis and overexpression of YY1 in human fibroblasts, Yan et al. (2002) showed that YY1, as well as HRY (OMIM Ref. No. 139605), functions as a transcriptional activator of acid alpha-glucosidase (GAA; 232300). In previous studies, Yan et al. (2001) had found that YY1, binding to the same element of the GAA gene in hepatoma cells, acts as a GAA transcription silencer. Oei and Shi (2001) noted that physical interaction had been reported between YY1 and poly(ADP-ribose) polymerase (PARP; 173870). PARP is a nuclear enzyme that catalyzes the synthesis of ADP-ribose polymers from NAD+, a function related to DNA repair and transcription. Oei and Shi (2001) found that overexpression of YY1 in HeLa cells resulted in intracellular accumulation of poly(ADP-ribose) and acceleration of DNA repair following damage with genotoxic agents, suggesting a functional as well as physical interaction between the proteins.

Full details of the abovementioned studies are described in the following publications, the disclosure of which are hereby incorporated by reference:
Oei, S. L.; Shi, Y.: Transcription factor Yin Yang 1 stimulates poly(ADP-ribosyl)ation and DNA repair. Biochem. Biophys. Res. Commun. 284: 450-454, 2001.; and
Yan, B.; Raben, N.; Plotz, P. H.: Hes-1, a known transcriptional repressor, acts as a transcriptional activator for the human acid alpha-glucosidase gene in human fibroblast cells. Bi.

Further studies establishing the function and utilities of YY1 are found in John Hopkins OMIM database record ID 600013, and in sited publications numbered 1906-1910, 842-84 and 1911-1912 listed in the bibliography section hereinbelow, which are also hereby incorporated by reference. FLJ21940 (Accession NM_{—}022828) is another VGAM128 host target gene. FLJ21940 BINDING SITE is HOST TARGET binding site found in the 3′ untranslated region of mRNA encoded by FLJ21940, corresponding to a HOST TARGET binding site such as BINDING SITE I, BINDING SITE II or BINDING SITE III. Table 2 illustrates the complementarity of the nucleotide sequences of FLJ21940 BINDING SITE, designated SEQ ID:2014, to the nucleotide sequence of VGAM128 RNA, herein designated VGAM RNA, also designated SEQ ID:463.

Another function of VGAM128 is therefore inhibition of FLJ21940 (Accession NM_{—}022828). Accordingly, utilities of VGAM128 include diagnosis, prevention and treatment of diseases and clinical conditions associated with FLJ21940. PRO0159 (Accession NM_{—}014118) is another VGAM128 host target gene. PRO0159 BINDING SITE is HOST TARGET binding site found in the 5′ untranslated region of mRNA encoded by PRO0159, corresponding to a HOST TARGET binding site such as BINDING SITE I, BINDING SITE II or BINDING SITE III. Table 2 illustrates the complementarity of the nucleotide sequences of PRO0159 BINDING SITE, designated SEQ ID: 1474, to the nucleotide sequence of VGAM128 RNA, herein designated VGAM RNA, also designated SEQ ID:463.

Another function of VGAM128 is therefore inhibition of PRO0159 (Accession NM_{—}014118). Accordingly, utilities of VGAM128 include diagnosis, prevention and treatment of diseases and clinical conditions associated with PRO0159. LOC200282 (Accession XM_{—}114184) is another VGAM128 host target gene. LOC200282 BINDING SITE is HOST TARGET binding site found in the 3′ untranslated region of mRNA encoded by LOC200282, corresponding to a HOST TARGET binding site such as BINDING SITE I, BINDING SITE II or BINDING SITE III. Table 2 illustrates the complementarity of the nucleotide sequences of LOC200282 BINDING SITE, designated SEQ ID:3448, to the nucleotide sequence of VGAM128 RNA, herein designated VGAM RNA, also designated SEQ ID:463.

Another function of VGAM128 is therefore inhibition of LOC200282 (Accession XM_{—}114184). Accordingly, utilities of VGAM128 include diagnosis, prevention and treatment of diseases and clinical conditions associated with LOC200282. FIG. 1 further provides a conceptual description of a novel bioinformatically detected viral gene of the present invention, referred to here as Viral Genomic Address Messenger 129 (VGAM129) viral gene, which modulates expression of respective host target genes thereof, the function and utility of which host target genes is known in the art.

VGAM129 is a novel bioinformatically detected regulatory, non protein coding, viral micro RNA (miRNA) gene. The method by which VGAM129 was detected is described hereinabove with reference to FIGS. 1-8.

VGAM129 gene, herein designated VGAM GENE, is a viral gene contained in the genome of Vaccinia Virus. VGAM129 host target gene, herein designated VGAM HOST TARGET GENE, is a human gene contained in the human genome.

VGAM129 gene encodes a VGAM129 precursor RNA, herein designated VGAM PRECURSOR RNA. Similar to other miRNA genes, and unlike most ordinary genes, VGAM129 precursor RNA does not encode a protein. A nucleotide sequence identical or highly similar to the nucleotide sequence of VGAM129 precursor RNA is designated SEQ ID:115, and is provided hereinbelow with reference to the sequence listing part. Nucleotide sequence SEQ ID:115 is located at position 57776 relative to the genome of Vaccinia Virus.

VGAM129 precursor RNA folds onto itself, forming VGAM129 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM129 folded precursor RNA into VGAM129 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM129 RNA is designated SEQ ID:464, and is provided hereinbelow with reference to the sequence listing part.

VGAM129 host target gene, herein designated VGAM HOST TARGET GENE, encodes a corresponding messenger RNA, VGAM129 host target RNA, herein designated VGAM HOST TARGET RNA. VGAM129 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM129 RNA, herein designated VGAM RNA, binds complementarily to one or more host target binding sites located in untranslated regions of VGAM129 host target RNA, herein designated VGAM HOST TARGET RNA. This complementary binding is due to the fact that the nucleotide sequence of VGAM129 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM129 RNA, herein designated VGAM RNA, may have a different number of host target binding sites in untranslated regions of a VGAM129 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM129 RNA, herein designated VGAM RNA, to host target binding sites on VGAM129 host target RNA, herein designated VGAM HOST TARGET RNA, such as BINDING SITE I, BINDING SITE II and BINDING SITE III, inhibits translation of VGAM129 host target RNA into VGAM129 host target protein, herein designated VGAM HOST TARGET PROTEIN. VGAM host target protein is therefore outlined by a broken line.

It is appreciated that VGAM129 host target gene, herein designated VGAM HOST TARGET GENE, in fact represents a plurality of VGAM129 host target genes. The mRNA of each one of this plurality of VGAM129 host target genes comprises one or more host target binding sites, each having a nucleotide sequence which is at least partly complementary to VGAM129 RNA, herein designated VGAM RNA, and which when bound by VGAM129 RNA causes inhibition of translation of respective one or more VGAM129 host target proteins.

It is further appreciated by one skilled in the art that the mode of translational inhibition illustrated by FIG. 1 with specific reference to translational inhibition exerted by VGAM129 gene, herein designated VGAM GENE, on one or more VGAM129 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM129 is inhibition of expression of host target genes, as part of a novel viral mechanism of attacking a host. Accordingly, utilities of VGAM129 include diagnosis, prevention and treatment of viral infection by Vaccinia Virus. Specific functions, and accordingly utilities, of VGAM129 correlate with, and may be deduced from, the identity of the host target genes which VGAM129 binds and inhibits, and the function of these host target genes, as elaborated hereinbelow.

Nucleotide sequences of the VGAM129 precursor RNA, herein designated VGAM PRECURSOR RNA, and of the ‘diced’ VGAM129 RNA, herein designated VGAM RNA, and a schematic representation of the secondary folding of VGAM129 folded precursor RNA, herein designated VGAM FOLDED PRECURSOR RNA, of VGAM129 are further described hereinbelow with reference to Table 1.

Nucleotide sequences of host target binding sites, such as BINDING SITE-I, BINDING SITE-II and BINDING SITE-III of FIG. 1, found on VGAM129 host target RNA, and schematic representation of the complementarity of each of these host target binding sites to VGAM129 RNA, herein designated VGAM RNA, are described hereinbelow with reference to Table 2.

As mentioned hereinabove with reference to FIG. 1, a function of VGAM129 gene, herein designated VGAM is inhibition of expression of VGAM129 target genes. It is appreciated that specific functions, and accordingly utilities, of VGAM129 correlate with, and may be deduced from, the identity of the target genes which VGAM129 binds and inhibits, and the function of these target genes, as elaborated hereinbelow.

Proto-cadherin 9 (PCDH9, Accession XM_{—}096054) is a VGAM129 host target gene. PCDH9 BINDING SITE is HOST TARGET binding site found in the 5′ untranslated region of mRNA encoded by PCDH9, corresponding to a HOST TARGET binding site such as BINDING SITE I, BINDING SITE II or BINDING SITE III. Table 2 illustrates the complementarity of the nucleotide sequences of PCDH9 BINDING SITE, designated SEQ ID:3262, to the nucleotide sequence of VGAM129 RNA, herein designated VGAM RNA, also designated SEQ ID:464.

A function of VGAM129 is therefore inhibition of Proto-cadherin 9 (PCDH9, Accession XM_{—}096054). Accordingly, utilities of VGAM129 include diagnosis, prevention and treatment of diseases and clinical conditions associated with PCDH9. ERO1-like (S. cerevisiae) (ERO1L, Accession NM_{—}014584) is another VGAM129 host target gene. ERO1L BINDING SITE is HOST TARGET binding site found in the 3′ untranslated region of mRNA encoded by ERO1L, corresponding to a HOST TARGET binding site such as BINDING SITE I, BINDING SITE II or BINDING SITE III. Table 2 illustrates the complementarity of the nucleotide sequences of ERO1L BINDING SITE, designated SEQ ID:1508, to the nucleotide sequence of VGAM129 RNA, herein designated VGAM RNA, also designated SEQ ID:464.

Another function of VGAM129 is therefore inhibition of ERO1-like (S. cerevisiae) (ERO1L, Accession NM_{—}014584). Accordingly, utilities of VGAM129 include diagnosis, prevention and treatment of diseases and clinical conditions associated with ERO1L. LOC253287 (Accession XM_{—}173745) is another VGAM129 host target gene. LOC253287 BINDING SITE is HOST TARGET binding site found in the 5′ untranslated region of mRNA encoded by LOC253287, corresponding to a HOST TARGET binding site such as BINDING SITE I, BINDING SITE II or BINDING SITE III. Table 2 illustrates the complementarity of the nucleotide sequences of LOC253287 BINDING SITE, designated SEQ ID:3740, to the nucleotide sequence of VGAM129 RNA, herein designated VGAM RNA, also designated SEQ ID:464.

Another function of VGAM129 is therefore inhibition of LOC253287 (Accession XM_{—}173745). Accordingly, utilities of VGAM129 include diagnosis, prevention and treatment of diseases and clinical conditions associated with LOC253287. LOC254268 (Accession XM_{—}170913) is another VGAM129 host target gene. LOC254268 BINDING SITE is HOST TARGET binding site found in the 5′ untranslated region of mRNA encoded by LOC254268, corresponding to a HOST TARGET binding site such as BINDING SITE I, BINDING SITE II or BINDING SITE III. Table 2 illustrates the complementarity of the nucleotide sequences of LOC254268 BINDING SITE, designated SEQ ID:3674, to the nucleotide sequence of VGAM129 RNA, herein designated VGAM RNA, also designated SEQ ID:464.

Another function of VGAM129 is therefore inhibition of LOC254268 (Accession XM_{—}170913). Accordingly, utilities of VGAM129 include diagnosis, prevention and treatment of diseases and clinical conditions associated with LOC254268. FIG. 1 further provides a conceptual description of a novel bioinformatically detected viral gene of the present invention, referred to here as Viral Genomic Address Messenger 130 (VGAM130) viral gene, which modulates expression of respective host target genes thereof, the function and utility of which host target genes is known in the art.

VGAM130 is a novel bioinformatically detected regulatory, non protein coding, viral micro RNA (miRNA) gene. The method by which VGAM130 was detected is described hereinabove with reference to FIGS. 1-8.

VGAM130 gene, herein designated VGAM GENE, is a viral gene contained in the genome of Vaccinia Virus. VGAM130 host target gene, herein designated VGAM HOST TARGET GENE, is a human gene contained in the human genome.

VGAM130 gene encodes a VGAM130 precursor RNA, herein designated VGAM PRECURSOR RNA. Similar to other miRNA genes, and unlike most ordinary genes, VGAM130 precursor RNA does not encode a protein. A nucleotide sequence identical or highly similar to the nucleotide sequence of VGAM130 precursor RNA is designated SEQ ID:116, and is provided hereinbelow with reference to the sequence listing part. Nucleotide sequence SEQ ID:116 is located at position 60133 relative to the genome of Vaccinia Virus.

VGAM130 precursor RNA folds onto itself, forming VGAM130 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM130 folded precursor RNA into VGAM130 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM130 RNA is designated SEQ ID:465, and is provided hereinbelow with reference to the sequence listing part.

VGAM130 host target gene, herein designated VGAM HOST TARGET GENE, encodes a corresponding messenger RNA, VGAM130 host target RNA, herein designated VGAM HOST TARGET RNA. VGAM130 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM130 RNA, herein designated VGAM RNA, binds complementarily to one or more host target binding sites located in untranslated regions of VGAM130 host target RNA, herein designated VGAM HOST TARGET RNA. This complementary binding is due to the fact that the nucleotide sequence of VGAM130 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM130 RNA, herein designated VGAM RNA, may have a different number of host target binding sites in untranslated regions of a VGAM130 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM130 RNA, herein designated VGAM RNA, to host target binding sites on VGAM130 host target RNA, herein designated VGAM HOST TARGET RNA, such as BINDING SITE I, BINDING SITE II and BINDING SITE III, inhibits translation of VGAM130 host target RNA into VGAM130 host target protein, herein designated VGAM HOST TARGET PROTEIN. VGAM host target protein is therefore outlined by a broken line.

It is appreciated that VGAM130 host target gene, herein designated VGAM HOST TARGET GENE, in fact represents a plurality of VGAM130 host target genes. The mRNA of each one of this plurality of VGAM130 host target genes comprises one or more host target binding sites, each having a nucleotide sequence which is at least partly complementary to VGAM130 RNA, herein designated VGAM RNA, and which when bound by VGAM130 RNA causes inhibition of translation of respective one or more VGAM130 host target proteins.

It is further appreciated by one skilled in the art that the mode of translational inhibition illustrated by FIG. 1 with specific reference to translational inhibition exerted by VGAM130 gene, herein designated VGAM GENE, on one or more VGAM130 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM130 is inhibition of expression of host target genes, as part of a novel viral mechanism of attacking a host. Accordingly, utilities of VGAM130 include diagnosis, prevention and treatment of viral infection by Vaccinia Virus. Specific functions, and accordingly utilities, of VGAM130 correlate with, and may be deduced from, the identity of the host target genes which VGAM130 binds and inhibits, and the function of these host target genes, as elaborated hereinbelow.

Nucleotide sequences of the VGAM130 precursor RNA, herein designated VGAM PRECURSOR RNA, and of the ‘diced’ VGAM130 RNA, herein designated VGAM RNA, and a schematic representation of the secondary folding of VGAM130 folded precursor RNA, herein designated VGAM FOLDED PRECURSOR RNA, of VGAM130 are further described hereinbelow with reference to Table 1.

Nucleotide sequences of host target binding sites, such as BINDING SITE-I, BINDING SITE-II and BINDING SITE-III of FIG. 1, found on VGAM130 host target RNA, and schematic representation of the complementarity of each of these host target binding sites to VGAM130 RNA, herein designated VGAM RNA, are described hereinbelow with reference to Table 2.

As mentioned hereinabove with reference to FIG. 1, a function of VGAM130 gene, herein designated VGAM is inhibition of expression of VGAM130 target genes. It is appreciated that specific functions, and accordingly utilities, of VGAM130 correlate with, and may be deduced from, the identity of the target genes which VGAM130 binds and inhibits, and the function of these target genes, as elaborated hereinbelow.

Adaptor-related Protein Complex 1, Beta 1 Subunit (AP1B1, Accession NM_{—}001127) is a VGAM130 host target gene. AP1B1 BINDING SITE is HOST TARGET binding site found in the 3′ untranslated region of mRNA encoded by AP1B1, corresponding to a HOST TARGET binding site such as BINDING SITE I, BINDING SITE II or BINDING SITE III. Table 2 illustrates the complementarity of the nucleotide sequences of AP1B1 BINDING SITE, designated SEQ ID:802, to the nucleotide sequence of VGAM130 RNA, herein designated VGAM RNA, also designated SEQ ID:465.

A function of VGAM130 is therefore inhibition of Adaptor-related Protein Complex 1, Beta 1 Subunit (AP1B1, Accession NM_{—}001127), a gene which plays a role in protein sorting in the late-golgi/trans-golgi network (tgn) and/or endosomes. Accordingly, utilities of VGAM130 include diagnosis, prevention and treatment of diseases and clinical conditions associated with AP1B1. The function of AP1B1 and its association with various diseases and clinical conditions, has been established by previous studies, as described hereinabove with reference to VGAM84. Bardet-Biedl Syndrome 2 (BBS2, Accession NM_{—}031885) is another VGAM130 host target gene. BBS2 BINDING SITE is HOST TARGET binding site found in the 5′ untranslated region of mRNA encoded by BBS2, corresponding to a HOST TARGET binding site such as BINDING SITE I, BINDING SITE II or BINDING SITE III. Table 2 illustrates the complementarity of the nucleotide sequences of BBS2 BINDING SITE, designated SEQ ID:2214, to the nucleotide sequence of VGAM130 RNA, herein designated VGAM RNA, also designated SEQ ID:465.

Another function of VGAM130 is therefore inhibition of Bardet-Biedl Syndrome 2 (BBS2, Accession NM_{—}031885). Accordingly, utilities of VGAM130 include diagnosis, prevention and treatment of diseases and clinical conditions associated with BBS2. Fyn-related Kinase (FRK, Accession NM_{—}002031) is another VGAM130 host target gene. FRK BINDING SITE is HOST TARGET binding site found in the 5′ untranslated region of mRNA encoded by FRK, corresponding to a HOST TARGET binding site such as BINDING SITE I, BINDING SITE II or BINDING SITE III. Table 2 illustrates the complementarity of the nucleotide sequences of FRK BINDING SITE, designated SEQ ID:890, to the nucleotide sequence of VGAM130 RNA, herein designated VGAM RNA, also designated SEQ ID:465.

Another function of VGAM130 is therefore inhibition of Fyn-related Kinase (FRK, Accession NM_{—}002031), a gene which binds pRb (RB1) during G1 and S phase and suppresses growth. Accordingly, utilities of VGAM130 include diagnosis, prevention and treatment of diseases and clinical conditions associated with FRK. The function of FRK has been established by previous studies. Tyrosine kinases are either expressed cytoplasmically, such as SRC (CSK; 124095), or as transmembrane receptors, such as growth factor receptors. They are involved in signal transduction and the regulation of cellular proliferation and have been linked to tumorigenesis through overexpression or mutation. Anneren et al. (2000) showed that expression of Gtk, the rodent homolog of FRK, in a rat pheochromocytoma cell line used as a model for neuronal cell differentiation induced nerve growth factor (see OMIM Ref. No. 162030)-independent neurite outgrowth and Rap1 (OMIM Ref. No. 605061) activation, probably through activation of the Crkll (OMIM Ref. No. 164762)-C3G (GRF2; 600303) pathway.

Full details of the abovementioned studies are described in the following publications, the disclosure of which are hereby incorporated by reference:
Anneren, C.; Reedquist, K. A.; Bos, J. L.; Welsh, M.: GTK, a Src-related tyrosine kinase, induces nerve growth factor-independent neurite outgrowth in PC12 cells through activation of the Rapl pathway: relationship to Shb tyrosine phosphorylation and elevated levels of focal adhesion kinase. J. Biol. Chem. 275: 29153-29161, 2000.; and
Cance, W. G.; Craven, R. J.; Bergman, M.; Xu, L.; Alitalo, K.; Liu, E. T.: Rak, a novel nuclear tyrosine kinase expressed in epithelial cells. Cell Growth Differ. 5: 1347-1355, 1994.

Further studies establishing the function and utilities of FRK are found in John Hopkins OMIM database record ID 606573, and in sited publications numbered 1409-1413 listed in the bibliography section hereinbelow, which are also hereby incorporated by reference. Heat Shock 60 kDa Protein 1 (chaperonin) (HSPD1, Accession XM_{—}012182) is another VGAM130 host target gene. HSPD1 BINDING SITE is HOST TARGET binding site found in the 3′ untranslated region of mRNA encoded by HSPD1, corresponding to a HOST TARGET binding site such as BINDING SITE I, BINDING SITE II or BINDING SITE III. Table 2 illustrates the complementarity of the nucleotide sequences of HSPD1 BINDING SITE, designated SEQ ID:2560, to the nucleotide sequence of VGAM130 RNA, herein designated VGAM RNA, also designated SEQ ID:465.

Another function of VGAM130 is therefore inhibition of Heat Shock 60 kDa Protein 1 (chaperonin) (HSPD1, Accession XM_{—}012182), a gene which is implicated in mitochondrial protein import and macromolecular assembly. may facilitate the correct folding of imported proteins. may also prevent misfolding and promote the refolding and proper assembly of unfolded polypeptides generated under stress conditions in the mitochondrial matrix. Accordingly, utilities of VGAM130 include diagnosis, prevention and treatment of diseases and clinical conditions associated with HSPD1. The function of HSPD1 has been established by previous studies. Hereditary spastic paraplegia (HSP) represents a clinically and genetically heterogeneous group of neurodegenerative disorders that are characterized by progressive spasticity and weakness of the lower limbs. Seventeen different loci had been mapped, and the corresponding genes for 5 of these had been cloned and identified. Two of the 5 gene products-paraplegin (SPG7; 602783) and spastin (SPG4; 182601)—feature AAA+ domains and are predicted to possess chaperone activity. Paraplegin is the human homolog of a yeast protease/chaperone that is involved in mitochondrial protein quality control. The HSP60 gene maps to the same region, namely 2q33.1, as does spastic paraplegia-13 (SPG13; 605280), as determined by Fontaine et al. (2000). Speculating that the mitochondrial chaperonin HSP60 or its co-chaperonin HSP10 (OMIM Ref. No. 600141), which maps to the same region, might be the site of mutation(s) causing SPG13, Hansen et al. (2002) sequenced HSP60 in 2 affected members of the family with SPG13. They found that both were heterozygous for a G-to-A variation at position 292 of the HSP60 cDNA, resulting in the substitution of a valine at position 72 in the mature HSP60 by isoleucine (V72I). Studies in E. coli indicated that the V72I mutant protein is functionally incapacitated. The authors suggested that SPG4, SPG7, and SPG13 can be referred to as chaperonopathies. Azem et al. (1994) performed chemical cross-linking and electron microscopy studies on bacterial chaperonins GroEL and GroES to determine how they interact with unfolded proteins. GroEL is an oligomer of 14 identical 57.3-kD subunits, with a structure of 2 stacked heptameric rings arranged around a 2-fold axis of symmetry (Saibil et al., 1991). It appears as a hollow cylinder. In the presence of ATP, 2 GroES (see OMIM Ref. No. 600141) rings (each made of 7 identical 10.4-kD subunits) can successively bind a single GroEL core to make a functional symmetric heterodimer. Although the central core of GroEL is obstructed by the 2 GroES rings at each end, this heterodimer can stably bind and assist the refolding of the RuBisCo enzyme. While binding was thought to occur in the central cavity, these data indicate that unfolded proteins may bind and fold on the external envelope of some chaperonins (Azem et al., 1994). Schmidt et al. (1994) suggested that the symmetric chaperonin complex is functionally significant because complete folding of a normative substrate protein in the presence of GroEL and GroES occurs only in the presence of ATP, and not with ADP. Chaperonin-assisted folding occurs by a catalytic cycle in which one ATP is hydrolyzed by one ring of GroEL in a quantized manner with each turnover. Todd et al. (1994) proposed a unifying model for chaperonin-facilitated protein folding based on successive rounds of binding and release, and partitioning between committed and kinetically trapped intermediates.

Full details of the abovementioned studies are described in the following publications, the disclosure of which are hereby incorporated by reference:
Fontaine, B.; Davoine, C.-S.; Durr, A.; Paternotte, C.; Feki, I.; Weissenbach, J.; Hazan, J.; Brice, A.: A new locus for autosomal dominant pure spastic paraplegia, on chromosome 2q24-q34. Am. J. Hum. Genet. 66: 702-707, 2000.; and
Hansen, J. J.; Durr, A.; Cournu-Rebeix, I.; Georgopoulos, C.; Ang, D.; Nielsen, M. N.; Davoine, C.-S.; Brice, A.; Fontaine, B.; Gregersen, N.; Bross, P.: Hereditary spastic paraplegia SPG.

Further studies establishing the function and utilities of HSPD1 are found in John Hopkins OMIM database record ID 118190, and in sited publications numbered 193-196, 2446-19 and 2447 listed in the bibliography section hereinbelow, which are also hereby incorporated by reference. Interleukin 20 Receptor, Alpha (IL20RA, Accession NM_{—}014432) is another VGAM130 host target gene. IL20RA BINDING SITE is HOST TARGET binding site found in the 3′ untranslated region of mRNA encoded by IL20RA, corresponding to a HOST TARGET binding site such as BINDING SITE I, BINDING SITE II or BINDING SITE III. Table 2 illustrates the complementarity of the nucleotide sequences of IL20RA BINDING SITE, designated SEQ ID:1501, to the nucleotide sequence of VGAM130 RNA, herein designated VGAM RNA, also designated SEQ ID:465.

Another function of VGAM130 is therefore inhibition of Interleukin 20 Receptor, Alpha (IL20RA, Accession NM_{—}014432), a gene which is the receptor for interleukin-20. Accordingly, utilities of VGAM130 include diagnosis, prevention and treatment of diseases and clinical conditions associated with IL20RA. The function of IL20RA has been established by previous studies. Blumberg et al. (2001) identified the receptor for interleukin-20 (IL20; 605619) as a heterodimer of 2 orphan class II cytokine receptor subunits, IL20RA, also called ZCYTOR7, and IL20RB (OMIM Ref. No. 605621), also called DIRS1. Binding assays using radiolabeled ligand demonstrated that IL20 bound to BHK transfectants expressing both IL20RA and IL20RB, but not to untransfected cells nor to transfectants expressing either receptor subunit alone. Binding of (125)I-labeled IL20 was eliminated in the presence of 100-fold excess of unlabeled IL20 but not in the presence of 100-fold excess of the unrelated cytokine, IL21 (OMIM Ref. No. 605384). The binding data revealed 88,000 IL20 receptors per cell, with a binding affinity of approximately 1.5 nM. Both receptor subunits were expressed in skin and were dramatically up-regulated in psoriatic skin. Scott (2001) mapped the IL20RA gene to 6q23 based on sequence similarity between the IL20RA sequence (GenBank AF184971) and a genomic contig (GenBank NT_{—}025741.1).

Full details of the abovementioned studies are described in the following publications, the disclosure of which are hereby incorporated by reference:
Blumberg, H.; Conklin, D.; Xu, W.; Grossmann, A.; Brender, T.; Carollo, S.; Eagan, M.; Foster, D.; Haldeman, B. A.; Hammond, A.; Haugen, H.; Jelinek, L.; and 14 others: Interleukin 20: discovery, receptor identification, and role in epidermal function. Cell 104: 9-19, 2001.; and
Scott, A. F.: Personal Communication. Baltimore, Md., Mar. 13, 2001.

Further studies establishing the function and utilities of IL20RA are found in John Hopkins OMIM database record ID 605620, and in sited publications numbered 99 and 1611 listed in the bibliography section hereinbelow, which are also hereby incorporated by reference. Laminin, Gamma 3 (LAMC3, Accession NM_{—}006059) is another VGAM130 host target gene. LAMC3 BINDING SITE is HOST TARGET binding site found in the 3′ untranslated region of mRNA encoded by LAMC3, corresponding to a HOST TARGET binding site such as BINDING SITE I, BINDING SITE II or BINDING SITE III. Table 2 illustrates the complementarity of the nucleotide sequences of LAMC3 BINDING SITE, designated SEQ ID:1269, to the nucleotide sequence of VGAM130 RNA, herein designated VGAM RNA, also designated SEQ ID:465.

Another function of VGAM130 is therefore inhibition of Laminin, Gamma 3 (LAMC3, Accession NM_{—}006059). Accordingly, utilities of VGAM130 include diagnosis, prevention and treatment of diseases and clinical conditions associated with LAMC3. Mesoderm Specific Transcript Homolog (mouse) (MEST, Accession XM_{—}046001) is another VGAM130 host target gene. MEST BINDING SITE is HOST TARGET binding site found in the 3′ untranslated region of mRNA encoded by MEST, corresponding to a HOST TARGET binding site such as BINDING SITE I, BINDING SITE II or BINDING SITE III. Table 2 illustrates the complementarity of the nucleotide sequences of MEST BINDING SITE, designated SEQ ID:2872, to the nucleotide sequence of VGAM130 RNA, herein designated VGAM RNA, also designated SEQ ID:465.

Another function of VGAM130 is therefore inhibition of Mesoderm Specific Transcript Homolog (mouse) (MEST, Accession XM_{—}046001), a gene which appears to be required for the appropriate immediate response of females to their pups. Accordingly, utilities of VGAM130 include diagnosis, prevention and treatment of diseases and clinical conditions associated with MEST. The function of MEST has been established by previous studies. The Mest gene maps to an imprinted region of mouse chromosome 6 and is expressed monoallelically from the paternal allele. When the null allele is paternally transmitted, the offspring exhibits severe intrauterine growth retardation. Uniparental disomy of mouse chromosome 6 is associated with a similar phenotype, presumably as a result of lack of expression of the Mest gene (Ferguson-Smith et al., 1991). The human homolog, MEST, maps to 7q31.3, within a region of conserved synteny corresponding to mouse chromosome 6, and is monoallelically expressed from the paternal allele in a wide variety of tissues during prenatal and postnatal development. Uniparental disomy of chromosome 7 in humans is associated with phenotypic features of Silver-Russell syndrome (SRS; 180860), a heterogeneous disorder characterized by intrauterine and postnatal growth retardation, with or without additional dysmorphic features. Kotzot et al. (1995) predicted the presence of at least one maternally repressed gene on human chromosome 7, because they found maternal uniparental disomy for this chromosome in 4 of 35 patients with SRS. Nishita et al. (1996) suggested that MEST, the first imprinted gene to be identified on chromosome 7, is involved in the causation of this syndrome. Riesewijk et al. (1998) performed a mutation screen of the PEG1/MEST gene in 49 patients with SRS and 9 patients with primordial growth retardation (PGR). As background for this, they determined the complete genomic structure of the MEST gene, which comprises 12 exons. Apart from 1 silent mutation and 2 novel polymorphisms, nucleotide changes were not detected in any of the SRS or PGR patients. Moreover, methylation patterns of the 5-prime region of PEG1/MEST were found to be normal in 35 SRS and 9 PGR patients and different from the pattern seen in patients with maternal uniparental disomy 7. Kobayashi et al. (2001) presented findings indicating that PEG1/MEST can be excluded as a major determinant of SRS. In a screening of 15 SRS patients, no aberrant expression patterns of 2 splice variants were detected in lymphocytes. Direct sequence analysis failed to detect any mutations in the coding region of isoform-1, which the authors called alpha, and there were no significant mutations in the 5-prime flanking upstream region containing the predicted promoter and the genomic region that is highly conserved between human and mouse. Differential methylation patterns of the CpG islands for the alpha isoform were normally maintained and resulted in the same patterns as in normal controls, suggesting that there was no loss of imprinting.

Full details of the abovementioned studies are described in the following publications, the disclosure of which are hereby incorporated by reference:
Nishita, Y.; Yoshida, I.; Sado, T.; Takagi, N.: Genomic imprinting and chromosomal localization of the human MEST gene. Genomics 36: 539-542, 1996.; and
Kobayashi, S.; Uemura, H.; Kohda, T.; Nagai, T.; Chinen, Y.; Naritomi, K.; Kinoshita, E.; Ohashi, H.; Imaizumi, K.; Tsukahara, M.; Sugio, Y.; Tonoki, H.; Kishino, T.; Tanaka, T.; Yamada, M.

Further studies establishing the function and utilities of MEST are found in John Hopkins OMIM database record ID 601029, and in sited publications numbered 949, 2241-2242, 2093, 2243-2246, 234 and 2438-2440 listed in the bibliography section hereinbelow, which are also hereby incorporated by reference. Antigen Identified By Monoclonal Antibody Ki-67 (MKI67, Accession NM_{—}002417) is another VGAM130 host target gene. MKI67 BINDING SITE is HOST TARGET binding site found in the 3′ untranslated region of mRNA encoded by MKI67, corresponding to a HOST TARGET binding site such as BINDING SITE I, BINDING SITE II or BINDING SITE III. Table 2 illustrates the complementarity of the nucleotide sequences of MKI67 BINDING SITE, designated SEQ ID:927, to the nucleotide sequence of VGAM130 RNA, herein designated VGAM RNA, also designated SEQ ID:465.

Another function of VGAM130 is therefore inhibition of Antigen Identified By Monoclonal Antibody Ki-67 (MKI67, Accession NM_{—}002417), a gene which thought to be required for maintaining cell proliferation. Accordingly, utilities of VGAM130 include diagnosis, prevention and treatment of diseases and clinical conditions associated with MKI67. The function of MKI67 has been established by previous studies. Ki-67 is a commercially available monoclonal antibody that reacts with a nuclear antigen expressed in proliferating cells but not in quiescent cells. Expression of this antigen occurs preferentially during late G1, S, G2, and M phases of the cell cycle, while in cells in G0 phase the antigen cannot be detected. Consequently, the antibody is used in tumor pathology to detect proliferating cells in neoplastic diseases. In cultured cells an antigen in the nucleolus of interphase cells stains with Ki-67, which, furthermore, reacts with an interchromatinous network during mitosis. From study of a panel of human-rodent somatic cell hybrids, Schonk et al. (1989) demonstrated that a gene involved in the expression of the antigen is located on chromosome 10. By in situ hybridization, Fonatsch et al. (1991) regionalized the MKI67 gene to 10q25-qter. By FISH, Traut et al. (1998) mapped the mouse Mki67 gene to chromosome 7F3-F5. By immunoscreening a cDNA expression library, followed by RT-PCR and 5-prime and 3-prime RACE, Schluter et al. (1993) isolated 2 cDNAs encoding isoforms of Ki-67. Genomic sequence analysis determined that the Ki-67 gene contains 15 exons. The Ki-67 repeat region, within which there is a 22-amino acid Ki-67 motif, is encoded by exon 13. The shorter isoform lacks exon 7. Northern blot analysis revealed multiple transcripts ranging from approximately 8.9 to 12.5 kb in proliferating but not quiescent cells. Immunoblot analysis showed expression of 320- and 359-kD proteins. Sequence analysis predicted that the short-lived 2,896- and 3,256-amino acid protein isoforms contain potential nuclear targeting signals, over 200 potential phosphorylation sites, 19 N-myristoylation sites, 3 amidation sites, and numerous PEST sites. Antisense oligonucleotides inhibited cellular proliferation in a dose-dependent manner, suggesting that Ki-67 protein expression may be an absolute requirement for cell proliferation.

Full details of the abovementioned studies are described in the following publications, the disclosure of which are hereby incorporated by reference:
Schluter, C.; Duchrow, M.; Wohlenberg, C.; Becker, M. H. G; Key, G.; Flad, H.-D.; Gerdes, J.: The cell proliferation-associated antigen of antibody Ki-67: a very large, ubiquitous nuclear protein with numerous repeated elements, representing a new kind of cell cycle-maintaining proteins. J. Cell. Biol. 123: 513-522, 1993.; and
Traut, W.; Scholzen, T.; Winking, H.; Kubbutat, M. H. G.; Gerdes, J.: Assignment of the murine Ki-67 gene (Mki67) to chromosome band 7F3-F5 by in situ hybridization. Cytogenet. Cell Ge.

Further studies establishing the function and utilities of MKI67 are found in John Hopkins OMIM database record ID 176741, and in sited publications numbered 384-387 listed in the bibliography section hereinbelow, which are also hereby incorporated by reference. Matrix Metalloproteinase 19 (MMP19, Accession NM_{—}022790) is another VGAM130 host target gene. MMP19 BINDING SITE1 and MMP19 BINDING SITE2 are HOST TARGET binding sites found in untranslated regions of mRNA encoded by MMP19, corresponding to HOST TARGET binding sites such as BINDING SITE I, BINDING SITE II or BINDING SITE III. Table 2 illustrates the complementarity of the nucleotide sequences of MMP19 BINDING SITE1 and MMP19 BINDING SITE2, designated SEQ ID:2011 and SEQ ID:2012 respectively, to the nucleotide sequence of VGAM130 RNA, herein designated VGAM RNA, also designated SEQ ID:465.

Another function of VGAM130 is therefore inhibition of Matrix Metalloproteinase 19 (MMP19, Accession NM_{—}022790). Accordingly, utilities of VGAM130 include diagnosis, prevention and treatment of diseases and clinical conditions associated with MMP19. Proteasome (prosome, macropain) 26S Subunit, Non-ATPase, 9 (PSMD9, Accession NM_{—}002813) is another VGAM130 host target gene. PSMD9 BINDING SITE is HOST TARGET binding site found in the 3′ untranslated region of mRNA encoded by PSMD9, corresponding to a HOST TARGET binding site such as BINDING SITE I, BINDING SITE II or BINDING SITE III. Table 2 illustrates the complementarity of the nucleotide sequences of PSMD9 BINDING SITE, designated SEQ ID:957, to the nucleotide sequence of VGAM130 RNA, herein designated VGAM RNA, also designated SEQ ID:465.

Another function of VGAM130 is therefore inhibition of Proteasome (prosome, macropain) 26S Subunit, Non-ATPase, 9 (PSMD9, Accession NM_{—}002813), a gene which acts as a regulatory subunit of the 26 proteasome. Accordingly, utilities of VGAM130 include diagnosis, prevention and treatment of diseases and clinical conditions associated with PSMD9. The function of PSMD9 has been established by previous studies. The 26S proteasome is a eukaryotic ATP-dependent protease that selectively degrades intracellular target proteins that are modified by the covalent attachment of ubiquitin. It is composed of a central catalytic 20S proteasome, which consists of a family of small proteins, and 2 large regulatory modules, named PA700, which consist of approximately 20 heterogeneous proteins. A proteasomal modulator complex, composed of p27, p42, and p50 subunits, stimulates the association of the 20S proteasome with PA700 to form the active 26S proteasome. Watanabe et al. (1998) cloned 2 distinct human brain cDNAs encoding p27, or PSMD9. Compared with the longer cDNA, the shorter cDNA has a 65-bp deletion near the 3-prime region that results in a new inframe termination codon farther downstream. The longer cDNA encodes a deduced 209-amino acid protein with a calculated molecular mass of 22,764 Da. The shorter cDNA encodes a deduced 223-amino acid protein with a calculated molecular mass of 24,652 Da. The longer PSMD9 protein exhibits 36% sequence identity with an S. cerevisiae protein, which the authors named NAS2 for ‘non-ATPase subunit 2,’ and 31.9% identity with a C. elegans protein. Disruption of the yeast NAS2 gene did not affect cell viability or proliferation. Watanabe et al. (1998) demonstrated that the PSMD9 protein, along with the ATPase components TBP1 (PSMC3; 186852) and p42 (PSMC6; 602708), associated with both the modulator complex and the 26S proteasome complex. Northern blot analysis detected an approximately 1.3-kb PSMD9 transcript in all tissues examined, with highest levels in liver and kidney. E12 and E47 (see OMIM Ref. No. TCF3; 147141), members of the ubiquitous E2A protein family, function with basic helix-loop-helix (bHLH) proteins to bind and transactivate promoters via conserved sequence elements known as E boxes. By yeast 2-hybrid screening of a rat insulinoma cell cDNA library using the bHLH domain-containing C terminus of E12 as bait, Thomas et al. (1999) obtained a cDNA encoding rat Bridge-1. Sequence analysis predicted that the 222-amino acid Bridge-1 protein shares 98% amino acid similarity with human PSMD9 over the first 184 amino acids but diverges in the C terminus Bridge-1 contains a PDZ-like domain from amino acids 138 to 178, forming 3 beta sheets and 2 alpha helices. SDS-PAGE analysis showed that Bridge-1 is expressed as a 28-kD protein, close to the deduced value of 25 kD. Using Bridge-1 cDNA as probe, Northern blot analysis detected a 1.0-kb transcript in all rat and human tissues tested, with highest expression in pancreas, testis, kidney, and liver. Immunocytochemistry assessment demonstrated predominant nuclear localization of Bridge-1, with lower levels in cytoplasm. Immunoprecipitation analysis determined that anti-Bridge-1 coimmunoprecipitates E12 or E12 and E47 through their C-terminal bHLH domains, but only in the presence of the PDZ domain of Bridge-1. CAT assays indicated that Bridge-1 together with E12 or E47 coactivates insulin (OMIM Ref. No. 176730) promoter elements.

Full details of the abovementioned studies are described in the following publications, the disclosure of which are hereby incorporated by reference:
Thomas, M. K.; Yao, K.-M.; Tenser, M. S.; Wong, G. G.; Habener, J. F.: Bridge-1, a novel PDZ-domain coactivator of E2A-mediated regulation of insulin gene transcription. Molec. Cell. Biol. 19: 8492-8504, 1999.; and
Watanabe, T. K.; Saito, A.; Suzuki, M.; Fujiwara, T.; Takahashi, E.; Slaughter, C. A.; DeMartino, G. N.; Hendil, K. B.; Chung, C. H.; Tanahashi, N.; Tanaka, K.: cDNA cloning and chara.

Further studies establishing the function and utilities of PSMD9 are found in John Hopkins OMIM database record ID 603146, and in sited publications numbered 1232-1233 listed in the bibliography section hereinbelow, which are also hereby incorporated by reference. SH3-domain GRB2-like 2 (SH3GL2, Accession NM_{—}003026) is another VGAM130 host target gene. SH3GL2 BINDING SITE is HOST TARGET binding site found in the 3′ untranslated region of mRNA encoded by SH3GL2, corresponding to a HOST TARGET binding site such as BINDING SITE I, BINDING SITE II or BINDING SITE III. Table 2 illustrates the complementarity of the nucleotide sequences of SH3GL2 BINDING SITE, designated SEQ ID:984, to the nucleotide sequence of VGAM130 RNA, herein designated VGAM RNA, also designated SEQ ID:465.

Another function of VGAM130 is therefore inhibition of SH3-domain GRB2-like 2 (SH3GL2, Accession NM_{—}003026), a gene which plays a role in synaptic vesicle recycling, in particular in clathrin-mediated vesicle endocytosis. Accordingly, utilities of VGAM130 include diagnosis, prevention and treatment of diseases and clinical conditions associated with SH3GL2. The function of SH3GL2 and its association with various diseases and clinical conditions, has been established by previous studies, as described hereinabove with reference to VGAM84. Spectrin, Alpha, Non-erythrocytic 1 (alpha-fodrin) (SPTAN1, Accession NM_{—}003127) is another VGAM130 host target gene. SPTAN1 BINDING SITE is HOST TARGET binding site found in the 3′ untranslated region of mRNA encoded by SPTAN1, corresponding to a HOST TARGET binding site such as BINDING SITE I, BINDING SITE II or BINDING SITE III. Table 2 illustrates the complementarity of the nucleotide sequences of SPTAN1 BINDING SITE, designated SEQ ID:994, to the nucleotide sequence of VGAM130 RNA, herein designated VGAM RNA, also designated SEQ ID:465.

Another function of VGAM130 is therefore inhibition of Spectrin, Alpha, Non-erythrocytic 1 (alpha-fodrin) (SPTAN1, Accession NM_{—}003127). Accordingly, utilities of VGAM130 include diagnosis, prevention and treatment of diseases and clinical conditions associated with SPTAN1. Synaptogyrin 1 (SYNGR1, Accession NM_{—}004711) is another VGAM130 host target gene. SYNGR1 BINDING SITE is HOST TARGET binding site found in the 3′ untranslated region of mRNA encoded by SYNGR1, corresponding to a HOST TARGET binding site such as BINDING SITE I, BINDING SITE II or BINDING SITE III. Table 2 illustrates the complementarity of the nucleotide sequences of SYNGR1 BINDING SITE, designated SEQ ID:1146, to the nucleotide sequence of VGAM130 RNA, herein designated VGAM RNA, also designated SEQ ID:465.

Another function of VGAM130 is therefore inhibition of Synaptogyrin 1 (SYNGR1, Accession NM_{—}004711), a gene which belongs to transmembrane synaptic vesicle protein and may function in membrane recycling. Accordingly, utilities of VGAM130 include diagnosis, prevention and treatment of diseases and clinical conditions associated with SYNGR1. The function of SYNGR1 and its association with various diseases and clinical conditions, has been established by previous studies, as described hereinabove with reference to VGAM97. Tripartite Motif-containing 9 (TRIM9, Accession NM_{—}052978) is another VGAM130 host target gene. TRIM9 BINDING SITE is HOST TARGET binding site found in the 3′ untranslated region of mRNA encoded by TRIM9, corresponding to a HOST TARGET binding site such as BINDING SITE I, BINDING SITE II or BINDING SITE III. Table 2 illustrates the complementarity of the nucleotide sequences of TRIM9 BINDING SITE, designated SEQ ID:2353, to the nucleotide sequence of VGAM130 RNA, herein designated VGAM RNA, also designated SEQ ID:465.

Another function of VGAM130 is therefore inhibition of Tripartite Motif-containing 9 (TRIM9, Accession NM_{—}052978), a gene which may function as a positive regulator for mannosylphosphate transferase and is required to mediate mannosylphosphate transfer in both the core and outer chain portions of n-linked. oligosaccharides. Accordingly, utilities of VGAM130 include diagnosis, prevention and treatment of diseases and clinical conditions associated with TRIM9. The function of TRIM9 and its association with various diseases and clinical conditions, has been established by previous studies, as described hereinabove with reference to VGAM101. Transient Receptor Potential Cation Channel, Sub family M, Member 2 (TRPM2, Accession NM_{—}003307) is another VGAM130 host target gene. TRPM2 BINDING SITE is HOST TARGET binding site found in the 5′ untranslated region of mRNA encoded by TRPM2, corresponding to a HOST TARGET binding site such as BINDING SITE I, BINDING SITE II or BINDING SITE III. Table 2 illustrates the complementarity of the nucleotide sequences of TRPM2 BINDING SITE, designated SEQ ID:1011, to the nucleotide sequence of VGAM130 RNA, herein designated VGAM RNA, also designated SEQ ID:465.

Another function of VGAM130 is therefore inhibition of Transient Receptor Potential Cation Channel, Sub family M, Member 2 (TRPM2, Accession NM_{—}003307), a gene which may be a calcium channel. Accordingly, utilities of VGAM130 include diagnosis, prevention and treatment of diseases and clinical conditions associated with TRPM2. The function of TRPM2 has been established by previous studies. By Southern analysis of somatic cell hybrids, Purrello et al. (1991) concluded that a single gene is responsible for the multiple functions of sulfated glycoprotein-2 and that the SGP2 gene is located on human chromosome 8. Slawin et al. (1990) also mapped SGP2 to chromosome 8 by Southern analysis of hamster-human hybrid cell lines. Likewise, To be et al. (1991) mapped the CLI gene to human chromosome 8 by spot blot hybridization of flow-sorted chromosomes using a cDNA probe. Dietzsch et al. (1992) regionalized the gene to 8p21-p12 by isotopic in situ hybridization. Using RFLVs (restriction fragment length variations) for interspecies linkage analysis, Birkenmeier et al. (1993) demonstrated that the homologous gene is located on mouse chromosome 14. By isolating and characterizing 3 partially overlapping cosmid clones, Fink et al. (1993) established the complete physical map of the clusterin gene which spans about 20 kb. By fluorescence in situ hybridization, they showed that CLI is located on 8p21, proximal to the lipoprotein lipase gene (OMIM Ref. No. 238600). They cited information suggesting that the CLI gene may be a candidate gene determining susceptibility to atherosclerosis. Animal model experiments lend further support to the function of TRPM2. Following neonatal hypoxic-ischemic brain injury in mice (a model of cerebral palsy), there is evidence of apoptotic changes such as activation of neuronal caspase-3 (OMIM Ref. No. 600636), as well as an accumulation of clusterin in dying neurons. Han et al. (2001) generated mice deficient in clusterin by targeted disruption. Clusterin −/− mice had 50% less brain injury following neonatal hypoxia-ischemia. The absence of clusterin had no effect on caspase-3 activation, and clusterin accumulation and caspase-3 activation did not colocalize to the same cells. Studies with cultured cortical neurons demonstrated that exogenous purified astrocyte-secreted clusterin exacerbated oxygen/glucose-deprivation-induced necrotic death. Han et al. (2001) concluded that clusterin may be a therapeutic target to modulate noncaspase-dependent neuronal death following acute brain injury.

It is appreciated that the abovementioned animal model for TRPM2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Fink, T. M.; Zimmer, M.; Tschopp, J.; Etienne, J.; Jenne, D. E.; Lichter, P.: Human clusterin (CLI) maps to 8p21 in proximity to the lipoprotein lipase (LPL) gene. Genomics 16: 526-528, 1993.; and
Han, B. H.; DeMattos, R. B.; Dugan, L. L.; Kim-Han, J. S.; Brendza, R. P.; Fryer, J. D.; Kierson, M.; Cirrito, J.; Quick, K.; Harmony, J. A. K.; Aronow, B. J.; Holtzman, D. M.: Clusterin c.

Further studies establishing the function and utilities of TRPM2 are found in John Hopkins OMIM database record ID 185430, and in sited publications numbered 184-186, 108-118, 599-12 and 290-292 listed in the bibliography section hereinbelow, which are also hereby incorporated by reference. ATP Synthase Mitochondrial F1 Complex Assembly Factor 1 (ATPAF1, Accession XM_{—}027313) is another VGAM130 host target gene. ATPAF1 BINDING SITE is HOST TARGET binding site found in the 3′ untranslated region of mRNA encoded by ATPAF1, corresponding to a HOST TARGET binding site such as BINDING SITE I, BINDING SITE II or BINDING SITE III. Table 2 illustrates the complementarity of the nucleotide sequences of ATPAF1 BINDING SITE, designated SEQ ID:2582, to the nucleotide sequence of VGAM130 RNA, herein designated VGAM RNA, also designated SEQ ID:465.

Another function of VGAM130 is therefore inhibition of ATP Synthase Mitochondrial F1 Complex Assembly Factor 1 (ATPAF1, Accession XM_{—}027313). Accordingly, utilities of VGAM130 include diagnosis, prevention and treatment of diseases and clinical conditions associated with ATPAF1. Centaurin, Gamma 2 (CENTG2, Accession NM_{—}014914) is another VGAM130 host target gene. CENTG2 BINDING SITE is HOST TARGET binding site found in the 3′ untranslated region of mRNA encoded by CENTG2, corresponding to a HOST TARGET binding site such as BINDING SITE I, BINDING SITE II or BINDING SITE III. Table 2 illustrates the complementarity of the nucleotide sequences of CENTG2 BINDING SITE, designated SEQ ID:1587, to the nucleotide sequence of VGAM130 RNA, herein designated VGAM RNA, also designated SEQ ID:465.

Another function of VGAM130 is therefore inhibition of Centaurin, Gamma 2 (CENTG2, Accession NM_{—}014914). Accordingly, utilities of VGAM130 include diagnosis, prevention and treatment of diseases and clinical conditions associated with CENTG2. Collagen, Type XII, Alpha 1 (COL12A1, Accession NM_{—}080645) is another VGAM130 host target gene. COL12A1 BINDING SITE1 and COL12A1 BINDING SITE2 are HOST TARGET binding sites found in untranslated regions of mRNA encoded by COL12A1, corresponding to HOST TARGET binding sites such as BINDING SITE I, BINDING SITE II or BINDING SITE III. Table 2 illustrates the complementarity of the nucleotide sequences of COL12A1 BINDING SITE1 and COL12A1 BINDING SITE2, designated SEQ ID:2381 and SEQ ID:1103 respectively, to the nucleotide sequence of VGAM130 RNA, herein designated VGAM RNA, also designated SEQ ID:465.

Another function of VGAM130 is therefore inhibition of Collagen, Type XII, Alpha 1 (COL12A1, Accession NM_{—}080645). Accordingly, utilities of VGAM130 include diagnosis, prevention and treatment of diseases and clinical conditions associated with COL12A1. DKFZp566H0824 (Accession NM_{—}017535) is another VGAM130 host target gene. DKFZp566H0824 BINDING SITE is HOST TARGET binding site found in the 3′ untranslated region of mRNA encoded by DKFZp566H0824, corresponding to a HOST TARGET binding site such as BINDING SITE I, BINDING SITE II or BINDING SITE III. Table 2 illustrates the complementarity of the nucleotide sequences of DKFZp566H0824 BINDING SITE, designated SEQ ID:1721, to the nucleotide sequence of VGAM130 RNA, herein designated VGAM RNA, also designated SEQ ID:465.

Another function of VGAM130 is therefore inhibition of DKFZp566H0824 (Accession NM_{—}017535). Accordingly, utilities of VGAM130 include diagnosis, prevention and treatment of diseases and clinical conditions associated with DKFZp566H0824. FLJ11726 (Accession NM_{—}024971) is another VGAM130 host target gene. FLJ11726 BINDING SITE is HOST TARGET binding site found in the 3′ untranslated region of mRNA encoded by FLJ11726, corresponding to a HOST TARGET binding site such as BINDING SITE I, BINDING SITE II or BINDING SITE III. Table 2 illustrates the complementarity of the nucleotide sequences of FLJ11726 BINDING SITE, designated SEQ ID:2119, to the nucleotide sequence of VGAM130 RNA, herein designated VGAM RNA, also designated SEQ ID:465.

Another function of VGAM130 is therefore inhibition of FLJ11726 (Accession NM_{—}024971). Accordingly, utilities of VGAM130 include diagnosis, prevention and treatment of diseases and clinical conditions associated with FLJ11726. FLJ12668 (Accession NM_{—}024997) is another VGAM130 host target gene. FLJ12668 BINDING SITE is HOST TARGET binding site found in the 3′ untranslated region of mRNA encoded by FLJ12668, corresponding to a HOST TARGET binding site such as BINDING SITE I, BINDING SITE II or BINDING SITE III. Table 2 illustrates the complementarity of the nucleotide sequences of FLJ12668 BINDING SITE, designated SEQ ID:2124, to the nucleotide sequence of VGAM130 RNA, herein designated VGAM RNA, also designated SEQ ID:465.

Another function of VGAM130 is therefore inhibition of FLJ12668 (Accession NM_{—}024997). Accordingly, utilities of VGAM130 include diagnosis, prevention and treatment of diseases and clinical conditions associated with FLJ12668. FLJ13385 (Accession NM_{—}024853) is another VGAM130 host target gene. FLJ13385 BINDING SITE is HOST TARGET binding site found in the 3′ untranslated region of mRNA encoded by FLJ13385, corresponding to a HOST TARGET binding site such as BINDING SITE I, BINDING SITE II or BINDING SITE III. Table 2 illustrates the complementarity of the nucleotide sequences of FLJ13385 BINDING SITE, designated SEQ ID:2098, to the nucleotide sequence of VGAM130 RNA, herein designated VGAM RNA, also designated SEQ ID:465.

Another function of VGAM130 is therefore inhibition of FLJ13385 (Accession NM_{—}024853). Accordingly, utilities of VGAM130 include diagnosis, prevention and treatment of diseases and clinical conditions associated with FLJ13385. FLJ14936 (Accession NM_{—}032284) is another VGAM130 host target gene. FLJ14936 BINDING SITE is HOST TARGET binding site found in the 3′ untranslated region of mRNA encoded by FLJ14936, corresponding to a HOST TARGET binding site such as BINDING SITE I, BINDING SITE II or BINDING SITE III. Table 2 illustrates the complementarity of the nucleotide sequences of FLJ14936 BINDING SITE, designated SEQ ID:2243, to the nucleotide sequence of VGAM130 RNA, herein designated VGAM RNA, also designated SEQ ID:465.

Another function of VGAM130 is therefore inhibition of FLJ14936 (Accession NM_{—}032284). Accordingly, utilities of VGAM130 include diagnosis, prevention and treatment of diseases and clinical conditions associated with FLJ14936. FLJ14957 (Accession NM_{—}032866) is another VGAM130 host target gene. FLJ14957 BINDING SITE is HOST TARGET binding site found in the 3′ untranslated region of mRNA encoded by FLJ14957, corresponding to a HOST TARGET binding site such as BINDING SITE I, BINDING SITE II or BINDING SITE III. Table 2 illustrates the complementarity of the nucleotide sequences of FLJ14957 BINDING SITE, designated SEQ ID:2291, to the nucleotide sequence of VGAM130 RNA, herein designated VGAM RNA, also designated SEQ ID:465.

Another function of VGAM130 is therefore inhibition of FLJ14957 (Accession NM_{—}032866). Accordingly, utilities of VGAM130 include diagnosis, prevention and treatment of diseases and clinical conditions associated with FLJ14957. HSU79275 (Accession XM_{—}038959) is another VGAM130 host target gene. HSU79275 BINDING SITE is HOST TARGET binding site found in the 3′ untranslated region of mRNA encoded by HSU79275, corresponding to a HOST TARGET binding site such as BINDING SITE I, BINDING SITE II or BINDING SITE III. Table 2 illustrates the complementarity of the nucleotide sequences of HSU79275 BINDING SITE, designated SEQ ID:2746, to the nucleotide sequence of VGAM130 RNA, herein designated VGAM RNA, also designated SEQ ID:465.

Another function of VGAM130 is therefore inhibition of HSU79275 (Accession XM_{—}038959). Accordingly, utilities of VGAM130 include diagnosis, prevention and treatment of diseases and clinical conditions associated with HSU79275. KALI (Accession NM_{—}052931) is another VGAM130 host target gene. KALI BINDING SITE is HOST TARGET binding site found in the 3′ untranslated region of mRNA encoded by KALI, corresponding to a HOST TARGET binding site such as BINDING SITE I, BINDING SITE II or BINDING SITE III. Table 2 illustrates the complementarity of the nucleotide sequences of KALI BINDING SITE, designated SEQ ID:2346, to the nucleotide sequence of VGAM130 RNA, herein designated VGAM RNA, also designated SEQ ID:465.

Another function of VGAM130 is therefore inhibition of KALI (Accession NM_{—}052931). Accordingly, utilities of VGAM130 include diagnosis, prevention and treatment of diseases and clinical conditions associated with KALI. KIAA0121 (Accession XM_{—}052386) is another VGAM130 host target gene. KIAA0121 BINDING SITE is HOST TARGET binding site found in the 3′ untranslated region of mRNA encoded by KIAA0121, corresponding to a HOST TARGET binding site such as BINDING SITE I, BINDING SITE II or BINDING SITE III. Table 2 illustrates the complementarity of the nucleotide sequences of KIAA0121 BINDING SITE, designated SEQ ID:2957, to the nucleotide sequence of VGAM130 RNA, herein designated VGAM RNA, also designated SEQ ID:465.

Another function of VGAM130 is therefore inhibition of KIAA0121 (Accession XM_{—}052386). Accordingly, utilities of VGAM130 include diagnosis, prevention and treatment of diseases and clinical conditions associated with KIAA0121. KIAA0254 (Accession NM_{—}014758) is another VGAM130 host target gene. KIAA0254 BINDING SITE is HOST TARGET binding site found in the 3′ untranslated region of mRNA encoded by KIAA0254, corresponding to a HOST TARGET binding site such as BINDING SITE I, BINDING SITE II or BINDING SITE III. Table 2 illustrates the complementarity of the nucleotide sequences of KIAA0254 BINDING SITE, designated SEQ ID:1540, to the nucleotide sequence of VGAM130 RNA, herein designated VGAM RNA, also designated SEQ ID:465.

Another function of VGAM130 is therefore inhibition of KIAA0254 (Accession NM_{—}014758). Accordingly, utilities of VGAM130 include diagnosis, prevention and treatment of diseases and clinical conditions associated with KIAA0254. KIAA0820 (Accession XM_{—}044463) is another VGAM130 host target gene. KIAA0820 BINDING SITE is HOST TARGET binding site found in the 3′ untranslated region of mRNA encoded by KIAA0820, corresponding to a HOST TARGET binding site such as BINDING SITE I, BINDING SITE II or BINDING SITE III. Table 2 illustrates the complementarity of the nucleotide sequences of KIAA0820 BINDING SITE, designated SEQ ID:2838, to the nucleotide sequence of VGAM130 RNA, herein designated VGAM RNA, also designated SEQ ID:465.

Another function of VGAM130 is therefore inhibition of KIAA0820 (Accession XM_{—}044463). Accordingly, utilities of VGAM130 include diagnosis, prevention and treatment of diseases and clinical conditions associated with KIAA0820. KIAA0863 (Accession XM_{—}170863) is another VGAM130 host target gene. KIAA0863 BINDING SITE is HOST TARGET binding site found in the 3′ untranslated region of mRNA encoded by KIAA0863, corresponding to a HOST TARGET binding site such as BINDING SITE I, BINDING SITE II or BINDING SITE III. Table 2 illustrates the complementarity of the nucleotide sequences of KIAA0863 BINDING SITE, designated SEQ ID:3669, to the nucleotide sequence of VGAM130 RNA, herein designated VGAM RNA, also designated SEQ ID:465.

Another function of VGAM130 is therefore inhibition of KIAA0863 (Accession XM_{—}170863). Accordingly, utilities of VGAM130 include diagnosis, prevention and treatment of diseases and clinical conditions associated with KIAA0863. KIAA1023 (Accession NM_{—}017604) is another VGAM130 host target gene. KIAA1023 BINDING SITE is HOST TARGET binding site found in the 3′ untranslated region of mRNA encoded by KIAA1023, corresponding to a HOST TARGET binding site such as BINDING SITE I, BINDING SITE II or BINDING SITE III. Table 2 illustrates the complementarity of the nucleotide sequences of KIAA1023 BINDING SITE, designated SEQ ID:1728, to the nucleotide sequence of VGAM130 RNA, herein designated VGAM RNA, also designated SEQ ID:465.

Another function of VGAM130 is therefore inhibition of KIAA1023 (Accession NM_{—}017604). Accordingly, utilities of VGAM130 include diagnosis, prevention and treatment of diseases and clinical conditions associated with KIAA1023. KIAA1161 (Accession XM_{—}088501) is another VGAM130 host target gene. KIAA1161 BINDING SITE is HOST TARGET binding site found in the 5′ untranslated region of mRNA encoded by KIAA1161, corresponding to a HOST TARGET binding site such as BINDING SITE I, BINDING SITE II or BINDING SITE III. Table 2 illustrates the complementarity of the nucleotide sequences of KIAA1161 BINDING SITE, designated SEQ ID:3213, to the nucleotide sequence of VGAM130 RNA, herein designated VGAM RNA, also designated SEQ ID:465.

Another function of VGAM130 is therefore inhibition of KIAA1161 (Accession XM_{—}088501). Accordingly, utilities of VGAM130 include diagnosis, prevention and treatment of diseases and clinical conditions associated with KIAA1161. KIAA1254 (Accession XM_{—}046132) is another VGAM130 host target gene. KIAA1254 BINDING SITE is HOST TARGET binding site found in the 3′ untranslated region of mRNA encoded by KIAA1254, corresponding to a HOST TARGET binding site such as BINDING SITE I, BINDING SITE II or BINDING SITE III. Table 2 illustrates the complementarity of the nucleotide sequences of KIAA1254 BINDING SITE, designated SEQ ID:2876, to the nucleotide sequence of VGAM130 RNA, herein designated VGAM RNA, also designated SEQ ID:465.

Another function of VGAM130 is therefore inhibition of KIAA1254 (Accession XM_{—}046132). Accordingly, utilities of VGAM130 include diagnosis, prevention and treatment of diseases and clinical conditions associated with KIAA1254. KIAA1805 (Accession XM_{—}086976) is another VGAM130 host target gene. KIAA1805 BINDING SITE is HOST TARGET binding site found in the 3′ untranslated region of mRNA encoded by KIAA1805, corresponding to a HOST TARGET binding site such as BINDING SITE I, BINDING SITE II or BINDING SITE III. Table 2 illustrates the complementarity of the nucleotide sequences of KIAA1805 BINDING SITE, designated SEQ ID:3148, to the nucleotide sequence of VGAM130 RNA, herein designated VGAM RNA, also designated SEQ ID:465.

Another function of VGAM130 is therefore inhibition of KIAA1805 (Accession XM_{—}086976). Accordingly, utilities of VGAM130 include diagnosis, prevention and treatment of diseases and clinical conditions associated with KIAA1805. KIAA1866 (Accession XM_{—}027658) is another VGAM130 host target gene. KIAA1866 BINDING SITE is HOST TARGET binding site found in the 3′ untranslated region of mRNA encoded by KIAA1866, corresponding to a HOST TARGET binding site such as BINDING SITE I, BINDING SITE II or BINDING SITE III. Table 2 illustrates the complementarity of the nucleotide sequences of KIAA1866 BINDING SITE, designated SEQ ID:2587, to the nucleotide sequence of VGAM130 RNA, herein designated VGAM RNA, also designated SEQ ID:465.

Another function of VGAM130 is therefore inhibition of KIAA1866 (Accession XM_{—}027658). Accordingly, utilities of VGAM130 include diagnosis, prevention and treatment of diseases and clinical conditions associated with KIAA1866. KIAA1958 (Accession XM_{—}088566) is another VGAM130 host target gene. KIAA1958 BINDING SITE is HOST TARGET binding site found in the 3′ untranslated region of mRNA encoded by KIAA1958, corresponding to a HOST TARGET binding site such as BINDING SITE I, BINDING SITE II or BINDING SITE III. Table 2 illustrates the complementarity of the nucleotide sequences of KIAA1958 BINDING SITE, designated SEQ ID:3218, to the nucleotide sequence of VGAM130 RNA, herein designated VGAM RNA, also designated SEQ ID:465.

Another function of VGAM130 is therefore inhibition of KIAA1958 (Accession XM_{—}088566). Accordingly, utilities of VGAM130 include diagnosis, prevention and treatment of diseases and clinical conditions associated with KIAA1958. LIM Domain Kinase 2 (LIMK2, Accession NM_{—}016733) is another VGAM130 host target gene. LIMK2 BINDING SITE is HOST TARGET binding site found in the 5′ untranslated region of mRNA encoded by LIMK2, corresponding to a HOST TARGET binding site such as BINDING SITE I, BINDING SITE II or BINDING SITE III. Table 2 illustrates the complementarity of the nucleotide sequences of LIMK2 BINDING SITE, designated SEQ ID:1709, to the nucleotide sequence of VGAM130 RNA, herein designated VGAM RNA, also designated SEQ ID:465.

Another function of VGAM130 is therefore inhibition of LIM Domain Kinase 2 (LIMK2, Accession NM_{—}016733). Accordingly, utilities of VGAM130 include diagnosis, prevention and treatment of diseases and clinical conditions associated with LIMK2. MGC12921 (Accession XM_{—}033362) is another VGAM130 host target gene. MGC12921 BINDING SITE is HOST TARGET binding site found in the 5′ untranslated region of mRNA encoded by MGC12921, corresponding to a HOST TARGET binding site such as BINDING SITE I, BINDING SITE II or BINDING SITE III. Table 2 illustrates the complementarity of the nucleotide sequences of MGC12921 BINDING SITE, designated SEQ ID:2666, to the nucleotide sequence of VGAM130 RNA, herein designated VGAM RNA, also designated SEQ ID:465.

Another function of VGAM130 is therefore inhibition of MGC12921 (Accession XM_{—}033362). Accordingly, utilities of VGAM130 include diagnosis, prevention and treatment of diseases and clinical conditions associated with MGC12921. MGC5149 (Accession XM_{—}051200) is another VGAM130 host target gene. MGC5149 BINDING SITE is HOST TARGET binding site found in the 3′ untranslated region of mRNA encoded by MGC5149, corresponding to a HOST TARGET binding site such as BINDING SITE I, BINDING SITE II or BINDING SITE III. Table 2 illustrates the complementarity of the nucleotide sequences of MGC5149 BINDING SITE, designated SEQ ID:2939, to the nucleotide sequence of VGAM130 RNA, herein designated VGAM RNA, also designated SEQ ID:465.

Another function of VGAM130 is therefore inhibition of MGC5149 (Accession XM_{—}051200). Accordingly, utilities of VGAM130 include diagnosis, prevention and treatment of diseases and clinical conditions associated with MGC5149. MOST2 (Accession NM_{—}020250) is another VGAM130 host target gene. MOST2 BINDING SITE is HOST TARGET binding site found in the 5′ untranslated region of mRNA encoded by MOST2, corresponding to a HOST TARGET binding site such as BINDING SITE I, BINDING SITE II or BINDING SITE III. Table 2 illustrates the complementarity of the nucleotide sequences of MOST2 BINDING SITE, designated SEQ ID:1909, to the nucleotide sequence of VGAM130 RNA, herein designated VGAM RNA, also designated SEQ ID:465.

Another function of VGAM130 is therefore inhibition of MOST2 (Accession NM_{—}020250). Accordingly, utilities of VGAM130 include diagnosis, prevention and treatment of diseases and clinical conditions associated with MOST2. Mitochondrial Ribosomal Protein L9 (MRPL9, Accession NM_{—}031420) is another VGAM130 host target gene. MRPL9 BINDING SITE is HOST TARGET binding site found in the 3′ untranslated region of mRNA encoded by MRPL9, corresponding to a HOST TARGET binding site such as BINDING SITE I, BINDING SITE II or BINDING SITE III. Table 2 illustrates the complementarity of the nucleotide sequences of MRPL9 BINDING SITE, designated SEQ ID:2195, to the nucleotide sequence of VGAM130 RNA, herein designated VGAM RNA, also designated SEQ ID:465.

Another function of VGAM130 is therefore inhibition of Mitochondrial Ribosomal Protein L9 (MRPL9, Accession NM_{—}031420). Accordingly, utilities of VGAM130 include diagnosis, prevention and treatment of diseases and clinical conditions associated with MRPL9. Nuclear Cap Binding Protein Subunit 2, 20 kDa (NCBP2, Accession NM_{—}007362) is another VGAM130 host target gene. NCBP2 BINDING SITE is HOST TARGET binding site found in the 3′ untranslated region of mRNA encoded by NCBP2, corresponding to a HOST TARGET binding site such as BINDING SITE I, BINDING SITE II or BINDING SITE III. Table 2 illustrates the complementarity of the nucleotide sequences of NCBP2 BINDING SITE, designated SEQ ID:1397, to the nucleotide sequence of VGAM130 RNA, herein designated VGAM RNA, also designated SEQ ID:465.

Another function of VGAM130 is therefore inhibition of Nuclear Cap Binding Protein Subunit 2, 20 kDa (NCBP2, Accession NM_{—}007362). Accordingly, utilities of VGAM130 include diagnosis, prevention and treatment of diseases and clinical conditions associated with NCBP2. Phosphate Cytidylyltransferase 1, Choline, Beta Isoform (PCYT1B, Accession NM_{—}004845) is another VGAM130 host target gene. PCYT1B BINDING SITE is HOST TARGET binding site found in the 3′ untranslated region of mRNA encoded by PCYT1B, corresponding to a HOST TARGET binding site such as BINDING SITE I, BINDING SITE II or BINDING SITE III. Table 2 illustrates the complementarity of the nucleotide sequences of PCYT1B BINDING SITE, designated SEQ ID:1160, to the nucleotide sequence of VGAM130 RNA, herein designated VGAM RNA, also designated SEQ ID:465.

Another function of VGAM130 is therefore inhibition of Phosphate Cytidylyltransferase 1, Choline, Beta Isoform (PCYT1B, Accession NM_{—}004845). Accordingly, utilities of VGAM130 include diagnosis, prevention and treatment of diseases and clinical conditions associated with PCYT1B. PIP3-E (Accession XM_{—}039749) is another VGAM130 host target gene. PIP3-E BINDING SITE is HOST TARGET binding site found in the 3′ untranslated region of mRNA encoded by PIP3-E, corresponding to a HOST TARGET binding site such as BINDING SITE I, BINDING SITE II or BINDING SITE III. Table 2 illustrates the complementarity of the nucleotide sequences of PIP3-E BINDING SITE, designated SEQ ID:2764, to the nucleotide sequence of VGAM130 RNA, herein designated VGAM RNA, also designated SEQ ID:465.

Another function of VGAM130 is therefore inhibition of PIP3-E (Accession XM_{—}039749). Accordingly, utilities of VGAM130 include diagnosis, prevention and treatment of diseases and clinical conditions associated with PIP3-E. Protein Phosphatase 1, Regulatory (inhibitor) Subunit 15B (PPP1R15B, Accession NM_{—}032833) is another VGAM130 host target gene. PPP1R15B BINDING SITE is HOST TARGET binding site found in the 5′ untranslated region of mRNA encoded by PPP1R15B, corresponding to a HOST TARGET binding site such as BINDING SITE I, BINDING SITE II or BINDING SITE III. Table 2 illustrates the complementarity of the nucleotide sequences of PPP1R15B BINDING SITE, designated SEQ ID:2288, to the nucleotide sequence of VGAM130 RNA, herein designated VGAM RNA, also designated SEQ ID:465.

Another function of VGAM130 is therefore inhibition of Protein Phosphatase 1, Regulatory (inhibitor) Subunit 15B (PPP1R15B, Accession NM_{—}032833). Accordingly, utilities of VGAM130 include diagnosis, prevention and treatment of diseases and clinical conditions associated with PPP1R15B. Tuftelin Interacting Protein 11 (TFIP11, Accession NM_{—}012143) is another VGAM130 host target gene. TFIP11 BINDING SITE is HOST TARGET binding site found in the 3′ untranslated region of mRNA encoded by TFIP11, corresponding to a HOST TARGET binding site such as BINDING SITE I, BINDING SITE II or BINDING SITE III. Table 2 illustrates the complementarity of the nucleotide sequences of TFIP11 BINDING SITE, designated SEQ ID:1410, to the nucleotide sequence of VGAM130 RNA, herein designated VGAM RNA, also designated SEQ ID:465.

Another function of VGAM130 is therefore inhibition of Tuftelin Interacting Protein 11 (TFIP11, Accession NM_{—}012143). Accordingly, utilities of VGAM130 include diagnosis, prevention and treatment of diseases and clinical conditions associated with TFIP11. LOC132617 (Accession XM_{—}067939) is another VGAM130 host target gene. LOC132617 BINDING SITE is HOST TARGET binding site found in the 3′ untranslated region of mRNA encoded by LOC132617, corresponding to a HOST TARGET binding site such as BINDING SITE I, BINDING SITE II or BINDING SITE III. Table 2 illustrates the complementarity of the nucleotide sequences of LOC132617 BINDING SITE, designated SEQ ID:3039, to the nucleotide sequence of VGAM130 RNA, herein designated VGAM RNA, also designated SEQ ID:465.

Another function of VGAM130 is therefore inhibition of LOC132617 (Accession XM_{—}067939). Accordingly, utilities of VGAM130 include diagnosis, prevention and treatment of diseases and clinical conditions associated with LOC132617. LOC147660 (Accession XM_{—}085825) is another VGAM130 host target gene. LOC147660 BINDING SITE is HOST TARGET binding site found in the 3′ untranslated region of mRNA encoded by LOC147660, corresponding to a HOST TARGET binding site such as BINDING SITE I, BINDING SITE II or BINDING SITE III. Table 2 illustrates the complementarity of the nucleotide sequences of LOC147660 BINDING SITE, designated SEQ ID:3111, to the nucleotide sequence of VGAM130 RNA, herein designated VGAM RNA, also designated SEQ ID:465.

Another function of VGAM130 is therefore inhibition of LOC147660 (Accession XM_{—}085825). Accordingly, utilities of VGAM130 include diagnosis, prevention and treatment of diseases and clinical conditions associated with LOC147660. LOC149302 (Accession XM_{—}086489) is another VGAM130 host target gene. LOC149302 BINDING SITE is HOST TARGET binding site found in the 5′ untranslated region of mRNA encoded by LOC149302, corresponding to a HOST TARGET binding site such as BINDING SITE I, BINDING SITE II or BINDING SITE III. Table 2 illustrates the complementarity of the nucleotide sequences of LOC149302 BINDING SITE, designated SEQ ID:3129, to the nucleotide sequence of VGAM130 RNA, herein designated VGAM RNA, also designated SEQ ID:465.

Another function of VGAM130 is therefore inhibition of LOC149302 (Accession XM_{—}086489). Accordingly, utilities of VGAM130 include diagnosis, prevention and treatment of diseases and clinical conditions associated with LOC149302. LOC151720 (Accession XM_{—}087279) is another VGAM130 host target gene. LOC151720 BINDING SITE is HOST TARGET binding site found in the 5′ untranslated region of mRNA encoded by LOC151720, corresponding to a HOST TARGET binding site such as BINDING SITE I, BINDING SITE II or BINDING SITE III. Table 2 illustrates the complementarity of the nucleotide sequences of LOC151720 BINDING SITE, designated SEQ ID:3160, to the nucleotide sequence of VGAM130 RNA, herein designated VGAM RNA, also designated SEQ ID:465.

Another function of VGAM130 is therefore inhibition of LOC151720 (Accession XM_{—}087279). Accordingly, utilities of VGAM130 include diagnosis, prevention and treatment of diseases and clinical conditions associated with LOC151720. LOC152215 (Accession XM_{—}087407) is another VGAM130 host target gene. LOC152215 BINDING SITE is HOST TARGET binding site found in the 3′ untranslated region of mRNA encoded by LOC152215, corresponding to a HOST TARGET binding site such as BINDING SITE I, BINDING SITE II or BINDING SITE III. Table 2 illustrates the complementarity of the nucleotide sequences of LOC152215 BINDING SITE, designated SEQ ID:3167, to the nucleotide sequence of VGAM130 RNA, herein designated VGAM RNA, also designated SEQ ID:465.

Another function of VGAM130 is therefore inhibition of LOC152215 (Accession XM_{—}087407). Accordingly, utilities of VGAM130 include diagnosis, prevention and treatment of diseases and clinical conditions associated with LOC152215. LOC152426 (Accession XM_{—}098225) is another VGAM130 host target gene. LOC152426 BINDING SITE is HOST TARGET binding site found in the 3′ untranslated region of mRNA encoded by LOC152426, corresponding to a HOST TARGET binding site such as BINDING SITE I, BINDING SITE II or BINDING SITE III. Table 2 illustrates the complementarity of the nucleotide sequences of LOC152426 BINDING SITE, designated SEQ ID:3359, to the nucleotide sequence of VGAM130 RNA, herein designated VGAM RNA, also designated SEQ ID:465.

Another function of VGAM130 is therefore inhibition of LOC152426 (Accession XM_{—}098225). Accordingly, utilities of VGAM130 include diagnosis, prevention and treatment of diseases and clinical conditions associated with LOC152426. LOC155179 (Accession XM_{—}088169) is another VGAM130 host target gene. LOC155179 BINDING SITE is HOST TARGET binding site found in the 3′ untranslated region of mRNA encoded by LOC155179, corresponding to a HOST TARGET binding site such as BINDING SITE I, BINDING SITE II or BINDING SITE III. Table 2 illustrates the complementarity of the nucleotide sequences of LOC155179 BINDING SITE, designated SEQ ID:3199, to the nucleotide sequence of VGAM130 RNA, herein designated VGAM RNA, also designated SEQ ID:465.

Another function of VGAM130 is therefore inhibition of LOC155179 (Accession XM_{—}088169). Accordingly, utilities of VGAM130 include diagnosis, prevention and treatment of diseases and clinical conditions associated with LOC155179. LOC157562 (Accession XM_{—}098779) is another VGAM130 host target gene. LOC157562 BINDING SITE is HOST TARGET binding site found in the 5′ untranslated region of mRNA encoded by LOC157562, corresponding to a HOST TARGET binding site such as BINDING SITE I, BINDING SITE II or BINDING SITE III. Table 2 illustrates the complementarity of the nucleotide sequences of LOC157562 BINDING SITE, designated SEQ ID:3381, to the nucleotide sequence of VGAM130 RNA, herein designated VGAM RNA, also designated SEQ ID:465.

Another function of VGAM130 is therefore inhibition of LOC157562 (Accession XM_{—}098779). Accordingly, utilities of VGAM130 include diagnosis, prevention and treatment of diseases and clinical conditions associated with LOC157562. LOC163115 (Accession XM_{—}092010) is another VGAM130 host target gene. LOC163115 BINDING SITE is HOST TARGET binding site found in the 3′ untranslated region of mRNA encoded by LOC163115, corresponding to a HOST TARGET binding site such as BINDING SITE I, BINDING SITE II or BINDING SITE III. Table 2 illustrates the complementarity of the nucleotide sequences of LOC163115 BINDING SITE, designated SEQ ID:3244, to the nucleotide sequence of VGAM130 RNA, herein designated VGAM RNA, also designated SEQ ID:465.

Another function of VGAM130 is therefore inhibition of LOC163115 (Accession XM_{—}092010). Accordingly, utilities of VGAM130 include diagnosis, prevention and treatment of diseases and clinical conditions associated with LOC163115. LOC169026 (Accession XM_{—}095471) is another VGAM130 host target gene. LOC169026 BINDING SITE is HOST TARGET binding site found in the 3′ untranslated region of mRNA encoded by LOC169026, corresponding to a HOST TARGET binding site such as BINDING SITE I, BINDING SITE II or BINDING SITE III. Table 2 illustrates the complementarity of the nucleotide sequences of LOC169026 BINDING SITE, designated SEQ ID:3260, to the nucleotide sequence of VGAM130 RNA, herein designated VGAM RNA, also designated SEQ ID:465.

Another function of VGAM130 is therefore inhibition of LOC169026 (Accession XM_{—}095471). Accordingly, utilities of VGAM130 include diagnosis, prevention and treatment of diseases and clinical conditions associated with LOC169026. LOC170106 (Accession XM_{—}093106) is another VGAM130 host target gene. LOC170106 BINDING SITE is HOST TARGET binding site found in the 5′ untranslated region of mRNA encoded by LOC170106, corresponding to a HOST TARGET binding site such as BINDING SITE I, BINDING SITE II or BINDING SITE III. Table 2 illustrates the complementarity of the nucleotide sequences of LOC170106 BINDING SITE, designated SEQ ID:3255, to the nucleotide sequence of VGAM130 RNA, herein designated VGAM RNA, also designated SEQ ID:465.

Another function of VGAM130 is therefore inhibition of LOC170106 (Accession XM_{—}093106). Accordingly, utilities of VGAM130 include diagnosis, prevention and treatment of diseases and clinical conditions associated with LOC170106. LOC170425 (Accession XM_{—}084330) is another VGAM130 host target gene. LOC170425 BINDING SITE is HOST TARGET binding site found in the 5′ untranslated region of mRNA encoded by LOC170425, corresponding to a HOST TARGET binding site such as BINDING SITE I, BINDING SITE II or BINDING SITE III. Table 2 illustrates the complementarity of the nucleotide sequences of LOC170425 BINDING SITE, designated SEQ ID:3048, to the nucleotide sequence of VGAM130 RNA, herein designated VGAM RNA, also designated SEQ ID:465.

Another function of VGAM130 is therefore inhibition of LOC170425 (Accession XM_{—}084330). Accordingly, utilities of VGAM130 include diagnosis, prevention and treatment of diseases and clinical conditions associated with LOC170425. LOC197342 (Accession XM_{—}113869) is another VGAM130 host target gene. LOC197342 BINDING SITE is HOST TARGET binding site found in the 3′ untranslated region of mRNA encoded by LOC197342, corresponding to a HOST TARGET binding site such as BINDING SITE I, BINDING SITE II or BINDING SITE III. Table 2 illustrates the complementarity of the nucleotide sequences of LOC197342 BINDING SITE, designated SEQ ID:3423, to the nucleotide sequence of VGAM130 RNA, herein designated VGAM RNA, also designated SEQ ID:465.

Another function of VGAM130 is therefore inhibition of LOC197342 (Accession XM_{—}113869). Accordingly, utilities of VGAM130 include diagnosis, prevention and treatment of diseases and clinical conditions associated with LOC197342. LOC200488 (Accession XM_{—}117240) is another VGAM130 host target gene. LOC200488 BINDING SITE is HOST TARGET binding site found in the 5′ untranslated region of mRNA encoded by LOC200488, corresponding to a HOST TARGET binding site such as BINDING SITE I, BINDING SITE II or BINDING SITE III. Table 2 illustrates the complementarity of the nucleotide sequences of LOC200488 BINDING SITE, designated SEQ ID:3477, to the nucleotide sequence of VGAM130 RNA, herein designated VGAM RNA, also designated SEQ ID:465.

Another function of VGAM130 is therefore inhibition of LOC200488 (Accession XM_{—}117240). Accordingly, utilities of VGAM130 include diagnosis, prevention and treatment of diseases and clinical conditions associated with LOC200488. LOC219667 (Accession XM_{—}166098) is another VGAM130 host target gene. LOC219667 BINDING SITE is HOST TARGET binding site found in the 3′ untranslated region of mRNA encoded by LOC219667, corresponding to a HOST TARGET binding site such as BINDING SITE I, BINDING SITE II or BINDING SITE III. Table 2 illustrates the complementarity of the nucleotide sequences of LOC219667 BINDING SITE, designated SEQ ID:3517, to the nucleotide sequence of VGAM130 RNA, herein designated VGAM RNA, also designated SEQ ID:465.

Another function of VGAM130 is therefore inhibition of LOC219667 (Accession XM_{—}166098). Accordingly, utilities of VGAM130 include diagnosis, prevention and treatment of diseases and clinical conditions associated with LOC219667. LOC220980 (Accession XM_{—}167629) is another VGAM130 host target gene. LOC220980 BINDING SITE is HOST TARGET binding site found in the 5′ untranslated region of mRNA encoded by LOC220980, corresponding to a HOST TARGET binding site such as BINDING SITE I, BINDING SITE II or BINDING SITE III. Table 2 illustrates the complementarity of the nucleotide sequences of LOC220980 BINDING SITE, designated SEQ ID:3592, to the nucleotide sequence of VGAM130 RNA, herein designated VGAM RNA, also designated SEQ ID:465.

Another function of VGAM130 is therefore inhibition of LOC220980 (Accession XM_{—}167629). Accordingly, utilities of VGAM130 include diagnosis, prevention and treatment of diseases and clinical conditions associated with LOC220980. LOC253461 (Accession XM_{—}172341) is another VGAM130 host target gene. LOC253461 BINDING SITE is HOST TARGET binding site found in the 3′ untranslated region of mRNA encoded by LOC253461, corresponding to a HOST TARGET binding site such as BINDING SITE I, BINDING SITE II or BINDING SITE III. Table 2 illustrates the complementarity of the nucleotide sequences of LOC253461 BINDING SITE, designated SEQ ID:3700, to the nucleotide sequence of VGAM130 RNA, herein designated VGAM RNA, also designated SEQ ID:465.

Another function of VGAM130 is therefore inhibition of LOC253461 (Accession XM_{—}172341). Accordingly, utilities of VGAM130 include diagnosis, prevention and treatment of diseases and clinical conditions associated with LOC253461. LOC253502 (Accession XM_{—}170561) is another VGAM130 host target gene. LOC253502 BINDING SITE is HOST TARGET binding site found in the 3′ untranslated region of mRNA encoded by LOC253502, corresponding to a HOST TARGET binding site such as BINDING SITE I, BINDING SITE II or BINDING SITE III. Table 2 illustrates the complementarity of the nucleotide sequences of LOC253502 BINDING SITE, designated SEQ ID:3660, to the nucleotide sequence of VGAM130 RNA, herein designated VGAM RNA, also designated SEQ ID:465.

Another function of VGAM130 is therefore inhibition of LOC253502 (Accession XM_{—}170561). Accordingly, utilities of VGAM130 include diagnosis, prevention and treatment of diseases and clinical conditions associated with LOC253502. LOC254211 (Accession XM_{—}172845) is another VGAM130 host target gene. LOC254211 BINDING SITE is HOST TARGET binding site found in the 5′ untranslated region of mRNA encoded by LOC254211, corresponding to a HOST TARGET binding site such as BINDING SITE I, BINDING SITE II or BINDING SITE III. Table 2 illustrates the complementarity of the nucleotide sequences of LOC254211 BINDING SITE, designated SEQ ID:3707, to the nucleotide sequence of VGAM130 RNA, herein designated VGAM RNA, also designated SEQ ID:465.

Another function of VGAM130 is therefore inhibition of LOC254211 (Accession XM_{—}172845). Accordingly, utilities of VGAM130 include diagnosis, prevention and treatment of diseases and clinical conditions associated with LOC254211. LOC90785 (Accession XM_{—}034110) is another VGAM130 host target gene. LOC90785 BINDING SITE is HOST TARGET binding site found in the 3′ untranslated region of mRNA encoded by LOC90785, corresponding to a HOST TARGET binding site such as BINDING SITE I, BINDING SITE II or BINDING SITE III. Table 2 illustrates the complementarity of the nucleotide sequences of LOC90785 BINDING SITE, designated SEQ ID:2677, to the nucleotide sequence of VGAM130 RNA, herein designated VGAM RNA, also designated SEQ ID:465.

Another function of VGAM130 is therefore inhibition of LOC90785 (Accession XM_{—}034110). Accordingly, utilities of VGAM130 include diagnosis, prevention and treatment of diseases and clinical conditions associated with LOC90785. LOC91050 (Accession XM_{—}035703) is another VGAM130 host target gene. LOC91050 BINDING SITE is HOST TARGET binding site found in the 3′ untranslated region of mRNA encoded by LOC91050, corresponding to a HOST TARGET binding site such as BINDING SITE I, BINDING SITE II or BINDING SITE III. Table 2 illustrates the complementarity of the nucleotide sequences of LOC91050 BINDING SITE, designated SEQ ID:2701, to the nucleotide sequence of VGAM130 RNA, herein designated VGAM RNA, also designated SEQ ID:465.

Another function of VGAM130 is therefore inhibition of LOC91050 (Accession XM_{—}035703). Accordingly, utilities of VGAM130 include diagnosis, prevention and treatment of diseases and clinical conditions associated with LOC91050. LOC91380 (Accession XM_{—}038134) is another VGAM130 host target gene. LOC91380 BINDING SITE is HOST TARGET binding site found in the 3′ untranslated region of mRNA encoded by LOC91380, corresponding to a HOST TARGET binding site such as BINDING SITE I, BINDING SITE II or BINDING SITE III. Table 2 illustrates the complementarity of the nucleotide sequences of LOC91380 BINDING SITE, designated SEQ ID:2733, to the nucleotide sequence of VGAM130 RNA, herein designated VGAM RNA, also designated SEQ ID:465.

Another function of VGAM130 is therefore inhibition of LOC91380 (Accession XM_{—}038134). Accordingly, utilities of VGAM130 include diagnosis, prevention and treatment of diseases and clinical conditions associated with LOC91380. FIG. 1 further provides a conceptual description of a novel bioinformatically detected viral gene of the present invention, referred to here as Viral Genomic Address Messenger 131 (VGAM131) viral gene, which modulates expression of respective host target genes thereof, the function and utility of which host target genes is known in the art.

VGAM131 is a novel bioinformatically detected regulatory, non protein coding, viral micro RNA (miRNA) gene. The method by which VGAM131 was detected is described hereinabove with reference to FIGS. 1-8.

VGAM131 gene, herein designated VGAM GENE, is a viral gene contained in the genome of Vaccinia Virus. VGAM131 host target gene, herein designated VGAM HOST TARGET GENE, is a human gene contained in the human genome.

VGAM131 gene encodes a VGAM131 precursor RNA, herein designated VGAM PRECURSOR RNA. Similar to other miRNA genes, and unlike most ordinary genes, VGAM131 precursor RNA does not encode a protein. A nucleotide sequence identical or highly similar to the nucleotide sequence of VGAM131 precursor RNA is designated SEQ ID:117, and is provided hereinbelow with reference to the sequence listing part. Nucleotide sequence SEQ ID:117 is located at position 60496 relative to the genome of Vaccinia Virus.

VGAM131 precursor RNA folds onto itself, forming VGAM131 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM131 folded precursor RNA into VGAM131 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM131 RNA is designated SEQ ID:466, and is provided hereinbelow with reference to the sequence listing part.

VGAM131 host target gene, herein designated VGAM HOST TARGET GENE, encodes a corresponding messenger RNA, VGAM131 host target RNA, herein designated VGAM HOST TARGET RNA. VGAM131 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM131 RNA, herein designated VGAM RNA, binds complementarily to one or more host target binding sites located in untranslated regions of VGAM131 host target RNA, herein designated VGAM HOST TARGET RNA. This complementary binding is due to the fact that the nucleotide sequence of VGAM131 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM131 RNA, herein designated VGAM RNA, may have a different number of host target binding sites in untranslated regions of a VGAM131 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM131 RNA, herein designated VGAM RNA, to host target binding sites on VGAM131 host target RNA, herein designated VGAM HOST TARGET RNA, such as BINDING SITE I, BINDING SITE II and BINDING SITE III, inhibits translation of VGAM131 host target RNA into VGAM131 host target protein, herein designated VGAM HOST TARGET PROTEIN. VGAM host target protein is therefore outlined by a broken line.

It is appreciated that VGAM131 host target gene, herein designated VGAM HOST TARGET GENE, in fact represents a plurality of VGAM131 host target genes. The mRNA of each one of this plurality of VGAM131 host target genes comprises one or more host target binding sites, each having a nucleotide sequence which is at least partly complementary to VGAM131 RNA, herein designated VGAM RNA, and which when bound by VGAM131 RNA causes inhibition of translation of respective one or more VGAM131 host target proteins.

It is further appreciated by one skilled in the art that the mode of translational inhibition illustrated by FIG. 1 with specific reference to translational inhibition exerted by VGAM131 gene, herein designated VGAM GENE, on one or more VGAM131 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM131 is inhibition of expression of host target genes, as part of a novel viral mechanism of attacking a host. Accordingly, utilities of VGAM131 include diagnosis, prevention and treatment of viral infection by Vaccinia Virus. Specific functions, and accordingly utilities, of VGAM131 correlate with, and may be deduced from, the identity of the host target genes which VGAM131 binds and inhibits, and the function of these host target genes, as elaborated hereinbelow.

Nucleotide sequences of the VGAM131 precursor RNA, herein designated VGAM PRECURSOR RNA, and of the ‘diced’ VGAM131 RNA, herein designated VGAM RNA, and a schematic representation of the secondary folding of VGAM131 folded precursor RNA, herein designated VGAM FOLDED PRECURSOR RNA, of VGAM131 are further described hereinbelow with reference to Table 1.

Nucleotide sequences of host target binding sites, such as BINDING SITE-I, BINDING SITE-II and BINDING SITE-III of FIG. 1, found on VGAM131 host target RNA, and schematic representation of the complementarity of each of these host target binding sites to VGAM131 RNA, herein designated VGAM RNA, are described hereinbelow with reference to Table 2.

As mentioned hereinabove with reference to FIG. 1, a function of VGAM131 gene, herein designated VGAM is inhibition of expression of VGAM131 target genes. It is appreciated that specific functions, and accordingly utilities, of VGAM131 correlate with, and may be deduced from, the identity of the target genes which VGAM131 binds and inhibits, and the function of these target genes, as elaborated hereinbelow.

Eukaryotic Translation Initiation Factor 2C, 1 (EIF2C1, Accession NM_{—}012199) is a VGAM131 host target gene. EIF2C1 BINDING SITE is HOST TARGET binding site found in the 3′ untranslated region of mRNA encoded by EIF2C1, corresponding to a HOST TARGET binding site such as BINDING SITE I, BINDING SITE II or BINDING SITE III. Table 2 illustrates the complementarity of the nucleotide sequences of EIF2C1 BINDING SITE, designated SEQ ID:1414, to the nucleotide sequence of VGAM131 RNA, herein designated VGAM RNA, also designated SEQ ID:466.

A function of VGAM131 is therefore inhibition of Eukaryotic Translation Initiation Factor 2C, 1 (EIF2C1, Accession NM_{—}012199), a gene which plays an important role in the eukaryotic peptide chain initiation process. Accordingly, utilities of VGAM131 include diagnosis, prevention and treatment of diseases and clinical conditions associated with EIF2C1. The function of EIF2C1 has been established by previous studies. Koesters et al. (1999) isolated a EIF2C1 cDNA from a human fetal kidney cDNA library. To obtain genomic sequence information, they isolated a P1 genomic clone containing the EIF2C1 locus. The human EIF2C1 gene encodes a protein of 857 amino acids. The 2,571-bp open reading frame is flanked by 238 bp of 5-prime sequence and an extremely large 3-prime untranslated region with multiple short repeated segments composed of mono-, tri-, or quatronucleotides interspersed throughout. Northern blot analysis demonstrated that the human EIF2C1 gene is ubiquitously expressed at low to medium levels. Differential polyadenylation and splicing resulted in a complex transcriptional pattern. Martinez et al. (2002) demonstrated that a single-stranded small interfering RNA (siRNA) resides in the human RNA-induced silencing complex (RISC) together with the EIF2C1 and/or EIF2C2 (OMIM Ref. No. 606229) proteins. RISC could be rapidly formed in HeLa cell cytoplasmic extract supplemented with 21-nucleotide siRNA duplexes, but also by adding single-stranded antisense RNAs, which range in size between 19 and 29 nucleotides.

Full details of the abovementioned studies are described in the following publications, the disclosure of which are hereby incorporated by reference:
Koesters, R.; Adams, V.; Betts, D.; Moos, R.; Schmid, M.; Siermann, A.; Hassam, S.; Weitz, S.; Lichter, P.; Heitz, P. U.; von Knebel Doeberitz, M.; Briner, J.: Human eukaryotic initiation factor EIF2C1 gene: cDNA sequence, genomic organization, localization to chromosomal bands 1q34-p35, and expression. Genomics 61: 210-218, 1999.; and
Martinez, J.; Patkaniowska, A.; Urlaub, H.; Luhrmann, R.; Tuschi, T.: Single-stranded antisense siRNAs guide target RNA cleavage in RNAi. Cell 110: 563-574, 2002.

Further studies establishing the function and utilities of EIF2C1 are found in John Hopkins OMIM database record ID 606228, and in sited publications numbered 1530-1531 listed in the bibliography section hereinbelow, which are also hereby incorporated by reference. Proto-cadherin Alpha 9 (PCDHA9, Accession NM_{—}014005) is another VGAM131 host target gene. PCDHA9 BINDING SITE is HOST TARGET binding site found in the 3′ untranslated region of mRNA encoded by PCDHA9, corresponding to a HOST TARGET binding site such as BINDING SITE I, BINDING SITE II or BINDING SITE III. Table 2 illustrates the complementarity of the nucleotide sequences of PCDHA9 BINDING SITE, designated SEQ ID:1461, to the nucleotide sequence of VGAM131 RNA, herein designated VGAM RNA, also designated SEQ ID:466.

Another function of VGAM131 is therefore inhibition of Proto-cadherin Alpha 9 (PCDHA9, Accession NM_{—}014005), a gene which is a calcium-dependent cell-adhesion protein. Accordingly, utilities of VGAM131 include diagnosis, prevention and treatment of diseases and clinical conditions associated with PCDHA9. The function of PCDHA9 has been established by previous studies. Cadherins are calcium-dependent cell-cell adhesion molecules that mediate neural cell-cell interactions. Proto-cadherins constitute a sub family of nonclassic cadherins. PCDHA9 is a member of the alpha cluster of protocadherin genes on 5q31. By screening a brain cDNA library for sequences with the potential to encode large proteins, Nagase et al. (1997) identified a cDNA encoding PCDHA9, which they termed KIAA0345. The deduced protein has 842 amino acids. RT-PCR analysis detected strongest expression of KIAA0345 in kidney and testis, followed by brain, lung, pancreas, and ovary.

Full details of the abovementioned studies are described in the following publications, the disclosure of which are hereby incorporated by reference:
Nagase, T.; Ishikawa, I.; Nakajima, D.; Ohira, M.; Seki, N.; Miyajima, N.; Tanaka, A.; Kotani, H.; Nomura, N.; O'Hara, O.: Prediction of the coding sequences of unidentified human genes. VII. The complete sequences of 100 new cDNA clones from brain which can code for large proteins in vitro. DNA Res. 4: 141-150, 1997.; and
Wu, Q.; Zhang, T.; Cheng, J.-F.; Kim, Y.; Grimwood, J.; Schmutz, J.; Dickson, M.; Noonan, J. P.; Zhang, M. Q.; Myers, R. M.; Maniatis, T.: Comparative DNA sequence analysis of mouse and.

Further studies establishing the function and utilities of PCDHA9 are found in John Hopkins OMIM database record ID 606315, and in sited publications numbered 165 and 2153 listed in the bibliography section hereinbelow, which are also hereby incorporated by reference. KIAA0923 (Accession NM_{—}014021) is another VGAM131 host target gene. KIAA0923 BINDING SITE is HOST TARGET binding site found in the 3′ untranslated region of mRNA encoded by KIAA0923, corresponding to a HOST TARGET binding site such as BINDING SITE I, BINDING SITE II or BINDING SITE III. Table 2 illustrates the complementarity of the nucleotide sequences of KIAA0923 BINDING SITE, designated SEQ ID: 1463, to the nucleotide sequence of VGAM131 RNA, herein designated VGAM RNA, also designated SEQ ID:466.

Another function of VGAM131 is therefore inhibition of KIAA0923 (Accession NM_{—}014021). Accordingly, utilities of VGAM131 include diagnosis, prevention and treatment of diseases and clinical conditions associated with KIAA0923. UBF-fl (Accession NM_{—}032828) is another VGAM131 host target gene. UBF-fl BINDING SITE is HOST TARGET binding site found in the 3′ untranslated region of mRNA encoded by UBF-fl, corresponding to a HOST TARGET binding site such as BINDING SITE I, BINDING SITE II or BINDING SITE III. Table 2 illustrates the complementarity of the nucleotide sequences of UBF-fl BINDING SITE, designated SEQ ID:2287, to the nucleotide sequence of VGAM131 RNA, herein designated VGAM RNA, also designated SEQ ID:466.

Another function of VGAM131 is therefore inhibition of UBF-fl (Accession NM_{—}032828). Accordingly, utilities of VGAM131 include diagnosis, prevention and treatment of diseases and clinical conditions associated with UBF-fl. LOC119548 (Accession XM_{—}058404) is another VGAM131 host target gene. LOC119548 BINDING SITE is HOST TARGET binding site found in the 3′ untranslated region of mRNA encoded by LOC119548, corresponding to a HOST TARGET binding site such as BINDING SITE I, BINDING SITE II or BINDING SITE III. Table 2 illustrates the complementarity of the nucleotide sequences of LOC119548 BINDING SITE, designated SEQ ID:2983, to the nucleotide sequence of VGAM131 RNA, herein designated VGAM RNA, also designated SEQ ID:466.

Another function of VGAM131 is therefore inhibition of LOC119548 (Accession XM_{—}058404). Accordingly, utilities of VGAM131 include diagnosis, prevention and treatment of diseases and clinical conditions associated with LOC119548. LOC120400 (Accession XM_{—}061971) is another VGAM131 host target gene. LOC120400 BINDING SITE is HOST TARGET binding site found in the 5′ untranslated region of mRNA encoded by LOC120400, corresponding to a HOST TARGET binding site such as BINDING SITE I, BINDING SITE II or BINDING SITE III. Table 2 illustrates the complementarity of the nucleotide sequences of LOC120400 BINDING SITE, designated SEQ ID:3027, to the nucleotide sequence of VGAM131 RNA, herein designated VGAM RNA, also designated SEQ ID:466.

Another function of VGAM131 is therefore inhibition of LOC120400 (Accession XM_{—}061971). Accordingly, utilities of VGAM131 include diagnosis, prevention and treatment of diseases and clinical conditions associated with LOC120400. FIG. 1 further provides a conceptual description of a novel bioinformatically detected viral gene of the present invention, referred to here as Viral Genomic Address Messenger 132 (VGAM132) viral gene, which modulates expression of respective host target genes thereof, the function and utility of which host target genes is known in the art.

VGAM132 is a novel bioinformatically detected regulatory, non protein coding, viral micro RNA (miRNA) gene. The method by which VGAM132 was detected is described hereinabove with reference to FIGS. 1-8.

VGAM132 gene, herein designated VGAM GENE, is a viral gene contained in the genome of Vaccinia Virus. VGAM132 host target gene, herein designated VGAM HOST TARGET GENE, is a human gene contained in the human genome.

VGAM132 gene encodes a VGAM132 precursor RNA, herein designated VGAM PRECURSOR RNA. Similar to other miRNA genes, and unlike most ordinary genes, VGAM132 precursor RNA does not encode a protein. A nucleotide sequence identical or highly similar to the nucleotide sequence of VGAM132 precursor RNA is designated SEQ ID:118, and is provided hereinbelow with reference to the sequence listing part. Nucleotide sequence SEQ ID:118 is located at position 60420 relative to the genome of Vaccinia Virus.

VGAM132 precursor RNA folds onto itself, forming VGAM132 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM132 folded precursor RNA into VGAM132 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM132 RNA is designated SEQ ID:467, and is provided hereinbelow with reference to the sequence listing part.

VGAM132 host target gene, herein designated VGAM HOST TARGET GENE, encodes a corresponding messenger RNA, VGAM132 host target RNA, herein designated VGAM HOST TARGET RNA. VGAM132 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM132 RNA, herein designated VGAM RNA, binds complementarily to one or more host target binding sites located in untranslated regions of VGAM132 host target RNA, herein designated VGAM HOST TARGET RNA. This complementary binding is due to the fact that the nucleotide sequence of VGAM132 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM132 RNA, herein designated VGAM RNA, may have a different number of host target binding sites in untranslated regions of a VGAM132 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM132 RNA, herein designated VGAM RNA, to host target binding sites on VGAM132 host target RNA, herein designated VGAM HOST TARGET RNA, such as BINDING SITE I, BINDING SITE II and BINDING SITE III, inhibits translation of VGAM132 host target RNA into VGAM132 host target protein, herein designated VGAM HOST TARGET PROTEIN. VGAM host target protein is therefore outlined by a broken line.

It is appreciated that VGAM132 host target gene, herein designated VGAM HOST TARGET GENE, in fact represents a plurality of VGAM132 host target genes. The mRNA of each one of this plurality of VGAM132 host target genes comprises one or more host target binding sites, each having a nucleotide sequence which is at least partly complementary to VGAM132 RNA, herein designated VGAM RNA, and which when bound by VGAM132 RNA causes inhibition of translation of respective one or more VGAM132 host target proteins.

It is further appreciated by one skilled in the art that the mode of translational inhibition illustrated by FIG. 1 with specific reference to translational inhibition exerted by VGAM132 gene, herein designated VGAM GENE, on one or more VGAM132 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM132 is inhibition of expression of host target genes, as part of a novel viral mechanism of attacking a host. Accordingly, utilities of VGAM132 include diagnosis, prevention and treatment of viral infection by Vaccinia Virus. Specific functions, and accordingly utilities, of VGAM132 correlate with, and may be deduced from, the identity of the host target genes which VGAM132 binds and inhibits, and the function of these host target genes, as elaborated hereinbelow.

Nucleotide sequences of the VGAM132 precursor RNA, herein designated VGAM PRECURSOR RNA, and of the ‘diced’ VGAM132 RNA, herein designated VGAM RNA, and a schematic representation of the secondary folding of VGAM132 folded precursor RNA, herein designated VGAM FOLDED PRECURSOR RNA, of VGAM132 are further described hereinbelow with reference to Table 1.

Nucleotide sequences of host target binding sites, such as BINDING SITE-I, BINDING SITE-II and BINDING SITE-III of FIG. 1, found on VGAM132 host target RNA, and schematic representation of the complementarity of each of these host target binding sites to VGAM132 RNA, herein designated VGAM RNA, are described hereinbelow with reference to Table 2.

As mentioned hereinabove with reference to FIG. 1, a function of VGAM132 gene, herein designated VGAM is inhibition of expression of VGAM132 target genes. It is appreciated that specific functions, and accordingly utilities, of VGAM132 correlate with, and may be deduced from, the identity of the target genes which VGAM132 binds and inhibits, and the function of these target genes, as elaborated hereinbelow.

Asparaginyl-tRNA Synthetase (NARS, Accession NM_{—}004539) is a VGAM132 host target gene. NARS BINDING SITE is HOST TARGET binding site found in the 3′ untranslated region of mRNA encoded by NARS, corresponding to a HOST TARGET binding site such as BINDING SITE I, BINDING SITE II or BINDING SITE III. Table 2 illustrates the complementarity of the nucleotide sequences of NARS BINDING SITE, designated SEQ ID:1125, to the nucleotide sequence of VGAM132 RNA, herein designated VGAM RNA, also designated SEQ ID:467.

A function of VGAM132 is therefore inhibition of Asparaginyl-tRNA Synthetase (NARS, Accession NM_{—}004539), a gene which is ASPARAGINYL-tRNA SYNTHETASE. Accordingly, utilities of VGAM132 include diagnosis, prevention and treatment of diseases and clinical conditions associated with NARS. The function of NARS has been established by previous studies. Using a DNA probe in human-rodent hybrid cells, Shows (1983) found that asparaginyl-tRNA synthetase segregated with peptidase A, a chromosome 18 marker. Cirullo et al. (1983) used the abbreviation-symbol ‘asnS.’ They isolated hybrids between human peripheral leukocytes and a temperature-sensitive CHO cell line with a thermolabile asparaginyl-tRNA synthetase. Hybrids selected at 39 degrees C required the presence of human chromosome 18. Temperature-resistant hybrid cells contained 2 forms of ASNRS: 1 highly thermal resistant, like the human enzyme, and 1 highly thermolabile, like the CHO mutant enzyme.

Full details of the abovementioned studies are described in the following publications, the disclosure of which are hereby incorporated by reference:
Cirullo, R. E.; Arredondo-Vega, F. X.; Smith, M.; Wasmuth, J. J.: Isolation and characterization of interspecific heat-resistant hybrids between a temperature-sensitive Chinese hamster cell asparaginyl-tRNA synthetase mutant and normal human leukocytes: assignment of human asnS gene to chromosome 18. Somat. Cell Genet. 9: 215-233, 1983.; and
Shows, T. B.: Personal Communication. Buffalo, N.Y., Jan. 11, 1983.

Further studies establishing the function and utilities of NARS are found in John Hopkins OMIM database record ID 108410, and in sited publications numbered 327-328 listed in the bibliography section hereinbelow, which are also hereby incorporated by reference. Bifunctional Apoptosis Regulator (BFAR, Accession XM_{—}027311) is another VGAM132 host target gene. BFAR BINDING SITE is HOST TARGET binding site found in the 3′ untranslated region of mRNA encoded by BFAR, corresponding to a HOST TARGET binding site such as BINDING SITE I, BINDING SITE II or BINDING SITE III. Table 2 illustrates the complementarity of the nucleotide sequences of BFAR BINDING SITE, designated SEQ ID:2581, to the nucleotide sequence of VGAM132 RNA, herein designated VGAM RNA, also designated SEQ ID:467.

Another function of VGAM132 is therefore inhibition of Bifunctional Apoptosis Regulator (BFAR, Accession XM_{—}027311). Accordingly, utilities of VGAM132 include diagnosis, prevention and treatment of diseases and clinical conditions associated with BFAR. HSNOV1 (Accession NM_{—}017515) is another VGAM132 host target gene. HSNOV1 BINDING SITE is HOST TARGET binding site found in the 3′ untranslated region of mRNA encoded by HSNOV1, corresponding to a HOST TARGET binding site such as BINDING SITE I, BINDING SITE II or BINDING SITE III. Table 2 illustrates the complementarity of the nucleotide sequences of HSNOV1 BINDING SITE, designated SEQ ID:1720, to the nucleotide sequence of VGAM132 RNA, herein designated VGAM RNA, also designated SEQ ID:467.

Another function of VGAM132 is therefore inhibition of HSNOV1 (Accession NM_{—}017515). Accordingly, utilities of VGAM132 include diagnosis, prevention and treatment of diseases and clinical conditions associated with HSNOV1. KIAA0628 (Accession NM_{—}014789) is another VGAM132 host target gene. KIAA0628 BINDING SITE is HOST TARGET binding site found in the 3′ untranslated region of mRNA encoded by KIAA0628, corresponding to a HOST TARGET binding site such as BINDING SITE I, BINDING SITE II or BINDING SITE III. Table 2 illustrates the complementarity of the nucleotide sequences of KIAA0628 BINDING SITE, designated SEQ ID:1547, to the nucleotide sequence of VGAM132 RNA, herein designated VGAM RNA, also designated SEQ ID:467.

Another function of VGAM132 is therefore inhibition of KIAA0628 (Accession NM_{—}014789). Accordingly, utilities of VGAM132 include diagnosis, prevention and treatment of diseases and clinical conditions associated with KIAA0628. KIAA1393 (Accession XM_{—}050793) is another VGAM132 host target gene. KIAA1393 BINDING SITE is HOST TARGET binding site found in the 3′ untranslated region of mRNA encoded by KIAA1393, corresponding to a HOST TARGET binding site such as BINDING SITE I, BINDING SITE II or BINDING SITE III. Table 2 illustrates the complementarity of the nucleotide sequences of KIAA1393 BINDING SITE, designated SEQ ID:2936, to the nucleotide sequence of VGAM132 RNA, herein designated VGAM RNA, also designated SEQ ID:467.

Another function of VGAM132 is therefore inhibition of KIAA1393 (Accession XM_{—}050793). Accordingly, utilities of VGAM132 include diagnosis, prevention and treatment of diseases and clinical conditions associated with KIAA1393. LOC147054 (Accession XM_{—}097172) is another VGAM132 host target gene. LOC147054 BINDING SITE is HOST TARGET binding site found in the 5′ untranslated region of mRNA encoded by LOC147054, corresponding to a HOST TARGET binding site such as BINDING SITE I, BINDING SITE II or BINDING SITE III. Table 2 illustrates the complementarity of the nucleotide sequences of LOC147054 BINDING SITE, designated SEQ ID:3289, to the nucleotide sequence of VGAM132 RNA, herein designated VGAM RNA, also designated SEQ ID:467.

Another function of VGAM132 is therefore inhibition of LOC147054 (Accession XM_{—}097172). Accordingly, utilities of VGAM132 include diagnosis, prevention and treatment of diseases and clinical conditions associated with LOC147054. FIG. 1 further provides a conceptual description of a novel bioinformatically detected viral gene of the present invention, referred to here as Viral Genomic Address Messenger 133 (VGAM133) viral gene, which modulates expression of respective host target genes thereof, the function and utility of which host target genes is known in the art.

VGAM133 is a novel bioinformatically detected regulatory, non protein coding, viral micro RNA (miRNA) gene. The method by which VGAM133 was detected is described hereinabove with reference to FIGS. 1-8.

VGAM133 gene, herein designated VGAM GENE, is a viral gene contained in the genome of Vaccinia Virus. VGAM133 host target gene, herein designated VGAM HOST TARGET GENE, is a human gene contained in the human genome.

VGAM133 gene encodes a VGAM133 precursor RNA, herein designated VGAM PRECURSOR RNA. Similar to other miRNA genes, and unlike most ordinary genes, VGAM133 precursor RNA does not encode a protein. A nucleotide sequence identical or highly similar to the nucleotide sequence of VGAM133 precursor RNA is designated SEQ ID:119, and is provided hereinbelow with reference to the sequence listing part. Nucleotide sequence SEQ ID:119 is located at position 62117 relative to the genome of Vaccinia Virus.

VGAM133 precursor RNA folds onto itself, forming VGAM133 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM133 folded precursor RNA into VGAM133 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM133 RNA is designated SEQ ID:468, and is provided hereinbelow with reference to the sequence listing part.

VGAM133 host target gene, herein designated VGAM HOST TARGET GENE, encodes a corresponding messenger RNA, VGAM133 host target RNA, herein designated VGAM HOST TARGET RNA. VGAM133 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM133 RNA, herein designated VGAM RNA, binds complementarily to one or more host target binding sites located in untranslated regions of VGAM133 host target RNA, herein designated VGAM HOST TARGET RNA. This complementary binding is due to the fact that the nucleotide sequence of VGAM133 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM133 RNA, herein designated VGAM RNA, may have a different number of host target binding sites in untranslated regions of a VGAM133 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM133 RNA, herein designated VGAM RNA, to host target binding sites on VGAM133 host target RNA, herein designated VGAM HOST TARGET RNA, such as BINDING SITE I, BINDING SITE II and BINDING SITE III, inhibits translation of VGAM133 host target RNA into VGAM133 host target protein, herein designated VGAM HOST TARGET PROTEIN. VGAM host target protein is therefore outlined by a broken line.

It is appreciated that VGAM133 host target gene, herein designated VGAM HOST TARGET GENE, in fact represents a plurality of VGAM133 host target genes. The mRNA of each one of this plurality of VGAM133 host target genes comprises one or more host target binding sites, each having a nucleotide sequence which is at least partly complementary to VGAM133 RNA, herein designated VGAM RNA, and which when bound by VGAM133 RNA causes inhibition of translation of respective one or more VGAM133 host target proteins.

It is further appreciated by one skilled in the art that the mode of translational inhibition illustrated by FIG. 1 with specific reference to translational inhibition exerted by VGAM133 gene, herein designated VGAM GENE, on one or more VGAM133 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM133 is inhibition of expression of host target genes, as part of a novel viral mechanism of attacking a host. Accordingly, utilities of VGAM133 include diagnosis, prevention and treatment of viral infection by Vaccinia Virus. Specific functions, and accordingly utilities, of VGAM133 correlate with, and may be deduced from, the identity of the host target genes which VGAM133 binds and inhibits, and the function of these host target genes, as elaborated hereinbelow.

Nucleotide sequences of the VGAM133 precursor RNA, herein designated VGAM PRECURSOR RNA, and of the ‘diced’ VGAM133 RNA, herein designated VGAM RNA, and a schematic representation of the secondary folding of VGAM133 folded precursor RNA, herein designated VGAM FOLDED PRECURSOR RNA, of VGAM133 are further described hereinbelow with reference to Table 1.

Nucleotide sequences of host target binding sites, such as BINDING SITE-I, BINDING SITE-II and BINDING SITE-III of FIG. 1, found on VGAM133 host target RNA, and schematic representation of the complementarity of each of these host target binding sites to VGAM133 RNA, herein designated VGAM RNA, are described hereinbelow with reference to Table 2.

As mentioned hereinabove with reference to FIG. 1, a function of VGAM133 gene, herein designated VGAM is inhibition of expression of VGAM133 target genes. It is appreciated that specific functions, and accordingly utilities, of VGAM133 correlate with, and may be deduced from, the identity of the target genes which VGAM133 binds and inhibits, and the function of these target genes, as elaborated hereinbelow.

TRIM (Accession NM_{—}016388) is a VGAM133 host target gene. TRIM BINDING SITE is HOST TARGET binding site found in the 3′ untranslated region of mRNA encoded by TRIM, corresponding to a HOST TARGET binding site such as BINDING SITE I, BINDING SITE II or BINDING SITE III. Table 2 illustrates the complementarity of the nucleotide sequences of TRIM BINDING SITE, designated SEQ ID:1684, to the nucleotide sequence of VGAM133 RNA, herein designated VGAM RNA, also designated SEQ ID:468.

A function of VGAM133 is therefore inhibition of TRIM (Accession NM_{—}016388), a gene which plays a role in recruiting signaling proteins to the plasma membrane upon T-cell receptor (TCR) complex activation in T cells. Accordingly, utilities of VGAM133 include diagnosis, prevention and treatment of diseases and clinical conditions associated with TRIM. The function of TRIM has been established by previous studies. T-cell activation requires stimulation of the T-cell receptor (TCR; OMIM Ref. No. 186880)-CD3 (see OMIM Ref. No. CD3Z; 186780) complex, followed by recruitment of an array of intracellular signaling proteins (e.g., GRB2 (OMIM Ref. No. 108355) and PLCG1 (OMIM Ref. No. 172420)). Mediating the interaction between the extracellular receptors and intracellular signaling pathways are adaptor proteins such as LAT (OMIM Ref. No. 602354). Bruyns et al. (1998) purified a 29/30-kD disulfide-linked dimeric phosphoprotein, which they called TRIM (TCR-interacting molecule), that associates and comodulates with the TCR-CD3 complex in T lymphocytes. By tryptic peptide sequence analysis and touchdown PCR analysis of a T-cell cDNA library, they isolated a cDNA encoding TRIM. Sequence analysis predicted that TRIM is a 186-amino acid type III transmembrane protein containing an 8-amino acid extracellular domain, which includes a cys residue, and a 19-amino acid transmembrane region that lacks charged residues. The intracellular portion possesses 4 potential phosphorylation sites and 8 tyrosine residues, at least 3 of which may be involved in Src (OMIM Ref. No. 190090) homology 2 (SH2)-mediated interactions with other signaling proteins. Northern blot analysis detected preferential expression of an approximately 2.0-kb TRIM transcript in thymus, with weaker expression in spleen, lymph nodes, and peripheral blood leukocytes. Western blot analysis of hematopoietic cell lines detected TRIM protein in T cell lines and, to a lesser extent, in natural killer cell lines, but not in B cell lines or in a monocytic cell line. Immunofluorescence and Western blot analyses showed that TRIM is localized in the cell membrane and is associated with CD3E (OMIM Ref. No. 186830) and CD3Z. The authors found that after T-cell activation, TRIM is phosphorylated by Src kinases on tyrosine residues, then associates with PIK3R1 (OMIM Ref. No. 171833).

Full details of the abovementioned studies are described in the following publications, the disclosure of which are hereby incorporated by reference:
Bruyns, E.; Marie-Cardine, A.; Kirchgessner, H.; Sagolla, K.; Shevchenko, A.; Mann, M.; Autschbach, F.; Bensussan, A.; Meuer, S.; Schraven, B.: T cell receptor (TCR) interacting molecule (TRIM), a novel disulfide-linked dimer associated with the TCR-CD3-zeta complex, recruits intracellular signaling proteins to the plasma membrane. J. Exp. Med. 188: 561-575, 1998.; and
Hubener, C.; Mincheva, A.; Lichter, P.; Schraven, B.; Bruyns, E.: Genomic organization and chromosomal localization of the human gene encoding the T-cell receptor-interacting molecule.

Further studies establishing the function and utilities of TRIM are found in John Hopkins OMIM database record ID 604962, and in sited publications numbered 1132 listed in the bibliography section hereinbelow, which are also hereby incorporated by reference. MGC11296 (Accession NM_{—}032352) is another VGAM133 host target gene. MGC11296 BINDING SITE is HOST TARGET binding site found in the 3′ untranslated region of mRNA encoded by MGC11296, corresponding to a HOST TARGET binding site such as BINDING SITE I, BINDING SITE II or BINDING SITE III. Table 2 illustrates the complementarity of the nucleotide sequences of MGC11296 BINDING SITE, designated SEQ ID:2248, to the nucleotide sequence of VGAM133 RNA, herein designated VGAM RNA, also designated SEQ ID:468.

Another function of VGAM133 is therefore inhibition of MGC11296 (Accession NM_{—}032352). Accordingly, utilities of VGAM133 include diagnosis, prevention and treatment of diseases and clinical conditions associated with MGC11296. PRO2037 (Accession NM_{—}018616) is another VGAM133 host target gene. PRO2037 BINDING SITE is HOST TARGET binding site found in the 5′ untranslated region of mRNA encoded by PRO2037, corresponding to a HOST TARGET binding site such as BINDING SITE I, BINDING SITE II or BINDING SITE III. Table 2 illustrates the complementarity of the nucleotide sequences of PRO2037 BINDING SITE, designated SEQ ID:1853, to the nucleotide sequence of VGAM133 RNA, herein designated VGAM RNA, also designated SEQ ID:468.

Another function of VGAM133 is therefore inhibition of PRO2037 (Accession NM_{—}018616). Accordingly, utilities of VGAM133 include diagnosis, prevention and treatment of diseases and clinical conditions associated with PRO2037. FIG. 1 further provides a conceptual description of a novel bioinformatically detected viral gene of the present invention, referred to here as Viral Genomic Address Messenger 134 (VGAM134) viral gene, which modulates expression of respective host target genes thereof, the function and utility of which host target genes is known in the art.

VGAM134 is a novel bioinformatically detected regulatory, non protein coding, viral micro RNA (miRNA) gene. The method by which VGAM134 was detected is described hereinabove with reference to FIGS. 1-8.

VGAM134 gene, herein designated VGAM GENE, is a viral gene contained in the genome of Vaccinia Virus. VGAM134 host target gene, herein designated VGAM HOST TARGET GENE, is a human gene contained in the human genome.

VGAM134 gene encodes a VGAM134 precursor RNA, herein designated VGAM PRECURSOR RNA. Similar to other miRNA genes, and unlike most ordinary genes, VGAM134 precursor RNA does not encode a protein. A nucleotide sequence identical or highly similar to the nucleotide sequence of VGAM134 precursor RNA is designated SEQ ID:120, and is provided hereinbelow with reference to the sequence listing part. Nucleotide sequence SEQ ID:120 is located at position 62005 relative to the genome of Vaccinia Virus.

VGAM134 precursor RNA folds onto itself, forming VGAM134 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM134 folded precursor RNA into VGAM134 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM134 RNA is designated SEQ ID:469, and is provided hereinbelow with reference to the sequence listing part.

VGAM134 host target gene, herein designated VGAM HOST TARGET GENE, encodes a corresponding messenger RNA, VGAM134 host target RNA, herein designated VGAM HOST TARGET RNA. VGAM134 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM134 RNA, herein designated VGAM RNA, binds complementarily to one or more host target binding sites located in untranslated regions of VGAM134 host target RNA, herein designated VGAM HOST TARGET RNA. This complementary binding is due to the fact that the nucleotide sequence of VGAM134 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM134 RNA, herein designated VGAM RNA, may have a different number of host target binding sites in untranslated regions of a VGAM134 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM134 RNA, herein designated VGAM RNA, to host target binding sites on VGAM134 host target RNA, herein designated VGAM HOST TARGET RNA, such as BINDING SITE I, BINDING SITE II and BINDING SITE III, inhibits translation of VGAM134 host target RNA into VGAM134 host target protein, herein designated VGAM HOST TARGET PROTEIN. VGAM host target protein is therefore outlined by a broken line.

It is appreciated that VGAM134 host target gene, herein designated VGAM HOST TARGET GENE, in fact represents a plurality of VGAM134 host target genes. The mRNA of each one of this plurality of VGAM134 host target genes comprises one or more host target binding sites, each having a nucleotide sequence which is at least partly complementary to VGAM134 RNA, herein designated VGAM RNA, and which when bound by VGAM134 RNA causes inhibition of translation of respective one or more VGAM134 host target proteins.

It is further appreciated by one skilled in the art that the mode of translational inhibition illustrated by FIG. 1 with specific reference to translational inhibition exerted by VGAM134 gene, herein designated VGAM GENE, on one or more VGAM134 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM134 is inhibition of expression of host target genes, as part of a novel viral mechanism of attacking a host. Accordingly, utilities of VGAM134 include diagnosis, prevention and treatment of viral infection by Vaccinia Virus. Specific functions, and accordingly utilities, of VGAM134 correlate with, and may be deduced from, the identity of the host target genes which VGAM134 binds and inhibits, and the function of these host target genes, as elaborated hereinbelow.

Nucleotide sequences of the VGAM134 precursor RNA, herein designated VGAM PRECURSOR RNA, and of the ‘diced’ VGAM134 RNA, herein designated VGAM RNA, and a schematic representation of the secondary folding of VGAM134 folded precursor RNA, herein designated VGAM FOLDED PRECURSOR RNA, of VGAM134 are further described hereinbelow with reference to Table 1.

Nucleotide sequences of host target binding sites, such as BINDING SITE-I, BINDING SITE-II and BINDING SITE-III of FIG. 1, found on VGAM134 host target RNA, and schematic representation of the complementarity of each of these host target binding sites to VGAM134 RNA, herein designated VGAM RNA, are described hereinbelow with reference to Table 2.

As mentioned hereinabove with reference to FIG. 1, a function of VGAM134 gene, herein designated VGAM is inhibition of expression of VGAM134 target genes. It is appreciated that specific functions, and accordingly utilities, of VGAM134 correlate with, and may be deduced from, the identity of the target genes which VGAM134 binds and inhibits, and the function of these target genes, as elaborated hereinbelow.

Paired Mesoderm Homeo Box 1 (PMX1, Accession NM_{—}022716) is a VGAM134 host target gene. PMX1 BINDING SITE is HOST TARGET binding site found in the 3′ untranslated region of mRNA encoded by PMX1, corresponding to a HOST TARGET binding site such as BINDING SITE I, BINDING SITE II or BINDING SITE III. Table 2 illustrates the complementarity of the nucleotide sequences of PMX1 BINDING SITE, designated SEQ ID:1996, to the nucleotide sequence of VGAM134 RNA, herein designated VGAM RNA, also designated SEQ ID:469.

A function of VGAM134 is therefore inhibition of Paired Mesoderm Homeo Box 1 (PMX1, Accession NM_{—}022716), a gene which acts as a transcriptional regulator of muscle creatine kinase. Accordingly, utilities of VGAM134 include diagnosis, prevention and treatment of diseases and clinical conditions associated with PMX1. The function of PMX1 has been established by previous studies. Homeo box genes are expressed in specific temporal and spatial patterns and function as transcriptional regulators of developmental processes. The murine homeo box gene Pmx (paired mesoderm homeo box), previously called K-2 and mHox, is expressed in a mesodermally restricted pattern in embryos and most abundantly in cardiac, skeletal, and smooth muscle tissues in adults (Kern et al., 1994). Grueneberg et al. (1992) cloned the homologous human gene. By means of interspecific backcross analysis, Kern et al. (1994) determined that the Pmx gene is located on mouse chromosome 1, approximately 3.3 cM distal to the Gsh-4 homeo box locus. The gene contains at least 5 exons spanning a minimum of 60 kb of genomic DNA, making this the largest known murine homeo box gene. The homologous human gene may map to 1q inasmuch as this region is syntenic with the region of mouse chromosome 1 where Pmx is located. Norris et al. (2000) mapped the human PRRX1 gene to 1q23 by fluorescence in situ hybridization.

Full details of the abovementioned studies are described in the following publications, the disclosure of which are hereby incorporated by reference:
Grueneberg, D. A.; Natesan, S.; Alexandre, C.; Gilman, M. Z.: Human and Drosophila homeodomain proteins that enhance the DNA-binding activity of serum response factor. Science 257: 1089-1095, 1992.; and
Norris, R. A.; Scott, K. K.; Moore, C. S.; Stetten, G.; Brown, C. R.; Jabs, E. W.; Wulfsberg, E. A.; Yu, J.; Kern, M. J.: Human PRRX1 and PRRX2 genes: cloning, expression, genomic localiz.

Further studies establishing the function and utilities of PMX1 are found in John Hopkins OMIM database record ID 167420, and in sited publications numbered 2517-2521 listed in the bibliography section hereinbelow, which are also hereby incorporated by reference. FLJ22557 (Accession NM_{—}024713) is another VGAM134 host target gene. FLJ22557 BINDING SITE is HOST TARGET binding site found in the 3′ untranslated region of mRNA encoded by FLJ22557, corresponding to a HOST TARGET binding site such as BINDING SITE I, BINDING SITE II or BINDING SITE III. Table 2 illustrates the complementarity of the nucleotide sequences of FLJ22557 BINDING SITE, designated SEQ ID:2083, to the nucleotide sequence of VGAM134 RNA, herein designated VGAM RNA, also designated SEQ ID:469.

Another function of VGAM134 is therefore inhibition of FLJ22557 (Accession NM_{—}024713). Accordingly, utilities of VGAM134 include diagnosis, prevention and treatment of diseases and clinical conditions associated with FLJ22557. KIAA0618 (Accession NM_{—}014833) is another VGAM134 host target gene. KIAA0618 BINDING SITE is HOST TARGET binding site found in the 5′ untranslated region of mRNA encoded by KIAA0618, corresponding to a HOST TARGET binding site such as BINDING SITE I, BINDING SITE II or BINDING SITE III. Table 2 illustrates the complementarity of the nucleotide sequences of KIAA0618 BINDING SITE, designated SEQ ID:1563, to the nucleotide sequence of VGAM134 RNA, herein designated VGAM RNA, also designated SEQ ID:469.

Another function of VGAM134 is therefore inhibition of KIAA0618 (Accession NM_{—}014833). Accordingly, utilities of VGAM134 include diagnosis, prevention and treatment of diseases and clinical conditions associated with KIAA0618. LOC154215 (Accession XM_{—}087875) is another VGAM134 host target gene. LOC154215 BINDING SITE is HOST TARGET binding site found in the 3′ untranslated region of mRNA encoded by LOC154215, corresponding to a HOST TARGET binding site such as BINDING SITE I, BINDING SITE II or BINDING SITE III. Table 2 illustrates the complementarity of the nucleotide sequences of LOC154215 BINDING SITE, designated SEQ ID:3188, to the nucleotide sequence of VGAM134 RNA, herein designated VGAM RNA, also designated SEQ ID:469.

Another function of VGAM134 is therefore inhibition of LOC154215 (Accession XM_{—}087875). Accordingly, utilities of VGAM134 include diagnosis, prevention and treatment of diseases and clinical conditions associated with LOC154215. FIG. 1 further provides a conceptual description of a novel bioinformatically detected viral gene of the present invention, referred to here as Viral Genomic Address Messenger 135 (VGAM135) viral gene, which modulates expression of respective host target genes thereof, the function and utility of which host target genes is known in the art.

VGAM135 is a novel bioinformatically detected regulatory, non protein coding, viral micro RNA (miRNA) gene. The method by which VGAM135 was detected is described hereinabove with reference to FIGS. 1-8.

VGAM135 gene, herein designated VGAM GENE, is a viral gene contained in the genome of Vaccinia Virus. VGAM135 host target gene, herein designated VGAM HOST TARGET GENE, is a human gene contained in the human genome.

VGAM135 gene encodes a VGAM135 precursor RNA, herein designated VGAM PRECURSOR RNA. Similar to other miRNA genes, and unlike most ordinary genes, VGAM135 precursor RNA does not encode a protein. A nucleotide sequence identical or highly similar to the nucleotide sequence of VGAM135 precursor RNA is designated SEQ ID:121, and is provided hereinbelow with reference to the sequence listing part. Nucleotide sequence SEQ ID:121 is located at position 64481 relative to the genome of Vaccinia Virus.

VGAM135 precursor RNA folds onto itself, forming VGAM135 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM135 folded precursor RNA into VGAM135 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM135 RNA is designated SEQ ID:470, and is provided hereinbelow with reference to the sequence listing part.

VGAM135 host target gene, herein designated VGAM HOST TARGET GENE, encodes a corresponding messenger RNA, VGAM135 host target RNA, herein designated VGAM HOST TARGET RNA. VGAM135 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM135 RNA, herein designated VGAM RNA, binds complementarily to one or more host target binding sites located in untranslated regions of VGAM135 host target RNA, herein designated VGAM HOST TARGET RNA. This complementary binding is due to the fact that the nucleotide sequence of VGAM135 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM135 RNA, herein designated VGAM RNA, may have a different number of host target binding sites in untranslated regions of a VGAM135 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM135 RNA, herein designated VGAM RNA, to host target binding sites on VGAM135 host target RNA, herein designated VGAM HOST TARGET RNA, such as BINDING SITE I, BINDING SITE II and BINDING SITE III, inhibits translation of VGAM135 host target RNA into VGAM135 host target protein, herein designated VGAM HOST TARGET PROTEIN. VGAM host target protein is therefore outlined by a broken line.

It is appreciated that VGAM135 host target gene, herein designated VGAM HOST TARGET GENE, in fact represents a plurality of VGAM135 host target genes. The mRNA of each one of this plurality of VGAM135 host target genes comprises one or more host target binding sites, each having a nucleotide sequence which is at least partly complementary to VGAM135 RNA, herein designated VGAM RNA, and which when bound by VGAM135 RNA causes inhibition of translation of respective one or more VGAM135 host target proteins.

It is further appreciated by one skilled in the art that the mode of translational inhibition illustrated by FIG. 1 with specific reference to translational inhibition exerted by VGAM135 gene, herein designated VGAM GENE, on one or more VGAM135 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM135 is inhibition of expression of host target genes, as part of a novel viral mechanism of attacking a host. Accordingly, utilities of VGAM135 include diagnosis, prevention and treatment of viral infection by Vaccinia Virus. Specific functions, and accordingly utilities, of VGAM135 correlate with, and may be deduced from, the identity of the host target genes which VGAM135 binds and inhibits, and the function of these host target genes, as elaborated hereinbelow.

Nucleotide sequences of the VGAM135 precursor RNA, herein designated VGAM PRECURSOR RNA, and of the ‘diced’ VGAM135 RNA, herein designated VGAM RNA, and a schematic representation of the secondary folding of VGAM135 folded precursor RNA, herein designated VGAM FOLDED PRECURSOR RNA, of VGAM135 are further described hereinbelow with reference to Table 1.

Nucleotide sequences of host target binding sites, such as BINDING SITE-I, BINDING SITE-II and BINDING SITE-III of FIG. 1, found on VGAM135 host target RNA, and schematic representation of the complementarity of each of these host target binding sites to VGAM135 RNA, herein designated VGAM RNA, are described hereinbelow with reference to Table 2.

As mentioned hereinabove with reference to FIG. 1, a function of VGAM135 gene, herein designated VGAM is inhibition of expression of VGAM135 target genes. It is appreciated that specific functions, and accordingly utilities, of VGAM135 correlate with, and may be deduced from, the identity of the target genes which VGAM135 binds and inhibits, and the function of these target genes, as elaborated hereinbelow.

Glycoprotein A Repetitions Predominant (GARP, Accession NM_{—}005512) is a VGAM135 host target gene. GARP BINDING SITE is HOST TARGET binding site found in the 3′ untranslated region of mRNA encoded by GARP, corresponding to a HOST TARGET binding site such as BINDING SITE I, BINDING SITE II or BINDING SITE III. Table 2 illustrates the complementarity of the nucleotide sequences of GARP BINDING SITE, designated SEQ ID:1220, to the nucleotide sequence of VGAM135 RNA, herein designated VGAM RNA, also designated SEQ ID:470.

A function of VGAM135 is therefore inhibition of Glycoprotein A Repetitions Predominant (GARP, Accession NM_{—}005512). Accordingly, utilities of VGAM135 include diagnosis, prevention and treatment of diseases and clinical conditions associated with GARP. TEM8 (Accession NM_{—}018153) is another VGAM135 host target gene. TEM8 BINDING SITE is HOST TARGET binding site found in the 3′ untranslated region of mRNA encoded by TEM8, corresponding to a HOST TARGET binding site such as BINDING SITE I, BINDING SITE II or BINDING SITE III. Table 2 illustrates the complementarity of the nucleotide sequences of TEM8 BINDING SITE, designated SEQ ID:1790, to the nucleotide sequence of VGAM135 RNA, herein designated VGAM RNA, also designated SEQ ID:470.

Another function of VGAM135 is therefore inhibition of TEM8 (Accession NM_{—}018153), a gene which is a tumor-specific endothelial marker. Accordingly, utilities of VGAM135 include diagnosis, prevention and treatment of diseases and clinical conditions associated with TEM8. The function of TEM8 has been established by previous studies. St. Croix et al. (2000) compared gene expression patterns of endothelial cells derived from blood vessels of normal and malignant colorectal tissues to identify genes involved in tumor angiogenesis. Among the genes they identified was TEM8, which encodes a 564-amino acid protein. Bradley et al. (2001) isolated a cDNA encoding ATR and determined that the first 364 amino acids of the 368-amino acid ATR protein are identical to those of TEM8. The C-terminal ends of the ATR and TEM8 proteins then diverge, presumably due to alternative splicing, such that ATR has a cytoplasmic tail of only 25 amino acids, whereas TEM8 has a cytoplasmic tail of 221 amino acids. (Bradley et al. (2001) noted in proof that another apparently full-length ATR/TEM8-related cDNA clone (GenBank BC01207) encodes a protein with yet another C-terminal end.) The ATR protein contains a 27-amino acid signal peptide; a 293-amino acid extracellular domain with 3 putative end-length glycosylation sites; and a 23-amino acid putative transmembrane region followed by the short cytoplasmic tail. An extracellular von Willebrand factor type A (VWA) domain is located between residues 44 and 216 of the ATR protein. The cytoplasmic tail of ATR contains an acidic cluster (EESEE) similar to a motif in the cytoplasmic tail of furin (OMIM Ref. No. 136950) that specifies basolateral sorting of this protease in polarized epithelial cells. The mouse homolog of ATR/TEM8 is highly related to the human clones, showing more than 98% sequence identity in the extracellular domain. ATR and/or TEM8 is expressed in a number of different tissues, including central nervous system, heart, lung, and lymphocytes. Bradley et al. (2001) confirmed that the VWA domain of ATR binds directly to the protective antigen of anthrax, suggesting that ATR may also function as a protective antigen receptor. They suggested that the finding that the soluble VWA domain of ATR inhibits toxin action, coupled with the use of the cloned receptor as a tool for identifying inhibitors of the protective antigen-receptor interaction, holds promise for the development of novel approaches for the treatment of anthrax.

Full details of the abovementioned studies are described in the following publications, the disclosure of which are hereby incorporated by reference:
St. Croix, B.; Rago, C.; Velculescu, V.; Traverso, G.; Romans, K. E.; Montgomery, E.; Lal, A.; Riggins, G. J.; Lengauer, C.; Vogelstein, B.; Kinzler, K. W.: Genes expressed in human tumor endothelium. Science 289: 1197-1202, 2000.; and
Bradley, K. A.; Mogridge, J.; Mourez, M.; Collier, R. J.; Young, J. A. T.: Identification of the cellular receptor for anthrax toxin. Nature 414: 160-161, 2001.

Further studies establishing the function and utilities of TEM8 are found in John Hopkins OMIM database record ID 606410, and in sited publications numbered 1013, 159 and 1598 listed in the bibliography section hereinbelow, which are also hereby incorporated by reference. Thyrotropin-releasing Hormone Receptor (TRHR, Accession NM_{—}003301) is another VGAM135 host target gene. TRHR BINDING SITE is HOST TARGET binding site found in the 3′ untranslated region of mRNA encoded by TRHR, corresponding to a HOST TARGET binding site such as BINDING SITE I, BINDING SITE II or BINDING SITE III. Table 2 illustrates the complementarity of the nucleotide sequences of TRHR BINDING SITE, designated SEQ ID:1009, to the nucleotide sequence of VGAM135 RNA, herein designated VGAM RNA, also designated SEQ ID:470.

Another function of VGAM135 is therefore inhibition of Thyrotropin-releasing Hormone Receptor (TRHR, Accession NM_{—}003301), a gene which isolated central hypothyroidism. Accordingly, utilities of VGAM135 include diagnosis, prevention and treatment of diseases and clinical conditions associated with TRHR. The function of TRHR has been established by previous studies. Thyrotropin-releasing hormone (TRH; 275120), a small neuropeptide, is widely distributed throughout the central and peripheral nervous system as well as in extraneural tissues. The peptide is synthesized in the hypothalamus and transported by the portal vascular system to the anterior pituitary where it acts on thyrotropic and lactotropic cells to promote secretion of TSH and prolactin, respectively. Thyrotropin-releasing hormone receptor is a G protein-coupled receptor that activates the inositol phospholipid-calcium-protein kinase C transduction pathway upon the binding of TRH. The TRHR gene is expressed in the thyrotrope cells of the anterior pituitary. Straub et al. (1990) isolated mouse Trhr cDNA from mouse pituitary thyrotropic tumor cells; the 3.8-kb cDNA encodes a protein of 393 amino acids. Zhao et al. (1992) found that the rat Trhr cDNA is 3.5 kb and encodes a protein of 412 amino acids. Matre et al. (1993) isolated a human TRHR cDNA from a human brain cDNA library. The predicted amino acid sequence of the human TRHR protein showed high homology with the rat and mouse proteins with the exception of the C-terminal region. The human gene was thought to contain 2 introns. By interspecific backcross analysis, Lossie et al. (1993) showed that the Trhr gene maps to mouse chromosome 15. They quoted work of Eidne et al. (1993) assigning the human TRHR gene to the long arm of chromosome 8 by in situ hybridization. This is consistent with the finding by Lossie et al. (1993) that the mouse gene maps to a region that is homologous to 8q and contains the MYC (OMIM Ref. No. 190080) and TG (OMIM Ref. No. 188450) genes, which are located in the human 8q24 region. By studying human/hamster somatic cell hybrids, Yamada et al. (1993) assigned the TRHR gene to chromosome 8. Morrison et al. (1994) isolated a cDNA for thyrotropin-releasing hormone receptor from a human pituitary cDNA library. By use of this cDNA as a biotinylated probe, they localized the TRHR gene to 8q23 by in situ hybridization.

Full details of the abovementioned studies are described in the following publications, the disclosure of which are hereby incorporated by reference:
Matre, V.; Karlsen, H. E.; Wright, M. S.; Lundell, I.; Fjeldheim, A. K.; Gabrielsen, O. S.; Larhammar, D.; Gautvik, K. M.: Molecular cloning of a functional human thyrotropin-releasing hormone receptor. Biochem. Biophys. Res. Commun. 195: 179-185, 1993.; and
Morrison, N.; Duthie, S. M.; Boyd, E.; Eidne, K. A.; Connor, J. M.: Assignment of the gene encoding the human thyrotropin-releasing hormone receptor to 8q23 by fluorescence in situ hybr.

Further studies establishing the function and utilities of TRHR are found in John Hopkins OMIM database record ID 188545, and in sited publications numbered 236 and 2449-2425 listed in the bibliography section hereinbelow, which are also hereby incorporated by reference. Leucine-rich Repeat LGI Family, Member 2 (LGI2, Accession NM_{—}018176) is another VGAM135 host target gene. LGI2 BINDING SITE is HOST TARGET binding site found in the 3′ untranslated region of mRNA encoded by LGI2, corresponding to a HOST TARGET binding site such as BINDING SITE I, BINDING SITE II or BINDING SITE III. Table 2 illustrates the complementarity of the nucleotide sequences of LGI2 BINDING SITE, designated SEQ ID:1792, to the nucleotide sequence of VGAM135 RNA, herein designated VGAM RNA, also designated SEQ ID:470.

Another function of VGAM135 is therefore inhibition of Leucine-rich Repeat LGI Family, Member 2 (LGI2, Accession NM_{—}018176). Accordingly, utilities of VGAM135 include diagnosis, prevention and treatment of diseases and clinical conditions associated with LGI2. MGC4638 (Accession NM_{—}031479) is another VGAM135 host target gene. MGC4638 BINDING SITE is HOST TARGET binding site found in the 3′ untranslated region of mRNA encoded by MGC4638, corresponding to a HOST TARGET binding site such as BINDING SITE I, BINDING SITE II or BINDING SITE III. Table 2 illustrates the complementarity of the nucleotide sequences of MGC4638 BINDING SITE, designated SEQ ID:2212, to the nucleotide sequence of VGAM135 RNA, herein designated VGAM RNA, also designated SEQ ID:470.

Another function of VGAM135 is therefore inhibition of MGC4638 (Accession NM_{—}031479). Accordingly, utilities of VGAM135 include diagnosis, prevention and treatment of diseases and clinical conditions associated with MGC4638. LOC139065 (Accession XM_{—}066456) is another VGAM135 host target gene. LOC139065 BINDING SITE is HOST TARGET binding site found in the 5′ untranslated region of mRNA encoded by LOC139065, corresponding to a HOST TARGET binding site such as BINDING SITE I, BINDING SITE II or BINDING SITE III. Table 2 illustrates the complementarity of the nucleotide sequences of LOC139065 BINDING SITE, designated SEQ ID:3035, to the nucleotide sequence of VGAM135 RNA, herein designated VGAM RNA, also designated SEQ ID:470.

Another function of VGAM135 is therefore inhibition of LOC139065 (Accession XM_{—}066456). Accordingly, utilities of VGAM135 include diagnosis, prevention and treatment of diseases and clinical conditions associated with LOC139065. LOC154282 (Accession XM_{—}098505) is another VGAM135 host target gene. LOC154282 BINDING SITE is HOST TARGET binding site found in the 3′ untranslated region of mRNA encoded by LOC154282, corresponding to a HOST TARGET binding site such as BINDING SITE I, BINDING SITE II or BINDING SITE III. Table 2 illustrates the complementarity of the nucleotide sequences of LOC154282 BINDING SITE, designated SEQ ID:3372, to the nucleotide sequence of VGAM135 RNA, herein designated VGAM RNA, also designated SEQ ID:470.

Another function of VGAM135 is therefore inhibition of LOC154282 (Accession XM_{—}098505). Accordingly, utilities of VGAM135 include diagnosis, prevention and treatment of diseases and clinical conditions associated with LOC154282. LOC91516 (Accession XM_{—}038924) is another VGAM135 host target gene. LOC91516 BINDING SITE is HOST TARGET binding site found in the 5′ untranslated region of mRNA encoded by LOC91516, corresponding to a HOST TARGET binding site such as BINDING SITE I, BINDING SITE II or BINDING SITE III. Table 2 illustrates the complementarity of the nucleotide sequences of LOC91516 BINDING SITE, designated SEQ ID:2745, to the nucleotide sequence of VGAM135 RNA, herein designated VGAM RNA, also designated SEQ ID:470.

Another function of VGAM135 is therefore inhibition of LOC91516 (Accession XM_{—}038924). Accordingly, utilities of VGAM135 include diagnosis, prevention and treatment of diseases and clinical conditions associated with LOC91516. FIG. 1 further provides a conceptual description of a novel bioinformatically detected viral gene of the present invention, referred to here as Viral Genomic Address Messenger 136 (VGAM136) viral gene, which modulates expression of respective host target genes thereof, the function and utility of which host target genes is known in the art.

VGAM136 is a novel bioinformatically detected regulatory, non protein coding, viral micro RNA (miRNA) gene. The method by which VGAM136 was detected is described hereinabove with reference to FIGS. 1-8.

VGAM136 gene, herein designated VGAM GENE, is a viral gene contained in the genome of Vaccinia Virus. VGAM136 host target gene, herein designated VGAM HOST TARGET GENE, is a human gene contained in the human genome.

VGAM136 gene encodes a VGAM136 precursor RNA, herein designated VGAM PRECURSOR RNA. Similar to other miRNA genes, and unlike most ordinary genes, VGAM136 precursor RNA does not encode a protein. A nucleotide sequence identical or highly similar to the nucleotide sequence of VGAM136 precursor RNA is designated SEQ ID:122, and is provided hereinbelow with reference to the sequence listing part. Nucleotide sequence SEQ ID:122 is located at position 65175 relative to the genome of Vaccinia Virus.

VGAM136 precursor RNA folds onto itself, forming VGAM136 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM136 folded precursor RNA into VGAM136 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM136 RNA is designated SEQ ID:471, and is provided hereinbelow with reference to the sequence listing part.

VGAM136 host target gene, herein designated VGAM HOST TARGET GENE, encodes a corresponding messenger RNA, VGAM136 host target RNA, herein designated VGAM HOST TARGET RNA. VGAM136 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM136 RNA, herein designated VGAM RNA, binds complementarily to one or more host target binding sites located in untranslated regions of VGAM136 host target RNA, herein designated VGAM HOST TARGET RNA. This complementary binding is due to the fact that the nucleotide sequence of VGAM136 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM136 RNA, herein designated VGAM RNA, may have a different number of host target binding sites in untranslated regions of a VGAM136 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM136 RNA, herein designated VGAM RNA, to host target binding sites on VGAM136 host target RNA, herein designated VGAM HOST TARGET RNA, such as BINDING SITE I, BINDING SITE II and BINDING SITE III, inhibits translation of VGAM136 host target RNA into VGAM136 host target protein, herein designated VGAM HOST TARGET PROTEIN. VGAM host target protein is therefore outlined by a broken line.

It is appreciated that VGAM136 host target gene, herein designated VGAM HOST TARGET GENE, in fact represents a plurality of VGAM136 host target genes. The mRNA of each one of this plurality of VGAM136 host target genes comprises one or more host target binding sites, each having a nucleotide sequence which is at least partly complementary to VGAM136 RNA, herein designated VGAM RNA, and which when bound by VGAM136 RNA causes inhibition of translation of respective one or more VGAM136 host target proteins.

It is further appreciated by one skilled in the art that the mode of translational inhibition illustrated by FIG. 1 with specific reference to translational inhibition exerted by VGAM136 gene, herein designated VGAM GENE, on one or more VGAM136 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM136 is inhibition of expression of host target genes, as part of a novel viral mechanism of attacking a host. Accordingly, utilities of VGAM136 include diagnosis, prevention and treatment of viral infection by Vaccinia Virus. Specific functions, and accordingly utilities, of VGAM136 correlate with, and may be deduced from, the identity of the host target genes which VGAM136 binds and inhibits, and the function of these host target genes, as elaborated hereinbelow.

Nucleotide sequences of the VGAM136 precursor RNA, herein designated VGAM PRECURSOR RNA, and of the ‘diced’ VGAM136 RNA, herein designated VGAM RNA, and a schematic representation of the secondary folding of VGAM136 folded precursor RNA, herein designated VGAM FOLDED PRECURSOR RNA, of VGAM136 are further described hereinbelow with reference to Table 1.

Nucleotide sequences of host target binding sites, such as BINDING SITE-I, BINDING SITE-II and BINDING SITE-III of FIG. 1, found on VGAM136 host target RNA, and schematic representation of the complementarity of each of these host target binding sites to VGAM136 RNA, herein designated VGAM RNA, are described hereinbelow with reference to Table 2.

As mentioned hereinabove with reference to FIG. 1, a function of VGAM136 gene, herein designated VGAM is inhibition of expression of VGAM136 target genes. It is appreciated that specific functions, and accordingly utilities, of VGAM136 correlate with, and may be deduced from, the identity of the target genes which VGAM136 binds and inhibits, and the function of these target genes, as elaborated hereinbelow.

Chromosome 20 Open Reading Frame 28 (C20orf28, Accession NM_{—}015417) is a VGAM136 host target gene. C20orf28 BINDING SITE is HOST TARGET binding site found in the 3′ untranslated region of mRNA encoded by C20orf28, corresponding to a HOST TARGET binding site such as BINDING SITE I, BINDING SITE II or BINDING SITE III. Table 2 illustrates the complementarity of the nucleotide sequences of C20orf28 BINDING SITE, designated SEQ ID: 1629, to the nucleotide sequence of VGAM136 RNA, herein designated VGAM RNA, also designated SEQ ID:471.

A function of VGAM136 is therefore inhibition of Chromosome 20 Open Reading Frame 28 (C20orf28, Accession NM_{—}015417). Accordingly, utilities of VGAM136 include diagnosis, prevention and treatment of diseases and clinical conditions associated with C20orf28. MGC5139 (Accession XM_{—}058587) is another VGAM136 host target gene. MGC5139 BINDING SITE is HOST TARGET binding site found in the 5′ untranslated region of mRNA encoded by MGC5139, corresponding to a HOST TARGET binding site such as BINDING SITE I, BINDING SITE II or BINDING SITE III. Table 2 illustrates the complementarity of the nucleotide sequences of MGC5139 BINDING SITE, designated SEQ ID:2994, to the nucleotide sequence of VGAM136 RNA, herein designated VGAM RNA, also designated SEQ ID:471.

Another function of VGAM136 is therefore inhibition of MGC5139 (Accession XM_{—}058587). Accordingly, utilities of VGAM136 include diagnosis, prevention and treatment of diseases and clinical conditions associated with MGC5139. PRO0899 (Accession NM_{—}018565) is another VGAM136 host target gene. PRO0899 BINDING SITE is HOST TARGET binding site found in the 3′ untranslated region of mRNA encoded by PRO0899, corresponding to a HOST TARGET binding site such as BINDING SITE I, BINDING SITE II or BINDING SITE III. Table 2 illustrates the complementarity of the nucleotide sequences of PRO0899 BINDING SITE, designated SEQ ID:1847, to the nucleotide sequence of VGAM136 RNA, herein designated VGAM RNA, also designated SEQ ID:471.

Another function of VGAM136 is therefore inhibition of PRO0899 (Accession NM_{—}018565). Accordingly, utilities of VGAM136 include diagnosis, prevention and treatment of diseases and clinical conditions associated with PRO0899. PRO1386 (Accession NM_{—}031269) is another VGAM136 host target gene. PRO1386 BINDING SITE is HOST TARGET binding site found in the 3′ untranslated region of mRNA encoded by PRO1386, corresponding to a HOST TARGET binding site such as BINDING SITE I, BINDING SITE II or BINDING SITE III. Table 2 illustrates the complementarity of the nucleotide sequences of PRO1386 BINDING SITE, designated SEQ ID:2187, to the nucleotide sequence of VGAM136 RNA, herein designated VGAM RNA, also designated SEQ ID:471.

Another function of VGAM136 is therefore inhibition of PRO1386 (Accession NM_{—}031269). Accordingly, utilities of VGAM136 include diagnosis, prevention and treatment of diseases and clinical conditions associated with PRO1386. Protein Tyrosine Phosphatase, Receptor Type, T (PTPRT, Accession NM_{—}133170) is another VGAM136 host target gene. PTPRT BINDING SITE is HOST TARGET binding site found in the 3′ untranslated region of mRNA encoded by PTPRT, corresponding to a HOST TARGET binding site such as BINDING SITE I, BINDING SITE II or BINDING SITE III. Table 2 illustrates the complementarity of the nucleotide sequences of PTPRT BINDING SITE, designated SEQ ID:2399, to the nucleotide sequence of VGAM136 RNA, herein designated VGAM RNA, also designated SEQ ID:471.

Another function of VGAM136 is therefore inhibition of Protein Tyrosine Phosphatase, Receptor Type, T (PTPRT, Accession NM_{—}133170). Accordingly, utilities of VGAM136 include diagnosis, prevention and treatment of diseases and clinical conditions associated with PTPRT. T-cell Leukemia/lymphoma 6 (TCL6, Accession NM_{—}020550) is another VGAM136 host target gene. TCL6 BINDING SITE1 and TCL6 BINDING SITE2 are HOST TARGET binding sites found in untranslated regions of mRNA encoded by TCL6, corresponding to HOST TARGET binding sites such as BINDING SITE I, BINDING SITE II or BINDING SITE III. Table 2 illustrates the complementarity of the nucleotide sequences of TCL6 BINDING SITE1 and TCL6 BINDING SITE2, designated SEQ ID:1919 and SEQ ID: 1433 respectively, to the nucleotide sequence of VGAM136 RNA, herein designated VGAM RNA, also designated SEQ ID:471.

Another function of VGAM136 is therefore inhibition of T-cell Leukemia/lymphoma 6 (TCL6, Accession NM_{—}020550). Accordingly, utilities of VGAM136 include diagnosis, prevention and treatment of diseases and clinical conditions associated with TCL6. LOC255759 (Accession XM_{—}173708) is another VGAM136 host target gene. LOC255759 BINDING SITE is HOST TARGET binding site found in the 5′ untranslated region of mRNA encoded by LOC255759, corresponding to a HOST TARGET binding site such as BINDING SITE I, BINDING SITE II or BINDING SITE III. Table 2 illustrates the complementarity of the nucleotide sequences of LOC255759 BINDING SITE, designated SEQ ID:3739, to the nucleotide sequence of VGAM136 RNA, herein designated VGAM RNA, also designated SEQ ID:471.

Another function of VGAM136 is therefore inhibition of LOC255759 (Accession XM_{—}173708). Accordingly, utilities of VGAM136 include diagnosis, prevention and treatment of diseases and clinical conditions associated with LOC255759. FIG. 1 further provides a conceptual description of a novel bioinformatically detected viral gene of the present invention, referred to here as Viral Genomic Address Messenger 137 (VGAM137) viral gene, which modulates expression of respective host target genes thereof, the function and utility of which host target genes is known in the art.

VGAM137 is a novel bioinformatically detected regulatory, non protein coding, viral micro RNA (miRNA) gene. The method by which VGAM137 was detected is described hereinabove with reference to FIGS. 1-8.

VGAM137 gene, herein designated VGAM GENE, is a viral gene contained in the genome of Vaccinia Virus. VGAM137 host target gene, herein designated VGAM HOST TARGET GENE, is a human gene contained in the human genome.

VGAM137 gene encodes a VGAM137 precursor RNA, herein designated VGAM PRECURSOR RNA. Similar to other miRNA genes, and unlike most ordinary genes, VGAM137 precursor RNA does not encode a protein. A nucleotide sequence identical or highly similar to the nucleotide sequence of VGAM137 precursor RNA is designated SEQ ID:123, and is provided hereinbelow with reference to the sequence listing part. Nucleotide sequence SEQ ID:123 is located at position 64691 relative to the genome of Vaccinia Virus.

VGAM137 precursor RNA folds onto itself, forming VGAM137 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM137 folded precursor RNA into VGAM137 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM137 RNA is designated SEQ ID:472, and is provided hereinbelow with reference to the sequence listing part.

VGAM137 host target gene, herein designated VGAM HOST TARGET GENE, encodes a corresponding messenger RNA, VGAM137 host target RNA, herein designated VGAM HOST TARGET RNA. VGAM137 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM137 RNA, herein designated VGAM RNA, binds complementarily to one or more host target binding sites located in untranslated regions of VGAM137 host target RNA, herein designated VGAM HOST TARGET RNA. This complementary binding is due to the fact that the nucleotide sequence of VGAM137 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM137 RNA, herein designated VGAM RNA, may have a different number of host target binding sites in untranslated regions of a VGAM137 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM137 RNA, herein designated VGAM RNA, to host target binding sites on VGAM137 host target RNA, herein designated VGAM HOST TARGET RNA, such as BINDING SITE I, BINDING SITE II and BINDING SITE III, inhibits translation of VGAM137 host target RNA into VGAM137 host target protein, herein designated VGAM HOST TARGET PROTEIN. VGAM host target protein is therefore outlined by a broken line.

It is appreciated that VGAM137 host target gene, herein designated VGAM HOST TARGET GENE, in fact represents a plurality of VGAM137 host target genes. The mRNA of each one of this plurality of VGAM137 host target genes comprises one or more host target binding sites, each having a nucleotide sequence which is at least partly complementary to VGAM137 RNA, herein designated VGAM RNA, and which when bound by VGAM137 RNA causes inhibition of translation of respective one or more VGAM137 host target proteins.

It is further appreciated by one skilled in the art that the mode of translational inhibition illustrated by FIG. 1 with specific reference to translational inhibition exerted by VGAM137 gene, herein designated VGAM GENE, on one or more VGAM137 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM137 is inhibition of expression of host target genes, as part of a novel viral mechanism of attacking a host. Accordingly, utilities of VGAM137 include diagnosis, prevention and treatment of viral infection by Vaccinia Virus. Specific functions, and accordingly utilities, of VGAM137 correlate with, and may be deduced from, the identity of the host target genes which VGAM137 binds and inhibits, and the function of these host target genes, as elaborated hereinbelow.

Nucleotide sequences of the VGAM137 precursor RNA, herein designated VGAM PRECURSOR RNA, and of the ‘diced’ VGAM137 RNA, herein designated VGAM RNA, and a schematic representation of the secondary folding of VGAM137 folded precursor RNA, herein designated VGAM FOLDED PRECURSOR RNA, of VGAM137 are further described hereinbelow with reference to Table 1.

Nucleotide sequences of host target binding sites, such as BINDING SITE-I, BINDING SITE-II and BINDING SITE-III of FIG. 1, found on VGAM137 host target RNA, and schematic representation of the complementarity of each of these host target binding sites to VGAM137 RNA, herein designated VGAM RNA, are described hereinbelow with reference to Table 2.

As mentioned hereinabove with reference to FIG. 1, a function of VGAM137 gene, herein designated VGAM is inhibition of expression of VGAM137 target genes. It is appreciated that specific functions, and accordingly utilities, of VGAM137 correlate with, and may be deduced from, the identity of the target genes which VGAM137 binds and inhibits, and the function of these target genes, as elaborated hereinbelow.

Apical Protein-like (Xenopus laevis) (APXL, Accession NM_{—}001649) is a VGAM137 host target gene. APXL BINDING SITE is HOST TARGET binding site found in the 3′ untranslated region of mRNA encoded by APXL, corresponding to a HOST TARGET binding site such as BINDING SITE I, BINDING SITE II or BINDING SITE III. Table 2 illustrates the complementarity of the nucleotide sequences of APXL BINDING SITE, designated SEQ ID:842, to the nucleotide sequence of VGAM137 RNA, herein designated VGAM RNA, also designated SEQ ID:472.

A function of VGAM137 is therefore inhibition of Apical Protein-like (Xenopus laevis) (APXL, Accession NM_{—}001649), a gene which is implicated in amiloride-sensitive sodium channel activity. Accordingly, utilities of VGAM137 include diagnosis, prevention and treatment of diseases and clinical conditions associated with APXL. The function of APXL has been established by previous studies. APXL is a human homolog of the Xenopus laevis APX gene which is implicated in amiloride-sensitive sodium channel activity (Schiaffino et al., 1995). The gene contains 10 exons and spans approximately 160 kb of Xp22.3 in the ocular albinism type 1 (OA1; 300500) critical region. The full-length mRNA is approximately 7.5 kb, and Schiaffino et al. (1995) isolated several clones from a retinal cDNA library that corresponded to this mRNA. The authors found that, along with retina, the gene is expressed in melanoma cells, brain, placenta, lung, kidney, and pancreas. The protein is 1,616 amino acids in length. APXL was deleted in 2 patients with contiguous gene syndromes including OA1 and in 1 patient with isolated OA1. Comparative mapping of the X chromosome in eutherian mammals has revealed distinct regions of conservation as well as evolutionary rearrangements between human and mouse. Dinulos et al. (1996) mapped the murine homologs of OA1 and APXL. They found that the 2 genes map to bands F2-F3 in both M. spretus and the laboratory strains C57BL/6J, defining a new rearrangement between human and mouse X chromosomes.

Full details of the abovementioned studies are described in the following publications, the disclosure of which are hereby incorporated by reference:
Dinulos, M. B.; Bassi, M. T.; Rugarli, E. I.; Chapman, V.; Ballabio, A.; Disteche, C. M.: A new region of conservation is defined between human and mouse X chromosomes. Genomics 35: 244-247, 1996.; and
Schiaffino, M. V.; Bassi, M. T.; Rugarli, E. I.; Renieri, A.; Galli, L.; Ballabio, A.: Cloning of a human homologue of the Xenopus laevis APX gene from the ocular albinism type 1 criti.

Further studies establishing the function and utilities of APXL are found in John Hopkins OMIM database record ID 300103, and in sited publications numbered 1990-1991 listed in the bibliography section hereinbelow, which are also hereby incorporated by reference. Cockayne Syndrome 1 (classical) (CKN1, Accession NM_{—}000082) is another VGAM137 host target gene. CKN1 BINDING SITE is HOST TARGET binding site found in the 3′ untranslated region of mRNA encoded by CKN1, corresponding to a HOST TARGET binding site such as BINDING SITE I, BINDING SITE II or BINDING SITE III. Table 2 illustrates the complementarity of the nucleotide sequences of CKN1 BINDING SITE, designated SEQ ID:702, to the nucleotide sequence of VGAM137 RNA, herein designated VGAM RNA, also designated SEQ ID:472.

Another function of VGAM137 is therefore inhibition of Cockayne Syndrome 1 (classical) (CKN1, Accession NM_{—}000082). Accordingly, utilities of VGAM137 include diagnosis, prevention and treatment of diseases and clinical conditions associated with CKN1. High Mobility Group AT-hook 2 (HMGA2, Accession NM_{—}003483) is another VGAM137 host target gene. HMGA2 BINDING SITE is HOST TARGET binding site found in the 3′ untranslated region of mRNA encoded by HMGA2, corresponding to a HOST TARGET binding site such as BINDING SITE I, BINDING SITE II or BINDING SITE III. Table 2 illustrates the complementarity of the nucleotide sequences of HMGA2 BINDING SITE, designated SEQ ID: 1032, to the nucleotide sequence of VGAM137 RNA, herein designated VGAM RNA, also designated SEQ ID:472.

Another function of VGAM137 is therefore inhibition of High Mobility Group AT-hook 2 (HMGA2, Accession NM_{—}003483), a gene which may affect transcription and cell differentiation; shares common DNA-binding motif with other HMG HMG I/Y family members. Accordingly, utilities of VGAM137 include diagnosis, prevention and treatment of diseases and clinical conditions associated with HMGA2. The function of HMGA2 has been established by previous studies. Ashar et al. (1995) considered the HMGIC gene a good candidate for involvement in lipoma for several reasons, including the facts that it encodes a transcriptional regulating factor, that the pygmy mouse had disproportionately less fat than normal litter mates, and that the mouse gene maps to a region of chromosome 10 with homology of synteny to human 12q14-q15. Therefore, they cloned the human gene and investigated its possible role in lipomas. In FISH studies, Ashar et al. (1995) found apparent deletion of the 3-prime end of the HMGIC gene in translocations associated with lipoma. Chimeric transcripts were isolated from 2 lipomas in which HMGIC DNA-binding domains (AT hook motifs) were fused to either a LIM or an acidic transactivator domain. The identification of a gene rearranged in a benign neoplastic process suggests a role for HMGIC in adipogenesis and mesenchymal differentiation. Animal model experiments lend further support to the function of HMGA2. To evaluate the role of the HMGIC component in the development of lipoma, Arlotta et al. (2000) expressed the 3 DNA-binding domains of HMGIC in transgenic mice. Despite the ubiquitous expression of the truncated HMGIC protein, the transgenic mice developed a selective abundance of fat tissue early in life, showed marked adipose tissue inflammation, and had an abnormally high incidence of lipomas. These findings demonstrated that the DNA-binding domain of HMGIC, in the absence of a C-terminal fusion partner, are sufficient to perturb adipogenesis and predispose to lipomas.

It is appreciated that the abovementioned animal model for HMGA2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ashar, H. R.; Schoenberg Fejzo, M.; Tkachenko, A.; Zhou, X.; Fletcher, J. A.; Weremowicz, S.; Morton, C. C.; Chada, K.: Disruption of the architectural factor HMGI-C: DNA-binding AT hook motifs fused in lipomas to distinct transcriptional regulatory domains. Cell 82: 57-65, 1995.; and
Arlotta, P.; Tai, A. K.-F.; Manfioletti, G.; Clifford, C.; Jay, G.; Ono, S. J.: Transgenic mice expressing a truncated form of the high mobility group I—C protein develop adiposity and.

Further studies establishing the function and utilities of HMGA2 are found in John Hopkins OMIM database record ID 600698, and in sited publications numbered 2306-2318, 2345, 2370-2371, 79 and 2372 listed in the bibliography section hereinbelow, which are also hereby incorporated by reference. Zinc Finger Protein 36 (KOX 18) (ZNF36, Accession XM_{—}168302) is another VGAM137 host target gene. ZNF36 BINDING SITE is HOST TARGET binding site found in the 3′ untranslated region of mRNA encoded by ZNF36, corresponding to a HOST TARGET binding site such as BINDING SITE I, BINDING SITE II or BINDING SITE III. Table 2 illustrates the complementarity of the nucleotide sequences of ZNF36 BINDING SITE, designated SEQ ID:3626, to the nucleotide sequence of VGAM137 RNA, herein designated VGAM RNA, also designated SEQ ID:472.

Another function of VGAM137 is therefore inhibition of Zinc Finger Protein 36 (KOX 18) (ZNF36, Accession XM_{—}168302), a gene which may be involved in transcriptional regulation. Accordingly, utilities of VGAM137 include diagnosis, prevention and treatment of diseases and clinical conditions associated with ZNF36. The function of ZNF36 has been established by previous studies. By screening a human insulinoma cDNA library with a degenerate oligonucleotide corresponding to the H/C linker sequence, Tommerup et al. (1993) isolated cDNAs potentially encoding zinc finger proteins. Tommerup and Vissing (1995) performed sequence analysis on a number of these cDNAs and identified several novel zinc finger protein genes, including ZNF36, which they called ZNF139. The ZNF139 cDNA predicts a protein belonging to the Kruppel family of zinc finger proteins. By isotopic in situ hybridization, Rousseau-Merck et al. (1995) mapped the ZNF36 gene, which they called KOX18, to 7q21-q22. From pulsed field gel electrophoresis studies, they showed that KOX18 is within less than 250 kb of KOX25 (ZNF38; 601261). Rousseau-Merck et al. (1995) tabulated 18 different KOX genes that had been located in pairs within 9 DNA fragments of 200 to 580 kb on 7 different chromosomes. By FISH, Tommerup and Vissing (1995) mapped the ZNF36 gene to 7q21.3-q22.1.

Full details of the abovementioned studies are described in the following publications, the disclosure of which are hereby incorporated by reference:
Tommerup, N.; Vissing, H.: Isolation and fine mapping of 16 novel human zinc finger-encoding cDNAs identify putative candidate genes for developmental and malignant disorders. Genomics 27: 259-264, 1995.; and
Rousseau-Merck, M.-F.; Duro, D.; Berger, R.; Thiesen, H. J.: Chromosomal localization of two KOX zinc finger genes on chromosome bands 7q21-q22. Ann. Genet. 38: 81-84, 1995.

Further studies establishing the function and utilities of ZNF36 are found in John Hopkins OMIM database record ID 601260, and in sited publications numbered 208 and 2082 listed in the bibliography section hereinbelow, which are also hereby incorporated by reference. FLJ23191 (Accession NM_{—}024574) is another VGAM137 host target gene. FLJ23191 BINDING SITE is HOST TARGET binding site found in the 3′ untranslated region of mRNA encoded by FLJ23191, corresponding to a HOST TARGET binding site such as BINDING SITE I, BINDING SITE II or BINDING SITE III. Table 2 illustrates the complementarity of the nucleotide sequences of FLJ23191 BINDING SITE, designated SEQ ID:2065, to the nucleotide sequence of VGAM137 RNA, herein designated VGAM RNA, also designated SEQ ID:472.

Another function of VGAM137 is therefore inhibition of FLJ23191 (Accession NM_{—}024574). Accordingly, utilities of VGAM137 include diagnosis, prevention and treatment of diseases and clinical conditions associated with FLJ23191. KIAA0841 (Accession XM_{—}049237) is another VGAM137 host target gene. KIAA0841 BINDING SITE is HOST TARGET binding site found in the 3′ untranslated region of mRNA encoded by KIAA0841, corresponding to a HOST TARGET binding site such as BINDING SITE I, BINDING SITE II or BINDING SITE III. Table 2 illustrates the complementarity of the nucleotide sequences of KIAA0841 BINDING SITE, designated SEQ ID:2918, to the nucleotide sequence of VGAM137 RNA, herein designated VGAM RNA, also designated SEQ ID:472.

Another function of VGAM137 is therefore inhibition of KIAA0841 (Accession XM_{—}049237). Accordingly, utilities of VGAM137 include diagnosis, prevention and treatment of diseases and clinical conditions associated with KIAA0841. KIAA1243 (Accession XM_{—}057057) is another VGAM137 host target gene. KIAA1243 BINDING SITE is HOST TARGET binding site found in the 3′ untranslated region of mRNA encoded by KIAA1243, corresponding to a HOST TARGET binding site such as BINDING SITE I, BINDING SITE II or BINDING SITE III. Table 2 illustrates the complementarity of the nucleotide sequences of KIAA1243 BINDING SITE, designated SEQ ID:2977, to the nucleotide sequence of VGAM137 RNA, herein designated VGAM RNA, also designated SEQ ID:472.

Another function of VGAM137 is therefore inhibition of KIAA1243 (Accession XM_{—}057057). Accordingly, utilities of VGAM137 include diagnosis, prevention and treatment of diseases and clinical conditions associated with KIAA1243. Ring Finger Protein 20 (RNF20, Accession NM_{—}019592) is another VGAM137 host target gene. RNF20 BINDING SITE is HOST TARGET binding site found in the 3′ untranslated region of mRNA encoded by RNF20, corresponding to a HOST TARGET binding site such as BINDING SITE I, BINDING SITE II or BINDING SITE III. Table 2 illustrates the complementarity of the nucleotide sequences of RNF20 BINDING SITE, designated SEQ ID:1882, to the nucleotide sequence of VGAM137 RNA, herein designated VGAM RNA, also designated SEQ ID:472.

Another function of VGAM137 is therefore inhibition of Ring Finger Protein 20 (RNF20, Accession NM_{—}019592). Accordingly, utilities of VGAM137 include diagnosis, prevention and treatment of diseases and clinical conditions associated with RNF20. LOC118851 (Accession XM_{—}061180) is another VGAM137 host target gene. LOC118851 BINDING SITE is HOST TARGET binding site found in the 3′ untranslated region of mRNA encoded by LOC118851, corresponding to a HOST TARGET binding site such as BINDING SITE I, BINDING SITE II or BINDING SITE III. Table 2 illustrates the complementarity of the nucleotide sequences of LOC118851 BINDING SITE, designated SEQ ID:3025, to the nucleotide sequence of VGAM137 RNA, herein designated VGAM RNA, also designated SEQ ID:472.

Another function of VGAM137 is therefore inhibition of LOC118851 (Accession XM_{—}061180). Accordingly, utilities of VGAM137 include diagnosis, prevention and treatment of diseases and clinical conditions associated with LOC118851. LOC150005 (Accession XM_{—}097795) is another VGAM137 host target gene. LOC150005 BINDING SITE is HOST TARGET binding site found in the 3′ untranslated region of mRNA encoded by LOC150005, corresponding to a HOST TARGET binding site such as BINDING SITE I, BINDING SITE II or BINDING SITE III. Table 2 illustrates the complementarity of the nucleotide sequences of LOC150005 BINDING SITE, designated SEQ ID:3322, to the nucleotide sequence of VGAM137 RNA, herein designated VGAM RNA, also designated SEQ ID:472.

Another function of VGAM137 is therefore inhibition of LOC150005 (Accession XM_{—}097795). Accordingly, utilities of VGAM137 include diagnosis, prevention and treatment of diseases and clinical conditions associated with LOC150005. LOC221036 (Accession XM_{—}167403) is another VGAM137 host target gene. LOC221036 BINDING SITE is HOST TARGET binding site found in the 3′ untranslated region of mRNA encoded by LOC221036, corresponding to a HOST TARGET binding site such as BINDING SITE I, BINDING SITE II or BINDING SITE III. Table 2 illustrates the complementarity of the nucleotide sequences of LOC221036 BINDING SITE, designated SEQ ID:3585, to the nucleotide sequence of VGAM137 RNA, herein designated VGAM RNA, also designated SEQ ID:472.

Another function of VGAM137 is therefore inhibition of LOC221036 (Accession XM_{—}167403). Accordingly, utilities of VGAM137 include diagnosis, prevention and treatment of diseases and clinical conditions associated with LOC221036. FIG. 1 further provides a conceptual description of a novel bioinformatically detected viral gene of the present invention, referred to here as Viral Genomic Address Messenger 138 (VGAM138) viral gene, which modulates expression of respective host target genes thereof, the function and utility of which host target genes is known in the art.

VGAM138 is a novel bioinformatically detected regulatory, non protein coding, viral micro RNA (miRNA) gene. The method by which VGAM138 was detected is described hereinabove with reference to FIGS. 1-8.

VGAM138 gene, herein designated VGAM GENE, is a viral gene contained in the genome of Vaccinia Virus. VGAM138 host target gene, herein designated VGAM HOST TARGET GENE, is a human gene contained in the human genome.

VGAM138 gene encodes a VGAM138 precursor RNA, herein designated VGAM PRECURSOR RNA. Similar to other miRNA genes, and unlike most ordinary genes, VGAM138 precursor RNA does not encode a protein. A nucleotide sequence identical or highly similar to the nucleotide sequence of VGAM138 precursor RNA is designated SEQ ID:124, and is provided hereinbelow with reference to the sequence listing part. Nucleotide sequence SEQ ID:124 is located at position 63387 relative to the genome of Vaccinia Virus.

VGAM138 precursor RNA folds onto itself, forming VGAM138 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM138 folded precursor RNA into VGAM138 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM138 RNA is designated SEQ ID:473, and is provided hereinbelow with reference to the sequence listing part.

VGAM138 host target gene, herein designated VGAM HOST TARGET GENE, encodes a corresponding messenger RNA, VGAM138 host target RNA, herein designated VGAM HOST TARGET RNA. VGAM138 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM138 RNA, herein designated VGAM RNA, binds complementarily to one or more host target binding sites located in untranslated regions of VGAM138 host target RNA, herein designated VGAM HOST TARGET RNA. This complementary binding is due to the fact that the nucleotide sequence of VGAM138 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM138 RNA, herein designated VGAM RNA, may have a different number of host target binding sites in untranslated regions of a VGAM138 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM138 RNA, herein designated VGAM RNA, to host target binding sites on VGAM138 host target RNA, herein designated VGAM HOST TARGET RNA, such as BINDING SITE I, BINDING SITE II and BINDING SITE III, inhibits translation of VGAM138 host target RNA into VGAM138 host target protein, herein designated VGAM HOST TARGET PROTEIN. VGAM host target protein is therefore outlined by a broken line.

It is appreciated that VGAM138 host target gene, herein designated VGAM HOST TARGET GENE, in fact represents a plurality of VGAM138 host target genes. The mRNA of each one of this plurality of VGAM138 host target genes comprises one or more host target binding sites, each having a nucleotide sequence which is at least partly complementary to VGAM138 RNA, herein designated VGAM RNA, and which when bound by VGAM138 RNA causes inhibition of translation of respective one or more VGAM138 host target proteins.

It is further appreciated by one skilled in the art that the mode of translational inhibition illustrated by FIG. 1 with specific reference to translational inhibition exerted by VGAM138 gene, herein designated VGAM GENE, on one or more VGAM138 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM138 is inhibition of expression of host target genes, as part of a novel viral mechanism of attacking a host. Accordingly, utilities of VGAM138 include diagnosis, prevention and treatment of viral infection by Vaccinia Virus. Specific functions, and accordingly utilities, of VGAM138 correlate with, and may be deduced from, the identity of the host target genes which VGAM138 binds and inhibits, and the function of these host target genes, as elaborated hereinbelow.

Nucleotide sequences of the VGAM138 precursor RNA, herein designated VGAM PRECURSOR RNA, and of the ‘diced’ VGAM138 RNA, herein designated VGAM RNA, and a schematic representation of the secondary folding of VGAM138 folded precursor RNA, herein designated VGAM FOLDED PRECURSOR RNA, of VGAM138 are further described hereinbelow with reference to Table 1.

Nucleotide sequences of host target binding sites, such as BINDING SITE-I, BINDING SITE-II and BINDING SITE-III of FIG. 1, found on VGAM138 host target RNA, and schematic representation of the complementarity of each of these host target binding sites to VGAM138 RNA, herein designated VGAM RNA, are described hereinbelow with reference to Table 2.

As mentioned hereinabove with reference to FIG. 1, a function of VGAM138 gene, herein designated VGAM is inhibition of expression of VGAM138 target genes. It is appreciated that specific functions, and accordingly utilities, of VGAM138 correlate with, and may be deduced from, the identity of the target genes which VGAM138 binds and inhibits, and the function of these target genes, as elaborated hereinbelow.

Component of Oligomeric Golgi Complex 7 (COG7, Accession XM_{—}041725) is a VGAM138 host target gene. COG7 BINDING SITE is HOST TARGET binding site found in the 3′ untranslated region of mRNA encoded by COG7, corresponding to a HOST TARGET binding site such as BINDING SITE I, BINDING SITE II or BINDING SITE III. Table 2 illustrates the complementarity of the nucleotide sequences of COG7 BINDING SITE, designated SEQ ID:2795, to the nucleotide sequence of VGAM138 RNA, herein designated VGAM RNA, also designated SEQ ID:473.

A function of VGAM138 is therefore inhibition of Component of Oligomeric Golgi Complex 7 (COG7, Accession XM_{—}041725). Accordingly, utilities of VGAM138 include diagnosis, prevention and treatment of diseases and clinical conditions associated with COG7. Dual Specificity Phosphatase 5 (DUSP5, Accession NM_{—}004419) is another VGAM138 host target gene. DUSP5 BINDING SITE is HOST TARGET binding site found in the 3′ untranslated region of mRNA encoded by DUSP5, corresponding to a HOST TARGET binding site such as BINDING SITE I, BINDING SITE II or BINDING SITE III. Table 2 illustrates the complementarity of the nucleotide sequences of DUSP5 BINDING SITE, designated SEQ ID:1111, to the nucleotide sequence of VGAM138 RNA, herein designated VGAM RNA, also designated SEQ ID:473.

Another function of VGAM138 is therefore inhibition of Dual Specificity Phosphatase 5 (DUSP5, Accession NM_{—}004419), a gene which displays phosphatase activity toward several substrates. Accordingly, utilities of VGAM138 include diagnosis, prevention and treatment of diseases and clinical conditions associated with DUSP5. The function of DUSP5 has been established by previous studies. The VH1 phosphatase encoded by vaccinia virus is a dual-specificity protein-tyrosine phosphatase (OMIM Ref. No. PTPase) which hydrolyzes substrates phosphorylated on both tyrosine and serine/threonine residues. VH1-like PTPases have been identified in humans and other organisms. See DUSP1 (OMIM Ref. No. 600714). To identify additional human dual-specificity PTPases, Martell et al. (1994) screened a genomic library with a partial DUSP1 cDNA. They isolated several novel PTPase genes, including 1 which they designated HVH3 for human VH1-like PTPase-3. Kwak and Dixon (1995) cloned human placental HVH3 cDNAs and reported that the predicted protein has 384 amino acids. Using immunofluorescence, they determined that epitope-tagged HVH3 is localized primarily in the nucleus of mammalian cells. Ishibashi et al. (1994) isolated HVH3 cDNAs from a human mammary epithelial cell cDNA library and found that the predicted protein has 397 amino acids. In vitro, recombinant protein containing the catalytic domain of HVH3 displayed phosphatase activity toward several substrates. The highest relative activity was toward ERK1 (OMIM Ref. No. 601795), suggesting that it may be a target for HVH3 activity in vivo. Northern blot analysis revealed that HVH3 is expressed as a 2.5-kb mRNA in a variety of tissues, with the highest levels in pancreas and brain. HVH3 expression was induced by serum stimulation of fibroblasts and by heat shock, with similar kinetics to those observed with DUSP1. As has been proposed for other dual-specificity PTPases like DUSP1 and DUSP2 (OMIM Ref. No. 603068), Ishibashi et al. (1994) suggested that the induction of HVH3 may lead to the deactivation of mitogen- or stress-activated protein kinases, thereby restoring these signaling pathways to their mitogen- or stress-sensitive state. By fluorescence in situ hybridization, Martell et al. (1994) mapped the HVH3 gene to 10q25

Full details of the abovementioned studies are described in the following publications, the disclosure of which are hereby incorporated by reference:
Ishibashi, T.; Bottaro, D. P.; Michieli, P.; Kelley, C. A.; Aaronson, S. A.: A novel dual specificity phosphatase induced by serum stimulation and heat shock. J. Biol. Chem. 269: 29897-29902, 1994.; and
Martell, K. J.; Kwak, S.; Hakes, D. J.; Dixon, J. E.; Trent, J. M.: Chromosomal localization of four human VH1-like protein-tyrosine phosphatases. Genomics 22: 462-464, 1994.

Further studies establishing the function and utilities of DUSP5 are found in John Hopkins OMIM database record ID 603069, and in sited publications numbered 2000-200 and 2280 listed in the bibliography section hereinbelow, which are also hereby incorporated by reference. Chromosome 6 Open Reading Frame 29 (C6orf29, Accession NM_{—}032794) is another VGAM138 host target gene. C6orf29 BINDING SITE is HOST TARGET binding site found in the 3′ untranslated region of mRNA encoded by C6orf29, corresponding to a HOST TARGET binding site such as BINDING SITE I, BINDING SITE II or BINDING SITE III. Table 2 illustrates the complementarity of the nucleotide sequences of C6orf29 BINDING SITE, designated SEQ ID:2278, to the nucleotide sequence of VGAM138 RNA, herein designated VGAM RNA, also designated SEQ ID:473.

Another function of VGAM138 is therefore inhibition of Chromosome 6 Open Reading Frame 29 (C6orf29, Accession NM_{—}032794). Accordingly, utilities of VGAM138 include diagnosis, prevention and treatment of diseases and clinical conditions associated with C6orf29. CGR19 (Accession NM_{—}006568) is another VGAM138 host target gene. CGR19 BINDING SITE is HOST TARGET binding site found in the 3′ untranslated region of mRNA encoded by CGR19, corresponding to a HOST TARGET binding site such as BINDING SITE I, BINDING SITE II or BINDING SITE III. Table 2 illustrates the complementarity of the nucleotide sequences of CGR19 BINDING SITE, designated SEQ ID:1311, to the nucleotide sequence of VGAM138 RNA, herein designated VGAM RNA, also designated SEQ ID:473.

Another function of VGAM138 is therefore inhibition of CGR19 (Accession NM_{—}006568). Accordingly, utilities of VGAM138 include diagnosis, prevention and treatment of diseases and clinical conditions associated with CGR19. Down Syndrome Critical Region Gene 6 (DSCR6, Accession NM_{—}018962) is another VGAM138 host target gene. DSCR6 BINDING SITE is HOST TARGET binding site found in the 3′ untranslated region of mRNA encoded by DSCR6, corresponding to a HOST TARGET binding site such as BINDING SITE I, BINDING SITE II or BINDING SITE III. Table 2 illustrates the complementarity of the nucleotide sequences of DSCR6 BINDING SITE, designated SEQ ID:1865, to the nucleotide sequence of VGAM138 RNA, herein designated VGAM RNA, also designated SEQ ID:473.

Another function of VGAM138 is therefore inhibition of Down Syndrome Critical Region Gene 6 (DSCR6, Accession NM_{—}018962). Accordingly, utilities of VGAM138 include diagnosis, prevention and treatment of diseases and clinical conditions associated with DSCR6. ETL (Accession NM_{—}022159) is another VGAM138 host target gene. ETL BINDING SITE is HOST TARGET binding site found in the 3′ untranslated region of mRNA encoded by ETL, corresponding to a HOST TARGET binding site such as BINDING SITE I, BINDING SITE II or BINDING SITE III. Table 2 illustrates the complementarity of the nucleotide sequences of ETL BINDING SITE, designated SEQ ID:1982, to the nucleotide sequence of VGAM138 RNA, herein designated VGAM RNA, also designated SEQ ID:473.

Another function of VGAM138 is therefore inhibition of ETL (Accession NM_{—}022159). Accordingly, utilities of VGAM138 include diagnosis, prevention and treatment of diseases and clinical conditions associated with ETL. FLJ21269 (Accession NM_{—}025107) is another VGAM138 host target gene. FLJ21269 BINDING SITE is HOST TARGET binding site found in the 3′ untranslated region of mRNA encoded by FLJ21269, corresponding to a HOST TARGET binding site such as BINDING SITE I, BINDING SITE II or BINDING SITE III. Table 2 illustrates the complementarity of the nucleotide sequences of FLJ21269 BINDING SITE, designated SEQ ID:2138, to the nucleotide sequence of VGAM138 RNA, herein designated VGAM RNA, also designated SEQ ID:473.

Another function of VGAM138 is therefore inhibition of FLJ21269 (Accession NM_{—}025107). Accordingly, utilities of VGAM138 include diagnosis, prevention and treatment of diseases and clinical conditions associated with FLJ21269. HNT (Accession NM_{—}016522) is another VGAM138 host target gene. HNT BINDING SITE is HOST TARGET binding site found in the 3′ untranslated region of mRNA encoded by HNT, corresponding to a HOST TARGET binding site such as BINDING SITE I, BINDING SITE II or BINDING SITE III. Table 2 illustrates the complementarity of the nucleotide sequences of HNT BINDING SITE, designated SEQ ID: 1692, to the nucleotide sequence of VGAM138 RNA, herein designated VGAM RNA, also designated SEQ ID:473.

Another function of VGAM138 is therefore inhibition of HNT (Accession NM_{—}016522). Accordingly, utilities of VGAM138 include diagnosis, prevention and treatment of diseases and clinical conditions associated with HNT. Potassium Voltage-gated Channel, Shab-related Sub family, Member 2 (KCNB2, Accession XM_{—}171186) is another VGAM138 host target gene. KCNB2 BINDING SITE is HOST TARGET binding site found in the 3′ untranslated region of mRNA encoded by KCNB2, corresponding to a HOST TARGET binding site such as BINDING SITE I, BINDING SITE II or BINDING SITE III. Table 2 illustrates the complementarity of the nucleotide sequences of KCNB2 BINDING SITE, designated SEQ ID:3690, to the nucleotide sequence of VGAM138 RNA, herein designated VGAM RNA, also designated SEQ ID:473.

Another function of VGAM138 is therefore inhibition of Potassium Voltage-gated Channel, Shab-related Sub family, Member 2 (KCNB2, Accession XM_{—}171186). Accordingly, utilities of VGAM138 include diagnosis, prevention and treatment of diseases and clinical conditions associated with KCNB2. KIAA0794 (Accession XM_{—}087353) is another VGAM138 host target gene. KIAA0794 BINDING SITE is HOST TARGET binding site found in the 3′ untranslated region of mRNA encoded by KIAA0794, corresponding to a HOST TARGET binding site such as BINDING SITE I, BINDING SITE II or BINDING SITE III. Table 2 illustrates the complementarity of the nucleotide sequences of KIAA0794 BINDING SITE, designated SEQ ID:3163, to the nucleotide sequence of VGAM138 RNA, herein designated VGAM RNA, also designated SEQ ID:473.

Another function of VGAM138 is therefore inhibition of KIAA0794 (Accession XM_{—}087353). Accordingly, utilities of VGAM138 include diagnosis, prevention and treatment of diseases and clinical conditions associated with KIAA0794. LOC123342 (Accession XM_{—}063602) is another VGAM138 host target gene. LOC123342 BINDING SITE is HOST TARGET binding site found in the 5′ untranslated region of mRNA encoded by LOC123342, corresponding to a HOST TARGET binding site such as BINDING SITE I, BINDING SITE II or BINDING SITE III. Table 2 illustrates the complementarity of the nucleotide sequences of LOC123342 BINDING SITE, designated SEQ ID:3031, to the nucleotide sequence of VGAM138 RNA, herein designated VGAM RNA, also designated SEQ ID:473.

Another function of VGAM138 is therefore inhibition of LOC123342 (Accession XM_{—}063602). Accordingly, utilities of VGAM138 include diagnosis, prevention and treatment of diseases and clinical conditions associated with LOC123342. LOC144920 (Accession XM_{—}096688) is another VGAM138 host target gene. LOC144920 BINDING SITE is HOST TARGET binding site found in the 3′ untranslated region of mRNA encoded by LOC144920, corresponding to a HOST TARGET binding site such as BINDING SITE I, BINDING SITE II or BINDING SITE III. Table 2 illustrates the complementarity of the nucleotide sequences of LOC144920 BINDING SITE, designated SEQ ID:3270, to the nucleotide sequence of VGAM138 RNA, herein designated VGAM RNA, also designated SEQ ID:473.

Another function of VGAM138 is therefore inhibition of LOC144920 (Accession XM_{—}096688). Accordingly, utilities of VGAM138 include diagnosis, prevention and treatment of diseases and clinical conditions associated with LOC144920. LOC153688 (Accession XM_{—}098416) is another VGAM138 host target gene. LOC153688 BINDING SITE is HOST TARGET binding site found in the 3′ untranslated region of mRNA encoded by LOC153688, corresponding to a HOST TARGET binding site such as BINDING SITE I, BINDING SITE II or BINDING SITE III. Table 2 illustrates the complementarity of the nucleotide sequences of LOC153688 BINDING SITE, designated SEQ ID:3370, to the nucleotide sequence of VGAM138 RNA, herein designated VGAM RNA, also designated SEQ ID:473.

Another function of VGAM138 is therefore inhibition of LOC153688 (Accession XM_{—}098416). Accordingly, utilities of VGAM138 include diagnosis, prevention and treatment of diseases and clinical conditions associated with LOC153688. LOC51336 (Accession NM_{—}016646) is another VGAM138 host target gene. LOC51336 BINDING SITE is HOST TARGET binding site found in the 5′ untranslated region of mRNA encoded by LOC51336, corresponding to a HOST TARGET binding site such as BINDING SITE I, BINDING SITE II or BINDING SITE III. Table 2 illustrates the complementarity of the nucleotide sequences of LOC51336 BINDING SITE, designated SEQ ID:1707, to the nucleotide sequence of VGAM138 RNA, herein designated VGAM RNA, also designated SEQ ID:473.

Another function of VGAM138 is therefore inhibition of LOC51336 (Accession NM_{—}016646). Accordingly, utilities of VGAM138 include diagnosis, prevention and treatment of diseases and clinical conditions associated with LOC51336. FIG. 1 further provides a conceptual description of a novel bioinformatically detected viral gene of the present invention, referred to here as Viral Genomic Address Messenger 139 (VGAM139) viral gene, which modulates expression of respective host target genes thereof, the function and utility of which host target genes is known in the art.

VGAM139 is a novel bioinformatically detected regulatory, non protein coding, viral micro RNA (miRNA) gene. The method by which VGAM139 was detected is described hereinabove with reference to FIGS. 1-8.

VGAM139 gene, herein designated VGAM GENE, is a viral gene contained in the genome of Vaccinia Virus. VGAM139 host target gene, herein designated VGAM HOST TARGET GENE, is a human gene contained in the human genome.

VGAM139 gene encodes a VGAM139 precursor RNA, herein designated VGAM PRECURSOR RNA. Similar to other miRNA genes, and unlike most ordinary genes, VGAM139 precursor RNA does not encode a protein. A nucleotide sequence identical or highly similar to the nucleotide sequence of VGAM139 precursor RNA is designated SEQ ID:125, and is provided hereinbelow with reference to the sequence listing part. Nucleotide sequence SEQ ID:125 is located at position 63571 relative to the genome of Vaccinia Virus.

VGAM139 precursor RNA folds onto itself, forming VGAM139 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM139 folded precursor RNA into VGAM139 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM139 RNA is designated SEQ ID:474, and is provided hereinbelow with reference to the sequence listing part.

VGAM139 host target gene, herein designated VGAM HOST TARGET GENE, encodes a corresponding messenger RNA, VGAM139 host target RNA, herein designated VGAM HOST TARGET RNA. VGAM139 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM139 RNA, herein designated VGAM RNA, binds complementarily to one or more host target binding sites located in untranslated regions of VGAM139 host target RNA, herein designated VGAM HOST TARGET RNA. This complementary binding is due to the fact that the nucleotide sequence of VGAM139 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM139 RNA, herein designated VGAM RNA, may have a different number of host target binding sites in untranslated regions of a VGAM139 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM139 RNA, herein designated VGAM RNA, to host target binding sites on VGAM139 host target RNA, herein designated VGAM HOST TARGET RNA, such as BINDING SITE I, BINDING SITE II and BINDING SITE III, inhibits translation of VGAM139 host target RNA into VGAM139 host target protein, herein designated VGAM HOST TARGET PROTEIN. VGAM host target protein is therefore outlined by a broken line.

It is appreciated that VGAM139 host target gene, herein designated VGAM HOST TARGET GENE, in fact represents a plurality of VGAM139 host target genes. The mRNA of each one of this plurality of VGAM139 host target genes comprises one or more host target binding sites, each having a nucleotide sequence which is at least partly complementary to VGAM139 RNA, herein designated VGAM RNA, and which when bound by VGAM139 RNA causes inhibition of translation of respective one or more VGAM139 host target proteins.

It is further appreciated by one skilled in the art that the mode of translational inhibition illustrated by FIG. 1 with specific reference to translational inhibition exerted by VGAM139 gene, herein designated VGAM GENE, on one or more VGAM139 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM139 is inhibition of expression of host target genes, as part of a novel viral mechanism of attacking a host. Accordingly, utilities of VGAM139 include diagnosis, prevention and treatment of viral infection by Vaccinia Virus. Specific functions, and accordingly utilities, of VGAM139 correlate with, and may be deduced from, the identity of the host target genes which VGAM139 binds and inhibits, and the function of these host target genes, as elaborated hereinbelow.

Nucleotide sequences of the VGAM139 precursor RNA, herein designated VGAM PRECURSOR RNA, and of the ‘diced’ VGAM139 RNA, herein designated VGAM RNA, and a schematic representation of the secondary folding of VGAM139 folded precursor RNA, herein designated VGAM FOLDED PRECURSOR RNA, of VGAM139 are further described hereinbelow with reference to Table 1.

Nucleotide sequences of host target binding sites, such as BINDING SITE-I, BINDING SITE-II and BINDING SITE-III of FIG. 1, found on VGAM139 host target RNA, and schematic representation of the complementarity of each of these host target binding sites to VGAM139 RNA, herein designated VGAM RNA, are described hereinbelow with reference to Table 2.

As mentioned hereinabove with reference to FIG. 1, a function of VGAM139 gene, herein designated VGAM is inhibition of expression of VGAM139 target genes. It is appreciated that specific functions, and accordingly utilities, of VGAM139 correlate with, and may be deduced from, the identity of the target genes which VGAM139 binds and inhibits, and the function of these target genes, as elaborated hereinbelow.

Sodium Channel, Voltage-gated, Type I, Alpha Polypeptide (SCN1A, Accession XM_{—}114281) is a VGAM139 host target gene. SCN1A BINDING SITE is HOST TARGET binding site found in the 3′ untranslated region of mRNA encoded by SCN1A, corresponding to a HOST TARGET binding site such as BINDING SITE I, BINDING SITE II or BINDING SITE III. Table 2 illustrates the complementarity of the nucleotide sequences of SCN1A BINDING SITE, designated SEQ ID:3451, to the nucleotide sequence of VGAM139 RNA, herein designated VGAM RNA, also designated SEQ ID:474.

A function of VGAM139 is therefore inhibition of Sodium Channel, Voltage-gated, Type I, Alpha Polypeptide (SCN1A, Accession XM_{—}114281). Accordingly, utilities of VGAM139 include diagnosis, prevention and treatment of diseases and clinical conditions associated with SCN1A. FLJ13315 (Accession NM_{—}025005) is another VGAM139 host target gene. FLJ13315 BINDING SITE is HOST TARGET binding site found in the 5′ untranslated region of mRNA encoded by FLJ13315, corresponding to a HOST TARGET binding site such as BINDING SITE I, BINDING SITE II or BINDING SITE III. Table 2 illustrates the complementarity of the nucleotide sequences of FLJ13315 BINDING SITE, designated SEQ ID:2127, to the nucleotide sequence of VGAM139 RNA, herein designated VGAM RNA, also designated SEQ ID:474.

Another function of VGAM139 is therefore inhibition of FLJ13315 (Accession NM_{—}025005). Accordingly, utilities of VGAM139 include diagnosis, prevention and treatment of diseases and clinical conditions associated with FLJ13315. KIAA0940 (Accession NM_{—}014912) is another VGAM139 host target gene. KIAA0940 BINDING SITE is HOST TARGET binding site found in the 3′ untranslated region of mRNA encoded by KIAA0940, corresponding to a HOST TARGET binding site such as BINDING SITE I, BINDING SITE II or BINDING SITE III. Table 2 illustrates the complementarity of the nucleotide sequences of KIAA0940 BINDING SITE, designated SEQ ID:1584, to the nucleotide sequence of VGAM139 RNA, herein designated VGAM RNA, also designated SEQ ID:474.

Another function of VGAM139 is therefore inhibition of KIAA0940 (Accession NM_{—}014912). Accordingly, utilities of VGAM139 include diagnosis, prevention and treatment of diseases and clinical conditions associated with KIAA0940. SARM (Accession NM_{—}015077) is another VGAM139 host target gene. SARM BINDING SITE is HOST TARGET binding site found in the 3′ untranslated region of mRNA encoded by SARM, corresponding to a HOST TARGET binding site such as BINDING SITE I, BINDING SITE II or BINDING SITE III. Table 2 illustrates the complementarity of the nucleotide sequences of SARM BINDING SITE, designated SEQ ID: 1608, to the nucleotide sequence of VGAM139 RNA, herein designated VGAM RNA, also designated SEQ ID:474.

Another function of VGAM139 is therefore inhibition of SARM (Accession NM_{—}015077). Accordingly, utilities of VGAM139 include diagnosis, prevention and treatment of diseases and clinical conditions associated with SARM. LOC144231 (Accession XM_{—}096561) is another VGAM139 host target gene. LOC144231 BINDING SITE is HOST TARGET binding site found in the 5′ untranslated region of mRNA encoded by LOC144231, corresponding to a HOST TARGET binding site such as BINDING SITE I, BINDING SITE II or BINDING SITE III. Table 2 illustrates the complementarity of the nucleotide sequences of LOC144231 BINDING SITE, designated SEQ ID:3266, to the nucleotide sequence of VGAM139 RNA, herein designated VGAM RNA, also designated SEQ ID:474.

Another function of VGAM139 is therefore inhibition of LOC144231 (Accession XM_{—}096561). Accordingly, utilities of VGAM139 include diagnosis, prevention and treatment of diseases and clinical conditions associated with LOC144231. LOC148734 (Accession XM_{—}086294) is another VGAM139 host target gene. LOC148734 BINDING SITE is HOST TARGET binding site found in the 3′ untranslated region of mRNA encoded by LOC148734, corresponding to a HOST TARGET binding site such as BINDING SITE I, BINDING SITE II or BINDING SITE III. Table 2 illustrates the complementarity of the nucleotide sequences of LOC148734 BINDING SITE, designated SEQ ID:3124, to the nucleotide sequence of VGAM139 RNA, herein designated VGAM RNA, also designated SEQ ID:474.

Another function of VGAM139 is therefore inhibition of LOC148734 (Accession XM_{—}086294). Accordingly, utilities of VGAM139 include diagnosis, prevention and treatment of diseases and clinical conditions associated with LOC148734. FIG. 1 further provides a conceptual description of a novel bioinformatically detected viral gene of the present invention, referred to here as Viral Genomic Address Messenger 140 (VGAM140) viral gene, which modulates expression of respective host target genes thereof, the function and utility of which host target genes is known in the art.

VGAM140 is a novel bioinformatically detected regulatory, non protein coding, viral micro RNA (miRNA) gene. The method by which VGAM140 was detected is described hereinabove with reference to FIGS. 1-8.

VGAM140 gene, herein designated VGAM GENE, is a viral gene contained in the genome of Vaccinia Virus. VGAM140 host target gene, herein designated VGAM HOST TARGET GENE, is a human gene contained in the human genome.

VGAM140 gene encodes a VGAM140 precursor RNA, herein designated VGAM PRECURSOR RNA. Similar to other miRNA genes, and unlike most ordinary genes, VGAM140 precursor RNA does not encode a protein. A nucleotide sequence identical or highly similar to the nucleotide sequence of VGAM140 precursor RNA is designated SEQ ID:126, and is provided hereinbelow with reference to the sequence listing part. Nucleotide sequence SEQ ID:126 is located at position 62804 relative to the genome of Vaccinia Virus.

VGAM140 precursor RNA folds onto itself, forming VGAM140 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM140 folded precursor RNA into VGAM140 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM140 RNA is designated SEQ ID:475, and is provided hereinbelow with reference to the sequence listing part.

VGAM140 host target gene, herein designated VGAM HOST TARGET GENE, encodes a corresponding messenger RNA, VGAM140 host target RNA, herein designated VGAM HOST TARGET RNA. VGAM140 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM140 RNA, herein designated VGAM RNA, binds complementarily to one or more host target binding sites located in untranslated regions of VGAM140 host target RNA, herein designated VGAM HOST TARGET RNA. This complementary binding is due to the fact that the nucleotide sequence of VGAM140 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM140 RNA, herein designated VGAM RNA, may have a different number of host target binding sites in untranslated regions of a VGAM140 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM140 RNA, herein designated VGAM RNA, to host target binding sites on VGAM140 host target RNA, herein designated VGAM HOST TARGET RNA, such as BINDING SITE I, BINDING SITE II and BINDING SITE III, inhibits translation of VGAM140 host target RNA into VGAM140 host target protein, herein designated VGAM HOST TARGET PROTEIN. VGAM host target protein is therefore outlined by a broken line.

It is appreciated that VGAM140 host target gene, herein designated VGAM HOST TARGET GENE, in fact represents a plurality of VGAM140 host target genes. The mRNA of each one of this plurality of VGAM140 host target genes comprises one or more host target binding sites, each having a nucleotide sequence which is at least partly complementary to VGAM140 RNA, herein designated VGAM RNA, and which when bound by VGAM140 RNA causes inhibition of translation of respective one or more VGAM140 host target proteins.

It is further appreciated by one skilled in the art that the mode of translational inhibition illustrated by FIG. 1 with specific reference to translational inhibition exerted by VGAM140 gene, herein designated VGAM GENE, on one or more VGAM140 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM140 is inhibition of expression of host target genes, as part of a novel viral mechanism of attacking a host. Accordingly, utilities of VGAM140 include diagnosis, prevention and treatment of viral infection by Vaccinia Virus. Specific functions, and accordingly utilities, of VGAM140 correlate with, and may be deduced from, the identity of the host target genes which VGAM140 binds and inhibits, and the function of these host target genes, as elaborated hereinbelow.

Nucleotide sequences of the VGAM140 precursor RNA, herein designated VGAM PRECURSOR RNA, and of the ‘diced’ VGAM140 RNA, herein designated VGAM RNA, and a schematic representation of the secondary folding of VGAM140 folded precursor RNA, herein designated VGAM FOLDED PRECURSOR RNA, of VGAM140 are further described hereinbelow with reference to Table 1.

Nucleotide sequences of host target binding sites, such as BINDING SITE-I, BINDING SITE-II and BINDING SITE-III of FIG. 1, found on VGAM140 host target RNA, and schematic representation of the complementarity of each of these host target binding sites to VGAM140 RNA, herein designated VGAM RNA, are described hereinbelow with reference to Table 2.

As mentioned hereinabove with reference to FIG. 1, a function of VGAM140 gene, herein designated VGAM is inhibition of expression of VGAM140 target genes. It is appreciated that specific functions, and accordingly utilities, of VGAM140 correlate with, and may be deduced from, the identity of the target genes which VGAM140 binds and inhibits, and the function of these target genes, as elaborated hereinbelow.

Methyl-CpG Binding Domain Protein 1 (MBD1, Accession NM_{—}002384) is a VGAM140 host target gene. MBD1 BINDING SITE1 through MBD1 BINDING SITE4 are HOST TARGET binding sites found in untranslated regions of mRNA encoded by MBD1, corresponding to HOST TARGET binding sites such as BINDING SITE I, BINDING SITE II or BINDING SITE III. Table 2 illustrates the complementarity of the nucleotide sequences of MBD1 BINDING SITE1 through MBD1 BINDING SITE4, designated SEQ ID:924, SEQ ID: 1644, SEQ ID: 1645 and SEQ ID: 1646 respectively, to the nucleotide sequence of VGAM140 RNA, herein designated VGAM RNA, also designated SEQ ID:475.

A function of VGAM140 is therefore inhibition of Methyl-CpG Binding Domain Protein 1 (MBD1, Accession NM_{—}002384), a gene which bind specifically to methylated DNA via a methyl-CpG-binding domain (MBD). Accordingly, utilities of VGAM140 include diagnosis, prevention and treatment of diseases and clinical conditions associated with MBD1. The function of MBD1 has been established by previous studies. Attempts to understand how DNA methylation prevents transcription have centered on 2 alternative mechanisms: direct interference of site-specific methylation with the binding of essential transcription factors, and indirect interference of promoter-proximal methylation with transcription via a protein that binds to methylated DNA. Methyl-CpG-binding protein-1 (MECP1) binds to a variety of methylated sequences in vitro, provided they contain at least 12 symmetrically methylated CpGs. MECP1 has been detected in crude nuclear extracts. Boyes and Bird (1991) and Levine et al. (1991) presented evidence suggesting that the MECP1 protein is a mediator of repression. Methylation of cytosines within the sequence CpG is essential for mouse development and has been linked to transcriptional suppression in vertebrate systems. Methyl-CpG-binding proteins MECP1 and MECP2 (OMIM Ref. No. 300005) bind preferentially to methylated DNA and can inhibit transcription. The rat Mecp2 gene was cloned by Nan et al. (1993) and its methyl-CpG-binding domain (MBD) defined. By searching DNA sequence databases with the MBD sequence, Cross et al. (1997) identified a human cDNA with potential to encode an MBD-like region. Sequencing of the complete cDNA revealed that the open reading frame also encodes 2 cysteine-rich domains that were found in animal DNA methyltransferases (see OMIM Ref. No. DNMT; 126375) and in the mammalian HRX protein, also known as MLL and ALL-1 (OMIM Ref. No. 159555). They designated the protein PCM1 for ‘protein containing MBD.’ Expressed in bacteria, it showed specific binding to methylated DNA. PCM1 also repressed transcription in vitro in a methylation-dependent manner. A polyclonal antibody raised against the protein was able to bind the native MECP1 complex from HeLa cells, indicating that PCM1 is a component of mammalian MECP1. Using PCR on a hybrid panel and FISH, Hendrich et al. (1999) mapped the MBD1 gene to chromosome 18q21, 2.1 cM distal to MBD2 (OMIM Ref. No. 603547). They mapped the murine gene to chromosome 18.

Full details of the abovementioned studies are described in the following publications, the disclosure of which are hereby incorporated by reference:
Boyes, J.; Bird, A.: DNA methylation inhibits transcription indirectly via a methyl-CpG binding protein. Cell 64: 1123-1134, 1991.; and
Levine, A.; Cantoni, G. L.; Razin, A.: Inhibition of promoter activity by methylation: possible involvement of protein mediators. Proc. Nat. Acad. Sci. 88: 6515-6518, 1991.

Further studies establishing the function and utilities of MBD1 are found in John Hopkins OMIM database record ID 156535, and in sited publications numbered 500-50 and 3056-3058 listed in the bibliography section hereinbelow, which are also hereby incorporated by reference. LOC51026 (Accession NM_{—}016072) is another VGAM140 host target gene. LOC51026 BINDING SITE is HOST TARGET binding site found in the 3′ untranslated region of mRNA encoded by LOC51026, corresponding to a HOST TARGET binding site such as BINDING SITE I, BINDING SITE II or BINDING SITE III. Table 2 illustrates the complementarity of the nucleotide sequences of LOC51026 BINDING SITE, designated SEQ ID:1659, to the nucleotide sequence of VGAM140 RNA, herein designated VGAM RNA, also designated SEQ ID:475.

Another function of VGAM140 is therefore inhibition of LOC51026 (Accession NM_{—}016072). Accordingly, utilities of VGAM140 include diagnosis, prevention and treatment of diseases and clinical conditions associated with LOC51026. FIG. 1 further provides a conceptual description of a novel bioinformatically detected viral gene of the present invention, referred to here as Viral Genomic Address Messenger 141 (VGAM141) viral gene, which modulates expression of respective host target genes thereof, the function and utility of which host target genes is known in the art.

VGAM141 is a novel bioinformatically detected regulatory, non protein coding, viral micro RNA (miRNA) gene. The method by which VGAM141 was detected is described hereinabove with reference to FIGS. 1-8.

VGAM141 gene, herein designated VGAM GENE, is a viral gene contained in the genome of Vaccinia Virus. VGAM141 host target gene, herein designated VGAM HOST TARGET GENE, is a human gene contained in the human genome.

VGAM141 gene encodes a VGAM141 precursor RNA, herein designated VGAM PRECURSOR RNA. Similar to other miRNA genes, and unlike most ordinary genes, VGAM141 precursor RNA does not encode a protein. A nucleotide sequence identical or highly similar to the nucleotide sequence of VGAM141 precursor RNA is designated SEQ ID:127, and is provided hereinbelow with reference to the sequence listing part. Nucleotide sequence SEQ ID:127 is located at position 62395 relative to the genome of Vaccinia Virus.

VGAM141 precursor RNA folds onto itself, forming VGAM141 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM141 folded precursor RNA into VGAM141 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM141 RNA is designated SEQ ID:476, and is provided hereinbelow with reference to the sequence listing part.

VGAM141 host target gene, herein designated VGAM HOST TARGET GENE, encodes a corresponding messenger RNA, VGAM141 host target RNA, herein designated VGAM HOST TARGET RNA. VGAM141 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM141 RNA, herein designated VGAM RNA, binds complementarily to one or more host target binding sites located in untranslated regions of VGAM141 host target RNA, herein designated VGAM HOST TARGET RNA. This complementary binding is due to the fact that the nucleotide sequence of VGAM141 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM141 RNA, herein designated VGAM RNA, may have a different number of host target binding sites in untranslated regions of a VGAM141 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM141 RNA, herein designated VGAM RNA, to host target binding sites on VGAM141 host target RNA, herein designated VGAM HOST TARGET RNA, such as BINDING SITE I, BINDING SITE II and BINDING SITE III, inhibits translation of VGAM141 host target RNA into VGAM141 host target protein, herein designated VGAM HOST TARGET PROTEIN. VGAM host target protein is therefore outlined by a broken line.

It is appreciated that VGAM141 host target gene, herein designated VGAM HOST TARGET GENE, in fact represents a plurality of VGAM141 host target genes. The mRNA of each one of this plurality of VGAM141 host target genes comprises one or more host target binding sites, each having a nucleotide sequence which is at least partly complementary to VGAM141 RNA, herein designated VGAM RNA, and which when bound by VGAM141 RNA causes inhibition of translation of respective one or more VGAM141 host target proteins.

It is further appreciated by one skilled in the art that the mode of translational inhibition illustrated by FIG. 1 with specific reference to translational inhibition exerted by VGAM141 gene, herein designated VGAM GENE, on one or more VGAM141 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM141 is inhibition of expression of host target genes, as part of a novel viral mechanism of attacking a host. Accordingly, utilities of VGAM141 include diagnosis, prevention and treatment of viral infection by Vaccinia Virus. Specific functions, and accordingly utilities, of VGAM141 correlate with, and may be deduced from, the identity of the host target genes which VGAM141 binds and inhibits, and the function of these host target genes, as elaborated hereinbelow.

Nucleotide sequences of the VGAM141 precursor RNA, herein designated VGAM PRECURSOR RNA, and of the ‘diced’ VGAM141 RNA, herein designated VGAM RNA, and a schematic representation of the secondary folding of VGAM141 folded precursor RNA, herein designated VGAM FOLDED PRECURSOR RNA, of VGAM141 are further described hereinbelow with reference to Table 1.

Nucleotide sequences of host target binding sites, such as BINDING SITE-I, BINDING SITE-II and BINDING SITE-III of FIG. 1, found on VGAM141 host target RNA, and schematic representation of the complementarity of each of these host target binding sites to VGAM141 RNA, herein designated VGAM RNA, are described hereinbelow with reference to Table 2.

As mentioned hereinabove with reference to FIG. 1, a function of VGAM141 gene, herein designated VGAM is inhibition of expression of VGAM141 target genes. It is appreciated that specific functions, and accordingly utilities, of VGAM141 correlate with, and may be deduced from, the identity of the target genes which VGAM141 binds and inhibits, and the function of these target genes, as elaborated hereinbelow.

Activation-induced Cytidine Deaminase (AICDA, Accession NM_{—}020661) is a VGAM141 host target gene. AICDA BINDING SITE is HOST TARGET binding site found in the 3′ untranslated region of mRNA encoded by AICDA, corresponding to a HOST TARGET binding site such as BINDING SITE I, BINDING SITE II or BINDING SITE III. Table 2 illustrates the complementarity of the nucleotide sequences of AICDA BINDING SITE, designated SEQ ID:1923, to the nucleotide sequence of VGAM141 RNA, herein designated VGAM RNA, also designated SEQ ID:476.

A function of VGAM141 is therefore inhibition of Activation-induced Cytidine Deaminase (AICDA, Accession NM_{—}020661), a gene which a member of the cytidine deaminase family. Accordingly, utilities of VGAM141 include diagnosis, prevention and treatment of diseases and clinical conditions associated with AICDA. The function of AICDA and its association with various diseases and clinical conditions, has been established by previous studies, as described hereinabove with reference to VGAM31. Glucagon (GCG, Accession NM_{—}002054) is another VGAM141 host target gene. GCG BINDING SITE is HOST TARGET binding site found in the 3′ untranslated region of mRNA encoded by GCG, corresponding to a HOST TARGET binding site such as BINDING SITE I, BINDING SITE II or BINDING SITE III. Table 2 illustrates the complementarity of the nucleotide sequences of GCG BINDING SITE, designated SEQ ID:895, to the nucleotide sequence of VGAM141 RNA, herein designated VGAM RNA, also designated SEQ ID:476.

Another function of VGAM141 is therefore inhibition of Glucagon (GCG, Accession NM_{—}002054). Accordingly, utilities of VGAM141 include diagnosis, prevention and treatment of diseases and clinical conditions associated with GCG. Protein Tyrosine Phosphatase, Non-receptor Type 1 (PTPN1, Accession NM_{—}002827) is another VGAM141 host target gene. PTPN1 BINDING SITE is HOST TARGET binding site found in the 3′ untranslated region of mRNA encoded by PTPN1, corresponding to a HOST TARGET binding site such as BINDING SITE I, BINDING SITE II or BINDING SITE III. Table 2 illustrates the complementarity of the nucleotide sequences of PTPN1 BINDING SITE, designated SEQ ID:960, to the nucleotide sequence of VGAM141 RNA, herein designated VGAM RNA, also designated SEQ ID:476.

Another function of VGAM141 is therefore inhibition of Protein Tyrosine Phosphatase, Non-receptor Type 1 (PTPN1, Accession NM_{—}002827), a gene which is a non-receptor type 1 protein tyrosine phosphatase and inhibits insulin signaling. Accordingly, utilities of VGAM141 include diagnosis, prevention and treatment of diseases and clinical conditions associated with PTPN1. The function of PTPN1 has been established by previous studies. PTP1B inhibits insulin signaling and, when overexpressed, plays a role in insulin resistance (Ahmad et al., 1997). In the 3-prime untranslated region of the PTP1B gene, Di Paola et al. (2002) identified a 1484insG variation (176885.0001) that, in 2 different populations, was associated with several features of insulin resistance. Similar data were obtained in a family-based association study by use of sib pairs discordant for genotype (Gu et al., 2000). Subjects carrying the 1484insG variant showed PTP1B mRNA overexpression in skeletal muscle. PTP1B mRNA stability was significantly higher in human embryonic kidney cells transfected with 1484insG PTP1B as compared with those transfected with wildtype PTP1B. The data indicated that the 1484insG allele causes PTP1B overexpression and plays a role in insulin resistance. Therefore, individuals carrying the 1484insG variant might particularly benefit from PTP1B inhibitors in the treatment of insulin resistance (Kennedy and Ramachandran, 2000). Animal model experiments lend further support to the function of PTPN1. Elchebly et al. (1999) generated PTP1B-deficient mice by targeted disruption of the mouse homolog of the PTP1B gene. Mice were phenotypically and pathologically normal and had normal life span. In the fed state, homozygous mutant mice had slightly lower blood glucose concentrations, and half the circulating insulin concentrations, of wildtype littermates. The enhanced insulin sensitivity of PTP1B-deficient mice was also evident in glucose- and insulin-tolerance tests. After insulin injection, deficient mice showed increased phosphorylation of the insulin receptor in liver and muscle tissue compared to wildtype mice. On a high-fat diet, PTP1B-deficient mice were resistant to weight gain and remained insulin sensitive, while wildtype mice rapidly gained weight and became insulin resistant. These results suggested a major role for PTP1B in modulation of insulin sensitivity and fuel metabolism. The authors proposed PTP1B as a potential therapeutic target for the treatment of type 2 diabetes and obesity.

It is appreciated that the abovementioned animal model for PTPN1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Elchebly, M.; Payette, P.; Michaliszyn, E.; Cromlish, W.; Collins, S.; Loy, A. L.; Normandin, D.; Cheng, A.; Himms-Hagen, J.; Chan, C.-C.; Ramachandran, C.; Gresser, M. J.; Tremblay, M. L.; Kennedy, B. P.: Increased insulin sensitivity and obesity resistance in mice lacking the protein tyrosine phosphatase-1B gene. Science 283: 1544-1548, 1999.; and
Di Paola, R.; Frittitta, L.; Miscio, G.; Bozzali, M.; Baratta, R.; Centra, M.; Spampinato, D.; Santagati, M. G.; Ercolino, T.; Cisternino, C.; Soccio, T. Mastroianno, S.; Tassi, V.; Alm.

Further studies establishing the function and utilities of PTPN1 are found in John Hopkins OMIM database record ID 176885, and in sited publications numbered 2505-2508, 2448-2405, 2499, 240 and 2409 listed in the bibliography section hereinbelow, which are also hereby incorporated by reference. SCO Cytochrome Oxidase Deficient Homolog 2 (yeast) (SCO2, Accession NM_{—}005138) is another VGAM141 host target gene. SCO2 BINDING SITE is HOST TARGET binding site found in the 5′ untranslated region of mRNA encoded by SCO2, corresponding to a HOST TARGET binding site such as BINDING SITE I, BINDING SITE II or BINDING SITE III. Table 2 illustrates the complementarity of the nucleotide sequences of SCO2 BINDING SITE, designated SEQ ID:1189, to the nucleotide sequence of VGAM141 RNA, herein designated VGAM RNA, also designated SEQ ID:476.

Another function of VGAM141 is therefore inhibition of SCO Cytochrome Oxidase Deficient Homolog 2 (yeast) (SCO2, Accession NM_{—}005138). Accordingly, utilities of VGAM141 include diagnosis, prevention and treatment of diseases and clinical conditions associated with SCO2. Uracil-DNA Glycosylase (UNG, Accession NM_{—}080911) is another VGAM141 host target gene. UNG BINDING SITE1 and UNG BINDING SITE2 are HOST TARGET binding sites found in untranslated regions of mRNA encoded by UNG, corresponding to HOST TARGET binding sites such as BINDING SITE I, BINDING SITE II or BINDING SITE III. Table 2 illustrates the complementarity of the nucleotide sequences of UNG BINDING SITE1 and UNG BINDING SITE2, designated SEQ ID:2394 and SEQ ID: 1013 respectively, to the nucleotide sequence of VGAM141 RNA, herein designated VGAM RNA, also designated SEQ ID:476.

Another function of VGAM141 is therefore inhibition of Uracil-DNA Glycosylase (UNG, Accession NM_{—}080911), a gene which excises uracil residues from the dna to prevent mutagenesis and initiate the base-excision repair (BER) pathway. Accordingly, utilities of VGAM141 include diagnosis, prevention and treatment of diseases and clinical conditions associated with UNG. The function of UNG has been established by previous studies. Uracil DNA glycosylase removes uracil in DNA resulting from deamination of cytosine or replicative incorporation of dUMP instead of dTMP (Haug et al., 1996). Animal model experiments lend further support to the function of UNG. Nilsen et al. (2000) generated knockout mice lacking Ung. In contrast to Ung − mutants of bacteria and yeast, these mice did not exhibit a greatly increased spontaneous mutation frequency. There was, however, only slow removal of uracil from misincorporated dUMP in isolated Ung −/− nuclei and an elevated steady-state level of uracil in DNA in dividing Ung −/− cells. A backup uracil-excising activity in tissue extracts from Ung null mice, with properties indistinguishable from the mammalian SMUG1 DNA glycosylase, may account for the repair of premutagenic U:G mispairs resulting from cytosine deamination in vivo. The authors suggested that the nuclear UNG protein has evolved a specialized role in mammalian cells counteracting U:A base pairs formed by use of dUTP during DNA synthesis.

It is appreciated that the abovementioned animal model for UNG is acknowledged by those skilled in the art as a scientifically valid animal model, as can be further appreciated from the publications sited hereinbelow.

Full details of the abovementioned studies are described in the following publications, the disclosure of which are hereby incorporated by reference:
Haug, T.; Skorpen, F.; Kvaloy, K.; Eftedal, I.; Lund, H.; Krokan, H. E.: Human uracil-DNA glycosylase gene: sequence organization, methylation pattern, and mapping to chromosome 12q23-q24.1. Genomics 36: 408-416, 1996.; and
Nilsen, H.; Rosewell, I.; Robins, P.; Skjelbred, C. F.; Andersen, S.; Slupphaug, G.; Daly, G.; Krokan, H. E.; Lindahl, T.; Barnes, D. E.: Uracil-DNA glycosylase (UNG)-deficient mice rev.

Further studies establishing the function and utilities of UNG are found in John Hopkins OMIM database record ID 191525, and in sited publications numbered 3036-3043 listed in the bibliography section hereinbelow, which are also hereby incorporated by reference. DKFZP761G1913 (Accession NM_{—}031474) is another VGAM141 host target gene. DKFZP761G1913 BINDING SITE is HOST TARGET binding site found in the 3′ untranslated region of mRNA encoded by DKFZP761G1913, corresponding to a HOST TARGET binding site such as BINDING SITE I, BINDING SITE II or BINDING SITE III. Table 2 illustrates the complementarity of the nucleotide sequences of DKFZP761G1913 BINDING SITE, designated SEQ ID:2209, to the nucleotide sequence of VGAM141 RNA, herein designated VGAM RNA, also designated SEQ ID:476.

Another function of VGAM141 is therefore inhibition of DKFZP761G1913 (Accession NM_{—}031474). Accordingly, utilities of VGAM141 include diagnosis, prevention and treatment of diseases and clinical conditions associated with DKFZP761G1913. KIAA0057 (Accession NM_{—}012288) is another VGAM141 host target gene. KIAA0057 BINDING SITE is HOST TARGET binding site found in the 3′ untranslated region of mRNA encoded by KIAA0057, corresponding to a HOST TARGET binding site such as BINDING SITE I, BINDING SITE II or BINDING SITE III. Table 2 illustrates the complementarity of the nucleotide sequences of KIAA0057 BINDING SITE, designated SEQ ID:1422, to the nucleotide sequence of VGAM141 RNA, herein designated VGAM RNA, also designated SEQ ID:476.

Another function of VGAM141 is therefore inhibition of KIAA0057 (Accession NM_{—}012288). Accordingly, utilities of VGAM141 include diagnosis, prevention and treatment of diseases and clinical conditions associated with KIAA0057. RTC Domain Containing 1 (RTCD1, Accession NM_{—}003729) is another VGAM141 host target gene. RTCD1 BINDING SITE is HOST TARGET binding site found in the 3′ untranslated region of mRNA encoded by RTCD1, corresponding to a HOST TARGET binding site such as BINDING SITE I, BINDING SITE II or BINDING SITE III. Table 2 illustrates the complementarity of the nucleotide sequences of RTCD1 BINDING SITE, designated SEQ ID:1053, to the nucleotide sequence of VGAM141 RNA, herein designated VGAM RNA, also designated SEQ ID:476.

Another function of VGAM141 is therefore inhibition of RTC Domain Containing 1 (RTCD1, Accession NM_{—}003729). Accordingly, utilities of VGAM141 include diagnosis, prevention and treatment of diseases and clinical conditions associated with RTCD1. LOC127396 (Accession XM_{—}059139) is another VGAM141 host target gene. LOC127396 BINDING SITE is HOST TARGET binding site found in the 3′ untranslated region of mRNA encoded by LOC127396, corresponding to a HOST TARGET binding site such as BINDING SITE I, BINDING SITE II or BINDING SITE III. Table 2 illustrates the complementarity of the nucleotide sequences of LOC127396 BINDING SITE, designated SEQ ID:3005, to the nucleotide sequence of VGAM141 RNA, herein designated VGAM RNA, also designated SEQ ID:476.

Another function of VGAM141 is therefore inhibition of LOC127396 (Accession XM_{—}059139). Accordingly, utilities of VGAM141 include diagnosis, prevention and treatment of diseases and clinical conditions associated with LOC127396. LOC130074 (Accession XM_{—}072228) is another VGAM141 host target gene. LOC130074 BINDING SITE is HOST TARGET binding site found in the 3′ untranslated region of mRNA encoded by LOC130074, corresponding to a HOST TARGET binding site such as BINDING SITE I, BINDING SITE II or BINDING SITE III. Table 2 illustrates the complementarity of the nucleotide sequences of LOC130074 BINDING SITE, designated SEQ ID:3044, to the nucleotide sequence of VGAM141 RNA, herein designated VGAM RNA, also designated SEQ ID:476.

Another function of VGAM141 is therefore inhibition of LOC130074 (Accession XM_{—}072228). Accordingly, utilities of VGAM141 include diagnosis, prevention and treatment of diseases and clinical conditions associated with LOC130074. LOC149134 (Accession XM_{—}097594) is another VGAM141 host target gene. LOC149134 BINDING SITE is HOST TARGET binding site found in the 5′ untranslated region of mRNA encoded by LOC149134, corresponding to a HOST TARGET binding site such as BINDING SITE I, BINDING SITE II or BINDING SITE III. Table 2 illustrates the complementarity of the nucleotide sequences of LOC149134 BINDING SITE, designated SEQ ID:3308, to the nucleotide sequence of VGAM141 RNA, herein designated VGAM RNA, also designated SEQ ID:476.

Another function of VGAM141 is therefore inhibition of LOC149134 (Accession XM_{—}097594). Accordingly, utilities of VGAM141 include diagnosis, prevention and treatment of diseases and clinical conditions associated with LOC149134. LOC219818 (Accession XM_{—}165589) is another VGAM141 host target gene. LOC219818 BINDING SITE is HOST TARGET binding site found in the 3′ untranslated region of mRNA encoded by LOC219818, corresponding to a HOST TARGET binding site such as BINDING SITE I, BINDING SITE II or BINDING SITE III. Table 2 illustrates the complementarity of the nucleotide sequences of LOC219818 BINDING SITE, designated SEQ ID:3503, to the nucleotide sequence of VGAM141 RNA, herein designated VGAM RNA, also designated SEQ ID:476.

Another function of VGAM141 is therefore inhibition of LOC219818 (Accession XM_{—}165589). Accordingly, utilities of VGAM141 include diagnosis, prevention and treatment of diseases and clinical conditions associated with LOC219818. LOC256714 (Accession XM_{—}172798) is another VGAM141 host target gene. LOC256714 BINDING SITE is HOST TARGET binding site found in the 3′ untranslated region of mRNA encoded by LOC256714, corresponding to a HOST TARGET binding site such as BINDING SITE I, BINDING SITE II or BINDING SITE III. Table 2 illustrates the complementarity of the nucleotide sequences of LOC256714 BINDING SITE, designated SEQ ID:3702, to the nucleotide sequence of VGAM141 RNA, herein designated VGAM RNA, also designated SEQ ID:476.

Another function of VGAM141 is therefore inhibition of LOC256714 (Accession XM_{—}172798). Accordingly, utilities of VGAM141 include diagnosis, prevention and treatment of diseases and clinical conditions associated with LOC256714. LOC91149 (Accession XM_{—}036480) is another VGAM141 host target gene. LOC91149 BINDING SITE is HOST TARGET binding site found in the 5′ untranslated region of mRNA encoded by LOC91149, corresponding to a HOST TARGET binding site such as BINDING SITE I, BINDING SITE II or BINDING SITE III. Table 2 illustrates the complementarity of the nucleotide sequences of LOC91149 BINDING SITE, designated SEQ ID:2714, to the nucleotide sequence of VGAM141 RNA, herein designated VGAM RNA, also designated SEQ ID:476.

Another function of VGAM141 is therefore inhibition of LOC91149 (Accession XM_{—}036480). Accordingly, utilities of VGAM141 include diagnosis, prevention and treatment of diseases and clinical conditions associated with LOC91149. FIG. 1 further provides a conceptual description of a novel bioinformatically detected viral gene of the present invention, referred to here as Viral Genomic Address Messenger 142 (VGAM142) viral gene, which modulates expression of respective host target genes thereof, the function and utility of which host target genes is known in the art.

VGAM142 is a novel bioinformatically detected regulatory, non protein coding, viral micro RNA (miRNA) gene. The method by which VGAM142 was detected is described hereinabove with reference to FIGS. 1-8.

VGAM142 gene, herein designated VGAM GENE, is a viral gene contained in the genome of Vaccinia Virus. VGAM142 host target gene, herein designated VGAM HOST TARGET GENE, is a human gene contained in the human genome.

VGAM142 gene encodes a VGAM142 precursor RNA, herein designated VGAM PRECURSOR RNA. Similar to other miRNA genes, and unlike most ordinary genes, VGAM142 precursor RNA does not encode a protein. A nucleotide sequence identical or highly similar to the nucleotide sequence of VGAM142 precursor RNA is designated SEQ ID:128, and is provided hereinbelow with reference to the sequence listing part. Nucleotide sequence SEQ ID:128 is located at position 67919 relative to the genome of Vaccinia Virus.

VGAM142 precursor RNA folds onto itself, forming VGAM142 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM142 folded precursor RNA into VGAM142 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM142 RNA is designated SEQ ID:477, and is provided hereinbelow with reference to the sequence listing part.

VGAM142 host target gene, herein designated VGAM HOST TARGET GENE, encodes a corresponding messenger RNA, VGAM142 host target RNA, herein designated VGAM HOST TARGET RNA. VGAM142 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM142 RNA, herein designated VGAM RNA, binds complementarily to one or more host target binding sites located in untranslated regions of VGAM142 host target RNA, herein designated VGAM HOST TARGET RNA. This complementary binding is due to the fact that the nucleotide sequence of VGAM142 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM142 RNA, herein designated VGAM RNA, may have a different number of host target binding sites in untranslated regions of a VGAM142 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM142 RNA, herein designated VGAM RNA, to host target binding sites on VGAM142 host target RNA, herein designated VGAM HOST TARGET RNA, such as BINDING SITE I, BINDING SITE II and BINDING SITE III, inhibits translation of VGAM142 host target RNA into VGAM142 host target protein, herein designated VGAM HOST TARGET PROTEIN. VGAM host target protein is therefore outlined by a broken line.

It is appreciated that VGAM142 host target gene, herein designated VGAM HOST TARGET GENE, in fact represents a plurality of VGAM142 host target genes. The mRNA of each one of this plurality of VGAM142 host target genes comprises one or more host target binding sites, each having a nucleotide sequence which is at least partly complementary to VGAM142 RNA, herein designated VGAM RNA, and which when bound by VGAM142 RNA causes inhibition of translation of respective one or more VGAM142 host target proteins.

It is further appreciated by one skilled in the art that the mode of translational inhibition illustrated by FIG. 1 with specific reference to translational inhibition exerted by VGAM142 gene, herein designated VGAM GENE, on one or more VGAM142 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM142 is inhibition of expression of host target genes, as part of a novel viral mechanism of attacking a host. Accordingly, utilities of VGAM142 include diagnosis, prevention and treatment of viral infection by Vaccinia Virus. Specific functions, and accordingly utilities, of VGAM142 correlate with, and may be deduced from, the identity of the host target genes which VGAM142 binds and inhibits, and the function of these host target genes, as elaborated hereinbelow.

Nucleotide sequences of the VGAM142 precursor RNA, herein designated VGAM PRECURSOR RNA, and of the ‘diced’ VGAM142 RNA, herein designated VGAM RNA, and a schematic representation of the secondary folding of VGAM142 folded precursor RNA, herein designated VGAM FOLDED PRECURSOR RNA, of VGAM142 are further described hereinbelow with reference to Table 1.

Nucleotide sequences of host target binding sites, such as BINDING SITE-I, BINDING SITE-II and BINDING SITE-III of FIG. 1, found on VGAM142 host target RNA, and schematic representation of the complementarity of each of these host target binding sites to VGAM142 RNA, herein designated VGAM RNA, are described hereinbelow with reference to Table 2.

As mentioned hereinabove with reference to FIG. 1, a function of VGAM142 gene, herein designated VGAM is inhibition of expression of VGAM142 target genes. It is appreciated that specific functions, and accordingly utilities, of VGAM142 correlate with, and may be deduced from, the identity of the target genes which VGAM142 binds and inhibits, and the function of these target genes, as elaborated hereinbelow.

Acyl-Coenzyme A Dehydrogenase, Short/branched Chain (ACADSB, Accession NM_{—}001609) is a VGAM142 host target gene. ACADSB BINDING SITE is HOST TARGET binding site found in the 3′ untranslated region of mRNA encoded by ACADSB, corresponding to a HOST TARGET binding site such as BINDING SITE I, BINDING SITE II or BINDING SITE III. Table 2 illustrates the complementarity of the nucleotide sequences of ACADSB BINDING SITE, designated SEQ ID:838, to the nucleotide sequence of VGAM142 RNA, herein designated VGAM RNA, also designated SEQ ID:477.

A function of VGAM142 is therefore inhibition of Acyl-Coenzyme A Dehydrogenase, Short/branched Chain (ACADSB, Accession NM_{—}001609). Accordingly, utilities of VGAM142 include diagnosis, prevention and treatment of diseases and clinical conditions associated with ACADSB. Ectodysplasin 1, Anhidrotic Receptor (EDAR, Accession NM_{—}022336) is another VGAM142 host target gene. EDAR BINDING SITE is HOST TARGET binding site found in the 3′ untranslated region of mRNA encoded by EDAR, corresponding to a HOST TARGET binding site such as BINDING SITE I, BINDING SITE II or BINDING SITE III. Table 2 illustrates the complementarity of the nucleotide sequences of EDAR BINDING SITE, designated SEQ ID:1985, to the nucleotide sequence of VGAM142 RNA, herein designated VGAM RNA, also designated SEQ ID:477.

Another function of VGAM142 is therefore inhibition of Ectodysplasin 1, Anhidrotic Receptor (EDAR, Accession NM_{—}022336). Accordingly, utilities of VGAM142 include diagnosis, prevention and treatment of diseases and clinical conditions associated with EDAR. MAX Protein (MAX, Accession NM_{—}145112) is another VGAM142 host target gene. MAX BINDING SITE1 and MAX BINDING SITE2 are HOST TARGET binding sites found in untranslated regions of mRNA encoded by MAX, corresponding to HOST TARGET binding sites such as BINDING SITE I, BINDING SITE II or BINDING SITE III. Table 2 illustrates the complementarity of the nucleotide sequences of MAX BINDING SITE1 and MAX BINDING SITE2, designated SEQ ID:2515 and SEQ ID:923 respectively, to the nucleotide sequence of VGAM142 RNA, herein designated VGAM RNA, also designated SEQ ID:477.

Another function of VGAM142 is therefore inhibition of MAX Protein (MAX, Accession NM_{—}145112), a gene which interacts specifically with the MYC (190080) protein. Accordingly, utilities of VGAM142 include diagnosis, prevention and treatment of diseases and clinical conditions associated with MAX. The function of MAX has been established by previous studies. The MAX gene encodes a protein that interacts specifically with the MYC (OMIM Ref. No. 190080) protein to form a heterodimer with high affinity for the specific cognate DNA binding site of MYC. Wagner et al. (1992) demonstrated that 2 species of RNA hybridized specifically to a MAX cDNA probe in all human and murine cell lines tested. Unlike MYC, the steady state level of MAX RNA was not significantly modulated with respect to proliferation or differentiation. Unlike MYC RNA, MAX RNA was relatively stable with a half-life of more than 3 hours, and therefore it did not exhibit the characteristic short half-life of RNAs encoded by most immediate early genes. The predicted tertiary structure of MAX closely resembles that of MYC, and it was on the basis of the basic/helix-loop-helix/leucine-zipper homology that Prendergast et al. (1991) cloned the cDNA encoding MAX. Zervos et al. (1995) described MIX2 (OMIM Ref. No. 600601), a protein that interacts with the MAX protein. Grandori et al. (1996) identified DDX18 (OMIM Ref. No. 606355) as a direct in vivo target of Myc and Max and hypothesized that Myc may exert its effects on cell behavior through proteins that affect RNA structure and metabolism. By fluorescence in situ chromosomal hybridization, Wagner et al. (1992) demonstrated that the MAX gene is located in band 14q23. This region of chromosome 14 is involved in deletions in B-cell chronic lymphocytic leukemia and malignant lymphomas and in the 12;14 translocation in uterine leiomyomas. Gilladoga et al. (1992) similarly mapped the MAX gene to 14q22-q24 by isotopic in situ hybridization and to mouse chromosome 12 in region D.

Full details of the abovementioned studies are described in the following publications, the disclosure of which are hereby incorporated by reference:
Wagner, A. J.; Le Beau, M. M.; Diaz, M. O.; Hay, N.: Expression, regulation, and chromosomal localization of the Max gene. Proc. Nat. Acad. Sci. 89: 3111-3115, 1992.; and
Prendergast, G. C.; Lawe, D.; Ziff, E. B.: Association of Myn, the murine homolog of Max, with c-Myc stimulates methylation-sensitive DNA binding and Ras cotransformation. Cell 65: 395-.

Further studies establishing the function and utilities of MAX are found in John Hopkins OMIM database record ID 154950, and in sited publications numbered 686-68 and 2538-2541 listed in the bibliography section hereinbelow, which are also hereby incorporated by reference. NIMA (never in mitosis gene a)-related Kinase 4 (NEK4, Accession NM_{—}003157) is another VGAM142 host target gene. NEK4 BINDING SITE is HOST TARGET binding site found in the 3′ untranslated region of mRNA encoded by NEK4, corresponding to a HOST TARGET binding site such as BINDING SITE I, BINDING SITE II or BINDING SITE III. Table 2 illustrates the complementarity of the nucleotide sequences of NEK4 BINDING SITE, designated SEQ ID:995, to the nucleotide sequence of VGAM142 RNA, herein designated VGAM RNA, also designated SEQ ID:477.

Another function of VGAM142 is therefore inhibition of NIMA (never in mitosis gene a)-related Kinase 4 (NEK4, Accession NM_{—}003157). Accordingly, utilities of VGAM142 include diagnosis, prevention and treatment of diseases and clinical conditions associated with NEK4. Chromosome 20 Open Reading Frame 170 (C20orf170, Accession XM_{—}029885) is another VGAM142 host target gene. C20orf170 BINDING SITE is HOST TARGET binding site found in the 3′ untranslated region of mRNA encoded by C20orf170, corresponding to a HOST TARGET binding site such as BINDING SITE I, BINDING SITE II or BINDING SITE III. Table 2 illustrates the complementarity of the nucleotide sequences of C20orf170 BINDING SITE, designated SEQ ID:2608, to the nucleotide sequence of VGAM142 RNA, herein designated VGAM RNA, also designated SEQ ID:477.

Another function of VGAM142 is therefore inhibition of Chromosome 20 Open Reading Frame 170 (C20orf170, Accession XM_{—}029885). Accordingly, utilities of VGAM142 include diagnosis, prevention and treatment of diseases and clinical conditions associated with C20orf170. FLJ20435 (Accession NM_{—}017821) is another VGAM142 host target gene. FLJ20435 BINDING SITE is HOST TARGET binding site found in the 3′ untranslated region of mRNA encoded by FLJ20435, corresponding to a HOST TARGET binding site such as BINDING SITE I, BINDING SITE II or BINDING SITE III. Table 2 illustrates the complementarity of the nucleotide sequences of FLJ20435 BINDING SITE, designated SEQ ID:1756, to the nucleotide sequence of VGAM142 RNA, herein designated VGAM RNA, also designated SEQ ID:477.

Another function of VGAM142 is therefore inhibition of FLJ20435 (Accession NM_{—}017821). Accordingly, utilities of VGAM142 include diagnosis, prevention and treatment of diseases and clinical conditions associated with FLJ20435. FLJ23071 (Accession NM_{—}025192) is another VGAM142 host target gene. FLJ23071 BINDING SITE is HOST TARGET binding site found in the 3′ untranslated region of mRNA encoded by FLJ23071, corresponding to a HOST TARGET binding site such as BINDING SITE I, BINDING SITE II or BINDING SITE III. Table 2 illustrates the complementarity of the nucleotide sequences of FLJ23071 BINDING SITE, designated SEQ ID:2143, to the nucleotide sequence of VGAM142 RNA, herein designated VGAM RNA, also designated SEQ ID:477.

Another function of VGAM142 is therefore inhibition of FLJ23071 (Accession NM_{—}025192). Accordingly, utilities of VGAM142 include diagnosis, prevention and treatment of diseases and clinical conditions associated with FLJ23071. KIAA0562 (Accession NM_{—}014704) is another VGAM142 host target gene. KIAA0562 BINDING SITE is HOST TARGET binding site found in the 3′ untranslated region of mRNA encoded by KIAA0562, corresponding to a HOST TARGET binding site such as BINDING SITE I, BINDING SITE II or BINDING SITE III. Table 2 illustrates the complementarity of the nucleotide sequences of KIAA0562 BINDING SITE, designated SEQ ID:1525, to the nucleotide sequence of VGAM142 RNA, herein designated VGAM RNA, also designated SEQ ID:477.

Another function of VGAM142 is therefore inhibition of KIAA0562 (Accession NM_{—}014704). Accordingly, utilities of VGAM142 include diagnosis, prevention and treatment of diseases and clinical conditions associated with KIAA0562. Leucine Rich Repeat (in FLII) Interacting Protein 1 (LRRFIP1, Accession NM_{—}004735) is another VGAM142 host target gene. LRRFIP1 BINDING SITE is HOST TARGET binding site found in the 3′ untranslated region of mRNA encoded by LRRFIP1, corresponding to a HOST TARGET binding site such as BINDING SITE I, BINDING SITE II or BINDING SITE III. Table 2 illustrates the complementarity of the nucleotide sequences of LRRFIP1 BINDING SITE, designated SEQ ID:1151, to the nucleotide sequence of VGAM142 RNA, herein designated VGAM RNA, also designated SEQ ID:477.

Another function of VGAM142 is therefore inhibition of Leucine Rich Repeat (in FLII) Interacting Protein 1 (LRRFIP1, Accession NM_{—}004735). Accordingly, utilities of VGAM142 include diagnosis, prevention and treatment of diseases and clinical conditions associated with LRRFIP1. P115 (Accession NM_{—}003715) is another VGAM142 host target gene. P115 BINDING SITE is HOST TARGET binding site found in the 3′ untranslated region of mRNA encoded by P115, corresponding to a HOST TARGET binding site such as BINDING SITE I, BINDING SITE II or BINDING SITE III. Table 2 illustrates the complementarity of the nucleotide sequences of P115 BINDING SITE, designated SEQ ID:1051, to the nucleotide sequence of VGAM142 RNA, herein designated VGAM RNA, also designated SEQ ID:477.

Another function of VGAM142 is therefore inhibition of P115 (Accession NM_{—}003715). Accordingly, utilities of VGAM142 include diagnosis, prevention and treatment of diseases and clinical conditions associated with P115. Serologically Defined Colon Cancer Antigen 16 (SDCCAG16, Accession NM_{—}006649) is another VGAM142 host target gene. SDCCAG16 BINDING SITE is HOST TARGET binding site found in the 3′ untranslated region of mRNA encoded by SDCCAG16, corresponding to a HOST TARGET binding site such as BINDING SITE I, BINDING SITE II or BINDING SITE III. Table 2 illustrates the complementarity of the nucleotide sequences of SDCCAG16 BINDING SITE, designated SEQ ID:1316, to the nucleotide sequence of VGAM142 RNA, herein designated VGAM RNA, also designated SEQ ID:477.

Another function of VGAM142 is therefore inhibition of Serologically Defined Colon Cancer Antigen 16 (SDCCAG16, Accession NM_{—}006649). Accordingly, utilities of VGAM142 include diagnosis, prevention and treatment of diseases and clinical conditions associated with SDCCAG16. TGFB1-induced Anti-apoptotic Factor 1 (TIAF1, Accession NM_{—}078471) is another VGAM142 host target gene. TIAF1 BINDING SITE is HOST TARGET binding site found in the 3′ untranslated region of mRNA encoded by TIAF1, corresponding to a HOST TARGET binding site such as BINDING SITE I, BINDING SITE II or BINDING SITE III. Table 2 illustrates the complementarity of the nucleotide sequences of TIAF1 BINDING SITE, designated SEQ ID:2370, to the nucleotide sequence of VGAM142 RNA, herein designated VGAM RNA, also designated SEQ ID:477.

Another function of VGAM142 is therefore inhibition of TGFB1-induced Anti-apoptotic Factor 1 (TIAF1, Accession NM_{—}078471). Accordingly, utilities of VGAM142 include diagnosis, prevention and treatment of diseases and clinical conditions associated with TIAF1. LOC149711 (Accession XM_{—}097720) is another VGAM142 host target gene. LOC149711 BINDING SITE is HOST TARGET binding site found in the 3′ untranslated region of mRNA encoded by LOC149711, corresponding to a HOST TARGET binding site such as BINDING SITE I, BINDING SITE II or BINDING SITE III. Table 2 illustrates the complementarity of the nucleotide sequences of LOC149711 BINDING SITE, designated SEQ ID:3314, to the nucleotide sequence of VGAM142 RNA, herein designated VGAM RNA, also designated SEQ ID:477.

Another function of VGAM142 is therefore inhibition of LOC149711 (Accession XM_{—}097720). Accordingly, utilities of VGAM142 include diagnosis, prevention and treatment of diseases and clinical conditions associated with LOC149711. LOC169933 (Accession XM_{—}092989) is another VGAM142 host target gene. LOC169933 BINDING SITE is HOST TARGET binding site found in the 5′ untranslated region of mRNA encoded by LOC169933, corresponding to a HOST TARGET binding site such as BINDING SITE I, BINDING SITE II or BINDING SITE III. Table 2 illustrates the complementarity of the nucleotide sequences of LOC169933 BINDING SITE, designated SEQ ID:3251, to the nucleotide sequence of VGAM142 RNA, herein designated VGAM RNA, also designated SEQ ID:477.

Another function of VGAM142 is therefore inhibition of LOC169933 (Accession XM_{—}092989). Accordingly, utilities of VGAM142 include diagnosis, prevention and treatment of diseases and clinical conditions associated with LOC169933. FIG. 1 further provides a conceptual description of a novel bioinformatically detected viral gene of the present invention, referred to here as Viral Genomic Address Messenger 143 (VGAM143) viral gene, which modulates expression of respective host target genes thereof, the function and utility of which host target genes is known in the art.

VGAM143 is a novel bioinformatically detected regulatory, non protein coding, viral micro RNA (miRNA) gene. The method by which VGAM143 was detected is described hereinabove with reference to FIGS. 1-8.

VGAM143 gene, herein designated VGAM GENE, is a viral gene contained in the genome of Vaccinia Virus. VGAM143 host target gene, herein designated VGAM HOST TARGET GENE, is a human gene contained in the human genome.

VGAM143 gene encodes a VGAM143 precursor RNA, herein designated VGAM PRECURSOR RNA. Similar to other miRNA genes, and unlike most ordinary genes, VGAM143 precursor RNA does not encode a protein. A nucleotide sequence identical or highly similar to the nucleotide sequence of VGAM143 precursor RNA is designated SEQ ID:129, and is provided hereinbelow with reference to the sequence listing part. Nucleotide sequence SEQ ID:129 is located at position 69887 relative to the genome of Vaccinia Virus.

VGAM143 precursor RNA folds onto itself, forming VGAM143 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM143 folded precursor RNA into VGAM143 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 25%) nucleotide sequence of VGAM143 RNA is designated SEQ ID:478, and is provided hereinbelow with reference to the sequence listing part.

VGAM143 host target gene, herein designated VGAM HOST TARGET GENE, encodes a corresponding messenger RNA, VGAM143 host target RNA, herein designated VGAM HOST TARGET RNA. VGAM143 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM143 RNA, herein designated VGAM RNA, binds complementarily to one or more host target binding sites located in untranslated regions of VGAM143 host target RNA, herein designated VGAM HOST TARGET RNA. This complementary binding is due to the fact that the nucleotide sequence of VGAM143 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM143 RNA, herein designated VGAM RNA, may have a different number of host target binding sites in untranslated regions of a VGAM143 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM143 RNA, herein designated VGAM RNA, to host target binding sites on VGAM143 host target RNA, herein designated VGAM HOST TARGET RNA, such as BINDING SITE I, BINDING SITE II and BINDING SITE III, inhibits translation of VGAM143 host target RNA into VGAM143 host target protein, herein designated VGAM HOST TARGET PROTEIN. VGAM host target protein is therefore outlined by a broken line.

It is appreciated that VGAM143 host target gene, herein designated VGAM HOST TARGET GENE, in fact represents a plurality of VGAM143 host target genes. The mRNA of each one of this plurality of VGAM143 host target genes comprises one or more host target binding sites, each having a nucleotide sequence which is at least partly complementary to VGAM143 RNA, herein designated VGAM RNA, and which when bound by VGAM143 RNA causes inhibition of translation of respective one or more VGAM143 host target proteins.

It is further appreciated by one skilled in the art that the mode of translational inhibition illustrated by FIG. 1 with specific reference to translational inhibition exerted by VGAM143 gene, herein designated VGAM GENE, on one or more VGAM143 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM143 is inhibition of expression of host target genes, as part of a novel viral mechanism of attacking a host. Accordingly, utilities of VGAM143 include diagnosis, prevention and treatment of viral infection by Vaccinia Virus. Specific functions, and accordingly utilities, of VGAM143 correlate with, and may be deduced from, the identity of the host target genes which VGAM143 binds and inhibits, and the function of these host target genes, as elaborated hereinbelow.

Nucleotide sequences of the VGAM143 precursor RNA, herein designated VGAM PRECURSOR RNA, and of the ‘diced’ VGAM143 RNA, herein designated VGAM RNA, and a schematic representation of the secondary folding of VGAM143 folded precursor RNA, herein designated VGAM FOLDED PRECURSOR RNA, of VGAM143 are further described hereinbelow with reference to Table 1.

Nucleotide sequences of host target binding sites, such as BINDING SITE-I, BINDING SITE-II and BINDING SITE-III of FIG. 1, found on VGAM143 host target RNA, and schematic representation of the complementarity of each of these host target binding sites to VGAM143 RNA, herein designated VGAM RNA, are described hereinbelow with reference to Table 2.

As mentioned hereinabove with reference to FIG. 1, a function of VGAM143 gene, herein designated VGAM is inhibition of expression of VGAM143 target genes. It is appreciated that specific functions, and accordingly utilities, of VGAM143 correlate with, and may be deduced from, the identity of the target genes which VGAM143 binds and inhibits, and the function of these target genes, as elaborated hereinbelow.

Sorting Nexin 9 (SNX9, Accession NM_{—}016224) is a VGAM143 host target gene. SNX9 BINDING SITE is HOST TARGET binding site found in the 3′ untranslated region of mRNA encoded by SNX9, corresponding to a HOST TARGET binding site such as BINDING SITE I, BINDING SITE II or BINDING SITE III. Table 2 illustrates the complementarity of the nucleotide sequences of SNX9 BINDING SITE, designated SEQ ID: 1673, to the nucleotide sequence of VGAM143 RNA, herein designated VGAM RNA, also designated SEQ ID:478.

A function of VGAM143 is therefore inhibition of Sorting Nexin 9 (SNX9, Accession NM_{—}016224). Accordingly, utilities of VGAM143 include diagnosis, prevention and treatment of diseases and clinical conditions associated with SNX9. TOX (Accession NM_{—}014729) is another VGAM143 host target gene. TOX BINDING SITE is HOST TARGET binding site found in the 5′ untranslated region of mRNA encoded by TOX, corresponding to a HOST TARGET binding site such as BINDING SITE I, BINDING SITE II or BINDING SITE III. Table 2 illustrates the complementarity of the nucleotide sequences of TOX BINDING SITE, designated SEQ ID: 1533, to the nucleotide sequence of VGAM143 RNA, herein designated VGAM RNA, also designated SEQ ID:478.

Another function of VGAM143 is therefore inhibition of TOX (Accession NM_{—}014729). Accordingly, utilities of VGAM143 include diagnosis, prevention and treatment of diseases and clinical conditions associated with TOX. DKFZP434C212 (Accession XM_{—}044196) is another VGAM143 host target gene. DKFZP434C212 BINDING SITE is HOST TARGET binding site found in the 3′ untranslated region of mRNA encoded by DKFZP434C212, corresponding to a HOST TARGET binding site such as BINDING SITE I, BINDING SITE II or BINDING SITE III. Table 2 illustrates the complementarity of the nucleotide sequences of DKFZP434C212 BINDING SITE, designated SEQ ID:2833, to the nucleotide sequence of VGAM143 RNA, herein designated VGAM RNA, also designated SEQ ID:478.

Another function of VGAM143 is therefore inhibition of DKFZP434C212 (Accession XM_{—}044196). Accordingly, utilities of VGAM143 include diagnosis, prevention and treatment of diseases and clinical conditions associated with DKFZP434C212. LOC151438 (Accession XM_{—}098060) is another VGAM143 host target gene. LOC151438 BINDING SITE is HOST TARGET binding site found in the 5′ untranslated region of mRNA encoded by LOC151438, corresponding to a HOST TARGET binding site such as BINDING SITE I, BINDING SITE II or BINDING SITE III. Table 2 illustrates the complementarity of the nucleotide sequences of LOC151438 BINDING SITE, designated SEQ ID:3345, to the nucleotide sequence of VGAM143 RNA, herein designated VGAM RNA, also designated SEQ ID:478.

Another function of VGAM143 is therefore inhibition of LOC151438 (Accession XM_{—}098060). Accordingly, utilities of VGAM143 include diagnosis, prevention and treatment of diseases and clinical conditions associated with LOC151438. LOC221663 (Accession XM_{—}168131) is another VGAM143 host target gene. LOC221663 BINDING SITE is HOST TARGET binding site found in the 3′ untranslated region of mRNA encoded by LOC221663, corresponding to a HOST TARGET binding site such as BINDING SITE I, BINDING SITE II or BINDING SITE III. Table 2 illustrates the complementarity of the nucleotide sequences of LOC221663 BINDING SITE, designated SEQ ID:3619, to the nucleotide sequence of VGAM143 RNA, herein designated VGAM RNA, also designated SEQ ID:478.

Another function of VGAM143 is therefore inhibition of LOC221663 (Accession XM_{—}168131). Accordingly, utilities of VGAM143 include diagnosis, prevention and treatment of diseases and clinical conditions associated with LOC221663. LOC257358 (Accession XM_{—}173138) is another VGAM143 host target gene. LOC257358 BINDING SITE is HOST TARGET binding site found in the 5′ untranslated region of mRNA encoded by LOC257358, corresponding to a HOST TARGET binding site such as BINDING SITE I, BINDING SITE II or BINDING SITE III. Table 2 illustrates the complementarity of the nucleotide sequences of LOC257358 BINDING SITE, designated SEQ ID:3730, to the nucleotide sequence of VGAM143 RNA, herein designated VGAM RNA, also designated SEQ ID:478.

Another function of VGAM143 is therefore inhibition of LOC257358 (Accession XM_{—}173138). Accordingly, utilities of VGAM143 include diagnosis, prevention and treatment of diseases and clinical conditions associated with LOC257358. LOC56832 (Accession XM_{—}035950) is another VGAM143 host target gene. LOC56832 BINDING SITE is HOST TARGET binding site found in the 3′ untranslated region of mRNA encoded by LOC56832, corresponding to a HOST TARGET binding site such as BINDING SITE I, BINDING SITE II or BINDING SITE III. Table 2 illustrates the complementarity of the nucleotide sequences of LOC56832 BINDING SITE, designated SEQ ID:2703, to the nucleotide sequence of VGAM143 RNA, herein designated VGAM RNA, also designated SEQ ID:478.

Another function of VGAM143 is therefore inhibition of LOC56832 (Accession XM_{—}035950). Accordingly, utilities of VGAM143 include diagnosis, prevention and treatment of diseases and clinical conditions associated with LOC56832. FIG. 1 further provides a conceptual description of a novel bioinformatically detected viral gene of the present invention, referred to here as Viral Genomic Address Messenger 144 (VGAM144) viral gene, which modulates expression of respective host target genes thereof, the function and utility of which host target genes is known in the art.

VGAM144 is a novel bioinformatically detected regulatory, non protein coding, viral micro RNA (miRNA) gene. The method by which VGAM144 was detected is described hereinabove with reference to FIGS. 1-8.

VGAM144 gene, herein designated VGAM GENE, is a viral gene contained in the genome of Vaccinia Virus. VGAM144 host target gene, herein designated VGAM HOST TARGET GENE, is a human gene contained in the human genome.

VGAM144 gene encodes a VGAM144 precursor RNA, herein designated VGAM PRECURSOR RNA. Similar to other miRNA genes, and unlike most ordinary genes, VGAM144 precursor RNA does not encode a protein. A nucleotide sequence identical or highly similar to the nucleotide sequence of VGAM144 precursor RNA is designated SEQ ID:130, and is provided hereinbelow with reference to the sequence listing part. Nucleotide sequence SEQ ID:130 is located at position 68429 relative to the genome of Vaccinia Virus.

VGAM144 precursor RNA folds onto itself, forming VGAM144 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM144 folded precursor RNA into VGAM144 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM144 RNA is designated SEQ ID:479, and is provided hereinbelow with reference to the sequence listing part.

VGAM144 host target gene, herein designated VGAM HOST TARGET GENE, encodes a corresponding messenger RNA, VGAM144 host target RNA, herein designated VGAM HOST TARGET RNA. VGAM144 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM144 RNA, herein designated VGAM RNA, binds complementarily to one or more host target binding sites located in untranslated regions of VGAM144 host target RNA, herein designated VGAM HOST TARGET RNA. This complementary binding is due to the fact that the nucleotide sequence of VGAM144 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM144 RNA, herein designated VGAM RNA, may have a different number of host target binding sites in untranslated regions of a VGAM144 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM144 RNA, herein designated VGAM RNA, to host target binding sites on VGAM144 host target RNA, herein designated VGAM HOST TARGET RNA, such as BINDING SITE I, BINDING SITE II and BINDING SITE III, inhibits translation of VGAM144 host target RNA into VGAM144 host target protein, herein designated VGAM HOST TARGET PROTEIN. VGAM host target protein is therefore outlined by a broken line.

It is appreciated that VGAM144 host target gene, herein designated VGAM HOST TARGET GENE, in fact represents a plurality of VGAM144 host target genes. The mRNA of each one of this plurality of VGAM144 host target genes comprises one or more host target binding sites, each having a nucleotide sequence which is at least partly complementary to VGAM144 RNA, herein designated VGAM RNA, and which when bound by VGAM144 RNA causes inhibition of translation of respective one or more VGAM144 host target proteins.

It is further appreciated by one skilled in the art that the mode of translational inhibition illustrated by FIG. 1 with specific reference to translational inhibition exerted by VGAM144 gene, herein designated VGAM GENE, on one or more VGAM144 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM144 is inhibition of expression of host target genes, as part of a novel viral mechanism of attacking a host. Accordingly, utilities of VGAM144 include diagnosis, prevention and treatment of viral infection by Vaccinia Virus. Specific functions, and accordingly utilities, of VGAM144 correlate with, and may be deduced from, the identity of the host target genes which VGAM144 binds and inhibits, and the function of these host target genes, as elaborated hereinbelow.

Nucleotide sequences of the VGAM144 precursor RNA, herein designated VGAM PRECURSOR RNA, and of the ‘diced’ VGAM144 RNA, herein designated VGAM RNA, and a schematic representation of the secondary folding of VGAM144 folded precursor RNA, herein designated VGAM FOLDED PRECURSOR RNA, of VGAM144 are further described hereinbelow with reference to Table 1.

Nucleotide sequences of host target binding sites, such as BINDING SITE-I, BINDING SITE-II and BINDING SITE-III of FIG. 1, found on VGAM144 host target RNA, and schematic representation of the complementarity of each of these host target binding sites to VGAM144 RNA, herein designated VGAM RNA, are described hereinbelow with reference to Table 2.

As mentioned hereinabove with reference to FIG. 1, a function of VGAM144 gene, herein designated VGAM is inhibition of expression of VGAM144 target genes. It is appreciated that specific functions, and accordingly utilities, of VGAM144 correlate with, and may be deduced from, the identity of the target genes which VGAM144 binds and inhibits, and the function of these target genes, as elaborated hereinbelow.

SRY (sex determining region Y)-box 9 (campomelic dysplasia, autosomal sex-reversal) (SOX9, Accession NM_{—}000346) is a VGAM144 host target gene. SOX9 BINDING SITE is HOST TARGET binding site found in the 3′ untranslated region of mRNA encoded by SOX9, corresponding to a HOST TARGET binding site such as BINDING SITE I, BINDING SITE II or BINDING SITE III. Table 2 illustrates the complementarity of the nucleotide sequences of SOX9 BINDING SITE, designated SEQ ID:732, to the nucleotide sequence of VGAM144 RNA, herein designated VGAM RNA, also designated SEQ ID:479.

A function of VGAM144 is therefore inhibition of SRY (sex determining region Y)-box 9 (campomelic dysplasia, autosomal sex-reversal) (SOX9, Accession NM_{—}000346), a gene which regulates the expression of other genes involved in chondrogenesis. Accordingly, utilities of VGAM144 include diagnosis, prevention and treatment of diseases and clinical conditions associated with SOX9. The function of SOX9 has been established by previous studies. Murakami et al. (2000) showed that expression of Sox9 is up-regulated by fibroblast growth factors (FGFs; OMIM Ref. No. 601513) in primary chondrocytes and in Sox9-expressing mesenchymal cells. They further presented evidence that FGF stimulation of Sox9 expression is mediated by the mitogen-activated protein kinase (MAPK) cascade (see OMIM Ref. No. 176948) a signal transduction pathway that is activated by growth factors such as FGF. The data strongly suggested that FGF and the MAPK pathway play an important role in the regulation of Sox9 expression during chondrocyte differentiation, Bi et al. (2001) proposed that Sox9 also has a role in regulating the transition to hypertrophic chondrocytes in the growth plate. Despite the severe hypoplasia of cartilages, the overall organization and cellular composition of the growth plate were otherwise normal. The results suggested that 2 critical steps of the chondrocyte differentiation pathway are sensitive to Sox9 dosage: an early step presumably at the stage of mesenchymal condensation of cartilage primordia, and a later step preceding the transition of chondrocytes into hypertrophic chondrocytes.

Full details of the abovementioned studies are described in the following publications, the disclosure of which are hereby incorporated by reference:
Murakami, S.; Kan, M.; McKeehan, W. L.; de Crombrugghe, B.: Up-regulation of the chondrogenic Sox9 gene by fibroblast growth factors is mediated by the mitogen-activated protein kinase pathway. Proc. Nat. Acad. Sci. 97: 1113-1118, 2000.; and
Bi, W.; Huang, W.; Whitworth, D. J.; Deng, J. M.; Zhang, Z.; Behringer, R. R.; de Crombrugghe, B.: Haploinsufficiency of Sox9 results in defective cartilage primordia and premature skelet.

Further studies establishing the function and utilities of SOX9 are found in John Hopkins OMIM database record ID 114290, and in sited publications numbered 2837-2851, 2851, 2852-2859, 2526, 2871-2877, 2719, 2878-2893, 2956-2899, 2836, 2900-290 and 2812-2813 listed in the bibliography section hereinbelow, which are also hereby incorporated by reference. FLJ14356 (Accession NM_{—}030824) is another VGAM144 host target gene. FLJ14356 BINDING SITE is HOST TARGET binding site found in the 3′ untranslated region of mRNA encoded by FLJ14356, corresponding to a HOST TARGET binding site such as BINDING SITE I, BINDING SITE II or BINDING SITE III. Table 2 illustrates the complementarity of the nucleotide sequences of FLJ14356 BINDING SITE, designated SEQ ID:2171, to the nucleotide sequence of VGAM144 RNA, herein designated VGAM RNA, also designated SEQ ID:479.

Another function of VGAM144 is therefore inhibition of FLJ14356 (Accession NM_{—}030824). Accordingly, utilities of VGAM144 include diagnosis, prevention and treatment of diseases and clinical conditions associated with FLJ14356. KIAA0982 (Accession NM_{—}014023) is another VGAM144 host target gene. KIAA0982 BINDING SITE is HOST TARGET binding site found in the 3′ untranslated region of mRNA encoded by KIAA0982, corresponding to a HOST TARGET binding site such as BINDING SITE I, BINDING SITE II or BINDING SITE III. Table 2 illustrates the complementarity of the nucleotide sequences of KIAA0982 BINDING SITE, designated SEQ ID: 1464, to the nucleotide sequence of VGAM144 RNA, herein designated VGAM RNA, also designated SEQ ID:479.

Another function of VGAM144 is therefore inhibition of KIAA0982 (Accession NM_{—}014023). Accordingly, utilities of VGAM144 include diagnosis, prevention and treatment of diseases and clinical conditions associated with KIAA0982. KIAA1028 (Accession XM_{—}166324) is another VGAM144 host target gene. KIAA1028 BINDING SITE is HOST TARGET binding site found in the 3′ untranslated region of mRNA encoded by KIAA1028, corresponding to a HOST TARGET binding site such as BINDING SITE I, BINDING SITE II or BINDING SITE III. Table 2 illustrates the complementarity of the nucleotide sequences of KIAA1028 BINDING SITE, designated SEQ ID:3546, to the nucleotide sequence of VGAM144 RNA, herein designated VGAM RNA, also designated SEQ ID:479.

Another function of VGAM144 is therefore inhibition of KIAA1028 (Accession XM_{—}166324). Accordingly, utilities of VGAM144 include diagnosis, prevention and treatment of diseases and clinical conditions associated with KIAA1028. PC2 (positive cofactor 2, multiprotein complex) Glutamine/Q-rich-associated Protein (PCQAP, Accession NM_{—}015889) is another VGAM144 host target gene. PCQAP BINDING SITE is HOST TARGET binding site found in the 3′ untranslated region of mRNA encoded by PCQAP, corresponding to a HOST TARGET binding site such as BINDING SITE I, BINDING SITE II or BINDING SITE III. Table 2 illustrates the complementarity of the nucleotide sequences of PCQAP BINDING SITE, designated SEQ ID: 1649, to the nucleotide sequence of VGAM144 RNA, herein designated VGAM RNA, also designated SEQ ID:479.

Another function of VGAM144 is therefore inhibition of PC2 (positive cofactor 2, multiprotein complex) Glutamine/Q-rich-associated Protein (PCQAP, Accession NM_{—}015889). Accordingly, utilities of VGAM144 include diagnosis, prevention and treatment of diseases and clinical conditions associated with PCQAP. Protein Kinase, Lysine Deficient 2 (PRKWNK2, Accession XM_{—}117531) is another VGAM144 host target gene. PRKWNK2 BINDING SITE is HOST TARGET binding site found in the 3′ untranslated region of mRNA encoded by PRKWNK2, corresponding to a HOST TARGET binding site such as BINDING SITE I, BINDING SITE II or BINDING SITE III. Table 2 illustrates the complementarity of the nucleotide sequences of PRKWNK2 BINDING SITE, designated SEQ ID:3488, to the nucleotide sequence of VGAM144 RNA, herein designated VGAM RNA, also designated SEQ ID:479.

Another function of VGAM144 is therefore inhibition of Protein Kinase, Lysine Deficient 2 (PRKWNK2, Accession XM_{—}117531). Accordingly, utilities of VGAM144 include diagnosis, prevention and treatment of diseases and clinical conditions associated with PRKWNK2. RCD-8 (Accession NM_{—}014329) is another VGAM144 host target gene. RCD-8 BINDING SITE is HOST TARGET binding site found in the 3′ untranslated region of mRNA encoded by RCD-8, corresponding to a HOST TARGET binding site such as BINDING SITE I, BINDING SITE II or BINDING SITE III. Table 2 illustrates the complementarity of the nucleotide sequences of RCD-8 BINDING SITE, designated SEQ ID:1491, to the nucleotide sequence of VGAM144 RNA, herein designated VGAM RNA, also designated SEQ ID:479.

Another function of VGAM144 is therefore inhibition of RCD-8 (Accession NM_{—}014329). Accordingly, utilities of VGAM144 include diagnosis, prevention and treatment of diseases and clinical conditions associated with RCD-8. SMOC2 (Accession XM_{—}051452) is another VGAM144 host target gene. SMOC2 BINDING SITE is HOST TARGET binding site found in the 3′ untranslated region of mRNA encoded by SMOC2, corresponding to a HOST TARGET binding site such as BINDING SITE I, BINDING SITE II or BINDING SITE III. Table 2 illustrates the complementarity of the nucleotide sequences of SMOC2 BINDING SITE, designated SEQ ID:2945, to the nucleotide sequence of VGAM144 RNA, herein designated VGAM RNA, also designated SEQ ID:479.

Another function of VGAM144 is therefore inhibition of SMOC2 (Accession XM_{—}051452). Accordingly, utilities of VGAM144 include diagnosis, prevention and treatment of diseases and clinical conditions associated with SMOC2. Synaptojanin 2 (SYNJ2, Accession XM_{—}029746) is another VGAM144 host target gene. SYNJ2 BINDING SITE is HOST TARGET binding site found in the 3′ untranslated region of mRNA encoded by SYNJ2, corresponding to a HOST TARGET binding site such as BINDING SITE I, BINDING SITE II or BINDING SITE III. Table 2 illustrates the complementarity of the nucleotide sequences of SYNJ2 BINDING SITE, designated SEQ ID:2605, to the nucleotide sequence of VGAM144 RNA, herein designated VGAM RNA, also designated SEQ ID:479.

Another function of VGAM144 is therefore inhibition of Synaptojanin 2 (SYNJ2, Accession XM_{—}029746). Accordingly, utilities of VGAM144 include diagnosis, prevention and treatment of diseases and clinical conditions associated with SYNJ2. TRABID (Accession XM_{—}043669) is another VGAM144 host target gene. TRABID BINDING SITE is HOST TARGET binding site found in the 3′ untranslated region of mRNA encoded by TRABID, corresponding to a HOST TARGET binding site such as BINDING SITE I, BINDING SITE II or BINDING SITE III. Table 2 illustrates the complementarity of the nucleotide sequences of TRABID BINDING SITE, designated SEQ ID:2823, to the nucleotide sequence of VGAM144 RNA, herein designated VGAM RNA, also designated SEQ ID:479.

Another function of VGAM144 is therefore inhibition of TRABID (Accession XM_{—}043669). Accordingly, utilities of VGAM144 include diagnosis, prevention and treatment of diseases and clinical conditions associated with TRABID. Testis Specific, 14 (TSGA14, Accession NM_{—}018718) is another VGAM144 host target gene. TSGA14 BINDING SITE is HOST TARGET binding site found in the 3′ untranslated region of mRNA encoded by TSGA14, corresponding to a HOST TARGET binding site such as BINDING SITE I, BINDING SITE II or BINDING SITE III. Table 2 illustrates the complementarity of the nucleotide sequences of TSGA14 BINDING SITE, designated SEQ ID:1861, to the nucleotide sequence of VGAM144 RNA, herein designated VGAM RNA, also designated SEQ ID:479.

Another function of VGAM144 is therefore inhibition of Testis Specific, 14 (TSGA14, Accession NM_{—}018718). Accordingly, utilities of VGAM144 include diagnosis, prevention and treatment of diseases and clinical conditions associated with TSGA14. LOC197201 (Accession XM_{—}113839) is another VGAM144 host target gene. LOC197201 BINDING SITE is HOST TARGET binding site found in the 3′ untranslated region of mRNA encoded by LOC197201, corresponding to a HOST TARGET binding site such as BINDING SITE I, BINDING SITE II or BINDING SITE III. Table 2 illustrates the complementarity of the nucleotide sequences of LOC197201 BINDING SITE, designated SEQ ID:3421, to the nucleotide sequence of VGAM144 RNA, herein designated VGAM RNA, also designated SEQ ID:479.

Another function of VGAM144 is therefore inhibition of LOC197201 (Accession XM_{—}113839). Accordingly, utilities of VGAM144 include diagnosis, prevention and treatment of diseases and clinical conditions associated with LOC197201. LOC219401 (Accession XM_{—}166706) is another VGAM144 host target gene. LOC219401 BINDING SITE is HOST TARGET binding site found in the 3′ untranslated region of mRNA encoded by LOC219401, corresponding to a HOST TARGET binding site such as BINDING SITE I, BINDING SITE II or BINDING SITE III. Table 2 illustrates the complementarity of the nucleotide sequences of LOC219401 BINDING SITE, designated SEQ ID:3579, to the nucleotide sequence of VGAM144 RNA, herein designated VGAM RNA, also designated SEQ ID:479.

Another function of VGAM144 is therefore inhibition of LOC219401 (Accession XM_{—}166706). Accordingly, utilities of VGAM144 include diagnosis, prevention and treatment of diseases and clinical conditions associated with LOC219401. FIG. 1 further provides a conceptual description of a novel bioinformatically detected viral gene of the present invention, referred to here as Viral Genomic Address Messenger 145 (VGAM145) viral gene, which modulates expression of respective host target genes thereof, the function and utility of which host target genes is known in the art.

VGAM145 is a novel bioinformatically detected regulatory, non protein coding, viral micro RNA (miRNA) gene. The method by which VGAM145 was detected is described hereinabove with reference to FIGS. 1-8.

VGAM145 gene, herein designated VGAM GENE, is a viral gene contained in the genome of Vaccinia Virus. VGAM145 host target gene, herein designated VGAM HOST TARGET GENE, is a human gene contained in the human genome.

VGAM145 gene encodes a VGAM145 precursor RNA, herein designated VGAM PRECURSOR RNA. Similar to other miRNA genes, and unlike most ordinary genes, VGAM145 precursor RNA does not encode a protein. A nucleotide sequence identical or highly similar to the nucleotide sequence of VGAM145 precursor RNA is designated SEQ ID:131, and is provided hereinbelow with reference to the sequence listing part. Nucleotide sequence SEQ ID:131 is located at position 67126 relative to the genome of Vaccinia Virus.

VGAM145 precursor RNA folds onto itself, forming VGAM145 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM145 folded precursor RNA into VGAM145 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM145 RNA is designated SEQ ID:480, and is provided hereinbelow with reference to the sequence listing part.

VGAM145 host target gene, herein designated VGAM HOST TARGET GENE, encodes a corresponding messenger RNA, VGAM145 host target RNA, herein designated VGAM HOST TARGET RNA. VGAM145 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM145 RNA, herein designated VGAM RNA, binds complementarily to one or more host target binding sites located in untranslated regions of VGAM145 host target RNA, herein designated VGAM HOST TARGET RNA. This complementary binding is due to the fact that the nucleotide sequence of VGAM145 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM145 RNA, herein designated VGAM RNA, may have a different number of host target binding sites in untranslated regions of a VGAM145 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM145 RNA, herein designated VGAM RNA, to host target binding sites on VGAM145 host target RNA, herein designated VGAM HOST TARGET RNA, such as BINDING SITE I, BINDING SITE II and BINDING SITE III, inhibits translation of VGAM145 host target RNA into VGAM145 host target protein, herein designated VGAM HOST TARGET PROTEIN. VGAM host target protein is therefore outlined by a broken line.

It is appreciated that VGAM145 host target gene, herein designated VGAM HOST TARGET GENE, in fact represents a plurality of VGAM145 host target genes. The mRNA of each one of this plurality of VGAM145 host target genes comprises one or more host target binding sites, each having a nucleotide sequence which is at least partly complementary to VGAM145 RNA, herein designated VGAM RNA, and which when bound by VGAM145 RNA causes inhibition of translation of respective one or more VGAM145 host target proteins.

It is further appreciated by one skilled in the art that the mode of translational inhibition illustrated by FIG. 1 with specific reference to translational inhibition exerted by VGAM145 gene, herein designated VGAM GENE, on one or more VGAM145 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM145 is inhibition of expression of host target genes, as part of a novel viral mechanism of attacking a host. Accordingly, utilities of VGAM145 include diagnosis, prevention and treatment of viral infection by Vaccinia Virus. Specific functions, and accordingly utilities, of VGAM145 correlate with, and may be deduced from, the identity of the host target genes which VGAM145 binds and inhibits, and the function of these host target genes, as elaborated hereinbelow.

Nucleotide sequences of the VGAM145 precursor RNA, herein designated VGAM PRECURSOR RNA, and of the ‘diced’ VGAM145 RNA, herein designated VGAM RNA, and a schematic representation of the secondary folding of VGAM145 folded precursor RNA, herein designated VGAM FOLDED PRECURSOR RNA, of VGAM145 are further described hereinbelow with reference to Table 1.

Nucleotide sequences of host target binding sites, such as BINDING SITE-I, BINDING SITE-II and BINDING SITE-III of FIG. 1, found on VGAM145 host target RNA, and schematic representation of the complementarity of each of these host target binding sites to VGAM145 RNA, herein designated VGAM RNA, are described hereinbelow with reference to Table 2.

As mentioned hereinabove with reference to FIG. 1, a function of VGAM145 gene, herein designated VGAM is inhibition of expression of VGAM145 target genes. It is appreciated that specific functions, and accordingly utilities, of VGAM145 correlate with, and may be deduced from, the identity of the target genes which VGAM145 binds and inhibits, and the function of these target genes, as elaborated hereinbelow.

Inhibin, Beta A (activin A, activin AB alpha polypeptide) (INHBA, Accession NM_{—}002192) is a VGAM145 host target gene. INHBA BINDING SITE is HOST TARGET binding site found in the 3′ untranslated region of mRNA encoded by INHBA, corresponding to a HOST TARGET binding site such as BINDING SITE I, BINDING SITE II or BINDING SITE III. Table 2 illustrates the complementarity of the nucleotide sequences of INHBA BINDING SITE, designated SEQ ID:904, to the nucleotide sequence of VGAM145 RNA, herein designated VGAM RNA, also designated SEQ ID:480.

A function of VGAM145 is therefore inhibition of Inhibin, Beta A (activin A, activin AB alpha polypeptide) (INHBA, Accession NM_{—}002192), a gene which inhibit respectively the secretion of follitropin by the pituitary gland. Accordingly, utilities of VGAM145 include diagnosis, prevention and treatment of diseases and clinical conditions associated with INHBA. The function of INHBA has been established by previous studies. From the culture fluid of a human transformed cell line (THB-1) stimulated by phorbol 12-myristate 13-acetate, Murata et al. (1988) isolated a protein that exhibited potent differentiation-inducing activity toward mouse Friend erythroleukemia cells and human K-562 cells. Designated erythroid differentiation factor (EDF), the protein is a homodimer with a molecular weight of 25,000. Surprisingly, the sequence of EDF mRNA was found to be identical to that of the beta-A subunit of inhibin. Southern blot analysis indicated that only 1 gene for EDF/inhibin beta-A exists in the human genome. The follicle-stimulating hormone (FSH)-releasing protein (FRP) subunit is likewise identical in structure to the beta-A subunit of inhibin. Lumpkin et al. (1987) purified from sheep hypothalamus a fraction (presumably a peptide) that had selective FSH-releasing properties. They demonstrated dissimilarity of the purified factor from luteinizing hormone-releasing hormone (OMIM Ref. No. 152760). You and Kruse (2002) studied corneal myofibroblast differentiation and signal transduction induced by the transforming growth factor-beta (TGFB) family members activin A and bone morphogenetic protein-7 (BMP7; 112267). They found that activin A induced phosphorylation of SMAD2 (OMIM Ref. No. 601366), and BMP7 induced SMAD1 (OMIM Ref. No. 601595), both of which were inhibited by follistatin (OMIM Ref. No. 136470). Transfection with antisense SMAD2/SMAD3 (OMIM Ref. No. 603109) prevented activin-induced expression and accumulation of alpha-smooth muscle actin. The authors concluded that TGFB proteins have different functions in the cornea. Activin A and TGFB1, but not BMP7, are regulators of keratocyte differentiation and might play a role during myofibroblast transdifferentiation. SMAD2/SMAD3 signal transduction appeared to be important in the regulation of muscle-specific genes. Animal model experiments lend further support to the function of INHBA. The activins, dimers of beta-A or beta-B subunits encoded by the genes Inhba and Inhbb, respectively, are TGF-beta super family members that have roles in reproduction and development. Whereas mice homozygous for the Inhba-null allele demonstrate disruption of whisker, palate, and tooth development leading to neonatal lethality, homozygous Inhbb-null mice are viable, fertile, and have eye defects. To determine if these phenotypes were due to spatiotemporal expression differences of the ligands or disruption of specific ligand-receptor interactions, Brown et al. (2000) replaced the region of Inhba encoding the mature protein with Inhbb, creating the allele designated Inhba(BK). Although the craniofacial phenotypes of the Inhba-null mutation were rescued by the Inhba(BK) allele, somatic, testicular, genital, and hair growth were grossly affected and influenced by the dosage and bioactivity of the allele. Thus, Brown et al. (2000) concluded that functional compensation within the TGF-beta super family can occur if the replacement gene is expressed appropriately. The novel phenotypes in these mice further illustrate the usefulness of insertion strategies for defining protein function. The structural organization of the testes of adult Inhba(BK/BK) mice was normal; however, the differentiation of the seminiferous tubules of Inhba(BK/−) mice was delayed. The testicular volumes of both Inhba(BK/BK) and Inhba(BK/−) mice were less than those of controls, and the dosage of the Inhba(BK) allele correlated positively with testicular size. Inhba(+/BK) males had normal onset of fertility, whereas Inhba(BK/BK) males had delayed onset of fertility similar to Acvr2 (OMIM Ref. No. 102581) −/− mice. Only 1 in 6 Inhba(BK/BK) females produced litters, whereas Inhba(+/BK) females were normally fertile. The ovaries of Inhba(BK/−) mice were smaller and contained fewer large preantral follicles than those of controls. Inhba(BK/BK) and Inhba(BK/−) mice were identified by their smaller size, slower hair growth, the rough appearance of their fur, and sunken eyes. Approximately 50% of Inhba(BK/BK) mice died by 26 weeks, whereas Inhba(BK/−) mice invariably became cachectic and died between 3 and 4 weeks. The summary of phenotypic findings of Inhba(BK/−) mice includes short whiskers, normal tooth development, no cleft palate, symmetric growth deficiency (OMIM Ref. No. severe), enlargement of external genitalia, hypogonadism (OMIM Ref. No. severe), delayed hair growth (moderate), hypoglycemia (mild), decreased life expectancy (OMIM Ref. No. severe), and anemia

It is appreciated that the abovementioned animal model for INHBA is acknowledged by those skilled in the art as a scientifically valid animal model, as can be further appreciated from the publications sited hereinbelow.

Full details of the abovementioned studies are described in the following publications, the disclosure of which are hereby incorporated by reference:
Brown, C. W.; Houston-Hawkins, D. E.; Woodruff, T. K.; Matzuk, M. M.: Insertion of Inhbb into the Inhba locus rescues the Inhba-null phenotype and reveals new activin functions. Nature Genet. 25: 453-457, 2000.; and
Murata, M.; Eto, Y.; Shibai, H.; Sakai, M.; Muramatsu, M.: Erythroid differentiation factor is encoded by the same mRNA as that of the inhibin beta-A chain. Proc. Nat. Acad. Sci. 85: 2.

Further studies establishing the function and utilities of INHBA are found in John Hopkins OMIM database record ID 147290, and in sited publications numbered 1190, 119 and 1193-1197 listed in the bibliography section hereinbelow, which are also hereby incorporated by reference. Thromboxane A Synthase 1 (platelet, cytochrome P450, sub family V) (TBXAS1, Accession NM_{—}030984) is another VGAM145 host target gene. TBXAS1 BINDING SITE1 and TBXAS1 BINDING SITE2 are HOST TARGET binding sites found in untranslated regions of mRNA encoded by TBXAS1, corresponding to HOST TARGET binding sites such as BINDING SITE I, BINDING SITE II or BINDING SITE III. Table 2 illustrates the complementarity of the nucleotide sequences of TBXAS1 BINDING SITE1 and TBXAS1 BINDING SITE2, designated SEQ ID:2182 and SEQ ID:798 respectively, to the nucleotide sequence of VGAM145 RNA, herein designated VGAM RNA, also designated SEQ ID:480.

Another function of VGAM145 is therefore inhibition of Thromboxane A Synthase 1 (platelet, cytochrome P450, sub family V) (TBXAS1, Accession NM_{—}030984). Accordingly, utilities of VGAM145 include diagnosis, prevention and treatment of diseases and clinical conditions associated with TBXAS1. LOC197342 (Accession XM_{—}113869) is another VGAM145 host target gene. LOC197342 BINDING SITE is HOST TARGET binding site found in the 3′ untranslated region of mRNA encoded by LOC197342, corresponding to a HOST TARGET binding site such as BINDING SITE I, BINDING SITE II or BINDING SITE III. Table 2 illustrates the complementarity of the nucleotide sequences of LOC197342 BINDING SITE, designated SEQ ID:3424, to the nucleotide sequence of VGAM145 RNA, herein designated VGAM RNA, also designated SEQ ID:480.

Another function of VGAM145 is therefore inhibition of LOC197342 (Accession XM_{—}113869). Accordingly, utilities of VGAM145 include diagnosis, prevention and treatment of diseases and clinical conditions associated with LOC197342. LOC91752 (Accession XM_{—}040403) is another VGAM145 host target gene. LOC91752 BINDING SITE is HOST TARGET binding site found in the 5′ untranslated region of mRNA encoded by LOC91752, corresponding to a HOST TARGET binding site such as BINDING SITE I, BINDING SITE II or BINDING SITE III. Table 2 illustrates the complementarity of the nucleotide sequences of LOC91752 BINDING SITE, designated SEQ ID:2779, to the nucleotide sequence of VGAM145 RNA, herein designated VGAM RNA, also designated SEQ ID:480.

Another function of VGAM145 is therefore inhibition of LOC91752 (Accession XM_{—}040403). Accordingly, utilities of VGAM145 include diagnosis, prevention and treatment of diseases and clinical conditions associated with LOC91752. FIG. 1 further provides a conceptual description of a novel bioinformatically detected viral gene of the present invention, referred to here as Viral Genomic Address Messenger 146 (VGAM146) viral gene, which modulates expression of respective host target genes thereof, the function and utility of which host target genes is known in the art.

VGAM146 is a novel bioinformatically detected regulatory, non protein coding, viral micro RNA (miRNA) gene. The method by which VGAM146 was detected is described hereinabove with reference to FIGS. 1-8.

VGAM146 gene, herein designated VGAM GENE, is a viral gene contained in the genome of Vaccinia Virus. VGAM146 host target gene, herein designated VGAM HOST TARGET GENE, is a human gene contained in the human genome.

VGAM146 gene encodes a VGAM146 precursor RNA, herein designated VGAM PRECURSOR RNA. Similar to other miRNA genes, and unlike most ordinary genes, VGAM146 precursor RNA does not encode a protein. A nucleotide sequence identical or highly similar to the nucleotide sequence of VGAM146 precursor RNA is designated SEQ ID:132, and is provided hereinbelow with reference to the sequence listing part. Nucleotide sequence SEQ ID:132 is located at position 69412 relative to the genome of Vaccinia Virus.

VGAM146 precursor RNA folds onto itself, forming VGAM146 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM146 folded precursor RNA into VGAM146 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM146 RNA is designated SEQ ID:481, and is provided hereinbelow with reference to the sequence listing part.

VGAM146 host target gene, herein designated VGAM HOST TARGET GENE, encodes a corresponding messenger RNA, VGAM146 host target RNA, herein designated VGAM HOST TARGET RNA. VGAM146 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM146 RNA, herein designated VGAM RNA, binds complementarily to one or more host target binding sites located in untranslated regions of VGAM146 host target RNA, herein designated VGAM HOST TARGET RNA. This complementary binding is due to the fact that the nucleotide sequence of VGAM146 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM146 RNA, herein designated VGAM RNA, may have a different number of host target binding sites in untranslated regions of a VGAM146 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM146 RNA, herein designated VGAM RNA, to host target binding sites on VGAM146 host target RNA, herein designated VGAM HOST TARGET RNA, such as BINDING SITE I, BINDING SITE II and BINDING SITE III, inhibits translation of VGAM146 host target RNA into VGAM146 host target protein, herein designated VGAM HOST TARGET PROTEIN. VGAM host target protein is therefore outlined by a broken line.

It is appreciated that VGAM146 host target gene, herein designated VGAM HOST TARGET GENE, in fact represents a plurality of VGAM146 host target genes. The mRNA of each one of this plurality of VGAM146 host target genes comprises one or more host target binding sites, each having a nucleotide sequence which is at least partly complementary to VGAM146 RNA, herein designated VGAM RNA, and which when bound by VGAM146 RNA causes inhibition of translation of respective one or more VGAM146 host target proteins.

It is further appreciated by one skilled in the art that the mode of translational inhibition illustrated by FIG. 1 with specific reference to translational inhibition exerted by VGAM146 gene, herein designated VGAM GENE, on one or more VGAM146 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM146 is inhibition of expression of host target genes, as part of a novel viral mechanism of attacking a host. Accordingly, utilities of VGAM146 include diagnosis, prevention and treatment of viral infection by Vaccinia Virus. Specific functions, and accordingly utilities, of VGAM146 correlate with, and may be deduced from, the identity of the host target genes which VGAM146 binds and inhibits, and the function of these host target genes, as elaborated hereinbelow.

Nucleotide sequences of the VGAM146 precursor RNA, herein designated VGAM PRECURSOR RNA, and of the ‘diced’ VGAM146 RNA, herein designated VGAM RNA, and a schematic representation of the secondary folding of VGAM146 folded precursor RNA, herein designated VGAM FOLDED PRECURSOR RNA, of VGAM146 are further described hereinbelow with reference to Table 1.

Nucleotide sequences of host target binding sites, such as BINDING SITE-I, BINDING SITE-II and BINDING SITE-III of FIG. 1, found on VGAM146 host target RNA, and schematic representation of the complementarity of each of these host target binding sites to VGAM146 RNA, herein designated VGAM RNA, are described hereinbelow with reference to Table 2.

As mentioned hereinabove with reference to FIG. 1, a function of VGAM146 gene, herein designated VGAM is inhibition of expression of VGAM146 target genes. It is appreciated that specific functions, and accordingly utilities, of VGAM146 correlate with, and may be deduced from, the identity of the target genes which VGAM146 binds and inhibits, and the function of these target genes, as elaborated hereinbelow.

FLJ11838 (Accession NM_{—}024664) is a VGAM146 host target gene. FLJ11838 BINDING SITE is HOST TARGET binding site found in the 3′ untranslated region of mRNA encoded by FLJ11838, corresponding to a HOST TARGET binding site such as BINDING SITE I, BINDING SITE II or BINDING SITE III. Table 2 illustrates the complementarity of the nucleotide sequences of FLJ11838 BINDING SITE, designated SEQ ID:2078, to the nucleotide sequence of VGAM146 RNA, herein designated VGAM RNA, also designated SEQ ID:481.

A function of VGAM146 is therefore inhibition of FLJ11838 (Accession NM_{—}024664). Accordingly, utilities of VGAM146 include diagnosis, prevention and treatment of diseases and clinical conditions associated with FLJ11838. FIG. 1 further provides a conceptual description of a novel bioinformatically detected viral gene of the present invention, referred to here as Viral Genomic Address Messenger 147 (VGAM147) viral gene, which modulates expression of respective host target genes thereof, the function and utility of which host target genes is known in the art.

VGAM147 is a novel bioinformatically detected regulatory, non protein coding, viral micro RNA (miRNA) gene. The method by which VGAM147 was detected is described hereinabove with reference to FIGS. 1-8.

VGAM147 gene, herein designated VGAM GENE, is a viral gene contained in the genome of Vaccinia Virus. VGAM147 host target gene, herein designated VGAM HOST TARGET GENE, is a human gene contained in the human genome.

VGAM147 gene encodes a VGAM147 precursor RNA, herein designated VGAM PRECURSOR RNA. Similar to other miRNA genes, and unlike most ordinary genes, VGAM147 precursor RNA does not encode a protein. A nucleotide sequence identical or highly similar to the nucleotide sequence of VGAM147 precursor RNA is designated SEQ ID:133, and is provided hereinbelow with reference to the sequence listing part. Nucleotide sequence SEQ ID:133 is located at position 66394 relative to the genome of Vaccinia Virus.

VGAM147 precursor RNA folds onto itself, forming VGAM147 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM147 folded precursor RNA into VGAM147 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM147 RNA is designated SEQ ID:482, and is provided hereinbelow with reference to the sequence listing part.

VGAM147 host target gene, herein designated VGAM HOST TARGET GENE, encodes a corresponding messenger RNA, VGAM147 host target RNA, herein designated VGAM HOST TARGET RNA. VGAM147 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM147 RNA, herein designated VGAM RNA, binds complementarily to one or more host target binding sites located in untranslated regions of VGAM147 host target RNA, herein designated VGAM HOST TARGET RNA. This complementary binding is due to the fact that the nucleotide sequence of VGAM147 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM147 RNA, herein designated VGAM RNA, may have a different number of host target binding sites in untranslated regions of a VGAM147 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM147 RNA, herein designated VGAM RNA, to host target binding sites on VGAM147 host target RNA, herein designated VGAM HOST TARGET RNA, such as BINDING SITE I, BINDING SITE II and BINDING SITE III, inhibits translation of VGAM147 host target RNA into VGAM147 host target protein, herein designated VGAM HOST TARGET PROTEIN. VGAM host target protein is therefore outlined by a broken line.

It is appreciated that VGAM147 host target gene, herein designated VGAM HOST TARGET GENE, in fact represents a plurality of VGAM147 host target genes. The mRNA of each one of this plurality of VGAM147 host target genes comprises one or more host target binding sites, each having a nucleotide sequence which is at least partly complementary to VGAM147 RNA, herein designated VGAM RNA, and which when bound by VGAM147 RNA causes inhibition of translation of respective one or more VGAM147 host target proteins.

It is further appreciated by one skilled in the art that the mode of translational inhibition illustrated by FIG. 1 with specific reference to translational inhibition exerted by VGAM147 gene, herein designated VGAM GENE, on one or more VGAM147 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM147 is inhibition of expression of host target genes, as part of a novel viral mechanism of attacking a host. Accordingly, utilities of VGAM147 include diagnosis, prevention and treatment of viral infection by Vaccinia Virus. Specific functions, and accordingly utilities, of VGAM147 correlate with, and may be deduced from, the identity of the host target genes which VGAM147 binds and inhibits, and the function of these host target genes, as elaborated hereinbelow.

Nucleotide sequences of the VGAM147 precursor RNA, herein designated VGAM PRECURSOR RNA, and of the ‘diced’ VGAM147 RNA, herein designated VGAM RNA, and a schematic representation of the secondary folding of VGAM147 folded precursor RNA, herein designated VGAM FOLDED PRECURSOR RNA, of VGAM147 are further described hereinbelow with reference to Table 1.

Nucleotide sequences of host target binding sites, such as BINDING SITE-I, BINDING SITE-II and BINDING SITE-III of FIG. 1, found on VGAM147 host target RNA, and schematic representation of the complementarity of each of these host target binding sites to VGAM147 RNA, herein designated VGAM RNA, are described hereinbelow with reference to Table 2.

As mentioned hereinabove with reference to FIG. 1, a function of VGAM147 gene, herein designated VGAM is inhibition of expression of VGAM147 target genes. It is appreciated that specific functions, and accordingly utilities, of VGAM147 correlate with, and may be deduced from, the identity of the target genes which VGAM147 binds and inhibits, and the function of these target genes, as elaborated hereinbelow.

ATP10C (Accession NM_{—}024490) is a VGAM147 host target gene. ATP10C BINDING SITE is HOST TARGET binding site found in the 3′ untranslated region of mRNA encoded by ATP10C, corresponding to a HOST TARGET binding site such as BINDING SITE I, BINDING SITE II or BINDING SITE III. Table 2 illustrates the complementarity of the nucleotide sequences of ATP10C BINDING SITE, designated SEQ ID:2062, to the nucleotide sequence of VGAM147 RNA, herein designated VGAM RNA, also designated SEQ ID:482.

A function of VGAM147 is therefore inhibition of ATP10C (Accession NM_{—}024490), a gene which is phosphorylated in their intermediate state, drives uphill transport of ions across membranes. Accordingly, utilities of VGAM147 include diagnosis, prevention and treatment of diseases and clinical conditions associated with ATP10C. The function of ATP10C has been established by previous studies. Meguro et al. (2001) reported that the ATP10C gene is maternally expressed, that it maps within the most common interval of deletion responsible for Angelman syndrome (AS; 105830) (15q11-q13), and that ATP10C expression is virtually absent from Angelman syndrome patients with imprinting mutations, as well as from patients with maternal deletions of 15q11-q13. Previously, although AS patients infrequently have mutations in the UBE3A gene (OMIM Ref. No. 601623), which encodes a ubiquitin ligase required for long-term synaptic potentiation (LTP), most cases were attributable to de novo maternal deletions of the critical 15q region. Herzing et al. (2001) reported that ATP10C maps within 200 kb distal to UBE3A and, like UBE3A, demonstrates imprinted, preferential maternal expression in human brain. They suggested that ATP10C is a candidate for chromosome 15-associated autism as well as the Angelman syndrome phenotype. Animal model experiments lend further support to the function of ATP10C. Dhar et al. (2000) reported that maternal inheritance of deletions of the mouse Atp10c gene resulted in increased body fat. The obese phenotype was consistently observed in the mouse model for Angelman syndrome with paternal uniparental disomy (Cattanach et al., 1997). Meguro et al. (2001) speculated that ATP10C may be an aminophospholipid translocase involved in phospholipid transport.

It is appreciated that the abovementioned animal model for ATP10C is acknowledged by those skilled in the art as a scientifically valid animal model, as can be further appreciated from the publications sited hereinbelow.

Full details of the abovementioned studies are described in the following publications, the disclosure of which are hereby incorporated by reference:
Meguro, M.; Kashiwagi, A.; Mitsuya, K.; Nakao, M.; Kondo, I.; Saitoh, S.; Oshimura, M.: A novel maternally expressed gene, ATP10C, encodes a putative aminophospholipid translocase associated with Angelman syndrome. Nature Genet. 28: 19-20, 2001.; and
Cattanach, B. M.; Barr, J. A.; Beechey, C. V.; Martin, J.; Noebels, J.; Jones, J.: A candidate model for Angelman syndrome in the mouse. Mammalian Genome 8: 472-478, 1997.

Further studies establishing the function and utilities of ATP10C are found in John Hopkins OMIM database record ID 605855, and in sited publications numbered 1535-1536, 2905, 156 and 1567 listed in the bibliography section hereinbelow, which are also hereby incorporated by reference. Caspase 10, Apoptosis-related Cysteine Protease (CASP10, Accession NM_{—}032976) is another VGAM147 host target gene. CASP10 BINDING SITE is HOST TARGET binding site found in the 3′ untranslated region of mRNA encoded by CASP10, corresponding to a HOST TARGET binding site such as BINDING SITE I, BINDING SITE II or BINDING SITE III. Table 2 illustrates the complementarity of the nucleotide sequences of CASP10 BINDING SITE, designated SEQ ID:2304, to the nucleotide sequence of VGAM147 RNA, herein designated VGAM RNA, also designated SEQ ID:482.

Another function of VGAM147 is therefore inhibition of Caspase 10, Apoptosis-related Cysteine Protease (CASP10, Accession NM_{—}032976), a gene which is one aspartate-specific cysteine protease and important in death receptor signaling or other cellular processes. Accordingly, utilities of VGAM147 include diagnosis, prevention and treatment of diseases and clinical conditions associated with CASP10. The function of CASP10 has been established by previous studies. Wang et al. (2001) showed that caspase-10 can function independently of caspase-8 in initiating FAS- and tumor necrosis factor-related apoptosis-inducing ligand-receptor-mediated apoptosis. Moreover, FAS crosslinking in primary human T cells leads to the recruitment and activation of caspase-10. They showed that the death-effector domains of caspases 8 and 10 interact with the death-effector domain of FADD. Nonetheless, they found that caspases 8 and 10 may have different apoptosis substrates and therefore potentially distinct roles in death receptor signaling or other cellular processes. By a candidate gene mutation search strategy, Wang et al. (1999) identified independent missense mutations in the CASP10 gene in 2 kindreds with type II autoimmune lymphoproliferative syndrome (ALPS2; 603909) characterized by abnormal lymphocyte and dendritic cell homeostasis and immune regulatory defects. The mutations (601762.0001 and 601762.0002) resulted in amino acid substitutions that decreased caspase activity and interfered with death receptor-induced apoptosis, particularly that stimulated by Fas ligand (OMIM Ref. No. 134638) and TRAIL (OMIM Ref. No. 603598). These results provided evidence that inherited nonlethal caspase abnormalities cause pleiotropic apoptosis defects underlying autoimmunity in ALPS2. To explore the possibility that mutation in the CASP10 gene might be involved in the development of non-Hodgkin lymphoma (NHL; 605027), Shin et al. (2002) analyzed the entire coding region and all splice sites of the CASP10 gene for the detection of somatic mutations in 117 human NHLs. Seventeen NHLs (14.5%) had CASP10 mutations, of which 3 were identified in the coding regions of the prodomain, 11 in the p17 large protease subunit, and 3 in the p12 small protease subunit. There were 2 frameshift mutations and 1 nonsense mutation; the remaining 14 were missense mutations. Shin et al. (2002) expressed the tumor-derived CASP10 mutants in 293 cells and found that apoptosis was suppressed. These data suggested that the inactivating mutations of the CASP10 gene may lead to the loss of its apoptotic function and contribute to the pathogenesis of some human NHLs.

Full details of the abovementioned studies are described in the following publications, the disclosure of which are hereby incorporated by reference:
Wang, J.; Chun, H. J.; Wong, W.; Spencer, D. M.; Lenardo, M. J.: Caspase-10 is an initiator caspase in death receptor signaling. Proc. Nat. Acad. Sci. 98: 13884-13888, 2001.; and
Wang, J.; Zheng, L.; Lobito, A.; Chan, F. K.; Dale, J.; Sneller, M.; Yao, X.; Puck, J. M.; Straus, S. E.; Lenardo, M. J.: Inherited human caspase 10 mutations underlie defective lympho.

Further studies establishing the function and utilities of CASP10 are found in John Hopkins OMIM database record ID 601762, and in sited publications numbered 1628-1561 listed in the bibliography section hereinbelow, which are also hereby incorporated by reference. Zinc Finger Protein 36 (KOX 18) (ZNF36, Accession XM_{—}168302) is another VGAM147 host target gene. ZNF36 BINDING SITE is HOST TARGET binding site found in the 3′ untranslated region of mRNA encoded by ZNF36, corresponding to a HOST TARGET binding site such as BINDING SITE I, BINDING SITE II or BINDING SITE III. Table 2 illustrates the complementarity of the nucleotide sequences of ZNF36 BINDING SITE, designated SEQ ID:3627, to the nucleotide sequence of VGAM147 RNA, herein designated VGAM RNA, also designated SEQ ID:482.

Another function of VGAM147 is therefore inhibition of Zinc Finger Protein 36 (KOX 18) (ZNF36, Accession XM_{—}168302), a gene which may be involved in transcriptional regulation. Accordingly, utilities of VGAM147 include diagnosis, prevention and treatment of diseases and clinical conditions associated with ZNF36. The function of ZNF36 and its association with various diseases and clinical conditions, has been established by previous studies, as described hereinabove with reference to VGAM137. DORFIN (Accession NM_{—}015435) is another VGAM147 host target gene. DORFIN BINDING SITE is HOST TARGET binding site found in the 3′ untranslated region of mRNA encoded by DORFIN, corresponding to a HOST TARGET binding site such as BINDING SITE I, BINDING SITE II or BINDING SITE III. Table 2 illustrates the complementarity of the nucleotide sequences of DORFIN BINDING SITE, designated SEQ ID: 1630, to the nucleotide sequence of VGAM147 RNA, herein designated VGAM RNA, also designated SEQ ID:482.

Another function of VGAM147 is therefore inhibition of DORFIN (Accession NM_{—}015435). Accordingly, utilities of VGAM147 include diagnosis, prevention and treatment of diseases and clinical conditions associated with DORFIN. FLJ21313 (Accession NM_{—}023927) is another VGAM147 host target gene. FLJ21313 BINDING SITE is HOST TARGET binding site found in the 3′ untranslated region of mRNA encoded by FLJ21313, corresponding to a HOST TARGET binding site such as BINDING SITE I, BINDING SITE II or BINDING SITE III. Table 2 illustrates the complementarity of the nucleotide sequences of FLJ21313 BINDING SITE, designated SEQ ID:2037, to the nucleotide sequence of VGAM147 RNA, herein designated VGAM RNA, also designated SEQ ID:482.

Another function of VGAM147 is therefore inhibition of FLJ21313 (Accession NM_{—}023927). Accordingly, utilities of VGAM147 include diagnosis, prevention and treatment of diseases and clinical conditions associated with FLJ21313. KIAA1819 (Accession XM_{—}045716) is another VGAM147 host target gene. KIAA1819 BINDING SITE is HOST TARGET binding site found in the 5′ untranslated region of mRNA encoded by KIAA1819, corresponding to a HOST TARGET binding site such as BINDING SITE I, BINDING SITE II or BINDING SITE III. Table 2 illustrates the complementarity of the nucleotide sequences of KIAA1819 BINDING SITE, designated SEQ ID:2865, to the nucleotide sequence of VGAM147 RNA, herein designated VGAM RNA, also designated SEQ ID:482.

Another function of VGAM147 is therefore inhibition of KIAA1819 (Accession XM_{—}045716). Accordingly, utilities of VGAM147 include diagnosis, prevention and treatment of diseases and clinical conditions associated with KIAA1819. P37NB (Accession NM_{—}005824) is another VGAM147 host target gene. P37NB BINDING SITE is HOST TARGET binding site found in the 3′ untranslated region of mRNA encoded by P37NB, corresponding to a HOST TARGET binding site such as BINDING SITE I, BINDING SITE II or BINDING SITE III. Table 2 illustrates the complementarity of the nucleotide sequences of P37NB BINDING SITE, designated SEQ ID:1254, to the nucleotide sequence of VGAM147 RNA, herein designated VGAM RNA, also designated SEQ ID:482.

Another function of VGAM147 is therefore inhibition of P37NB (Accession NM_{—}005824). Accordingly, utilities of VGAM147 include diagnosis, prevention and treatment of diseases and clinical conditions associated with P37NB. RAP140 (Accession NM_{—}015224) is another VGAM147 host target gene. RAP140 BINDING SITE is HOST TARGET binding site found in the 5′ untranslated region of mRNA encoded by RAP140, corresponding to a HOST TARGET binding site such as BINDING SITE I, BINDING SITE II or BINDING SITE III. Table 2 illustrates the complementarity of the nucleotide sequences of RAP140 BINDING SITE, designated SEQ ID: 1613, to the nucleotide sequence of VGAM147 RNA, herein designated VGAM RNA, also designated SEQ ID:482.

Another function of VGAM147 is therefore inhibition of RAP140 (Accession NM_{—}015224). Accordingly, utilities of VGAM147 include diagnosis, prevention and treatment of diseases and clinical conditions associated with RAP140. LOC127002 (Accession XM_{—}059107) is another VGAM147 host target gene. LOC127002 BINDING SITE is HOST TARGET binding site found in the 3′ untranslated region of mRNA encoded by LOC127002, corresponding to a HOST TARGET binding site such as BINDING SITE I, BINDING SITE II or BINDING SITE III. Table 2 illustrates the complementarity of the nucleotide sequences of LOC127002 BINDING SITE, designated SEQ ID:3002, to the nucleotide sequence of VGAM147 RNA, herein designated VGAM RNA, also designated SEQ ID:482.

Another function of VGAM147 is therefore inhibition of LOC127002 (Accession XM_{—}059107). Accordingly, utilities of VGAM147 include diagnosis, prevention and treatment of diseases and clinical conditions associated with LOC127002. LOC132332 (Accession XM_{—}072306) is another VGAM147 host target gene. LOC132332 BINDING SITE is HOST TARGET binding site found in the 3′ untranslated region of mRNA encoded by LOC132332, corresponding to a HOST TARGET binding site such as BINDING SITE I, BINDING SITE II or BINDING SITE III. Table 2 illustrates the complementarity of the nucleotide sequences of LOC132332 BINDING SITE, designated SEQ ID:3046, to the nucleotide sequence of VGAM147 RNA, herein designated VGAM RNA, also designated SEQ ID:482.

Another function of VGAM147 is therefore inhibition of LOC132332 (Accession XM_{—}072306). Accordingly, utilities of VGAM147 include diagnosis, prevention and treatment of diseases and clinical conditions associated with LOC132332. LOC145624 (Accession XM_{—}096824) is another VGAM147 host target gene. LOC145624 BINDING SITE is HOST TARGET binding site found in the 5′ untranslated region of mRNA encoded by LOC145624, corresponding to a HOST TARGET binding site such as BINDING SITE I, BINDING SITE II or BINDING SITE III. Table 2 illustrates the complementarity of the nucleotide sequences of LOC145624 BINDING SITE, designated SEQ ID:3276, to the nucleotide sequence of VGAM147 RNA, herein designated VGAM RNA, also designated SEQ ID:482.

Another function of VGAM147 is therefore inhibition of LOC145624 (Accession XM_{—}096824). Accordingly, utilities of VGAM147 include diagnosis, prevention and treatment of diseases and clinical conditions associated with LOC145624. FIG. 1 further provides a conceptual description of a novel bioinformatically detected viral gene of the present invention, referred to here as Viral Genomic Address Messenger 148 (VGAM148) viral gene, which modulates expression of respective host target genes thereof, the function and utility of which host target genes is known in the art.

VGAM148 is a novel bioinformatically detected regulatory, non protein coding, viral micro RNA (miRNA) gene. The method by which VGAM148 was detected is described hereinabove with reference to FIGS. 1-8.

VGAM148 gene, herein designated VGAM GENE, is a viral gene contained in the genome of Vaccinia Virus. VGAM148 host target gene, herein designated VGAM HOST TARGET GENE, is a human gene contained in the human genome.

VGAM148 gene encodes a VGAM148 precursor RNA, herein designated VGAM PRECURSOR RNA. Similar to other miRNA genes, and unlike most ordinary genes, VGAM148 precursor RNA does not encode a protein. A nucleotide sequence identical or highly similar to the nucleotide sequence of VGAM148 precursor RNA is designated SEQ ID:134, and is provided hereinbelow with reference to the sequence listing part. Nucleotide sequence SEQ ID:134 is located at position 72012 relative to the genome of Vaccinia Virus.

VGAM148 precursor RNA folds onto itself, forming VGAM148 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM148 folded precursor RNA into VGAM148 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM148 RNA is designated SEQ ID:483, and is provided hereinbelow with reference to the sequence listing part.

VGAM148 host target gene, herein designated VGAM HOST TARGET GENE, encodes a corresponding messenger RNA, VGAM148 host target RNA, herein designated VGAM HOST TARGET RNA. VGAM148 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM148 RNA, herein designated VGAM RNA, binds complementarily to one or more host target binding sites located in untranslated regions of VGAM148 host target RNA, herein designated VGAM HOST TARGET RNA. This complementary binding is due to the fact that the nucleotide sequence of VGAM148 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM148 RNA, herein designated VGAM RNA, may have a different number of host target binding sites in untranslated regions of a VGAM148 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM148 RNA, herein designated VGAM RNA, to host target binding sites on VGAM148 host target RNA, herein designated VGAM HOST TARGET RNA, such as BINDING SITE I, BINDING SITE II and BINDING SITE III, inhibits translation of VGAM148 host target RNA into VGAM148 host target protein, herein designated VGAM HOST TARGET PROTEIN. VGAM host target protein is therefore outlined by a broken line.

It is appreciated that VGAM148 host target gene, herein designated VGAM HOST TARGET GENE, in fact represents a plurality of VGAM148 host target genes. The mRNA of each one of this plurality of VGAM148 host target genes comprises one or more host target binding sites, each having a nucleotide sequence which is at least partly complementary to VGAM148 RNA, herein designated VGAM RNA, and which when bound by VGAM148 RNA causes inhibition of translation of respective one or more VGAM148 host target proteins.

It is further appreciated by one skilled in the art that the mode of translational inhibition illustrated by FIG. 1 with specific reference to translational inhibition exerted by VGAM148 gene, herein designated VGAM GENE, on one or more VGAM148 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM148 is inhibition of expression of host target genes, as part of a novel viral mechanism of attacking a host. Accordingly, utilities of VGAM148 include diagnosis, prevention and treatment of viral infection by Vaccinia Virus. Specific functions, and accordingly utilities, of VGAM148 correlate with, and may be deduced from, the identity of the host target genes which VGAM148 binds and inhibits, and the function of these host target genes, as elaborated hereinbelow.

Nucleotide sequences of the VGAM148 precursor RNA, herein designated VGAM PRECURSOR RNA, and of the ‘diced’ VGAM148 RNA, herein designated VGAM RNA, and a schematic representation of the secondary folding of VGAM148 folded precursor RNA, herein designated VGAM FOLDED PRECURSOR RNA, of VGAM148 are further described hereinbelow with reference to Table 1.

Nucleotide sequences of host target binding sites, such as BINDING SITE-I, BINDING SITE-II and BINDING SITE-III of FIG. 1, found on VGAM148 host target RNA, and schematic representation of the complementarity of each of these host target binding sites to VGAM148 RNA, herein designated VGAM RNA, are described hereinbelow with reference to Table 2.

As mentioned hereinabove with reference to FIG. 1, a function of VGAM148 gene, herein designated VGAM is inhibition of expression of VGAM148 target genes. It is appreciated that specific functions, and accordingly utilities, of VGAM148 correlate with, and may be deduced from, the identity of the target genes which VGAM148 binds and inhibits, and the function of these target genes, as elaborated hereinbelow.

Nuclear Receptor Sub family 4, Group A, Member 2 (NR4A2, Accession NM_{—}006186) is a VGAM148 host target gene. NR4A2 BINDING SITE is HOST TARGET binding site found in the 3′ untranslated region of mRNA encoded by NR4A2, corresponding to a HOST TARGET binding site such as BINDING SITE I, BINDING SITE II or BINDING SITE III. Table 2 illustrates the complementarity of the nucleotide sequences of NR4A2 BINDING SITE, designated SEQ ID:1277, to the nucleotide sequence of VGAM148 RNA, herein designated VGAM RNA, also designated SEQ ID:483.

A function of VGAM148 is therefore inhibition of Nuclear Receptor Sub family 4, Group A, Member 2 (NR4A2, Accession NM_{—}006186), a gene which may be a general coactivator of transcription. Accordingly, utilities of VGAM148 include diagnosis, prevention and treatment of diseases and clinical conditions associated with NR4A2. The function of NR4A2 has been established by previous studies. Mages et al. (1994) analyzed early genetic response of human T cells following mitogenic activation and identified a gene encoding a member of the steroid/thyroid hormone family of receptors (see OMIM Ref. No. 190160). The receptor, called NOT (nuclear receptor of T cells) by them, has all of the structural features of steroid/thyroid hormone receptors but is rapidly and only very transiently expressed after cell activation. Short-lived NOT mRNA expression was observed in vivo exclusively in the brain. The C-terminal region of the predicted 598-amino acid NOT protein shares 71% similarity with NAK1/TR3 (OMIM Ref. No. 139139), a previously identified human orphan steroid receptor. The protein sequence is 99.5% similar to the murine Nurr1 gene, originally reported by Law et al. (1992) as a member of a nuclear receptor family that is highly expressed in brain and related to Nur77, an immediate-early response gene product. Animal model experiments lend further support to the function of NR4A2. Dopamine neurons of the substantia nigra and ventral tegmental area regulate movement and affective behavior and degenerate in Parkinson disease (OMIM Ref. No. 168600). Zetterstrom et al. (1997) showed that mouse Nurr1 is expressed in developing dopamine neurons before the appearance of known phenotypic markers for these cells. They also demonstrated that Nurr1 is an absolute requirement for development of midbrain dopamine neurons. Mice lacking the Nurr1 gene failed to generate brain dopaminergic neurons, were hypoactive, and died soon after birth. Nurr1 expression continued into adulthood, and brains of heterozygous animals, otherwise apparently healthy, contained reduced dopamine levels. The authors suggested that putative Nurr1 ligands may be useful treatment of Parkinson disease and other disorders of midbrain dopamine circuitry.

It is appreciated that the abovementioned animal model for NR4A2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Mages, H. W.; Rilke, O.; Bravo, R.; Senger, G.; Kroczek, R. A.: NOT, a human immediate-early response gene closely related to the steroid/thyroid hormone receptor NAK1/TR3. Molec. Endocr. 8: 1583-1591, 1994.; and
Zetterstrom, R. H.; Solomin, L.; Jansson, L.; Hoffer, B. J.; Olson, L.; Perlmann, T.: Dopamine neuron agenesis in Nurr1-deficient mice. Science 276: 248-250, 1997.

Further studies establishing the function and utilities of NR4A2 are found in John Hopkins OMIM database record ID 601828, and in sited publications numbered 1964-197 and 1111 listed in the bibliography section hereinbelow, which are also hereby incorporated by reference. Protein Kinase C, Nu (PRKCN, Accession NM_{—}005813) is another VGAM148 host target gene. PRKCN BINDING SITE is HOST TARGET binding site found in the 3′ untranslated region of mRNA encoded by PRKCN, corresponding to a HOST TARGET binding site such as BINDING SITE I, BINDING SITE II or BINDING SITE III. Table 2 illustrates the complementarity of the nucleotide sequences of PRKCN BINDING SITE, designated SEQ ID:1252, to the nucleotide sequence of VGAM148 RNA, herein designated VGAM RNA, also designated SEQ ID:483.

Another function of VGAM148 is therefore inhibition of Protein Kinase C, Nu (PRKCN, Accession NM_{—}005813). Accordingly, utilities of VGAM148 include diagnosis, prevention and treatment of diseases and clinical conditions associated with PRKCN. Basic, Immunoglobulin-like Variable Motif Containing (BIVM, Accession NM_{—}017693) is another VGAM148 host target gene. BIVM BINDING SITE is HOST TARGET binding site found in the 3′ untranslated region of mRNA encoded by BIVM, corresponding to a HOST TARGET binding site such as BINDING SITE I, BINDING SITE II or BINDING SITE III. Table 2 illustrates the complementarity of the nucleotide sequences of BIVM BINDING SITE, designated SEQ ID:1739, to the nucleotide sequence of VGAM148 RNA, herein designated VGAM RNA, also designated SEQ ID:483.

Another function of VGAM148 is therefore inhibition of Basic, Immunoglobulin-like Variable Motif Containing (BIVM, Accession NM_{—}017693). Accordingly, utilities of VGAM148 include diagnosis, prevention and treatment of diseases and clinical conditions associated with BIVM. DNA Cross-link Repair 1A (PSO2 homolog, S. cerevisiae) (DCLRE1A, Accession XM_{—}044815) is another VGAM148 host target gene. DCLRE1A BINDING SITE is HOST TARGET binding site found in the 3′ untranslated region of mRNA encoded by DCLRE1A, corresponding to a HOST TARGET binding site such as BINDING SITE I, BINDING SITE II or BINDING SITE III. Table 2 illustrates the complementarity of the nucleotide sequences of DCLRE1A BINDING SITE, designated SEQ ID:2843, to the nucleotide sequence of VGAM148 RNA, herein designated VGAM RNA, also designated SEQ ID:483.

Another function of VGAM148 is therefore inhibition of DNA Cross-link Repair 1A (PSO2 homolog, S. cerevisiae) (DCLRE1A, Accession XM_{—}044815). Accordingly, utilities of VGAM148 include diagnosis, prevention and treatment of diseases and clinical conditions associated with DCLRE1A. Early B-cell Factor 2 (EBF2, Accession NM_{—}022659) is another VGAM148 host target gene. EBF2 BINDING SITE is HOST TARGET binding site found in the 3′ untranslated region of mRNA encoded by EBF2, corresponding to a HOST TARGET binding site such as BINDING SITE I, BINDING SITE II or BINDING SITE III. Table 2 illustrates the complementarity of the nucleotide sequences of EBF2 BINDING SITE, designated SEQ ID:1995, to the nucleotide sequence of VGAM148 RNA, herein designated VGAM RNA, also designated SEQ ID:483.

Another function of VGAM148 is therefore inhibition of Early B-cell Factor 2 (EBF2, Accession NM_{—}022659). Accordingly, utilities of VGAM148 include diagnosis, prevention and treatment of diseases and clinical conditions associated with EBF2. FLJ10852 (Accession NM_{—}019028) is another VGAM148 host target gene. FLJ10852 BINDING SITE is HOST TARGET binding site found in the 3′ untranslated region of mRNA encoded by FLJ10852, corresponding to a HOST TARGET binding site such as BINDING SITE I, BINDING SITE II or BINDING SITE III. Table 2 illustrates the complementarity of the nucleotide sequences of FLJ10852 BINDING SITE, designated SEQ ID:1873, to the nucleotide sequence of VGAM148 RNA, herein designated VGAM RNA, also designated SEQ ID:483.

Another function of VGAM148 is therefore inhibition of FLJ10852 (Accession NM_{—}019028). Accordingly, utilities of VGAM148 include diagnosis, prevention and treatment of diseases and clinical conditions associated with FLJ10852. FLJ20220 (Accession NM_{—}017718) is another VGAM148 host target gene. FLJ20220 BINDING SITE is HOST TARGET binding site found in the 3′ untranslated region of mRNA encoded by FLJ20220, corresponding to a HOST TARGET binding site such as BINDING SITE I, BINDING SITE II or BINDING SITE III. Table 2 illustrates the complementarity of the nucleotide sequences of FLJ20220 BINDING SITE, designated SEQ ID:1742, to the nucleotide sequence of VGAM148 RNA, herein designated VGAM RNA, also designated SEQ ID:483.

Another function of VGAM148 is therefore inhibition of FLJ20220 (Accession NM_{—}017718). Accordingly, utilities of VGAM148 include diagnosis, prevention and treatment of diseases and clinical conditions associated with FLJ20220. FLJ21952 (Accession NM_{—}022494) is another VGAM148 host target gene. FLJ21952 BINDING SITE is HOST TARGET binding site found in the 3′ untranslated region of mRNA encoded by FLJ21952, corresponding to a HOST TARGET binding site such as BINDING SITE I, BINDING SITE II or BINDING SITE III. Table 2 illustrates the complementarity of the nucleotide sequences of FLJ21952 BINDING SITE, designated SEQ ID:1992, to the nucleotide sequence of VGAM148 RNA, herein designated VGAM RNA, also designated SEQ ID:483.

Another function of VGAM148 is therefore inhibition of FLJ21952 (Accession NM_{—}022494). Accordingly, utilities of VGAM148 include diagnosis, prevention and treatment of diseases and clinical conditions associated with FLJ21952. MGC10724 (Accession NM_{—}032720) is another VGAM148 host target gene. MGC10724 BINDING SITE is HOST TARGET binding site found in the 3′ untranslated region of mRNA encoded by MGC10724, corresponding to a HOST TARGET binding site such as BINDING SITE I, BINDING SITE II or BINDING SITE III. Table 2 illustrates the complementarity of the nucleotide sequences of MGC10724 BINDING SITE, designated SEQ ID:2274, to the nucleotide sequence of VGAM148 RNA, herein designated VGAM RNA, also designated SEQ ID:483.

Another function of VGAM148 is therefore inhibition of MGC10724 (Accession NM_{—}032720). Accordingly, utilities of VGAM148 include diagnosis, prevention and treatment of diseases and clinical conditions associated with MGC10724. MGC4238 (Accession NM_{—}032332) is another VGAM148 host target gene. MGC4238 BINDING SITE is HOST TARGET binding site found in the 3′ untranslated region of mRNA encoded by MGC4238, corresponding to a HOST TARGET binding site such as BINDING SITE I, BINDING SITE II or BINDING SITE III. Table 2 illustrates the complementarity of the nucleotide sequences of MGC4238 BINDING SITE, designated SEQ ID:2247, to the nucleotide sequence of VGAM148 RNA, herein designated VGAM RNA, also designated SEQ ID:483.

Another function of VGAM148 is therefore inhibition of MGC4238 (Accession NM_{—}032332). Accordingly, utilities of VGAM148 include diagnosis, prevention and treatment of diseases and clinical conditions associated with MGC4238. Rab11-FIP2 (Accession NM_{—}014904) is another VGAM148 host target gene. Rab11-FIP2 BINDING SITE is HOST TARGET binding site found in the 3′ untranslated region of mRNA encoded by Rab11-FIP2, corresponding to a HOST TARGET binding site such as BINDING SITE I, BINDING SITE II or BINDING SITE III. Table 2 illustrates the complementarity of the nucleotide sequences of Rab11-FIP2 BINDING SITE, designated SEQ ID:1581, to the nucleotide sequence of VGAM148 RNA, herein designated VGAM RNA, also designated SEQ ID:483.

Another function of VGAM148 is therefore inhibition of Rab11-FIP2 (Accession NM_{—}014904). Accordingly, utilities of VGAM148 include diagnosis, prevention and treatment of diseases and clinical conditions associated with Rab11-FIP2. LOC90768 (Accession XM_{—}033986) is another VGAM148 host target gene. LOC90768 BINDING SITE is HOST TARGET binding site found in the 3′ untranslated region of mRNA encoded by LOC90768, corresponding to a HOST TARGET binding site such as BINDING SITE I, BINDING SITE II or BINDING SITE III. Table 2 illustrates the complementarity of the nucleotide sequences of LOC90768 BINDING SITE, designated SEQ ID:2674, to the nucleotide sequence of VGAM148 RNA, herein designated VGAM RNA, also designated SEQ ID:483.

Another function of VGAM148 is therefore inhibition of LOC90768 (Accession XM_{—}033986). Accordingly, utilities of VGAM148 include diagnosis, prevention and treatment of diseases and clinical conditions associated with LOC90768. FIG. 1 further provides a conceptual description of a novel bioinformatically detected viral gene of the present invention, referred to here as Viral Genomic Address Messenger 149 (VGAM149) viral gene, which modulates expression of respective host target genes thereof, the function and utility of which host target genes is known in the art.

VGAM149 is a novel bioinformatically detected regulatory, non protein coding, viral micro RNA (miRNA) gene. The method by which VGAM149 was detected is described hereinabove with reference to FIGS. 1-8.

VGAM149 gene, herein designated VGAM GENE, is a viral gene contained in the genome of Vaccinia Virus. VGAM149 host target gene, herein designated VGAM HOST TARGET GENE, is a human gene contained in the human genome.

VGAM149 gene encodes a VGAM149 precursor RNA, herein designated VGAM PRECURSOR RNA. Similar to other miRNA genes, and unlike most ordinary genes, VGAM149 precursor RNA does not encode a protein. A nucleotide sequence identical or highly similar to the nucleotide sequence of VGAM149 precursor RNA is designated SEQ ID:135, and is provided hereinbelow with reference to the sequence listing part. Nucleotide sequence SEQ ID:135 is located at position 71497 relative to the genome of Vaccinia Virus.

VGAM149 precursor RNA folds onto itself, forming VGAM149 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM149 folded precursor RNA into VGAM149 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM149 RNA is designated SEQ ID:484, and is provided hereinbelow with reference to the sequence listing part.

VGAM149 host target gene, herein designated VGAM HOST TARGET GENE, encodes a corresponding messenger RNA, VGAM149 host target RNA, herein designated VGAM HOST TARGET RNA. VGAM149 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM149 RNA, herein designated VGAM RNA, binds complementarily to one or more host target binding sites located in untranslated regions of VGAM149 host target RNA, herein designated VGAM HOST TARGET RNA. This complementary binding is due to the fact that the nucleotide sequence of VGAM149 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM149 RNA, herein designated VGAM RNA, may have a different number of host target binding sites in untranslated regions of a VGAM149 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM149 RNA, herein designated VGAM RNA, to host target binding sites on VGAM149 host target RNA, herein designated VGAM HOST TARGET RNA, such as BINDING SITE I, BINDING SITE II and BINDING SITE III, inhibits translation of VGAM149 host target RNA into VGAM149 host target protein, herein designated VGAM HOST TARGET PROTEIN. VGAM host target protein is therefore outlined by a broken line.

It is appreciated that VGAM149 host target gene, herein designated VGAM HOST TARGET GENE, in fact represents a plurality of VGAM149 host target genes. The mRNA of each one of this plurality of VGAM149 host target genes comprises one or more host target binding sites, each having a nucleotide sequence which is at least partly complementary to VGAM149 RNA, herein designated VGAM RNA, and which when bound by VGAM149 RNA causes inhibition of translation of respective one or more VGAM149 host target proteins.

It is further appreciated by one skilled in the art that the mode of translational inhibition illustrated by FIG. 1 with specific reference to translational inhibition exerted by VGAM149 gene, herein designated VGAM GENE, on one or more VGAM149 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM149 is inhibition of expression of host target genes, as part of a novel viral mechanism of attacking a host. Accordingly, utilities of VGAM149 include diagnosis, prevention and treatment of viral infection by Vaccinia Virus. Specific functions, and accordingly utilities, of VGAM149 correlate with, and may be deduced from, the identity of the host target genes which VGAM149 binds and inhibits, and the function of these host target genes, as elaborated hereinbelow.

Nucleotide sequences of the VGAM149 precursor RNA, herein designated VGAM PRECURSOR RNA, and of the ‘diced’ VGAM149 RNA, herein designated VGAM RNA, and a schematic representation of the secondary folding of VGAM149 folded precursor RNA, herein designated VGAM FOLDED PRECURSOR RNA, of VGAM149 are further described hereinbelow with reference to Table 1.

Nucleotide sequences of host target binding sites, such as BINDING SITE-I, BINDING SITE-II and BINDING SITE-III of FIG. 1, found on VGAM149 host target RNA, and schematic representation of the complementarity of each of these host target binding sites to VGAM149 RNA, herein designated VGAM RNA, are described hereinbelow with reference to Table 2.

As mentioned hereinabove with reference to FIG. 1, a function of VGAM149 gene, herein designated VGAM is inhibition of expression of VGAM149 target genes. It is appreciated that specific functions, and accordingly utilities, of VGAM149 correlate with, and may be deduced from, the identity of the target genes which VGAM149 binds and inhibits, and the function of these target genes, as elaborated hereinbelow.

Alanyl (membrane) Aminopeptidase (aminopeptidase N, aminopeptidase M, microsomal aminopeptidase, CD13, p150) (ANPEP, Accession NM_{—}001150) is a VGAM149 host target gene. ANPEP BINDING SITE is HOST TARGET binding site found in the 3′ untranslated region of mRNA encoded by ANPEP, corresponding to a HOST TARGET binding site such as BINDING SITE I, BINDING SITE II or BINDING SITE III. Table 2 illustrates the complementarity of the nucleotide sequences of ANPEP BINDING SITE, designated SEQ ID:805, to the nucleotide sequence of VGAM149 RNA, herein designated VGAM RNA, also designated SEQ ID:484.

A function of VGAM149 is therefore inhibition of Alanyl (membrane) Aminopeptidase (aminopeptidase N, aminopeptidase M, microsomal aminopeptidase, CD13, p150) (ANPEP, Accession NM_{—}001150), a gene which is a cell-surface transmembrane glycosylated metalloprotease that processes signaling peptides. Accordingly, utilities of VGAM149 include diagnosis, prevention and treatment of diseases and clinical conditions associated with ANPEP. The function of ANPEP has been established by previous studies. A surface antigen glycoprotein of molecular weight about 150,000 is recognized by monoclonal antibodies MY7 and MCS2. Look et al. (1986) isolated the complete ANPEP gene, which they called GP150, from a human placental genomic library. By study of somatic cell hybrid DNA and by in situ hybridization, the GP150 gene was assigned to 15q25-q26. This chromosomal location coincides with that of the oncogene FES (OMIM Ref. No. 190030), which is also expressed in myeloid cells. They are separate loci, however, because FES probes do not hybridize to cloned sequences spanning the GP150 gene and the 2 genes have different restriction maps. Like FES, GP150 is distal to the breakpoint in t(15;17)(q22;q21.1) of acute promyelocytic leukemia. By analysis of mouse-human somatic cell hybrids, Watt and Willard (1990) assigned the ANPEP gene, which they called PEPN, to 15q11-qter. With a genomic DNA probe, they detected a frequent DraIII polymorphism useful as a marker for human chromosome 15. Look et al. (1989) determined the complete primary structure of GP150, known as CD13. The large extracellular carboxyterminal domain contained a pentapeptide consensus sequence characteristic of members of the zinc-binding metalloproteinase super family. Sequence comparisons with known enzymes of this class showed that CD13 and aminopeptidase N are identical. The latter enzyme was thought to be involved in the metabolism of regulatory peptides by diverse cell types, including small intestinal and renal tubular epithelial cells, macrophages, granulocytes, and synaptic membranes from the CNS.

Full details of the abovementioned studies are described in the following publications, the disclosure of which are hereby incorporated by reference:
Look, A. T.; Ashmun, R. A.; Shapiro, L. H.; Peiper, S. C.: Human myeloid plasma membrane glycoprotein CD13 (gp150) is identical to aminopeptidase N. J. Clin. Invest. 83: 1299-1307, 1989.; and
Look, A. T.; Peiper, S. C.; Rebentisch, M. B.; Ashmun, R. A.; Roussel, M. F.; Lemons, R. S.; Le Beau, M. M.; Rubin, C. M.; Sherr, C. J.: Molecular cloning, expression, and chromosomal.

Further studies establishing the function and utilities of ANPEP are found in John Hopkins OMIM database record ID 151530, and in sited publications numbered 1156-1161 listed in the bibliography section hereinbelow, which are also hereby incorporated by reference. Nucleoside Phosphorylase (NP, Accession NM_{—}000270) is another VGAM149 host target gene. NP BINDING SITE is HOST TARGET binding site found in the 3′ untranslated region of mRNA encoded by NP, corresponding to a HOST TARGET binding site such as BINDING SITE I, BINDING SITE II or BINDING SITE III. Table 2 illustrates the complementarity of the nucleotide sequences of NP BINDING SITE, designated SEQ ID:724, to the nucleotide sequence of VGAM149 RNA, herein designated VGAM RNA, also designated SEQ ID:484.

Another function of VGAM149 is therefore inhibition of Nucleoside Phosphorylase (NP, Accession NM_{—}000270). Accordingly, utilities of VGAM149 include diagnosis, prevention and treatment of diseases and clinical conditions associated with NP. Transcription Factor AP-2 Gamma (activating enhancer binding protein 2 gamma) (TFAP2C, Accession NM_{—}003222) is another VGAM149 host target gene. TFAP2C BINDING SITE is HOST TARGET binding site found in the 3′ untranslated region of mRNA encoded by TFAP2C, corresponding to a HOST TARGET binding site such as BINDING SITE I, BINDING SITE II or BINDING SITE III. Table 2 illustrates the complementarity of the nucleotide sequences of TFAP2C BINDING SITE, designated SEQ ID: 1000, to the nucleotide sequence of VGAM149 RNA, herein designated VGAM RNA, also designated SEQ ID:484.

Another function of VGAM149 is therefore inhibition of Transcription Factor AP-2 Gamma (activating enhancer binding protein 2 gamma) (TFAP2C, Accession NM_{—}003222), a gene which is a sequence-specific dna-binding protein that interacts with inducible viral and cellular enhancer elements to regulate transcription of selected genes. Accordingly, utilities of VGAM149 include diagnosis, prevention and treatment of diseases and clinical conditions associated with TFAP2C. The function of TFAP2C has been established by previous studies. Families of related transcription factors are often expressed in the same cell lineages but at different times or sites in the developing embryo. The AP-2 family appears to regulate the expression of genes required for development of tissues of ectodermal origin such as neural crest and skin (Williamson et al., 1996). AP-2 may also be involved in the overexpression of c-erbB-2 (OMIM Ref. No. 164870) in human breast cancer cells (Bosher et al., 1995). Williamson et al. (1996) isolated an AP-2-related cDNA. The predicted protein differs from AP-2-alpha (OMIM Ref. No. 107580) and -beta (OMIM Ref. No. 601601) in the N-terminal activation domain, but is 75 to 85% conserved within the DNA-binding and dimerization domains. All 3 gene products (AP-2-alpha, -beta, and -gamma) bind the GCCNNNGGC motif. Williamson et al. (1996) also obtained a genomic clone for AP-2-gamma (designated TFAP2C). They showed it to have a similar gene structure to TFAP2A and mapped it by fluorescence in situ hybridization to 20q13.2. A mouse genomic clone was used to map the mouse Tcfap2c locus to 2H3-4.

Full details of the abovementioned studies are described in the following publications, the disclosure of which are hereby incorporated by reference:
Bosher, J. M.; Williams, T.; Hurst, H. C.: The developmentally regulated transcription factor AP-2 is involved in cerbB-2 overexpression in human mammary carcinoma. Proc. Nat. Acad. Sci. 92: 744-747, 1995.; and
Williamson, J. A.; Bosher, J. M.; Skinner, A.; Sheer, D.; Williams, T.; Hurst, H. C.: Chromosomal mapping of the human and mouse homologues of two new members of the AP-2 family of tra.

Further studies establishing the function and utilities of TFAP2C are found in John Hopkins OMIM database record ID 601602, and in sited publications numbered 2094 listed in the bibliography section hereinbelow, which are also hereby incorporated by reference. Zinc Finger Protein 83 (HPF1) (ZNF83, Accession NM_{—}018300) is another VGAM149 host target gene. ZNF83 BINDING SITE is HOST TARGET binding site found in the 5′ untranslated region of mRNA encoded by ZNF83, corresponding to a HOST TARGET binding site such as BINDING SITE I, BINDING SITE II or BINDING SITE III. Table 2 illustrates the complementarity of the nucleotide sequences of ZNF83 BINDING SITE, designated SEQ ID:1813, to the nucleotide sequence of VGAM149 RNA, herein designated VGAM RNA, also designated SEQ ID:484.

Another function of VGAM149 is therefore inhibition of Zinc Finger Protein 83 (HPF1) (ZNF83, Accession NM_{—}018300). Accordingly, utilities of VGAM149 include diagnosis, prevention and treatment of diseases and clinical conditions associated with ZNF83. MGC26766 (Accession NM_{—}145021) is another VGAM149 host target gene. MGC26766 BINDING SITE is HOST TARGET binding site found in the 3′ untranslated region of mRNA encoded by MGC26766, corresponding to a HOST TARGET binding site such as BINDING SITE I, BINDING SITE II or BINDING SITE III. Table 2 illustrates the complementarity of the nucleotide sequences of MGC26766 BINDING SITE, designated SEQ ID:2509, to the nucleotide sequence of VGAM149 RNA, herein designated VGAM RNA, also designated SEQ ID:484.

Another function of VGAM149 is therefore inhibition of MGC26766 (Accession NM_{—}145021). Accordingly, utilities of VGAM149 include diagnosis, prevention and treatment of diseases and clinical conditions associated with MGC26766. SCYB10 (Accession NM_{—}001565) is another VGAM149 host target gene. SCYB10 BINDING SITE is HOST TARGET binding site found in the 3′ untranslated region of mRNA encoded by SCYB10, corresponding to a HOST TARGET binding site such as BINDING SITE I, BINDING SITE II or BINDING SITE III. Table 2 illustrates the complementarity of the nucleotide sequences of SCYB10 BINDING SITE, designated SEQ ID:836, to the nucleotide sequence of VGAM149 RNA, herein designated VGAM RNA, also designated SEQ ID:484.

Another function of VGAM149 is therefore inhibition of SCYB10 (Accession NM_{—}001565). Accordingly, utilities of VGAM149 include diagnosis, prevention and treatment of diseases and clinical conditions associated with SCYB10. FIG. 1 further provides a conceptual description of a novel bioinformatically detected viral gene of the present invention, referred to here as Viral Genomic Address Messenger 150 (VGAM150) viral gene, which modulates expression of respective host target genes thereof, the function and utility of which host target genes is known in the art.

VGAM150 is a novel bioinformatically detected regulatory, non protein coding, viral micro RNA (miRNA) gene. The method by which VGAM150 was detected is described hereinabove with reference to FIGS. 1-8.

VGAM150 gene, herein designated VGAM GENE, is a viral gene contained in the genome of Vaccinia Virus. VGAM150 host target gene, herein designated VGAM HOST TARGET GENE, is a human gene contained in the human genome.

VGAM150 gene encodes a VGAM150 precursor RNA, herein designated VGAM PRECURSOR RNA. Similar to other miRNA genes, and unlike most ordinary genes, VGAM150 precursor RNA does not encode a protein. A nucleotide sequence identical or highly similar to the nucleotide sequence of VGAM150 precursor RNA is designated SEQ ID:136, and is provided hereinbelow with reference to the sequence listing part. Nucleotide sequence SEQ ID:136 is located at position 73985 relative to the genome of Vaccinia Virus.

VGAM150 precursor RNA folds onto itself, forming VGAM150 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM150 folded precursor RNA into VGAM150 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 93%) nucleotide sequence of VGAM150 RNA is designated SEQ ID:485, and is provided hereinbelow with reference to the sequence listing part.

VGAM150 host target gene, herein designated VGAM HOST TARGET GENE, encodes a corresponding messenger RNA, VGAM150 host target RNA, herein designated VGAM HOST TARGET RNA. VGAM150 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM150 RNA, herein designated VGAM RNA, binds complementarily to one or more host target binding sites located in untranslated regions of VGAM150 host target RNA, herein designated VGAM HOST TARGET RNA. This complementary binding is due to the fact that the nucleotide sequence of VGAM150 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM150 RNA, herein designated VGAM RNA, may have a different number of host target binding sites in untranslated regions of a VGAM150 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM150 RNA, herein designated VGAM RNA, to host target binding sites on VGAM150 host target RNA, herein designated VGAM HOST TARGET RNA, such as BINDING SITE I, BINDING SITE II and BINDING SITE III, inhibits translation of VGAM150 host target RNA into VGAM150 host target protein, herein designated VGAM HOST TARGET PROTEIN. VGAM host target protein is therefore outlined by a broken line.

It is appreciated that VGAM150 host target gene, herein designated VGAM HOST TARGET GENE, in fact represents a plurality of VGAM150 host target genes. The mRNA of each one of this plurality of VGAM150 host target genes comprises one or more host target binding sites, each having a nucleotide sequence which is at least partly complementary to VGAM150 RNA, herein designated VGAM RNA, and which when bound by VGAM150 RNA causes inhibition of translation of respective one or more VGAM150 host target proteins.

It is further appreciated by one skilled in the art that the mode of translational inhibition illustrated by FIG. 1 with specific reference to translational inhibition exerted by VGAM150 gene, herein designated VGAM GENE, on one or more VGAM150 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM150 is inhibition of expression of host target genes, as part of a novel viral mechanism of attacking a host. Accordingly, utilities of VGAM150 include diagnosis, prevention and treatment of viral infection by Vaccinia Virus. Specific functions, and accordingly utilities, of VGAM150 correlate with, and may be deduced from, the identity of the host target genes which VGAM150 binds and inhibits, and the function of these host target genes, as elaborated hereinbelow.

Nucleotide sequences of the VGAM150 precursor RNA, herein designated VGAM PRECURSOR RNA, and of the ‘diced’ VGAM150 RNA, herein designated VGAM RNA, and a schematic representation of the secondary folding of VGAM150 folded precursor RNA, herein designated VGAM FOLDED PRECURSOR RNA, of VGAM150 are further described hereinbelow with reference to Table 1.

Nucleotide sequences of host target binding sites, such as BINDING SITE-I, BINDING SITE-II and BINDING SITE-III of FIG. 1, found on VGAM150 host target RNA, and schematic representation of the complementarity of each of these host target binding sites to VGAM150 RNA, herein designated VGAM RNA, are described hereinbelow with reference to Table 2.

As mentioned hereinabove with reference to FIG. 1, a function of VGAM150 gene, herein designated VGAM is inhibition of expression of VGAM150 target genes. It is appreciated that specific functions, and accordingly utilities, of VGAM150 correlate with, and may be deduced from, the identity of the target genes which VGAM150 binds and inhibits, and the function of these target genes, as elaborated hereinbelow.

Endometrial Bleeding Associated Factor (left-right determination, factor A; transforming growth factor beta super family) (EBAF, Accession XM_{—}037302) is a VGAM150 host target gene. EBAF BINDING SITE is HOST TARGET binding site found in the 3′ untranslated region of mRNA encoded by EBAF, corresponding to a HOST TARGET binding site such as BINDING SITE I, BINDING SITE II or BINDING SITE III. Table 2 illustrates the complementarity of the nucleotide sequences of EBAF BINDING SITE, designated SEQ ID:2721, to the nucleotide sequence of VGAM150 RNA, herein designated VGAM RNA, also designated SEQ ID:485.

A function of VGAM150 is therefore inhibition of Endometrial Bleeding Associated Factor (left-right determination, factor A; transforming growth factor beta super family) (EBAF, Accession XM_{—}037302), a gene which LEFT-RIGHT AXIS MALFORMATIONS. Accordingly, utilities of VGAM150 include diagnosis, prevention and treatment of diseases and clinical conditions associated with EBAF. The function of EBAF has been established by previous studies. Because of the possibility that Lefty mutations may be associated with human L-R axis malformations, Kosaki et al. (1999) characterized 2 human homologs, LEFTY A and LEFTY B (OMIM Ref. No. 603037). PCR screening of a PAC genomic library identified a clone that contained both LEFTY A and LEFTY B genes. Restriction mapping showed that the genes are separated by approximately 50 kb and are oriented in tandem. The 2 genes were localized by FISH to 1q42, a region syntenic to the location to which the mouse Lefty genes have been mapped at 1H5 (Meno et al., 1997). Both LEFTY A and LEFTY B contain 4 exons which are spliced at identical positions, and both genes encode proteins with 366 amino acids. LEFTY A was found to be identical to EBAF, the cDNA previously identified by Kothapalli et al. (1997). The deduced amino acid sequences of LEFTY A and LEFTY B are more similar to each other than to Lefty-1 or Lefty-2 of the mouse. Analysis of 126 human cases of L-R axis malformation showed 1 nonsense and 1 missense mutation in the LEFTY A gene. Both mutations lay in the cysteine-knot region of the LEFTY A protein, and the phenotype of affected individuals was very similar to that typically seen in Lefty-1 −/− mice with L-R axis malformations. Because of the possibility that Lefty mutations may be associated with human L-R axis malformations, Kosaki et al. (1999) characterized 2 human homologs, LEFTY A and LEFTY B (OMIM Ref. No. 603037). PCR screening of a PAC genomic library identified a clone that contained both LEFTY A and LEFTY B genes. Restriction mapping showed that the genes are separated by approximately 50 kb and are oriented in tandem. The 2 genes were localized by FISH to 1q42, a region syntenic to the location to which the mouse Lefty genes have been mapped at 1H5 (Meno et al., 1997). Both LEFTY A and LEFTY B contain 4 exons which are spliced at identical positions, and both genes encode proteins with 366 amino acids. LEFTY A was found to be identical to EBAF, the cDNA previously identified by Kothapalli et al. (1997). The deduced amino acid sequences of LEFTY A and LEFTY B are more similar to each other than to Lefty-1 or Lefty-2 of the mouse. Analysis of 126 human cases of L-R axis malformation showed 1 nonsense and 1 missense mutation in the LEFTY A gene. Both mutations lay in the cysteine-knot region of the LEFTY A protein, and the phenotype of affected individuals was very similar to that typically seen in Lefty-1 −/− mice with L-R axis malformations. Animal model experiments lend further support to the function of EBAF. Lefty-1, lefty-2, and nodal (OMIM Ref. No. 601265) are expressed on the left side of developing mouse embryos and are implicated in L-R determination. Meno et al. (1998) examined the role of lefty-1 by analyzing mutant mice lacking this gene. The lefty-1-deficient mice showed a variety of L-R positional defects in visceral organs. The most common feature of lefty-1 −/− mice was thoracic left isomerism (rather than right isomerism). The lack of lefty-1 resulted in bilateral expression of nodal, lefty-2, and Pitx2 (OMIM Ref. No. 601542), a homeo box gene normally expressed on the left side. These observations suggested that the role of lefty-1 is to restrict the expression of lefty-2 and nodal to the left side, and that lefty-2 or nodal encode a signal for ‘leftness.’

It is appreciated that the abovementioned animal model for EBAF is acknowledged by those skilled in the art as a scientifically valid animal model, as can be further appreciated from the publications sited hereinbelow.

Full details of the abovementioned studies are described in the following publications, the disclosure of which are hereby incorporated by reference:
Kosaki, K.; Bassi, M. T.; Kosaki, R.; Lewin, M.; Belmont, J.; Schauer, G.; Casey, B.: Characterization and mutation analysis of human LEFTY A and LEFTY B, homologues of murine genes implicated in left-right axis development. Am. J. Hum. Genet. 64: 712-721, 1999.; and
Meno, C.; Shimono, A.; Saijoh, Y.; Yashiro, K.; Mochida, K.; Ohishi, S.; Noji, S.; Kondoh, H.; Hamada, H.: Lefty-1 is required for left-right determination as a regulator of lefty-2 an.

Further studies establishing the function and utilities of EBAF are found in John Hopkins OMIM database record ID 601877, and in sited publications numbered 2738-325 listed in the bibliography section hereinbelow, which are also hereby incorporated by reference. Coagulation Factor VII (serum prothrombin conversion accelerator) (F7, Accession NM_{—}000131) is another VGAM150 host target gene. F7 BINDING SITE1 and F7 BINDING SITE2 are HOST TARGET binding sites found in untranslated regions of mRNA encoded by F7, corresponding to HOST TARGET binding sites such as BINDING SITE I, BINDING SITE II or BINDING SITE III. Table 2 illustrates the complementarity of the nucleotide sequences of F7 BINDING SITE1 and F7 BINDING SITE2, designated SEQ ID:706 and SEQ ID:1885 respectively, to the nucleotide sequence of VGAM150 RNA, herein designated VGAM RNA, also designated SEQ ID:485.

Another function of VGAM150 is therefore inhibition of Coagulation Factor VII (serum prothrombin conversion accelerator) (F7, Accession NM_{—}000131). Accordingly, utilities of VGAM150 include diagnosis, prevention and treatment of diseases and clinical conditions associated with F7. LOC146438 (Accession XM_{—}085464) is another VGAM150 host target gene. LOC146438 BINDING SITE is HOST TARGET binding site found in the 3′ untranslated region of mRNA encoded by LOC146438, corresponding to a HOST TARGET binding site such as BINDING SITE I, BINDING SITE II or BINDING SITE III. Table 2 illustrates the complementarity of the nucleotide sequences of LOC146438 BINDING SITE, designated SEQ ID:3091, to the nucleotide sequence of VGAM150 RNA, herein designated VGAM RNA, also designated SEQ ID:485.

Another function of VGAM150 is therefore inhibition of LOC146438 (Accession XM_{—}085464). Accordingly, utilities of VGAM150 include diagnosis, prevention and treatment of diseases and clinical conditions associated with LOC146438. FIG. 1 further provides a conceptual description of a novel bioinformatically detected viral gene of the present invention, referred to here as Viral Genomic Address Messenger 151 (VGAM151) viral gene, which modulates expression of respective host target genes thereof, the function and utility of which host target genes is known in the art.

VGAM151 is a novel bioinformatically detected regulatory, non protein coding, viral micro RNA (miRNA) gene. The method by which VGAM151 was detected is described hereinabove with reference to FIGS. 1-8.

VGAM151 gene, herein designated VGAM GENE, is a viral gene contained in the genome of Vaccinia Virus. VGAM151 host target gene, herein designated VGAM HOST TARGET GENE, is a human gene contained in the human genome.

VGAM151 gene encodes a VGAM151 precursor RNA, herein designated VGAM PRECURSOR RNA. Similar to other miRNA genes, and unlike most ordinary genes, VGAM151 precursor RNA does not encode a protein. A nucleotide sequence identical or highly similar to the nucleotide sequence of VGAM151 precursor RNA is designated SEQ ID:137, and is provided hereinbelow with reference to the sequence listing part. Nucleotide sequence SEQ ID:137 is located at position 72599 relative to the genome of Vaccinia Virus.

VGAM151 precursor RNA folds onto itself, forming VGAM151 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM151 folded precursor RNA into VGAM151 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM151 RNA is designated SEQ ID:486, and is provided hereinbelow with reference to the sequence listing part.

VGAM151 host target gene, herein designated VGAM HOST TARGET GENE, encodes a corresponding messenger RNA, VGAM151 host target RNA, herein designated VGAM HOST TARGET RNA. VGAM151 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM151 RNA, herein designated VGAM RNA, binds complementarily to one or more host target binding sites located in untranslated regions of VGAM151 host target RNA, herein designated VGAM HOST TARGET RNA. This complementary binding is due to the fact that the nucleotide sequence of VGAM151 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM151 RNA, herein designated VGAM RNA, may have a different number of host target binding sites in untranslated regions of a VGAM151 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM151 RNA, herein designated VGAM RNA, to host target binding sites on VGAM151 host target RNA, herein designated VGAM HOST TARGET RNA, such as BINDING SITE I, BINDING SITE II and BINDING SITE III, inhibits translation of VGAM151 host target RNA into VGAM151 host target protein, herein designated VGAM HOST TARGET PROTEIN. VGAM host target protein is therefore outlined by a broken line.

It is appreciated that VGAM151 host target gene, herein designated VGAM HOST TARGET GENE, in fact represents a plurality of VGAM151 host target genes. The mRNA of each one of this plurality of VGAM151 host target genes comprises one or more host target binding sites, each having a nucleotide sequence which is at least partly complementary to VGAM151 RNA, herein designated VGAM RNA, and which when bound by VGAM151 RNA causes inhibition of translation of respective one or more VGAM151 host target proteins.

It is further appreciated by one skilled in the art that the mode of translational inhibition illustrated by FIG. 1 with specific reference to translational inhibition exerted by VGAM151 gene, herein designated VGAM GENE, on one or more VGAM151 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM151 is inhibition of expression of host target genes, as part of a novel viral mechanism of attacking a host. Accordingly, utilities of VGAM151 include diagnosis, prevention and treatment of viral infection by Vaccinia Virus. Specific functions, and accordingly utilities, of VGAM151 correlate with, and may be deduced from, the identity of the host target genes which VGAM151 binds and inhibits, and the function of these host target genes, as elaborated hereinbelow.

Nucleotide sequences of the VGAM151 precursor RNA, herein designated VGAM PRECURSOR RNA, and of the ‘diced’ VGAM151 RNA, herein designated VGAM RNA, and a schematic representation of the secondary folding of VGAM151 folded precursor RNA, herein designated VGAM FOLDED PRECURSOR RNA, of VGAM151 are further described hereinbelow with reference to Table 1.

Nucleotide sequences of host target binding sites, such as BINDING SITE-I, BINDING SITE-II and BINDING SITE-III of FIG. 1, found on VGAM151 host target RNA, and schematic representation of the complementarity of each of these host target binding sites to VGAM151 RNA, herein designated VGAM RNA, are described hereinbelow with reference to Table 2.

As mentioned hereinabove with reference to FIG. 1, a function of VGAM151 gene, herein designated VGAM is inhibition of expression of VGAM151 target genes. It is appreciated that specific functions, and accordingly utilities, of VGAM151 correlate with, and may be deduced from, the identity of the target genes which VGAM151 binds and inhibits, and the function of these target genes, as elaborated hereinbelow.

2,3-bisphosphoglycerate Mutase (BPGM, Accession NM_{—}001724) is a VGAM151 host target gene. BPGM BINDING SITE is HOST TARGET binding site found in the 3′ untranslated region of mRNA encoded by BPGM, corresponding to a HOST TARGET binding site such as BINDING SITE I, BINDING SITE II or BINDING SITE III. Table 2 illustrates the complementarity of the nucleotide sequences of BPGM BINDING SITE, designated SEQ ID:852, to the nucleotide sequence of VGAM151 RNA, herein designated VGAM RNA, also designated SEQ ID:486.

A function of VGAM151 is therefore inhibition of 2,3-bisphosphoglycerate Mutase (BPGM, Accession NM_{—}001724), a gene which plays a role in regulating hemoglobin oxygen affinity. Accordingly, utilities of VGAM151 include diagnosis, prevention and treatment of diseases and clinical conditions associated with BPGM. The function of BPGM has been established by previous studies. Chen et al. (1971) described a genetically determined electrophoretic variant of 2,3-diphosphoglycerate mutase in a Canadian Eskimo family. The findings in heterozygotes were consistent with the view that the protein is a dimer of 2 identical subunits. Scott and Wright (1982) found DPGM to be polymorphic in 4 Alaskan ethnic groups. Hemoglobin and hematocrit were elevated in all deficient persons. Thus, both hemolytic anemia and polycythemia have been observed with deficiency of DPGM. Rosa et al. (1973, 1978) showed that the DPGM and 2,3-bisphosphoglycerate phosphatase activities of red cells are due to a single enzyme, bisphosphoglycerate mutase (EC 5.4.2.4). Joulin et al. (1986) cloned and sequenced cDNA for human red cell 2,3-bisphosphoglycerate mutase (EC 2.7.5.4). They presented a revised amino acid sequence of human BPGM based on the nucleotide sequence data. BPGM shows some phosphoglycerate mutase activity (Sasaki et al., 1975); nevertheless, the major portion of PGAM activity in the red cells is expressed by PGAMA (OMIM Ref. No. 172250), a protein genetically distinct from BPGM but structurally related to it. The PGAMA locus is situated on chromosome 10. Using a cDNA clone for human BPGM in in situ hybridization experiments, Joulin et al. (1987) and Barichard et al. (1987) mapped the BPGM gene to 7q22-q34. Joulin et al. (1988) isolated the 2,3-bisphosphoglycerate mutase gene from genomic libraries. By Southern blots and DNA sequencing, they determined that it extends over 22 kb and is composed of 2 introns and 3 exons. The second exon correlates with a functional subdomain of the protein. No GC-rich sequence or GC box was found in the 5-prime flanking region of the gene. Both amino acid and cDNA sequence studies show that DPGAM is homologous to PGAM (172250, 261670) (Joulin et al., 1986; Yanagawa et al., 1986).

Full details of the abovementioned studies are described in the following publications, the disclosure of which are hereby incorporated by reference:
Rosa, R.; Prehu, M.-O.; Beuzard, Y.; Rosa, J.: The first case of a complete deficiency of diphosphoglycerate mutase in human erythrocytes. J. Clin. Invest. 62: 907-915, 1978.; and
Joulin, V.; Peduzzi, J.; Romeo, P.-H.; Rosa, R.; Valentin, C.; Dubart, A.; Lapeyre, B.; Blouquit, Y.; Garel, M.-C.; Goossens, M.; Rosa, J.; Cohen-Solal, M.: Molecular cloning and sequence.

Further studies establishing the function and utilities of BPGM are found in John Hopkins OMIM database record ID 222800, and in sited publications numbered 2250-2252, 401, 2253-2258, 14, 2259-2261, 88 and 2262-2265 listed in the bibliography section hereinbelow, which are also hereby incorporated by reference. Hyaluronan Synthase 2 (HAS2, Accession NM_{—}005328) is another VGAM151 host target gene. HAS2 BINDING SITE is HOST TARGET binding site found in the 3′ untranslated region of mRNA encoded by HAS2, corresponding to a HOST TARGET binding site such as BINDING SITE I, BINDING SITE II or BINDING SITE III. Table 2 illustrates the complementarity of the nucleotide sequences of HAS2 BINDING SITE, designated SEQ ID:1198, to the nucleotide sequence of VGAM151 RNA, herein designated VGAM RNA, also designated SEQ ID:486.

Another function of VGAM151 is therefore inhibition of Hyaluronan Synthase 2 (HAS2, Accession NM_{—}005328), a gene which plays a role in hyaluronan/hyaluronic acid (ha) synthesis and transport. Accordingly, utilities of VGAM151 include diagnosis, prevention and treatment of diseases and clinical conditions associated with HAS2. The function of HAS2 has been established by previous studies. Hyaluronan, or hyaluronic acid (HA), is a high molecular weight unbranched polysaccharide of the extracellular matrix. Watanabe and Yamaguchi (1996) described the cloning of a human cDNA from a fibroblast library using degenerate PCR with primers based on regions of conservation between the previously published Xenopus DG42 and Streptococcus HasA proteins. When expressed in cell culture, the cDNA increased hyaluronan production. The sequence of the predicted 552-amino acid protein differs from HAS1 (OMIM Ref. No. 601463) and so was designated HAS2. The HAS2 amino acid sequence is 55% similar to the Xenopus DG42 sequence and 55% identical to mouse Has1. Northern blots showed high levels of HAS2 mRNA in a proliferating human fibroblast cell line but not in growth-arrested cells. Watanabe and Yamaguchi (1996) speculated that if HAS2 is not a true hyaluronan synthase, it is at least a major inducer of HA synthase activity. Spicer et al. (1996) isolated the apparent mouse homolog of human HAS2 from a mouse embryo cDNA library using degenerate PCR. The predicted mouse protein is also 552 amino acids long. The Has2 protein is predicted to contain multiple transmembrane domains similar to bacterial HasA and mammalian HAS1. Northern blots demonstrated 4.8- and 3.2-kb transcripts expressed highly in the mouse embryo and at lower levels in adult heart, brain, spleen, lung, and skeletal muscle. When expressed in COS cells, the cDNA was shown to induce the formation of large HA coats around the cells. Based on analogy with what is known about HA production in Streptococcus, Spicer et al. (1996) suggested that HAS2 may play a key role in HA transport rather than act as a synthase per se.

Full details of the abovementioned studies are described in the following publications, the disclosure of which are hereby incorporated by reference:
Watanabe, K.; Yamaguchi, Y.: Molecular identification of a putative human hyaluronan synthase. J. Biol. Chem. 271: 22945-22948, 1996.; and
Spicer, A. P.; Augustine, M. L.; McDonald, J. A.: Molecular cloning and characterization of a putative mouse hyaluronan synthase. J. Biol. Chem. 271: 23400-23406, 1996.

Further studies establishing the function and utilities of HAS2 are found in John Hopkins OMIM database record ID 601636, and in sited publications numbered 1551-630 listed in the bibliography section hereinbelow, which are also hereby incorporated by reference. Histone Deacetylase 2 (HDAC2, Accession XM_{—}165684) is another VGAM151 host target gene. HDAC2 BINDING SITE is HOST TARGET binding site found in the 3′ untranslated region of mRNA encoded by HDAC2, corresponding to a HOST TARGET binding site such as BINDING SITE I, BINDING SITE II or BINDING SITE III. Table 2 illustrates the complementarity of the nucleotide sequences of HDAC2 BINDING SITE, designated SEQ ID:3507, to the nucleotide sequence of VGAM151 RNA, herein designated VGAM RNA, also designated SEQ ID:486.

Another function of VGAM151 is therefore inhibition of Histone Deacetylase 2 (HDAC2, Accession XM_{—}165684), a gene which is responsible for the deacetylation of lysine residues on the n-terminal part of the core histones and mediates transcriptional repression. Accordingly, utilities of VGAM151 include diagnosis, prevention and treatment of diseases and clinical conditions associated with HDAC2. The function of HDAC2 has been established by previous studies. Zhang et al. (1998) identified the SIN3-associated 30-kD protein SAP30 (OMIM Ref. No. 603378) as a novel component of the human histone deacetylase complex which is conserved among eukaryotic organisms. This complex includes SIN3, SAP18 (OMIM Ref. No. 602949), the histone deacetylases HDAC1 (OMIM Ref. No. 601241) and HDAC2, the histone-binding proteins RbAp46 (RBBP7; 602922) and RbAp48 (RBBP4; 602923), as well as other polypeptides. Yarden and Brody (1999) reported that BRCA1 (OMIM Ref. No. 113705) interacts in vivo and in vitro with RBBP7 and RBBP4, with RB1 (OMIM Ref. No. 180200), and that the BRCT domain of BRCA1 associates with HDAC1 and HDAC2. Rountree et al. (2000) showed that DNMT1 (OMIM Ref. No. 126375) can establish a repressive transcription complex consisting of DNMT1, HDAC2, and DMAP1 (OMIM Ref. No. 605077). The noncatalytic amino terminus of DNMT1 binds to HDAC2 and to DMAP1 and can mediate transcriptional repression. DMAP1 is targeted to replication foci through interaction with the far N terminus of DNMT1 throughout S phase, whereas HDAC2 joins DNMT1 and DMAP1 only during late S phase, providing a platform for how histones may become deacetylated in heterochromatin following replication.

Full details of the abovementioned studies are described in the following publications, the disclosure of which are hereby incorporated by reference:
Yarden, R. I.; Brody, L. C.: BRCA1 interacts with components of the histone deacetylase complex. Proc. Nat. Acad. Sci. 96: 4983-4988, 1999.; and
Rountree, M. R.; Bachman, K. E.; Baylin, S. B.: DNMT1 binds HDAC2 and a new co-repressor, DMAP1, to form a complex at replication foci. Nature Genet. 25: 269-277, 2000.

Further studies establishing the function and utilities of HDAC2 are found in John Hopkins OMIM database record ID 605164, and in sited publications numbered 971-973, 793, 974-97 and 1697 listed in the bibliography section hereinbelow, which are also hereby incorporated by reference. X-ray Repair Complementing Defective Repair In Chinese Hamster Cells 2 (XRCC2, Accession NM_{—}005431) is another VGAM151 host target gene. XRCC2 BINDING SITE is HOST TARGET binding site found in the 3′ untranslated region of mRNA encoded by XRCC2, corresponding to a HOST TARGET binding site such as BINDING SITE I, BINDING SITE II or BINDING SITE III. Table 2 illustrates the complementarity of the nucleotide sequences of XRCC2 BINDING SITE, designated SEQ ID:1208, to the nucleotide sequence of VGAM151 RNA, herein designated VGAM RNA, also designated SEQ ID:486.

Another function of VGAM151 is therefore inhibition of X-ray Repair Complementing Defective Repair In Chinese Hamster Cells 2 (XRCC2, Accession NM_{—}005431), a gene which involves in the homologous recombination repair (hrr) pathway of double-stranded dna. Accordingly, utilities of VGAM151 include diagnosis, prevention and treatment of diseases and clinical conditions associated with XRCC2. The function of XRCC2 has been established by previous studies. Johnson et al. (1999) demonstrated that XRCC2 is essential for the efficient repair of DNA double-strand breaks by homologous recombination between sister chromatids. Hamster cells deficient in XRCC2 showed a more than 100-fold decrease in homologous recombination induced by double-strand breaks compared with the parental cell line. This defect was corrected to almost wildtype levels by transient transfection with a plasmid expressing XRCC2. The repair defect in XRCC2 mutant cells appeared to be restricted to recombinational repair because nonhomologous end joining was normal. Johnson et al. (1999) concluded that XRCC2 is involved in the repair of DNA double-strand breaks by homologous recombination. Using a yeast 2-hybrid assay, Braybrooke et al. (2000) identified a direct interaction between XRCC2 and RAD51L3 (OMIM Ref. No. 602954), and they confirmed the interaction by pull-down assays between recombinant XRCC2 and endogenous RAD51L3 in HeLa cell extracts. Size-exclusion chromatography followed by Western blot analysis suggested that the 2 proteins exist as a heterodimer of about 70 kD.

Full details of the abovementioned studies are described in the following publications, the disclosure of which are hereby incorporated by reference:
Johnson, R. D.; Liu, N.; Jasin, M.: Mammalian XRCC2 promotes the repair of DNA double-strand breaks by homologous recombination. Nature 401: 397-399, 1999.; and
Braybrooke, J. P.; Spink, K. G.; Thacker, J.; Hickson, I. D.: The RAD51 family member, RAD51L3, is a DNA-stimulated ATPase that forms a complex with XRCC2. J. Biol. Chem. 275: 29100-29.

Further studies establishing the function and utilities of XRCC2 are found in John Hopkins OMIM database record ID 600375, and in sited publications numbered 337-345 listed in the bibliography section hereinbelow, which are also hereby incorporated by reference. APACD (Accession NM_{—}005783) is another VGAM151 host target gene. APACD BINDING SITE is HOST TARGET binding site found in the 3′ untranslated region of mRNA encoded by APACD, corresponding to a HOST TARGET binding site such as BINDING SITE I, BINDING SITE II or BINDING SITE III. Table 2 illustrates the complementarity of the nucleotide sequences of APACD BINDING SITE, designated SEQ ID:1249, to the nucleotide sequence of VGAM151 RNA, herein designated VGAM RNA, also designated SEQ ID:486.

Another function of VGAM151 is therefore inhibition of APACD (Accession NM_{—}005783). Accordingly, utilities of VGAM151 include diagnosis, prevention and treatment of diseases and clinical conditions associated with APACD. SCIN (Accession NM_{—}033128) is another VGAM151 host target gene. SCIN BINDING SITE is HOST TARGET binding site found in the 3′ untranslated region of mRNA encoded by SCIN, corresponding to a HOST TARGET binding site such as BINDING SITE I, BINDING SITE II or BINDING SITE III. Table 2 illustrates the complementarity of the nucleotide sequences of SCIN BINDING SITE, designated SEQ ID:2313, to the nucleotide sequence of VGAM151 RNA, herein designated VGAM RNA, also designated SEQ ID:486.

Another function of VGAM151 is therefore inhibition of SCIN (Accession NM_{—}033128). Accordingly, utilities of VGAM151 include diagnosis, prevention and treatment of diseases and clinical conditions associated with SCIN. Ubiquitin Specific Protease 16 (USP16, Accession NM_{—}006447) is another VGAM151 host target gene. USP16 BINDING SITE is HOST TARGET binding site found in the 3′ untranslated region of mRNA encoded by USP16, corresponding to a HOST TARGET binding site such as BINDING SITE I, BINDING SITE II or BINDING SITE III. Table 2 illustrates the complementarity of the nucleotide sequences of USP16 BINDING SITE, designated SEQ ID:1303, to the nucleotide sequence of VGAM151 RNA, herein designated VGAM RNA, also designated SEQ ID:486.

Another function of VGAM151 is therefore inhibition of Ubiquitin Specific Protease 16 (USP16, Accession NM_{—}006447). Accordingly, utilities of VGAM151 include diagnosis, prevention and treatment of diseases and clinical conditions associated with USP16. LOC115004 (Accession XM_{—}055002) is another VGAM151 host target gene. LOC115004 BINDING SITE is HOST TARGET binding site found in the 3′ untranslated region of mRNA encoded by LOC115004, corresponding to a HOST TARGET binding site such as BINDING SITE I, BINDING SITE II or BINDING SITE III. Table 2 illustrates the complementarity of the nucleotide sequences of LOC115004 BINDING SITE, designated SEQ ID:2970, to the nucleotide sequence of VGAM151 RNA, herein designated VGAM RNA, also designated SEQ ID:486.

Another function of VGAM151 is therefore inhibition of LOC115004 (Accession XM_{—}055002). Accordingly, utilities of VGAM151 include diagnosis, prevention and treatment of diseases and clinical conditions associated with LOC115004. LOC146520 (Accession XM_{—}085492) is another VGAM151 host target gene. LOC146520 BINDING SITE is HOST TARGET binding site found in the 5′ untranslated region of mRNA encoded by LOC146520, corresponding to a HOST TARGET binding site such as BINDING SITE I, BINDING SITE II or BINDING SITE III. Table 2 illustrates the complementarity of the nucleotide sequences of LOC146520 BINDING SITE, designated SEQ ID:3096, to the nucleotide sequence of VGAM151 RNA, herein designated VGAM RNA, also designated SEQ ID:486.

Another function of VGAM151 is therefore inhibition of LOC146520 (Accession XM_{—}085492). Accordingly, utilities of VGAM151 include diagnosis, prevention and treatment of diseases and clinical conditions associated with LOC146520. LOC219333 (Accession XM_{—}167944) is another VGAM151 host target gene. LOC219333 BINDING SITE is HOST TARGET binding site found in the 3′ untranslated region of mRNA encoded by LOC219333, corresponding to a HOST TARGET binding site such as BINDING SITE I, BINDING SITE II or BINDING SITE III. Table 2 illustrates the complementarity of the nucleotide sequences of LOC219333 BINDING SITE, designated SEQ ID:3607, to the nucleotide sequence of VGAM151 RNA, herein designated VGAM RNA, also designated SEQ ID:486.

Another function of VGAM151 is therefore inhibition of LOC219333 (Accession XM_{—}167944). Accordingly, utilities of VGAM151 include diagnosis, prevention and treatment of diseases and clinical conditions associated with LOC219333. FIG. 1 further provides a conceptual description of a novel bioinformatically detected viral gene of the present invention, referred to here as Viral Genomic Address Messenger 152 (VGAM152) viral gene, which modulates expression of respective host target genes thereof, the function and utility of which host target genes is known in the art.

VGAM152 is a novel bioinformatically detected regulatory, non protein coding, viral micro RNA (miRNA) gene. The method by which VGAM152 was detected is described hereinabove with reference to FIGS. 1-8.

VGAM152 gene, herein designated VGAM GENE, is a viral gene contained in the genome of Vaccinia Virus. VGAM152 host target gene, herein designated VGAM HOST TARGET GENE, is a human gene contained in the human genome.

VGAM152 gene encodes a VGAM152 precursor RNA, herein designated VGAM PRECURSOR RNA. Similar to other miRNA genes, and unlike most ordinary genes, VGAM152 precursor RNA does not encode a protein. A nucleotide sequence identical or highly similar to the nucleotide sequence of VGAM152 precursor RNA is designated SEQ ID:138, and is provided hereinbelow with reference to the sequence listing part. Nucleotide sequence SEQ ID:138 is located at position 74425 relative to the genome of Vaccinia Virus.

VGAM152 precursor RNA folds onto itself, forming VGAM152 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM152 folded precursor RNA into VGAM152 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM152 RNA is designated SEQ ID:487, and is provided hereinbelow with reference to the sequence listing part.

VGAM152 host target gene, herein designated VGAM HOST TARGET GENE, encodes a corresponding messenger RNA, VGAM152 host target RNA, herein designated VGAM HOST TARGET RNA. VGAM152 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM152 RNA, herein designated VGAM RNA, binds complementarily to one or more host target binding sites located in untranslated regions of VGAM152 host target RNA, herein designated VGAM HOST TARGET RNA. This complementary binding is due to the fact that the nucleotide sequence of VGAM152 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM152 RNA, herein designated VGAM RNA, may have a different number of host target binding sites in untranslated regions of a VGAM152 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM152 RNA, herein designated VGAM RNA, to host target binding sites on VGAM152 host target RNA, herein designated VGAM HOST TARGET RNA, such as BINDING SITE I, BINDING SITE II and BINDING SITE III, inhibits translation of VGAM152 host target RNA into VGAM152 host target protein, herein designated VGAM HOST TARGET PROTEIN. VGAM host target protein is therefore outlined by a broken line.

It is appreciated that VGAM152 host target gene, herein designated VGAM HOST TARGET GENE, in fact represents a plurality of VGAM152 host target genes. The mRNA of each one of this plurality of VGAM152 host target genes comprises one or more host target binding sites, each having a nucleotide sequence which is at least partly complementary to VGAM152 RNA, herein designated VGAM RNA, and which when bound by VGAM152 RNA causes inhibition of translation of respective one or more VGAM152 host target proteins.

It is further appreciated by one skilled in the art that the mode of translational inhibition illustrated by FIG. 1 with specific reference to translational inhibition exerted by VGAM152 gene, herein designated VGAM GENE, on one or more VGAM152 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM152 is inhibition of expression of host target genes, as part of a novel viral mechanism of attacking a host. Accordingly, utilities of VGAM152 include diagnosis, prevention and treatment of viral infection by Vaccinia Virus. Specific functions, and accordingly utilities, of VGAM152 correlate with, and may be deduced from, the identity of the host target genes which VGAM152 binds and inhibits, and the function of these host target genes, as elaborated hereinbelow.

Nucleotide sequences of the VGAM152 precursor RNA, herein designated VGAM PRECURSOR RNA, and of the ‘diced’ VGAM152 RNA, herein designated VGAM RNA, and a schematic representation of the secondary folding of VGAM152 folded precursor RNA, herein designated VGAM FOLDED PRECURSOR RNA, of VGAM152 are further described hereinbelow with reference to Table 1.

Nucleotide sequences of host target binding sites, such as BINDING SITE-I, BINDING SITE-II and BINDING SITE-III of FIG. 1, found on VGAM152 host target RNA, and schematic representation of the complementarity of each of these host target binding sites to VGAM152 RNA, herein designated VGAM RNA, are described hereinbelow with reference to Table 2.

As mentioned hereinabove with reference to FIG. 1, a function of VGAM152 gene, herein designated VGAM is inhibition of expression of VGAM152 target genes. It is appreciated that specific functions, and accordingly utilities, of VGAM152 correlate with, and may be deduced from, the identity of the target genes which VGAM152 binds and inhibits, and the function of these target genes, as elaborated hereinbelow.

Eukaryotic Translation Initiation Factor 1A (EIF1A, Accession XM_{—}114147) is a VGAM152 host target gene. EIF1A BINDING SITE is HOST TARGET binding site found in the 3′ untranslated region of mRNA encoded by EIF1A, corresponding to a HOST TARGET binding site such as BINDING SITE I, BINDING SITE II or BINDING SITE III. Table 2 illustrates the complementarity of the nucleotide sequences of EIF1A BINDING SITE, designated SEQ ID:3442, to the nucleotide sequence of VGAM152 RNA, herein designated VGAM RNA, also designated SEQ ID:487.

A function of VGAM152 is therefore inhibition of Eukaryotic Translation Initiation Factor 1A (EIF1A, Accession XM_{—}114147), a gene which seems to be required for maximal rate of protein biosynthesis. Accordingly, utilities of VGAM152 include diagnosis, prevention and treatment of diseases and clinical conditions associated with EIF1A. The function of EIF1A has been established by previous studies. The small eukaryotic initiation factor eIF4C is implicated in the translation initiation pathway, where it enhances ribosome dissociation into subunits and stabilizes the binding of the initiator Met-tRNA to 40S ribosomal subunits. Dever et al. (1994) purified rabbit eIF4C and determined its amino acid sequence. By PCR with degenerate primers based on the rabbit eIF4C sequence, they isolated a partial human leukemia cDNA encoding eIF4C. They screened a leukemia cDNA library with the partial cDNA and obtained additional clones corresponding to the entire human eIF4C coding region. The sequence of the predicted 144-amino acid human protein was identical to that of rabbit eIF4C except in a single position. The human protein had an unblocked N-terminal proline, which Dever et al. (1994) stated was consistent with the general pattern of eukaryotic protein processing that removes an initiating methionine when it is followed by a proline. Sequence analysis revealed that eIF4C has a polar structure, with 9 of the first 15 amino acids being basic and 13 of the last 20 amino acids being acidic. The authors suggested that the dipole nature of the protein may allow it to interact with 2 different types of surfaces, perhaps functioning as a bridge between 2 initiation factors or between an initiation factor and the ribosome. Lahn and Page (1997) mapped the EIF1A gene to the X chromosome by analysis of a somatic cell hybrid panel. They designated this gene EIF1AX to distinguish it from the Y-linked homolog, EIF1AY (OMIM Ref. No. 400014). Lahn and Page (1997) determined that the EIF1AX gene escapes X inactivation, and proposed that it should be investigated as a candidate gene for Turner syndrome. See 400010.

Full details of the abovementioned studies are described in the following publications, the disclosure of which are hereby incorporated by reference:
Dever, T. E.; Wei, C.-L.; Benkowski, L. A.; Browning, K.; Merrick, W. C.; Hershey, J. W. B.: Determination of the amino acid sequence of rabbit, human, and wheat germ protein synthesis factor eIF-4C by cloning and chemical sequencing. J. Biol. Chem. 269: 3212-3218, 1994.; and
Lahn, B. T.; Page, D. C.: Functional coherence of the human Y chromosome. Science 278: 675-680, 1997.

Further studies establishing the function and utilities of EIF1A are found in John Hopkins OMIM database record ID 300186, and in sited publications numbered 178 and 2526 listed in the bibliography section hereinbelow, which are also hereby incorporated by reference. Enamelin (ENAM, Accession NM_{—}031889) is another VGAM152 host target gene. ENAM BINDING SITE is HOST TARGET binding site found in the 3′ untranslated region of mRNA encoded by ENAM, corresponding to a HOST TARGET binding site such as BINDING SITE I, BINDING SITE II or BINDING SITE III. Table 2 illustrates the complementarity of the nucleotide sequences of ENAM BINDING SITE, designated SEQ ID:2217, to the nucleotide sequence of VGAM152 RNA, herein designated VGAM RNA, also designated SEQ ID:487.

Another function of VGAM152 is therefore inhibition of Enamelin (ENAM, Accession NM_{—}031889). Accordingly, utilities of VGAM152 include diagnosis, prevention and treatment of diseases and clinical conditions associated with ENAM. V-myc Myelocytomatosis Viral Oncogene Homolog 1, Lung Carcinoma Derived (avian) (MYCL1, Accession NM_{—}005376) is another VGAM152 host target gene. MYCL1 BINDING SITE is HOST TARGET binding site found in the 3′ untranslated region of mRNA encoded by MYCL1, corresponding to a HOST TARGET binding site such as BINDING SITE I, BINDING SITE II or BINDING SITE III. Table 2 illustrates the complementarity of the nucleotide sequences of MYCL1 BINDING SITE, designated SEQ ID:1204, to the nucleotide sequence of VGAM152 RNA, herein designated VGAM RNA, also designated SEQ ID:487.

Another function of VGAM152 is therefore inhibition of V-myc Myelocytomatosis Viral Oncogene Homolog 1, Lung Carcinoma Derived (avian) (MYCL1, Accession NM_{—}005376), a gene which is a Myc-like transcription factor. Accordingly, utilities of VGAM152 include diagnosis, prevention and treatment of diseases and clinical conditions associated with MYCL1. The function of MYCL1 has been established by previous studies. Nau et al. (1985) cloned from DNA of small-cell lung cancer (SCCL) a gene with homology to a small region of both MYC (OMIM Ref. No. 190080) and NMYC (OMIM Ref. No. 164840). By somatic cell hybridization and in situ hybridization, they assigned the gene to 1p32. This LMYC sequence was amplified 10- to 20-fold in the DNA of 4 SCCL lines and of 1 SCCL specimen taken directly from a patient. A restriction polymorphism was found. In heterozygotes, only 1 of the 2 alleles was amplified in any 1 genome. In a linkage map of chromosome 1 prepared by Rouleau et al. (1990), it was concluded that MYCL1 is 17 cM proximal to RH. Kaye et al. (1988) found that the LMYC gene is composed of 3 exons and 2 introns spanning 6.6 kilobases. Several distinct mRNAs were produced in all SCCL cell lines that expressed LMYC. These transcripts were generated from a single gene by alternative splicing of introns 1 and 2 and by use of alternative polyadenylation signals. Comparisons with MYC and NMYC demonstrated multiple discrete regions with extensive homology. Kawashima et al. (1988) concluded that a correlation exists between particular RFLP alleles of the MYCL gene and the occurrence of metastasis of lung cancer to lymph nodes and other or gans. Among lung cancer patients, those with only the L band (10 kb) had few lymph node metastases, whereas patients with either the S band (6 kb) or the S and L bands almost always had lymph node metastases. A similar correlation was found between the presence of the S band and metastases to other organs. The correlation was particularly marked in cases of adenocarcinoma of the lung. By study of DNA from mouse-hamster somatic cell hybrids, Campbell et al. (1989) mapped 2 L-myc loci provisionally to mouse chromosomes 4 and 12. The locus on chromosome 12 may be a pseudogene. When studying chromosome 1p breakpoints in neuroblastoma cell lines using fluorescence in situ hybridization (FISH) with region-specific probes, Van Roy et al. (1995) found evidence for a position of MYCL1 more distal than 1p32. To investigate the discrepancy Speleman et al. (1996) used FISH on high-resolution R-banded chromosomes with a YAC clone for MYCL1 and reassigned the gene to 1p34.3.

Full details of the abovementioned studies are described in the following publications, the disclosure of which are hereby incorporated by reference:
Kawashima, K.; Shikama, H.; Imoto, K.; Izawa, M.; Naruke, T.; Okabayashi, K.; Nishimura, S.: Close correlation between restriction fragment length polymorphism of the LMYC gene and metastasis of human lung cancer to the lymph nodes and other organs. Proc. Nat. Acad. Sci. 85: 2353-2356, 1988.; and
Kaye, F.; Battey, J.; Nau, M.; Brooks, B.; Seifter, E.; De Greve, J.; Birrer, M.; Sausville, E.; Minna, J.: Structure and expression of the human L-myc gene reveal a complex pattern of.

Further studies establishing the function and utilities of MYCL1 are found in John Hopkins OMIM database record ID 164850, and in sited publications numbered 36 and 392-398 listed in the bibliography section hereinbelow, which are also hereby incorporated by reference. Zinc Finger Protein 42 (myeloid-specific retinoic acid-responsive) (ZNF42, Accession NM_{—}003422) is another VGAM152 host target gene. ZNF42 BINDING SITE is HOST TARGET binding site found in the 5′ untranslated region of mRNA encoded by ZNF42, corresponding to a HOST TARGET binding site such as BINDING SITE I, BINDING SITE II or BINDING SITE III. Table 2 illustrates the complementarity of the nucleotide sequences of ZNF42 BINDING SITE, designated SEQ ID:1018, to the nucleotide sequence of VGAM152 RNA, herein designated VGAM RNA, also designated SEQ ID:487.

Another function of VGAM152 is therefore inhibition of Zinc Finger Protein 42 (myeloid-specific retinoic acid-responsive) (ZNF42, Accession NM_{—}003422), a gene which may be one regulator of transcriptional events during hemopoietic development. Accordingly, utilities of VGAM152 include diagnosis, prevention and treatment of diseases and clinical conditions associated with ZNF42. The function of ZNF42 has been established by previous studies. Zinc finger genes encode metal-binding proteins that can act as transcriptional regulators of other genes. In an effort to identify activators of the genetic cascade in hemopoietic differentiation, Hromas et al. (1991) used degenerate synthetic oligonucleotides to the conserved zinc finger histidine-cysteine link to probe a human myeloid lambda gt11 cDNA library. One of the cDNA clones obtained hybridized preferentially to mRNA from myeloid cells. Sequence analysis of the coding region for the gene demonstrated 13 zinc finger regions and a glycine-proline-rich region between the fourth and fifth zinc finger domains. The gene was localized to 19q13.2-q13.4 by chromosomal in situ hybridization, confirmed by hybridization of a labeled probe to dot blots of flow-sorted chromosomes. Chromosome 19 contains other zinc finger genes, e.g., ZFP36 (OMIM Ref. No. 190700), which is located at 19q13.1. The new zinc finger gene, which they designated MZF-1 for ‘myeloid zinc finger,’ was preferentially expressed in myeloid leukemia cell lines, with the highest mRNA levels noted in cells induced to differentiate with retinoic acid. The ZNF42 gene may be a regulator of transcriptional events during hemopoietic development. The myeloid zinc finger gene 1 (MZF1) is a putative transcription factor of the C2H2 zinc finger gene family. Morris et al. (1995) found that MZF1 regulates the CD34 promoter (OMIM Ref. No. 142230) in a tissue-specific manner. They had previously demonstrated MZF-1 binding sites in the promoters of several genes expressed during myeloid differentiation.

Full details of the abovementioned studies are described in the following publications, the disclosure of which are hereby incorporated by reference:
Hromas, R.; Collins, S. J.; Hickstein, D.; Raskind, W.; Deaven, L. L.; O'Hara, P.; Hagen, F. S.; Kaushansky, K.: A retinoic acid-responsive human zinc finger gene, MZF-1, preferentially expressed in myeloid cells. J. Biol. Chem. 266: 14183-14187, 1991.; and
Morris, J. F.; Rauscher, F. J., III; Davis, B.; Klemsz, M.; Xu, D.; Tenen, D.; Hromas, R.: The myeloid zinc finger gene, MZF-1, regulates the CD34 promoter in vitro. Blood 86: 3640-3647.

Further studies establishing the function and utilities of ZNF42 are found in John Hopkins OMIM database record ID 194550, and in sited publications numbered 105-106 listed in the bibliography section hereinbelow, which are also hereby incorporated by reference. FLJ23604 (Accession NM_{—}025064) is another VGAM152 host target gene. FLJ23604 BINDING SITE is HOST TARGET binding site found in the 3′ untranslated region of mRNA encoded by FLJ23604, corresponding to a HOST TARGET binding site such as BINDING SITE I, BINDING SITE II or BINDING SITE III. Table 2 illustrates the complementarity of the nucleotide sequences of FLJ23604 BINDING SITE, designated SEQ ID:2134, to the nucleotide sequence of VGAM152 RNA, herein designated VGAM RNA, also designated SEQ ID:487.

Another function of VGAM152 is therefore inhibition of FLJ23604 (Accession NM_{—}025064). Accordingly, utilities of VGAM152 include diagnosis, prevention and treatment of diseases and clinical conditions associated with FLJ23604. Hairy/enhancer-of-split Related with YRPW Motif 2 (HEY2, Accession NM_{—}012259) is another VGAM152 host target gene. HEY2 BINDING SITE is HOST TARGET binding site found in the 3′ untranslated region of mRNA encoded by HEY2, corresponding to a HOST TARGET binding site such as BINDING SITE I, BINDING SITE II or BINDING SITE III. Table 2 illustrates the complementarity of the nucleotide sequences of HEY2 BINDING SITE, designated SEQ ID: 1419, to the nucleotide sequence of VGAM152 RNA, herein designated VGAM RNA, also designated SEQ ID:487.

Another function of VGAM152 is therefore inhibition of Hairy/enhancer-of-split Related with YRPW Motif 2 (HEY2, Accession NM_{—}012259). Accordingly, utilities of VGAM152 include diagnosis, prevention and treatment of diseases and clinical conditions associated with HEY2. TSC22 (Accession NM_{—}006022) is another VGAM152 host target gene. TSC22 BINDING SITE is HOST TARGET binding site found in the 3′ untranslated region of mRNA encoded by TSC22, corresponding to a HOST TARGET binding site such as BINDING SITE I, BINDING SITE II or BINDING SITE III. Table 2 illustrates the complementarity of the nucleotide sequences of TSC22 BINDING SITE, designated SEQ ID:1265, to the nucleotide sequence of VGAM152 RNA, herein designated VGAM RNA, also designated SEQ ID:487.

Another function of VGAM152 is therefore inhibition of TSC22 (Accession NM_{—}006022). Accordingly, utilities of VGAM152 include diagnosis, prevention and treatment of diseases and clinical conditions associated with TSC22. FIG. 1 further provides a conceptual description of a novel bioinformatically detected viral gene of the present invention, referred to here as Viral Genomic Address Messenger 153 (VGAM153) viral gene, which modulates expression of respective host target genes thereof, the function and utility of which host target genes is known in the art.

VGAM153 is a novel bioinformatically detected regulatory, non protein coding, viral micro RNA (miRNA) gene. The method by which VGAM153 was detected is described hereinabove with reference to FIGS. 1-8.

VGAM153 gene, herein designated VGAM GENE, is a viral gene contained in the genome of Vaccinia Virus. VGAM153 host target gene, herein designated VGAM HOST TARGET GENE, is a human gene contained in the human genome.

VGAM153 gene encodes a VGAM153 precursor RNA, herein designated VGAM PRECURSOR RNA. Similar to other miRNA genes, and unlike most ordinary genes, VGAM153 precursor RNA does not encode a protein. A nucleotide sequence identical or highly similar to the nucleotide sequence of VGAM153 precursor RNA is designated SEQ ID:139, and is provided hereinbelow with reference to the sequence listing part. Nucleotide sequence SEQ ID:139 is located at position 74889 relative to the genome of Vaccinia Virus.

VGAM153 precursor RNA folds onto itself, forming VGAM153 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM153 folded precursor RNA into VGAM153 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM153 RNA is designated SEQ ID:488, and is provided hereinbelow with reference to the sequence listing part.

VGAM153 host target gene, herein designated VGAM HOST TARGET GENE, encodes a corresponding messenger RNA, VGAM153 host target RNA, herein designated VGAM HOST TARGET RNA. VGAM153 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM153 RNA, herein designated VGAM RNA, binds complementarily to one or more host target binding sites located in untranslated regions of VGAM153 host target RNA, herein designated VGAM HOST TARGET RNA. This complementary binding is due to the fact that the nucleotide sequence of VGAM153 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM153 RNA, herein designated VGAM RNA, may have a different number of host target binding sites in untranslated regions of a VGAM153 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM153 RNA, herein designated VGAM RNA, to host target binding sites on VGAM153 host target RNA, herein designated VGAM HOST TARGET RNA, such as BINDING SITE I, BINDING SITE II and BINDING SITE III, inhibits translation of VGAM153 host target RNA into VGAM153 host target protein, herein designated VGAM HOST TARGET PROTEIN. VGAM host target protein is therefore outlined by a broken line.

It is appreciated that VGAM153 host target gene, herein designated VGAM HOST TARGET GENE, in fact represents a plurality of VGAM153 host target genes. The mRNA of each one of this plurality of VGAM153 host target genes comprises one or more host target binding sites, each having a nucleotide sequence which is at least partly complementary to VGAM153 RNA, herein designated VGAM RNA, and which when bound by VGAM153 RNA causes inhibition of translation of respective one or more VGAM153 host target proteins.

It is further appreciated by one skilled in the art that the mode of translational inhibition illustrated by FIG. 1 with specific reference to translational inhibition exerted by VGAM153 gene, herein designated VGAM GENE, on one or more VGAM153 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM153 is inhibition of expression of host target genes, as part of a novel viral mechanism of attacking a host. Accordingly, utilities of VGAM153 include diagnosis, prevention and treatment of viral infection by Vaccinia Virus. Specific functions, and accordingly utilities, of VGAM153 correlate with, and may be deduced from, the identity of the host target genes which VGAM153 binds and inhibits, and the function of these host target genes, as elaborated hereinbelow.

Nucleotide sequences of the VGAM153 precursor RNA, herein designated VGAM PRECURSOR RNA, and of the ‘diced’ VGAM153 RNA, herein designated VGAM RNA, and a schematic representation of the secondary folding of VGAM153 folded precursor RNA, herein designated VGAM FOLDED PRECURSOR RNA, of VGAM153 are further described hereinbelow with reference to Table 1.

Nucleotide sequences of host target binding sites, such as BINDING SITE-I, BINDING SITE-II and BINDING SITE-III of FIG. 1, found on VGAM153 host target RNA, and schematic representation of the complementarity of each of these host target binding sites to VGAM153 RNA, herein designated VGAM RNA, are described hereinbelow with reference to Table 2.

As mentioned hereinabove with reference to FIG. 1, a function of VGAM153 gene, herein designated VGAM is inhibition of expression of VGAM153 target genes. It is appreciated that specific functions, and accordingly utilities, of VGAM153 correlate with, and may be deduced from, the identity of the target genes which VGAM153 binds and inhibits, and the function of these target genes, as elaborated hereinbelow.

FLJ20296 (Accession NM_{—}017750) is a VGAM153 host target gene. FLJ20296 BINDING SITE is HOST TARGET binding site found in the 3′ untranslated region of mRNA encoded by FLJ20296, corresponding to a HOST TARGET binding site such as BINDING SITE I, BINDING SITE II or BINDING SITE III. Table 2 illustrates the complementarity of the nucleotide sequences of FLJ20296 BINDING SITE, designated SEQ ID:1748, to the nucleotide sequence of VGAM153 RNA, herein designated VGAM RNA, also designated SEQ ID:488.

A function of VGAM153 is therefore inhibition of FLJ20296 (Accession NM_{—}017750). Accordingly, utilities of VGAM153 include diagnosis, prevention and treatment of diseases and clinical conditions associated with FLJ20296. RAB22A, Member RAS Oncogene Family (RAB22A, Accession XM_{—}009454) is another VGAM153 host target gene. RAB22A BINDING SITE is HOST TARGET binding site found in the 3′ untranslated region of mRNA encoded by RAB22A, corresponding to a HOST TARGET binding site such as BINDING SITE I, BINDING SITE II or BINDING SITE III. Table 2 illustrates the complementarity of the nucleotide sequences of RAB22A BINDING SITE, designated SEQ ID:2546, to the nucleotide sequence of VGAM153 RNA, herein designated VGAM RNA, also designated SEQ ID:488.

Another function of VGAM153 is therefore inhibition of RAB22A, Member RAS Oncogene Family (RAB22A, Accession XM_{—}009454). Accordingly, utilities of VGAM153 include diagnosis, prevention and treatment of diseases and clinical conditions associated with RAB22A. UPLC1 (Accession NM_{—}017707) is another VGAM153 host target gene. UPLC1 BINDING SITE is HOST TARGET binding site found in the 3′ untranslated region of mRNA encoded by UPLC1, corresponding to a HOST TARGET binding site such as BINDING SITE I, BINDING SITE II or BINDING SITE III. Table 2 illustrates the complementarity of the nucleotide sequences of UPLC1 BINDING SITE, designated SEQ ID:1740, to the nucleotide sequence of VGAM153 RNA, herein designated VGAM RNA, also designated SEQ ID:488.

Another function of VGAM153 is therefore inhibition of UPLC1 (Accession NM_{—}017707). Accordingly, utilities of VGAM153 include diagnosis, prevention and treatment of diseases and clinical conditions associated with UPLC1. LOC122886 (Accession XM_{—}063344) is another VGAM153 host target gene. LOC122886 BINDING SITE is HOST TARGET binding site found in the 3′ untranslated region of mRNA encoded by LOC122886, corresponding to a HOST TARGET binding site such as BINDING SITE I, BINDING SITE II or BINDING SITE III. Table 2 illustrates the complementarity of the nucleotide sequences of LOC122886 BINDING SITE, designated SEQ ID:3028, to the nucleotide sequence of VGAM153 RNA, herein designated VGAM RNA, also designated SEQ ID:488.

Another function of VGAM153 is therefore inhibition of LOC122886 (Accession XM_{—}063344). Accordingly, utilities of VGAM153 include diagnosis, prevention and treatment of diseases and clinical conditions associated with LOC122886. LOC222194 (Accession XM_{—}168443) is another VGAM153 host target gene. LOC222194 BINDING SITE is HOST TARGET binding site found in the 3′ untranslated region of mRNA encoded by LOC222194, corresponding to a HOST TARGET binding site such as BINDING SITE I, BINDING SITE II or BINDING SITE III. Table 2 illustrates the complementarity of the nucleotide sequences of LOC222194 BINDING SITE, designated SEQ ID:3637, to the nucleotide sequence of VGAM153 RNA, herein designated VGAM RNA, also designated SEQ ID:488.

Another function of VGAM153 is therefore inhibition of LOC222194 (Accession XM_{—}168443). Accordingly, utilities of VGAM153 include diagnosis, prevention and treatment of diseases and clinical conditions associated with LOC222194. FIG. 1 further provides a conceptual description of a novel bioinformatically detected viral gene of the present invention, referred to here as Viral Genomic Address Messenger 154 (VGAM154) viral gene, which modulates expression of respective host target genes thereof, the function and utility of which host target genes is known in the art.

VGAM154 is a novel bioinformatically detected regulatory, non protein coding, viral micro RNA (miRNA) gene. The method by which VGAM154 was detected is described hereinabove with reference to FIGS. 1-8.

VGAM154 gene, herein designated VGAM GENE, is a viral gene contained in the genome of Vaccinia Virus. VGAM154 host target gene, herein designated VGAM HOST TARGET GENE, is a human gene contained in the human genome.

VGAM154 gene encodes a VGAM154 precursor RNA, herein designated VGAM PRECURSOR RNA. Similar to other miRNA genes, and unlike most ordinary genes, VGAM154 precursor RNA does not encode a protein. A nucleotide sequence identical or highly similar to the nucleotide sequence of VGAM154 precursor RNA is designated SEQ ID:140, and is provided hereinbelow with reference to the sequence listing part. Nucleotide sequence SEQ ID: 140 is located at position 80970 relative to the genome of Vaccinia Virus.

VGAM154 precursor RNA folds onto itself, forming VGAM154 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM154 folded precursor RNA into VGAM154 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM154 RNA is designated SEQ ID:489, and is provided hereinbelow with reference to the sequence listing part.

VGAM154 host target gene, herein designated VGAM HOST TARGET GENE, encodes a corresponding messenger RNA, VGAM154 host target RNA, herein designated VGAM HOST TARGET RNA. VGAM154 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM154 RNA, herein designated VGAM RNA, binds complementarily to one or more host target binding sites located in untranslated regions of VGAM154 host target RNA, herein designated VGAM HOST TARGET RNA. This complementary binding is due to the fact that the nucleotide sequence of VGAM154 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM154 RNA, herein designated VGAM RNA, may have a different number of host target binding sites in untranslated regions of a VGAM154 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM154 RNA, herein designated VGAM RNA, to host target binding sites on VGAM154 host target RNA, herein designated VGAM HOST TARGET RNA, such as BINDING SITE I, BINDING SITE II and BINDING SITE III, inhibits translation of VGAM154 host target RNA into VGAM154 host target protein, herein designated VGAM HOST TARGET PROTEIN. VGAM host target protein is therefore outlined by a broken line.

It is appreciated that VGAM154 host target gene, herein designated VGAM HOST TARGET GENE, in fact represents a plurality of VGAM154 host target genes. The mRNA of each one of this plurality of VGAM154 host target genes comprises one or more host target binding sites, each having a nucleotide sequence which is at least partly complementary to VGAM154 RNA, herein designated VGAM RNA, and which when bound by VGAM154 RNA causes inhibition of translation of respective one or more VGAM154 host target proteins.

It is further appreciated by one skilled in the art that the mode of translational inhibition illustrated by FIG. 1 with specific reference to translational inhibition exerted by VGAM154 gene, herein designated VGAM GENE, on one or more VGAM154 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM154 is inhibition of expression of host target genes, as part of a novel viral mechanism of attacking a host. Accordingly, utilities of VGAM154 include diagnosis, prevention and treatment of viral infection by Vaccinia Virus. Specific functions, and accordingly utilities, of VGAM154 correlate with, and may be deduced from, the identity of the host target genes which VGAM154 binds and inhibits, and the function of these host target genes, as elaborated hereinbelow.

Nucleotide sequences of the VGAM154 precursor RNA, herein designated VGAM PRECURSOR RNA, and of the ‘diced’ VGAM154 RNA, herein designated VGAM RNA, and a schematic representation of the secondary folding of VGAM154 folded precursor RNA, herein designated VGAM FOLDED PRECURSOR RNA, of VGAM154 are further described hereinbelow with reference to Table 1.

Nucleotide sequences of host target binding sites, such as BINDING SITE-I, BINDING SITE-II and BINDING SITE-III of FIG. 1, found on VGAM154 host target RNA, and schematic representation of the complementarity of each of these host target binding sites to VGAM154 RNA, herein designated VGAM RNA, are described hereinbelow with reference to Table 2.

As mentioned hereinabove with reference to FIG. 1, a function of VGAM154 gene, herein designated VGAM is inhibition of expression of VGAM154 target genes. It is appreciated that specific functions, and accordingly utilities, of VGAM154 correlate with, and may be deduced from, the identity of the target genes which VGAM154 binds and inhibits, and the function of these target genes, as elaborated hereinbelow.

Integrin, Beta 1 (fibronectin receptor, beta polypeptide, antigen CD29 includes MDF2, MSK12) (ITGB1, Accession NM_{—}002211) is a VGAM154 host target gene. ITGB1 BINDING SITE is HOST TARGET binding site found in the 3′ untranslated region of mRNA encoded by ITGB1, corresponding to a HOST TARGET binding site such as BINDING SITE I, BINDING SITE II or BINDING SITE III. Table 2 illustrates the complementarity of the nucleotide sequences of ITGB1 BINDING SITE, designated SEQ ID:909, to the nucleotide sequence of VGAM154 RNA, herein designated VGAM RNA, also designated SEQ ID:489.

A function of VGAM154 is therefore inhibition of Integrin, Beta 1 (fibronectin receptor, beta polypeptide, antigen CD29 includes MDF2, MSK12) (ITGB1, Accession NM_{—}002211), a gene which acts as a fibronectin receptor. Accordingly, utilities of VGAM154 include diagnosis, prevention and treatment of diseases and clinical conditions associated with ITGB1. The function of ITGB1 has been established by previous studies. See 135620. The fibronectin receptors contain a beta subunit that appears to be analogous to band 3 of integrin (Pytela et al., 1986; Johansson et al., 1987). Hynes (1987) proposed that there are 3 subfamilies within the family of human adhesion protein receptor heterodimers based upon the number of different beta subunits. The other 2 subfamilies are the platelet and the endothelial cell heterodimers, which use GP IIIa (OMIM Ref. No. 173470), and the leukocyte heterodimers, which contain a 95,000 Da beta subunit that is homologous to GP IIIa but is clearly a different protein (OMIM Ref. No. 116920). Zhang et al. (1988) examined human-mouse hybrid cells by indirect immunofluorescence with a monoclonal antibody that recognizes the beta subunit of the human fibronectin receptor. Cells that expressed the antigen at their surface were sorted by FACS and karyotyped. The findings, strengthened by isozyme analysis of markers for chromosomes 9 and 10, suggested that the beta subunit is located on 10p By examining the cation dependence of JAM2 (OMIM Ref. No. 606870) adhesion to a T-cell line, Cunningham et al. (2002) identified a manganese-enhanced binding component indicative of integrin involvement. Using neutralizing integrin antibodies, they showed that the manganese-enhanced binding component was due to an interaction between JAM2 and ITGA4/ITGB1. However, the interaction was only enabled following prior adhesion of JAM2 to JAM3 (OMIM Ref. No. 606871). Cunningham et al. (2002) determined that the engagement of all these ligands occurs through a nonacidic residue in an Ig-like fold of JAM2. An inhibitor of ITGA4, TBC772, attenuated the manganese-enhanced binding Animal model experiments lend further support to the function of ITGB1. Graus-Porta et al. (2001) used Cre/Lox-mediated recombination to generate mice with an Itgb1-null allele in the precursors of neurons and glia, thereby inactivating all beta-1-class integrin receptors in the nervous system. The mice died prematurely after birth with severe brain malformations. Using histologic sections of brains at varying ages, Graus-Porta et al. (2001) observed that cortical hemispheres and cerebellar folia fuse, and cortical laminae are perturbed in the knockout mice. These defects result from disorganization of the cortical marginal zone, where Graus-Porta et al. (2001) hypothesized that beta-1-class integrins regulate glial endfeet anchorage, meningeal basement membrane remodeling, and formation of the Cajal-Retzius cell layer. Graus-Porta et al. (2001) concluded that beta-1-class integrins are not essential for neuron-glia interactions and neuronal migration during corticogenesis. They noted that the phenotype of the beta-1-deficient mice resembles pathologic changes observed in human cortical dysplasias, suggesting that defective integrin-mediated signal transduction contributes to the development of some of these diseases

It is appreciated that the abovementioned animal model for ITGB1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Zhang, Y.; Saison, M.; Spaepen, M.; De Strooper, B.; Van Leuven, F.; David, G.; Van den Berghe, H.; Cassiman, J.-J.: Mapping of human fibronectin receptor beta subunit gene to chromosome 10. Somat. Cell Molec. Genet. 14: 99-104, 1988.; and
Graus-Porta, D.; Blaess, S.; Senften, M.; Littlewood-Evans, A.; Damsky, C.; Huang, Z.; Orban, P.; Klein, R.; Schittny, J. C.; Muller, U.: Beta-1-class integrins regulate the development.

Further studies establishing the function and utilities of ITGB1 are found in John Hopkins OMIM database record ID 135630, and in sited publications numbered 762-77 and 796 listed in the bibliography section hereinbelow, which are also hereby incorporated by reference. Rhodopsin (opsin 2, rod pigment) (retinitis pigmentosa 4, autosomal dominant) (RHO, Accession NM_{—}000539) is another VGAM154 host target gene. RHO BINDING SITE is HOST TARGET binding site found in the 3′ untranslated region of mRNA encoded by RHO, corresponding to a HOST TARGET binding site such as BINDING SITE I, BINDING SITE II or BINDING SITE III. Table 2 illustrates the complementarity of the nucleotide sequences of RHO BINDING SITE, designated SEQ ID:754, to the nucleotide sequence of VGAM154 RNA, herein designated VGAM RNA, also designated SEQ ID:489.

Another function of VGAM154 is therefore inhibition of Rhodopsin (opsin 2, rod pigment) (retinitis pigmentosa 4, autosomal dominant) (RHO, Accession NM_{—}000539). Accordingly, utilities of VGAM154 include diagnosis, prevention and treatment of diseases and clinical conditions associated with RHO. X-prolyl Aminopeptidase (aminopeptidase P) 2, Membrane-bound (XPNPEP2, Accession NM_{—}003399) is another VGAM154 host target gene. XPNPEP2 BINDING SITE is HOST TARGET binding site found in the 3′ untranslated region of mRNA encoded by XPNPEP2, corresponding to a HOST TARGET binding site such as BINDING SITE I, BINDING SITE II or BINDING SITE III. Table 2 illustrates the complementarity of the nucleotide sequences of XPNPEP2 BINDING SITE, designated SEQ ID:1015, to the nucleotide sequence of VGAM154 RNA, herein designated VGAM RNA, also designated SEQ ID:489.

Another function of VGAM154 is therefore inhibition of X-prolyl Aminopeptidase (aminopeptidase P) 2, Membrane-bound (XPNPEP2, Accession NM_{—}003399), a gene which is a membrane-associated X-prolyl metallopeptidase. Accordingly, utilities of VGAM154 include diagnosis, prevention and treatment of diseases and clinical conditions associated with XPNPEP2. The function of XPNPEP2 has been established by previous studies. Aminopeptidase P is a widely distributed hydrolase that is specific for N-terminal imido bonds, which are common to several collagen degradation products, neuropeptides, vasoactive peptides, and cytokines. Structurally, the enzyme is a member of the ‘pita bread fold’ family and occurs in mammalian tissues in both soluble and GPI-anchored membrane-bound forms. By RT-PCR using degenerate oligonucleotides based on the pig aminopeptidase P amino acid sequence, and by 5-prime and 3-prime RACE, Venema et al. (1997) isolated human kidney and lung cDNAs encoding XPNPEP2. The deduced XPNPEP2 protein has 673 amino acids and an estimated molecular mass of 75,490 Da. The authors stated that the human and pig XPNPEP2 amino acid sequences show significant evolutionary divergence, with 83% identity; 5 of 6 potential N-glycosylation sites, and 5 of 6 cysteine residues that are potentially involved in disulfide bond formation, are conserved. Northern blot analysis detected a 3.5-kb XPNPEP2 transcript in human kidney, lung, heart, placenta, liver, small intestine, and colon, but not in brain, skeletal muscle, pancreas, spleen, thymus, prostate, testis, ovary, or leukocytes. Based on these results and on RT-PCR studies, Venema et al. (1997) suggested that the membrane-bound form and the soluble form, which is found in brain and leukocytes, are the products of 2 different genes or of alternative splicing of a single primary transcript. Women with balanced translocations between the long arm of the X chromosome and an autosome frequently suffer premature ovarian failure (POF; 311360). Two critical regions for POF which extend from Xq13 to Xq22 and from Xq22 to Xq26 have been identified by cytogenetic studies. To gain insight into the mechanism(s) responsible for ovarian failure in women with X/autosome translocations, Prueitt et al. (2000) molecularly characterized the translocation breakpoints of 9 X chromosomes. They mapped the breakpoints using somatic cell hybrids retaining the derivative autosome and densely spaced markers from the X-chromosome physical map. One of the POF-associated breakpoints in a critical region (Xq25) mapped to a sequenced PAC clone. The translocation disrupts XPNPEP2. XPNPEP2 mRNA was detected in fibroblasts that carried the translocation, suggesting that this gene at least partially escapes X inactivation. Although the physiologic substrates for the enzyme were not known, Prueitt et al. (2000) suggested that XPNPEP2 is a candidate gene for POF.

Full details of the abovementioned studies are described in the following publications, the disclosure of which are hereby incorporated by reference:
Venema, R. C.; Ju, H.; Zou, R.; Venema, V. J.; Ryan, J. W.: Cloning and tissue distribution of human membrane-bound aminopeptidase P. Biochim. Biophys. Acta 1354: 45-48, 1997.; and
Prueitt, R. L.; Ross, J. L.; Zinn, A. R.: Physical mapping of nine Xq translocation breakpoints and identification of XPNPEP2 as a premature ovarian failure candidate gene. Cytogenet.

Further studies establishing the function and utilities of XPNPEP2 are found in John Hopkins OMIM database record ID 300145, and in sited publications numbered 215-217 listed in the bibliography section hereinbelow, which are also hereby incorporated by reference. FLJ12700 (Accession NM_{—}024910) is another VGAM154 host target gene. FLJ12700 BINDING SITE is HOST TARGET binding site found in the 3′ untranslated region of mRNA encoded by FLJ12700, corresponding to a HOST TARGET binding site such as BINDING SITE I, BINDING SITE II or BINDING SITE III. Table 2 illustrates the complementarity of the nucleotide sequences of FLJ12700 BINDING SITE, designated SEQ ID:2110, to the nucleotide sequence of VGAM154 RNA, herein designated VGAM RNA, also designated SEQ ID:489.

Another function of VGAM154 is therefore inhibition of FLJ12700 (Accession NM_{—}024910). Accordingly, utilities of VGAM154 include diagnosis, prevention and treatment of diseases and clinical conditions associated with FLJ12700. FLJ13291 (Accession NM_{—}032178) is another VGAM154 host target gene. FLJ13291 BINDING SITE is HOST TARGET binding site found in the 3′ untranslated region of mRNA encoded by FLJ13291, corresponding to a HOST TARGET binding site such as BINDING SITE I, BINDING SITE II or BINDING SITE III. Table 2 illustrates the complementarity of the nucleotide sequences of FLJ13291 BINDING SITE, designated SEQ ID:2234, to the nucleotide sequence of VGAM154 RNA, herein designated VGAM RNA, also designated SEQ ID:489.

Another function of VGAM154 is therefore inhibition of FLJ13291 (Accession NM_{—}032178). Accordingly, utilities of VGAM154 include diagnosis, prevention and treatment of diseases and clinical conditions associated with FLJ13291. FLJ20811 (Accession XM_{—}040532) is another VGAM154 host target gene. FLJ20811 BINDING SITE is HOST TARGET binding site found in the 5′ untranslated region of mRNA encoded by FLJ20811, corresponding to a HOST TARGET binding site such as BINDING SITE I, BINDING SITE II or BINDING SITE III. Table 2 illustrates the complementarity of the nucleotide sequences of FLJ20811 BINDING SITE, designated SEQ ID:2782, to the nucleotide sequence of VGAM154 RNA, herein designated VGAM RNA, also designated SEQ ID:489.

Another function of VGAM154 is therefore inhibition of FLJ20811 (Accession XM_{—}040532). Accordingly, utilities of VGAM154 include diagnosis, prevention and treatment of diseases and clinical conditions associated with FLJ20811. KIAA1643 (Accession XM_{—}035371) is another VGAM154 host target gene. KIAA1643 BINDING SITE is HOST TARGET binding site found in the 3′ untranslated region of mRNA encoded by KIAA1643, corresponding to a HOST TARGET binding site such as BINDING SITE I, BINDING SITE II or BINDING SITE III. Table 2 illustrates the complementarity of the nucleotide sequences of KIAA1643 BINDING SITE, designated SEQ ID:2693, to the nucleotide sequence of VGAM154 RNA, herein designated VGAM RNA, also designated SEQ ID:489.

Another function of VGAM154 is therefore inhibition of KIAA1643 (Accession XM_{—}035371). Accordingly, utilities of VGAM154 include diagnosis, prevention and treatment of diseases and clinical conditions associated with KIAA1643. KIAA1755 (Accession XM_{—}028810) is another VGAM154 host target gene. KIAA1755 BINDING SITE is HOST TARGET binding site found in the 3′ untranslated region of mRNA encoded by KIAA1755, corresponding to a HOST TARGET binding site such as BINDING SITE I, BINDING SITE II or BINDING SITE III. Table 2 illustrates the complementarity of the nucleotide sequences of KIAA1755 BINDING SITE, designated SEQ ID:2596, to the nucleotide sequence of VGAM154 RNA, herein designated VGAM RNA, also designated SEQ ID:489.

Another function of VGAM154 is therefore inhibition of KIAA1755 (Accession XM_{—}028810). Accordingly, utilities of VGAM154 include diagnosis, prevention and treatment of diseases and clinical conditions associated with KIAA1755. KIAA1762 (Accession XM_{—}033370) is another VGAM154 host target gene. KIAA1762 BINDING SITE is HOST TARGET binding site found in the 3′ untranslated region of mRNA encoded by KIAA1762, corresponding to a HOST TARGET binding site such as BINDING SITE I, BINDING SITE II or BINDING SITE III. Table 2 illustrates the complementarity of the nucleotide sequences of KIAA1762 BINDING SITE, designated SEQ ID:2669, to the nucleotide sequence of VGAM154 RNA, herein designated VGAM RNA, also designated SEQ ID:489.

Another function of VGAM154 is therefore inhibition of KIAA1762 (Accession XM_{—}033370). Accordingly, utilities of VGAM154 include diagnosis, prevention and treatment of diseases and clinical conditions associated with KIAA1762. MGC13033 (Accession NM_{—}031447) is another VGAM154 host target gene. MGC13033 BINDING SITE is HOST TARGET binding site found in the 5′ untranslated region of mRNA encoded by MGC13033, corresponding to a HOST TARGET binding site such as BINDING SITE I, BINDING SITE II or BINDING SITE III. Table 2 illustrates the complementarity of the nucleotide sequences of MGC13033 BINDING SITE, designated SEQ ID:2199, to the nucleotide sequence of VGAM154 RNA, herein designated VGAM RNA, also designated SEQ ID:489.

Another function of VGAM154 is therefore inhibition of MGC13033 (Accession NM_{—}031447). Accordingly, utilities of VGAM154 include diagnosis, prevention and treatment of diseases and clinical conditions associated with MGC13033. Solute Carrier Family 7 (cationic amino acid transporter, y+ system), Member 3 (SLC7A3, Accession NM_{—}032803) is another VGAM154 host target gene. SLC7A3 BINDING SITE is HOST TARGET binding site found in the 5′ untranslated region of mRNA encoded by SLC7A3, corresponding to a HOST TARGET binding site such as BINDING SITE I, BINDING SITE II or BINDING SITE III. Table 2 illustrates the complementarity of the nucleotide sequences of SLC7A3 BINDING SITE, designated SEQ ID:2281, to the nucleotide sequence of VGAM154 RNA, herein designated VGAM RNA, also designated SEQ ID:489.

Another function of VGAM154 is therefore inhibition of Solute Carrier Family 7 (cationic amino acid transporter, y+ system), Member 3 (SLC7A3, Accession NM_{—}032803). Accordingly, utilities of VGAM154 include diagnosis, prevention and treatment of diseases and clinical conditions associated with SLC7A3. LOC112609 (Accession XM_{—}053013) is another VGAM154 host target gene. LOC112609 BINDING SITE is HOST TARGET binding site found in the 3′ untranslated region of mRNA encoded by LOC112609, corresponding to a HOST TARGET binding site such as BINDING SITE I, BINDING SITE II or BINDING SITE III. Table 2 illustrates the complementarity of the nucleotide sequences of LOC112609 BINDING SITE, designated SEQ ID:2960, to the nucleotide sequence of VGAM154 RNA, herein designated VGAM RNA, also designated SEQ ID:489.

Another function of VGAM154 is therefore inhibition of LOC112609 (Accession XM_{—}053013). Accordingly, utilities of VGAM154 include diagnosis, prevention and treatment of diseases and clinical conditions associated with LOC112609. LOC157349 (Accession XM_{—}088298) is another VGAM154 host target gene. LOC157349 BINDING SITE is HOST TARGET binding site found in the 5′ untranslated region of mRNA encoded by LOC157349, corresponding to a HOST TARGET binding site such as BINDING SITE I, BINDING SITE II or BINDING SITE III. Table 2 illustrates the complementarity of the nucleotide sequences of LOC157349 BINDING SITE, designated SEQ ID:3201, to the nucleotide sequence of VGAM154 RNA, herein designated VGAM RNA, also designated SEQ ID:489.

Another function of VGAM154 is therefore inhibition of LOC157349 (Accession XM_{—}088298). Accordingly, utilities of VGAM154 include diagnosis, prevention and treatment of diseases and clinical conditions associated with LOC157349. LOC90625 (Accession XM_{—}033004) is another VGAM154 host target gene. LOC90625 BINDING SITE is HOST TARGET binding site found in the 3′ untranslated region of mRNA encoded by LOC90625, corresponding to a HOST TARGET binding site such as BINDING SITE I, BINDING SITE II or BINDING SITE III. Table 2 illustrates the complementarity of the nucleotide sequences of LOC90625 BINDING SITE, designated SEQ ID:2662, to the nucleotide sequence of VGAM154 RNA, herein designated VGAM RNA, also designated SEQ ID:489.

Another function of VGAM154 is therefore inhibition of LOC90625 (Accession XM_{—}033004). Accordingly, utilities of VGAM154 include diagnosis, prevention and treatment of diseases and clinical conditions associated with LOC90625. FIG. 1 further provides a conceptual description of a novel bioinformatically detected viral gene of the present invention, referred to here as Viral Genomic Address Messenger 155 (VGAM155) viral gene, which modulates expression of respective host target genes thereof, the function and utility of which host target genes is known in the art.

VGAM155 is a novel bioinformatically detected regulatory, non protein coding, viral micro RNA (miRNA) gene. The method by which VGAM155 was detected is described hereinabove with reference to FIGS. 1-8.

VGAM155 gene, herein designated VGAM GENE, is a viral gene contained in the genome of Vaccinia Virus. VGAM155 host target gene, herein designated VGAM HOST TARGET GENE, is a human gene contained in the human genome.

VGAM155 gene encodes a VGAM155 precursor RNA, herein designated VGAM PRECURSOR RNA. Similar to other miRNA genes, and unlike most ordinary genes, VGAM155 precursor RNA does not encode a protein. A nucleotide sequence identical or highly similar to the nucleotide sequence of VGAM155 precursor RNA is designated SEQ ID:141, and is provided hereinbelow with reference to the sequence listing part. Nucleotide sequence SEQ ID: 141 is located at position 80298 relative to the genome of Vaccinia Virus.

VGAM155 precursor RNA folds onto itself, forming VGAM155 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM155 folded precursor RNA into VGAM155 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM155 RNA is designated SEQ ID:490, and is provided hereinbelow with reference to the sequence listing part.

VGAM155 host target gene, herein designated VGAM HOST TARGET GENE, encodes a corresponding messenger RNA, VGAM155 host target RNA, herein designated VGAM HOST TARGET RNA. VGAM155 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM155 RNA, herein designated VGAM RNA, binds complementarily to one or more host target binding sites located in untranslated regions of VGAM155 host target RNA, herein designated VGAM HOST TARGET RNA. This complementary binding is due to the fact that the nucleotide sequence of VGAM155 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM155 RNA, herein designated VGAM RNA, may have a different number of host target binding sites in untranslated regions of a VGAM155 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM155 RNA, herein designated VGAM RNA, to host target binding sites on VGAM155 host target RNA, herein designated VGAM HOST TARGET RNA, such as BINDING SITE I, BINDING SITE II and BINDING SITE III, inhibits translation of VGAM155 host target RNA into VGAM155 host target protein, herein designated VGAM HOST TARGET PROTEIN. VGAM host target protein is therefore outlined by a broken line.

It is appreciated that VGAM155 host target gene, herein designated VGAM HOST TARGET GENE, in fact represents a plurality of VGAM155 host target genes. The mRNA of each one of this plurality of VGAM155 host target genes comprises one or more host target binding sites, each having a nucleotide sequence which is at least partly complementary to VGAM155 RNA, herein designated VGAM RNA, and which when bound by VGAM155 RNA causes inhibition of translation of respective one or more VGAM155 host target proteins.

It is further appreciated by one skilled in the art that the mode of translational inhibition illustrated by FIG. 1 with specific reference to translational inhibition exerted by VGAM155 gene, herein designated VGAM GENE, on one or more VGAM155 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM155 is inhibition of expression of host target genes, as part of a novel viral mechanism of attacking a host. Accordingly, utilities of VGAM155 include diagnosis, prevention and treatment of viral infection by Vaccinia Virus. Specific functions, and accordingly utilities, of VGAM155 correlate with, and may be deduced from, the identity of the host target genes which VGAM155 binds and inhibits, and the function of these host target genes, as elaborated hereinbelow.

Nucleotide sequences of the VGAM155 precursor RNA, herein designated VGAM PRECURSOR RNA, and of the ‘diced’ VGAM155 RNA, herein designated VGAM RNA, and a schematic representation of the secondary folding of VGAM155 folded precursor RNA, herein designated VGAM FOLDED PRECURSOR RNA, of VGAM155 are further described hereinbelow with reference to Table 1.

Nucleotide sequences of host target binding sites, such as BINDING SITE-I, BINDING SITE-II and BINDING SITE-III of FIG. 1, found on VGAM155 host target RNA, and schematic representation of the complementarity of each of these host target binding sites to VGAM155 RNA, herein designated VGAM RNA, are described hereinbelow with reference to Table 2.

As mentioned hereinabove with reference to FIG. 1, a function of VGAM155 gene, herein designated VGAM is inhibition of expression of VGAM155 target genes. It is appreciated that specific functions, and accordingly utilities, of VGAM155 correlate with, and may be deduced from, the identity of the target genes which VGAM155 binds and inhibits, and the function of these target genes, as elaborated hereinbelow.

Mannosyl (alpha-1,3-)-glycoprotein Beta-1,4-N-acetylglucosaminyltransferase, Isoenzyme B (MGAT4B, Accession NM_{—}054013) is a VGAM155 host target gene. MGAT4B BINDING SITE is HOST TARGET binding site found in the 5′ untranslated region of mRNA encoded by MGAT4B, corresponding to a HOST TARGET binding site such as BINDING SITE I, BINDING SITE II or BINDING SITE III. Table 2 illustrates the complementarity of the nucleotide sequences of MGAT4B BINDING SITE, designated SEQ ID:2360, to the nucleotide sequence of VGAM155 RNA, herein designated VGAM RNA, also designated SEQ ID:490.

A function of VGAM155 is therefore inhibition of Mannosyl (alpha-1,3-)-glycoprotein Beta-1,4-N-acetylglucosaminyltransferase, Isoenzyme B (MGAT4B, Accession NM_{—}054013). Accordingly, utilities of VGAM155 include diagnosis, prevention and treatment of diseases and clinical conditions associated with MGAT4B. Protein Kinase, CAMP-dependent, Regulatory, Type II, Alpha (PRKAR2A, Accession NM_{—}004157) is another VGAM155 host target gene. PRKAR2A BINDING SITE is HOST TARGET binding site found in the 3′ untranslated region of mRNA encoded by PRKAR2A, corresponding to a HOST TARGET binding site such as BINDING SITE I, BINDING SITE II or BINDING SITE III. Table 2 illustrates the complementarity of the nucleotide sequences of PRKAR2A BINDING SITE, designated SEQ ID: 1091, to the nucleotide sequence of VGAM155 RNA, herein designated VGAM RNA, also designated SEQ ID:490.

Another function of VGAM155 is therefore inhibition of Protein Kinase, CAMP-dependent, Regulatory, Type II, Alpha (PRKAR2A, Accession NM_{—}004157), a gene which mediates membrane association by binding to anchoring proteins, including the map2 kinase. Accordingly, utilities of VGAM155 include diagnosis, prevention and treatment of diseases and clinical conditions associated with PRKAR2A. The function of PRKAR2A has been established by previous studies. See 188830. Phosphorylation by cAMP-dependent protein kinases is essential for sperm motility. A cAMP-dependent protein kinase is bound to sperm flagella by a regulatory subunit (RII). Oyen et al. (1989) observed high testis-specific expression of a human homolog to the rat RII-alpha mRNA induced in haploid germ cells. They cloned a human cDNA that encodes a 404-amino acid polypeptide with a region (amino acids 45-75) divergent from that of the previously published mouse and rat sequences. By PCR and Southern blot analysis of somatic cell hybrid mapping panels and by radiation hybrid analysis, Tasken et al. (1998) mapped the PRKAR2A gene to chromosome 3p21.3-p21.2.

Full details of the abovementioned studies are described in the following publications, the disclosure of which are hereby incorporated by reference:
Oyen, O.; Myklebust, F.; Scott, J. D.; Hansson, V.; Jahnsen, T.: Human testis cDNA for the regulatory subunit RII alpha of cAMP-dependent protein kinase encodes an alternate amino-terminal region. FEBS Lett. 246: 57-64, 1989.; and
Tasken, K.; Naylor, S. L.; Solberg, R.; Jahnsen, T.: Mapping of the gene encoding the regulatory subunit RII-alpha of cAMP-dependent protein kinase (locus PRKAR2A) to human chromosome r.

Further studies establishing the function and utilities of PRKAR2A are found in John Hopkins OMIM database record ID 176910, and in sited publications numbered 279-280 listed in the bibliography section hereinbelow, which are also hereby incorporated by reference. Stearoyl-CoA Desaturase (delta-9-desaturase) (SCD, Accession NM_{—}005063) is another VGAM155 host target gene. SCD BINDING SITE is HOST TARGET binding site found in the 3′ untranslated region of mRNA encoded by SCD, corresponding to a HOST TARGET binding site such as BINDING SITE I, BINDING SITE II or BINDING SITE III. Table 2 illustrates the complementarity of the nucleotide sequences of SCD BINDING SITE, designated SEQ ID:1177, to the nucleotide sequence of VGAM155 RNA, herein designated VGAM RNA, also designated SEQ ID:490.

Another function of VGAM155 is therefore inhibition of Stearoyl-CoA Desaturase (delta-9-desaturase) (SCD, Accession NM_{—}005063), a gene which functions in the synthesis of unsaturated fatty acids. Accordingly, utilities of VGAM155 include diagnosis, prevention and treatment of diseases and clinical conditions associated with SCD. The function of SCD has been established by previous studies. Stearoyl-CoA desaturase (SCD; EC 1.14.99.5) is an iron-containing enzyme that catalyzes a rate-limiting step in the synthesis of unsaturated fatty acids. The principal product of SCD is oleic acid, which is formed by desaturation of stearic acid. The ratio of stearic acid to oleic acid has been implicated in the regulation of cell growth and differentiation through effects on cell-membrane fluidity and signal transduction (Zhang et al. (1999)). Thiede et al. (1986) isolated cDNAs encoding rat SCD. By RT-PCR of adipose tissue RNA with primers based on the sequence of rat SCD, L1 et al. (1994) isolated a partial human SCD cDNA. Using RNase protection assays, the authors found that human SCD was expressed at higher levels in colon and esophageal carcinomas than in the counterpart normal tissues. Animal model experiments lend further support to the function of SCD. SCD is a central lipogenic enzyme catalyzing the synthesis of monounsaturated fatty acids, mainly oleate (C18:1) and palmitoleate (C16:1), which are components of membrane phospholipids, triglycerides, wax esters, and cholesterol esters. Several SCD isoforms (SCD1, -2, and -3) exist in the mouse. Ntambi et al. (2002) showed that mice with a targeted disruption of the SCD1 isoform had reduced body adiposity, increased insulin (OMIM Ref. No. 176730) sensitivity, and resistance to diet-induced weight gain. The protection from obesity involved increased energy expenditure and increased oxygen consumption. Compared with wildtype mice, the SCD1 −/− mice had increased levels of plasma ketone bodies but reduced levels of plasma insulin and leptin. In these homozygous null mice, the expression of several genes of lipid oxidation was up-regulated, whereas lipid synthesis genes were downregulated. These observations suggested that a consequence of SCD1 deficiency is an activation of lipid oxidation in addition to reduced triglyceride synthesis and storage.

It is appreciated that the abovementioned animal model for SCD is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ntambi, J. M.; Miyazaki, M.; Stoehr, J. P.; Lan, H.; Kendziorski, C. M.; Yandell, B. S.; Song, Y.; Cohen, P.; Friedman, J. M.; Attie, A. D.: Loss of stearoyl-CoA desaturase-1 function protects mice against adiposity. Proc. Nat. Acad. Sci. 99: 11482-11486, 2002.; and
Zhang, L.; Ge, L.; Parimoo, S.; Sterm, K.; Prouty, S. M.: Human stearoyl-CoA desaturase: alternative transcripts generated from a single gene by usage of tandem polyadenylation sites.

Further studies establishing the function and utilities of SCD are found in John Hopkins OMIM database record ID 604031, and in sited publications numbered 72 and 1750-1754 listed in the bibliography section hereinbelow, which are also hereby incorporated by reference. DKFZp761H079 (Accession NM_{—}144996) is another VGAM155 host target gene. DKFZp761H079 BINDING SITE is HOST TARGET binding site found in the 3′ untranslated region of mRNA encoded by DKFZp761H079, corresponding to a HOST TARGET binding site such as BINDING SITE I, BINDING SITE II or BINDING SITE III. Table 2 illustrates the complementarity of the nucleotide sequences of DKFZp761H079 BINDING SITE, designated SEQ ID:2506, to the nucleotide sequence of VGAM155 RNA, herein designated VGAM RNA, also designated SEQ ID:490.

Another function of VGAM155 is therefore inhibition of DKFZp761H079 (Accession NM_{—}144996). Accordingly, utilities of VGAM155 include diagnosis, prevention and treatment of diseases and clinical conditions associated with DKFZp761H079. KIAA0417 (Accession XM_{—}048898) is another VGAM155 host target gene. KIAA0417 BINDING SITE is HOST TARGET binding site found in the 3′ untranslated region of mRNA encoded by KIAA0417, corresponding to a HOST TARGET binding site such as BINDING SITE I, BINDING SITE II or BINDING SITE III. Table 2 illustrates the complementarity of the nucleotide sequences of KIAA0417 BINDING SITE, designated SEQ ID:2910, to the nucleotide sequence of VGAM155 RNA, herein designated VGAM RNA, also designated SEQ ID:490.

Another function of VGAM155 is therefore inhibition of KIAA0417 (Accession XM_{—}048898). Accordingly, utilities of VGAM155 include diagnosis, prevention and treatment of diseases and clinical conditions associated with KIAA0417. LPS-responsive Vesicle Trafficking, Beach and Anchor Containing (LRBA, Accession NM_{—}006726) is another VGAM155 host target gene. LRBA BINDING SITE is HOST TARGET binding site found in the 3′ untranslated region of mRNA encoded by LRBA, corresponding to a HOST TARGET binding site such as BINDING SITE I, BINDING SITE II or BINDING SITE III. Table 2 illustrates the complementarity of the nucleotide sequences of LRBA BINDING SITE, designated SEQ ID:1327, to the nucleotide sequence of VGAM155 RNA, herein designated VGAM RNA, also designated SEQ ID:490.

Another function of VGAM155 is therefore inhibition of LPS-responsive Vesicle Trafficking, Beach and Anchor Containing (LRBA, Accession NM_{—}006726). Accordingly, utilities of VGAM155 include diagnosis, prevention and treatment of diseases and clinical conditions associated with LRBA. Reserved (SYAP1, Accession NM_{—}032796) is another VGAM155 host target gene. SYAP1 BINDING SITE is HOST TARGET binding site found in the 3′ untranslated region of mRNA encoded by SYAP1, corresponding to a HOST TARGET binding site such as BINDING SITE I, BINDING SITE II or BINDING SITE III. Table 2 illustrates the complementarity of the nucleotide sequences of SYAP1 BINDING SITE, designated SEQ ID:2279, to the nucleotide sequence of VGAM155 RNA, herein designated VGAM RNA, also designated SEQ ID:490.

Another function of VGAM155 is therefore inhibition of Reserved (SYAP1, Accession NM_{—}032796). Accordingly, utilities of VGAM155 include diagnosis, prevention and treatment of diseases and clinical conditions associated with SYAP1. LOC143188 (Accession XM_{—}096387) is another VGAM155 host target gene. LOC143188 BINDING SITE is HOST TARGET binding site found in the 5′ untranslated region of mRNA encoded by LOC143188, corresponding to a HOST TARGET binding site such as BINDING SITE I, BINDING SITE II or BINDING SITE III. Table 2 illustrates the complementarity of the nucleotide sequences of LOC143188 BINDING SITE, designated SEQ ID:3264, to the nucleotide sequence of VGAM155 RNA, herein designated VGAM RNA, also designated SEQ ID:490.

Another function of VGAM155 is therefore inhibition of LOC143188 (Accession XM_{—}096387). Accordingly, utilities of VGAM155 include diagnosis, prevention and treatment of diseases and clinical conditions associated with LOC143188. LOC157292 (Accession XM_{—}098740) is another VGAM155 host target gene. LOC157292 BINDING SITE is HOST TARGET binding site found in the 3′ untranslated region of mRNA encoded by LOC157292, corresponding to a HOST TARGET binding site such as BINDING SITE I, BINDING SITE II or BINDING SITE III. Table 2 illustrates the complementarity of the nucleotide sequences of LOC157292 BINDING SITE, designated SEQ ID:3378, to the nucleotide sequence of VGAM155 RNA, herein designated VGAM RNA, also designated SEQ ID:490.

Another function of VGAM155 is therefore inhibition of LOC157292 (Accession XM_{—}098740). Accordingly, utilities of VGAM155 include diagnosis, prevention and treatment of diseases and clinical conditions associated with LOC157292. FIG. 1 further provides a conceptual description of a novel bioinformatically detected viral gene of the present invention, referred to here as Viral Genomic Address Messenger 156 (VGAM156) viral gene, which modulates expression of respective host target genes thereof, the function and utility of which host target genes is known in the art.

VGAM156 is a novel bioinformatically detected regulatory, non protein coding, viral micro RNA (miRNA) gene. The method by which VGAM156 was detected is described hereinabove with reference to FIGS. 1-8.

VGAM156 gene, herein designated VGAM GENE, is a viral gene contained in the genome of Vaccinia Virus. VGAM156 host target gene, herein designated VGAM HOST TARGET GENE, is a human gene contained in the human genome.

VGAM156 gene encodes a VGAM156 precursor RNA, herein designated VGAM PRECURSOR RNA. Similar to other miRNA genes, and unlike most ordinary genes, VGAM156 precursor RNA does not encode a protein. A nucleotide sequence identical or highly similar to the nucleotide sequence of VGAM156 precursor RNA is designated SEQ ID:142, and is provided hereinbelow with reference to the sequence listing part. Nucleotide sequence SEQ ID: 142 is located at position 79822 relative to the genome of Vaccinia Virus.

VGAM156 precursor RNA folds onto itself, forming VGAM156 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM156 folded precursor RNA into VGAM156 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM156 RNA is designated SEQ ID:491, and is provided hereinbelow with reference to the sequence listing part.

VGAM156 host target gene, herein designated VGAM HOST TARGET GENE, encodes a corresponding messenger RNA, VGAM156 host target RNA, herein designated VGAM HOST TARGET RNA. VGAM156 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM156 RNA, herein designated VGAM RNA, binds complementarily to one or more host target binding sites located in untranslated regions of VGAM156 host target RNA, herein designated VGAM HOST TARGET RNA. This complementary binding is due to the fact that the nucleotide sequence of VGAM156 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM156 RNA, herein designated VGAM RNA, may have a different number of host target binding sites in untranslated regions of a VGAM156 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM156 RNA, herein designated VGAM RNA, to host target binding sites on VGAM156 host target RNA, herein designated VGAM HOST TARGET RNA, such as BINDING SITE I, BINDING SITE II and BINDING SITE III, inhibits translation of VGAM156 host target RNA into VGAM156 host target protein, herein designated VGAM HOST TARGET PROTEIN. VGAM host target protein is therefore outlined by a broken line.

It is appreciated that VGAM156 host target gene, herein designated VGAM HOST TARGET GENE, in fact represents a plurality of VGAM156 host target genes. The mRNA of each one of this plurality of VGAM156 host target genes comprises one or more host target binding sites, each having a nucleotide sequence which is at least partly complementary to VGAM156 RNA, herein designated VGAM RNA, and which when bound by VGAM156 RNA causes inhibition of translation of respective one or more VGAM156 host target proteins.

It is further appreciated by one skilled in the art that the mode of translational inhibition illustrated by FIG. 1 with specific reference to translational inhibition exerted by VGAM156 gene, herein designated VGAM GENE, on one or more VGAM156 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM156 is inhibition of expression of host target genes, as part of a novel viral mechanism of attacking a host. Accordingly, utilities of VGAM156 include diagnosis, prevention and treatment of viral infection by Vaccinia Virus. Specific functions, and accordingly utilities, of VGAM156 correlate with, and may be deduced from, the identity of the host target genes which VGAM156 binds and inhibits, and the function of these host target genes, as elaborated hereinbelow.

Nucleotide sequences of the VGAM156 precursor RNA, herein designated VGAM PRECURSOR RNA, and of the ‘diced’ VGAM156 RNA, herein designated VGAM RNA, and a schematic representation of the secondary folding of VGAM156 folded precursor RNA, herein designated VGAM FOLDED PRECURSOR RNA, of VGAM156 are further described hereinbelow with reference to Table 1.

Nucleotide sequences of host target binding sites, such as BINDING SITE-I, BINDING SITE-II and BINDING SITE-III of FIG. 1, found on VGAM156 host target RNA, and schematic representation of the complementarity of each of these host target binding sites to VGAM156 RNA, herein designated VGAM RNA, are described hereinbelow with reference to Table 2.

As mentioned hereinabove with reference to FIG. 1, a function of VGAM156 gene, herein designated VGAM is inhibition of expression of VGAM156 target genes. It is appreciated that specific functions, and accordingly utilities, of VGAM156 correlate with, and may be deduced from, the identity of the target genes which VGAM156 binds and inhibits, and the function of these target genes, as elaborated hereinbelow.

Chromosome 18 Open Reading Frame 1 (C18orf1, Accession XM_{—}012723) is a VGAM156 host target gene. C18orf1 BINDING SITE is HOST TARGET binding site found in the 3′ untranslated region of mRNA encoded by C18orf1, corresponding to a HOST TARGET binding site such as BINDING SITE I, BINDING SITE II or BINDING SITE III. Table 2 illustrates the complementarity of the nucleotide sequences of C18orf1 BINDING SITE, designated SEQ ID:2561, to the nucleotide sequence of VGAM156 RNA, herein designated VGAM RNA, also designated SEQ ID:491.

A function of VGAM156 is therefore inhibition of Chromosome 18 Open Reading Frame 1 (C18orf1, Accession XM_{—}012723), a gene which displays selective expression, regulated spatially and temporally. Accordingly, utilities of VGAM156 include diagnosis, prevention and treatment of diseases and clinical conditions associated with C18orf1. The function of C18orf1 has been established by previous studies. By cDNA selection, Yoshikawa et al. (1997) isolated several brain-derived transcripts on chromosome 18, including C18ORF1, which maps to 18p11.2. Yoshikawa et al. (1998) further characterized C18ORF1 and identified 2 major classes of transcripts distinguished by their unique upstream regions, with the beta variants representing N-terminal-truncated versions of the alpha isoforms. The alpha-specific exons 1 to 3 are missing in beta; instead, the downstream sequence of intron C is present in the mature mRNA (exon 4a), which combines with exon 4b to create the upstream exon for beta. The differential splicing of exon 5 leads to the expression of 2 alpha and 2 beta subclasses. Alpha-1 and beta-1 share identical sequences with alpha-2 and beta-2, respectively, except for the loss of exon 5 in alpha-2 and beta-2. The differences between the alpha and the beta classes of transcripts predict 2 potentially distinct promoters for the C18ORF1 gene. Yoshikawa et al. (1998) also presented evidence of RNA editing in the 5-prime untranslated region of the beta-2 variant. Yoshikawa et al. (1998) demonstrated that the C18ORF1 transcripts display selective expression, regulated spatially and temporally. The cortical and subcortical structures of brain appear to express high levels of alpha, in both fetal and adult stages. The major 9-kb transcript of C18ORF1, detectable in early development in kidney, liver, and lung, is indiscernible in these tissues in the adult. The expression profile displayed by beta is more complex. Northern blots generated from adult tissues are devoid of hybridization signals with beta but fetal tissues exhibit a 4-kb transcript. In contrast, all subcortical areas of adult brain show relatively intense signals at 6.8 kb, implying a different mechanism of transcript processing in adult brain structures. Northern blot analysis also detected several minor bands in the alpha and beta isoforms.

Full details of the abovementioned studies are described in the following publications, the disclosure of which are hereby incorporated by reference:
Yoshikawa, T.; Sanders, A. R.; Esterling, L. E.; Detera-Wadleigh, S. D.: Multiple transcriptional variants and RNA editing in C18orf1, a novel gene with LDLRA and transmembrane domains on 18p11.2. Genomics 47: 246-257, 1998.; and
Yoshikawa, T.; Sanders, A. R.; Esterling, L. E.; Overharser, J.; Garnes, J. A.; Lennon, G.; Grewal, R.; Detera-Wadleigh, S. D.: Isolation of chromosome 18-specific brain transcripts as.

Further studies establishing the function and utilities of C18orf1 are found in John Hopkins OMIM database record ID 606571, and in sited publications numbered 1407-1408 listed in the bibliography section hereinbelow, which are also hereby incorporated by reference. Crystallin, Zeta (quinone reductase) (CRYZ, Accession NM_{—}001889) is another VGAM156 host target gene. CRYZ BINDING SITE is HOST TARGET binding site found in the 3′ untranslated region of mRNA encoded by CRYZ, corresponding to a HOST TARGET binding site such as BINDING SITE I, BINDING SITE II or BINDING SITE III. Table 2 illustrates the complementarity of the nucleotide sequences of CRYZ BINDING SITE, designated SEQ ID:866, to the nucleotide sequence of VGAM156 RNA, herein designated VGAM RNA, also designated SEQ ID:491.

Another function of VGAM156 is therefore inhibition of Crystallin, Zeta (quinone reductase) (CRYZ, Accession NM_{—}001889), a gene which may act in the detoxification of xenobiotics. Accordingly, utilities of VGAM156 include diagnosis, prevention and treatment of diseases and clinical conditions associated with CRYZ. The function of CRYZ has been established by previous studies. In addition to the alpha, beta, and gamma crystallin families, which are present in the ocular lenses of all vertebrates, a number of other crystallins have been found to be present in high amounts in lenses from phylogenetically restricted groups. Most of these ‘taxon-specific’ crystallins are pyridine nucleotide-dependent oxidoreductases that are also present at enzymatic levels in nonlenticular tissues. The acquisition of this new function as a lens crystallin generally occurs without gene duplication and apparently without affecting the catalytic role of the enzyme. Zeta-crystallin/quinone reductase was initially described as a major protein in the lens of the guinea pig (Huang et al., 1987), in which a mutation in the gene is associated with hereditary cataracts (Rodriguez et al., 1992). It was later found to be also present in high amounts in the lens of camels (Garland et al., 1991) and at enzymatic levels in a number of nonlenticular tissues of various species. In the lens of guinea pigs and camels, it comprises about 10% of the total soluble protein. Gonzalez et al. (1994) isolated and characterized the human zeta-crystallin gene and its processed pseudogene. The functional gene is composed of 9 exons and spans about 20 kb. The 5-prime flanking region of the gene is rich in G and C (58%) and lacks TATA and CAAT boxes. Previous analysis of the guinea pig gene revealed the presence of 2 different promoters, one responsible for the high lens-specific expression and the other for expression at the enzymatic level in numerous tissues. A comparative analysis with the guinea pig gene showed that a region of approximately 2.5 kb that includes the promoter responsible for the high expression in the lens in the guinea pig is not present in the human gene By Southern analysis of human/mouse somatic cell hybrids, Heinzmann et al. (1994) assigned the CRYZ gene to human chromosome 1 and regionalized the assignment to 1p31-p22 by fluorescence in situ hybridization. They also identified 5 RFLPs

Full details of the abovementioned studies are described in the following publications, the disclosure of which are hereby incorporated by reference:
Gonzalez, P.; Rao, P. V.; Zigler, J. S., Jr.: Organization of the human zeta-crystallin/quinone reductase gene (CRYZ). Genomics 21: 317-324, 1994.; and
Heinzmann, C.; Kojis, T. L.; Gonzalez, P.; Rao, P. V.; Zigler, J. S., Jr.; Polymeropoulos, M. H.; Klisak, I.; Sparkes, R. S.; Mohandas, T.; Bateman, J. B.: Assignment of the zeta-crysta.

Further studies establishing the function and utilities of CRYZ are found in John Hopkins OMIM database record ID 123691, and in sited publications numbered 2676-2680 listed in the bibliography section hereinbelow, which are also hereby incorporated by reference. Transient Receptor Potential Cation Channel, Sub family C, Member 5 (TRPC5, Accession NM_{—}012471) is another VGAM156 host target gene. TRPC5 BINDING SITE is HOST TARGET binding site found in the 3′ untranslated region of mRNA encoded by TRPC5, corresponding to a HOST TARGET binding site such as BINDING SITE I, BINDING SITE II or BINDING SITE III. Table 2 illustrates the complementarity of the nucleotide sequences of TRPC5 BINDING SITE, designated SEQ ID: 1434, to the nucleotide sequence of VGAM156 RNA, herein designated VGAM RNA, also designated SEQ ID:491.

Another function of VGAM156 is therefore inhibition of Transient Receptor Potential Cation Channel, Sub family C, Member 5 (TRPC5, Accession NM_{—}012471). Accordingly, utilities of VGAM156 include diagnosis, prevention and treatment of diseases and clinical conditions associated with TRPC5. DAP13 (Accession NM_{—}018838) is another VGAM156 host target gene. DAP13 BINDING SITE is HOST TARGET binding site found in the 3′ untranslated region of mRNA encoded by DAP13, corresponding to a HOST TARGET binding site such as BINDING SITE I, BINDING SITE II or BINDING SITE III. Table 2 illustrates the complementarity of the nucleotide sequences of DAP13 BINDING SITE, designated SEQ ID:1862, to the nucleotide sequence of VGAM156 RNA, herein designated VGAM RNA, also designated SEQ ID:491.

Another function of VGAM156 is therefore inhibition of DAP13 (Accession NM_{—}018838). Accordingly, utilities of VGAM156 include diagnosis, prevention and treatment of diseases and clinical conditions associated with DAP13. DKFZP56400463 (Accession NM_{—}014156) is another VGAM156 host target gene. DKFZP56400463 BINDING SITE is HOST TARGET binding site found in the 3′ untranslated region of mRNA encoded by DKFZP56400463, corresponding to a HOST TARGET binding site such as BINDING SITE I, BINDING SITE II or BINDING SITE III. Table 2 illustrates the complementarity of the nucleotide sequences of DKFZP56400463 BINDING SITE, designated SEQ ID: 1481, to the nucleotide sequence of VGAM156 RNA, herein designated VGAM RNA, also designated SEQ ID:491.

Another function of VGAM156 is therefore inhibition of DKFZP56400463 (Accession NM_{—}014156). Accordingly, utilities of VGAM156 include diagnosis, prevention and treatment of diseases and clinical conditions associated with DKFZP56400463. Glutamate Receptor, Ionotropic, Delta 1 (GRID1, Accession XM_{—}043613) is another VGAM156 host target gene. GRID1 BINDING SITE is HOST TARGET binding site found in the 3′ untranslated region of mRNA encoded by GRID1, corresponding to a HOST TARGET binding site such as BINDING SITE I, BINDING SITE II or BINDING SITE III. Table 2 illustrates the complementarity of the nucleotide sequences of GRID1 BINDING SITE, designated SEQ ID:2822, to the nucleotide sequence of VGAM156 RNA, herein designated VGAM RNA, also designated SEQ ID:491.

Another function of VGAM156 is therefore inhibition of Glutamate Receptor, Ionotropic, Delta 1 (GRID1, Accession XM_{—}043613). Accordingly, utilities of VGAM156 include diagnosis, prevention and treatment of diseases and clinical conditions associated with GRID1. MGC30052 (Accession NM_{—}144721) is another VGAM156 host target gene. MGC30052 BINDING SITE is HOST TARGET binding site found in the 3′ untranslated region of mRNA encoded by MGC30052, corresponding to a HOST TARGET binding site such as BINDING SITE I, BINDING SITE II or BINDING SITE III. Table 2 illustrates the complementarity of the nucleotide sequences of MGC30052 BINDING SITE, designated SEQ ID:2499, to the nucleotide sequence of VGAM156 RNA, herein designated VGAM RNA, also designated SEQ ID:491.

Another function of VGAM156 is therefore inhibition of MGC30052 (Accession NM_{—}144721). Accordingly, utilities of VGAM156 include diagnosis, prevention and treatment of diseases and clinical conditions associated with MGC30052. MGC32104 (Accession NM_{—}144684) is another VGAM156 host target gene. MGC32104 BINDING SITE is HOST TARGET binding site found in the 5′ untranslated region of mRNA encoded by MGC32104, corresponding to a HOST TARGET binding site such as BINDING SITE I, BINDING SITE II or BINDING SITE III. Table 2 illustrates the complementarity of the nucleotide sequences of MGC32104 BINDING SITE, designated SEQ ID:2494, to the nucleotide sequence of VGAM156 RNA, herein designated VGAM RNA, also designated SEQ ID:491.

Another function of VGAM156 is therefore inhibition of MGC32104 (Accession NM_{—}144684). Accordingly, utilities of VGAM156 include diagnosis, prevention and treatment of diseases and clinical conditions associated with MGC32104. Sodium Channel, Voltage-gated, Type IX, Alpha Polypeptide (SCN9A, Accession NM_{—}002977) is another VGAM156 host target gene. SCN9A BINDING SITE is HOST TARGET binding site found in the 5′ untranslated region of mRNA encoded by SCN9A, corresponding to a HOST TARGET binding site such as BINDING SITE I, BINDING SITE II or BINDING SITE III. Table 2 illustrates the complementarity of the nucleotide sequences of SCN9A BINDING SITE, designated SEQ ID:975, to the nucleotide sequence of VGAM156 RNA, herein designated VGAM RNA, also designated SEQ ID:491.

Another function of VGAM156 is therefore inhibition of Sodium Channel, Voltage-gated, Type IX, Alpha Polypeptide (SCN9A, Accession NM_{—}002977). Accordingly, utilities of VGAM156 include diagnosis, prevention and treatment of diseases and clinical conditions associated with SCN9A. Serine/threonine Kinase 17a (apoptosis-inducing) (STK17A, Accession NM_{—}004760) is another VGAM156 host target gene. STK17A BINDING SITE is HOST TARGET binding site found in the 3′ untranslated region of mRNA encoded by STK17A, corresponding to a HOST TARGET binding site such as BINDING SITE I, BINDING SITE II or BINDING SITE III. Table 2 illustrates the complementarity of the nucleotide sequences of STK17A BINDING SITE, designated SEQ ID:1154, to the nucleotide sequence of VGAM156 RNA, herein designated VGAM RNA, also designated SEQ ID:491.

Another function of VGAM156 is therefore inhibition of Serine/threonine Kinase 17a (apoptosis-inducing) (STK17A, Accession NM_{—}004760). Accordingly, utilities of VGAM156 include diagnosis, prevention and treatment of diseases and clinical conditions associated with STK17A. LOC148738 (Accession NM_{—}145277) is another VGAM156 host target gene. LOC148738 BINDING SITE is HOST TARGET binding site found in the 3′ untranslated region of mRNA encoded by LOC148738, corresponding to a HOST TARGET binding site such as BINDING SITE I, BINDING SITE II or BINDING SITE III. Table 2 illustrates the complementarity of the nucleotide sequences of LOC148738 BINDING SITE, designated SEQ ID:2519, to the nucleotide sequence of VGAM156 RNA, herein designated VGAM RNA, also designated SEQ ID:491.

Another function of VGAM156 is therefore inhibition of LOC148738 (Accession NM_{—}145277). Accordingly, utilities of VGAM156 include diagnosis, prevention and treatment of diseases and clinical conditions associated with LOC148738. FIG. 1 further provides a conceptual description of a novel bioinformatically detected viral gene of the present invention, referred to here as Viral Genomic Address Messenger 157 (VGAM157) viral gene, which modulates expression of respective host target genes thereof, the function and utility of which host target genes is known in the art.

VGAM157 is a novel bioinformatically detected regulatory, non protein coding, viral micro RNA (miRNA) gene. The method by which VGAM157 was detected is described hereinabove with reference to FIGS. 1-8.

VGAM157 gene, herein designated VGAM GENE, is a viral gene contained in the genome of Vaccinia Virus. VGAM157 host target gene, herein designated VGAM HOST TARGET GENE, is a human gene contained in the human genome.

VGAM157 gene encodes a VGAM157 precursor RNA, herein designated VGAM PRECURSOR RNA. Similar to other miRNA genes, and unlike most ordinary genes, VGAM157 precursor RNA does not encode a protein. A nucleotide sequence identical or highly similar to the nucleotide sequence of VGAM157 precursor RNA is designated SEQ ID:143, and is provided hereinbelow with reference to the sequence listing part. Nucleotide sequence SEQ ID: 143 is located at position 79119 relative to the genome of Vaccinia Virus.

VGAM157 precursor RNA folds onto itself, forming VGAM157 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM157 folded precursor RNA into VGAM157 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM157 RNA is designated SEQ ID:492, and is provided hereinbelow with reference to the sequence listing part.

VGAM157 host target gene, herein designated VGAM HOST TARGET GENE, encodes a corresponding messenger RNA, VGAM157 host target RNA, herein designated VGAM HOST TARGET RNA. VGAM157 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM157 RNA, herein designated VGAM RNA, binds complementarily to one or more host target binding sites located in untranslated regions of VGAM157 host target RNA, herein designated VGAM HOST TARGET RNA. This complementary binding is due to the fact that the nucleotide sequence of VGAM157 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM157 RNA, herein designated VGAM RNA, may have a different number of host target binding sites in untranslated regions of a VGAM157 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM157 RNA, herein designated VGAM RNA, to host target binding sites on VGAM157 host target RNA, herein designated VGAM HOST TARGET RNA, such as BINDING SITE I, BINDING SITE II and BINDING SITE III, inhibits translation of VGAM157 host target RNA into VGAM157 host target protein, herein designated VGAM HOST TARGET PROTEIN. VGAM host target protein is therefore outlined by a broken line.

It is appreciated that VGAM157 host target gene, herein designated VGAM HOST TARGET GENE, in fact represents a plurality of VGAM157 host target genes. The mRNA of each one of this plurality of VGAM157 host target genes comprises one or more host target binding sites, each having a nucleotide sequence which is at least partly complementary to VGAM157 RNA, herein designated VGAM RNA, and which when bound by VGAM157 RNA causes inhibition of translation of respective one or more VGAM157 host target proteins.

It is further appreciated by one skilled in the art that the mode of translational inhibition illustrated by FIG. 1 with specific reference to translational inhibition exerted by VGAM157 gene, herein designated VGAM GENE, on one or more VGAM157 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM157 is inhibition of expression of host target genes, as part of a novel viral mechanism of attacking a host. Accordingly, utilities of VGAM157 include diagnosis, prevention and treatment of viral infection by Vaccinia Virus. Specific functions, and accordingly utilities, of VGAM157 correlate with, and may be deduced from, the identity of the host target genes which VGAM157 binds and inhibits, and the function of these host target genes, as elaborated hereinbelow.

Nucleotide sequences of the VGAM157 precursor RNA, herein designated VGAM PRECURSOR RNA, and of the ‘diced’ VGAM157 RNA, herein designated VGAM RNA, and a schematic representation of the secondary folding of VGAM157 folded precursor RNA, herein designated VGAM FOLDED PRECURSOR RNA, of VGAM157 are further described hereinbelow with reference to Table 1.

Nucleotide sequences of host target binding sites, such as BINDING SITE-I, BINDING SITE-II and BINDING SITE-III of FIG. 1, found on VGAM157 host target RNA, and schematic representation of the complementarity of each of these host target binding sites to VGAM157 RNA, herein designated VGAM RNA, are described hereinbelow with reference to Table 2.

As mentioned hereinabove with reference to FIG. 1, a function of VGAM157 gene, herein designated VGAM is inhibition of expression of VGAM157 target genes. It is appreciated that specific functions, and accordingly utilities, of VGAM157 correlate with, and may be deduced from, the identity of the target genes which VGAM157 binds and inhibits, and the function of these target genes, as elaborated hereinbelow.

Bleomycin Hydrolase (BLMH, Accession NM_{—}000386) is a VGAM157 host target gene. BLMH BINDING SITE is HOST TARGET binding site found in the 3′ untranslated region of mRNA encoded by BLMH, corresponding to a HOST TARGET binding site such as BINDING SITE I, BINDING SITE II or BINDING SITE III. Table 2 illustrates the complementarity of the nucleotide sequences of BLMH BINDING SITE, designated SEQ ID:734, to the nucleotide sequence of VGAM157 RNA, herein designated VGAM RNA, also designated SEQ ID:492.

A function of VGAM157 is therefore inhibition of Bleomycin Hydrolase (BLMH, Accession NM_{—}000386), a gene which is a cysteine protease that inactivates bleomycin. Accordingly, utilities of VGAM157 include diagnosis, prevention and treatment of diseases and clinical conditions associated with BLMH. The function of BLMH has been established by previous studies. Using PCR-SSCP, Bromme et al. (1996) and Montoya et al. (1997) found a 1450A-G polymorphism of the BMH gene that resulted in an ile443-to-val (1443V) conserved amino acid substitution in the carboxy terminus of the protein (602403.0001). BMH is suspected of being the unknown beta secretase that cleaves off the amyloid beta fragment from the amyloid precursor protein (APP; 104760) associated with Alzheimer disease (AD). In pooled AD cases and controls, the frequency of the 1450A and 1450G alleles were statistically indistinguishable. The G/G homozygote genotype distribution, however, was significantly different between AD cases (12.7%) and controls (6.6%); (P less than 0.001). Significant differences were not seen in the A/A homozygote and A/G heterozygote genotype distributions. Further studies by Montoya et al. (1998) indicated that the frequency of the G/G homozygote was significantly higher in AD cases than in controls only in the non-APOE4 (see OMIM Ref. No. 107741) group (15.9% in cases vs 4.7% in controls). The odds ratio for developing AD with BMH G/G genotype in the absence of an APOE4 allele was 3.81; in the presence of an APOE4 allele, the odds ratio was 0.98. Farrer et al. (1998) were unable to confirm this association in a sample of 621 Caucasian Alzheimer patients drawn from 4 North American university-based research centers. Susceptibility to bleomycin-induced chromatid breaks in cultured peripheral blood lymphocytes may reflect the way a person deals with carcinogenic challenges (Hsu et al., 1989). This susceptibility, also referred to as mutagen sensitivity, has been found to be increased in patients with environmentally related cancers, including cancers of the head and neck, lung, and colon. In combination with carcinogenic exposure, this susceptibility can greatly influence cancer risk. Cloos et al. (1999) determined the number of bleomycin-induced breaks per cell for 135 healthy volunteers without cancer. These individuals were from 53 different pedigrees and included 25 monozygotic twin pairs, 14 pairs of dizygotics, and 14 families selected on the basis of a first-degree relative who was successfully treated for head and neck cancer and who had no sign of recurrence for at least 1 year. Results showed no evidence for the influence of a shared family environment on bleomycin-induced chromatid breaks. On the other hand, genetic influences were statistically significant and accounted for 75% of the total variance.

Full details of the abovementioned studies are described in the following publications, the disclosure of which are hereby incorporated by reference:
Bromme, D.; Rossi, A. B.; Smeekens, S. P.; Anderson, D. C.; Payan, D. G.: Human bleomycin hydrolase: molecular cloning, sequencing, functional expression, and enzymatic characterization. Biochemistry 35: 6706-6714, 1996.; and
Hsu, T. C.; Johnston, D. A.; Cherry, L. M.; Ramkissoon, D.; Schantz, S. P.; Jessup, J. M.; Winn, R. J.; Shirley, L.; Furlong, C.: Sensitivity to genotoxic effects of bleomycin in human.

Further studies establishing the function and utilities of BLMH are found in John Hopkins OMIM database record ID 602403, and in sited publications numbered 1461-147 and 1470-1471 listed in the bibliography section hereinbelow, which are also hereby incorporated by reference. Biliverdin Reductase B (flavin reductase (NADPH)) (BLVRB, Accession NM_{—}000713) is another VGAM157 host target gene. BLVRB BINDING SITE is HOST TARGET binding site found in the 3′ untranslated region of mRNA encoded by BLVRB, corresponding to a HOST TARGET binding site such as BINDING SITE I, BINDING SITE II or BINDING SITE III. Table 2 illustrates the complementarity of the nucleotide sequences of BLVRB BINDING SITE, designated SEQ ID:768, to the nucleotide sequence of VGAM157 RNA, herein designated VGAM RNA, also designated SEQ ID:492.

Another function of VGAM157 is therefore inhibition of Biliverdin Reductase B (flavin reductase (NADPH)) (BLVRB, Accession NM_{—}000713), a gene which involves in protecting cells from oxidative damage or in regulating iron metabolism. Accordingly, utilities of VGAM157 include diagnosis, prevention and treatment of diseases and clinical conditions associated with BLVRB. The function of BLVRB has been established by previous studies. The final step in heme metabolism in mammals is catalyzed by the cytosolic enzyme biliverdin reductase (EC 1.3.1.24). Yamaguchi et al. (1993) isolated 2 biliverdin reductases. BLVRA (OMIM Ref. No. 109750), which they called biliverdin-IX alpha-reductase, is a major component of human adult liver and is identical to the enzyme previously reported as biliverdin reductase. BLVRB, which they called biliverdin-IX beta-reductase, is found predominantly in fetal liver. NADPH-flavin reductase (FLR) of erythrocytes has been studied as one of the methemoglobin-reducing enzymes. Chikuba et al. (1994) isolated a cDNA from a human reticulocyte library that encodes a predicted 206-amino acid FLR. Western blots of rat tissues showed highest levels of FLR in the erythrocytes and liver. Northern blots indicated a 1.1-kb transcript in human tissues.

Full details of the abovementioned studies are described in the following publications, the disclosure of which are hereby incorporated by reference:
Yamaguchi, T.; Komuro, A.; Nakano, Y.; Tomita, M.; Nakajima, H.: Complete amino acid sequence of biliverdin-IX beta reductase from human liver. Biochem. Biophys. Res. Commun. 197: 1518-1523, 1993.; and
Chikuba, K.; Yubisui, T.; Shirabe, K.; Takeshita, M.: Cloning and nucleotide sequence of a cDNA of the human erythrocyte NADPH-flavin reductase. Biochem. Biophys. Res. Commun. 198: 1170.

Further studies establishing the function and utilities of BLVRB are found in John Hopkins OMIM database record ID 600941, and in sited publications numbered 317-319 listed in the bibliography section hereinbelow, which are also hereby incorporated by reference. 7-dehydrocholesterol Reductase (DHCR7, Accession NM_{—}001360) is another VGAM157 host target gene. DHCR7 BINDING SITE is HOST TARGET binding site found in the 3′ untranslated region of mRNA encoded by DHCR7, corresponding to a HOST TARGET binding site such as BINDING SITE I, BINDING SITE II or BINDING SITE III. Table 2 illustrates the complementarity of the nucleotide sequences of DHCR7 BINDING SITE, designated SEQ ID:821, to the nucleotide sequence of VGAM157 RNA, herein designated VGAM RNA, also designated SEQ ID:492.

Another function of VGAM157 is therefore inhibition of 7-dehydrocholesterol Reductase (DHCR7, Accession NM_{—}001360). Accordingly, utilities of VGAM157 include diagnosis, prevention and treatment of diseases and clinical conditions associated with DHCR7. Fibroblast Growth Factor Receptor 1 (fms-related tyrosine kinase 2, Pfeiffer syndrome) (FGFR1, Accession NM_{—}023107) is another VGAM157 host target gene. FGFR1 BINDING SITE1 and FGFR1 BINDING SITE2 are HOST TARGET binding sites found in untranslated regions of mRNA encoded by FGFR1, corresponding to HOST TARGET binding sites such as BINDING SITE I, BINDING SITE II or BINDING SITE III. Table 2 illustrates the complementarity of the nucleotide sequences of FGFR1 BINDING SITE1 and FGFR1 BINDING SITE2, designated SEQ ID:2032 and SEQ ID:2034 respectively, to the nucleotide sequence of VGAM157 RNA, herein designated VGAM RNA, also designated SEQ ID:492.

Another function of VGAM157 is therefore inhibition of Fibroblast Growth Factor Receptor 1 (fms-related tyrosine kinase 2, Pfeiffer syndrome) (FGFR1, Accession NM_{—}023107). Accordingly, utilities of VGAM157 include diagnosis, prevention and treatment of diseases and clinical conditions associated with FGFR1. 5′-nucleotidase, Cytosolic III (NT5C3, Accession NM_{—}016489) is another VGAM157 host target gene. NT5C3 BINDING SITE is HOST TARGET binding site found in the 5′ untranslated region of mRNA encoded by NT5C3, corresponding to a HOST TARGET binding site such as BINDING SITE I, BINDING SITE II or BINDING SITE III. Table 2 illustrates the complementarity of the nucleotide sequences of NT5C3 BINDING SITE, designated SEQ ID: 1689, to the nucleotide sequence of VGAM157 RNA, herein designated VGAM RNA, also designated SEQ ID:492.

Another function of VGAM157 is therefore inhibition of 5′-nucleotidase, Cytosolic III (NT5C3, Accession NM_{—}016489). Accordingly, utilities of VGAM157 include diagnosis, prevention and treatment of diseases and clinical conditions associated with NT5C3. Otoraplin (OTOR, Accession NM_{—}020157) is another VGAM157 host target gene. OTOR BINDING SITE is HOST TARGET binding site found in the 3′ untranslated region of mRNA encoded by OTOR, corresponding to a HOST TARGET binding site such as BINDING SITE I, BINDING SITE II or BINDING SITE III. Table 2 illustrates the complementarity of the nucleotide sequences of OTOR BINDING SITE, designated SEQ ID: 1894, to the nucleotide sequence of VGAM157 RNA, herein designated VGAM RNA, also designated SEQ ID:492.

Another function of VGAM157 is therefore inhibition of Otoraplin (OTOR, Accession NM_{—}020157), a gene which plays a role in the early chondrogenesis of the periotic mesenchyme. Accordingly, utilities of VGAM157 include diagnosis, prevention and treatment of diseases and clinical conditions associated with OTOR. The function of OTOR has been established by previous studies. During large-scale analysis of human fetal cochlea EST sequences, Robertson et al. (2000) identified a novel gene, designated OTOR. They also isolated orthologous genes in mouse, chicken, and bullfrog by screening inner ear cDNA libraries. Rendtorff et al. (2001) independently identified OTOR, which they called MIA-like (MIAL), during a search for cochlea-specific EST clusters. Cohen-Salmon et al. (2000) cloned the mouse Otor gene, which they called fibrocyte-derived protein (Fdp), using a subtracted mouse cochlea cDNA library and 5-prime RACE. Human OTOR encodes a 128-amino acid protein with a predicted secretion signal peptide. The protein shares high sequence identity with the mouse (90%), chicken (80%), and bullfrog (60%) orthologs and with the related human CDRAP/MIA protein (OMIM Ref. No. 601340) (43%). By expressing OTOR in mammalian cell cultures, Rendtorff et al. (2001) showed that OTOR is translated as an approximately 15-kD polypeptide that is assembled into a covalently linked homodimer, modified by sulfation, and secreted from the cells via the Golgi apparatus. By Northern blot analysis, Robertson et al. (2000) detected expression of a major 1.1-kb and minor 1.8- and 4-kb OTOR transcripts in human cochlea. Using RT-PCR and in situ hybridization, Rendtorff et al. (2001) detected OTOR expression specific to a cell layer beneath the sensory epithelium of cochlea and vestibule of human fetal inner ear. Rendtorff et al. (2001) reported a frequent polymorphism in the translation initiation codon of OTOR (ACG instead of ATG). Of 505 unrelated individuals analyzed, 9.5% were ACG/ATG heterozygous. The ACG allele failed to direct synthesis of the OTOR protein in transfected cells. Rendtorff et al. (2001) concluded that OTOR may contribute to inner ear dysfunction in humans.

Full details of the abovementioned studies are described in the following publications, the disclosure of which are hereby incorporated by reference:
Rendtorff, N. D.; Frodin, M.; Attie-Bitach, T.; Vekemans, M.; Tommerup, N.: Identification and characterization of an inner ear-expressed human melanoma inhibitory activity (MIA)-like gene (MIAL) with a frequent polymorphism that abolishes translation. Genomics 71: 40-52, 2001.; and
Robertson, N. G.; Heller, S.; Lin, J. S.; Resendes, B. L.; Weremowicz, S.; Denis, C. S.; Bell, A. M.; Hudspeth, A. J.; Morton, C. C.: A novel conserved cochlear gene, OTOR: identification.

Further studies establishing the function and utilities of OTOR are found in John Hopkins OMIM database record ID 606067, and in sited publications numbered 1004-1006 listed in the bibliography section hereinbelow, which are also hereby incorporated by reference. Phosphoribosyl Pyrophosphate Synthetase-associated Protein 2 (PRPSAP2, Accession NM_{—}002767) is another VGAM157 host target gene. PRPSAP2 BINDING SITE is HOST TARGET binding site found in the 5′ untranslated region of mRNA encoded by PRPSAP2, corresponding to a HOST TARGET binding site such as BINDING SITE I, BINDING SITE II or BINDING SITE III. Table 2 illustrates the complementarity of the nucleotide sequences of PRPSAP2 BINDING SITE, designated SEQ ID:954, to the nucleotide sequence of VGAM157 RNA, herein designated VGAM RNA, also designated SEQ ID:492.

Another function of VGAM157 is therefore inhibition of Phosphoribosyl Pyrophosphate Synthetase-associated Protein 2 (PRPSAP2, Accession NM_{—}002767), a gene which may be a negative regulator of phosphoribosyl pyrophosphate synthesis. Accordingly, utilities of VGAM157 include diagnosis, prevention and treatment of diseases and clinical conditions associated with PRPSAP2. The function of PRPSAP2 has been established by previous studies. Phosphoribosylpyrophosphate (PRPP) is a primary substrate and a critical control factor for de novo synthesis of purine and pyrimidine nucleotides, histidine, tryptophan, and NAD. The enzyme PRPP synthetase (PRS), which catalyzes the formation of PRPP, exists in an aggregate composed of 2 catalytic subunits, PRSI (OMIM Ref. No. 311850) and PRSII (OMIM Ref. No. 311860), and 2 associated subunits, PAP39 (OMIM Ref. No. 601249) and PAP41. By searching an EST database for sequences related to PAP39, Katashima et al. (1998) identified cDNAs encoding PAP41. The predicted 369-amino acid protein exhibited 72%, 51%, and 50% sequence identity with PAP39, PRSI, and PRSII, respectively. The rat and human PAP41 proteins are 99% identical. Northern blot analysis revealed that PAP41 is expressed as a 2.1-kb mRNA in several human cell lines. By fluorescence in situ hybridization, Katashima et al. (1998) mapped the PRPSAP2 gene to 17p12-p11.2.

Full details of the abovementioned studies are described in the following publications, the disclosure of which are hereby incorporated by reference:
Katashima, R.; Iwahana, H.; Fujimura, M.; Yamaoka, T.; Ishizuka, T.; Tatibana, M.; Itakura, M.: Molecular cloning of a human cDNA for the 41-kDa phosphoribosylpyrophosphate synthetase-associated protein. Biochim. Biophys. Acta 1396: 245-250, 1998.; and
Katashima, R.; Iwahana, H.; Fujimura, M.; Yamaoka, T.; Itakura, M.: Assignment of the human phosphoribosylpyrophosphate synthetase-associated protein 41 gene (PRPSAP2) to 17p11.2-p12. G.

Further studies establishing the function and utilities of PRPSAP2 are found in John Hopkins OMIM database record ID 603762, and in sited publications numbered 1746-1747 listed in the bibliography section hereinbelow, which are also hereby incorporated by reference. Chromosome 14 Open Reading Frame 4 (C14orf4, Accession XM_{—}041104) is another VGAM157 host target gene. C14orf4 BINDING SITE is HOST TARGET binding site found in the 5′ untranslated region of mRNA encoded by C14orf4, corresponding to a HOST TARGET binding site such as BINDING SITE I, BINDING SITE II or BINDING SITE III. Table 2 illustrates the complementarity of the nucleotide sequences of C14orf4 BINDING SITE, designated SEQ ID:2788, to the nucleotide sequence of VGAM157 RNA, herein designated VGAM RNA, also designated SEQ ID:492.

Another function of VGAM157 is therefore inhibition of Chromosome 14 Open Reading Frame 4 (C14orf4, Accession XM_{—}041104). Accordingly, utilities of VGAM157 include diagnosis, prevention and treatment of diseases and clinical conditions associated with C14orf4. CG012 (Accession XM_{—}096710) is another VGAM157 host target gene. CG012 BINDING SITE is HOST TARGET binding site found in the 3′ untranslated region of mRNA encoded by CG012, corresponding to a HOST TARGET binding site such as BINDING SITE I, BINDING SITE II or BINDING SITE III. Table 2 illustrates the complementarity of the nucleotide sequences of CG012 BINDING SITE, designated SEQ ID:3272, to the nucleotide sequence of VGAM157 RNA, herein designated VGAM RNA, also designated SEQ ID:492.

Another function of VGAM157 is therefore inhibition of CG012 (Accession XM_{—}096710). Accordingly, utilities of VGAM157 include diagnosis, prevention and treatment of diseases and clinical conditions associated with CG012. DKFZP434K2235 (Accession XM_{—}096869) is another VGAM157 host target gene. DKFZP434K2235 BINDING SITE is HOST TARGET binding site found in the 5′ untranslated region of mRNA encoded by DKFZP434K2235, corresponding to a HOST TARGET binding site such as BINDING SITE I, BINDING SITE II or BINDING SITE III. Table 2 illustrates the complementarity of the nucleotide sequences of DKFZP434K2235 BINDING SITE, designated SEQ ID:3278, to the nucleotide sequence of VGAM157 RNA, herein designated VGAM RNA, also designated SEQ ID:492.

Another function of VGAM157 is therefore inhibition of DKFZP434K2235 (Accession XM_{—}096869). Accordingly, utilities of VGAM157 include diagnosis, prevention and treatment of diseases and clinical conditions associated with DKFZP434K2235. FLJ20699 (Accession NM_{—}017931) is another VGAM157 host target gene. FLJ20699 BINDING SITE is HOST TARGET binding site found in the 3′ untranslated region of mRNA encoded by FLJ20699, corresponding to a HOST TARGET binding site such as BINDING SITE I, BINDING SITE II or BINDING SITE III. Table 2 illustrates the complementarity of the nucleotide sequences of FLJ20699 BINDING SITE, designated SEQ ID:1766, to the nucleotide sequence of VGAM157 RNA, herein designated VGAM RNA, also designated SEQ ID:492.

Another function of VGAM157 is therefore inhibition of FLJ20699 (Accession NM_{—}017931). Accordingly, utilities of VGAM157 include diagnosis, prevention and treatment of diseases and clinical conditions associated with FLJ20699. FLJ23129 (Accession NM_{—}024763) is another VGAM157 host target gene. FLJ23129 BINDING SITE is HOST TARGET binding site found in the 3′ untranslated region of mRNA encoded by FLJ23129, corresponding to a HOST TARGET binding site such as BINDING SITE I, BINDING SITE II or BINDING SITE III. Table 2 illustrates the complementarity of the nucleotide sequences of FLJ23129 BINDING SITE, designated SEQ ID:2088, to the nucleotide sequence of VGAM157 RNA, herein designated VGAM RNA, also designated SEQ ID:492.

Another function of VGAM157 is therefore inhibition of FLJ23129 (Accession NM_{—}024763). Accordingly, utilities of VGAM157 include diagnosis, prevention and treatment of diseases and clinical conditions associated with FLJ23129. HSPC065 (Accession NM_{—}014157) is another VGAM157 host target gene. HSPC065 BINDING SITE is HOST TARGET binding site found in the 3′ untranslated region of mRNA encoded by HSPC065, corresponding to a HOST TARGET binding site such as BINDING SITE I, BINDING SITE II or BINDING SITE III. Table 2 illustrates the complementarity of the nucleotide sequences of HSPC065 BINDING SITE, designated SEQ ID: 1482, to the nucleotide sequence of VGAM157 RNA, herein designated VGAM RNA, also designated SEQ ID:492.

Another function of VGAM157 is therefore inhibition of HSPC065 (Accession NM_{—}014157). Accordingly, utilities of VGAM157 include diagnosis, prevention and treatment of diseases and clinical conditions associated with HSPC065. KIAA0960 (Accession XM_{—}166543) is another VGAM157 host target gene. KIAA0960 BINDING SITE is HOST TARGET binding site found in the 3′ untranslated region of mRNA encoded by KIAA0960, corresponding to a HOST TARGET binding site such as BINDING SITE I, BINDING SITE II or BINDING SITE III. Table 2 illustrates the complementarity of the nucleotide sequences of KIAA0960 BINDING SITE, designated SEQ ID:3572, to the nucleotide sequence of VGAM157 RNA, herein designated VGAM RNA, also designated SEQ ID:492.

Another function of VGAM157 is therefore inhibition of KIAA0960 (Accession XM_{—}166543). Accordingly, utilities of VGAM157 include diagnosis, prevention and treatment of diseases and clinical conditions associated with KIAA0960. MGC12466 (Accession XM_{—}086336) is another VGAM157 host target gene. MGC12466 BINDING SITE is HOST TARGET binding site found in the 3′ untranslated region of mRNA encoded by MGC12466, corresponding to a HOST TARGET binding site such as BINDING SITE I, BINDING SITE II or BINDING SITE III. Table 2 illustrates the complementarity of the nucleotide sequences of MGC12466 BINDING SITE, designated SEQ ID:3127, to the nucleotide sequence of VGAM157 RNA, herein designated VGAM RNA, also designated SEQ ID:492.

Another function of VGAM157 is therefore inhibition of MGC12466 (Accession XM_{—}086336). Accordingly, utilities of VGAM157 include diagnosis, prevention and treatment of diseases and clinical conditions associated with MGC12466. MGC12904 (Accession NM_{—}031219) is another VGAM157 host target gene. MGC12904 BINDING SITE is HOST TARGET binding site found in the 3′ untranslated region of mRNA encoded by MGC12904, corresponding to a HOST TARGET binding site such as BINDING SITE I, BINDING SITE II or BINDING SITE III. Table 2 illustrates the complementarity of the nucleotide sequences of MGC12904 BINDING SITE, designated SEQ ID:2184, to the nucleotide sequence of VGAM157 RNA, herein designated VGAM RNA, also designated SEQ ID:492.

Another function of VGAM157 is therefore inhibition of MGC12904 (Accession NM_{—}031219). Accordingly, utilities of VGAM157 include diagnosis, prevention and treatment of diseases and clinical conditions associated with MGC12904. MSTP028 (Accession NM_{—}031954) is another VGAM157 host target gene. MSTP028 BINDING SITE is HOST TARGET binding site found in the 3′ untranslated region of mRNA encoded by MSTP028, corresponding to a HOST TARGET binding site such as BINDING SITE I, BINDING SITE II or BINDING SITE III. Table 2 illustrates the complementarity of the nucleotide sequences of MSTP028 BINDING SITE, designated SEQ ID:2224, to the nucleotide sequence of VGAM157 RNA, herein designated VGAM RNA, also designated SEQ ID:492.

Another function of VGAM157 is therefore inhibition of MSTP028 (Accession NM_{—}031954). Accordingly, utilities of VGAM157 include diagnosis, prevention and treatment of diseases and clinical conditions associated with MSTP028. Reserved (RAB24, Accession NM_{—}130781) is another VGAM157 host target gene. RAB24 BINDING SITE is HOST TARGET binding site found in the 5′ untranslated region of mRNA encoded by RAB24, corresponding to a HOST TARGET binding site such as BINDING SITE I, BINDING SITE II or BINDING SITE III. Table 2 illustrates the complementarity of the nucleotide sequences of RAB24 BINDING SITE, designated SEQ ID:2397, to the nucleotide sequence of VGAM157 RNA, herein designated VGAM RNA, also designated SEQ ID:492.

Another function of VGAM157 is therefore inhibition of Reserved (RAB24, Accession NM_{—}130781). Accordingly, utilities of VGAM157 include diagnosis, prevention and treatment of diseases and clinical conditions associated with RAB24. LOC113828 (Accession NM_{—}138435) is another VGAM157 host target gene. LOC113828 BINDING SITE is HOST TARGET binding site found in the 5′ untranslated region of mRNA encoded by LOC113828, corresponding to a HOST TARGET binding site such as BINDING SITE I, BINDING SITE II or BINDING SITE III. Table 2 illustrates the complementarity of the nucleotide sequences of LOC113828 BINDING SITE, designated SEQ ID:2439, to the nucleotide sequence of VGAM157 RNA, herein designated VGAM RNA, also designated SEQ ID:492.

Another function of VGAM157 is therefore inhibition of LOC113828 (Accession NM_{—}138435). Accordingly, utilities of VGAM157 include diagnosis, prevention and treatment of diseases and clinical conditions associated with LOC113828. LOC152300 (Accession XM_{—}087432) is another VGAM157 host target gene. LOC152300 BINDING SITE is HOST TARGET binding site found in the 5′ untranslated region of mRNA encoded by LOC152300, corresponding to a HOST TARGET binding site such as BINDING SITE I, BINDING SITE II or BINDING SITE III. Table 2 illustrates the complementarity of the nucleotide sequences of LOC152300 BINDING SITE, designated SEQ ID:3169, to the nucleotide sequence of VGAM157 RNA, herein designated VGAM RNA, also designated SEQ ID:492.

Another function of VGAM157 is therefore inhibition of LOC152300 (Accession XM_{—}087432). Accordingly, utilities of VGAM157 include diagnosis, prevention and treatment of diseases and clinical conditions associated with LOC152300. LOC197125 (Accession XM_{—}113826) is another VGAM157 host target gene. LOC197125 BINDING SITE is HOST TARGET binding site found in the 3′ untranslated region of mRNA encoded by LOC197125, corresponding to a HOST TARGET binding site such as BINDING SITE I, BINDING SITE II or BINDING SITE III. Table 2 illustrates the complementarity of the nucleotide sequences of LOC197125 BINDING SITE, designated SEQ ID:3420, to the nucleotide sequence of VGAM157 RNA, herein designated VGAM RNA, also designated SEQ ID:492.

Another function of VGAM157 is therefore inhibition of LOC197125 (Accession XM_{—}113826). Accordingly, utilities of VGAM157 include diagnosis, prevention and treatment of diseases and clinical conditions associated with LOC197125. LOC219848 (Accession XM_{—}166170) is another VGAM157 host target gene. LOC219848 BINDING SITE is HOST TARGET binding site found in the 3′ untranslated region of mRNA encoded by LOC219848, corresponding to a HOST TARGET binding site such as BINDING SITE I, BINDING SITE II or BINDING SITE III. Table 2 illustrates the complementarity of the nucleotide sequences of LOC219848 BINDING SITE, designated SEQ ID:3528, to the nucleotide sequence of VGAM157 RNA, herein designated VGAM RNA, also designated SEQ ID:492.

Another function of VGAM157 is therefore inhibition of LOC219848 (Accession XM_{—}166170). Accordingly, utilities of VGAM157 include diagnosis, prevention and treatment of diseases and clinical conditions associated with LOC219848. LOC51157 (Accession NM_{—}016202) is another VGAM157 host target gene. LOC51157 BINDING SITE is HOST TARGET binding site found in the 5′ untranslated region of mRNA encoded by LOC51157, corresponding to a HOST TARGET binding site such as BINDING SITE I, BINDING SITE II or BINDING SITE III. Table 2 illustrates the complementarity of the nucleotide sequences of LOC51157 BINDING SITE, designated SEQ ID:1671, to the nucleotide sequence of VGAM157 RNA, herein designated VGAM RNA, also designated SEQ ID:492.

Another function of VGAM157 is therefore inhibition of LOC51157 (Accession NM_{—}016202). Accordingly, utilities of VGAM157 include diagnosis, prevention and treatment of diseases and clinical conditions associated with LOC51157. LOC92270 (Accession XM_{—}043989) is another VGAM157 host target gene. LOC92270 BINDING SITE is HOST TARGET binding site found in the 5′ untranslated region of mRNA encoded by LOC92270, corresponding to a HOST TARGET binding site such as BINDING SITE I, BINDING SITE II or BINDING SITE III. Table 2 illustrates the complementarity of the nucleotide sequences of LOC92270 BINDING SITE, designated SEQ ID:2828, to the nucleotide sequence of VGAM157 RNA, herein designated VGAM RNA, also designated SEQ ID:492.

Another function of VGAM157 is therefore inhibition of LOC92270 (Accession XM_{—}043989). Accordingly, utilities of VGAM157 include diagnosis, prevention and treatment of diseases and clinical conditions associated with LOC92270. FIG. 1 further provides a conceptual description of a novel bioinformatically detected viral gene of the present invention, referred to here as Viral Genomic Address Messenger 158 (VGAM158) viral gene, which modulates expression of respective host target genes thereof, the function and utility of which host target genes is known in the art.

VGAM158 is a novel bioinformatically detected regulatory, non protein coding, viral micro RNA (miRNA) gene. The method by which VGAM158 was detected is described hereinabove with reference to FIGS. 1-8.

VGAM158 gene, herein designated VGAM GENE, is a viral gene contained in the genome of Vaccinia Virus. VGAM158 host target gene, herein designated VGAM HOST TARGET GENE, is a human gene contained in the human genome.

VGAM158 gene encodes a VGAM158 precursor RNA, herein designated VGAM PRECURSOR RNA. Similar to other miRNA genes, and unlike most ordinary genes, VGAM158 precursor RNA does not encode a protein. A nucleotide sequence identical or highly similar to the nucleotide sequence of VGAM158 precursor RNA is designated SEQ ID:144, and is provided hereinbelow with reference to the sequence listing part. Nucleotide sequence SEQ ID: 144 is located at position 81571 relative to the genome of Vaccinia Virus.

VGAM158 precursor RNA folds onto itself, forming VGAM158 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM158 folded precursor RNA into VGAM158 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM158 RNA is designated SEQ ID:493, and is provided hereinbelow with reference to the sequence listing part.

VGAM158 host target gene, herein designated VGAM HOST TARGET GENE, encodes a corresponding messenger RNA, VGAM158 host target RNA, herein designated VGAM HOST TARGET RNA. VGAM158 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM158 RNA, herein designated VGAM RNA, binds complementarily to one or more host target binding sites located in untranslated regions of VGAM158 host target RNA, herein designated VGAM HOST TARGET RNA. This complementary binding is due to the fact that the nucleotide sequence of VGAM158 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM158 RNA, herein designated VGAM RNA, may have a different number of host target binding sites in untranslated regions of a VGAM158 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM158 RNA, herein designated VGAM RNA, to host target binding sites on VGAM158 host target RNA, herein designated VGAM HOST TARGET RNA, such as BINDING SITE I, BINDING SITE II and BINDING SITE III, inhibits translation of VGAM158 host target RNA into VGAM158 host target protein, herein designated VGAM HOST TARGET PROTEIN. VGAM host target protein is therefore outlined by a broken line.

It is appreciated that VGAM158 host target gene, herein designated VGAM HOST TARGET GENE, in fact represents a plurality of VGAM158 host target genes. The mRNA of each one of this plurality of VGAM158 host target genes comprises one or more host target binding sites, each having a nucleotide sequence which is at least partly complementary to VGAM158 RNA, herein designated VGAM RNA, and which when bound by VGAM158 RNA causes inhibition of translation of respective one or more VGAM158 host target proteins.

It is further appreciated by one skilled in the art that the mode of translational inhibition illustrated by FIG. 1 with specific reference to translational inhibition exerted by VGAM158 gene, herein designated VGAM GENE, on one or more VGAM158 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM158 is inhibition of expression of host target genes, as part of a novel viral mechanism of attacking a host. Accordingly, utilities of VGAM158 include diagnosis, prevention and treatment of viral infection by Vaccinia Virus. Specific functions, and accordingly utilities, of VGAM158 correlate with, and may be deduced from, the identity of the host target genes which VGAM158 binds and inhibits, and the function of these host target genes, as elaborated hereinbelow.

Nucleotide sequences of the VGAM158 precursor RNA, herein designated VGAM PRECURSOR RNA, and of the ‘diced’ VGAM158 RNA, herein designated VGAM RNA, and a schematic representation of the secondary folding of VGAM158 folded precursor RNA, herein designated VGAM FOLDED PRECURSOR RNA, of VGAM158 are further described hereinbelow with reference to Table 1.

Nucleotide sequences of host target binding sites, such as BINDING SITE-I, BINDING SITE-II and BINDING SITE-III of FIG. 1, found on VGAM158 host target RNA, and schematic representation of the complementarity of each of these host target binding sites to VGAM158 RNA, herein designated VGAM RNA, are described hereinbelow with reference to Table 2.

As mentioned hereinabove with reference to FIG. 1, a function of VGAM158 gene, herein designated VGAM is inhibition of expression of VGAM158 target genes. It is appreciated that specific functions, and accordingly utilities, of VGAM158 correlate with, and may be deduced from, the identity of the target genes which VGAM158 binds and inhibits, and the function of these target genes, as elaborated hereinbelow.

UDP-Gal: betaGlcNAc Beta 1,3-galactosyltransferase, Polypeptide 3 (B3GALT3, Accession NM_{—}003781) is a VGAM158 host target gene. B3GALT3 BINDING SITE is HOST TARGET binding site found in the 3′ untranslated region of mRNA encoded by B3GALT3, corresponding to a HOST TARGET binding site such as BINDING SITE I, BINDING SITE II or BINDING SITE III. Table 2 illustrates the complementarity of the nucleotide sequences of B3GALT3 BINDING SITE, designated SEQ ID: 1057, to the nucleotide sequence of VGAM158 RNA, herein designated VGAM RNA, also designated SEQ ID:493.

A function of VGAM158 is therefore inhibition of UDP-Gal:betaGlcNAc Beta 1,3-galactosyltransferase, Polypeptide 3 (B3GALT3, Accession NM_{—}003781). Accordingly, utilities of VGAM158 include diagnosis, prevention and treatment of diseases and clinical conditions associated with B3GALT3. KIAA1985 (Accession NM_{—}024577) is another VGAM158 host target gene. KIAA1985 BINDING SITE is HOST TARGET binding site found in the 5′ untranslated region of mRNA encoded by KIAA1985, corresponding to a HOST TARGET binding site such as BINDING SITE I, BINDING SITE II or BINDING SITE III. Table 2 illustrates the complementarity of the nucleotide sequences of KIAA1985 BINDING SITE, designated SEQ ID:2066, to the nucleotide sequence of VGAM158 RNA, herein designated VGAM RNA, also designated SEQ ID:493.

Another function of VGAM158 is therefore inhibition of KIAA1985 (Accession NM_{—}024577). Accordingly, utilities of VGAM158 include diagnosis, prevention and treatment of diseases and clinical conditions associated with KIAA1985. MCM10 Minichromosome Maintenance Deficient 10 (S. cerevisiae) (MCM10, Accession NM_{—}018518) is another VGAM158 host target gene. MCM10 BINDING SITE is HOST TARGET binding site found in the 3′ untranslated region of mRNA encoded by MCM10, corresponding to a HOST TARGET binding site such as BINDING SITE I, BINDING SITE II or BINDING SITE III. Table 2 illustrates the complementarity of the nucleotide sequences of MCM10 BINDING SITE, designated SEQ ID:1835, to the nucleotide sequence of VGAM158 RNA, herein designated VGAM RNA, also designated SEQ ID:493.

Another function of VGAM158 is therefore inhibition of MCM10 Minichromosome Maintenance Deficient 10 (S. cerevisiae) (MCM10, Accession NM_{—}018518). Accordingly, utilities of VGAM158 include diagnosis, prevention and treatment of diseases and clinical conditions associated with MCM10. LOC149707 (Accession XM_{—}086641) is another VGAM158 host target gene. LOC149707 BINDING SITE is HOST TARGET binding site found in the 5′ untranslated region of mRNA encoded by LOC149707, corresponding to a HOST TARGET binding site such as BINDING SITE I, BINDING SITE II or BINDING SITE III. Table 2 illustrates the complementarity of the nucleotide sequences of LOC149707 BINDING SITE, designated SEQ ID:3137, to the nucleotide sequence of VGAM158 RNA, herein designated VGAM RNA, also designated SEQ ID:493.

Another function of VGAM158 is therefore inhibition of LOC149707 (Accession XM_{—}086641). Accordingly, utilities of VGAM158 include diagnosis, prevention and treatment of diseases and clinical conditions associated with LOC149707. LOC200227 (Accession XM_{—}114162) is another VGAM158 host target gene. LOC200227 BINDING SITE is HOST TARGET binding site found in the 5′ untranslated region of mRNA encoded by LOC200227, corresponding to a HOST TARGET binding site such as BINDING SITE I, BINDING SITE II or BINDING SITE III. Table 2 illustrates the complementarity of the nucleotide sequences of LOC200227 BINDING SITE, designated SEQ ID:3446, to the nucleotide sequence of VGAM158 RNA, herein designated VGAM RNA, also designated SEQ ID:493.

Another function of VGAM158 is therefore inhibition of LOC200227 (Accession XM_{—}114162). Accordingly, utilities of VGAM158 include diagnosis, prevention and treatment of diseases and clinical conditions associated with LOC200227. LOC255718 (Accession XM_{—}174148) is another VGAM158 host target gene. LOC255718 BINDING SITE is HOST TARGET binding site found in the 5′ untranslated region of mRNA encoded by LOC255718, corresponding to a HOST TARGET binding site such as BINDING SITE I, BINDING SITE II or BINDING SITE III. Table 2 illustrates the complementarity of the nucleotide sequences of LOC255718 BINDING SITE, designated SEQ ID:3741, to the nucleotide sequence of VGAM158 RNA, herein designated VGAM RNA, also designated SEQ ID:493.

Another function of VGAM158 is therefore inhibition of LOC255718 (Accession XM_{—}174148). Accordingly, utilities of VGAM158 include diagnosis, prevention and treatment of diseases and clinical conditions associated with LOC255718. FIG. 1 further provides a conceptual description of a novel bioinformatically detected viral gene of the present invention, referred to here as Viral Genomic Address Messenger 159 (VGAM159) viral gene, which modulates expression of respective host target genes thereof, the function and utility of which host target genes is known in the art.

VGAM159 is a novel bioinformatically detected regulatory, non protein coding, viral micro RNA (miRNA) gene. The method by which VGAM159 was detected is described hereinabove with reference to FIGS. 1-8.

VGAM159 gene, herein designated VGAM GENE, is a viral gene contained in the genome of Vaccinia Virus. VGAM159 host target gene, herein designated VGAM HOST TARGET GENE, is a human gene contained in the human genome.

VGAM159 gene encodes a VGAM159 precursor RNA, herein designated VGAM PRECURSOR RNA. Similar to other miRNA genes, and unlike most ordinary genes, VGAM159 precursor RNA does not encode a protein. A nucleotide sequence identical or highly similar to the nucleotide sequence of VGAM159 precursor RNA is designated SEQ ID:145, and is provided hereinbelow with reference to the sequence listing part. Nucleotide sequence SEQ ID: 145 is located at position 81761 relative to the genome of Vaccinia Virus.

VGAM159 precursor RNA folds onto itself, forming VGAM159 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM159 folded precursor RNA into VGAM159 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM159 RNA is designated SEQ ID:494, and is provided hereinbelow with reference to the sequence listing part.

VGAM159 host target gene, herein designated VGAM HOST TARGET GENE, encodes a corresponding messenger RNA, VGAM159 host target RNA, herein designated VGAM HOST TARGET RNA. VGAM159 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM159 RNA, herein designated VGAM RNA, binds complementarily to one or more host target binding sites located in untranslated regions of VGAM159 host target RNA, herein designated VGAM HOST TARGET RNA. This complementary binding is due to the fact that the nucleotide sequence of VGAM159 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM159 RNA, herein designated VGAM RNA, may have a different number of host target binding sites in untranslated regions of a VGAM159 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM159 RNA, herein designated VGAM RNA, to host target binding sites on VGAM159 host target RNA, herein designated VGAM HOST TARGET RNA, such as BINDING SITE I, BINDING SITE II and BINDING SITE III, inhibits translation of VGAM159 host target RNA into VGAM159 host target protein, herein designated VGAM HOST TARGET PROTEIN. VGAM host target protein is therefore outlined by a broken line.

It is appreciated that VGAM159 host target gene, herein designated VGAM HOST TARGET GENE, in fact represents a plurality of VGAM159 host target genes. The mRNA of each one of this plurality of VGAM159 host target genes comprises one or more host target binding sites, each having a nucleotide sequence which is at least partly complementary to VGAM159 RNA, herein designated VGAM RNA, and which when bound by VGAM159 RNA causes inhibition of translation of respective one or more VGAM159 host target proteins.

It is further appreciated by one skilled in the art that the mode of translational inhibition illustrated by FIG. 1 with specific reference to translational inhibition exerted by VGAM159 gene, herein designated VGAM GENE, on one or more VGAM159 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM159 is inhibition of expression of host target genes, as part of a novel viral mechanism of attacking a host. Accordingly, utilities of VGAM159 include diagnosis, prevention and treatment of viral infection by Vaccinia Virus. Specific functions, and accordingly utilities, of VGAM159 correlate with, and may be deduced from, the identity of the host target genes which VGAM159 binds and inhibits, and the function of these host target genes, as elaborated hereinbelow.

Nucleotide sequences of the VGAM159 precursor RNA, herein designated VGAM PRECURSOR RNA, and of the ‘diced’ VGAM159 RNA, herein designated VGAM RNA, and a schematic representation of the secondary folding of VGAM159 folded precursor RNA, herein designated VGAM FOLDED PRECURSOR RNA, of VGAM159 are further described hereinbelow with reference to Table 1.

Nucleotide sequences of host target binding sites, such as BINDING SITE-I, BINDING SITE-II and BINDING SITE-III of FIG. 1, found on VGAM159 host target RNA, and schematic representation of the complementarity of each of these host target binding sites to VGAM159 RNA, herein designated VGAM RNA, are described hereinbelow with reference to Table 2.

As mentioned hereinabove with reference to FIG. 1, a function of VGAM159 gene, herein designated VGAM is inhibition of expression of VGAM159 target genes. It is appreciated that specific functions, and accordingly utilities, of VGAM159 correlate with, and may be deduced from, the identity of the target genes which VGAM159 binds and inhibits, and the function of these target genes, as elaborated hereinbelow.

Desmoglein 3 (pemphigus vulgaris antigen) (DSG3, Accession NM_{—}001944) is a VGAM159 host target gene. DSG3 BINDING SITE is HOST TARGET binding site found in the 3′ untranslated region of mRNA encoded by DSG3, corresponding to a HOST TARGET binding site such as BINDING SITE I, BINDING SITE II or BINDING SITE III. Table 2 illustrates the complementarity of the nucleotide sequences of DSG3 BINDING SITE, designated SEQ ID:873, to the nucleotide sequence of VGAM159 RNA, herein designated VGAM RNA, also designated SEQ ID:494.

A function of VGAM159 is therefore inhibition of Desmoglein 3 (pemphigus vulgaris antigen) (DSG3, Accession NM_{—}001944), a gene which is involved in the interaction of plaque proteins and intermediate filaments. Accordingly, utilities of VGAM159 include diagnosis, prevention and treatment of diseases and clinical conditions associated with DSG3. The function of DSG3 has been established by previous studies. Pemphigus vulgaris (PV; 169610) and pemphigus foliaceus (PF) are autoimmune diseases of the skin which have as target antigens 2 different members of the desmoglein sub family of the desmosomal cadherins: pemphigus vulgaris antigen (PVA, or desmoglein-3) in the case of PV and desmoglein I in the case of PF (Amagai et al., 1991). In pemphigus vulgaris, autoantibodies against PVA, a keratinocyte cell surface 130-kD glycoprotein, cause loss of cell-cell adhesion. Amagai et al. (1991) used affinity-purified pemphigus vulgaris IgG to isolate cDNA, containing the entire coding sequence for PVA, from human keratinocyte expression libraries. Northern blot analysis indicated PV mRNA expression only in stratified squamous epithelia. The deduced amino acid sequence of PVA shows significant homology with members of the cadherin family of Ca(2+)-dependent cell adhesion molecules, most markedly to desmoglein-1 (DSG1; 125670). Paraneoplastic pemphigus (PNP) is an autoimmune blistering disease that occurs in association with neoplasms such as non-Hodgkin lymphoma, chronic lymphatic leukemia, Castleman disease, thymomas, and poorly differentiated spindle cell sarcomas. Amagai et al. (1998) studied cell surface target antigens in paraneoplastic pemphigus. They focused on DSG3 and DSG1, the autoantigens of pemphigus vulgaris and pemphigus foliaceus, respectively. Using ELISA with baculovirus-expressed recombinant DSGs, they found that all 25 PNP sera tested were positive against DSG3 and that 16 of the 25 were positive against DSG1. All of 12 PNP sera tested immunoprecipitated DSG3. Removal of anti-DSG3 autoantibodies by immunoadsorption eliminated the ability of PNP sera to induce cutaneous blisters in neonatal mice; anti-DSG3 autoantibodies purified from PNP sera induced cutaneous blisters in neonatal mice. Thus, DSG3 and DSG1 are the target antigens in PNP, and IgG autoantibodies against DSG3 in PNP sera play a pathogenic role in the loss of keratinocyte adhesion and blistering.

Full details of the abovementioned studies are described in the following publications, the disclosure of which are hereby incorporated by reference:
Amagai, M.; Klaus-Kovtun, V.; Stanley, J. R.: Autoantibodies against a novel epithelial cadherin in pemphigus vulgaris, a disease of cell adhesion. Cell 67: 869-877, 1991.; and
Amagai, M.; Nishikawa, T.; Nousari, H. C.; Anhalt, G. J.; Hashimoto, T.: Antibodies against desmoglein 3 (pemphigus vulgaris antigen) are present in sera from patients with paraneoplas.

Further studies establishing the function and utilities of DSG3 are found in John Hopkins OMIM database record ID 169615, and in sited publications numbered 1122-751, 112 and 1124 listed in the bibliography section hereinbelow, which are also hereby incorporated by reference. Lipin 1 (LPIN1, Accession XM_{—}041136) is another VGAM159 host target gene. LPIN1 BINDING SITE is HOST TARGET binding site found in the 3′ untranslated region of mRNA encoded by LPIN1, corresponding to a HOST TARGET binding site such as BINDING SITE I, BINDING SITE II or BINDING SITE III. Table 2 illustrates the complementarity of the nucleotide sequences of LPIN1 BINDING SITE, designated SEQ ID:2791, to the nucleotide sequence of VGAM159 RNA, herein designated VGAM RNA, also designated SEQ ID:494.

Another function of VGAM159 is therefore inhibition of Lipin 1 (LPIN1, Accession XM_{—}041136), a gene which is involved in adipocyte differenciation (by similarity). Accordingly, utilities of VGAM159 include diagnosis, prevention and treatment of diseases and clinical conditions associated with LPIN1. The function of LPIN1 has been established by previous studies. Mice carrying mutations in the fatty liver dystrophy (fld) gene have features of human lipodystrophy (Reue et al., 2000). In the human, lipodystrophy is a heterogeneous group of disorders characterized by loss of body fat, fatty liver, hypertriglyceridemia, and insulin resistance. Through positional cloning, Peterfy et al. (2001) isolated the gene responsible for fatty liver dystrophy in mice and characterized 2 independent mutant alleles of the fld gene. They designated the gene Lpin1 and named the novel nuclear protein which it encodes lipin. Through database searches, Peterfy et al. (2001) identified several mouse and human EST and genomic sequences with similarities to Lpin1. These included 2 Lpin1-related mouse genes (Lpin2 and Lpin3) and 3 human homologs (LPIN1, LPIN2 (OMIM Ref. No. 605519), and LPIN3 (OMIM Ref. No. 605520)). LPIN1 is identical to the KIAA0188 gene identified by Nagase et al. (1996). Consistent with the observed reduction of adipose tissue mass in fld mice, wildtype Lpin1 mRNA was expressed at high levels in adipose tissue and was induced during differentiation of 3T3-L1 preadipocytes. The results indicated that lipin is required for normal adipose tissue development, and provided a candidate gene for human lipodystrophy Cao and Hegele (2002) sequenced the 21 exons of the LPIN1 gene in lipodystrophy patients who had no mutations in known lipodystrophy genes, and also in normal control subjects. They found no rare LPIN1 coding sequence variants that were exclusive to patients with lipodystrophy. However, they found 4 single nucleotide polymorphisms (SNPs).

Full details of the abovementioned studies are described in the following publications, the disclosure of which are hereby incorporated by reference:
Reue, K.; Xu, P.; Wang, X.-P.; Slavin, B. G.: Adipose tissue deficiency, glucose intolerance, and increased atherosclerosis result from mutation in the mouse fatty liver dystrophy (fld) gene. J. Lipid Res. 41: 1067-1076, 2000.; and
Cao, H.; Hegele, R. A.: Identification of single-nucleotide polymorphisms in the human LPIN1 gene. J. Hum. Genet. 47: 370-372, 2002.

Further studies establishing the function and utilities of LPIN1 are found in John Hopkins OMIM database record ID 605518, and in sited publications numbered 149 and 2639-1500 listed in the bibliography section hereinbelow, which are also hereby incorporated by reference. Transient Receptor Potential Cation Channel, Sub family M, Member 1 (TRPM1, Accession NM_{—}002420) is another VGAM159 host target gene. TRPM1 BINDING SITE is HOST TARGET binding site found in the 3′ untranslated region of mRNA encoded by TRPM1, corresponding to a HOST TARGET binding site such as BINDING SITE I, BINDING SITE II or BINDING SITE III. Table 2 illustrates the complementarity of the nucleotide sequences of TRPM1 BINDING SITE, designated SEQ ID:928, to the nucleotide sequence of VGAM159 RNA, herein designated VGAM RNA, also designated SEQ ID:494.

Another function of VGAM159 is therefore inhibition of Transient Receptor Potential Cation Channel, Sub family M, Member 1 (TRPM1, Accession NM_{—}002420), a gene which is suggested to mediate calcium entry. Accordingly, utilities of VGAM159 include diagnosis, prevention and treatment of diseases and clinical conditions associated with TRPM1. The function of TRPM1 has been established by previous studies. Hunter et al. (1998) cloned the human melastatin cDNA from a retina cDNA library. The gene encodes a 1,533-amino acid polypeptide with homology to members of the transient receptor potential (Trp) family of calcium channels (see OMIM Ref. No. TRPC1; 602343). They also cloned the mouse melastatin genomic region and found that the promoter contains 4 consensus binding sites for the microphthalmia-associated transcription factor (MITF; 156845). One of these binding sites is an M box, a motif shared by the tyrosinase pigmentation genes (see OMIM Ref. No. TYRP1; 115501). Xu et al. (2001) found that TRPM1 mediates Ca(2+) entry when expressed in HEK293 cells. They found that a short form of TRPM1 interacts directly with and suppresses the activity of full-length TRPM1, possibly by inhibiting translocation of the full-length form to the plasma membrane. Using differential display analysis, Fang and Setaluri (2000) found TRPM1 among genes overexpressed in pigmented metastatic human melanoma cells treated with the differentiation inducer hexamethylene bisacetamide (HMBA). They found multiple short transcripts, from both the 5-prime and 3-prime ends of TRPM1, in melanocytes and pigmented metastatic melanoma cell lines. They found the full-length 5.4-kb transcript only in melanocytes. Northern blot and RT-PCR analysis demonstrated that HMBA treatment up-regulates expression of the full-length and a 5-prime short form of TRPM1.

Full details of the abovementioned studies are described in the following publications, the disclosure of which are hereby incorporated by reference:
Hunter, J. J.; Shao, J.; Smutko, J. S.; Dussault, B. J.; Nagle, D. L.; Woolf, E. A.; Holmgren, L. M.; Moore, K. J.; Shyjan, A. W.: Chromosomal localization and genomic characterization of the mouse melastatin gene (Mlsn1). Genomics 54: 116-123, 1998.; and
Xu, X. Z.; Moebius, F.; Gill, D. L.; Montell, C.: Regulation of melastatin, a TRP-related protein, through interaction with a cytoplasmic isoform. Proc. Nat. Acad. Sci. 98: 10692-10697, 20.

Further studies establishing the function and utilities of TRPM1 are found in John Hopkins OMIM database record ID 603576, and in sited publications numbered 1221-1224 listed in the bibliography section hereinbelow, which are also hereby incorporated by reference. DEPP (Accession NM_{—}007021) is another VGAM159 host target gene. DEPP BINDING SITE is HOST TARGET binding site found in the 3′ untranslated region of mRNA encoded by DEPP, corresponding to a HOST TARGET binding site such as BINDING SITE I, BINDING SITE II or BINDING SITE III. Table 2 illustrates the complementarity of the nucleotide sequences of DEPP BINDING SITE, designated SEQ ID:1347, to the nucleotide sequence of VGAM159 RNA, herein designated VGAM RNA, also designated SEQ ID:494.

Another function of VGAM159 is therefore inhibition of DEPP (Accession NM_{—}007021). Accordingly, utilities of VGAM159 include diagnosis, prevention and treatment of diseases and clinical conditions associated with DEPP. FLJ20668 (Accession NM_{—}017923) is another VGAM159 host target gene. FLJ20668 BINDING SITE is HOST TARGET binding site found in the 3′ untranslated region of mRNA encoded by FLJ20668, corresponding to a HOST TARGET binding site such as BINDING SITE I, BINDING SITE II or BINDING SITE III. Table 2 illustrates the complementarity of the nucleotide sequences of FLJ20668 BINDING SITE, designated SEQ ID:1764, to the nucleotide sequence of VGAM159 RNA, herein designated VGAM RNA, also designated SEQ ID:494.

Another function of VGAM159 is therefore inhibition of FLJ20668 (Accession NM_{—}017923). Accordingly, utilities of VGAM159 include diagnosis, prevention and treatment of diseases and clinical conditions associated with FLJ20668. KIAA1524 (Accession XM_{—}056015) is another VGAM159 host target gene. KIAA1524 BINDING SITE is HOST TARGET binding site found in the 5′ untranslated region of mRNA encoded by KIAA1524, corresponding to a HOST TARGET binding site such as BINDING SITE I, BINDING SITE II or BINDING SITE III. Table 2 illustrates the complementarity of the nucleotide sequences of KIAA1524 BINDING SITE, designated SEQ ID:2975, to the nucleotide sequence of VGAM159 RNA, herein designated VGAM RNA, also designated SEQ ID:494.

Another function of VGAM159 is therefore inhibition of KIAA1524 (Accession XM_{—}056015). Accordingly, utilities of VGAM159 include diagnosis, prevention and treatment of diseases and clinical conditions associated with KIAA1524. PRO1617 (Accession NM_{—}018587) is another VGAM159 host target gene. PRO1617 BINDING SITE is HOST TARGET binding site found in the 5′ untranslated region of mRNA encoded by PRO1617, corresponding to a HOST TARGET binding site such as BINDING SITE I, BINDING SITE II or BINDING SITE III. Table 2 illustrates the complementarity of the nucleotide sequences of PRO1617 BINDING SITE, designated SEQ ID:1850, to the nucleotide sequence of VGAM159 RNA, herein designated VGAM RNA, also designated SEQ ID:494.

Another function of VGAM159 is therefore inhibition of PRO1617 (Accession NM_{—}018587). Accordingly, utilities of VGAM159 include diagnosis, prevention and treatment of diseases and clinical conditions associated with PRO1617. SDF1 (Accession XM_{—}165565) is another VGAM159 host target gene. SDF1 BINDING SITE is HOST TARGET binding site found in the 3′ untranslated region of mRNA encoded by SDF1, corresponding to a HOST TARGET binding site such as BINDING SITE I, BINDING SITE II or BINDING SITE III. Table 2 illustrates the complementarity of the nucleotide sequences of SDF1 BINDING SITE, designated SEQ ID:3502, to the nucleotide sequence of VGAM159 RNA, herein designated VGAM RNA, also designated SEQ ID:494.

Another function of VGAM159 is therefore inhibition of SDF1 (Accession XM_{—}165565). Accordingly, utilities of VGAM159 include diagnosis, prevention and treatment of diseases and clinical conditions associated with SDF1. LOC130074 (Accession XM_{—}072228) is another VGAM159 host target gene. LOC130074 BINDING SITE is HOST TARGET binding site found in the 3′ untranslated region of mRNA encoded by LOC130074, corresponding to a HOST TARGET binding site such as BINDING SITE I, BINDING SITE II or BINDING SITE III. Table 2 illustrates the complementarity of the nucleotide sequences of LOC130074 BINDING SITE, designated SEQ ID:3043, to the nucleotide sequence of VGAM159 RNA, herein designated VGAM RNA, also designated SEQ ID:494.

Another function of VGAM159 is therefore inhibition of LOC130074 (Accession XM_{—}072228). Accordingly, utilities of VGAM159 include diagnosis, prevention and treatment of diseases and clinical conditions associated with LOC130074. LOC146952 (Accession XM_{—}097138) is another VGAM159 host target gene. LOC146952 BINDING SITE is HOST TARGET binding site found in the 5′ untranslated region of mRNA encoded by LOC146952, corresponding to a HOST TARGET binding site such as BINDING SITE I, BINDING SITE II or BINDING SITE III. Table 2 illustrates the complementarity of the nucleotide sequences of LOC146952 BINDING SITE, designated SEQ ID:3287, to the nucleotide sequence of VGAM159 RNA, herein designated VGAM RNA, also designated SEQ ID:494.

Another function of VGAM159 is therefore inhibition of LOC146952 (Accession XM_{—}097138). Accordingly, utilities of VGAM159 include diagnosis, prevention and treatment of diseases and clinical conditions associated with LOC146952. LOC157983 (Accession XM_{—}088433) is another VGAM159 host target gene. LOC157983 BINDING SITE is HOST TARGET binding site found in the 3′ untranslated region of mRNA encoded by LOC157983, corresponding to a HOST TARGET binding site such as BINDING SITE I, BINDING SITE II or BINDING SITE III. Table 2 illustrates the complementarity of the nucleotide sequences of LOC157983 BINDING SITE, designated SEQ ID:3208, to the nucleotide sequence of VGAM159 RNA, herein designated VGAM RNA, also designated SEQ ID:494.

Another function of VGAM159 is therefore inhibition of LOC157983 (Accession XM_{—}088433). Accordingly, utilities of VGAM159 include diagnosis, prevention and treatment of diseases and clinical conditions associated with LOC157983. LOC203289 (Accession XM_{—}114672) is another VGAM159 host target gene. LOC203289 BINDING SITE is HOST TARGET binding site found in the 3′ untranslated region of mRNA encoded by LOC203289, corresponding to a HOST TARGET binding site such as BINDING SITE I, BINDING SITE II or BINDING SITE III. Table 2 illustrates the complementarity of the nucleotide sequences of LOC203289 BINDING SITE, designated SEQ ID:3463, to the nucleotide sequence of VGAM159 RNA, herein designated VGAM RNA, also designated SEQ ID:494.

Another function of VGAM159 is therefore inhibition of LOC203289 (Accession XM_{—}114672). Accordingly, utilities of VGAM159 include diagnosis, prevention and treatment of diseases and clinical conditions associated with LOC203289. LOC256544 (Accession XM_{—}171228) is another VGAM159 host target gene. LOC256544 BINDING SITE is HOST TARGET binding site found in the 3′ untranslated region of mRNA encoded by LOC256544, corresponding to a HOST TARGET binding site such as BINDING SITE I, BINDING SITE II or BINDING SITE III. Table 2 illustrates the complementarity of the nucleotide sequences of LOC256544 BINDING SITE, designated SEQ ID:3695, to the nucleotide sequence of VGAM159 RNA, herein designated VGAM RNA, also designated SEQ ID:494.

Another function of VGAM159 is therefore inhibition of LOC256544 (Accession XM_{—}171228). Accordingly, utilities of VGAM159 include diagnosis, prevention and treatment of diseases and clinical conditions associated with LOC256544. FIG. 1 further provides a conceptual description of a novel bioinformatically detected viral gene of the present invention, referred to here as Viral Genomic Address Messenger 160 (VGAM160) viral gene, which modulates expression of respective host target genes thereof, the function and utility of which host target genes is known in the art.

VGAM160 is a novel bioinformatically detected regulatory, non protein coding, viral micro RNA (miRNA) gene. The method by which VGAM160 was detected is described hereinabove with reference to FIGS. 1-8.

VGAM160 gene, herein designated VGAM GENE, is a viral gene contained in the genome of Vaccinia Virus. VGAM160 host target gene, herein designated VGAM HOST TARGET GENE, is a human gene contained in the human genome.

VGAM160 gene encodes a VGAM160 precursor RNA, herein designated VGAM PRECURSOR RNA. Similar to other miRNA genes, and unlike most ordinary genes, VGAM160 precursor RNA does not encode a protein. A nucleotide sequence identical or highly similar to the nucleotide sequence of VGAM160 precursor RNA is designated SEQ ID:146, and is provided hereinbelow with reference to the sequence listing part. Nucleotide sequence SEQ ID: 146 is located at position 81960 relative to the genome of Vaccinia Virus.

VGAM160 precursor RNA folds onto itself, forming VGAM160 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM160 folded precursor RNA into VGAM160 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM160 RNA is designated SEQ ID:495, and is provided hereinbelow with reference to the sequence listing part.

VGAM160 host target gene, herein designated VGAM HOST TARGET GENE, encodes a corresponding messenger RNA, VGAM160 host target RNA, herein designated VGAM HOST TARGET RNA. VGAM160 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM160 RNA, herein designated VGAM RNA, binds complementarily to one or more host target binding sites located in untranslated regions of VGAM160 host target RNA, herein designated VGAM HOST TARGET RNA. This complementary binding is due to the fact that the nucleotide sequence of VGAM160 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM160 RNA, herein designated VGAM RNA, may have a different number of host target binding sites in untranslated regions of a VGAM160 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM160 RNA, herein designated VGAM RNA, to host target binding sites on VGAM160 host target RNA, herein designated VGAM HOST TARGET RNA, such as BINDING SITE I, BINDING SITE II and BINDING SITE III, inhibits translation of VGAM160 host target RNA into VGAM160 host target protein, herein designated VGAM HOST TARGET PROTEIN. VGAM host target protein is therefore outlined by a broken line.

It is appreciated that VGAM160 host target gene, herein designated VGAM HOST TARGET GENE, in fact represents a plurality of VGAM160 host target genes. The mRNA of each one of this plurality of VGAM160 host target genes comprises one or more host target binding sites, each having a nucleotide sequence which is at least partly complementary to VGAM160 RNA, herein designated VGAM RNA, and which when bound by VGAM160 RNA causes inhibition of translation of respective one or more VGAM160 host target proteins.

It is further appreciated by one skilled in the art that the mode of translational inhibition illustrated by FIG. 1 with specific reference to translational inhibition exerted by VGAM160 gene, herein designated VGAM GENE, on one or more VGAM160 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM160 is inhibition of expression of host target genes, as part of a novel viral mechanism of attacking a host. Accordingly, utilities of VGAM160 include diagnosis, prevention and treatment of viral infection by Vaccinia Virus. Specific functions, and accordingly utilities, of VGAM160 correlate with, and may be deduced from, the identity of the host target genes which VGAM160 binds and inhibits, and the function of these host target genes, as elaborated hereinbelow.

Nucleotide sequences of the VGAM160 precursor RNA, herein designated VGAM PRECURSOR RNA, and of the ‘diced’ VGAM160 RNA, herein designated VGAM RNA, and a schematic representation of the secondary folding of VGAM160 folded precursor RNA, herein designated VGAM FOLDED PRECURSOR RNA, of VGAM160 are further described hereinbelow with reference to Table 1.

Nucleotide sequences of host target binding sites, such as BINDING SITE-I, BINDING SITE-II and BINDING SITE-III of FIG. 1, found on VGAM160 host target RNA, and schematic representation of the complementarity of each of these host target binding sites to VGAM160 RNA, herein designated VGAM RNA, are described hereinbelow with reference to Table 2.

As mentioned hereinabove with reference to FIG. 1, a function of VGAM160 gene, herein designated VGAM is inhibition of expression of VGAM160 target genes. It is appreciated that specific functions, and accordingly utilities, of VGAM160 correlate with, and may be deduced from, the identity of the target genes which VGAM160 binds and inhibits, and the function of these target genes, as elaborated hereinbelow.

APPL (Accession NM_{—}012096) is a VGAM160 host target gene. APPL BINDING SITE is HOST TARGET binding site found in the 3′ untranslated region of mRNA encoded by APPL, corresponding to a HOST TARGET binding site such as BINDING SITE I, BINDING SITE II or BINDING SITE III. Table 2 illustrates the complementarity of the nucleotide sequences of APPL BINDING SITE, designated SEQ ID:1405, to the nucleotide sequence of VGAM160 RNA, herein designated VGAM RNA, also designated SEQ ID:495.

A function of VGAM160 is therefore inhibition of APPL (Accession NM_{—}012096). Accordingly, utilities of VGAM160 include diagnosis, prevention and treatment of diseases and clinical conditions associated with APPL. Coagulation Factor VIII, Procoagulant Component (hemophilia A) (F8, Accession NM_{—}000132) is another VGAM160 host target gene. F8 BINDING SITE is HOST TARGET binding site found in the 3′ untranslated region of mRNA encoded by F8, corresponding to a HOST TARGET binding site such as BINDING SITE I, BINDING SITE II or BINDING SITE III. Table 2 illustrates the complementarity of the nucleotide sequences of F8 BINDING SITE, designated SEQ ID:708, to the nucleotide sequence of VGAM160 RNA, herein designated VGAM RNA, also designated SEQ ID:495.

Another function of VGAM160 is therefore inhibition of Coagulation Factor VIII, Procoagulant Component (hemophilia A) (F8, Accession NM_{—}000132). Accordingly, utilities of VGAM160 include diagnosis, prevention and treatment of diseases and clinical conditions associated with F8. RAB6A, Member RAS Oncogene Family (RAB6A, Accession NM_{—}002869) is another VGAM160 host target gene. RAB6A BINDING SITE is HOST TARGET binding site found in the 3′ untranslated region of mRNA encoded by RAB6A, corresponding to a HOST TARGET binding site such as BINDING SITE I, BINDING SITE II or BINDING SITE III. Table 2 illustrates the complementarity of the nucleotide sequences of RAB6A BINDING SITE, designated SEQ ID:966, to the nucleotide sequence of VGAM160 RNA, herein designated VGAM RNA, also designated SEQ ID:495.

Another function of VGAM160 is therefore inhibition of RAB6A, Member RAS Oncogene Family (RAB6A, Accession NM_{—}002869), a gene which is involved in protein trafficking. Accordingly, utilities of VGAM160 include diagnosis, prevention and treatment of diseases and clinical conditions associated with RAB6A. The function of RAB6A has been established by previous studies. The mammalian RAB proteins show striking similarities to the S. cerevisiae YPT1 and SEC4 proteins, Ras-related GTP-binding proteins involved in the regulation of secretion. Zahraoui et al. (1989) isolated cDNAs encoding RAB6 and several other human RAB proteins. See RAB5A (OMIM Ref. No. 179512). The predicted human RAB6 protein contains 208 amino acids. Northern blot analysis revealed that the RAB6 gene was expressed as a 3.6-kb mRNA in a human fibroblast cell line. By in situ hybridization, Rousseau-Merck et al. (1991) assigned the RAB6 gene to 2q14-q21.

Full details of the abovementioned studies are described in the following publications, the disclosure of which are hereby incorporated by reference:
Rousseau-Merck, M.-F.; Zahraoui, A.; Touchot, N.; Tavitian, A.; Berger, R.: Chromosome assignment of four RAS-related RAB genes. Hum. Genet. 86: 350-354, 1991.; and
Zahraoui, A.; Touchot, N.; Chardin, P.; Tavitian, A.: The human rab genes encode a family of GTP-binding proteins related to yeast YPT1 and SEC4 products involved in secretion. J. Biol.

Further studies establishing the function and utilities of RAB6A are found in John Hopkins OMIM database record ID 179513, and in sited publications numbered 60 and 626 listed in the bibliography section hereinbelow, which are also hereby incorporated by reference. Ring Finger Protein 14 (RNF14, Accession NM_{—}004290) is another VGAM160 host target gene. RNF14 BINDING SITE is HOST TARGET binding site found in the 3′ untranslated region of mRNA encoded by RNF14, corresponding to a HOST TARGET binding site such as BINDING SITE I, BINDING SITE II or BINDING SITE III. Table 2 illustrates the complementarity of the nucleotide sequences of RNF14 BINDING SITE, designated SEQ ID: 1097, to the nucleotide sequence of VGAM160 RNA, herein designated VGAM RNA, also designated SEQ ID:495.

Another function of VGAM160 is therefore inhibition of Ring Finger Protein 14 (RNF14, Accession NM_{—}004290), a gene which associates with the androgen receptor (AR); functions as a transcriptional coactivator. Accordingly, utilities of VGAM160 include diagnosis, prevention and treatment of diseases and clinical conditions associated with RNF14. The function of RNF14 has been established by previous studies. The RING finger motif is a unique zinc-chelating domain involved in mediating protein-DNA and protein-protein interactions. Using the sequence of the partial cDNA clone HFB30 isolated by Ueki et al. (1998) to screen a human fetal brain cDNA library, Ueki et al. (1999) cloned the full-length cDNA, which encoded a novel ring finger protein, RNF14. The deduced 474-amino acid protein has a calculated molecular mass of approximately 53 kD. RT-PCR analysis revealed ubiquitous expression of RNF14 in a wide variety of human tissues. Kang et al. (1999) independently cloned RNF14, which they called ARA54 (androgen receptor-associated protein-54), by a yeast 2-hybrid screen of a prostate cDNA library. Northern blot analysis detected a major 3-kb transcript, with highest expression in testis, followed by thymus, spleen, colon, prostate, and uterus. Low expression was detected in small intestine and blood leukocytes. The RNF14 transcript was also strongly detected in 2 other prostate cell lines. A second transcript of 2 kb was detected in testis only. Kang et al. (1999) demonstrated that RNF14 can function as a coactivator for androgen-dependent transcription on both wildtype and mutant androgen receptor (OMIM Ref. No. 313700). They also showed that in the presence of a certain amount of 17-beta-estradiol or hydroxyflutamide, the transcriptional activity of a specific AR mutant was significantly enhanced, whereas that of wildtype and another AR mutant was not. The authors suggested that both RNF14 and the positions of the AR mutation might contribute to the specificity of AR-mediated transactivation. Ueki et al. (1999) determined that the RNF14 gene contains 9 exons and spans approximately 20 kb of genomic DNA. By somatic cell hybrid and radiation hybrid analyses, Ueki et al. (1999) mapped the RNF14 gene to chromosome 5q23.3-q31.1.

Full details of the abovementioned studies are described in the following publications, the disclosure of which are hereby incorporated by reference:
Ueki, N.; Seki, N.; Yano, K.; Masuho, Y.; Saito, T.; Muramatsu, M.: Isolation and characterization of a novel human gene (HFB30) which encodes a protein with a RING finger motif. Biochim. Biophys. Acta 232-236, 1999.; and
Kang, H.-Y.; Yeh, S.; Fujimoto, N.; Chang, C.: Cloning and characterization of human prostate coactivator ARA54, a novel protein that associates with the androgen receptor. J. Biol. Che.

Further studies establishing the function and utilities of RNF14 are found in John Hopkins OMIM database record ID 605675, and in sited publications numbered 1993-219 listed in the bibliography section hereinbelow, which are also hereby incorporated by reference. SART2 (Accession NM_{—}013352) is another VGAM160 host target gene. SART2 BINDING SITE is HOST TARGET binding site found in the 3′ untranslated region of mRNA encoded by SART2, corresponding to a HOST TARGET binding site such as BINDING SITE I, BINDING SITE II or BINDING SITE III. Table 2 illustrates the complementarity of the nucleotide sequences of SART2 BINDING SITE, designated SEQ ID:1443, to the nucleotide sequence of VGAM160 RNA, herein designated VGAM RNA, also designated SEQ ID:495.

Another function of VGAM160 is therefore inhibition of SART2 (Accession NM_{—}013352). Accordingly, utilities of VGAM160 include diagnosis, prevention and treatment of diseases and clinical conditions associated with SART2. Early Endosome Antigen 1, 162 kD (EEA1, Accession NM_{—}003566) is another VGAM160 host target gene. EEA1 BINDING SITE is HOST TARGET binding site found in the 3′ untranslated region of mRNA encoded by EEA1, corresponding to a HOST TARGET binding site such as BINDING SITE I, BINDING SITE II or BINDING SITE III. Table 2 illustrates the complementarity of the nucleotide sequences of EEA1 BINDING SITE, designated SEQ ID:1035, to the nucleotide sequence of VGAM160 RNA, herein designated VGAM RNA, also designated SEQ ID:495.

Another function of VGAM160 is therefore inhibition of Early Endosome Antigen 1, 162 kD (EEA1, Accession NM_{—}003566). Accordingly, utilities of VGAM160 include diagnosis, prevention and treatment of diseases and clinical conditions associated with EEA1. FLJ10980 (Accession XM_{—}035527) is another VGAM160 host target gene. FLJ10980 BINDING SITE is HOST TARGET binding site found in the 3′ untranslated region of mRNA encoded by FLJ10980, corresponding to a HOST TARGET binding site such as BINDING SITE I, BINDING SITE II or BINDING SITE III. Table 2 illustrates the complementarity of the nucleotide sequences of FLJ10980 BINDING SITE, designated SEQ ID:2697, to the nucleotide sequence of VGAM160 RNA, herein designated VGAM RNA, also designated SEQ ID:495.

Another function of VGAM160 is therefore inhibition of FLJ10980 (Accession XM_{—}035527). Accordingly, utilities of VGAM160 include diagnosis, prevention and treatment of diseases and clinical conditions associated with FLJ10980. FLJ22794 (Accession XM_{—}166220) is another VGAM160 host target gene. FLJ22794 BINDING SITE is HOST TARGET binding site found in the 3′ untranslated region of mRNA encoded by FLJ22794, corresponding to a HOST TARGET binding site such as BINDING SITE I, BINDING SITE II or BINDING SITE III. Table 2 illustrates the complementarity of the nucleotide sequences of FLJ22794 BINDING SITE, designated SEQ ID:3532, to the nucleotide sequence of VGAM160 RNA, herein designated VGAM RNA, also designated SEQ ID:495.

Another function of VGAM160 is therefore inhibition of FLJ22794 (Accession XM_{—}166220). Accordingly, utilities of VGAM160 include diagnosis, prevention and treatment of diseases and clinical conditions associated with FLJ22794. KIAA1328 (Accession XM_{—}029429) is another VGAM160 host target gene. KIAA1328 BINDING SITE is HOST TARGET binding site found in the 3′ untranslated region of mRNA encoded by KIAA1328, corresponding to a HOST TARGET binding site such as BINDING SITE I, BINDING SITE II or BINDING SITE III. Table 2 illustrates the complementarity of the nucleotide sequences of KIAA1328 BINDING SITE, designated SEQ ID:2601, to the nucleotide sequence of VGAM160 RNA, herein designated VGAM RNA, also designated SEQ ID:495.

Another function of VGAM160 is therefore inhibition of KIAA1328 (Accession XM_{—}029429). Accordingly, utilities of VGAM160 include diagnosis, prevention and treatment of diseases and clinical conditions associated with KIAA1328. PRO0902 (Accession NM_{—}053057) is another VGAM160 host target gene. PRO0902 BINDING SITE is HOST TARGET binding site found in the 3′ untranslated region of mRNA encoded by PRO0902, corresponding to a HOST TARGET binding site such as BINDING SITE I, BINDING SITE II or BINDING SITE III. Table 2 illustrates the complementarity of the nucleotide sequences of PRO0902 BINDING SITE, designated SEQ ID:2357, to the nucleotide sequence of VGAM160 RNA, herein designated VGAM RNA, also designated SEQ ID:495.

Another function of VGAM160 is therefore inhibition of PRO0902 (Accession NM_{—}053057). Accordingly, utilities of VGAM160 include diagnosis, prevention and treatment of diseases and clinical conditions associated with PRO0902. RAS Guanyl Releasing Protein 4 (RASGRP4, Accession NM_{—}052949) is another VGAM160 host target gene. RASGRP4 BINDING SITE is HOST TARGET binding site found in the 3′ untranslated region of mRNA encoded by RASGRP4, corresponding to a HOST TARGET binding site such as BINDING SITE I, BINDING SITE II or BINDING SITE III. Table 2 illustrates the complementarity of the nucleotide sequences of RASGRP4 BINDING SITE, designated SEQ ID:2348, to the nucleotide sequence of VGAM160 RNA, herein designated VGAM RNA, also designated SEQ ID:495.

Another function of VGAM160 is therefore inhibition of RAS Guanyl Releasing Protein 4 (RASGRP4, Accession NM_{—}052949). Accordingly, utilities of VGAM160 include diagnosis, prevention and treatment of diseases and clinical conditions associated with RASGRP4. WW Domain Binding Protein 4 (formin binding protein 21) (WBP4, Accession NM_{—}007187) is another VGAM160 host target gene. WBP4 BINDING SITE is HOST TARGET binding site found in the 3′ untranslated region of mRNA encoded by WBP4, corresponding to a HOST TARGET binding site such as BINDING SITE I, BINDING SITE II or BINDING SITE III. Table 2 illustrates the complementarity of the nucleotide sequences of WBP4 BINDING SITE, designated SEQ ID:1363, to the nucleotide sequence of VGAM160 RNA, herein designated VGAM RNA, also designated SEQ ID:495.

Another function of VGAM160 is therefore inhibition of WW Domain Binding Protein 4 (formin binding protein 21) (WBP4, Accession NM_{—}007187). Accordingly, utilities of VGAM160 include diagnosis, prevention and treatment of diseases and clinical conditions associated with WBP4. LOC116150 (Accession NM_{—}138459) is another VGAM160 host target gene. LOC116150 BINDING SITE is HOST TARGET binding site found in the 3′ untranslated region of mRNA encoded by LOC116150, corresponding to a HOST TARGET binding site such as BINDING SITE I, BINDING SITE II or BINDING SITE III. Table 2 illustrates the complementarity of the nucleotide sequences of LOC116150 BINDING SITE, designated SEQ ID:2443, to the nucleotide sequence of VGAM160 RNA, herein designated VGAM RNA, also designated SEQ ID:495.

Another function of VGAM160 is therefore inhibition of LOC116150 (Accession NM_{—}138459). Accordingly, utilities of VGAM160 include diagnosis, prevention and treatment of diseases and clinical conditions associated with LOC116150. LOC164200 (Accession XM_{—}092690) is another VGAM160 host target gene. LOC164200 BINDING SITE is HOST TARGET binding site found in the 5′ untranslated region of mRNA encoded by LOC164200, corresponding to a HOST TARGET binding site such as BINDING SITE I, BINDING SITE II or BINDING SITE III. Table 2 illustrates the complementarity of the nucleotide sequences of LOC164200 BINDING SITE, designated SEQ ID:3248, to the nucleotide sequence of VGAM160 RNA, herein designated VGAM RNA, also designated SEQ ID:495.

Another function of VGAM160 is therefore inhibition of LOC164200 (Accession XM_{—}092690). Accordingly, utilities of VGAM160 include diagnosis, prevention and treatment of diseases and clinical conditions associated with LOC164200. LOC51320 (Accession NM_{—}016626) is another VGAM160 host target gene. LOC51320 BINDING SITE is HOST TARGET binding site found in the 3′ untranslated region of mRNA encoded by LOC51320, corresponding to a HOST TARGET binding site such as BINDING SITE I, BINDING SITE II or BINDING SITE III. Table 2 illustrates the complementarity of the nucleotide sequences of LOC51320 BINDING SITE, designated SEQ ID:1702, to the nucleotide sequence of VGAM160 RNA, herein designated VGAM RNA, also designated SEQ ID:495.

Another function of VGAM160 is therefore inhibition of LOC51320 (Accession NM_{—}016626). Accordingly, utilities of VGAM160 include diagnosis, prevention and treatment of diseases and clinical conditions associated with LOC51320. FIG. 1 further provides a conceptual description of a novel bioinformatically detected viral gene of the present invention, referred to here as Viral Genomic Address Messenger 161 (VGAM161) viral gene, which modulates expression of respective host target genes thereof, the function and utility of which host target genes is known in the art.

VGAM161 is a novel bioinformatically detected regulatory, non protein coding, viral micro RNA (miRNA) gene. The method by which VGAM161 was detected is described hereinabove with reference to FIGS. 1-8.

VGAM161 gene, herein designated VGAM GENE, is a viral gene contained in the genome of Vaccinia Virus. VGAM161 host target gene, herein designated VGAM HOST TARGET GENE, is a human gene contained in the human genome.

VGAM161 gene encodes a VGAM161 precursor RNA, herein designated VGAM PRECURSOR RNA. Similar to other miRNA genes, and unlike most ordinary genes, VGAM161 precursor RNA does not encode a protein. A nucleotide sequence identical or highly similar to the nucleotide sequence of VGAM161 precursor RNA is designated SEQ ID:147, and is provided hereinbelow with reference to the sequence listing part. Nucleotide sequence SEQ ID: 147 is located at position 81428 relative to the genome of Vaccinia Virus.

VGAM161 precursor RNA folds onto itself, forming VGAM161 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM161 folded precursor RNA into VGAM161 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM161 RNA is designated SEQ ID:496, and is provided hereinbelow with reference to the sequence listing part.

VGAM161 host target gene, herein designated VGAM HOST TARGET GENE, encodes a corresponding messenger RNA, VGAM161 host target RNA, herein designated VGAM HOST TARGET RNA. VGAM161 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM161 RNA, herein designated VGAM RNA, binds complementarily to one or more host target binding sites located in untranslated regions of VGAM161 host target RNA, herein designated VGAM HOST TARGET RNA. This complementary binding is due to the fact that the nucleotide sequence of VGAM161 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM161 RNA, herein designated VGAM RNA, may have a different number of host target binding sites in untranslated regions of a VGAM161 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM161 RNA, herein designated VGAM RNA, to host target binding sites on VGAM161 host target RNA, herein designated VGAM HOST TARGET RNA, such as BINDING SITE I, BINDING SITE II and BINDING SITE III, inhibits translation of VGAM161 host target RNA into VGAM161 host target protein, herein designated VGAM HOST TARGET PROTEIN. VGAM host target protein is therefore outlined by a broken line.

It is appreciated that VGAM161 host target gene, herein designated VGAM HOST TARGET GENE, in fact represents a plurality of VGAM161 host target genes. The mRNA of each one of this plurality of VGAM161 host target genes comprises one or more host target binding sites, each having a nucleotide sequence which is at least partly complementary to VGAM161 RNA, herein designated VGAM RNA, and which when bound by VGAM161 RNA causes inhibition of translation of respective one or more VGAM161 host target proteins.

It is further appreciated by one skilled in the art that the mode of translational inhibition illustrated by FIG. 1 with specific reference to translational inhibition exerted by VGAM161 gene, herein designated VGAM GENE, on one or more VGAM161 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM161 is inhibition of expression of host target genes, as part of a novel viral mechanism of attacking a host. Accordingly, utilities of VGAM161 include diagnosis, prevention and treatment of viral infection by Vaccinia Virus. Specific functions, and accordingly utilities, of VGAM161 correlate with, and may be deduced from, the identity of the host target genes which VGAM161 binds and inhibits, and the function of these host target genes, as elaborated hereinbelow.

Nucleotide sequences of the VGAM161 precursor RNA, herein designated VGAM PRECURSOR RNA, and of the ‘diced’ VGAM161 RNA, herein designated VGAM RNA, and a schematic representation of the secondary folding of VGAM161 folded precursor RNA, herein designated VGAM FOLDED PRECURSOR RNA, of VGAM161 are further described hereinbelow with reference to Table 1.

Nucleotide sequences of host target binding sites, such as BINDING SITE-I, BINDING SITE-II and BINDING SITE-III of FIG. 1, found on VGAM161 host target RNA, and schematic representation of the complementarity of each of these host target binding sites to VGAM161 RNA, herein designated VGAM RNA, are described hereinbelow with reference to Table 2.

As mentioned hereinabove with reference to FIG. 1, a function of VGAM161 gene, herein designated VGAM is inhibition of expression of VGAM161 target genes. It is appreciated that specific functions, and accordingly utilities, of VGAM161 correlate with, and may be deduced from, the identity of the target genes which VGAM161 binds and inhibits, and the function of these target genes, as elaborated hereinbelow.

Selectin P Ligand (SELPLG, Accession XM_{—}006867) is a VGAM161 host target gene. SELPLG BINDING SITE is HOST TARGET binding site found in the 3′ untranslated region of mRNA encoded by SELPLG, corresponding to a HOST TARGET binding site such as BINDING SITE I, BINDING SITE II or BINDING SITE III. Table 2 illustrates the complementarity of the nucleotide sequences of SELPLG BINDING SITE, designated SEQ ID:2539, to the nucleotide sequence of VGAM161 RNA, herein designated VGAM RNA, also designated SEQ ID:496.

A function of VGAM161 is therefore inhibition of Selectin P Ligand (SELPLG, Accession XM_{—}006867), a gene which binds to p-, e- and 1-selectins, which mediates the tethering and rolling of neutrophils and t-lymphocytes on endothelial cells. Accordingly, utilities of VGAM161 include diagnosis, prevention and treatment of diseases and clinical conditions associated with SELPLG. The function of SELPLG has been established by previous studies. Human granulocyte ehrlichiosis (HGE) is a febrile tick-bone illness caused by an intracellular bacterium remarkable for its tropism for professionally phagocytic neutrophils. Herron et al. (2000) demonstrated that monoclonal antibodies against the P-selectin binding domain of the leukocyte P-selectin glycoprotein ligand PSGL1 prevented HGE cell binding and infection, as did enzymatic digestion of PSGL1. Furthermore, simultaneous neoexpression in non-susceptible cells of complementary DNAs for both PSGL1 and its modifying alpha-(1,3) fucosyltransferase, Fuc-TVII (FUT7), allowed binding and infection by HGE. Thus, the HGE bacterium specifically bound to fucosylated leukocyte PSGL1. Selection mimicry is likely central to the organism's unique ability to target and infect neutrophils. Selectin P ligand, or P-selectin glycoprotein ligand (OMIM Ref. No. PSGL-1), is the high affinity counter-receptor for P-selectin (SELP; 173610) on myeloid cells and stimulated T lymphocytes. As such, it plays a critical role in the tethering of these cells to activated platelets or endothelia expressing P-selectin. Veldman et al. (1995) cloned the SELPLG gene from a human placenta genomic DNA library and showed that a single intron of approximately 9 kb is located in the 5-prime untranslated region and that the complete coding region resides in exon 2. The organization of the gene, designated SELPLG, closely resembles that of CD43 (OMIM Ref. No. 182160) and the human platelet glycoprotein GpIb-alpha (OMIM Ref. No. 231200), both of which have an intron in the 5-prime-noncoding region, a long second exon containing the complete coding region, and TATA-less promoters.

Full details of the abovementioned studies are described in the following publications, the disclosure of which are hereby incorporated by reference:
Herron, M. J.; Nelson, C. M.; Larson, J.; Snapp, K. R.; Kansas, G. S.; Goodman, J. L.: Intracellular parasitism by the human granulocytic ehrlichiosis bacterium through the P-selectin ligand, PSGL-1. Science 288: 1653-1656, 2000.; and
Veldman, G. M.; Bean, K. M.; Cumming, D. A.; Eddy, R. L.; Sait, S. N. J.; Shows, T. B.: Genomic organization and chromosomal localization of the gene encoding human P-selectin glycop.

Further studies establishing the function and utilities of SELPLG are found in John Hopkins OMIM database record ID 600738, and in sited publications numbered 1718-1721 listed in the bibliography section hereinbelow, which are also hereby incorporated by reference. FLJ21144 (Accession NM_{—}022774) is another VGAM161 host target gene. FLJ21144 BINDING SITE is HOST TARGET binding site found in the 3′ untranslated region of mRNA encoded by FLJ21144, corresponding to a HOST TARGET binding site such as BINDING SITE I, BINDING SITE II or BINDING SITE III. Table 2 illustrates the complementarity of the nucleotide sequences of FLJ21144 BINDING SITE, designated SEQ ID:2007, to the nucleotide sequence of VGAM161 RNA, herein designated VGAM RNA, also designated SEQ ID:496.

Another function of VGAM161 is therefore inhibition of FLJ21144 (Accession NM_{—}022774). Accordingly, utilities of VGAM161 include diagnosis, prevention and treatment of diseases and clinical conditions associated with FLJ21144. KIAA0676 (Accession NM_{—}015043) is another VGAM161 host target gene. KIAA0676 BINDING SITE is HOST TARGET binding site found in the 3′ untranslated region of mRNA encoded by KIAA0676, corresponding to a HOST TARGET binding site such as BINDING SITE I, BINDING SITE II or BINDING SITE III. Table 2 illustrates the complementarity of the nucleotide sequences of KIAA0676 BINDING SITE, designated SEQ ID: 1603, to the nucleotide sequence of VGAM161 RNA, herein designated VGAM RNA, also designated SEQ ID:496.

Another function of VGAM161 is therefore inhibition of KIAA0676 (Accession NM_{—}015043). Accordingly, utilities of VGAM161 include diagnosis, prevention and treatment of diseases and clinical conditions associated with KIAA0676. KIAA1393 (Accession XM_{—}050793) is another VGAM161 host target gene. KIAA1393 BINDING SITE is HOST TARGET binding site found in the 3′ untranslated region of mRNA encoded by KIAA1393, corresponding to a HOST TARGET binding site such as BINDING SITE I, BINDING SITE II or BINDING SITE III. Table 2 illustrates the complementarity of the nucleotide sequences of KIAA1393 BINDING SITE, designated SEQ ID:2935, to the nucleotide sequence of VGAM161 RNA, herein designated VGAM RNA, also designated SEQ ID:496.

Another function of VGAM161 is therefore inhibition of KIAA1393 (Accession XM_{—}050793). Accordingly, utilities of VGAM161 include diagnosis, prevention and treatment of diseases and clinical conditions associated with KIAA1393. Ubinuclein 1 (UBN1, Accession NM_{—}016936) is another VGAM161 host target gene. UBN1 BINDING SITE is HOST TARGET binding site found in the 3′ untranslated region of mRNA encoded by UBN1, corresponding to a HOST TARGET binding site such as BINDING SITE I, BINDING SITE II or BINDING SITE III. Table 2 illustrates the complementarity of the nucleotide sequences of UBN1 BINDING SITE, designated SEQ ID:1716, to the nucleotide sequence of VGAM161 RNA, herein designated VGAM RNA, also designated SEQ ID:496.

Another function of VGAM161 is therefore inhibition of Ubinuclein 1 (UBN1, Accession NM_{—}016936). Accordingly, utilities of VGAM161 include diagnosis, prevention and treatment of diseases and clinical conditions associated with UBN1. FIG. 1 further provides a conceptual description of a novel bioinformatically detected viral gene of the present invention, referred to here as Viral Genomic Address Messenger 162 (VGAM162) viral gene, which modulates expression of respective host target genes thereof, the function and utility of which host target genes is known in the art.

VGAM162 is a novel bioinformatically detected regulatory, non protein coding, viral micro RNA (miRNA) gene. The method by which VGAM162 was detected is described hereinabove with reference to FIGS. 1-8.

VGAM162 gene, herein designated VGAM GENE, is a viral gene contained in the genome of Vaccinia Virus. VGAM162 host target gene, herein designated VGAM HOST TARGET GENE, is a human gene contained in the human genome.

VGAM162 gene encodes a VGAM162 precursor RNA, herein designated VGAM PRECURSOR RNA. Similar to other miRNA genes, and unlike most ordinary genes, VGAM162 precursor RNA does not encode a protein. A nucleotide sequence identical or highly similar to the nucleotide sequence of VGAM162 precursor RNA is designated SEQ ID:148, and is provided hereinbelow with reference to the sequence listing part. Nucleotide sequence SEQ ID: 148 is located at position 81204 relative to the genome of Vaccinia Virus.

VGAM162 precursor RNA folds onto itself, forming VGAM162 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM162 folded precursor RNA into VGAM162 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM162 RNA is designated SEQ ID:497, and is provided hereinbelow with reference to the sequence listing part.

VGAM162 host target gene, herein designated VGAM HOST TARGET GENE, encodes a corresponding messenger RNA, VGAM162 host target RNA, herein designated VGAM HOST TARGET RNA. VGAM162 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM162 RNA, herein designated VGAM RNA, binds complementarily to one or more host target binding sites located in untranslated regions of VGAM162 host target RNA, herein designated VGAM HOST TARGET RNA. This complementary binding is due to the fact that the nucleotide sequence of VGAM162 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM162 RNA, herein designated VGAM RNA, may have a different number of host target binding sites in untranslated regions of a VGAM162 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM162 RNA, herein designated VGAM RNA, to host target binding sites on VGAM162 host target RNA, herein designated VGAM HOST TARGET RNA, such as BINDING SITE I, BINDING SITE II and BINDING SITE III, inhibits translation of VGAM162 host target RNA into VGAM162 host target protein, herein designated VGAM HOST TARGET PROTEIN. VGAM host target protein is therefore outlined by a broken line.

It is appreciated that VGAM162 host target gene, herein designated VGAM HOST TARGET GENE, in fact represents a plurality of VGAM162 host target genes. The mRNA of each one of this plurality of VGAM162 host target genes comprises one or more host target binding sites, each having a nucleotide sequence which is at least partly complementary to VGAM162 RNA, herein designated VGAM RNA, and which when bound by VGAM162 RNA causes inhibition of translation of respective one or more VGAM162 host target proteins.

It is further appreciated by one skilled in the art that the mode of translational inhibition illustrated by FIG. 1 with specific reference to translational inhibition exerted by VGAM162 gene, herein designated VGAM GENE, on one or more VGAM162 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM162 is inhibition of expression of host target genes, as part of a novel viral mechanism of attacking a host. Accordingly, utilities of VGAM162 include diagnosis, prevention and treatment of viral infection by Vaccinia Virus. Specific functions, and accordingly utilities, of VGAM162 correlate with, and may be deduced from, the identity of the host target genes which VGAM162 binds and inhibits, and the function of these host target genes, as elaborated hereinbelow.

Nucleotide sequences of the VGAM162 precursor RNA, herein designated VGAM PRECURSOR RNA, and of the ‘diced’ VGAM162 RNA, herein designated VGAM RNA, and a schematic representation of the secondary folding of VGAM162 folded precursor RNA, herein designated VGAM FOLDED PRECURSOR RNA, of VGAM162 are further described hereinbelow with reference to Table 1.

Nucleotide sequences of host target binding sites, such as BINDING SITE-I, BINDING SITE-II and BINDING SITE-III of FIG. 1, found on VGAM162 host target RNA, and schematic representation of the complementarity of each of these host target binding sites to VGAM162 RNA, herein designated VGAM RNA, are described hereinbelow with reference to Table 2.

As mentioned hereinabove with reference to FIG. 1, a function of VGAM162 gene, herein designated VGAM is inhibition of expression of VGAM162 target genes. It is appreciated that specific functions, and accordingly utilities, of VGAM162 correlate with, and may be deduced from, the identity of the target genes which VGAM162 binds and inhibits, and the function of these target genes, as elaborated hereinbelow.

Cadherin, EGF LAG Seven-pass G-type Receptor 3 (flamingo homolog, Drosophila) (CELSR3, Accession NM_{—}001407) is a VGAM162 host target gene. CELSR3 BINDING SITE is HOST TARGET binding site found in the 3′ untranslated region of mRNA encoded by CELSR3, corresponding to a HOST TARGET binding site such as BINDING SITE I, BINDING SITE II or BINDING SITE III. Table 2 illustrates the complementarity of the nucleotide sequences of CELSR3 BINDING SITE, designated SEQ ID:824, to the nucleotide sequence of VGAM162 RNA, herein designated VGAM RNA, also designated SEQ ID:497.

A function of VGAM162 is therefore inhibition of Cadherin, EGF LAG Seven-pass G-type Receptor 3 (flamingo homolog, Drosophila) (CELSR3, Accession NM_{—}001407), a gene which interacts in a homophilic manner in connecting cells. Accordingly, utilities of VGAM162 include diagnosis, prevention and treatment of diseases and clinical conditions associated with CELSR3. The function of CELSR3 has been established by previous studies. The domain that characterizes epidermal growth factor (EGF; 131530) consists of approximately 50 amino acids with 3 disulfide bonds. EGF-like domains are believed to play a critical role in a number of extracellular events, including cell adhesion and receptor-ligand interactions. Proteins with EGF-like domains often consist of more than 1,000 amino acids, have multiple copies of the EGF-like domain, and contain additional domains known to be involved in specific protein-protein interactions. To identify proteins containing EGF-like domains, Nakayama et al. (1998) searched a database of long cDNA sequences randomly selected from a human brain cDNA library for those that encode an EGF-like motif. They identified several partial cDNAs encoding novel proteins with EGF-like domains, such as EGFL1, which they named MEGF2. Nakayama et al. (1998) isolated a rat cDNA containing the complete Megf2 coding sequence. The predicted Megf2 protein has a signal sequence, 8 cadherin motifs (see OMIM Ref. No. 603006), 6 EGF-like domains, 2 laminin G domains (see OMIM Ref. No. 601033), 7 transmembrane domains, and a cytoplasmic proline-rich sequence. Megf2 appears to have a domain structure identical to that of human MEGF3 (OMIM Ref. No. 604265), whose partial cDNA was also isolated by the authors. Northern blot analysis detected Megf2 expression in several regions of rat brain.

Full details of the abovementioned studies are described in the following publications, the disclosure of which are hereby incorporated by reference:
Nakayama, M.; Nakajima, D.; Nagase, T.; Nomura, N.; Seki, N.; Ohara, O.: Identification of high-molecular-weight proteins with multiple EGF-like motifs by motif-trap screening. Genomics 51: 27-34, 1998.; and
Wu, Q.; Maniatis, T.: Large exons encoding multiple ectodomains are a characteristic feature of protocadherin genes. Proc. Nat. Acad. Sci. 97: 3124-3129, 2000.

Further studies establishing the function and utilities of CELSR3 are found in John Hopkins OMIM database record ID 604264, and in sited publications numbered 1672-1673 listed in the bibliography section hereinbelow, which are also hereby incorporated by reference. G Protein-coupled Receptor 85 (GPR85, Accession NM_{—}018970) is another VGAM162 host target gene. GPR85 BINDING SITE is HOST TARGET binding site found in the 3′ untranslated region of mRNA encoded by GPR85, corresponding to a HOST TARGET binding site such as BINDING SITE I, BINDING SITE II or BINDING SITE III. Table 2 illustrates the complementarity of the nucleotide sequences of GPR85 BINDING SITE, designated SEQ ID:1868, to the nucleotide sequence of VGAM162 RNA, herein designated VGAM RNA, also designated SEQ ID:497.

Another function of VGAM162 is therefore inhibition of G Protein-coupled Receptor 85 (GPR85, Accession NM_{—}018970). Accordingly, utilities of VGAM162 include diagnosis, prevention and treatment of diseases and clinical conditions associated with GPR85. Integrin, Alpha V (vitronectin receptor, alpha polypeptide, antigen CD51) (ITGAV, Accession NM_{—}002210) is another VGAM162 host target gene. ITGAV BINDING SITE is HOST TARGET binding site found in the 3′ untranslated region of mRNA encoded by ITGAV, corresponding to a HOST TARGET binding site such as BINDING SITE I, BINDING SITE II or BINDING SITE III. Table 2 illustrates the complementarity of the nucleotide sequences of ITGAV BINDING SITE, designated SEQ ID:908, to the nucleotide sequence of VGAM162 RNA, herein designated VGAM RNA, also designated SEQ ID:497.

Another function of VGAM162 is therefore inhibition of Integrin, Alpha V (vitronectin receptor, alpha polypeptide, antigen CD51) (ITGAV, Accession NM_{—}002210), a gene which is a member of the integrin family of cell-surface proteins. Accordingly, utilities of VGAM162 include diagnosis, prevention and treatment of diseases and clinical conditions associated with ITGAV. The function of ITGAV has been established by previous studies. A major surface antigen family on human leukocytes includes complement receptor type 3 (CR3A; also called integrin alpha-M, Mac1 or Mol), lymphocyte function-associated antigen type 1 (LFA-1; 153370), and p150,95 (Leu M5; 151510). These antigens share a common beta chain (OMIM Ref. No. 116920) of 94 kD, linked noncovalently to 1 of 3 alpha chains distinctive to each. They promote adhesion of granulocytes to each other and to endothelial cell monolayers. The apparent molecular weight of the Mol alpha chain is 155 to 165 kD, that of the LFA1 alpha subunit is 180 kD, and that of the Leu M5 subunit is 130 to 150 kD. Pierce et al. (1986) purified human Mol to homogeneity from normal granulocytes by affinity chromatography and high performance liquid chromatography (HPLC) and determined the N-terminal amino acid sequence of its alpha subunit. The obtained sequence was identical, except for 2 conservative substitutions, to that of the alpha subunit of Mac1 antigen (Springer et al., 1985). Furthermore, Pierce et al. (1986) found that the N-terminal amino acid sequence of the alpha subunit of Mol was homologous to the alpha subunit of IIb/IIIa, a glycoprotein that serves similar adhesive functions on platelets and is deficient or defective in Glanzmann thrombasthenia (OMIM Ref. No. 273800). Patients with a history of recurrent bacterial infections and an inherited deficiency of all 3 leukocyte membrane surface antigens are thought to have reduced or absent synthesis of the common beta subunit of the antigen family; see 116920. Inflammation plays an essential role in the initiation and progression of atherosclerosis. Simon et al. (2000) presented evidence that it also has a role in vascular repair after mechanical arterial injury (i.e., percutaneous transluminal coronary angioplasty, or PTCA). In animal models of vascular injury, leukocytes are recruited as a precursor to intimal thickening. Markers of leukocyte activation, in particular, increased expression of Mac1, which is responsible for firm leukocyte adhesion to platelets and fibrinogen on denuded vessels, predict restenosis after PTCA. To determine whether Mac1-mediated leukocyte recruitment is causally related to neointimal formation, Simon et al. (2000) subjected Mac1 knockout mice to a mechanical carotid artery dilation and complete endothelial denudation. They found that the selective absence of Mac1 impaired transplatelet leukocyte migration into the vessel wall, reducing leukocyte accumulation. Diminished medial leukocyte accumulation was accompanied by markedly reduced neointimal thickening after vascular injury. These data established a role for inflammation in neointimal thickening and suggested that leukocyte recruitment to mechanically injured arteries may prevent restenosis

Full details of the abovementioned studies are described in the following publications, the disclosure of which are hereby incorporated by reference:
Pierce, M. W.; Remold-O'Donnell, E.; Todd, R. F., III; Arnaout, M. A.: N-terminal sequence of human leukocyte glycoprotein Mol: conservation across species and homology to platelet IIb/IIIa. Biochim. Biophys. Acta 874: 368-371, 1986.; and
Simon, D. I.; Chen, Z.; Seifert, P.; Edelman, E. R.; Ballantyne, C. M.; Rogers, C.: Decreased neointimal formation in Mac-1 −/− mice reveals a role for inflammation in vascular repair a.

Further studies establishing the function and utilities of ITGAV are found in John Hopkins OMIM database record ID 193210, and in sited publications numbered 101-102, 759, 760-761, 10 and 2998-2999 listed in the bibliography section hereinbelow, which are also hereby incorporated by reference. Microtubule-associated Protein, RP/EB Family, Member 2 (MAPRE2, Accession NM_{—}014268) is another VGAM162 host target gene. MAPRE2 BINDING SITE is HOST TARGET binding site found in the 3′ untranslated region of mRNA encoded by MAPRE2, corresponding to a HOST TARGET binding site such as BINDING SITE I, BINDING SITE II or BINDING SITE III. Table 2 illustrates the complementarity of the nucleotide sequences of MAPRE2 BINDING SITE, designated SEQ ID: 1487, to the nucleotide sequence of VGAM162 RNA, herein designated VGAM RNA, also designated SEQ ID:497.

Another function of VGAM162 is therefore inhibition of Microtubule-associated Protein, RP/EB Family, Member 2 (MAPRE2, Accession NM_{—}014268), a gene which The functional inactivation of the APC gene product is a key event in colorectal tumorigenesis. Accordingly, utilities of VGAM162 include diagnosis, prevention and treatment of diseases and clinical conditions associated with MAPRE2. The function of MAPRE2 has been established by previous studies. EB1 family proteins (e.g., MAPREL; 603108) interact with cytoplasmic microtubules in interphase cells, with mitotic spindles, and with the adenomatous polyposis coli (APC; 175100) tumor suppressor gene. The functional inactivation of the APC gene product is a key event in colorectal tumorigenesis. By differential mRNA display of resting and activated T cells, followed by 5-prime RACE, Renner et al. (1997) isolated a cDNA encoding MAPRE2, which they termed RP1. The deduced 327-amino acid protein has significant homology with EB1 family proteins. Northern blot analysis detected a 2.6-kb transcript in T cells activated by 2 signals (i.e., cell surface antigen(s) and/or cytokine) and also in lymphocyte tumor cell lines. Immunoprecipitation analysis indicated that RP1 associates with full-length but not C terminus-deleted APC. Renner et al. (1997) concluded that RP1 may be an immediate-early T-cell regulatory gene. Using immunoprecipitation analysis, Juwana et al. (1999) showed that the N terminus of RP1 interacted with monomeric or polymerized tubulin in fibrosarcoma cell lines. Immunofluorescence microscopy demonstrated that RP1 is localized in the plus ends of microtubule networks in the presence or absence of APC. By radiation hybrid and sequence analyses, Su and Qi (2001) mapped the MAPRE2 gene to 18q12. By FISH, Wadle et al. (2001) assigned the gene to 18q21.

Full details of the abovementioned studies are described in the following publications, the disclosure of which are hereby incorporated by reference:
Juwana, J.-P.; Henderikx, P.; Mischo, A.; Wadle, A.; Fadle, N.; Gerlach, K.; Arends, J. W.; Hoogenboom, H.; Pfreundschuh, M.; Renner, C.: EB/RP gene family encodes tubulin binding proteins. Int. J. Cancer 81: 275-284, 1999.; and
Renner, C.; Pfitzenmeier, J.-P.; Gerlach, K.; Held, G.; Ohnesorge, S.; Sahin, U.; Bauer, S.; Pfreundschuh, M.: RP1, a new member of the adenomatous polyposis coli-binding EB1-like gen.

Further studies establishing the function and utilities of MAPRE2 are found in John Hopkins OMIM database record ID 605789, and in sited publications numbered 178-17 and 1937 listed in the bibliography section hereinbelow, which are also hereby incorporated by reference. Paired Box Gene 6 (aniridia, keratitis) (PAX6, Accession NM_{—}001604) is another VGAM162 host target gene. PAX6 BINDING SITE1 and PAX6 BINDING SITE2 are HOST TARGET binding sites found in untranslated regions of mRNA encoded by PAX6, corresponding to HOST TARGET binding sites such as BINDING SITE I, BINDING SITE II or BINDING SITE III. Table 2 illustrates the complementarity of the nucleotide sequences of PAX6 BINDING SITE1 and PAX6 BINDING SITE2, designated SEQ ID:837 and SEQ ID:726 respectively, to the nucleotide sequence of VGAM162 RNA, herein designated VGAM RNA, also designated SEQ ID:497.

Another function of VGAM162 is therefore inhibition of Paired Box Gene 6 (aniridia, keratitis) (PAX6, Accession NM_{—}001604), a gene which involves in oculogenesis. Accordingly, utilities of VGAM162 include diagnosis, prevention and treatment of diseases and clinical conditions associated with PAX6. The function of PAX6 has been established by previous studies. PAX6 is a member of the paired box gene family and encodes a transcriptional regulator involved in oculogenesis, pancreatic, pituitary and central nervous system development. Hanson and Van Heyningen (1995) reviewed the work on PAX6 in man, mouse, and Drosophila. A chronology was provided, beginning with identification of the ‘paired’ gene as a key regulator of segmentation in Drosophila in 1980 to the discovery by Halder et al. (1995) that ectopic expression of Drosophila Pax6 induces ectopic eye development. Wawersik and Maas (2000) reviewed the role of Pax6 and other genes in vertebrate and fly oculogenesis. Animal model experiments lend further support to the function of PAX6. Lyon (1988) suggested that ‘small eye’ (Sey) in the mouse, which is on chromosome 2, may be homologous to aniridia type II (OMIM Ref. No. 106210) inasmuch as there is a region of conserved homology of synteny between human 11p and mouse chromosome 2. This suggestion was corroborated by van der Meer-de Jong et al. (1990) who found through interspecies backcrosses for linkage mapping that the Sey gene lies between Fshb and Cas-1. In the human, AN2 lies between the 2 cognate genes, FSHB and CAT. Glaser et al. (1990) studied the Sey mutation by localizing in an interspecies backcross between Mus musculus/domesticus and Mus spretus, the region on mouse chromosome 2 carrying 9 evolutionarily conserved DNA clones from proximal human 11p. In Dickie's small eye, they found deletion of 3 clones that encompass the aniridia (AN2) and Wilms tumor susceptibility genes in man. Unlike their human counterparts, the heterozygous Dickie's small eye mice do not develop nephroblastomas. The homology of Sey and AN2 was established by the cloning of the AN2 gene in the human and its homolog in the mouse, and the demonstration of mutations in 3 independent Sey alleles (Hill et al., 1991). The mutations would predictably disrupt the function of the gene, which belongs to the Pax multigene family. This family of developmental genes was first described in Drosophila. A Pax gene referred to as Pax6 is identical to the mouse homolog of the candidate aniridia gene. Matsuo et al. (1993) found an internal deletion of about 600 bp in the Pax6 gene in rats homozygous for the small eye mutation. Deletion was due to a single base insertion that generated an abnormal 5-prime donor splice site. They showed that anterior midbrain crest cells in the homozygous embryos reached the eye rudiments but did not migrate any further to the nasal rudiments, suggesting that the Pax6 gene is involved in conducting migration of neural crest cells from the anterior midbrain.

It is appreciated that the abovementioned animal model for PAX6 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Wawersik, S.; Maas, R. L.: Vertebrate eye development as modeled in Drosophila. Hum. Molec. Genet. 9: 917-925, 2000.; and
Glaser, T.; Lane, J.; Housman, D.: A mouse model of the aniridia-Wilms tumor deletion syndrome. Science 250: 823-827, 1990.

Further studies establishing the function and utilities of PAX6 are found in John Hopkins OMIM database record ID 607108, and in sited publications numbered 1271, 2833, 1856, 2834, 2936-2939, 1282-1284, 2686-1286, 2940, 2941, 2942-2943, 1289-1291, 2944, 2945-2946, 1419, 2835, 2947-2949, 1279, 2950, 2951-1249, 2479, 2952, 2955, 2953-125 and 1 listed in the bibliography section hereinbelow, which are also hereby incorporated by reference. Solute Carrier Family 31 (copper transporters), Member 1 (SLC31A1, Accession NM_{—}001859) is another VGAM162 host target gene. SLC31A1 BINDING SITE is HOST TARGET binding site found in the 3′ untranslated region of mRNA encoded by SLC31A1, corresponding to a HOST TARGET binding site such as BINDING SITE I, BINDING SITE II or BINDING SITE III. Table 2 illustrates the complementarity of the nucleotide sequences of SLC31A1 BINDING SITE, designated SEQ ID:864, to the nucleotide sequence of VGAM162 RNA, herein designated VGAM RNA, also designated SEQ ID:497.

Another function of VGAM162 is therefore inhibition of Solute Carrier Family 31 (copper transporters), Member 1 (SLC31A1, Accession NM_{—}001859), a gene which is involved in high-affinity copper uptake. Accordingly, utilities of VGAM162 include diagnosis, prevention and treatment of diseases and clinical conditions associated with SLC31A1. The function of SLC31A1 has been established by previous studies. Moller et al. (2000) found that cells expressing CTR1 but not those expressing CTR2 showed a dramatic hyperaccumulation of radioactive copper, comparable to that seen in fibroblasts from Menkes disease patients. However, in contrast to the Menkes syndrome fibroblasts, the CTR1-expressing fibroblasts had an efflux rate similar to normal fibroblasts. Animal model experiments lend further support to the function of SLC31A1. To test the hypothesis that CTR1 is required for copper delivery to mammalian cells, Kuo et al. (2001) inactivated the Ctrl gene in mice by targeted mutagenesis. They observed early embyronic lethality in homozygous mutant embryos and a deficiency in copper uptake in the brains of heterozygous animals. A study of the spatial and temporal expression pattern of Ctrl during mouse development and adulthood further showed that Ctrl is ubiquitously transcribed with highest expression observed in the specialized epithelia of the choroid plexus and renal tubules and in connective tissues of the eye, ovary, and testis. Similarly, Lee et al. (2001) showed that the mouse Ctrl gene encodes a component of the copper transport machinery and that mice heterozygous for Ctrl exhibit tissue-specific defects in copper accumulation and in the activities of copper-dependent enzymes. Mice completely deficient for Ctrl exhibited profound growth and developmental defects and died in utero in midgestation

It is appreciated that the abovementioned animal model for SLC31A1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Lee, J.; Prohaska, J. R.; Thiele, D. J.: Essential role for mammalian copper transporter Ctrl in copper homeostasis and embryonic development. Proc. Nat. Acad. Sci. 98: 6842-6847, 2001.; and
Moller, L. B.; Petersen, C.; Lund, C.; Horn, N.: Characterization of the hCTR1 gene: genomic organization, functional expression, and identification of a highly homologous processed gen.

Further studies establishing the function and utilities of SLC31A1 are found in John Hopkins OMIM database record ID 603085, and in sited publications numbered 241-247 listed in the bibliography section hereinbelow, which are also hereby incorporated by reference. Solute Carrier Family 4, Sodium Bicarbonate Cotransporter, Member 7 (SLC4A7, Accession NM_{—}003615) is another VGAM162 host target gene. SLC4A7 BINDING SITE is HOST TARGET binding site found in the 3′ untranslated region of mRNA encoded by SLC4A7, corresponding to a HOST TARGET binding site such as BINDING SITE I, BINDING SITE II or BINDING SITE III. Table 2 illustrates the complementarity of the nucleotide sequences of SLC4A7 BINDING SITE, designated SEQ ID: 1043, to the nucleotide sequence of VGAM162 RNA, herein designated VGAM RNA, also designated SEQ ID:497.

Another function of VGAM162 is therefore inhibition of Solute Carrier Family 4, Sodium Bicarbonate Cotransporter, Member 7 (SLC4A7, Accession NM_{—}003615), a gene which mediates the coupled movement of sodium and bicarbonate ions across the plasma membrane. Accordingly, utilities of VGAM162 include diagnosis, prevention and treatment of diseases and clinical conditions associated with SLC4A7. The function of SLC4A7 has been established by previous studies. By searching an EST database for sequences related to the pNBC variant of NBC1 (OMIM Ref. No. SLC4A4), Pushkin et al. (1999) identified ESTs encoding SLC4A7, which they called NBC3. They isolated human muscle cDNAs representing a full-length NBC3 coding sequence. The predicted 1,214-amino acid muscle NBC3 variant, which the authors referred to as mNBC3, contains 12 putative transmembrane domains, with cytoplasmic N and C termini. mNBC3 has 1 putative stilbene-binding motif, numerous potential intracellular phosphorylation sites, potential sites for myristylation and amidation, and potential N-linked glycosylation sites in the exofacial loops between transmembrane domains 1 and 2, and 5 and 6. mNBC3 shares 78% amino acid sequence homology with the NBC2 variant (Ishibashi et al., 1998) of SLC4A7, 46% homology with the kNBC variant of NBC1, 39% homology with the pNBC variant of NBC1, and 29% homology with AE3 (SLC4A3; 106195). Expression of mNBC3 in Xenopus oocytes demonstrated that it is a stilbene-insensitive 5-(N-ethyl-N-isopropyl)-amiloride (EIPA)-inhibitable NBC. The SLC4A7 gene spans approximately 80 kb and contains 25 exons. Northern blot analysis of a number of human tissues detected an approximately 7.8-kb mNBC3 transcript only in skeletal muscle and heart. Burnham et al. (2000) stated that the NBC2 (Ishibashi et al., 1998) and mNBC3 (Pushkin et al., 1999) cDNAs share several kb of identical sequence. Although the deduced protein sequences differ in their N and C termini and each protein contains an interior region not present in the other, the corresponding blocks share more than 99% identity. Thus, Burnham et al. (2000) suggested that NBC2 and mNBC3 are encoded by the same gene. Burnham et al. (2000) isolated a human melanoma cell cDNA that encodes a protein containing elements previously thought to be characteristic of each of the variants NBC2 and mNBC3. Northern blot analysis of several human tissues using a probe specific to NBC2 detected expression mainly in lymph node and brain. Northern blot analysis using a probe specific to mNBC3 showed highest expression in skeletal muscle and heart and lower expression in lymph node, whole brain, adrenal gland, trachea, thyroid, stomach, pancreas, kidney, liver, lung, and placenta. Burnham et al. (2000) concluded that the melanoma cell, NBC2, and mNBC3 cDNAs represent 3 alternate transcripts of the SLC4A7 gene, which they called NBC2

Full details of the abovementioned studies are described in the following publications, the disclosure of which are hereby incorporated by reference:
Pushkin, A.; Abuladze, N.; Lee, I.; Newman, D.; Hwang, J.; Kurtz, I.: Mapping of the human NBC3 (SLC4A7) gene to chromosome 3p22. Genomics 57: 321-322, 1999. Note: Correction: Genomics 58: 216 and 321-322, 1999.; and
Soleimani, M.; Burnham, C. E.: Physiologic and molecular aspects of the Na(+):HCO(3−) cotransporter in health and disease processes. Kidney Int. 57: 371-384, 2000.

Further studies establishing the function and utilities of SLC4A7 are found in John Hopkins OMIM database record ID 603353, and in sited publications numbered 248-25 and 1820 listed in the bibliography section hereinbelow, which are also hereby incorporated by reference. Zinc Finger Protein 22 (KOX 15) (ZNF22, Accession XM_{—}166153) is another VGAM162 host target gene. ZNF22 BINDING SITE is HOST TARGET binding site found in the 3′ untranslated region of mRNA encoded by ZNF22, corresponding to a HOST TARGET binding site such as BINDING SITE I, BINDING SITE II or BINDING SITE III. Table 2 illustrates the complementarity of the nucleotide sequences of ZNF22 BINDING SITE, designated SEQ ID:3527, to the nucleotide sequence of VGAM162 RNA, herein designated VGAM RNA, also designated SEQ ID:497.

Another function of VGAM162 is therefore inhibition of Zinc Finger Protein 22 (KOX 15) (ZNF22, Accession XM_{—}166153). Accordingly, utilities of VGAM162 include diagnosis, prevention and treatment of diseases and clinical conditions associated with ZNF22. ARGBP2 (Accession NM_{—}003603) is another VGAM162 host target gene. ARGBP2 BINDING SITE is HOST TARGET binding site found in the 5′ untranslated region of mRNA encoded by ARGBP2, corresponding to a HOST TARGET binding site such as BINDING SITE I, BINDING SITE II or BINDING SITE III. Table 2 illustrates the complementarity of the nucleotide sequences of ARGBP2 BINDING SITE, designated SEQ ID:1041, to the nucleotide sequence of VGAM162 RNA, herein designated VGAM RNA, also designated SEQ ID:497.

Another function of VGAM162 is therefore inhibition of ARGBP2 (Accession NM_{—}003603). Accordingly, utilities of VGAM162 include diagnosis, prevention and treatment of diseases and clinical conditions associated with ARGBP2. Chromosome 20 Open Reading Frame 7 (C20orf7, Accession NM_{—}024120) is another VGAM162 host target gene. C20orf7 BINDING SITE is HOST TARGET binding site found in the 3′ untranslated region of mRNA encoded by C20orf7, corresponding to a HOST TARGET binding site such as BINDING SITE I, BINDING SITE II or BINDING SITE III. Table 2 illustrates the complementarity of the nucleotide sequences of C20orf7 BINDING SITE, designated SEQ ID:2056, to the nucleotide sequence of VGAM162 RNA, herein designated VGAM RNA, also designated SEQ ID:497.

Another function of VGAM162 is therefore inhibition of Chromosome 20 Open Reading Frame 7 (C20orf7, Accession NM_{—}024120). Accordingly, utilities of VGAM162 include diagnosis, prevention and treatment of diseases and clinical conditions associated with C20orf7. FLJ12704 (Accession NM_{—}024998) is another VGAM162 host target gene. FLJ12704 BINDING SITE is HOST TARGET binding site found in the 3′ untranslated region of mRNA encoded by FLJ12704, corresponding to a HOST TARGET binding site such as BINDING SITE I, BINDING SITE II or BINDING SITE III. Table 2 illustrates the complementarity of the nucleotide sequences of FLJ12704 BINDING SITE, designated SEQ ID:2125, to the nucleotide sequence of VGAM162 RNA, herein designated VGAM RNA, also designated SEQ ID:497.

Another function of VGAM162 is therefore inhibition of FLJ12704 (Accession NM_{—}024998). Accordingly, utilities of VGAM162 include diagnosis, prevention and treatment of diseases and clinical conditions associated with FLJ12704. FLJ13194 (Accession NM_{—}025146) is another VGAM162 host target gene. FLJ13194 BINDING SITE is HOST TARGET binding site found in the 3′ untranslated region of mRNA encoded by FLJ13194, corresponding to a HOST TARGET binding site such as BINDING SITE I, BINDING SITE II or BINDING SITE III. Table 2 illustrates the complementarity of the nucleotide sequences of FLJ13194 BINDING SITE, designated SEQ ID:2141, to the nucleotide sequence of VGAM162 RNA, herein designated VGAM RNA, also designated SEQ ID:497.

Another function of VGAM162 is therefore inhibition of FLJ13194 (Accession NM_{—}025146). Accordingly, utilities of VGAM162 include diagnosis, prevention and treatment of diseases and clinical conditions associated with FLJ13194. FLJ20005 (Accession NM_{—}017617) is another VGAM162 host target gene. FLJ20005 BINDING SITE is HOST TARGET binding site found in the 3′ untranslated region of mRNA encoded by FLJ20005, corresponding to a HOST TARGET binding site such as BINDING SITE I, BINDING SITE II or BINDING SITE III. Table 2 illustrates the complementarity of the nucleotide sequences of FLJ20005 BINDING SITE, designated SEQ ID:1731, to the nucleotide sequence of VGAM162 RNA, herein designated VGAM RNA, also designated SEQ ID:497.

Another function of VGAM162 is therefore inhibition of FLJ20005 (Accession NM_{—}017617). Accordingly, utilities of VGAM162 include diagnosis, prevention and treatment of diseases and clinical conditions associated with FLJ20005. FLJ20417 (Accession NM_{—}017810) is another VGAM162 host target gene. FLJ20417 BINDING SITE is HOST TARGET binding site found in the 5′ untranslated region of mRNA encoded by FLJ20417, corresponding to a HOST TARGET binding site such as BINDING SITE I, BINDING SITE II or BINDING SITE III. Table 2 illustrates the complementarity of the nucleotide sequences of FLJ20417 BINDING SITE, designated SEQ ID:1754, to the nucleotide sequence of VGAM162 RNA, herein designated VGAM RNA, also designated SEQ ID:497.

Another function of VGAM162 is therefore inhibition of FLJ20417 (Accession NM_{—}017810). Accordingly, utilities of VGAM162 include diagnosis, prevention and treatment of diseases and clinical conditions associated with FLJ20417. KIAA0712 (Accession NM_{—}014715) is another VGAM162 host target gene. KIAA0712 BINDING SITE is HOST TARGET binding site found in the 3′ untranslated region of mRNA encoded by KIAA0712, corresponding to a HOST TARGET binding site such as BINDING SITE I, BINDING SITE II or BINDING SITE III. Table 2 illustrates the complementarity of the nucleotide sequences of KIAA0712 BINDING SITE, designated SEQ ID:1529, to the nucleotide sequence of VGAM162 RNA, herein designated VGAM RNA, also designated SEQ ID:497.

Another function of VGAM162 is therefore inhibition of KIAA0712 (Accession NM_{—}014715). Accordingly, utilities of VGAM162 include diagnosis, prevention and treatment of diseases and clinical conditions associated with KIAA0712. KIAA0750 (Accession NM_{—}014632) is another VGAM162 host target gene. KIAA0750 BINDING SITE is HOST TARGET binding site found in the 5′ untranslated region of mRNA encoded by KIAA0750, corresponding to a HOST TARGET binding site such as BINDING SITE I, BINDING SITE II or BINDING SITE III. Table 2 illustrates the complementarity of the nucleotide sequences of KIAA0750 BINDING SITE, designated SEQ ID:1510, to the nucleotide sequence of VGAM162 RNA, herein designated VGAM RNA, also designated SEQ ID:497.

Another function of VGAM162 is therefore inhibition of KIAA0750 (Accession NM_{—}014632). Accordingly, utilities of VGAM162 include diagnosis, prevention and treatment of diseases and clinical conditions associated with KIAA0750. KIAA1128 (Accession XM_{—}043596) is another VGAM162 host target gene. KIAA1128 BINDING SITE is HOST TARGET binding site found in the 3′ untranslated region of mRNA encoded by KIAA1128, corresponding to a HOST TARGET binding site such as BINDING SITE I, BINDING SITE II or BINDING SITE III. Table 2 illustrates the complementarity of the nucleotide sequences of KIAA1128 BINDING SITE, designated SEQ ID:2821, to the nucleotide sequence of VGAM162 RNA, herein designated VGAM RNA, also designated SEQ ID:497.

Another function of VGAM162 is therefore inhibition of KIAA1128 (Accession XM_{—}043596). Accordingly, utilities of VGAM162 include diagnosis, prevention and treatment of diseases and clinical conditions associated with KIAA1128. KIAA1281 (Accession XM_{—}114432) is another VGAM162 host target gene. KIAA1281 BINDING SITE is HOST TARGET binding site found in the 3′ untranslated region of mRNA encoded by KIAA1281, corresponding to a HOST TARGET binding site such as BINDING SITE I, BINDING SITE II or BINDING SITE III. Table 2 illustrates the complementarity of the nucleotide sequences of KIAA1281 BINDING SITE, designated SEQ ID:3461, to the nucleotide sequence of VGAM162 RNA, herein designated VGAM RNA, also designated SEQ ID:497.

Another function of VGAM162 is therefore inhibition of KIAA1281 (Accession XM_{—}114432). Accordingly, utilities of VGAM162 include diagnosis, prevention and treatment of diseases and clinical conditions associated with KIAA1281. KIAA1841 (Accession XM_{—}087056) is another VGAM162 host target gene. KIAA1841 BINDING SITE is HOST TARGET binding site found in the 3′ untranslated region of mRNA encoded by KIAA1841, corresponding to a HOST TARGET binding site such as BINDING SITE I, BINDING SITE II or BINDING SITE III. Table 2 illustrates the complementarity of the nucleotide sequences of KIAA1841 BINDING SITE, designated SEQ ID:3151, to the nucleotide sequence of VGAM162 RNA, herein designated VGAM RNA, also designated SEQ ID:497.

Another function of VGAM162 is therefore inhibition of KIAA1841 (Accession XM_{—}087056). Accordingly, utilities of VGAM162 include diagnosis, prevention and treatment of diseases and clinical conditions associated with KIAA1841. Rab11-FIP2 (Accession NM_{—}014904) is another VGAM162 host target gene. Rab11-FIP2 BINDING SITE is HOST TARGET binding site found in the 3′ untranslated region of mRNA encoded by Rab11-FIP2, corresponding to a HOST TARGET binding site such as BINDING SITE I, BINDING SITE II or BINDING SITE III. Table 2 illustrates the complementarity of the nucleotide sequences of Rab11-FIP2 BINDING SITE, designated SEQ ID:1580, to the nucleotide sequence of VGAM162 RNA, herein designated VGAM RNA, also designated SEQ ID:497.

Another function of VGAM162 is therefore inhibition of Rab11-FIP2 (Accession NM_{—}014904). Accordingly, utilities of VGAM162 include diagnosis, prevention and treatment of diseases and clinical conditions associated with Rab11-FIP2. RABEX5 (Accession NM_{—}014504) is another VGAM162 host target gene. RABEX5 BINDING SITE is HOST TARGET binding site found in the 3′ untranslated region of mRNA encoded by RABEX5, corresponding to a HOST TARGET binding site such as BINDING SITE I, BINDING SITE II or BINDING SITE III. Table 2 illustrates the complementarity of the nucleotide sequences of RABEX5 BINDING SITE, designated SEQ ID:1504, to the nucleotide sequence of VGAM162 RNA, herein designated VGAM RNA, also designated SEQ ID:497.

Another function of VGAM162 is therefore inhibition of RABEX5 (Accession NM_{—}014504). Accordingly, utilities of VGAM162 include diagnosis, prevention and treatment of diseases and clinical conditions associated with RABEX5. Serum/glucocorticoid Regulated Kinase-like (SGKL, Accession NM_{—}013257) is another VGAM162 host target gene. SGKL BINDING SITE is HOST TARGET binding site found in the 3′ untranslated region of mRNA encoded by SGKL, corresponding to a HOST TARGET binding site such as BINDING SITE I, BINDING SITE II or BINDING SITE III. Table 2 illustrates the complementarity of the nucleotide sequences of SGKL BINDING SITE, designated SEQ ID: 1438, to the nucleotide sequence of VGAM162 RNA, herein designated VGAM RNA, also designated SEQ ID:497.

Another function of VGAM162 is therefore inhibition of Serum/glucocorticoid Regulated Kinase-like (SGKL, Accession NM_{—}013257). Accordingly, utilities of VGAM162 include diagnosis, prevention and treatment of diseases and clinical conditions associated with SGKL. Synaptotagmin-like 3 (SYTL3, Accession XM_{—}087804) is another VGAM162 host target gene. SYTL3 BINDING SITE is HOST TARGET binding site found in the 3′ untranslated region of mRNA encoded by SYTL3, corresponding to a HOST TARGET binding site such as BINDING SITE I, BINDING SITE II or BINDING SITE III. Table 2 illustrates the complementarity of the nucleotide sequences of SYTL3 BINDING SITE, designated SEQ ID:3183, to the nucleotide sequence of VGAM162 RNA, herein designated VGAM RNA, also designated SEQ ID:497.

Another function of VGAM162 is therefore inhibition of Synaptotagmin-like 3 (SYTL3, Accession XM_{—}087804). Accordingly, utilities of VGAM162 include diagnosis, prevention and treatment of diseases and clinical conditions associated with SYTL3. TTY7 (Accession NM_{—}031926) is another VGAM162 host target gene. TTY7 BINDING SITE is HOST TARGET binding site found in the 3′ untranslated region of mRNA encoded by TTY7, corresponding to a HOST TARGET binding site such as BINDING SITE I, BINDING SITE II or BINDING SITE III. Table 2 illustrates the complementarity of the nucleotide sequences of TTY7 BINDING SITE, designated SEQ ID:2220, to the nucleotide sequence of VGAM162 RNA, herein designated VGAM RNA, also designated SEQ ID:497.

Another function of VGAM162 is therefore inhibition of TTY7 (Accession NM_{—}031926). Accordingly, utilities of VGAM162 include diagnosis, prevention and treatment of diseases and clinical conditions associated with TTY7. LOC113612 (Accession XM_{—}054492) is another VGAM162 host target gene. LOC113612 BINDING SITE is HOST TARGET binding site found in the 3′ untranslated region of mRNA encoded by LOC113612, corresponding to a HOST TARGET binding site such as BINDING SITE I, BINDING SITE II or BINDING SITE III. Table 2 illustrates the complementarity of the nucleotide sequences of LOC113612 BINDING SITE, designated SEQ ID:2968, to the nucleotide sequence of VGAM162 RNA, herein designated VGAM RNA, also designated SEQ ID:497.

Another function of VGAM162 is therefore inhibition of LOC113612 (Accession XM_{—}054492). Accordingly, utilities of VGAM162 include diagnosis, prevention and treatment of diseases and clinical conditions associated with LOC113612. LOC121457 (Accession XM_{—}058563) is another VGAM162 host target gene. LOC121457 BINDING SITE is HOST TARGET binding site found in the 3′ untranslated region of mRNA encoded by LOC121457, corresponding to a HOST TARGET binding site such as BINDING SITE I, BINDING SITE II or BINDING SITE III. Table 2 illustrates the complementarity of the nucleotide sequences of LOC121457 BINDING SITE, designated SEQ ID:2993, to the nucleotide sequence of VGAM162 RNA, herein designated VGAM RNA, also designated SEQ ID:497.

Another function of VGAM162 is therefore inhibition of LOC121457 (Accession XM_{—}058563). Accordingly, utilities of VGAM162 include diagnosis, prevention and treatment of diseases and clinical conditions associated with LOC121457. LOC133491 (Accession XM_{—}059655) is another VGAM162 host target gene. LOC133491 BINDING SITE is HOST TARGET binding site found in the 3′ untranslated region of mRNA encoded by LOC133491, corresponding to a HOST TARGET binding site such as BINDING SITE I, BINDING SITE II or BINDING SITE III. Table 2 illustrates the complementarity of the nucleotide sequences of LOC133491 BINDING SITE, designated SEQ ID:3015, to the nucleotide sequence of VGAM162 RNA, herein designated VGAM RNA, also designated SEQ ID:497.

Another function of VGAM162 is therefore inhibition of LOC133491 (Accession XM_{—}059655). Accordingly, utilities of VGAM162 include diagnosis, prevention and treatment of diseases and clinical conditions associated with LOC133491. LOC143282 (Accession XM_{—}084481) is another VGAM162 host target gene. LOC143282 BINDING SITE is HOST TARGET binding site found in the 3′ untranslated region of mRNA encoded by LOC143282, corresponding to a HOST TARGET binding site such as BINDING SITE I, BINDING SITE II or BINDING SITE III. Table 2 illustrates the complementarity of the nucleotide sequences of LOC143282 BINDING SITE, designated SEQ ID:3054, to the nucleotide sequence of VGAM162 RNA, herein designated VGAM RNA, also designated SEQ ID:497.

Another function of VGAM162 is therefore inhibition of LOC143282 (Accession XM_{—}084481). Accordingly, utilities of VGAM162 include diagnosis, prevention and treatment of diseases and clinical conditions associated with LOC143282. LOC143524 (Accession XM_{—}084559) is another VGAM162 host target gene. LOC143524 BINDING SITE is HOST TARGET binding site found in the 3′ untranslated region of mRNA encoded by LOC143524, corresponding to a HOST TARGET binding site such as BINDING SITE I, BINDING SITE II or BINDING SITE III. Table 2 illustrates the complementarity of the nucleotide sequences of LOC143524 BINDING SITE, designated SEQ ID:3059, to the nucleotide sequence of VGAM162 RNA, herein designated VGAM RNA, also designated SEQ ID:497.

Another function of VGAM162 is therefore inhibition of LOC143524 (Accession XM_{—}084559). Accordingly, utilities of VGAM162 include diagnosis, prevention and treatment of diseases and clinical conditions associated with LOC143524. LOC144559 (Accession XM_{—}084896) is another VGAM162 host target gene. LOC144559 BINDING SITE is HOST TARGET binding site found in the 5′ untranslated region of mRNA encoded by LOC144559, corresponding to a HOST TARGET binding site such as BINDING SITE I, BINDING SITE II or BINDING SITE III. Table 2 illustrates the complementarity of the nucleotide sequences of LOC144559 BINDING SITE, designated SEQ ID:3066, to the nucleotide sequence of VGAM162 RNA, herein designated VGAM RNA, also designated SEQ ID:497.

Another function of VGAM162 is therefore inhibition of LOC144559 (Accession XM_{—}084896). Accordingly, utilities of VGAM162 include diagnosis, prevention and treatment of diseases and clinical conditions associated with LOC144559. LOC153222 (Accession XM_{—}087631) is another VGAM162 host target gene. LOC153222 BINDING SITE is HOST TARGET binding site found in the 3′ untranslated region of mRNA encoded by LOC153222, corresponding to a HOST TARGET binding site such as BINDING SITE I, BINDING SITE II or BINDING SITE III. Table 2 illustrates the complementarity of the nucleotide sequences of LOC153222 BINDING SITE, designated SEQ ID:3180, to the nucleotide sequence of VGAM162 RNA, herein designated VGAM RNA, also designated SEQ ID:497.

Another function of VGAM162 is therefore inhibition of LOC153222 (Accession XM_{—}087631). Accordingly, utilities of VGAM162 include diagnosis, prevention and treatment of diseases and clinical conditions associated with LOC153222. LOC219623 (Accession XM_{—}166143) is another VGAM162 host target gene. LOC219623 BINDING SITE is HOST TARGET binding site found in the 5′ untranslated region of mRNA encoded by LOC219623, corresponding to a HOST TARGET binding site such as BINDING SITE I, BINDING SITE II or BINDING SITE III. Table 2 illustrates the complementarity of the nucleotide sequences of LOC219623 BINDING SITE, designated SEQ ID:3525, to the nucleotide sequence of VGAM162 RNA, herein designated VGAM RNA, also designated SEQ ID:497.

Another function of VGAM162 is therefore inhibition of LOC219623 (Accession XM_{—}166143). Accordingly, utilities of VGAM162 include diagnosis, prevention and treatment of diseases and clinical conditions associated with LOC219623. LOC219918 (Accession XM_{—}166197) is another VGAM162 host target gene. LOC219918 BINDING SITE is HOST TARGET binding site found in the 5′ untranslated region of mRNA encoded by LOC219918, corresponding to a HOST TARGET binding site such as BINDING SITE I, BINDING SITE II or BINDING SITE III. Table 2 illustrates the complementarity of the nucleotide sequences of LOC219918 BINDING SITE, designated SEQ ID:3530, to the nucleotide sequence of VGAM162 RNA, herein designated VGAM RNA, also designated SEQ ID:497.

Another function of VGAM162 is therefore inhibition of LOC219918 (Accession XM_{—}166197). Accordingly, utilities of VGAM162 include diagnosis, prevention and treatment of diseases and clinical conditions associated with LOC219918. LOC220071 (Accession XM_{—}167848) is another VGAM162 host target gene. LOC220071 BINDING SITE is HOST TARGET binding site found in the 5′ untranslated region of mRNA encoded by LOC220071, corresponding to a HOST TARGET binding site such as BINDING SITE I, BINDING SITE II or BINDING SITE III. Table 2 illustrates the complementarity of the nucleotide sequences of LOC220071 BINDING SITE, designated SEQ ID:3602, to the nucleotide sequence of VGAM162 RNA, herein designated VGAM RNA, also designated SEQ ID:497.

Another function of VGAM162 is therefore inhibition of LOC220071 (Accession XM_{—}167848). Accordingly, utilities of VGAM162 include diagnosis, prevention and treatment of diseases and clinical conditions associated with LOC220071. LOC254431 (Accession XM_{—}173024) is another VGAM162 host target gene. LOC254431 BINDING SITE is HOST TARGET binding site found in the 3′ untranslated region of mRNA encoded by LOC254431, corresponding to a HOST TARGET binding site such as BINDING SITE I, BINDING SITE II or BINDING SITE III. Table 2 illustrates the complementarity of the nucleotide sequences of LOC254431 BINDING SITE, designated SEQ ID:3717, to the nucleotide sequence of VGAM162 RNA, herein designated VGAM RNA, also designated SEQ ID:497.

Another function of VGAM162 is therefore inhibition of LOC254431 (Accession XM_{—}173024). Accordingly, utilities of VGAM162 include diagnosis, prevention and treatment of diseases and clinical conditions associated with LOC254431. LOC255515 (Accession XM_{—}171185) is another VGAM162 host target gene. LOC255515 BINDING SITE is HOST TARGET binding site found in the 3′ untranslated region of mRNA encoded by LOC255515, corresponding to a HOST TARGET binding site such as BINDING SITE I, BINDING SITE II or BINDING SITE III. Table 2 illustrates the complementarity of the nucleotide sequences of LOC255515 BINDING SITE, designated SEQ ID:3688, to the nucleotide sequence of VGAM162 RNA, herein designated VGAM RNA, also designated SEQ ID:497.

Another function of VGAM162 is therefore inhibition of LOC255515 (Accession XM_{—}171185). Accordingly, utilities of VGAM162 include diagnosis, prevention and treatment of diseases and clinical conditions associated with LOC255515. LOC56959 (Accession XM_{—}088578) is another VGAM162 host target gene. LOC56959 BINDING SITE is HOST TARGET binding site found in the 5′ untranslated region of mRNA encoded by LOC56959, corresponding to a HOST TARGET binding site such as BINDING SITE I, BINDING SITE II or BINDING SITE III. Table 2 illustrates the complementarity of the nucleotide sequences of LOC56959 BINDING SITE, designated SEQ ID:3219, to the nucleotide sequence of VGAM162 RNA, herein designated VGAM RNA, also designated SEQ ID:497.

Another function of VGAM162 is therefore inhibition of LOC56959 (Accession XM_{—}088578). Accordingly, utilities of VGAM162 include diagnosis, prevention and treatment of diseases and clinical conditions associated with LOC56959. LOC90906 (Accession XM_{—}034809) is another VGAM162 host target gene. LOC90906 BINDING SITE is HOST TARGET binding site found in the 3′ untranslated region of mRNA encoded by LOC90906, corresponding to a HOST TARGET binding site such as BINDING SITE I, BINDING SITE II or BINDING SITE III. Table 2 illustrates the complementarity of the nucleotide sequences of LOC90906 BINDING SITE, designated SEQ ID:2685, to the nucleotide sequence of VGAM162 RNA, herein designated VGAM RNA, also designated SEQ ID:497.

Another function of VGAM162 is therefore inhibition of LOC90906 (Accession XM_{—}034809). Accordingly, utilities of VGAM162 include diagnosis, prevention and treatment of diseases and clinical conditions associated with LOC90906. FIG. 1 further provides a conceptual description of a novel bioinformatically detected viral gene of the present invention, referred to here as Viral Genomic Address Messenger 163 (VGAM163) viral gene, which modulates expression of respective host target genes thereof, the function and utility of which host target genes is known in the art.

VGAM163 is a novel bioinformatically detected regulatory, non protein coding, viral micro RNA (miRNA) gene. The method by which VGAM163 was detected is described hereinabove with reference to FIGS. 1-8.

VGAM163 gene, herein designated VGAM GENE, is a viral gene contained in the genome of Vaccinia Virus. VGAM163 host target gene, herein designated VGAM HOST TARGET GENE, is a human gene contained in the human genome.

VGAM163 gene encodes a VGAM163 precursor RNA, herein designated VGAM PRECURSOR RNA. Similar to other miRNA genes, and unlike most ordinary genes, VGAM163 precursor RNA does not encode a protein. A nucleotide sequence identical or highly similar to the nucleotide sequence of VGAM163 precursor RNA is designated SEQ ID:149, and is provided hereinbelow with reference to the sequence listing part. Nucleotide sequence SEQ ID: 149 is located at position 83968 relative to the genome of Vaccinia Virus.

VGAM163 precursor RNA folds onto itself, forming VGAM163 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM163 folded precursor RNA into VGAM163 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM163 RNA is designated SEQ ID:498, and is provided hereinbelow with reference to the sequence listing part.

VGAM163 host target gene, herein designated VGAM HOST TARGET GENE, encodes a corresponding messenger RNA, VGAM163 host target RNA, herein designated VGAM HOST TARGET RNA. VGAM163 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM163 RNA, herein designated VGAM RNA, binds complementarily to one or more host target binding sites located in untranslated regions of VGAM163 host target RNA, herein designated VGAM HOST TARGET RNA. This complementary binding is due to the fact that the nucleotide sequence of VGAM163 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM163 RNA, herein designated VGAM RNA, may have a different number of host target binding sites in untranslated regions of a VGAM163 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM163 RNA, herein designated VGAM RNA, to host target binding sites on VGAM163 host target RNA, herein designated VGAM HOST TARGET RNA, such as BINDING SITE I, BINDING SITE II and BINDING SITE III, inhibits translation of VGAM163 host target RNA into VGAM163 host target protein, herein designated VGAM HOST TARGET PROTEIN. VGAM host target protein is therefore outlined by a broken line.

It is appreciated that VGAM163 host target gene, herein designated VGAM HOST TARGET GENE, in fact represents a plurality of VGAM163 host target genes. The mRNA of each one of this plurality of VGAM163 host target genes comprises one or more host target binding sites, each having a nucleotide sequence which is at least partly complementary to VGAM163 RNA, herein designated VGAM RNA, and which when bound by VGAM163 RNA causes inhibition of translation of respective one or more VGAM163 host target proteins.

It is further appreciated by one skilled in the art that the mode of translational inhibition illustrated by FIG. 1 with specific reference to translational inhibition exerted by VGAM163 gene, herein designated VGAM GENE, on one or more VGAM163 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM163 is inhibition of expression of host target genes, as part of a novel viral mechanism of attacking a host. Accordingly, utilities of VGAM163 include diagnosis, prevention and treatment of viral infection by Vaccinia Virus. Specific functions, and accordingly utilities, of VGAM163 correlate with, and may be deduced from, the identity of the host target genes which VGAM163 binds and inhibits, and the function of these host target genes, as elaborated hereinbelow.

Nucleotide sequences of the VGAM163 precursor RNA, herein designated VGAM PRECURSOR RNA, and of the ‘diced’ VGAM163 RNA, herein designated VGAM RNA, and a schematic representation of the secondary folding of VGAM163 folded precursor RNA, herein designated VGAM FOLDED PRECURSOR RNA, of VGAM163 are further described hereinbelow with reference to Table 1.

Nucleotide sequences of host target binding sites, such as BINDING SITE-I, BINDING SITE-II and BINDING SITE-III of FIG. 1, found on VGAM163 host target RNA, and schematic representation of the complementarity of each of these host target binding sites to VGAM163 RNA, herein designated VGAM RNA, are described hereinbelow with reference to Table 2.

As mentioned hereinabove with reference to FIG. 1, a function of VGAM163 gene, herein designated VGAM is inhibition of expression of VGAM163 target genes. It is appreciated that specific functions, and accordingly utilities, of VGAM163 correlate with, and may be deduced from, the identity of the target genes which VGAM163 binds and inhibits, and the function of these target genes, as elaborated hereinbelow.

Enamelin (ENAM, Accession NM_{—}031889) is a VGAM163 host target gene. ENAM BINDING SITE is HOST TARGET binding site found in the 5′ untranslated region of mRNA encoded by ENAM, corresponding to a HOST TARGET binding site such as BINDING SITE I, BINDING SITE II or BINDING SITE III. Table 2 illustrates the complementarity of the nucleotide sequences of ENAM BINDING SITE, designated SEQ ID:2216, to the nucleotide sequence of VGAM163 RNA, herein designated VGAM RNA, also designated SEQ ID:498.

A function of VGAM163 is therefore inhibition of Enamelin (ENAM, Accession NM_{—}031889). Accordingly, utilities of VGAM163 include diagnosis, prevention and treatment of diseases and clinical conditions associated with ENAM. Ephrin-A5 (EFNA5, Accession NM_{—}001962) is another VGAM163 host target gene. EFNA5 BINDING SITE is HOST TARGET binding site found in the 3′ untranslated region of mRNA encoded by EFNA5, corresponding to a HOST TARGET binding site such as BINDING SITE I, BINDING SITE II or BINDING SITE III. Table 2 illustrates the complementarity of the nucleotide sequences of EFNA5 BINDING SITE, designated SEQ ID:876, to the nucleotide sequence of VGAM163 RNA, herein designated VGAM RNA, also designated SEQ ID:498.

Another function of VGAM163 is therefore inhibition of Ephrin-A5 (EFNA5, Accession NM_{—}001962). Accordingly, utilities of VGAM163 include diagnosis, prevention and treatment of diseases and clinical conditions associated with EFNA5. LOC159199 (Accession XM_{—}089441) is another VGAM163 host target gene. LOC159199 BINDING SITE is HOST TARGET binding site found in the 5′ untranslated region of mRNA encoded by LOC159199, corresponding to a HOST TARGET binding site such as BINDING SITE I, BINDING SITE II or BINDING SITE III. Table 2 illustrates the complementarity of the nucleotide sequences of LOC159199 BINDING SITE, designated SEQ ID:3236, to the nucleotide sequence of VGAM163 RNA, herein designated VGAM RNA, also designated SEQ ID:498.

Another function of VGAM163 is therefore inhibition of LOC159199 (Accession XM_{—}089441). Accordingly, utilities of VGAM163 include diagnosis, prevention and treatment of diseases and clinical conditions associated with LOC159199. LOC90321 (Accession XM_{—}030896) is another VGAM163 host target gene. LOC90321 BINDING SITE is HOST TARGET binding site found in the 3′ untranslated region of mRNA encoded by LOC90321, corresponding to a HOST TARGET binding site such as BINDING SITE I, BINDING SITE II or BINDING SITE III. Table 2 illustrates the complementarity of the nucleotide sequences of LOC90321 BINDING SITE, designated SEQ ID:2630, to the nucleotide sequence of VGAM163 RNA, herein designated VGAM RNA, also designated SEQ ID:498.

Another function of VGAM163 is therefore inhibition of LOC90321 (Accession XM_{—}030896). Accordingly, utilities of VGAM163 include diagnosis, prevention and treatment of diseases and clinical conditions associated with LOC90321. FIG. 1 further provides a conceptual description of a novel bioinformatically detected viral gene of the present invention, referred to here as Viral Genomic Address Messenger 164 (VGAM164) viral gene, which modulates expression of respective host target genes thereof, the function and utility of which host target genes is known in the art.

VGAM164 is a novel bioinformatically detected regulatory, non protein coding, viral micro RNA (miRNA) gene. The method by which VGAM164 was detected is described hereinabove with reference to FIGS. 1-8.

VGAM164 gene, herein designated VGAM GENE, is a viral gene contained in the genome of Vaccinia Virus. VGAM164 host target gene, herein designated VGAM HOST TARGET GENE, is a human gene contained in the human genome.

VGAM164 gene encodes a VGAM164 precursor RNA, herein designated VGAM PRECURSOR RNA. Similar to other miRNA genes, and unlike most ordinary genes, VGAM164 precursor RNA does not encode a protein. A nucleotide sequence identical or highly similar to the nucleotide sequence of VGAM164 precursor RNA is designated SEQ ID:150, and is provided hereinbelow with reference to the sequence listing part. Nucleotide sequence SEQ ID:150 is located at position 84375 relative to the genome of Vaccinia Virus.

VGAM164 precursor RNA folds onto itself, forming VGAM164 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM164 folded precursor RNA into VGAM164 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM164 RNA is designated SEQ ID:499, and is provided hereinbelow with reference to the sequence listing part.

VGAM164 host target gene, herein designated VGAM HOST TARGET GENE, encodes a corresponding messenger RNA, VGAM164 host target RNA, herein designated VGAM HOST TARGET RNA. VGAM164 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM164 RNA, herein designated VGAM RNA, binds complementarily to one or more host target binding sites located in untranslated regions of VGAM164 host target RNA, herein designated VGAM HOST TARGET RNA. This complementary binding is due to the fact that the nucleotide sequence of VGAM164 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM164 RNA, herein designated VGAM RNA, may have a different number of host target binding sites in untranslated regions of a VGAM164 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM164 RNA, herein designated VGAM RNA, to host target binding sites on VGAM164 host target RNA, herein designated VGAM HOST TARGET RNA, such as BINDING SITE I, BINDING SITE II and BINDING SITE III, inhibits translation of VGAM164 host target RNA into VGAM164 host target protein, herein designated VGAM HOST TARGET PROTEIN. VGAM host target protein is therefore outlined by a broken line.

It is appreciated that VGAM164 host target gene, herein designated VGAM HOST TARGET GENE, in fact represents a plurality of VGAM164 host target genes. The mRNA of each one of this plurality of VGAM164 host target genes comprises one or more host target binding sites, each having a nucleotide sequence which is at least partly complementary to VGAM164 RNA, herein designated VGAM RNA, and which when bound by VGAM164 RNA causes inhibition of translation of respective one or more VGAM164 host target proteins.

It is further appreciated by one skilled in the art that the mode of translational inhibition illustrated by FIG. 1 with specific reference to translational inhibition exerted by VGAM164 gene, herein designated VGAM GENE, on one or more VGAM164 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM164 is inhibition of expression of host target genes, as part of a novel viral mechanism of attacking a host. Accordingly, utilities of VGAM164 include diagnosis, prevention and treatment of viral infection by Vaccinia Virus. Specific functions, and accordingly utilities, of VGAM164 correlate with, and may be deduced from, the identity of the host target genes which VGAM164 binds and inhibits, and the function of these host target genes, as elaborated hereinbelow.

Nucleotide sequences of the VGAM164 precursor RNA, herein designated VGAM PRECURSOR RNA, and of the ‘diced’ VGAM164 RNA, herein designated VGAM RNA, and a schematic representation of the secondary folding of VGAM164 folded precursor RNA, herein designated VGAM FOLDED PRECURSOR RNA, of VGAM164 are further described hereinbelow with reference to Table 1.

Nucleotide sequences of host target binding sites, such as BINDING SITE-I, BINDING SITE-II and BINDING SITE-III of FIG. 1, found on VGAM164 host target RNA, and schematic representation of the complementarity of each of these host target binding sites to VGAM164 RNA, herein designated VGAM RNA, are described hereinbelow with reference to Table 2.

As mentioned hereinabove with reference to FIG. 1, a function of VGAM164 gene, herein designated VGAM is inhibition of expression of VGAM164 target genes. It is appreciated that specific functions, and accordingly utilities, of VGAM164 correlate with, and may be deduced from, the identity of the target genes which VGAM164 binds and inhibits, and the function of these target genes, as elaborated hereinbelow.

V-Ki-ras2 Kirsten Rat Sarcoma 2 Viral Oncogene Homolog (KRAS2, Accession NM_{—}004985) is a VGAM164 host target gene. KRAS2 BINDING SITE1 and KRAS2 BINDING SITE2 are HOST TARGET binding sites found in untranslated regions of mRNA encoded by KRAS2, corresponding to HOST TARGET binding sites such as BINDING SITE I, BINDING SITE II or BINDING SITE III. Table 2 illustrates the complementarity of the nucleotide sequences of KRAS2 BINDING SITE1 and KRAS2 BINDING SITE2, designated SEQ ID:1166 and SEQ ID:2331 respectively, to the nucleotide sequence of VGAM164 RNA, herein designated VGAM RNA, also designated SEQ ID:499.

A function of VGAM164 is therefore inhibition of V-Ki-ras2 Kirsten Rat Sarcoma 2 Viral Oncogene Homolog (KRAS2, Accession NM_{—}004985), a gene which ras proteins bind gdp/gtp and possess intrinsic gtpase activity. Accordingly, utilities of VGAM164 include diagnosis, prevention and treatment of diseases and clinical conditions associated with KRAS2. The function of KRAS2 has been established by previous studies. See 190020. KRAS2 is on chromosome 12; KRAS1P, a pseudogene, is on chromosome 6. Weinberg (1982) suggested that the then-recognized cellular oncogenes could be assigned to a small number of gene families; e.g., the ras family with at least 4 distinct oncogenes, and the src-yes-mos family with another 3. The probable role of at least 2 oncogenes in normal differentiation is indicated by the findings of transcription of KRAS and the McDonough strain of feline sarcoma virus (FMS) during mouse development (Muller et al., 1983). Furthermore, the differences in transcription in different tissues suggested a specific role for each: FMS was expressed in extraembryonic structures or in transport in these tissues, whereas KRAS was expressed ubiquitously. KRAS, with a length of more than 30 kb, is much larger than HRAS (OMIM Ref. No. 190020) or NRAS (OMIM Ref. No. 164790). Although the 3 ras genes, HRAS, KRAS, and NRAS, have different genetic structures, all code for proteins of 189 amino acid residues, generically designated p21. These genes acquire malignant properties by single point mutations that affect the incorporation of the 12th or 61st amino acid residue of their respective p21. KRAS is involved in malignancy much more often than is HRAS. In a study of 96 human tumors or tumor cell lines in the NIH 3T3 transforming system, Pulciani et al. (1982) found a mutated HRAS locus only in T24 bladder cancer cells, whereas transforming KRAS genes were identified in 8 different carcinomas and sarcomas. Holland et al. (2000) transferred, in a tissue-specific manner, genes encoding activated forms of Ras and Akt (OMIM Ref. No. 164730) to astrocytes and neural progenitors in mice. Holland et al. (2000) found that although neither activated Ras nor Akt alone was sufficient to induce glioblastoma multiforme (GBM; 137800) formation, the combination of activated Ras and Akt induced high-grade gliomas with the histologic features of human GBMs. These tumors appeared to arise after gene transfer to neural progenitors, but not after transfer to differentiated astrocytes. Increased activity of RAS is found in many human GBMs, and Holland et al. (2000) demonstrated that Akt activity is increased in most of these tumors, implying that combined activation of these 2 pathways accurately models the biology of this disease. Bezieau et al. (2001) used ARMS (allele-specific amplification method) to evaluate the incidence of NRAS- and KRAS2-activating mutations in patients with multiple myeloma and related disorders. Mutations were more frequent in KRAS2 than in NRAS. The authors concluded that early mutations in these 2 oncogenes may play a major role in the oncogenesis of multiple myeloma and primary plasma cell leukemia. Rajagopalan et al. (2002) systematically evaluated mutation in BRAF (OMIM Ref. No. 164757) and KRAS in 330 colorectal tumors. There were 32 mutations in BRAF, 28 with a V599E mutation (164757.0001) and 1 each with the R4611 (164757.0002), 1462S (164757.0003), G463E (164757.0004), or K600E (164757.0005) mutations. All but 2 mutations seemed to be heterozygous, and in all 20 cases for which normal tissue was available, the mutations were shown to be somatic. In the same set of tumors there were 169 mutations in KRAS. No tumor exhibited mutations in both BRAF and KRAS. There was also a striking difference in the frequency of BRAF mutations between cancers with and without mismatch repair deficiency. All but 1 of the 15 BRAF mutations identified in mismatch repair deficient cases resulted in a V599E substitution. Rajagopalan et al. (2002) concluded their results provide strong support for the hypothesis that BRAF and KRAS mutations are equivalent in their tumorigenic effects. Both genes seem to be mutated at a similar phase of tumorigenesis, after initiation but before malignant conversion. Moreover, no tumor concurrently contained both BRAF and KRAS mutations. Animal model experiments lend further support to the function of KRAS2. Costa et al. (2002) crossed Nf1 heterozygote mice with mice heterozygous for a null mutation in the Kras gene and tested the Nf1 descendants. They found that the double heterozygotes with decreased Ras function had improved learning relative to Nf1 heterozygote mice. Costa et al. (2002) also showed that the Nf1 +/−mice have increased GABA-mediated inhibition and specific deficits in long-term potentiation, both of which can be reversed by decreasing Ras function. Costa et al. (2002) concluded that learning deficits associated with Nf1 may be caused by excessive Ras activity, which leads to impairments in long-term potentiation caused by increased GABA-mediated inhibition.

It is appreciated that the abovementioned animal model for KRAS2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Costa, R. M.; Federov, N. B.; Kogan, J. H.; Murphy, G. G.; Stern, J.; Ohno, M.; Kucherlapati, R.; Jacks, T.; Silva, A. J.: Mechanism for the learning deficits in a mouse model of neurofibromatosis type 1. Nature 415: 526-530, 2002.; and
Rajagopalan, H.; Bardelli, A.; Lengauer, C.; Kinzler, K. W.; Vogelstein, B.; Velculescu, V. E.: RAF/RAS oncogenes and mismatch-repair status. (Letter) Nature 418: 934 only, 2002.

Further studies establishing the function and utilities of KRAS2 are found in John Hopkins OMIM database record ID 190070, and in sited publications numbered 2350-2352, 512, 2353-2355, 697, 2356-2360, 1059-334, 1245, 1358-1374, 513, 1375-1376, 260 and 1378-1392 listed in the bibliography section hereinbelow, which are also hereby incorporated by reference. Astrotactin (ASTN, Accession XM_{—}045113) is another VGAM165 host target gene. ASTN BINDING SITE is HOST TARGET binding site found in the 3′ untranslated region of mRNA encoded by ASTN, corresponding to a HOST TARGET binding site such as BINDING SITE I, BINDING SITE II or BINDING SITE III. Table 2 illustrates the complementarity of the nucleotide sequences of ASTN BINDING SITE, designated SEQ ID:2852, to the nucleotide sequence of VGAM165 RNA, herein designated VGAM RNA, also designated SEQ ID:500.

Another function of VGAM165 is therefore inhibition of Astrotactin (ASTN, Accession XM_{—}045113). Accordingly, utilities of VGAM165 include diagnosis, prevention and treatment of diseases and clinical conditions associated with ASTN. Galactokinase 2 (GALK2, Accession NM_{—}002044) is another VGAM165 host target gene. GALK2 BINDING SITE is HOST TARGET binding site found in the 3′ untranslated region of mRNA encoded by GALK2, corresponding to a HOST TARGET binding site such as BINDING SITE I, BINDING SITE II or BINDING SITE III. Table 2 illustrates the complementarity of the nucleotide sequences of GALK2 BINDING SITE, designated SEQ ID:891, to the nucleotide sequence of VGAM165 RNA, herein designated VGAM RNA, also designated SEQ ID:500.

Another function of VGAM165 is therefore inhibition of Galactokinase 2 (GALK2, Accession NM_{—}002044), a gene which is involved in the metabolism of galactose. Accordingly, utilities of VGAM165 include diagnosis, prevention and treatment of diseases and clinical conditions associated with GALK2. The function of GALK2 has been established by previous studies. Lee et al. (1992) sought to clone a human galactokinase gene by its ability to substitute for the enzyme in yeast. They used a method for identifying mammalian cDNAs by complementation or other functional activity in yeast. They unexpectedly found that the gene which complemented a galactokinase-deficient strain of Saccharomyces cerevisiae mapped not to chromosome 17 (OMIM Ref. No. 604313) but to chromosome 15, thus calling into question which of the genes, GK1 on 17 or GK2 on 15, is the site of the mutation in the galactokinase deficiency form of galactosemia (see OMIM Ref. No. 230200). The strategy they used might be adopted for cloning various human disease genes affecting intermediary metabolism for which yeast mutants are known. (The mapping to chromosome 15 was done by analysis of genomic DNA from a panel of human-rodent somatic cell hybrids using PCR.) Pastuszak et al. (1996) isolated an N-acetylgalactosamine (OMIM Ref. No. GalNAc) kinase from pig kidney and found that partial peptide sequences of this porcine enzyme were 90% similar to human GK2. Enzyme assays showed that human GK2 is a highly efficient GalNAc kinase with galactokinase activity when this sugar is present at high concentrations. Thus, Pastuszak et al. (1996) stated that although human GK2 was identified based on its galactokinase activity, it is actually a GalNAc kinase.

Full details of the abovementioned studies are described in the following publications, the disclosure of which are hereby incorporated by reference:
Lee, R. T.; Peterson, C. L.; Calman, A. F.; Herskowitz, I.; O'Donnell, J. J.: Cloning of a human galactokinase gene (GK2) on chromosome 15 by complementation in yeast. Proc. Nat. Acad. Sci. 89: 10887-10891, 1992.; and
Pastuszak, I.; O'Donnell, J.; Elbein, A. D.: Identification of the GalNAc kinase amino acid sequence. J. Biol. Chem. 271: 23653-23656, 1996.

Further studies establishing the function and utilities of GALK2 are found in John Hopkins OMIM database record ID 137028, and in sited publications numbered 882-883 listed in the bibliography section hereinbelow, which are also hereby incorporated by reference. Myeloid Cell Leukemia Sequence 1 (BCL2-related) (MCL1, Accession NM_{—}021960) is another VGAM165 host target gene. MCL1 BINDING SITE is HOST TARGET binding site found in the 3′ untranslated region of mRNA encoded by MCL1, corresponding to a HOST TARGET binding site such as BINDING SITE I, BINDING SITE II or BINDING SITE III. Table 2 illustrates the complementarity of the nucleotide sequences of MCL1 BINDING SITE, designated SEQ ID:1970, to the nucleotide sequence of VGAM165 RNA, herein designated VGAM RNA, also designated SEQ ID:500.

Another function of VGAM165 is therefore inhibition of Myeloid Cell Leukemia Sequence 1 (BCL2-related) (MCL1, Accession NM_{—}021960), a gene which involved in programming of differentiation and concomitant maintenance of viability. Accordingly, utilities of VGAM165 include diagnosis, prevention and treatment of diseases and clinical conditions associated with MCL1. The function of MCL1 has been established by previous studies. Kozopas et al. (1993) isolated a gene, MCL1, from the ML-1 human myeloid leukemia cell line. Expression of MCL1 increased early in the induction, or programming, of differentiation in ML-1 (at 1-3 hr), before the appearance of differentiation markers and mature morphology (at 1-3 days). MCL1 showed sequence similarity, particularly in the carboxyl portion, to BCL2 (OMIM Ref. No. 151430), a gene involved in normal lymphoid development and in lymphomas with the t(14;18) chromosome translocation. Further, in contrast to proliferation-associated oncogenes, the expression of MCL1 and BCL2 relates to the programming of differentiation/development and cell viability/death. Kozopas et al. (1993) suggested that MCL1 and BCL2 are 2 members of a ‘new’ gene family. Bae et al. (2000) identified a short splicing variant of MCL1, which they termed MCL1S. Sequence analysis indicated that the 271-amino acid variant lacks BCL2 homology domains 1 and 2 and the transmembrane domain due to the splicing out of exon 2 during mRNA processing. Unlike the full-length 350-amino acid MCL1 protein (MCL1L), yeast 2-hybrid analysis showed that MCL1S does not interact with proapoptotic BCL2 family proteins but dimerizes with the antiapoptotic MCL1L. Overexpression of MCL1S induced apoptosis in transfected CHO cells that could be antagonized by a caspase inhibitor or specifically by MCL1L. Therefore, the authors concluded that the fate of MCL1-expressing cells may be regulated through alternative splicing mechanisms and the interactions of the resulting gene products. Using the methods of somatic cell hybrid analysis and fluorescence in situ hybridization, Craig et al. (1994) mapped MCL1 to 1q21. In the mouse, MCL1-related sequences were mapped to positions on 2 mouse chromosomes, 3 and 5, using haplotype analysis of an interspecific cross. The locus on mouse chromosome 3, Mcl1, was homologous to MCL1 on human chromosome 1; the second locus, Mcl-rs, on mouse chromosome 5, may represent a pseudogene. The proximal long arm of human chromosome 1, where MCL1 is located, is duplicated and/or rearranged in a variety of preneoplastic and neoplastic diseases, including hematologic and solid tumors. Thus, MCL1 is a candidate gene for involvement in cancer. Animal model experiments lend further support to the function of MCL1. Rinkenberger et al. (2000) disrupted the Mcl1 locus in murine ES cells to determine the developmental roles of this Bcl2 family member. Deletion of Mcl1 resulted in periimplantation embryonic lethality. Homozygous Mcl1-deficient embryos did not implant in utero, but could be recovered at E3.5 to E4.0. Null blastocysts failed to hatch or attach in vitro, indicating a trophectoderm defect, although the inner cell mass could grow in culture. Of note, homozygous Mcl1-deficient blastocysts showed no evidence of increased apoptosis, but exhibited a delay in maturation beyond the precompaction stage. This model indicates that Mcl1 is essential for preimplantation development and implantation, and suggests that it has a function beyond regulating apoptosis.

It is appreciated that the abovementioned animal model for MCL1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Kozopas, K. M.; Yang, T.; Buchan, H. L.; Zhou, P.; Craig, R. W.: MCL1, a gene expressed in programmed myeloid cell differentiation, has sequence similarity to BCL2. Proc. Nat. Acad. Sci. 90: 3516-3520, 1993.; and
Bae, J.; Leo, C. P.; Hsu, S. Y.; Hsuch, A. J. W.: MCL-1S, a splicing variant of the antiapoptotic BCL-2 family member MCL-1, encodes a proapoptotic protein possessing only the BH3 domai.

Further studies establishing the function and utilities of MCL1 are found in John Hopkins OMIM database record ID 159552, and in sited publications numbered 780-783 listed in the bibliography section hereinbelow, which are also hereby incorporated by reference. Paired Basic Amino Acid Cleaving System 4 (PACE4, Accession NM_{—}138325) is another VGAM165 host target gene. PACE4 BINDING SITE is HOST TARGET binding site found in the 5′ untranslated region of mRNA encoded by PACE4, corresponding to a HOST TARGET binding site such as BINDING SITE I, BINDING SITE II or BINDING SITE III. Table 2 illustrates the complementarity of the nucleotide sequences of PACE4 BINDING SITE, designated SEQ ID:2434, to the nucleotide sequence of VGAM165 RNA, herein designated VGAM RNA, also designated SEQ ID:500.

Another function of VGAM165 is therefore inhibition of Paired Basic Amino Acid Cleaving System 4 (PACE4, Accession NM_{—}138325), a gene which processes hormone precursors by cleaving paired basic amino acids. Accordingly, utilities of VGAM165 include diagnosis, prevention and treatment of diseases and clinical conditions associated with PACE4. The function of PACE4 and its association with various diseases and clinical conditions, has been established by previous studies, as described hereinabove with reference to VGAM86. Podocalyxin-like (PODXL, Accession NM_{—}005397) is another VGAM165 host target gene. PODXL BINDING SITE is HOST TARGET binding site found in the 3′ untranslated region of mRNA encoded by PODXL, corresponding to a HOST TARGET binding site such as BINDING SITE I, BINDING SITE II or BINDING SITE III. Table 2 illustrates the complementarity of the nucleotide sequences of PODXL BINDING SITE, designated SEQ ID:1206, to the nucleotide sequence of VGAM165 RNA, herein designated VGAM RNA, also designated SEQ ID:500.

Another function of VGAM165 is therefore inhibition of Podocalyxin-like (PODXL, Accession NM_{—}005397), a gene which is an antiadhesin. Accordingly, utilities of VGAM165 include diagnosis, prevention and treatment of diseases and clinical conditions associated with PODXL. The function of PODXL has been established by previous studies. The renal glomerular epithelial cell, or podocyte, is a highly differentiated cell with characteristic interdigitating foot processes covering the outer aspect of the glomerular basement membrane. The foot processes are covered on their apical suface with a polyanionic glycocalyx, which is an essential element of the glomerular filter. Podocalyxin, a sialoglycoprotein, is thought to be a major component of this glycocalyx. By screening human renal cortex and heart cDNA libraries with a rabbit podocalyxin-like protein-1 (PCLP1) cDNA, Kershaw et al. (1997) cloned cDNAs encoding human PCLP, or PODXL. Northern blot analysis revealed that PODXL is expressed as a major 5.9-kb transcript and minor 4.4- and 9.6-kb transcripts in various tissues, with highest expression in kidney, pancreas, and heart. The predicted 528-amino acid protein has a 21-amino acid signal peptide, a transmembrane domain, and a highly acidic intracellular domain. The amino acid sequence of human PODXL is 48% identical to that of rabbit PCLP1, with 96% identity in the transmembrane and intracellular domains. The calculated molecular mass of PODXL is 54 kD. Western blot analysis of renal glomerular extracts showed that monoclonal antibodies against human PODXL recognize a 160/165-kD human PODXL doublet, rat podocalyxin, and rabbit PCLP1. Kershaw et al. (1997) suggested that the discrepancy between the calculated and observed masses of human PODXL is due to posttranslational modifications. By immunofluorescence of human kidney sections using antibodies against PODXL, Kershaw et al. (1997) found intense vascular endothelial cell and glomerular staining. Kershaw et al. (1997) mapped the human PODXL gene to 7q32-q33 by fluorescence in situ hybridization.

Full details of the abovementioned studies are described in the following publications, the disclosure of which are hereby incorporated by reference:
Kershaw, D. B.; Beck, S. G.; Wharram, B. L.; Wiggins, J. E.; Goyal, M.; Thomas, P. E.; Wiggins, R. C.: Molecular cloning and characterization of human podocalyxin-like protein: orthologous relationship to rabbit PCLP1 and rat podocalyxin. J. Biol. Chem. 272: 15708-15714, 1997.; and
Kershaw, D. B.; Wiggins, J. E.; Wharram, B. L.; Wiggins, R. C.: Assignment of the human podocalyxin-like protein (PODXL) gene to 7q32-q33. Genomics 45: 239-240, 1997.

Further studies establishing the function and utilities of PODXL are found in John Hopkins OMIM database record ID 602632, and in sited publications numbered 1971-1972 listed in the bibliography section hereinbelow, which are also hereby incorporated by reference. Tumor Necrosis Factor (ligand) Super family, Member 8 (TNFSF8, Accession NM_{—}001244) is another VGAM165 host target gene. TNFSF8 BINDING SITE is HOST TARGET binding site found in the 3′ untranslated region of mRNA encoded by TNFSF8, corresponding to a HOST TARGET binding site such as BINDING SITE I, BINDING SITE II or BINDING SITE III. Table 2 illustrates the complementarity of the nucleotide sequences of TNFSF8 BINDING SITE, designated SEQ ID:811, to the nucleotide sequence of VGAM165 RNA, herein designated VGAM RNA, also designated SEQ ID:500.

Another function of VGAM165 is therefore inhibition of Tumor Necrosis Factor (ligand) Super family, Member 8 (TNFSF8, Accession NM_{—}001244), a gene which cytokine that binds to tnfrsf8/cd30. induces proliferation of t cells. Accordingly, utilities of VGAM165 include diagnosis, prevention and treatment of diseases and clinical conditions associated with TNFSF8. The function of TNFSF8 has been established by previous studies. CD30 (TNFRSF8; 153243), a member of the tumor necrosis factor (TNF; OMIM Ref. No. TNF-alpha 191160) receptor super family, is a surface antigen used as a clinical marker for Hodgkin lymphoma and related hematologic malignancies. By performing an expression cloning screen using a chimeric protein containing the extracellular domain of CD30 as a probe, Smith et al. (1993) identified murine cells expressing a CD30 ligand. They isolated the corresponding mouse cDNA and used it to recover a homologous human cDNA from a peripheral blood T-cell (PBT) library. The predicted 234-amino acid human CD30L (CD30 ligand) protein is 72% identical to mouse Cd301. CD30L has the characteristics of a type II membrane protein, with no apparent signal peptide and a transmembrane domain followed by a C-terminal extracellular domain. The C-terminal receptor-binding region of CD30L shares sequence similarity with other members of the TNF family, including TNF-alpha, TNF-beta (OMIM Ref. No. 153440), and CD40LG (OMIM Ref. No. 300386). Although it has a predicted molecular weight of 26 kD, recombinant CD30L expressed in mammalian cells migrated at 40 kD by SDS-PAGE. Smith et al. (1993) attributed this discrepancy to extensive glycosylation of the extracellular domain in vivo. The recombinant human CD30L enhanced the proliferation of CD3 (OMIM Ref. No. 186790)-activated T cells, but induced differential responses, including cell death, in several CD30-positive lymphoma-derived cell lines. Northern blot analysis suggested that CD30L expression is limited to specifically induced T cells and monocytes/macrophages. Croager and Abraham (1997) determined that the CD30L gene contains 4 exons and spans more than 17.1 kb. Cerutti et al. (2000) noted that CD153 is expressed on the surface of B cells and found that this expression is up-regulated upon CD154 (OMIM Ref. No. CD40LG), IL4 (OMIM Ref. No. 147780), and B-cell receptor engagement. In these cells, engagement of CD153 by T cell CD30 inhibits immunoglobulin class switch recognition as well as IgG, IgA, and IgE production, suggesting that this ‘reverse signaling’ modulates the CD154-dependent switching of B cells into the pool producing IgG, IgA, and IgE. By analysis of an interspecific backcross, Smith et al. (1993) mapped the Cd301 gene to the proximal region of mouse chromosome 4. These authors used fluorescence in situ hybridization to map the human CD30L gene to 9q33.

Full details of the abovementioned studies are described in the following publications, the disclosure of which are hereby incorporated by reference:
Cerutti, A.; Schaffer, A.; Goodwin, R. G.; Shah, S.; Zan, H.; Ely, S.; Casali, P.: Engagement of CD153 (CD30 ligand) by CD30-positive T cells inhibits class switch DNA recombination and antibody production in human IgD-positive IgM-positive B cells. J. Immun. 165: 786-794, 2000.; and
Croager, E. J.; Abraham, L. J.: Characterisation of the human CD30 ligand gene structure. Biochim. Biophys. Acta 1353: 231-235, 1997.

Further studies establishing the function and utilities of TNFSF8 are found in John Hopkins OMIM database record ID 603875, and in sited publications numbered 1733-173 and 1736 listed in the bibliography section hereinbelow, which are also hereby incorporated by reference. ATPase, (Na+)/K+ Transporting, Beta 4 Polypeptide (ATP1B4, Accession NM_{—}012069) is another VGAM165 host target gene. ATP1B4 BINDING SITE is HOST TARGET binding site found in the 3′ untranslated region of mRNA encoded by ATP1B4, corresponding to a HOST TARGET binding site such as BINDING SITE I, BINDING SITE II or BINDING SITE III. Table 2 illustrates the complementarity of the nucleotide sequences of ATP1B4 BINDING SITE, designated SEQ ID:1401, to the nucleotide sequence of VGAM165 RNA, herein designated VGAM RNA, also designated SEQ ID:500.

Another function of VGAM165 is therefore inhibition of ATPase, (Na+)/K+ Transporting, Beta 4 Polypeptide (ATP1B4, Accession NM_{—}012069). Accordingly, utilities of VGAM165 include diagnosis, prevention and treatment of diseases and clinical conditions associated with ATP1B4. CDCl4 Cell Division Cycle 14 Homolog A (S. cerevisiae) (CDCl4A, Accession NM_{—}003672) is another VGAM165 host target gene. CDCl4A BINDING SITE is HOST TARGET binding site found in the 3′ untranslated region of mRNA encoded by CDCl4A, corresponding to a HOST TARGET binding site such as BINDING SITE I, BINDING SITE II or BINDING SITE III. Table 2 illustrates the complementarity of the nucleotide sequences of CDCl4A BINDING SITE, designated SEQ ID: 1047, to the nucleotide sequence of VGAM165 RNA, herein designated VGAM RNA, also designated SEQ ID:500.

Another function of VGAM165 is therefore inhibition of CDCl4 Cell Division Cycle 14 Homolog A (S. cerevisiae) (CDCl4A, Accession NM_{—}003672). Accordingly, utilities of VGAM165 include diagnosis, prevention and treatment of diseases and clinical conditions associated with CDCl4A. DKFZP434C171 (Accession NM_{—}015621) is another VGAM165 host target gene. DKFZP434C171 BINDING SITE is HOST TARGET binding site found in the 3′ untranslated region of mRNA encoded by DKFZP434C171, corresponding to a HOST TARGET binding site such as BINDING SITE I, BINDING SITE II or BINDING SITE III. Table 2 illustrates the complementarity of the nucleotide sequences of DKFZP434C171 BINDING SITE, designated SEQ ID:1641, to the nucleotide sequence of VGAM165 RNA, herein designated VGAM RNA, also designated SEQ ID:500.

Another function of VGAM165 is therefore inhibition of DKFZP434C171 (Accession NM_{—}015621). Accordingly, utilities of VGAM165 include diagnosis, prevention and treatment of diseases and clinical conditions associated with DKFZP434C171. Egf-like Module Containing, Mucin-like, Hormone Receptor-like Sequence 2 (EMR2, Accession NM_{—}013447) is another VGAM165 host target gene. EMR2 BINDING SITE is HOST TARGET binding site found in the 3′ untranslated region of mRNA encoded by EMR2, corresponding to a HOST TARGET binding site such as BINDING SITE I, BINDING SITE II or BINDING SITE III. Table 2 illustrates the complementarity of the nucleotide sequences of EMR2 BINDING SITE, designated SEQ ID: 1453, to the nucleotide sequence of VGAM165 RNA, herein designated VGAM RNA, also designated SEQ ID:500.

Another function of VGAM165 is therefore inhibition of Egf-like Module Containing, Mucin-like, Hormone Receptor-like Sequence 2 (EMR2, Accession NM_{—}013447). Accordingly, utilities of VGAM165 include diagnosis, prevention and treatment of diseases and clinical conditions associated with EMR2. FYVE and Coiled-coil Domain Containing 1 (FYCO1, Accession NM_{—}024513) is another VGAM165 host target gene. FYCO1 BINDING SITE is HOST TARGET binding site found in the 3′ untranslated region of mRNA encoded by FYCO1, corresponding to a HOST TARGET binding site such as BINDING SITE I, BINDING SITE II or BINDING SITE III. Table 2 illustrates the complementarity of the nucleotide sequences of FYCO1 BINDING SITE, designated SEQ ID:2063, to the nucleotide sequence of VGAM165 RNA, herein designated VGAM RNA, also designated SEQ ID:500.

Another function of VGAM165 is therefore inhibition of FYVE and Coiled-coil Domain Containing 1 (FYCO1, Accession NM_{—}024513). Accordingly, utilities of VGAM165 include diagnosis, prevention and treatment of diseases and clinical conditions associated with FYCO1. Golgi Associated, Gamma Adaptin Ear Containing, ARF Binding Protein 2 (GGA2, Accession NM_{—}138640) is another VGAM165 host target gene. GGA2 BINDING SITE1 and GGA2 BINDING SITE2 are HOST TARGET binding sites found in untranslated regions of mRNA encoded by GGA2, corresponding to HOST TARGET binding sites such as BINDING SITE I, BINDING SITE II or BINDING SITE III. Table 2 illustrates the complementarity of the nucleotide sequences of GGA2 BINDING SITE1 and GGA2 BINDING SITE2, designated SEQ ID:2448 and SEQ ID:1604 respectively, to the nucleotide sequence of VGAM165 RNA, herein designated VGAM RNA, also designated SEQ ID:500.

Another function of VGAM165 is therefore inhibition of Golgi Associated, Gamma Adaptin Ear Containing, ARF Binding Protein 2 (GGA2, Accession NM_{—}138640). Accordingly, utilities of VGAM165 include diagnosis, prevention and treatment of diseases and clinical conditions associated with GGA2. ZFP100 (Accession XM_{—}046390) is another VGAM165 host target gene. ZFP100 BINDING SITE is HOST TARGET binding site found in the 3′ untranslated region of mRNA encoded by ZFP100, corresponding to a HOST TARGET binding site such as BINDING SITE I, BINDING SITE II or BINDING SITE III. Table 2 illustrates the complementarity of the nucleotide sequences of ZFP100 BINDING SITE, designated SEQ ID:2877, to the nucleotide sequence of VGAM165 RNA, herein designated VGAM RNA, also designated SEQ ID:500.

Another function of VGAM165 is therefore inhibition of ZFP100 (Accession XM_{—}046390). Accordingly, utilities of VGAM165 include diagnosis, prevention and treatment of diseases and clinical conditions associated with ZFP100. LOC64744 (Accession XM_{—}029830) is another VGAM165 host target gene. LOC64744 BINDING SITE is HOST TARGET binding site found in the 3′ untranslated region of mRNA encoded by LOC64744, corresponding to a HOST TARGET binding site such as BINDING SITE I, BINDING SITE II or BINDING SITE III. Table 2 illustrates the complementarity of the nucleotide sequences of LOC64744 BINDING SITE, designated SEQ ID:2607, to the nucleotide sequence of VGAM165 RNA, herein designated VGAM RNA, also designated SEQ ID:500.

Another function of VGAM165 is therefore inhibition of LOC64744 (Accession XM_{—}029830). Accordingly, utilities of VGAM165 include diagnosis, prevention and treatment of diseases and clinical conditions associated with LOC64744. FIG. 1 further provides a conceptual description of a novel bioinformatically detected viral gene of the present invention, referred to here as Viral Genomic Address Messenger 166 (VGAM166) viral gene, which modulates expression of respective host target genes thereof, the function and utility of which host target genes is known in the art.

VGAM166 is a novel bioinformatically detected regulatory, non protein coding, viral micro RNA (miRNA) gene. The method by which VGAM166 was detected is described hereinabove with reference to FIGS. 1-8.

VGAM166 gene, herein designated VGAM GENE, is a viral gene contained in the genome of Vaccinia Virus. VGAM166 host target gene, herein designated VGAM HOST TARGET GENE, is a human gene contained in the human genome.

VGAM166 gene encodes a VGAM166 precursor RNA, herein designated VGAM PRECURSOR RNA. Similar to other miRNA genes, and unlike most ordinary genes, VGAM166 precursor RNA does not encode a protein. A nucleotide sequence identical or highly similar to the nucleotide sequence of VGAM166 precursor RNA is designated SEQ ID:152, and is provided hereinbelow with reference to the sequence listing part. Nucleotide sequence SEQ ID:152 is located at position 83280 relative to the genome of Vaccinia Virus.

VGAM166 precursor RNA folds onto itself, forming VGAM166 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM166 folded precursor RNA into VGAM166 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM166 RNA is designated SEQ ID:501, and is provided hereinbelow with reference to the sequence listing part.

VGAM166 host target gene, herein designated VGAM HOST TARGET GENE, encodes a corresponding messenger RNA, VGAM166 host target RNA, herein designated VGAM HOST TARGET RNA. VGAM166 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM166 RNA, herein designated VGAM RNA, binds complementarily to one or more host target binding sites located in untranslated regions of VGAM166 host target RNA, herein designated VGAM HOST TARGET RNA. This complementary binding is due to the fact that the nucleotide sequence of VGAM166 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM166 RNA, herein designated VGAM RNA, may have a different number of host target binding sites in untranslated regions of a VGAM166 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM166 RNA, herein designated VGAM RNA, to host target binding sites on VGAM166 host target RNA, herein designated VGAM HOST TARGET RNA, such as BINDING SITE I, BINDING SITE II and BINDING SITE III, inhibits translation of VGAM166 host target RNA into VGAM166 host target protein, herein designated VGAM HOST TARGET PROTEIN. VGAM host target protein is therefore outlined by a broken line.

It is appreciated that VGAM166 host target gene, herein designated VGAM HOST TARGET GENE, in fact represents a plurality of VGAM166 host target genes. The mRNA of each one of this plurality of VGAM166 host target genes comprises one or more host target binding sites, each having a nucleotide sequence which is at least partly complementary to VGAM166 RNA, herein designated VGAM RNA, and which when bound by VGAM166 RNA causes inhibition of translation of respective one or more VGAM166 host target proteins.

It is further appreciated by one skilled in the art that the mode of translational inhibition illustrated by FIG. 1 with specific reference to translational inhibition exerted by VGAM166 gene, herein designated VGAM GENE, on one or more VGAM166 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM166 is inhibition of expression of host target genes, as part of a novel viral mechanism of attacking a host. Accordingly, utilities of VGAM166 include diagnosis, prevention and treatment of viral infection by Vaccinia Virus. Specific functions, and accordingly utilities, of VGAM166 correlate with, and may be deduced from, the identity of the host target genes which VGAM166 binds and inhibits, and the function of these host target genes, as elaborated hereinbelow.

Nucleotide sequences of the VGAM166 precursor RNA, herein designated VGAM PRECURSOR RNA, and of the ‘diced’ VGAM166 RNA, herein designated VGAM RNA, and a schematic representation of the secondary folding of VGAM166 folded precursor RNA, herein designated VGAM FOLDED PRECURSOR RNA, of VGAM166 are further described hereinbelow with reference to Table 1.

Nucleotide sequences of host target binding sites, such as BINDING SITE-I, BINDING SITE-II and BINDING SITE-III of FIG. 1, found on VGAM166 host target RNA, and schematic representation of the complementarity of each of these host target binding sites to VGAM166 RNA, herein designated VGAM RNA, are described hereinbelow with reference to Table 2.

As mentioned hereinabove with reference to FIG. 1, a function of VGAM166 gene, herein designated VGAM is inhibition of expression of VGAM166 target genes. It is appreciated that specific functions, and accordingly utilities, of VGAM166 correlate with, and may be deduced from, the identity of the target genes which VGAM166 binds and inhibits, and the function of these target genes, as elaborated hereinbelow.

MEF-2 (Accession XM_{—}034883) is a VGAM166 host target gene. MEF-2 BINDING SITE is HOST TARGET binding site found in the 3′ untranslated region of mRNA encoded by MEF-2, corresponding to a HOST TARGET binding site such as BINDING SITE I, BINDING SITE II or BINDING SITE III. Table 2 illustrates the complementarity of the nucleotide sequences of MEF-2 BINDING SITE, designated SEQ ID:2688, to the nucleotide sequence of VGAM166 RNA, herein designated VGAM RNA, also designated SEQ ID:501.

A function of VGAM166 is therefore inhibition of MEF-2 (Accession XM_{—}034883). Accordingly, utilities of VGAM166 include diagnosis, prevention and treatment of diseases and clinical conditions associated with MEF-2. LOC146184 (Accession XM_{—}096942) is another VGAM166 host target gene. LOC146184 BINDING SITE is HOST TARGET binding site found in the 5′ untranslated region of mRNA encoded by LOC146184, corresponding to a HOST TARGET binding site such as BINDING SITE I, BINDING SITE II or BINDING SITE III. Table 2 illustrates the complementarity of the nucleotide sequences of LOC146184 BINDING SITE, designated SEQ ID:3281, to the nucleotide sequence of VGAM166 RNA, herein designated VGAM RNA, also designated SEQ ID:501.

Another function of VGAM166 is therefore inhibition of LOC146184 (Accession XM_{—}096942). Accordingly, utilities of VGAM166 include diagnosis, prevention and treatment of diseases and clinical conditions associated with LOC146184. FIG. 1 further provides a conceptual description of a novel bioinformatically detected viral gene of the present invention, referred to here as Viral Genomic Address Messenger 167 (VGAM167) viral gene, which modulates expression of respective host target genes thereof, the function and utility of which host target genes is known in the art.

VGAM167 is a novel bioinformatically detected regulatory, non protein coding, viral micro RNA (miRNA) gene. The method by which VGAM167 was detected is described hereinabove with reference to FIGS. 1-8.

VGAM167 gene, herein designated VGAM GENE, is a viral gene contained in the genome of Vaccinia Virus. VGAM167 host target gene, herein designated VGAM HOST TARGET GENE, is a human gene contained in the human genome.

VGAM167 gene encodes a VGAM167 precursor RNA, herein designated VGAM PRECURSOR RNA. Similar to other miRNA genes, and unlike most ordinary genes, VGAM167 precursor RNA does not encode a protein. A nucleotide sequence identical or highly similar to the nucleotide sequence of VGAM167 precursor RNA is designated SEQ ID:153, and is provided hereinbelow with reference to the sequence listing part. Nucleotide sequence SEQ ID:153 is located at position 85141 relative to the genome of Vaccinia Virus.

VGAM167 precursor RNA folds onto itself, forming VGAM167 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM167 folded precursor RNA into VGAM167 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM167 RNA is designated SEQ ID:502, and is provided hereinbelow with reference to the sequence listing part.

VGAM167 host target gene, herein designated VGAM HOST TARGET GENE, encodes a corresponding messenger RNA, VGAM167 host target RNA, herein designated VGAM HOST TARGET RNA. VGAM167 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM167 RNA, herein designated VGAM RNA, binds complementarily to one or more host target binding sites located in untranslated regions of VGAM167 host target RNA, herein designated VGAM HOST TARGET RNA. This complementary binding is due to the fact that the nucleotide sequence of VGAM167 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM167 RNA, herein designated VGAM RNA, may have a different number of host target binding sites in untranslated regions of a VGAM167 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM167 RNA, herein designated VGAM RNA, to host target binding sites on VGAM167 host target RNA, herein designated VGAM HOST TARGET RNA, such as BINDING SITE I, BINDING SITE II and BINDING SITE III, inhibits translation of VGAM167 host target RNA into VGAM167 host target protein, herein designated VGAM HOST TARGET PROTEIN. VGAM host target protein is therefore outlined by a broken line.

It is appreciated that VGAM167 host target gene, herein designated VGAM HOST TARGET GENE, in fact represents a plurality of VGAM167 host target genes. The mRNA of each one of this plurality of VGAM167 host target genes comprises one or more host target binding sites, each having a nucleotide sequence which is at least partly complementary to VGAM167 RNA, herein designated VGAM RNA, and which when bound by VGAM167 RNA causes inhibition of translation of respective one or more VGAM167 host target proteins.

It is further appreciated by one skilled in the art that the mode of translational inhibition illustrated by FIG. 1 with specific reference to translational inhibition exerted by VGAM167 gene, herein designated VGAM GENE, on one or more VGAM167 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM167 is inhibition of expression of host target genes, as part of a novel viral mechanism of attacking a host. Accordingly, utilities of VGAM167 include diagnosis, prevention and treatment of viral infection by Vaccinia Virus. Specific functions, and accordingly utilities, of VGAM167 correlate with, and may be deduced from, the identity of the host target genes which VGAM167 binds and inhibits, and the function of these host target genes, as elaborated hereinbelow.

Nucleotide sequences of the VGAM167 precursor RNA, herein designated VGAM PRECURSOR RNA, and of the ‘diced’ VGAM167 RNA, herein designated VGAM RNA, and a schematic representation of the secondary folding of VGAM167 folded precursor RNA, herein designated VGAM FOLDED PRECURSOR RNA, of VGAM167 are further described hereinbelow with reference to Table 1.

Nucleotide sequences of host target binding sites, such as BINDING SITE-I, BINDING SITE-II and BINDING SITE-III of FIG. 1, found on VGAM167 host target RNA, and schematic representation of the complementarity of each of these host target binding sites to VGAM167 RNA, herein designated VGAM RNA, are described hereinbelow with reference to Table 2.

As mentioned hereinabove with reference to FIG. 1, a function of VGAM167 gene, herein designated VGAM is inhibition of expression of VGAM167 target genes. It is appreciated that specific functions, and accordingly utilities, of VGAM167 correlate with, and may be deduced from, the identity of the target genes which VGAM167 binds and inhibits, and the function of these target genes, as elaborated hereinbelow.

Cadherin 5, Type 2, VE-cadherin (vascular epithelium) (CDH5, Accession NM_{—}001795) is a VGAM167 host target gene. CDH5 BINDING SITE is HOST TARGET binding site found in the 3′ untranslated region of mRNA encoded by CDH5, corresponding to a HOST TARGET binding site such as BINDING SITE I, BINDING SITE II or BINDING SITE III. Table 2 illustrates the complementarity of the nucleotide sequences of CDH5 BINDING SITE, designated SEQ ID:858, to the nucleotide sequence of VGAM167 RNA, herein designated VGAM RNA, also designated SEQ ID:502.

A function of VGAM167 is therefore inhibition of Cadherin 5, Type 2, VE-cadherin (vascular epithelium) (CDH5, Accession NM_{—}001795), a gene which associates with alpha-catenin forming a link to the cytoskeleton. Accordingly, utilities of VGAM167 include diagnosis, prevention and treatment of diseases and clinical conditions associated with CDH5. The function of CDH5 and its association with various diseases and clinical conditions, has been established by previous studies, as described hereinabove with reference to VGAM111. LNK (Accession NM_{—}005475) is another VGAM167 host target gene. LNK BINDING SITE is HOST TARGET binding site found in the 3′ untranslated region of mRNA encoded by LNK, corresponding to a HOST TARGET binding site such as BINDING SITE I, BINDING SITE II or BINDING SITE III. Table 2 illustrates the complementarity of the nucleotide sequences of LNK BINDING SITE, designated SEQ ID:1216, to the nucleotide sequence of VGAM167 RNA, herein designated VGAM RNA, also designated SEQ ID:502.

Another function of VGAM167 is therefore inhibition of LNK (Accession NM_{—}005475), a gene which links T-cell receptor activation signal to phospholipase c-gamma-1, grb-2 and phosphatidylinositol 3-kinase (by similarity). Accordingly, utilities of VGAM167 include diagnosis, prevention and treatment of diseases and clinical conditions associated with LNK. The function of LNK has been established by previous studies. By PCR using primers based on the rat Lnk sequence and by screening a Jurkat cDNA library, L1 et al. (2000) obtained a cDNA encoding human LNK. Sequence analysis predicted that the 575-amino acid LNK protein contains an N-terminal proline-rich region, a pleckstrin homology (PH) domain, and an Src homology 2 (SH2) domain; the PH and SH2 domains are similar to those of the APS protein. Northern blot analysis detected low expression of a 6.8-kb LNK transcript in various lymphoid cell lines. Confocal fluorescence microscopy showed that the majority of LNK is located in the juxtanuclear region with some found near the plasma membrane. Immunoprecipitation analysis demonstrated that LNK is phosphorylated by LCK (OMIM Ref. No. 153390) but not by SYK (OMIM Ref. No. 600085) and that LNK binds to the tyrosine-phosphorylated TCR zeta chain via its SH2 domain. Functional analysis indicated that LNK inhibits the activation of NFAT (see OMIM Ref. No. 600489) in stimulated T cells Animal model experiments lend further support to the function of LNK. Takaki et al. (2000) generated Lnk-deficient mice and found that although they had unimpaired T-cell development in thymus, pre-B and immature B cells accumulated in enlarged spleens. In bone marrow, there was also an increase in B-lineage cells, reflecting enhanced production of B-cell progenitors due in part to hypersensitivity to SCF (KITLG; 184745) in the presence or absence of IL7 (OMIM Ref. No. 146660). Western blot analysis showed that mouse Lnk is actually a 68-kD protein

It is appreciated that the abovementioned animal model for LNK is acknowledged by those skilled in the art as a scientifically valid animal model, as can be further appreciated from the publications sited hereinbelow.

Full details of the abovementioned studies are described in the following publications, the disclosure of which are hereby incorporated by reference:
Li, Y.; He, X.; Schembri-King, J.; Jakes, S.; Hayashi, J.: Cloning and characterization of human Lnk, an adaptor protein with pleckstrin homology and Src homology 2 domains that can inhibit T cell activation. J. Immun. 164: 5199-5206, 2000.; and
Takaki, S.; Sauer, K.; Iritani, B. M.; Chien, S.; Ebihara, Y.; Tsuji, K.; Takatsu, K.; Perlmutter, R. M.: Control of B cell production by the adaptor protein Lnk: definition of a conserve.

Further studies establishing the function and utilities of LNK are found in John Hopkins OMIM database record ID 605093, and in sited publications numbered 1582-1583 listed in the bibliography section hereinbelow, which are also hereby incorporated by reference. Natriuretic Peptide Receptor B/guanylate Cyclase B (atrionatriuretic peptide receptor B) (NPR2, Accession NM_{—}000907) is another VGAM167 host target gene. NPR2 BINDING SITE is HOST TARGET binding site found in the 3′ untranslated region of mRNA encoded by NPR2, corresponding to a HOST TARGET binding site such as BINDING SITE I, BINDING SITE II or BINDING SITE III. Table 2 illustrates the complementarity of the nucleotide sequences of NPR2 BINDING SITE, designated SEQ ID:786, to the nucleotide sequence of VGAM167 RNA, herein designated VGAM RNA, also designated SEQ ID:502.

Another function of VGAM167 is therefore inhibition of Natriuretic Peptide Receptor B/guanylate Cyclase B (atrionatriuretic peptide receptor B) (NPR2, Accession NM_{—}000907). Accordingly, utilities of VGAM167 include diagnosis, prevention and treatment of diseases and clinical conditions associated with NPR2. CLLD8 (Accession NM_{—}031915) is another VGAM167 host target gene. CLLD8 BINDING SITE is HOST TARGET binding site found in the 3′ untranslated region of mRNA encoded by CLLD8, corresponding to a HOST TARGET binding site such as BINDING SITE I, BINDING SITE II or BINDING SITE III. Table 2 illustrates the complementarity of the nucleotide sequences of CLLD8 BINDING SITE, designated SEQ ID:2219, to the nucleotide sequence of VGAM167 RNA, herein designated VGAM RNA, also designated SEQ ID:502.

Another function of VGAM167 is therefore inhibition of CLLD8 (Accession NM_{—}031915). Accordingly, utilities of VGAM167 include diagnosis, prevention and treatment of diseases and clinical conditions associated with CLLD8. FLJ22301 (Accession NM_{—}024836) is another VGAM167 host target gene. FLJ22301 BINDING SITE is HOST TARGET binding site found in the 3′ untranslated region of mRNA encoded by FLJ22301, corresponding to a HOST TARGET binding site such as BINDING SITE I, BINDING SITE II or BINDING SITE III. Table 2 illustrates the complementarity of the nucleotide sequences of FLJ22301 BINDING SITE, designated SEQ ID:2094, to the nucleotide sequence of VGAM167 RNA, herein designated VGAM RNA, also designated SEQ ID:502.

Another function of VGAM167 is therefore inhibition of FLJ22301 (Accession NM_{—}024836). Accordingly, utilities of VGAM167 include diagnosis, prevention and treatment of diseases and clinical conditions associated with FLJ22301. FLJ22833 (Accession NM_{—}022837) is another VGAM167 host target gene. FLJ22833 BINDING SITE is HOST TARGET binding site found in the 3′ untranslated region of mRNA encoded by FLJ22833, corresponding to a HOST TARGET binding site such as BINDING SITE I, BINDING SITE II or BINDING SITE III. Table 2 illustrates the complementarity of the nucleotide sequences of FLJ22833 BINDING SITE, designated SEQ ID:2016, to the nucleotide sequence of VGAM167 RNA, herein designated VGAM RNA, also designated SEQ ID:502.

Another function of VGAM167 is therefore inhibition of FLJ22833 (Accession NM_{—}022837). Accordingly, utilities of VGAM167 include diagnosis, prevention and treatment of diseases and clinical conditions associated with FLJ22833. KIAA0125 (Accession NM_{—}014792) is another VGAM167 host target gene. KIAA0125 BINDING SITE1 through KIAA0125 BINDING SITES are HOST TARGET binding sites found in untranslated regions of mRNA encoded by KIAA0125, corresponding to HOST TARGET binding sites such as BINDING SITE I, BINDING SITE II or BINDING SITE III. Table 2 illustrates the complementarity of the nucleotide sequences of KIAA0125 BINDING SITE1 through KIAA0125 BINDING SITES, designated SEQ ID:1548, SEQ ID:1549, SEQ ID:1550, SEQ ID:1551 and SEQ ID:1552 respectively, to the nucleotide sequence of VGAM167 RNA, herein designated VGAM RNA, also designated SEQ ID:502.

Another function of VGAM167 is therefore inhibition of KIAA0125 (Accession NM_{—}014792). Accordingly, utilities of VGAM167 include diagnosis, prevention and treatment of diseases and clinical conditions associated with KIAA0125. LanC Lantibiotic Synthetase Component C-like 2 (bacterial) (LANCL2, Accession NM_{—}018697) is another VGAM167 host target gene. LANCL2 BINDING SITE is HOST TARGET binding site found in the 3′ untranslated region of mRNA encoded by LANCL2, corresponding to a HOST TARGET binding site such as BINDING SITE I, BINDING SITE II or BINDING SITE III. Table 2 illustrates the complementarity of the nucleotide sequences of LANCL2 BINDING SITE, designated SEQ ID:1860, to the nucleotide sequence of VGAM167 RNA, herein designated VGAM RNA, also designated SEQ ID:502.

Another function of VGAM167 is therefore inhibition of LanC Lantibiotic Synthetase Component C-like 2 (bacterial) (LANCL2, Accession NM_{—}018697). Accordingly, utilities of VGAM167 include diagnosis, prevention and treatment of diseases and clinical conditions associated with LANCL2. Seizure Related 6 Homolog (mouse) (SEZ6, Accession XM_{—}058869) is another VGAM167 host target gene. SEZ6 BINDING SITE is HOST TARGET binding site found in the 3′ untranslated region of mRNA encoded by SEZ6, corresponding to a HOST TARGET binding site such as BINDING SITE I, BINDING SITE II or BINDING SITE III. Table 2 illustrates the complementarity of the nucleotide sequences of SEZ6 BINDING SITE, designated SEQ ID:2997, to the nucleotide sequence of VGAM167 RNA, herein designated VGAM RNA, also designated SEQ ID:502.

Another function of VGAM167 is therefore inhibition of Seizure Related 6 Homolog (mouse) (SEZ6, Accession XM_{—}058869). Accordingly, utilities of VGAM167 include diagnosis, prevention and treatment of diseases and clinical conditions associated with SEZ6. Sorting Nexin 10 (SNX10, Accession NM_{—}013322) is another VGAM167 host target gene. SNX10 BINDING SITE is HOST TARGET binding site found in the 3′ untranslated region of mRNA encoded by SNX10, corresponding to a HOST TARGET binding site such as BINDING SITE I, BINDING SITE II or BINDING SITE III. Table 2 illustrates the complementarity of the nucleotide sequences of SNX10 BINDING SITE, designated SEQ ID: 1442, to the nucleotide sequence of VGAM167 RNA, herein designated VGAM RNA, also designated SEQ ID:502.

Another function of VGAM167 is therefore inhibition of Sorting Nexin 10 (SNX10, Accession NM_{—}013322). Accordingly, utilities of VGAM167 include diagnosis, prevention and treatment of diseases and clinical conditions associated with SNX10. Tumor Necrosis Factor Receptor Super family, Member 21 (TNFRSF21, Accession NM_{—}014452) is another VGAM167 host target gene. TNFRSF21 BINDING SITE is HOST TARGET binding site found in the 3′ untranslated region of mRNA encoded by TNFRSF21, corresponding to a HOST TARGET binding site such as BINDING SITE I, BINDING SITE II or BINDING SITE III. Table 2 illustrates the complementarity of the nucleotide sequences of TNFRSF21 BINDING SITE, designated SEQ ID:1502, to the nucleotide sequence of VGAM167 RNA, herein designated VGAM RNA, also designated SEQ ID:502.

Another function of VGAM167 is therefore inhibition of Tumor Necrosis Factor Receptor Super family, Member 21 (TNFRSF21, Accession NM_{—}014452). Accordingly, utilities of VGAM167 include diagnosis, prevention and treatment of diseases and clinical conditions associated with TNFRSF21. Testis-specific Transcript, Y-linked 11 (TTTY11, Accession NM_{—}031929) is another VGAM167 host target gene. TTTY11 BINDING SITE is HOST TARGET binding site found in the 5′ untranslated region of mRNA encoded by TTTY11, corresponding to a HOST TARGET binding site such as BINDING SITE I, BINDING SITE II or BINDING SITE III. Table 2 illustrates the complementarity of the nucleotide sequences of TTTY11 BINDING SITE, designated SEQ ID:2221, to the nucleotide sequence of VGAM167 RNA, herein designated VGAM RNA, also designated SEQ ID:502.

Another function of VGAM167 is therefore inhibition of Testis-specific Transcript, Y-linked 11 (TTTY11, Accession NM_{—}031929). Accordingly, utilities of VGAM167 include diagnosis, prevention and treatment of diseases and clinical conditions associated with TTTY11. LOC143879 (Accession XM_{—}084666) is another VGAM167 host target gene. LOC143879 BINDING SITE is HOST TARGET binding site found in the 3′ untranslated region of mRNA encoded by LOC143879, corresponding to a HOST TARGET binding site such as BINDING SITE I, BINDING SITE II or BINDING SITE III. Table 2 illustrates the complementarity of the nucleotide sequences of LOC143879 BINDING SITE, designated SEQ ID:3063, to the nucleotide sequence of VGAM167 RNA, herein designated VGAM RNA, also designated SEQ ID:502.

Another function of VGAM167 is therefore inhibition of LOC143879 (Accession XM_{—}084666). Accordingly, utilities of VGAM167 include diagnosis, prevention and treatment of diseases and clinical conditions associated with LOC143879. LOC145439 (Accession XM_{—}085144) is another VGAM167 host target gene. LOC145439 BINDING SITE is HOST TARGET binding site found in the 5′ untranslated region of mRNA encoded by LOC145439, corresponding to a HOST TARGET binding site such as BINDING SITE I, BINDING SITE II or BINDING SITE III. Table 2 illustrates the complementarity of the nucleotide sequences of LOC145439 BINDING SITE, designated SEQ ID:3078, to the nucleotide sequence of VGAM167 RNA, herein designated VGAM RNA, also designated SEQ ID:502.

Another function of VGAM167 is therefore inhibition of LOC145439 (Accession XM_{—}085144). Accordingly, utilities of VGAM167 include diagnosis, prevention and treatment of diseases and clinical conditions associated with LOC145439. LOC147353 (Accession XM_{—}097227) is another VGAM167 host target gene. LOC147353 BINDING SITE is HOST TARGET binding site found in the 3′ untranslated region of mRNA encoded by LOC147353, corresponding to a HOST TARGET binding site such as BINDING SITE I, BINDING SITE II or BINDING SITE III. Table 2 illustrates the complementarity of the nucleotide sequences of LOC147353 BINDING SITE, designated SEQ ID:3293, to the nucleotide sequence of VGAM167 RNA, herein designated VGAM RNA, also designated SEQ ID:502.

Another function of VGAM167 is therefore inhibition of LOC147353 (Accession XM_{—}097227). Accordingly, utilities of VGAM167 include diagnosis, prevention and treatment of diseases and clinical conditions associated with LOC147353. LOC221584 (Accession XM_{—}168132) is another VGAM167 host target gene. LOC221584 BINDING SITE is HOST TARGET binding site found in the 5′ untranslated region of mRNA encoded by LOC221584, corresponding to a HOST TARGET binding site such as BINDING SITE I, BINDING SITE II or BINDING SITE III. Table 2 illustrates the complementarity of the nucleotide sequences of LOC221584 BINDING SITE, designated SEQ ID:3620, to the nucleotide sequence of VGAM167 RNA, herein designated VGAM RNA, also designated SEQ ID:502.

Another function of VGAM167 is therefore inhibition of LOC221584 (Accession XM_{—}168132). Accordingly, utilities of VGAM167 include diagnosis, prevention and treatment of diseases and clinical conditions associated with LOC221584. LOC257054 (Accession XM_{—}171010) is another VGAM167 host target gene. LOC257054 BINDING SITE is HOST TARGET binding site found in the 3′ untranslated region of mRNA encoded by LOC257054, corresponding to a HOST TARGET binding site such as BINDING SITE I, BINDING SITE II or BINDING SITE III. Table 2 illustrates the complementarity of the nucleotide sequences of LOC257054 BINDING SITE, designated SEQ ID:3682, to the nucleotide sequence of VGAM167 RNA, herein designated VGAM RNA, also designated SEQ ID:502.

Another function of VGAM167 is therefore inhibition of LOC257054 (Accession XM_{—}171010). Accordingly, utilities of VGAM167 include diagnosis, prevention and treatment of diseases and clinical conditions associated with LOC257054. LOC91145 (Accession XM_{—}036454) is another VGAM167 host target gene. LOC91145 BINDING SITE is HOST TARGET binding site found in the 5′ untranslated region of mRNA encoded by LOC91145, corresponding to a HOST TARGET binding site such as BINDING SITE I, BINDING SITE II or BINDING SITE III. Table 2 illustrates the complementarity of the nucleotide sequences of LOC91145 BINDING SITE, designated SEQ ID:2713, to the nucleotide sequence of VGAM167 RNA, herein designated VGAM RNA, also designated SEQ ID:502.

Another function of VGAM167 is therefore inhibition of LOC91145 (Accession XM_{—}036454). Accordingly, utilities of VGAM167 include diagnosis, prevention and treatment of diseases and clinical conditions associated with LOC91145. LOC93297 (Accession XM_{—}050370) is another VGAM167 host target gene. LOC93297 BINDING SITE is HOST TARGET binding site found in the 3′ untranslated region of mRNA encoded by LOC93297, corresponding to a HOST TARGET binding site such as BINDING SITE I, BINDING SITE II or BINDING SITE III. Table 2 illustrates the complementarity of the nucleotide sequences of LOC93297 BINDING SITE, designated SEQ ID:2929, to the nucleotide sequence of VGAM167 RNA, herein designated VGAM RNA, also designated SEQ ID:502.

Another function of VGAM167 is therefore inhibition of LOC93297 (Accession XM_{—}050370). Accordingly, utilities of VGAM167 include diagnosis, prevention and treatment of diseases and clinical conditions associated with LOC93297. FIG. 1 further provides a conceptual description of a novel bioinformatically detected viral gene of the present invention, referred to here as Viral Genomic Address Messenger 168 (VGAM168) viral gene, which modulates expression of respective host target genes thereof, the function and utility of which host target genes is known in the art.

VGAM168 is a novel bioinformatically detected regulatory, non protein coding, viral micro RNA (miRNA) gene. The method by which VGAM168 was detected is described hereinabove with reference to FIGS. 1-8.

VGAM168 gene, herein designated VGAM GENE, is a viral gene contained in the genome of Vaccinia Virus. VGAM168 host target gene, herein designated VGAM HOST TARGET GENE, is a human gene contained in the human genome.

VGAM168 gene encodes a VGAM168 precursor RNA, herein designated VGAM PRECURSOR RNA. Similar to other miRNA genes, and unlike most ordinary genes, VGAM168 precursor RNA does not encode a protein. A nucleotide sequence identical or highly similar to the nucleotide sequence of VGAM168 precursor RNA is designated SEQ ID:154, and is provided hereinbelow with reference to the sequence listing part. Nucleotide sequence SEQ ID:154 is located at position 83136 relative to the genome of Vaccinia Virus.

VGAM168 precursor RNA folds onto itself, forming VGAM168 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM168 folded precursor RNA into VGAM168 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM168 RNA is designated SEQ ID:503, and is provided hereinbelow with reference to the sequence listing part.

VGAM168 host target gene, herein designated VGAM HOST TARGET GENE, encodes a corresponding messenger RNA, VGAM168 host target RNA, herein designated VGAM HOST TARGET RNA. VGAM168 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM168 RNA, herein designated VGAM RNA, binds complementarily to one or more host target binding sites located in untranslated regions of VGAM168 host target RNA, herein designated VGAM HOST TARGET RNA. This complementary binding is due to the fact that the nucleotide sequence of VGAM168 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM168 RNA, herein designated VGAM RNA, may have a different number of host target binding sites in untranslated regions of a VGAM168 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM168 RNA, herein designated VGAM RNA, to host target binding sites on VGAM168 host target RNA, herein designated VGAM HOST TARGET RNA, such as BINDING SITE I, BINDING SITE II and BINDING SITE III, inhibits translation of VGAM168 host target RNA into VGAM168 host target protein, herein designated VGAM HOST TARGET PROTEIN. VGAM host target protein is therefore outlined by a broken line.

It is appreciated that VGAM168 host target gene, herein designated VGAM HOST TARGET GENE, in fact represents a plurality of VGAM168 host target genes. The mRNA of each one of this plurality of VGAM168 host target genes comprises one or more host target binding sites, each having a nucleotide sequence which is at least partly complementary to VGAM168 RNA, herein designated VGAM RNA, and which when bound by VGAM168 RNA causes inhibition of translation of respective one or more VGAM168 host target proteins.

It is further appreciated by one skilled in the art that the mode of translational inhibition illustrated by FIG. 1 with specific reference to translational inhibition exerted by VGAM168 gene, herein designated VGAM GENE, on one or more VGAM168 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM168 is inhibition of expression of host target genes, as part of a novel viral mechanism of attacking a host. Accordingly, utilities of VGAM168 include diagnosis, prevention and treatment of viral infection by Vaccinia Virus. Specific functions, and accordingly utilities, of VGAM168 correlate with, and may be deduced from, the identity of the host target genes which VGAM168 binds and inhibits, and the function of these host target genes, as elaborated hereinbelow.

Nucleotide sequences of the VGAM168 precursor RNA, herein designated VGAM PRECURSOR RNA, and of the ‘diced’ VGAM168 RNA, herein designated VGAM RNA, and a schematic representation of the secondary folding of VGAM168 folded precursor RNA, herein designated VGAM FOLDED PRECURSOR RNA, of VGAM168 are further described hereinbelow with reference to Table 1.

Nucleotide sequences of host target binding sites, such as BINDING SITE-I, BINDING SITE-II and BINDING SITE-III of FIG. 1, found on VGAM168 host target RNA, and schematic representation of the complementarity of each of these host target binding sites to VGAM168 RNA, herein designated VGAM RNA, are described hereinbelow with reference to Table 2.

As mentioned hereinabove with reference to FIG. 1, a function of VGAM168 gene, herein designated VGAM is inhibition of expression of VGAM168 target genes. It is appreciated that specific functions, and accordingly utilities, of VGAM168 correlate with, and may be deduced from, the identity of the target genes which VGAM168 binds and inhibits, and the function of these target genes, as elaborated hereinbelow.

BTB and CNC Homology 1, Basic Leucine Zipper Transcription Factor 2 (BACH2, Accession NM_{—}021813) is a VGAM168 host target gene. BACH2 BINDING SITE is HOST TARGET binding site found in the 3′ untranslated region of mRNA encoded by BACH2, corresponding to a HOST TARGET binding site such as BINDING SITE I, BINDING SITE II or BINDING SITE III. Table 2 illustrates the complementarity of the nucleotide sequences of BACH2 BINDING SITE, designated SEQ ID:1961, to the nucleotide sequence of VGAM168 RNA, herein designated VGAM RNA, also designated SEQ ID:503.

A function of VGAM168 is therefore inhibition of BTB and CNC Homology 1, Basic Leucine Zipper Transcription Factor 2 (BACH2, Accession NM_{—}021813), a gene which acts as repressor or activator, binds to maf recognition elements. Accordingly, utilities of VGAM168 include diagnosis, prevention and treatment of diseases and clinical conditions associated with BACH2. The function of BACH2 and its association with various diseases and clinical conditions, has been established by previous studies, as described hereinabove with reference to VGAM119. Giant Axonal Neuropathy (gigaxonin) (GAN, Accession NM_{—}022041) is another VGAM168 host target gene. GAN BINDING SITE is HOST TARGET binding site found in the 3′ untranslated region of mRNA encoded by GAN, corresponding to a HOST TARGET binding site such as BINDING SITE I, BINDING SITE II or BINDING SITE III. Table 2 illustrates the complementarity of the nucleotide sequences of GAN BINDING SITE, designated SEQ ID:1974, to the nucleotide sequence of VGAM168 RNA, herein designated VGAM RNA, also designated SEQ ID:503.

Another function of VGAM168 is therefore inhibition of Giant Axonal Neuropathy (gigaxonin) (GAN, Accession NM_{—}022041), a gene which plays an important role in neurofilament architecture. Accordingly, utilities of VGAM168 include diagnosis, prevention and treatment of diseases and clinical conditions associated with GAN. The function of GAN and its association with various diseases and clinical conditions, has been established by previous studies, as described hereinabove with reference to VGAM67. MHC Class II Transactivator (MHC2TA, Accession NM_{—}000246) is another VGAM168 host target gene. MHC2TA BINDING SITE is HOST TARGET binding site found in the 3′ untranslated region of mRNA encoded by MHC2TA, corresponding to a HOST TARGET binding site such as BINDING SITE I, BINDING SITE II or BINDING SITE III. Table 2 illustrates the complementarity of the nucleotide sequences of MHC2TA BINDING SITE, designated SEQ ID:720, to the nucleotide sequence of VGAM168 RNA, herein designated VGAM RNA, also designated SEQ ID:503.

Another function of VGAM168 is therefore inhibition of MHC Class II Transactivator (MHC2TA, Accession NM_{—}000246). Accordingly, utilities of VGAM168 include diagnosis, prevention and treatment of diseases and clinical conditions associated with MHC2TA. Myotubular Myopathy 1 (MTM1, Accession NM_{—}000252) is another VGAM168 host target gene. MTM1 BINDING SITE is HOST TARGET binding site found in the 3′ untranslated region of mRNA encoded by MTM1, corresponding to a HOST TARGET binding site such as BINDING SITE I, BINDING SITE II or BINDING SITE III. Table 2 illustrates the complementarity of the nucleotide sequences of MTM1 BINDING SITE, designated SEQ ID:723, to the nucleotide sequence of VGAM168 RNA, herein designated VGAM RNA, also designated SEQ ID:503.

Another function of VGAM168 is therefore inhibition of Myotubular Myopathy 1 (MTM1, Accession NM_{—}000252). Accordingly, utilities of VGAM168 include diagnosis, prevention and treatment of diseases and clinical conditions associated with MTM1. AWP1 (Accession NM_{—}019006) is another VGAM168 host target gene. AWP1 BINDING SITE is HOST TARGET binding site found in the 3′ untranslated region of mRNA encoded by AWP1, corresponding to a HOST TARGET binding site such as BINDING SITE I, BINDING SITE II or BINDING SITE III. Table 2 illustrates the complementarity of the nucleotide sequences of AWP1 BINDING SITE, designated SEQ ID:1872, to the nucleotide sequence of VGAM168 RNA, herein designated VGAM RNA, also designated SEQ ID:503.

Another function of VGAM168 is therefore inhibition of AWP1 (Accession NM_{—}019006). Accordingly, utilities of VGAM168 include diagnosis, prevention and treatment of diseases and clinical conditions associated with AWP1. Bladder Cancer Associated Protein (BLCAP, Accession NM_{—}006698) is another VGAM168 host target gene. BLCAP BINDING SITE is HOST TARGET binding site found in the 3′ untranslated region of mRNA encoded by BLCAP, corresponding to a HOST TARGET binding site such as BINDING SITE I, BINDING SITE II or BINDING SITE III. Table 2 illustrates the complementarity of the nucleotide sequences of BLCAP BINDING SITE, designated SEQ ID:1324, to the nucleotide sequence of VGAM168 RNA, herein designated VGAM RNA, also designated SEQ ID:503.

Another function of VGAM168 is therefore inhibition of Bladder Cancer Associated Protein (BLCAP, Accession NM_{—}006698). Accordingly, utilities of VGAM168 include diagnosis, prevention and treatment of diseases and clinical conditions associated with BLCAP. Chromosome 5 Open Reading Frame 3 (C5orf3, Accession NM_{—}018691) is another VGAM168 host target gene. C5orf3 BINDING SITE is HOST TARGET binding site found in the 3′ untranslated region of mRNA encoded by C5orf3, corresponding to a HOST TARGET binding site such as BINDING SITE I, BINDING SITE II or BINDING SITE III. Table 2 illustrates the complementarity of the nucleotide sequences of C5orf3 BINDING SITE, designated SEQ ID:1859, to the nucleotide sequence of VGAM168 RNA, herein designated VGAM RNA, also designated SEQ ID:503.

Another function of VGAM168 is therefore inhibition of Chromosome 5 Open Reading Frame 3 (C5orf3, Accession NM_{—}018691). Accordingly, utilities of VGAM168 include diagnosis, prevention and treatment of diseases and clinical conditions associated with C5orf3. CRK7 (Accession NM_{—}016507) is another VGAM168 host target gene. CRK7 BINDING SITE is HOST TARGET binding site found in the 3′ untranslated region of mRNA encoded by CRK7, corresponding to a HOST TARGET binding site such as BINDING SITE I, BINDING SITE II or BINDING SITE III. Table 2 illustrates the complementarity of the nucleotide sequences of CRK7 BINDING SITE, designated SEQ ID: 1690, to the nucleotide sequence of VGAM168 RNA, herein designated VGAM RNA, also designated SEQ ID:503.

Another function of VGAM168 is therefore inhibition of CRK7 (Accession NM_{—}016507). Accordingly, utilities of VGAM168 include diagnosis, prevention and treatment of diseases and clinical conditions associated with CRK7. FLJ11101 (Accession NM_{—}018322) is another VGAM168 host target gene. FLJ11101 BINDING SITE is HOST TARGET binding site found in the 3′ untranslated region of mRNA encoded by FLJ11101, corresponding to a HOST TARGET binding site such as BINDING SITE I, BINDING SITE II or BINDING SITE III. Table 2 illustrates the complementarity of the nucleotide sequences of FLJ11101 BINDING SITE, designated SEQ ID:1814, to the nucleotide sequence of VGAM168 RNA, herein designated VGAM RNA, also designated SEQ ID:503.

Another function of VGAM168 is therefore inhibition of FLJ11101 (Accession NM_{—}018322). Accordingly, utilities of VGAM168 include diagnosis, prevention and treatment of diseases and clinical conditions associated with FLJ11101. FLJ12876 (Accession NM_{—}022754) is another VGAM168 host target gene. FLJ12876 BINDING SITE is HOST TARGET binding site found in the 3′ untranslated region of mRNA encoded by FLJ12876, corresponding to a HOST TARGET binding site such as BINDING SITE I, BINDING SITE II or BINDING SITE III. Table 2 illustrates the complementarity of the nucleotide sequences of FLJ12876 BINDING SITE, designated SEQ ID:2004, to the nucleotide sequence of VGAM168 RNA, herein designated VGAM RNA, also designated SEQ ID:503.

Another function of VGAM168 is therefore inhibition of FLJ12876 (Accession NM_{—}022754). Accordingly, utilities of VGAM168 include diagnosis, prevention and treatment of diseases and clinical conditions associated with FLJ12876. Kruppel-like Factor 12 (KLF12, Accession NM_{—}007249) is another VGAM168 host target gene. KLF12 BINDING SITE is HOST TARGET binding site found in the 3′ untranslated region of mRNA encoded by KLF12, corresponding to a HOST TARGET binding site such as BINDING SITE I, BINDING SITE II or BINDING SITE III. Table 2 illustrates the complementarity of the nucleotide sequences of KLF12 BINDING SITE, designated SEQ ID:1374, to the nucleotide sequence of VGAM168 RNA, herein designated VGAM RNA, also designated SEQ ID:503.

Another function of VGAM168 is therefore inhibition of Kruppel-like Factor 12 (KLF12, Accession NM_{—}007249). Accordingly, utilities of VGAM168 include diagnosis, prevention and treatment of diseases and clinical conditions associated with KLF12. MGC4734 (Accession NM_{—}145051) is another VGAM168 host target gene. MGC4734 BINDING SITE is HOST TARGET binding site found in the 3′ untranslated region of mRNA encoded by MGC4734, corresponding to a HOST TARGET binding site such as BINDING SITE I, BINDING SITE II or BINDING SITE III. Table 2 illustrates the complementarity of the nucleotide sequences of MGC4734 BINDING SITE, designated SEQ ID:2513, to the nucleotide sequence of VGAM168 RNA, herein designated VGAM RNA, also designated SEQ ID:503.

Another function of VGAM168 is therefore inhibition of MGC4734 (Accession NM_{—}145051). Accordingly, utilities of VGAM168 include diagnosis, prevention and treatment of diseases and clinical conditions associated with MGC4734. PRO2000 (Accession NM_{—}014109) is another VGAM168 host target gene. PRO2000 BINDING SITE is HOST TARGET binding site found in the 3′ untranslated region of mRNA encoded by PRO2000, corresponding to a HOST TARGET binding site such as BINDING SITE I, BINDING SITE II or BINDING SITE III. Table 2 illustrates the complementarity of the nucleotide sequences of PRO2000 BINDING SITE, designated SEQ ID: 1469, to the nucleotide sequence of VGAM168 RNA, herein designated VGAM RNA, also designated SEQ ID:503.

Another function of VGAM168 is therefore inhibition of PRO2000 (Accession NM_{—}014109). Accordingly, utilities of VGAM168 include diagnosis, prevention and treatment of diseases and clinical conditions associated with PRO2000. RAB33B, Member RAS Oncogene Family (RAB33B, Accession NM_{—}031296) is another VGAM168 host target gene. RAB33B BINDING SITE is HOST TARGET binding site found in the 3′ untranslated region of mRNA encoded by RAB33B, corresponding to a HOST TARGET binding site such as BINDING SITE I, BINDING SITE II or BINDING SITE III. Table 2 illustrates the complementarity of the nucleotide sequences of RAB33B BINDING SITE, designated SEQ ID:2191, to the nucleotide sequence of VGAM168 RNA, herein designated VGAM RNA, also designated SEQ ID:503.

Another function of VGAM168 is therefore inhibition of RAB33B, Member RAS Oncogene Family (RAB33B, Accession NM_{—}031296). Accordingly, utilities of VGAM168 include diagnosis, prevention and treatment of diseases and clinical conditions associated with RAB33B. Regulator of G-protein Signalling 12 (RGS12, Accession NM_{—}002926) is another VGAM168 host target gene. RGS12 BINDING SITE is HOST TARGET binding site found in the 3′ untranslated region of mRNA encoded by RGS12, corresponding to a HOST TARGET binding site such as BINDING SITE I, BINDING SITE II or BINDING SITE III. Table 2 illustrates the complementarity of the nucleotide sequences of RGS12 BINDING SITE, designated SEQ ID:972, to the nucleotide sequence of VGAM168 RNA, herein designated VGAM RNA, also designated SEQ ID:503.

Another function of VGAM168 is therefore inhibition of Regulator of G-protein Signalling 12 (RGS12, Accession NM_{—}002926). Accordingly, utilities of VGAM168 include diagnosis, prevention and treatment of diseases and clinical conditions associated with RGS12. Zinc Finger Protein 387 (ZNF387, Accession NM_{—}014682) is another VGAM168 host target gene. ZNF387 BINDING SITE is HOST TARGET binding site found in the 3′ untranslated region of mRNA encoded by ZNF387, corresponding to a HOST TARGET binding site such as BINDING SITE I, BINDING SITE II or BINDING SITE III. Table 2 illustrates the complementarity of the nucleotide sequences of ZNF387 BINDING SITE, designated SEQ ID:1522, to the nucleotide sequence of VGAM168 RNA, herein designated VGAM RNA, also designated SEQ ID:503.

Another function of VGAM168 is therefore inhibition of Zinc Finger Protein 387 (ZNF387, Accession NM_{—}014682). Accordingly, utilities of VGAM168 include diagnosis, prevention and treatment of diseases and clinical conditions associated with ZNF387. LOC158381 (Accession XM_{—}048461) is another VGAM168 host target gene. LOC158381 BINDING SITE is HOST TARGET binding site found in the 3′ untranslated region of mRNA encoded by LOC158381, corresponding to a HOST TARGET binding site such as BINDING SITE I, BINDING SITE II or BINDING SITE III. Table 2 illustrates the complementarity of the nucleotide sequences of LOC158381 BINDING SITE, designated SEQ ID:2905, to the nucleotide sequence of VGAM168 RNA, herein designated VGAM RNA, also designated SEQ ID:503.

Another function of VGAM168 is therefore inhibition of LOC158381 (Accession XM_{—}048461). Accordingly, utilities of VGAM168 include diagnosis, prevention and treatment of diseases and clinical conditions associated with LOC158381. LOC196411 (Accession XM_{—}113714) is another VGAM168 host target gene. LOC196411 BINDING SITE is HOST TARGET binding site found in the 3′ untranslated region of mRNA encoded by LOC196411, corresponding to a HOST TARGET binding site such as BINDING SITE I, BINDING SITE II or BINDING SITE III. Table 2 illustrates the complementarity of the nucleotide sequences of LOC196411 BINDING SITE, designated SEQ ID:3412, to the nucleotide sequence of VGAM168 RNA, herein designated VGAM RNA, also designated SEQ ID:503.

Another function of VGAM168 is therefore inhibition of LOC196411 (Accession XM_{—}113714). Accordingly, utilities of VGAM168 include diagnosis, prevention and treatment of diseases and clinical conditions associated with LOC196411. LOC51186 (Accession NM_{—}016303) is another VGAM168 host target gene. LOC51186 BINDING SITE is HOST TARGET binding site found in the 5′ untranslated region of mRNA encoded by LOC51186, corresponding to a HOST TARGET binding site such as BINDING SITE I, BINDING SITE II or BINDING SITE III. Table 2 illustrates the complementarity of the nucleotide sequences of LOC51186 BINDING SITE, designated SEQ ID:1680, to the nucleotide sequence of VGAM168 RNA, herein designated VGAM RNA, also designated SEQ ID:503.

Another function of VGAM168 is therefore inhibition of LOC51186 (Accession NM_{—}016303). Accordingly, utilities of VGAM168 include diagnosis, prevention and treatment of diseases and clinical conditions associated with LOC51186. FIG. 1 further provides a conceptual description of a novel bioinformatically detected viral gene of the present invention, referred to here as Viral Genomic Address Messenger 169 (VGAM169) viral gene, which modulates expression of respective host target genes thereof, the function and utility of which host target genes is known in the art.

VGAM169 is a novel bioinformatically detected regulatory, non protein coding, viral micro RNA (miRNA) gene. The method by which VGAM169 was detected is described hereinabove with reference to FIGS. 1-8.

VGAM169 gene, herein designated VGAM GENE, is a viral gene contained in the genome of Vaccinia Virus. VGAM169 host target gene, herein designated VGAM HOST TARGET GENE, is a human gene contained in the human genome.

VGAM169 gene encodes a VGAM169 precursor RNA, herein designated VGAM PRECURSOR RNA. Similar to other miRNA genes, and unlike most ordinary genes, VGAM169 precursor RNA does not encode a protein. A nucleotide sequence identical or highly similar to the nucleotide sequence of VGAM169 precursor RNA is designated SEQ ID:155, and is provided hereinbelow with reference to the sequence listing part. Nucleotide sequence SEQ ID:155 is located at position 87488 relative to the genome of Vaccinia Virus.

VGAM169 precursor RNA folds onto itself, forming VGAM169 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM169 folded precursor RNA into VGAM169 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM169 RNA is designated SEQ ID:504, and is provided hereinbelow with reference to the sequence listing part.

VGAM169 host target gene, herein designated VGAM HOST TARGET GENE, encodes a corresponding messenger RNA, VGAM169 host target RNA, herein designated VGAM HOST TARGET RNA. VGAM169 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM169 RNA, herein designated VGAM RNA, binds complementarily to one or more host target binding sites located in untranslated regions of VGAM169 host target RNA, herein designated VGAM HOST TARGET RNA. This complementary binding is due to the fact that the nucleotide sequence of VGAM169 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM169 RNA, herein designated VGAM RNA, may have a different number of host target binding sites in untranslated regions of a VGAM169 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM169 RNA, herein designated VGAM RNA, to host target binding sites on VGAM169 host target RNA, herein designated VGAM HOST TARGET RNA, such as BINDING SITE I, BINDING SITE II and BINDING SITE III, inhibits translation of VGAM169 host target RNA into VGAM169 host target protein, herein designated VGAM HOST TARGET PROTEIN. VGAM host target protein is therefore outlined by a broken line.

It is appreciated that VGAM169 host target gene, herein designated VGAM HOST TARGET GENE, in fact represents a plurality of VGAM169 host target genes. The mRNA of each one of this plurality of VGAM169 host target genes comprises one or more host target binding sites, each having a nucleotide sequence which is at least partly complementary to VGAM169 RNA, herein designated VGAM RNA, and which when bound by VGAM169 RNA causes inhibition of translation of respective one or more VGAM169 host target proteins.

It is further appreciated by one skilled in the art that the mode of translational inhibition illustrated by FIG. 1 with specific reference to translational inhibition exerted by VGAM169 gene, herein designated VGAM GENE, on one or more VGAM169 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM169 is inhibition of expression of host target genes, as part of a novel viral mechanism of attacking a host. Accordingly, utilities of VGAM169 include diagnosis, prevention and treatment of viral infection by Vaccinia Virus. Specific functions, and accordingly utilities, of VGAM169 correlate with, and may be deduced from, the identity of the host target genes which VGAM169 binds and inhibits, and the function of these host target genes, as elaborated hereinbelow.

Nucleotide sequences of the VGAM169 precursor RNA, herein designated VGAM PRECURSOR RNA, and of the ‘diced’ VGAM169 RNA, herein designated VGAM RNA, and a schematic representation of the secondary folding of VGAM169 folded precursor RNA, herein designated VGAM FOLDED PRECURSOR RNA, of VGAM169 are further described hereinbelow with reference to Table 1.

Nucleotide sequences of host target binding sites, such as BINDING SITE-I, BINDING SITE-II and BINDING SITE-III of FIG. 1, found on VGAM169 host target RNA, and schematic representation of the complementarity of each of these host target binding sites to VGAM169 RNA, herein designated VGAM RNA, are described hereinbelow with reference to Table 2.

As mentioned hereinabove with reference to FIG. 1, a function of VGAM169 gene, herein designated VGAM is inhibition of expression of VGAM169 target genes. It is appreciated that specific functions, and accordingly utilities, of VGAM169 correlate with, and may be deduced from, the identity of the target genes which VGAM169 binds and inhibits, and the function of these target genes, as elaborated hereinbelow.

Radixin (RDX, Accession NM_{—}002906) is a VGAM169 host target gene. RDX BINDING SITE is HOST TARGET binding site found in the 3′ untranslated region of mRNA encoded by RDX, corresponding to a HOST TARGET binding site such as BINDING SITE I, BINDING SITE II or BINDING SITE III. Table 2 illustrates the complementarity of the nucleotide sequences of RDX BINDING SITE, designated SEQ ID:971, to the nucleotide sequence of VGAM169 RNA, herein designated VGAM RNA, also designated SEQ ID:504.

A function of VGAM169 is therefore inhibition of Radixin (RDX, Accession NM_{—}002906), a gene which plays a crucial role in the binding of the barbed end of actin filaments to the plasma membrane. Accordingly, utilities of VGAM169 include diagnosis, prevention and treatment of diseases and clinical conditions associated with RDX. The function of RDX has been established by previous studies. Radixin is a cytoskeletal protein that may be important in linking actin to the plasma membrane. Cloning of the murine and porcine radixin cDNAs demonstrated a protein highly homologous to ezrin (OMIM Ref. No. 123900) and moesin (OMIM Ref. No. 309845). Wilgenbus et al. (1993) cloned and sequenced the human radixin cDNA and found the predicted amino acid sequence for the human protein to be nearly identical to those predicted for radixin in the two other species. Animal model experiments lend further support to the function of RDX. The ezrin-radixin-moesin (ERM) family of proteins crosslink actin filaments and integral membrane proteins. Radixin (encoded by Rdx) is the dominant ERM protein in the liver of wildtype mice and is concentrated at bile canalicular membranes (BCM). Kikuchi et al. (2002) showed that Rdx −/− mice are normal at birth, but their serum concentrations of conjugated bilirubin begin to increase gradually around 4 weeks of age, and they show mild liver injury after 8 weeks. This phenotype is similar to human conjugated hyperbilirubinemia in Dubin-Johnson syndrome (OMIM Ref. No. 237500), which is caused by mutations in the ABCC2 gene (OMIM Ref. No. 601107), although Dubin-Johnson syndrome is not associated with overt liver injury. In wildtype mice, the protein product of the ABCC2 gene, multidrug resistance protein-2, or MRP2, concentrates at BCMs to secrete conjugated bilirubin into bile. In the BCMs of Rdx −/− mice, Mrp2 is decreased compared with other BCM proteins such as dipeptidyl peptidase IV (CD26; 102720) and P-glycoproteins. In vitro binding studies showed that radixin associates directly with the carboxy-terminal cytoplasmic domain of human MRP2. These findings indicated that radixin is required for secretion of conjugated bilirubin through its support of Mrp2 localization at BCMs.

It is appreciated that the abovementioned animal model for RDX is acknowledged by those skilled in the art as a scientifically valid animal model, as can be further appreciated from the publications sited hereinbelow.

Full details of the abovementioned studies are described in the following publications, the disclosure of which are hereby incorporated by reference:
Wilgenbus, K. K.; Milatovich, A.; Francke, U.; Furthmayr, H.: Molecular cloning, cDNA sequence, and chromosomal assignment of the human radixin gene and two dispersed pseudogenes. Genomics 16: 199-206, 1993.; and
Kikuchi, S.; Hata, M.; Fukumoto, K.; Yamane, Y.; Matsui, T.; Tamura, A.; Yonemura, S.; Yamagishi, H.; Keppler, D.; Tsukita, S.; Tsukita, S.: Radixin deficiency causes conjugated hyperbi.

Further studies establishing the function and utilities of RDX are found in John Hopkins OMIM database record ID 179410, and in sited publications numbered 624-625 listed in the bibliography section hereinbelow, which are also hereby incorporated by reference. Transient Receptor Potential Cation Channel, Sub family C, Member 3 (TRPC3, Accession NM_{—}003305) is another VGAM169 host target gene. TRPC3 BINDING SITE is HOST TARGET binding site found in the 3′ untranslated region of mRNA encoded by TRPC3, corresponding to a HOST TARGET binding site such as BINDING SITE I, BINDING SITE II or BINDING SITE III. Table 2 illustrates the complementarity of the nucleotide sequences of TRPC3 BINDING SITE, designated SEQ ID:1010, to the nucleotide sequence of VGAM169 RNA, herein designated VGAM RNA, also designated SEQ ID:504.

Another function of VGAM169 is therefore inhibition of Transient Receptor Potential Cation Channel, Sub family C, Member 3 (TRPC3, Accession NM_{—}003305). Accordingly, utilities of VGAM169 include diagnosis, prevention and treatment of diseases and clinical conditions associated with TRPC3. LOC131000 (Accession XM_{—}067145) is another VGAM169 host target gene. LOC131000 BINDING SITE is HOST TARGET binding site found in the 3′ untranslated region of mRNA encoded by LOC131000, corresponding to a HOST TARGET binding site such as BINDING SITE I, BINDING SITE II or BINDING SITE III. Table 2 illustrates the complementarity of the nucleotide sequences of LOC131000 BINDING SITE, designated SEQ ID:3038, to the nucleotide sequence of VGAM169 RNA, herein designated VGAM RNA, also designated SEQ ID:504.

Another function of VGAM169 is therefore inhibition of LOC131000 (Accession XM_{—}067145). Accordingly, utilities of VGAM169 include diagnosis, prevention and treatment of diseases and clinical conditions associated with LOC131000. FIG. 1 further provides a conceptual description of a novel bioinformatically detected viral gene of the present invention, referred to here as Viral Genomic Address Messenger 170 (VGAM170) viral gene, which modulates expression of respective host target genes thereof, the function and utility of which host target genes is known in the art.

VGAM170 is a novel bioinformatically detected regulatory, non protein coding, viral micro RNA (miRNA) gene. The method by which VGAM170 was detected is described hereinabove with reference to FIGS. 1-8.

VGAM170 gene, herein designated VGAM GENE, is a viral gene contained in the genome of Vaccinia Virus. VGAM170 host target gene, herein designated VGAM HOST TARGET GENE, is a human gene contained in the human genome.

VGAM170 gene encodes a VGAM170 precursor RNA, herein designated VGAM PRECURSOR RNA. Similar to other miRNA genes, and unlike most ordinary genes, VGAM170 precursor RNA does not encode a protein. A nucleotide sequence identical or highly similar to the nucleotide sequence of VGAM170 precursor RNA is designated SEQ ID:156, and is provided hereinbelow with reference to the sequence listing part. Nucleotide sequence SEQ ID:156 is located at position 88940 relative to the genome of Vaccinia Virus.

VGAM170 precursor RNA folds onto itself, forming VGAM170 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM170 folded precursor RNA into VGAM170 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM170 RNA is designated SEQ ID:505, and is provided hereinbelow with reference to the sequence listing part.

VGAM170 host target gene, herein designated VGAM HOST TARGET GENE, encodes a corresponding messenger RNA, VGAM170 host target RNA, herein designated VGAM HOST TARGET RNA. VGAM170 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM170 RNA, herein designated VGAM RNA, binds complementarily to one or more host target binding sites located in untranslated regions of VGAM170 host target RNA, herein designated VGAM HOST TARGET RNA. This complementary binding is due to the fact that the nucleotide sequence of VGAM170 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM170 RNA, herein designated VGAM RNA, may have a different number of host target binding sites in untranslated regions of a VGAM170 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM170 RNA, herein designated VGAM RNA, to host target binding sites on VGAM170 host target RNA, herein designated VGAM HOST TARGET RNA, such as BINDING SITE I, BINDING SITE II and BINDING SITE III, inhibits translation of VGAM170 host target RNA into VGAM170 host target protein, herein designated VGAM HOST TARGET PROTEIN. VGAM host target protein is therefore outlined by a broken line.

It is appreciated that VGAM170 host target gene, herein designated VGAM HOST TARGET GENE, in fact represents a plurality of VGAM170 host target genes. The mRNA of each one of this plurality of VGAM170 host target genes comprises one or more host target binding sites, each having a nucleotide sequence which is at least partly complementary to VGAM170 RNA, herein designated VGAM RNA, and which when bound by VGAM170 RNA causes inhibition of translation of respective one or more VGAM170 host target proteins.

It is further appreciated by one skilled in the art that the mode of translational inhibition illustrated by FIG. 1 with specific reference to translational inhibition exerted by VGAM170 gene, herein designated VGAM GENE, on one or more VGAM170 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM170 is inhibition of expression of host target genes, as part of a novel viral mechanism of attacking a host. Accordingly, utilities of VGAM170 include diagnosis, prevention and treatment of viral infection by Vaccinia Virus. Specific functions, and accordingly utilities, of VGAM170 correlate with, and may be deduced from, the identity of the host target genes which VGAM170 binds and inhibits, and the function of these host target genes, as elaborated hereinbelow.

Nucleotide sequences of the VGAM170 precursor RNA, herein designated VGAM PRECURSOR RNA, and of the ‘diced’ VGAM170 RNA, herein designated VGAM RNA, and a schematic representation of the secondary folding of VGAM170 folded precursor RNA, herein designated VGAM FOLDED PRECURSOR RNA, of VGAM170 are further described hereinbelow with reference to Table 1.

Nucleotide sequences of host target binding sites, such as BINDING SITE-I, BINDING SITE-II and BINDING SITE-III of FIG. 1, found on VGAM170 host target RNA, and schematic representation of the complementarity of each of these host target binding sites to VGAM170 RNA, herein designated VGAM RNA, are described hereinbelow with reference to Table 2.

As mentioned hereinabove with reference to FIG. 1, a function of VGAM170 gene, herein designated VGAM is inhibition of expression of VGAM170 target genes. It is appreciated that specific functions, and accordingly utilities, of VGAM170 correlate with, and may be deduced from, the identity of the target genes which VGAM170 binds and inhibits, and the function of these target genes, as elaborated hereinbelow.

Actin, Alpha 2, Smooth Muscle, Aorta (ACTA2, Accession NM_{—}001613) is a VGAM170 host target gene. ACTA2 BINDING SITE is HOST TARGET binding site found in the 3′ untranslated region of mRNA encoded by ACTA2, corresponding to a HOST TARGET binding site such as BINDING SITE I, BINDING SITE II or BINDING SITE III. Table 2 illustrates the complementarity of the nucleotide sequences of ACTA2 BINDING SITE, designated SEQ ID:839, to the nucleotide sequence of VGAM170 RNA, herein designated VGAM RNA, also designated SEQ ID:505.

A function of VGAM170 is therefore inhibition of Actin, Alpha 2, Smooth Muscle, Aorta (ACTA2, Accession NM_{—}001613). Accordingly, utilities of VGAM170 include diagnosis, prevention and treatment of diseases and clinical conditions associated with ACTA2. Aspartate Beta-hydroxylase (ASPH, Accession NM_{—}032466) is another VGAM170 host target gene. ASPH BINDING SITE1 and ASPH BINDING SITE2 are HOST TARGET binding sites found in untranslated regions of mRNA encoded by ASPH, corresponding to HOST TARGET binding sites such as BINDING SITE I, BINDING SITE II or BINDING SITE III. Table 2 illustrates the complementarity of the nucleotide sequences of ASPH BINDING SITE1 and ASPH BINDING SITE2, designated SEQ ID:2251 and SEQ ID:2253 respectively, to the nucleotide sequence of VGAM170 RNA, herein designated VGAM RNA, also designated SEQ ID:505.

Another function of VGAM170 is therefore inhibition of Aspartate Beta-hydroxylase (ASPH, Accession NM_{—}032466), a gene which specifically hydroxylates the beta carbon of aspartic acid or asparagine residues in certain epidermal growth factor (EGF)-like domains of a number of proteins. Accordingly, utilities of VGAM170 include diagnosis, prevention and treatment of diseases and clinical conditions associated with ASPH. The function of ASPH has been established by previous studies. In hepatocellular carcinoma (HCC; 114550), one of the most prevalent tumors in the world which occurs with especially high frequency in sub-Saharan Africa and the Far East, a specific antigen is highly expressed; it is highly expressed also in cholangiocarcinomas. Lavaissiere et al. (1996) reported cDNA cloning of the human gene encoding this antigen, aspartyl(asparaginyl)-beta-hydroxylase (symbolized HAAH by them), and demonstrated that in these tumor lines it is expressed in an enzymatically active form. The gene encodes a deduced 744-amino acid polypeptide with high homology (81%) to the bovine gene (Jia et al., 1992). Lavaissiere et al. (1996) found that their cDNA human sequence was 99% homologous to the sequence for ASPH reported by Korioth et al. (1994), differing only at amino acid residues 565 (tyr to ile), 575 (trp-trp-thr to cys-gly), 585 (asp to gin), and 709 (arg to lys). They noted also a silent TCG-to-TCA transition at peptide residue 161. Lavaissiere et al. (1996) speculated about the possible relationship of the malignant phenotype of regulated aspartyl/asparaginyl-beta-hydroxylation in EGF-like domains of proteins such as the mammalian Notch homologs (e.g., 190198, 600275, and 600276), which are known to be involved in cell differentiation and whose cytoplasmic domains have been shown to be oncogenic. By screening a heart cDNA library, followed by RT-PCR, Lim et al. (2000) isolated cDNAs encoding the 225-amino acid junctin protein and a 210-amino acid isoform. The authors noted that a 73-residue stretch in junctin has a completely matched region in the ASPH protein. Southern blot analysis indicated that junctin and ASPH exist as a single-copy gene. Northern blot analysis revealed expression of 3.0- and 4.2-kb transcripts in cardiac and skeletal muscle; expression was higher in skeletal muscle. SDS-PAGE analysis of the translated cDNAs showed expression of 26- and 28-kD proteins. By screening a skeletal muscle cDNA library with a dog junctin probe, Treves et al. (2000) identified cDNAs encoding human junctin and junctate. Sequence analysis predicted that junctate, a 299-amino acid protein, shares the first 93 amino acids of the long isoform of junctin (and, partially, of ASPH), whereas its 64 C-terminal residues are identical to the central region of ASPH. Northern blot analysis detected a 2.6-kb transcript in heart, brain, pancreas, placenta, lung, liver, kidney, and skeletal muscle; highest levels were in heart, brain, and pancreas, and lowest levels were in skeletal muscle. In contrast, junctin was expressed only in cardiac and skeletal muscle. Southern blot and PCR analyses indicated that ASPH, junctin, and junctate are splice variants of the same gene; ASPH uses exons 1, 3, 5, and 8 through 16, whereas junctin uses exons 2, 3, 5, and 6, and junctate uses exons 2 through 5 and 8 through 16. Fluorescence microscopy showed junctate expression in sarco(endo)plasmic reticulum membranes. Immunoblot analysis indicated that junctate is expressed as a 32-kD protein in kidney microsomes. Binding analysis determined that junctate binds calcium with high capacity and moderate affinity.

Full details of the abovementioned studies are described in the following publications, the disclosure of which are hereby incorporated by reference:
Lavaissiere, L.; Jia, S.; Nishiyama, M.; de la Monte, S.; Stern, A. M.; Wands, J. R.; Friedman, P. A.: Overexpression of human aspartyl(asparaginyl)-beta-hydroxylase in hepatocellular carcinoma and cholangiocarcinoma. J. Clin. Invest. 98: 1313-1323, 1996.; and
Treves, S.; Feriotto, G.; Moccagatta, L.; Gambari, R.; Zorzato, F.: Molecular cloning, expression, functional characterization, chromosomal localization, and gene structure of junctate.

Further studies establishing the function and utilities of ASPH are found in John Hopkins OMIM database record ID 600582, and in sited publications numbered 2321-2327 listed in the bibliography section hereinbelow, which are also hereby incorporated by reference. Cell Division Cycle 42 (GTP binding protein, 25 kDa) (CDC42, Accession NM_{—}001791) is another VGAM170 host target gene. CDC42 BINDING SITE is HOST TARGET binding site found in the 3′ untranslated region of mRNA encoded by CDC42, corresponding to a HOST TARGET binding site such as BINDING SITE I, BINDING SITE II or BINDING SITE III. Table 2 illustrates the complementarity of the nucleotide sequences of CDC42 BINDING SITE, designated SEQ ID:856, to the nucleotide sequence of VGAM170 RNA, herein designated VGAM RNA, also designated SEQ ID:505.

Another function of VGAM170 is therefore inhibition of Cell Division Cycle 42 (GTP binding protein, 25 kDa) (CDC42, Accession NM_{—}001791). Accordingly, utilities of VGAM170 include diagnosis, prevention and treatment of diseases and clinical conditions associated with CDC42. Cytoplasmic Linker Associated Protein 1 (CLASP1, Accession XM_{—}037105) is another VGAM170 host target gene. CLASP1 BINDING SITE is HOST TARGET binding site found in the 3′ untranslated region of mRNA encoded by CLASP1, corresponding to a HOST TARGET binding site such as BINDING SITE I, BINDING SITE II or BINDING SITE III. Table 2 illustrates the complementarity of the nucleotide sequences of CLASP1 BINDING SITE, designated SEQ ID:2718, to the nucleotide sequence of VGAM170 RNA, herein designated VGAM RNA, also designated SEQ ID:505.

Another function of VGAM170 is therefore inhibition of Cytoplasmic Linker Associated Protein 1 (CLASP1, Accession XM_{—}037105), a gene which plays a role in the local regulation of microtubule dynamics. Accordingly, utilities of VGAM170 include diagnosis, prevention and treatment of diseases and clinical conditions associated with CLASP1. The function of CLASP1 has been established by previous studies. CLIP170 (OMIM Ref. No. 179838) and CLIP115 (OMIM Ref. No. 603432) are cytoplasmic linker proteins that associate specifically with the ends of growing microtubules and may act as anticatastrophe factors. Using a yeast 2-hybrid screen with an N-terminal region of CLIP115 as bait, followed by cDNA library screening, RACE analysis, and EST database searching, Akhmanova et al. (2001) identified mouse and human cDNAs encoding 2 CLIP-associated proteins, CLASP1 and CLASP2 (OMIM Ref. No. 605853). The CLASPs are homologous to a Drosophila microtubule-associated protein termed Orbit or Mast. CLASP1 is identical to the protein encoded by a partial cDNA, KIAA0622, identified by Ishikawa et al. (1998), although the KIAA0622 protein lacks the N-terminal 249 amino acids of the 1,538-amino acid CLASP1 protein reported by Akhmanova et al. (2001). CLASP2 shares approximately 75% identity with the KIAA0627 protein, which is encoded by a partial cDNA also identified by Ishikawa et al. (1998). There are several CLASP isoforms due to alternative splicing. Northern blot analysis of mouse tissues detected highest expression of Clasp1 in brain, heart, and testis, while Clasp2 mRNAs were enriched in the brain. The Clasp2-beta transcript appeared to be brain specific. By RT-PCR analysis, Ishikawa et al. (1998) detected ubiquitous expression of CLASP1, which they called KIAA0622. Akhmanova et al. (2001) showed that CLASPs bind CLIPs and microtubules, colocalize with the CLIPs at microtubule distal ends, and have microtubule-stabilizing effects in transfected cells. After serum induction, CLASPs relocalize to distal segments of microtubules at the leading edge of motile fibroblasts. Akhmanova et al. (2001) provided evidence that this asymmetric CLASP distribution is mediated by phosphatidylinositol 3-kinase (see OMIM Ref. No. 171834) and glycogen synthase kinase 3-beta (OMIM Ref. No. 605004). Antibody injections suggested that CLASP2 is required for the orientation of stabilized microtubules toward the leading edge. The authors proposed that CLASPs are involved in the local regulation of microtubule dynamics in response to positional cues.

Full details of the abovementioned studies are described in the following publications, the disclosure of which are hereby incorporated by reference:
Akhmanova, A.; Hoogenraad, C. C.; Drabek, K.; Stepanova, T.; Dortland, B.; Verkerk, T.; Vermeulen, W.; Burgering, B. M.; De Zeeuw, C. I.; Grosveld, F.; Galjart, N.: CLASPs are CLIP-115 and -170 associating proteins involved in the regional regulation of microtubule dynamics in motile fibroblasts. Cell 104: 923-935, 2001.; and
Ishikawa, K.; Nagase, T.; Suyama, M.; Miyajima, N.; Tanaka, A.; Kotani, H.; Nomura, N.; Ohara, O.: Prediction of the coding sequences of unidentified human genes. X. The complete sequ.

Further studies establishing the function and utilities of CLASP1 are found in John Hopkins OMIM database record ID 605852, and in sited publications numbered 153 and 2136 listed in the bibliography section hereinbelow, which are also hereby incorporated by reference. Copine III (CPNE3, Accession NM_{—}003909) is another VGAM170 host target gene. CPNE3 BINDING SITE is HOST TARGET binding site found in the 3′ untranslated region of mRNA encoded by CPNE3, corresponding to a HOST TARGET binding site such as BINDING SITE I, BINDING SITE II or BINDING SITE III. Table 2 illustrates the complementarity of the nucleotide sequences of CPNE3 BINDING SITE, designated SEQ ID: 1069, to the nucleotide sequence of VGAM170 RNA, herein designated VGAM RNA, also designated SEQ ID:505.

Another function of VGAM170 is therefore inhibition of Copine III (CPNE3, Accession NM_{—}003909), a gene which may function in membrane traffiking. Accordingly, utilities of VGAM170 include diagnosis, prevention and treatment of diseases and clinical conditions associated with CPNE3. The function of CPNE3 has been established by previous studies. By screening human brain cDNAs for the potential to encode proteins larger than 50 kD, Ishikawa et al. (1998) identified a CPNE3 cDNA, which they called KIAA0636. The deduced 537-amino acid CPNE3 protein is 65.7% identical to CPNE1. By SDS-PAGE, the in vitro transcribed/translated product of the CPNE3 cDNA had a molecular mass of 65 kD. RT-PCR detected CPNE3 expression in all human tissues examined By immunoprecipitation and kinase assays, Caudell et al. (2000) serendipitously identified a 60-kD protein identical to CPNE3. CPNE3 contains 2 N-terminal C2 domains, like CPNE1, CPNE6 (OMIM Ref. No. 605688), and CPNE7 (OMIM Ref. No. 605689), but these 4 copines have divergent C termini. CPNE3 is 63%, 52%, and 47% identical to CPNE1, CPNE6, and CPNE7, respectively. Northern blot analysis revealed ubiquitous expression of a 5.0-kb transcript. Biochemical analysis showed that CPNE3 appears to possess endogenous kinase activity, although it lacks a classic kinase domain. CPNE3 is phosphorylated on both serine and threonine residues but not on tyrosine residues.

Full details of the abovementioned studies are described in the following publications, the disclosure of which are hereby incorporated by reference:
Creutz, C. E.; Tomsig, J. L.; Snyder, S. L.; Gautier, M.-C.; Skouri, F.; Beisson, J.; Cohen, J.: The copines, a novel class of C2 domain-containing, calcium-dependent, phospholipid-binding proteins conserved from Paramecium to humans. J. Biol. Chem. 273: 1393-1402, 1998.; and
Caudell, E. G.; Caudell, J. J.; Tang, C.-H.; Yu, T.-K.; Frederick, M. J.; Grimm, E. A.: Characterization of human copine III as a phosphoprotein with associated kinase activity. Biochem.

Further studies establishing the function and utilities of CPNE3 are found in John Hopkins OMIM database record ID 604207, and in sited publications numbered 106 and 2136 listed in the bibliography section hereinbelow, which are also hereby incorporated by reference. Dachshund Homolog (Drosophila) (DACH, Accession NM_{—}080759) is another VGAM170 host target gene. DACH BINDING SITE is HOST TARGET binding site found in the 3′ untranslated region of mRNA encoded by DACH, corresponding to a HOST TARGET binding site such as BINDING SITE I, BINDING SITE II or BINDING SITE III. Table 2 illustrates the complementarity of the nucleotide sequences of DACH BINDING SITE, designated SEQ ID:2388, to the nucleotide sequence of VGAM170 RNA, herein designated VGAM RNA, also designated SEQ ID:505.

Another function of VGAM170 is therefore inhibition of Dachshund Homolog (Drosophila) (DACH, Accession NM_{—}080759), a gene which regulates early progenitor cell proliferation during retinogenesis and pituitary development. Accordingly, utilities of VGAM170 include diagnosis, prevention and treatment of diseases and clinical conditions associated with DACH. The function of DACH has been established by previous studies. The Drosophila ‘dachshund’ (dac) gene is involved in both eye and leg development. Dac, ‘eyeless’ (ey), and ‘eyes absent’ (eya) are considered potential master genes in eye formation. By searching an EST database, Hammond et al. (1998) identified cDNAs corresponding to DACH, a human dac homolog. The authors used DACH cDNAs as a probe to isolate mouse Dach cDNAs from an embryonic library. Both mouse and human Dach mRNAs contain long stretches of trinucleotide repeats in their 5-prime untranslated regions. The predicted 517-amino acid human DACH protein is 99% identical to Dach. Sequence analysis revealed that Drosophila dac and mammalian DACH proteins share conserved domains at their N and C termini, designated Dachbox-N and Dachbox-C, respectively. The SKI (OMIM Ref. No. 164780) protooncogene and the related SNO (OMIM Ref. No. 165340) protein also contain Dachbox-N, as well as a C-terminal motif corresponding to the helical coiled-coil domain beginning in Dachbox-C. While the C-terminal motif is only weakly conserved between SKI and SNO and the dac-related proteins at the level of sequence, it is likely to be highly homologous at the level of tertiary structure and may mediate protein dimerization. Hammond et al. (1998) considered SKI, SNO, and the dac-related proteins to be members of a gene super family. In situ hybridization revealed that mouse Dach mRNA is expressed in eye, limb, rib primordia, central nervous system, and genital eminence in embryos. Pax6 (OMIM Ref. No. 607108), the mammalian homolog of ey, and Dach show overlapping but nonidentical expression patterns. However, Dach expression in forebrain is unaffected in Pax6 mutant (small eye) mice, indicating that, at least in brain, Pax6 does not directly regulate Dach. The authors concluded that Dach joins a group of homologous eye genes shared by Drosophila and mouse, supporting the concept that a conserved genetic network operates in eye development in diverse organisms. In mice homozygously deleted for the Six6 (OMIM Ref. No. 606326) homeodomain factor, L1 et al. (2002) observed that Six6, in association with Dach corepressors, regulates early progenitor cell proliferation during mammalian retinogenesis and pituitary development by directly repressing cyclin-dependent kinase inhibitors, including the p27Kip1 (OMIM Ref. No. 600778) promoter. L1 et al. (2002) concluded that their data revealed a molecular mechanism by which a tissue-specific transcriptional repressor-corepressor complex can provide an organ-specific strategy for physiologic expansion of precursor populations. Animal model experiments lend further support to the function of DACH. Davis et al. (2001) generated Dach1-deficient mice which died during postnatal day 1 and exhibited failure to suckle, cyanosis, and respiratory distress. The authors hypothesized that a lack of morphologic defects in these mutant mice may be due to compensation by an additional Dach homolog.

It is appreciated that the abovementioned animal model for DACH is acknowledged by those skilled in the art as a scientifically valid animal model, as can be further appreciated from the publications sited hereinbelow.

Full details of the abovementioned studies are described in the following publications, the disclosure of which are hereby incorporated by reference:
Hammond, K. L.; Hanson, I. M.; Brown, A. G.; Lettice, L. A.; Hill, R. E.: Mammalian and Drosophila dachshund genes are related to the Ski proto-oncogene and are expressed in eye and limb. Mech. Dev. 74: 121-131, 1998.; and
Li, X.; Perissi, V.; Liu, F.; Rose, D. W.; Rosenfeld, M. G.: Tissue-specific regulation of retinal and pituitary precursor cell proliferation. Science 297: 1180-1183, 2002.

Further studies establishing the function and utilities of DACH are found in John Hopkins OMIM database record ID 603803, and in sited publications numbered 225-229 listed in the bibliography section hereinbelow, which are also hereby incorporated by reference. Extracellular Matrix Protein 2, Female Organ and Adipocyte Specific (ECM2, Accession NM_{—}001393) is another VGAM170 host target gene. ECM2 BINDING SITE is HOST TARGET binding site found in the 5′ untranslated region of mRNA encoded by ECM2, corresponding to a HOST TARGET binding site such as BINDING SITE I, BINDING SITE II or BINDING SITE III. Table 2 illustrates the complementarity of the nucleotide sequences of ECM2 BINDING SITE, designated SEQ ID:823, to the nucleotide sequence of VGAM170 RNA, herein designated VGAM RNA, also designated SEQ ID:505.

Another function of VGAM170 is therefore inhibition of Extracellular Matrix Protein 2, Female Organ and Adipocyte Specific (ECM2, Accession NM_{—}001393). Accordingly, utilities of VGAM170 include diagnosis, prevention and treatment of diseases and clinical conditions associated with ECM2. Endothelin Receptor Type A (EDNRA, Accession XM_{—}034331) is another VGAM170 host target gene. EDNRA BINDING SITE is HOST TARGET binding site found in the 3′ untranslated region of mRNA encoded by EDNRA, corresponding to a HOST TARGET binding site such as BINDING SITE I, BINDING SITE II or BINDING SITE III. Table 2 illustrates the complementarity of the nucleotide sequences of EDNRA BINDING SITE, designated SEQ ID:2681, to the nucleotide sequence of VGAM170 RNA, herein designated VGAM RNA, also designated SEQ ID:505.

Another function of VGAM170 is therefore inhibition of Endothelin Receptor Type A (EDNRA, Accession XM_{—}034331), a gene which binds endothelins, and induces intracellular calcium flux and arachidonic acid accumulation. Accordingly, utilities of VGAM170 include diagnosis, prevention and treatment of diseases and clinical conditions associated with EDNRA. The function of EDNRA has been established by previous studies. See 131244. The endothelin receptor with highest affinity for ET1 (OMIM Ref. No. 131240) has been called ETA. Cyr et al. (1991) isolated a cDNA clone of a human endothelin receptor from a placental cDNA library. The deduced amino acid sequence was 94% identical to the bovine endothelin ETA receptor and was judged to represent the human homolog. They assigned the ETRA gene to chromosome 4 by analysis of its segregation pattern in rodent/human hybrids. Hosoda et al. (1992) isolated and characterized the gene for the human endothelin-A receptor. Southern blot analyses demonstrated that it is present in single copy. The gene spans more than 40 kb and contains 8 exons and 7 introns. The transcription start site, determined by primer extension experiments, was 502 bp upstream of the methionine initiation codon. Using human/rodent somatic hybrid cell DNAs, Hosoda et al. (1992) also assigned the gene to chromosome 4. Northern blot analyses demonstrated a 4.3-kb mRNA in a wide variety of human tissues with the highest level in the aorta and a substantial level in cultured human mesangial cells. Endothelin-1 inhibits active Na—K transport by as much as 50% in the renal tubule and other tissues (Zeidel et al., 1989). Okafor and Delamere (2001) noted that the presence of low levels of ET1 in aqueous humor combined with the potential for release of ET1 from ciliary processes suggested that the crystalline lens could be exposed to ET1 in vivo. They studied the influence of ET1 on active Na—K transport in the porcine lens. Their results suggested that ET1 inhibited active lens Na—K transport by activating EDNRA and EDNRB. Activation of the ET receptors also caused an increase in cytoplasmic calcium concentration in cultured lens epithelial cells. Both responses to ET1 appear to have a tyrosine kinase step.

Full details of the abovementioned studies are described in the following publications, the disclosure of which are hereby incorporated by reference:
Hosoda, K.; Nakao, K.; Tamura, N.; Arai, H.; Ogawa, Y.; Suga, S.; Nakanishi, S.; Imura, H.: Organization, structure, chromosomal assignment, and expression of the gene encoding the human endothelin-A receptor. J. Biol. Chem. 267: 18797-18804, 1992.; and
Okafor, M. C.; Delamere, N. A.: The inhibitory influence of endothelin on active sodium-potassium transport in porcine lens. Invest. Ophthal. Vis. Sci. 42: 1018-1023, 2001.

Further studies establishing the function and utilities of EDNRA are found in John Hopkins OMIM database record ID 131243, and in sited publications numbered 2861-904, 514-51 and 905 listed in the bibliography section hereinbelow, which are also hereby incorporated by reference. Ectodermal-neural Cortex (with BTB-like domain) (ENC1, Accession NM_{—}003633) is another VGAM170 host target gene. ENC1 BINDING SITE is HOST TARGET binding site found in the 3′ untranslated region of mRNA encoded by ENC1, corresponding to a HOST TARGET binding site such as BINDING SITE I, BINDING SITE II or BINDING SITE III. Table 2 illustrates the complementarity of the nucleotide sequences of ENC1 BINDING SITE, designated SEQ ID: 1045, to the nucleotide sequence of VGAM170 RNA, herein designated VGAM RNA, also designated SEQ ID:505.

Another function of VGAM170 is therefore inhibition of Ectodermal-neural Cortex (with BTB-like domain) (ENC1, Accession NM_{—}003633), a gene which is an actin-binding protein involved in the regulation of neuronal process formation and in differentiation of neural crest cells. Accordingly, utilities of VGAM170 include diagnosis, prevention and treatment of diseases and clinical conditions associated with ENC1. The function of ENC1 has been established by previous studies. DNA damage and/or hyperproliferative signals activate wildtype p53 tumor suppressor protein (TP53; 191170), inducing cell cycle arrest or apoptosis. Mutations that inactivate p53 occur in 50% of all tumors. Polyak et al. (1997) used serial analysis of gene expression (SAGE) to evaluate cellular mRNA levels in a colorectal cancer cell line transfected with p53. Of 7,202 transcripts identified, only 14 were expressed at levels more than 10-fold higher in p53-expressing cells than in control cells. Polyak et al. (1997) termed these genes ‘p53-induced genes,’ or PIGs, several of which were predicted to encode redox-controlling proteins. They noted that reactive oxygen species (ROS) are potent inducers of apoptosis. Flow cytometric analysis showed that p53 expression induces ROS production, which increases as apoptosis progresses under some conditions. The authors stated that the PIG10 gene, also called ENC1, encodes an actin-binding protein. By screening fetal and adult hippocampus cDNA libraries using a brain development-related cDNA as the probe, Kim et al. (1998) obtained cDNAs encoding ENC1, which they called NRPB (nuclear-restricted protein/brain). Human and mouse ENC1 share 99% amino acid identity. The deduced 589-amino acid ENC1 protein has a 114-amino acid BTB/POZ-like domain in the alpha-helical N terminus and a beta sheet bearing a 50-amino acid ‘kelch’ motif repeated 6 times in the C terminus. The kelch motif invariably contains 2 adjacent glycine residues and shares homology with several actin-associated proteins, including the Drosophila kelch protein. Northern blot analysis detected abundant expression of a 5.5-kb ENC1 transcript in fetal brain, with moderate expression in fetal heart, lung, and kidney. In adult tissues, high levels of ENC1 were detected in brain, particularly in amygdala and hippocampus, and lower levels were detected in pancreas. In 12 day-but not 10 day-postcoitus mouse embryos, expression of Enc1 was 50-fold higher in brain than in other tissues. Immunoprecipitation and Western blot analysis showed that ENC1 is expressed as a 67-kD protein in nuclear pellets and as 67- and 57-kD proteins in total cell lysates from primary neurons. Western blot analysis, immunofluorescence, and confocal microscopy demonstrated that Enc1 is expressed in the nuclear matrix of rat hippocampal neurons but not at all in astrocytes. By searching an EST database for homologs of mouse Enc1, Hernandez et al. (1998) identified human ENC1. Northern blot analysis detected abundant expression of an approximately 4.5-kb ENC1 transcript in brain, with lower expression in pancreas and no expression in other tissues. Within the central nervous system, expression was highest in cerebral cortex, frontal and temporal lobes, putamen, and spinal cord; lower expression was found in medulla and cerebellum, and very low levels of expression were found in the occipital pole. Low levels of ENC1 were also detected in a variety of neural tumor cell lines. ENC1 expression increased dramatically in a neuroblastoma cell line undergoing retinoic acid-induced differentiation. By differential display, Zhao et al. (2000) identified rat Enc1 as a transcript associated with differentiation of rat preadipocytes in primary culture. Using the fragment identified by differential display as probe, they cloned full-length Enc1 cDNA from a mouse brain cDNA library. By Northern blot analysis of rat tissues, Zhao et al. (2000) found high expression in brain, low expression in testis, and no expression in other tissues tested. They also found high expression of Enc1 in the stroma-vascular fraction of adipose tissue but very little in mature adipocyte fraction. Transient transfection in a 3T3 fibroblastic preadipocyte cell line resulted in subcellular colocalization with F-actin (OMIM Ref. No. 102560). Kim et al. (1998) showed that expression of ENC1 induced neuronal process formation, whereas antisense treatment inhibited neurite development. Immunoblot analysis showed that ENC1 can be phosphorylated and binds to the functionally active hypophosphorylated form of the nuclear matrix protein RB1 (OMIM Ref. No. 180200) during neuronal differentiation. Using primary cell culture of rat stroma-vascular cells, Zhao et al. (2000) found that transient early expression of Enc1 preceded the conversion of the fibroblastic preadipocytes to mature adipose. Enc1 expression also preceded expression of adipocyte-specific markers, including transcription factors known to activate adipocyte genes. Antisense transfection blocked differentiation to the mature adipocyte morphology.

Full details of the abovementioned studies are described in the following publications, the disclosure of which are hereby incorporated by reference:
Zhao, L.; Gregoire, F.; Sul, H. S.: Transient induction of ENC-1, a kelch-related actin-binding protein, is required for adipocyte differentiation. J. Biol. Chem. 275: 16845-16850, 2000.; and
Hernandez, M.-C.; Andres-Barquin, P. J.; Israel, M. A.: Assignment of the ectodermal-neural cortex 1 gene (Enc1) to mouse chromosome band 13D1 by fluorescence in situ hybridization. Cy.

Further studies establishing the function and utilities of ENC1 are found in John Hopkins OMIM database record ID 605173, and in sited publications numbered 538-541, 53 and 1073 listed in the bibliography section hereinbelow, which are also hereby incorporated by reference. Fanconi Anemia, Complementation Group F (FANCF, Accession NM_{—}022725) is another VGAM170 host target gene. FANCF BINDING SITE is HOST TARGET binding site found in the 3′ untranslated region of mRNA encoded by FANCF, corresponding to a HOST TARGET binding site such as BINDING SITE I, BINDING SITE II or BINDING SITE III. Table 2 illustrates the complementarity of the nucleotide sequences of FANCF BINDING SITE, designated SEQ ID:1999, to the nucleotide sequence of VGAM170 RNA, herein designated VGAM RNA, also designated SEQ ID:505.

Another function of VGAM170 is therefore inhibition of Fanconi Anemia, Complementation Group F (FANCF, Accession NM_{—}022725). Accordingly, utilities of VGAM170 include diagnosis, prevention and treatment of diseases and clinical conditions associated with FANCF. Fc Fragment of IgA, Receptor For (FCAR, Accession NM_{—}133279) is another VGAM170 host target gene. FCAR BINDING SITE1 and FCAR BINDING SITE2 are HOST TARGET binding sites found in untranslated regions of mRNA encoded by FCAR, corresponding to HOST TARGET binding sites such as BINDING SITE I, BINDING SITE II or BINDING SITE III. Table 2 illustrates the complementarity of the nucleotide sequences of FCAR BINDING SITE1 and FCAR BINDING SITE2, designated SEQ ID:2405 and SEQ ID:2407 respectively, to the nucleotide sequence of VGAM170 RNA, herein designated VGAM RNA, also designated SEQ ID:505.

Another function of VGAM170 is therefore inhibition of Fc Fragment of IgA, Receptor For (FCAR, Accession NM_{—}133279), a gene which binds to the fc region of immunoglobulins alpha and mediates several functions including cytokine production. Accordingly, utilities of VGAM170 include diagnosis, prevention and treatment of diseases and clinical conditions associated with FCAR. The function of FCAR has been established by previous studies. Human Fc-alpha receptor (FCAR) is present on a number of cell types, including neutrophils, monocytes, macrophages, and eosinophils. FCAR interacts with aggregated IgAs, such as IgA coated on the surface of an invading microorganism, and mediates several immunologic defense processes such as phagocytosis, antibody-dependent cell-mediated cytotoxicity, and stimulation of the release of inflammatory mediators. FCAR is a glycoprotein of 50 to 100 kD, with diversity on different cell types. Narita et al. (2001) examined polymorphisms in the promoter and 5-prime untranslated region of the FCAR gene in 151 patients with IgA nephropathy and 163 patients with other glomerular diseases shown to have no mesangial IgA deposition by renal biopsy. Haplotype analysis showed tight linkage disequilibrium among the polymorphisms. No significant association for the genotype, allele, and haplotype frequencies of the polymorphisms were shown between the patients with histologically proven IgA nephropathy and those with other glomerular diseases. Thus, the analyzed polymorphisms did not appear to be primarily involved in susceptibility to IgA nephropathy.

Full details of the abovementioned studies are described in the following publications, the disclosure of which are hereby incorporated by reference:
Narita, I.; Goto, S.; Saito, N.; Sakatsume, M.; Jin, S.; Omori, K.; Gejyo, F.: Genetic polymorphisms in the promoter and 5-prime UTR region of the Fc alpha receptor (CD89) are not associated with a risk of IgA nephropathy. J. Hum. Genet. 46: 694-698, 2001.; and
Maliszewski, C. R.; March, C. J.; Schoenborn, M. A.; Gimpel, S.; Shen, L.: Expression cloning of a human Fc receptor for IgA. J. Exp. Med. 172: 1665-1672, 1990.

Further studies establishing the function and utilities of FCAR are found in John Hopkins OMIM database record ID 147045, and in sited publications numbered 712-714, 709-71 and 715-716 listed in the bibliography section hereinbelow, which are also hereby incorporated by reference. Potassium Inwardly-rectifying Channel, Sub family J, Member 5 (KCNJ5, Accession NM_{—}000890) is another VGAM170 host target gene. KCNJ5 BINDING SITE is HOST TARGET binding site found in the 5′ untranslated region of mRNA encoded by KCNJ5, corresponding to a HOST TARGET binding site such as BINDING SITE I, BINDING SITE II or BINDING SITE III. Table 2 illustrates the complementarity of the nucleotide sequences of KCNJ5 BINDING SITE, designated SEQ ID:784, to the nucleotide sequence of VGAM170 RNA, herein designated VGAM RNA, also designated SEQ ID:505.

Another function of VGAM170 is therefore inhibition of Potassium Inwardly-rectifying Channel, Sub family J, Member 5 (KCNJ5, Accession NM_{—}000890), a gene which is a potassium inwardly-rectifying channel. Accordingly, utilities of VGAM170 include diagnosis, prevention and treatment of diseases and clinical conditions associated with KCNJ5. The function of KCNJ5 has been established by previous studies. Potassium channels inhibited by cytosolic ATP are found in a wide variety of tissues. Tucker et al. (1995) noted that in the pancreatic beta-cell, potassium channels play a critical role in the regulation of insulin secretion, and in smooth muscle they are responsible for hypoxic vasodilatation. Moreover, these channels are the targets for several important classes of therapeutic drugs, including the antidiabetic sulfonamides and the antihypertensive potassium channel openers. In the heart, as in other tissues, K(ATP) channels are thought to couple the membrane potential to the metabolic status of the cell, and these normally quiescent channels are activated during transient ischemic and hypoxic periods when they contribute to shortening of the cardiac action potential duration. Ashford et al. (1994) cloned the rat heart K(ATP) channel, thus enabling the isolation of the human homolog. The primary structure of KATP1 placed it in the J sub family of inwardly rectifying potassium channels (Bond et al., 1994), such as KCNJ2 (OMIM Ref. No. 600681) and KCNJ4 (OMIM Ref. No. 600504); thus, the human homolog was designated KCNJ5. Wickman et al. (1997) reported a partial sequence of human GIRK4. They used human/rodent somatic cell hybrids to localize the human gene to chromosome 11, consistent with previous studies that localized the gene to 11q23-ter. Wickman et al. (1997) cloned the mouse Girk4 gene. They showed that the gene is expressed almost exclusively in the mouse heart. Using interspecific backcross analysis, Wickman et al. (1997) mapped the mouse Girk4 gene to chromosome 9, consistent with the mapping to human chromosome 11.

Full details of the abovementioned studies are described in the following publications, the disclosure of which are hereby incorporated by reference:
Ashford, M. L. J.; Bond, C. T.; Blair, T. A.; Adelman, J. P.: Cloning and functional expression of a rat heart KATP channel. Nature 370: 456-459, 1994.; and
Bond, C. T.; Pessia, M.; Xia, X.-M.; Lagrutta, A.; Kavanaugh, M. P.; Adelman, J. P.: Cloning and expression of a family of inward rectifier potassium channels. Receptors Channels 2: 183.

Further studies establishing the function and utilities of KCNJ5 are found in John Hopkins OMIM database record ID 600734, and in sited publications numbered 1692-1695 listed in the bibliography section hereinbelow, which are also hereby incorporated by reference. MADS Box Transcription Enhancer Factor 2, Polypeptide A (myocyte enhancer factor 2A) (MEF2A, Accession NM_{—}005587) is another VGAM170 host target gene. MEF2A BINDING SITE is HOST TARGET binding site found in the 3′ untranslated region of mRNA encoded by MEF2A, corresponding to a HOST TARGET binding site such as BINDING SITE I, BINDING SITE II or BINDING SITE III. Table 2 illustrates the complementarity of the nucleotide sequences of MEF2A BINDING SITE, designated SEQ ID:1228, to the nucleotide sequence of VGAM170 RNA, herein designated VGAM RNA, also designated SEQ ID:505.

Another function of VGAM170 is therefore inhibition of MADS Box Transcription Enhancer Factor 2, Polypeptide A (myocyte enhancer factor 2A) (MEF2A, Accession NM_{—}005587), a gene which binds a consensus sequence that regulates transcription. Accordingly, utilities of VGAM170 include diagnosis, prevention and treatment of diseases and clinical conditions associated with MEF2A. The function of MEF2A and its association with various diseases and clinical conditions, has been established by previous studies, as described hereinabove with reference to VGAM73. MIR16 (Accession NM_{—}016641) is another VGAM170 host target gene. MIR16 BINDING SITE is HOST TARGET binding site found in the 3′ untranslated region of mRNA encoded by MIR16, corresponding to a HOST TARGET binding site such as BINDING SITE I, BINDING SITE II or BINDING SITE III. Table 2 illustrates the complementarity of the nucleotide sequences of MIR16 BINDING SITE, designated SEQ ID:1704, to the nucleotide sequence of VGAM170 RNA, herein designated VGAM RNA, also designated SEQ ID:505.

Another function of VGAM170 is therefore inhibition of MIR16 (Accession NM_{—}016641), a gene which is a membrane interacting protein. Accordingly, utilities of VGAM170 include diagnosis, prevention and treatment of diseases and clinical conditions associated with MIR16. The function of MIR16 has been established by previous studies. Using a yeast 2-hybrid screen to identify proteins that interact with RGS16 (OMIM Ref. No. 602514), Zheng et al. (2000) isolated a cDNA encoding rat Mir16. By searching sequence databases with rat Mir16 as the probe, they identified a cDNA encoding human MIR16 that had been reported by Loftus et al. (1999) as part of a large-scale chromosome 16 sequencing effort. The human MIR16 protein shares 94% amino acid similarity with rat MIR16 and also shares strong homology with bacterial glycerophosphodiester phosphodiesterases. Northern blot analysis detected widespread expression of a 1.8-kb Mir16 transcript in rat tissues, with highest levels in heart, brain, liver, kidney, and testis. Similar expression was observed for human and mouse MIR16. Using yeast 2-hybrid and GST pull-down assays, Zheng et al. (2000) demonstrated that MIR16 interacts with the RGS domain of RGS16 and weakly with other RGS proteins, including RGS2 (OMIM Ref. No. 600861). Membrane association assays and endoglycosidase H digestion showed that MIR16 is an integral membrane glycoprotein. Immunofluorescence and immunoelectron microscopy localized Mir16 in the intracellular membranes in rat pituitary and on the plasma membrane in rat liver and kidney. By genomic sequence analysis, Loftus et al. (1999) mapped the MIR16 gene to chromosome 16p.

Full details of the abovementioned studies are described in the following publications, the disclosure of which are hereby incorporated by reference:
Loftus, B. J.; Kim, U.-J.; Sneddon, V. P.; Kalush, F.; Brandon, R.; Fuhrmann, J.; Mason, T.; Crosby, M. L.; Barnstead, M.; Cronin, L.; Mays, A. D.; Cao, Y.; Xu, R. X.; Kang, H.-L.; Mitchell, S.; Eichler, E. E.; Harris, P. C.; Venter, J. C.; Adams, M. D.: Genome duplications and other features in 12 Mb of DNA sequence from human chromosome 16p and 16q. Genomics 60: 295-308, 1999.; and
Zheng, B.; Chen, D.; Farquhar, M. G.: MIR16, a putative membrane glycerophosphodiester phosphodiesterase, interacts with RGS16. Proc. Nat. Acad. Sci. 97: 3999-4004, 2000.

Further studies establishing the function and utilities of MIR16 are found in John Hopkins OMIM database record ID 605943, and in sited publications numbered 153 and 1928 listed in the bibliography section hereinbelow, which are also hereby incorporated by reference. Procollagen-proline, 2-oxoglutarate 4-dioxygenase (proline 4-hydroxylase), Beta Polypeptide (protein disulfide isomerase; thyroid hormone binding protein p55) (P4HB, Accession NM_{—}000918) is another VGAM170 host target gene. P4HB BINDING SITE is HOST TARGET binding site found in the 3′ untranslated region of mRNA encoded by P4HB, corresponding to a HOST TARGET binding site such as BINDING SITE I, BINDING SITE II or BINDING SITE III. Table 2 illustrates the complementarity of the nucleotide sequences of P4HB BINDING SITE, designated SEQ ID:787, to the nucleotide sequence of VGAM170 RNA, herein designated VGAM RNA, also designated SEQ ID:505.

Another function of VGAM170 is therefore inhibition of Procollagen-proline, 2-oxoglutarate 4-dioxygenase (proline 4-hydroxylase), Beta Polypeptide (protein disulfide isomerase; thyroid hormone binding protein p55) (P4HB, Accession NM_{—}000918), a gene which catalyzes formation of 4-hydroxyproline in collagens. Accordingly, utilities of VGAM170 include diagnosis, prevention and treatment of diseases and clinical conditions associated with P4HB. The function of P4HB has been established by previous studies. Prolyl 4-hydroxylase (EC 1.14.11.2) is involved in hydroxylation of prolyl residues in preprocollagen. Pihlajaniemi et al. (1987) cloned the PROHB gene. Prolyl 4-hydroxylase is a tetramer consisting of 2 alpha (176710, 600608) and 2 beta subunits of molecular weights about 64,000 and 60,000, respectively, for the monomers. Characterization of cDNA clones for the human beta subunit indicated that the polypeptide is 508 amino acids long, including a signal peptide of 17 amino acids. Pihlajaniemi et al. (1987) also found that disulfide isomerase (EC 5.3.4.1) is a product of the same gene. When present in cells in monomeric form, the protein serves the function of DSI (Koivu et al., 1987); when present in the prolyl 4-hydroxylase tetramer, it catalyzes the formation of 4-hydroxyproline in collagen. Cheng et al. (1987) demonstrated by molecular cloning and nucleotide sequencing that cellular thyroid hormone-binding protein is also identical to the beta subunit of prolyl 4-hydroxylase and protein disulfide isomerase. Tasanen et al. (1988) isolated genomic clones for the human gene coding for this multifunctional protein. They found that the gene is about 18 kb long and consists of 11 exons. The codons for the 2 presumed active sites of protein disulfide isomerase, each a cys-gly-his-cys sequence, were found to be located 12 bp from the beginning of exons 2 and 9. Another of the many functions of protein disulfide isomerase is its role as the smaller element of the heterodimeric microsomal triglyceride transfer protein (MTP; 157147). The unique larger subunit of this heterodimer is mutant in patients with abetalipoproteinemia (OMIM Ref. No. 200100). Since chylomicrons, very low density lipoproteins, and low density lipoproteins are absent from the plasma in abetalipoproteinemic subjects, and since the clinical pathology of abetalipoproteinemia results from deficiency of fat-soluble vitamins that are transported on apoB-containing lipoproteins, Sharp et al. (1993) proposed that inhibition of MTP may provide a specific mechanism for lowering plasma cholesterol and triglyceride levels.

Full details of the abovementioned studies are described in the following publications, the disclosure of which are hereby incorporated by reference:
Pihlajaniemi, T.; Helaakoski, T.; Tasanen, K.; Myllyla, R.; Huhtala, M.-L.; Koivu, J.; Kivirikko, K. I.: Molecular cloning of the beta-subunit of human prolyl 4-hydroxylase: this subunit and protein disulphide isomerase are products of the same gene. EMBO J. 6: 643-649, 1987.; and
Sharp, D.; Blinderman, L.; Combs, K. A.; Kienzle, B.; Ricci, B.; Wager-Smith, K.; Gil, C. M.; Turck, C. W.; Bouma, M.-E.; Rader, D. J.; Aggerbeck, L. P.; Gregg, R. E.; Gordon, D. A.; We.

Further studies establishing the function and utilities of P4HB are found in John Hopkins OMIM database record ID 176790, and in sited publications numbered 2171-2180, 75 and 2181 listed in the bibliography section hereinbelow, which are also hereby incorporated by reference. V-raf-1 Murine Leukemia Viral Oncogene Homolog 1 (RAF1, Accession XM_{—}087425) is another VGAM170 host target gene. RAF1 BINDING SITE is HOST TARGET binding site found in the 3′ untranslated region of mRNA encoded by RAF1, corresponding to a HOST TARGET binding site such as BINDING SITE I, BINDING SITE II or BINDING SITE III. Table 2 illustrates the complementarity of the nucleotide sequences of RAF1 BINDING SITE, designated SEQ ID:3168, to the nucleotide sequence of VGAM170 RNA, herein designated VGAM RNA, also designated SEQ ID:505.

Another function of VGAM170 is therefore inhibition of V-raf-1 Murine Leukemia Viral Oncogene Homolog 1 (RAF1, Accession XM_{—}087425). Accordingly, utilities of VGAM170 include diagnosis, prevention and treatment of diseases and clinical conditions associated with RAF1. Ribosomal Protein S6 Kinase, 90 kDa, Polypeptide 5 (RPS6KA5, Accession NM_{—}004755) is another VGAM170 host target gene. RPS6KA5 BINDING SITE is HOST TARGET binding site found in the 3′ untranslated region of mRNA encoded by RPS6KA5, corresponding to a HOST TARGET binding site such as BINDING SITE I, BINDING SITE II or BINDING SITE III. Table 2 illustrates the complementarity of the nucleotide sequences of RPS6KA5 BINDING SITE, designated SEQ ID:1153, to the nucleotide sequence of VGAM170 RNA, herein designated VGAM RNA, also designated SEQ ID:505.

Another function of VGAM170 is therefore inhibition of Ribosomal Protein S6 Kinase, 90 kDa, Polypeptide 5 (RPS6KA5, Accession NM_{—}004755), a gene which plays an essential role in the proliferation of yeast cells. Accordingly, utilities of VGAM170 include diagnosis, prevention and treatment of diseases and clinical conditions associated with RPS6KA5. The function of RPS6KA5 has been established by previous studies. Members of the extracellular signal-regulated kinase (ERK) sub family of the mitogen-activated protein kinases (MAPKs) are activated by growth factors (see OMIM Ref. No. ERK2, 176948), while stress-activated protein kinase (SAPK) sub family members are strongly activated by stress signals (see OMIM Ref. No. SAPK4, 602899). MAPKAP-K1 (see OMIM Ref. No. RPS6KA1, 601684) isoforms appear to be in vivo substrates for ERKs, while MAPKAP-K2 (OMIM Ref. No. 602006) and MAPKAP-K3 (OMIM Ref. No. 602130) are in vivo substrates for SAPK2A (OMIM Ref. No. 600289) and SAPK2B (OMIM Ref. No. 602898). The MAPKAP-K1 proteins each contain 2 protein kinase domains within a single polypeptide, and 1 role of the C-terminal kinase domain is to activate the N-terminal kinase domain. By searching an EST database with the sequence of the MAPKAP-K1 N-terminal kinase domain, Deak et al. (1998) identified cDNAs encoding 2 novel kinases: mitogen- and stress-activated protein kinase-1 (MSK1) and mitogen- and stress-activated protein kinase-2 (OMIM Ref. No. 603606). The predicted 802-amino acid MSK1 protein contains 2 protein kinase domains, each of which includes the 11 subdomains characteristic of all protein kinases. MSK1 shares 43% protein sequence identity with the MAPKAP-K1 isoforms. Northern blot analysis indicated that MSK1 was expressed as a 4-kb mRNA in all tissues tested, with the highest levels of expression in brain, muscle, and placenta. Immunoelectron microscopy localized MSK1 to the nucleus. MSK1 was activated in vitro and in vivo by either ERK or SAPK2 proteins. Deak et al. (1998) presented evidence suggesting that MSK1, rather than MAPKAP-K1 or MAPKAP-K2/K3, mediates activation of the cAMP response element-binding protein (see OMIM Ref. No. CREB1, 123810) and activating transcription factor-1 (OMIM Ref. No. 123803) by either growth factors or stress signals. By radiation hybrid analysis, Jiang et al. (1999) mapped the RPS6KA5 gene to chromosome 14q31-q32.

Full details of the abovementioned studies are described in the following publications, the disclosure of which are hereby incorporated by reference:
Deak, M.; Clifton, A. D.; Lucocq, J. M.; Alessi, D. R.: Mitogen- and stress-activated protein kinase-1 (MSK1) is directly activated by MAPK and SAPK2/p38, and may mediate activation of CREB. EMBO J. 17: 4426-4441, 1998.; and
Jiang, C.; Yu, L.; Tu, Q.; Zhao, Y.; Zhang, H.; Zhao, S.: Assignment of a member of the ribosomal protein S6 kinase family, RPS6KA5, to human chromosome 14q31-q32.1 by radiation hybrid map.

Further studies establishing the function and utilities of RPS6KA5 are found in John Hopkins OMIM database record ID 603607, and in sited publications numbered 665-666 listed in the bibliography section hereinbelow, which are also hereby incorporated by reference. Transducin (beta)-like 1X-linked (TBL1X, Accession NM_{—}005647) is another VGAM170 host target gene. TBL1X BINDING SITE is HOST TARGET binding site found in the 3′ untranslated region of mRNA encoded by TBL1X, corresponding to a HOST TARGET binding site such as BINDING SITE I, BINDING SITE II or BINDING SITE III. Table 2 illustrates the complementarity of the nucleotide sequences of TBL1X BINDING SITE, designated SEQ ID:1233, to the nucleotide sequence of VGAM170 RNA, herein designated VGAM RNA, also designated SEQ ID:505.

Another function of VGAM170 is therefore inhibition of Transducin (beta)-like 1X-linked (TBL1X, Accession NM_{—}005647), a gene which activates latent HDAC3 activity. Accordingly, utilities of VGAM170 include diagnosis, prevention and treatment of diseases and clinical conditions associated with TBL1X. The function of TBL1X has been established by previous studies. In the course of constructing a deletion map of the distal portion of the short arm of the X chromosome and the identification of the OA1 gene (OMIM Ref. No. 300500), Bassi et al. (1999) performed cDNA selection experiments that resulted in the isolation of a novel gene, TBL1, located outside the OA1 critical region on the telomeric side. The TBL1 gene maps to the Xp22.3 region and shares significant homology with members of the WD40 repeat-containing protein family. The open reading frame encodes a 526-amino acid protein containing 6 beta-transducin repeats (WD40 motif) in the C-terminal domain. The homology with known beta-subunits of G proteins and other WD40 repeat-containing proteins is restricted to the WD40 motif. Northern blot analysis indicated that the TBL1 gene is ubiquitously expressed as 2 transcripts of approximately 2.1 and 6.0 kb. Matsuzawa and Reed (2001) elucidated a network of protein interactions in which SIAH1 (OMIM Ref. No. 602212), SIP (OMIM Ref. No. 606186), SKP1 (OMIM Ref. No. 601434), and EBI collaborate in a pathway controlling beta-catenin (OMIM Ref. No. 116806) levels, affecting activity of beta-catenin-dependent TCF (e.g., TCF1; 142410) and LEF (e.g., LEF1; 153245) transcription factors. This pathway is inducible by p53 (OMIM Ref. No. 191170), revealing a link between genotoxic injury responses and beta-catenin degradation. SIAH1 is physically linked to EBI by association with SIP, which binds SKP1.

Full details of the abovementioned studies are described in the following publications, the disclosure of which are hereby incorporated by reference:
Bassi, M. T.; Ramesar, R. S.; Caciotti, B.; Winship, I. M.; De Grandi, A.; Riboni, M.; Townes, P. L.; Beighton, P.; Ballabio, A.; Borsani, G.: X-linked late-onset sensorineural deafness caused by a deletion involving OA1 and a novel gene containing WD-40 repeats. Am. J. Hum. Genet. 64: 1604-1616, 1999.; and
Matsuzawa, S.; Reed, J. C.: Siah-1, SIP, and Ebi collaborate in a novel pathway for beta-catenin degradation linked to p53 responses. Molec. Cell 7: 915-926, 2001.

Further studies establishing the function and utilities of TBL1X are found in John Hopkins OMIM database record ID 300196, and in sited publications numbered 2640-2643, 164 and 2644-2645 listed in the bibliography section hereinbelow, which are also hereby incorporated by reference. ARHGAP10 (Accession NM_{—}020824) is another VGAM170 host target gene. ARHGAP10 BINDING SITE is HOST TARGET binding site found in the 5′ untranslated region of mRNA encoded by ARHGAP10, corresponding to a HOST TARGET binding site such as BINDING SITE I, BINDING SITE II or BINDING SITE III. Table 2 illustrates the complementarity of the nucleotide sequences of ARHGAP10 BINDING SITE, designated SEQ ID:1926, to the nucleotide sequence of VGAM170 RNA, herein designated VGAM RNA, also designated SEQ ID:505.

Another function of VGAM170 is therefore inhibition of ARHGAP10 (Accession NM_{—}020824). Accordingly, utilities of VGAM170 include diagnosis, prevention and treatment of diseases and clinical conditions associated with ARHGAP10. ATPase, Class II, Type 9A (ATP9A, Accession XM_{—}030577) is another VGAM170 host target gene. ATP9A BINDING SITE is HOST TARGET binding site found in the 3′ untranslated region of mRNA encoded by ATP9A, corresponding to a HOST TARGET binding site such as BINDING SITE I, BINDING SITE II or BINDING SITE III. Table 2 illustrates the complementarity of the nucleotide sequences of ATP9A BINDING SITE, designated SEQ ID:2619, to the nucleotide sequence of VGAM170 RNA, herein designated VGAM RNA, also designated SEQ ID:505.

Another function of VGAM170 is therefore inhibition of ATPase, Class II, Type 9A (ATP9A, Accession XM_{—}030577). Accordingly, utilities of VGAM170 include diagnosis, prevention and treatment of diseases and clinical conditions associated with ATP9A. Di-Ras2 (Accession NM_{—}017594) is another VGAM170 host target gene. Di-Ras2 BINDING SITE is HOST TARGET binding site found in the 3′ untranslated region of mRNA encoded by Di-Ras2, corresponding to a HOST TARGET binding site such as BINDING SITE I, BINDING SITE II or BINDING SITE III. Table 2 illustrates the complementarity of the nucleotide sequences of Di-Ras2 BINDING SITE, designated SEQ ID:1725, to the nucleotide sequence of VGAM170 RNA, herein designated VGAM RNA, also designated SEQ ID:505.

Another function of VGAM170 is therefore inhibition of Di-Ras2 (Accession NM_{—}017594). Accordingly, utilities of VGAM170 include diagnosis, prevention and treatment of diseases and clinical conditions associated with Di-Ras2. Formin Homology 2 Domain Containing 2 (FHOD2, Accession XM_{—}057927) is another VGAM170 host target gene. FHOD2 BINDING SITE is HOST TARGET binding site found in the 3′ untranslated region of mRNA encoded by FHOD2, corresponding to a HOST TARGET binding site such as BINDING SITE I, BINDING SITE II or BINDING SITE III. Table 2 illustrates the complementarity of the nucleotide sequences of FHOD2 BINDING SITE, designated SEQ ID:2981, to the nucleotide sequence of VGAM170 RNA, herein designated VGAM RNA, also designated SEQ ID:505.

Another function of VGAM170 is therefore inhibition of Formin Homology 2 Domain Containing 2 (FHOD2, Accession XM_{—}057927). Accordingly, utilities of VGAM170 include diagnosis, prevention and treatment of diseases and clinical conditions associated with FHOD2. FLJ10704 (Accession NM_{—}018185) is another VGAM170 host target gene. FLJ10704 BINDING SITE is HOST TARGET binding site found in the 3′ untranslated region of mRNA encoded by FLJ10704, corresponding to a HOST TARGET binding site such as BINDING SITE I, BINDING SITE II or BINDING SITE III. Table 2 illustrates the complementarity of the nucleotide sequences of FLJ10704 BINDING SITE, designated SEQ ID: 1795, to the nucleotide sequence of VGAM170 RNA, herein designated VGAM RNA, also designated SEQ ID:505.

Another function of VGAM170 is therefore inhibition of FLJ10704 (Accession NM_{—}018185). Accordingly, utilities of VGAM170 include diagnosis, prevention and treatment of diseases and clinical conditions associated with FLJ10704. FLJ12085 (Accession NM_{—}022771) is another VGAM170 host target gene. FLJ12085 BINDING SITE is HOST TARGET binding site found in the 3′ untranslated region of mRNA encoded by FLJ12085, corresponding to a HOST TARGET binding site such as BINDING SITE I, BINDING SITE II or BINDING SITE III. Table 2 illustrates the complementarity of the nucleotide sequences of FLJ12085 BINDING SITE, designated SEQ ID:2006, to the nucleotide sequence of VGAM170 RNA, herein designated VGAM RNA, also designated SEQ ID:505.

Another function of VGAM170 is therefore inhibition of FLJ12085 (Accession NM_{—}022771). Accordingly, utilities of VGAM170 include diagnosis, prevention and treatment of diseases and clinical conditions associated with FLJ12085. FLJ20209 (Accession XM_{—}098142) is another VGAM170 host target gene. FLJ20209 BINDING SITE is HOST TARGET binding site found in the 3′ untranslated region of mRNA encoded by FLJ20209, corresponding to a HOST TARGET binding site such as BINDING SITE I, BINDING SITE II or BINDING SITE III. Table 2 illustrates the complementarity of the nucleotide sequences of FLJ20209 BINDING SITE, designated SEQ ID:3351, to the nucleotide sequence of VGAM170 RNA, herein designated VGAM RNA, also designated SEQ ID:505.

Another function of VGAM170 is therefore inhibition of FLJ20209 (Accession XM_{—}098142). Accordingly, utilities of VGAM170 include diagnosis, prevention and treatment of diseases and clinical conditions associated with FLJ20209. FLJ22174 (Accession NM_{—}021945) is another VGAM170 host target gene. FLJ22174 BINDING SITE is HOST TARGET binding site found in the 5′ untranslated region of mRNA encoded by FLJ22174, corresponding to a HOST TARGET binding site such as BINDING SITE I, BINDING SITE II or BINDING SITE III. Table 2 illustrates the complementarity of the nucleotide sequences of FLJ22174 BINDING SITE, designated SEQ ID:1969, to the nucleotide sequence of VGAM170 RNA, herein designated VGAM RNA, also designated SEQ ID:505.

Another function of VGAM170 is therefore inhibition of FLJ22174 (Accession NM_{—}021945). Accordingly, utilities of VGAM170 include diagnosis, prevention and treatment of diseases and clinical conditions associated with FLJ22174. HSPC129 (Accession NM_{—}016396) is another VGAM170 host target gene. HSPC129 BINDING SITE is HOST TARGET binding site found in the 3′ untranslated region of mRNA encoded by HSPC129, corresponding to a HOST TARGET binding site such as BINDING SITE I, BINDING SITE II or BINDING SITE III. Table 2 illustrates the complementarity of the nucleotide sequences of HSPC129 BINDING SITE, designated SEQ ID: 1685, to the nucleotide sequence of VGAM170 RNA, herein designated VGAM RNA, also designated SEQ ID:505.

Another function of VGAM170 is therefore inhibition of HSPC129 (Accession NM_{—}016396). Accordingly, utilities of VGAM170 include diagnosis, prevention and treatment of diseases and clinical conditions associated with HSPC129. KIAA0632 (Accession NM_{—}015545) is another VGAM170 host target gene. KIAA0632 BINDING SITE is HOST TARGET binding site found in the 3′ untranslated region of mRNA encoded by KIAA0632, corresponding to a HOST TARGET binding site such as BINDING SITE I, BINDING SITE II or BINDING SITE III. Table 2 illustrates the complementarity of the nucleotide sequences of KIAA0632 BINDING SITE, designated SEQ ID: 1634, to the nucleotide sequence of VGAM170 RNA, herein designated VGAM RNA, also designated SEQ ID:505.

Another function of VGAM170 is therefore inhibition of KIAA0632 (Accession NM_{—}015545). Accordingly, utilities of VGAM170 include diagnosis, prevention and treatment of diseases and clinical conditions associated with KIAA0632. KIAA1184 (Accession NM_{—}022572) is another VGAM170 host target gene. KIAA1184 BINDING SITE is HOST TARGET binding site found in the 3′ untranslated region of mRNA encoded by KIAA1184, corresponding to a HOST TARGET binding site such as BINDING SITE I, BINDING SITE II or BINDING SITE III. Table 2 illustrates the complementarity of the nucleotide sequences of KIAA1184 BINDING SITE, designated SEQ ID:1994, to the nucleotide sequence of VGAM170 RNA, herein designated VGAM RNA, also designated SEQ ID:505.

Another function of VGAM170 is therefore inhibition of KIAA1184 (Accession NM_{—}022572). Accordingly, utilities of VGAM170 include diagnosis, prevention and treatment of diseases and clinical conditions associated with KIAA1184. KIAA1671 (Accession XM_{—}037809) is another VGAM170 host target gene. KIAA1671 BINDING SITE is HOST TARGET binding site found in the 3′ untranslated region of mRNA encoded by KIAA1671, corresponding to a HOST TARGET binding site such as BINDING SITE I, BINDING SITE II or BINDING SITE III. Table 2 illustrates the complementarity of the nucleotide sequences of KIAA1671 BINDING SITE, designated SEQ ID:2725, to the nucleotide sequence of VGAM170 RNA, herein designated VGAM RNA, also designated SEQ ID:505.

Another function of VGAM170 is therefore inhibition of KIAA1671 (Accession XM_{—}037809). Accordingly, utilities of VGAM170 include diagnosis, prevention and treatment of diseases and clinical conditions associated with KIAA1671. KIAA1728 (Accession XM_{—}043492) is another VGAM170 host target gene. KIAA1728 BINDING SITE is HOST TARGET binding site found in the 3′ untranslated region of mRNA encoded by KIAA1728, corresponding to a HOST TARGET binding site such as BINDING SITE I, BINDING SITE II or BINDING SITE III. Table 2 illustrates the complementarity of the nucleotide sequences of KIAA1728 BINDING SITE, designated SEQ ID:2818, to the nucleotide sequence of VGAM170 RNA, herein designated VGAM RNA, also designated SEQ ID:505.

Another function of VGAM170 is therefore inhibition of KIAA1728 (Accession XM_{—}043492). Accordingly, utilities of VGAM170 include diagnosis, prevention and treatment of diseases and clinical conditions associated with KIAA1728. KIAA1737 (Accession XM_{—}041115) is another VGAM170 host target gene. KIAA1737 BINDING SITE is HOST TARGET binding site found in the 3′ untranslated region of mRNA encoded by KIAA1737, corresponding to a HOST TARGET binding site such as BINDING SITE I, BINDING SITE II or BINDING SITE III. Table 2 illustrates the complementarity of the nucleotide sequences of KIAA1737 BINDING SITE, designated SEQ ID:2789, to the nucleotide sequence of VGAM170 RNA, herein designated VGAM RNA, also designated SEQ ID:505.

Another function of VGAM170 is therefore inhibition of KIAA1737 (Accession XM_{—}041115). Accordingly, utilities of VGAM170 include diagnosis, prevention and treatment of diseases and clinical conditions associated with KIAA1737. KIAA1750 (Accession XM_{—}043067) is another VGAM170 host target gene. KIAA1750 BINDING SITE is HOST TARGET binding site found in the 3′ untranslated region of mRNA encoded by KIAA1750, corresponding to a HOST TARGET binding site such as BINDING SITE I, BINDING SITE II or BINDING SITE III. Table 2 illustrates the complementarity of the nucleotide sequences of KIAA1750 BINDING SITE, designated SEQ ID:2811, to the nucleotide sequence of VGAM170 RNA, herein designated VGAM RNA, also designated SEQ ID:505.

Another function of VGAM170 is therefore inhibition of KIAA1750 (Accession XM_{—}043067). Accordingly, utilities of VGAM170 include diagnosis, prevention and treatment of diseases and clinical conditions associated with KIAA1750. KIAA1900 (Accession XM_{—}055299) is another VGAM170 host target gene. KIAA1900 BINDING SITE is HOST TARGET binding site found in the 3′ untranslated region of mRNA encoded by KIAA1900, corresponding to a HOST TARGET binding site such as BINDING SITE I, BINDING SITE II or BINDING SITE III. Table 2 illustrates the complementarity of the nucleotide sequences of KIAA1900 BINDING SITE, designated SEQ ID:2972, to the nucleotide sequence of VGAM170 RNA, herein designated VGAM RNA, also designated SEQ ID:505.

Another function of VGAM170 is therefore inhibition of KIAA1900 (Accession XM_{—}055299). Accordingly, utilities of VGAM170 include diagnosis, prevention and treatment of diseases and clinical conditions associated with KIAA1900. KIAA1946 (Accession XM_{—}092459) is another VGAM170 host target gene. KIAA1946 BINDING SITE is HOST TARGET binding site found in the 3′ untranslated region of mRNA encoded by KIAA1946, corresponding to a HOST TARGET binding site such as BINDING SITE I, BINDING SITE II or BINDING SITE III. Table 2 illustrates the complementarity of the nucleotide sequences of KIAA1946 BINDING SITE, designated SEQ ID:3246, to the nucleotide sequence of VGAM170 RNA, herein designated VGAM RNA, also designated SEQ ID:505.

Another function of VGAM170 is therefore inhibition of KIAA1946 (Accession XM_{—}092459). Accordingly, utilities of VGAM170 include diagnosis, prevention and treatment of diseases and clinical conditions associated with KIAA1946. LALP1 (Accession NM_{—}020354) is another VGAM170 host target gene. LALP1 BINDING SITE is HOST TARGET binding site found in the 3′ untranslated region of mRNA encoded by LALP1, corresponding to a HOST TARGET binding site such as BINDING SITE I, BINDING SITE II or BINDING SITE III. Table 2 illustrates the complementarity of the nucleotide sequences of LALP1 BINDING SITE, designated SEQ ID:1915, to the nucleotide sequence of VGAM170 RNA, herein designated VGAM RNA, also designated SEQ ID:505.

Another function of VGAM170 is therefore inhibition of LALP1 (Accession NM_{—}020354). Accordingly, utilities of VGAM170 include diagnosis, prevention and treatment of diseases and clinical conditions associated with LALP1. Leucine Rich Repeat (in FLII) Interacting Protein 2 (LRRFIP2, Accession NM_{—}017724) is another VGAM170 host target gene. LRRFIP2 BINDING SITE is HOST TARGET binding site found in the 3′ untranslated region of mRNA encoded by LRRFIP2, corresponding to a HOST TARGET binding site such as BINDING SITE I, BINDING SITE II or BINDING SITE III. Table 2 illustrates the complementarity of the nucleotide sequences of LRRFIP2 BINDING SITE, designated SEQ ID:1743, to the nucleotide sequence of VGAM170 RNA, herein designated VGAM RNA, also designated SEQ ID:505.

Another function of VGAM170 is therefore inhibition of Leucine Rich Repeat (in FLII) Interacting Protein 2 (LRRFIP2, Accession NM_{—}017724). Accordingly, utilities of VGAM170 include diagnosis, prevention and treatment of diseases and clinical conditions associated with LRRFIP2. MGC1842 (Accession XM_{—}037797) is another VGAM170 host target gene. MGC1842 BINDING SITE is HOST TARGET binding site found in the 3′ untranslated region of mRNA encoded by MGC1842, corresponding to a HOST TARGET binding site such as BINDING SITE I, BINDING SITE II or BINDING SITE III. Table 2 illustrates the complementarity of the nucleotide sequences of MGC1842 BINDING SITE, designated SEQ ID:2724, to the nucleotide sequence of VGAM170 RNA, herein designated VGAM RNA, also designated SEQ ID:505.

Another function of VGAM170 is therefore inhibition of MGC1842 (Accession XM_{—}037797). Accordingly, utilities of VGAM170 include diagnosis, prevention and treatment of diseases and clinical conditions associated with MGC1842. MGC9753 (Accession NM_{—}033419) is another VGAM170 host target gene. MGC9753 BINDING SITE is HOST TARGET binding site found in the 5′ untranslated region of mRNA encoded by MGC9753, corresponding to a HOST TARGET binding site such as BINDING SITE I, BINDING SITE II or BINDING SITE III. Table 2 illustrates the complementarity of the nucleotide sequences of MGC9753 BINDING SITE, designated SEQ ID:2333, to the nucleotide sequence of VGAM170 RNA, herein designated VGAM RNA, also designated SEQ ID:505.

Another function of VGAM170 is therefore inhibition of MGC9753 (Accession NM_{—}033419). Accordingly, utilities of VGAM170 include diagnosis, prevention and treatment of diseases and clinical conditions associated with MGC9753. Paternally Expressed 10 (PEG10, Accession NM_{—}015068) is another VGAM170 host target gene. PEG10 BINDING SITE is HOST TARGET binding site found in the 3′ untranslated region of mRNA encoded by PEG10, corresponding to a HOST TARGET binding site such as BINDING SITE I, BINDING SITE II or BINDING SITE III. Table 2 illustrates the complementarity of the nucleotide sequences of PEG10 BINDING SITE, designated SEQ ID:1606, to the nucleotide sequence of VGAM170 RNA, herein designated VGAM RNA, also designated SEQ ID:505.

Another function of VGAM170 is therefore inhibition of Paternally Expressed 10 (PEG10, Accession NM_{—}015068). Accordingly, utilities of VGAM170 include diagnosis, prevention and treatment of diseases and clinical conditions associated with PEG10. Progesterone Receptor Membrane Component 2 (PGRMC2, Accession NM_{—}006320) is another VGAM170 host target gene. PGRMC2 BINDING SITE is HOST TARGET binding site found in the 3′ untranslated region of mRNA encoded by PGRMC2, corresponding to a HOST TARGET binding site such as BINDING SITE I, BINDING SITE II or BINDING SITE III. Table 2 illustrates the complementarity of the nucleotide sequences of PGRMC2 BINDING SITE, designated SEQ ID:1287, to the nucleotide sequence of VGAM170 RNA, herein designated VGAM RNA, also designated SEQ ID:505.

Another function of VGAM170 is therefore inhibition of Progesterone Receptor Membrane Component 2 (PGRMC2, Accession NM_{—}006320). Accordingly, utilities of VGAM170 include diagnosis, prevention and treatment of diseases and clinical conditions associated with PGRMC2. Protein Phosphatase 4, Regulatory Subunit 1-like (PPP4R1L, Accession XM_{—}086650) is another VGAM170 host target gene. PPP4R1L BINDING SITE is HOST TARGET binding site found in the 5′ untranslated region of mRNA encoded by PPP4R1L, corresponding to a HOST TARGET binding site such as BINDING SITE I, BINDING SITE II or BINDING SITE III. Table 2 illustrates the complementarity of the nucleotide sequences of PPP4R1L BINDING SITE, designated SEQ ID:3139, to the nucleotide sequence of VGAM170 RNA, herein designated VGAM RNA, also designated SEQ ID:505.

Another function of VGAM170 is therefore inhibition of Protein Phosphatase 4, Regulatory Subunit 1-like (PPP4R1L, Accession XM_{—}086650). Accordingly, utilities of VGAM170 include diagnosis, prevention and treatment of diseases and clinical conditions associated with PPP4R1L. PRO0386 (Accession NM_{—}018562) is another VGAM170 host target gene. PRO0386 BINDING SITE is HOST TARGET binding site found in the 5′ untranslated region of mRNA encoded by PRO0386, corresponding to a HOST TARGET binding site such as BINDING SITE I, BINDING SITE II or BINDING SITE III. Table 2 illustrates the complementarity of the nucleotide sequences of PRO0386 BINDING SITE, designated SEQ ID:1843, to the nucleotide sequence of VGAM170 RNA, herein designated VGAM RNA, also designated SEQ ID:505.

Another function of VGAM170 is therefore inhibition of PRO0386 (Accession NM_{—}018562). Accordingly, utilities of VGAM170 include diagnosis, prevention and treatment of diseases and clinical conditions associated with PRO0386. PRO0456 (Accession NM_{—}014127) is another VGAM170 host target gene. PRO0456 BINDING SITE is HOST TARGET binding site found in the 3′ untranslated region of mRNA encoded by PRO0456, corresponding to a HOST TARGET binding site such as BINDING SITE I, BINDING SITE II or BINDING SITE III. Table 2 illustrates the complementarity of the nucleotide sequences of PRO0456 BINDING SITE, designated SEQ ID: 1476, to the nucleotide sequence of VGAM170 RNA, herein designated VGAM RNA, also designated SEQ ID:505.

Another function of VGAM170 is therefore inhibition of PRO0456 (Accession NM_{—}014127). Accordingly, utilities of VGAM170 include diagnosis, prevention and treatment of diseases and clinical conditions associated with PRO0456. Solute Carrier Family 26, Member 8 (SLC26A8, Accession NM_{—}138718) is another VGAM170 host target gene. SLC26A8 BINDING SITE is HOST TARGET binding site found in the 5′ untranslated region of mRNA encoded by SLC26A8, corresponding to a HOST TARGET binding site such as BINDING SITE I, BINDING SITE II or BINDING SITE III. Table 2 illustrates the complementarity of the nucleotide sequences of SLC26A8 BINDING SITE, designated SEQ ID:2455, to the nucleotide sequence of VGAM170 RNA, herein designated VGAM RNA, also designated SEQ ID:505.

Another function of VGAM170 is therefore inhibition of Solute Carrier Family 26, Member 8 (SLC26A8, Accession NM_{—}138718). Accordingly, utilities of VGAM170 include diagnosis, prevention and treatment of diseases and clinical conditions associated with SLC26A8. Syntrophin, Gamma 1 (SNTG1, Accession NM_{—}018967) is another VGAM170 host target gene. SNTG1 BINDING SITE is HOST TARGET binding site found in the 5′ untranslated region of mRNA encoded by SNTG1, corresponding to a HOST TARGET binding site such as BINDING SITE I, BINDING SITE II or BINDING SITE III. Table 2 illustrates the complementarity of the nucleotide sequences of SNTG1 BINDING SITE, designated SEQ ID:1867, to the nucleotide sequence of VGAM170 RNA, herein designated VGAM RNA, also designated SEQ ID:505.

Another function of VGAM170 is therefore inhibition of Syntrophin, Gamma 1 (SNTG1, Accession NM_{—}018967). Accordingly, utilities of VGAM170 include diagnosis, prevention and treatment of diseases and clinical conditions associated with SNTG1. Zinc Finger Protein 396 (ZNF396, Accession XM_{—}064632) is another VGAM170 host target gene. ZNF396 BINDING SITE is HOST TARGET binding site found in the 3′ untranslated region of mRNA encoded by ZNF396, corresponding to a HOST TARGET binding site such as BINDING SITE I, BINDING SITE II or BINDING SITE III. Table 2 illustrates the complementarity of the nucleotide sequences of ZNF396 BINDING SITE, designated SEQ ID:3034, to the nucleotide sequence of VGAM170 RNA, herein designated VGAM RNA, also designated SEQ ID:505.

Another function of VGAM170 is therefore inhibition of Zinc Finger Protein 396 (ZNF396, Accession XM_{—}064632). Accordingly, utilities of VGAM170 include diagnosis, prevention and treatment of diseases and clinical conditions associated with ZNF396. LOC145098 (Accession XM_{—}085022) is another VGAM170 host target gene. LOC145098 BINDING SITE is HOST TARGET binding site found in the 5′ untranslated region of mRNA encoded by LOC145098, corresponding to a HOST TARGET binding site such as BINDING SITE I, BINDING SITE II or BINDING SITE III. Table 2 illustrates the complementarity of the nucleotide sequences of LOC145098 BINDING SITE, designated SEQ ID:3074, to the nucleotide sequence of VGAM170 RNA, herein designated VGAM RNA, also designated SEQ ID:505.

Another function of VGAM170 is therefore inhibition of LOC145098 (Accession XM_{—}085022). Accordingly, utilities of VGAM170 include diagnosis, prevention and treatment of diseases and clinical conditions associated with LOC145098. LOC146136 (Accession XM_{—}053737) is another VGAM170 host target gene. LOC146136 BINDING SITE is HOST TARGET binding site found in the 5′ untranslated region of mRNA encoded by LOC146136, corresponding to a HOST TARGET binding site such as BINDING SITE I, BINDING SITE II or BINDING SITE III. Table 2 illustrates the complementarity of the nucleotide sequences of LOC146136 BINDING SITE, designated SEQ ID:2964, to the nucleotide sequence of VGAM170 RNA, herein designated VGAM RNA, also designated SEQ ID:505.

Another function of VGAM170 is therefore inhibition of LOC146136 (Accession XM_{—}053737). Accordingly, utilities of VGAM170 include diagnosis, prevention and treatment of diseases and clinical conditions associated with LOC146136. LOC148195 (Accession XM_{—}097419) is another VGAM170 host target gene. LOC148195 BINDING SITE is HOST TARGET binding site found in the 3′ untranslated region of mRNA encoded by LOC148195, corresponding to a HOST TARGET binding site such as BINDING SITE I, BINDING SITE II or BINDING SITE III. Table 2 illustrates the complementarity of the nucleotide sequences of LOC148195 BINDING SITE, designated SEQ ID:3298, to the nucleotide sequence of VGAM170 RNA, herein designated VGAM RNA, also designated SEQ ID:505.

Another function of VGAM170 is therefore inhibition of LOC148195 (Accession XM_{—}097419). Accordingly, utilities of VGAM170 include diagnosis, prevention and treatment of diseases and clinical conditions associated with LOC148195. LOC149086 (Accession XM_{—}097580) is another VGAM170 host target gene. LOC149086 BINDING SITE is HOST TARGET binding site found in the 5′ untranslated region of mRNA encoded by LOC149086, corresponding to a HOST TARGET binding site such as BINDING SITE I, BINDING SITE II or BINDING SITE III. Table 2 illustrates the complementarity of the nucleotide sequences of LOC149086 BINDING SITE, designated SEQ ID:3306, to the nucleotide sequence of VGAM170 RNA, herein designated VGAM RNA, also designated SEQ ID:505.

Another function of VGAM170 is therefore inhibition of LOC149086 (Accession XM_{—}097580). Accordingly, utilities of VGAM170 include diagnosis, prevention and treatment of diseases and clinical conditions associated with LOC149086. LOC149420 (Accession XM_{—}086530) is another VGAM170 host target gene. LOC149420 BINDING SITE is HOST TARGET binding site found in the 3′ untranslated region of mRNA encoded by LOC149420, corresponding to a HOST TARGET binding site such as BINDING SITE I, BINDING SITE II or BINDING SITE III. Table 2 illustrates the complementarity of the nucleotide sequences of LOC149420 BINDING SITE, designated SEQ ID:3133, to the nucleotide sequence of VGAM170 RNA, herein designated VGAM RNA, also designated SEQ ID:505.

Another function of VGAM170 is therefore inhibition of LOC149420 (Accession XM_{—}086530). Accordingly, utilities of VGAM170 include diagnosis, prevention and treatment of diseases and clinical conditions associated with LOC149420. LOC149912 (Accession XM_{—}097743) is another VGAM170 host target gene. LOC149912 BINDING SITE is HOST TARGET binding site found in the 5′ untranslated region of mRNA encoded by LOC149912, corresponding to a HOST TARGET binding site such as BINDING SITE I, BINDING SITE II or BINDING SITE III. Table 2 illustrates the complementarity of the nucleotide sequences of LOC149912 BINDING SITE, designated SEQ ID:3317, to the nucleotide sequence of VGAM170 RNA, herein designated VGAM RNA, also designated SEQ ID:505.

Another function of VGAM170 is therefore inhibition of LOC149912 (Accession XM_{—}097743). Accordingly, utilities of VGAM170 include diagnosis, prevention and treatment of diseases and clinical conditions associated with LOC149912. LOC151816 (Accession XM_{—}098122) is another VGAM170 host target gene. LOC151816 BINDING SITE is HOST TARGET binding site found in the 5′ untranslated region of mRNA encoded by LOC151816, corresponding to a HOST TARGET binding site such as BINDING SITE I, BINDING SITE II or BINDING SITE III. Table 2 illustrates the complementarity of the nucleotide sequences of LOC151816 BINDING SITE, designated SEQ ID:3350, to the nucleotide sequence of VGAM170 RNA, herein designated VGAM RNA, also designated SEQ ID:505.

Another function of VGAM170 is therefore inhibition of LOC151816 (Accession XM_{—}098122). Accordingly, utilities of VGAM170 include diagnosis, prevention and treatment of diseases and clinical conditions associated with LOC151816. LOC152991 (Accession XM_{—}098295) is another VGAM170 host target gene. LOC152991 BINDING SITE is HOST TARGET binding site found in the 5′ untranslated region of mRNA encoded by LOC152991, corresponding to a HOST TARGET binding site such as BINDING SITE I, BINDING SITE II or BINDING SITE III. Table 2 illustrates the complementarity of the nucleotide sequences of LOC152991 BINDING SITE, designated SEQ ID:3365, to the nucleotide sequence of VGAM170 RNA, herein designated VGAM RNA, also designated SEQ ID:505.

Another function of VGAM170 is therefore inhibition of LOC152991 (Accession XM_{—}098295). Accordingly, utilities of VGAM170 include diagnosis, prevention and treatment of diseases and clinical conditions associated with LOC152991. LOC157556 (Accession XM_{—}098783) is another VGAM170 host target gene. LOC157556 BINDING SITE is HOST TARGET binding site found in the 3′ untranslated region of mRNA encoded by LOC157556, corresponding to a HOST TARGET binding site such as BINDING SITE I, BINDING SITE II or BINDING SITE III. Table 2 illustrates the complementarity of the nucleotide sequences of LOC157556 BINDING SITE, designated SEQ ID:3382, to the nucleotide sequence of VGAM170 RNA, herein designated VGAM RNA, also designated SEQ ID:505.

Another function of VGAM170 is therefore inhibition of LOC157556 (Accession XM_{—}098783). Accordingly, utilities of VGAM170 include diagnosis, prevention and treatment of diseases and clinical conditions associated with LOC157556. LOC157697 (Accession XM_{—}088365) is another VGAM170 host target gene. LOC157697 BINDING SITE is HOST TARGET binding site found in the 5′ untranslated region of mRNA encoded by LOC157697, corresponding to a HOST TARGET binding site such as BINDING SITE I, BINDING SITE II or BINDING SITE III. Table 2 illustrates the complementarity of the nucleotide sequences of LOC157697 BINDING SITE, designated SEQ ID:3204, to the nucleotide sequence of VGAM170 RNA, herein designated VGAM RNA, also designated SEQ ID:505.

Another function of VGAM170 is therefore inhibition of LOC157697 (Accession XM_{—}088365). Accordingly, utilities of VGAM170 include diagnosis, prevention and treatment of diseases and clinical conditions associated with LOC157697. LOC158431 (Accession XM_{—}098940) is another VGAM170 host target gene. LOC158431 BINDING SITE is HOST TARGET binding site found in the 3′ untranslated region of mRNA encoded by LOC158431, corresponding to a HOST TARGET binding site such as BINDING SITE I, BINDING SITE II or BINDING SITE III. Table 2 illustrates the complementarity of the nucleotide sequences of LOC158431 BINDING SITE, designated SEQ ID:3390, to the nucleotide sequence of VGAM170 RNA, herein designated VGAM RNA, also designated SEQ ID:505.

Another function of VGAM170 is therefore inhibition of LOC158431 (Accession XM_{—}098940). Accordingly, utilities of VGAM170 include diagnosis, prevention and treatment of diseases and clinical conditions associated with LOC158431. LOC200488 (Accession XM_{—}117240) is another VGAM170 host target gene. LOC200488 BINDING SITE is HOST TARGET binding site found in the 5′ untranslated region of mRNA encoded by LOC200488, corresponding to a HOST TARGET binding site such as BINDING SITE I, BINDING SITE II or BINDING SITE III. Table 2 illustrates the complementarity of the nucleotide sequences of LOC200488 BINDING SITE, designated SEQ ID:3478, to the nucleotide sequence of VGAM170 RNA, herein designated VGAM RNA, also designated SEQ ID:505.

Another function of VGAM170 is therefore inhibition of LOC200488 (Accession XM_{—}117240). Accordingly, utilities of VGAM170 include diagnosis, prevention and treatment of diseases and clinical conditions associated with LOC200488. LOC220565 (Accession XM_{—}165417) is another VGAM170 host target gene. LOC220565 BINDING SITE is HOST TARGET binding site found in the 3′ untranslated region of mRNA encoded by LOC220565, corresponding to a HOST TARGET binding site such as BINDING SITE I, BINDING SITE II or BINDING SITE III. Table 2 illustrates the complementarity of the nucleotide sequences of LOC220565 BINDING SITE, designated SEQ ID:3494, to the nucleotide sequence of VGAM170 RNA, herein designated VGAM RNA, also designated SEQ ID:505.

Another function of VGAM170 is therefore inhibition of LOC220565 (Accession XM_{—}165417). Accordingly, utilities of VGAM170 include diagnosis, prevention and treatment of diseases and clinical conditions associated with LOC220565. LOC221751 (Accession XM_{—}166370) is another VGAM170 host target gene. LOC221751 BINDING SITE is HOST TARGET binding site found in the 5′ untranslated region of mRNA encoded by LOC221751, corresponding to a HOST TARGET binding site such as BINDING SITE I, BINDING SITE II or BINDING SITE III. Table 2 illustrates the complementarity of the nucleotide sequences of LOC221751 BINDING SITE, designated SEQ ID:3552, to the nucleotide sequence of VGAM170 RNA, herein designated VGAM RNA, also designated SEQ ID:505.

Another function of VGAM170 is therefore inhibition of LOC221751 (Accession XM_{—}166370). Accordingly, utilities of VGAM170 include diagnosis, prevention and treatment of diseases and clinical conditions associated with LOC221751. LOC253978 (Accession XM_{—}174851) is another VGAM170 host target gene. LOC253978 BINDING SITE is HOST TARGET binding site found in the 5′ untranslated region of mRNA encoded by LOC253978, corresponding to a HOST TARGET binding site such as BINDING SITE I, BINDING SITE II or BINDING SITE III. Table 2 illustrates the complementarity of the nucleotide sequences of LOC253978 BINDING SITE, designated SEQ ID:3743, to the nucleotide sequence of VGAM170 RNA, herein designated VGAM RNA, also designated SEQ ID:505.

Another function of VGAM170 is therefore inhibition of LOC253978 (Accession XM_{—}174851). Accordingly, utilities of VGAM170 include diagnosis, prevention and treatment of diseases and clinical conditions associated with LOC253978. LOC257319 (Accession XM_{—}171049) is another VGAM170 host target gene. LOC257319 BINDING SITE is HOST TARGET binding site found in the 3′ untranslated region of mRNA encoded by LOC257319, corresponding to a HOST TARGET binding site such as BINDING SITE I, BINDING SITE II or BINDING SITE III. Table 2 illustrates the complementarity of the nucleotide sequences of LOC257319 BINDING SITE, designated SEQ ID:3683, to the nucleotide sequence of VGAM170 RNA, herein designated VGAM RNA, also designated SEQ ID:505.

Another function of VGAM170 is therefore inhibition of LOC257319 (Accession XM_{—}171049). Accordingly, utilities of VGAM170 include diagnosis, prevention and treatment of diseases and clinical conditions associated with LOC257319. LOC92162 (Accession XM_{—}043273) is another VGAM170 host target gene. LOC92162 BINDING SITE is HOST TARGET binding site found in the 5′ untranslated region of mRNA encoded by LOC92162, corresponding to a HOST TARGET binding site such as BINDING SITE I, BINDING SITE II or BINDING SITE III. Table 2 illustrates the complementarity of the nucleotide sequences of LOC92162 BINDING SITE, designated SEQ ID:2816, to the nucleotide sequence of VGAM170 RNA, herein designated VGAM RNA, also designated SEQ ID:505.

Another function of VGAM170 is therefore inhibition of LOC92162 (Accession XM_{—}043273). Accordingly, utilities of VGAM170 include diagnosis, prevention and treatment of diseases and clinical conditions associated with LOC92162. LOC92391 (Accession XM_{—}044793) is another VGAM170 host target gene. LOC92391 BINDING SITE is HOST TARGET binding site found in the 3′ untranslated region of mRNA encoded by LOC92391, corresponding to a HOST TARGET binding site such as BINDING SITE I, BINDING SITE II or BINDING SITE III. Table 2 illustrates the complementarity of the nucleotide sequences of LOC92391 BINDING SITE, designated SEQ ID:2842, to the nucleotide sequence of VGAM170 RNA, herein designated VGAM RNA, also designated SEQ ID:505.

Another function of VGAM170 is therefore inhibition of LOC92391 (Accession XM_{—}044793). Accordingly, utilities of VGAM170 include diagnosis, prevention and treatment of diseases and clinical conditions associated with LOC92391. FIG. 1 further provides a conceptual description of a novel bioinformatically detected viral gene of the present invention, referred to here as Viral Genomic Address Messenger 171 (VGAM171) viral gene, which modulates expression of respective host target genes thereof, the function and utility of which host target genes is known in the art.

VGAM171 is a novel bioinformatically detected regulatory, non protein coding, viral micro RNA (miRNA) gene. The method by which VGAM171 was detected is described hereinabove with reference to FIGS. 1-8.

VGAM171 gene, herein designated VGAM GENE, is a viral gene contained in the genome of Vaccinia Virus. VGAM171 host target gene, herein designated VGAM HOST TARGET GENE, is a human gene contained in the human genome.

VGAM171 gene encodes a VGAM171 precursor RNA, herein designated VGAM PRECURSOR RNA. Similar to other miRNA genes, and unlike most ordinary genes, VGAM171 precursor RNA does not encode a protein. A nucleotide sequence identical or highly similar to the nucleotide sequence of VGAM171 precursor RNA is designated SEQ ID: 157, and is provided hereinbelow with reference to the sequence listing part. Nucleotide sequence SEQ ID:157 is located at position 90530 relative to the genome of Vaccinia Virus.

VGAM171 precursor RNA folds onto itself, forming VGAM171 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM171 folded precursor RNA into VGAM171 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM171 RNA is designated SEQ ID:506, and is provided hereinbelow with reference to the sequence listing part.

VGAM171 host target gene, herein designated VGAM HOST TARGET GENE, encodes a corresponding messenger RNA, VGAM171 host target RNA, herein designated VGAM HOST TARGET RNA. VGAM171 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM171 RNA, herein designated VGAM RNA, binds complementarily to one or more host target binding sites located in untranslated regions of VGAM171 host target RNA, herein designated VGAM HOST TARGET RNA. This complementary binding is due to the fact that the nucleotide sequence of VGAM171 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM171 RNA, herein designated VGAM RNA, may have a different number of host target binding sites in untranslated regions of a VGAM171 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM171 RNA, herein designated VGAM RNA, to host target binding sites on VGAM171 host target RNA, herein designated VGAM HOST TARGET RNA, such as BINDING SITE I, BINDING SITE II and BINDING SITE III, inhibits translation of VGAM171 host target RNA into VGAM171 host target protein, herein designated VGAM HOST TARGET PROTEIN. VGAM host target protein is therefore outlined by a broken line.

It is appreciated that VGAM171 host target gene, herein designated VGAM HOST TARGET GENE, in fact represents a plurality of VGAM171 host target genes. The mRNA of each one of this plurality of VGAM171 host target genes comprises one or more host target binding sites, each having a nucleotide sequence which is at least partly complementary to VGAM171 RNA, herein designated VGAM RNA, and which when bound by VGAM171 RNA causes inhibition of translation of respective one or more VGAM171 host target proteins.

It is further appreciated by one skilled in the art that the mode of translational inhibition illustrated by FIG. 1 with specific reference to translational inhibition exerted by VGAM171 gene, herein designated VGAM GENE, on one or more VGAM171 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM171 is inhibition of expression of host target genes, as part of a novel viral mechanism of attacking a host. Accordingly, utilities of VGAM171 include diagnosis, prevention and treatment of viral infection by Vaccinia Virus. Specific functions, and accordingly utilities, of VGAM171 correlate with, and may be deduced from, the identity of the host target genes which VGAM171 binds and inhibits, and the function of these host target genes, as elaborated hereinbelow.

Nucleotide sequences of the VGAM171 precursor RNA, herein designated VGAM PRECURSOR RNA, and of the ‘diced’ VGAM171 RNA, herein designated VGAM RNA, and a schematic representation of the secondary folding of VGAM171 folded precursor RNA, herein designated VGAM FOLDED PRECURSOR RNA, of VGAM171 are further described hereinbelow with reference to Table 1.

Nucleotide sequences of host target binding sites, such as BINDING SITE-I, BINDING SITE-II and BINDING SITE-III of FIG. 1, found on VGAM171 host target RNA, and schematic representation of the complementarity of each of these host target binding sites to VGAM171 RNA, herein designated VGAM RNA, are described hereinbelow with reference to Table 2.

As mentioned hereinabove with reference to FIG. 1, a function of VGAM171 gene, herein designated VGAM is inhibition of expression of VGAM171 target genes. It is appreciated that specific functions, and accordingly utilities, of VGAM171 correlate with, and may be deduced from, the identity of the target genes which VGAM171 binds and inhibits, and the function of these target genes, as elaborated hereinbelow.

BTB and CNC Homology 1, Basic Leucine Zipper Transcription Factor 2 (BACH2, Accession NM_{—}021813) is a VGAM171 host target gene. BACH2 BINDING SITE is HOST TARGET binding site found in the 3′ untranslated region of mRNA encoded by BACH2, corresponding to a HOST TARGET binding site such as BINDING SITE I, BINDING SITE II or BINDING SITE III. Table 2 illustrates the complementarity of the nucleotide sequences of BACH2 BINDING SITE, designated SEQ ID:1963, to the nucleotide sequence of VGAM171 RNA, herein designated VGAM RNA, also designated SEQ ID:506.

A function of VGAM171 is therefore inhibition of BTB and CNC Homology 1, Basic Leucine Zipper Transcription Factor 2 (BACH2, Accession NM_{—}021813), a gene which acts as repressor or activator, binds to maf recognition elements. Accordingly, utilities of VGAM171 include diagnosis, prevention and treatment of diseases and clinical conditions associated with BACH2. The function of BACH2 and its association with various diseases and clinical conditions, has been established by previous studies, as described hereinabove with reference to VGAM119. Nucleolin (NCL, Accession XM_{—}010858) is another VGAM171 host target gene. NCL BINDING SITE is HOST TARGET binding site found in the 3′ untranslated region of mRNA encoded by NCL, corresponding to a HOST TARGET binding site such as BINDING SITE I, BINDING SITE II or BINDING SITE III. Table 2 illustrates the complementarity of the nucleotide sequences of NCL BINDING SITE, designated SEQ ID:2552, to the nucleotide sequence of VGAM171 RNA, herein designated VGAM RNA, also designated SEQ ID:506.

Another function of VGAM171 is therefore inhibition of Nucleolin (NCL, Accession XM_{—}010858), a gene which induces chromatin decondensation by binding to histone h1. Accordingly, utilities of VGAM171 include diagnosis, prevention and treatment of diseases and clinical conditions associated with NCL. The function of NCL has been established by previous studies. Nucleolin, also known as C23, is an abundantly expressed acidic phosphoprotein of exponentially growing cells and is located mainly in dense fibrillar regions of the nucleolus. Rt is involved in the control of transcription of ribosomal RNA (rRNA) genes by RNA polymerase I, in ribosome maturation and assembly, and in nucleocytoplasmic transportation of ribosomal components. From a lambda-gt10 human retinal library using a bovine cDNA probe, Srivastava et al. (1989) isolated a cDNA containing the entire coding region of nucleolin. The cDNA hybridized to a transcript of 3,000 bases from fast-dividing cells, as well as terminally differentiated tissues of several species. Translation of the nucleotide sequence showed a long open reading frame which predicted a 707-amino acid protein with several distinct domains. Srivastava et al. (1990) found that the gene consists of 14 exons with 13 introns and spans approximately 11 kb. Sequences in the 5-prime flanking region and the first intron contain a high content of GC residues consistent with nucleolin being a housekeeping gene. Southern blot analysis indicated that the gene is present in 1 copy in the haploid genome. Nicoloso et al. (1994) demonstrated that intron 11 of the NCL gene in humans and rodents encodes a small nucleolar RNA, which they designated U20 (OMIM Ref. No. 604012). Sequence analysis revealed that U20 contains a region of perfect complementarity with a conserved sequence in 18S rRNA, suggesting that like nucleolin, it may be involved in the formation of the small ribosomal subunit

Full details of the abovementioned studies are described in the following publications, the disclosure of which are hereby incorporated by reference:
Nicoloso, M.; Caizergues-Ferrer, M.; Michot, B.; Azum, M.-C.; Bachellerie, J.-P.: U20, a novel small nucleolar RNA, is encoded in an intron of the nucleolin gene in mammals. Molec. Cell. Biol. 14: 5766-5776, 1994.; and
Srivastava, M.; Fleming, P. J.; Pollard, H. B.; Burns, A. L.: Cloning and sequencing of the human nucleolin cDNA. FEBS Lett. 250: 99-105, 1989.

Further studies establishing the function and utilities of NCL are found in John Hopkins OMIM database record ID 164035, and in sited publications numbered 363-365 listed in the bibliography section hereinbelow, which are also hereby incorporated by reference. Neogenin Homolog 1 (chicken) (NEO1, Accession NM_{—}002499) is another VGAM171 host target gene. NEO1 BINDING SITE is HOST TARGET binding site found in the 3′ untranslated region of mRNA encoded by NEO1, corresponding to a HOST TARGET binding site such as BINDING SITE I, BINDING SITE II or BINDING SITE III. Table 2 illustrates the complementarity of the nucleotide sequences of NEO1 BINDING SITE, designated SEQ ID:932, to the nucleotide sequence of VGAM171 RNA, herein designated VGAM RNA, also designated SEQ ID:506.

Another function of VGAM171 is therefore inhibition of Neogenin Homolog 1 (chicken) (NEO1, Accession NM_{—}002499), a gene which regulates the transition of undifferentiated proliferating cells to their differentiated state. Accordingly, utilities of VGAM171 include diagnosis, prevention and treatment of diseases and clinical conditions associated with NEO1. The function of NEO1 has been established by previous studies. Vielmetter et al. (1994) identified a protein with roughly 50% amino acid identity to DCC (OMIM Ref. No. 120470); it showed a dynamic pattern of expression in the developing nervous system and gastrointestinal tract of the chicken. They termed this protein neogenin. Specifically, neogenin was induced in neural cells immediately before cell cycle withdrawal and terminal differentiation. Meyerhardt et al. (1997) cloned the human neogenin gene (symbolized NGN by them) and explored its possible role in cancer. They found cDNAs for 2 alternatively spliced forms of NGN, encoding proteins of 1,461 and 1,408 amino acids. By fluorescence in situ hybridization (FISH) they localized NGN in 15q22, a region infrequently affected by alterations in cancer. NGN transcripts of about 7.5 and 5.5 kb were detected in all adult tissues studied. In contrast to the frequent loss of DCC expression in cancers, no alterations in NGN expression were observed in more than 50 cancers studied, including glioblastoma, medulloblastoma, neuroblastoma, colorectal, breast, cervical, and pancreatic cancer cell lines, and xenografts. Based on their sequence conservation and similar expression during development, Meyerhardt et al. (1997) concluded that DCC and NGN may have related functions; however, the chromosomal location and ubiquitous expression of NGN in various human tumors suggested it is infrequently altered in cancer. Vielmetter et al. (1997) also cloned and characterized human neogenin, and symbolized the gene NEO1. They mapped NEO1 to 15q22.3-q23 by FISH.

Full details of the abovementioned studies are described in the following publications, the disclosure of which are hereby incorporated by reference:
Meyerhardt, J. A.; Look, A. T.; Bigner, S. H.; Fearon, E. R.: Identification and characterization of neogenin, a DCC-related gene. Oncogene 14: 1129-1136, 1997.; and
Vielmetter, J.; Kayyem, J. F.; Roman, J. M.; Dreyer, W. J.: Neogenin, an avian cell surface protein expressed during terminal neuronal differentiation, is closely related to the human.

Further studies establishing the function and utilities of NEO1 are found in John Hopkins OMIM database record ID 601907, and in sited publications numbered 2004-2006 listed in the bibliography section hereinbelow, which are also hereby incorporated by reference. Solute Carrier Family 21 (organic anion transporter), Member 3 (SLC21A3, Accession NM_{—}005075) is another VGAM171 host target gene. SLC21A3 BINDING SITE is HOST TARGET binding site found in the 3′ untranslated region of mRNA encoded by SLC21A3, corresponding to a HOST TARGET binding site such as BINDING SITE I, BINDING SITE II or BINDING SITE III. Table 2 illustrates the complementarity of the nucleotide sequences of SLC21A3 BINDING SITE, designated SEQ ID:1183, to the nucleotide sequence of VGAM171 RNA, herein designated VGAM RNA, also designated SEQ ID:506.

Another function of VGAM171 is therefore inhibition of Solute Carrier Family 21 (organic anion transporter), Member 3 (SLC21A3, Accession NM_{—}005075), a gene which mediates the na(+)-independent transport of organic anions such as bsp and conjugated (taurocholate) and unconjugated (cholate) bile acids. Accordingly, utilities of VGAM171 include diagnosis, prevention and treatment of diseases and clinical conditions associated with SLC21A3. The function of SLC21A3 has been established by previous studies. The organic anion transporter (OATP) of liver mediates the basolateral hepatocellular uptake of numerous cholephilic anions and steroidal compounds from sinusoidal blood. By screening a human liver cDNA library with a rat Oatp cDNA, Kullak-Ublick et al. (1995) cloned a cDNA encoding OATP. The deduced 670-amino acid OATP protein has 12 putative transmembrane domains and 8 potential N-linked glycosylation sites. The human and rat OATP proteins are 67% identical. In vitro translation produced unglycosylated and glycosylated human OATP proteins that migrated as 59-kD and 71-kD polypeptides, respectively, in SDS-polyacrylamide gels. Functional studies in Xenopus oocytes showed that OATP mediates sodium-independent transport of the xenobiotic bromosulfophthalein and of endogenous conjugated and unconjugated bile acids. Northern blot analysis detected an approximately 2.7-kb OATP transcript in human liver, brain, lung, kidney, and testis; additional transcripts were also observed. The authors stated that the extrahepatic expression of OATP suggests a general role for OATP in transepithelial organic anion transport

Full details of the abovementioned studies are described in the following publications, the disclosure of which are hereby incorporated by reference:
Kullak-Ublick, G. A.; Hagenbuch, B.; Stieger, B.; Schteingart, C. D.; Hofmann, A. F.; Wolkoff, A. W.; Meier, P. J.: Molecular and functional characterization of an organic anion transporting polypeptide cloned from human liver.: Gastroenterology 109: 1274-1282, 1995.; and
By somatic cell hybrid analysis, Kullak-Ublick et al. (1995) mapped the SLC21A3 gene to chromosome 12. Kullak-Ublick et al. (1996) regionally localized the SLC21A3 gene to 12p12 using fluor.

Further studies establishing the function and utilities of SLC21A3 are found in John Hopkins OMIM database record ID 602883, and in sited publications numbered 1213-1214 listed in the bibliography section hereinbelow, which are also hereby incorporated by reference. UDP-glucose Ceramide Glucosyltransferase (UGCG, Accession NM_{—}003358) is another VGAM171 host target gene. UGCG BINDING SITE is HOST TARGET binding site found in the 5′ untranslated region of mRNA encoded by UGCG, corresponding to a HOST TARGET binding site such as BINDING SITE I, BINDING SITE II or BINDING SITE III. Table 2 illustrates the complementarity of the nucleotide sequences of UGCG BINDING SITE, designated SEQ ID:1012, to the nucleotide sequence of VGAM171 RNA, herein designated VGAM RNA, also designated SEQ ID:506.

Another function of VGAM171 is therefore inhibition of UDP-glucose Ceramide Glucosyltransferase (UGCG, Accession NM_{—}003358), a gene which may serve as “flippase” as well as a glucosyltransferase. Accordingly, utilities of VGAM171 include diagnosis, prevention and treatment of diseases and clinical conditions associated with UGCG. The function of UGCG has been established by previous studies. Glycosphingolipids (GSLs) are a group of membrane components that contain lipid and sugar moieties. They are present in essentially all animal cells and are believed to have important roles in various cellular processes. UDP-glucose ceramide glucosyltransferase (UGCG; EC 2.4.1.80) catalyzes the first glycosylation step in GSL biosynthesis. The product, glucosylceramide, is the core structure of more than 300 GSLs. Ichikawa et al. (1996) cloned a UGCG cDNA by expressing a human melanoma cell cDNA library in a UGCG-deficient cell line and screening for UGCG complementation. The predicted 394-amino acid UGCG protein has a potential signal-anchor sequence and a single transmembrane domain near the N terminus, and very hydrophobic regions close to the C terminus, which may interact with the membrane. Northern blot analysis detected a 3.5-kb UGCG transcript in all human tissues examined. By Western blot analysis using antibodies against GCS, or UGCG, Watanabe et al. (1998) detected an approximately 38-kD protein in human keratinocytes. They showed that GCS transcription is up-regulated during keratinocyte differentiation.

Full details of the abovementioned studies are described in the following publications, the disclosure of which are hereby incorporated by reference:
Ichikawa, S.; Sakiyama, H.; Suzuki, G.; Hidari, K. I.-P. J.; Hirabayashi, Y.: Expression cloning of a cDNA for human ceramide glucosyltransferase that catalyzes the first glycosylation step of glycosphingolipid synthesis. Proc. Nat. Acad. Sci. 93: 4638-4643, 1996.; and
Watanabe, R.; Wu, K.; Paul, P.; Marks, D. L.; Kobayashi, T.; Pittelkow, M. R.; Pagano, R. E.: Up-regulation of glucosylceramide synthase expression and activity during human keratinocy.

Further studies establishing the function and utilities of UGCG are found in John Hopkins OMIM database record ID 602874, and in sited publications numbered 1974-1977 listed in the bibliography section hereinbelow, which are also hereby incorporated by reference. MGC3101 (Accession NM_{—}024043) is another VGAM171 host target gene. MGC3101 BINDING SITE is HOST TARGET binding site found in the 3′ untranslated region of mRNA encoded by MGC3101, corresponding to a HOST TARGET binding site such as BINDING SITE I, BINDING SITE II or BINDING SITE III. Table 2 illustrates the complementarity of the nucleotide sequences of MGC3101 BINDING SITE, designated SEQ ID:2047, to the nucleotide sequence of VGAM171 RNA, herein designated VGAM RNA, also designated SEQ ID:506.

Another function of VGAM171 is therefore inhibition of MGC3101 (Accession NM_{—}024043). Accordingly, utilities of VGAM171 include diagnosis, prevention and treatment of diseases and clinical conditions associated with MGC3101. MGC3413 (Accession NM_{—}032678) is another VGAM171 host target gene. MGC3413 BINDING SITE is HOST TARGET binding site found in the 3′ untranslated region of mRNA encoded by MGC3413, corresponding to a HOST TARGET binding site such as BINDING SITE I, BINDING SITE II or BINDING SITE III. Table 2 illustrates the complementarity of the nucleotide sequences of MGC3413 BINDING SITE, designated SEQ ID:2269, to the nucleotide sequence of VGAM171 RNA, herein designated VGAM RNA, also designated SEQ ID:506.

Another function of VGAM171 is therefore inhibition of MGC3413 (Accession NM_{—}032678). Accordingly, utilities of VGAM171 include diagnosis, prevention and treatment of diseases and clinical conditions associated with MGC3413. LOC151568 (Accession NM_{—}138483) is another VGAM171 host target gene. LOC151568 BINDING SITE is HOST TARGET binding site found in the 5′ untranslated region of mRNA encoded by LOC151568, corresponding to a HOST TARGET binding site such as BINDING SITE I, BINDING SITE II or BINDING SITE III. Table 2 illustrates the complementarity of the nucleotide sequences of LOC151568 BINDING SITE, designated SEQ ID:2444, to the nucleotide sequence of VGAM171 RNA, herein designated VGAM RNA, also designated SEQ ID:506.

Another function of VGAM171 is therefore inhibition of LOC151568 (Accession NM_{—}138483). Accordingly, utilities of VGAM171 include diagnosis, prevention and treatment of diseases and clinical conditions associated with LOC151568. LOC152765 (Accession XM_{—}087519) is another VGAM171 host target gene. LOC152765 BINDING SITE is HOST TARGET binding site found in the 5′ untranslated region of mRNA encoded by LOC152765, corresponding to a HOST TARGET binding site such as BINDING SITE I, BINDING SITE II or BINDING SITE III. Table 2 illustrates the complementarity of the nucleotide sequences of LOC152765 BINDING SITE, designated SEQ ID:3175, to the nucleotide sequence of VGAM171 RNA, herein designated VGAM RNA, also designated SEQ ID:506.

Another function of VGAM171 is therefore inhibition of LOC152765 (Accession XM_{—}087519). Accordingly, utilities of VGAM171 include diagnosis, prevention and treatment of diseases and clinical conditions associated with LOC152765. LOC158314 (Accession XM_{—}098920) is another VGAM171 host target gene. LOC158314 BINDING SITE is HOST TARGET binding site found in the 5′ untranslated region of mRNA encoded by LOC158314, corresponding to a HOST TARGET binding site such as BINDING SITE I, BINDING SITE II or BINDING SITE III. Table 2 illustrates the complementarity of the nucleotide sequences of LOC158314 BINDING SITE, designated SEQ ID:3387, to the nucleotide sequence of VGAM171 RNA, herein designated VGAM RNA, also designated SEQ ID:506.

Another function of VGAM171 is therefore inhibition of LOC158314 (Accession XM_{—}098920). Accordingly, utilities of VGAM171 include diagnosis, prevention and treatment of diseases and clinical conditions associated with LOC158314. LOC57086 (Accession NM_{—}020351) is another VGAM171 host target gene. LOC57086 BINDING SITE is HOST TARGET binding site found in the 3′ untranslated region of mRNA encoded by LOC57086, corresponding to a HOST TARGET binding site such as BINDING SITE I, BINDING SITE II or BINDING SITE III. Table 2 illustrates the complementarity of the nucleotide sequences of LOC57086 BINDING SITE, designated SEQ ID:1914, to the nucleotide sequence of VGAM171 RNA, herein designated VGAM RNA, also designated SEQ ID:506.

Another function of VGAM171 is therefore inhibition of LOC57086 (Accession NM_{—}020351). Accordingly, utilities of VGAM171 include diagnosis, prevention and treatment of diseases and clinical conditions associated with LOC57086. FIG. 1 further provides a conceptual description of a novel bioinformatically detected viral gene of the present invention, referred to here as Viral Genomic Address Messenger 172 (VGAM172) viral gene, which modulates expression of respective host target genes thereof, the function and utility of which host target genes is known in the art.

VGAM172 is a novel bioinformatically detected regulatory, non protein coding, viral micro RNA (miRNA) gene. The method by which VGAM172 was detected is described hereinabove with reference to FIGS. 1-8.

VGAM172 gene, herein designated VGAM GENE, is a viral gene contained in the genome of Vaccinia Virus. VGAM172 host target gene, herein designated VGAM HOST TARGET GENE, is a human gene contained in the human genome.

VGAM172 gene encodes a VGAM172 precursor RNA, herein designated VGAM PRECURSOR RNA. Similar to other miRNA genes, and unlike most ordinary genes, VGAM172 precursor RNA does not encode a protein. A nucleotide sequence identical or highly similar to the nucleotide sequence of VGAM172 precursor RNA is designated SEQ ID:158, and is provided hereinbelow with reference to the sequence listing part. Nucleotide sequence SEQ ID:158 is located at position 90682 relative to the genome of Vaccinia Virus.

VGAM172 precursor RNA folds onto itself, forming VGAM172 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM172 folded precursor RNA into VGAM172 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM172 RNA is designated SEQ ID:507, and is provided hereinbelow with reference to the sequence listing part.

VGAM172 host target gene, herein designated VGAM HOST TARGET GENE, encodes a corresponding messenger RNA, VGAM172 host target RNA, herein designated VGAM HOST TARGET RNA. VGAM172 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM172 RNA, herein designated VGAM RNA, binds complementarily to one or more host target binding sites located in untranslated regions of VGAM172 host target RNA, herein designated VGAM HOST TARGET RNA. This complementary binding is due to the fact that the nucleotide sequence of VGAM172 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM172 RNA, herein designated VGAM RNA, may have a different number of host target binding sites in untranslated regions of a VGAM172 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM172 RNA, herein designated VGAM RNA, to host target binding sites on VGAM172 host target RNA, herein designated VGAM HOST TARGET RNA, such as BINDING SITE I, BINDING SITE II and BINDING SITE III, inhibits translation of VGAM172 host target RNA into VGAM172 host target protein, herein designated VGAM HOST TARGET PROTEIN. VGAM host target protein is therefore outlined by a broken line.

It is appreciated that VGAM172 host target gene, herein designated VGAM HOST TARGET GENE, in fact represents a plurality of VGAM172 host target genes. The mRNA of each one of this plurality of VGAM172 host target genes comprises one or more host target binding sites, each having a nucleotide sequence which is at least partly complementary to VGAM172 RNA, herein designated VGAM RNA, and which when bound by VGAM172 RNA causes inhibition of translation of respective one or more VGAM172 host target proteins.

It is further appreciated by one skilled in the art that the mode of translational inhibition illustrated by FIG. 1 with specific reference to translational inhibition exerted by VGAM172 gene, herein designated VGAM GENE, on one or more VGAM172 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM172 is inhibition of expression of host target genes, as part of a novel viral mechanism of attacking a host. Accordingly, utilities of VGAM172 include diagnosis, prevention and treatment of viral infection by Vaccinia Virus. Specific functions, and accordingly utilities, of VGAM172 correlate with, and may be deduced from, the identity of the host target genes which VGAM172 binds and inhibits, and the function of these host target genes, as elaborated hereinbelow.

Nucleotide sequences of the VGAM172 precursor RNA, herein designated VGAM PRECURSOR RNA, and of the ‘diced’ VGAM172 RNA, herein designated VGAM RNA, and a schematic representation of the secondary folding of VGAM172 folded precursor RNA, herein designated VGAM FOLDED PRECURSOR RNA, of VGAM172 are further described hereinbelow with reference to Table 1.

Nucleotide sequences of host target binding sites, such as BINDING SITE-I, BINDING SITE-II and BINDING SITE-III of FIG. 1, found on VGAM172 host target RNA, and schematic representation of the complementarity of each of these host target binding sites to VGAM172 RNA, herein designated VGAM RNA, are described hereinbelow with reference to Table 2.

As mentioned hereinabove with reference to FIG. 1, a function of VGAM172 gene, herein designated VGAM is inhibition of expression of VGAM172 target genes. It is appreciated that specific functions, and accordingly utilities, of VGAM172 correlate with, and may be deduced from, the identity of the target genes which VGAM172 binds and inhibits, and the function of these target genes, as elaborated hereinbelow.

ATP-binding Cassette, Sub-family E (OABP), Member 1 (ABCE1, Accession XM_{—}003555) is a VGAM172 host target gene. ABCE1 BINDING SITE is HOST TARGET binding site found in the 3′ untranslated region of mRNA encoded by ABCE1, corresponding to a HOST TARGET binding site such as BINDING SITE I, BINDING SITE II or BINDING SITE III. Table 2 illustrates the complementarity of the nucleotide sequences of ABCE1 BINDING SITE, designated SEQ ID:2533, to the nucleotide sequence of VGAM172 RNA, herein designated VGAM RNA, also designated SEQ ID: 507.

A function of VGAM172 is therefore inhibition of ATP-binding Cassette, Sub-family E (OABP), Member 1 (ABCE1, Accession XM_{—}003555), a gene which a Ribonuclease L inhibitor. Accordingly, utilities of VGAM172 include diagnosis, prevention and treatment of diseases and clinical conditions associated with ABCE1. The function of ABCE1 has been established by previous studies. The 2-5A/RNase L system is a main pathway for viral interferon (OMIM Ref. No. 147660) action and may play a general role in RNA metabolism. In the pathway, IFN stimulation activates 2-5A synthetases which convert ATP into a set of unusual oligomers known as 2-5A; these oligomers in turn activate RNase L (RNase 4; 180435), which leads to inhibition of protein synthesis by cleaving mRNAs at the 3-prime side of UpNp sequences (Bisbal et al., 1995). Aubry et al. (1996) also cloned the RNS41 gene and found both a 3.8-kb and a 2.4-kb transcript expressed differentially in all tissues examined. Highest expression of the 2.4-kb transcript was found in the testis, while the 3.8-kb transcript was most abundant in ovaries, testis, spleen, and pancreas.

Full details of the abovementioned studies are described in the following publications, the disclosure of which are hereby incorporated by reference:
Bisbal, C.; Martinand, C.; Silhol, M.; Lebleu, B.; Salehzada, T.: Cloning and characterization of a RNase L inhibitor: a new component of the interferon-regulated 2-5A pathway. J. Biol. Chem. 270: 13308-13317, 1995.; and
Aubry, F.; Mattei, M.-G.; Barque, J.-P.; Galibert, F.: Chromosomal localization and expression pattern of the RNase L inhibitor gene. FEBS Lett. 381: 135-139, 1996.

Further studies establishing the function and utilities of ABCE1 are found in John Hopkins OMIM database record ID 601213, and in sited publications numbered 159 and 1594 listed in the bibliography section hereinbelow, which are also hereby incorporated by reference. DMC1 Dosage Suppressor of Mck1 Homolog, Meiosis-specific Homologous Recombination (yeast) (DMC1, Accession NM_{—}007068) is another VGAM172 host target gene. DMC1 BINDING SITE is HOST TARGET binding site found in the 3′ untranslated region of mRNA encoded by DMC1, corresponding to a HOST TARGET binding site such as BINDING SITE I, BINDING SITE II or BINDING SITE III. Table 2 illustrates the complementarity of the nucleotide sequences of DMC1 BINDING SITE, designated SEQ ID:1356, to the nucleotide sequence of VGAM172 RNA, herein designated VGAM RNA, also designated SEQ ID:507.

Another function of VGAM172 is therefore inhibition of DMC1 Dosage Suppressor of Mck1 Homolog, Meiosis-specific Homologous Recombination (yeast) (DMC1, Accession NM_{—}007068). Accordingly, utilities of VGAM172 include diagnosis, prevention and treatment of diseases and clinical conditions associated with DMC1. Pan-hematopoietic Expression (PHEMX, Accession NM_{—}139022) is another VGAM172 host target gene. PHEMX BINDING SITE1 through PHEMX BINDING SITE4 are HOST TARGET binding sites found in untranslated regions of mRNA encoded by PHEMX, corresponding to HOST TARGET binding sites such as BINDING SITE I, BINDING SITE II or BINDING SITE III. Table 2 illustrates the complementarity of the nucleotide sequences of PHEMX BINDING SITE1 through PHEMX BINDING SITE4, designated SEQ ID:2467, SEQ ID:2468, SEQ ID:2469 and SEQ ID: 1237 respectively, to the nucleotide sequence of VGAM172 RNA, herein designated VGAM RNA, also designated SEQ ID:507.

Another function of VGAM172 is therefore inhibition of Pan-hematopoietic Expression (PHEMX, Accession NM_{—}139022), a gene which is an important tumor-suppressor gene region. Accordingly, utilities of VGAM172 include diagnosis, prevention and treatment of diseases and clinical conditions associated with PHEMX. The function of PHEMX has been established by previous studies. Lee et al. (1999) noted that 7 imprinted genes had been identified on 11p15: IGF2 (OMIM Ref. No. 147470), which encodes an important autocrine growth factor in cancer; H19 (OMIM Ref. No. 103280), an untranslated RNA whose imprinting regulates IGF2; ASCL2 (OMIM Ref. No. 601886), a homolog of Drosophila achaete-scute that is expressed in the trophoblast; KCNQ1 (OMIM Ref. No. 192500), which encodes a voltage-gated potassium channel; p57(KIP2) (CDKN1C; 600856), which encodes a cyclin-dependent kinase inhibitor; TSSC5 (IMPT1; 602631), which encodes a predicted transmembrane transporter; and TSSC3 (OMIM Ref. No. 602131), also known as IPL, a homolog of a mouse apoptosis-inducing gene. With the exception of IGF2, all of these genes are expressed from the maternal allele. Because of the large number of imprinted genes on 11p15, spanning approximately 1 Mb, this region appears to represent 1 of 2 known large imprinted domains in the human genome, the other being the Prader-Willi/Angelman syndrome domain of 15q11-q13 (see OMIM Ref. No. 105830). Koi et al. (1993) isolated a sub-chromosomal transferable fragment (STF) that suppresses in vitro growth of the rhabdomyosarcoma cell line RD, confirming the existence of 1 or more tumor suppressor genes within this region. Hu et al. (1997) found that the STF spans approximately 2.5 Mb, with D11S12 at its proximal end and D11S1318 at its distal end. Within a cluster of imprinted genes in this STF, Lee et al. (1999) identified 2 novel genes, designated TSSC4 (OMIM Ref. No. 603852) and TSSC6, that were not imprinted in any of the fetal or extraembryonic tissues examined. The TSSC6 cDNA encodes a predicted protein of 290 amino acids that shows no close similarity to previously reported proteins. Northern blot analysis failed to detect TSSC6 gene expression; however, RT-PCR analysis suggested that the TSSC6 gene is ubiquitously expressed at low levels. The TSSC4 and TSSC6 genes are both located in the center of the 1-Mb imprinted domain on 11p15 that contains the 7 imprinted genes. Thus, the imprinted gene domain of 1p15 appears to contain at least 2 imprinted subdomains, between which the TSSC4 and TSSC6 genes substantially escape imprinting, due either to a lack of initial silencing or to an early developmental relaxation of imprinting

Full details of the abovementioned studies are described in the following publications, the disclosure of which are hereby incorporated by reference:
Koi, M.; Johnson, L. A.; Kalikin, L. M.; Little, P. F. R.; Nakamura, Y.; Feinberg, A. P.: Tumor cell growth arrest caused by subchromosomal transferable DNA fragments from chromosome 11. Science 260: 361-364, 1993.; and
Lee, M. P.; Brandenburg, S.; Landes, G. M.; Adams, M.; Miller, G.; Feinberg, A. P.: Two novel genes in the center of the 11p15 imprinted domain escape genomic imprinting. Hum. Molec. Ge.

Further studies establishing the function and utilities of PHEMX are found in John Hopkins OMIM database record ID 603853, and in sited publications numbered 111 and 1671 listed in the bibliography section hereinbelow, which are also hereby incorporated by reference. KIAA0979 (Accession NM_{—}015032) is another VGAM172 host target gene. KIAA0979 BINDING SITE is HOST TARGET binding site found in the 3′ untranslated region of mRNA encoded by KIAA0979, corresponding to a HOST TARGET binding site such as BINDING SITE I, BINDING SITE II or BINDING SITE III. Table 2 illustrates the complementarity of the nucleotide sequences of KIAA0979 BINDING SITE, designated SEQ ID: 1602, to the nucleotide sequence of VGAM172 RNA, herein designated VGAM RNA, also designated SEQ ID: 507.

Another function of VGAM172 is therefore inhibition of KIAA0979 (Accession NM_{—}015032). Accordingly, utilities of VGAM172 include diagnosis, prevention and treatment of diseases and clinical conditions associated with KIAA0979. LOC220672 (Accession XM_{—}017177) is another VGAM172 host target gene. LOC220672 BINDING SITE is HOST TARGET binding site found in the 3′ untranslated region of mRNA encoded by LOC220672, corresponding to a HOST TARGET binding site such as BINDING SITE I, BINDING SITE II or BINDING SITE III. Table 2 illustrates the complementarity of the nucleotide sequences of LOC220672 BINDING SITE, designated SEQ ID:2566, to the nucleotide sequence of VGAM172 RNA, herein designated VGAM RNA, also designated SEQ ID:507.

Another function of VGAM172 is therefore inhibition of LOC220672 (Accession XM_{—}017177). Accordingly, utilities of VGAM172 include diagnosis, prevention and treatment of diseases and clinical conditions associated with LOC220672. LOC257336 (Accession XM_{—}171216) is another VGAM172 host target gene. LOC257336 BINDING SITE is HOST TARGET binding site found in the 5′ untranslated region of mRNA encoded by LOC257336, corresponding to a HOST TARGET binding site such as BINDING SITE I, BINDING SITE II or BINDING SITE III. Table 2 illustrates the complementarity of the nucleotide sequences of LOC257336 BINDING SITE, designated SEQ ID:3694, to the nucleotide sequence of VGAM172 RNA, herein designated VGAM RNA, also designated SEQ ID:507.

Another function of VGAM172 is therefore inhibition of LOC257336 (Accession XM_{—}171216). Accordingly, utilities of VGAM172 include diagnosis, prevention and treatment of diseases and clinical conditions associated with LOC257336. FIG. 1 further provides a conceptual description of a novel bioinformatically detected viral gene of the present invention, referred to here as Viral Genomic Address Messenger 173 (VGAM173) viral gene, which modulates expression of respective host target genes thereof, the function and utility of which host target genes is known in the art.

VGAM173 is a novel bioinformatically detected regulatory, non protein coding, viral micro RNA (miRNA) gene. The method by which VGAM173 was detected is described hereinabove with reference to FIGS. 1-8.

VGAM173 gene, herein designated VGAM GENE, is a viral gene contained in the genome of Vaccinia Virus. VGAM173 host target gene, herein designated VGAM HOST TARGET GENE, is a human gene contained in the human genome.

VGAM173 gene encodes a VGAM173 precursor RNA, herein designated VGAM PRECURSOR RNA. Similar to other miRNA genes, and unlike most ordinary genes, VGAM173 precursor RNA does not encode a protein. A nucleotide sequence identical or highly similar to the nucleotide sequence of VGAM173 precursor RNA is designated SEQ ID:159, and is provided hereinbelow with reference to the sequence listing part. Nucleotide sequence SEQ ID:159 is located at position 90131 relative to the genome of Vaccinia Virus.

VGAM173 precursor RNA folds onto itself, forming VGAM173 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM173 folded precursor RNA into VGAM173 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM173 RNA is designated SEQ ID:508, and is provided hereinbelow with reference to the sequence listing part.

VGAM173 host target gene, herein designated VGAM HOST TARGET GENE, encodes a corresponding messenger RNA, VGAM173 host target RNA, herein designated VGAM HOST TARGET RNA. VGAM173 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM173 RNA, herein designated VGAM RNA, binds complementarily to one or more host target binding sites located in untranslated regions of VGAM173 host target RNA, herein designated VGAM HOST TARGET RNA. This complementary binding is due to the fact that the nucleotide sequence of VGAM173 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM173 RNA, herein designated VGAM RNA, may have a different number of host target binding sites in untranslated regions of a VGAM173 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM173 RNA, herein designated VGAM RNA, to host target binding sites on VGAM173 host target RNA, herein designated VGAM HOST TARGET RNA, such as BINDING SITE I, BINDING SITE II and BINDING SITE III, inhibits translation of VGAM173 host target RNA into VGAM173 host target protein, herein designated VGAM HOST TARGET PROTEIN. VGAM host target protein is therefore outlined by a broken line.

It is appreciated that VGAM173 host target gene, herein designated VGAM HOST TARGET GENE, in fact represents a plurality of VGAM173 host target genes. The mRNA of each one of this plurality of VGAM173 host target genes comprises one or more host target binding sites, each having a nucleotide sequence which is at least partly complementary to VGAM173 RNA, herein designated VGAM RNA, and which when bound by VGAM173 RNA causes inhibition of translation of respective one or more VGAM173 host target proteins.

It is further appreciated by one skilled in the art that the mode of translational inhibition illustrated by FIG. 1 with specific reference to translational inhibition exerted by VGAM173 gene, herein designated VGAM GENE, on one or more VGAM173 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM173 is inhibition of expression of host target genes, as part of a novel viral mechanism of attacking a host. Accordingly, utilities of VGAM173 include diagnosis, prevention and treatment of viral infection by Vaccinia Virus. Specific functions, and accordingly utilities, of VGAM173 correlate with, and may be deduced from, the identity of the host target genes which VGAM173 binds and inhibits, and the function of these host target genes, as elaborated hereinbelow.

Nucleotide sequences of the VGAM173 precursor RNA, herein designated VGAM PRECURSOR RNA, and of the ‘diced’ VGAM173 RNA, herein designated VGAM RNA, and a schematic representation of the secondary folding of VGAM173 folded precursor RNA, herein designated VGAM FOLDED PRECURSOR RNA, of VGAM173 are further described hereinbelow with reference to Table 1.

Nucleotide sequences of host target binding sites, such as BINDING SITE-I, BINDING SITE-II and BINDING SITE-III of FIG. 1, found on VGAM173 host target RNA, and schematic representation of the complementarity of each of these host target binding sites to VGAM173 RNA, herein designated VGAM RNA, are described hereinbelow with reference to Table 2.

As mentioned hereinabove with reference to FIG. 1, a function of VGAM173 gene, herein designated VGAM is inhibition of expression of VGAM173 target genes. It is appreciated that specific functions, and accordingly utilities, of VGAM173 correlate with, and may be deduced from, the identity of the target genes which VGAM173 binds and inhibits, and the function of these target genes, as elaborated hereinbelow.

MGC16169 (Accession NM_{—}033115) is a VGAM173 host target gene. MGC16169 BINDING SITE is HOST TARGET binding site found in the 3′ untranslated region of mRNA encoded by MGC16169, corresponding to a HOST TARGET binding site such as BINDING SITE I, BINDING SITE II or BINDING SITE III. Table 2 illustrates the complementarity of the nucleotide sequences of MGC16169 BINDING SITE, designated SEQ ID:2312, to the nucleotide sequence of VGAM173 RNA, herein designated VGAM RNA, also designated SEQ ID:508.

A function of VGAM173 is therefore inhibition of MGC16169 (Accession NM_{—}033115). Accordingly, utilities of VGAM173 include diagnosis, prevention and treatment of diseases and clinical conditions associated with MGC16169. LOC151414 (Accession XM_{—}087197) is another VGAM173 host target gene. LOC151414 BINDING SITE is HOST TARGET binding site found in the 3′ untranslated region of mRNA encoded by LOC151414, corresponding to a HOST TARGET binding site such as BINDING SITE I, BINDING SITE II or BINDING SITE III. Table 2 illustrates the complementarity of the nucleotide sequences of LOC151414 BINDING SITE, designated SEQ ID:3156, to the nucleotide sequence of VGAM173 RNA, herein designated VGAM RNA, also designated SEQ ID:508.

Another function of VGAM173 is therefore inhibition of LOC151414 (Accession XM_{—}087197). Accordingly, utilities of VGAM173 include diagnosis, prevention and treatment of diseases and clinical conditions associated with LOC151414. LOC153817 (Accession XM_{—}027964) is another VGAM173 host target gene. LOC153817 BINDING SITE is HOST TARGET binding site found in the 5′ untranslated region of mRNA encoded by LOC153817, corresponding to a HOST TARGET binding site such as BINDING SITE I, BINDING SITE II or BINDING SITE III. Table 2 illustrates the complementarity of the nucleotide sequences of LOC153817 BINDING SITE, designated SEQ ID:2591, to the nucleotide sequence of VGAM173 RNA, herein designated VGAM RNA, also designated SEQ ID:508.

Another function of VGAM173 is therefore inhibition of LOC153817 (Accession XM_{—}027964). Accordingly, utilities of VGAM173 include diagnosis, prevention and treatment of diseases and clinical conditions associated with LOC153817. LOC51145 (Accession NM_{—}016158) is another VGAM173 host target gene. LOC51145 BINDING SITE is HOST TARGET binding site found in the 3′ untranslated region of mRNA encoded by LOC51145, corresponding to a HOST TARGET binding site such as BINDING SITE I, BINDING SITE II or BINDING SITE III. Table 2 illustrates the complementarity of the nucleotide sequences of LOC51145 BINDING SITE, designated SEQ ID:1665, to the nucleotide sequence of VGAM173 RNA, herein designated VGAM RNA, also designated SEQ ID:508.

Another function of VGAM173 is therefore inhibition of LOC51145 (Accession NM_{—}016158). Accordingly, utilities of VGAM173 include diagnosis, prevention and treatment of diseases and clinical conditions associated with LOC51145. FIG. 1 further provides a conceptual description of a novel bioinformatically detected viral gene of the present invention, referred to here as Viral Genomic Address Messenger 174 (VGAM174) viral gene, which modulates expression of respective host target genes thereof, the function and utility of which host target genes is known in the art.

VGAM174 is a novel bioinformatically detected regulatory, non protein coding, viral micro RNA (miRNA) gene. The method by which VGAM174 was detected is described hereinabove with reference to FIGS. 1-8.

VGAM174 gene, herein designated VGAM GENE, is a viral gene contained in the genome of Vaccinia Virus. VGAM174 host target gene, herein designated VGAM HOST TARGET GENE, is a human gene contained in the human genome.

VGAM174 gene encodes a VGAM174 precursor RNA, herein designated VGAM PRECURSOR RNA. Similar to other miRNA genes, and unlike most ordinary genes, VGAM174 precursor RNA does not encode a protein. A nucleotide sequence identical or highly similar to the nucleotide sequence of VGAM174 precursor RNA is designated SEQ ID:160, and is provided hereinbelow with reference to the sequence listing part. Nucleotide sequence SEQ ID: 160 is located at position 89784 relative to the genome of Vaccinia Virus.

VGAM174 precursor RNA folds onto itself, forming VGAM174 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM174 folded precursor RNA into VGAM174 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM174 RNA is designated SEQ ID:509, and is provided hereinbelow with reference to the sequence listing part.

VGAM174 host target gene, herein designated VGAM HOST TARGET GENE, encodes a corresponding messenger RNA, VGAM174 host target RNA, herein designated VGAM HOST TARGET RNA. VGAM174 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM174 RNA, herein designated VGAM RNA, binds complementarily to one or more host target binding sites located in untranslated regions of VGAM174 host target RNA, herein designated VGAM HOST TARGET RNA. This complementary binding is due to the fact that the nucleotide sequence of VGAM174 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM174 RNA, herein designated VGAM RNA, may have a different number of host target binding sites in untranslated regions of a VGAM174 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM174 RNA, herein designated VGAM RNA, to host target binding sites on VGAM174 host target RNA, herein designated VGAM HOST TARGET RNA, such as BINDING SITE I, BINDING SITE II and BINDING SITE III, inhibits translation of VGAM174 host target RNA into VGAM174 host target protein, herein designated VGAM HOST TARGET PROTEIN. VGAM host target protein is therefore outlined by a broken line.

It is appreciated that VGAM174 host target gene, herein designated VGAM HOST TARGET GENE, in fact represents a plurality of VGAM174 host target genes. The mRNA of each one of this plurality of VGAM174 host target genes comprises one or more host target binding sites, each having a nucleotide sequence which is at least partly complementary to VGAM174 RNA, herein designated VGAM RNA, and which when bound by VGAM174 RNA causes inhibition of translation of respective one or more VGAM174 host target proteins.

It is further appreciated by one skilled in the art that the mode of translational inhibition illustrated by FIG. 1 with specific reference to translational inhibition exerted by VGAM174 gene, herein designated VGAM GENE, on one or more VGAM174 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM174 is inhibition of expression of host target genes, as part of a novel viral mechanism of attacking a host. Accordingly, utilities of VGAM174 include diagnosis, prevention and treatment of viral infection by Vaccinia Virus. Specific functions, and accordingly utilities, of VGAM174 correlate with, and may be deduced from, the identity of the host target genes which VGAM174 binds and inhibits, and the function of these host target genes, as elaborated hereinbelow.

Nucleotide sequences of the VGAM174 precursor RNA, herein designated VGAM PRECURSOR RNA, and of the ‘diced’ VGAM174 RNA, herein designated VGAM RNA, and a schematic representation of the secondary folding of VGAM174 folded precursor RNA, herein designated VGAM FOLDED PRECURSOR RNA, of VGAM174 are further described hereinbelow with reference to Table 1.

Nucleotide sequences of host target binding sites, such as BINDING SITE-I, BINDING SITE-II and BINDING SITE-III of FIG. 1, found on VGAM174 host target RNA, and schematic representation of the complementarity of each of these host target binding sites to VGAM174 RNA, herein designated VGAM RNA, are described hereinbelow with reference to Table 2.

As mentioned hereinabove with reference to FIG. 1, a function of VGAM174 gene, herein designated VGAM is inhibition of expression of VGAM174 target genes. It is appreciated that specific functions, and accordingly utilities, of VGAM174 correlate with, and may be deduced from, the identity of the target genes which VGAM174 binds and inhibits, and the function of these target genes, as elaborated hereinbelow.

Enamelin (ENAM, Accession NM_{—}031889) is a VGAM174 host target gene. ENAM BINDING SITE is HOST TARGET binding site found in the 5′ untranslated region of mRNA encoded by ENAM, corresponding to a HOST TARGET binding site such as BINDING SITE I, BINDING SITE II or BINDING SITE III. Table 2 illustrates the complementarity of the nucleotide sequences of ENAM BINDING SITE, designated SEQ ID:2215, to the nucleotide sequence of VGAM174 RNA, herein designated VGAM RNA, also designated SEQ ID:509.

A function of VGAM174 is therefore inhibition of Enamelin (ENAM, Accession NM_{—}031889). Accordingly, utilities of VGAM174 include diagnosis, prevention and treatment of diseases and clinical conditions associated with ENAM. Dedicator of Cyto-kinesis 3 (DOCK3, Accession XM_{—}039259) is another VGAM174 host target gene. DOCK3 BINDING SITE is HOST TARGET binding site found in the 3′ untranslated region of mRNA encoded by DOCK3, corresponding to a HOST TARGET binding site such as BINDING SITE I, BINDING SITE II or BINDING SITE III. Table 2 illustrates the complementarity of the nucleotide sequences of DOCK3 BINDING SITE, designated SEQ ID:2750, to the nucleotide sequence of VGAM174 RNA, herein designated VGAM RNA, also designated SEQ ID:509.

Another function of VGAM174 is therefore inhibition of Dedicator of Cyto-kinesis 3 (DOCK3, Accession XM_{—}039259). Accordingly, utilities of VGAM174 include diagnosis, prevention and treatment of diseases and clinical conditions associated with DOCK3. GAPCENA (Accession NM_{—}012197) is another VGAM174 host target gene. GAPCENA BINDING SITE is HOST TARGET binding site found in the 3′ untranslated region of mRNA encoded by GAPCENA, corresponding to a HOST TARGET binding site such as BINDING SITE I, BINDING SITE II or BINDING SITE III. Table 2 illustrates the complementarity of the nucleotide sequences of GAPCENA BINDING SITE, designated SEQ ID: 1412, to the nucleotide sequence of VGAM174 RNA, herein designated VGAM RNA, also designated SEQ ID:509.

Another function of VGAM174 is therefore inhibition of GAPCENA (Accession NM_{—}012197). Accordingly, utilities of VGAM174 include diagnosis, prevention and treatment of diseases and clinical conditions associated with GAPCENA. FIG. 1 further provides a conceptual description of a novel bioinformatically detected viral gene of the present invention, referred to here as Viral Genomic Address Messenger 175 (VGAM175) viral gene, which modulates expression of respective host target genes thereof, the function and utility of which host target genes is known in the art.

VGAM175 is a novel bioinformatically detected regulatory, non protein coding, viral micro RNA (miRNA) gene. The method by which VGAM175 was detected is described hereinabove with reference to FIGS. 1-8.

VGAM175 gene, herein designated VGAM GENE, is a viral gene contained in the genome of Vaccinia Virus. VGAM175 host target gene, herein designated VGAM HOST TARGET GENE, is a human gene contained in the human genome.

VGAM175 gene encodes a VGAM175 precursor RNA, herein designated VGAM PRECURSOR RNA. Similar to other miRNA genes, and unlike most ordinary genes, VGAM175 precursor RNA does not encode a protein. A nucleotide sequence identical or highly similar to the nucleotide sequence of VGAM175 precursor RNA is designated SEQ ID:161, and is provided hereinbelow with reference to the sequence listing part. Nucleotide sequence SEQ ID: 161 is located at position 91213 relative to the genome of Vaccinia Virus.

VGAM175 precursor RNA folds onto itself, forming VGAM175 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM175 folded precursor RNA into VGAM175 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM175 RNA is designated SEQ ID:510, and is provided hereinbelow with reference to the sequence listing part.

VGAM175 host target gene, herein designated VGAM HOST TARGET GENE, encodes a corresponding messenger RNA, VGAM175 host target RNA, herein designated VGAM HOST TARGET RNA. VGAM175 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM175 RNA, herein designated VGAM RNA, binds complementarily to one or more host target binding sites located in untranslated regions of VGAM175 host target RNA, herein designated VGAM HOST TARGET RNA. This complementary binding is due to the fact that the nucleotide sequence of VGAM175 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM175 RNA, herein designated VGAM RNA, may have a different number of host target binding sites in untranslated regions of a VGAM175 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM175 RNA, herein designated VGAM RNA, to host target binding sites on VGAM175 host target RNA, herein designated VGAM HOST TARGET RNA, such as BINDING SITE I, BINDING SITE II and BINDING SITE III, inhibits translation of VGAM175 host target RNA into VGAM175 host target protein, herein designated VGAM HOST TARGET PROTEIN. VGAM host target protein is therefore outlined by a broken line.

It is appreciated that VGAM175 host target gene, herein designated VGAM HOST TARGET GENE, in fact represents a plurality of VGAM175 host target genes. The mRNA of each one of this plurality of VGAM175 host target genes comprises one or more host target binding sites, each having a nucleotide sequence which is at least partly complementary to VGAM175 RNA, herein designated VGAM RNA, and which when bound by VGAM175 RNA causes inhibition of translation of respective one or more VGAM175 host target proteins.

It is further appreciated by one skilled in the art that the mode of translational inhibition illustrated by FIG. 1 with specific reference to translational inhibition exerted by VGAM175 gene, herein designated VGAM GENE, on one or more VGAM175 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM175 is inhibition of expression of host target genes, as part of a novel viral mechanism of attacking a host. Accordingly, utilities of VGAM175 include diagnosis, prevention and treatment of viral infection by Vaccinia Virus. Specific functions, and accordingly utilities, of VGAM175 correlate with, and may be deduced from, the identity of the host target genes which VGAM175 binds and inhibits, and the function of these host target genes, as elaborated hereinbelow.

Nucleotide sequences of the VGAM175 precursor RNA, herein designated VGAM PRECURSOR RNA, and of the ‘diced’ VGAM175 RNA, herein designated VGAM RNA, and a schematic representation of the secondary folding of VGAM175 folded precursor RNA, herein designated VGAM FOLDED PRECURSOR RNA, of VGAM175 are further described hereinbelow with reference to Table 1.

Nucleotide sequences of host target binding sites, such as BINDING SITE-I, BINDING SITE-II and BINDING SITE-III of FIG. 1, found on VGAM175 host target RNA, and schematic representation of the complementarity of each of these host target binding sites to VGAM175 RNA, herein designated VGAM RNA, are described hereinbelow with reference to Table 2.

As mentioned hereinabove with reference to FIG. 1, a function of VGAM175 gene, herein designated VGAM is inhibition of expression of VGAM175 target genes. It is appreciated that specific functions, and accordingly utilities, of VGAM175 correlate with, and may be deduced from, the identity of the target genes which VGAM175 binds and inhibits, and the function of these target genes, as elaborated hereinbelow.

Heterogeneous Nuclear Ribonucleoprotein A2/B1 (HNRPA2B1, Accession NM_{—}002137) is a VGAM175 host target gene. HNRPA2B1 BINDING SITE1 and HNRPA2B1 BINDING SITE2 are HOST TARGET binding sites found in untranslated regions of mRNA encoded by HNRPA2B1, corresponding to HOST TARGET binding sites such as BINDING SITE I, BINDING SITE II or BINDING SITE III. Table 2 illustrates the complementarity of the nucleotide sequences of HNRPA2B1 BINDING SITE1 and HNRPA2B1 BINDING SITE2, designated SEQ ID:902 and SEQ ID:2186 respectively, to the nucleotide sequence of VGAM175 RNA, herein designated VGAM RNA, also designated SEQ ID: 510.

A function of VGAM175 is therefore inhibition of Heterogeneous Nuclear Ribonucleoprotein A2/B1 (HNRPA2B1, Accession NM_{—}002137), a gene which involves with pre-mrna processing. Accordingly, utilities of VGAM175 include diagnosis, prevention and treatment of diseases and clinical conditions associated with HNRPA2B1. The function of HNRPA2B1 has been established by previous studies. The splicing of pre-mRNA into the mature product occurs in a multicomponent complex constituted by small nuclear ribonucleoprotein particles (snRNP) and proteins such as splicing factors and heterogeneous nuclear ribonucleoprotein (hnRNP) proteins. The hnRNP proteins are a family of at least 20 polypeptides whose precise role in RNA processing remains to be fully defined. The hnRNP A2 belongs, with A1, B1, and B2, to the basic protein subset of the hnRNP complex. All these proteins share a modular structure consisting of 2 conserved RNA binding domains linked to less conserved gly-rich domains. Biamonti et al. (1994) isolated and sequenced the A2 gene and compared it with the previously described A1 gene (HNRPA1; 164017). The HNRPA2 gene was mapped to 7p15 by fluorescence in situ hybridization. It was found to be split into 12 exons, including an alternatively spliced 36 nucleotide mini-exon specific for the human hnRNP protein B1. Biamonti et al. (1994) showed that the intron/exon organization of the A2 gene is identical to that of the A1 gene over its entire length, indicating a common origin by gene duplication. Kozu et al. (1995) also cloned the HNRPA2B1 gene and showed that it is 9-kb long and produces both the B1 and A2/B1 mRNAs as a consequence of alternative splicing. The transcripts were found in all cells with the B1 isoform occurring at about 2-5% of the A2/B1 level. See nuclear ribonucleoprotein particle Cl protein (OMIM Ref. No. 164020).

Full details of the abovementioned studies are described in the following publications, the disclosure of which are hereby incorporated by reference:
Biamonti, G.; Ruggiu, M.; Saccone, S.; Della Valle, G.; Riva, S.: Two homologous genes, originated by duplication, encode the human hnRNP proteins A2 and A1. Nucleic Acids Res. 22: 1996-2002, 1994.; and
Kozu, T.; Henrich, B.; Schafer, K. P.: Structure and expression of the gene (HNRPA2B1) encoding the human hnRNP protein A2/B1. Genomics 25: 365-371, 1995.

Further studies establishing the function and utilities of HNRPA2B1 are found in John Hopkins OMIM database record ID 600124, and in sited publications numbered 2014 listed in the bibliography section hereinbelow, which are also hereby incorporated by reference. LOC146481 (Accession XM_{—}085484) is another VGAM175 host target gene. LOC146481 BINDING SITE is HOST TARGET binding site found in the 3′ untranslated region of mRNA encoded by LOC146481, corresponding to a HOST TARGET binding site such as BINDING SITE I, BINDING SITE II or BINDING SITE III. Table 2 illustrates the complementarity of the nucleotide sequences of LOC146481 BINDING SITE, designated SEQ ID:3092, to the nucleotide sequence of VGAM175 RNA, herein designated VGAM RNA, also designated SEQ ID:510.

Another function of VGAM175 is therefore inhibition of LOC146481 (Accession XM_{—}085484). Accordingly, utilities of VGAM175 include diagnosis, prevention and treatment of diseases and clinical conditions associated with LOC146481. FIG. 1 further provides a conceptual description of a novel bioinformatically detected viral gene of the present invention, referred to here as Viral Genomic Address Messenger 176 (VGAM176) viral gene, which modulates expression of respective host target genes thereof, the function and utility of which host target genes is known in the art.

VGAM176 is a novel bioinformatically detected regulatory, non protein coding, viral micro RNA (miRNA) gene. The method by which VGAM176 was detected is described hereinabove with reference to FIGS. 1-8.

VGAM176 gene, herein designated VGAM GENE, is a viral gene contained in the genome of Vaccinia Virus. VGAM176 host target gene, herein designated VGAM HOST TARGET GENE, is a human gene contained in the human genome.

VGAM176 gene encodes a VGAM176 precursor RNA, herein designated VGAM PRECURSOR RNA. Similar to other miRNA genes, and unlike most ordinary genes, VGAM176 precursor RNA does not encode a protein. A nucleotide sequence identical or highly similar to the nucleotide sequence of VGAM176 precursor RNA is designated SEQ ID:162, and is provided hereinbelow with reference to the sequence listing part. Nucleotide sequence SEQ ID: 162 is located at position 90963 relative to the genome of Vaccinia Virus.

VGAM176 precursor RNA folds onto itself, forming VGAM176 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM176 folded precursor RNA into VGAM176 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM176 RNA is designated SEQ ID:511, and is provided hereinbelow with reference to the sequence listing part.

VGAM176 host target gene, herein designated VGAM HOST TARGET GENE, encodes a corresponding messenger RNA, VGAM176 host target RNA, herein designated VGAM HOST TARGET RNA. VGAM176 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM176 RNA, herein designated VGAM RNA, binds complementarily to one or more host target binding sites located in untranslated regions of VGAM176 host target RNA, herein designated VGAM HOST TARGET RNA. This complementary binding is due to the fact that the nucleotide sequence of VGAM176 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM176 RNA, herein designated VGAM RNA, may have a different number of host target binding sites in untranslated regions of a VGAM176 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM176 RNA, herein designated VGAM RNA, to host target binding sites on VGAM176 host target RNA, herein designated VGAM HOST TARGET RNA, such as BINDING SITE I, BINDING SITE II and BINDING SITE III, inhibits translation of VGAM176 host target RNA into VGAM176 host target protein, herein designated VGAM HOST TARGET PROTEIN. VGAM host target protein is therefore outlined by a broken line.

It is appreciated that VGAM176 host target gene, herein designated VGAM HOST TARGET GENE, in fact represents a plurality of VGAM176 host target genes. The mRNA of each one of this plurality of VGAM176 host target genes comprises one or more host target binding sites, each having a nucleotide sequence which is at least partly complementary to VGAM176 RNA, herein designated VGAM RNA, and which when bound by VGAM176 RNA causes inhibition of translation of respective one or more VGAM176 host target proteins.

It is further appreciated by one skilled in the art that the mode of translational inhibition illustrated by FIG. 1 with specific reference to translational inhibition exerted by VGAM176 gene, herein designated VGAM GENE, on one or more VGAM176 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM176 is inhibition of expression of host target genes, as part of a novel viral mechanism of attacking a host. Accordingly, utilities of VGAM176 include diagnosis, prevention and treatment of viral infection by Vaccinia Virus. Specific functions, and accordingly utilities, of VGAM176 correlate with, and may be deduced from, the identity of the host target genes which VGAM176 binds and inhibits, and the function of these host target genes, as elaborated hereinbelow.

Nucleotide sequences of the VGAM176 precursor RNA, herein designated VGAM PRECURSOR RNA, and of the ‘diced’ VGAM176 RNA, herein designated VGAM RNA, and a schematic representation of the secondary folding of VGAM176 folded precursor RNA, herein designated VGAM FOLDED PRECURSOR RNA, of VGAM176 are further described hereinbelow with reference to Table 1.

Nucleotide sequences of host target binding sites, such as BINDING SITE-I, BINDING SITE-II and BINDING SITE-III of FIG. 1, found on VGAM176 host target RNA, and schematic representation of the complementarity of each of these host target binding sites to VGAM176 RNA, herein designated VGAM RNA, are described hereinbelow with reference to Table 2.

As mentioned hereinabove with reference to FIG. 1, a function of VGAM176 gene, herein designated VGAM is inhibition of expression of VGAM176 target genes. It is appreciated that specific functions, and accordingly utilities, of VGAM176 correlate with, and may be deduced from, the identity of the target genes which VGAM176 binds and inhibits, and the function of these target genes, as elaborated hereinbelow.

LOC146229 (Accession XM_{—}085387) is a VGAM176 host target gene. LOC146229 BINDING SITE is HOST TARGET binding site found in the 3′ untranslated region of mRNA encoded by LOC146229, corresponding to a HOST TARGET binding site such as BINDING SITE I, BINDING SITE II or BINDING SITE III. Table 2 illustrates the complementarity of the nucleotide sequences of LOC146229 BINDING SITE, designated SEQ ID:3087, to the nucleotide sequence of VGAM176 RNA, herein designated VGAM RNA, also designated SEQ ID:511.

A function of VGAM176 is therefore inhibition of LOC146229 (Accession XM_{—}085387). Accordingly, utilities of VGAM176 include diagnosis, prevention and treatment of diseases and clinical conditions associated with LOC146229. FIG. 1 further provides a conceptual description of a novel bioinformatically detected viral gene of the present invention, referred to here as Viral Genomic Address Messenger 177 (VGAM177) viral gene, which modulates expression of respective host target genes thereof, the function and utility of which host target genes is known in the art.

VGAM177 is a novel bioinformatically detected regulatory, non protein coding, viral micro RNA (miRNA) gene. The method by which VGAM177 was detected is described hereinabove with reference to FIGS. 1-8.

VGAM177 gene, herein designated VGAM GENE, is a viral gene contained in the genome of Vaccinia Virus. VGAM177 host target gene, herein designated VGAM HOST TARGET GENE, is a human gene contained in the human genome.

VGAM177 gene encodes a VGAM177 precursor RNA, herein designated VGAM PRECURSOR RNA. Similar to other miRNA genes, and unlike most ordinary genes, VGAM177 precursor RNA does not encode a protein. A nucleotide sequence identical or highly similar to the nucleotide sequence of VGAM177 precursor RNA is designated SEQ ID:163, and is provided hereinbelow with reference to the sequence listing part. Nucleotide sequence SEQ ID: 163 is located at position 92476 relative to the genome of Vaccinia Virus.

VGAM177 precursor RNA folds onto itself, forming VGAM177 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM177 folded precursor RNA into VGAM177 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM177 RNA is designated SEQ ID:512, and is provided hereinbelow with reference to the sequence listing part.

VGAM177 host target gene, herein designated VGAM HOST TARGET GENE, encodes a corresponding messenger RNA, VGAM177 host target RNA, herein designated VGAM HOST TARGET RNA. VGAM177 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM177 RNA, herein designated VGAM RNA, binds complementarily to one or more host target binding sites located in untranslated regions of VGAM177 host target RNA, herein designated VGAM HOST TARGET RNA. This complementary binding is due to the fact that the nucleotide sequence of VGAM177 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM177 RNA, herein designated VGAM RNA, may have a different number of host target binding sites in untranslated regions of a VGAM177 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM177 RNA, herein designated VGAM RNA, to host target binding sites on VGAM177 host target RNA, herein designated VGAM HOST TARGET RNA, such as BINDING SITE I, BINDING SITE II and BINDING SITE III, inhibits translation of VGAM177 host target RNA into VGAM177 host target protein, herein designated VGAM HOST TARGET PROTEIN. VGAM host target protein is therefore outlined by a broken line.

It is appreciated that VGAM177 host target gene, herein designated VGAM HOST TARGET GENE, in fact represents a plurality of VGAM177 host target genes. The mRNA of each one of this plurality of VGAM177 host target genes comprises one or more host target binding sites, each having a nucleotide sequence which is at least partly complementary to VGAM177 RNA, herein designated VGAM RNA, and which when bound by VGAM177 RNA causes inhibition of translation of respective one or more VGAM177 host target proteins.

It is further appreciated by one skilled in the art that the mode of translational inhibition illustrated by FIG. 1 with specific reference to translational inhibition exerted by VGAM177 gene, herein designated VGAM GENE, on one or more VGAM177 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM177 is inhibition of expression of host target genes, as part of a novel viral mechanism of attacking a host. Accordingly, utilities of VGAM177 include diagnosis, prevention and treatment of viral infection by Vaccinia Virus. Specific functions, and accordingly utilities, of VGAM177 correlate with, and may be deduced from, the identity of the host target genes which VGAM177 binds and inhibits, and the function of these host target genes, as elaborated hereinbelow.

Nucleotide sequences of the VGAM177 precursor RNA, herein designated VGAM PRECURSOR RNA, and of the ‘diced’ VGAM177 RNA, herein designated VGAM RNA, and a schematic representation of the secondary folding of VGAM177 folded precursor RNA, herein designated VGAM FOLDED PRECURSOR RNA, of VGAM177 are further described hereinbelow with reference to Table 1.

Nucleotide sequences of host target binding sites, such as BINDING SITE-I, BINDING SITE-II and BINDING SITE-III of FIG. 1, found on VGAM177 host target RNA, and schematic representation of the complementarity of each of these host target binding sites to VGAM177 RNA, herein designated VGAM RNA, are described hereinbelow with reference to Table 2.

As mentioned hereinabove with reference to FIG. 1, a function of VGAM177 gene, herein designated VGAM is inhibition of expression of VGAM177 target genes. It is appreciated that specific functions, and accordingly utilities, of VGAM177 correlate with, and may be deduced from, the identity of the target genes which VGAM177 binds and inhibits, and the function of these target genes, as elaborated hereinbelow.

FLJ20413 (Accession NM_{—}017808) is a VGAM177 host target gene. FLJ20413 BINDING SITE is HOST TARGET binding site found in the 3′ untranslated region of mRNA encoded by FLJ20413, corresponding to a HOST TARGET binding site such as BINDING SITE I, BINDING SITE II or BINDING SITE III. Table 2 illustrates the complementarity of the nucleotide sequences of FLJ20413 BINDING SITE, designated SEQ ID:1753, to the nucleotide sequence of VGAM177 RNA, herein designated VGAM RNA, also designated SEQ ID:512.

A function of VGAM177 is therefore inhibition of FLJ20413 (Accession NM_{—}017808). Accordingly, utilities of VGAM177 include diagnosis, prevention and treatment of diseases and clinical conditions associated with FLJ20413. FIG. 1 further provides a conceptual description of a novel bioinformatically detected viral gene of the present invention, referred to here as Viral Genomic Address Messenger 178 (VGAM178) viral gene, which modulates expression of respective host target genes thereof, the function and utility of which host target genes is known in the art.

VGAM178 is a novel bioinformatically detected regulatory, non protein coding, viral micro RNA (miRNA) gene. The method by which VGAM178 was detected is described hereinabove with reference to FIGS. 1-8.

VGAM178 gene, herein designated VGAM GENE, is a viral gene contained in the genome of Vaccinia Virus. VGAM178 host target gene, herein designated VGAM HOST TARGET GENE, is a human gene contained in the human genome.

VGAM178 gene encodes a VGAM178 precursor RNA, herein designated VGAM PRECURSOR RNA. Similar to other miRNA genes, and unlike most ordinary genes, VGAM178 precursor RNA does not encode a protein. A nucleotide sequence identical or highly similar to the nucleotide sequence of VGAM178 precursor RNA is designated SEQ ID:164, and is provided hereinbelow with reference to the sequence listing part. Nucleotide sequence SEQ ID: 164 is located at position 93077 relative to the genome of Vaccinia Virus.

VGAM178 precursor RNA folds onto itself, forming VGAM178 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM178 folded precursor RNA into VGAM178 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM178 RNA is designated SEQ ID:513, and is provided hereinbelow with reference to the sequence listing part.

VGAM178 host target gene, herein designated VGAM HOST TARGET GENE, encodes a corresponding messenger RNA, VGAM178 host target RNA, herein designated VGAM HOST TARGET RNA. VGAM178 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM178 RNA, herein designated VGAM RNA, binds complementarily to one or more host target binding sites located in untranslated regions of VGAM178 host target RNA, herein designated VGAM HOST TARGET RNA. This complementary binding is due to the fact that the nucleotide sequence of VGAM178 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM178 RNA, herein designated VGAM RNA, may have a different number of host target binding sites in untranslated regions of a VGAM178 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM178 RNA, herein designated VGAM RNA, to host target binding sites on VGAM178 host target RNA, herein designated VGAM HOST TARGET RNA, such as BINDING SITE I, BINDING SITE II and BINDING SITE III, inhibits translation of VGAM178 host target RNA into VGAM178 host target protein, herein designated VGAM HOST TARGET PROTEIN. VGAM host target protein is therefore outlined by a broken line.

It is appreciated that VGAM178 host target gene, herein designated VGAM HOST TARGET GENE, in fact represents a plurality of VGAM178 host target genes. The mRNA of each one of this plurality of VGAM178 host target genes comprises one or more host target binding sites, each having a nucleotide sequence which is at least partly complementary to VGAM178 RNA, herein designated VGAM RNA, and which when bound by VGAM178 RNA causes inhibition of translation of respective one or more VGAM178 host target proteins.

It is further appreciated by one skilled in the art that the mode of translational inhibition illustrated by FIG. 1 with specific reference to translational inhibition exerted by VGAM178 gene, herein designated VGAM GENE, on one or more VGAM178 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM178 is inhibition of expression of host target genes, as part of a novel viral mechanism of attacking a host. Accordingly, utilities of VGAM178 include diagnosis, prevention and treatment of viral infection by Vaccinia Virus. Specific functions, and accordingly utilities, of VGAM178 correlate with, and may be deduced from, the identity of the host target genes which VGAM178 binds and inhibits, and the function of these host target genes, as elaborated hereinbelow.

Nucleotide sequences of the VGAM178 precursor RNA, herein designated VGAM PRECURSOR RNA, and of the ‘diced’ VGAM178 RNA, herein designated VGAM RNA, and a schematic representation of the secondary folding of VGAM178 folded precursor RNA, herein designated VGAM FOLDED PRECURSOR RNA, of VGAM178 are further described hereinbelow with reference to Table 1.

Nucleotide sequences of host target binding sites, such as BINDING SITE-I, BINDING SITE-II and BINDING SITE-III of FIG. 1, found on VGAM178 host target RNA, and schematic representation of the complementarity of each of these host target binding sites to VGAM178 RNA, herein designated VGAM RNA, are described hereinbelow with reference to Table 2.

As mentioned hereinabove with reference to FIG. 1, a function of VGAM178 gene, herein designated VGAM is inhibition of expression of VGAM178 target genes. It is appreciated that specific functions, and accordingly utilities, of VGAM178 correlate with, and may be deduced from, the identity of the target genes which VGAM178 binds and inhibits, and the function of these target genes, as elaborated hereinbelow.

C-reactive Protein, Pentraxin-related (CRP, Accession XM_{—}049673) is a VGAM178 host target gene. CRP BINDING SITE is HOST TARGET binding site found in the 3′ untranslated region of mRNA encoded by CRP, corresponding to a HOST TARGET binding site such as BINDING SITE I, BINDING SITE II or BINDING SITE III. Table 2 illustrates the complementarity of the nucleotide sequences of CRP BINDING SITE, designated SEQ ID:2920, to the nucleotide sequence of VGAM178 RNA, herein designated VGAM RNA, also designated SEQ ID:513.

A function of VGAM178 is therefore inhibition of C-reactive Protein, Pentraxin-related (CRP, Accession XM_{—}049673). Accordingly, utilities of VGAM178 include diagnosis, prevention and treatment of diseases and clinical conditions associated with CRP. DEAD/H (Asp-Glu-Ala-Asp/His) Box Polypeptide, Y Chromosome (DBY, Accession NM_{—}004660) is another VGAM178 host target gene. DBY BINDING SITE is HOST TARGET binding site found in the 3′ untranslated region of mRNA encoded by DBY, corresponding to a HOST TARGET binding site such as BINDING SITE I, BINDING SITE II or BINDING SITE III. Table 2 illustrates the complementarity of the nucleotide sequences of DBY BINDING SITE, designated SEQ ID:1140, to the nucleotide sequence of VGAM178 RNA, herein designated VGAM RNA, also designated SEQ ID:513.

Another function of VGAM178 is therefore inhibition of DEAD/H (Asp-Glu-Ala-Asp/His) Box Polypeptide, Y Chromosome (DBY, Accession NM_{—}004660), a gene which plays a key role in the spermatogenic process. Accordingly, utilities of VGAM178 include diagnosis, prevention and treatment of diseases and clinical conditions associated with DBY. The function of DBY has been established by previous studies. Foresta et al. (2000) reported a complete sequence map of the AZFa region (see OMIM Ref. No. 415000), the genomic structure of AZFa genes, and their deletion analysis in 173 infertile men with well-defined spermatogenic alterations. Deletions were found in 9 patients: DBY alone was deleted in 6, USP9Y only in 1, and 1 each with USP9Y-DBY or DBY-UTY missing. No patients solely lacked UTY. Patients lacking DBY exhibited either Sertoli cell-only syndrome or severe hypospermatogenesis. Expression analysis of AZFa genes and their X homologs revealed ubiquitous expression for all of them except DBY; a shorter DBY transcript was expressed only in testis. The authors suggested that DBY plays a key role in the spermatogenic process. Using COS cells to express candidate Y chromosome genes and mouse dendritic cells for antigenic presentation, Scott et al. (2000) determined that Dby expresses 2 major histocompatibility complex (MHC) class II-restricted minor histocompatibility HY determinants. No stimulatory activity was found with these epitopes in cells transfected with Smcy (OMIM Ref. No. 426000) or Uty, both of which express MHC class I-restricted epitopes.

Full details of the abovementioned studies are described in the following publications, the disclosure of which are hereby incorporated by reference:
Foresta, C.; Ferlin, A.; Moro, E.: Deletion and expression analysis of AZFa genes on the human Y chromosome revealed a major role for DBY in male infertility. Hum. Molec. Genet. 9: 1161-1169, 2000.; and
Scott, D.; Addey, C.; Ellis, P; James, E.; Mitchell, M. J.; Saut, N.; Jurcevic, S.; Simpson, E.: Dendritic cells permit identification of genes encoding MHC class II-restricted epitope.

Further studies establishing the function and utilities of DBY are found in John Hopkins OMIM database record ID 400010, and in sited publications numbered 199 and 2526 listed in the bibliography section hereinbelow, which are also hereby incorporated by reference. V-erb-b2 Erythroblastic Leukemia Viral Oncogene Homolog 2, Neuro/glioblastoma Derived Oncogene Homolog (avian) (ERBB2, Accession NM_{—}004448) is another VGAM178 host target gene. ERBB2 BINDING SITE is HOST TARGET binding site found in the 3′ untranslated region of mRNA encoded by ERBB2, corresponding to a HOST TARGET binding site such as BINDING SITE I, BINDING SITE II or BINDING SITE III. Table 2 illustrates the complementarity of the nucleotide sequences of ERBB2 BINDING SITE, designated SEQ ID:1115, to the nucleotide sequence of VGAM178 RNA, herein designated VGAM RNA, also designated SEQ ID:513.

Another function of VGAM178 is therefore inhibition of V-erb-b2 Erythroblastic Leukemia Viral Oncogene Homolog 2, Neuro/glioblastoma Derived Oncogene Homolog (avian) (ERBB2, Accession NM_{—}004448), a gene which Tyrosine kinase receptor. Accordingly, utilities of VGAM178 include diagnosis, prevention and treatment of diseases and clinical conditions associated with ERBB2. The function of ERBB2 has been established by previous studies. The oncogene originally called NEU was derived from rat neuro/glioblastoma cell lines. It encodes a tumor antigen, p185, which is serologically related to EGFR, the epidermal growth factor receptor (OMIM Ref. No. 131550). EGFR maps to chromosome 7. Yang-Feng et al. (1985) found, however, that the human homolog, which they designated NGL (to avoid confusion with neuraminidase, which is also symbolized NEU), maps to 17q12-q22 by in situ hybridization and to 17q21-qter in somatic cell hybrids. Thus, the SRO is 17q21-q22. Coussens et al. (1985) identified a potential cell surface receptor of the tyrosine kinase gene family and characterized it by cloning the gene. Its primary sequence is very similar to that of the human epidermal growth factor receptor. Because of the seemingly close relationship to the human EGF receptor, the authors called the gene HER2. By Southern blot analysis of somatic cell hybrid DNA and by in situ hybridization, the gene was assigned to 17q21-q22. This chromosomal location of the gene is coincident with the NEU oncogene, which suggests that the 2 genes may in fact be the same; indeed, sequencing indicates that they are identical (Francke, 1988). Van de Vijver et al. (1988) found a correlation between overexpression of NEU protein and the large-cell, comedo growth type of ductal carcinoma. They could find no correlation, however, with lymph-node status or tumor recurrence. Slamon et al. (1989) described the role of HER2/NEU in breast (OMIM Ref. No. 114480) and ovarian cancer (OMIM Ref. No. 167000), which together account for one-third of all cancers in women and approximately one-quarter of cancer-related deaths in females. The HER2 gene is amplified and HER2 is overexpressed in 25 to 30% of breast cancers, increasing the aggressiveness of the tumor. Slamon et al. (2001) found that herceptin increased the clinical benefit of first-line chemotherapy in metastatic breast cancer that overexpresses HER2. In a population-based case control study of the val655-to-ile polymorphism (164870.0001), Xie et al. (2000) found that the val allele was associated with an increased risk of breast cancer, particularly among younger women. Because of the significant ethnic differences in the incidence of breast cancer and other solid tumors, Ameyaw et al. (2002) undertook a study of 7 ethnic groups from 3 separate continents. The frequency of the val allele was highly variable between populations (1 to 24%). The continental African populations had a lower frequency than did the other subjects, corresponding with the lower incidence and lower risk of breast cancer in African women compared with Caucasian and African-American women. Animal model experiments lend further support to the function of ERBB2. An activated mutant form of ERBB2 is rarely found in human cancer. Instead, wildtype ERBB2 is overexpressed and/or amplified in 10 to 30% of breast cancers, where it correlates with chemoresistance and poor patient prognosis. Herceptin, a monoclonal antibody against ERBB2, is an effective treatment for a subset of patients with advanced breast cancer. Liu et al. (2002) used a transgenic mouse model with targeted aberrant overexpression of ERBB2 to determine whether genetic instability is associated with mammary tumorigenesis in vivo in the absence of heritable defects in known DNA maintenance genes.

It is appreciated that the abovementioned animal model for ERBB2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Coussens, L.; Yang-Feng, T. L.; Liao, Y.-C.; Chen, E.; Gray, A.; McGrath, J.; Seeburg, P. H.; Libermann, T. A.; Schlessinger, J.; Francke, U.; Levinson, A.; Ullrich, A.: Tyrosine kinase receptor with extensive homology to EGF receptor shares chromosomal location with NEU oncogene. Science 230: 1132-1139, 1985.; and
Liu, S.; Liu, W.; Jakubczak, J. L.; Erexson, G. L.; Tindall, K. R.; Chan, R.; Muller, W. J.; Adhya, S.; Garges, S.; Merlino, G.: Genetic instability favoring transversions associated w.

Further studies establishing the function and utilities of ERBB2 are found in John Hopkins OMIM database record ID 164870, and in sited publications numbered 399-400, 2668-478, 2789-494, 62 and 698-702 listed in the bibliography section hereinbelow, which are also hereby incorporated by reference. Integrin, Alpha 1 (ITGA1, Accession XM_{—}032902) is another VGAM178 host target gene. ITGA1 BINDING SITE is HOST TARGET binding site found in the 3′ untranslated region of mRNA encoded by ITGA1, corresponding to a HOST TARGET binding site such as BINDING SITE I, BINDING SITE II or BINDING SITE III. Table 2 illustrates the complementarity of the nucleotide sequences of ITGA1 BINDING SITE, designated SEQ ID:2660, to the nucleotide sequence of VGAM178 RNA, herein designated VGAM RNA, also designated SEQ ID:513.

Another function of VGAM178 is therefore inhibition of Integrin, Alpha 1 (ITGA1, Accession XM_{—}032902). Accordingly, utilities of VGAM178 include diagnosis, prevention and treatment of diseases and clinical conditions associated with ITGA1. Membrane-spanning 4-domains, Sub family A, Member 1 (MS4A1, Accession NM_{—}000139) is another VGAM178 host target gene. MS4A1 BINDING SITE is HOST TARGET binding site found in the 3′ untranslated region of mRNA encoded by MS4A1, corresponding to a HOST TARGET binding site such as BINDING SITE I, BINDING SITE II or BINDING SITE III. Table 2 illustrates the complementarity of the nucleotide sequences of MS4A1 BINDING SITE, designated SEQ ID:710, to the nucleotide sequence of VGAM178 RNA, herein designated VGAM RNA, also designated SEQ ID:513.

Another function of VGAM178 is therefore inhibition of Membrane-spanning 4-domains, Sub family A, Member 1 (MS4A1, Accession NM_{—}000139), a gene which may be involved in the regulation of b-cell activation and proliferation. Accordingly, utilities of VGAM178 include diagnosis, prevention and treatment of diseases and clinical conditions associated with MS4A1. The function of MS4A1 has been established by previous studies. B1, also known as CD20 or MS4A1, is a human B-lymphocyte surface molecule that is widely expressed during B-cell ontogeny, from early pre-B-cell developmental stages until final differentiation into plasma cells. Functional studies using monoclonal antibodies have shown that antibody binding to B1 inhibits B-cell proliferation caused by mitogens and inhibits B-cell differentiation. Tedder et al. (1988) described the primary structure of CD20. Tedder et al. (1989) showed that the CD20 gene is 16 kb long and contains 8 exons. Using in situ hybridization and Southern blotting of hybrid cell DNA, Tedder et al. (1989) showed that the CD20 gene is located on 11q12-q13. This localization places the CD20 gene near the site of the t(11;14)(q13;q32) translocation that is found in a subgroup of B-cell malignancies; see 151400. The CD20 gene was found to lie on the centromeric side of BCL1 (OMIM Ref. No. 168461) and to be separated from BCL1 by at least 50 kb of DNA. The proximal location of CD20 was indicated by the fact that it is not translocated to chromosome 14 in the translocation. It must be located between the centromere of chromosome 11 and the 3-prime end of BCL1. Szepetowski et al. (1993) studied amplification of the BCL1 region in breast cancer to map genes in the 11q13 band. CD20 was the most proximal of 13 genes located centromeric to BCL1 and was in the same group as CD5, PGA4 (OMIM Ref. No. 169720), and FTH1 (OMIM Ref. No. 134770). Distal to this cluster was a group of 3 genes, COX8 (OMIM Ref. No. 123870), PYGM (OMIM Ref. No. 232600), and SEA (OMIM Ref. No. 165110), of which the most proximal was COX8.

Full details of the abovementioned studies are described in the following publications, the disclosure of which are hereby incorporated by reference:
Szepetowski, P.; Perucca-Lostanlen, D.; Gaudray, P.: Mapping genes according to their amplification status in tumor cells: contribution to the map of 11q13. Genomics 16: 745-750, 1993.; and
Tedder, T. F.; Klejman, G.; Schlossman, S. F.; Saito, H.: Structure of the gene encoding the human B lymphocyte differentiation antigen CD20 (B1). J. Immun. 142: 2560-2568, 1989.

Further studies establishing the function and utilities of MS4A1 are found in John Hopkins OMIM database record ID 112210, and in sited publications numbered 104 and 1041-1043 listed in the bibliography section hereinbelow, which are also hereby incorporated by reference. ARSDR1 (Accession NM_{—}016026) is another VGAM178 host target gene. ARSDR1 BINDING SITE is HOST TARGET binding site found in the 3′ untranslated region of mRNA encoded by ARSDR1, corresponding to a HOST TARGET binding site such as BINDING SITE I, BINDING SITE II or BINDING SITE III. Table 2 illustrates the complementarity of the nucleotide sequences of ARSDR1 BINDING SITE, designated SEQ ID: 1656, to the nucleotide sequence of VGAM178 RNA, herein designated VGAM RNA, also designated SEQ ID:513.

Another function of VGAM178 is therefore inhibition of ARSDR1 (Accession NM_{—}016026). Accordingly, utilities of VGAM178 include diagnosis, prevention and treatment of diseases and clinical conditions associated with ARSDR1. ATPase, (Na+)/K+ Transporting, Beta 4 Polypeptide (ATP1B4, Accession NM_{—}012069) is another VGAM178 host target gene. ATP1B4 BINDING SITE is HOST TARGET binding site found in the 3′ untranslated region of mRNA encoded by ATP1B4, corresponding to a HOST TARGET binding site such as BINDING SITE I, BINDING SITE II or BINDING SITE III. Table 2 illustrates the complementarity of the nucleotide sequences of ATP1B4 BINDING SITE, designated SEQ ID: 1400, to the nucleotide sequence of VGAM178 RNA, herein designated VGAM RNA, also designated SEQ ID:513.

Another function of VGAM178 is therefore inhibition of ATPase, (Na+)/K+ Transporting, Beta 4 Polypeptide (ATP1B4, Accession NM_{—}012069). Accordingly, utilities of VGAM178 include diagnosis, prevention and treatment of diseases and clinical conditions associated with ATP1B4. FLJ00024 (Accession XM_{—}033361) is another VGAM178 host target gene. FLJ00024 BINDING SITE is HOST TARGET binding site found in the 3′ untranslated region of mRNA encoded by FLJ00024, corresponding to a HOST TARGET binding site such as BINDING SITE I, BINDING SITE II or BINDING SITE III. Table 2 illustrates the complementarity of the nucleotide sequences of FLJ00024 BINDING SITE, designated SEQ ID:2665, to the nucleotide sequence of VGAM178 RNA, herein designated VGAM RNA, also designated SEQ ID:513.

Another function of VGAM178 is therefore inhibition of FLJ00024 (Accession XM_{—}033361). Accordingly, utilities of VGAM178 include diagnosis, prevention and treatment of diseases and clinical conditions associated with FLJ00024. FLJ10493 (Accession NM_{—}018112) is another VGAM178 host target gene. FLJ10493 BINDING SITE is HOST TARGET binding site found in the 3′ untranslated region of mRNA encoded by FLJ10493, corresponding to a HOST TARGET binding site such as BINDING SITE I, BINDING SITE II or BINDING SITE III. Table 2 illustrates the complementarity of the nucleotide sequences of FLJ10493 BINDING SITE, designated SEQ ID:1782, to the nucleotide sequence of VGAM178 RNA, herein designated VGAM RNA, also designated SEQ ID:513.

Another function of VGAM178 is therefore inhibition of FLJ10493 (Accession NM_{—}018112). Accordingly, utilities of VGAM178 include diagnosis, prevention and treatment of diseases and clinical conditions associated with FLJ10493. FLJ11175 (Accession NM_{—}018349) is another VGAM178 host target gene. FLJ11175 BINDING SITE is HOST TARGET binding site found in the 3′ untranslated region of mRNA encoded by FLJ11175, corresponding to a HOST TARGET binding site such as BINDING SITE I, BINDING SITE II or BINDING SITE III. Table 2 illustrates the complementarity of the nucleotide sequences of FLJ11175 BINDING SITE, designated SEQ ID:1816, to the nucleotide sequence of VGAM178 RNA, herein designated VGAM RNA, also designated SEQ ID:513.

Another function of VGAM178 is therefore inhibition of FLJ11175 (Accession NM_{—}018349). Accordingly, utilities of VGAM178 include diagnosis, prevention and treatment of diseases and clinical conditions associated with FLJ11175. KIAA0977 (Accession NM_{—}014900) is another VGAM178 host target gene. KIAA0977 BINDING SITE is HOST TARGET binding site found in the 3′ untranslated region of mRNA encoded by KIAA0977, corresponding to a HOST TARGET binding site such as BINDING SITE I, BINDING SITE II or BINDING SITE III. Table 2 illustrates the complementarity of the nucleotide sequences of KIAA0977 BINDING SITE, designated SEQ ID:1578, to the nucleotide sequence of VGAM178 RNA, herein designated VGAM RNA, also designated SEQ ID:513.

Another function of VGAM178 is therefore inhibition of KIAA0977 (Accession NM_{—}014900). Accordingly, utilities of VGAM178 include diagnosis, prevention and treatment of diseases and clinical conditions associated with KIAA0977. Thyroid Hormone Receptor Interactor 3 (TRIP3, Accession XM_{—}085661) is another VGAM178 host target gene. TRIP3 BINDING SITE is HOST TARGET binding site found in the 3′ untranslated region of mRNA encoded by TRIP3, corresponding to a HOST TARGET binding site such as BINDING SITE I, BINDING SITE II or BINDING SITE III. Table 2 illustrates the complementarity of the nucleotide sequences of TRIP3 BINDING SITE, designated SEQ ID:3102, to the nucleotide sequence of VGAM178 RNA, herein designated VGAM RNA, also designated SEQ ID:513.

Another function of VGAM178 is therefore inhibition of Thyroid Hormone Receptor Interactor 3 (TRIP3, Accession XM_{—}085661). Accordingly, utilities of VGAM178 include diagnosis, prevention and treatment of diseases and clinical conditions associated with TRIP3. LOC149117 (Accession XM_{—}097587) is another VGAM178 host target gene. LOC149117 BINDING SITE is HOST TARGET binding site found in the 3′ untranslated region of mRNA encoded by LOC149117, corresponding to a HOST TARGET binding site such as BINDING SITE I, BINDING SITE II or BINDING SITE III. Table 2 illustrates the complementarity of the nucleotide sequences of LOC149117 BINDING SITE, designated SEQ ID:3307, to the nucleotide sequence of VGAM178 RNA, herein designated VGAM RNA, also designated SEQ ID:513.

Another function of VGAM178 is therefore inhibition of LOC149117 (Accession XM_{—}097587). Accordingly, utilities of VGAM178 include diagnosis, prevention and treatment of diseases and clinical conditions associated with LOC149117. LOC196993 (Accession XM_{—}116971) is another VGAM178 host target gene. LOC196993 BINDING SITE is HOST TARGET binding site found in the 5′ untranslated region of mRNA encoded by LOC196993, corresponding to a HOST TARGET binding site such as BINDING SITE I, BINDING SITE II or BINDING SITE III. Table 2 illustrates the complementarity of the nucleotide sequences of LOC196993 BINDING SITE, designated SEQ ID:3471, to the nucleotide sequence of VGAM178 RNA, herein designated VGAM RNA, also designated SEQ ID:513.

Another function of VGAM178 is therefore inhibition of LOC196993 (Accession XM_{—}116971). Accordingly, utilities of VGAM178 include diagnosis, prevention and treatment of diseases and clinical conditions associated with LOC196993. LOC56965 (Accession NM_{—}020213) is another VGAM178 host target gene. LOC56965 BINDING SITE is HOST TARGET binding site found in the 5′ untranslated region of mRNA encoded by LOC56965, corresponding to a HOST TARGET binding site such as BINDING SITE I, BINDING SITE II or BINDING SITE III. Table 2 illustrates the complementarity of the nucleotide sequences of LOC56965 BINDING SITE, designated SEQ ID:1900, to the nucleotide sequence of VGAM178 RNA, herein designated VGAM RNA, also designated SEQ ID:513.

Another function of VGAM178 is therefore inhibition of LOC56965 (Accession NM_{—}020213). Accordingly, utilities of VGAM178 include diagnosis, prevention and treatment of diseases and clinical conditions associated with LOC56965. FIG. 1 further provides a conceptual description of a novel bioinformatically detected viral gene of the present invention, referred to here as Viral Genomic Address Messenger 179 (VGAM179) viral gene, which modulates expression of respective host target genes thereof, the function and utility of which host target genes is known in the art.

VGAM179 is a novel bioinformatically detected regulatory, non protein coding, viral micro RNA (miRNA) gene. The method by which VGAM179 was detected is described hereinabove with reference to FIGS. 1-8.

VGAM179 gene, herein designated VGAM GENE, is a viral gene contained in the genome of Vaccinia Virus. VGAM179 host target gene, herein designated VGAM HOST TARGET GENE, is a human gene contained in the human genome.

VGAM179 gene encodes a VGAM179 precursor RNA, herein designated VGAM PRECURSOR RNA. Similar to other miRNA genes, and unlike most ordinary genes, VGAM179 precursor RNA does not encode a protein. A nucleotide sequence identical or highly similar to the nucleotide sequence of VGAM179 precursor RNA is designated SEQ ID:165, and is provided hereinbelow with reference to the sequence listing part. Nucleotide sequence SEQ ID: 165 is located at position 92769 relative to the genome of Vaccinia Virus.

VGAM179 precursor RNA folds onto itself, forming VGAM179 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM179 folded precursor RNA into VGAM179 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM179 RNA is designated SEQ ID:514, and is provided hereinbelow with reference to the sequence listing part.

VGAM179 host target gene, herein designated VGAM HOST TARGET GENE, encodes a corresponding messenger RNA, VGAM179 host target RNA, herein designated VGAM HOST TARGET RNA. VGAM179 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM179 RNA, herein designated VGAM RNA, binds complementarily to one or more host target binding sites located in untranslated regions of VGAM179 host target RNA, herein designated VGAM HOST TARGET RNA. This complementary binding is due to the fact that the nucleotide sequence of VGAM179 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM179 RNA, herein designated VGAM RNA, may have a different number of host target binding sites in untranslated regions of a VGAM179 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM179 RNA, herein designated VGAM RNA, to host target binding sites on VGAM179 host target RNA, herein designated VGAM HOST TARGET RNA, such as BINDING SITE I, BINDING SITE II and BINDING SITE III, inhibits translation of VGAM179 host target RNA into VGAM179 host target protein, herein designated VGAM HOST TARGET PROTEIN. VGAM host target protein is therefore outlined by a broken line.

It is appreciated that VGAM179 host target gene, herein designated VGAM HOST TARGET GENE, in fact represents a plurality of VGAM179 host target genes. The mRNA of each one of this plurality of VGAM179 host target genes comprises one or more host target binding sites, each having a nucleotide sequence which is at least partly complementary to VGAM179 RNA, herein designated VGAM RNA, and which when bound by VGAM179 RNA causes inhibition of translation of respective one or more VGAM179 host target proteins.

It is further appreciated by one skilled in the art that the mode of translational inhibition illustrated by FIG. 1 with specific reference to translational inhibition exerted by VGAM179 gene, herein designated VGAM GENE, on one or more VGAM179 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM179 is inhibition of expression of host target genes, as part of a novel viral mechanism of attacking a host. Accordingly, utilities of VGAM179 include diagnosis, prevention and treatment of viral infection by Vaccinia Virus. Specific functions, and accordingly utilities, of VGAM179 correlate with, and may be deduced from, the identity of the host target genes which VGAM179 binds and inhibits, and the function of these host target genes, as elaborated hereinbelow.

Nucleotide sequences of the VGAM179 precursor RNA, herein designated VGAM PRECURSOR RNA, and of the ‘diced’ VGAM179 RNA, herein designated VGAM RNA, and a schematic representation of the secondary folding of VGAM179 folded precursor RNA, herein designated VGAM FOLDED PRECURSOR RNA, of VGAM179 are further described hereinbelow with reference to Table 1.

Nucleotide sequences of host target binding sites, such as BINDING SITE-I, BINDING SITE-II and BINDING SITE-III of FIG. 1, found on VGAM179 host target RNA, and schematic representation of the complementarity of each of these host target binding sites to VGAM179 RNA, herein designated VGAM RNA, are described hereinbelow with reference to Table 2.

As mentioned hereinabove with reference to FIG. 1, a function of VGAM179 gene, herein designated VGAM is inhibition of expression of VGAM179 target genes. It is appreciated that specific functions, and accordingly utilities, of VGAM179 correlate with, and may be deduced from, the identity of the target genes which VGAM179 binds and inhibits, and the function of these target genes, as elaborated hereinbelow.

Nuclear Mitotic Apparatus Protein 1 (NUMA1, Accession XM_{—}167853) is a VGAM179 host target gene. NUMAL BINDING SITE is HOST TARGET binding site found in the 3′ untranslated region of mRNA encoded by NUMA1, corresponding to a HOST TARGET binding site such as BINDING SITE I, BINDING SITE II or BINDING SITE III. Table 2 illustrates the complementarity of the nucleotide sequences of NUMAL BINDING SITE, designated SEQ ID:3604, to the nucleotide sequence of VGAM179 RNA, herein designated VGAM RNA, also designated SEQ ID:514.

A function of VGAM179 is therefore inhibition of Nuclear Mitotic Apparatus Protein 1 (NUMA1, Accession XM_{—}167853), a gene which is nuclear mitotic apparatus protein. Accordingly, utilities of VGAM179 include diagnosis, prevention and treatment of diseases and clinical conditions associated with NUMAL. The function of NUMAL has been established by previous studies. The NuMA protein was one of the first to be described as a cell cycle-related protein based on a distinct immunofluorescent staining pattern: in interphase, NuMA is present throughout the nucleus, and in mitosis, it localizes to the spindle apparatus (Lydersen and Pettijohn, 1980). Some patients with autoimmune disease have antibodies directed against the NuMA protein. The full-length NUMA cDNA (Compton et al., 1992; Yang et al., 1992) predicts a protein with the largest known coiled-coil region in a protein. By fluorescence in situ hybridization, Sparks et al. (1993) demonstrated that the NUMAL gene is present in single copy and located on 11q13. Acute promyelocytic leukemia (APL) is uniquely associated with chromosomal translocations that disrupt the gene encoding the retinoic acid receptor, RARA (OMIM Ref. No. 180240). In more than 99% of cases, this disruption results in the formation of a fusion of the RARA gene with the PML gene (OMIM Ref. No. 102578). In rare variants of APL, the RARA gene has been found to be fused to 1 of 2 other genes, PLZF (OMIM Ref. No. 176797) and NPM (OMIM Ref. No. 164040). Although RARA dysregulation is evidently important in APL, the role of the various fusion partners is unclear. Wells et al. (1997) characterized a fourth APL gene fusion, which linked exons encoding the retinoic acid and DNA-binding domains of RARA to 5-prime exons of NUMAL. The NUMA/RARA fusion protein existed in sheet-like nuclear aggregates with which normal NUMA partly colocalized. In contrast to t(15;17) APL (the usual variety) the intracellular distribution of PML was normal in these cells. Wells et al. (1997) suggested that interference with retinoid signaling, and not disruption of PML organization, is essential to the APL phenotype. Their work implicated for the first time an element of the mitotic apparatus in the molecular pathogenesis of human malignancy. The proband of their study was a Caucasian male first seen at 6 months of age for investigation of multiple cutaneous lesions. Despite this unusual clinical presentation, peripheral blood morphology and cell-surface immunophenotype were typical of APL. Routine analysis of diagnostic bone marrow revealed a clonal cytogenetic abnormality, t(11;17)(q13;q21). The patient was treated with all-trans retinoic acid and achieved complete remission; he remained in morphologic remission 38 months after autologous bone marrow transplantation.

Full details of the abovementioned studies are described in the following publications, the disclosure of which are hereby incorporated by reference:
Lydersen, B. K.; Pettijohn, D. E.: Human-specific nuclear protein that associates with the polar region of the mitotic apparatus: distribution in a human/hamster hybrid cell. Cell 22: 489-499, 1980.; and
Wells, R. A.; Catzavelos, C.; Kamel-Reid, S.: Fusion of retinoic acid receptor alpha to NuMA, the nuclear mitotic apparatus protein, by a variant translocation in acute promyelocytic 1.

Further studies establishing the function and utilities of NUMAL are found in John Hopkins OMIM database record ID 164009, and in sited publications numbered 504-510 listed in the bibliography section hereinbelow, which are also hereby incorporated by reference. Solute Carrier Family 15 (oligopeptide transporter), Member 1 (SLC15A1, Accession NM_{—}005073) is another VGAM179 host target gene. SLC15A1 BINDING SITE is HOST TARGET binding site found in the 3′ untranslated region of mRNA encoded by SLC15A1, corresponding to a HOST TARGET binding site such as BINDING SITE I, BINDING SITE II or BINDING SITE III. Table 2 illustrates the complementarity of the nucleotide sequences of SLC15A1 BINDING SITE, designated SEQ ID:1182, to the nucleotide sequence of VGAM179 RNA, herein designated VGAM RNA, also designated SEQ ID:514.

Another function of VGAM179 is therefore inhibition of Solute Carrier Family 15 (oligopeptide transporter), Member 1 (SLC15A1, Accession NM_{—}005073), a gene which is a H(+)-coupled peptide transporter. Accordingly, utilities of VGAM179 include diagnosis, prevention and treatment of diseases and clinical conditions associated with SLC15A1. The function of SLC15A1 has been established by previous studies. In mammalian small intestine, the proton-coupled peptide transporter is responsible for the absorption of small peptides arising from digestion of dietary proteins. Fei et al. (1994) isolated a cDNA clone encoding a hydrogen ion/peptide cotransporter from a rabbit intestinal cDNA library. Liang et al. (1995) screened a human intestinal cDNA library with a probe derived from the rabbit cotransporter cDNA and identified a cDNA which, when expressed in HeLa cells or in Xenopus laevis oocytes, induced proton-dependent peptide transport activity. The predicted protein consisted of 708 amino acids with 12 membrane-spanning domains and 2 putative sites for protein kinase C-dependent phosphorylation. The cDNA-induced transport process accepted dipeptides, tripeptides, and amino beta-lactam antibiotics as substrates, but could not transport free amino acids. The human cotransporter showed 81% identity and 92% similarity to the rabbit cotransporter, but showed only a weak homology to the proton-coupled peptide transport proteins present in bacteria and yeast. By analysis of somatic cell hybrids and by isotopic in situ hybridization, Liang et al. (1995) mapped the human gene to 13q33-q34. Adibi (1997) reviewed the biology and function of the human intestinal oligopeptide transporter, which he symbolized PEPT1. Studies indicated that it transports dipeptides and tripeptides but not free amino acids or peptides with more than 3 amino acid residues and that its driving force for uphill transport requires proton binding and presence of an inside-negative membrane potential. A membrane protein, HTP1, which appeared to be associated with the oligopeptide transporter, had also been cloned. Adibi (1997) pointed out the importance of the transporter in nutritional and pharmacologic therapies; for example, it has allowed the use of oligopeptides as a source of nitrogen for enteral feeding and the use of the oral route for delivery of peptidomimetic drugs such as beta-lactam antibiotics.

Full details of the abovementioned studies are described in the following publications, the disclosure of which are hereby incorporated by reference:
Liang, R.; Fei, Y.-J.; Prasad, P. D.; Ramamoorthy, S.; Han, H.; Yang-Feng, T. L.; Hediger, M. A.; Ganaphthy, V.; Leibach, F. H.: Human intestinal H(+)/peptide cotransporter: cloning, functional expression, and chromosomal localization. J. Biol. Chem. 270: 6456-6463, 1995.; and
Adibi, S. A.: The oligopeptide transporter (Pept-1) in human intestine: biology and function. Gastroenterology 113: 332-340, 1997.

Further studies establishing the function and utilities of SLC15A1 are found in John Hopkins OMIM database record ID 600544, and in sited publications numbered 1789-1791 listed in the bibliography section hereinbelow, which are also hereby incorporated by reference. CCR4—NOT Transcription Complex, Subunit 3 (CNOT3, Accession NM_{—}014516) is another VGAM179 host target gene. CNOT3 BINDING SITE is HOST TARGET binding site found in the 5′ untranslated region of mRNA encoded by CNOT3, corresponding to a HOST TARGET binding site such as BINDING SITE I, BINDING SITE II or BINDING SITE III. Table 2 illustrates the complementarity of the nucleotide sequences of CNOT3 BINDING SITE, designated SEQ ID: 1505, to the nucleotide sequence of VGAM179 RNA, herein designated VGAM RNA, also designated SEQ ID:514.

Another function of VGAM179 is therefore inhibition of CCR4—NOT Transcription Complex, Subunit 3 (CNOT3, Accession NM_{—}014516). Accordingly, utilities of VGAM179 include diagnosis, prevention and treatment of diseases and clinical conditions associated with CNOT3. FLJ10898 (Accession XM_{—}002486) is another VGAM179 host target gene. FLJ10898 BINDING SITE is HOST TARGET binding site found in the 5′ untranslated region of mRNA encoded by FLJ10898, corresponding to a HOST TARGET binding site such as BINDING SITE I, BINDING SITE II or BINDING SITE III. Table 2 illustrates the complementarity of the nucleotide sequences of FLJ10898 BINDING SITE, designated SEQ ID:2527, to the nucleotide sequence of VGAM179 RNA, herein designated VGAM RNA, also designated SEQ ID:514.

Another function of VGAM179 is therefore inhibition of FLJ10898 (Accession XM_{—}002486). Accordingly, utilities of VGAM179 include diagnosis, prevention and treatment of diseases and clinical conditions associated with FLJ10898. FLJ14686 (Accession NM_{—}032825) is another VGAM179 host target gene. FLJ14686 BINDING SITE is HOST TARGET binding site found in the 3′ untranslated region of mRNA encoded by FLJ14686, corresponding to a HOST TARGET binding site such as BINDING SITE I, BINDING SITE II or BINDING SITE III. Table 2 illustrates the complementarity of the nucleotide sequences of FLJ14686 BINDING SITE, designated SEQ ID:2286, to the nucleotide sequence of VGAM179 RNA, herein designated VGAM RNA, also designated SEQ ID:514.

Another function of VGAM179 is therefore inhibition of FLJ14686 (Accession NM_{—}032825). Accordingly, utilities of VGAM179 include diagnosis, prevention and treatment of diseases and clinical conditions associated with FLJ14686. HDAC9-PENDING (Accession NM_{—}014707) is another VGAM179 host target gene. HDAC9-PENDING BINDING SITE is HOST TARGET binding site found in the 3′ untranslated region of mRNA encoded by HDAC9-PENDING, corresponding to a HOST TARGET binding site such as BINDING SITE I, BINDING SITE II or BINDING SITE III. Table 2 illustrates the complementarity of the nucleotide sequences of HDAC9-PENDING BINDING SITE, designated SEQ ID: 1526, to the nucleotide sequence of VGAM179 RNA, herein designated VGAM RNA, also designated SEQ ID:514.

Another function of VGAM179 is therefore inhibition of HDAC9-PENDING (Accession NM_{—}014707). Accordingly, utilities of VGAM179 include diagnosis, prevention and treatment of diseases and clinical conditions associated with HDAC9-PENDING. ZAK (Accession NM_{—}133646) is another VGAM179 host target gene. ZAK BINDING SITE is HOST TARGET binding site found in the 3′ untranslated region of mRNA encoded by ZAK, corresponding to a HOST TARGET binding site such as BINDING SITE I, BINDING SITE II or BINDING SITE III. Table 2 illustrates the complementarity of the nucleotide sequences of ZAK BINDING SITE, designated SEQ ID:2427, to the nucleotide sequence of VGAM179 RNA, herein designated VGAM RNA, also designated SEQ ID:514.

Another function of VGAM179 is therefore inhibition of ZAK (Accession NM_{—}133646). Accordingly, utilities of VGAM179 include diagnosis, prevention and treatment of diseases and clinical conditions associated with ZAK. LOC119548 (Accession XM_{—}058404) is another VGAM179 host target gene. LOC119548 BINDING SITE is HOST TARGET binding site found in the 5′ untranslated region of mRNA encoded by LOC119548, corresponding to a HOST TARGET binding site such as BINDING SITE I, BINDING SITE II or BINDING SITE III. Table 2 illustrates the complementarity of the nucleotide sequences of LOC119548 BINDING SITE, designated SEQ ID:2984, to the nucleotide sequence of VGAM179 RNA, herein designated VGAM RNA, also designated SEQ ID:514.

Another function of VGAM179 is therefore inhibition of LOC119548 (Accession XM_{—}058404). Accordingly, utilities of VGAM179 include diagnosis, prevention and treatment of diseases and clinical conditions associated with LOC119548. LOC153937 (Accession XM_{—}087813) is another VGAM179 host target gene. LOC153937 BINDING SITE is HOST TARGET binding site found in the 5′ untranslated region of mRNA encoded by LOC153937, corresponding to a HOST TARGET binding site such as BINDING SITE I, BINDING SITE II or BINDING SITE III. Table 2 illustrates the complementarity of the nucleotide sequences of LOC153937 BINDING SITE, designated SEQ ID:3185, to the nucleotide sequence of VGAM179 RNA, herein designated VGAM RNA, also designated SEQ ID:514.

Another function of VGAM179 is therefore inhibition of LOC153937 (Accession XM_{—}087813). Accordingly, utilities of VGAM179 include diagnosis, prevention and treatment of diseases and clinical conditions associated with LOC153937. LOC221895 (Accession XM_{—}166511) is another VGAM179 host target gene. LOC221895 BINDING SITE is HOST TARGET binding site found in the 3′ untranslated region of mRNA encoded by LOC221895, corresponding to a HOST TARGET binding site such as BINDING SITE I, BINDING SITE II or BINDING SITE III. Table 2 illustrates the complementarity of the nucleotide sequences of LOC221895 BINDING SITE, designated SEQ ID:3568, to the nucleotide sequence of VGAM179 RNA, herein designated VGAM RNA, also designated SEQ ID:514.

Another function of VGAM179 is therefore inhibition of LOC221895 (Accession XM_{—}166511). Accordingly, utilities of VGAM179 include diagnosis, prevention and treatment of diseases and clinical conditions associated with LOC221895. FIG. 1 further provides a conceptual description of a novel bioinformatically detected viral gene of the present invention, referred to here as Viral Genomic Address Messenger 180 (VGAM180) viral gene, which modulates expression of respective host target genes thereof, the function and utility of which host target genes is known in the art.

VGAM180 is a novel bioinformatically detected regulatory, non protein coding, viral micro RNA (miRNA) gene. The method by which VGAM180 was detected is described hereinabove with reference to FIGS. 1-8.

VGAM180 gene, herein designated VGAM GENE, is a viral gene contained in the genome of Vaccinia Virus. VGAM180 host target gene, herein designated VGAM HOST TARGET GENE, is a human gene contained in the human genome.

VGAM180 gene encodes a VGAM180 precursor RNA, herein designated VGAM PRECURSOR RNA. Similar to other miRNA genes, and unlike most ordinary genes, VGAM180 precursor RNA does not encode a protein. A nucleotide sequence identical or highly similar to the nucleotide sequence of VGAM180 precursor RNA is designated SEQ ID:166, and is provided hereinbelow with reference to the sequence listing part. Nucleotide sequence SEQ ID: 166 is located at position 93665 relative to the genome of Vaccinia Virus.

VGAM180 precursor RNA folds onto itself, forming VGAM180 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM180 folded precursor RNA into VGAM180 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM180 RNA is designated SEQ ID:515, and is provided hereinbelow with reference to the sequence listing part.

VGAM180 host target gene, herein designated VGAM HOST TARGET GENE, encodes a corresponding messenger RNA, VGAM180 host target RNA, herein designated VGAM HOST TARGET RNA. VGAM180 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM180 RNA, herein designated VGAM RNA, binds complementarily to one or more host target binding sites located in untranslated regions of VGAM180 host target RNA, herein designated VGAM HOST TARGET RNA. This complementary binding is due to the fact that the nucleotide sequence of VGAM180 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM180 RNA, herein designated VGAM RNA, may have a different number of host target binding sites in untranslated regions of a VGAM180 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM180 RNA, herein designated VGAM RNA, to host target binding sites on VGAM180 host target RNA, herein designated VGAM HOST TARGET RNA, such as BINDING SITE I, BINDING SITE II and BINDING SITE III, inhibits translation of VGAM180 host target RNA into VGAM180 host target protein, herein designated VGAM HOST TARGET PROTEIN. VGAM host target protein is therefore outlined by a broken line.

It is appreciated that VGAM180 host target gene, herein designated VGAM HOST TARGET GENE, in fact represents a plurality of VGAM180 host target genes. The mRNA of each one of this plurality of VGAM180 host target genes comprises one or more host target binding sites, each having a nucleotide sequence which is at least partly complementary to VGAM180 RNA, herein designated VGAM RNA, and which when bound by VGAM180 RNA causes inhibition of translation of respective one or more VGAM180 host target proteins.

It is further appreciated by one skilled in the art that the mode of translational inhibition illustrated by FIG. 1 with specific reference to translational inhibition exerted by VGAM180 gene, herein designated VGAM GENE, on one or more VGAM180 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM180 is inhibition of expression of host target genes, as part of a novel viral mechanism of attacking a host. Accordingly, utilities of VGAM180 include diagnosis, prevention and treatment of viral infection by Vaccinia Virus. Specific functions, and accordingly utilities, of VGAM180 correlate with, and may be deduced from, the identity of the host target genes which VGAM180 binds and inhibits, and the function of these host target genes, as elaborated hereinbelow.

Nucleotide sequences of the VGAM180 precursor RNA, herein designated VGAM PRECURSOR RNA, and of the ‘diced’ VGAM180 RNA, herein designated VGAM RNA, and a schematic representation of the secondary folding of VGAM180 folded precursor RNA, herein designated VGAM FOLDED PRECURSOR RNA, of VGAM180 are further described hereinbelow with reference to Table 1.

Nucleotide sequences of host target binding sites, such as BINDING SITE-I, BINDING SITE-II and BINDING SITE-III of FIG. 1, found on VGAM180 host target RNA, and schematic representation of the complementarity of each of these host target binding sites to VGAM180 RNA, herein designated VGAM RNA, are described hereinbelow with reference to Table 2.

As mentioned hereinabove with reference to FIG. 1, a function of VGAM180 gene, herein designated VGAM is inhibition of expression of VGAM180 target genes. It is appreciated that specific functions, and accordingly utilities, of VGAM180 correlate with, and may be deduced from, the identity of the target genes which VGAM180 binds and inhibits, and the function of these target genes, as elaborated hereinbelow.

B-cell Linker (BLNK, Accession NM_{—}013314) is a VGAM180 host target gene. BLNK BINDING SITE is HOST TARGET binding site found in the 5′ untranslated region of mRNA encoded by BLNK, corresponding to a HOST TARGET binding site such as BINDING SITE I, BINDING SITE II or BINDING SITE III. Table 2 illustrates the complementarity of the nucleotide sequences of BLNK BINDING SITE, designated SEQ ID: 1441, to the nucleotide sequence of VGAM180 RNA, herein designated VGAM RNA, also designated SEQ ID:515.

A function of VGAM180 is therefore inhibition of B-cell Linker (BLNK, Accession NM_{—}013314), a gene which is a component of the BCR transducer complex, promotes B-cell development. Accordingly, utilities of VGAM180 include diagnosis, prevention and treatment of diseases and clinical conditions associated with BLNK. The function of BLNK has been established by previous studies. In evaluating patients with absent B cells, Minegishi et al. (1999) identified a male with a homozygous splice defect in the gene encoding the cytoplasmic adaptor protein BLNK (604515.0001). Although this patient had normal numbers of pro-B cells, he had no pre-B cells or mature B cells, indicating that BLNK plays a critical role in orchestrating the pro-B cell to pre-B cell transition. The immune system and overall growth and development were otherwise normal in this patient, suggesting that BLNK function is highly specific. The patient had developed recurrent otitis at 8 months of age; after 2 episodes of pneumonia, he was evaluated for immunodeficiency at 16 months of age. At that time, he had no detectable serum IgG, IgM, or IgA, and he had less than 1% B cells in the peripheral circulation. He was started on gammaglobulin replacement, and between 2 and 20 years of age he did well except for chronic otitis and sinusitis, hepatitis C acquired from intravenous gammaglobulin, and an episode of protein-losing enteropathy in adolescence. Animal model experiments lend further support to the function of BLNK. Pappu et al. (1999) generated mice deficient in BLNK by targeted disruption. B-cell development in BLNK −/− mice was blocked at the transition from B220+CD43+ progenitor B to B220+CD43− precursor B cells. Only a small percentage of IgM M++, but not mature IgM(lo)IgD(hi), B cells were detected in the periphery. Pappu et al. (1999) concluded that BLNK is an essential component of the B-cell receptor signaling pathways and is required to promote B-cell development.

It is appreciated that the abovementioned animal model for BLNK is acknowledged by those skilled in the art as a scientifically valid animal model, as can be further appreciated from the publications sited hereinbelow.

Full details of the abovementioned studies are described in the following publications, the disclosure of which are hereby incorporated by reference:
Minegishi, Y.; Rohrer, J.; Coustan-Smith, E.; Lederman, H. M.; Pappu, R.; Campana, D.; Chan, A. C.; Conley, M. E.: An essential role for BLNK in human B cell development. Science 286: 1954-1957, 1999.; and
Pappu, R.; Cheng, A. M.; Li, B.; Gong, Q.; Chiu, C.; Griffin, N.; White, M.; Sleckman, B. P.; Chan, A. C.: Requirement for B cell linker protein (BLNK) in B cell development. Science.

Further studies establishing the function and utilities of BLNK are found in John Hopkins OMIM database record ID 604515, and in sited publications numbered 1234-123 and 1067-1068 listed in the bibliography section hereinbelow, which are also hereby incorporated by reference. FLJ20152 (Accession NM_{—}019000) is another VGAM180 host target gene. FLJ20152 BINDING SITE is HOST TARGET binding site found in the 3′ untranslated region of mRNA encoded by FLJ20152, corresponding to a HOST TARGET binding site such as BINDING SITE I, BINDING SITE II or BINDING SITE III. Table 2 illustrates the complementarity of the nucleotide sequences of FLJ20152 BINDING SITE, designated SEQ ID:1870, to the nucleotide sequence of VGAM180 RNA, herein designated VGAM RNA, also designated SEQ ID:515.

Another function of VGAM180 is therefore inhibition of FLJ20152 (Accession NM_{—}019000). Accordingly, utilities of VGAM180 include diagnosis, prevention and treatment of diseases and clinical conditions associated with FLJ20152. FIG. 1 further provides a conceptual description of a novel bioinformatically detected viral gene of the present invention, referred to here as Viral Genomic Address Messenger 181 (VGAM181) viral gene, which modulates expression of respective host target genes thereof, the function and utility of which host target genes is known in the art.

VGAM181 is a novel bioinformatically detected regulatory, non protein coding, viral micro RNA (miRNA) gene. The method by which VGAM181 was detected is described hereinabove with reference to FIGS. 1-8.

VGAM181 gene, herein designated VGAM GENE, is a viral gene contained in the genome of Vaccinia Virus. VGAM181 host target gene, herein designated VGAM HOST TARGET GENE, is a human gene contained in the human genome.

VGAM181 gene encodes a VGAM181 precursor RNA, herein designated VGAM PRECURSOR RNA. Similar to other miRNA genes, and unlike most ordinary genes, VGAM181 precursor RNA does not encode a protein. A nucleotide sequence identical or highly similar to the nucleotide sequence of VGAM181 precursor RNA is designated SEQ ID:167, and is provided hereinbelow with reference to the sequence listing part. Nucleotide sequence SEQ ID: 167 is located at position 96078 relative to the genome of Vaccinia Virus.

VGAM181 precursor RNA folds onto itself, forming VGAM181 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM181 folded precursor RNA into VGAM181 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM181 RNA is designated SEQ ID:516, and is provided hereinbelow with reference to the sequence listing part.

VGAM181 host target gene, herein designated VGAM HOST TARGET GENE, encodes a corresponding messenger RNA, VGAM181 host target RNA, herein designated VGAM HOST TARGET RNA. VGAM181 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM181 RNA, herein designated VGAM RNA, binds complementarily to one or more host target binding sites located in untranslated regions of VGAM181 host target RNA, herein designated VGAM HOST TARGET RNA. This complementary binding is due to the fact that the nucleotide sequence of VGAM181 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM181 RNA, herein designated VGAM RNA, may have a different number of host target binding sites in untranslated regions of a VGAM181 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM181 RNA, herein designated VGAM RNA, to host target binding sites on VGAM181 host target RNA, herein designated VGAM HOST TARGET RNA, such as BINDING SITE I, BINDING SITE II and BINDING SITE III, inhibits translation of VGAM181 host target RNA into VGAM181 host target protein, herein designated VGAM HOST TARGET PROTEIN. VGAM host target protein is therefore outlined by a broken line.

It is appreciated that VGAM181 host target gene, herein designated VGAM HOST TARGET GENE, in fact represents a plurality of VGAM181 host target genes. The mRNA of each one of this plurality of VGAM181 host target genes comprises one or more host target binding sites, each having a nucleotide sequence which is at least partly complementary to VGAM181 RNA, herein designated VGAM RNA, and which when bound by VGAM181 RNA causes inhibition of translation of respective one or more VGAM181 host target proteins.

It is further appreciated by one skilled in the art that the mode of translational inhibition illustrated by FIG. 1 with specific reference to translational inhibition exerted by VGAM181 gene, herein designated VGAM GENE, on one or more VGAM181 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM181 is inhibition of expression of host target genes, as part of a novel viral mechanism of attacking a host. Accordingly, utilities of VGAM181 include diagnosis, prevention and treatment of viral infection by Vaccinia Virus. Specific functions, and accordingly utilities, of VGAM181 correlate with, and may be deduced from, the identity of the host target genes which VGAM181 binds and inhibits, and the function of these host target genes, as elaborated hereinbelow.

Nucleotide sequences of the VGAM181 precursor RNA, herein designated VGAM PRECURSOR RNA, and of the ‘diced’ VGAM181 RNA, herein designated VGAM RNA, and a schematic representation of the secondary folding of VGAM181 folded precursor RNA, herein designated VGAM FOLDED PRECURSOR RNA, of VGAM181 are further described hereinbelow with reference to Table 1.

Nucleotide sequences of host target binding sites, such as BINDING SITE-I, BINDING SITE-II and BINDING SITE-III of FIG. 1, found on VGAM181 host target RNA, and schematic representation of the complementarity of each of these host target binding sites to VGAM181 RNA, herein designated VGAM RNA, are described hereinbelow with reference to Table 2.

As mentioned hereinabove with reference to FIG. 1, a function of VGAM181 gene, herein designated VGAM is inhibition of expression of VGAM181 target genes. It is appreciated that specific functions, and accordingly utilities, of VGAM181 correlate with, and may be deduced from, the identity of the target genes which VGAM181 binds and inhibits, and the function of these target genes, as elaborated hereinbelow.

BTG Family, Member 2 (BTG2, Accession NM_{—}006763) is a VGAM181 host target gene. BTG2 BINDING SITE is HOST TARGET binding site found in the 3′ untranslated region of mRNA encoded by BTG2, corresponding to a HOST TARGET binding site such as BINDING SITE I, BINDING SITE II or BINDING SITE III. Table 2 illustrates the complementarity of the nucleotide sequences of BTG2 BINDING SITE, designated SEQ ID:1330, to the nucleotide sequence of VGAM181 RNA, herein designated VGAM RNA, also designated SEQ ID:516.

A function of VGAM181 is therefore inhibition of BTG Family, Member 2 (BTG2, Accession NM_{—}006763). Accordingly, utilities of VGAM181 include diagnosis, prevention and treatment of diseases and clinical conditions associated with BTG2. Cadherin, EGF LAG Seven-pass G-type Receptor 2 (flamingo homolog, Drosophila) (CELSR2, Accession NM_{—}001408) is another VGAM181 host target gene. CELSR2 BINDING SITE is HOST TARGET binding site found in the 3′ untranslated region of mRNA encoded by CELSR2, corresponding to a HOST TARGET binding site such as BINDING SITE I, BINDING SITE II or BINDING SITE III. Table 2 illustrates the complementarity of the nucleotide sequences of CELSR2 BINDING SITE, designated SEQ ID:826, to the nucleotide sequence of VGAM181 RNA, herein designated VGAM RNA, also designated SEQ ID:516.

Another function of VGAM181 is therefore inhibition of Cadherin, EGF LAG Seven-pass G-type Receptor 2 (flamingo homolog, Drosophila) (CELSR2, Accession NM_{—}001408), a gene which is a calcium dependent cell adhesion protein. Accordingly, utilities of VGAM181 include diagnosis, prevention and treatment of diseases and clinical conditions associated with CELSR2. The function of CELSR2 has been established by previous studies. The domain that characterizes epidermal growth factor (EGF; 131530) consists of approximately 50 amino acids with 3 disulfide bonds. EGF-like domains are believed to play a critical role in a number of extracellular events, including cell adhesion and receptor-ligand interactions. Proteins with EGF-like domains often consist of more than 1,000 amino acids, have multiple copies of the EGF-like domain, and contain additional domains known to be involved in specific protein-protein interactions. To identify proteins containing EGF-like domains, Nakayama et al. (1998) searched a database of long cDNA sequences randomly selected from a human brain cDNA library for those that encode an EGF-like motif. They identified several partial cDNAs encoding novel proteins with EGF-like domains, such as EGFL2, which they named MEGF3. The predicted partial EGFL2 protein has at least 5 cadherin motifs, 6 EGF-like domains, 2 laminin G domains (see OMIM Ref. No. 601033), 7 transmembrane domains, and a cytoplasmic proline-rich sequence. Human EGFL2 appears to have a domain structure identical to that of rat Megf2 (OMIM Ref. No. 604264), whose complete coding sequence was also isolated by the authors. Northern blot analysis detected rat Megf3 expression in several regions of the brain.

Full details of the abovementioned studies are described in the following publications, the disclosure of which are hereby incorporated by reference:
Nagase, T.; Seki, N.; Ishikawa, K.; Ohira, M.; Kawarabayasi, Y.; Ohara, O.; Tanaka, A.; Kotani, H.; Miyajima, N.; Nomura, N.: Prediction of the coding sequences of unidentified human genes. VI. The coding sequences of 80 new genes (KIAA0201-KIAA0280) deduced by analysis of cDNA clones from cell line KG-1 and brain. DNA Res. 3: 321-329, 1996.; and
Nakayama, M.; Nakajima, D.; Nagase, T.; Nomura, N.; Seki, N.; Ohara, O.: Identification of high-molecular-weight proteins with multiple EGF-like motifs by motif-trap screening. Genomic.

Further studies establishing the function and utilities of CELSR2 are found in John Hopkins OMIM database record ID 604265, and in sited publications numbered 2121 listed in the bibliography section hereinbelow, which are also hereby incorporated by reference. GRB2-associated Binding Protein 2 (GAB2, Accession NM_{—}012296) is another VGAM181 host target gene. GAB2 BINDING SITE1 and GAB2 BINDING SITE2 are HOST TARGET binding sites found in untranslated regions of mRNA encoded by GAB2, corresponding to HOST TARGET binding sites such as BINDING SITE I, BINDING SITE II or BINDING SITE III. Table 2 illustrates the complementarity of the nucleotide sequences of GAB2 BINDING SITE1 and GAB2 BINDING SITE2, designated SEQ ID: 1424 and SEQ ID:2375 respectively, to the nucleotide sequence of VGAM181 RNA, herein designated VGAM RNA, also designated SEQ ID:516.

Another function of VGAM181 is therefore inhibition of GRB2-associated Binding Protein 2 (GAB2, Accession NM_{—}012296), a gene which act as adapters for transmitting various signals. Accordingly, utilities of VGAM181 include diagnosis, prevention and treatment of diseases and clinical conditions associated with GAB2. The function of GAB2 has been established by previous studies. The GAB2 gene encodes a 100-kd adapter molecule that is the principal activator of phosphatidylinositol-3 kinase (PIK3; OMIM Ref. No. 171833) in response to activation of the high affinity IgE receptor (see OMIM Ref. No. 147140). Zhao et al. (1999) demonstrated that upon tyrosine phosphorylation, GAB2 physically interacts with SHP2 tyrosine phosphatase and GRB2 adapter protein (OMIM Ref. No. 604330). GAB2 has an inhibitory effect on the activation of ELK1 (OMIM Ref. No. 311040)-dependent transcription triggered by a dominant active Ras (OMIM Ref. No. 190020) mutant or under growth factor stimulation, whereas GAB1 acts to potentiate slightly the ELK1 activity in the same system. In contrast to the reciprocal effects of GAB1 and GAB2 in mediating ELK1 induction, these 2 molecules have a similar function in extracellular signal-regulated kinase activation induced by either oncogenic Ras or growth factor stimulation. Zhao et al. (1999) concluded that GAB1 and GAB2 may have distinct roles in coupling cytoplasmic-nuclear signal transduction. Animal model experiments lend further support to the function of GAB2. Gu et al. (2001) generated mice deficient in Gab2 by homologous recombination. Gab2 −/− mice were viable and generally healthy; however, the response of Gab2 −/− mast cells to stimulation of the high affinity IgE receptor Fc-epsilon-R1 (see OMIM Ref. No. 147140) was defective. Accordingly, allergic reactions, such as passive cutaneous and systemic anaphylaxis, were markedly impaired in Gab −/− mice. Biochemical analyses revealed that signaling pathways dependent on phosphatidylinositol-3 hydroxykinase (PI3K), a critical component of the Fc-epsilon-R1 signaling, were defective in Gab2 −/− mast cells. Gu et al. (2001) concluded that GAB2 is the principal activator of PI3K in response to Fc-epsilon-R1 activation, thereby providing genetic evidence that Dos/Gab family scaffolds regulate the PI3K pathway in vivo.

It is appreciated that the abovementioned animal model for GAB2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Gu, H.; Saito, K.; Klaman, L. D.; Shen, J.; Fleming, T.; Wang, Y.-P.; Pratt, J. C.; Lin, G.; Lim, B.; Kinet, J.-P.; Neel, B. G.: Essential role for Gab2 in the allergic response. Nature 412: 186-190, 2001.; and
Zhao, C.; Yu, D.-H.; Shen, R.; Feng, G.-S.: Gab2, a new pleckstrin homology domain-containing adapter protein, acts to uncouple signaling from ERK kinase to Elk-1. J. Biol. Chem. 274.

Further studies establishing the function and utilities of GAB2 are found in John Hopkins OMIM database record ID 606203, and in sited publications numbered 212, 156 and 1813 listed in the bibliography section hereinbelow, which are also hereby incorporated by reference. MADS Box Transcription Enhancer Factor 2, Polypeptide D (myocyte enhancer factor 2D) (MEF2D, Accession XM_{—}173049) is another VGAM181 host target gene. MEF2D BINDING SITE is HOST TARGET binding site found in the 3′ untranslated region of mRNA encoded by MEF2D, corresponding to a HOST TARGET binding site such as BINDING SITE I, BINDING SITE II or BINDING SITE III. Table 2 illustrates the complementarity of the nucleotide sequences of MEF2D BINDING SITE, designated SEQ ID:3721, to the nucleotide sequence of VGAM181 RNA, herein designated VGAM RNA, also designated SEQ ID:516.

Another function of VGAM181 is therefore inhibition of MADS Box Transcription Enhancer Factor 2, Polypeptide D (myocyte enhancer factor 2D) (MEF2D, Accession XM_{—}173049), a gene which regulates muscle-specific and mitogen-inducible genes. Accordingly, utilities of VGAM181 include diagnosis, prevention and treatment of diseases and clinical conditions associated with MEF2D. The function of MEF2D has been established by previous studies. The MEF2 family of regulatory proteins are, like the myogenic basic helix-loop-helix proteins (see OMIM Ref. No. 159970), involved in myogenesis; see (OMIM Ref. No. 600660). Breitbart et al. (1993) obtained MEF2D cDNAs from an adult cardiac ventricle expression library screened at low stringency with a human MEF2B probe. One of the cDNAs encoded a 521-amino acid protein with highly conserved MADS and MEF2 domains. The recombinantly expressed MEF2D protein showed DNA binding to the MEF2 site. Breitbart et al. (1993) found that MEF2D occurs as several alternatively spliced transcripts, one of which resembles the Xenopus SRF-related factor SL-1. Unlike the other MEF2 family members, MEF2D is present in undifferentiated myoblasts and may participate in the earliest stages of commitment. Hobson et al. (1995) mapped the MEF2D gene to 1q12-q23 using somatic cell hybrid panel DNAs containing deletion or derivative chromosomes. Mouse Mef2D was mapped by Martin et al. (1994) to chromosome 3. Ikeshima et al. (1995) demonstrated strong expression of MEF2D in the cerebellum and cerebrum of developing mouse brains and also in central nervous system neurons of adult mice, suggesting that it may be involved in the differentiation of neurogenic as well as myogenic cells.

Full details of the abovementioned studies are described in the following publications, the disclosure of which are hereby incorporated by reference:
Hobson, G. M.; Krahe, R.; Garcia, E.; Siciliano, M. J.; Funanage, V. L.: Regional chromosomal assignments for four members of the MADS domain transcription enhancer factor 2 (MEF2) gene family to human chromosomes 15q26, 19p12, 5q14, and 1q12-q23. Genomics 29: 704-711, 1995.; and
Ikeshima, H.; Imai, S.; Shimoda, K.; Hata, J.; Takano, T.: Expression of a MADS box gene, MEF2D, in neurons of the mouse central nervous system: implication of its binary function in m.

Further studies establishing the function and utilities of MEF2D are found in John Hopkins OMIM database record ID 600663, and in sited publications numbered 1888-1889, 186 and 1891 listed in the bibliography section hereinbelow, which are also hereby incorporated by reference. NEBL (Accession NM_{—}006393) is another VGAM181 host target gene. NEBL BINDING SITE is HOST TARGET binding site found in the 5′ untranslated region of mRNA encoded by NEBL, corresponding to a HOST TARGET binding site such as BINDING SITE I, BINDING SITE II or BINDING SITE III. Table 2 illustrates the complementarity of the nucleotide sequences of NEBL BINDING SITE, designated SEQ ID:1294, to the nucleotide sequence of VGAM181 RNA, herein designated VGAM RNA, also designated SEQ ID:516.

Another function of VGAM181 is therefore inhibition of NEBL (Accession NM_{—}006393). Accordingly, utilities of VGAM181 include diagnosis, prevention and treatment of diseases and clinical conditions associated with NEBL. Proprotein Convertase Subtilisin/kexin Type 1 (PCSK1, Accession NM_{—}000439) is another VGAM181 host target gene. PCSK1 BINDING SITE is HOST TARGET binding site found in the 3′ untranslated region of mRNA encoded by PCSK1, corresponding to a HOST TARGET binding site such as BINDING SITE I, BINDING SITE II or BINDING SITE III. Table 2 illustrates the complementarity of the nucleotide sequences of PCSK1 BINDING SITE, designated SEQ ID:743, to the nucleotide sequence of VGAM181 RNA, herein designated VGAM RNA, also designated SEQ ID:516.

Another function of VGAM181 is therefore inhibition of Proprotein Convertase Subtilisin/kexin Type 1 (PCSK1, Accession NM_{—}000439), a gene which processes hormone precursors by cleaving paired basic amino acids; serine protease of the subtilase family. Accordingly, utilities of VGAM181 include diagnosis, prevention and treatment of diseases and clinical conditions associated with PCSK1. The function of PCSK1 has been established by previous studies. A wide variety of biologically important polypeptides including hormones, enzymes, and receptors are initially synthesized as large inactive precursors. To release the active component(s), these precursors must undergo limited proteolysis at pairs of basic residues by specific convertases. There is, for example, a diarginyl-specific proalbumin convertase (see OMIM Ref. No. comment in 103600). Three mammalian convertases, PC1 (PCSK1; also known as PC3), PC2 (PCSK2; 162151), and furin (OMIM Ref. No. 136950), belonging to the family of serine proteinases of the subtilisin family, are prohormone and proprotein convertases. PC1 and PC2, known also as NEC1 and NEC2 (for neuroendocrine convertase 1 and 2, respectively), differentially cleave proopiomelanocortin (POMC; 176830). Proinsulin is converted to insulin (OMIM Ref. No. 176730) by the concerted action of PC2 and PC3. Furin is a specific proteinase capable of activating the beta subunit of pro-NGF (OMIM Ref. No. 162030) and von Willebrand factor (OMIM Ref. No. 193400). By in situ hybridization, Seidah et al. (1991) mapped NEC1 to human 5q15-q21 and to mouse chromosome 13. Copeland et al. (1992) refined the regional localization on mouse chromosome 13. Ohagi et al. (1996) stated that PC2 is responsible for cleavage of the C-peptide/A-chain junction of the proinsulin molecule, whereas PC3 cleaves the proinsulin molecule on the C-terminal side of the dibasic peptide, arg31-arg32, joining the B-chain and C-peptide. PC3 plays a key role in regulating insulin biosynthesis by initiating the sequential processing. Expression of insulin and PC3, but not PC2, is coordinately regulated by glucose, consistent with the important role of PC3 in regulating proinsulin processing. Noninsulin-dependent diabetes mellitus (NIDDM; 125853) is associated with increased secretion of proinsulin and proinsulin-like molecules, suggesting that mutations in the PC3 gene may be involved in the development of this disorder. Ohagi et al. (1996) showed that the human PC3 gene consists of 14 exons spanning more than 35 kb. The exon/intron organization of the PC2 and PC3 genes are conserved, consistent with a common evolutionary origin. Screening for mutations in the PC3 gene in Japanese subjects with NIDDM using SSCP analysis and nucleotide sequencing of the entire coding region, Ohagi et al. (1996) could find no mutation associated with NIDDM. A mutation in carboxypeptidase E (CPE; 114855), an enzyme active in the processing and sorting of prohormones, causes obesity in the fat/fat mouse (Naggert et al., 1995; Cool et al., 1997). The gene products of CPE and PC1 cooperate in prohormone processing. Mutations in the CPE gene had not been demonstrated in human obesity. However, Jackson et al. (1997) demonstrated mutations in the prohormone convertase 1 gene, which acts proximally to CPE in the pathway of posttranslational processing of prohormones and neuropeptides. The subject was a 43-year-old woman with extreme childhood obesity, abnormal glucose homeostasis, hypogonadotropic hypogonadism, hypocortisolism, and elevated plasma proinsulin and POMC concentrations, but very low insulin levels, all suggestive of defective prohormone processing by the patient's PC1. The patient had been described clinically by O'Rahilly et al. (1995); see 600955. The patient was found to be a compound heterozygote for mutations in PC1. Heteroallelism of the patient was confirmed by the fact that 1 substitution (162150.0001) was found in 3 of the proband's 4 children, all of whom were clinically unaffected; the fourth child had the other mutation, a splice site defect (162150.0002). The proband's fasting serum leptin (OMIM Ref. No. 164160) concentration was appropriate for her body mass index. There was a close similarity of phenotype between the proband and the fat/fat mouse.

Full details of the abovementioned studies are described in the following publications, the disclosure of which are hereby incorporated by reference:
Ohagi, S.; Sakaguchi, H.; Sanke, T.; Tatsuta, H.; Hanabusa, T.; Nanjo, K.: Human prohormone convertase 3 gene: exon-intron organization and molecular scanning for mutations in Japanese subjects with NIDDM. Diabetes 45: 897-901, 1996.; and
O'Rahilly, S.; Gray, H.; Humphreys, P. J.; Krook, A.; Polonsky, K. S.; White, A.; Gibson, S.; Taylor, K.; Carr, C.: Brief report: impaired processing of prohormones associated with abno.

Further studies establishing the function and utilities of PCSK1 are found in John Hopkins OMIM database record ID 162150, and in sited publications numbered 362, 844-41 and 846 listed in the bibliography section hereinbelow, which are also hereby incorporated by reference. Protein Kinase, CAMP-dependent, Regulatory, Type II, Beta (PRKAR2B, Accession NM_{—}002736) is another VGAM181 host target gene. PRKAR2B BINDING SITE is HOST TARGET binding site found in the 3′ untranslated region of mRNA encoded by PRKAR2B, corresponding to a HOST TARGET binding site such as BINDING SITE I, BINDING SITE II or BINDING SITE III. Table 2 illustrates the complementarity of the nucleotide sequences of PRKAR2B BINDING SITE, designated SEQ ID:951, to the nucleotide sequence of VGAM181 RNA, herein designated VGAM RNA, also designated SEQ ID:516.

Another function of VGAM181 is therefore inhibition of Protein Kinase, CAMP-dependent, Regulatory, Type II, Beta (PRKAR2B, Accession NM_{—}002736), a gene which type ii regulatory chains mediate membrane association by binding to anchoring proteins, including the map2 kinase. Accordingly, utilities of VGAM181 include diagnosis, prevention and treatment of diseases and clinical conditions associated with PRKAR2B. The function of PRKAR2B has been established by previous studies. Using both a rat skeletal muscle clone and a human clone of type II regulatory subunit of cyclic AMP-dependent protein kinase, Scambler et al. (1987) demonstrated that the human gene is located on chromosome 7, close to but separate from the cystic fibrosis locus (OMIM Ref. No. 219700). These conclusions were based on Southern blot analysis of DNA from hybrid cell lines containing only chromosome 7 or parts thereof, as well as human/mouse hybrid cell lines established by means of chromosome-mediated gene transfer (CMGT) using MET (OMIM Ref. No. 164860) as a dominant selectable marker. Independence of PKR2 from CF was also indicated by family linkage studies using a RFLP of the PKR2 probe. Wainwright et al. (1987) showed that PKR2 is linked to several markers on 7q. The closest and strongest linkage was to TCRB (OMIM Ref. No. 186930), which showed a maximum Iod score of 3.01 at theta=0.00. Using RFLPs in the CEPH panel of 40 families, Solberg et al. (1992) mapped the regulatory subunit RII-beta of cAMP-dependent protein kinase to 7q. They constructed a 7-point framework map including PRKAR2B and demonstrated the following order: cen—D7S371—(COL1A2, D7S79)—PRKAR2 B—MET—D7S87—TCRB—qter. Furthermore, by in situ hybridization to metaphase chromosomes, Solberg et al. (1992) physically mapped PRKAR2B to 7q22. Cummings et al. (1996) generated knockout mice for the cyclic AMP dependent protein kinase regulatory subunit type II-beta (designated RII-beta by them). They reported that the mutants appeared healthy but had markedly diminished white adipose tissue despite normal food intake and were protected against developing diet-induced obesity and fatty livers. In the mutant mice, brown adipose tissue demonstrated a compensatory increase in R1-alpha (OMIM Ref. No. 188830). Cummings et al. (1996) reported that RII-beta mutants exhibited markedly reduced leptin (OMIM Ref. No. 164160) mRNA and plasma levels; however, only mild hyperphagia was present

Full details of the abovementioned studies are described in the following publications, the disclosure of which are hereby incorporated by reference:
Cummings, D. E.; Brandon, E. P.; Planas, J. V.; Motamed, K.; Idzerda, R. L.; McKnight, G. S.: Genetically lean mice result from targeted disruption of the RII-beta subunit of protein kinase A. Nature 382: :622-626, 1996.; and
Scambler, P.; Oyen, O.; Wainwright, B.; Farrall, M.; Law, H.-Y.; Estivill, X.; Sandberg, M.; Williamson, R.; Jahnsen, T.: Exclusion of catalytic and regulatory subunits of cAMP-dependent.

Further studies establishing the function and utilities of PRKAR2B are found in John Hopkins OMIM database record ID 176912, and in sited publications numbered 282-283, 28 and 284 listed in the bibliography section hereinbelow, which are also hereby incorporated by reference. Secretoglobin, Family 3A, Member 2 (SCGB3A2, Accession NM_{—}054023) is another VGAM181 host target gene. SCGB3A2 BINDING SITE is HOST TARGET binding site found in the 3′ untranslated region of mRNA encoded by SCGB3A2, corresponding to a HOST TARGET binding site such as BINDING SITE I, BINDING SITE II or BINDING SITE III. Table 2 illustrates the complementarity of the nucleotide sequences of SCGB3A2 BINDING SITE, designated SEQ ID:2361, to the nucleotide sequence of VGAM181 RNA, herein designated VGAM RNA, also designated SEQ ID:516.

Another function of VGAM181 is therefore inhibition of Secretoglobin, Family 3A, Member 2 (SCGB3A2, Accession NM_{—}054023), a gene which is a uteroglobin-related protein and is a downstream target of TITF1. Accordingly, utilities of VGAM181 include diagnosis, prevention and treatment of diseases and clinical conditions associated with SCGB3A2. The function of SCGB3A2 has been established by previous studies. In addition to regulating thyroid-specific expression of genes, thyroid transcription factor (TITF1; 600635) controls the transcription of genes specifically expressed in lung, such as surfactant proteins (e.g., SFTPA1; 178630) and uteroglobin (UGB; 192020). Mice lacking Ttf1 die immediately after birth from respiratory failure caused by profoundly hypoplastic lungs (Kimura et al., 1996). The UGRP1 gene encodes a uteroglobin-related protein and is a downstream target of TITF1. Niimi et al. (2002) presented evidence that UGRP1 is one of the asthma-susceptibility genes located on 5q31-q32. This homodimeric secretory protein expressed only in lung and trachea was found to have a G-to-A polymorphism at −112 bp in the gene promoter region. The −112A allele is responsible for a 24% reduction in the promoter activity in relation to the −112G allele, as examined by transfection analysis. Electrophoretic mobility-shift analysis revealed that an unknown nuclear factor binds to the region around −112 bp. The binding affinity with the −112A oligonucleotide was reduced by approximately one-half, as compared with the −112G oligonucleotide. In a case-control study using 169 Japanese individuals (84 patients with asthma and 85 health control individuals), those with the −112A allele (G/A or A/A) were 4.1 times more likely to have asthma than were those with the wildtype allele (G/G).

Full details of the abovementioned studies are described in the following publications, the disclosure of which are hereby incorporated by reference:
Niimi, T.; Munakata, M.; Keck-Waggoner, C. L.; Popescu, N. C.; Levitt, R. C.; Hisada, M.; Kimura, S.: A polymorphism in the human UGRP1 gene promoter that regulates transcription is associated with an increased risk of asthma. Am. J. Hum. Genet. 70: 718-725, 2002.; and
Niimi, T.; Keck-Waggoner, C. L.; Popescu, N. C.; Zhou, Y.; Levitt, R. C.; Kimura, S.: UGRP1, a uteroglobin/Clara cell secretory protein-related protein, is a novel lung-enriched downst.

Further studies establishing the function and utilities of SCGB3A2 are found in John Hopkins OMIM database record ID 606531, and in sited publications numbered 1627-177 listed in the bibliography section hereinbelow, which are also hereby incorporated by reference. SMP1 (Accession NM_{—}014313) is another VGAM181 host target gene. SMP1 BINDING SITE is HOST TARGET binding site found in the 3′ untranslated region of mRNA encoded by SMP1, corresponding to a HOST TARGET binding site such as BINDING SITE I, BINDING SITE II or BINDING SITE III. Table 2 illustrates the complementarity of the nucleotide sequences of SMP1 BINDING SITE, designated SEQ ID: 1490, to the nucleotide sequence of VGAM181 RNA, herein designated VGAM RNA, also designated SEQ ID:516.

Another function of VGAM181 is therefore inhibition of SMP1 (Accession NM_{—}014313), a gene which is a potential integral membrane protein. Accordingly, utilities of VGAM181 include diagnosis, prevention and treatment of diseases and clinical conditions associated with SMP1. The function of SMP1 has been established by previous studies. Wagner and Flegel (2000) found that the RH cluster on chromosome 1p contains 3 genes: RHD (OMIM Ref. No. 111680), RHCE (OMIM Ref. No. 111700), and SMP1. They noted that the nucleotide sequence of SMP1 had been deposited in GenBank (AF091282) as encoding a putative 157-amino acid member of an 18-kD small membrane protein family and that the gene shows homology to an open reading frame on chromosome 21 (Reboul et al., 1999). The position of the gene between both RH genes implies that any polymorphism of the SMP1 gene would be tightly linked to a specific RH haplotype. The authors suggested that functionally relevant mutations of the SMP1 gene may cause selection pressure for or against specific RH haplotypes. Such factors might explain some previously unresolved issues of RH haplotype distribution, such as the high frequency of RH-negativity in the European population.

Full details of the abovementioned studies are described in the following publications, the disclosure of which are hereby incorporated by reference:
Reboul, J.; Gardiner, K.; Monneron, D.; Uze, G.; Lutfalla, G.: Comparative genomic analysis of the interferon/interleukin-10 receptor gene cluster. Genome Res. 9: 242-250, 1999.; and
Wagner, F. F.; Flegel, W. A.: RHD gene deletion occurred in the Rhesus box. Blood 95: 3662-3668, 2000.

Further studies establishing the function and utilities of SMP1 are found in John Hopkins OMIM database record ID 605348, and in sited publications numbered 1440 listed in the bibliography section hereinbelow, which are also hereby incorporated by reference. Synaptogyrin 1 (SYNGR1, Accession NM_{—}004711) is another VGAM181 host target gene. SYNGR1 BINDING SITE is HOST TARGET binding site found in the 3′ untranslated region of mRNA encoded by SYNGR1, corresponding to a HOST TARGET binding site such as BINDING SITE I, BINDING SITE II or BINDING SITE III. Table 2 illustrates the complementarity of the nucleotide sequences of SYNGR1 BINDING SITE, designated SEQ ID:1147, to the nucleotide sequence of VGAM181 RNA, herein designated VGAM RNA, also designated SEQ ID:516.

Another function of VGAM181 is therefore inhibition of Synaptogyrin 1 (SYNGR1, Accession NM_{—}004711), a gene which belongs to transmembrane synaptic vesicle protein and may function in membrane recycling. Accordingly, utilities of VGAM181 include diagnosis, prevention and treatment of diseases and clinical conditions associated with SYNGR1. The function of SYNGR1 and its association with various diseases and clinical conditions, has been established by previous studies, as described hereinabove with reference to VGAM97. Transforming Growth Factor, Alpha (TGFA, Accession NM_{—}003236) is another VGAM181 host target gene. TGFA BINDING SITE is HOST TARGET binding site found in the 3′ untranslated region of mRNA encoded by TGFA, corresponding to a HOST TARGET binding site such as BINDING SITE I, BINDING SITE II or BINDING SITE III. Table 2 illustrates the complementarity of the nucleotide sequences of TGFA BINDING SITE, designated SEQ ID: 1001, to the nucleotide sequence of VGAM181 RNA, herein designated VGAM RNA, also designated SEQ ID:516.

Another function of VGAM181 is therefore inhibition of Transforming Growth Factor, Alpha (TGFA, Accession NM_{—}003236), a gene which is able to bind to the egf receptor and to act synergistically with tgf beta to promote anchorage-independent cell proliferation in soft agar. Accordingly, utilities of VGAM181 include diagnosis, prevention and treatment of diseases and clinical conditions associated with TGFA. The function of TGFA has been established by previous studies. Ellis et al. (1987) presented evidence that TGFA plays a role in certain paraneoplastic manifestations of melanoma: the sign of Leser-Trelat (the sudden appearance of, or increase in the number and size of, seborrheic keratoses), acanthosis nigricans, and eruptive acrochordons (sudden onset of multiple skin tags). Fernandez-Larrea et al. (1999) used the 2-hybrid screen to identify pro-TGF-alpha cytoplasmic domain-binding proteins, which they referred to as TACIPs (pro-TGF-alpha cytoplasmic domain-interacting proteins), involved in the trafficking of pro-TGF-alpha. They cloned 2 such proteins, which they designated TACIP1 (OMIM Ref. No. 601017) and TACIP18 (OMIM Ref. No. 602217). The circadian clock in the suprachiasmatic nucleus is thought to drive daily rhythms of behavior by secreting factors that act locally within the hypothalamus. In a systematic screen, Kramer et al. (2001) identified TGFA as a likely suprachiasmatic nucleus inhibitor of locomotion. TGFA is expressed rhythmically in the suprachiasmatic nucleus, and when infused into the third ventricle it reversibly inhibited locomotor activity and disrupted circadian sleep-wake cycles. These actions were mediated by EGF receptors on neurons in the hypothalamic subparaventricular zone. Mice with a hypomorphic EGF receptor mutation exhibited excessive daytime locomotor activity and failed to suppress activity when exposed to light. Kramer et al. (2001) concluded that their results implicate EGF receptor signaling in the daily control of locomotor activity. They identified a neural circuit in the hypothalamus that likely mediates the regulation of behavior both by the suprachiasmatic nucleus and the retina using TGFA and EGF receptors in the retinohypothalamic tract.

Full details of the abovementioned studies are described in the following publications, the disclosure of which are hereby incorporated by reference:
Fernandez-Larrea, J.; Merlos-Suarez, A.; Urena, J. M.; Baselga, J.; Arribas, J.: A role for a PDZ protein in the early secretory pathway for the targeting of proTGF-alpha to the cell surface. Molec. Cell 3: 423-433, 1999.; and
Kramer, A.; Yang, F.-C.; Snodgrass, P.; Li, X.; Scammell, T. E.; Davis, F. C.; Weitz, C. J.: Regulation of daily locomotor activity and sleep by hypothalamic EGF receptor signaling. S.

Further studies establishing the function and utilities of TGFA are found in John Hopkins OMIM database record ID 190170, and in sited publications numbered 616-620, 7 and 621-623 listed in the bibliography section hereinbelow, which are also hereby incorporated by reference. Acetyl-Coenzyme A Acyltransferase 2 (mitochondrial 3-oxoacyl-Coenzyme A thiolase) (ACAA2, Accession XM_{—}166287) is another VGAM181 host target gene. ACAA2 BINDING SITE is HOST TARGET binding site found in the 5′ untranslated region of mRNA encoded by ACAA2, corresponding to a HOST TARGET binding site such as BINDING SITE I, BINDING SITE II or BINDING SITE III. Table 2 illustrates the complementarity of the nucleotide sequences of ACAA2 BINDING SITE, designated SEQ ID:3540, to the nucleotide sequence of VGAM181 RNA, herein designated VGAM RNA, also designated SEQ ID:516.

Another function of VGAM181 is therefore inhibition of Acetyl-Coenzyme A Acyltransferase 2 (mitochondrial 3-oxoacyl-Coenzyme A thiolase) (ACAA2, Accession XM_{—}166287). Accordingly, utilities of VGAM181 include diagnosis, prevention and treatment of diseases and clinical conditions associated with ACAA2. Chromosome 1 Open Reading Frame 34 (Clorf34, Accession XM_{—}027172) is another VGAM181 host target gene. Clorf34 BINDING SITE is HOST TARGET binding site found in the 3′ untranslated region of mRNA encoded by Clorf34, corresponding to a HOST TARGET binding site such as BINDING SITE I, BINDING SITE II or BINDING SITE III. Table 2 illustrates the complementarity of the nucleotide sequences of Clorf34 BINDING SITE, designated SEQ ID:2576, to the nucleotide sequence of VGAM181 RNA, herein designated VGAM RNA, also designated SEQ ID:516.

Another function of VGAM181 is therefore inhibition of Chromosome 1 Open Reading Frame 34 (Clorf34, Accession XM_{—}027172). Accordingly, utilities of VGAM181 include diagnosis, prevention and treatment of diseases and clinical conditions associated with Clorf34. Calcium/calmodulin-dependent Protein Kinase Kinase 2, Beta (CAMKK2, Accession NM_{—}006549) is another VGAM181 host target gene. CAMKK2 BINDING SITE is HOST TARGET binding site found in the 3′ untranslated region of mRNA encoded by CAMKK2, corresponding to a HOST TARGET binding site such as BINDING SITE I, BINDING SITE II or BINDING SITE III. Table 2 illustrates the complementarity of the nucleotide sequences of CAMKK2 BINDING SITE, designated SEQ ID:1310, to the nucleotide sequence of VGAM181 RNA, herein designated VGAM RNA, also designated SEQ ID:516.

Another function of VGAM181 is therefore inhibition of Calcium/calmodulin-dependent Protein Kinase Kinase 2, Beta (CAMKK2, Accession NM_{—}006549). Accordingly, utilities of VGAM181 include diagnosis, prevention and treatment of diseases and clinical conditions associated with CAMKK2. FLJ10849 (Accession NM_{—}018243) is another VGAM181 host target gene. FLJ10849 BINDING SITE is HOST TARGET binding site found in the 3′ untranslated region of mRNA encoded by FLJ10849, corresponding to a HOST TARGET binding site such as BINDING SITE I, BINDING SITE II or BINDING SITE III. Table 2 illustrates the complementarity of the nucleotide sequences of FLJ10849 BINDING SITE, designated SEQ ID:1803, to the nucleotide sequence of VGAM181 RNA, herein designated VGAM RNA, also designated SEQ ID:516.

Another function of VGAM181 is therefore inhibition of FLJ10849 (Accession NM_{—}018243). Accordingly, utilities of VGAM181 include diagnosis, prevention and treatment of diseases and clinical conditions associated with FLJ10849. FLJ13262 (Accession NM_{—}024914) is another VGAM181 host target gene. FLJ13262 BINDING SITE is HOST TARGET binding site found in the 3′ untranslated region of mRNA encoded by FLJ13262, corresponding to a HOST TARGET binding site such as BINDING SITE I, BINDING SITE II or BINDING SITE III. Table 2 illustrates the complementarity of the nucleotide sequences of FLJ13262 BINDING SITE, designated SEQ ID:2112, to the nucleotide sequence of VGAM181 RNA, herein designated VGAM RNA, also designated SEQ ID:516.

Another function of VGAM181 is therefore inhibition of FLJ13262 (Accession NM_{—}024914). Accordingly, utilities of VGAM181 include diagnosis, prevention and treatment of diseases and clinical conditions associated with FLJ13262. FLJ21977 (Accession NM_{—}032213) is another VGAM181 host target gene. FLJ21977 BINDING SITE is HOST TARGET binding site found in the 5′ untranslated region of mRNA encoded by FLJ21977, corresponding to a HOST TARGET binding site such as BINDING SITE I, BINDING SITE II or BINDING SITE III. Table 2 illustrates the complementarity of the nucleotide sequences of FLJ21977 BINDING SITE, designated SEQ ID:2237, to the nucleotide sequence of VGAM181 RNA, herein designated VGAM RNA, also designated SEQ ID:516.

Another function of VGAM181 is therefore inhibition of FLJ21977 (Accession NM_{—}032213). Accordingly, utilities of VGAM181 include diagnosis, prevention and treatment of diseases and clinical conditions associated with FLJ21977. FLJ22301 (Accession NM_{—}024836) is another VGAM181 host target gene. FLJ22301 BINDING SITE is HOST TARGET binding site found in the 3′ untranslated region of mRNA encoded by FLJ22301, corresponding to a HOST TARGET binding site such as BINDING SITE I, BINDING SITE II or BINDING SITE III. Table 2 illustrates the complementarity of the nucleotide sequences of FLJ22301 BINDING SITE, designated SEQ ID:2095, to the nucleotide sequence of VGAM181 RNA, herein designated VGAM RNA, also designated SEQ ID:516.

Another function of VGAM181 is therefore inhibition of FLJ22301 (Accession NM_{—}024836). Accordingly, utilities of VGAM181 include diagnosis, prevention and treatment of diseases and clinical conditions associated with FLJ22301. Hect (homologous to the E6-AP (UBE3A) Carboxyl Terminus) Domain and RCC1 (CHC1)-like Domain (RLD) 1 (HERC1, Accession NM_{—}003922) is another VGAM181 host target gene. HERC1 BINDING SITE is HOST TARGET binding site found in the 3′ untranslated region of mRNA encoded by HERC1, corresponding to a HOST TARGET binding site such as BINDING SITE I, BINDING SITE II or BINDING SITE III. Table 2 illustrates the complementarity of the nucleotide sequences of HERC1 BINDING SITE, designated SEQ ID:1071, to the nucleotide sequence of VGAM181 RNA, herein designated VGAM RNA, also designated SEQ ID:516.

Another function of VGAM181 is therefore inhibition of Hect (homologous to the E6-AP (UBE3A) Carboxyl Terminus) Domain and RCC1 (CHC1)-like Domain (RLD) 1 (HERC1, Accession NM_{—}003922). Accordingly, utilities of VGAM181 include diagnosis, prevention and treatment of diseases and clinical conditions associated with HERC1. KIAA0280 (Accession XM_{—}166238) is another VGAM181 host target gene. KIAA0280 BINDING SITE is HOST TARGET binding site found in the 3′ untranslated region of mRNA encoded by KIAA0280, corresponding to a HOST TARGET binding site such as BINDING SITE I, BINDING SITE II or BINDING SITE III. Table 2 illustrates the complementarity of the nucleotide sequences of KIAA0280 BINDING SITE, designated SEQ ID:3536, to the nucleotide sequence of VGAM181 RNA, herein designated VGAM RNA, also designated SEQ ID:516.

Another function of VGAM181 is therefore inhibition of KIAA0280 (Accession XM_{—}166238). Accordingly, utilities of VGAM181 include diagnosis, prevention and treatment of diseases and clinical conditions associated with KIAA0280. KIAA0391 (Accession NM_{—}014672) is another VGAM181 host target gene. KIAA0391 BINDING SITE is HOST TARGET binding site found in the 3′ untranslated region of mRNA encoded by KIAA0391, corresponding to a HOST TARGET binding site such as BINDING SITE I, BINDING SITE II or BINDING SITE III. Table 2 illustrates the complementarity of the nucleotide sequences of KIAA0391 BINDING SITE, designated SEQ ID:1517, to the nucleotide sequence of VGAM181 RNA, herein designated VGAM RNA, also designated SEQ ID:516.

Another function of VGAM181 is therefore inhibition of KIAA0391 (Accession NM_{—}014672). Accordingly, utilities of VGAM181 include diagnosis, prevention and treatment of diseases and clinical conditions associated with KIAA0391. PRO1386 (Accession NM_{—}031269) is another VGAM181 host target gene. PRO1386 BINDING SITE is HOST TARGET binding site found in the 3′ untranslated region of mRNA encoded by PRO1386, corresponding to a HOST TARGET binding site such as BINDING SITE I, BINDING SITE II or BINDING SITE III. Table 2 illustrates the complementarity of the nucleotide sequences of PRO1386 BINDING SITE, designated SEQ ID:2188, to the nucleotide sequence of VGAM181 RNA, herein designated VGAM RNA, also designated SEQ ID:516.

Another function of VGAM181 is therefore inhibition of PRO1386 (Accession NM_{—}031269). Accordingly, utilities of VGAM181 include diagnosis, prevention and treatment of diseases and clinical conditions associated with PRO1386. RNA Binding Motif, Single Stranded Interacting Protein 1 (RBMS1, Accession NM_{—}016837) is another VGAM181 host target gene. RBMS1 BINDING SITE1 through RBMS1 BINDING SITE4 are HOST TARGET binding sites found in untranslated regions of mRNA encoded by RBMS1, corresponding to HOST TARGET binding sites such as BINDING SITE I, BINDING SITE II or BINDING SITE III. Table 2 illustrates the complementarity of the nucleotide sequences of RBMS1 BINDING SITE1 through RBMS1 BINDING SITE4, designated SEQ ID: 1711, SEQ ID: 1712, SEQ ID: 1713 and SEQ ID:970 respectively, to the nucleotide sequence of VGAM181 RNA, herein designated VGAM RNA, also designated SEQ ID:516.

Another function of VGAM181 is therefore inhibition of RNA Binding Motif, Single Stranded Interacting Protein 1 (RBMS1, Accession NM_{—}016837). Accordingly, utilities of VGAM181 include diagnosis, prevention and treatment of diseases and clinical conditions associated with RBMS1. SGP28 (Accession NM_{—}006061) is another VGAM181 host target gene. SGP28 BINDING SITE is HOST TARGET binding site found in the 3′ untranslated region of mRNA encoded by SGP28, corresponding to a HOST TARGET binding site such as BINDING SITE I, BINDING SITE II or BINDING SITE III. Table 2 illustrates the complementarity of the nucleotide sequences of SGP28 BINDING SITE, designated SEQ ID:1270, to the nucleotide sequence of VGAM181 RNA, herein designated VGAM RNA, also designated SEQ ID:516.

Another function of VGAM181 is therefore inhibition of SGP28 (Accession NM_{—}006061). Accordingly, utilities of VGAM181 include diagnosis, prevention and treatment of diseases and clinical conditions associated with SGP28. Suppression of Tumorigenicity 7 Like (ST7L, Accession NM_{—}017744) is another VGAM181 host target gene. ST7L BINDING SITE1 through ST7L BINDING SITE3 are HOST TARGET binding sites found in untranslated regions of mRNA encoded by ST7L, corresponding to HOST TARGET binding sites such as BINDING SITE I, BINDING SITE II or BINDING SITE III. Table 2 illustrates the complementarity of the nucleotide sequences of ST7L BINDING SITE1 through ST7L BINDING SITE3, designated SEQ ID:1746, SEQ ID:2456 and SEQ ID:2475 respectively, to the nucleotide sequence of VGAM181 RNA, herein designated VGAM RNA, also designated SEQ ID:516.

Another function of VGAM181 is therefore inhibition of Suppression of Tumorigenicity 7 Like (ST7L, Accession NM_{—}017744). Accordingly, utilities of VGAM181 include diagnosis, prevention and treatment of diseases and clinical conditions associated with ST7L. LOC157507 (Accession XM_{—}088312) is another VGAM181 host target gene. LOC157507 BINDING SITE is HOST TARGET binding site found in the 5′ untranslated region of mRNA encoded by LOC157507, corresponding to a HOST TARGET binding site such as BINDING SITE I, BINDING SITE II or BINDING SITE III. Table 2 illustrates the complementarity of the nucleotide sequences of LOC157507 BINDING SITE, designated SEQ ID:3202, to the nucleotide sequence of VGAM181 RNA, herein designated VGAM RNA, also designated SEQ ID:516.

Another function of VGAM181 is therefore inhibition of LOC157507 (Accession XM_{—}088312). Accordingly, utilities of VGAM181 include diagnosis, prevention and treatment of diseases and clinical conditions associated with LOC157507. LOC158158 (Accession XM_{—}088494) is another VGAM181 host target gene. LOC158158 BINDING SITE is HOST TARGET binding site found in the 3′ untranslated region of mRNA encoded by LOC158158, corresponding to a HOST TARGET binding site such as BINDING SITE I, BINDING SITE II or BINDING SITE III. Table 2 illustrates the complementarity of the nucleotide sequences of LOC158158 BINDING SITE, designated SEQ ID:3211, to the nucleotide sequence of VGAM181 RNA, herein designated VGAM RNA, also designated SEQ ID:516.

Another function of VGAM181 is therefore inhibition of LOC158158 (Accession XM_{—}088494). Accordingly, utilities of VGAM181 include diagnosis, prevention and treatment of diseases and clinical conditions associated with LOC158158. LOC219730 (Accession XM_{—}167580) is another VGAM181 host target gene. LOC219730 BINDING SITE is HOST TARGET binding site found in the 3′ untranslated region of mRNA encoded by LOC219730, corresponding to a HOST TARGET binding site such as BINDING SITE I, BINDING SITE II or BINDING SITE III. Table 2 illustrates the complementarity of the nucleotide sequences of LOC219730 BINDING SITE, designated SEQ ID:3588, to the nucleotide sequence of VGAM181 RNA, herein designated VGAM RNA, also designated SEQ ID:516.

Another function of VGAM181 is therefore inhibition of LOC219730 (Accession XM_{—}167580). Accordingly, utilities of VGAM181 include diagnosis, prevention and treatment of diseases and clinical conditions associated with LOC219730. LOC255158 (Accession XM_{—}171213) is another VGAM181 host target gene. LOC255158 BINDING SITE is HOST TARGET binding site found in the 3′ untranslated region of mRNA encoded by LOC255158, corresponding to a HOST TARGET binding site such as BINDING SITE I, BINDING SITE II or BINDING SITE III. Table 2 illustrates the complementarity of the nucleotide sequences of LOC255158 BINDING SITE, designated SEQ ID:3693, to the nucleotide sequence of VGAM181 RNA, herein designated VGAM RNA, also designated SEQ ID:516.

Another function of VGAM181 is therefore inhibition of LOC255158 (Accession XM_{—}171213). Accordingly, utilities of VGAM181 include diagnosis, prevention and treatment of diseases and clinical conditions associated with LOC255158. FIG. 1 further provides a conceptual description of a novel bioinformatically detected viral gene of the present invention, referred to here as Viral Genomic Address Messenger 182 (VGAM182) viral gene, which modulates expression of respective host target genes thereof, the function and utility of which host target genes is known in the art.

VGAM182 is a novel bioinformatically detected regulatory, non protein coding, viral micro RNA (miRNA) gene. The method by which VGAM182 was detected is described hereinabove with reference to FIGS. 1-8.

VGAM182 gene, herein designated VGAM GENE, is a viral gene contained in the genome of Vaccinia Virus. VGAM182 host target gene, herein designated VGAM HOST TARGET GENE, is a human gene contained in the human genome.

VGAM182 gene encodes a VGAM182 precursor RNA, herein designated VGAM PRECURSOR RNA. Similar to other miRNA genes, and unlike most ordinary genes, VGAM182 precursor RNA does not encode a protein. A nucleotide sequence identical or highly similar to the nucleotide sequence of VGAM182 precursor RNA is designated SEQ ID:168, and is provided hereinbelow with reference to the sequence listing part. Nucleotide sequence SEQ ID: 168 is located at position 96777 relative to the genome of Vaccinia Virus.

VGAM182 precursor RNA folds onto itself, forming VGAM182 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM182 folded precursor RNA into VGAM182 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM182 RNA is designated SEQ ID:517, and is provided hereinbelow with reference to the sequence listing part.

VGAM182 host target gene, herein designated VGAM HOST TARGET GENE, encodes a corresponding messenger RNA, VGAM182 host target RNA, herein designated VGAM HOST TARGET RNA. VGAM182 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM182 RNA, herein designated VGAM RNA, binds complementarily to one or more host target binding sites located in untranslated regions of VGAM182 host target RNA, herein designated VGAM HOST TARGET RNA. This complementary binding is due to the fact that the nucleotide sequence of VGAM182 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM182 RNA, herein designated VGAM RNA, may have a different number of host target binding sites in untranslated regions of a VGAM182 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM182 RNA, herein designated VGAM RNA, to host target binding sites on VGAM182 host target RNA, herein designated VGAM HOST TARGET RNA, such as BINDING SITE I, BINDING SITE II and BINDING SITE III, inhibits translation of VGAM182 host target RNA into VGAM182 host target protein, herein designated VGAM HOST TARGET PROTEIN. VGAM host target protein is therefore outlined by a broken line.

It is appreciated that VGAM182 host target gene, herein designated VGAM HOST TARGET GENE, in fact represents a plurality of VGAM182 host target genes. The mRNA of each one of this plurality of VGAM182 host target genes comprises one or more host target binding sites, each having a nucleotide sequence which is at least partly complementary to VGAM182 RNA, herein designated VGAM RNA, and which when bound by VGAM182 RNA causes inhibition of translation of respective one or more VGAM182 host target proteins.

It is further appreciated by one skilled in the art that the mode of translational inhibition illustrated by FIG. 1 with specific reference to translational inhibition exerted by VGAM182 gene, herein designated VGAM GENE, on one or more VGAM182 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM182 is inhibition of expression of host target genes, as part of a novel viral mechanism of attacking a host. Accordingly, utilities of VGAM182 include diagnosis, prevention and treatment of viral infection by Vaccinia Virus. Specific functions, and accordingly utilities, of VGAM182 correlate with, and may be deduced from, the identity of the host target genes which VGAM182 binds and inhibits, and the function of these host target genes, as elaborated hereinbelow.

Nucleotide sequences of the VGAM182 precursor RNA, herein designated VGAM PRECURSOR RNA, and of the ‘diced’ VGAM182 RNA, herein designated VGAM RNA, and a schematic representation of the secondary folding of VGAM182 folded precursor RNA, herein designated VGAM FOLDED PRECURSOR RNA, of VGAM182 are further described hereinbelow with reference to Table 1.

Nucleotide sequences of host target binding sites, such as BINDING SITE-I, BINDING SITE-II and BINDING SITE-III of FIG. 1, found on VGAM182 host target RNA, and schematic representation of the complementarity of each of these host target binding sites to VGAM182 RNA, herein designated VGAM RNA, are described hereinbelow with reference to Table 2.

As mentioned hereinabove with reference to FIG. 1, a function of VGAM182 gene, herein designated VGAM is inhibition of expression of VGAM182 target genes. It is appreciated that specific functions, and accordingly utilities, of VGAM182 correlate with, and may be deduced from, the identity of the target genes which VGAM182 binds and inhibits, and the function of these target genes, as elaborated hereinbelow.

Protein Tyrosine Phosphatase, Receptor Type, M (PTPRM, Accession NM_{—}002845) is a VGAM182 host target gene. PTPRM BINDING SITE is HOST TARGET binding site found in the 3′ untranslated region of mRNA encoded by PTPRM, corresponding to a HOST TARGET binding site such as BINDING SITE I, BINDING SITE II or BINDING SITE III. Table 2 illustrates the complementarity of the nucleotide sequences of PTPRM BINDING SITE, designated SEQ ID:963, to the nucleotide sequence of VGAM182 RNA, herein designated VGAM RNA, also designated SEQ ID:517.

A function of VGAM182 is therefore inhibition of Protein Tyrosine Phosphatase, Receptor Type, M (PTPRM, Accession NM_{—}002845). Accordingly, utilities of VGAM182 include diagnosis, prevention and treatment of diseases and clinical conditions associated with PTPRM. HCA4 (Accession NM_{—}138704) is another VGAM182 host target gene. HCA4 BINDING SITE1 and HCA4 BINDING SITE2 are HOST TARGET binding sites found in untranslated regions of mRNA encoded by HCA4, corresponding to HOST TARGET binding sites such as BINDING SITE I, BINDING SITE II or BINDING SITE III. Table 2 illustrates the complementarity of the nucleotide sequences of HCA4 BINDING SITE1 and HCA4 BINDING SITE2, designated SEQ ID:2450 and SEQ ID:3086 respectively, to the nucleotide sequence of VGAM182 RNA, herein designated VGAM RNA, also designated SEQ ID:517.

Another function of VGAM182 is therefore inhibition of HCA4 (Accession NM_{—}138704). Accordingly, utilities of VGAM182 include diagnosis, prevention and treatment of diseases and clinical conditions associated with HCA4. HDAC9-PENDING (Accession NM_{—}014707) is another VGAM183 host target gene. HDAC9-PENDING BINDING SITE is HOST TARGET binding site found in the 3′ untranslated region of mRNA encoded by HDAC9-PENDING, corresponding to a HOST TARGET binding site such as BINDING SITE I, BINDING SITE II or BINDING SITE III. Table 2 illustrates the complementarity of the nucleotide sequences of HDAC9-PENDING BINDING SITE, designated SEQ ID: 1527, to the nucleotide sequence of VGAM183 RNA, herein designated VGAM RNA, also designated SEQ ID:518.

Another function of VGAM183 is therefore inhibition of HDAC9-PENDING (Accession NM_{—}014707). Accordingly, utilities of VGAM183 include diagnosis, prevention and treatment of diseases and clinical conditions associated with HDAC9-PENDING. KIAA0232 (Accession XM_{—}052627) is another VGAM183 host target gene. KIAA0232 BINDING SITE is HOST TARGET binding site found in the 3′ untranslated region of mRNA encoded by KIAA0232, corresponding to a HOST TARGET binding site such as BINDING SITE I, BINDING SITE II or BINDING SITE III. Table 2 illustrates the complementarity of the nucleotide sequences of KIAA0232 BINDING SITE, designated SEQ ID:2958, to the nucleotide sequence of VGAM183 RNA, herein designated VGAM RNA, also designated SEQ ID:518.

Another function of VGAM183 is therefore inhibition of KIAA0232 (Accession XM_{—}052627). Accordingly, utilities of VGAM183 include diagnosis, prevention and treatment of diseases and clinical conditions associated with KIAA0232. KIAA1010 (Accession XM_{—}050742) is another VGAM183 host target gene. KIAA1010 BINDING SITE is HOST TARGET binding site found in the 3′ untranslated region of mRNA encoded by KIAA1010, corresponding to a HOST TARGET binding site such as BINDING SITE I, BINDING SITE II or BINDING SITE III. Table 2 illustrates the complementarity of the nucleotide sequences of KIAA1010 BINDING SITE, designated SEQ ID:2933, to the nucleotide sequence of VGAM183 RNA, herein designated VGAM RNA, also designated SEQ ID:518.

Another function of VGAM183 is therefore inhibition of KIAA1010 (Accession XM_{—}050742). Accordingly, utilities of VGAM183 include diagnosis, prevention and treatment of diseases and clinical conditions associated with KIAA1010. LOC219894 (Accession XM_{—}167782) is another VGAM183 host target gene. LOC219894 BINDING SITE is HOST TARGET binding site found in the 3′ untranslated region of mRNA encoded by LOC219894, corresponding to a HOST TARGET binding site such as BINDING SITE I, BINDING SITE II or BINDING SITE III. Table 2 illustrates the complementarity of the nucleotide sequences of LOC219894 BINDING SITE, designated SEQ ID:3596, to the nucleotide sequence of VGAM183 RNA, herein designated VGAM RNA, also designated SEQ ID:518.

Another function of VGAM183 is therefore inhibition of LOC219894 (Accession XM_{—}167782). Accordingly, utilities of VGAM183 include diagnosis, prevention and treatment of diseases and clinical conditions associated with LOC219894. FIG. 1 further provides a conceptual description of a novel bioinformatically detected viral gene of the present invention, referred to here as Viral Genomic Address Messenger 184 (VGAM184) viral gene, which modulates expression of respective host target genes thereof, the function and utility of which host target genes is known in the art.

VGAM184 is a novel bioinformatically detected regulatory, non protein coding, viral micro RNA (miRNA) gene. The method by which VGAM184 was detected is described hereinabove with reference to FIGS. 1-8.

VGAM184 gene, herein designated VGAM GENE, is a viral gene contained in the genome of Vaccinia Virus. VGAM184 host target gene, herein designated VGAM HOST TARGET GENE, is a human gene contained in the human genome.

VGAM184 gene encodes a VGAM184 precursor RNA, herein designated VGAM PRECURSOR RNA. Similar to other miRNA genes, and unlike most ordinary genes, VGAM184 precursor RNA does not encode a protein. A nucleotide sequence identical or highly similar to the nucleotide sequence of VGAM184 precursor RNA is designated SEQ ID:170, and is provided hereinbelow with reference to the sequence listing part. Nucleotide sequence SEQ ID:170 is located at position 95224 relative to the genome of Vaccinia Virus.

VGAM184 precursor RNA folds onto itself, forming VGAM184 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM184 folded precursor RNA into VGAM184 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM184 RNA is designated SEQ ID:519, and is provided hereinbelow with reference to the sequence listing part.

VGAM184 host target gene, herein designated VGAM HOST TARGET GENE, encodes a corresponding messenger RNA, VGAM184 host target RNA, herein designated VGAM HOST TARGET RNA. VGAM184 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM184 RNA, herein designated VGAM RNA, binds complementarily to one or more host target binding sites located in untranslated regions of VGAM184 host target RNA, herein designated VGAM HOST TARGET RNA. This complementary binding is due to the fact that the nucleotide sequence of VGAM184 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM184 RNA, herein designated VGAM RNA, may have a different number of host target binding sites in untranslated regions of a VGAM184 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM184 RNA, herein designated VGAM RNA, to host target binding sites on VGAM184 host target RNA, herein designated VGAM HOST TARGET RNA, such as BINDING SITE I, BINDING SITE II and BINDING SITE III, inhibits translation of VGAM184 host target RNA into VGAM184 host target protein, herein designated VGAM HOST TARGET PROTEIN. VGAM host target protein is therefore outlined by a broken line.

It is appreciated that VGAM184 host target gene, herein designated VGAM HOST TARGET GENE, in fact represents a plurality of VGAM184 host target genes. The mRNA of each one of this plurality of VGAM184 host target genes comprises one or more host target binding sites, each having a nucleotide sequence which is at least partly complementary to VGAM184 RNA, herein designated VGAM RNA, and which when bound by VGAM184 RNA causes inhibition of translation of respective one or more VGAM184 host target proteins.

It is further appreciated by one skilled in the art that the mode of translational inhibition illustrated by FIG. 1 with specific reference to translational inhibition exerted by VGAM184 gene, herein designated VGAM GENE, on one or more VGAM184 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM184 is inhibition of expression of host target genes, as part of a novel viral mechanism of attacking a host. Accordingly, utilities of VGAM184 include diagnosis, prevention and treatment of viral infection by Vaccinia Virus. Specific functions, and accordingly utilities, of VGAM184 correlate with, and may be deduced from, the identity of the host target genes which VGAM184 binds and inhibits, and the function of these host target genes, as elaborated hereinbelow.

Nucleotide sequences of the VGAM184 precursor RNA, herein designated VGAM PRECURSOR RNA, and of the ‘diced’ VGAM184 RNA, herein designated VGAM RNA, and a schematic representation of the secondary folding of VGAM184 folded precursor RNA, herein designated VGAM FOLDED PRECURSOR RNA, of VGAM184 are further described hereinbelow with reference to Table 1.

Nucleotide sequences of host target binding sites, such as BINDING SITE-I, BINDING SITE-II and BINDING SITE-III of FIG. 1, found on VGAM184 host target RNA, and schematic representation of the complementarity of each of these host target binding sites to VGAM184 RNA, herein designated VGAM RNA, are described hereinbelow with reference to Table 2.

As mentioned hereinabove with reference to FIG. 1, a function of VGAM184 gene, herein designated VGAM is inhibition of expression of VGAM184 target genes. It is appreciated that specific functions, and accordingly utilities, of VGAM184 correlate with, and may be deduced from, the identity of the target genes which VGAM184 binds and inhibits, and the function of these target genes, as elaborated hereinbelow.

poly(A) Binding Protein, Cytoplasmic 4 (inducible form) (PABPC4, Accession NM_{—}003819) is a VGAM184 host target gene. PABPC4 BINDING SITE is HOST TARGET binding site found in the 3′ untranslated region of mRNA encoded by PABPC4, corresponding to a HOST TARGET binding site such as BINDING SITE I, BINDING SITE II or BINDING SITE III. Table 2 illustrates the complementarity of the nucleotide sequences of PABPC4 BINDING SITE, designated SEQ ID: 1064, to the nucleotide sequence of VGAM184 RNA, herein designated VGAM RNA, also designated SEQ ID:519.

A function of VGAM184 is therefore inhibition of poly(A) Binding Protein, Cytoplasmic 4 (inducible form) (PABPC4, Accession NM_{—}003819), a gene which involves in cytoplasmic regulatory processes of mRNA metabolism. Accordingly, utilities of VGAM184 include diagnosis, prevention and treatment of diseases and clinical conditions associated with PABPC4. The function of PABPC4 has been established by previous studies. PABPs bind to the poly(A) tail present at the 3-prime ends of most eukaryotic mRNAs. Yang et al. (1995) isolated an activation-induced T-cell mRNA encoding a protein similar to PABPC1. The predicted 644-amino acid protein, which they designated iPABP (inducible PABP), shares 79% sequence identity with PABPC1. Both proteins contain 4 RNA-binding domains and proline-rich C termini, and both have very similar RNA-binding properties. Using immunofluorescence, Yang et al. (1995) determined that iPABP localizes primarily to the cytoplasm. Northern blot analysis revealed that iPABP was expressed as a 3.2-kb mRNA in all tissues tested. Activation of T cells increased iPABP mRNA levels in T cells approximately 5-fold. The authors suggested that iPABP might be necessary for regulation of stability of labile mRNA species in activated T cells. Platelets are activated in response to vascular injury. Activated platelets express molecules that execute many of the complex cellular biochemical processes that staunch the loss of blood and begin the process of vascular repair. Houng et al. (1997) identified iPABP as an antigen, APP1 (activated-platelet protein-1), expressed on thrombin-activated rabbit platelets. They isolated rabbit and human APP1 cDNAs. The predicted human and rabbit proteins are 99% identical. Houng et al. (1997) stated that, given its similarity to PABPC1, APP1 may be involved in the regulation of protein translation in platelets and megakaryocytes or may participate in the binding or stabilization of polyadenylates in platelet dense granules.

Full details of the abovementioned studies are described in the following publications, the disclosure of which are hereby incorporated by reference:
Yang, H.; Duckett, C. S.; Lindsten, T.: iPABP, an inducible poly(A)-binding protein detected in activated human T cells. Molec. Cell. Biol. 15: 6770-6776, 1995.; and
Houng, A. K.; Maggini, L.; Clement, C. Y.; Reed, G. L.: Identification and structure of activated-platelet protein-1, a protein with RNA-binding domain motifs that is expressed by activ.

Further studies establishing the function and utilities of PABPC4 are found in John Hopkins OMIM database record ID 603407, and in sited publications numbered 1205-1207 listed in the bibliography section hereinbelow, which are also hereby incorporated by reference. SH3-domain GRB2-like 2 (SH3GL2, Accession NM_{—}003026) is another VGAM184 host target gene. SH3GL2 BINDING SITE is HOST TARGET binding site found in the 3′ untranslated region of mRNA encoded by SH3GL2, corresponding to a HOST TARGET binding site such as BINDING SITE I, BINDING SITE II or BINDING SITE III. Table 2 illustrates the complementarity of the nucleotide sequences of SH3GL2 BINDING SITE, designated SEQ ID:983, to the nucleotide sequence of VGAM184 RNA, herein designated VGAM RNA, also designated SEQ ID:519.

Another function of VGAM184 is therefore inhibition of SH3-domain GRB2-like 2 (SH3GL2, Accession NM_{—}003026), a gene which plays a role in synaptic vesicle recycling, in particular in clathrin-mediated vesicle endocytosis. Accordingly, utilities of VGAM184 include diagnosis, prevention and treatment of diseases and clinical conditions associated with SH3GL2. The function of SH3GL2 and its association with various diseases and clinical conditions, has been established by previous studies, as described hereinabove with reference to VGAM84.1 MP-2 (Accession NM_{—}006548) is another VGAM184 host target gene. IMP-2 BINDING SITE is HOST TARGET binding site found in the 3′ untranslated region of mRNA encoded by IMP-2, corresponding to a HOST TARGET binding site such as BINDING SITE I, BINDING SITE II or BINDING SITE III. Table 2 illustrates the complementarity of the nucleotide sequences of IMP-2 BINDING SITE, designated SEQ ID:1309, to the nucleotide sequence of VGAM184 RNA, herein designated VGAM RNA, also designated SEQ ID:519.

Another function of VGAM184 is therefore inhibition of IMP-2 (Accession NM_{—}006548). Accordingly, utilities of VGAM184 include diagnosis, prevention and treatment of diseases and clinical conditions associated with IMP-2. KIAA0872 (Accession NM_{—}014940) is another VGAM184 host target gene. KIAA0872 BINDING SITE is HOST TARGET binding site found in the 3′ untranslated region of mRNA encoded by KIAA0872, corresponding to a HOST TARGET binding site such as BINDING SITE I, BINDING SITE II or BINDING SITE III. Table 2 illustrates the complementarity of the nucleotide sequences of KIAA0872 BINDING SITE, designated SEQ ID:1593, to the nucleotide sequence of VGAM184 RNA, herein designated VGAM RNA, also designated SEQ ID:519.

Another function of VGAM184 is therefore inhibition of KIAA0872 (Accession NM_{—}014940). Accordingly, utilities of VGAM184 include diagnosis, prevention and treatment of diseases and clinical conditions associated with KIAA0872. MGC19570 (Accession NM_{—}145063) is another VGAM184 host target gene. MGC19570 BINDING SITE is HOST TARGET binding site found in the 5′ untranslated region of mRNA encoded by MGC19570, corresponding to a HOST TARGET binding site such as BINDING SITE I, BINDING SITE II or BINDING SITE III. Table 2 illustrates the complementarity of the nucleotide sequences of MGC19570 BINDING SITE, designated SEQ ID:2514, to the nucleotide sequence of VGAM184 RNA, herein designated VGAM RNA, also designated SEQ ID:519.

Another function of VGAM184 is therefore inhibition of MGC19570 (Accession NM_{—}145063). Accordingly, utilities of VGAM184 include diagnosis, prevention and treatment of diseases and clinical conditions associated with MGC19570. Protein Tyrosine Phosphatase, Non-receptor Type 4 (megakaryocyte) (PTPN4, Accession NM_{—}002830) is another VGAM184 host target gene. PTPN4 BINDING SITE is HOST TARGET binding site found in the 3′ untranslated region of mRNA encoded by PTPN4, corresponding to a HOST TARGET binding site such as BINDING SITE I, BINDING SITE II or BINDING SITE III. Table 2 illustrates the complementarity of the nucleotide sequences of PTPN4 BINDING SITE, designated SEQ ID:961, to the nucleotide sequence of VGAM184 RNA, herein designated VGAM RNA, also designated SEQ ID:519.

Another function of VGAM184 is therefore inhibition of Protein Tyrosine Phosphatase, Non-receptor Type 4 (megakaryocyte) (PTPN4, Accession NM_{—}002830). Accordingly, utilities of VGAM184 include diagnosis, prevention and treatment of diseases and clinical conditions associated with PTPN4. Triple Homeobox 1 (TIX1, Accession XM_{—}029734) is another VGAM184 host target gene. TIX1 BINDING SITE is HOST TARGET binding site found in the 5′ untranslated region of mRNA encoded by TIX1, corresponding to a HOST TARGET binding site such as BINDING SITE I, BINDING SITE II or BINDING SITE III. Table 2 illustrates the complementarity of the nucleotide sequences of TIX1 BINDING SITE, designated SEQ ID:2604, to the nucleotide sequence of VGAM184 RNA, herein designated VGAM RNA, also designated SEQ ID:519.

Another function of VGAM184 is therefore inhibition of Triple Homeobox 1 (TIX1, Accession XM_{—}029734). Accordingly, utilities of VGAM184 include diagnosis, prevention and treatment of diseases and clinical conditions associated with TIX1. FIG. 1 further provides a conceptual description of a novel bioinformatically detected viral gene of the present invention, referred to here as Viral Genomic Address Messenger 185 (VGAM185) viral gene, which modulates expression of respective host target genes thereof, the function and utility of which host target genes is known in the art.

VGAM185 is a novel bioinformatically detected regulatory, non protein coding, viral micro RNA (miRNA) gene. The method by which VGAM185 was detected is described hereinabove with reference to FIGS. 1-8.

VGAM185 gene, herein designated VGAM GENE, is a viral gene contained in the genome of Vaccinia Virus. VGAM185 host target gene, herein designated VGAM HOST TARGET GENE, is a human gene contained in the human genome.

VGAM185 gene encodes a VGAM185 precursor RNA, herein designated VGAM PRECURSOR RNA. Similar to other miRNA genes, and unlike most ordinary genes, VGAM185 precursor RNA does not encode a protein. A nucleotide sequence identical or highly similar to the nucleotide sequence of VGAM185 precursor RNA is designated SEQ ID:171, and is provided hereinbelow with reference to the sequence listing part. Nucleotide sequence SEQ ID:171 is located at position 97665 relative to the genome of Vaccinia Virus.

VGAM185 precursor RNA folds onto itself, forming VGAM185 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM185 folded precursor RNA into VGAM185 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM185 RNA is designated SEQ ID:520, and is provided hereinbelow with reference to the sequence listing part.

VGAM185 host target gene, herein designated VGAM HOST TARGET GENE, encodes a corresponding messenger RNA, VGAM185 host target RNA, herein designated VGAM HOST TARGET RNA. VGAM185 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM185 RNA, herein designated VGAM RNA, binds complementarily to one or more host target binding sites located in untranslated regions of VGAM185 host target RNA, herein designated VGAM HOST TARGET RNA. This complementary binding is due to the fact that the nucleotide sequence of VGAM185 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM185 RNA, herein designated VGAM RNA, may have a different number of host target binding sites in untranslated regions of a VGAM185 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM185 RNA, herein designated VGAM RNA, to host target binding sites on VGAM185 host target RNA, herein designated VGAM HOST TARGET RNA, such as BINDING SITE I, BINDING SITE II and BINDING SITE III, inhibits translation of VGAM185 host target RNA into VGAM185 host target protein, herein designated VGAM HOST TARGET PROTEIN. VGAM host target protein is therefore outlined by a broken line.

It is appreciated that VGAM185 host target gene, herein designated VGAM HOST TARGET GENE, in fact represents a plurality of VGAM185 host target genes. The mRNA of each one of this plurality of VGAM185 host target genes comprises one or more host target binding sites, each having a nucleotide sequence which is at least partly complementary to VGAM185 RNA, herein designated VGAM RNA, and which when bound by VGAM185 RNA causes inhibition of translation of respective one or more VGAM185 host target proteins.

It is further appreciated by one skilled in the art that the mode of translational inhibition illustrated by FIG. 1 with specific reference to translational inhibition exerted by VGAM185 gene, herein designated VGAM GENE, on one or more VGAM185 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM185 is inhibition of expression of host target genes, as part of a novel viral mechanism of attacking a host. Accordingly, utilities of VGAM185 include diagnosis, prevention and treatment of viral infection by Vaccinia Virus. Specific functions, and accordingly utilities, of VGAM185 correlate with, and may be deduced from, the identity of the host target genes which VGAM185 binds and inhibits, and the function of these host target genes, as elaborated hereinbelow.

Nucleotide sequences of the VGAM185 precursor RNA, herein designated VGAM PRECURSOR RNA, and of the ‘diced’ VGAM185 RNA, herein designated VGAM RNA, and a schematic representation of the secondary folding of VGAM185 folded precursor RNA, herein designated VGAM FOLDED PRECURSOR RNA, of VGAM185 are further described hereinbelow with reference to Table 1.

Nucleotide sequences of host target binding sites, such as BINDING SITE-I, BINDING SITE-II and BINDING SITE-III of FIG. 1, found on VGAM185 host target RNA, and schematic representation of the complementarity of each of these host target binding sites to VGAM185 RNA, herein designated VGAM RNA, are described hereinbelow with reference to Table 2.

As mentioned hereinabove with reference to FIG. 1, a function of VGAM185 gene, herein designated VGAM is inhibition of expression of VGAM185 target genes. It is appreciated that specific functions, and accordingly utilities, of VGAM185 correlate with, and may be deduced from, the identity of the target genes which VGAM185 binds and inhibits, and the function of these target genes, as elaborated hereinbelow.

Pregnancy Specific Beta-1-glycoprotein 5 (PSG5, Accession NM_{—}002781) is a VGAM185 host target gene. PSG5 BINDING SITE is HOST TARGET binding site found in the 3′ untranslated region of mRNA encoded by PSG5, corresponding to a HOST TARGET binding site such as BINDING SITE I, BINDING SITE II or BINDING SITE III. Table 2 illustrates the complementarity of the nucleotide sequences of PSG5 BINDING SITE, designated SEQ ID:955, to the nucleotide sequence of VGAM185 RNA, herein designated VGAM RNA, also designated SEQ ID:520.

A function of VGAM185 is therefore inhibition of Pregnancy Specific Beta-1-glycoprotein 5 (PSG5, Accession NM_{—}002781), a gene which is a member of the pregnancy-specific glycoprotein (PSG) and CEA families. Accordingly, utilities of VGAM185 include diagnosis, prevention and treatment of diseases and clinical conditions associated with PSG5. The function of PSG5 has been established by previous studies. The human pregnancy-specific glycoproteins (PSGs) are a group of molecules that are mainly produced by the placental syncytiotrophoblasts during pregnancy. PSGs comprise a subgroup of the carcinoembryonic antigen (CEA) family, which belongs to the immunoglobulin super family. See PSG3 (OMIM Ref. No. 176392) for additional information about PSGs. Teglund et al. (1994) found that the PSG5 gene contains 6 exons. Studies by several groups resulted in the mapping of the CEA gene family to 19q13.1-q13.2 (Thompson et al., 1990; Thompson et al., 1992; Tynan et al., 1992; Trask et al., 1993). The PSG subgroup is located telomeric of the CEA subgroup, and together they span approximately 1.1 to 1.2 Mb (Brandriff et al., 1992; Tynan et al., 1992). Using a high-resolution restriction fragment fingerprinting technique, Olsen et al. (1994) assembled 256 cosmids spanning the PSG region on 19q13.2 into a single 700-kb contig. FISH to sperm pronuclei and cosmid walking experiments indicated that this PSG contig is telomeric of CGM8 at the telomeric end of the CEA subgroup gene cluster. Detailed restriction mapping and hybridization with gene-specific probes indicated that the order of the 11 PSG genes in the contig is cen—PSG3—PSG8 (OMIM Ref. No. 176397)—PSG12 (PSG10; 176399)—PSG1 (OMIM Ref. No. 176390)—PSG6 (OMIM Ref. No. 176395)—PSG7 (OMIM Ref. No. 176396)—PSG13 (PSG11; 176401)—PSG2 (OMIM Ref. No. 176391)—PSG5—PSG4 (OMIM Ref. No. 176393)—PSG11 (PSG9; 176398)—tel. The PSG genes are tandemly oriented in a 5-prime to 3-prime direction from telomere to centromere. The CEA subgroup gene CGM11 is located at the telomeric end of the PSG gene cluster, and 6 genes belonging to a third CEA family subgroup, namely CGM13 through CGM18 (later OMIM Ref. No. 109770), are interspersed among the PSG genes. Nomenclature: Beauchemin et al. (1999) provided a revised nomenclature for the CEA gene family. Based on this nomenclature, the CEA family is composed of the PSG sub family, the CEACAM sub family (see OMIM Ref. No. 109770), and the CEACAM pseudogene (CEACAMP) sub family (see OMIM Ref. No. 109770). PSG11, PSG12, and PSG13 were renamed PSG9, PSG10, and PSG11, respectively.

Full details of the abovementioned studies are described in the following publications, the disclosure of which are hereby incorporated by reference:
Beauchemin, N.; Draber, P.; Dveksler, G.; Gold, P.; Gray-Owen, S.; Grunert, F.; Hammarstrom, S.; Holmes, K. V.; Karlsson, A.; Kuroki, M.; Lin, S.-H.; Lucka, L.; and 13 others: Redefined nomenclature for members of the carcinoembryonic antigen family. Exp. Cell Res. 252: 243-249, 1999.; and
Brandriff, B. F.; Gordon, L. A.; Tynan, K. T.; Olsen, A. S.; Mohrenweiser, H. W.; Fertitta, A.; Carrano, A. V.; Trask, B. J.: Order and genomic distances among members of the carcinoem.

Further studies establishing the function and utilities of PSG5 are found in John Hopkins OMIM database record ID 176394, and in sited publications numbered 503, 2456, 2467-2465, 246 and 2469 listed in the bibliography section hereinbelow, which are also hereby incorporated by reference. FAPP2 (Accession NM_{—}032639) is another VGAM185 host target gene. FAPP2 BINDING SITE is HOST TARGET binding site found in the 3′ untranslated region of mRNA encoded by FAPP2, corresponding to a HOST TARGET binding site such as BINDING SITE I, BINDING SITE II or BINDING SITE III. Table 2 illustrates the complementarity of the nucleotide sequences of FAPP2 BINDING SITE, designated SEQ ID:2267, to the nucleotide sequence of VGAM185 RNA, herein designated VGAM RNA, also designated SEQ ID:520.

Another function of VGAM185 is therefore inhibition of FAPP2 (Accession NM_{—}032639). Accordingly, utilities of VGAM185 include diagnosis, prevention and treatment of diseases and clinical conditions associated with FAPP2. LOC145820 (Accession XM_{—}085246) is another VGAM185 host target gene. LOC145820 BINDING SITE is HOST TARGET binding site found in the 3′ untranslated region of mRNA encoded by LOC145820, corresponding to a HOST TARGET binding site such as BINDING SITE I, BINDING SITE II or BINDING SITE III. Table 2 illustrates the complementarity of the nucleotide sequences of LOC145820 BINDING SITE, designated SEQ ID:3084, to the nucleotide sequence of VGAM185 RNA, herein designated VGAM RNA, also designated SEQ ID:520.

Another function of VGAM185 is therefore inhibition of LOC145820 (Accession XM_{—}085246). Accordingly, utilities of VGAM185 include diagnosis, prevention and treatment of diseases and clinical conditions associated with LOC145820. LOC83690 (Accession NM_{—}031461) is another VGAM185 host target gene. LOC83690 BINDING SITE is HOST TARGET binding site found in the 3′ untranslated region of mRNA encoded by LOC83690, corresponding to a HOST TARGET binding site such as BINDING SITE I, BINDING SITE II or BINDING SITE III. Table 2 illustrates the complementarity of the nucleotide sequences of LOC83690 BINDING SITE, designated SEQ ID:2202, to the nucleotide sequence of VGAM185 RNA, herein designated VGAM RNA, also designated SEQ ID:520.

Another function of VGAM185 is therefore inhibition of LOC83690 (Accession NM_{—}031461). Accordingly, utilities of VGAM185 include diagnosis, prevention and treatment of diseases and clinical conditions associated with LOC83690. FIG. 1 further provides a conceptual description of a novel bioinformatically detected viral gene of the present invention, referred to here as Viral Genomic Address Messenger 186 (VGAM186) viral gene, which modulates expression of respective host target genes thereof, the function and utility of which host target genes is known in the art.

VGAM186 is a novel bioinformatically detected regulatory, non protein coding, viral micro RNA (miRNA) gene. The method by which VGAM186 was detected is described hereinabove with reference to FIGS. 1-8.

VGAM186 gene, herein designated VGAM GENE, is a viral gene contained in the genome of Vaccinia Virus. VGAM186 host target gene, herein designated VGAM HOST TARGET GENE, is a human gene contained in the human genome.

VGAM186 gene encodes a VGAM186 precursor RNA, herein designated VGAM PRECURSOR RNA. Similar to other miRNA genes, and unlike most ordinary genes, VGAM186 precursor RNA does not encode a protein. A nucleotide sequence identical or highly similar to the nucleotide sequence of VGAM186 precursor RNA is designated SEQ ID:172, and is provided hereinbelow with reference to the sequence listing part. Nucleotide sequence SEQ ID:172 is located at position 99100 relative to the genome of Vaccinia Virus.

VGAM186 precursor RNA folds onto itself, forming VGAM186 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM186 folded precursor RNA into VGAM186 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 94%) nucleotide sequence of VGAM186 RNA is designated SEQ ID:521, and is provided hereinbelow with reference to the sequence listing part.

VGAM186 host target gene, herein designated VGAM HOST TARGET GENE, encodes a corresponding messenger RNA, VGAM186 host target RNA, herein designated VGAM HOST TARGET RNA. VGAM186 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM186 RNA, herein designated VGAM RNA, binds complementarily to one or more host target binding sites located in untranslated regions of VGAM186 host target RNA, herein designated VGAM HOST TARGET RNA. This complementary binding is due to the fact that the nucleotide sequence of VGAM186 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM186 RNA, herein designated VGAM RNA, may have a different number of host target binding sites in untranslated regions of a VGAM186 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM186 RNA, herein designated VGAM RNA, to host target binding sites on VGAM186 host target RNA, herein designated VGAM HOST TARGET RNA, such as BINDING SITE I, BINDING SITE II and BINDING SITE III, inhibits translation of VGAM186 host target RNA into VGAM186 host target protein, herein designated VGAM HOST TARGET PROTEIN. VGAM host target protein is therefore outlined by a broken line.

It is appreciated that VGAM186 host target gene, herein designated VGAM HOST TARGET GENE, in fact represents a plurality of VGAM186 host target genes. The mRNA of each one of this plurality of VGAM186 host target genes comprises one or more host target binding sites, each having a nucleotide sequence which is at least partly complementary to VGAM186 RNA, herein designated VGAM RNA, and which when bound by VGAM186 RNA causes inhibition of translation of respective one or more VGAM186 host target proteins.

It is further appreciated by one skilled in the art that the mode of translational inhibition illustrated by FIG. 1 with specific reference to translational inhibition exerted by VGAM186 gene, herein designated VGAM GENE, on one or more VGAM186 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM186 is inhibition of expression of host target genes, as part of a novel viral mechanism of attacking a host. Accordingly, utilities of VGAM186 include diagnosis, prevention and treatment of viral infection by Vaccinia Virus. Specific functions, and accordingly utilities, of VGAM186 correlate with, and may be deduced from, the identity of the host target genes which VGAM186 binds and inhibits, and the function of these host target genes, as elaborated hereinbelow.

Nucleotide sequences of the VGAM186 precursor RNA, herein designated VGAM PRECURSOR RNA, and of the ‘diced’ VGAM186 RNA, herein designated VGAM RNA, and a schematic representation of the secondary folding of VGAM186 folded precursor RNA, herein designated VGAM FOLDED PRECURSOR RNA, of VGAM186 are further described hereinbelow with reference to Table 1.

Nucleotide sequences of host target binding sites, such as BINDING SITE-I, BINDING SITE-II and BINDING SITE-III of FIG. 1, found on VGAM186 host target RNA, and schematic representation of the complementarity of each of these host target binding sites to VGAM186 RNA, herein designated VGAM RNA, are described hereinbelow with reference to Table 2.

As mentioned hereinabove with reference to FIG. 1, a function of VGAM186 gene, herein designated VGAM is inhibition of expression of VGAM186 target genes. It is appreciated that specific functions, and accordingly utilities, of VGAM186 correlate with, and may be deduced from, the identity of the target genes which VGAM186 binds and inhibits, and the function of these target genes, as elaborated hereinbelow.

Insulin-like Growth Factor 1 (somatomedin C) (IGF1, Accession NM_{—}000618) is a VGAM186 host target gene. IGF1 BINDING SITE is HOST TARGET binding site found in the 3′ untranslated region of mRNA encoded by IGF1, corresponding to a HOST TARGET binding site such as BINDING SITE I, BINDING SITE II or BINDING SITE III. Table 2 illustrates the complementarity of the nucleotide sequences of IGF1 BINDING SITE, designated SEQ ID:762, to the nucleotide sequence of VGAM186 RNA, herein designated VGAM RNA, also designated SEQ ID:521.

A function of VGAM186 is therefore inhibition of Insulin-like Growth Factor 1 (somatomedin C) (IGF1, Accession NM_{—}000618), a gene which are structurally and functionally related to insulin but have a much higher growth-promoting activity. Accordingly, utilities of VGAM186 include diagnosis, prevention and treatment of diseases and clinical conditions associated with IGF1. The function of IGF1 and its association with various diseases and clinical conditions, has been established by previous studies, as described hereinabove with reference to VGAM34. FLJ20035 (Accession NM_{—}017631) is another VGAM186 host target gene. FLJ20035 BINDING SITE is HOST TARGET binding site found in the 3′ untranslated region of mRNA encoded by FLJ20035, corresponding to a HOST TARGET binding site such as BINDING SITE I, BINDING SITE II or BINDING SITE III. Table 2 illustrates the complementarity of the nucleotide sequences of FLJ20035 BINDING SITE, designated SEQ ID:1733, to the nucleotide sequence of VGAM186 RNA, herein designated VGAM RNA, also designated SEQ ID:521.

Another function of VGAM186 is therefore inhibition of FLJ20035 (Accession NM_{—}017631). Accordingly, utilities of VGAM186 include diagnosis, prevention and treatment of diseases and clinical conditions associated with FLJ20035. FXYD Domain Containing Ion Transport Regulator 3 (FXYD3, Accession NM_{—}021910) is another VGAM186 host target gene. FXYD3 BINDING SITE1 and FXYD3 BINDING SITE2 are HOST TARGET binding sites found in untranslated regions of mRNA encoded by FXYD3, corresponding to HOST TARGET binding sites such as BINDING SITE I, BINDING SITE II or BINDING SITE III. Table 2 illustrates the complementarity of the nucleotide sequences of FXYD3 BINDING SITE1 and FXYD3 BINDING SITE2, designated SEQ ID: 1966 and SEQ ID: 1263 respectively, to the nucleotide sequence of VGAM186 RNA, herein designated VGAM RNA, also designated SEQ ID:521.

Another function of VGAM186 is therefore inhibition of FXYD Domain Containing Ion Transport Regulator 3 (FXYD3, Accession NM_{—}021910). Accordingly, utilities of VGAM186 include diagnosis, prevention and treatment of diseases and clinical conditions associated with FXYD3. FIG. 1 further provides a conceptual description of a novel bioinformatically detected viral gene of the present invention, referred to here as Viral Genomic Address Messenger 187 (VGAM187) viral gene, which modulates expression of respective host target genes thereof, the function and utility of which host target genes is known in the art.

VGAM187 is a novel bioinformatically detected regulatory, non protein coding, viral micro RNA (miRNA) gene. The method by which VGAM187 was detected is described hereinabove with reference to FIGS. 1-8.

VGAM187 gene, herein designated VGAM GENE, is a viral gene contained in the genome of Vaccinia Virus. VGAM187 host target gene, herein designated VGAM HOST TARGET GENE, is a human gene contained in the human genome.

VGAM187 gene encodes a VGAM187 precursor RNA, herein designated VGAM PRECURSOR RNA. Similar to other miRNA genes, and unlike most ordinary genes, VGAM187 precursor RNA does not encode a protein. A nucleotide sequence identical or highly similar to the nucleotide sequence of VGAM187 precursor RNA is designated SEQ ID:173, and is provided hereinbelow with reference to the sequence listing part. Nucleotide sequence SEQ ID:173 is located at position 98088 relative to the genome of Vaccinia Virus.

VGAM187 precursor RNA folds onto itself, forming VGAM187 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM187 folded precursor RNA into VGAM187 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM187 RNA is designated SEQ ID:522, and is provided hereinbelow with reference to the sequence listing part.

VGAM187 host target gene, herein designated VGAM HOST TARGET GENE, encodes a corresponding messenger RNA, VGAM187 host target RNA, herein designated VGAM HOST TARGET RNA. VGAM187 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM187 RNA, herein designated VGAM RNA, binds complementarily to one or more host target binding sites located in untranslated regions of VGAM187 host target RNA, herein designated VGAM HOST TARGET RNA. This complementary binding is due to the fact that the nucleotide sequence of VGAM187 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM187 RNA, herein designated VGAM RNA, may have a different number of host target binding sites in untranslated regions of a VGAM187 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM187 RNA, herein designated VGAM RNA, to host target binding sites on VGAM187 host target RNA, herein designated VGAM HOST TARGET RNA, such as BINDING SITE I, BINDING SITE II and BINDING SITE III, inhibits translation of VGAM187 host target RNA into VGAM187 host target protein, herein designated VGAM HOST TARGET PROTEIN. VGAM host target protein is therefore outlined by a broken line.

It is appreciated that VGAM187 host target gene, herein designated VGAM HOST TARGET GENE, in fact represents a plurality of VGAM187 host target genes. The mRNA of each one of this plurality of VGAM187 host target genes comprises one or more host target binding sites, each having a nucleotide sequence which is at least partly complementary to VGAM187 RNA, herein designated VGAM RNA, and which when bound by VGAM187 RNA causes inhibition of translation of respective one or more VGAM187 host target proteins.

It is further appreciated by one skilled in the art that the mode of translational inhibition illustrated by FIG. 1 with specific reference to translational inhibition exerted by VGAM187 gene, herein designated VGAM GENE, on one or more VGAM187 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM187 is inhibition of expression of host target genes, as part of a novel viral mechanism of attacking a host. Accordingly, utilities of VGAM187 include diagnosis, prevention and treatment of viral infection by Vaccinia Virus. Specific functions, and accordingly utilities, of VGAM187 correlate with, and may be deduced from, the identity of the host target genes which VGAM187 binds and inhibits, and the function of these host target genes, as elaborated hereinbelow.

Nucleotide sequences of the VGAM187 precursor RNA, herein designated VGAM PRECURSOR RNA, and of the ‘diced’ VGAM187 RNA, herein designated VGAM RNA, and a schematic representation of the secondary folding of VGAM187 folded precursor RNA, herein designated VGAM FOLDED PRECURSOR RNA, of VGAM187 are further described hereinbelow with reference to Table 1.

Nucleotide sequences of host target binding sites, such as BINDING SITE-I, BINDING SITE-II and BINDING SITE-III of FIG. 1, found on VGAM187 host target RNA, and schematic representation of the complementarity of each of these host target binding sites to VGAM187 RNA, herein designated VGAM RNA, are described hereinbelow with reference to Table 2.

As mentioned hereinabove with reference to FIG. 1, a function of VGAM187 gene, herein designated VGAM is inhibition of expression of VGAM187 target genes. It is appreciated that specific functions, and accordingly utilities, of VGAM187 correlate with, and may be deduced from, the identity of the target genes which VGAM187 binds and inhibits, and the function of these target genes, as elaborated hereinbelow.

Adenylate Cyclase 2 (brain) (ADCY2, Accession XM_{—}036383) is a VGAM187 host target gene. ADCY2 BINDING SITE is HOST TARGET binding site found in the 3′ untranslated region of mRNA encoded by ADCY2, corresponding to a HOST TARGET binding site such as BINDING SITE I, BINDING SITE II or BINDING SITE III. Table 2 illustrates the complementarity of the nucleotide sequences of ADCY2 BINDING SITE, designated SEQ ID:2710, to the nucleotide sequence of VGAM187 RNA, herein designated VGAM RNA, also designated SEQ ID:522.

A function of VGAM187 is therefore inhibition of Adenylate Cyclase 2 (brain) (ADCY2, Accession XM_{—}036383), a gene which Adenylate cyclase (type 2), an ATP-pyrophosphate lyase; converts ATP to cAMP. Accordingly, utilities of VGAM187 include diagnosis, prevention and treatment of diseases and clinical conditions associated with ADCY2. The function of ADCY2 and its association with various diseases and clinical conditions, has been established by previous studies, as described hereinabove with reference to VGAM49. Cyclin A1 (CCNA1, Accession NM_{—}003914) is another VGAM187 host target gene. CCNA1 BINDING SITE is HOST TARGET binding site found in the 3′ untranslated region of mRNA encoded by CCNA1, corresponding to a HOST TARGET binding site such as BINDING SITE I, BINDING SITE II or BINDING SITE III. Table 2 illustrates the complementarity of the nucleotide sequences of CCNA1 BINDING SITE, designated SEQ ID: 1070, to the nucleotide sequence of VGAM187 RNA, herein designated VGAM RNA, also designated SEQ ID:522.

Another function of VGAM187 is therefore inhibition of Cyclin A1 (CCNA1, Accession NM_{—}003914), a gene which Cyclin A1; acts as a CDK kinase regulatory subunit that interacts with CDK2; strongly similar to murine Ccna1. Accordingly, utilities of VGAM187 include diagnosis, prevention and treatment of diseases and clinical conditions associated with CCNA1. The function of CCNA1 has been established by previous studies. The kinase activity of the CDK2 (OMIM Ref. No. 116953)-cyclin A complex, which peaks at the G1/S transition of the cell cycle, is required for the cell to enter S phase. Cyclin A also forms a complex with CDC2 (OMIM Ref. No. 116940), whose activity peaks at the G2/M transition, and the kinase activity of CDC2-cyclin A is required for the cell to enter M phase. Xenopus and mouse contain 2 cyclin A genes, cyclin A1 and cyclin A2, the homolog of human cyclin A (CCNA2; 123835). By searching an EST database with a cyclin consensus sequence, Yang et al. (1997) identified a cDNA encoding the human cyclin A1. The predicted 465-amino acid protein shares 84%, 59%, and 48% sequence identity with mouse cyclin A1, Xenopus cyclin A1, and human cyclin A, respectively. Northern blot analysis revealed that the 2.1-kb cyclin A1 mRNA was only expressed in testis and brain, with the level of expression in testis being much higher. In addition, the authors detected cyclin A1 mRNA in ML-1 and several other myeloid leukemia cell lines. The expression of cyclin A1 was differentiation- and cell cycle-regulated in ML-1 cells. Cyclin A1 protein had an apparent molecular mass of 65 kD and coimmunoprecipitated with CDK2 and showed histone Hl (see OMIM Ref. No. 142711) kinase activity in ML-1 cell extracts. Muller et al. (1999) reported that the cyclin A1 gene contains 9 exons and spans 13 kb. Cyclin A1 promoter activity depends on 4 GC boxes, and members of the Sp1 (OMIM Ref. No. 189906) family appear to be involved in directing expression of cyclin A1 in both a tissue- and cell cycle-specific manner. Elevated levels of expression of cyclin A1 have been found in several leukemia cell lines and blood samples from patients with hematopoietic malignancies, notably, acute myeloid leukemia. To evaluate whether cyclin A1 is directly involved in the development of myeloid leukemia, Liao et al. (2001) overexpressed mouse cyclin A1 protein in the myeloid lineage of transgenic mice under the direction of the human cathepsin G (CTSG; 116830) promoter. The resulting transgenic mice exhibited an increased proportion of immature myeloid cells in the peripheral blood, bone marrow, and spleen. The abnormal myelopoiesis developed within a few months after birth and progressed to overt acute myeloid leukemia at a frequency of approximately 15% over the course of 7 to 14 months. Both abnormalities in myelopoiesis and the leukemic state could be transplanted to irradiated SCID (severe combined immunodeficient) mice. The observations suggested that cyclin A1 overexpression results in abnormal myelopoiesis and is necessary, but not sufficient, in the cooperative events inducing the transformed phenotype. The data further supported an important role of cyclin A1 in hematopoiesis and the etiology of myeloid leukemia.

Full details of the abovementioned studies are described in the following publications, the disclosure of which are hereby incorporated by reference:
Liao, C.; Wang, X. Y.; Wei, H. Q.; Li, S. Q.; Merghoub, T.; Pandolfi, P. P.; Wolgemuth, D. J.: Altered myelopoiesis and the development of acute myeloid leukemia in transgenic mice overexpressing cyclin A1. Proc. Nat. Acad. Sci. 98: 6853-6858, 2001.; and
Yang, R.; Morosetti, R.; Koeffler, H. P.: Characterization of a second human cyclin A that is highly expressed in testis and in several leukemic cell lines. Cancer Res. 57: 913-920, 19.

Further studies establishing the function and utilities of CCNA1 are found in John Hopkins OMIM database record ID 604036, and in sited publications numbered 1870-187 and 1053 listed in the bibliography section hereinbelow, which are also hereby incorporated by reference. Component of Oligomeric Golgi Complex 6 (COG6, Accession XM_{—}053233) is another VGAM187 host target gene. COG6 BINDING SITE is HOST TARGET binding site found in the 3′ untranslated region of mRNA encoded by COG6, corresponding to a HOST TARGET binding site such as BINDING SITE I, BINDING SITE II or BINDING SITE III. Table 2 illustrates the complementarity of the nucleotide sequences of COG6 BINDING SITE, designated SEQ ID:2961, to the nucleotide sequence of VGAM187 RNA, herein designated VGAM RNA, also designated SEQ ID:522.

Another function of VGAM187 is therefore inhibition of Component of Oligomeric Golgi Complex 6 (COG6, Accession XM_{—}053233), a gene which is critical for the structure and function of the Golgi apparatus. Accordingly, utilities of VGAM187 include diagnosis, prevention and treatment of diseases and clinical conditions associated with COG6. The function of COG6 has been established by previous studies. Multiprotein complexes are key determinants of Golgi apparatus structure and its capacity for intracellular transport and glycoprotein modification. Several complexes have been identified, including the Golgi transport complex (GTC), the LDLC complex, which is involved in glycosylation reactions, and the SEC34 complex, which is involved in vesicular transport. These 3 complexes are identical and have been termed the conserved oligomeric Golgi (COG) complex, which includes COG6 (Ungar et al., 2002). By SDS-PAGE analysis of bovine brain cytosol, Ungar et al. (2002) identified the 8 subunits of the COG complex. Immunofluorescence microscopy demonstrated that COG1 (LDLB; 606973) colocalizes with COG7 (OMIM Ref. No. 606978), as well as with COG3 (OMIM Ref. No. 606975) and COG5 (OMIM Ref. No. 606821), with a Golgi marker in a perinuclear distribution. Immunoprecipitation analysis showed that all COG subunits interact with COG2 (LDLC; 606974). Ungar et al. (2002) concluded that the COG complex is critical for the structure and function of the Golgi apparatus and can influence intracellular membrane trafficking.

Full details of the abovementioned studies are described in the following publications, the disclosure of which are hereby incorporated by reference:
Ungar, D.; Oka, T.; Brittle, E. E.; Vasile, E.; Lupashin, V. V.; Chatterton, J. E.; Heuser, J. E.; Krieger, M.; Waters, M. G.: Characterization of a mammalian Golgi-localized protein complex, COG, that is required for normal Golgi morphology and function. J. Cell Biol. 157: 405-415, 2002.; and
Hirosawa, M.; Nagase, T.; Ishikawa, K.; Kikuno, R.; Nomura, N.; Ohara, O.: Characterization of cDNA clones selected by the GeneMark analysis from size-fractionated cDNA libraries from.

Further studies establishing the function and utilities of COG6 are found in John Hopkins OMIM database record ID 606977, and in sited publications numbered 1684 listed in the bibliography section hereinbelow, which are also hereby incorporated by reference. Dachshund Homolog (Drosophila) (DACH, Accession NM_{—}080759) is another VGAM187 host target gene. DACH BINDING SITE is HOST TARGET binding site found in the 3′ untranslated region of mRNA encoded by DACH, corresponding to a HOST TARGET binding site such as BINDING SITE I, BINDING SITE II or BINDING SITE III. Table 2 illustrates the complementarity of the nucleotide sequences of DACH BINDING SITE, designated SEQ ID:2387, to the nucleotide sequence of VGAM187 RNA, herein designated VGAM RNA, also designated SEQ ID:522.

Another function of VGAM187 is therefore inhibition of Dachshund Homolog (Drosophila) (DACH, Accession NM_{—}080759), a gene which regulates early progenitor cell proliferation during retinogenesis and pituitary development. Accordingly, utilities of VGAM187 include diagnosis, prevention and treatment of diseases and clinical conditions associated with DACH. The function of DACH and its association with various diseases and clinical conditions, has been established by previous studies, as described hereinabove with reference to VGAM170. FK506 Binding Protein 1A, 12 kDa (FKBP1A, Accession NM_{—}000801) is another VGAM187 host target gene. FKBP1A BINDING SITE is HOST TARGET binding site found in the 3′ untranslated region of mRNA encoded by FKBP1A, corresponding to a HOST TARGET binding site such as BINDING SITE I, BINDING SITE II or BINDING SITE III. Table 2 illustrates the complementarity of the nucleotide sequences of FKBP1A BINDING SITE, designated SEQ ID:775, to the nucleotide sequence of VGAM187 RNA, herein designated VGAM RNA, also designated SEQ ID:522.

Another function of VGAM187 is therefore inhibition of FK506 Binding Protein 1A, 12 kDa (FKBP1A, Accession NM_{—}000801), a gene which FK506-binding protein 1A. Accordingly, utilities of VGAM187 include diagnosis, prevention and treatment of diseases and clinical conditions associated with FKBP1A. The function of FKBP1A has been established by previous studies. FK506, a macrolide, is a powerful immunosuppressant like cyclosporin A (CsA). CsA and FK506 are chemically distinct but have remarkably similar immunosuppressive action, although FK506 is many times more potent than CsA. The action of CsA may be mediated through cyclophilin (OMIM Ref. No. 123840), which is identical to peptidyl-prolyl cis-trans isomerase (OMIM Ref. No. PPlase), and the immunosuppressive action of CsA in T cells may be mediated through inhibition of this enzyme activity. FK506-binding protein (FKBP) also has peptidyl-prolyl cis-trans isomerase enzymatic activity; however, whereas cyclophilin binds to, and is inhibited by, CsA but does not recognize FK506, the converse holds for FKBP. Since the 2 drugs have essentially equivalent action on T lymphocytes, it may be that they act through distinct pathways but their mode of action converges on PPlase activities. Maki et al. (1990) isolated and sequenced DNA coding for FKBP from human peripheral blood T lymphocytes by using mixed 20-mer oligonucleotide probes synthesized on the basis of the sequence of bovine FKBP. They found an open reading frame encoding 108 amino acid residues, the first 40 of which were identical to those of the bovine sequence. Analysis showed no significant sequence similarity to any other known protein, including cyclophilin. Southern blot analysis of human genomic DNA digested with different restriction enzymes suggested the existence of only a few copies of the FKBP gene. This is in contrast to the results indicating as many as 20 copies of the cyclophilin gene as well as possible pseudogenes in the mammalian genome. Standaert et al. (1990) likewise isolated a cDNA for FKBP and reported the derived amino acid sequence. The human FKBP cDNA sequence showed significant similarity to an open reading frame in the genome of Neisseria meningitidis. Wang et al. (1994) reported that in a yeast genetic screen, FKBP1 interacted with various type I receptors, including the TGF-beta type I receptor (OMIM Ref. No. 190181). Deletion, point mutation, and co-immunoprecipitation studies demonstrated the specificity of this interaction, and competitive binding assays indicated that the type I receptor may be a natural ligand for FKBP1. Wang et al. (1994) concluded that FKBP1 may play a role in type I receptor-mediated signaling. Peattie et al. (1994) identified 3 distinct mRNAs for FKBP12, designated A, B, and C, that result from differential splicing or polyadenylation. All 3 encode the same protein sequence. The ryanodine receptor on the sarcoplasmic reticulum is the major source of calcium required for cardiac muscle excitation-contraction coupling. The channel is a tetramer comprised of 4 RYR2 (OMIM Ref. No. 180902) polypeptides and 4 FK506-binding proteins (OMIM Ref. No. FKBP1A). Marx et al. (2000) showed that protein kinase A (PKA; OMIM Ref. No. 176911) phosphorylation of RYR2 dissociates FKBP1A and regulates the channel open probability. Using cosedimentation and coimmunoprecipitation, the authors defined a macromolecular complex comprised of RYR2, FKBP1A, PKA, the protein phosphatases PP1 (see OMIM Ref. No. 603771) and PP2A (see OMIM Ref. No. 603113), and an anchoring protein, AKAP6 (OMIM Ref. No. 604691). In failing human hearts, Marx et al. (2000) showed that RYR2 is PKA hyperphosphorylated, resulting in defective channel function due to increased sensitivity to calcium-induced activation. To define the functions of FKBP12 in vivo, Shou et al. (1998) generated mutant mice deficient in FKBP12 using embryonic stem (ES) cell technology. FKBP12-deficient mice had normal skeletal muscle but had severe dilated cardiomyopathy and ventricular septal defects that mimicked a human congenital heart disorder, namely noncompaction of left ventricular myocardium (see OMIM Ref. No. 300183 for an X-linked form of myocardial noncompaction). About 9% of the mutants exhibited exencephaly secondary to a defect in neural tube closure. Physiologic studies demonstrated that FKBP12 is dispensable for TGF-beta-mediated signaling, but modulates the calcium release activity of both skeletal and cardiac ryanodine receptors.

Full details of the abovementioned studies are described in the following publications, the disclosure of which are hereby incorporated by reference:
Standaert, R. F.; Galat, A.; Verdine, G. L.; Schreiber, S. L.: Molecular cloning and overexpression of the human FK506-binding protein FKBP. Nature 346: 671-674, 1990.; and
Shou, W.; Aghdasi, B.; Armstrong, D. L.; Guo, Q.; Bao, S.; Charng, M.-J.; Mathews, L. M.; Schneider, M. D.; Hamilton, S. L.; Matzuk, M. M.: Cardiac defects and altered ryanodine recepto.

Further studies establishing the function and utilities of FKBP1A are found in John Hopkins OMIM database record ID 186945, and in sited publications numbered 2271-227 and 2361-2279 listed in the bibliography section hereinbelow, which are also hereby incorporated by reference. Heat Shock 60 kDa Protein 1 (chaperonin) (HSPD1, Accession XM_{—}012182) is another VGAM187 host target gene. HSPD1 BINDING SITE is HOST TARGET binding site found in the 3′ untranslated region of mRNA encoded by HSPD1, corresponding to a HOST TARGET binding site such as BINDING SITE I, BINDING SITE II or BINDING SITE III. Table 2 illustrates the complementarity of the nucleotide sequences of HSPD1 BINDING SITE, designated SEQ ID:2559, to the nucleotide sequence of VGAM187 RNA, herein designated VGAM RNA, also designated SEQ ID:522.

Another function of VGAM187 is therefore inhibition of Heat Shock 60 kDa Protein 1 (chaperonin) (HSPD1, Accession XM_{—}012182), a gene which is implicated in mitochondrial protein import and macromolecular assembly. may facilitate the correct folding of imported proteins. may also prevent misfolding and promote the refolding and proper assembly of unfolded polypeptides generated under stress conditions in the mitochondrial matrix. Accordingly, utilities of VGAM187 include diagnosis, prevention and treatment of diseases and clinical conditions associated with HSPD1. The function of HSPD1 and its association with various diseases and clinical conditions, has been established by previous studies, as described hereinabove with reference to VGAM130. JJAZ1 (Accession NM_{—}015355) is another VGAM187 host target gene. JJAZ1 BINDING SITE is HOST TARGET binding site found in the 3′ untranslated region of mRNA encoded by JJAZ1, corresponding to a HOST TARGET binding site such as BINDING SITE I, BINDING SITE II or BINDING SITE III. Table 2 illustrates the complementarity of the nucleotide sequences of JJAZ1 BINDING SITE, designated SEQ ID: 1622, to the nucleotide sequence of VGAM187 RNA, herein designated VGAM RNA, also designated SEQ ID:522.

Another function of VGAM187 is therefore inhibition of JJAZ1 (Accession NM_{—}015355), a gene which is a zinc finger protein. Accordingly, utilities of VGAM187 include diagnosis, prevention and treatment of diseases and clinical conditions associated with JJAZ1. The function of JJAZ1 has been established by previous studies. Nagase et al. (1995) deduced the sequence of a full-length cDNA clone from cell line KG-1, which they designated KIAA0160, encoding a predicted 803-amino acid protein. Northern blot analysis revealed expression in all tissues tested. A variety of cytogenetic abnormalities involving chromosome 7 have been reported in endometrial stromal sarcomas, including a recurrent t(7;17)(p15;q21). Koontz et al. (2001) identified 2 zinc finger genes, which they termed JAZF1 (OMIM Ref. No. 606246) and JJAZ1, at the sites of the 7p15 and 17q21 breakpoints, respectively. Analyses of tumor RNA indicated that a JAZF1/JJAZ1 fusion was present in all types of endometrial stromal tumors; however, the fusion appeared to be rarer among endometrial stromal sarcomas that would be considered high-grade according to certain classification schemes

Full details of the abovementioned studies are described in the following publications, the disclosure of which are hereby incorporated by reference:
Koontz, J. I.; Soreng, A. L.; Nucci, M.; Kuo, F. C.; Pauwels, P.; van den Berghe, H.; Cin, P. D.; Fletcher, J. A.; Sklar, J.: Frequent fusion of the JAZF1 and JJAZ1 genes in endometrial stromal tumors. Proc. Nat. Acad. Sci. 98: 6348-6353, 2001.; and
Nagase, T.; Seki, N.; Tanaka, A.; Ishikawa, K.; Nomura, N.: Prediction of the coding sequences of unidentified human genes. IV. The coding sequences of 40 new genes (KIAA0121-KIAA0160).

Further studies establishing the function and utilities of JJAZ1 are found in John Hopkins OMIM database record ID 606245, and in sited publications numbered 141 and 2524 listed in the bibliography section hereinbelow, which are also hereby incorporated by reference. Mannosidase, Alpha, Class 1A, Member 1 (MAN1A1, Accession XM_{—}166312) is another VGAM187 host target gene. MAN1A1 BINDING SITE is HOST TARGET binding site found in the 3′ untranslated region of mRNA encoded by MAN1A1, corresponding to a HOST TARGET binding site such as BINDING SITE I, BINDING SITE II or BINDING SITE III. Table 2 illustrates the complementarity of the nucleotide sequences of MAN1A1 BINDING SITE, designated SEQ ID:3543, to the nucleotide sequence of VGAM187 RNA, herein designated VGAM RNA, also designated SEQ ID:522.

Another function of VGAM187 is therefore inhibition of Mannosidase, Alpha, Class 1A, Member 1 (MAN1A1, Accession XM_{—}166312), a gene which removes 3 distinct mannose residues from peptide-bound Man(9)-GlcNAc(2) oligosaccharides. Accordingly, utilities of VGAM187 include diagnosis, prevention and treatment of diseases and clinical conditions associated with MAN1A1. The function of MAN1A1 has been established by previous studies. Man(9)-mannosidase (alpha-1,2-mannosidase 1A) catalyzes the removal of 3 distinct mannose residues from peptide-bound Man(9)-GlcNAc(2) oligosaccharides. See MAN2A1 (OMIM Ref. No. 154582) for general information. Using an oligonucleotide probe derived from a pig liver Man(9)-mannosidase-specific cDNA template, Bause et al. (1993) isolated Man(9)-mannosidase from a human kidney cDNA library. The full-length cDNA predicted a 625-amino acid protein with a calculated molecular mass of 71 kD. Man(9)-mannosidase is a type II transmembrane protein with a short cytoplasmic polypeptide tail, a single transmembrane domain acting as a noncleavable signal sequence, a large luminal catalytic domain, and 3 potential N-glycosylation sites

Full details of the abovementioned studies are described in the following publications, the disclosure of which are hereby incorporated by reference:
Bause, E.; Bieberich, E.; Rolfs, A.; Volker, C.; Schmidt, B.: Molecular cloning and primary structure of Man(9)-mannosidase from human kidney. Eur. J. Biochem. 217: 535-540, 1993.; and
Tremblay, L. 0; Campbell Dyke, N.; Herscovics, A.: Molecular cloning, chromosomal mapping and tissue-specific expression of a novel human alpha-1,2-mannosidase gene involved in N-glycan.

Further studies establishing the function and utilities of MAN1A1 are found in John Hopkins OMIM database record ID 604344, and in sited publications numbered 1129-1130 listed in the bibliography section hereinbelow, which are also hereby incorporated by reference. Protein Kinase (cAMP-dependent, catalytic) Inhibitor Beta (PKIB, Accession NM_{—}032471) is another VGAM187 host target gene. PKIB BINDING SITE is HOST TARGET binding site found in the 3′ untranslated region of mRNA encoded by PKIB, corresponding to a HOST TARGET binding site such as BINDING SITE I, BINDING SITE II or BINDING SITE III. Table 2 illustrates the complementarity of the nucleotide sequences of PKIB BINDING SITE, designated SEQ ID:2254, to the nucleotide sequence of VGAM187 RNA, herein designated VGAM RNA, also designated SEQ ID:522.

Another function of VGAM187 is therefore inhibition of Protein Kinase (cAMP-dependent, catalytic) Inhibitor Beta (PKIB, Accession NM_{—}032471). Accordingly, utilities of VGAM187 include diagnosis, prevention and treatment of diseases and clinical conditions associated with PKIB. Ribonucleotide Reductase M2 B (TP53 inducible) (RRM2B, Accession XM_{—}042096) is another VGAM187 host target gene. RRM2B BINDING SITE is HOST TARGET binding site found in the 3′ untranslated region of mRNA encoded by RRM2B, corresponding to a HOST TARGET binding site such as BINDING SITE I, BINDING SITE II or BINDING SITE III. Table 2 illustrates the complementarity of the nucleotide sequences of RRM2B BINDING SITE, designated SEQ ID:2799, to the nucleotide sequence of VGAM187 RNA, herein designated VGAM RNA, also designated SEQ ID:522.

Another function of VGAM187 is therefore inhibition of Ribonucleotide Reductase M2 B (TP53 inducible) (RRM2B, Accession XM_{—}042096). Accordingly, utilities of VGAM187 include diagnosis, prevention and treatment of diseases and clinical conditions associated with RRM2B. SWAP70 (Accession XM_{—}049197) is another VGAM187 host target gene. SWAP70 BINDING SITE is HOST TARGET binding site found in the 3′ untranslated region of mRNA encoded by SWAP70, corresponding to a HOST TARGET binding site such as BINDING SITE I, BINDING SITE II or BINDING SITE III. Table 2 illustrates the complementarity of the nucleotide sequences of SWAP70 BINDING SITE, designated SEQ ID:2917, to the nucleotide sequence of VGAM187 RNA, herein designated VGAM RNA, also designated SEQ ID:522.

Another function of VGAM187 is therefore inhibition of SWAP70 (Accession XM_{—}049197), a gene which is involved not only in nuclear events but also in signaling in B-cell activation. Accordingly, utilities of VGAM187 include diagnosis, prevention and treatment of diseases and clinical conditions associated with SWAP70. The function of SWAP70 and its association with various diseases and clinical conditions, has been established by previous studies, as described hereinabove with reference to VGAM75. Abhydrolase Domain Containing 3 (ABHD3, Accession NM_{—}138340) is another VGAM187 host target gene. ABHD3 BINDING SITE is HOST TARGET binding site found in the 3′ untranslated region of mRNA encoded by ABHD3, corresponding to a HOST TARGET binding site such as BINDING SITE I, BINDING SITE II or BINDING SITE III. Table 2 illustrates the complementarity of the nucleotide sequences of ABHD3 BINDING SITE, designated SEQ ID:2436, to the nucleotide sequence of VGAM187 RNA, herein designated VGAM RNA, also designated SEQ ID:522.

Another function of VGAM187 is therefore inhibition of Abhydrolase Domain Containing 3 (ABHD3, Accession NM_{—}138340). Accordingly, utilities of VGAM187 include diagnosis, prevention and treatment of diseases and clinical conditions associated with ABHD3. ATIP1 (Accession NM_{—}020749) is another VGAM187 host target gene. ATIP1 BINDING SITE is HOST TARGET binding site found in the 3′ untranslated region of mRNA encoded by ATIP1, corresponding to a HOST TARGET binding site such as BINDING SITE I, BINDING SITE II or BINDING SITE III. Table 2 illustrates the complementarity of the nucleotide sequences of ATIP1 BINDING SITE, designated SEQ ID:1924, to the nucleotide sequence of VGAM187 RNA, herein designated VGAM RNA, also designated SEQ ID:522.

Another function of VGAM187 is therefore inhibition of ATIP1 (Accession NM_{—}020749). Accordingly, utilities of VGAM187 include diagnosis, prevention and treatment of diseases and clinical conditions associated with ATIP1. DKFZP564F013 (Accession XM_{—}168479) is another VGAM187 host target gene. DKFZP564F013 BINDING SITE is HOST TARGET binding site found in the 3′ untranslated region of mRNA encoded by DKFZP564F013, corresponding to a HOST TARGET binding site such as BINDING SITE I, BINDING SITE II or BINDING SITE III. Table 2 illustrates the complementarity of the nucleotide sequences of DKFZP564F013 BINDING SITE, designated SEQ ID:3640, to the nucleotide sequence of VGAM187 RNA, herein designated VGAM RNA, also designated SEQ ID:522.

Another function of VGAM187 is therefore inhibition of DKFZP564F013 (Accession XM_{—}168479). Accordingly, utilities of VGAM187 include diagnosis, prevention and treatment of diseases and clinical conditions associated with DKFZP564F013. FN5 (Accession NM_{—}020179) is another VGAM187 host target gene. FN5 BINDING SITE is HOST TARGET binding site found in the 3′ untranslated region of mRNA encoded by FN5, corresponding to a HOST TARGET binding site such as BINDING SITE I, BINDING SITE II or BINDING SITE III. Table 2 illustrates the complementarity of the nucleotide sequences of FN5 BINDING SITE, designated SEQ ID:1898, to the nucleotide sequence of VGAM187 RNA, herein designated VGAM RNA, also designated SEQ ID:522.

Another function of VGAM187 is therefore inhibition of FN5 (Accession NM_{—}020179). Accordingly, utilities of VGAM187 include diagnosis, prevention and treatment of diseases and clinical conditions associated with FN5. KIAA0416 (Accession NM_{—}015564) is another VGAM187 host target gene. KIAA0416 BINDING SITE is HOST TARGET binding site found in the 3′ untranslated region of mRNA encoded by KIAA0416, corresponding to a HOST TARGET binding site such as BINDING SITE I, BINDING SITE II or BINDING SITE III. Table 2 illustrates the complementarity of the nucleotide sequences of KIAA0416 BINDING SITE, designated SEQ ID: 1637, to the nucleotide sequence of VGAM187 RNA, herein designated VGAM RNA, also designated SEQ ID:522.

Another function of VGAM187 is therefore inhibition of KIAA0416 (Accession NM_{—}015564). Accordingly, utilities of VGAM187 include diagnosis, prevention and treatment of diseases and clinical conditions associated with KIAA0416. KIAA0455 (Accession XM_{—}051785) is another VGAM187 host target gene. KIAA0455 BINDING SITE is HOST TARGET binding site found in the 3′ untranslated region of mRNA encoded by KIAA0455, corresponding to a HOST TARGET binding site such as BINDING SITE I, BINDING SITE II or BINDING SITE III. Table 2 illustrates the complementarity of the nucleotide sequences of KIAA0455 BINDING SITE, designated SEQ ID:2948, to the nucleotide sequence of VGAM187 RNA, herein designated VGAM RNA, also designated SEQ ID:522.

Another function of VGAM187 is therefore inhibition of KIAA0455 (Accession XM_{—}051785). Accordingly, utilities of VGAM187 include diagnosis, prevention and treatment of diseases and clinical conditions associated with KIAA0455. KIAA0912 (Accession XM_{—}034904) is another VGAM187 host target gene. KIAA0912 BINDING SITE is HOST TARGET binding site found in the 3′ untranslated region of mRNA encoded by KIAA0912, corresponding to a HOST TARGET binding site such as BINDING SITE I, BINDING SITE II or BINDING SITE III. Table 2 illustrates the complementarity of the nucleotide sequences of KIAA0912 BINDING SITE, designated SEQ ID:2689, to the nucleotide sequence of VGAM187 RNA, herein designated VGAM RNA, also designated SEQ ID:522.

Another function of VGAM187 is therefore inhibition of KIAA0912 (Accession XM_{—}034904). Accordingly, utilities of VGAM187 include diagnosis, prevention and treatment of diseases and clinical conditions associated with KIAA0912. KIAA0981 (Accession XM_{—}028867) is another VGAM187 host target gene. KIAA0981 BINDING SITE is HOST TARGET binding site found in the 3′ untranslated region of mRNA encoded by KIAA0981, corresponding to a HOST TARGET binding site such as BINDING SITE I, BINDING SITE II or BINDING SITE III. Table 2 illustrates the complementarity of the nucleotide sequences of KIAA0981 BINDING SITE, designated SEQ ID:2597, to the nucleotide sequence of VGAM187 RNA, herein designated VGAM RNA, also designated SEQ ID:522.

Another function of VGAM187 is therefore inhibition of KIAA0981 (Accession XM_{—}028867). Accordingly, utilities of VGAM187 include diagnosis, prevention and treatment of diseases and clinical conditions associated with KIAA0981. KIAA1911 (Accession XM_{—}056302) is another VGAM187 host target gene. KIAA1911 BINDING SITE is HOST TARGET binding site found in the 3′ untranslated region of mRNA encoded by KIAA1911, corresponding to a HOST TARGET binding site such as BINDING SITE I, BINDING SITE II or BINDING SITE III. Table 2 illustrates the complementarity of the nucleotide sequences of KIAA1911 BINDING SITE, designated SEQ ID:2976, to the nucleotide sequence of VGAM187 RNA, herein designated VGAM RNA, also designated SEQ ID:522.

Another function of VGAM187 is therefore inhibition of KIAA1911 (Accession XM_{—}056302). Accordingly, utilities of VGAM187 include diagnosis, prevention and treatment of diseases and clinical conditions associated with KIAA1911. Keratin, Hair, Basic, 2 (KRTHB2, Accession NM_{—}033033) is another VGAM187 host target gene. KRTHB2 BINDING SITE is HOST TARGET binding site found in the 3′ untranslated region of mRNA encoded by KRTHB2, corresponding to a HOST TARGET binding site such as BINDING SITE I, BINDING SITE II or BINDING SITE III. Table 2 illustrates the complementarity of the nucleotide sequences of KRTHB2 BINDING SITE, designated SEQ ID:2310, to the nucleotide sequence of VGAM187 RNA, herein designated VGAM RNA, also designated SEQ ID:522.

Another function of VGAM187 is therefore inhibition of Keratin, Hair, Basic, 2 (KRTHB2, Accession NM_{—}033033). Accordingly, utilities of VGAM187 include diagnosis, prevention and treatment of diseases and clinical conditions associated with KRTHB2. Ribosomal Protein S6 Kinase, 52 kDa, Polypeptide 1 (RPS6KC1, Accession NM_{—}012424) is another VGAM187 host target gene. RPS6KC1 BINDING SITE is HOST TARGET binding site found in the 3′ untranslated region of mRNA encoded by RPS6KC1, corresponding to a HOST TARGET binding site such as BINDING SITE I, BINDING SITE II or BINDING SITE III. Table 2 illustrates the complementarity of the nucleotide sequences of RPS6KC1 BINDING SITE, designated SEQ ID: 1429, to the nucleotide sequence of VGAM187 RNA, herein designated VGAM RNA, also designated SEQ ID:522.

Another function of VGAM187 is therefore inhibition of Ribosomal Protein S6 Kinase, 52 kDa, Polypeptide 1 (RPS6KC1, Accession NM_{—}012424). Accordingly, utilities of VGAM187 include diagnosis, prevention and treatment of diseases and clinical conditions associated with RPS6KC1. Translocase of Outer Mitochondrial Membrane 70 Homolog A (yeast) (TOMM70A, Accession NM_{—}014820) is another VGAM187 host target gene. TOMM70A BINDING SITE is HOST TARGET binding site found in the 3′ untranslated region of mRNA encoded by TOMM70A, corresponding to a HOST TARGET binding site such as BINDING SITE I, BINDING SITE II or BINDING SITE III. Table 2 illustrates the complementarity of the nucleotide sequences of TOMM70A BINDING SITE, designated SEQ ID:1558, to the nucleotide sequence of VGAM187 RNA, herein designated VGAM RNA, also designated SEQ ID:522.

Another function of VGAM187 is therefore inhibition of Translocase of Outer Mitochondrial Membrane 70 Homolog A (yeast) (TOMM70A, Accession NM_{—}014820). Accordingly, utilities of VGAM187 include diagnosis, prevention and treatment of diseases and clinical conditions associated with TOMM70A. FIG. 1 further provides a conceptual description of a novel bioinformatically detected viral gene of the present invention, referred to here as Viral Genomic Address Messenger 188 (VGAM188) viral gene, which modulates expression of respective host target genes thereof, the function and utility of which host target genes is known in the art.

VGAM188 is a novel bioinformatically detected regulatory, non protein coding, viral micro RNA (miRNA) gene. The method by which VGAM188 was detected is described hereinabove with reference to FIGS. 1-8.

VGAM188 gene, herein designated VGAM GENE, is a viral gene contained in the genome of Vaccinia Virus. VGAM188 host target gene, herein designated VGAM HOST TARGET GENE, is a human gene contained in the human genome.

VGAM188 gene encodes a VGAM188 precursor RNA, herein designated VGAM PRECURSOR RNA. Similar to other miRNA genes, and unlike most ordinary genes, VGAM188 precursor RNA does not encode a protein. A nucleotide sequence identical or highly similar to the nucleotide sequence of VGAM188 precursor RNA is designated SEQ ID:174, and is provided hereinbelow with reference to the sequence listing part. Nucleotide sequence SEQ ID:174 is located at position 97335 relative to the genome of Vaccinia Virus.

VGAM188 precursor RNA folds onto itself, forming VGAM188 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM188 folded precursor RNA into VGAM188 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM188 RNA is designated SEQ ID:523, and is provided hereinbelow with reference to the sequence listing part.

VGAM188 host target gene, herein designated VGAM HOST TARGET GENE, encodes a corresponding messenger RNA, VGAM188 host target RNA, herein designated VGAM HOST TARGET RNA. VGAM188 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM188 RNA, herein designated VGAM RNA, binds complementarily to one or more host target binding sites located in untranslated regions of VGAM188 host target RNA, herein designated VGAM HOST TARGET RNA. This complementary binding is due to the fact that the nucleotide sequence of VGAM188 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM188 RNA, herein designated VGAM RNA, may have a different number of host target binding sites in untranslated regions of a VGAM188 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM188 RNA, herein designated VGAM RNA, to host target binding sites on VGAM188 host target RNA, herein designated VGAM HOST TARGET RNA, such as BINDING SITE I, BINDING SITE II and BINDING SITE III, inhibits translation of VGAM188 host target RNA into VGAM188 host target protein, herein designated VGAM HOST TARGET PROTEIN. VGAM host target protein is therefore outlined by a broken line.

It is appreciated that VGAM188 host target gene, herein designated VGAM HOST TARGET GENE, in fact represents a plurality of VGAM188 host target genes. The mRNA of each one of this plurality of VGAM188 host target genes comprises one or more host target binding sites, each having a nucleotide sequence which is at least partly complementary to VGAM188 RNA, herein designated VGAM RNA, and which when bound by VGAM188 RNA causes inhibition of translation of respective one or more VGAM188 host target proteins.

It is further appreciated by one skilled in the art that the mode of translational inhibition illustrated by FIG. 1 with specific reference to translational inhibition exerted by VGAM188 gene, herein designated VGAM GENE, on one or more VGAM188 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM188 is inhibition of expression of host target genes, as part of a novel viral mechanism of attacking a host. Accordingly, utilities of VGAM188 include diagnosis, prevention and treatment of viral infection by Vaccinia Virus. Specific functions, and accordingly utilities, of VGAM188 correlate with, and may be deduced from, the identity of the host target genes which VGAM188 binds and inhibits, and the function of these host target genes, as elaborated hereinbelow.

Nucleotide sequences of the VGAM188 precursor RNA, herein designated VGAM PRECURSOR RNA, and of the ‘diced’ VGAM188 RNA, herein designated VGAM RNA, and a schematic representation of the secondary folding of VGAM188 folded precursor RNA, herein designated VGAM FOLDED PRECURSOR RNA, of VGAM188 are further described hereinbelow with reference to Table 1.

Nucleotide sequences of host target binding sites, such as BINDING SITE-I, BINDING SITE-II and BINDING SITE-III of FIG. 1, found on VGAM188 host target RNA, and schematic representation of the complementarity of each of these host target binding sites to VGAM188 RNA, herein designated VGAM RNA, are described hereinbelow with reference to Table 2.

As mentioned hereinabove with reference to FIG. 1, a function of VGAM188 gene, herein designated VGAM is inhibition of expression of VGAM188 target genes. It is appreciated that specific functions, and accordingly utilities, of VGAM188 correlate with, and may be deduced from, the identity of the target genes which VGAM188 binds and inhibits, and the function of these target genes, as elaborated hereinbelow.

CDC23 (cell division cycle 23, yeast, homolog) (CDC23, Accession NM_{—}004661) is a VGAM188 host target gene. CDC23 BINDING SITE is HOST TARGET binding site found in the 3′ untranslated region of mRNA encoded by CDC23, corresponding to a HOST TARGET binding site such as BINDING SITE I, BINDING SITE II or BINDING SITE III. Table 2 illustrates the complementarity of the nucleotide sequences of CDC23 BINDING SITE, designated SEQ ID:1141, to the nucleotide sequence of VGAM188 RNA, herein designated VGAM RNA, also designated SEQ ID:523.

A function of VGAM188 is therefore inhibition of CDC23 (cell division cycle 23, yeast, homolog) (CDC23, Accession NM_{—}004661), a gene which is the cell cycle-regulated component of the mitotic cyclin degradation system. Accordingly, utilities of VGAM188 include diagnosis, prevention and treatment of diseases and clinical conditions associated with CDC23. The function of CDC23 has been established by previous studies. is the cell cycle-regulated component of the mitotic cyclin degradation system.

Full details of the abovementioned studies are described in the following publications, the disclosure of which are hereby incorporated by reference:
Yu, H.; Peters, J.-M.; King, R. W.; Page, A. M.; Hieter, P.; Kirschner, M. W.: Identification of a cullin homology region in a subunit of the anaphase-promoting complex. Science 279: 1219-1222, 1998.; and
Zhao, N.; Lai, F.; Fernald, A. A.; Eisenbart, J. D.; Espinosa, R., III.; Wang, P. W.; Le Beau, M. M.: Human CDC23: cDNA cloning, mapping to 5q31, genomic structure, and evaluation as a.

Further studies establishing the function and utilities of CDC23 are found in John Hopkins OMIM database record ID 603462, and in sited publications numbered 661-662 listed in the bibliography section hereinbelow, which are also hereby incorporated by reference. Egl Nine Homolog 3 (C. elegans) (EGLN3, Accession NM_{—}033344) is another VGAM188 host target gene. EGLN3 BINDING SITE1 and EGLN3 BINDING SITE2 are HOST TARGET binding sites found in untranslated regions of mRNA encoded by EGLN3, corresponding to HOST TARGET binding sites such as BINDING SITE I, BINDING SITE II or BINDING SITE III. Table 2 illustrates the complementarity of the nucleotide sequences of EGLN3 BINDING SITE1 and EGLN3 BINDING SITE2, designated SEQ ID:2330 and SEQ ID:1976 respectively, to the nucleotide sequence of VGAM188 RNA, herein designated VGAM RNA, also designated SEQ ID:523.

Another function of VGAM188 is therefore inhibition of Egl Nine Homolog 3 (C. elegans) (EGLN3, Accession NM_{—}033344), a gene which is an essential component of the pathway. Accordingly, utilities of VGAM188 include diagnosis, prevention and treatment of diseases and clinical conditions associated with EGLN3. The function of EGLN3 has been established by previous studies. In cultured mammalian cells, Bruick and McKnight (2001) found that the inappropriate accumulation of HIF caused by forced expression of the HIF1-alpha subunit under normoxic conditions was attenuated by coexpression of HPH. Suppression of HPH in cultured Drosophila melanogaster cells by RNA interference resulted in elevated expression of the hypoxia-inducible gene LDH (see OMIM Ref. No. 150000) under normoxic conditions. Bruick and McKnight (2001) concluded that HPH is an essential component of the pathway through which cells sense oxygen. HIF is a transcriptional complex that plays a central role in mammalian oxygen homeostasis. Posttranslational modification by prolyl hydroxylation is a key regulatory event that targets HIF-alpha (HIF1; 603348) subunits for proteasomal destruction via the von Hippel-Lindau (VHL; 193300) ubiquitylation complex. Epstein et al. (2001) defined a conserved HIF-VHL-prolyl hydroxylase pathway in C. elegans and identified Egl9 as a dioxygenase that regulates HIF by prolyl hydroxylation. In mammalian cells, they showed that the HIF-prolyl hydroxylases are represented by 3 proteins with a conserved 2-histidine-1-carboxylate iron coordination motif at the catalytic site. The genes encoding these proteins were cloned and termed PHD1 (OMIM Ref. No. 606424), PHD2 (OMIM Ref. No. 606425), and PHD3 by the authors. Direct modulation of recombinant enzyme activity by graded hypoxia, iron chelation, and cobaltous ions mirrored the characteristics of HIF induction in vivo, fulfilling requirements for these enzymes being oxygen sensors that regulate HIF.

Full details of the abovementioned studies are described in the following publications, the disclosure of which are hereby incorporated by reference:
Bruick, R. K.; McKnight, S. L.: A conserved family of prolyl-4-hydroxylases that modify HIF. Science 294: 1337-1340, 2001.; and
Epstein, A. C. R.; Gleadle, J. M.; McNeill, L. A.; Hewitson, K. S.; O'Rourke, J.; Mole, D. R.; Mukherji, M.; Metzen, E.; Wilson, M. I.; Dhanda, A.; Tian, Y.-M.; Masson, N.; Hamilton, D.

Further studies establishing the function and utilities of EGLN3 are found in John Hopkins OMIM database record ID 606426, and in sited publications numbered 1016-1017 listed in the bibliography section hereinbelow, which are also hereby incorporated by reference. Ring Finger Protein 7 (RNF7, Accession NM_{—}014245) is another VGAM188 host target gene. RNF7 BINDING SITE is HOST TARGET binding site found in the 3′ untranslated region of mRNA encoded by RNF7, corresponding to a HOST TARGET binding site such as BINDING SITE I, BINDING SITE II or BINDING SITE III. Table 2 illustrates the complementarity of the nucleotide sequences of RNF7 BINDING SITE, designated SEQ ID: 1485, to the nucleotide sequence of VGAM188 RNA, herein designated VGAM RNA, also designated SEQ ID:523.

Another function of VGAM188 is therefore inhibition of Ring Finger Protein 7 (RNF7, Accession NM_{—}014245). Accordingly, utilities of VGAM188 include diagnosis, prevention and treatment of diseases and clinical conditions associated with RNF7. Williams-Beuren Syndrome Chromosome Region 5 (WBSCR5, Accession NM_{—}022040) is another VGAM188 host target gene. WBSCR5 BINDING SITE1 through WBSCR5 BINDING SITE3 are HOST TARGET binding sites found in untranslated regions of mRNA encoded by WBSCR5, corresponding to HOST TARGET binding sites such as BINDING SITE I, BINDING SITE II or BINDING SITE III. Table 2 illustrates the complementarity of the nucleotide sequences of WBSCR5 BINDING SITE1 through WBSCR5 BINDING SITE3, designated SEQ ID:1973, SEQ ID:2250 and SEQ ID: 1479 respectively, to the nucleotide sequence of VGAM188 RNA, herein designated VGAM RNA, also designated SEQ ID:523.

Another function of VGAM188 is therefore inhibition of Williams-Beuren Syndrome Chromosome Region 5 (WBSCR5, Accession NM_{—}022040). Accordingly, utilities of VGAM188 include diagnosis, prevention and treatment of diseases and clinical conditions associated with WBSCR5. CDT6 (Accession NM_{—}021146) is another VGAM188 host target gene. CDT6 BINDING SITE is HOST TARGET binding site found in the 3′ untranslated region of mRNA encoded by CDT6, corresponding to a HOST TARGET binding site such as BINDING SITE I, BINDING SITE II or BINDING SITE III. Table 2 illustrates the complementarity of the nucleotide sequences of CDT6 BINDING SITE, designated SEQ ID:1945, to the nucleotide sequence of VGAM188 RNA, herein designated VGAM RNA, also designated SEQ ID:523.

Another function of VGAM188 is therefore inhibition of CDT6 (Accession NM_{—}021146). Accordingly, utilities of VGAM188 include diagnosis, prevention and treatment of diseases and clinical conditions associated with CDT6. DKFZp566D234 (Accession XM_{—}030162) is another VGAM188 host target gene. DKFZp566D234 BINDING SITE is HOST TARGET binding site found in the 3′ untranslated region of mRNA encoded by DKFZp566D234, corresponding to a HOST TARGET binding site such as BINDING SITE I, BINDING SITE II or BINDING SITE III. Table 2 illustrates the complementarity of the nucleotide sequences of DKFZp566D234 BINDING SITE, designated SEQ ID:2613, to the nucleotide sequence of VGAM188 RNA, herein designated VGAM RNA, also designated SEQ ID:523.

Another function of VGAM188 is therefore inhibition of DKFZp566D234 (Accession XM_{—}030162). Accordingly, utilities of VGAM188 include diagnosis, prevention and treatment of diseases and clinical conditions associated with DKFZp566D234. FLJ10525 (Accession NM_{—}018126) is another VGAM188 host target gene. FLJ10525 BINDING SITE is HOST TARGET binding site found in the 3′ untranslated region of mRNA encoded by FLJ10525, corresponding to a HOST TARGET binding site such as BINDING SITE I, BINDING SITE II or BINDING SITE III. Table 2 illustrates the complementarity of the nucleotide sequences of FLJ10525 BINDING SITE, designated SEQ ID:1786, to the nucleotide sequence of VGAM188 RNA, herein designated VGAM RNA, also designated SEQ ID:523.

Another function of VGAM188 is therefore inhibition of FLJ10525 (Accession NM_{—}018126). Accordingly, utilities of VGAM188 include diagnosis, prevention and treatment of diseases and clinical conditions associated with FLJ10525. FLJ12078 (Accession NM_{—}024977) is another VGAM188 host target gene. FLJ12078 BINDING SITE is HOST TARGET binding site found in the 5′ untranslated region of mRNA encoded by FLJ12078, corresponding to a HOST TARGET binding site such as BINDING SITE I, BINDING SITE II or BINDING SITE III. Table 2 illustrates the complementarity of the nucleotide sequences of FLJ12078 BINDING SITE, designated SEQ ID:2121, to the nucleotide sequence of VGAM188 RNA, herein designated VGAM RNA, also designated SEQ ID:523.

Another function of VGAM188 is therefore inhibition of FLJ12078 (Accession NM_{—}024977). Accordingly, utilities of VGAM188 include diagnosis, prevention and treatment of diseases and clinical conditions associated with FLJ12078. KIAA0007 (Accession XM_{—}087089) is another VGAM188 host target gene. KIAA0007 BINDING SITE is HOST TARGET binding site found in the 3′ untranslated region of mRNA encoded by KIAA0007, corresponding to a HOST TARGET binding site such as BINDING SITE I, BINDING SITE II or BINDING SITE III. Table 2 illustrates the complementarity of the nucleotide sequences of KIAA0007 BINDING SITE, designated SEQ ID:3154, to the nucleotide sequence of VGAM188 RNA, herein designated VGAM RNA, also designated SEQ ID:523.

Another function of VGAM188 is therefore inhibition of KIAA0007 (Accession XM_{—}087089). Accordingly, utilities of VGAM188 include diagnosis, prevention and treatment of diseases and clinical conditions associated with KIAA0007. KIAA1728 (Accession XM_{—}043492) is another VGAM188 host target gene. KIAA1728 BINDING SITE is HOST TARGET binding site found in the 3′ untranslated region of mRNA encoded by KIAA1728, corresponding to a HOST TARGET binding site such as BINDING SITE I, BINDING SITE II or BINDING SITE III. Table 2 illustrates the complementarity of the nucleotide sequences of KIAA1728 BINDING SITE, designated SEQ ID:2819, to the nucleotide sequence of VGAM188 RNA, herein designated VGAM RNA, also designated SEQ ID:523.

Another function of VGAM188 is therefore inhibition of KIAA1728 (Accession XM_{—}043492). Accordingly, utilities of VGAM188 include diagnosis, prevention and treatment of diseases and clinical conditions associated with KIAA1728. MGC4643 (Accession NM_{—}032715) is another VGAM188 host target gene. MGC4643 BINDING SITE is HOST TARGET binding site found in the 3′ untranslated region of mRNA encoded by MGC4643, corresponding to a HOST TARGET binding site such as BINDING SITE I, BINDING SITE II or BINDING SITE III. Table 2 illustrates the complementarity of the nucleotide sequences of MGC4643 BINDING SITE, designated SEQ ID:2272, to the nucleotide sequence of VGAM188 RNA, herein designated VGAM RNA, also designated SEQ ID:523.

Another function of VGAM188 is therefore inhibition of MGC4643 (Accession NM_{—}032715). Accordingly, utilities of VGAM188 include diagnosis, prevention and treatment of diseases and clinical conditions associated with MGC4643. MPPE1 (Accession NM_{—}023075) is another VGAM188 host target gene. MPPE1 BINDING SITE is HOST TARGET binding site found in the 3′ untranslated region of mRNA encoded by MPPE1, corresponding to a HOST TARGET binding site such as BINDING SITE I, BINDING SITE II or BINDING SITE III. Table 2 illustrates the complementarity of the nucleotide sequences of MPPE1 BINDING SITE, designated SEQ ID:2029, to the nucleotide sequence of VGAM188 RNA, herein designated VGAM RNA, also designated SEQ ID:523.

Another function of VGAM188 is therefore inhibition of MPPE1 (Accession NM_{—}023075). Accordingly, utilities of VGAM188 include diagnosis, prevention and treatment of diseases and clinical conditions associated with MPPE1. RNA Binding Protein S1, Serine-rich Domain (RNPS1, Accession NM_{—}080594) is another VGAM188 host target gene. RNPS1 BINDING SITE1 and RNPS1 BINDING SITE2 are HOST TARGET binding sites found in untranslated regions of mRNA encoded by RNPS1, corresponding to HOST TARGET binding sites such as BINDING SITE I, BINDING SITE II or BINDING SITE III. Table 2 illustrates the complementarity of the nucleotide sequences of RNPS1 BINDING SITE1 and RNPS1 BINDING SITE2, designated SEQ ID:2376 and SEQ ID:1326 respectively, to the nucleotide sequence of VGAM188 RNA, herein designated VGAM RNA, also designated SEQ ID:523.

Another function of VGAM188 is therefore inhibition of RNA Binding Protein S1, Serine-rich Domain (RNPS1, Accession NM_{—}080594). Accordingly, utilities of VGAM188 include diagnosis, prevention and treatment of diseases and clinical conditions associated with RNPS1. LOC153027 (Accession XM_{—}041221) is another VGAM188 host target gene. LOC153027 BINDING SITE is HOST TARGET binding site found in the 3′ untranslated region of mRNA encoded by LOC153027, corresponding to a HOST TARGET binding site such as BINDING SITE I, BINDING SITE II or BINDING SITE III. Table 2 illustrates the complementarity of the nucleotide sequences of LOC153027 BINDING SITE, designated SEQ ID:2792, to the nucleotide sequence of VGAM188 RNA, herein designated VGAM RNA, also designated SEQ ID:523.

Another function of VGAM188 is therefore inhibition of LOC153027 (Accession XM_{—}041221). Accordingly, utilities of VGAM188 include diagnosis, prevention and treatment of diseases and clinical conditions associated with LOC153027. LOC153114 (Accession XM_{—}098313) is another VGAM188 host target gene. LOC153114 BINDING SITE is HOST TARGET binding site found in the 5′ untranslated region of mRNA encoded by LOC153114, corresponding to a HOST TARGET binding site such as BINDING SITE I, BINDING SITE II or BINDING SITE III. Table 2 illustrates the complementarity of the nucleotide sequences of LOC153114 BINDING SITE, designated SEQ ID:3367, to the nucleotide sequence of VGAM188 RNA, herein designated VGAM RNA, also designated SEQ ID:523.

Another function of VGAM188 is therefore inhibition of LOC153114 (Accession XM_{—}098313). Accordingly, utilities of VGAM188 include diagnosis, prevention and treatment of diseases and clinical conditions associated with LOC153114. FIG. 1 further provides a conceptual description of a novel bioinformatically detected viral gene of the present invention, referred to here as Viral Genomic Address Messenger 189 (VGAM189) viral gene, which modulates expression of respective host target genes thereof, the function and utility of which host target genes is known in the art.

VGAM189 is a novel bioinformatically detected regulatory, non protein coding, viral micro RNA (miRNA) gene. The method by which VGAM189 was detected is described hereinabove with reference to FIGS. 1-8.

VGAM189 gene, herein designated VGAM GENE, is a viral gene contained in the genome of Vaccinia Virus. VGAM189 host target gene, herein designated VGAM HOST TARGET GENE, is a human gene contained in the human genome.

VGAM189 gene encodes a VGAM189 precursor RNA, herein designated VGAM PRECURSOR RNA. Similar to other miRNA genes, and unlike most ordinary genes, VGAM189 precursor RNA does not encode a protein. A nucleotide sequence identical or highly similar to the nucleotide sequence of VGAM189 precursor RNA is designated SEQ ID:175, and is provided hereinbelow with reference to the sequence listing part. Nucleotide sequence SEQ ID:175 is located at position 96362 relative to the genome of Vaccinia Virus.

VGAM189 precursor RNA folds onto itself, forming VGAM189 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM189 folded precursor RNA into VGAM189 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM189 RNA is designated SEQ ID:524, and is provided hereinbelow with reference to the sequence listing part.

VGAM189 host target gene, herein designated VGAM HOST TARGET GENE, encodes a corresponding messenger RNA, VGAM189 host target RNA, herein designated VGAM HOST TARGET RNA. VGAM189 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM189 RNA, herein designated VGAM RNA, binds complementarily to one or more host target binding sites located in untranslated regions of VGAM189 host target RNA, herein designated VGAM HOST TARGET RNA. This complementary binding is due to the fact that the nucleotide sequence of VGAM189 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM189 RNA, herein designated VGAM RNA, may have a different number of host target binding sites in untranslated regions of a VGAM189 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM189 RNA, herein designated VGAM RNA, to host target binding sites on VGAM189 host target RNA, herein designated VGAM HOST TARGET RNA, such as BINDING SITE I, BINDING SITE II and BINDING SITE III, inhibits translation of VGAM189 host target RNA into VGAM189 host target protein, herein designated VGAM HOST TARGET PROTEIN. VGAM host target protein is therefore outlined by a broken line.

It is appreciated that VGAM189 host target gene, herein designated VGAM HOST TARGET GENE, in fact represents a plurality of VGAM189 host target genes. The mRNA of each one of this plurality of VGAM189 host target genes comprises one or more host target binding sites, each having a nucleotide sequence which is at least partly complementary to VGAM189 RNA, herein designated VGAM RNA, and which when bound by VGAM189 RNA causes inhibition of translation of respective one or more VGAM189 host target proteins.

It is further appreciated by one skilled in the art that the mode of translational inhibition illustrated by FIG. 1 with specific reference to translational inhibition exerted by VGAM189 gene, herein designated VGAM GENE, on one or more VGAM189 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM189 is inhibition of expression of host target genes, as part of a novel viral mechanism of attacking a host. Accordingly, utilities of VGAM189 include diagnosis, prevention and treatment of viral infection by Vaccinia Virus. Specific functions, and accordingly utilities, of VGAM189 correlate with, and may be deduced from, the identity of the host target genes which VGAM189 binds and inhibits, and the function of these host target genes, as elaborated hereinbelow.

Nucleotide sequences of the VGAM189 precursor RNA, herein designated VGAM PRECURSOR RNA, and of the ‘diced’ VGAM189 RNA, herein designated VGAM RNA, and a schematic representation of the secondary folding of VGAM189 folded precursor RNA, herein designated VGAM FOLDED PRECURSOR RNA, of VGAM189 are further described hereinbelow with reference to Table 1.

Nucleotide sequences of host target binding sites, such as BINDING SITE-I, BINDING SITE-II and BINDING SITE-III of FIG. 1, found on VGAM189 host target RNA, and schematic representation of the complementarity of each of these host target binding sites to VGAM189 RNA, herein designated VGAM RNA, are described hereinbelow with reference to Table 2.

As mentioned hereinabove with reference to FIG. 1, a function of VGAM189 gene, herein designated VGAM is inhibition of expression of VGAM189 target genes. It is appreciated that specific functions, and accordingly utilities, of VGAM189 correlate with, and may be deduced from, the identity of the target genes which VGAM189 binds and inhibits, and the function of these target genes, as elaborated hereinbelow.

Sortilin 1 (SORT1, Accession NM_{—}002959) is a VGAM189 host target gene. SORT1 BINDING SITE is HOST TARGET binding site found in the 3′ untranslated region of mRNA encoded by SORT1, corresponding to a HOST TARGET binding site such as BINDING SITE I, BINDING SITE II or BINDING SITE III. Table 2 illustrates the complementarity of the nucleotide sequences of SORT1 BINDING SITE, designated SEQ ID:974, to the nucleotide sequence of VGAM189 RNA, herein designated VGAM RNA, also designated SEQ ID:524.

A function of VGAM189 is therefore inhibition of Sortilin 1 (SORT1, Accession NM_{—}002959). Accordingly, utilities of VGAM189 include diagnosis, prevention and treatment of diseases and clinical conditions associated with SORT1. ATPase, Class II, Type 9A (ATP9A, Accession XM_{—}030577) is another VGAM189 host target gene. ATP9A BINDING SITE is HOST TARGET binding site found in the 3′ untranslated region of mRNA encoded by ATP9A, corresponding to a HOST TARGET binding site such as BINDING SITE I, BINDING SITE II or BINDING SITE III. Table 2 illustrates the complementarity of the nucleotide sequences of ATP9A BINDING SITE, designated SEQ ID:2618, to the nucleotide sequence of VGAM189 RNA, herein designated VGAM RNA, also designated SEQ ID:524.

Another function of VGAM189 is therefore inhibition of ATPase, Class II, Type 9A (ATP9A, Accession XM_{—}030577). Accordingly, utilities of VGAM189 include diagnosis, prevention and treatment of diseases and clinical conditions associated with ATP9A. DKFZP434K1772 (Accession XM_{—}041936) is another VGAM189 host target gene. DKFZP434K1772 BINDING SITE is HOST TARGET binding site found in the 3′ untranslated region of mRNA encoded by DKFZP434K1772, corresponding to a HOST TARGET binding site such as BINDING SITE I, BINDING SITE II or BINDING SITE III. Table 2 illustrates the complementarity of the nucleotide sequences of DKFZP434K1772 BINDING SITE, designated SEQ ID:2797, to the nucleotide sequence of VGAM189 RNA, herein designated VGAM RNA, also designated SEQ ID:524.

Another function of VGAM189 is therefore inhibition of DKFZP434K1772 (Accession XM_{—}041936). Accordingly, utilities of VGAM189 include diagnosis, prevention and treatment of diseases and clinical conditions associated with DKFZP434K1772. Unc-5 Homolog D (C. elegans) (UNC5D, Accession NM_{—}080872) is another VGAM189 host target gene. UNC5D BINDING SITE is HOST TARGET binding site found in the 3′ untranslated region of mRNA encoded by UNC5D, corresponding to a HOST TARGET binding site such as BINDING SITE I, BINDING SITE II or BINDING SITE III. Table 2 illustrates the complementarity of the nucleotide sequences of UNC5D BINDING SITE, designated SEQ ID:2392, to the nucleotide sequence of VGAM189 RNA, herein designated VGAM RNA, also designated SEQ ID:524.

Another function of VGAM189 is therefore inhibition of Unc-5 Homolog D (C. elegans) (UNC5D, Accession NM_{—}080872). Accordingly, utilities of VGAM189 include diagnosis, prevention and treatment of diseases and clinical conditions associated with UNC5D. FIG. 1 further provides a conceptual description of a novel bioinformatically detected viral gene of the present invention, referred to here as Viral Genomic Address Messenger 190 (VGAM190) viral gene, which modulates expression of respective host target genes thereof, the function and utility of which host target genes is known in the art.

VGAM190 is a novel bioinformatically detected regulatory, non protein coding, viral micro RNA (miRNA) gene. The method by which VGAM190 was detected is described hereinabove with reference to FIGS. 1-8.

VGAM190 gene, herein designated VGAM GENE, is a viral gene contained in the genome of Vaccinia Virus. VGAM190 host target gene, herein designated VGAM HOST TARGET GENE, is a human gene contained in the human genome.

VGAM190 gene encodes a VGAM190 precursor RNA, herein designated VGAM PRECURSOR RNA. Similar to other miRNA genes, and unlike most ordinary genes, VGAM190 precursor RNA does not encode a protein. A nucleotide sequence identical or highly similar to the nucleotide sequence of VGAM190 precursor RNA is designated SEQ ID:176, and is provided hereinbelow with reference to the sequence listing part. Nucleotide sequence SEQ ID:176 is located at position 100470 relative to the genome of Vaccinia Virus.

VGAM190 precursor RNA folds onto itself, forming VGAM190 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM190 folded precursor RNA into VGAM190 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM190 RNA is designated SEQ ID:525, and is provided hereinbelow with reference to the sequence listing part.

VGAM190 host target gene, herein designated VGAM HOST TARGET GENE, encodes a corresponding messenger RNA, VGAM190 host target RNA, herein designated VGAM HOST TARGET RNA. VGAM190 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM190 RNA, herein designated VGAM RNA, binds complementarily to one or more host target binding sites located in untranslated regions of VGAM190 host target RNA, herein designated VGAM HOST TARGET RNA. This complementary binding is due to the fact that the nucleotide sequence of VGAM190 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM190 RNA, herein designated VGAM RNA, may have a different number of host target binding sites in untranslated regions of a VGAM190 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM190 RNA, herein designated VGAM RNA, to host target binding sites on VGAM190 host target RNA, herein designated VGAM HOST TARGET RNA, such as BINDING SITE I, BINDING SITE II and BINDING SITE III, inhibits translation of VGAM190 host target RNA into VGAM190 host target protein, herein designated VGAM HOST TARGET PROTEIN. VGAM host target protein is therefore outlined by a broken line.

It is appreciated that VGAM190 host target gene, herein designated VGAM HOST TARGET GENE, in fact represents a plurality of VGAM190 host target genes. The mRNA of each one of this plurality of VGAM190 host target genes comprises one or more host target binding sites, each having a nucleotide sequence which is at least partly complementary to VGAM190 RNA, herein designated VGAM RNA, and which when bound by VGAM190 RNA causes inhibition of translation of respective one or more VGAM190 host target proteins.

It is further appreciated by one skilled in the art that the mode of translational inhibition illustrated by FIG. 1 with specific reference to translational inhibition exerted by VGAM190 gene, herein designated VGAM GENE, on one or more VGAM190 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM190 is inhibition of expression of host target genes, as part of a novel viral mechanism of attacking a host. Accordingly, utilities of VGAM190 include diagnosis, prevention and treatment of viral infection by Vaccinia Virus. Specific functions, and accordingly utilities, of VGAM190 correlate with, and may be deduced from, the identity of the host target genes which VGAM190 binds and inhibits, and the function of these host target genes, as elaborated hereinbelow.

Nucleotide sequences of the VGAM190 precursor RNA, herein designated VGAM PRECURSOR RNA, and of the ‘diced’ VGAM190 RNA, herein designated VGAM RNA, and a schematic representation of the secondary folding of VGAM190 folded precursor RNA, herein designated VGAM FOLDED PRECURSOR RNA, of VGAM190 are further described hereinbelow with reference to Table 1.

Nucleotide sequences of host target binding sites, such as BINDING SITE-I, BINDING SITE-II and BINDING SITE-III of FIG. 1, found on VGAM190 host target RNA, and schematic representation of the complementarity of each of these host target binding sites to VGAM190 RNA, herein designated VGAM RNA, are described hereinbelow with reference to Table 2.

As mentioned hereinabove with reference to FIG. 1, a function of VGAM190 gene, herein designated VGAM is inhibition of expression of VGAM190 target genes. It is appreciated that specific functions, and accordingly utilities, of VGAM190 correlate with, and may be deduced from, the identity of the target genes which VGAM190 binds and inhibits, and the function of these target genes, as elaborated hereinbelow.

Stearoyl-CoA Desaturase (delta-9-desaturase) (SCD, Accession NM_{—}005063) is a VGAM190 host target gene. SCD BINDING SITE is HOST TARGET binding site found in the 3′ untranslated region of mRNA encoded by SCD, corresponding to a HOST TARGET binding site such as BINDING SITE I, BINDING SITE II or BINDING SITE III. Table 2 illustrates the complementarity of the nucleotide sequences of SCD BINDING SITE, designated SEQ ID:1176, to the nucleotide sequence of VGAM190 RNA, herein designated VGAM RNA, also designated SEQ ID:525.

A function of VGAM190 is therefore inhibition of Stearoyl-CoA Desaturase (delta-9-desaturase) (SCD, Accession NM_{—}005063), a gene which functions in the synthesis of unsaturated fatty acids. Accordingly, utilities of VGAM190 include diagnosis, prevention and treatment of diseases and clinical conditions associated with SCD. The function of SCD and its association with various diseases and clinical conditions, has been established by previous studies, as described hereinabove with reference to VGAM155. Son of Sevenless Homolog 2 (Drosophila) (SOS2, Accession XM_{—}043720) is another VGAM190 host target gene. SOS2 BINDING SITE is HOST TARGET binding site found in the 3′ untranslated region of mRNA encoded by SOS2, corresponding to a HOST TARGET binding site such as BINDING SITE I, BINDING SITE II or BINDING SITE III. Table 2 illustrates the complementarity of the nucleotide sequences of SOS2 BINDING SITE, designated SEQ ID:2824, to the nucleotide sequence of VGAM190 RNA, herein designated VGAM RNA, also designated SEQ ID:525.

Another function of VGAM190 is therefore inhibition of Son of Sevenless Homolog 2 (Drosophila) (SOS2, Accession XM_{—}043720). Accordingly, utilities of VGAM190 include diagnosis, prevention and treatment of diseases and clinical conditions associated with SOS2. BANP (Accession XM_{—}038696) is another VGAM190 host target gene. BANP BINDING SITE is HOST TARGET binding site found in the 3′ untranslated region of mRNA encoded by BANP, corresponding to a HOST TARGET binding site such as BINDING SITE I, BINDING SITE II or BINDING SITE III. Table 2 illustrates the complementarity of the nucleotide sequences of BANP BINDING SITE, designated SEQ ID:2744, to the nucleotide sequence of VGAM190 RNA, herein designated VGAM RNA, also designated SEQ ID:525.

Another function of VGAM190 is therefore inhibition of BANP (Accession XM_{—}038696). Accordingly, utilities of VGAM190 include diagnosis, prevention and treatment of diseases and clinical conditions associated with BANP. KIAA0446 (Accession XM_{—}044155) is another VGAM190 host target gene. KIAA0446 BINDING SITE is HOST TARGET binding site found in the 5′ untranslated region of mRNA encoded by KIAA0446, corresponding to a HOST TARGET binding site such as BINDING SITE I, BINDING SITE II or BINDING SITE III. Table 2 illustrates the complementarity of the nucleotide sequences of KIAA0446 BINDING SITE, designated SEQ ID:2831, to the nucleotide sequence of VGAM190 RNA, herein designated VGAM RNA, also designated SEQ ID:525.

Another function of VGAM190 is therefore inhibition of KIAA0446 (Accession XM_{—}044155). Accordingly, utilities of VGAM190 include diagnosis, prevention and treatment of diseases and clinical conditions associated with KIAA0446. LOC115110 (Accession XM_{—}049825) is another VGAM190 host target gene. LOC115110 BINDING SITE is HOST TARGET binding site found in the 3′ untranslated region of mRNA encoded by LOC115110, corresponding to a HOST TARGET binding site such as BINDING SITE I, BINDING SITE II or BINDING SITE III. Table 2 illustrates the complementarity of the nucleotide sequences of LOC115110 BINDING SITE, designated SEQ ID:2924, to the nucleotide sequence of VGAM190 RNA, herein designated VGAM RNA, also designated SEQ ID:525.

Another function of VGAM190 is therefore inhibition of LOC115110 (Accession XM_{—}049825). Accordingly, utilities of VGAM190 include diagnosis, prevention and treatment of diseases and clinical conditions associated with LOC115110. LOC148443 (Accession XM_{—}086196) is another VGAM190 host target gene. LOC148443 BINDING SITE is HOST TARGET binding site found in the 3′ untranslated region of mRNA encoded by LOC148443, corresponding to a HOST TARGET binding site such as BINDING SITE I, BINDING SITE II or BINDING SITE III. Table 2 illustrates the complementarity of the nucleotide sequences of LOC148443 BINDING SITE, designated SEQ ID:3118, to the nucleotide sequence of VGAM190 RNA, herein designated VGAM RNA, also designated SEQ ID:525.

Another function of VGAM190 is therefore inhibition of LOC148443 (Accession XM_{—}086196). Accordingly, utilities of VGAM190 include diagnosis, prevention and treatment of diseases and clinical conditions associated with LOC148443. LOC151826 (Accession XM_{—}087312) is another VGAM190 host target gene. LOC151826 BINDING SITE is HOST TARGET binding site found in the 3′ untranslated region of mRNA encoded by LOC151826, corresponding to a HOST TARGET binding site such as BINDING SITE I, BINDING SITE II or BINDING SITE III. Table 2 illustrates the complementarity of the nucleotide sequences of LOC151826 BINDING SITE, designated SEQ ID:3161, to the nucleotide sequence of VGAM190 RNA, herein designated VGAM RNA, also designated SEQ ID:525.

Another function of VGAM190 is therefore inhibition of LOC151826 (Accession XM_{—}087312). Accordingly, utilities of VGAM190 include diagnosis, prevention and treatment of diseases and clinical conditions associated with LOC151826. LOC200609 (Accession XM_{—}117256) is another VGAM190 host target gene. LOC200609 BINDING SITE is HOST TARGET binding site found in the 5′ untranslated region of mRNA encoded by LOC200609, corresponding to a HOST TARGET binding site such as BINDING SITE I, BINDING SITE II or BINDING SITE III. Table 2 illustrates the complementarity of the nucleotide sequences of LOC200609 BINDING SITE, designated SEQ ID:3479, to the nucleotide sequence of VGAM190 RNA, herein designated VGAM RNA, also designated SEQ ID:525.

Another function of VGAM190 is therefore inhibition of LOC200609 (Accession XM_{—}117256). Accordingly, utilities of VGAM190 include diagnosis, prevention and treatment of diseases and clinical conditions associated with LOC200609. FIG. 1 further provides a conceptual description of a novel bioinformatically detected viral gene of the present invention, referred to here as Viral Genomic Address Messenger 191 (VGAM191) viral gene, which modulates expression of respective host target genes thereof, the function and utility of which host target genes is known in the art.

VGAM191 is a novel bioinformatically detected regulatory, non protein coding, viral micro RNA (miRNA) gene. The method by which VGAM191 was detected is described hereinabove with reference to FIGS. 1-8.

VGAM191 gene, herein designated VGAM GENE, is a viral gene contained in the genome of Vaccinia Virus. VGAM191 host target gene, herein designated VGAM HOST TARGET GENE, is a human gene contained in the human genome.

VGAM191 gene encodes a VGAM191 precursor RNA, herein designated VGAM PRECURSOR RNA. Similar to other miRNA genes, and unlike most ordinary genes, VGAM191 precursor RNA does not encode a protein. A nucleotide sequence identical or highly similar to the nucleotide sequence of VGAM191 precursor RNA is designated SEQ ID:177, and is provided hereinbelow with reference to the sequence listing part. Nucleotide sequence SEQ ID:177 is located at position 99909 relative to the genome of Vaccinia Virus.

VGAM191 precursor RNA folds onto itself, forming VGAM191 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM191 folded precursor RNA into VGAM191 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM191 RNA is designated SEQ ID:526, and is provided hereinbelow with reference to the sequence listing part.

VGAM191 host target gene, herein designated VGAM HOST TARGET GENE, encodes a corresponding messenger RNA, VGAM191 host target RNA, herein designated VGAM HOST TARGET RNA. VGAM191 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM191 RNA, herein designated VGAM RNA, binds complementarily to one or more host target binding sites located in untranslated regions of VGAM191 host target RNA, herein designated VGAM HOST TARGET RNA. This complementary binding is due to the fact that the nucleotide sequence of VGAM191 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM191 RNA, herein designated VGAM RNA, may have a different number of host target binding sites in untranslated regions of a VGAM191 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM191 RNA, herein designated VGAM RNA, to host target binding sites on VGAM191 host target RNA, herein designated VGAM HOST TARGET RNA, such as BINDING SITE I, BINDING SITE II and BINDING SITE III, inhibits translation of VGAM191 host target RNA into VGAM191 host target protein, herein designated VGAM HOST TARGET PROTEIN. VGAM host target protein is therefore outlined by a broken line.

It is appreciated that VGAM191 host target gene, herein designated VGAM HOST TARGET GENE, in fact represents a plurality of VGAM191 host target genes. The mRNA of each one of this plurality of VGAM191 host target genes comprises one or more host target binding sites, each having a nucleotide sequence which is at least partly complementary to VGAM191 RNA, herein designated VGAM RNA, and which when bound by VGAM191 RNA causes inhibition of translation of respective one or more VGAM191 host target proteins.

It is further appreciated by one skilled in the art that the mode of translational inhibition illustrated by FIG. 1 with specific reference to translational inhibition exerted by VGAM191 gene, herein designated VGAM GENE, on one or more VGAM191 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM191 is inhibition of expression of host target genes, as part of a novel viral mechanism of attacking a host. Accordingly, utilities of VGAM191 include diagnosis, prevention and treatment of viral infection by Vaccinia Virus. Specific functions, and accordingly utilities, of VGAM191 correlate with, and may be deduced from, the identity of the host target genes which VGAM191 binds and inhibits, and the function of these host target genes, as elaborated hereinbelow.

Nucleotide sequences of the VGAM191 precursor RNA, herein designated VGAM PRECURSOR RNA, and of the ‘diced’ VGAM191 RNA, herein designated VGAM RNA, and a schematic representation of the secondary folding of VGAM191 folded precursor RNA, herein designated VGAM FOLDED PRECURSOR RNA, of VGAM191 are further described hereinbelow with reference to Table 1.

Nucleotide sequences of host target binding sites, such as BINDING SITE-I, BINDING SITE-II and BINDING SITE-III of FIG. 1, found on VGAM191 host target RNA, and schematic representation of the complementarity of each of these host target binding sites to VGAM191 RNA, herein designated VGAM RNA, are described hereinbelow with reference to Table 2.

As mentioned hereinabove with reference to FIG. 1, a function of VGAM191 gene, herein designated VGAM is inhibition of expression of VGAM191 target genes. It is appreciated that specific functions, and accordingly utilities, of VGAM191 correlate with, and may be deduced from, the identity of the target genes which VGAM191 binds and inhibits, and the function of these target genes, as elaborated hereinbelow.

Basic Helix-loop-helix Domain Containing, Class B, 3 (BHLHB3, Accession NM_{—}030762) is a VGAM191 host target gene. BHLHB3 BINDING SITE is HOST TARGET binding site found in the 3′ untranslated region of mRNA encoded by BHLHB3, corresponding to a HOST TARGET binding site such as BINDING SITE I, BINDING SITE II or BINDING SITE III. Table 2 illustrates the complementarity of the nucleotide sequences of BHLHB3 BINDING SITE, designated SEQ ID:2163, to the nucleotide sequence of VGAM191 RNA, herein designated VGAM RNA, also designated SEQ ID:526.

A function of VGAM191 is therefore inhibition of Basic Helix-loop-helix Domain Containing, Class B, 3 (BHLHB3, Accession NM_{—}030762), a gene which represses both basal and activated transcription. Accordingly, utilities of VGAM191 include diagnosis, prevention and treatment of diseases and clinical conditions associated with BHLHB3. The function of BHLHB3 and its association with various diseases and clinical conditions, has been established by previous studies, as described hereinabove with reference to VGAM127. IL2-inducible T-cell Kinase (ITK, Accession NM_{—}005546) is another VGAM191 host target gene. ITK BINDING SITE is HOST TARGET binding site found in the 3′ untranslated region of mRNA encoded by ITK, corresponding to a HOST TARGET binding site such as BINDING SITE I, BINDING SITE II or BINDING SITE III. Table 2 illustrates the complementarity of the nucleotide sequences of ITK BINDING SITE, designated SEQ ID:1223, to the nucleotide sequence of VGAM191 RNA, herein designated VGAM RNA, also designated SEQ ID:526.

Another function of VGAM191 is therefore inhibition of IL2-inducible T-cell Kinase (ITK, Accession NM_{—}005546), a gene which plays a role in t cell proliferation and differentiation. Accordingly, utilities of VGAM191 include diagnosis, prevention and treatment of diseases and clinical conditions associated with ITK. The function of ITK has been established by previous studies. Signal transduction through the T-cell receptor (TCR; OMIM Ref. No. 186880) and cytokine receptors on the surface of T lymphocytes occurs largely via tyrosine phosphorylation of intracellular substrates. Signal transduction is thought to occur via association of these receptors with intracellular protein tyrosine kinases. To identify unique T-cell tyrosine kinases, Gibson et al. (1993) used PCR-based cloning with degenerate oligonucleotides directed at highly conserved motifs of tyrosine kinase domains. In this way, they cloned the complete cDNA for a unique human tyrosine kinase that is expressed mainly in T lymphocytes and natural killer (NK) cells. The cDNA predicted an open reading frame of 1,866 bp encoding a protein with a predicted size of 72 kD, which was in keeping with its size on Western blotting. A single 6.2-kb mRNA and 72-kD protein were detected in T lymphocytes and NK-like cell lines, but were not detected in other cell lineages. Sequence comparisons suggested that the protein is probably the human homolog of a murine interleukin-2-inducible T-cell kinase (ITK). However, unlike ITK, the message and protein levels for the new entity did not vary markedly on stimulation of human IL-2 responsive T cells with IL-2. They referred to the gene and its protein product as EMT (‘expressed mainly in T cells’). They concluded that EMT is a member of a new family of intracellular kinases that includes BPK (the kinase mutant in X-linked agammaglobulinemia, 300300). The expression of EMT message and protein in thymocytes and mature T cells, combined with its homology to BPK and its chromosomal localization, suggested that EMT may play a role in thymic ontogeny and growth regulation of mature T cells. Integrin adhesion receptors mediate critical interactions of T cells with other cells and extracellular matrix components during trafficking, as well as during antigen-specific recognition events in tissue. Phosphatidylinositol 3-kinase (PI3K; OMIM Ref. No. 601232) has a role in the regulation of integrin activity by CD3 (see OMIM Ref. No. 186790)-TCR and in the regulation of ITK. Woods et al. (2001) determined that TCR-mediated activation of beta-1 integrins (see OMIM Ref. No. ITGB1; 135630) requires activation of ITK and PI3K-dependent recruitment of ITK to detergent-insoluble glycosphingolipid-enriched microdomains (DIGs) via binding of the pleckstrin homology domain of ITK to the PI3K product PI(3,4,5)-P3. Likewise, activation of PI3K and LCK (OMIM Ref. No. 153390) via CD4 (OMIM Ref. No. 186940) coreceptor stimulation can initiate beta-1 integrin activation dependent on ITK function. CD4 stimulation, together with targeting of ITK to DIGs, also activates TCR-independent beta-1 integrin function. Changes in beta-1 integrin function mediated by TCR-induced activation of ITK are accompanied by ITK-dependent modulation of the actin cytoskeleton. Woods et al. (2001) concluded that TCR-mediated activation of beta-1 integrin involves membrane relocalization and activation of ITK via coordinate action of PI3K and an SRC family tyrosine kinase. Animal model experiments lend further support to the function of ITK. By homologous recombination, Schaeffer et al. (1999) disrupted the Rlk (TXK; 600058) gene in mice. Heterozygotes were completely normal. Homozygous null Rlk mice showed increased amounts of Itk mRNA. The authors hypothesized that up-regulation of related Tec kinases may partially compensate for the lack of Rlk. Schaeffer et al. (1999) therefore generated Rlk −/− Itk −/− mice by interbreeding. Itk-deficient mice have reduced numbers of mature T cells, particularly CD4+ cells, causing a decreased CD4-to-CD8 ratio. Rlk −/− Itk −/− mutants, however, had normal T cell numbers. Both CD4+ and CD8+ cell numbers are increased relative to Itk −/− mice. The persistent abnormal ratio of CD4+ to CD8+ cells suggested an altered regulation of lymphoid development and homeostasis in the double mutants. The double mutants had marked defects in T-cell receptor responses including proliferation, cytokine production, and apoptosis in vitro and adaptive immune responses to Toxoplasma gondii in vivo. Molecular events immediately downstream from the T-cell receptor were intact in Rlk −/− Itk −/− cells, but intermediate events including inositol trisphosphate production, calcium mobilization, and mitogen-activated protein kinase activation were impaired, establishing Tec kinases as critical regulators of T-cell receptor signaling required for phospholipase C-gamma activation.

It is appreciated that the abovementioned animal model for ITK is acknowledged by those skilled in the art as a scientifically valid animal model, as can be further appreciated from the publications sited hereinbelow.

Full details of the abovementioned studies are described in the following publications, the disclosure of which are hereby incorporated by reference:
Schaeffer, E. M.; Debnath, J.; Yap, G.; McVicar, D.; Liao, X. C.; Littman, D. R.; Sher, A.; Varmus, H. E.; Lenardo, M. J.; Schwartzberg, P. L.: Requirement for Tec kinases Rlk and Itk in T cell receptor signaling and immunity. Science 284: 638-641, 1999.; and
Woods, M. L.; Kivens, W. J.; Adelsman, M. A.; Qiu, Y.; August, A.; Shimizu, Y.: A novel function for the Tec family tyrosine kinase Itk in activation of beta-1 integrins by the T-cell.

Further studies establishing the function and utilities of ITK are found in John Hopkins OMIM database record ID 186973, and in sited publications numbered 266-270 listed in the bibliography section hereinbelow, which are also hereby incorporated by reference. MpV17 Transgene, Murine Homolog, Glomerulosclerosis (MPV17, Accession XM_{—}047175) is another VGAM191 host target gene. MPV17 BINDING SITE is HOST TARGET binding site found in the 3′ untranslated region of mRNA encoded by MPV17, corresponding to a HOST TARGET binding site such as BINDING SITE I, BINDING SITE II or BINDING SITE III. Table 2 illustrates the complementarity of the nucleotide sequences of MPV17 BINDING SITE, designated SEQ ID:2888, to the nucleotide sequence of VGAM191 RNA, herein designated VGAM RNA, also designated SEQ ID:526.

Another function of VGAM191 is therefore inhibition of MpV17 Transgene, Murine Homolog, Glomerulosclerosis (MPV17, Accession XM_{—}047175), a gene which seems implicated in the metabolism of reactive oxygen species (by similarity). Accordingly, utilities of VGAM191 include diagnosis, prevention and treatment of diseases and clinical conditions associated with MPV17. The function of MPV17 has been established by previous studies. The Mpv17 strain of mice carries a transgenically produced retroviral insert in its genome (Weiher et al., 1990). The integration prevents the expression of the MPV17 gene, leading to the development of glomerulosclerosis in mice homozygous for the integration. Physiologically and histologically, the phenotype of the kidney disease resembles human glomerulosclerosis. The mice demonstrate nephrotic syndrome at an early age. Karasawa et al. (1993) isolated cDNA clones representing a single RNA species for the human homolog of the Mpv17 gene. Sequence analysis demonstrated over 90% identity in the region coding for a protein of 176 amino acids and unknown function. Cloning of the genomic locus revealed a single-copy gene, which, by somatic cell hybrid analysis and in situ hybridization, was mapped to 2p23-p21. Southern analysis of rodent/mouse somatic cell hybrids demonstrated that the gene is located on mouse chromosome 5, thus defining a new region of homology between human 2p and mouse 5. Karasawa et al. (1993) demonstrated that the MPV17 coding region is not mutated in patients suffering from the Finnish type of congenital nephrotic syndrome (OMIM Ref. No. 256300).

Full details of the abovementioned studies are described in the following publications, the disclosure of which are hereby incorporated by reference:
Karasawa, M.; Zwacka, R. M.; Reuter, A.; Fink, T.; Hsieh, C. L.; Lichter, P.; Francke, U.; Weiher, H.: The human homolog of the glomerulosclerosis gene Mpv17: structure and genomic organization. Hum. Molec. Genet. 2: 1829-1834, 1993.; and
Weiher, H.; Noda, T.; Gray, D. A.; Sharpe, A. H.; Jaenisch, R.: Transgenic mouse model of kidney disease: insertional inactivation of ubiquitously expressed gene leads to nephrotic syn.

Further studies establishing the function and utilities of MPV17 are found in John Hopkins OMIM database record ID 137960, and in sited publications numbered 499 listed in the bibliography section hereinbelow, which are also hereby incorporated by reference. Macrophage Scavenger Receptor 1 (MSR1, Accession NM_{—}002445) is another VGAM191 host target gene. MSR1 BINDING SITE is HOST TARGET binding site found in the 3′ untranslated region of mRNA encoded by MSR1, corresponding to a HOST TARGET binding site such as BINDING SITE I, BINDING SITE II or BINDING SITE III. Table 2 illustrates the complementarity of the nucleotide sequences of MSR1 BINDING SITE, designated SEQ ID:930, to the nucleotide sequence of VGAM191 RNA, herein designated VGAM RNA, also designated SEQ ID:526.

Another function of VGAM191 is therefore inhibition of Macrophage Scavenger Receptor 1 (MSR1, Accession NM_{—}002445), a gene which plays a role in endocytosis of macromolecules. Accordingly, utilities of VGAM191 include diagnosis, prevention and treatment of diseases and clinical conditions associated with MSR1. The function of MSR1 and its association with various diseases and clinical conditions, has been established by previous studies, as described hereinabove with reference to VGAM49. RNA (guanine-7-) Methyltransferase (RNMT, Accession NM_{—}003799) is another VGAM191 host target gene. RNMT BINDING SITE is HOST TARGET binding site found in the 3′ untranslated region of mRNA encoded by RNMT, corresponding to a HOST TARGET binding site such as BINDING SITE I, BINDING SITE II or BINDING SITE III. Table 2 illustrates the complementarity of the nucleotide sequences of RNMT BINDING SITE, designated SEQ ID: 1061, to the nucleotide sequence of VGAM191 RNA, herein designated VGAM RNA, also designated SEQ ID:526.

Another function of VGAM191 is therefore inhibition of RNA (guanine-7-) Methyltransferase (RNMT, Accession NM_{—}003799), a gene which catalyzes the methylation of GpppN- at the guanine N7 position. Accordingly, utilities of VGAM191 include diagnosis, prevention and treatment of diseases and clinical conditions associated with RNMT. The function of RNMT and its association with various diseases and clinical conditions, has been established by previous studies, as described hereinabove with reference to VGAM55. Sterol Carrier Protein 2 (SCP2, Accession NM_{—}002979) is another VGAM191 host target gene. SCP2 BINDING SITE is HOST TARGET binding site found in the 3′ untranslated region of mRNA encoded by SCP2, corresponding to a HOST TARGET binding site such as BINDING SITE I, BINDING SITE II or BINDING SITE III. Table 2 illustrates the complementarity of the nucleotide sequences of SCP2 BINDING SITE, designated SEQ ID:976, to the nucleotide sequence of VGAM191 RNA, herein designated VGAM RNA, also designated SEQ ID:526.

Another function of VGAM191 is therefore inhibition of Sterol Carrier Protein 2 (SCP2, Accession NM_{—}002979), a gene which may regulate steroidogenesis. Accordingly, utilities of VGAM191 include diagnosis, prevention and treatment of diseases and clinical conditions associated with SCP2. The function of SCP2 has been established by previous studies. During meiotic prophase, chromosomes are arranged along proteinaceous axes called axial elements. In rat, the major protein components of axial elements are proteins of 30, 33, and 190 kD. The 30- and 33-kD proteins are closely related and appear to be products of a single gene, Scp3 (synaptonemal complex protein-3; 604759). Offenberg et al. (1998) isolated rat testis cDNAs encoding the 190-kD protein, which they designated Scp2. Sequence analysis revealed that Scp2 is a basic protein, with a pI of 8. Scp2 contains 2 clusters of S/T-P motifs, which are common in DNA-binding proteins, and a C-terminal coiled-coil region. In Southwestern blot experiments, recombinant Scp2 bound DNA. Using immunocytology, Offenberg et al. (1998) determined that Scp2 localizes specifically to the synaptonemal complex in the nuclei of rat testis meiotic prophase nuclei. Northern blot analysis indicated that Scp2 is expressed exclusively in testis. The authors noted that Scp2 shows some similarity at the amino acid sequence and secondary structural level to the S. cerevisiae Red1 protein, which is involved in meiotic recombination and the assembly of axial elements of synaptonemal complexes. They speculated that Scp2 is a DNA-binding protein involved in the structural organization of meiotic prophase chromosomes. By screening a human testis library with a partial rat Scp2 cDNA, Schalk et al. (1999) isolated cDNAs encoding human SCP2. The predicted 1,530-amino acid human protein shares 63% amino acid identity with rat Scp2. Like rat Scp2, human SCP2 contains S/T-P motifs, 2 nuclear targeting signals, and a C-terminal coiled-coil region.

Full details of the abovementioned studies are described in the following publications, the disclosure of which are hereby incorporated by reference:
Offenberg, H. H.; Schalk, J. A. C.; Meuwissen, R. L. J.; van Aalderen, M.; Kester, H. A.; Dietrich, A. J. J.; Heyting, C.: SCP2: a major protein component of the axial elements of synaptonemal complexes of the rat. Nucleic Acids Res. 26: 2572-2579, 1998.; and
Schalk, J. A. C.; Offenberg, H. H.; Peters, E.; Groot, N. P. B.; Hoovers, J. M. N.; Heyting, C.: Isolation and characterization of the human SCP2 cDNA and chromosomal localization of t.

Further studies establishing the function and utilities of SCP2 are found in John Hopkins OMIM database record ID 604105, and in sited publications numbered 1618-1619 listed in the bibliography section hereinbelow, which are also hereby incorporated by reference. DKFZP564K0822 (Accession XM_{—}168378) is another VGAM191 host target gene. DKFZP564K0822 BINDING SITE is HOST TARGET binding site found in the 3′ untranslated region of mRNA encoded by DKFZP564K0822, corresponding to a HOST TARGET binding site such as BINDING SITE I, BINDING SITE II or BINDING SITE III. Table 2 illustrates the complementarity of the nucleotide sequences of DKFZP564K0822 BINDING SITE, designated SEQ ID:3630, to the nucleotide sequence of VGAM191 RNA, herein designated VGAM RNA, also designated SEQ ID:526.

Another function of VGAM191 is therefore inhibition of DKFZP564K0822 (Accession XM_{—}168378). Accordingly, utilities of VGAM191 include diagnosis, prevention and treatment of diseases and clinical conditions associated with DKFZP564K0822. KIAA0564 (Accession XM_{—}038664) is another VGAM191 host target gene. KIAA0564 BINDING SITE is HOST TARGET binding site found in the 3′ untranslated region of mRNA encoded by KIAA0564, corresponding to a HOST TARGET binding site such as BINDING SITE I, BINDING SITE II or BINDING SITE III. Table 2 illustrates the complementarity of the nucleotide sequences of KIAA0564 BINDING SITE, designated SEQ ID:2742, to the nucleotide sequence of VGAM191 RNA, herein designated VGAM RNA, also designated SEQ ID:526.

Another function of VGAM191 is therefore inhibition of KIAA0564 (Accession XM_{—}038664). Accordingly, utilities of VGAM191 include diagnosis, prevention and treatment of diseases and clinical conditions associated with KIAA0564. KIAA0769 (Accession NM_{—}014824) is another VGAM191 host target gene. KIAA0769 BINDING SITE is HOST TARGET binding site found in the 3′ untranslated region of mRNA encoded by KIAA0769, corresponding to a HOST TARGET binding site such as BINDING SITE I, BINDING SITE II or BINDING SITE III. Table 2 illustrates the complementarity of the nucleotide sequences of KIAA0769 BINDING SITE, designated SEQ ID:1560, to the nucleotide sequence of VGAM191 RNA, herein designated VGAM RNA, also designated SEQ ID:526.

Another function of VGAM191 is therefore inhibition of KIAA0769 (Accession NM_{—}014824). Accordingly, utilities of VGAM191 include diagnosis, prevention and treatment of diseases and clinical conditions associated with KIAA0769. KIAA1163 (Accession XM_{—}086231) is another VGAM191 host target gene. KIAA1163 BINDING SITE is HOST TARGET binding site found in the 3′ untranslated region of mRNA encoded by KIAA1163, corresponding to a HOST TARGET binding site such as BINDING SITE I, BINDING SITE II or BINDING SITE III. Table 2 illustrates the complementarity of the nucleotide sequences of KIAA1163 BINDING SITE, designated SEQ ID:3122, to the nucleotide sequence of VGAM191 RNA, herein designated VGAM RNA, also designated SEQ ID:526.

Another function of VGAM191 is therefore inhibition of KIAA1163 (Accession XM_{—}086231). Accordingly, utilities of VGAM191 include diagnosis, prevention and treatment of diseases and clinical conditions associated with KIAA1163. KIAA1332 (Accession XM_{—}048774) is another VGAM191 host target gene. KIAA1332 BINDING SITE is HOST TARGET binding site found in the 3′ untranslated region of mRNA encoded by KIAA1332, corresponding to a HOST TARGET binding site such as BINDING SITE I, BINDING SITE II or BINDING SITE III. Table 2 illustrates the complementarity of the nucleotide sequences of KIAA1332 BINDING SITE, designated SEQ ID:2909, to the nucleotide sequence of VGAM191 RNA, herein designated VGAM RNA, also designated SEQ ID:526.

Another function of VGAM191 is therefore inhibition of KIAA1332 (Accession XM_{—}048774). Accordingly, utilities of VGAM191 include diagnosis, prevention and treatment of diseases and clinical conditions associated with KIAA1332. Splicing Factor, Arginine/serine-rich 11 (SFRS11, Accession NM_{—}004768) is another VGAM191 host target gene. SFRS11 BINDING SITE is HOST TARGET binding site found in the 3′ untranslated region of mRNA encoded by SFRS11, corresponding to a HOST TARGET binding site such as BINDING SITE I, BINDING SITE II or BINDING SITE III. Table 2 illustrates the complementarity of the nucleotide sequences of SFRS11 BINDING SITE, designated SEQ ID:1155, to the nucleotide sequence of VGAM191 RNA, herein designated VGAM RNA, also designated SEQ ID:526.

Another function of VGAM191 is therefore inhibition of Splicing Factor, Arginine/serine-rich 11 (SFRS11, Accession NM_{—}004768). Accordingly, utilities of VGAM191 include diagnosis, prevention and treatment of diseases and clinical conditions associated with SFRS11. Zinc Finger Protein 387 (ZNF387, Accession NM_{—}014682) is another VGAM191 host target gene. ZNF387 BINDING SITE is HOST TARGET binding site found in the 3′ untranslated region of mRNA encoded by ZNF387, corresponding to a HOST TARGET binding site such as BINDING SITE I, BINDING SITE II or BINDING SITE III. Table 2 illustrates the complementarity of the nucleotide sequences of ZNF387 BINDING SITE, designated SEQ ID:1521, to the nucleotide sequence of VGAM191 RNA, herein designated VGAM RNA, also designated SEQ ID:526.

Another function of VGAM191 is therefore inhibition of Zinc Finger Protein 387 (ZNF387, Accession NM_{—}014682). Accordingly, utilities of VGAM191 include diagnosis, prevention and treatment of diseases and clinical conditions associated with ZNF387. LOC138639 (Accession XM_{—}059988) is another VGAM191 host target gene. LOC138639 BINDING SITE is HOST TARGET binding site found in the 3′ untranslated region of mRNA encoded by LOC138639, corresponding to a HOST TARGET binding site such as BINDING SITE I, BINDING SITE II or BINDING SITE III. Table 2 illustrates the complementarity of the nucleotide sequences of LOC138639 BINDING SITE, designated SEQ ID:3020, to the nucleotide sequence of VGAM191 RNA, herein designated VGAM RNA, also designated SEQ ID:526.

Another function of VGAM191 is therefore inhibition of LOC138639 (Accession XM_{—}059988). Accordingly, utilities of VGAM191 include diagnosis, prevention and treatment of diseases and clinical conditions associated with LOC138639. LOC147299 (Accession XM_{—}085763) is another VGAM191 host target gene. LOC147299 BINDING SITE is HOST TARGET binding site found in the 3′ untranslated region of mRNA encoded by LOC147299, corresponding to a HOST TARGET binding site such as BINDING SITE I, BINDING SITE II or BINDING SITE III. Table 2 illustrates the complementarity of the nucleotide sequences of LOC147299 BINDING SITE, designated SEQ ID:3107, to the nucleotide sequence of VGAM191 RNA, herein designated VGAM RNA, also designated SEQ ID:526.

Another function of VGAM191 is therefore inhibition of LOC147299 (Accession XM_{—}085763). Accordingly, utilities of VGAM191 include diagnosis, prevention and treatment of diseases and clinical conditions associated with LOC147299. LOC90019 (Accession NM_{—}138567) is another VGAM191 host target gene. LOC90019 BINDING SITE is HOST TARGET binding site found in the 5′ untranslated region of mRNA encoded by LOC90019, corresponding to a HOST TARGET binding site such as BINDING SITE I, BINDING SITE II or BINDING SITE III. Table 2 illustrates the complementarity of the nucleotide sequences of LOC90019 BINDING SITE, designated SEQ ID:2445, to the nucleotide sequence of VGAM191 RNA, herein designated VGAM RNA, also designated SEQ ID:526.

Another function of VGAM191 is therefore inhibition of LOC90019 (Accession NM_{—}138567). Accordingly, utilities of VGAM191 include diagnosis, prevention and treatment of diseases and clinical conditions associated with LOC90019. FIG. 1 further provides a conceptual description of a novel bioinformatically detected viral gene of the present invention, referred to here as Viral Genomic Address Messenger 192 (VGAM192) viral gene, which modulates expression of respective host target genes thereof, the function and utility of which host target genes is known in the art.

VGAM192 is a novel bioinformatically detected regulatory, non protein coding, viral micro RNA (miRNA) gene. The method by which VGAM192 was detected is described hereinabove with reference to FIGS. 1-8.

VGAM192 gene, herein designated VGAM GENE, is a viral gene contained in the genome of Vaccinia Virus. VGAM192 host target gene, herein designated VGAM HOST TARGET GENE, is a human gene contained in the human genome.

VGAM192 gene encodes a VGAM192 precursor RNA, herein designated VGAM PRECURSOR RNA. Similar to other miRNA genes, and unlike most ordinary genes, VGAM192 precursor RNA does not encode a protein. A nucleotide sequence identical or highly similar to the nucleotide sequence of VGAM192 precursor RNA is designated SEQ ID:178, and is provided hereinbelow with reference to the sequence listing part. Nucleotide sequence SEQ ID:178 is located at position 102578 relative to the genome of Vaccinia Virus.

VGAM192 precursor RNA folds onto itself, forming VGAM192 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM192 folded precursor RNA into VGAM192 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM192 RNA is designated SEQ ID:527, and is provided hereinbelow with reference to the sequence listing part.

VGAM192 host target gene, herein designated VGAM HOST TARGET GENE, encodes a corresponding messenger RNA, VGAM192 host target RNA, herein designated VGAM HOST TARGET RNA. VGAM192 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM192 RNA, herein designated VGAM RNA, binds complementarily to one or more host target binding sites located in untranslated regions of VGAM192 host target RNA, herein designated VGAM HOST TARGET RNA. This complementary binding is due to the fact that the nucleotide sequence of VGAM192 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM192 RNA, herein designated VGAM RNA, may have a different number of host target binding sites in untranslated regions of a VGAM192 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM192 RNA, herein designated VGAM RNA, to host target binding sites on VGAM192 host target RNA, herein designated VGAM HOST TARGET RNA, such as BINDING SITE I, BINDING SITE II and BINDING SITE III, inhibits translation of VGAM192 host target RNA into VGAM192 host target protein, herein designated VGAM HOST TARGET PROTEIN. VGAM host target protein is therefore outlined by a broken line.

It is appreciated that VGAM192 host target gene, herein designated VGAM HOST TARGET GENE, in fact represents a plurality of VGAM192 host target genes. The mRNA of each one of this plurality of VGAM192 host target genes comprises one or more host target binding sites, each having a nucleotide sequence which is at least partly complementary to VGAM192 RNA, herein designated VGAM RNA, and which when bound by VGAM192 RNA causes inhibition of translation of respective one or more VGAM192 host target proteins.

It is further appreciated by one skilled in the art that the mode of translational inhibition illustrated by FIG. 1 with specific reference to translational inhibition exerted by VGAM192 gene, herein designated VGAM GENE, on one or more VGAM192 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM192 is inhibition of expression of host target genes, as part of a novel viral mechanism of attacking a host. Accordingly, utilities of VGAM192 include diagnosis, prevention and treatment of viral infection by Vaccinia Virus. Specific functions, and accordingly utilities, of VGAM192 correlate with, and may be deduced from, the identity of the host target genes which VGAM192 binds and inhibits, and the function of these host target genes, as elaborated hereinbelow.

Nucleotide sequences of the VGAM192 precursor RNA, herein designated VGAM PRECURSOR RNA, and of the ‘diced’ VGAM192 RNA, herein designated VGAM RNA, and a schematic representation of the secondary folding of VGAM192 folded precursor RNA, herein designated VGAM FOLDED PRECURSOR RNA, of VGAM192 are further described hereinbelow with reference to Table 1.

Nucleotide sequences of host target binding sites, such as BINDING SITE-I, BINDING SITE-II and BINDING SITE-III of FIG. 1, found on VGAM192 host target RNA, and schematic representation of the complementarity of each of these host target binding sites to VGAM192 RNA, herein designated VGAM RNA, are described hereinbelow with reference to Table 2.

As mentioned hereinabove with reference to FIG. 1, a function of VGAM192 gene, herein designated VGAM is inhibition of expression of VGAM192 target genes. It is appreciated that specific functions, and accordingly utilities, of VGAM192 correlate with, and may be deduced from, the identity of the target genes which VGAM192 binds and inhibits, and the function of these target genes, as elaborated hereinbelow.

UDP-Gal: betaGlcNAc Beta 1,3-galactosyltransferase, Polypeptide 5 (B3GALT5, Accession NM_{—}033173) is a VGAM192 host target gene. B3GALT5 BINDING SITE is HOST TARGET binding site found in the 5′ untranslated region of mRNA encoded by B3GALT5, corresponding to a HOST TARGET binding site such as BINDING SITE I, BINDING SITE II or BINDING SITE III. Table 2 illustrates the complementarity of the nucleotide sequences of B3GALT5 BINDING SITE, designated SEQ ID:2320, to the nucleotide sequence of VGAM192 RNA, herein designated VGAM RNA, also designated SEQ ID:527.

A function of VGAM192 is therefore inhibition of UDP-Gal:betaGlcNAc Beta 1,3-galactosyltransferase, Polypeptide 5 (B3GALT5, Accession NM_{—}033173). Accordingly, utilities of VGAM192 include diagnosis, prevention and treatment of diseases and clinical conditions associated with B3GALT5. Basic Transcription Element Binding Protein 1 (BTEB1, Accession NM_{—}001206) is another VGAM192 host target gene. BTEB1 BINDING SITE is HOST TARGET binding site found in the 3′ untranslated region of mRNA encoded by BTEB1, corresponding to a HOST TARGET binding site such as BINDING SITE I, BINDING SITE II or BINDING SITE III. Table 2 illustrates the complementarity of the nucleotide sequences of BTEB1 BINDING SITE, designated SEQ ID:808, to the nucleotide sequence of VGAM192 RNA, herein designated VGAM RNA, also designated SEQ ID:527.

Another function of VGAM192 is therefore inhibition of Basic Transcription Element Binding Protein 1 (BTEB1, Accession NM_{—}001206). Accordingly, utilities of VGAM192 include diagnosis, prevention and treatment of diseases and clinical conditions associated with BTEB1. Retinitis Pigmentosa 2 (X-linked recessive) (RP2, Accession NM_{—}006915) is another VGAM192 host target gene. RP2 BINDING SITE is HOST TARGET binding site found in the 3′ untranslated region of mRNA encoded by RP2, corresponding to a HOST TARGET binding site such as BINDING SITE I, BINDING SITE II or BINDING SITE III. Table 2 illustrates the complementarity of the nucleotide sequences of RP2 BINDING SITE, designated SEQ ID:1340, to the nucleotide sequence of VGAM192 RNA, herein designated VGAM RNA, also designated SEQ ID:527.

Another function of VGAM192 is therefore inhibition of Retinitis Pigmentosa 2 (X-linked recessive) (RP2, Accession NM_{—}006915). Accordingly, utilities of VGAM192 include diagnosis, prevention and treatment of diseases and clinical conditions associated with RP2. Chromosome 12 Open Reading Frame 22 (C12orf22, Accession NM_{—}030809) is another VGAM192 host target gene. C12orf22 BINDING SITE is HOST TARGET binding site found in the 3′ untranslated region of mRNA encoded by C12orf22, corresponding to a HOST TARGET binding site such as BINDING SITE I, BINDING SITE II or BINDING SITE III. Table 2 illustrates the complementarity of the nucleotide sequences of C12orf22 BINDING SITE, designated SEQ ID:2169, to the nucleotide sequence of VGAM192 RNA, herein designated VGAM RNA, also designated SEQ ID:527.

Another function of VGAM192 is therefore inhibition of Chromosome 12 Open Reading Frame 22 (C12orf22, Accession NM_{—}030809). Accordingly, utilities of VGAM192 include diagnosis, prevention and treatment of diseases and clinical conditions associated with C12orf22. Chromosome 20 Open Reading Frame 26 (C20orf26, Accession XM_{—}046598) is another VGAM192 host target gene. C20orf26 BINDING SITE is HOST TARGET binding site found in the 3′ untranslated region of mRNA encoded by C20orf26, corresponding to a HOST TARGET binding site such as BINDING SITE I, BINDING SITE II or BINDING SITE III. Table 2 illustrates the complementarity of the nucleotide sequences of C20orf26 BINDING SITE, designated SEQ ID:2879, to the nucleotide sequence of VGAM192 RNA, herein designated VGAM RNA, also designated SEQ ID:527.

Another function of VGAM192 is therefore inhibition of Chromosome 20 Open Reading Frame 26 (C20orf26, Accession XM_{—}046598). Accordingly, utilities of VGAM192 include diagnosis, prevention and treatment of diseases and clinical conditions associated with C20orf26. FLJ14627 (Accession NM_{—}032814) is another VGAM192 host target gene. FLJ14627 BINDING SITE is HOST TARGET binding site found in the 5′ untranslated region of mRNA encoded by FLJ14627, corresponding to a HOST TARGET binding site such as BINDING SITE I, BINDING SITE II or BINDING SITE III. Table 2 illustrates the complementarity of the nucleotide sequences of FLJ14627 BINDING SITE, designated SEQ ID:2283, to the nucleotide sequence of VGAM192 RNA, herein designated VGAM RNA, also designated SEQ ID:527.

Another function of VGAM192 is therefore inhibition of FLJ14627 (Accession NM_{—}032814). Accordingly, utilities of VGAM192 include diagnosis, prevention and treatment of diseases and clinical conditions associated with FLJ14627. G Protein-coupled Receptor 72 (GPR72, Accession XM_{—}045812) is another VGAM192 host target gene. GPR72 BINDING SITE is HOST TARGET binding site found in the 3′ untranslated region of mRNA encoded by GPR72, corresponding to a HOST TARGET binding site such as BINDING SITE I, BINDING SITE II or BINDING SITE III. Table 2 illustrates the complementarity of the nucleotide sequences of GPR72 BINDING SITE, designated SEQ ID:2867, to the nucleotide sequence of VGAM192 RNA, herein designated VGAM RNA, also designated SEQ ID:527.

Another function of VGAM192 is therefore inhibition of G Protein-coupled Receptor 72 (GPR72, Accession XM_{—}045812). Accordingly, utilities of VGAM192 include diagnosis, prevention and treatment of diseases and clinical conditions associated with GPR72. Glutamic Pyruvate Transaminase (alanine aminotransferase) 2 (GPT2, Accession NM_{—}133443) is another VGAM192 host target gene. GPT2 BINDING SITE is HOST TARGET binding site found in the 3′ untranslated region of mRNA encoded by GPT2, corresponding to a HOST TARGET binding site such as BINDING SITE I, BINDING SITE II or BINDING SITE III. Table 2 illustrates the complementarity of the nucleotide sequences of GPT2 BINDING SITE, designated SEQ ID:2418, to the nucleotide sequence of VGAM192 RNA, herein designated VGAM RNA, also designated SEQ ID:527.

Another function of VGAM192 is therefore inhibition of Glutamic Pyruvate Transaminase (alanine aminotransferase) 2 (GPT2, Accession NM_{—}133443). Accordingly, utilities of VGAM192 include diagnosis, prevention and treatment of diseases and clinical conditions associated with GPT2. KIAA0470 (Accession NM_{—}014812) is another VGAM192 host target gene. KIAA0470 BINDING SITE is HOST TARGET binding site found in the 5′ untranslated region of mRNA encoded by KIAA0470, corresponding to a HOST TARGET binding site such as BINDING SITE I, BINDING SITE II or BINDING SITE III. Table 2 illustrates the complementarity of the nucleotide sequences of KIAA0470 BINDING SITE, designated SEQ ID:1556, to the nucleotide sequence of VGAM192 RNA, herein designated VGAM RNA, also designated SEQ ID:527.

Another function of VGAM192 is therefore inhibition of KIAA0470 (Accession NM_{—}014812). Accordingly, utilities of VGAM192 include diagnosis, prevention and treatment of diseases and clinical conditions associated with KIAA0470. KIAA1328 (Accession XM_{—}029429) is another VGAM192 host target gene. KIAA1328 BINDING SITE is HOST TARGET binding site found in the 5′ untranslated region of mRNA encoded by KIAA1328, corresponding to a HOST TARGET binding site such as BINDING SITE I, BINDING SITE II or BINDING SITE III. Table 2 illustrates the complementarity of the nucleotide sequences of KIAA1328 BINDING SITE, designated SEQ ID:2602, to the nucleotide sequence of VGAM192 RNA, herein designated VGAM RNA, also designated SEQ ID:527.

Another function of VGAM192 is therefore inhibition of KIAA1328 (Accession XM_{—}029429). Accordingly, utilities of VGAM192 include diagnosis, prevention and treatment of diseases and clinical conditions associated with KIAA1328. KIAA1981 (Accession XM_{—}114000) is another VGAM192 host target gene. KIAA1981 BINDING SITE is HOST TARGET binding site found in the 3′ untranslated region of mRNA encoded by KIAA1981, corresponding to a HOST TARGET binding site such as BINDING SITE I, BINDING SITE II or BINDING SITE III. Table 2 illustrates the complementarity of the nucleotide sequences of KIAA1981 BINDING SITE, designated SEQ ID:3430, to the nucleotide sequence of VGAM192 RNA, herein designated VGAM RNA, also designated SEQ ID:527.

Another function of VGAM192 is therefore inhibition of KIAA1981 (Accession XM_{—}114000). Accordingly, utilities of VGAM192 include diagnosis, prevention and treatment of diseases and clinical conditions associated with KIAA1981. MGC17330 (Accession NM_{—}052880) is another VGAM192 host target gene. MGC17330 BINDING SITE is HOST TARGET binding site found in the 3′ untranslated region of mRNA encoded by MGC17330, corresponding to a HOST TARGET binding site such as BINDING SITE I, BINDING SITE II or BINDING SITE III. Table 2 illustrates the complementarity of the nucleotide sequences of MGC17330 BINDING SITE, designated SEQ ID:2342, to the nucleotide sequence of VGAM192 RNA, herein designated VGAM RNA, also designated SEQ ID:527.

Another function of VGAM192 is therefore inhibition of MGC17330 (Accession NM_{—}052880). Accordingly, utilities of VGAM192 include diagnosis, prevention and treatment of diseases and clinical conditions associated with MGC17330. Mitochondrial Ribosomal Protein L35 (MRPL35, Accession NM_{—}016622) is another VGAM192 host target gene. MRPL35 BINDING SITE is HOST TARGET binding site found in the 3′ untranslated region of mRNA encoded by MRPL35, corresponding to a HOST TARGET binding site such as BINDING SITE I, BINDING SITE II or BINDING SITE III. Table 2 illustrates the complementarity of the nucleotide sequences of MRPL35 BINDING SITE, designated SEQ ID:1701, to the nucleotide sequence of VGAM192 RNA, herein designated VGAM RNA, also designated SEQ ID:527.

Another function of VGAM192 is therefore inhibition of Mitochondrial Ribosomal Protein L35 (MRPL35, Accession NM_{—}016622). Accordingly, utilities of VGAM192 include diagnosis, prevention and treatment of diseases and clinical conditions associated with MRPL35. LOC205011 (Accession XM_{—}117741) is another VGAM192 host target gene. LOC205011 BINDING SITE is HOST TARGET binding site found in the 5′ untranslated region of mRNA encoded by LOC205011, corresponding to a HOST TARGET binding site such as BINDING SITE I, BINDING SITE II or BINDING SITE III. Table 2 illustrates the complementarity of the nucleotide sequences of LOC205011 BINDING SITE, designated SEQ ID:3492, to the nucleotide sequence of VGAM192 RNA, herein designated VGAM RNA, also designated SEQ ID:527.

Another function of VGAM192 is therefore inhibition of LOC205011 (Accession XM_{—}117741). Accordingly, utilities of VGAM192 include diagnosis, prevention and treatment of diseases and clinical conditions associated with LOC205011. FIG. 1 further provides a conceptual description of a novel bioinformatically detected viral gene of the present invention, referred to here as Viral Genomic Address Messenger 193 (VGAM193) viral gene, which modulates expression of respective host target genes thereof, the function and utility of which host target genes is known in the art.

VGAM193 is a novel bioinformatically detected regulatory, non protein coding, viral micro RNA (miRNA) gene. The method by which VGAM193 was detected is described hereinabove with reference to FIGS. 1-8.

VGAM193 gene, herein designated VGAM GENE, is a viral gene contained in the genome of Vaccinia Virus. VGAM193 host target gene, herein designated VGAM HOST TARGET GENE, is a human gene contained in the human genome.

VGAM193 gene encodes a VGAM193 precursor RNA, herein designated VGAM PRECURSOR RNA. Similar to other miRNA genes, and unlike most ordinary genes, VGAM193 precursor RNA does not encode a protein. A nucleotide sequence identical or highly similar to the nucleotide sequence of VGAM193 precursor RNA is designated SEQ ID:179, and is provided hereinbelow with reference to the sequence listing part. Nucleotide sequence SEQ ID:179 is located at position 103285 relative to the genome of Vaccinia Virus.

VGAM193 precursor RNA folds onto itself, forming VGAM193 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM193 folded precursor RNA into VGAM193 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM193 RNA is designated SEQ ID:528, and is provided hereinbelow with reference to the sequence listing part.

VGAM193 host target gene, herein designated VGAM HOST TARGET GENE, encodes a corresponding messenger RNA, VGAM193 host target RNA, herein designated VGAM HOST TARGET RNA. VGAM193 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM193 RNA, herein designated VGAM RNA, binds complementarily to one or more host target binding sites located in untranslated regions of VGAM193 host target RNA, herein designated VGAM HOST TARGET RNA. This complementary binding is due to the fact that the nucleotide sequence of VGAM193 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM193 RNA, herein designated VGAM RNA, may have a different number of host target binding sites in untranslated regions of a VGAM193 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM193 RNA, herein designated VGAM RNA, to host target binding sites on VGAM193 host target RNA, herein designated VGAM HOST TARGET RNA, such as BINDING SITE I, BINDING SITE II and BINDING SITE III, inhibits translation of VGAM193 host target RNA into VGAM193 host target protein, herein designated VGAM HOST TARGET PROTEIN. VGAM host target protein is therefore outlined by a broken line.

It is appreciated that VGAM193 host target gene, herein designated VGAM HOST TARGET GENE, in fact represents a plurality of VGAM193 host target genes. The mRNA of each one of this plurality of VGAM193 host target genes comprises one or more host target binding sites, each having a nucleotide sequence which is at least partly complementary to VGAM193 RNA, herein designated VGAM RNA, and which when bound by VGAM193 RNA causes inhibition of translation of respective one or more VGAM193 host target proteins.

It is further appreciated by one skilled in the art that the mode of translational inhibition illustrated by FIG. 1 with specific reference to translational inhibition exerted by VGAM193 gene, herein designated VGAM GENE, on one or more VGAM193 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM193 is inhibition of expression of host target genes, as part of a novel viral mechanism of attacking a host. Accordingly, utilities of VGAM193 include diagnosis, prevention and treatment of viral infection by Vaccinia Virus. Specific functions, and accordingly utilities, of VGAM193 correlate with, and may be deduced from, the identity of the host target genes which VGAM193 binds and inhibits, and the function of these host target genes, as elaborated hereinbelow.

Nucleotide sequences of the VGAM193 precursor RNA, herein designated VGAM PRECURSOR RNA, and of the ‘diced’ VGAM193 RNA, herein designated VGAM RNA, and a schematic representation of the secondary folding of VGAM193 folded precursor RNA, herein designated VGAM FOLDED PRECURSOR RNA, of VGAM193 are further described hereinbelow with reference to Table 1.

Nucleotide sequences of host target binding sites, such as BINDING SITE-I, BINDING SITE-II and BINDING SITE-III of FIG. 1, found on VGAM193 host target RNA, and schematic representation of the complementarity of each of these host target binding sites to VGAM193 RNA, herein designated VGAM RNA, are described hereinbelow with reference to Table 2.

As mentioned hereinabove with reference to FIG. 1, a function of VGAM193 gene, herein designated VGAM is inhibition of expression of VGAM193 target genes. It is appreciated that specific functions, and accordingly utilities, of VGAM193 correlate with, and may be deduced from, the identity of the target genes which VGAM193 binds and inhibits, and the function of these target genes, as elaborated hereinbelow.

KIAA1228 (Accession XM_{—}036408) is a VGAM193 host target gene. KIAA1228 BINDING SITE is HOST TARGET binding site found in the 3′ untranslated region of mRNA encoded by KIAA1228, corresponding to a HOST TARGET binding site such as BINDING SITE I, BINDING SITE II or BINDING SITE III. Table 2 illustrates the complementarity of the nucleotide sequences of KIAA1228 BINDING SITE, designated SEQ ID:2712, to the nucleotide sequence of VGAM193 RNA, herein designated VGAM RNA, also designated SEQ ID:528.

A function of VGAM193 is therefore inhibition of KIAA1228 (Accession XM_{—}036408). Accordingly, utilities of VGAM193 include diagnosis, prevention and treatment of diseases and clinical conditions associated with KIAA1228. KIAA1655 (Accession XM_{—}039442) is another VGAM193 host target gene. KIAA1655 BINDING SITE is HOST TARGET binding site found in the 5′ untranslated region of mRNA encoded by KIAA1655, corresponding to a HOST TARGET binding site such as BINDING SITE I, BINDING SITE II or BINDING SITE III. Table 2 illustrates the complementarity of the nucleotide sequences of KIAA1655 BINDING SITE, designated SEQ ID:2754, to the nucleotide sequence of VGAM193 RNA, herein designated VGAM RNA, also designated SEQ ID:528.

Another function of VGAM193 is therefore inhibition of KIAA1655 (Accession XM_{—}039442). Accordingly, utilities of VGAM193 include diagnosis, prevention and treatment of diseases and clinical conditions associated with KIAA1655. N-deacetylase/N-sulfotransferase (heparan glucosaminyl) 3 (NDST3, Accession NM_{—}004784) is another VGAM193 host target gene. NDST3 BINDING SITE is HOST TARGET binding site found in the 3′ untranslated region of mRNA encoded by NDST3, corresponding to a HOST TARGET binding site such as BINDING SITE I, BINDING SITE II or BINDING SITE III. Table 2 illustrates the complementarity of the nucleotide sequences of NDST3 BINDING SITE, designated SEQ ID:1157, to the nucleotide sequence of VGAM193 RNA, herein designated VGAM RNA, also designated SEQ ID:528.

Another function of VGAM193 is therefore inhibition of N-deacetylase/N-sulfotransferase (heparan glucosaminyl) 3 (NDST3, Accession NM_{—}004784). Accordingly, utilities of VGAM193 include diagnosis, prevention and treatment of diseases and clinical conditions associated with NDST3. PP1057 (Accession NM_{—}031285) is another VGAM193 host target gene. PP1057 BINDING SITE is HOST TARGET binding site found in the 3′ untranslated region of mRNA encoded by PP1057, corresponding to a HOST TARGET binding site such as BINDING SITE I, BINDING SITE II or BINDING SITE III. Table 2 illustrates the complementarity of the nucleotide sequences of PP1057 BINDING SITE, designated SEQ ID:2189, to the nucleotide sequence of VGAM193 RNA, herein designated VGAM RNA, also designated SEQ ID:528.

Another function of VGAM193 is therefore inhibition of PP1057 (Accession NM_{—}031285). Accordingly, utilities of VGAM193 include diagnosis, prevention and treatment of diseases and clinical conditions associated with PP1057. RNP24 (Accession NM_{—}006815) is another VGAM193 host target gene. RNP24 BINDING SITE is HOST TARGET binding site found in the 3′ untranslated region of mRNA encoded by RNP24, corresponding to a HOST TARGET binding site such as BINDING SITE I, BINDING SITE II or BINDING SITE III. Table 2 illustrates the complementarity of the nucleotide sequences of RNP24 BINDING SITE, designated SEQ ID:1333, to the nucleotide sequence of VGAM193 RNA, herein designated VGAM RNA, also designated SEQ ID:528.

Another function of VGAM193 is therefore inhibition of RNP24 (Accession NM_{—}006815). Accordingly, utilities of VGAM193 include diagnosis, prevention and treatment of diseases and clinical conditions associated with RNP24. LOC150142 (Accession XM_{—}086791) is another VGAM193 host target gene. LOC150142 BINDING SITE is HOST TARGET binding site found in the 5′ untranslated region of mRNA encoded by LOC150142, corresponding to a HOST TARGET binding site such as BINDING SITE I, BINDING SITE II or BINDING SITE III. Table 2 illustrates the complementarity of the nucleotide sequences of LOC150142 BINDING SITE, designated SEQ ID:3142, to the nucleotide sequence of VGAM193 RNA, herein designated VGAM RNA, also designated SEQ ID:528.

Another function of VGAM193 is therefore inhibition of LOC150142 (Accession XM_{—}086791). Accordingly, utilities of VGAM193 include diagnosis, prevention and treatment of diseases and clinical conditions associated with LOC150142. LOC199899 (Accession XM_{—}117153) is another VGAM193 host target gene. LOC199899 BINDING SITE is HOST TARGET binding site found in the 5′ untranslated region of mRNA encoded by LOC199899, corresponding to a HOST TARGET binding site such as BINDING SITE I, BINDING SITE II or BINDING SITE III. Table 2 illustrates the complementarity of the nucleotide sequences of LOC199899 BINDING SITE, designated SEQ ID:3473, to the nucleotide sequence of VGAM193 RNA, herein designated VGAM RNA, also designated SEQ ID:528.

Another function of VGAM193 is therefore inhibition of LOC199899 (Accession XM_{—}117153). Accordingly, utilities of VGAM193 include diagnosis, prevention and treatment of diseases and clinical conditions associated with LOC199899. LOC222068 (Accession XM_{—}166556) is another VGAM193 host target gene. LOC222068 BINDING SITE is HOST TARGET binding site found in the 3′ untranslated region of mRNA encoded by LOC222068, corresponding to a HOST TARGET binding site such as BINDING SITE I, BINDING SITE II or BINDING SITE III. Table 2 illustrates the complementarity of the nucleotide sequences of LOC222068 BINDING SITE, designated SEQ ID:3573, to the nucleotide sequence of VGAM193 RNA, herein designated VGAM RNA, also designated SEQ ID:528.

Another function of VGAM193 is therefore inhibition of LOC222068 (Accession XM_{—}166556). Accordingly, utilities of VGAM193 include diagnosis, prevention and treatment of diseases and clinical conditions associated with LOC222068. FIG. 1 further provides a conceptual description of a novel bioinformatically detected viral gene of the present invention, referred to here as Viral Genomic Address Messenger 194 (VGAM194) viral gene, which modulates expression of respective host target genes thereof, the function and utility of which host target genes is known in the art.

VGAM194 is a novel bioinformatically detected regulatory, non protein coding, viral micro RNA (miRNA) gene. The method by which VGAM194 was detected is described hereinabove with reference to FIGS. 1-8.

VGAM194 gene, herein designated VGAM GENE, is a viral gene contained in the genome of Vaccinia Virus. VGAM194 host target gene, herein designated VGAM HOST TARGET GENE, is a human gene contained in the human genome.

VGAM194 gene encodes a VGAM194 precursor RNA, herein designated VGAM PRECURSOR RNA. Similar to other miRNA genes, and unlike most ordinary genes, VGAM194 precursor RNA does not encode a protein. A nucleotide sequence identical or highly similar to the nucleotide sequence of VGAM194 precursor RNA is designated SEQ ID:180, and is provided hereinbelow with reference to the sequence listing part. Nucleotide sequence SEQ ID:180 is located at position 103379 relative to the genome of Vaccinia Virus.

VGAM194 precursor RNA folds onto itself, forming VGAM194 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM194 folded precursor RNA into VGAM194 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM194 RNA is designated SEQ ID:529, and is provided hereinbelow with reference to the sequence listing part.

VGAM194 host target gene, herein designated VGAM HOST TARGET GENE, encodes a corresponding messenger RNA, VGAM194 host target RNA, herein designated VGAM HOST TARGET RNA. VGAM194 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM194 RNA, herein designated VGAM RNA, binds complementarily to one or more host target binding sites located in untranslated regions of VGAM194 host target RNA, herein designated VGAM HOST TARGET RNA. This complementary binding is due to the fact that the nucleotide sequence of VGAM194 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM194 RNA, herein designated VGAM RNA, may have a different number of host target binding sites in untranslated regions of a VGAM194 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM194 RNA, herein designated VGAM RNA, to host target binding sites on VGAM194 host target RNA, herein designated VGAM HOST TARGET RNA, such as BINDING SITE I, BINDING SITE II and BINDING SITE III, inhibits translation of VGAM194 host target RNA into VGAM194 host target protein, herein designated VGAM HOST TARGET PROTEIN. VGAM host target protein is therefore outlined by a broken line.

It is appreciated that VGAM194 host target gene, herein designated VGAM HOST TARGET GENE, in fact represents a plurality of VGAM194 host target genes. The mRNA of each one of this plurality of VGAM194 host target genes comprises one or more host target binding sites, each having a nucleotide sequence which is at least partly complementary to VGAM194 RNA, herein designated VGAM RNA, and which when bound by VGAM194 RNA causes inhibition of translation of respective one or more VGAM194 host target proteins.

It is further appreciated by one skilled in the art that the mode of translational inhibition illustrated by FIG. 1 with specific reference to translational inhibition exerted by VGAM194 gene, herein designated VGAM GENE, on one or more VGAM194 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM194 is inhibition of expression of host target genes, as part of a novel viral mechanism of attacking a host. Accordingly, utilities of VGAM194 include diagnosis, prevention and treatment of viral infection by Vaccinia Virus. Specific functions, and accordingly utilities, of VGAM194 correlate with, and may be deduced from, the identity of the host target genes which VGAM194 binds and inhibits, and the function of these host target genes, as elaborated hereinbelow.

Nucleotide sequences of the VGAM194 precursor RNA, herein designated VGAM PRECURSOR RNA, and of the ‘diced’ VGAM194 RNA, herein designated VGAM RNA, and a schematic representation of the secondary folding of VGAM194 folded precursor RNA, herein designated VGAM FOLDED PRECURSOR RNA, of VGAM194 are further described hereinbelow with reference to Table 1.

Nucleotide sequences of host target binding sites, such as BINDING SITE-I, BINDING SITE-II and BINDING SITE-III of FIG. 1, found on VGAM194 host target RNA, and schematic representation of the complementarity of each of these host target binding sites to VGAM194 RNA, herein designated VGAM RNA, are described hereinbelow with reference to Table 2.

As mentioned hereinabove with reference to FIG. 1, a function of VGAM194 gene, herein designated VGAM is inhibition of expression of VGAM194 target genes. It is appreciated that specific functions, and accordingly utilities, of VGAM194 correlate with, and may be deduced from, the identity of the target genes which VGAM194 binds and inhibits, and the function of these target genes, as elaborated hereinbelow.

HUS1 Checkpoint Homolog (S. pombe) (HUS1, Accession XM_{—}165873) is a VGAM194 host target gene. HUS1 BINDING SITE is HOST TARGET binding site found in the 3′ untranslated region of mRNA encoded by HUS1, corresponding to a HOST TARGET binding site such as BINDING SITE I, BINDING SITE II or BINDING SITE III. Table 2 illustrates the complementarity of the nucleotide sequences of HUS1 BINDING SITE, designated SEQ ID:3509, to the nucleotide sequence of VGAM194 RNA, herein designated VGAM RNA, also designated SEQ ID:529.

A function of VGAM194 is therefore inhibition of HUS1 Checkpoint Homolog (S. pombe) (HUS1, Accession XM_{—}165873), a gene which May form DNA damage-responsive protein complex. Accordingly, utilities of VGAM194 include diagnosis, prevention and treatment of diseases and clinical conditions associated with HUS1. The function of HUS1 has been established by previous studies. The S. pombe ‘checkpoint rad’ genes hus1, rad1 (OMIM Ref. No. 603153), rad3, rad9 (OMIM Ref. No. 603761), rad17 (OMIM Ref. No. 603139), and rad26 are essential for both the incomplete DNA replication (S-M) and DNA damage checkpoints. An early step in the DNA damage checkpoint response appears to involve activation of the rad3 phosphatidylinositol 3-kinase-related (PIK-R) checkpoint kinase (see OMIM Ref. No. AT; 208900) by the other 5 checkpoint rad gene products. Kostrub et al. (1998) found that the fission yeast hus1 and rad1 proteins form a stable complex, and that the formation of this complex is dependent on rad9, suggesting that these 3 proteins may exist in a discrete complex in the absence of checkpoint activation. Hus1 is phosphorylated in response to DNA damage, and this phosphorylation requires rad3 and the other checkpoint rad genes. By searching EST databases, Kostrub et al. (1998) and Dean et al. (1998) each identified mouse and human cDNAs encoding hus1 homologs. Kostrub et al. (1998) reported that the predicted 281-amino acid human protein shares 30% and 86% identity with S. pombe hus1 and mouse Hus1, respectively. However, neither mammalian gene complemented a fission yeast hus1 mutation. Volkmer and Karnitz (1999) demonstrated that the human RAD1 and HUS1 proteins associate in a complex that interacts with a highly modified form of RAD9. They concluded that these 3 proteins are central components of a DNA damage-responsive protein complex in human cells. AU-rich elements (AREs) are cis-acting sequences typically found in 3-prime untranslated regions of many labile mRNAs. AREs either mediate rapid degradation of mRNA or inhibit its translation. Dominguez et al. (1998) identified EE2-16C, a HUS1 cDNA, among a collection of ARE-containing mRNAs.

Full details of the abovementioned studies are described in the following publications, the disclosure of which are hereby incorporated by reference:
Kostrub, C. F.; Knudsen, K.; Subramani, S.; Enoch, T.: Hus1p, a conserved fission yeast checkpoint protein, interacts with Rad1p and is phosphorylated in response to DNA damage. EMBO J. 17: 2055-2066, 1998.; and
Volkmer, E.; Karnitz, L. M.: Human homologs of Schizosaccharomyces pombe Rad1, Hus1, and Rad9 form a DNA damage-responsive protein complex. J. Biol. Chem. 274: 567-570, 1999.

Further studies establishing the function and utilities of HUS1 are found in John Hopkins OMIM database record ID 603760, and in sited publications numbered 145, 1744-174 and 580 listed in the bibliography section hereinbelow, which are also hereby incorporated by reference. Transient Receptor Potential Cation Channel, Sub family M, Member 8 (TRPM8, Accession NM_{—}024080) is another VGAM194 host target gene. TRPM8 BINDING SITE is HOST TARGET binding site found in the 3′ untranslated region of mRNA encoded by TRPM8, corresponding to a HOST TARGET binding site such as BINDING SITE I, BINDING SITE II or BINDING SITE III. Table 2 illustrates the complementarity of the nucleotide sequences of TRPM8 BINDING SITE, designated SEQ ID:2052, to the nucleotide sequence of VGAM194 RNA, herein designated VGAM RNA, also designated SEQ ID:529.

Another function of VGAM194 is therefore inhibition of Transient Receptor Potential Cation Channel, Sub family M, Member 8 (TRPM8, Accession NM_{—}024080), a gene which is thought to form a receptor-activated calcium permeant cation channel. Accordingly, utilities of VGAM194 include diagnosis, prevention and treatment of diseases and clinical conditions associated with TRPM8. The function of TRPM8 has been established by previous studies. Using expression cloning of a rat trigeminal nerve cDNA library in a human embryonic kidney cell line and screening for changes in intracellular calcium on exposure to room-temperature menthol, McKemy et al. (2002) identified a cDNA encoding Cmr1 (cold-menthol receptor-1). The deduced 1,104-amino acid protein, 92% identical to human TRPM8, is also responsive to icilin, cold (with a range from 8 to 28 degrees C.), and eucalyptol (the main constituent of oil of Eucalyptus) with low or no responses to menthone, camphor, cyclohexanol, or capsaicin, the agonist for VR1, which is related to the TRP family. Northern blot analysis detected transcripts of 6.0 and 4.5 kb in rat dorsal root ganglia and trigeminal neurons. In situ hybridization analysis demonstrated expression in small-diameter, but not larger-diameter, sensory neurons, similar in size to VR1-expressing cells. Cells expressing both Cmr1 and Vr1 endow cells to respond to distinct temperature thresholds, cool and hot (more than 43 degrees C.), respectively. McKemy et al. (2002) suggested this coexpression may explain the paradox that noxious cold is sometimes perceived as burning pain. The authors also proposed that in other contexts, such as prostate and tumors, an endogenous menthol-like ligand may modulate the TRPM8 channel. Peier et al. (2002) showed that mouse Trpm8 is specifically expressed in a subset of pain- and temperature-sensing neurons. Cells overexpressing the Trpm8 channel could be activated by cold temperatures and by a cooling agent, menthol. The authors concluded that the identification of a cold-sensing TRP channel in a distinct subpopulation of sensory neurons implicated an expanded role for this family of ion channels in somatic sensory detection.

Full details of the abovementioned studies are described in the following publications, the disclosure of which are hereby incorporated by reference:
McKemy, D. D.; Neuhausser, W. M.; Julius, D.: Identification of a cold receptor reveals a general role for TRP channels in thermosensation. Nature 416: 52-58, 2002.; and
Peier, A. M.; Moqrich, A.; Hergarden, A. C.; Reeve, A. J.; Andersson, D. A.; Story, G. M.; Earley, T. J.; Dragoni, I.; McIntyre, P.; Bevan, S.; Patapoutian, A.: A TRP channel that sense.

Further studies establishing the function and utilities of TRPM8 are found in John Hopkins OMIM database record ID 606678, and in sited publications numbered 1268-127 and 1113 listed in the bibliography section hereinbelow, which are also hereby incorporated by reference. LOC148936 (Accession XM_{—}097556) is another VGAM194 host target gene. LOC148936 BINDING SITE is HOST TARGET binding site found in the 3′ untranslated region of mRNA encoded by LOC148936, corresponding to a HOST TARGET binding site such as BINDING SITE I, BINDING SITE II or BINDING SITE III. Table 2 illustrates the complementarity of the nucleotide sequences of LOC148936 BINDING SITE, designated SEQ ID:3304, to the nucleotide sequence of VGAM194 RNA, herein designated VGAM RNA, also designated SEQ ID:529.

Another function of VGAM194 is therefore inhibition of LOC148936 (Accession XM_{—}097556). Accordingly, utilities of VGAM194 include diagnosis, prevention and treatment of diseases and clinical conditions associated with LOC148936. LOC148938 (Accession XM_{—}097555) is another VGAM194 host target gene. LOC148938 BINDING SITE is HOST TARGET binding site found in the 3′ untranslated region of mRNA encoded by LOC148938, corresponding to a HOST TARGET binding site such as BINDING SITE I, BINDING SITE II or BINDING SITE III. Table 2 illustrates the complementarity of the nucleotide sequences of LOC148938 BINDING SITE, designated SEQ ID:3303, to the nucleotide sequence of VGAM194 RNA, herein designated VGAM RNA, also designated SEQ ID:529.

Another function of VGAM194 is therefore inhibition of LOC148938 (Accession XM_{—}097555). Accordingly, utilities of VGAM194 include diagnosis, prevention and treatment of diseases and clinical conditions associated with LOC148938. LOC200803 (Accession XM_{—}114299) is another VGAM194 host target gene. LOC200803 BINDING SITE is HOST TARGET binding site found in the 3′ untranslated region of mRNA encoded by LOC200803, corresponding to a HOST TARGET binding site such as BINDING SITE I, BINDING SITE II or BINDING SITE III. Table 2 illustrates the complementarity of the nucleotide sequences of LOC200803 BINDING SITE, designated SEQ ID:3452, to the nucleotide sequence of VGAM194 RNA, herein designated VGAM RNA, also designated SEQ ID:529.

Another function of VGAM194 is therefore inhibition of LOC200803 (Accession XM_{—}114299). Accordingly, utilities of VGAM194 include diagnosis, prevention and treatment of diseases and clinical conditions associated with LOC200803. LOC255332 (Accession XM_{—}171235) is another VGAM194 host target gene. LOC255332 BINDING SITE is HOST TARGET binding site found in the 5′ untranslated region of mRNA encoded by LOC255332, corresponding to a HOST TARGET binding site such as BINDING SITE I, BINDING SITE II or BINDING SITE III. Table 2 illustrates the complementarity of the nucleotide sequences of LOC255332 BINDING SITE, designated SEQ ID:3696, to the nucleotide sequence of VGAM194 RNA, herein designated VGAM RNA, also designated SEQ ID:529.

Another function of VGAM194 is therefore inhibition of LOC255332 (Accession XM_{—}171235). Accordingly, utilities of VGAM194 include diagnosis, prevention and treatment of diseases and clinical conditions associated with LOC255332. LOC90459 (Accession XM_{—}031826) is another VGAM194 host target gene. LOC90459 BINDING SITE is HOST TARGET binding site found in the 3′ untranslated region of mRNA encoded by LOC90459, corresponding to a HOST TARGET binding site such as BINDING SITE I, BINDING SITE II or BINDING SITE III. Table 2 illustrates the complementarity of the nucleotide sequences of LOC90459 BINDING SITE, designated SEQ ID:2641, to the nucleotide sequence of VGAM194 RNA, herein designated VGAM RNA, also designated SEQ ID:529.

Another function of VGAM194 is therefore inhibition of LOC90459 (Accession XM_{—}031826). Accordingly, utilities of VGAM194 include diagnosis, prevention and treatment of diseases and clinical conditions associated with LOC90459. FIG. 1 further provides a conceptual description of a novel bioinformatically detected viral gene of the present invention, referred to here as Viral Genomic Address Messenger 195 (VGAM195) viral gene, which modulates expression of respective host target genes thereof, the function and utility of which host target genes is known in the art.

VGAM195 is a novel bioinformatically detected regulatory, non protein coding, viral micro RNA (miRNA) gene. The method by which VGAM195 was detected is described hereinabove with reference to FIGS. 1-8.

VGAM195 gene, herein designated VGAM GENE, is a viral gene contained in the genome of Vaccinia Virus. VGAM195 host target gene, herein designated VGAM HOST TARGET GENE, is a human gene contained in the human genome.

VGAM195 gene encodes a VGAM195 precursor RNA, herein designated VGAM PRECURSOR RNA. Similar to other miRNA genes, and unlike most ordinary genes, VGAM195 precursor RNA does not encode a protein. A nucleotide sequence identical or highly similar to the nucleotide sequence of VGAM195 precursor RNA is designated SEQ ID:181, and is provided hereinbelow with reference to the sequence listing part. Nucleotide sequence SEQ ID:181 is located at position 105936 relative to the genome of Vaccinia Virus.

VGAM195 precursor RNA folds onto itself, forming VGAM195 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM195 folded precursor RNA into VGAM195 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM195 RNA is designated SEQ ID:530, and is provided hereinbelow with reference to the sequence listing part.

VGAM195 host target gene, herein designated VGAM HOST TARGET GENE, encodes a corresponding messenger RNA, VGAM195 host target RNA, herein designated VGAM HOST TARGET RNA. VGAM195 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM195 RNA, herein designated VGAM RNA, binds complementarily to one or more host target binding sites located in untranslated regions of VGAM195 host target RNA, herein designated VGAM HOST TARGET RNA. This complementary binding is due to the fact that the nucleotide sequence of VGAM195 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM195 RNA, herein designated VGAM RNA, may have a different number of host target binding sites in untranslated regions of a VGAM195 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM195 RNA, herein designated VGAM RNA, to host target binding sites on VGAM195 host target RNA, herein designated VGAM HOST TARGET RNA, such as BINDING SITE I, BINDING SITE II and BINDING SITE III, inhibits translation of VGAM195 host target RNA into VGAM195 host target protein, herein designated VGAM HOST TARGET PROTEIN. VGAM host target protein is therefore outlined by a broken line.

It is appreciated that VGAM195 host target gene, herein designated VGAM HOST TARGET GENE, in fact represents a plurality of VGAM195 host target genes. The mRNA of each one of this plurality of VGAM195 host target genes comprises one or more host target binding sites, each having a nucleotide sequence which is at least partly complementary to VGAM195 RNA, herein designated VGAM RNA, and which when bound by VGAM195 RNA causes inhibition of translation of respective one or more VGAM195 host target proteins.

It is further appreciated by one skilled in the art that the mode of translational inhibition illustrated by FIG. 1 with specific reference to translational inhibition exerted by VGAM195 gene, herein designated VGAM GENE, on one or more VGAM195 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM195 is inhibition of expression of host target genes, as part of a novel viral mechanism of attacking a host. Accordingly, utilities of VGAM195 include diagnosis, prevention and treatment of viral infection by Vaccinia Virus. Specific functions, and accordingly utilities, of VGAM195 correlate with, and may be deduced from, the identity of the host target genes which VGAM195 binds and inhibits, and the function of these host target genes, as elaborated hereinbelow.

Nucleotide sequences of the VGAM195 precursor RNA, herein designated VGAM PRECURSOR RNA, and of the ‘diced’ VGAM195 RNA, herein designated VGAM RNA, and a schematic representation of the secondary folding of VGAM195 folded precursor RNA, herein designated VGAM FOLDED PRECURSOR RNA, of VGAM195 are further described hereinbelow with reference to Table 1.

Nucleotide sequences of host target binding sites, such as BINDING SITE-I, BINDING SITE-II and BINDING SITE-III of FIG. 1, found on VGAM195 host target RNA, and schematic representation of the complementarity of each of these host target binding sites to VGAM195 RNA, herein designated VGAM RNA, are described hereinbelow with reference to Table 2.

As mentioned hereinabove with reference to FIG. 1, a function of VGAM195 gene, herein designated VGAM is inhibition of expression of VGAM195 target genes. It is appreciated that specific functions, and accordingly utilities, of VGAM195 correlate with, and may be deduced from, the identity of the target genes which VGAM195 binds and inhibits, and the function of these target genes, as elaborated hereinbelow.

DKFZp434E0519 (Accession NM_{—}032247) is a VGAM195 host target gene. DKFZp434E0519 BINDING SITE is HOST TARGET binding site found in the 5′ untranslated region of mRNA encoded by DKFZp434E0519, corresponding to a HOST TARGET binding site such as BINDING SITE I, BINDING SITE II or BINDING SITE III. Table 2 illustrates the complementarity of the nucleotide sequences of DKFZp434E0519 BINDING SITE, designated SEQ ID:2241, to the nucleotide sequence of VGAM195 RNA, herein designated VGAM RNA, also designated SEQ ID:530.

A function of VGAM195 is therefore inhibition of DKFZp434E0519 (Accession NM_{—}032247). Accordingly, utilities of VGAM195 include diagnosis, prevention and treatment of diseases and clinical conditions associated with DKFZp434E0519. HSU84971 (Accession NM_{—}013303) is another VGAM195 host target gene. HSU84971 BINDING SITE is HOST TARGET binding site found in the 3′ untranslated region of mRNA encoded by HSU84971, corresponding to a HOST TARGET binding site such as BINDING SITE I, BINDING SITE II or BINDING SITE III. Table 2 illustrates the complementarity of the nucleotide sequences of HSU84971 BINDING SITE, designated SEQ ID: 1439, to the nucleotide sequence of VGAM195 RNA, herein designated VGAM RNA, also designated SEQ ID:530.

Another function of VGAM195 is therefore inhibition of HSU84971 (Accession NM_{—}013303). Accordingly, utilities of VGAM195 include diagnosis, prevention and treatment of diseases and clinical conditions associated with HSU84971. LOC157663 (Accession XM_{—}088354) is another VGAM195 host target gene. LOC157663 BINDING SITE is HOST TARGET binding site found in the 3′ untranslated region of mRNA encoded by LOC157663, corresponding to a HOST TARGET binding site such as BINDING SITE I, BINDING SITE II or BINDING SITE III. Table 2 illustrates the complementarity of the nucleotide sequences of LOC157663 BINDING SITE, designated SEQ ID:3203, to the nucleotide sequence of VGAM195 RNA, herein designated VGAM RNA, also designated SEQ ID:530.

Another function of VGAM195 is therefore inhibition of LOC157663 (Accession XM_{—}088354). Accordingly, utilities of VGAM195 include diagnosis, prevention and treatment of diseases and clinical conditions associated with LOC157663. FIG. 1 further provides a conceptual description of a novel bioinformatically detected viral gene of the present invention, referred to here as Viral Genomic Address Messenger 196 (VGAM196) viral gene, which modulates expression of respective host target genes thereof, the function and utility of which host target genes is known in the art.

VGAM196 is a novel bioinformatically detected regulatory, non protein coding, viral micro RNA (miRNA) gene. The method by which VGAM196 was detected is described hereinabove with reference to FIGS. 1-8.

VGAM196 gene, herein designated VGAM GENE, is a viral gene contained in the genome of Vaccinia Virus. VGAM196 host target gene, herein designated VGAM HOST TARGET GENE, is a human gene contained in the human genome.

VGAM196 gene encodes a VGAM196 precursor RNA, herein designated VGAM PRECURSOR RNA. Similar to other miRNA genes, and unlike most ordinary genes, VGAM196 precursor RNA does not encode a protein. A nucleotide sequence identical or highly similar to the nucleotide sequence of VGAM196 precursor RNA is designated SEQ ID:182, and is provided hereinbelow with reference to the sequence listing part. Nucleotide sequence SEQ ID:182 is located at position 103914 relative to the genome of Vaccinia Virus.

VGAM196 precursor RNA folds onto itself, forming VGAM196 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM196 folded precursor RNA into VGAM196 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM196 RNA is designated SEQ ID:531, and is provided hereinbelow with reference to the sequence listing part.

VGAM196 host target gene, herein designated VGAM HOST TARGET GENE, encodes a corresponding messenger RNA, VGAM196 host target RNA, herein designated VGAM HOST TARGET RNA. VGAM196 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM196 RNA, herein designated VGAM RNA, binds complementarily to one or more host target binding sites located in untranslated regions of VGAM196 host target RNA, herein designated VGAM HOST TARGET RNA. This complementary binding is due to the fact that the nucleotide sequence of VGAM196 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM196 RNA, herein designated VGAM RNA, may have a different number of host target binding sites in untranslated regions of a VGAM196 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM196 RNA, herein designated VGAM RNA, to host target binding sites on VGAM196 host target RNA, herein designated VGAM HOST TARGET RNA, such as BINDING SITE I, BINDING SITE II and BINDING SITE III, inhibits translation of VGAM196 host target RNA into VGAM196 host target protein, herein designated VGAM HOST TARGET PROTEIN. VGAM host target protein is therefore outlined by a broken line.

It is appreciated that VGAM196 host target gene, herein designated VGAM HOST TARGET GENE, in fact represents a plurality of VGAM196 host target genes. The mRNA of each one of this plurality of VGAM196 host target genes comprises one or more host target binding sites, each having a nucleotide sequence which is at least partly complementary to VGAM196 RNA, herein designated VGAM RNA, and which when bound by VGAM196 RNA causes inhibition of translation of respective one or more VGAM196 host target proteins.

It is further appreciated by one skilled in the art that the mode of translational inhibition illustrated by FIG. 1 with specific reference to translational inhibition exerted by VGAM196 gene, herein designated VGAM GENE, on one or more VGAM196 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM196 is inhibition of expression of host target genes, as part of a novel viral mechanism of attacking a host. Accordingly, utilities of VGAM196 include diagnosis, prevention and treatment of viral infection by Vaccinia Virus. Specific functions, and accordingly utilities, of VGAM196 correlate with, and may be deduced from, the identity of the host target genes which VGAM196 binds and inhibits, and the function of these host target genes, as elaborated hereinbelow.

Nucleotide sequences of the VGAM196 precursor RNA, herein designated VGAM PRECURSOR RNA, and of the ‘diced’ VGAM196 RNA, herein designated VGAM RNA, and a schematic representation of the secondary folding of VGAM196 folded precursor RNA, herein designated VGAM FOLDED PRECURSOR RNA, of VGAM196 are further described hereinbelow with reference to Table 1.

Nucleotide sequences of host target binding sites, such as BINDING SITE-I, BINDING SITE-II and BINDING SITE-III of FIG. 1, found on VGAM196 host target RNA, and schematic representation of the complementarity of each of these host target binding sites to VGAM196 RNA, herein designated VGAM RNA, are described hereinbelow with reference to Table 2.

As mentioned hereinabove with reference to FIG. 1, a function of VGAM196 gene, herein designated VGAM is inhibition of expression of VGAM196 target genes. It is appreciated that specific functions, and accordingly utilities, of VGAM196 correlate with, and may be deduced from, the identity of the target genes which VGAM196 binds and inhibits, and the function of these target genes, as elaborated hereinbelow.

Exostoses (multiple) 2 (EXT2, Accession NM_{—}000401) is a VGAM196 host target gene. EXT2 BINDING SITE is HOST TARGET binding site found in the 3′ untranslated region of mRNA encoded by EXT2, corresponding to a HOST TARGET binding site such as BINDING SITE I, BINDING SITE II or BINDING SITE III. Table 2 illustrates the complementarity of the nucleotide sequences of EXT2 BINDING SITE, designated SEQ ID:737, to the nucleotide sequence of VGAM196 RNA, herein designated VGAM RNA, also designated SEQ ID:531.

A function of VGAM196 is therefore inhibition of Exostoses (multiple) 2 (EXT2, Accession NM_{—}000401). Accordingly, utilities of VGAM196 include diagnosis, prevention and treatment of diseases and clinical conditions associated with EXT2. Muscleblind-like (Drosophila) (MBNL, Accession NM_{—}021038) is another VGAM196 host target gene. MBNL BINDING SITE is HOST TARGET binding site found in the 3′ untranslated region of mRNA encoded by MBNL, corresponding to a HOST TARGET binding site such as BINDING SITE I, BINDING SITE II or BINDING SITE III. Table 2 illustrates the complementarity of the nucleotide sequences of MBNL BINDING SITE, designated SEQ ID:1936, to the nucleotide sequence of VGAM196 RNA, herein designated VGAM RNA, also designated SEQ ID:531.

Another function of VGAM196 is therefore inhibition of Muscleblind-like (Drosophila) (MBNL, Accession NM_{—}021038), a gene which binds to cug triplet repeat expansion dsrna (by similarity). Accordingly, utilities of VGAM196 include diagnosis, prevention and treatment of diseases and clinical conditions associated with MBNL. The function of MBNL has been established by previous studies. By screening for cDNAs with the potential to encode large proteins expressed in brain, Ishikawa et al. (1997) identified a cDNA encoding MBNL, which they designated KIAA0428. KIAA0428 encodes a deduced 370-amino acid protein. RT-PCR analysis detected highest expression of KIAA0428 in skeletal muscle, followed by prostate, lung, heart, small intestine, ovary, and placenta. Triplet repeat expansion disorders (e.g., myotonic dystrophy; 160900) are characterized by genetic anticipation in which disease severity is proportional and age-of-onset is inversely proportional to the size of the expansion mutation. By biochemical purification of HeLa cell proteins binding to dystrophia myotonica (DM1) protein kinase (DMPK; 605377) RNAs with variable numbers of CUG repeats, followed by peptide sequence analysis and PCR, Miller et al. (2000) isolated cDNAs encoding isoforms of MBNL, which they termed EXP. The 42- and 40-kD isoforms, EXP42 and EXP40, are identical to a previously identified 388-amino acid MBNL protein (GenBank CAA74155) and KIAA0428, respectively, while the 35-kD isoform, EXP35, is a novel 305-amino acid protein. Northern blot analysis revealed 6.5- and 5.3-kb EXP transcripts that were highly expressed in cardiac and skeletal muscle. Western blot analysis showed high expression of EXP42 in HeLa and lymphoblastoid cell lines. Immunofluorescence microscopy demonstrated nuclear and cytoplasmic expression of EXP42 in normal myoblasts, while nuclear foci were enriched in DM1 myoblasts. FISH and immunofluorescence analyses suggested that DMPK mutant RNAs recruit and sequester EXP dsRNA-binding proteins. Miller et al. (2000) proposed that the DM1 mutation produces a competing dsRNA-binding substrate that recruits the EXP proteins and sequesters them away from their normal RNA-binding sites during cell differentiation. By radiation hybrid analysis, Ishikawa et al. (1997) mapped the MBNL gene to chromosome 3. Miller et al. (2000) mapped the MBNL gene to 3q25, distal to the DM2 (OMIM Ref. No. 602668) and PROMM (OMIM Ref. No. 600109) loci on 3q21.

Full details of the abovementioned studies are described in the following publications, the disclosure of which are hereby incorporated by reference:
Miller, J. W.; Urbinati, C. R.; Teng-umnuay, P.; Stenberg, M. G.; Byrne, B. J.; Thornton, C. A.; Swanson, M. S.: Recruitment of human muscle blind proteins to (CUG)n expansions associated with myotonic dystrophy. EMBO J. 19: 4439-4448, 2000.; and
Ishikawa, K.; Nagase, T.; Nakajima, D.; Seki, N.; Ohira, M.; Miyajima, N.; Tanaka, A.; Kotani, H.; Nomura, N.; Ohara, O.: Prediction of the coding sequences of unidentified human genes.

Further studies establishing the function and utilities of MBNL are found in John Hopkins OMIM database record ID 606516, and in sited publications numbered 145 and 1973 listed in the bibliography section hereinbelow, which are also hereby incorporated by reference. Phospholamban (PLN, Accession NM_{—}002667) is another VGAM196 host target gene. PLN BINDING SITE is HOST TARGET binding site found in the 3′ untranslated region of mRNA encoded by PLN, corresponding to a HOST TARGET binding site such as BINDING SITE I, BINDING SITE II or BINDING SITE III. Table 2 illustrates the complementarity of the nucleotide sequences of PLN BINDING SITE, designated SEQ ID:946, to the nucleotide sequence of VGAM196 RNA, herein designated VGAM RNA, also designated SEQ ID:531.

Another function of VGAM196 is therefore inhibition of Phospholamban (PLN, Accession NM_{—}002667), a gene which regulates the activity of the calcium pump of cardiac sarcoplasmic reticulum. Accordingly, utilities of VGAM196 include diagnosis, prevention and treatment of diseases and clinical conditions associated with PLN. The function of PLN and its association with various diseases and clinical conditions, has been established by previous studies, as described hereinabove with reference to VGAM104. Solute Carrier Family 12 (sodium/potassium/chloride transporters), Member 2 (SLC12A2, Accession NM_{—}001046) is another VGAM196 host target gene. SLC12A2 BINDING SITE is HOST TARGET binding site found in the 3′ untranslated region of mRNA encoded by SLC12A2, corresponding to a HOST TARGET binding site such as BINDING SITE I, BINDING SITE II or BINDING SITE III. Table 2 illustrates the complementarity of the nucleotide sequences of SLC12A2 BINDING SITE, designated SEQ ID:796, to the nucleotide sequence of VGAM196 RNA, herein designated VGAM RNA, also designated SEQ ID:531.

Another function of VGAM196 is therefore inhibition of Solute Carrier Family 12 (sodium/potassium/chloride transporters), Member 2 (SLC12A2, Accession NM_{—}001046), a gene which transports chloride ions across secretory epithelia. Accordingly, utilities of VGAM196 include diagnosis, prevention and treatment of diseases and clinical conditions associated with SLC12A2. The function of SLC12A2 has been established by previous studies. By moving chloride into epithelial cells, the Na—K—Cl cotransporter aids transcellular movement of chloride across both secretory and absorptive epithelia. Using cDNA probes from the elasmobranch secretory Na—K—Cl cotransporter reported by Xu et al. (1994), Payne et al. (1995) identified the human homolog, which they referred to as NKCC1. By screening cDNA libraries of a human colonic carcinoma cell line, they identified a sequence of 4,115 bases that encoded a deduced protein of 1,212 amino acids with 12 transmembrane segments. By fluorescence in situ hybridization, they localized the cotransporter gene to 5q23.3. Human embryonic kidney cells stably transfected with the full-length cDNA expressed a protein of approximately 170 kD which was recognized by anti-cotransporter antibodies. Following treatment with N-glycosidase F, the molecular mass of the expressed protein was similar to that predicted for the core protein from the cDNA sequence. Evans et al. (2000) tested directly the possibility that the salivary fluid secretory mechanism requires Na+/K+/2Cl− cotransporter-mediated Cl− uptake. They studied the in vivo and in vitro functioning of acinar cells from the parotid glands of mice with targeted disruption of the Nkcc1 gene, which encodes the salivary cotransporter. In wildtype mice Nkcc1 was localized to the basolateral membranes of parotid acinar cells, whereas expression was not detected in duct cells. The lack of functional Nkcc1 resulted in a dramatic reduction (greater than 60%) in the volume of saliva secreted in response to a muscarinic agonist, the primary in situ salivation signal. Expression of the chloride/bicarbonate exchanger AE2 (SLC4A2; 109280) was enhanced, suggesting that this transporter compensates for the loss of functional Nkcc1. The ability of the parotid gland to conserve NaCl was abolished in Nkcc1-deficient mice. Evans et al. (2000) suggested that some cases of ‘idiopathic’ dry mouth disease may have a basis in a defect of Nkcc1.

Full details of the abovementioned studies are described in the following publications, the disclosure of which are hereby incorporated by reference:
Payne, J. A.; Xu, J.-C.; Haas, M.; Lytle, C. Y.; Ward, D.; Forbush, B., III: Primary structure, functional expression, and chromosomal localization of the bumetanide-sensitive Na—K—Cl cotransporter in human colon. J. Biol. Chem. 270: 17977-17985, 1995.; and
Evans, R. L.; Park, K.; Turner, R. J.; Watson, G. E.; Nguyen, H.-V.; Dennett, M. R.; Hand, A. R.; Flagella, M.; Shull, G. E.; Melvin, J. E.: Severe impairment of salivation in Na+/K+/2Cl.

Further studies establishing the function and utilities of SLC12A2 are found in John Hopkins OMIM database record ID 600840, and in sited publications numbered 216 and 2162-2165 listed in the bibliography section hereinbelow, which are also hereby incorporated by reference. Solute Carrier Family 1 (glial high affinity glutamate transporter), Member 3 (SLC1A3, Accession NM_{—}004172) is another VGAM196 host target gene. SLC1A3 BINDING SITE is HOST TARGET binding site found in the 3′ untranslated region of mRNA encoded by SLC1A3, corresponding to a HOST TARGET binding site such as BINDING SITE I, BINDING SITE II or BINDING SITE III. Table 2 illustrates the complementarity of the nucleotide sequences of SLC1A3 BINDING SITE, designated SEQ ID: 1092, to the nucleotide sequence of VGAM196 RNA, herein designated VGAM RNA, also designated SEQ ID:531.

Another function of VGAM196 is therefore inhibition of Solute Carrier Family 1 (glial high affinity glutamate transporter), Member 3 (SLC1A3, Accession NM_{—}004172), a gene which is a transporter molecule that regulates neurotransmitter concentrations at excitatory synapses of the mammalian cns. Accordingly, utilities of VGAM196 include diagnosis, prevention and treatment of diseases and clinical conditions associated with SLC1A3. The function of SLC1A3 has been established by previous studies. Kirschner et al. (1994) mapped the human EAAT1 gene to 5p13 by fluorescence in situ hybridization. They used interspecific backcross analysis to map the murine homolog to chromosome 15 in a region of homology to human 5p13. They commented that the EAAT1 locus may be related to the syndrome of microcephaly and mental retardation observed by Keppen et al. (1992) in association with interstitial deletion of distal band 5p13. In the retina, the glutamate transporter GLAST is expressed in Muller cells, whereas the glutamate transporter GLT1 is found only in cones and various types of bipolar cells. To investigate the functional role of this differential distribution of glutamate transporters, Harada et al. (1998) analyzed Glast and Glt1 mutant mice. In Glast-deficient mice, the electroretinogram b-wave and oscillatory potentials were reduced and retinal damage after ischemia was exacerbated, whereas Glt1-deficient mice showed almost normal electroretinograms and mildly increased retinal damage after ischemia. These results demonstrated that Glast is required for normal signal transmission between photoreceptors and bipolar cells and that both Glast and Glt1 play a neuroprotective role during ischemia in the retina.

Full details of the abovementioned studies are described in the following publications, the disclosure of which are hereby incorporated by reference:
Kirschner, M. A.; Arriza, J. L.; Copeland, N. G.; Gilbert, D. J.; Jenkins, N. A.; Magenis, E.; Amara, S. G.: The mouse and human excitatory amino acid transporter gene (EAAT1) maps to mouse chromosome 15 and a region of syntenic homology on human chromosome 5. Genomics 22: 631-633, 1994.; and
Harada, T.; Harada, C.; Watanabe, M.; Inoue, Y.; Sakagawa, T.; Nakayama, N.; Sasaki, S.; Okuyama, S.; Watase, K.; Wada, K.; Tanaka, K.: Functions of the two glutamate transporters GLAST a.

Further studies establishing the function and utilities of SLC1A3 are found in John Hopkins OMIM database record ID 600111, and in sited publications numbered 676-682 listed in the bibliography section hereinbelow, which are also hereby incorporated by reference. BTB (POZ) Domain Containing 3 (BTBD3, Accession NM_{—}014962) is another VGAM196 host target gene. BTBD3 BINDING SITE is HOST TARGET binding site found in the 3′ untranslated region of mRNA encoded by BTBD3, corresponding to a HOST TARGET binding site such as BINDING SITE I, BINDING SITE II or BINDING SITE III. Table 2 illustrates the complementarity of the nucleotide sequences of BTBD3 BINDING SITE, designated SEQ ID: 1600, to the nucleotide sequence of VGAM196 RNA, herein designated VGAM RNA, also designated SEQ ID:531.

Another function of VGAM196 is therefore inhibition of BTB (POZ) Domain Containing 3 (BTBD3, Accession NM_{—}014962). Accordingly, utilities of VGAM196 include diagnosis, prevention and treatment of diseases and clinical conditions associated with BTBD3. KIAA1237 (Accession XM_{—}087386) is another VGAM196 host target gene. KIAA1237 BINDING SITE is HOST TARGET binding site found in the 3′ untranslated region of mRNA encoded by KIAA1237, corresponding to a HOST TARGET binding site such as BINDING SITE I, BINDING SITE II or BINDING SITE III. Table 2 illustrates the complementarity of the nucleotide sequences of KIAA1237 BINDING SITE, designated SEQ ID:3166, to the nucleotide sequence of VGAM196 RNA, herein designated VGAM RNA, also designated SEQ ID:531.

Another function of VGAM196 is therefore inhibition of KIAA1237 (Accession XM_{—}087386). Accordingly, utilities of VGAM196 include diagnosis, prevention and treatment of diseases and clinical conditions associated with KIAA1237. N-acetylated Alpha-linked Acidic Dipeptidase 2 (NAALAD2, Accession NM_{—}005467) is another VGAM196 host target gene. NAALAD2 BINDING SITE is HOST TARGET binding site found in the 3′ untranslated region of mRNA encoded by NAALAD2, corresponding to a HOST TARGET binding site such as BINDING SITE I, BINDING SITE II or BINDING SITE III. Table 2 illustrates the complementarity of the nucleotide sequences of NAALAD2 BINDING SITE, designated SEQ ID:1215, to the nucleotide sequence of VGAM196 RNA, herein designated VGAM RNA, also designated SEQ ID:531.

Another function of VGAM196 is therefore inhibition of N-acetylated Alpha-linked Acidic Dipeptidase 2 (NAALAD2, Accession NM_{—}005467). Accordingly, utilities of VGAM196 include diagnosis, prevention and treatment of diseases and clinical conditions associated with NAALAD2. Paternally Expressed 10 (PEG10, Accession NM_{—}015068) is another VGAM196 host target gene. PEG10 BINDING SITE is HOST TARGET binding site found in the 3′ untranslated region of mRNA encoded by PEG10, corresponding to a HOST TARGET binding site such as BINDING SITE I, BINDING SITE II or BINDING SITE III. Table 2 illustrates the complementarity of the nucleotide sequences of PEG10 BINDING SITE, designated SEQ ID: 1605, to the nucleotide sequence of VGAM196 RNA, herein designated VGAM RNA, also designated SEQ ID:531.

Another function of VGAM196 is therefore inhibition of Paternally Expressed 10 (PEG10, Accession NM_{—}015068). Accordingly, utilities of VGAM196 include diagnosis, prevention and treatment of diseases and clinical conditions associated with PEG10. FIG. 1 further provides a conceptual description of a novel bioinformatically detected viral gene of the present invention, referred to here as Viral Genomic Address Messenger 197 (VGAM197) viral gene, which modulates expression of respective host target genes thereof, the function and utility of which host target genes is known in the art.

VGAM197 is a novel bioinformatically detected regulatory, non protein coding, viral micro RNA (miRNA) gene. The method by which VGAM197 was detected is described hereinabove with reference to FIGS. 1-8.

VGAM197 gene, herein designated VGAM GENE, is a viral gene contained in the genome of Vaccinia Virus. VGAM197 host target gene, herein designated VGAM HOST TARGET GENE, is a human gene contained in the human genome.

VGAM197 gene encodes a VGAM197 precursor RNA, herein designated VGAM PRECURSOR RNA. Similar to other miRNA genes, and unlike most ordinary genes, VGAM197 precursor RNA does not encode a protein. A nucleotide sequence identical or highly similar to the nucleotide sequence of VGAM197 precursor RNA is designated SEQ ID:183, and is provided hereinbelow with reference to the sequence listing part. Nucleotide sequence SEQ ID:183 is located at position 104417 relative to the genome of Vaccinia Virus.

VGAM197 precursor RNA folds onto itself, forming VGAM197 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM197 folded precursor RNA into VGAM197 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM197 RNA is designated SEQ ID:532, and is provided hereinbelow with reference to the sequence listing part.

VGAM197 host target gene, herein designated VGAM HOST TARGET GENE, encodes a corresponding messenger RNA, VGAM197 host target RNA, herein designated VGAM HOST TARGET RNA. VGAM197 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM197 RNA, herein designated VGAM RNA, binds complementarily to one or more host target binding sites located in untranslated regions of VGAM197 host target RNA, herein designated VGAM HOST TARGET RNA. This complementary binding is due to the fact that the nucleotide sequence of VGAM197 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM197 RNA, herein designated VGAM RNA, may have a different number of host target binding sites in untranslated regions of a VGAM197 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM197 RNA, herein designated VGAM RNA, to host target binding sites on VGAM197 host target RNA, herein designated VGAM HOST TARGET RNA, such as BINDING SITE I, BINDING SITE II and BINDING SITE III, inhibits translation of VGAM197 host target RNA into VGAM197 host target protein, herein designated VGAM HOST TARGET PROTEIN. VGAM host target protein is therefore outlined by a broken line.

It is appreciated that VGAM197 host target gene, herein designated VGAM HOST TARGET GENE, in fact represents a plurality of VGAM197 host target genes. The mRNA of each one of this plurality of VGAM197 host target genes comprises one or more host target binding sites, each having a nucleotide sequence which is at least partly complementary to VGAM197 RNA, herein designated VGAM RNA, and which when bound by VGAM197 RNA causes inhibition of translation of respective one or more VGAM197 host target proteins.

It is further appreciated by one skilled in the art that the mode of translational inhibition illustrated by FIG. 1 with specific reference to translational inhibition exerted by VGAM197 gene, herein designated VGAM GENE, on one or more VGAM197 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM197 is inhibition of expression of host target genes, as part of a novel viral mechanism of attacking a host. Accordingly, utilities of VGAM197 include diagnosis, prevention and treatment of viral infection by Vaccinia Virus. Specific functions, and accordingly utilities, of VGAM197 correlate with, and may be deduced from, the identity of the host target genes which VGAM197 binds and inhibits, and the function of these host target genes, as elaborated hereinbelow.

Nucleotide sequences of the VGAM197 precursor RNA, herein designated VGAM PRECURSOR RNA, and of the ‘diced’ VGAM197 RNA, herein designated VGAM RNA, and a schematic representation of the secondary folding of VGAM197 folded precursor RNA, herein designated VGAM FOLDED PRECURSOR RNA, of VGAM197 are further described hereinbelow with reference to Table 1.

Nucleotide sequences of host target binding sites, such as BINDING SITE-I, BINDING SITE-II and BINDING SITE-III of FIG. 1, found on VGAM197 host target RNA, and schematic representation of the complementarity of each of these host target binding sites to VGAM197 RNA, herein designated VGAM RNA, are described hereinbelow with reference to Table 2.

As mentioned hereinabove with reference to FIG. 1, a function of VGAM197 gene, herein designated VGAM is inhibition of expression of VGAM197 target genes. It is appreciated that specific functions, and accordingly utilities, of VGAM197 correlate with, and may be deduced from, the identity of the target genes which VGAM197 binds and inhibits, and the function of these target genes, as elaborated hereinbelow.

Retinoic Acid Induced 2 (RA12, Accession NM_{—}021785) is a VGAM197 host target gene. RA12 BINDING SITE is HOST TARGET binding site found in the 5′ untranslated region of mRNA encoded by RA12, corresponding to a HOST TARGET binding site such as BINDING SITE I, BINDING SITE II or BINDING SITE III. Table 2 illustrates the complementarity of the nucleotide sequences of RA12 BINDING SITE, designated SEQ ID:1959, to the nucleotide sequence of VGAM197 RNA, herein designated VGAM RNA, also designated SEQ ID:532.

A function of VGAM197 is therefore inhibition of Retinoic Acid Induced 2 (RA12, Accession NM_{—}021785). Accordingly, utilities of VGAM197 include diagnosis, prevention and treatment of diseases and clinical conditions associated with RA12. Uridine Monophosphate Kinase (UMPK, Accession NM_{—}012474) is another VGAM197 host target gene. UMPK BINDING SITE is HOST TARGET binding site found in the 3′ untranslated region of mRNA encoded by UMPK, corresponding to a HOST TARGET binding site such as BINDING SITE I, BINDING SITE II or BINDING SITE III. Table 2 illustrates the complementarity of the nucleotide sequences of UMPK BINDING SITE, designated SEQ ID: 1436, to the nucleotide sequence of VGAM197 RNA, herein designated VGAM RNA, also designated SEQ ID:532.

Another function of VGAM197 is therefore inhibition of Uridine Monophosphate Kinase (UMPK, Accession NM_{—}012474), a gene which catalyzes the phosphorylation of uridine monophosphate to uridine diphosphate. Accordingly, utilities of VGAM197 include diagnosis, prevention and treatment of diseases and clinical conditions associated with UMPK. The function of UMPK has been established by previous studies. This enzyme (EC 2.7.4.4) catalyzes the first step in the production of the pyrimidine nucleoside triphosphates required for RNA and DNA synthesis, namely the phosphorylation of uridine monophosphate to uridine diphosphate. Giblett et al. (1974) found genetic polymorphism of UMPK by means of starch gel electrophoresis. Family studies provided evidence for 3 alleles—UMPK1, UMPK2, and UMPK3—at an autosomal locus. The UMPK1 allele was associated with about 3 times the catalytic activity of the UMPK2 allele, so that UMPK2 homozygotes are relatively deficient of the enzyme. Two of 3 UMPK2 homozygotes were children with prolonged respiratory infection. This suggested to Giblett et al. (1974) that the ability of immunocompetent lymphocytes to respond to appropriate stimuli is impaired in the UMPK2 homozygote in a manner similar to the immune defect resulting from adenosine deaminase deficiency. Alaskan Eskimos have the highest known prevalence of invasive Hemophilus influenzae type B disease, primarily meningitis: in 1 to 5% of all children in the first 2 years of life. In this population a polymorphic variant of UMPK, UMPK-3, is positively associated with invasive HIB disease (relative risk 3.3). No difference in levels of naturally acquired HIB anticapsular antibodies between persons with HIB disease and health controls was found. Thus, the UMPK-3 allele may have a role in mediating nonhumoral immunity to HIB (Petersen et al., 1985). Giblett et al. (1975) showed that UMPK and Rh (OMIM Ref. No. 111700) are linked (Iod score of 2.313 at theta 0.05 on the basis of 4 families). (Rh is located on 1p36.2-p34. Cook and Hamerton (1979) gave 1p32 as the SRO of UMPK.) Satlin et al. (1975) assigned UMPK to chromosome 1 by study of somatic cell hybrids. Using somatic cell hybrids between a mouse cell line deficient in uridine kinase and human cells (Medrano and Green, 1974), Ruddle and Creagan (1975) provisionally assigned UK to chromosome 1. The Goss-Harris method of mapping combined features of recombinational study in families and synteny tests in hybrid cells. As applied to chromosome 1, the method showed that AK2 and UMPK are distal to PGM1 and that the order of the loci is PGM1: UMPK: (AK2, alpha-FUC): ENO1 (Goss and Harris, 1977). Data on gene frequencies of allelic variants were tabulated by Roychoudhury and Nei (1988).

Full details of the abovementioned studies are described in the following publications, the disclosure of which are hereby incorporated by reference:
Giblett, E. R.; Anderson, J. E.; Chen, S.-H.; Teng, Y.-S.; Cohen, F.: Uridine monophosphate kinase: a new genetic polymorphism with possible clinical implications. Am. J. Hum. Genet. 26: 627-635, 1974.; and
Roychoudhury, A. K.; Nei, M.: Human Polymorphic Genes: World Distribution. New York: Oxford Univ. Press (pub.) 1988.

Further studies establishing the function and utilities of UMPK are found in John Hopkins OMIM database record ID 191710, and in sited publications numbered 498, 2989-2992, 190, 2993-2995, 88 and 2996-2997 listed in the bibliography section hereinbelow, which are also hereby incorporated by reference. Zinc Finger Protein 134 (clone pHZ-15) (ZNF134, Accession NM_{—}003435) is another VGAM197 host target gene. ZNF134 BINDING SITE is HOST TARGET binding site found in the 3′ untranslated region of mRNA encoded by ZNF134, corresponding to a HOST TARGET binding site such as BINDING SITE I, BINDING SITE II or BINDING SITE III. Table 2 illustrates the complementarity of the nucleotide sequences of ZNF134 BINDING SITE, designated SEQ ID: 1021, to the nucleotide sequence of VGAM197 RNA, herein designated VGAM RNA, also designated SEQ ID:532.

Another function of VGAM197 is therefore inhibition of Zinc Finger Protein 134 (clone pHZ-15) (ZNF134, Accession NM_{—}003435). Accordingly, utilities of VGAM197 include diagnosis, prevention and treatment of diseases and clinical conditions associated with ZNF134. ARL8 (Accession XM_{—}167671) is another VGAM197 host target gene. ARL8 BINDING SITE is HOST TARGET binding site found in the 3′ untranslated region of mRNA encoded by ARL8, corresponding to a HOST TARGET binding site such as BINDING SITE I, BINDING SITE II or BINDING SITE III. Table 2 illustrates the complementarity of the nucleotide sequences of ARL8 BINDING SITE, designated SEQ ID:3594, to the nucleotide sequence of VGAM197 RNA, herein designated VGAM RNA, also designated SEQ ID:532.

Another function of VGAM197 is therefore inhibition of ARL8 (Accession XM_{—}167671). Accordingly, utilities of VGAM197 include diagnosis, prevention and treatment of diseases and clinical conditions associated with ARL8. KIAA0546 (Accession XM_{—}049055) is another VGAM197 host target gene. KIAA0546 BINDING SITE is HOST TARGET binding site found in the 3′ untranslated region of mRNA encoded by KIAA0546, corresponding to a HOST TARGET binding site such as BINDING SITE I, BINDING SITE II or BINDING SITE III. Table 2 illustrates the complementarity of the nucleotide sequences of KIAA0546 BINDING SITE, designated SEQ ID:2911, to the nucleotide sequence of VGAM197 RNA, herein designated VGAM RNA, also designated SEQ ID:532.

Another function of VGAM197 is therefore inhibition of KIAA0546 (Accession XM_{—}049055). Accordingly, utilities of VGAM197 include diagnosis, prevention and treatment of diseases and clinical conditions associated with KIAA0546. KIAA0644 (Accession NM_{—}014817) is another VGAM197 host target gene. KIAA0644 BINDING SITE is HOST TARGET binding site found in the 3′ untranslated region of mRNA encoded by KIAA0644, corresponding to a HOST TARGET binding site such as BINDING SITE I, BINDING SITE II or BINDING SITE III. Table 2 illustrates the complementarity of the nucleotide sequences of KIAA0644 BINDING SITE, designated SEQ ID:1557, to the nucleotide sequence of VGAM197 RNA, herein designated VGAM RNA, also designated SEQ ID:532.

Another function of VGAM197 is therefore inhibition of KIAA0644 (Accession NM_{—}014817). Accordingly, utilities of VGAM197 include diagnosis, prevention and treatment of diseases and clinical conditions associated with KIAA0644. KIAA1508 (Accession XM_{—}030209) is another VGAM197 host target gene. KIAA1508 BINDING SITE is HOST TARGET binding site found in the 3′ untranslated region of mRNA encoded by KIAA1508, corresponding to a HOST TARGET binding site such as BINDING SITE I, BINDING SITE II or BINDING SITE III. Table 2 illustrates the complementarity of the nucleotide sequences of KIAA1508 BINDING SITE, designated SEQ ID:2614, to the nucleotide sequence of VGAM197 RNA, herein designated VGAM RNA, also designated SEQ ID:532.

Another function of VGAM197 is therefore inhibition of KIAA1508 (Accession XM_{—}030209). Accordingly, utilities of VGAM197 include diagnosis, prevention and treatment of diseases and clinical conditions associated with KIAA1508. 5,10-methenyltetrahydrofolate Synthetase (5-formyltetrahydrofolate cyclo-ligase) (MTHFS, Accession NM_{—}006441) is another VGAM197 host target gene. MTHFS BINDING SITE is HOST TARGET binding site found in the 3′ untranslated region of mRNA encoded by MTHFS, corresponding to a HOST TARGET binding site such as BINDING SITE I, BINDING SITE II or BINDING SITE III. Table 2 illustrates the complementarity of the nucleotide sequences of MTHFS BINDING SITE, designated SEQ ID:1302, to the nucleotide sequence of VGAM197 RNA, herein designated VGAM RNA, also designated SEQ ID:532.

Another function of VGAM197 is therefore inhibition of 5,10-methenyltetrahydrofolate Synthetase (5-formyltetrahydrofolate cyclo-ligase) (MTHFS, Accession NM_{—}006441). Accordingly, utilities of VGAM197 include diagnosis, prevention and treatment of diseases and clinical conditions associated with MTHFS. Succinate-CoA Ligase, ADP-forming, Beta Subunit (SUCLA2, Accession NM_{—}003850) is another VGAM197 host target gene. SUCLA2 BINDING SITE is HOST TARGET binding site found in the 3′ untranslated region of mRNA encoded by SUCLA2, corresponding to a HOST TARGET binding site such as BINDING SITE I, BINDING SITE II or BINDING SITE III. Table 2 illustrates the complementarity of the nucleotide sequences of SUCLA2 BINDING SITE, designated SEQ ID: 1066, to the nucleotide sequence of VGAM197 RNA, herein designated VGAM RNA, also designated SEQ ID:532.

Another function of VGAM197 is therefore inhibition of Succinate-CoA Ligase, ADP-forming, Beta Subunit (SUCLA2, Accession NM_{—}003850). Accordingly, utilities of VGAM197 include diagnosis, prevention and treatment of diseases and clinical conditions associated with SUCLA2. LOC149910 (Accession XM_{—}086699) is another VGAM197 host target gene. LOC149910 BINDING SITE is HOST TARGET binding site found in the 3′ untranslated region of mRNA encoded by LOC149910, corresponding to a HOST TARGET binding site such as BINDING SITE I, BINDING SITE II or BINDING SITE III. Table 2 illustrates the complementarity of the nucleotide sequences of LOC149910 BINDING SITE, designated SEQ ID:3140, to the nucleotide sequence of VGAM197 RNA, herein designated VGAM RNA, also designated SEQ ID:532.

Another function of VGAM197 is therefore inhibition of LOC149910 (Accession XM_{—}086699). Accordingly, utilities of VGAM197 include diagnosis, prevention and treatment of diseases and clinical conditions associated with LOC149910. LOC222171 (Accession XM_{—}166586) is another VGAM197 host target gene. LOC222171 BINDING SITE is HOST TARGET binding site found in the 3′ untranslated region of mRNA encoded by LOC222171, corresponding to a HOST TARGET binding site such as BINDING SITE I, BINDING SITE II or BINDING SITE III. Table 2 illustrates the complementarity of the nucleotide sequences of LOC222171 BINDING SITE, designated SEQ ID:3575, to the nucleotide sequence of VGAM197 RNA, herein designated VGAM RNA, also designated SEQ ID:532.

Another function of VGAM197 is therefore inhibition of LOC222171 (Accession XM_{—}166586). Accordingly, utilities of VGAM197 include diagnosis, prevention and treatment of diseases and clinical conditions associated with LOC222171. FIG. 1 further provides a conceptual description of a novel bioinformatically detected viral gene of the present invention, referred to here as Viral Genomic Address Messenger 198 (VGAM198) viral gene, which modulates expression of respective host target genes thereof, the function and utility of which host target genes is known in the art.

VGAM198 is a novel bioinformatically detected regulatory, non protein coding, viral micro RNA (miRNA) gene. The method by which VGAM198 was detected is described hereinabove with reference to FIGS. 1-8.

VGAM198 gene, herein designated VGAM GENE, is a viral gene contained in the genome of Vaccinia Virus. VGAM198 host target gene, herein designated VGAM HOST TARGET GENE, is a human gene contained in the human genome.

VGAM198 gene encodes a VGAM198 precursor RNA, herein designated VGAM PRECURSOR RNA. Similar to other miRNA genes, and unlike most ordinary genes, VGAM198 precursor RNA does not encode a protein. A nucleotide sequence identical or highly similar to the nucleotide sequence of VGAM198 precursor RNA is designated SEQ ID:184, and is provided hereinbelow with reference to the sequence listing part. Nucleotide sequence SEQ ID:184 is located at position 106759 relative to the genome of Vaccinia Virus.

VGAM198 precursor RNA folds onto itself, forming VGAM198 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM198 folded precursor RNA into VGAM198 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 25%) nucleotide sequence of VGAM198 RNA is designated SEQ ID:533, and is provided hereinbelow with reference to the sequence listing part.

VGAM198 host target gene, herein designated VGAM HOST TARGET GENE, encodes a corresponding messenger RNA, VGAM198 host target RNA, herein designated VGAM HOST TARGET RNA. VGAM198 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM198 RNA, herein designated VGAM RNA, binds complementarily to one or more host target binding sites located in untranslated regions of VGAM198 host target RNA, herein designated VGAM HOST TARGET RNA. This complementary binding is due to the fact that the nucleotide sequence of VGAM198 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM198 RNA, herein designated VGAM RNA, may have a different number of host target binding sites in untranslated regions of a VGAM198 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM198 RNA, herein designated VGAM RNA, to host target binding sites on VGAM198 host target RNA, herein designated VGAM HOST TARGET RNA, such as BINDING SITE I, BINDING SITE II and BINDING SITE III, inhibits translation of VGAM198 host target RNA into VGAM198 host target protein, herein designated VGAM HOST TARGET PROTEIN. VGAM host target protein is therefore outlined by a broken line.

It is appreciated that VGAM198 host target gene, herein designated VGAM HOST TARGET GENE, in fact represents a plurality of VGAM198 host target genes. The mRNA of each one of this plurality of VGAM198 host target genes comprises one or more host target binding sites, each having a nucleotide sequence which is at least partly complementary to VGAM198 RNA, herein designated VGAM RNA, and which when bound by VGAM198 RNA causes inhibition of translation of respective one or more VGAM198 host target proteins.

It is further appreciated by one skilled in the art that the mode of translational inhibition illustrated by FIG. 1 with specific reference to translational inhibition exerted by VGAM198 gene, herein designated VGAM GENE, on one or more VGAM198 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM198 is inhibition of expression of host target genes, as part of a novel viral mechanism of attacking a host. Accordingly, utilities of VGAM198 include diagnosis, prevention and treatment of viral infection by Vaccinia Virus. Specific functions, and accordingly utilities, of VGAM198 correlate with, and may be deduced from, the identity of the host target genes which VGAM198 binds and inhibits, and the function of these host target genes, as elaborated hereinbelow.

Nucleotide sequences of the VGAM198 precursor RNA, herein designated VGAM PRECURSOR RNA, and of the ‘diced’ VGAM198 RNA, herein designated VGAM RNA, and a schematic representation of the secondary folding of VGAM198 folded precursor RNA, herein designated VGAM FOLDED PRECURSOR RNA, of VGAM198 are further described hereinbelow with reference to Table 1.

Nucleotide sequences of host target binding sites, such as BINDING SITE-I, BINDING SITE-II and BINDING SITE-III of FIG. 1, found on VGAM198 host target RNA, and schematic representation of the complementarity of each of these host target binding sites to VGAM198 RNA, herein designated VGAM RNA, are described hereinbelow with reference to Table 2.

As mentioned hereinabove with reference to FIG. 1, a function of VGAM198 gene, herein designated VGAM is inhibition of expression of VGAM198 target genes. It is appreciated that specific functions, and accordingly utilities, of VGAM198 correlate with, and may be deduced from, the identity of the target genes which VGAM198 binds and inhibits, and the function of these target genes, as elaborated hereinbelow.

Zinc Finger Protein 24 (KOX 17) (ZNF24, Accession NM_{—}006965) is a VGAM198 host target gene. ZNF24 BINDING SITE is HOST TARGET binding site found in the 3′ untranslated region of mRNA encoded by ZNF24, corresponding to a HOST TARGET binding site such as BINDING SITE I, BINDING SITE II or BINDING SITE III. Table 2 illustrates the complementarity of the nucleotide sequences of ZNF24 BINDING SITE, designated SEQ ID:1342, to the nucleotide sequence of VGAM198 RNA, herein designated VGAM RNA, also designated SEQ ID:533.

A function of VGAM198 is therefore inhibition of Zinc Finger Protein 24 (KOX 17) (ZNF24, Accession NM_{—}006965). Accordingly, utilities of VGAM198 include diagnosis, prevention and treatment of diseases and clinical conditions associated with ZNF24. Reserved (ALS2CR12, Accession NM_{—}139163) is another VGAM198 host target gene. ALS2CR12 BINDING SITE is HOST TARGET binding site found in the 3′ untranslated region of mRNA encoded by ALS2CR12, corresponding to a HOST TARGET binding site such as BINDING SITE I, BINDING SITE II or BINDING SITE III. Table 2 illustrates the complementarity of the nucleotide sequences of ALS2CR12 BINDING SITE, designated SEQ ID:2471, to the nucleotide sequence of VGAM198 RNA, herein designated VGAM RNA, also designated SEQ ID:533.

Another function of VGAM198 is therefore inhibition of Reserved (ALS2CR12, Accession NM_{—}139163). Accordingly, utilities of VGAM198 include diagnosis, prevention and treatment of diseases and clinical conditions associated with ALS2CR12. FLJ10508 (Accession NM_{—}018118) is another VGAM198 host target gene. FLJ10508 BINDING SITE is HOST TARGET binding site found in the 3′ untranslated region of mRNA encoded by FLJ10508, corresponding to a HOST TARGET binding site such as BINDING SITE I, BINDING SITE II or BINDING SITE III. Table 2 illustrates the complementarity of the nucleotide sequences of FLJ10508 BINDING SITE, designated SEQ ID:1783, to the nucleotide sequence of VGAM198 RNA, herein designated VGAM RNA, also designated SEQ ID:533.

Another function of VGAM198 is therefore inhibition of FLJ10508 (Accession NM_{—}018118). Accordingly, utilities of VGAM198 include diagnosis, prevention and treatment of diseases and clinical conditions associated with FLJ10508. FLJ13197 (Accession NM_{—}024614) is another VGAM198 host target gene. FLJ13197 BINDING SITE is HOST TARGET binding site found in the 3′ untranslated region of mRNA encoded by FLJ13197, corresponding to a HOST TARGET binding site such as BINDING SITE I, BINDING SITE II or BINDING SITE III. Table 2 illustrates the complementarity of the nucleotide sequences of FLJ13197 BINDING SITE, designated SEQ ID:2072, to the nucleotide sequence of VGAM198 RNA, herein designated VGAM RNA, also designated SEQ ID:533.

Another function of VGAM198 is therefore inhibition of FLJ13197 (Accession NM_{—}024614). Accordingly, utilities of VGAM198 include diagnosis, prevention and treatment of diseases and clinical conditions associated with FLJ13197. FLJ21934 (Accession NM_{—}024743) is another VGAM198 host target gene. FLJ21934 BINDING SITE is HOST TARGET binding site found in the 3′ untranslated region of mRNA encoded by FLJ21934, corresponding to a HOST TARGET binding site such as BINDING SITE I, BINDING SITE II or BINDING SITE III. Table 2 illustrates the complementarity of the nucleotide sequences of FLJ21934 BINDING SITE, designated SEQ ID:2085, to the nucleotide sequence of VGAM198 RNA, herein designated VGAM RNA, also designated SEQ ID:533.

Another function of VGAM198 is therefore inhibition of FLJ21934 (Accession NM_{—}024743). Accordingly, utilities of VGAM198 include diagnosis, prevention and treatment of diseases and clinical conditions associated with FLJ21934. FLJ23132 (Accession XM_{—}171194) is another VGAM198 host target gene. FLJ23132 BINDING SITE is HOST TARGET binding site found in the 3′ untranslated region of mRNA encoded by FLJ23132, corresponding to a HOST TARGET binding site such as BINDING SITE I, BINDING SITE II or BINDING SITE III. Table 2 illustrates the complementarity of the nucleotide sequences of FLJ23132 BINDING SITE, designated SEQ ID:3691, to the nucleotide sequence of VGAM198 RNA, herein designated VGAM RNA, also designated SEQ ID:533.

Another function of VGAM198 is therefore inhibition of FLJ23132 (Accession XM_{—}171194). Accordingly, utilities of VGAM198 include diagnosis, prevention and treatment of diseases and clinical conditions associated with FLJ23132. KIAA0470 (Accession NM_{—}014812) is another VGAM198 host target gene. KIAA0470 BINDING SITE is HOST TARGET binding site found in the 3′ untranslated region of mRNA encoded by KIAA0470, corresponding to a HOST TARGET binding site such as BINDING SITE I, BINDING SITE II or BINDING SITE III. Table 2 illustrates the complementarity of the nucleotide sequences of KIAA0470 BINDING SITE, designated SEQ ID:1555, to the nucleotide sequence of VGAM198 RNA, herein designated VGAM RNA, also designated SEQ ID:533.

Another function of VGAM198 is therefore inhibition of KIAA0470 (Accession NM_{—}014812). Accordingly, utilities of VGAM198 include diagnosis, prevention and treatment of diseases and clinical conditions associated with KIAA0470. Leptin Receptor Overlapping Transcript-like 1 (LEPROTL1, Accession NM_{—}015344) is another VGAM198 host target gene. LEPROTL1 BINDING SITE is HOST TARGET binding site found in the 3′ untranslated region of mRNA encoded by LEPROTL1, corresponding to a HOST TARGET binding site such as BINDING SITE I, BINDING SITE II or BINDING SITE III. Table 2 illustrates the complementarity of the nucleotide sequences of LEPROTL1 BINDING SITE, designated SEQ ID: 1620, to the nucleotide sequence of VGAM198 RNA, herein designated VGAM RNA, also designated SEQ ID:533.

Another function of VGAM198 is therefore inhibition of Leptin Receptor Overlapping Transcript-like 1 (LEPROTL1, Accession NM_{—}015344). Accordingly, utilities of VGAM198 include diagnosis, prevention and treatment of diseases and clinical conditions associated with LEPROTL1. Lysosomal Apyrase-like 1 (LYSAL1, Accession NM_{—}004901) is another VGAM198 host target gene. LYSAL1 BINDING SITE is HOST TARGET binding site found in the 5′ untranslated region of mRNA encoded by LYSAL1, corresponding to a HOST TARGET binding site such as BINDING SITE I, BINDING SITE II or BINDING SITE III. Table 2 illustrates the complementarity of the nucleotide sequences of LYSAL1 BINDING SITE, designated SEQ ID:1164, to the nucleotide sequence of VGAM198 RNA, herein designated VGAM RNA, also designated SEQ ID:533.

Another function of VGAM198 is therefore inhibition of Lysosomal Apyrase-like 1 (LYSAL1, Accession NM_{—}004901). Accordingly, utilities of VGAM198 include diagnosis, prevention and treatment of diseases and clinical conditions associated with LYSAL1. RAB40A, Member RAS Oncogene Family (RAB40A, Accession XM_{—}088733) is another VGAM198 host target gene. RAB40A BINDING SITE is HOST TARGET binding site found in the 5′ untranslated region of mRNA encoded by RAB40A, corresponding to a HOST TARGET binding site such as BINDING SITE I, BINDING SITE II or BINDING SITE III. Table 2 illustrates the complementarity of the nucleotide sequences of RAB40A BINDING SITE, designated SEQ ID:3229, to the nucleotide sequence of VGAM198 RNA, herein designated VGAM RNA, also designated SEQ ID:533.

Another function of VGAM198 is therefore inhibition of RAB40A, Member RAS Oncogene Family (RAB40A, Accession XM_{—}088733). Accordingly, utilities of VGAM198 include diagnosis, prevention and treatment of diseases and clinical conditions associated with RAB40A. Serine/threonine Kinase 38 Like (STK38L, Accession XM_{—}044823) is another VGAM198 host target gene. STK38L BINDING SITE is HOST TARGET binding site found in the 3′ untranslated region of mRNA encoded by STK38L, corresponding to a HOST TARGET binding site such as BINDING SITE I, BINDING SITE II or BINDING SITE III. Table 2 illustrates the complementarity of the nucleotide sequences of STK38L BINDING SITE, designated SEQ ID:2845, to the nucleotide sequence of VGAM198 RNA, herein designated VGAM RNA, also designated SEQ ID:533.

Another function of VGAM198 is therefore inhibition of Serine/threonine Kinase 38 Like (STK38L, Accession XM_{—}044823). Accordingly, utilities of VGAM198 include diagnosis, prevention and treatment of diseases and clinical conditions associated with STK38L. Zinc Finger Protein 363 (ZNF363, Accession XM_{—}055989) is another VGAM198 host target gene. ZNF363 BINDING SITE is HOST TARGET binding site found in the 3′ untranslated region of mRNA encoded by ZNF363, corresponding to a HOST TARGET binding site such as BINDING SITE I, BINDING SITE II or BINDING SITE III. Table 2 illustrates the complementarity of the nucleotide sequences of ZNF363 BINDING SITE, designated SEQ ID:2974, to the nucleotide sequence of VGAM198 RNA, herein designated VGAM RNA, also designated SEQ ID:533.

Another function of VGAM198 is therefore inhibition of Zinc Finger Protein 363 (ZNF363, Accession XM_{—}055989). Accordingly, utilities of VGAM198 include diagnosis, prevention and treatment of diseases and clinical conditions associated with ZNF363. LOC146481 (Accession XM_{—}085484) is another VGAM198 host target gene. LOC146481 BINDING SITE is HOST TARGET binding site found in the 3′ untranslated region of mRNA encoded by LOC146481, corresponding to a HOST TARGET binding site such as BINDING SITE I, BINDING SITE II or BINDING SITE III. Table 2 illustrates the complementarity of the nucleotide sequences of LOC146481 BINDING SITE, designated SEQ ID:3093, to the nucleotide sequence of VGAM198 RNA, herein designated VGAM RNA, also designated SEQ ID:533.

Another function of VGAM198 is therefore inhibition of LOC146481 (Accession XM_{—}085484). Accordingly, utilities of VGAM198 include diagnosis, prevention and treatment of diseases and clinical conditions associated with LOC146481. LOC152008 (Accession XM_{—}087363) is another VGAM198 host target gene. LOC152008 BINDING SITE is HOST TARGET binding site found in the 3′ untranslated region of mRNA encoded by LOC152008, corresponding to a HOST TARGET binding site such as BINDING SITE I, BINDING SITE II or BINDING SITE III. Table 2 illustrates the complementarity of the nucleotide sequences of LOC152008 BINDING SITE, designated SEQ ID:3165, to the nucleotide sequence of VGAM198 RNA, herein designated VGAM RNA, also designated SEQ ID:533.

Another function of VGAM198 is therefore inhibition of LOC152008 (Accession XM_{—}087363). Accordingly, utilities of VGAM198 include diagnosis, prevention and treatment of diseases and clinical conditions associated with LOC152008. LOC153020 (Accession XM_{—}087578) is another VGAM198 host target gene. LOC153020 BINDING SITE is HOST TARGET binding site found in the 3′ untranslated region of mRNA encoded by LOC153020, corresponding to a HOST TARGET binding site such as BINDING SITE I, BINDING SITE II or BINDING SITE III. Table 2 illustrates the complementarity of the nucleotide sequences of LOC153020 BINDING SITE, designated SEQ ID:3178, to the nucleotide sequence of VGAM198 RNA, herein designated VGAM RNA, also designated SEQ ID:533.

Another function of VGAM198 is therefore inhibition of LOC153020 (Accession XM_{—}087578). Accordingly, utilities of VGAM198 include diagnosis, prevention and treatment of diseases and clinical conditions associated with LOC153020. LOC199786 (Accession XM_{—}114021) is another VGAM198 host target gene. LOC199786 BINDING SITE is HOST TARGET binding site found in the 3′ untranslated region of mRNA encoded by LOC199786, corresponding to a HOST TARGET binding site such as BINDING SITE I, BINDING SITE II or BINDING SITE III. Table 2 illustrates the complementarity of the nucleotide sequences of LOC199786 BINDING SITE, designated SEQ ID:3433, to the nucleotide sequence of VGAM198 RNA, herein designated VGAM RNA, also designated SEQ ID:533.

Another function of VGAM198 is therefore inhibition of LOC199786 (Accession XM_{—}114021). Accordingly, utilities of VGAM198 include diagnosis, prevention and treatment of diseases and clinical conditions associated with LOC199786. LOC220766 (Accession XM_{—}165471) is another VGAM198 host target gene. LOC220766 BINDING SITE is HOST TARGET binding site found in the 3′ untranslated region of mRNA encoded by LOC220766, corresponding to a HOST TARGET binding site such as BINDING SITE I, BINDING SITE II or BINDING SITE III. Table 2 illustrates the complementarity of the nucleotide sequences of LOC220766 BINDING SITE, designated SEQ ID:3498, to the nucleotide sequence of VGAM198 RNA, herein designated VGAM RNA, also designated SEQ ID:533.

Another function of VGAM198 is therefore inhibition of LOC220766 (Accession XM_{—}165471). Accordingly, utilities of VGAM198 include diagnosis, prevention and treatment of diseases and clinical conditions associated with LOC220766. FIG. 1 further provides a conceptual description of a novel bioinformatically detected viral gene of the present invention, referred to here as Viral Genomic Address Messenger 199 (VGAM199) viral gene, which modulates expression of respective host target genes thereof, the function and utility of which host target genes is known in the art.

VGAM199 is a novel bioinformatically detected regulatory, non protein coding, viral micro RNA (miRNA) gene. The method by which VGAM199 was detected is described hereinabove with reference to FIGS. 1-8.

VGAM199 gene, herein designated VGAM GENE, is a viral gene contained in the genome of Vaccinia Virus. VGAM199 host target gene, herein designated VGAM HOST TARGET GENE, is a human gene contained in the human genome.

VGAM199 gene encodes a VGAM199 precursor RNA, herein designated VGAM PRECURSOR RNA. Similar to other miRNA genes, and unlike most ordinary genes, VGAM199 precursor RNA does not encode a protein. A nucleotide sequence identical or highly similar to the nucleotide sequence of VGAM199 precursor RNA is designated SEQ ID:185, and is provided hereinbelow with reference to the sequence listing part. Nucleotide sequence SEQ ID:185 is located at position 106596 relative to the genome of Vaccinia Virus.

VGAM199 precursor RNA folds onto itself, forming VGAM199 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM199 folded precursor RNA into VGAM199 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM199 RNA is designated SEQ ID:534, and is provided hereinbelow with reference to the sequence listing part.

VGAM199 host target gene, herein designated VGAM HOST TARGET GENE, encodes a corresponding messenger RNA, VGAM199 host target RNA, herein designated VGAM HOST TARGET RNA. VGAM199 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM199 RNA, herein designated VGAM RNA, binds complementarily to one or more host target binding sites located in untranslated regions of VGAM199 host target RNA, herein designated VGAM HOST TARGET RNA. This complementary binding is due to the fact that the nucleotide sequence of VGAM199 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM199 RNA, herein designated VGAM RNA, may have a different number of host target binding sites in untranslated regions of a VGAM199 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM199 RNA, herein designated VGAM RNA, to host target binding sites on VGAM199 host target RNA, herein designated VGAM HOST TARGET RNA, such as BINDING SITE I, BINDING SITE II and BINDING SITE III, inhibits translation of VGAM199 host target RNA into VGAM199 host target protein, herein designated VGAM HOST TARGET PROTEIN. VGAM host target protein is therefore outlined by a broken line.

It is appreciated that VGAM199 host target gene, herein designated VGAM HOST TARGET GENE, in fact represents a plurality of VGAM199 host target genes. The mRNA of each one of this plurality of VGAM199 host target genes comprises one or more host target binding sites, each having a nucleotide sequence which is at least partly complementary to VGAM199 RNA, herein designated VGAM RNA, and which when bound by VGAM199 RNA causes inhibition of translation of respective one or more VGAM199 host target proteins.

It is further appreciated by one skilled in the art that the mode of translational inhibition illustrated by FIG. 1 with specific reference to translational inhibition exerted by VGAM199 gene, herein designated VGAM GENE, on one or more VGAM199 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM199 is inhibition of expression of host target genes, as part of a novel viral mechanism of attacking a host. Accordingly, utilities of VGAM199 include diagnosis, prevention and treatment of viral infection by Vaccinia Virus. Specific functions, and accordingly utilities, of VGAM199 correlate with, and may be deduced from, the identity of the host target genes which VGAM199 binds and inhibits, and the function of these host target genes, as elaborated hereinbelow.

Nucleotide sequences of the VGAM199 precursor RNA, herein designated VGAM PRECURSOR RNA, and of the ‘diced’ VGAM199 RNA, herein designated VGAM RNA, and a schematic representation of the secondary folding of VGAM199 folded precursor RNA, herein designated VGAM FOLDED PRECURSOR RNA, of VGAM199 are further described hereinbelow with reference to Table 1.

Nucleotide sequences of host target binding sites, such as BINDING SITE-I, BINDING SITE-II and BINDING SITE-III of FIG. 1, found on VGAM199 host target RNA, and schematic representation of the complementarity of each of these host target binding sites to VGAM199 RNA, herein designated VGAM RNA, are described hereinbelow with reference to Table 2.

As mentioned hereinabove with reference to FIG. 1, a function of VGAM199 gene, herein designated VGAM is inhibition of expression of VGAM199 target genes. It is appreciated that specific functions, and accordingly utilities, of VGAM199 correlate with, and may be deduced from, the identity of the target genes which VGAM199 binds and inhibits, and the function of these target genes, as elaborated hereinbelow.

A Disintegrin and Metalloproteinase Domain 12 (meltrin alpha) (ADAM12, Accession NM_{—}003474) is a VGAM199 host target gene. ADAM12 BINDING SITE is HOST TARGET binding site found in the 3′ untranslated region of mRNA encoded by ADAM12, corresponding to a HOST TARGET binding site such as BINDING SITE I, BINDING SITE II or BINDING SITE III. Table 2 illustrates the complementarity of the nucleotide sequences of ADAM12 BINDING SITE, designated SEQ ID: 1029, to the nucleotide sequence of VGAM199 RNA, herein designated VGAM RNA, also designated SEQ ID:534.

A function of VGAM199 is therefore inhibition of A Disintegrin and Metalloproteinase Domain 12 (meltrin alpha) (ADAM12, Accession NM_{—}003474), a gene which involved in skeletal muscle regeneration, specifically at the onset of cell fusion. Accordingly, utilities of VGAM199 include diagnosis, prevention and treatment of diseases and clinical conditions associated with ADAM12. The function of ADAM12 has been established by previous studies. To isolate genes related to fertilin expressed in muscle, Yagami-Hiromasa et al. (1995) amplified cDNAs prepared from a mouse myogenic cell line by PCR using degenerative primers for conserved amino acids between fertilin-alpha and -beta (OMIM Ref. No. 601533). They identified 3 novel mouse sequences, which they called meltrins. Similarly to myogenin, a marker of early muscle differentiation, mouse meltrin-alpha is expressed in neonatal muscle and bone, and its expression increases dramatically in response to the induction of differentiation. Immunocytochemical localization and functional expression studies suggested that meltrin-alpha may be involved in myotube formation. Galliano et al. (2000) found by RT-PCR and immunoblot analyses that expression of mouse Adam12 increases during muscle regeneration, while the levels of other ADAMs remain constant. Immunofluorescence analysis revealed staining of small, newly formed muscle fibers in regenerating but not normal adult muscle cells. Using a yeast 2-hybrid screen of a human skeletal muscle cDNA library with the cytoplasmic tail of human ADAM12 as bait, Galliano et al. (2000) determined that the membrane proximal portion of the C-terminal half of myristoylated ADAM12 interacts with muscle-specific alpha-actinin-2 (ACTN2; 102573). Galliano et al. (2000) determined that overexpression of cytosolic ADAM12 containing the ACTN2-binding site inhibits mouse myoblast fusion.

Full details of the abovementioned studies are described in the following publications, the disclosure of which are hereby incorporated by reference:
Galliano, M.-F.; Huet, C.; Frygelius, J.; Polgren, A.; Wewer, U. M.; Engvall, E.: Binding of ADAM12, a marker of skeletal muscle regeneration, to the muscle-specific actin-binding protein, alpha-actinin-2, is required for myoblast fusion. J. Biol. Chem. 275: 13933-13939, 2000.; and
Yagami-Hiromasa, T.; Sato, T.; Kurisaki, T.; Kamijo, K.; Nabeshima, Y.; Fujisawa-Sehara, A.: A metalloprotease-disintegrin participating in myoblast fusion. Nature 377: 652-656, 1995.

Further studies establishing the function and utilities of ADAM12 are found in John Hopkins OMIM database record ID 602714, and in sited publications numbered 263-265 listed in the bibliography section hereinbelow, which are also hereby incorporated by reference. Homeo Box C13 (HOXC13, Accession XM_{—}006804) is another VGAM199 host target gene. HOXC13 BINDING SITE is HOST TARGET binding site found in the 3′ untranslated region of mRNA encoded by HOXC13, corresponding to a HOST TARGET binding site such as BINDING SITE I, BINDING SITE II or BINDING SITE III. Table 2 illustrates the complementarity of the nucleotide sequences of HOXC13 BINDING SITE, designated SEQ ID:2538, to the nucleotide sequence of VGAM199 RNA, herein designated VGAM RNA, also designated SEQ ID:534.

Another function of VGAM199 is therefore inhibition of Homeo Box C13 (HOXC13, Accession XM_{—}006804). Accordingly, utilities of VGAM199 include diagnosis, prevention and treatment of diseases and clinical conditions associated with HOXC13. Nuclear Receptor Interacting Protein 1 (NRIP1, Accession XM_{—}009699) is another VGAM199 host target gene. NRIP1 BINDING SITE is HOST TARGET binding site found in the 3′ untranslated region of mRNA encoded by NRIP1, corresponding to a HOST TARGET binding site such as BINDING SITE I, BINDING SITE II or BINDING SITE III. Table 2 illustrates the complementarity of the nucleotide sequences of NRIP1 BINDING SITE, designated SEQ ID:2549, to the nucleotide sequence of VGAM199 RNA, herein designated VGAM RNA, also designated SEQ ID:534.

Another function of VGAM199 is therefore inhibition of Nuclear Receptor Interacting Protein 1 (NRIP1, Accession XM_{—}009699), a gene which modulates transcriptional activation by the estrogen receptor. Accordingly, utilities of VGAM199 include diagnosis, prevention and treatment of diseases and clinical conditions associated with NRIP1. The function of NRIP1 and its association with various diseases and clinical conditions, has been established by previous studies, as described hereinabove with reference to VGAM119. Ribonucleotide Reductase M2 B (TP53 inducible) (RRM2B, Accession XM_{—}042096) is another VGAM199 host target gene. RRM2B BINDING SITE is HOST TARGET binding site found in the 3′ untranslated region of mRNA encoded by RRM2B, corresponding to a HOST TARGET binding site such as BINDING SITE I, BINDING SITE II or BINDING SITE III. Table 2 illustrates the complementarity of the nucleotide sequences of RRM2B BINDING SITE, designated SEQ ID:2798, to the nucleotide sequence of VGAM199 RNA, herein designated VGAM RNA, also designated SEQ ID:534.

Another function of VGAM199 is therefore inhibition of Ribonucleotide Reductase M2 B (TP53 inducible) (RRM2B, Accession XM_{—}042096). Accordingly, utilities of VGAM199 include diagnosis, prevention and treatment of diseases and clinical conditions associated with RRM2B. Baculoviral IAP Repeat-containing 4 (BIRC4, Accession NM_{—}001167) is another VGAM199 host target gene. BIRC4 BINDING SITE is HOST TARGET binding site found in the 3′ untranslated region of mRNA encoded by BIRC4, corresponding to a HOST TARGET binding site such as BINDING SITE I, BINDING SITE II or BINDING SITE III. Table 2 illustrates the complementarity of the nucleotide sequences of BIRC4 BINDING SITE, designated SEQ ID:806, to the nucleotide sequence of VGAM199 RNA, herein designated VGAM RNA, also designated SEQ ID:534.

Another function of VGAM199 is therefore inhibition of Baculoviral IAP Repeat-containing 4 (BIRC4, Accession NM_{—}001167). Accordingly, utilities of VGAM199 include diagnosis, prevention and treatment of diseases and clinical conditions associated with BIRC4. FLJ11301 (Accession NM_{—}018385) is another VGAM199 host target gene. FLJ11301 BINDING SITE is HOST TARGET binding site found in the 3′ untranslated region of mRNA encoded by FLJ11301, corresponding to a HOST TARGET binding site such as BINDING SITE I, BINDING SITE II or BINDING SITE III. Table 2 illustrates the complementarity of the nucleotide sequences of FLJ11301 BINDING SITE, designated SEQ ID:1822, to the nucleotide sequence of VGAM199 RNA, herein designated VGAM RNA, also designated SEQ ID:534.

Another function of VGAM199 is therefore inhibition of FLJ11301 (Accession NM_{—}018385). Accordingly, utilities of VGAM199 include diagnosis, prevention and treatment of diseases and clinical conditions associated with FLJ11301. LAP1B (Accession XM_{—}035429) is another VGAM199 host target gene. LAP1B BINDING SITE is HOST TARGET binding site found in the 5′ untranslated region of mRNA encoded by LAP1B, corresponding to a HOST TARGET binding site such as BINDING SITE I, BINDING SITE II or BINDING SITE III. Table 2 illustrates the complementarity of the nucleotide sequences of LAP1B BINDING SITE, designated SEQ ID:2696, to the nucleotide sequence of VGAM199 RNA, herein designated VGAM RNA, also designated SEQ ID:534.

Another function of VGAM199 is therefore inhibition of LAP1B (Accession XM_{—}035429). Accordingly, utilities of VGAM199 include diagnosis, prevention and treatment of diseases and clinical conditions associated with LAP1B. MGC11324 (Accession NM_{—}032717) is another VGAM199 host target gene. MGC11324 BINDING SITE is HOST TARGET binding site found in the 3′ untranslated region of mRNA encoded by MGC11324, corresponding to a HOST TARGET binding site such as BINDING SITE I, BINDING SITE II or BINDING SITE III. Table 2 illustrates the complementarity of the nucleotide sequences of MGC11324 BINDING SITE, designated SEQ ID:2273, to the nucleotide sequence of VGAM199 RNA, herein designated VGAM RNA, also designated SEQ ID:534.

Another function of VGAM199 is therefore inhibition of MGC11324 (Accession NM_{—}032717). Accordingly, utilities of VGAM199 include diagnosis, prevention and treatment of diseases and clinical conditions associated with MGC11324. PRO2958 (Accession NM_{—}018546) is another VGAM199 host target gene. PRO2958 BINDING SITE is HOST TARGET binding site found in the 3′ untranslated region of mRNA encoded by PRO2958, corresponding to a HOST TARGET binding site such as BINDING SITE I, BINDING SITE II or BINDING SITE III. Table 2 illustrates the complementarity of the nucleotide sequences of PRO2958 BINDING SITE, designated SEQ ID:1841, to the nucleotide sequence of VGAM199 RNA, herein designated VGAM RNA, also designated SEQ ID:534.

Another function of VGAM199 is therefore inhibition of PRO2958 (Accession NM_{—}018546). Accordingly, utilities of VGAM199 include diagnosis, prevention and treatment of diseases and clinical conditions associated with PRO2958. SH3 Domain Binding Glutamic Acid-rich Protein Like 2 (SH3BGRL2, Accession NM_{—}031469) is another VGAM199 host target gene. SH3BGRL2 BINDING SITE is HOST TARGET binding site found in the 3′ untranslated region of mRNA encoded by SH3BGRL2, corresponding to a HOST TARGET binding site such as BINDING SITE I, BINDING SITE II or BINDING SITE III. Table 2 illustrates the complementarity of the nucleotide sequences of SH3BGRL2 BINDING SITE, designated SEQ ID:2208, to the nucleotide sequence of VGAM199 RNA, herein designated VGAM RNA, also designated SEQ ID:534.

Another function of VGAM199 is therefore inhibition of SH3 Domain Binding Glutamic Acid-rich Protein Like 2 (SH3BGRL2, Accession NM_{—}031469). Accordingly, utilities of VGAM199 include diagnosis, prevention and treatment of diseases and clinical conditions associated with SH3BGRL2. LOC144997 (Accession XM_{—}096702) is another VGAM199 host target gene. LOC144997 BINDING SITE is HOST TARGET binding site found in the 3′ untranslated region of mRNA encoded by LOC144997, corresponding to a HOST TARGET binding site such as BINDING SITE I, BINDING SITE II or BINDING SITE III. Table 2 illustrates the complementarity of the nucleotide sequences of LOC144997 BINDING SITE, designated SEQ ID:3271, to the nucleotide sequence of VGAM199 RNA, herein designated VGAM RNA, also designated SEQ ID:534.

Another function of VGAM199 is therefore inhibition of LOC144997 (Accession XM_{—}096702). Accordingly, utilities of VGAM199 include diagnosis, prevention and treatment of diseases and clinical conditions associated with LOC144997. LOC148809 (Accession XM_{—}086325) is another VGAM199 host target gene. LOC148809 BINDING SITE is HOST TARGET binding site found in the 3′ untranslated region of mRNA encoded by LOC148809, corresponding to a HOST TARGET binding site such as BINDING SITE I, BINDING SITE II or BINDING SITE III. Table 2 illustrates the complementarity of the nucleotide sequences of LOC148809 BINDING SITE, designated SEQ ID:3125, to the nucleotide sequence of VGAM199 RNA, herein designated VGAM RNA, also designated SEQ ID:534.

Another function of VGAM199 is therefore inhibition of LOC148809 (Accession XM_{—}086325). Accordingly, utilities of VGAM199 include diagnosis, prevention and treatment of diseases and clinical conditions associated with LOC148809. LOC219540 (Accession XM_{—}168047) is another VGAM199 host target gene. LOC219540 BINDING SITE is HOST TARGET binding site found in the 3′ untranslated region of mRNA encoded by LOC219540, corresponding to a HOST TARGET binding site such as BINDING SITE I, BINDING SITE II or BINDING SITE III. Table 2 illustrates the complementarity of the nucleotide sequences of LOC219540 BINDING SITE, designated SEQ ID:3612, to the nucleotide sequence of VGAM199 RNA, herein designated VGAM RNA, also designated SEQ ID:534.

Another function of VGAM199 is therefore inhibition of LOC219540 (Accession XM_{—}168047). Accordingly, utilities of VGAM199 include diagnosis, prevention and treatment of diseases and clinical conditions associated with LOC219540. FIG. 1 further provides a conceptual description of a novel bioinformatically detected viral gene of the present invention, referred to here as Viral Genomic Address Messenger 200 (VGAM200) viral gene, which modulates expression of respective host target genes thereof, the function and utility of which host target genes is known in the art.

VGAM200 is a novel bioinformatically detected regulatory, non protein coding, viral micro RNA (miRNA) gene. The method by which VGAM200 was detected is described hereinabove with reference to FIGS. 1-8.

VGAM200 gene, herein designated VGAM GENE, is a viral gene contained in the genome of Vaccinia Virus. VGAM200 host target gene, herein designated VGAM HOST TARGET GENE, is a human gene contained in the human genome.

VGAM200 gene encodes a VGAM200 precursor RNA, herein designated VGAM PRECURSOR RNA. Similar to other miRNA genes, and unlike most ordinary genes, VGAM200 precursor RNA does not encode a protein. A nucleotide sequence identical or highly similar to the nucleotide sequence of VGAM200 precursor RNA is designated SEQ ID:186, and is provided hereinbelow with reference to the sequence listing part. Nucleotide sequence SEQ ID:186 is located at position 106423 relative to the genome of Vaccinia Virus.

VGAM200 precursor RNA folds onto itself, forming VGAM200 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM200 folded precursor RNA into VGAM200 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM200 RNA is designated SEQ ID:535, and is provided hereinbelow with reference to the sequence listing part.

VGAM200 host target gene, herein designated VGAM HOST TARGET GENE, encodes a corresponding messenger RNA, VGAM200 host target RNA, herein designated VGAM HOST TARGET RNA. VGAM200 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM200 RNA, herein designated VGAM RNA, binds complementarily to one or more host target binding sites located in untranslated regions of VGAM200 host target RNA, herein designated VGAM HOST TARGET RNA. This complementary binding is due to the fact that the nucleotide sequence of VGAM200 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM200 RNA, herein designated VGAM RNA, may have a different number of host target binding sites in untranslated regions of a VGAM200 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM200 RNA, herein designated VGAM RNA, to host target binding sites on VGAM200 host target RNA, herein designated VGAM HOST TARGET RNA, such as BINDING SITE I, BINDING SITE II and BINDING SITE III, inhibits translation of VGAM200 host target RNA into VGAM200 host target protein, herein designated VGAM HOST TARGET PROTEIN. VGAM host target protein is therefore outlined by a broken line.

It is appreciated that VGAM200 host target gene, herein designated VGAM HOST TARGET GENE, in fact represents a plurality of VGAM200 host target genes. The mRNA of each one of this plurality of VGAM200 host target genes comprises one or more host target binding sites, each having a nucleotide sequence which is at least partly complementary to VGAM200 RNA, herein designated VGAM RNA, and which when bound by VGAM200 RNA causes inhibition of translation of respective one or more VGAM200 host target proteins.

It is further appreciated by one skilled in the art that the mode of translational inhibition illustrated by FIG. 1 with specific reference to translational inhibition exerted by VGAM200 gene, herein designated VGAM GENE, on one or more VGAM200 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM200 is inhibition of expression of host target genes, as part of a novel viral mechanism of attacking a host. Accordingly, utilities of VGAM200 include diagnosis, prevention and treatment of viral infection by Vaccinia Virus. Specific functions, and accordingly utilities, of VGAM200 correlate with, and may be deduced from, the identity of the host target genes which VGAM200 binds and inhibits, and the function of these host target genes, as elaborated hereinbelow.

Nucleotide sequences of the VGAM200 precursor RNA, herein designated VGAM PRECURSOR RNA, and of the ‘diced’ VGAM200 RNA, herein designated VGAM RNA, and a schematic representation of the secondary folding of VGAM200 folded precursor RNA, herein designated VGAM FOLDED PRECURSOR RNA, of VGAM200 are further described hereinbelow with reference to Table 1.

Nucleotide sequences of host target binding sites, such as BINDING SITE-I, BINDING SITE-II and BINDING SITE-III of FIG. 1, found on VGAM200 host target RNA, and schematic representation of the complementarity of each of these host target binding sites to VGAM200 RNA, herein designated VGAM RNA, are described hereinbelow with reference to Table 2.

As mentioned hereinabove with reference to FIG. 1, a function of VGAM200 gene, herein designated VGAM is inhibition of expression of VGAM200 target genes. It is appreciated that specific functions, and accordingly utilities, of VGAM200 correlate with, and may be deduced from, the identity of the target genes which VGAM200 binds and inhibits, and the function of these target genes, as elaborated hereinbelow.

Prostaglandin E Receptor 3 (subtype EP3) (PTGER3, Accession NM_{—}000957) is a VGAM200 host target gene. PTGER3 BINDING SITE is HOST TARGET binding site found in the 3′ untranslated region of mRNA encoded by PTGER3, corresponding to a HOST TARGET binding site such as BINDING SITE I, BINDING SITE II or BINDING SITE III. Table 2 illustrates the complementarity of the nucleotide sequences of PTGER3 BINDING SITE, designated SEQ ID:790, to the nucleotide sequence of VGAM200 RNA, herein designated VGAM RNA, also designated SEQ ID:535.

A function of VGAM200 is therefore inhibition of Prostaglandin E Receptor 3 (subtype EP3) (PTGER3, Accession NM_{—}000957), a gene which is a G protein-coupled receptor that mediates a variety of physiological and pathophysiological effects. Accordingly, utilities of VGAM200 include diagnosis, prevention and treatment of diseases and clinical conditions associated with PTGER3. The function of PTGER3 has been established by previous studies. Prostaglandin E2 (PGE2) induces uterine contraction by increasing intracellular calcium. To investigate other functions of PGE2 in human uterus, Kotani et al. (2000) isolated 2 prostaglandin E receptor EP3 isoforms by RT-PCR using human uterus poly (A)+ RNA. These EP3 isoforms, named EP3-V and EP3-VI, are composed of 402 and 393 amino acid residues, respectively, which are unique compared with EP3 isoforms of other species. Their N-terminal 359 amino acid residues are identical to those of previously reported human EP3 isoforms, whereas the respective C termini of the 2 isoforms contain a novel amino acid sequence. EP3-V and EP3-VI mRNAs were detected abundantly in human uterus, whereas weak but substantial bands were detected in the lung and kidney in RT-PCR specific for each mRNA. In situ hybridization revealed EP3-V and EP3-VI mRNAs in the human myometrium, but not in the endometrium. The authors concluded that EP3-V and EP3-VI are possibly involved in the proliferation of cells in human myometrium. Animal model experiments lend further support to the function of PTGER3. Fever, a hallmark of disease, is elicited by exogenous pyrogens, i.e., cellular components such as lipopolysaccharide (LPS) of infectious organisms, as well as by noninfectious inflammatory insults. Both stimulate the production of cytokines, such as interleukin-1-beta (ILB; 147720), that act on the brain as endogenous pyrogens. Fever can be suppressed by aspirin-like antiinflammatory drugs. As these drugs share the ability to inhibit prostaglandin biosynthesis, it appeared that a prostaglandin is important in fever generation. Whether prostaglandin E2 (PGE2) is a neural mediator of fever has been debated. PGE2 acts by interacting with 4 subtypes of PGE receptors: EP1, EP2, EP3, and EP4. Ushikubi et al. (1998) generated mice lacking each of these receptors by homologous recombination. Only mice lacking the EP3 receptor failed to show a febrile response to PGE2 and to either IL1B or LPS. The results established that PGE2 mediates fever generation in response to both endogenous and exogenous pyrogens by acting at the EP3 receptor.

It is appreciated that the abovementioned animal model for PTGER3 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ushikubi, F.; Segi, E.; Sugimoto, Y.; Murata, T.; Matsuoka, T.; Kobayashi, T.; Hizaki, H.; Tuboi, K.; Katsuyama, M.; Ichikawa, A.; Tanaka, T.; Yoshida, N.; Narumiya, S.: Impaired febrile response in mice lacking the prostaglandin E receptor subtype EP(3). Nature 395: 281-284, 1998.; and
Kotani, M.; Tanaka, I.; Ogawa, Y.; Suganami, T.; Matsumoto, T.; Muro, S.; Yamamoto, Y.; Sugawara, A.; Yoshimasa, Y.; Sagawa, N.; Narumiya, S.; Nakao, K.: Multiple signal transduction pa.

Further studies establishing the function and utilities of PTGER3 are found in John Hopkins OMIM database record ID 176806, and in sited publications numbered 2493, 2494, 2496, 249 and 2498 listed in the bibliography section hereinbelow, which are also hereby incorporated by reference. Sirtuin Silent Mating Type Information Regulation 2 Homolog 1 (S. cerevisiae) (SIRT1, Accession NM_{—}012238) is another VGAM200 host target gene. SIRT1 BINDING SITE is HOST TARGET binding site found in the 3′ untranslated region of mRNA encoded by SIRT1, corresponding to a HOST TARGET binding site such as BINDING SITE I, BINDING SITE II or BINDING SITE III. Table 2 illustrates the complementarity of the nucleotide sequences of SIRT1 BINDING SITE, designated SEQ ID: 1416, to the nucleotide sequence of VGAM200 RNA, herein designated VGAM RNA, also designated SEQ ID:535.

Another function of VGAM200 is therefore inhibition of Sirtuin Silent Mating Type Information Regulation 2 Homolog 1 (S. cerevisiae) (SIRT1, Accession NM_{—}012238), a gene which may function as intracellular regulatory protein with mono-ADP-ribosyltransferase activity. Accordingly, utilities of VGAM200 include diagnosis, prevention and treatment of diseases and clinical conditions associated with SIRT1. The function of SIRT1 has been established by previous studies. Vaziri et al. (2001) showed that the SIRT1 protein binds and deacetylates the p53 protein (OMIM Ref. No. 191170) with a specificity for its C-terminal lys382 residue, modification of which is implicated in the activation of p53 as a transcription factor. Expression of wildtype SIRT1 in human cells reduced the transcriptional activity of p53. In contrast, expression of a catalytically inactive SIRT1 protein potentiated p53-dependent apoptosis and radiosensitivity. These results suggested that SIRT1 is involved in the regulation of p53 function via deacetylation. Luo et al. (2001) showed that mammalian SIRT1 physically interacts with p53 and attenuates p53-mediated functions. Nicotinamide (vitamin B3) inhibited an NAD-dependent p53 deacetylation induced by SIRT1 and also enhanced the p53 acetylation levels in vivo. Furthermore, SIRT1 repressed p53-dependent apoptosis in response to DNA damage and oxidative stress, whereas expression of a SIRT1 point mutant increased the sensitivity of cells in the stress response.

Full details of the abovementioned studies are described in the following publications, the disclosure of which are hereby incorporated by reference:
Vaziri, H.; Dessain, S. K.; Eaton, E. N.; Imai, S.-I.; Frye, R. A.; Pandita, T. K.; Guarente, L.; Weinberg, R. A.: hSIR2—SIRT1 functions as an NAD-dependent p53 deacetylase. Cell 107: 149-159, 2001.; and
Luo, J.; Nikolaev, A. Y.; Imai, S.; Chen, D.; Su, F.; Shiloh, A.; Guarente, L.; Gu, W.: Negative control of p53 by Sir2-alpha promotes cell survival under stress. Cell 107: 137-148, 2.

Further studies establishing the function and utilities of SIRT1 are found in John Hopkins OMIM database record ID 604479, and in sited publications numbered 1134-113 and 1143-1147 listed in the bibliography section hereinbelow, which are also hereby incorporated by reference. FLJ10898 (Accession XM_{—}002486) is another VGAM200 host target gene. FLJ10898 BINDING SITE is HOST TARGET binding site found in the 3′ untranslated region of mRNA encoded by FLJ10898, corresponding to a HOST TARGET binding site such as BINDING SITE I, BINDING SITE II or BINDING SITE III. Table 2 illustrates the complementarity of the nucleotide sequences of FLJ10898 BINDING SITE, designated SEQ ID:2526, to the nucleotide sequence of VGAM200 RNA, herein designated VGAM RNA, also designated SEQ ID:535.

Another function of VGAM200 is therefore inhibition of FLJ10898 (Accession XM_{—}002486). Accordingly, utilities of VGAM200 include diagnosis, prevention and treatment of diseases and clinical conditions associated with FLJ10898. FIG. 1 further provides a conceptual description of a novel bioinformatically detected viral gene of the present invention, referred to here as Viral Genomic Address Messenger 201 (VGAM201) viral gene, which modulates expression of respective host target genes thereof, the function and utility of which host target genes is known in the art.

VGAM201 is a novel bioinformatically detected regulatory, non protein coding, viral micro RNA (miRNA) gene. The method by which VGAM201 was detected is described hereinabove with reference to FIGS. 1-8.

VGAM201 gene, herein designated VGAM GENE, is a viral gene contained in the genome of Vaccinia Virus. VGAM201 host target gene, herein designated VGAM HOST TARGET GENE, is a human gene contained in the human genome.

VGAM201 gene encodes a VGAM201 precursor RNA, herein designated VGAM PRECURSOR RNA. Similar to other miRNA genes, and unlike most ordinary genes, VGAM201 precursor RNA does not encode a protein. A nucleotide sequence identical or highly similar to the nucleotide sequence of VGAM201 precursor RNA is designated SEQ ID:187, and is provided hereinbelow with reference to the sequence listing part. Nucleotide sequence SEQ ID:187 is located at position 107873 relative to the genome of Vaccinia Virus.

VGAM201 precursor RNA folds onto itself, forming VGAM201 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM201 folded precursor RNA into VGAM201 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM201 RNA is designated SEQ ID:536, and is provided hereinbelow with reference to the sequence listing part.

VGAM201 host target gene, herein designated VGAM HOST TARGET GENE, encodes a corresponding messenger RNA, VGAM201 host target RNA, herein designated VGAM HOST TARGET RNA. VGAM201 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM201 RNA, herein designated VGAM RNA, binds complementarily to one or more host target binding sites located in untranslated regions of VGAM201 host target RNA, herein designated VGAM HOST TARGET RNA. This complementary binding is due to the fact that the nucleotide sequence of VGAM201 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM201 RNA, herein designated VGAM RNA, may have a different number of host target binding sites in untranslated regions of a VGAM201 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM201 RNA, herein designated VGAM RNA, to host target binding sites on VGAM201 host target RNA, herein designated VGAM HOST TARGET RNA, such as BINDING SITE I, BINDING SITE II and BINDING SITE III, inhibits translation of VGAM201 host target RNA into VGAM201 host target protein, herein designated VGAM HOST TARGET PROTEIN. VGAM host target protein is therefore outlined by a broken line.

It is appreciated that VGAM201 host target gene, herein designated VGAM HOST TARGET GENE, in fact represents a plurality of VGAM201 host target genes. The mRNA of each one of this plurality of VGAM201 host target genes comprises one or more host target binding sites, each having a nucleotide sequence which is at least partly complementary to VGAM201 RNA, herein designated VGAM RNA, and which when bound by VGAM201 RNA causes inhibition of translation of respective one or more VGAM201 host target proteins.

It is further appreciated by one skilled in the art that the mode of translational inhibition illustrated by FIG. 1 with specific reference to translational inhibition exerted by VGAM201 gene, herein designated VGAM GENE, on one or more VGAM201 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM201 is inhibition of expression of host target genes, as part of a novel viral mechanism of attacking a host. Accordingly, utilities of VGAM201 include diagnosis, prevention and treatment of viral infection by Vaccinia Virus. Specific functions, and accordingly utilities, of VGAM201 correlate with, and may be deduced from, the identity of the host target genes which VGAM201 binds and inhibits, and the function of these host target genes, as elaborated hereinbelow.

Nucleotide sequences of the VGAM201 precursor RNA, herein designated VGAM PRECURSOR RNA, and of the ‘diced’ VGAM201 RNA, herein designated VGAM RNA, and a schematic representation of the secondary folding of VGAM201 folded precursor RNA, herein designated VGAM FOLDED PRECURSOR RNA, of VGAM201 are further described hereinbelow with reference to Table 1.

Nucleotide sequences of host target binding sites, such as BINDING SITE-I, BINDING SITE-II and BINDING SITE-III of FIG. 1, found on VGAM201 host target RNA, and schematic representation of the complementarity of each of these host target binding sites to VGAM201 RNA, herein designated VGAM RNA, are described hereinbelow with reference to Table 2.

As mentioned hereinabove with reference to FIG. 1, a function of VGAM201 gene, herein designated VGAM is inhibition of expression of VGAM201 target genes. It is appreciated that specific functions, and accordingly utilities, of VGAM201 correlate with, and may be deduced from, the identity of the target genes which VGAM201 binds and inhibits, and the function of these target genes, as elaborated hereinbelow.

FLJ10511 (Accession NM_{—}018120) is a VGAM201 host target gene. FLJ10511 BINDING SITE is HOST TARGET binding site found in the 3′ untranslated region of mRNA encoded by FLJ10511, corresponding to a HOST TARGET binding site such as BINDING SITE I, BINDING SITE II or BINDING SITE III. Table 2 illustrates the complementarity of the nucleotide sequences of FLJ10511 BINDING SITE, designated SEQ ID: 1784, to the nucleotide sequence of VGAM201 RNA, herein designated VGAM RNA, also designated SEQ ID:536.

A function of VGAM201 is therefore inhibition of FLJ10511 (Accession NM_{—}018120). Accordingly, utilities of VGAM201 include diagnosis, prevention and treatment of diseases and clinical conditions associated with FLJ10511. KIAA1462 (Accession XM_{—}166132) is another VGAM201 host target gene. KIAA1462 BINDING SITE is HOST TARGET binding site found in the 3′ untranslated region of mRNA encoded by KIAA1462, corresponding to a HOST TARGET binding site such as BINDING SITE I, BINDING SITE II or BINDING SITE III. Table 2 illustrates the complementarity of the nucleotide sequences of KIAA1462 BINDING SITE, designated SEQ ID:3521, to the nucleotide sequence of VGAM201 RNA, herein designated VGAM RNA, also designated SEQ ID:536.

Another function of VGAM201 is therefore inhibition of KIAA1462 (Accession XM_{—}166132). Accordingly, utilities of VGAM201 include diagnosis, prevention and treatment of diseases and clinical conditions associated with KIAA1462. Mesoderm Development Candidate 2 (MESDC2, Accession XM_{—}051854) is another VGAM201 host target gene. MESDC2 BINDING SITE is HOST TARGET binding site found in the 3′ untranslated region of mRNA encoded by MESDC2, corresponding to a HOST TARGET binding site such as BINDING SITE I, BINDING SITE II or BINDING SITE III. Table 2 illustrates the complementarity of the nucleotide sequences of MESDC2 BINDING SITE, designated SEQ ID:2950, to the nucleotide sequence of VGAM201 RNA, herein designated VGAM RNA, also designated SEQ ID:536.

Another function of VGAM201 is therefore inhibition of Mesoderm Development Candidate 2 (MESDC2, Accession XM_{—}051854). Accordingly, utilities of VGAM201 include diagnosis, prevention and treatment of diseases and clinical conditions associated with MESDC2. LOC149620 (Accession XM_{—}086604) is another VGAM201 host target gene. LOC149620 BINDING SITE is HOST TARGET binding site found in the 5′ untranslated region of mRNA encoded by LOC149620, corresponding to a HOST TARGET binding site such as BINDING SITE I, BINDING SITE II or BINDING SITE III. Table 2 illustrates the complementarity of the nucleotide sequences of LOC149620 BINDING SITE, designated SEQ ID:3135, to the nucleotide sequence of VGAM201 RNA, herein designated VGAM RNA, also designated SEQ ID:536.

Another function of VGAM201 is therefore inhibition of LOC149620 (Accession XM_{—}086604). Accordingly, utilities of VGAM201 include diagnosis, prevention and treatment of diseases and clinical conditions associated with LOC149620. LOC219988 (Accession XM_{—}166223) is another VGAM201 host target gene. LOC219988 BINDING SITE is HOST TARGET binding site found in the 5′ untranslated region of mRNA encoded by LOC219988, corresponding to a HOST TARGET binding site such as BINDING SITE I, BINDING SITE II or BINDING SITE III. Table 2 illustrates the complementarity of the nucleotide sequences of LOC219988 BINDING SITE, designated SEQ ID:3534, to the nucleotide sequence of VGAM201 RNA, herein designated VGAM RNA, also designated SEQ ID:536.

Another function of VGAM201 is therefore inhibition of LOC219988 (Accession XM_{—}166223). Accordingly, utilities of VGAM201 include diagnosis, prevention and treatment of diseases and clinical conditions associated with LOC219988. FIG. 1 further provides a conceptual description of a novel bioinformatically detected viral gene of the present invention, referred to here as Viral Genomic Address Messenger 202 (VGAM202) viral gene, which modulates expression of respective host target genes thereof, the function and utility of which host target genes is known in the art.

VGAM202 is a novel bioinformatically detected regulatory, non protein coding, viral micro RNA (miRNA) gene. The method by which VGAM202 was detected is described hereinabove with reference to FIGS. 1-8.

VGAM202 gene, herein designated VGAM GENE, is a viral gene contained in the genome of Vaccinia Virus. VGAM202 host target gene, herein designated VGAM HOST TARGET GENE, is a human gene contained in the human genome.

VGAM202 gene encodes a VGAM202 precursor RNA, herein designated VGAM PRECURSOR RNA. Similar to other miRNA genes, and unlike most ordinary genes, VGAM202 precursor RNA does not encode a protein. A nucleotide sequence identical or highly similar to the nucleotide sequence of VGAM202 precursor RNA is designated SEQ ID:188, and is provided hereinbelow with reference to the sequence listing part. Nucleotide sequence SEQ ID:188 is located at position 108007 relative to the genome of Vaccinia Virus.

VGAM202 precursor RNA folds onto itself, forming VGAM202 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM202 folded precursor RNA into VGAM202 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM202 RNA is designated SEQ ID:537, and is provided hereinbelow with reference to the sequence listing part.

VGAM202 host target gene, herein designated VGAM HOST TARGET GENE, encodes a corresponding messenger RNA, VGAM202 host target RNA, herein designated VGAM HOST TARGET RNA. VGAM202 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM202 RNA, herein designated VGAM RNA, binds complementarily to one or more host target binding sites located in untranslated regions of VGAM202 host target RNA, herein designated VGAM HOST TARGET RNA. This complementary binding is due to the fact that the nucleotide sequence of VGAM202 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM202 RNA, herein designated VGAM RNA, may have a different number of host target binding sites in untranslated regions of a VGAM202 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM202 RNA, herein designated VGAM RNA, to host target binding sites on VGAM202 host target RNA, herein designated VGAM HOST TARGET RNA, such as BINDING SITE I, BINDING SITE II and BINDING SITE III, inhibits translation of VGAM202 host target RNA into VGAM202 host target protein, herein designated VGAM HOST TARGET PROTEIN. VGAM host target protein is therefore outlined by a broken line.

It is appreciated that VGAM202 host target gene, herein designated VGAM HOST TARGET GENE, in fact represents a plurality of VGAM202 host target genes. The mRNA of each one of this plurality of VGAM202 host target genes comprises one or more host target binding sites, each having a nucleotide sequence which is at least partly complementary to VGAM202 RNA, herein designated VGAM RNA, and which when bound by VGAM202 RNA causes inhibition of translation of respective one or more VGAM202 host target proteins.

It is further appreciated by one skilled in the art that the mode of translational inhibition illustrated by FIG. 1 with specific reference to translational inhibition exerted by VGAM202 gene, herein designated VGAM GENE, on one or more VGAM202 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM202 is inhibition of expression of host target genes, as part of a novel viral mechanism of attacking a host. Accordingly, utilities of VGAM202 include diagnosis, prevention and treatment of viral infection by Vaccinia Virus. Specific functions, and accordingly utilities, of VGAM202 correlate with, and may be deduced from, the identity of the host target genes which VGAM202 binds and inhibits, and the function of these host target genes, as elaborated hereinbelow.

Nucleotide sequences of the VGAM202 precursor RNA, herein designated VGAM PRECURSOR RNA, and of the ‘diced’ VGAM202 RNA, herein designated VGAM RNA, and a schematic representation of the secondary folding of VGAM202 folded precursor RNA, herein designated VGAM FOLDED PRECURSOR RNA, of VGAM202 are further described hereinbelow with reference to Table 1.

Nucleotide sequences of host target binding sites, such as BINDING SITE-I, BINDING SITE-II and BINDING SITE-III of FIG. 1, found on VGAM202 host target RNA, and schematic representation of the complementarity of each of these host target binding sites to VGAM202 RNA, herein designated VGAM RNA, are described hereinbelow with reference to Table 2.

As mentioned hereinabove with reference to FIG. 1, a function of VGAM202 gene, herein designated VGAM is inhibition of expression of VGAM202 target genes. It is appreciated that specific functions, and accordingly utilities, of VGAM202 correlate with, and may be deduced from, the identity of the target genes which VGAM202 binds and inhibits, and the function of these target genes, as elaborated hereinbelow.

Betaine-homocysteine Methyltransferase 2 (BHMT2, Accession NM_{—}017614) is a VGAM202 host target gene. BHMT2 BINDING SITE is HOST TARGET binding site found in the 3′ untranslated region of mRNA encoded by BHMT2, corresponding to a HOST TARGET binding site such as BINDING SITE I, BINDING SITE II or BINDING SITE III. Table 2 illustrates the complementarity of the nucleotide sequences of BHMT2 BINDING SITE, designated SEQ ID:1730, to the nucleotide sequence of VGAM202 RNA, herein designated VGAM RNA, also designated SEQ ID:537.

A function of VGAM202 is therefore inhibition of Betaine-homocysteine Methyltransferase 2 (BHMT2, Accession NM_{—}017614). Accordingly, utilities of VGAM202 include diagnosis, prevention and treatment of diseases and clinical conditions associated with BHMT2. Collagen, Type XV, Alpha 1 (COL15A1, Accession NM_{—}001855) is another VGAM202 host target gene. COL15A1 BINDING SITE is HOST TARGET binding site found in the 3′ untranslated region of mRNA encoded by COL15A1, corresponding to a HOST TARGET binding site such as BINDING SITE I, BINDING SITE II or BINDING SITE III. Table 2 illustrates the complementarity of the nucleotide sequences of COL15A1 BINDING SITE, designated SEQ ID:862, to the nucleotide sequence of VGAM202 RNA, herein designated VGAM RNA, also designated SEQ ID:537.

Another function of VGAM202 is therefore inhibition of Collagen, Type XV, Alpha 1 (COL15A1, Accession NM_{—}001855), a gene which may be involved in maintaining the structure of connective tissue. Accordingly, utilities of VGAM202 include diagnosis, prevention and treatment of diseases and clinical conditions associated with COL15A1. The function of COL15A1 has been established by previous studies. Undulin is a large glycoprotein of the interstitial extracellular matrix. It is restricted to dense and soft connective tissues and is associated with mature collagen fibrils (Schuppan et al., 1990). In SDS-polyacrylamide gels, undulin extracted from tissues has a molecular mass above 1,000 kD; under reducing conditions, it migrates as 270-, 190-, and 180-kD polypeptides By immunoscreening a human placenta cDNA expression library with antibodies against monkey undulin, Just et al. (1991) isolated 2 partial cDNAs, called UN1 and UN2, which encode the C-terminal portions of 2 undulin isoforms. The sequences of UN1 and UN2 are partly identical, and the authors suggested that they represent differentially spliced undulin transcripts. Northern blot analysis of human rhabdomyosarcoma cell poly(A) RNA using a probe specific for UN1 detected approximately 4.2-, 6.5-, and 8.5-kb transcripts; a probe specific for UN2 detected a single, approximately 5-kb transcript. The deduced polypeptides contain a differentially spliced von Willebrand factor (VWF; 193400) A domain and the type III homology domains found in fibronectin (OMIM Ref. No. 135600) and tenascin (OMIM Ref. No. 187380). Whereas UN1 has 7 complete and 1 truncated type III homology domains followed by a short proline-rich C-terminal segment, UN2 has 2 complete and 1 incomplete type III homologies followed by a unique acidic C-terminal domain. The authors stated that the mRNAs related to UN1 likely encode the major chains of the undulin molecule

Full details of the abovementioned studies are described in the following publications, the disclosure of which are hereby incorporated by reference:
Schuppan, D.; Cantaluppi, M. C.; Becker, J.; Veit, A.; Bunte, T.; Troyer, D.; Schuppan, F.; Schmid, M.; Ackermann, R.; Hahn, E. G.: Undulin, an extracellular matrix glycoprotein associated with collagen fibrils. J. Biol. Chem. 265: 8823-8832, 1990.; and
Just, M.; Herbst, H.; Hummel, M.; Durkop, H.; Tripier, D.; Stein, H.; Schuppan, D.: Undulin is a novel member of the fibronectin-tenascin family of extracellular matrix glycoproteins. J.

Further studies establishing the function and utilities of COL15A1 are found in John Hopkins OMIM database record ID 120325, and in sited publications numbered 2793-2804 listed in the bibliography section hereinbelow, which are also hereby incorporated by reference. Platelet-derived Growth Factor Receptor, Alpha Polypeptide (PDGFRA, Accession NM_{—}006206) is another VGAM202 host target gene. PDGFRA BINDING SITE is HOST TARGET binding site found in the 3′ untranslated region of mRNA encoded by PDGFRA, corresponding to a HOST TARGET binding site such as BINDING SITE I, BINDING SITE II or BINDING SITE III. Table 2 illustrates the complementarity of the nucleotide sequences of PDGFRA BINDING SITE, designated SEQ ID:1280, to the nucleotide sequence of VGAM202 RNA, herein designated VGAM RNA, also designated SEQ ID:537.

Another function of VGAM202 is therefore inhibition of Platelet-derived Growth Factor Receptor, Alpha Polypeptide (PDGFRA, Accession NM_{—}006206), a gene which this receptor binds platelet-derived growth factor and has a tyrosine-protein kinase activity. Accordingly, utilities of VGAM202 include diagnosis, prevention and treatment of diseases and clinical conditions associated with PDGFRA. The function of PDGFRA has been established by previous studies. Considerable insight into the role of the sonic hedgehog (OMIM Ref. No. 600725) pathway in vertebrate development and human cancers came from the discovery that mutations in ‘patched’ (PTCH; 601309) are associated with basal cell nevus syndrome (BCNS; 109400), an autosomal dominant disorder combining developmental anomalies and tumors, particularly basal cell carcinomas (BCCs). Sporadic BCCs, the most common human cancer, consistently have abnormalities in the hedgehog pathway, and often mutations in PTCH. In addition, somatic mutations in ‘smoothened’ (SMOH; 601500), another protein in the hedgehog pathway, occur in sporadic BCCs. The downstream molecule GLI1 (OMIM Ref. No. 165220) is known to mediate the biologic effect of the hedgehog pathway and is itself up-regulated in all BCCs. Gli1 can drive the production of BCCs in the mouse when overexpressed in the epidermis. Xie et al. (2001) showed that GLI1 can activate PDGFR-alpha and that functional up-regulation of PDGFR-alpha by GLI1 is accompanied by activation of the Ras-ERK pathway, which is associated with cell proliferation. The relevance of this mechanism in vivo is supported by a high level of expression of PDGFR-alpha in BCCs in mice and humans. From these and other observations, Xie et al. (2001) concluded that increased expression of the PDGFR-alpha gene may be an important mechanism by which mutations in the hedgehog pathway cause BCCs. Animal model experiments lend further support to the function of PDGFRA. Klinghoffer et al. (2001) created 2 complementary lines of knockin mice in which the intracellular signaling domains of one PDGFR had been removed and replaced by those of the other PDGFR. While both lines demonstrated substantial rescue of normal development, substitution of the Pdgfrb signaling domains with those of Pdgfra resulted in varying degrees of vascular disease.

It is appreciated that the abovementioned animal model for PDGFRA is acknowledged by those skilled in the art as a scientifically valid animal model, as can be further appreciated from the publications sited hereinbelow.

Full details of the abovementioned studies are described in the following publications, the disclosure of which are hereby incorporated by reference:
Xie, J.; Aszterbaum, M.; Zhang, X.; Bonifas, J. M.; Zachary, C.; Epstein, E.; McCormick, F.: A role of PDGFR-alpha in basal cell carcinoma proliferation. Proc. Nat. Acad. Sci. 98: 9255-9259, 2001.; and
Klinghoffer, R. A.; Mueting-Nelsen, P. F.; Faerman, A.; Shani, M.; Soriano, P.: The two PDGF receptors maintain conserved signaling in vivo despite divergent embryological functions.

Further studies establishing the function and utilities of PDGFRA are found in John Hopkins OMIM database record ID 173490, and in sited publications numbered 3000-3001, 2452, 3002-3005, 3051, 83 and 833-298 listed in the bibliography section hereinbelow, which are also hereby incorporated by reference. DORFIN (Accession NM_{—}015435) is another VGAM202 host target gene. DORFIN BINDING SITE is HOST TARGET binding site found in the 5′ untranslated region of mRNA encoded by DORFIN, corresponding to a HOST TARGET binding site such as BINDING SITE I, BINDING SITE II or BINDING SITE III. Table 2 illustrates the complementarity of the nucleotide sequences of DORFIN BINDING SITE, designated SEQ ID: 1631, to the nucleotide sequence of VGAM202 RNA, herein designated VGAM RNA, also designated SEQ ID:537.

Another function of VGAM202 is therefore inhibition of DORFIN (Accession NM_{—}015435). Accordingly, utilities of VGAM202 include diagnosis, prevention and treatment of diseases and clinical conditions associated with DORFIN. FLJ20034 (Accession NM_{—}017630) is another VGAM202 host target gene. FLJ20034 BINDING SITE is HOST TARGET binding site found in the 5′ untranslated region of mRNA encoded by FLJ20034, corresponding to a HOST TARGET binding site such as BINDING SITE I, BINDING SITE II or BINDING SITE III. Table 2 illustrates the complementarity of the nucleotide sequences of FLJ20034 BINDING SITE, designated SEQ ID:1732, to the nucleotide sequence of VGAM202 RNA, herein designated VGAM RNA, also designated SEQ ID:537.

Another function of VGAM202 is therefore inhibition of FLJ20034 (Accession NM_{—}017630). Accordingly, utilities of VGAM202 include diagnosis, prevention and treatment of diseases and clinical conditions associated with FLJ20034. KIAA0831 (Accession NM_{—}014924) is another VGAM202 host target gene. KIAA0831 BINDING SITE is HOST TARGET binding site found in the 5′ untranslated region of mRNA encoded by KIAA0831, corresponding to a HOST TARGET binding site such as BINDING SITE I, BINDING SITE II or BINDING SITE III. Table 2 illustrates the complementarity of the nucleotide sequences of KIAA0831 BINDING SITE, designated SEQ ID:1589, to the nucleotide sequence of VGAM202 RNA, herein designated VGAM RNA, also designated SEQ ID:537.

Another function of VGAM202 is therefore inhibition of KIAA0831 (Accession NM_{—}014924). Accordingly, utilities of VGAM202 include diagnosis, prevention and treatment of diseases and clinical conditions associated with KIAA0831. MBLL39 (Accession NM_{—}005757) is another VGAM202 host target gene. MBLL39 BINDING SITE is HOST TARGET binding site found in the 3′ untranslated region of mRNA encoded by MBLL39, corresponding to a HOST TARGET binding site such as BINDING SITE I, BINDING SITE II or BINDING SITE III. Table 2 illustrates the complementarity of the nucleotide sequences of MBLL39 BINDING SITE, designated SEQ ID:1243, to the nucleotide sequence of VGAM202 RNA, herein designated VGAM RNA, also designated SEQ ID:537.

Another function of VGAM202 is therefore inhibition of MBLL39 (Accession NM_{—}005757). Accordingly, utilities of VGAM202 include diagnosis, prevention and treatment of diseases and clinical conditions associated with MBLL39. TSP-NY (Accession NM_{—}032573) is another VGAM202 host target gene. TSP-NY BINDING SITE is HOST TARGET binding site found in the 3′ untranslated region of mRNA encoded by TSP-NY, corresponding to a HOST TARGET binding site such as BINDING SITE I, BINDING SITE II or BINDING SITE III. Table 2 illustrates the complementarity of the nucleotide sequences of TSP-NY BINDING SITE, designated SEQ ID:2261, to the nucleotide sequence of VGAM202 RNA, herein designated VGAM RNA, also designated SEQ ID:537.

Another function of VGAM202 is therefore inhibition of TSP-NY (Accession NM_{—}032573). Accordingly, utilities of VGAM202 include diagnosis, prevention and treatment of diseases and clinical conditions associated with TSP-NY. LOC112840 (Accession NM_{—}080666) is another VGAM202 host target gene. LOC112840 BINDING SITE is HOST TARGET binding site found in the 3′ untranslated region of mRNA encoded by LOC112840, corresponding to a HOST TARGET binding site such as BINDING SITE I, BINDING SITE II or BINDING SITE III. Table 2 illustrates the complementarity of the nucleotide sequences of LOC112840 BINDING SITE, designated SEQ ID:2384, to the nucleotide sequence of VGAM202 RNA, herein designated VGAM RNA, also designated SEQ ID:537.

Another function of VGAM202 is therefore inhibition of LOC112840 (Accession NM_{—}080666). Accordingly, utilities of VGAM202 include diagnosis, prevention and treatment of diseases and clinical conditions associated with LOC112840. LOC136895 (Accession NM_{—}138811) is another VGAM202 host target gene. LOC136895 BINDING SITE is HOST TARGET binding site found in the 3′ untranslated region of mRNA encoded by LOC136895, corresponding to a HOST TARGET binding site such as BINDING SITE I, BINDING SITE II or BINDING SITE III. Table 2 illustrates the complementarity of the nucleotide sequences of LOC136895 BINDING SITE, designated SEQ ID:2458, to the nucleotide sequence of VGAM202 RNA, herein designated VGAM RNA, also designated SEQ ID:537.

Another function of VGAM202 is therefore inhibition of LOC136895 (Accession NM_{—}138811). Accordingly, utilities of VGAM202 include diagnosis, prevention and treatment of diseases and clinical conditions associated with LOC136895. LOC145790 (Accession XM_{—}085234) is another VGAM202 host target gene. LOC145790 BINDING SITE is HOST TARGET binding site found in the 5′ untranslated region of mRNA encoded by LOC145790, corresponding to a HOST TARGET binding site such as BINDING SITE I, BINDING SITE II or BINDING SITE III. Table 2 illustrates the complementarity of the nucleotide sequences of LOC145790 BINDING SITE, designated SEQ ID:3083, to the nucleotide sequence of VGAM202 RNA, herein designated VGAM RNA, also designated SEQ ID:537.

Another function of VGAM202 is therefore inhibition of LOC145790 (Accession XM_{—}085234). Accordingly, utilities of VGAM202 include diagnosis, prevention and treatment of diseases and clinical conditions associated with LOC145790. LOC151040 (Accession XM_{—}087082) is another VGAM202 host target gene. LOC151040 BINDING SITE is HOST TARGET binding site found in the 3′ untranslated region of mRNA encoded by LOC151040, corresponding to a HOST TARGET binding site such as BINDING SITE I, BINDING SITE II or BINDING SITE III. Table 2 illustrates the complementarity of the nucleotide sequences of LOC151040 BINDING SITE, designated SEQ ID:3153, to the nucleotide sequence of VGAM202 RNA, herein designated VGAM RNA, also designated SEQ ID:537.

Another function of VGAM202 is therefore inhibition of LOC151040 (Accession XM_{—}087082). Accordingly, utilities of VGAM202 include diagnosis, prevention and treatment of diseases and clinical conditions associated with LOC151040. LOC157869 (Accession XM_{—}088409) is another VGAM202 host target gene. LOC157869 BINDING SITE is HOST TARGET binding site found in the 3′ untranslated region of mRNA encoded by LOC157869, corresponding to a HOST TARGET binding site such as BINDING SITE I, BINDING SITE II or BINDING SITE III. Table 2 illustrates the complementarity of the nucleotide sequences of LOC157869 BINDING SITE, designated SEQ ID:3207, to the nucleotide sequence of VGAM202 RNA, herein designated VGAM RNA, also designated SEQ ID:537.

Another function of VGAM202 is therefore inhibition of LOC157869 (Accession XM_{—}088409). Accordingly, utilities of VGAM202 include diagnosis, prevention and treatment of diseases and clinical conditions associated with LOC157869. LOC222028 (Accession XM_{—}168382) is another VGAM202 host target gene. LOC222028 BINDING SITE is HOST TARGET binding site found in the 3′ untranslated region of mRNA encoded by LOC222028, corresponding to a HOST TARGET binding site such as BINDING SITE I, BINDING SITE II or BINDING SITE III. Table 2 illustrates the complementarity of the nucleotide sequences of LOC222028 BINDING SITE, designated SEQ ID:3631, to the nucleotide sequence of VGAM202 RNA, herein designated VGAM RNA, also designated SEQ ID:537.

Another function of VGAM202 is therefore inhibition of LOC222028 (Accession XM_{—}168382). Accordingly, utilities of VGAM202 include diagnosis, prevention and treatment of diseases and clinical conditions associated with LOC222028. LOC222252 (Accession XM_{—}168640) is another VGAM202 host target gene. LOC222252 BINDING SITE is HOST TARGET binding site found in the 3′ untranslated region of mRNA encoded by LOC222252, corresponding to a HOST TARGET binding site such as BINDING SITE I, BINDING SITE II or BINDING SITE III. Table 2 illustrates the complementarity of the nucleotide sequences of LOC222252 BINDING SITE, designated SEQ ID:3652, to the nucleotide sequence of VGAM202 RNA, herein designated VGAM RNA, also designated SEQ ID:537.

Another function of VGAM202 is therefore inhibition of LOC222252 (Accession XM_{—}168640). Accordingly, utilities of VGAM202 include diagnosis, prevention and treatment of diseases and clinical conditions associated with LOC222252. FIG. 1 further provides a conceptual description of a novel bioinformatically detected viral gene of the present invention, referred to here as Viral Genomic Address Messenger 203 (VGAM203) viral gene, which modulates expression of respective host target genes thereof, the function and utility of which host target genes is known in the art.

VGAM203 is a novel bioinformatically detected regulatory, non protein coding, viral micro RNA (miRNA) gene. The method by which VGAM203 was detected is described hereinabove with reference to FIGS. 1-8.

VGAM203 gene, herein designated VGAM GENE, is a viral gene contained in the genome of Vaccinia Virus. VGAM203 host target gene, herein designated VGAM HOST TARGET GENE, is a human gene contained in the human genome.

VGAM203 gene encodes a VGAM203 precursor RNA, herein designated VGAM PRECURSOR RNA. Similar to other miRNA genes, and unlike most ordinary genes, VGAM203 precursor RNA does not encode a protein. A nucleotide sequence identical or highly similar to the nucleotide sequence of VGAM203 precursor RNA is designated SEQ ID:189, and is provided hereinbelow with reference to the sequence listing part. Nucleotide sequence SEQ ID:189 is located at position 108588 relative to the genome of Vaccinia Virus.

VGAM203 precursor RNA folds onto itself, forming VGAM203 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM203 folded precursor RNA into VGAM203 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM203 RNA is designated SEQ ID:538, and is provided hereinbelow with reference to the sequence listing part.

VGAM203 host target gene, herein designated VGAM HOST TARGET GENE, encodes a corresponding messenger RNA, VGAM203 host target RNA, herein designated VGAM HOST TARGET RNA. VGAM203 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM203 RNA, herein designated VGAM RNA, binds complementarily to one or more host target binding sites located in untranslated regions of VGAM203 host target RNA, herein designated VGAM HOST TARGET RNA. This complementary binding is due to the fact that the nucleotide sequence of VGAM203 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM203 RNA, herein designated VGAM RNA, may have a different number of host target binding sites in untranslated regions of a VGAM203 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM203 RNA, herein designated VGAM RNA, to host target binding sites on VGAM203 host target RNA, herein designated VGAM HOST TARGET RNA, such as BINDING SITE I, BINDING SITE II and BINDING SITE III, inhibits translation of VGAM203 host target RNA into VGAM203 host target protein, herein designated VGAM HOST TARGET PROTEIN. VGAM host target protein is therefore outlined by a broken line.

It is appreciated that VGAM203 host target gene, herein designated VGAM HOST TARGET GENE, in fact represents a plurality of VGAM203 host target genes. The mRNA of each one of this plurality of VGAM203 host target genes comprises one or more host target binding sites, each having a nucleotide sequence which is at least partly complementary to VGAM203 RNA, herein designated VGAM RNA, and which when bound by VGAM203 RNA causes inhibition of translation of respective one or more VGAM203 host target proteins.

It is further appreciated by one skilled in the art that the mode of translational inhibition illustrated by FIG. 1 with specific reference to translational inhibition exerted by VGAM203 gene, herein designated VGAM GENE, on one or more VGAM203 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM203 is inhibition of expression of host target genes, as part of a novel viral mechanism of attacking a host. Accordingly, utilities of VGAM203 include diagnosis, prevention and treatment of viral infection by Vaccinia Virus. Specific functions, and accordingly utilities, of VGAM203 correlate with, and may be deduced from, the identity of the host target genes which VGAM203 binds and inhibits, and the function of these host target genes, as elaborated hereinbelow.

Nucleotide sequences of the VGAM203 precursor RNA, herein designated VGAM PRECURSOR RNA, and of the ‘diced’ VGAM203 RNA, herein designated VGAM RNA, and a schematic representation of the secondary folding of VGAM203 folded precursor RNA, herein designated VGAM FOLDED PRECURSOR RNA, of VGAM203 are further described hereinbelow with reference to Table 1.

Nucleotide sequences of host target binding sites, such as BINDING SITE-I, BINDING SITE-II and BINDING SITE-III of FIG. 1, found on VGAM203 host target RNA, and schematic representation of the complementarity of each of these host target binding sites to VGAM203 RNA, herein designated VGAM RNA, are described hereinbelow with reference to Table 2.

As mentioned hereinabove with reference to FIG. 1, a function of VGAM203 gene, herein designated VGAM is inhibition of expression of VGAM203 target genes. It is appreciated that specific functions, and accordingly utilities, of VGAM203 correlate with, and may be deduced from, the identity of the target genes which VGAM203 binds and inhibits, and the function of these target genes, as elaborated hereinbelow.

FLJ20485 (Accession NM_{—}019042) is a VGAM203 host target gene. FLJ20485 BINDING SITE is HOST TARGET binding site found in the 3′ untranslated region of mRNA encoded by FLJ20485, corresponding to a HOST TARGET binding site such as BINDING SITE I, BINDING SITE II or BINDING SITE III. Table 2 illustrates the complementarity of the nucleotide sequences of FLJ20485 BINDING SITE, designated SEQ ID: 1875, to the nucleotide sequence of VGAM203 RNA, herein designated VGAM RNA, also designated SEQ ID:538.

A function of VGAM203 is therefore inhibition of FLJ20485 (Accession NM_{—}019042). Accordingly, utilities of VGAM203 include diagnosis, prevention and treatment of diseases and clinical conditions associated with FLJ20485. LOC132617 (Accession XM_{—}067939) is another VGAM203 host target gene. LOC132617 BINDING SITE is HOST TARGET binding site found in the 3′ untranslated region of mRNA encoded by LOC132617, corresponding to a HOST TARGET binding site such as BINDING SITE I, BINDING SITE II or BINDING SITE III. Table 2 illustrates the complementarity of the nucleotide sequences of LOC132617 BINDING SITE, designated SEQ ID:3040, to the nucleotide sequence of VGAM203 RNA, herein designated VGAM RNA, also designated SEQ ID:538.

Another function of VGAM203 is therefore inhibition of LOC132617 (Accession XM_{—}067939). Accordingly, utilities of VGAM203 include diagnosis, prevention and treatment of diseases and clinical conditions associated with LOC132617. FIG. 1 further provides a conceptual description of a novel bioinformatically detected viral gene of the present invention, referred to here as Viral Genomic Address Messenger 204 (VGAM204) viral gene, which modulates expression of respective host target genes thereof, the function and utility of which host target genes is known in the art.

VGAM204 is a novel bioinformatically detected regulatory, non protein coding, viral micro RNA (miRNA) gene. The method by which VGAM204 was detected is described hereinabove with reference to FIGS. 1-8.

VGAM204 gene, herein designated VGAM GENE, is a viral gene contained in the genome of Vaccinia Virus. VGAM204 host target gene, herein designated VGAM HOST TARGET GENE, is a human gene contained in the human genome.

VGAM204 gene encodes a VGAM204 precursor RNA, herein designated VGAM PRECURSOR RNA. Similar to other miRNA genes, and unlike most ordinary genes, VGAM204 precursor RNA does not encode a protein. A nucleotide sequence identical or highly similar to the nucleotide sequence of VGAM204 precursor RNA is designated SEQ ID:190, and is provided hereinbelow with reference to the sequence listing part. Nucleotide sequence SEQ ID:190 is located at position 108944 relative to the genome of Vaccinia Virus.

VGAM204 precursor RNA folds onto itself, forming VGAM204 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM204 folded precursor RNA into VGAM204 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM204 RNA is designated SEQ ID:539, and is provided hereinbelow with reference to the sequence listing part.

VGAM204 host target gene, herein designated VGAM HOST TARGET GENE, encodes a corresponding messenger RNA, VGAM204 host target RNA, herein designated VGAM HOST TARGET RNA. VGAM204 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM204 RNA, herein designated VGAM RNA, binds complementarily to one or more host target binding sites located in untranslated regions of VGAM204 host target RNA, herein designated VGAM HOST TARGET RNA. This complementary binding is due to the fact that the nucleotide sequence of VGAM204 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM204 RNA, herein designated VGAM RNA, may have a different number of host target binding sites in untranslated regions of a VGAM204 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM204 RNA, herein designated VGAM RNA, to host target binding sites on VGAM204 host target RNA, herein designated VGAM HOST TARGET RNA, such as BINDING SITE I, BINDING SITE II and BINDING SITE III, inhibits translation of VGAM204 host target RNA into VGAM204 host target protein, herein designated VGAM HOST TARGET PROTEIN. VGAM host target protein is therefore outlined by a broken line.

It is appreciated that VGAM204 host target gene, herein designated VGAM HOST TARGET GENE, in fact represents a plurality of VGAM204 host target genes. The mRNA of each one of this plurality of VGAM204 host target genes comprises one or more host target binding sites, each having a nucleotide sequence which is at least partly complementary to VGAM204 RNA, herein designated VGAM RNA, and which when bound by VGAM204 RNA causes inhibition of translation of respective one or more VGAM204 host target proteins.

It is further appreciated by one skilled in the art that the mode of translational inhibition illustrated by FIG. 1 with specific reference to translational inhibition exerted by VGAM204 gene, herein designated VGAM GENE, on one or more VGAM204 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM204 is inhibition of expression of host target genes, as part of a novel viral mechanism of attacking a host. Accordingly, utilities of VGAM204 include diagnosis, prevention and treatment of viral infection by Vaccinia Virus. Specific functions, and accordingly utilities, of VGAM204 correlate with, and may be deduced from, the identity of the host target genes which VGAM204 binds and inhibits, and the function of these host target genes, as elaborated hereinbelow.

Nucleotide sequences of the VGAM204 precursor RNA, herein designated VGAM PRECURSOR RNA, and of the ‘diced’ VGAM204 RNA, herein designated VGAM RNA, and a schematic representation of the secondary folding of VGAM204 folded precursor RNA, herein designated VGAM FOLDED PRECURSOR RNA, of VGAM204 are further described hereinbelow with reference to Table 1.

Nucleotide sequences of host target binding sites, such as BINDING SITE-I, BINDING SITE-II and BINDING SITE-III of FIG. 1, found on VGAM204 host target RNA, and schematic representation of the complementarity of each of these host target binding sites to VGAM204 RNA, herein designated VGAM RNA, are described hereinbelow with reference to Table 2.

As mentioned hereinabove with reference to FIG. 1, a function of VGAM204 gene, herein designated VGAM is inhibition of expression of VGAM204 target genes. It is appreciated that specific functions, and accordingly utilities, of VGAM204 correlate with, and may be deduced from, the identity of the target genes which VGAM204 binds and inhibits, and the function of these target genes, as elaborated hereinbelow.

Period Homolog 2 (Drosophila) (PER2, Accession NM_{—}022817) is a VGAM204 host target gene. PER2 BINDING SITE is HOST TARGET binding site found in the 3′ untranslated region of mRNA encoded by PER2, corresponding to a HOST TARGET binding site such as BINDING SITE I, BINDING SITE II or BINDING SITE III. Table 2 illustrates the complementarity of the nucleotide sequences of PER2 BINDING SITE, designated SEQ ID:2013, to the nucleotide sequence of VGAM204 RNA, herein designated VGAM RNA, also designated SEQ ID:539.

A function of VGAM204 is therefore inhibition of Period Homolog 2 (Drosophila) (PER2, Accession NM_{—}022817), a gene which Period homolog 2; putative circadian clock protein; has a PAS dimerization domain. Accordingly, utilities of VGAM204 include diagnosis, prevention and treatment of diseases and clinical conditions associated with PER2. The function of PER2 has been established by previous studies. To investigate the biologic role of NPAS2 (OMIM Ref. No. 603347), Reick et al. (2001) prepared a neuroblastoma cell line capable of conditional induction of the NPAS2:BMAL1 (OMIM Ref. No. 602550) heterodimer and identified putative target genes by representational difference analysis, DNA microarrays, and Northern blotting. Coinduction of NPAS2 and BMAL1 activated transcription of the endogenous Per1, Per2, and Cry1 (OMIM Ref. No. 601933) genes, which encode negatively activating components of the circadian regulatory apparatus, and repressed transcription of the endogenous BMAL1 gene. Analysis of the frontal cortex of wildtype mice kept in a 24-hour light-dark cycle revealed that Per1, Per2, and Cry1 mRNA levels were elevated during darkness and reduced during light, whereas BMAL1 mRNA displayed the opposite pattern. In situ hybridization assays of mice kept in constant darkness revealed that Per2 mRNA abundance did not oscillate as a function of circadian cycle in NPAS2-deficient mice. Thus, NPAS2 likely functions as part of a molecular clock operative in the mammalian forebrain. Animal model experiments lend further support to the function of PER2. Shearman et al. (2000) demonstrated that in the mouse, the core mechanism for the master circadian clock consists of interacting positive and negative transcription and translation feedback loops. Analysis of Clock/Clock (OMIM Ref. No. 601851) mutant mice, homozygous Per2 mutants, and Cry-deficient mice revealed substantially altered Bmal1 (OMIM Ref. No. 602550) rhythms, consistent with a dominant role of Per2 in the positive regulation of the Bmal1 loop. In vitro analysis of Cry inhibition of Clock: Bmal1-mediated transcription shows that the inhibition is through direct protein-protein interactions, independent of the Per and Tim (OMIM Ref. No. 603887) proteins. Per2 is a positive regulator of the Bmal1 loop, and Cry1 and Cry2 are the negative regulators of the Period and Cryptochrome cycles.

It is appreciated that the abovementioned animal model for PER2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Shearman, L. P.; Sriram, S.; Weaver, D. R.; Maywood, E. S.; Chaves, I.; Zheng, B.; Kume, K.; Lee, C. C.; van der Horst, G. T. J.; Hastings, M. H.; Reppert, S. M.: Interacting molecular loops in the mammalian circadian clock. Science 288: 1013-1019, 2000.; and
Shearman, L. P.; Zylka, M. J.; Weaver, D. R.; Kolakowski, L. F., Jr.; Reppert, S. M.: Two period homologs: circadian expression and photic regulation in the suprachiasmatic nuclei. Neu.

Further studies establishing the function and utilities of PER2 are found in John Hopkins OMIM database record ID 603426, and in sited publications numbered 213-214, 1451-1452, 312, 1865-186 and 1962 listed in the bibliography section hereinbelow, which are also hereby incorporated by reference. Solute Carrier Family 14 (urea transporter), Member 2 (SLC14A2, Accession NM_{—}007163) is another VGAM204 host target gene. SLC14A2 BINDING SITE is HOST TARGET binding site found in the 3′ untranslated region of mRNA encoded by SLC14A2, corresponding to a HOST TARGET binding site such as BINDING SITE I, BINDING SITE II or BINDING SITE III. Table 2 illustrates the complementarity of the nucleotide sequences of SLC14A2 BINDING SITE, designated SEQ ID:1360, to the nucleotide sequence of VGAM204 RNA, herein designated VGAM RNA, also designated SEQ ID:539.

Another function of VGAM204 is therefore inhibition of Solute Carrier Family 14 (urea transporter), Member 2 (SLC14A2, Accession NM_{—}007163), a gene which is a renal urea transporter 2. Accordingly, utilities of VGAM204 include diagnosis, prevention and treatment of diseases and clinical conditions associated with SLC14A2. The function of SLC14A2 has been established by previous studies. Genetic variation in proteins that determine sodium reabsorption and excretion significantly influences blood pressure. Ranade et al. (2001) investigated whether nucleotide variation in human UT2 could be associated with variation in blood pressure. Seven single-nucleotide polymorphisms (SNPs) were identified, including val227 to ile and ala357 to thr. Over 1,000 hypertensive and low-normotensive individuals of Chinese origin were genotyped. The ile227 and ala357 alleles were associated with low diastolic blood pressure in men but not women, with odds ratios 2.1 (95% confidence interval 1.5-2.7, P less than 0.001) and 1.5 (95% confidence interval 1.2-1.8, P less than 0.001), respectively. There was a similar trend for systolic blood pressure, and odds ratios for the ile227 and ala357 alleles were 1.7 (95% confidence interval 1.2-2.3, P=0.002) and 1.3 (95% confidence interval 1.1-1.6, P=0.007), respectively, in men. DeStefano et al. (1998) identified a locus for orthostatic hypotension (OHDS; 143850) on chromosome 18q, with a peak Iod score of 3.92 at D18S1367 in 2 linked families. The proximity of human UT2 makes it a potential candidate gene for this autosomal dominant disorder.

Full details of the abovementioned studies are described in the following publications, the disclosure of which are hereby incorporated by reference:
Ranade, K.; Wu, K.-W.; Hwu, C.-M.; Ting, C.-T.; Pei, D.; Pesich, R.; Hebert, J.; Chen, Y.-D. I.; Pratt, R.; Olshen, R.; Masaki, K.; Risch, N.; Cox, D. R.; Botstein, D.: Genetic variation in the human urea transporter-2 is associated with variation in blood pressure. Hum. Molec. Genet. 10: 2157-2164, 2001.; and
DeStefano, A. L.; Baldwin, C. T.; Burzstyn, M.; Gavras, I.; Handy, D. E.; Joost, O.; Martel, T.; Nicolaou, M.; Schwartz, F.; Streeten, D. H. P.; Farrer, L. A.; Gavras, H.: Autosomal domin.

Further studies establishing the function and utilities of SLC14A2 are found in John Hopkins OMIM database record ID 601611, and in sited publications numbered 2819-2052 listed in the bibliography section hereinbelow, which are also hereby incorporated by reference. Chromosome 20 Open Reading Frame 82 (C20orf82, Accession XM_{—}097736) is another VGAM204 host target gene. C20orf82 BINDING SITE is HOST TARGET binding site found in the 3′ untranslated region of mRNA encoded by C20orf82, corresponding to a HOST TARGET binding site such as BINDING SITE I, BINDING SITE II or BINDING SITE III. Table 2 illustrates the complementarity of the nucleotide sequences of C20orf82 BINDING SITE, designated SEQ ID:3316, to the nucleotide sequence of VGAM204 RNA, herein designated VGAM RNA, also designated SEQ ID:539.

Another function of VGAM204 is therefore inhibition of Chromosome 20 Open Reading Frame 82 (C20orf82, Accession XM_{—}097736). Accordingly, utilities of VGAM204 include diagnosis, prevention and treatment of diseases and clinical conditions associated with C20orf82. DKFZp566D234 (Accession XM_{—}030162) is another VGAM204 host target gene. DKFZp566D234 BINDING SITE is HOST TARGET binding site found in the 3′ untranslated region of mRNA encoded by DKFZp566D234, corresponding to a HOST TARGET binding site such as BINDING SITE I, BINDING SITE II or BINDING SITE III. Table 2 illustrates the complementarity of the nucleotide sequences of DKFZp566D234 BINDING SITE, designated SEQ ID:2610, to the nucleotide sequence of VGAM204 RNA, herein designated VGAM RNA, also designated SEQ ID:539.

Another function of VGAM204 is therefore inhibition of DKFZp566D234 (Accession XM_{—}030162). Accordingly, utilities of VGAM204 include diagnosis, prevention and treatment of diseases and clinical conditions associated with DKFZp566D234. EFS2 (Accession NM_{—}005864) is another VGAM204 host target gene. EFS2 BINDING SITE is HOST TARGET binding site found in the 3′ untranslated region of mRNA encoded by EFS2, corresponding to a HOST TARGET binding site such as BINDING SITE I, BINDING SITE II or BINDING SITE III. Table 2 illustrates the complementarity of the nucleotide sequences of EFS2 BINDING SITE, designated SEQ ID:1257, to the nucleotide sequence of VGAM204 RNA, herein designated VGAM RNA, also designated SEQ ID:539.

Another function of VGAM204 is therefore inhibition of EFS2 (Accession NM_{—}005864). Accordingly, utilities of VGAM204 include diagnosis, prevention and treatment of diseases and clinical conditions associated with EFS2. FLJ13194 (Accession NM_{—}025146) is another VGAM204 host target gene. FLJ13194 BINDING SITE is HOST TARGET binding site found in the 3′ untranslated region of mRNA encoded by FLJ13194, corresponding to a HOST TARGET binding site such as BINDING SITE I, BINDING SITE II or BINDING SITE III. Table 2 illustrates the complementarity of the nucleotide sequences of FLJ13194 BINDING SITE, designated SEQ ID:2140, to the nucleotide sequence of VGAM204 RNA, herein designated VGAM RNA, also designated SEQ ID:539.

Another function of VGAM204 is therefore inhibition of FLJ13194 (Accession NM_{—}025146). Accordingly, utilities of VGAM204 include diagnosis, prevention and treatment of diseases and clinical conditions associated with FLJ13194. Zinc Finger Protein 291 (ZNF291, Accession NM_{—}020843) is another VGAM204 host target gene. ZNF291 BINDING SITE is HOST TARGET binding site found in the 3′ untranslated region of mRNA encoded by ZNF291, corresponding to a HOST TARGET binding site such as BINDING SITE I, BINDING SITE II or BINDING SITE III. Table 2 illustrates the complementarity of the nucleotide sequences of ZNF291 BINDING SITE, designated SEQ ID: 1930, to the nucleotide sequence of VGAM204 RNA, herein designated VGAM RNA, also designated SEQ ID:539.

Another function of VGAM204 is therefore inhibition of Zinc Finger Protein 291 (ZNF291, Accession NM_{—}020843). Accordingly, utilities of VGAM204 include diagnosis, prevention and treatment of diseases and clinical conditions associated with ZNF291. LOC157503 (Accession XM_{—}098767) is another VGAM204 host target gene. LOC157503 BINDING SITE is HOST TARGET binding site found in the 3′ untranslated region of mRNA encoded by LOC157503, corresponding to a HOST TARGET binding site such as BINDING SITE I, BINDING SITE II or BINDING SITE III. Table 2 illustrates the complementarity of the nucleotide sequences of LOC157503 BINDING SITE, designated SEQ ID:3380, to the nucleotide sequence of VGAM204 RNA, herein designated VGAM RNA, also designated SEQ ID:539.

Another function of VGAM204 is therefore inhibition of LOC157503 (Accession XM_{—}098767). Accordingly, utilities of VGAM204 include diagnosis, prevention and treatment of diseases and clinical conditions associated with LOC157503. LOC254431 (Accession XM_{—}173024) is another VGAM204 host target gene. LOC254431 BINDING SITE is HOST TARGET binding site found in the 3′ untranslated region of mRNA encoded by LOC254431, corresponding to a HOST TARGET binding site such as BINDING SITE I, BINDING SITE II or BINDING SITE III. Table 2 illustrates the complementarity of the nucleotide sequences of LOC254431 BINDING SITE, designated SEQ ID:3716, to the nucleotide sequence of VGAM204 RNA, herein designated VGAM RNA, also designated SEQ ID:539.

Another function of VGAM204 is therefore inhibition of LOC254431 (Accession XM_{—}173024). Accordingly, utilities of VGAM204 include diagnosis, prevention and treatment of diseases and clinical conditions associated with LOC254431. FIG. 1 further provides a conceptual description of a novel bioinformatically detected viral gene of the present invention, referred to here as Viral Genomic Address Messenger 205 (VGAM205) viral gene, which modulates expression of respective host target genes thereof, the function and utility of which host target genes is known in the art.

VGAM205 is a novel bioinformatically detected regulatory, non protein coding, viral micro RNA (miRNA) gene. The method by which VGAM205 was detected is described hereinabove with reference to FIGS. 1-8.

VGAM205 gene, herein designated VGAM GENE, is a viral gene contained in the genome of Vaccinia Virus. VGAM205 host target gene, herein designated VGAM HOST TARGET GENE, is a human gene contained in the human genome.

VGAM205 gene encodes a VGAM205 precursor RNA, herein designated VGAM PRECURSOR RNA. Similar to other miRNA genes, and unlike most ordinary genes, VGAM205 precursor RNA does not encode a protein. A nucleotide sequence identical or highly similar to the nucleotide sequence of VGAM205 precursor RNA is designated SEQ ID:191, and is provided hereinbelow with reference to the sequence listing part. Nucleotide sequence SEQ ID:191 is located at position 108758 relative to the genome of Vaccinia Virus.

VGAM205 precursor RNA folds onto itself, forming VGAM205 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM205 folded precursor RNA into VGAM205 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM205 RNA is designated SEQ ID:540, and is provided hereinbelow with reference to the sequence listing part.

VGAM205 host target gene, herein designated VGAM HOST TARGET GENE, encodes a corresponding messenger RNA, VGAM205 host target RNA, herein designated VGAM HOST TARGET RNA. VGAM205 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM205 RNA, herein designated VGAM RNA, binds complementarily to one or more host target binding sites located in untranslated regions of VGAM205 host target RNA, herein designated VGAM HOST TARGET RNA. This complementary binding is due to the fact that the nucleotide sequence of VGAM205 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM205 RNA, herein designated VGAM RNA, may have a different number of host target binding sites in untranslated regions of a VGAM205 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM205 RNA, herein designated VGAM RNA, to host target binding sites on VGAM205 host target RNA, herein designated VGAM HOST TARGET RNA, such as BINDING SITE I, BINDING SITE II and BINDING SITE III, inhibits translation of VGAM205 host target RNA into VGAM205 host target protein, herein designated VGAM HOST TARGET PROTEIN. VGAM host target protein is therefore outlined by a broken line.

It is appreciated that VGAM205 host target gene, herein designated VGAM HOST TARGET GENE, in fact represents a plurality of VGAM205 host target genes. The mRNA of each one of this plurality of VGAM205 host target genes comprises one or more host target binding sites, each having a nucleotide sequence which is at least partly complementary to VGAM205 RNA, herein designated VGAM RNA, and which when bound by VGAM205 RNA causes inhibition of translation of respective one or more VGAM205 host target proteins.

It is further appreciated by one skilled in the art that the mode of translational inhibition illustrated by FIG. 1 with specific reference to translational inhibition exerted by VGAM205 gene, herein designated VGAM GENE, on one or more VGAM205 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM205 is inhibition of expression of host target genes, as part of a novel viral mechanism of attacking a host. Accordingly, utilities of VGAM205 include diagnosis, prevention and treatment of viral infection by Vaccinia Virus. Specific functions, and accordingly utilities, of VGAM205 correlate with, and may be deduced from, the identity of the host target genes which VGAM205 binds and inhibits, and the function of these host target genes, as elaborated hereinbelow.

Nucleotide sequences of the VGAM205 precursor RNA, herein designated VGAM PRECURSOR RNA, and of the ‘diced’ VGAM205 RNA, herein designated VGAM RNA, and a schematic representation of the secondary folding of VGAM205 folded precursor RNA, herein designated VGAM FOLDED PRECURSOR RNA, of VGAM205 are further described hereinbelow with reference to Table 1.

Nucleotide sequences of host target binding sites, such as BINDING SITE-I, BINDING SITE-II and BINDING SITE-III of FIG. 1, found on VGAM205 host target RNA, and schematic representation of the complementarity of each of these host target binding sites to VGAM205 RNA, herein designated VGAM RNA, are described hereinbelow with reference to Table 2.

As mentioned hereinabove with reference to FIG. 1, a function of VGAM205 gene, herein designated VGAM is inhibition of expression of VGAM205 target genes. It is appreciated that specific functions, and accordingly utilities, of VGAM205 correlate with, and may be deduced from, the identity of the target genes which VGAM205 binds and inhibits, and the function of these target genes, as elaborated hereinbelow.

Breast Cancer 1, Early Onset (BRCA1, Accession NM_{—}007306) is a VGAM205 host target gene. BRCA1 BINDING SITE is HOST TARGET binding site found in the 3′ untranslated region of mRNA encoded by BRCA1, corresponding to a HOST TARGET binding site such as BINDING SITE I, BINDING SITE II or BINDING SITE III. Table 2 illustrates the complementarity of the nucleotide sequences of BRCA1 BINDING SITE, designated SEQ ID:1390, to the nucleotide sequence of VGAM205 RNA, herein designated VGAM RNA, also designated SEQ ID:540.

A function of VGAM205 is therefore inhibition of Breast Cancer 1, Early Onset (BRCA1, Accession NM_{—}007306). Accordingly, utilities of VGAM205 include diagnosis, prevention and treatment of diseases and clinical conditions associated with BRCA1. Chromosome 18 Open Reading Frame 1 (C18orf1, Accession XM_{—}012723) is another VGAM205 host target gene. C18orf1 BINDING SITE is HOST TARGET binding site found in the 3′ untranslated region of mRNA encoded by C18orf1, corresponding to a HOST TARGET binding site such as BINDING SITE I, BINDING SITE II or BINDING SITE III. Table 2 illustrates the complementarity of the nucleotide sequences of C18orf1 BINDING SITE, designated SEQ ID:2562, to the nucleotide sequence of VGAM205 RNA, herein designated VGAM RNA, also designated SEQ ID:540.

Another function of VGAM205 is therefore inhibition of Chromosome 18 Open Reading Frame 1 (C18orf1, Accession XM_{—}012723), a gene which displays selective expression, regulated spatially and temporally. Accordingly, utilities of VGAM205 include diagnosis, prevention and treatment of diseases and clinical conditions associated with C18orf1. The function of C18orf1 and its association with various diseases and clinical conditions, has been established by previous studies, as described hereinabove with reference to VGAM156. Centaurin, Delta 1 (CENTD1, Accession NM_{—}015230) is another VGAM205 host target gene. CENTD1 BINDING SITE1 and CENTD1 BINDING SITE2 are HOST TARGET binding sites found in untranslated regions of mRNA encoded by CENTD1, corresponding to HOST TARGET binding sites such as BINDING SITE I, BINDING SITE II or BINDING SITE III. Table 2 illustrates the complementarity of the nucleotide sequences of CENTD1 BINDING SITE1 and CENTD1 BINDING SITE2, designated SEQ ID: 1614 and SEQ ID:2473 respectively, to the nucleotide sequence of VGAM205 RNA, herein designated VGAM RNA, also designated SEQ ID:540.

Another function of VGAM205 is therefore inhibition of Centaurin, Delta 1 (CENTD1, Accession NM_{—}015230), a gene which is involved in cell signaling/communication. Accordingly, utilities of VGAM205 include diagnosis, prevention and treatment of diseases and clinical conditions associated with CENTD1. The function of CENTD1 has been established by previous studies. By screening brain cDNAs for the potential to encode proteins that are at least 50 kD, Nagase et al. (1998) identified a partial cDNA encoding CENTD1, which they called KIAA0580. The protein was predicted to be involved in cell signaling/communication. RT-PCR analysis detected expression of KIAA0580 in all tissues tested except skeletal muscle. By searching sequence databases, followed by 5-prime RACE, Miura et al. (2002) obtained full-length cDNAs encoding CENTD1 and CENTD2 (OMIM Ref. No. 606646), which they called ARAP2 and ARAP1, respectively. Like ARAP1, the 1,704-amino acid ARAP2 protein contains ARF-GAP (see OMIM Ref. No. 103180), RHO-GAP (see OMIM Ref. No. 602732), ankyrin repeat (see OMIM Ref. No. 605787), RAS (OMIM Ref. No. 190020)-associating, and 5 pleckstrin (OMIM Ref. No. 173570) homology (PH) domains. However, unlike ARAP1, ARAP2 also has a sterile alpha motif (SAM) domain like that found in EphA receptor (see OMIM Ref. No. 179610) and a region of homology to the switch-2 domain of RAB13 (OMIM Ref. No. 602672). The RHO-GAP domain of ARAP2 lacks the predicted catalytic arginine and is therefore unlikely to have RHO-GAP activity. Northern blot analysis showed that ARAP2 is much more variably expressed than ARAP1. The highest ARAP2 expression levels were in brain, thymus, spleen, kidney, peripheral blood leukocytes, lymph node, spinal cord, and thyroid. Two messages at 7.5 and 11 kb were found in brain, and 7.5- and 8.5-kb messages were found in thymus, spleen, kidney, peripheral blood leukocytes, and lymph node. Of the hematopoietic tissues examined, only bone marrow did not show ARAP2 expression.

Full details of the abovementioned studies are described in the following publications, the disclosure of which are hereby incorporated by reference:
Miura, K.; Jacques, K. M.; Stauffer, S.; Kubosaki, A.; Zhu, K.; Hirsch, D. S.; Resau, J.; Zheng, Y.; Randazzo, P. A.: ARAP1: a point of convergence for Arf and Rho signaling. Molec. Cell 9: 109-119, 2002.; and
Nagase, T.; Ishikawa, K.; Miyajima, N.; Tanaka, A.; Kotani, H.; Nomura, N.; Ohara, O.: Prediction of the coding sequences of unidentified human genes. IX. The complete sequences of 10.

Further studies establishing the function and utilities of CENTD1 are found in John Hopkins OMIM database record ID 606645, and in sited publications numbered 141 and 1563 listed in the bibliography section hereinbelow, which are also hereby incorporated by reference. EPB72 (Accession NM_{—}004099) is another VGAM205 host target gene. EPB72 BINDING SITE is HOST TARGET binding site found in the 3′ untranslated region of mRNA encoded by EPB72, corresponding to a HOST TARGET binding site such as BINDING SITE I, BINDING SITE II or BINDING SITE III. Table 2 illustrates the complementarity of the nucleotide sequences of EPB72 BINDING SITE, designated SEQ ID: 1086, to the nucleotide sequence of VGAM205 RNA, herein designated VGAM RNA, also designated SEQ ID:540.

Another function of VGAM205 is therefore inhibition of EPB72 (Accession NM_{—}004099), a gene which may regulate cation conductance. Accordingly, utilities of VGAM205 include diagnosis, prevention and treatment of diseases and clinical conditions associated with EPB72. The function of EPB72 has been established by previous studies. Erythrocyte surface protein band 7.2 is a 29,000-kD integral membrane protein that is exposed on the cytoplasmic surface of the membrane and is susceptible to phosphorylation by a cAMP-dependent protein kinase. Deficiency of this protein in red cells is responsible for stomatocytosis (OMIM Ref. No. 185000). The same protein can be demonstrated in human cell lines of epithelial and lymphoid origin, notably in HeLa cells. Hiebl-Dirschmied et al. (1991), therefore, could screen HeLa cell cDNA expression libraries with antibodies to the protein in order to isolate cDNA clones, determine the nucleotide sequence, and study the structure of the protein. HeLa and bone marrow cell-derived sequences were identical, except for one nucleotide; the deduced sequence of 287 amino acids was confirmed by sequence identity with peptides of the erythroid protein. Structural analysis assigned band 7 protein to the type Ib transmembrane proteins. Westberg et al. (1993) used a cDNA clone coding for stomatin to determine the chromosomal localization of the EPB72 gene. They assigned the gene to human chromosome 9 by Southern blot analysis of somatic cell hybrids. By analysis of hybrid cells containing only parts of chromosome 9, they regionalized the assignment to 9q34.1, proximal to the breakpoint that creates the Philadelphia chromosome of chronic myeloid leukemia (CML; 151410) and, therefore, proximal to the Abelson oncogene (OMIM Ref. No. 189980). Using fluorescence in situ hybridization, Gallagher et al. (1993) likewise mapped the EPB72 gene to 9q33-q34. They showed that EPB72 was not translocated with the 3-prime end of the ABL gene in the Philadelphia chromosome, suggesting that the EPB72 gene is centromeric to the ABL gene. Pilz et al. (1994) demonstrated that the homologous gene is located on mouse chromosome 2. To gain additional insight into the structure and function of this protein, Gallagher et al. (1995) cloned the mouse band 7.2b cDNA and studied its tissue-specific expression. They isolated 2,873 bp of cDNA with an open reading frame of 852 bp. The predicted protein was 284 amino acids with a molecular weight of 31 kD. They detected a wide pattern of expression, with high levels of mRNA in heart, liver, skeletal muscle, and testis but low levels in lung, brain, and spleen. Using fluorescence in situ hybridization, the murine band 7.2b gene was mapped to chromosome 2, at the border of the distal region of 2B and proximal region of Cl, syntenic to 9q, the location of the human homolog. Models of the predicted protein structure showed a short NH2-terminal head, a strongly hydrophobic 28-amino acid stretch presumably encoding a single membrane-spanning domain, and a large domain composed of beta sheet and alpha helix. Database searching showed no significant homology of other known proteins to either the human or the murine band 7.2b. Gallagher and Forget (1995) determined the sequence of the full-length human band 7.2b cDNA, characterized the genomic structure of the EPB72 gene, studied its pattern of expression in different tissues, and characterized the promoter of the gene. The gene is composed of 7 exons distributed over 40 kb of DNA. Its promoter was identified as lacking a TATA box and to be GC-rich. It directed high-level expression of a reporter gene in both erythroid and non-erythroid cells. Unfried et al. (1995) showed that the human EPB72 gene contains 7 exons spanning about 30 kb. Two polyadenylation signals were found in the 3-prime UTR accounting for the 3.2- and 3.3-kb RNAs that are observed in Northern blots. Animal model experiments lend further support to the function of EPB72. To examine the relationship between erythrocyte membrane protein 7.2b deficiency and the hemolytic anemia of human hereditary stomatocytosis, Zhu et al. (1999) created 7.2b knockout mice by standard gene targeting approaches. Despite a complete absence of protein 7.2b in homozygous knockout mice, there was no hemolytic anemia, and mouse red blood cells were normal in morphology, cell indices, hydration status, monovalent cation content, and ability to translocate lipids. Thus, their experiments suggested that 7.2b deficiency plays no direct role in the etiology of stomatocytosis and excluded any role of this protein as a mediator of cation transport in red blood cells.

It is appreciated that the abovementioned animal model for EPB72 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Zhu, Y.; Paszty, C.; Turetsky, T.; Tsai, S.; Kuypers, F. A.; Lee, G.; Cooper, P.; Gallagher, P. G.; Stevens, M. E.; Rubin, E.; Mohandas, N.; Mentzer, W. C.: Stomatocytosis is absent in ‘stomatin’-deficient murine red blood cells. Blood 93: 2404-2410, 1999.; and
Zhu, Y.; Paszty, C.; Turetsky, T.; Tsai, S.; Kuypers, F. A.; Lee, G.; Cooper, P.; Gallagher, P. G.; Stevens, M. E.; Rubin, E.; Mohandas, N.; Mentzer, W. C.: Stomatocytosis is absent in.

Further studies establishing the function and utilities of EPB72 are found in John Hopkins OMIM database record ID 133090, and in sited publications numbered 960-967 listed in the bibliography section hereinbelow, which are also hereby incorporated by reference. Guanylate Binding Protein 1, Interferon-inducible, 67 kDa (GBP1, Accession NM_{—}002053) is another VGAM205 host target gene. GBP1 BINDING SITE is HOST TARGET binding site found in the 3′ untranslated region of mRNA encoded by GBP1, corresponding to a HOST TARGET binding site such as BINDING SITE I, BINDING SITE II or BINDING SITE III. Table 2 illustrates the complementarity of the nucleotide sequences of GBP1 BINDING SITE, designated SEQ ID:894, to the nucleotide sequence of VGAM205 RNA, herein designated VGAM RNA, also designated SEQ ID:540.

Another function of VGAM205 is therefore inhibition of Guanylate Binding Protein 1, Interferon-inducible, 67 kDa (GBP1, Accession NM_{—}002053), a gene which specifically binds guanylate nucleotides (GMP, GDP and GTP). Accordingly, utilities of VGAM205 include diagnosis, prevention and treatment of diseases and clinical conditions associated with GBP1. The function of GBP1 has been established by previous studies. Interferons are cytokines that have antiviral effects and inhibit tumor cell proliferation. They induce a large number of genes in their target cells, including those coding for the guanylate-binding proteins (GBPs). GBPs are characterized by their ability to specifically bind guanine nucleotides (GMP, GDP, and GTP) and are distinguished from the GTP-binding proteins by the presence of 2 binding motifs rather than 3. Cheng et al. (1991) cloned the cDNAs for GBP1 (a 67-kD protein) and GBP2 (partial cDNA; 600412). Strehlow et al. (1994) identified the human GBP1 gene and showed that it contains 11 exons. By use of somatic cell hybrid DNAS, they mapped the gene to human chromosome 1. A mouse homolog of GBP1 has been mapped to the distal region of mouse chromosome 3 (Prochazka et al., 1985). Strehlow et al. (1994) also identified and partially characterized a third novel member of the family (GBP3; 600413) which shows significant sequence similarity to both GBP1 and GBP2. The putative window of embryo implantation in the human opens between days 19 to 24 of the menstrual cycle. A major challenge in the study of human reproduction is to identify the molecular signals that participate in the establishment of this critical receptive phase in the context of the natural cycle. Toward this goal, Kumar et al. (2001) analyzed human endometrial biopsies at various days of the menstrual cycle by mRNA differential display. They isolated several cDNAs representing genes that are either up- or downregulated within the putative window of implantation. They identified one of these genes as GBP1, which possesses GTPase activity. Analysis of endometrial biopsies by Northern blot and RT-PCR demonstrated that GBP1 mRNA is specifically induced at the mid-secretory phase of the menstrual cycle. In situ hybridization analysis revealed that GBP1 mRNA expression is localized in the glandular epithelial cells as well as in the stroma in the immediate vicinity of the glands. The authors concluded that its unique expression overlapping the putative window of implantation suggests that GBP1 may serve as a useful marker of uterine receptivity in the human.

Full details of the abovementioned studies are described in the following publications, the disclosure of which are hereby incorporated by reference:
Cheng, Y.-S. E.; Patterson, C. E.; Staeheli, P.: Interferon-induced guanylate-binding proteins lack an N(T)KXD consensus motif and bind GMP in addition to GDP and GTP. Molec. Cell. Biol. 11: 4717-4725, 1991.; and
Kumar, S.; Li, Q.; Dua, A.; Ying, Y.-K.; Bagchi, M. K.; Bagchi, I. C.: Messenger ribonucleic acid encoding interferon-inducible guanylate binding protein 1 is induced in human endometri.

Further studies establishing the function and utilities of GBP1 are found in John Hopkins OMIM database record ID 600411, and in sited publications numbered 2214-2217 listed in the bibliography section hereinbelow, which are also hereby incorporated by reference. Matrix Metalloproteinase 2 (gelatinase A, 72 kDa gelatinase, 72 kDa type IV collagenase) (MMP2, Accession NM_{—}004530) is another VGAM205 host target gene. MMP2 BINDING SITE is HOST TARGET binding site found in the 3′ untranslated region of mRNA encoded by MMP2, corresponding to a HOST TARGET binding site such as BINDING SITE I, BINDING SITE II or BINDING SITE III. Table 2 illustrates the complementarity of the nucleotide sequences of MMP2 BINDING SITE, designated SEQ ID:1124, to the nucleotide sequence of VGAM205 RNA, herein designated VGAM RNA, also designated SEQ ID:540.

Another function of VGAM205 is therefore inhibition of Matrix Metalloproteinase 2 (gelatinase A, 72 kDa gelatinase, 72 kDa type IV collagenase) (MMP2, Accession NM_{—}004530). Accordingly, utilities of VGAM205 include diagnosis, prevention and treatment of diseases and clinical conditions associated with MMP2. Pleckstrin Homology, Sec7 and Coiled/coil Domains 4 (PSCD4, Accession NM_{—}013385) is another VGAM205 host target gene. PSCD4 BINDING SITE is HOST TARGET binding site found in the 3′ untranslated region of mRNA encoded by PSCD4, corresponding to a HOST TARGET binding site such as BINDING SITE I, BINDING SITE II or BINDING SITE III. Table 2 illustrates the complementarity of the nucleotide sequences of PSCD4 BINDING SITE, designated SEQ ID: 1447, to the nucleotide sequence of VGAM205 RNA, herein designated VGAM RNA, also designated SEQ ID:540.

Another function of VGAM205 is therefore inhibition of Pleckstrin Homology, Sec7 and Coiled/coil Domains 4 (PSCD4, Accession NM_{—}013385), a gene which promotes guanine-nucleotide exchange on arf1 and arf5. Accordingly, utilities of VGAM205 include diagnosis, prevention and treatment of diseases and clinical conditions associated with PSCD4. The function of PSCD4 has been established by previous studies. ADP-ribosylation factors, or ARFS (see OMIM Ref. No. ARF1; 103180), are small GTP-binding proteins within the Ras super family that regulate vesicle trafficking in eukaryotic cells. ARF1 recruits coat proteins (e.g., COPA; 601924) to membranes on the cytoplasmic face of the Golgi apparatus. The PSCD proteins (e.g., PSCD1; 182115), a family of proteins containing a C-terminal pleckstrin homology (PH) domain and a central 200-amino acid region similar to a domain within the yeast Sec7 protein, which is required for vesicular traffic of polypeptides through the Golgi, function as guanine-nucleotide exchange factors (GEFs) for ARFs. Klarlund et al. (1997) identified a cDNA encoding mouse Grp1 (general receptor for phosphoinositides-1) by screening mouse adipocyte and brain cDNA expression libraries with phosphoinositide probes. By searching an EST database for sequences similar to mouse brain Grp1, followed by PCR and screening of a human blood cDNA library, Venkateswarlu et al. (1998) obtained a cDNA encoding PSCD3, which they called GRP1. Sequence analysis showed that the predicted 399-amino acid PSCD3 protein contains a 39-amino acid coiled-coil domain, a 172-amino acid Sec7 domain, and a 118-amino acid PH domain. PSCD3 shares 82.7% and 79.5% amino acid identity with PSCD1 and PSCD2 (OMIM Ref. No. 602488), respectively, as well as 98.8% identity with mouse Grp1. By Scatchard and mutational analyses, Venkateswarlu et al. (1998) determined that PSCD3 binds via its PH domain to the inositol head group of phosphatidylinositol 3,4,5-triphosphate with high affinity. Confocal laser microscopy demonstrated that stimulation of cells with either epidermal growth factor (EGF; 131530) or nerve growth factor (NGF; 162030) results in PH domain-dependent translocation of PSCD3 from the cytosol to the plasma membrane. The translocation was rapid and transient with EGF, whereas NGF mediated a relatively longer translocation. By searching an EST database for Sec7 domain-related sequences and by screening a placenta cDNA library, Franco et al. (1998) isolated a cDNA encoding PSCD3, which they called ARNO3. Northern blot analysis revealed that PSCD3, in contrast to the ubiquitously expressed PSCD1 and PSCD2, is expressed as a 4.5-kb transcript that is almost absent from liver, thymus, and peripheral blood lymphocytes. Franco et al. (1998) found that PSCD3, like PSCD1 and PSCD2, shows GEF activity, mediated by the Sec7 domain, towards ARF1 but not ARF6 (OMIM Ref. No. 600464). Immunofluorescence microscopy indicated that overexpression of PSCD3 induces major morphologic alterations of the Golgi apparatus, including redistribution of Golgi resident proteins and the coat protein COPB (OMIM Ref. No. 600959). Lietzke et al. (2000) and Ferguson et al. (2000) determined the structure of the GRP1 PH domain in the unliganded form and bound to inositol 1,3,4,5-tetraphosphate. Lietzke et al. (2000) found that a novel mode of phosphoinositide recognition involving a 20-residue insertion within the beta-6/beta-7 loop explains the unusually high specificity of the GRP1 PH domain and the promiscuous 3-phosphoinositide binding typical of several other PH domains, including that of protein kinase B (AKT1; 164730). By comparing the GRP1 PH domain to other PH domains, general determinants of 3-phosphoinositide recognition and specificity could be deduced.

Full details of the abovementioned studies are described in the following publications, the disclosure of which are hereby incorporated by reference:
Ferguson, K. M.; Kavran, J. M.; Sankaran, V. G.; Fournier, E.; Isakoff, S. J.; Skolnik, E. Y.; Lemmon, M. A.: Structural basis for discrimination of 3-phosphoinositides by pleckstrin homology domains. Molec. Cell 6: 373-384, 2000.; and
Franco, M.; Boretto, J.; Robineau, S.; Monier, S.; Goud, B.; Chardin, P.; Chavrier, P.: ARNO3, a Sec7-domain guanine nucleotide exchange factor for ADP ribosylation factor 1, is invol.

Further studies establishing the function and utilities of PSCD4 are found in John Hopkins OMIM database record ID 606514, and in sited publications numbered 2451 listed in the bibliography section hereinbelow, which are also hereby incorporated by reference. Solute Carrier Family 7 (cationic amino acid transporter, y+ system), Member 6 (SLC7A6, Accession NM_{—}003983) is another VGAM205 host target gene. SLC7A6 BINDING SITE is HOST TARGET binding site found in the 3′ untranslated region of mRNA encoded by SLC7A6, corresponding to a HOST TARGET binding site such as BINDING SITE I, BINDING SITE II or BINDING SITE III. Table 2 illustrates the complementarity of the nucleotide sequences of SLC7A6 BINDING SITE, designated SEQ ID: 1077, to the nucleotide sequence of VGAM205 RNA, herein designated VGAM RNA, also designated SEQ ID:540.

Another function of VGAM205 is therefore inhibition of Solute Carrier Family 7 (cationic amino acid transporter, y+ system), Member 6 (SLC7A6, Accession NM_{—}003983), a gene which is involved in mediating amino acid transport. Accordingly, utilities of VGAM205 include diagnosis, prevention and treatment of diseases and clinical conditions associated with SLC7A6. The function of SLC7A6 and its association with various diseases and clinical conditions, has been established by previous studies, as described hereinabove with reference to VGAM48. X Kell Blood Group Precursor-related, Y-linked (XKRY, Accession NM_{—}004677) is another VGAM205 host target gene. XKRY BINDING SITE1 and XKRY BINDING SITE2 are HOST TARGET binding sites found in untranslated regions of mRNA encoded by XKRY, corresponding to HOST TARGET binding sites such as BINDING SITE I, BINDING SITE II or BINDING SITE III. Table 2 illustrates the complementarity of the nucleotide sequences of XKRY BINDING SITE1 and XKRY BINDING SITE2, designated SEQ ID:1142 and SEQ ID:2551 respectively, to the nucleotide sequence of VGAM205 RNA, herein designated VGAM RNA, also designated SEQ ID:540.

Another function of VGAM205 is therefore inhibition of X Kell Blood Group Precursor-related, Y-linked (XKRY, Accession NM_{—}004677). Accordingly, utilities of VGAM205 include diagnosis, prevention and treatment of diseases and clinical conditions associated with XKRY. Chromosome 3 Open Reading Frame 4 (C3orf4, Accession NM_{—}019895) is another VGAM205 host target gene. C3orf4 BINDING SITE is HOST TARGET binding site found in the 3′ untranslated region of mRNA encoded by C3orf4, corresponding to a HOST TARGET binding site such as BINDING SITE I, BINDING SITE II or BINDING SITE III. Table 2 illustrates the complementarity of the nucleotide sequences of C3orf4 BINDING SITE, designated SEQ ID:1890, to the nucleotide sequence of VGAM205 RNA, herein designated VGAM RNA, also designated SEQ ID:540.

Another function of VGAM205 is therefore inhibition of Chromosome 3 Open Reading Frame 4 (C3orf4, Accession NM_{—}019895). Accordingly, utilities of VGAM205 include diagnosis, prevention and treatment of diseases and clinical conditions associated with C3orf4. FLJ10483 (Accession NM_{—}018108) is another VGAM205 host target gene. FLJ10483 BINDING SITE is HOST TARGET binding site found in the 3′ untranslated region of mRNA encoded by FLJ10483, corresponding to a HOST TARGET binding site such as BINDING SITE I, BINDING SITE II or BINDING SITE III. Table 2 illustrates the complementarity of the nucleotide sequences of FLJ10483 BINDING SITE, designated SEQ ID: 1781, to the nucleotide sequence of VGAM205 RNA, herein designated VGAM RNA, also designated SEQ ID:540.

Another function of VGAM205 is therefore inhibition of FLJ10483 (Accession NM_{—}018108). Accordingly, utilities of VGAM205 include diagnosis, prevention and treatment of diseases and clinical conditions associated with FLJ10483. FLJ12568 (Accession NM_{—}024993) is another VGAM205 host target gene. FLJ12568 BINDING SITE is HOST TARGET binding site found in the 3′ untranslated region of mRNA encoded by FLJ12568, corresponding to a HOST TARGET binding site such as BINDING SITE I, BINDING SITE II or BINDING SITE III. Table 2 illustrates the complementarity of the nucleotide sequences of FLJ12568 BINDING SITE, designated SEQ ID:2122, to the nucleotide sequence of VGAM205 RNA, herein designated VGAM RNA, also designated SEQ ID:540.

Another function of VGAM205 is therefore inhibition of FLJ12568 (Accession NM_{—}024993). Accordingly, utilities of VGAM205 include diagnosis, prevention and treatment of diseases and clinical conditions associated with FLJ12568. FLJ20340 (Accession NM_{—}017773) is another VGAM205 host target gene. FLJ20340 BINDING SITE is HOST TARGET binding site found in the 3′ untranslated region of mRNA encoded by FLJ20340, corresponding to a HOST TARGET binding site such as BINDING SITE I, BINDING SITE II or BINDING SITE III. Table 2 illustrates the complementarity of the nucleotide sequences of FLJ20340 BINDING SITE, designated SEQ ID:1750, to the nucleotide sequence of VGAM205 RNA, herein designated VGAM RNA, also designated SEQ ID:540.

Another function of VGAM205 is therefore inhibition of FLJ20340 (Accession NM_{—}017773). Accordingly, utilities of VGAM205 include diagnosis, prevention and treatment of diseases and clinical conditions associated with FLJ20340. FLJ20727 (Accession NM_{—}017944) is another VGAM205 host target gene. FLJ20727 BINDING SITE is HOST TARGET binding site found in the 5′ untranslated region of mRNA encoded by FLJ20727, corresponding to a HOST TARGET binding site such as BINDING SITE I, BINDING SITE II or BINDING SITE III. Table 2 illustrates the complementarity of the nucleotide sequences of FLJ20727 BINDING SITE, designated SEQ ID:1769, to the nucleotide sequence of VGAM205 RNA, herein designated VGAM RNA, also designated SEQ ID:540.

Another function of VGAM205 is therefore inhibition of FLJ20727 (Accession NM_{—}017944). Accordingly, utilities of VGAM205 include diagnosis, prevention and treatment of diseases and clinical conditions associated with FLJ20727. FLJ20736 (Accession NM_{—}017948) is another VGAM205 host target gene. FLJ20736 BINDING SITE is HOST TARGET binding site found in the 3′ untranslated region of mRNA encoded by FLJ20736, corresponding to a HOST TARGET binding site such as BINDING SITE I, BINDING SITE II or BINDING SITE III. Table 2 illustrates the complementarity of the nucleotide sequences of FLJ20736 BINDING SITE, designated SEQ ID:1770, to the nucleotide sequence of VGAM205 RNA, herein designated VGAM RNA, also designated SEQ ID:540.

Another function of VGAM205 is therefore inhibition of FLJ20736 (Accession NM_{—}017948). Accordingly, utilities of VGAM205 include diagnosis, prevention and treatment of diseases and clinical conditions associated with FLJ20736. Phosphodiesterase 1C, Calmodulin-dependent 70 kDa (PDE1C, Accession NM_{—}005020) is another VGAM205 host target gene. PDE1C BINDING SITE is HOST TARGET binding site found in the 3′ untranslated region of mRNA encoded by PDE1C, corresponding to a HOST TARGET binding site such as BINDING SITE I, BINDING SITE II or BINDING SITE III. Table 2 illustrates the complementarity of the nucleotide sequences of PDE1C BINDING SITE, designated SEQ ID:1172, to the nucleotide sequence of VGAM205 RNA, herein designated VGAM RNA, also designated SEQ ID:540.

Another function of VGAM205 is therefore inhibition of Phosphodiesterase 1C, Calmodulin-dependent 70 kDa (PDE1C, Accession NM_{—}005020). Accordingly, utilities of VGAM205 include diagnosis, prevention and treatment of diseases and clinical conditions associated with PDE1C. Transducer of ERBB2, 2 (TOB2, Accession XM_{—}170995) is another VGAM205 host target gene. TOB2 BINDING SITE is HOST TARGET binding site found in the 5′ untranslated region of mRNA encoded by TOB2, corresponding to a HOST TARGET binding site such as BINDING SITE I, BINDING SITE II or BINDING SITE III. Table 2 illustrates the complementarity of the nucleotide sequences of TOB2 BINDING SITE, designated SEQ ID:3680, to the nucleotide sequence of VGAM205 RNA, herein designated VGAM RNA, also designated SEQ ID:540.

Another function of VGAM205 is therefore inhibition of Transducer of ERBB2, 2 (TOB2, Accession XM_{—}170995). Accordingly, utilities of VGAM205 include diagnosis, prevention and treatment of diseases and clinical conditions associated with TOB2. TSC22 (Accession NM_{—}006022) is another VGAM205 host target gene. TSC22 BINDING SITE is HOST TARGET binding site found in the 3′ untranslated region of mRNA encoded by TSC22, corresponding to a HOST TARGET binding site such as BINDING SITE I, BINDING SITE II or BINDING SITE III. Table 2 illustrates the complementarity of the nucleotide sequences of TSC22 BINDING SITE, designated SEQ ID:1264, to the nucleotide sequence of VGAM205 RNA, herein designated VGAM RNA, also designated SEQ ID:540.

Another function of VGAM205 is therefore inhibition of TSC22 (Accession NM_{—}006022). Accordingly, utilities of VGAM205 include diagnosis, prevention and treatment of diseases and clinical conditions associated with TSC22. TSP-NY (Accession NM_{—}032573) is another VGAM205 host target gene. TSP-NY BINDING SITE is HOST TARGET binding site found in the 3′ untranslated region of mRNA encoded by TSP-NY, corresponding to a HOST TARGET binding site such as BINDING SITE I, BINDING SITE II or BINDING SITE III. Table 2 illustrates the complementarity of the nucleotide sequences of TSP-NY BINDING SITE, designated SEQ ID:2262, to the nucleotide sequence of VGAM205 RNA, herein designated VGAM RNA, also designated SEQ ID:540.

Another function of VGAM205 is therefore inhibition of TSP-NY (Accession NM_{—}032573). Accordingly, utilities of VGAM205 include diagnosis, prevention and treatment of diseases and clinical conditions associated with TSP-NY. TUCAN (Accession NM_{—}014959) is another VGAM205 host target gene. TUCAN BINDING SITE is HOST TARGET binding site found in the 3′ untranslated region of mRNA encoded by TUCAN, corresponding to a HOST TARGET binding site such as BINDING SITE I, BINDING SITE II or BINDING SITE III. Table 2 illustrates the complementarity of the nucleotide sequences of TUCAN BINDING SITE, designated SEQ ID: 1598, to the nucleotide sequence of VGAM205 RNA, herein designated VGAM RNA, also designated SEQ ID:540.

Another function of VGAM205 is therefore inhibition of TUCAN (Accession NM_{—}014959). Accordingly, utilities of VGAM205 include diagnosis, prevention and treatment of diseases and clinical conditions associated with TUCAN. LOC138241 (Accession XM_{—}059957) is another VGAM205 host target gene. LOC138241 BINDING SITE is HOST TARGET binding site found in the 5′ untranslated region of mRNA encoded by LOC138241, corresponding to a HOST TARGET binding site such as BINDING SITE I, BINDING SITE II or BINDING SITE III. Table 2 illustrates the complementarity of the nucleotide sequences of LOC138241 BINDING SITE, designated SEQ ID:3019, to the nucleotide sequence of VGAM205 RNA, herein designated VGAM RNA, also designated SEQ ID:540.

Another function of VGAM205 is therefore inhibition of LOC138241 (Accession XM_{—}059957). Accordingly, utilities of VGAM205 include diagnosis, prevention and treatment of diseases and clinical conditions associated with LOC138241. LOC152345 (Accession XM_{—}087442) is another VGAM205 host target gene. LOC152345 BINDING SITE is HOST TARGET binding site found in the 3′ untranslated region of mRNA encoded by LOC152345, corresponding to a HOST TARGET binding site such as BINDING SITE I, BINDING SITE II or BINDING SITE III. Table 2 illustrates the complementarity of the nucleotide sequences of LOC152345 BINDING SITE, designated SEQ ID:3170, to the nucleotide sequence of VGAM205 RNA, herein designated VGAM RNA, also designated SEQ ID:540.

Another function of VGAM205 is therefore inhibition of LOC152345 (Accession XM_{—}087442). Accordingly, utilities of VGAM205 include diagnosis, prevention and treatment of diseases and clinical conditions associated with LOC152345. LOC154214 (Accession XM_{—}087876) is another VGAM205 host target gene. LOC154214 BINDING SITE is HOST TARGET binding site found in the 5′ untranslated region of mRNA encoded by LOC154214, corresponding to a HOST TARGET binding site such as BINDING SITE I, BINDING SITE II or BINDING SITE III. Table 2 illustrates the complementarity of the nucleotide sequences of LOC154214 BINDING SITE, designated SEQ ID:3189, to the nucleotide sequence of VGAM205 RNA, herein designated VGAM RNA, also designated SEQ ID:540.

Another function of VGAM205 is therefore inhibition of LOC154214 (Accession XM_{—}087876). Accordingly, utilities of VGAM205 include diagnosis, prevention and treatment of diseases and clinical conditions associated with LOC154214. LOC154790 (Accession XM_{—}088044) is another VGAM205 host target gene. LOC154790 BINDING SITE is HOST TARGET binding site found in the 5′ untranslated region of mRNA encoded by LOC154790, corresponding to a HOST TARGET binding site such as BINDING SITE I, BINDING SITE II or BINDING SITE III. Table 2 illustrates the complementarity of the nucleotide sequences of LOC154790 BINDING SITE, designated SEQ ID:3192, to the nucleotide sequence of VGAM205 RNA, herein designated VGAM RNA, also designated SEQ ID:540.

Another function of VGAM205 is therefore inhibition of LOC154790 (Accession XM_{—}088044). Accordingly, utilities of VGAM205 include diagnosis, prevention and treatment of diseases and clinical conditions associated with LOC154790. LOC158427 (Accession NM_{—}139246) is another VGAM205 host target gene. LOC158427 BINDING SITE is HOST TARGET binding site found in the 3′ untranslated region of mRNA encoded by LOC158427, corresponding to a HOST TARGET binding site such as BINDING SITE I, BINDING SITE II or BINDING SITE III. Table 2 illustrates the complementarity of the nucleotide sequences of LOC158427 BINDING SITE, designated SEQ ID:2478, to the nucleotide sequence of VGAM205 RNA, herein designated VGAM RNA, also designated SEQ ID:540.

Another function of VGAM205 is therefore inhibition of LOC158427 (Accession NM_{—}139246). Accordingly, utilities of VGAM205 include diagnosis, prevention and treatment of diseases and clinical conditions associated with LOC158427. LOC161003 (Accession NM_{—}145286) is another VGAM205 host target gene. LOC161003 BINDING SITE is HOST TARGET binding site found in the 5′ untranslated region of mRNA encoded by LOC161003, corresponding to a HOST TARGET binding site such as BINDING SITE I, BINDING SITE II or BINDING SITE III. Table 2 illustrates the complementarity of the nucleotide sequences of LOC161003 BINDING SITE, designated SEQ ID:2520, to the nucleotide sequence of VGAM205 RNA, herein designated VGAM RNA, also designated SEQ ID:540.

Another function of VGAM205 is therefore inhibition of LOC161003 (Accession NM_{—}145286). Accordingly, utilities of VGAM205 include diagnosis, prevention and treatment of diseases and clinical conditions associated with LOC161003. LOC200830 (Accession XM_{—}117287) is another VGAM205 host target gene. LOC200830 BINDING SITE is HOST TARGET binding site found in the 3′ untranslated region of mRNA encoded by LOC200830, corresponding to a HOST TARGET binding site such as BINDING SITE I, BINDING SITE II or BINDING SITE III. Table 2 illustrates the complementarity of the nucleotide sequences of LOC200830 BINDING SITE, designated SEQ ID:3480, to the nucleotide sequence of VGAM205 RNA, herein designated VGAM RNA, also designated SEQ ID:540.

Another function of VGAM205 is therefore inhibition of LOC200830 (Accession XM_{—}117287). Accordingly, utilities of VGAM205 include diagnosis, prevention and treatment of diseases and clinical conditions associated with LOC200830. LOC221421 (Accession XM_{—}166428) is another VGAM205 host target gene. LOC221421 BINDING SITE is HOST TARGET binding site found in the 3′ untranslated region of mRNA encoded by LOC221421, corresponding to a HOST TARGET binding site such as BINDING SITE I, BINDING SITE II or BINDING SITE III. Table 2 illustrates the complementarity of the nucleotide sequences of LOC221421 BINDING SITE, designated SEQ ID:3558, to the nucleotide sequence of VGAM205 RNA, herein designated VGAM RNA, also designated SEQ ID:540.

Another function of VGAM205 is therefore inhibition of LOC221421 (Accession XM_{—}166428). Accordingly, utilities of VGAM205 include diagnosis, prevention and treatment of diseases and clinical conditions associated with LOC221421. LOC257017 (Accession XM_{—}173227) is another VGAM205 host target gene. LOC257017 BINDING SITE is HOST TARGET binding site found in the 5′ untranslated region of mRNA encoded by LOC257017, corresponding to a HOST TARGET binding site such as BINDING SITE I, BINDING SITE II or BINDING SITE III. Table 2 illustrates the complementarity of the nucleotide sequences of LOC257017 BINDING SITE, designated SEQ ID:3736, to the nucleotide sequence of VGAM205 RNA, herein designated VGAM RNA, also designated SEQ ID:540.

Another function of VGAM205 is therefore inhibition of LOC257017 (Accession XM_{—}173227). Accordingly, utilities of VGAM205 include diagnosis, prevention and treatment of diseases and clinical conditions associated with LOC257017. LOC257353 (Accession XM_{—}173264) is another VGAM205 host target gene. LOC257353 BINDING SITE is HOST TARGET binding site found in the 5′ untranslated region of mRNA encoded by LOC257353, corresponding to a HOST TARGET binding site such as BINDING SITE I, BINDING SITE II or BINDING SITE III. Table 2 illustrates the complementarity of the nucleotide sequences of LOC257353 BINDING SITE, designated SEQ ID:3738, to the nucleotide sequence of VGAM205 RNA, herein designated VGAM RNA, also designated SEQ ID:540.

Another function of VGAM205 is therefore inhibition of LOC257353 (Accession XM_{—}173264). Accordingly, utilities of VGAM205 include diagnosis, prevention and treatment of diseases and clinical conditions associated with LOC257353. LOC90459 (Accession XM_{—}031826) is another VGAM205 host target gene. LOC90459 BINDING SITE is HOST TARGET binding site found in the 3′ untranslated region of mRNA encoded by LOC90459, corresponding to a HOST TARGET binding site such as BINDING SITE I, BINDING SITE II or BINDING SITE III. Table 2 illustrates the complementarity of the nucleotide sequences of LOC90459 BINDING SITE, designated SEQ ID:2642, to the nucleotide sequence of VGAM205 RNA, herein designated VGAM RNA, also designated SEQ ID:540.

Another function of VGAM205 is therefore inhibition of LOC90459 (Accession XM_{—}031826). Accordingly, utilities of VGAM205 include diagnosis, prevention and treatment of diseases and clinical conditions associated with LOC90459. FIG. 1 further provides a conceptual description of a novel bioinformatically detected viral gene of the present invention, referred to here as Viral Genomic Address Messenger 206 (VGAM206) viral gene, which modulates expression of respective host target genes thereof, the function and utility of which host target genes is known in the art.

VGAM206 is a novel bioinformatically detected regulatory, non protein coding, viral micro RNA (miRNA) gene. The method by which VGAM206 was detected is described hereinabove with reference to FIGS. 1-8.

VGAM206 gene, herein designated VGAM GENE, is a viral gene contained in the genome of Vaccinia Virus. VGAM206 host target gene, herein designated VGAM HOST TARGET GENE, is a human gene contained in the human genome.

VGAM206 gene encodes a VGAM206 precursor RNA, herein designated VGAM PRECURSOR RNA. Similar to other miRNA genes, and unlike most ordinary genes, VGAM206 precursor RNA does not encode a protein. A nucleotide sequence identical or highly similar to the nucleotide sequence of VGAM206 precursor RNA is designated SEQ ID:192, and is provided hereinbelow with reference to the sequence listing part. Nucleotide sequence SEQ ID:192 is located at position 109361 relative to the genome of Vaccinia Virus.

VGAM206 precursor RNA folds onto itself, forming VGAM206 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM206 folded precursor RNA into VGAM206 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM206 RNA is designated SEQ ID:541, and is provided hereinbelow with reference to the sequence listing part.

VGAM206 host target gene, herein designated VGAM HOST TARGET GENE, encodes a corresponding messenger RNA, VGAM206 host target RNA, herein designated VGAM HOST TARGET RNA. VGAM206 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM206 RNA, herein designated VGAM RNA, binds complementarily to one or more host target binding sites located in untranslated regions of VGAM206 host target RNA, herein designated VGAM HOST TARGET RNA. This complementary binding is due to the fact that the nucleotide sequence of VGAM206 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM206 RNA, herein designated VGAM RNA, may have a different number of host target binding sites in untranslated regions of a VGAM206 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM206 RNA, herein designated VGAM RNA, to host target binding sites on VGAM206 host target RNA, herein designated VGAM HOST TARGET RNA, such as BINDING SITE I, BINDING SITE II and BINDING SITE III, inhibits translation of VGAM206 host target RNA into VGAM206 host target protein, herein designated VGAM HOST TARGET PROTEIN. VGAM host target protein is therefore outlined by a broken line.

It is appreciated that VGAM206 host target gene, herein designated VGAM HOST TARGET GENE, in fact represents a plurality of VGAM206 host target genes. The mRNA of each one of this plurality of VGAM206 host target genes comprises one or more host target binding sites, each having a nucleotide sequence which is at least partly complementary to VGAM206 RNA, herein designated VGAM RNA, and which when bound by VGAM206 RNA causes inhibition of translation of respective one or more VGAM206 host target proteins.

It is further appreciated by one skilled in the art that the mode of translational inhibition illustrated by FIG. 1 with specific reference to translational inhibition exerted by VGAM206 gene, herein designated VGAM GENE, on one or more VGAM206 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM206 is inhibition of expression of host target genes, as part of a novel viral mechanism of attacking a host. Accordingly, utilities of VGAM206 include diagnosis, prevention and treatment of viral infection by Vaccinia Virus. Specific functions, and accordingly utilities, of VGAM206 correlate with, and may be deduced from, the identity of the host target genes which VGAM206 binds and inhibits, and the function of these host target genes, as elaborated hereinbelow.

Nucleotide sequences of the VGAM206 precursor RNA, herein designated VGAM PRECURSOR RNA, and of the ‘diced’ VGAM206 RNA, herein designated VGAM RNA, and a schematic representation of the secondary folding of VGAM206 folded precursor RNA, herein designated VGAM FOLDED PRECURSOR RNA, of VGAM206 are further described hereinbelow with reference to Table 1.

Nucleotide sequences of host target binding sites, such as BINDING SITE-I, BINDING SITE-II and BINDING SITE-III of FIG. 1, found on VGAM206 host target RNA, and schematic representation of the complementarity of each of these host target binding sites to VGAM206 RNA, herein designated VGAM RNA, are described hereinbelow with reference to Table 2.

As mentioned hereinabove with reference to FIG. 1, a function of VGAM206 gene, herein designated VGAM is inhibition of expression of VGAM206 target genes. It is appreciated that specific functions, and accordingly utilities, of VGAM206 correlate with, and may be deduced from, the identity of the target genes which VGAM206 binds and inhibits, and the function of these target genes, as elaborated hereinbelow.

Coxsackie Virus and Adenovirus Receptor (CXADR, Accession NM_{—}001338) is a VGAM206 host target gene. CXADR BINDING SITE is HOST TARGET binding site found in the 3′ untranslated region of mRNA encoded by CXADR, corresponding to a HOST TARGET binding site such as BINDING SITE I, BINDING SITE II or BINDING SITE III. Table 2 illustrates the complementarity of the nucleotide sequences of CXADR BINDING SITE, designated SEQ ID:820, to the nucleotide sequence of VGAM206 RNA, herein designated VGAM RNA, also designated SEQ ID:541.

A function of VGAM206 is therefore inhibition of Coxsackie Virus and Adenovirus Receptor (CXADR, Accession NM_{—}001338), a gene which is a member of the immunoglobulin super family. Accordingly, utilities of VGAM206 include diagnosis, prevention and treatment of diseases and clinical conditions associated with CXADR. The function of CXADR has been established by previous studies. Bergelson et al. (1997) used immunoaffinity chromatography to purify a Coxsackie virus and adenovirus receptor protein, which they termed CAR. Based on the sequences of tryptic peptides, they cloned the corresponding cDNA from a HeLa cell library. The CAR cDNA encodes a predicted 365-amino acid polypeptide that contains a single transmembrane domain and is a member of the immunoglobulin super family. Bergelson et al. (1997) found that Chinese hamster cells bound to labeled Coxsackie viruses B3 and B4 and became susceptible to infection when transfected with CAR cDNA. Myocarditis and dilated cardiomyopathy are common causes of morbidity and mortality in children. Many studies have implicated the enteroviruses and particularly the Coxsackie virus B family as etiologic agents of the acquired forms of these diseases. However, Martin et al. (1994), Griffin et al. (1995), and Pauschinger et al. (1999) showed that the group C adenoviruses are as commonly detected as enteroviruses in the myocardium of children and adults with these diseases. The description of the common Coxsackie virus B and adenovirus receptor offers a partial explanation for the observation that 2 such divergent virus families cause these diseases.

Full details of the abovementioned studies are described in the following publications, the disclosure of which are hereby incorporated by reference:
Bergelson, J. M.; Cunningham, J. A.; Droguett, G.; Kurt-Jones, E. A.; Krithivas, A.; Hong, J. S.; Horwitz, M. S.; Crowell, R. L.; Finberg, R. W.: Isolation of a common receptor for coxsackie B viruses and adenoviruses 2 and 5. Science 275: 1320-1323, 1997.; and
Pauschinger, M.; Bowles, N. E.; Fuentes-Garcia, F. J.; Pham, V.; Kuhl, U.; Schwimmbeck, P. L.; Schultheiss, H.-P.; Towbin, J. A.: Detection of adenoviral genome in the myocardium of adu.

Further studies establishing the function and utilities of CXADR are found in John Hopkins OMIM database record ID 602621, and in sited publications numbered 1929-1935 listed in the bibliography section hereinbelow, which are also hereby incorporated by reference. EPB72 (Accession NM_{—}004099) is another VGAM206 host target gene. EPB72 BINDING SITE is HOST TARGET binding site found in the 3′ untranslated region of mRNA encoded by EPB72, corresponding to a HOST TARGET binding site such as BINDING SITE I, BINDING SITE II or BINDING SITE III. Table 2 illustrates the complementarity of the nucleotide sequences of EPB72 BINDING SITE, designated SEQ ID: 1087, to the nucleotide sequence of VGAM206 RNA, herein designated VGAM RNA, also designated SEQ ID:541.

Another function of VGAM206 is therefore inhibition of EPB72 (Accession NM_{—}004099), a gene which may regulate cation conductance. Accordingly, utilities of VGAM206 include diagnosis, prevention and treatment of diseases and clinical conditions associated with EPB72. The function of EPB72 and its association with various diseases and clinical conditions, has been established by previous studies, as described hereinabove with reference to VGAM205. Fc Fragment of IgA, Receptor For (FCAR, Accession NM_{—}002000) is another VGAM206 host target gene. FCAR BINDING SITE1 through FCAR BINDING SITE7 are HOST TARGET binding sites found in untranslated regions of mRNA encoded by FCAR, corresponding to HOST TARGET binding sites such as BINDING SITE I, BINDING SITE II or BINDING SITE III. Table 2 illustrates the complementarity of the nucleotide sequences of FCAR BINDING SITE1 through FCAR BINDING SITE7, designated SEQ ID:880, SEQ ID:2400, SEQ ID:2401, SEQ ID:2402, SEQ ID:2403, SEQ ID:2404 and SEQ ID:2406 respectively, to the nucleotide sequence of VGAM206 RNA, herein designated VGAM RNA, also designated SEQ ID:541.

Another function of VGAM206 is therefore inhibition of Fc Fragment of IgA, Receptor For (FCAR, Accession NM_{—}002000), a gene which binds to the fc region of immunoglobulins alpha and mediates several functions including cytokine production. Accordingly, utilities of VGAM206 include diagnosis, prevention and treatment of diseases and clinical conditions associated with FCAR. The function of FCAR and its association with various diseases and clinical conditions, has been established by previous studies, as described hereinabove with reference to VGAM170. Nuclear Receptor Coactivator 6 (NCOA6, Accession NM_{—}014071) is another VGAM206 host target gene. NCOA6 BINDING SITE is HOST TARGET binding site found in the 3′ untranslated region of mRNA encoded by NCOA6, corresponding to a HOST TARGET binding site such as BINDING SITE I, BINDING SITE II or BINDING SITE III. Table 2 illustrates the complementarity of the nucleotide sequences of NCOA6 BINDING SITE, designated SEQ ID: 1468, to the nucleotide sequence of VGAM206 RNA, herein designated VGAM RNA, also designated SEQ ID:541.

Another function of VGAM206 is therefore inhibition of Nuclear Receptor Coactivator 6 (NCOA6, Accession NM_{—}014071), a gene which activates gene transcription through ligand-dependent association with coactivators. Accordingly, utilities of VGAM206 include diagnosis, prevention and treatment of diseases and clinical conditions associated with NCOA6. The function of NCOA6 has been established by previous studies. Using the ligand-binding domain of the retinoid X receptor as bait in a yeast 2-hybrid screen to identify potential transcriptional coactivators of nuclear receptors, Lee et al. (1999) isolated a cDNA encoding a nuclear protein-activating signal cointegrator, which they designated ASC2. Sequence analysis of the 2,063 amino acid protein predicted an N-terminal acidic domain, 2 glutamine-rich domains, and a C-terminal serine/threonine-rich domain as well as 2 separate basic potential nuclear localization signal domains and 2 copies of LXXLL motifs that function in ligand-dependent interaction with the AF2 domain of nuclear receptors. Analyzing tissue microarrays by FISH, Lee et al. (1999) found increased ASC2 copy number in 10% of breast cancer specimens and in all breast cancer cell lines. SDS-PAGE analysis showed that amino acids 586-860, which do not include the LXXLL motifs, are the minimum interaction domain with the AF2 domain of numerous nuclear receptors. N-terminal subregions of ASC2, either alone or in conjunction with CBP (OMIM Ref. No. 600140) and SRC1 (NCOA1; 602691), stimulate ligand-dependent transactivation by wildtype nuclear receptors. Microinjection of anti-ASC2 into cells demonstrated abrogation of the transactivation function. Caira et al. (2000) and Mahajan and Samuels (2000) isolated cDNAs encoding a protein identical to ASC2, which they termed nuclear receptor-activating protein 250 kD (OMIM Ref. No. RAP250) and nuclear receptor coregulator (NRC), respectively. Northern blot analysis revealed widespread expression of an approximately 7.5-kb transcript, with a 4.5-kb splice variant detected in testis. Analysis of the testis sequence indicated that it encodes a 1,070 amino acid protein that lacks residues 972-1964 of the full-length sequence (Caira et al., 2000). ASC2 was also found to be widely expressed during ontogeny in rat embryos. Using GST pull-down assays, Caira et al. (2000) determined that the first LXXLL motif (LVNLL, contained in residues 819-1096) but not the second (LSQLL, residues 1491-1495) interacted strongly with nuclear receptors. Mahajan and Samuels (2000) also found that only the first LXXLL domain is functional and that mutation of this sequence abolished transcriptional enhancement.

Full details of the abovementioned studies are described in the following publications, the disclosure of which are hereby incorporated by reference:
Lee, S.-K.; Anzick, S. L.; Choi, J.-E.; Bubendorf, L.; Guan, X.-Y.; Jung, Y.-K.; Kallioniemi, O. P.; Kononen, J.; Trent, J. M.; Azorsa, D.; Jhun, B.-H.; Cheong, J. H.; Lee, Y. C.; Meltzer, P. S.; Lee, J. W.: A nuclear factor, ASC-2, as a cancer-amplified transcriptional coactivator essential for ligand-dependent transactivation by nuclear receptors in vivo. J. Biol. Chem. 274: 34283-34293, 1999.; and
Mahajan, M. A.; Samuels, H. H: A new family of nuclear receptor coregulators that integrate nuclear receptor signaling through CREB-binding protein. Molec. Cell. Biol. 20: 5048-5063.

Further studies establishing the function and utilities of NCOA6 are found in John Hopkins OMIM database record ID 605299, and in sited publications numbered 1678-168 and 2639 listed in the bibliography section hereinbelow, which are also hereby incorporated by reference. Succinate Dehydrogenase Complex, Subunit C, Integral Membrane Protein, 15 kDa (SDHC, Accession XM_{—}045183) is another VGAM206 host target gene. SDHC BINDING SITE is HOST TARGET binding site found in the 3′ untranslated region of mRNA encoded by SDHC, corresponding to a HOST TARGET binding site such as BINDING SITE I, BINDING SITE II or BINDING SITE III. Table 2 illustrates the complementarity of the nucleotide sequences of SDHC BINDING SITE, designated SEQ ID:2856, to the nucleotide sequence of VGAM206 RNA, herein designated VGAM RNA, also designated SEQ ID:541.

Another function of VGAM206 is therefore inhibition of Succinate Dehydrogenase Complex, Subunit C, Integral Membrane Protein, 15 kDa (SDHC, Accession XM_{—}045183). Accordingly, utilities of VGAM206 include diagnosis, prevention and treatment of diseases and clinical conditions associated with SDHC. Cerebellin 1 Precursor (CBLN1, Accession NM_{—}004352) is another VGAM206 host target gene. CBLN1 BINDING SITE is HOST TARGET binding site found in the 5′ untranslated region of mRNA encoded by CBLN1, corresponding to a HOST TARGET binding site such as BINDING SITE I, BINDING SITE II or BINDING SITE III. Table 2 illustrates the complementarity of the nucleotide sequences of CBLN1 BINDING SITE, designated SEQ ID:1101, to the nucleotide sequence of VGAM206 RNA, herein designated VGAM RNA, also designated SEQ ID:541.

Another function of VGAM206 is therefore inhibition of Cerebellin 1 Precursor (CBLN1, Accession NM_{—}004352). Accordingly, utilities of VGAM206 include diagnosis, prevention and treatment of diseases and clinical conditions associated with CBLN1. Cleavage Stimulation Factor, 3′ Pre-RNA, Subunit 2, 64 kDa (CSTF2, Accession NM_{—}001325) is another VGAM206 host target gene. CSTF2 BINDING SITE is HOST TARGET binding site found in the 3′ untranslated region of mRNA encoded by CSTF2, corresponding to a HOST TARGET binding site such as BINDING SITE I, BINDING SITE II or BINDING SITE III. Table 2 illustrates the complementarity of the nucleotide sequences of CSTF2 BINDING SITE, designated SEQ ID:819, to the nucleotide sequence of VGAM206 RNA, herein designated VGAM RNA, also designated SEQ ID:541.

Another function of VGAM206 is therefore inhibition of Cleavage Stimulation Factor, 3′ Pre-RNA, Subunit 2, 64 kDa (CSTF2, Accession NM_{—}001325). Accordingly, utilities of VGAM206 include diagnosis, prevention and treatment of diseases and clinical conditions associated with CSTF2. GS3955 (Accession NM_{—}021643) is another VGAM206 host target gene. GS3955 BINDING SITE is HOST TARGET binding site found in the 5′ untranslated region of mRNA encoded by GS3955, corresponding to a HOST TARGET binding site such as BINDING SITE I, BINDING SITE II or BINDING SITE III. Table 2 illustrates the complementarity of the nucleotide sequences of GS3955 BINDING SITE, designated SEQ ID:1953, to the nucleotide sequence of VGAM206 RNA, herein designated VGAM RNA, also designated SEQ ID:541.

Another function of VGAM206 is therefore inhibition of GS3955 (Accession NM_{—}021643). Accordingly, utilities of VGAM206 include diagnosis, prevention and treatment of diseases and clinical conditions associated with GS3955. KIAA0184 (Accession XM_{—}036865) is another VGAM206 host target gene. KIAA0184 BINDING SITE is HOST TARGET binding site found in the 3′ untranslated region of mRNA encoded by KIAA0184, corresponding to a HOST TARGET binding site such as BINDING SITE I, BINDING SITE II or BINDING SITE III. Table 2 illustrates the complementarity of the nucleotide sequences of KIAA0184 BINDING SITE, designated SEQ ID:2715, to the nucleotide sequence of VGAM206 RNA, herein designated VGAM RNA, also designated SEQ ID:541.

Another function of VGAM206 is therefore inhibition of KIAA0184 (Accession XM_{—}036865). Accordingly, utilities of VGAM206 include diagnosis, prevention and treatment of diseases and clinical conditions associated with KIAA0184. MGC14697 (Accession NM_{—}032747) is another VGAM206 host target gene. MGC14697 BINDING SITE is HOST TARGET binding site found in the 5′ untranslated region of mRNA encoded by MGC14697, corresponding to a HOST TARGET binding site such as BINDING SITE I, BINDING SITE II or BINDING SITE III. Table 2 illustrates the complementarity of the nucleotide sequences of MGC14697 BINDING SITE, designated SEQ ID:2276, to the nucleotide sequence of VGAM206 RNA, herein designated VGAM RNA, also designated SEQ ID:541.

Another function of VGAM206 is therefore inhibition of MGC14697 (Accession NM_{—}032747). Accordingly, utilities of VGAM206 include diagnosis, prevention and treatment of diseases and clinical conditions associated with MGC14697. RRN3 (Accession NM_{—}018427) is another VGAM206 host target gene. RRN3 BINDING SITE is HOST TARGET binding site found in the 3′ untranslated region of mRNA encoded by RRN3, corresponding to a HOST TARGET binding site such as BINDING SITE I, BINDING SITE II or BINDING SITE III. Table 2 illustrates the complementarity of the nucleotide sequences of RRN3 BINDING SITE, designated SEQ ID:1827, to the nucleotide sequence of VGAM206 RNA, herein designated VGAM RNA, also designated SEQ ID:541.

Another function of VGAM206 is therefore inhibition of RRN3 (Accession NM_{—}018427). Accordingly, utilities of VGAM206 include diagnosis, prevention and treatment of diseases and clinical conditions associated with RRN3. WD Repeat Domain 13 (WDR13, Accession NM_{—}017883) is another VGAM206 host target gene. WDR13 BINDING SITE is HOST TARGET binding site found in the 5′ untranslated region of mRNA encoded by WDR13, corresponding to a HOST TARGET binding site such as BINDING SITE I, BINDING SITE II or BINDING SITE III. Table 2 illustrates the complementarity of the nucleotide sequences of WDR13 BINDING SITE, designated SEQ ID:1759, to the nucleotide sequence of VGAM206 RNA, herein designated VGAM RNA, also designated SEQ ID:541.

Another function of VGAM206 is therefore inhibition of WD Repeat Domain 13 (WDR13, Accession NM_{—}017883). Accordingly, utilities of VGAM206 include diagnosis, prevention and treatment of diseases and clinical conditions associated with WDR13. LOC219287 (Accession XM_{—}167994) is another VGAM206 host target gene. LOC219287 BINDING SITE is HOST TARGET binding site found in the 3′ untranslated region of mRNA encoded by LOC219287, corresponding to a HOST TARGET binding site such as BINDING SITE I, BINDING SITE II or BINDING SITE III. Table 2 illustrates the complementarity of the nucleotide sequences of LOC219287 BINDING SITE, designated SEQ ID:3609, to the nucleotide sequence of VGAM206 RNA, herein designated VGAM RNA, also designated SEQ ID:541.

Another function of VGAM206 is therefore inhibition of LOC219287 (Accession XM_{—}167994). Accordingly, utilities of VGAM206 include diagnosis, prevention and treatment of diseases and clinical conditions associated with LOC219287. LOC221979 (Accession XM_{—}166540) is another VGAM206 host target gene. LOC221979 BINDING SITE is HOST TARGET binding site found in the 5′ untranslated region of mRNA encoded by LOC221979, corresponding to a HOST TARGET binding site such as BINDING SITE I, BINDING SITE II or BINDING SITE III. Table 2 illustrates the complementarity of the nucleotide sequences of LOC221979 BINDING SITE, designated SEQ ID:3571, to the nucleotide sequence of VGAM206 RNA, herein designated VGAM RNA, also designated SEQ ID:541.

Another function of VGAM206 is therefore inhibition of LOC221979 (Accession XM_{—}166540). Accordingly, utilities of VGAM206 include diagnosis, prevention and treatment of diseases and clinical conditions associated with LOC221979. LOC255328 (Accession XM_{—}172920) is another VGAM206 host target gene. LOC255328 BINDING SITE is HOST TARGET binding site found in the 3′ untranslated region of mRNA encoded by LOC255328, corresponding to a HOST TARGET binding site such as BINDING SITE I, BINDING SITE II or BINDING SITE III. Table 2 illustrates the complementarity of the nucleotide sequences of LOC255328 BINDING SITE, designated SEQ ID:3710, to the nucleotide sequence of VGAM206 RNA, herein designated VGAM RNA, also designated SEQ ID:541.

Another function of VGAM206 is therefore inhibition of LOC255328 (Accession XM_{—}172920). Accordingly, utilities of VGAM206 include diagnosis, prevention and treatment of diseases and clinical conditions associated with LOC255328. LOC257115 (Accession XM_{—}172898) is another VGAM206 host target gene. LOC257115 BINDING SITE is HOST TARGET binding site found in the 3′ untranslated region of mRNA encoded by LOC257115, corresponding to a HOST TARGET binding site such as BINDING SITE I, BINDING SITE II or BINDING SITE III. Table 2 illustrates the complementarity of the nucleotide sequences of LOC257115 BINDING SITE, designated SEQ ID:3709, to the nucleotide sequence of VGAM206 RNA, herein designated VGAM RNA, also designated SEQ ID:541.

Another function of VGAM206 is therefore inhibition of LOC257115 (Accession XM_{—}172898). Accordingly, utilities of VGAM206 include diagnosis, prevention and treatment of diseases and clinical conditions associated with LOC257115. LOC51145 (Accession NM_{—}016158) is another VGAM206 host target gene. LOC51145 BINDING SITE is HOST TARGET binding site found in the 3′ untranslated region of mRNA encoded by LOC51145, corresponding to a HOST TARGET binding site such as BINDING SITE I, BINDING SITE II or BINDING SITE III. Table 2 illustrates the complementarity of the nucleotide sequences of LOC51145 BINDING SITE, designated SEQ ID:1666, to the nucleotide sequence of VGAM206 RNA, herein designated VGAM RNA, also designated SEQ ID:541.

Another function of VGAM206 is therefore inhibition of LOC51145 (Accession NM_{—}016158). Accordingly, utilities of VGAM206 include diagnosis, prevention and treatment of diseases and clinical conditions associated with LOC51145. FIG. 1 further provides a conceptual description of a novel bioinformatically detected viral gene of the present invention, referred to here as Viral Genomic Address Messenger 207 (VGAM207) viral gene, which modulates expression of respective host target genes thereof, the function and utility of which host target genes is known in the art.

VGAM207 is a novel bioinformatically detected regulatory, non protein coding, viral micro RNA (miRNA) gene. The method by which VGAM207 was detected is described hereinabove with reference to FIGS. 1-8.

VGAM207 gene, herein designated VGAM GENE, is a viral gene contained in the genome of Vaccinia Virus. VGAM207 host target gene, herein designated VGAM HOST TARGET GENE, is a human gene contained in the human genome.

VGAM207 gene encodes a VGAM207 precursor RNA, herein designated VGAM PRECURSOR RNA. Similar to other miRNA genes, and unlike most ordinary genes, VGAM207 precursor RNA does not encode a protein. A nucleotide sequence identical or highly similar to the nucleotide sequence of VGAM207 precursor RNA is designated SEQ ID:193, and is provided hereinbelow with reference to the sequence listing part. Nucleotide sequence SEQ ID:193 is located at position 109549 relative to the genome of Vaccinia Virus.

VGAM207 precursor RNA folds onto itself, forming VGAM207 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM207 folded precursor RNA into VGAM207 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM207 RNA is designated SEQ ID:542, and is provided hereinbelow with reference to the sequence listing part.

VGAM207 host target gene, herein designated VGAM HOST TARGET GENE, encodes a corresponding messenger RNA, VGAM207 host target RNA, herein designated VGAM HOST TARGET RNA. VGAM207 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM207 RNA, herein designated VGAM RNA, binds complementarily to one or more host target binding sites located in untranslated regions of VGAM207 host target RNA, herein designated VGAM HOST TARGET RNA. This complementary binding is due to the fact that the nucleotide sequence of VGAM207 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM207 RNA, herein designated VGAM RNA, may have a different number of host target binding sites in untranslated regions of a VGAM207 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM207 RNA, herein designated VGAM RNA, to host target binding sites on VGAM207 host target RNA, herein designated VGAM HOST TARGET RNA, such as BINDING SITE I, BINDING SITE II and BINDING SITE III, inhibits translation of VGAM207 host target RNA into VGAM207 host target protein, herein designated VGAM HOST TARGET PROTEIN. VGAM host target protein is therefore outlined by a broken line.

It is appreciated that VGAM207 host target gene, herein designated VGAM HOST TARGET GENE, in fact represents a plurality of VGAM207 host target genes. The mRNA of each one of this plurality of VGAM207 host target genes comprises one or more host target binding sites, each having a nucleotide sequence which is at least partly complementary to VGAM207 RNA, herein designated VGAM RNA, and which when bound by VGAM207 RNA causes inhibition of translation of respective one or more VGAM207 host target proteins.

It is further appreciated by one skilled in the art that the mode of translational inhibition illustrated by FIG. 1 with specific reference to translational inhibition exerted by VGAM207 gene, herein designated VGAM GENE, on one or more VGAM207 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM207 is inhibition of expression of host target genes, as part of a novel viral mechanism of attacking a host. Accordingly, utilities of VGAM207 include diagnosis, prevention and treatment of viral infection by Vaccinia Virus. Specific functions, and accordingly utilities, of VGAM207 correlate with, and may be deduced from, the identity of the host target genes which VGAM207 binds and inhibits, and the function of these host target genes, as elaborated hereinbelow.

Nucleotide sequences of the VGAM207 precursor RNA, herein designated VGAM PRECURSOR RNA, and of the ‘diced’ VGAM207 RNA, herein designated VGAM RNA, and a schematic representation of the secondary folding of VGAM207 folded precursor RNA, herein designated VGAM FOLDED PRECURSOR RNA, of VGAM207 are further described hereinbelow with reference to Table 1.

Nucleotide sequences of host target binding sites, such as BINDING SITE-I, BINDING SITE-II and BINDING SITE-III of FIG. 1, found on VGAM207 host target RNA, and schematic representation of the complementarity of each of these host target binding sites to VGAM207 RNA, herein designated VGAM RNA, are described hereinbelow with reference to Table 2.

As mentioned hereinabove with reference to FIG. 1, a function of VGAM207 gene, herein designated VGAM is inhibition of expression of VGAM207 target genes. It is appreciated that specific functions, and accordingly utilities, of VGAM207 correlate with, and may be deduced from, the identity of the target genes which VGAM207 binds and inhibits, and the function of these target genes, as elaborated hereinbelow.

B-cell CLL/lymphoma 11A (zinc finger protein) (BCL11A, Accession NM_{—}022893) is a VGAM207 host target gene. BCL11A BINDING SITE is HOST TARGET binding site found in the 3′ untranslated region of mRNA encoded by BCL11A, corresponding to a HOST TARGET binding site such as BINDING SITE I, BINDING SITE II or BINDING SITE III. Table 2 illustrates the complementarity of the nucleotide sequences of BCL11A BINDING SITE, designated SEQ ID:2018, to the nucleotide sequence of VGAM207 RNA, herein designated VGAM RNA, also designated SEQ ID:542.

A function of VGAM207 is therefore inhibition of B-cell CLL/lymphoma 11A (zinc finger protein) (BCL11A, Accession NM_{—}022893), a gene which acts as a transcriptional repressor. Accordingly, utilities of VGAM207 include diagnosis, prevention and treatment of diseases and clinical conditions associated with BCL11A. The function of BCL11A has been established by previous studies. By screening a fetal brain cDNA library with mouse Evi9 as probe, Saiki et al. (2000) isolated a cDNA encoding EVI9, also termed BCL11A, and a shorter splice variant, EVI9C. Sequence analysis predicted that the 797-amino acid BCL11A protein, which is 99% identical to the mouse protein apart from an additional 35 N-terminal residues, contains 3 C2H2-type zinc finger motifs, a proline-rich region, and an acidic domain. Northern blot analysis revealed highest expression in brain, spleen, and testis. RT-PCR analysis detected expression in most hematopoietic cells but downregulation during monocytic differentiation. Satterwhite et al. (2001) reported the recurrent involvement and deregulated expression of BCL11A in 4 cases of B-cell malignancy with the translocation t(2;14)(p13;q32.3). They noted that this translocation is a rare cytogenetic abnormality in the clinically aggressive subset of B-cell chronic lymphocytic leukemia (OMIM Ref. No. 151400)/immunocytoma. FISH analysis showed colocalization of BCL11A and REL (OMIM Ref. No. 164910) in B-cell non-Hodgkin lymphoma (OMIM Ref. No. 605027). Satterwhite et al. (2001) also identified a BCL11A homolog, BCL11B (OMIM Ref. No. 606558). Comparative genomic hybridization studies showed gains in chromosome region 2p as the most common imbalance in classical Hodgkin lymphoma. The minimal region of gain contained 2 candidate oncogenes, REL and BCL11A. Martin-Subero et al. (2002) examined the involvement of REL and BCL11A loci in 44 primary cases of classic Hodgkin lymphoma by combined immunophenotyping and interphase cytogenetics. A median 2p13 copy number above the tetraploid range was detected in 24 (55%) cases. One case displayed selective amplification of the REL locus not affecting BCL11A. Two other cases showed evidence of breakpoints in the region spanned by the REL probe. These data indicated that REL rather than BCL11A may be the target of the 2p13 alterations in classic Hodgkin lymphoma.

Full details of the abovementioned studies are described in the following publications, the disclosure of which are hereby incorporated by reference:
Satterwhite, E.; Sonoki, T.; Willis, T. G.; Harder, L.; Nowak, R.; Arriola, E. L.; Liu, H.; Price, H. P.; Gesk, S.; Steinemann, D.; Schlegelberger, B.; Oscier, D. G.; Siebert, R.; Tucker, P. W.; Dyer, M. J. S.: The BCL11 gene family: involvement of BCL11A in lymphoid malignancies. Blood 98: 3413-3420, 2001.; and
Martin-Subero, J. I.; Gesk, S.; Harder, L.; Sonoki, T.; Tucker, P. W.; Schlegelberger, B.; Grote, W.; Novo, F. J.; Calasanz, M. J.; Hansmann, M. L.; Dyer, M. J. S.; Siebert, R.: Recurr.

Further studies establishing the function and utilities of BCL11A are found in John Hopkins OMIM database record ID 606557, and in sited publications numbered 2687-1277 listed in the bibliography section hereinbelow, which are also hereby incorporated by reference. Chromosome 8 Open Reading Frame 1 (C8orf1, Accession NM_{—}004337) is another VGAM207 host target gene. C8orf1 BINDING SITE is HOST TARGET binding site found in the 3′ untranslated region of mRNA encoded by C8orf1, corresponding to a HOST TARGET binding site such as BINDING SITE I, BINDING SITE II or BINDING SITE III. Table 2 illustrates the complementarity of the nucleotide sequences of C8orf1 BINDING SITE, designated SEQ ID: 1099, to the nucleotide sequence of VGAM207 RNA, herein designated VGAM RNA, also designated SEQ ID:542.

Another function of VGAM207 is therefore inhibition of Chromosome 8 Open Reading Frame 1 (C8orf1, Accession NM_{—}004337). Accordingly, utilities of VGAM207 include diagnosis, prevention and treatment of diseases and clinical conditions associated with C8orf1. Karyopherin Alpha 1 (importin alpha 5) (KPNA1, Accession XM_{—}087256) is another VGAM207 host target gene. KPNA1 BINDING SITE is HOST TARGET binding site found in the 3′ untranslated region of mRNA encoded by KPNA1, corresponding to a HOST TARGET binding site such as BINDING SITE I, BINDING SITE II or BINDING SITE III. Table 2 illustrates the complementarity of the nucleotide sequences of KPNA1 BINDING SITE, designated SEQ ID:3159, to the nucleotide sequence of VGAM207 RNA, herein designated VGAM RNA, also designated SEQ ID:542.

Another function of VGAM207 is therefore inhibition of Karyopherin Alpha 1 (importin alpha 5) (KPNA1, Accession XM_{—}087256), a gene which promotes docking of import substrates to the nuclear pore complex. Accordingly, utilities of VGAM207 include diagnosis, prevention and treatment of diseases and clinical conditions associated with KPNA1. The function of KPNA1 has been established by previous studies. Cortes et al. (1994) used the 2-hybrid protein interaction systems to isolate a protein that specifically interacts with RAG1 (OMIM Ref. No. 179615). The genes RAG1 and RAG2 (OMIM Ref. No. 179616) are able to activate V(D)J recombination when transfected into fibroblasts. Further, knockout mice for these 2 loci lack B and T cells. Several other ubiquitously expressed proteins are thought to be recruited in the recombination process. Among these are the genes affected in severe combined immune deficiency (e.g., OMIM Ref. No. also 600899) and genes involved in ds-DNA break repair. The human cDNA identified by Cortes et al. (1994) encodes a 489-amino acid polypeptide that shows striking similarity to the yeast SRP1 protein, a mutant allele which can suppress a mutation of RNA polymerase I. The authors obtained human and mouse cDNA sequences which are 98% identical as proteins. When RAG1 and human SRP1 were cotransfected into 293T cells a stable complex of the 2 was observed. The authors speculated that because SRP1 appears to be bound to the nuclear envelope, the interaction with RAG1 may serve to localize that protein to the envelope as well. Conti et al. (1998) reported the crystal structure of a 50-kD fragment of the 60-kD yeast karyopherin alpha, in the absence and presence of a monopartite nuclear localization signal (NLS) peptide at 2.2-angstrom and 2.8-angstrom resolution, respectively. The structure showed a tandem array of 10 armadillo repeats, organized in a right-handed superhelix of helices. Binding of the NLS peptide occurred at 2 sites within a helical surface groove. The structure reveals the determinants of NLS specificity and suggested a model for the recognition of bipartite NLSs. By fluorescence in situ hybridization, Ayala-Madrigal et al. (2000) mapped the human KPNA1 gene to chromosome 3q21.

Full details of the abovementioned studies are described in the following publications, the disclosure of which are hereby incorporated by reference:
Ayala-Madrigal, M. L.; Doerr, S.; Ramirez-Duenas, M. L.; Hansmann, I.: Assignment of karyopherin alpha 1 (KPNA1) to human chromosome band 3q21 by in situ hybridization. Cytogenet. Cell Genet. 90: 58-59, 2000.; and
Conti, E.; Uy, M.; Leighton, L.; Blobel, G.; Kuriyan, J.: Crystallographic analysis of the recognition of a nuclear localization signal by the nuclear import factor karyopherin alpha.

Further studies establishing the function and utilities of KPNA1 are found in John Hopkins OMIM database record ID 600686, and in sited publications numbered 2247-2249 listed in the bibliography section hereinbelow, which are also hereby incorporated by reference. Neurexin 1 (NRXN1, Accession NM_{—}138735) is another VGAM207 host target gene. NRXN1 BINDING SITE1 and NRXN1 BINDING SITE2 are HOST TARGET binding sites found in untranslated regions of mRNA encoded by NRXN1, corresponding to HOST TARGET binding sites such as BINDING SITE I, BINDING SITE II or BINDING SITE III. Table 2 illustrates the complementarity of the nucleotide sequences of NRXN1 BINDING SITE1 and NRXN1 BINDING SITE2, designated SEQ ID:2457 and SEQ ID:1158 respectively, to the nucleotide sequence of VGAM207 RNA, herein designated VGAM RNA, also designated SEQ ID:542.

Another function of VGAM207 is therefore inhibition of Neurexin 1 (NRXN1, Accession NM_{—}138735), a gene which may be involved in cell recognition, cell adhesion, and mediate intracellular signaling. Accordingly, utilities of VGAM207 include diagnosis, prevention and treatment of diseases and clinical conditions associated with NRXN1. The function of NRXN1 has been established by previous studies. Neurexins are polymorphic cell surface proteins that are expressed in neurons. They were discovered by Ushkaryov et al. (1992) in the course of cloning the presynaptic receptor for alpha-latrotoxin. Three neurexin genes, designated 1 (NRXN1), 2 (NRXN2; 600566), and 3 (NRXN3; 600567), were identified in a rat brain cDNA library by Ushkaryov et al. (1992). Ichtchenko et al. (1995) observed that each neurexin gene has 2 independent promoters which generate 2 classes of mRNAs: the longer mRNAs encode alpha-neurexins and the shorter mRNAs encode beta-neurexins. Thus, 6 principal neurexin isoforms, called neurexins I-alpha to III-beta, result, of which neurexin I-alpha corresponds to the high molecular weight component of the alpha-latrotoxin receptor. Ushkaryov et al. (1992) showed that rat neurexins are expressed at significant levels only in brain. Ullrich et al. (1995) found that the 6 rat neurexin isoforms are coexpressed in neurons and are distributed differentially in various brain regions. Neurexins display a remarkable evolutionarily conserved pattern of extensive alternative splicing. As a result, the total number of neurexins in brain probably exceeds 2,000 (Ullrich et al., 1995). Neurexins contain epidermal growth factor-like sequences and domains homologous to the G domain repeats of laminin A (LAMA; 150320), indicating a function in cell-cell interactions. Animal model experiments lend further support to the function of NRXN1. Alpha-latrotoxin is a potent neurotoxin from black widow spider venom that binds to presynaptic receptors and causes massive neurotransmitter release. In rat, 2 alpha-latrotoxin receptors have been identified: neurexin I-alpha, which binds the toxin in a calcium-dependent manner, and CIRL/latrophilin, which binds in a calcium-independent manner. Geppert et al. (1998) isolated the mouse neurexin I-alpha gene and found that it contains a large first exon of more than 1.5 kb that extends to the first site of alternative splicing in the coding region. To evaluate the importance of neurexin I-alpha in alpha-latrotoxin action, Geppert et al. (1998) generated mice carrying a deletion of the first exon of the neurexin I-alpha gene. Homozygous mutant mice lacked neurexin I-alpha, although the levels of neurexin 1-beta were unaffected. The mutant mice were viable and fertile, and were indistinguishable in appearance from wildtype animals. The only abnormality observed was that female knockout mice were less able to attend to litters, leading to the death of more pups independent of pup genotype. Geppert et al. (1998) found that alpha-latrotoxin binding to brain membranes from mutant mice was decreased by almost 50% compared with wildtype membranes. In cultured hippocampal neurons from mutant mice, the toxin was still capable of activating neurotransmission. However, measurements of glutamate release from synaptosomes indicated a major decrease in the amount of release triggered by alpha-latrotoxin in the presence of calcium. The authors concluded that neurexin I-alpha is not essential for alpha-latrotoxin action but contributes to toxin action when calcium is present. They suggested that the action of alpha-latrotoxin may be mediated by independent parallel pathways.

It is appreciated that the abovementioned animal model for NRXN1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Geppert, M.; Khvotchev, M.; Krasnoperov, V.; Goda, Y.; Missler, M.; Hammer, R. E.; Ichtchenko, K.; Petrenko, A. G.; Sudhof, T. C.: Neurexin I-alpha is a major alpha-latrotoxin receptor that cooperates in alpha-latrotoxin action. J. Biol. Chem. 273: 1705-1710, 1998.; and
Ushkaryov, Y. A.; Petrenko, A. G.; Geppert, M.; Sudhof, T. C.: Neurexins: synaptic cell surface proteins related to the alpha-latrotoxin receptor and laminin. Science 257: 50-56, 199.

Further studies establishing the function and utilities of NRXN1 are found in John Hopkins OMIM database record ID 600565, and in sited publications numbered 1859-1863, 156 and 2146-2149 listed in the bibliography section hereinbelow, which are also hereby incorporated by reference. Sorting Nexin 5 (SNX5, Accession NM_{—}014426) is another VGAM207 host target gene. SNX5 BINDING SITE is HOST TARGET binding site found in the 3′ untranslated region of mRNA encoded by SNX5, corresponding to a HOST TARGET binding site such as BINDING SITE I, BINDING SITE II or BINDING SITE III. Table 2 illustrates the complementarity of the nucleotide sequences of SNX5 BINDING SITE, designated SEQ ID:1500, to the nucleotide sequence of VGAM207 RNA, herein designated VGAM RNA, also designated SEQ ID:542.

Another function of VGAM207 is therefore inhibition of Sorting Nexin 5 (SNX5, Accession NM_{—}014426). Accordingly, utilities of VGAM207 include diagnosis, prevention and treatment of diseases and clinical conditions associated with SNX5. BCL2/adenovirus E1B 19 kDa Interacting Protein 2 (BNIP2, Accession XM_{—}039703) is another VGAM207 host target gene. BNIP2 BINDING SITE is HOST TARGET binding site found in the 3′ untranslated region of mRNA encoded by BNIP2, corresponding to a HOST TARGET binding site such as BINDING SITE I, BINDING SITE II or BINDING SITE III. Table 2 illustrates the complementarity of the nucleotide sequences of BNIP2 BINDING SITE, designated SEQ ID:2762, to the nucleotide sequence of VGAM207 RNA, herein designated VGAM RNA, also designated SEQ ID:542.

Another function of VGAM207 is therefore inhibition of BCL2/adenovirus E1B 19 kDa Interacting Protein 2 (BNIP2, Accession XM_{—}039703). Accordingly, utilities of VGAM207 include diagnosis, prevention and treatment of diseases and clinical conditions associated with BNIP2. DRIL2 (Accession NM_{—}006465) is another VGAM207 host target gene. DRIL2 BINDING SITE is HOST TARGET binding site found in the 3′ untranslated region of mRNA encoded by DRIL2, corresponding to a HOST TARGET binding site such as BINDING SITE I, BINDING SITE II or BINDING SITE III. Table 2 illustrates the complementarity of the nucleotide sequences of DRIL2 BINDING SITE, designated SEQ ID:1305, to the nucleotide sequence of VGAM207 RNA, herein designated VGAM RNA, also designated SEQ ID:542.

Another function of VGAM207 is therefore inhibition of DRIL2 (Accession NM_{—}006465). Accordingly, utilities of VGAM207 include diagnosis, prevention and treatment of diseases and clinical conditions associated with DRIL2. DVS27 (Accession NM_{—}033439) is another VGAM207 host target gene. DVS27 BINDING SITE is HOST TARGET binding site found in the 3′ untranslated region of mRNA encoded by DVS27, corresponding to a HOST TARGET binding site such as BINDING SITE I, BINDING SITE II or BINDING SITE III. Table 2 illustrates the complementarity of the nucleotide sequences of DVS27 BINDING SITE, designated SEQ ID:2335, to the nucleotide sequence of VGAM207 RNA, herein designated VGAM RNA, also designated SEQ ID:542.

Another function of VGAM207 is therefore inhibition of DVS27 (Accession NM_{—}033439). Accordingly, utilities of VGAM207 include diagnosis, prevention and treatment of diseases and clinical conditions associated with DVS27. FLJ12960 (Accession NM_{—}024638) is another VGAM207 host target gene. FLJ12960 BINDING SITE is HOST TARGET binding site found in the 3′ untranslated region of mRNA encoded by FLJ12960, corresponding to a HOST TARGET binding site such as BINDING SITE I, BINDING SITE II or BINDING SITE III. Table 2 illustrates the complementarity of the nucleotide sequences of FLJ12960 BINDING SITE, designated SEQ ID:2074, to the nucleotide sequence of VGAM207 RNA, herein designated VGAM RNA, also designated SEQ ID:542.

Another function of VGAM207 is therefore inhibition of FLJ12960 (Accession NM_{—}024638). Accordingly, utilities of VGAM207 include diagnosis, prevention and treatment of diseases and clinical conditions associated with FLJ12960. FLJ20793 (Accession XM_{—}166296) is another VGAM207 host target gene. FLJ20793 BINDING SITE is HOST TARGET binding site found in the 3′ untranslated region of mRNA encoded by FLJ20793, corresponding to a HOST TARGET binding site such as BINDING SITE I, BINDING SITE II or BINDING SITE III. Table 2 illustrates the complementarity of the nucleotide sequences of FLJ20793 BINDING SITE, designated SEQ ID:3542, to the nucleotide sequence of VGAM207 RNA, herein designated VGAM RNA, also designated SEQ ID:542.

Another function of VGAM207 is therefore inhibition of FLJ20793 (Accession XM_{—}166296). Accordingly, utilities of VGAM207 include diagnosis, prevention and treatment of diseases and clinical conditions associated with FLJ20793. KIAA0040 (Accession NM_{—}014656) is another VGAM207 host target gene. KIAA0040 BINDING SITE is HOST TARGET binding site found in the 3′ untranslated region of mRNA encoded by KIAA0040, corresponding to a HOST TARGET binding site such as BINDING SITE I, BINDING SITE II or BINDING SITE III. Table 2 illustrates the complementarity of the nucleotide sequences of KIAA0040 BINDING SITE, designated SEQ ID:1515, to the nucleotide sequence of VGAM207 RNA, herein designated VGAM RNA, also designated SEQ ID:542.

Another function of VGAM207 is therefore inhibition of KIAA0040 (Accession NM_{—}014656). Accordingly, utilities of VGAM207 include diagnosis, prevention and treatment of diseases and clinical conditions associated with KIAA0040. KIAA1349 (Accession XM_{—}047617) is another VGAM207 host target gene. KIAA1349 BINDING SITE is HOST TARGET binding site found in the 3′ untranslated region of mRNA encoded by KIAA1349, corresponding to a HOST TARGET binding site such as BINDING SITE I, BINDING SITE II or BINDING SITE III. Table 2 illustrates the complementarity of the nucleotide sequences of KIAA1349 BINDING SITE, designated SEQ ID:2893, to the nucleotide sequence of VGAM207 RNA, herein designated VGAM RNA, also designated SEQ ID:542.

Another function of VGAM207 is therefore inhibition of KIAA1349 (Accession XM_{—}047617). Accordingly, utilities of VGAM207 include diagnosis, prevention and treatment of diseases and clinical conditions associated with KIAA1349. KIAA1373 (Accession XM_{—}048195) is another VGAM207 host target gene. KIAA1373 BINDING SITE is HOST TARGET binding site found in the 3′ untranslated region of mRNA encoded by KIAA1373, corresponding to a HOST TARGET binding site such as BINDING SITE I, BINDING SITE II or BINDING SITE III. Table 2 illustrates the complementarity of the nucleotide sequences of KIAA1373 BINDING SITE, designated SEQ ID:2903, to the nucleotide sequence of VGAM207 RNA, herein designated VGAM RNA, also designated SEQ ID:542.

Another function of VGAM207 is therefore inhibition of KIAA1373 (Accession XM_{—}048195). Accordingly, utilities of VGAM207 include diagnosis, prevention and treatment of diseases and clinical conditions associated with KIAA1373. SCDGF-B (Accession NM_{—}025208) is another VGAM207 host target gene. SCDGF-B BINDING SITE1 and SCDGF-B BINDING SITE2 are HOST TARGET binding sites found in untranslated regions of mRNA encoded by SCDGF-B, corresponding to HOST TARGET binding sites such as BINDING SITE I, BINDING SITE II or BINDING SITE III. Table 2 illustrates the complementarity of the nucleotide sequences of SCDGF-B BINDING SITE1 and SCDGF-B BINDING SITE2, designated SEQ ID:2147 and SEQ ID:2314 respectively, to the nucleotide sequence of VGAM207 RNA, herein designated VGAM RNA, also designated SEQ ID:542.

Another function of VGAM207 is therefore inhibition of SCDGF-B (Accession NM_{—}025208). Accordingly, utilities of VGAM207 include diagnosis, prevention and treatment of diseases and clinical conditions associated with SCDGF-B. LOC157729 (Accession XM_{—}088374) is another VGAM207 host target gene. LOC157729 BINDING SITE is HOST TARGET binding site found in the 3′ untranslated region of mRNA encoded by LOC157729, corresponding to a HOST TARGET binding site such as BINDING SITE I, BINDING SITE II or BINDING SITE III. Table 2 illustrates the complementarity of the nucleotide sequences of LOC157729 BINDING SITE, designated SEQ ID:3205, to the nucleotide sequence of VGAM207 RNA, herein designated VGAM RNA, also designated SEQ ID:542.

Another function of VGAM207 is therefore inhibition of LOC157729 (Accession XM_{—}088374). Accordingly, utilities of VGAM207 include diagnosis, prevention and treatment of diseases and clinical conditions associated with LOC157729. LOC200339 (Accession XM_{—}117226) is another VGAM207 host target gene. LOC200339 BINDING SITE is HOST TARGET binding site found in the 3′ untranslated region of mRNA encoded by LOC200339, corresponding to a HOST TARGET binding site such as BINDING SITE I, BINDING SITE II or BINDING SITE III. Table 2 illustrates the complementarity of the nucleotide sequences of LOC200339 BINDING SITE, designated SEQ ID:3475, to the nucleotide sequence of VGAM207 RNA, herein designated VGAM RNA, also designated SEQ ID:542.

Another function of VGAM207 is therefore inhibition of LOC200339 (Accession XM_{—}117226). Accordingly, utilities of VGAM207 include diagnosis, prevention and treatment of diseases and clinical conditions associated with LOC200339. LOC221300 (Accession XM_{—}166322) is another VGAM207 host target gene. LOC221300 BINDING SITE is HOST TARGET binding site found in the 3′ untranslated region of mRNA encoded by LOC221300, corresponding to a HOST TARGET binding site such as BINDING SITE I, BINDING SITE II or BINDING SITE III. Table 2 illustrates the complementarity of the nucleotide sequences of LOC221300 BINDING SITE, designated SEQ ID:3545, to the nucleotide sequence of VGAM207 RNA, herein designated VGAM RNA, also designated SEQ ID:542.

Another function of VGAM207 is therefore inhibition of LOC221300 (Accession XM_{—}166322). Accordingly, utilities of VGAM207 include diagnosis, prevention and treatment of diseases and clinical conditions associated with LOC221300. LOC257235 (Accession XM_{—}173124) is another VGAM207 host target gene. LOC257235 BINDING SITE is HOST TARGET binding site found in the 5′ untranslated region of mRNA encoded by LOC257235, corresponding to a HOST TARGET binding site such as BINDING SITE I, BINDING SITE II or BINDING SITE III. Table 2 illustrates the complementarity of the nucleotide sequences of LOC257235 BINDING SITE, designated SEQ ID:3728, to the nucleotide sequence of VGAM207 RNA, herein designated VGAM RNA, also designated SEQ ID:542.

Another function of VGAM207 is therefore inhibition of LOC257235 (Accession XM_{—}173124). Accordingly, utilities of VGAM207 include diagnosis, prevention and treatment of diseases and clinical conditions associated with LOC257235. LOC93333 (Accession XM_{—}050624) is another VGAM207 host target gene. LOC93333 BINDING SITE is HOST TARGET binding site found in the 5′ untranslated region of mRNA encoded by LOC93333, corresponding to a HOST TARGET binding site such as BINDING SITE I, BINDING SITE II or BINDING SITE III. Table 2 illustrates the complementarity of the nucleotide sequences of LOC93333 BINDING SITE, designated SEQ ID:2932, to the nucleotide sequence of VGAM207 RNA, herein designated VGAM RNA, also designated SEQ ID:542.

Another function of VGAM207 is therefore inhibition of LOC93333 (Accession XM_{—}050624). Accordingly, utilities of VGAM207 include diagnosis, prevention and treatment of diseases and clinical conditions associated with LOC93333. FIG. 1 further provides a conceptual description of a novel bioinformatically detected viral gene of the present invention, referred to here as Viral Genomic Address Messenger 208 (VGAM208) viral gene, which modulates expression of respective host target genes thereof, the function and utility of which host target genes is known in the art.

VGAM208 is a novel bioinformatically detected regulatory, non protein coding, viral micro RNA (miRNA) gene. The method by which VGAM208 was detected is described hereinabove with reference to FIGS. 1-8.

VGAM208 gene, herein designated VGAM GENE, is a viral gene contained in the genome of Vaccinia Virus. VGAM208 host target gene, herein designated VGAM HOST TARGET GENE, is a human gene contained in the human genome.

VGAM208 gene encodes a VGAM208 precursor RNA, herein designated VGAM PRECURSOR RNA. Similar to other miRNA genes, and unlike most ordinary genes, VGAM208 precursor RNA does not encode a protein. A nucleotide sequence identical or highly similar to the nucleotide sequence of VGAM208 precursor RNA is designated SEQ ID:194, and is provided hereinbelow with reference to the sequence listing part. Nucleotide sequence SEQ ID:194 is located at position 109656 relative to the genome of Vaccinia Virus.

VGAM208 precursor RNA folds onto itself, forming VGAM208 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM208 folded precursor RNA into VGAM208 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM208 RNA is designated SEQ ID:543, and is provided hereinbelow with reference to the sequence listing part.

VGAM208 host target gene, herein designated VGAM HOST TARGET GENE, encodes a corresponding messenger RNA, VGAM208 host target RNA, herein designated VGAM HOST TARGET RNA. VGAM208 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM208 RNA, herein designated VGAM RNA, binds complementarily to one or more host target binding sites located in untranslated regions of VGAM208 host target RNA, herein designated VGAM HOST TARGET RNA. This complementary binding is due to the fact that the nucleotide sequence of VGAM208 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM208 RNA, herein designated VGAM RNA, may have a different number of host target binding sites in untranslated regions of a VGAM208 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM208 RNA, herein designated VGAM RNA, to host target binding sites on VGAM208 host target RNA, herein designated VGAM HOST TARGET RNA, such as BINDING SITE I, BINDING SITE II and BINDING SITE III, inhibits translation of VGAM208 host target RNA into VGAM208 host target protein, herein designated VGAM HOST TARGET PROTEIN. VGAM host target protein is therefore outlined by a broken line.

It is appreciated that VGAM208 host target gene, herein designated VGAM HOST TARGET GENE, in fact represents a plurality of VGAM208 host target genes. The mRNA of each one of this plurality of VGAM208 host target genes comprises one or more host target binding sites, each having a nucleotide sequence which is at least partly complementary to VGAM208 RNA, herein designated VGAM RNA, and which when bound by VGAM208 RNA causes inhibition of translation of respective one or more VGAM208 host target proteins.

It is further appreciated by one skilled in the art that the mode of translational inhibition illustrated by FIG. 1 with specific reference to translational inhibition exerted by VGAM208 gene, herein designated VGAM GENE, on one or more VGAM208 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM208 is inhibition of expression of host target genes, as part of a novel viral mechanism of attacking a host. Accordingly, utilities of VGAM208 include diagnosis, prevention and treatment of viral infection by Vaccinia Virus. Specific functions, and accordingly utilities, of VGAM208 correlate with, and may be deduced from, the identity of the host target genes which VGAM208 binds and inhibits, and the function of these host target genes, as elaborated hereinbelow.

Nucleotide sequences of the VGAM208 precursor RNA, herein designated VGAM PRECURSOR RNA, and of the ‘diced’ VGAM208 RNA, herein designated VGAM RNA, and a schematic representation of the secondary folding of VGAM208 folded precursor RNA, herein designated VGAM FOLDED PRECURSOR RNA, of VGAM208 are further described hereinbelow with reference to Table 1.

Nucleotide sequences of host target binding sites, such as BINDING SITE-I, BINDING SITE-II and BINDING SITE-III of FIG. 1, found on VGAM208 host target RNA, and schematic representation of the complementarity of each of these host target binding sites to VGAM208 RNA, herein designated VGAM RNA, are described hereinbelow with reference to Table 2.

As mentioned hereinabove with reference to FIG. 1, a function of VGAM208 gene, herein designated VGAM is inhibition of expression of VGAM208 target genes. It is appreciated that specific functions, and accordingly utilities, of VGAM208 correlate with, and may be deduced from, the identity of the target genes which VGAM208 binds and inhibits, and the function of these target genes, as elaborated hereinbelow.

BH-protocadherin (brain-heart) (PCDH7, Accession NM_{—}002589) is a VGAM208 host target gene. PCDH7 BINDING SITE is HOST TARGET binding site found in the 3′ untranslated region of mRNA encoded by PCDH7, corresponding to a HOST TARGET binding site such as BINDING SITE I, BINDING SITE II or BINDING SITE III. Table 2 illustrates the complementarity of the nucleotide sequences of PCDH7 BINDING SITE, designated SEQ ID:938, to the nucleotide sequence of VGAM208 RNA, herein designated VGAM RNA, also designated SEQ ID:543.

A function of VGAM208 is therefore inhibition of BH-protocadherin (brain-heart) (PCDH7, Accession NM_{—}002589). Accordingly, utilities of VGAM208 include diagnosis, prevention and treatment of diseases and clinical conditions associated with PCDH7. TRC8 (Accession NM_{—}007218) is another VGAM208 host target gene. TRC8 BINDING SITE is HOST TARGET binding site found in the 3′ untranslated region of mRNA encoded by TRC8, corresponding to a HOST TARGET binding site such as BINDING SITE I, BINDING SITE II or BINDING SITE III. Table 2 illustrates the complementarity of the nucleotide sequences of TRC8 BINDING SITE, designated SEQ ID:1365, to the nucleotide sequence of VGAM208 RNA, herein designated VGAM RNA, also designated SEQ ID:543.

Another function of VGAM208 is therefore inhibition of TRC8 (Accession NM_{—}007218), a gene which Multiple membrane spanning protein; may be a signalling receptor and is presumed role as a tumor suppressor gene. Accordingly, utilities of VGAM208 include diagnosis, prevention and treatment of diseases and clinical conditions associated with TRC8. The function of TRC8 has been established by previous studies. Cohen et al. (1979) described a family in which a 3;8 chromosomal translocation, t(3;8)(p14.2;q24.1), was associated with classic features of hereditary renal cell carcinoma (OMIM Ref. No. 144700), i.e., autosomal dominant inheritance, early onset, and bilateral disease. Follow-up analysis found the occurrence of thyroid cancer in 2 translocation carriers with RCC (L1 et al., 1993). Although studies demonstrated that the 3p14.2 breakpoint interrupted the fragile histidine triad gene (FHIT; 601153) in its 5-prime noncoding region (Ohta et al., 1996), several reasons made it unlikely that FHIT is causally related to renal or other malignancies. Gemmill et al. (1998) showed that the 8q24.1 breakpoint region encodes a 664-amino acid multiple membrane-spanning protein, which they called TRC8, for ‘translocation in renal carcinoma, chromosome 8 gene.’ The gene showed similarity to the hereditary basal cell carcinoma/segment polarity gene, ‘patched.’ This similarity involved 2 regions of ‘patched,’ the putative sterol-sensing domain and the second extracellular loop that participates in the binding of sonic hedgehog (SHH; 600725). In the 3;8 translocation, TRC8 was found to be fused to FHIT and disrupted within the sterol-sensing domain. In contrast, the FHIT coding region was maintained and expressed. In a series of sporadic renal carcinomas, an acquired TRC8 mutation was identified. By analogy to patched, TRC8 might function as a signaling receptor, and other pathway members, to be defined, are mutation candidates in malignant diseases involving the kidney and thyroid. Gemmill et al. (2002) isolated the Drosophila homolog of TRC8 and studied its function by genetic manipulations and a yeast 2-hybrid screen. Human and Drosophila TRC8 proteins localize to the endoplasmic reticulum. Loss of either Drosophila Trc8 or Vhl resulted in an identical ventral midline defect. Direct interaction between Trc8 and Vhl in drosophila was confirmed by GST-pulldown and coimmunoprecipitation experiments. Gemmill et al. (2002) found that in Drosophila, overexpression of Trc8 inhibited growth consistent with its presumed role as a tumor suppressor gene. Human JAB1 (OMIM Ref. No. 604850) localization was dependent on VHL mutant status. Thus, the VHL, TRC8, and JAB1 proteins appear to be linked both physically and functionally, and all 3 may participate in the development of kidney cancer

Full details of the abovementioned studies are described in the following publications, the disclosure of which are hereby incorporated by reference:
Gemmill, R. M.; Bemis, L. T.; Lee, J. P.; Sozen, M. A.; Baron, A.; Zeng, C.; Erickson, P. F.; Hooper, J. E.; Drabkin, H. A.: The TRC8 hereditary kidney cancer gene suppresses growth and functions with VHL in a common pathway. Oncogene 21: 3507-3516, 2002.; and
Gemmill, R. M.; West, J. D.; Boldog, F.; Tanaka, N.; Robinson, L. J.; Smith, D. I.; Li, F.; Drabkin, H. A.: The hereditary renal cell carcinoma 3;8 translocation fuses FHIT to a patch.

Further studies establishing the function and utilities of TRC8 are found in John Hopkins OMIM database record ID 603046, and in sited publications numbered 301 and 3015-605 listed in the bibliography section hereinbelow, which are also hereby incorporated by reference. LOC145225 (Accession XM_{—}096741) is another VGAM208 host target gene. LOC145225 BINDING SITE is HOST TARGET binding site found in the 3′ untranslated region of mRNA encoded by LOC145225, corresponding to a HOST TARGET binding site such as BINDING SITE I, BINDING SITE II or BINDING SITE III. Table 2 illustrates the complementarity of the nucleotide sequences of LOC145225 BINDING SITE, designated SEQ ID:3275, to the nucleotide sequence of VGAM208 RNA, herein designated VGAM RNA, also designated SEQ ID:543.

Another function of VGAM208 is therefore inhibition of LOC145225 (Accession XM_{—}096741). Accordingly, utilities of VGAM208 include diagnosis, prevention and treatment of diseases and clinical conditions associated with LOC145225. FIG. 1 further provides a conceptual description of a novel bioinformatically detected viral gene of the present invention, referred to here as Viral Genomic Address Messenger 209 (VGAM209) viral gene, which modulates expression of respective host target genes thereof, the function and utility of which host target genes is known in the art.

VGAM209 is a novel bioinformatically detected regulatory, non protein coding, viral micro RNA (miRNA) gene. The method by which VGAM209 was detected is described hereinabove with reference to FIGS. 1-8.

VGAM209 gene, herein designated VGAM GENE, is a viral gene contained in the genome of Vaccinia Virus. VGAM209 host target gene, herein designated VGAM HOST TARGET GENE, is a human gene contained in the human genome.

VGAM209 gene encodes a VGAM209 precursor RNA, herein designated VGAM PRECURSOR RNA. Similar to other miRNA genes, and unlike most ordinary genes, VGAM209 precursor RNA does not encode a protein. A nucleotide sequence identical or highly similar to the nucleotide sequence of VGAM209 precursor RNA is designated SEQ ID:195, and is provided hereinbelow with reference to the sequence listing part. Nucleotide sequence SEQ ID:195 is located at position 109286 relative to the genome of Vaccinia Virus.

VGAM209 precursor RNA folds onto itself, forming VGAM209 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM209 folded precursor RNA into VGAM209 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 25%) nucleotide sequence of VGAM209 RNA is designated SEQ ID:544, and is provided hereinbelow with reference to the sequence listing part.

VGAM209 host target gene, herein designated VGAM HOST TARGET GENE, encodes a corresponding messenger RNA, VGAM209 host target RNA, herein designated VGAM HOST TARGET RNA. VGAM209 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM209 RNA, herein designated VGAM RNA, binds complementarily to one or more host target binding sites located in untranslated regions of VGAM209 host target RNA, herein designated VGAM HOST TARGET RNA. This complementary binding is due to the fact that the nucleotide sequence of VGAM209 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM209 RNA, herein designated VGAM RNA, may have a different number of host target binding sites in untranslated regions of a VGAM209 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM209 RNA, herein designated VGAM RNA, to host target binding sites on VGAM209 host target RNA, herein designated VGAM HOST TARGET RNA, such as BINDING SITE I, BINDING SITE II and BINDING SITE III, inhibits translation of VGAM209 host target RNA into VGAM209 host target protein, herein designated VGAM HOST TARGET PROTEIN. VGAM host target protein is therefore outlined by a broken line.

It is appreciated that VGAM209 host target gene, herein designated VGAM HOST TARGET GENE, in fact represents a plurality of VGAM209 host target genes. The mRNA of each one of this plurality of VGAM209 host target genes comprises one or more host target binding sites, each having a nucleotide sequence which is at least partly complementary to VGAM209 RNA, herein designated VGAM RNA, and which when bound by VGAM209 RNA causes inhibition of translation of respective one or more VGAM209 host target proteins.

It is further appreciated by one skilled in the art that the mode of translational inhibition illustrated by FIG. 1 with specific reference to translational inhibition exerted by VGAM209 gene, herein designated VGAM GENE, on one or more VGAM209 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM209 is inhibition of expression of host target genes, as part of a novel viral mechanism of attacking a host. Accordingly, utilities of VGAM209 include diagnosis, prevention and treatment of viral infection by Vaccinia Virus. Specific functions, and accordingly utilities, of VGAM209 correlate with, and may be deduced from, the identity of the host target genes which VGAM209 binds and inhibits, and the function of these host target genes, as elaborated hereinbelow.

Nucleotide sequences of the VGAM209 precursor RNA, herein designated VGAM PRECURSOR RNA, and of the ‘diced’ VGAM209 RNA, herein designated VGAM RNA, and a schematic representation of the secondary folding of VGAM209 folded precursor RNA, herein designated VGAM FOLDED PRECURSOR RNA, of VGAM209 are further described hereinbelow with reference to Table 1.

Nucleotide sequences of host target binding sites, such as BINDING SITE-I, BINDING SITE-II and BINDING SITE-III of FIG. 1, found on VGAM209 host target RNA, and schematic representation of the complementarity of each of these host target binding sites to VGAM209 RNA, herein designated VGAM RNA, are described hereinbelow with reference to Table 2.

As mentioned hereinabove with reference to FIG. 1, a function of VGAM209 gene, herein designated VGAM is inhibition of expression of VGAM209 target genes. It is appreciated that specific functions, and accordingly utilities, of VGAM209 correlate with, and may be deduced from, the identity of the target genes which VGAM209 binds and inhibits, and the function of these target genes, as elaborated hereinbelow.

Betaine-homocysteine Methyltransferase 2 (BHMT2, Accession NM_{—}017614) is a VGAM209 host target gene. BHMT2 BINDING SITE is HOST TARGET binding site found in the 3′ untranslated region of mRNA encoded by BHMT2, corresponding to a HOST TARGET binding site such as BINDING SITE I, BINDING SITE II or BINDING SITE III. Table 2 illustrates the complementarity of the nucleotide sequences of BHMT2 BINDING SITE, designated SEQ ID:1729, to the nucleotide sequence of VGAM209 RNA, herein designated VGAM RNA, also designated SEQ ID:544.

A function of VGAM209 is therefore inhibition of Betaine-homocysteine Methyltransferase 2 (BHMT2, Accession NM_{—}017614). Accordingly, utilities of VGAM209 include diagnosis, prevention and treatment of diseases and clinical conditions associated with BHMT2. Calnexin (CANX, Accession XM_{—}113469) is another VGAM209 host target gene. CANX BINDING SITE is HOST TARGET binding site found in the 3′ untranslated region of mRNA encoded by CANX, corresponding to a HOST TARGET binding site such as BINDING SITE I, BINDING SITE II or BINDING SITE III. Table 2 illustrates the complementarity of the nucleotide sequences of CANX BINDING SITE, designated SEQ ID:3409, to the nucleotide sequence of VGAM209 RNA, herein designated VGAM RNA, also designated SEQ ID:544.

Another function of VGAM209 is therefore inhibition of Calnexin (CANX, Accession XM_{—}113469), a gene which may function as a chaperone in the endoplasmic reticulum, involved in the secretion of proteins from the ER to the outer cellular membrane. Accordingly, utilities of VGAM209 include diagnosis, prevention and treatment of diseases and clinical conditions associated with CANX. The function of CANX has been established by previous studies. Calnexin is a 90-kilodalton integral membrane protein of the endoplasmic reticulum (ER). It exhibits high affinity for the binding of calcium ions, which was the means by which it was first identified. Calcium ions are known to play a central role in the regulation of cellular metabolism, including signal transduction events and the transport of proteins through the ER. Calnexin has been shown to be associated with several cell surface proteins during translocation through the ER and has been isolated as a complex with other ER proteins involved in calcium ion-dependent retention of proteins. It may function as a chaperone to regulate the transit of proteins through the ER. Tjoelker et al. (1994) isolated cDNA clones of the human, mouse, and rat calnexins. Comparisons of the sequences demonstrated a high level of conservation of sequence identity, suggesting that calnexin performs important cellular functions. Schwann cell-derived peripheral myelin protein-22 (PMP22; 601097), when mutated or overexpressed, causes heritable neuropathies with a ‘gain-of-function’ endoplasmic reticulum (ER) phenotype. PMP22 associates in a specific and transient manner with CANX in wildtype sciatic nerves. In the sciatic nerves of the Trembler (TrJ) mouse carrying the same mutation in the PMP22 gene that causes Charcot-Marie-Tooth disease (CMT) in the human, Dickson et al. (2002) found prolonged association of mutant PMP22 with CANX. In cultured cells expressing the TrJ mutant PMP22, CANX and PMP22 colocalized in large intracellular structures identified at the electron microscopy level as myelin-like figures, with CANX localization in the structures dependent on PMP22 glucosylation. Similar intracellular myelin-like figures were also present in Schwann cells of sciatic nerves from homozygous TrJ mice. Sequestration of CANX in intracellular myelin-like figures may be relevant to the pathogenesis of autosomal dominant CMT-related neuropathies.

Full details of the abovementioned studies are described in the following publications, the disclosure of which are hereby incorporated by reference:
Tjoelker, L. W.; Seyfried, C. E.; Eddy, R. L., Jr.; Byers, M. G.; Shows, T. B.; Calderon, J.; Schreiber, R. B.; Gray, P. W.: Human, mouse, and rat calnexin cDNA cloning: identification of potential calcium binding motifs and gene localization to human chromosome 5. Biochemistry 33: 3229-3236, 1994.; and
Dickson, K. M.; Bergeron, J. J. M.; Shames, I.; Colby, J.; Nguyen, D. T.; Chevet, E.; Thomas, D. Y.; Snipes, G. J.: Association of calnexin with mutant peripheral myelin protein-22 ex v.

Further studies establishing the function and utilities of CANX are found in John Hopkins OMIM database record ID 114217, and in sited publications numbered 899-902 listed in the bibliography section hereinbelow, which are also hereby incorporated by reference. Coatomer Protein Complex, Subunit Gamma 2 (COPG2, Accession XM_{—}088102) is another VGAM209 host target gene. COPG2 BINDING SITE is HOST TARGET binding site found in the 3′ untranslated region of mRNA encoded by COPG2, corresponding to a HOST TARGET binding site such as BINDING SITE I, BINDING SITE II or BINDING SITE III. Table 2 illustrates the complementarity of the nucleotide sequences of COPG2 BINDING SITE, designated SEQ ID:3194, to the nucleotide sequence of VGAM209 RNA, herein designated VGAM RNA, also designated SEQ ID:544.

Another function of VGAM209 is therefore inhibition of Coatomer Protein Complex, Subunit Gamma 2 (COPG2, Accession XM_{—}088102), a gene which mediates biosynthetic protein transport from the er, via the golgi up to the trans golgi network. Accordingly, utilities of VGAM209 include diagnosis, prevention and treatment of diseases and clinical conditions associated with COPG2. The function of COPG2 has been established by previous studies. Blagitko et al. (1999) sought to determine whether 7q32 harbors imprinted genes in addition to MEST (OMIM Ref. No. 601029), which is expressed in the paternal allele. They identified COPG2, a novel imprinted gene that overlaps with MEST and is transcribed from the paternally inherited allele in most fetal tissues, providing clear evidence that imprinting in 7q32 is regional. Expression was biallelic in fetal brain and liver and in adult peripheral blood. Biallelic expression in blood was supported by the demonstration of COPG2 transcripts in lymphoblastoid cell lines with maternal uniparental disomy 7. Absence of paternal COPG2 transcripts during embryonic development was thought to possibly contribute to Silver-Russell syndrome (OMIM Ref. No. 180860), which is associated with maternal uniparental disomy. However, mutation analysis in 42 patients with Silver-Russell syndrome and in 9 patients with primordial growth retardation detected only 1 maternally derived mutation in the COPG2 gene In an effort to understand how CDC42 (OMIM Ref. No. 116952) mediates cellular transformation, Wu et al. (2000) searched for new CDC42 targets. They identified COPG2 as a specific binding partner for activated CDC42. The binding of CDC42 to COPG2 is essential for a transforming signal distinct from those elicited by Ras (see 190020

Full details of the abovementioned studies are described in the following publications, the disclosure of which are hereby incorporated by reference:
Blagitko, N.; Schulz, U.; Schinzel, A. A.; Ropers, H.-H.; Kalscheuer, V. M.: Gamma-2-COP, a novel imprinted gene on chromosome 7q32, defines a new imprinting cluster in the human genome. Hum. Molec. Genet. 8: 2387-2396, 1999.; and
Wu, W. J.; Erickson, J. W.; Lin, R.; Cerione, R. A.: The gamma-subunit of the coatomer complex binds Cdc42 to mediate transformation. Nature 405: 800-804, 2000.

Further studies establishing the function and utilities of COPG2 are found in John Hopkins OMIM database record ID 604355, and in sited publications numbered 120 and 2811 listed in the bibliography section hereinbelow, which are also hereby incorporated by reference. Membrane-spanning 4-domains, Sub family A, Member 1 (MS4A1, Accession NM_{—}000139) is another VGAM209 host target gene. MS4A1 BINDING SITE is HOST TARGET binding site found in the 3′ untranslated region of mRNA encoded by MS4A1, corresponding to a HOST TARGET binding site such as BINDING SITE I, BINDING SITE II or BINDING SITE III. Table 2 illustrates the complementarity of the nucleotide sequences of MS4A1 BINDING SITE, designated SEQ ID:711, to the nucleotide sequence of VGAM209 RNA, herein designated VGAM RNA, also designated SEQ ID:544.

Another function of VGAM209 is therefore inhibition of Membrane-spanning 4-domains, Sub family A, Member 1 (MS4A1, Accession NM_{—}000139), a gene which may be involved in the regulation of b-cell activation and proliferation. Accordingly, utilities of VGAM209 include diagnosis, prevention and treatment of diseases and clinical conditions associated with MS4A1. The function of MS4A1 and its association with various diseases and clinical conditions, has been established by previous studies, as described hereinabove with reference to VGAM178. Sel-1 Suppressor of Lin-12-like (C. elegans) (SEL1L, Accession NM_{—}005065) is another VGAM209 host target gene. SEL1L BINDING SITE is HOST TARGET binding site found in the 3′ untranslated region of mRNA encoded by SEL1L, corresponding to a HOST TARGET binding site such as BINDING SITE I, BINDING SITE II or BINDING SITE III. Table 2 illustrates the complementarity of the nucleotide sequences of SEL1L BINDING SITE, designated SEQ ID:1180, to the nucleotide sequence of VGAM209 RNA, herein designated VGAM RNA, also designated SEQ ID:544.

Another function of VGAM209 is therefore inhibition of Sel-1 Suppressor of Lin-12-like (C. elegans) (SEL1L, Accession NM_{—}005065), a gene which may play a role in notch signaling (by similarity). Accordingly, utilities of VGAM209 include diagnosis, prevention and treatment of diseases and clinical conditions associated with SEL1L. The function of SEL1L and its association with various diseases and clinical conditions, has been established by previous studies, as described hereinabove with reference to VGAM99. Thiamin Pyrophosphokinase 1 (TPK1, Accession NM_{—}022445) is another VGAM209 host target gene. TPK1 BINDING SITE is HOST TARGET binding site found in the 3′ untranslated region of mRNA encoded by TPK1, corresponding to a HOST TARGET binding site such as BINDING SITE I, BINDING SITE II or BINDING SITE III. Table 2 illustrates the complementarity of the nucleotide sequences of TPK1 BINDING SITE, designated SEQ ID:1988, to the nucleotide sequence of VGAM209 RNA, herein designated VGAM RNA, also designated SEQ ID:544.

Another function of VGAM209 is therefore inhibition of Thiamin Pyrophosphokinase 1 (TPK1, Accession NM_{—}022445), a gene which catalyzes the conversion of thiamine, a form of vitamin B1, to thiamine pyrophosphate. Accordingly, utilities of VGAM209 include diagnosis, prevention and treatment of diseases and clinical conditions associated with TPK1. The function of TPK1 and its association with various diseases and clinical conditions, has been established by previous studies, as described hereinabove with reference to VGAM34. Chromosome Y Open Reading Frame 14 (CYorf14, Accession NM_{—}018542) is another VGAM209 host target gene. CYorf14 BINDING SITE is HOST TARGET binding site found in the 3′ untranslated region of mRNA encoded by CYorf14, corresponding to a HOST TARGET binding site such as BINDING SITE I, BINDING SITE II or BINDING SITE III. Table 2 illustrates the complementarity of the nucleotide sequences of CYorf14 BINDING SITE, designated SEQ ID: 1839, to the nucleotide sequence of VGAM209 RNA, herein designated VGAM RNA, also designated SEQ ID:544.

Another function of VGAM209 is therefore inhibition of Chromosome Y Open Reading Frame 14 (CYorf14, Accession NM_{—}018542). Accordingly, utilities of VGAM209 include diagnosis, prevention and treatment of diseases and clinical conditions associated with CYorf14. HSPC228 (Accession NM_{—}016485) is another VGAM209 host target gene. HSPC228 BINDING SITE is HOST TARGET binding site found in the 3′ untranslated region of mRNA encoded by HSPC228, corresponding to a HOST TARGET binding site such as BINDING SITE I, BINDING SITE II or BINDING SITE III. Table 2 illustrates the complementarity of the nucleotide sequences of HSPC228 BINDING SITE, designated SEQ ID: 1688, to the nucleotide sequence of VGAM209 RNA, herein designated VGAM RNA, also designated SEQ ID:544.

Another function of VGAM209 is therefore inhibition of HSPC228 (Accession NM_{—}016485). Accordingly, utilities of VGAM209 include diagnosis, prevention and treatment of diseases and clinical conditions associated with HSPC228. MGC1127 (Accession NM_{—}033549) is another VGAM209 host target gene. MGC1127 BINDING SITE is HOST TARGET binding site found in the 3′ untranslated region of mRNA encoded by MGC1127, corresponding to a HOST TARGET binding site such as BINDING SITE I, BINDING SITE II or BINDING SITE III. Table 2 illustrates the complementarity of the nucleotide sequences of MGC1127 BINDING SITE, designated SEQ ID:2336, to the nucleotide sequence of VGAM209 RNA, herein designated VGAM RNA, also designated SEQ ID:544.

Another function of VGAM209 is therefore inhibition of MGC1127 (Accession NM_{—}033549). Accordingly, utilities of VGAM209 include diagnosis, prevention and treatment of diseases and clinical conditions associated with MGC1127. Solute Carrier Family 16 (monocarboxylic acid transporters), Member 4 (SLC16A4, Accession NM_{—}004696) is another VGAM209 host target gene. SLC16A4 BINDING SITE is HOST TARGET binding site found in the 3′ untranslated region of mRNA encoded by SLC16A4, corresponding to a HOST TARGET binding site such as BINDING SITE I, BINDING SITE II or BINDING SITE III. Table 2 illustrates the complementarity of the nucleotide sequences of SLC16A4 BINDING SITE, designated SEQ ID:1144, to the nucleotide sequence of VGAM209 RNA, herein designated VGAM RNA, also designated SEQ ID:544.

Another function of VGAM209 is therefore inhibition of Solute Carrier Family 16 (monocarboxylic acid transporters), Member 4 (SLC16A4, Accession NM_{—}004696). Accordingly, utilities of VGAM209 include diagnosis, prevention and treatment of diseases and clinical conditions associated with SLC16A4. LOC147180 (Accession XM_{—}097207) is another VGAM209 host target gene. LOC147180 BINDING SITE is HOST TARGET binding site found in the 5′ untranslated region of mRNA encoded by LOC147180, corresponding to a HOST TARGET binding site such as BINDING SITE I, BINDING SITE II or BINDING SITE III. Table 2 illustrates the complementarity of the nucleotide sequences of LOC147180 BINDING SITE, designated SEQ ID:3292, to the nucleotide sequence of VGAM209 RNA, herein designated VGAM RNA, also designated SEQ ID:544.

Another function of VGAM209 is therefore inhibition of LOC147180 (Accession XM_{—}097207). Accordingly, utilities of VGAM209 include diagnosis, prevention and treatment of diseases and clinical conditions associated with LOC147180. LOC148195 (Accession XM_{—}097419) is another VGAM209 host target gene. LOC148195 BINDING SITE is HOST TARGET binding site found in the 5′ untranslated region of mRNA encoded by LOC148195, corresponding to a HOST TARGET binding site such as BINDING SITE I, BINDING SITE II or BINDING SITE III. Table 2 illustrates the complementarity of the nucleotide sequences of LOC148195 BINDING SITE, designated SEQ ID:3297, to the nucleotide sequence of VGAM209 RNA, herein designated VGAM RNA, also designated SEQ ID:544.

Another function of VGAM209 is therefore inhibition of LOC148195 (Accession XM_{—}097419). Accordingly, utilities of VGAM209 include diagnosis, prevention and treatment of diseases and clinical conditions associated with LOC148195. LOC158572 (Accession XM_{—}088608) is another VGAM209 host target gene. LOC158572 BINDING SITE is HOST TARGET binding site found in the 3′ untranslated region of mRNA encoded by LOC158572, corresponding to a HOST TARGET binding site such as BINDING SITE I, BINDING SITE II or BINDING SITE III. Table 2 illustrates the complementarity of the nucleotide sequences of LOC158572 BINDING SITE, designated SEQ ID:3221, to the nucleotide sequence of VGAM209 RNA, herein designated VGAM RNA, also designated SEQ ID:544.

Another function of VGAM209 is therefore inhibition of LOC158572 (Accession XM_{—}088608). Accordingly, utilities of VGAM209 include diagnosis, prevention and treatment of diseases and clinical conditions associated with LOC158572. LOC201595 (Accession XM_{—}114346) is another VGAM209 host target gene. LOC201595 BINDING SITE is HOST TARGET binding site found in the 3′ untranslated region of mRNA encoded by LOC201595, corresponding to a HOST TARGET binding site such as BINDING SITE I, BINDING SITE II or BINDING SITE III. Table 2 illustrates the complementarity of the nucleotide sequences of LOC201595 BINDING SITE, designated SEQ ID:3453, to the nucleotide sequence of VGAM209 RNA, herein designated VGAM RNA, also designated SEQ ID:544.

Another function of VGAM209 is therefore inhibition of LOC201595 (Accession XM_{—}114346). Accordingly, utilities of VGAM209 include diagnosis, prevention and treatment of diseases and clinical conditions associated with LOC201595. LOC203427 (Accession XM_{—}114699) is another VGAM209 host target gene. LOC203427 BINDING SITE is HOST TARGET binding site found in the 5′ untranslated region of mRNA encoded by LOC203427, corresponding to a HOST TARGET binding site such as BINDING SITE I, BINDING SITE II or BINDING SITE III. Table 2 illustrates the complementarity of the nucleotide sequences of LOC203427 BINDING SITE, designated SEQ ID:3464, to the nucleotide sequence of VGAM209 RNA, herein designated VGAM RNA, also designated SEQ ID:544.

Another function of VGAM209 is therefore inhibition of LOC203427 (Accession XM_{—}114699). Accordingly, utilities of VGAM209 include diagnosis, prevention and treatment of diseases and clinical conditions associated with LOC203427. FIG. 1 further provides a conceptual description of a novel bioinformatically detected viral gene of the present invention, referred to here as Viral Genomic Address Messenger 210 (VGAM210) viral gene, which modulates expression of respective host target genes thereof, the function and utility of which host target genes is known in the art.

VGAM210 is a novel bioinformatically detected regulatory, non protein coding, viral micro RNA (miRNA) gene. The method by which VGAM210 was detected is described hereinabove with reference to FIGS. 1-8.

VGAM210 gene, herein designated VGAM GENE, is a viral gene contained in the genome of Vaccinia Virus. VGAM210 host target gene, herein designated VGAM HOST TARGET GENE, is a human gene contained in the human genome.

VGAM210 gene encodes a VGAM210 precursor RNA, herein designated VGAM PRECURSOR RNA. Similar to other miRNA genes, and unlike most ordinary genes, VGAM210 precursor RNA does not encode a protein. A nucleotide sequence identical or highly similar to the nucleotide sequence of VGAM210 precursor RNA is designated SEQ ID:196, and is provided hereinbelow with reference to the sequence listing part. Nucleotide sequence SEQ ID:196 is located at position 110952 relative to the genome of Vaccinia Virus.

VGAM210 precursor RNA folds onto itself, forming VGAM210 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM210 folded precursor RNA into VGAM210 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM210 RNA is designated SEQ ID:545, and is provided hereinbelow with reference to the sequence listing part.

VGAM210 host target gene, herein designated VGAM HOST TARGET GENE, encodes a corresponding messenger RNA, VGAM210 host target RNA, herein designated VGAM HOST TARGET RNA. VGAM210 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM210 RNA, herein designated VGAM RNA, binds complementarily to one or more host target binding sites located in untranslated regions of VGAM210 host target RNA, herein designated VGAM HOST TARGET RNA. This complementary binding is due to the fact that the nucleotide sequence of VGAM210 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM210 RNA, herein designated VGAM RNA, may have a different number of host target binding sites in untranslated regions of a VGAM210 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM210 RNA, herein designated VGAM RNA, to host target binding sites on VGAM210 host target RNA, herein designated VGAM HOST TARGET RNA, such as BINDING SITE I, BINDING SITE II and BINDING SITE III, inhibits translation of VGAM210 host target RNA into VGAM210 host target protein, herein designated VGAM HOST TARGET PROTEIN. VGAM host target protein is therefore outlined by a broken line.

It is appreciated that VGAM210 host target gene, herein designated VGAM HOST TARGET GENE, in fact represents a plurality of VGAM210 host target genes. The mRNA of each one of this plurality of VGAM210 host target genes comprises one or more host target binding sites, each having a nucleotide sequence which is at least partly complementary to VGAM210 RNA, herein designated VGAM RNA, and which when bound by VGAM210 RNA causes inhibition of translation of respective one or more VGAM210 host target proteins.

It is further appreciated by one skilled in the art that the mode of translational inhibition illustrated by FIG. 1 with specific reference to translational inhibition exerted by VGAM210 gene, herein designated VGAM GENE, on one or more VGAM210 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM210 is inhibition of expression of host target genes, as part of a novel viral mechanism of attacking a host. Accordingly, utilities of VGAM210 include diagnosis, prevention and treatment of viral infection by Vaccinia Virus. Specific functions, and accordingly utilities, of VGAM210 correlate with, and may be deduced from, the identity of the host target genes which VGAM210 binds and inhibits, and the function of these host target genes, as elaborated hereinbelow.

Nucleotide sequences of the VGAM210 precursor RNA, herein designated VGAM PRECURSOR RNA, and of the ‘diced’ VGAM210 RNA, herein designated VGAM RNA, and a schematic representation of the secondary folding of VGAM210 folded precursor RNA, herein designated VGAM FOLDED PRECURSOR RNA, of VGAM210 are further described hereinbelow with reference to Table 1.

Nucleotide sequences of host target binding sites, such as BINDING SITE-I, BINDING SITE-II and BINDING SITE-III of FIG. 1, found on VGAM210 host target RNA, and schematic representation of the complementarity of each of these host target binding sites to VGAM210 RNA, herein designated VGAM RNA, are described hereinbelow with reference to Table 2.

As mentioned hereinabove with reference to FIG. 1, a function of VGAM210 gene, herein designated VGAM is inhibition of expression of VGAM210 target genes. It is appreciated that specific functions, and accordingly utilities, of VGAM210 correlate with, and may be deduced from, the identity of the target genes which VGAM210 binds and inhibits, and the function of these target genes, as elaborated hereinbelow.

Phosphodiesterase 1A, Calmodulin-dependent (PDE1A, Accession NM_{—}005019) is a VGAM210 host target gene. PDE1A BINDING SITE is HOST TARGET binding site found in the 3′ untranslated region of mRNA encoded by PDE1A, corresponding to a HOST TARGET binding site such as BINDING SITE I, BINDING SITE II or BINDING SITE III. Table 2 illustrates the complementarity of the nucleotide sequences of PDE1A BINDING SITE, designated SEQ ID:1171, to the nucleotide sequence of VGAM210 RNA, herein designated VGAM RNA, also designated SEQ ID:545.

A function of VGAM210 is therefore inhibition of Phosphodiesterase 1A, Calmodulin-dependent (PDE1A, Accession NM_{—}005019), a gene which is a Ca2+-calmodulin dependent cyclic nucleotide phosphodiesterase and has a higher affinity for cgmp than for camp. Accordingly, utilities of VGAM210 include diagnosis, prevention and treatment of diseases and clinical conditions associated with PDE1A. The function of PDE1A has been established by previous studies. Phosphodiesterase 1 is a membrane-bound exonuclease that hydrolyzes phosphodiester bonds. See 171885. Wilson and McKenna (1988) examined the segregation of the gene for human phosphodiesterase 1A in human-rodent somatic cell hybrids. Electrophoretic analysis of phosphodiesterase 1A in hybrids suggested that the enzyme is a monomer. The PDE1A gene segregated concordantly with human chromosome 4 in all but 1 of 26 hybrids examined and showed 4 or more instances of discordance with all other chromosomes. By screening a hippocampus library with a bovine 61-kD CaM PDE cDNA, Loughney et al. (1996) isolated cDNAs encoding PDE1A (HCAM1) and PDE1C (HCAM3; 602987). The sequence of the predicted 535-amino acid protein is 94% identical to that of the bovine 61-kD CaM PDE when 2 short regions unique to PDE1A are excluded from comparison. Northern blot analysis revealed tissue-specific expression of 4.8-, 2.4-, and 2.6-kb PDE1A mRNAs, with transcripts most abundant in brain, heart, kidney, and skeletal muscle. Although expression of full-length PDE1A in S. cerevisiae did not result in PDE activity, an amino-truncated protein gave measurable PDE activity with higher affinity for cGMP than for cAMP.

Full details of the abovementioned studies are described in the following publications, the disclosure of which are hereby incorporated by reference:
Loughney, K.; Martins, T. J.; Harris, E. A. S.; Sadhu, K.; Hicks, J. B.; Sonnenburg, W. K.; Beavo, J. A.; Ferguson, K.: Isolation and characterization of cDNAs corresponding to two human calcium, calmodulin-regulated, 3-prime, 5-prime-cyclic nucleotide phosphodiesterases. J. Biol. Chem. 271: 796-806, 1996.; and
Wilson, D. E.; McKenna, L.: Assignment of the human gene for phosphodiesterase 1A to chromosome 4. (Abstract) Am. J. Hum. Genet. 43: A162 only, 1988.

Further studies establishing the function and utilities of PDE1A are found in John Hopkins OMIM database record ID 171890, and in sited publications numbered 2987-2988 listed in the bibliography section hereinbelow, which are also hereby incorporated by reference. ElaC Homolog 1 (E. coli) (ELAC1, Accession XM_{—}165659) is another VGAM210 host target gene. ELAC1 BINDING SITE is HOST TARGET binding site found in the 3′ untranslated region of mRNA encoded by ELAC1, corresponding to a HOST TARGET binding site such as BINDING SITE I, BINDING SITE II or BINDING SITE III. Table 2 illustrates the complementarity of the nucleotide sequences of ELAC1 BINDING SITE, designated SEQ ID:3506, to the nucleotide sequence of VGAM210 RNA, herein designated VGAM RNA, also designated SEQ ID:545.

Another function of VGAM210 is therefore inhibition of ElaC Homolog 1 (E. coli) (ELAC1, Accession XM_{—}165659). Accordingly, utilities of VGAM210 include diagnosis, prevention and treatment of diseases and clinical conditions associated with ELAC1. LOC154790 (Accession XM_{—}088044) is another VGAM210 host target gene. LOC154790 BINDING SITE is HOST TARGET binding site found in the 5′ untranslated region of mRNA encoded by LOC154790, corresponding to a HOST TARGET binding site such as BINDING SITE I, BINDING SITE II or BINDING SITE III. Table 2 illustrates the complementarity of the nucleotide sequences of LOC154790 BINDING SITE, designated SEQ ID:3191, to the nucleotide sequence of VGAM210 RNA, herein designated VGAM RNA, also designated SEQ ID:545.

Another function of VGAM210 is therefore inhibition of LOC154790 (Accession XM_{—}088044). Accordingly, utilities of VGAM210 include diagnosis, prevention and treatment of diseases and clinical conditions associated with LOC154790. LOC158434 (Accession XM_{—}098939) is another VGAM210 host target gene. LOC158434 BINDING SITE is HOST TARGET binding site found in the 3′ untranslated region of mRNA encoded by LOC158434, corresponding to a HOST TARGET binding site such as BINDING SITE I, BINDING SITE II or BINDING SITE III. Table 2 illustrates the complementarity of the nucleotide sequences of LOC158434 BINDING SITE, designated SEQ ID:3389, to the nucleotide sequence of VGAM210 RNA, herein designated VGAM RNA, also designated SEQ ID:545.

Another function of VGAM210 is therefore inhibition of LOC158434 (Accession XM_{—}098939). Accordingly, utilities of VGAM210 include diagnosis, prevention and treatment of diseases and clinical conditions associated with LOC158434. FIG. 1 further provides a conceptual description of a novel bioinformatically detected viral gene of the present invention, referred to here as Viral Genomic Address Messenger 211 (VGAM211) viral gene, which modulates expression of respective host target genes thereof, the function and utility of which host target genes is known in the art.

VGAM211 is a novel bioinformatically detected regulatory, non protein coding, viral micro RNA (miRNA) gene. The method by which VGAM211 was detected is described hereinabove with reference to FIGS. 1-8.

VGAM211 gene, herein designated VGAM GENE, is a viral gene contained in the genome of Vaccinia Virus. VGAM211 host target gene, herein designated VGAM HOST TARGET GENE, is a human gene contained in the human genome.

VGAM211 gene encodes a VGAM211 precursor RNA, herein designated VGAM PRECURSOR RNA. Similar to other miRNA genes, and unlike most ordinary genes, VGAM211 precursor RNA does not encode a protein. A nucleotide sequence identical or highly similar to the nucleotide sequence of VGAM211 precursor RNA is designated SEQ ID:197, and is provided hereinbelow with reference to the sequence listing part. Nucleotide sequence SEQ ID:197 is located at position 110025 relative to the genome of Vaccinia Virus.

VGAM211 precursor RNA folds onto itself, forming VGAM211 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM211 folded precursor RNA into VGAM211 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM211 RNA is designated SEQ ID:546, and is provided hereinbelow with reference to the sequence listing part.

VGAM211 host target gene, herein designated VGAM HOST TARGET GENE, encodes a corresponding messenger RNA, VGAM211 host target RNA, herein designated VGAM HOST TARGET RNA. VGAM211 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM211 RNA, herein designated VGAM RNA, binds complementarily to one or more host target binding sites located in untranslated regions of VGAM211 host target RNA, herein designated VGAM HOST TARGET RNA. This complementary binding is due to the fact that the nucleotide sequence of VGAM211 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM211 RNA, herein designated VGAM RNA, may have a different number of host target binding sites in untranslated regions of a VGAM211 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM211 RNA, herein designated VGAM RNA, to host target binding sites on VGAM211 host target RNA, herein designated VGAM HOST TARGET RNA, such as BINDING SITE I, BINDING SITE II and BINDING SITE III, inhibits translation of VGAM211 host target RNA into VGAM211 host target protein, herein designated VGAM HOST TARGET PROTEIN. VGAM host target protein is therefore outlined by a broken line.

It is appreciated that VGAM211 host target gene, herein designated VGAM HOST TARGET GENE, in fact represents a plurality of VGAM211 host target genes. The mRNA of each one of this plurality of VGAM211 host target genes comprises one or more host target binding sites, each having a nucleotide sequence which is at least partly complementary to VGAM211 RNA, herein designated VGAM RNA, and which when bound by VGAM211 RNA causes inhibition of translation of respective one or more VGAM211 host target proteins.

It is further appreciated by one skilled in the art that the mode of translational inhibition illustrated by FIG. 1 with specific reference to translational inhibition exerted by VGAM211 gene, herein designated VGAM GENE, on one or more VGAM211 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM211 is inhibition of expression of host target genes, as part of a novel viral mechanism of attacking a host. Accordingly, utilities of VGAM211 include diagnosis, prevention and treatment of viral infection by Vaccinia Virus. Specific functions, and accordingly utilities, of VGAM211 correlate with, and may be deduced from, the identity of the host target genes which VGAM211 binds and inhibits, and the function of these host target genes, as elaborated hereinbelow.

Nucleotide sequences of the VGAM211 precursor RNA, herein designated VGAM PRECURSOR RNA, and of the ‘diced’ VGAM211 RNA, herein designated VGAM RNA, and a schematic representation of the secondary folding of VGAM211 folded precursor RNA, herein designated VGAM FOLDED PRECURSOR RNA, of VGAM211 are further described hereinbelow with reference to Table 1.

Nucleotide sequences of host target binding sites, such as BINDING SITE-I, BINDING SITE-II and BINDING SITE-III of FIG. 1, found on VGAM211 host target RNA, and schematic representation of the complementarity of each of these host target binding sites to VGAM211 RNA, herein designated VGAM RNA, are described hereinbelow with reference to Table 2.

As mentioned hereinabove with reference to FIG. 1, a function of VGAM211 gene, herein designated VGAM is inhibition of expression of VGAM211 target genes. It is appreciated that specific functions, and accordingly utilities, of VGAM211 correlate with, and may be deduced from, the identity of the target genes which VGAM211 binds and inhibits, and the function of these target genes, as elaborated hereinbelow.

Mitogen-activated Protein Kinase Kinase Kinase 5 (MAP3K5, Accession NM_{—}005923) is a VGAM211 host target gene. MAP3K5 BINDING SITE is HOST TARGET binding site found in the 3′ untranslated region of mRNA encoded by MAP3K5, corresponding to a HOST TARGET binding site such as BINDING SITE I, BINDING SITE II or BINDING SITE III. Table 2 illustrates the complementarity of the nucleotide sequences of MAP3K5 BINDING SITE, designated SEQ ID: 1261, to the nucleotide sequence of VGAM211 RNA, herein designated VGAM RNA, also designated SEQ ID:546.

A function of VGAM211 is therefore inhibition of Mitogen-activated Protein Kinase Kinase Kinase 5 (MAP3K5, Accession NM_{—}005923), a gene which phosphorylates and activates two different subgroups of map kinase kinases, mkk4/sek1 and mkk3/mapkk6 (or mkk6). overexpression induces apoptotic cell death. Accordingly, utilities of VGAM211 include diagnosis, prevention and treatment of diseases and clinical conditions associated with MAP3K5. The function of MAP3K5 has been established by previous studies. Mitogen-activated protein kinase (MAPK) signaling cascades include MAPK or extracellular signal-regulated kinase (ERK), MAPK kinase (MAP2K, also called MKK or MEK), and MAPK kinase kinase (MAP3K, also called MAPKKK or MEKK). MAPKK kinase/MEKK phosphorylates and activates its downstream protein kinase, MAPK kinase/MEK, which in turn activates MAPK. The kinases of these signaling cascades are highly conserved, and homologs exist in yeast, Drosophila, and mammalian cells Ichijo et al. (1997) used a similar cloning strategy to identify a nearly identical MAPKKK cDNA, termed ASK1 for apoptosis signal-regulating kinase. The deduced protein contains 1,375 amino acids, and is most closely related to yeast SSK2 and SSK22, which are upstream regulators of yeast HOG1 MAPK. ASK1 expression complements a yeast mutant lacking functional SSK2 and SSK22. ASK1 also activates MKK3 (OMIM Ref. No. 602315), MKK4 (SEK1), and MKK6 (OMIM Ref. No. 601254). Overexpression of ASK1 induces apoptotic cell death, and ASK1 is activated in cells treated with tumor necrosis factor-alpha (TNFA; 191160). Nishitoh et al. (1998) showed that ASK1 interacts with members of the TRAF family and is activated by TRAF2 (OMIM Ref. No. 601895) in the TNF-signaling pathway. After activation by TRAF2, ASK1 activates MKK4, which in turn activates JNK. Thus, ASK1 is a mediator of TRAF2-induced JNK activation. Animal model experiments lend further support to the function of MAP3K5. Using a forward genetic screen of C. elegans mutants, Kim et al. (2002) showed that viable worms lacking esp2 and esp8, homologs of the mammalian MAP kinases SEK1 and ASK1, were highly susceptible to and died more rapidly from both a gram-negative bacterium, P. aeruginosa, and a gram-positive organism, E. faecalis, than wildtype worms. RNA-interference and biochemical analyses likewise implicated the p38 MAP kinase homolog, pmk1, in susceptibility to these pathogens. Kim et al. (2002) concluded that MAP kinase signaling, which is also involved in plant pathogen resistance, is a conserved element in innate metazoan immunity to diverse pathogens.

It is appreciated that the abovementioned animal model for MAP3K5 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Kim, D. H.; Feinbaum, R.; Alloing, G.; Emerson, F. E.; Garsin, D. A.; Inoue, H.; Tanaka-Hino, M.; Hisamoto, N.; Matsumoto, K.; Tan, M.-W.; Ausubel, F. M.: A conserved p38 MAP kinase pathway in Caenorhabditis elegans innate immunity. Science 297: 623-626, 2002.; and
Nishitoh, H.; Saitoh, M.; Mochida, Y.; Takeda, K.; Nakano, H.; Rothe, M.; Miyazono, K.; Ichijo, H.: ASK1 is essential for JNK/SAPK activation by TRAF2. Molec. Cell 2: 389-395, 1998.

Further studies establishing the function and utilities of MAP3K5 are found in John Hopkins OMIM database record ID 602448, and in sited publications numbered 1352-1354, 2295, 3027, 2366-2367, 146 and 1472 listed in the bibliography section hereinbelow, which are also hereby incorporated by reference. DKFZP564F0522 (Accession XM_{—}043885) is another VGAM211 host target gene. DKFZP564F0522 BINDING SITE is HOST TARGET binding site found in the 3′ untranslated region of mRNA encoded by DKFZP564F0522, corresponding to a HOST TARGET binding site such as BINDING SITE I, BINDING SITE II or BINDING SITE III. Table 2 illustrates the complementarity of the nucleotide sequences of DKFZP564F0522 BINDING SITE, designated SEQ ID:2825, to the nucleotide sequence of VGAM211 RNA, herein designated VGAM RNA, also designated SEQ ID:546.

Another function of VGAM211 is therefore inhibition of DKFZP564F0522 (Accession XM_{—}043885). Accordingly, utilities of VGAM211 include diagnosis, prevention and treatment of diseases and clinical conditions associated with DKFZP564F0522. GPCR150 (Accession NM_{—}014373) is another VGAM211 host target gene. GPCR150 BINDING SITE is HOST TARGET binding site found in the 3′ untranslated region of mRNA encoded by GPCR150, corresponding to a HOST TARGET binding site such as BINDING SITE I, BINDING SITE II or BINDING SITE III. Table 2 illustrates the complementarity of the nucleotide sequences of GPCR150 BINDING SITE, designated SEQ ID: 1496, to the nucleotide sequence of VGAM211 RNA, herein designated VGAM RNA, also designated SEQ ID:546.

Another function of VGAM211 is therefore inhibition of GPCR150 (Accession NM_{—}014373). Accordingly, utilities of VGAM211 include diagnosis, prevention and treatment of diseases and clinical conditions associated with GPCR150. Solute Carrier Family 26, Member 7 (SLC26A7, Accession NM_{—}052832) is another VGAM211 host target gene. SLC26A7 BINDING SITE is HOST TARGET binding site found in the 3′ untranslated region of mRNA encoded by SLC26A7, corresponding to a HOST TARGET binding site such as BINDING SITE I, BINDING SITE II or BINDING SITE III. Table 2 illustrates the complementarity of the nucleotide sequences of SLC26A7 BINDING SITE, designated SEQ ID:2340, to the nucleotide sequence of VGAM211 RNA, herein designated VGAM RNA, also designated SEQ ID:546.

Another function of VGAM211 is therefore inhibition of Solute Carrier Family 26, Member 7 (SLC26A7, Accession NM_{—}052832). Accordingly, utilities of VGAM211 include diagnosis, prevention and treatment of diseases and clinical conditions associated with SLC26A7. Solute Carrier Family 6 (neurotransmitter transporter), Member 14 (SLC6A14, Accession NM_{—}007231) is another VGAM211 host target gene. SLC6A14 BINDING SITE is HOST TARGET binding site found in the 3′ untranslated region of mRNA encoded by SLC6A14, corresponding to a HOST TARGET binding site such as BINDING SITE I, BINDING SITE II or BINDING SITE III. Table 2 illustrates the complementarity of the nucleotide sequences of SLC6A14 BINDING SITE, designated SEQ ID:1369, to the nucleotide sequence of VGAM211 RNA, herein designated VGAM RNA, also designated SEQ ID:546.

Another function of VGAM211 is therefore inhibition of Solute Carrier Family 6 (neurotransmitter transporter), Member 14 (SLC6A14, Accession NM_{—}007231). Accordingly, utilities of VGAM211 include diagnosis, prevention and treatment of diseases and clinical conditions associated with SLC6A14. LOC116228 (Accession XM_{—}057659) is another VGAM211 host target gene. LOC116228 BINDING SITE is HOST TARGET binding site found in the 3′ untranslated region of mRNA encoded by LOC116228, corresponding to a HOST TARGET binding site such as BINDING SITE I, BINDING SITE II or BINDING SITE III. Table 2 illustrates the complementarity of the nucleotide sequences of LOC116228 BINDING SITE, designated SEQ ID:2980, to the nucleotide sequence of VGAM211 RNA, herein designated VGAM RNA, also designated SEQ ID:546.

Another function of VGAM211 is therefore inhibition of LOC116228 (Accession XM_{—}057659). Accordingly, utilities of VGAM211 include diagnosis, prevention and treatment of diseases and clinical conditions associated with LOC116228. FIG. 1 further provides a conceptual description of a novel bioinformatically detected viral gene of the present invention, referred to here as Viral Genomic Address Messenger 212 (VGAM212) viral gene, which modulates expression of respective host target genes thereof, the function and utility of which host target genes is known in the art.

VGAM212 is a novel bioinformatically detected regulatory, non protein coding, viral micro RNA (miRNA) gene. The method by which VGAM212 was detected is described hereinabove with reference to FIGS. 1-8.

VGAM212 gene, herein designated VGAM GENE, is a viral gene contained in the genome of Vaccinia Virus. VGAM212 host target gene, herein designated VGAM HOST TARGET GENE, is a human gene contained in the human genome.

VGAM212 gene encodes a VGAM212 precursor RNA, herein designated VGAM PRECURSOR RNA. Similar to other miRNA genes, and unlike most ordinary genes, VGAM212 precursor RNA does not encode a protein. A nucleotide sequence identical or highly similar to the nucleotide sequence of VGAM212 precursor RNA is designated SEQ ID:198, and is provided hereinbelow with reference to the sequence listing part. Nucleotide sequence SEQ ID:198 is located at position 111922 relative to the genome of Vaccinia Virus.

VGAM212 precursor RNA folds onto itself, forming VGAM212 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM212 folded precursor RNA into VGAM212 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM212 RNA is designated SEQ ID:547, and is provided hereinbelow with reference to the sequence listing part.

VGAM212 host target gene, herein designated VGAM HOST TARGET GENE, encodes a corresponding messenger RNA, VGAM212 host target RNA, herein designated VGAM HOST TARGET RNA. VGAM212 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM212 RNA, herein designated VGAM RNA, binds complementarily to one or more host target binding sites located in untranslated regions of VGAM212 host target RNA, herein designated VGAM HOST TARGET RNA. This complementary binding is due to the fact that the nucleotide sequence of VGAM212 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM212 RNA, herein designated VGAM RNA, may have a different number of host target binding sites in untranslated regions of a VGAM212 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM212 RNA, herein designated VGAM RNA, to host target binding sites on VGAM212 host target RNA, herein designated VGAM HOST TARGET RNA, such as BINDING SITE I, BINDING SITE II and BINDING SITE III, inhibits translation of VGAM212 host target RNA into VGAM212 host target protein, herein designated VGAM HOST TARGET PROTEIN. VGAM host target protein is therefore outlined by a broken line.

It is appreciated that VGAM212 host target gene, herein designated VGAM HOST TARGET GENE, in fact represents a plurality of VGAM212 host target genes. The mRNA of each one of this plurality of VGAM212 host target genes comprises one or more host target binding sites, each having a nucleotide sequence which is at least partly complementary to VGAM212 RNA, herein designated VGAM RNA, and which when bound by VGAM212 RNA causes inhibition of translation of respective one or more VGAM212 host target proteins.

It is further appreciated by one skilled in the art that the mode of translational inhibition illustrated by FIG. 1 with specific reference to translational inhibition exerted by VGAM212 gene, herein designated VGAM GENE, on one or more VGAM212 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM212 is inhibition of expression of host target genes, as part of a novel viral mechanism of attacking a host. Accordingly, utilities of VGAM212 include diagnosis, prevention and treatment of viral infection by Vaccinia Virus. Specific functions, and accordingly utilities, of VGAM212 correlate with, and may be deduced from, the identity of the host target genes which VGAM212 binds and inhibits, and the function of these host target genes, as elaborated hereinbelow.

Nucleotide sequences of the VGAM212 precursor RNA, herein designated VGAM PRECURSOR RNA, and of the ‘diced’ VGAM212 RNA, herein designated VGAM RNA, and a schematic representation of the secondary folding of VGAM212 folded precursor RNA, herein designated VGAM FOLDED PRECURSOR RNA, of VGAM212 are further described hereinbelow with reference to Table 1.

Nucleotide sequences of host target binding sites, such as BINDING SITE-I, BINDING SITE-II and BINDING SITE-III of FIG. 1, found on VGAM212 host target RNA, and schematic representation of the complementarity of each of these host target binding sites to VGAM212 RNA, herein designated VGAM RNA, are described hereinbelow with reference to Table 2.

As mentioned hereinabove with reference to FIG. 1, a function of VGAM212 gene, herein designated VGAM is inhibition of expression of VGAM212 target genes. It is appreciated that specific functions, and accordingly utilities, of VGAM212 correlate with, and may be deduced from, the identity of the target genes which VGAM212 binds and inhibits, and the function of these target genes, as elaborated hereinbelow.

Cyclin C (CCNC, Accession NM_{—}005190) is a VGAM212 host target gene. CCNC BINDING SITE is HOST TARGET binding site found in the 3′ untranslated region of mRNA encoded by CCNC, corresponding to a HOST TARGET binding site such as BINDING SITE I, BINDING SITE II or BINDING SITE III. Table 2 illustrates the complementarity of the nucleotide sequences of CCNC BINDING SITE, designated SEQ ID:1192, to the nucleotide sequence of VGAM212 RNA, herein designated VGAM RNA, also designated SEQ ID:547.

A function of VGAM212 is therefore inhibition of Cyclin C (CCNC, Accession NM_{—}005190), a gene which is involved in the control of cell cycle and can regulate transcription by targeting the CDK7/cyclin H subunits of the general transcription initiation factor IIH. Accordingly, utilities of VGAM212 include diagnosis, prevention and treatment of diseases and clinical conditions associated with CCNC. The function of CCNC has been established by previous studies. See 123837. Lew et al. (1991) isolated a number of cDNAs derived from human mRNAs that were able to substitute for G1 cyclin genes in S. cerevisiae. Several of these encoded human cyclins A, B1, and B2. Three novel genes were identified, which they called cyclins C, D, and E. The 3 novel proteins were sufficiently distantly related to the other members of the cyclin family and to each other as to constitute 3 new classes of cyclins. Cyclin C and E mRNAs accumulated periodically during the cell cycle, peaking at different times in G1. Mammalian CDK8 (OMIM Ref. No. 603184) and cyclin C are components of the RNA polymerase II holoenzyme complex, where they function as a protein kinase that phosphorylates the C-terminal domain of the largest subunit of RNA polymerase II. The CDK8/cyclin C protein complex is also found in a number of mammalian ‘Mediator’-like protein complexes, which repress activated transcription independently of the C-terminal domain in vitro. Akoulitchev et al. (2000) demonstrated that CDK8/cyclin C can regulate transcription by targeting the CDK7 (OMIM Ref. No. 601955)/cyclin H (OMIM Ref. No. 601953) subunits of the general transcription initiation factor IIH (OMIM Ref. No. 189972). CDK8 phosphorylates mammalian cyclin H at serine 5 and serine 304 both in vitro and in vivo, in the vicinity of its functionally unique N- and C-terminal alpha-helical domains. This phosphorylation represses both the ability of TFIIH to activate transcription and its C-terminal kinase activity. In addition, mimicking CDK8 phosphorylation of cyclin H in vivo has a dominant-negative effect on cell growth. Akoulitchev et al. (2000) concluded that their results linked the Mediator complex and the basal transcription machinery by a regulatory pathway involving 2 cyclin-dependent kinases. This pathway appears to be unique to higher organisms.

Full details of the abovementioned studies are described in the following publications, the disclosure of which are hereby incorporated by reference:
Lew, D. J.; Dulic, V.; Reed, S. I.: Isolation of three novel human cyclins by rescue of G1 cyclin (Cln) function in yeast. Cell 66: 1197-1206, 1991.; and
Akoulitchev, S.; Chuikov, S.; Reinberg, D.: TFIIH is negatively regulated by cdk8-containing mediator complexes. Nature 407: 102-106, 2000.

Further studies establishing the function and utilities of CCNC are found in John Hopkins OMIM database record ID 123838, and in sited publications numbered 105 and 1054-1055 listed in the bibliography section hereinbelow, which are also hereby incorporated by reference. Male-specific Lethal 3-like 1 (Drosophila) (MSL3Li, Accession NM_{—}006800) is another VGAM212 host target gene. MSL3L1 BINDING SITE is HOST TARGET binding site found in the 5′ untranslated region of mRNA encoded by MSL3Li, corresponding to a HOST TARGET binding site such as BINDING SITE I, BINDING SITE II or BINDING SITE III. Table 2 illustrates the complementarity of the nucleotide sequences of MSL3L1 BINDING SITE, designated SEQ ID:1331, to the nucleotide sequence of VGAM212 RNA, herein designated VGAM RNA, also designated SEQ ID: 547.

Another function of VGAM212 is therefore inhibition of Male-specific Lethal 3-like 1 (Drosophila) (MSL3Li, Accession NM_{—}006800). Accordingly, utilities of VGAM212 include diagnosis, prevention and treatment of diseases and clinical conditions associated with MSL3L1. Thrombospondin 1 (THBS1, Accession NM_{—}003246) is another VGAM212 host target gene. THBS1 BINDING SITE is HOST TARGET binding site found in the 3′ untranslated region of mRNA encoded by THBS1, corresponding to a HOST TARGET binding site such as BINDING SITE I, BINDING SITE II or BINDING SITE III. Table 2 illustrates the complementarity of the nucleotide sequences of THBS1 BINDING SITE, designated SEQ ID: 1004, to the nucleotide sequence of VGAM212 RNA, herein designated VGAM RNA, also designated SEQ ID:547.

Another function of VGAM212 is therefore inhibition of Thrombospondin 1 (THBS1, Accession NM_{—}003246), a gene which is a member of a family of adhesive molecules, involves in blood clotting and in angiogenesis. Accordingly, utilities of VGAM212 include diagnosis, prevention and treatment of diseases and clinical conditions associated with THBS1. The function of THBS1 has been established by previous studies. Natural inhibitors of angiogenesis are able to block pathologic neovascularization without harming the preexisting vasculature. Volpert et al. (2002) demonstrated that 2 such inhibitors, thrombospondin I and pigment epithelium-derived factor (OMIM Ref. No. 172860), derive specificity for remodeling vessels from their dependence on Fas/Fas ligand (134637; 134638)-mediated apoptosis to block angiogenesis. Both inhibitors up-regulated FasL on endothelial cells. Expression of the essential partner of FasL, Fas receptor, was low on quiescent endothelial cells and vessels but greatly enhanced by inducers of angiogenesis, thereby specifically sensitizing the stimulated cells to apoptosis by inhibitor-generated FasL. The antiangiogenic activity of thrombospondin I and pigment epithelium-derived factor both in vitro and in vivo was dependent on this dual induction of Fas and FasL and the resulting apoptosis. Volpert et al. (2002) concluded that this example of cooperation between pro- and antiangiogenic factors in the inhibition of angiogenesis provides one explanation for the ability of inhibitors to select remodeling capillaries for destruction. Animal model experiments lend further support to the function of THBS1. To explore the function of thrombospondin I in vivo, Lawler et al. (1998) disrupted the Thbs1 gene by homologous recombination in the mouse genome. Platelets from these mice were completely deficient in Thbs1 protein; however, thrombin-induced platelet aggregation was not diminished. The deficient mice displayed a mild and variable lordotic curvature of the spine that was apparent from birth. They also displayed an increase in the number of circulating white blood cells, with monocytes and eosinophils having the largest percent increases. Although other major organs showed no abnormalities consistent with high levels of expression of Thbs1 in lung, Lawler et al. (1998) observed abnormalities in the lungs of the mice lacking Thbs1. Extensive acute and organizing pneumonia with neutrophils and macrophages developed by 4 weeks of age. The macrophages stained for hemosiderin, indicating that diffuse alveolar hemorrhage was occurring. Later, the number of neutrophils decreased and a striking increase in the number of hemosiderin-containing macrophages was observed associated with multiple-lineage epithelial hyperplasia and the deposition of collagen and elastin. The results indicated that THBS1 is involved in normal lung homeostasis.

It is appreciated that the abovementioned animal model for THBS1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Lawler, J.; Sunday, M.; Thibert, V.; Duquette, M.; George, E. L.; Rayburn, H.; Hynes, R. O.: Thrombospondin-1 is required for normal murine pulmonary homeostasis and its absence causes pneumonia. J. Clin. Invest. 101: 982-992, 1998.; and
Volpert, O. V.; Zaichuk, T.; Zhou, W.; Reiher, F.; Ferguson, T. A.; Stuart, P. M.; Amin, M.; Bouck, N. P.: Inducer-stimulated Fas targets activated endothelium for destruction by anti-a.

Further studies establishing the function and utilities of THBS1 are found in John Hopkins OMIM database record ID 188060, and in sited publications numbered 2392-2398, 7 and 2399 listed in the bibliography section hereinbelow, which are also hereby incorporated by reference. Cysteine and Glycine-rich Protein 3 (cardiac LIM protein) (CSRP3, Accession NM_{—}003476) is another VGAM212 host target gene. CSRP3 BINDING SITE is HOST TARGET binding site found in the 3′ untranslated region of mRNA encoded by CSRP3, corresponding to a HOST TARGET binding site such as BINDING SITE I, BINDING SITE II or BINDING SITE III. Table 2 illustrates the complementarity of the nucleotide sequences of CSRP3 BINDING SITE, designated SEQ ID:1031, to the nucleotide sequence of VGAM212 RNA, herein designated VGAM RNA, also designated SEQ ID:547.

Another function of VGAM212 is therefore inhibition of Cysteine and Glycine-rich Protein 3 (cardiac LIM protein) (CSRP3, Accession NM_{—}003476). Accordingly, utilities of VGAM212 include diagnosis, prevention and treatment of diseases and clinical conditions associated with CSRP3. FLJ11181 (Accession NM_{—}018350) is another VGAM212 host target gene. FLJ11181 BINDING SITE is HOST TARGET binding site found in the 5′ untranslated region of mRNA encoded by FLJ11181, corresponding to a HOST TARGET binding site such as BINDING SITE I, BINDING SITE II or BINDING SITE III. Table 2 illustrates the complementarity of the nucleotide sequences of FLJ11181 BINDING SITE, designated SEQ ID:1817, to the nucleotide sequence of VGAM212 RNA, herein designated VGAM RNA, also designated SEQ ID:547.

Another function of VGAM212 is therefore inhibition of FLJ11181 (Accession NM_{—}018350). Accordingly, utilities of VGAM212 include diagnosis, prevention and treatment of diseases and clinical conditions associated with FLJ11181. FLJ23132 (Accession XM_{—}171194) is another VGAM212 host target gene. FLJ23132 BINDING SITE is HOST TARGET binding site found in the 3′ untranslated region of mRNA encoded by FLJ23132, corresponding to a HOST TARGET binding site such as BINDING SITE I, BINDING SITE II or BINDING SITE III. Table 2 illustrates the complementarity of the nucleotide sequences of FLJ23132 BINDING SITE, designated SEQ ID:3692, to the nucleotide sequence of VGAM212 RNA, herein designated VGAM RNA, also designated SEQ ID:547.

Another function of VGAM212 is therefore inhibition of FLJ23132 (Accession XM_{—}171194). Accordingly, utilities of VGAM212 include diagnosis, prevention and treatment of diseases and clinical conditions associated with FLJ23132. GRB2-associated Binding Protein 3 (GAB3, Accession NM_{—}080612) is another VGAM212 host target gene. GAB3 BINDING SITE is HOST TARGET binding site found in the 3′ untranslated region of mRNA encoded by GAB3, corresponding to a HOST TARGET binding site such as BINDING SITE I, BINDING SITE II or BINDING SITE III. Table 2 illustrates the complementarity of the nucleotide sequences of GAB3 BINDING SITE, designated SEQ ID:2378, to the nucleotide sequence of VGAM212 RNA, herein designated VGAM RNA, also designated SEQ ID:547.

Another function of VGAM212 is therefore inhibition of GRB2-associated Binding Protein 3 (GAB3, Accession NM_{—}080612). Accordingly, utilities of VGAM212 include diagnosis, prevention and treatment of diseases and clinical conditions associated with GAB3. MGC13033 (Accession NM_{—}031447) is another VGAM212 host target gene. MGC13033 BINDING SITE is HOST TARGET binding site found in the 3′ untranslated region of mRNA encoded by MGC13033, corresponding to a HOST TARGET binding site such as BINDING SITE I, BINDING SITE II or BINDING SITE III. Table 2 illustrates the complementarity of the nucleotide sequences of MGC13033 BINDING SITE, designated SEQ ID:2198, to the nucleotide sequence of VGAM212 RNA, herein designated VGAM RNA, also designated SEQ ID:547.

Another function of VGAM212 is therefore inhibition of MGC13033 (Accession NM_{—}031447). Accordingly, utilities of VGAM212 include diagnosis, prevention and treatment of diseases and clinical conditions associated with MGC13033. PRO0386 (Accession NM_{—}018562) is another VGAM212 host target gene. PRO0386 BINDING SITE is HOST TARGET binding site found in the 5′ untranslated region of mRNA encoded by PRO0386, corresponding to a HOST TARGET binding site such as BINDING SITE I, BINDING SITE II or BINDING SITE III. Table 2 illustrates the complementarity of the nucleotide sequences of PRO0386 BINDING SITE, designated SEQ ID:1844, to the nucleotide sequence of VGAM212 RNA, herein designated VGAM RNA, also designated SEQ ID:547.

Another function of VGAM212 is therefore inhibition of PRO0386 (Accession NM_{—}018562). Accordingly, utilities of VGAM212 include diagnosis, prevention and treatment of diseases and clinical conditions associated with PRO0386. LOC149351 (Accession XM_{—}086503) is another VGAM212 host target gene. LOC149351 BINDING SITE is HOST TARGET binding site found in the 5′ untranslated region of mRNA encoded by LOC149351, corresponding to a HOST TARGET binding site such as BINDING SITE I, BINDING SITE II or BINDING SITE III. Table 2 illustrates the complementarity of the nucleotide sequences of LOC149351 BINDING SITE, designated SEQ ID:3132, to the nucleotide sequence of VGAM212 RNA, herein designated VGAM RNA, also designated SEQ ID:547.

Another function of VGAM212 is therefore inhibition of LOC149351 (Accession XM_{—}086503). Accordingly, utilities of VGAM212 include diagnosis, prevention and treatment of diseases and clinical conditions associated with LOC149351. LOC163590 (Accession NM_{—}145034) is another VGAM212 host target gene. LOC163590 BINDING SITE is HOST TARGET binding site found in the 3′ untranslated region of mRNA encoded by LOC163590, corresponding to a HOST TARGET binding site such as BINDING SITE I, BINDING SITE II or BINDING SITE III. Table 2 illustrates the complementarity of the nucleotide sequences of LOC163590 BINDING SITE, designated SEQ ID:2512, to the nucleotide sequence of VGAM212 RNA, herein designated VGAM RNA, also designated SEQ ID:547.

Another function of VGAM212 is therefore inhibition of LOC163590 (Accession NM_{—}145034). Accordingly, utilities of VGAM212 include diagnosis, prevention and treatment of diseases and clinical conditions associated with LOC163590. LOC256307 (Accession XM_{—}173118) is another VGAM212 host target gene. LOC256307 BINDING SITE is HOST TARGET binding site found in the 3′ untranslated region of mRNA encoded by LOC256307, corresponding to a HOST TARGET binding site such as BINDING SITE I, BINDING SITE II or BINDING SITE III. Table 2 illustrates the complementarity of the nucleotide sequences of LOC256307 BINDING SITE, designated SEQ ID:3726, to the nucleotide sequence of VGAM212 RNA, herein designated VGAM RNA, also designated SEQ ID:547.

Another function of VGAM212 is therefore inhibition of LOC256307 (Accession XM_{—}173118). Accordingly, utilities of VGAM212 include diagnosis, prevention and treatment of diseases and clinical conditions associated with LOC256307. LOC87769 (Accession XM_{—}049058) is another VGAM212 host target gene. LOC87769 BINDING SITE is HOST TARGET binding site found in the 3′ untranslated region of mRNA encoded by LOC87769, corresponding to a HOST TARGET binding site such as BINDING SITE I, BINDING SITE II or BINDING SITE III. Table 2 illustrates the complementarity of the nucleotide sequences of LOC87769 BINDING SITE, designated SEQ ID:2912, to the nucleotide sequence of VGAM212 RNA, herein designated VGAM RNA, also designated SEQ ID:547.

Another function of VGAM212 is therefore inhibition of LOC87769 (Accession XM_{—}049058). Accordingly, utilities of VGAM212 include diagnosis, prevention and treatment of diseases and clinical conditions associated with LOC87769. LOC89890 (Accession XM_{—}026976) is another VGAM212 host target gene. LOC89890 BINDING SITE is HOST TARGET binding site found in the 3′ untranslated region of mRNA encoded by LOC89890, corresponding to a HOST TARGET binding site such as BINDING SITE I, BINDING SITE II or BINDING SITE III. Table 2 illustrates the complementarity of the nucleotide sequences of LOC89890 BINDING SITE, designated SEQ ID:2574, to the nucleotide sequence of VGAM212 RNA, herein designated VGAM RNA, also designated SEQ ID:547.

Another function of VGAM212 is therefore inhibition of LOC89890 (Accession XM_{—}026976). Accordingly, utilities of VGAM212 include diagnosis, prevention and treatment of diseases and clinical conditions associated with LOC89890. FIG. 1 further provides a conceptual description of a novel bioinformatically detected viral gene of the present invention, referred to here as Viral Genomic Address Messenger 213 (VGAM213) viral gene, which modulates expression of respective host target genes thereof, the function and utility of which host target genes is known in the art.

VGAM213 is a novel bioinformatically detected regulatory, non protein coding, viral micro RNA (miRNA) gene. The method by which VGAM213 was detected is described hereinabove with reference to FIGS. 1-8.

VGAM213 gene, herein designated VGAM GENE, is a viral gene contained in the genome of Vaccinia Virus. VGAM213 host target gene, herein designated VGAM HOST TARGET GENE, is a human gene contained in the human genome.

VGAM213 gene encodes a VGAM213 precursor RNA, herein designated VGAM PRECURSOR RNA. Similar to other miRNA genes, and unlike most ordinary genes, VGAM213 precursor RNA does not encode a protein. A nucleotide sequence identical or highly similar to the nucleotide sequence of VGAM213 precursor RNA is designated SEQ ID:199, and is provided hereinbelow with reference to the sequence listing part. Nucleotide sequence SEQ ID:199 is located at position 112175 relative to the genome of Vaccinia Virus.

VGAM213 precursor RNA folds onto itself, forming VGAM213 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM213 folded precursor RNA into VGAM213 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM213 RNA is designated SEQ ID:548, and is provided hereinbelow with reference to the sequence listing part.

VGAM213 host target gene, herein designated VGAM HOST TARGET GENE, encodes a corresponding messenger RNA, VGAM213 host target RNA, herein designated VGAM HOST TARGET RNA. VGAM213 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM213 RNA, herein designated VGAM RNA, binds complementarily to one or more host target binding sites located in untranslated regions of VGAM213 host target RNA, herein designated VGAM HOST TARGET RNA. This complementary binding is due to the fact that the nucleotide sequence of VGAM213 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM213 RNA, herein designated VGAM RNA, may have a different number of host target binding sites in untranslated regions of a VGAM213 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM213 RNA, herein designated VGAM RNA, to host target binding sites on VGAM213 host target RNA, herein designated VGAM HOST TARGET RNA, such as BINDING SITE I, BINDING SITE II and BINDING SITE III, inhibits translation of VGAM213 host target RNA into VGAM213 host target protein, herein designated VGAM HOST TARGET PROTEIN. VGAM host target protein is therefore outlined by a broken line.

It is appreciated that VGAM213 host target gene, herein designated VGAM HOST TARGET GENE, in fact represents a plurality of VGAM213 host target genes. The mRNA of each one of this plurality of VGAM213 host target genes comprises one or more host target binding sites, each having a nucleotide sequence which is at least partly complementary to VGAM213 RNA, herein designated VGAM RNA, and which when bound by VGAM213 RNA causes inhibition of translation of respective one or more VGAM213 host target proteins.

It is further appreciated by one skilled in the art that the mode of translational inhibition illustrated by FIG. 1 with specific reference to translational inhibition exerted by VGAM213 gene, herein designated VGAM GENE, on one or more VGAM213 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM213 is inhibition of expression of host target genes, as part of a novel viral mechanism of attacking a host. Accordingly, utilities of VGAM213 include diagnosis, prevention and treatment of viral infection by Vaccinia Virus. Specific functions, and accordingly utilities, of VGAM213 correlate with, and may be deduced from, the identity of the host target genes which VGAM213 binds and inhibits, and the function of these host target genes, as elaborated hereinbelow.

Nucleotide sequences of the VGAM213 precursor RNA, herein designated VGAM PRECURSOR RNA, and of the ‘diced’ VGAM213 RNA, herein designated VGAM RNA, and a schematic representation of the secondary folding of VGAM213 folded precursor RNA, herein designated VGAM FOLDED PRECURSOR RNA, of VGAM213 are further described hereinbelow with reference to Table 1.

Nucleotide sequences of host target binding sites, such as BINDING SITE-I, BINDING SITE-II and BINDING SITE-III of FIG. 1, found on VGAM213 host target RNA, and schematic representation of the complementarity of each of these host target binding sites to VGAM213 RNA, herein designated VGAM RNA, are described hereinbelow with reference to Table 2.

As mentioned hereinabove with reference to FIG. 1, a function of VGAM213 gene, herein designated VGAM is inhibition of expression of VGAM213 target genes. It is appreciated that specific functions, and accordingly utilities, of VGAM213 correlate with, and may be deduced from, the identity of the target genes which VGAM213 binds and inhibits, and the function of these target genes, as elaborated hereinbelow.

Growth Differentiation Factor 8 (GDF8, Accession NM_{—}005259) is a VGAM213 host target gene. GDF8 BINDING SITE is HOST TARGET binding site found in the 5′ untranslated region of mRNA encoded by GDF8, corresponding to a HOST TARGET binding site such as BINDING SITE I, BINDING SITE II or BINDING SITE III. Table 2 illustrates the complementarity of the nucleotide sequences of GDF8 BINDING SITE, designated SEQ ID:1196, to the nucleotide sequence of VGAM213 RNA, herein designated VGAM RNA, also designated SEQ ID:548.

A function of VGAM213 is therefore inhibition of Growth Differentiation Factor 8 (GDF8, Accession NM_{—}005259), a gene which acts specifically as a negative regulator of skeletal muscle growth. Accordingly, utilities of VGAM213 include diagnosis, prevention and treatment of diseases and clinical conditions associated with GDF8. The function of GDF8 has been established by previous studies. The transforming growth factor-beta super family encompasses a large number of growth and differentiation factors that play important roles in regulating embryonic development and in maintaining tissue homeostasis in adult animals. GDF8, or myostatin, is a member of this super family with a role in the control and maintenance of skeletal muscle mass. Animal model experiments lend further support to the function of GDF8. To determine the biologic function of Gdf8, McPherron et al. (1997) disrupted the Gdf8 gene by gene targeting in mice. Gdf8-null animals were significantly larger than wildtype animals and showed a large and widespread increase in skeletal muscle mass. Individual muscles of mutant animals weighed 2 to 3 times more than those of wildtype animals, and the increase in mass appeared to result from a combination of muscle cell hyperplasia and hypertrophy. McPherron et al. (1997) suggested that Gdf8 functions specifically as a negative regulator of skeletal muscle growth. Lin et al. (2002) observed increased skeletal muscle mass in their myostatin-null mouse model compared to wildtype animals as early as 4 weeks of age. In addition, the mutant mice showed reduced production and secretion of leptin (OMIM Ref. No. 164160) which was associated with reduced fat deposition. The reduced adipogenesis in the knockout mice suggested that myostatin is involved in regulating adiposity as well as muscularity.

It is appreciated that the abovementioned animal model for GDF8 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Gonzalez-Cadavid, N. F.; Taylor, W. E.; Yarasheski, K.; Sinha-Hikim, I.; Ma, K.; Ezzat, S.; Shen, R.; Lalani, R.; Asa, S.; Mamita, M.; Nair, G.; Arver, S.; Bhasin, S.: Organization of the human myostatin gene and expression in healthy men and HIV-infected men with muscle wasting. Proc. Nat. Acad. Sci. 95: 14938-14943, 1998.; and
Lin, J.; Arnold, H. B.; Della-Fera, M. A.; Azain, M. J.; Hartzell, D. L.; Baile, C. A.: Myostatin knockout in mice increases myogenesis and decreases adipogenesis. Biochem. Biophys. Res.

Further studies establishing the function and utilities of GDF8 are found in John Hopkins OMIM database record ID 601788, and in sited publications numbered 313-316, 2002-2003, 94 and 2050-1341 listed in the bibliography section hereinbelow, which are also hereby incorporated by reference. Regenerating Islet-derived-like, Pancreatic Stone Protein-like, Pancreatic Thread Protein-like (rat) (REGL, Accession NM_{—}006508) is another VGAM213 host target gene. REGL BINDING SITE is HOST TARGET binding site found in the 3′ untranslated region of mRNA encoded by REGL, corresponding to a HOST TARGET binding site such as BINDING SITE I, BINDING SITE II or BINDING SITE III. Table 2 illustrates the complementarity of the nucleotide sequences of REGL BINDING SITE, designated SEQ ID:1306, to the nucleotide sequence of VGAM213 RNA, herein designated VGAM RNA, also designated SEQ ID:548.

Another function of VGAM213 is therefore inhibition of Regenerating Islet-derived-like, Pancreatic Stone Protein-like, Pancreatic Thread Protein-like (rat) (REGL, Accession NM_{—}006508), a gene which is a member of REG family with unknown function. Accordingly, utilities of VGAM213 include diagnosis, prevention and treatment of diseases and clinical conditions associated with REGL. The function of REGL has been established by previous studies. The REG1A (OMIM Ref. No. 167770) and REG1B genes belong to the type I subclass of the REG family of genes, each of which encodes a 166-amino acid protein. Moriizumi et al. (1994) and Gharib et al. (1993) mapped the REG1A and REG1B genes to 2p12. Miyashita et al. (1995) demonstrated that 4 REG family genes are tandemly ordered in a 95-kb DNA region of 2p12. From analysis of YAC clones containing the 4 genes using 2-color fluorescence in situ hybridization, they demonstrated the following order: 2cen—PAP—RS—REG1A—REG1B—ptel. (RS, so designated for REG-related sequence, shows a high degree of homology to the REG1 genes but has an in-frame stop codon in the protein coding region.)

Full details of the abovementioned studies are described in the following publications, the disclosure of which are hereby incorporated by reference:
Miyashita, H.; Nakagawara, K.; Mori, M.; Narushima, Y.; Noguchi, N.; Moriizumi, S.; Takasawa, S.; Yonekura, H.; Takeuchi, T.; Okamoto, H.: Human REG family genes are tandemly ordered in a 95-kilobase region of chromosome 2p12. FEBS Lett. 377: 429-433, 1995.; and
Moriizumi, S.; Watanabe, T.; Unno, M.; Nakagawara, K.; Suzuki, Y.; Miyashita, H.; Yonekura, H.; Okamoto, H.: Isolation, structural determination and expression of a novel reg gene, hum.

Further studies establishing the function and utilities of REGL are found in John Hopkins OMIM database record ID 167771, and in sited publications numbered 2513-2515 listed in the bibliography section hereinbelow, which are also hereby incorporated by reference. DKFZP434J214 (Accession XM_{—}027639) is another VGAM213 host target gene. DKFZP434J214 BINDING SITE is HOST TARGET binding site found in the 3′ untranslated region of mRNA encoded by DKFZP434J214, corresponding to a HOST TARGET binding site such as BINDING SITE I, BINDING SITE II or BINDING SITE III. Table 2 illustrates the complementarity of the nucleotide sequences of DKFZP434J214 BINDING SITE, designated SEQ ID:2586, to the nucleotide sequence of VGAM213 RNA, herein designated VGAM RNA, also designated SEQ ID:548.

Another function of VGAM213 is therefore inhibition of DKFZP434J214 (Accession XM_{—}027639). Accordingly, utilities of VGAM213 include diagnosis, prevention and treatment of diseases and clinical conditions associated with DKFZP434J214. KIAA0440 (Accession NM_{—}015556) is another VGAM213 host target gene. KIAA0440 BINDING SITE is HOST TARGET binding site found in the 5′ untranslated region of mRNA encoded by KIAA0440, corresponding to a HOST TARGET binding site such as BINDING SITE I, BINDING SITE II or BINDING SITE III. Table 2 illustrates the complementarity of the nucleotide sequences of KIAA0440 BINDING SITE, designated SEQ ID: 1636, to the nucleotide sequence of VGAM213 RNA, herein designated VGAM RNA, also designated SEQ ID:548.

Another function of VGAM213 is therefore inhibition of KIAA0440 (Accession NM_{—}015556). Accordingly, utilities of VGAM213 include diagnosis, prevention and treatment of diseases and clinical conditions associated with KIAA0440. LOC152756 (Accession XM_{—}098262) is another VGAM213 host target gene. LOC152756 BINDING SITE is HOST TARGET binding site found in the 3′ untranslated region of mRNA encoded by LOC152756, corresponding to a HOST TARGET binding site such as BINDING SITE I, BINDING SITE II or BINDING SITE III. Table 2 illustrates the complementarity of the nucleotide sequences of LOC152756 BINDING SITE, designated SEQ ID:3363, to the nucleotide sequence of VGAM213 RNA, herein designated VGAM RNA, also designated SEQ ID:548.

Another function of VGAM213 is therefore inhibition of LOC152756 (Accession XM_{—}098262). Accordingly, utilities of VGAM213 include diagnosis, prevention and treatment of diseases and clinical conditions associated with LOC152756. LOC158428 (Accession XM_{—}047249) is another VGAM213 host target gene. LOC158428 BINDING SITE is HOST TARGET binding site found in the 3′ untranslated region of mRNA encoded by LOC158428, corresponding to a HOST TARGET binding site such as BINDING SITE I, BINDING SITE II or BINDING SITE III. Table 2 illustrates the complementarity of the nucleotide sequences of LOC158428 BINDING SITE, designated SEQ ID:2889, to the nucleotide sequence of VGAM213 RNA, herein designated VGAM RNA, also designated SEQ ID:548.

Another function of VGAM213 is therefore inhibition of LOC158428 (Accession XM_{—}047249). Accordingly, utilities of VGAM213 include diagnosis, prevention and treatment of diseases and clinical conditions associated with LOC158428. FIG. 1 further provides a conceptual description of a novel bioinformatically detected viral gene of the present invention, referred to here as Viral Genomic Address Messenger 214 (VGAM214) viral gene, which modulates expression of respective host target genes thereof, the function and utility of which host target genes is known in the art.

VGAM214 is a novel bioinformatically detected regulatory, non protein coding, viral micro RNA (miRNA) gene. The method by which VGAM214 was detected is described hereinabove with reference to FIGS. 1-8.

VGAM214 gene, herein designated VGAM GENE, is a viral gene contained in the genome of Vaccinia Virus. VGAM214 host target gene, herein designated VGAM HOST TARGET GENE, is a human gene contained in the human genome.

VGAM214 gene encodes a VGAM214 precursor RNA, herein designated VGAM PRECURSOR RNA. Similar to other miRNA genes, and unlike most ordinary genes, VGAM214 precursor RNA does not encode a protein. A nucleotide sequence identical or highly similar to the nucleotide sequence of VGAM214 precursor RNA is designated SEQ ID:200, and is provided hereinbelow with reference to the sequence listing part. Nucleotide sequence SEQ ID:200 is located at position 114150 relative to the genome of Vaccinia Virus.

VGAM214 precursor RNA folds onto itself, forming VGAM214 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM214 folded precursor RNA into VGAM214 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM214 RNA is designated SEQ ID:549, and is provided hereinbelow with reference to the sequence listing part.

VGAM214 host target gene, herein designated VGAM HOST TARGET GENE, encodes a corresponding messenger RNA, VGAM214 host target RNA, herein designated VGAM HOST TARGET RNA. VGAM214 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM214 RNA, herein designated VGAM RNA, binds complementarily to one or more host target binding sites located in untranslated regions of VGAM214 host target RNA, herein designated VGAM HOST TARGET RNA. This complementary binding is due to the fact that the nucleotide sequence of VGAM214 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM214 RNA, herein designated VGAM RNA, may have a different number of host target binding sites in untranslated regions of a VGAM214 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM214 RNA, herein designated VGAM RNA, to host target binding sites on VGAM214 host target RNA, herein designated VGAM HOST TARGET RNA, such as BINDING SITE I, BINDING SITE II and BINDING SITE III, inhibits translation of VGAM214 host target RNA into VGAM214 host target protein, herein designated VGAM HOST TARGET PROTEIN. VGAM host target protein is therefore outlined by a broken line.

It is appreciated that VGAM214 host target gene, herein designated VGAM HOST TARGET GENE, in fact represents a plurality of VGAM214 host target genes. The mRNA of each one of this plurality of VGAM214 host target genes comprises one or more host target binding sites, each having a nucleotide sequence which is at least partly complementary to VGAM214 RNA, herein designated VGAM RNA, and which when bound by VGAM214 RNA causes inhibition of translation of respective one or more VGAM214 host target proteins.

It is further appreciated by one skilled in the art that the mode of translational inhibition illustrated by FIG. 1 with specific reference to translational inhibition exerted by VGAM214 gene, herein designated VGAM GENE, on one or more VGAM214 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM214 is inhibition of expression of host target genes, as part of a novel viral mechanism of attacking a host. Accordingly, utilities of VGAM214 include diagnosis, prevention and treatment of viral infection by Vaccinia Virus. Specific functions, and accordingly utilities, of VGAM214 correlate with, and may be deduced from, the identity of the host target genes which VGAM214 binds and inhibits, and the function of these host target genes, as elaborated hereinbelow.

Nucleotide sequences of the VGAM214 precursor RNA, herein designated VGAM PRECURSOR RNA, and of the ‘diced’ VGAM214 RNA, herein designated VGAM RNA, and a schematic representation of the secondary folding of VGAM214 folded precursor RNA, herein designated VGAM FOLDED PRECURSOR RNA, of VGAM214 are further described hereinbelow with reference to Table 1.

Nucleotide sequences of host target binding sites, such as BINDING SITE-I, BINDING SITE-II and BINDING SITE-III of FIG. 1, found on VGAM214 host target RNA, and schematic representation of the complementarity of each of these host target binding sites to VGAM214 RNA, herein designated VGAM RNA, are described hereinbelow with reference to Table 2.

As mentioned hereinabove with reference to FIG. 1, a function of VGAM214 gene, herein designated VGAM is inhibition of expression of VGAM214 target genes. It is appreciated that specific functions, and accordingly utilities, of VGAM214 correlate with, and may be deduced from, the identity of the target genes which VGAM214 binds and inhibits, and the function of these target genes, as elaborated hereinbelow.

Basic Helix-loop-helix Domain Containing, Class B, 3 (BHLHB3, Accession NM_{—}030762) is a VGAM214 host target gene. BHLHB3 BINDING SITE is HOST TARGET binding site found in the 3′ untranslated region of mRNA encoded by BHLHB3, corresponding to a HOST TARGET binding site such as BINDING SITE I, BINDING SITE II or BINDING SITE III. Table 2 illustrates the complementarity of the nucleotide sequences of BHLHB3 BINDING SITE, designated SEQ ID:2162, to the nucleotide sequence of VGAM214 RNA, herein designated VGAM RNA, also designated SEQ ID:549.

A function of VGAM214 is therefore inhibition of Basic Helix-loop-helix Domain Containing, Class B, 3 (BHLHB3, Accession NM_{—}030762), a gene which represses both basal and activated transcription. Accordingly, utilities of VGAM214 include diagnosis, prevention and treatment of diseases and clinical conditions associated with BHLHB3. The function of BHLHB3 and its association with various diseases and clinical conditions, has been established by previous studies, as described hereinabove with reference to VGAM127. Coagulation Factor IX (plasma thromboplastic component, Christmas disease, hemophilia B) (F9, Accession NM_{—}000133) is another VGAM214 host target gene. F9 BINDING SITE is HOST TARGET binding site found in the 3′ untranslated region of mRNA encoded by F9, corresponding to a HOST TARGET binding site such as BINDING SITE I, BINDING SITE II or BINDING SITE III. Table 2 illustrates the complementarity of the nucleotide sequences of F9 BINDING SITE, designated SEQ ID:709, to the nucleotide sequence of VGAM214 RNA, herein designated VGAM RNA, also designated SEQ ID:549.

Another function of VGAM214 is therefore inhibition of Coagulation Factor IX (plasma thromboplastic component, Christmas disease, hemophilia B) (F9, Accession NM_{—}000133). Accordingly, utilities of VGAM214 include diagnosis, prevention and treatment of diseases and clinical conditions associated with F9. FLJ31737 (Accession NM_{—}144984) is another VGAM214 host target gene. FLJ31737 BINDING SITE is HOST TARGET binding site found in the 3′ untranslated region of mRNA encoded by FLJ31737, corresponding to a HOST TARGET binding site such as BINDING SITE I, BINDING SITE II or BINDING SITE III. Table 2 illustrates the complementarity of the nucleotide sequences of FLJ31737 BINDING SITE, designated SEQ ID:2504, to the nucleotide sequence of VGAM214 RNA, herein designated VGAM RNA, also designated SEQ ID:549.

Another function of VGAM214 is therefore inhibition of FLJ31737 (Accession NM_{—}144984). Accordingly, utilities of VGAM214 include diagnosis, prevention and treatment of diseases and clinical conditions associated with FLJ31737. KIAA1505 (Accession XM_{—}168469) is another VGAM214 host target gene. KIAA1505 BINDING SITE is HOST TARGET binding site found in the 5′ untranslated region of mRNA encoded by KIAA1505, corresponding to a HOST TARGET binding site such as BINDING SITE I, BINDING SITE II or BINDING SITE III. Table 2 illustrates the complementarity of the nucleotide sequences of KIAA1505 BINDING SITE, designated SEQ ID:3638, to the nucleotide sequence of VGAM214 RNA, herein designated VGAM RNA, also designated SEQ ID:549.

Another function of VGAM214 is therefore inhibition of KIAA1505 (Accession XM_{—}168469). Accordingly, utilities of VGAM214 include diagnosis, prevention and treatment of diseases and clinical conditions associated with KIAA1505. PHRET1 (Accession NM_{—}021200) is another VGAM214 host target gene. PHRET1 BINDING SITE is HOST TARGET binding site found in the 3′ untranslated region of mRNA encoded by PHRET1, corresponding to a HOST TARGET binding site such as BINDING SITE I, BINDING SITE II or BINDING SITE III. Table 2 illustrates the complementarity of the nucleotide sequences of PHRET1 BINDING SITE, designated SEQ ID:1947, to the nucleotide sequence of VGAM214 RNA, herein designated VGAM RNA, also designated SEQ ID:549.

Another function of VGAM214 is therefore inhibition of PHRET1 (Accession NM_{—}021200). Accordingly, utilities of VGAM214 include diagnosis, prevention and treatment of diseases and clinical conditions associated with PHRET1. RYD5 (Accession XM_{—}085737) is another VGAM214 host target gene. RYD5 BINDING SITE is HOST TARGET binding site found in the 3′ untranslated region of mRNA encoded by RYD5, corresponding to a HOST TARGET binding site such as BINDING SITE I, BINDING SITE II or BINDING SITE III. Table 2 illustrates the complementarity of the nucleotide sequences of RYD5 BINDING SITE, designated SEQ ID:3103, to the nucleotide sequence of VGAM214 RNA, herein designated VGAM RNA, also designated SEQ ID:549.

Another function of VGAM214 is therefore inhibition of RYD5 (Accession XM_{—}085737). Accordingly, utilities of VGAM214 include diagnosis, prevention and treatment of diseases and clinical conditions associated with RYD5. LOC221337 (Accession XM_{—}166387) is another VGAM214 host target gene. LOC221337 BINDING SITE is HOST TARGET binding site found in the 5′ untranslated region of mRNA encoded by LOC221337, corresponding to a HOST TARGET binding site such as BINDING SITE I, BINDING SITE II or BINDING SITE III. Table 2 illustrates the complementarity of the nucleotide sequences of LOC221337 BINDING SITE, designated SEQ ID:3556, to the nucleotide sequence of VGAM214 RNA, herein designated VGAM RNA, also designated SEQ ID:549.

Another function of VGAM214 is therefore inhibition of LOC221337 (Accession XM_{—}166387). Accordingly, utilities of VGAM214 include diagnosis, prevention and treatment of diseases and clinical conditions associated with LOC221337. FIG. 1 further provides a conceptual description of a novel bioinformatically detected viral gene of the present invention, referred to here as Viral Genomic Address Messenger 215 (VGAM215) viral gene, which modulates expression of respective host target genes thereof, the function and utility of which host target genes is known in the art.

VGAM215 is a novel bioinformatically detected regulatory, non protein coding, viral micro RNA (miRNA) gene. The method by which VGAM215 was detected is described hereinabove with reference to FIGS. 1-8.

VGAM215 gene, herein designated VGAM GENE, is a viral gene contained in the genome of Vaccinia Virus. VGAM215 host target gene, herein designated VGAM HOST TARGET GENE, is a human gene contained in the human genome.

VGAM215 gene encodes a VGAM215 precursor RNA, herein designated VGAM PRECURSOR RNA. Similar to other miRNA genes, and unlike most ordinary genes, VGAM215 precursor RNA does not encode a protein. A nucleotide sequence identical or highly similar to the nucleotide sequence of VGAM215 precursor RNA is designated SEQ ID:201, and is provided hereinbelow with reference to the sequence listing part. Nucleotide sequence SEQ ID:201 is located at position 113419 relative to the genome of Vaccinia Virus.

VGAM215 precursor RNA folds onto itself, forming VGAM215 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM215 folded precursor RNA into VGAM215 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM215 RNA is designated SEQ ID:550, and is provided hereinbelow with reference to the sequence listing part.

VGAM215 host target gene, herein designated VGAM HOST TARGET GENE, encodes a corresponding messenger RNA, VGAM215 host target RNA, herein designated VGAM HOST TARGET RNA. VGAM215 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM215 RNA, herein designated VGAM RNA, binds complementarily to one or more host target binding sites located in untranslated regions of VGAM215 host target RNA, herein designated VGAM HOST TARGET RNA. This complementary binding is due to the fact that the nucleotide sequence of VGAM215 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM215 RNA, herein designated VGAM RNA, may have a different number of host target binding sites in untranslated regions of a VGAM215 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM215 RNA, herein designated VGAM RNA, to host target binding sites on VGAM215 host target RNA, herein designated VGAM HOST TARGET RNA, such as BINDING SITE I, BINDING SITE II and BINDING SITE III, inhibits translation of VGAM215 host target RNA into VGAM215 host target protein, herein designated VGAM HOST TARGET PROTEIN. VGAM host target protein is therefore outlined by a broken line.

It is appreciated that VGAM215 host target gene, herein designated VGAM HOST TARGET GENE, in fact represents a plurality of VGAM215 host target genes. The mRNA of each one of this plurality of VGAM215 host target genes comprises one or more host target binding sites, each having a nucleotide sequence which is at least partly complementary to VGAM215 RNA, herein designated VGAM RNA, and which when bound by VGAM215 RNA causes inhibition of translation of respective one or more VGAM215 host target proteins.

It is further appreciated by one skilled in the art that the mode of translational inhibition illustrated by FIG. 1 with specific reference to translational inhibition exerted by VGAM215 gene, herein designated VGAM GENE, on one or more VGAM215 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM215 is inhibition of expression of host target genes, as part of a novel viral mechanism of attacking a host. Accordingly, utilities of VGAM215 include diagnosis, prevention and treatment of viral infection by Vaccinia Virus. Specific functions, and accordingly utilities, of VGAM215 correlate with, and may be deduced from, the identity of the host target genes which VGAM215 binds and inhibits, and the function of these host target genes, as elaborated hereinbelow.

Nucleotide sequences of the VGAM215 precursor RNA, herein designated VGAM PRECURSOR RNA, and of the ‘diced’ VGAM215 RNA, herein designated VGAM RNA, and a schematic representation of the secondary folding of VGAM215 folded precursor RNA, herein designated VGAM FOLDED PRECURSOR RNA, of VGAM215 are further described hereinbelow with reference to Table 1.

Nucleotide sequences of host target binding sites, such as BINDING SITE-I, BINDING SITE-II and BINDING SITE-III of FIG. 1, found on VGAM215 host target RNA, and schematic representation of the complementarity of each of these host target binding sites to VGAM215 RNA, herein designated VGAM RNA, are described hereinbelow with reference to Table 2.

As mentioned hereinabove with reference to FIG. 1, a function of VGAM215 gene, herein designated VGAM is inhibition of expression of VGAM215 target genes. It is appreciated that specific functions, and accordingly utilities, of VGAM215 correlate with, and may be deduced from, the identity of the target genes which VGAM215 binds and inhibits, and the function of these target genes, as elaborated hereinbelow.

Adenylate Cyclase 9 (ADCY9, Accession NM_{—}001116) is a VGAM215 host target gene. ADCY9 BINDING SITE is HOST TARGET binding site found in the 3′ untranslated region of mRNA encoded by ADCY9, corresponding to a HOST TARGET binding site such as BINDING SITE I, BINDING SITE II or BINDING SITE III. Table 2 illustrates the complementarity of the nucleotide sequences of ADCY9 BINDING SITE, designated SEQ ID:801, to the nucleotide sequence of VGAM215 RNA, herein designated VGAM RNA, also designated SEQ ID:550.

A function of VGAM215 is therefore inhibition of Adenylate Cyclase 9 (ADCY9, Accession NM_{—}001116), a gene which . may be a physiologically relevant docking site for calcineurin (by similarity). Accordingly, utilities of VGAM215 include diagnosis, prevention and treatment of diseases and clinical conditions associated with ADCY9. The function of ADCY9 has been established by previous studies. The adenylyl cyclases (EC 4.6.1.1) are membrane-associated enzymes that are expressed in most human tissues. These enzymes catalyze the formation of cAMP and are regulated by a family of G protein-coupled receptors, protein kinases, and calcium. The type 9 adenylyl cyclase (ADCY9) is a widely distributed adenylyl cyclase that was originally cloned from mouse (Paterson et al., 1995; Premont et al., 1996). Hacker et al. (1998) cloned human cardiac ADCY9, or AC9, cDNAs and found that the deduced 1,294-amino acid protein is 90% identical to mouse Adcy9. Like mouse Adcy9, the predicted human ADCY9 protein contains 12 transmembrane domains, Asn-linked glycosylation sites, and cAMP-dependent protein kinase phosphorylation sites; however, these proteins differ in the C2b domain due to a frameshift in the human ADCY9 coding sequence relative to the coding sequence of mouse Adcy9. Northern blot analysis detected 8.5- and 6.3-kb ADCY9 transcripts in all human tissues examined. By fluorescence in situ hybridization, Hacker et al. (1998) mapped the human and mouse ADCY9 genes to 16p13.3 and chromosome 16 band B1, respectively.

Full details of the abovementioned studies are described in the following publications, the disclosure of which are hereby incorporated by reference:
Premont, R. T.; Matsuoka, I.; Mattei, M. G.; Pouille, Y.; Defer, N.; Hanoune, J.: Identification and characterization of a widely expressed form of adenylyl cyclase. J. Biol. Chem. 271: 13900-13907, 1996.; and
Hacker, B. M.; Tomlinson, J. E.; Wayman, G. A.; Sultana, R.; Chan, G.; Villacres, E.; Disteche, C.; Storm, D. R.: Cloning, chromosomal mapping, and regulatory properties of the human ty.

Further studies establishing the function and utilities of ADCY9 are found in John Hopkins OMIM database record ID 603302, and in sited publications numbered 581-584 listed in the bibliography section hereinbelow, which are also hereby incorporated by reference. Carnitine Acetyltransferase (CRAT, Accession NM_{—}004003) is another VGAM215 host target gene. CRAT BINDING SITE1 and CRAT BINDING SITE2 are HOST TARGET binding sites found in untranslated regions of mRNA encoded by CRAT, corresponding to HOST TARGET binding sites such as BINDING SITE I, BINDING SITE II or BINDING SITE III. Table 2 illustrates the complementarity of the nucleotide sequences of CRAT BINDING SITE1 and CRAT BINDING SITE2, designated SEQ ID: 1079 and SEQ ID:771 respectively, to the nucleotide sequence of VGAM215 RNA, herein designated VGAM RNA, also designated SEQ ID:550.

Another function of VGAM215 is therefore inhibition of Carnitine Acetyltransferase (CRAT, Accession NM_{—}004003), a gene which catalyzes the reversible transfer of acyl groups from an acyl-CoA thioester to carnitine. Accordingly, utilities of VGAM215 include diagnosis, prevention and treatment of diseases and clinical conditions associated with CRAT. The function of CRAT has been established by previous studies. Carnitine acyltransferases are a group of enzymes that catalyze the reversible transfer of acyl groups from an acyl-CoA thioester to carnitine, thus forming the corresponding acylcarnitine. These enzymes can be distinguished according to their substrate specificity in carnitine palmitoyltransferase (see OMIM Ref. No. CPT1, 600528 and CPT2, 600650), carnitine octanoyl-transferase (CROT; 606090), and carnitine acetyltransferase (EC 2.3.1.7). CRAT is a key enzyme for metabolic pathways involved with the control of the acyl-CoA/CoA ratio in mitochondria, peroxisomes, and endoplasmic reticulum Acetylcarnitine, which can be a precursor for acetylcholine synthesis catalyzed by choline acetyltransferase, is thought to slow the rate of mental deterioration in Alzheimer patients, and Kalaria and Harik (1992) found decreased function of CRAT in the brain of Alzheimer patients

Full details of the abovementioned studies are described in the following publications, the disclosure of which are hereby incorporated by reference:
Kalaria, R. N.; Harik, S. I.: Carnitine acetyltransferase activity in the human brain and its microvessels is decreased in Alzheimer's disease. Ann. Neurol. 32: 583-586, 1992.; and
van der Leij, F. R.; Huijkman, N. C. A.; Boomsma, C.; Kuipers, J. R. G.; Bartelds, B.: Genomics of the human carnitine acyltransferase genes. Molec. Genet. Metab. 71: 139-153, 2000.

Further studies establishing the function and utilities of CRAT are found in John Hopkins OMIM database record ID 600184, and in sited publications numbered 1769-1771 listed in the bibliography section hereinbelow, which are also hereby incorporated by reference. Agmatine Ureohydrolase (agmatinase) (AGMAT, Accession NM_{—}024758) is another VGAM215 host target gene. AGMAT BINDING SITE is HOST TARGET binding site found in the 3′ untranslated region of mRNA encoded by AGMAT, corresponding to a HOST TARGET binding site such as BINDING SITE I, BINDING SITE II or BINDING SITE III. Table 2 illustrates the complementarity of the nucleotide sequences of AGMAT BINDING SITE, designated SEQ ID:2087, to the nucleotide sequence of VGAM215 RNA, herein designated VGAM RNA, also designated SEQ ID:550.

Another function of VGAM215 is therefore inhibition of Agmatine Ureohydrolase (agmatinase) (AGMAT, Accession NM_{—}024758). Accordingly, utilities of VGAM215 include diagnosis, prevention and treatment of diseases and clinical conditions associated with AGMAT. DKFZp434E0519 (Accession NM_{—}032247) is another VGAM215 host target gene. DKFZp434E0519 BINDING SITE is HOST TARGET binding site found in the 3′ untranslated region of mRNA encoded by DKFZp434E0519, corresponding to a HOST TARGET binding site such as BINDING SITE I, BINDING SITE II or BINDING SITE III. Table 2 illustrates the complementarity of the nucleotide sequences of DKFZp434E0519 BINDING SITE, designated SEQ ID:2240, to the nucleotide sequence of VGAM215 RNA, herein designated VGAM RNA, also designated SEQ ID:550.

Another function of VGAM215 is therefore inhibition of DKFZp434E0519 (Accession NM_{—}032247). Accordingly, utilities of VGAM215 include diagnosis, prevention and treatment of diseases and clinical conditions associated with DKFZp434E0519. DKFZp762E1312 (Accession NM_{—}018410) is another VGAM215 host target gene. DKFZp762E1312 BINDING SITE is HOST TARGET binding site found in the 5′ untranslated region of mRNA encoded by DKFZp762E1312, corresponding to a HOST TARGET binding site such as BINDING SITE I, BINDING SITE II or BINDING SITE III. Table 2 illustrates the complementarity of the nucleotide sequences of DKFZp762E1312 BINDING SITE, designated SEQ ID:1823, to the nucleotide sequence of VGAM215 RNA, herein designated VGAM RNA, also designated SEQ ID:550.

Another function of VGAM215 is therefore inhibition of DKFZp762E1312 (Accession NM_{—}018410). Accordingly, utilities of VGAM215 include diagnosis, prevention and treatment of diseases and clinical conditions associated with DKFZp762E1312. FLJ20619 (Accession NM_{—}017904) is another VGAM215 host target gene. FLJ20619 BINDING SITE is HOST TARGET binding site found in the 3′ untranslated region of mRNA encoded by FLJ20619, corresponding to a HOST TARGET binding site such as BINDING SITE I, BINDING SITE II or BINDING SITE III. Table 2 illustrates the complementarity of the nucleotide sequences of FLJ20619 BINDING SITE, designated SEQ ID:1760, to the nucleotide sequence of VGAM215 RNA, herein designated VGAM RNA, also designated SEQ ID:550.

Another function of VGAM215 is therefore inhibition of FLJ20619 (Accession NM_{—}017904). Accordingly, utilities of VGAM215 include diagnosis, prevention and treatment of diseases and clinical conditions associated with FLJ20619. FLJ20716 (Accession NM_{—}017938) is another VGAM215 host target gene. FLJ20716 BINDING SITE is HOST TARGET binding site found in the 3′ untranslated region of mRNA encoded by FLJ20716, corresponding to a HOST TARGET binding site such as BINDING SITE I, BINDING SITE II or BINDING SITE III. Table 2 illustrates the complementarity of the nucleotide sequences of FLJ20716 BINDING SITE, designated SEQ ID:1768, to the nucleotide sequence of VGAM215 RNA, herein designated VGAM RNA, also designated SEQ ID:550.

Another function of VGAM215 is therefore inhibition of FLJ20716 (Accession NM_{—}017938). Accordingly, utilities of VGAM215 include diagnosis, prevention and treatment of diseases and clinical conditions associated with FLJ20716. Frequently Rearranged In Advanced T-cell Lymphomas (FRAT1, Accession NM_{—}005479) is another VGAM215 host target gene. FRAT1 BINDING SITE is HOST TARGET binding site found in the 3′ untranslated region of mRNA encoded by FRAT1, corresponding to a HOST TARGET binding site such as BINDING SITE I, BINDING SITE II or BINDING SITE III. Table 2 illustrates the complementarity of the nucleotide sequences of FRAT1 BINDING SITE, designated SEQ ID:1217, to the nucleotide sequence of VGAM215 RNA, herein designated VGAM RNA, also designated SEQ ID:550.

Another function of VGAM215 is therefore inhibition of Frequently Rearranged In Advanced T-cell Lymphomas (FRAT1, Accession NM_{—}005479). Accordingly, utilities of VGAM215 include diagnosis, prevention and treatment of diseases and clinical conditions associated with FRAT1. KIAA1656 (Accession XM_{—}038022) is another VGAM215 host target gene. KIAA1656 BINDING SITE is HOST TARGET binding site found in the 3′ untranslated region of mRNA encoded by KIAA1656, corresponding to a HOST TARGET binding site such as BINDING SITE I, BINDING SITE II or BINDING SITE III. Table 2 illustrates the complementarity of the nucleotide sequences of KIAA1656 BINDING SITE, designated SEQ ID:2729, to the nucleotide sequence of VGAM215 RNA, herein designated VGAM RNA, also designated SEQ ID:550.

Another function of VGAM215 is therefore inhibition of KIAA1656 (Accession XM_{—}038022). Accordingly, utilities of VGAM215 include diagnosis, prevention and treatment of diseases and clinical conditions associated with KIAA1656. Rab11-FIP3 (Accession NM_{—}014700) is another VGAM215 host target gene. Rab11-FIP3 BINDING SITE is HOST TARGET binding site found in the 3′ untranslated region of mRNA encoded by Rab11-FIP3, corresponding to a HOST TARGET binding site such as BINDING SITE I, BINDING SITE II or BINDING SITE III. Table 2 illustrates the complementarity of the nucleotide sequences of Rab11-FIP3 BINDING SITE, designated SEQ ID:1524, to the nucleotide sequence of VGAM215 RNA, herein designated VGAM RNA, also designated SEQ ID:550.

Another function of VGAM215 is therefore inhibition of Rab11-FIP3 (Accession NM_{—}014700). Accordingly, utilities of VGAM215 include diagnosis, prevention and treatment of diseases and clinical conditions associated with Rab11-FIP3. Translocase of Outer Mitochondrial Membrane 34 (TOMM34, Accession NM_{—}006809) is another VGAM215 host target gene. TOMM34 BINDING SITE is HOST TARGET binding site found in the 3′ untranslated region of mRNA encoded by TOMM34, corresponding to a HOST TARGET binding site such as BINDING SITE I, BINDING SITE II or BINDING SITE III. Table 2 illustrates the complementarity of the nucleotide sequences of TOMM34 BINDING SITE, designated SEQ ID:1332, to the nucleotide sequence of VGAM215 RNA, herein designated VGAM RNA, also designated SEQ ID:550.

Another function of VGAM215 is therefore inhibition of Translocase of Outer Mitochondrial Membrane 34 (TOMM34, Accession NM_{—}006809). Accordingly, utilities of VGAM215 include diagnosis, prevention and treatment of diseases and clinical conditions associated with TOMM34. LOC146337 (Accession XM_{—}096982) is another VGAM215 host target gene. LOC146337 BINDING SITE is HOST TARGET binding site found in the 3′ untranslated region of mRNA encoded by LOC146337, corresponding to a HOST TARGET binding site such as BINDING SITE I, BINDING SITE II or BINDING SITE III. Table 2 illustrates the complementarity of the nucleotide sequences of LOC146337 BINDING SITE, designated SEQ ID:3285, to the nucleotide sequence of VGAM215 RNA, herein designated VGAM RNA, also designated SEQ ID:550.

Another function of VGAM215 is therefore inhibition of LOC146337 (Accession XM_{—}096982). Accordingly, utilities of VGAM215 include diagnosis, prevention and treatment of diseases and clinical conditions associated with LOC146337. LOC147229 (Accession XM_{—}085742) is another VGAM215 host target gene. LOC147229 BINDING SITE is HOST TARGET binding site found in the 3′ untranslated region of mRNA encoded by LOC147229, corresponding to a HOST TARGET binding site such as BINDING SITE I, BINDING SITE II or BINDING SITE III. Table 2 illustrates the complementarity of the nucleotide sequences of LOC147229 BINDING SITE, designated SEQ ID:3105, to the nucleotide sequence of VGAM215 RNA, herein designated VGAM RNA, also designated SEQ ID:550.

Another function of VGAM215 is therefore inhibition of LOC147229 (Accession XM_{—}085742). Accordingly, utilities of VGAM215 include diagnosis, prevention and treatment of diseases and clinical conditions associated with LOC147229. LOC158332 (Accession XM_{—}088554) is another VGAM215 host target gene. LOC158332 BINDING SITE is HOST TARGET binding site found in the 3′ untranslated region of mRNA encoded by LOC158332, corresponding to a HOST TARGET binding site such as BINDING SITE I, BINDING SITE II or BINDING SITE III. Table 2 illustrates the complementarity of the nucleotide sequences of LOC158332 BINDING SITE, designated SEQ ID:3217, to the nucleotide sequence of VGAM215 RNA, herein designated VGAM RNA, also designated SEQ ID:550.

Another function of VGAM215 is therefore inhibition of LOC158332 (Accession XM_{—}088554). Accordingly, utilities of VGAM215 include diagnosis, prevention and treatment of diseases and clinical conditions associated with LOC158332. LOC163131 (Accession XM_{—}092019) is another VGAM215 host target gene. LOC163131 BINDING SITE is HOST TARGET binding site found in the 5′ untranslated region of mRNA encoded by LOC163131, corresponding to a HOST TARGET binding site such as BINDING SITE I, BINDING SITE II or BINDING SITE III. Table 2 illustrates the complementarity of the nucleotide sequences of LOC163131 BINDING SITE, designated SEQ ID:3245, to the nucleotide sequence of VGAM215 RNA, herein designated VGAM RNA, also designated SEQ ID:550.

Another function of VGAM215 is therefore inhibition of LOC163131 (Accession XM_{—}092019). Accordingly, utilities of VGAM215 include diagnosis, prevention and treatment of diseases and clinical conditions associated with LOC163131. LOC221423 (Accession XM_{—}166362) is another VGAM215 host target gene. LOC221423 BINDING SITE is HOST TARGET binding site found in the 3′ untranslated region of mRNA encoded by LOC221423, corresponding to a HOST TARGET binding site such as BINDING SITE I, BINDING SITE II or BINDING SITE III. Table 2 illustrates the complementarity of the nucleotide sequences of LOC221423 BINDING SITE, designated SEQ ID:3551, to the nucleotide sequence of VGAM215 RNA, herein designated VGAM RNA, also designated SEQ ID:550.

Another function of VGAM215 is therefore inhibition of LOC221423 (Accession XM_{—}166362). Accordingly, utilities of VGAM215 include diagnosis, prevention and treatment of diseases and clinical conditions associated with LOC221423. LOC222182 (Accession XM_{—}168471) is another VGAM215 host target gene. LOC222182 BINDING SITE is HOST TARGET binding site found in the 5′ untranslated region of mRNA encoded by LOC222182, corresponding to a HOST TARGET binding site such as BINDING SITE I, BINDING SITE II or BINDING SITE III. Table 2 illustrates the complementarity of the nucleotide sequences of LOC222182 BINDING SITE, designated SEQ ID:3639, to the nucleotide sequence of VGAM215 RNA, herein designated VGAM RNA, also designated SEQ ID:550.

Another function of VGAM215 is therefore inhibition of LOC222182 (Accession XM_{—}168471). Accordingly, utilities of VGAM215 include diagnosis, prevention and treatment of diseases and clinical conditions associated with LOC222182. FIG. 1 further provides a conceptual description of a novel bioinformatically detected viral gene of the present invention, referred to here as Viral Genomic Address Messenger 216 (VGAM216) viral gene, which modulates expression of respective host target genes thereof, the function and utility of which host target genes is known in the art.

VGAM216 is a novel bioinformatically detected regulatory, non protein coding, viral micro RNA (miRNA) gene. The method by which VGAM216 was detected is described hereinabove with reference to FIGS. 1-8.

VGAM216 gene, herein designated VGAM GENE, is a viral gene contained in the genome of Vaccinia Virus. VGAM216 host target gene, herein designated VGAM HOST TARGET GENE, is a human gene contained in the human genome.

VGAM216 gene encodes a VGAM216 precursor RNA, herein designated VGAM PRECURSOR RNA. Similar to other miRNA genes, and unlike most ordinary genes, VGAM216 precursor RNA does not encode a protein. A nucleotide sequence identical or highly similar to the nucleotide sequence of VGAM216 precursor RNA is designated SEQ ID:202, and is provided hereinbelow with reference to the sequence listing part. Nucleotide sequence SEQ ID:202 is located at position 112722 relative to the genome of Vaccinia Virus.

VGAM216 precursor RNA folds onto itself, forming VGAM216 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM216 folded precursor RNA into VGAM216 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM216 RNA is designated SEQ ID:551, and is provided hereinbelow with reference to the sequence listing part.

VGAM216 host target gene, herein designated VGAM HOST TARGET GENE, encodes a corresponding messenger RNA, VGAM216 host target RNA, herein designated VGAM HOST TARGET RNA. VGAM216 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM216 RNA, herein designated VGAM RNA, binds complementarily to one or more host target binding sites located in untranslated regions of VGAM216 host target RNA, herein designated VGAM HOST TARGET RNA. This complementary binding is due to the fact that the nucleotide sequence of VGAM216 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM216 RNA, herein designated VGAM RNA, may have a different number of host target binding sites in untranslated regions of a VGAM216 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM216 RNA, herein designated VGAM RNA, to host target binding sites on VGAM216 host target RNA, herein designated VGAM HOST TARGET RNA, such as BINDING SITE I, BINDING SITE II and BINDING SITE III, inhibits translation of VGAM216 host target RNA into VGAM216 host target protein, herein designated VGAM HOST TARGET PROTEIN. VGAM host target protein is therefore outlined by a broken line.

It is appreciated that VGAM216 host target gene, herein designated VGAM HOST TARGET GENE, in fact represents a plurality of VGAM216 host target genes. The mRNA of each one of this plurality of VGAM216 host target genes comprises one or more host target binding sites, each having a nucleotide sequence which is at least partly complementary to VGAM216 RNA, herein designated VGAM RNA, and which when bound by VGAM216 RNA causes inhibition of translation of respective one or more VGAM216 host target proteins.

It is further appreciated by one skilled in the art that the mode of translational inhibition illustrated by FIG. 1 with specific reference to translational inhibition exerted by VGAM216 gene, herein designated VGAM GENE, on one or more VGAM216 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM216 is inhibition of expression of host target genes, as part of a novel viral mechanism of attacking a host. Accordingly, utilities of VGAM216 include diagnosis, prevention and treatment of viral infection by Vaccinia Virus. Specific functions, and accordingly utilities, of VGAM216 correlate with, and may be deduced from, the identity of the host target genes which VGAM216 binds and inhibits, and the function of these host target genes, as elaborated hereinbelow.

Nucleotide sequences of the VGAM216 precursor RNA, herein designated VGAM PRECURSOR RNA, and of the ‘diced’ VGAM216 RNA, herein designated VGAM RNA, and a schematic representation of the secondary folding of VGAM216 folded precursor RNA, herein designated VGAM FOLDED PRECURSOR RNA, of VGAM216 are further described hereinbelow with reference to Table 1.

Nucleotide sequences of host target binding sites, such as BINDING SITE-I, BINDING SITE-II and BINDING SITE-III of FIG. 1, found on VGAM216 host target RNA, and schematic representation of the complementarity of each of these host target binding sites to VGAM216 RNA, herein designated VGAM RNA, are described hereinbelow with reference to Table 2.

As mentioned hereinabove with reference to FIG. 1, a function of VGAM216 gene, herein designated VGAM is inhibition of expression of VGAM216 target genes. It is appreciated that specific functions, and accordingly utilities, of VGAM216 correlate with, and may be deduced from, the identity of the target genes which VGAM216 binds and inhibits, and the function of these target genes, as elaborated hereinbelow.

UDP-Gal:betaGlcNAc Beta 1,3-galactosyltransferase, Polypeptide 3 (B3GALT3, Accession NM_{—}003781) is a VGAM216 host target gene. B3GALT3 BINDING SITE is HOST TARGET binding site found in the 3′ untranslated region of mRNA encoded by B3GALT3, corresponding to a HOST TARGET binding site such as BINDING SITE I, BINDING SITE II or BINDING SITE III. Table 2 illustrates the complementarity of the nucleotide sequences of B3GALT3 BINDING SITE, designated SEQ ID: 1056, to the nucleotide sequence of VGAM216 RNA, herein designated VGAM RNA, also designated SEQ ID:551.

A function of VGAM216 is therefore inhibition of UDP-Gal:betaGlcNAc Beta 1,3-galactosyltransferase, Polypeptide 3 (B3GALT3, Accession NM_{—}003781). Accordingly, utilities of VGAM216 include diagnosis, prevention and treatment of diseases and clinical conditions associated with B3GALT3. LOC159199 (Accession XM_{—}089441) is another VGAM216 host target gene. LOC159199 BINDING SITE is HOST TARGET binding site found in the 5′ untranslated region of mRNA encoded by LOC159199, corresponding to a HOST TARGET binding site such as BINDING SITE I, BINDING SITE II or BINDING SITE III. Table 2 illustrates the complementarity of the nucleotide sequences of LOC159199 BINDING SITE, designated SEQ ID:3237, to the nucleotide sequence of VGAM216 RNA, herein designated VGAM RNA, also designated SEQ ID:551.

Another function of VGAM216 is therefore inhibition of LOC159199 (Accession XM_{—}089441). Accordingly, utilities of VGAM216 include diagnosis, prevention and treatment of diseases and clinical conditions associated with LOC159199. FIG. 1 further provides a conceptual description of a novel bioinformatically detected viral gene of the present invention, referred to here as Viral Genomic Address Messenger 217 (VGAM217) viral gene, which modulates expression of respective host target genes thereof, the function and utility of which host target genes is known in the art.

VGAM217 is a novel bioinformatically detected regulatory, non protein coding, viral micro RNA (miRNA) gene. The method by which VGAM217 was detected is described hereinabove with reference to FIGS. 1-8.

VGAM217 gene, herein designated VGAM GENE, is a viral gene contained in the genome of Vaccinia Virus. VGAM217 host target gene, herein designated VGAM HOST TARGET GENE, is a human gene contained in the human genome.

VGAM217 gene encodes a VGAM217 precursor RNA, herein designated VGAM PRECURSOR RNA. Similar to other miRNA genes, and unlike most ordinary genes, VGAM217 precursor RNA does not encode a protein. A nucleotide sequence identical or highly similar to the nucleotide sequence of VGAM217 precursor RNA is designated SEQ ID:203, and is provided hereinbelow with reference to the sequence listing part. Nucleotide sequence SEQ ID:203 is located at position 113751 relative to the genome of Vaccinia Virus.

VGAM217 precursor RNA folds onto itself, forming VGAM217 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM217 folded precursor RNA into VGAM217 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM217 RNA is designated SEQ ID:552, and is provided hereinbelow with reference to the sequence listing part.

VGAM217 host target gene, herein designated VGAM HOST TARGET GENE, encodes a corresponding messenger RNA, VGAM217 host target RNA, herein designated VGAM HOST TARGET RNA. VGAM217 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM217 RNA, herein designated VGAM RNA, binds complementarily to one or more host target binding sites located in untranslated regions of VGAM217 host target RNA, herein designated VGAM HOST TARGET RNA. This complementary binding is due to the fact that the nucleotide sequence of VGAM217 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM217 RNA, herein designated VGAM RNA, may have a different number of host target binding sites in untranslated regions of a VGAM217 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM217 RNA, herein designated VGAM RNA, to host target binding sites on VGAM217 host target RNA, herein designated VGAM HOST TARGET RNA, such as BINDING SITE I, BINDING SITE II and BINDING SITE III, inhibits translation of VGAM217 host target RNA into VGAM217 host target protein, herein designated VGAM HOST TARGET PROTEIN. VGAM host target protein is therefore outlined by a broken line.

It is appreciated that VGAM217 host target gene, herein designated VGAM HOST TARGET GENE, in fact represents a plurality of VGAM217 host target genes. The mRNA of each one of this plurality of VGAM217 host target genes comprises one or more host target binding sites, each having a nucleotide sequence which is at least partly complementary to VGAM217 RNA, herein designated VGAM RNA, and which when bound by VGAM217 RNA causes inhibition of translation of respective one or more VGAM217 host target proteins.

It is further appreciated by one skilled in the art that the mode of translational inhibition illustrated by FIG. 1 with specific reference to translational inhibition exerted by VGAM217 gene, herein designated VGAM GENE, on one or more VGAM217 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM217 is inhibition of expression of host target genes, as part of a novel viral mechanism of attacking a host. Accordingly, utilities of VGAM217 include diagnosis, prevention and treatment of viral infection by Vaccinia Virus. Specific functions, and accordingly utilities, of VGAM217 correlate with, and may be deduced from, the identity of the host target genes which VGAM217 binds and inhibits, and the function of these host target genes, as elaborated hereinbelow.

Nucleotide sequences of the VGAM217 precursor RNA, herein designated VGAM PRECURSOR RNA, and of the ‘diced’ VGAM217 RNA, herein designated VGAM RNA, and a schematic representation of the secondary folding of VGAM217 folded precursor RNA, herein designated VGAM FOLDED PRECURSOR RNA, of VGAM217 are further described hereinbelow with reference to Table 1.

Nucleotide sequences of host target binding sites, such as BINDING SITE-I, BINDING SITE-II and BINDING SITE-III of FIG. 1, found on VGAM217 host target RNA, and schematic representation of the complementarity of each of these host target binding sites to VGAM217 RNA, herein designated VGAM RNA, are described hereinbelow with reference to Table 2.

As mentioned hereinabove with reference to FIG. 1, a function of VGAM217 gene, herein designated VGAM is inhibition of expression of VGAM217 target genes. It is appreciated that specific functions, and accordingly utilities, of VGAM217 correlate with, and may be deduced from, the identity of the target genes which VGAM217 binds and inhibits, and the function of these target genes, as elaborated hereinbelow.

Amiloride-sensitive Cation Channel 2, Neuronal (ACCN2, Accession NM_{—}020039) is a VGAM217 host target gene. ACCN2 BINDING SITE is HOST TARGET binding site found in the 3′ untranslated region of mRNA encoded by ACCN2, corresponding to a HOST TARGET binding site such as BINDING SITE I, BINDING SITE II or BINDING SITE III. Table 2 illustrates the complementarity of the nucleotide sequences of ACCN2 BINDING SITE, designated SEQ ID:1891, to the nucleotide sequence of VGAM217 RNA, herein designated VGAM RNA, also designated SEQ ID:552.

A function of VGAM217 is therefore inhibition of Amiloride-sensitive Cation Channel 2, Neuronal (ACCN2, Accession NM_{—}020039). Accordingly, utilities of VGAM217 include diagnosis, prevention and treatment of diseases and clinical conditions associated with ACCN2. BLAME (Accession NM_{—}020125) is another VGAM217 host target gene. BLAME BINDING SITE is HOST TARGET binding site found in the 3′ untranslated region of mRNA encoded by BLAME, corresponding to a HOST TARGET binding site such as BINDING SITE I, BINDING SITE II or BINDING SITE III. Table 2 illustrates the complementarity of the nucleotide sequences of BLAME BINDING SITE, designated SEQ ID:1892, to the nucleotide sequence of VGAM217 RNA, herein designated VGAM RNA, also designated SEQ ID:552.

Another function of VGAM217 is therefore inhibition of BLAME (Accession NM_{—}020125). Accordingly, utilities of VGAM217 include diagnosis, prevention and treatment of diseases and clinical conditions associated with BLAME. Distal-less Homeobox 4 (DLX4, Accession NM_{—}138281) is another VGAM217 host target gene. DLX4 BINDING SITE1 and DLX4 BINDING SITE2 are HOST TARGET binding sites found in untranslated regions of mRNA encoded by DLX4, corresponding to HOST TARGET binding sites such as BINDING SITE I, BINDING SITE II or BINDING SITE III. Table 2 illustrates the complementarity of the nucleotide sequences of DLX4 BINDING SITE1 and DLX4 BINDING SITE2, designated SEQ ID:2432 and SEQ ID:870 respectively, to the nucleotide sequence of VGAM217 RNA, herein designated VGAM RNA, also designated SEQ ID:552.

Another function of VGAM217 is therefore inhibition of Distal-less Homeobox 4 (DLX4, Accession NM_{—}138281), a gene which may regulate gene expression, morphogenesis, and differentiation. Accordingly, utilities of VGAM217 include diagnosis, prevention and treatment of diseases and clinical conditions associated with DLX4. The function of DLX4 has been established by previous studies. Using degenerate PCR, Nakamura et al. (1996) cloned a gene, which they referred to as DLX7, from human and mouse that may represent the mammalian ortholog of the newt gene NuHBox-5. They isolated a human cDNA predicting a 167-amino acid protein. The homeodomains of these genes are highly similar to those of all other vertebrate DLX genes, but there is divergence upstream of the homeodomain between the human and mouse DLX7 genes and between DLX7 and other DLX genes. They presented evidence that the mouse Dlx7 gene is alternatively spliced. By Northern blot analysis, Nakamura et al. (1996) found that DLX7 is expressed as a 2.3-kb transcript in several human cell lines. By fluorescence in situ hybridization (FISH), Nakamura et al. (1996) mapped DLX7 to 17q21.3-q22. They stated that the human DLX7 and DLX3 (OMIM Ref. No. 600525) genes are 10 kb apart and are arranged in a tail-to-tail tandem orientation, similarly to that found in mouse. Using dual-color FISH, Nakamura et al. (1996) determined that human DLX7 and HOX9B (OMIM Ref. No. 142964) lie within 2 Mb of one another. Quinn et al. (1997) undertook a DNA binding site screen of a 32-week human placental cDNA library using a consensus homeodomain binding site as a probe. They claimed that this study represented the first library screen carried out to isolate homeo box genes from the human placenta. They found that 3 homeo box genes known to be expressed in embryo, HB24 (OMIM Ref. No. 142995), GAX (OMIM Ref. No. 600535), and MSX2 (OMIM Ref. No. 123101), are also expressed in the placenta. They also identified a novel homeo box gene, designated DLX4 by them, that showed 85% sequence identity with the homeodomain encoded by the Drosophila ‘distal-less’ gene. Using FISH, they assigned DLX4 to 17q21-q22. This placed DLX4 in the same region of chromosome 17 as a member of the distal-less family gene DLX3 (OMIM Ref. No. 600525) and the HOXB homeo box gene cluster (see OMIM Ref. No. HOXB1; 142968). DLX1 (OMIM Ref. No. 600029) and DLX2 (OMIM Ref. No. 126255) are closely linked on chromosome 2; DLX5 (OMIM Ref. No. 600028) and DLX6 (OMIM Ref. No. 600030) are closely linked on chromosome 7. Thus, Quinn et al. (1997) predicted that DLX3 and DLX4 are closely linked and that they arose through gene duplication and divergence from a common ancestral precursor.

Full details of the abovementioned studies are described in the following publications, the disclosure of which are hereby incorporated by reference:
Morasso, M. I.; Yonescu, R.; Griffin, C. A.; Sargent, T. D.: Localization of human DLX8 to chromosome 17q21.3-q22 by fluorescence in situ hybridization. Mammalian Genome 8: 302-303, 1997.; and
Nakamura, S.; Stock, D. W.; Wydner, K. L.; Bollekens, J. A.; Takeshita, K.; Nagai, B. M.; Chiba, S.; Kitamura, T.; Freeland, T. M.; Zhao, Z.; Minowada, J.; Lawrence, J. B.; Weiss, K. M.

Further studies establishing the function and utilities of DLX4 are found in John Hopkins OMIM database record ID 601911, and in sited publications numbered 205 and 2056 listed in the bibliography section hereinbelow, which are also hereby incorporated by reference. Inositol 1,4,5-triphosphate Receptor, Type 2 (ITPR2, Accession NM_{—}002223) is another VGAM217 host target gene. ITPR2 BINDING SITE is HOST TARGET binding site found in the 3′ untranslated region of mRNA encoded by ITPR2, corresponding to a HOST TARGET binding site such as BINDING SITE I, BINDING SITE II or BINDING SITE III. Table 2 illustrates the complementarity of the nucleotide sequences of ITPR2 BINDING SITE, designated SEQ ID:912, to the nucleotide sequence of VGAM217 RNA, herein designated VGAM RNA, also designated SEQ ID:552.

Another function of VGAM217 is therefore inhibition of Inositol 1,4,5-triphosphate Receptor, Type 2 (ITPR2, Accession NM_{—}002223). Accordingly, utilities of VGAM217 include diagnosis, prevention and treatment of diseases and clinical conditions associated with ITPR2. Isovaleryl Coenzyme A Dehydrogenase (IVD, Accession NM_{—}002225) is another VGAM217 host target gene. IVD BINDING SITE is HOST TARGET binding site found in the 5′ untranslated region of mRNA encoded by IVD, corresponding to a HOST TARGET binding site such as BINDING SITE I, BINDING SITE II or BINDING SITE III. Table 2 illustrates the complementarity of the nucleotide sequences of IVD BINDING SITE, designated SEQ ID:913, to the nucleotide sequence of VGAM217 RNA, herein designated VGAM RNA, also designated SEQ ID:552.

Another function of VGAM217 is therefore inhibition of Isovaleryl Coenzyme A Dehydrogenase (IVD, Accession NM_{—}002225). Accordingly, utilities of VGAM217 include diagnosis, prevention and treatment of diseases and clinical conditions associated with IVD. Mitogen-activated Protein Kinase Kinase Kinase Kinase 2 (MAP4K2, Accession NM_{—}004579) is another VGAM217 host target gene. MAP4K2 BINDING SITE is HOST TARGET binding site found in the 3′ untranslated region of mRNA encoded by MAP4K2, corresponding to a HOST TARGET binding site such as BINDING SITE I, BINDING SITE II or BINDING SITE III. Table 2 illustrates the complementarity of the nucleotide sequences of MAP4K2 BINDING SITE, designated SEQ ID:1127, to the nucleotide sequence of VGAM217 RNA, herein designated VGAM RNA, also designated SEQ ID:552.

Another function of VGAM217 is therefore inhibition of Mitogen-activated Protein Kinase Kinase Kinase Kinase 2 (MAP4K2, Accession NM_{—}004579), a gene which serine/threonine protein kinase required for spore wall development. activates Jun N-terminal kinase; member of the STE20 kinase family. Accordingly, utilities of VGAM217 include diagnosis, prevention and treatment of diseases and clinical conditions associated with MAP4K2. The function of MAP4K2 has been established by previous studies. B lymphocytes that reside in the germinal center of lymphoid follicles are functionally and phenotypically distinct from those residing in the surrounding mantle zone. Various regulatory and structural genes control a complex series of differentiation and selection steps through which B cells that exit the germinal center of lymphoid follicles must pass. In differential hybridization studies to identify some of these genes, Katz et al. (1994) isolated a novel gene based on its preferential expression in tonsillar germinal center B lymphocytes. The complete nucleotide sequence predicted a 819-amino acid protein, named GC (for ‘germinal center’) kinase, with homology to serine-threonine protein kinases. Its catalytic domain was 39% and 37% identical to those of S. cerevisiae STE20 and Drosophila NinaC proteins, respectively. Northern blot analysis revealed expression of a 2.9-kb mRNA in several human tissues, including brain, lung, and placenta. In situ hybridization of tonsil tissue demonstrated preferential hybridization to the germinal center region. The expressed protein phosphorylated casein and myelin basic protein in in vitro kinase assays Ren et al. (1996) identified a mouse protein based on its interaction with the vesicular transport protein Rab8 (OMIM Ref. No. 165040). This mouse protein, termed Rab8ip, and GC kinase shared 93% amino acid sequence identity. Rab8ip/GC kinase had serine/threonine protein kinase activity manifested both as autophosphorylation and phosphorylation of casein and myelin basic protein. Based on its interaction with Rab8, the authors suggested that Rab8ip/GC kinase may modulate secretion in response to stress stimuli. Guru et al. (1997) mapped and sequenced the MEN1 (OMIM Ref. No. 131100) genomic region at 11q13. They identified MAP4K2 in the region between PYGM (OMIM Ref. No. 232600) and marker D11S4936.

Full details of the abovementioned studies are described in the following publications, the disclosure of which are hereby incorporated by reference:
Katz, P.; Whalen, G.; Kehrl, J. H.: Differential expression of a novel protein kinase in human B lymphocytes: preferential localization in the germinal center. J. Biol. Chem. 269: 16802-16809, 1994.; and
Guru, S. C.; Agarwal, S. K.; Manickam, P.; Olufemi, S.-E.; Crabtree, J. S.; Weisemann, J. M.; Kester, M. B.; Kim, Y. S.; Wang, Y.; Emmert-Buck, M. R.; Liotta, L. A.; Spiegel, A. M.; Boguski.

Further studies establishing the function and utilities of MAP4K2 are found in John Hopkins OMIM database record ID 603166, and in sited publications numbered 2661-1350 listed in the bibliography section hereinbelow, which are also hereby incorporated by reference. Polymerase (DNA directed), Gamma (POLG, Accession NM_{—}002693) is another VGAM217 host target gene. POLG BINDING SITE is HOST TARGET binding site found in the 3′ untranslated region of mRNA encoded by POLG, corresponding to a HOST TARGET binding site such as BINDING SITE I, BINDING SITE II or BINDING SITE III. Table 2 illustrates the complementarity of the nucleotide sequences of POLG BINDING SITE, designated SEQ ID:948, to the nucleotide sequence of VGAM217 RNA, herein designated VGAM RNA, also designated SEQ ID:552.

Another function of VGAM217 is therefore inhibition of Polymerase (DNA directed), Gamma (POLG, Accession NM_{—}002693). Accordingly, utilities of VGAM217 include diagnosis, prevention and treatment of diseases and clinical conditions associated with POLG. Ret Proto-oncogene (multiple endocrine neoplasia and medullary thyroid carcinoma 1, Hirschsprung disease) (RET, Accession NM_{—}020630) is another VGAM217 host target gene. RET BINDING SITE is HOST TARGET binding site found in the 3′ untranslated region of mRNA encoded by RET, corresponding to a HOST TARGET binding site such as BINDING SITE I, BINDING SITE II or BINDING SITE III. Table 2 illustrates the complementarity of the nucleotide sequences of RET BINDING SITE, designated SEQ ID:1920, to the nucleotide sequence of VGAM217 RNA, herein designated VGAM RNA, also designated SEQ ID:552.

Another function of VGAM217 is therefore inhibition of Ret Proto-oncogene (multiple endocrine neoplasia and medullary thyroid carcinoma 1, Hirschsprung disease) (RET, Accession NM_{—}020630), a gene which transduces signals for cell growth and differentiation. Accordingly, utilities of VGAM217 include diagnosis, prevention and treatment of diseases and clinical conditions associated with RET. The function of RET has been established by previous studies. Using the approach of SSCP analysis established for all 20 exons of the RET gene, Seri et al. (1997) identified 7 additional mutations among 39 sporadic and familial cases of Hirschsprung disease (detection rate 18%). They considered that the relatively low efficiency of detecting mutations of RET in Hirschsprung patients cannot be accounted for by genetic heterogeneity, which is not supported by the results of linkage analysis in pedigrees analyzed to date. Almost 74% of the point mutations in their series, as well as in other patient series, were identified among long-segment patients, who represented only 25% of the patient population. Seri et al. (1997) found a C620R substitution in a patient affected with total colonic aganglionosis; the same mutation had been found in medullary thyroid carcinoma. An R313Q mutation (164761.0026) was identified in homozygous state in a child born of consanguineous parents and was associated with the most severe Hirschsprung phenotype, namely, a total colonic aganglionosis with small bowel involvement. Eng (1996) reviewed the role of the RET protooncogene in multiple endocrine neoplasia type II and in Hirschsprung disease. Hoppener and Lips (1996) also reviewed RET gene mutations from the point of view of the molecular biology and the clinical aspects. Eng and Mulligan (1997) tabulated mutations of the RET gene in MEN2, the related sporadic tumors medullary thyroid carcinoma and pheochromocytoma, and familial and sporadic Hirschsprung disease. Germline mutations in 1 of 8 codons within RET cause the 3 subtypes of MEN2, namely, MEN2A, MEN2B, and familial medullary thyroid carcinoma. They stated that a somatic M918T mutation (164761.0013) accounts for the largest proportion of RET mutations detected in medullary thyroid carcinomas, most series showing a 30% to 50% range. It appeared that pheochromocytomas have a wider range of RET mutations. In contrast to MEN2, approximately 25% of patients with Hirschsprung disease have germline mutations scattered throughout the length of RET.

Full details of the abovementioned studies are described in the following publications, the disclosure of which are hereby incorporated by reference:
Seri, M.; Yin, L.; Barone, A.; Bolino, A.; Celli, I.; Bocciardi, R.; Pasini, B.; Ceccherini, I.; Lerone, M.; Kristoffersson, U.; Larsson, L. T.; Casasa, J. M.; Cass, D. T.; Abramowicz, M. J.; Vanderwinden, J.-M.; Kravcenkiene, I.; Baric, I.; Silengo, M.; Martucciello, G.; Romeo, G.: Frequency of RET mutations in long- and short-segment Hirschsprung disease. Hum. Mutat. 9: 243-249, 1997.; and
Hoppener, J. W. M.; Lips, C. J. M.: RET receptor tyrosine kinase gene mutations: molecular biological, physiological and clinical aspects. Europ. J. Clin. Invest. 26: 613-624, 1996.

Further studies establishing the function and utilities of RET are found in John Hopkins OMIM database record ID 164761, and in sited publications numbered 585-589, 593-592, 594-597, 2480-893, 2481-2482, 906, 861-736, 2646-741, 2935-745, 894-748, 810-816, 2483-823, 825, 829-828, 1162-1164, 293 and 1167-1173 listed in the bibliography section hereinbelow, which are also hereby incorporated by reference. Sal-like 2 (Drosophila) (SALL2, Accession XM_{—}033473) is another VGAM217 host target gene. SALL2 BINDING SITE is HOST TARGET binding site found in the 3′ untranslated region of mRNA encoded by SALL2, corresponding to a HOST TARGET binding site such as BINDING SITE I, BINDING SITE II or BINDING SITE III. Table 2 illustrates the complementarity of the nucleotide sequences of SALL2 BINDING SITE, designated SEQ ID:2672, to the nucleotide sequence of VGAM217 RNA, herein designated VGAM RNA, also designated SEQ ID:552.

Another function of VGAM217 is therefore inhibition of Sal-like 2 (Drosophila) (SALL2, Accession XM_{—}033473). Accordingly, utilities of VGAM217 include diagnosis, prevention and treatment of diseases and clinical conditions associated with SALL2. Solute Carrier Family 9 (sodium/hydrogen exchanger), Isoform 1 (antiporter, Na+/H+, amiloride sensitive) (SLC9A1, Accession XM_{—}046881) is another VGAM217 host target gene. SLC9A1 BINDING SITE is HOST TARGET binding site found in the 3′ untranslated region of mRNA encoded by SLC9A1, corresponding to a HOST TARGET binding site such as BINDING SITE I, BINDING SITE II or BINDING SITE III. Table 2 illustrates the complementarity of the nucleotide sequences of SLC9A1 BINDING SITE, designated SEQ ID:2886, to the nucleotide sequence of VGAM217 RNA, herein designated VGAM RNA, also designated SEQ ID:552.

Another function of VGAM217 is therefore inhibition of Solute Carrier Family 9 (sodium/hydrogen exchanger), Isoform 1 (antiporter, Na+/H+, amiloride sensitive) (SLC9A1, Accession XM_{—}046881), a gene which is involved in ph regulation to eliminate acids generated by active metabolism or to counter adverse environmental conditions. Accordingly, utilities of VGAM217 include diagnosis, prevention and treatment of diseases and clinical conditions associated with SLC9A1. The function of SLC9A1 has been established by previous studies. Denker et al. (2000) showed that the plasma membrane ion exchanger NHE1 acts as an anchor for actin filaments to control the integrity of the cortical cytoskeleton. This occurs through a previously unrecognized structural link between NHE1 and the actin-binding proteins ezrin (OMIM Ref. No. 123900), radixin (OMIM Ref. No. 179410), and moesin (OMIM Ref. No. 309845), which are collectively referred to as ERM proteins. NHE1 and ERM proteins were found to associate directly and colocalize in lamellipodia. Fibroblasts expressing NHE1 with mutations that disrupted binding with ERM proteins but not ion translocation had impaired organization of focal adhesions and actin stress fibers and an irregular cell shape. Denker et al. (2000) proposed a structural role for NHE1 in regulating the cortical cytoskeleton that is independent of its function as an ion exchanger. The genomic probe reported by Mattei et al. (1987) was used to map the APNH gene to 1p36.1-p35 by in situ hybridization (Mattei et al., 1988). Mattei et al. (1989) used in situ hybridization of the human cDNA probe to map the antiporter gene to the distal portion of mouse chromosome 4 and to the long arm of Chinese hamster chromosome 2, confirming the conserved homology between the distal part of human chromosome 1p, the mouse distal 4, and Chinese hamster distal 2q. By the analysis of fragment length variations in recombinant inbred strains, Morahan and Rakar (1993) likewise mapped the Nhe1 gene to mouse chromosome 4, between Lck and Akp2. Lifton et al. (1990) used genomic clones of the SLC9A1 gene to identify 2 polymorphisms. Using these RFLPs in 59 reference families, they found that the antiporter gene lies 3 cM proximal to the RH locus. Dudley et al. (1990) PCR-amplified a 376-bp fragment corresponding to the 5-prime end of SLC9A1 and detected a polymorphism within this fragment by denaturing gradient gel electrophoresis. By genetic linkage studies, they mapped SLC9A1 telomeric to D1S57 and close to RH (OMIM Ref. No. 111700) and ALPL (OMIM Ref. No. 171760). They pointed out that SLC9A1 is a plausible candidate gene for human essential hypertension.

Full details of the abovementioned studies are described in the following publications, the disclosure of which are hereby incorporated by reference:
Denker, S. P.; Huang, D. C.; Orlowski, J.; Furthmayr, H.; Barber, D. L.: Direct binding of the Na—H exchanger NHE1 to ERM proteins regulates the cortical cytoskeleton and cell shape independently of H(+) translocation. Molec. Cell 6: 1425-1436, 2000.; and
Dudley, C. R. K.; Giuffra, L. A.; Tippett, P.; Kidd, K. K.; Reeders, S. T.: The Na+/H+ antiporter: a ‘melt’ polymorphism allows regional mapping to the short arm of chromosome 1. Hum. G.

Further studies establishing the function and utilities of SLC9A1 are found in John Hopkins OMIM database record ID 107310, and in sited publications numbered 907-916 listed in the bibliography section hereinbelow, which are also hereby incorporated by reference. Zinc Finger Protein 132 (clone pHZ-12) (ZNF132, Accession NM_{—}003433) is another VGAM217 host target gene. ZNF132 BINDING SITE is HOST TARGET binding site found in the 5′ untranslated region of mRNA encoded by ZNF132, corresponding to a HOST TARGET binding site such as BINDING SITE I, BINDING SITE II or BINDING SITE III. Table 2 illustrates the complementarity of the nucleotide sequences of ZNF132 BINDING SITE, designated SEQ ID: 1020, to the nucleotide sequence of VGAM217 RNA, herein designated VGAM RNA, also designated SEQ ID:552.

Another function of VGAM217 is therefore inhibition of Zinc Finger Protein 132 (clone pHZ-12) (ZNF132, Accession NM_{—}003433). Accordingly, utilities of VGAM217 include diagnosis, prevention and treatment of diseases and clinical conditions associated with ZNF132. AD-020 (Accession NM_{—}020141) is another VGAM217 host target gene. AD-020 BINDING SITE1 and AD-020 BINDING SITE2 are HOST TARGET binding sites found in untranslated regions of mRNA encoded by AD-020, corresponding to HOST TARGET binding sites such as BINDING SITE I, BINDING SITE II or BINDING SITE III. Table 2 illustrates the complementarity of the nucleotide sequences of AD-020 BINDING SITE1 and AD-020 BINDING SITE2, designated SEQ ID: 1893 and SEQ ID:2524 respectively, to the nucleotide sequence of VGAM217 RNA, herein designated VGAM RNA, also designated SEQ ID:552.

Another function of VGAM217 is therefore inhibition of AD-020 (Accession NM_{—}020141). Accordingly, utilities of VGAM217 include diagnosis, prevention and treatment of diseases and clinical conditions associated with AD-020. DJ473B4 (Accession NM_{—}019556) is another VGAM217 host target gene. DJ473B4 BINDING SITE is HOST TARGET binding site found in the 3′ untranslated region of mRNA encoded by DJ473B4, corresponding to a HOST TARGET binding site such as BINDING SITE I, BINDING SITE II or BINDING SITE III. Table 2 illustrates the complementarity of the nucleotide sequences of DJ473B4 BINDING SITE, designated SEQ ID:1881, to the nucleotide sequence of VGAM217 RNA, herein designated VGAM RNA, also designated SEQ ID:552.

Another function of VGAM217 is therefore inhibition of DJ473B4 (Accession NM_{—}019556). Accordingly, utilities of VGAM217 include diagnosis, prevention and treatment of diseases and clinical conditions associated with DJ473B4. KIAA0475 (Accession NM_{—}014864) is another VGAM217 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID:1571, to the nucleotide sequence of VGAM217 RNA, herein designated VGAM RNA, also designated SEQ ID:552.

Another function of VGAM217 is therefore inhibition of KIAA0475 (Accession NM_{—}014864). Accordingly, utilities of VGAM217 include diagnosis, prevention and treatment of diseases and clinical conditions associated with KIAA0475. KIAA1729 (Accession XM_{—}114418) is another VGAM217 host target gene. KIAA1729 BINDING SITE is HOST TARGET binding site found in the 3′ untranslated region of mRNA encoded by KIAA1729, corresponding to a HOST TARGET binding site such as BINDING SITE I, BINDING SITE II or BINDING SITE III. Table 2 illustrates the complementarity of the nucleotide sequences of KIAA1729 BINDING SITE, designated SEQ ID:3458, to the nucleotide sequence of VGAM217 RNA, herein designated VGAM RNA, also designated SEQ ID:552.

Another function of VGAM217 is therefore inhibition of KIAA1729 (Accession XM_{—}114418). Accordingly, utilities of VGAM217 include diagnosis, prevention and treatment of diseases and clinical conditions associated with KIAA1729. Phytanoyl-CoA Hydroxylase Interacting Protein (PHYHIP, Accession NM_{—}014759) is another VGAM217 host target gene. PHYHIP BINDING SITE is HOST TARGET binding site found in the 3′ untranslated region of mRNA encoded by PHYHIP, corresponding to a HOST TARGET binding site such as BINDING SITE I, BINDING SITE II or BINDING SITE III. Table 2 illustrates the complementarity of the nucleotide sequences of PHYHIP BINDING SITE, designated SEQ ID:1541, to the nucleotide sequence of VGAM217 RNA, herein designated VGAM RNA, also designated SEQ ID:552.

Another function of VGAM217 is therefore inhibition of Phytanoyl-CoA Hydroxylase Interacting Protein (PHYHIP, Accession NM_{—}014759). Accordingly, utilities of VGAM217 include diagnosis, prevention and treatment of diseases and clinical conditions associated with PHYHIP. SCAMP-4 (Accession NM_{—}079834) is another VGAM217 host target gene. SCAMP-4 BINDING SITE is HOST TARGET binding site found in the 3′ untranslated region of mRNA encoded by SCAMP-4, corresponding to a HOST TARGET binding site such as BINDING SITE I, BINDING SITE II or BINDING SITE III. Table 2 illustrates the complementarity of the nucleotide sequences of SCAMP-4 BINDING SITE, designated SEQ ID:2372, to the nucleotide sequence of VGAM217 RNA, herein designated VGAM RNA, also designated SEQ ID:552.

Another function of VGAM217 is therefore inhibition of SCAMP-4 (Accession NM_{—}079834). Accordingly, utilities of VGAM217 include diagnosis, prevention and treatment of diseases and clinical conditions associated with SCAMP-4. ST6GalNAcl (Accession NM_{—}018414) is another VGAM217 host target gene. ST6GalNAcl BINDING SITE is HOST TARGET binding site found in the 3′ untranslated region of mRNA encoded by ST6GalNAcl, corresponding to a HOST TARGET binding site such as BINDING SITE I, BINDING SITE II or BINDING SITE III. Table 2 illustrates the complementarity of the nucleotide sequences of ST6GalNAcl BINDING SITE, designated SEQ ID:1824, to the nucleotide sequence of VGAM217 RNA, herein designated VGAM RNA, also designated SEQ ID:552.

Another function of VGAM217 is therefore inhibition of ST6GalNAcl (Accession NM_{—}018414). Accordingly, utilities of VGAM217 include diagnosis, prevention and treatment of diseases and clinical conditions associated with ST6GalNAcl. Testis-specific Transcript, Y-linked 2 (TTTY2, Accession XM_{—}099029) is another VGAM217 host target gene. TTTY2 BINDING SITE is HOST TARGET binding site found in the 5′ untranslated region of mRNA encoded by TTTY2, corresponding to a HOST TARGET binding site such as BINDING SITE I, BINDING SITE II or BINDING SITE III. Table 2 illustrates the complementarity of the nucleotide sequences of TTTY2 BINDING SITE, designated SEQ ID:3396, to the nucleotide sequence of VGAM217 RNA, herein designated VGAM RNA, also designated SEQ ID:552.

Another function of VGAM217 is therefore inhibition of Testis-specific Transcript, Y-linked 2 (TTTY2, Accession XM_{—}099029). Accordingly, utilities of VGAM217 include diagnosis, prevention and treatment of diseases and clinical conditions associated with TTTY2. LOC126917 (Accession XM_{—}059091) is another VGAM217 host target gene. LOC126917 BINDING SITE is HOST TARGET binding site found in the 3′ untranslated region of mRNA encoded by LOC126917, corresponding to a HOST TARGET binding site such as BINDING SITE I, BINDING SITE II or BINDING SITE III. Table 2 illustrates the complementarity of the nucleotide sequences of LOC126917 BINDING SITE, designated SEQ ID:3001, to the nucleotide sequence of VGAM217 RNA, herein designated VGAM RNA, also designated SEQ ID:552.

Another function of VGAM217 is therefore inhibition of LOC126917 (Accession XM_{—}059091). Accordingly, utilities of VGAM217 include diagnosis, prevention and treatment of diseases and clinical conditions associated with LOC126917. LOC128077 (Accession XM_{—}059208) is another VGAM217 host target gene. LOC128077 BINDING SITE is HOST TARGET binding site found in the 3′ untranslated region of mRNA encoded by LOC128077, corresponding to a HOST TARGET binding site such as BINDING SITE I, BINDING SITE II or BINDING SITE III. Table 2 illustrates the complementarity of the nucleotide sequences of LOC128077 BINDING SITE, designated SEQ ID:3007, to the nucleotide sequence of VGAM217 RNA, herein designated VGAM RNA, also designated SEQ ID:552.

Another function of VGAM217 is therefore inhibition of LOC128077 (Accession XM_{—}059208). Accordingly, utilities of VGAM217 include diagnosis, prevention and treatment of diseases and clinical conditions associated with LOC128077. LOC152485 (Accession XM_{—}087479) is another VGAM217 host target gene. LOC152485 BINDING SITE is HOST TARGET binding site found in the 3′ untranslated region of mRNA encoded by LOC152485, corresponding to a HOST TARGET binding site such as BINDING SITE I, BINDING SITE II or BINDING SITE III. Table 2 illustrates the complementarity of the nucleotide sequences of LOC152485 BINDING SITE, designated SEQ ID:3171, to the nucleotide sequence of VGAM217 RNA, herein designated VGAM RNA, also designated SEQ ID:552.

Another function of VGAM217 is therefore inhibition of LOC152485 (Accession XM_{—}087479). Accordingly, utilities of VGAM217 include diagnosis, prevention and treatment of diseases and clinical conditions associated with LOC152485. LOC159148 (Accession XM_{—}099030) is another VGAM217 host target gene. LOC159148 BINDING SITE is HOST TARGET binding site found in the 5′ untranslated region of mRNA encoded by LOC159148, corresponding to a HOST TARGET binding site such as BINDING SITE I, BINDING SITE II or BINDING SITE III. Table 2 illustrates the complementarity of the nucleotide sequences of LOC159148 BINDING SITE, designated SEQ ID:3397, to the nucleotide sequence of VGAM217 RNA, herein designated VGAM RNA, also designated SEQ ID:552.

Another function of VGAM217 is therefore inhibition of LOC159148 (Accession XM_{—}099030). Accordingly, utilities of VGAM217 include diagnosis, prevention and treatment of diseases and clinical conditions associated with LOC159148. LOC159989 (Accession XM_{—}089976) is another VGAM217 host target gene. LOC159989 BINDING SITE is HOST TARGET binding site found in the 3′ untranslated region of mRNA encoded by LOC159989, corresponding to a HOST TARGET binding site such as BINDING SITE I, BINDING SITE II or BINDING SITE III. Table 2 illustrates the complementarity of the nucleotide sequences of LOC159989 BINDING SITE, designated SEQ ID:3238, to the nucleotide sequence of VGAM217 RNA, herein designated VGAM RNA, also designated SEQ ID:552.

Another function of VGAM217 is therefore inhibition of LOC159989 (Accession XM_{—}089976). Accordingly, utilities of VGAM217 include diagnosis, prevention and treatment of diseases and clinical conditions associated with LOC159989. LOC164584 (Accession XM_{—}092883) is another VGAM217 host target gene. LOC164584 BINDING SITE is HOST TARGET binding site found in the 5′ untranslated region of mRNA encoded by LOC164584, corresponding to a HOST TARGET binding site such as BINDING SITE I, BINDING SITE II or BINDING SITE III. Table 2 illustrates the complementarity of the nucleotide sequences of LOC164584 BINDING SITE, designated SEQ ID:3250, to the nucleotide sequence of VGAM217 RNA, herein designated VGAM RNA, also designated SEQ ID:552.

Another function of VGAM217 is therefore inhibition of LOC164584 (Accession XM_{—}092883). Accordingly, utilities of VGAM217 include diagnosis, prevention and treatment of diseases and clinical conditions associated with LOC164584. FIG. 1 further provides a conceptual description of a novel bioinformatically detected viral gene of the present invention, referred to here as Viral Genomic Address Messenger 218 (VGAM218) viral gene, which modulates expression of respective host target genes thereof, the function and utility of which host target genes is known in the art.

VGAM218 is a novel bioinformatically detected regulatory, non protein coding, viral micro RNA (miRNA) gene. The method by which VGAM218 was detected is described hereinabove with reference to FIGS. 1-8.

VGAM218 gene, herein designated VGAM GENE, is a viral gene contained in the genome of Vaccinia Virus. VGAM218 host target gene, herein designated VGAM HOST TARGET GENE, is a human gene contained in the human genome.

VGAM218 gene encodes a VGAM218 precursor RNA, herein designated VGAM PRECURSOR RNA. Similar to other miRNA genes, and unlike most ordinary genes, VGAM218 precursor RNA does not encode a protein. A nucleotide sequence identical or highly similar to the nucleotide sequence of VGAM218 precursor RNA is designated SEQ ID:204, and is provided hereinbelow with reference to the sequence listing part. Nucleotide sequence SEQ ID:204 is located at position 114325 relative to the genome of Vaccinia Virus.

VGAM218 precursor RNA folds onto itself, forming VGAM218 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM218 folded precursor RNA into VGAM218 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM218 RNA is designated SEQ ID:553, and is provided hereinbelow with reference to the sequence listing part.

VGAM218 host target gene, herein designated VGAM HOST TARGET GENE, encodes a corresponding messenger RNA, VGAM218 host target RNA, herein designated VGAM HOST TARGET RNA. VGAM218 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM218 RNA, herein designated VGAM RNA, binds complementarily to one or more host target binding sites located in untranslated regions of VGAM218 host target RNA, herein designated VGAM HOST TARGET RNA. This complementary binding is due to the fact that the nucleotide sequence of VGAM218 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM218 RNA, herein designated VGAM RNA, may have a different number of host target binding sites in untranslated regions of a VGAM218 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM218 RNA, herein designated VGAM RNA, to host target binding sites on VGAM218 host target RNA, herein designated VGAM HOST TARGET RNA, such as BINDING SITE I, BINDING SITE II and BINDING SITE III, inhibits translation of VGAM218 host target RNA into VGAM218 host target protein, herein designated VGAM HOST TARGET PROTEIN. VGAM host target protein is therefore outlined by a broken line.

It is appreciated that VGAM218 host target gene, herein designated VGAM HOST TARGET GENE, in fact represents a plurality of VGAM218 host target genes. The mRNA of each one of this plurality of VGAM218 host target genes comprises one or more host target binding sites, each having a nucleotide sequence which is at least partly complementary to VGAM218 RNA, herein designated VGAM RNA, and which when bound by VGAM218 RNA causes inhibition of translation of respective one or more VGAM218 host target proteins.

It is further appreciated by one skilled in the art that the mode of translational inhibition illustrated by FIG. 1 with specific reference to translational inhibition exerted by VGAM218 gene, herein designated VGAM GENE, on one or more VGAM218 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM218 is inhibition of expression of host target genes, as part of a novel viral mechanism of attacking a host. Accordingly, utilities of VGAM218 include diagnosis, prevention and treatment of viral infection by Vaccinia Virus. Specific functions, and accordingly utilities, of VGAM218 correlate with, and may be deduced from, the identity of the host target genes which VGAM218 binds and inhibits, and the function of these host target genes, as elaborated hereinbelow.

Nucleotide sequences of the VGAM218 precursor RNA, herein designated VGAM PRECURSOR RNA, and of the ‘diced’ VGAM218 RNA, herein designated VGAM RNA, and a schematic representation of the secondary folding of VGAM218 folded precursor RNA, herein designated VGAM FOLDED PRECURSOR RNA, of VGAM218 are further described hereinbelow with reference to Table 1.

Nucleotide sequences of host target binding sites, such as BINDING SITE-I, BINDING SITE-II and BINDING SITE-III of FIG. 1, found on VGAM218 host target RNA, and schematic representation of the complementarity of each of these host target binding sites to VGAM218 RNA, herein designated VGAM RNA, are described hereinbelow with reference to Table 2.

As mentioned hereinabove with reference to FIG. 1, a function of VGAM218 gene, herein designated VGAM is inhibition of expression of VGAM218 target genes. It is appreciated that specific functions, and accordingly utilities, of VGAM218 correlate with, and may be deduced from, the identity of the target genes which VGAM218 binds and inhibits, and the function of these target genes, as elaborated hereinbelow.

Recombination Activating Gene 1 (RAG1, Accession NM_{—}000448) is a VGAM218 host target gene. RAG1 BINDING SITE is HOST TARGET binding site found in the 3′ untranslated region of mRNA encoded by RAG1, corresponding to a HOST TARGET binding site such as BINDING SITE I, BINDING SITE II or BINDING SITE III. Table 2 illustrates the complementarity of the nucleotide sequences of RAG1 BINDING SITE, designated SEQ ID:745, to the nucleotide sequence of VGAM218 RNA, herein designated VGAM RNA, also designated SEQ ID:553.

A function of VGAM218 is therefore inhibition of Recombination Activating Gene 1 (RAG1, Accession NM_{—}000448). Accordingly, utilities of VGAM218 include diagnosis, prevention and treatment of diseases and clinical conditions associated with RAG1. LOC221964 (Accession XM_{—}168342) is another VGAM218 host target gene. LOC221964 BINDING SITE is HOST TARGET binding site found in the 3′ untranslated region of mRNA encoded by LOC221964, corresponding to a HOST TARGET binding site such as BINDING SITE I, BINDING SITE II or BINDING SITE III. Table 2 illustrates the complementarity of the nucleotide sequences of LOC221964 BINDING SITE, designated SEQ ID:3628, to the nucleotide sequence of VGAM218 RNA, herein designated VGAM RNA, also designated SEQ ID:553.

Another function of VGAM218 is therefore inhibition of LOC221964 (Accession XM_{—}168342). Accordingly, utilities of VGAM218 include diagnosis, prevention and treatment of diseases and clinical conditions associated with LOC221964. FIG. 1 further provides a conceptual description of a novel bioinformatically detected viral gene of the present invention, referred to here as Viral Genomic Address Messenger 219 (VGAM219) viral gene, which modulates expression of respective host target genes thereof, the function and utility of which host target genes is known in the art.

VGAM219 is a novel bioinformatically detected regulatory, non protein coding, viral micro RNA (miRNA) gene. The method by which VGAM219 was detected is described hereinabove with reference to FIGS. 1-8.

VGAM219 gene, herein designated VGAM GENE, is a viral gene contained in the genome of Vaccinia Virus. VGAM219 host target gene, herein designated VGAM HOST TARGET GENE, is a human gene contained in the human genome.

VGAM219 gene encodes a VGAM219 precursor RNA, herein designated VGAM PRECURSOR RNA. Similar to other miRNA genes, and unlike most ordinary genes, VGAM219 precursor RNA does not encode a protein. A nucleotide sequence identical or highly similar to the nucleotide sequence of VGAM219 precursor RNA is designated SEQ ID:205, and is provided hereinbelow with reference to the sequence listing part. Nucleotide sequence SEQ ID:205 is located at position 115962 relative to the genome of Vaccinia Virus.

VGAM219 precursor RNA folds onto itself, forming VGAM219 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM219 folded precursor RNA into VGAM219 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM219 RNA is designated SEQ ID:554, and is provided hereinbelow with reference to the sequence listing part.

VGAM219 host target gene, herein designated VGAM HOST TARGET GENE, encodes a corresponding messenger RNA, VGAM219 host target RNA, herein designated VGAM HOST TARGET RNA. VGAM219 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM219 RNA, herein designated VGAM RNA, binds complementarily to one or more host target binding sites located in untranslated regions of VGAM219 host target RNA, herein designated VGAM HOST TARGET RNA. This complementary binding is due to the fact that the nucleotide sequence of VGAM219 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM219 RNA, herein designated VGAM RNA, may have a different number of host target binding sites in untranslated regions of a VGAM219 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM219 RNA, herein designated VGAM RNA, to host target binding sites on VGAM219 host target RNA, herein designated VGAM HOST TARGET RNA, such as BINDING SITE I, BINDING SITE II and BINDING SITE III, inhibits translation of VGAM219 host target RNA into VGAM219 host target protein, herein designated VGAM HOST TARGET PROTEIN. VGAM host target protein is therefore outlined by a broken line.

It is appreciated that VGAM219 host target gene, herein designated VGAM HOST TARGET GENE, in fact represents a plurality of VGAM219 host target genes. The mRNA of each one of this plurality of VGAM219 host target genes comprises one or more host target binding sites, each having a nucleotide sequence which is at least partly complementary to VGAM219 RNA, herein designated VGAM RNA, and which when bound by VGAM219 RNA causes inhibition of translation of respective one or more VGAM219 host target proteins.

It is further appreciated by one skilled in the art that the mode of translational inhibition illustrated by FIG. 1 with specific reference to translational inhibition exerted by VGAM219 gene, herein designated VGAM GENE, on one or more VGAM219 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM219 is inhibition of expression of host target genes, as part of a novel viral mechanism of attacking a host. Accordingly, utilities of VGAM219 include diagnosis, prevention and treatment of viral infection by Vaccinia Virus. Specific functions, and accordingly utilities, of VGAM219 correlate with, and may be deduced from, the identity of the host target genes which VGAM219 binds and inhibits, and the function of these host target genes, as elaborated hereinbelow.

Nucleotide sequences of the VGAM219 precursor RNA, herein designated VGAM PRECURSOR RNA, and of the ‘diced’ VGAM219 RNA, herein designated VGAM RNA, and a schematic representation of the secondary folding of VGAM219 folded precursor RNA, herein designated VGAM FOLDED PRECURSOR RNA, of VGAM219 are further described hereinbelow with reference to Table 1.

Nucleotide sequences of host target binding sites, such as BINDING SITE-I, BINDING SITE-II and BINDING SITE-III of FIG. 1, found on VGAM219 host target RNA, and schematic representation of the complementarity of each of these host target binding sites to VGAM219 RNA, herein designated VGAM RNA, are described hereinbelow with reference to Table 2.

As mentioned hereinabove with reference to FIG. 1, a function of VGAM219 gene, herein designated VGAM is inhibition of expression of VGAM219 target genes. It is appreciated that specific functions, and accordingly utilities, of VGAM219 correlate with, and may be deduced from, the identity of the target genes which VGAM219 binds and inhibits, and the function of these target genes, as elaborated hereinbelow.

A Disintegrin and Metalloproteinase Domain 12 (meltrin alpha) (ADAM12, Accession NM_{—}003474) is a VGAM219 host target gene. ADAM12 BINDING SITE is HOST TARGET binding site found in the 3′ untranslated region of mRNA encoded by ADAM12, corresponding to a HOST TARGET binding site such as BINDING SITE I, BINDING SITE II or BINDING SITE III. Table 2 illustrates the complementarity of the nucleotide sequences of ADAM12 BINDING SITE, designated SEQ ID: 1030, to the nucleotide sequence of VGAM219 RNA, herein designated VGAM RNA, also designated SEQ ID:554.

A function of VGAM219 is therefore inhibition of A Disintegrin and Metalloproteinase Domain 12 (meltrin alpha) (ADAM12, Accession NM_{—}003474), a gene which involved in skeletal muscle regeneration, specifically at the onset of cell fusion. Accordingly, utilities of VGAM219 include diagnosis, prevention and treatment of diseases and clinical conditions associated with ADAM12. The function of ADAM12 and its association with various diseases and clinical conditions, has been established by previous studies, as described hereinabove with reference to VGAM199. KIAA1789 (Accession XM_{—}040486) is another VGAM219 host target gene. KIAA1789 BINDING SITE is HOST TARGET binding site found in the 5′ untranslated region of mRNA encoded by KIAA1789, corresponding to a HOST TARGET binding site such as BINDING SITE I, BINDING SITE II or BINDING SITE III. Table 2 illustrates the complementarity of the nucleotide sequences of KIAA1789 BINDING SITE, designated SEQ ID:2780, to the nucleotide sequence of VGAM219 RNA, herein designated VGAM RNA, also designated SEQ ID:554.

Another function of VGAM219 is therefore inhibition of KIAA1789 (Accession XM_{—}040486). Accordingly, utilities of VGAM219 include diagnosis, prevention and treatment of diseases and clinical conditions associated with KIAA1789. FIG. 1 further provides a conceptual description of a novel bioinformatically detected viral gene of the present invention, referred to here as Viral Genomic Address Messenger 220 (VGAM220) viral gene, which modulates expression of respective host target genes thereof, the function and utility of which host target genes is known in the art.

VGAM220 is a novel bioinformatically detected regulatory, non protein coding, viral micro RNA (miRNA) gene. The method by which VGAM220 was detected is described hereinabove with reference to FIGS. 1-8.

VGAM220 gene, herein designated VGAM GENE, is a viral gene contained in the genome of Vaccinia Virus. VGAM220 host target gene, herein designated VGAM HOST TARGET GENE, is a human gene contained in the human genome.

VGAM220 gene encodes a VGAM220 precursor RNA, herein designated VGAM PRECURSOR RNA. Similar to other miRNA genes, and unlike most ordinary genes, VGAM220 precursor RNA does not encode a protein. A nucleotide sequence identical or highly similar to the nucleotide sequence of VGAM220 precursor RNA is designated SEQ ID:206, and is provided hereinbelow with reference to the sequence listing part. Nucleotide sequence SEQ ID:206 is located at position 115869 relative to the genome of Vaccinia Virus.

VGAM220 precursor RNA folds onto itself, forming VGAM220 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM220 folded precursor RNA into VGAM220 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM220 RNA is designated SEQ ID:555, and is provided hereinbelow with reference to the sequence listing part.

VGAM220 host target gene, herein designated VGAM HOST TARGET GENE, encodes a corresponding messenger RNA, VGAM220 host target RNA, herein designated VGAM HOST TARGET RNA. VGAM220 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM220 RNA, herein designated VGAM RNA, binds complementarily to one or more host target binding sites located in untranslated regions of VGAM220 host target RNA, herein designated VGAM HOST TARGET RNA. This complementary binding is due to the fact that the nucleotide sequence of VGAM220 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM220 RNA, herein designated VGAM RNA, may have a different number of host target binding sites in untranslated regions of a VGAM220 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM220 RNA, herein designated VGAM RNA, to host target binding sites on VGAM220 host target RNA, herein designated VGAM HOST TARGET RNA, such as BINDING SITE I, BINDING SITE II and BINDING SITE III, inhibits translation of VGAM220 host target RNA into VGAM220 host target protein, herein designated VGAM HOST TARGET PROTEIN. VGAM host target protein is therefore outlined by a broken line.

It is appreciated that VGAM220 host target gene, herein designated VGAM HOST TARGET GENE, in fact represents a plurality of VGAM220 host target genes. The mRNA of each one of this plurality of VGAM220 host target genes comprises one or more host target binding sites, each having a nucleotide sequence which is at least partly complementary to VGAM220 RNA, herein designated VGAM RNA, and which when bound by VGAM220 RNA causes inhibition of translation of respective one or more VGAM220 host target proteins.

It is further appreciated by one skilled in the art that the mode of translational inhibition illustrated by FIG. 1 with specific reference to translational inhibition exerted by VGAM220 gene, herein designated VGAM GENE, on one or more VGAM220 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM220 is inhibition of expression of host target genes, as part of a novel viral mechanism of attacking a host. Accordingly, utilities of VGAM220 include diagnosis, prevention and treatment of viral infection by Vaccinia Virus. Specific functions, and accordingly utilities, of VGAM220 correlate with, and may be deduced from, the identity of the host target genes which VGAM220 binds and inhibits, and the function of these host target genes, as elaborated hereinbelow.

Nucleotide sequences of the VGAM220 precursor RNA, herein designated VGAM PRECURSOR RNA, and of the ‘diced’ VGAM220 RNA, herein designated VGAM RNA, and a schematic representation of the secondary folding of VGAM220 folded precursor RNA, herein designated VGAM FOLDED PRECURSOR RNA, of VGAM220 are further described hereinbelow with reference to Table 1.

Nucleotide sequences of host target binding sites, such as BINDING SITE-I, BINDING SITE-II and BINDING SITE-III of FIG. 1, found on VGAM220 host target RNA, and schematic representation of the complementarity of each of these host target binding sites to VGAM220 RNA, herein designated VGAM RNA, are described hereinbelow with reference to Table 2.

As mentioned hereinabove with reference to FIG. 1, a function of VGAM220 gene, herein designated VGAM is inhibition of expression of VGAM220 target genes. It is appreciated that specific functions, and accordingly utilities, of VGAM220 correlate with, and may be deduced from, the identity of the target genes which VGAM220 binds and inhibits, and the function of these target genes, as elaborated hereinbelow.

Nuclear Receptor Sub family 4, Group A, Member 2 (NR4A2, Accession NM_{—}006186) is a VGAM220 host target gene. NR4A2 BINDING SITE is HOST TARGET binding site found in the 3′ untranslated region of mRNA encoded by NR4A2, corresponding to a HOST TARGET binding site such as BINDING SITE I, BINDING SITE II or BINDING SITE III. Table 2 illustrates the complementarity of the nucleotide sequences of NR4A2 BINDING SITE, designated SEQ ID:1276, to the nucleotide sequence of VGAM220 RNA, herein designated VGAM RNA, also designated SEQ ID:555.

A function of VGAM220 is therefore inhibition of Nuclear Receptor Sub family 4, Group A, Member 2 (NR4A2, Accession NM_{—}006186), a gene which may be a general coactivator of transcription. Accordingly, utilities of VGAM220 include diagnosis, prevention and treatment of diseases and clinical conditions associated with NR4A2. The function of NR4A2 and its association with various diseases and clinical conditions, has been established by previous studies, as described hereinabove with reference to VGAM148. LOC51145 (Accession NM_{—}016158) is another VGAM220 host target gene. LOC51145 BINDING SITE is HOST TARGET binding site found in the 3′ untranslated region of mRNA encoded by LOC51145, corresponding to a HOST TARGET binding site such as BINDING SITE I, BINDING SITE II or BINDING SITE III. Table 2 illustrates the complementarity of the nucleotide sequences of LOC51145 BINDING SITE, designated SEQ ID:1667, to the nucleotide sequence of VGAM220 RNA, herein designated VGAM RNA, also designated SEQ ID:555.

Another function of VGAM220 is therefore inhibition of LOC51145 (Accession NM_{—}016158). Accordingly, utilities of VGAM220 include diagnosis, prevention and treatment of diseases and clinical conditions associated with LOC51145. FIG. 1 further provides a conceptual description of a novel bioinformatically detected viral gene of the present invention, referred to here as Viral Genomic Address Messenger 221 (VGAM221) viral gene, which modulates expression of respective host target genes thereof, the function and utility of which host target genes is known in the art.

VGAM221 is a novel bioinformatically detected regulatory, non protein coding, viral micro RNA (miRNA) gene. The method by which VGAM221 was detected is described hereinabove with reference to FIGS. 1-8.

VGAM221 gene, herein designated VGAM GENE, is a viral gene contained in the genome of Vaccinia Virus. VGAM221 host target gene, herein designated VGAM HOST TARGET GENE, is a human gene contained in the human genome.

VGAM221 gene encodes a VGAM221 precursor RNA, herein designated VGAM PRECURSOR RNA. Similar to other miRNA genes, and unlike most ordinary genes, VGAM221 precursor RNA does not encode a protein. A nucleotide sequence identical or highly similar to the nucleotide sequence of VGAM221 precursor RNA is designated SEQ ID:207, and is provided hereinbelow with reference to the sequence listing part. Nucleotide sequence SEQ ID:207 is located at position 116795 relative to the genome of Vaccinia Virus.

VGAM221 precursor RNA folds onto itself, forming VGAM221 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM221 folded precursor RNA into VGAM221 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM221 RNA is designated SEQ ID:556, and is provided hereinbelow with reference to the sequence listing part.

VGAM221 host target gene, herein designated VGAM HOST TARGET GENE, encodes a corresponding messenger RNA, VGAM221 host target RNA, herein designated VGAM HOST TARGET RNA. VGAM221 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM221 RNA, herein designated VGAM RNA, binds complementarily to one or more host target binding sites located in untranslated regions of VGAM221 host target RNA, herein designated VGAM HOST TARGET RNA. This complementary binding is due to the fact that the nucleotide sequence of VGAM221 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM221 RNA, herein designated VGAM RNA, may have a different number of host target binding sites in untranslated regions of a VGAM221 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM221 RNA, herein designated VGAM RNA, to host target binding sites on VGAM221 host target RNA, herein designated VGAM HOST TARGET RNA, such as BINDING SITE I, BINDING SITE II and BINDING SITE III, inhibits translation of VGAM221 host target RNA into VGAM221 host target protein, herein designated VGAM HOST TARGET PROTEIN. VGAM host target protein is therefore outlined by a broken line.

It is appreciated that VGAM221 host target gene, herein designated VGAM HOST TARGET GENE, in fact represents a plurality of VGAM221 host target genes. The mRNA of each one of this plurality of VGAM221 host target genes comprises one or more host target binding sites, each having a nucleotide sequence which is at least partly complementary to VGAM221 RNA, herein designated VGAM RNA, and which when bound by VGAM221 RNA causes inhibition of translation of respective one or more VGAM221 host target proteins.

It is further appreciated by one skilled in the art that the mode of translational inhibition illustrated by FIG. 1 with specific reference to translational inhibition exerted by VGAM221 gene, herein designated VGAM GENE, on one or more VGAM221 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM221 is inhibition of expression of host target genes, as part of a novel viral mechanism of attacking a host. Accordingly, utilities of VGAM221 include diagnosis, prevention and treatment of viral infection by Vaccinia Virus. Specific functions, and accordingly utilities, of VGAM221 correlate with, and may be deduced from, the identity of the host target genes which VGAM221 binds and inhibits, and the function of these host target genes, as elaborated hereinbelow.

Nucleotide sequences of the VGAM221 precursor RNA, herein designated VGAM PRECURSOR RNA, and of the ‘diced’ VGAM221 RNA, herein designated VGAM RNA, and a schematic representation of the secondary folding of VGAM221 folded precursor RNA, herein designated VGAM FOLDED PRECURSOR RNA, of VGAM221 are further described hereinbelow with reference to Table 1.

Nucleotide sequences of host target binding sites, such as BINDING SITE-I, BINDING SITE-II and BINDING SITE-III of FIG. 1, found on VGAM221 host target RNA, and schematic representation of the complementarity of each of these host target binding sites to VGAM221 RNA, herein designated VGAM RNA, are described hereinbelow with reference to Table 2.

As mentioned hereinabove with reference to FIG. 1, a function of VGAM221 gene, herein designated VGAM is inhibition of expression of VGAM221 target genes. It is appreciated that specific functions, and accordingly utilities, of VGAM221 correlate with, and may be deduced from, the identity of the target genes which VGAM221 binds and inhibits, and the function of these target genes, as elaborated hereinbelow.

Hemochromatosis (HFE, Accession NM_{—}000410) is a VGAM221 host target gene. HFE BINDING SITE1 and HFE BINDING SITE2 are HOST TARGET binding sites found in untranslated regions of mRNA encoded by HFE, corresponding to HOST TARGET binding sites such as BINDING SITE I, BINDING SITE II or BINDING SITE III. Table 2 illustrates the complementarity of the nucleotide sequences of HFE BINDING SITE1 and HFE BINDING SITE2, designated SEQ ID:738 and SEQ ID:2464 respectively, to the nucleotide sequence of VGAM221 RNA, herein designated VGAM RNA, also designated SEQ ID:556.

A function of VGAM221 is therefore inhibition of Hemochromatosis (HFE, Accession NM_{—}000410). Accordingly, utilities of VGAM221 include diagnosis, prevention and treatment of diseases and clinical conditions associated with HFE. Pleiomorphic Adenoma Gene 1 (PLAG1, Accession NM_{—}002655) is another VGAM221 host target gene. PLAG1 BINDING SITE is HOST TARGET binding site found in the 3′ untranslated region of mRNA encoded by PLAG1, corresponding to a HOST TARGET binding site such as BINDING SITE I, BINDING SITE II or BINDING SITE III. Table 2 illustrates the complementarity of the nucleotide sequences of PLAG1 BINDING SITE, designated SEQ ID:943, to the nucleotide sequence of VGAM221 RNA, herein designated VGAM RNA, also designated SEQ ID:556.

Another function of VGAM221 is therefore inhibition of Pleiomorphic Adenoma Gene 1 (PLAG1, Accession NM_{—}002655), a gene which contains a zinc finger domain. Accordingly, utilities of VGAM221 include diagnosis, prevention and treatment of diseases and clinical conditions associated with PLAG1. The function of PLAG1 and its association with various diseases and clinical conditions, has been established by previous studies, as described hereinabove with reference to VGAM50. FLJ20371 (Accession NM_{—}017791) is another VGAM221 host target gene. FLJ20371 BINDING SITE is HOST TARGET binding site found in the 3′ untranslated region of mRNA encoded by FLJ20371, corresponding to a HOST TARGET binding site such as BINDING SITE I, BINDING SITE II or BINDING SITE III. Table 2 illustrates the complementarity of the nucleotide sequences of FLJ20371 BINDING SITE, designated SEQ ID:1752, to the nucleotide sequence of VGAM221 RNA, herein designated VGAM RNA, also designated SEQ ID:556.

Another function of VGAM221 is therefore inhibition of FLJ20371 (Accession NM_{—}017791). Accordingly, utilities of VGAM221 include diagnosis, prevention and treatment of diseases and clinical conditions associated with FLJ20371. LOC202052 (Accession XM_{—}117355) is another VGAM221 host target gene. LOC202052 BINDING SITE is HOST TARGET binding site found in the 3′ untranslated region of mRNA encoded by LOC202052, corresponding to a HOST TARGET binding site such as BINDING SITE I, BINDING SITE II or BINDING SITE III. Table 2 illustrates the complementarity of the nucleotide sequences of LOC202052 BINDING SITE, designated SEQ ID:3485, to the nucleotide sequence of VGAM221 RNA, herein designated VGAM RNA, also designated SEQ ID:556.

Another function of VGAM221 is therefore inhibition of LOC202052 (Accession XM_{—}117355). Accordingly, utilities of VGAM221 include diagnosis, prevention and treatment of diseases and clinical conditions associated with LOC202052. FIG. 1 further provides a conceptual description of a novel bioinformatically detected viral gene of the present invention, referred to here as Viral Genomic Address Messenger 222 (VGAM222) viral gene, which modulates expression of respective host target genes thereof, the function and utility of which host target genes is known in the art.

VGAM222 is a novel bioinformatically detected regulatory, non protein coding, viral micro RNA (miRNA) gene. The method by which VGAM222 was detected is described hereinabove with reference to FIGS. 1-8.

VGAM222 gene, herein designated VGAM GENE, is a viral gene contained in the genome of Vaccinia Virus. VGAM222 host target gene, herein designated VGAM HOST TARGET GENE, is a human gene contained in the human genome.

VGAM222 gene encodes a VGAM222 precursor RNA, herein designated VGAM PRECURSOR RNA. Similar to other miRNA genes, and unlike most ordinary genes, VGAM222 precursor RNA does not encode a protein. A nucleotide sequence identical or highly similar to the nucleotide sequence of VGAM222 precursor RNA is designated SEQ ID:208, and is provided hereinbelow with reference to the sequence listing part. Nucleotide sequence SEQ ID:208 is located at position 117498 relative to the genome of Vaccinia Virus.

VGAM222 precursor RNA folds onto itself, forming VGAM222 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM222 folded precursor RNA into VGAM222 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM222 RNA is designated SEQ ID:557, and is provided hereinbelow with reference to the sequence listing part.

VGAM222 host target gene, herein designated VGAM HOST TARGET GENE, encodes a corresponding messenger RNA, VGAM222 host target RNA, herein designated VGAM HOST TARGET RNA. VGAM222 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM222 RNA, herein designated VGAM RNA, binds complementarily to one or more host target binding sites located in untranslated regions of VGAM222 host target RNA, herein designated VGAM HOST TARGET RNA. This complementary binding is due to the fact that the nucleotide sequence of VGAM222 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM222 RNA, herein designated VGAM RNA, may have a different number of host target binding sites in untranslated regions of a VGAM222 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM222 RNA, herein designated VGAM RNA, to host target binding sites on VGAM222 host target RNA, herein designated VGAM HOST TARGET RNA, such as BINDING SITE I, BINDING SITE II and BINDING SITE III, inhibits translation of VGAM222 host target RNA into VGAM222 host target protein, herein designated VGAM HOST TARGET PROTEIN. VGAM host target protein is therefore outlined by a broken line.

It is appreciated that VGAM222 host target gene, herein designated VGAM HOST TARGET GENE, in fact represents a plurality of VGAM222 host target genes. The mRNA of each one of this plurality of VGAM222 host target genes comprises one or more host target binding sites, each having a nucleotide sequence which is at least partly complementary to VGAM222 RNA, herein designated VGAM RNA, and which when bound by VGAM222 RNA causes inhibition of translation of respective one or more VGAM222 host target proteins.

It is further appreciated by one skilled in the art that the mode of translational inhibition illustrated by FIG. 1 with specific reference to translational inhibition exerted by VGAM222 gene, herein designated VGAM GENE, on one or more VGAM222 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM222 is inhibition of expression of host target genes, as part of a novel viral mechanism of attacking a host. Accordingly, utilities of VGAM222 include diagnosis, prevention and treatment of viral infection by Vaccinia Virus. Specific functions, and accordingly utilities, of VGAM222 correlate with, and may be deduced from, the identity of the host target genes which VGAM222 binds and inhibits, and the function of these host target genes, as elaborated hereinbelow.

Nucleotide sequences of the VGAM222 precursor RNA, herein designated VGAM PRECURSOR RNA, and of the ‘diced’ VGAM222 RNA, herein designated VGAM RNA, and a schematic representation of the secondary folding of VGAM222 folded precursor RNA, herein designated VGAM FOLDED PRECURSOR RNA, of VGAM222 are further described hereinbelow with reference to Table 1.

Nucleotide sequences of host target binding sites, such as BINDING SITE-I, BINDING SITE-II and BINDING SITE-III of FIG. 1, found on VGAM222 host target RNA, and schematic representation of the complementarity of each of these host target binding sites to VGAM222 RNA, herein designated VGAM RNA, are described hereinbelow with reference to Table 2.

As mentioned hereinabove with reference to FIG. 1, a function of VGAM222 gene, herein designated VGAM is inhibition of expression of VGAM222 target genes. It is appreciated that specific functions, and accordingly utilities, of VGAM222 correlate with, and may be deduced from, the identity of the target genes which VGAM222 binds and inhibits, and the function of these target genes, as elaborated hereinbelow.

Eukaryotic Translation Initiation Factor 2C, 1 (EIF2C1, Accession NM_{—}012199) is a VGAM222 host target gene. EIF2C1 BINDING SITE is HOST TARGET binding site found in the 3′ untranslated region of mRNA encoded by EIF2C1, corresponding to a HOST TARGET binding site such as BINDING SITE I, BINDING SITE II or BINDING SITE III. Table 2 illustrates the complementarity of the nucleotide sequences of EIF2C1 BINDING SITE, designated SEQ ID: 1413, to the nucleotide sequence of VGAM222 RNA, herein designated VGAM RNA, also designated SEQ ID:557.

A function of VGAM222 is therefore inhibition of Eukaryotic Translation Initiation Factor 2C, 1 (EIF2C1, Accession NM_{—}012199), a gene which plays an important role in the eukaryotic peptide chain initiation process. Accordingly, utilities of VGAM222 include diagnosis, prevention and treatment of diseases and clinical conditions associated with EIF2C1. The function of EIF2C1 and its association with various diseases and clinical conditions, has been established by previous studies, as described hereinabove with reference to VGAM131. Fragile X Mental Retardation 2 (FMR2, Accession NM_{—}002025) is another VGAM222 host target gene. FMR2 BINDING SITE is HOST TARGET binding site found in the 3′ untranslated region of mRNA encoded by FMR2, corresponding to a HOST TARGET binding site such as BINDING SITE I, BINDING SITE II or BINDING SITE III. Table 2 illustrates the complementarity of the nucleotide sequences of FMR2 BINDING SITE, designated SEQ ID:888, to the nucleotide sequence of VGAM222 RNA, herein designated VGAM RNA, also designated SEQ ID:557.

Another function of VGAM222 is therefore inhibition of Fragile X Mental Retardation 2 (FMR2, Accession NM_{—}002025). Accordingly, utilities of VGAM222 include diagnosis, prevention and treatment of diseases and clinical conditions associated with FMR2. FLJ13612 (Accession NM_{—}025202) is another VGAM222 host target gene. FLJ13612 BINDING SITE is HOST TARGET binding site found in the 3′ untranslated region of mRNA encoded by FLJ13612, corresponding to a HOST TARGET binding site such as BINDING SITE I, BINDING SITE II or BINDING SITE III. Table 2 illustrates the complementarity of the nucleotide sequences of FLJ13612 BINDING SITE, designated SEQ ID:2146, to the nucleotide sequence of VGAM222 RNA, herein designated VGAM RNA, also designated SEQ ID:557.

Another function of VGAM222 is therefore inhibition of FLJ13612 (Accession NM_{—}025202). Accordingly, utilities of VGAM222 include diagnosis, prevention and treatment of diseases and clinical conditions associated with FLJ13612. FIG. 1 further provides a conceptual description of a novel bioinformatically detected viral gene of the present invention, referred to here as Viral Genomic Address Messenger 223 (VGAM223) viral gene, which modulates expression of respective host target genes thereof, the function and utility of which host target genes is known in the art.

VGAM223 is a novel bioinformatically detected regulatory, non protein coding, viral micro RNA (miRNA) gene. The method by which VGAM223 was detected is described hereinabove with reference to FIGS. 1-8.

VGAM223 gene, herein designated VGAM GENE, is a viral gene contained in the genome of Vaccinia Virus. VGAM223 host target gene, herein designated VGAM HOST TARGET GENE, is a human gene contained in the human genome.

VGAM223 gene encodes a VGAM223 precursor RNA, herein designated VGAM PRECURSOR RNA. Similar to other miRNA genes, and unlike most ordinary genes, VGAM223 precursor RNA does not encode a protein. A nucleotide sequence identical or highly similar to the nucleotide sequence of VGAM223 precursor RNA is designated SEQ ID:209, and is provided hereinbelow with reference to the sequence listing part. Nucleotide sequence SEQ ID:209 is located at position 117715 relative to the genome of Vaccinia Virus.

VGAM223 precursor RNA folds onto itself, forming VGAM223 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM223 folded precursor RNA into VGAM223 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM223 RNA is designated SEQ ID:558, and is provided hereinbelow with reference to the sequence listing part.

VGAM223 host target gene, herein designated VGAM HOST TARGET GENE, encodes a corresponding messenger RNA, VGAM223 host target RNA, herein designated VGAM HOST TARGET RNA. VGAM223 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM223 RNA, herein designated VGAM RNA, binds complementarily to one or more host target binding sites located in untranslated regions of VGAM223 host target RNA, herein designated VGAM HOST TARGET RNA. This complementary binding is due to the fact that the nucleotide sequence of VGAM223 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM223 RNA, herein designated VGAM RNA, may have a different number of host target binding sites in untranslated regions of a VGAM223 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM223 RNA, herein designated VGAM RNA, to host target binding sites on VGAM223 host target RNA, herein designated VGAM HOST TARGET RNA, such as BINDING SITE I, BINDING SITE II and BINDING SITE III, inhibits translation of VGAM223 host target RNA into VGAM223 host target protein, herein designated VGAM HOST TARGET PROTEIN. VGAM host target protein is therefore outlined by a broken line.

It is appreciated that VGAM223 host target gene, herein designated VGAM HOST TARGET GENE, in fact represents a plurality of VGAM223 host target genes. The mRNA of each one of this plurality of VGAM223 host target genes comprises one or more host target binding sites, each having a nucleotide sequence which is at least partly complementary to VGAM223 RNA, herein designated VGAM RNA, and which when bound by VGAM223 RNA causes inhibition of translation of respective one or more VGAM223 host target proteins.

It is further appreciated by one skilled in the art that the mode of translational inhibition illustrated by FIG. 1 with specific reference to translational inhibition exerted by VGAM223 gene, herein designated VGAM GENE, on one or more VGAM223 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM223 is inhibition of expression of host target genes, as part of a novel viral mechanism of attacking a host. Accordingly, utilities of VGAM223 include diagnosis, prevention and treatment of viral infection by Vaccinia Virus. Specific functions, and accordingly utilities, of VGAM223 correlate with, and may be deduced from, the identity of the host target genes which VGAM223 binds and inhibits, and the function of these host target genes, as elaborated hereinbelow.

Nucleotide sequences of the VGAM223 precursor RNA, herein designated VGAM PRECURSOR RNA, and of the ‘diced’ VGAM223 RNA, herein designated VGAM RNA, and a schematic representation of the secondary folding of VGAM223 folded precursor RNA, herein designated VGAM FOLDED PRECURSOR RNA, of VGAM223 are further described hereinbelow with reference to Table 1.

Nucleotide sequences of host target binding sites, such as BINDING SITE-I, BINDING SITE-II and BINDING SITE-III of FIG. 1, found on VGAM223 host target RNA, and schematic representation of the complementarity of each of these host target binding sites to VGAM223 RNA, herein designated VGAM RNA, are described hereinbelow with reference to Table 2.

As mentioned hereinabove with reference to FIG. 1, a function of VGAM223 gene, herein designated VGAM is inhibition of expression of VGAM223 target genes. It is appreciated that specific functions, and accordingly utilities, of VGAM223 correlate with, and may be deduced from, the identity of the target genes which VGAM223 binds and inhibits, and the function of these target genes, as elaborated hereinbelow.

Hermansky-Pudlak Syndrome 4 (HPS4, Accession NM_{—}022081) is a VGAM223 host target gene. HPS4 BINDING SITE is HOST TARGET binding site found in the 5′ untranslated region of mRNA encoded by HPS4, corresponding to a HOST TARGET binding site such as BINDING SITE I, BINDING SITE II or BINDING SITE III. Table 2 illustrates the complementarity of the nucleotide sequences of HPS4 BINDING SITE, designated SEQ ID:1977, to the nucleotide sequence of VGAM223 RNA, herein designated VGAM RNA, also designated SEQ ID:558.

A function of VGAM223 is therefore inhibition of Hermansky-Pudlak Syndrome 4 (HPS4, Accession NM_{—}022081). Accordingly, utilities of VGAM223 include diagnosis, prevention and treatment of diseases and clinical conditions associated with HPS4. Tektin 1 (TEKT1, Accession NM_{—}053285) is another VGAM223 host target gene. TEKT1 BINDING SITE is HOST TARGET binding site found in the 5′ untranslated region of mRNA encoded by TEKT1, corresponding to a HOST TARGET binding site such as BINDING SITE I, BINDING SITE II or BINDING SITE III. Table 2 illustrates the complementarity of the nucleotide sequences of TEKT1 BINDING SITE, designated SEQ ID:2359, to the nucleotide sequence of VGAM223 RNA, herein designated VGAM RNA, also designated SEQ ID:558.

Another function of VGAM223 is therefore inhibition of Tektin 1 (TEKT1, Accession NM_{—}053285). Accordingly, utilities of VGAM223 include diagnosis, prevention and treatment of diseases and clinical conditions associated with TEKT1. LOC145783 (Accession XM_{—}085231) is another VGAM223 host target gene. LOC145783 BINDING SITE is HOST TARGET binding site found in the 3′ untranslated region of mRNA encoded by LOC145783, corresponding to a HOST TARGET binding site such as BINDING SITE I, BINDING SITE II or BINDING SITE III. Table 2 illustrates the complementarity of the nucleotide sequences of LOC145783 BINDING SITE, designated SEQ ID:3082, to the nucleotide sequence of VGAM223 RNA, herein designated VGAM RNA, also designated SEQ ID:558.

Another function of VGAM223 is therefore inhibition of LOC145783 (Accession XM_{—}085231). Accordingly, utilities of VGAM223 include diagnosis, prevention and treatment of diseases and clinical conditions associated with LOC145783. LOC154321 (Accession XM_{—}087906) is another VGAM223 host target gene. LOC154321 BINDING SITE is HOST TARGET binding site found in the 3′ untranslated region of mRNA encoded by LOC154321, corresponding to a HOST TARGET binding site such as BINDING SITE I, BINDING SITE II or BINDING SITE III. Table 2 illustrates the complementarity of the nucleotide sequences of LOC154321 BINDING SITE, designated SEQ ID:3190, to the nucleotide sequence of VGAM223 RNA, herein designated VGAM RNA, also designated SEQ ID:558.

Another function of VGAM223 is therefore inhibition of LOC154321 (Accession XM_{—}087906). Accordingly, utilities of VGAM223 include diagnosis, prevention and treatment of diseases and clinical conditions associated with LOC154321. FIG. 1 further provides a conceptual description of a novel bioinformatically detected viral gene of the present invention, referred to here as Viral Genomic Address Messenger 224 (VGAM224) viral gene, which modulates expression of respective host target genes thereof, the function and utility of which host target genes is known in the art.

VGAM224 is a novel bioinformatically detected regulatory, non protein coding, viral micro RNA (miRNA) gene. The method by which VGAM224 was detected is described hereinabove with reference to FIGS. 1-8.

VGAM224 gene, herein designated VGAM GENE, is a viral gene contained in the genome of Vaccinia Virus. VGAM224 host target gene, herein designated VGAM HOST TARGET GENE, is a human gene contained in the human genome.

VGAM224 gene encodes a VGAM224 precursor RNA, herein designated VGAM PRECURSOR RNA. Similar to other miRNA genes, and unlike most ordinary genes, VGAM224 precursor RNA does not encode a protein. A nucleotide sequence identical or highly similar to the nucleotide sequence of VGAM224 precursor RNA is designated SEQ ID:210, and is provided hereinbelow with reference to the sequence listing part. Nucleotide sequence SEQ ID:210 is located at position 119904 relative to the genome of Vaccinia Virus.

VGAM224 precursor RNA folds onto itself, forming VGAM224 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM224 folded precursor RNA into VGAM224 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM224 RNA is designated SEQ ID:559, and is provided hereinbelow with reference to the sequence listing part.

VGAM224 host target gene, herein designated VGAM HOST TARGET GENE, encodes a corresponding messenger RNA, VGAM224 host target RNA, herein designated VGAM HOST TARGET RNA. VGAM224 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM224 RNA, herein designated VGAM RNA, binds complementarily to one or more host target binding sites located in untranslated regions of VGAM224 host target RNA, herein designated VGAM HOST TARGET RNA. This complementary binding is due to the fact that the nucleotide sequence of VGAM224 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM224 RNA, herein designated VGAM RNA, may have a different number of host target binding sites in untranslated regions of a VGAM224 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM224 RNA, herein designated VGAM RNA, to host target binding sites on VGAM224 host target RNA, herein designated VGAM HOST TARGET RNA, such as BINDING SITE I, BINDING SITE II and BINDING SITE III, inhibits translation of VGAM224 host target RNA into VGAM224 host target protein, herein designated VGAM HOST TARGET PROTEIN. VGAM host target protein is therefore outlined by a broken line.

It is appreciated that VGAM224 host target gene, herein designated VGAM HOST TARGET GENE, in fact represents a plurality of VGAM224 host target genes. The mRNA of each one of this plurality of VGAM224 host target genes comprises one or more host target binding sites, each having a nucleotide sequence which is at least partly complementary to VGAM224 RNA, herein designated VGAM RNA, and which when bound by VGAM224 RNA causes inhibition of translation of respective one or more VGAM224 host target proteins.

It is further appreciated by one skilled in the art that the mode of translational inhibition illustrated by FIG. 1 with specific reference to translational inhibition exerted by VGAM224 gene, herein designated VGAM GENE, on one or more VGAM224 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM224 is inhibition of expression of host target genes, as part of a novel viral mechanism of attacking a host. Accordingly, utilities of VGAM224 include diagnosis, prevention and treatment of viral infection by Vaccinia Virus. Specific functions, and accordingly utilities, of VGAM224 correlate with, and may be deduced from, the identity of the host target genes which VGAM224 binds and inhibits, and the function of these host target genes, as elaborated hereinbelow.

Nucleotide sequences of the VGAM224 precursor RNA, herein designated VGAM PRECURSOR RNA, and of the ‘diced’ VGAM224 RNA, herein designated VGAM RNA, and a schematic representation of the secondary folding of VGAM224 folded precursor RNA, herein designated VGAM FOLDED PRECURSOR RNA, of VGAM224 are further described hereinbelow with reference to Table 1.

Nucleotide sequences of host target binding sites, such as BINDING SITE-I, BINDING SITE-II and BINDING SITE-III of FIG. 1, found on VGAM224 host target RNA, and schematic representation of the complementarity of each of these host target binding sites to VGAM224 RNA, herein designated VGAM RNA, are described hereinbelow with reference to Table 2.

As mentioned hereinabove with reference to FIG. 1, a function of VGAM224 gene, herein designated VGAM is inhibition of expression of VGAM224 target genes. It is appreciated that specific functions, and accordingly utilities, of VGAM224 correlate with, and may be deduced from, the identity of the target genes which VGAM224 binds and inhibits, and the function of these target genes, as elaborated hereinbelow.

Fasciculation and Elongation Protein Zeta 1 (zygin I) (FEZ1, Accession NM_{—}022549) is a VGAM224 host target gene. FEZ1 BINDING SITE is HOST TARGET binding site found in the 3′ untranslated region of mRNA encoded by FEZ1, corresponding to a HOST TARGET binding site such as BINDING SITE I, BINDING SITE II or BINDING SITE III. Table 2 illustrates the complementarity of the nucleotide sequences of FEZ1 BINDING SITE, designated SEQ ID:1993, to the nucleotide sequence of VGAM224 RNA, herein designated VGAM RNA, also designated SEQ ID:559.

A function of VGAM224 is therefore inhibition of Fasciculation and Elongation Protein Zeta 1 (zygin I) (FEZ1, Accession NM_{—}022549), a gene which Zygin 1; may have a role in axonal outgrowth; has similarity to C. elegans UNC-76. Accordingly, utilities of VGAM224 include diagnosis, prevention and treatment of diseases and clinical conditions associated with FEZ1. The function of FEZ1 has been established by previous studies. Ishii et al. (1999) positionally cloned and characterized the FEZ1/LZTS1 (leucine zipper, putative tumor suppressor-1) gene at 8p22, a region that is lost in many tumors, including prostate, breast, head and neck, esophageal, and urinary bladder carcinomas. The predicted FEZ1 protein contained a leucine-zipper region with similarity to the DNA-binding domain of the cAMP-responsive activating transcription factor-5 (OMIM Ref. No. 606398). Northern blot analysis revealed that FEZ2 is expressed almost ubiquitously in normal tissues, although expression is most abundant in testes. FEZ1 expression was undetectable in more than 60% of epithelial tumors, but FEZ1 mutations were found in primary esophageal cancers and in a prostate cancer cell line. Transcript analysis from several FEZ1-expressing tumors revealed truncated mRNAs, including a frameshift. Alteration and inactivation of the FEZ1 gene may play a role in various human tumors. Ishii et al. (2001) showed that introduction of FEZ1/LZTS1 into FEZ1/LZTS1-negative cancer cells resulted in suppression of tumorigenicity and reduced cell growth with accumulation of cells at late S-G2/M stage of the cell cycle. Their data showed that FEZ1/LZTS1 inhibits cancer cell growth through regulation of mitosis, and that its alterations result in abnormal cell growth. Ishii et al. (1999) analyzed the nucleotide sequence of the FEZ1 gene open reading frame in 194 cancers, including 72 primary esophageal cancers. They found a point mutation in 2 primary esophageal cancers and in a prostate cancer cell line.

Full details of the abovementioned studies are described in the following publications, the disclosure of which are hereby incorporated by reference:
Ishii, H.; Baffa, R.; Numata, S.-I.; Murakumo, Y.; Rattan, S.; Inoue, H.; Mori, M.; Fidanza, V.; Alder, H.; Croce, C. M.: The FEZ1 gene at chromosome 8p22 encodes a leucine-zipper protein, and its expression is altered in multiple human tumors. Proc. Nat. Acad. Sci. 96: 3928-3933, 1999.; and
Ishii, H.; Vecchione, A.; Murakumo, Y.; Baldassarre, G.; Numata, S.; Trapasso, F.; Alder, H.; Baffa, R.; Croce, C. M.: FEZ1/LZTS1 gene at 8p22 suppresses cancer cell growth and regula.

Further studies establishing the function and utilities of FEZ1 are found in John Hopkins OMIM database record ID 606551, and in sited publications numbered 1050 listed in the bibliography section hereinbelow, which are also hereby incorporated by reference. Golgi Autoantigen, Golgin Sub family A, 4 (GOLGA4, Accession XM_{—}011069) is another VGAM224 host target gene. GOLGA4 BINDING SITE is HOST TARGET binding site found in the 5′ untranslated region of mRNA encoded by GOLGA4, corresponding to a HOST TARGET binding site such as BINDING SITE I, BINDING SITE II or BINDING SITE III. Table 2 illustrates the complementarity of the nucleotide sequences of GOLGA4 BINDING SITE, designated SEQ ID:2554, to the nucleotide sequence of VGAM224 RNA, herein designated VGAM RNA, also designated SEQ ID:559.

Another function of VGAM224 is therefore inhibition of Golgi Autoantigen, Golgin Sub family A, 4 (GOLGA4, Accession XM_{—}011069), a gene which may play a role in vesicular transport from the trans-golgi. Accordingly, utilities of VGAM224 include diagnosis, prevention and treatment of diseases and clinical conditions associated with GOLGA4. The function of GOLGA4 and its association with various diseases and clinical conditions, has been established by previous studies, as described hereinabove with reference to VGAM181. Hyaluronan Synthase 3 (HAS3, Accession NM_{—}005329) is another VGAM224 host target gene. HAS3 BINDING SITE is HOST TARGET binding site found in the 3′ untranslated region of mRNA encoded by HAS3, corresponding to a HOST TARGET binding site such as BINDING SITE I, BINDING SITE II or BINDING SITE III. Table 2 illustrates the complementarity of the nucleotide sequences of HAS3 BINDING SITE, designated SEQ ID:1199, to the nucleotide sequence of VGAM224 RNA, herein designated VGAM RNA, also designated SEQ ID:559.

Another function of VGAM224 is therefore inhibition of Hyaluronan Synthase 3 (HAS3, Accession NM_{—}005329), a gene which plays a role in hyaluronan/hyaluronic acid (ha) synthesis. Accordingly, utilities of VGAM224 include diagnosis, prevention and treatment of diseases and clinical conditions associated with HAS3. The function of HAS3 has been established by previous studies. Hyaluronan (HA) is an unbranched glycosaminoglycan composed of repeating disaccharide units. It is a major constituent of the extracellular matrix and has been implicated in development, tumorigenesis, and several diseases. HA is synthesized at the inner face of the plasma membrane and is subsequently extruded to the outside of the cell. By degenerate PCR, Spicer et al. (1997) isolated a genomic fragment of human HA synthase-3 (HAS3) and genomic and cDNA clones of mouse Has3. The amino acid sequences encoded by the partial HAS3 fragment and the corresponding region of Has3 are 99% conserved. The authors noted that the high degree of sequence conservation between specific human and mouse HASs contrasts with the lower level of identity between HASs within a species, suggesting an evolutionary conservation of functionally important residues and differences in the mode of action of the various HASs. The predicted 554-amino acid Has3 has several consensus HA-binding motifs and multiple transmembrane domains, with 2 at the N terminus and a cluster at the C terminus. Expression of Has3 in COS-1 cells led to high levels of HA biosynthesis. Northern blot analysis of the mouse embryo showed that Has3 is predominantly expressed at late gestation as a major, approximately 6.0- to 6.5-kb transcript and a minor, approximately 4.0-kb transcript. By PCR screening somatic cell hybrid DNAs and a YAC contig, Spicer et al. (1997) localized the human HAS3 gene to 16q22.1. By interspecific backcross analysis, they mapped the mouse Has3 gene to chromosome 8. Since HAS1 (OMIM Ref. No. 601463), HAS2 (OMIM Ref. No. 601636), and HAS3 are located on different autosomes, Spicer et al. (1997) suggested that the HAS gene family arose comparatively early in vertebrate evolution by sequential duplication of an ancestral HAS gene.

Full details of the abovementioned studies are described in the following publications, the disclosure of which are hereby incorporated by reference:
Spicer, A. P.; Olson, J. S.; McDonald, J. A.: Molecular cloning and characterization of a cDNA encoding the third putative mammalian hyaluronan synthase. J. Biol. Chem. 272: 8957-8961, 1997.; and
Spicer, A. P.; Seldin, M. F.; Olsen, A. S.; Brown, N.; Wells, D. E.; Doggett, N. A.; Itano, N.; Kimata, K.; Inazawa, J.; McDonald, J. A.: Chromosomal localization of the human and mous.

Further studies establishing the function and utilities of HAS3 are found in John Hopkins OMIM database record ID 602428, and in sited publications numbered 2011 listed in the bibliography section hereinbelow, which are also hereby incorporated by reference. V-jun Sarcoma Virus 17 Oncogene Homolog (avian) (JUN, Accession NM_{—}002228) is another VGAM224 host target gene. JUN BINDING SITE is HOST TARGET binding site found in the 3′ untranslated region of mRNA encoded by JUN, corresponding to a HOST TARGET binding site such as BINDING SITE I, BINDING SITE II or BINDING SITE III. Table 2 illustrates the complementarity of the nucleotide sequences of JUN BINDING SITE, designated SEQ ID:914, to the nucleotide sequence of VGAM224 RNA, herein designated VGAM RNA, also designated SEQ ID:559.

Another function of VGAM224 is therefore inhibition of V-jun Sarcoma Virus 17 Oncogene Homolog (avian) (JUN, Accession NM_{—}002228), a gene which binds and recognizes the enhancer dna sequencetga(c/g)tca. Accordingly, utilities of VGAM224 include diagnosis, prevention and treatment of diseases and clinical conditions associated with JUN. The function of JUN has been established by previous studies. The oncogene JUN is the putative transforming gene of avian sarcoma virus 17; it appears to be derived from a gene of the chicken genome and has homologs in several other vertebrate species. (The name JUN comes from the Japanese ‘ju-nana,’ meaning the number 17.) JUN was originally thought to be identical to the transcription factor AP1. However, it is now known that AP1 is not a single protein, but constitutes a group of related dimeric basic region-leucine zipper proteins that belong to the JUN, FOS (OMIM Ref. No. 164810), MAF (OMIM Ref. No. 177075), and ATF (see OMIM Ref. No. 603148) subfamilies. The various dimers recognize either 12-O-tetradecanoylphorbol-13-acetate (TPA) response elements or cAMP response elements. JUN is the most potent transcriptional activator in its group, and its transcriptional activity is attenuated and sometimes antagonized by JUNB (OMIM Ref. No. 165161). For a review of the structure and function of the AP1 transcription complexes Using a Drosophila model synapse, Sanyal et al. (2002) analyzed cellular functions and regulation of the immediate-early transcription factor AP1, a heterodimer of the basic leucine zipper proteins FOS and JUN. They observed that AP1 positively regulates synaptic strength and synapse number, thus showing a greater range of influence than CREB (OMIM Ref. No. 123810). Observations from genetic epistasis and RNA quantification experiments indicate that AP1 acts upstream of CREB, regulates levels of CREB mRNA, and functions at the top of the hierarchy of transcription factors known to regulate long-term plasticity. A JUN-kinase signaling module provided a CREB-independent route for neuronal AP1 activation; thus, CREB regulation of AP1 expression may, in some neurons, constitute a positive feedback loop rather than the primary step in AP1 activation. Mathas et al. (2002) found AP1 constitutively activated, with robust JUN and JUNB overexpression, in all cell lines derived from patients with classical Hodgkin lymphoma (OMIM Ref. No. 236000) and anaplastic large cell lymphoma (ALCL), but not in other lymphoma types. AP1 supported proliferation of Hodgkin cells, but suppressed apoptosis of ALCL cells. Mathas et al. (2002) noted that, whereas JUN is up-regulated by an autoregulatory process, JUNB is under the control of nuclear factor kappa-B (NFKB; 164011). They found that AP1 and NFKB cooperate and stimulate expression of the cell cycle regulator cyclin D2 (OMIM Ref. No. 123833), the protooncogene MET (OMIM Ref. No. 164860), and the lymphocyte homing receptor CCR7 (OMIM Ref. No. 600242), which are all strongly expressed in primary Hodgkin/Reed-Sternberg (HRS) cells.

Full details of the abovementioned studies are described in the following publications, the disclosure of which are hereby incorporated by reference:
Sanyal, S.; Sandstrom, D. J.; Hoeffer, C. A.; Ramaswami, M.: AP-1 function upstream of CREB to control synaptic plasticity in Drosophila. Nature 416: 870-874, 2002.; and
Mathas, S.; Hinz, M.; Anagnostopoulos, I.; Krappmann, D.; Lietz, A.; Jundt, F.; Bommert, K.; Mechta-Grigoriou, F.; Stein, H.; Dorken, B.; Scheidereit, C.: Aberrantly expressed c-Jun an.

Further studies establishing the function and utilities of JUN are found in John Hopkins OMIM database record ID 165160, and in sited publications numbered 1119, 1174-118 and 3060-1183 listed in the bibliography section hereinbelow, which are also hereby incorporated by reference. MAD, Mothers Against Decapentaplegic Homolog 9 (Drosophila) (MADH9, Accession NM_{—}005905) is another VGAM224 host target gene. MADH9 BINDING SITE is HOST TARGET binding site found in the 3′ untranslated region of mRNA encoded by MADH9, corresponding to a HOST TARGET binding site such as BINDING SITE I, BINDING SITE II or BINDING SITE III. Table 2 illustrates the complementarity of the nucleotide sequences of MADH9 BINDING SITE, designated SEQ ID:1260, to the nucleotide sequence of VGAM224 RNA, herein designated VGAM RNA, also designated SEQ ID:559.

Another function of VGAM224 is therefore inhibition of MAD, Mothers Against Decapentaplegic Homolog 9 (Drosophila) (MADH9, Accession NM_{—}005905). Accordingly, utilities of VGAM224 include diagnosis, prevention and treatment of diseases and clinical conditions associated with MADH9. Nuclear Receptor Sub family 2, Group C, Member 2 (NR2C2, Accession NM_{—}003298) is another VGAM224 host target gene. NR2C2 BINDING SITE is HOST TARGET binding site found in the 3′ untranslated region of mRNA encoded by NR2C2, corresponding to a HOST TARGET binding site such as BINDING SITE I, BINDING SITE II or BINDING SITE III. Table 2 illustrates the complementarity of the nucleotide sequences of NR2C2 BINDING SITE, designated SEQ ID: 1007, to the nucleotide sequence of VGAM224 RNA, herein designated VGAM RNA, also designated SEQ ID:559.

Another function of VGAM224 is therefore inhibition of Nuclear Receptor Sub family 2, Group C, Member 2 (NR2C2, Accession NM_{—}003298), a gene which may regulate gene expression during the late phase of spermatogenesis. Accordingly, utilities of VGAM224 include diagnosis, prevention and treatment of diseases and clinical conditions associated with NR2C2. The function of NR2C2 has been established by previous studies. Members of this class of so-called orphan receptors act as ligand-activated transcription factors. The proteins have an N-terminal transactivation domain, a central DNA-binding domain with 2 zinc fingers and a ligand-binding domain at the C terminus (Yoshikawa et al., 1996). The activated receptor/ligand complex is translocated to the nucleus where it binds to hormone response elements of target genes. Chang et al. (1994) cloned NR2C2, or TR4, a member of the nuclear hormone receptor super family, using degenerate PCR on RNA from the supraoptic nucleus of the brain with primers based on the conserved DNA-binding domain of these genes. They isolated TR4 cDNAs from both human and rat libraries. The cDNAs encode a predicted 615-amino acid human protein and a 596-amino acid rat protein that are 98% identical. The TR4 sequence is similar to that of the TR2 orphan receptor (Chang et al., 1994). Together they appear to form a distinct sub family. Hirose et al. (1994) cloned the TR4 gene (which they designated TAK1) from a human lymphoblastoma cDNA library. They stated that the predicted protein is 596 amino acids long. On SDS-PAGE, TR4 migrated as a 65-kD protein. Using Northern blot analysis, Hirose et al. (1994) found that TR4 is expressed as a 9.4-kb mRNA in many tissues, and as a 2.8-kb mRNA primarily in testis. The 2 transcripts appeared to differ in the length of the 3-prime untranslated region. In mouse and rat testis, TR4 was expressed most abundantly in spermatocytes.

Full details of the abovementioned studies are described in the following publications, the disclosure of which are hereby incorporated by reference:
Chang, C.; da Silva, S. L.; Ideta, R.; Lee, Y.; Yeh, S.; Burbach, J. P. H.: Human and rat TR4 orphan receptors specify a subclass of the steroid receptor super family. Proc. Nat. Acad. Sci. 91: 6040-6044, 1994.; and
Hirose, T.; Fujimoto, W.; Yamaai, T.; Kim, K. H.; Matsuura, H.; Jetten, A. M.: TAK1: Molecular cloning and characterization of a new member of the nuclear receptor super family. Molec. E.

Further studies establishing the function and utilities of NR2C2 are found in John Hopkins OMIM database record ID 601426, and in sited publications numbered 2086-2089 listed in the bibliography section hereinbelow, which are also hereby incorporated by reference. AP1S3 (Accession XM_{—}059421) is another VGAM224 host target gene. AP1S3 BINDING SITE is HOST TARGET binding site found in the 3′ untranslated region of mRNA encoded by AP1S3, corresponding to a HOST TARGET binding site such as BINDING SITE I, BINDING SITE II or BINDING SITE III. Table 2 illustrates the complementarity of the nucleotide sequences of AP1S3 BINDING SITE, designated SEQ ID:3010, to the nucleotide sequence of VGAM224 RNA, herein designated VGAM RNA, also designated SEQ ID:559.

Another function of VGAM224 is therefore inhibition of AP1S3 (Accession XM_{—}059421). Accordingly, utilities of VGAM224 include diagnosis, prevention and treatment of diseases and clinical conditions associated with AP1S3. Ras Homolog Gene Family, Member E (ARHE, Accession NM_{—}005168) is another VGAM224 host target gene. ARHE BINDING SITE is HOST TARGET binding site found in the 3′ untranslated region of mRNA encoded by ARHE, corresponding to a HOST TARGET binding site such as BINDING SITE I, BINDING SITE II or BINDING SITE III. Table 2 illustrates the complementarity of the nucleotide sequences of ARHE BINDING SITE, designated SEQ ID:1190, to the nucleotide sequence of VGAM224 RNA, herein designated VGAM RNA, also designated SEQ ID:559.

Another function of VGAM224 is therefore inhibition of Ras Homolog Gene Family, Member E (ARHE, Accession NM_{—}005168). Accordingly, utilities of VGAM224 include diagnosis, prevention and treatment of diseases and clinical conditions associated with ARHE. FLJ11000 (Accession NM_{—}018295) is another VGAM224 host target gene. FLJ11000 BINDING SITE is HOST TARGET binding site found in the 3′ untranslated region of mRNA encoded by FLJ11000, corresponding to a HOST TARGET binding site such as BINDING SITE I, BINDING SITE II or BINDING SITE III. Table 2 illustrates the complementarity of the nucleotide sequences of FLJ11000 BINDING SITE, designated SEQ ID:1812, to the nucleotide sequence of VGAM224 RNA, herein designated VGAM RNA, also designated SEQ ID:559.

Another function of VGAM224 is therefore inhibition of FLJ11000 (Accession NM_{—}018295). Accordingly, utilities of VGAM224 include diagnosis, prevention and treatment of diseases and clinical conditions associated with FLJ11000. FLJ11996 (Accession NM_{—}024976) is another VGAM224 host target gene. FLJ11996 BINDING SITE is HOST TARGET binding site found in the 5′ untranslated region of mRNA encoded by FLJ11996, corresponding to a HOST TARGET binding site such as BINDING SITE I, BINDING SITE II or BINDING SITE III. Table 2 illustrates the complementarity of the nucleotide sequences of FLJ11996 BINDING SITE, designated SEQ ID:2120, to the nucleotide sequence of VGAM224 RNA, herein designated VGAM RNA, also designated SEQ ID:559.

Another function of VGAM224 is therefore inhibition of FLJ11996 (Accession NM_{—}024976). Accordingly, utilities of VGAM224 include diagnosis, prevention and treatment of diseases and clinical conditions associated with FLJ11996. Growth Arrest and DNA-damage-inducible, Alpha (GADD45A, Accession NM_{—}001924) is another VGAM224 host target gene. GADD45A BINDING SITE is HOST TARGET binding site found in the 3′ untranslated region of mRNA encoded by GADD45A, corresponding to a HOST TARGET binding site such as BINDING SITE I, BINDING SITE II or BINDING SITE III. Table 2 illustrates the complementarity of the nucleotide sequences of GADD45A BINDING SITE, designated SEQ ID:869, to the nucleotide sequence of VGAM224 RNA, herein designated VGAM RNA, also designated SEQ ID:559.

Another function of VGAM224 is therefore inhibition of Growth Arrest and DNA-damage-inducible, Alpha (GADD45A, Accession NM_{—}001924). Accordingly, utilities of VGAM224 include diagnosis, prevention and treatment of diseases and clinical conditions associated with GADD45A. Heterogeneous Nuclear Ribonucleoprotein A3 (HNRPA3, Accession NM_{—}005758) is another VGAM224 host target gene. HNRPA3 BINDING SITE is HOST TARGET binding site found in the 3′ untranslated region of mRNA encoded by HNRPA3, corresponding to a HOST TARGET binding site such as BINDING SITE I, BINDING SITE II or BINDING SITE III. Table 2 illustrates the complementarity of the nucleotide sequences of HNRPA3 BINDING SITE, designated SEQ ID:1244, to the nucleotide sequence of VGAM224 RNA, herein designated VGAM RNA, also designated SEQ ID:559.

Another function of VGAM224 is therefore inhibition of Heterogeneous Nuclear Ribonucleoprotein A3 (HNRPA3, Accession NM_{—}005758). Accordingly, utilities of VGAM224 include diagnosis, prevention and treatment of diseases and clinical conditions associated with HNRPA3. KIAA0179 (Accession XM_{—}035973) is another VGAM224 host target gene. KIAA0179 BINDING SITE is HOST TARGET binding site found in the 3′ untranslated region of mRNA encoded by KIAA0179, corresponding to a HOST TARGET binding site such as BINDING SITE I, BINDING SITE II or BINDING SITE III. Table 2 illustrates the complementarity of the nucleotide sequences of KIAA0179 BINDING SITE, designated SEQ ID:2704, to the nucleotide sequence of VGAM224 RNA, herein designated VGAM RNA, also designated SEQ ID:559.

Another function of VGAM224 is therefore inhibition of KIAA0179 (Accession XM_{—}035973). Accordingly, utilities of VGAM224 include diagnosis, prevention and treatment of diseases and clinical conditions associated with KIAA0179. KIAA1831 (Accession XM_{—}033366) is another VGAM224 host target gene. KIAA1831 BINDING SITE is HOST TARGET binding site found in the 3′ untranslated region of mRNA encoded by KIAA1831, corresponding to a HOST TARGET binding site such as BINDING SITE I, BINDING SITE II or BINDING SITE III. Table 2 illustrates the complementarity of the nucleotide sequences of KIAA1831 BINDING SITE, designated SEQ ID:2667, to the nucleotide sequence of VGAM224 RNA, herein designated VGAM RNA, also designated SEQ ID:559.

Another function of VGAM224 is therefore inhibition of KIAA1831 (Accession XM_{—}033366). Accordingly, utilities of VGAM224 include diagnosis, prevention and treatment of diseases and clinical conditions associated with KIAA1831. Kelch-like 4 (Drosophila) (KLHL4, Accession NM_{—}019117) is another VGAM224 host target gene. KLHL4 BINDING SITE is HOST TARGET binding site found in the 3′ untranslated region of mRNA encoded by KLHL4, corresponding to a HOST TARGET binding site such as BINDING SITE I, BINDING SITE II or BINDING SITE III. Table 2 illustrates the complementarity of the nucleotide sequences of KLHL4 BINDING SITE, designated SEQ ID:1880, to the nucleotide sequence of VGAM224 RNA, herein designated VGAM RNA, also designated SEQ ID:559.

Another function of VGAM224 is therefore inhibition of Kelch-like 4 (Drosophila) (KLHL4, Accession NM_{—}019117). Accordingly, utilities of VGAM224 include diagnosis, prevention and treatment of diseases and clinical conditions associated with KLHL4. PANX3 (Accession NM_{—}052959) is another VGAM224 host target gene. PANX3 BINDING SITE is HOST TARGET binding site found in the 3′ untranslated region of mRNA encoded by PANX3, corresponding to a HOST TARGET binding site such as BINDING SITE I, BINDING SITE II or BINDING SITE III. Table 2 illustrates the complementarity of the nucleotide sequences of PANX3 BINDING SITE, designated SEQ ID:2350, to the nucleotide sequence of VGAM224 RNA, herein designated VGAM RNA, also designated SEQ ID:559.

Another function of VGAM224 is therefore inhibition of PANX3 (Accession NM_{—}052959). Accordingly, utilities of VGAM224 include diagnosis, prevention and treatment of diseases and clinical conditions associated with PANX3. TNF Receptor-associated Factor 3 (TRAF3, Accession XM_{—}007256) is another VGAM224 host target gene. TRAF3 BINDING SITE1 and TRAF3 BINDING SITE2 are HOST TARGET binding sites found in untranslated regions of mRNA encoded by TRAF3, corresponding to HOST TARGET binding sites such as BINDING SITE I, BINDING SITE II or BINDING SITE III. Table 2 illustrates the complementarity of the nucleotide sequences of TRAF3 BINDING SITE1 and TRAF3 BINDING SITE2, designated SEQ ID:2541 and SEQ ID:1008 respectively, to the nucleotide sequence of VGAM224 RNA, herein designated VGAM RNA, also designated SEQ ID:559.

Another function of VGAM224 is therefore inhibition of TNF Receptor-associated Factor 3 (TRAF3, Accession XM_{—}007256). Accordingly, utilities of VGAM224 include diagnosis, prevention and treatment of diseases and clinical conditions associated with TRAF3. ZID (Accession NM_{—}006626) is another VGAM224 host target gene. ZID BINDING SITE is HOST TARGET binding site found in the 3′ untranslated region of mRNA encoded by ZID, corresponding to a HOST TARGET binding site such as BINDING SITE I, BINDING SITE II or BINDING SITE III. Table 2 illustrates the complementarity of the nucleotide sequences of ZID BINDING SITE, designated SEQ ID:1315, to the nucleotide sequence of VGAM224 RNA, herein designated VGAM RNA, also designated SEQ ID:559.

Another function of VGAM224 is therefore inhibition of ZID (Accession NM_{—}006626). Accordingly, utilities of VGAM224 include diagnosis, prevention and treatment of diseases and clinical conditions associated with ZID. LOC151742 (Accession NM_{—}139245) is another VGAM224 host target gene. LOC151742 BINDING SITE is HOST TARGET binding site found in the 5′ untranslated region of mRNA encoded by LOC151742, corresponding to a HOST TARGET binding site such as BINDING SITE I, BINDING SITE II or BINDING SITE III. Table 2 illustrates the complementarity of the nucleotide sequences of LOC151742 BINDING SITE, designated SEQ ID:2477, to the nucleotide sequence of VGAM224 RNA, herein designated VGAM RNA, also designated SEQ ID:559.

Another function of VGAM224 is therefore inhibition of LOC151742 (Accession NM_{—}139245). Accordingly, utilities of VGAM224 include diagnosis, prevention and treatment of diseases and clinical conditions associated with LOC151742. LOC152179 (Accession XM_{—}098170) is another VGAM224 host target gene. LOC152179 BINDING SITE is HOST TARGET binding site found in the 3′ untranslated region of mRNA encoded by LOC152179, corresponding to a HOST TARGET binding site such as BINDING SITE I, BINDING SITE II or BINDING SITE III. Table 2 illustrates the complementarity of the nucleotide sequences of LOC152179 BINDING SITE, designated SEQ ID:3352, to the nucleotide sequence of VGAM224 RNA, herein designated VGAM RNA, also designated SEQ ID:559.

Another function of VGAM224 is therefore inhibition of LOC152179 (Accession XM_{—}098170). Accordingly, utilities of VGAM224 include diagnosis, prevention and treatment of diseases and clinical conditions associated with LOC152179. LOC51266 (Accession NM_{—}016509) is another VGAM224 host target gene. LOC51266 BINDING SITE is HOST TARGET binding site found in the 3′ untranslated region of mRNA encoded by LOC51266, corresponding to a HOST TARGET binding site such as BINDING SITE I, BINDING SITE II or BINDING SITE III. Table 2 illustrates the complementarity of the nucleotide sequences of LOC51266 BINDING SITE, designated SEQ ID:1691, to the nucleotide sequence of VGAM224 RNA, herein designated VGAM RNA, also designated SEQ ID:559.

Another function of VGAM224 is therefore inhibition of LOC51266 (Accession NM_{—}016509). Accordingly, utilities of VGAM224 include diagnosis, prevention and treatment of diseases and clinical conditions associated with LOC51266. LOC90317 (Accession XM_{—}030892) is another VGAM224 host target gene. LOC90317 BINDING SITE is HOST TARGET binding site found in the 5′ untranslated region of mRNA encoded by LOC90317, corresponding to a HOST TARGET binding site such as BINDING SITE I, BINDING SITE II or BINDING SITE III. Table 2 illustrates the complementarity of the nucleotide sequences of LOC90317 BINDING SITE, designated SEQ ID:2629, to the nucleotide sequence of VGAM224 RNA, herein designated VGAM RNA, also designated SEQ ID:559.

Another function of VGAM224 is therefore inhibition of LOC90317 (Accession XM_{—}030892). Accordingly, utilities of VGAM224 include diagnosis, prevention and treatment of diseases and clinical conditions associated with LOC90317. LOC90470 (Accession XM_{—}031975) is another VGAM224 host target gene. LOC90470 BINDING SITE is HOST TARGET binding site found in the 3′ untranslated region of mRNA encoded by LOC90470, corresponding to a HOST TARGET binding site such as BINDING SITE I, BINDING SITE II or BINDING SITE III. Table 2 illustrates the complementarity of the nucleotide sequences of LOC90470 BINDING SITE, designated SEQ ID:2645, to the nucleotide sequence of VGAM224 RNA, herein designated VGAM RNA, also designated SEQ ID:559.

Another function of VGAM224 is therefore inhibition of LOC90470 (Accession XM_{—}031975). Accordingly, utilities of VGAM224 include diagnosis, prevention and treatment of diseases and clinical conditions associated with LOC90470. LOC90786 (Accession XM_{—}034127) is another VGAM224 host target gene. LOC90786 BINDING SITE is HOST TARGET binding site found in the 5′ untranslated region of mRNA encoded by LOC90786, corresponding to a HOST TARGET binding site such as BINDING SITE I, BINDING SITE II or BINDING SITE III. Table 2 illustrates the complementarity of the nucleotide sequences of LOC90786 BINDING SITE, designated SEQ ID:2678, to the nucleotide sequence of VGAM224 RNA, herein designated VGAM RNA, also designated SEQ ID:559.

Another function of VGAM224 is therefore inhibition of LOC90786 (Accession XM_{—}034127). Accordingly, utilities of VGAM224 include diagnosis, prevention and treatment of diseases and clinical conditions associated with LOC90786. FIG. 1 further provides a conceptual description of a novel bioinformatically detected viral gene of the present invention, referred to here as Viral Genomic Address Messenger 225 (VGAM225) viral gene, which modulates expression of respective host target genes thereof, the function and utility of which host target genes is known in the art.

VGAM225 is a novel bioinformatically detected regulatory, non protein coding, viral micro RNA (miRNA) gene. The method by which VGAM225 was detected is described hereinabove with reference to FIGS. 1-8.

VGAM225 gene, herein designated VGAM GENE, is a viral gene contained in the genome of Vaccinia Virus. VGAM225 host target gene, herein designated VGAM HOST TARGET GENE, is a human gene contained in the human genome.

VGAM225 gene encodes a VGAM225 precursor RNA, herein designated VGAM PRECURSOR RNA. Similar to other miRNA genes, and unlike most ordinary genes, VGAM225 precursor RNA does not encode a protein. A nucleotide sequence identical or highly similar to the nucleotide sequence of VGAM225 precursor RNA is designated SEQ ID:211, and is provided hereinbelow with reference to the sequence listing part. Nucleotide sequence SEQ ID:211 is located at position 118961 relative to the genome of Vaccinia Virus.

VGAM225 precursor RNA folds onto itself, forming VGAM225 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM225 folded precursor RNA into VGAM225 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM225 RNA is designated SEQ ID:560, and is provided hereinbelow with reference to the sequence listing part.

VGAM225 host target gene, herein designated VGAM HOST TARGET GENE, encodes a corresponding messenger RNA, VGAM225 host target RNA, herein designated VGAM HOST TARGET RNA. VGAM225 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM225 RNA, herein designated VGAM RNA, binds complementarily to one or more host target binding sites located in untranslated regions of VGAM225 host target RNA, herein designated VGAM HOST TARGET RNA. This complementary binding is due to the fact that the nucleotide sequence of VGAM225 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM225 RNA, herein designated VGAM RNA, may have a different number of host target binding sites in untranslated regions of a VGAM225 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM225 RNA, herein designated VGAM RNA, to host target binding sites on VGAM225 host target RNA, herein designated VGAM HOST TARGET RNA, such as BINDING SITE I, BINDING SITE II and BINDING SITE III, inhibits translation of VGAM225 host target RNA into VGAM225 host target protein, herein designated VGAM HOST TARGET PROTEIN. VGAM host target protein is therefore outlined by a broken line.

It is appreciated that VGAM225 host target gene, herein designated VGAM HOST TARGET GENE, in fact represents a plurality of VGAM225 host target genes. The mRNA of each one of this plurality of VGAM225 host target genes comprises one or more host target binding sites, each having a nucleotide sequence which is at least partly complementary to VGAM225 RNA, herein designated VGAM RNA, and which when bound by VGAM225 RNA causes inhibition of translation of respective one or more VGAM225 host target proteins.

It is further appreciated by one skilled in the art that the mode of translational inhibition illustrated by FIG. 1 with specific reference to translational inhibition exerted by VGAM225 gene, herein designated VGAM GENE, on one or more VGAM225 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM225 is inhibition of expression of host target genes, as part of a novel viral mechanism of attacking a host. Accordingly, utilities of VGAM225 include diagnosis, prevention and treatment of viral infection by Vaccinia Virus. Specific functions, and accordingly utilities, of VGAM225 correlate with, and may be deduced from, the identity of the host target genes which VGAM225 binds and inhibits, and the function of these host target genes, as elaborated hereinbelow.

Nucleotide sequences of the VGAM225 precursor RNA, herein designated VGAM PRECURSOR RNA, and of the ‘diced’ VGAM225 RNA, herein designated VGAM RNA, and a schematic representation of the secondary folding of VGAM225 folded precursor RNA, herein designated VGAM FOLDED PRECURSOR RNA, of VGAM225 are further described hereinbelow with reference to Table 1.

Nucleotide sequences of host target binding sites, such as BINDING SITE-I, BINDING SITE-II and BINDING SITE-III of FIG. 1, found on VGAM225 host target RNA, and schematic representation of the complementarity of each of these host target binding sites to VGAM225 RNA, herein designated VGAM RNA, are described hereinbelow with reference to Table 2.

As mentioned hereinabove with reference to FIG. 1, a function of VGAM225 gene, herein designated VGAM is inhibition of expression of VGAM225 target genes. It is appreciated that specific functions, and accordingly utilities, of VGAM225 correlate with, and may be deduced from, the identity of the target genes which VGAM225 binds and inhibits, and the function of these target genes, as elaborated hereinbelow.

Alpha Thalassemia/mental Retardation Syndrome X-linked (RAD54 homolog, S. cerevisiae) (ATRX, Accession NM_{—}000489) is a VGAM225 host target gene. ATRX BINDING SITE1 and ATRX BINDING SITE2 are HOST TARGET binding sites found in untranslated regions of mRNA encoded by ATRX, corresponding to HOST TARGET binding sites such as BINDING SITE I, BINDING SITE II or BINDING SITE III. Table 2 illustrates the complementarity of the nucleotide sequences of ATRX BINDING SITE1 and ATRX BINDING SITE2, designated SEQ ID:751 and SEQ ID:2431 respectively, to the nucleotide sequence of VGAM225 RNA, herein designated VGAM RNA, also designated SEQ ID:560.

A function of VGAM225 is therefore inhibition of Alpha Thalassemia/mental Retardation Syndrome X-linked (RAD54 homolog, S. cerevisiae) (ATRX, Accession NM_{—}000489). Accordingly, utilities of VGAM225 include diagnosis, prevention and treatment of diseases and clinical conditions associated with ATRX. RAB27A, Member RAS Oncogene Family (RAB27A, Accession NM_{—}004580) is another VGAM225 host target gene. RAB27A BINDING SITE is HOST TARGET binding site found in the 3′ untranslated region of mRNA encoded by RAB27A, corresponding to a HOST TARGET binding site such as BINDING SITE I, BINDING SITE II or BINDING SITE III. Table 2 illustrates the complementarity of the nucleotide sequences of RAB27A BINDING SITE, designated SEQ ID:1128, to the nucleotide sequence of VGAM225 RNA, herein designated VGAM RNA, also designated SEQ ID:560.

Another function of VGAM225 is therefore inhibition of RAB27A, Member RAS Oncogene Family (RAB27A, Accession NM_{—}004580). Accordingly, utilities of VGAM225 include diagnosis, prevention and treatment of diseases and clinical conditions associated with RAB27A. Cylindromatosis (turban tumor syndrome) (CYLD, Accession NM_{—}015247) is another VGAM225 host target gene. CYLD BINDING SITE is HOST TARGET binding site found in the 3′ untranslated region of mRNA encoded by CYLD, corresponding to a HOST TARGET binding site such as BINDING SITE I, BINDING SITE II or BINDING SITE III. Table 2 illustrates the complementarity of the nucleotide sequences of CYLD BINDING SITE, designated SEQ ID: 1616, to the nucleotide sequence of VGAM225 RNA, herein designated VGAM RNA, also designated SEQ ID:560.

Another function of VGAM225 is therefore inhibition of Cylindromatosis (turban tumor syndrome) (CYLD, Accession NM_{—}015247). Accordingly, utilities of VGAM225 include diagnosis, prevention and treatment of diseases and clinical conditions associated with CYLD. LOC145945 (Accession XM_{—}096908) is another VGAM225 host target gene. LOC145945 BINDING SITE is HOST TARGET binding site found in the 5′ untranslated region of mRNA encoded by LOC145945, corresponding to a HOST TARGET binding site such as BINDING SITE I, BINDING SITE II or BINDING SITE III. Table 2 illustrates the complementarity of the nucleotide sequences of LOC145945 BINDING SITE, designated SEQ ID:3279, to the nucleotide sequence of VGAM225 RNA, herein designated VGAM RNA, also designated SEQ ID:560.

Another function of VGAM225 is therefore inhibition of LOC145945 (Accession XM_{—}096908). Accordingly, utilities of VGAM225 include diagnosis, prevention and treatment of diseases and clinical conditions associated with LOC145945. LOC200273 (Accession XM_{—}047698) is another VGAM225 host target gene. LOC200273 BINDING SITE is HOST TARGET binding site found in the 3′ untranslated region of mRNA encoded by LOC200273, corresponding to a HOST TARGET binding site such as BINDING SITE I, BINDING SITE II or BINDING SITE III. Table 2 illustrates the complementarity of the nucleotide sequences of LOC200273 BINDING SITE, designated SEQ ID:2895, to the nucleotide sequence of VGAM225 RNA, herein designated VGAM RNA, also designated SEQ ID:560.

Another function of VGAM225 is therefore inhibition of LOC200273 (Accession XM_{—}047698). Accordingly, utilities of VGAM225 include diagnosis, prevention and treatment of diseases and clinical conditions associated with LOC200273. LOC220758 (Accession XM_{—}165466) is another VGAM225 host target gene. LOC220758 BINDING SITE is HOST TARGET binding site found in the 3′ untranslated region of mRNA encoded by LOC220758, corresponding to a HOST TARGET binding site such as BINDING SITE I, BINDING SITE II or BINDING SITE III. Table 2 illustrates the complementarity of the nucleotide sequences of LOC220758 BINDING SITE, designated SEQ ID:3497, to the nucleotide sequence of VGAM225 RNA, herein designated VGAM RNA, also designated SEQ ID:560.

Another function of VGAM225 is therefore inhibition of LOC220758 (Accession XM_{—}165466). Accordingly, utilities of VGAM225 include diagnosis, prevention and treatment of diseases and clinical conditions associated with LOC220758. FIG. 1 further provides a conceptual description of a novel bioinformatically detected viral gene of the present invention, referred to here as Viral Genomic Address Messenger 226 (VGAM226) viral gene, which modulates expression of respective host target genes thereof, the function and utility of which host target genes is known in the art.

VGAM226 is a novel bioinformatically detected regulatory, non protein coding, viral micro RNA (miRNA) gene. The method by which VGAM226 was detected is described hereinabove with reference to FIGS. 1-8.

VGAM226 gene, herein designated VGAM GENE, is a viral gene contained in the genome of Vaccinia Virus. VGAM226 host target gene, herein designated VGAM HOST TARGET GENE, is a human gene contained in the human genome.

VGAM226 gene encodes a VGAM226 precursor RNA, herein designated VGAM PRECURSOR RNA. Similar to other miRNA genes, and unlike most ordinary genes, VGAM226 precursor RNA does not encode a protein. A nucleotide sequence identical or highly similar to the nucleotide sequence of VGAM226 precursor RNA is designated SEQ ID:212, and is provided hereinbelow with reference to the sequence listing part. Nucleotide sequence SEQ ID:212 is located at position 118120 relative to the genome of Vaccinia Virus.

VGAM226 precursor RNA folds onto itself, forming VGAM226 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM226 folded precursor RNA into VGAM226 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM226 RNA is designated SEQ ID:561, and is provided hereinbelow with reference to the sequence listing part.

VGAM226 host target gene, herein designated VGAM HOST TARGET GENE, encodes a corresponding messenger RNA, VGAM226 host target RNA, herein designated VGAM HOST TARGET RNA. VGAM226 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM226 RNA, herein designated VGAM RNA, binds complementarily to one or more host target binding sites located in untranslated regions of VGAM226 host target RNA, herein designated VGAM HOST TARGET RNA. This complementary binding is due to the fact that the nucleotide sequence of VGAM226 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM226 RNA, herein designated VGAM RNA, may have a different number of host target binding sites in untranslated regions of a VGAM226 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM226 RNA, herein designated VGAM RNA, to host target binding sites on VGAM226 host target RNA, herein designated VGAM HOST TARGET RNA, such as BINDING SITE I, BINDING SITE II and BINDING SITE III, inhibits translation of VGAM226 host target RNA into VGAM226 host target protein, herein designated VGAM HOST TARGET PROTEIN. VGAM host target protein is therefore outlined by a broken line.

It is appreciated that VGAM226 host target gene, herein designated VGAM HOST TARGET GENE, in fact represents a plurality of VGAM226 host target genes. The mRNA of each one of this plurality of VGAM226 host target genes comprises one or more host target binding sites, each having a nucleotide sequence which is at least partly complementary to VGAM226 RNA, herein designated VGAM RNA, and which when bound by VGAM226 RNA causes inhibition of translation of respective one or more VGAM226 host target proteins.

It is further appreciated by one skilled in the art that the mode of translational inhibition illustrated by FIG. 1 with specific reference to translational inhibition exerted by VGAM226 gene, herein designated VGAM GENE, on one or more VGAM226 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM226 is inhibition of expression of host target genes, as part of a novel viral mechanism of attacking a host. Accordingly, utilities of VGAM226 include diagnosis, prevention and treatment of viral infection by Vaccinia Virus. Specific functions, and accordingly utilities, of VGAM226 correlate with, and may be deduced from, the identity of the host target genes which VGAM226 binds and inhibits, and the function of these host target genes, as elaborated hereinbelow.

Nucleotide sequences of the VGAM226 precursor RNA, herein designated VGAM PRECURSOR RNA, and of the ‘diced’ VGAM226 RNA, herein designated VGAM RNA, and a schematic representation of the secondary folding of VGAM226 folded precursor RNA, herein designated VGAM FOLDED PRECURSOR RNA, of VGAM226 are further described hereinbelow with reference to Table 1.

Nucleotide sequences of host target binding sites, such as BINDING SITE-I, BINDING SITE-II and BINDING SITE-III of FIG. 1, found on VGAM226 host target RNA, and schematic representation of the complementarity of each of these host target binding sites to VGAM226 RNA, herein designated VGAM RNA, are described hereinbelow with reference to Table 2.

As mentioned hereinabove with reference to FIG. 1, a function of VGAM226 gene, herein designated VGAM is inhibition of expression of VGAM226 target genes. It is appreciated that specific functions, and accordingly utilities, of VGAM226 correlate with, and may be deduced from, the identity of the target genes which VGAM226 binds and inhibits, and the function of these target genes, as elaborated hereinbelow.

Kinesin-like 1 (KNSL1, Accession NM_{—}004523) is a VGAM226 host target gene. KNSL1 BINDING SITE is HOST TARGET binding site found in the 3′ untranslated region of mRNA encoded by KNSL1, corresponding to a HOST TARGET binding site such as BINDING SITE I, BINDING SITE II or BINDING SITE III. Table 2 illustrates the complementarity of the nucleotide sequences of KNSL1 BINDING SITE, designated SEQ ID:1123, to the nucleotide sequence of VGAM226 RNA, herein designated VGAM RNA, also designated SEQ ID:561.

A function of VGAM226 is therefore inhibition of Kinesin-like 1 (KNSL1, Accession NM_{—}004523), a gene which is a motor protein required for establishing a bipolar spindle. Accordingly, utilities of VGAM226 include diagnosis, prevention and treatment of diseases and clinical conditions associated with KNSL1. The function of KNSL1 has been established by previous studies. Kinesins are tubulin (see OMIM Ref. No. 191130) molecular motors that function to transport organelles within cells and to move chromosomes along microtubules during cell division. In sea urchin and mammalian cells, kinesins have been characterized as tetrameric proteins comprising 2 heavy chains (alpha chains) of approximately 120 kD and 2 light chains (beta chains) of approximately 70 kD. The alpha chains provide the tubulin binding site and the ATPase domains, whereas the beta chains are responsible for the specific attachment of the organelle to be moved by the kinesin tetramer. Kinesins transport their bound organelle to the plus end of the microtubule. Chernajovsky et al., (1996) noted that differential splicing occurs for the kinesin beta (light) cDNA sequences at the 3-prime end of the rat kinesin mRNA, producing kinesins having different C-terminal ends that seem to confer the kinesin specificity for organelle binding. Cabeza-Arvelaiz et al. (1993) isolated and sequenced a cDNA encoding the human kinesin light chain protein (KLC). The cDNA consists of 276 nucleotides of 5-prime untranslated region, a coding sequence of 1,710 nucleotides, and 322 nucleotides of 3-prime untranslated region. It encodes a polypeptide of 569 amino acids and a deduced molecular mass of 64,789 daltons. The predicted secondary internal structure of the KLC molecule consists of about 27 contiguous repeats, each of approximately 21 amino acids, and could be divided into 3 domains. See also 601334 Chernajovsky et al. (1996) characterized the human KNS2 gene product of a differentially spliced, T-cell-derived mRNA and cloned its promoter region. The promoter region transcribes constitutively. In permanently transfected human HeLa and NB100 neuroblastoma cells, a reporter gene containing the promoter and part of the first exon of beta kinesin was 75-fold more active than the HSV-tk promoter. The first exon contains a 5-prime untranslated sequence capable of forming a stable double-hairpin loop, which functions as a translational enhancer. Its deletion decreases the efficiency of in vitro translation of beta kinesin mRNA. Kamal et al. (2000) demonstrated that the axonal transport of APP (OMIM Ref. No. 104760) in neurons is mediated by the direct binding of APP to the kinesin light chain subunit of kinesin-1.

Full details of the abovementioned studies are described in the following publications, the disclosure of which are hereby incorporated by reference:
Goedert, M.; Marsh, S.; Carter, N.: Localization of the human kinesin light chain gene (KNS2) to chromosome 14q32.3 by fluorescence in situ hybridization. Genomics 32: 173-175, 1996.; and
Kamal, A.; Stokin, G. B.; Yang, Z.; Xia, C.; Goldstein, L. S.: Axonal transport of amyloid precursor protein is mediated by direct binding to the kinesin light chain subunit of kinesin-.

Further studies establishing the function and utilities of KNSL1 are found in John Hopkins OMIM database record ID 148760, and in sited publications numbered 72 and 2790-724 listed in the bibliography section hereinbelow, which are also hereby incorporated by reference. Placenta-specific 1 (PLAC1, Accession NM_{—}021796) is another VGAM226 host target gene. PLAC1 BINDING SITE is HOST TARGET binding site found in the 3′ untranslated region of mRNA encoded by PLAC1, corresponding to a HOST TARGET binding site such as BINDING SITE I, BINDING SITE II or BINDING SITE III. Table 2 illustrates the complementarity of the nucleotide sequences of PLAC1 BINDING SITE, designated SEQ ID:1960, to the nucleotide sequence of VGAM226 RNA, herein designated VGAM RNA, also designated SEQ ID:561.

Another function of VGAM226 is therefore inhibition of Placenta-specific 1 (PLAC1, Accession NM_{—}021796). Accordingly, utilities of VGAM226 include diagnosis, prevention and treatment of diseases and clinical conditions associated with PLAC1. Sterol Carrier Protein 2 (SCP2, Accession NM_{—}002979) is another VGAM226 host target gene. SCP2 BINDING SITE is HOST TARGET binding site found in the 3′ untranslated region of mRNA encoded by SCP2, corresponding to a HOST TARGET binding site such as BINDING SITE I, BINDING SITE II or BINDING SITE III. Table 2 illustrates the complementarity of the nucleotide sequences of SCP2 BINDING SITE, designated SEQ ID:977, to the nucleotide sequence of VGAM226 RNA, herein designated VGAM RNA, also designated SEQ ID:561.

Another function of VGAM226 is therefore inhibition of Sterol Carrier Protein 2 (SCP2, Accession NM_{—}002979), a gene which may regulate steroidogenesis. Accordingly, utilities of VGAM226 include diagnosis, prevention and treatment of diseases and clinical conditions associated with SCP2. The function of SCP2 and its association with various diseases and clinical conditions, has been established by previous studies, as described hereinabove with reference to VGAM191. Stromal Interaction Molecule 1 (STIM1, Accession XM_{—}011967) is another VGAM226 host target gene. STIM1 BINDING SITE is HOST TARGET binding site found in the 3′ untranslated region of mRNA encoded by STIM1, corresponding to a HOST TARGET binding site such as BINDING SITE I, BINDING SITE II or BINDING SITE III. Table 2 illustrates the complementarity of the nucleotide sequences of STIM1 BINDING SITE, designated SEQ ID:2558, to the nucleotide sequence of VGAM226 RNA, herein designated VGAM RNA, also designated SEQ ID:561.

Another function of VGAM226 is therefore inhibition of Stromal Interaction Molecule 1 (STIM1, Accession XM_{—}011967), a gene which is very strongly similar to murine Stim1 and may be a transmembrane stromal cell protein. Accordingly, utilities of VGAM226 include diagnosis, prevention and treatment of diseases and clinical conditions associated with STIM1. The function of STIM1 has been established by previous studies. Using sequences identified by database searching with a transcript from human chromosome 11p15.5, Parker et al. (1996) screened placental and fetal liver cDNA libraries and cloned a novel cDNA, STIM1, which they called GOK. The deduced 746-amino acid protein contains a predicted signal peptide and transmembrane helix. Parker et al. (1996) also cloned a partial mouse Stim1 genomic clone and found that the human and mouse proteins share 90% sequence identity. Restriction mapping by pulsed field electrophoresis placed the STIM1 gene 1.7 kb telomeric of the RRM1 gene (OMIM Ref. No. 180410) on 11p15.5 (Parker et al., 1996). Sabbioni et al. (1999) determined that the STIM1 gene contains 12 exons that span more than 250 kb between the RRM1 and NUP98 (OMIM Ref. No. 601021) genes.

Full details of the abovementioned studies are described in the following publications, the disclosure of which are hereby incorporated by reference:
Parker, N. J.; Begley, C. G.; Smith, P. J.; Fox, R. M.: Molecular cloning of a novel human gene (D11S4896E) at chromosomal region 11p15.5. Genomics 37: 253-256, 1996.; and
Sabbioni, S.; Veronese, A.; Trubia, M.; Taramelli, R.; Barbanti-Brodano, G.; Croce, C. M.; Negrini, M.: Exon structure and promoter identification of STIM1 (alias GOK), a human gene ca.

Further studies establishing the function and utilities of STIM1 are found in John Hopkins OMIM database record ID 605921, and in sited publications numbered 1496-1497 listed in the bibliography section hereinbelow, which are also hereby incorporated by reference. Cortactin Binding Protein 2 (CORTBP2, Accession NM_{—}033427) is another VGAM226 host target gene. CORTBP2 BINDING SITE is HOST TARGET binding site found in the 3′ untranslated region of mRNA encoded by CORTBP2, corresponding to a HOST TARGET binding site such as BINDING SITE I, BINDING SITE II or BINDING SITE III. Table 2 illustrates the complementarity of the nucleotide sequences of CORTBP2 BINDING SITE, designated SEQ ID:2334, to the nucleotide sequence of VGAM226 RNA, herein designated VGAM RNA, also designated SEQ ID:561.

Another function of VGAM226 is therefore inhibition of Cortactin Binding Protein 2 (CORTBP2, Accession NM_{—}033427). Accordingly, utilities of VGAM226 include diagnosis, prevention and treatment of diseases and clinical conditions associated with CORTBP2. Vesicle-associated Membrane Protein 3 (cellubrevin) (VAMP3, Accession NM_{—}004781) is another VGAM226 host target gene. VAMP3 BINDING SITE is HOST TARGET binding site found in the 3′ untranslated region of mRNA encoded by VAMP3, corresponding to a HOST TARGET binding site such as BINDING SITE I, BINDING SITE II or BINDING SITE III. Table 2 illustrates the complementarity of the nucleotide sequences of VAMP3 BINDING SITE, designated SEQ ID:1156, to the nucleotide sequence of VGAM226 RNA, herein designated VGAM RNA, also designated SEQ ID:561.

Another function of VGAM226 is therefore inhibition of Vesicle-associated Membrane Protein 3 (cellubrevin) (VAMP3, Accession NM_{—}004781). Accordingly, utilities of VGAM226 include diagnosis, prevention and treatment of diseases and clinical conditions associated with VAMP3. LOC90148 (Accession XM_{—}029430) is another VGAM226 host target gene. LOC90148 BINDING SITE is HOST TARGET binding site found in the 3′ untranslated region of mRNA encoded by LOC90148, corresponding to a HOST TARGET binding site such as BINDING SITE I, BINDING SITE II or BINDING SITE III. Table 2 illustrates the complementarity of the nucleotide sequences of LOC90148 BINDING SITE, designated SEQ ID:2603, to the nucleotide sequence of VGAM226 RNA, herein designated VGAM RNA, also designated SEQ ID:561.

Another function of VGAM226 is therefore inhibition of LOC90148 (Accession XM_{—}029430). Accordingly, utilities of VGAM226 include diagnosis, prevention and treatment of diseases and clinical conditions associated with LOC90148. FIG. 1 further provides a conceptual description of a novel bioinformatically detected viral gene of the present invention, referred to here as Viral Genomic Address Messenger 227 (VGAM227) viral gene, which modulates expression of respective host target genes thereof, the function and utility of which host target genes is known in the art.

VGAM227 is a novel bioinformatically detected regulatory, non protein coding, viral micro RNA (miRNA) gene. The method by which VGAM227 was detected is described hereinabove with reference to FIGS. 1-8.

VGAM227 gene, herein designated VGAM GENE, is a viral gene contained in the genome of Vaccinia Virus. VGAM227 host target gene, herein designated VGAM HOST TARGET GENE, is a human gene contained in the human genome.

VGAM227 gene encodes a VGAM227 precursor RNA, herein designated VGAM PRECURSOR RNA. Similar to other miRNA genes, and unlike most ordinary genes, VGAM227 precursor RNA does not encode a protein. A nucleotide sequence identical or highly similar to the nucleotide sequence of VGAM227 precursor RNA is designated SEQ ID:213, and is provided hereinbelow with reference to the sequence listing part. Nucleotide sequence SEQ ID:213 is located at position 118560 relative to the genome of Vaccinia Virus.

VGAM227 precursor RNA folds onto itself, forming VGAM227 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM227 folded precursor RNA into VGAM227 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM227 RNA is designated SEQ ID:562, and is provided hereinbelow with reference to the sequence listing part.

VGAM227 host target gene, herein designated VGAM HOST TARGET GENE, encodes a corresponding messenger RNA, VGAM227 host target RNA, herein designated VGAM HOST TARGET RNA. VGAM227 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM227 RNA, herein designated VGAM RNA, binds complementarily to one or more host target binding sites located in untranslated regions of VGAM227 host target RNA, herein designated VGAM HOST TARGET RNA. This complementary binding is due to the fact that the nucleotide sequence of VGAM227 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM227 RNA, herein designated VGAM RNA, may have a different number of host target binding sites in untranslated regions of a VGAM227 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM227 RNA, herein designated VGAM RNA, to host target binding sites on VGAM227 host target RNA, herein designated VGAM HOST TARGET RNA, such as BINDING SITE I, BINDING SITE II and BINDING SITE III, inhibits translation of VGAM227 host target RNA into VGAM227 host target protein, herein designated VGAM HOST TARGET PROTEIN. VGAM host target protein is therefore outlined by a broken line.

It is appreciated that VGAM227 host target gene, herein designated VGAM HOST TARGET GENE, in fact represents a plurality of VGAM227 host target genes. The mRNA of each one of this plurality of VGAM227 host target genes comprises one or more host target binding sites, each having a nucleotide sequence which is at least partly complementary to VGAM227 RNA, herein designated VGAM RNA, and which when bound by VGAM227 RNA causes inhibition of translation of respective one or more VGAM227 host target proteins.

It is further appreciated by one skilled in the art that the mode of translational inhibition illustrated by FIG. 1 with specific reference to translational inhibition exerted by VGAM227 gene, herein designated VGAM GENE, on one or more VGAM227 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM227 is inhibition of expression of host target genes, as part of a novel viral mechanism of attacking a host. Accordingly, utilities of VGAM227 include diagnosis, prevention and treatment of viral infection by Vaccinia Virus. Specific functions, and accordingly utilities, of VGAM227 correlate with, and may be deduced from, the identity of the host target genes which VGAM227 binds and inhibits, and the function of these host target genes, as elaborated hereinbelow.

Nucleotide sequences of the VGAM227 precursor RNA, herein designated VGAM PRECURSOR RNA, and of the ‘diced’ VGAM227 RNA, herein designated VGAM RNA, and a schematic representation of the secondary folding of VGAM227 folded precursor RNA, herein designated VGAM FOLDED PRECURSOR RNA, of VGAM227 are further described hereinbelow with reference to Table 1.

Nucleotide sequences of host target binding sites, such as BINDING SITE-I, BINDING SITE-II and BINDING SITE-III of FIG. 1, found on VGAM227 host target RNA, and schematic representation of the complementarity of each of these host target binding sites to VGAM227 RNA, herein designated VGAM RNA, are described hereinbelow with reference to Table 2.

As mentioned hereinabove with reference to FIG. 1, a function of VGAM227 gene, herein designated VGAM is inhibition of expression of VGAM227 target genes. It is appreciated that specific functions, and accordingly utilities, of VGAM227 correlate with, and may be deduced from, the identity of the target genes which VGAM227 binds and inhibits, and the function of these target genes, as elaborated hereinbelow.

Adenylate Kinase 2 (AK2, Accession NM_{—}013411) is a VGAM227 host target gene. AK2 BINDING SITE is HOST TARGET binding site found in the 3′ untranslated region of mRNA encoded by AK2, corresponding to a HOST TARGET binding site such as BINDING SITE I, BINDING SITE II or BINDING SITE III. Table 2 illustrates the complementarity of the nucleotide sequences of AK2 BINDING SITE, designated SEQ ID: 1449, to the nucleotide sequence of VGAM227 RNA, herein designated VGAM RNA, also designated SEQ ID:562.

A function of VGAM227 is therefore inhibition of Adenylate Kinase 2 (AK2, Accession NM_{—}013411), a gene which essential for maintenance and cell growth. Accordingly, utilities of VGAM227 include diagnosis, prevention and treatment of diseases and clinical conditions associated with AK2. The function of AK2 has been established by previous studies. The existence of a second adenylate kinase (EC 2.7.4.3) locus linked to PGM1 and peptidase C, i.e., on chromosome 1, was suggested by cell hybridization studies by Van Cong et al. (1972). The Goss-Harris method of mapping combines features of recombinational study in families and synteny tests in hybrid cells. As applied to chromosome 1, the method shows that AK2 and UMPK are distal to PGM1 and that the order of the loci is PGM1: UMPK: (AK2, alpha-FUC): ENO1 (Goss and Harris, 1977). Carritt et al. (1982) presented evidence that AK2 is in 1p34.

Full details of the abovementioned studies are described in the following publications, the disclosure of which are hereby incorporated by reference:
Goss, S. J.; Harris, H.: Gene transfer by means of cell fusion. II. The mapping of 8 loci on human chromosome 1 by statistical analysis of gene assortment in somatic cell hybrids. J. Cell Sci. 25: 39-57, 1977.; and
Carritt, B.; King, J.; Welch, H. M.: Gene order and localization of enzyme loci on the short arm of chromosome 1. Ann. Hum. Genet. 46: 329-335, 1982.

Further studies establishing the function and utilities of AK2 are found in John Hopkins OMIM database record ID 103020, and in sited publications numbered 188-191 listed in the bibliography section hereinbelow, which are also hereby incorporated by reference. Cartilage Associated Protein (CRTAP, Accession NM_{—}006371) is another VGAM227 host target gene. CRTAP BINDING SITE is HOST TARGET binding site found in the 3′ untranslated region of mRNA encoded by CRTAP, corresponding to a HOST TARGET binding site such as BINDING SITE I, BINDING SITE II or BINDING SITE III. Table 2 illustrates the complementarity of the nucleotide sequences of CRTAP BINDING SITE, designated SEQ ID:1291, to the nucleotide sequence of VGAM227 RNA, herein designated VGAM RNA, also designated SEQ ID:562.

Another function of VGAM227 is therefore inhibition of Cartilage Associated Protein (CRTAP, Accession NM_{—}006371), a gene which is a novel developmentally regulated chick embryo protein. Accordingly, utilities of VGAM227 include diagnosis, prevention and treatment of diseases and clinical conditions associated with CRTAP. The function of CRTAP has been established by previous studies. Castagnola et al. (1997) isolated a mouse Crtap cDNA from a subtracted library specific for mRNAs highly expressed in hypertrophic chondrocytes compared to proliferating and early differentiating chondrocytes. Using a mouse Crtap clone to screen a human fetal brain cDNA library, Tonachini et al. (1999) identified human CRTAP cDNA clones. Human CRTAP encodes a deduced 401-amino acid protein with a putative signal peptide of 26 amino acids. CRTAP contains 2 potential N-glycosylation signals. CRTAP shares 89% amino acid sequence identity with mouse Crtap and 51% identity with the chick homolog. The mouse and human genes contain a C-terminal region of approximately 120 amino acids not present in the chick protein Using Northern blot analysis of human tissues, Tonachini et al. (1999) detected 2-kb and 4-kb CRTAP transcripts in brain, heart, kidney, lung, small intestine, and skeletal muscle. In all tissues except brain, the 2-kb transcript was more abundant. Using immunohistochemistry, the authors detected CRTAP expression in articular chondrocytes. In mouse, Morello et al. (1999) detected 3 Crtap transcripts in a range of tissues, including all mouse embryonic cartilages. In chick, Castagnola et al. (1997) detected a single Crtap transcript in a broad range of embryonic tissues with the strongest expression in the developing cartilage. They detected expression in the extracellular matrix of the forming cartilage surrounding the notochord, the developing sclera, the sphenoid and mandibular cartilage, the long bone cartilage, and the developing sternal cartilage

Full details of the abovementioned studies are described in the following publications, the disclosure of which are hereby incorporated by reference:
Castagnola, P.; Gennari, M.; Morello, R.; Tonachini, L.; Marin, O.; Gaggero, A.; Cancedda, R.: Cartilage associated protein (CASP) is a novel developmentally regulated chick embryo protein. J. Cell Sci. 110: 1351-1359, 1997.; and
Morello, R.; Tonachini, L.; Monticone, M.; Viggiano, L.; Rocchi, M.; Cancedda, R.; Castagnola, P.: cDNA cloning, characterization and chromosome mapping of Crtap encoding the mouse carti.

Further studies establishing the function and utilities of CRTAP are found in John Hopkins OMIM database record ID 605497, and in sited publications numbered 1486-1488 listed in the bibliography section hereinbelow, which are also hereby incorporated by reference. Chromosome X Open Reading Frame 6 (CXorf6, Accession NM_{—}005491) is another VGAM227 host target gene. CXorf6 BINDING SITE is HOST TARGET binding site found in the 5′ untranslated region of mRNA encoded by CXorf6, corresponding to a HOST TARGET binding site such as BINDING SITE I, BINDING SITE II or BINDING SITE III. Table 2 illustrates the complementarity of the nucleotide sequences of CXorf6 BINDING SITE, designated SEQ ID:1218, to the nucleotide sequence of VGAM227 RNA, herein designated VGAM RNA, also designated SEQ ID:562.

Another function of VGAM227 is therefore inhibition of Chromosome X Open Reading Frame 6 (CXorf6, Accession NM_{—}005491). Accordingly, utilities of VGAM227 include diagnosis, prevention and treatment of diseases and clinical conditions associated with CXorf6. EphB2 (EPHB2, Accession NM_{—}004442) is another VGAM227 host target gene. EPHB2 BINDING SITE is HOST TARGET binding site found in the 3′ untranslated region of mRNA encoded by EPHB2, corresponding to a HOST TARGET binding site such as BINDING SITE I, BINDING SITE II or BINDING SITE III. Table 2 illustrates the complementarity of the nucleotide sequences of EPHB2 BINDING SITE, designated SEQ ID:1113, to the nucleotide sequence of VGAM227 RNA, herein designated VGAM RNA, also designated SEQ ID:562.

Another function of VGAM227 is therefore inhibition of EphB2 (EPHB2, Accession NM_{—}004442), a gene which Eph-related receptor tyrosine kinase B2; may have a role in neurogenesis. Accordingly, utilities of VGAM227 include diagnosis, prevention and treatment of diseases and clinical conditions associated with EPHB2. The function of EPHB2 has been established by previous studies. See 179610 for background on Eph receptors and their ligands, the ephrins. Chan and Watt (1991) cloned partial sequences of the EEK (EPHA8; 176945) and ERK genes encoding members of the EPH subclass of receptor protein-tyrosine kinases. Northern blot analysis of rat RNA showed that DNA encoding human ERK hybridized to transcripts most abundantly in lung. By screening a human fetal brain cDNA expression library using a monoclonal antiphospho-tyrosine antibody and by 5-prime RACE (rapid amplification of cDNA ends) procedures, Ikegaki et al. (1995) isolated overlapping cDNAs encoding a receptor-type tyrosine kinase belonging to the EPH family and designated the gene DRT (for developmentally regulated EPH-related tyrosine kinase). The DRT gene is expressed in transcripts of 3 different sizes (4, 5, and 11 kb). The DRT transcripts are expressed in human brain and several other tissues, including heart, lung, kidney, placenta, pancreas, liver, and skeletal muscle, but the 11-kb DRT transcript is preferentially expressed in fetal brain. Steady-state levels of DRT mRNA in several tissues, including brain, heart, lung, and kidney, are greater in the midterm fetus than those in the adult. Ikegaki et al. (1995) showed that a large number of tumor cell lines derived from neuroectoderm express DRT transcripts. The authors speculated that DRT may play a part in human neurogenesis. Using a yeast 2-hybrid system, Cowan et al. (2000) demonstrated that PDZ domain-containing protein Pick1 (PRKCABP; 605926) binds the C-terminal tail of EphB2. Using colocalization studies and biochemical analysis, they demonstrated that a protein complex containing EphB2 and aquaporin-1 (AQP1; 107776) is formed in vivo. They concluded that Ephb2 may regulate ionic homeostasis and endolymph fluid production through macromolecular associations with membrane channels that transport chloride, bicarbonate, and water. Chan and Watt (1991) mapped the EEK and ERK genes to chromosome 1 by Southern blot analysis of somatic cell hybrids. Ikegaki et al. (1995) mapped DRT, the EPHB2 gene, to 1p36.1-p35 by PCR screening of human/rodent somatic cell hybrid panels and by fluorescence in situ hybridization. As the distal end of 1p is often deleted in neuroblastomas, the DRT gene may play a role in neuroblastoma and small cell lung carcinoma (SCLC) tumorigenesis. By fluorescence in situ hybridization, Saito et al. (1995) demonstrated that the ERK gene is located in chromosomal region 1p36.1. They showed that the homologous genes are located on mouse 4D2.2-D3 and rat 5q36.13, both of which are regions with conserved linkage homology to human chromosome 1p. Animal model experiments lend further support to the function of EPHB2. Halford et al. (2000) generated mice deficient in Ryk (OMIM Ref. No. 600524) and found that they had a distinctive craniofacial appearance, shortened limbs, and postnatal mortality due to feeding and respiratory complications associated with a complete cleft of the secondary palate. Consistent with cleft palate phenocopy in Ephb2/Ephb3 (OMIM Ref. No. 601839)-deficient mice and the role of a Drosophila Ryk ortholog, ‘Derailed,’ in the transduction of repulsive axon pathfinding cues, biochemical data implicated Ryk in signaling mediated by Eph receptors and cell junction-associated Af6 (OMIM Ref. No. 159559). Halford et al. (2000) concluded that their findings highlighted the importance of signal crosstalk between members of different RTK subfamilies.

It is appreciated that the abovementioned animal model for EPHB2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Ikegaki, N.; Tang, X. X.; Liu, X.-G.; Biegel, J. A.; Allen, C.; Yoshioka, A.; Sulman, E. P.; Brodeur, G. M.; Pleasure, D. E.: Molecular characterization and chromosomal localization of DRT (EPHT3): a developmentally regulated human protein-tyrosine kinase gene of the EPH family. Hum. Molec. Genet. 4: 2033-2045, 1995.; and
Halford, M. M.; Armes, J.; Buchert, M.; Meskenaite, V.; Grail, D.; Hibbs, M. L.; Wilks, A. F.; Farlie, P. G.; Newgreen, D. F.; Hovens, C. M.; Stacker, S. A.: Ryk-deficient mice exhibit.

Further studies establishing the function and utilities of EPHB2 are found in John Hopkins OMIM database record ID 600997, and in sited publications numbered 3054, 3052-1773, 1716, 1774, 1792, 1793-1794, 177 and 1795 listed in the bibliography section hereinbelow, which are also hereby incorporated by reference. Frizzled Homolog 3 (Drosophila) (FZD3, Accession NM_{—}017412) is another VGAM227 host target gene. FZD3 BINDING SITE is HOST TARGET binding site found in the 3′ untranslated region of mRNA encoded by FZD3, corresponding to a HOST TARGET binding site such as BINDING SITE I, BINDING SITE II or BINDING SITE III. Table 2 illustrates the complementarity of the nucleotide sequences of FZD3 BINDING SITE, designated SEQ ID:1717, to the nucleotide sequence of VGAM227 RNA, herein designated VGAM RNA, also designated SEQ ID:562.

Another function of VGAM227 is therefore inhibition of Frizzled Homolog 3 (Drosophila) (FZD3, Accession NM_{—}017412), a gene which may play a role in neurogenesis during embryogenesis. Accordingly, utilities of VGAM227 include diagnosis, prevention and treatment of diseases and clinical conditions associated with FZD3. The function of FZD3 has been established by previous studies. Drosophila cuticle hairs are arranged in a defined polarity that is genetically controlled by ‘frizzled,’ a 7-transmembrane receptor with a large extracellular N-terminal cysteine-rich domain (CRD). Members of the FZD family are receptors for secreted WNT glycoproteins (see OMIM Ref. No. 602863), which are involved in developmental control. FZD proteins transmit signals through the beta-catenin (CTNNB1; 116806) or JNK (e.g., JNK3; 602897) pathways. The selection of intracellular signaling cascade may be determined by different C-terminal motifs in FZD proteins. Sala et al. (2000) determined that the FZD3 gene maps distal to the NFL gene (OMIM Ref. No. 162280) on 8p21. They mapped the mouse gene to chromosome 14, between the Gja3 and Nf1 genes in a region showing homology of synteny to human 8p21. Sala et al. (2000) noted that the human FZD3 gene is in close proximity to a putative schizophrenia susceptibility locus (SCZD6; 603013) and that WNT signaling is abnormal in some schizophrenic patients. By FISH, Kirikoshi et al. (2000) mapped the FZD3 gene to 8p21, a region associated with loss of heterozygosity in human tumors.

Full details of the abovementioned studies are described in the following publications, the disclosure of which are hereby incorporated by reference:
Kirikoshi, H.; Koike, J.; Sagara, N.; Saitoh, T.; Tokuhara, M.; Tanaka, K.; Sekihara, H.; Hirai, M.; Katoh, M.: Molecular cloning and genomic structure of human Frizzled-3 at chromosome 8p21. Biochem. Biophys. Res. Commun. 271: 8-14, 2000.; and
Sala, C. F.; Formenti, E.; Terstappen, G. C.; Caricasole, A.: Identification, gene structure, and expression of human frizzled-3 (FZD3). Biochem. Biophys. Res. Commun. 273: 27-34, 200.

Further studies establishing the function and utilities of FZD3 are found in John Hopkins OMIM database record ID 606143, and in sited publications numbered 1503-1504 listed in the bibliography section hereinbelow, which are also hereby incorporated by reference. High Mobility Group AT-hook 2 (HMGA2, Accession NM_{—}003483) is another VGAM227 host target gene. HMGA2 BINDING SITE is HOST TARGET binding site found in the 3′ untranslated region of mRNA encoded by HMGA2, corresponding to a HOST TARGET binding site such as BINDING SITE I, BINDING SITE II or BINDING SITE III. Table 2 illustrates the complementarity of the nucleotide sequences of HMGA2 BINDING SITE, designated SEQ ID: 1033, to the nucleotide sequence of VGAM227 RNA, herein designated VGAM RNA, also designated SEQ ID:562.

Another function of VGAM227 is therefore inhibition of High Mobility Group AT-hook 2 (HMGA2, Accession NM_{—}003483), a gene which may affect transcription and cell differentiation; shares common DNA-binding motif with other HMG HMG I/Y family members. Accordingly, utilities of VGAM227 include diagnosis, prevention and treatment of diseases and clinical conditions associated with HMGA2. The function of HMGA2 and its association with various diseases and clinical conditions, has been established by previous studies, as described hereinabove with reference to VGAM137. Solute Carrier Family 2 (facilitated glucose transporter), Member 4 (SLC2A4, Accession NM_{—}001042) is another VGAM227 host target gene. SLC2A4 BINDING SITE is HOST TARGET binding site found in the 3′ untranslated region of mRNA encoded by SLC2A4, corresponding to a HOST TARGET binding site such as BINDING SITE I, BINDING SITE II or BINDING SITE III. Table 2 illustrates the complementarity of the nucleotide sequences of SLC2A4 BINDING SITE, designated SEQ ID:794, to the nucleotide sequence of VGAM227 RNA, herein designated VGAM RNA, also designated SEQ ID:562.

Another function of VGAM227 is therefore inhibition of Solute Carrier Family 2 (facilitated glucose transporter), Member 4 (SLC2A4, Accession NM_{—}001042), a gene which insulin-regulated facilitative glucose transporter. Accordingly, utilities of VGAM227 include diagnosis, prevention and treatment of diseases and clinical conditions associated with SLC2A4. The function of SLC2A4 has been established by previous studies. The stimulation of glucose uptake by insulin in muscle and adipose tissue requires translocation of the GLUT4 glucose transporter from intracellular storage sites to the cell surface. Activation of phosphatidylinositol-3-OH kinase (PI3K) is required for this trafficking event, but it is not sufficient to produce GLUT4 translocation. Ribon et al. (1998) and Baumann et al. (2000) described a pathway involving the insulin-stimulated tyrosine phosphorylation of CBL (OMIM Ref. No. 165360), which is recruited to the insulin receptor (OMIM Ref. No. 147670) by the adaptor protein CAP (OMIM Ref. No. 605264). On phosphorylation, CBL is translocated to lipid rafts. Blocking this step completely inhibits the stimulation of GLUT4 translocation by insulin. Chiang et al. (2001) showed that phosphorylated CBL recruits the CRK2-C3G (164762, 600303) complex to lipid rafts, where C3G specifically activates the small GTP-binding protein TC10 (OMIM Ref. No. 605857). This process is independent of PI3K, but requires the translocation of CBL, CRK, and C3G to the lipid raft. The activation of TC10 is essential for insulin-stimulated glucose uptake and GLUT4 translocation. The TC10 pathway functions in parallel with PI3K to stimulate fully GLUT4 translocation in response to insulin. Animal model experiments lend further support to the function of SLC2A4. To determine the role of adipose GLUT4 in glucose homeostasis, Abel et al. (2001) used Cre/loxP DNA recombination to generate mice with adipose-selective reduction of GLUT4 (G4A −/−). G4A −/− mice had normal growth and adipose mass despite markedly impaired insulin-stimulated glucose uptake in adipocytes. Although GLUT4 expression is preserved in muscle, these mice developed insulin resistance in muscle and liver, manifested by decreased biologic responses and impaired activation of phosphoinositide-3-OH kinase (see OMIM Ref. No. 601232). G4A −/− mice developed glucose intolerance and hyperinsulinemia. Thus, downregulation of GLUT4 and glucose transport selectively in adipose tissue can cause insulin resistance and thereby increase the risk of developing diabetes. In G4A −/− mice, mean plasma leptin levels were normal and plasma leptin concentrations showed the same linear relationship with body weight in G4A −/− mice as in control littermates. Thus, normal glucose uptake in adipocytes is not necessary to maintain normal plasma leptin levels. Elevated TNF-alpha (OMIM Ref. No. 191160) was noted in G4A −/− mice.

It is appreciated that the abovementioned animal model for SLC2A4 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Abel, E. D.; Peroni, O.; Kim, J. K.; Kim, Y.-B.; Boss, O.; Hadro, E.; Minnemann, T.; Shulman, G. I.; Kahn, B. B.: Adipose-selective targeting of the GLUT4 gene impairs insulin action in muscle and liver. Nature 409: 729-733, 2001.; and
Chiang, S.-H.; Baumann, C. A.; Kanzaki, M.; Thurmond, D. C.; Watson, R. T.; Neudauer, C. L.; Macara, I. G.; Pessin, J. E.; Saltiel, A. R.: Insulin-stimulated GLUT4 translocation requires.

Further studies establishing the function and utilities of SLC2A4 are found in John Hopkins OMIM database record ID 138190, and in sited publications numbered 2775-2776, 2633, 2777-2780, 275 and 2781-2787 listed in the bibliography section hereinbelow, which are also hereby incorporated by reference. ATPase, Class V, Type 10B (ATP10B, Accession XM_{—}032721) is another VGAM227 host target gene. ATP10B BINDING SITE is HOST TARGET binding site found in the 3′ untranslated region of mRNA encoded by ATP10B, corresponding to a HOST TARGET binding site such as BINDING SITE I, BINDING SITE II or BINDING SITE III. Table 2 illustrates the complementarity of the nucleotide sequences of ATP10B BINDING SITE, designated SEQ ID:2654, to the nucleotide sequence of VGAM227 RNA, herein designated VGAM RNA, also designated SEQ ID:562.

Another function of VGAM227 is therefore inhibition of ATPase, Class V, Type 10B (ATP10B, Accession XM_{—}032721). Accordingly, utilities of VGAM227 include diagnosis, prevention and treatment of diseases and clinical conditions associated with ATP10B. BRCA2 and CDKN1A Interacting Protein (BCCIP, Accession NM_{—}078469) is another VGAM227 host target gene. BCCIP BINDING SITE is HOST TARGET binding site found in the 3′ untranslated region of mRNA encoded by BCCIP, corresponding to a HOST TARGET binding site such as BINDING SITE I, BINDING SITE II or BINDING SITE III. Table 2 illustrates the complementarity of the nucleotide sequences of BCCIP BINDING SITE, designated SEQ ID:2369, to the nucleotide sequence of VGAM227 RNA, herein designated VGAM RNA, also designated SEQ ID:562.

Another function of VGAM227 is therefore inhibition of BRCA2 and CDKN1A Interacting Protein (BCCIP, Accession NM_{—}078469). Accordingly, utilities of VGAM227 include diagnosis, prevention and treatment of diseases and clinical conditions associated with BCCIP. Chromosome 20 Open Reading Frame 30 (C20orf30, Accession NM_{—}014145) is another VGAM227 host target gene. C20orf30 BINDING SITE is HOST TARGET binding site found in the 3′ untranslated region of mRNA encoded by C20orf30, corresponding to a HOST TARGET binding site such as BINDING SITE I, BINDING SITE II or BINDING SITE III. Table 2 illustrates the complementarity of the nucleotide sequences of C20orf30 BINDING SITE, designated SEQ ID:1478, to the nucleotide sequence of VGAM227 RNA, herein designated VGAM RNA, also designated SEQ ID:562.

Another function of VGAM227 is therefore inhibition of Chromosome 20 Open Reading Frame 30 (C20orf30, Accession NM_{—}014145). Accordingly, utilities of VGAM227 include diagnosis, prevention and treatment of diseases and clinical conditions associated with C20orf30. DKFZP5640043 (Accession XM_{—}166502) is another VGAM227 host target gene. DKFZP5640043 BINDING SITE is HOST TARGET binding site found in the 3′ untranslated region of mRNA encoded by DKFZP5640043, corresponding to a HOST TARGET binding site such as BINDING SITE I, BINDING SITE II or BINDING SITE III. Table 2 illustrates the complementarity of the nucleotide sequences of DKFZP5640043 BINDING SITE, designated SEQ ID:3565, to the nucleotide sequence of VGAM227 RNA, herein designated VGAM RNA, also designated SEQ ID:562.

Another function of VGAM227 is therefore inhibition of DKFZP5640043 (Accession XM_{—}166502). Accordingly, utilities of VGAM227 include diagnosis, prevention and treatment of diseases and clinical conditions associated with DKFZP5640043. FLJ11710 (Accession NM_{—}024846) is another VGAM227 host target gene. FLJ11710 BINDING SITE is HOST TARGET binding site found in the 3′ untranslated region of mRNA encoded by FLJ11710, corresponding to a HOST TARGET binding site such as BINDING SITE I, BINDING SITE II or BINDING SITE III. Table 2 illustrates the complementarity of the nucleotide sequences of FLJ11710 BINDING SITE, designated SEQ ID:2097, to the nucleotide sequence of VGAM227 RNA, herein designated VGAM RNA, also designated SEQ ID:562.

Another function of VGAM227 is therefore inhibition of FLJ11710 (Accession NM_{—}024846). Accordingly, utilities of VGAM227 include diagnosis, prevention and treatment of diseases and clinical conditions associated with FLJ11710. JDD1 (Accession XM_{—}032515) is another VGAM227 host target gene. JDD1 BINDING SITE is HOST TARGET binding site found in the 3′ untranslated region of mRNA encoded by JDD1, corresponding to a HOST TARGET binding site such as BINDING SITE I, BINDING SITE II or BINDING SITE III. Table 2 illustrates the complementarity of the nucleotide sequences of JDD1 BINDING SITE, designated SEQ ID:2653, to the nucleotide sequence of VGAM227 RNA, herein designated VGAM RNA, also designated SEQ ID:562.

Another function of VGAM227 is therefore inhibition of JDD1 (Accession XM_{—}032515). Accordingly, utilities of VGAM227 include diagnosis, prevention and treatment of diseases and clinical conditions associated with JDD1. KIAA0265 (Accession XM_{—}045954) is another VGAM227 host target gene. KIAA0265 BINDING SITE is HOST TARGET binding site found in the 3′ untranslated region of mRNA encoded by KIAA0265, corresponding to a HOST TARGET binding site such as BINDING SITE I, BINDING SITE II or BINDING SITE III. Table 2 illustrates the complementarity of the nucleotide sequences of KIAA0265 BINDING SITE, designated SEQ ID:2871, to the nucleotide sequence of VGAM227 RNA, herein designated VGAM RNA, also designated SEQ ID:562.

Another function of VGAM227 is therefore inhibition of KIAA0265 (Accession XM_{—}045954). Accordingly, utilities of VGAM227 include diagnosis, prevention and treatment of diseases and clinical conditions associated with KIAA0265. KIAA0663 (Accession NM_{—}014827) is another VGAM227 host target gene. KIAA0663 BINDING SITE is HOST TARGET binding site found in the 3′ untranslated region of mRNA encoded by KIAA0663, corresponding to a HOST TARGET binding site such as BINDING SITE I, BINDING SITE II or BINDING SITE III. Table 2 illustrates the complementarity of the nucleotide sequences of KIAA0663 BINDING SITE, designated SEQ ID:1561, to the nucleotide sequence of VGAM227 RNA, herein designated VGAM RNA, also designated SEQ ID:562.

Another function of VGAM227 is therefore inhibition of KIAA0663 (Accession NM_{—}014827). Accordingly, utilities of VGAM227 include diagnosis, prevention and treatment of diseases and clinical conditions associated with KIAA0663. KIAA1010 (Accession XM_{—}050742) is another VGAM227 host target gene. KIAA1010 BINDING SITE is HOST TARGET binding site found in the 3′ untranslated region of mRNA encoded by KIAA1010, corresponding to a HOST TARGET binding site such as BINDING SITE I, BINDING SITE II or BINDING SITE III. Table 2 illustrates the complementarity of the nucleotide sequences of KIAA1010 BINDING SITE, designated SEQ ID:2934, to the nucleotide sequence of VGAM227 RNA, herein designated VGAM RNA, also designated SEQ ID:562.

Another function of VGAM227 is therefore inhibition of KIAA1010 (Accession XM_{—}050742). Accordingly, utilities of VGAM227 include diagnosis, prevention and treatment of diseases and clinical conditions associated with KIAA1010. KIAA1576 (Accession XM_{—}038186) is another VGAM227 host target gene. KIAA1576 BINDING SITE is HOST TARGET binding site found in the 3′ untranslated region of mRNA encoded by KIAA1576, corresponding to a HOST TARGET binding site such as BINDING SITE I, BINDING SITE II or BINDING SITE III. Table 2 illustrates the complementarity of the nucleotide sequences of KIAA1576 BINDING SITE, designated SEQ ID:2734, to the nucleotide sequence of VGAM227 RNA, herein designated VGAM RNA, also designated SEQ ID:562.

Another function of VGAM227 is therefore inhibition of KIAA1576 (Accession XM_{—}038186). Accordingly, utilities of VGAM227 include diagnosis, prevention and treatment of diseases and clinical conditions associated with KIAA1576. KIAA1634 (Accession XM_{—}032749) is another VGAM227 host target gene. KIAA1634 BINDING SITE is HOST TARGET binding site found in the 3′ untranslated region of mRNA encoded by KIAA1634, corresponding to a HOST TARGET binding site such as BINDING SITE I, BINDING SITE II or BINDING SITE III. Table 2 illustrates the complementarity of the nucleotide sequences of KIAA1634 BINDING SITE, designated SEQ ID:2658, to the nucleotide sequence of VGAM227 RNA, herein designated VGAM RNA, also designated SEQ ID:562.

Another function of VGAM227 is therefore inhibition of KIAA1634 (Accession XM_{—}032749). Accordingly, utilities of VGAM227 include diagnosis, prevention and treatment of diseases and clinical conditions associated with KIAA1634. KIAA1798 (Accession XM_{—}027074) is another VGAM227 host target gene. KIAA1798 BINDING SITE is HOST TARGET binding site found in the 3′ untranslated region of mRNA encoded by KIAA1798, corresponding to a HOST TARGET binding site such as BINDING SITE I, BINDING SITE II or BINDING SITE III. Table 2 illustrates the complementarity of the nucleotide sequences of KIAA1798 BINDING SITE, designated SEQ ID:2575, to the nucleotide sequence of VGAM227 RNA, herein designated VGAM RNA, also designated SEQ ID:562.

Another function of VGAM227 is therefore inhibition of KIAA1798 (Accession XM_{—}027074). Accordingly, utilities of VGAM227 include diagnosis, prevention and treatment of diseases and clinical conditions associated with KIAA1798. Kinesin-like 7 (KNSL7, Accession NM_{—}020242) is another VGAM227 host target gene. KNSL7 BINDING SITE is HOST TARGET binding site found in the 3′ untranslated region of mRNA encoded by KNSL7, corresponding to a HOST TARGET binding site such as BINDING SITE I, BINDING SITE II or BINDING SITE III. Table 2 illustrates the complementarity of the nucleotide sequences of KNSL7 BINDING SITE, designated SEQ ID:1904, to the nucleotide sequence of VGAM227 RNA, herein designated VGAM RNA, also designated SEQ ID:562.

Another function of VGAM227 is therefore inhibition of Kinesin-like 7 (KNSL7, Accession NM_{—}020242). Accordingly, utilities of VGAM227 include diagnosis, prevention and treatment of diseases and clinical conditions associated with KNSL7. LALP1 (Accession NM_{—}020354) is another VGAM227 host target gene. LALP1 BINDING SITE is HOST TARGET binding site found in the 3′ untranslated region of mRNA encoded by LALP1, corresponding to a HOST TARGET binding site such as BINDING SITE I, BINDING SITE II or BINDING SITE III. Table 2 illustrates the complementarity of the nucleotide sequences of LALP1 BINDING SITE, designated SEQ ID:1916, to the nucleotide sequence of VGAM227 RNA, herein designated VGAM RNA, also designated SEQ ID:562.

Another function of VGAM227 is therefore inhibition of LALP1 (Accession NM_{—}020354). Accordingly, utilities of VGAM227 include diagnosis, prevention and treatment of diseases and clinical conditions associated with LALP1. MGC2488 (Accession NM_{—}024039) is another VGAM227 host target gene. MGC2488 BINDING SITE is HOST TARGET binding site found in the 3′ untranslated region of mRNA encoded by MGC2488, corresponding to a HOST TARGET binding site such as BINDING SITE I, BINDING SITE II or BINDING SITE III. Table 2 illustrates the complementarity of the nucleotide sequences of MGC2488 BINDING SITE, designated SEQ ID:2044, to the nucleotide sequence of VGAM227 RNA, herein designated VGAM RNA, also designated SEQ ID:562.

Another function of VGAM227 is therefore inhibition of MGC2488 (Accession NM_{—}024039). Accordingly, utilities of VGAM227 include diagnosis, prevention and treatment of diseases and clinical conditions associated with MGC2488. nexilin (Accession NM_{—}144573) is another VGAM227 host target gene. nexilin BINDING SITE is HOST TARGET binding site found in the 3′ untranslated region of mRNA encoded by nexilin, corresponding to a HOST TARGET binding site such as BINDING SITE I, BINDING SITE II or BINDING SITE III. Table 2 illustrates the complementarity of the nucleotide sequences of nexilin BINDING SITE, designated SEQ ID:2484, to the nucleotide sequence of VGAM227 RNA, herein designated VGAM RNA, also designated SEQ ID:562.

Another function of VGAM227 is therefore inhibition of nexilin (Accession NM_{—}144573). Accordingly, utilities of VGAM227 include diagnosis, prevention and treatment of diseases and clinical conditions associated with nexilin. NKX2B (Accession NM_{—}002509) is another VGAM227 host target gene. NKX2B BINDING SITE is HOST TARGET binding site found in the 3′ untranslated region of mRNA encoded by NKX2B, corresponding to a HOST TARGET binding site such as BINDING SITE I, BINDING SITE II or BINDING SITE III. Table 2 illustrates the complementarity of the nucleotide sequences of NKX2B BINDING SITE, designated SEQ ID:934, to the nucleotide sequence of VGAM227 RNA, herein designated VGAM RNA, also designated SEQ ID:562.

Another function of VGAM227 is therefore inhibition of NKX2B (Accession NM_{—}002509). Accordingly, utilities of VGAM227 include diagnosis, prevention and treatment of diseases and clinical conditions associated with NKX2B. Syntaxin 18 (STX18, Accession NM_{—}016930) is another VGAM227 host target gene. STX18 BINDING SITE is HOST TARGET binding site found in the 3′ untranslated region of mRNA encoded by STX18, corresponding to a HOST TARGET binding site such as BINDING SITE I, BINDING SITE II or BINDING SITE III. Table 2 illustrates the complementarity of the nucleotide sequences of STX18 BINDING SITE, designated SEQ ID:1715, to the nucleotide sequence of VGAM227 RNA, herein designated VGAM RNA, also designated SEQ ID:562.

Another function of VGAM227 is therefore inhibition of Syntaxin 18 (STX18, Accession NM_{—}016930). Accordingly, utilities of VGAM227 include diagnosis, prevention and treatment of diseases and clinical conditions associated with STX18. Zinc Finger Protein 271 (ZNF271, Accession XM_{—}170865) is another VGAM227 host target gene. ZNF271 BINDING SITE is HOST TARGET binding site found in the 5′ untranslated region of mRNA encoded by ZNF271, corresponding to a HOST TARGET binding site such as BINDING SITE I, BINDING SITE II or BINDING SITE III. Table 2 illustrates the complementarity of the nucleotide sequences of ZNF271 BINDING SITE, designated SEQ ID:3670, to the nucleotide sequence of VGAM227 RNA, herein designated VGAM RNA, also designated SEQ ID:562.

Another function of VGAM227 is therefore inhibition of Zinc Finger Protein 271 (ZNF271, Accession XM_{—}170865). Accordingly, utilities of VGAM227 include diagnosis, prevention and treatment of diseases and clinical conditions associated with ZNF271. LOC115207 (Accession NM_{—}138444) is another VGAM227 host target gene. LOC115207 BINDING SITE is HOST TARGET binding site found in the 3′ untranslated region of mRNA encoded by LOC115207, corresponding to a HOST TARGET binding site such as BINDING SITE I, BINDING SITE II or BINDING SITE III. Table 2 illustrates the complementarity of the nucleotide sequences of LOC115207 BINDING SITE, designated SEQ ID:2440, to the nucleotide sequence of VGAM227 RNA, herein designated VGAM RNA, also designated SEQ ID:562.

Another function of VGAM227 is therefore inhibition of LOC115207 (Accession NM_{—}138444). Accordingly, utilities of VGAM227 include diagnosis, prevention and treatment of diseases and clinical conditions associated with LOC115207. LOC133482 (Accession XM_{—}059654) is another VGAM227 host target gene. LOC133482 BINDING SITE is HOST TARGET binding site found in the 5′ untranslated region of mRNA encoded by LOC133482, corresponding to a HOST TARGET binding site such as BINDING SITE I, BINDING SITE II or BINDING SITE III. Table 2 illustrates the complementarity of the nucleotide sequences of LOC133482 BINDING SITE, designated SEQ ID:3014, to the nucleotide sequence of VGAM227 RNA, herein designated VGAM RNA, also designated SEQ ID:562.

Another function of VGAM227 is therefore inhibition of LOC133482 (Accession XM_{—}059654). Accordingly, utilities of VGAM227 include diagnosis, prevention and treatment of diseases and clinical conditions associated with LOC133482. LOC147080 (Accession XM_{—}097182) is another VGAM227 host target gene. LOC147080 BINDING SITE is HOST TARGET binding site found in the 5′ untranslated region of mRNA encoded by LOC147080, corresponding to a HOST TARGET binding site such as BINDING SITE I, BINDING SITE II or BINDING SITE III. Table 2 illustrates the complementarity of the nucleotide sequences of LOC147080 BINDING SITE, designated SEQ ID:3290, to the nucleotide sequence of VGAM227 RNA, herein designated VGAM RNA, also designated SEQ ID:562.

Another function of VGAM227 is therefore inhibition of LOC147080 (Accession XM_{—}097182). Accordingly, utilities of VGAM227 include diagnosis, prevention and treatment of diseases and clinical conditions associated with LOC147080. LOC147990 (Accession XM_{—}097358) is another VGAM227 host target gene. LOC147990 BINDING SITE is HOST TARGET binding site found in the 3′ untranslated region of mRNA encoded by LOC147990, corresponding to a HOST TARGET binding site such as BINDING SITE I, BINDING SITE II or BINDING SITE III. Table 2 illustrates the complementarity of the nucleotide sequences of LOC147990 BINDING SITE, designated SEQ ID:3295, to the nucleotide sequence of VGAM227 RNA, herein designated VGAM RNA, also designated SEQ ID:562.

Another function of VGAM227 is therefore inhibition of LOC147990 (Accession XM_{—}097358). Accordingly, utilities of VGAM227 include diagnosis, prevention and treatment of diseases and clinical conditions associated with LOC147990. LOC149529 (Accession XM_{—}086562) is another VGAM227 host target gene. LOC149529 BINDING SITE is HOST TARGET binding site found in the 3′ untranslated region of mRNA encoded by LOC149529, corresponding to a HOST TARGET binding site such as BINDING SITE I, BINDING SITE II or BINDING SITE III. Table 2 illustrates the complementarity of the nucleotide sequences of LOC149529 BINDING SITE, designated SEQ ID:3134, to the nucleotide sequence of VGAM227 RNA, herein designated VGAM RNA, also designated SEQ ID:562.

Another function of VGAM227 is therefore inhibition of LOC149529 (Accession XM_{—}086562). Accordingly, utilities of VGAM227 include diagnosis, prevention and treatment of diseases and clinical conditions associated with LOC149529. LOC158130 (Accession XM_{—}044880) is another VGAM227 host target gene. LOC158130 BINDING SITE is HOST TARGET binding site found in the 3′ untranslated region of mRNA encoded by LOC158130, corresponding to a HOST TARGET binding site such as BINDING SITE I, BINDING SITE II or BINDING SITE III. Table 2 illustrates the complementarity of the nucleotide sequences of LOC158130 BINDING SITE, designated SEQ ID:2847, to the nucleotide sequence of VGAM227 RNA, herein designated VGAM RNA, also designated SEQ ID:562.

Another function of VGAM227 is therefore inhibition of LOC158130 (Accession XM_{—}044880). Accordingly, utilities of VGAM227 include diagnosis, prevention and treatment of diseases and clinical conditions associated with LOC158130. LOC196478 (Accession XM_{—}113729) is another VGAM227 host target gene. LOC196478 BINDING SITE is HOST TARGET binding site found in the 5′ untranslated region of mRNA encoded by LOC196478, corresponding to a HOST TARGET binding site such as BINDING SITE I, BINDING SITE II or BINDING SITE III. Table 2 illustrates the complementarity of the nucleotide sequences of LOC196478 BINDING SITE, designated SEQ ID:3413, to the nucleotide sequence of VGAM227 RNA, herein designated VGAM RNA, also designated SEQ ID:562.

Another function of VGAM227 is therefore inhibition of LOC196478 (Accession XM_{—}113729). Accordingly, utilities of VGAM227 include diagnosis, prevention and treatment of diseases and clinical conditions associated with LOC196478. LOC219894 (Accession XM_{—}167782) is another VGAM227 host target gene. LOC219894 BINDING SITE is HOST TARGET binding site found in the 3′ untranslated region of mRNA encoded by LOC219894, corresponding to a HOST TARGET binding site such as BINDING SITE I, BINDING SITE II or BINDING SITE III. Table 2 illustrates the complementarity of the nucleotide sequences of LOC219894 BINDING SITE, designated SEQ ID:3595, to the nucleotide sequence of VGAM227 RNA, herein designated VGAM RNA, also designated SEQ ID:562.

Another function of VGAM227 is therefore inhibition of LOC219894 (Accession XM_{—}167782). Accordingly, utilities of VGAM227 include diagnosis, prevention and treatment of diseases and clinical conditions associated with LOC219894. LOC219988 (Accession XM_{—}166223) is another VGAM227 host target gene. LOC219988 BINDING SITE is HOST TARGET binding site found in the 5′ untranslated region of mRNA encoded by LOC219988, corresponding to a HOST TARGET binding site such as BINDING SITE I, BINDING SITE II or BINDING SITE III. Table 2 illustrates the complementarity of the nucleotide sequences of LOC219988 BINDING SITE, designated SEQ ID:3535, to the nucleotide sequence of VGAM227 RNA, herein designated VGAM RNA, also designated SEQ ID:562.

Another function of VGAM227 is therefore inhibition of LOC219988 (Accession XM_{—}166223). Accordingly, utilities of VGAM227 include diagnosis, prevention and treatment of diseases and clinical conditions associated with LOC219988. LOC221272 (Accession XM_{—}168050) is another VGAM227 host target gene. LOC221272 BINDING SITE1 and LOC221272 BINDING SITE2 are HOST TARGET binding sites found in untranslated regions of mRNA encoded by LOC221272, corresponding to HOST TARGET binding sites such as BINDING SITE I, BINDING SITE II or BINDING SITE III. Table 2 illustrates the complementarity of the nucleotide sequences of LOC221272 BINDING SITE1 and LOC221272 BINDING SITE2, designated SEQ ID:3613 and SEQ ID:3614 respectively, to the nucleotide sequence of VGAM227 RNA, herein designated VGAM RNA, also designated SEQ ID:562.

Another function of VGAM227 is therefore inhibition of LOC221272 (Accession XM_{—}168050). Accordingly, utilities of VGAM227 include diagnosis, prevention and treatment of diseases and clinical conditions associated with LOC221272. LOC253019 (Accession XM_{—}170907) is another VGAM227 host target gene. LOC253019 BINDING SITE is HOST TARGET binding site found in the 3′ untranslated region of mRNA encoded by LOC253019, corresponding to a HOST TARGET binding site such as BINDING SITE I, BINDING SITE II or BINDING SITE III. Table 2 illustrates the complementarity of the nucleotide sequences of LOC253019 BINDING SITE, designated SEQ ID:3673, to the nucleotide sequence of VGAM227 RNA, herein designated VGAM RNA, also designated SEQ ID:562.

Another function of VGAM227 is therefore inhibition of LOC253019 (Accession XM_{—}170907). Accordingly, utilities of VGAM227 include diagnosis, prevention and treatment of diseases and clinical conditions associated with LOC253019. LOC253975 (Accession XM_{—}171130) is another VGAM227 host target gene. LOC253975 BINDING SITE is HOST TARGET binding site found in the 3′ untranslated region of mRNA encoded by LOC253975, corresponding to a HOST TARGET binding site such as BINDING SITE I, BINDING SITE II or BINDING SITE III. Table 2 illustrates the complementarity of the nucleotide sequences of LOC253975 BINDING SITE, designated SEQ ID:3687, to the nucleotide sequence of VGAM227 RNA, herein designated VGAM RNA, also designated SEQ ID:562.

Another function of VGAM227 is therefore inhibition of LOC253975 (Accession XM_{—}171130). Accordingly, utilities of VGAM227 include diagnosis, prevention and treatment of diseases and clinical conditions associated with LOC253975. LOC254873 (Accession XM_{—}170557) is another VGAM227 host target gene. LOC254873 BINDING SITE is HOST TARGET binding site found in the 3′ untranslated region of mRNA encoded by LOC254873, corresponding to a HOST TARGET binding site such as BINDING SITE I, BINDING SITE II or BINDING SITE III. Table 2 illustrates the complementarity of the nucleotide sequences of LOC254873 BINDING SITE, designated SEQ ID:3659, to the nucleotide sequence of VGAM227 RNA, herein designated VGAM RNA, also designated SEQ ID:562.

Another function of VGAM227 is therefore inhibition of LOC254873 (Accession XM_{—}170557). Accordingly, utilities of VGAM227 include diagnosis, prevention and treatment of diseases and clinical conditions associated with LOC254873. LOC51634 (Accession NM_{—}016024) is another VGAM227 host target gene. LOC51634 BINDING SITE is HOST TARGET binding site found in the 3′ untranslated region of mRNA encoded by LOC51634, corresponding to a HOST TARGET binding site such as BINDING SITE I, BINDING SITE II or BINDING SITE III. Table 2 illustrates the complementarity of the nucleotide sequences of LOC51634 BINDING SITE, designated SEQ ID:1655, to the nucleotide sequence of VGAM227 RNA, herein designated VGAM RNA, also designated SEQ ID:562.

Another function of VGAM227 is therefore inhibition of LOC51634 (Accession NM_{—}016024). Accordingly, utilities of VGAM227 include diagnosis, prevention and treatment of diseases and clinical conditions associated with LOC51634. FIG. 1 further provides a conceptual description of a novel bioinformatically detected viral gene of the present invention, referred to here as Viral Genomic Address Messenger 228 (VGAM228) viral gene, which modulates expression of respective host target genes thereof, the function and utility of which host target genes is known in the art.

VGAM228 is a novel bioinformatically detected regulatory, non protein coding, viral micro RNA (miRNA) gene. The method by which VGAM228 was detected is described hereinabove with reference to FIGS. 1-8.

VGAM228 gene, herein designated VGAM GENE, is a viral gene contained in the genome of Vaccinia Virus. VGAM228 host target gene, herein designated VGAM HOST TARGET GENE, is a human gene contained in the human genome.

VGAM228 gene encodes a VGAM228 precursor RNA, herein designated VGAM PRECURSOR RNA. Similar to other miRNA genes, and unlike most ordinary genes, VGAM228 precursor RNA does not encode a protein. A nucleotide sequence identical or highly similar to the nucleotide sequence of VGAM228 precursor RNA is designated SEQ ID:214, and is provided hereinbelow with reference to the sequence listing part. Nucleotide sequence SEQ ID:214 is located at position 120881 relative to the genome of Vaccinia Virus.

VGAM228 precursor RNA folds onto itself, forming VGAM228 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM228 folded precursor RNA into VGAM228 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM228 RNA is designated SEQ ID:563, and is provided hereinbelow with reference to the sequence listing part.

VGAM228 host target gene, herein designated VGAM HOST TARGET GENE, encodes a corresponding messenger RNA, VGAM228 host target RNA, herein designated VGAM HOST TARGET RNA. VGAM228 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM228 RNA, herein designated VGAM RNA, binds complementarily to one or more host target binding sites located in untranslated regions of VGAM228 host target RNA, herein designated VGAM HOST TARGET RNA. This complementary binding is due to the fact that the nucleotide sequence of VGAM228 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM228 RNA, herein designated VGAM RNA, may have a different number of host target binding sites in untranslated regions of a VGAM228 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM228 RNA, herein designated VGAM RNA, to host target binding sites on VGAM228 host target RNA, herein designated VGAM HOST TARGET RNA, such as BINDING SITE I, BINDING SITE II and BINDING SITE III, inhibits translation of VGAM228 host target RNA into VGAM228 host target protein, herein designated VGAM HOST TARGET PROTEIN. VGAM host target protein is therefore outlined by a broken line.

It is appreciated that VGAM228 host target gene, herein designated VGAM HOST TARGET GENE, in fact represents a plurality of VGAM228 host target genes. The mRNA of each one of this plurality of VGAM228 host target genes comprises one or more host target binding sites, each having a nucleotide sequence which is at least partly complementary to VGAM228 RNA, herein designated VGAM RNA, and which when bound by VGAM228 RNA causes inhibition of translation of respective one or more VGAM228 host target proteins.

It is further appreciated by one skilled in the art that the mode of translational inhibition illustrated by FIG. 1 with specific reference to translational inhibition exerted by VGAM228 gene, herein designated VGAM GENE, on one or more VGAM228 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM228 is inhibition of expression of host target genes, as part of a novel viral mechanism of attacking a host. Accordingly, utilities of VGAM228 include diagnosis, prevention and treatment of viral infection by Vaccinia Virus. Specific functions, and accordingly utilities, of VGAM228 correlate with, and may be deduced from, the identity of the host target genes which VGAM228 binds and inhibits, and the function of these host target genes, as elaborated hereinbelow.

Nucleotide sequences of the VGAM228 precursor RNA, herein designated VGAM PRECURSOR RNA, and of the ‘diced’ VGAM228 RNA, herein designated VGAM RNA, and a schematic representation of the secondary folding of VGAM228 folded precursor RNA, herein designated VGAM FOLDED PRECURSOR RNA, of VGAM228 are further described hereinbelow with reference to Table 1.

Nucleotide sequences of host target binding sites, such as BINDING SITE-I, BINDING SITE-II and BINDING SITE-III of FIG. 1, found on VGAM228 host target RNA, and schematic representation of the complementarity of each of these host target binding sites to VGAM228 RNA, herein designated VGAM RNA, are described hereinbelow with reference to Table 2.

As mentioned hereinabove with reference to FIG. 1, a function of VGAM228 gene, herein designated VGAM is inhibition of expression of VGAM228 target genes. It is appreciated that specific functions, and accordingly utilities, of VGAM228 correlate with, and may be deduced from, the identity of the target genes which VGAM228 binds and inhibits, and the function of these target genes, as elaborated hereinbelow.

ATP-binding Cassette, Sub-family E (OABP), Member 1 (ABCE1, Accession XM_{—}003555) is a VGAM228 host target gene. ABCE1 BINDING SITE is HOST TARGET binding site found in the 3′ untranslated region of mRNA encoded by ABCE1, corresponding to a HOST TARGET binding site such as BINDING SITE I, BINDING SITE II or BINDING SITE III. Table 2 illustrates the complementarity of the nucleotide sequences of ABCE1 BINDING SITE, designated SEQ ID:2532, to the nucleotide sequence of VGAM228 RNA, herein designated VGAM RNA, also designated SEQ ID:563.

A function of VGAM228 is therefore inhibition of ATP-binding Cassette, Sub-family E (OABP), Member 1 (ABCE1, Accession XM_{—}003555), a gene which a Ribonuclease L inhibitor. Accordingly, utilities of VGAM228 include diagnosis, prevention and treatment of diseases and clinical conditions associated with ABCE1. The function of ABCE1 and its association with various diseases and clinical conditions, has been established by previous studies, as described hereinabove with reference to VGAM172. WWP1 (Accession NM_{—}007013) is another VGAM228 host target gene. WWP1 BINDING SITE is HOST TARGET binding site found in the 3′ untranslated region of mRNA encoded by WWP1, corresponding to a HOST TARGET binding site such as BINDING SITE I, BINDING SITE II or BINDING SITE III. Table 2 illustrates the complementarity of the nucleotide sequences of WWP1 BINDING SITE, designated SEQ ID:1345, to the nucleotide sequence of VGAM228 RNA, herein designated VGAM RNA, also designated SEQ ID:563.

Another function of VGAM228 is therefore inhibition of WWP1 (Accession NM_{—}007013). Accordingly, utilities of VGAM228 include diagnosis, prevention and treatment of diseases and clinical conditions associated with WWP1. FIG. 1 further provides a conceptual description of a novel bioinformatically detected viral gene of the present invention, referred to here as Viral Genomic Address Messenger 229 (VGAM229) viral gene, which modulates expression of respective host target genes thereof, the function and utility of which host target genes is known in the art.

VGAM229 is a novel bioinformatically detected regulatory, non protein coding, viral micro RNA (miRNA) gene. The method by which VGAM229 was detected is described hereinabove with reference to FIGS. 1-8.

VGAM229 gene, herein designated VGAM GENE, is a viral gene contained in the genome of Vaccinia Virus. VGAM229 host target gene, herein designated VGAM HOST TARGET GENE, is a human gene contained in the human genome.

VGAM229 gene encodes a VGAM229 precursor RNA, herein designated VGAM PRECURSOR RNA. Similar to other miRNA genes, and unlike most ordinary genes, VGAM229 precursor RNA does not encode a protein. A nucleotide sequence identical or highly similar to the nucleotide sequence of VGAM229 precursor RNA is designated SEQ ID:215, and is provided hereinbelow with reference to the sequence listing part. Nucleotide sequence SEQ ID:215 is located at position 122419 relative to the genome of Vaccinia Virus.

VGAM229 precursor RNA folds onto itself, forming VGAM229 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM229 folded precursor RNA into VGAM229 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM229 RNA is designated SEQ ID:564, and is provided hereinbelow with reference to the sequence listing part.

VGAM229 host target gene, herein designated VGAM HOST TARGET GENE, encodes a corresponding messenger RNA, VGAM229 host target RNA, herein designated VGAM HOST TARGET RNA. VGAM229 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM229 RNA, herein designated VGAM RNA, binds complementarily to one or more host target binding sites located in untranslated regions of VGAM229 host target RNA, herein designated VGAM HOST TARGET RNA. This complementary binding is due to the fact that the nucleotide sequence of VGAM229 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM229 RNA, herein designated VGAM RNA, may have a different number of host target binding sites in untranslated regions of a VGAM229 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM229 RNA, herein designated VGAM RNA, to host target binding sites on VGAM229 host target RNA, herein designated VGAM HOST TARGET RNA, such as BINDING SITE I, BINDING SITE II and BINDING SITE III, inhibits translation of VGAM229 host target RNA into VGAM229 host target protein, herein designated VGAM HOST TARGET PROTEIN. VGAM host target protein is therefore outlined by a broken line.

It is appreciated that VGAM229 host target gene, herein designated VGAM HOST TARGET GENE, in fact represents a plurality of VGAM229 host target genes. The mRNA of each one of this plurality of VGAM229 host target genes comprises one or more host target binding sites, each having a nucleotide sequence which is at least partly complementary to VGAM229 RNA, herein designated VGAM RNA, and which when bound by VGAM229 RNA causes inhibition of translation of respective one or more VGAM229 host target proteins.

It is further appreciated by one skilled in the art that the mode of translational inhibition illustrated by FIG. 1 with specific reference to translational inhibition exerted by VGAM229 gene, herein designated VGAM GENE, on one or more VGAM229 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM229 is inhibition of expression of host target genes, as part of a novel viral mechanism of attacking a host. Accordingly, utilities of VGAM229 include diagnosis, prevention and treatment of viral infection by Vaccinia Virus. Specific functions, and accordingly utilities, of VGAM229 correlate with, and may be deduced from, the identity of the host target genes which VGAM229 binds and inhibits, and the function of these host target genes, as elaborated hereinbelow.

Nucleotide sequences of the VGAM229 precursor RNA, herein designated VGAM PRECURSOR RNA, and of the ‘diced’ VGAM229 RNA, herein designated VGAM RNA, and a schematic representation of the secondary folding of VGAM229 folded precursor RNA, herein designated VGAM FOLDED PRECURSOR RNA, of VGAM229 are further described hereinbelow with reference to Table 1.

Nucleotide sequences of host target binding sites, such as BINDING SITE-I, BINDING SITE-II and BINDING SITE-III of FIG. 1, found on VGAM229 host target RNA, and schematic representation of the complementarity of each of these host target binding sites to VGAM229 RNA, herein designated VGAM RNA, are described hereinbelow with reference to Table 2.

As mentioned hereinabove with reference to FIG. 1, a function of VGAM229 gene, herein designated VGAM is inhibition of expression of VGAM229 target genes. It is appreciated that specific functions, and accordingly utilities, of VGAM229 correlate with, and may be deduced from, the identity of the target genes which VGAM229 binds and inhibits, and the function of these target genes, as elaborated hereinbelow.

Growth Differentiation Factor 8 (GDF8, Accession NM_{—}005259) is a VGAM229 host target gene. GDF8 BINDING SITE is HOST TARGET binding site found in the 3′ untranslated region of mRNA encoded by GDF8, corresponding to a HOST TARGET binding site such as BINDING SITE I, BINDING SITE II or BINDING SITE III. Table 2 illustrates the complementarity of the nucleotide sequences of GDF8 BINDING SITE, designated SEQ ID:1197, to the nucleotide sequence of VGAM229 RNA, herein designated VGAM RNA, also designated SEQ ID:564.

A function of VGAM229 is therefore inhibition of Growth Differentiation Factor 8 (GDF8, Accession NM_{—}005259), a gene which acts specifically as a negative regulator of skeletal muscle growth. Accordingly, utilities of VGAM229 include diagnosis, prevention and treatment of diseases and clinical conditions associated with GDF8. The function of GDF8 and its association with various diseases and clinical conditions, has been established by previous studies, as described hereinabove with reference to VGAM213. Deiodinase, Iodothyronine, Type II (DIO2, Accession NM_{—}000793) is another VGAM229 host target gene. DIO2 BINDING SITE1 and DIO2 BINDING SITE2 are HOST TARGET binding sites found in untranslated regions of mRNA encoded by DIO2, corresponding to HOST TARGET binding sites such as BINDING SITE I, BINDING SITE II or BINDING SITE III. Table 2 illustrates the complementarity of the nucleotide sequences of D102 BINDING SITE1 and D102 BINDING SITE2, designated SEQ ID:773 and SEQ ID: 1454 respectively, to the nucleotide sequence of VGAM229 RNA, herein designated VGAM RNA, also designated SEQ ID:564.

Another function of VGAM229 is therefore inhibition of Deiodinase, Iodothyronine, Type II (D102, Accession NM_{—}000793). Accordingly, utilities of VGAM229 include diagnosis, prevention and treatment of diseases and clinical conditions associated with D102. KIAA0478 (Accession NM_{—}014870) is another VGAM229 host target gene. KIAA0478 BINDING SITE is HOST TARGET binding site found in the 3′ untranslated region of mRNA encoded by KIAA0478, corresponding to a HOST TARGET binding site such as BINDING SITE I, BINDING SITE II or BINDING SITE III. Table 2 illustrates the complementarity of the nucleotide sequences of KIAA0478 BINDING SITE, designated SEQ ID:1573, to the nucleotide sequence of VGAM229 RNA, herein designated VGAM RNA, also designated SEQ ID:564.

Another function of VGAM229 is therefore inhibition of KIAA0478 (Accession NM_{—}014870). Accordingly, utilities of VGAM229 include diagnosis, prevention and treatment of diseases and clinical conditions associated with KIAA0478. KIAA0513 (Accession NM_{—}014732) is another VGAM229 host target gene. KIAA0513 BINDING SITE is HOST TARGET binding site found in the 3′ untranslated region of mRNA encoded by KIAA0513, corresponding to a HOST TARGET binding site such as BINDING SITE I, BINDING SITE II or BINDING SITE III. Table 2 illustrates the complementarity of the nucleotide sequences of KIAA0513 BINDING SITE, designated SEQ ID:1535, to the nucleotide sequence of VGAM229 RNA, herein designated VGAM RNA, also designated SEQ ID:564.

Another function of VGAM229 is therefore inhibition of KIAA0513 (Accession NM_{—}014732). Accordingly, utilities of VGAM229 include diagnosis, prevention and treatment of diseases and clinical conditions associated with KIAA0513. KIAA1323 (Accession XM_{—}032146) is another VGAM229 host target gene. KIAA1323 BINDING SITE is HOST TARGET binding site found in the 5′ untranslated region of mRNA encoded by KIAA1323, corresponding to a HOST TARGET binding site such as BINDING SITE I, BINDING SITE II or BINDING SITE III. Table 2 illustrates the complementarity of the nucleotide sequences of KIAA1323 BINDING SITE, designated SEQ ID:2647, to the nucleotide sequence of VGAM229 RNA, herein designated VGAM RNA, also designated SEQ ID:564.

Another function of VGAM229 is therefore inhibition of KIAA1323 (Accession XM_{—}032146). Accordingly, utilities of VGAM229 include diagnosis, prevention and treatment of diseases and clinical conditions associated with KIAA1323. P450RAI-2 (Accession NM_{—}019885) is another VGAM229 host target gene. P450RAI-2 BINDING SITE is HOST TARGET binding site found in the 3′ untranslated region of mRNA encoded by P450RAI-2, corresponding to a HOST TARGET binding site such as BINDING SITE I, BINDING SITE II or BINDING SITE III. Table 2 illustrates the complementarity of the nucleotide sequences of P450RAI-2 BINDING SITE, designated SEQ ID:1888, to the nucleotide sequence of VGAM229 RNA, herein designated VGAM RNA, also designated SEQ ID:564.

Another function of VGAM229 is therefore inhibition of P450RAI-2 (Accession NM_{—}019885). Accordingly, utilities of VGAM229 include diagnosis, prevention and treatment of diseases and clinical conditions associated with P450RAI-2. Xylosyltransferase I (XYLT1, Accession XM_{—}085432) is another VGAM229 host target gene. XYLT1 BINDING SITE is HOST TARGET binding site found in the 3′ untranslated region of mRNA encoded by XYLT1, corresponding to a HOST TARGET binding site such as BINDING SITE I, BINDING SITE II or BINDING SITE III. Table 2 illustrates the complementarity of the nucleotide sequences of XYLT1 BINDING SITE, designated SEQ ID:3089, to the nucleotide sequence of VGAM229 RNA, herein designated VGAM RNA, also designated SEQ ID:564.

Another function of VGAM229 is therefore inhibition of Xylosyltransferase I (XYLT1, Accession XM_{—}085432). Accordingly, utilities of VGAM229 include diagnosis, prevention and treatment of diseases and clinical conditions associated with XYLT1. LOC143282 (Accession XM_{—}084481) is another VGAM229 host target gene. LOC143282 BINDING SITE is HOST TARGET binding site found in the 3′ untranslated region of mRNA encoded by LOC143282, corresponding to a HOST TARGET binding site such as BINDING SITE I, BINDING SITE II or BINDING SITE III. Table 2 illustrates the complementarity of the nucleotide sequences of LOC143282 BINDING SITE, designated SEQ ID:3055, to the nucleotide sequence of VGAM229 RNA, herein designated VGAM RNA, also designated SEQ ID:564.

Another function of VGAM229 is therefore inhibition of LOC143282 (Accession XM_{—}084481). Accordingly, utilities of VGAM229 include diagnosis, prevention and treatment of diseases and clinical conditions associated with LOC143282. LOC200132 (Accession XM_{—}114126) is another VGAM229 host target gene. LOC200132 BINDING SITE is HOST TARGET binding site found in the 3′ untranslated region of mRNA encoded by LOC200132, corresponding to a HOST TARGET binding site such as BINDING SITE I, BINDING SITE II or BINDING SITE III. Table 2 illustrates the complementarity of the nucleotide sequences of LOC200132 BINDING SITE, designated SEQ ID:3438, to the nucleotide sequence of VGAM229 RNA, herein designated VGAM RNA, also designated SEQ ID:564.

Another function of VGAM229 is therefore inhibition of LOC200132 (Accession XM_{—}114126). Accordingly, utilities of VGAM229 include diagnosis, prevention and treatment of diseases and clinical conditions associated with LOC200132. FIG. 1 further provides a conceptual description of a novel bioinformatically detected viral gene of the present invention, referred to here as Viral Genomic Address Messenger 230 (VGAM230) viral gene, which modulates expression of respective host target genes thereof, the function and utility of which host target genes is known in the art.

VGAM230 is a novel bioinformatically detected regulatory, non protein coding, viral micro RNA (miRNA) gene. The method by which VGAM230 was detected is described hereinabove with reference to FIGS. 1-8.

VGAM230 gene, herein designated VGAM GENE, is a viral gene contained in the genome of Vaccinia Virus. VGAM230 host target gene, herein designated VGAM HOST TARGET GENE, is a human gene contained in the human genome.

VGAM230 gene encodes a VGAM230 precursor RNA, herein designated VGAM PRECURSOR RNA. Similar to other miRNA genes, and unlike most ordinary genes, VGAM230 precursor RNA does not encode a protein. A nucleotide sequence identical or highly similar to the nucleotide sequence of VGAM230 precursor RNA is designated SEQ ID:216, and is provided hereinbelow with reference to the sequence listing part. Nucleotide sequence SEQ ID:216 is located at position 125121 relative to the genome of Vaccinia Virus.

VGAM230 precursor RNA folds onto itself, forming VGAM230 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM230 folded precursor RNA into VGAM230 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM230 RNA is designated SEQ ID:565, and is provided hereinbelow with reference to the sequence listing part.

VGAM230 host target gene, herein designated VGAM HOST TARGET GENE, encodes a corresponding messenger RNA, VGAM230 host target RNA, herein designated VGAM HOST TARGET RNA. VGAM230 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM230 RNA, herein designated VGAM RNA, binds complementarily to one or more host target binding sites located in untranslated regions of VGAM230 host target RNA, herein designated VGAM HOST TARGET RNA. This complementary binding is due to the fact that the nucleotide sequence of VGAM230 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM230 RNA, herein designated VGAM RNA, may have a different number of host target binding sites in untranslated regions of a VGAM230 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM230 RNA, herein designated VGAM RNA, to host target binding sites on VGAM230 host target RNA, herein designated VGAM HOST TARGET RNA, such as BINDING SITE I, BINDING SITE II and BINDING SITE III, inhibits translation of VGAM230 host target RNA into VGAM230 host target protein, herein designated VGAM HOST TARGET PROTEIN. VGAM host target protein is therefore outlined by a broken line.

It is appreciated that VGAM230 host target gene, herein designated VGAM HOST TARGET GENE, in fact represents a plurality of VGAM230 host target genes. The mRNA of each one of this plurality of VGAM230 host target genes comprises one or more host target binding sites, each having a nucleotide sequence which is at least partly complementary to VGAM230 RNA, herein designated VGAM RNA, and which when bound by VGAM230 RNA causes inhibition of translation of respective one or more VGAM230 host target proteins.

It is further appreciated by one skilled in the art that the mode of translational inhibition illustrated by FIG. 1 with specific reference to translational inhibition exerted by VGAM230 gene, herein designated VGAM GENE, on one or more VGAM230 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM230 is inhibition of expression of host target genes, as part of a novel viral mechanism of attacking a host. Accordingly, utilities of VGAM230 include diagnosis, prevention and treatment of viral infection by Vaccinia Virus. Specific functions, and accordingly utilities, of VGAM230 correlate with, and may be deduced from, the identity of the host target genes which VGAM230 binds and inhibits, and the function of these host target genes, as elaborated hereinbelow.

Nucleotide sequences of the VGAM230 precursor RNA, herein designated VGAM PRECURSOR RNA, and of the ‘diced’ VGAM230 RNA, herein designated VGAM RNA, and a schematic representation of the secondary folding of VGAM230 folded precursor RNA, herein designated VGAM FOLDED PRECURSOR RNA, of VGAM230 are further described hereinbelow with reference to Table 1.

Nucleotide sequences of host target binding sites, such as BINDING SITE-I, BINDING SITE-II and BINDING SITE-III of FIG. 1, found on VGAM230 host target RNA, and schematic representation of the complementarity of each of these host target binding sites to VGAM230 RNA, herein designated VGAM RNA, are described hereinbelow with reference to Table 2.

As mentioned hereinabove with reference to FIG. 1, a function of VGAM230 gene, herein designated VGAM is inhibition of expression of VGAM230 target genes. It is appreciated that specific functions, and accordingly utilities, of VGAM230 correlate with, and may be deduced from, the identity of the target genes which VGAM230 binds and inhibits, and the function of these target genes, as elaborated hereinbelow.

Fibroblast Growth Factor Receptor 1 (fms-related tyrosine kinase 2, Pfeiffer syndrome) (FGFR1, Accession NM_{—}000604) is a VGAM230 host target gene. FGFR1 BINDING SITE1 through FGFR1 BINDING SITE6 are HOST TARGET binding sites found in untranslated regions of mRNA encoded by FGFR1, corresponding to HOST TARGET binding sites such as BINDING SITE I, BINDING SITE II or BINDING SITE III. Table 2 illustrates the complementarity of the nucleotide sequences of FGFR1 BINDING SITE1 through FGFR1 BINDING SITE6, designated SEQ ID:759, SEQ ID:1647, SEQ ID:2031, SEQ ID:2033, SEQ ID:2035 and SEQ ID:2036 respectively, to the nucleotide sequence of VGAM230 RNA, herein designated VGAM RNA, also designated SEQ ID:565.

A function of VGAM230 is therefore inhibition of Fibroblast Growth Factor Receptor 1 (fms-related tyrosine kinase 2, Pfeiffer syndrome) (FGFR1, Accession NM_{—}000604). Accordingly, utilities of VGAM230 include diagnosis, prevention and treatment of diseases and clinical conditions associated with FGFR1. Proprotein Convertase Subtilisin/kexin Type 2 (PCSK2, Accession NM_{—}002594) is another VGAM230 host target gene. PCSK2 BINDING SITE is HOST TARGET binding site found in the 3′ untranslated region of mRNA encoded by PCSK2, corresponding to a HOST TARGET binding site such as BINDING SITE I, BINDING SITE II or BINDING SITE III. Table 2 illustrates the complementarity of the nucleotide sequences of PCSK2 BINDING SITE, designated SEQ ID:940, to the nucleotide sequence of VGAM230 RNA, herein designated VGAM RNA, also designated SEQ ID:565.

Another function of VGAM230 is therefore inhibition of Proprotein Convertase Subtilisin/kexin Type 2 (PCSK2, Accession NM_{—}002594), a gene which is involved in the processing of hormone and other protein precursors at sites comprised of pairs of basic amino acid residues. Accordingly, utilities of VGAM230 include diagnosis, prevention and treatment of diseases and clinical conditions associated with PCSK2. The function of PCSK2 has been established by previous studies. Ohagi et al. (1992) observed expression of the chloramphenicol acetyltransferase reporter fusions containing the putative promoter region in insulinoma cells but not in hepatoma cells, consistent with the known tissue-specific pattern of expression of the PC2 gene. Analysis of the level of CAT activity with several deletion mutants identified the region from −1100 to −539 from the translation start site as essential for PC2 promoter activity. During secretion, immature 75-kD proPC2 is cleaved to produce active 68-kD PC2 protein. Taylor et al. (1997) examined the secretion of the proPC2 peptide from COS cells. They found that cleavage was not necessary for PC2 secretion. However, proper folding of the protein was necessary for secretion, and sequences at the C-terminus appeared to stabilize the correct conformation of the protein. Animal model experiments lend further support to the function of PCSK2. Homozygous Pc2-null mice grow normally and are generally healthy but have altered carbohydrate metabolism characterized by mild hypoglycemia and flattened glucose-tolerance curves (Furuta et al., 1997). They also show impaired processing of insulin, glucagon, and somatostatin (Furuta et al., 1998). Furuta et al. (2001) examined the biosynthesis and processing of proglucagon in isolated islets from these mice via pulse-chase labeling and found that proglucagon undergoes essentially no processing in chase periods up to 8 hours. Ultrastructural and immunocytochemical studies indicated the presence of large amounts of proglucagon in atypical-appearing secretory granules in the hyperplastic and hypertrophic A-cells, along with morphological evidence of high rates of proglucagon secretion in Pc2-null mice.

It is appreciated that the abovementioned animal model for PCSK2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Furuta, M.; Zhou, A.; Webb, G.; Carroll, R.; Ravazzola, M.; Orci, L.; Steiner, D. F.: Severe defect in proglucagon processing in islet A-cells of prohormone convertase 2 null mice. J. Biol. Chem. 276: 27197-27202, 2001.; and
Furuta, M.; Carroll, R.; Martin, S.; Swift, H.; Ravazzola, M.; Orci, L.; Steiner, D.: Incomplete processing of proinsulin to insulin accompanied by elevation of Des-31,32 proinsulin in.

Further studies establishing the function and utilities of PCSK2 are found in John Hopkins OMIM database record ID 162151, and in sited publications numbered 844-416, 420-41 and 846 listed in the bibliography section hereinbelow, which are also hereby incorporated by reference. Serine/threonine Kinase 24 (STE20 homolog, yeast) (STK24, Accession NM_{—}003576) is another VGAM230 host target gene. STK24 BINDING SITE is HOST TARGET binding site found in the 3′ untranslated region of mRNA encoded by STK24, corresponding to a HOST TARGET binding site such as BINDING SITE I, BINDING SITE II or BINDING SITE III. Table 2 illustrates the complementarity of the nucleotide sequences of STK24 BINDING SITE, designated SEQ ID: 1038, to the nucleotide sequence of VGAM230 RNA, herein designated VGAM RNA, also designated SEQ ID:565.

Another function of VGAM230 is therefore inhibition of Serine/threonine Kinase 24 (STE20 homolog, yeast) (STK24, Accession NM_{—}003576), a gene which acts on both serine and threonine residues. Accordingly, utilities of VGAM230 include diagnosis, prevention and treatment of diseases and clinical conditions associated with STK24. The function of STK24 has been established by previous studies. Using PCR with degenerate primers based on conserved regions of the kinase domains of STE20 and p65PAK, Schinkmann and Blenis (1997) obtained a fragment from a HeLa cell cDNA library. By screening T cell and HeLa cell cDNA libraries using the fragment as the probe, they isolated a cDNA encoding STK24, which they termed MST3. Sequence analysis showed that the predicted 431-amino acid STK24 protein contains an N-terminal kinase domain and a C-terminal regulatory domain. STK24 shares 69% amino acid identity with STK25 (OMIM Ref. No. 602255) and is a member of the GCK sub family of STE20-like proteins. Northern blot analysis revealed ubiquitous expression of a 2.0-kb STK24 transcript, with highest levels detected in heart, skeletal muscle, and pancreas. Western blot analysis detected a 52-kD STK24 protein in all cell lines tested. In vitro kinase assays demonstrated that STK24 readily phosphorylates MBP (OMIM Ref. No. 159430), histone H3, and itself but not histones H1, H2, and H4 (see OMIM Ref. No. 142711), casein (see OMIM Ref. No. 115450), or phosvitin (CSNK2B; 115441); phosphorylation occurs with manganese as the cofactor. Immunofluorescence microscopy showed that STK24 is expressed in the cytoplasm. By searching an EST database using the conserved catalytic domain of STE20 as the probe, Zhou et al. (2000) identified an STK24 isoform that they called MST3B. The sequence of MST3B is identical after nucleotide 223 to the MST3 sequence reported by Schinkmann and Blenis (1997). MST3B encodes a predicted 443-amino acid protein. RT-PCR and Northern blot analyses revealed that expression of the 2.5-kb MST3B transcript is restricted to brain; Western blot analysis confirmed the brain-specific expression. In situ hybridization analysis showed that MST3B is widely expressed in different brain regions, with high levels in hippocampus, cerebral cortex, and hypothalamus, and moderate levels in geniculate nucleus and thalamic nucleus. Reduced MST3B expression was observed in cerebellum. Functional analyses demonstrated that MST3 but not MST3B phosphorylates and activates p42MAPK (MAPK1; 176948)/p44MAPK (MAPK3; 601795) but not MAPK14 or JNK. Mutational analysis indicated that protein kinase A (see OMIM Ref. No. 176911) phosphorylation of thr18 at the N terminus of MST3B negatively regulates its ability to phosphorylate and activate MAPK3.

Full details of the abovementioned studies are described in the following publications, the disclosure of which are hereby incorporated by reference:
Schinkmann, K.; Blenis, J.: Cloning and characterization of a human STE20-like protein kinase with unusual cofactor requirements. J. Biol. Chem. 272: 28695-28703, 1997.; and
Zhou, T.-H.; Ling, K.; Guo, J.; Zhou, H.; Wu, Y.-L.; Jing, Q.; Ma, L.; Pei, G.: Identification of a human brain-specific isoform of mammalian STE20-like kinase 3 that is regulated by c.

Further studies establishing the function and utilities of STK24 are found in John Hopkins OMIM database record ID 604984, and in sited publications numbered 1622-1623 listed in the bibliography section hereinbelow, which are also hereby incorporated by reference. Tissue Inhibitor of Metalloproteinase 3 (Sorsby fundus dystrophy, pseudoinflammatory) (TIMP3, Accession NM_{—}000362) is another VGAM230 host target gene. TIMP3 BINDING SITE is HOST TARGET binding site found in the 5′ untranslated region of mRNA encoded by TIMP3, corresponding to a HOST TARGET binding site such as BINDING SITE I, BINDING SITE II or BINDING SITE III. Table 2 illustrates the complementarity of the nucleotide sequences of TIMP3 BINDING SITE, designated SEQ ID:733, to the nucleotide sequence of VGAM230 RNA, herein designated VGAM RNA, also designated SEQ ID:565.

Another function of VGAM230 is therefore inhibition of Tissue Inhibitor of Metalloproteinase 3 (Sorsby fundus dystrophy, pseudoinflammatory) (TIMP3, Accession NM_{—}000362). Accordingly, utilities of VGAM230 include diagnosis, prevention and treatment of diseases and clinical conditions associated with TIMP3. AF038169 (Accession NM_{—}013310) is another VGAM230 host target gene. AF038169 BINDING SITE is HOST TARGET binding site found in the 3′ untranslated region of mRNA encoded by AF038169, corresponding to a HOST TARGET binding site such as BINDING SITE I, BINDING SITE II or BINDING SITE III. Table 2 illustrates the complementarity of the nucleotide sequences of AF038169 BINDING SITE, designated SEQ ID: 1440, to the nucleotide sequence of VGAM230 RNA, herein designated VGAM RNA, also designated SEQ ID:565.

Another function of VGAM230 is therefore inhibition of AF038169 (Accession NM_{—}013310). Accordingly, utilities of VGAM230 include diagnosis, prevention and treatment of diseases and clinical conditions associated with AF038169. CRTAM (Accession NM_{—}019604) is another VGAM230 host target gene. CRTAM BINDING SITE is HOST TARGET binding site found in the 3′ untranslated region of mRNA encoded by CRTAM, corresponding to a HOST TARGET binding site such as BINDING SITE I, BINDING SITE II or BINDING SITE III. Table 2 illustrates the complementarity of the nucleotide sequences of CRTAM BINDING SITE, designated SEQ ID:1883, to the nucleotide sequence of VGAM230 RNA, herein designated VGAM RNA, also designated SEQ ID:565.

Another function of VGAM230 is therefore inhibition of CRTAM (Accession NM_{—}019604). Accordingly, utilities of VGAM230 include diagnosis, prevention and treatment of diseases and clinical conditions associated with CRTAM. DKFZP564J0863 (Accession XM_{—}043151) is another VGAM230 host target gene. DKFZP564J0863 BINDING SITE is HOST TARGET binding site found in the 3′ untranslated region of mRNA encoded by DKFZP564J0863, corresponding to a HOST TARGET binding site such as BINDING SITE I, BINDING SITE II or BINDING SITE III. Table 2 illustrates the complementarity of the nucleotide sequences of DKFZP564J0863 BINDING SITE, designated SEQ ID:2815, to the nucleotide sequence of VGAM230 RNA, herein designated VGAM RNA, also designated SEQ ID:565.

Another function of VGAM230 is therefore inhibition of DKFZP564J0863 (Accession XM_{—}043151). Accordingly, utilities of VGAM230 include diagnosis, prevention and treatment of diseases and clinical conditions associated with DKFZP564J0863. FLJ14297 (Accession NM_{—}024903) is another VGAM230 host target gene. FLJ14297 BINDING SITE is HOST TARGET binding site found in the 5′ untranslated region of mRNA encoded by FLJ14297, corresponding to a HOST TARGET binding site such as BINDING SITE I, BINDING SITE II or BINDING SITE III. Table 2 illustrates the complementarity of the nucleotide sequences of FLJ14297 BINDING SITE, designated SEQ ID:2108, to the nucleotide sequence of VGAM230 RNA, herein designated VGAM RNA, also designated SEQ ID:565.

Another function of VGAM230 is therefore inhibition of FLJ14297 (Accession NM_{—}024903). Accordingly, utilities of VGAM230 include diagnosis, prevention and treatment of diseases and clinical conditions associated with FLJ14297. FLJ20508 (Accession NM_{—}017850) is another VGAM230 host target gene. FLJ20508 BINDING SITE is HOST TARGET binding site found in the 3′ untranslated region of mRNA encoded by FLJ20508, corresponding to a HOST TARGET binding site such as BINDING SITE I, BINDING SITE II or BINDING SITE III. Table 2 illustrates the complementarity of the nucleotide sequences of FLJ20508 BINDING SITE, designated SEQ ID:1757, to the nucleotide sequence of VGAM230 RNA, herein designated VGAM RNA, also designated SEQ ID:565.

Another function of VGAM230 is therefore inhibition of FLJ20508 (Accession NM_{—}017850). Accordingly, utilities of VGAM230 include diagnosis, prevention and treatment of diseases and clinical conditions associated with FLJ20508. FLJ23462 (Accession NM_{—}024843) is another VGAM230 host target gene. FLJ23462 BINDING SITE is HOST TARGET binding site found in the 3′ untranslated region of mRNA encoded by FLJ23462, corresponding to a HOST TARGET binding site such as BINDING SITE I, BINDING SITE II or BINDING SITE III. Table 2 illustrates the complementarity of the nucleotide sequences of FLJ23462 BINDING SITE, designated SEQ ID:2096, to the nucleotide sequence of VGAM230 RNA, herein designated VGAM RNA, also designated SEQ ID:565.

Another function of VGAM230 is therefore inhibition of FLJ23462 (Accession NM_{—}024843). Accordingly, utilities of VGAM230 include diagnosis, prevention and treatment of diseases and clinical conditions associated with FLJ23462. LOC126167 (Accession XM_{—}058997) is another VGAM230 host target gene. LOC126167 BINDING SITE is HOST TARGET binding site found in the 3′ untranslated region of mRNA encoded by LOC126167, corresponding to a HOST TARGET binding site such as BINDING SITE I, BINDING SITE II or BINDING SITE III. Table 2 illustrates the complementarity of the nucleotide sequences of LOC126167 BINDING SITE, designated SEQ ID:2998, to the nucleotide sequence of VGAM230 RNA, herein designated VGAM RNA, also designated SEQ ID:565.

Another function of VGAM230 is therefore inhibition of LOC126167 (Accession XM_{—}058997). Accordingly, utilities of VGAM230 include diagnosis, prevention and treatment of diseases and clinical conditions associated with LOC126167. LOC157247 (Accession XM_{—}088275) is another VGAM230 host target gene. LOC157247 BINDING SITE is HOST TARGET binding site found in the 5′ untranslated region of mRNA encoded by LOC157247, corresponding to a HOST TARGET binding site such as BINDING SITE I, BINDING SITE II or BINDING SITE III. Table 2 illustrates the complementarity of the nucleotide sequences of LOC157247 BINDING SITE, designated SEQ ID:3200, to the nucleotide sequence of VGAM230 RNA, herein designated VGAM RNA, also designated SEQ ID:565.

Another function of VGAM230 is therefore inhibition of LOC157247 (Accession XM_{—}088275). Accordingly, utilities of VGAM230 include diagnosis, prevention and treatment of diseases and clinical conditions associated with LOC157247. LOC161357 (Accession XM_{—}090827) is another VGAM230 host target gene. LOC161357 BINDING SITE is HOST TARGET binding site found in the 5′ untranslated region of mRNA encoded by LOC161357, corresponding to a HOST TARGET binding site such as BINDING SITE I, BINDING SITE II or BINDING SITE III. Table 2 illustrates the complementarity of the nucleotide sequences of LOC161357 BINDING SITE, designated SEQ ID:3240, to the nucleotide sequence of VGAM230 RNA, herein designated VGAM RNA, also designated SEQ ID:565.

Another function of VGAM230 is therefore inhibition of LOC161357 (Accession XM_{—}090827). Accordingly, utilities of VGAM230 include diagnosis, prevention and treatment of diseases and clinical conditions associated with LOC161357. LOC201965 (Accession XM_{—}114412) is another VGAM230 host target gene. LOC201965 BINDING SITE is HOST TARGET binding site found in the 3′ untranslated region of mRNA encoded by LOC201965, corresponding to a HOST TARGET binding site such as BINDING SITE I, BINDING SITE II or BINDING SITE III. Table 2 illustrates the complementarity of the nucleotide sequences of LOC201965 BINDING SITE, designated SEQ ID:3455, to the nucleotide sequence of VGAM230 RNA, herein designated VGAM RNA, also designated SEQ ID:565.

Another function of VGAM230 is therefore inhibition of LOC201965 (Accession XM_{—}114412). Accordingly, utilities of VGAM230 include diagnosis, prevention and treatment of diseases and clinical conditions associated with LOC201965. LOC253758 (Accession XM_{—}173067) is another VGAM230 host target gene. LOC253758 BINDING SITE is HOST TARGET binding site found in the 5′ untranslated region of mRNA encoded by LOC253758, corresponding to a HOST TARGET binding site such as BINDING SITE I, BINDING SITE II or BINDING SITE III. Table 2 illustrates the complementarity of the nucleotide sequences of LOC253758 BINDING SITE, designated SEQ ID:3723, to the nucleotide sequence of VGAM230 RNA, herein designated VGAM RNA, also designated SEQ ID:565.

Another function of VGAM230 is therefore inhibition of LOC253758 (Accession XM_{—}173067). Accordingly, utilities of VGAM230 include diagnosis, prevention and treatment of diseases and clinical conditions associated with LOC253758. LOC91661 (Accession NM_{—}138372) is another VGAM230 host target gene. LOC91661 BINDING SITE is HOST TARGET binding site found in the 3′ untranslated region of mRNA encoded by LOC91661, corresponding to a HOST TARGET binding site such as BINDING SITE I, BINDING SITE II or BINDING SITE III. Table 2 illustrates the complementarity of the nucleotide sequences of LOC91661 BINDING SITE, designated SEQ ID:2438, to the nucleotide sequence of VGAM230 RNA, herein designated VGAM RNA, also designated SEQ ID:565.

Another function of VGAM230 is therefore inhibition of LOC91661 (Accession NM_{—}138372). Accordingly, utilities of VGAM230 include diagnosis, prevention and treatment of diseases and clinical conditions associated with LOC91661. FIG. 1 further provides a conceptual description of a novel bioinformatically detected viral gene of the present invention, referred to here as Viral Genomic Address Messenger 231 (VGAM231) viral gene, which modulates expression of respective host target genes thereof, the function and utility of which host target genes is known in the art.

VGAM231 is a novel bioinformatically detected regulatory, non protein coding, viral micro RNA (miRNA) gene. The method by which VGAM231 was detected is described hereinabove with reference to FIGS. 1-8.

VGAM231 gene, herein designated VGAM GENE, is a viral gene contained in the genome of Vaccinia Virus. VGAM231 host target gene, herein designated VGAM HOST TARGET GENE, is a human gene contained in the human genome.

VGAM231 gene encodes a VGAM231 precursor RNA, herein designated VGAM PRECURSOR RNA. Similar to other miRNA genes, and unlike most ordinary genes, VGAM231 precursor RNA does not encode a protein. A nucleotide sequence identical or highly similar to the nucleotide sequence of VGAM231 precursor RNA is designated SEQ ID:217, and is provided hereinbelow with reference to the sequence listing part. Nucleotide sequence SEQ ID:217 is located at position 125645 relative to the genome of Vaccinia Virus.

VGAM231 precursor RNA folds onto itself, forming VGAM231 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM231 folded precursor RNA into VGAM231 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM231 RNA is designated SEQ ID:566, and is provided hereinbelow with reference to the sequence listing part.

VGAM231 host target gene, herein designated VGAM HOST TARGET GENE, encodes a corresponding messenger RNA, VGAM231 host target RNA, herein designated VGAM HOST TARGET RNA. VGAM231 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM231 RNA, herein designated VGAM RNA, binds complementarily to one or more host target binding sites located in untranslated regions of VGAM231 host target RNA, herein designated VGAM HOST TARGET RNA. This complementary binding is due to the fact that the nucleotide sequence of VGAM231 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM231 RNA, herein designated VGAM RNA, may have a different number of host target binding sites in untranslated regions of a VGAM231 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM231 RNA, herein designated VGAM RNA, to host target binding sites on VGAM231 host target RNA, herein designated VGAM HOST TARGET RNA, such as BINDING SITE I, BINDING SITE II and BINDING SITE III, inhibits translation of VGAM231 host target RNA into VGAM231 host target protein, herein designated VGAM HOST TARGET PROTEIN. VGAM host target protein is therefore outlined by a broken line.

It is appreciated that VGAM231 host target gene, herein designated VGAM HOST TARGET GENE, in fact represents a plurality of VGAM231 host target genes. The mRNA of each one of this plurality of VGAM231 host target genes comprises one or more host target binding sites, each having a nucleotide sequence which is at least partly complementary to VGAM231 RNA, herein designated VGAM RNA, and which when bound by VGAM231 RNA causes inhibition of translation of respective one or more VGAM231 host target proteins.

It is further appreciated by one skilled in the art that the mode of translational inhibition illustrated by FIG. 1 with specific reference to translational inhibition exerted by VGAM231 gene, herein designated VGAM GENE, on one or more VGAM231 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM231 is inhibition of expression of host target genes, as part of a novel viral mechanism of attacking a host. Accordingly, utilities of VGAM231 include diagnosis, prevention and treatment of viral infection by Vaccinia Virus. Specific functions, and accordingly utilities, of VGAM231 correlate with, and may be deduced from, the identity of the host target genes which VGAM231 binds and inhibits, and the function of these host target genes, as elaborated hereinbelow.

Nucleotide sequences of the VGAM231 precursor RNA, herein designated VGAM PRECURSOR RNA, and of the ‘diced’ VGAM231 RNA, herein designated VGAM RNA, and a schematic representation of the secondary folding of VGAM231 folded precursor RNA, herein designated VGAM FOLDED PRECURSOR RNA, of VGAM231 are further described hereinbelow with reference to Table 1.

Nucleotide sequences of host target binding sites, such as BINDING SITE-I, BINDING SITE-II and BINDING SITE-III of FIG. 1, found on VGAM231 host target RNA, and schematic representation of the complementarity of each of these host target binding sites to VGAM231 RNA, herein designated VGAM RNA, are described hereinbelow with reference to Table 2.

As mentioned hereinabove with reference to FIG. 1, a function of VGAM231 gene, herein designated VGAM is inhibition of expression of VGAM231 target genes. It is appreciated that specific functions, and accordingly utilities, of VGAM231 correlate with, and may be deduced from, the identity of the target genes which VGAM231 binds and inhibits, and the function of these target genes, as elaborated hereinbelow.

Spastic Paraplegia 4 (autosomal dominant; spastin) (SPG4, Accession NM_{—}014946) is a VGAM231 host target gene. SPG4 BINDING SITE is HOST TARGET binding site found in the 3′ untranslated region of mRNA encoded by SPG4, corresponding to a HOST TARGET binding site such as BINDING SITE I, BINDING SITE II or BINDING SITE III. Table 2 illustrates the complementarity of the nucleotide sequences of SPG4 BINDING SITE, designated SEQ ID:1595, to the nucleotide sequence of VGAM231 RNA, herein designated VGAM RNA, also designated SEQ ID:566.

A function of VGAM231 is therefore inhibition of Spastic Paraplegia 4 (autosomal dominant; spastin) (SPG4, Accession NM_{—}014946), a gene which is probably an ATPase involved in the assembly or function of nuclear protein complexes. Accordingly, utilities of VGAM231 include diagnosis, prevention and treatment of diseases and clinical conditions associated with SPG4. The function of SPG4 has been established by previous studies. SPG4 is ubiquitously expressed in human adult and fetal tissue, showing slightly higher expression in fetal brain. Hazan et al. (1999) cloned the mouse ortholog of SPG4, which between amino acids 113 and 616 has 96% identity with human SPG4. Spg4 transcripts are ubiquitously expressed in adult tissues and from embryonic day 7 to 17 in mouse. Interaction with the cytoskeleton was mediated by the N-terminal region of spastin and was regulated through the ATPase activity of the AAA domain. Expression of missense mutations (including 604277.0001, 604277.0002, and 604277.0004) into the AAA domain led to constitutive binding to microtubules in transfected cells and induced the disappearance of the aster and the formation of thick perinuclear bundles, suggesting a role of spastin in microtubule dynamics. Consistently, wildtype spastin promoted microtubule disassembly in transfected cells. The authors suggested that spastin may be involved in microtubule dynamics similarly to the highly homologous microtubule-severing protein katanin (OMIM Ref. No. 606696). The authors hypothesized that impairment of fine regulation of the microtubule cytoskeleton in long axons, due to spastin mutations, may underlie the pathogenesis of hereditary spastic paraplegia

Full details of the abovementioned studies are described in the following publications, the disclosure of which are hereby incorporated by reference:
Hazan, J.; Fonknechten, N.; Mavel, D.; Paternotte, C.; Samson, D.; Artiguenave, F.; Davoine, C.-S.; Cruaud, C.; Durr, A.; Wincker, P.; Brottier, P.; Cattolico, L.; Barbe, V.; Burgunder, J.-M.; Prud'homme, J.-F.; Brice, A.; Fontaine, B.; Heilig, R.; Weissenbach, J.: Spastin, a new AAA protein, is altered in the most frequent form of autosomal dominant spastic paraplegia. Nature Genet. 23: 296-303, 1999.; and
Errico, A.; Ballabio, A.; Rugarli, E. I.: Spastin, the protein mutated in autosomal dominant hereditary spastic paraplegia, is involved in microtubule dynamics. Hum. Molec. Genet. 11: 153.

Further studies establishing the function and utilities of SPG4 are found in John Hopkins OMIM database record ID 604277, and in sited publications numbered 2388-1665, 2389-1670, 1666, 2390-174 and 2391 listed in the bibliography section hereinbelow, which are also hereby incorporated by reference. KIAA0672 (Accession NM_{—}014859) is another VGAM231 host target gene. KIAA0672 BINDING SITE is HOST TARGET binding site found in the 3′ untranslated region of mRNA encoded by KIAA0672, corresponding to a HOST TARGET binding site such as BINDING SITE I, BINDING SITE II or BINDING SITE III. Table 2 illustrates the complementarity of the nucleotide sequences of KIAA0672 BINDING SITE, designated SEQ ID:1569, to the nucleotide sequence of VGAM231 RNA, herein designated VGAM RNA, also designated SEQ ID:566.

Another function of VGAM231 is therefore inhibition of KIAA0672 (Accession NM_{—}014859). Accordingly, utilities of VGAM231 include diagnosis, prevention and treatment of diseases and clinical conditions associated with KIAA0672. MGC32104 (Accession NM_{—}144684) is another VGAM231 host target gene. MGC32104 BINDING SITE is HOST TARGET binding site found in the 3′ untranslated region of mRNA encoded by MGC32104, corresponding to a HOST TARGET binding site such as BINDING SITE I, BINDING SITE II or BINDING SITE III. Table 2 illustrates the complementarity of the nucleotide sequences of MGC32104 BINDING SITE, designated SEQ ID:2495, to the nucleotide sequence of VGAM231 RNA, herein designated VGAM RNA, also designated SEQ ID:566.

Another function of VGAM231 is therefore inhibition of MGC32104 (Accession NM_{—}144684). Accordingly, utilities of VGAM231 include diagnosis, prevention and treatment of diseases and clinical conditions associated with MGC32104. PRO0082 (Accession NM_{—}018590) is another VGAM231 host target gene. PRO0082 BINDING SITE is HOST TARGET binding site found in the 3′ untranslated region of mRNA encoded by PRO0082, corresponding to a HOST TARGET binding site such as BINDING SITE I, BINDING SITE II or BINDING SITE III. Table 2 illustrates the complementarity of the nucleotide sequences of PRO0082 BINDING SITE, designated SEQ ID:1851, to the nucleotide sequence of VGAM231 RNA, herein designated VGAM RNA, also designated SEQ ID:566.

Another function of VGAM231 is therefore inhibition of PRO0082 (Accession NM_{—}018590). Accordingly, utilities of VGAM231 include diagnosis, prevention and treatment of diseases and clinical conditions associated with PRO0082. FIG. 1 further provides a conceptual description of a novel bioinformatically detected viral gene of the present invention, referred to here as Viral Genomic Address Messenger 232 (VGAM232) viral gene, which modulates expression of respective host target genes thereof, the function and utility of which host target genes is known in the art.

VGAM232 is a novel bioinformatically detected regulatory, non protein coding, viral micro RNA (miRNA) gene. The method by which VGAM232 was detected is described hereinabove with reference to FIGS. 1-8.

VGAM232 gene, herein designated VGAM GENE, is a viral gene contained in the genome of Vaccinia Virus. VGAM232 host target gene, herein designated VGAM HOST TARGET GENE, is a human gene contained in the human genome.

VGAM232 gene encodes a VGAM232 precursor RNA, herein designated VGAM PRECURSOR RNA. Similar to other miRNA genes, and unlike most ordinary genes, VGAM232 precursor RNA does not encode a protein. A nucleotide sequence identical or highly similar to the nucleotide sequence of VGAM232 precursor RNA is designated SEQ ID:218, and is provided hereinbelow with reference to the sequence listing part. Nucleotide sequence SEQ ID:218 is located at position 125237 relative to the genome of Vaccinia Virus.

VGAM232 precursor RNA folds onto itself, forming VGAM232 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM232 folded precursor RNA into VGAM232 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM232 RNA is designated SEQ ID:567, and is provided hereinbelow with reference to the sequence listing part.

VGAM232 host target gene, herein designated VGAM HOST TARGET GENE, encodes a corresponding messenger RNA, VGAM232 host target RNA, herein designated VGAM HOST TARGET RNA. VGAM232 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM232 RNA, herein designated VGAM RNA, binds complementarily to one or more host target binding sites located in untranslated regions of VGAM232 host target RNA, herein designated VGAM HOST TARGET RNA. This complementary binding is due to the fact that the nucleotide sequence of VGAM232 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM232 RNA, herein designated VGAM RNA, may have a different number of host target binding sites in untranslated regions of a VGAM232 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM232 RNA, herein designated VGAM RNA, to host target binding sites on VGAM232 host target RNA, herein designated VGAM HOST TARGET RNA, such as BINDING SITE I, BINDING SITE II and BINDING SITE III, inhibits translation of VGAM232 host target RNA into VGAM232 host target protein, herein designated VGAM HOST TARGET PROTEIN. VGAM host target protein is therefore outlined by a broken line.

It is appreciated that VGAM232 host target gene, herein designated VGAM HOST TARGET GENE, in fact represents a plurality of VGAM232 host target genes. The mRNA of each one of this plurality of VGAM232 host target genes comprises one or more host target binding sites, each having a nucleotide sequence which is at least partly complementary to VGAM232 RNA, herein designated VGAM RNA, and which when bound by VGAM232 RNA causes inhibition of translation of respective one or more VGAM232 host target proteins.

It is further appreciated by one skilled in the art that the mode of translational inhibition illustrated by FIG. 1 with specific reference to translational inhibition exerted by VGAM232 gene, herein designated VGAM GENE, on one or more VGAM232 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM232 is inhibition of expression of host target genes, as part of a novel viral mechanism of attacking a host. Accordingly, utilities of VGAM232 include diagnosis, prevention and treatment of viral infection by Vaccinia Virus. Specific functions, and accordingly utilities, of VGAM232 correlate with, and may be deduced from, the identity of the host target genes which VGAM232 binds and inhibits, and the function of these host target genes, as elaborated hereinbelow.

Nucleotide sequences of the VGAM232 precursor RNA, herein designated VGAM PRECURSOR RNA, and of the ‘diced’ VGAM232 RNA, herein designated VGAM RNA, and a schematic representation of the secondary folding of VGAM232 folded precursor RNA, herein designated VGAM FOLDED PRECURSOR RNA, of VGAM232 are further described hereinbelow with reference to Table 1.

Nucleotide sequences of host target binding sites, such as BINDING SITE-I, BINDING SITE-II and BINDING SITE-III of FIG. 1, found on VGAM232 host target RNA, and schematic representation of the complementarity of each of these host target binding sites to VGAM232 RNA, herein designated VGAM RNA, are described hereinbelow with reference to Table 2.

As mentioned hereinabove with reference to FIG. 1, a function of VGAM232 gene, herein designated VGAM is inhibition of expression of VGAM232 target genes. It is appreciated that specific functions, and accordingly utilities, of VGAM232 correlate with, and may be deduced from, the identity of the target genes which VGAM232 binds and inhibits, and the function of these target genes, as elaborated hereinbelow.

Tumor Rejection Antigen (gp96) Pseudogene 1 (TRAP1, Accession NM_{—}016292) is a VGAM232 host target gene. TRAP1 BINDING SITE is HOST TARGET binding site found in the 3′ untranslated region of mRNA encoded by TRAP1, corresponding to a HOST TARGET binding site such as BINDING SITE I, BINDING SITE II or BINDING SITE III. Table 2 illustrates the complementarity of the nucleotide sequences of TRAP1 BINDING SITE, designated SEQ ID:1678, to the nucleotide sequence of VGAM232 RNA, herein designated VGAM RNA, also designated SEQ ID:567.

A function of VGAM232 is therefore inhibition of Tumor Rejection Antigen (gp96) Pseudogene 1 (TRAP1, Accession NM_{—}016292), a gene which chaperone that expresses an atpase activity. Accordingly, utilities of VGAM232 include diagnosis, prevention and treatment of diseases and clinical conditions associated with TRAP1. The function of TRAP1 has been established by previous studies. Using a yeast 2-hybrid screen of a Gal4/HeLa cDNA library with the intracellular domain of TNFR1 (OMIM Ref. No. 191190) as bait, followed by 5-prime RACE, Song et al. (1995) isolated a partial cDNA encoding TRAP1. The deduced 661-amino acid protein is 60% similar to HSP90 family members, although it lacks the highly charged domain found in HSP90 proteins. Northern blot analysis revealed variable but ubiquitous expression of a 2.7-kb TRAP1 transcript. GST pull-down analysis indicated that TRAP1 interacts with the N-terminal half of TNFR1. By yeast 2-hybrid screening of several cDNA libraries with an N-terminally truncated retinoblastoma protein (RB; 180200) as bait, Chen et al. (1996) obtained a nearly complete cDNA encoding TRAP1, which they termed HSP75. HSP75 has 698 amino acids. Immunoprecipitation, immunoblot analysis, and immunofluorescence microscopy demonstrated expression of a 75-kD cytoplasmic protein that colocalizes with RB during mitosis but not during other phases. During heat shock, HSP75 migrates to the nucleus. Binding analysis showed that HSP75 uses an LxCxE motif to bind to the T antigen-binding domains of RB. Western blot analysis indicated that HSP75 is able to refold denatured RB, suggesting that HSP75 acts as a chaperone for RB. In a yeast 2-hybrid screen with exostosin-2 (EXT2; 133701) as bait, Simmons et al. (1999) isolated TRAP1. They reported that the full-length cDNA sequence encodes a 704-amino acid protein (GenBank AF154108). Binding analysis determined that TRAP1 interacts with the C-terminal ends of the proteins encoded by both multiple exostoses-causing genes, EXT1 (OMIM Ref. No. 133700) and EXT2, but not with EXTL1 (OMIM Ref. No. 601738) or EXTL3 (OMIM Ref. No. 605744). The interaction requires the presence of a his residue in the EXT proteins, the loss of which had been identified in a single family with type I multiple exostoses (OMIM Ref. No. 133700) by Raskind et al. (1998). Felts et al. (2000) reported that TRAP1 lacks the chaperone activities of HSP90 and also fails to interact with cochaperones of HSP90. However, TRAP1 does bind ATP and expresses an ATPase activity that can be blocked by the HSP90 inhibitor geldanamycin. Immunofluorescence microscopy demonstrated a mitochondrial localization for TRAP1. TRAP1 possesses a mitochondrial localization sequence, STQTAED, beginning after cleavage at position 59. Sequence analysis predicted that TRAP1 is 54% identical to a Drosophila homolog.

Full details of the abovementioned studies are described in the following publications, the disclosure of which are hereby incorporated by reference:
Felts, S. J.; Owen, B. A. L.; Nguyen, P.; Trepel, J.; Donner, D. B.; Toft, D. O.: The hsp90-related protein TRAP1 is a mitochondrial protein with distinct functional properties. J. Biol. Chem. 275: 3305-3312, 2000.; and
Raskind, W. H.; Conrad, E. U., III; Matsushita, M.; Wijsman, E. M.; Wells, D. E.; Chapman, N.; Sandell, L. J.; Wagner, M.; Houck, J.: Evaluation of locus heterogeneity and EXT1 mutati.

Further studies establishing the function and utilities of TRAP1 are found in John Hopkins OMIM database record ID 606219, and in sited publications numbered 1525-1526, 85 and 2721 listed in the bibliography section hereinbelow, which are also hereby incorporated by reference. ASAH (Accession NM_{—}004315) is another VGAM232 host target gene. ASAH BINDING SITE is HOST TARGET binding site found in the 3′ untranslated region of mRNA encoded by ASAH, corresponding to a HOST TARGET binding site such as BINDING SITE I, BINDING SITE II or BINDING SITE III. Table 2 illustrates the complementarity of the nucleotide sequences of ASAH BINDING SITE, designated SEQ ID: 1098, to the nucleotide sequence of VGAM232 RNA, herein designated VGAM RNA, also designated SEQ ID:567.

Another function of VGAM232 is therefore inhibition of ASAH (Accession NM_{—}004315). Accordingly, utilities of VGAM232 include diagnosis, prevention and treatment of diseases and clinical conditions associated with ASAH. Cyclin M1 (CNNM1, Accession NM_{—}020348) is another VGAM232 host target gene. CNNM1 BINDING SITE is HOST TARGET binding site found in the 3′ untranslated region of mRNA encoded by CNNM1, corresponding to a HOST TARGET binding site such as BINDING SITE I, BINDING SITE II or BINDING SITE III. Table 2 illustrates the complementarity of the nucleotide sequences of CNNM1 BINDING SITE, designated SEQ ID:1913, to the nucleotide sequence of VGAM232 RNA, herein designated VGAM RNA, also designated SEQ ID:567.

Another function of VGAM232 is therefore inhibition of Cyclin M1 (CNNM1, Accession NM_{—}020348). Accordingly, utilities of VGAM232 include diagnosis, prevention and treatment of diseases and clinical conditions associated with CNNM1. Rab11-FIP2 (Accession NM_{—}014904) is another VGAM232 host target gene. Rab11-FIP2 BINDING SITE is HOST TARGET binding site found in the 3′ untranslated region of mRNA encoded by Rab11-FIP2, corresponding to a HOST TARGET binding site such as BINDING SITE I, BINDING SITE II or BINDING SITE III. Table 2 illustrates the complementarity of the nucleotide sequences of Rab11-FIP2 BINDING SITE, designated SEQ ID:1582, to the nucleotide sequence of VGAM232 RNA, herein designated VGAM RNA, also designated SEQ ID:567.

Another function of VGAM232 is therefore inhibition of Rab11-FIP2 (Accession NM_{—}014904). Accordingly, utilities of VGAM232 include diagnosis, prevention and treatment of diseases and clinical conditions associated with Rab11-FIP2. Ring Finger Protein 38 (RNF38, Accession NM_{—}022781) is another VGAM232 host target gene. RNF38 BINDING SITE is HOST TARGET binding site found in the 3′ untranslated region of mRNA encoded by RNF38, corresponding to a HOST TARGET binding site such as BINDING SITE I, BINDING SITE II or BINDING SITE III. Table 2 illustrates the complementarity of the nucleotide sequences of RNF38 BINDING SITE, designated SEQ ID:2009, to the nucleotide sequence of VGAM232 RNA, herein designated VGAM RNA, also designated SEQ ID:567.

Another function of VGAM232 is therefore inhibition of Ring Finger Protein 38 (RNF38, Accession NM_{—}022781). Accordingly, utilities of VGAM232 include diagnosis, prevention and treatment of diseases and clinical conditions associated with RNF38. LOC152283 (Accession XM_{—}098196) is another VGAM232 host target gene. LOC152283 BINDING SITE is HOST TARGET binding site found in the 3′ untranslated region of mRNA encoded by LOC152283, corresponding to a HOST TARGET binding site such as BINDING SITE I, BINDING SITE II or BINDING SITE III. Table 2 illustrates the complementarity of the nucleotide sequences of LOC152283 BINDING SITE, designated SEQ ID:3357, to the nucleotide sequence of VGAM232 RNA, herein designated VGAM RNA, also designated SEQ ID:567.

Another function of VGAM232 is therefore inhibition of LOC152283 (Accession XM_{—}098196). Accordingly, utilities of VGAM232 include diagnosis, prevention and treatment of diseases and clinical conditions associated with LOC152283. LOC160484 (Accession XM_{—}090326) is another VGAM232 host target gene. LOC160484 BINDING SITE is HOST TARGET binding site found in the 5′ untranslated region of mRNA encoded by LOC160484, corresponding to a HOST TARGET binding site such as BINDING SITE I, BINDING SITE II or BINDING SITE III. Table 2 illustrates the complementarity of the nucleotide sequences of LOC160484 BINDING SITE, designated SEQ ID:3239, to the nucleotide sequence of VGAM232 RNA, herein designated VGAM RNA, also designated SEQ ID:567.

Another function of VGAM232 is therefore inhibition of LOC160484 (Accession XM_{—}090326). Accordingly, utilities of VGAM232 include diagnosis, prevention and treatment of diseases and clinical conditions associated with LOC160484. LOC90119 (Accession XM_{—}029167) is another VGAM232 host target gene. LOC90119 BINDING SITE is HOST TARGET binding site found in the 3′ untranslated region of mRNA encoded by LOC90119, corresponding to a HOST TARGET binding site such as BINDING SITE I, BINDING SITE II or BINDING SITE III. Table 2 illustrates the complementarity of the nucleotide sequences of LOC90119 BINDING SITE, designated SEQ ID:2600, to the nucleotide sequence of VGAM232 RNA, herein designated VGAM RNA, also designated SEQ ID:567.

Another function of VGAM232 is therefore inhibition of LOC90119 (Accession XM_{—}029167). Accordingly, utilities of VGAM232 include diagnosis, prevention and treatment of diseases and clinical conditions associated with LOC90119. FIG. 1 further provides a conceptual description of a novel bioinformatically detected viral gene of the present invention, referred to here as Viral Genomic Address Messenger 233 (VGAM233) viral gene, which modulates expression of respective host target genes thereof, the function and utility of which host target genes is known in the art.

VGAM233 is a novel bioinformatically detected regulatory, non protein coding, viral micro RNA (miRNA) gene. The method by which VGAM233 was detected is described hereinabove with reference to FIGS. 1-8.

VGAM233 gene, herein designated VGAM GENE, is a viral gene contained in the genome of Vaccinia Virus. VGAM233 host target gene, herein designated VGAM HOST TARGET GENE, is a human gene contained in the human genome.

VGAM233 gene encodes a VGAM233 precursor RNA, herein designated VGAM PRECURSOR RNA. Similar to other miRNA genes, and unlike most ordinary genes, VGAM233 precursor RNA does not encode a protein. A nucleotide sequence identical or highly similar to the nucleotide sequence of VGAM233 precursor RNA is designated SEQ ID:219, and is provided hereinbelow with reference to the sequence listing part. Nucleotide sequence SEQ ID:219 is located at position 125406 relative to the genome of Vaccinia Virus.

VGAM233 precursor RNA folds onto itself, forming VGAM233 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM233 folded precursor RNA into VGAM233 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM233 RNA is designated SEQ ID:568, and is provided hereinbelow with reference to the sequence listing part.

VGAM233 host target gene, herein designated VGAM HOST TARGET GENE, encodes a corresponding messenger RNA, VGAM233 host target RNA, herein designated VGAM HOST TARGET RNA. VGAM233 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM233 RNA, herein designated VGAM RNA, binds complementarily to one or more host target binding sites located in untranslated regions of VGAM233 host target RNA, herein designated VGAM HOST TARGET RNA. This complementary binding is due to the fact that the nucleotide sequence of VGAM233 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM233 RNA, herein designated VGAM RNA, may have a different number of host target binding sites in untranslated regions of a VGAM233 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM233 RNA, herein designated VGAM RNA, to host target binding sites on VGAM233 host target RNA, herein designated VGAM HOST TARGET RNA, such as BINDING SITE I, BINDING SITE II and BINDING SITE III, inhibits translation of VGAM233 host target RNA into VGAM233 host target protein, herein designated VGAM HOST TARGET PROTEIN. VGAM host target protein is therefore outlined by a broken line.

It is appreciated that VGAM233 host target gene, herein designated VGAM HOST TARGET GENE, in fact represents a plurality of VGAM233 host target genes. The mRNA of each one of this plurality of VGAM233 host target genes comprises one or more host target binding sites, each having a nucleotide sequence which is at least partly complementary to VGAM233 RNA, herein designated VGAM RNA, and which when bound by VGAM233 RNA causes inhibition of translation of respective one or more VGAM233 host target proteins.

It is further appreciated by one skilled in the art that the mode of translational inhibition illustrated by FIG. 1 with specific reference to translational inhibition exerted by VGAM233 gene, herein designated VGAM GENE, on one or more VGAM233 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM233 is inhibition of expression of host target genes, as part of a novel viral mechanism of attacking a host. Accordingly, utilities of VGAM233 include diagnosis, prevention and treatment of viral infection by Vaccinia Virus. Specific functions, and accordingly utilities, of VGAM233 correlate with, and may be deduced from, the identity of the host target genes which VGAM233 binds and inhibits, and the function of these host target genes, as elaborated hereinbelow.

Nucleotide sequences of the VGAM233 precursor RNA, herein designated VGAM PRECURSOR RNA, and of the ‘diced’ VGAM233 RNA, herein designated VGAM RNA, and a schematic representation of the secondary folding of VGAM233 folded precursor RNA, herein designated VGAM FOLDED PRECURSOR RNA, of VGAM233 are further described hereinbelow with reference to Table 1.

Nucleotide sequences of host target binding sites, such as BINDING SITE-I, BINDING SITE-II and BINDING SITE-III of FIG. 1, found on VGAM233 host target RNA, and schematic representation of the complementarity of each of these host target binding sites to VGAM233 RNA, herein designated VGAM RNA, are described hereinbelow with reference to Table 2.

As mentioned hereinabove with reference to FIG. 1, a function of VGAM233 gene, herein designated VGAM is inhibition of expression of VGAM233 target genes. It is appreciated that specific functions, and accordingly utilities, of VGAM233 correlate with, and may be deduced from, the identity of the target genes which VGAM233 binds and inhibits, and the function of these target genes, as elaborated hereinbelow.

Myotubularin Related Protein 2 (MTMR2, Accession NM_{—}016156) is a VGAM233 host target gene. MTMR2 BINDING SITE is HOST TARGET binding site found in the 3′ untranslated region of mRNA encoded by MTMR2, corresponding to a HOST TARGET binding site such as BINDING SITE I, BINDING SITE II or BINDING SITE III. Table 2 illustrates the complementarity of the nucleotide sequences of MTMR2 BINDING SITE, designated SEQ ID: 1664, to the nucleotide sequence of VGAM233 RNA, herein designated VGAM RNA, also designated SEQ ID:568.

A function of VGAM233 is therefore inhibition of Myotubularin Related Protein 2 (MTMR2, Accession NM_{—}016156). Accordingly, utilities of VGAM233 include diagnosis, prevention and treatment of diseases and clinical conditions associated with MTMR2. Proto-cadherin Gamma Sub family A, 8 (PCDHGA8, Accession NM_{—}032088) is another VGAM233 host target gene. PCDHGA8 BINDING SITE1 and PCDHGA8 BINDING SITE2 are HOST TARGET binding sites found in untranslated regions of mRNA encoded by PCDHGA8, corresponding to HOST TARGET binding sites such as BINDING SITE I, BINDING SITE II or BINDING SITE III. Table 2 illustrates the complementarity of the nucleotide sequences of PCDHGA8 BINDING SITE1 and PCDHGA8 BINDING SITE2, designated SEQ ID:2227 and SEQ ID: 1460 respectively, to the nucleotide sequence of VGAM233 RNA, herein designated VGAM RNA, also designated SEQ ID:568.

Another function of VGAM233 is therefore inhibition of Proto-cadherin Gamma Sub family A, 8 (PCDHGA8, Accession NM_{—}032088), a gene which is a potential calcium-dependent cell-adhesion protein. Accordingly, utilities of VGAM233 include diagnosis, prevention and treatment of diseases and clinical conditions associated with PCDHGA8. The function of PCDHGA8 and its association with various diseases and clinical conditions, has been established by previous studies, as described hereinabove with reference to VGAM207. SRY (sex determining region Y)-box 11 (SOX11, Accession NM_{—}003108) is another VGAM233 host target gene. SOX11 BINDING SITE is HOST TARGET binding site found in the 3′ untranslated region of mRNA encoded by SOX11, corresponding to a HOST TARGET binding site such as BINDING SITE I, BINDING SITE II or BINDING SITE III. Table 2 illustrates the complementarity of the nucleotide sequences of SOX11 BINDING SITE, designated SEQ ID:991, to the nucleotide sequence of VGAM233 RNA, herein designated VGAM RNA, also designated SEQ ID:568.

Another function of VGAM233 is therefore inhibition of SRY (sex determining region Y)-box 11 (SOX11, Accession NM_{—}003108). Accordingly, utilities of VGAM233 include diagnosis, prevention and treatment of diseases and clinical conditions associated with SOX11. FIG. 1 further provides a conceptual description of a novel bioinformatically detected viral gene of the present invention, referred to here as Viral Genomic Address Messenger 234 (VGAM234) viral gene, which modulates expression of respective host target genes thereof, the function and utility of which host target genes is known in the art.

VGAM234 is a novel bioinformatically detected regulatory, non protein coding, viral micro RNA (miRNA) gene. The method by which VGAM234 was detected is described hereinabove with reference to FIGS. 1-8.

VGAM234 gene, herein designated VGAM GENE, is a viral gene contained in the genome of Vaccinia Virus. VGAM234 host target gene, herein designated VGAM HOST TARGET GENE, is a human gene contained in the human genome.

VGAM234 gene encodes a VGAM234 precursor RNA, herein designated VGAM PRECURSOR RNA. Similar to other miRNA genes, and unlike most ordinary genes, VGAM234 precursor RNA does not encode a protein. A nucleotide sequence identical or highly similar to the nucleotide sequence of VGAM234 precursor RNA is designated SEQ ID:220, and is provided hereinbelow with reference to the sequence listing part. Nucleotide sequence SEQ ID:220 is located at position 123394 relative to the genome of Vaccinia Virus.

VGAM234 precursor RNA folds onto itself, forming VGAM234 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM234 folded precursor RNA into VGAM234 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM234 RNA is designated SEQ ID:569, and is provided hereinbelow with reference to the sequence listing part.

VGAM234 host target gene, herein designated VGAM HOST TARGET GENE, encodes a corresponding messenger RNA, VGAM234 host target RNA, herein designated VGAM HOST TARGET RNA. VGAM234 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM234 RNA, herein designated VGAM RNA, binds complementarily to one or more host target binding sites located in untranslated regions of VGAM234 host target RNA, herein designated VGAM HOST TARGET RNA. This complementary binding is due to the fact that the nucleotide sequence of VGAM234 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM234 RNA, herein designated VGAM RNA, may have a different number of host target binding sites in untranslated regions of a VGAM234 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM234 RNA, herein designated VGAM RNA, to host target binding sites on VGAM234 host target RNA, herein designated VGAM HOST TARGET RNA, such as BINDING SITE I, BINDING SITE II and BINDING SITE III, inhibits translation of VGAM234 host target RNA into VGAM234 host target protein, herein designated VGAM HOST TARGET PROTEIN. VGAM host target protein is therefore outlined by a broken line.

It is appreciated that VGAM234 host target gene, herein designated VGAM HOST TARGET GENE, in fact represents a plurality of VGAM234 host target genes. The mRNA of each one of this plurality of VGAM234 host target genes comprises one or more host target binding sites, each having a nucleotide sequence which is at least partly complementary to VGAM234 RNA, herein designated VGAM RNA, and which when bound by VGAM234 RNA causes inhibition of translation of respective one or more VGAM234 host target proteins.

It is further appreciated by one skilled in the art that the mode of translational inhibition illustrated by FIG. 1 with specific reference to translational inhibition exerted by VGAM234 gene, herein designated VGAM GENE, on one or more VGAM234 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM234 is inhibition of expression of host target genes, as part of a novel viral mechanism of attacking a host. Accordingly, utilities of VGAM234 include diagnosis, prevention and treatment of viral infection by Vaccinia Virus. Specific functions, and accordingly utilities, of VGAM234 correlate with, and may be deduced from, the identity of the host target genes which VGAM234 binds and inhibits, and the function of these host target genes, as elaborated hereinbelow.

Nucleotide sequences of the VGAM234 precursor RNA, herein designated VGAM PRECURSOR RNA, and of the ‘diced’ VGAM234 RNA, herein designated VGAM RNA, and a schematic representation of the secondary folding of VGAM234 folded precursor RNA, herein designated VGAM FOLDED PRECURSOR RNA, of VGAM234 are further described hereinbelow with reference to Table 1.

Nucleotide sequences of host target binding sites, such as BINDING SITE-I, BINDING SITE-II and BINDING SITE-III of FIG. 1, found on VGAM234 host target RNA, and schematic representation of the complementarity of each of these host target binding sites to VGAM234 RNA, herein designated VGAM RNA, are described hereinbelow with reference to Table 2.

As mentioned hereinabove with reference to FIG. 1, a function of VGAM234 gene, herein designated VGAM is inhibition of expression of VGAM234 target genes. It is appreciated that specific functions, and accordingly utilities, of VGAM234 correlate with, and may be deduced from, the identity of the target genes which VGAM234 binds and inhibits, and the function of these target genes, as elaborated hereinbelow.

Complement Component 6 (C6, Accession XM_{—}170508) is a VGAM234 host target gene. C6 BINDING SITE is HOST TARGET binding site found in the 5′ untranslated region of mRNA encoded by C6, corresponding to a HOST TARGET binding site such as BINDING SITE I, BINDING SITE II or BINDING SITE III. Table 2 illustrates the complementarity of the nucleotide sequences of C6 BINDING SITE, designated SEQ ID:3656, to the nucleotide sequence of VGAM234 RNA, herein designated VGAM RNA, also designated SEQ ID:569.

A function of VGAM234 is therefore inhibition of Complement Component 6 (C6, Accession XM_{—}170508). Accordingly, utilities of VGAM234 include diagnosis, prevention and treatment of diseases and clinical conditions associated with C6. Chorionic Gonadotropin, Beta Polypeptide (CGB, Accession NM_{—}000737) is another VGAM234 host target gene. CGB BINDING SITE is HOST TARGET binding site found in the 5′ untranslated region of mRNA encoded by CGB, corresponding to a HOST TARGET binding site such as BINDING SITE I, BINDING SITE II or BINDING SITE III. Table 2 illustrates the complementarity of the nucleotide sequences of CGB BINDING SITE, designated SEQ ID:770, to the nucleotide sequence of VGAM234 RNA, herein designated VGAM RNA, also designated SEQ ID:569.

Another function of VGAM234 is therefore inhibition of Chorionic Gonadotropin, Beta Polypeptide (CGB, Accession NM_{—}000737), a gene which stimulates the ovaries to synthesize the steroids. Accordingly, utilities of VGAM234 include diagnosis, prevention and treatment of diseases and clinical conditions associated with CGB. The function of CGB has been established by previous studies. Human chorionic gonadotropin (hCG) is a glycoprotein hormone produced by trophoblastic cells of the placenta beginning 10 to 12 days after conception. Maintenance of the fetus in the first trimester of pregnancy requires the production of hCG, which binds to the corpus luteum of the ovary which is stimulated to produce progesterone which in turn maintains the secretory endometrium. See 118850. Boorstein et al. (1982) concluded that the beta subunit of CG is encoded by at least 8 genes arranged in tandem and inverted pairs. They stated that ‘until sequence analysis is complete, we cannot exclude the possibility that the eight genes include some pseudogenes or the related gene, beta-LH.’ The beta subunits of luteinizing hormone (LHB) and CG show about 82% amino acid homology. The homology with beta-FSH and beta-TSH is much lower. Policastro et al. (1983, 1986) found 6 nonallelic copies of the CGB gene and a single-copy LHB gene. All were contained in a single 58-kb EcoRI fragment. The hCG beta-subunit is unique in the family of beta-containing glycoprotein hormones in that it contains an extension of 29 amino acids at its COOH end. Amato et al. (2002) reported a patient with a 9-year history of secondary infertility due to an anti-CG autoantibody. Although she had regular menstrual cycles, had conceived spontaneously, and had good hormonal and follicular responses to gonadotropic stimulation regimens during the in vitro fertilization workup, she presented with apparent recurrent pregnancy loss associated with prolonged raised CG levels. She was found to have specific, low-affinity, but high-capacity anti-CG antibody. Crossreaction with recombinant FSH, recombinant LH, CG-alpha, and CG-beta was low. In addition, heat-inactivated serum and the affinity-purified IgG were shown to inhibit the action of CG in an in vitro bioassay. The authors concluded that the persisting titer of the antibody was responsible for the patient's infertility.

Full details of the abovementioned studies are described in the following publications, the disclosure of which are hereby incorporated by reference:
Policastro, P. F.; Daniels-McQueen, S.; Carle, G.; Boime, I.: A map of the hCG-beta-LH-beta gene cluster. J. Biol. Chem. 261: 5907-5916, 1986.; and
Amato, F.; Warnes, G. M.; Kirby, C. A.; Norman, R. J.: Infertility caused by hCG autoantibody. J. Clin. Endocr. Metab. 87: 993-997, 2002.

Further studies establishing the function and utilities of CGB are found in John Hopkins OMIM database record ID 118860, and in sited publications numbered 2862, 2924-2929, 2492, 286 and 2930-2933 listed in the bibliography section hereinbelow, which are also hereby incorporated by reference. Cullin 3 (CUL3, Accession NM_{—}003590) is another VGAM234 host target gene. CUL3 BINDING SITE is HOST TARGET binding site found in the 3′ untranslated region of mRNA encoded by CUL3, corresponding to a HOST TARGET binding site such as BINDING SITE I, BINDING SITE II or BINDING SITE III. Table 2 illustrates the complementarity of the nucleotide sequences of CUL3 BINDING SITE, designated SEQ ID: 1040, to the nucleotide sequence of VGAM234 RNA, herein designated VGAM RNA, also designated SEQ ID:569.

Another function of VGAM234 is therefore inhibition of Cullin 3 (CUL3, Accession NM_{—}003590), a gene which may target other proteins for ubiquitin-dependent proteolysis. Accordingly, utilities of VGAM234 include diagnosis, prevention and treatment of diseases and clinical conditions associated with CUL3. The function of CUL3 has been established by previous studies. Kipreos et al. (1996) identified a conserved gene family, designated cullins, with at least 5 members in nematodes, 6 in humans, and 3 in S. cerevisiae. See CULL (OMIM Ref. No. 603134). Human CUL3 is an ortholog of nematode cul3. Michel and Xiong (1998) identified human CUL3 cDNAs and reported that the predicted protein is 768 amino acids long. Ishikawa et al. (1998) isolated a CUL3 cDNA, KIAA0617, as 1 of 100 brain cDNAs encoding large proteins. Using RT-PCR, they found that CUL3 is expressed in several tissues. Du et al. (1998) identified CUL3 as a gene whose expression in human fibroblasts is induced by phorbol 12-myristate 13-acetate (PMA) and suppressed by salicylate. They reported that the sequences of the human and C. elegans cul3 proteins share 46% identity. Northern blot analysis revealed that CUL3 is expressed as major 2.8- and minor 4.3-kb transcripts in various human tissues, with the highest levels in skeletal muscle and heart.

Full details of the abovementioned studies are described in the following publications, the disclosure of which are hereby incorporated by reference:
Du, M.; Sansores-Garcia, L.; Zu, Z.; Wu, K. K.: Cloning and expression analysis of a novel salicylate suppressible gene, Hs-CUL-3, a member of cullin/Cdc53 family. J. Biol. Chem. 273: 24289-24292, 1998.; and
Ishikawa, K.; Nagase, T.; Suyama, M.; Miyajima, N.; Tanaka, A.; Kotani, H.; Nomura, N.; Ohara, O.: Prediction of the coding sequences of unidentified human genes. X. The complete seque.

Further studies establishing the function and utilities of CUL3 are found in John Hopkins OMIM database record ID 603136, and in sited publications numbered 1151, 2136-213 and 1150 listed in the bibliography section hereinbelow, which are also hereby incorporated by reference. Glutamate Receptor, Ionotropic, N-methyl Daspartate-like 1A (GRINL1A, Accession XM_{—}045376) is another VGAM234 host target gene. GRINL1A BINDING SITE is HOST TARGET binding site found in the 3′ untranslated region of mRNA encoded by GRINL1A, corresponding to a HOST TARGET binding site such as BINDING SITE I, BINDING SITE II or BINDING SITE III. Table 2 illustrates the complementarity of the nucleotide sequences of GRINL1A BINDING SITE, designated SEQ ID:2863, to the nucleotide sequence of VGAM234 RNA, herein designated VGAM RNA, also designated SEQ ID:569.

Another function of VGAM234 is therefore inhibition of Glutamate Receptor, Ionotropic, N-methyl D-aspartate-like 1A (GRINL1A, Accession XM_{—}045376), a gene which plays a role in the development and function of the mammalian brain. Accordingly, utilities of VGAM234 include diagnosis, prevention and treatment of diseases and clinical conditions associated with GRINL1A. The function of GRINL1A has been established by previous studies. The ionotropic glutamate receptors (e.g., GRIN1; 138249) play roles in the development and function of the mammalian brain. Roginski et al. (2001) isolated rat GRINL1 cDNA from a size-fractionated, directional rat brain cDNA library. They obtained human GRINL1A genomic DNA sequences by amplification with primers designed from the rat sequences. By fluorescence in situ hybridization, Roginski et al. (2001) mapped the GRINL1A gene to chromosome 15q22.1. By interspecific backcross analysis, Wydner et al. (2001) mapped the mouse Grinl1a gene to chromosome 9 in a region that shares linkage conservation with human chromosome 15q21-q22.

Full details of the abovementioned studies are described in the following publications, the disclosure of which are hereby incorporated by reference:
Roginski, R. S.; Mohan Raj, B. K.; Finkernagel, S. W.; Sciorra, L. J.: Assignment of an ionotropic glutamate receptor-like gene (GRINL1A) to human chromosome 15q22.1 by in situ hybridization. Cytogenet. Cell Genet. 93: 143-144, 2001.; and
Wydner, K. S.; Mohan Raj, B. K.; Sciorra, L. J.; Roginski, R. S.: The mouse orthologue of the human ionotropic glutamate receptor-like gene (GRINL1A) maps to mouse chromosome 9. Cytog.

Further studies establishing the function and utilities of GRINL1A are found in John Hopkins OMIM database record ID 606485, and in sited publications numbered 1273-1274 listed in the bibliography section hereinbelow, which are also hereby incorporated by reference. Protein Kinase, CGMP-dependent, Type I (PRKG1, Accession NM_{—}006258) is another VGAM234 host target gene. PRKG1 BINDING SITE is HOST TARGET binding site found in the 3′ untranslated region of mRNA encoded by PRKG1, corresponding to a HOST TARGET binding site such as BINDING SITE I, BINDING SITE II or BINDING SITE III. Table 2 illustrates the complementarity of the nucleotide sequences of PRKG1 BINDING SITE, designated SEQ ID:1283, to the nucleotide sequence of VGAM234 RNA, herein designated VGAM RNA, also designated SEQ ID:569.

Another function of VGAM234 is therefore inhibition of Protein Kinase, CGMP-dependent, Type I (PRKG1, Accession NM_{—}006258), a gene which relaxes vascular smooth muscle and inhibits platelet aggregation. Accordingly, utilities of VGAM234 include diagnosis, prevention and treatment of diseases and clinical conditions associated with PRKG1. The function of PRKG1 has been established by previous studies. Cyclic GMP and cyclic GMP-dependent protein kinase play important roles in physiologic processes such as relaxation of vascular smooth muscle and inhibition of platelet aggregation. Two main forms of cGK have been identified: a soluble form designated type I and an intrinsic membrane-bound form designated type II. Sandberg et al. (1989) isolated and characterized cDNA clones for the type I beta isozyme from human placenta libraries. The same group used a genomic probe for mapping the gene (Orstavik et al., 1992). By Southern blots of human/hamster somatic cell hybrids, they localized the PRKGR1B gene to chromosome 10. The gene was regionally localized to 10q11.2 by in situ hybridization. Tamura et al. (1996) cloned a human cGKI-alpha cDNA by RT-PCR of aorta RNA using primers based on the sequence of a bovine cGKI-alpha cDNA. The predicted 671-amino acid human cGKI-alpha protein is nearly identical to bovine cGKI-alpha. Based on Southern blot and sequence analyses, Tamura et al. (1996) suggested that cGKI-alpha and cGKI-beta are generated by alternative splicing of a single gene that maps to chromosome 10. By Northern blot analysis, cGKI-alpha was abundantly expressed as a 7.0-kb mRNA in aorta, heart, kidneys and adrenals; the 7.0-kb cGKI-beta mRNA was abundantly expressed only in the uterus. Orstavik et al. (1997) noted that type I cGK is a homodimer, with each monomer containing a regulatory cGMP-binding domain and a catalytic domain. They reported that the type I cGK gene consists of 19 exons spanning at least 220 kb. The first 2 exons, which the authors called 1-alpha and 1-beta, are used alternatively and encode the alpha isoform- and beta isoform-specific sequences. By Northern blot analysis, type I cGK-alpha mRNA was most abundant in lung and placenta, while type I cGK-beta was expressed at highest levels in bladder, uterus, adrenal gland, and fallopian tube. Orstavik et al. (1997) noted that 5 of the 7 splice sites in the Drosophila melanogaster DG2 gene, which encodes a cGK, are also present in the human type I cGK gene. Osborne et al. (1997) reported that levels of the DG2-encoded cGK in Drosophila affect food-search behavior and account for a naturally occurring behavioral polymorphism. Animal model experiments lend further support to the function of PRKG1. Pfeifer et al. (1998) generated mice deficient in cGKI by targeted disruption. Loss of cGKI abolished nitric oxide/cGMP-dependent relaxation of smooth muscle, resulting in severe vascular and intestinal dysfunction. However, cGKI-deficient smooth muscle responded normally to cAMP, indicating that cAMP and cGMP signal via independent pathways, with cGKI being the specific mediator of the nitric oxide/cGMP effects in murine smooth muscle.

It is appreciated that the abovementioned animal model for PRKG1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Pfeifer, A.; Klatt, P.; Massberg, S.; Ny, L.; Sausbier, M.; Hirneill, C.; Wang, G.-X.; Korth, M.; Aszodi, A.; Andersson, K.-E.; Krombach, F.; Mayerhofer, A.; Ruth, P.; Fassler, R.; Hofmann, F.: Defective smooth muscle regulation in cGMP kinase 1-deficient mice. EMBO J. 17: 3045-3051, 1998.; and
Sandberg, M.; Natarajan, V.; Ronander, I.; Kalderon, D.; Walter, U.; Lohmann, S. M.; Jahnsen, T.: Molecular cloning and predicted full-length amino acid sequence of the type I beta isoz.

Further studies establishing the function and utilities of PRKG1 are found in John Hopkins OMIM database record ID 176894, and in sited publications numbered 273-278 listed in the bibliography section hereinbelow, which are also hereby incorporated by reference. Wolf-Hirschhorn Syndrome Candidate 1 (WHSC1, Accession NM_{—}133334) is another VGAM234 host target gene. WHSC1 BINDING SITE is HOST TARGET binding site found in the 3′ untranslated region of mRNA encoded by WHSC1, corresponding to a HOST TARGET binding site such as BINDING SITE I, BINDING SITE II or BINDING SITE III. Table 2 illustrates the complementarity of the nucleotide sequences of WHSC1 BINDING SITE, designated SEQ ID:2409, to the nucleotide sequence of VGAM234 RNA, herein designated VGAM RNA, also designated SEQ ID:569.

Another function of VGAM234 is therefore inhibition of Wolf-Hirschhorn Syndrome Candidate 1 (WHSC1, Accession NM_{—}133334), a gene which binds covalently to and repairs g/t mismatches. Accordingly, utilities of VGAM234 include diagnosis, prevention and treatment of diseases and clinical conditions associated with WHSC1. The function of WHSC1 has been established by previous studies. Wolf-Hirschhorn syndrome (WHS; 194190) is a malformation syndrome associated with a hemizygous deletion of the distal short arm of chromosome 4 (OMIM Ref. No. 4p16.3). The shortest region of overlap of the deletions observed in WHS patients, the WHS critical region, has been confined to a region of 165 kb (Wright et al., 1997). This region was sequenced completely during the search for the Huntington disease gene (Baxendale et al., 1993). Stec et al. (1998) described a novel developmental gene, two-thirds of which maps in the distal part of the WHS critical region. They designated the gene WHSC1 (Wolf-Hirschhorn syndrome candidate-1). The WHSC1 gene was identified initially through its high similarity to the translation product of an expressed sequence tag, located in the 165-kb WHCR, with the SET domain (see OMIM Ref. No. 600960) of the Drosophila protein ASH1 (OMIM Ref. No. 100790). The SET domain is found in proteins that are involved in embryonic development. The 25-exon WHSC1 gene was found to be expressed ubiquitously in early development and to undergo complex alternative splicing and differential polyadenylation. It encodes a 136-kD protein containing 4 domains present in other developmental proteins: a PWWP domain, an HMG box, a SET domain also found in the Drosophila dysmorphy gene ash-encoded protein, and a PHD-type zinc finger. It is expressed preferentially in rapidly growing embryonic tissues, in a pattern corresponding to affected or gans in WHS patients. The nature of the protein motifs, the expression pattern, and its mapping to the critical region led Stec et al. (1998) to propose WHSC1 as a good candidate gene to be responsible for many of the phenotypic features of WHS. Stec et al. (1998) noted that the t(4;14)(p16.3;q32.3) translocations described in a significant fraction of multiple myelomas (Richelda et al., 1997; Chesi et al., 1997) have breakpoints located less than 100 kb centromeric of the FGFR3 gene (OMIM Ref. No. 134934) on 4p16.3. They found that at least 3 of the breakpoints merged the immunoglobulin heavy-chain gene (IGHG1; 147100) on chromosome 14 with the WHSC1 gene. This fusion of genes and their untimely expression in the myeloid lineage driven from the 5-prime IgH enhancer may indicate that WHSC1-encoded proteins are involved in the clinical heterogeneity of multiple myeloma.

Full details of the abovementioned studies are described in the following publications, the disclosure of which are hereby incorporated by reference:
Chesi, M.; Nardini, E.; Brents, L. A.; Schrock, E.; Ried, T.; Kuehl, W. M.; Bergsagel, P. L.: Frequent translocation t(4;14)(p16.3;q32.3) in multiple myeloma is associated with increased expression and activating mutations of fibroblast growth factor receptor 3. Nature Genet. 16: 260-264, 1997.; and
Richelda, R.; Ronchetti, D.; Baldini, L.; Cro, L.; Viggiano, L.; Marzella, R.; Rocchi, M.; Otsuki, T.; Lombardi, L.; Maiolo, A. T.; Neri, A.: A novel chromosomal translocation t(4;14)(p16.

Further studies establishing the function and utilities of WHSC1 are found in John Hopkins OMIM database record ID 602952, and in sited publications numbered 236, 263 and 1823 listed in the bibliography section hereinbelow, which are also hereby incorporated by reference. Chorionic Gonadotropin, Beta Polypeptide 5 (CGB5, Accession NM_{—}033043) is another VGAM234 host target gene. CGB5 BINDING SITE is HOST TARGET binding site found in the 5′ untranslated region of mRNA encoded by CGB5, corresponding to a HOST TARGET binding site such as BINDING SITE I, BINDING SITE II or BINDING SITE III. Table 2 illustrates the complementarity of the nucleotide sequences of CGB5 BINDING SITE, designated SEQ ID:2311, to the nucleotide sequence of VGAM234 RNA, herein designated VGAM RNA, also designated SEQ ID:569.

Another function of VGAM234 is therefore inhibition of Chorionic Gonadotropin, Beta Polypeptide 5 (CGB5, Accession NM_{—}033043). Accordingly, utilities of VGAM234 include diagnosis, prevention and treatment of diseases and clinical conditions associated with CGB5. DEAD/H (Asp-Glu-Ala-Asp/His) Box Polypeptide 33 (DDX33, Accession NM_{—}020162) is another VGAM234 host target gene. DDX33 BINDING SITE is HOST TARGET binding site found in the 3′ untranslated region of mRNA encoded by DDX33, corresponding to a HOST TARGET binding site such as BINDING SITE I, BINDING SITE II or BINDING SITE III. Table 2 illustrates the complementarity of the nucleotide sequences of DDX33 BINDING SITE, designated SEQ ID:1895, to the nucleotide sequence of VGAM234 RNA, herein designated VGAM RNA, also designated SEQ ID:569.

Another function of VGAM234 is therefore inhibition of DEAD/H (Asp-Glu-Ala-Asp/His) Box Polypeptide 33 (DDX33, Accession NM_{—}020162). Accordingly, utilities of VGAM234 include diagnosis, prevention and treatment of diseases and clinical conditions associated with DDX33. FLJ20296 (Accession NM_{—}017750) is another VGAM234 host target gene. FLJ20296 BINDING SITE is HOST TARGET binding site found in the 5′ untranslated region of mRNA encoded by FLJ20296, corresponding to a HOST TARGET binding site such as BINDING SITE I, BINDING SITE II or BINDING SITE III. Table 2 illustrates the complementarity of the nucleotide sequences of FLJ20296 BINDING SITE, designated SEQ ID:1749, to the nucleotide sequence of VGAM234 RNA, herein designated VGAM RNA, also designated SEQ ID:569.

Another function of VGAM234 is therefore inhibition of FLJ20296 (Accession NM_{—}017750). Accordingly, utilities of VGAM234 include diagnosis, prevention and treatment of diseases and clinical conditions associated with FLJ20296. IRO039700 (Accession NM_{—}018671) is another VGAM234 host target gene. IRO039700 BINDING SITE is HOST TARGET binding site found in the 5′ untranslated region of mRNA encoded by IRO039700, corresponding to a HOST TARGET binding site such as BINDING SITE I, BINDING SITE II or BINDING SITE III. Table 2 illustrates the complementarity of the nucleotide sequences of IRO039700 BINDING SITE, designated SEQ ID:1858, to the nucleotide sequence of VGAM234 RNA, herein designated VGAM RNA, also designated SEQ ID:569.

Another function of VGAM234 is therefore inhibition of IRO039700 (Accession NM_{—}018671). Accordingly, utilities of VGAM234 include diagnosis, prevention and treatment of diseases and clinical conditions associated with IRO039700. KIAA0594 (Accession XM_{—}036117) is another VGAM234 host target gene. KIAA0594 BINDING SITE is HOST TARGET binding site found in the 3′ untranslated region of mRNA encoded by KIAA0594, corresponding to a HOST TARGET binding site such as BINDING SITE I, BINDING SITE II or BINDING SITE III. Table 2 illustrates the complementarity of the nucleotide sequences of KIAA0594 BINDING SITE, designated SEQ ID:2706, to the nucleotide sequence of VGAM234 RNA, herein designated VGAM RNA, also designated SEQ ID:569.

Another function of VGAM234 is therefore inhibition of KIAA0594 (Accession XM_{—}036117). Accordingly, utilities of VGAM234 include diagnosis, prevention and treatment of diseases and clinical conditions associated with KIAA0594. Mal, T-cell Differentiation Protein 2 (MAL2, Accession NM_{—}052886) is another VGAM234 host target gene. MAL2 BINDING SITE is HOST TARGET binding site found in the 3′ untranslated region of mRNA encoded by MAL2, corresponding to a HOST TARGET binding site such as BINDING SITE I, BINDING SITE II or BINDING SITE III. Table 2 illustrates the complementarity of the nucleotide sequences of MAL2 BINDING SITE, designated SEQ ID:2343, to the nucleotide sequence of VGAM234 RNA, herein designated VGAM RNA, also designated SEQ ID:569.

Another function of VGAM234 is therefore inhibition of Mal, T-cell Differentiation Protein 2 (MAL2, Accession NM_{—}052886). Accordingly, utilities of VGAM234 include diagnosis, prevention and treatment of diseases and clinical conditions associated with MAL2. LOC145622 (Accession XM_{—}085186) is another VGAM234 host target gene. LOC145622 BINDING SITE is HOST TARGET binding site found in the 3′ untranslated region of mRNA encoded by LOC145622, corresponding to a HOST TARGET binding site such as BINDING SITE I, BINDING SITE II or BINDING SITE III. Table 2 illustrates the complementarity of the nucleotide sequences of LOC145622 BINDING SITE, designated SEQ ID:3080, to the nucleotide sequence of VGAM234 RNA, herein designated VGAM RNA, also designated SEQ ID:569.

Another function of VGAM234 is therefore inhibition of LOC145622 (Accession XM_{—}085186). Accordingly, utilities of VGAM234 include diagnosis, prevention and treatment of diseases and clinical conditions associated with LOC145622. LOC152982 (Accession XM_{—}087574) is another VGAM234 host target gene. LOC152982 BINDING SITE is HOST TARGET binding site found in the 3′ untranslated region of mRNA encoded by LOC152982, corresponding to a HOST TARGET binding site such as BINDING SITE I, BINDING SITE II or BINDING SITE III. Table 2 illustrates the complementarity of the nucleotide sequences of LOC152982 BINDING SITE, designated SEQ ID:3176, to the nucleotide sequence of VGAM234 RNA, herein designated VGAM RNA, also designated SEQ ID:569.

Another function of VGAM234 is therefore inhibition of LOC152982 (Accession XM_{—}087574). Accordingly, utilities of VGAM234 include diagnosis, prevention and treatment of diseases and clinical conditions associated with LOC152982. LOC256174 (Accession XM_{—}174823) is another VGAM234 host target gene. LOC256174 BINDING SITE is HOST TARGET binding site found in the 5′ untranslated region of mRNA encoded by LOC256174, corresponding to a HOST TARGET binding site such as BINDING SITE I, BINDING SITE II or BINDING SITE III. Table 2 illustrates the complementarity of the nucleotide sequences of LOC256174 BINDING SITE, designated SEQ ID:3742, to the nucleotide sequence of VGAM234 RNA, herein designated VGAM RNA, also designated SEQ ID:569.

Another function of VGAM234 is therefore inhibition of LOC256174 (Accession XM_{—}174823). Accordingly, utilities of VGAM234 include diagnosis, prevention and treatment of diseases and clinical conditions associated with LOC256174. FIG. 1 further provides a conceptual description of a novel bioinformatically detected viral gene of the present invention, referred to here as Viral Genomic Address Messenger 235 (VGAM235) viral gene, which modulates expression of respective host target genes thereof, the function and utility of which host target genes is known in the art.

VGAM235 is a novel bioinformatically detected regulatory, non protein coding, viral micro RNA (miRNA) gene. The method by which VGAM235 was detected is described hereinabove with reference to FIGS. 1-8.

VGAM235 gene, herein designated VGAM GENE, is a viral gene contained in the genome of Vaccinia Virus. VGAM235 host target gene, herein designated VGAM HOST TARGET GENE, is a human gene contained in the human genome.

VGAM235 gene encodes a VGAM235 precursor RNA, herein designated VGAM PRECURSOR RNA. Similar to other miRNA genes, and unlike most ordinary genes, VGAM235 precursor RNA does not encode a protein. A nucleotide sequence identical or highly similar to the nucleotide sequence of VGAM235 precursor RNA is designated SEQ ID:221, and is provided hereinbelow with reference to the sequence listing part. Nucleotide sequence SEQ ID:221 is located at position 123220 relative to the genome of Vaccinia Virus.

VGAM235 precursor RNA folds onto itself, forming VGAM235 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM235 folded precursor RNA into VGAM235 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM235 RNA is designated SEQ ID:570, and is provided hereinbelow with reference to the sequence listing part.

VGAM235 host target gene, herein designated VGAM HOST TARGET GENE, encodes a corresponding messenger RNA, VGAM235 host target RNA, herein designated VGAM HOST TARGET RNA. VGAM235 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM235 RNA, herein designated VGAM RNA, binds complementarily to one or more host target binding sites located in untranslated regions of VGAM235 host target RNA, herein designated VGAM HOST TARGET RNA. This complementary binding is due to the fact that the nucleotide sequence of VGAM235 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM235 RNA, herein designated VGAM RNA, may have a different number of host target binding sites in untranslated regions of a VGAM235 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM235 RNA, herein designated VGAM RNA, to host target binding sites on VGAM235 host target RNA, herein designated VGAM HOST TARGET RNA, such as BINDING SITE I, BINDING SITE II and BINDING SITE III, inhibits translation of VGAM235 host target RNA into VGAM235 host target protein, herein designated VGAM HOST TARGET PROTEIN. VGAM host target protein is therefore outlined by a broken line.

It is appreciated that VGAM235 host target gene, herein designated VGAM HOST TARGET GENE, in fact represents a plurality of VGAM235 host target genes. The mRNA of each one of this plurality of VGAM235 host target genes comprises one or more host target binding sites, each having a nucleotide sequence which is at least partly complementary to VGAM235 RNA, herein designated VGAM RNA, and which when bound by VGAM235 RNA causes inhibition of translation of respective one or more VGAM235 host target proteins.

It is further appreciated by one skilled in the art that the mode of translational inhibition illustrated by FIG. 1 with specific reference to translational inhibition exerted by VGAM235 gene, herein designated VGAM GENE, on one or more VGAM235 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM235 is inhibition of expression of host target genes, as part of a novel viral mechanism of attacking a host. Accordingly, utilities of VGAM235 include diagnosis, prevention and treatment of viral infection by Vaccinia Virus. Specific functions, and accordingly utilities, of VGAM235 correlate with, and may be deduced from, the identity of the host target genes which VGAM235 binds and inhibits, and the function of these host target genes, as elaborated hereinbelow.

Nucleotide sequences of the VGAM235 precursor RNA, herein designated VGAM PRECURSOR RNA, and of the ‘diced’ VGAM235 RNA, herein designated VGAM RNA, and a schematic representation of the secondary folding of VGAM235 folded precursor RNA, herein designated VGAM FOLDED PRECURSOR RNA, of VGAM235 are further described hereinbelow with reference to Table 1.

Nucleotide sequences of host target binding sites, such as BINDING SITE-I, BINDING SITE-II and BINDING SITE-III of FIG. 1, found on VGAM235 host target RNA, and schematic representation of the complementarity of each of these host target binding sites to VGAM235 RNA, herein designated VGAM RNA, are described hereinbelow with reference to Table 2.

As mentioned hereinabove with reference to FIG. 1, a function of VGAM235 gene, herein designated VGAM is inhibition of expression of VGAM235 target genes. It is appreciated that specific functions, and accordingly utilities, of VGAM235 correlate with, and may be deduced from, the identity of the target genes which VGAM235 binds and inhibits, and the function of these target genes, as elaborated hereinbelow.

Calcitonin Receptor (CALCR, Accession NM_{—}001742) is a VGAM235 host target gene. CALCR BINDING SITE is HOST TARGET binding site found in the 3′ untranslated region of mRNA encoded by CALCR, corresponding to a HOST TARGET binding site such as BINDING SITE I, BINDING SITE II or BINDING SITE III. Table 2 illustrates the complementarity of the nucleotide sequences of CALCR BINDING SITE, designated SEQ ID:854, to the nucleotide sequence of VGAM235 RNA, herein designated VGAM RNA, also designated SEQ ID:570.

A function of VGAM235 is therefore inhibition of Calcitonin Receptor (CALCR, Accession NM_{—}001742), a gene which is a receptor for calcitonin, is mediated by g proteins which activate adenylyl cyclase, and thought to couple to the heterotrimeric guanosine triphosphate-binding protein that is sensitive to cholera toxin. Accordingly, utilities of VGAM235 include diagnosis, prevention and treatment of diseases and clinical conditions associated with CALCR. The function of CALCR has been established by previous studies. Taboulet et al. (1996) had reported the point mutation polymorphism (OMIM Ref. No. T to C) in the 3-prime region of the CALCR gene which induced a pro447-to-leu amino acid change in the third intracellular domain of the protein. This was the same mutation as that subsequently identified by Nakamura et al. (1997) and Masi et al. (1998) and referred to as pro463 to leu; the difference in numbering depended on whether isoform 1 or isoform 2 of the calcitonin receptor, with or without the 16-amino acid insert, was referred to (de Vernejoul, 1999). Taboulet et al. (1998) studied the distribution of these alleles in a cohort of 123 women with no osteoporotic fractures and 92 women who presented with one or more osteoporotic fractures of wrist or vertebrae. They found that bone mineral density of the femoral neck was significantly higher in heterozygous subjects compared with the homozygous leucine and homozygous proline genotypes. Also, a decreased fracture risk was observed in heterozygote subjects. In conclusion, they suggested that polymorphism of CALCR is associated with osteoporotic factors and bone mineral density in a population of postmenopausal women. The heterozygous advantage of the pro/leu subjects could explain their protection against osteoporosis. The distribution of the CALCR alleles in the French population studied by Taboulet et al. (1998) was quite different from that observed by Nakamura et al. (1997) in the Japanese population. In Japan, the proline homozygote was the most frequent genotype (70%), Gorn et al. (1992) cloned a human calcitonin receptor cDNA from a eukaryotic expression library prepared from an ovarian small cell carcinoma cell line. A cell line had been shown to respond to calcitonin (CT, or CALCA; 114130) with increases in content of cellular cAMP. Transfection of this cDNA into COS cells resulted in expression of receptors with high affinity for salmon and human calcitonin. The expressed CALCR was coupled to adenylate cyclase. Northern analysis indicated a single transcript of about 4.2 kb. The cloned cDNA encoded a putative peptide of 490 amino acids with 7 potential transmembrane domains. The amino acid sequence was 73% identical to porcine CALCR, although the human CALCR contained an inset of 16 amino acids between transmembrane domains I and II. CALCR is closely related to the parathyroid hormone receptor (OMIM Ref. No. 168468) and the secretin receptor (OMIM Ref. No. 182098); these receptors comprise a distinct family of G protein-coupled 7-transmembrane domain receptors. A comparison of the human CALCR sequence to protein sequences in databases suggested that the receptor for calcitonin is evolutionarily related to the chemoattractant receptor of the primitive eukaryote Dictyostelium discoideum.

Full details of the abovementioned studies are described in the following publications, the disclosure of which are hereby incorporated by reference:
Gorn, A. H.; Lin, H. Y.; Yamin, M.; Auron, P. E.; Flannery, M. R.; Tapp, D. R.; Manning, C. A.; Lodish, H. F.; Krane, S. M.; Goldring, S. R.: Cloning, characterization, and expression of a human calcitonin receptor from an ovarian carcinoma cell line. J. Clin. Invest. 90: 1726-1735, 1992.; and
Taboulet, J.; Frenkian, M.; Frendo, J. L.; Feingold, N.; Jullienne, A.; de Vernejoul, M. C.: Calcitonin receptor polymorphism is associated with a decreased fracture risk in post-menop.

Further studies establishing the function and utilities of CALCR are found in John Hopkins OMIM database record ID 114131, and in sited publications numbered 542-544, and 3028-3033 listed in the bibliography section hereinbelow, which are also hereby incorporated by reference. Interleukin 1 Family, Member 9 (IL1F9, Accession NM_{—}019618) is another VGAM235 host target gene. IL1F9 BINDING SITE is HOST TARGET binding site found in the 3′ untranslated region of mRNA encoded by IL1F9, corresponding to a HOST TARGET binding site such as BINDING SITE I, BINDING SITE II or BINDING SITE III. Table 2 illustrates the complementarity of the nucleotide sequences of IL1F9 BINDING SITE, designated SEQ ID:1886, to the nucleotide sequence of VGAM235 RNA, herein designated VGAM RNA, also designated SEQ ID:570.

Another function of VGAM235 is therefore inhibition of Interleukin 1 Family, Member 9 (IL1F9, Accession NM_{—}019618), a gene which initiates and promotes the host response to injury or infection by activating a set of transcription factors. Accordingly, utilities of VGAM235 include diagnosis, prevention and treatment of diseases and clinical conditions associated with IL1F9. The function of IL1F9 has been established by previous studies. The cytokine interleukin-1 (IL1; OMIM Ref. No. 147760) elicits a wide array of biologic activities that initiate and promote the host response to injury or infection by activating a set of transcription factors, including NFKB (see OMIM Ref. No. 164011) and AP1 (see OMIM Ref. No. 165160), which in turn induce production of effectors of the inflammatory response. By searching EST databases for IL1 homologs, Kumar et al. (2000) identified a cDNA encoding IL1H1. The deduced 169-amino acid protein, which shares 19 to 32% sequence similarity with other IL1 family members, lacks a hydrophobic leader peptide and a prodomain. Biophysical analyses indicated that IL1H1 has 12 beta strands, similar to other IL1 family members. Northern blot analysis detected no expression of IL1H1 in a multiple tissue blot. PCR analysis detected low levels of expression in lungs and macrophages. RT-PCR and Western blot analyses showed that cytokine-induced keratinocytes expressed readily detectable IL1H1. In situ hybridization analysis demonstrated IL1H1 expression in keratinocytes in a mouse model of contact hypersensitivity or herpes simplex virus infection. By sequencing a BAC clone containing the IL1B gene (OMIM Ref. No. 147720) and searching EST databases for IL1-like ligands, Busfield et al. (2000) identified IL1H1, which they designated IL1RP2. SDS-PAGE analysis showed that IL1H1 is expressed as a 20-kD protein. Northern blot analysis detected a 1.8-kb IL1H1 transcript in the squamous cell epithelium of the esophagus. In situ hybridization analysis revealed expression in the middle layers of epithelium. In vitro, IL1H1 message was rapidly induced following stimulation of keratinocytes. Reporter and bioassays failed to detect agonist or antagonist activity for NFKB.

Full details of the abovementioned studies are described in the following publications, the disclosure of which are hereby incorporated by reference:
Busfield, S. J.; Comrack, C. A.; Yu, G.; Chickering, T. W.; Smutko, J. S.; Zhou, H.; Leiby, K. R.; Holmgren, L. M.; Gearing, D. P.; Pan, Y.: Identification and gene organization of three novel members of the IL-1 family on human chromosome 2. Genomics 66: 213-216, 2000.; and
Kumar, S.; McDonnell, P. C.; Lehr, R.; Tierney, L.; Tzimas, M. N.; Griswold, D. E.; Capper, E. A.; Tal-Singer, R.; Wells, G. I.; Doyle, M. L.; Young, P. R.: Identification and initial.

Further studies establishing the function and utilities of IL1F9 are found in John Hopkins OMIM database record ID 605542, and in sited publications numbered 1007 listed in the bibliography section hereinbelow, which are also hereby incorporated by reference. Lymphotoxin Alpha (TNF super family, member 1) (LTA, Accession NM_{—}000595) is another VGAM235 host target gene. LTA BINDING SITE is HOST TARGET binding site found in the 3′ untranslated region of mRNA encoded by LTA, corresponding to a HOST TARGET binding site such as BINDING SITE I, BINDING SITE II or BINDING SITE III. Table 2 illustrates the complementarity of the nucleotide sequences of LTA BINDING SITE, designated SEQ ID:757, to the nucleotide sequence of VGAM235 RNA, herein designated VGAM RNA, also designated SEQ ID:570.

Another function of VGAM235 is therefore inhibition of Lymphotoxin Alpha (TNF super family, member 1) (LTA, Accession NM_{—}000595), a gene which is a cytokine that in its homotrimeric form binds to tnfrsf1a/tnfr1, tnfrsf1b/tnfbr and tnfrsf14/hvem. Accordingly, utilities of VGAM235 include diagnosis, prevention and treatment of diseases and clinical conditions associated with LTA. The function of LTA has been established by previous studies. Lymphotoxin was first characterized as a biologic factor in mitogen-stimulated lymphocytes having anticellular activity on neoplastic cell lines. It is a glycoprotein with a relative molecular mass (Mr) of 60,000-70,000, whereas monomeric lymphotoxin has an Mr of 25,000. Gray et al. (1984) isolated a chemically synthesized gene and natural complementary DNA coding for human lymphotoxin and engineered them for expression in E. coli. Cytotoxic and necrosis effects were observed in murine and human tumor cell lines in vitro and in murine sarcomas in vivo. TNF-beta (also known as lymphotoxin-alpha, or LTA) shows 35% identity and 50% homology in amino acid sequence with the TNF-alpha (OMIM Ref. No. 191160). Aggarwal et al. (1985) showed that the 2 TNFs share a common receptor on tumor cells See 191160 for information on the situation of both TNFA and TNFB on 6p. By analysis of deletions induced in lymphoblastoid cells by gamma-irradiation, Evans et al. (1989) concluded that TNFB maps to the interval between C4 and HLA-B. Spies et al. (1989) showed that the TNF-alpha and TNF-beta gene cluster is about 210 kb from HLA-B on 6p21.3. Jongeneel et al. (1991) described polymorphic microsatellites within a 12-kb region of the major histocompatibility complex that includes the TNFB locus. Lymphotoxin-alpha in a homotrimeric form is a soluble protein secreted by activated lymphocytes and presumed to act as a modulator in the immune response. The LT-alpha homotrimer shares its receptor with tumor necrosis factor and binds to both TNF receptor-1 (OMIM Ref. No. 191190) and -2 (191191

Full details of the abovementioned studies are described in the following publications, the disclosure of which are hereby incorporated by reference:
Aggarwal, B. B.; Eessalu, T. E.; Hass, P. E.: Characterization of receptors for human tumour necrosis factor and their regulation by gamma-interferon. Nature 318: 665-667, 1985.; and
Evans, A. M.; Petersen, J. W.; Sekhon, G. S.; DeMars, R.: Mapping of prolactin and tumor necrosis factor-beta genes on human chromosome 6p using lymphoblastoid cell deletion mutants. Somat.

Further studies establishing the function and utilities of LTA are found in John Hopkins OMIM database record ID 153440, and in sited publications numbered 137-14 and 834 listed in the bibliography section hereinbelow, which are also hereby incorporated by reference. Protein Phosphatase 1, Regulatory (inhibitor) Subunit 12B (PPP1R12B, Accession NM_{—}032105) is another VGAM235 host target gene. PPP1R12B BINDING SITE is HOST TARGET binding site found in the 3′ untranslated region of mRNA encoded by PPP1R12B, corresponding to a HOST TARGET binding site such as BINDING SITE I, BINDING SITE II or BINDING SITE III. Table 2 illustrates the complementarity of the nucleotide sequences of PPP1R12B BINDING SITE, designated SEQ ID:2229, to the nucleotide sequence of VGAM235 RNA, herein designated VGAM RNA, also designated SEQ ID:570.

Another function of VGAM235 is therefore inhibition of Protein Phosphatase 1, Regulatory (inhibitor) Subunit 12B (PPP1R12B, Accession NM_{—}032105). Accordingly, utilities of VGAM235 include diagnosis, prevention and treatment of diseases and clinical conditions associated with PPP1R12B. Trefoil Factor 3 (intestinal) (TFF3, Accession XM_{—}032969) is another VGAM235 host target gene. TFF3 BINDING SITE is HOST TARGET binding site found in the 3′ untranslated region of mRNA encoded by TFF3, corresponding to a HOST TARGET binding site such as BINDING SITE I, BINDING SITE II or BINDING SITE III. Table 2 illustrates the complementarity of the nucleotide sequences of TFF3 BINDING SITE, designated SEQ ID:2661, to the nucleotide sequence of VGAM235 RNA, herein designated VGAM RNA, also designated SEQ ID:570.

Another function of VGAM235 is therefore inhibition of Trefoil Factor 3 (intestinal) (TFF3, Accession XM_{—}032969), a gene which may have a role in promoting cell migration (motogen). Accordingly, utilities of VGAM235 include diagnosis, prevention and treatment of diseases and clinical conditions associated with TFF3. The function of TFF3 has been established by previous studies. Chinery et al. (1996) showed that the TFF3 gene segregates with 21q in a rodent/human somatic cell hybrid panel. By fluorescence in situ hybridization (FISH), they mapped the gene more precisely to 21q22.3. Triple FISH, together with physical mapping of human genomic DNA using pulsed field gel electrophoresis, revealed that the TFF3 gene is tightly linked to those encoding the other known human trefoil peptides, namely the breast cancer estrogen-inducible gene (TFF1; 113710) and spasmolytic protein. Using PCR analysis of a somatic cell hybrid panel and FISH using 2 large genomic recombinants cloned in a bacterial artificial chromosome (BAC), Schmitt et al. (1996) confirmed the mapping of TFF3 to 21q22.3. Seib et al. (1997) found that the TFF1, TFF2, and TFF3 genes are clustered within a 55-kb region. Burmeister and Meyer (1997) demonstrated that the mouse Tff3 gene maps to chromosome 17. Animal model experiments lend further support to the function of TFF3. Mashimo et al. (1996) produced mice unable to express the intestinal trefoil factor by means of targeted disruption of the mouse Itf gene through homologous recombination. Itf −/− mice lacked Itf protein in the colon and small intestine, and expression of the other trefoil proteins pS2 (Tff1) and SP (Tff2) was normal. Itf −/− mice developed normally. Mashimo et al. (1996) reported that although the Itf −/− mice exhibited normal mucosal architecture, their proliferative intestinal compartments were expanded and there was impaired migration of epithelium to the mucosal surface. The mice were markedly sensitive to the effects of ingested Dextran sulfate sodium (DSS) and this treatment resulted in the presence of multiple sites of ulceration and hemorrhage in colon. Histologic examination revealed large stretches of denuded epithelium and there was no evidence of reepithelization. Colons of most DSS-treated wildtype mice were unaffected. Wildtype mice did display microscopic evidence of mucosal erosion but most of the erosions were small and exhibited features of mucosal healing. Mashimo et al. (1996) demonstrated that repletion of Itf-deficient mice by luminal instillation of recombinant Itf peptide resulted in healing. Mashimo et al. (1996) concluded that their findings revealed a central role for Itf in the maintenance and repair of intestinal mucosa. They noted that trefoil factors are unusual in their resistance to acid and proteolytic enzymes, and that they therefore have potential as orally administered therapy for various forms of gastrointestinal injury, including inflammatory bowel disease.

It is appreciated that the abovementioned animal model for TFF3 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Chinery, R.; Williamson, J.; Poulsom, R.: The gene encoding human intestinal trefoil factor (TFF3) is located on chromosome 21q22.3 clustered with other members of the trefoil peptide family. Genomics 32: 281-284, 1996.; and
Mashimo, H.; Wu, D.-C.; Podolsky, D. K.; Fishman, M. C.: Impaired defense of intestinal mucosa in mice lacking intestinal trefoil factor. Science 274: 262-265, 1996.

Further studies establishing the function and utilities of TFF3 are found in John Hopkins OMIM database record ID 600633, and in sited publications numbered 1584-1585, 330, 1586-1589, 331, 1590-159 and 1626 listed in the bibliography section hereinbelow, which are also hereby incorporated by reference. Zinc-fingers and Homeoboxes 1 (ZHX1, Accession NM_{—}007222) is another VGAM235 host target gene. ZHX1 BINDING SITE is HOST TARGET binding site found in the 3′ untranslated region of mRNA encoded by ZHX1, corresponding to a HOST TARGET binding site such as BINDING SITE I, BINDING SITE II or BINDING SITE III. Table 2 illustrates the complementarity of the nucleotide sequences of ZHX1 BINDING SITE, designated SEQ ID:1366, to the nucleotide sequence of VGAM235 RNA, herein designated VGAM RNA, also designated SEQ ID:570.

Another function of VGAM235 is therefore inhibition of Zinc-fingers and Homeoboxes 1 (ZHX1, Accession NM_{—}007222). Accordingly, utilities of VGAM235 include diagnosis, prevention and treatment of diseases and clinical conditions associated with ZHX1. DKFZP434C1715 (Accession XM_{—}098421) is another VGAM235 host target gene. DKFZP434C1715 BINDING SITE is HOST TARGET binding site found in the 3′ untranslated region of mRNA encoded by DKFZP434C1715, corresponding to a HOST TARGET binding site such as BINDING SITE I, BINDING SITE II or BINDING SITE III. Table 2 illustrates the complementarity of the nucleotide sequences of DKFZP434C1715 BINDING SITE, designated SEQ ID:3371, to the nucleotide sequence of VGAM235 RNA, herein designated VGAM RNA, also designated SEQ ID:570.

Another function of VGAM235 is therefore inhibition of DKFZP434C1715 (Accession XM_{—}098421). Accordingly, utilities of VGAM235 include diagnosis, prevention and treatment of diseases and clinical conditions associated with DKFZP434C1715. FLJ21075 (Accession NM_{—}025031) is another VGAM235 host target gene. FLJ21075 BINDING SITE is HOST TARGET binding site found in the 3′ untranslated region of mRNA encoded by FLJ21075, corresponding to a HOST TARGET binding site such as BINDING SITE I, BINDING SITE II or BINDING SITE III. Table 2 illustrates the complementarity of the nucleotide sequences of FLJ21075 BINDING SITE, designated SEQ ID:2130, to the nucleotide sequence of VGAM235 RNA, herein designated VGAM RNA, also designated SEQ ID:570.

Another function of VGAM235 is therefore inhibition of FLJ21075 (Accession NM_{—}025031). Accordingly, utilities of VGAM235 include diagnosis, prevention and treatment of diseases and clinical conditions associated with FLJ21075. FLJ23556 (Accession NM_{—}024880) is another VGAM235 host target gene. FLJ23556 BINDING SITE is HOST TARGET binding site found in the 3′ untranslated region of mRNA encoded by FLJ23556, corresponding to a HOST TARGET binding site such as BINDING SITE I, BINDING SITE II or BINDING SITE III. Table 2 illustrates the complementarity of the nucleotide sequences of FLJ23556 BINDING SITE, designated SEQ ID:2101, to the nucleotide sequence of VGAM235 RNA, herein designated VGAM RNA, also designated SEQ ID:570.

Another function of VGAM235 is therefore inhibition of FLJ23556 (Accession NM_{—}024880). Accordingly, utilities of VGAM235 include diagnosis, prevention and treatment of diseases and clinical conditions associated with FLJ23556. Hydroxysteroid (17-beta) Dehydrogenase 7 (HSD17B7, Accession NM_{—}016371) is another VGAM235 host target gene. HSD17B7 BINDING SITE is HOST TARGET binding site found in the 3′ untranslated region of mRNA encoded by HSD17B7, corresponding to a HOST TARGET binding site such as BINDING SITE I, BINDING SITE II or BINDING SITE III. Table 2 illustrates the complementarity of the nucleotide sequences of HSD17B7 BINDING SITE, designated SEQ ID: 1682, to the nucleotide sequence of VGAM235 RNA, herein designated VGAM RNA, also designated SEQ ID:570.

Another function of VGAM235 is therefore inhibition of Hydroxysteroid (17-beta) Dehydrogenase 7 (HSD17B7, Accession NM_{—}016371). Accordingly, utilities of VGAM235 include diagnosis, prevention and treatment of diseases and clinical conditions associated with HSD17B7. IDN3 (Accession NM_{—}133433) is another VGAM235 host target gene. IDN3 BINDING SITE is HOST TARGET binding site found in the 3′ untranslated region of mRNA encoded by IDN3, corresponding to a HOST TARGET binding site such as BINDING SITE I, BINDING SITE II or BINDING SITE III. Table 2 illustrates the complementarity of the nucleotide sequences of IDN3 BINDING SITE, designated SEQ ID:2415, to the nucleotide sequence of VGAM235 RNA, herein designated VGAM RNA, also designated SEQ ID:570.

Another function of VGAM235 is therefore inhibition of IDN3 (Accession NM_{—}133433). Accordingly, utilities of VGAM235 include diagnosis, prevention and treatment of diseases and clinical conditions associated with IDN3. KIAA1456 (Accession XM_{—}040100) is another VGAM235 host target gene. KIAA1456 BINDING SITE is HOST TARGET binding site found in the 3′ untranslated region of mRNA encoded by KIAA1456, corresponding to a HOST TARGET binding site such as BINDING SITE I, BINDING SITE II or BINDING SITE III. Table 2 illustrates the complementarity of the nucleotide sequences of KIAA1456 BINDING SITE, designated SEQ ID:2772, to the nucleotide sequence of VGAM235 RNA, herein designated VGAM RNA, also designated SEQ ID:570.

Another function of VGAM235 is therefore inhibition of KIAA1456 (Accession XM_{—}040100). Accordingly, utilities of VGAM235 include diagnosis, prevention and treatment of diseases and clinical conditions associated with KIAA1456. Neurexophilin 3 (NXPH3, Accession XM_{—}037847) is another VGAM235 host target gene. NXPH3 BINDING SITE is HOST TARGET binding site found in the 3′ untranslated region of mRNA encoded by NXPH3, corresponding to a HOST TARGET binding site such as BINDING SITE I, BINDING SITE II or BINDING SITE III. Table 2 illustrates the complementarity of the nucleotide sequences of NXPH3 BINDING SITE, designated SEQ ID:2727, to the nucleotide sequence of VGAM235 RNA, herein designated VGAM RNA, also designated SEQ ID:570.

Another function of VGAM235 is therefore inhibition of Neurexophilin 3 (NXPH3, Accession XM_{—}037847). Accordingly, utilities of VGAM235 include diagnosis, prevention and treatment of diseases and clinical conditions associated with NXPH3. PNPASE (Accession XM_{—}048088) is another VGAM235 host target gene. PNPASE BINDING SITE is HOST TARGET binding site found in the 3′ untranslated region of mRNA encoded by PNPASE, corresponding to a HOST TARGET binding site such as BINDING SITE I, BINDING SITE II or BINDING SITE III. Table 2 illustrates the complementarity of the nucleotide sequences of PNPASE BINDING SITE, designated SEQ ID:2901, to the nucleotide sequence of VGAM235 RNA, herein designated VGAM RNA, also designated SEQ ID:570.

Another function of VGAM235 is therefore inhibition of PNPASE (Accession XM_{—}048088). Accordingly, utilities of VGAM235 include diagnosis, prevention and treatment of diseases and clinical conditions associated with PNPASE. Sprouty Homolog 4 (Drosophila) (SPRY4, Accession NM_{—}030964) is another VGAM235 host target gene. SPRY4 BINDING SITE is HOST TARGET binding site found in the 3′ untranslated region of mRNA encoded by SPRY4, corresponding to a HOST TARGET binding site such as BINDING SITE I, BINDING SITE II or BINDING SITE III. Table 2 illustrates the complementarity of the nucleotide sequences of SPRY4 BINDING SITE, designated SEQ ID:2176, to the nucleotide sequence of VGAM235 RNA, herein designated VGAM RNA, also designated SEQ ID:570.

Another function of VGAM235 is therefore inhibition of Sprouty Homolog 4 (Drosophila) (SPRY4, Accession NM_{—}030964). Accordingly, utilities of VGAM235 include diagnosis, prevention and treatment of diseases and clinical conditions associated with SPRY4. Zinc Finger Protein 33a (KOX 31) (ZNF33A, Accession XM_{—}166119) is another VGAM235 host target gene. ZNF33A BINDING SITE is HOST TARGET binding site found in the 3′ untranslated region of mRNA encoded by ZNF33A, corresponding to a HOST TARGET binding site such as BINDING SITE I, BINDING SITE II or BINDING SITE III. Table 2 illustrates the complementarity of the nucleotide sequences of ZNF33A BINDING SITE, designated SEQ ID:3519, to the nucleotide sequence of VGAM235 RNA, herein designated VGAM RNA, also designated SEQ ID:570.

Another function of VGAM235 is therefore inhibition of Zinc Finger Protein 33a (KOX 31) (ZNF33A, Accession XM_{—}166119). Accordingly, utilities of VGAM235 include diagnosis, prevention and treatment of diseases and clinical conditions associated with ZNF33A. LOC126282 (Accession XM_{—}059012) is another VGAM235 host target gene. LOC126282 BINDING SITE is HOST TARGET binding site found in the 3′ untranslated region of mRNA encoded by LOC126282, corresponding to a HOST TARGET binding site such as BINDING SITE I, BINDING SITE II or BINDING SITE III. Table 2 illustrates the complementarity of the nucleotide sequences of LOC126282 BINDING SITE, designated SEQ ID:2999, to the nucleotide sequence of VGAM235 RNA, herein designated VGAM RNA, also designated SEQ ID:570.

Another function of VGAM235 is therefore inhibition of LOC126282 (Accession XM_{—}059012). Accordingly, utilities of VGAM235 include diagnosis, prevention and treatment of diseases and clinical conditions associated with LOC126282. LOC144583 (Accession XM_{—}084907) is another VGAM235 host target gene. LOC144583 BINDING SITE is HOST TARGET binding site found in the 3′ untranslated region of mRNA encoded by LOC144583, corresponding to a HOST TARGET binding site such as BINDING SITE I, BINDING SITE II or BINDING SITE III. Table 2 illustrates the complementarity of the nucleotide sequences of LOC144583 BINDING SITE, designated SEQ ID:3068, to the nucleotide sequence of VGAM235 RNA, herein designated VGAM RNA, also designated SEQ ID:570.

Another function of VGAM235 is therefore inhibition of LOC144583 (Accession XM_{—}084907). Accordingly, utilities of VGAM235 include diagnosis, prevention and treatment of diseases and clinical conditions associated with LOC144583. LOC145231 (Accession XM_{—}096740) is another VGAM235 host target gene. LOC145231 BINDING SITE is HOST TARGET binding site found in the 3′ untranslated region of mRNA encoded by LOC145231, corresponding to a HOST TARGET binding site such as BINDING SITE I, BINDING SITE II or BINDING SITE III. Table 2 illustrates the complementarity of the nucleotide sequences of LOC145231 BINDING SITE, designated SEQ ID:3273, to the nucleotide sequence of VGAM235 RNA, herein designated VGAM RNA, also designated SEQ ID:570.

Another function of VGAM235 is therefore inhibition of LOC145231 (Accession XM_{—}096740). Accordingly, utilities of VGAM235 include diagnosis, prevention and treatment of diseases and clinical conditions associated with LOC145231. LOC147837 (Accession XM_{—}085915) is another VGAM235 host target gene. LOC147837 BINDING SITE is HOST TARGET binding site found in the 3′ untranslated region of mRNA encoded by LOC147837, corresponding to a HOST TARGET binding site such as BINDING SITE I, BINDING SITE II or BINDING SITE III. Table 2 illustrates the complementarity of the nucleotide sequences of LOC147837 BINDING SITE, designated SEQ ID:3113, to the nucleotide sequence of VGAM235 RNA, herein designated VGAM RNA, also designated SEQ ID:570.

Another function of VGAM235 is therefore inhibition of LOC147837 (Accession XM_{—}085915). Accordingly, utilities of VGAM235 include diagnosis, prevention and treatment of diseases and clinical conditions associated with LOC147837. LOC149910 (Accession XM_{—}086699) is another VGAM235 host target gene. LOC149910 BINDING SITE is HOST TARGET binding site found in the 3′ untranslated region of mRNA encoded by LOC149910, corresponding to a HOST TARGET binding site such as BINDING SITE I, BINDING SITE II or BINDING SITE III. Table 2 illustrates the complementarity of the nucleotide sequences of LOC149910 BINDING SITE, designated SEQ ID:3141, to the nucleotide sequence of VGAM235 RNA, herein designated VGAM RNA, also designated SEQ ID:570.

Another function of VGAM235 is therefore inhibition of LOC149910 (Accession XM_{—}086699). Accordingly, utilities of VGAM235 include diagnosis, prevention and treatment of diseases and clinical conditions associated with LOC149910. LOC158160 (Accession XM_{—}054490) is another VGAM235 host target gene. LOC158160 BINDING SITE is HOST TARGET binding site found in the 3′ untranslated region of mRNA encoded by LOC158160, corresponding to a HOST TARGET binding site such as BINDING SITE I, BINDING SITE II or BINDING SITE III. Table 2 illustrates the complementarity of the nucleotide sequences of LOC158160 BINDING SITE, designated SEQ ID:2967, to the nucleotide sequence of VGAM235 RNA, herein designated VGAM RNA, also designated SEQ ID:570.

Another function of VGAM235 is therefore inhibition of LOC158160 (Accession XM_{—}054490). Accordingly, utilities of VGAM235 include diagnosis, prevention and treatment of diseases and clinical conditions associated with LOC158160. LOC158292 (Accession XM_{—}098914) is another VGAM235 host target gene. LOC158292 BINDING SITE is HOST TARGET binding site found in the 5′ untranslated region of mRNA encoded by LOC158292, corresponding to a HOST TARGET binding site such as BINDING SITE I, BINDING SITE II or BINDING SITE III. Table 2 illustrates the complementarity of the nucleotide sequences of LOC158292 BINDING SITE, designated SEQ ID:3385, to the nucleotide sequence of VGAM235 RNA, herein designated VGAM RNA, also designated SEQ ID:570.

Another function of VGAM235 is therefore inhibition of LOC158292 (Accession XM_{—}098914). Accordingly, utilities of VGAM235 include diagnosis, prevention and treatment of diseases and clinical conditions associated with LOC158292. LOC162333 (Accession XM_{—}102591) is another VGAM235 host target gene. LOC162333 BINDING SITE is HOST TARGET binding site found in the 5′ untranslated region of mRNA encoded by LOC162333, corresponding to a HOST TARGET binding site such as BINDING SITE I, BINDING SITE II or BINDING SITE III. Table 2 illustrates the complementarity of the nucleotide sequences of LOC162333 BINDING SITE, designated SEQ ID:3401, to the nucleotide sequence of VGAM235 RNA, herein designated VGAM RNA, also designated SEQ ID:570.

Another function of VGAM235 is therefore inhibition of LOC162333 (Accession XM_{—}102591). Accordingly, utilities of VGAM235 include diagnosis, prevention and treatment of diseases and clinical conditions associated with LOC162333. LOC200057 (Accession XM_{—}114106) is another VGAM235 host target gene. LOC200057 BINDING SITE is HOST TARGET binding site found in the 5′ untranslated region of mRNA encoded by LOC200057, corresponding to a HOST TARGET binding site such as BINDING SITE I, BINDING SITE II or BINDING SITE III. Table 2 illustrates the complementarity of the nucleotide sequences of LOC200057 BINDING SITE, designated SEQ ID:3437, to the nucleotide sequence of VGAM235 RNA, herein designated VGAM RNA, also designated SEQ ID:570.

Another function of VGAM235 is therefore inhibition of LOC200057 (Accession XM_{—}114106). Accordingly, utilities of VGAM235 include diagnosis, prevention and treatment of diseases and clinical conditions associated with LOC200057. LOC202018 (Accession XM_{—}114420) is another VGAM235 host target gene. LOC202018 BINDING SITE is HOST TARGET binding site found in the 3′ untranslated region of mRNA encoded by LOC202018, corresponding to a HOST TARGET binding site such as BINDING SITE I, BINDING SITE II or BINDING SITE III. Table 2 illustrates the complementarity of the nucleotide sequences of LOC202018 BINDING SITE, designated SEQ ID:3460, to the nucleotide sequence of VGAM235 RNA, herein designated VGAM RNA, also designated SEQ ID:570.

Another function of VGAM235 is therefore inhibition of LOC202018 (Accession XM_{—}114420). Accordingly, utilities of VGAM235 include diagnosis, prevention and treatment of diseases and clinical conditions associated with LOC202018. LOC219392 (Accession XM_{—}165921) is another VGAM235 host target gene. LOC219392 BINDING SITE is HOST TARGET binding site found in the 5′ untranslated region of mRNA encoded by LOC219392, corresponding to a HOST TARGET binding site such as BINDING SITE I, BINDING SITE II or BINDING SITE III. Table 2 illustrates the complementarity of the nucleotide sequences of LOC219392 BINDING SITE, designated SEQ ID:3511, to the nucleotide sequence of VGAM235 RNA, herein designated VGAM RNA, also designated SEQ ID:570.

Another function of VGAM235 is therefore inhibition of LOC219392 (Accession XM_{—}165921). Accordingly, utilities of VGAM235 include diagnosis, prevention and treatment of diseases and clinical conditions associated with LOC219392. FIG. 1 further provides a conceptual description of a novel bioinformatically detected viral gene of the present invention, referred to here as Viral Genomic Address Messenger 236 (VGAM236) viral gene, which modulates expression of respective host target genes thereof, the function and utility of which host target genes is known in the art.

VGAM236 is a novel bioinformatically detected regulatory, non protein coding, viral micro RNA (miRNA) gene. The method by which VGAM236 was detected is described hereinabove with reference to FIGS. 1-8.

VGAM236 gene, herein designated VGAM GENE, is a viral gene contained in the genome of Vaccinia Virus. VGAM236 host target gene, herein designated VGAM HOST TARGET GENE, is a human gene contained in the human genome.

VGAM236 gene encodes a VGAM236 precursor RNA, herein designated VGAM PRECURSOR RNA. Similar to other miRNA genes, and unlike most ordinary genes, VGAM236 precursor RNA does not encode a protein. A nucleotide sequence identical or highly similar to the nucleotide sequence of VGAM236 precursor RNA is designated SEQ ID:222, and is provided hereinbelow with reference to the sequence listing part. Nucleotide sequence SEQ ID:222 is located at position 124655 relative to the genome of Vaccinia Virus.

VGAM236 precursor RNA folds onto itself, forming VGAM236 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM236 folded precursor RNA into VGAM236 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM236 RNA is designated SEQ ID:571, and is provided hereinbelow with reference to the sequence listing part.

VGAM236 host target gene, herein designated VGAM HOST TARGET GENE, encodes a corresponding messenger RNA, VGAM236 host target RNA, herein designated VGAM HOST TARGET RNA. VGAM236 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM236 RNA, herein designated VGAM RNA, binds complementarily to one or more host target binding sites located in untranslated regions of VGAM236 host target RNA, herein designated VGAM HOST TARGET RNA. This complementary binding is due to the fact that the nucleotide sequence of VGAM236 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM236 RNA, herein designated VGAM RNA, may have a different number of host target binding sites in untranslated regions of a VGAM236 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM236 RNA, herein designated VGAM RNA, to host target binding sites on VGAM236 host target RNA, herein designated VGAM HOST TARGET RNA, such as BINDING SITE I, BINDING SITE II and BINDING SITE III, inhibits translation of VGAM236 host target RNA into VGAM236 host target protein, herein designated VGAM HOST TARGET PROTEIN. VGAM host target protein is therefore outlined by a broken line.

It is appreciated that VGAM236 host target gene, herein designated VGAM HOST TARGET GENE, in fact represents a plurality of VGAM236 host target genes. The mRNA of each one of this plurality of VGAM236 host target genes comprises one or more host target binding sites, each having a nucleotide sequence which is at least partly complementary to VGAM236 RNA, herein designated VGAM RNA, and which when bound by VGAM236 RNA causes inhibition of translation of respective one or more VGAM236 host target proteins.

It is further appreciated by one skilled in the art that the mode of translational inhibition illustrated by FIG. 1 with specific reference to translational inhibition exerted by VGAM236 gene, herein designated VGAM GENE, on one or more VGAM236 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM236 is inhibition of expression of host target genes, as part of a novel viral mechanism of attacking a host. Accordingly, utilities of VGAM236 include diagnosis, prevention and treatment of viral infection by Vaccinia Virus. Specific functions, and accordingly utilities, of VGAM236 correlate with, and may be deduced from, the identity of the host target genes which VGAM236 binds and inhibits, and the function of these host target genes, as elaborated hereinbelow.

Nucleotide sequences of the VGAM236 precursor RNA, herein designated VGAM PRECURSOR RNA, and of the ‘diced’ VGAM236 RNA, herein designated VGAM RNA, and a schematic representation of the secondary folding of VGAM236 folded precursor RNA, herein designated VGAM FOLDED PRECURSOR RNA, of VGAM236 are further described hereinbelow with reference to Table 1.

Nucleotide sequences of host target binding sites, such as BINDING SITE-I, BINDING SITE-II and BINDING SITE-III of FIG. 1, found on VGAM236 host target RNA, and schematic representation of the complementarity of each of these host target binding sites to VGAM236 RNA, herein designated VGAM RNA, are described hereinbelow with reference to Table 2.

As mentioned hereinabove with reference to FIG. 1, a function of VGAM236 gene, herein designated VGAM is inhibition of expression of VGAM236 target genes. It is appreciated that specific functions, and accordingly utilities, of VGAM236 correlate with, and may be deduced from, the identity of the target genes which VGAM236 binds and inhibits, and the function of these target genes, as elaborated hereinbelow.

Transforming, Acidic Coiled-coil Containing Protein 1 (TACC1, Accession NM_{—}006283) is a VGAM236 host target gene. TACC1 BINDING SITE is HOST TARGET binding site found in the 3′ untranslated region of mRNA encoded by TACC1, corresponding to a HOST TARGET binding site such as BINDING SITE I, BINDING SITE II or BINDING SITE III. Table 2 illustrates the complementarity of the nucleotide sequences of TACC1 BINDING SITE, designated SEQ ID:1285, to the nucleotide sequence of VGAM236 RNA, herein designated VGAM RNA, also designated SEQ ID:571.

A function of VGAM236 is therefore inhibition of Transforming, Acidic Coiled-coil Containing Protein 1 (TACC1, Accession NM_{—}006283). Accordingly, utilities of VGAM236 include diagnosis, prevention and treatment of diseases and clinical conditions associated with TACC1. FIG. 1 further provides a conceptual description of a novel bioinformatically detected viral gene of the present invention, referred to here as Viral Genomic Address Messenger 237 (VGAM237) viral gene, which modulates expression of respective host target genes thereof, the function and utility of which host target genes is known in the art.

VGAM237 is a novel bioinformatically detected regulatory, non protein coding, viral micro RNA (miRNA) gene. The method by which VGAM237 was detected is described hereinabove with reference to FIGS. 1-8.

VGAM237 gene, herein designated VGAM GENE, is a viral gene contained in the genome of Vaccinia Virus. VGAM237 host target gene, herein designated VGAM HOST TARGET GENE, is a human gene contained in the human genome.

VGAM237 gene encodes a VGAM237 precursor RNA, herein designated VGAM PRECURSOR RNA. Similar to other miRNA genes, and unlike most ordinary genes, VGAM237 precursor RNA does not encode a protein. A nucleotide sequence identical or highly similar to the nucleotide sequence of VGAM237 precursor RNA is designated SEQ ID:223, and is provided hereinbelow with reference to the sequence listing part. Nucleotide sequence SEQ ID:223 is located at position 127745 relative to the genome of Vaccinia Virus.

VGAM237 precursor RNA folds onto itself, forming VGAM237 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM237 folded precursor RNA into VGAM237 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM237 RNA is designated SEQ ID:572, and is provided hereinbelow with reference to the sequence listing part.

VGAM237 host target gene, herein designated VGAM HOST TARGET GENE, encodes a corresponding messenger RNA, VGAM237 host target RNA, herein designated VGAM HOST TARGET RNA. VGAM237 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM237 RNA, herein designated VGAM RNA, binds complementarily to one or more host target binding sites located in untranslated regions of VGAM237 host target RNA, herein designated VGAM HOST TARGET RNA. This complementary binding is due to the fact that the nucleotide sequence of VGAM237 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM237 RNA, herein designated VGAM RNA, may have a different number of host target binding sites in untranslated regions of a VGAM237 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM237 RNA, herein designated VGAM RNA, to host target binding sites on VGAM237 host target RNA, herein designated VGAM HOST TARGET RNA, such as BINDING SITE I, BINDING SITE II and BINDING SITE III, inhibits translation of VGAM237 host target RNA into VGAM237 host target protein, herein designated VGAM HOST TARGET PROTEIN. VGAM host target protein is therefore outlined by a broken line.

It is appreciated that VGAM237 host target gene, herein designated VGAM HOST TARGET GENE, in fact represents a plurality of VGAM237 host target genes. The mRNA of each one of this plurality of VGAM237 host target genes comprises one or more host target binding sites, each having a nucleotide sequence which is at least partly complementary to VGAM237 RNA, herein designated VGAM RNA, and which when bound by VGAM237 RNA causes inhibition of translation of respective one or more VGAM237 host target proteins.

It is further appreciated by one skilled in the art that the mode of translational inhibition illustrated by FIG. 1 with specific reference to translational inhibition exerted by VGAM237 gene, herein designated VGAM GENE, on one or more VGAM237 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM237 is inhibition of expression of host target genes, as part of a novel viral mechanism of attacking a host. Accordingly, utilities of VGAM237 include diagnosis, prevention and treatment of viral infection by Vaccinia Virus. Specific functions, and accordingly utilities, of VGAM237 correlate with, and may be deduced from, the identity of the host target genes which VGAM237 binds and inhibits, and the function of these host target genes, as elaborated hereinbelow.

Nucleotide sequences of the VGAM237 precursor RNA, herein designated VGAM PRECURSOR RNA, and of the ‘diced’ VGAM237 RNA, herein designated VGAM RNA, and a schematic representation of the secondary folding of VGAM237 folded precursor RNA, herein designated VGAM FOLDED PRECURSOR RNA, of VGAM237 are further described hereinbelow with reference to Table 1.

Nucleotide sequences of host target binding sites, such as BINDING SITE-I, BINDING SITE-II and BINDING SITE-III of FIG. 1, found on VGAM237 host target RNA, and schematic representation of the complementarity of each of these host target binding sites to VGAM237 RNA, herein designated VGAM RNA, are described hereinbelow with reference to Table 2.

As mentioned hereinabove with reference to FIG. 1, a function of VGAM237 gene, herein designated VGAM is inhibition of expression of VGAM237 target genes. It is appreciated that specific functions, and accordingly utilities, of VGAM237 correlate with, and may be deduced from, the identity of the target genes which VGAM237 binds and inhibits, and the function of these target genes, as elaborated hereinbelow.

Eukaryotic Translation Initiation Factor 4 Gamma, 2 (EIF4G2, Accession NM_{—}001418) is a VGAM237 host target gene. EIF4G2 BINDING SITE is HOST TARGET binding site found in the 3′ untranslated region of mRNA encoded by EIF4G2, corresponding to a HOST TARGET binding site such as BINDING SITE I, BINDING SITE II or BINDING SITE III. Table 2 illustrates the complementarity of the nucleotide sequences of EIF4G2 BINDING SITE, designated SEQ ID:827, to the nucleotide sequence of VGAM237 RNA, herein designated VGAM RNA, also designated SEQ ID:572.

A function of VGAM237 is therefore inhibition of Eukaryotic Translation Initiation Factor 4 Gamma, 2 (EIF4G2, Accession NM_{—}001418), a gene which is a repressor of translation. Accordingly, utilities of VGAM237 include diagnosis, prevention and treatment of diseases and clinical conditions associated with EIF4G2. The function of EIF4G2 has been established by previous studies. Imataka et al. (1997) used immunoprecipitation studies with HA- or FLAG-tagged proteins to show that p97 specifically binds to EIF4A and EIF3, but not to EIF4E (OMIM Ref. No. 133440) in vitro. Transient transfection experiments showed that p97 suppressed both cap-dependent and independent translation, and that overexpression of p97 reduced overall protein synthesis. Imataka et al. (1997) suggested that p97 is a general repressor of translation that acts by forming translationally inactive complexes. Levy-Strumpf et al. (1997) showed that while a fragment of DAP5 cDNA from the C-terminal region (encoding a 28-kD ‘miniprotein’) protected cells from IFNG-induced programmed cell death at low levels of expression, higher levels of expression were toxic. They proposed that the miniprotein may be a dominant-negative inhibitor of the essential DAP5 protein, and that DAP5 may play a specific role in apoptosis.

Full details of the abovementioned studies are described in the following publications, the disclosure of which are hereby incorporated by reference:
Imataka, H.; Olsen, H. S.; Sonenberg. N.: A new translational regulator with homology to eukaryotic translation initiation factor 4G. EMBO J. 16: 817-825, 1997.; and
Levy-Strumpf, N.; Deiss, L. P.; Berissi, H.; Kimchi, A.: DAP-5, a novel homolog of eukaryotic translation initiation factor 4G isolated as a putative modulator of gamma interferon-indu.

Further studies establishing the function and utilities of EIF4G2 are found in John Hopkins OMIM database record ID 602325, and in sited publications numbered 1455-1456, 79 and 1457-1458 listed in the bibliography section hereinbelow, which are also hereby incorporated by reference. Peroxisomal Biogenesis Factor 12 (PEX12, Accession NM_{—}000286) is another VGAM237 host target gene. PEX12 BINDING SITE is HOST TARGET binding site found in the 3′ untranslated region of mRNA encoded by PEX12, corresponding to a HOST TARGET binding site such as BINDING SITE I, BINDING SITE II or BINDING SITE III. Table 2 illustrates the complementarity of the nucleotide sequences of PEX12 BINDING SITE, designated SEQ ID:727, to the nucleotide sequence of VGAM237 RNA, herein designated VGAM RNA, also designated SEQ ID:572.

Another function of VGAM237 is therefore inhibition of Peroxisomal Biogenesis Factor 12 (PEX12, Accession NM_{—}000286). Accordingly, utilities of VGAM237 include diagnosis, prevention and treatment of diseases and clinical conditions associated with PEX12. LOC130162 (Accession XM_{—}059406) is another VGAM237 host target gene. LOC130162 BINDING SITE is HOST TARGET binding site found in the 3′ untranslated region of mRNA encoded by LOC130162, corresponding to a HOST TARGET binding site such as BINDING SITE I, BINDING SITE II or BINDING SITE III. Table 2 illustrates the complementarity of the nucleotide sequences of LOC130162 BINDING SITE, designated SEQ ID:3009, to the nucleotide sequence of VGAM237 RNA, herein designated VGAM RNA, also designated SEQ ID:572.

Another function of VGAM237 is therefore inhibition of LOC130162 (Accession XM_{—}059406). Accordingly, utilities of VGAM237 include diagnosis, prevention and treatment of diseases and clinical conditions associated with LOC130162. LOC152573 (Accession XM_{—}087488) is another VGAM237 host target gene. LOC152573 BINDING SITE is HOST TARGET binding site found in the 3′ untranslated region of mRNA encoded by LOC152573, corresponding to a HOST TARGET binding site such as BINDING SITE I, BINDING SITE II or BINDING SITE III. Table 2 illustrates the complementarity of the nucleotide sequences of LOC152573 BINDING SITE, designated SEQ ID:3172, to the nucleotide sequence of VGAM237 RNA, herein designated VGAM RNA, also designated SEQ ID:572.

Another function of VGAM237 is therefore inhibition of LOC152573 (Accession XM_{—}087488). Accordingly, utilities of VGAM237 include diagnosis, prevention and treatment of diseases and clinical conditions associated with LOC152573. FIG. 1 further provides a conceptual description of a novel bioinformatically detected viral gene of the present invention, referred to here as Viral Genomic Address Messenger 238 (VGAM238) viral gene, which modulates expression of respective host target genes thereof, the function and utility of which host target genes is known in the art.

VGAM238 is a novel bioinformatically detected regulatory, non protein coding, viral micro RNA (miRNA) gene. The method by which VGAM238 was detected is described hereinabove with reference to FIGS. 1-8.

VGAM238 gene, herein designated VGAM GENE, is a viral gene contained in the genome of Vaccinia Virus. VGAM238 host target gene, herein designated VGAM HOST TARGET GENE, is a human gene contained in the human genome.

VGAM238 gene encodes a VGAM238 precursor RNA, herein designated VGAM PRECURSOR RNA. Similar to other miRNA genes, and unlike most ordinary genes, VGAM238 precursor RNA does not encode a protein. A nucleotide sequence identical or highly similar to the nucleotide sequence of VGAM238 precursor RNA is designated SEQ ID:224, and is provided hereinbelow with reference to the sequence listing part. Nucleotide sequence SEQ ID:224 is located at position 126291 relative to the genome of Vaccinia Virus.

VGAM238 precursor RNA folds onto itself, forming VGAM238 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM238 folded precursor RNA into VGAM238 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM238 RNA is designated SEQ ID:573, and is provided hereinbelow with reference to the sequence listing part.

VGAM238 host target gene, herein designated VGAM HOST TARGET GENE, encodes a corresponding messenger RNA, VGAM238 host target RNA, herein designated VGAM HOST TARGET RNA. VGAM238 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM238 RNA, herein designated VGAM RNA, binds complementarily to one or more host target binding sites located in untranslated regions of VGAM238 host target RNA, herein designated VGAM HOST TARGET RNA. This complementary binding is due to the fact that the nucleotide sequence of VGAM238 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM238 RNA, herein designated VGAM RNA, may have a different number of host target binding sites in untranslated regions of a VGAM238 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM238 RNA, herein designated VGAM RNA, to host target binding sites on VGAM238 host target RNA, herein designated VGAM HOST TARGET RNA, such as BINDING SITE I, BINDING SITE II and BINDING SITE III, inhibits translation of VGAM238 host target RNA into VGAM238 host target protein, herein designated VGAM HOST TARGET PROTEIN. VGAM host target protein is therefore outlined by a broken line.

It is appreciated that VGAM238 host target gene, herein designated VGAM HOST TARGET GENE, in fact represents a plurality of VGAM238 host target genes. The mRNA of each one of this plurality of VGAM238 host target genes comprises one or more host target binding sites, each having a nucleotide sequence which is at least partly complementary to VGAM238 RNA, herein designated VGAM RNA, and which when bound by VGAM238 RNA causes inhibition of translation of respective one or more VGAM238 host target proteins.

It is further appreciated by one skilled in the art that the mode of translational inhibition illustrated by FIG. 1 with specific reference to translational inhibition exerted by VGAM238 gene, herein designated VGAM GENE, on one or more VGAM238 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM238 is inhibition of expression of host target genes, as part of a novel viral mechanism of attacking a host. Accordingly, utilities of VGAM238 include diagnosis, prevention and treatment of viral infection by Vaccinia Virus. Specific functions, and accordingly utilities, of VGAM238 correlate with, and may be deduced from, the identity of the host target genes which VGAM238 binds and inhibits, and the function of these host target genes, as elaborated hereinbelow.

Nucleotide sequences of the VGAM238 precursor RNA, herein designated VGAM PRECURSOR RNA, and of the ‘diced’ VGAM238 RNA, herein designated VGAM RNA, and a schematic representation of the secondary folding of VGAM238 folded precursor RNA, herein designated VGAM FOLDED PRECURSOR RNA, of VGAM238 are further described hereinbelow with reference to Table 1.

Nucleotide sequences of host target binding sites, such as BINDING SITE-I, BINDING SITE-II and BINDING SITE-III of FIG. 1, found on VGAM238 host target RNA, and schematic representation of the complementarity of each of these host target binding sites to VGAM238 RNA, herein designated VGAM RNA, are described hereinbelow with reference to Table 2.

As mentioned hereinabove with reference to FIG. 1, a function of VGAM238 gene, herein designated VGAM is inhibition of expression of VGAM238 target genes. It is appreciated that specific functions, and accordingly utilities, of VGAM238 correlate with, and may be deduced from, the identity of the target genes which VGAM238 binds and inhibits, and the function of these target genes, as elaborated hereinbelow.

Fatty-acid-Coenzyme A Ligase, Long-chain 2 (FACL2, Accession NM_{—}021122) is a VGAM238 host target gene. FACL2 BINDING SITE is HOST TARGET binding site found in the 3′ untranslated region of mRNA encoded by FACL2, corresponding to a HOST TARGET binding site such as BINDING SITE I, BINDING SITE II or BINDING SITE III. Table 2 illustrates the complementarity of the nucleotide sequences of FACL2 BINDING SITE, designated SEQ ID:1942, to the nucleotide sequence of VGAM238 RNA, herein designated VGAM RNA, also designated SEQ ID:573.

A function of VGAM238 is therefore inhibition of Fatty-acid-Coenzyme A Ligase, Long-chain 2 (FACL2, Accession NM_{—}021122), a gene which activates long-chain fatty acids for both synthesis of cellular lipids. Accordingly, utilities of VGAM238 include diagnosis, prevention and treatment of diseases and clinical conditions associated with FACL2. The function of FACL2 has been established by previous studies. See 152425. Minoshima et al. (1991) isolated a human cDNA for a long-chain acyl-CoA synthetase from a human liver cDNA library using the rat cDNA as a probe. Using flow-sorted human chromosomes, they demonstrated that the gene, now designated FACL2, is located on human chromosome 4. Cantu et al. (1995) mapped FACL2 to 4q34-q35 by fluorescence in situ hybridization.

Full details of the abovementioned studies are described in the following publications, the disclosure of which are hereby incorporated by reference:
Cantu, E. S.; Sprinkle, T. J.; Ghosh, B.; Singh, I.: The human palmitoyl-CoA ligase (FACL2) gene maps to the chromosome 4q34-q35 region by fluorescence in situ hybridization (FISH) and somatic cell hybrid panels. Genomics 28: 600-602, 1995.; and
Minoshima, S.; Fukuyama, R.; Yamamoto, T.; Shimizu, N.: Mapping of human long-chain acyl-CoA synthetase to chromosome 4. (Abstract) Cytogenet. Cell Genet. 58: 1888 only, 1991.

Further studies establishing the function and utilities of FACL2 are found in John Hopkins OMIM database record ID 152426, and in sited publications numbered 792 listed in the bibliography section hereinbelow, which are also hereby incorporated by reference. Phosphotriesterase Related (PTER, Accession NM_{—}030664) is another VGAM238 host target gene. PTER BINDING SITE is HOST TARGET binding site found in the 3′ untranslated region of mRNA encoded by PTER, corresponding to a HOST TARGET binding site such as BINDING SITE I, BINDING SITE II or BINDING SITE III. Table 2 illustrates the complementarity of the nucleotide sequences of PTER BINDING SITE, designated SEQ ID:2154, to the nucleotide sequence of VGAM238 RNA, herein designated VGAM RNA, also designated SEQ ID:573.

Another function of VGAM238 is therefore inhibition of Phosphotriesterase Related (PTER, Accession NM_{—}030664), a gene which is a phosphotriesterase homology protein. Accordingly, utilities of VGAM238 include diagnosis, prevention and treatment of diseases and clinical conditions associated with PTER. The function of PTER has been established by previous studies. Microbial phosphotriesterases are a group of zinc metalloenzymes that catalyze the hydrolysis of a range of phosphotriester compounds. Davies et al. (1997) isolated rat cDNAs encoding a phosphotriesterase homolog, which they named rpr1. Using a rat rpr1 cDNA as a hybridization probe, Alimova-Kost et al. (1998) isolated human genomic sequences of PTER, a homolog of phosphotriesterases. By FISH, Alimova-Kost et al. (1998) mapped the human PTER gene to 10p12

Full details of the abovementioned studies are described in the following publications, the disclosure of which are hereby incorporated by reference:
Alimova-Kost, M. V.; Imreh, S.; Buchman, V. L.; Ninkina, N. N.: Assignment of phosphotriesterase-related gene (PTER) to human chromosome band 10p12 by in situ hybridization. Cytogenet. Cell Genet. 83: 16-17, 1998.; and
Davies, J. A.; Buchman, V. L.; Krylova, O.; Ninkina, N. N.: Molecular cloning and expression pattern of rpr-1, a resiniferatoxin-binding, phosphotriesterase-related protein, expressed.

Further studies establishing the function and utilities of PTER are found in John Hopkins OMIM database record ID 604446, and in sited publications numbered 254-255 listed in the bibliography section hereinbelow, which are also hereby incorporated by reference. ATPase, H+ Transporting, Lysosomal 13 kDa, V1 Subunit G Isoform 1 (ATP6V1G1, Accession NM_{—}004888) is another VGAM238 host target gene. ATP6V1G1 BINDING SITE is HOST TARGET binding site found in the 3′ untranslated region of mRNA encoded by ATP6V1G1, corresponding to a HOST TARGET binding site such as BINDING SITE I, BINDING SITE II or BINDING SITE III. Table 2 illustrates the complementarity of the nucleotide sequences of ATP6V1G1 BINDING SITE, designated SEQ ID:1163, to the nucleotide sequence of VGAM238 RNA, herein designated VGAM RNA, also designated SEQ ID:573.

Another function of VGAM238 is therefore inhibition of ATPase, H+ Transporting, Lysosomal 13 kDa, V1 Subunit G Isoform 1 (ATP6V1G1, Accession NM_{—}004888). Accordingly, utilities of VGAM238 include diagnosis, prevention and treatment of diseases and clinical conditions associated with ATP6V1G1. GFR (Accession NM_{—}012294) is another VGAM238 host target gene. GFR BINDING SITE is HOST TARGET binding site found in the 3′ untranslated region of mRNA encoded by GFR, corresponding to a HOST TARGET binding site such as BINDING SITE I, BINDING SITE II or BINDING SITE III. Table 2 illustrates the complementarity of the nucleotide sequences of GFR BINDING SITE, designated SEQ ID: 1423, to the nucleotide sequence of VGAM238 RNA, herein designated VGAM RNA, also designated SEQ ID:573.

Another function of VGAM238 is therefore inhibition of GFR (Accession NM_{—}012294). Accordingly, utilities of VGAM238 include diagnosis, prevention and treatment of diseases and clinical conditions associated with GFR. KIAA0349 (Accession XM_{—}166449) is another VGAM238 host target gene. KIAA0349 BINDING SITE is HOST TARGET binding site found in the 3′ untranslated region of mRNA encoded by KIAA0349, corresponding to a HOST TARGET binding site such as BINDING SITE I, BINDING SITE II or BINDING SITE III. Table 2 illustrates the complementarity of the nucleotide sequences of KIAA0349 BINDING SITE, designated SEQ ID:3562, to the nucleotide sequence of VGAM238 RNA, herein designated VGAM RNA, also designated SEQ ID:573.

Another function of VGAM238 is therefore inhibition of KIAA0349 (Accession XM_{—}166449). Accordingly, utilities of VGAM238 include diagnosis, prevention and treatment of diseases and clinical conditions associated with KIAA0349. Regulator of G-protein Signalling 20 (RGS20, Accession NM_{—}003702) is another VGAM238 host target gene. RGS20 BINDING SITE is HOST TARGET binding site found in the 3′ untranslated region of mRNA encoded by RGS20, corresponding to a HOST TARGET binding site such as BINDING SITE I, BINDING SITE II or BINDING SITE III. Table 2 illustrates the complementarity of the nucleotide sequences of RGS20 BINDING SITE, designated SEQ ID: 1049, to the nucleotide sequence of VGAM238 RNA, herein designated VGAM RNA, also designated SEQ ID:573.

Another function of VGAM238 is therefore inhibition of Regulator of G-protein Signalling 20 (RGS20, Accession NM_{—}003702). Accordingly, utilities of VGAM238 include diagnosis, prevention and treatment of diseases and clinical conditions associated with RGS20. LOC253573 (Accession XM_{—}173110) is another VGAM238 host target gene. LOC253573 BINDING SITE is HOST TARGET binding site found in the 3′ untranslated region of mRNA encoded by LOC253573, corresponding to a HOST TARGET binding site such as BINDING SITE I, BINDING SITE II or BINDING SITE III. Table 2 illustrates the complementarity of the nucleotide sequences of LOC253573 BINDING SITE, designated SEQ ID:3725, to the nucleotide sequence of VGAM238 RNA, herein designated VGAM RNA, also designated SEQ ID:573.

Another function of VGAM238 is therefore inhibition of LOC253573 (Accession XM_{—}173110). Accordingly, utilities of VGAM238 include diagnosis, prevention and treatment of diseases and clinical conditions associated with LOC253573. LOC83690 (Accession NM_{—}031461) is another VGAM238 host target gene. LOC83690 BINDING SITE is HOST TARGET binding site found in the 3′ untranslated region of mRNA encoded by LOC83690, corresponding to a HOST TARGET binding site such as BINDING SITE I, BINDING SITE II or BINDING SITE III. Table 2 illustrates the complementarity of the nucleotide sequences of LOC83690 BINDING SITE, designated SEQ ID:2203, to the nucleotide sequence of VGAM238 RNA, herein designated VGAM RNA, also designated SEQ ID:573.

Another function of VGAM238 is therefore inhibition of LOC83690 (Accession NM_{—}031461). Accordingly, utilities of VGAM238 include diagnosis, prevention and treatment of diseases and clinical conditions associated with LOC83690. FIG. 1 further provides a conceptual description of a novel bioinformatically detected viral gene of the present invention, referred to here as Viral Genomic Address Messenger 239 (VGAM239) viral gene, which modulates expression of respective host target genes thereof, the function and utility of which host target genes is known in the art.

VGAM239 is a novel bioinformatically detected regulatory, non protein coding, viral micro RNA (miRNA) gene. The method by which VGAM239 was detected is described hereinabove with reference to FIGS. 1-8.

VGAM239 gene, herein designated VGAM GENE, is a viral gene contained in the genome of Vaccinia Virus. VGAM239 host target gene, herein designated VGAM HOST TARGET GENE, is a human gene contained in the human genome.

VGAM239 gene encodes a VGAM239 precursor RNA, herein designated VGAM PRECURSOR RNA. Similar to other miRNA genes, and unlike most ordinary genes, VGAM239 precursor RNA does not encode a protein. A nucleotide sequence identical or highly similar to the nucleotide sequence of VGAM239 precursor RNA is designated SEQ ID:225, and is provided hereinbelow with reference to the sequence listing part. Nucleotide sequence SEQ ID:225 is located at position 127132 relative to the genome of Vaccinia Virus.

VGAM239 precursor RNA folds onto itself, forming VGAM239 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM239 folded precursor RNA into VGAM239 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM239 RNA is designated SEQ ID:574, and is provided hereinbelow with reference to the sequence listing part.

VGAM239 host target gene, herein designated VGAM HOST TARGET GENE, encodes a corresponding messenger RNA, VGAM239 host target RNA, herein designated VGAM HOST TARGET RNA. VGAM239 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM239 RNA, herein designated VGAM RNA, binds complementarily to one or more host target binding sites located in untranslated regions of VGAM239 host target RNA, herein designated VGAM HOST TARGET RNA. This complementary binding is due to the fact that the nucleotide sequence of VGAM239 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM239 RNA, herein designated VGAM RNA, may have a different number of host target binding sites in untranslated regions of a VGAM239 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM239 RNA, herein designated VGAM RNA, to host target binding sites on VGAM239 host target RNA, herein designated VGAM HOST TARGET RNA, such as BINDING SITE I, BINDING SITE II and BINDING SITE III, inhibits translation of VGAM239 host target RNA into VGAM239 host target protein, herein designated VGAM HOST TARGET PROTEIN. VGAM host target protein is therefore outlined by a broken line.

It is appreciated that VGAM239 host target gene, herein designated VGAM HOST TARGET GENE, in fact represents a plurality of VGAM239 host target genes. The mRNA of each one of this plurality of VGAM239 host target genes comprises one or more host target binding sites, each having a nucleotide sequence which is at least partly complementary to VGAM239 RNA, herein designated VGAM RNA, and which when bound by VGAM239 RNA causes inhibition of translation of respective one or more VGAM239 host target proteins.

It is further appreciated by one skilled in the art that the mode of translational inhibition illustrated by FIG. 1 with specific reference to translational inhibition exerted by VGAM239 gene, herein designated VGAM GENE, on one or more VGAM239 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM239 is inhibition of expression of host target genes, as part of a novel viral mechanism of attacking a host. Accordingly, utilities of VGAM239 include diagnosis, prevention and treatment of viral infection by Vaccinia Virus. Specific functions, and accordingly utilities, of VGAM239 correlate with, and may be deduced from, the identity of the host target genes which VGAM239 binds and inhibits, and the function of these host target genes, as elaborated hereinbelow.

Nucleotide sequences of the VGAM239 precursor RNA, herein designated VGAM PRECURSOR RNA, and of the ‘diced’ VGAM239 RNA, herein designated VGAM RNA, and a schematic representation of the secondary folding of VGAM239 folded precursor RNA, herein designated VGAM FOLDED PRECURSOR RNA, of VGAM239 are further described hereinbelow with reference to Table 1.

Nucleotide sequences of host target binding sites, such as BINDING SITE-I, BINDING SITE-II and BINDING SITE-III of FIG. 1, found on VGAM239 host target RNA, and schematic representation of the complementarity of each of these host target binding sites to VGAM239 RNA, herein designated VGAM RNA, are described hereinbelow with reference to Table 2.

As mentioned hereinabove with reference to FIG. 1, a function of VGAM239 gene, herein designated VGAM is inhibition of expression of VGAM239 target genes. It is appreciated that specific functions, and accordingly utilities, of VGAM239 correlate with, and may be deduced from, the identity of the target genes which VGAM239 binds and inhibits, and the function of these target genes, as elaborated hereinbelow.

RNA Binding Motif Protein 8A (RBM8A, Accession NM_{—}005105) is a VGAM239 host target gene. RBM8A BINDING SITE is HOST TARGET binding site found in the 3′ untranslated region of mRNA encoded by RBM8A, corresponding to a HOST TARGET binding site such as BINDING SITE I, BINDING SITE II or BINDING SITE III. Table 2 illustrates the complementarity of the nucleotide sequences of RBM8A BINDING SITE, designated SEQ ID:1186, to the nucleotide sequence of VGAM239 RNA, herein designated VGAM RNA, also designated SEQ ID:574.

A function of VGAM239 is therefore inhibition of RNA Binding Motif Protein 8A (RBM8A, Accession NM_{—}005105), a gene which involves in the pathway of gene expression postsplicing nuclear preexport mRNPs, and newly exported cytoplasmic mRNPs. Accordingly, utilities of VGAM239 include diagnosis, prevention and treatment of diseases and clinical conditions associated with RBM8A. The function of RBM8A has been established by previous studies. Mago nashi (MAGOH; 602603), meaning grand-childless, is the homolog of a Drosophila protein required for normal germ plasm development in fly embryos. By performing a yeast 2-hybrid screen on a fetal brain cDNA library with MAGOH as the bait, Zhao et al. (2000) recovered a cDNA encoding RBM8. The 173-amino acid RBM8 protein is more than 93% identical to the mouse and zebrafish sequences, and the mouse differences are all accounted for by an 11-amino acid N-terminal insertion and another single-residue insertion in the mouse sequence. Exchange partner and GST pull-down assays confirmed the MAGOH-RBM8 interaction and showed that RBM8 is expressed as a 26-kD protein, slightly larger than the predicted mass of 23 kD. Northern blot analysis detected a major RBM8 transcript of less than 1.0 kb in all tissues tested, with weakest expression in pancreas and brain. By searching an EST database for homologs of the gonadotropin-releasing hormone receptor (GNRHR; 138850), followed by 5-prime RACE on a skeletal muscle cDNA library, Conklin et al. (2000) identified a cDNA encoding RBM8. Northern blot analysis detected a major 0.9-kb transcript in all tissues tested. Sequence analysis of the 174-amino acid protein predicted an RNA-binding domain, which is composed of 2 amphipathic alpha helices packed against a 4-stranded beta sheet, and a C-terminal arg-rich segment.

Full details of the abovementioned studies are described in the following publications, the disclosure of which are hereby incorporated by reference:
Conklin, D. C.; Rixon, M. W.; Kuestner, R. E.; Maurer, M. F.; Whitmore, T. E.; Millar, R. P.: Cloning and gene expression of a novel human ribonucleoprotein. Biochim. Biophys. Acta 1492: 465-469, 2000.; and
Zhao, X.-F.; Nowak, N. J.; Shows, T. B.; Aplan, P. D.: MAGOH interacts with a novel RNA-binding protein. Genomics 63: 145-148, 2000.

Further studies establishing the function and utilities of RBM8A are found in John Hopkins OMIM database record ID 605313, and in sited publications numbered 1674-1677 listed in the bibliography section hereinbelow, which are also hereby incorporated by reference. Solute Carrier Family 7 (cationic amino acid transporter, y+ system), Member 6 (SLC7A6, Accession NM_{—}003983) is another VGAM239 host target gene. SLC7A6 BINDING SITE is HOST TARGET binding site found in the 3′ untranslated region of mRNA encoded by SLC7A6, corresponding to a HOST TARGET binding site such as BINDING SITE I, BINDING SITE II or BINDING SITE III. Table 2 illustrates the complementarity of the nucleotide sequences of SLC7A6 BINDING SITE, designated SEQ ID: 1078, to the nucleotide sequence of VGAM239 RNA, herein designated VGAM RNA, also designated SEQ ID:574.

Another function of VGAM239 is therefore inhibition of Solute Carrier Family 7 (cationic amino acid transporter, y+ system), Member 6 (SLC7A6, Accession NM_{—}003983), a gene which is involved in mediating amino acid transport. Accordingly, utilities of VGAM239 include diagnosis, prevention and treatment of diseases and clinical conditions associated with SLC7A6. The function of SLC7A6 and its association with various diseases and clinical conditions, has been established by previous studies, as described hereinabove with reference to VGAM48. Sema Domain, Immunoglobulin Domain (Ig), Short Basic Domain, Secreted, (semaphorin) 3E (SEMA3E, Accession NM_{—}012431) is another VGAM239 host target gene. SEMA3E BINDING SITE is HOST TARGET binding site found in the 3′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1432, to the nucleotide sequence of VGAM239 RNA, herein designated VGAM RNA, also designated SEQ ID:574.

Another function of VGAM239 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM239 include diagnosis, prevention and treatment of diseases and clinical conditions associated with SEMA3E. LOC151201 (Accession XM_{—}098021) is another VGAM239 host target gene. LOC151201 BINDING SITE is HOST TARGET binding site found in the 3′ untranslated region of mRNA encoded by LOC151201, corresponding to a HOST TARGET binding site such as BINDING SITE I, BINDING SITE II or BINDING SITE III. Table 2 illustrates the complementarity of the nucleotide sequences of LOC151201 BINDING SITE, designated SEQ ID:3339, to the nucleotide sequence of VGAM239 RNA, herein designated VGAM RNA, also designated SEQ ID:574.

Another function of VGAM239 is therefore inhibition of LOC151201 (Accession XM_{—}098021). Accordingly, utilities of VGAM239 include diagnosis, prevention and treatment of diseases and clinical conditions associated with LOC151201. LOC90979 (Accession XM_{—}035323) is another VGAM239 host target gene. LOC90979 BINDING SITE is HOST TARGET binding site found in the 3′ untranslated region of mRNA encoded by LOC90979, corresponding to a HOST TARGET binding site such as BINDING SITE I, BINDING SITE II or BINDING SITE III. Table 2 illustrates the complementarity of the nucleotide sequences of LOC90979 BINDING SITE, designated SEQ ID:2692, to the nucleotide sequence of VGAM239 RNA, herein designated VGAM RNA, also designated SEQ ID:574.

Another function of VGAM239 is therefore inhibition of LOC90979 (Accession XM_{—}035323). Accordingly, utilities of VGAM239 include diagnosis, prevention and treatment of diseases and clinical conditions associated with LOC90979. FIG. 1 further provides a conceptual description of a novel bioinformatically detected viral gene of the present invention, referred to here as Viral Genomic Address Messenger 240 (VGAM240) viral gene, which modulates expression of respective host target genes thereof, the function and utility of which host target genes is known in the art.

VGAM240 is a novel bioinformatically detected regulatory, non protein coding, viral micro RNA (miRNA) gene. The method by which VGAM240 was detected is described hereinabove with reference to FIGS. 1-8.

VGAM240 gene, herein designated VGAM GENE, is a viral gene contained in the genome of Vaccinia Virus. VGAM240 host target gene, herein designated VGAM HOST TARGET GENE, is a human gene contained in the human genome.

VGAM240 gene encodes a VGAM240 precursor RNA, herein designated VGAM PRECURSOR RNA. Similar to other miRNA genes, and unlike most ordinary genes, VGAM240 precursor RNA does not encode a protein. A nucleotide sequence identical or highly similar to the nucleotide sequence of VGAM240 precursor RNA is designated SEQ ID:226, and is provided hereinbelow with reference to the sequence listing part. Nucleotide sequence SEQ ID:226 is located at position 129602 relative to the genome of Vaccinia Virus.

VGAM240 precursor RNA folds onto itself, forming VGAM240 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM240 folded precursor RNA into VGAM240 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM240 RNA is designated SEQ ID:575, and is provided hereinbelow with reference to the sequence listing part.

VGAM240 host target gene, herein designated VGAM HOST TARGET GENE, encodes a corresponding messenger RNA, VGAM240 host target RNA, herein designated VGAM HOST TARGET RNA. VGAM240 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM240 RNA, herein designated VGAM RNA, binds complementarily to one or more host target binding sites located in untranslated regions of VGAM240 host target RNA, herein designated VGAM HOST TARGET RNA. This complementary binding is due to the fact that the nucleotide sequence of VGAM240 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM240 RNA, herein designated VGAM RNA, may have a different number of host target binding sites in untranslated regions of a VGAM240 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM240 RNA, herein designated VGAM RNA, to host target binding sites on VGAM240 host target RNA, herein designated VGAM HOST TARGET RNA, such as BINDING SITE I, BINDING SITE II and BINDING SITE III, inhibits translation of VGAM240 host target RNA into VGAM240 host target protein, herein designated VGAM HOST TARGET PROTEIN. VGAM host target protein is therefore outlined by a broken line.

It is appreciated that VGAM240 host target gene, herein designated VGAM HOST TARGET GENE, in fact represents a plurality of VGAM240 host target genes. The mRNA of each one of this plurality of VGAM240 host target genes comprises one or more host target binding sites, each having a nucleotide sequence which is at least partly complementary to VGAM240 RNA, herein designated VGAM RNA, and which when bound by VGAM240 RNA causes inhibition of translation of respective one or more VGAM240 host target proteins.

It is further appreciated by one skilled in the art that the mode of translational inhibition illustrated by FIG. 1 with specific reference to translational inhibition exerted by VGAM240 gene, herein designated VGAM GENE, on one or more VGAM240 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM240 is inhibition of expression of host target genes, as part of a novel viral mechanism of attacking a host. Accordingly, utilities of VGAM240 include diagnosis, prevention and treatment of viral infection by Vaccinia Virus. Specific functions, and accordingly utilities, of VGAM240 correlate with, and may be deduced from, the identity of the host target genes which VGAM240 binds and inhibits, and the function of these host target genes, as elaborated hereinbelow.

Nucleotide sequences of the VGAM240 precursor RNA, herein designated VGAM PRECURSOR RNA, and of the ‘diced’ VGAM240 RNA, herein designated VGAM RNA, and a schematic representation of the secondary folding of VGAM240 folded precursor RNA, herein designated VGAM FOLDED PRECURSOR RNA, of VGAM240 are further described hereinbelow with reference to Table 1.

Nucleotide sequences of host target binding sites, such as BINDING SITE-I, BINDING SITE-II and BINDING SITE-III of FIG. 1, found on VGAM240 host target RNA, and schematic representation of the complementarity of each of these host target binding sites to VGAM240 RNA, herein designated VGAM RNA, are described hereinbelow with reference to Table 2.

As mentioned hereinabove with reference to FIG. 1, a function of VGAM240 gene, herein designated VGAM is inhibition of expression of VGAM240 target genes. It is appreciated that specific functions, and accordingly utilities, of VGAM240 correlate with, and may be deduced from, the identity of the target genes which VGAM240 binds and inhibits, and the function of these target genes, as elaborated hereinbelow.

Zic Family Member 1 (odd-paired homolog, Drosophila) (ZIC1, Accession NM_{—}003412) is a VGAM240 host target gene. ZIC1 BINDING SITE is HOST TARGET binding site found in the 3′ untranslated region of mRNA encoded by ZIC1, corresponding to a HOST TARGET binding site such as BINDING SITE I, BINDING SITE II or BINDING SITE III. Table 2 illustrates the complementarity of the nucleotide sequences of ZIC1 BINDING SITE, designated SEQ ID:1017, to the nucleotide sequence of VGAM240 RNA, herein designated VGAM RNA, also designated SEQ ID:575.

A function of VGAM240 is therefore inhibition of Zic Family Member 1 (odd-paired homolog, Drosophila) (ZIC1, Accession NM_{—}003412), a gene which may play a role in cerebellar development. Accordingly, utilities of VGAM240 include diagnosis, prevention and treatment of diseases and clinical conditions associated with ZIC1. The function of ZIC1 has been established by previous studies. mouse cerebellum and is highly homologous to the Drosophila pair-rule gene Opa. To clarify the mechanism for the development of the human cerebellum and the possible involvement of ZIC in human nervous system diseases, Yokota et al. (1996) isolated human ZIC cDNA and examined its expression by using monoclonal antibody against recombinant ZIC protein. The nucleotide sequence of human ZIC cDNA is 85% homologous to that the mouse zic gene. Its putative amino acid sequence is highly conserved (more than 99%) except for substitution of only 2 amino acid residues. By fluorescence in situ hybridization, Yokota et al. (1996) mapped the human ZIC gene to 3q24. The human ZIC protein was immunohistochemically detected in the nuclei of the cerebellar granule cell lineage from the progenitor cells of the external germinal layer to the postmigrated cells of the internal granular layer. Furthermore, ZIC protein was detected in medulloblastoma (26 of 29 cases), whereas none of 70 other tumors examined, including primitive neuroectodermal tumors, expressed this protein. These findings suggested that ZIC is a potential biomarker for medulloblastoma as well as the human cerebellar granule cell lineage.

Full details of the abovementioned studies are described in the following publications, the disclosure of which are hereby incorporated by reference:
Aruga, J.; Yokota, N.; Hashimoto, M.; Furuichi, T.; Fukuda, M.; Mikoshiba, K.: A novel zinc finger protein, Zic, is involved in neurogenesis, especially in the cell lineage of cerebellar granule cells. J. Neurochem. 63: 1880-1890, 1994.; and
Yokota, N.; Aruga, J.; Takai, S.; Yamada, K.; Hamazaki, M.; Iwase, T.; Sugimura, H.; Mikoshiba, K.: Predominant expression of human Zic in cerebellar granule cell lineage and medulloblasto.

Further studies establishing the function and utilities of ZIC1 are found in John Hopkins OMIM database record ID 600470, and in sited publications numbered 1764-1766 listed in the bibliography section hereinbelow, which are also hereby incorporated by reference. KIAA1550 (Accession XM_{—}039393) is another VGAM240 host target gene. KIAA1550 BINDING SITE is HOST TARGET binding site found in the 3′ untranslated region of mRNA encoded by KIAA1550, corresponding to a HOST TARGET binding site such as BINDING SITE I, BINDING SITE II or BINDING SITE III. Table 2 illustrates the complementarity of the nucleotide sequences of KIAA1550 BINDING SITE, designated SEQ ID:2752, to the nucleotide sequence of VGAM240 RNA, herein designated VGAM RNA, also designated SEQ ID:575.

Another function of VGAM240 is therefore inhibition of KIAA1550 (Accession XM_{—}039393). Accordingly, utilities of VGAM240 include diagnosis, prevention and treatment of diseases and clinical conditions associated with KIAA1550. LOC143720 (Accession XM_{—}017350) is another VGAM240 host target gene. LOC143720 BINDING SITE is HOST TARGET binding site found in the 3′ untranslated region of mRNA encoded by LOC143720, corresponding to a HOST TARGET binding site such as BINDING SITE I, BINDING SITE II or BINDING SITE III. Table 2 illustrates the complementarity of the nucleotide sequences of LOC143720 BINDING SITE, designated SEQ ID:2568, to the nucleotide sequence of VGAM240 RNA, herein designated VGAM RNA, also designated SEQ ID:575.

Another function of VGAM240 is therefore inhibition of LOC143720 (Accession XM_{—}017350). Accordingly, utilities of VGAM240 include diagnosis, prevention and treatment of diseases and clinical conditions associated with LOC143720. LOC150848 (Accession XM_{—}097959) is another VGAM240 host target gene. LOC150848 BINDING SITE is HOST TARGET binding site found in the 5′ untranslated region of mRNA encoded by LOC150848, corresponding to a HOST TARGET binding site such as BINDING SITE I, BINDING SITE II or BINDING SITE III. Table 2 illustrates the complementarity of the nucleotide sequences of LOC150848 BINDING SITE, designated SEQ ID:3335, to the nucleotide sequence of VGAM240 RNA, herein designated VGAM RNA, also designated SEQ ID:575.

Another function of VGAM240 is therefore inhibition of LOC150848 (Accession XM_{—}097959). Accordingly, utilities of VGAM240 include diagnosis, prevention and treatment of diseases and clinical conditions associated with LOC150848. FIG. 1 further provides a conceptual description of a novel bioinformatically detected viral gene of the present invention, referred to here as Viral Genomic Address Messenger 241 (VGAM241) viral gene, which modulates expression of respective host target genes thereof, the function and utility of which host target genes is known in the art.

VGAM241 is a novel bioinformatically detected regulatory, non protein coding, viral micro RNA (miRNA) gene. The method by which VGAM241 was detected is described hereinabove with reference to FIGS. 1-8.

VGAM241 gene, herein designated VGAM GENE, is a viral gene contained in the genome of Vaccinia Virus. VGAM241 host target gene, herein designated VGAM HOST TARGET GENE, is a human gene contained in the human genome.

VGAM241 gene encodes a VGAM241 precursor RNA, herein designated VGAM PRECURSOR RNA. Similar to other miRNA genes, and unlike most ordinary genes, VGAM241 precursor RNA does not encode a protein. A nucleotide sequence identical or highly similar to the nucleotide sequence of VGAM241 precursor RNA is designated SEQ ID:227, and is provided hereinbelow with reference to the sequence listing part. Nucleotide sequence SEQ ID:227 is located at position 130612 relative to the genome of Vaccinia Virus.

VGAM241 precursor RNA folds onto itself, forming VGAM241 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM241 folded precursor RNA into VGAM241 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM241 RNA is designated SEQ ID:576, and is provided hereinbelow with reference to the sequence listing part.

VGAM241 host target gene, herein designated VGAM HOST TARGET GENE, encodes a corresponding messenger RNA, VGAM241 host target RNA, herein designated VGAM HOST TARGET RNA. VGAM241 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM241 RNA, herein designated VGAM RNA, binds complementarily to one or more host target binding sites located in untranslated regions of VGAM241 host target RNA, herein designated VGAM HOST TARGET RNA. This complementary binding is due to the fact that the nucleotide sequence of VGAM241 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM241 RNA, herein designated VGAM RNA, may have a different number of host target binding sites in untranslated regions of a VGAM241 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM241 RNA, herein designated VGAM RNA, to host target binding sites on VGAM241 host target RNA, herein designated VGAM HOST TARGET RNA, such as BINDING SITE I, BINDING SITE II and BINDING SITE III, inhibits translation of VGAM241 host target RNA into VGAM241 host target protein, herein designated VGAM HOST TARGET PROTEIN. VGAM host target protein is therefore outlined by a broken line.

It is appreciated that VGAM241 host target gene, herein designated VGAM HOST TARGET GENE, in fact represents a plurality of VGAM241 host target genes. The mRNA of each one of this plurality of VGAM241 host target genes comprises one or more host target binding sites, each having a nucleotide sequence which is at least partly complementary to VGAM241 RNA, herein designated VGAM RNA, and which when bound by VGAM241 RNA causes inhibition of translation of respective one or more VGAM241 host target proteins.

It is further appreciated by one skilled in the art that the mode of translational inhibition illustrated by FIG. 1 with specific reference to translational inhibition exerted by VGAM241 gene, herein designated VGAM GENE, on one or more VGAM241 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM241 is inhibition of expression of host target genes, as part of a novel viral mechanism of attacking a host. Accordingly, utilities of VGAM241 include diagnosis, prevention and treatment of viral infection by Vaccinia Virus. Specific functions, and accordingly utilities, of VGAM241 correlate with, and may be deduced from, the identity of the host target genes which VGAM241 binds and inhibits, and the function of these host target genes, as elaborated hereinbelow.

Nucleotide sequences of the VGAM241 precursor RNA, herein designated VGAM PRECURSOR RNA, and of the ‘diced’ VGAM241 RNA, herein designated VGAM RNA, and a schematic representation of the secondary folding of VGAM241 folded precursor RNA, herein designated VGAM FOLDED PRECURSOR RNA, of VGAM241 are further described hereinbelow with reference to Table 1.

Nucleotide sequences of host target binding sites, such as BINDING SITE-I, BINDING SITE-II and BINDING SITE-III of FIG. 1, found on VGAM241 host target RNA, and schematic representation of the complementarity of each of these host target binding sites to VGAM241 RNA, herein designated VGAM RNA, are described hereinbelow with reference to Table 2.

As mentioned hereinabove with reference to FIG. 1, a function of VGAM241 gene, herein designated VGAM is inhibition of expression of VGAM241 target genes. It is appreciated that specific functions, and accordingly utilities, of VGAM241 correlate with, and may be deduced from, the identity of the target genes which VGAM241 binds and inhibits, and the function of these target genes, as elaborated hereinbelow.

MAIL (Accession NM_{—}031419) is a VGAM241 host target gene. MAIL BINDING SITE is HOST TARGET binding site found in the 3′ untranslated region of mRNA encoded by MAIL, corresponding to a HOST TARGET binding site such as BINDING SITE I, BINDING SITE II or BINDING SITE III. Table 2 illustrates the complementarity of the nucleotide sequences of MAIL BINDING SITE, designated SEQ ID:2194, to the nucleotide sequence of VGAM241 RNA, herein designated VGAM RNA, also designated SEQ ID:576.

A function of VGAM241 is therefore inhibition of MAIL (Accession NM_{—}031419). Accordingly, utilities of VGAM241 include diagnosis, prevention and treatment of diseases and clinical conditions associated with MAIL. LOC151414 (Accession XM_{—}087197) is another VGAM241 host target gene. LOC151414 BINDING SITE is HOST TARGET binding site found in the 3′ untranslated region of mRNA encoded by LOC151414, corresponding to a HOST TARGET binding site such as BINDING SITE I, BINDING SITE II or BINDING SITE III. Table 2 illustrates the complementarity of the nucleotide sequences of LOC151414 BINDING SITE, designated SEQ ID:3157, to the nucleotide sequence of VGAM241 RNA, herein designated VGAM RNA, also designated SEQ ID:576.

Another function of VGAM241 is therefore inhibition of LOC151414 (Accession XM_{—}087197). Accordingly, utilities of VGAM241 include diagnosis, prevention and treatment of diseases and clinical conditions associated with LOC151414. LOC155038 (Accession XM_{—}088130) is another VGAM241 host target gene. LOC155038 BINDING SITE is HOST TARGET binding site found in the 3′ untranslated region of mRNA encoded by LOC155038, corresponding to a HOST TARGET binding site such as BINDING SITE I, BINDING SITE II or BINDING SITE III. Table 2 illustrates the complementarity of the nucleotide sequences of LOC155038 BINDING SITE, designated SEQ ID:3197, to the nucleotide sequence of VGAM241 RNA, herein designated VGAM RNA, also designated SEQ ID:576.

Another function of VGAM241 is therefore inhibition of LOC155038 (Accession XM_{—}088130). Accordingly, utilities of VGAM241 include diagnosis, prevention and treatment of diseases and clinical conditions associated with LOC155038. FIG. 1 further provides a conceptual description of a novel bioinformatically detected viral gene of the present invention, referred to here as Viral Genomic Address Messenger 242 (VGAM242) viral gene, which modulates expression of respective host target genes thereof, the function and utility of which host target genes is known in the art.

VGAM242 is a novel bioinformatically detected regulatory, non protein coding, viral micro RNA (miRNA) gene. The method by which VGAM242 was detected is described hereinabove with reference to FIGS. 1-8.

VGAM242 gene, herein designated VGAM GENE, is a viral gene contained in the genome of Vaccinia Virus. VGAM242 host target gene, herein designated VGAM HOST TARGET GENE, is a human gene contained in the human genome.

VGAM242 gene encodes a VGAM242 precursor RNA, herein designated VGAM PRECURSOR RNA. Similar to other miRNA genes, and unlike most ordinary genes, VGAM242 precursor RNA does not encode a protein. A nucleotide sequence identical or highly similar to the nucleotide sequence of VGAM242 precursor RNA is designated SEQ ID:228, and is provided hereinbelow with reference to the sequence listing part. Nucleotide sequence SEQ ID:228 is located at position 131407 relative to the genome of Vaccinia Virus.

VGAM242 precursor RNA folds onto itself, forming VGAM242 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM242 folded precursor RNA into VGAM242 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM242 RNA is designated SEQ ID:577, and is provided hereinbelow with reference to the sequence listing part.

VGAM242 host target gene, herein designated VGAM HOST TARGET GENE, encodes a corresponding messenger RNA, VGAM242 host target RNA, herein designated VGAM HOST TARGET RNA. VGAM242 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM242 RNA, herein designated VGAM RNA, binds complementarily to one or more host target binding sites located in untranslated regions of VGAM242 host target RNA, herein designated VGAM HOST TARGET RNA. This complementary binding is due to the fact that the nucleotide sequence of VGAM242 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM242 RNA, herein designated VGAM RNA, may have a different number of host target binding sites in untranslated regions of a VGAM242 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM242 RNA, herein designated VGAM RNA, to host target binding sites on VGAM242 host target RNA, herein designated VGAM HOST TARGET RNA, such as BINDING SITE I, BINDING SITE II and BINDING SITE III, inhibits translation of VGAM242 host target RNA into VGAM242 host target protein, herein designated VGAM HOST TARGET PROTEIN. VGAM host target protein is therefore outlined by a broken line.

It is appreciated that VGAM242 host target gene, herein designated VGAM HOST TARGET GENE, in fact represents a plurality of VGAM242 host target genes. The mRNA of each one of this plurality of VGAM242 host target genes comprises one or more host target binding sites, each having a nucleotide sequence which is at least partly complementary to VGAM242 RNA, herein designated VGAM RNA, and which when bound by VGAM242 RNA causes inhibition of translation of respective one or more VGAM242 host target proteins.

It is further appreciated by one skilled in the art that the mode of translational inhibition illustrated by FIG. 1 with specific reference to translational inhibition exerted by VGAM242 gene, herein designated VGAM GENE, on one or more VGAM242 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM242 is inhibition of expression of host target genes, as part of a novel viral mechanism of attacking a host. Accordingly, utilities of VGAM242 include diagnosis, prevention and treatment of viral infection by Vaccinia Virus. Specific functions, and accordingly utilities, of VGAM242 correlate with, and may be deduced from, the identity of the host target genes which VGAM242 binds and inhibits, and the function of these host target genes, as elaborated hereinbelow.

Nucleotide sequences of the VGAM242 precursor RNA, herein designated VGAM PRECURSOR RNA, and of the ‘diced’ VGAM242 RNA, herein designated VGAM RNA, and a schematic representation of the secondary folding of VGAM242 folded precursor RNA, herein designated VGAM FOLDED PRECURSOR RNA, of VGAM242 are further described hereinbelow with reference to Table 1.

Nucleotide sequences of host target binding sites, such as BINDING SITE-I, BINDING SITE-II and BINDING SITE-III of FIG. 1, found on VGAM242 host target RNA, and schematic representation of the complementarity of each of these host target binding sites to VGAM242 RNA, herein designated VGAM RNA, are described hereinbelow with reference to Table 2.

As mentioned hereinabove with reference to FIG. 1, a function of VGAM242 gene, herein designated VGAM is inhibition of expression of VGAM242 target genes. It is appreciated that specific functions, and accordingly utilities, of VGAM242 correlate with, and may be deduced from, the identity of the target genes which VGAM242 binds and inhibits, and the function of these target genes, as elaborated hereinbelow.

Corticotropin Releasing Hormone (CRH, Accession NM_{—}000756) is a VGAM242 host target gene. CRH BINDING SITE is HOST TARGET binding site found in the 3′ untranslated region of mRNA encoded by CRH, corresponding to a HOST TARGET binding site such as BINDING SITE I, BINDING SITE II or BINDING SITE III. Table 2 illustrates the complementarity of the nucleotide sequences of CRH BINDING SITE, designated SEQ ID:772, to the nucleotide sequence of VGAM242 RNA, herein designated VGAM RNA, also designated SEQ ID:577.

A function of VGAM242 is therefore inhibition of Corticotropin Releasing Hormone (CRH, Accession NM_{—}000756), a gene which regulates the release of corticotropin from pituitary gland. Accordingly, utilities of VGAM242 include diagnosis, prevention and treatment of diseases and clinical conditions associated with CRH. The function of CRH has been established by previous studies. Response to stress in mammals requires an intact hypothalamic-pituitary-adrenal axis. The proximal part of the response is mediated by secretion of corticotropin-releasing hormone (CRH) by the paraventricular nucleus of the hypothalamus. CRH is a 41-amino acid peptide derived by enzymatic cleavage from a 191-amino acid preprohormone. Shibahara et al. (1983) cloned and sequenced the human CRH gene. Arbiser et al. (1988) assigned the gene for CRH to 8q13 by somatic cell hybrid and in situ hybridization studies. The absence of secondary hybridization strongly suggested that hypothalamic and placental CRH are transcribed from the same gene. Kellogg et al. (1989) corroborated the assignment to 8q13 by in situ hybridization. Knapp et al. (1993) showed that the homologous gene is located on mouse chromosome 3 Sebaceous glands may be involved in a pathway conceptually similar to that of the hypothalamic-pituitary-adrenal (HPA) axis. CRH is the most proximal element of the HPA axis, and it acts as a central coordinator for neuroendocrine and behavioral responses to stress. To examine the probability of an HPA equivalent pathway in sebaceous glands, Zouboulis et al. (2002) investigated the expression of CRH, CRH-binding protein, CRHBP (OMIM Ref. No. 122559), and CRH receptors (CRHR1, 122561 and CRHR2, 602034) in sebocytes in vitro and their regulation by CRH and several other hormones. CRHR1 was the predominant type, being twice as abundant as CRHR2. CRH was biologically active on human sebocytes; it induced biphasic increase in synthesis of sebaceous lipids, although it did not affect cell viability, cell proliferation, or IL1B (OMIM Ref. No. 147720)-induced IL8 (OMIM Ref. No. 146930) release. Zouboulis et al. (2002) interpreted these and other findings as indicating that CRH may be an autocrine hormone for human sebocytes that exerts homeostatic lipogenic activity, whereas testosterone and growth hormone induced CRH negative feedback. The findings implicated CRH in the clinical development of acne, seborrhea, androgenetic alopecia, skin aging, xerosis, and other skin disorders associated with alterations in lipid formation of sebaceous origin Animal model experiments lend further support to the function of CRH. In adult male rhesus macaques, Habib et al. (2000) evaluated the effects of a lipophilic nonpeptide antagonist to CRH type 1 receptor, antalarmin, on the behavioral, neuroendocrine, and autonomic components of the stress response. After oral administration, significant antalarmin concentrations were detected in the systemic circulation and the cerebrospinal fluid. The monkeys were exposed to an intense social stressor, namely, placement of 2 unfamiliar males in adjacent cages separated only by a transparent Plexiglas screen. Antalarmin significantly inhibited a repertoire of behaviors associated with anxiety and fear, such as body tremors, grimacing, teeth gnashing, urination, and defecation. In contrast, antalarmin increased exploratory and sexual behaviors that are normally suppressed during stress. Moreover, antalarmin significantly diminished the increases in cerebrospinal fluid CRH as well as the pituitary-adrenal, sympathetic, and adrenal medullary responses to stress. Habib et al. (2000) suggested that a CRH type 1 receptor antagonist may be of therapeutic value in human psychiatric, reproductive, and cardiovascular disorders associated with CRH system hyperactivity.

It is appreciated that the abovementioned animal model for CRH is acknowledged by those skilled in the art as a scientifically valid animal model, as can be further appreciated from the publications sited hereinbelow.

Full details of the abovementioned studies are described in the following publications, the disclosure of which are hereby incorporated by reference:
Knapp, L. T.; Keegan, C. E.; Seasholtz, A. F.; Camper, S. A.: Corticotropin-releasing hormone (Crh) maps to mouse chromosome 3. Mammalian Genome 4: 615-617, 1993.; and
Habib, K. E.; Weld, K. P.; Rice, K. C.; Pushkas, J.; Champoux, M.; Listwak, S.; Webster, E. L.; Atkinson, A. J.; Schulkin, J.; Contoreggi, C.; Chrousos, G. P.; McCann, S. M.; Suomi, S. J.

Further studies establishing the function and utilities of CRH are found in John Hopkins OMIM database record ID 122560, and in sited publications numbered 42 and 457-440 listed in the bibliography section hereinbelow, which are also hereby incorporated by reference. Chromosome 1 Open Reading Frame 24 (Clorf24, Accession NM_{—}052966) is another VGAM242 host target gene. Clorf24 BINDING SITE is HOST TARGET binding site found in the 3′ untranslated region of mRNA encoded by Clorf24, corresponding to a HOST TARGET binding site such as BINDING SITE I, BINDING SITE II or BINDING SITE III. Table 2 illustrates the complementarity of the nucleotide sequences of Clorf24 BINDING SITE, designated SEQ ID:2351, to the nucleotide sequence of VGAM242 RNA, herein designated VGAM RNA, also designated SEQ ID:577.

Another function of VGAM242 is therefore inhibition of Chromosome 1 Open Reading Frame 24 (Clorf24, Accession NM_{—}052966). Accordingly, utilities of VGAM242 include diagnosis, prevention and treatment of diseases and clinical conditions associated with Clorf24. Chromosome 21 Open Reading Frame 67 (C21orf67, Accession NM_{—}058188) is another VGAM242 host target gene. C21orf67 BINDING SITE is HOST TARGET binding site found in the 5′ untranslated region of mRNA encoded by C21orf67, corresponding to a HOST TARGET binding site such as BINDING SITE I, BINDING SITE II or BINDING SITE III. Table 2 illustrates the complementarity of the nucleotide sequences of C21orf67 BINDING SITE, designated SEQ ID:2367, to the nucleotide sequence of VGAM242 RNA, herein designated VGAM RNA, also designated SEQ ID:577.

Another function of VGAM242 is therefore inhibition of Chromosome 21 Open Reading Frame 67 (C21orf67, Accession NM_{—}058188). Accordingly, utilities of VGAM242 include diagnosis, prevention and treatment of diseases and clinical conditions associated with C21orf67. KIAA0940 (Accession NM_{—}014912) is another VGAM242 host target gene. KIAA0940 BINDING SITE is HOST TARGET binding site found in the 3′ untranslated region of mRNA encoded by KIAA0940, corresponding to a HOST TARGET binding site such as BINDING SITE I, BINDING SITE II or BINDING SITE III. Table 2 illustrates the complementarity of the nucleotide sequences of KIAA0940 BINDING SITE, designated SEQ ID:1585, to the nucleotide sequence of VGAM242 RNA, herein designated VGAM RNA, also designated SEQ ID:577.

Another function of VGAM242 is therefore inhibition of KIAA0940 (Accession NM_{—}014912). Accordingly, utilities of VGAM242 include diagnosis, prevention and treatment of diseases and clinical conditions associated with KIAA0940. LOC158722 (Accession XM_{—}088653) is another VGAM242 host target gene. LOC158722 BINDING SITE is HOST TARGET binding site found in the 3′ untranslated region of mRNA encoded by LOC158722, corresponding to a HOST TARGET binding site such as BINDING SITE I, BINDING SITE II or BINDING SITE III. Table 2 illustrates the complementarity of the nucleotide sequences of LOC158722 BINDING SITE, designated SEQ ID:3225, to the nucleotide sequence of VGAM242 RNA, herein designated VGAM RNA, also designated SEQ ID:577.

Another function of VGAM242 is therefore inhibition of LOC158722 (Accession XM_{—}088653). Accordingly, utilities of VGAM242 include diagnosis, prevention and treatment of diseases and clinical conditions associated with LOC158722. LOC220705 (Accession XM_{—}166000) is another VGAM242 host target gene. LOC220705 BINDING SITE is HOST TARGET binding site found in the 3′ untranslated region of mRNA encoded by LOC220705, corresponding to a HOST TARGET binding site such as BINDING SITE I, BINDING SITE II or BINDING SITE III. Table 2 illustrates the complementarity of the nucleotide sequences of LOC220705 BINDING SITE, designated SEQ ID:3514, to the nucleotide sequence of VGAM242 RNA, herein designated VGAM RNA, also designated SEQ ID:577.

Another function of VGAM242 is therefore inhibition of LOC220705 (Accession XM_{—}166000). Accordingly, utilities of VGAM242 include diagnosis, prevention and treatment of diseases and clinical conditions associated with LOC220705. FIG. 1 further provides a conceptual description of a novel bioinformatically detected viral gene of the present invention, referred to here as Viral Genomic Address Messenger 243 (VGAM243) viral gene, which modulates expression of respective host target genes thereof, the function and utility of which host target genes is known in the art.

VGAM243 is a novel bioinformatically detected regulatory, non protein coding, viral micro RNA (miRNA) gene. The method by which VGAM243 was detected is described hereinabove with reference to FIGS. 1-8.

VGAM243 gene, herein designated VGAM GENE, is a viral gene contained in the genome of Vaccinia Virus. VGAM243 host target gene, herein designated VGAM HOST TARGET GENE, is a human gene contained in the human genome.

VGAM243 gene encodes a VGAM243 precursor RNA, herein designated VGAM PRECURSOR RNA. Similar to other miRNA genes, and unlike most ordinary genes, VGAM243 precursor RNA does not encode a protein. A nucleotide sequence identical or highly similar to the nucleotide sequence of VGAM243 precursor RNA is designated SEQ ID:229, and is provided hereinbelow with reference to the sequence listing part. Nucleotide sequence SEQ ID:229 is located at position 135663 relative to the genome of Vaccinia Virus.

VGAM243 precursor RNA folds onto itself, forming VGAM243 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM243 folded precursor RNA into VGAM243 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM243 RNA is designated SEQ ID:578, and is provided hereinbelow with reference to the sequence listing part.

VGAM243 host target gene, herein designated VGAM HOST TARGET GENE, encodes a corresponding messenger RNA, VGAM243 host target RNA, herein designated VGAM HOST TARGET RNA. VGAM243 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM243 RNA, herein designated VGAM RNA, binds complementarily to one or more host target binding sites located in untranslated regions of VGAM243 host target RNA, herein designated VGAM HOST TARGET RNA. This complementary binding is due to the fact that the nucleotide sequence of VGAM243 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM243 RNA, herein designated VGAM RNA, may have a different number of host target binding sites in untranslated regions of a VGAM243 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM243 RNA, herein designated VGAM RNA, to host target binding sites on VGAM243 host target RNA, herein designated VGAM HOST TARGET RNA, such as BINDING SITE I, BINDING SITE II and BINDING SITE III, inhibits translation of VGAM243 host target RNA into VGAM243 host target protein, herein designated VGAM HOST TARGET PROTEIN. VGAM host target protein is therefore outlined by a broken line.

It is appreciated that VGAM243 host target gene, herein designated VGAM HOST TARGET GENE, in fact represents a plurality of VGAM243 host target genes. The mRNA of each one of this plurality of VGAM243 host target genes comprises one or more host target binding sites, each having a nucleotide sequence which is at least partly complementary to VGAM243 RNA, herein designated VGAM RNA, and which when bound by VGAM243 RNA causes inhibition of translation of respective one or more VGAM243 host target proteins.

It is further appreciated by one skilled in the art that the mode of translational inhibition illustrated by FIG. 1 with specific reference to translational inhibition exerted by VGAM243 gene, herein designated VGAM GENE, on one or more VGAM243 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM243 is inhibition of expression of host target genes, as part of a novel viral mechanism of attacking a host. Accordingly, utilities of VGAM243 include diagnosis, prevention and treatment of viral infection by Vaccinia Virus. Specific functions, and accordingly utilities, of VGAM243 correlate with, and may be deduced from, the identity of the host target genes which VGAM243 binds and inhibits, and the function of these host target genes, as elaborated hereinbelow.

Nucleotide sequences of the VGAM243 precursor RNA, herein designated VGAM PRECURSOR RNA, and of the ‘diced’ VGAM243 RNA, herein designated VGAM RNA, and a schematic representation of the secondary folding of VGAM243 folded precursor RNA, herein designated VGAM FOLDED PRECURSOR RNA, of VGAM243 are further described hereinbelow with reference to Table 1.

Nucleotide sequences of host target binding sites, such as BINDING SITE-I, BINDING SITE-II and BINDING SITE-III of FIG. 1, found on VGAM243 host target RNA, and schematic representation of the complementarity of each of these host target binding sites to VGAM243 RNA, herein designated VGAM RNA, are described hereinbelow with reference to Table 2.

As mentioned hereinabove with reference to FIG. 1, a function of VGAM243 gene, herein designated VGAM is inhibition of expression of VGAM243 target genes. It is appreciated that specific functions, and accordingly utilities, of VGAM243 correlate with, and may be deduced from, the identity of the target genes which VGAM243 binds and inhibits, and the function of these target genes, as elaborated hereinbelow.

Activin A Receptor, Type I (ACVR1, Accession NM_{—}001105) is a VGAM243 host target gene. ACVR1 BINDING SITE is HOST TARGET binding site found in the 3′ untranslated region of mRNA encoded by ACVR1, corresponding to a HOST TARGET binding site such as BINDING SITE I, BINDING SITE II or BINDING SITE III. Table 2 illustrates the complementarity of the nucleotide sequences of ACVR1 BINDING SITE, designated SEQ ID:800, to the nucleotide sequence of VGAM243 RNA, herein designated VGAM RNA, also designated SEQ ID:578.

A function of VGAM243 is therefore inhibition of Activin A Receptor, Type I (ACVR1, Accession NM_{—}001105), a gene which Activin receptor-like kinase; similar to activin, TGF-beta, and C. elegans daf-1 receptors. Accordingly, utilities of VGAM243 include diagnosis, prevention and treatment of diseases and clinical conditions associated with ACVR1. The function of ACVR1 has been established by previous studies. See ACVRLK1 (OMIM Ref. No. 601284). Although activins were discovered by virtue of their capacity to stimulate the production of follicle-stimulating hormone (FSH; 136530) by the pituitary gland and inhibins were initially characterized as FSH inhibitors, activins and inhibins are dimeric proteins that share a common subunit. There are 3 activins (A, B, and A-B), comprising different combinations of 2 closely related beta subunits (beta-A/beta-A; beta-B/beta-B; and beta-A/beta-B, respectively) and 2 inhibins (A and B), consisting of 1 beta-subunit and an inhibin-specific alpha subunit (alpha/beta-A and alpha/beta-B). Activins impinge on a much broader spectrum of cells than do inhibins; however, in those systems in which both proteins are functional, they have opposing biologic effects. Activins are members of a family of polypeptide growth factors that includes also the transforming growth factors-beta (190180, 190220, 190230), mullerian duct-inhibiting substance, and several bone morphogenetic proteins. Human cDNA clones encoding 4 putative transmembrane ser/thr kinases were identified by ten Dijke et al. (1993). By Southern blot analysis of DNAs from a somatic cell hybrid mapping panel, Roijer et al. (1998) mapped the ACVR1 gene to chromosome 2. By fluorescence in situ hybridization, they regionalized the gene to 2q23-q24.

Full details of the abovementioned studies are described in the following publications, the disclosure of which are hereby incorporated by reference:
ten Dijke, P.; Ichijo, H.; Franzen, P.; Schulz, P.; Saras, J.; Toyoshima, H.; Heldin, C.-H.; Miyazono, K.: Activin receptor-like kinases: a novel subclass of cell-surface receptors with predicted serine/threonine kinase activity. Oncogene 8: 2879-2887, 1993.; and
Roijer, E.; Miyazono, K.; Astrom, A.-K.; Geurts van Kessel, A.; ten Dijke, P.; Stenman, G.: Chromosomal localization of three human genes encoding members of the TGF-beta super family of.

Further studies establishing the function and utilities of ACVR1 are found in John Hopkins OMIM database record ID 102576, and in sited publications numbered 944-948 listed in the bibliography section hereinbelow, which are also hereby incorporated by reference. Cadherin, EGF LAG Seven-pass G-type Receptor 1 (flamingo homolog, Drosophila) (CELSR1, Accession NM_{—}014246) is another VGAM243 host target gene. CELSR1 BINDING SITE is HOST TARGET binding site found in the 3′ untranslated region of mRNA encoded by CELSR1, corresponding to a HOST TARGET binding site such as BINDING SITE I, BINDING SITE II or BINDING SITE III. Table 2 illustrates the complementarity of the nucleotide sequences of CELSR1 BINDING SITE, designated SEQ ID: 1486, to the nucleotide sequence of VGAM243 RNA, herein designated VGAM RNA, also designated SEQ ID:578.

Another function of VGAM243 is therefore inhibition of Cadherin, EGF LAG Seven-pass G-type Receptor 1 (flamingo homolog, Drosophila) (CELSR1, Accession NM_{—}014246), a gene which is involved in contact-mediated communication. Accordingly, utilities of VGAM243 include diagnosis, prevention and treatment of diseases and clinical conditions associated with CELSR1. The function of CELSR1 has been established by previous studies. By screening a mouse embryonic cDNA library, Hadjantonakis et al. (1997, 1998) obtained a cDNA encoding a 3,034-amino acid 7-pass transmembrane G protein-coupled receptor, which they termed cadherin EGF LAG seven-pass G-type receptor-1 (OMIM Ref. No. Celsr1). Celsr1 contains motifs that are recognized as mediators of protein-protein interactions. In its extracellular region it has a block of contiguous cadherin repeats in the N terminus and then a region with 7 epidermal growth factor (EGF; 131530)-like repeats interrupted by 2 laminin A (OMIM Ref. No. 150320) G-type (LAG) repeats. By in situ hybridization and RT-PCR analysis, Hadjantonakis et al. (1997) detected significant levels of Celsr1 in neural tube, brain, lung epithelium, and nascent eyelid in day 11.5 mouse embryos. In adult mice, expression was detected in spinal cord, eye, and brain, chiefly in ependymal cells lining the lateral, third, and fourth ventricles. The structure, putative G-linked signaling properties, and restricted expression of the Celsr1 protein suggest that it is a receptor involved in contact-mediated communication.

Full details of the abovementioned studies are described in the following publications, the disclosure of which are hereby incorporated by reference:
Hadjantonakis, A.-K.; Formstone, C. J.; Little, P. F. R.: mCelsr1 is an evolutionarily conserved seven-pass transmembrane receptor and is expressed during mouse embryonic development. Mech. Dev. 78: 91-95, 1998.; and
Hadjantonakis, A.-K.; Sheward, W. J.; Harmar, A. J.; de Galan, L.; Hoovers, J. M. N.; Little, P. F. R.: Celsr1, a neural-specific gene encoding an unusual seven-pass transmembrane rece.

Further studies establishing the function and utilities of CELSR1 are found in John Hopkins OMIM database record ID 604523, and in sited publications numbered 1600-160 and 1673 listed in the bibliography section hereinbelow, which are also hereby incorporated by reference. Gap Junction Protein, Alpha 1, 43 kDa (connexin 43) (GJA1, Accession NM_{—}000165) is another VGAM243 host target gene. GJA1 BINDING SITE is HOST TARGET binding site found in the 3′ untranslated region of mRNA encoded by GJA1, corresponding to a HOST TARGET binding site such as BINDING SITE I, BINDING SITE II or BINDING SITE III. Table 2 illustrates the complementarity of the nucleotide sequences of GJA1 BINDING SITE, designated SEQ ID:715, to the nucleotide sequence of VGAM243 RNA, herein designated VGAM RNA, also designated SEQ ID:578.

Another function of VGAM243 is therefore inhibition of Gap Junction Protein, Alpha 1, 43 kDa (connexin 43) (GJA1, Accession NM_{—}000165), a gene which may act in synchronizing heart contraction and embryonic development. Accordingly, utilities of VGAM243 include diagnosis, prevention and treatment of diseases and clinical conditions associated with GJA1. The function of GJA1 has been established by previous studies. The migration of lymphocytes from the circulation into tissues involves a number of adhesion molecules and the expression of new molecules. Gap junctions facilitate cell-to-cell adhesion and provide pathways for direct intercellular communication. Oviedo-Orta et al. (2000) noted that GJA1 is expressed in a number of lymphoid organs. By RT-PCR, Western blot, and flow cytometric analyses, they showed that lymphocytes express GJA1 and GJA5 (OMIM Ref. No. 121013), but not GJB2 (OMIM Ref. No. 121011), GJB1 (OMIM Ref. No. 304040), GJA4 (OMIM Ref. No. 121012), or GJA7; GJA5 expression was restricted to tonsillar T and B lymphocytes. Flow cytometric analysis showed that GJA1 and GJA5 expression increases after mitogenic stimulation. Extracellular connexin mimetic peptide blocked dye transfer between lymphocyte subpopulations, and gap junction inhibitors decreased the production of IgM in cocultured T and B lymphocytes. The results identified gap junction proteins as important cell surface components that modulate immune responses. Animal model experiments lend further support to the function of GJA1. By targeted mutagenesis of connexin-43, Reaume et al. (1995) showed that its absence was compatible with survival of mouse embryos to term, even though cell lines mutant in Cx43 showed reduced dye coupling in vitro as assessed by injection of carboxyfluorescein. The latter test indicated a reduction, but not complete absence, of junctional communication. However, mutant embryos died at birth as a result of a failure in pulmonary gas exchange caused by a swelling and blockage of the right ventricular outflow tract from the heart. Reaume et al. (1995) interpreted this finding as indicating that Cx43 plays an essential role in heart development but that there is functional compensation among connexins in other parts of the developing fetus.

It is appreciated that the abovementioned animal model for GJA1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Oviedo-Orta, E.; Hoy, T.; Evans, W. H.: Intercellular communication in the immune system: differential expression of connexin 40 and 43, and perturbation of gap junction channel functions in peripheral blood and tonsil human lymphocyte subpopulations. Immunology 99: 578-590, 2000.; and
Reaume, A. G.; de Sousa, P. A.; Kulkarni, S.; Langille, B. L.; Zhu, D.; Davies, T. C.; Juneja, S. C.; Kidder, G. M.; Rossant, J.: Cardiac malformation in neonatal mice lacking connex.

Further studies establishing the function and utilities of GJA1 are found in John Hopkins OMIM database record ID 121014, and in sited publications numbered 2736-2741, 2788, 18-21, 2818-24, 2735-27, 27-2 and 2734 listed in the bibliography section hereinbelow, which are also hereby incorporated by reference. Phospholamban (PLN, Accession NM_{—}002667) is another VGAM243 host target gene. PLN BINDING SITE is HOST TARGET binding site found in the 3′ untranslated region of mRNA encoded by PLN, corresponding to a HOST TARGET binding site such as BINDING SITE I, BINDING SITE II or BINDING SITE III. Table 2 illustrates the complementarity of the nucleotide sequences of PLN BINDING SITE, designated SEQ ID:945, to the nucleotide sequence of VGAM243 RNA, herein designated VGAM RNA, also designated SEQ ID:578.

Another function of VGAM243 is therefore inhibition of Phospholamban (PLN, Accession NM_{—}002667), a gene which regulates the activity of the calcium pump of cardiac sarcoplasmic reticulum. Accordingly, utilities of VGAM243 include diagnosis, prevention and treatment of diseases and clinical conditions associated with PLN. The function of PLN and its association with various diseases and clinical conditions, has been established by previous studies, as described hereinabove with reference to VGAM104. CCR4—NOT Transcription Complex, Subunit 7 (CNOT7, Accession NM_{—}054026) is another VGAM243 host target gene. CNOT7 BINDING SITE is HOST TARGET binding site found in the 3′ untranslated region of mRNA encoded by CNOT7, corresponding to a HOST TARGET binding site such as BINDING SITE I, BINDING SITE II or BINDING SITE III. Table 2 illustrates the complementarity of the nucleotide sequences of CNOT7 BINDING SITE, designated SEQ ID:2363, to the nucleotide sequence of VGAM243 RNA, herein designated VGAM RNA, also designated SEQ ID:578.

Another function of VGAM243 is therefore inhibition of CCR4—NOT Transcription Complex, Subunit 7 (CNOT7, Accession NM_{—}054026). Accordingly, utilities of VGAM243 include diagnosis, prevention and treatment of diseases and clinical conditions associated with CNOT7. Grcc9 (Accession NM_{—}032641) is another VGAM243 host target gene. Grcc9 BINDING SITE is HOST TARGET binding site found in the 3′ untranslated region of mRNA encoded by Grcc9, corresponding to a HOST TARGET binding site such as BINDING SITE I, BINDING SITE II or BINDING SITE III. Table 2 illustrates the complementarity of the nucleotide sequences of Grcc9 BINDING SITE, designated SEQ ID:2268, to the nucleotide sequence of VGAM243 RNA, herein designated VGAM RNA, also designated SEQ ID:578.

Another function of VGAM243 is therefore inhibition of Grcc9 (Accession NM_{—}032641). Accordingly, utilities of VGAM243 include diagnosis, prevention and treatment of diseases and clinical conditions associated with Grcc9. KIAA1287 (Accession XM_{—}085753) is another VGAM243 host target gene. KIAA1287 BINDING SITE is HOST TARGET binding site found in the 3′ untranslated region of mRNA encoded by KIAA1287, corresponding to a HOST TARGET binding site such as BINDING SITE I, BINDING SITE II or BINDING SITE III. Table 2 illustrates the complementarity of the nucleotide sequences of KIAA1287 BINDING SITE, designated SEQ ID:3106, to the nucleotide sequence of VGAM243 RNA, herein designated VGAM RNA, also designated SEQ ID:578.

Another function of VGAM243 is therefore inhibition of KIAA1287 (Accession XM_{—}085753). Accordingly, utilities of VGAM243 include diagnosis, prevention and treatment of diseases and clinical conditions associated with KIAA1287. KIAA1673 (Accession XM_{—}047672) is another VGAM243 host target gene. KIAA1673 BINDING SITE is HOST TARGET binding site found in the 3′ untranslated region of mRNA encoded by KIAA1673, corresponding to a HOST TARGET binding site such as BINDING SITE I, BINDING SITE II or BINDING SITE III. Table 2 illustrates the complementarity of the nucleotide sequences of KIAA1673 BINDING SITE, designated SEQ ID:2894, to the nucleotide sequence of VGAM243 RNA, herein designated VGAM RNA, also designated SEQ ID:578.

Another function of VGAM243 is therefore inhibition of KIAA1673 (Accession XM_{—}047672). Accordingly, utilities of VGAM243 include diagnosis, prevention and treatment of diseases and clinical conditions associated with KIAA1673. Lipoma HMGIC Fusion Partner (LHFP, Accession NM_{—}005780) is another VGAM243 host target gene. LHFP BINDING SITE is HOST TARGET binding site found in the 3′ untranslated region of mRNA encoded by LHFP, corresponding to a HOST TARGET binding site such as BINDING SITE I, BINDING SITE II or BINDING SITE III. Table 2 illustrates the complementarity of the nucleotide sequences of LHFP BINDING SITE, designated SEQ ID:1248, to the nucleotide sequence of VGAM243 RNA, herein designated VGAM RNA, also designated SEQ ID:578.

Another function of VGAM243 is therefore inhibition of Lipoma HMGIC Fusion Partner (LHFP, Accession NM_{—}005780). Accordingly, utilities of VGAM243 include diagnosis, prevention and treatment of diseases and clinical conditions associated with LHFP. Phosphodiesterase 10A (PDE10A, Accession NM_{—}006661) is another VGAM243 host target gene. PDE10A BINDING SITE is HOST TARGET binding site found in the 3′ untranslated region of mRNA encoded by PDE10A, corresponding to a HOST TARGET binding site such as BINDING SITE I, BINDING SITE II or BINDING SITE III. Table 2 illustrates the complementarity of the nucleotide sequences of PDE10A BINDING SITE, designated SEQ ID:1320, to the nucleotide sequence of VGAM243 RNA, herein designated VGAM RNA, also designated SEQ ID:578.

Another function of VGAM243 is therefore inhibition of Phosphodiesterase 10A (PDE10A, Accession NM_{—}006661). Accordingly, utilities of VGAM243 include diagnosis, prevention and treatment of diseases and clinical conditions associated with PDE10A. TBDN100 (Accession NM_{—}025085) is another VGAM243 host target gene. TBDN100 BINDING SITE is HOST TARGET binding site found in the 3′ untranslated region of mRNA encoded by TBDN100, corresponding to a HOST TARGET binding site such as BINDING SITE I, BINDING SITE II or BINDING SITE III. Table 2 illustrates the complementarity of the nucleotide sequences of TBDN100 BINDING SITE, designated SEQ ID:2135, to the nucleotide sequence of VGAM243 RNA, herein designated VGAM RNA, also designated SEQ ID:578.

Another function of VGAM243 is therefore inhibition of TBDN100 (Accession NM_{—}025085). Accordingly, utilities of VGAM243 include diagnosis, prevention and treatment of diseases and clinical conditions associated with TBDN100. LOC149448 (Accession XM_{—}097642) is another VGAM243 host target gene. LOC149448 BINDING SITE is HOST TARGET binding site found in the 3′ untranslated region of mRNA encoded by LOC149448, corresponding to a HOST TARGET binding site such as BINDING SITE I, BINDING SITE II or BINDING SITE III. Table 2 illustrates the complementarity of the nucleotide sequences of LOC149448 BINDING SITE, designated SEQ ID:3310, to the nucleotide sequence of VGAM243 RNA, herein designated VGAM RNA, also designated SEQ ID:578.

Another function of VGAM243 is therefore inhibition of LOC149448 (Accession XM_{—}097642). Accordingly, utilities of VGAM243 include diagnosis, prevention and treatment of diseases and clinical conditions associated with LOC149448. LOC155434 (Accession XM_{—}098723) is another VGAM243 host target gene. LOC155434 BINDING SITE is HOST TARGET binding site found in the 3′ untranslated region of mRNA encoded by LOC155434, corresponding to a HOST TARGET binding site such as BINDING SITE I, BINDING SITE II or BINDING SITE III. Table 2 illustrates the complementarity of the nucleotide sequences of LOC155434 BINDING SITE, designated SEQ ID:3375, to the nucleotide sequence of VGAM243 RNA, herein designated VGAM RNA, also designated SEQ ID:578.

Another function of VGAM243 is therefore inhibition of LOC155434 (Accession XM_{—}098723). Accordingly, utilities of VGAM243 include diagnosis, prevention and treatment of diseases and clinical conditions associated with LOC155434. LOC221583 (Accession XM_{—}166396) is another VGAM243 host target gene. LOC221583 BINDING SITE is HOST TARGET binding site found in the 3′ untranslated region of mRNA encoded by LOC221583, corresponding to a HOST TARGET binding site such as BINDING SITE I, BINDING SITE II or BINDING SITE III. Table 2 illustrates the complementarity of the nucleotide sequences of LOC221583 BINDING SITE, designated SEQ ID:3557, to the nucleotide sequence of VGAM243 RNA, herein designated VGAM RNA, also designated SEQ ID:578.

Another function of VGAM243 is therefore inhibition of LOC221583 (Accession XM_{—}166396). Accordingly, utilities of VGAM243 include diagnosis, prevention and treatment of diseases and clinical conditions associated with LOC221583. LOC54466 (Accession NM_{—}019003) is another VGAM243 host target gene. LOC54466 BINDING SITE is HOST TARGET binding site found in the 3′ untranslated region of mRNA encoded by LOC54466, corresponding to a HOST TARGET binding site such as BINDING SITE I, BINDING SITE II or BINDING SITE III. Table 2 illustrates the complementarity of the nucleotide sequences of LOC54466 BINDING SITE, designated SEQ ID:1871, to the nucleotide sequence of VGAM243 RNA, herein designated VGAM RNA, also designated SEQ ID:578.

Another function of VGAM243 is therefore inhibition of LOC54466 (Accession NM_{—}019003). Accordingly, utilities of VGAM243 include diagnosis, prevention and treatment of diseases and clinical conditions associated with LOC54466. FIG. 1 further provides a conceptual description of a novel bioinformatically detected viral gene of the present invention, referred to here as Viral Genomic Address Messenger 244 (VGAM244) viral gene, which modulates expression of respective host target genes thereof, the function and utility of which host target genes is known in the art.

VGAM244 is a novel bioinformatically detected regulatory, non protein coding, viral micro RNA (miRNA) gene. The method by which VGAM244 was detected is described hereinabove with reference to FIGS. 1-8.

VGAM244 gene, herein designated VGAM GENE, is a viral gene contained in the genome of Vaccinia Virus. VGAM244 host target gene, herein designated VGAM HOST TARGET GENE, is a human gene contained in the human genome.

VGAM244 gene encodes a VGAM244 precursor RNA, herein designated VGAM PRECURSOR RNA. Similar to other miRNA genes, and unlike most ordinary genes, VGAM244 precursor RNA does not encode a protein. A nucleotide sequence identical or highly similar to the nucleotide sequence of VGAM244 precursor RNA is designated SEQ ID:230, and is provided hereinbelow with reference to the sequence listing part. Nucleotide sequence SEQ ID:230 is located at position 134932 relative to the genome of Vaccinia Virus.

VGAM244 precursor RNA folds onto itself, forming VGAM244 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM244 folded precursor RNA into VGAM244 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM244 RNA is designated SEQ ID:579, and is provided hereinbelow with reference to the sequence listing part.

VGAM244 host target gene, herein designated VGAM HOST TARGET GENE, encodes a corresponding messenger RNA, VGAM244 host target RNA, herein designated VGAM HOST TARGET RNA. VGAM244 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM244 RNA, herein designated VGAM RNA, binds complementarily to one or more host target binding sites located in untranslated regions of VGAM244 host target RNA, herein designated VGAM HOST TARGET RNA. This complementary binding is due to the fact that the nucleotide sequence of VGAM244 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM244 RNA, herein designated VGAM RNA, may have a different number of host target binding sites in untranslated regions of a VGAM244 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM244 RNA, herein designated VGAM RNA, to host target binding sites on VGAM244 host target RNA, herein designated VGAM HOST TARGET RNA, such as BINDING SITE I, BINDING SITE II and BINDING SITE III, inhibits translation of VGAM244 host target RNA into VGAM244 host target protein, herein designated VGAM HOST TARGET PROTEIN. VGAM host target protein is therefore outlined by a broken line.

It is appreciated that VGAM244 host target gene, herein designated VGAM HOST TARGET GENE, in fact represents a plurality of VGAM244 host target genes. The mRNA of each one of this plurality of VGAM244 host target genes comprises one or more host target binding sites, each having a nucleotide sequence which is at least partly complementary to VGAM244 RNA, herein designated VGAM RNA, and which when bound by VGAM244 RNA causes inhibition of translation of respective one or more VGAM244 host target proteins.

It is further appreciated by one skilled in the art that the mode of translational inhibition illustrated by FIG. 1 with specific reference to translational inhibition exerted by VGAM244 gene, herein designated VGAM GENE, on one or more VGAM244 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM244 is inhibition of expression of host target genes, as part of a novel viral mechanism of attacking a host. Accordingly, utilities of VGAM244 include diagnosis, prevention and treatment of viral infection by Vaccinia Virus. Specific functions, and accordingly utilities, of VGAM244 correlate with, and may be deduced from, the identity of the host target genes which VGAM244 binds and inhibits, and the function of these host target genes, as elaborated hereinbelow.

Nucleotide sequences of the VGAM244 precursor RNA, herein designated VGAM PRECURSOR RNA, and of the ‘diced’ VGAM244 RNA, herein designated VGAM RNA, and a schematic representation of the secondary folding of VGAM244 folded precursor RNA, herein designated VGAM FOLDED PRECURSOR RNA, of VGAM244 are further described hereinbelow with reference to Table 1.

Nucleotide sequences of host target binding sites, such as BINDING SITE-I, BINDING SITE-II and BINDING SITE-III of FIG. 1, found on VGAM244 host target RNA, and schematic representation of the complementarity of each of these host target binding sites to VGAM244 RNA, herein designated VGAM RNA, are described hereinbelow with reference to Table 2.

As mentioned hereinabove with reference to FIG. 1, a function of VGAM244 gene, herein designated VGAM is inhibition of expression of VGAM244 target genes. It is appreciated that specific functions, and accordingly utilities, of VGAM244 correlate with, and may be deduced from, the identity of the target genes which VGAM244 binds and inhibits, and the function of these target genes, as elaborated hereinbelow.

Methyl CpG Binding Protein 2 (Rett syndrome) (MECP2, Accession NM_{—}004992) is a VGAM244 host target gene. MECP2 BINDING SITE is HOST TARGET binding site found in the 3′ untranslated region of mRNA encoded by MECP2, corresponding to a HOST TARGET binding site such as BINDING SITE I, BINDING SITE II or BINDING SITE III. Table 2 illustrates the complementarity of the nucleotide sequences of MECP2 BINDING SITE, designated SEQ ID:1167, to the nucleotide sequence of VGAM244 RNA, herein designated VGAM RNA, also designated SEQ ID:579.

A function of VGAM244 is therefore inhibition of Methyl CpG Binding Protein 2 (Rett syndrome) (MECP2, Accession NM_{—}004992). Accordingly, utilities of VGAM244 include diagnosis, prevention and treatment of diseases and clinical conditions associated with MECP2. ATPase, Class V, Type 10B (ATP10B, Accession XM_{—}032721) is another VGAM244 host target gene. ATP10B BINDING SITE is HOST TARGET binding site found in the 3′ untranslated region of mRNA encoded by ATP10B, corresponding to a HOST TARGET binding site such as BINDING SITE I, BINDING SITE II or BINDING SITE III. Table 2 illustrates the complementarity of the nucleotide sequences of ATP10B BINDING SITE, designated SEQ ID:2655, to the nucleotide sequence of VGAM244 RNA, herein designated VGAM RNA, also designated SEQ ID:579.

Another function of VGAM244 is therefore inhibition of ATPase, Class V, Type 10B (ATP10B, Accession XM_{—}032721). Accordingly, utilities of VGAM244 include diagnosis, prevention and treatment of diseases and clinical conditions associated with ATP10B. BDG-29 (Accession XM_{—}051343) is another VGAM244 host target gene. BDG-29 BINDING SITE is HOST TARGET binding site found in the 3′ untranslated region of mRNA encoded by BDG-29, corresponding to a HOST TARGET binding site such as BINDING SITE I, BINDING SITE II or BINDING SITE III. Table 2 illustrates the complementarity of the nucleotide sequences of BDG-29 BINDING SITE, designated SEQ ID:2943, to the nucleotide sequence of VGAM244 RNA, herein designated VGAM RNA, also designated SEQ ID:579.

Another function of VGAM244 is therefore inhibition of BDG-29 (Accession XM_{—}051343). Accordingly, utilities of VGAM244 include diagnosis, prevention and treatment of diseases and clinical conditions associated with BDG-29. GW112 (Accession NM_{—}006418) is another VGAM244 host target gene. GW112 BINDING SITE is HOST TARGET binding site found in the 3′ untranslated region of mRNA encoded by GW112, corresponding to a HOST TARGET binding site such as BINDING SITE I, BINDING SITE II or BINDING SITE III. Table 2 illustrates the complementarity of the nucleotide sequences of GW112 BINDING SITE, designated SEQ ID: 1298, to the nucleotide sequence of VGAM244 RNA, herein designated VGAM RNA, also designated SEQ ID:579.

Another function of VGAM244 is therefore inhibition of GW112 (Accession NM_{—}006418). Accordingly, utilities of VGAM244 include diagnosis, prevention and treatment of diseases and clinical conditions associated with GW112. Heat Shock 27 kDa Protein Family, Member 7 (cardiovascular) (HSPB7, Accession NM_{—}014424) is another VGAM244 host target gene. HSPB7 BINDING SITE is HOST TARGET binding site found in the 3′ untranslated region of mRNA encoded by HSPB7, corresponding to a HOST TARGET binding site such as BINDING SITE I, BINDING SITE II or BINDING SITE III. Table 2 illustrates the complementarity of the nucleotide sequences of HSPB7 BINDING SITE, designated SEQ ID: 1498, to the nucleotide sequence of VGAM244 RNA, herein designated VGAM RNA, also designated SEQ ID:579.

Another function of VGAM244 is therefore inhibition of Heat Shock 27 kDa Protein Family, Member 7 (cardiovascular) (HSPB7, Accession NM_{—}014424). Accordingly, utilities of VGAM244 include diagnosis, prevention and treatment of diseases and clinical conditions associated with HSPB7. KIAA1464 (Accession XM_{—}043069) is another VGAM244 host target gene. KIAA1464 BINDING SITE is HOST TARGET binding site found in the 3′ untranslated region of mRNA encoded by KIAA1464, corresponding to a HOST TARGET binding site such as BINDING SITE I, BINDING SITE II or BINDING SITE III. Table 2 illustrates the complementarity of the nucleotide sequences of KIAA1464 BINDING SITE, designated SEQ ID:2813, to the nucleotide sequence of VGAM244 RNA, herein designated VGAM RNA, also designated SEQ ID:579.

Another function of VGAM244 is therefore inhibition of KIAA1464 (Accession XM_{—}043069). Accordingly, utilities of VGAM244 include diagnosis, prevention and treatment of diseases and clinical conditions associated with KIAA1464. KIAA1634 (Accession XM_{—}032749) is another VGAM244 host target gene. KIAA1634 BINDING SITE is HOST TARGET binding site found in the 3′ untranslated region of mRNA encoded by KIAA1634, corresponding to a HOST TARGET binding site such as BINDING SITE I, BINDING SITE II or BINDING SITE III. Table 2 illustrates the complementarity of the nucleotide sequences of KIAA1634 BINDING SITE, designated SEQ ID:2656, to the nucleotide sequence of VGAM244 RNA, herein designated VGAM RNA, also designated SEQ ID:579.

Another function of VGAM244 is therefore inhibition of KIAA1634 (Accession XM_{—}032749). Accordingly, utilities of VGAM244 include diagnosis, prevention and treatment of diseases and clinical conditions associated with KIAA1634. Neuropilin (NRP) and Tolloid (TLL)-like 1 (NETO1, Accession NM_{—}138999) is another VGAM244 host target gene. NETO1 BINDING SITE is HOST TARGET binding site found in the 3′ untranslated region of mRNA encoded by NETO1, corresponding to a HOST TARGET binding site such as BINDING SITE I, BINDING SITE II or BINDING SITE III. Table 2 illustrates the complementarity of the nucleotide sequences of NETO1 BINDING SITE, designated SEQ ID:2463, to the nucleotide sequence of VGAM244 RNA, herein designated VGAM RNA, also designated SEQ ID:579.

Another function of VGAM244 is therefore inhibition of Neuropilin (NRP) and Tolloid (TLL)-like 1 (NETO1, Accession NM_{—}138999). Accordingly, utilities of VGAM244 include diagnosis, prevention and treatment of diseases and clinical conditions associated with NETO1. PRO2533 (Accession NM_{—}018629) is another VGAM244 host target gene. PRO2533 BINDING SITE is HOST TARGET binding site found in the 3′ untranslated region of mRNA encoded by PRO2533, corresponding to a HOST TARGET binding site such as BINDING SITE I, BINDING SITE II or BINDING SITE III. Table 2 illustrates the complementarity of the nucleotide sequences of PRO2533 BINDING SITE, designated SEQ ID: 1854, to the nucleotide sequence of VGAM244 RNA, herein designated VGAM RNA, also designated SEQ ID:579.

Another function of VGAM244 is therefore inhibition of PRO2533 (Accession NM_{—}018629). Accordingly, utilities of VGAM244 include diagnosis, prevention and treatment of diseases and clinical conditions associated with PRO2533. LOC151201 (Accession XM_{—}098021) is another VGAM244 host target gene. LOC151201 BINDING SITE is HOST TARGET binding site found in the 3′ untranslated region of mRNA encoded by LOC151201, corresponding to a HOST TARGET binding site such as BINDING SITE I, BINDING SITE II or BINDING SITE III. Table 2 illustrates the complementarity of the nucleotide sequences of LOC151201 BINDING SITE, designated SEQ ID:3343, to the nucleotide sequence of VGAM244 RNA, herein designated VGAM RNA, also designated SEQ ID:579.

Another function of VGAM244 is therefore inhibition of LOC151201 (Accession XM_{—}098021). Accordingly, utilities of VGAM244 include diagnosis, prevention and treatment of diseases and clinical conditions associated with LOC151201. FIG. 1 further provides a conceptual description of a novel bioinformatically detected viral gene of the present invention, referred to here as Viral Genomic Address Messenger 245 (VGAM245) viral gene, which modulates expression of respective host target genes thereof, the function and utility of which host target genes is known in the art.

VGAM245 is a novel bioinformatically detected regulatory, non protein coding, viral micro RNA (miRNA) gene. The method by which VGAM245 was detected is described hereinabove with reference to FIGS. 1-8.

VGAM245 gene, herein designated VGAM GENE, is a viral gene contained in the genome of Vaccinia Virus. VGAM245 host target gene, herein designated VGAM HOST TARGET GENE, is a human gene contained in the human genome.

VGAM245 gene encodes a VGAM245 precursor RNA, herein designated VGAM PRECURSOR RNA. Similar to other miRNA genes, and unlike most ordinary genes, VGAM245 precursor RNA does not encode a protein. A nucleotide sequence identical or highly similar to the nucleotide sequence of VGAM245 precursor RNA is designated SEQ ID:231, and is provided hereinbelow with reference to the sequence listing part. Nucleotide sequence SEQ ID:231 is located at position 133924 relative to the genome of Vaccinia Virus.

VGAM245 precursor RNA folds onto itself, forming VGAM245 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM245 folded precursor RNA into VGAM245 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM245 RNA is designated SEQ ID:580, and is provided hereinbelow with reference to the sequence listing part.

VGAM245 host target gene, herein designated VGAM HOST TARGET GENE, encodes a corresponding messenger RNA, VGAM245 host target RNA, herein designated VGAM HOST TARGET RNA. VGAM245 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM245 RNA, herein designated VGAM RNA, binds complementarily to one or more host target binding sites located in untranslated regions of VGAM245 host target RNA, herein designated VGAM HOST TARGET RNA. This complementary binding is due to the fact that the nucleotide sequence of VGAM245 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM245 RNA, herein designated VGAM RNA, may have a different number of host target binding sites in untranslated regions of a VGAM245 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM245 RNA, herein designated VGAM RNA, to host target binding sites on VGAM245 host target RNA, herein designated VGAM HOST TARGET RNA, such as BINDING SITE I, BINDING SITE II and BINDING SITE III, inhibits translation of VGAM245 host target RNA into VGAM245 host target protein, herein designated VGAM HOST TARGET PROTEIN. VGAM host target protein is therefore outlined by a broken line.

It is appreciated that VGAM245 host target gene, herein designated VGAM HOST TARGET GENE, in fact represents a plurality of VGAM245 host target genes. The mRNA of each one of this plurality of VGAM245 host target genes comprises one or more host target binding sites, each having a nucleotide sequence which is at least partly complementary to VGAM245 RNA, herein designated VGAM RNA, and which when bound by VGAM245 RNA causes inhibition of translation of respective one or more VGAM245 host target proteins.

It is further appreciated by one skilled in the art that the mode of translational inhibition illustrated by FIG. 1 with specific reference to translational inhibition exerted by VGAM245 gene, herein designated VGAM GENE, on one or more VGAM245 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM245 is inhibition of expression of host target genes, as part of a novel viral mechanism of attacking a host. Accordingly, utilities of VGAM245 include diagnosis, prevention and treatment of viral infection by Vaccinia Virus. Specific functions, and accordingly utilities, of VGAM245 correlate with, and may be deduced from, the identity of the host target genes which VGAM245 binds and inhibits, and the function of these host target genes, as elaborated hereinbelow.

Nucleotide sequences of the VGAM245 precursor RNA, herein designated VGAM PRECURSOR RNA, and of the ‘diced’ VGAM245 RNA, herein designated VGAM RNA, and a schematic representation of the secondary folding of VGAM245 folded precursor RNA, herein designated VGAM FOLDED PRECURSOR RNA, of VGAM245 are further described hereinbelow with reference to Table 1.

Nucleotide sequences of host target binding sites, such as BINDING SITE-I, BINDING SITE-II and BINDING SITE-III of FIG. 1, found on VGAM245 host target RNA, and schematic representation of the complementarity of each of these host target binding sites to VGAM245 RNA, herein designated VGAM RNA, are described hereinbelow with reference to Table 2.

As mentioned hereinabove with reference to FIG. 1, a function of VGAM245 gene, herein designated VGAM is inhibition of expression of VGAM245 target genes. It is appreciated that specific functions, and accordingly utilities, of VGAM245 correlate with, and may be deduced from, the identity of the target genes which VGAM245 binds and inhibits, and the function of these target genes, as elaborated hereinbelow.

FLJ14124 (Accession NM_{—}024868) is a VGAM245 host target gene. FLJ14124 BINDING SITE is HOST TARGET binding site found in the 3′ untranslated region of mRNA encoded by FLJ14124, corresponding to a HOST TARGET binding site such as BINDING SITE I, BINDING SITE II or BINDING SITE III. Table 2 illustrates the complementarity of the nucleotide sequences of FLJ14124 BINDING SITE, designated SEQ ID:2099, to the nucleotide sequence of VGAM245 RNA, herein designated VGAM RNA, also designated SEQ ID:580.

A function of VGAM245 is therefore inhibition of FLJ14124 (Accession NM_{—}024868). Accordingly, utilities of VGAM245 include diagnosis, prevention and treatment of diseases and clinical conditions associated with FLJ14124. FLJ20039 (Accession NM_{—}017635) is another VGAM245 host target gene. FLJ20039 BINDING SITE is HOST TARGET binding site found in the 3′ untranslated region of mRNA encoded by FLJ20039, corresponding to a HOST TARGET binding site such as BINDING SITE I, BINDING SITE II or BINDING SITE III. Table 2 illustrates the complementarity of the nucleotide sequences of FLJ20039 BINDING SITE, designated SEQ ID:1735, to the nucleotide sequence of VGAM245 RNA, herein designated VGAM RNA, also designated SEQ ID:580.

Another function of VGAM245 is therefore inhibition of FLJ20039 (Accession NM_{—}017635). Accordingly, utilities of VGAM245 include diagnosis, prevention and treatment of diseases and clinical conditions associated with FLJ20039. FLJ21369 (Accession NM_{—}024802) is another VGAM245 host target gene. FLJ21369 BINDING SITE is HOST TARGET binding site found in the 5′ untranslated region of mRNA encoded by FLJ21369, corresponding to a HOST TARGET binding site such as BINDING SITE I, BINDING SITE II or BINDING SITE III. Table 2 illustrates the complementarity of the nucleotide sequences of FLJ21369 BINDING SITE, designated SEQ ID:2090, to the nucleotide sequence of VGAM245 RNA, herein designated VGAM RNA, also designated SEQ ID:580.

Another function of VGAM245 is therefore inhibition of FLJ21369 (Accession NM_{—}024802). Accordingly, utilities of VGAM245 include diagnosis, prevention and treatment of diseases and clinical conditions associated with FLJ21369. KIAA1577 (Accession XM_{—}035299) is another VGAM245 host target gene. KIAA1577 BINDING SITE is HOST TARGET binding site found in the 5′ untranslated region of mRNA encoded by KIAA1577, corresponding to a HOST TARGET binding site such as BINDING SITE I, BINDING SITE II or BINDING SITE III. Table 2 illustrates the complementarity of the nucleotide sequences of KIAA1577 BINDING SITE, designated SEQ ID:2690, to the nucleotide sequence of VGAM245 RNA, herein designated VGAM RNA, also designated SEQ ID:580.

Another function of VGAM245 is therefore inhibition of KIAA1577 (Accession XM_{—}035299). Accordingly, utilities of VGAM245 include diagnosis, prevention and treatment of diseases and clinical conditions associated with KIAA1577. LOC144524 (Accession XM_{—}096624) is another VGAM245 host target gene. LOC144524 BINDING SITE is HOST TARGET binding site found in the 3′ untranslated region of mRNA encoded by LOC144524, corresponding to a HOST TARGET binding site such as BINDING SITE I, BINDING SITE II or BINDING SITE III. Table 2 illustrates the complementarity of the nucleotide sequences of LOC144524 BINDING SITE, designated SEQ ID:3269, to the nucleotide sequence of VGAM245 RNA, herein designated VGAM RNA, also designated SEQ ID:580.

Another function of VGAM245 is therefore inhibition of LOC144524 (Accession XM_{—}096624). Accordingly, utilities of VGAM245 include diagnosis, prevention and treatment of diseases and clinical conditions associated with LOC144524. LOC150170 (Accession XM_{—}086799) is another VGAM245 host target gene. LOC150170 BINDING SITE is HOST TARGET binding site found in the 5′ untranslated region of mRNA encoded by LOC150170, corresponding to a HOST TARGET binding site such as BINDING SITE I, BINDING SITE II or BINDING SITE III. Table 2 illustrates the complementarity of the nucleotide sequences of LOC150170 BINDING SITE, designated SEQ ID:3143, to the nucleotide sequence of VGAM245 RNA, herein designated VGAM RNA, also designated SEQ ID:580.

Another function of VGAM245 is therefore inhibition of LOC150170 (Accession XM_{—}086799). Accordingly, utilities of VGAM245 include diagnosis, prevention and treatment of diseases and clinical conditions associated with LOC150170. LOC150175 (Accession XM_{—}086806) is another VGAM245 host target gene. LOC150175 BINDING SITE is HOST TARGET binding site found in the 5′ untranslated region of mRNA encoded by LOC150175, corresponding to a HOST TARGET binding site such as BINDING SITE I, BINDING SITE II or BINDING SITE III. Table 2 illustrates the complementarity of the nucleotide sequences of LOC150175 BINDING SITE, designated SEQ ID:3144, to the nucleotide sequence of VGAM245 RNA, herein designated VGAM RNA, also designated SEQ ID:580.

Another function of VGAM245 is therefore inhibition of LOC150175 (Accession XM_{—}086806). Accordingly, utilities of VGAM245 include diagnosis, prevention and treatment of diseases and clinical conditions associated with LOC150175. LOC150215 (Accession XM_{—}086813) is another VGAM245 host target gene. LOC150215 BINDING SITE is HOST TARGET binding site found in the 5′ untranslated region of mRNA encoded by LOC150215, corresponding to a HOST TARGET binding site such as BINDING SITE I, BINDING SITE II or BINDING SITE III. Table 2 illustrates the complementarity of the nucleotide sequences of LOC150215 BINDING SITE, designated SEQ ID:3145, to the nucleotide sequence of VGAM245 RNA, herein designated VGAM RNA, also designated SEQ ID:580.

Another function of VGAM245 is therefore inhibition of LOC150215 (Accession XM_{—}086813). Accordingly, utilities of VGAM245 include diagnosis, prevention and treatment of diseases and clinical conditions associated with LOC150215. LOC150218 (Accession XM_{—}086850) is another VGAM245 host target gene. LOC150218 BINDING SITE is HOST TARGET binding site found in the 5′ untranslated region of mRNA encoded by LOC150218, corresponding to a HOST TARGET binding site such as BINDING SITE I, BINDING SITE II or BINDING SITE III. Table 2 illustrates the complementarity of the nucleotide sequences of LOC150218 BINDING SITE, designated SEQ ID:3146, to the nucleotide sequence of VGAM245 RNA, herein designated VGAM RNA, also designated SEQ ID:580.

Another function of VGAM245 is therefore inhibition of LOC150218 (Accession XM_{—}086850). Accordingly, utilities of VGAM245 include diagnosis, prevention and treatment of diseases and clinical conditions associated with LOC150218. FIG. 1 further provides a conceptual description of a novel bioinformatically detected viral gene of the present invention, referred to here as Viral Genomic Address Messenger 246 (VGAM246) viral gene, which modulates expression of respective host target genes thereof, the function and utility of which host target genes is known in the art.

VGAM246 is a novel bioinformatically detected regulatory, non protein coding, viral micro RNA (miRNA) gene. The method by which VGAM246 was detected is described hereinabove with reference to FIGS. 1-8.

VGAM246 gene, herein designated VGAM GENE, is a viral gene contained in the genome of Vaccinia Virus. VGAM246 host target gene, herein designated VGAM HOST TARGET GENE, is a human gene contained in the human genome.

VGAM246 gene encodes a VGAM246 precursor RNA, herein designated VGAM PRECURSOR RNA. Similar to other miRNA genes, and unlike most ordinary genes, VGAM246 precursor RNA does not encode a protein. A nucleotide sequence identical or highly similar to the nucleotide sequence of VGAM246 precursor RNA is designated SEQ ID:232, and is provided hereinbelow with reference to the sequence listing part. Nucleotide sequence SEQ ID:232 is located at position 136295 relative to the genome of Vaccinia Virus.

VGAM246 precursor RNA folds onto itself, forming VGAM246 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM246 folded precursor RNA into VGAM246 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM246 RNA is designated SEQ ID:581, and is provided hereinbelow with reference to the sequence listing part.

VGAM246 host target gene, herein designated VGAM HOST TARGET GENE, encodes a corresponding messenger RNA, VGAM246 host target RNA, herein designated VGAM HOST TARGET RNA. VGAM246 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM246 RNA, herein designated VGAM RNA, binds complementarily to one or more host target binding sites located in untranslated regions of VGAM246 host target RNA, herein designated VGAM HOST TARGET RNA. This complementary binding is due to the fact that the nucleotide sequence of VGAM246 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM246 RNA, herein designated VGAM RNA, may have a different number of host target binding sites in untranslated regions of a VGAM246 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM246 RNA, herein designated VGAM RNA, to host target binding sites on VGAM246 host target RNA, herein designated VGAM HOST TARGET RNA, such as BINDING SITE I, BINDING SITE II and BINDING SITE III, inhibits translation of VGAM246 host target RNA into VGAM246 host target protein, herein designated VGAM HOST TARGET PROTEIN. VGAM host target protein is therefore outlined by a broken line.

It is appreciated that VGAM246 host target gene, herein designated VGAM HOST TARGET GENE, in fact represents a plurality of VGAM246 host target genes. The mRNA of each one of this plurality of VGAM246 host target genes comprises one or more host target binding sites, each having a nucleotide sequence which is at least partly complementary to VGAM246 RNA, herein designated VGAM RNA, and which when bound by VGAM246 RNA causes inhibition of translation of respective one or more VGAM246 host target proteins.

It is further appreciated by one skilled in the art that the mode of translational inhibition illustrated by FIG. 1 with specific reference to translational inhibition exerted by VGAM246 gene, herein designated VGAM GENE, on one or more VGAM246 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM246 is inhibition of expression of host target genes, as part of a novel viral mechanism of attacking a host. Accordingly, utilities of VGAM246 include diagnosis, prevention and treatment of viral infection by Vaccinia Virus. Specific functions, and accordingly utilities, of VGAM246 correlate with, and may be deduced from, the identity of the host target genes which VGAM246 binds and inhibits, and the function of these host target genes, as elaborated hereinbelow.

Nucleotide sequences of the VGAM246 precursor RNA, herein designated VGAM PRECURSOR RNA, and of the ‘diced’ VGAM246 RNA, herein designated VGAM RNA, and a schematic representation of the secondary folding of VGAM246 folded precursor RNA, herein designated VGAM FOLDED PRECURSOR RNA, of VGAM246 are further described hereinbelow with reference to Table 1.

Nucleotide sequences of host target binding sites, such as BINDING SITE-I, BINDING SITE-II and BINDING SITE-III of FIG. 1, found on VGAM246 host target RNA, and schematic representation of the complementarity of each of these host target binding sites to VGAM246 RNA, herein designated VGAM RNA, are described hereinbelow with reference to Table 2.

As mentioned hereinabove with reference to FIG. 1, a function of VGAM246 gene, herein designated VGAM is inhibition of expression of VGAM246 target genes. It is appreciated that specific functions, and accordingly utilities, of VGAM246 correlate with, and may be deduced from, the identity of the target genes which VGAM246 binds and inhibits, and the function of these target genes, as elaborated hereinbelow.

Chemokine (C-C motif) Receptor-like 1 (CCRL1, Accession NM_{—}016557) is a VGAM246 host target gene. CCRL1 BINDING SITE is HOST TARGET binding site found in the 3′ untranslated region of mRNA encoded by CCRL1, corresponding to a HOST TARGET binding site such as BINDING SITE I, BINDING SITE II or BINDING SITE III. Table 2 illustrates the complementarity of the nucleotide sequences of CCRL1 BINDING SITE, designated SEQ ID:1697, to the nucleotide sequence of VGAM246 RNA, herein designated VGAM RNA, also designated SEQ ID:581.

A function of VGAM246 is therefore inhibition of Chemokine (C-C motif) Receptor-like 1 (CCRL1, Accession NM_{—}016557), a gene which is a G protein-coupled receptor that binds chemokines of the CC sub family, especially MCP-4, ELC (SCYA19) and TECK (SCYA25). Accordingly, utilities of VGAM246 include diagnosis, prevention and treatment of diseases and clinical conditions associated with CCRL1. The function of CCRL1 has been established by previous studies. Chemokine receptors are members of the 7-transmembrane-spanning, G protein-coupled receptor family that recognize small proteins responsible for the directed migration of specific cell types. Depending on the number of amino acids between the first 2 cysteines of their ligands (they may have more than 1 ligand), chemokine receptors are designated CCR (adjacent cysteines), CXCR (1 amino acid between the cysteines), or CX3CR (3 amino acids between the cysteines). The ‘R’ designation refers to proteins that not only bind, but also have a signaling function after binding. By searching an EST database for PPR1 homologs, Schweickart et al. (2000) obtained a cDNA encoding CCRL1, which they called CCR11. They initially reported that CCRL1 shares functional similarity to CCR2 (OMIM Ref. No. 601267) because it has a chemotactic response to MCP family chemokines (e.g., MCP2; 602283). However, in an erratum, Schweickart et al. (2000) corrected their functional data and stated that cells expressing CCRL1 do not have a chemotactic response to MCP family chemokines. They confirmed that CCRL1 binds ELC, SLC, and TECK, as reported by Gosling et al. (2000).

Full details of the abovementioned studies are described in the following publications, the disclosure of which are hereby incorporated by reference:
Schweickart, V. L.; Epp, A.; Raport, C. J.; Gray, P. W.: CCR11 is a functional receptor for the monocyte chemoattractant protein family of chemokines. J. Biol. Chem. 275: 9550-9556, 2000. Note: Erratum: J. Biol. Chem. 276: 856 only, 2001.; and
Gosling, J.; Dairaghi, D. J.; Wang, Y.; Hanley, M.; Talbot, D.; Miao, Z.; Schall, T. J.: Cutting edge: identification of a novel chemokine receptor that binds dendritic cell- and T cel.

Further studies establishing the function and utilities of CCRL1 are found in John Hopkins OMIM database record ID 606065, and in sited publications numbered 1599-1476 listed in the bibliography section hereinbelow, which are also hereby incorporated by reference. Platelet-activating Factor Acetylhydrolase, Isoform 1b, Alpha Subunit 45 kDa (PAFAH1B1, Accession NM_{—}000430) is another VGAM246 host target gene. PAFAH1B1 BINDING SITE is HOST TARGET binding site found in the 3′ untranslated region of mRNA encoded by PAFAH1B1, corresponding to a HOST TARGET binding site such as BINDING SITE I, BINDING SITE II or BINDING SITE III. Table 2 illustrates the complementarity of the nucleotide sequences of PAFAH1B1 BINDING SITE, designated SEQ ID:741, to the nucleotide sequence of VGAM246 RNA, herein designated VGAM RNA, also designated SEQ ID:581.

Another function of VGAM246 is therefore inhibition of Platelet-activating Factor Acetylhydrolase, Isoform 1b, Alpha Subunit 45 kDa (PAFAH1B1, Accession NM_{—}000430). Accordingly, utilities of VGAM246 include diagnosis, prevention and treatment of diseases and clinical conditions associated with PAFAH1B1. Proteasome (prosome, macropain) Subunit, Beta Type, 9 (large multifunctional protease 2) (PSMB9, Accession NM_{—}002800) is another VGAM246 host target gene. PSMB9 BINDING SITE is HOST TARGET binding site found in the 3′ untranslated region of mRNA encoded by PSMB9, corresponding to a HOST TARGET binding site such as BINDING SITE I, BINDING SITE II or BINDING SITE III. Table 2 illustrates the complementarity of the nucleotide sequences of PSMB9 BINDING SITE, designated SEQ ID:956, to the nucleotide sequence of VGAM246 RNA, herein designated VGAM RNA, also designated SEQ ID:581.

Another function of VGAM246 is therefore inhibition of Proteasome (prosome, macropain) Subunit, Beta Type, 9 (large multifunctional protease 2) (PSMB9, Accession NM_{—}002800), a gene which is one component of a multicatalytic proteinase complex. Accordingly, utilities of VGAM246 include diagnosis, prevention and treatment of diseases and clinical conditions associated with PSMB9. The function of PSMB9 has been established by previous studies. Driscoll et al. (1993) showed that the MHC-linked LMP2 and LMP7 subunits function to amplify specific endopeptidase activities of the proteasome. Gaczynska et al. (1993) presented experiments suggesting that gamma-interferon and expression of the LMP2 and LMP7 genes should favor the production by proteasomes of the types of peptides found on MHC class I molecules, which terminate almost exclusively with hydrophobic or basic residues. Animal model experiments lend further support to the function of PSMB9. Van Kaer et al. (1994) generated healthy mice with disrupted Lmp2 genes. Proteasomal peptidase activity against hydrophobic and basic substrates but not acidic substrates was lower in spleen and liver from mutant mice compared with wildtype mice. Differences in muscle and brain were not significant. Although flow cytometric analysis showed no difference in MHC class I expression, antigen-presenting cells from mutant mice were less able to stimulate a T-cell hybridoma specific for a nucleoprotein (NP) envelope antigen of an influenza A virus. Mutant mice also had less than half of the wildtype levels of CD8 (see OMIM Ref. No. 186910)-positive T lymphocytes and generated much lower levels of cytotoxic T-cell precursors specific for NP, though not for ovalbumin. Van Kaer et al. (1994) concluded that LMP2 selectively influences antigen processing of MHC class I-restricted antigens.

It is appreciated that the abovementioned animal model for PSMB9 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Van Kaer, L.; Ashton-Rickardt, P. G.; Eichelberger, M.; Gaczynska, M.; Nagashima, K.; Rock, K. L.; Goldberg, A. L.; Doherty, P. C.; Tonegawa, S.: Altered peptidase and viral-specific T cell response in LMP2 mutant mice. Immunity 1: 533-541, 1994.; and
Driscoll, J.; Brown, M. G.; Finley, D.; Monaco, J. J.: MHClinked LMP gene products specifically alter peptidase activities of the proteasome. Nature 365: 262-264, 1993.

Further studies establishing the function and utilities of PSMB9 are found in John Hopkins OMIM database record ID 177045, and in sited publications numbered 285-288, 94 and 2182-2186 listed in the bibliography section hereinbelow, which are also hereby incorporated by reference. Solute Carrier Family 4, Sodium Bicarbonate Cotransporter, Member 4 (SLC4A4, Accession NM_{—}003759) is another VGAM246 host target gene. SLC4A4 BINDING SITE is HOST TARGET binding site found in the 3′ untranslated region of mRNA encoded by SLC4A4, corresponding to a HOST TARGET binding site such as BINDING SITE I, BINDING SITE II or BINDING SITE III. Table 2 illustrates the complementarity of the nucleotide sequences of SLC4A4 BINDING SITE, designated SEQ ID:1055, to the nucleotide sequence of VGAM246 RNA, herein designated VGAM RNA, also designated SEQ ID:581.

Another function of VGAM246 is therefore inhibition of Solute Carrier Family 4, Sodium Bicarbonate Cotransporter, Member 4 (SLC4A4, Accession NM_{—}003759), a gene which is a sodium bicarbonate cotransporter. Accordingly, utilities of VGAM246 include diagnosis, prevention and treatment of diseases and clinical conditions associated with SLC4A4. The function of SLC4A4 and its association with various diseases and clinical conditions, has been established by previous studies, as described hereinabove with reference to VGAM49. Tumor Necrosis Factor, Alpha-induced Protein 1 (endothelial) (TNFAIP1, Accession NM_{—}021137) is another VGAM246 host target gene. TNFAIP1 BINDING SITE is HOST TARGET binding site found in the 3′ untranslated region of mRNA encoded by TNFAIP1, corresponding to a HOST TARGET binding site such as BINDING SITE I, BINDING SITE II or BINDING SITE III. Table 2 illustrates the complementarity of the nucleotide sequences of TNFAIP1 BINDING SITE, designated SEQ ID:1944, to the nucleotide sequence of VGAM246 RNA, herein designated VGAM RNA, also designated SEQ ID:581.

Another function of VGAM246 is therefore inhibition of Tumor Necrosis Factor, Alpha-induced Protein 1 (endothelial) (TNFAIP1, Accession NM_{—}021137). Accordingly, utilities of VGAM246 include diagnosis, prevention and treatment of diseases and clinical conditions associated with TNFAIP1. CAT56 (Accession NM_{—}025263) is another VGAM246 host target gene. CAT56 BINDING SITE is HOST TARGET binding site found in the 3′ untranslated region of mRNA encoded by CAT56, corresponding to a HOST TARGET binding site such as BINDING SITE I, BINDING SITE II or BINDING SITE III. Table 2 illustrates the complementarity of the nucleotide sequences of CAT56 BINDING SITE, designated SEQ ID:2149, to the nucleotide sequence of VGAM246 RNA, herein designated VGAM RNA, also designated SEQ ID:581.

Another function of VGAM246 is therefore inhibition of CAT56 (Accession NM_{—}025263). Accordingly, utilities of VGAM246 include diagnosis, prevention and treatment of diseases and clinical conditions associated with CAT56. FLJ10520 (Accession NM_{—}018124) is another VGAM246 host target gene. FLJ10520 BINDING SITE is HOST TARGET binding site found in the 3′ untranslated region of mRNA encoded by FLJ10520, corresponding to a HOST TARGET binding site such as BINDING SITE I, BINDING SITE II or BINDING SITE III. Table 2 illustrates the complementarity of the nucleotide sequences of FLJ10520 BINDING SITE, designated SEQ ID:1785, to the nucleotide sequence of VGAM246 RNA, herein designated VGAM RNA, also designated SEQ ID:581.

Another function of VGAM246 is therefore inhibition of FLJ10520 (Accession NM_{—}018124). Accordingly, utilities of VGAM246 include diagnosis, prevention and treatment of diseases and clinical conditions associated with FLJ10520. FLJ14001 (Accession NM_{—}024677) is another VGAM246 host target gene. FLJ14001 BINDING SITE is HOST TARGET binding site found in the 5′ untranslated region of mRNA encoded by FLJ14001, corresponding to a HOST TARGET binding site such as BINDING SITE I, BINDING SITE II or BINDING SITE III. Table 2 illustrates the complementarity of the nucleotide sequences of FLJ14001 BINDING SITE, designated SEQ ID:2081, to the nucleotide sequence of VGAM246 RNA, herein designated VGAM RNA, also designated SEQ ID:581.

Another function of VGAM246 is therefore inhibition of FLJ14001 (Accession NM_{—}024677). Accordingly, utilities of VGAM246 include diagnosis, prevention and treatment of diseases and clinical conditions associated with FLJ14001. FLJ14917 (Accession NM_{—}032861) is another VGAM246 host target gene. FLJ14917 BINDING SITE is HOST TARGET binding site found in the 3′ untranslated region of mRNA encoded by FLJ14917, corresponding to a HOST TARGET binding site such as BINDING SITE I, BINDING SITE II or BINDING SITE III. Table 2 illustrates the complementarity of the nucleotide sequences of FLJ14917 BINDING SITE, designated SEQ ID:2290, to the nucleotide sequence of VGAM246 RNA, herein designated VGAM RNA, also designated SEQ ID:581.

Another function of VGAM246 is therefore inhibition of FLJ14917 (Accession NM_{—}032861). Accordingly, utilities of VGAM246 include diagnosis, prevention and treatment of diseases and clinical conditions associated with FLJ14917. KIAA0618 (Accession NM_{—}014833) is another VGAM246 host target gene. KIAA0618 BINDING SITE is HOST TARGET binding site found in the 3′ untranslated region of mRNA encoded by KIAA0618, corresponding to a HOST TARGET binding site such as BINDING SITE I, BINDING SITE II or BINDING SITE III. Table 2 illustrates the complementarity of the nucleotide sequences of KIAA0618 BINDING SITE, designated SEQ ID:1564, to the nucleotide sequence of VGAM246 RNA, herein designated VGAM RNA, also designated SEQ ID:581.

Another function of VGAM246 is therefore inhibition of KIAA0618 (Accession NM_{—}014833). Accordingly, utilities of VGAM246 include diagnosis, prevention and treatment of diseases and clinical conditions associated with KIAA0618. KIAA1750 (Accession XM_{—}043067) is another VGAM246 host target gene. KIAA1750 BINDING SITE is HOST TARGET binding site found in the 3′ untranslated region of mRNA encoded by KIAA1750, corresponding to a HOST TARGET binding site such as BINDING SITE I, BINDING SITE II or BINDING SITE III. Table 2 illustrates the complementarity of the nucleotide sequences of KIAA1750 BINDING SITE, designated SEQ ID:2812, to the nucleotide sequence of VGAM246 RNA, herein designated VGAM RNA, also designated SEQ ID:581.

Another function of VGAM246 is therefore inhibition of KIAA1750 (Accession XM_{—}043067). Accordingly, utilities of VGAM246 include diagnosis, prevention and treatment of diseases and clinical conditions associated with KIAA1750. MAGEF1 (Accession XM_{—}011074) is another VGAM246 host target gene. MAGEF1 BINDING SITE is HOST TARGET binding site found in the 3′ untranslated region of mRNA encoded by MAGEF1, corresponding to a HOST TARGET binding site such as BINDING SITE I, BINDING SITE II or BINDING SITE III. Table 2 illustrates the complementarity of the nucleotide sequences of MAGEF1 BINDING SITE, designated SEQ ID:2555, to the nucleotide sequence of VGAM246 RNA, herein designated VGAM RNA, also designated SEQ ID:581.

Another function of VGAM246 is therefore inhibition of MAGEF1 (Accession XM_{—}011074). Accordingly, utilities of VGAM246 include diagnosis, prevention and treatment of diseases and clinical conditions associated with MAGEF1. LOC253017 (Accession XM_{—}171068) is another VGAM246 host target gene. LOC253017 BINDING SITE is HOST TARGET binding site found in the 5′ untranslated region of mRNA encoded by LOC253017, corresponding to a HOST TARGET binding site such as BINDING SITE I, BINDING SITE II or BINDING SITE III. Table 2 illustrates the complementarity of the nucleotide sequences of LOC253017 BINDING SITE, designated SEQ ID:3685, to the nucleotide sequence of VGAM246 RNA, herein designated VGAM RNA, also designated SEQ ID:581.

Another function of VGAM246 is therefore inhibition of LOC253017 (Accession XM_{—}171068). Accordingly, utilities of VGAM246 include diagnosis, prevention and treatment of diseases and clinical conditions associated with LOC253017. LOC51705 (Accession NM_{—}016242) is another VGAM246 host target gene. LOC51705 BINDING SITE is HOST TARGET binding site found in the 3′ untranslated region of mRNA encoded by LOC51705, corresponding to a HOST TARGET binding site such as BINDING SITE I, BINDING SITE II or BINDING SITE III. Table 2 illustrates the complementarity of the nucleotide sequences of LOC51705 BINDING SITE, designated SEQ ID: 1674, to the nucleotide sequence of VGAM246 RNA, herein designated VGAM RNA, also designated SEQ ID:581.

Another function of VGAM246 is therefore inhibition of LOC51705 (Accession NM_{—}016242). Accordingly, utilities of VGAM246 include diagnosis, prevention and treatment of diseases and clinical conditions associated with LOC51705. FIG. 1 further provides a conceptual description of a novel bioinformatically detected viral gene of the present invention, referred to here as Viral Genomic Address Messenger 247 (VGAM247) viral gene, which modulates expression of respective host target genes thereof, the function and utility of which host target genes is known in the art.

VGAM247 is a novel bioinformatically detected regulatory, non protein coding, viral micro RNA (miRNA) gene. The method by which VGAM247 was detected is described hereinabove with reference to FIGS. 1-8.

VGAM247 gene, herein designated VGAM GENE, is a viral gene contained in the genome of Vaccinia Virus. VGAM247 host target gene, herein designated VGAM HOST TARGET GENE, is a human gene contained in the human genome.

VGAM247 gene encodes a VGAM247 precursor RNA, herein designated VGAM PRECURSOR RNA. Similar to other miRNA genes, and unlike most ordinary genes, VGAM247 precursor RNA does not encode a protein. A nucleotide sequence identical or highly similar to the nucleotide sequence of VGAM247 precursor RNA is designated SEQ ID:233, and is provided hereinbelow with reference to the sequence listing part. Nucleotide sequence SEQ ID:233 is located at position 138880 relative to the genome of Vaccinia Virus.

VGAM247 precursor RNA folds onto itself, forming VGAM247 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM247 folded precursor RNA into VGAM247 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM247 RNA is designated SEQ ID:582, and is provided hereinbelow with reference to the sequence listing part.

VGAM247 host target gene, herein designated VGAM HOST TARGET GENE, encodes a corresponding messenger RNA, VGAM247 host target RNA, herein designated VGAM HOST TARGET RNA. VGAM247 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM247 RNA, herein designated VGAM RNA, binds complementarily to one or more host target binding sites located in untranslated regions of VGAM247 host target RNA, herein designated VGAM HOST TARGET RNA. This complementary binding is due to the fact that the nucleotide sequence of VGAM247 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM247 RNA, herein designated VGAM RNA, may have a different number of host target binding sites in untranslated regions of a VGAM247 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM247 RNA, herein designated VGAM RNA, to host target binding sites on VGAM247 host target RNA, herein designated VGAM HOST TARGET RNA, such as BINDING SITE I, BINDING SITE II and BINDING SITE III, inhibits translation of VGAM247 host target RNA into VGAM247 host target protein, herein designated VGAM HOST TARGET PROTEIN. VGAM host target protein is therefore outlined by a broken line.

It is appreciated that VGAM247 host target gene, herein designated VGAM HOST TARGET GENE, in fact represents a plurality of VGAM247 host target genes. The mRNA of each one of this plurality of VGAM247 host target genes comprises one or more host target binding sites, each having a nucleotide sequence which is at least partly complementary to VGAM247 RNA, herein designated VGAM RNA, and which when bound by VGAM247 RNA causes inhibition of translation of respective one or more VGAM247 host target proteins.

It is further appreciated by one skilled in the art that the mode of translational inhibition illustrated by FIG. 1 with specific reference to translational inhibition exerted by VGAM247 gene, herein designated VGAM GENE, on one or more VGAM247 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM247 is inhibition of expression of host target genes, as part of a novel viral mechanism of attacking a host. Accordingly, utilities of VGAM247 include diagnosis, prevention and treatment of viral infection by Vaccinia Virus. Specific functions, and accordingly utilities, of VGAM247 correlate with, and may be deduced from, the identity of the host target genes which VGAM247 binds and inhibits, and the function of these host target genes, as elaborated hereinbelow.

Nucleotide sequences of the VGAM247 precursor RNA, herein designated VGAM PRECURSOR RNA, and of the ‘diced’ VGAM247 RNA, herein designated VGAM RNA, and a schematic representation of the secondary folding of VGAM247 folded precursor RNA, herein designated VGAM FOLDED PRECURSOR RNA, of VGAM247 are further described hereinbelow with reference to Table 1.

Nucleotide sequences of host target binding sites, such as BINDING SITE-I, BINDING SITE-II and BINDING SITE-III of FIG. 1, found on VGAM247 host target RNA, and schematic representation of the complementarity of each of these host target binding sites to VGAM247 RNA, herein designated VGAM RNA, are described hereinbelow with reference to Table 2.

As mentioned hereinabove with reference to FIG. 1, a function of VGAM247 gene, herein designated VGAM is inhibition of expression of VGAM247 target genes. It is appreciated that specific functions, and accordingly utilities, of VGAM247 correlate with, and may be deduced from, the identity of the target genes which VGAM247 binds and inhibits, and the function of these target genes, as elaborated hereinbelow.

B-cell CLL/lymphoma 11B (zinc finger protein) (BCL11B, Accession NM_{—}022898) is a VGAM247 host target gene. BCL11B BINDING SITE is HOST TARGET binding site found in the 3′ untranslated region of mRNA encoded by BCL11B, corresponding to a HOST TARGET binding site such as BINDING SITE I, BINDING SITE II or BINDING SITE III. Table 2 illustrates the complementarity of the nucleotide sequences of BCL11B BINDING SITE, designated SEQ ID:2019, to the nucleotide sequence of VGAM247 RNA, herein designated VGAM RNA, also designated SEQ ID:582.

A function of VGAM247 is therefore inhibition of B-cell CLL/lymphoma 11B (zinc finger protein) (BCL11B, Accession NM_{—}022898). Accordingly, utilities of VGAM247 include diagnosis, prevention and treatment of diseases and clinical conditions associated with BCL11B. Fibroblast Growth Factor Receptor 4 (FGFR4, Accession NM_{—}022963) is another VGAM247 host target gene. FGFR4 BINDING SITE1 and FGFR4 BINDING SITE2 are HOST TARGET binding sites found in untranslated regions of mRNA encoded by FGFR4, corresponding to HOST TARGET binding sites such as BINDING SITE I, BINDING SITE II or BINDING SITE III. Table 2 illustrates the complementarity of the nucleotide sequences of FGFR4 BINDING SITE1 and FGFR4 BINDING SITE2, designated SEQ ID:2022 and SEQ ID:887 respectively, to the nucleotide sequence of VGAM247 RNA, herein designated VGAM RNA, also designated SEQ ID:582.

Another function of VGAM247 is therefore inhibition of Fibroblast Growth Factor Receptor 4 (FGFR4, Accession NM_{—}022963), a gene which receptor tyrosine kinase, preferentially binds acidic FGF. Accordingly, utilities of VGAM247 include diagnosis, prevention and treatment of diseases and clinical conditions associated with FGFR4. The function of FGFR4 has been established by previous studies. Partanen et al. (1991) reported the cDNA cloning and analysis of a novel member of the fibroblast growth factor receptor (FGFR) gene family expressed in K562 erythroleukemia cells. Its deduced amino acid sequence was 55% identical with the previously characterized FGFRs, FLG (FGFR1; 136350) and BEK (OMIM Ref. No. 176943), and had the structural characteristics of an FGFR family member including 3 immunoglobulin-like domains in its extracellular part. The expression pattern of FGFR4 was found to be distinct from that of FLG and BEK and also distinct from that of FGFR3 (OMIM Ref. No. 134934), which they (Keegan et al., 1991) had also cloned from K562 erythroleukemia cells. To elucidate further the physiologic relevance of protein-tyrosine kinases and to search for additional members of the gene family as possible factors in carcinogenesis, Holtrich et al. (1991) amplified mRNA from lung tissue by the polymerase chain reaction (PCR) using PTK-specific primers followed by sequencing of the clones. They identified a novel protein-tyrosine kinase, which they called TKF (tyrosine kinase related to fibroblast growth factor receptor). Among a wide variety of cells and tissues tested, including human lymphocytes and macrophages, TKF was found to be expressed only in lung and in some tumors of lung origin as well as in malignancies not derived from lung tissues. Sequence comparison has demonstrated that TKF is identical to FGFR4 (Scott, 1999). By analysis of somatic cell hybrids and by in situ hybridization, Armstrong et al. (1992) mapped the FGFR4 gene to 5q33-qter, an area involved in leukemias and lymphomas. In a radiation hybrid mapping of 18 genes on distal 5q, Warrington et al. (1992) found that the FGFR4 gene lies distal to DRD1 with high probability. Assuming that the mapping of DRD1 is correct, FGFR4 would be located in the segment 5q35.1-qter. Using an interspecific backcross mapping panel, Avraham et al. (1994) mapped the Fgfr4 gene to mouse chromosome 13 in a region of homology of synteny with distal human 5q.

Full details of the abovementioned studies are described in the following publications, the disclosure of which are hereby incorporated by reference:
Holtrich, U.; Brauninger, A.; Strebhardt, K.; Rubsamen-Waigmann, H.: Two additional protein-tyrosine kinases expressed in human lung: fourth member of the fibroblast growth factor receptor family and an intracellular protein-tyrosine kinase. Proc. Nat. Acad. Sci. 88: 10411-10415, 1991.; and
Avraham, K. B.; Givol, D.; Avivi, A.; Yayon, A.; Copeland, N. G.; Jenkins, N. A.: Mapping of murine fibroblast growth factor receptors refines regions of homology between mouse and huma.

Further studies establishing the function and utilities of FGFR4 are found in John Hopkins OMIM database record ID 134935, and in sited publications numbered 993, 994-995, 273 and 996-1000 listed in the bibliography section hereinbelow, which are also hereby incorporated by reference. SSH2 (Accession XM_{—}030846) is another VGAM247 host target gene. SSH2 BINDING SITE is HOST TARGET binding site found in the 3′ untranslated region of mRNA encoded by SSH2, corresponding to a HOST TARGET binding site such as BINDING SITE I, BINDING SITE II or BINDING SITE III. Table 2 illustrates the complementarity of the nucleotide sequences of SSH2 BINDING SITE, designated SEQ ID:2625, to the nucleotide sequence of VGAM247 RNA, herein designated VGAM RNA, also designated SEQ ID:582.

Another function of VGAM247 is therefore inhibition of SSH2 (Accession XM_{—}030846). Accordingly, utilities of VGAM247 include diagnosis, prevention and treatment of diseases and clinical conditions associated with SSH2. FIG. 1 further provides a conceptual description of a novel bioinformatically detected viral gene of the present invention, referred to here as Viral Genomic Address Messenger 248 (VGAM248) viral gene, which modulates expression of respective host target genes thereof, the function and utility of which host target genes is known in the art.

VGAM248 is a novel bioinformatically detected regulatory, non protein coding, viral micro RNA (miRNA) gene. The method by which VGAM248 was detected is described hereinabove with reference to FIGS. 1-8.

VGAM248 gene, herein designated VGAM GENE, is a viral gene contained in the genome of Vaccinia Virus. VGAM248 host target gene, herein designated VGAM HOST TARGET GENE, is a human gene contained in the human genome.

VGAM248 gene encodes a VGAM248 precursor RNA, herein designated VGAM PRECURSOR RNA. Similar to other miRNA genes, and unlike most ordinary genes, VGAM248 precursor RNA does not encode a protein. A nucleotide sequence identical or highly similar to the nucleotide sequence of VGAM248 precursor RNA is designated SEQ ID:234, and is provided hereinbelow with reference to the sequence listing part. Nucleotide sequence SEQ ID:234 is located at position 138729 relative to the genome of Vaccinia Virus.

VGAM248 precursor RNA folds onto itself, forming VGAM248 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM248 folded precursor RNA into VGAM248 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM248 RNA is designated SEQ ID:583, and is provided hereinbelow with reference to the sequence listing part.

VGAM248 host target gene, herein designated VGAM HOST TARGET GENE, encodes a corresponding messenger RNA, VGAM248 host target RNA, herein designated VGAM HOST TARGET RNA. VGAM248 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM248 RNA, herein designated VGAM RNA, binds complementarily to one or more host target binding sites located in untranslated regions of VGAM248 host target RNA, herein designated VGAM HOST TARGET RNA. This complementary binding is due to the fact that the nucleotide sequence of VGAM248 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM248 RNA, herein designated VGAM RNA, may have a different number of host target binding sites in untranslated regions of a VGAM248 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM248 RNA, herein designated VGAM RNA, to host target binding sites on VGAM248 host target RNA, herein designated VGAM HOST TARGET RNA, such as BINDING SITE I, BINDING SITE II and BINDING SITE III, inhibits translation of VGAM248 host target RNA into VGAM248 host target protein, herein designated VGAM HOST TARGET PROTEIN. VGAM host target protein is therefore outlined by a broken line.

It is appreciated that VGAM248 host target gene, herein designated VGAM HOST TARGET GENE, in fact represents a plurality of VGAM248 host target genes. The mRNA of each one of this plurality of VGAM248 host target genes comprises one or more host target binding sites, each having a nucleotide sequence which is at least partly complementary to VGAM248 RNA, herein designated VGAM RNA, and which when bound by VGAM248 RNA causes inhibition of translation of respective one or more VGAM248 host target proteins.

It is further appreciated by one skilled in the art that the mode of translational inhibition illustrated by FIG. 1 with specific reference to translational inhibition exerted by VGAM248 gene, herein designated VGAM GENE, on one or more VGAM248 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM248 is inhibition of expression of host target genes, as part of a novel viral mechanism of attacking a host. Accordingly, utilities of VGAM248 include diagnosis, prevention and treatment of viral infection by Vaccinia Virus. Specific functions, and accordingly utilities, of VGAM248 correlate with, and may be deduced from, the identity of the host target genes which VGAM248 binds and inhibits, and the function of these host target genes, as elaborated hereinbelow.

Nucleotide sequences of the VGAM248 precursor RNA, herein designated VGAM PRECURSOR RNA, and of the ‘diced’ VGAM248 RNA, herein designated VGAM RNA, and a schematic representation of the secondary folding of VGAM248 folded precursor RNA, herein designated VGAM FOLDED PRECURSOR RNA, of VGAM248 are further described hereinbelow with reference to Table 1.

Nucleotide sequences of host target binding sites, such as BINDING SITE-I, BINDING SITE-II and BINDING SITE-III of FIG. 1, found on VGAM248 host target RNA, and schematic representation of the complementarity of each of these host target binding sites to VGAM248 RNA, herein designated VGAM RNA, are described hereinbelow with reference to Table 2.

As mentioned hereinabove with reference to FIG. 1, a function of VGAM248 gene, herein designated VGAM is inhibition of expression of VGAM248 target genes. It is appreciated that specific functions, and accordingly utilities, of VGAM248 correlate with, and may be deduced from, the identity of the target genes which VGAM248 binds and inhibits, and the function of these target genes, as elaborated hereinbelow.

Retinoic Acid Receptor, Beta (RARB, Accession NM_{—}000965) is a VGAM248 host target gene. RARB BINDING SITE1 and RARB BINDING SITE2 are HOST TARGET binding sites found in untranslated regions of mRNA encoded by RARB, corresponding to HOST TARGET binding sites such as BINDING SITE I, BINDING SITE II or BINDING SITE III. Table 2 illustrates the complementarity of the nucleotide sequences of RARB BINDING SITE1 and RARB BINDING SITE2, designated SEQ ID:792 and SEQ ID: 1663 respectively, to the nucleotide sequence of VGAM248 RNA, herein designated VGAM RNA, also designated SEQ ID:583.

A function of VGAM248 is therefore inhibition of Retinoic Acid Receptor, Beta (RARB, Accession NM_{—}000965), a gene which is one member of the steroid/thyroid hormone receptor family of ligand-activated transcription factors. Accordingly, utilities of VGAM248 include diagnosis, prevention and treatment of diseases and clinical conditions associated with RARB. The function of RARB has been established by previous studies. The 3 retinoic acid receptors, alpha (RARA; 180240), beta (RARB), and gamma (RARG; 180190), are members of the nuclear receptor super family. Retinoic acid was the first morphogen described in vertebrates. The RARA and RARB genes are more homologous to those of the 2 closely related thyroid hormone receptors THRA and THRB, located on chromosomes 17 and 3, respectively, than to any other members of the nuclear receptor family. These observations suggest that the thyroid hormone and retinoic acid receptors evolved by gene, and possibly chromosome, duplications from a common ancestor which itself diverged rather early in evolution from the common ancestor of the steroid receptor group of the family. The RARB gene, formerly symbolized HAP, maps to 3p24 by somatic cell hybridization and in situ hybridization. Benbrook et al. (1988) showed a predominant distribution in epithelial tissues and therefore used the designation RAR(epsilon). By in situ hybridization, Mattei et al. (1988) assigned the RARB gene to 3p24. Using deletion mapping, de The et al. (1990) identified a 27-bp fragment located 59-bp upstream of the transcriptional start, which confers retinoic acid responsiveness on the herpesvirus thymidine kinase promoter. They found indications that both alpha and beta receptors act through the same DNA sequence. Mattei et al. (1991) assigned the corresponding gene to chromosome 14, band A, in the mouse, and to chromosome 15 in the rat. Nadeau et al. (1992) confirmed assignment of the mouse homolog to the centromeric portion of chromosome 14. From a comparison of a hepatitis-B virus (HBV) integration site present in a particular human hepatocellular carcinoma (HCC; 114550) with the corresponding unoccupied site in the nontumorous tissue of the same liver, Dejean et al. (1986) found that HBV integration placed the viral sequence next to a liver cell sequence that bears a striking resemblance to both an oncogene, ERBA (OMIM Ref. No. 190120), and the supposed DNA-binding domain of the human glucocorticoid receptor (OMIM Ref. No. 138040) and estrogen receptor (OMIM Ref. No. 133430) genes. Dejean et al. (1986) suggested that this gene, usually silent or transcribed at a very low level in normal hepatocytes, becomes inappropriately expressed as a consequence of HBV integration, thus contributing to the cell transformation. By means of a panel of rodent-human somatic cell hybrid DNAs, Dejean et al. (1986) localized the gene to chromosome 3. Further studies by de The et al. (1987) suggested that the HAP gene product may be a novel ligand-responsive regulatory protein whose inappropriate expression in liver is related to hepatocellular carcinogenesis. Brand et al. (1988) showed that the novel protein called HAP (for HBV-activated protein) is a retinoic acid receptor. They referred to this receptor as the beta type (RARB) and mapped it to 3p25-p21.

Full details of the abovementioned studies are described in the following publications, the disclosure of which are hereby incorporated by reference:
Benbrook, D.; Lernhardt, E.; Pfahl, M.: A new retinoic acid receptor identified from a hepatocellular carcinoma. (Letter) Nature 333: 669-672, 1988.; and
de The, H.; Marchio, A.; Tiollais, P.; Dejean, A.: A novel steroid thyroid hormone receptor-related gene inappropriately expressed in human hepatocellular carcinoma. Nature 330: 667-670.

Further studies establishing the function and utilities of RARB are found in John Hopkins OMIM database record ID 180220, and in sited publications numbered 1319-1322, 1331-133 and 2548-1336 listed in the bibliography section hereinbelow, which are also hereby incorporated by reference. Zinc Finger Protein 337 (ZNF337, Accession XM_{—}042807) is another VGAM248 host target gene. ZNF337 BINDING SITE is HOST TARGET binding site found in the 3′ untranslated region of mRNA encoded by ZNF337, corresponding to a HOST TARGET binding site such as BINDING SITE I, BINDING SITE II or BINDING SITE III. Table 2 illustrates the complementarity of the nucleotide sequences of ZNF337 BINDING SITE, designated SEQ ID:2806, to the nucleotide sequence of VGAM248 RNA, herein designated VGAM RNA, also designated SEQ ID:583.

Another function of VGAM248 is therefore inhibition of Zinc Finger Protein 337 (ZNF337, Accession XM_{—}042807). Accordingly, utilities of VGAM248 include diagnosis, prevention and treatment of diseases and clinical conditions associated with ZNF337. LOC139231 (Accession XM_{—}060020) is another VGAM248 host target gene. LOC139231 BINDING SITE is HOST TARGET binding site found in the 3′ untranslated region of mRNA encoded by LOC139231, corresponding to a HOST TARGET binding site such as BINDING SITE I, BINDING SITE II or BINDING SITE III. Table 2 illustrates the complementarity of the nucleotide sequences of LOC139231 BINDING SITE, designated SEQ ID:3021, to the nucleotide sequence of VGAM248 RNA, herein designated VGAM RNA, also designated SEQ ID:583.

Another function of VGAM248 is therefore inhibition of LOC139231 (Accession XM_{—}060020). Accordingly, utilities of VGAM248 include diagnosis, prevention and treatment of diseases and clinical conditions associated with LOC139231. LOC221399 (Accession XM_{—}168134) is another VGAM248 host target gene. LOC221399 BINDING SITE is HOST TARGET binding site found in the 5′ untranslated region of mRNA encoded by LOC221399, corresponding to a HOST TARGET binding site such as BINDING SITE I, BINDING SITE II or BINDING SITE III. Table 2 illustrates the complementarity of the nucleotide sequences of LOC221399 BINDING SITE, designated SEQ ID:3622, to the nucleotide sequence of VGAM248 RNA, herein designated VGAM RNA, also designated SEQ ID:583.

Another function of VGAM248 is therefore inhibition of LOC221399 (Accession XM_{—}168134). Accordingly, utilities of VGAM248 include diagnosis, prevention and treatment of diseases and clinical conditions associated with LOC221399. FIG. 1 further provides a conceptual description of a novel bioinformatically detected viral gene of the present invention, referred to here as Viral Genomic Address Messenger 249 (VGAM249) viral gene, which modulates expression of respective host target genes thereof, the function and utility of which host target genes is known in the art.

VGAM249 is a novel bioinformatically detected regulatory, non protein coding, viral micro RNA (miRNA) gene. The method by which VGAM249 was detected is described hereinabove with reference to FIGS. 1-8.

VGAM249 gene, herein designated VGAM GENE, is a viral gene contained in the genome of Vaccinia Virus. VGAM249 host target gene, herein designated VGAM HOST TARGET GENE, is a human gene contained in the human genome.

VGAM249 gene encodes a VGAM249 precursor RNA, herein designated VGAM PRECURSOR RNA. Similar to other miRNA genes, and unlike most ordinary genes, VGAM249 precursor RNA does not encode a protein. A nucleotide sequence identical or highly similar to the nucleotide sequence of VGAM249 precursor RNA is designated SEQ ID:235, and is provided hereinbelow with reference to the sequence listing part. Nucleotide sequence SEQ ID:235 is located at position 139033 relative to the genome of Vaccinia Virus.

VGAM249 precursor RNA folds onto itself, forming VGAM249 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM249 folded precursor RNA into VGAM249 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM249 RNA is designated SEQ ID:584, and is provided hereinbelow with reference to the sequence listing part.

VGAM249 host target gene, herein designated VGAM HOST TARGET GENE, encodes a corresponding messenger RNA, VGAM249 host target RNA, herein designated VGAM HOST TARGET RNA. VGAM249 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM249 RNA, herein designated VGAM RNA, binds complementarily to one or more host target binding sites located in untranslated regions of VGAM249 host target RNA, herein designated VGAM HOST TARGET RNA. This complementary binding is due to the fact that the nucleotide sequence of VGAM249 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM249 RNA, herein designated VGAM RNA, may have a different number of host target binding sites in untranslated regions of a VGAM249 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM249 RNA, herein designated VGAM RNA, to host target binding sites on VGAM249 host target RNA, herein designated VGAM HOST TARGET RNA, such as BINDING SITE I, BINDING SITE II and BINDING SITE III, inhibits translation of VGAM249 host target RNA into VGAM249 host target protein, herein designated VGAM HOST TARGET PROTEIN. VGAM host target protein is therefore outlined by a broken line.

It is appreciated that VGAM249 host target gene, herein designated VGAM HOST TARGET GENE, in fact represents a plurality of VGAM249 host target genes. The mRNA of each one of this plurality of VGAM249 host target genes comprises one or more host target binding sites, each having a nucleotide sequence which is at least partly complementary to VGAM249 RNA, herein designated VGAM RNA, and which when bound by VGAM249 RNA causes inhibition of translation of respective one or more VGAM249 host target proteins.

It is further appreciated by one skilled in the art that the mode of translational inhibition illustrated by FIG. 1 with specific reference to translational inhibition exerted by VGAM249 gene, herein designated VGAM GENE, on one or more VGAM249 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM249 is inhibition of expression of host target genes, as part of a novel viral mechanism of attacking a host. Accordingly, utilities of VGAM249 include diagnosis, prevention and treatment of viral infection by Vaccinia Virus. Specific functions, and accordingly utilities, of VGAM249 correlate with, and may be deduced from, the identity of the host target genes which VGAM249 binds and inhibits, and the function of these host target genes, as elaborated hereinbelow.

Nucleotide sequences of the VGAM249 precursor RNA, herein designated VGAM PRECURSOR RNA, and of the ‘diced’ VGAM249 RNA, herein designated VGAM RNA, and a schematic representation of the secondary folding of VGAM249 folded precursor RNA, herein designated VGAM FOLDED PRECURSOR RNA, of VGAM249 are further described hereinbelow with reference to Table 1.

Nucleotide sequences of host target binding sites, such as BINDING SITE-I, BINDING SITE-II and BINDING SITE-III of FIG. 1, found on VGAM249 host target RNA, and schematic representation of the complementarity of each of these host target binding sites to VGAM249 RNA, herein designated VGAM RNA, are described hereinbelow with reference to Table 2.

As mentioned hereinabove with reference to FIG. 1, a function of VGAM249 gene, herein designated VGAM is inhibition of expression of VGAM249 target genes. It is appreciated that specific functions, and accordingly utilities, of VGAM249 correlate with, and may be deduced from, the identity of the target genes which VGAM249 binds and inhibits, and the function of these target genes, as elaborated hereinbelow.

DKFZp762E1511 (Accession XM_{—}003460) is a VGAM249 host target gene. DKFZp762E1511 BINDING SITE is HOST TARGET binding site found in the 3′ untranslated region of mRNA encoded by DKFZp762E1511, corresponding to a HOST TARGET binding site such as BINDING SITE I, BINDING SITE II or BINDING SITE III. Table 2 illustrates the complementarity of the nucleotide sequences of DKFZp762E1511 BINDING SITE, designated SEQ ID:2530, to the nucleotide sequence of VGAM249 RNA, herein designated VGAM RNA, also designated SEQ ID:584.

A function of VGAM249 is therefore inhibition of DKFZp762E1511 (Accession XM_{—}003460). Accordingly, utilities of VGAM249 include diagnosis, prevention and treatment of diseases and clinical conditions associated with DKFZp762E1511. LOC123591 (Accession XM_{—}063741) is another VGAM249 host target gene. LOC123591 BINDING SITE is HOST TARGET binding site found in the 5′ untranslated region of mRNA encoded by LOC123591, corresponding to a HOST TARGET binding site such as BINDING SITE I, BINDING SITE II or BINDING SITE III. Table 2 illustrates the complementarity of the nucleotide sequences of LOC123591 BINDING SITE, designated SEQ ID:3032, to the nucleotide sequence of VGAM249 RNA, herein designated VGAM RNA, also designated SEQ ID:584.

Another function of VGAM249 is therefore inhibition of LOC123591 (Accession XM_{—}063741). Accordingly, utilities of VGAM249 include diagnosis, prevention and treatment of diseases and clinical conditions associated with LOC123591. FIG. 1 further provides a conceptual description of a novel bioinformatically detected viral gene of the present invention, referred to here as Viral Genomic Address Messenger 250 (VGAM250) viral gene, which modulates expression of respective host target genes thereof, the function and utility of which host target genes is known in the art.

VGAM250 is a novel bioinformatically detected regulatory, non protein coding, viral micro RNA (miRNA) gene. The method by which VGAM250 was detected is described hereinabove with reference to FIGS. 1-8.

VGAM250 gene, herein designated VGAM GENE, is a viral gene contained in the genome of Vaccinia Virus. VGAM250 host target gene, herein designated VGAM HOST TARGET GENE, is a human gene contained in the human genome.

VGAM250 gene encodes a VGAM250 precursor RNA, herein designated VGAM PRECURSOR RNA. Similar to other miRNA genes, and unlike most ordinary genes, VGAM250 precursor RNA does not encode a protein. A nucleotide sequence identical or highly similar to the nucleotide sequence of VGAM250 precursor RNA is designated SEQ ID:236, and is provided hereinbelow with reference to the sequence listing part. Nucleotide sequence SEQ ID:236 is located at position 139228 relative to the genome of Vaccinia Virus.

VGAM250 precursor RNA folds onto itself, forming VGAM250 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM250 folded precursor RNA into VGAM250 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM250 RNA is designated SEQ ID:585, and is provided hereinbelow with reference to the sequence listing part.

VGAM250 host target gene, herein designated VGAM HOST TARGET GENE, encodes a corresponding messenger RNA, VGAM250 host target RNA, herein designated VGAM HOST TARGET RNA. VGAM250 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM250 RNA, herein designated VGAM RNA, binds complementarily to one or more host target binding sites located in untranslated regions of VGAM250 host target RNA, herein designated VGAM HOST TARGET RNA. This complementary binding is due to the fact that the nucleotide sequence of VGAM250 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM250 RNA, herein designated VGAM RNA, may have a different number of host target binding sites in untranslated regions of a VGAM250 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM250 RNA, herein designated VGAM RNA, to host target binding sites on VGAM250 host target RNA, herein designated VGAM HOST TARGET RNA, such as BINDING SITE I, BINDING SITE II and BINDING SITE III, inhibits translation of VGAM250 host target RNA into VGAM250 host target protein, herein designated VGAM HOST TARGET PROTEIN. VGAM host target protein is therefore outlined by a broken line.

It is appreciated that VGAM250 host target gene, herein designated VGAM HOST TARGET GENE, in fact represents a plurality of VGAM250 host target genes. The mRNA of each one of this plurality of VGAM250 host target genes comprises one or more host target binding sites, each having a nucleotide sequence which is at least partly complementary to VGAM250 RNA, herein designated VGAM RNA, and which when bound by VGAM250 RNA causes inhibition of translation of respective one or more VGAM250 host target proteins.

It is further appreciated by one skilled in the art that the mode of translational inhibition illustrated by FIG. 1 with specific reference to translational inhibition exerted by VGAM250 gene, herein designated VGAM GENE, on one or more VGAM250 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM250 is inhibition of expression of host target genes, as part of a novel viral mechanism of attacking a host. Accordingly, utilities of VGAM250 include diagnosis, prevention and treatment of viral infection by Vaccinia Virus. Specific functions, and accordingly utilities, of VGAM250 correlate with, and may be deduced from, the identity of the host target genes which VGAM250 binds and inhibits, and the function of these host target genes, as elaborated hereinbelow.

Nucleotide sequences of the VGAM250 precursor RNA, herein designated VGAM PRECURSOR RNA, and of the ‘diced’ VGAM250 RNA, herein designated VGAM RNA, and a schematic representation of the secondary folding of VGAM250 folded precursor RNA, herein designated VGAM FOLDED PRECURSOR RNA, of VGAM250 are further described hereinbelow with reference to Table 1.

Nucleotide sequences of host target binding sites, such as BINDING SITE-I, BINDING SITE-II and BINDING SITE-III of FIG. 1, found on VGAM250 host target RNA, and schematic representation of the complementarity of each of these host target binding sites to VGAM250 RNA, herein designated VGAM RNA, are described hereinbelow with reference to Table 2.

As mentioned hereinabove with reference to FIG. 1, a function of VGAM250 gene, herein designated VGAM is inhibition of expression of VGAM250 target genes. It is appreciated that specific functions, and accordingly utilities, of VGAM250 correlate with, and may be deduced from, the identity of the target genes which VGAM250 binds and inhibits, and the function of these target genes, as elaborated hereinbelow.

Ephrin-B2 (EFNB2, Accession NM_{—}004093) is a VGAM250 host target gene. EFNB2 BINDING SITE is HOST TARGET binding site found in the 3′ untranslated region of mRNA encoded by EFNB2, corresponding to a HOST TARGET binding site such as BINDING SITE I, BINDING SITE II or BINDING SITE III. Table 2 illustrates the complementarity of the nucleotide sequences of EFNB2 BINDING SITE, designated SEQ ID: 1085, to the nucleotide sequence of VGAM250 RNA, herein designated VGAM RNA, also designated SEQ ID:585.

A function of VGAM250 is therefore inhibition of Ephrin-B2 (EFNB2, Accession NM_{—}004093). Accordingly, utilities of VGAM250 include diagnosis, prevention and treatment of diseases and clinical conditions associated with EFNB2. Fatty-acid-Coenzyme A Ligase, Long-chain 4 (FACL4, Accession NM_{—}022977) is another VGAM250 host target gene. FACL4 BINDING SITE is HOST TARGET binding site found in the 5′ untranslated region of mRNA encoded by FACL4, corresponding to a HOST TARGET binding site such as BINDING SITE I, BINDING SITE II or BINDING SITE III. Table 2 illustrates the complementarity of the nucleotide sequences of FACL4 BINDING SITE, designated SEQ ID:2025, to the nucleotide sequence of VGAM250 RNA, herein designated VGAM RNA, also designated SEQ ID:585.

Another function of VGAM250 is therefore inhibition of Fatty-acid-Coenzyme A Ligase, Long-chain 4 (FACL4, Accession NM_{—}022977). Accordingly, utilities of VGAM250 include diagnosis, prevention and treatment of diseases and clinical conditions associated with FACL4. GATA Binding Protein 2 (GATA2, Accession NM_{—}002050) is another VGAM250 host target gene. GATA2 BINDING SITE is HOST TARGET binding site found in the 3′ untranslated region of mRNA encoded by GATA2, corresponding to a HOST TARGET binding site such as BINDING SITE I, BINDING SITE II or BINDING SITE III. Table 2 illustrates the complementarity of the nucleotide sequences of GATA2 BINDING SITE, designated SEQ ID:892, to the nucleotide sequence of VGAM250 RNA, herein designated VGAM RNA, also designated SEQ ID:585.

Another function of VGAM250 is therefore inhibition of GATA Binding Protein 2 (GATA2, Accession NM_{—}002050). Accordingly, utilities of VGAM250 include diagnosis, prevention and treatment of diseases and clinical conditions associated with GATA2. Insulin Receptor Substrate 2 (IRS2, Accession XM_{—}007095) is another VGAM250 host target gene. IRS2 BINDING SITE is HOST TARGET binding site found in the 3′ untranslated region of mRNA encoded by IRS2, corresponding to a HOST TARGET binding site such as BINDING SITE I, BINDING SITE II or BINDING SITE III. Table 2 illustrates the complementarity of the nucleotide sequences of IRS2 BINDING SITE, designated SEQ ID:2540, to the nucleotide sequence of VGAM250 RNA, herein designated VGAM RNA, also designated SEQ ID:585.

Another function of VGAM250 is therefore inhibition of Insulin Receptor Substrate 2 (IRS2, Accession XM_{—}007095), a gene which may mediate the control of various cellular processes by insulin. Accordingly, utilities of VGAM250 include diagnosis, prevention and treatment of diseases and clinical conditions associated with IRS2. The function of IRS2 has been established by previous studies. The protein IRS1 (OMIM Ref. No. 147545) acts as an interface between signaling proteins with Src homology-2 domains (SH2 proteins) and the receptors for insulin (INS; 176730), IGF2 (OMIM Ref. No. 147470), growth hormone (GH1; 139250), several interleukins (IL4, 147780; IL9, 146931; IL13, 147683), and other cytokines. It regulates gene expression and stimulates mitogenesis and appears to mediate insulin/IGF1-stimulated glucose transport. Thus, the finding that survival of the homozygous Irs1 knockout mouse with only mild resistance to hypertension was surprising. This dilemma was provisionally resolved by the discovery by Sun et al. (1995) of a second IRS signaling protein in mouse. They purified and cloned a likely candidate from mouse myeloid progenitor cells and, because of its resemblance to IRS1, they designated it IRS2. Alignment of the sequences of IRS2 and IRS1 demonstrated a highly conserved N terminus containing a pleckstrin-homology domain and a phosphotyrosine-binding (PTB) domain, and a poorly conserved C terminus containing several tyrosine phosphorylation motifs. IRS2 is expressed in many cells, including tissues from the homozygous IRS1 knockout mouse. Sun et al. (1995) suggested that IRS2 may be essential for signaling by several receptor systems. Mammarella et al. (2000) genotyped 193 Italian patients with type II diabetes (OMIM Ref. No. 125853) and 206 control subjects for the IRS2 G1057D polymorphism (600797.0001). They found evidence for a strong association between type II diabetes and the polymorphism, which appears to be protective against type II diabetes in a codominant fashion Animal model experiments lend further support to the function of IRS2. To be et al. (2001) observed that Irs2-deficient mice (Kubota et al. (2000)) showed increased adiposity with increased serum leptin level, suggesting leptin resistance before the mice developed diabetes. Using oligonucleotide microarray and Northern blot analyses to analyze gene expression, To be et al. (2001) detected increased expression of SREBP1, a downstream target of insulin, in Irs2-deficient mouse liver. Using high dose leptin administration, They provided evidence that leptin resistance in Irs2-deficient mice is causally related to SREBP1 gene induction. The authors concluded that Irs2 gene disruption results in leptin resistance, causing SREBP1 gene induction, obesity, fatty liver, and diabetes

It is appreciated that the abovementioned animal model for IRS2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Sun, X. J.; Wang, L.-M.; Zhang, Y.; Yenush, L.; Myers, M. G., Jr.; Glasheen, E.; Lane, W. S.; Pierce, J. H.; White, M. F.: Role of IRS-2 in insulin and cytokine signalling. Nature 377: 173-177, 1995.; and
To be, K.; Suzuki, R.; Aoyama, M.; Yamauchi, T.; Kamon, J.; Kubota, N.; Terauchi, Y.; Matsui, J.; Akanuma, Y.; Kimura, S.; Tanaka, J.; Abe, M.; Ohsumi, J.; Nagai, R.; Kadowaki, T.: Incre.

Further studies establishing the function and utilities of IRS2 are found in John Hopkins OMIM database record ID 600797, and in sited publications numbered 17, 351-353, 2864, 249 and 3050-356 listed in the bibliography section hereinbelow, which are also hereby incorporated by reference. Phenylalanine Hydroxylase (PAH, Accession NM_{—}000277) is another VGAM250 host target gene. PAH BINDING SITE is HOST TARGET binding site found in the 5′ untranslated region of mRNA encoded by PAH, corresponding to a HOST TARGET binding site such as BINDING SITE I, BINDING SITE II or BINDING SITE III. Table 2 illustrates the complementarity of the nucleotide sequences of PAH BINDING SITE, designated SEQ ID:725, to the nucleotide sequence of VGAM250 RNA, herein designated VGAM RNA, also designated SEQ ID:585.

Another function of VGAM250 is therefore inhibition of Phenylalanine Hydroxylase (PAH, Accession NM_{—}000277). Accordingly, utilities of VGAM250 include diagnosis, prevention and treatment of diseases and clinical conditions associated with PAH. Podocalyxin-like (PODXL, Accession NM_{—}005397) is another VGAM250 host target gene. PODXL BINDING SITE is HOST TARGET binding site found in the 3′ untranslated region of mRNA encoded by PODXL, corresponding to a HOST TARGET binding site such as BINDING SITE I, BINDING SITE II or BINDING SITE III. Table 2 illustrates the complementarity of the nucleotide sequences of PODXL BINDING SITE, designated SEQ ID:1207, to the nucleotide sequence of VGAM250 RNA, herein designated VGAM RNA, also designated SEQ ID:585.

Another function of VGAM250 is therefore inhibition of Podocalyxin-like (PODXL, Accession NM_{—}005397), a gene which is an antiadhesin. Accordingly, utilities of VGAM250 include diagnosis, prevention and treatment of diseases and clinical conditions associated with PODXL. The function of PODXL and its association with various diseases and clinical conditions, has been established by previous studies, as described hereinabove with reference to VGAM165. Ribosomal Protein L15 (RPL15, Accession NM_{—}002948) is another VGAM250 host target gene. RPL15 BINDING SITE is HOST TARGET binding site found in the 3′ untranslated region of mRNA encoded by RPL15, corresponding to a HOST TARGET binding site such as BINDING SITE I, BINDING SITE II or BINDING SITE III. Table 2 illustrates the complementarity of the nucleotide sequences of RPL15 BINDING SITE, designated SEQ ID:973, to the nucleotide sequence of VGAM250 RNA, herein designated VGAM RNA, also designated SEQ ID:585.

Another function of VGAM250 is therefore inhibition of Ribosomal Protein L15 (RPL15, Accession NM_{—}002948). Accordingly, utilities of VGAM250 include diagnosis, prevention and treatment of diseases and clinical conditions associated with RPL15. Serine (or cysteine) Proteinase Inhibitor, Clade E (nexin, plasminogen activator inhibitor type 1), Member 1 (SERPINE1, Accession NM_{—}000602) is another VGAM250 host target gene. SERPINE1 BINDING SITE is HOST TARGET binding site found in the 3′ untranslated region of mRNA encoded by SERPINE1, corresponding to a HOST TARGET binding site such as BINDING SITE I, BINDING SITE II or BINDING SITE III. Table 2 illustrates the complementarity of the nucleotide sequences of SERPINE1 BINDING SITE, designated SEQ ID:758, to the nucleotide sequence of VGAM250 RNA, herein designated VGAM RNA, also designated SEQ ID:585.

Another function of VGAM250 is therefore inhibition of Serine (or cysteine) Proteinase Inhibitor, Clade E (nexin, plasminogen activator inhibitor type 1), Member 1 (SERPINE1, Accession NM_{—}000602). Accordingly, utilities of VGAM250 include diagnosis, prevention and treatment of diseases and clinical conditions associated with SERPINE1. ABIN-2 (Accession NM_{—}024309) is another VGAM250 host target gene. ABIN-2 BINDING SITE is HOST TARGET binding site found in the 3′ untranslated region of mRNA encoded by ABIN-2, corresponding to a HOST TARGET binding site such as BINDING SITE I, BINDING SITE II or BINDING SITE III. Table 2 illustrates the complementarity of the nucleotide sequences of ABIN-2 BINDING SITE, designated SEQ ID:2057, to the nucleotide sequence of VGAM250 RNA, herein designated VGAM RNA, also designated SEQ ID:585.

Another function of VGAM250 is therefore inhibition of ABIN-2 (Accession NM_{—}024309). Accordingly, utilities of VGAM250 include diagnosis, prevention and treatment of diseases and clinical conditions associated with ABIN-2. C20orf48 (Accession NM_{—}024960) is another VGAM250 host target gene. C20orf48 BINDING SITE is HOST TARGET binding site found in the 5′ untranslated region of mRNA encoded by C20orf48, corresponding to a HOST TARGET binding site such as BINDING SITE I, BINDING SITE II or BINDING SITE III. Table 2 illustrates the complementarity of the nucleotide sequences of C20orf48 BINDING SITE, designated SEQ ID:2118, to the nucleotide sequence of VGAM250 RNA, herein designated VGAM RNA, also designated SEQ ID:585.

Another function of VGAM250 is therefore inhibition of C20orf48 (Accession NM_{—}024960). Accordingly, utilities of VGAM250 include diagnosis, prevention and treatment of diseases and clinical conditions associated with C20orf48.

Chromosome 9 Open Reading Frame 7 (C9orf7, Accession NM_{—}017586) is another VGAM250 host target gene. C9orf7 BINDING SITE is HOST TARGET binding site found in the 3′ untranslated region of mRNA encoded by C9orf7, corresponding to a HOST TARGET binding site such as BINDING SITE I, BINDING SITE II or BINDING SITE III. Table 2 illustrates the complementarity of the nucleotide sequences of C9orf7 BINDING SITE, designated SEQ ID:1724, to the nucleotide sequence of VGAM250 RNA, herein designated VGAM RNA, also designated SEQ ID:585.

Another function of VGAM250 is therefore inhibition of Chromosome 9 Open Reading Frame 7 (C9orf7, Accession NM_{—}017586). Accordingly, utilities of VGAM250 include diagnosis, prevention and treatment of diseases and clinical conditions associated with C9orf7. Dual Specificity Phosphatase 10 (DUSP10, Accession NM_{—}007207) is another VGAM250 host target gene. DUSP10 BINDING SITE1 and DUSP10 BINDING SITE2 are HOST TARGET binding sites found in untranslated regions of mRNA encoded by DUSP10, corresponding to HOST TARGET binding sites such as BINDING SITE I, BINDING SITE II or BINDING SITE III. Table 2 illustrates the complementarity of the nucleotide sequences of DUSP10 BINDING SITE1 and DUSP10 BINDING SITE2, designated SEQ ID:1364 and SEQ ID:2501 respectively, to the nucleotide sequence of VGAM250 RNA, herein designated VGAM RNA, also designated SEQ ID:585.

Another function of VGAM250 is therefore inhibition of Dual Specificity Phosphatase 10 (DUSP10, Accession NM_{—}007207). Accordingly, utilities of VGAM250 include diagnosis, prevention and treatment of diseases and clinical conditions associated with DUSP10. FLJ12892 (Accession XM_{—}042173) is another VGAM250 host target gene. FLJ12892 BINDING SITE is HOST TARGET binding site found in the 3′ untranslated region of mRNA encoded by FLJ12892, corresponding to a HOST TARGET binding site such as BINDING SITE I, BINDING SITE II or BINDING SITE III. Table 2 illustrates the complementarity of the nucleotide sequences of FLJ12892 BINDING SITE, designated SEQ ID:2802, to the nucleotide sequence of VGAM250 RNA, herein designated VGAM RNA, also designated SEQ ID:585.

Another function of VGAM250 is therefore inhibition of FLJ12892 (Accession XM_{—}042173). Accordingly, utilities of VGAM250 include diagnosis, prevention and treatment of diseases and clinical conditions associated with FLJ12892. FLJ22059 (Accession NM_{—}022752) is another VGAM250 host target gene. FLJ22059 BINDING SITE is HOST TARGET binding site found in the 5′ untranslated region of mRNA encoded by FLJ22059, corresponding to a HOST TARGET binding site such as BINDING SITE I, BINDING SITE II or BINDING SITE III. Table 2 illustrates the complementarity of the nucleotide sequences of FLJ22059 BINDING SITE, designated SEQ ID:2003, to the nucleotide sequence of VGAM250 RNA, herein designated VGAM RNA, also designated SEQ ID:585.

Another function of VGAM250 is therefore inhibition of FLJ22059 (Accession NM_{—}022752). Accordingly, utilities of VGAM250 include diagnosis, prevention and treatment of diseases and clinical conditions associated with FLJ22059. HEMK (Accession NM_{—}016173) is another VGAM250 host target gene. HEMK BINDING SITE is HOST TARGET binding site found in the 3′ untranslated region of mRNA encoded by HEMK, corresponding to a HOST TARGET binding site such as BINDING SITE I, BINDING SITE II or BINDING SITE III. Table 2 illustrates the complementarity of the nucleotide sequences of HEMK BINDING SITE, designated SEQ ID:1669, to the nucleotide sequence of VGAM250 RNA, herein designated VGAM RNA, also designated SEQ ID:585.

Another function of VGAM250 is therefore inhibition of HEMK (Accession NM_{—}016173). Accordingly, utilities of VGAM250 include diagnosis, prevention and treatment of diseases and clinical conditions associated with HEMK. KIAA0227 (Accession XM_{—}027236) is another VGAM250 host target gene. KIAA0227 BINDING SITE is HOST TARGET binding site found in the 3′ untranslated region of mRNA encoded by KIAA0227, corresponding to a HOST TARGET binding site such as BINDING SITE I, BINDING SITE II or BINDING SITE III. Table 2 illustrates the complementarity of the nucleotide sequences of KIAA0227 BINDING SITE, designated SEQ ID:2578, to the nucleotide sequence of VGAM250 RNA, herein designated VGAM RNA, also designated SEQ ID:585.

Another function of VGAM250 is therefore inhibition of KIAA0227 (Accession XM_{—}027236). Accordingly, utilities of VGAM250 include diagnosis, prevention and treatment of diseases and clinical conditions associated with KIAA0227. KIAA0527 (Accession XM_{—}171054) is another VGAM250 host target gene. KIAA0527 BINDING SITE is HOST TARGET binding site found in the 3′ untranslated region of mRNA encoded by KIAA0527, corresponding to a HOST TARGET binding site such as BINDING SITE I, BINDING SITE II or BINDING SITE III. Table 2 illustrates the complementarity of the nucleotide sequences of KIAA0527 BINDING SITE, designated SEQ ID:3684, to the nucleotide sequence of VGAM250 RNA, herein designated VGAM RNA, also designated SEQ ID:585.

Another function of VGAM250 is therefore inhibition of KIAA0527 (Accession XM_{—}171054). Accordingly, utilities of VGAM250 include diagnosis, prevention and treatment of diseases and clinical conditions associated with KIAA0527. KIAA1244 (Accession XM_{—}050424) is another VGAM250 host target gene. KIAA1244 BINDING SITE is HOST TARGET binding site found in the 3′ untranslated region of mRNA encoded by KIAA1244, corresponding to a HOST TARGET binding site such as BINDING SITE I, BINDING SITE II or BINDING SITE III. Table 2 illustrates the complementarity of the nucleotide sequences of KIAA1244 BINDING SITE, designated SEQ ID:2930, to the nucleotide sequence of VGAM250 RNA, herein designated VGAM RNA, also designated SEQ ID:585.

Another function of VGAM250 is therefore inhibition of KIAA1244 (Accession XM_{—}050424). Accordingly, utilities of VGAM250 include diagnosis, prevention and treatment of diseases and clinical conditions associated with KIAA1244. KIAA1649 (Accession XM_{—}040095) is another VGAM250 host target gene. KIAA1649 BINDING SITE is HOST TARGET binding site found in the 3′ untranslated region of mRNA encoded by KIAA1649, corresponding to a HOST TARGET binding site such as BINDING SITE I, BINDING SITE II or BINDING SITE III. Table 2 illustrates the complementarity of the nucleotide sequences of KIAA1649 BINDING SITE, designated SEQ ID:2771, to the nucleotide sequence of VGAM250 RNA, herein designated VGAM RNA, also designated SEQ ID:585.

Another function of VGAM250 is therefore inhibition of KIAA1649 (Accession XM_{—}040095). Accordingly, utilities of VGAM250 include diagnosis, prevention and treatment of diseases and clinical conditions associated with KIAA1649. KIAA1981 (Accession XM_{—}114000) is another VGAM250 host target gene. KIAA1981 BINDING SITE is HOST TARGET binding site found in the 3′ untranslated region of mRNA encoded by KIAA1981, corresponding to a HOST TARGET binding site such as BINDING SITE I, BINDING SITE II or BINDING SITE III. Table 2 illustrates the complementarity of the nucleotide sequences of KIAA1981 BINDING SITE, designated SEQ ID:3431, to the nucleotide sequence of VGAM250 RNA, herein designated VGAM RNA, also designated SEQ ID:585.

Another function of VGAM250 is therefore inhibition of KIAA1981 (Accession XM_{—}114000). Accordingly, utilities of VGAM250 include diagnosis, prevention and treatment of diseases and clinical conditions associated with KIAA1981. MGC2306 (Accession NM_{—}032638) is another VGAM250 host target gene. MGC2306 BINDING SITE is HOST TARGET binding site found in the 3′ untranslated region of mRNA encoded by MGC2306, corresponding to a HOST TARGET binding site such as BINDING SITE I, BINDING SITE II or BINDING SITE III. Table 2 illustrates the complementarity of the nucleotide sequences of MGC2306 BINDING SITE, designated SEQ ID:2266, to the nucleotide sequence of VGAM250 RNA, herein designated VGAM RNA, also designated SEQ ID:585.

Another function of VGAM250 is therefore inhibition of MGC2306 (Accession NM_{—}032638). Accordingly, utilities of VGAM250 include diagnosis, prevention and treatment of diseases and clinical conditions associated with MGC2306. Ribosomal Protein L39-like (RPL39L, Accession NM_{—}052969) is another VGAM250 host target gene. RPL39L BINDING SITE is HOST TARGET binding site found in the 5′ untranslated region of mRNA encoded by RPL39L, corresponding to a HOST TARGET binding site such as BINDING SITE I, BINDING SITE II or BINDING SITE III. Table 2 illustrates the complementarity of the nucleotide sequences of RPL39L BINDING SITE, designated SEQ ID:2352, to the nucleotide sequence of VGAM250 RNA, herein designated VGAM RNA, also designated SEQ ID:585.

Another function of VGAM250 is therefore inhibition of Ribosomal Protein L39-like (RPL39L, Accession NM_{—}052969). Accordingly, utilities of VGAM250 include diagnosis, prevention and treatment of diseases and clinical conditions associated with RPL39L. Syntaphilin (SNPH, Accession NM_{—}014723) is another VGAM250 host target gene. SNPH BINDING SITE is HOST TARGET binding site found in the 3′ untranslated region of mRNA encoded by SNPH, corresponding to a HOST TARGET binding site such as BINDING SITE I, BINDING SITE II or BINDING SITE III. Table 2 illustrates the complementarity of the nucleotide sequences of SNPH BINDING SITE, designated SEQ ID:1531, to the nucleotide sequence of VGAM250 RNA, herein designated VGAM RNA, also designated SEQ ID:585.

Another function of VGAM250 is therefore inhibition of Syntaphilin (SNPH, Accession NM_{—}014723). Accordingly, utilities of VGAM250 include diagnosis, prevention and treatment of diseases and clinical conditions associated with SNPH. TMG3 (Accession NM_{—}024082) is another VGAM250 host target gene. TMG3 BINDING SITE is HOST TARGET binding site found in the 3′ untranslated region of mRNA encoded by TMG3, corresponding to a HOST TARGET binding site such as BINDING SITE I, BINDING SITE II or BINDING SITE III. Table 2 illustrates the complementarity of the nucleotide sequences of TMG3 BINDING SITE, designated SEQ ID:2053, to the nucleotide sequence of VGAM250 RNA, herein designated VGAM RNA, also designated SEQ ID:585.

Another function of VGAM250 is therefore inhibition of TMG3 (Accession NM_{—}024082). Accordingly, utilities of VGAM250 include diagnosis, prevention and treatment of diseases and clinical conditions associated with TMG3. LOC145240 (Accession XM_{—}085068) is another VGAM250 host target gene. LOC145240 BINDING SITE is HOST TARGET binding site found in the 3′ untranslated region of mRNA encoded by LOC145240, corresponding to a HOST TARGET binding site such as BINDING SITE I, BINDING SITE II or BINDING SITE III. Table 2 illustrates the complementarity of the nucleotide sequences of LOC145240 BINDING SITE, designated SEQ ID:3076, to the nucleotide sequence of VGAM250 RNA, herein designated VGAM RNA, also designated SEQ ID:585.

Another function of VGAM250 is therefore inhibition of LOC145240 (Accession XM_{—}085068). Accordingly, utilities of VGAM250 include diagnosis, prevention and treatment of diseases and clinical conditions associated with LOC145240. LOC146287 (Accession XM_{—}096967) is another VGAM250 host target gene. LOC146287 BINDING SITE is HOST TARGET binding site found in the 5′ untranslated region of mRNA encoded by LOC146287, corresponding to a HOST TARGET binding site such as BINDING SITE I, BINDING SITE II or BINDING SITE III. Table 2 illustrates the complementarity of the nucleotide sequences of LOC146287 BINDING SITE, designated SEQ ID:3284, to the nucleotide sequence of VGAM250 RNA, herein designated VGAM RNA, also designated SEQ ID:585.

Another function of VGAM250 is therefore inhibition of LOC146287 (Accession XM_{—}096967). Accordingly, utilities of VGAM250 include diagnosis, prevention and treatment of diseases and clinical conditions associated with LOC146287. LOC148696 (Accession XM_{—}097505) is another VGAM250 host target gene. LOC148696 BINDING SITE is HOST TARGET binding site found in the 5′ untranslated region of mRNA encoded by LOC148696, corresponding to a HOST TARGET binding site such as BINDING SITE I, BINDING SITE II or BINDING SITE III. Table 2 illustrates the complementarity of the nucleotide sequences of LOC148696 BINDING SITE, designated SEQ ID:3301, to the nucleotide sequence of VGAM250 RNA, herein designated VGAM RNA, also designated SEQ ID:585.

Another function of VGAM250 is therefore inhibition of LOC148696 (Accession XM_{—}097505). Accordingly, utilities of VGAM250 include diagnosis, prevention and treatment of diseases and clinical conditions associated with LOC148696. LOC150577 (Accession XM_{—}097918) is another VGAM250 host target gene. LOC150577 BINDING SITE is HOST TARGET binding site found in the 3′ untranslated region of mRNA encoded by LOC150577, corresponding to a HOST TARGET binding site such as BINDING SITE I, BINDING SITE II or BINDING SITE III. Table 2 illustrates the complementarity of the nucleotide sequences of LOC150577 BINDING SITE, designated SEQ ID:3330, to the nucleotide sequence of VGAM250 RNA, herein designated VGAM RNA, also designated SEQ ID:585.

Another function of VGAM250 is therefore inhibition of LOC150577 (Accession XM_{—}097918). Accordingly, utilities of VGAM250 include diagnosis, prevention and treatment of diseases and clinical conditions associated with LOC150577. LOC151521 (Accession XM_{—}098076) is another VGAM250 host target gene. LOC151521 BINDING SITE is HOST TARGET binding site found in the 3′ untranslated region of mRNA encoded by LOC151521, corresponding to a HOST TARGET binding site such as BINDING SITE I, BINDING SITE II or BINDING SITE III. Table 2 illustrates the complementarity of the nucleotide sequences of LOC151521 BINDING SITE, designated SEQ ID:3347, to the nucleotide sequence of VGAM250 RNA, herein designated VGAM RNA, also designated SEQ ID:585.

Another function of VGAM250 is therefore inhibition of LOC151521 (Accession XM_{—}098076). Accordingly, utilities of VGAM250 include diagnosis, prevention and treatment of diseases and clinical conditions associated with LOC151521. LOC157273 (Accession XM_{—}098743) is another VGAM250 host target gene. LOC157273 BINDING SITE is HOST TARGET binding site found in the 5′ untranslated region of mRNA encoded by LOC157273, corresponding to a HOST TARGET binding site such as BINDING SITE I, BINDING SITE II or BINDING SITE III. Table 2 illustrates the complementarity of the nucleotide sequences of LOC157273 BINDING SITE, designated SEQ ID:3379, to the nucleotide sequence of VGAM250 RNA, herein designated VGAM RNA, also designated SEQ ID:585.

Another function of VGAM250 is therefore inhibition of LOC157273 (Accession XM_{—}098743). Accordingly, utilities of VGAM250 include diagnosis, prevention and treatment of diseases and clinical conditions associated with LOC157273. LOC163682 (Accession XM_{—}099402) is another VGAM250 host target gene. LOC163682 BINDING SITE is HOST TARGET binding site found in the 5′ untranslated region of mRNA encoded by LOC163682, corresponding to a HOST TARGET binding site such as BINDING SITE I, BINDING SITE II or BINDING SITE III. Table 2 illustrates the complementarity of the nucleotide sequences of LOC163682 BINDING SITE, designated SEQ ID:3398, to the nucleotide sequence of VGAM250 RNA, herein designated VGAM RNA, also designated SEQ ID:585.

Another function of VGAM250 is therefore inhibition of LOC163682 (Accession XM_{—}099402). Accordingly, utilities of VGAM250 include diagnosis, prevention and treatment of diseases and clinical conditions associated with LOC163682. LOC221749 (Accession XM_{—}166341) is another VGAM250 host target gene. LOC221749 BINDING SITE is HOST TARGET binding site found in the 3′ untranslated region of mRNA encoded by LOC221749, corresponding to a HOST TARGET binding site such as BINDING SITE I, BINDING SITE II or BINDING SITE III. Table 2 illustrates the complementarity of the nucleotide sequences of LOC221749 BINDING SITE, designated SEQ ID:3549, to the nucleotide sequence of VGAM250 RNA, herein designated VGAM RNA, also designated SEQ ID:585.

Another function of VGAM250 is therefore inhibition of LOC221749 (Accession XM_{—}166341). Accordingly, utilities of VGAM250 include diagnosis, prevention and treatment of diseases and clinical conditions associated with LOC221749. LOC222234 (Accession XM_{—}168558) is another VGAM250 host target gene. LOC222234 BINDING SITE is HOST TARGET binding site found in the 3′ untranslated region of mRNA encoded by LOC222234, corresponding to a HOST TARGET binding site such as BINDING SITE I, BINDING SITE II or BINDING SITE III. Table 2 illustrates the complementarity of the nucleotide sequences of LOC222234 BINDING SITE, designated SEQ ID:3646, to the nucleotide sequence of VGAM250 RNA, herein designated VGAM RNA, also designated SEQ ID:585.

Another function of VGAM250 is therefore inhibition of LOC222234 (Accession XM_{—}168558). Accordingly, utilities of VGAM250 include diagnosis, prevention and treatment of diseases and clinical conditions associated with LOC222234. FIG. 1 further provides a conceptual description of a novel bioinformatically detected viral gene of the present invention, referred to here as Viral Genomic Address Messenger 251 (VGAM251) viral gene, which modulates expression of respective host target genes thereof, the function and utility of which host target genes is known in the art.

VGAM251 is a novel bioinformatically detected regulatory, non protein coding, viral micro RNA (miRNA) gene. The method by which VGAM251 was detected is described hereinabove with reference to FIGS. 1-8.

VGAM251 gene, herein designated VGAM GENE, is a viral gene contained in the genome of Vaccinia Virus. VGAM251 host target gene, herein designated VGAM HOST TARGET GENE, is a human gene contained in the human genome.

VGAM251 gene encodes a VGAM251 precursor RNA, herein designated VGAM PRECURSOR RNA. Similar to other miRNA genes, and unlike most ordinary genes, VGAM251 precursor RNA does not encode a protein. A nucleotide sequence identical or highly similar to the nucleotide sequence of VGAM251 precursor RNA is designated SEQ ID:237, and is provided hereinbelow with reference to the sequence listing part. Nucleotide sequence SEQ ID:237 is located at position 137709 relative to the genome of Vaccinia Virus.

VGAM251 precursor RNA folds onto itself, forming VGAM251 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM251 folded precursor RNA into VGAM251 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM251 RNA is designated SEQ ID:586, and is provided hereinbelow with reference to the sequence listing part.

VGAM251 host target gene, herein designated VGAM HOST TARGET GENE, encodes a corresponding messenger RNA, VGAM251 host target RNA, herein designated VGAM HOST TARGET RNA. VGAM251 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM251 RNA, herein designated VGAM RNA, binds complementarily to one or more host target binding sites located in untranslated regions of VGAM251 host target RNA, herein designated VGAM HOST TARGET RNA. This complementary binding is due to the fact that the nucleotide sequence of VGAM251 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM251 RNA, herein designated VGAM RNA, may have a different number of host target binding sites in untranslated regions of a VGAM251 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM251 RNA, herein designated VGAM RNA, to host target binding sites on VGAM251 host target RNA, herein designated VGAM HOST TARGET RNA, such as BINDING SITE I, BINDING SITE II and BINDING SITE III, inhibits translation of VGAM251 host target RNA into VGAM251 host target protein, herein designated VGAM HOST TARGET PROTEIN. VGAM host target protein is therefore outlined by a broken line.

It is appreciated that VGAM251 host target gene, herein designated VGAM HOST TARGET GENE, in fact represents a plurality of VGAM251 host target genes. The mRNA of each one of this plurality of VGAM251 host target genes comprises one or more host target binding sites, each having a nucleotide sequence which is at least partly complementary to VGAM251 RNA, herein designated VGAM RNA, and which when bound by VGAM251 RNA causes inhibition of translation of respective one or more VGAM251 host target proteins.

It is further appreciated by one skilled in the art that the mode of translational inhibition illustrated by FIG. 1 with specific reference to translational inhibition exerted by VGAM251 gene, herein designated VGAM GENE, on one or more VGAM251 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM251 is inhibition of expression of host target genes, as part of a novel viral mechanism of attacking a host. Accordingly, utilities of VGAM251 include diagnosis, prevention and treatment of viral infection by Vaccinia Virus. Specific functions, and accordingly utilities, of VGAM251 correlate with, and may be deduced from, the identity of the host target genes which VGAM251 binds and inhibits, and the function of these host target genes, as elaborated hereinbelow.

Nucleotide sequences of the VGAM251 precursor RNA, herein designated VGAM PRECURSOR RNA, and of the ‘diced’ VGAM251 RNA, herein designated VGAM RNA, and a schematic representation of the secondary folding of VGAM251 folded precursor RNA, herein designated VGAM FOLDED PRECURSOR RNA, of VGAM251 are further described hereinbelow with reference to Table 1.

Nucleotide sequences of host target binding sites, such as BINDING SITE-I, BINDING SITE-II and BINDING SITE-III of FIG. 1, found on VGAM251 host target RNA, and schematic representation of the complementarity of each of these host target binding sites to VGAM251 RNA, herein designated VGAM RNA, are described hereinbelow with reference to Table 2.

As mentioned hereinabove with reference to FIG. 1, a function of VGAM251 gene, herein designated VGAM is inhibition of expression of VGAM251 target genes. It is appreciated that specific functions, and accordingly utilities, of VGAM251 correlate with, and may be deduced from, the identity of the target genes which VGAM251 binds and inhibits, and the function of these target genes, as elaborated hereinbelow.

ADAMTS-like 1 (ADAMTSL1, Accession NM_{—}139264) is a VGAM251 host target gene. ADAMTSL1 BINDING SITE is HOST TARGET binding site found in the 3′ untranslated region of mRNA encoded by ADAMTSL1, corresponding to a HOST TARGET binding site such as BINDING SITE I, BINDING SITE II or BINDING SITE III. Table 2 illustrates the complementarity of the nucleotide sequences of ADAMTSL1 BINDING SITE, designated SEQ ID:2479, to the nucleotide sequence of VGAM251 RNA, herein designated VGAM RNA, also designated SEQ ID:586.

A function of VGAM251 is therefore inhibition of ADAMTS-like 1 (ADAMTSL1, Accession NM_{—}139264). Accordingly, utilities of VGAM251 include diagnosis, prevention and treatment of diseases and clinical conditions associated with ADAMTSL1. KIAA1862 (Accession XM_{—}044212) is another VGAM251 host target gene. KIAA1862 BINDING SITE is HOST TARGET binding site found in the 3′ untranslated region of mRNA encoded by KIAA1862, corresponding to a HOST TARGET binding site such as BINDING SITE I, BINDING SITE II or BINDING SITE III. Table 2 illustrates the complementarity of the nucleotide sequences of KIAA1862 BINDING SITE, designated SEQ ID:2834, to the nucleotide sequence of VGAM251 RNA, herein designated VGAM RNA, also designated SEQ ID:586.

Another function of VGAM251 is therefore inhibition of KIAA1862 (Accession XM_{—}044212). Accordingly, utilities of VGAM251 include diagnosis, prevention and treatment of diseases and clinical conditions associated with KIAA1862. LOC153951 (Accession XM_{—}087812) is another VGAM251 host target gene. LOC153951 BINDING SITE is HOST TARGET binding site found in the 5′ untranslated region of mRNA encoded by LOC153951, corresponding to a HOST TARGET binding site such as BINDING SITE I, BINDING SITE II or BINDING SITE III. Table 2 illustrates the complementarity of the nucleotide sequences of LOC153951 BINDING SITE, designated SEQ ID:3184, to the nucleotide sequence of VGAM251 RNA, herein designated VGAM RNA, also designated SEQ ID:586.

Another function of VGAM251 is therefore inhibition of LOC153951 (Accession XM_{—}087812). Accordingly, utilities of VGAM251 include diagnosis, prevention and treatment of diseases and clinical conditions associated with LOC153951. LOC221596 (Accession XM_{—}166331) is another VGAM251 host target gene. LOC221596 BINDING SITE is HOST TARGET binding site found in the 3′ untranslated region of mRNA encoded by LOC221596, corresponding to a HOST TARGET binding site such as BINDING SITE I, BINDING SITE II or BINDING SITE III. Table 2 illustrates the complementarity of the nucleotide sequences of LOC221596 BINDING SITE, designated SEQ ID:3548, to the nucleotide sequence of VGAM251 RNA, herein designated VGAM RNA, also designated SEQ ID:586.

Another function of VGAM251 is therefore inhibition of LOC221596 (Accession XM_{—}166331). Accordingly, utilities of VGAM251 include diagnosis, prevention and treatment of diseases and clinical conditions associated with LOC221596. FIG. 1 further provides a conceptual description of a novel bioinformatically detected viral gene of the present invention, referred to here as Viral Genomic Address Messenger 252 (VGAM252) viral gene, which modulates expression of respective host target genes thereof, the function and utility of which host target genes is known in the art.

VGAM252 is a novel bioinformatically detected regulatory, non protein coding, viral micro RNA (miRNA) gene. The method by which VGAM252 was detected is described hereinabove with reference to FIGS. 1-8.

VGAM252 gene, herein designated VGAM GENE, is a viral gene contained in the genome of Vaccinia Virus. VGAM252 host target gene, herein designated VGAM HOST TARGET GENE, is a human gene contained in the human genome.

VGAM252 gene encodes a VGAM252 precursor RNA, herein designated VGAM PRECURSOR RNA. Similar to other miRNA genes, and unlike most ordinary genes, VGAM252 precursor RNA does not encode a protein. A nucleotide sequence identical or highly similar to the nucleotide sequence of VGAM252 precursor RNA is designated SEQ ID:238, and is provided hereinbelow with reference to the sequence listing part. Nucleotide sequence SEQ ID:238 is located at position 140854 relative to the genome of Vaccinia Virus.

VGAM252 precursor RNA folds onto itself, forming VGAM252 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM252 folded precursor RNA into VGAM252 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM252 RNA is designated SEQ ID:587, and is provided hereinbelow with reference to the sequence listing part.

VGAM252 host target gene, herein designated VGAM HOST TARGET GENE, encodes a corresponding messenger RNA, VGAM252 host target RNA, herein designated VGAM HOST TARGET RNA. VGAM252 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM252 RNA, herein designated VGAM RNA, binds complementarily to one or more host target binding sites located in untranslated regions of VGAM252 host target RNA, herein designated VGAM HOST TARGET RNA. This complementary binding is due to the fact that the nucleotide sequence of VGAM252 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM252 RNA, herein designated VGAM RNA, may have a different number of host target binding sites in untranslated regions of a VGAM252 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM252 RNA, herein designated VGAM RNA, to host target binding sites on VGAM252 host target RNA, herein designated VGAM HOST TARGET RNA, such as BINDING SITE I, BINDING SITE II and BINDING SITE III, inhibits translation of VGAM252 host target RNA into VGAM252 host target protein, herein designated VGAM HOST TARGET PROTEIN. VGAM host target protein is therefore outlined by a broken line.

It is appreciated that VGAM252 host target gene, herein designated VGAM HOST TARGET GENE, in fact represents a plurality of VGAM252 host target genes. The mRNA of each one of this plurality of VGAM252 host target genes comprises one or more host target binding sites, each having a nucleotide sequence which is at least partly complementary to VGAM252 RNA, herein designated VGAM RNA, and which when bound by VGAM252 RNA causes inhibition of translation of respective one or more VGAM252 host target proteins.

It is further appreciated by one skilled in the art that the mode of translational inhibition illustrated by FIG. 1 with specific reference to translational inhibition exerted by VGAM252 gene, herein designated VGAM GENE, on one or more VGAM252 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM252 is inhibition of expression of host target genes, as part of a novel viral mechanism of attacking a host. Accordingly, utilities of VGAM252 include diagnosis, prevention and treatment of viral infection by Vaccinia Virus. Specific functions, and accordingly utilities, of VGAM252 correlate with, and may be deduced from, the identity of the host target genes which VGAM252 binds and inhibits, and the function of these host target genes, as elaborated hereinbelow.

Nucleotide sequences of the VGAM252 precursor RNA, herein designated VGAM PRECURSOR RNA, and of the ‘diced’ VGAM252 RNA, herein designated VGAM RNA, and a schematic representation of the secondary folding of VGAM252 folded precursor RNA, herein designated VGAM FOLDED PRECURSOR RNA, of VGAM252 are further described hereinbelow with reference to Table 1.

Nucleotide sequences of host target binding sites, such as BINDING SITE-I, BINDING SITE-II and BINDING SITE-III of FIG. 1, found on VGAM252 host target RNA, and schematic representation of the complementarity of each of these host target binding sites to VGAM252 RNA, herein designated VGAM RNA, are described hereinbelow with reference to Table 2.

As mentioned hereinabove with reference to FIG. 1, a function of VGAM252 gene, herein designated VGAM is inhibition of expression of VGAM252 target genes. It is appreciated that specific functions, and accordingly utilities, of VGAM252 correlate with, and may be deduced from, the identity of the target genes which VGAM252 binds and inhibits, and the function of these target genes, as elaborated hereinbelow.

ESAM (Accession NM_{—}138961) is a VGAM252 host target gene. ESAM BINDING SITE is HOST TARGET binding site found in the 3′ untranslated region of mRNA encoded by ESAM, corresponding to a HOST TARGET binding site such as BINDING SITE I, BINDING SITE II or BINDING SITE III. Table 2 illustrates the complementarity of the nucleotide sequences of ESAM BINDING SITE, designated SEQ ID:2461, to the nucleotide sequence of VGAM252 RNA, herein designated VGAM RNA, also designated SEQ ID:587.

A function of VGAM252 is therefore inhibition of ESAM (Accession NM_{—}138961). Accordingly, utilities of VGAM252 include diagnosis, prevention and treatment of diseases and clinical conditions associated with ESAM. KIAA0379 (Accession XM_{—}042860) is another VGAM252 host target gene. KIAA0379 BINDING SITE is HOST TARGET binding site found in the 3′ untranslated region of mRNA encoded by KIAA0379, corresponding to a HOST TARGET binding site such as BINDING SITE I, BINDING SITE II or BINDING SITE III. Table 2 illustrates the complementarity of the nucleotide sequences of KIAA0379 BINDING SITE, designated SEQ ID:2809, to the nucleotide sequence of VGAM252 RNA, herein designated VGAM RNA, also designated SEQ ID:587.

Another function of VGAM252 is therefore inhibition of KIAA0379 (Accession XM_{—}042860). Accordingly, utilities of VGAM252 include diagnosis, prevention and treatment of diseases and clinical conditions associated with KIAA0379. LOC161784 (Accession XM_{—}091123) is another VGAM252 host target gene. LOC161784 BINDING SITE is HOST TARGET binding site found in the 3′ untranslated region of mRNA encoded by LOC161784, corresponding to a HOST TARGET binding site such as BINDING SITE I, BINDING SITE II or BINDING SITE III. Table 2 illustrates the complementarity of the nucleotide sequences of LOC161784 BINDING SITE, designated SEQ ID:3241, to the nucleotide sequence of VGAM252 RNA, herein designated VGAM RNA, also designated SEQ ID:587.

Another function of VGAM252 is therefore inhibition of LOC161784 (Accession XM_{—}091123). Accordingly, utilities of VGAM252 include diagnosis, prevention and treatment of diseases and clinical conditions associated with LOC161784. LOC255229 (Accession XM_{—}170928) is another VGAM252 host target gene. LOC255229 BINDING SITE is HOST TARGET binding site found in the 3′ untranslated region of mRNA encoded by LOC255229, corresponding to a HOST TARGET binding site such as BINDING SITE I, BINDING SITE II or BINDING SITE III. Table 2 illustrates the complementarity of the nucleotide sequences of LOC255229 BINDING SITE, designated SEQ ID:3676, to the nucleotide sequence of VGAM252 RNA, herein designated VGAM RNA, also designated SEQ ID:587.

Another function of VGAM252 is therefore inhibition of LOC255229 (Accession XM_{—}170928). Accordingly, utilities of VGAM252 include diagnosis, prevention and treatment of diseases and clinical conditions associated with LOC255229. FIG. 1 further provides a conceptual description of a novel bioinformatically detected viral gene of the present invention, referred to here as Viral Genomic Address Messenger 253 (VGAM253) viral gene, which modulates expression of respective host target genes thereof, the function and utility of which host target genes is known in the art.

VGAM253 is a novel bioinformatically detected regulatory, non protein coding, viral micro RNA (miRNA) gene. The method by which VGAM253 was detected is described hereinabove with reference to FIGS. 1-8.

VGAM253 gene, herein designated VGAM GENE, is a viral gene contained in the genome of Vaccinia Virus. VGAM253 host target gene, herein designated VGAM HOST TARGET GENE, is a human gene contained in the human genome.

VGAM253 gene encodes a VGAM253 precursor RNA, herein designated VGAM PRECURSOR RNA. Similar to other miRNA genes, and unlike most ordinary genes, VGAM253 precursor RNA does not encode a protein. A nucleotide sequence identical or highly similar to the nucleotide sequence of VGAM253 precursor RNA is designated SEQ ID:239, and is provided hereinbelow with reference to the sequence listing part. Nucleotide sequence SEQ ID:239 is located at position 140990 relative to the genome of Vaccinia Virus.

VGAM253 precursor RNA folds onto itself, forming VGAM253 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM253 folded precursor RNA into VGAM253 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM253 RNA is designated SEQ ID:588, and is provided hereinbelow with reference to the sequence listing part.

VGAM253 host target gene, herein designated VGAM HOST TARGET GENE, encodes a corresponding messenger RNA, VGAM253 host target RNA, herein designated VGAM HOST TARGET RNA. VGAM253 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM253 RNA, herein designated VGAM RNA, binds complementarily to one or more host target binding sites located in untranslated regions of VGAM253 host target RNA, herein designated VGAM HOST TARGET RNA. This complementary binding is due to the fact that the nucleotide sequence of VGAM253 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM253 RNA, herein designated VGAM RNA, may have a different number of host target binding sites in untranslated regions of a VGAM253 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM253 RNA, herein designated VGAM RNA, to host target binding sites on VGAM253 host target RNA, herein designated VGAM HOST TARGET RNA, such as BINDING SITE I, BINDING SITE II and BINDING SITE III, inhibits translation of VGAM253 host target RNA into VGAM253 host target protein, herein designated VGAM HOST TARGET PROTEIN. VGAM host target protein is therefore outlined by a broken line.

It is appreciated that VGAM253 host target gene, herein designated VGAM HOST TARGET GENE, in fact represents a plurality of VGAM253 host target genes. The mRNA of each one of this plurality of VGAM253 host target genes comprises one or more host target binding sites, each having a nucleotide sequence which is at least partly complementary to VGAM253 RNA, herein designated VGAM RNA, and which when bound by VGAM253 RNA causes inhibition of translation of respective one or more VGAM253 host target proteins.

It is further appreciated by one skilled in the art that the mode of translational inhibition illustrated by FIG. 1 with specific reference to translational inhibition exerted by VGAM253 gene, herein designated VGAM GENE, on one or more VGAM253 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM253 is inhibition of expression of host target genes, as part of a novel viral mechanism of attacking a host. Accordingly, utilities of VGAM253 include diagnosis, prevention and treatment of viral infection by Vaccinia Virus. Specific functions, and accordingly utilities, of VGAM253 correlate with, and may be deduced from, the identity of the host target genes which VGAM253 binds and inhibits, and the function of these host target genes, as elaborated hereinbelow.

Nucleotide sequences of the VGAM253 precursor RNA, herein designated VGAM PRECURSOR RNA, and of the ‘diced’ VGAM253 RNA, herein designated VGAM RNA, and a schematic representation of the secondary folding of VGAM253 folded precursor RNA, herein designated VGAM FOLDED PRECURSOR RNA, of VGAM253 are further described hereinbelow with reference to Table 1.

Nucleotide sequences of host target binding sites, such as BINDING SITE-I, BINDING SITE-II and BINDING SITE-III of FIG. 1, found on VGAM253 host target RNA, and schematic representation of the complementarity of each of these host target binding sites to VGAM253 RNA, herein designated VGAM RNA, are described hereinbelow with reference to Table 2.

As mentioned hereinabove with reference to FIG. 1, a function of VGAM253 gene, herein designated VGAM is inhibition of expression of VGAM253 target genes. It is appreciated that specific functions, and accordingly utilities, of VGAM253 correlate with, and may be deduced from, the identity of the target genes which VGAM253 binds and inhibits, and the function of these target genes, as elaborated hereinbelow.

Huntingtin Interacting Protein 2 (HIP2, Accession NM_{—}005339) is a VGAM253 host target gene. HIP2 BINDING SITE is HOST TARGET binding site found in the 3′ untranslated region of mRNA encoded by HIP2, corresponding to a HOST TARGET binding site such as BINDING SITE I, BINDING SITE II or BINDING SITE III. Table 2 illustrates the complementarity of the nucleotide sequences of HIP2 BINDING SITE, designated SEQ ID:1201, to the nucleotide sequence of VGAM253 RNA, herein designated VGAM RNA, also designated SEQ ID:588.

A function of VGAM253 is therefore inhibition of Huntingtin Interacting Protein 2 (HIP2, Accession NM_{—}005339). Accordingly, utilities of VGAM253 include diagnosis, prevention and treatment of diseases and clinical conditions associated with HIP2. IL2-inducible T-cell Kinase (ITK, Accession NM_{—}005546) is another VGAM253 host target gene. ITK BINDING SITE is HOST TARGET binding site found in the 3′ untranslated region of mRNA encoded by ITK, corresponding to a HOST TARGET binding site such as BINDING SITE I, BINDING SITE II or BINDING SITE III. Table 2 illustrates the complementarity of the nucleotide sequences of ITK BINDING SITE, designated SEQ ID:1224, to the nucleotide sequence of VGAM253 RNA, herein designated VGAM RNA, also designated SEQ ID:588.

Another function of VGAM253 is therefore inhibition of IL2-inducible T-cell Kinase (ITK, Accession NM_{—}005546), a gene which plays a role in t cell proliferation and differentiation. Accordingly, utilities of VGAM253 include diagnosis, prevention and treatment of diseases and clinical conditions associated with ITK. The function of ITK and its association with various diseases and clinical conditions, has been established by previous studies, as described hereinabove with reference to VGAM191. One Cut Domain, Family Member 1 (ONECUT1, Accession XM_{—}030712) is another VGAM253 host target gene. ONECUT1 BINDING SITE is HOST TARGET binding site found in the 3′ untranslated region of mRNA encoded by ONECUT1, corresponding to a HOST TARGET binding site such as BINDING SITE I, BINDING SITE II or BINDING SITE III. Table 2 illustrates the complementarity of the nucleotide sequences of ONECUT1 BINDING SITE, designated SEQ ID:2622, to the nucleotide sequence of VGAM253 RNA, herein designated VGAM RNA, also designated SEQ ID:588.

Another function of VGAM253 is therefore inhibition of One Cut Domain, Family Member 1 (ONECUT1, Accession XM_{—}030712), a gene which is very strongly similar to murine Hnf6 and may regulate transcription of liver-specific genes. Accordingly, utilities of VGAM253 include diagnosis, prevention and treatment of diseases and clinical conditions associated with ONECUT1. The function of ONECUT1 has been established by previous studies. Lemaigre et al. (1996) isolated from rat liver a transcription factor, which they called hepatocyte nuclear factor-6, that contains 2 different DNA-binding domains: a novel type of homeodomain, and a homolog of the Drosophila cut domain. A similar bipartite sequence had been found only in the genome of Caenorhabditis elegans. The HNF6 cDNA encodes a protein of 465 amino acids. By RNase protection assay, the expression of HNF6 was found to be highest in liver, but was also detectable in brain, spleen, and testis. Animal model experiments lend further support to the function of ONECUT1. During mouse development, Hnf6 is expressed in the epithelial cells that are precursors of the exocrine and endocrine pancreatic cells. Jacquemin et al. (2000) investigated the role of Hnf6 in pancreas differentiation by inactivating its gene in the mouse. In hnf6-null embryos, the exocrine pancreas appeared to be normal but endocrine cell differentiation was impaired. The expression of neurogenin-3 (NGN3; 604882), a transcription factor that is essential for determination of endocrine cell precursors, was almost abolished. Consistent with this, Jacquemin et al. (2000) demonstrated that Hnf6 binds to and stimulates the ngn3 gene promoter. At birth, only a few endocrine cells were found and the islets of Langerhans were missing. Later, the number of endocrine cells increased and islets appeared. However, the architecture of the islets was perturbed, and the beta cells were deficient in glucose transporter-2 (SLC2A2; 138160) expression. Adult hnf6-null mice were diabetic. The authors concluded that Hnf6 controls pancreatic endocrine differentiation at the precursor stage and stated that their data identify Hnf6 as the first positive regulator of the proendocrine gene ngn3 in the pancreas.

It is appreciated that the abovementioned animal model for ONECUT1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Jacquemin, P.; Durviaux, S. M.; Jensen, J.; Godfraind, C.; Gradwohl, G.; Guillemot, F.; Madsen, O. D.; Carmeliet, P.; Dewerchin, M.; Collen, D.; Rousseau, G. G.; Lemaigre, F. P.: Transcription factor hepatocyte nuclear factor 6 regulates pancreatic endocrine cell differentiation and controls expression of the proendocrine gene ngn3. Molec. Cell. Biol. 20: 4445-4454, 2000.; and
Lemaigre, F. P.; Durviaux, S. M.; Truong, O.; Lannoy, V. J.; Hsuan, J. J.; Rousseau, G. G.: Hepatocyte nuclear factor 6, a transcription factor that contains a novel type of homeodom.

Further studies establishing the function and utilities of ONECUT1 are found in John Hopkins OMIM database record ID 604164, and in sited publications numbered 1807-1812 listed in the bibliography section hereinbelow, which are also hereby incorporated by reference. Butyrophilin, Sub family 3, Member A2 (BTN3A2, Accession NM_{—}007047) is another VGAM253 host target gene. BTN3A2 BINDING SITE is HOST TARGET binding site found in the 5′ untranslated region of mRNA encoded by BTN3A2, corresponding to a HOST TARGET binding site such as BINDING SITE I, BINDING SITE II or BINDING SITE III. Table 2 illustrates the complementarity of the nucleotide sequences of BTN3A2 BINDING SITE, designated SEQ ID:1354, to the nucleotide sequence of VGAM253 RNA, herein designated VGAM RNA, also designated SEQ ID:588.

Another function of VGAM253 is therefore inhibition of Butyrophilin, Sub family 3, Member A2 (BTN3A2, Accession NM_{—}007047). Accordingly, utilities of VGAM253 include diagnosis, prevention and treatment of diseases and clinical conditions associated with BTN3A2. Butyrophilin, Sub family 3, Member A3 (BTN3A3, Accession NM_{—}006994) is another VGAM253 host target gene. BTN3A3 BINDING SITE is HOST TARGET binding site found in the 5′ untranslated region of mRNA encoded by BTN3A3, corresponding to a HOST TARGET binding site such as BINDING SITE I, BINDING SITE II or BINDING SITE III. Table 2 illustrates the complementarity of the nucleotide sequences of BTN3A3 BINDING SITE, designated SEQ ID:1343, to the nucleotide sequence of VGAM253 RNA, herein designated VGAM RNA, also designated SEQ ID:588.

Another function of VGAM253 is therefore inhibition of Butyrophilin, Sub family 3, Member A3 (BTN3A3, Accession NM_{—}006994). Accordingly, utilities of VGAM253 include diagnosis, prevention and treatment of diseases and clinical conditions associated with BTN3A3. IDN3 (Accession NM_{—}133433) is another VGAM253 host target gene. IDN3 BINDING SITE is HOST TARGET binding site found in the 3′ untranslated region of mRNA encoded by IDN3, corresponding to a HOST TARGET binding site such as BINDING SITE I, BINDING SITE II or BINDING SITE III. Table 2 illustrates the complementarity of the nucleotide sequences of IDN3 BINDING SITE, designated SEQ ID:2416, to the nucleotide sequence of VGAM253 RNA, herein designated VGAM RNA, also designated SEQ ID:588.

Another function of VGAM253 is therefore inhibition of IDN3 (Accession NM_{—}133433). Accordingly, utilities of VGAM253 include diagnosis, prevention and treatment of diseases and clinical conditions associated with IDN3. KIAA1635 (Accession XM_{—}042203) is another VGAM253 host target gene. KIAA1635 BINDING SITE is HOST TARGET binding site found in the 3′ untranslated region of mRNA encoded by KIAA1635, corresponding to a HOST TARGET binding site such as BINDING SITE I, BINDING SITE II or BINDING SITE III. Table 2 illustrates the complementarity of the nucleotide sequences of KIAA1635 BINDING SITE, designated SEQ ID:2803, to the nucleotide sequence of VGAM253 RNA, herein designated VGAM RNA, also designated SEQ ID:588.

Another function of VGAM253 is therefore inhibition of KIAA1635 (Accession XM_{—}042203). Accordingly, utilities of VGAM253 include diagnosis, prevention and treatment of diseases and clinical conditions associated with KIAA1635. LOC130106 (Accession XM_{—}059399) is another VGAM253 host target gene. LOC130106 BINDING SITE is HOST TARGET binding site found in the 3′ untranslated region of mRNA encoded by LOC130106, corresponding to a HOST TARGET binding site such as BINDING SITE I, BINDING SITE II or BINDING SITE III. Table 2 illustrates the complementarity of the nucleotide sequences of LOC130106 BINDING SITE, designated SEQ ID:3008, to the nucleotide sequence of VGAM253 RNA, herein designated VGAM RNA, also designated SEQ ID:588.

Another function of VGAM253 is therefore inhibition of LOC130106 (Accession XM_{—}059399). Accordingly, utilities of VGAM253 include diagnosis, prevention and treatment of diseases and clinical conditions associated with LOC130106. LOC151031 (Accession XM_{—}103784) is another VGAM253 host target gene. LOC151031 BINDING SITE is HOST TARGET binding site found in the 3′ untranslated region of mRNA encoded by LOC151031, corresponding to a HOST TARGET binding site such as BINDING SITE I, BINDING SITE II or BINDING SITE III. Table 2 illustrates the complementarity of the nucleotide sequences of LOC151031 BINDING SITE, designated SEQ ID:3403, to the nucleotide sequence of VGAM253 RNA, herein designated VGAM RNA, also designated SEQ ID:588.

Another function of VGAM253 is therefore inhibition of LOC151031 (Accession XM_{—}103784). Accordingly, utilities of VGAM253 include diagnosis, prevention and treatment of diseases and clinical conditions associated with LOC151031. LOC162545 (Accession XM_{—}102745) is another VGAM253 host target gene. LOC162545 BINDING SITE is HOST TARGET binding site found in the 3′ untranslated region of mRNA encoded by LOC162545, corresponding to a HOST TARGET binding site such as BINDING SITE I, BINDING SITE II or BINDING SITE III. Table 2 illustrates the complementarity of the nucleotide sequences of LOC162545 BINDING SITE, designated SEQ ID:3402, to the nucleotide sequence of VGAM253 RNA, herein designated VGAM RNA, also designated SEQ ID:588.

Another function of VGAM253 is therefore inhibition of LOC162545 (Accession XM_{—}102745). Accordingly, utilities of VGAM253 include diagnosis, prevention and treatment of diseases and clinical conditions associated with LOC162545. LOC165666 (Accession XM_{—}093359) is another VGAM253 host target gene. LOC165666 BINDING SITE is HOST TARGET binding site found in the 5′ untranslated region of mRNA encoded by LOC165666, corresponding to a HOST TARGET binding site such as BINDING SITE I, BINDING SITE II or BINDING SITE III. Table 2 illustrates the complementarity of the nucleotide sequences of LOC165666 BINDING SITE, designated SEQ ID:3257, to the nucleotide sequence of VGAM253 RNA, herein designated VGAM RNA, also designated SEQ ID:588.

Another function of VGAM253 is therefore inhibition of LOC165666 (Accession XM_{—}093359). Accordingly, utilities of VGAM253 include diagnosis, prevention and treatment of diseases and clinical conditions associated with LOC165666. FIG. 1 further provides a conceptual description of a novel bioinformatically detected viral gene of the present invention, referred to here as Viral Genomic Address Messenger 254 (VGAM254) viral gene, which modulates expression of respective host target genes thereof, the function and utility of which host target genes is known in the art.

VGAM254 is a novel bioinformatically detected regulatory, non protein coding, viral micro RNA (miRNA) gene. The method by which VGAM254 was detected is described hereinabove with reference to FIGS. 1-8.

VGAM254 gene, herein designated VGAM GENE, is a viral gene contained in the genome of Vaccinia Virus. VGAM254 host target gene, herein designated VGAM HOST TARGET GENE, is a human gene contained in the human genome.

VGAM254 gene encodes a VGAM254 precursor RNA, herein designated VGAM PRECURSOR RNA. Similar to other miRNA genes, and unlike most ordinary genes, VGAM254 precursor RNA does not encode a protein. A nucleotide sequence identical or highly similar to the nucleotide sequence of VGAM254 precursor RNA is designated SEQ ID:240, and is provided hereinbelow with reference to the sequence listing part. Nucleotide sequence SEQ ID:240 is located at position 141210 relative to the genome of Vaccinia Virus.

VGAM254 precursor RNA folds onto itself, forming VGAM254 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM254 folded precursor RNA into VGAM254 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM254 RNA is designated SEQ ID:589, and is provided hereinbelow with reference to the sequence listing part.

VGAM254 host target gene, herein designated VGAM HOST TARGET GENE, encodes a corresponding messenger RNA, VGAM254 host target RNA, herein designated VGAM HOST TARGET RNA. VGAM254 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM254 RNA, herein designated VGAM RNA, binds complementarily to one or more host target binding sites located in untranslated regions of VGAM254 host target RNA, herein designated VGAM HOST TARGET RNA. This complementary binding is due to the fact that the nucleotide sequence of VGAM254 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM254 RNA, herein designated VGAM RNA, may have a different number of host target binding sites in untranslated regions of a VGAM254 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM254 RNA, herein designated VGAM RNA, to host target binding sites on VGAM254 host target RNA, herein designated VGAM HOST TARGET RNA, such as BINDING SITE I, BINDING SITE II and BINDING SITE III, inhibits translation of VGAM254 host target RNA into VGAM254 host target protein, herein designated VGAM HOST TARGET PROTEIN. VGAM host target protein is therefore outlined by a broken line.

It is appreciated that VGAM254 host target gene, herein designated VGAM HOST TARGET GENE, in fact represents a plurality of VGAM254 host target genes. The mRNA of each one of this plurality of VGAM254 host target genes comprises one or more host target binding sites, each having a nucleotide sequence which is at least partly complementary to VGAM254 RNA, herein designated VGAM RNA, and which when bound by VGAM254 RNA causes inhibition of translation of respective one or more VGAM254 host target proteins.

It is further appreciated by one skilled in the art that the mode of translational inhibition illustrated by FIG. 1 with specific reference to translational inhibition exerted by VGAM254 gene, herein designated VGAM GENE, on one or more VGAM254 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM254 is inhibition of expression of host target genes, as part of a novel viral mechanism of attacking a host. Accordingly, utilities of VGAM254 include diagnosis, prevention and treatment of viral infection by Vaccinia Virus. Specific functions, and accordingly utilities, of VGAM254 correlate with, and may be deduced from, the identity of the host target genes which VGAM254 binds and inhibits, and the function of these host target genes, as elaborated hereinbelow.

Nucleotide sequences of the VGAM254 precursor RNA, herein designated VGAM PRECURSOR RNA, and of the ‘diced’ VGAM254 RNA, herein designated VGAM RNA, and a schematic representation of the secondary folding of VGAM254 folded precursor RNA, herein designated VGAM FOLDED PRECURSOR RNA, of VGAM254 are further described hereinbelow with reference to Table 1.

Nucleotide sequences of host target binding sites, such as BINDING SITE-I, BINDING SITE-II and BINDING SITE-III of FIG. 1, found on VGAM254 host target RNA, and schematic representation of the complementarity of each of these host target binding sites to VGAM254 RNA, herein designated VGAM RNA, are described hereinbelow with reference to Table 2.

As mentioned hereinabove with reference to FIG. 1, a function of VGAM254 gene, herein designated VGAM is inhibition of expression of VGAM254 target genes. It is appreciated that specific functions, and accordingly utilities, of VGAM254 correlate with, and may be deduced from, the identity of the target genes which VGAM254 binds and inhibits, and the function of these target genes, as elaborated hereinbelow.

Multiple Endocrine Neoplasia I (MEN1, Accession XM_{—}167804) is a VGAM254 host target gene. MEN1 BINDING SITE is HOST TARGET binding site found in the 5′ untranslated region of mRNA encoded by MEN1, corresponding to a HOST TARGET binding site such as BINDING SITE I, BINDING SITE II or BINDING SITE III. Table 2 illustrates the complementarity of the nucleotide sequences of MEN1 BINDING SITE, designated SEQ ID:3599, to the nucleotide sequence of VGAM254 RNA, herein designated VGAM RNA, also designated SEQ ID:589.

A function of VGAM254 is therefore inhibition of Multiple Endocrine Neoplasia I (MEN1, Accession XM_{—}167804). Accordingly, utilities of VGAM254 include diagnosis, prevention and treatment of diseases and clinical conditions associated with MEN1. Steroid-5-alpha-reductase, Alpha Polypeptide 2 (3-oxo-5 alpha-steroid delta 4-dehydrogenase alpha 2) (SRD5A2, Accession XM_{—}002471) is another VGAM254 host target gene. SRD5A2 BINDING SITE is HOST TARGET binding site found in the 5′ untranslated region of mRNA encoded by SRD5A2, corresponding to a HOST TARGET binding site such as BINDING SITE I, BINDING SITE II or BINDING SITE III. Table 2 illustrates the complementarity of the nucleotide sequences of SRD5A2 BINDING SITE, designated SEQ ID:2525, to the nucleotide sequence of VGAM254 RNA, herein designated VGAM RNA, also designated SEQ ID:589.

Another function of VGAM254 is therefore inhibition of Steroid-5-alpha-reductase, Alpha Polypeptide 2 (3-oxo-5 alpha-steroid delta 4-dehydrogenase alpha 2) (SRD5A2, Accession XM_{—}002471). Accordingly, utilities of VGAM254 include diagnosis, prevention and treatment of diseases and clinical conditions associated with SRD5A2. DKFZP5640043 (Accession XM_{—}166502) is another VGAM254 host target gene. DKFZP5640043 BINDING SITE is HOST TARGET binding site found in the 3′ untranslated region of mRNA encoded by DKFZP5640043, corresponding to a HOST TARGET binding site such as BINDING SITE I, BINDING SITE II or BINDING SITE III. Table 2 illustrates the complementarity of the nucleotide sequences of DKFZP5640043 BINDING SITE, designated SEQ ID:3566, to the nucleotide sequence of VGAM254 RNA, herein designated VGAM RNA, also designated SEQ ID:589.

Another function of VGAM254 is therefore inhibition of DKFZP5640043 (Accession XM_{—}166502). Accordingly, utilities of VGAM254 include diagnosis, prevention and treatment of diseases and clinical conditions associated with DKFZP5640043. FLJ22794 (Accession XM_{—}166220) is another VGAM254 host target gene. FLJ22794 BINDING SITE is HOST TARGET binding site found in the 5′ untranslated region of mRNA encoded by FLJ22794, corresponding to a HOST TARGET binding site such as BINDING SITE I, BINDING SITE II or BINDING SITE III. Table 2 illustrates the complementarity of the nucleotide sequences of FLJ22794 BINDING SITE, designated SEQ ID:3533, to the nucleotide sequence of VGAM254 RNA, herein designated VGAM RNA, also designated SEQ ID:589.

Another function of VGAM254 is therefore inhibition of FLJ22794 (Accession XM_{—}166220). Accordingly, utilities of VGAM254 include diagnosis, prevention and treatment of diseases and clinical conditions associated with FLJ22794. HTMP10 (Accession NM_{—}033207) is another VGAM254 host target gene. HTMP10 BINDING SITE is HOST TARGET binding site found in the 3′ untranslated region of mRNA encoded by HTMP10, corresponding to a HOST TARGET binding site such as BINDING SITE I, BINDING SITE II or BINDING SITE III. Table 2 illustrates the complementarity of the nucleotide sequences of HTMP10 BINDING SITE, designated SEQ ID:2322, to the nucleotide sequence of VGAM254 RNA, herein designated VGAM RNA, also designated SEQ ID:589.

Another function of VGAM254 is therefore inhibition of HTMP10 (Accession NM_{—}033207). Accordingly, utilities of VGAM254 include diagnosis, prevention and treatment of diseases and clinical conditions associated with HTMP10. SEC24 Related Gene Family, Member B (S. cerevisiae) (SEC24B, Accession NM_{—}006323) is another VGAM254 host target gene. SEC24B BINDING SITE is HOST TARGET binding site found in the 3′ untranslated region of mRNA encoded by SEC24B, corresponding to a HOST TARGET binding site such as BINDING SITE I, BINDING SITE II or BINDING SITE III. Table 2 illustrates the complementarity of the nucleotide sequences of SEC24B BINDING SITE, designated SEQ ID:1288, to the nucleotide sequence of VGAM254 RNA, herein designated VGAM RNA, also designated SEQ ID:589.

Another function of VGAM254 is therefore inhibition of SEC24 Related Gene Family, Member B (S. cerevisiae) (SEC24B, Accession NM_{—}006323). Accordingly, utilities of VGAM254 include diagnosis, prevention and treatment of diseases and clinical conditions associated with SEC24B. LOC196812 (Accession XM_{—}116868) is another VGAM254 host target gene. LOC196812 BINDING SITE is HOST TARGET binding site found in the 5′ untranslated region of mRNA encoded by LOC196812, corresponding to a HOST TARGET binding site such as BINDING SITE I, BINDING SITE II or BINDING SITE III. Table 2 illustrates the complementarity of the nucleotide sequences of LOC196812 BINDING SITE, designated SEQ ID:3466, to the nucleotide sequence of VGAM254 RNA, herein designated VGAM RNA, also designated SEQ ID:589.

Another function of VGAM254 is therefore inhibition of LOC196812 (Accession XM_{—}116868). Accordingly, utilities of VGAM254 include diagnosis, prevention and treatment of diseases and clinical conditions associated with LOC196812. FIG. 1 further provides a conceptual description of a novel bioinformatically detected viral gene of the present invention, referred to here as Viral Genomic Address Messenger 255 (VGAM255) viral gene, which modulates expression of respective host target genes thereof, the function and utility of which host target genes is known in the art.

VGAM255 is a novel bioinformatically detected regulatory, non protein coding, viral micro RNA (miRNA) gene. The method by which VGAM255 was detected is described hereinabove with reference to FIGS. 1-8.

VGAM255 gene, herein designated VGAM GENE, is a viral gene contained in the genome of Vaccinia Virus. VGAM255 host target gene, herein designated VGAM HOST TARGET GENE, is a human gene contained in the human genome.

VGAM255 gene encodes a VGAM255 precursor RNA, herein designated VGAM PRECURSOR RNA. Similar to other miRNA genes, and unlike most ordinary genes, VGAM255 precursor RNA does not encode a protein. A nucleotide sequence identical or highly similar to the nucleotide sequence of VGAM255 precursor RNA is designated SEQ ID:241, and is provided hereinbelow with reference to the sequence listing part. Nucleotide sequence SEQ ID:241 is located at position 141044 relative to the genome of Vaccinia Virus.

VGAM255 precursor RNA folds onto itself, forming VGAM255 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM255 folded precursor RNA into VGAM255 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM255 RNA is designated SEQ ID:590, and is provided hereinbelow with reference to the sequence listing part.

VGAM255 host target gene, herein designated VGAM HOST TARGET GENE, encodes a corresponding messenger RNA, VGAM255 host target RNA, herein designated VGAM HOST TARGET RNA. VGAM255 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM255 RNA, herein designated VGAM RNA, binds complementarily to one or more host target binding sites located in untranslated regions of VGAM255 host target RNA, herein designated VGAM HOST TARGET RNA. This complementary binding is due to the fact that the nucleotide sequence of VGAM255 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM255 RNA, herein designated VGAM RNA, may have a different number of host target binding sites in untranslated regions of a VGAM255 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM255 RNA, herein designated VGAM RNA, to host target binding sites on VGAM255 host target RNA, herein designated VGAM HOST TARGET RNA, such as BINDING SITE I, BINDING SITE II and BINDING SITE III, inhibits translation of VGAM255 host target RNA into VGAM255 host target protein, herein designated VGAM HOST TARGET PROTEIN. VGAM host target protein is therefore outlined by a broken line.

It is appreciated that VGAM255 host target gene, herein designated VGAM HOST TARGET GENE, in fact represents a plurality of VGAM255 host target genes. The mRNA of each one of this plurality of VGAM255 host target genes comprises one or more host target binding sites, each having a nucleotide sequence which is at least partly complementary to VGAM255 RNA, herein designated VGAM RNA, and which when bound by VGAM255 RNA causes inhibition of translation of respective one or more VGAM255 host target proteins.

It is further appreciated by one skilled in the art that the mode of translational inhibition illustrated by FIG. 1 with specific reference to translational inhibition exerted by VGAM255 gene, herein designated VGAM GENE, on one or more VGAM255 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM255 is inhibition of expression of host target genes, as part of a novel viral mechanism of attacking a host. Accordingly, utilities of VGAM255 include diagnosis, prevention and treatment of viral infection by Vaccinia Virus. Specific functions, and accordingly utilities, of VGAM255 correlate with, and may be deduced from, the identity of the host target genes which VGAM255 binds and inhibits, and the function of these host target genes, as elaborated hereinbelow.

Nucleotide sequences of the VGAM255 precursor RNA, herein designated VGAM PRECURSOR RNA, and of the ‘diced’ VGAM255 RNA, herein designated VGAM RNA, and a schematic representation of the secondary folding of VGAM255 folded precursor RNA, herein designated VGAM FOLDED PRECURSOR RNA, of VGAM255 are further described hereinbelow with reference to Table 1.

Nucleotide sequences of host target binding sites, such as BINDING SITE-I, BINDING SITE-II and BINDING SITE-III of FIG. 1, found on VGAM255 host target RNA, and schematic representation of the complementarity of each of these host target binding sites to VGAM255 RNA, herein designated VGAM RNA, are described hereinbelow with reference to Table 2.

As mentioned hereinabove with reference to FIG. 1, a function of VGAM255 gene, herein designated VGAM is inhibition of expression of VGAM255 target genes. It is appreciated that specific functions, and accordingly utilities, of VGAM255 correlate with, and may be deduced from, the identity of the target genes which VGAM255 binds and inhibits, and the function of these target genes, as elaborated hereinbelow.

Attractin (ATRN, Accession NM_{—}139321) is a VGAM255 host target gene. ATRN BINDING SITE is HOST TARGET binding site found in the 3′ untranslated region of mRNA encoded by ATRN, corresponding to a HOST TARGET binding site such as BINDING SITE I, BINDING SITE II or BINDING SITE III. Table 2 illustrates the complementarity of the nucleotide sequences of ATRN BINDING SITE, designated SEQ ID:2480, to the nucleotide sequence of VGAM255 RNA, herein designated VGAM RNA, also designated SEQ ID:590.

A function of VGAM255 is therefore inhibition of Attractin (ATRN, Accession NM_{—}139321), a gene which is involved in the initial immune cell clustering during inflammatory response. Accordingly, utilities of VGAM255 include diagnosis, prevention and treatment of diseases and clinical conditions associated with ATRN. The function of ATRN has been established by previous studies. Attractin is a human serum glycoprotein that is rapidly expressed on activated T cells and released extracellularly after 48 to 72 hours. Duke-Cohan et al. (1998) cloned attractin and found that, as in its natural serum form, it mediates the spreading of monocytes that becomes the focus for the clustering of nonproliferating T lymphocytes. There are 2 mRNA species with hematopoietic tissue-specific expression that code for a 134-kD protein with a putative serine protease catalytic serine, 4 EGF-like motifs, a CUB domain, a C-type lectin domain, and a domain homologous with the ligand-binding region of the common gamma cytokine chain. Except for the last 2 domains, the overall structure shares high homology with a protein of Caenorhabditis elegans, suggesting that attractin has evolved new domains and functions in parallel with the development of cell-mediated immunity. When attractin was identified as the product of the murine ‘mahogany’ gene with connections to control of pigmentation and energy metabolism, and the ‘mahogany’ product was identified and shown to be a transmembrane protein, the possibility of a human membrane attractin in addition to the secreted form was raised. Tang et al. (2000) described the complete genomic sequence of attractin, focusing in particular on the exons coding for the 3-prime region, and showed how both human membrane and secreted attractin arise as a result of alternate splicing of the same gene. They found that soluble attractin arises from transcription of 25 sequential exons on 20p13, where the 3-prime terminal exon contains sequence from a long interspersed nuclear element-1 (OMIM Ref. No. LINE-1) retrotransposon insertion that includes a stop codon and a polyadenylation signal. The mRNA isoform for membrane attraction splices over the LINE-1 exon and includes 5 exons encoding transmembrane and cytoplasmic domains with organization and coding potential almost identical to that of the mouse gene. The relative abundance of soluble and transmembrane isoforms measured by RT-PCR is differentially regulated in lymphoid tissues. Because activation of peripheral blood leukocytes with phytohemagglutinin induces strong expression of cell surface attractin followed by release of soluble attractin, these results suggested to Tang et al. (2000) that LINE-1 insertion, a genomic event unique to mammals, provided an evolutionarily mechanism for regulating cell interactions during an inflammatory reaction.

Full details of the abovementioned studies are described in the following publications, the disclosure of which are hereby incorporated by reference:
Duke-Cohan, J. S.; Gu, J.; McLaughlin, D. F.; Xu, Y.; Freeman, G. J.; Schlossman, S. F.: Attractin (DPPT-L), a member of the CUB family of cell adhesion and guidance proteins, is secreted by activated human T lymphocytes and modulates immune cell interactions. Proc. Nat. Acad. Sci. 95: 11336-11341, 1998.; and
Tang, W.; Gunn, T. M.; McLaughlin, D. F.; Barsh, G. S.; Schlossman, S. F.; Duke-Cohan, J. S.: Secreted and membrane attractin result from alternative splicing of the human ATRN gene. Pr.

Further studies establishing the function and utilities of ATRN are found in John Hopkins OMIM database record ID 603130, and in sited publications numbered 130-13 and 1186 listed in the bibliography section hereinbelow, which are also hereby incorporated by reference. CD59 Antigen P18-20 (antigen identified by monoclonal antibodies 16.3A5, EJ16, EJ30, EL32 and G344) (CD59, Accession NM_{—}000611) is another VGAM255 host target gene. CD59 BINDING SITE is HOST TARGET binding site found in the 3′ untranslated region of mRNA encoded by CD59, corresponding to a HOST TARGET binding site such as BINDING SITE I, BINDING SITE II or BINDING SITE III. Table 2 illustrates the complementarity of the nucleotide sequences of CD59 BINDING SITE, designated SEQ ID:760, to the nucleotide sequence of VGAM255 RNA, herein designated VGAM RNA, also designated SEQ ID:590.

Another function of VGAM255 is therefore inhibition of CD59 Antigen P18-20 (antigen identified by monoclonal antibodies 16.3A5, EJ16, EJ30, EL32 and G344) (CD59, Accession NM_{—}000611), a gene which restricts lysis of human erythrocytes and leukocytes by homologous complement. Accordingly, utilities of VGAM255 include diagnosis, prevention and treatment of diseases and clinical conditions associated with CD59. The function of CD59 has been established by previous studies. The CD59 antigen recognized by monoclonal antibody MEM-43 is an 18- to 25-kD glycoprotein expressed on all human peripheral blood leukocytes, erythrocytes, and several human cell lines. A close relationship to Ly6 of the mouse has been demonstrated. Antigens encoded by both Ly6 and CD59 genes are important to T-cell and NK-cell function. CD59 is also known as protectin. Its function is to restrict lysis of human erythrocytes and leukocytes by homologous complement. By directly incorporating protectin into membranes of heterologous cells, Meri et al. (1990) found that protectin does not prevent perforin-mediated killing (see OMIM Ref. No. 170280), whereas complement killing is effectively restricted. Thus, cell-mediated killing is unaffected by protectin. Meri et al. (1990) described the functional characteristics of protectin. Much attention has been focused on the Ly6 proteins because they may be involved in lymphocyte activation, and expression of some of them occurs at critical times in the differentiation of lymphocytes. Walsh et al. (1992) reviewed information on CD59, which they characterized as a multifunctional molecule with a role particularly in inhibition of formation of membrane attack complex. They raised the possibility that Ly6 is not a homolog and that the true MAC-inhibiting murine homolog of CD59 had yet to be found.

Full details of the abovementioned studies are described in the following publications, the disclosure of which are hereby incorporated by reference:
Meri, S.; Morgan, B. P.; Davies, A.; Daniels, R. H.; Olavesen, M. G.; Waldmann, H.; Lachmann, P. J.: Human protectin (CD59), an 18,000-20,000 MW complement lysis restricting factor, inhibits C5b-8 catalysed insertion of C9 into lipid bilayers. Immunology 71: 1-9, 1990.; and
Walsh, L. A.; Tone, M.; Thiru, S.; Waldmann, H.: The CD59 antigen—a multifunctional molecule. Tissue Antigens 40: 213-220, 1992.

Further studies establishing the function and utilities of CD59 are found in John Hopkins OMIM database record ID 107271, and in sited publications numbered 2906-2915, 683, 2916-2922, 2979-2985, 107 and 2986 listed in the bibliography section hereinbelow, which are also hereby incorporated by reference. Centaurin, Delta 1 (CENTD1, Accession NM_{—}015230) is another VGAM255 host target gene. CENTD1 BINDING SITE1 and CENTD1 BINDING SITE2 are HOST TARGET binding sites found in untranslated regions of mRNA encoded by CENTD1, corresponding to HOST TARGET binding sites such as BINDING SITE I, BINDING SITE II or BINDING SITE III. Table 2 illustrates the complementarity of the nucleotide sequences of CENTD1 BINDING SITE1 and CENTD1 BINDING SITE2, designated SEQ ID: 1615 and SEQ ID:2474 respectively, to the nucleotide sequence of VGAM255 RNA, herein designated VGAM RNA, also designated SEQ ID:590.

Another function of VGAM255 is therefore inhibition of Centaurin, Delta 1 (CENTD1, Accession NM_{—}015230), a gene which is involved in cell signaling/communication. Accordingly, utilities of VGAM255 include diagnosis, prevention and treatment of diseases and clinical conditions associated with CENTD1. The function of CENTD1 and its association with various diseases and clinical conditions, has been established by previous studies, as described hereinabove with reference to VGAM205. NADH Dehydrogenase (ubiquinone) 1 Alpha Subcomplex, 5, 13 kDa (NDUFA5, Accession NM_{—}005000) is another VGAM255 host target gene. NDUFA5 BINDING SITE is HOST TARGET binding site found in the 3′ untranslated region of mRNA encoded by NDUFA5, corresponding to a HOST TARGET binding site such as BINDING SITE I, BINDING SITE II or BINDING SITE III. Table 2 illustrates the complementarity of the nucleotide sequences of NDUFA5 BINDING SITE, designated SEQ ID:1169, to the nucleotide sequence of VGAM255 RNA, herein designated VGAM RNA, also designated SEQ ID:590.

Another function of VGAM255 is therefore inhibition of NADH Dehydrogenase (ubiquinone) 1 Alpha Subcomplex, 5, 13 kDa (NDUFA5, Accession NM_{—}005000), a gene which transfers electrons from nadh to the respiratory chain. Accordingly, utilities of VGAM255 include diagnosis, prevention and treatment of diseases and clinical conditions associated with NDUFA5. The function of NDUFA5 has been established by previous studies. The multisubunit NADH:ubiquinone oxidoreductase (complex I) is the first enzyme complex in the electron transport chain of mitochondria. The iron-sulfur protein (IP) fraction of complex I is made up of 7 subunits, including B13. See NDUFS1 (OMIM Ref. No. 157655). By a combination of EST database screening and PCR, Pata et al. (1997) isolated cDNAs encoding the human homolog of bovine B13. The deduced 116-amino acid human protein has a calculated molecular weight of approximately 13 kD. Human and bovine B13 are 87% identical on the amino acid level. Northern blot analysis revealed that the 1.6-kb B13 mRNA was expressed in all human tissues tested, with the highest levels in heart, skeletal muscle, and brain. Two additional smaller transcripts were also detected. Using Southern blots, Pata et al. (1997) determined that B13 is part of a multigene family in humans. During the course of a physical mapping project on 11p15.5, Russell et al. (1997) identified sequence with a high degree of similarity to the bovine NADH:ubiquinone oxidoreductase subunit B13. Following up on this lead, they isolated a clone with nucleotide sequence 88% and 83% identical over the predicted open reading frame with bovine and rat B13 subunit genes, respectively. The position of the initiation and termination codons was conserved. To determine the chromosomal localization of the B13 subunit gene, they screened a monochromosome somatic cell hybrid panel and showed that only the hybrid containing human chromosome 7 was positive.

Full details of the abovementioned studies are described in the following publications, the disclosure of which are hereby incorporated by reference:
Pata, I.; Tensing, K.; Metspalu, A.: A human cDNA encoding the homologue of NADH:ubiquinone oxidoreductase subunit B13. Biochim. Biophys. Acta 1350: 115-118, 1997.; and
Russell, M. W.; du Manoir, S.; Collins, F. S.; Brody, L. C.: Cloning of the human NADH:ubiquinone oxidoreductase subunit B13: localization to chromosome 7q32 and identification of a ps.

Further studies establishing the function and utilities of NDUFA5 are found in John Hopkins OMIM database record ID 601677, and in sited publications numbered 1442-1443 listed in the bibliography section hereinbelow, which are also hereby incorporated by reference. Pre-B-cell Leukemia Transcription Factor 3 (PBX3, Accession NM_{—}006195) is another VGAM255 host target gene. PBX3 BINDING SITE is HOST TARGET binding site found in the 3′ untranslated region of mRNA encoded by PBX3, corresponding to a HOST TARGET binding site such as BINDING SITE I, BINDING SITE II or BINDING SITE III. Table 2 illustrates the complementarity of the nucleotide sequences of PBX3 BINDING SITE, designated SEQ ID:1279, to the nucleotide sequence of VGAM255 RNA, herein designated VGAM RNA, also designated SEQ ID:590.

Another function of VGAM255 is therefore inhibition of Pre-B-cell Leukemia Transcription Factor 3 (PBX3, Accession NM_{—}006195). Accordingly, utilities of VGAM255 include diagnosis, prevention and treatment of diseases and clinical conditions associated with PBX3. Stearoyl-CoA Desaturase (delta-9-desaturase) (SCD, Accession NM_{—}005063) is another VGAM255 host target gene. SCD BINDING SITE is HOST TARGET binding site found in the 3′ untranslated region of mRNA encoded by SCD, corresponding to a HOST TARGET binding site such as BINDING SITE I, BINDING SITE II or BINDING SITE III. Table 2 illustrates the complementarity of the nucleotide sequences of SCD BINDING SITE, designated SEQ ID:1178, to the nucleotide sequence of VGAM255 RNA, herein designated VGAM RNA, also designated SEQ ID:590.

Another function of VGAM255 is therefore inhibition of Stearoyl-CoA Desaturase (delta-9-desaturase) (SCD, Accession NM_{—}005063), a gene which functions in the synthesis of unsaturated fatty acids. Accordingly, utilities of VGAM255 include diagnosis, prevention and treatment of diseases and clinical conditions associated with SCD. The function of SCD and its association with various diseases and clinical conditions, has been established by previous studies, as described hereinabove with reference to VGAM155. Solute Carrier Family 2 (facilitated glucose transporter), Member 2 (SLC2A2, Accession NM_{—}000340) is another VGAM255 host target gene. SLC2A2 BINDING SITE is HOST TARGET binding site found in the 3′ untranslated region of mRNA encoded by SLC2A2, corresponding to a HOST TARGET binding site such as BINDING SITE I, BINDING SITE II or BINDING SITE III. Table 2 illustrates the complementarity of the nucleotide sequences of SLC2A2 BINDING SITE, designated SEQ ID:731, to the nucleotide sequence of VGAM255 RNA, herein designated VGAM RNA, also designated SEQ ID:590.

Another function of VGAM255 is therefore inhibition of Solute Carrier Family 2 (facilitated glucose transporter), Member 2 (SLC2A2, Accession NM_{—}000340). Accordingly, utilities of VGAM255 include diagnosis, prevention and treatment of diseases and clinical conditions associated with SLC2A2. Transcription Factor 7 (T-cell specific, HMG-box) (TCF7, Accession NM_{—}003202) is another VGAM255 host target gene. TCF7 BINDING SITE is HOST TARGET binding site found in the 3′ untranslated region of mRNA encoded by TCF7, corresponding to a HOST TARGET binding site such as BINDING SITE I, BINDING SITE II or BINDING SITE III. Table 2 illustrates the complementarity of the nucleotide sequences of TCF7 BINDING SITE, designated SEQ ID:998, to the nucleotide sequence of VGAM255 RNA, herein designated VGAM RNA, also designated SEQ ID:590.

Another function of VGAM255 is therefore inhibition of Transcription Factor 7 (T-cell specific, HMG-box) (TCF7, Accession NM_{—}003202). Accordingly, utilities of VGAM255 include diagnosis, prevention and treatment of diseases and clinical conditions associated with TCF7. DKFZP586B0923 (Accession XM_{—}167606) is another VGAM255 host target gene. DKFZP586B0923 BINDING SITE is HOST TARGET binding site found in the 3′ untranslated region of mRNA encoded by DKFZP586B0923, corresponding to a HOST TARGET binding site such as BINDING SITE I, BINDING SITE II or BINDING SITE III. Table 2 illustrates the complementarity of the nucleotide sequences of DKFZP586B0923 BINDING SITE, designated SEQ ID:3589, to the nucleotide sequence of VGAM255 RNA, herein designated VGAM RNA, also designated SEQ ID:590.

Another function of VGAM255 is therefore inhibition of DKFZP586B0923 (Accession XM_{—}167606). Accordingly, utilities of VGAM255 include diagnosis, prevention and treatment of diseases and clinical conditions associated with DKFZP586B0923. DKFZp761K1824 (Accession NM_{—}017597) is another VGAM255 host target gene. DKFZp761K1824 BINDING SITE is HOST TARGET binding site found in the 3′ untranslated region of mRNA encoded by DKFZp761K1824, corresponding to a HOST TARGET binding site such as BINDING SITE I, BINDING SITE II or BINDING SITE III. Table 2 illustrates the complementarity of the nucleotide sequences of DKFZp761K1824 BINDING SITE, designated SEQ ID:1726, to the nucleotide sequence of VGAM255 RNA, herein designated VGAM RNA, also designated SEQ ID:590.

Another function of VGAM255 is therefore inhibition of DKFZp761K1824 (Accession NM_{—}017597). Accordingly, utilities of VGAM255 include diagnosis, prevention and treatment of diseases and clinical conditions associated with DKFZp761K1824. FLJ14624 (Accession XM_{—}049060) is another VGAM255 host target gene. FLJ14624 BINDING SITE is HOST TARGET binding site found in the 3′ untranslated region of mRNA encoded by FLJ14624, corresponding to a HOST TARGET binding site such as BINDING SITE I, BINDING SITE II or BINDING SITE III. Table 2 illustrates the complementarity of the nucleotide sequences of FLJ14624 BINDING SITE, designated SEQ ID:2913, to the nucleotide sequence of VGAM255 RNA, herein designated VGAM RNA, also designated SEQ ID:590.

Another function of VGAM255 is therefore inhibition of FLJ14624 (Accession XM_{—}049060). Accordingly, utilities of VGAM255 include diagnosis, prevention and treatment of diseases and clinical conditions associated with FLJ14624. GRO3 (Accession XM_{—}031287) is another VGAM255 host target gene. GRO3 BINDING SITE is HOST TARGET binding site found in the 3′ untranslated region of mRNA encoded by GRO3, corresponding to a HOST TARGET binding site such as BINDING SITE I, BINDING SITE II or BINDING SITE III. Table 2 illustrates the complementarity of the nucleotide sequences of GRO3 BINDING SITE, designated SEQ ID:2636, to the nucleotide sequence of VGAM255 RNA, herein designated VGAM RNA, also designated SEQ ID:590.

Another function of VGAM255 is therefore inhibition of GRO3 (Accession XM_{—}031287). Accordingly, utilities of VGAM255 include diagnosis, prevention and treatment of diseases and clinical conditions associated with GRO3. KIAA0907 (Accession NM_{—}014949) is another VGAM255 host target gene. KIAA0907 BINDING SITE is HOST TARGET binding site found in the 3′ untranslated region of mRNA encoded by KIAA0907, corresponding to a HOST TARGET binding site such as BINDING SITE I, BINDING SITE II or BINDING SITE III. Table 2 illustrates the complementarity of the nucleotide sequences of KIAA0907 BINDING SITE, designated SEQ ID:1596, to the nucleotide sequence of VGAM255 RNA, herein designated VGAM RNA, also designated SEQ ID:590.

Another function of VGAM255 is therefore inhibition of KIAA0907 (Accession NM_{—}014949). Accordingly, utilities of VGAM255 include diagnosis, prevention and treatment of diseases and clinical conditions associated with KIAA0907. MGC14289 (Accession NM_{—}080660) is another VGAM255 host target gene. MGC14289 BINDING SITE is HOST TARGET binding site found in the 3′ untranslated region of mRNA encoded by MGC14289, corresponding to a HOST TARGET binding site such as BINDING SITE I, BINDING SITE II or BINDING SITE III. Table 2 illustrates the complementarity of the nucleotide sequences of MGC14289 BINDING SITE, designated SEQ ID:2382, to the nucleotide sequence of VGAM255 RNA, herein designated VGAM RNA, also designated SEQ ID:590.

Another function of VGAM255 is therefore inhibition of MGC14289 (Accession NM_{—}080660). Accordingly, utilities of VGAM255 include diagnosis, prevention and treatment of diseases and clinical conditions associated with MGC14289. NLP_{—}1 (Accession NM_{—}007342) is another VGAM255 host target gene. NLP_{—}1 BINDING SITE is HOST TARGET binding site found in the 3′ untranslated region of mRNA encoded by NLP_{—}1, corresponding to a HOST TARGET binding site such as BINDING SITE I, BINDING SITE II or BINDING SITE III. Table 2 illustrates the complementarity of the nucleotide sequences of NLP_{—}1 BINDING SITE, designated SEQ ID:1395, to the nucleotide sequence of VGAM255 RNA, herein designated VGAM RNA, also designated SEQ ID:590.

Another function of VGAM255 is therefore inhibition of NLP_{—}1 (Accession NM_{—}007342). Accordingly, utilities of VGAM255 include diagnosis, prevention and treatment of diseases and clinical conditions associated with NLP_{—}1. Parvin, Alpha (PARVA, Accession NM_{—}018222) is another VGAM255 host target gene. PARVA BINDING SITE is HOST TARGET binding site found in the 3′ untranslated region of mRNA encoded by PARVA, corresponding to a HOST TARGET binding site such as BINDING SITE I, BINDING SITE II or BINDING SITE III. Table 2 illustrates the complementarity of the nucleotide sequences of PARVA BINDING SITE, designated SEQ ID:1802, to the nucleotide sequence of VGAM255 RNA, herein designated VGAM RNA, also designated SEQ ID:590.

Another function of VGAM255 is therefore inhibition of Parvin, Alpha (PARVA, Accession NM_{—}018222). Accordingly, utilities of VGAM255 include diagnosis, prevention and treatment of diseases and clinical conditions associated with PARVA. PRO2176 (Accession NM_{—}018515) is another VGAM255 host target gene. PRO2176 BINDING SITE is HOST TARGET binding site found in the 3′ untranslated region of mRNA encoded by PRO2176, corresponding to a HOST TARGET binding site such as BINDING SITE I, BINDING SITE II or BINDING SITE III. Table 2 illustrates the complementarity of the nucleotide sequences of PRO2176 BINDING SITE, designated SEQ ID:1833, to the nucleotide sequence of VGAM255 RNA, herein designated VGAM RNA, also designated SEQ ID:590.

Another function of VGAM255 is therefore inhibition of PRO2176 (Accession NM_{—}018515). Accordingly, utilities of VGAM255 include diagnosis, prevention and treatment of diseases and clinical conditions associated with PRO2176. Translocase of Inner Mitochondrial Membrane 22 Homolog (yeast) (TIMM22, Accession XM_{—}085739) is another VGAM255 host target gene. TIMM22 BINDING SITE is HOST TARGET binding site found in the 3′ untranslated region of mRNA encoded by TIMM22, corresponding to a HOST TARGET binding site such as BINDING SITE I, BINDING SITE II or BINDING SITE III. Table 2 illustrates the complementarity of the nucleotide sequences of TIMM22 BINDING SITE, designated SEQ ID:3104, to the nucleotide sequence of VGAM255 RNA, herein designated VGAM RNA, also designated SEQ ID:590.

Another function of VGAM255 is therefore inhibition of Translocase of Inner Mitochondrial Membrane 22 Homolog (yeast) (TIMM22, Accession XM_{—}085739). Accordingly, utilities of VGAM255 include diagnosis, prevention and treatment of diseases and clinical conditions associated with TIMM22. LOC143914 (Accession XM_{—}084654) is another VGAM255 host target gene. LOC143914 BINDING SITE is HOST TARGET binding site found in the 5′ untranslated region of mRNA encoded by LOC143914, corresponding to a HOST TARGET binding site such as BINDING SITE I, BINDING SITE II or BINDING SITE III. Table 2 illustrates the complementarity of the nucleotide sequences of LOC143914 BINDING SITE, designated SEQ ID:3061, to the nucleotide sequence of VGAM255 RNA, herein designated VGAM RNA, also designated SEQ ID:590.

Another function of VGAM255 is therefore inhibition of LOC143914 (Accession XM_{—}084654). Accordingly, utilities of VGAM255 include diagnosis, prevention and treatment of diseases and clinical conditions associated with LOC143914. LOC154862 (Accession XM_{—}088061) is another VGAM255 host target gene. LOC154862 BINDING SITE is HOST TARGET binding site found in the 3′ untranslated region of mRNA encoded by LOC154862, corresponding to a HOST TARGET binding site such as BINDING SITE I, BINDING SITE II or BINDING SITE III. Table 2 illustrates the complementarity of the nucleotide sequences of LOC154862 BINDING SITE, designated SEQ ID:3193, to the nucleotide sequence of VGAM255 RNA, herein designated VGAM RNA, also designated SEQ ID:590.

Another function of VGAM255 is therefore inhibition of LOC154862 (Accession XM_{—}088061). Accordingly, utilities of VGAM255 include diagnosis, prevention and treatment of diseases and clinical conditions associated with LOC154862. LOC158314 (Accession XM_{—}098920) is another VGAM255 host target gene. LOC158314 BINDING SITE is HOST TARGET binding site found in the 3′ untranslated region of mRNA encoded by LOC158314, corresponding to a HOST TARGET binding site such as BINDING SITE I, BINDING SITE II or BINDING SITE III. Table 2 illustrates the complementarity of the nucleotide sequences of LOC158314 BINDING SITE, designated SEQ ID:3386, to the nucleotide sequence of VGAM255 RNA, herein designated VGAM RNA, also designated SEQ ID:590.

Another function of VGAM255 is therefore inhibition of LOC158314 (Accession XM_{—}098920). Accordingly, utilities of VGAM255 include diagnosis, prevention and treatment of diseases and clinical conditions associated with LOC158314. LOC221405 (Accession XM_{—}168138) is another VGAM255 host target gene. LOC221405 BINDING SITE is HOST TARGET binding site found in the 3′ untranslated region of mRNA encoded by LOC221405, corresponding to a HOST TARGET binding site such as BINDING SITE I, BINDING SITE II or BINDING SITE III. Table 2 illustrates the complementarity of the nucleotide sequences of LOC221405 BINDING SITE, designated SEQ ID:3623, to the nucleotide sequence of VGAM255 RNA, herein designated VGAM RNA, also designated SEQ ID:590.

Another function of VGAM255 is therefore inhibition of LOC221405 (Accession XM_{—}168138). Accordingly, utilities of VGAM255 include diagnosis, prevention and treatment of diseases and clinical conditions associated with LOC221405. FIG. 1 further provides a conceptual description of a novel bioinformatically detected viral gene of the present invention, referred to here as Viral Genomic Address Messenger 256 (VGAM256) viral gene, which modulates expression of respective host target genes thereof, the function and utility of which host target genes is known in the art.

VGAM256 is a novel bioinformatically detected regulatory, non protein coding, viral micro RNA (miRNA) gene. The method by which VGAM256 was detected is described hereinabove with reference to FIGS. 1-8.

VGAM256 gene, herein designated VGAM GENE, is a viral gene contained in the genome of Vaccinia Virus. VGAM256 host target gene, herein designated VGAM HOST TARGET GENE, is a human gene contained in the human genome.

VGAM256 gene encodes a VGAM256 precursor RNA, herein designated VGAM PRECURSOR RNA. Similar to other miRNA genes, and unlike most ordinary genes, VGAM256 precursor RNA does not encode a protein. A nucleotide sequence identical or highly similar to the nucleotide sequence of VGAM256 precursor RNA is designated SEQ ID:242, and is provided hereinbelow with reference to the sequence listing part. Nucleotide sequence SEQ ID:242 is located at position 143051 relative to the genome of Vaccinia Virus.

VGAM256 precursor RNA folds onto itself, forming VGAM256 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM256 folded precursor RNA into VGAM256 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM256 RNA is designated SEQ ID:591, and is provided hereinbelow with reference to the sequence listing part.

VGAM256 host target gene, herein designated VGAM HOST TARGET GENE, encodes a corresponding messenger RNA, VGAM256 host target RNA, herein designated VGAM HOST TARGET RNA. VGAM256 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM256 RNA, herein designated VGAM RNA, binds complementarily to one or more host target binding sites located in untranslated regions of VGAM256 host target RNA, herein designated VGAM HOST TARGET RNA. This complementary binding is due to the fact that the nucleotide sequence of VGAM256 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM256 RNA, herein designated VGAM RNA, may have a different number of host target binding sites in untranslated regions of a VGAM256 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM256 RNA, herein designated VGAM RNA, to host target binding sites on VGAM256 host target RNA, herein designated VGAM HOST TARGET RNA, such as BINDING SITE I, BINDING SITE II and BINDING SITE III, inhibits translation of VGAM256 host target RNA into VGAM256 host target protein, herein designated VGAM HOST TARGET PROTEIN. VGAM host target protein is therefore outlined by a broken line.

It is appreciated that VGAM256 host target gene, herein designated VGAM HOST TARGET GENE, in fact represents a plurality of VGAM256 host target genes. The mRNA of each one of this plurality of VGAM256 host target genes comprises one or more host target binding sites, each having a nucleotide sequence which is at least partly complementary to VGAM256 RNA, herein designated VGAM RNA, and which when bound by VGAM256 RNA causes inhibition of translation of respective one or more VGAM256 host target proteins.

It is further appreciated by one skilled in the art that the mode of translational inhibition illustrated by FIG. 1 with specific reference to translational inhibition exerted by VGAM256 gene, herein designated VGAM GENE, on one or more VGAM256 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM256 is inhibition of expression of host target genes, as part of a novel viral mechanism of attacking a host. Accordingly, utilities of VGAM256 include diagnosis, prevention and treatment of viral infection by Vaccinia Virus. Specific functions, and accordingly utilities, of VGAM256 correlate with, and may be deduced from, the identity of the host target genes which VGAM256 binds and inhibits, and the function of these host target genes, as elaborated hereinbelow.

Nucleotide sequences of the VGAM256 precursor RNA, herein designated VGAM PRECURSOR RNA, and of the ‘diced’ VGAM256 RNA, herein designated VGAM RNA, and a schematic representation of the secondary folding of VGAM256 folded precursor RNA, herein designated VGAM FOLDED PRECURSOR RNA, of VGAM256 are further described hereinbelow with reference to Table 1.

Nucleotide sequences of host target binding sites, such as BINDING SITE-I, BINDING SITE-II and BINDING SITE-III of FIG. 1, found on VGAM256 host target RNA, and schematic representation of the complementarity of each of these host target binding sites to VGAM256 RNA, herein designated VGAM RNA, are described hereinbelow with reference to Table 2.

As mentioned hereinabove with reference to FIG. 1, a function of VGAM256 gene, herein designated VGAM is inhibition of expression of VGAM256 target genes. It is appreciated that specific functions, and accordingly utilities, of VGAM256 correlate with, and may be deduced from, the identity of the target genes which VGAM256 binds and inhibits, and the function of these target genes, as elaborated hereinbelow.

V-yes-1 Yamaguchi Sarcoma Viral Oncogene Homolog 1 (YES1, Accession NM_{—}005433) is a VGAM256 host target gene. YES1 BINDING SITE is HOST TARGET binding site found in the 3′ untranslated region of mRNA encoded by YES1, corresponding to a HOST TARGET binding site such as BINDING SITE I, BINDING SITE II or BINDING SITE III. Table 2 illustrates the complementarity of the nucleotide sequences of YES1 BINDING SITE, designated SEQ ID:1209, to the nucleotide sequence of VGAM256 RNA, herein designated VGAM RNA, also designated SEQ ID:591.

A function of VGAM256 is therefore inhibition of V-yes-1 Yamaguchi Sarcoma Viral Oncogene Homolog 1 (YES1, Accession NM_{—}005433), a gene which is a putative protein-tyrosine kinase. Accordingly, utilities of VGAM256 include diagnosis, prevention and treatment of diseases and clinical conditions associated with YES1. The function of YES1 has been established by previous studies. The YES oncogene is homologous to the gene of the Yamaguchi sarcoma virus. The product of the gene is associated with tyrosine-specific protein kinase activity and its amino acid sequence shows a high degree of homology with that of the SRC gene product of Rous sarcoma virus. Semba et al. (1985) found in DNA from human embryo fibroblasts 10 EcoRI fragments that hybridized with the Yamaguchi sarcoma virus oncogene. Four of these (designated YES1) were assigned to chromosome 18 and 1 (designated YES2) was assigned to chromosome 6 by a study of human-mouse cell hybrids. (YES2 was later found (Semba et al., 1988) to be a pseudogene of YES1 and to be located at 22q11.2. Semba et al. (1988) stated: ‘The failure of proper mapping in our earlier experiment might have been caused by instability of hybrid cell clones.’) The other 5 fragments could not be mapped either because hybridization signals were too weak or differentiation from mouse YES fragments was impossible. There was evidence for multiple copies of YES-related genes in the human genome, but only a single RNA species, 4.8 kb long, was found. At least 3 of the human YES gene copies had both introns and exons and 1 gene copy appeared to be a pseudogene. By isotopic in situ hybridization, Yoshida et al. (1985) mapped the YES1 gene to 18q21.3. These workers suggested that the localization is consistent with a role in the pathogenesis of follicular lymphoma, which is frequently associated with a 14;18 translocation with the breakpoint at 18q21 (Fukuhara et al., 1979); see 151430. Ohno et al. (1987) found that although it is in the same chromosome region as BCL2 (OMIM Ref. No. 151430), the YES gene is intact in cases of follicular lymphoma. Using yeast artificial chromosomes (YACs) containing the YES1 gene as probes and fluorescence in situ hybridization, Silverman et al. (1993) detected a strong signal in the region corresponding to 18p11.3. These YACs were found to contain another 18p11.32 gene, thymidylate synthase (OMIM Ref. No. 188350); the genes were less than 50 kb apart. Overhauser et al. (1993) identified a sequence tagged site (STS) in the YES1 gene and used it in studies of somatic cell hybrids with deletion of various segments of chromosome 18 to map the gene to 18pter-p11.21.

Full details of the abovementioned studies are described in the following publications, the disclosure of which are hereby incorporated by reference:
Semba, K.; Yamanashi, Y.; Nishizawa, M.; Sukegawa, J.; Yoshida, M.; Sasaki, M.; Yamamoto, T.; Toyoshima, K.: Location of the c-yes gene on the human chromosome and its expression in various tissues. Science 227: 1038-1040, 1985.; and
Overhauser, J.; Mewar, R.; Rojas, K.; Lia, K.; Kline, A. D.; Silverman, G. A.: STS map of genes and anonymous DNA fragments on human chromosome 18 using a panel of somatic cell hybrids.

Further studies establishing the function and utilities of YES1 are found in John Hopkins OMIM database record ID 164880, and in sited publications numbered 703-704, 2762-70 and 360 listed in the bibliography section hereinbelow, which are also hereby incorporated by reference. ARP3 Actin-related Protein 3 Homolog (yeast) (ACTR3, Accession NM_{—}005721) is another VGAM256 host target gene. ACTR3 BINDING SITE is HOST TARGET binding site found in the 3′ untranslated region of mRNA encoded by ACTR3, corresponding to a HOST TARGET binding site such as BINDING SITE I, BINDING SITE II or BINDING SITE III. Table 2 illustrates the complementarity of the nucleotide sequences of ACTR3 BINDING SITE, designated SEQ ID:1238, to the nucleotide sequence of VGAM256 RNA, herein designated VGAM RNA, also designated SEQ ID:591.

Another function of VGAM256 is therefore inhibition of ARP3 Actin-related Protein 3 Homolog (yeast) (ACTR3, Accession NM_{—}005721). Accordingly, utilities of VGAM256 include diagnosis, prevention and treatment of diseases and clinical conditions associated with ACTR3. GENX-3414 (Accession NM_{—}003943) is another VGAM256 host target gene. GENX-3414 BINDING SITE is HOST TARGET binding site found in the 3′ untranslated region of mRNA encoded by GENX-3414, corresponding to a HOST TARGET binding site such as BINDING SITE I, BINDING SITE II or BINDING SITE III. Table 2 illustrates the complementarity of the nucleotide sequences of GENX-3414 BINDING SITE, designated SEQ ID: 1073, to the nucleotide sequence of VGAM256 RNA, herein designated VGAM RNA, also designated SEQ ID:591.

Another function of VGAM256 is therefore inhibition of GENX-3414 (Accession NM_{—}003943). Accordingly, utilities of VGAM256 include diagnosis, prevention and treatment of diseases and clinical conditions associated with GENX-3414. HT007 (Accession NM_{—}018480) is another VGAM256 host target gene. HT007 BINDING SITE is HOST TARGET binding site found in the 3′ untranslated region of mRNA encoded by HT007, corresponding to a HOST TARGET binding site such as BINDING SITE I, BINDING SITE II or BINDING SITE III. Table 2 illustrates the complementarity of the nucleotide sequences of HT007 BINDING SITE, designated SEQ ID:1832, to the nucleotide sequence of VGAM256 RNA, herein designated VGAM RNA, also designated SEQ ID:591.

Another function of VGAM256 is therefore inhibition of HT007 (Accession NM_{—}018480). Accordingly, utilities of VGAM256 include diagnosis, prevention and treatment of diseases and clinical conditions associated with HT007. Sprouty Homolog 4 (Drosophila) (SPRY4, Accession NM_{—}030964) is another VGAM256 host target gene. SPRY4 BINDING SITE is HOST TARGET binding site found in the 3′ untranslated region of mRNA encoded by SPRY4, corresponding to a HOST TARGET binding site such as BINDING SITE I, BINDING SITE II or BINDING SITE III. Table 2 illustrates the complementarity of the nucleotide sequences of SPRY4 BINDING SITE, designated SEQ ID:2175, to the nucleotide sequence of VGAM256 RNA, herein designated VGAM RNA, also designated SEQ ID:591.

Another function of VGAM256 is therefore inhibition of Sprouty Homolog 4 (Drosophila) (SPRY4, Accession NM_{—}030964). Accordingly, utilities of VGAM256 include diagnosis, prevention and treatment of diseases and clinical conditions associated with SPRY4. FIG. 1 further provides a conceptual description of a novel bioinformatically detected viral gene of the present invention, referred to here as Viral Genomic Address Messenger 257 (VGAM257) viral gene, which modulates expression of respective host target genes thereof, the function and utility of which host target genes is known in the art.

VGAM257 is a novel bioinformatically detected regulatory, non protein coding, viral micro RNA (miRNA) gene. The method by which VGAM257 was detected is described hereinabove with reference to FIGS. 1-8.

VGAM257 gene, herein designated VGAM GENE, is a viral gene contained in the genome of Vaccinia Virus. VGAM257 host target gene, herein designated VGAM HOST TARGET GENE, is a human gene contained in the human genome.

VGAM257 gene encodes a VGAM257 precursor RNA, herein designated VGAM PRECURSOR RNA. Similar to other miRNA genes, and unlike most ordinary genes, VGAM257 precursor RNA does not encode a protein. A nucleotide sequence identical or highly similar to the nucleotide sequence of VGAM257 precursor RNA is designated SEQ ID:243, and is provided hereinbelow with reference to the sequence listing part. Nucleotide sequence SEQ ID:243 is located at position 142696 relative to the genome of Vaccinia Virus.

VGAM257 precursor RNA folds onto itself, forming VGAM257 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM257 folded precursor RNA into VGAM257 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM257 RNA is designated SEQ ID:592, and is provided hereinbelow with reference to the sequence listing part.

VGAM257 host target gene, herein designated VGAM HOST TARGET GENE, encodes a corresponding messenger RNA, VGAM257 host target RNA, herein designated VGAM HOST TARGET RNA. VGAM257 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM257 RNA, herein designated VGAM RNA, binds complementarily to one or more host target binding sites located in untranslated regions of VGAM257 host target RNA, herein designated VGAM HOST TARGET RNA. This complementary binding is due to the fact that the nucleotide sequence of VGAM257 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM257 RNA, herein designated VGAM RNA, may have a different number of host target binding sites in untranslated regions of a VGAM257 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM257 RNA, herein designated VGAM RNA, to host target binding sites on VGAM257 host target RNA, herein designated VGAM HOST TARGET RNA, such as BINDING SITE I, BINDING SITE II and BINDING SITE III, inhibits translation of VGAM257 host target RNA into VGAM257 host target protein, herein designated VGAM HOST TARGET PROTEIN. VGAM host target protein is therefore outlined by a broken line.

It is appreciated that VGAM257 host target gene, herein designated VGAM HOST TARGET GENE, in fact represents a plurality of VGAM257 host target genes. The mRNA of each one of this plurality of VGAM257 host target genes comprises one or more host target binding sites, each having a nucleotide sequence which is at least partly complementary to VGAM257 RNA, herein designated VGAM RNA, and which when bound by VGAM257 RNA causes inhibition of translation of respective one or more VGAM257 host target proteins.

It is further appreciated by one skilled in the art that the mode of translational inhibition illustrated by FIG. 1 with specific reference to translational inhibition exerted by VGAM257 gene, herein designated VGAM GENE, on one or more VGAM257 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM257 is inhibition of expression of host target genes, as part of a novel viral mechanism of attacking a host. Accordingly, utilities of VGAM257 include diagnosis, prevention and treatment of viral infection by Vaccinia Virus. Specific functions, and accordingly utilities, of VGAM257 correlate with, and may be deduced from, the identity of the host target genes which VGAM257 binds and inhibits, and the function of these host target genes, as elaborated hereinbelow.

Nucleotide sequences of the VGAM257 precursor RNA, herein designated VGAM PRECURSOR RNA, and of the ‘diced’ VGAM257 RNA, herein designated VGAM RNA, and a schematic representation of the secondary folding of VGAM257 folded precursor RNA, herein designated VGAM FOLDED PRECURSOR RNA, of VGAM257 are further described hereinbelow with reference to Table 1.

Nucleotide sequences of host target binding sites, such as BINDING SITE-I, BINDING SITE-II and BINDING SITE-III of FIG. 1, found on VGAM257 host target RNA, and schematic representation of the complementarity of each of these host target binding sites to VGAM257 RNA, herein designated VGAM RNA, are described hereinbelow with reference to Table 2.

As mentioned hereinabove with reference to FIG. 1, a function of VGAM257 gene, herein designated VGAM is inhibition of expression of VGAM257 target genes. It is appreciated that specific functions, and accordingly utilities, of VGAM257 correlate with, and may be deduced from, the identity of the target genes which VGAM257 binds and inhibits, and the function of these target genes, as elaborated hereinbelow.

POF1B (Accession NM_{—}024921) is a VGAM257 host target gene. POF1B BINDING SITE is HOST TARGET binding site found in the 3′ untranslated region of mRNA encoded by POF1B, corresponding to a HOST TARGET binding site such as BINDING SITE I, BINDING SITE II or BINDING SITE III. Table 2 illustrates the complementarity of the nucleotide sequences of POF1B BINDING SITE, designated SEQ ID:2114, to the nucleotide sequence of VGAM257 RNA, herein designated VGAM RNA, also designated SEQ ID:592.

A function of VGAM257 is therefore inhibition of POF1B (Accession NM_{—}024921). Accordingly, utilities of VGAM257 include diagnosis, prevention and treatment of diseases and clinical conditions associated with POF1B. RODH-4 (Accession NM_{—}003708) is another VGAM257 host target gene. RODH-4 BINDING SITE is HOST TARGET binding site found in the 5′ untranslated region of mRNA encoded by RODH-4, corresponding to a HOST TARGET binding site such as BINDING SITE I, BINDING SITE II or BINDING SITE III. Table 2 illustrates the complementarity of the nucleotide sequences of RODH-4 BINDING SITE, designated SEQ ID: 1050, to the nucleotide sequence of VGAM257 RNA, herein designated VGAM RNA, also designated SEQ ID:592.

Another function of VGAM257 is therefore inhibition of RODH-4 (Accession NM_{—}003708). Accordingly, utilities of VGAM257 include diagnosis, prevention and treatment of diseases and clinical conditions associated with RODH-4. Splicing Factor, Arginine/serine-rich 12 (SFRS12, Accession NM_{—}139168) is another VGAM257 host target gene. SFRS12 BINDING SITE is HOST TARGET binding site found in the 3′ untranslated region of mRNA encoded by SFRS12, corresponding to a HOST TARGET binding site such as BINDING SITE I, BINDING SITE II or BINDING SITE III. Table 2 illustrates the complementarity of the nucleotide sequences of SFRS12 BINDING SITE, designated SEQ ID:2472, to the nucleotide sequence of VGAM257 RNA, herein designated VGAM RNA, also designated SEQ ID:592.

Another function of VGAM257 is therefore inhibition of Splicing Factor, Arginine/serine-rich 12 (SFRS12, Accession NM_{—}139168). Accordingly, utilities of VGAM257 include diagnosis, prevention and treatment of diseases and clinical conditions associated with SFRS12. TCBAP0758 (Accession NM_{—}030973) is another VGAM257 host target gene. TCBAP0758 BINDING SITE is HOST TARGET binding site found in the 3′ untranslated region of mRNA encoded by TCBAP0758, corresponding to a HOST TARGET binding site such as BINDING SITE I, BINDING SITE II or BINDING SITE III. Table 2 illustrates the complementarity of the nucleotide sequences of TCBAP0758 BINDING SITE, designated SEQ ID:2181, to the nucleotide sequence of VGAM257 RNA, herein designated VGAM RNA, also designated SEQ ID:592.

Another function of VGAM257 is therefore inhibition of TCBAP0758 (Accession NM_{—}030973). Accordingly, utilities of VGAM257 include diagnosis, prevention and treatment of diseases and clinical conditions associated with TCBAP0758. FIG. 1 further provides a conceptual description of a novel bioinformatically detected viral gene of the present invention, referred to here as Viral Genomic Address Messenger 258 (VGAM258) viral gene, which modulates expression of respective host target genes thereof, the function and utility of which host target genes is known in the art.

VGAM258 is a novel bioinformatically detected regulatory, non protein coding, viral micro RNA (miRNA) gene. The method by which VGAM258 was detected is described hereinabove with reference to FIGS. 1-8.

VGAM258 gene, herein designated VGAM GENE, is a viral gene contained in the genome of Vaccinia Virus. VGAM258 host target gene, herein designated VGAM HOST TARGET GENE, is a human gene contained in the human genome.

VGAM258 gene encodes a VGAM258 precursor RNA, herein designated VGAM PRECURSOR RNA. Similar to other miRNA genes, and unlike most ordinary genes, VGAM258 precursor RNA does not encode a protein. A nucleotide sequence identical or highly similar to the nucleotide sequence of VGAM258 precursor RNA is designated SEQ ID:244, and is provided hereinbelow with reference to the sequence listing part. Nucleotide sequence SEQ ID:244 is located at position 141997 relative to the genome of Vaccinia Virus.

VGAM258 precursor RNA folds onto itself, forming VGAM258 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM258 folded precursor RNA into VGAM258 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM258 RNA is designated SEQ ID:593, and is provided hereinbelow with reference to the sequence listing part.

VGAM258 host target gene, herein designated VGAM HOST TARGET GENE, encodes a corresponding messenger RNA, VGAM258 host target RNA, herein designated VGAM HOST TARGET RNA. VGAM258 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM258 RNA, herein designated VGAM RNA, binds complementarily to one or more host target binding sites located in untranslated regions of VGAM258 host target RNA, herein designated VGAM HOST TARGET RNA. This complementary binding is due to the fact that the nucleotide sequence of VGAM258 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM258 RNA, herein designated VGAM RNA, may have a different number of host target binding sites in untranslated regions of a VGAM258 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM258 RNA, herein designated VGAM RNA, to host target binding sites on VGAM258 host target RNA, herein designated VGAM HOST TARGET RNA, such as BINDING SITE I, BINDING SITE II and BINDING SITE III, inhibits translation of VGAM258 host target RNA into VGAM258 host target protein, herein designated VGAM HOST TARGET PROTEIN. VGAM host target protein is therefore outlined by a broken line.

It is appreciated that VGAM258 host target gene, herein designated VGAM HOST TARGET GENE, in fact represents a plurality of VGAM258 host target genes. The mRNA of each one of this plurality of VGAM258 host target genes comprises one or more host target binding sites, each having a nucleotide sequence which is at least partly complementary to VGAM258 RNA, herein designated VGAM RNA, and which when bound by VGAM258 RNA causes inhibition of translation of respective one or more VGAM258 host target proteins.

It is further appreciated by one skilled in the art that the mode of translational inhibition illustrated by FIG. 1 with specific reference to translational inhibition exerted by VGAM258 gene, herein designated VGAM GENE, on one or more VGAM258 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM258 is inhibition of expression of host target genes, as part of a novel viral mechanism of attacking a host. Accordingly, utilities of VGAM258 include diagnosis, prevention and treatment of viral infection by Vaccinia Virus. Specific functions, and accordingly utilities, of VGAM258 correlate with, and may be deduced from, the identity of the host target genes which VGAM258 binds and inhibits, and the function of these host target genes, as elaborated hereinbelow.

Nucleotide sequences of the VGAM258 precursor RNA, herein designated VGAM PRECURSOR RNA, and of the ‘diced’ VGAM258 RNA, herein designated VGAM RNA, and a schematic representation of the secondary folding of VGAM258 folded precursor RNA, herein designated VGAM FOLDED PRECURSOR RNA, of VGAM258 are further described hereinbelow with reference to Table 1.

Nucleotide sequences of host target binding sites, such as BINDING SITE-I, BINDING SITE-II and BINDING SITE-III of FIG. 1, found on VGAM258 host target RNA, and schematic representation of the complementarity of each of these host target binding sites to VGAM258 RNA, herein designated VGAM RNA, are described hereinbelow with reference to Table 2.

As mentioned hereinabove with reference to FIG. 1, a function of VGAM258 gene, herein designated VGAM is inhibition of expression of VGAM258 target genes. It is appreciated that specific functions, and accordingly utilities, of VGAM258 correlate with, and may be deduced from, the identity of the target genes which VGAM258 binds and inhibits, and the function of these target genes, as elaborated hereinbelow.

Hypoxia Up-regulated 1 (HYOU1, Accession XM_{—}006464) is a VGAM258 host target gene. HYOU1 BINDING SITE is HOST TARGET binding site found in the 3′ untranslated region of mRNA encoded by HYOU1, corresponding to a HOST TARGET binding site such as BINDING SITE I, BINDING SITE II or BINDING SITE III. Table 2 illustrates the complementarity of the nucleotide sequences of HYOU1 BINDING SITE, designated SEQ ID:2537, to the nucleotide sequence of VGAM258 RNA, herein designated VGAM RNA, also designated SEQ ID:593.

A function of VGAM258 is therefore inhibition of Hypoxia Up-regulated 1 (HYOU1, Accession XM_{—}006464). Accordingly, utilities of VGAM258 include diagnosis, prevention and treatment of diseases and clinical conditions associated with HYOU1. Integrin, Beta 1 (fibronectin receptor, beta polypeptide, antigen CD29 includes MDF2, MSK12) (ITGB1, Accession NM_{—}002211) is another VGAM258 host target gene. ITGB1 BINDING SITE is HOST TARGET binding site found in the 3′ untranslated region of mRNA encoded by ITGB1, corresponding to a HOST TARGET binding site such as BINDING SITE I, BINDING SITE II or BINDING SITE III. Table 2 illustrates the complementarity of the nucleotide sequences of ITGB1 BINDING SITE, designated SEQ ID:910, to the nucleotide sequence of VGAM258 RNA, herein designated VGAM RNA, also designated SEQ ID:593.

Another function of VGAM258 is therefore inhibition of Integrin, Beta 1 (fibronectin receptor, beta polypeptide, antigen CD29 includes MDF2, MSK12) (ITGB1, Accession NM_{—}002211), a gene which acts as a fibronectin receptor. Accordingly, utilities of VGAM258 include diagnosis, prevention and treatment of diseases and clinical conditions associated with ITGB1. The function of ITGB1 and its association with various diseases and clinical conditions, has been established by previous studies, as described hereinabove with reference to VGAM154. Melanoma Antigen, Family A, 4 (MAGEA4, Accession NM_{—}002362) is another VGAM258 host target gene. MAGEA4 BINDING SITE is HOST TARGET binding site found in the 3′ untranslated region of mRNA encoded by MAGEA4, corresponding to a HOST TARGET binding site such as BINDING SITE I, BINDING SITE II or BINDING SITE III. Table 2 illustrates the complementarity of the nucleotide sequences of MAGEA4 BINDING SITE, designated SEQ ID:922, to the nucleotide sequence of VGAM258 RNA, herein designated VGAM RNA, also designated SEQ ID:593.

Another function of VGAM258 is therefore inhibition of Melanoma Antigen, Family A, 4 (MAGEA4, Accession NM_{—}002362), a gene which may play a role in embryonal development and tumor transformation or tumor progression. Accordingly, utilities of VGAM258 include diagnosis, prevention and treatment of diseases and clinical conditions associated with MAGEA4. The function of MAGEA4 has been established by previous studies. Genes of the MAGE-A family are expressed in several types of solid tumors but are silent in normal tissues, with the exception of male germline cells, which do not carry HLA molecules. Therefore, peptides encoded by MAGE-A genes are strictly tumor-specific antigens that can be recognized by CTL and constitute promising targets for immunotherapy. Chambost et al. (2000) found that 5 of 18 samples (28%) from patients with Hodgkin disease expressed MAGE-A4. In tissue sections, staining by a monoclonal antibody that recognizes MAGE-A4 protein was observed in 11 of 53 samples (21%) from patients with Hodgkin disease. In the positive samples, the Reed-Sternberg cells were strongly stained, whereas the surrounding cells were not. These results indicated that Hodgkin disease may be a target for specific immunotherapy involving MAGE-A4 antigens. De Plaen et al. (1994) used human/rodent cell hybrids to map the MAGE family A cluster to Xq26-qter. Rogner et al. (1995) refined the mapping of the MAGE family A cluster to Xq28. The 12 genes are arranged in 3 clusters within 3.5 Mb.

Full details of the abovementioned studies are described in the following publications, the disclosure of which are hereby incorporated by reference:
Chambost, H.; Van Baren, N.; Brasseur, F.; Godelaine, D.; Xerri, L.; Landi, S. J.; Theate, I.; Plumas, J.; Spagnoli, G. C.; Michel, G.; Coulie, P. G.; Olive, D.: Expression of gene MAGE-A4 in Reed-Sternberg cells. Blood 95: 3530-3533, 2000.; and
De Plaen, E.; Arden, K.; Traversari, C.; Gaforio, J. J.; Szikora, J.-P.; De Smet, C.; Brasseur, F.; van der Bruggen, P.; Lethe, B.; Lurquin, C.; Brasseur, R.; Chomez, P.; De Backer, O.;.

Further studies establishing the function and utilities of MAGEA4 are found in John Hopkins OMIM database record ID 300175, and in sited publications numbered 161 and 1640-1641 listed in the bibliography section hereinbelow, which are also hereby incorporated by reference. Pituitary Tumor-transforming 1 Interacting Protein (PTTG1IP, Accession NM_{—}004339) is another VGAM258 host target gene. PTTG1IP BINDING SITE is HOST TARGET binding site found in the 3′ untranslated region of mRNA encoded by PTTG1IP, corresponding to a HOST TARGET binding site such as BINDING SITE I, BINDING SITE II or BINDING SITE III. Table 2 illustrates the complementarity of the nucleotide sequences of PTTG1IP BINDING SITE, designated SEQ ID:1100, to the nucleotide sequence of VGAM258 RNA, herein designated VGAM RNA, also designated SEQ ID:593.

Another function of VGAM258 is therefore inhibition of Pituitary Tumor-transforming 1 Interacting Protein (PTTG1IP, Accession NM_{—}004339), a gene which facilitates the translocation of PTTG to the nucleus. Accordingly, utilities of VGAM258 include diagnosis, prevention and treatment of diseases and clinical conditions associated with PTTG1IP. The function of PTTG1IP has been established by previous studies. In the course of constructing a transcript map for chromosome 21, Yaspo et al. (1995) isolated numerous coding segments from 21q22.3, including transcription unit TU6. Yaspo et al. (1998) cloned cDNAs corresponding to TU6 by screening a human fetal thymus cDNA library with a partial cDNA and trapped exon from TU6. Homology searches of sequence databases using the translated sequence did not detect similarities to known proteins, and the authors named the novel gene C21ORF3. The predicted 180-amino acid C21ORF3 protein has features of a type 1a integral membrane protein and contains the tetrapeptide YXRF, a motif observed in proteins internalized via coated pit-mediated endocytosis. Northern blot analysis detected a 2.69-kb C21ORF3 mRNA in all tissues examined. Using a yeast 2-hybrid screen on a human testis cDNA library with rat pituitary tumor-transforming gene (PTTG; 604147) as bait, Chien and Pei (2000) isolated a cDNA encoding PTTG1IP, which they called PBF (PTTG-binding factor). Sequence analysis predicted that the 179-amino acid PBF protein, which is 92% identical to C21ORF3, contains multiple phosphorylation sites, 5 potential N— and O-glycosylation sites, a potential N-terminal sorting signal, and a C-terminal nuclear localization signal (NLS). Northern and dot blot analysis detected a 2.8-kb PBF transcript in all tissues tested, with highest expression in placenta. Pull-down and coimmunoprecipitation analyses showed that PBF and PTTG interact specifically via their C-terminal regions. Western blot analysis and immunofluorescence microscopy showed that whereas PTTG is expressed primarily in the cytoplasm, PBF is expressed in both the cytoplasm and nucleus. The authors demonstrated that PBF, via its NLS, facilitates the translocation of PTTG to the nucleus. Reporter assay analysis indicated that coexpression of PBF and PTTG induces transcription of basic fibroblast growth factor (FGF2; 134920).

Full details of the abovementioned studies are described in the following publications, the disclosure of which are hereby incorporated by reference:
Yaspo, M.-L.; Aaltonen, J.; Horelli-Kuitunen, N.; Peltonen, L.; Lehrach, H.: Cloning of a novel human putative type 1a integral membrane protein mapping to 21q22.3. Genomics 49: 133-136, 1998.; and
Yaspo, M.-L.; Gellen, L.; Mott, R.; Korn, B.; Nizetic, D.; Poustka, A. M.; Lehrach, H.: Model for a transcript map of human chromosome 21: isolation of new coding sequences from exon an.

Further studies establishing the function and utilities of PTTG IP are found in John Hopkins OMIM database record ID 603784, and in sited publications numbered 1867-1869 listed in the bibliography section hereinbelow, which are also hereby incorporated by reference. RAB27A, Member RAS Oncogene Family (RAB27A, Accession NM_{—}004580) is another VGAM258 host target gene. RAB27A BINDING SITE is HOST TARGET binding site found in the 3′ untranslated region of mRNA encoded by RAB27A, corresponding to a HOST TARGET binding site such as BINDING SITE I, BINDING SITE II or BINDING SITE III. Table 2 illustrates the complementarity of the nucleotide sequences of RAB27A BINDING SITE, designated SEQ ID:1129, to the nucleotide sequence of VGAM258 RNA, herein designated VGAM RNA, also designated SEQ ID:593.

Another function of VGAM258 is therefore inhibition of RAB27A, Member RAS Oncogene Family (RAB27A, Accession NM_{—}004580). Accordingly, utilities of VGAM258 include diagnosis, prevention and treatment of diseases and clinical conditions associated with RAB27A. Sal-like 2 (Drosophila) (SALL2, Accession XM_{—}033473) is another VGAM258 host target gene. SALL2 BINDING SITE is HOST TARGET binding site found in the 5′ untranslated region of mRNA encoded by SALL2, corresponding to a HOST TARGET binding site such as BINDING SITE I, BINDING SITE II or BINDING SITE III. Table 2 illustrates the complementarity of the nucleotide sequences of SALL2 BINDING SITE, designated SEQ ID:2671, to the nucleotide sequence of VGAM258 RNA, herein designated VGAM RNA, also designated SEQ ID:593.

Another function of VGAM258 is therefore inhibition of Sal-like 2 (Drosophila) (SALL2, Accession XM_{—}033473). Accordingly, utilities of VGAM258 include diagnosis, prevention and treatment of diseases and clinical conditions associated with SALL2. Splicing Factor, Arginine/serine-rich 7, 35 kDa (SFRS7, Accession XM_{—}002575) is another VGAM258 host target gene. SFRS7 BINDING SITE is HOST TARGET binding site found in the 3′ untranslated region of mRNA encoded by SFRS7, corresponding to a HOST TARGET binding site such as BINDING SITE I, BINDING SITE II or BINDING SITE III. Table 2 illustrates the complementarity of the nucleotide sequences of SFRS7 BINDING SITE, designated SEQ ID:2529, to the nucleotide sequence of VGAM258 RNA, herein designated VGAM RNA, also designated SEQ ID:593.

Another function of VGAM258 is therefore inhibition of Splicing Factor, Arginine/serine-rich 7, 35 kDa (SFRS7, Accession XM_{—}002575), a gene which is required for premnra splicing. Accordingly, utilities of VGAM258 include diagnosis, prevention and treatment of diseases and clinical conditions associated with SFRS7. The function of SFRS7 has been established by previous studies. Cavaloc et al. (1994) used a monoclonal antibody to identify a splicing factor of 35 kD which they named 9G8. Based on partial sequence of tryptic peptides, the authors designed degenerate PCR primers and obtained a PCR product which was used to probe genomic and cDNA libraries. The isolation and characterization of cDNA clones indicated that the 9G8 protein (gene symbol, SFRS7) is a member of the serine/arginine (SR) splicing factor family because it includes an N-terminal RNA binding domain and a C-terminal SR domain. Members of this family are thought to play key roles in alternative splicing. The RNA binding domain of 9G8 is closely related (79 to 71% identity) to those of the SR factors human SRp20 (OMIM Ref. No. 603364) and Drosophila RBP1. Immunodepletion of the 9G8 protein from a nuclear extract resulted in the loss of splicing activity. In turn, in vitro-expressed recombinant 9G8 protein rescued the splicing activity of a 9G8-depleted nuclear extract. Popielarz et al. (1995) isolated and characterized the human 9G8 gene. The gene spans 7,745 bp and consists of 8 exons and 7 introns within the coding sequence, thus contrasting with the organization of some other genes of the SR splicing factor family. By isotopic in situ hybridization, they localized the gene to 2p22-p21. The 5-prime flanking region is GC-rich and contains basal promoter sequences and potential regulatory elements. They presented results raising the possibility that alternative splicing of intron 3 provides a mechanism for modulation of the 9G8 function.

Full details of the abovementioned studies are described in the following publications, the disclosure of which are hereby incorporated by reference:
Cavaloc, Y.; Popielarz, M.; Fuchs, J.-P.; Gattoni, R.; Stevenin, J.: Characterization and cloning of the human splicing factor 9G8: a novel 35 kDa factor of the serine/arginine protein family. EMBO J. 13: 2639-2649, 1994.; and
Popielarz, M.; Cavaloc, Y.; Mattei, M.-G.; Gattoni, R.; Stevenin, J.: The gene encoding human splicing factor 9G8: structure, chromosomal localization, and expression of alternatively pro.

Further studies establishing the function and utilities of SFRS7 are found in John Hopkins OMIM database record ID 600572, and in sited publications numbered 2151-2152 listed in the bibliography section hereinbelow, which are also hereby incorporated by reference. Solute Carrier Family 19 (thiamine transporter), Member 2 (SLC19A2, Accession XM_{—}044421) is another VGAM258 host target gene. SLC19A2 BINDING SITE is HOST TARGET binding site found in the 3′ untranslated region of mRNA encoded by SLC19A2, corresponding to a HOST TARGET binding site such as BINDING SITE I, BINDING SITE II or BINDING SITE III. Table 2 illustrates the complementarity of the nucleotide sequences of SLC19A2 BINDING SITE, designated SEQ ID:2836, to the nucleotide sequence of VGAM258 RNA, herein designated VGAM RNA, also designated SEQ ID:593.

Another function of VGAM258 is therefore inhibition of Solute Carrier Family 19 (thiamine transporter), Member 2 (SLC19A2, Accession XM_{—}044421). Accordingly, utilities of VGAM258 include diagnosis, prevention and treatment of diseases and clinical conditions associated with SLC19A2. Chromosome 20 Open Reading Frame 97 (C20orf97, Accession NM_{—}021158) is another VGAM258 host target gene. C20orf97 BINDING SITE is HOST TARGET binding site found in the 3′ untranslated region of mRNA encoded by C20orf97, corresponding to a HOST TARGET binding site such as BINDING SITE I, BINDING SITE II or BINDING SITE III. Table 2 illustrates the complementarity of the nucleotide sequences of C20orf97 BINDING SITE, designated SEQ ID: 1946, to the nucleotide sequence of VGAM258 RNA, herein designated VGAM RNA, also designated SEQ ID:593.

Another function of VGAM258 is therefore inhibition of Chromosome 20 Open Reading Frame 97 (C20orf97, Accession NM_{—}021158). Accordingly, utilities of VGAM258 include diagnosis, prevention and treatment of diseases and clinical conditions associated with C20orf97. CYB5-M (Accession NM_{—}030579) is another VGAM258 host target gene. CYB5-M BINDING SITE1 and CYB5-M BINDING SITE2 are HOST TARGET binding sites found in untranslated regions of mRNA encoded by CYB5-M, corresponding to HOST TARGET binding sites such as BINDING SITE I, BINDING SITE II or BINDING SITE III. Table 2 illustrates the complementarity of the nucleotide sequences of CYB5-M BINDING SITE1 and CYB5-M BINDING SITE2, designated SEQ ID:2150 and SEQ ID:3658 respectively, to the nucleotide sequence of VGAM258 RNA, herein designated VGAM RNA, also designated SEQ ID:593.

Another function of VGAM258 is therefore inhibition of CYB5-M (Accession NM_{—}030579). Accordingly, utilities of VGAM258 include diagnosis, prevention and treatment of diseases and clinical conditions associated with CYB5-M. FLJ10853 (Accession NM_{—}018246) is another VGAM258 host target gene. FLJ10853 BINDING SITE is HOST TARGET binding site found in the 5′ untranslated region of mRNA encoded by FLJ10853, corresponding to a HOST TARGET binding site such as BINDING SITE I, BINDING SITE II or BINDING SITE III. Table 2 illustrates the complementarity of the nucleotide sequences of FLJ10853 BINDING SITE, designated SEQ ID:1804, to the nucleotide sequence of VGAM258 RNA, herein designated VGAM RNA, also designated SEQ ID:593.

Another function of VGAM258 is therefore inhibition of FLJ10853 (Accession NM_{—}018246). Accordingly, utilities of VGAM258 include diagnosis, prevention and treatment of diseases and clinical conditions associated with FLJ10853. FLJ14327 (Accession NM_{—}024912) is another VGAM258 host target gene. FLJ14327 BINDING SITE is HOST TARGET binding site found in the 3′ untranslated region of mRNA encoded by FLJ14327, corresponding to a HOST TARGET binding site such as BINDING SITE I, BINDING SITE II or BINDING SITE III. Table 2 illustrates the complementarity of the nucleotide sequences of FLJ14327 BINDING SITE, designated SEQ ID:2111, to the nucleotide sequence of VGAM258 RNA, herein designated VGAM RNA, also designated SEQ ID:593.

Another function of VGAM258 is therefore inhibition of FLJ14327 (Accession NM_{—}024912). Accordingly, utilities of VGAM258 include diagnosis, prevention and treatment of diseases and clinical conditions associated with FLJ14327. FLJ14681 (Accession NM_{—}032824) is another VGAM258 host target gene. FLJ14681 BINDING SITE is HOST TARGET binding site found in the 5′ untranslated region of mRNA encoded by FLJ14681, corresponding to a HOST TARGET binding site such as BINDING SITE I, BINDING SITE II or BINDING SITE III. Table 2 illustrates the complementarity of the nucleotide sequences of FLJ14681 BINDING SITE, designated SEQ ID:2285, to the nucleotide sequence of VGAM258 RNA, herein designated VGAM RNA, also designated SEQ ID:593.

Another function of VGAM258 is therefore inhibition of FLJ14681 (Accession NM_{—}032824). Accordingly, utilities of VGAM258 include diagnosis, prevention and treatment of diseases and clinical conditions associated with FLJ14681. Heterogeneous Nuclear Ribonucleoprotein A3 (HNRPA3, Accession NM_{—}005758) is another VGAM258 host target gene. HNRPA3 BINDING SITE is HOST TARGET binding site found in the 3′ untranslated region of mRNA encoded by HNRPA3, corresponding to a HOST TARGET binding site such as BINDING SITE I, BINDING SITE II or BINDING SITE III. Table 2 illustrates the complementarity of the nucleotide sequences of HNRPA3 BINDING SITE, designated SEQ ID:1245, to the nucleotide sequence of VGAM258 RNA, herein designated VGAM RNA, also designated SEQ ID:593.

Another function of VGAM258 is therefore inhibition of Heterogeneous Nuclear Ribonucleoprotein A3 (HNRPA3, Accession NM_{—}005758). Accordingly, utilities of VGAM258 include diagnosis, prevention and treatment of diseases and clinical conditions associated with HNRPA3. KIAA0426 (Accession NM_{—}014724) is another VGAM258 host target gene. KIAA0426 BINDING SITE is HOST TARGET binding site found in the 3′ untranslated region of mRNA encoded by KIAA0426, corresponding to a HOST TARGET binding site such as BINDING SITE I, BINDING SITE II or BINDING SITE III. Table 2 illustrates the complementarity of the nucleotide sequences of KIAA0426 BINDING SITE, designated SEQ ID: 1532, to the nucleotide sequence of VGAM258 RNA, herein designated VGAM RNA, also designated SEQ ID:593.

Another function of VGAM258 is therefore inhibition of KIAA0426 (Accession NM_{—}014724). Accordingly, utilities of VGAM258 include diagnosis, prevention and treatment of diseases and clinical conditions associated with KIAA0426. KIAA0594 (Accession XM_{—}036117) is another VGAM258 host target gene. KIAA0594 BINDING SITE is HOST TARGET binding site found in the 3′ untranslated region of mRNA encoded by KIAA0594, corresponding to a HOST TARGET binding site such as BINDING SITE I, BINDING SITE II or BINDING SITE III. Table 2 illustrates the complementarity of the nucleotide sequences of KIAA0594 BINDING SITE, designated SEQ ID:2707, to the nucleotide sequence of VGAM258 RNA, herein designated VGAM RNA, also designated SEQ ID:593.

Another function of VGAM258 is therefore inhibition of KIAA0594 (Accession XM_{—}036117). Accordingly, utilities of VGAM258 include diagnosis, prevention and treatment of diseases and clinical conditions associated with KIAA0594. KIAA0635 (Accession NM_{—}014645) is another VGAM258 host target gene. KIAA0635 BINDING SITE is HOST TARGET binding site found in the 5′ untranslated region of mRNA encoded by KIAA0635, corresponding to a HOST TARGET binding site such as BINDING SITE I, BINDING SITE II or BINDING SITE III. Table 2 illustrates the complementarity of the nucleotide sequences of KIAA0635 BINDING SITE, designated SEQ ID:1513, to the nucleotide sequence of VGAM258 RNA, herein designated VGAM RNA, also designated SEQ ID:593.

Another function of VGAM258 is therefore inhibition of KIAA0635 (Accession NM_{—}014645). Accordingly, utilities of VGAM258 include diagnosis, prevention and treatment of diseases and clinical conditions associated with KIAA0635. PDZ Domain Containing 2 (PDZD2, Accession XM_{—}087705) is another VGAM258 host target gene. PDZD2 BINDING SITE is HOST TARGET binding site found in the 5′ untranslated region of mRNA encoded by PDZD2, corresponding to a HOST TARGET binding site such as BINDING SITE I, BINDING SITE II or BINDING SITE III. Table 2 illustrates the complementarity of the nucleotide sequences of PDZD2 BINDING SITE, designated SEQ ID:3181, to the nucleotide sequence of VGAM258 RNA, herein designated VGAM RNA, also designated SEQ ID:593.

Another function of VGAM258 is therefore inhibition of PDZ Domain Containing 2 (PDZD2, Accession XM_{—}087705). Accordingly, utilities of VGAM258 include diagnosis, prevention and treatment of diseases and clinical conditions associated with PDZD2. PP1628 (Accession NM_{—}025201) is another VGAM258 host target gene. PP1628 BINDING SITE is HOST TARGET binding site found in the 5′ untranslated region of mRNA encoded by PP1628, corresponding to a HOST TARGET binding site such as BINDING SITE I, BINDING SITE II or BINDING SITE III. Table 2 illustrates the complementarity of the nucleotide sequences of PP1628 BINDING SITE, designated SEQ ID:2145, to the nucleotide sequence of VGAM258 RNA, herein designated VGAM RNA, also designated SEQ ID:593.

Another function of VGAM258 is therefore inhibition of PP1628 (Accession NM_{—}025201). Accordingly, utilities of VGAM258 include diagnosis, prevention and treatment of diseases and clinical conditions associated with PP1628. TIP120A (Accession NM_{—}018448) is another VGAM258 host target gene. TIP120A BINDING SITE is HOST TARGET binding site found in the 3′ untranslated region of mRNA encoded by TIP120A, corresponding to a HOST TARGET binding site such as BINDING SITE I, BINDING SITE II or BINDING SITE III. Table 2 illustrates the complementarity of the nucleotide sequences of TIP120A BINDING SITE, designated SEQ ID:1829, to the nucleotide sequence of VGAM258 RNA, herein designated VGAM RNA, also designated SEQ ID:593.

Another function of VGAM258 is therefore inhibition of TIP120A (Accession NM_{—}018448). Accordingly, utilities of VGAM258 include diagnosis, prevention and treatment of diseases and clinical conditions associated with TIP120A. LOC220988 (Accession XM_{—}165561) is another VGAM258 host target gene. LOC220988 BINDING SITE is HOST TARGET binding site found in the 3′ untranslated region of mRNA encoded by LOC220988, corresponding to a HOST TARGET binding site such as BINDING SITE I, BINDING SITE II or BINDING SITE III. Table 2 illustrates the complementarity of the nucleotide sequences of LOC220988 BINDING SITE, designated SEQ ID:3501, to the nucleotide sequence of VGAM258 RNA, herein designated VGAM RNA, also designated SEQ ID:593.

Another function of VGAM258 is therefore inhibition of LOC220988 (Accession XM_{—}165561). Accordingly, utilities of VGAM258 include diagnosis, prevention and treatment of diseases and clinical conditions associated with LOC220988. LOC221337 (Accession XM_{—}166387) is another VGAM258 host target gene. LOC221337 BINDING SITE is HOST TARGET binding site found in the 5′ untranslated region of mRNA encoded by LOC221337, corresponding to a HOST TARGET binding site such as BINDING SITE I, BINDING SITE II or BINDING SITE III. Table 2 illustrates the complementarity of the nucleotide sequences of LOC221337 BINDING SITE, designated SEQ ID:3555, to the nucleotide sequence of VGAM258 RNA, herein designated VGAM RNA, also designated SEQ ID:593.

Another function of VGAM258 is therefore inhibition of LOC221337 (Accession XM_{—}166387). Accordingly, utilities of VGAM258 include diagnosis, prevention and treatment of diseases and clinical conditions associated with LOC221337. LOC257017 (Accession XM_{—}173227) is another VGAM258 host target gene. LOC257017 BINDING SITE is HOST TARGET binding site found in the 5′ untranslated region of mRNA encoded by LOC257017, corresponding to a HOST TARGET binding site such as BINDING SITE I, BINDING SITE II or BINDING SITE III. Table 2 illustrates the complementarity of the nucleotide sequences of LOC257017 BINDING SITE, designated SEQ ID:3737, to the nucleotide sequence of VGAM258 RNA, herein designated VGAM RNA, also designated SEQ ID:593.

Another function of VGAM258 is therefore inhibition of LOC257017 (Accession XM_{—}173227). Accordingly, utilities of VGAM258 include diagnosis, prevention and treatment of diseases and clinical conditions associated with LOC257017. LOC90829 (Accession XM_{—}034325) is another VGAM258 host target gene. LOC90829 BINDING SITE is HOST TARGET binding site found in the 5′ untranslated region of mRNA encoded by LOC90829, corresponding to a HOST TARGET binding site such as BINDING SITE I, BINDING SITE II or BINDING SITE III. Table 2 illustrates the complementarity of the nucleotide sequences of LOC90829 BINDING SITE, designated SEQ ID:2680, to the nucleotide sequence of VGAM258 RNA, herein designated VGAM RNA, also designated SEQ ID:593.

Another function of VGAM258 is therefore inhibition of LOC90829 (Accession XM_{—}034325). Accordingly, utilities of VGAM258 include diagnosis, prevention and treatment of diseases and clinical conditions associated with LOC90829. FIG. 1 further provides a conceptual description of a novel bioinformatically detected viral gene of the present invention, referred to here as Viral Genomic Address Messenger 259 (VGAM259) viral gene, which modulates expression of respective host target genes thereof, the function and utility of which host target genes is known in the art.

VGAM259 is a novel bioinformatically detected regulatory, non protein coding, viral micro RNA (miRNA) gene. The method by which VGAM259 was detected is described hereinabove with reference to FIGS. 1-8.

VGAM259 gene, herein designated VGAM GENE, is a viral gene contained in the genome of Vaccinia Virus. VGAM259 host target gene, herein designated VGAM HOST TARGET GENE, is a human gene contained in the human genome.

VGAM259 gene encodes a VGAM259 precursor RNA, herein designated VGAM PRECURSOR RNA. Similar to other miRNA genes, and unlike most ordinary genes, VGAM259 precursor RNA does not encode a protein. A nucleotide sequence identical or highly similar to the nucleotide sequence of VGAM259 precursor RNA is designated SEQ ID:245, and is provided hereinbelow with reference to the sequence listing part. Nucleotide sequence SEQ ID:245 is located at position 144258 relative to the genome of Vaccinia Virus.

VGAM259 precursor RNA folds onto itself, forming VGAM259 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM259 folded precursor RNA into VGAM259 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 25%) nucleotide sequence of VGAM259 RNA is designated SEQ ID:594, and is provided hereinbelow with reference to the sequence listing part.

VGAM259 host target gene, herein designated VGAM HOST TARGET GENE, encodes a corresponding messenger RNA, VGAM259 host target RNA, herein designated VGAM HOST TARGET RNA. VGAM259 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM259 RNA, herein designated VGAM RNA, binds complementarily to one or more host target binding sites located in untranslated regions of VGAM259 host target RNA, herein designated VGAM HOST TARGET RNA. This complementary binding is due to the fact that the nucleotide sequence of VGAM259 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM259 RNA, herein designated VGAM RNA, may have a different number of host target binding sites in untranslated regions of a VGAM259 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM259 RNA, herein designated VGAM RNA, to host target binding sites on VGAM259 host target RNA, herein designated VGAM HOST TARGET RNA, such as BINDING SITE I, BINDING SITE II and BINDING SITE III, inhibits translation of VGAM259 host target RNA into VGAM259 host target protein, herein designated VGAM HOST TARGET PROTEIN. VGAM host target protein is therefore outlined by a broken line.

It is appreciated that VGAM259 host target gene, herein designated VGAM HOST TARGET GENE, in fact represents a plurality of VGAM259 host target genes. The mRNA of each one of this plurality of VGAM259 host target genes comprises one or more host target binding sites, each having a nucleotide sequence which is at least partly complementary to VGAM259 RNA, herein designated VGAM RNA, and which when bound by VGAM259 RNA causes inhibition of translation of respective one or more VGAM259 host target proteins.

It is further appreciated by one skilled in the art that the mode of translational inhibition illustrated by FIG. 1 with specific reference to translational inhibition exerted by VGAM259 gene, herein designated VGAM GENE, on one or more VGAM259 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM259 is inhibition of expression of host target genes, as part of a novel viral mechanism of attacking a host. Accordingly, utilities of VGAM259 include diagnosis, prevention and treatment of viral infection by Vaccinia Virus. Specific functions, and accordingly utilities, of VGAM259 correlate with, and may be deduced from, the identity of the host target genes which VGAM259 binds and inhibits, and the function of these host target genes, as elaborated hereinbelow.

Nucleotide sequences of the VGAM259 precursor RNA, herein designated VGAM PRECURSOR RNA, and of the ‘diced’ VGAM259 RNA, herein designated VGAM RNA, and a schematic representation of the secondary folding of VGAM259 folded precursor RNA, herein designated VGAM FOLDED PRECURSOR RNA, of VGAM259 are further described hereinbelow with reference to Table 1.

Nucleotide sequences of host target binding sites, such as BINDING SITE-I, BINDING SITE-II and BINDING SITE-III of FIG. 1, found on VGAM259 host target RNA, and schematic representation of the complementarity of each of these host target binding sites to VGAM259 RNA, herein designated VGAM RNA, are described hereinbelow with reference to Table 2.

As mentioned hereinabove with reference to FIG. 1, a function of VGAM259 gene, herein designated VGAM is inhibition of expression of VGAM259 target genes. It is appreciated that specific functions, and accordingly utilities, of VGAM259 correlate with, and may be deduced from, the identity of the target genes which VGAM259 binds and inhibits, and the function of these target genes, as elaborated hereinbelow.

KIAA0268 (Accession XM_{—}046126) is a VGAM259 host target gene. KIAA0268 BINDING SITE is HOST TARGET binding site found in the 3′ untranslated region of mRNA encoded by KIAA0268, corresponding to a HOST TARGET binding site such as BINDING SITE I, BINDING SITE II or BINDING SITE III. Table 2 illustrates the complementarity of the nucleotide sequences of KIAA0268 BINDING SITE, designated SEQ ID:2874, to the nucleotide sequence of VGAM259 RNA, herein designated VGAM RNA, also designated SEQ ID:594.

A function of VGAM259 is therefore inhibition of KIAA0268 (Accession XM_{—}046126). Accordingly, utilities of VGAM259 include diagnosis, prevention and treatment of diseases and clinical conditions associated with KIAA0268. KIAA1255 (Accession XM_{—}040626) is another VGAM259 host target gene. KIAA1255 BINDING SITE is HOST TARGET binding site found in the 3′ untranslated region of mRNA encoded by KIAA1255, corresponding to a HOST TARGET binding site such as BINDING SITE I, BINDING SITE II or BINDING SITE III. Table 2 illustrates the complementarity of the nucleotide sequences of KIAA1255 BINDING SITE, designated SEQ ID:2783, to the nucleotide sequence of VGAM259 RNA, herein designated VGAM RNA, also designated SEQ ID:594.

Another function of VGAM259 is therefore inhibition of KIAA1255 (Accession XM_{—}040626). Accordingly, utilities of VGAM259 include diagnosis, prevention and treatment of diseases and clinical conditions associated with KIAA1255. MGC5370 (Accession NM_{—}032739) is another VGAM259 host target gene. MGC5370 BINDING SITE is HOST TARGET binding site found in the 3′ untranslated region of mRNA encoded by MGC5370, corresponding to a HOST TARGET binding site such as BINDING SITE I, BINDING SITE II or BINDING SITE III. Table 2 illustrates the complementarity of the nucleotide sequences of MGC5370 BINDING SITE, designated SEQ ID:2275, to the nucleotide sequence of VGAM259 RNA, herein designated VGAM RNA, also designated SEQ ID:594.

Another function of VGAM259 is therefore inhibition of MGC5370 (Accession NM_{—}032739). Accordingly, utilities of VGAM259 include diagnosis, prevention and treatment of diseases and clinical conditions associated with MGC5370. UK114 (Accession NM_{—}005836) is another VGAM259 host target gene. UK114 BINDING SITE is HOST TARGET binding site found in the 3′ untranslated region of mRNA encoded by UK114, corresponding to a HOST TARGET binding site such as BINDING SITE I, BINDING SITE II or BINDING SITE III. Table 2 illustrates the complementarity of the nucleotide sequences of UK114 BINDING SITE, designated SEQ ID:1255, to the nucleotide sequence of VGAM259 RNA, herein designated VGAM RNA, also designated SEQ ID:594.

Another function of VGAM259 is therefore inhibition of UK114 (Accession NM_{—}005836). Accordingly, utilities of VGAM259 include diagnosis, prevention and treatment of diseases and clinical conditions associated with UK114. FIG. 1 further provides a conceptual description of a novel bioinformatically detected viral gene of the present invention, referred to here as Viral Genomic Address Messenger 260 (VGAM260) viral gene, which modulates expression of respective host target genes thereof, the function and utility of which host target genes is known in the art.

VGAM260 is a novel bioinformatically detected regulatory, non protein coding, viral micro RNA (miRNA) gene. The method by which VGAM260 was detected is described hereinabove with reference to FIGS. 1-8.

VGAM260 gene, herein designated VGAM GENE, is a viral gene contained in the genome of Vaccinia Virus. VGAM260 host target gene, herein designated VGAM HOST TARGET GENE, is a human gene contained in the human genome.

VGAM260 gene encodes a VGAM260 precursor RNA, herein designated VGAM PRECURSOR RNA. Similar to other miRNA genes, and unlike most ordinary genes, VGAM260 precursor RNA does not encode a protein. A nucleotide sequence identical or highly similar to the nucleotide sequence of VGAM260 precursor RNA is designated SEQ ID:246, and is provided hereinbelow with reference to the sequence listing part. Nucleotide sequence SEQ ID:246 is located at position 145827 relative to the genome of Vaccinia Virus.

VGAM260 precursor RNA folds onto itself, forming VGAM260 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM260 folded precursor RNA into VGAM260 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 89%) nucleotide sequence of VGAM260 RNA is designated SEQ ID:595, and is provided hereinbelow with reference to the sequence listing part.

VGAM260 host target gene, herein designated VGAM HOST TARGET GENE, encodes a corresponding messenger RNA, VGAM260 host target RNA, herein designated VGAM HOST TARGET RNA. VGAM260 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM260 RNA, herein designated VGAM RNA, binds complementarily to one or more host target binding sites located in untranslated regions of VGAM260 host target RNA, herein designated VGAM HOST TARGET RNA. This complementary binding is due to the fact that the nucleotide sequence of VGAM260 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM260 RNA, herein designated VGAM RNA, may have a different number of host target binding sites in untranslated regions of a VGAM260 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM260 RNA, herein designated VGAM RNA, to host target binding sites on VGAM260 host target RNA, herein designated VGAM HOST TARGET RNA, such as BINDING SITE I, BINDING SITE II and BINDING SITE III, inhibits translation of VGAM260 host target RNA into VGAM260 host target protein, herein designated VGAM HOST TARGET PROTEIN. VGAM host target protein is therefore outlined by a broken line.

It is appreciated that VGAM260 host target gene, herein designated VGAM HOST TARGET GENE, in fact represents a plurality of VGAM260 host target genes. The mRNA of each one of this plurality of VGAM260 host target genes comprises one or more host target binding sites, each having a nucleotide sequence which is at least partly complementary to VGAM260 RNA, herein designated VGAM RNA, and which when bound by VGAM260 RNA causes inhibition of translation of respective one or more VGAM260 host target proteins.

It is further appreciated by one skilled in the art that the mode of translational inhibition illustrated by FIG. 1 with specific reference to translational inhibition exerted by VGAM260 gene, herein designated VGAM GENE, on one or more VGAM260 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM260 is inhibition of expression of host target genes, as part of a novel viral mechanism of attacking a host. Accordingly, utilities of VGAM260 include diagnosis, prevention and treatment of viral infection by Vaccinia Virus. Specific functions, and accordingly utilities, of VGAM260 correlate with, and may be deduced from, the identity of the host target genes which VGAM260 binds and inhibits, and the function of these host target genes, as elaborated hereinbelow.

Nucleotide sequences of the VGAM260 precursor RNA, herein designated VGAM PRECURSOR RNA, and of the ‘diced’ VGAM260 RNA, herein designated VGAM RNA, and a schematic representation of the secondary folding of VGAM260 folded precursor RNA, herein designated VGAM FOLDED PRECURSOR RNA, of VGAM260 are further described hereinbelow with reference to Table 1.

Nucleotide sequences of host target binding sites, such as BINDING SITE-I, BINDING SITE-II and BINDING SITE-III of FIG. 1, found on VGAM260 host target RNA, and schematic representation of the complementarity of each of these host target binding sites to VGAM260 RNA, herein designated VGAM RNA, are described hereinbelow with reference to Table 2.

As mentioned hereinabove with reference to FIG. 1, a function of VGAM260 gene, herein designated VGAM is inhibition of expression of VGAM260 target genes. It is appreciated that specific functions, and accordingly utilities, of VGAM260 correlate with, and may be deduced from, the identity of the target genes which VGAM260 binds and inhibits, and the function of these target genes, as elaborated hereinbelow.

Oxysterol Binding Protein-like 8 (OSBPL8, Accession NM_{—}020841) is a VGAM260 host target gene. OSBPL8 BINDING SITE is HOST TARGET binding site found in the 3′ untranslated region of mRNA encoded by OSBPL8, corresponding to a HOST TARGET binding site such as BINDING SITE I, BINDING SITE II or BINDING SITE III. Table 2 illustrates the complementarity of the nucleotide sequences of OSBPL8 BINDING SITE, designated SEQ ID:1927, to the nucleotide sequence of VGAM260 RNA, herein designated VGAM RNA, also designated SEQ ID:595.

A function of VGAM260 is therefore inhibition of Oxysterol Binding Protein-like 8 (OSBPL8, Accession NM_{—}020841). Accordingly, utilities of VGAM260 include diagnosis, prevention and treatment of diseases and clinical conditions associated with OSBPL8. LOC147463 (Accession XM_{—}085799) is another VGAM260 host target gene. LOC147463 BINDING SITE is HOST TARGET binding site found in the 3′ untranslated region of mRNA encoded by LOC147463, corresponding to a HOST TARGET binding site such as BINDING SITE I, BINDING SITE II or BINDING SITE III. Table 2 illustrates the complementarity of the nucleotide sequences of LOC147463 BINDING SITE, designated SEQ ID:3109, to the nucleotide sequence of VGAM260 RNA, herein designated VGAM RNA, also designated SEQ ID:595.

Another function of VGAM260 is therefore inhibition of LOC147463 (Accession XM_{—}085799). Accordingly, utilities of VGAM260 include diagnosis, prevention and treatment of diseases and clinical conditions associated with LOC147463. LOC151201 (Accession XM_{—}098021) is another VGAM260 host target gene. LOC151201 BINDING SITE is HOST TARGET binding site found in the 5′ untranslated region of mRNA encoded by LOC151201, corresponding to a HOST TARGET binding site such as BINDING SITE I, BINDING SITE II or BINDING SITE III. Table 2 illustrates the complementarity of the nucleotide sequences of LOC151201 BINDING SITE, designated SEQ ID:3340, to the nucleotide sequence of VGAM260 RNA, herein designated VGAM RNA, also designated SEQ ID:595.

Another function of VGAM260 is therefore inhibition of LOC151201 (Accession XM_{—}098021). Accordingly, utilities of VGAM260 include diagnosis, prevention and treatment of diseases and clinical conditions associated with LOC151201. FIG. 1 further provides a conceptual description of a novel bioinformatically detected viral gene of the present invention, referred to here as Viral Genomic Address Messenger 261 (VGAM261) viral gene, which modulates expression of respective host target genes thereof, the function and utility of which host target genes is known in the art.

VGAM261 is a novel bioinformatically detected regulatory, non protein coding, viral micro RNA (miRNA) gene. The method by which VGAM261 was detected is described hereinabove with reference to FIGS. 1-8.

VGAM261 gene, herein designated VGAM GENE, is a viral gene contained in the genome of Vaccinia Virus. VGAM261 host target gene, herein designated VGAM HOST TARGET GENE, is a human gene contained in the human genome.

VGAM261 gene encodes a VGAM261 precursor RNA, herein designated VGAM PRECURSOR RNA. Similar to other miRNA genes, and unlike most ordinary genes, VGAM261 precursor RNA does not encode a protein. A nucleotide sequence identical or highly similar to the nucleotide sequence of VGAM261 precursor RNA is designated SEQ ID:247, and is provided hereinbelow with reference to the sequence listing part. Nucleotide sequence SEQ ID:247 is located at position 145163 relative to the genome of Vaccinia Virus.

VGAM261 precursor RNA folds onto itself, forming VGAM261 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM261 folded precursor RNA into VGAM261 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM261 RNA is designated SEQ ID:596, and is provided hereinbelow with reference to the sequence listing part.

VGAM261 host target gene, herein designated VGAM HOST TARGET GENE, encodes a corresponding messenger RNA, VGAM261 host target RNA, herein designated VGAM HOST TARGET RNA. VGAM261 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM261 RNA, herein designated VGAM RNA, binds complementarily to one or more host target binding sites located in untranslated regions of VGAM261 host target RNA, herein designated VGAM HOST TARGET RNA. This complementary binding is due to the fact that the nucleotide sequence of VGAM261 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM261 RNA, herein designated VGAM RNA, may have a different number of host target binding sites in untranslated regions of a VGAM261 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM261 RNA, herein designated VGAM RNA, to host target binding sites on VGAM261 host target RNA, herein designated VGAM HOST TARGET RNA, such as BINDING SITE I, BINDING SITE II and BINDING SITE III, inhibits translation of VGAM261 host target RNA into VGAM261 host target protein, herein designated VGAM HOST TARGET PROTEIN. VGAM host target protein is therefore outlined by a broken line.

It is appreciated that VGAM261 host target gene, herein designated VGAM HOST TARGET GENE, in fact represents a plurality of VGAM261 host target genes. The mRNA of each one of this plurality of VGAM261 host target genes comprises one or more host target binding sites, each having a nucleotide sequence which is at least partly complementary to VGAM261 RNA, herein designated VGAM RNA, and which when bound by VGAM261 RNA causes inhibition of translation of respective one or more VGAM261 host target proteins.

It is further appreciated by one skilled in the art that the mode of translational inhibition illustrated by FIG. 1 with specific reference to translational inhibition exerted by VGAM261 gene, herein designated VGAM GENE, on one or more VGAM261 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM261 is inhibition of expression of host target genes, as part of a novel viral mechanism of attacking a host. Accordingly, utilities of VGAM261 include diagnosis, prevention and treatment of viral infection by Vaccinia Virus. Specific functions, and accordingly utilities, of VGAM261 correlate with, and may be deduced from, the identity of the host target genes which VGAM261 binds and inhibits, and the function of these host target genes, as elaborated hereinbelow.

Nucleotide sequences of the VGAM261 precursor RNA, herein designated VGAM PRECURSOR RNA, and of the ‘diced’ VGAM261 RNA, herein designated VGAM RNA, and a schematic representation of the secondary folding of VGAM261 folded precursor RNA, herein designated VGAM FOLDED PRECURSOR RNA, of VGAM261 are further described hereinbelow with reference to Table 1.

Nucleotide sequences of host target binding sites, such as BINDING SITE-I, BINDING SITE-II and BINDING SITE-III of FIG. 1, found on VGAM261 host target RNA, and schematic representation of the complementarity of each of these host target binding sites to VGAM261 RNA, herein designated VGAM RNA, are described hereinbelow with reference to Table 2.

As mentioned hereinabove with reference to FIG. 1, a function of VGAM261 gene, herein designated VGAM is inhibition of expression of VGAM261 target genes. It is appreciated that specific functions, and accordingly utilities, of VGAM261 correlate with, and may be deduced from, the identity of the target genes which VGAM261 binds and inhibits, and the function of these target genes, as elaborated hereinbelow.

JJAZ1 (Accession NM_{—}015355) is a VGAM261 host target gene. JJAZ1 BINDING SITE is HOST TARGET binding site found in the 3′ untranslated region of mRNA encoded by JJAZ1, corresponding to a HOST TARGET binding site such as BINDING SITE I, BINDING SITE II or BINDING SITE III. Table 2 illustrates the complementarity of the nucleotide sequences of JJAZ1 BINDING SITE, designated SEQ ID:1621, to the nucleotide sequence of VGAM261 RNA, herein designated VGAM RNA, also designated SEQ ID:596.

A function of VGAM261 is therefore inhibition of JJAZ1 (Accession NM_{—}015355), a gene which is a zinc finger protein. Accordingly, utilities of VGAM261 include diagnosis, prevention and treatment of diseases and clinical conditions associated with JJAZ1. The function of JJAZ1 and its association with various diseases and clinical conditions, has been established by previous studies, as described hereinabove with reference to VGAM187. Sialyltransferase 1 (beta-galactoside alpha-2,6-sialytransferase) (SIAT1, Accession NM_{—}003032) is another VGAM261 host target gene. SIAT1 BINDING SITE is HOST TARGET binding site found in the 3′ untranslated region of mRNA encoded by SIAT1, corresponding to a HOST TARGET binding site such as BINDING SITE I, BINDING SITE II or BINDING SITE III. Table 2 illustrates the complementarity of the nucleotide sequences of SIAT1 BINDING SITE, designated SEQ ID:986, to the nucleotide sequence of VGAM261 RNA, herein designated VGAM RNA, also designated SEQ ID:596.

Another function of VGAM261 is therefore inhibition of Sialyltransferase 1 (beta-galactoside alpha-2,6-sialytransferase) (SIAT1, Accession NM_{—}003032), a gene which transfers sialic acid from the donor of substrate cmp-sialic acid to galactose containing acceptor substrates. Accordingly, utilities of VGAM261 include diagnosis, prevention and treatment of diseases and clinical conditions associated with SIAT1. The function of SIAT1 has been established by previous studies. Much interest in the role and regulation of beta-galactoside alpha-2,6-sialyltransferase (EC 2.4.99.1) in B lymphocytes stemmed from its relation to CDw75, a human leukocyte cell-surface antigen expressed in mature and activated B cells but not in B cells at earlier stages of development or in plasma cells. SiaT-1 is required for the elaboration of the CDw75 cell-surface epitope. Grundmann et al. (1990) reported the complete cDNA sequence corresponding to the SIAT1 gene on the basis of cDNA isolated from a human placental lambda-gt10 library. By Southern analysis of somatic cell hybrids and by in situ hybridization, Wang et al. (1993) demonstrated that the SIAT1 gene is located on 3q21-q28. Comparative analysis of the human and rat sequences demonstrated precise conservation of the intron/exon boundaries throughout the coding domains. Furthermore, there was extensive interspecies sequence similarity in some of the exons that contained information only for the 5-prime leader regions.

Full details of the abovementioned studies are described in the following publications, the disclosure of which are hereby incorporated by reference:
Grundmann, U.; Nerlich, C.; Rein, T.; Zettlmeissl, G.: Complete cDNA sequence encoding human beta-galactoside alpha-2,6-sialyltransferase. Nucleic Acids Res. 18: 667 only, 1990.; and
Wang, X.; Vertino, A.; Eddy, R. L.; Byers, M. G.; Jani-Sait, S. N.; Shows, T. B.; Lau, J. T. Y.: Chromosome mapping and organization of the human beta-galactoside alpha-2,6-sialyltrans.

Further studies establishing the function and utilities of SIAT1 are found in John Hopkins OMIM database record ID 109675, and in sited publications numbered 869-870 listed in the bibliography section hereinbelow, which are also hereby incorporated by reference. ATPase, H+ Transporting, Lysosomal V0 Subunit A Isoform 1 (ATP6V0A1, Accession NM_{—}005177) is another VGAM261 host target gene. ATP6V0A1 BINDING SITE is HOST TARGET binding site found in the 3′ untranslated region of mRNA encoded by ATP6V0A1, corresponding to a HOST TARGET binding site such as BINDING SITE I, BINDING SITE II or BINDING SITE III. Table 2 illustrates the complementarity of the nucleotide sequences of ATP6V0A1 BINDING SITE, designated SEQ ID:1191, to the nucleotide sequence of VGAM261 RNA, herein designated VGAM RNA, also designated SEQ ID:596.

Another function of VGAM261 is therefore inhibition of ATPase, H+ Transporting, Lysosomal V0 Subunit A Isoform 1 (ATP6V0A1, Accession NM_{—}005177). Accordingly, utilities of VGAM261 include diagnosis, prevention and treatment of diseases and clinical conditions associated with ATP6V0A1. FLJ21313 (Accession NM_{—}023927) is another VGAM261 host target gene. FLJ21313 BINDING SITE is HOST TARGET binding site found in the 3′ untranslated region of mRNA encoded by FLJ21313, corresponding to a HOST TARGET binding site such as BINDING SITE I, BINDING SITE II or BINDING SITE III. Table 2 illustrates the complementarity of the nucleotide sequences of FLJ21313 BINDING SITE, designated SEQ ID:2038, to the nucleotide sequence of VGAM261 RNA, herein designated VGAM RNA, also designated SEQ ID:596.

Another function of VGAM261 is therefore inhibition of FLJ21313 (Accession NM_{—}023927). Accordingly, utilities of VGAM261 include diagnosis, prevention and treatment of diseases and clinical conditions associated with FLJ21313. FLJ23548 (Accession NM_{—}024590) is another VGAM261 host target gene. FLJ23548 BINDING SITE is HOST TARGET binding site found in the 3′ untranslated region of mRNA encoded by FLJ23548, corresponding to a HOST TARGET binding site such as BINDING SITE I, BINDING SITE II or BINDING SITE III. Table 2 illustrates the complementarity of the nucleotide sequences of FLJ23548 BINDING SITE, designated SEQ ID:2068, to the nucleotide sequence of VGAM261 RNA, herein designated VGAM RNA, also designated SEQ ID:596.

Another function of VGAM261 is therefore inhibition of FLJ23548 (Accession NM_{—}024590). Accordingly, utilities of VGAM261 include diagnosis, prevention and treatment of diseases and clinical conditions associated with FLJ23548. LOC196510 (Accession XM_{—}113738) is another VGAM261 host target gene. LOC196510 BINDING SITE is HOST TARGET binding site found in the 3′ untranslated region of mRNA encoded by LOC196510, corresponding to a HOST TARGET binding site such as BINDING SITE I, BINDING SITE II or BINDING SITE III. Table 2 illustrates the complementarity of the nucleotide sequences of LOC196510 BINDING SITE, designated SEQ ID:3414, to the nucleotide sequence of VGAM261 RNA, herein designated VGAM RNA, also designated SEQ ID:596.

Another function of VGAM261 is therefore inhibition of LOC196510 (Accession XM_{—}113738). Accordingly, utilities of VGAM261 include diagnosis, prevention and treatment of diseases and clinical conditions associated with LOC196510. LOC200220 (Accession XM_{—}114157) is another VGAM261 host target gene. LOC200220 BINDING SITE is HOST TARGET binding site found in the 3′ untranslated region of mRNA encoded by LOC200220, corresponding to a HOST TARGET binding site such as BINDING SITE I, BINDING SITE II or BINDING SITE III. Table 2 illustrates the complementarity of the nucleotide sequences of LOC200220 BINDING SITE, designated SEQ ID:3443, to the nucleotide sequence of VGAM261 RNA, herein designated VGAM RNA, also designated SEQ ID:596.

Another function of VGAM261 is therefore inhibition of LOC200220 (Accession XM_{—}114157). Accordingly, utilities of VGAM261 include diagnosis, prevention and treatment of diseases and clinical conditions associated with LOC200220. LOC222234 (Accession XM_{—}168558) is another VGAM261 host target gene. LOC222234 BINDING SITE is HOST TARGET binding site found in the 3′ untranslated region of mRNA encoded by LOC222234, corresponding to a HOST TARGET binding site such as BINDING SITE I, BINDING SITE II or BINDING SITE III. Table 2 illustrates the complementarity of the nucleotide sequences of LOC222234 BINDING SITE, designated SEQ ID:3645, to the nucleotide sequence of VGAM261 RNA, herein designated VGAM RNA, also designated SEQ ID:596.

Another function of VGAM261 is therefore inhibition of LOC222234 (Accession XM_{—}168558). Accordingly, utilities of VGAM261 include diagnosis, prevention and treatment of diseases and clinical conditions associated with LOC222234. FIG. 1 further provides a conceptual description of a novel bioinformatically detected viral gene of the present invention, referred to here as Viral Genomic Address Messenger 262 (VGAM262) viral gene, which modulates expression of respective host target genes thereof, the function and utility of which host target genes is known in the art.

VGAM262 is a novel bioinformatically detected regulatory, non protein coding, viral micro RNA (miRNA) gene. The method by which VGAM262 was detected is described hereinabove with reference to FIGS. 1-8.

VGAM262 gene, herein designated VGAM GENE, is a viral gene contained in the genome of Vaccinia Virus. VGAM262 host target gene, herein designated VGAM HOST TARGET GENE, is a human gene contained in the human genome.

VGAM262 gene encodes a VGAM262 precursor RNA, herein designated VGAM PRECURSOR RNA. Similar to other miRNA genes, and unlike most ordinary genes, VGAM262 precursor RNA does not encode a protein. A nucleotide sequence identical or highly similar to the nucleotide sequence of VGAM262 precursor RNA is designated SEQ ID:248, and is provided hereinbelow with reference to the sequence listing part. Nucleotide sequence SEQ ID:248 is located at position 147193 relative to the genome of Vaccinia Virus.

VGAM262 precursor RNA folds onto itself, forming VGAM262 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM262 folded precursor RNA into VGAM262 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM262 RNA is designated SEQ ID:597, and is provided hereinbelow with reference to the sequence listing part.

VGAM262 host target gene, herein designated VGAM HOST TARGET GENE, encodes a corresponding messenger RNA, VGAM262 host target RNA, herein designated VGAM HOST TARGET RNA. VGAM262 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM262 RNA, herein designated VGAM RNA, binds complementarily to one or more host target binding sites located in untranslated regions of VGAM262 host target RNA, herein designated VGAM HOST TARGET RNA. This complementary binding is due to the fact that the nucleotide sequence of VGAM262 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM262 RNA, herein designated VGAM RNA, may have a different number of host target binding sites in untranslated regions of a VGAM262 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM262 RNA, herein designated VGAM RNA, to host target binding sites on VGAM262 host target RNA, herein designated VGAM HOST TARGET RNA, such as BINDING SITE I, BINDING SITE II and BINDING SITE III, inhibits translation of VGAM262 host target RNA into VGAM262 host target protein, herein designated VGAM HOST TARGET PROTEIN. VGAM host target protein is therefore outlined by a broken line.

It is appreciated that VGAM262 host target gene, herein designated VGAM HOST TARGET GENE, in fact represents a plurality of VGAM262 host target genes. The mRNA of each one of this plurality of VGAM262 host target genes comprises one or more host target binding sites, each having a nucleotide sequence which is at least partly complementary to VGAM262 RNA, herein designated VGAM RNA, and which when bound by VGAM262 RNA causes inhibition of translation of respective one or more VGAM262 host target proteins.

It is further appreciated by one skilled in the art that the mode of translational inhibition illustrated by FIG. 1 with specific reference to translational inhibition exerted by VGAM262 gene, herein designated VGAM GENE, on one or more VGAM262 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM262 is inhibition of expression of host target genes, as part of a novel viral mechanism of attacking a host. Accordingly, utilities of VGAM262 include diagnosis, prevention and treatment of viral infection by Vaccinia Virus. Specific functions, and accordingly utilities, of VGAM262 correlate with, and may be deduced from, the identity of the host target genes which VGAM262 binds and inhibits, and the function of these host target genes, as elaborated hereinbelow.

Nucleotide sequences of the VGAM262 precursor RNA, herein designated VGAM PRECURSOR RNA, and of the ‘diced’ VGAM262 RNA, herein designated VGAM RNA, and a schematic representation of the secondary folding of VGAM262 folded precursor RNA, herein designated VGAM FOLDED PRECURSOR RNA, of VGAM262 are further described hereinbelow with reference to Table 1.

Nucleotide sequences of host target binding sites, such as BINDING SITE-I, BINDING SITE-II and BINDING SITE-III of FIG. 1, found on VGAM262 host target RNA, and schematic representation of the complementarity of each of these host target binding sites to VGAM262 RNA, herein designated VGAM RNA, are described hereinbelow with reference to Table 2.

As mentioned hereinabove with reference to FIG. 1, a function of VGAM262 gene, herein designated VGAM is inhibition of expression of VGAM262 target genes. It is appreciated that specific functions, and accordingly utilities, of VGAM262 correlate with, and may be deduced from, the identity of the target genes which VGAM262 binds and inhibits, and the function of these target genes, as elaborated hereinbelow.

Cyclin-dependent Kinase Inhibitor 1B (p27, Kip1) (CDKN1B, Accession NM_{—}004064) is a VGAM262 host target gene. CDKN1B BINDING SITE is HOST TARGET binding site found in the 3′ untranslated region of mRNA encoded by CDKN1B, corresponding to a HOST TARGET binding site such as BINDING SITE I, BINDING SITE II or BINDING SITE III. Table 2 illustrates the complementarity of the nucleotide sequences of CDKN1B BINDING SITE, designated SEQ ID: 1082, to the nucleotide sequence of VGAM262 RNA, herein designated VGAM RNA, also designated SEQ ID:597.

A function of VGAM262 is therefore inhibition of Cyclin-dependent Kinase Inhibitor 1B (p27, Kip1) (CDKN1B, Accession NM_{—}004064), a gene which is involved in g1 arrest and may mediate tgf beta-induced g1 arrest. Accordingly, utilities of VGAM262 include diagnosis, prevention and treatment of diseases and clinical conditions associated with CDKN1B. The function of CDKN1B has been established by previous studies. Stegmaier et al. (1995) studied loss of heterozygosity (LOH) in the region 12p13-p12 in acute lymphoblastic leukemia; this chromosomal region often shows deletion in such patients. In 15% of informative patients, there was evidence of LOH of the TEL locus (OMIM Ref. No. 600618) which was not evident on cytogenetic analysis. Detailed examination of patients with LOH showed that the critically deleted region included a second candidate tumor suppressor gene, referred to by them as KIP1, which encodes the cyclin-dependent kinase inhibitor previously called p27 (Toyoshima and Hunter, 1994 and Polyak et al., 1994). Based on the STS content of TEL-positive YACs, Stegmaier et al. (1995) reported that KIP1 and TEL were in close proximity. Apoptosis of human endothelial cells after growth factor deprivation is associated with rapid and dramatic up-regulation of cyclin A-associated CDK2 activity. Levkau et al. (1998) showed that in apoptotic cells the carboxyl-termini of the CDK inhibitors CDKN1A (OMIM Ref. No. 116899) and CDKN1B are truncated by specific cleavage. The enzyme involved in this cleavage is CASP3 (OMIM Ref. No. 600636) and/or a CASP3-like caspase. After cleavage, CDKN1A loses its nuclear localization sequence and exits the nucleus. Cleavage of CDKN1A and CDKN1B resulted in a substantial reduction in their association with nuclear cyclin-CDK2 complexes, leading to a dramatic induction of CDK2 activity. Dominant-negative CDK2, as well as a mutant CDKN1A resistant to caspase cleavage, partially suppressed apoptosis. These data suggested that CDK2 activation, through caspase-mediated cleavage of CDK inhibitors, may be instrumental in the execution of apoptosis following caspase activation.

Full details of the abovementioned studies are described in the following publications, the disclosure of which are hereby incorporated by reference:
Levkau, B.; Koyama, H.; Raines, E. W.; Clurman, B. E.; Herren, B.; Orth, K.; Roberts, J. M.; Ross, R.: Cleavage of p21(Cip1/Waf1) and p27(Kip1) mediates apoptosis in endothelial cells through activation of Cdk2: role of a caspase cascade. Molec. Cell 1: 553-563, 1998.; and
Stegmaier, K.; Pendse, S.; Barker, G. F.; Bray-Ward, P.; Ward, D. C.; Montgomery, K. T.; Krauter, K. S.; Reynolds, C.; Sklar, J.; Donnelly, M.; Bohlander, S. K.; Rowley, J. D.; Sallan.

Further studies establishing the function and utilities of CDKN1B are found in John Hopkins OMIM database record ID 600778, and in sited publications numbered 1318, 2223-2226, 1629, 2232-2233, 2860, 2235-2236, 1056, 223 and 2238 listed in the bibliography section hereinbelow, which are also hereby incorporated by reference. Chondroitin Sulfate Proteoglycan 3 (neurocan) (CSPG3, Accession NM_{—}004386) is another VGAM262 host target gene. CSPG3 BINDING SITE is HOST TARGET binding site found in the 3′ untranslated region of mRNA encoded by CSPG3, corresponding to a HOST TARGET binding site such as BINDING SITE I, BINDING SITE II or BINDING SITE III. Table 2 illustrates the complementarity of the nucleotide sequences of CSPG3 BINDING SITE, designated SEQ ID:1107, to the nucleotide sequence of VGAM262 RNA, herein designated VGAM RNA, also designated SEQ ID:597.

Another function of VGAM262 is therefore inhibition of Chondroitin Sulfate Proteoglycan 3 (neurocan) (CSPG3, Accession NM_{—}004386), a gene which may play a role in modulating cell adhesion and migration. Accordingly, utilities of VGAM262 include diagnosis, prevention and treatment of diseases and clinical conditions associated with CSPG3. The function of CSPG3 has been established by previous studies. Neurocan was first described in the early postnatal rat brain where it accounts for 20 to 30% of the total chondroitin sulfate proteoglycan. Rauch et al. (1992) cloned the rat cDNA using degenerate primers based on partial amino acid sequence of immunoaffinity-purified protein. The mouse neurocan cDNA encodes a deduced 1,257-amino acid protein with a predicted molecular mass of 136 kD. The large protein is processed into a smaller form in the adult brain. The predicted protein has a 22-amino acid signal peptide followed by an immunoglobin-like domain and repeating motifs characteristic of the hyaluronic acid-binding region of aggregating proteoglycans. The C terminus shows approximately 60% identity to the fibroblast and cartilage proteoglycans versican (OMIM Ref. No. 118661) and aggrecan (OMIM Ref. No. 155760). Northern blots detected a 7.5-kb transcript from 4-day and adult rat brains Prange et al. (1998) cloned human neurocan cDNAs from infant and adult brain cDNA libraries. The deduced 1,321-amino acid protein shares 63% sequence identity with both mouse and rat neurocan proteins. Like other known proteoglycans, its N terminus contains an immunoglobulin domain and a series of hyaluronic acid-binding tandem repeats, and its C terminus contains an EGF-like domain, a lectin-like domain, and a complement regulatory-like domain. Northern blot analysis detected expression of a 7.5-kb transcript in fetal and adult tissues from all brain regions tested

Full details of the abovementioned studies are described in the following publications, the disclosure of which are hereby incorporated by reference:
Rauch, U.; Grimpe, B.; Kulbe, G.; Arnold-Ammer, I.; Beier, D. R.; Fassler, R.: Structure and chromosomal localization of the mouse neurocan gene. Genomics 28: 405-410, 1995.; and
Prange, C. K.; Pennacchio, L. A.; Lieuallen, K.; Fan, W.; Lennon, G. G.: Characterization of the human neurocan gene, CSPG3. Gene 221: 199-205, 1998.

Further studies establishing the function and utilities of CSPG3 are found in John Hopkins OMIM database record ID 600826, and in sited publications numbered 1779-1782 listed in the bibliography section hereinbelow, which are also hereby incorporated by reference. Intersectin 1 (SH3 domain protein) (ITSN1, Accession NM_{—}003024) is another VGAM262 host target gene. ITSN1 BINDING SITE is HOST TARGET binding site found in the 3′ untranslated region of mRNA encoded by ITSN1, corresponding to a HOST TARGET binding site such as BINDING SITE I, BINDING SITE II or BINDING SITE III. Table 2 illustrates the complementarity of the nucleotide sequences of ITSN1 BINDING SITE, designated SEQ ID:981, to the nucleotide sequence of VGAM262 RNA, herein designated VGAM RNA, also designated SEQ ID:597.

Another function of VGAM262 is therefore inhibition of Intersectin 1 (SH3 domain protein) (ITSN1, Accession NM_{—}003024), a gene which may be involved in endocytosis and synaptic vesicle recycling. Accordingly, utilities of VGAM262 include diagnosis, prevention and treatment of diseases and clinical conditions associated with ITSN1. The function of ITSN1 has been established by previous studies. See 147265. Ozcelik et al. (1991) found that a cDNA probe for ITPR3 hybridized to DNA from hybrid cells containing human chromosome 6. In one hybrid that carried 6pter-p21, in the absence of an intact copy of this chromosome, hybridization was observed, thus mapping the gene to 6pter-p21. ITPR3 transduces many hormonal signals that regulate Ca(2+)-dependent processes in the intestinal epithelium. Maranto (1994) described complete sequence of the ITPR3 polypeptide (2,671 amino acids). Primary structure analysis indicated a pattern of conserved and variable regions, characteristic of the particular gene family. Immunocytochemical localization in the intestine was determined. Yamamoto-Hino et al. (1994) likewise mapped the ITPR3 gene to chromosome 6, specifically to 6p21, by isotopic in situ hybridization. They showed that the type 3 receptor was present in all hematopoietic and lymphoma cell lines tested

Full details of the abovementioned studies are described in the following publications, the disclosure of which are hereby incorporated by reference:
Maranto, A. R.: Primary structure, ligand binding, and localization of the human type 3 inositol 1,4,5-trisphosphate receptor expressed in intestinal epithelium. J. Biol. Chem. 269: 1222-1230, 1994.; and
Ozcelik, T.; Suedhof, T. C.; Francke, U.: The genes for inositol 1,4,5-triphosphate receptors 1 (ITPR1) and 3 (ITPR3) are localized on human chromosomes 3p and 6pter-p21, respectively.

Further studies establishing the function and utilities of ITSN1 are found in John Hopkins OMIM database record ID 602442, and in sited publications numbered 1295-1296, 134 and 1351 listed in the bibliography section hereinbelow, which are also hereby incorporated by reference. Muscleblind-like (Drosophila) (MBNL, Accession NM_{—}021038) is another VGAM262 host target gene. MBNL BINDING SITE is HOST TARGET binding site found in the 5′ untranslated region of mRNA encoded by MBNL, corresponding to a HOST TARGET binding site such as BINDING SITE I, BINDING SITE II or BINDING SITE III. Table 2 illustrates the complementarity of the nucleotide sequences of MBNL BINDING SITE, designated SEQ ID:1937, to the nucleotide sequence of VGAM262 RNA, herein designated VGAM RNA, also designated SEQ ID:597.

Another function of VGAM262 is therefore inhibition of Muscleblind-like (Drosophila) (MBNL, Accession NM_{—}021038), a gene which binds to cug triplet repeat expansion dsrna (by similarity). Accordingly, utilities of VGAM262 include diagnosis, prevention and treatment of diseases and clinical conditions associated with MBNL. The function of MBNL and its association with various diseases and clinical conditions, has been established by previous studies, as described hereinabove with reference to VGAM196. Mediterranean Fever (MEFV, Accession NM_{—}000243) is another VGAM262 host target gene. MEFV BINDING SITE is HOST TARGET binding site found in the 3′ untranslated region of mRNA encoded by MEFV, corresponding to a HOST TARGET binding site such as BINDING SITE I, BINDING SITE II or BINDING SITE III. Table 2 illustrates the complementarity of the nucleotide sequences of MEFV BINDING SITE, designated SEQ ID:719, to the nucleotide sequence of VGAM262 RNA, herein designated VGAM RNA, also designated SEQ ID:597.

Another function of VGAM262 is therefore inhibition of Mediterranean Fever (MEFV, Accession NM_{—}000243). Accordingly, utilities of VGAM262 include diagnosis, prevention and treatment of diseases and clinical conditions associated with MEFV. Nuclear Receptor Sub family 3, Group C, Member 1 (glucocorticoid receptor) (NR3C1, Accession NM_{—}000176) is another VGAM262 host target gene. NR3C1 BINDING SITE is HOST TARGET binding site found in the 3′ untranslated region of mRNA encoded by NR3C1, corresponding to a HOST TARGET binding site such as BINDING SITE I, BINDING SITE II or BINDING SITE III. Table 2 illustrates the complementarity of the nucleotide sequences of NR3C1 BINDING SITE, designated SEQ ID:716, to the nucleotide sequence of VGAM262 RNA, herein designated VGAM RNA, also designated SEQ ID:597.

Another function of VGAM262 is therefore inhibition of Nuclear Receptor Sub family 3, Group C, Member 1 (glucocorticoid receptor) (NR3C1, Accession NM_{—}000176). Accordingly, utilities of VGAM262 include diagnosis, prevention and treatment of diseases and clinical conditions associated with NR3C1. Transcriptional Intermediary Factor 1 (TIF1, Accession XM_{—}016701) is another VGAM262 host target gene. TIF1 BINDING SITE is HOST TARGET binding site found in the 3′ untranslated region of mRNA encoded by TIF1, corresponding to a HOST TARGET binding site such as BINDING SITE I, BINDING SITE II or BINDING SITE III. Table 2 illustrates the complementarity of the nucleotide sequences of TIF1 BINDING SITE, designated SEQ ID:2564, to the nucleotide sequence of VGAM262 RNA, herein designated VGAM RNA, also designated SEQ ID:597.

Another function of VGAM262 is therefore inhibition of Transcriptional Intermediary Factor 1 (TIF1, Accession XM_{—}016701), a gene which mediates the activation function (AF-2) of nuclear estrogen receptor. Accordingly, utilities of VGAM262 include diagnosis, prevention and treatment of diseases and clinical conditions associated with TIF1. The function of TIF1 has been established by previous studies. Hormonal regulation of gene activity is mediated by nuclear receptors acting as ligand-activated transcription factors. The activity of the ligand-dependent activation function, or AF2, of the receptors requires intermediary factors that interact with the AF2-activating domain, a C-terminal region that is highly conserved in the nuclear receptor family. Thenot et al. (1997) isolated human breast cancer cell cDNAs that encode transcription intermediary factor-1 (TIF1), a protein that is able to bind to the AF2-activating domain of the estrogen receptor (ESR; e.g., 133430). The deduced 1,013-amino acid TIF1 protein, which is more than 92% conserved with mouse Tif1, contains several domains: a RING finger, B-box fingers, a coiled-coil domain, a PHD homeodomain finger, and a bromodomain. A 26-amino acid region of TIF1 is sufficient for its hormone-dependent binding to the ESR. Thenot et al. (1997) demonstrated that the AF2-activating domain of ESR is required but not sufficient for the binding of TIF1, that TIF1 association with DNA-bound ESR requires the presence of estradiol, and that TIF1 interacts selectively with different nuclear receptors. The authors identified a cDNA variant that encodes a TIF1 isoform containing a 34-amino acid insertion. Northern blot analysis detected a major 4.5-kb transcript in MCF7 breast cancer cells. Fusion of PML (OMIM Ref. No. 102578) and TIF1A to RARA (OMIM Ref. No. 180240) and BRAF (OMIM Ref. No. 164757), respectively, results in the production of PML-RAR-alpha and TIF1-alpha-B-RAF (T18) oncoproteins. Zhong et al. (1999) showed that PML, TIF1-alpha, and RXR-alpha (OMIM Ref. No. 180245)/RAR-alpha function together in a retinoic acid-dependent transcription complex. Zhong et al. (1999) found that PML acts as a ligand-dependent coactivator of RXR-alpha/RARA-alpha. PML interacts with TIF1-alpha and CREB-binding protein (CBP; 600140). In PML −/− cells, the retinoic acid-dependent induction of genes such as RARB2 and the ability of TIF1-alpha and CBP to act as transcriptional coactivators on retinoic acid are impaired. Zhong et al. (1999) showed that both PML and TIF1-alpha are growth suppressors required for the growth-inhibitory activity of retinoic acid. T18, similar to PML-RAR-alpha, disrupts the retinoic acid-dependent activity of this complex in a dominant-negative manner, resulting in a growth advantage. PML-RAR-alpha was the first example of an oncoprotein generated by the fusion of 2 molecules participating in the same pathway, specifically the fusion of a transcription factor to one of its own cofactors. Since the PML and RAR-alpha pathways converge at the transcriptional level, there is no need for a double-dominant-negative product to explain the pathogenesis of acute promyelocytic leukemia, or APL. Beckstead et al. (2001) found that the Drosophila ‘bonus’ (bon) gene encodes a homolog of the vertebrate TIF1 transcriptional cofactors. Bon is required for male viability, molting, and numerous events in metamorphosis, including leg elongation, bristle development, and pigmentation. Most of these processes are associated with genes that are implicated in the ecdysone pathway, a nuclear hormone receptor pathway required throughout Drosophila development. Bon is associated with sites on the polytene chromosomes and can interact with numerous Drosophila nuclear receptor proteins. In vivo, bon behaves as a transcriptional inhibitor.

Full details of the abovementioned studies are described in the following publications, the disclosure of which are hereby incorporated by reference:
Zhong, S.; Delva, L.; Rachez, C.; Cenciarelli, C.; Gandini, D.; Zhang, H.; Kalantry, S.; Freedman, L. P.; Pandolfi, P. P.: A RA-dependent, tumour-growth suppressive transcription complex is the target of the PML-RAR-alpha and T18 oncoproteins. Nature Genet. 23: 287-295, 1999.; and
Beckstead, R.; Ortiz, J. A.; Sanchez, C.; Prokopenko, S. N.; Chambon, P.; Losson, R.; Bellen, H. J.: Bonus, a Drosophila homolog of TIF1 proteins, interacts with nuclear receptors and.

Further studies establishing the function and utilities of TIF1 are found in John Hopkins OMIM database record ID 603406, and in sited publications numbered 1202, 1203-1204, 144 and 2606 listed in the bibliography section hereinbelow, which are also hereby incorporated by reference. Basic Leucine Zipper and W2 Domains 1 (BZW1, Accession NM_{—}014670) is another VGAM262 host target gene. BZW1 BINDING SITE is HOST TARGET binding site found in the 3′ untranslated region of mRNA encoded by BZW1, corresponding to a HOST TARGET binding site such as BINDING SITE I, BINDING SITE II or BINDING SITE III. Table 2 illustrates the complementarity of the nucleotide sequences of BZW1 BINDING SITE, designated SEQ ID:1516, to the nucleotide sequence of VGAM262 RNA, herein designated VGAM RNA, also designated SEQ ID:597.

Another function of VGAM262 is therefore inhibition of Basic Leucine Zipper and W2 Domains 1 (BZW1, Accession NM_{—}014670). Accordingly, utilities of VGAM262 include diagnosis, prevention and treatment of diseases and clinical conditions associated with BZW1. FLJ10998 (Accession NM_{—}018294) is another VGAM262 host target gene. FLJ10998 BINDING SITE is HOST TARGET binding site found in the 3′ untranslated region of mRNA encoded by FLJ10998, corresponding to a HOST TARGET binding site such as BINDING SITE I, BINDING SITE II or BINDING SITE III. Table 2 illustrates the complementarity of the nucleotide sequences of FLJ10998 BINDING SITE, designated SEQ ID:1811, to the nucleotide sequence of VGAM262 RNA, herein designated VGAM RNA, also designated SEQ ID:597.

Another function of VGAM262 is therefore inhibition of FLJ10998 (Accession NM_{—}018294). Accordingly, utilities of VGAM262 include diagnosis, prevention and treatment of diseases and clinical conditions associated with FLJ10998. FLJ12409 (Accession NM_{—}025105) is another VGAM262 host target gene. FLJ12409 BINDING SITE is HOST TARGET binding site found in the 3′ untranslated region of mRNA encoded by FLJ12409, corresponding to a HOST TARGET binding site such as BINDING SITE I, BINDING SITE II or BINDING SITE III. Table 2 illustrates the complementarity of the nucleotide sequences of FLJ12409 BINDING SITE, designated SEQ ID:2137, to the nucleotide sequence of VGAM262 RNA, herein designated VGAM RNA, also designated SEQ ID:597.

Another function of VGAM262 is therefore inhibition of FLJ12409 (Accession NM_{—}025105). Accordingly, utilities of VGAM262 include diagnosis, prevention and treatment of diseases and clinical conditions associated with FLJ12409. FLJ32356 (Accession NM_{—}144671) is another VGAM262 host target gene. FLJ32356 BINDING SITE is HOST TARGET binding site found in the 3′ untranslated region of mRNA encoded by FLJ32356, corresponding to a HOST TARGET binding site such as BINDING SITE I, BINDING SITE II or BINDING SITE III. Table 2 illustrates the complementarity of the nucleotide sequences of FLJ32356 BINDING SITE, designated SEQ ID:2492, to the nucleotide sequence of VGAM262 RNA, herein designated VGAM RNA, also designated SEQ ID:597.

Another function of VGAM262 is therefore inhibition of FLJ32356 (Accession NM_{—}144671). Accordingly, utilities of VGAM262 include diagnosis, prevention and treatment of diseases and clinical conditions associated with FLJ32356. GRP3 (Accession NM_{—}015376) is another VGAM262 host target gene. GRP3 BINDING SITE is HOST TARGET binding site found in the 3′ untranslated region of mRNA encoded by GRP3, corresponding to a HOST TARGET binding site such as BINDING SITE I, BINDING SITE II or BINDING SITE III. Table 2 illustrates the complementarity of the nucleotide sequences of GRP3 BINDING SITE, designated SEQ ID: 1624, to the nucleotide sequence of VGAM262 RNA, herein designated VGAM RNA, also designated SEQ ID:597.

Another function of VGAM262 is therefore inhibition of GRP3 (Accession NM_{—}015376). Accordingly, utilities of VGAM262 include diagnosis, prevention and treatment of diseases and clinical conditions associated with GRP3. KIAA0210 (Accession NM_{—}014744) is another VGAM262 host target gene. KIAA0210 BINDING SITE is HOST TARGET binding site found in the 3′ untranslated region of mRNA encoded by KIAA0210, corresponding to a HOST TARGET binding site such as BINDING SITE I, BINDING SITE II or BINDING SITE III. Table 2 illustrates the complementarity of the nucleotide sequences of KIAA0210 BINDING SITE, designated SEQ ID:1538, to the nucleotide sequence of VGAM262 RNA, herein designated VGAM RNA, also designated SEQ ID:597.

Another function of VGAM262 is therefore inhibition of KIAA0210 (Accession NM_{—}014744). Accordingly, utilities of VGAM262 include diagnosis, prevention and treatment of diseases and clinical conditions associated with KIAA0210. MGC2477 (Accession NM_{—}024099) is another VGAM262 host target gene. MGC2477 BINDING SITE is HOST TARGET binding site found in the 3′ untranslated region of mRNA encoded by MGC2477, corresponding to a HOST TARGET binding site such as BINDING SITE I, BINDING SITE II or BINDING SITE III. Table 2 illustrates the complementarity of the nucleotide sequences of MGC2477 BINDING SITE, designated SEQ ID:2054, to the nucleotide sequence of VGAM262 RNA, herein designated VGAM RNA, also designated SEQ ID:597.

Another function of VGAM262 is therefore inhibition of MGC2477 (Accession NM_{—}024099). Accordingly, utilities of VGAM262 include diagnosis, prevention and treatment of diseases and clinical conditions associated with MGC2477. MGC2742 (Accession NM_{—}023938) is another VGAM262 host target gene. MGC2742 BINDING SITE is HOST TARGET binding site found in the 5′ untranslated region of mRNA encoded by MGC2742, corresponding to a HOST TARGET binding site such as BINDING SITE I, BINDING SITE II or BINDING SITE III. Table 2 illustrates the complementarity of the nucleotide sequences of MGC2742 BINDING SITE, designated SEQ ID:2039, to the nucleotide sequence of VGAM262 RNA, herein designated VGAM RNA, also designated SEQ ID:597.

Another function of VGAM262 is therefore inhibition of MGC2742 (Accession NM_{—}023938). Accordingly, utilities of VGAM262 include diagnosis, prevention and treatment of diseases and clinical conditions associated with MGC2742. MRF2 (Accession XM_{—}084482) is another VGAM262 host target gene. MRF2 BINDING SITE is HOST TARGET binding site found in the 3′ untranslated region of mRNA encoded by MRF2, corresponding to a HOST TARGET binding site such as BINDING SITE I, BINDING SITE II or BINDING SITE III. Table 2 illustrates the complementarity of the nucleotide sequences of MRF2 BINDING SITE, designated SEQ ID:3056, to the nucleotide sequence of VGAM262 RNA, herein designated VGAM RNA, also designated SEQ ID:597.

Another function of VGAM262 is therefore inhibition of MRF2 (Accession XM_{—}084482). Accordingly, utilities of VGAM262 include diagnosis, prevention and treatment of diseases and clinical conditions associated with MRF2. Purinergic Receptor P2X-like 1, Orphan Receptor (P2RXL1, Accession NM_{—}005446) is another VGAM262 host target gene. P2RXL1 BINDING SITE is HOST TARGET binding site found in the 3′ untranslated region of mRNA encoded by P2RXL1, corresponding to a HOST TARGET binding site such as BINDING SITE I, BINDING SITE II or BINDING SITE III. Table 2 illustrates the complementarity of the nucleotide sequences of P2RXL1 BINDING SITE, designated SEQ ID:1213, to the nucleotide sequence of VGAM262 RNA, herein designated VGAM RNA, also designated SEQ ID:597.

Another function of VGAM262 is therefore inhibition of Purinergic Receptor P2X-like 1, Orphan Receptor (P2RXL1, Accession NM_{—}005446). Accordingly, utilities of VGAM262 include diagnosis, prevention and treatment of diseases and clinical conditions associated with P2RXL1. Serine/threonine Kinase 38 Like (STK38L, Accession XM_{—}044823) is another VGAM262 host target gene. STK38L BINDING SITE is HOST TARGET binding site found in the 3′ untranslated region of mRNA encoded by STK38L, corresponding to a HOST TARGET binding site such as BINDING SITE I, BINDING SITE II or BINDING SITE III. Table 2 illustrates the complementarity of the nucleotide sequences of STK38L BINDING SITE, designated SEQ ID:2844, to the nucleotide sequence of VGAM262 RNA, herein designated VGAM RNA, also designated SEQ ID:597.

Another function of VGAM262 is therefore inhibition of Serine/threonine Kinase 38 Like (STK38L, Accession XM_{—}044823). Accordingly, utilities of VGAM262 include diagnosis, prevention and treatment of diseases and clinical conditions associated with STK38L. LOC151579 (Accession XM_{—}045290) is another VGAM262 host target gene. LOC151579 BINDING SITE is HOST TARGET binding site found in the 3′ untranslated region of mRNA encoded by LOC151579, corresponding to a HOST TARGET binding site such as BINDING SITE I, BINDING SITE II or BINDING SITE III. Table 2 illustrates the complementarity of the nucleotide sequences of LOC151579 BINDING SITE, designated SEQ ID:2859, to the nucleotide sequence of VGAM262 RNA, herein designated VGAM RNA, also designated SEQ ID:597.

Another function of VGAM262 is therefore inhibition of LOC151579 (Accession XM_{—}045290). Accordingly, utilities of VGAM262 include diagnosis, prevention and treatment of diseases and clinical conditions associated with LOC151579. LOC51312 (Accession NM_{—}018579) is another VGAM262 host target gene. LOC51312 BINDING SITE is HOST TARGET binding site found in the 5′ untranslated region of mRNA encoded by LOC51312, corresponding to a HOST TARGET binding site such as BINDING SITE I, BINDING SITE II or BINDING SITE III. Table 2 illustrates the complementarity of the nucleotide sequences of LOC51312 BINDING SITE, designated SEQ ID:1849, to the nucleotide sequence of VGAM262 RNA, herein designated VGAM RNA, also designated SEQ ID:597.

Another function of VGAM262 is therefore inhibition of LOC51312 (Accession NM_{—}018579). Accordingly, utilities of VGAM262 include diagnosis, prevention and treatment of diseases and clinical conditions associated with LOC51312. LOC91574 (Accession XM_{—}039310) is another VGAM262 host target gene. LOC91574 BINDING SITE is HOST TARGET binding site found in the 3′ untranslated region of mRNA encoded by LOC91574, corresponding to a HOST TARGET binding site such as BINDING SITE I, BINDING SITE II or BINDING SITE III. Table 2 illustrates the complementarity of the nucleotide sequences of LOC91574 BINDING SITE, designated SEQ ID:2751, to the nucleotide sequence of VGAM262 RNA, herein designated VGAM RNA, also designated SEQ ID:597.

Another function of VGAM262 is therefore inhibition of LOC91574 (Accession XM_{—}039310). Accordingly, utilities of VGAM262 include diagnosis, prevention and treatment of diseases and clinical conditions associated with LOC91574. FIG. 1 further provides a conceptual description of a novel bioinformatically detected viral gene of the present invention, referred to here as Viral Genomic Address Messenger 263 (VGAM263) viral gene, which modulates expression of respective host target genes thereof, the function and utility of which host target genes is known in the art.

VGAM263 is a novel bioinformatically detected regulatory, non protein coding, viral micro RNA (miRNA) gene. The method by which VGAM263 was detected is described hereinabove with reference to FIGS. 1-8.

VGAM263 gene, herein designated VGAM GENE, is a viral gene contained in the genome of Vaccinia Virus. VGAM263 host target gene, herein designated VGAM HOST TARGET GENE, is a human gene contained in the human genome.

VGAM263 gene encodes a VGAM263 precursor RNA, herein designated VGAM PRECURSOR RNA. Similar to other miRNA genes, and unlike most ordinary genes, VGAM263 precursor RNA does not encode a protein. A nucleotide sequence identical or highly similar to the nucleotide sequence of VGAM263 precursor RNA is designated SEQ ID:249, and is provided hereinbelow with reference to the sequence listing part. Nucleotide sequence SEQ ID:249 is located at position 147919 relative to the genome of Vaccinia Virus.

VGAM263 precursor RNA folds onto itself, forming VGAM263 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM263 folded precursor RNA into VGAM263 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM263 RNA is designated SEQ ID:598, and is provided hereinbelow with reference to the sequence listing part.

VGAM263 host target gene, herein designated VGAM HOST TARGET GENE, encodes a corresponding messenger RNA, VGAM263 host target RNA, herein designated VGAM HOST TARGET RNA. VGAM263 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM263 RNA, herein designated VGAM RNA, binds complementarily to one or more host target binding sites located in untranslated regions of VGAM263 host target RNA, herein designated VGAM HOST TARGET RNA. This complementary binding is due to the fact that the nucleotide sequence of VGAM263 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM263 RNA, herein designated VGAM RNA, may have a different number of host target binding sites in untranslated regions of a VGAM263 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM263 RNA, herein designated VGAM RNA, to host target binding sites on VGAM263 host target RNA, herein designated VGAM HOST TARGET RNA, such as BINDING SITE I, BINDING SITE II and BINDING SITE III, inhibits translation of VGAM263 host target RNA into VGAM263 host target protein, herein designated VGAM HOST TARGET PROTEIN. VGAM host target protein is therefore outlined by a broken line.

It is appreciated that VGAM263 host target gene, herein designated VGAM HOST TARGET GENE, in fact represents a plurality of VGAM263 host target genes. The mRNA of each one of this plurality of VGAM263 host target genes comprises one or more host target binding sites, each having a nucleotide sequence which is at least partly complementary to VGAM263 RNA, herein designated VGAM RNA, and which when bound by VGAM263 RNA causes inhibition of translation of respective one or more VGAM263 host target proteins.

It is further appreciated by one skilled in the art that the mode of translational inhibition illustrated by FIG. 1 with specific reference to translational inhibition exerted by VGAM263 gene, herein designated VGAM GENE, on one or more VGAM263 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM263 is inhibition of expression of host target genes, as part of a novel viral mechanism of attacking a host. Accordingly, utilities of VGAM263 include diagnosis, prevention and treatment of viral infection by Vaccinia Virus. Specific functions, and accordingly utilities, of VGAM263 correlate with, and may be deduced from, the identity of the host target genes which VGAM263 binds and inhibits, and the function of these host target genes, as elaborated hereinbelow.

Nucleotide sequences of the VGAM263 precursor RNA, herein designated VGAM PRECURSOR RNA, and of the ‘diced’ VGAM263 RNA, herein designated VGAM RNA, and a schematic representation of the secondary folding of VGAM263 folded precursor RNA, herein designated VGAM FOLDED PRECURSOR RNA, of VGAM263 are further described hereinbelow with reference to Table 1.

Nucleotide sequences of host target binding sites, such as BINDING SITE-I, BINDING SITE-II and BINDING SITE-III of FIG. 1, found on VGAM263 host target RNA, and schematic representation of the complementarity of each of these host target binding sites to VGAM263 RNA, herein designated VGAM RNA, are described hereinbelow with reference to Table 2.

As mentioned hereinabove with reference to FIG. 1, a function of VGAM263 gene, herein designated VGAM is inhibition of expression of VGAM263 target genes. It is appreciated that specific functions, and accordingly utilities, of VGAM263 correlate with, and may be deduced from, the identity of the target genes which VGAM263 binds and inhibits, and the function of these target genes, as elaborated hereinbelow.

Mab-21-like 2 (C. elegans) (MAB21L2, Accession NM_{—}006439) is a VGAM263 host target gene. MAB21L2 BINDING SITE is HOST TARGET binding site found in the 5′ untranslated region of mRNA encoded by MAB21L2, corresponding to a HOST TARGET binding site such as BINDING SITE I, BINDING SITE II or BINDING SITE III. Table 2 illustrates the complementarity of the nucleotide sequences of MAB21L2 BINDING SITE, designated SEQ ID:1301, to the nucleotide sequence of VGAM263 RNA, herein designated VGAM RNA, also designated SEQ ID:598.

A function of VGAM263 is therefore inhibition of Mab-21-like 2 (C. elegans) (MAB21L2, Accession NM_{—}006439). Accordingly, utilities of VGAM263 include diagnosis, prevention and treatment of diseases and clinical conditions associated with MAB21L2. LOC220672 (Accession XM_{—}017177) is another VGAM263 host target gene. LOC220672 BINDING SITE is HOST TARGET binding site found in the 3′ untranslated region of mRNA encoded by LOC220672, corresponding to a HOST TARGET binding site such as BINDING SITE I, BINDING SITE II or BINDING SITE III. Table 2 illustrates the complementarity of the nucleotide sequences of LOC220672 BINDING SITE, designated SEQ ID:2565, to the nucleotide sequence of VGAM263 RNA, herein designated VGAM RNA, also designated SEQ ID:598.

Another function of VGAM263 is therefore inhibition of LOC220672 (Accession XM_{—}017177). Accordingly, utilities of VGAM263 include diagnosis, prevention and treatment of diseases and clinical conditions associated with LOC220672. FIG. 1 further provides a conceptual description of a novel bioinformatically detected viral gene of the present invention, referred to here as Viral Genomic Address Messenger 264 (VGAM264) viral gene, which modulates expression of respective host target genes thereof, the function and utility of which host target genes is known in the art.

VGAM264 is a novel bioinformatically detected regulatory, non protein coding, viral micro RNA (miRNA) gene. The method by which VGAM264 was detected is described hereinabove with reference to FIGS. 1-8.

VGAM264 gene, herein designated VGAM GENE, is a viral gene contained in the genome of Vaccinia Virus. VGAM264 host target gene, herein designated VGAM HOST TARGET GENE, is a human gene contained in the human genome.

VGAM264 gene encodes a VGAM264 precursor RNA, herein designated VGAM PRECURSOR RNA. Similar to other miRNA genes, and unlike most ordinary genes, VGAM264 precursor RNA does not encode a protein. A nucleotide sequence identical or highly similar to the nucleotide sequence of VGAM264 precursor RNA is designated SEQ ID:250, and is provided hereinbelow with reference to the sequence listing part. Nucleotide sequence SEQ ID:250 is located at position 148297 relative to the genome of Vaccinia Virus.

VGAM264 precursor RNA folds onto itself, forming VGAM264 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM264 folded precursor RNA into VGAM264 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM264 RNA is designated SEQ ID:599, and is provided hereinbelow with reference to the sequence listing part.

VGAM264 host target gene, herein designated VGAM HOST TARGET GENE, encodes a corresponding messenger RNA, VGAM264 host target RNA, herein designated VGAM HOST TARGET RNA. VGAM264 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM264 RNA, herein designated VGAM RNA, binds complementarily to one or more host target binding sites located in untranslated regions of VGAM264 host target RNA, herein designated VGAM HOST TARGET RNA. This complementary binding is due to the fact that the nucleotide sequence of VGAM264 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM264 RNA, herein designated VGAM RNA, may have a different number of host target binding sites in untranslated regions of a VGAM264 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM264 RNA, herein designated VGAM RNA, to host target binding sites on VGAM264 host target RNA, herein designated VGAM HOST TARGET RNA, such as BINDING SITE I, BINDING SITE II and BINDING SITE III, inhibits translation of VGAM264 host target RNA into VGAM264 host target protein, herein designated VGAM HOST TARGET PROTEIN. VGAM host target protein is therefore outlined by a broken line.

It is appreciated that VGAM264 host target gene, herein designated VGAM HOST TARGET GENE, in fact represents a plurality of VGAM264 host target genes. The mRNA of each one of this plurality of VGAM264 host target genes comprises one or more host target binding sites, each having a nucleotide sequence which is at least partly complementary to VGAM264 RNA, herein designated VGAM RNA, and which when bound by VGAM264 RNA causes inhibition of translation of respective one or more VGAM264 host target proteins.

It is further appreciated by one skilled in the art that the mode of translational inhibition illustrated by FIG. 1 with specific reference to translational inhibition exerted by VGAM264 gene, herein designated VGAM GENE, on one or more VGAM264 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM264 is inhibition of expression of host target genes, as part of a novel viral mechanism of attacking a host. Accordingly, utilities of VGAM264 include diagnosis, prevention and treatment of viral infection by Vaccinia Virus. Specific functions, and accordingly utilities, of VGAM264 correlate with, and may be deduced from, the identity of the host target genes which VGAM264 binds and inhibits, and the function of these host target genes, as elaborated hereinbelow.

Nucleotide sequences of the VGAM264 precursor RNA, herein designated VGAM PRECURSOR RNA, and of the ‘diced’ VGAM264 RNA, herein designated VGAM RNA, and a schematic representation of the secondary folding of VGAM264 folded precursor RNA, herein designated VGAM FOLDED PRECURSOR RNA, of VGAM264 are further described hereinbelow with reference to Table 1.

Nucleotide sequences of host target binding sites, such as BINDING SITE-I, BINDING SITE-II and BINDING SITE-III of FIG. 1, found on VGAM264 host target RNA, and schematic representation of the complementarity of each of these host target binding sites to VGAM264 RNA, herein designated VGAM RNA, are described hereinbelow with reference to Table 2.

As mentioned hereinabove with reference to FIG. 1, a function of VGAM264 gene, herein designated VGAM is inhibition of expression of VGAM264 target genes. It is appreciated that specific functions, and accordingly utilities, of VGAM264 correlate with, and may be deduced from, the identity of the target genes which VGAM264 binds and inhibits, and the function of these target genes, as elaborated hereinbelow.

Fibronectin Leucine Rich Transmembrane Protein 2 (FLRT2, Accession NM_{—}013231) is a VGAM264 host target gene. FLRT2 BINDING SITE is HOST TARGET binding site found in the 3′ untranslated region of mRNA encoded by FLRT2, corresponding to a HOST TARGET binding site such as BINDING SITE I, BINDING SITE II or BINDING SITE III. Table 2 illustrates the complementarity of the nucleotide sequences of FLRT2 BINDING SITE, designated SEQ ID:1437, to the nucleotide sequence of VGAM264 RNA, herein designated VGAM RNA, also designated SEQ ID:599.

A function of VGAM264 is therefore inhibition of Fibronectin Leucine Rich Transmembrane Protein 2 (FLRT2, Accession NM_{—}013231), a gene which may have a function in cell adhesion and/or receptor signaling. Accordingly, utilities of VGAM264 include diagnosis, prevention and treatment of diseases and clinical conditions associated with FLRT2. The function of FLRT2 has been established by previous studies. The FLRT family of proteins structurally resembles small leucine-rich proteoglycans found in the extracellular matrix. By screening human brain cDNAs for the potential to encode proteins that are at least 50 kD, Ishikawa et al. (1997) isolated a FLRT2 cDNA, which they called KIAA0405. The deduced 660-amino acid full-length FLRT2 protein shares 25% amino acid sequence identity with the precursor of the alpha chain of human platelet glycoprotein 1b (GP1BA; OMIM Ref. No. 231200) across 180 residues. By SDS-PAGE, in vitro transcribed/translated FLRT2 had an apparent molecular mass of approximately 75 kD. RT-PCR detected FLRT2 expression in a number of human tissues, with highest expression in ovary and relatively high expression in brain and pancreas. By searching a human EST database with portions of the FLRT1 protein (OMIM Ref. No. 604806) sequence, Lacy et al. (1999) identified ESTs encoding FLRT2. The full-length FLRT2 coding sequence encodes a predicted 660-amino acid protein containing a putative N-terminal signal sequence, 10 leucine-rich repeats (LRRs) flanked by N- and C-terminal cysteine-rich regions, a fibronectin-/collagen-like domain, a transmembrane domain, and an intracellular C-terminal tail. FLRT2 has 5 potential N-glycosylation sites in its extracellular region. FLRT2 shares 44% amino acid sequence identity with FLRT3 (OMIM Ref. No. 604808) and 41% identity with FLRT1. Recombinant FLRT2 expressed in SF9 insect cells and monkey COS-1 cells migrated as an 85-kD protein on SDS-polyacrylamide gels. The authors demonstrated that FLRT2 is glycosylated. Northern blot analysis of a variety of human adult tissues detected a 7.5-kb FLRT2 transcript that was expressed abundantly in pancreas and less abundantly in skeletal muscle, brain, and heart. Lacy et al. (1999) suggested that FLRT2 functions in cell adhesion and/or receptor signaling. By analysis of a radiation hybrid mapping panel, Ishikawa et al. (1997) mapped the FLRT2 gene to chromosome 14. Lacy et al. (1999) noted that a UniGene cluster corresponding to the FLRT2 gene has been mapped to 14q24-q32.

Full details of the abovementioned studies are described in the following publications, the disclosure of which are hereby incorporated by reference:
Ishikawa, K.; Nagase, T.; Nakajima, D.; Seki, N.; Ohira, M.; Miyajima, N.; Tanaka, A.; Kotani, H.; Nomura, N.; Ohara, O.: Prediction of the coding sequences of unidentified human genes. VIII. 78 new cDNA clones from brain which code for large proteins in vitro. DNA Res. 4: 307-313, 1997.; and
Lacy, S. E.; Bonnemann, C. G.; Buzney, E. A.; Kunkel, L. M.: Identification of FLRT1, FLRT2, and FLRT3: a novel family of transmembrane leucine-rich repeat proteins. Genomics 62: 417-4.

Further studies establishing the function and utilities of FLRT2 are found in John Hopkins OMIM database record ID 604807, and in sited publications numbered 25 and 968 listed in the bibliography section hereinbelow, which are also hereby incorporated by reference. KDEL (Lys-Asp-Glu-Leu) Endoplasmic Reticulum Protein Retention Receptor 2 (KDELR2, Accession NM_{—}006854) is another VGAM264 host target gene. KDELR2 BINDING SITE is HOST TARGET binding site found in the 3′ untranslated region of mRNA encoded by KDELR2, corresponding to a HOST TARGET binding site such as BINDING SITE I, BINDING SITE II or BINDING SITE III. Table 2 illustrates the complementarity of the nucleotide sequences of KDELR2 BINDING SITE, designated SEQ ID:1335, to the nucleotide sequence of VGAM264 RNA, herein designated VGAM RNA, also designated SEQ ID:599.

Another function of VGAM264 is therefore inhibition of KDEL (Lys-Asp-Glu-Leu) Endoplasmic Reticulum Protein Retention Receptor 2 (KDELR2, Accession NM_{—}006854). Accordingly, utilities of VGAM264 include diagnosis, prevention and treatment of diseases and clinical conditions associated with KDELR2. SMT3 Suppressor of Mif Two 3 Homolog 2 (yeast) (SMT3H2, Accession NM_{—}006937) is another VGAM264 host target gene. SMT3H2BINDING SITE is HOST TARGET binding site found in the 3′ untranslated region of mRNA encoded by SMT3H2, corresponding to a HOST TARGET binding site such as BINDING SITE I, BINDING SITE II or BINDING SITE III. Table 2 illustrates the complementarity of the nucleotide sequences of SMT3H2BINDING SITE, designated SEQ ID:1341, to the nucleotide sequence of VGAM264 RNA, herein designated VGAM RNA, also designated SEQ ID:599.

Another function of VGAM264 is therefore inhibition of SMT3 Suppressor of Mif Two 3 Homolog 2 (yeast) (SMT3H2, Accession NM_{—}006937). Accordingly, utilities of VGAM264 include diagnosis, prevention and treatment of diseases and clinical conditions associated with SMT3H2. LOC158997 (Accession XM_{—}088736) is another VGAM264 host target gene. LOC158997 BINDING SITE is HOST TARGET binding site found in the 3′ untranslated region of mRNA encoded by LOC158997, corresponding to a HOST TARGET binding site such as BINDING SITE I, BINDING SITE II or BINDING SITE III. Table 2 illustrates the complementarity of the nucleotide sequences of LOC158997 BINDING SITE, designated SEQ ID:3230, to the nucleotide sequence of VGAM264 RNA, herein designated VGAM RNA, also designated SEQ ID:599.

Another function of VGAM264 is therefore inhibition of LOC158997 (Accession XM_{—}088736). Accordingly, utilities of VGAM264 include diagnosis, prevention and treatment of diseases and clinical conditions associated with LOC158997. FIG. 1 further provides a conceptual description of a novel bioinformatically detected viral gene of the present invention, referred to here as Viral Genomic Address Messenger 265 (VGAM265) viral gene, which modulates expression of respective host target genes thereof, the function and utility of which host target genes is known in the art.

VGAM265 is a novel bioinformatically detected regulatory, non protein coding, viral micro RNA (miRNA) gene. The method by which VGAM265 was detected is described hereinabove with reference to FIGS. 1-8.

VGAM265 gene, herein designated VGAM GENE, is a viral gene contained in the genome of Vaccinia Virus. VGAM265 host target gene, herein designated VGAM HOST TARGET GENE, is a human gene contained in the human genome.

VGAM265 gene encodes a VGAM265 precursor RNA, herein designated VGAM PRECURSOR RNA. Similar to other miRNA genes, and unlike most ordinary genes, VGAM265 precursor RNA does not encode a protein. A nucleotide sequence identical or highly similar to the nucleotide sequence of VGAM265 precursor RNA is designated SEQ ID:251, and is provided hereinbelow with reference to the sequence listing part. Nucleotide sequence SEQ ID:251 is located at position 148003 relative to the genome of Vaccinia Virus.

VGAM265 precursor RNA folds onto itself, forming VGAM265 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM265 folded precursor RNA into VGAM265 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM265 RNA is designated SEQ ID:600, and is provided hereinbelow with reference to the sequence listing part.

VGAM265 host target gene, herein designated VGAM HOST TARGET GENE, encodes a corresponding messenger RNA, VGAM265 host target RNA, herein designated VGAM HOST TARGET RNA. VGAM265 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM265 RNA, herein designated VGAM RNA, binds complementarily to one or more host target binding sites located in untranslated regions of VGAM265 host target RNA, herein designated VGAM HOST TARGET RNA. This complementary binding is due to the fact that the nucleotide sequence of VGAM265 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM265 RNA, herein designated VGAM RNA, may have a different number of host target binding sites in untranslated regions of a VGAM265 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM265 RNA, herein designated VGAM RNA, to host target binding sites on VGAM265 host target RNA, herein designated VGAM HOST TARGET RNA, such as BINDING SITE I, BINDING SITE II and BINDING SITE III, inhibits translation of VGAM265 host target RNA into VGAM265 host target protein, herein designated VGAM HOST TARGET PROTEIN. VGAM host target protein is therefore outlined by a broken line.

It is appreciated that VGAM265 host target gene, herein designated VGAM HOST TARGET GENE, in fact represents a plurality of VGAM265 host target genes. The mRNA of each one of this plurality of VGAM265 host target genes comprises one or more host target binding sites, each having a nucleotide sequence which is at least partly complementary to VGAM265 RNA, herein designated VGAM RNA, and which when bound by VGAM265 RNA causes inhibition of translation of respective one or more VGAM265 host target proteins.

It is further appreciated by one skilled in the art that the mode of translational inhibition illustrated by FIG. 1 with specific reference to translational inhibition exerted by VGAM265 gene, herein designated VGAM GENE, on one or more VGAM265 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM265 is inhibition of expression of host target genes, as part of a novel viral mechanism of attacking a host. Accordingly, utilities of VGAM265 include diagnosis, prevention and treatment of viral infection by Vaccinia Virus. Specific functions, and accordingly utilities, of VGAM265 correlate with, and may be deduced from, the identity of the host target genes which VGAM265 binds and inhibits, and the function of these host target genes, as elaborated hereinbelow.

Nucleotide sequences of the VGAM265 precursor RNA, herein designated VGAM PRECURSOR RNA, and of the ‘diced’ VGAM265 RNA, herein designated VGAM RNA, and a schematic representation of the secondary folding of VGAM265 folded precursor RNA, herein designated VGAM FOLDED PRECURSOR RNA, of VGAM265 are further described hereinbelow with reference to Table 1.

Nucleotide sequences of host target binding sites, such as BINDING SITE-I, BINDING SITE-II and BINDING SITE-III of FIG. 1, found on VGAM265 host target RNA, and schematic representation of the complementarity of each of these host target binding sites to VGAM265 RNA, herein designated VGAM RNA, are described hereinbelow with reference to Table 2.

As mentioned hereinabove with reference to FIG. 1, a function of VGAM265 gene, herein designated VGAM is inhibition of expression of VGAM265 target genes. It is appreciated that specific functions, and accordingly utilities, of VGAM265 correlate with, and may be deduced from, the identity of the target genes which VGAM265 binds and inhibits, and the function of these target genes, as elaborated hereinbelow.

Apolipoprotein C-IV (APOC4, Accession NM_{—}001646) is a VGAM265 host target gene. APOC4 BINDING SITE is HOST TARGET binding site found in the 3′ untranslated region of mRNA encoded by APOC4, corresponding to a HOST TARGET binding site such as BINDING SITE I, BINDING SITE II or BINDING SITE III. Table 2 illustrates the complementarity of the nucleotide sequences of APOC4 BINDING SITE, designated SEQ ID:841, to the nucleotide sequence of VGAM265 RNA, herein designated VGAM RNA, also designated SEQ ID:600.

A function of VGAM265 is therefore inhibition of Apolipoprotein C-IV (APOC4, Accession NM_{—}001646). Accordingly, utilities of VGAM265 include diagnosis, prevention and treatment of diseases and clinical conditions associated with APOC4. DKFZP434P0721 (Accession XM_{—}033181) is another VGAM265 host target gene. DKFZP434P0721 BINDING SITE is HOST TARGET binding site found in the 3′ untranslated region of mRNA encoded by DKFZP434P0721, corresponding to a HOST TARGET binding site such as BINDING SITE I, BINDING SITE II or BINDING SITE III. Table 2 illustrates the complementarity of the nucleotide sequences of DKFZP434P0721 BINDING SITE, designated SEQ ID:2664, to the nucleotide sequence of VGAM265 RNA, herein designated VGAM RNA, also designated SEQ ID:600.

Another function of VGAM265 is therefore inhibition of DKFZP434P0721 (Accession XM_{—}033181). Accordingly, utilities of VGAM265 include diagnosis, prevention and treatment of diseases and clinical conditions associated with DKFZP434P0721. FLJ14082 (Accession NM_{—}025024) is another VGAM265 host target gene. FLJ14082 BINDING SITE is HOST TARGET binding site found in the 3′ untranslated region of mRNA encoded by FLJ14082, corresponding to a HOST TARGET binding site such as BINDING SITE I, BINDING SITE II or BINDING SITE III. Table 2 illustrates the complementarity of the nucleotide sequences of FLJ14082 BINDING SITE, designated SEQ ID:2129, to the nucleotide sequence of VGAM265 RNA, herein designated VGAM RNA, also designated SEQ ID:600.

Another function of VGAM265 is therefore inhibition of FLJ14082 (Accession NM_{—}025024). Accordingly, utilities of VGAM265 include diagnosis, prevention and treatment of diseases and clinical conditions associated with FLJ14082. FIG. 1 further provides a conceptual description of a novel bioinformatically detected viral gene of the present invention, referred to here as Viral Genomic Address Messenger 266 (VGAM266) viral gene, which modulates expression of respective host target genes thereof, the function and utility of which host target genes is known in the art.

VGAM266 is a novel bioinformatically detected regulatory, non protein coding, viral micro RNA (miRNA) gene. The method by which VGAM266 was detected is described hereinabove with reference to FIGS. 1-8.

VGAM266 gene, herein designated VGAM GENE, is a viral gene contained in the genome of Vaccinia Virus. VGAM266 host target gene, herein designated VGAM HOST TARGET GENE, is a human gene contained in the human genome.

VGAM266 gene encodes a VGAM266 precursor RNA, herein designated VGAM PRECURSOR RNA. Similar to other miRNA genes, and unlike most ordinary genes, VGAM266 precursor RNA does not encode a protein. A nucleotide sequence identical or highly similar to the nucleotide sequence of VGAM266 precursor RNA is designated SEQ ID:252, and is provided hereinbelow with reference to the sequence listing part. Nucleotide sequence SEQ ID:252 is located at position 149215 relative to the genome of Vaccinia Virus.

VGAM266 precursor RNA folds onto itself, forming VGAM266 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM266 folded precursor RNA into VGAM266 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM266 RNA is designated SEQ ID:601, and is provided hereinbelow with reference to the sequence listing part.

VGAM266 host target gene, herein designated VGAM HOST TARGET GENE, encodes a corresponding messenger RNA, VGAM266 host target RNA, herein designated VGAM HOST TARGET RNA. VGAM266 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM266 RNA, herein designated VGAM RNA, binds complementarily to one or more host target binding sites located in untranslated regions of VGAM266 host target RNA, herein designated VGAM HOST TARGET RNA. This complementary binding is due to the fact that the nucleotide sequence of VGAM266 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM266 RNA, herein designated VGAM RNA, may have a different number of host target binding sites in untranslated regions of a VGAM266 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM266 RNA, herein designated VGAM RNA, to host target binding sites on VGAM266 host target RNA, herein designated VGAM HOST TARGET RNA, such as BINDING SITE I, BINDING SITE II and BINDING SITE III, inhibits translation of VGAM266 host target RNA into VGAM266 host target protein, herein designated VGAM HOST TARGET PROTEIN. VGAM host target protein is therefore outlined by a broken line.

It is appreciated that VGAM266 host target gene, herein designated VGAM HOST TARGET GENE, in fact represents a plurality of VGAM266 host target genes. The mRNA of each one of this plurality of VGAM266 host target genes comprises one or more host target binding sites, each having a nucleotide sequence which is at least partly complementary to VGAM266 RNA, herein designated VGAM RNA, and which when bound by VGAM266 RNA causes inhibition of translation of respective one or more VGAM266 host target proteins.

It is further appreciated by one skilled in the art that the mode of translational inhibition illustrated by FIG. 1 with specific reference to translational inhibition exerted by VGAM266 gene, herein designated VGAM GENE, on one or more VGAM266 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM266 is inhibition of expression of host target genes, as part of a novel viral mechanism of attacking a host. Accordingly, utilities of VGAM266 include diagnosis, prevention and treatment of viral infection by Vaccinia Virus. Specific functions, and accordingly utilities, of VGAM266 correlate with, and may be deduced from, the identity of the host target genes which VGAM266 binds and inhibits, and the function of these host target genes, as elaborated hereinbelow.

Nucleotide sequences of the VGAM266 precursor RNA, herein designated VGAM PRECURSOR RNA, and of the ‘diced’ VGAM266 RNA, herein designated VGAM RNA, and a schematic representation of the secondary folding of VGAM266 folded precursor RNA, herein designated VGAM FOLDED PRECURSOR RNA, of VGAM266 are further described hereinbelow with reference to Table 1.

Nucleotide sequences of host target binding sites, such as BINDING SITE-I, BINDING SITE-II and BINDING SITE-III of FIG. 1, found on VGAM266 host target RNA, and schematic representation of the complementarity of each of these host target binding sites to VGAM266 RNA, herein designated VGAM RNA, are described hereinbelow with reference to Table 2.

As mentioned hereinabove with reference to FIG. 1, a function of VGAM266 gene, herein designated VGAM is inhibition of expression of VGAM266 target genes. It is appreciated that specific functions, and accordingly utilities, of VGAM266 correlate with, and may be deduced from, the identity of the target genes which VGAM266 binds and inhibits, and the function of these target genes, as elaborated hereinbelow.

LIM Domain Only 7 (LMO7, Accession NM_{—}005358) is a VGAM266 host target gene. LMO7 BINDING SITE1 and LMO7 BINDING SITE2 are HOST TARGET binding sites found in untranslated regions of mRNA encoded by LMO7, corresponding to HOST TARGET binding sites such as BINDING SITE I, BINDING SITE II or BINDING SITE III. Table 2 illustrates the complementarity of the nucleotide sequences of LMO7 BINDING SITE1 and LMO7 BINDING SITE2, designated SEQ ID:1202 and SEQ ID:1643 respectively, to the nucleotide sequence of VGAM266 RNA, herein designated VGAM RNA, also designated SEQ ID:601.

A function of VGAM266 is therefore inhibition of LIM Domain Only 7 (LMO7, Accession NM_{—}005358). Accordingly, utilities of VGAM266 include diagnosis, prevention and treatment of diseases and clinical conditions associated with LMO7. KIAA1671 (Accession XM_{—}037809) is another VGAM266 host target gene. KIAA1671 BINDING SITE is HOST TARGET binding site found in the 3′ untranslated region of mRNA encoded by KIAA1671, corresponding to a HOST TARGET binding site such as BINDING SITE I, BINDING SITE II or BINDING SITE III. Table 2 illustrates the complementarity of the nucleotide sequences of KIAA1671 BINDING SITE, designated SEQ ID:2726, to the nucleotide sequence of VGAM266 RNA, herein designated VGAM RNA, also designated SEQ ID:601.

Another function of VGAM266 is therefore inhibition of KIAA1671 (Accession XM_{—}037809). Accordingly, utilities of VGAM266 include diagnosis, prevention and treatment of diseases and clinical conditions associated with KIAA1671. Sema Domain, Seven Thrombospondin Repeats (type 1 and type 1-like), Transmembrane Domain (TM) and Short Cytoplasmic Domain, (semaphorin) 5A (SEMA5A, Accession NM_{—}003966) is another VGAM266 host target gene. SEMA5A BINDING SITE is HOST TARGET binding site found in the 3′ untranslated region of mRNA encoded by SEMA5A, corresponding to a HOST TARGET binding site such as BINDING SITE I, BINDING SITE II or BINDING SITE III. Table 2 illustrates the complementarity of the nucleotide sequences of SEMA5A BINDING SITE, designated SEQ ID: 1075, to the nucleotide sequence of VGAM266 RNA, herein designated VGAM RNA, also designated SEQ ID:601.

Another function of VGAM266 is therefore inhibition of Sema Domain, Seven Thrombospondin Repeats (type 1 and type 1-like), Transmembrane Domain (TM) and Short Cytoplasmic Domain, (semaphorin) 5A (SEMA5A, Accession NM_{—}003966). Accordingly, utilities of VGAM266 include diagnosis, prevention and treatment of diseases and clinical conditions associated with SEMA5A. LOC127133 (Accession XM_{—}059114) is another VGAM266 host target gene. LOC127133 BINDING SITE is HOST TARGET binding site found in the 3′ untranslated region of mRNA encoded by LOC127133, corresponding to a HOST TARGET binding site such as BINDING SITE I, BINDING SITE II or BINDING SITE III. Table 2 illustrates the complementarity of the nucleotide sequences of LOC127133 BINDING SITE, designated SEQ ID:3004, to the nucleotide sequence of VGAM266 RNA, herein designated VGAM RNA, also designated SEQ ID:601.

Another function of VGAM266 is therefore inhibition of LOC127133 (Accession XM_{—}059114). Accordingly, utilities of VGAM266 include diagnosis, prevention and treatment of diseases and clinical conditions associated with LOC127133. LOC143154 (Accession XM_{—}084441) is another VGAM266 host target gene. LOC143154 BINDING SITE is HOST TARGET binding site found in the 3′ untranslated region of mRNA encoded by LOC143154, corresponding to a HOST TARGET binding site such as BINDING SITE I, BINDING SITE II or BINDING SITE III. Table 2 illustrates the complementarity of the nucleotide sequences of LOC143154 BINDING SITE, designated SEQ ID:3052, to the nucleotide sequence of VGAM266 RNA, herein designated VGAM RNA, also designated SEQ ID:601.

Another function of VGAM266 is therefore inhibition of LOC143154 (Accession XM_{—}084441). Accordingly, utilities of VGAM266 include diagnosis, prevention and treatment of diseases and clinical conditions associated with LOC143154. LOC219294 (Accession XM_{—}167566) is another VGAM266 host target gene. LOC219294 BINDING SITE is HOST TARGET binding site found in the 3′ untranslated region of mRNA encoded by LOC219294, corresponding to a HOST TARGET binding site such as BINDING SITE I, BINDING SITE II or BINDING SITE III. Table 2 illustrates the complementarity of the nucleotide sequences of LOC219294 BINDING SITE, designated SEQ ID:3587, to the nucleotide sequence of VGAM266 RNA, herein designated VGAM RNA, also designated SEQ ID:601.

Another function of VGAM266 is therefore inhibition of LOC219294 (Accession XM_{—}167566). Accordingly, utilities of VGAM266 include diagnosis, prevention and treatment of diseases and clinical conditions associated with LOC219294. FIG. 1 further provides a conceptual description of a novel bioinformatically detected viral gene of the present invention, referred to here as Viral Genomic Address Messenger 267 (VGAM267) viral gene, which modulates expression of respective host target genes thereof, the function and utility of which host target genes is known in the art.

VGAM267 is a novel bioinformatically detected regulatory, non protein coding, viral micro RNA (miRNA) gene. The method by which VGAM267 was detected is described hereinabove with reference to FIGS. 1-8.

VGAM267 gene, herein designated VGAM GENE, is a viral gene contained in the genome of Vaccinia Virus. VGAM267 host target gene, herein designated VGAM HOST TARGET GENE, is a human gene contained in the human genome.

VGAM267 gene encodes a VGAM267 precursor RNA, herein designated VGAM PRECURSOR RNA. Similar to other miRNA genes, and unlike most ordinary genes, VGAM267 precursor RNA does not encode a protein. A nucleotide sequence identical or highly similar to the nucleotide sequence of VGAM267 precursor RNA is designated SEQ ID:253, and is provided hereinbelow with reference to the sequence listing part. Nucleotide sequence SEQ ID:253 is located at position 148793 relative to the genome of Vaccinia Virus.

VGAM267 precursor RNA folds onto itself, forming VGAM267 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM267 folded precursor RNA into VGAM267 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM267 RNA is designated SEQ ID:602, and is provided hereinbelow with reference to the sequence listing part.

VGAM267 host target gene, herein designated VGAM HOST TARGET GENE, encodes a corresponding messenger RNA, VGAM267 host target RNA, herein designated VGAM HOST TARGET RNA. VGAM267 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM267 RNA, herein designated VGAM RNA, binds complementarily to one or more host target binding sites located in untranslated regions of VGAM267 host target RNA, herein designated VGAM HOST TARGET RNA. This complementary binding is due to the fact that the nucleotide sequence of VGAM267 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM267 RNA, herein designated VGAM RNA, may have a different number of host target binding sites in untranslated regions of a VGAM267 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM267 RNA, herein designated VGAM RNA, to host target binding sites on VGAM267 host target RNA, herein designated VGAM HOST TARGET RNA, such as BINDING SITE I, BINDING SITE II and BINDING SITE III, inhibits translation of VGAM267 host target RNA into VGAM267 host target protein, herein designated VGAM HOST TARGET PROTEIN. VGAM host target protein is therefore outlined by a broken line.

It is appreciated that VGAM267 host target gene, herein designated VGAM HOST TARGET GENE, in fact represents a plurality of VGAM267 host target genes. The mRNA of each one of this plurality of VGAM267 host target genes comprises one or more host target binding sites, each having a nucleotide sequence which is at least partly complementary to VGAM267 RNA, herein designated VGAM RNA, and which when bound by VGAM267 RNA causes inhibition of translation of respective one or more VGAM267 host target proteins.

It is further appreciated by one skilled in the art that the mode of translational inhibition illustrated by FIG. 1 with specific reference to translational inhibition exerted by VGAM267 gene, herein designated VGAM GENE, on one or more VGAM267 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM267 is inhibition of expression of host target genes, as part of a novel viral mechanism of attacking a host. Accordingly, utilities of VGAM267 include diagnosis, prevention and treatment of viral infection by Vaccinia Virus. Specific functions, and accordingly utilities, of VGAM267 correlate with, and may be deduced from, the identity of the host target genes which VGAM267 binds and inhibits, and the function of these host target genes, as elaborated hereinbelow.

Nucleotide sequences of the VGAM267 precursor RNA, herein designated VGAM PRECURSOR RNA, and of the ‘diced’ VGAM267 RNA, herein designated VGAM RNA, and a schematic representation of the secondary folding of VGAM267 folded precursor RNA, herein designated VGAM FOLDED PRECURSOR RNA, of VGAM267 are further described hereinbelow with reference to Table 1.

Nucleotide sequences of host target binding sites, such as BINDING SITE-I, BINDING SITE-II and BINDING SITE-III of FIG. 1, found on VGAM267 host target RNA, and schematic representation of the complementarity of each of these host target binding sites to VGAM267 RNA, herein designated VGAM RNA, are described hereinbelow with reference to Table 2.

As mentioned hereinabove with reference to FIG. 1, a function of VGAM267 gene, herein designated VGAM is inhibition of expression of VGAM267 target genes. It is appreciated that specific functions, and accordingly utilities, of VGAM267 correlate with, and may be deduced from, the identity of the target genes which VGAM267 binds and inhibits, and the function of these target genes, as elaborated hereinbelow.

Glycoprotein A Repetitions Predominant (GARP, Accession NM_{—}005512) is a VGAM267 host target gene. GARP BINDING SITE is HOST TARGET binding site found in the 3′ untranslated region of mRNA encoded by GARP, corresponding to a HOST TARGET binding site such as BINDING SITE I, BINDING SITE II or BINDING SITE III. Table 2 illustrates the complementarity of the nucleotide sequences of GARP BINDING SITE, designated SEQ ID:1221, to the nucleotide sequence of VGAM267 RNA, herein designated VGAM RNA, also designated SEQ ID:602.

A function of VGAM267 is therefore inhibition of Glycoprotein A Repetitions Predominant (GARP, Accession NM_{—}005512). Accordingly, utilities of VGAM267 include diagnosis, prevention and treatment of diseases and clinical conditions associated with GARP. FLJ30213 (Accession NM_{—}145008) is another VGAM267 host target gene. FLJ30213 BINDING SITE is HOST TARGET binding site found in the 3′ untranslated region of mRNA encoded by FLJ30213, corresponding to a HOST TARGET binding site such as BINDING SITE I, BINDING SITE II or BINDING SITE III. Table 2 illustrates the complementarity of the nucleotide sequences of FLJ30213 BINDING SITE, designated SEQ ID:2507, to the nucleotide sequence of VGAM267 RNA, herein designated VGAM RNA, also designated SEQ ID:602.

Another function of VGAM267 is therefore inhibition of FLJ30213 (Accession NM_{—}145008). Accordingly, utilities of VGAM267 include diagnosis, prevention and treatment of diseases and clinical conditions associated with FLJ30213. LOC196510 (Accession XM_{—}113738) is another VGAM267 host target gene. LOC196510 BINDING SITE is HOST TARGET binding site found in the 3′ untranslated region of mRNA encoded by LOC196510, corresponding to a HOST TARGET binding site such as BINDING SITE I, BINDING SITE II or BINDING SITE III. Table 2 illustrates the complementarity of the nucleotide sequences of LOC196510 BINDING SITE, designated SEQ ID:3415, to the nucleotide sequence of VGAM267 RNA, herein designated VGAM RNA, also designated SEQ ID:602.

Another function of VGAM267 is therefore inhibition of LOC196510 (Accession XM_{—}113738). Accordingly, utilities of VGAM267 include diagnosis, prevention and treatment of diseases and clinical conditions associated with LOC196510. LOC200220 (Accession XM_{—}114157) is another VGAM267 host target gene. LOC200220 BINDING SITE is HOST TARGET binding site found in the 3′ untranslated region of mRNA encoded by LOC200220, corresponding to a HOST TARGET binding site such as BINDING SITE I, BINDING SITE II or BINDING SITE III. Table 2 illustrates the complementarity of the nucleotide sequences of LOC200220 BINDING SITE, designated SEQ ID:3444, to the nucleotide sequence of VGAM267 RNA, herein designated VGAM RNA, also designated SEQ ID:602.

Another function of VGAM267 is therefore inhibition of LOC200220 (Accession XM_{—}114157). Accordingly, utilities of VGAM267 include diagnosis, prevention and treatment of diseases and clinical conditions associated with LOC200220. LOC220930 (Accession XM_{—}167624) is another VGAM267 host target gene. LOC220930 BINDING SITE is HOST TARGET binding site found in the 3′ untranslated region of mRNA encoded by LOC220930, corresponding to a HOST TARGET binding site such as BINDING SITE I, BINDING SITE II or BINDING SITE III. Table 2 illustrates the complementarity of the nucleotide sequences of LOC220930 BINDING SITE, designated SEQ ID:3591, to the nucleotide sequence of VGAM267 RNA, herein designated VGAM RNA, also designated SEQ ID:602.

Another function of VGAM267 is therefore inhibition of LOC220930 (Accession XM_{—}167624). Accordingly, utilities of VGAM267 include diagnosis, prevention and treatment of diseases and clinical conditions associated with LOC220930. FIG. 1 further provides a conceptual description of a novel bioinformatically detected viral gene of the present invention, referred to here as Viral Genomic Address Messenger 268 (VGAM268) viral gene, which modulates expression of respective host target genes thereof, the function and utility of which host target genes is known in the art.

VGAM268 is a novel bioinformatically detected regulatory, non protein coding, viral micro RNA (miRNA) gene. The method by which VGAM268 was detected is described hereinabove with reference to FIGS. 1-8.

VGAM268 gene, herein designated VGAM GENE, is a viral gene contained in the genome of Vaccinia Virus. VGAM268 host target gene, herein designated VGAM HOST TARGET GENE, is a human gene contained in the human genome.

VGAM268 gene encodes a VGAM268 precursor RNA, herein designated VGAM PRECURSOR RNA. Similar to other miRNA genes, and unlike most ordinary genes, VGAM268 precursor RNA does not encode a protein. A nucleotide sequence identical or highly similar to the nucleotide sequence of VGAM268 precursor RNA is designated SEQ ID:254, and is provided hereinbelow with reference to the sequence listing part. Nucleotide sequence SEQ ID:254 is located at position 153199 relative to the genome of Vaccinia Virus.

VGAM268 precursor RNA folds onto itself, forming VGAM268 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM268 folded precursor RNA into VGAM268 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM268 RNA is designated SEQ ID:603, and is provided hereinbelow with reference to the sequence listing part.

VGAM268 host target gene, herein designated VGAM HOST TARGET GENE, encodes a corresponding messenger RNA, VGAM268 host target RNA, herein designated VGAM HOST TARGET RNA. VGAM268 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM268 RNA, herein designated VGAM RNA, binds complementarily to one or more host target binding sites located in untranslated regions of VGAM268 host target RNA, herein designated VGAM HOST TARGET RNA. This complementary binding is due to the fact that the nucleotide sequence of VGAM268 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM268 RNA, herein designated VGAM RNA, may have a different number of host target binding sites in untranslated regions of a VGAM268 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM268 RNA, herein designated VGAM RNA, to host target binding sites on VGAM268 host target RNA, herein designated VGAM HOST TARGET RNA, such as BINDING SITE I, BINDING SITE II and BINDING SITE III, inhibits translation of VGAM268 host target RNA into VGAM268 host target protein, herein designated VGAM HOST TARGET PROTEIN. VGAM host target protein is therefore outlined by a broken line.

It is appreciated that VGAM268 host target gene, herein designated VGAM HOST TARGET GENE, in fact represents a plurality of VGAM268 host target genes. The mRNA of each one of this plurality of VGAM268 host target genes comprises one or more host target binding sites, each having a nucleotide sequence which is at least partly complementary to VGAM268 RNA, herein designated VGAM RNA, and which when bound by VGAM268 RNA causes inhibition of translation of respective one or more VGAM268 host target proteins.

It is further appreciated by one skilled in the art that the mode of translational inhibition illustrated by FIG. 1 with specific reference to translational inhibition exerted by VGAM268 gene, herein designated VGAM GENE, on one or more VGAM268 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM268 is inhibition of expression of host target genes, as part of a novel viral mechanism of attacking a host. Accordingly, utilities of VGAM268 include diagnosis, prevention and treatment of viral infection by Vaccinia Virus. Specific functions, and accordingly utilities, of VGAM268 correlate with, and may be deduced from, the identity of the host target genes which VGAM268 binds and inhibits, and the function of these host target genes, as elaborated hereinbelow.

Nucleotide sequences of the VGAM268 precursor RNA, herein designated VGAM PRECURSOR RNA, and of the ‘diced’ VGAM268 RNA, herein designated VGAM RNA, and a schematic representation of the secondary folding of VGAM268 folded precursor RNA, herein designated VGAM FOLDED PRECURSOR RNA, of VGAM268 are further described hereinbelow with reference to Table 1.

Nucleotide sequences of host target binding sites, such as BINDING SITE-I, BINDING SITE-II and BINDING SITE-III of FIG. 1, found on VGAM268 host target RNA, and schematic representation of the complementarity of each of these host target binding sites to VGAM268 RNA, herein designated VGAM RNA, are described hereinbelow with reference to Table 2.

As mentioned hereinabove with reference to FIG. 1, a function of VGAM268 gene, herein designated VGAM is inhibition of expression of VGAM268 target genes. It is appreciated that specific functions, and accordingly utilities, of VGAM268 correlate with, and may be deduced from, the identity of the target genes which VGAM268 binds and inhibits, and the function of these target genes, as elaborated hereinbelow.

Zinc Finger Protein 161 (ZNF161, Accession NM_{—}007146) is a VGAM268 host target gene. ZNF161 BINDING SITE is HOST TARGET binding site found in the 3′ untranslated region of mRNA encoded by ZNF161, corresponding to a HOST TARGET binding site such as BINDING SITE I, BINDING SITE II or BINDING SITE III. Table 2 illustrates the complementarity of the nucleotide sequences of ZNF161 BINDING SITE, designated SEQ ID:1359, to the nucleotide sequence of VGAM268 RNA, herein designated VGAM RNA, also designated SEQ ID:603.

A function of VGAM268 is therefore inhibition of Zinc Finger Protein 161 (ZNF161, Accession NM_{—}007146). Accordingly, utilities of VGAM268 include diagnosis, prevention and treatment of diseases and clinical conditions associated with ZNF161. DKFZP566D193 (Accession XM_{—}098173) is another VGAM268 host target gene. DKFZP566D193 BINDING SITE is HOST TARGET binding site found in the 3′ untranslated region of mRNA encoded by DKFZP566D193, corresponding to a HOST TARGET binding site such as BINDING SITE I, BINDING SITE II or BINDING SITE III. Table 2 illustrates the complementarity of the nucleotide sequences of DKFZP566D193 BINDING SITE, designated SEQ ID:3353, to the nucleotide sequence of VGAM268 RNA, herein designated VGAM RNA, also designated SEQ ID:603.

Another function of VGAM268 is therefore inhibition of DKFZP566D193 (Accession XM_{—}098173). Accordingly, utilities of VGAM268 include diagnosis, prevention and treatment of diseases and clinical conditions associated with DKFZP566D193. LOC200574 (Accession XM_{—}114264) is another VGAM268 host target gene. LOC200574 BINDING SITE is HOST TARGET binding site found in the 3′ untranslated region of mRNA encoded by LOC200574, corresponding to a HOST TARGET binding site such as BINDING SITE I, BINDING SITE II or BINDING SITE III. Table 2 illustrates the complementarity of the nucleotide sequences of LOC200574 BINDING SITE, designated SEQ ID:3450, to the nucleotide sequence of VGAM268 RNA, herein designated VGAM RNA, also designated SEQ ID:603.

Another function of VGAM268 is therefore inhibition of LOC200574 (Accession XM_{—}114264). Accordingly, utilities of VGAM268 include diagnosis, prevention and treatment of diseases and clinical conditions associated with LOC200574. FIG. 1 further provides a conceptual description of a novel bioinformatically detected viral gene of the present invention, referred to here as Viral Genomic Address Messenger 269 (VGAM269) viral gene, which modulates expression of respective host target genes thereof, the function and utility of which host target genes is known in the art.

VGAM269 is a novel bioinformatically detected regulatory, non protein coding, viral micro RNA (miRNA) gene. The method by which VGAM269 was detected is described hereinabove with reference to FIGS. 1-8.

VGAM269 gene, herein designated VGAM GENE, is a viral gene contained in the genome of Vaccinia Virus. VGAM269 host target gene, herein designated VGAM HOST TARGET GENE, is a human gene contained in the human genome.

VGAM269 gene encodes a VGAM269 precursor RNA, herein designated VGAM PRECURSOR RNA. Similar to other miRNA genes, and unlike most ordinary genes, VGAM269 precursor RNA does not encode a protein. A nucleotide sequence identical or highly similar to the nucleotide sequence of VGAM269 precursor RNA is designated SEQ ID:255, and is provided hereinbelow with reference to the sequence listing part. Nucleotide sequence SEQ ID:255 is located at position 153361 relative to the genome of Vaccinia Virus.

VGAM269 precursor RNA folds onto itself, forming VGAM269 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM269 folded precursor RNA into VGAM269 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM269 RNA is designated SEQ ID:604, and is provided hereinbelow with reference to the sequence listing part.

VGAM269 host target gene, herein designated VGAM HOST TARGET GENE, encodes a corresponding messenger RNA, VGAM269 host target RNA, herein designated VGAM HOST TARGET RNA. VGAM269 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM269 RNA, herein designated VGAM RNA, binds complementarily to one or more host target binding sites located in untranslated regions of VGAM269 host target RNA, herein designated VGAM HOST TARGET RNA. This complementary binding is due to the fact that the nucleotide sequence of VGAM269 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM269 RNA, herein designated VGAM RNA, may have a different number of host target binding sites in untranslated regions of a VGAM269 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM269 RNA, herein designated VGAM RNA, to host target binding sites on VGAM269 host target RNA, herein designated VGAM HOST TARGET RNA, such as BINDING SITE I, BINDING SITE II and BINDING SITE III, inhibits translation of VGAM269 host target RNA into VGAM269 host target protein, herein designated VGAM HOST TARGET PROTEIN. VGAM host target protein is therefore outlined by a broken line.

It is appreciated that VGAM269 host target gene, herein designated VGAM HOST TARGET GENE, in fact represents a plurality of VGAM269 host target genes. The mRNA of each one of this plurality of VGAM269 host target genes comprises one or more host target binding sites, each having a nucleotide sequence which is at least partly complementary to VGAM269 RNA, herein designated VGAM RNA, and which when bound by VGAM269 RNA causes inhibition of translation of respective one or more VGAM269 host target proteins.

It is further appreciated by one skilled in the art that the mode of translational inhibition illustrated by FIG. 1 with specific reference to translational inhibition exerted by VGAM269 gene, herein designated VGAM GENE, on one or more VGAM269 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM269 is inhibition of expression of host target genes, as part of a novel viral mechanism of attacking a host. Accordingly, utilities of VGAM269 include diagnosis, prevention and treatment of viral infection by Vaccinia Virus. Specific functions, and accordingly utilities, of VGAM269 correlate with, and may be deduced from, the identity of the host target genes which VGAM269 binds and inhibits, and the function of these host target genes, as elaborated hereinbelow.

Nucleotide sequences of the VGAM269 precursor RNA, herein designated VGAM PRECURSOR RNA, and of the ‘diced’ VGAM269 RNA, herein designated VGAM RNA, and a schematic representation of the secondary folding of VGAM269 folded precursor RNA, herein designated VGAM FOLDED PRECURSOR RNA, of VGAM269 are further described hereinbelow with reference to Table 1.

Nucleotide sequences of host target binding sites, such as BINDING SITE-I, BINDING SITE-II and BINDING SITE-III of FIG. 1, found on VGAM269 host target RNA, and schematic representation of the complementarity of each of these host target binding sites to VGAM269 RNA, herein designated VGAM RNA, are described hereinbelow with reference to Table 2.

As mentioned hereinabove with reference to FIG. 1, a function of VGAM269 gene, herein designated VGAM is inhibition of expression of VGAM269 target genes. It is appreciated that specific functions, and accordingly utilities, of VGAM269 correlate with, and may be deduced from, the identity of the target genes which VGAM269 binds and inhibits, and the function of these target genes, as elaborated hereinbelow.

RAD50 Homolog (S. cerevisiae) (RAD50, Accession NM_{—}005732) is a VGAM269 host target gene. RAD50 BINDING SITE1 and RAD50 BINDING SITE2 are HOST TARGET binding sites found in untranslated regions of mRNA encoded by RAD50, corresponding to HOST TARGET binding sites such as BINDING SITE I, BINDING SITE II or BINDING SITE III. Table 2 illustrates the complementarity of the nucleotide sequences of RAD50 BINDING SITE1 and RAD50 BINDING SITE2, designated SEQ ID:1240 and SEQ ID:2420 respectively, to the nucleotide sequence of VGAM269 RNA, herein designated VGAM RNA, also designated SEQ ID:604.

A function of VGAM269 is therefore inhibition of RAD50 Homolog (S. cerevisiae) (RAD50, Accession NM_{—}005732), a gene which is involved in dna double-strand break repair (dsbr). Accordingly, utilities of VGAM269 include diagnosis, prevention and treatment of diseases and clinical conditions associated with RAD50. The function of RAD50 and its association with various diseases and clinical conditions, has been established by previous studies, as described hereinabove with reference to VGAM119. Tripartite Motif-containing 9 (TRIM9, Accession NM_{—}015163) is another VGAM269 host target gene. TRIM9 BINDING SITE is HOST TARGET binding site found in the 3′ untranslated region of mRNA encoded by TRIM9, corresponding to a HOST TARGET binding site such as BINDING SITE I, BINDING SITE II or BINDING SITE III. Table 2 illustrates the complementarity of the nucleotide sequences of TRIM9 BINDING SITE, designated SEQ ID:1611, to the nucleotide sequence of VGAM269 RNA, herein designated VGAM RNA, also designated SEQ ID:604.

Another function of VGAM269 is therefore inhibition of Tripartite Motif-containing 9 (TRIM9, Accession NM_{—}015163), a gene which may function as a positive regulator for mannosylphosphate transferase and is required to mediate mannosylphosphate transfer in both the core and outer chain portions of n-linked. oligosaccharides. Accordingly, utilities of VGAM269 include diagnosis, prevention and treatment of diseases and clinical conditions associated with TRIM9. The function of TRIM9 and its association with various diseases and clinical conditions, has been established by previous studies, as described hereinabove with reference to VGAM101. FLJ13902 (Accession NM_{—}024653) is another VGAM269 host target gene. FLJ13902 BINDING SITE is HOST TARGET binding site found in the 3′ untranslated region of mRNA encoded by FLJ13902, corresponding to a HOST TARGET binding site such as BINDING SITE I, BINDING SITE II or BINDING SITE III. Table 2 illustrates the complementarity of the nucleotide sequences of FLJ13902 BINDING SITE, designated SEQ ID:2076, to the nucleotide sequence of VGAM269 RNA, herein designated VGAM RNA, also designated SEQ ID:604.

Another function of VGAM269 is therefore inhibition of FLJ13902 (Accession NM_{—}024653). Accordingly, utilities of VGAM269 include diagnosis, prevention and treatment of diseases and clinical conditions associated with FLJ13902. KIAA0650 (Accession XM_{—}113962) is another VGAM269 host target gene. KIAA0650 BINDING SITE is HOST TARGET binding site found in the 3′ untranslated region of mRNA encoded by KIAA0650, corresponding to a HOST TARGET binding site such as BINDING SITE I, BINDING SITE II or BINDING SITE III. Table 2 illustrates the complementarity of the nucleotide sequences of KIAA0650 BINDING SITE, designated SEQ ID:3427, to the nucleotide sequence of VGAM269 RNA, herein designated VGAM RNA, also designated SEQ ID:604.

Another function of VGAM269 is therefore inhibition of KIAA0650 (Accession XM_{—}113962). Accordingly, utilities of VGAM269 include diagnosis, prevention and treatment of diseases and clinical conditions associated with KIAA0650. MGC16075 (Accession NM_{—}032761) is another VGAM269 host target gene. MGC16075 BINDING SITE is HOST TARGET binding site found in the 3′ untranslated region of mRNA encoded by MGC16075, corresponding to a HOST TARGET binding site such as BINDING SITE I, BINDING SITE II or BINDING SITE III. Table 2 illustrates the complementarity of the nucleotide sequences of MGC16075 BINDING SITE, designated SEQ ID:2277, to the nucleotide sequence of VGAM269 RNA, herein designated VGAM RNA, also designated SEQ ID:604.

Another function of VGAM269 is therefore inhibition of MGC16075 (Accession NM_{—}032761). Accordingly, utilities of VGAM269 include diagnosis, prevention and treatment of diseases and clinical conditions associated with MGC16075. FIG. 1 further provides a conceptual description of a novel bioinformatically detected viral gene of the present invention, referred to here as Viral Genomic Address Messenger 270 (VGAM270) viral gene, which modulates expression of respective host target genes thereof, the function and utility of which host target genes is known in the art.

VGAM270 is a novel bioinformatically detected regulatory, non protein coding, viral micro RNA (miRNA) gene. The method by which VGAM270 was detected is described hereinabove with reference to FIGS. 1-8.

VGAM270 gene, herein designated VGAM GENE, is a viral gene contained in the genome of Vaccinia Virus. VGAM270 host target gene, herein designated VGAM HOST TARGET GENE, is a human gene contained in the human genome.

VGAM270 gene encodes a VGAM270 precursor RNA, herein designated VGAM PRECURSOR RNA. Similar to other miRNA genes, and unlike most ordinary genes, VGAM270 precursor RNA does not encode a protein. A nucleotide sequence identical or highly similar to the nucleotide sequence of VGAM270 precursor RNA is designated SEQ ID:256, and is provided hereinbelow with reference to the sequence listing part. Nucleotide sequence SEQ ID:256 is located at position 153717 relative to the genome of Vaccinia Virus.

VGAM270 precursor RNA folds onto itself, forming VGAM270 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM270 folded precursor RNA into VGAM270 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM270 RNA is designated SEQ ID:605, and is provided hereinbelow with reference to the sequence listing part.

VGAM270 host target gene, herein designated VGAM HOST TARGET GENE, encodes a corresponding messenger RNA, VGAM270 host target RNA, herein designated VGAM HOST TARGET RNA. VGAM270 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM270 RNA, herein designated VGAM RNA, binds complementarily to one or more host target binding sites located in untranslated regions of VGAM270 host target RNA, herein designated VGAM HOST TARGET RNA. This complementary binding is due to the fact that the nucleotide sequence of VGAM270 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM270 RNA, herein designated VGAM RNA, may have a different number of host target binding sites in untranslated regions of a VGAM270 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM270 RNA, herein designated VGAM RNA, to host target binding sites on VGAM270 host target RNA, herein designated VGAM HOST TARGET RNA, such as BINDING SITE I, BINDING SITE II and BINDING SITE III, inhibits translation of VGAM270 host target RNA into VGAM270 host target protein, herein designated VGAM HOST TARGET PROTEIN. VGAM host target protein is therefore outlined by a broken line.

It is appreciated that VGAM270 host target gene, herein designated VGAM HOST TARGET GENE, in fact represents a plurality of VGAM270 host target genes. The mRNA of each one of this plurality of VGAM270 host target genes comprises one or more host target binding sites, each having a nucleotide sequence which is at least partly complementary to VGAM270 RNA, herein designated VGAM RNA, and which when bound by VGAM270 RNA causes inhibition of translation of respective one or more VGAM270 host target proteins.

It is further appreciated by one skilled in the art that the mode of translational inhibition illustrated by FIG. 1 with specific reference to translational inhibition exerted by VGAM270 gene, herein designated VGAM GENE, on one or more VGAM270 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM270 is inhibition of expression of host target genes, as part of a novel viral mechanism of attacking a host. Accordingly, utilities of VGAM270 include diagnosis, prevention and treatment of viral infection by Vaccinia Virus. Specific functions, and accordingly utilities, of VGAM270 correlate with, and may be deduced from, the identity of the host target genes which VGAM270 binds and inhibits, and the function of these host target genes, as elaborated hereinbelow.

Nucleotide sequences of the VGAM270 precursor RNA, herein designated VGAM PRECURSOR RNA, and of the ‘diced’ VGAM270 RNA, herein designated VGAM RNA, and a schematic representation of the secondary folding of VGAM270 folded precursor RNA, herein designated VGAM FOLDED PRECURSOR RNA, of VGAM270 are further described hereinbelow with reference to Table 1.

Nucleotide sequences of host target binding sites, such as BINDING SITE-I, BINDING SITE-II and BINDING SITE-III of FIG. 1, found on VGAM270 host target RNA, and schematic representation of the complementarity of each of these host target binding sites to VGAM270 RNA, herein designated VGAM RNA, are described hereinbelow with reference to Table 2.

As mentioned hereinabove with reference to FIG. 1, a function of VGAM270 gene, herein designated VGAM is inhibition of expression of VGAM270 target genes. It is appreciated that specific functions, and accordingly utilities, of VGAM270 correlate with, and may be deduced from, the identity of the target genes which VGAM270 binds and inhibits, and the function of these target genes, as elaborated hereinbelow.

PAIP2 (Accession NM_{—}016480) is a VGAM270 host target gene. PAIP2 BINDING SITE is HOST TARGET binding site found in the 3′ untranslated region of mRNA encoded by PAIP2, corresponding to a HOST TARGET binding site such as BINDING SITE I, BINDING SITE II or BINDING SITE III. Table 2 illustrates the complementarity of the nucleotide sequences of PAIP2 BINDING SITE, designated SEQ ID: 1687, to the nucleotide sequence of VGAM270 RNA, herein designated VGAM RNA, also designated SEQ ID:605.

A function of VGAM270 is therefore inhibition of PAIP2 (Accession NM_{—}016480). Accordingly, utilities of VGAM270 include diagnosis, prevention and treatment of diseases and clinical conditions associated with PAIP2. Protein (peptidyl-prolyl cis/trans isomerase) NIMA-interacting, 4 (parvulin) (PIN4, Accession NM_{—}006223) is another VGAM270 host target gene. PIN4 BINDING SITE is HOST TARGET binding site found in the 3′ untranslated region of mRNA encoded by PIN4, corresponding to a HOST TARGET binding site such as BINDING SITE I, BINDING SITE II or BINDING SITE III. Table 2 illustrates the complementarity of the nucleotide sequences of PIN4 BINDING SITE, designated SEQ ID:1282, to the nucleotide sequence of VGAM270 RNA, herein designated VGAM RNA, also designated SEQ ID:605.

Another function of VGAM270 is therefore inhibition of Protein (peptidyl-prolyl cis/trans isomerase) NIMA-interacting, 4 (parvulin) (PIN4, Accession NM_{—}006223), a gene which is a peptidyl-prolyl cis/trans isomerases. Accordingly, utilities of VGAM270 include diagnosis, prevention and treatment of diseases and clinical conditions associated with PIN4. The function of PIN4 has been established by previous studies. By searching an EST database using human PIN1 and E. coli parvulin sequences as probes, followed by screening a lung cDNA library, Uchida et al. (1999) isolated a cDNA encoding PIN4, which they called PAR14 (parvulin-14). Using a similar method, Rulten et al. (1999) isolated a cDNA encoding PIN4, which they called EPVH (eukaryotic parvulin homolog). Sequence analysis predicted that the 131-amino acid PIN4 protein contains a PPlase domain preceded by a 40-amino acid glycine- and lysine-rich N-terminal sequence. PIN4, however, lacks a WW domain, a nuclear localization motif, and residues required for phosphoprotein interactions. Multiple-tissue Northern blot analysis detected variable expression of an approximately 1.0-kb PIN4 transcript in all tissues tested, with notably lower expression in neuronal tissue. Transmission electron microscopy demonstrated preferential localization of PIN4 in the mitochondrial matrix. Functional analysis failed to show PPlase activity, possibly due to proteolytic degradation or different substrate requirements.

Full details of the abovementioned studies are described in the following publications, the disclosure of which are hereby incorporated by reference:
Rulten, S.; Thorpe, J.; Kay, J.: Identification of eukaryotic parvulin homologues: a new sub family of peptidylprolyl cis-trans isomerases. Biochem. Biophys. Res. Commun. 259: 557-562, 1999.; and
Uchida, T.; Fujimori, F.; Tradler, T.; Fischer, G.; Rahfeld, J.-U.: Identification and characterization of a 14 kDa human protein as a novel parvulin-like peptidyl prolyl cis/trans is.

Further studies establishing the function and utilities of PIN4 are found in John Hopkins OMIM database record ID 300252, and in sited publications numbered 1963 listed in the bibliography section hereinbelow, which are also hereby incorporated by reference. FIG. 1 further provides a conceptual description of a novel bioinformatically detected viral gene of the present invention, referred to here as Viral Genomic Address Messenger 271 (VGAM271) viral gene, which modulates expression of respective host target genes thereof, the function and utility of which host target genes is known in the art.

VGAM271 is a novel bioinformatically detected regulatory, non protein coding, viral micro RNA (miRNA) gene. The method by which VGAM271 was detected is described hereinabove with reference to FIGS. 1-8.

VGAM271 gene, herein designated VGAM GENE, is a viral gene contained in the genome of Vaccinia Virus. VGAM271 host target gene, herein designated VGAM HOST TARGET GENE, is a human gene contained in the human genome.

VGAM271 gene encodes a VGAM271 precursor RNA, herein designated VGAM PRECURSOR RNA. Similar to other miRNA genes, and unlike most ordinary genes, VGAM271 precursor RNA does not encode a protein. A nucleotide sequence identical or highly similar to the nucleotide sequence of VGAM271 precursor RNA is designated SEQ ID:257, and is provided hereinbelow with reference to the sequence listing part. Nucleotide sequence SEQ ID:257 is located at position 152876 relative to the genome of Vaccinia Virus.

VGAM271 precursor RNA folds onto itself, forming VGAM271 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM271 folded precursor RNA into VGAM271 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM271 RNA is designated SEQ ID:606, and is provided hereinbelow with reference to the sequence listing part.

VGAM271 host target gene, herein designated VGAM HOST TARGET GENE, encodes a corresponding messenger RNA, VGAM271 host target RNA, herein designated VGAM HOST TARGET RNA. VGAM271 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM271 RNA, herein designated VGAM RNA, binds complementarily to one or more host target binding sites located in untranslated regions of VGAM271 host target RNA, herein designated VGAM HOST TARGET RNA. This complementary binding is due to the fact that the nucleotide sequence of VGAM271 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM271 RNA, herein designated VGAM RNA, may have a different number of host target binding sites in untranslated regions of a VGAM271 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM271 RNA, herein designated VGAM RNA, to host target binding sites on VGAM271 host target RNA, herein designated VGAM HOST TARGET RNA, such as BINDING SITE I, BINDING SITE II and BINDING SITE III, inhibits translation of VGAM271 host target RNA into VGAM271 host target protein, herein designated VGAM HOST TARGET PROTEIN. VGAM host target protein is therefore outlined by a broken line.

It is appreciated that VGAM271 host target gene, herein designated VGAM HOST TARGET GENE, in fact represents a plurality of VGAM271 host target genes. The mRNA of each one of this plurality of VGAM271 host target genes comprises one or more host target binding sites, each having a nucleotide sequence which is at least partly complementary to VGAM271 RNA, herein designated VGAM RNA, and which when bound by VGAM271 RNA causes inhibition of translation of respective one or more VGAM271 host target proteins.

It is further appreciated by one skilled in the art that the mode of translational inhibition illustrated by FIG. 1 with specific reference to translational inhibition exerted by VGAM271 gene, herein designated VGAM GENE, on one or more VGAM271 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM271 is inhibition of expression of host target genes, as part of a novel viral mechanism of attacking a host. Accordingly, utilities of VGAM271 include diagnosis, prevention and treatment of viral infection by Vaccinia Virus. Specific functions, and accordingly utilities, of VGAM271 correlate with, and may be deduced from, the identity of the host target genes which VGAM271 binds and inhibits, and the function of these host target genes, as elaborated hereinbelow.

Nucleotide sequences of the VGAM271 precursor RNA, herein designated VGAM PRECURSOR RNA, and of the ‘diced’ VGAM271 RNA, herein designated VGAM RNA, and a schematic representation of the secondary folding of VGAM271 folded precursor RNA, herein designated VGAM FOLDED PRECURSOR RNA, of VGAM271 are further described hereinbelow with reference to Table 1.

Nucleotide sequences of host target binding sites, such as BINDING SITE-I, BINDING SITE-II and BINDING SITE-III of FIG. 1, found on VGAM271 host target RNA, and schematic representation of the complementarity of each of these host target binding sites to VGAM271 RNA, herein designated VGAM RNA, are described hereinbelow with reference to Table 2.

As mentioned hereinabove with reference to FIG. 1, a function of VGAM271 gene, herein designated VGAM is inhibition of expression of VGAM271 target genes. It is appreciated that specific functions, and accordingly utilities, of VGAM271 correlate with, and may be deduced from, the identity of the target genes which VGAM271 binds and inhibits, and the function of these target genes, as elaborated hereinbelow.

HLA-G Histocompatibility Antigen, Class I, G (HLA-G, Accession NM_{—}002127) is a VGAM271 host target gene. HLA-G BINDING SITE is HOST TARGET binding site found in the 3′ untranslated region of mRNA encoded by HLA-G, corresponding to a HOST TARGET binding site such as BINDING SITE I, BINDING SITE II or BINDING SITE III. Table 2 illustrates the complementarity of the nucleotide sequences of HLA-G BINDING SITE, designated SEQ ID:900, to the nucleotide sequence of VGAM271 RNA, herein designated VGAM RNA, also designated SEQ ID:606.

A function of VGAM271 is therefore inhibition of HLA-G Histocompatibility Antigen, Class I, G (HLA-G, Accession NM_{—}002127), a gene which involved in the presentation of foreign antigens to the immune system. Accordingly, utilities of VGAM271 include diagnosis, prevention and treatment of diseases and clinical conditions associated with HLA-G. The function of HLA-G has been established by previous studies. Extravillous trophoblast from normal human placenta and the BeWo adherent human choriocarcinoma cell line express an unusual form of class I HLA molecule. This molecule has an H chain of approximately 40 kD and is apparently nonpolymorphic. Ellis et al. (1990) isolated and sequenced a cDNA clone for this class I HLA antigen. The nucleotide sequence showed a high degree of homology with the published sequence of a genomic clone, HLA 6.0, which is the product of a class I locus other than A, B, or C. The new locus was provisionally designated HLAG. Using PCR, Ellis et al. (1990) demonstrated similar HLA class I sequences in cDNA from normal extravillous trophoblasts. Although there was some nucleotide sequence polymorphism, the amino acid sequence of the molecule was conserved; hence, it is unlikely to provoke immune responses even though it is found at the fetal-maternal interface. Considering the well established role of nonclassic HLA-G class I molecules in inhibiting natural killer cell function, the consequence of abnormal HLA-G expression in malignant cells should be the escape of tumors from immunosurveillance. To examine this hypothesis, Paul et al. (1998) analyzed HLA-G expression and NK sensitivity in human malignant melanoma cells. Two melanoma cell lines exhibited a high level of HLA-G transcription with differential HLA-G isoform transcription and protein expression patterns. A higher level of HLA-G transcription ex vivo was detected in a skin melanoma metastasis biopsy compared with a healthy skin fragment from the same individual. HLA-G protein isoforms other than membrane-bound HLA-G1 protected 1 melanoma cell line from NK lysis. It thus appeared of critical importance to consider the specific role of HLA-G expression in tumors in the design of cancer immunotherapies.

Full details of the abovementioned studies are described in the following publications, the disclosure of which are hereby incorporated by reference:
Ellis, S. A.; Palmer, M. S.; McMichael, A. J.: Human trophoblast and the choriocarcinoma cell line BeWo express a truncated HLA class I molecule. J. Immun. 144: 731-735, 1990.; and
Paul, P.; Rouas-Freiss, N.; Khalil-Daher, I.; Moreau, P.; Riteau, B.; Le Gal, F. A.; Avril, M. F.; Dausset, J.; Guillet, J. G.; Carosella, E. D.: HLA-G expression in melanoma: a way fo.

Further studies establishing the function and utilities of HLA-G are found in John Hopkins OMIM database record ID 142871, and in sited publications numbered 758, 2647-2656, 881, 2657-265 and 2662-2664 listed in the bibliography section hereinbelow, which are also hereby incorporated by reference. Histamine Receptor H2 (HRH2, Accession NM_{—}022304) is another VGAM271 host target gene. HRH2 BINDING SITE is HOST TARGET binding site found in the 5′ untranslated region of mRNA encoded by HRH2, corresponding to a HOST TARGET binding site such as BINDING SITE I, BINDING SITE II or BINDING SITE III. Table 2 illustrates the complementarity of the nucleotide sequences of HRH2 BINDING SITE, designated SEQ ID:1984, to the nucleotide sequence of VGAM271 RNA, herein designated VGAM RNA, also designated SEQ ID:606.

Another function of VGAM271 is therefore inhibition of Histamine Receptor H2 (HRH2, Accession NM_{—}022304), a gene which stimulates cAMP production and promotes a Th2 immune response. Accordingly, utilities of VGAM271 include diagnosis, prevention and treatment of diseases and clinical conditions associated with HRH2. The function of HRH2 has been established by previous studies. Members of the DEAD box protein family are putative ATP-dependent RNA helicases that have been implicated in pre-mRNA splicing and diverse other cellular functions. See RNA helicase 1 (HRH1; 600396). One sub family of the DEAD box family, the DEAH box proteins, appear to be somewhat specific to mRNA splicing. Several S. cerevisiae PRP (pre-mRNA processing) genes encode DEAH proteins. By RT-PCR of HeLa cell mRNA with degenerate primers based on conserved regions of DEAH box proteins, Ono et al. (1994) isolated partial cDNAs encoding 5 members of the human DEAH box family, including HRH1 and HRH2. They determined that HRH2 shares 61% amino acid sequence identity with S. cerevisiae JA1. Imamura et al. (1997) isolated cDNAs corresponding to the entire coding region of HRH2, which they called DBP1 (DEAH box protein 1). The predicted 813-amino acid protein contains 7 consecutive motifs characteristic of ATP-dependent RNA helicases, as well as consensus sequences for structural motifs of a DNA/RNA helicase with a DEAH box. Northern blot analysis revealed that DBP1 was expressed as a 3.4-kb mRNA in all tissues tested. An additional larger transcript was observed in many tissues. Gee et al. (1997) isolated cDNAs encoding the mouse HRH2 homolog, mDEAH9. Gee et al. (1997) reported that mDEAH9 and Prp43 are 65% identical over a 500-amino acid region spanning the central helicase domain and C-terminal region, and that mDEAH9 and HRH2 were 98% identical in the helicase domain. When expressed in yeast, mDEAH9 complemented the Prp43 mutation specifically, although with less efficiency than the native yeast protein. Immunofluorescence experiments showed that mDEAH9 colocalizes with splicing factor SC35 (OMIM Ref. No. 600813) in punctate nuclear speckles in mammalian cells, consistent with its predicted role as a pre-mRNA splicing factor. Gee et al. (1997) suggested that mDEAH9 represents a mammalian homolog of Prp43.

Full details of the abovementioned studies are described in the following publications, the disclosure of which are hereby incorporated by reference:
Imamura, O.; Sugawara, M.; Furuichi, Y.: Cloning and characterization of a putative human RNA helicase gene of the DEAH-box protein family. Biochem. Biophys. Res. Commun. 240: 335-340, 1997.; and
Ono, Y.; Ohno, M.; Shimura, Y.: Identification of a putative RNA helicase (HRH1), a human homolog of yeast Prp22. Molec. Cell. Biol. 14: 7611-7620, 1994.

Further studies establishing the function and utilities of HRH2 are found in John Hopkins OMIM database record ID 603403, and in sited publications numbered 1742-174 and 1624 listed in the bibliography section hereinbelow, which are also hereby incorporated by reference. Potassium Inwardly-rectifying Channel, Sub family J, Member 15 (KCNJ15, Accession NM_{—}002243) is another VGAM271 host target gene. KCNJ15 BINDING SITE is HOST TARGET binding site found in the 3′ untranslated region of mRNA encoded by KCNJ15, corresponding to a HOST TARGET binding site such as BINDING SITE I, BINDING SITE II or BINDING SITE III. Table 2 illustrates the complementarity of the nucleotide sequences of KCNJ15 BINDING SITE, designated SEQ ID:916, to the nucleotide sequence of VGAM271 RNA, herein designated VGAM RNA, also designated SEQ ID:606.

Another function of VGAM271 is therefore inhibition of Potassium Inwardly-rectifying Channel, Sub family J, Member 15 (KCNJ15, Accession NM_{—}002243). Accordingly, utilities of VGAM271 include diagnosis, prevention and treatment of diseases and clinical conditions associated with KCNJ15. Chromosome 1 Open Reading Frame 8 (Clorf8, Accession NM_{—}004872) is another VGAM271 host target gene. Clorf8 BINDING SITE is HOST TARGET binding site found in the 5′ untranslated region of mRNA encoded by Clorf8, corresponding to a HOST TARGET binding site such as BINDING SITE I, BINDING SITE II or BINDING SITE III. Table 2 illustrates the complementarity of the nucleotide sequences of Clorf8 BINDING SITE, designated SEQ ID: 1161, to the nucleotide sequence of VGAM271 RNA, herein designated VGAM RNA, also designated SEQ ID:606.

Another function of VGAM271 is therefore inhibition of Chromosome 1 Open Reading Frame 8 (Clorf8, Accession NM_{—}004872). Accordingly, utilities of VGAM271 include diagnosis, prevention and treatment of diseases and clinical conditions associated with Clorf8. FLJ10738 (Accession NM_{—}018199) is another VGAM271 host target gene. FLJ10738 BINDING SITE is HOST TARGET binding site found in the 3′ untranslated region of mRNA encoded by FLJ10738, corresponding to a HOST TARGET binding site such as BINDING SITE I, BINDING SITE II or BINDING SITE III. Table 2 illustrates the complementarity of the nucleotide sequences of FLJ10738 BINDING SITE, designated SEQ ID: 1798, to the nucleotide sequence of VGAM271 RNA, herein designated VGAM RNA, also designated SEQ ID:606.

Another function of VGAM271 is therefore inhibition of FLJ10738 (Accession NM_{—}018199). Accordingly, utilities of VGAM271 include diagnosis, prevention and treatment of diseases and clinical conditions associated with FLJ10738. FLJ23511 (Accession NM_{—}032239) is another VGAM271 host target gene. FLJ23511 BINDING SITE is HOST TARGET binding site found in the 3′ untranslated region of mRNA encoded by FLJ23511, corresponding to a HOST TARGET binding site such as BINDING SITE I, BINDING SITE II or BINDING SITE III. Table 2 illustrates the complementarity of the nucleotide sequences of FLJ23511 BINDING SITE, designated SEQ ID:2239, to the nucleotide sequence of VGAM271 RNA, herein designated VGAM RNA, also designated SEQ ID:606.

Another function of VGAM271 is therefore inhibition of FLJ23511 (Accession NM_{—}032239). Accordingly, utilities of VGAM271 include diagnosis, prevention and treatment of diseases and clinical conditions associated with FLJ23511. GRB2-associated Binding Protein 3 (GAB3, Accession NM_{—}080612) is another VGAM271 host target gene. GAB3 BINDING SITE is HOST TARGET binding site found in the 3′ untranslated region of mRNA encoded by GAB3, corresponding to a HOST TARGET binding site such as BINDING SITE I, BINDING SITE II or BINDING SITE III. Table 2 illustrates the complementarity of the nucleotide sequences of GAB3 BINDING SITE, designated SEQ ID:2377, to the nucleotide sequence of VGAM271 RNA, herein designated VGAM RNA, also designated SEQ ID:606.

Another function of VGAM271 is therefore inhibition of GRB2-associated Binding Protein 3 (GAB3, Accession NM_{—}080612). Accordingly, utilities of VGAM271 include diagnosis, prevention and treatment of diseases and clinical conditions associated with GAB3. Inositol 1,3,4-triphosphate 5/6 Kinase (ITPK1, Accession NM_{—}014216) is another VGAM271 host target gene. ITPK1 BINDING SITE is HOST TARGET binding site found in the 3′ untranslated region of mRNA encoded by ITPK1, corresponding to a HOST TARGET binding site such as BINDING SITE I, BINDING SITE II or BINDING SITE III. Table 2 illustrates the complementarity of the nucleotide sequences of ITPK1 BINDING SITE, designated SEQ ID: 1484, to the nucleotide sequence of VGAM271 RNA, herein designated VGAM RNA, also designated SEQ ID:606.

Another function of VGAM271 is therefore inhibition of Inositol 1,3,4-triphosphate 5/6 Kinase (ITPK1, Accession NM_{—}014216). Accordingly, utilities of VGAM271 include diagnosis, prevention and treatment of diseases and clinical conditions associated with ITPK1. TRIP-Br2 (Accession NM_{—}014755) is another VGAM271 host target gene. TRIP-Br2 BINDING SITE is HOST TARGET binding site found in the 3′ untranslated region of mRNA encoded by TRIP-Br2, corresponding to a HOST TARGET binding site such as BINDING SITE I, BINDING SITE II or BINDING SITE III. Table 2 illustrates the complementarity of the nucleotide sequences of TRIP-Br2 BINDING SITE, designated SEQ ID:1539, to the nucleotide sequence of VGAM271 RNA, herein designated VGAM RNA, also designated SEQ ID:606.

Another function of VGAM271 is therefore inhibition of TRIP-Br2 (Accession NM_{—}014755). Accordingly, utilities of VGAM271 include diagnosis, prevention and treatment of diseases and clinical conditions associated with TRIP-Br2. LOC152313 (Accession XM_{—}098190) is another VGAM271 host target gene. LOC152313 BINDING SITE is HOST TARGET binding site found in the 3′ untranslated region of mRNA encoded by LOC152313, corresponding to a HOST TARGET binding site such as BINDING SITE I, BINDING SITE II or BINDING SITE III. Table 2 illustrates the complementarity of the nucleotide sequences of LOC152313 BINDING SITE, designated SEQ ID:3355, to the nucleotide sequence of VGAM271 RNA, herein designated VGAM RNA, also designated SEQ ID:606.

Another function of VGAM271 is therefore inhibition of LOC152313 (Accession XM_{—}098190). Accordingly, utilities of VGAM271 include diagnosis, prevention and treatment of diseases and clinical conditions associated with LOC152313. LOC256642 (Accession XM_{—}172797) is another VGAM271 host target gene. LOC256642 BINDING SITE is HOST TARGET binding site found in the 3′ untranslated region of mRNA encoded by LOC256642, corresponding to a HOST TARGET binding site such as BINDING SITE I, BINDING SITE II or BINDING SITE III. Table 2 illustrates the complementarity of the nucleotide sequences of LOC256642 BINDING SITE, designated SEQ ID:3701, to the nucleotide sequence of VGAM271 RNA, herein designated VGAM RNA, also designated SEQ ID:606.

Another function of VGAM271 is therefore inhibition of LOC256642 (Accession XM_{—}172797). Accordingly, utilities of VGAM271 include diagnosis, prevention and treatment of diseases and clinical conditions associated with LOC256642. FIG. 1 further provides a conceptual description of a novel bioinformatically detected viral gene of the present invention, referred to here as Viral Genomic Address Messenger 272 (VGAM272) viral gene, which modulates expression of respective host target genes thereof, the function and utility of which host target genes is known in the art.

VGAM272 is a novel bioinformatically detected regulatory, non protein coding, viral micro RNA (miRNA) gene. The method by which VGAM272 was detected is described hereinabove with reference to FIGS. 1-8.

VGAM272 gene, herein designated VGAM GENE, is a viral gene contained in the genome of Vaccinia Virus. VGAM272 host target gene, herein designated VGAM HOST TARGET GENE, is a human gene contained in the human genome.

VGAM272 gene encodes a VGAM272 precursor RNA, herein designated VGAM PRECURSOR RNA. Similar to other miRNA genes, and unlike most ordinary genes, VGAM272 precursor RNA does not encode a protein. A nucleotide sequence identical or highly similar to the nucleotide sequence of VGAM272 precursor RNA is designated SEQ ID:258, and is provided hereinbelow with reference to the sequence listing part. Nucleotide sequence SEQ ID:258 is located at position 154919 relative to the genome of Vaccinia Virus.

VGAM272 precursor RNA folds onto itself, forming VGAM272 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM272 folded precursor RNA into VGAM272 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM272 RNA is designated SEQ ID:607, and is provided hereinbelow with reference to the sequence listing part.

VGAM272 host target gene, herein designated VGAM HOST TARGET GENE, encodes a corresponding messenger RNA, VGAM272 host target RNA, herein designated VGAM HOST TARGET RNA. VGAM272 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM272 RNA, herein designated VGAM RNA, binds complementarily to one or more host target binding sites located in untranslated regions of VGAM272 host target RNA, herein designated VGAM HOST TARGET RNA. This complementary binding is due to the fact that the nucleotide sequence of VGAM272 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM272 RNA, herein designated VGAM RNA, may have a different number of host target binding sites in untranslated regions of a VGAM272 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM272 RNA, herein designated VGAM RNA, to host target binding sites on VGAM272 host target RNA, herein designated VGAM HOST TARGET RNA, such as BINDING SITE I, BINDING SITE II and BINDING SITE III, inhibits translation of VGAM272 host target RNA into VGAM272 host target protein, herein designated VGAM HOST TARGET PROTEIN. VGAM host target protein is therefore outlined by a broken line.

It is appreciated that VGAM272 host target gene, herein designated VGAM HOST TARGET GENE, in fact represents a plurality of VGAM272 host target genes. The mRNA of each one of this plurality of VGAM272 host target genes comprises one or more host target binding sites, each having a nucleotide sequence which is at least partly complementary to VGAM272 RNA, herein designated VGAM RNA, and which when bound by VGAM272 RNA causes inhibition of translation of respective one or more VGAM272 host target proteins.

It is further appreciated by one skilled in the art that the mode of translational inhibition illustrated by FIG. 1 with specific reference to translational inhibition exerted by VGAM272 gene, herein designated VGAM GENE, on one or more VGAM272 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM272 is inhibition of expression of host target genes, as part of a novel viral mechanism of attacking a host. Accordingly, utilities of VGAM272 include diagnosis, prevention and treatment of viral infection by Vaccinia Virus. Specific functions, and accordingly utilities, of VGAM272 correlate with, and may be deduced from, the identity of the host target genes which VGAM272 binds and inhibits, and the function of these host target genes, as elaborated hereinbelow.

Nucleotide sequences of the VGAM272 precursor RNA, herein designated VGAM PRECURSOR RNA, and of the ‘diced’ VGAM272 RNA, herein designated VGAM RNA, and a schematic representation of the secondary folding of VGAM272 folded precursor RNA, herein designated VGAM FOLDED PRECURSOR RNA, of VGAM272 are further described hereinbelow with reference to Table 1.

Nucleotide sequences of host target binding sites, such as BINDING SITE-I, BINDING SITE-II and BINDING SITE-III of FIG. 1, found on VGAM272 host target RNA, and schematic representation of the complementarity of each of these host target binding sites to VGAM272 RNA, herein designated VGAM RNA, are described hereinbelow with reference to Table 2.

As mentioned hereinabove with reference to FIG. 1, a function of VGAM272 gene, herein designated VGAM is inhibition of expression of VGAM272 target genes. It is appreciated that specific functions, and accordingly utilities, of VGAM272 correlate with, and may be deduced from, the identity of the target genes which VGAM272 binds and inhibits, and the function of these target genes, as elaborated hereinbelow.

Chromosome 22 Open Reading Frame 2 (C22orf2, Accession XM_{—}170492) is a VGAM272 host target gene. C22orf2 BINDING SITE is HOST TARGET binding site found in the 3′ untranslated region of mRNA encoded by C22orf2, corresponding to a HOST TARGET binding site such as BINDING SITE I, BINDING SITE II or BINDING SITE III. Table 2 illustrates the complementarity of the nucleotide sequences of C22orf2 BINDING SITE, designated SEQ ID:3655, to the nucleotide sequence of VGAM272 RNA, herein designated VGAM RNA, also designated SEQ ID:607.

A function of VGAM272 is therefore inhibition of Chromosome 22 Open Reading Frame 2 (C22orf2, Accession XM_{—}170492). Accordingly, utilities of VGAM272 include diagnosis, prevention and treatment of diseases and clinical conditions associated with C22orf2. LOC153277 (Accession XM_{—}098346) is another VGAM272 host target gene. LOC153277 BINDING SITE is HOST TARGET binding site found in the 3′ untranslated region of mRNA encoded by LOC153277, corresponding to a HOST TARGET binding site such as BINDING SITE I, BINDING SITE II or BINDING SITE III. Table 2 illustrates the complementarity of the nucleotide sequences of LOC153277 BINDING SITE, designated SEQ ID:3369, to the nucleotide sequence of VGAM272 RNA, herein designated VGAM RNA, also designated SEQ ID:607.

Another function of VGAM272 is therefore inhibition of LOC153277 (Accession XM_{—}098346). Accordingly, utilities of VGAM272 include diagnosis, prevention and treatment of diseases and clinical conditions associated with LOC153277. FIG. 1 further provides a conceptual description of a novel bioinformatically detected viral gene of the present invention, referred to here as Viral Genomic Address Messenger 273 (VGAM273) viral gene, which modulates expression of respective host target genes thereof, the function and utility of which host target genes is known in the art.

VGAM273 is a novel bioinformatically detected regulatory, non protein coding, viral micro RNA (miRNA) gene. The method by which VGAM273 was detected is described hereinabove with reference to FIGS. 1-8.

VGAM273 gene, herein designated VGAM GENE, is a viral gene contained in the genome of Vaccinia Virus. VGAM273 host target gene, herein designated VGAM HOST TARGET GENE, is a human gene contained in the human genome.

VGAM273 gene encodes a VGAM273 precursor RNA, herein designated VGAM PRECURSOR RNA. Similar to other miRNA genes, and unlike most ordinary genes, VGAM273 precursor RNA does not encode a protein. A nucleotide sequence identical or highly similar to the nucleotide sequence of VGAM273 precursor RNA is designated SEQ ID:259, and is provided hereinbelow with reference to the sequence listing part. Nucleotide sequence SEQ ID:259 is located at position 154466 relative to the genome of Vaccinia Virus.

VGAM273 precursor RNA folds onto itself, forming VGAM273 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM273 folded precursor RNA into VGAM273 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 91%) nucleotide sequence of VGAM273 RNA is designated SEQ ID:608, and is provided hereinbelow with reference to the sequence listing part.

VGAM273 host target gene, herein designated VGAM HOST TARGET GENE, encodes a corresponding messenger RNA, VGAM273 host target RNA, herein designated VGAM HOST TARGET RNA. VGAM273 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM273 RNA, herein designated VGAM RNA, binds complementarily to one or more host target binding sites located in untranslated regions of VGAM273 host target RNA, herein designated VGAM HOST TARGET RNA. This complementary binding is due to the fact that the nucleotide sequence of VGAM273 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM273 RNA, herein designated VGAM RNA, may have a different number of host target binding sites in untranslated regions of a VGAM273 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM273 RNA, herein designated VGAM RNA, to host target binding sites on VGAM273 host target RNA, herein designated VGAM HOST TARGET RNA, such as BINDING SITE I, BINDING SITE II and BINDING SITE III, inhibits translation of VGAM273 host target RNA into VGAM273 host target protein, herein designated VGAM HOST TARGET PROTEIN. VGAM host target protein is therefore outlined by a broken line.

It is appreciated that VGAM273 host target gene, herein designated VGAM HOST TARGET GENE, in fact represents a plurality of VGAM273 host target genes. The mRNA of each one of this plurality of VGAM273 host target genes comprises one or more host target binding sites, each having a nucleotide sequence which is at least partly complementary to VGAM273 RNA, herein designated VGAM RNA, and which when bound by VGAM273 RNA causes inhibition of translation of respective one or more VGAM273 host target proteins.

It is further appreciated by one skilled in the art that the mode of translational inhibition illustrated by FIG. 1 with specific reference to translational inhibition exerted by VGAM273 gene, herein designated VGAM GENE, on one or more VGAM273 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM273 is inhibition of expression of host target genes, as part of a novel viral mechanism of attacking a host. Accordingly, utilities of VGAM273 include diagnosis, prevention and treatment of viral infection by Vaccinia Virus. Specific functions, and accordingly utilities, of VGAM273 correlate with, and may be deduced from, the identity of the host target genes which VGAM273 binds and inhibits, and the function of these host target genes, as elaborated hereinbelow.

Nucleotide sequences of the VGAM273 precursor RNA, herein designated VGAM PRECURSOR RNA, and of the ‘diced’ VGAM273 RNA, herein designated VGAM RNA, and a schematic representation of the secondary folding of VGAM273 folded precursor RNA, herein designated VGAM FOLDED PRECURSOR RNA, of VGAM273 are further described hereinbelow with reference to Table 1.

Nucleotide sequences of host target binding sites, such as BINDING SITE-I, BINDING SITE-II and BINDING SITE-III of FIG. 1, found on VGAM273 host target RNA, and schematic representation of the complementarity of each of these host target binding sites to VGAM273 RNA, herein designated VGAM RNA, are described hereinbelow with reference to Table 2.

As mentioned hereinabove with reference to FIG. 1, a function of VGAM273 gene, herein designated VGAM is inhibition of expression of VGAM273 target genes. It is appreciated that specific functions, and accordingly utilities, of VGAM273 correlate with, and may be deduced from, the identity of the target genes which VGAM273 binds and inhibits, and the function of these target genes, as elaborated hereinbelow.

SH3-domain Binding Protein 4 (SH3BP4, Accession NM_{—}014521) is a VGAM273 host target gene. SH3BP4 BINDING SITE is HOST TARGET binding site found in the 3′ untranslated region of mRNA encoded by SH3BP4, corresponding to a HOST TARGET binding site such as BINDING SITE I, BINDING SITE II or BINDING SITE III. Table 2 illustrates the complementarity of the nucleotide sequences of SH3BP4 BINDING SITE, designated SEQ ID:1506, to the nucleotide sequence of VGAM273 RNA, herein designated VGAM RNA, also designated SEQ ID:608.

A function of VGAM273 is therefore inhibition of SH3-domain Binding Protein 4 (SH3BP4, Accession NM_{—}014521), a gene which is of unknown function, contains SH3-domain binding protein 4; similar to the EH-binding protein. Accordingly, utilities of VGAM273 include diagnosis, prevention and treatment of diseases and clinical conditions associated with SH3BP4. The function of SH3BP4 has been established by previous studies. A major element of the cornea is a transparent stroma produced and maintained by corneal fibroblasts, or keratocytes. Using differential display of RNA from normal and macular corneal dystrophy cultured keratocytes, followed by screening a corneal fibroblast library, Dunlevy et al. (1999) identified a cDNA encoding SH3BP4. The deduced 963-amino acid SH3BP4 protein contains 3 asn-pro-phe (NPF) motifs, which are EPS15 (OMIM Ref. No. 600051) homology (EH)-binding sites (see OMIM Ref. No. NUMB; 603728); an SH3 domain; a PXXP motif; a bipartite nuclear targeting signal; and a tyrosine phosphorylation site. Sequence analysis predicted that SH3BP4 is identical to a 479-amino acid EH-binding protein (Wong et al., 1995) except for the presence of an additional 73 N-terminal and 411 mid- to C-terminal residues in SH3BP4. Northern blot analysis revealed ubiquitous expression of a 5.6-kb transcript, with highest levels in pancreas, low levels in kidney, skeletal muscle, and liver, and lowest levels in lung and brain. Expression was also detected in cultured normal keratocytes. Using FISH, Dunlevy et al. (1999) mapped the SH3BP4 gene to 2q37.1-q37.2.

Full details of the abovementioned studies are described in the following publications, the disclosure of which are hereby incorporated by reference:
Dunlevy, J. R.; Berryhill, B. L.; Vergnes, J.-P.; Sundar Raj, N.; Hassell, J. R.: Cloning, chromosomal localization, and characterization of cDNA from a novel gene, SH3BP4, expressed by human corneal fibroblasts. Genomics 62: 519-524, 1999.; and
Wong, W. T.; Schumacher, C.; Salcini, A. E.; Romano, A.; Castagnino, P.; Pelicci, P. G.; DiFiore, P. P.: A protein-binding domain, EH, identified in the receptor tyrosine kinase substrat.

Further studies establishing the function and utilities of SH3BP4 are found in John Hopkins OMIM database record ID 605611, and in sited publications numbered 1565-1566 listed in the bibliography section hereinbelow, which are also hereby incorporated by reference. Ubiquitin Fusion Degradation 1-like (UFD1L, Accession XM_{—}055490) is another VGAM273 host target gene. UFD1L BINDING SITE is HOST TARGET binding site found in the 3′ untranslated region of mRNA encoded by UFD1L, corresponding to a HOST TARGET binding site such as BINDING SITE I, BINDING SITE II or BINDING SITE III. Table 2 illustrates the complementarity of the nucleotide sequences of UFD1L BINDING SITE, designated SEQ ID:2973, to the nucleotide sequence of VGAM273 RNA, herein designated VGAM RNA, also designated SEQ ID:608.

Another function of VGAM273 is therefore inhibition of Ubiquitin Fusion Degradation 1-like (UFD1L, Accession XM_{—}055490), a gene which is essential component of the ubiquitin-dependent proteolytic pathway. Accordingly, utilities of VGAM273 include diagnosis, prevention and treatment of diseases and clinical conditions associated with UFD1L. The function of UFD1L has been established by previous studies. In a search for genes in the 22q11.2 region possibly implicated in the DiGeorge syndrome (OMIM Ref. No. 188400), Pizutti et al. (1997) identified a gene whose functional features and tissue-specific expression suggested a distinct role in embryogenesis. Symbolized UFD1L by them (for ubiquitin fusion degradation 1-like), the gene encodes the human homolog of the yeast ubiquitin fusion degradation 1 (UFD1) protein that is involved in the degradation of ubiquitin fusion proteins (see OMIM Ref. No. 191320). Cloning and characterization of the murine homolog (Ufd11) showed it to be expressed during embryogenesis in the eyes and in the inner ear primordia. These findings suggested to Pizutti et al. (1997) that the proteolytic pathway recognizing ubiquitin fusion proteins for degradation is conserved in vertebrates and that UFD1L gene hemizygosity may be the cause of some of the CATCH22-associated developmental defects. The basic helix-loop-helix transcription factor dHAND (HAND2; 602407) is required for survival of cells in the neural crest-derived branchial and aortic arch arteries and the right ventricle. Mice lacking endothelin-1 (EDN1; 131240) have cardiac and cranial neural crest defects typical of the 22q11 deletion syndrome and display downregulation of dHAND, suggesting that a molecular pathway involving dHAND may be disrupted in that syndrome. The HAND2, EDN1, and ET1 receptor (EDNRA; 131243) genes do not map to 22q11, the DiGeorge syndrome critical region, in humans. In a screen for mouse genes dependent on dHAND, Yamagishi et al. (1999) identified Ufd1, which maps to human 22q11 and encodes a protein involved in degradation of ubiquitinated proteins. Mouse Ufd1 was specifically expressed in most tissues affected in patients with the DiGeorge (22q11 deletion) syndrome. Yamagishi et al. (1999) found, furthermore, that the human UFD1L gene was deleted in all 182 patients studied with the 22q11 deletion, and a smaller deletion of approximately 20 kb that removed exons 1 to 3 of UFD1L was found in 1 individual with features typical of 22q11 deletion syndrome. In the individual with the smaller deletion, patient J.F., Yamagishi et al. (1999) found that the CDC45L gene (OMIM Ref. No. 603465), which is immediately telomeric of UFD1L, was the site of the deletion in the region between exons 5 and 6 of the 5-prime breakpoint. They considered that the deletion in CDC45L may act as a modifier of the phenotype in patient J.F. UFD1L and CDC45L are transcribed in opposite directions. The deletion left exons 4 to 12 of UFD1L intact; the first 5 exons of CDC45L were deleted. Patient J.F. had nearly all of the features commonly associated with the 2-Mb 22q11 deletion. Four days after birth the patient was diagnosed with interrupted aortic arch, persistent truncus arteriosus, cleft palate, small mouth, low-set ears, broad nasal bridge, neonatal hypocalcemia, T-lymphocyte deficiency, and syndactyly of her toes. The deletion was not present in her parents or in 100 control subjects.

Full details of the abovementioned studies are described in the following publications, the disclosure of which are hereby incorporated by reference:
Pizutti, A.; Novelli, G.; Ratti, A.; Amati, F.; Mari, A.; Calabrese, G.; Nicolis, S.; Silani, V.; Marino, B.; Scarlato, G.; Ottolenghi, S.; Dallapiccola, B.: UFD1L, a developmentally expressed ubiquitination gene, is deleted in CATCH 22 syndrome. Hum. Molec. Genet. 6: 259-265, 1997.; and
Yamagishi, H.; Garg, V.; Matsuoka, R.; Thomas, T.; Srivastava, D.: A molecular pathway revealing a genetic basis for human cardiac and craniofacial defects. Science 283: 1158-1161, 199.

Further studies establishing the function and utilities of UFD1L are found in John Hopkins OMIM database record ID 601754, and in sited publications numbered 1446-1447 listed in the bibliography section hereinbelow, which are also hereby incorporated by reference. FLJ20081 (Accession NM_{—}017658) is another VGAM273 host target gene. FLJ20081 BINDING SITE is HOST TARGET binding site found in the 3′ untranslated region of mRNA encoded by FLJ20081, corresponding to a HOST TARGET binding site such as BINDING SITE I, BINDING SITE II or BINDING SITE III. Table 2 illustrates the complementarity of the nucleotide sequences of FLJ20081 BINDING SITE, designated SEQ ID: 1736, to the nucleotide sequence of VGAM273 RNA, herein designated VGAM RNA, also designated SEQ ID:608.

Another function of VGAM273 is therefore inhibition of FLJ20081 (Accession NM_{—}017658). Accordingly, utilities of VGAM273 include diagnosis, prevention and treatment of diseases and clinical conditions associated with FLJ20081. KIAA1635 (Accession XM_{—}042203) is another VGAM273 host target gene. KIAA1635 BINDING SITE is HOST TARGET binding site found in the 3′ untranslated region of mRNA encoded by KIAA1635, corresponding to a HOST TARGET binding site such as BINDING SITE I, BINDING SITE II or BINDING SITE III. Table 2 illustrates the complementarity of the nucleotide sequences of KIAA1635 BINDING SITE, designated SEQ ID:2804, to the nucleotide sequence of VGAM273 RNA, herein designated VGAM RNA, also designated SEQ ID:608.

Another function of VGAM273 is therefore inhibition of KIAA1635 (Accession XM_{—}042203). Accordingly, utilities of VGAM273 include diagnosis, prevention and treatment of diseases and clinical conditions associated with KIAA1635. PRO1257 (Accession NM_{—}018578) is another VGAM273 host target gene. PRO1257 BINDING SITE is HOST TARGET binding site found in the 5′ untranslated region of mRNA encoded by PRO1257, corresponding to a HOST TARGET binding site such as BINDING SITE I, BINDING SITE II or BINDING SITE III. Table 2 illustrates the complementarity of the nucleotide sequences of PRO1257 BINDING SITE, designated SEQ ID:1848, to the nucleotide sequence of VGAM273 RNA, herein designated VGAM RNA, also designated SEQ ID:608.

Another function of VGAM273 is therefore inhibition of PRO1257 (Accession NM_{—}018578). Accordingly, utilities of VGAM273 include diagnosis, prevention and treatment of diseases and clinical conditions associated with PRO1257. LOC153020 (Accession XM_{—}087578) is another VGAM273 host target gene. LOC153020 BINDING SITE is HOST TARGET binding site found in the 3′ untranslated region of mRNA encoded by LOC153020, corresponding to a HOST TARGET binding site such as BINDING SITE I, BINDING SITE II or BINDING SITE III. Table 2 illustrates the complementarity of the nucleotide sequences of LOC153020 BINDING SITE, designated SEQ ID:3177, to the nucleotide sequence of VGAM273 RNA, herein designated VGAM RNA, also designated SEQ ID:608.

Another function of VGAM273 is therefore inhibition of LOC153020 (Accession XM_{—}087578). Accordingly, utilities of VGAM273 include diagnosis, prevention and treatment of diseases and clinical conditions associated with LOC153020. FIG. 1 further provides a conceptual description of a novel bioinformatically detected viral gene of the present invention, referred to here as Viral Genomic Address Messenger 274 (VGAM274) viral gene, which modulates expression of respective host target genes thereof, the function and utility of which host target genes is known in the art.

VGAM274 is a novel bioinformatically detected regulatory, non protein coding, viral micro RNA (miRNA) gene. The method by which VGAM274 was detected is described hereinabove with reference to FIGS. 1-8.

VGAM274 gene, herein designated VGAM GENE, is a viral gene contained in the genome of Vaccinia Virus. VGAM274 host target gene, herein designated VGAM HOST TARGET GENE, is a human gene contained in the human genome.

VGAM274 gene encodes a VGAM274 precursor RNA, herein designated VGAM PRECURSOR RNA. Similar to other miRNA genes, and unlike most ordinary genes, VGAM274 precursor RNA does not encode a protein. A nucleotide sequence identical or highly similar to the nucleotide sequence of VGAM274 precursor RNA is designated SEQ ID:260, and is provided hereinbelow with reference to the sequence listing part. Nucleotide sequence SEQ ID:260 is located at position 156944 relative to the genome of Vaccinia Virus.

VGAM274 precursor RNA folds onto itself, forming VGAM274 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM274 folded precursor RNA into VGAM274 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM274 RNA is designated SEQ ID:609, and is provided hereinbelow with reference to the sequence listing part.

VGAM274 host target gene, herein designated VGAM HOST TARGET GENE, encodes a corresponding messenger RNA, VGAM274 host target RNA, herein designated VGAM HOST TARGET RNA. VGAM274 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM274 RNA, herein designated VGAM RNA, binds complementarily to one or more host target binding sites located in untranslated regions of VGAM274 host target RNA, herein designated VGAM HOST TARGET RNA. This complementary binding is due to the fact that the nucleotide sequence of VGAM274 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM274 RNA, herein designated VGAM RNA, may have a different number of host target binding sites in untranslated regions of a VGAM274 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM274 RNA, herein designated VGAM RNA, to host target binding sites on VGAM274 host target RNA, herein designated VGAM HOST TARGET RNA, such as BINDING SITE I, BINDING SITE II and BINDING SITE III, inhibits translation of VGAM274 host target RNA into VGAM274 host target protein, herein designated VGAM HOST TARGET PROTEIN. VGAM host target protein is therefore outlined by a broken line.

It is appreciated that VGAM274 host target gene, herein designated VGAM HOST TARGET GENE, in fact represents a plurality of VGAM274 host target genes. The mRNA of each one of this plurality of VGAM274 host target genes comprises one or more host target binding sites, each having a nucleotide sequence which is at least partly complementary to VGAM274 RNA, herein designated VGAM RNA, and which when bound by VGAM274 RNA causes inhibition of translation of respective one or more VGAM274 host target proteins.

It is further appreciated by one skilled in the art that the mode of translational inhibition illustrated by FIG. 1 with specific reference to translational inhibition exerted by VGAM274 gene, herein designated VGAM GENE, on one or more VGAM274 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM274 is inhibition of expression of host target genes, as part of a novel viral mechanism of attacking a host. Accordingly, utilities of VGAM274 include diagnosis, prevention and treatment of viral infection by Vaccinia Virus. Specific functions, and accordingly utilities, of VGAM274 correlate with, and may be deduced from, the identity of the host target genes which VGAM274 binds and inhibits, and the function of these host target genes, as elaborated hereinbelow.

Nucleotide sequences of the VGAM274 precursor RNA, herein designated VGAM PRECURSOR RNA, and of the ‘diced’ VGAM274 RNA, herein designated VGAM RNA, and a schematic representation of the secondary folding of VGAM274 folded precursor RNA, herein designated VGAM FOLDED PRECURSOR RNA, of VGAM274 are further described hereinbelow with reference to Table 1.

Nucleotide sequences of host target binding sites, such as BINDING SITE-I, BINDING SITE-II and BINDING SITE-III of FIG. 1, found on VGAM274 host target RNA, and schematic representation of the complementarity of each of these host target binding sites to VGAM274 RNA, herein designated VGAM RNA, are described hereinbelow with reference to Table 2.

As mentioned hereinabove with reference to FIG. 1, a function of VGAM274 gene, herein designated VGAM is inhibition of expression of VGAM274 target genes. It is appreciated that specific functions, and accordingly utilities, of VGAM274 correlate with, and may be deduced from, the identity of the target genes which VGAM274 binds and inhibits, and the function of these target genes, as elaborated hereinbelow.

Basonuclin (BNC, Accession NM_{—}001717) is a VGAM274 host target gene. BNC BINDING SITE is HOST TARGET binding site found in the 3′ untranslated region of mRNA encoded by BNC, corresponding to a HOST TARGET binding site such as BINDING SITE I, BINDING SITE II or BINDING SITE III. Table 2 illustrates the complementarity of the nucleotide sequences of BNC BINDING SITE, designated SEQ ID:851, to the nucleotide sequence of VGAM274 RNA, herein designated VGAM RNA, also designated SEQ ID:609.

A function of VGAM274 is therefore inhibition of Basonuclin (BNC, Accession NM_{—}001717), a gene which plays a role in the maintenance of proliferative capacity and prevention of terminal differentiation of keratinocytes. Accordingly, utilities of VGAM274 include diagnosis, prevention and treatment of diseases and clinical conditions associated with BNC. The function of BNC has been established by previous studies. Basonuclin (BNC) is a protein found mainly in cells of the basal layer of stratified squamous epithelia. Tseng and Green (1992) isolated a cDNA encoding this protein from mRNA of cultural human keratinocytes. The basonuclin cDNA encodes a 993-amino acid polypeptide that is located in the nucleus and contains 6 zinc finger motifs of the C2H2 class, as in known transcription factors. Basonuclin is expressed in cells that are able to undergo division but are not necessarily in the division cycle; the protein is not found in terminally differentiated cells (Tseng and Green, 1994). These properties suggested that basonuclin performs a transcriptional regulatory function related to promotion of keratinocyte growth or suppression of keratinocyte differentiation. Teumer et al. (1997) cloned and sequenced the basonuclin gene from a human genomic library. By analysis of human/rodent hybrid cells, they mapped it to chromosome 15. The transcription unit spans nearly 29 kb of sequence. The coding region is distributed over 5 exons and the 3 pairs of zinc fingers encoded by the last 2 exons. The 5-prime flanking sequence and first exon are unusually rich in GC content and in CpG dinucleotides. This sequence region contains numerous binding sites for the transcription factor Sp1 (OMIM Ref. No. 189906).

Full details of the abovementioned studies are described in the following publications, the disclosure of which are hereby incorporated by reference:
Tseng, H.; Green, H.: Basonuclin: a keratinocyte protein with multiple paired zinc fingers. Proc. Nat. Acad. Sci. 89: 10311-10315, 1992.; and
Tseng, H.; Green, H.: Association of basonuclin with ability of keratinocytes to multiply and with absence of terminal differentiation. J. Cell Biol. 126: 495-506, 1994.

Further studies establishing the function and utilities of BNC are found in John Hopkins OMIM database record ID 601930, and in sited publications numbered 1448-1450 listed in the bibliography section hereinbelow, which are also hereby incorporated by reference. Protein S (alpha) (PROS1, Accession XM_{—}113400) is another VGAM274 host target gene. PROS1 BINDING SITE is HOST TARGET binding site found in the 5′ untranslated region of mRNA encoded by PROS1, corresponding to a HOST TARGET binding site such as BINDING SITE I, BINDING SITE II or BINDING SITE III. Table 2 illustrates the complementarity of the nucleotide sequences of PROS1 BINDING SITE, designated SEQ ID:3407, to the nucleotide sequence of VGAM274 RNA, herein designated VGAM RNA, also designated SEQ ID:609.

Another function of VGAM274 is therefore inhibition of Protein S (alpha) (PROS1, Accession XM_{—}113400). Accordingly, utilities of VGAM274 include diagnosis, prevention and treatment of diseases and clinical conditions associated with PROS1. FLJ20147 (Accession NM_{—}017687) is another VGAM274 host target gene. FLJ20147 BINDING SITE is HOST TARGET binding site found in the 3′ untranslated region of mRNA encoded by FLJ20147, corresponding to a HOST TARGET binding site such as BINDING SITE I, BINDING SITE II or BINDING SITE III. Table 2 illustrates the complementarity of the nucleotide sequences of FLJ20147 BINDING SITE, designated SEQ ID:1738, to the nucleotide sequence of VGAM274 RNA, herein designated VGAM RNA, also designated SEQ ID:609.

Another function of VGAM274 is therefore inhibition of FLJ20147 (Accession NM_{—}017687). Accordingly, utilities of VGAM274 include diagnosis, prevention and treatment of diseases and clinical conditions associated with FLJ20147. KIAA0737 (Accession NM_{—}014828) is another VGAM274 host target gene. KIAA0737 BINDING SITE is HOST TARGET binding site found in the 3′ untranslated region of mRNA encoded by KIAA0737, corresponding to a HOST TARGET binding site such as BINDING SITE I, BINDING SITE II or BINDING SITE III. Table 2 illustrates the complementarity of the nucleotide sequences of KIAA0737 BINDING SITE, designated SEQ ID:1562, to the nucleotide sequence of VGAM274 RNA, herein designated VGAM RNA, also designated SEQ ID:609.

Another function of VGAM274 is therefore inhibition of KIAA0737 (Accession NM_{—}014828). Accordingly, utilities of VGAM274 include diagnosis, prevention and treatment of diseases and clinical conditions associated with KIAA0737. FIG. 1 further provides a conceptual description of a novel bioinformatically detected viral gene of the present invention, referred to here as Viral Genomic Address Messenger 275 (VGAM275) viral gene, which modulates expression of respective host target genes thereof, the function and utility of which host target genes is known in the art.

VGAM275 is a novel bioinformatically detected regulatory, non protein coding, viral micro RNA (miRNA) gene. The method by which VGAM275 was detected is described hereinabove with reference to FIGS. 1-8.

VGAM275 gene, herein designated VGAM GENE, is a viral gene contained in the genome of Vaccinia Virus. VGAM275 host target gene, herein designated VGAM HOST TARGET GENE, is a human gene contained in the human genome.

VGAM275 gene encodes a VGAM275 precursor RNA, herein designated VGAM PRECURSOR RNA. Similar to other miRNA genes, and unlike most ordinary genes, VGAM275 precursor RNA does not encode a protein. A nucleotide sequence identical or highly similar to the nucleotide sequence of VGAM275 precursor RNA is designated SEQ ID:261, and is provided hereinbelow with reference to the sequence listing part. Nucleotide sequence SEQ ID:261 is located at position 157919 relative to the genome of Vaccinia Virus.

VGAM275 precursor RNA folds onto itself, forming VGAM275 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM275 folded precursor RNA into VGAM275 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM275 RNA is designated SEQ ID:610, and is provided hereinbelow with reference to the sequence listing part.

VGAM275 host target gene, herein designated VGAM HOST TARGET GENE, encodes a corresponding messenger RNA, VGAM275 host target RNA, herein designated VGAM HOST TARGET RNA. VGAM275 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM275 RNA, herein designated VGAM RNA, binds complementarily to one or more host target binding sites located in untranslated regions of VGAM275 host target RNA, herein designated VGAM HOST TARGET RNA. This complementary binding is due to the fact that the nucleotide sequence of VGAM275 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM275 RNA, herein designated VGAM RNA, may have a different number of host target binding sites in untranslated regions of a VGAM275 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM275 RNA, herein designated VGAM RNA, to host target binding sites on VGAM275 host target RNA, herein designated VGAM HOST TARGET RNA, such as BINDING SITE I, BINDING SITE II and BINDING SITE III, inhibits translation of VGAM275 host target RNA into VGAM275 host target protein, herein designated VGAM HOST TARGET PROTEIN. VGAM host target protein is therefore outlined by a broken line.

It is appreciated that VGAM275 host target gene, herein designated VGAM HOST TARGET GENE, in fact represents a plurality of VGAM275 host target genes. The mRNA of each one of this plurality of VGAM275 host target genes comprises one or more host target binding sites, each having a nucleotide sequence which is at least partly complementary to VGAM275 RNA, herein designated VGAM RNA, and which when bound by VGAM275 RNA causes inhibition of translation of respective one or more VGAM275 host target proteins.

It is further appreciated by one skilled in the art that the mode of translational inhibition illustrated by FIG. 1 with specific reference to translational inhibition exerted by VGAM275 gene, herein designated VGAM GENE, on one or more VGAM275 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM275 is inhibition of expression of host target genes, as part of a novel viral mechanism of attacking a host. Accordingly, utilities of VGAM275 include diagnosis, prevention and treatment of viral infection by Vaccinia Virus. Specific functions, and accordingly utilities, of VGAM275 correlate with, and may be deduced from, the identity of the host target genes which VGAM275 binds and inhibits, and the function of these host target genes, as elaborated hereinbelow.

Nucleotide sequences of the VGAM275 precursor RNA, herein designated VGAM PRECURSOR RNA, and of the ‘diced’ VGAM275 RNA, herein designated VGAM RNA, and a schematic representation of the secondary folding of VGAM275 folded precursor RNA, herein designated VGAM FOLDED PRECURSOR RNA, of VGAM275 are further described hereinbelow with reference to Table 1.

Nucleotide sequences of host target binding sites, such as BINDING SITE-I, BINDING SITE-II and BINDING SITE-III of FIG. 1, found on VGAM275 host target RNA, and schematic representation of the complementarity of each of these host target binding sites to VGAM275 RNA, herein designated VGAM RNA, are described hereinbelow with reference to Table 2.

As mentioned hereinabove with reference to FIG. 1, a function of VGAM275 gene, herein designated VGAM is inhibition of expression of VGAM275 target genes. It is appreciated that specific functions, and accordingly utilities, of VGAM275 correlate with, and may be deduced from, the identity of the target genes which VGAM275 binds and inhibits, and the function of these target genes, as elaborated hereinbelow.

Carbonic Anhydrase IV (CA4, Accession NM_{—}000717) is a VGAM275 host target gene. CA4 BINDING SITE is HOST TARGET binding site found in the 3′ untranslated region of mRNA encoded by CA4, corresponding to a HOST TARGET binding site such as BINDING SITE I, BINDING SITE II or BINDING SITE III. Table 2 illustrates the complementarity of the nucleotide sequences of CA4 BINDING SITE, designated SEQ ID:769, to the nucleotide sequence of VGAM275 RNA, herein designated VGAM RNA, also designated SEQ ID:610.

A function of VGAM275 is therefore inhibition of Carbonic Anhydrase IV (CA4, Accession NM_{—}000717), a gene which has a function of reversible hydratation of carbon dioxide. Accordingly, utilities of VGAM275 include diagnosis, prevention and treatment of diseases and clinical conditions associated with CA4. The function of CA4 has been established by previous studies. Carbonic anhydrases (CAs) are a family of zinc metalloenzymes. CA IV is a glycosylphosphatidylinositol-anchored membrane isozyme expressed on the luminal surfaces of pulmonary (and certain other) capillaries and on the luminal surface of proximal renal tubules. CA IV has ancient evolutionary status among CA isozymes. It is functionally important in CO2 and bicarbonate transport and has a possible role in inherited renal abnormalities of bicarbonate transport. Okuyama et al. (1992) isolated a full-length cDNA for human CA IV that contained a 47-bp 5-prime untranslated region, a 936-bp open reading frame, and a 122-bp 3-prime untranslated region. Okuyama et al. (1993) isolated a full-length genomic clone. They found that the 9.5-kb gene contains 8 exons and 7 introns. The first exon (exon 1a) encodes the signal sequence. Exon 7 encodes the C-terminus of the enzyme precursor, the C-terminus of the mature protein and the 120-bp sequence corresponding to the 3-prime untranslated region of the cDNA. Patients with renal abnormalities that selectively disturb bicarbonate transport, such as those with pure proximal renal tubular acidosis, are candidates for deficiency of carbonic anhydrase IV.

Full details of the abovementioned studies are described in the following publications, the disclosure of which are hereby incorporated by reference:
Okuyama, T.; Batanian, J. R.; Sly, W. S.: Genomic organization and localization of gene for human carbonic anhydrase IV to chromosome 17q. Genomics 16: 678-684, 1993.; and
Okuyama, T.; Sato, S.; Zhu, X. L.; Waheed, A.; Sly, W. S.: Human carbonic anhydrase IV: cDNA cloning, sequence comparison, and expression in COS cell membranes. Proc. Nat. Acad. Sci. 89.

Further studies establishing the function and utilities of CA4 are found in John Hopkins OMIM database record ID 114760, and in sited publications numbered 871-87 and 917-918 listed in the bibliography section hereinbelow, which are also hereby incorporated by reference. Lutheran Blood Group (Auberger b antigen included) (LU, Accession NM_{—}005581) is another VGAM275 host target gene. LU BINDING SITE is HOST TARGET binding site found in the 3′ untranslated region of mRNA encoded by LU, corresponding to a HOST TARGET binding site such as BINDING SITE I, BINDING SITE II or BINDING SITE III. Table 2 illustrates the complementarity of the nucleotide sequences of LU BINDING SITE, designated SEQ ID: 1227, to the nucleotide sequence of VGAM275 RNA, herein designated VGAM RNA, also designated SEQ ID:610.

Another function of VGAM275 is therefore inhibition of Lutheran Blood Group (Auberger b antigen included) (LU, Accession NM_{—}005581), a gene which is probably a receptor and may mediate intracellular signaling. Accordingly, utilities of VGAM275 include diagnosis, prevention and treatment of diseases and clinical conditions associated with LU. The function of LU has been established by previous studies. Lutheran and Secretor (Se; OMIM Ref. No. FUT2, 182100) are linked (review by Cook, 1965). Indeed this was the first autosomal linkage demonstrated in man, by Dr. Jan Mohr (1951) in Copenhagen, using Penrose's sib-pair method. See 111150 for description of a dominant Lutheran inhibitor comparable to Bombay (OMIM Ref. No. 211100) and the ABO blood groups. Myotonic dystrophy (OMIM Ref. No. 160900) is linked to Lutheran and Secretor, and Lewis (OMIM Ref. No. 111100) and Bombay are in the same linkage group with C3 (OMIM Ref. No. 120700) on chromosome 19. Gedde-Dahl et al. (1984) found linkage of Se and APOE (OMIM Ref. No. 107741)—peak Iod score 3.3 at recombination fraction 0.08 in males and 1.36 at 0.22 in females, and linkage of APOE and Lu with Iod score 4.52 at zero recombination in sexes combined. C3-APOE linkage gave Iod score 4.0 at theta 0.18 in males but 0.04 at theta 0.45 in females. Triple heterozygote families confirmed that APOE is on the Se side and on the Lu side of C3. A summarizing map was given (their FIG. 3). Lewis et al. (1988) demonstrated that APOC2 (OMIM Ref. No. 207750), Lu, and Se constitute a tightly linked gene cluster and argued that Lu and Se are on the long arm of chromosome 19 Parsons et al. (1997) determined the organization of the LU gene. The region encoding the open reading frame was arranged is 15 exons extending over approximately 11 kb on 19q13.2. They demonstrated that the Lu(a) mutation is a base change in exon 3 (252G-A; 111200.0001) encoding a change of arginine-77 in Lu(b) to histidine in Lu(a). The Lutheran glycoprotein has 5 predicted immunoglobulin super family domains; the base change responsible for the Lu(a)/Lu(b) polymorphism is located on domain 1. Parsons et al. (1997) also characterized the Au(a)/Au(b) polymorphism: a 1637A-G substitution in exon 12 (111200.0002) encoding a thr539-to-ala change on the G strand of domain 5. The predominant isoform of the Lutheran glycoprotein in most normal tissues is an 85,000-Da glycoprotein corresponding to the full-length LU cDNA, although in RBC membranes the minor 78,000-Da spliceoform can also be identified. The smaller form encoded by BCAM cDNA differs from Lutheran glycoprotein by lacking most of the cytoplasmic domain and arises by alternative splicing of an immature Lu gene transcript (Rahuel et al., 1996). Lutheran blood group glycoproteins are receptors for the extracellular matrix protein laminin. Studies suggest that these glycoproteins may contribute to vasoocclusion in sickle cell disease (OMIM Ref. No. 603903), and sickle cells can be shown to adhere to laminin isoforms containing the alpha-5 chain (laminin-10/11). Laminin alpha-5 (LAMA5; 601033) is present in the subendothelium and is also a constituent of bone marrow sinusoids, suggesting a role for the Lu/laminin interaction in erythropoiesis. Parsons et al. (2001) cloned and characterized mouse Lu. The human and mouse proteins share 72% sequence identity and both bind laminin-10/11 with high affinity.

Full details of the abovementioned studies are described in the following publications, the disclosure of which are hereby incorporated by reference:
Parsons, S. F.; Lee, G.; Spring, F. A.; Willig, T.-N.; Peters, L. L.; Gimm, J. A.; Tanner, M. J. A.; Mohandas, N.; Anstee, D. J.; Chasis, J. A.: Lutheran blood group glycoprotein and its newly characterized mouse homologue specifically bind alpha-5 chain-containing human laminin with high affinity. Blood 97: 312-320, 2001.; and
Cook, P. J. L.: The Lutheran-secretor recombination fraction in man: a possible sex difference. Ann. Hum. Genet. 28: 393-401, 1965.

Further studies establishing the function and utilities of LU are found in John Hopkins OMIM database record ID 111200, and in sited publications numbered 50-51, 49, 88-89, 2450-97, 88 and 99 listed in the bibliography section hereinbelow, which are also hereby incorporated by reference. Formin Binding Protein 3 (FNBP3, Accession XM_{—}087118) is another VGAM275 host target gene. FNBP3 BINDING SITE is HOST TARGET binding site found in the 5′ untranslated region of mRNA encoded by FNBP3, corresponding to a HOST TARGET binding site such as BINDING SITE I, BINDING SITE II or BINDING SITE III. Table 2 illustrates the complementarity of the nucleotide sequences of FNBP3 BINDING SITE, designated SEQ ID:3155, to the nucleotide sequence of VGAM275 RNA, herein designated VGAM RNA, also designated SEQ ID:610.

Another function of VGAM275 is therefore inhibition of Formin Binding Protein 3 (FNBP3, Accession XM_{—}087118). Accordingly, utilities of VGAM275 include diagnosis, prevention and treatment of diseases and clinical conditions associated with FNBP3. Sin3-associated Polypeptide, 18 kDa (SAP18, Accession NM_{—}005870) is another VGAM275 host target gene. SAP18 BINDING SITE is HOST TARGET binding site found in the 3′ untranslated region of mRNA encoded by SAP18, corresponding to a HOST TARGET binding site such as BINDING SITE I, BINDING SITE II or BINDING SITE III. Table 2 illustrates the complementarity of the nucleotide sequences of SAP18 BINDING SITE, designated SEQ ID:1258, to the nucleotide sequence of VGAM275 RNA, herein designated VGAM RNA, also designated SEQ ID:610.

Another function of VGAM275 is therefore inhibition of Sin3-associated Polypeptide, 18 kDa (SAP18, Accession NM_{—}005870). Accordingly, utilities of VGAM275 include diagnosis, prevention and treatment of diseases and clinical conditions associated with SAP18. SCYA4 (Accession NM_{—}002984) is another VGAM275 host target gene. SCYA4 BINDING SITE is HOST TARGET binding site found in the 3′ untranslated region of mRNA encoded by SCYA4, corresponding to a HOST TARGET binding site such as BINDING SITE I, BINDING SITE II or BINDING SITE III. Table 2 illustrates the complementarity of the nucleotide sequences of SCYA4 BINDING SITE, designated SEQ ID:978, to the nucleotide sequence of VGAM275 RNA, herein designated VGAM RNA, also designated SEQ ID:610.

Another function of VGAM275 is therefore inhibition of SCYA4 (Accession NM_{—}002984). Accordingly, utilities of VGAM275 include diagnosis, prevention and treatment of diseases and clinical conditions associated with SCYA4. Solute Carrier Family 6 (neurotransmitter transporter), Member 14 (SLC6A14, Accession NM_{—}007231) is another VGAM275 host target gene. SLC6A14 BINDING SITE is HOST TARGET binding site found in the 3′ untranslated region of mRNA encoded by SLC6A14, corresponding to a HOST TARGET binding site such as BINDING SITE I, BINDING SITE II or BINDING SITE III. Table 2 illustrates the complementarity of the nucleotide sequences of SLC6A14 BINDING SITE, designated SEQ ID:1368, to the nucleotide sequence of VGAM275 RNA, herein designated VGAM RNA, also designated SEQ ID:610.

Another function of VGAM275 is therefore inhibition of Solute Carrier Family 6 (neurotransmitter transporter), Member 14 (SLC6A14, Accession NM_{—}007231). Accordingly, utilities of VGAM275 include diagnosis, prevention and treatment of diseases and clinical conditions associated with SLC6A14. LOC158629 (Accession XM_{—}098972) is another VGAM275 host target gene. LOC158629 BINDING SITE is HOST TARGET binding site found in the 5′ untranslated region of mRNA encoded by LOC158629, corresponding to a HOST TARGET binding site such as BINDING SITE I, BINDING SITE II or BINDING SITE III. Table 2 illustrates the complementarity of the nucleotide sequences of LOC158629 BINDING SITE, designated SEQ ID:3393, to the nucleotide sequence of VGAM275 RNA, herein designated VGAM RNA, also designated SEQ ID:610.

Another function of VGAM275 is therefore inhibition of LOC158629 (Accession XM_{—}098972). Accordingly, utilities of VGAM275 include diagnosis, prevention and treatment of diseases and clinical conditions associated with LOC158629. LOC163115 (Accession XM_{—}092010) is another VGAM275 host target gene. LOC163115 BINDING SITE is HOST TARGET binding site found in the 5′ untranslated region of mRNA encoded by LOC163115, corresponding to a HOST TARGET binding site such as BINDING SITE I, BINDING SITE II or BINDING SITE III. Table 2 illustrates the complementarity of the nucleotide sequences of LOC163115 BINDING SITE, designated SEQ ID:3243, to the nucleotide sequence of VGAM275 RNA, herein designated VGAM RNA, also designated SEQ ID:610.

Another function of VGAM275 is therefore inhibition of LOC163115 (Accession XM_{—}092010). Accordingly, utilities of VGAM275 include diagnosis, prevention and treatment of diseases and clinical conditions associated with LOC163115. LOC200339 (Accession XM_{—}117226) is another VGAM275 host target gene. LOC200339 BINDING SITE is HOST TARGET binding site found in the 3′ untranslated region of mRNA encoded by LOC200339, corresponding to a HOST TARGET binding site such as BINDING SITE I, BINDING SITE II or BINDING SITE III. Table 2 illustrates the complementarity of the nucleotide sequences of LOC200339 BINDING SITE, designated SEQ ID:3476, to the nucleotide sequence of VGAM275 RNA, herein designated VGAM RNA, also designated SEQ ID:610.

Another function of VGAM275 is therefore inhibition of LOC200339 (Accession XM_{—}117226). Accordingly, utilities of VGAM275 include diagnosis, prevention and treatment of diseases and clinical conditions associated with LOC200339. LOC200953 (Accession XM_{—}117302) is another VGAM275 host target gene. LOC200953 BINDING SITE is HOST TARGET binding site found in the 5′ untranslated region of mRNA encoded by LOC200953, corresponding to a HOST TARGET binding site such as BINDING SITE I, BINDING SITE II or BINDING SITE III. Table 2 illustrates the complementarity of the nucleotide sequences of LOC200953 BINDING SITE, designated SEQ ID:3481, to the nucleotide sequence of VGAM275 RNA, herein designated VGAM RNA, also designated SEQ ID:610.

Another function of VGAM275 is therefore inhibition of LOC200953 (Accession XM_{—}117302). Accordingly, utilities of VGAM275 include diagnosis, prevention and treatment of diseases and clinical conditions associated with LOC200953. LOC255527 (Accession XM_{—}173026) is another VGAM275 host target gene. LOC255527 BINDING SITE is HOST TARGET binding site found in the 5′ untranslated region of mRNA encoded by LOC255527, corresponding to a HOST TARGET binding site such as BINDING SITE I, BINDING SITE II or BINDING SITE III. Table 2 illustrates the complementarity of the nucleotide sequences of LOC255527 BINDING SITE, designated SEQ ID:3718, to the nucleotide sequence of VGAM275 RNA, herein designated VGAM RNA, also designated SEQ ID:610.

Another function of VGAM275 is therefore inhibition of LOC255527 (Accession XM_{—}173026). Accordingly, utilities of VGAM275 include diagnosis, prevention and treatment of diseases and clinical conditions associated with LOC255527. LOC51334 (Accession NM_{—}016644) is another VGAM275 host target gene. LOC51334 BINDING SITE is HOST TARGET binding site found in the 3′ untranslated region of mRNA encoded by LOC51334, corresponding to a HOST TARGET binding site such as BINDING SITE I, BINDING SITE II or BINDING SITE III. Table 2 illustrates the complementarity of the nucleotide sequences of LOC51334 BINDING SITE, designated SEQ ID:1706, to the nucleotide sequence of VGAM275 RNA, herein designated VGAM RNA, also designated SEQ ID:610.

Another function of VGAM275 is therefore inhibition of LOC51334 (Accession NM_{—}016644). Accordingly, utilities of VGAM275 include diagnosis, prevention and treatment of diseases and clinical conditions associated with LOC51334. LOC92710 (Accession XM_{—}046811) is another VGAM275 host target gene. LOC92710 BINDING SITE is HOST TARGET binding site found in the 3′ untranslated region of mRNA encoded by LOC92710, corresponding to a HOST TARGET binding site such as BINDING SITE I, BINDING SITE II or BINDING SITE III. Table 2 illustrates the complementarity of the nucleotide sequences of LOC92710 BINDING SITE, designated SEQ ID:2884, to the nucleotide sequence of VGAM275 RNA, herein designated VGAM RNA, also designated SEQ ID:610.

Another function of VGAM275 is therefore inhibition of LOC92710 (Accession XM_{—}046811). Accordingly, utilities of VGAM275 include diagnosis, prevention and treatment of diseases and clinical conditions associated with LOC92710. FIG. 1 further provides a conceptual description of a novel bioinformatically detected viral gene of the present invention, referred to here as Viral Genomic Address Messenger 276 (VGAM276) viral gene, which modulates expression of respective host target genes thereof, the function and utility of which host target genes is known in the art.

VGAM276 is a novel bioinformatically detected regulatory, non protein coding, viral micro RNA (miRNA) gene. The method by which VGAM276 was detected is described hereinabove with reference to FIGS. 1-8.

VGAM276 gene, herein designated VGAM GENE, is a viral gene contained in the genome of Vaccinia Virus. VGAM276 host target gene, herein designated VGAM HOST TARGET GENE, is a human gene contained in the human genome.

VGAM276 gene encodes a VGAM276 precursor RNA, herein designated VGAM PRECURSOR RNA. Similar to other miRNA genes, and unlike most ordinary genes, VGAM276 precursor RNA does not encode a protein. A nucleotide sequence identical or highly similar to the nucleotide sequence of VGAM276 precursor RNA is designated SEQ ID:262, and is provided hereinbelow with reference to the sequence listing part. Nucleotide sequence SEQ ID:262 is located at position 156818 relative to the genome of Vaccinia Virus.

VGAM276 precursor RNA folds onto itself, forming VGAM276 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM276 folded precursor RNA into VGAM276 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM276 RNA is designated SEQ ID:611, and is provided hereinbelow with reference to the sequence listing part.

VGAM276 host target gene, herein designated VGAM HOST TARGET GENE, encodes a corresponding messenger RNA, VGAM276 host target RNA, herein designated VGAM HOST TARGET RNA. VGAM276 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM276 RNA, herein designated VGAM RNA, binds complementarily to one or more host target binding sites located in untranslated regions of VGAM276 host target RNA, herein designated VGAM HOST TARGET RNA. This complementary binding is due to the fact that the nucleotide sequence of VGAM276 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM276 RNA, herein designated VGAM RNA, may have a different number of host target binding sites in untranslated regions of a VGAM276 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM276 RNA, herein designated VGAM RNA, to host target binding sites on VGAM276 host target RNA, herein designated VGAM HOST TARGET RNA, such as BINDING SITE I, BINDING SITE II and BINDING SITE III, inhibits translation of VGAM276 host target RNA into VGAM276 host target protein, herein designated VGAM HOST TARGET PROTEIN. VGAM host target protein is therefore outlined by a broken line.

It is appreciated that VGAM276 host target gene, herein designated VGAM HOST TARGET GENE, in fact represents a plurality of VGAM276 host target genes. The mRNA of each one of this plurality of VGAM276 host target genes comprises one or more host target binding sites, each having a nucleotide sequence which is at least partly complementary to VGAM276 RNA, herein designated VGAM RNA, and which when bound by VGAM276 RNA causes inhibition of translation of respective one or more VGAM276 host target proteins.

It is further appreciated by one skilled in the art that the mode of translational inhibition illustrated by FIG. 1 with specific reference to translational inhibition exerted by VGAM276 gene, herein designated VGAM GENE, on one or more VGAM276 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM276 is inhibition of expression of host target genes, as part of a novel viral mechanism of attacking a host. Accordingly, utilities of VGAM276 include diagnosis, prevention and treatment of viral infection by Vaccinia Virus. Specific functions, and accordingly utilities, of VGAM276 correlate with, and may be deduced from, the identity of the host target genes which VGAM276 binds and inhibits, and the function of these host target genes, as elaborated hereinbelow.

Nucleotide sequences of the VGAM276 precursor RNA, herein designated VGAM PRECURSOR RNA, and of the ‘diced’ VGAM276 RNA, herein designated VGAM RNA, and a schematic representation of the secondary folding of VGAM276 folded precursor RNA, herein designated VGAM FOLDED PRECURSOR RNA, of VGAM276 are further described hereinbelow with reference to Table 1.

Nucleotide sequences of host target binding sites, such as BINDING SITE-I, BINDING SITE-II and BINDING SITE-III of FIG. 1, found on VGAM276 host target RNA, and schematic representation of the complementarity of each of these host target binding sites to VGAM276 RNA, herein designated VGAM RNA, are described hereinbelow with reference to Table 2.

As mentioned hereinabove with reference to FIG. 1, a function of VGAM276 gene, herein designated VGAM is inhibition of expression of VGAM276 target genes. It is appreciated that specific functions, and accordingly utilities, of VGAM276 correlate with, and may be deduced from, the identity of the target genes which VGAM276 binds and inhibits, and the function of these target genes, as elaborated hereinbelow.

Translin (TSN, Accession NM_{—}004622) is a VGAM276 host target gene. TSN BINDING SITE is HOST TARGET binding site found in the 3′ untranslated region of mRNA encoded by TSN, corresponding to a HOST TARGET binding site such as BINDING SITE I, BINDING SITE II or BINDING SITE III. Table 2 illustrates the complementarity of the nucleotide sequences of TSN BINDING SITE, designated SEQ ID:1136, to the nucleotide sequence of VGAM276 RNA, herein designated VGAM RNA, also designated SEQ ID:611.

A function of VGAM276 is therefore inhibition of Translin (TSN, Accession NM_{—}004622), a gene which is a DNA binding protein and involved in DNA repair, replication, or recombination. Accordingly, utilities of VGAM276 include diagnosis, prevention and treatment of diseases and clinical conditions associated with TSN. The function of TSN has been established by previous studies. Kasai et al. (1994) identified a protein they termed recombination hotspot-associated factor (RcHF1), which specifically binds to the signal-like sequences at the breakpoint junction of 8q24 and 1p32 in acute lymphoblastic leukemia (ALL) patients carrying t(8;14)(q24;q11) and t(1;14)(p32;q11) translocations involving the TCR delta-chain locus (TCRD; 186810). Aoki et al. (1994) showed that an analogous protein, which they designated BCLF1, specifically binds to a target sequence within the clustered breakpoint region of the BCL2 oncogene (OMIM Ref. No. 151430) in follicular lymphoma patients carrying t(14; 18)(q32;q21) translocations. It was proposed that these binding activities at recombination hotspot regions may play a crucial role in chromosomal translocations in lymphoid neoplasms. Aoki et al. (1995) purified the BCLF1 protein to homogeneity and determined that it is identical to RcHF1. Molecular gene cloning experiments revealed that the purified protein, which they named translin (TSN), is a previously undescribed DNA-binding protein with no significant similarity to known proteins. (The designation ‘translin’ came from selected letters in ‘translocation.’) In addition, Aoki et al. (1995) found that nuclear localization of translin was limited to lymphoid cell lines with rearranged Ig and processes such as DNA repair, replication, or recombination. In their native form, translin polypeptides form a multimeric structure that is responsible for its DNA binding activity. Aoki et al. (1997) found that the human and mouse translin genes have identical genomic structures consisting of 6 exons, 5 introns, and a GC-rich upstream region. By in situ hybridization and analysis of somatic cell hybrids, Aoki et al. (1997) mapped the human TSN gene to 2q21.1. Badge et al. (2000) studied a subtelomeric region at 16p13.3 that displays a 300-fold increase in crossovers compared to the genomic average rate. Segregation analysis of CEPH and other pedigrees yielded 6 paternal crossover breakpoints in the approximately 85-kb interval between the minisatellite loci D16S309 (MS205) and D16S83 (OMIM Ref. No. EKMDA2). Three crossovers were mapped to within the same small (less than 3 kb) interval, which did not colocalize with any tandem repeat array or expressed sequence. Sequence analysis revealed the presence of recombination-associated motifs and binding sites for translin. The authors concluded that this locus represents an intense male-specific recombination hotspot. Hosaka et al. (2000) demonstrated that the presence of the translin binding motif may be one of the important determinants for the location of breakpoints in the TLS (OMIM Ref. No. 137070) and CHOP (OMIM Ref. No. 126337) genes which are fused by translocation t(12; 16) in liposarcomas.

Full details of the abovementioned studies are described in the following publications, the disclosure of which are hereby incorporated by reference:
Badge, R. M.; Yardley, J.; Jeffreys, A. J.; Armour, J. A. L.: Crossover breakpoint mapping identifies a subtelomeric hotspot for male meiotic recombination. Hum. Molec. Genet. 9: 1239-1244, 2000.; and
Hosaka, T.; Kanoe, H.; Nakayama, T.; Murakami, H.; Yamamoto, H.; Nakamata, T.; Tsuboyama, T.; Oka, M.; Kasai, M.; Sasaki, M. S.; Nakamura, T.; Toguchida, J.: Translin binds to the sequ.

Further studies establishing the function and utilities of TSN are found in John Hopkins OMIM database record ID 600575, and in sited publications numbered 2154-215 and 2218-2221 listed in the bibliography section hereinbelow, which are also hereby incorporated by reference. FLJ20039 (Accession NM_{—}017635) is another VGAM276 host target gene. FLJ20039 BINDING SITE is HOST TARGET binding site found in the 3′ untranslated region of mRNA encoded by FLJ20039, corresponding to a HOST TARGET binding site such as BINDING SITE I, BINDING SITE II or BINDING SITE III. Table 2 illustrates the complementarity of the nucleotide sequences of FLJ20039 BINDING SITE, designated SEQ ID:1734, to the nucleotide sequence of VGAM276 RNA, herein designated VGAM RNA, also designated SEQ ID:611.

Another function of VGAM276 is therefore inhibition of FLJ20039 (Accession NM_{—}017635). Accordingly, utilities of VGAM276 include diagnosis, prevention and treatment of diseases and clinical conditions associated with FLJ20039. KIAA1204 (Accession XM_{—}045011) is another VGAM276 host target gene. KIAA1204 BINDING SITE is HOST TARGET binding site found in the 3′ untranslated region of mRNA encoded by KIAA1204, corresponding to a HOST TARGET binding site such as BINDING SITE I, BINDING SITE II or BINDING SITE III. Table 2 illustrates the complementarity of the nucleotide sequences of KIAA1204 BINDING SITE, designated SEQ ID:2849, to the nucleotide sequence of VGAM276 RNA, herein designated VGAM RNA, also designated SEQ ID:611.

Another function of VGAM276 is therefore inhibition of KIAA1204 (Accession XM_{—}045011). Accordingly, utilities of VGAM276 include diagnosis, prevention and treatment of diseases and clinical conditions associated with KIAA1204. Olfactory Receptor, Family 2, Sub family C, Member 3 (OR2C3, Accession XM_{—}060575) is another VGAM276 host target gene. OR2C3 BINDING SITE is HOST TARGET binding site found in the 3′ untranslated region of mRNA encoded by OR2C3, corresponding to a HOST TARGET binding site such as BINDING SITE I, BINDING SITE II or BINDING SITE III. Table 2 illustrates the complementarity of the nucleotide sequences of OR2C3 BINDING SITE, designated SEQ ID:3023, to the nucleotide sequence of VGAM276 RNA, herein designated VGAM RNA, also designated SEQ ID:611.

Another function of VGAM276 is therefore inhibition of Olfactory Receptor, Family 2, Sub family C, Member 3 (OR2C3, Accession XM_{—}060575). Accordingly, utilities of VGAM276 include diagnosis, prevention and treatment of diseases and clinical conditions associated with OR2C3. QKI (Accession XM_{—}037438) is another VGAM276 host target gene. QKI BINDING SITE is HOST TARGET binding site found in the 3′ untranslated region of mRNA encoded by QKI, corresponding to a HOST TARGET binding site such as BINDING SITE I, BINDING SITE II or BINDING SITE III. Table 2 illustrates the complementarity of the nucleotide sequences of QKI BINDING SITE, designated SEQ ID:2722, to the nucleotide sequence of VGAM276 RNA, herein designated VGAM RNA, also designated SEQ ID:611.

Another function of VGAM276 is therefore inhibition of QKI (Accession XM_{—}037438). Accordingly, utilities of VGAM276 include diagnosis, prevention and treatment of diseases and clinical conditions associated with QKI. TIP120A (Accession NM_{—}018448) is another VGAM276 host target gene. TIP120A BINDING SITE is HOST TARGET binding site found in the 3′ untranslated region of mRNA encoded by TIP120A, corresponding to a HOST TARGET binding site such as BINDING SITE I, BINDING SITE II or BINDING SITE III. Table 2 illustrates the complementarity of the nucleotide sequences of TIP120A BINDING SITE, designated SEQ ID:1828, to the nucleotide sequence of VGAM276 RNA, herein designated VGAM RNA, also designated SEQ ID:611.

Another function of VGAM276 is therefore inhibition of TIP120A (Accession NM_{—}018448). Accordingly, utilities of VGAM276 include diagnosis, prevention and treatment of diseases and clinical conditions associated with TIP120A. LOC90750 (Accession XM_{—}033868) is another VGAM276 host target gene. LOC90750 BINDING SITE is HOST TARGET binding site found in the 3′ untranslated region of mRNA encoded by LOC90750, corresponding to a HOST TARGET binding site such as BINDING SITE I, BINDING SITE II or BINDING SITE III. Table 2 illustrates the complementarity of the nucleotide sequences of LOC90750 BINDING SITE, designated SEQ ID:2673, to the nucleotide sequence of VGAM276 RNA, herein designated VGAM RNA, also designated SEQ ID:611.

Another function of VGAM276 is therefore inhibition of LOC90750 (Accession XM_{—}033868). Accordingly, utilities of VGAM276 include diagnosis, prevention and treatment of diseases and clinical conditions associated with LOC90750. LOC92912 (Accession XM_{—}047970) is another VGAM276 host target gene. LOC92912 BINDING SITE is HOST TARGET binding site found in the 3′ untranslated region of mRNA encoded by LOC92912, corresponding to a HOST TARGET binding site such as BINDING SITE I, BINDING SITE II or BINDING SITE III. Table 2 illustrates the complementarity of the nucleotide sequences of LOC92912 BINDING SITE, designated SEQ ID:2898, to the nucleotide sequence of VGAM276 RNA, herein designated VGAM RNA, also designated SEQ ID:611.

Another function of VGAM276 is therefore inhibition of LOC92912 (Accession XM_{—}047970). Accordingly, utilities of VGAM276 include diagnosis, prevention and treatment of diseases and clinical conditions associated with LOC92912. FIG. 1 further provides a conceptual description of a novel bioinformatically detected viral gene of the present invention, referred to here as Viral Genomic Address Messenger 277 (VGAM277) viral gene, which modulates expression of respective host target genes thereof, the function and utility of which host target genes is known in the art.

VGAM277 is a novel bioinformatically detected regulatory, non protein coding, viral micro RNA (miRNA) gene. The method by which VGAM277 was detected is described hereinabove with reference to FIGS. 1-8.

VGAM277 gene, herein designated VGAM GENE, is a viral gene contained in the genome of Vaccinia Virus. VGAM277 host target gene, herein designated VGAM HOST TARGET GENE, is a human gene contained in the human genome.

VGAM277 gene encodes a VGAM277 precursor RNA, herein designated VGAM PRECURSOR RNA. Similar to other miRNA genes, and unlike most ordinary genes, VGAM277 precursor RNA does not encode a protein. A nucleotide sequence identical or highly similar to the nucleotide sequence of VGAM277 precursor RNA is designated SEQ ID:263, and is provided hereinbelow with reference to the sequence listing part. Nucleotide sequence SEQ ID:263 is located at position 157748 relative to the genome of Vaccinia Virus.

VGAM277 precursor RNA folds onto itself, forming VGAM277 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM277 folded precursor RNA into VGAM277 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM277 RNA is designated SEQ ID:612, and is provided hereinbelow with reference to the sequence listing part.

VGAM277 host target gene, herein designated VGAM HOST TARGET GENE, encodes a corresponding messenger RNA, VGAM277 host target RNA, herein designated VGAM HOST TARGET RNA. VGAM277 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM277 RNA, herein designated VGAM RNA, binds complementarily to one or more host target binding sites located in untranslated regions of VGAM277 host target RNA, herein designated VGAM HOST TARGET RNA. This complementary binding is due to the fact that the nucleotide sequence of VGAM277 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM277 RNA, herein designated VGAM RNA, may have a different number of host target binding sites in untranslated regions of a VGAM277 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM277 RNA, herein designated VGAM RNA, to host target binding sites on VGAM277 host target RNA, herein designated VGAM HOST TARGET RNA, such as BINDING SITE I, BINDING SITE II and BINDING SITE III, inhibits translation of VGAM277 host target RNA into VGAM277 host target protein, herein designated VGAM HOST TARGET PROTEIN. VGAM host target protein is therefore outlined by a broken line.

It is appreciated that VGAM277 host target gene, herein designated VGAM HOST TARGET GENE, in fact represents a plurality of VGAM277 host target genes. The mRNA of each one of this plurality of VGAM277 host target genes comprises one or more host target binding sites, each having a nucleotide sequence which is at least partly complementary to VGAM277 RNA, herein designated VGAM RNA, and which when bound by VGAM277 RNA causes inhibition of translation of respective one or more VGAM277 host target proteins.

It is further appreciated by one skilled in the art that the mode of translational inhibition illustrated by FIG. 1 with specific reference to translational inhibition exerted by VGAM277 gene, herein designated VGAM GENE, on one or more VGAM277 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM277 is inhibition of expression of host target genes, as part of a novel viral mechanism of attacking a host. Accordingly, utilities of VGAM277 include diagnosis, prevention and treatment of viral infection by Vaccinia Virus. Specific functions, and accordingly utilities, of VGAM277 correlate with, and may be deduced from, the identity of the host target genes which VGAM277 binds and inhibits, and the function of these host target genes, as elaborated hereinbelow.

Nucleotide sequences of the VGAM277 precursor RNA, herein designated VGAM PRECURSOR RNA, and of the ‘diced’ VGAM277 RNA, herein designated VGAM RNA, and a schematic representation of the secondary folding of VGAM277 folded precursor RNA, herein designated VGAM FOLDED PRECURSOR RNA, of VGAM277 are further described hereinbelow with reference to Table 1.

Nucleotide sequences of host target binding sites, such as BINDING SITE-I, BINDING SITE-II and BINDING SITE-III of FIG. 1, found on VGAM277 host target RNA, and schematic representation of the complementarity of each of these host target binding sites to VGAM277 RNA, herein designated VGAM RNA, are described hereinbelow with reference to Table 2.

As mentioned hereinabove with reference to FIG. 1, a function of VGAM277 gene, herein designated VGAM is inhibition of expression of VGAM277 target genes. It is appreciated that specific functions, and accordingly utilities, of VGAM277 correlate with, and may be deduced from, the identity of the target genes which VGAM277 binds and inhibits, and the function of these target genes, as elaborated hereinbelow.

BTB and CNC Homology 1, Basic Leucine Zipper Transcription Factor 2 (BACH2, Accession NM_{—}021813) is a VGAM277 host target gene. BACH2 BINDING SITE is HOST TARGET binding site found in the 3′ untranslated region of mRNA encoded by BACH2, corresponding to a HOST TARGET binding site such as BINDING SITE I, BINDING SITE II or BINDING SITE III. Table 2 illustrates the complementarity of the nucleotide sequences of BACH2 BINDING SITE, designated SEQ ID:1962, to the nucleotide sequence of VGAM277 RNA, herein designated VGAM RNA, also designated SEQ ID:612.

A function of VGAM277 is therefore inhibition of BTB and CNC Homology 1, Basic Leucine Zipper Transcription Factor 2 (BACH2, Accession NM_{—}021813), a gene which acts as repressor or activator, binds to maf recognition elements. Accordingly, utilities of VGAM277 include diagnosis, prevention and treatment of diseases and clinical conditions associated with BACH2. The function of BACH2 and its association with various diseases and clinical conditions, has been established by previous studies, as described hereinabove with reference to VGAM119. Dual Specificity Phosphatase 11 (RNA/RNP complex 1-interacting) (DUSP11, Accession NM_{—}003584) is another VGAM277 host target gene. DUSP11 BINDING SITE is HOST TARGET binding site found in the 3′ untranslated region of mRNA encoded by DUSP11, corresponding to a HOST TARGET binding site such as BINDING SITE I, BINDING SITE II or BINDING SITE III. Table 2 illustrates the complementarity of the nucleotide sequences of DUSP11 BINDING SITE, designated SEQ ID: 1039, to the nucleotide sequence of VGAM277 RNA, herein designated VGAM RNA, also designated SEQ ID:612.

Another function of VGAM277 is therefore inhibition of Dual Specificity Phosphatase 11 (RNA/RNP complex 1-interacting) (DUSP11, Accession NM_{—}003584). Accordingly, utilities of VGAM277 include diagnosis, prevention and treatment of diseases and clinical conditions associated with DUSP11. Epidermal Growth Factor Receptor Pathway Substrate 8 (EPS8, Accession NM_{—}004447) is another VGAM277 host target gene. EPS8 BINDING SITE is HOST TARGET binding site found in the 3′ untranslated region of mRNA encoded by EPS8, corresponding to a HOST TARGET binding site such as BINDING SITE I, BINDING SITE II or BINDING SITE III. Table 2 illustrates the complementarity of the nucleotide sequences of EPS8 BINDING SITE, designated SEQ ID:1114, to the nucleotide sequence of VGAM277 RNA, herein designated VGAM RNA, also designated SEQ ID:612.

Another function of VGAM277 is therefore inhibition of Epidermal Growth Factor Receptor Pathway Substrate 8 (EPS8, Accession NM_{—}004447), a gene which has a role in normal and neoplastic cell proliferation; contains an SH3 motif. Accordingly, utilities of VGAM277 include diagnosis, prevention and treatment of diseases and clinical conditions associated with EPS8. The function of EPS8 has been established by previous studies. Using an expression cloning approach for the study of epidermal growth factor (EGF) receptor (EGFR; 131550)-activated signaling, Wong et al. (1994) found a number of murine cDNA clones referred to as eps, for egfr-pathway-substrate. (See 600051 for one of these, EPS15.) One of the clones encoded a protein of 97 kD, designated eps8, which was phosphorylated in vivo by several receptor tyrosine kinases (Fazioli et al., 1993). In addition to a previously identified SH3 domain, Wong et al. (1994) found that the predicted amino acid sequence of human EPS8 showed a nonrandom distribution of prolines, clustered in a way to suggest SH3-binding sites and a putative PH domain. EPS8 was expressed in all epithelial and fibroblast cell lines examined and in some, but not all, hematopoietic cells. An essential function of EPS8 in cell growth regulation was underscored by its conservation during evolution where EPS8-related sequences were detected as early as in S. cerevisiae. EGFR signaling involves small GTPases of the Rho family, and EGFR trafficking involves small GTPases of the Rab family. Lanzetti et al. (2000) reported that the EPS8 protein connects these signaling pathways. EPS8 is a substrate of EGFR that is held in a complex with SOS1 by the adaptor protein E3B1, thereby mediating activation of RAC. Through its SH3 domain, EPS8 interacts with RNTRE (OMIM Ref. No. 605405). Lanzetti et al. (2000) showed that RNTRE is a RAB5 (OMIM Ref. No. 179512) GTPase-activating protein whose activity is regulated by EGFR. By entering in a complex with EPS8, RNTRE acts on RAB5 and inhibits internalization of the EGFR. Furthermore, RNTRE diverts EPS8 from its RAC-activating function, resulting in the attenuation of RAC signaling. Thus, depending on its state of association with E3B1 or RNTRE, EPS8 participates in both EGFR signaling through RAC and EGFR trafficking through RAB5. Wong et al. (1994) mapped the human EPS8 locus to 12q23-q24 by study of human-rodent somatic cell hybrid DNAs and by fluorescence in situ hybridization. In an study of candidate genes for Noonan syndrome (OMIM Ref. No. 163950), Ion et al. (2000) reassigned the map position of EPS8 to 12q13 using FISH.

Full details of the abovementioned studies are described in the following publications, the disclosure of which are hereby incorporated by reference:
Wong, W. T.; Carlomagno, F.; Druck, T.; Barletta, C.; Croce, C. M.; Huebner, K.; Kraus, M. H.; Di Fiore, P. P.: Evolutionary conservation of the EPS8 gene and its mapping to human chromosome 12q23-q24. Oncogene 9: 3057-3061, 1994.; and
Lanzetti, L.; Rybin, V.; Malabarba, M. G.; Christoforidis, S.; Scita, G.; Zerial, M.; Di Fiore, P. P.: The Eps8 protein coordinates EGF receptor signalling through Rac and trafficking t.

Further studies establishing the function and utilities of EPS8 are found in John Hopkins OMIM database record ID 600206, and in sited publications numbered 335, 44 and 495 listed in the bibliography section hereinbelow, which are also hereby incorporated by reference. Thymidine Kinase 1, Soluble (TK1, Accession NM_{—}003258) is another VGAM277 host target gene. TK1 BINDING SITE is HOST TARGET binding site found in the 3′ untranslated region of mRNA encoded by TK1, corresponding to a HOST TARGET binding site such as BINDING SITE I, BINDING SITE II or BINDING SITE III. Table 2 illustrates the complementarity of the nucleotide sequences of TK1 BINDING SITE, designated SEQ ID: 1005, to the nucleotide sequence of VGAM277 RNA, herein designated VGAM RNA, also designated SEQ ID:612.

Another function of VGAM277 is therefore inhibition of Thymidine Kinase 1, Soluble (TK1, Accession NM_{—}003258). Accordingly, utilities of VGAM277 include diagnosis, prevention and treatment of diseases and clinical conditions associated with TK1. Transient Receptor Potential Cation Channel, Sub family C, Member 6 (TRPC6, Accession NM_{—}004621) is another VGAM277 host target gene. TRPC6 BINDING SITE is HOST TARGET binding site found in the 3′ untranslated region of mRNA encoded by TRPC6, corresponding to a HOST TARGET binding site such as BINDING SITE I, BINDING SITE II or BINDING SITE III. Table 2 illustrates the complementarity of the nucleotide sequences of TRPC6 BINDING SITE, designated SEQ ID:1134, to the nucleotide sequence of VGAM277 RNA, herein designated VGAM RNA, also designated SEQ ID:612.

Another function of VGAM277 is therefore inhibition of Transient Receptor Potential Cation Channel, Sub family C, Member 6 (TRPC6, Accession NM_{—}004621), a gene which has calcium channel activity. Accordingly, utilities of VGAM277 include diagnosis, prevention and treatment of diseases and clinical conditions associated with TRPC6. The function of TRPC6 and its association with various diseases and clinical conditions, has been established by previous studies, as described hereinabove with reference to VGAM97. KIAA0924 (Accession NM_{—}014897) is another VGAM277 host target gene. KIAA0924 BINDING SITE is HOST TARGET binding site found in the 3′ untranslated region of mRNA encoded by KIAA0924, corresponding to a HOST TARGET binding site such as BINDING SITE I, BINDING SITE II or BINDING SITE III. Table 2 illustrates the complementarity of the nucleotide sequences of KIAA0924 BINDING SITE, designated SEQ ID:1577, to the nucleotide sequence of VGAM277 RNA, herein designated VGAM RNA, also designated SEQ ID:612.

Another function of VGAM277 is therefore inhibition of KIAA0924 (Accession NM_{—}014897). Accordingly, utilities of VGAM277 include diagnosis, prevention and treatment of diseases and clinical conditions associated with KIAA0924. KIAA1946 (Accession XM_{—}092459) is another VGAM277 host target gene. KIAA1946 BINDING SITE is HOST TARGET binding site found in the 3′ untranslated region of mRNA encoded by KIAA1946, corresponding to a HOST TARGET binding site such as BINDING SITE I, BINDING SITE II or BINDING SITE III. Table 2 illustrates the complementarity of the nucleotide sequences of KIAA1946 BINDING SITE, designated SEQ ID:3247, to the nucleotide sequence of VGAM277 RNA, herein designated VGAM RNA, also designated SEQ ID:612.

Another function of VGAM277 is therefore inhibition of KIAA1946 (Accession XM_{—}092459). Accordingly, utilities of VGAM277 include diagnosis, prevention and treatment of diseases and clinical conditions associated with KIAA1946. Oxysterol Binding Protein-like 3 (OSBPL3, Accession NM_{—}015550) is another VGAM277 host target gene. OSBPL3 BINDING SITE is HOST TARGET binding site found in the 3′ untranslated region of mRNA encoded by OSBPL3, corresponding to a HOST TARGET binding site such as BINDING SITE I, BINDING SITE II or BINDING SITE III. Table 2 illustrates the complementarity of the nucleotide sequences of OSBPL3 BINDING SITE, designated SEQ ID:1635, to the nucleotide sequence of VGAM277 RNA, herein designated VGAM RNA, also designated SEQ ID:612.

Another function of VGAM277 is therefore inhibition of Oxysterol Binding Protein-like 3 (OSBPL3, Accession NM_{—}015550). Accordingly, utilities of VGAM277 include diagnosis, prevention and treatment of diseases and clinical conditions associated with OSBPL3. Protein Serine Kinase H1 (PSKH1, Accession XM_{—}043047) is another VGAM277 host target gene. PSKH1 BINDING SITE is HOST TARGET binding site found in the 3′ untranslated region of mRNA encoded by PSKH1, corresponding to a HOST TARGET binding site such as BINDING SITE I, BINDING SITE II or BINDING SITE III. Table 2 illustrates the complementarity of the nucleotide sequences of PSKH1 BINDING SITE, designated SEQ ID:2810, to the nucleotide sequence of VGAM277 RNA, herein designated VGAM RNA, also designated SEQ ID:612.

Another function of VGAM277 is therefore inhibition of Protein Serine Kinase H1 (PSKH1, Accession XM_{—}043047). Accordingly, utilities of VGAM277 include diagnosis, prevention and treatment of diseases and clinical conditions associated with PSKH1. URB (Accession XM_{—}087331) is another VGAM277 host target gene. URB BINDING SITE is HOST TARGET binding site found in the 5′ untranslated region of mRNA encoded by URB, corresponding to a HOST TARGET binding site such as BINDING SITE I, BINDING SITE II or BINDING SITE III. Table 2 illustrates the complementarity of the nucleotide sequences of URB BINDING SITE, designated SEQ ID:3162, to the nucleotide sequence of VGAM277 RNA, herein designated VGAM RNA, also designated SEQ ID:612.

Another function of VGAM277 is therefore inhibition of URB (Accession XM_{—}087331). Accordingly, utilities of VGAM277 include diagnosis, prevention and treatment of diseases and clinical conditions associated with URB. FIG. 1 further provides a conceptual description of a novel bioinformatically detected viral gene of the present invention, referred to here as Viral Genomic Address Messenger 278 (VGAM278) viral gene, which modulates expression of respective host target genes thereof, the function and utility of which host target genes is known in the art.

VGAM278 is a novel bioinformatically detected regulatory, non protein coding, viral micro RNA (miRNA) gene. The method by which VGAM278 was detected is described hereinabove with reference to FIGS. 1-8.

VGAM278 gene, herein designated VGAM GENE, is a viral gene contained in the genome of Vaccinia Virus. VGAM278 host target gene, herein designated VGAM HOST TARGET GENE, is a human gene contained in the human genome.

VGAM278 gene encodes a VGAM278 precursor RNA, herein designated VGAM PRECURSOR RNA. Similar to other miRNA genes, and unlike most ordinary genes, VGAM278 precursor RNA does not encode a protein. A nucleotide sequence identical or highly similar to the nucleotide sequence of VGAM278 precursor RNA is designated SEQ ID:264, and is provided hereinbelow with reference to the sequence listing part. Nucleotide sequence SEQ ID:264 is located at position 158423 relative to the genome of Vaccinia Virus.

VGAM278 precursor RNA folds onto itself, forming VGAM278 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM278 folded precursor RNA into VGAM278 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 94%) nucleotide sequence of VGAM278 RNA is designated SEQ ID:613, and is provided hereinbelow with reference to the sequence listing part.

VGAM278 host target gene, herein designated VGAM HOST TARGET GENE, encodes a corresponding messenger RNA, VGAM278 host target RNA, herein designated VGAM HOST TARGET RNA. VGAM278 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM278 RNA, herein designated VGAM RNA, binds complementarily to one or more host target binding sites located in untranslated regions of VGAM278 host target RNA, herein designated VGAM HOST TARGET RNA. This complementary binding is due to the fact that the nucleotide sequence of VGAM278 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM278 RNA, herein designated VGAM RNA, may have a different number of host target binding sites in untranslated regions of a VGAM278 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM278 RNA, herein designated VGAM RNA, to host target binding sites on VGAM278 host target RNA, herein designated VGAM HOST TARGET RNA, such as BINDING SITE I, BINDING SITE II and BINDING SITE III, inhibits translation of VGAM278 host target RNA into VGAM278 host target protein, herein designated VGAM HOST TARGET PROTEIN. VGAM host target protein is therefore outlined by a broken line.

It is appreciated that VGAM278 host target gene, herein designated VGAM HOST TARGET GENE, in fact represents a plurality of VGAM278 host target genes. The mRNA of each one of this plurality of VGAM278 host target genes comprises one or more host target binding sites, each having a nucleotide sequence which is at least partly complementary to VGAM278 RNA, herein designated VGAM RNA, and which when bound by VGAM278 RNA causes inhibition of translation of respective one or more VGAM278 host target proteins.

It is further appreciated by one skilled in the art that the mode of translational inhibition illustrated by FIG. 1 with specific reference to translational inhibition exerted by VGAM278 gene, herein designated VGAM GENE, on one or more VGAM278 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM278 is inhibition of expression of host target genes, as part of a novel viral mechanism of attacking a host. Accordingly, utilities of VGAM278 include diagnosis, prevention and treatment of viral infection by Vaccinia Virus. Specific functions, and accordingly utilities, of VGAM278 correlate with, and may be deduced from, the identity of the host target genes which VGAM278 binds and inhibits, and the function of these host target genes, as elaborated hereinbelow.

Nucleotide sequences of the VGAM278 precursor RNA, herein designated VGAM PRECURSOR RNA, and of the ‘diced’ VGAM278 RNA, herein designated VGAM RNA, and a schematic representation of the secondary folding of VGAM278 folded precursor RNA, herein designated VGAM FOLDED PRECURSOR RNA, of VGAM278 are further described hereinbelow with reference to Table 1.

Nucleotide sequences of host target binding sites, such as BINDING SITE-I, BINDING SITE-II and BINDING SITE-III of FIG. 1, found on VGAM278 host target RNA, and schematic representation of the complementarity of each of these host target binding sites to VGAM278 RNA, herein designated VGAM RNA, are described hereinbelow with reference to Table 2.

As mentioned hereinabove with reference to FIG. 1, a function of VGAM278 gene, herein designated VGAM is inhibition of expression of VGAM278 target genes. It is appreciated that specific functions, and accordingly utilities, of VGAM278 correlate with, and may be deduced from, the identity of the target genes which VGAM278 binds and inhibits, and the function of these target genes, as elaborated hereinbelow.

Plastin 3 (T isoform) (PLS3, Accession NM_{—}005032) is a VGAM278 host target gene. PLS3 BINDING SITE is HOST TARGET binding site found in the 3′ untranslated region of mRNA encoded by PLS3, corresponding to a HOST TARGET binding site such as BINDING SITE I, BINDING SITE II or BINDING SITE III. Table 2 illustrates the complementarity of the nucleotide sequences of PLS3 BINDING SITE, designated SEQ ID:1174, to the nucleotide sequence of VGAM278 RNA, herein designated VGAM RNA, also designated SEQ ID:613.

A function of VGAM278 is therefore inhibition of Plastin 3 (T isoform) (PLS3, Accession NM_{—}005032), a gene which binds actin. Accordingly, utilities of VGAM278 include diagnosis, prevention and treatment of diseases and clinical conditions associated with PLS3. The function of PLS3 has been established by previous studies. Plastins are a family of actin-binding proteins that are differentially expressed in normal and malignant cells. Lin et al. (1988) isolated partial cDNAs encoding T-plastin and L-plastin (OMIM Ref. No. 153430) from a transformed human fibroblast cDNA library. The C-terminal 570 amino acids of the T-plastin and L-plastin proteins are 83% identical. By 2-dimensional gel electrophoresis of human cell extracts, Lin et al. (1988) showed that T-plastin is expressed as 2 equally abundant isoforms. Northern blot analysis revealed that T-plastin is expressed as a 3.4-kb mRNA in normal cells of solid tissues and in transformed fibroblasts. Using anchored PCR, Lin et al. (1990) identified the 5-prime end of the T-plastin mRNA. The T-plastin transcript has 2 possible translation initiation codons which would result in predicted 627- and 630-amino acid proteins. The authors constructed 2 modified T-plastin cDNAs containing either the first or the second initiation codon. Expression of these cDNAs in E. coli resulted in the synthesis of 2 distinct T-plastins with the same isoelectric points and apparent molecular weights as the 2 T-plastins present in human cells. Lin et al. (1990) found that T-plastin contains a potential calcium-binding site near the N terminus. Lin et al. (1993) reported that both the L-plastin and T-plastin genes contain 16 exons and span approximately 90 kb

Full details of the abovementioned studies are described in the following publications, the disclosure of which are hereby incorporated by reference:
Lin, C.-S.; Aebersold, R. H.; Leavitt, J.: Correction of the N-terminal sequences of the human plastin isoforms by using anchored polymerase chain reaction: identification of a potential calcium-binding domain. Molec. Cell. Biol. 10: 1818-1821, 1990.; and
Lin, C.-S.; Park, T.; Chen, Z. P.; Leavitt, J.: Human plastin genes: comparative gene structure, chromosome location, and differential expression in normal and neoplastic cells. J. Biol.

Further studies establishing the function and utilities of PLS3 are found in John Hopkins OMIM database record ID 300131, and in sited publications numbered 13 and 2525 listed in the bibliography section hereinbelow, which are also hereby incorporated by reference. LOC121227 (Accession XM_{—}058545) is another VGAM278 host target gene. LOC121227 BINDING SITE is HOST TARGET binding site found in the 3′ untranslated region of mRNA encoded by LOC121227, corresponding to a HOST TARGET binding site such as BINDING SITE I, BINDING SITE II or BINDING SITE III. Table 2 illustrates the complementarity of the nucleotide sequences of LOC121227 BINDING SITE, designated SEQ ID:2989, to the nucleotide sequence of VGAM278 RNA, herein designated VGAM RNA, also designated SEQ ID:613.

Another function of VGAM278 is therefore inhibition of LOC121227 (Accession XM_{—}058545). Accordingly, utilities of VGAM278 include diagnosis, prevention and treatment of diseases and clinical conditions associated with LOC121227. LOC145786 (Accession XM_{—}096860) is another VGAM278 host target gene. LOC145786 BINDING SITE is HOST TARGET binding site found in the 3′ untranslated region of mRNA encoded by LOC145786, corresponding to a HOST TARGET binding site such as BINDING SITE I, BINDING SITE II or BINDING SITE III. Table 2 illustrates the complementarity of the nucleotide sequences of LOC145786 BINDING SITE, designated SEQ ID:3277, to the nucleotide sequence of VGAM278 RNA, herein designated VGAM RNA, also designated SEQ ID:613.

Another function of VGAM278 is therefore inhibition of LOC145786 (Accession XM_{—}096860). Accordingly, utilities of VGAM278 include diagnosis, prevention and treatment of diseases and clinical conditions associated with LOC145786. LOC149650 (Accession XM_{—}086623) is another VGAM278 host target gene. LOC149650 BINDING SITE is HOST TARGET binding site found in the 3′ untranslated region of mRNA encoded by LOC149650, corresponding to a HOST TARGET binding site such as BINDING SITE I, BINDING SITE II or BINDING SITE III. Table 2 illustrates the complementarity of the nucleotide sequences of LOC149650 BINDING SITE, designated SEQ ID:3136, to the nucleotide sequence of VGAM278 RNA, herein designated VGAM RNA, also designated SEQ ID:613.

Another function of VGAM278 is therefore inhibition of LOC149650 (Accession XM_{—}086623). Accordingly, utilities of VGAM278 include diagnosis, prevention and treatment of diseases and clinical conditions associated with LOC149650. LOC222223 (Accession XM_{—}168514) is another VGAM278 host target gene. LOC222223 BINDING SITE is HOST TARGET binding site found in the 3′ untranslated region of mRNA encoded by LOC222223, corresponding to a HOST TARGET binding site such as BINDING SITE I, BINDING SITE II or BINDING SITE III. Table 2 illustrates the complementarity of the nucleotide sequences of LOC222223 BINDING SITE, designated SEQ ID:3641, to the nucleotide sequence of VGAM278 RNA, herein designated VGAM RNA, also designated SEQ ID:613.

Another function of VGAM278 is therefore inhibition of LOC222223 (Accession XM_{—}168514). Accordingly, utilities of VGAM278 include diagnosis, prevention and treatment of diseases and clinical conditions associated with LOC222223. FIG. 1 further provides a conceptual description of a novel bioinformatically detected viral gene of the present invention, referred to here as Viral Genomic Address Messenger 279 (VGAM279) viral gene, which modulates expression of respective host target genes thereof, the function and utility of which host target genes is known in the art.

VGAM279 is a novel bioinformatically detected regulatory, non protein coding, viral micro RNA (miRNA) gene. The method by which VGAM279 was detected is described hereinabove with reference to FIGS. 1-8.

VGAM279 gene, herein designated VGAM GENE, is a viral gene contained in the genome of Vaccinia Virus. VGAM279 host target gene, herein designated VGAM HOST TARGET GENE, is a human gene contained in the human genome.

VGAM279 gene encodes a VGAM279 precursor RNA, herein designated VGAM PRECURSOR RNA. Similar to other miRNA genes, and unlike most ordinary genes, VGAM279 precursor RNA does not encode a protein. A nucleotide sequence identical or highly similar to the nucleotide sequence of VGAM279 precursor RNA is designated SEQ ID:265, and is provided hereinbelow with reference to the sequence listing part. Nucleotide sequence SEQ ID:265 is located at position 161106 relative to the genome of Vaccinia Virus.

VGAM279 precursor RNA folds onto itself, forming VGAM279 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM279 folded precursor RNA into VGAM279 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM279 RNA is designated SEQ ID:614, and is provided hereinbelow with reference to the sequence listing part.

VGAM279 host target gene, herein designated VGAM HOST TARGET GENE, encodes a corresponding messenger RNA, VGAM279 host target RNA, herein designated VGAM HOST TARGET RNA. VGAM279 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM279 RNA, herein designated VGAM RNA, binds complementarily to one or more host target binding sites located in untranslated regions of VGAM279 host target RNA, herein designated VGAM HOST TARGET RNA. This complementary binding is due to the fact that the nucleotide sequence of VGAM279 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM279 RNA, herein designated VGAM RNA, may have a different number of host target binding sites in untranslated regions of a VGAM279 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM279 RNA, herein designated VGAM RNA, to host target binding sites on VGAM279 host target RNA, herein designated VGAM HOST TARGET RNA, such as BINDING SITE I, BINDING SITE II and BINDING SITE III, inhibits translation of VGAM279 host target RNA into VGAM279 host target protein, herein designated VGAM HOST TARGET PROTEIN. VGAM host target protein is therefore outlined by a broken line.

It is appreciated that VGAM279 host target gene, herein designated VGAM HOST TARGET GENE, in fact represents a plurality of VGAM279 host target genes. The mRNA of each one of this plurality of VGAM279 host target genes comprises one or more host target binding sites, each having a nucleotide sequence which is at least partly complementary to VGAM279 RNA, herein designated VGAM RNA, and which when bound by VGAM279 RNA causes inhibition of translation of respective one or more VGAM279 host target proteins.

It is further appreciated by one skilled in the art that the mode of translational inhibition illustrated by FIG. 1 with specific reference to translational inhibition exerted by VGAM279 gene, herein designated VGAM GENE, on one or more VGAM279 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM279 is inhibition of expression of host target genes, as part of a novel viral mechanism of attacking a host. Accordingly, utilities of VGAM279 include diagnosis, prevention and treatment of viral infection by Vaccinia Virus. Specific functions, and accordingly utilities, of VGAM279 correlate with, and may be deduced from, the identity of the host target genes which VGAM279 binds and inhibits, and the function of these host target genes, as elaborated hereinbelow.

Nucleotide sequences of the VGAM279 precursor RNA, herein designated VGAM PRECURSOR RNA, and of the ‘diced’ VGAM279 RNA, herein designated VGAM RNA, and a schematic representation of the secondary folding of VGAM279 folded precursor RNA, herein designated VGAM FOLDED PRECURSOR RNA, of VGAM279 are further described hereinbelow with reference to Table 1.

Nucleotide sequences of host target binding sites, such as BINDING SITE-I, BINDING SITE-II and BINDING SITE-III of FIG. 1, found on VGAM279 host target RNA, and schematic representation of the complementarity of each of these host target binding sites to VGAM279 RNA, herein designated VGAM RNA, are described hereinbelow with reference to Table 2.

As mentioned hereinabove with reference to FIG. 1, a function of VGAM279 gene, herein designated VGAM is inhibition of expression of VGAM279 target genes. It is appreciated that specific functions, and accordingly utilities, of VGAM279 correlate with, and may be deduced from, the identity of the target genes which VGAM279 binds and inhibits, and the function of these target genes, as elaborated hereinbelow.

O-linked N-acetylglucosamine (GlcNAc) Transferase (UDP-N-acetylglucosamine:polypeptide-N-acetylglucosaminyl transferase) (OGT, Accession NM_{—}003605) is a VGAM279 host target gene. OGT BINDING SITE is HOST TARGET binding site found in the 5′ untranslated region of mRNA encoded by OGT, corresponding to a HOST TARGET binding site such as BINDING SITE I, BINDING SITE II or BINDING SITE III. Table 2 illustrates the complementarity of the nucleotide sequences of OGT BINDING SITE, designated SEQ ID: 1042, to the nucleotide sequence of VGAM279 RNA, herein designated VGAM RNA, also designated SEQ ID:614.

A function of VGAM279 is therefore inhibition of O-linked N-acetylglucosamine (GlcNAc) Transferase (UDP-N-acetylglucosamine:polypeptide-N-acetylglucosaminyl transferase) (OGT, Accession NM_{—}003605), a gene which has a role in the glycosylation of nuclear and cytoplasmic proteins. Accordingly, utilities of VGAM279 include diagnosis, prevention and treatment of diseases and clinical conditions associated with OGT. The function of OGT has been established by previous studies. O-linked N-acetylglucosamine (O-GlcNAc) transferase (OGT) catalyzes the addition of a single N-acetylglucosamine in O-glycosidic linkage to serine or threonine residues. Since both phosphorylation and glycosylation compete for similar serine or threonine residues, the 2 processes may compete for sites, or they may alter the substrate specificity of nearby sites by steric or electrostatic effects (Lubas et al., 1997). Haltiwanger et al. (1992) purified rat liver OGT and determined that it has a molecular mass of 340 kD. They proposed that OGT exists as a heterotrimeric complex with 2 subunits of 110 kD and 1 of 78 kD. However, using rabbit OGT, Lubas et al. (1997) analyzed the proteolytic fingerprint of both polypeptides and found that the 2 are related. They suggested that the 78 kD band is a proteolytic product of the 110 kD polypeptide or the product of an alternative translation start site. Kreppel et al. (1997) cloned rat cDNAs encoding the 110-kD subunit. Immunofluorescence of human cells expressing rat OGT indicated that OGT is present in both the nucleus and cytosol.

Full details of the abovementioned studies are described in the following publications, the disclosure of which are hereby incorporated by reference:
Kreppel, L. K.; Blomberg, M. A.; Hart, G. W.: Dynamic glycosylation of nuclear and cytosolic proteins: cloning and characterization of a unique O-GlcNAc transferase with multiple tetratricopeptide repeats. J. Biol. Chem. 272: 9308-9315, 1997.; and
Lubas, W. A.; Frank, D. W.; Krause, M.; Hanover, J. A.: O-linked GlcNAc transferase is a conserved nucleocytoplasmic protein containing tetratricopeptide repeats. J. Biol. Chem. 272: 9.

Further studies establishing the function and utilities of OGT are found in John Hopkins OMIM database record ID 300255, and in sited publications numbered 2032-2036 listed in the bibliography section hereinbelow, which are also hereby incorporated by reference. Tumor Necrosis Factor Receptor Super family, Member 10b (TNFRSF10B, Accession NM_{—}003842) is another VGAM279 host target gene. TNFRSF10B BINDING SITE is HOST TARGET binding site found in the 3′ untranslated region of mRNA encoded by TNFRSF10B, corresponding to a HOST TARGET binding site such as BINDING SITE I, BINDING SITE II or BINDING SITE III. Table 2 illustrates the complementarity of the nucleotide sequences of TNFRSF10B BINDING SITE, designated SEQ ID: 1065, to the nucleotide sequence of VGAM279 RNA, herein designated VGAM RNA, also designated SEQ ID:614.

Another function of VGAM279 is therefore inhibition of Tumor Necrosis Factor Receptor Super family, Member 10b (TNFRSF10B, Accession NM_{—}003842), a gene which forms complex that induces apoptosis. Accordingly, utilities of VGAM279 include diagnosis, prevention and treatment of diseases and clinical conditions associated with TNFRSF10B. The function of TNFRSF10B has been established by previous studies. The 8p21 region contains a number of putative tumor suppressor genes and is a frequent site of translocations in head and neck tumors. Pai et al. (1998) determined the genomic structure of KILLER/DR5 and performed sequence analysis of all 10 coding exons in 20 primary head and neck cancers with allelic loss of 8p. To screen for a subset of mutations localized to the functional cytoplasmic death domain, they sequenced this region in an additional 40 primary head and neck cancers. They found 2 alterations, including a 2-bp insertion at a minimal repeat site, introducing a premature stop codon and resulting in a truncated protein. This KILLER/DR5 mutation was also present in the germline of the affected patient, and the tumor did not have a p53 mutation by sequence analysis. Transfection studies in head and neck squamous cell carcinoma and colon and ovarian carcinoma cell lines revealed loss of growth suppressive function associated with the tumor-derived KILLER/DR5 truncation mutant. These observations provided the first evidence for mutation of a TRAIL death receptor gene in a human cancer, leading to loss of its apoptotic function. The second alteration identified by Pai et al. (1998) was a single T-to-C point mutation at residue 1109 that resulted in an amino acid change from val to ala. This mutation was not present in the germline; however, sequence analysis of p53 in this tumor revealed a point mutation of T to C in codon 242, resulting in a change from arg to cys. In a case of a head and neck squamous cell carcinoma (OMIM Ref. No. 601400), Pai et al. (1998) found a 2-bp insertion in the TNFRSF10B gene at a minimal repeat site (ACAC) at residue 1065, which introduced a premature stop codon and resulted in a truncated protein. Sequence analysis of normal tissue from the patient showed that the truncating mutation was also present in the germline, and that the tumor did not have a p53 mutation. A significant impairment in the ability of the truncation mutant to suppress colony formation was observed when mutant cDNA was transfected into human colon and ovarian cancer cell lines. In the wildtype transfected cells, there was no observed colony survival; however, there was more than 50% colony growth in cells transfected with the tumor-derived mutant. Pai et al. (1998) suspected that the mutant retained partial function, because its overexpression in a background of cells containing the endogenous wildtype gene could further reduce the percentage of colony survival.

Full details of the abovementioned studies are described in the following publications, the disclosure of which are hereby incorporated by reference:
Pai, S. I.; Wu, G. S.; Ozoren, N.; Wu, L.; Jen, J.; Sidransky, D.; El-Deiry, W. S.: Rare loss-of-function mutation of a death receptor gene in head and neck cancer. Cancer Res. 58: 3513-3518, 1998.; and
Screaton, G. R.; Mongkolsapaya, J.; Xu, X.-N.; Cowper, A. E.; McMichael, A. J.; Bell, J. I.: TRICK2, a new alternatively spliced receptor that transduces the cytotoxic signal from TRAIL.

Further studies establishing the function and utilities of TNFRSF10B are found in John Hopkins OMIM database record ID 603612, and in sited publications numbered 1395, 669-67 and 1723-1724 listed in the bibliography section hereinbelow, which are also hereby incorporated by reference. BA108L7.2 (Accession NM_{—}030971) is another VGAM279 host target gene. BA108L7.2 BINDING SITE is HOST TARGET binding site found in the 3′ untranslated region of mRNA encoded by BA108L7.2, corresponding to a HOST TARGET binding site such as BINDING SITE I, BINDING SITE II or BINDING SITE III. Table 2 illustrates the complementarity of the nucleotide sequences of BA108L7.2 BINDING SITE, designated SEQ ID:2180, to the nucleotide sequence of VGAM279 RNA, herein designated VGAM RNA, also designated SEQ ID:614.

Another function of VGAM279 is therefore inhibition of BA108L7.2 (Accession NM_{—}030971). Accordingly, utilities of VGAM279 include diagnosis, prevention and treatment of diseases and clinical conditions associated with BA108L7.2. FLJ12598 (Accession NM_{—}024754) is another VGAM279 host target gene. FLJ12598 BINDING SITE is HOST TARGET binding site found in the 5′ untranslated region of mRNA encoded by FLJ12598, corresponding to a HOST TARGET binding site such as BINDING SITE I, BINDING SITE II or BINDING SITE III. Table 2 illustrates the complementarity of the nucleotide sequences of FLJ12598 BINDING SITE, designated SEQ ID:2086, to the nucleotide sequence of VGAM279 RNA, herein designated VGAM RNA, also designated SEQ ID:614.

Another function of VGAM279 is therefore inhibition of FLJ12598 (Accession NM_{—}024754). Accordingly, utilities of VGAM279 include diagnosis, prevention and treatment of diseases and clinical conditions associated with FLJ12598. FLJ23071 (Accession NM_{—}025192) is another VGAM279 host target gene. FLJ23071 BINDING SITE is HOST TARGET binding site found in the 3′ untranslated region of mRNA encoded by FLJ23071, corresponding to a HOST TARGET binding site such as BINDING SITE I, BINDING SITE II or BINDING SITE III. Table 2 illustrates the complementarity of the nucleotide sequences of FLJ23071 BINDING SITE, designated SEQ ID:2144, to the nucleotide sequence of VGAM279 RNA, herein designated VGAM RNA, also designated SEQ ID:614.

Another function of VGAM279 is therefore inhibition of FLJ23071 (Accession NM_{—}025192). Accordingly, utilities of VGAM279 include diagnosis, prevention and treatment of diseases and clinical conditions associated with FLJ23071. LOC91266 (Accession XM_{—}037268) is another VGAM279 host target gene. LOC91266 BINDING SITE is HOST TARGET binding site found in the 5′ untranslated region of mRNA encoded by LOC91266, corresponding to a HOST TARGET binding site such as BINDING SITE I, BINDING SITE II or BINDING SITE III. Table 2 illustrates the complementarity of the nucleotide sequences of LOC91266 BINDING SITE, designated SEQ ID:2720, to the nucleotide sequence of VGAM279 RNA, herein designated VGAM RNA, also designated SEQ ID:614.

Another function of VGAM279 is therefore inhibition of LOC91266 (Accession XM_{—}037268). Accordingly, utilities of VGAM279 include diagnosis, prevention and treatment of diseases and clinical conditions associated with LOC91266. FIG. 1 further provides a conceptual description of a novel bioinformatically detected viral gene of the present invention, referred to here as Viral Genomic Address Messenger 280 (VGAM280) viral gene, which modulates expression of respective host target genes thereof, the function and utility of which host target genes is known in the art.

VGAM280 is a novel bioinformatically detected regulatory, non protein coding, viral micro RNA (miRNA) gene. The method by which VGAM280 was detected is described hereinabove with reference to FIGS. 1-8.

VGAM280 gene, herein designated VGAM GENE, is a viral gene contained in the genome of Vaccinia Virus. VGAM280 host target gene, herein designated VGAM HOST TARGET GENE, is a human gene contained in the human genome.

VGAM280 gene encodes a VGAM280 precursor RNA, herein designated VGAM PRECURSOR RNA. Similar to other miRNA genes, and unlike most ordinary genes, VGAM280 precursor RNA does not encode a protein. A nucleotide sequence identical or highly similar to the nucleotide sequence of VGAM280 precursor RNA is designated SEQ ID:266, and is provided hereinbelow with reference to the sequence listing part. Nucleotide sequence SEQ ID:266 is located at position 159680 relative to the genome of Vaccinia Virus.

VGAM280 precursor RNA folds onto itself, forming VGAM280 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM280 folded precursor RNA into VGAM280 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM280 RNA is designated SEQ ID:615, and is provided hereinbelow with reference to the sequence listing part.

VGAM280 host target gene, herein designated VGAM HOST TARGET GENE, encodes a corresponding messenger RNA, VGAM280 host target RNA, herein designated VGAM HOST TARGET RNA. VGAM280 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM280 RNA, herein designated VGAM RNA, binds complementarily to one or more host target binding sites located in untranslated regions of VGAM280 host target RNA, herein designated VGAM HOST TARGET RNA. This complementary binding is due to the fact that the nucleotide sequence of VGAM280 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM280 RNA, herein designated VGAM RNA, may have a different number of host target binding sites in untranslated regions of a VGAM280 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM280 RNA, herein designated VGAM RNA, to host target binding sites on VGAM280 host target RNA, herein designated VGAM HOST TARGET RNA, such as BINDING SITE I, BINDING SITE II and BINDING SITE III, inhibits translation of VGAM280 host target RNA into VGAM280 host target protein, herein designated VGAM HOST TARGET PROTEIN. VGAM host target protein is therefore outlined by a broken line.

It is appreciated that VGAM280 host target gene, herein designated VGAM HOST TARGET GENE, in fact represents a plurality of VGAM280 host target genes. The mRNA of each one of this plurality of VGAM280 host target genes comprises one or more host target binding sites, each having a nucleotide sequence which is at least partly complementary to VGAM280 RNA, herein designated VGAM RNA, and which when bound by VGAM280 RNA causes inhibition of translation of respective one or more VGAM280 host target proteins.

It is further appreciated by one skilled in the art that the mode of translational inhibition illustrated by FIG. 1 with specific reference to translational inhibition exerted by VGAM280 gene, herein designated VGAM GENE, on one or more VGAM280 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM280 is inhibition of expression of host target genes, as part of a novel viral mechanism of attacking a host. Accordingly, utilities of VGAM280 include diagnosis, prevention and treatment of viral infection by Vaccinia Virus. Specific functions, and accordingly utilities, of VGAM280 correlate with, and may be deduced from, the identity of the host target genes which VGAM280 binds and inhibits, and the function of these host target genes, as elaborated hereinbelow.

Nucleotide sequences of the VGAM280 precursor RNA, herein designated VGAM PRECURSOR RNA, and of the ‘diced’ VGAM280 RNA, herein designated VGAM RNA, and a schematic representation of the secondary folding of VGAM280 folded precursor RNA, herein designated VGAM FOLDED PRECURSOR RNA, of VGAM280 are further described hereinbelow with reference to Table 1.

Nucleotide sequences of host target binding sites, such as BINDING SITE-I, BINDING SITE-II and BINDING SITE-III of FIG. 1, found on VGAM280 host target RNA, and schematic representation of the complementarity of each of these host target binding sites to VGAM280 RNA, herein designated VGAM RNA, are described hereinbelow with reference to Table 2.

As mentioned hereinabove with reference to FIG. 1, a function of VGAM280 gene, herein designated VGAM is inhibition of expression of VGAM280 target genes. It is appreciated that specific functions, and accordingly utilities, of VGAM280 correlate with, and may be deduced from, the identity of the target genes which VGAM280 binds and inhibits, and the function of these target genes, as elaborated hereinbelow.

Cadherin 11, Type 2, OB-cadherin (osteoblast) (CDH11, Accession NM_{—}033664) is a VGAM280 host target gene. CDH11 BINDING SITE is HOST TARGET binding site found in the 5′ untranslated region of mRNA encoded by CDH11, corresponding to a HOST TARGET binding site such as BINDING SITE I, BINDING SITE II or BINDING SITE III. Table 2 illustrates the complementarity of the nucleotide sequences of CDH11 BINDING SITE, designated SEQ ID:2339, to the nucleotide sequence of VGAM280 RNA, herein designated VGAM RNA, also designated SEQ ID:615.

A function of VGAM280 is therefore inhibition of Cadherin 11, Type 2, OB-cadherin (osteoblast) (CDH11, Accession NM_{—}033664), a gene which plays an role in development and maintenance of tissues. Accordingly, utilities of VGAM280 include diagnosis, prevention and treatment of diseases and clinical conditions associated with CDH11. The function of CDH11 has been established by previous studies. Cadherins are a group of cell surface glycoproteins that mediate Ca(2+)-dependent cell-cell adhesion (Tanihara et al., 1994). These proteins have a molecular mass of about 120 kD and are composed of an extracellular domain at the N-terminal end and a relatively small cytoplasmic domain at the C-terminal end; the 2 domains are connected by a single membrane-spanning sequence. The extracellular domain consists of 5 subdomains, each of which contains a cadherin-specific motif. Cadherin expression is regulated spatially as well as temporally. Cadherins are thought to play an important role in development and maintenance of tissues through selective cell-cell adhesion activity and may be involved also in the invasion and metastasis of malignant tumors. Okazaki et al. (1994) identified a new member of the cadherin family, which they called OB (osteoblast)-cadherin, in a mouse osteoblastic cell line and human osteosarcoma. OB-cadherin was also weakly expressed in stroma cell lines that are the progenitor cells of osteoblasts. Expression of the CDHOB gene in the osteoblastic cell line was up-regulated during differentiation, suggesting a specific function in bone cell differentiation and bone formation. Two different forms of human OB-cadherin cDNA were cloned; one was a counterpart of the mouse gene and the other encoded a protein with a truncated cytoplasmic domain. The protein was designated cadherin-11 (symbol: CDH11) by the Human Genome Nomenclature Committee.

Full details of the abovementioned studies are described in the following publications, the disclosure of which are hereby incorporated by reference:
Kools, P.; Vanhalst, K.; Wan den Eynde, E.; van Roy, F.: The human cadherin-10 gene: complete coding sequence, predominant expression in the brain and mapping on chromosome 5p13-13. FEBS Lett. 452: 328-334, 1999.; and
Suzuki, S.; Sano, K.; Tanihara, H.: Diversity of the cadherin family: evidence for eight new cadherins in nervous tissue. Cell Regul. 2: 261-270, 1991.

Further studies establishing the function and utilities of CDH11 are found in John Hopkins OMIM database record ID 600023, and in sited publications numbered 190 and 2681-1905 listed in the bibliography section hereinbelow, which are also hereby incorporated by reference. Insulin-like Growth Factor 1 (somatomedin C) (IGF1, Accession NM_{—}000618) is another VGAM280 host target gene. IGF1 BINDING SITE is HOST TARGET binding site found in the 3′ untranslated region of mRNA encoded by IGF1, corresponding to a HOST TARGET binding site such as BINDING SITE I, BINDING SITE II or BINDING SITE III. Table 2 illustrates the complementarity of the nucleotide sequences of IGF1 BINDING SITE, designated SEQ ID:761, to the nucleotide sequence of VGAM280 RNA, herein designated VGAM RNA, also designated SEQ ID:615.

Another function of VGAM280 is therefore inhibition of Insulin-like Growth Factor 1 (somatomedin C) (IGF1, Accession NM_{—}000618), a gene which are structurally and functionally related to insulin but have a much higher growth-promoting activity. Accordingly, utilities of VGAM280 include diagnosis, prevention and treatment of diseases and clinical conditions associated with IGF1. The function of IGF1 and its association with various diseases and clinical conditions, has been established by previous studies, as described hereinabove with reference to VGAM34. FK506 Binding Protein 9, 63 KDa (FKBP9, Accession XM_{—}168403) is another VGAM280 host target gene. FKBP9 BINDING SITE is HOST TARGET binding site found in the 3′ untranslated region of mRNA encoded by FKBP9, corresponding to a HOST TARGET binding site such as BINDING SITE I, BINDING SITE II or BINDING SITE III. Table 2 illustrates the complementarity of the nucleotide sequences of FKBP9 BINDING SITE, designated SEQ ID:3632, to the nucleotide sequence of VGAM280 RNA, herein designated VGAM RNA, also designated SEQ ID:615.

Another function of VGAM280 is therefore inhibition of FK506 Binding Protein 9, 63 KDa (FKBP9, Accession XM_{—}168403). Accordingly, utilities of VGAM280 include diagnosis, prevention and treatment of diseases and clinical conditions associated with FKBP9. FLJ23045 (Accession NM_{—}024704) is another VGAM280 host target gene. FLJ23045 BINDING SITE is HOST TARGET binding site found in the 3′ untranslated region of mRNA encoded by FLJ23045, corresponding to a HOST TARGET binding site such as BINDING SITE I, BINDING SITE II or BINDING SITE III. Table 2 illustrates the complementarity of the nucleotide sequences of FLJ23045 BINDING SITE, designated SEQ ID:2082, to the nucleotide sequence of VGAM280 RNA, herein designated VGAM RNA, also designated SEQ ID:615.

Another function of VGAM280 is therefore inhibition of FLJ23045 (Accession NM_{—}024704). Accordingly, utilities of VGAM280 include diagnosis, prevention and treatment of diseases and clinical conditions associated with FLJ23045. FLJ30567 (Accession NM_{—}145022) is another VGAM280 host target gene. FLJ30567 BINDING SITE is HOST TARGET binding site found in the 3′ untranslated region of mRNA encoded by FLJ30567, corresponding to a HOST TARGET binding site such as BINDING SITE I, BINDING SITE II or BINDING SITE III. Table 2 illustrates the complementarity of the nucleotide sequences of FLJ30567 BINDING SITE, designated SEQ ID:2510, to the nucleotide sequence of VGAM280 RNA, herein designated VGAM RNA, also designated SEQ ID:615.

Another function of VGAM280 is therefore inhibition of FLJ30567 (Accession NM_{—}145022). Accordingly, utilities of VGAM280 include diagnosis, prevention and treatment of diseases and clinical conditions associated with FLJ30567. FLJ30678 (Accession NM_{—}144657) is another VGAM280 host target gene. FLJ30678 BINDING SITE is HOST TARGET binding site found in the 5′ untranslated region of mRNA encoded by FLJ30678, corresponding to a HOST TARGET binding site such as BINDING SITE I, BINDING SITE II or BINDING SITE III. Table 2 illustrates the complementarity of the nucleotide sequences of FLJ30678 BINDING SITE, designated SEQ ID:2490, to the nucleotide sequence of VGAM280 RNA, herein designated VGAM RNA, also designated SEQ ID:615.

Another function of VGAM280 is therefore inhibition of FLJ30678 (Accession NM_{—}144657). Accordingly, utilities of VGAM280 include diagnosis, prevention and treatment of diseases and clinical conditions associated with FLJ30678. KIAA1676 (Accession XM_{—}167612) is another VGAM280 host target gene. KIAA1676 BINDING SITE is HOST TARGET binding site found in the 3′ untranslated region of mRNA encoded by KIAA1676, corresponding to a HOST TARGET binding site such as BINDING SITE I, BINDING SITE II or BINDING SITE III. Table 2 illustrates the complementarity of the nucleotide sequences of KIAA1676 BINDING SITE, designated SEQ ID:3590, to the nucleotide sequence of VGAM280 RNA, herein designated VGAM RNA, also designated SEQ ID:615.

Another function of VGAM280 is therefore inhibition of KIAA1676 (Accession XM_{—}167612). Accordingly, utilities of VGAM280 include diagnosis, prevention and treatment of diseases and clinical conditions associated with KIAA1676. Kruppel-like Factor 3 (basic) (KLF3, Accession NM_{—}016531) is another VGAM280 host target gene. KLF3 BINDING SITE is HOST TARGET binding site found in the 3′ untranslated region of mRNA encoded by KLF3, corresponding to a HOST TARGET binding site such as BINDING SITE I, BINDING SITE II or BINDING SITE III. Table 2 illustrates the complementarity of the nucleotide sequences of KLF3 BINDING SITE, designated SEQ ID: 1693, to the nucleotide sequence of VGAM280 RNA, herein designated VGAM RNA, also designated SEQ ID:615.

Another function of VGAM280 is therefore inhibition of Kruppel-like Factor 3 (basic) (KLF3, Accession NM_{—}016531). Accordingly, utilities of VGAM280 include diagnosis, prevention and treatment of diseases and clinical conditions associated with KLF3. LOC146952 (Accession XM_{—}097138) is another VGAM280 host target gene. LOC146952 BINDING SITE is HOST TARGET binding site found in the 5′ untranslated region of mRNA encoded by LOC146952, corresponding to a HOST TARGET binding site such as BINDING SITE I, BINDING SITE II or BINDING SITE III. Table 2 illustrates the complementarity of the nucleotide sequences of LOC146952 BINDING SITE, designated SEQ ID:3286, to the nucleotide sequence of VGAM280 RNA, herein designated VGAM RNA, also designated SEQ ID:615.

Another function of VGAM280 is therefore inhibition of LOC146952 (Accession XM_{—}097138). Accordingly, utilities of VGAM280 include diagnosis, prevention and treatment of diseases and clinical conditions associated with LOC146952. LOC81569 (Accession XM_{—}030465) is another VGAM280 host target gene. LOC81569 BINDING SITE is HOST TARGET binding site found in the 3′ untranslated region of mRNA encoded by LOC81569, corresponding to a HOST TARGET binding site such as BINDING SITE I, BINDING SITE II or BINDING SITE III. Table 2 illustrates the complementarity of the nucleotide sequences of LOC81569 BINDING SITE, designated SEQ ID:2616, to the nucleotide sequence of VGAM280 RNA, herein designated VGAM RNA, also designated SEQ ID:615.

Another function of VGAM280 is therefore inhibition of LOC81569 (Accession XM_{—}030465). Accordingly, utilities of VGAM280 include diagnosis, prevention and treatment of diseases and clinical conditions associated with LOC81569. LOC83690 (Accession NM_{—}031461) is another VGAM280 host target gene. LOC83690 BINDING SITE is HOST TARGET binding site found in the 3′ untranslated region of mRNA encoded by LOC83690, corresponding to a HOST TARGET binding site such as BINDING SITE I, BINDING SITE II or BINDING SITE III. Table 2 illustrates the complementarity of the nucleotide sequences of LOC83690 BINDING SITE, designated SEQ ID:2204, to the nucleotide sequence of VGAM280 RNA, herein designated VGAM RNA, also designated SEQ ID:615.

Another function of VGAM280 is therefore inhibition of LOC83690 (Accession NM_{—}031461). Accordingly, utilities of VGAM280 include diagnosis, prevention and treatment of diseases and clinical conditions associated with LOC83690. FIG. 1 further provides a conceptual description of a novel bioinformatically detected viral gene of the present invention, referred to here as Viral Genomic Address Messenger 281 (VGAM281) viral gene, which modulates expression of respective host target genes thereof, the function and utility of which host target genes is known in the art.

VGAM281 is a novel bioinformatically detected regulatory, non protein coding, viral micro RNA (miRNA) gene. The method by which VGAM281 was detected is described hereinabove with reference to FIGS. 1-8.

VGAM281 gene, herein designated VGAM GENE, is a viral gene contained in the genome of Vaccinia Virus. VGAM281 host target gene, herein designated VGAM HOST TARGET GENE, is a human gene contained in the human genome.

VGAM281 gene encodes a VGAM281 precursor RNA, herein designated VGAM PRECURSOR RNA. Similar to other miRNA genes, and unlike most ordinary genes, VGAM281 precursor RNA does not encode a protein. A nucleotide sequence identical or highly similar to the nucleotide sequence of VGAM281 precursor RNA is designated SEQ ID:267, and is provided hereinbelow with reference to the sequence listing part. Nucleotide sequence SEQ ID:267 is located at position 160226 relative to the genome of Vaccinia Virus.

VGAM281 precursor RNA folds onto itself, forming VGAM281 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM281 folded precursor RNA into VGAM281 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM281 RNA is designated SEQ ID:616, and is provided hereinbelow with reference to the sequence listing part.

VGAM281 host target gene, herein designated VGAM HOST TARGET GENE, encodes a corresponding messenger RNA, VGAM281 host target RNA, herein designated VGAM HOST TARGET RNA. VGAM281 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM281 RNA, herein designated VGAM RNA, binds complementarily to one or more host target binding sites located in untranslated regions of VGAM281 host target RNA, herein designated VGAM HOST TARGET RNA. This complementary binding is due to the fact that the nucleotide sequence of VGAM281 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM281 RNA, herein designated VGAM RNA, may have a different number of host target binding sites in untranslated regions of a VGAM281 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM281 RNA, herein designated VGAM RNA, to host target binding sites on VGAM281 host target RNA, herein designated VGAM HOST TARGET RNA, such as BINDING SITE I, BINDING SITE II and BINDING SITE III, inhibits translation of VGAM281 host target RNA into VGAM281 host target protein, herein designated VGAM HOST TARGET PROTEIN. VGAM host target protein is therefore outlined by a broken line.

It is appreciated that VGAM281 host target gene, herein designated VGAM HOST TARGET GENE, in fact represents a plurality of VGAM281 host target genes. The mRNA of each one of this plurality of VGAM281 host target genes comprises one or more host target binding sites, each having a nucleotide sequence which is at least partly complementary to VGAM281 RNA, herein designated VGAM RNA, and which when bound by VGAM281 RNA causes inhibition of translation of respective one or more VGAM281 host target proteins.

It is further appreciated by one skilled in the art that the mode of translational inhibition illustrated by FIG. 1 with specific reference to translational inhibition exerted by VGAM281 gene, herein designated VGAM GENE, on one or more VGAM281 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM281 is inhibition of expression of host target genes, as part of a novel viral mechanism of attacking a host. Accordingly, utilities of VGAM281 include diagnosis, prevention and treatment of viral infection by Vaccinia Virus. Specific functions, and accordingly utilities, of VGAM281 correlate with, and may be deduced from, the identity of the host target genes which VGAM281 binds and inhibits, and the function of these host target genes, as elaborated hereinbelow.

Nucleotide sequences of the VGAM281 precursor RNA, herein designated VGAM PRECURSOR RNA, and of the ‘diced’ VGAM281 RNA, herein designated VGAM RNA, and a schematic representation of the secondary folding of VGAM281 folded precursor RNA, herein designated VGAM FOLDED PRECURSOR RNA, of VGAM281 are further described hereinbelow with reference to Table 1.

Nucleotide sequences of host target binding sites, such as BINDING SITE-I, BINDING SITE-II and BINDING SITE-III of FIG. 1, found on VGAM281 host target RNA, and schematic representation of the complementarity of each of these host target binding sites to VGAM281 RNA, herein designated VGAM RNA, are described hereinbelow with reference to Table 2.

As mentioned hereinabove with reference to FIG. 1, a function of VGAM281 gene, herein designated VGAM is inhibition of expression of VGAM281 target genes. It is appreciated that specific functions, and accordingly utilities, of VGAM281 correlate with, and may be deduced from, the identity of the target genes which VGAM281 binds and inhibits, and the function of these target genes, as elaborated hereinbelow.

Aspartate Beta-hydroxylase (ASPH, Accession NM_{—}020164) is a VGAM281 host target gene. ASPH BINDING SITE1 and ASPH BINDING SITE2 are HOST TARGET binding sites found in untranslated regions of mRNA encoded by ASPH, corresponding to HOST TARGET binding sites such as BINDING SITE I, BINDING SITE II or BINDING SITE III. Table 2 illustrates the complementarity of the nucleotide sequences of ASPH BINDING SITE1 and ASPH BINDING SITE2, designated SEQ ID:1896 and SEQ ID:2252 respectively, to the nucleotide sequence of VGAM281 RNA, herein designated VGAM RNA, also designated SEQ ID:616.

A function of VGAM281 is therefore inhibition of Aspartate Beta-hydroxylase (ASPH, Accession NM_{—}020164), a gene which specifically hydroxylates the beta carbon of aspartic acid or asparagine residues in certain epidermal growth factor (EGF)-like domains of a number of proteins. Accordingly, utilities of VGAM281 include diagnosis, prevention and treatment of diseases and clinical conditions associated with ASPH. The function of ASPH and its association with various diseases and clinical conditions, has been established by previous studies, as described hereinabove with reference to VGAM170. Solute Carrier Family 6 (neurotransmitter transporter, betaine/GABA), Member 12 (SLC6A12, Accession NM_{—}003044) is another VGAM281 host target gene. SLC6A12 BINDING SITE is HOST TARGET binding site found in the 5′ untranslated region of mRNA encoded by SLC6A12, corresponding to a HOST TARGET binding site such as BINDING SITE I, BINDING SITE II or BINDING SITE III. Table 2 illustrates the complementarity of the nucleotide sequences of SLC6A12 BINDING SITE, designated SEQ ID:987, to the nucleotide sequence of VGAM281 RNA, herein designated VGAM RNA, also designated SEQ ID:616.

Another function of VGAM281 is therefore inhibition of Solute Carrier Family 6 (neurotransmitter transporter, betaine/GABA), Member 12 (SLC6A12, Accession NM_{—}003044), a gene which transports betaine and gaba. Accordingly, utilities of VGAM281 include diagnosis, prevention and treatment of diseases and clinical conditions associated with SLC6A12. The function of SLC6A12 and its association with various diseases and clinical conditions, has been established by previous studies, as described hereinabove with reference to VGAM85. Cysteine Sulfinic Acid Decarboxylase (CSAD, Accession NM_{—}015989) is another VGAM281 host target gene. CSAD BINDING SITE is HOST TARGET binding site found in the 3′ untranslated region of mRNA encoded by CSAD, corresponding to a HOST TARGET binding site such as BINDING SITE I, BINDING SITE II or BINDING SITE III. Table 2 illustrates the complementarity of the nucleotide sequences of CSAD BINDING SITE, designated SEQ ID: 1654, to the nucleotide sequence of VGAM281 RNA, herein designated VGAM RNA, also designated SEQ ID:616.

Another function of VGAM281 is therefore inhibition of Cysteine Sulfinic Acid Decarboxylase (CSAD, Accession NM_{—}015989). Accordingly, utilities of VGAM281 include diagnosis, prevention and treatment of diseases and clinical conditions associated with CSAD. Dicer1, Dcr-1 Homolog (Drosophila) (DICER1, Accession NM_{—}030621) is another VGAM281 host target gene. DICER1 BINDING SITE is HOST TARGET binding site found in the 3′ untranslated region of mRNA encoded by DICER1, corresponding to a HOST TARGET binding site such as BINDING SITE I, BINDING SITE II or BINDING SITE III. Table 2 illustrates the complementarity of the nucleotide sequences of DICER1 BINDING SITE, designated SEQ ID:2151, to the nucleotide sequence of VGAM281 RNA, herein designated VGAM RNA, also designated SEQ ID:616.

Another function of VGAM281 is therefore inhibition of Dicer1, Dcr-1 Homolog (Drosophila) (DICER1, Accession NM_{—}030621). Accordingly, utilities of VGAM281 include diagnosis, prevention and treatment of diseases and clinical conditions associated with DICER1. KIAA1025 (Accession XM_{—}034056) is another VGAM281 host target gene. KIAA1025 BINDING SITE is HOST TARGET binding site found in the 3′ untranslated region of mRNA encoded by KIAA1025, corresponding to a HOST TARGET binding site such as BINDING SITE I, BINDING SITE II or BINDING SITE III. Table 2 illustrates the complementarity of the nucleotide sequences of KIAA1025 BINDING SITE, designated SEQ ID:2675, to the nucleotide sequence of VGAM281 RNA, herein designated VGAM RNA, also designated SEQ ID:616.

Another function of VGAM281 is therefore inhibition of KIAA1025 (Accession XM_{—}034056). Accordingly, utilities of VGAM281 include diagnosis, prevention and treatment of diseases and clinical conditions associated with KIAA1025. Kruppel-like Factor 12 (KLF12, Accession NM_{—}007249) is another VGAM281 host target gene. KLF12 BINDING SITE is HOST TARGET binding site found in the 3′ untranslated region of mRNA encoded by KLF12, corresponding to a HOST TARGET binding site such as BINDING SITE I, BINDING SITE II or BINDING SITE III. Table 2 illustrates the complementarity of the nucleotide sequences of KLF12 BINDING SITE, designated SEQ ID:1372, to the nucleotide sequence of VGAM281 RNA, herein designated VGAM RNA, also designated SEQ ID:616.

Another function of VGAM281 is therefore inhibition of Kruppel-like Factor 12 (KLF12, Accession NM_{—}007249). Accordingly, utilities of VGAM281 include diagnosis, prevention and treatment of diseases and clinical conditions associated with KLF12. Nuclear Factor of Activated T-cells 5, Tonicity-responsive (NFAT5, Accession NM_{—}138714) is another VGAM281 host target gene. NFAT5 BINDING SITE is HOST TARGET binding site found in the 3′ untranslated region of mRNA encoded by NFAT5, corresponding to a HOST TARGET binding site such as BINDING SITE I, BINDING SITE II or BINDING SITE III. Table 2 illustrates the complementarity of the nucleotide sequences of NFAT5 BINDING SITE, designated SEQ ID:2451, to the nucleotide sequence of VGAM281 RNA, herein designated VGAM RNA, also designated SEQ ID:616.

Another function of VGAM281 is therefore inhibition of Nuclear Factor of Activated T-cells 5, Tonicity-responsive (NFAT5, Accession NM_{—}138714). Accordingly, utilities of VGAM281 include diagnosis, prevention and treatment of diseases and clinical conditions associated with NFAT5. LOC170261 (Accession XM_{—}093214) is another VGAM281 host target gene. LOC170261 BINDING SITE is HOST TARGET binding site found in the 3′ untranslated region of mRNA encoded by LOC170261, corresponding to a HOST TARGET binding site such as BINDING SITE I, BINDING SITE II or BINDING SITE III. Table 2 illustrates the complementarity of the nucleotide sequences of LOC170261 BINDING SITE, designated SEQ ID:3256, to the nucleotide sequence of VGAM281 RNA, herein designated VGAM RNA, also designated SEQ ID:616.

Another function of VGAM281 is therefore inhibition of LOC170261 (Accession XM_{—}093214). Accordingly, utilities of VGAM281 include diagnosis, prevention and treatment of diseases and clinical conditions associated with LOC170261. LOC222602 (Accession XM_{—}167171) is another VGAM281 host target gene. LOC222602 BINDING SITE is HOST TARGET binding site found in the 3′ untranslated region of mRNA encoded by LOC222602, corresponding to a HOST TARGET binding site such as BINDING SITE I, BINDING SITE II or BINDING SITE III. Table 2 illustrates the complementarity of the nucleotide sequences of LOC222602 BINDING SITE, designated SEQ ID:3583, to the nucleotide sequence of VGAM281 RNA, herein designated VGAM RNA, also designated SEQ ID:616.

Another function of VGAM281 is therefore inhibition of LOC222602 (Accession XM_{—}167171). Accordingly, utilities of VGAM281 include diagnosis, prevention and treatment of diseases and clinical conditions associated with LOC222602. LOC257051 (Accession XM_{—}172800) is another VGAM281 host target gene. LOC257051 BINDING SITE is HOST TARGET binding site found in the 3′ untranslated region of mRNA encoded by LOC257051, corresponding to a HOST TARGET binding site such as BINDING SITE I, BINDING SITE II or BINDING SITE III. Table 2 illustrates the complementarity of the nucleotide sequences of LOC257051 BINDING SITE, designated SEQ ID:3703, to the nucleotide sequence of VGAM281 RNA, herein designated VGAM RNA, also designated SEQ ID:616.

Another function of VGAM281 is therefore inhibition of LOC257051 (Accession XM_{—}172800). Accordingly, utilities of VGAM281 include diagnosis, prevention and treatment of diseases and clinical conditions associated with LOC257051. FIG. 1 further provides a conceptual description of a novel bioinformatically detected viral gene of the present invention, referred to here as Viral Genomic Address Messenger 282 (VGAM282) viral gene, which modulates expression of respective host target genes thereof, the function and utility of which host target genes is known in the art.

VGAM282 is a novel bioinformatically detected regulatory, non protein coding, viral micro RNA (miRNA) gene. The method by which VGAM282 was detected is described hereinabove with reference to FIGS. 1-8.

VGAM282 gene, herein designated VGAM GENE, is a viral gene contained in the genome of Vaccinia Virus. VGAM282 host target gene, herein designated VGAM HOST TARGET GENE, is a human gene contained in the human genome.

VGAM282 gene encodes a VGAM282 precursor RNA, herein designated VGAM PRECURSOR RNA. Similar to other miRNA genes, and unlike most ordinary genes, VGAM282 precursor RNA does not encode a protein. A nucleotide sequence identical or highly similar to the nucleotide sequence of VGAM282 precursor RNA is designated SEQ ID:268, and is provided hereinbelow with reference to the sequence listing part. Nucleotide sequence SEQ ID:268 is located at position 161325 relative to the genome of Vaccinia Virus.

VGAM282 precursor RNA folds onto itself, forming VGAM282 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM282 folded precursor RNA into VGAM282 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM282 RNA is designated SEQ ID:617, and is provided hereinbelow with reference to the sequence listing part.

VGAM282 host target gene, herein designated VGAM HOST TARGET GENE, encodes a corresponding messenger RNA, VGAM282 host target RNA, herein designated VGAM HOST TARGET RNA. VGAM282 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM282 RNA, herein designated VGAM RNA, binds complementarily to one or more host target binding sites located in untranslated regions of VGAM282 host target RNA, herein designated VGAM HOST TARGET RNA. This complementary binding is due to the fact that the nucleotide sequence of VGAM282 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM282 RNA, herein designated VGAM RNA, may have a different number of host target binding sites in untranslated regions of a VGAM282 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM282 RNA, herein designated VGAM RNA, to host target binding sites on VGAM282 host target RNA, herein designated VGAM HOST TARGET RNA, such as BINDING SITE I, BINDING SITE II and BINDING SITE III, inhibits translation of VGAM282 host target RNA into VGAM282 host target protein, herein designated VGAM HOST TARGET PROTEIN. VGAM host target protein is therefore outlined by a broken line.

It is appreciated that VGAM282 host target gene, herein designated VGAM HOST TARGET GENE, in fact represents a plurality of VGAM282 host target genes. The mRNA of each one of this plurality of VGAM282 host target genes comprises one or more host target binding sites, each having a nucleotide sequence which is at least partly complementary to VGAM282 RNA, herein designated VGAM RNA, and which when bound by VGAM282 RNA causes inhibition of translation of respective one or more VGAM282 host target proteins.

It is further appreciated by one skilled in the art that the mode of translational inhibition illustrated by FIG. 1 with specific reference to translational inhibition exerted by VGAM282 gene, herein designated VGAM GENE, on one or more VGAM282 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM282 is inhibition of expression of host target genes, as part of a novel viral mechanism of attacking a host. Accordingly, utilities of VGAM282 include diagnosis, prevention and treatment of viral infection by Vaccinia Virus. Specific functions, and accordingly utilities, of VGAM282 correlate with, and may be deduced from, the identity of the host target genes which VGAM282 binds and inhibits, and the function of these host target genes, as elaborated hereinbelow.

Nucleotide sequences of the VGAM282 precursor RNA, herein designated VGAM PRECURSOR RNA, and of the ‘diced’ VGAM282 RNA, herein designated VGAM RNA, and a schematic representation of the secondary folding of VGAM282 folded precursor RNA, herein designated VGAM FOLDED PRECURSOR RNA, of VGAM282 are further described hereinbelow with reference to Table 1.

Nucleotide sequences of host target binding sites, such as BINDING SITE-I, BINDING SITE-II and BINDING SITE-III of FIG. 1, found on VGAM282 host target RNA, and schematic representation of the complementarity of each of these host target binding sites to VGAM282 RNA, herein designated VGAM RNA, are described hereinbelow with reference to Table 2.

As mentioned hereinabove with reference to FIG. 1, a function of VGAM282 gene, herein designated VGAM is inhibition of expression of VGAM282 target genes. It is appreciated that specific functions, and accordingly utilities, of VGAM282 correlate with, and may be deduced from, the identity of the target genes which VGAM282 binds and inhibits, and the function of these target genes, as elaborated hereinbelow.

Fibroblast Growth Factor 7 (keratinocyte growth factor) (FGF7, Accession NM_{—}002009) is a VGAM282 host target gene. FGF7 BINDING SITE is HOST TARGET binding site found in the 3′ untranslated region of mRNA encoded by FGF7, corresponding to a HOST TARGET binding site such as BINDING SITE I, BINDING SITE II or BINDING SITE III. Table 2 illustrates the complementarity of the nucleotide sequences of FGF7 BINDING SITE, designated SEQ ID:886, to the nucleotide sequence of VGAM282 RNA, herein designated VGAM RNA, also designated SEQ ID:617.

A function of VGAM282 is therefore inhibition of Fibroblast Growth Factor 7 (keratinocyte growth factor) (FGF7, Accession NM_{—}002009), a gene which growth factor active on keratinocytes. Accordingly, utilities of VGAM282 include diagnosis, prevention and treatment of diseases and clinical conditions associated with FGF7. The function of FGF7 has been established by previous studies. Rubin et al. (1989) identified a growth factor specific for epithelial cells in conditioned medium of a human embryonic lung fibroblast cell line. Because of its predominant activity in keratinocytes, it was referred to as keratinocyte growth factor. KGF was found to consist of a single polypeptide chain of about 28 kD. It was a potent mitogen for epithelial cells but lacked mitogenic activity on either fibroblasts or endothelial cells. Microsequencing showed an amino-terminal sequence containing no significant homology to any known protein. The release of this growth factor by human embryonic fibroblasts raised the possibility that KGF may play a role in mesenchymal stimulation of normal epithelial cell proliferation. In an addendum, Rubin et al. (1989) noted that by use of all the nucleotide probes based on the N-terminal sequence reported in their paper, they had isolated clones encoding KGF and had found significant structural homology between KGF and the other 5 known members of the fibroblast growth factor (FGF) family. Werner et al. (1994) assessed the function of KGF in normal and wounded skin by expression of a dominant-negative KGF receptor (OMIM Ref. No. 176943) in basal keratinocytes. The skin of transgenic mice was characterized by epidermal atrophy, abnormalities in the hair follicles, and dermal hyperthickening. Upon skin injury, inhibition of KGF receptor signaling reduced the proliferation rate of epidermal keratinocytes at the wound edge, resulting in substantially delayed reepithelialization of the wound. Using a cosmid probe encoding KGF exon 1 for fluorescence in situ hybridization, Zimonjic et al. (1997) assigned the KGF7 gene to 15q15-q21.1. In addition, copies of KGF-like sequences hybridizing only with a cosmid probe encoding exons 2 and 3 were localized to dispersed sites on chromosome 2q21, 9p11, 9q12-q13, 18p11, 18q11, 21q11, and 21q21.1. The distribution of KGF-like sequences suggested a role for alphoid DNA in their amplification and dispersion. In chimpanzee, KGF-like sequences were observed at 5 chromosomal sites, which were each homologous to sites in human, while in gorilla a subset of 4 of these homologous sites was identified. In orangutan 2 sites were identified, while gibbon exhibited only a single site. The chromosomal localization of KGF sequences in human and great ape genomes indicated that amplification and dispersion occurred in multiple discrete steps, with initial KGF gene duplication and dispersion occurring in multiple discrete steps, with initial KGF gene duplication and dispersion taking place in gibbon and involving loci corresponding to human chromosomes 15 and 21. The findings of Zimonjic et al. (1997) supported the concept of a closer evolutionary relationship of human with chimpanzee and with primates and a possible selective pressure for KGF dispersion during the evolution of higher primates.

Full details of the abovementioned studies are described in the following publications, the disclosure of which are hereby incorporated by reference:
Rubin, J. S.; Osada, H.; Finch, P. W.; Taylor, W. G.; Rudikoff, S.; Aaronson, S. A.: Purification and characterization of a newly identified growth factor specific for epithelial cells. Proc. Nat. Acad. Sci. 86: 802-806, 1989.; and
Werner, S.; Smola, H.; Liao, X.; Longaker, M. T.; Krieg, T.; Hofschneider, P. H.; Williams, L. T.: The function of KGF in morphogenesis of epithelium and reepithelialization of wounds.

Further studies establishing the function and utilities of FGF7 are found in John Hopkins OMIM database record ID 148180, and in sited publications numbered 2634-2638 listed in the bibliography section hereinbelow, which are also hereby incorporated by reference. Melatonin Receptor 1B (MTNR1B, Accession NM_{—}005959) is another VGAM282 host target gene. MTNR1B BINDING SITE is HOST TARGET binding site found in the 3′ untranslated region of mRNA encoded by MTNR1B, corresponding to a HOST TARGET binding site such as BINDING SITE I, BINDING SITE II or BINDING SITE III. Table 2 illustrates the complementarity of the nucleotide sequences of MTNR1B BINDING SITE, designated SEQ ID:1262, to the nucleotide sequence of VGAM282 RNA, herein designated VGAM RNA, also designated SEQ ID:617.

Another function of VGAM282 is therefore inhibition of Melatonin Receptor 1B (MTNR1B, Accession NM_{—}005959). Accordingly, utilities of VGAM282 include diagnosis, prevention and treatment of diseases and clinical conditions associated with MTNR1B. Selenoprotein X, 1 (SEPX1, Accession NM_{—}016332) is another VGAM282 host target gene. SEPX1 BINDING SITE is HOST TARGET binding site found in the 3′ untranslated region of mRNA encoded by SEPX1, corresponding to a HOST TARGET binding site such as BINDING SITE I, BINDING SITE II or BINDING SITE III. Table 2 illustrates the complementarity of the nucleotide sequences of SEPX1 BINDING SITE, designated SEQ ID:1681, to the nucleotide sequence of VGAM282 RNA, herein designated VGAM RNA, also designated SEQ ID:617.

Another function of VGAM282 is therefore inhibition of Selenoprotein X, 1 (SEPX1, Accession NM_{—}016332). Accordingly, utilities of VGAM282 include diagnosis, prevention and treatment of diseases and clinical conditions associated with SEPX1. Protein Tyrosine Phosphatase, Receptor Type, F Polypeptide (PTPRF), Interacting Protein (liprin), Alpha 4 (PPFIA4, Accession XM_{—}046751) is another VGAM282 host target gene. PPFIA4 BINDING SITE is HOST TARGET binding site found in the 3′ untranslated region of mRNA encoded by PPFIA4, corresponding to a HOST TARGET binding site such as BINDING SITE I, BINDING SITE II or BINDING SITE III. Table 2 illustrates the complementarity of the nucleotide sequences of PPFIA4 BINDING SITE, designated SEQ ID:2883, to the nucleotide sequence of VGAM282 RNA, herein designated VGAM RNA, also designated SEQ ID:617.

Another function of VGAM282 is therefore inhibition of Protein Tyrosine Phosphatase, Receptor Type, F Polypeptide (PTPRF), Interacting Protein (liprin), Alpha 4 (PPFIA4, Accession XM_{—}046751). Accordingly, utilities of VGAM282 include diagnosis, prevention and treatment of diseases and clinical conditions associated with PPFIA4. LOC145231 (Accession XM_{—}096740) is another VGAM282 host target gene. LOC145231 BINDING SITE is HOST TARGET binding site found in the 3′ untranslated region of mRNA encoded by LOC145231, corresponding to a HOST TARGET binding site such as BINDING SITE I, BINDING SITE II or BINDING SITE III. Table 2 illustrates the complementarity of the nucleotide sequences of LOC145231 BINDING SITE, designated SEQ ID:3274, to the nucleotide sequence of VGAM282 RNA, herein designated VGAM RNA, also designated SEQ ID:617.

Another function of VGAM282 is therefore inhibition of LOC145231 (Accession XM_{—}096740). Accordingly, utilities of VGAM282 include diagnosis, prevention and treatment of diseases and clinical conditions associated with LOC145231. FIG. 1 further provides a conceptual description of a novel bioinformatically detected viral gene of the present invention, referred to here as Viral Genomic Address Messenger 283 (VGAM283) viral gene, which modulates expression of respective host target genes thereof, the function and utility of which host target genes is known in the art.

VGAM283 is a novel bioinformatically detected regulatory, non protein coding, viral micro RNA (miRNA) gene. The method by which VGAM283 was detected is described hereinabove with reference to FIGS. 1-8.

VGAM283 gene, herein designated VGAM GENE, is a viral gene contained in the genome of Vaccinia Virus. VGAM283 host target gene, herein designated VGAM HOST TARGET GENE, is a human gene contained in the human genome.

VGAM283 gene encodes a VGAM283 precursor RNA, herein designated VGAM PRECURSOR RNA. Similar to other miRNA genes, and unlike most ordinary genes, VGAM283 precursor RNA does not encode a protein. A nucleotide sequence identical or highly similar to the nucleotide sequence of VGAM283 precursor RNA is designated SEQ ID:269, and is provided hereinbelow with reference to the sequence listing part. Nucleotide sequence SEQ ID:269 is located at position 158874 relative to the genome of Vaccinia Virus.

VGAM283 precursor RNA folds onto itself, forming VGAM283 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM283 folded precursor RNA into VGAM283 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM283 RNA is designated SEQ ID:618, and is provided hereinbelow with reference to the sequence listing part.

VGAM283 host target gene, herein designated VGAM HOST TARGET GENE, encodes a corresponding messenger RNA, VGAM283 host target RNA, herein designated VGAM HOST TARGET RNA. VGAM283 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM283 RNA, herein designated VGAM RNA, binds complementarily to one or more host target binding sites located in untranslated regions of VGAM283 host target RNA, herein designated VGAM HOST TARGET RNA. This complementary binding is due to the fact that the nucleotide sequence of VGAM283 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM283 RNA, herein designated VGAM RNA, may have a different number of host target binding sites in untranslated regions of a VGAM283 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM283 RNA, herein designated VGAM RNA, to host target binding sites on VGAM283 host target RNA, herein designated VGAM HOST TARGET RNA, such as BINDING SITE I, BINDING SITE II and BINDING SITE III, inhibits translation of VGAM283 host target RNA into VGAM283 host target protein, herein designated VGAM HOST TARGET PROTEIN. VGAM host target protein is therefore outlined by a broken line.

It is appreciated that VGAM283 host target gene, herein designated VGAM HOST TARGET GENE, in fact represents a plurality of VGAM283 host target genes. The mRNA of each one of this plurality of VGAM283 host target genes comprises one or more host target binding sites, each having a nucleotide sequence which is at least partly complementary to VGAM283 RNA, herein designated VGAM RNA, and which when bound by VGAM283 RNA causes inhibition of translation of respective one or more VGAM283 host target proteins.

It is further appreciated by one skilled in the art that the mode of translational inhibition illustrated by FIG. 1 with specific reference to translational inhibition exerted by VGAM283 gene, herein designated VGAM GENE, on one or more VGAM283 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM283 is inhibition of expression of host target genes, as part of a novel viral mechanism of attacking a host. Accordingly, utilities of VGAM283 include diagnosis, prevention and treatment of viral infection by Vaccinia Virus. Specific functions, and accordingly utilities, of VGAM283 correlate with, and may be deduced from, the identity of the host target genes which VGAM283 binds and inhibits, and the function of these host target genes, as elaborated hereinbelow.

Nucleotide sequences of the VGAM283 precursor RNA, herein designated VGAM PRECURSOR RNA, and of the ‘diced’ VGAM283 RNA, herein designated VGAM RNA, and a schematic representation of the secondary folding of VGAM283 folded precursor RNA, herein designated VGAM FOLDED PRECURSOR RNA, of VGAM283 are further described hereinbelow with reference to Table 1.

Nucleotide sequences of host target binding sites, such as BINDING SITE-I, BINDING SITE-II and BINDING SITE-III of FIG. 1, found on VGAM283 host target RNA, and schematic representation of the complementarity of each of these host target binding sites to VGAM283 RNA, herein designated VGAM RNA, are described hereinbelow with reference to Table 2.

As mentioned hereinabove with reference to FIG. 1, a function of VGAM283 gene, herein designated VGAM is inhibition of expression of VGAM283 target genes. It is appreciated that specific functions, and accordingly utilities, of VGAM283 correlate with, and may be deduced from, the identity of the target genes which VGAM283 binds and inhibits, and the function of these target genes, as elaborated hereinbelow.

Caveolin 3 (CAV3, Accession NM_{—}033337) is a VGAM283 host target gene. CAV3 BINDING SITE is HOST TARGET binding site found in the 3′ untranslated region of mRNA encoded by CAV3, corresponding to a HOST TARGET binding site such as BINDING SITE I, BINDING SITE II or BINDING SITE III. Table 2 illustrates the complementarity of the nucleotide sequences of CAV3 BINDING SITE, designated SEQ ID:2329, to the nucleotide sequence of VGAM283 RNA, herein designated VGAM RNA, also designated SEQ ID:618.

A function of VGAM283 is therefore inhibition of Caveolin 3 (CAV3, Accession NM_{—}033337). Accordingly, utilities of VGAM283 include diagnosis, prevention and treatment of diseases and clinical conditions associated with CAV3. Homeo Box C4 (HOXC4, Accession NM_{—}014620) is another VGAM283 host target gene. HOXC4 BINDING SITE is HOST TARGET binding site found in the 5′ untranslated region of mRNA encoded by HOXC4, corresponding to a HOST TARGET binding site such as BINDING SITE I, BINDING SITE II or BINDING SITE III. Table 2 illustrates the complementarity of the nucleotide sequences of HOXC4 BINDING SITE, designated SEQ ID:1509, to the nucleotide sequence of VGAM283 RNA, herein designated VGAM RNA, also designated SEQ ID:618.

Another function of VGAM283 is therefore inhibition of Homeo Box C4 (HOXC4, Accession NM_{—}014620), a gene which is part of a developmental regulatory system that provides cells with specific positional identities on the anterior-posterior axis. Accordingly, utilities of VGAM283 include diagnosis, prevention and treatment of diseases and clinical conditions associated with HOXC4. The function of HOXC4 has been established by previous studies. As reviewed by Acampora et al. (1989), the homeo box region 3, which maps to 12q12-q13, contains at least 7 homeo boxes in 160 kb of DNA. HOX3A is homologous to mouse Hox-3.1; HOX3B to mouse Hox-3.2; HOX3C to mouse Hox-6.1, and HOX3D to mouse Hox-6.2. The order of genes, from 5-prime to 3-prime, is HOX3G, HOX3F, HOX3B, HOX3A, HOX3C, HOX3D, HOX3E (Acampora et al., 1989). Masuda et al. (1991) mapped the feline equivalent to chromosome B4, which shares syntenic homology with human chromosome 12 and mouse chromosome 15. This gene is also called HOXC8; see HOXC9 (OMIM Ref. No. 142971). Yueh et al. (1998) showed that overexpression of a Hoxc8 transgene causes cartilage defects whose severity depends on transgene dosage. The abnormal cartilage is characterized by an accumulation of proliferating chondrocytes and reduced maturation. Since Hoxc8 is normally expressed in chondrocytes, these results suggested that Hoxc8 continues to regulate skeletal development well beyond pattern formation in a tissue-specific manner, presumably by controlling the progression of cells along the chondrocyte differentiation pathway. They found that Hoxd4 and Hoxc8 appear to act on chondrocyte differentiation in a similar manner. The protein sequences of the 2 share 67% identity within the homeodomain and 50% in the hexapeptide motif but little similarity in the remaining 70% of the molecules. Isl1, which shares no significant sequence similarities with Hoxc8 or Hoxd4, is not associated with abnormalities of skeletal development, implying that the cartilage abnormalities are specifically induced by HOX genes. The capacity of the HOX genes to regulate cartilage differentiation suggests that they may be involved in human chondrodysplasias or other cartilage disorders.

Full details of the abovementioned studies are described in the following publications, the disclosure of which are hereby incorporated by reference:
Simeone, A.; Pannese, M.; Acampora, D.; D'Esposito, M.; Boncinelli, E.: At least three human homeoboxes on chromosome 12 belong to the same transcription unit. Nucleic Acids Res. 16: 5379-5390, 1988.; and
Yueh, Y. G.; Gardner, D. P.; Kappen, C.: Evidence for regulation of cartilage differentiation by the homeobox gene Hoxc-8. Proc. Nat. Acad. Sci. 95: 9956-9961, 1998.

Further studies establishing the function and utilities of HOXC4 are found in John Hopkins OMIM database record ID 142974, and in sited publications numbered 1189 listed in the bibliography section hereinbelow, which are also hereby incorporated by reference. V-myc Myelocytomatosis Viral Oncogene Homolog 1, Lung Carcinoma Derived (avian) (MYCL1, Accession NM_{—}005376) is another VGAM283 host target gene. MYCL1 BINDING SITE is HOST TARGET binding site found in the 3′ untranslated region of mRNA encoded by MYCL1, corresponding to a HOST TARGET binding site such as BINDING SITE I, BINDING SITE II or BINDING SITE III. Table 2 illustrates the complementarity of the nucleotide sequences of MYCL1 BINDING SITE, designated SEQ ID:1203, to the nucleotide sequence of VGAM283 RNA, herein designated VGAM RNA, also designated SEQ ID:618.

Another function of VGAM283 is therefore inhibition of V-myc Myelocytomatosis Viral Oncogene Homolog 1, Lung Carcinoma Derived (avian) (MYCL1, Accession NM_{—}005376), a gene which is a Myc-like transcription factor. Accordingly, utilities of VGAM283 include diagnosis, prevention and treatment of diseases and clinical conditions associated with MYCL1. The function of MYCL1 and its association with various diseases and clinical conditions, has been established by previous studies, as described hereinabove with reference to VGAM152. FLJ20666 (Accession NM_{—}017922) is another VGAM283 host target gene. FLJ20666 BINDING SITE is HOST TARGET binding site found in the 5′ untranslated region of mRNA encoded by FLJ20666, corresponding to a HOST TARGET binding site such as BINDING SITE I, BINDING SITE II or BINDING SITE III. Table 2 illustrates the complementarity of the nucleotide sequences of FLJ20666 BINDING SITE, designated SEQ ID: 1762, to the nucleotide sequence of VGAM283 RNA, herein designated VGAM RNA, also designated SEQ ID:618.

Another function of VGAM283 is therefore inhibition of FLJ20666 (Accession NM_{—}017922). Accordingly, utilities of VGAM283 include diagnosis, prevention and treatment of diseases and clinical conditions associated with FLJ20666. KIAA1001 (Accession NM_{—}014960) is another VGAM283 host target gene. KIAA1001 BINDING SITE is HOST TARGET binding site found in the 3′ untranslated region of mRNA encoded by KIAA1001, corresponding to a HOST TARGET binding site such as BINDING SITE I, BINDING SITE II or BINDING SITE III. Table 2 illustrates the complementarity of the nucleotide sequences of KIAA1001 BINDING SITE, designated SEQ ID:1599, to the nucleotide sequence of VGAM283 RNA, herein designated VGAM RNA, also designated SEQ ID:618.

Another function of VGAM283 is therefore inhibition of KIAA1001 (Accession NM_{—}014960). Accordingly, utilities of VGAM283 include diagnosis, prevention and treatment of diseases and clinical conditions associated with KIAA1001. Progesterone Receptor Membrane Component 2 (PGRMC2, Accession NM_{—}006320) is another VGAM283 host target gene. PGRMC2 BINDING SITE is HOST TARGET binding site found in the 3′ untranslated region of mRNA encoded by PGRMC2, corresponding to a HOST TARGET binding site such as BINDING SITE I, BINDING SITE II or BINDING SITE III. Table 2 illustrates the complementarity of the nucleotide sequences of PGRMC2 BINDING SITE, designated SEQ ID:1286, to the nucleotide sequence of VGAM283 RNA, herein designated VGAM RNA, also designated SEQ ID:618.

Another function of VGAM283 is therefore inhibition of Progesterone Receptor Membrane Component 2 (PGRMC2, Accession NM_{—}006320). Accordingly, utilities of VGAM283 include diagnosis, prevention and treatment of diseases and clinical conditions associated with PGRMC2. LOC152286 (Accession XM_{—}098188) is another VGAM283 host target gene. LOC152286 BINDING SITE is HOST TARGET binding site found in the 3′ untranslated region of mRNA encoded by LOC152286, corresponding to a HOST TARGET binding site such as BINDING SITE I, BINDING SITE II or BINDING SITE III. Table 2 illustrates the complementarity of the nucleotide sequences of LOC152286 BINDING SITE, designated SEQ ID:3354, to the nucleotide sequence of VGAM283 RNA, herein designated VGAM RNA, also designated SEQ ID:618.

Another function of VGAM283 is therefore inhibition of LOC152286 (Accession XM_{—}098188). Accordingly, utilities of VGAM283 include diagnosis, prevention and treatment of diseases and clinical conditions associated with LOC152286. LOC158549 (Accession XM_{—}098963) is another VGAM283 host target gene. LOC158549 BINDING SITE is HOST TARGET binding site found in the 3′ untranslated region of mRNA encoded by LOC158549, corresponding to a HOST TARGET binding site such as BINDING SITE I, BINDING SITE II or BINDING SITE III. Table 2 illustrates the complementarity of the nucleotide sequences of LOC158549 BINDING SITE, designated SEQ ID:3392, to the nucleotide sequence of VGAM283 RNA, herein designated VGAM RNA, also designated SEQ ID:618.

Another function of VGAM283 is therefore inhibition of LOC158549 (Accession XM_{—}098963). Accordingly, utilities of VGAM283 include diagnosis, prevention and treatment of diseases and clinical conditions associated with LOC158549. FIG. 1 further provides a conceptual description of a novel bioinformatically detected viral gene of the present invention, referred to here as Viral Genomic Address Messenger 284 (VGAM284) viral gene, which modulates expression of respective host target genes thereof, the function and utility of which host target genes is known in the art.

VGAM284 is a novel bioinformatically detected regulatory, non protein coding, viral micro RNA (miRNA) gene. The method by which VGAM284 was detected is described hereinabove with reference to FIGS. 1-8.

VGAM284 gene, herein designated VGAM GENE, is a viral gene contained in the genome of Vaccinia Virus. VGAM284 host target gene, herein designated VGAM HOST TARGET GENE, is a human gene contained in the human genome.

VGAM284 gene encodes a VGAM284 precursor RNA, herein designated VGAM PRECURSOR RNA. Similar to other miRNA genes, and unlike most ordinary genes, VGAM284 precursor RNA does not encode a protein. A nucleotide sequence identical or highly similar to the nucleotide sequence of VGAM284 precursor RNA is designated SEQ ID:270, and is provided hereinbelow with reference to the sequence listing part. Nucleotide sequence SEQ ID:270 is located at position 159230 relative to the genome of Vaccinia Virus.

VGAM284 precursor RNA folds onto itself, forming VGAM284 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM284 folded precursor RNA into VGAM284 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM284 RNA is designated SEQ ID:619, and is provided hereinbelow with reference to the sequence listing part.

VGAM284 host target gene, herein designated VGAM HOST TARGET GENE, encodes a corresponding messenger RNA, VGAM284 host target RNA, herein designated VGAM HOST TARGET RNA. VGAM284 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM284 RNA, herein designated VGAM RNA, binds complementarily to one or more host target binding sites located in untranslated regions of VGAM284 host target RNA, herein designated VGAM HOST TARGET RNA. This complementary binding is due to the fact that the nucleotide sequence of VGAM284 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM284 RNA, herein designated VGAM RNA, may have a different number of host target binding sites in untranslated regions of a VGAM284 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM284 RNA, herein designated VGAM RNA, to host target binding sites on VGAM284 host target RNA, herein designated VGAM HOST TARGET RNA, such as BINDING SITE I, BINDING SITE II and BINDING SITE III, inhibits translation of VGAM284 host target RNA into VGAM284 host target protein, herein designated VGAM HOST TARGET PROTEIN. VGAM host target protein is therefore outlined by a broken line.

It is appreciated that VGAM284 host target gene, herein designated VGAM HOST TARGET GENE, in fact represents a plurality of VGAM284 host target genes. The mRNA of each one of this plurality of VGAM284 host target genes comprises one or more host target binding sites, each having a nucleotide sequence which is at least partly complementary to VGAM284 RNA, herein designated VGAM RNA, and which when bound by VGAM284 RNA causes inhibition of translation of respective one or more VGAM284 host target proteins.

It is further appreciated by one skilled in the art that the mode of translational inhibition illustrated by FIG. 1 with specific reference to translational inhibition exerted by VGAM284 gene, herein designated VGAM GENE, on one or more VGAM284 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM284 is inhibition of expression of host target genes, as part of a novel viral mechanism of attacking a host. Accordingly, utilities of VGAM284 include diagnosis, prevention and treatment of viral infection by Vaccinia Virus. Specific functions, and accordingly utilities, of VGAM284 correlate with, and may be deduced from, the identity of the host target genes which VGAM284 binds and inhibits, and the function of these host target genes, as elaborated hereinbelow.

Nucleotide sequences of the VGAM284 precursor RNA, herein designated VGAM PRECURSOR RNA, and of the ‘diced’ VGAM284 RNA, herein designated VGAM RNA, and a schematic representation of the secondary folding of VGAM284 folded precursor RNA, herein designated VGAM FOLDED PRECURSOR RNA, of VGAM284 are further described hereinbelow with reference to Table 1.

Nucleotide sequences of host target binding sites, such as BINDING SITE-I, BINDING SITE-II and BINDING SITE-III of FIG. 1, found on VGAM284 host target RNA, and schematic representation of the complementarity of each of these host target binding sites to VGAM284 RNA, herein designated VGAM RNA, are described hereinbelow with reference to Table 2.

As mentioned hereinabove with reference to FIG. 1, a function of VGAM284 gene, herein designated VGAM is inhibition of expression of VGAM284 target genes. It is appreciated that specific functions, and accordingly utilities, of VGAM284 correlate with, and may be deduced from, the identity of the target genes which VGAM284 binds and inhibits, and the function of these target genes, as elaborated hereinbelow.

DKFZp761G0313 (Accession XM_{—}038026) is a VGAM284 host target gene. DKFZp761G0313 BINDING SITE is HOST TARGET binding site found in the 3′ untranslated region of mRNA encoded by DKFZp761G0313, corresponding to a HOST TARGET binding site such as BINDING SITE I, BINDING SITE II or BINDING SITE III. Table 2 illustrates the complementarity of the nucleotide sequences of DKFZp761G0313 BINDING SITE, designated SEQ ID:2731, to the nucleotide sequence of VGAM284 RNA, herein designated VGAM RNA, also designated SEQ ID:619.

A function of VGAM284 is therefore inhibition of DKFZp761G0313 (Accession XM_{—}038026). Accordingly, utilities of VGAM284 include diagnosis, prevention and treatment of diseases and clinical conditions associated with DKFZp761G0313. FIG. 1 further provides a conceptual description of a novel bioinformatically detected viral gene of the present invention, referred to here as Viral Genomic Address Messenger 285 (VGAM285) viral gene, which modulates expression of respective host target genes thereof, the function and utility of which host target genes is known in the art.

VGAM285 is a novel bioinformatically detected regulatory, non protein coding, viral micro RNA (miRNA) gene. The method by which VGAM285 was detected is described hereinabove with reference to FIGS. 1-8.

VGAM285 gene, herein designated VGAM GENE, is a viral gene contained in the genome of Vaccinia Virus. VGAM285 host target gene, herein designated VGAM HOST TARGET GENE, is a human gene contained in the human genome.

VGAM285 gene encodes a VGAM285 precursor RNA, herein designated VGAM PRECURSOR RNA. Similar to other miRNA genes, and unlike most ordinary genes, VGAM285 precursor RNA does not encode a protein. A nucleotide sequence identical or highly similar to the nucleotide sequence of VGAM285 precursor RNA is designated SEQ ID:271, and is provided hereinbelow with reference to the sequence listing part. Nucleotide sequence SEQ ID:271 is located at position 163176 relative to the genome of Vaccinia Virus.

VGAM285 precursor RNA folds onto itself, forming VGAM285 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM285 folded precursor RNA into VGAM285 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM285 RNA is designated SEQ ID:620, and is provided hereinbelow with reference to the sequence listing part.

VGAM285 host target gene, herein designated VGAM HOST TARGET GENE, encodes a corresponding messenger RNA, VGAM285 host target RNA, herein designated VGAM HOST TARGET RNA. VGAM285 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM285 RNA, herein designated VGAM RNA, binds complementarily to one or more host target binding sites located in untranslated regions of VGAM285 host target RNA, herein designated VGAM HOST TARGET RNA. This complementary binding is due to the fact that the nucleotide sequence of VGAM285 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM285 RNA, herein designated VGAM RNA, may have a different number of host target binding sites in untranslated regions of a VGAM285 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM285 RNA, herein designated VGAM RNA, to host target binding sites on VGAM285 host target RNA, herein designated VGAM HOST TARGET RNA, such as BINDING SITE I, BINDING SITE II and BINDING SITE III, inhibits translation of VGAM285 host target RNA into VGAM285 host target protein, herein designated VGAM HOST TARGET PROTEIN. VGAM host target protein is therefore outlined by a broken line.

It is appreciated that VGAM285 host target gene, herein designated VGAM HOST TARGET GENE, in fact represents a plurality of VGAM285 host target genes. The mRNA of each one of this plurality of VGAM285 host target genes comprises one or more host target binding sites, each having a nucleotide sequence which is at least partly complementary to VGAM285 RNA, herein designated VGAM RNA, and which when bound by VGAM285 RNA causes inhibition of translation of respective one or more VGAM285 host target proteins.

It is further appreciated by one skilled in the art that the mode of translational inhibition illustrated by FIG. 1 with specific reference to translational inhibition exerted by VGAM285 gene, herein designated VGAM GENE, on one or more VGAM285 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM285 is inhibition of expression of host target genes, as part of a novel viral mechanism of attacking a host. Accordingly, utilities of VGAM285 include diagnosis, prevention and treatment of viral infection by Vaccinia Virus. Specific functions, and accordingly utilities, of VGAM285 correlate with, and may be deduced from, the identity of the host target genes which VGAM285 binds and inhibits, and the function of these host target genes, as elaborated hereinbelow.

Nucleotide sequences of the VGAM285 precursor RNA, herein designated VGAM PRECURSOR RNA, and of the ‘diced’ VGAM285 RNA, herein designated VGAM RNA, and a schematic representation of the secondary folding of VGAM285 folded precursor RNA, herein designated VGAM FOLDED PRECURSOR RNA, of VGAM285 are further described hereinbelow with reference to Table 1.

Nucleotide sequences of host target binding sites, such as BINDING SITE-I, BINDING SITE-II and BINDING SITE-III of FIG. 1, found on VGAM285 host target RNA, and schematic representation of the complementarity of each of these host target binding sites to VGAM285 RNA, herein designated VGAM RNA, are described hereinbelow with reference to Table 2.

As mentioned hereinabove with reference to FIG. 1, a function of VGAM285 gene, herein designated VGAM is inhibition of expression of VGAM285 target genes. It is appreciated that specific functions, and accordingly utilities, of VGAM285 correlate with, and may be deduced from, the identity of the target genes which VGAM285 binds and inhibits, and the function of these target genes, as elaborated hereinbelow.

Neuronal Cell Adhesion Molecule (NRCAM, Accession NM_{—}005010) is a VGAM285 host target gene. NRCAM BINDING SITE is HOST TARGET binding site found in the 3′ untranslated region of mRNA encoded by NRCAM, corresponding to a HOST TARGET binding site such as BINDING SITE I, BINDING SITE II or BINDING SITE III. Table 2 illustrates the complementarity of the nucleotide sequences of NRCAM BINDING SITE, designated SEQ ID:1170, to the nucleotide sequence of VGAM285 RNA, herein designated VGAM RNA, also designated SEQ ID:620.

A function of VGAM285 is therefore inhibition of Neuronal Cell Adhesion Molecule (NRCAM, Accession NM_{—}005010), a gene which functions as a cell surface protein and belongs to the immunoglobulin super family. Accordingly, utilities of VGAM285 include diagnosis, prevention and treatment of diseases and clinical conditions associated with NRCAM. The function of NRCAM has been established by previous studies. The cell adhesion molecules (CAMs) are a subset of the immunoglobin (Ig) super family found in the nervous systems of both vertebrates and invertebrates. They are usually surface membrane proteins with multiple Ig domains at their N termini followed by several fibronectin type III repeats and either a transmembrane intracellular domain or a glycophosphatidylinositol-linked membrane anchor at the C terminus (Lane et al., 1996). The chicken Bravo/Nr-CAM was described by Grumet et al. (1991) and Kayyem et al. (1992) and shown to play a role in nervous system development. The protein interacts with other cell surface molecules of the Ig super family and appears to be necessary for specific pathfinding by axonal growth cones during development (Lane et al., 1996). Lane et al. (1996) cloned the human homolog (NRCAM) of the chicken gene from a fetal brain library. Like its chicken counterpart, the predicted 1,275-amino acid protein has 6 V-like Ig domains and 5 fibronectin type III repeats. The transmembrane and intracellular domains of human and chicken NRCAM are entirely conserved and the proteins are 82% identical overall. Alternative splice variants were observed involving sequence around the fifth fibronectin repeat. Northern blots showed an approximately 7-kb transcript in all tissues of adult human brain examined.

Full details of the abovementioned studies are described in the following publications, the disclosure of which are hereby incorporated by reference:
Kayyem, J. F.; Roman, J. M.; de la Rosa, E. J.; Schwarz, U.; Dreyer, W. J.: Bravo/Nr-CAM is closely related to the cell adhesion molecules L1 and Ng-CAM and has a similar heterodimer structure. J. Cell. Biol. 118: 1259-1270, 1992.; and
Lane, R. P.; Chen, X.-N.; Yamakawa, K.; Vielmetter, J.; Korenberg, J. R.; Dreyer, W. J.: Characterization of a highly conserved human homolog to the chicken neural cell surface protein.

Further studies establishing the function and utilities of NRCAM are found in John Hopkins OMIM database record ID 601581, and in sited publications numbered 1508-1512 listed in the bibliography section hereinbelow, which are also hereby incorporated by reference. Chromosome 22 Open Reading Frame 23 (C22orf23, Accession NM_{—}032561) is another VGAM285 host target gene. C22orf23 BINDING SITE is HOST TARGET binding site found in the 3′ untranslated region of mRNA encoded by C22orf23, corresponding to a HOST TARGET binding site such as BINDING SITE I, BINDING SITE II or BINDING SITE III. Table 2 illustrates the complementarity of the nucleotide sequences of C22orf23 BINDING SITE, designated SEQ ID:2259, to the nucleotide sequence of VGAM285 RNA, herein designated VGAM RNA, also designated SEQ ID:620.

Another function of VGAM285 is therefore inhibition of Chromosome 22 Open Reading Frame 23 (C22orf23, Accession NM_{—}032561). Accordingly, utilities of VGAM285 include diagnosis, prevention and treatment of diseases and clinical conditions associated with C22orf23. SARM (Accession NM_{—}015077) is another VGAM285 host target gene. SARM BINDING SITE is HOST TARGET binding site found in the 3′ untranslated region of mRNA encoded by SARM, corresponding to a HOST TARGET binding site such as BINDING SITE I, BINDING SITE II or BINDING SITE III. Table 2 illustrates the complementarity of the nucleotide sequences of SARM BINDING SITE, designated SEQ ID: 1609, to the nucleotide sequence of VGAM285 RNA, herein designated VGAM RNA, also designated SEQ ID:620.

Another function of VGAM285 is therefore inhibition of SARM (Accession NM_{—}015077). Accordingly, utilities of VGAM285 include diagnosis, prevention and treatment of diseases and clinical conditions associated with SARM. LOC219918 (Accession XM_{—}166197) is another VGAM285 host target gene. LOC219918 BINDING SITE is HOST TARGET binding site found in the 3′ untranslated region of mRNA encoded by LOC219918, corresponding to a HOST TARGET binding site such as BINDING SITE I, BINDING SITE II or BINDING SITE III. Table 2 illustrates the complementarity of the nucleotide sequences of LOC219918 BINDING SITE, designated SEQ ID:3529, to the nucleotide sequence of VGAM285 RNA, herein designated VGAM RNA, also designated SEQ ID:620.

Another function of VGAM285 is therefore inhibition of LOC219918 (Accession XM_{—}166197). Accordingly, utilities of VGAM285 include diagnosis, prevention and treatment of diseases and clinical conditions associated with LOC219918. FIG. 1 further provides a conceptual description of a novel bioinformatically detected viral gene of the present invention, referred to here as Viral Genomic Address Messenger 286 (VGAM286) viral gene, which modulates expression of respective host target genes thereof, the function and utility of which host target genes is known in the art.

VGAM286 is a novel bioinformatically detected regulatory, non protein coding, viral micro RNA (miRNA) gene. The method by which VGAM286 was detected is described hereinabove with reference to FIGS. 1-8.

VGAM286 gene, herein designated VGAM GENE, is a viral gene contained in the genome of Vaccinia Virus. VGAM286 host target gene, herein designated VGAM HOST TARGET GENE, is a human gene contained in the human genome.

VGAM286 gene encodes a VGAM286 precursor RNA, herein designated VGAM PRECURSOR RNA. Similar to other miRNA genes, and unlike most ordinary genes, VGAM286 precursor RNA does not encode a protein. A nucleotide sequence identical or highly similar to the nucleotide sequence of VGAM286 precursor RNA is designated SEQ ID:272, and is provided hereinbelow with reference to the sequence listing part. Nucleotide sequence SEQ ID:272 is located at position 163795 relative to the genome of Vaccinia Virus.

VGAM286 precursor RNA folds onto itself, forming VGAM286 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM286 folded precursor RNA into VGAM286 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM286 RNA is designated SEQ ID:621, and is provided hereinbelow with reference to the sequence listing part.

VGAM286 host target gene, herein designated VGAM HOST TARGET GENE, encodes a corresponding messenger RNA, VGAM286 host target RNA, herein designated VGAM HOST TARGET RNA. VGAM286 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM286 RNA, herein designated VGAM RNA, binds complementarily to one or more host target binding sites located in untranslated regions of VGAM286 host target RNA, herein designated VGAM HOST TARGET RNA. This complementary binding is due to the fact that the nucleotide sequence of VGAM286 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM286 RNA, herein designated VGAM RNA, may have a different number of host target binding sites in untranslated regions of a VGAM286 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM286 RNA, herein designated VGAM RNA, to host target binding sites on VGAM286 host target RNA, herein designated VGAM HOST TARGET RNA, such as BINDING SITE I, BINDING SITE II and BINDING SITE III, inhibits translation of VGAM286 host target RNA into VGAM286 host target protein, herein designated VGAM HOST TARGET PROTEIN. VGAM host target protein is therefore outlined by a broken line.

It is appreciated that VGAM286 host target gene, herein designated VGAM HOST TARGET GENE, in fact represents a plurality of VGAM286 host target genes. The mRNA of each one of this plurality of VGAM286 host target genes comprises one or more host target binding sites, each having a nucleotide sequence which is at least partly complementary to VGAM286 RNA, herein designated VGAM RNA, and which when bound by VGAM286 RNA causes inhibition of translation of respective one or more VGAM286 host target proteins.

It is further appreciated by one skilled in the art that the mode of translational inhibition illustrated by FIG. 1 with specific reference to translational inhibition exerted by VGAM286 gene, herein designated VGAM GENE, on one or more VGAM286 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM286 is inhibition of expression of host target genes, as part of a novel viral mechanism of attacking a host. Accordingly, utilities of VGAM286 include diagnosis, prevention and treatment of viral infection by Vaccinia Virus. Specific functions, and accordingly utilities, of VGAM286 correlate with, and may be deduced from, the identity of the host target genes which VGAM286 binds and inhibits, and the function of these host target genes, as elaborated hereinbelow.

Nucleotide sequences of the VGAM286 precursor RNA, herein designated VGAM PRECURSOR RNA, and of the ‘diced’ VGAM286 RNA, herein designated VGAM RNA, and a schematic representation of the secondary folding of VGAM286 folded precursor RNA, herein designated VGAM FOLDED PRECURSOR RNA, of VGAM286 are further described hereinbelow with reference to Table 1.

Nucleotide sequences of host target binding sites, such as BINDING SITE-I, BINDING SITE-II and BINDING SITE-III of FIG. 1, found on VGAM286 host target RNA, and schematic representation of the complementarity of each of these host target binding sites to VGAM286 RNA, herein designated VGAM RNA, are described hereinbelow with reference to Table 2.

As mentioned hereinabove with reference to FIG. 1, a function of VGAM286 gene, herein designated VGAM is inhibition of expression of VGAM286 target genes. It is appreciated that specific functions, and accordingly utilities, of VGAM286 correlate with, and may be deduced from, the identity of the target genes which VGAM286 binds and inhibits, and the function of these target genes, as elaborated hereinbelow.

Fatty Acid Binding Protein 5 (psoriasis-associated) (FABP5, Accession NM_{—}001444) is a VGAM286 host target gene. FABP5 BINDING SITE is HOST TARGET binding site found in the 3′ untranslated region of mRNA encoded by FABP5, corresponding to a HOST TARGET binding site such as BINDING SITE I, BINDING SITE II or BINDING SITE III. Table 2 illustrates the complementarity of the nucleotide sequences of FABP5 BINDING SITE, designated SEQ ID:828, to the nucleotide sequence of VGAM286 RNA, herein designated VGAM RNA, also designated SEQ ID:621.

A function of VGAM286 is therefore inhibition of Fatty Acid Binding Protein 5 (psoriasis-associated) (FABP5, Accession NM_{—}001444), a gene which may have a role keratinocyte differentiation, highly expressed in psoriatic skin. Accordingly, utilities of VGAM286 include diagnosis, prevention and treatment of diseases and clinical conditions associated with FABP5. The function of FABP5 has been established by previous studies. Fatty acid-binding proteins (FABPs) bind free fatty acids and regulate lipid metabolism and transport. See also FABP3 (OMIM Ref. No. 134651). Using 2D gel electrophoresis, Madsen et al. (1992) isolated a fatty acid-binding protein, designated PAFABP for ‘psoriasis-associated fatty acid-binding protein,’ up-regulated in noncultured psoriatic skin keratinocytes. The protein was microsequenced, and degenerate oligonucleotides based on the amino acid sequence were used to screen a lambda-gt11 cDNA library prepared from noncultured psoriatic keratinocytes. The PAFABP cDNA encodes a 135-amino acid protein with molecular weight 15,164. By Northern blot analysis, the authors detected elevated levels of PAFABP message in noncultured psoriatic keratinocytes as compared to normal keratinocytes. PAFABP mRNA was also observed in transformed human epithelial cell lines, A431 epidermoid carcinoma cells, transformed human amnion (AMA) cells, and lymphoid Molt 4 cells. PAFABP protein was increased in primary normal keratinocytes induced to differentiate, as determined by 2D gel electrophoresis. Siegenthaler et al. (1994) characterized EFABP, which they determined to be identical to PAFABP by peptide sequencing. EFABP is abundant in psoriatic skin lesions. Purified EFABP specifically and reversibly binds oleic, linoleic, and stearic acids, but not cholesterol or retinoids. Use of antibodies specific for EFABP detected the protein in psoriatic lesions, heart, intestine, and adipose tissue. High levels of EFABP were also observed in abnormally differentiated epidermis. Immunohistochemistry localized EFABP to the stratum granulosum of normal skin.

Full details of the abovementioned studies are described in the following publications, the disclosure of which are hereby incorporated by reference:
Madsen, P.; Rasmussen, H. H.; Leffers, H.; Honore, B.; Celis, J. E.: Molecular cloning and expression of a novel keratinocyte protein (psoriasis-associated fatty acid-binding protein [PA-FABP]) that is highly up-regulated in psoriatic skin and that shares similarity to fatty acid-binding proteins. J. Invest. Derm. 99: 299-305, 1992.; and
Siegenthaler, G.; Hotz, R.; Chatellard-Gruaz, D.; Didierjean, L.; Hellman, U.; Saurat, J. H.: Purification and characterization of the human epidermal fatty acid-binding protein: local.

Further studies establishing the function and utilities of FABP5 are found in John Hopkins OMIM database record ID 605168, and in sited publications numbered 534-536 listed in the bibliography section hereinbelow, which are also hereby incorporated by reference. Zinc-fingers and Homeoboxes 1 (ZHX1, Accession NM_{—}007222) is another VGAM286 host target gene. ZHX1 BINDING SITE is HOST TARGET binding site found in the 3′ untranslated region of mRNA encoded by ZHX1, corresponding to a HOST TARGET binding site such as BINDING SITE I, BINDING SITE II or BINDING SITE III. Table 2 illustrates the complementarity of the nucleotide sequences of ZHX1 BINDING SITE, designated SEQ ID:1367, to the nucleotide sequence of VGAM286 RNA, herein designated VGAM RNA, also designated SEQ ID:621.

Another function of VGAM286 is therefore inhibition of Zinc-fingers and Homeoboxes 1 (ZHX1, Accession NM_{—}007222). Accordingly, utilities of VGAM286 include diagnosis, prevention and treatment of diseases and clinical conditions associated with ZHX1. KIAA0981 (Accession XM_{—}028867) is another VGAM286 host target gene. KIAA0981 BINDING SITE is HOST TARGET binding site found in the 3′ untranslated region of mRNA encoded by KIAA0981, corresponding to a HOST TARGET binding site such as BINDING SITE I, BINDING SITE II or BINDING SITE III. Table 2 illustrates the complementarity of the nucleotide sequences of KIAA0981 BINDING SITE, designated SEQ ID:2599, to the nucleotide sequence of VGAM286 RNA, herein designated VGAM RNA, also designated SEQ ID:621.

Another function of VGAM286 is therefore inhibition of KIAA0981 (Accession XM_{—}028867). Accordingly, utilities of VGAM286 include diagnosis, prevention and treatment of diseases and clinical conditions associated with KIAA0981. KIAA1615 (Accession XM_{—}044021) is another VGAM286 host target gene. KIAA1615 BINDING SITE is HOST TARGET binding site found in the 3′ untranslated region of mRNA encoded by KIAA1615, corresponding to a HOST TARGET binding site such as BINDING SITE I, BINDING SITE II or BINDING SITE III. Table 2 illustrates the complementarity of the nucleotide sequences of KIAA1615 BINDING SITE, designated SEQ ID:2829, to the nucleotide sequence of VGAM286 RNA, herein designated VGAM RNA, also designated SEQ ID:621.

Another function of VGAM286 is therefore inhibition of KIAA1615 (Accession XM_{—}044021). Accordingly, utilities of VGAM286 include diagnosis, prevention and treatment of diseases and clinical conditions associated with KIAA1615. SGP28 (Accession NM_{—}006061) is another VGAM286 host target gene. SGP28 BINDING SITE is HOST TARGET binding site found in the 3′ untranslated region of mRNA encoded by SGP28, corresponding to a HOST TARGET binding site such as BINDING SITE I, BINDING SITE II or BINDING SITE III. Table 2 illustrates the complementarity of the nucleotide sequences of SGP28 BINDING SITE, designated SEQ ID:1271, to the nucleotide sequence of VGAM286 RNA, herein designated VGAM RNA, also designated SEQ ID:621.

Another function of VGAM286 is therefore inhibition of SGP28 (Accession NM_{—}006061). Accordingly, utilities of VGAM286 include diagnosis, prevention and treatment of diseases and clinical conditions associated with SGP28. LOC201973 (Accession XM_{—}114414) is another VGAM286 host target gene. LOC201973 BINDING SITE is HOST TARGET binding site found in the 3′ untranslated region of mRNA encoded by LOC201973, corresponding to a HOST TARGET binding site such as BINDING SITE I, BINDING SITE II or BINDING SITE III. Table 2 illustrates the complementarity of the nucleotide sequences of LOC201973 BINDING SITE, designated SEQ ID:3456, to the nucleotide sequence of VGAM286 RNA, herein designated VGAM RNA, also designated SEQ ID:621.

Another function of VGAM286 is therefore inhibition of LOC201973 (Accession XM_{—}114414). Accordingly, utilities of VGAM286 include diagnosis, prevention and treatment of diseases and clinical conditions associated with LOC201973. FIG. 1 further provides a conceptual description of a novel bioinformatically detected viral gene of the present invention, referred to here as Viral Genomic Address Messenger 287 (VGAM287) viral gene, which modulates expression of respective host target genes thereof, the function and utility of which host target genes is known in the art.

VGAM287 is a novel bioinformatically detected regulatory, non protein coding, viral micro RNA (miRNA) gene. The method by which VGAM287 was detected is described hereinabove with reference to FIGS. 1-8.

VGAM287 gene, herein designated VGAM GENE, is a viral gene contained in the genome of Vaccinia Virus. VGAM287 host target gene, herein designated VGAM HOST TARGET GENE, is a human gene contained in the human genome.

VGAM287 gene encodes a VGAM287 precursor RNA, herein designated VGAM PRECURSOR RNA. Similar to other miRNA genes, and unlike most ordinary genes, VGAM287 precursor RNA does not encode a protein. A nucleotide sequence identical or highly similar to the nucleotide sequence of VGAM287 precursor RNA is designated SEQ ID:273, and is provided hereinbelow with reference to the sequence listing part. Nucleotide sequence SEQ ID:273 is located at position 163852 relative to the genome of Vaccinia Virus.

VGAM287 precursor RNA folds onto itself, forming VGAM287 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM287 folded precursor RNA into VGAM287 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM287 RNA is designated SEQ ID:622, and is provided hereinbelow with reference to the sequence listing part.

VGAM287 host target gene, herein designated VGAM HOST TARGET GENE, encodes a corresponding messenger RNA, VGAM287 host target RNA, herein designated VGAM HOST TARGET RNA. VGAM287 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM287 RNA, herein designated VGAM RNA, binds complementarily to one or more host target binding sites located in untranslated regions of VGAM287 host target RNA, herein designated VGAM HOST TARGET RNA. This complementary binding is due to the fact that the nucleotide sequence of VGAM287 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM287 RNA, herein designated VGAM RNA, may have a different number of host target binding sites in untranslated regions of a VGAM287 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM287 RNA, herein designated VGAM RNA, to host target binding sites on VGAM287 host target RNA, herein designated VGAM HOST TARGET RNA, such as BINDING SITE I, BINDING SITE II and BINDING SITE III, inhibits translation of VGAM287 host target RNA into VGAM287 host target protein, herein designated VGAM HOST TARGET PROTEIN. VGAM host target protein is therefore outlined by a broken line.

It is appreciated that VGAM287 host target gene, herein designated VGAM HOST TARGET GENE, in fact represents a plurality of VGAM287 host target genes. The mRNA of each one of this plurality of VGAM287 host target genes comprises one or more host target binding sites, each having a nucleotide sequence which is at least partly complementary to VGAM287 RNA, herein designated VGAM RNA, and which when bound by VGAM287 RNA causes inhibition of translation of respective one or more VGAM287 host target proteins.

It is further appreciated by one skilled in the art that the mode of translational inhibition illustrated by FIG. 1 with specific reference to translational inhibition exerted by VGAM287 gene, herein designated VGAM GENE, on one or more VGAM287 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM287 is inhibition of expression of host target genes, as part of a novel viral mechanism of attacking a host. Accordingly, utilities of VGAM287 include diagnosis, prevention and treatment of viral infection by Vaccinia Virus. Specific functions, and accordingly utilities, of VGAM287 correlate with, and may be deduced from, the identity of the host target genes which VGAM287 binds and inhibits, and the function of these host target genes, as elaborated hereinbelow.

Nucleotide sequences of the VGAM287 precursor RNA, herein designated VGAM PRECURSOR RNA, and of the ‘diced’ VGAM287 RNA, herein designated VGAM RNA, and a schematic representation of the secondary folding of VGAM287 folded precursor RNA, herein designated VGAM FOLDED PRECURSOR RNA, of VGAM287 are further described hereinbelow with reference to Table 1.

Nucleotide sequences of host target binding sites, such as BINDING SITE-I, BINDING SITE-II and BINDING SITE-III of FIG. 1, found on VGAM287 host target RNA, and schematic representation of the complementarity of each of these host target binding sites to VGAM287 RNA, herein designated VGAM RNA, are described hereinbelow with reference to Table 2.

As mentioned hereinabove with reference to FIG. 1, a function of VGAM287 gene, herein designated VGAM is inhibition of expression of VGAM287 target genes. It is appreciated that specific functions, and accordingly utilities, of VGAM287 correlate with, and may be deduced from, the identity of the target genes which VGAM287 binds and inhibits, and the function of these target genes, as elaborated hereinbelow.

LOC146237 (Accession XM_{—}096954) is a VGAM287 host target gene. LOC146237 BINDING SITE is HOST TARGET binding site found in the 3′ untranslated region of mRNA encoded by LOC146237, corresponding to a HOST TARGET binding site such as BINDING SITE I, BINDING SITE II or BINDING SITE III. Table 2 illustrates the complementarity of the nucleotide sequences of LOC146237 BINDING SITE, designated SEQ ID:3283, to the nucleotide sequence of VGAM287 RNA, herein designated VGAM RNA, also designated SEQ ID:622.

A function of VGAM287 is therefore inhibition of LOC146237 (Accession XM_{—}096954). Accordingly, utilities of VGAM287 include diagnosis, prevention and treatment of diseases and clinical conditions associated with LOC146237. LOC255146 (Accession XM_{—}170985) is another VGAM287 host target gene. LOC255146 BINDING SITE is HOST TARGET binding site found in the 5′ untranslated region of mRNA encoded by LOC255146, corresponding to a HOST TARGET binding site such as BINDING SITE I, BINDING SITE II or BINDING SITE III. Table 2 illustrates the complementarity of the nucleotide sequences of LOC255146 BINDING SITE, designated SEQ ID:3679, to the nucleotide sequence of VGAM287 RNA, herein designated VGAM RNA, also designated SEQ ID:622.

Another function of VGAM287 is therefore inhibition of LOC255146 (Accession XM_{—}170985). Accordingly, utilities of VGAM287 include diagnosis, prevention and treatment of diseases and clinical conditions associated with LOC255146. FIG. 1 further provides a conceptual description of a novel bioinformatically detected viral gene of the present invention, referred to here as Viral Genomic Address Messenger 288 (VGAM288) viral gene, which modulates expression of respective host target genes thereof, the function and utility of which host target genes is known in the art.

VGAM288 is a novel bioinformatically detected regulatory, non protein coding, viral micro RNA (miRNA) gene. The method by which VGAM288 was detected is described hereinabove with reference to FIGS. 1-8.

VGAM288 gene, herein designated VGAM GENE, is a viral gene contained in the genome of Vaccinia Virus. VGAM288 host target gene, herein designated VGAM HOST TARGET GENE, is a human gene contained in the human genome.

VGAM288 gene encodes a VGAM288 precursor RNA, herein designated VGAM PRECURSOR RNA. Similar to other miRNA genes, and unlike most ordinary genes, VGAM288 precursor RNA does not encode a protein. A nucleotide sequence identical or highly similar to the nucleotide sequence of VGAM288 precursor RNA is designated SEQ ID:274, and is provided hereinbelow with reference to the sequence listing part. Nucleotide sequence SEQ ID:274 is located at position 166608 relative to the genome of Vaccinia Virus.

VGAM288 precursor RNA folds onto itself, forming VGAM288 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM288 folded precursor RNA into VGAM288 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM288 RNA is designated SEQ ID:623, and is provided hereinbelow with reference to the sequence listing part.

VGAM288 host target gene, herein designated VGAM HOST TARGET GENE, encodes a corresponding messenger RNA, VGAM288 host target RNA, herein designated VGAM HOST TARGET RNA. VGAM288 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM288 RNA, herein designated VGAM RNA, binds complementarily to one or more host target binding sites located in untranslated regions of VGAM288 host target RNA, herein designated VGAM HOST TARGET RNA. This complementary binding is due to the fact that the nucleotide sequence of VGAM288 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM288 RNA, herein designated VGAM RNA, may have a different number of host target binding sites in untranslated regions of a VGAM288 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM288 RNA, herein designated VGAM RNA, to host target binding sites on VGAM288 host target RNA, herein designated VGAM HOST TARGET RNA, such as BINDING SITE I, BINDING SITE II and BINDING SITE III, inhibits translation of VGAM288 host target RNA into VGAM288 host target protein, herein designated VGAM HOST TARGET PROTEIN. VGAM host target protein is therefore outlined by a broken line.

It is appreciated that VGAM288 host target gene, herein designated VGAM HOST TARGET GENE, in fact represents a plurality of VGAM288 host target genes. The mRNA of each one of this plurality of VGAM288 host target genes comprises one or more host target binding sites, each having a nucleotide sequence which is at least partly complementary to VGAM288 RNA, herein designated VGAM RNA, and which when bound by VGAM288 RNA causes inhibition of translation of respective one or more VGAM288 host target proteins.

It is further appreciated by one skilled in the art that the mode of translational inhibition illustrated by FIG. 1 with specific reference to translational inhibition exerted by VGAM288 gene, herein designated VGAM GENE, on one or more VGAM288 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM288 is inhibition of expression of host target genes, as part of a novel viral mechanism of attacking a host. Accordingly, utilities of VGAM288 include diagnosis, prevention and treatment of viral infection by Vaccinia Virus. Specific functions, and accordingly utilities, of VGAM288 correlate with, and may be deduced from, the identity of the host target genes which VGAM288 binds and inhibits, and the function of these host target genes, as elaborated hereinbelow.

Nucleotide sequences of the VGAM288 precursor RNA, herein designated VGAM PRECURSOR RNA, and of the ‘diced’ VGAM288 RNA, herein designated VGAM RNA, and a schematic representation of the secondary folding of VGAM288 folded precursor RNA, herein designated VGAM FOLDED PRECURSOR RNA, of VGAM288 are further described hereinbelow with reference to Table 1.

Nucleotide sequences of host target binding sites, such as BINDING SITE-I, BINDING SITE-II and BINDING SITE-III of FIG. 1, found on VGAM288 host target RNA, and schematic representation of the complementarity of each of these host target binding sites to VGAM288 RNA, herein designated VGAM RNA, are described hereinbelow with reference to Table 2.

As mentioned hereinabove with reference to FIG. 1, a function of VGAM288 gene, herein designated VGAM is inhibition of expression of VGAM288 target genes. It is appreciated that specific functions, and accordingly utilities, of VGAM288 correlate with, and may be deduced from, the identity of the target genes which VGAM288 binds and inhibits, and the function of these target genes, as elaborated hereinbelow.

AT-binding Transcription Factor 1 (ATBF1, Accession NM_{—}006885) is a VGAM288 host target gene. ATBF1 BINDING SITE is HOST TARGET binding site found in the 5′ untranslated region of mRNA encoded by ATBF1, corresponding to a HOST TARGET binding site such as BINDING SITE I, BINDING SITE II or BINDING SITE III. Table 2 illustrates the complementarity of the nucleotide sequences of ATBF1 BINDING SITE, designated SEQ ID:1336, to the nucleotide sequence of VGAM288 RNA, herein designated VGAM RNA, also designated SEQ ID:623.

A function of VGAM288 is therefore inhibition of AT-binding Transcription Factor 1 (ATBF1, Accession NM_{—}006885). Accordingly, utilities of VGAM288 include diagnosis, prevention and treatment of diseases and clinical conditions associated with ATBF1. FIG. 1 further provides a conceptual description of a novel bioinformatically detected viral gene of the present invention, referred to here as Viral Genomic Address Messenger 289 (VGAM289) viral gene, which modulates expression of respective host target genes thereof, the function and utility of which host target genes is known in the art.

VGAM289 is a novel bioinformatically detected regulatory, non protein coding, viral micro RNA (miRNA) gene. The method by which VGAM289 was detected is described hereinabove with reference to FIGS. 1-8.

VGAM289 gene, herein designated VGAM GENE, is a viral gene contained in the genome of Vaccinia Virus. VGAM289 host target gene, herein designated VGAM HOST TARGET GENE, is a human gene contained in the human genome.

VGAM289 gene encodes a VGAM289 precursor RNA, herein designated VGAM PRECURSOR RNA. Similar to other miRNA genes, and unlike most ordinary genes, VGAM289 precursor RNA does not encode a protein. A nucleotide sequence identical or highly similar to the nucleotide sequence of VGAM289 precursor RNA is designated SEQ ID:275, and is provided hereinbelow with reference to the sequence listing part. Nucleotide sequence SEQ ID:275 is located at position 168231 relative to the genome of Vaccinia Virus.

VGAM289 precursor RNA folds onto itself, forming VGAM289 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM289 folded precursor RNA into VGAM289 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM289 RNA is designated SEQ ID:624, and is provided hereinbelow with reference to the sequence listing part.

VGAM289 host target gene, herein designated VGAM HOST TARGET GENE, encodes a corresponding messenger RNA, VGAM289 host target RNA, herein designated VGAM HOST TARGET RNA. VGAM289 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM289 RNA, herein designated VGAM RNA, binds complementarily to one or more host target binding sites located in untranslated regions of VGAM289 host target RNA, herein designated VGAM HOST TARGET RNA. This complementary binding is due to the fact that the nucleotide sequence of VGAM289 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM289 RNA, herein designated VGAM RNA, may have a different number of host target binding sites in untranslated regions of a VGAM289 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM289 RNA, herein designated VGAM RNA, to host target binding sites on VGAM289 host target RNA, herein designated VGAM HOST TARGET RNA, such as BINDING SITE I, BINDING SITE II and BINDING SITE III, inhibits translation of VGAM289 host target RNA into VGAM289 host target protein, herein designated VGAM HOST TARGET PROTEIN. VGAM host target protein is therefore outlined by a broken line.

It is appreciated that VGAM289 host target gene, herein designated VGAM HOST TARGET GENE, in fact represents a plurality of VGAM289 host target genes. The mRNA of each one of this plurality of VGAM289 host target genes comprises one or more host target binding sites, each having a nucleotide sequence which is at least partly complementary to VGAM289 RNA, herein designated VGAM RNA, and which when bound by VGAM289 RNA causes inhibition of translation of respective one or more VGAM289 host target proteins.

It is further appreciated by one skilled in the art that the mode of translational inhibition illustrated by FIG. 1 with specific reference to translational inhibition exerted by VGAM289 gene, herein designated VGAM GENE, on one or more VGAM289 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM289 is inhibition of expression of host target genes, as part of a novel viral mechanism of attacking a host. Accordingly, utilities of VGAM289 include diagnosis, prevention and treatment of viral infection by Vaccinia Virus. Specific functions, and accordingly utilities, of VGAM289 correlate with, and may be deduced from, the identity of the host target genes which VGAM289 binds and inhibits, and the function of these host target genes, as elaborated hereinbelow.

Nucleotide sequences of the VGAM289 precursor RNA, herein designated VGAM PRECURSOR RNA, and of the ‘diced’ VGAM289 RNA, herein designated VGAM RNA, and a schematic representation of the secondary folding of VGAM289 folded precursor RNA, herein designated VGAM FOLDED PRECURSOR RNA, of VGAM289 are further described hereinbelow with reference to Table 1.

Nucleotide sequences of host target binding sites, such as BINDING SITE-I, BINDING SITE-II and BINDING SITE-III of FIG. 1, found on VGAM289 host target RNA, and schematic representation of the complementarity of each of these host target binding sites to VGAM289 RNA, herein designated VGAM RNA, are described hereinbelow with reference to Table 2.

As mentioned hereinabove with reference to FIG. 1, a function of VGAM289 gene, herein designated VGAM is inhibition of expression of VGAM289 target genes. It is appreciated that specific functions, and accordingly utilities, of VGAM289 correlate with, and may be deduced from, the identity of the target genes which VGAM289 binds and inhibits, and the function of these target genes, as elaborated hereinbelow.

Fragile X Mental Retardation 2 (FMR2, Accession NM_{—}002025) is a VGAM289 host target gene. FMR2 BINDING SITE is HOST TARGET binding site found in the 3′ untranslated region of mRNA encoded by FMR2, corresponding to a HOST TARGET binding site such as BINDING SITE I, BINDING SITE II or BINDING SITE III. Table 2 illustrates the complementarity of the nucleotide sequences of FMR2 BINDING SITE, designated SEQ ID:889, to the nucleotide sequence of VGAM289 RNA, herein designated VGAM RNA, also designated SEQ ID:624.

A function of VGAM289 is therefore inhibition of Fragile X Mental Retardation 2 (FMR2, Accession NM_{—}002025). Accordingly, utilities of VGAM289 include diagnosis, prevention and treatment of diseases and clinical conditions associated with FMR2. Splicing Factor 1 (SF1, Accession NM_{—}004630) is another VGAM289 host target gene. SF1 BINDING SITE is HOST TARGET binding site found in the 3′ untranslated region of mRNA encoded by SF1, corresponding to a HOST TARGET binding site such as BINDING SITE I, BINDING SITE II or BINDING SITE III. Table 2 illustrates the complementarity of the nucleotide sequences of SF1 BINDING SITE, designated SEQ ID:1138, to the nucleotide sequence of VGAM289 RNA, herein designated VGAM RNA, also designated SEQ ID:624.

Another function of VGAM289 is therefore inhibition of Splicing Factor 1 (SF1, Accession NM_{—}004630), a gene which is a transcriptional repressor and splicing factor. Accordingly, utilities of VGAM289 include diagnosis, prevention and treatment of diseases and clinical conditions associated with SF1. The function of SF1 has been established by previous studies. Toda et al. (1994) isolated a gene, designated ZFM1 by them, from cosmids from the MEN1 (OMIM Ref. No. 131100) region of 11q13 using exon amplification. They then obtained cDNAs from cerebral, cerebellar, and fetal liver libraries. The predicted 623-amino acid protein contains a nuclear transport domain, a metal-binding or zinc finger motif, and glutamine- and proline-rich regions. It shows some sequence similarity to WT1 (OMIM Ref. No. 607102) and EGR2 (OMIM Ref. No. 129010). RT-PCR was used to show expression in the thyroid gland, pancreas, adrenal gland, and ovary. By differential screening of a cDNA library obtained from GMCSF (OMIM Ref. No. 138960)-stimulated human myeloid leukemia cells, Caslini et al. (1997) cloned 2 additional isoforms of the ZNF162 gene, designated B3 and B4, that encode 571- and 639-amino acid proteins, respectively. All of the ZNF162 isoforms contain a KH domain, a sequence motif present in proteins playing a major role in regulating cellular RNA metabolism.

Full details of the abovementioned studies are described in the following publications, the disclosure of which are hereby incorporated by reference:
Toda, T.; Iida, A.; Miwa, T.; Nakamura, Y.; Imai, T.: Isolation and characterization of a novel gene encoding nuclear protein at a locus (D11S636) tightly linked to multiple endocrine neoplasia type 1 (MEN1). Hum. Molec. Genet. 3: 465-470, 1994.; and
Caslini, C.; Spinelli, O.; Cazzaniga, G.; Golay, J.; De Gioia, L.; Pedretti, A.; Breviario, F.; Amaru, R.; Barbui, T.; Biondi, A.; Introna, M.; Rambaldi, A.: Identification of two novel is.

Further studies establishing the function and utilities of SF1 are found in John Hopkins OMIM database record ID 601516, and in sited publications numbered 643, 89 and 1550 listed in the bibliography section hereinbelow, which are also hereby incorporated by reference. BAL (Accession NM_{—}031458) is another VGAM289 host target gene. BAL BINDING SITE is HOST TARGET binding site found in the 3′ untranslated region of mRNA encoded by BAL, corresponding to a HOST TARGET binding site such as BINDING SITE I, BINDING SITE II or BINDING SITE III. Table 2 illustrates the complementarity of the nucleotide sequences of BAL BINDING SITE, designated SEQ ID:2201, to the nucleotide sequence of VGAM289 RNA, herein designated VGAM RNA, also designated SEQ ID:624.

Another function of VGAM289 is therefore inhibition of BAL (Accession NM_{—}031458). Accordingly, utilities of VGAM289 include diagnosis, prevention and treatment of diseases and clinical conditions associated with BAL. Butyrophilin, Sub family 3, Member A1 (BTN3A1, Accession NM_{—}007048) is another VGAM289 host target gene. BTN3A1 BINDING SITE is HOST TARGET binding site found in the 3′ untranslated region of mRNA encoded by BTN3A1, corresponding to a HOST TARGET binding site such as BINDING SITE I, BINDING SITE II or BINDING SITE III. Table 2 illustrates the complementarity of the nucleotide sequences of BTN3A1 BINDING SITE, designated SEQ ID:1355, to the nucleotide sequence of VGAM289 RNA, herein designated VGAM RNA, also designated SEQ ID:624.

Another function of VGAM289 is therefore inhibition of Butyrophilin, Sub family 3, Member A1 (BTN3A1, Accession NM_{—}007048). Accordingly, utilities of VGAM289 include diagnosis, prevention and treatment of diseases and clinical conditions associated with BTN3A1. Phosphodiesterase 4D Interacting Protein (myomegalin) (PDE4DIP, Accession NM_{—}014644) is another VGAM289 host target gene. PDE4DIP BINDING SITE is HOST TARGET binding site found in the 3′ untranslated region of mRNA encoded by PDE4DIP, corresponding to a HOST TARGET binding site such as BINDING SITE I, BINDING SITE II or BINDING SITE III. Table 2 illustrates the complementarity of the nucleotide sequences of PDE4DIP BINDING SITE, designated SEQ ID:1512, to the nucleotide sequence of VGAM289 RNA, herein designated VGAM RNA, also designated SEQ ID:624.

Another function of VGAM289 is therefore inhibition of Phosphodiesterase 4D Interacting Protein (myomegalin) (PDE4DIP, Accession NM_{—}014644). Accordingly, utilities of VGAM289 include diagnosis, prevention and treatment of diseases and clinical conditions associated with PDE4DIP. LOC200251 (Accession XM_{—}114173) is another VGAM289 host target gene. LOC200251 BINDING SITE is HOST TARGET binding site found in the 3′ untranslated region of mRNA encoded by LOC200251, corresponding to a HOST TARGET binding site such as BINDING SITE I, BINDING SITE II or BINDING SITE III. Table 2 illustrates the complementarity of the nucleotide sequences of LOC200251 BINDING SITE, designated SEQ ID:3447, to the nucleotide sequence of VGAM289 RNA, herein designated VGAM RNA, also designated SEQ ID:624.

Another function of VGAM289 is therefore inhibition of LOC200251 (Accession XM_{—}114173). Accordingly, utilities of VGAM289 include diagnosis, prevention and treatment of diseases and clinical conditions associated with LOC200251. FIG. 1 further provides a conceptual description of a novel bioinformatically detected viral gene of the present invention, referred to here as Viral Genomic Address Messenger 290 (VGAM290) viral gene, which modulates expression of respective host target genes thereof, the function and utility of which host target genes is known in the art.

VGAM290 is a novel bioinformatically detected regulatory, non protein coding, viral micro RNA (miRNA) gene. The method by which VGAM290 was detected is described hereinabove with reference to FIGS. 1-8.

VGAM290 gene, herein designated VGAM GENE, is a viral gene contained in the genome of Vaccinia Virus. VGAM290 host target gene, herein designated VGAM HOST TARGET GENE, is a human gene contained in the human genome.

VGAM290 gene encodes a VGAM290 precursor RNA, herein designated VGAM PRECURSOR RNA. Similar to other miRNA genes, and unlike most ordinary genes, VGAM290 precursor RNA does not encode a protein. A nucleotide sequence identical or highly similar to the nucleotide sequence of VGAM290 precursor RNA is designated SEQ ID:276, and is provided hereinbelow with reference to the sequence listing part. Nucleotide sequence SEQ ID:276 is located at position 166723 relative to the genome of Vaccinia Virus.

VGAM290 precursor RNA folds onto itself, forming VGAM290 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM290 folded precursor RNA into VGAM290 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM290 RNA is designated SEQ ID:625, and is provided hereinbelow with reference to the sequence listing part.

VGAM290 host target gene, herein designated VGAM HOST TARGET GENE, encodes a corresponding messenger RNA, VGAM290 host target RNA, herein designated VGAM HOST TARGET RNA. VGAM290 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM290 RNA, herein designated VGAM RNA, binds complementarily to one or more host target binding sites located in untranslated regions of VGAM290 host target RNA, herein designated VGAM HOST TARGET RNA. This complementary binding is due to the fact that the nucleotide sequence of VGAM290 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM290 RNA, herein designated VGAM RNA, may have a different number of host target binding sites in untranslated regions of a VGAM290 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM290 RNA, herein designated VGAM RNA, to host target binding sites on VGAM290 host target RNA, herein designated VGAM HOST TARGET RNA, such as BINDING SITE I, BINDING SITE II and BINDING SITE III, inhibits translation of VGAM290 host target RNA into VGAM290 host target protein, herein designated VGAM HOST TARGET PROTEIN. VGAM host target protein is therefore outlined by a broken line.

It is appreciated that VGAM290 host target gene, herein designated VGAM HOST TARGET GENE, in fact represents a plurality of VGAM290 host target genes. The mRNA of each one of this plurality of VGAM290 host target genes comprises one or more host target binding sites, each having a nucleotide sequence which is at least partly complementary to VGAM290 RNA, herein designated VGAM RNA, and which when bound by VGAM290 RNA causes inhibition of translation of respective one or more VGAM290 host target proteins.

It is further appreciated by one skilled in the art that the mode of translational inhibition illustrated by FIG. 1 with specific reference to translational inhibition exerted by VGAM290 gene, herein designated VGAM GENE, on one or more VGAM290 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM290 is inhibition of expression of host target genes, as part of a novel viral mechanism of attacking a host. Accordingly, utilities of VGAM290 include diagnosis, prevention and treatment of viral infection by Vaccinia Virus. Specific functions, and accordingly utilities, of VGAM290 correlate with, and may be deduced from, the identity of the host target genes which VGAM290 binds and inhibits, and the function of these host target genes, as elaborated hereinbelow.

Nucleotide sequences of the VGAM290 precursor RNA, herein designated VGAM PRECURSOR RNA, and of the ‘diced’ VGAM290 RNA, herein designated VGAM RNA, and a schematic representation of the secondary folding of VGAM290 folded precursor RNA, herein designated VGAM FOLDED PRECURSOR RNA, of VGAM290 are further described hereinbelow with reference to Table 1.

Nucleotide sequences of host target binding sites, such as BINDING SITE-I, BINDING SITE-II and BINDING SITE-III of FIG. 1, found on VGAM290 host target RNA, and schematic representation of the complementarity of each of these host target binding sites to VGAM290 RNA, herein designated VGAM RNA, are described hereinbelow with reference to Table 2.

As mentioned hereinabove with reference to FIG. 1, a function of VGAM290 gene, herein designated VGAM is inhibition of expression of VGAM290 target genes. It is appreciated that specific functions, and accordingly utilities, of VGAM290 correlate with, and may be deduced from, the identity of the target genes which VGAM290 binds and inhibits, and the function of these target genes, as elaborated hereinbelow.

Cyclin-dependent Kinase (CDC2-like) 10 (CDK10, Accession NM_{—}052988) is a VGAM290 host target gene. CDK10 BINDING SITE is HOST TARGET binding site found in the 3′ untranslated region of mRNA encoded by CDK10, corresponding to a HOST TARGET binding site such as BINDING SITE I, BINDING SITE II or BINDING SITE III. Table 2 illustrates the complementarity of the nucleotide sequences of CDK10 BINDING SITE, designated SEQ ID:2354, to the nucleotide sequence of VGAM290 RNA, herein designated VGAM RNA, also designated SEQ ID:625.

A function of VGAM290 is therefore inhibition of Cyclin-dependent Kinase (CDC2-like) 10 (CDK10, Accession NM_{—}052988), a gene which plays a pivotal role in the regulation of the eukaryotic cell cycle. Accordingly, utilities of VGAM290 include diagnosis, prevention and treatment of diseases and clinical conditions associated with CDK10. The function of CDK10 has been established by previous studies. Cyclin-dependent kinases (CDKs) are CDC2 (OMIM Ref. No. 116940)-related kinases that bind to cyclin to form active holoenzymes that play a pivotal role in the regulation of the eukaryotic cell cycle. To identify additional CDC2-like protein kinases, Brambilla and Draetta (1994) performed RT-PCR on human tumor cell line mRNA using degenerate oligonucleotides based on regions conserved among CDC2-related proteins. They used a resulting PCR product to screen a HeLa cell library and isolated a partial cDNA encoding a novel protein kinase. The 5-prime end of the cDNA was obtained using RACE. Brambilla and Draetta (1994) designated the predicted 360-amino acid protein PISSLRE, based on the amino acid sequence of the region corresponding to the conserved CDC2 PSTAIRE motif. PISSLRE contains all the structural elements characteristic of CDKs and unique extensions at both ends. Sequence comparisons revealed that it shares 41% and 50% protein sequence identity with CDC2 and CDC2L1 (OMIM Ref. No. 176873), respectively. By Northern blot analysis, the authors determined that PISSLRE was expressed broadly in human tissues as a 2-kb mRNA. An additional 3.5-kb transcript was observed in some tissues. Using a combination of library screening and 5-prime RACE, Grana et al. (1994) isolated PISSLRE cDNAs that differed significantly at both ends from those isolated by Brambilla and Draetta (1994). Brambilla and Draetta (1994) attributed the differences to alternative splicing. Grana et al. (1994) were unable to identify any ATG initiation codons upstream of the sequence encoding the catalytic domain of the putative kinase. They suggested that translation may initiate at 1 of 3 non-ATG initiation codons.

Full details of the abovementioned studies are described in the following publications, the disclosure of which are hereby incorporated by reference:
Bullrich, F.; MacLachlan, T. K.; Sang, N.; Druck, T.; Veronese, M. L.; Allen, S. L.; Chiorazzi, N.; Koff, A.; Heubner, K.; Croce, C. M.; Giordano, A.: Chromosomal mapping of members of the cdc2 family of protein kinases, cdk3, cdk6, PISSLRE, and PITALRE, and a cdk inhibitor, p27-Kip1, to regions involved in human cancer. Cancer Res. 55: 1199-1205, 1995.; and
Grana, X.; Claudio, P. P.; De Luca, A.; Sang, N.; Giordano, A.: PISSLRE, a human novel CDC2-related protein kinase. Oncogene 9: 2097-2103, 1994.

Further studies establishing the function and utilities of CDK10 are found in John Hopkins OMIM database record ID 603464, and in sited publications numbered 66 and 664 listed in the bibliography section hereinbelow, which are also hereby incorporated by reference. Collagen, Type I, Alpha 1 (COL1A1, Accession NM_{—}000088) is another VGAM290 host target gene. COL1A1 BINDING SITE is HOST TARGET binding site found in the 3′ untranslated region of mRNA encoded by COL1A1, corresponding to a HOST TARGET binding site such as BINDING SITE I, BINDING SITE II or BINDING SITE III. Table 2 illustrates the complementarity of the nucleotide sequences of COL1A1 BINDING SITE, designated SEQ ID:704, to the nucleotide sequence of VGAM290 RNA, herein designated VGAM RNA, also designated SEQ ID:625.

Another function of VGAM290 is therefore inhibition of Collagen, Type I, Alpha 1 (COL1A1, Accession NM_{—}000088). Accordingly, utilities of VGAM290 include diagnosis, prevention and treatment of diseases and clinical conditions associated with COL1A1. Espin (ESPN, Accession NM_{—}031475) is another VGAM290 host target gene. ESPN BINDING SITE is HOST TARGET binding site found in the 3′ untranslated region of mRNA encoded by ESPN, corresponding to a HOST TARGET binding site such as BINDING SITE I, BINDING SITE II or BINDING SITE III. Table 2 illustrates the complementarity of the nucleotide sequences of ESPN BINDING SITE, designated SEQ ID:2210, to the nucleotide sequence of VGAM290 RNA, herein designated VGAM RNA, also designated SEQ ID:625.

Another function of VGAM290 is therefore inhibition of Espin (ESPN, Accession NM_{—}031475), a gene which a membrane-cytoskeletal assemblages. Accordingly, utilities of VGAM290 include diagnosis, prevention and treatment of diseases and clinical conditions associated with ESPN. The function of ESPN has been established by previous studies. Ectoplasmic specializations are membrane-cytoskeletal assemblages found in Sertoli cells at sites of attachment to elongate spermatids or neighboring Sertoli cells. Bartles et al. (1996) identified the rat actin-bundling protein espin, which is localized to ectoplasmic specializations. The 836-amino acid espin protein had a molecular mass of approximately 110 kD in SDS gels. Northern blot analysis detected a 2.9-kb espin transcript only in rat testis; a minor 1.7-kb transcript was detected in small intestine and kidney. Bartles et al. (1998) identified a 30-kD, 253-amino acid isoform of rat espin that localized to brush border microvilli in the intestine and kidney. Espin and small espin share a 167-amino acid C-terminal peptide that includes a 116-amino acid C-terminal actin-bundling module that is necessary and sufficient for actin bundle formation in vitro; however, they contain different N termini. Bartles et al. (1998) and Chen et al. (1999) determined that unlike many actin-bundling proteins, the rat espins bind actin filaments with high affinity, and their actin-bundling activities are not inhibited by calcium. Zheng et al. (2000) determined that espins are present in hair cell stereocilia and uncovered a connection between the espin gene and jerker mouse, a recessive mutation that causes hair cell degeneration, deafness, and vestibular dysfunction. The tissues of jerker mice did not accumulate espin proteins but contained normal levels of espin mRNAs. The authors identified a frameshift mutation in the espin gene of jerker mice that affected the espin C-terminal actin-bundling module. These data suggested that jerker mice are espin null and that the jerker phenotype results from a mutation in the espin gene. Animal model experiments lend further support to the function of ESPN. Zheng et al. (2000) determined that espins are present in hair cell stereocilia and uncovered a connection between the espin gene and jerker mouse, a recessive mutation that causes hair cell degeneration, deafness, and vestibular dysfunction. The tissues of jerker mice did not accumulate espin proteins but contained normal levels of espin mRNAs. The authors identified a frameshift mutation in the espin gene of jerker mice that affected the espin C-terminal actin-bundling module. These data suggested that jerker mice are espin null and that the jerker phenotype results from a mutation in the espin gene. It is appreciated that the abovementioned animal model for ESPN is acknowledged by those skilled in the art as a scientifically valid animal model, as can be further appreciated from the publications sited hereinbelow.

Full details of the abovementioned studies are described in the following publications, the disclosure of which are hereby incorporated by reference:
Chen, B.; Li, A.; Wang, D.; Wang, M.; Zheng, L.; Bartles, J. R.: Espin contains an additional actin-binding site in its N terminus and is a major actin-bundling protein of the Sertoli cell-spermatid ectoplasmic specialization junctional plaque. Molec. Biol. Cell 10: 4327-4339, 1999.; and
Zheng, L.; Sekerkova, G.; Vranich, K.; Tilney, L. G.; Mugnaini, E.; Bartles, J. R.: The deaf jerker mouse has a mutation in the gene encoding the espin actin-bundling proteins of hair.

Further studies establishing the function and utilities of ESPN are found in John Hopkins OMIM database record ID 606351, and in sited publications numbered 1492-1495 listed in the bibliography section hereinbelow, which are also hereby incorporated by reference. Glutamate Receptor, Ionotropic, AMPA 1 (GRIA1, Accession NM_{—}000827) is another VGAM290 host target gene. GRIA1 BINDING SITE is HOST TARGET binding site found in the 3′ untranslated region of mRNA encoded by GRIA1, corresponding to a HOST TARGET binding site such as BINDING SITE I, BINDING SITE II or BINDING SITE III. Table 2 illustrates the complementarity of the nucleotide sequences of GRIA1 BINDING SITE, designated SEQ ID:779, to the nucleotide sequence of VGAM290 RNA, herein designated VGAM RNA, also designated SEQ ID:625.

Another function of VGAM290 is therefore inhibition of Glutamate Receptor, Ionotropic, AMPA 1 (GRIA1, Accession NM_{—}000827), a gene which acts as an excitatory neurotransmitter at many synapses in the central nervous system. Accordingly, utilities of VGAM290 include diagnosis, prevention and treatment of diseases and clinical conditions associated with GRIA1. The function of GRIA1 has been established by previous studies. To monitor changes in alpha-amino-3-hydroxy-5-methyl-4-isoxazole propionate (AMPA) receptor distribution in living neurons, Shi et al. (1999) tagged the AMPA receptor subunit GluR1 with green fluorescent protein (GFP). GluR1-GFP was functional and was transiently expressed in hippocampal CA1 neurons. In dendrites visualized with 2-photon laser scanning microscopy or electron microscopy, most of the GluR1-GFP was intracellular, mimicking endogenous GluR1 distribution. Tetanic synaptic stimulation induced a rapid delivery of tagged receptors into dendritic spines, as well as clusters in dendrite shafts. As they could be blocked by NMDA receptor antagonists, these postsynaptic trafficking events required synaptic NMDA receptor activation, and Shi et al. (1999) concluded that they may contribute to the enhanced AMPA receptor-mediated transmission observed during long-term potentiation and activity-dependent synaptic maturation. Animal model experiments lend further support to the function of GRIA1. Zamanillo et al. (1999) generated mice lacking the AMPA receptor subunit GluRA, also known as GluR1, by homologous recombination. Homozygous knockout mice exhibited normal development, life expectancy, and fine structure of neuronal dendrites and synapses. They were smaller than their littermates during the first postnatal weeks, but after weaning their size was normal. In hippocampal CA1 pyramidal neurons, GluRA −/− mice showed a reduction in functional AMPA receptors, with the remaining receptors preferentially targeted to synapses. Thus, the CA1 soma-patch currents were strongly reduced but glutamatergic synaptic currents were unaltered; evoked dendritic and spinous calcium currents, calcium-dependent gene activation, and hippocampal field potentials were as in wildtype. In adult GluRA −/− mice, associative long-term potentiation was absent in CA3 to CA1 synapses, but spatial learning in the water maze was not impaired. The results suggested to Zamanillo et al. (1999) that CA1 hippocampal long-term potentiation is controlled by the number or subunit composition of AMPA receptors and show a dichotomy between long-term potentiation in CA1 and acquisition of spatial memory.

It is appreciated that the abovementioned animal model for GRIA1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Shi, S.-H.; Hayashi, Y.; Petralla, R. S.; Zaman, S. H.; Wenthold, R. J.; Svoboda, K.; Malinow, R.: Rapid spine delivery and redistribution of AMPA receptors after synaptic NMDA receptor activation. Science 284: 1811-1816, 1999.; and
Zamanillo, D.; Sprengel, R.; Hvalby, O.; Jensen, V.; Burnashev, N.; Rozov, A.; Kaiser, K. M. M.; Koster, H. J.; Borchardt, T.; Worley, P.; Lubke, J.; Frotscher, M.; Kelly, P. H.; Sommer.

Further studies establishing the function and utilities of GRIA1 are found in John Hopkins OMIM database record ID 138248, and in sited publications numbered 2666-85 and 2667 listed in the bibliography section hereinbelow, which are also hereby incorporated by reference. Pleiomorphic Adenoma Gene 1 (PLAG1, Accession NM_{—}002655) is another VGAM290 host target gene. PLAG1 BINDING SITE is HOST TARGET binding site found in the 3′ untranslated region of mRNA encoded by PLAG1, corresponding to a HOST TARGET binding site such as BINDING SITE I, BINDING SITE II or BINDING SITE III. Table 2 illustrates the complementarity of the nucleotide sequences of PLAG1 BINDING SITE, designated SEQ ID:942, to the nucleotide sequence of VGAM290 RNA, herein designated VGAM RNA, also designated SEQ ID:625.

Another function of VGAM290 is therefore inhibition of Pleiomorphic Adenoma Gene 1 (PLAG1, Accession NM_{—}002655), a gene which contains a zinc finger domain. Accordingly, utilities of VGAM290 include diagnosis, prevention and treatment of diseases and clinical conditions associated with PLAG1. The function of PLAG1 and its association with various diseases and clinical conditions, has been established by previous studies, as described hereinabove with reference to VGAM50. SORCS1 (Accession NM_{—}052918) is another VGAM290 host target gene. SORCS1 BINDING SITE is HOST TARGET binding site found in the 3′ untranslated region of mRNA encoded by SORCS1, corresponding to a HOST TARGET binding site such as BINDING SITE I, BINDING SITE II or BINDING SITE III. Table 2 illustrates the complementarity of the nucleotide sequences of SORCS1 BINDING SITE, designated SEQ ID:2345, to the nucleotide sequence of VGAM290 RNA, herein designated VGAM RNA, also designated SEQ ID:625.

Another function of VGAM290 is therefore inhibition of SORCS1 (Accession NM_{—}052918). Accordingly, utilities of VGAM290 include diagnosis, prevention and treatment of diseases and clinical conditions associated with SORCS1. BM046 (Accession NM_{—}018460) is another VGAM290 host target gene. BM046 BINDING SITE is HOST TARGET binding site found in the 3′ untranslated region of mRNA encoded by BM046, corresponding to a HOST TARGET binding site such as BINDING SITE I, BINDING SITE II or BINDING SITE III. Table 2 illustrates the complementarity of the nucleotide sequences of BM046 BINDING SITE, designated SEQ ID: 1830, to the nucleotide sequence of VGAM290 RNA, herein designated VGAM RNA, also designated SEQ ID:625.

Another function of VGAM290 is therefore inhibition of BM046 (Accession NM_{—}018460). Accordingly, utilities of VGAM290 include diagnosis, prevention and treatment of diseases and clinical conditions associated with BM046. Kruppel-like Factor 12 (KLF12, Accession NM_{—}007249) is another VGAM290 host target gene. KLF12 BINDING SITE is HOST TARGET binding site found in the 3′ untranslated region of mRNA encoded by KLF12, corresponding to a HOST TARGET binding site such as BINDING SITE I, BINDING SITE II or BINDING SITE III. Table 2 illustrates the complementarity of the nucleotide sequences of KLF12 BINDING SITE, designated SEQ ID:1373, to the nucleotide sequence of VGAM290 RNA, herein designated VGAM RNA, also designated SEQ ID:625.

Another function of VGAM290 is therefore inhibition of Kruppel-like Factor 12 (KLF12, Accession NM_{—}007249). Accordingly, utilities of VGAM290 include diagnosis, prevention and treatment of diseases and clinical conditions associated with KLF12. Proteasome (prosome, macropain) Subunit, Alpha Type, 6 (PSMA6, Accession XM_{—}046642) is another VGAM290 host target gene. PSMA6 BINDING SITE is HOST TARGET binding site found in the 5′ untranslated region of mRNA encoded by PSMA6, corresponding to a HOST TARGET binding site such as BINDING SITE I, BINDING SITE II or BINDING SITE III. Table 2 illustrates the complementarity of the nucleotide sequences of PSMA6 BINDING SITE, designated SEQ ID:2880, to the nucleotide sequence of VGAM290 RNA, herein designated VGAM RNA, also designated SEQ ID:625.

Another function of VGAM290 is therefore inhibition of Proteasome (prosome, macropain) Subunit, Alpha Type, 6 (PSMA6, Accession XM_{—}046642). Accordingly, utilities of VGAM290 include diagnosis, prevention and treatment of diseases and clinical conditions associated with PSMA6. LOC157292 (Accession XM_{—}098740) is another VGAM290 host target gene. LOC157292 BINDING SITE is HOST TARGET binding site found in the 5′ untranslated region of mRNA encoded by LOC157292, corresponding to a HOST TARGET binding site such as BINDING SITE I, BINDING SITE II or BINDING SITE III. Table 2 illustrates the complementarity of the nucleotide sequences of LOC157292 BINDING SITE, designated SEQ ID:3377, to the nucleotide sequence of VGAM290 RNA, herein designated VGAM RNA, also designated SEQ ID:625.

Another function of VGAM290 is therefore inhibition of LOC157292 (Accession XM_{—}098740). Accordingly, utilities of VGAM290 include diagnosis, prevention and treatment of diseases and clinical conditions associated with LOC157292. FIG. 1 further provides a conceptual description of a novel bioinformatically detected viral gene of the present invention, referred to here as Viral Genomic Address Messenger 291 (VGAM291) viral gene, which modulates expression of respective host target genes thereof, the function and utility of which host target genes is known in the art.

VGAM291 is a novel bioinformatically detected regulatory, non protein coding, viral micro RNA (miRNA) gene. The method by which VGAM291 was detected is described hereinabove with reference to FIGS. 1-8.

VGAM291 gene, herein designated VGAM GENE, is a viral gene contained in the genome of Vaccinia Virus. VGAM291 host target gene, herein designated VGAM HOST TARGET GENE, is a human gene contained in the human genome.

VGAM291 gene encodes a VGAM291 precursor RNA, herein designated VGAM PRECURSOR RNA. Similar to other miRNA genes, and unlike most ordinary genes, VGAM291 precursor RNA does not encode a protein. A nucleotide sequence identical or highly similar to the nucleotide sequence of VGAM291 precursor RNA is designated SEQ ID:277, and is provided hereinbelow with reference to the sequence listing part. Nucleotide sequence SEQ ID:277 is located at position 165333 relative to the genome of Vaccinia Virus.

VGAM291 precursor RNA folds onto itself, forming VGAM291 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM291 folded precursor RNA into VGAM291 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM291 RNA is designated SEQ ID:626, and is provided hereinbelow with reference to the sequence listing part.

VGAM291 host target gene, herein designated VGAM HOST TARGET GENE, encodes a corresponding messenger RNA, VGAM291 host target RNA, herein designated VGAM HOST TARGET RNA. VGAM291 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM291 RNA, herein designated VGAM RNA, binds complementarily to one or more host target binding sites located in untranslated regions of VGAM291 host target RNA, herein designated VGAM HOST TARGET RNA. This complementary binding is due to the fact that the nucleotide sequence of VGAM291 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM291 RNA, herein designated VGAM RNA, may have a different number of host target binding sites in untranslated regions of a VGAM291 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM291 RNA, herein designated VGAM RNA, to host target binding sites on VGAM291 host target RNA, herein designated VGAM HOST TARGET RNA, such as BINDING SITE I, BINDING SITE II and BINDING SITE III, inhibits translation of VGAM291 host target RNA into VGAM291 host target protein, herein designated VGAM HOST TARGET PROTEIN. VGAM host target protein is therefore outlined by a broken line.

It is appreciated that VGAM291 host target gene, herein designated VGAM HOST TARGET GENE, in fact represents a plurality of VGAM291 host target genes. The mRNA of each one of this plurality of VGAM291 host target genes comprises one or more host target binding sites, each having a nucleotide sequence which is at least partly complementary to VGAM291 RNA, herein designated VGAM RNA, and which when bound by VGAM291 RNA causes inhibition of translation of respective one or more VGAM291 host target proteins.

It is further appreciated by one skilled in the art that the mode of translational inhibition illustrated by FIG. 1 with specific reference to translational inhibition exerted by VGAM291 gene, herein designated VGAM GENE, on one or more VGAM291 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM291 is inhibition of expression of host target genes, as part of a novel viral mechanism of attacking a host. Accordingly, utilities of VGAM291 include diagnosis, prevention and treatment of viral infection by Vaccinia Virus. Specific functions, and accordingly utilities, of VGAM291 correlate with, and may be deduced from, the identity of the host target genes which VGAM291 binds and inhibits, and the function of these host target genes, as elaborated hereinbelow.

Nucleotide sequences of the VGAM291 precursor RNA, herein designated VGAM PRECURSOR RNA, and of the ‘diced’ VGAM291 RNA, herein designated VGAM RNA, and a schematic representation of the secondary folding of VGAM291 folded precursor RNA, herein designated VGAM FOLDED PRECURSOR RNA, of VGAM291 are further described hereinbelow with reference to Table 1.

Nucleotide sequences of host target binding sites, such as BINDING SITE-I, BINDING SITE-II and BINDING SITE-III of FIG. 1, found on VGAM291 host target RNA, and schematic representation of the complementarity of each of these host target binding sites to VGAM291 RNA, herein designated VGAM RNA, are described hereinbelow with reference to Table 2.

As mentioned hereinabove with reference to FIG. 1, a function of VGAM291 gene, herein designated VGAM is inhibition of expression of VGAM291 target genes. It is appreciated that specific functions, and accordingly utilities, of VGAM291 correlate with, and may be deduced from, the identity of the target genes which VGAM291 binds and inhibits, and the function of these target genes, as elaborated hereinbelow.

Cadherin, EGF LAG Seven-pass G-type Receptor 3 (flamingo homolog, Drosophila) (CELSR3, Accession NM_{—}001407) is a VGAM291 host target gene. CELSR3 BINDING SITE is HOST TARGET binding site found in the 3′ untranslated region of mRNA encoded by CELSR3, corresponding to a HOST TARGET binding site such as BINDING SITE I, BINDING SITE II or BINDING SITE III. Table 2 illustrates the complementarity of the nucleotide sequences of CELSR3 BINDING SITE, designated SEQ ID:825, to the nucleotide sequence of VGAM291 RNA, herein designated VGAM RNA, also designated SEQ ID:626.

A function of VGAM291 is therefore inhibition of Cadherin, EGF LAG Seven-pass G-type Receptor 3 (flamingo homolog, Drosophila) (CELSR3, Accession NM_{—}001407), a gene which interacts in a homophilic manner in connecting cells. Accordingly, utilities of VGAM291 include diagnosis, prevention and treatment of diseases and clinical conditions associated with CELSR3. The function of CELSR3 and its association with various diseases and clinical conditions, has been established by previous studies, as described hereinabove with reference to VGAM162. Cell Matrix Adhesion Regulator (CMAR, Accession NM_{—}005200) is another VGAM291 host target gene. CMAR BINDING SITE is HOST TARGET binding site found in the 5′ untranslated region of mRNA encoded by CMAR, corresponding to a HOST TARGET binding site such as BINDING SITE I, BINDING SITE II or BINDING SITE III. Table 2 illustrates the complementarity of the nucleotide sequences of CMAR BINDING SITE, designated SEQ ID:1195, to the nucleotide sequence of VGAM291 RNA, herein designated VGAM RNA, also designated SEQ ID:626.

Another function of VGAM291 is therefore inhibition of Cell Matrix Adhesion Regulator (CMAR, Accession NM_{—}005200). Accordingly, utilities of VGAM291 include diagnosis, prevention and treatment of diseases and clinical conditions associated with CMAR. Fc Fragment of IgE, Low Affinity II, Receptor For (CD23A) (FCER2, Accession NM_{—}002002) is another VGAM291 host target gene. FCER2 BINDING SITE1 through FCER2 BINDING SITE3 are HOST TARGET binding sites found in untranslated regions of mRNA encoded by FCER2, corresponding to HOST TARGET binding sites such as BINDING SITE I, BINDING SITE II or BINDING SITE III. Table 2 illustrates the complementarity of the nucleotide sequences of FCER2 BINDING SITE1 through FCER2 BINDING SITE3, designated SEQ ID:881, SEQ ID:882 and SEQ ID:883 respectively, to the nucleotide sequence of VGAM291 RNA, herein designated VGAM RNA, also designated SEQ ID:626.

Another function of VGAM291 is therefore inhibition of Fc Fragment of IgE, Low Affinity II, Receptor For (CD23A) (FCER2, Accession NM_{—}002002), a gene which regulates the synthesis of IgE; contains an inverse RGD motif that binds to IgE. Accordingly, utilities of VGAM291 include diagnosis, prevention and treatment of diseases and clinical conditions associated with FCER2. The function of FCER2 has been established by previous studies. Ludin et al. (1987) cloned the cDNA coding for a human lymphocyte IgE receptor. Low-affinity receptors and secretor factors, which bind to immunoglobulins of the IgE isotype, play a key role in the regulation of human IgE synthesis. The nucleotide sequence of the cDNA predicted a polypeptide with 321 amino acids and a molecular weight of 36,281 daltons. The role of the IgE receptor in allergic diseases will be of interest. Furthermore, the receptor may be involved in determining the basic level of IgE in serum (OMIM Ref. No. 147050). The human leukocyte differentiation antigen CD23 (FCE2) is a key molecule for B-cell activation and growth. It is the low-affinity receptor for IgE. Furthermore, the truncated molecule can be secreted, then functioning as a potent mitogenic growth factor. Wendel-Hansen et al. (1990) showed by Southern analysis of DNAs from human/rodent cell hybrids that the CD23 gene is located on human chromosome 19. As part of a mapping of multiple probes to specific bands on chromosome 19 by fluorescence in situ hybridization, Trask et al. (1993) assigned the FCE2 gene to 19p13.3. Delespesse et al. (1989) studied the influence of IL4 and interferon on the production of CD23.

Full details of the abovementioned studies are described in the following publications, the disclosure of which are hereby incorporated by reference:
Ludin, C.; Hofstetter, H.; Sarfati, M.; Levy, C. A.; Suter, U.; Alaimo, D.; Kilchherr, E.; Frost, H.; Delespesse, G.: Cloning and expression of the cDNA coding for a human lymphocyte IgE receptor. EMBO J. 6: 109-114, 1987.; and
Wendel-Hansen, V.; Riviere, M.; Uno, M.; Jansson, I.; Szpirer, J.; Islam, M. Q.; Levan, G.; Klein, G.; Yodoi, J.; Rosen, A.; Szpirer, C.: The gene encoding CD23 leukocyte antigen (FCE2).

Further studies establishing the function and utilities of FCER2 are found in John Hopkins OMIM database record ID 151445, and in sited publications numbered 78-7 and 1058 listed in the bibliography section hereinbelow, which are also hereby incorporated by reference. NCSTN (Accession XM_{—}057331) is another VGAM291 host target gene. NCSTN BINDING SITE is HOST TARGET binding site found in the 3′ untranslated region of mRNA encoded by NCSTN, corresponding to a HOST TARGET binding site such as BINDING SITE I, BINDING SITE II or BINDING SITE III. Table 2 illustrates the complementarity of the nucleotide sequences of NCSTN BINDING SITE, designated SEQ ID:2979, to the nucleotide sequence of VGAM291 RNA, herein designated VGAM RNA, also designated SEQ ID:626.

Another function of VGAM291 is therefore inhibition of NCSTN (Accession XM_{—}057331), a gene which has a central role in presenilin-mediated processing of beta-amyloid precursor protein (beta-APP, MIM 104760) and some aspects of notch (MIM 190198)/glp-1 signaling in vivo. Accordingly, utilities of VGAM291 include diagnosis, prevention and treatment of diseases and clinical conditions associated with NCSTN. The function of NCSTN has been established by previous studies. Nicastrin is a type 1 transmembrane glycoprotein that interacts with both presenilin-1 (PS1; OMIM Ref. No. 104311) and presenilin-2 (PS2; 600759) and has a central role in presenilin-mediated processing of beta-amyloid precursor protein (beta-APP, 104760) and some aspects of notch (OMIM Ref. No. 190198)/glp-1 signaling in vivo. Nicastrin regulates gamma-secretase cleavage of the amyloid precursor protein by forming complexes with presenilins, in which most mutations causing familial early-onset Alzheimer disease (EOAD) have been found. The nicastrin gene maps to 1q23, a region that shows evidence for linkage to (Kehoe et al., 1999) and association with (Hiltunen et al., 2001) late-onset Alzheimer disease (LOAD). Dermaut et al. (2002) evaluated the contribution of genetic variations in NCSTN in 2 large series of patients with EOAD (onset at or before age 65 years) and LOAD (onset after age 65 years). In 78 familial EOAD cases, they found 14 NCSTN single-nucleotide polymorphisms (SNPs): 10 intronic SNPs, 3 silent mutations, and 1 missense mutation (N417Y). N417Y was thought not to be pathogenic, since it did not alter amyloid-beta secretion in an in vitro assay and its frequency was similar in case and control subjects. However, SNP haplotype estimation in 2 population-based series of Dutch patients with EOAD (n=116) and LOAD (n=240) indicated that the frequency of 1 SNP haplotype (designated HapB) was higher in the group with familial EOAD (7%), compared with the LOAD group (3%) and control group (3%). In patients with familial EOAD without the APOE epsilon-4 allele (107741.0016), the HapB frequency further increased, to 14%, resulting in a 4-fold increased risk (odds ratio=4.1). These results were considered compatible with an important role of gamma-secretase dysfunction in the etiology of familial EOAD.

Full details of the abovementioned studies are described in the following publications, the disclosure of which are hereby incorporated by reference:
Hiltunen, M.; Mannermaa, A.; Thompson, D.; Easton, D.; Pirskanen, M.; Helisalmi, S.; Koivisto, A. M.; Lehtovirta, M.; Ryynanen, M.; Soininen, H.: Genome-wide linkage disequilibrium mapping of late-onset Alzheimer's disease in Finland. Neurology 57: 1663-1668, 2001.; and
Kehoe, P.; Wavrant-De Vrieze, F.; Crook, R.; Wu, W. S.; Holmans, P.; Fenton, I.; Spurlock, G.; Norton, N.; Williams, H.; Williams, N.; Lovestone, S.; Perez-Tur, J.; Hutton, J.; and 10.

Further studies establishing the function and utilities of NCSTN are found in John Hopkins OMIM database record ID 605254, and in sited publications numbered 976-983 listed in the bibliography section hereinbelow, which are also hereby incorporated by reference. RNA Binding Motif Protein 10 (RBM10, Accession NM_{—}005676) is another VGAM291 host target gene. RBM10 BINDING SITE is HOST TARGET binding site found in the 3′ untranslated region of mRNA encoded by RBM10, corresponding to a HOST TARGET binding site such as BINDING SITE I, BINDING SITE II or BINDING SITE III. Table 2 illustrates the complementarity of the nucleotide sequences of RBM10 BINDING SITE, designated SEQ ID:1235, to the nucleotide sequence of VGAM291 RNA, herein designated VGAM RNA, also designated SEQ ID:626.

Another function of VGAM291 is therefore inhibition of RNA Binding Motif Protein 10 (RBM10, Accession NM_{—}005676), a gene which has RNA-binding activity. Accordingly, utilities of VGAM291 include diagnosis, prevention and treatment of diseases and clinical conditions associated with RBM10. The function of RBM10 has been established by previous studies. Nagase et al. (1995) cloned an RNA-binding protein as part of a random sequencing project of cDNAs from a myeloid cell line. They determined that this clone, which they termed KIAA0122, encodes a putative polypeptide of 880 amino acids that has significant homology with several RNA-binding proteins. They determined by Northern blotting that it is widely, perhaps ubiquitously, expressed. The S1 proteins are a group of nuclear proteins which occur in association with hnRNA in the cell nucleus. Inoue et al. (1996) cloned a rat homolog of human KIAA0122, which they termed S1-1. Based on protein microsequences, they noted that the rat S1-1 sequence had 2 ribonucleoprotein (RNP) motifs but was distinct from other S1 proteins. They showed that the S1-1 protein has RNA-binding activity.

Full details of the abovementioned studies are described in the following publications, the disclosure of which are hereby incorporated by reference:
Inoue, A.; Takahashi, K. P.; Kimura, M.; Watanabe, T.; Morisawa, S.: Molecular cloning of a RNA binding protein, S1-1. Nucleic Acids Res. 24: 2990-2997, 1996.; and
Nagase, T.; Seki, N.; Tanaka, A.; Ishikawa, K.; Nomura, N.: Prediction of the coding sequences of unidentified human genes. IV. The coding sequences of 40 new genes (KIAA0121-KIAA0160).

Further studies establishing the function and utilities of RBM10 are found in John Hopkins OMIM database record ID 300080, and in sited publications numbered 2522-2524 listed in the bibliography section hereinbelow, which are also hereby incorporated by reference. Tumor Necrosis Factor (ligand) Super family, Member 8 (TNFSF8, Accession NM_{—}001244) is another VGAM291 host target gene. TNFSF8 BINDING SITE is HOST TARGET binding site found in the 3′ untranslated region of mRNA encoded by TNFSF8, corresponding to a HOST TARGET binding site such as BINDING SITE I, BINDING SITE II or BINDING SITE III. Table 2 illustrates the complementarity of the nucleotide sequences of TNFSF8 BINDING SITE, designated SEQ ID:812, to the nucleotide sequence of VGAM291 RNA, herein designated VGAM RNA, also designated SEQ ID:626.

Another function of VGAM291 is therefore inhibition of Tumor Necrosis Factor (ligand) Super family, Member 8 (TNFSF8, Accession NM_{—}001244), a gene which cytokine that binds to tnfrsf8/cd30. induces proliferation of t cells. Accordingly, utilities of VGAM291 include diagnosis, prevention and treatment of diseases and clinical conditions associated with TNFSF8. The function of TNFSF8 and its association with various diseases and clinical conditions, has been established by previous studies, as described hereinabove with reference to VGAM165. V-yes-1 Yamaguchi Sarcoma Viral Oncogene Homolog 1 (YES1, Accession NM_{—}005433) is another VGAM291 host target gene. YES1 BINDING SITE is HOST TARGET binding site found in the 3′ untranslated region of mRNA encoded by YES1, corresponding to a HOST TARGET binding site such as BINDING SITE I, BINDING SITE II or BINDING SITE III. Table 2 illustrates the complementarity of the nucleotide sequences of YES1 BINDING SITE, designated SEQ ID:1210, to the nucleotide sequence of VGAM291 RNA, herein designated VGAM RNA, also designated SEQ ID:626.

Another function of VGAM291 is therefore inhibition of V-yes-1 Yamaguchi Sarcoma Viral Oncogene Homolog 1 (YES1, Accession NM_{—}005433), a gene which is a putative protein-tyrosine kinase. Accordingly, utilities of VGAM291 include diagnosis, prevention and treatment of diseases and clinical conditions associated with YES1. The function of YES1 and its association with various diseases and clinical conditions, has been established by previous studies, as described hereinabove with reference to VGAM256. Zinc Finger Protein 256 (ZNF256, Accession NM_{—}005773) is another VGAM291 host target gene. ZNF256 BINDING SITE is HOST TARGET binding site found in the 5′ untranslated region of mRNA encoded by ZNF256, corresponding to a HOST TARGET binding site such as BINDING SITE I, BINDING SITE II or BINDING SITE III. Table 2 illustrates the complementarity of the nucleotide sequences of ZNF256 BINDING SITE, designated SEQ ID:1247, to the nucleotide sequence of VGAM291 RNA, herein designated VGAM RNA, also designated SEQ ID:626.

Another function of VGAM291 is therefore inhibition of Zinc Finger Protein 256 (ZNF256, Accession NM_{—}005773). Accordingly, utilities of VGAM291 include diagnosis, prevention and treatment of diseases and clinical conditions associated with ZNF256. HLA-B Associated Transcript 8 (BAT8, Accession NM_{—}006709) is another VGAM291 host target gene. BAT8 BINDING SITE is HOST TARGET binding site found in the 3′ untranslated region of mRNA encoded by BAT8, corresponding to a HOST TARGET binding site such as BINDING SITE I, BINDING SITE II or BINDING SITE III. Table 2 illustrates the complementarity of the nucleotide sequences of BAT8 BINDING SITE, designated SEQ ID:1325, to the nucleotide sequence of VGAM291 RNA, herein designated VGAM RNA, also designated SEQ ID:626.

Another function of VGAM291 is therefore inhibition of HLA-B Associated Transcript 8 (BAT8, Accession NM_{—}006709). Accordingly, utilities of VGAM291 include diagnosis, prevention and treatment of diseases and clinical conditions associated with BAT8. Complexin 1 (CPLX1, Accession NM_{—}006651) is another VGAM291 host target gene. CPLX1 BINDING SITE is HOST TARGET binding site found in the 3′ untranslated region of mRNA encoded by CPLX1, corresponding to a HOST TARGET binding site such as BINDING SITE I, BINDING SITE II or BINDING SITE III. Table 2 illustrates the complementarity of the nucleotide sequences of CPLX1 BINDING SITE, designated SEQ ID:1318, to the nucleotide sequence of VGAM291 RNA, herein designated VGAM RNA, also designated SEQ ID:626.

Another function of VGAM291 is therefore inhibition of Complexin 1 (CPLX1, Accession NM_{—}006651). Accordingly, utilities of VGAM291 include diagnosis, prevention and treatment of diseases and clinical conditions associated with CPLX1. DKFZP566K0524 (Accession XM_{—}045128) is another VGAM291 host target gene. DKFZP566K0524 BINDING SITE is HOST TARGET binding site found in the 3′ untranslated region of mRNA encoded by DKFZP566K0524, corresponding to a HOST TARGET binding site such as BINDING SITE I, BINDING SITE II or BINDING SITE III. Table 2 illustrates the complementarity of the nucleotide sequences of DKFZP566K0524 BINDING SITE, designated SEQ ID:2855, to the nucleotide sequence of VGAM291 RNA, herein designated VGAM RNA, also designated SEQ ID:626.

Another function of VGAM291 is therefore inhibition of DKFZP566K0524 (Accession XM_{—}045128). Accordingly, utilities of VGAM291 include diagnosis, prevention and treatment of diseases and clinical conditions associated with DKFZP566K0524. FLJ12891 (Accession NM_{—}024950) is another VGAM291 host target gene. FLJ12891 BINDING SITE is HOST TARGET binding site found in the 3′ untranslated region of mRNA encoded by FLJ12891, corresponding to a HOST TARGET binding site such as BINDING SITE I, BINDING SITE II or BINDING SITE III. Table 2 illustrates the complementarity of the nucleotide sequences of FLJ12891 BINDING SITE, designated SEQ ID:2117, to the nucleotide sequence of VGAM291 RNA, herein designated VGAM RNA, also designated SEQ ID:626.

Another function of VGAM291 is therefore inhibition of FLJ12891 (Accession NM_{—}024950). Accordingly, utilities of VGAM291 include diagnosis, prevention and treatment of diseases and clinical conditions associated with FLJ12891. GR6 (Accession NM_{—}007354) is another VGAM291 host target gene. GR6 BINDING SITE is HOST TARGET binding site found in the 3′ untranslated region of mRNA encoded by GR6, corresponding to a HOST TARGET binding site such as BINDING SITE I, BINDING SITE II or BINDING SITE III. Table 2 illustrates the complementarity of the nucleotide sequences of GR6 BINDING SITE, designated SEQ ID: 1396, to the nucleotide sequence of VGAM291 RNA, herein designated VGAM RNA, also designated SEQ ID:626.

Another function of VGAM291 is therefore inhibition of GR6 (Accession NM_{—}007354). Accordingly, utilities of VGAM291 include diagnosis, prevention and treatment of diseases and clinical conditions associated with GR6. KIAA0455 (Accession XM_{—}051785) is another VGAM291 host target gene. KIAA0455 BINDING SITE is HOST TARGET binding site found in the 3′ untranslated region of mRNA encoded by KIAA0455, corresponding to a HOST TARGET binding site such as BINDING SITE I, BINDING SITE II or BINDING SITE III. Table 2 illustrates the complementarity of the nucleotide sequences of KIAA0455 BINDING SITE, designated SEQ ID:2947, to the nucleotide sequence of VGAM291 RNA, herein designated VGAM RNA, also designated SEQ ID:626.

Another function of VGAM291 is therefore inhibition of KIAA0455 (Accession XM_{—}051785). Accordingly, utilities of VGAM291 include diagnosis, prevention and treatment of diseases and clinical conditions associated with KIAA0455. KIAA0513 (Accession NM_{—}014732) is another VGAM291 host target gene. KIAA0513 BINDING SITE is HOST TARGET binding site found in the 3′ untranslated region of mRNA encoded by KIAA0513, corresponding to a HOST TARGET binding site such as BINDING SITE I, BINDING SITE II or BINDING SITE III. Table 2 illustrates the complementarity of the nucleotide sequences of KIAA0513 BINDING SITE, designated SEQ ID:1536, to the nucleotide sequence of VGAM291 RNA, herein designated VGAM RNA, also designated SEQ ID:626.

Another function of VGAM291 is therefore inhibition of KIAA0513 (Accession NM_{—}014732). Accordingly, utilities of VGAM291 include diagnosis, prevention and treatment of diseases and clinical conditions associated with KIAA0513. KIAA1655 (Accession XM_{—}039442) is another VGAM291 host target gene. KIAA1655 BINDING SITE is HOST TARGET binding site found in the 5′ untranslated region of mRNA encoded by KIAA1655, corresponding to a HOST TARGET binding site such as BINDING SITE I, BINDING SITE II or BINDING SITE III. Table 2 illustrates the complementarity of the nucleotide sequences of KIAA1655 BINDING SITE, designated SEQ ID:2753, to the nucleotide sequence of VGAM291 RNA, herein designated VGAM RNA, also designated SEQ ID:626.

Another function of VGAM291 is therefore inhibition of KIAA1655 (Accession XM_{—}039442). Accordingly, utilities of VGAM291 include diagnosis, prevention and treatment of diseases and clinical conditions associated with KIAA1655. SCAN Domain Containing 2 (SCAND2, Accession NM_{—}033634) is another VGAM291 host target gene. SCAND2 BINDING SITE is HOST TARGET binding site found in the 3′ untranslated region of mRNA encoded by SCAND2, corresponding to a HOST TARGET binding site such as BINDING SITE I, BINDING SITE II or BINDING SITE III. Table 2 illustrates the complementarity of the nucleotide sequences of SCAND2 BINDING SITE, designated SEQ ID:2338, to the nucleotide sequence of VGAM291 RNA, herein designated VGAM RNA, also designated SEQ ID:626.

Another function of VGAM291 is therefore inhibition of SCAN Domain Containing 2 (SCAND2, Accession NM_{—}033634). Accordingly, utilities of VGAM291 include diagnosis, prevention and treatment of diseases and clinical conditions associated with SCAND2. WD Repeat Domain 5B (WDR5B, Accession NM_{—}019069) is another VGAM291 host target gene. WDR5B BINDING SITE is HOST TARGET binding site found in the 3′ untranslated region of mRNA encoded by WDR5B, corresponding to a HOST TARGET binding site such as BINDING SITE I, BINDING SITE II or BINDING SITE III. Table 2 illustrates the complementarity of the nucleotide sequences of WDR5B BINDING SITE, designated SEQ ID:1878, to the nucleotide sequence of VGAM291 RNA, herein designated VGAM RNA, also designated SEQ ID:626.

Another function of VGAM291 is therefore inhibition of WD Repeat Domain 5B (WDR5B, Accession NM_{—}019069). Accordingly, utilities of VGAM291 include diagnosis, prevention and treatment of diseases and clinical conditions associated with WDR5B. LOC146330 (Accession XM_{—}085426) is another VGAM291 host target gene. LOC146330 BINDING SITE is HOST TARGET binding site found in the 5′ untranslated region of mRNA encoded by LOC146330, corresponding to a HOST TARGET binding site such as BINDING SITE I, BINDING SITE II or BINDING SITE III. Table 2 illustrates the complementarity of the nucleotide sequences of LOC146330 BINDING SITE, designated SEQ ID:3088, to the nucleotide sequence of VGAM291 RNA, herein designated VGAM RNA, also designated SEQ ID:626.

Another function of VGAM291 is therefore inhibition of LOC146330 (Accession XM_{—}085426). Accordingly, utilities of VGAM291 include diagnosis, prevention and treatment of diseases and clinical conditions associated with LOC146330. LOC148181 (Accession XM_{—}086083) is another VGAM291 host target gene. LOC148181 BINDING SITE is HOST TARGET binding site found in the 5′ untranslated region of mRNA encoded by LOC148181, corresponding to a HOST TARGET binding site such as BINDING SITE I, BINDING SITE II or BINDING SITE III. Table 2 illustrates the complementarity of the nucleotide sequences of LOC148181 BINDING SITE, designated SEQ ID:3117, to the nucleotide sequence of VGAM291 RNA, herein designated VGAM RNA, also designated SEQ ID:626.

Another function of VGAM291 is therefore inhibition of LOC148181 (Accession XM_{—}086083). Accordingly, utilities of VGAM291 include diagnosis, prevention and treatment of diseases and clinical conditions associated with LOC148181. LOC150271 (Accession XM_{—}097859) is another VGAM291 host target gene. LOC150271 BINDING SITE is HOST TARGET binding site found in the 5′ untranslated region of mRNA encoded by LOC150271, corresponding to a HOST TARGET binding site such as BINDING SITE I, BINDING SITE II or BINDING SITE III. Table 2 illustrates the complementarity of the nucleotide sequences of LOC150271 BINDING SITE, designated SEQ ID:3326, to the nucleotide sequence of VGAM291 RNA, herein designated VGAM RNA, also designated SEQ ID:626.

Another function of VGAM291 is therefore inhibition of LOC150271 (Accession XM_{—}097859). Accordingly, utilities of VGAM291 include diagnosis, prevention and treatment of diseases and clinical conditions associated with LOC150271. LOC150605 (Accession XM_{—}097927) is another VGAM291 host target gene. LOC150605 BINDING SITE is HOST TARGET binding site found in the 5′ untranslated region of mRNA encoded by LOC150605, corresponding to a HOST TARGET binding site such as BINDING SITE I, BINDING SITE II or BINDING SITE III. Table 2 illustrates the complementarity of the nucleotide sequences of LOC150605 BINDING SITE, designated SEQ ID:3331, to the nucleotide sequence of VGAM291 RNA, herein designated VGAM RNA, also designated SEQ ID:626.

Another function of VGAM291 is therefore inhibition of LOC150605 (Accession XM_{—}097927). Accordingly, utilities of VGAM291 include diagnosis, prevention and treatment of diseases and clinical conditions associated with LOC150605. LOC153277 (Accession XM_{—}098346) is another VGAM291 host target gene. LOC153277 BINDING SITE is HOST TARGET binding site found in the 3′ untranslated region of mRNA encoded by LOC153277, corresponding to a HOST TARGET binding site such as BINDING SITE I, BINDING SITE II or BINDING SITE III. Table 2 illustrates the complementarity of the nucleotide sequences of LOC153277 BINDING SITE, designated SEQ ID:3368, to the nucleotide sequence of VGAM291 RNA, herein designated VGAM RNA, also designated SEQ ID:626.

Another function of VGAM291 is therefore inhibition of LOC153277 (Accession XM_{—}098346). Accordingly, utilities of VGAM291 include diagnosis, prevention and treatment of diseases and clinical conditions associated with LOC153277. LOC154990 (Accession XM_{—}088109) is another VGAM291 host target gene. LOC154990 BINDING SITE is HOST TARGET binding site found in the 5′ untranslated region of mRNA encoded by LOC154990, corresponding to a HOST TARGET binding site such as BINDING SITE I, BINDING SITE II or BINDING SITE III. Table 2 illustrates the complementarity of the nucleotide sequences of LOC154990 BINDING SITE, designated SEQ ID:3196, to the nucleotide sequence of VGAM291 RNA, herein designated VGAM RNA, also designated SEQ ID:626.

Another function of VGAM291 is therefore inhibition of LOC154990 (Accession XM_{—}088109). Accordingly, utilities of VGAM291 include diagnosis, prevention and treatment of diseases and clinical conditions associated with LOC154990. LOC158014 (Accession XM_{—}088442) is another VGAM291 host target gene. LOC158014 BINDING SITE is HOST TARGET binding site found in the 5′ untranslated region of mRNA encoded by LOC158014, corresponding to a HOST TARGET binding site such as BINDING SITE I, BINDING SITE II or BINDING SITE III. Table 2 illustrates the complementarity of the nucleotide sequences of LOC158014 BINDING SITE, designated SEQ ID:3209, to the nucleotide sequence of VGAM291 RNA, herein designated VGAM RNA, also designated SEQ ID:626.

Another function of VGAM291 is therefore inhibition of LOC158014 (Accession XM_{—}088442). Accordingly, utilities of VGAM291 include diagnosis, prevention and treatment of diseases and clinical conditions associated with LOC158014. LOC158056 (Accession XM_{—}088463) is another VGAM291 host target gene. LOC158056 BINDING SITE is HOST TARGET binding site found in the 5′ untranslated region of mRNA encoded by LOC158056, corresponding to a HOST TARGET binding site such as BINDING SITE I, BINDING SITE II or BINDING SITE III. Table 2 illustrates the complementarity of the nucleotide sequences of LOC158056 BINDING SITE, designated SEQ ID:3210, to the nucleotide sequence of VGAM291 RNA, herein designated VGAM RNA, also designated SEQ ID:626.

Another function of VGAM291 is therefore inhibition of LOC158056 (Accession XM_{—}088463). Accordingly, utilities of VGAM291 include diagnosis, prevention and treatment of diseases and clinical conditions associated with LOC158056. LOC202908 (Accession XM_{—}114602) is another VGAM291 host target gene. LOC202908 BINDING SITE is HOST TARGET binding site found in the 3′ untranslated region of mRNA encoded by LOC202908, corresponding to a HOST TARGET binding site such as BINDING SITE I, BINDING SITE II or BINDING SITE III. Table 2 illustrates the complementarity of the nucleotide sequences of LOC202908 BINDING SITE, designated SEQ ID:3462, to the nucleotide sequence of VGAM291 RNA, herein designated VGAM RNA, also designated SEQ ID:626.

Another function of VGAM291 is therefore inhibition of LOC202908 (Accession XM_{—}114602). Accordingly, utilities of VGAM291 include diagnosis, prevention and treatment of diseases and clinical conditions associated with LOC202908. LOC220143 (Accession XM_{—}168046) is another VGAM291 host target gene. LOC220143 BINDING SITE1 and LOC220143 BINDING SITE2 are HOST TARGET binding sites found in untranslated regions of mRNA encoded by LOC220143, corresponding to HOST TARGET binding sites such as BINDING SITE I, BINDING SITE II or BINDING SITE III. Table 2 illustrates the complementarity of the nucleotide sequences of LOC220143 BINDING SITE1 and LOC220143 BINDING SITE2, designated SEQ ID:3610 and SEQ ID:3611 respectively, to the nucleotide sequence of VGAM291 RNA, herein designated VGAM RNA, also designated SEQ ID:626.

Another function of VGAM291 is therefore inhibition of LOC220143 (Accession XM_{—}168046). Accordingly, utilities of VGAM291 include diagnosis, prevention and treatment of diseases and clinical conditions associated with LOC220143. LOC255096 (Accession XM_{—}174913) is another VGAM291 host target gene. LOC255096 BINDING SITE is HOST TARGET binding site found in the 3′ untranslated region of mRNA encoded by LOC255096, corresponding to a HOST TARGET binding site such as BINDING SITE I, BINDING SITE II or BINDING SITE III. Table 2 illustrates the complementarity of the nucleotide sequences of LOC255096 BINDING SITE, designated SEQ ID:3744, to the nucleotide sequence of VGAM291 RNA, herein designated VGAM RNA, also designated SEQ ID:626.

Another function of VGAM291 is therefore inhibition of LOC255096 (Accession XM_{—}174913). Accordingly, utilities of VGAM291 include diagnosis, prevention and treatment of diseases and clinical conditions associated with LOC255096. LOC255975 (Accession XM_{—}171083) is another VGAM291 host target gene. LOC255975 BINDING SITE is HOST TARGET binding site found in the 5′ untranslated region of mRNA encoded by LOC255975, corresponding to a HOST TARGET binding site such as BINDING SITE I, BINDING SITE II or BINDING SITE III. Table 2 illustrates the complementarity of the nucleotide sequences of LOC255975 BINDING SITE, designated SEQ ID:3686, to the nucleotide sequence of VGAM291 RNA, herein designated VGAM RNA, also designated SEQ ID:626.

Another function of VGAM291 is therefore inhibition of LOC255975 (Accession XM_{—}171083). Accordingly, utilities of VGAM291 include diagnosis, prevention and treatment of diseases and clinical conditions associated with LOC255975. LOC256878 (Accession XM_{—}173042) is another VGAM291 host target gene. LOC256878 BINDING SITE is HOST TARGET binding site found in the 3′ untranslated region of mRNA encoded by LOC256878, corresponding to a HOST TARGET binding site such as BINDING SITE I, BINDING SITE II or BINDING SITE III. Table 2 illustrates the complementarity of the nucleotide sequences of LOC256878 BINDING SITE, designated SEQ ID:3720, to the nucleotide sequence of VGAM291 RNA, herein designated VGAM RNA, also designated SEQ ID:626.

Another function of VGAM291 is therefore inhibition of LOC256878 (Accession XM_{—}173042). Accordingly, utilities of VGAM291 include diagnosis, prevention and treatment of diseases and clinical conditions associated with LOC256878. LOC51333 (Accession NM_{—}016643) is another VGAM291 host target gene. LOC51333 BINDING SITE is HOST TARGET binding site found in the 3′ untranslated region of mRNA encoded by LOC51333, corresponding to a HOST TARGET binding site such as BINDING SITE I, BINDING SITE II or BINDING SITE III. Table 2 illustrates the complementarity of the nucleotide sequences of LOC51333 BINDING SITE, designated SEQ ID:1705, to the nucleotide sequence of VGAM291 RNA, herein designated VGAM RNA, also designated SEQ ID:626.

Another function of VGAM291 is therefore inhibition of LOC51333 (Accession NM_{—}016643). Accordingly, utilities of VGAM291 include diagnosis, prevention and treatment of diseases and clinical conditions associated with LOC51333. LOC89919 (Accession XM_{—}027244) is another VGAM291 host target gene. LOC89919 BINDING SITE is HOST TARGET binding site found in the 3′ untranslated region of mRNA encoded by LOC89919, corresponding to a HOST TARGET binding site such as BINDING SITE I, BINDING SITE II or BINDING SITE III. Table 2 illustrates the complementarity of the nucleotide sequences of LOC89919 BINDING SITE, designated SEQ ID:2580, to the nucleotide sequence of VGAM291 RNA, herein designated VGAM RNA, also designated SEQ ID:626.

Another function of VGAM291 is therefore inhibition of LOC89919 (Accession XM_{—}027244). Accordingly, utilities of VGAM291 include diagnosis, prevention and treatment of diseases and clinical conditions associated with LOC89919. FIG. 1 further provides a conceptual description of a novel bioinformatically detected viral gene of the present invention, referred to here as Viral Genomic Address Messenger 292 (VGAM292) viral gene, which modulates expression of respective host target genes thereof, the function and utility of which host target genes is known in the art.

VGAM292 is a novel bioinformatically detected regulatory, non protein coding, viral micro RNA (miRNA) gene. The method by which VGAM292 was detected is described hereinabove with reference to FIGS. 1-8.

VGAM292 gene, herein designated VGAM GENE, is a viral gene contained in the genome of Vaccinia Virus. VGAM292 host target gene, herein designated VGAM HOST TARGET GENE, is a human gene contained in the human genome.

VGAM292 gene encodes a VGAM292 precursor RNA, herein designated VGAM PRECURSOR RNA. Similar to other miRNA genes, and unlike most ordinary genes, VGAM292 precursor RNA does not encode a protein. A nucleotide sequence identical or highly similar to the nucleotide sequence of VGAM292 precursor RNA is designated SEQ ID:278, and is provided hereinbelow with reference to the sequence listing part. Nucleotide sequence SEQ ID:278 is located at position 165010 relative to the genome of Vaccinia Virus.

VGAM292 precursor RNA folds onto itself, forming VGAM292 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM292 folded precursor RNA into VGAM292 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM292 RNA is designated SEQ ID:627, and is provided hereinbelow with reference to the sequence listing part.

VGAM292 host target gene, herein designated VGAM HOST TARGET GENE, encodes a corresponding messenger RNA, VGAM292 host target RNA, herein designated VGAM HOST TARGET RNA. VGAM292 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM292 RNA, herein designated VGAM RNA, binds complementarily to one or more host target binding sites located in untranslated regions of VGAM292 host target RNA, herein designated VGAM HOST TARGET RNA. This complementary binding is due to the fact that the nucleotide sequence of VGAM292 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM292 RNA, herein designated VGAM RNA, may have a different number of host target binding sites in untranslated regions of a VGAM292 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM292 RNA, herein designated VGAM RNA, to host target binding sites on VGAM292 host target RNA, herein designated VGAM HOST TARGET RNA, such as BINDING SITE I, BINDING SITE II and BINDING SITE III, inhibits translation of VGAM292 host target RNA into VGAM292 host target protein, herein designated VGAM HOST TARGET PROTEIN. VGAM host target protein is therefore outlined by a broken line.

It is appreciated that VGAM292 host target gene, herein designated VGAM HOST TARGET GENE, in fact represents a plurality of VGAM292 host target genes. The mRNA of each one of this plurality of VGAM292 host target genes comprises one or more host target binding sites, each having a nucleotide sequence which is at least partly complementary to VGAM292 RNA, herein designated VGAM RNA, and which when bound by VGAM292 RNA causes inhibition of translation of respective one or more VGAM292 host target proteins.

It is further appreciated by one skilled in the art that the mode of translational inhibition illustrated by FIG. 1 with specific reference to translational inhibition exerted by VGAM292 gene, herein designated VGAM GENE, on one or more VGAM292 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM292 is inhibition of expression of host target genes, as part of a novel viral mechanism of attacking a host. Accordingly, utilities of VGAM292 include diagnosis, prevention and treatment of viral infection by Vaccinia Virus. Specific functions, and accordingly utilities, of VGAM292 correlate with, and may be deduced from, the identity of the host target genes which VGAM292 binds and inhibits, and the function of these host target genes, as elaborated hereinbelow.

Nucleotide sequences of the VGAM292 precursor RNA, herein designated VGAM PRECURSOR RNA, and of the ‘diced’ VGAM292 RNA, herein designated VGAM RNA, and a schematic representation of the secondary folding of VGAM292 folded precursor RNA, herein designated VGAM FOLDED PRECURSOR RNA, of VGAM292 are further described hereinbelow with reference to Table 1.

Nucleotide sequences of host target binding sites, such as BINDING SITE-I, BINDING SITE-II and BINDING SITE-III of FIG. 1, found on VGAM292 host target RNA, and schematic representation of the complementarity of each of these host target binding sites to VGAM292 RNA, herein designated VGAM RNA, are described hereinbelow with reference to Table 2.

As mentioned hereinabove with reference to FIG. 1, a function of VGAM292 gene, herein designated VGAM is inhibition of expression of VGAM292 target genes. It is appreciated that specific functions, and accordingly utilities, of VGAM292 correlate with, and may be deduced from, the identity of the target genes which VGAM292 binds and inhibits, and the function of these target genes, as elaborated hereinbelow.

Interleukin 2 Receptor, Alpha (IL2RA, Accession NM_{—}000417) is a VGAM292 host target gene. IL2RA BINDING SITE is HOST TARGET binding site found in the 3′ untranslated region of mRNA encoded by IL2RA, corresponding to a HOST TARGET binding site such as BINDING SITE I, BINDING SITE II or BINDING SITE III. Table 2 illustrates the complementarity of the nucleotide sequences of IL2RA BINDING SITE, designated SEQ ID:739, to the nucleotide sequence of VGAM292 RNA, herein designated VGAM RNA, also designated SEQ ID:627.

A function of VGAM292 is therefore inhibition of Interleukin 2 Receptor, Alpha (IL2RA, Accession NM_{—}000417), a gene which plays a role in T cell mediated immune response. Accordingly, utilities of VGAM292 include diagnosis, prevention and treatment of diseases and clinical conditions associated with IL2RA. The function of IL2RA has been established by previous studies. The action of the T-cell growth factor interleukin-2 (IL2; 147680) requires the presence of a cell surface receptor. As most peripheral as well as thymic T cells do not carry the receptor in vivo, the regulated expression of IL2R appears to be a safeguard against a catastrophic spread of T-cell proliferation by an immunogenic stimulus. The receptor is a heterodimer, consisting of 1 alpha and 1 beta chain; the beta chain (OMIM Ref. No. 146710) was not characterized until 1989. The receptor molecule, a glycoprotein, has a relative mass of about 55,000. Its intracellular precursor is smaller. Leonard et al. (1983) used a monoclonal antibody for T-cell growth factor to characterize the receptor. Yang et al. (2001) analyzed T-cell subsets and levels of cytokine IL2 and soluble IL2 receptor in the peripheral blood of patients with normal pressure glaucoma (NPG; 606657) and primary open angle glaucoma (POAG; 137760) and compared them to values in age-matched controls. They found an increased frequency of CD8+/HLA-DR+ lymphocytes in patients with NPG and increased CD3+/CD8+ lymphocytes in both NPG and POAG patients. CD5+ lymphocytes were higher only in POAG patients. The mean concentration of soluble IL2R was higher in NPG and POAG patients than in controls although the IL2 concentration was similar in patients and controls. The authors concluded that the immune system might play an important role in initiation or progression of glaucomatous optic neuropathy in some patients.

Full details of the abovementioned studies are described in the following publications, the disclosure of which are hereby incorporated by reference:
Leonard, W. J.; Donlon, T. A.; Lebo, R. V.; Greene, W. C.: Localization of the gene encoding the human interleukin-2 receptor on chromosome 10. Science 228: 1547-1549, 1985.; and
Yang, J.; Patil, R. V.; Yu, H.; Gordon, M.; Wax, M. B.: T cell subsets and sIL-2R/IL-2 levels in patients with glaucoma. Am. J. Ophthal. 131: 421-426, 2001.

Further studies establishing the function and utilities of IL2RA are found in John Hopkins OMIM database record ID 147730, and in sited publications numbered 2562-2570, 2792, 2583-2584, 142, 2585-259 and 143 listed in the bibliography section hereinbelow, which are also hereby incorporated by reference. Inositol Polyphosphate-5-phosphatase, 145 kDa (INPP5D, Accession XM_{—}096169) is another VGAM292 host target gene. INPP5D BINDING SITE is HOST TARGET binding site found in the 3′ untranslated region of mRNA encoded by INPP5D, corresponding to a HOST TARGET binding site such as BINDING SITE I, BINDING SITE II or BINDING SITE III. Table 2 illustrates the complementarity of the nucleotide sequences of INPP5D BINDING SITE, designated SEQ ID:3263, to the nucleotide sequence of VGAM292 RNA, herein designated VGAM RNA, also designated SEQ ID:627.

Another function of VGAM292 is therefore inhibition of Inositol Polyphosphate-5-phosphatase, 145 kDa (INPP5D, Accession XM_{—}096169), a gene which hydrolyzes Ins(1,3,4,5)P4 and PtdIns(3,4,5)P3; contains an SH2-domain. Accordingly, utilities of VGAM292 include diagnosis, prevention and treatment of diseases and clinical conditions associated with INPP5D. The function of INPP5D has been established by previous studies. The phosphatidylinositols serve as precursors for a number of different messenger molecules. Agonist stimulation of cells results in phosphatidylinositol turnover and the generation of inositol 1,4,5-triphosphate (Ins(1,4,5)P3), which mobilizes intracellular calcium. The inositol-polyphosphate 5-phosphatase (INPP5) enzymes hydrolyze Ins(1,4,5)P3 in a signal-terminating reaction. Known INPP5s include the 40-kD INPP5A (OMIM Ref. No. 600106), the 75-kD INPP5B (OMIM Ref. No. 147264), and the enzyme associated with Lowe oculocerebrorenal syndrome (OMIM Ref. No. 309000). Damen et al. (1996) cloned and sequenced a cDNA encoding a 145-kD protein from a mouse hematopoietic cell line; the protein became tyrosine phosphorylated and associated with SHC (OMIM Ref. No. 600560) after cytokine stimulation. Based on its domains and enzymatic activity, Damen et al. (1996) named this protein SHIP for ‘SH2-containing inositol phosphatase Liu et al. (1998) studied the expression of the Ship gene during mouse development. They found that the gene is expressed in late primitive-streak stage embryos (7.5 days post coitum), when hematopoiesis is thought to begin, and the expression is restricted to the hematopoietic lineage. In adult mice, Ship expression continues in most cells of hematopoietic origin, including granulocytes, monocytes, and lymphocytes, and is also found in the spermatids of the testis. Furthermore, the level of Ship expression is developmentally regulated during T-cell maturation. These results suggested a possible role for Ship in the differentiation and maintenance of the hematopoietic lineages and in spermatogenesis Animal model experiments lend further support to the function of INPP5D. Because Ship −/− mice contain increased numbers of osteoclast precursors, i.e., macrophages, Takeshita et al. (2002) examined bones from these animals and found that osteoclast number was increased 2-fold. The increased number was the result of prolonged lifespan of these cells and hypersensitivity of precursors to macrophage colony-stimulating factor (MCSF; 120420) and receptor activator of nuclear factor-kappa-B ligand (RANKL; 602642). Similar to the osteoclasts of Paget disease of bone (OMIM Ref. No. 602080), Ship −/− osteoclasts were enlarged, containing upwards of 100 nuclei, and exhibited enhanced resorptive activity. Moreover, as in Paget disease, serum levels of interleukin-6 (OMIM Ref. No. 147620) were markedly increased in Ship −/− mice. Consistent with accelerated resorptive activity, a 22% loss of bone-mineral density and a 49% decrease in fracture energy were observed. Thus, SHIP negatively regulates osteoclast formation and function, and the absence of this enzyme results in severe osteoporosis

It is appreciated that the abovementioned animal model for INPP5D is acknowledged by those skilled in the art as a scientifically valid animal model, as can be further appreciated from the publications sited hereinbelow.

Full details of the abovementioned studies are described in the following publications, the disclosure of which are hereby incorporated by reference:
Liu, Q.; Shalaby, F.; Jones, J.; Bouchard, D.; Dumont, D. J.: The SH2-containing inositol polyphosphate 5-phosphatase, Ship, is expressed during hematopoiesis and spermatogenesis. Blood 91: 2753-2759, 1998.; and
Takeshita, S.; Namba, N.; Zhao, J. J.; Jiang, Y.; Genant, H. K.; Silva, M. J.; Brodt, M. D.; Helgason, C. D.; Kalesnikoff, J.; Rauh, M. J.; Humphries, R. K.; Krystal, G.; Teitelbaum, S.

Further studies establishing the function and utilities of INPP5D are found in John Hopkins OMIM database record ID 601582, and in sited publications numbered 1513-1523 listed in the bibliography section hereinbelow, which are also hereby incorporated by reference. Plexin A1 (PLXNA1, Accession XM_{—}051261) is another VGAM292 host target gene. PLXNA1 BINDING SITE is HOST TARGET binding site found in the 3′ untranslated region of mRNA encoded by PLXNA1, corresponding to a HOST TARGET binding site such as BINDING SITE I, BINDING SITE II or BINDING SITE III. Table 2 illustrates the complementarity of the nucleotide sequences of PLXNA1 BINDING SITE, designated SEQ ID:2941, to the nucleotide sequence of VGAM292 RNA, herein designated VGAM RNA, also designated SEQ ID:627.

Another function of VGAM292 is therefore inhibition of Plexin A1 (PLXNA1, Accession XM_{—}051261). Accordingly, utilities of VGAM292 include diagnosis, prevention and treatment of diseases and clinical conditions associated with PLXNA1. Protein Kinase C, Nu (PRKCN, Accession NM_{—}005813) is another VGAM292 host target gene. PRKCN BINDING SITE is HOST TARGET binding site found in the 3′ untranslated region of mRNA encoded by PRKCN, corresponding to a HOST TARGET binding site such as BINDING SITE I, BINDING SITE II or BINDING SITE III. Table 2 illustrates the complementarity of the nucleotide sequences of PRKCN BINDING SITE, designated SEQ ID:1253, to the nucleotide sequence of VGAM292 RNA, herein designated VGAM RNA, also designated SEQ ID:627.

Another function of VGAM292 is therefore inhibition of Protein Kinase C, Nu (PRKCN, Accession NM_{—}005813). Accordingly, utilities of VGAM292 include diagnosis, prevention and treatment of diseases and clinical conditions associated with PRKCN. Betaine-homocysteine Methyltransferase (BHMT, Accession NM_{—}001713) is another VGAM292 host target gene. BHMT BINDING SITE is HOST TARGET binding site found in the 3′ untranslated region of mRNA encoded by BHMT, corresponding to a HOST TARGET binding site such as BINDING SITE I, BINDING SITE II or BINDING SITE III. Table 2 illustrates the complementarity of the nucleotide sequences of BHMT BINDING SITE, designated SEQ ID:849, to the nucleotide sequence of VGAM292 RNA, herein designated VGAM RNA, also designated SEQ ID:627.

Another function of VGAM292 is therefore inhibition of Betaine-homocysteine Methyltransferase (BHMT, Accession NM_{—}001713). Accordingly, utilities of VGAM292 include diagnosis, prevention and treatment of diseases and clinical conditions associated with BHMT. DKFZP434BO44 (Accession NM_{—}031476) is another VGAM292 host target gene. DKFZP434BO44 BINDING SITE is HOST TARGET binding site found in the 3′ untranslated region of mRNA encoded by DKFZP434BO44, corresponding to a HOST TARGET binding site such as BINDING SITE I, BINDING SITE II or BINDING SITE III. Table 2 illustrates the complementarity of the nucleotide sequences of DKFZP434BO44 BINDING SITE, designated SEQ ID:2211, to the nucleotide sequence of VGAM292 RNA, herein designated VGAM RNA, also designated SEQ ID:627.

Another function of VGAM292 is therefore inhibition of DKFZP434BO44 (Accession NM_{—}031476). Accordingly, utilities of VGAM292 include diagnosis, prevention and treatment of diseases and clinical conditions associated with DKFZP434BO44. FLJ10458 (Accession NM_{—}018096) is another VGAM292 host target gene. FLJ10458 BINDING SITE is HOST TARGET binding site found in the 3′ untranslated region of mRNA encoded by FLJ10458, corresponding to a HOST TARGET binding site such as BINDING SITE I, BINDING SITE II or BINDING SITE III. Table 2 illustrates the complementarity of the nucleotide sequences of FLJ10458 BINDING SITE, designated SEQ ID:1777, to the nucleotide sequence of VGAM292 RNA, herein designated VGAM RNA, also designated SEQ ID:627.

Another function of VGAM292 is therefore inhibition of FLJ10458 (Accession NM_{—}018096). Accordingly, utilities of VGAM292 include diagnosis, prevention and treatment of diseases and clinical conditions associated with FLJ10458. FLJ20272 (Accession NM_{—}017735) is another VGAM292 host target gene. FLJ20272 BINDING SITE is HOST TARGET binding site found in the 3′ untranslated region of mRNA encoded by FLJ20272, corresponding to a HOST TARGET binding site such as BINDING SITE I, BINDING SITE II or BINDING SITE III. Table 2 illustrates the complementarity of the nucleotide sequences of FLJ20272 BINDING SITE, designated SEQ ID:1744, to the nucleotide sequence of VGAM292 RNA, herein designated VGAM RNA, also designated SEQ ID:627.

Another function of VGAM292 is therefore inhibition of FLJ20272 (Accession NM_{—}017735). Accordingly, utilities of VGAM292 include diagnosis, prevention and treatment of diseases and clinical conditions associated with FLJ20272. FLJ23598 (Accession XM_{—}170689) is another VGAM292 host target gene. FLJ23598 BINDING SITE is HOST TARGET binding site found in the 3′ untranslated region of mRNA encoded by FLJ23598, corresponding to a HOST TARGET binding site such as BINDING SITE I, BINDING SITE II or BINDING SITE III. Table 2 illustrates the complementarity of the nucleotide sequences of FLJ23598 BINDING SITE, designated SEQ ID:3665, to the nucleotide sequence of VGAM292 RNA, herein designated VGAM RNA, also designated SEQ ID:627.

Another function of VGAM292 is therefore inhibition of FLJ23598 (Accession XM_{—}170689). Accordingly, utilities of VGAM292 include diagnosis, prevention and treatment of diseases and clinical conditions associated with FLJ23598. KIAA1755 (Accession XM_{—}028810) is another VGAM292 host target gene. KIAA1755 BINDING SITE is HOST TARGET binding site found in the 3′ untranslated region of mRNA encoded by KIAA1755, corresponding to a HOST TARGET binding site such as BINDING SITE I, BINDING SITE II or BINDING SITE III. Table 2 illustrates the complementarity of the nucleotide sequences of KIAA1755 BINDING SITE, designated SEQ ID:2595, to the nucleotide sequence of VGAM292 RNA, herein designated VGAM RNA, also designated SEQ ID:627.

Another function of VGAM292 is therefore inhibition of KIAA1755 (Accession XM_{—}028810). Accordingly, utilities of VGAM292 include diagnosis, prevention and treatment of diseases and clinical conditions associated with KIAA1755. MGC21621 (Accession NM_{—}145015) is another VGAM292 host target gene. MGC21621 BINDING SITE is HOST TARGET binding site found in the 3′ untranslated region of mRNA encoded by MGC21621, corresponding to a HOST TARGET binding site such as BINDING SITE I, BINDING SITE II or BINDING SITE III. Table 2 illustrates the complementarity of the nucleotide sequences of MGC21621 BINDING SITE, designated SEQ ID:2508, to the nucleotide sequence of VGAM292 RNA, herein designated VGAM RNA, also designated SEQ ID:627.

Another function of VGAM292 is therefore inhibition of MGC21621 (Accession NM_{—}145015). Accordingly, utilities of VGAM292 include diagnosis, prevention and treatment of diseases and clinical conditions associated with MGC21621. MGC3077 (Accession NM_{—}024051) is another VGAM292 host target gene. MGC3077 BINDING SITE is HOST TARGET binding site found in the 3′ untranslated region of mRNA encoded by MGC3077, corresponding to a HOST TARGET binding site such as BINDING SITE I, BINDING SITE II or BINDING SITE III. Table 2 illustrates the complementarity of the nucleotide sequences of MGC3077 BINDING SITE, designated SEQ ID:2048, to the nucleotide sequence of VGAM292 RNA, herein designated VGAM RNA, also designated SEQ ID:627.

Another function of VGAM292 is therefore inhibition of MGC3077 (Accession NM_{—}024051). Accordingly, utilities of VGAM292 include diagnosis, prevention and treatment of diseases and clinical conditions associated with MGC3077. MGC4707 (Accession NM_{—}024113) is another VGAM292 host target gene. MGC4707 BINDING SITE is HOST TARGET binding site found in the 3′ untranslated region of mRNA encoded by MGC4707, corresponding to a HOST TARGET binding site such as BINDING SITE I, BINDING SITE II or BINDING SITE III. Table 2 illustrates the complementarity of the nucleotide sequences of MGC4707 BINDING SITE, designated SEQ ID:2055, to the nucleotide sequence of VGAM292 RNA, herein designated VGAM RNA, also designated SEQ ID:627.

Another function of VGAM292 is therefore inhibition of MGC4707 (Accession NM_{—}024113). Accordingly, utilities of VGAM292 include diagnosis, prevention and treatment of diseases and clinical conditions associated with MGC4707. MGC5149 (Accession XM_{—}051200) is another VGAM292 host target gene. MGC5149 BINDING SITE is HOST TARGET binding site found in the 3′ untranslated region of mRNA encoded by MGC5149, corresponding to a HOST TARGET binding site such as BINDING SITE I, BINDING SITE II or BINDING SITE III. Table 2 illustrates the complementarity of the nucleotide sequences of MGC5149 BINDING SITE, designated SEQ ID:2940, to the nucleotide sequence of VGAM292 RNA, herein designated VGAM RNA, also designated SEQ ID:627.

Another function of VGAM292 is therefore inhibition of MGC5149 (Accession XM_{—}051200). Accordingly, utilities of VGAM292 include diagnosis, prevention and treatment of diseases and clinical conditions associated with MGC5149. RAB3A Interacting Protein (rabin3)-like 1 (RAB3IL1, Accession NM_{—}013401) is another VGAM292 host target gene. RAB3IL1 BINDING SITE is HOST TARGET binding site found in the 3′ untranslated region of mRNA encoded by RAB3IL1, corresponding to a HOST TARGET binding site such as BINDING SITE I, BINDING SITE II or BINDING SITE III. Table 2 illustrates the complementarity of the nucleotide sequences of RAB31L1 BINDING SITE, designated SEQ ID: 1448, to the nucleotide sequence of VGAM292 RNA, herein designated VGAM RNA, also designated SEQ ID:627.

Another function of VGAM292 is therefore inhibition of RAB3A Interacting Protein (rabin3)-like 1 (RAB3IL1, Accession NM_{—}013401). Accordingly, utilities of VGAM292 include diagnosis, prevention and treatment of diseases and clinical conditions associated with RAB3IL1. LOC119369 (Accession XM_{—}061434) is another VGAM292 host target gene. LOC119369 BINDING SITE is HOST TARGET binding site found in the 5′ untranslated region of mRNA encoded by LOC119369, corresponding to a HOST TARGET binding site such as BINDING SITE I, BINDING SITE II or BINDING SITE III. Table 2 illustrates the complementarity of the nucleotide sequences of LOC119369 BINDING SITE, designated SEQ ID:3026, to the nucleotide sequence of VGAM292 RNA, herein designated VGAM RNA, also designated SEQ ID:627.

Another function of VGAM292 is therefore inhibition of LOC119369 (Accession XM_{—}061434). Accordingly, utilities of VGAM292 include diagnosis, prevention and treatment of diseases and clinical conditions associated with LOC119369. LOC150035 (Accession XM_{—}097793) is another VGAM292 host target gene. LOC150035 BINDING SITE is HOST TARGET binding site found in the 5′ untranslated region of mRNA encoded by LOC150035, corresponding to a HOST TARGET binding site such as BINDING SITE I, BINDING SITE II or BINDING SITE III. Table 2 illustrates the complementarity of the nucleotide sequences of LOC150035 BINDING SITE, designated SEQ ID:3321, to the nucleotide sequence of VGAM292 RNA, herein designated VGAM RNA, also designated SEQ ID:627.

Another function of VGAM292 is therefore inhibition of LOC150035 (Accession XM_{—}097793). Accordingly, utilities of VGAM292 include diagnosis, prevention and treatment of diseases and clinical conditions associated with LOC150035. LOC219649 (Accession XM_{—}167562) is another VGAM292 host target gene. LOC219649 BINDING SITE is HOST TARGET binding site found in the 5′ untranslated region of mRNA encoded by LOC219649, corresponding to a HOST TARGET binding site such as BINDING SITE I, BINDING SITE II or BINDING SITE III. Table 2 illustrates the complementarity of the nucleotide sequences of LOC219649 BINDING SITE, designated SEQ ID:3586, to the nucleotide sequence of VGAM292 RNA, herein designated VGAM RNA, also designated SEQ ID:627.

Another function of VGAM292 is therefore inhibition of LOC219649 (Accession XM_{—}167562). Accordingly, utilities of VGAM292 include diagnosis, prevention and treatment of diseases and clinical conditions associated with LOC219649. LOC221688 (Accession XM_{—}168085) is another VGAM292 host target gene. LOC221688 BINDING SITE is HOST TARGET binding site found in the 5′ untranslated region of mRNA encoded by LOC221688, corresponding to a HOST TARGET binding site such as BINDING SITE I, BINDING SITE II or BINDING SITE III. Table 2 illustrates the complementarity of the nucleotide sequences of LOC221688 BINDING SITE, designated SEQ ID:3617, to the nucleotide sequence of VGAM292 RNA, herein designated VGAM RNA, also designated SEQ ID:627.

Another function of VGAM292 is therefore inhibition of LOC221688 (Accession XM_{—}168085). Accordingly, utilities of VGAM292 include diagnosis, prevention and treatment of diseases and clinical conditions associated with LOC221688. FIG. 1 further provides a conceptual description of a novel bioinformatically detected viral gene of the present invention, referred to here as Viral Genomic Address Messenger 293 (VGAM293) viral gene, which modulates expression of respective host target genes thereof, the function and utility of which host target genes is known in the art.

VGAM293 is a novel bioinformatically detected regulatory, non protein coding, viral micro RNA (miRNA) gene. The method by which VGAM293 was detected is described hereinabove with reference to FIGS. 1-8.

VGAM293 gene, herein designated VGAM GENE, is a viral gene contained in the genome of Vaccinia Virus. VGAM293 host target gene, herein designated VGAM HOST TARGET GENE, is a human gene contained in the human genome.

VGAM293 gene encodes a VGAM293 precursor RNA, herein designated VGAM PRECURSOR RNA. Similar to other miRNA genes, and unlike most ordinary genes, VGAM293 precursor RNA does not encode a protein. A nucleotide sequence identical or highly similar to the nucleotide sequence of VGAM293 precursor RNA is designated SEQ ID:279, and is provided hereinbelow with reference to the sequence listing part. Nucleotide sequence SEQ ID:279 is located at position 165185 relative to the genome of Vaccinia Virus.

VGAM293 precursor RNA folds onto itself, forming VGAM293 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM293 folded precursor RNA into VGAM293 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM293 RNA is designated SEQ ID:628, and is provided hereinbelow with reference to the sequence listing part.

VGAM293 host target gene, herein designated VGAM HOST TARGET GENE, encodes a corresponding messenger RNA, VGAM293 host target RNA, herein designated VGAM HOST TARGET RNA. VGAM293 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM293 RNA, herein designated VGAM RNA, binds complementarily to one or more host target binding sites located in untranslated regions of VGAM293 host target RNA, herein designated VGAM HOST TARGET RNA. This complementary binding is due to the fact that the nucleotide sequence of VGAM293 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM293 RNA, herein designated VGAM RNA, may have a different number of host target binding sites in untranslated regions of a VGAM293 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM293 RNA, herein designated VGAM RNA, to host target binding sites on VGAM293 host target RNA, herein designated VGAM HOST TARGET RNA, such as BINDING SITE I, BINDING SITE II and BINDING SITE III, inhibits translation of VGAM293 host target RNA into VGAM293 host target protein, herein designated VGAM HOST TARGET PROTEIN. VGAM host target protein is therefore outlined by a broken line.

It is appreciated that VGAM293 host target gene, herein designated VGAM HOST TARGET GENE, in fact represents a plurality of VGAM293 host target genes. The mRNA of each one of this plurality of VGAM293 host target genes comprises one or more host target binding sites, each having a nucleotide sequence which is at least partly complementary to VGAM293 RNA, herein designated VGAM RNA, and which when bound by VGAM293 RNA causes inhibition of translation of respective one or more VGAM293 host target proteins.

It is further appreciated by one skilled in the art that the mode of translational inhibition illustrated by FIG. 1 with specific reference to translational inhibition exerted by VGAM293 gene, herein designated VGAM GENE, on one or more VGAM293 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM293 is inhibition of expression of host target genes, as part of a novel viral mechanism of attacking a host. Accordingly, utilities of VGAM293 include diagnosis, prevention and treatment of viral infection by Vaccinia Virus. Specific functions, and accordingly utilities, of VGAM293 correlate with, and may be deduced from, the identity of the host target genes which VGAM293 binds and inhibits, and the function of these host target genes, as elaborated hereinbelow.

Nucleotide sequences of the VGAM293 precursor RNA, herein designated VGAM PRECURSOR RNA, and of the ‘diced’ VGAM293 RNA, herein designated VGAM RNA, and a schematic representation of the secondary folding of VGAM293 folded precursor RNA, herein designated VGAM FOLDED PRECURSOR RNA, of VGAM293 are further described hereinbelow with reference to Table 1.

Nucleotide sequences of host target binding sites, such as BINDING SITE-I, BINDING SITE-II and BINDING SITE-III of FIG. 1, found on VGAM293 host target RNA, and schematic representation of the complementarity of each of these host target binding sites to VGAM293 RNA, herein designated VGAM RNA, are described hereinbelow with reference to Table 2.

As mentioned hereinabove with reference to FIG. 1, a function of VGAM293 gene, herein designated VGAM is inhibition of expression of VGAM293 target genes. It is appreciated that specific functions, and accordingly utilities, of VGAM293 correlate with, and may be deduced from, the identity of the target genes which VGAM293 binds and inhibits, and the function of these target genes, as elaborated hereinbelow.

Retinaldehyde Binding Protein 1 (RLBP1, Accession NM_{—}000326) is a VGAM293 host target gene. RLBP1 BINDING SITE is HOST TARGET binding site found in the 5′ untranslated region of mRNA encoded by RLBP1, corresponding to a HOST TARGET binding site such as BINDING SITE I, BINDING SITE II or BINDING SITE III. Table 2 illustrates the complementarity of the nucleotide sequences of RLBP1 BINDING SITE, designated SEQ ID:729, to the nucleotide sequence of VGAM293 RNA, herein designated VGAM RNA, also designated SEQ ID:628.

A function of VGAM293 is therefore inhibition of Retinaldehyde Binding Protein 1 (RLBP1, Accession NM_{—}000326). Accordingly, utilities of VGAM293 include diagnosis, prevention and treatment of diseases and clinical conditions associated with RLBP1. MGC23280 (Accession NM_{—}144683) is another VGAM293 host target gene. MGC23280 BINDING SITE is HOST TARGET binding site found in the 3′ untranslated region of mRNA encoded by MGC23280, corresponding to a HOST TARGET binding site such as BINDING SITE I, BINDING SITE II or BINDING SITE III. Table 2 illustrates the complementarity of the nucleotide sequences of MGC23280 BINDING SITE, designated SEQ ID:2493, to the nucleotide sequence of VGAM293 RNA, herein designated VGAM RNA, also designated SEQ ID:628.

Another function of VGAM293 is therefore inhibition of MGC23280 (Accession NM_{—}144683). Accordingly, utilities of VGAM293 include diagnosis, prevention and treatment of diseases and clinical conditions associated with MGC23280. LOC113523 (Accession XM_{—}054378) is another VGAM293 host target gene. LOC113523 BINDING SITE is HOST TARGET binding site found in the 3′ untranslated region of mRNA encoded by LOC113523, corresponding to a HOST TARGET binding site such as BINDING SITE I, BINDING SITE II or BINDING SITE III. Table 2 illustrates the complementarity of the nucleotide sequences of LOC113523 BINDING SITE, designated SEQ ID:2966, to the nucleotide sequence of VGAM293 RNA, herein designated VGAM RNA, also designated SEQ ID:628.

Another function of VGAM293 is therefore inhibition of LOC113523 (Accession XM_{—}054378). Accordingly, utilities of VGAM293 include diagnosis, prevention and treatment of diseases and clinical conditions associated with LOC113523. LOC150157 (Accession XM_{—}097823) is another VGAM293 host target gene. LOC150157 BINDING SITE is HOST TARGET binding site found in the 3′ untranslated region of mRNA encoded by LOC150157, corresponding to a HOST TARGET binding site such as BINDING SITE I, BINDING SITE II or BINDING SITE III. Table 2 illustrates the complementarity of the nucleotide sequences of LOC150157 BINDING SITE, designated SEQ ID:3325, to the nucleotide sequence of VGAM293 RNA, herein designated VGAM RNA, also designated SEQ ID:628.

Another function of VGAM293 is therefore inhibition of LOC150157 (Accession XM_{—}097823). Accordingly, utilities of VGAM293 include diagnosis, prevention and treatment of diseases and clinical conditions associated with LOC150157. LOC196890 (Accession XM_{—}116951) is another VGAM293 host target gene. LOC196890 BINDING SITE is HOST TARGET binding site found in the 3′ untranslated region of mRNA encoded by LOC196890, corresponding to a HOST TARGET binding site such as BINDING SITE I, BINDING SITE II or BINDING SITE III. Table 2 illustrates the complementarity of the nucleotide sequences of LOC196890 BINDING SITE, designated SEQ ID:3468, to the nucleotide sequence of VGAM293 RNA, herein designated VGAM RNA, also designated SEQ ID:628.

Another function of VGAM293 is therefore inhibition of LOC196890 (Accession XM_{—}116951). Accordingly, utilities of VGAM293 include diagnosis, prevention and treatment of diseases and clinical conditions associated with LOC196890. FIG. 1 further provides a conceptual description of a novel bioinformatically detected viral gene of the present invention, referred to here as Viral Genomic Address Messenger 294 (VGAM294) viral gene, which modulates expression of respective host target genes thereof, the function and utility of which host target genes is known in the art.

VGAM294 is a novel bioinformatically detected regulatory, non protein coding, viral micro RNA (miRNA) gene. The method by which VGAM294 was detected is described hereinabove with reference to FIGS. 1-8.

VGAM294 gene, herein designated VGAM GENE, is a viral gene contained in the genome of Vaccinia Virus. VGAM294 host target gene, herein designated VGAM HOST TARGET GENE, is a human gene contained in the human genome.

VGAM294 gene encodes a VGAM294 precursor RNA, herein designated VGAM PRECURSOR RNA. Similar to other miRNA genes, and unlike most ordinary genes, VGAM294 precursor RNA does not encode a protein. A nucleotide sequence identical or highly similar to the nucleotide sequence of VGAM294 precursor RNA is designated SEQ ID:280, and is provided hereinbelow with reference to the sequence listing part. Nucleotide sequence SEQ ID:280 is located at position 165538 relative to the genome of Vaccinia Virus.

VGAM294 precursor RNA folds onto itself, forming VGAM294 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM294 folded precursor RNA into VGAM294 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM294 RNA is designated SEQ ID:629, and is provided hereinbelow with reference to the sequence listing part.

VGAM294 host target gene, herein designated VGAM HOST TARGET GENE, encodes a corresponding messenger RNA, VGAM294 host target RNA, herein designated VGAM HOST TARGET RNA. VGAM294 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM294 RNA, herein designated VGAM RNA, binds complementarily to one or more host target binding sites located in untranslated regions of VGAM294 host target RNA, herein designated VGAM HOST TARGET RNA. This complementary binding is due to the fact that the nucleotide sequence of VGAM294 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM294 RNA, herein designated VGAM RNA, may have a different number of host target binding sites in untranslated regions of a VGAM294 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM294 RNA, herein designated VGAM RNA, to host target binding sites on VGAM294 host target RNA, herein designated VGAM HOST TARGET RNA, such as BINDING SITE I, BINDING SITE II and BINDING SITE III, inhibits translation of VGAM294 host target RNA into VGAM294 host target protein, herein designated VGAM HOST TARGET PROTEIN. VGAM host target protein is therefore outlined by a broken line.

It is appreciated that VGAM294 host target gene, herein designated VGAM HOST TARGET GENE, in fact represents a plurality of VGAM294 host target genes. The mRNA of each one of this plurality of VGAM294 host target genes comprises one or more host target binding sites, each having a nucleotide sequence which is at least partly complementary to VGAM294 RNA, herein designated VGAM RNA, and which when bound by VGAM294 RNA causes inhibition of translation of respective one or more VGAM294 host target proteins.

It is further appreciated by one skilled in the art that the mode of translational inhibition illustrated by FIG. 1 with specific reference to translational inhibition exerted by VGAM294 gene, herein designated VGAM GENE, on one or more VGAM294 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM294 is inhibition of expression of host target genes, as part of a novel viral mechanism of attacking a host. Accordingly, utilities of VGAM294 include diagnosis, prevention and treatment of viral infection by Vaccinia Virus. Specific functions, and accordingly utilities, of VGAM294 correlate with, and may be deduced from, the identity of the host target genes which VGAM294 binds and inhibits, and the function of these host target genes, as elaborated hereinbelow.

Nucleotide sequences of the VGAM294 precursor RNA, herein designated VGAM PRECURSOR RNA, and of the ‘diced’ VGAM294 RNA, herein designated VGAM RNA, and a schematic representation of the secondary folding of VGAM294 folded precursor RNA, herein designated VGAM FOLDED PRECURSOR RNA, of VGAM294 are further described hereinbelow with reference to Table 1.

Nucleotide sequences of host target binding sites, such as BINDING SITE-I, BINDING SITE-II and BINDING SITE-III of FIG. 1, found on VGAM294 host target RNA, and schematic representation of the complementarity of each of these host target binding sites to VGAM294 RNA, herein designated VGAM RNA, are described hereinbelow with reference to Table 2.

As mentioned hereinabove with reference to FIG. 1, a function of VGAM294 gene, herein designated VGAM is inhibition of expression of VGAM294 target genes. It is appreciated that specific functions, and accordingly utilities, of VGAM294 correlate with, and may be deduced from, the identity of the target genes which VGAM294 binds and inhibits, and the function of these target genes, as elaborated hereinbelow.

Rho GTPase Activating Protein 6 (ARHGAP6, Accession NM_{—}001174) is a VGAM294 host target gene. ARHGAP6 BINDING SITE1 and ARHGAP6 BINDING SITE2 are HOST TARGET binding sites found in untranslated regions of mRNA encoded by ARHGAP6, corresponding to HOST TARGET binding sites such as BINDING SITE I, BINDING SITE II or BINDING SITE III. Table 2 illustrates the complementarity of the nucleotide sequences of ARHGAP6 BINDING SITE1 and ARHGAP6 BINDING SITE2, designated SEQ ID:807 and SEQ ID: 1450 respectively, to the nucleotide sequence of VGAM294 RNA, herein designated VGAM RNA, also designated SEQ ID:629.

A function of VGAM294 is therefore inhibition of Rho GTPase Activating Protein 6 (ARHGAP6, Accession NM_{—}001174), a gene which activates the rho-type GTPases by converting them to an inactive GTP-bound state. Accordingly, utilities of VGAM294 include diagnosis, prevention and treatment of diseases and clinical conditions associated with ARHGAP6. The function of ARHGAP6 has been established by previous studies. In a search for the genetic defect in microphthalmia with linear skin defects syndrome (MLS; 309801), Schaefer et al. (1997) trapped exons from 14 overlapping cosmids from the 500-kb MLS critical region in Xp22.3. Using exon connection followed by cDNA library screening, they identified a 2.4-kb contig of cDNA clones spanning 170 kb of genomic sequence in the MLS deletion region. Northern analysis of this cDNA detected a prominent transcript of approximately 4.2 kb and a less abundant transcript of approximately 6 kb in all tissues examined, with additional transcripts in skeletal muscle. Sequence analysis revealed a coding region of 601 amino acids contained in 12 exons, with a splice variant isoform of 495 amino acids. The predicted protein sequence of the gene, symbolized ARHGAP6, contains homology to the GTPase-activating (GAP) domain of the Rho-GAP family of proteins (e.g., 300023), which has been implicated in the regulation of actin polymerization at the plasma membrane in several cellular processes. Schaefer et al. (1997) discussed reasons for thinking that a defect in the Rho pathway may play a role in the pathogenesis of MLS syndrome. Prakash et al. (2000) investigated the function of ARHGAP6 by generating Arhgap6 null mice and also by in vitro expression studies. Surprisingly, loss of the rhoGAP function of Arhgap6 did not cause any detectable phenotypic or behavioral abnormalities in the mutant mice. Transfected mammalian cells expressing ARHGAP6 lost their actin stress fibers, retracted from the growth surface, and extended thin, branching processes resembling filopodia. The ARHGAP6 protein colocalized with actin filaments through an N-terminal domain and recruited filamentous actin into the growing processes. Mutation of a conserved arginine residue in the rhoGAP domain prevented the loss of stress fibers but had little effect on process outgrowth. The authors concluded that ARHGAP6 has 2 independent functions: one as a GAP with specificity for RhoA and the other as a cytoskeletal protein that promotes actin remodeling.

Full details of the abovementioned studies are described in the following publications, the disclosure of which are hereby incorporated by reference:
Prakash, S. K.; Paylor, R.; Jenna, S.; Lamarche-Vane, N.; Armstrong, D. L.; Xu, B.; Mancini, M. A.; Zoghbi, H. Y.: Functional analysis of ARHGAP6, a novel GTPase-activating protein for RhoA. Hum. Molec. Genet. 9: 477-488, 2000.; and
Schaefer, L.; Prakash, S.; Zoghbi, H. Y.: Cloning and characterization of a novel rho-type GTPase-activating protein gene (ARHGAP6) from the critical region for microphthalmia with Ii.

Further studies establishing the function and utilities of ARHGAP6 are found in John Hopkins OMIM database record ID 300118, and in sited publications numbered 2432-2433 listed in the bibliography section hereinbelow, which are also hereby incorporated by reference. Basonuclin (BNC, Accession NM_{—}001717) is another VGAM294 host target gene. BNC BINDING SITE is HOST TARGET binding site found in the 3′ untranslated region of mRNA encoded by BNC, corresponding to a HOST TARGET binding site such as BINDING SITE I, BINDING SITE II or BINDING SITE III. Table 2 illustrates the complementarity of the nucleotide sequences of BNC BINDING SITE, designated SEQ ID:850, to the nucleotide sequence of VGAM294 RNA, herein designated VGAM RNA, also designated SEQ ID:629.

Another function of VGAM294 is therefore inhibition of Basonuclin (BNC, Accession NM_{—}001717), a gene which plays a role in the maintenance of proliferative capacity and prevention of terminal differentiation of keratinocytes. Accordingly, utilities of VGAM294 include diagnosis, prevention and treatment of diseases and clinical conditions associated with BNC. The function of BNC and its association with various diseases and clinical conditions, has been established by previous studies, as described hereinabove with reference to VGAM274. CAMP Responsive Element Binding Protein-like 2 (CREBL2, Accession NM_{—}001310) is another VGAM294 host target gene. CREBL2 BINDING SITE is HOST TARGET binding site found in the 3′ untranslated region of mRNA encoded by CREBL2, corresponding to a HOST TARGET binding site such as BINDING SITE I, BINDING SITE II or BINDING SITE III. Table 2 illustrates the complementarity of the nucleotide sequences of CREBL2 BINDING SITE, designated SEQ ID:817, to the nucleotide sequence of VGAM294 RNA, herein designated VGAM RNA, also designated SEQ ID:629.

Another function of VGAM294 is therefore inhibition of CAMP Responsive Element Binding Protein-like 2 (CREBL2, Accession NM_{—}001310). Accordingly, utilities of VGAM294 include diagnosis, prevention and treatment of diseases and clinical conditions associated with CREBL2. Glucosaminyl (N-acetyl) Transferase 1, Core 2 (beta-1,6-N-acetylglucosaminyltransferase) (GCNT1, Accession NM_{—}001490) is another VGAM294 host target gene. GCNT1 BINDING SITE is HOST TARGET binding site found in the 3′ untranslated region of mRNA encoded by GCNT1, corresponding to a HOST TARGET binding site such as BINDING SITE I, BINDING SITE II or BINDING SITE III. Table 2 illustrates the complementarity of the nucleotide sequences of GCNT1 BINDING SITE, designated SEQ ID:830, to the nucleotide sequence of VGAM294 RNA, herein designated VGAM RNA, also designated SEQ ID:629.

Another function of VGAM294 is therefore inhibition of Glucosaminyl (N-acetyl) Transferase 1, Core 2 (beta-1,6-N-acetylglucosaminyltransferase) (GCNT1, Accession NM_{—}001490), a gene which forms critical branches in o-glycans. Accordingly, utilities of VGAM294 include diagnosis, prevention and treatment of diseases and clinical conditions associated with GCNT1. The function of GCNT1 has been established by previous studies. Bierhuizen et al. (1993) provided the sequence of the developmental I antigen encoded by the cDNA for a member of a beta-1,6-N-acetylglucosaminyltransferase gene family. By Northern blot analysis, Yeh et al. (1999) showed that multiple transcripts of GCNT1 were expressed in nearly all tissues tested, whereas expression of GCNT3 (OMIM Ref. No. 606836) was more restricted. Transcripts were also readily detected in some leukemic cell lines and in colon and cervical carcinoma cell lines, but not in a lung carcinoma cell line.

Full details of the abovementioned studies are described in the following publications, the disclosure of which are hereby incorporated by reference:
Bierhuizen, M. F. A.; Mattei, M.-G.; Fukuda, M.: Expression of the developmental I antigen by a cloned human cDNA encoding a member of a beta-1,6-N-acetylglucosaminyltransferase gene family. Genes Dev. 7: 468-478, 1993.; and
Pilz, A.; Woodward, K.; Povey, S.; Abbott, C.: Comparative mapping of 50 human chromosome 9 loci in the laboratory mouse. Genomics 25: 139-149, 1995.

Further studies establishing the function and utilities of GCNT1 are found in John Hopkins OMIM database record ID 600391, and in sited publications numbered 282 and 2831 listed in the bibliography section hereinbelow, which are also hereby incorporated by reference. Muscleblind-like (Drosophila) (MBNL, Accession NM_{—}021038) is another VGAM294 host target gene. MBNL BINDING SITE is HOST TARGET binding site found in the 3′ untranslated region of mRNA encoded by MBNL, corresponding to a HOST TARGET binding site such as BINDING SITE I, BINDING SITE II or BINDING SITE III. Table 2 illustrates the complementarity of the nucleotide sequences of MBNL BINDING SITE, designated SEQ ID:1938, to the nucleotide sequence of VGAM294 RNA, herein designated VGAM RNA, also designated SEQ ID:629.

Another function of VGAM294 is therefore inhibition of Muscleblind-like (Drosophila) (MBNL, Accession NM_{—}021038), a gene which binds to cug triplet repeat expansion dsrna (by similarity). Accordingly, utilities of VGAM294 include diagnosis, prevention and treatment of diseases and clinical conditions associated with MBNL. The function of MBNL and its association with various diseases and clinical conditions, has been established by previous studies, as described hereinabove with reference to VGAM196. AFAP (Accession NM_{—}021638) is another VGAM294 host target gene. AFAP BINDING SITE is HOST TARGET binding site found in the 3′ untranslated region of mRNA encoded by AFAP, corresponding to a HOST TARGET binding site such as BINDING SITE I, BINDING SITE II or BINDING SITE III. Table 2 illustrates the complementarity of the nucleotide sequences of AFAP BINDING SITE, designated SEQ ID:1952, to the nucleotide sequence of VGAM294 RNA, herein designated VGAM RNA, also designated SEQ ID:629.

Another function of VGAM294 is therefore inhibition of AFAP (Accession NM_{—}021638). Accordingly, utilities of VGAM294 include diagnosis, prevention and treatment of diseases and clinical conditions associated with AFAP. LOC148195 (Accession XM_{—}097419) is another VGAM294 host target gene. LOC148195 BINDING SITE is HOST TARGET binding site found in the 3′ untranslated region of mRNA encoded by LOC148195, corresponding to a HOST TARGET binding site such as BINDING SITE I, BINDING SITE II or BINDING SITE III. Table 2 illustrates the complementarity of the nucleotide sequences of LOC148195 BINDING SITE, designated SEQ ID:3299, to the nucleotide sequence of VGAM294 RNA, herein designated VGAM RNA, also designated SEQ ID:629.

Another function of VGAM294 is therefore inhibition of LOC148195 (Accession XM_{—}097419). Accordingly, utilities of VGAM294 include diagnosis, prevention and treatment of diseases and clinical conditions associated with LOC148195. FIG. 1 further provides a conceptual description of a novel bioinformatically detected viral gene of the present invention, referred to here as Viral Genomic Address Messenger 295 (VGAM295) viral gene, which modulates expression of respective host target genes thereof, the function and utility of which host target genes is known in the art.

VGAM295 is a novel bioinformatically detected regulatory, non protein coding, viral micro RNA (miRNA) gene. The method by which VGAM295 was detected is described hereinabove with reference to FIGS. 1-8.

VGAM295 gene, herein designated VGAM GENE, is a viral gene contained in the genome of Vaccinia Virus. VGAM295 host target gene, herein designated VGAM HOST TARGET GENE, is a human gene contained in the human genome.

VGAM295 gene encodes a VGAM295 precursor RNA, herein designated VGAM PRECURSOR RNA. Similar to other miRNA genes, and unlike most ordinary genes, VGAM295 precursor RNA does not encode a protein. A nucleotide sequence identical or highly similar to the nucleotide sequence of VGAM295 precursor RNA is designated SEQ ID:281, and is provided hereinbelow with reference to the sequence listing part. Nucleotide sequence SEQ ID:281 is located at position 172682 relative to the genome of Vaccinia Virus.

VGAM295 precursor RNA folds onto itself, forming VGAM295 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM295 folded precursor RNA into VGAM295 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM295 RNA is designated SEQ ID:630, and is provided hereinbelow with reference to the sequence listing part.

VGAM295 host target gene, herein designated VGAM HOST TARGET GENE, encodes a corresponding messenger RNA, VGAM295 host target RNA, herein designated VGAM HOST TARGET RNA. VGAM295 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM295 RNA, herein designated VGAM RNA, binds complementarily to one or more host target binding sites located in untranslated regions of VGAM295 host target RNA, herein designated VGAM HOST TARGET RNA. This complementary binding is due to the fact that the nucleotide sequence of VGAM295 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM295 RNA, herein designated VGAM RNA, may have a different number of host target binding sites in untranslated regions of a VGAM295 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM295 RNA, herein designated VGAM RNA, to host target binding sites on VGAM295 host target RNA, herein designated VGAM HOST TARGET RNA, such as BINDING SITE I, BINDING SITE II and BINDING SITE III, inhibits translation of VGAM295 host target RNA into VGAM295 host target protein, herein designated VGAM HOST TARGET PROTEIN. VGAM host target protein is therefore outlined by a broken line.

It is appreciated that VGAM295 host target gene, herein designated VGAM HOST TARGET GENE, in fact represents a plurality of VGAM295 host target genes. The mRNA of each one of this plurality of VGAM295 host target genes comprises one or more host target binding sites, each having a nucleotide sequence which is at least partly complementary to VGAM295 RNA, herein designated VGAM RNA, and which when bound by VGAM295 RNA causes inhibition of translation of respective one or more VGAM295 host target proteins.

It is further appreciated by one skilled in the art that the mode of translational inhibition illustrated by FIG. 1 with specific reference to translational inhibition exerted by VGAM295 gene, herein designated VGAM GENE, on one or more VGAM295 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM295 is inhibition of expression of host target genes, as part of a novel viral mechanism of attacking a host. Accordingly, utilities of VGAM295 include diagnosis, prevention and treatment of viral infection by Vaccinia Virus. Specific functions, and accordingly utilities, of VGAM295 correlate with, and may be deduced from, the identity of the host target genes which VGAM295 binds and inhibits, and the function of these host target genes, as elaborated hereinbelow.

Nucleotide sequences of the VGAM295 precursor RNA, herein designated VGAM PRECURSOR RNA, and of the ‘diced’ VGAM295 RNA, herein designated VGAM RNA, and a schematic representation of the secondary folding of VGAM295 folded precursor RNA, herein designated VGAM FOLDED PRECURSOR RNA, of VGAM295 are further described hereinbelow with reference to Table 1.

Nucleotide sequences of host target binding sites, such as BINDING SITE-I, BINDING SITE-II and BINDING SITE-III of FIG. 1, found on VGAM295 host target RNA, and schematic representation of the complementarity of each of these host target binding sites to VGAM295 RNA, herein designated VGAM RNA, are described hereinbelow with reference to Table 2.

As mentioned hereinabove with reference to FIG. 1, a function of VGAM295 gene, herein designated VGAM is inhibition of expression of VGAM295 target genes. It is appreciated that specific functions, and accordingly utilities, of VGAM295 correlate with, and may be deduced from, the identity of the target genes which VGAM295 binds and inhibits, and the function of these target genes, as elaborated hereinbelow.

ATPase, H+ Transporting, Lysosomal 70 kDa, V1 Subunit A, Isoform 1 (ATP6V1A1, Accession NM_{—}001690) is a VGAM295 host target gene. ATP6V1A1 BINDING SITE is HOST TARGET binding site found in the 3′ untranslated region of mRNA encoded by ATP6V1A1, corresponding to a HOST TARGET binding site such as BINDING SITE I, BINDING SITE II or BINDING SITE III. Table 2 illustrates the complementarity of the nucleotide sequences of ATP6V1A1 BINDING SITE, designated SEQ ID:847, to the nucleotide sequence of VGAM295 RNA, herein designated VGAM RNA, also designated SEQ ID:630.

A function of VGAM295 is therefore inhibition of ATPase, H+ Transporting, Lysosomal 70 kDa, V1 Subunit A, Isoform 1 (ATP6V1A1, Accession NM_{—}001690), a gene which is responsible for acidifying a variety of intracellular compartments in eukaryotic cells. Accordingly, utilities of VGAM295 include diagnosis, prevention and treatment of diseases and clinical conditions associated with ATP6V1A1. The function of ATP6V1A1 has been established by previous studies. Van Hille et al. (1993) cloned a partial cDNA clone for an A subunit isoform, which they designated VA68, from a human osteoclastoma tumor cDNA library by PCR using degenerate primers based on the bovine sequence. They obtained a full-length clone from a genomic library. The deduced 617-amino acid protein has a predicted molecular mass of about 68 kD and shows 99% sequence identity with the bovine brain subunit A. Northern blot analysis revealed ubiquitous expression of a major 4.8-kb band and a minor 3.4-kb band. They also identified a variant, which they designated H068, encoding a 615-amino acid protein. By RNase protection assays and in situ hybridization, van Hille et al. (1995) determined that expression of the H068 variant was specific to the osteoclastoma originally used to construct the cDNA library.

Full details of the abovementioned studies are described in the following publications, the disclosure of which are hereby incorporated by reference:
van Hille, B.; Richener, H.; Evans, D. B.; Green, J. R.; Bilbe, G.: Identification of two subunit A isoforms of the vacuolar H(+)-ATPase in human osteoclastoma. J. Biol. Chem. 268: 7075-7080, 1993.; and
van Hille, B.; Richener, H.; Green, J. R.; Bilbe, G.: The ubiquitous VA68 isoform of subunit A of the vacuolar H(+)-ATPase is highly expressed in human osteoclasts. Biochem. Biophys.

Further studies establishing the function and utilities of ATP6V1A1 are found in John Hopkins OMIM database record ID 607027, and in sited publications numbered 1226-1227 listed in the bibliography section hereinbelow, which are also hereby incorporated by reference. Death-associated Protein (DAP, Accession XM_{—}051902) is another VGAM295 host target gene. DAP BINDING SITE is HOST TARGET binding site found in the 3′ untranslated region of mRNA encoded by DAP, corresponding to a HOST TARGET binding site such as BINDING SITE I, BINDING SITE II or BINDING SITE III. Table 2 illustrates the complementarity of the nucleotide sequences of DAP BINDING SITE, designated SEQ ID:2954, to the nucleotide sequence of VGAM295 RNA, herein designated VGAM RNA, also designated SEQ ID:630.

Another function of VGAM295 is therefore inhibition of Death-associated Protein (DAP, Accession XM_{—}051902), a gene which mediates in programmed cell death. Accordingly, utilities of VGAM295 include diagnosis, prevention and treatment of diseases and clinical conditions associated with DAP. The function of DAP has been established by previous studies. Deiss et al. (1995) reported that gamma-interferon (OMIM Ref. No. 147570) induced in HeLa cells a type of death that had cytologic characteristics of programmed cell death. In this system, they identified 2 novel genes whose expression was indispensable for the execution of this type of cell death. The rescue was based on positive growth selection of cells after transfection with antisense cDNA expression libraries. The antisense RNA-mediated inactivation of the 2 novel genes protected the cells from the IFNG-induced cell death, but not from the cytostatic effects of the cytokine or from a necrotic type of cell death. One of these genes was death-associated protein kinase-1 (OMIM Ref. No. 600831). The other, designated death-associated protein (DAP), was expressed as a single 2.4-kb mRNA that encodes a basic, proline-rich, 15-kD protein. The expression levels of the 2 proteins were selectively reduced by the corresponding antisense RNAs. The authors suggested that these 2 novel genes are candidates for positive mediators of programmed cell death that is induced by interferon-gamma. By analysis of rodent-human somatic cell hybrids, Feinstein et al. (1995) found that DAP is located on chromosome 5p15.2 in a region just centromeric to that to which the cri-du-chat syndrome (OMIM Ref. No. 123450) has been mapped

Full details of the abovementioned studies are described in the following publications, the disclosure of which are hereby incorporated by reference:
Deiss, L. P.; Feinstein, E.; Berissi, H.; Cohen, O.; Kimchi, A.: Identification of a novel serine/threonine kinase and a novel 15-kD protein as potential mediators of the gamma interferon-induced cell death. Genes Dev. 9: 15-30, 1995.; and
Feinstein, E.; Druck, T.; Kastury, K.; Berissi, H.; Goodart, S. A.; Overhauser, J.; Kimchi, A.; Huebner, K.: Assignment of DAP1 and DAPK: genes that positively mediate programmed cell.

Further studies establishing the function and utilities of DAP are found in John Hopkins OMIM database record ID 600954, and in sited publications numbered 1877-1878 listed in the bibliography section hereinbelow, which are also hereby incorporated by reference. Leukemia Inhibitory Factor Receptor (LIFR, Accession NM_{—}002310) is another VGAM295 host target gene. LIFR BINDING SITE is HOST TARGET binding site found in the 3′ untranslated region of mRNA encoded by LIFR, corresponding to a HOST TARGET binding site such as BINDING SITE I, BINDING SITE II or BINDING SITE III. Table 2 illustrates the complementarity of the nucleotide sequences of LIFR BINDING SITE, designated SEQ ID:919, to the nucleotide sequence of VGAM295 RNA, herein designated VGAM RNA, also designated SEQ ID:630.

Another function of VGAM295 is therefore inhibition of Leukemia Inhibitory Factor Receptor (LIFR, Accession NM_{—}002310). Accordingly, utilities of VGAM295 include diagnosis, prevention and treatment of diseases and clinical conditions associated with LIFR. Reversion-inducing-cysteine-rich Protein with Kazal Motifs (RECK, Accession NM_{—}021111) is another VGAM295 host target gene. RECK BINDING SITE is HOST TARGET binding site found in the 3′ untranslated region of mRNA encoded by RECK, corresponding to a HOST TARGET binding site such as BINDING SITE I, BINDING SITE II or BINDING SITE III. Table 2 illustrates the complementarity of the nucleotide sequences of RECK BINDING SITE, designated SEQ ID:1941, to the nucleotide sequence of VGAM295 RNA, herein designated VGAM RNA, also designated SEQ ID:630.

Another function of VGAM295 is therefore inhibition of Reversion-inducing-cysteine-rich Protein with Kazal Motifs (RECK, Accession NM_{—}021111), a gene which plays a role in regulation of cancer progression and tumor angiogenesis. Accordingly, utilities of VGAM295 include diagnosis, prevention and treatment of diseases and clinical conditions associated with RECK. The function of RECK has been established by previous studies. Transformed malignant cell lines frequently lose a flat morphology and acquire a round morphology. Genes that induce flat reversion may be useful in the control of cancer. By screening a fibroblast expression library for reversion-inducing cDNAs, Takahashi et al. (1998) isolated a cDNA encoding RECK (reversion-inducing, cysteine-rich protein with Kazal motifs). Sequence analysis predicted that the 971-amino acid RECK protein, which shares 93% amino acid identity with mouse Reck, is 9% cysteine and contains an N-terminal signal sequence; 5 putative cysteine knot motifs; 5 potential N-glycosylation sites; 3 central serine protease inhibitor domains with either complete or incomplete Kazal-type, 4-cys motifs; 2 regions with weak homology to EGF-like repeats; and a C-terminal hydrophobic glycosylphosphatidylinositol-anchoring signal. Immunoblot analysis showed that RECK is expressed as a 110-kD protein that is reduced to approximately 100 kD after deglycosylation. Northern blot analysis detected a 4.6-kb RECK transcript in a wide variety of tissues and normal cell lines, but no expression was detected in tumor cell lines. Restoration of RECK expression in tumor cell lines did not affect growth but did significantly suppress matrix invasion and metastatic activity. SDS-PAGE and gelatin zymography analysis demonstrated that due to a posttranscriptional event(s), secretion of MMP9 (OMIM Ref. No. 120361), a key enzyme in tumor invasion and metastasis, is decreased in cells expressing RECK. An RECK mutant lacking the C-terminal 23 residues retained the ability to suppress tumor cell invasion and MMP9 proteolytic activity but lost the ability to inhibit MMP9 release. Animal model experiments lend further support to the function of RECK. Oh et al. (2001) showed that in addition to MMP9, RECK also regulates MMP2 (OMIM Ref. No. 120360) and MT1-MMP (MMP14; 600754), which are known to be involved in cancer progression. Mice lacking a functional Reck gene died around embryonic day 10.5 with defects in collagen fibrils, the basal lamina, and vascular development; this phenotype could be partially suppressed by Mmp2 null mutation. Vascular sprouting was dramatically suppressed in tumors derived from Reck-expressing fibrosarcoma cells grown in nude mice. These results supported a role for RECK in the regulation of MMP2 in vivo and implicated RECK downregulation in tumor angiogenesis.

It is appreciated that the abovementioned animal model for RECK is acknowledged by those skilled in the art as a scientifically valid animal model, as can be further appreciated from the publications sited hereinbelow.

Full details of the abovementioned studies are described in the following publications, the disclosure of which are hereby incorporated by reference:
Takahashi, C.; Sheng, Z.; Horan, T. P.; Kitayama, H.; Maki, M.; Hitomi, K.; Kitaura, Y.; Takai, S.; Sasahara, R. M.; Horimoto, A.; Ikawa, Y.; Ratzkin, B. J.; Arakawa, T.; Noda, M.: Regulation of matrix metalloproteinase-9 and inhibition of tumor invasion by the membrane-anchored glycoprotein RECK. Proc. Nat. Acad. Sci. 95: 13221-13226, 1998.; and
Oh, J.; Takahashi, R.; Kondo, S.; Mizoguchi, A.; Adachi, E.; Sasahara, R. M.; Nishimura, S.; Imamura, Y.; Kitayama, H.; Alexander, D. B.; Ide, C.; Horan, T. P.; Arakawa, T.; Yoshida, H.

Further studies establishing the function and utilities of RECK are found in John Hopkins OMIM database record ID 605227, and in sited publications numbered 1654-1655 listed in the bibliography section hereinbelow, which are also hereby incorporated by reference. Solute Carrier Family 13 (sodium-dependent dicarboxylate transporter), Member 3 (SLC13A3, Accession XM_{—}017841) is another VGAM295 host target gene. SLC13A3 BINDING SITE is HOST TARGET binding site found in the 3′ untranslated region of mRNA encoded by SLC13A3, corresponding to a HOST TARGET binding site such as BINDING SITE I, BINDING SITE II or BINDING SITE III. Table 2 illustrates the complementarity of the nucleotide sequences of SLC13A3 BINDING SITE, designated SEQ ID:2570, to the nucleotide sequence of VGAM295 RNA, herein designated VGAM RNA, also designated SEQ ID:630.

Another function of VGAM295 is therefore inhibition of Solute Carrier Family 13 (sodium-dependent dicarboxylate transporter), Member 3 (SLC13A3, Accession XM_{—}017841). Accordingly, utilities of VGAM295 include diagnosis, prevention and treatment of diseases and clinical conditions associated with SLC13A3. TEM8 (Accession NM_{—}032208) is another VGAM295 host target gene. TEM8 BINDING SITE is HOST TARGET binding site found in the 3′ untranslated region of mRNA encoded by TEM8, corresponding to a HOST TARGET binding site such as BINDING SITE I, BINDING SITE II or BINDING SITE III. Table 2 illustrates the complementarity of the nucleotide sequences of TEM8 BINDING SITE, designated SEQ ID:2236, to the nucleotide sequence of VGAM295 RNA, herein designated VGAM RNA, also designated SEQ ID:630.

Another function of VGAM295 is therefore inhibition of TEM8 (Accession NM_{—}032208), a gene which is a tumor-specific endothelial marker. Accordingly, utilities of VGAM295 include diagnosis, prevention and treatment of diseases and clinical conditions associated with TEM8. The function of TEM8 and its association with various diseases and clinical conditions, has been established by previous studies, as described hereinabove with reference to VGAM135. DKFZp5471224 (Accession NM_{—}020221) is another VGAM295 host target gene. DKFZp5471224 BINDING SITE is HOST TARGET binding site found in the 3′ untranslated region of mRNA encoded by DKFZp5471224, corresponding to a HOST TARGET binding site such as BINDING SITE I, BINDING SITE II or BINDING SITE III. Table 2 illustrates the complementarity of the nucleotide sequences of DKFZp5471224 BINDING SITE, designated SEQ ID:1903, to the nucleotide sequence of VGAM295 RNA, herein designated VGAM RNA, also designated SEQ ID:630.

Another function of VGAM295 is therefore inhibition of DKFZp5471224 (Accession NM_{—}020221). Accordingly, utilities of VGAM295 include diagnosis, prevention and treatment of diseases and clinical conditions associated with DKFZp5471224. Olfactomedin 3 (OLFM3, Accession XM_{—}088951) is another VGAM295 host target gene. OLFM3 BINDING SITE is HOST TARGET binding site found in the 3′ untranslated region of mRNA encoded by OLFM3, corresponding to a HOST TARGET binding site such as BINDING SITE I, BINDING SITE II or BINDING SITE III. Table 2 illustrates the complementarity of the nucleotide sequences of OLFM3 BINDING SITE, designated SEQ ID:3232, to the nucleotide sequence of VGAM295 RNA, herein designated VGAM RNA, also designated SEQ ID:630.

Another function of VGAM295 is therefore inhibition of Olfactomedin 3 (OLFM3, Accession XM_{—}088951). Accordingly, utilities of VGAM295 include diagnosis, prevention and treatment of diseases and clinical conditions associated with OLFM3. TSARG1 (Accession NM_{—}139073) is another VGAM295 host target gene. TSARG1 BINDING SITE is HOST TARGET binding site found in the 3′ untranslated region of mRNA encoded by TSARG1, corresponding to a HOST TARGET binding site such as BINDING SITE I, BINDING SITE II or BINDING SITE III. Table 2 illustrates the complementarity of the nucleotide sequences of TSARG1 BINDING SITE, designated SEQ ID:2470, to the nucleotide sequence of VGAM295 RNA, herein designated VGAM RNA, also designated SEQ ID:630.

Another function of VGAM295 is therefore inhibition of TSARG1 (Accession NM_{—}139073). Accordingly, utilities of VGAM295 include diagnosis, prevention and treatment of diseases and clinical conditions associated with TSARG1. LOC114987 (Accession NM_{—}145241) is another VGAM295 host target gene. LOC114987 BINDING SITE is HOST TARGET binding site found in the 3′ untranslated region of mRNA encoded by LOC114987, corresponding to a HOST TARGET binding site such as BINDING SITE I, BINDING SITE II or BINDING SITE III. Table 2 illustrates the complementarity of the nucleotide sequences of LOC114987 BINDING SITE, designated SEQ ID:2517, to the nucleotide sequence of VGAM295 RNA, herein designated VGAM RNA, also designated SEQ ID:630.

Another function of VGAM295 is therefore inhibition of LOC114987 (Accession NM_{—}145241). Accordingly, utilities of VGAM295 include diagnosis, prevention and treatment of diseases and clinical conditions associated with LOC114987. LOC152445 (Accession XM_{—}098231) is another VGAM295 host target gene. LOC152445 BINDING SITE is HOST TARGET binding site found in the 5′ untranslated region of mRNA encoded by LOC152445, corresponding to a HOST TARGET binding site such as BINDING SITE I, BINDING SITE II or BINDING SITE III. Table 2 illustrates the complementarity of the nucleotide sequences of LOC152445 BINDING SITE, designated SEQ ID:3360, to the nucleotide sequence of VGAM295 RNA, herein designated VGAM RNA, also designated SEQ ID:630.

Another function of VGAM295 is therefore inhibition of LOC152445 (Accession XM_{—}098231). Accordingly, utilities of VGAM295 include diagnosis, prevention and treatment of diseases and clinical conditions associated with LOC152445. FIG. 1 further provides a conceptual description of a novel bioinformatically detected viral gene of the present invention, referred to here as Viral Genomic Address Messenger 296 (VGAM296) viral gene, which modulates expression of respective host target genes thereof, the function and utility of which host target genes is known in the art.

VGAM296 is a novel bioinformatically detected regulatory, non protein coding, viral micro RNA (miRNA) gene. The method by which VGAM296 was detected is described hereinabove with reference to FIGS. 1-8.

VGAM296 gene, herein designated VGAM GENE, is a viral gene contained in the genome of Vaccinia Virus. VGAM296 host target gene, herein designated VGAM HOST TARGET GENE, is a human gene contained in the human genome.

VGAM296 gene encodes a VGAM296 precursor RNA, herein designated VGAM PRECURSOR RNA. Similar to other miRNA genes, and unlike most ordinary genes, VGAM296 precursor RNA does not encode a protein. A nucleotide sequence identical or highly similar to the nucleotide sequence of VGAM296 precursor RNA is designated SEQ ID:282, and is provided hereinbelow with reference to the sequence listing part. Nucleotide sequence SEQ ID:282 is located at position 169865 relative to the genome of Vaccinia Virus.

VGAM296 precursor RNA folds onto itself, forming VGAM296 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM296 folded precursor RNA into VGAM296 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM296 RNA is designated SEQ ID:631, and is provided hereinbelow with reference to the sequence listing part.

VGAM296 host target gene, herein designated VGAM HOST TARGET GENE, encodes a corresponding messenger RNA, VGAM296 host target RNA, herein designated VGAM HOST TARGET RNA. VGAM296 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM296 RNA, herein designated VGAM RNA, binds complementarily to one or more host target binding sites located in untranslated regions of VGAM296 host target RNA, herein designated VGAM HOST TARGET RNA. This complementary binding is due to the fact that the nucleotide sequence of VGAM296 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM296 RNA, herein designated VGAM RNA, may have a different number of host target binding sites in untranslated regions of a VGAM296 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM296 RNA, herein designated VGAM RNA, to host target binding sites on VGAM296 host target RNA, herein designated VGAM HOST TARGET RNA, such as BINDING SITE I, BINDING SITE II and BINDING SITE III, inhibits translation of VGAM296 host target RNA into VGAM296 host target protein, herein designated VGAM HOST TARGET PROTEIN. VGAM host target protein is therefore outlined by a broken line.

It is appreciated that VGAM296 host target gene, herein designated VGAM HOST TARGET GENE, in fact represents a plurality of VGAM296 host target genes. The mRNA of each one of this plurality of VGAM296 host target genes comprises one or more host target binding sites, each having a nucleotide sequence which is at least partly complementary to VGAM296 RNA, herein designated VGAM RNA, and which when bound by VGAM296 RNA causes inhibition of translation of respective one or more VGAM296 host target proteins.

It is further appreciated by one skilled in the art that the mode of translational inhibition illustrated by FIG. 1 with specific reference to translational inhibition exerted by VGAM296 gene, herein designated VGAM GENE, on one or more VGAM296 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM296 is inhibition of expression of host target genes, as part of a novel viral mechanism of attacking a host. Accordingly, utilities of VGAM296 include diagnosis, prevention and treatment of viral infection by Vaccinia Virus. Specific functions, and accordingly utilities, of VGAM296 correlate with, and may be deduced from, the identity of the host target genes which VGAM296 binds and inhibits, and the function of these host target genes, as elaborated hereinbelow.

Nucleotide sequences of the VGAM296 precursor RNA, herein designated VGAM PRECURSOR RNA, and of the ‘diced’ VGAM296 RNA, herein designated VGAM RNA, and a schematic representation of the secondary folding of VGAM296 folded precursor RNA, herein designated VGAM FOLDED PRECURSOR RNA, of VGAM296 are further described hereinbelow with reference to Table 1.

Nucleotide sequences of host target binding sites, such as BINDING SITE-I, BINDING SITE-II and BINDING SITE-III of FIG. 1, found on VGAM296 host target RNA, and schematic representation of the complementarity of each of these host target binding sites to VGAM296 RNA, herein designated VGAM RNA, are described hereinbelow with reference to Table 2.

As mentioned hereinabove with reference to FIG. 1, a function of VGAM296 gene, herein designated VGAM is inhibition of expression of VGAM296 target genes. It is appreciated that specific functions, and accordingly utilities, of VGAM296 correlate with, and may be deduced from, the identity of the target genes which VGAM296 binds and inhibits, and the function of these target genes, as elaborated hereinbelow.

FLJ20139 (Accession NM_{—}017685) is a VGAM296 host target gene. FLJ20139 BINDING SITE is HOST TARGET binding site found in the 5′ untranslated region of mRNA encoded by FLJ20139, corresponding to a HOST TARGET binding site such as BINDING SITE I, BINDING SITE II or BINDING SITE III. Table 2 illustrates the complementarity of the nucleotide sequences of FLJ20139 BINDING SITE, designated SEQ ID: 1737, to the nucleotide sequence of VGAM296 RNA, herein designated VGAM RNA, also designated SEQ ID:631.

A function of VGAM296 is therefore inhibition of FLJ20139 (Accession NM_{—}017685). Accordingly, utilities of VGAM296 include diagnosis, prevention and treatment of diseases and clinical conditions associated with FLJ20139. LOC91796 (Accession XM_{—}040743) is another VGAM296 host target gene. LOC91796 BINDING SITE is HOST TARGET binding site found in the 3′ untranslated region of mRNA encoded by LOC91796, corresponding to a HOST TARGET binding site such as BINDING SITE I, BINDING SITE II or BINDING SITE III. Table 2 illustrates the complementarity of the nucleotide sequences of LOC91796 BINDING SITE, designated SEQ ID:2785, to the nucleotide sequence of VGAM296 RNA, herein designated VGAM RNA, also designated SEQ ID:631.

Another function of VGAM296 is therefore inhibition of LOC91796 (Accession XM_{—}040743). Accordingly, utilities of VGAM296 include diagnosis, prevention and treatment of diseases and clinical conditions associated with LOC91796. FIG. 1 further provides a conceptual description of a novel bioinformatically detected viral gene of the present invention, referred to here as Viral Genomic Address Messenger 297 (VGAM297) viral gene, which modulates expression of respective host target genes thereof, the function and utility of which host target genes is known in the art.

VGAM297 is a novel bioinformatically detected regulatory, non protein coding, viral micro RNA (miRNA) gene. The method by which VGAM297 was detected is described hereinabove with reference to FIGS. 1-8.

VGAM297 gene, herein designated VGAM GENE, is a viral gene contained in the genome of Vaccinia Virus. VGAM297 host target gene, herein designated VGAM HOST TARGET GENE, is a human gene contained in the human genome.

VGAM297 gene encodes a VGAM297 precursor RNA, herein designated VGAM PRECURSOR RNA. Similar to other miRNA genes, and unlike most ordinary genes, VGAM297 precursor RNA does not encode a protein. A nucleotide sequence identical or highly similar to the nucleotide sequence of VGAM297 precursor RNA is designated SEQ ID:283, and is provided hereinbelow with reference to the sequence listing part. Nucleotide sequence SEQ ID:283 is located at position 170506 relative to the genome of Vaccinia Virus.

VGAM297 precursor RNA folds onto itself, forming VGAM297 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM297 folded precursor RNA into VGAM297 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM297 RNA is designated SEQ ID:632, and is provided hereinbelow with reference to the sequence listing part.

VGAM297 host target gene, herein designated VGAM HOST TARGET GENE, encodes a corresponding messenger RNA, VGAM297 host target RNA, herein designated VGAM HOST TARGET RNA. VGAM297 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM297 RNA, herein designated VGAM RNA, binds complementarily to one or more host target binding sites located in untranslated regions of VGAM297 host target RNA, herein designated VGAM HOST TARGET RNA. This complementary binding is due to the fact that the nucleotide sequence of VGAM297 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM297 RNA, herein designated VGAM RNA, may have a different number of host target binding sites in untranslated regions of a VGAM297 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM297 RNA, herein designated VGAM RNA, to host target binding sites on VGAM297 host target RNA, herein designated VGAM HOST TARGET RNA, such as BINDING SITE I, BINDING SITE II and BINDING SITE III, inhibits translation of VGAM297 host target RNA into VGAM297 host target protein, herein designated VGAM HOST TARGET PROTEIN. VGAM host target protein is therefore outlined by a broken line.

It is appreciated that VGAM297 host target gene, herein designated VGAM HOST TARGET GENE, in fact represents a plurality of VGAM297 host target genes. The mRNA of each one of this plurality of VGAM297 host target genes comprises one or more host target binding sites, each having a nucleotide sequence which is at least partly complementary to VGAM297 RNA, herein designated VGAM RNA, and which when bound by VGAM297 RNA causes inhibition of translation of respective one or more VGAM297 host target proteins.

It is further appreciated by one skilled in the art that the mode of translational inhibition illustrated by FIG. 1 with specific reference to translational inhibition exerted by VGAM297 gene, herein designated VGAM GENE, on one or more VGAM297 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM297 is inhibition of expression of host target genes, as part of a novel viral mechanism of attacking a host. Accordingly, utilities of VGAM297 include diagnosis, prevention and treatment of viral infection by Vaccinia Virus. Specific functions, and accordingly utilities, of VGAM297 correlate with, and may be deduced from, the identity of the host target genes which VGAM297 binds and inhibits, and the function of these host target genes, as elaborated hereinbelow.

Nucleotide sequences of the VGAM297 precursor RNA, herein designated VGAM PRECURSOR RNA, and of the ‘diced’ VGAM297 RNA, herein designated VGAM RNA, and a schematic representation of the secondary folding of VGAM297 folded precursor RNA, herein designated VGAM FOLDED PRECURSOR RNA, of VGAM297 are further described hereinbelow with reference to Table 1.

Nucleotide sequences of host target binding sites, such as BINDING SITE-I, BINDING SITE-II and BINDING SITE-III of FIG. 1, found on VGAM297 host target RNA, and schematic representation of the complementarity of each of these host target binding sites to VGAM297 RNA, herein designated VGAM RNA, are described hereinbelow with reference to Table 2.

As mentioned hereinabove with reference to FIG. 1, a function of VGAM297 gene, herein designated VGAM is inhibition of expression of VGAM297 target genes. It is appreciated that specific functions, and accordingly utilities, of VGAM297 correlate with, and may be deduced from, the identity of the target genes which VGAM297 binds and inhibits, and the function of these target genes, as elaborated hereinbelow.

Cell Matrix Adhesion Regulator (CMAR, Accession NM_{—}005200) is a VGAM297 host target gene. CMAR BINDING SITE is HOST TARGET binding site found in the 3′ untranslated region of mRNA encoded by CMAR, corresponding to a HOST TARGET binding site such as BINDING SITE I, BINDING SITE II or BINDING SITE III. Table 2 illustrates the complementarity of the nucleotide sequences of CMAR BINDING SITE, designated SEQ ID:1194, to the nucleotide sequence of VGAM297 RNA, herein designated VGAM RNA, also designated SEQ ID:632.

A function of VGAM297 is therefore inhibition of Cell Matrix Adhesion Regulator (CMAR, Accession NM_{—}005200). Accordingly, utilities of VGAM297 include diagnosis, prevention and treatment of diseases and clinical conditions associated with CMAR. Corticotropin Releasing Hormone Receptor 1 (CRHR1, Accession NM_{—}004382) is another VGAM297 host target gene. CRHR1 BINDING SITE is HOST TARGET binding site found in the 3′ untranslated region of mRNA encoded by CRHR1, corresponding to a HOST TARGET binding site such as BINDING SITE I, BINDING SITE II or BINDING SITE III. Table 2 illustrates the complementarity of the nucleotide sequences of CRHR1 BINDING SITE, designated SEQ ID:1105, to the nucleotide sequence of VGAM297 RNA, herein designated VGAM RNA, also designated SEQ ID:632.

Another function of VGAM297 is therefore inhibition of Corticotropin Releasing Hormone Receptor 1 (CRHR1, Accession NM_{—}004382), a gene which likely mediates physiological and behavioral response to stress. Accordingly, utilities of VGAM297 include diagnosis, prevention and treatment of diseases and clinical conditions associated with CRHR1. The function of CRHR1 has been established by previous studies. Grammatopoulos et al. (1998) studied the expression of CRHR1 in human myometrium. They used RT-PCR, fluorescence in situ hybridization, and immunofluorescence to identify and localize the 4 subtypes, 1-alpha, 1-beta, 2-alpha, and the variant C, of CRHR1. The CRHR1 subtypes in myometrium exhibited differential expression patterns; in human pregnant myometrium at term, all 4 receptor subtypes were expressed, whereas only the 1-alpha and 1-beta receptor subtypes were found in the nonpregnant myometrium. The authors concluded that CRHR1 acting via different receptor subtypes is able to exert different actions on the myometrium in the pregnant state compared to the nonpregnant state. Furthermore, in the pregnant human uterus, receptors were localized in both smooth muscle and fibroblasts, suggesting that CRHR1 expression plays an important modulatory role in myometrial and possibly in cervical function Leproult et al. (2001) examined the effects of bright light on the profiles of hormones known to be affected by sleep deprivation (TSH; OMIM Ref. No. 188540) or involved in behavioral activation (cortisol). The early morning transition from dim to bright light suppressed melatonin secretion, induced an immediate, greater than 50% elevation of cortisol levels, and limited the deterioration of alertness normally associated with overnight sleep deprivation. No effect was detected on TSH profiles. The authors concluded that these data unambiguously demonstrate an effect of light on the corticotropic axis that is dependent on time of day Animal model experiments lend further support to the function of CRHR1. Sillaber et al. (2002) studied Crhr1 −/− mice generated by Timpl et al. (1998). In homozygous mutant mice, stress leads to enhanced and progressively increasing alcohol intake. The effect of repeated stress on alcohol drinking behavior appeared with a delay and persisted throughout life. It was associated with an up-regulation of the N-methyl-D-aspartate receptor subunit NR2B (OMIM Ref. No. 138252). Sillaber et al. (2002) concluded that alterations in the CRHR1 gene and adaptional changes in NR2B subunits may constitute a genetic risk factor for stress-induced alcohol drinking and alcoholism

It is appreciated that the abovementioned animal model for CRHR1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Leproult, R.; Colecchia, E. F.; L'Hermite-Baleriaux, M.; Van Cauter, E.: Transition from dim to bright light in the morning induces an immediate elevation of cortisol levels. J. Clin. Endocr. Metab. 86: 151-157, 2001.; and
Sillaber, I.; Rammes, G.; Zimmermann, S.; Mahal, B.; Zieglgansberger, W.; Wurst, W.; Holsboer, F.; Spanagel, R.: Enhanced and delayed stress-induced alcohol drinking in mice lacking fu.

Further studies establishing the function and utilities of CRHR1 are found in John Hopkins OMIM database record ID 122561, and in sited publications numbered 457-44 and 611-615 listed in the bibliography section hereinbelow, which are also hereby incorporated by reference. Diphtheria Toxin Receptor (heparin-binding epidermal growth factor-like growth factor) (DTR, Accession NM_{—}001945) is another VGAM297 host target gene. DTR BINDING SITE is HOST TARGET binding site found in the 3′ untranslated region of mRNA encoded by DTR, corresponding to a HOST TARGET binding site such as BINDING SITE I, BINDING SITE II or BINDING SITE III. Table 2 illustrates the complementarity of the nucleotide sequences of DTR BINDING SITE, designated SEQ ID:874, to the nucleotide sequence of VGAM297 RNA, herein designated VGAM RNA, also designated SEQ ID:632.

Another function of VGAM297 is therefore inhibition of Diphtheria Toxin Receptor (heparin-binding epidermal growth factor-like growth factor) (DTR, Accession NM_{—}001945), a gene which may be involved in macrophage-mediated cellular proliferation. Accordingly, utilities of VGAM297 include diagnosis, prevention and treatment of diseases and clinical conditions associated with DTR. The function of DTR has been established by previous studies. Mouse cells are naturally resistant to diphtheria toxin because they lack functional cell surface receptors for the toxin. Naglich et al. (1992) isolated a monkey cDNA encoding the diphtheria toxin sensitivity determinant by expression cloning in mouse L-M cells. Unlike wildtype L-M cells, transfected mouse cells were extremely toxin sensitive and specifically bound radioiodinated diphtheria toxin. Intoxication of the transfected cells required receptor-mediated endocytosis of the bound toxin. The cDNA was predicted to encode an integral membrane protein that is identical to the precursor of a heparin-binding EGF-like growth factor (7,8: Higashiyama et al., 1991, 1992). Thus the DT sensitivity protein is a growth factor precursor that the toxin exploits as a receptor. Fen et al. (1993) demonstrated that the heparin-binding EGF-like growth factor is encoded by a gene containing 6 exons and 5 intervening sequences spanning 14 kb of DNA. They demonstrated that treatment of endothelial cells with tumor necrosis factor-alpha (TNFA; 191160) produced a 10-fold increase in HB-EGF mRNA. By analysis of DNA isolated from human-mouse somatic hybrid cell lines, they assigned the gene to chromosome 5, thus confirming the assignment of the gene on the basis of its role in relation to diphtheria toxin susceptibility. The products of 5 distinct loci, EGF (OMIM Ref. No. 131530), TGFA (OMIM Ref. No. 190170), AREG (OMIM Ref. No. 104640), BCT (OMIM Ref. No. 600345), and HEGFL act as ligands for the epidermal growth factor receptor.

Full details of the abovementioned studies are described in the following publications, the disclosure of which are hereby incorporated by reference:
Fen, Z.; Dhadly, M. S.; Yoshizumi, M.; Hilkert, R. J.; Quertermous, T.; Eddy, R. L.; Shows, T. B.; Lee, M.-E.: Structural organization and chromosomal assignment of the gene encoding the human heparin-binding epidermal growth factor-like growth factor/diphtheria toxin receptor. Biochemistry 32: 7932-7938, 1993.; and
Naglich, J. G.; Metherall, J. E.; Russell, D. W.; Eidels, L.: Expression cloning of a diphtheria toxin receptor: identity with a heparin-binding EGF-like growth factor precursor. Cell.

Further studies establishing the function and utilities of DTR are found in John Hopkins OMIM database record ID 126150, and in sited publications numbered 950-95 and 52-57 listed in the bibliography section hereinbelow, which are also hereby incorporated by reference. Fibroblast Growth Factor 5 (FGF5, Accession NM_{—}004464) is another VGAM297 host target gene. FGF5 BINDING SITE1 and FGF5 BINDING SITE2 are HOST TARGET binding sites found in untranslated regions of mRNA encoded by FGF5, corresponding to HOST TARGET binding sites such as BINDING SITE I, BINDING SITE II or BINDING SITE III. Table 2 illustrates the complementarity of the nucleotide sequences of FGF5 BINDING SITE1 and FGF5 BINDING SITE2, designated SEQ ID:1117 and SEQ ID:2315 respectively, to the nucleotide sequence of VGAM297 RNA, herein designated VGAM RNA, also designated SEQ ID:632.

Another function of VGAM297 is therefore inhibition of Fibroblast Growth Factor 5 (FGF5, Accession NM_{—}004464), a gene which induces transformation and may regulate neuronal differentiation. Accordingly, utilities of VGAM297 include diagnosis, prevention and treatment of diseases and clinical conditions associated with FGF5. The function of FGF5 has been established by previous studies. Zhan et al. (1988) identified a fifth oncogene related to fibroblast growth factors and termed it FGF5. The other four are FGFA (OMIM Ref. No. 131220), FGFB (OMIM Ref. No. 134920), INT2 (OMIM Ref. No. 164950), and HST (OMIM Ref. No. 164980). FGF5 was discovered when it acquired transforming potential by a DNA rearrangement accompanying transfection of NIH 3T3 cells with human tumor DNA. Two regions of the FGF5 sequence, containing 122 of its 267 amino acid residues, were 40 to 50% homologous to the sequences of the 4 other members of the FGF oncogene family. FGF5, furthermore, was found to have a 3-exon structure typical for members of this family. FGF5 was found to be expressed in neonatal brain and in 3 of 13 human tumor cell lines examined. Nguyen et al. (1988) mapped FGF5 to 4q21 by in situ hybridization. Thus, it is not in the same cluster as the related HST and INT2 genes, which are coamplified in some tumor cells and were found by Nguyen et al. (1988), using pulsed field gel analysis, to be separated by only 40 kb. By polymerase chain reaction (PCR) amplification of target sequences in DNAs from somatic cell hybrids, Dionne et al. (1990) mapped the FGF5 gene to chromosome 4. By in situ chromosomal hybridization, Mattei et al. (1992) demonstrated that the corresponding gene in the mouse is on chromosome 5. Hebert et al. (1994) found that mice homozygous for a null allele of the Fgf5 gene, produced by gene targeting in embryonic stem cells, have abnormally long hair. This phenotype appeared identical to that of mice homozygous for the spontaneous mutation ‘angora’ (go). The transgenic mutant and the ‘go’ mutant failed to complement one another, and exon 1 of Fgf5 was found to be deleted in DNA from go homozygotes. Expression of Fgf5 is detected in hair follicles from wildtype mice and is localized to the outer root sheath during the anagen VI phase of the hair growth cycle. The findings were interpreted as evidence that FGF5 functions as an inhibitor of hair elongation, thus identifying a molecule whose normal function is apparently to regulate one step in the progression of the follicle through the hair growth cycle. It will be of interest to search for mutations in the FGF5 gene in hypertrichosis universalis (145700, 145701) as well as in other forms of hypertrichosis such as hairy elbows (OMIM Ref. No. 139600).

Full details of the abovementioned studies are described in the following publications, the disclosure of which are hereby incorporated by reference:
Zhan, X.; Bates, B.; Hu, X.; Goldfarb, M.: The human FGF5 oncogene encodes a novel protein related to fibroblast growth factors. Molec. Cell. Biol. 8: 3487-3495, 1988.; and
Hebert, J. M.; Rosenquist, T.; Gotz, J.; Martin, G. R.: FGF5 as a regulator of the hair growth cycle: evidence from targeted and spontaneous mutations. Cell 78: 1017-1025, 1994.

Further studies establishing the function and utilities of FGF5 are found in John Hopkins OMIM database record ID 165190, and in sited publications numbered 146-147, 4 and 148-149 listed in the bibliography section hereinbelow, which are also hereby incorporated by reference. Guanine Nucleotide Binding Protein-like 1 (GNL1, Accession XM_{—}166361) is another VGAM297 host target gene. GNL1 BINDING SITE1 and GNL1 BINDING SITE2 are HOST TARGET binding sites found in untranslated regions of mRNA encoded by GNL1, corresponding to HOST TARGET binding sites such as BINDING SITE I, BINDING SITE II or BINDING SITE III. Table 2 illustrates the complementarity of the nucleotide sequences of GNL1 BINDING SITE1 and GNL1 BINDING SITE2, designated SEQ ID:3550 and SEQ ID:3748 respectively, to the nucleotide sequence of VGAM297 RNA, herein designated VGAM RNA, also designated SEQ ID:632.

Another function of VGAM297 is therefore inhibition of Guanine Nucleotide Binding Protein-like 1 (GNL1, Accession XM_{—}166361). Accordingly, utilities of VGAM297 include diagnosis, prevention and treatment of diseases and clinical conditions associated with GNL1. Lactate Dehydrogenase B (LDHB, Accession NM_{—}002300) is another VGAM297 host target gene. LDHB BINDING SITE is HOST TARGET binding site found in the 5′ untranslated region of mRNA encoded by LDHB, corresponding to a HOST TARGET binding site such as BINDING SITE I, BINDING SITE II or BINDING SITE III. Table 2 illustrates the complementarity of the nucleotide sequences of LDHB BINDING SITE, designated SEQ ID:918, to the nucleotide sequence of VGAM297 RNA, herein designated VGAM RNA, also designated SEQ ID:632.

Another function of VGAM297 is therefore inhibition of Lactate Dehydrogenase B (LDHB, Accession NM_{—}002300), a gene which causes dehydrogenation of lactate. Accordingly, utilities of VGAM297 include diagnosis, prevention and treatment of diseases and clinical conditions associated with LDHB. The function of LDHB has been established by previous studies. LDHB and peptidase B (OMIM Ref. No. 169900) are linked (Santachiara et al., 1970) and both loci are on chromosome 12 (Chen et al., 1973). Kitamura et al. (1971) reported the first case of a complete deficiency of lactate dehydrogenase subunit H(B) in serum, saliva and erythrocytes of a 64-year-old male with mild diabetes. Study made on family members revealed low LDH activity in their serum also linked with decreased relative activity of the H4(B4) fraction. Based on the comparison of the calculated ratio of H to M subunits in normal and affected family members, it was hypothesized that the proband is homozygous while the abnormal family members are heterozygous, assuming a single gene is involved. Red cell metabolism in the proband was studied by Miwa et al. (1971); neither reticulocytosis nor hemolytic anemia was present. Thus, although LDHA deficiency leads to myoglobinuria and risk of renal failure after strenuous exercise, LDHB deficiency probably has no clear symptomatic consequences. As pointed out by Sudo (1993), LDH deficiency is of interest to laboratory medicine mainly because it can cause misdiagnosis in those disorders in which elevation of serum LDH is expected. LDH deficiency can probably be considered a ‘nondisease.’ In a screening of 2,880 blood samples from healthy persons in the Fukuoka Prefecture in Japan, Maekawa et al. (1994) found that the frequency of heterozygotes for either LDHA or LDHB deficiency was 0.104% at each locus. These estimated frequencies were slightly lower than, but not significantly different from, those found previously in the Shizuoka Prefecture. In a case of deletion of the short arm of chromosome 12, Weiss et al. (1973) found evidence that LDHB is located there. From study of somatic cell hybrids Hamerton et al. (1975) concluded that LDHB is in the 12q21-pter region. Rethore et al. (1975) found augmentation of LDHB activity in a boy trisomic for the short arm of chromosome 12. From study of 3 patients with different deletions of chromosome 12, Rethore et al. (1976) concluded that the G3PD locus is on the distal part of 12p, between p12.2 and 12pter, and that the LDHB locus is on the middle third between 12p12.1 and 12p12.2. The results for TPI were similar to those for G3PD, suggesting the same distal localization. Mohrenweiser and Neel (1981) identified thermolabile variants of lactate dehydrogenase B, glucosephosphate isomerase, and glucose-6-phosphate dehydrogenase. None was detectable as a variant by standard electrophoretic techniques. All were inherited. Steinbach and Rehder (1987) demonstrated dosage effect with LDHB in a case of tetrasomy of 12p. Sakai et al. (1987) isolated and sequenced LDHB cDNA. Nucleotide and amino acid sequences showed 68% and 75% identity, respectively, with those of LDHA. Sudo et al. (1990) demonstrated 93% homology between an LDHB processed pseudogene and the functional gene. The pseudogene was mapped to the X chromosome by dot blot analysis.

Full details of the abovementioned studies are described in the following publications, the disclosure of which are hereby incorporated by reference:
Kitamura, M.; Iijima, N.; Hashimoto, F.; Hiratsuka, A.: Hereditary deficiency of subunit H of lactate dehydrogenase. Clin. Chim. Acta 34: 419-423, 1971.; and
Sudo, K.; Maekawa, M.; Luedemann, M. M.; Deaven, L. L.; Li, S. S.-L.: Human lactate dehydrogenase-B processed pseudogene: nucleotide sequence analysis and assignment to the X-chromosome.

Further studies establishing the function and utilities of LDHB are found in John Hopkins OMIM database record ID 150100, and in sited publications numbered 1155, 2572-258 and 2592-2604 listed in the bibliography section hereinbelow, which are also hereby incorporated by reference. Latent Transforming Growth Factor Beta Binding Protein 2 (LTBP2, Accession NM_{—}000428) is another VGAM297 host target gene. LTBP2 BINDING SITE is HOST TARGET binding site found in the 3′ untranslated region of mRNA encoded by LTBP2, corresponding to a HOST TARGET binding site such as BINDING SITE I, BINDING SITE II or BINDING SITE III. Table 2 illustrates the complementarity of the nucleotide sequences of LTBP2 BINDING SITE, designated SEQ ID:740, to the nucleotide sequence of VGAM297 RNA, herein designated VGAM RNA, also designated SEQ ID:632.

Another function of VGAM297 is therefore inhibition of Latent Transforming Growth Factor Beta Binding Protein 2 (LTBP2, Accession NM_{—}000428), a gene which targets latent TGF-beta to the extracellular matrix. Accordingly, utilities of VGAM297 include diagnosis, prevention and treatment of diseases and clinical conditions associated with LTBP2. The function of LTBP2 has been established by previous studies. Transforming growth factors (TGFs) beta-1 (OMIM Ref. No. 190180), beta-2 (OMIM Ref. No. 190220), beta-3 (OMIM Ref. No. 190230), and others have both stimulatory and inhibitory effects on the growth of different cell types and play a role in the production and degradation of the extracellular matrix. TGF-beta molecules are secreted in the form of latent large molecular mass complexes that contain other proteins, such as latent TGF-beta-1 binding protein (LTBP1; 150390). There is evidence that these binding proteins modulate TGF-beta bioavailability Animal model experiments lend further support to the function of LTBP2. Dabovic et al. (2002) created an Ltbp3-null mutation in the mouse by gene targeting. Mice homozygous for the mutation developed craniofacial malformations by day 10. At 2 months, there was a pronounced rounding of the cranial vault, extension of the mandible beyond the maxilla, and kyphosis. Between 6 and 9 months of age, mutant mice also developed osteosclerosis and osteoarthritis. The pathologic changes were consistent with perturbed TGF-beta signaling in the skull and long bones

It is appreciated that the abovementioned animal model for LTBP2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Oklu, R.; Hesketh, R.: The latent transforming growth factor beta binding protein (LTBP) family. Biochem. J. 352: 601-610, 2000.; and
Dabovic, B.; Chen, Y.; Colarossi, C.; Obata, H.; Zambuto, L.; Perle, M. A.; Rifkin, D. B.: Bone abnormalities in latent TGF-beta binding protein (Ltbp)-3-null mice indicate a role for Ltb.

Further studies establishing the function and utilities of LTBP2 are found in John Hopkins OMIM database record ID 602090, and in sited publications numbered 1478-147 and 3009-1481 listed in the bibliography section hereinbelow, which are also hereby incorporated by reference. MAP/microtubule Affinity-regulating Kinase 1 (MARK1, Accession NM_{—}018650) is another VGAM297 host target gene. MARK1 BINDING SITE is HOST TARGET binding site found in the 5′ untranslated region of mRNA encoded by MARK1, corresponding to a HOST TARGET binding site such as BINDING SITE I, BINDING SITE II or BINDING SITE III. Table 2 illustrates the complementarity of the nucleotide sequences of MARK1 BINDING SITE, designated SEQ ID: 1856, to the nucleotide sequence of VGAM297 RNA, herein designated VGAM RNA, also designated SEQ ID:632.

Another function of VGAM297 is therefore inhibition of MAP/microtubule Affinity-regulating Kinase 1 (MARK1, Accession NM_{—}018650), a gene which phosphorylate microtubule-associated proteins and trigger microtubule disruption. Accordingly, utilities of VGAM297 include diagnosis, prevention and treatment of diseases and clinical conditions associated with MARK1. The function of MARK1 and its association with various diseases and clinical conditions, has been established by previous studies, as described hereinabove with reference to VGAM38. Plasminogen Activator, Tissue (PLAT, Accession NM_{—}033011) is another VGAM297 host target gene. PLAT BINDING SITE1 and PLAT BINDING SITE2 are HOST TARGET binding sites found in untranslated regions of mRNA encoded by PLAT, corresponding to HOST TARGET binding sites such as BINDING SITE I, BINDING SITE II or BINDING SITE III. Table 2 illustrates the complementarity of the nucleotide sequences of PLAT BINDING SITE1 and PLAT BINDING SITE2, designated SEQ ID:2306 and SEQ ID:788 respectively, to the nucleotide sequence of VGAM297 RNA, herein designated VGAM RNA, also designated SEQ ID:632.

Another function of VGAM297 is therefore inhibition of Plasminogen Activator, Tissue (PLAT, Accession NM_{—}033011). Accordingly, utilities of VGAM297 include diagnosis, prevention and treatment of diseases and clinical conditions associated with PLAT. Paxillin (PXN, Accession NM_{—}002859) is another VGAM297 host target gene. PXN BINDING SITE is HOST TARGET binding site found in the 3′ untranslated region of mRNA encoded by PXN, corresponding to a HOST TARGET binding site such as BINDING SITE I, BINDING SITE II or BINDING SITE III. Table 2 illustrates the complementarity of the nucleotide sequences of PXN BINDING SITE, designated SEQ ID:965, to the nucleotide sequence of VGAM297 RNA, herein designated VGAM RNA, also designated SEQ ID:632.

Another function of VGAM297 is therefore inhibition of Paxillin (PXN, Accession NM_{—}002859), a gene which interacts with multiple structural and signalling proteins. Accordingly, utilities of VGAM297 include diagnosis, prevention and treatment of diseases and clinical conditions associated with PXN. The function of PXN has been established by previous studies. Glenney and Zokas (1989) used an antiphosphotyrosine antibody to identify proteins that are phosphorylated in Rous sarcoma virus-transformed chick embryo fibroblasts, and found a 76-kD protein that localizes to focal adhesions at the ends of actin-containing stress fibers in nontransformed cells. Turner et al. (1990) purified this protein from chicken gizzard smooth muscle, and named it paxillin (‘paxillus’ means ‘small stake’ or ‘peg’ in Latin) as a protein tethered to the membrane at focal adhesions. Paxillin migrates as a diffuse 65- to 70-kD band on SDS-PAGE. Salgia et al. (1995) stated that transmembrane integrin molecules (see OMIM Ref. No. 600536) connect the actin cytoskeleton to the extracellular matrix within focal adhesions. They cloned human paxillin by screening an expression library with antipaxillin antibody. The predicted 557-amino acid protein has a predicted molecular mass of 61 kD, but an observed molecular mass of 68 kD, suggesting that it is either posttranslationally modified or migrates aberrantly due to high (10%) proline content. The paxillin protein contains 4 LIM domains, a proline-rich domain containing a consensus SH3-binding site, and 3 potential SH2-binding sites. On Northern blots, paxillin was expressed as a 3.7-kb mRNA in all tissues tested. Mazaki et al. (1997) found that the paxillin gene can be alternatively spliced to include 1 of 2 alternative exons, generating beta and gamma isoforms. As observed on Northern blots, Western blots, and with RT-PCR, the beta and gamma isoforms are expressed only in certain cancer cell lines. The different isoforms had different affinities for cellular proteins, including vinculin (OMIM Ref. No. 193065) and FAK (OMIM Ref. No. 600758), suggesting that each acts as a distinct module involved in different functions of integrins.

Full details of the abovementioned studies are described in the following publications, the disclosure of which are hereby incorporated by reference:
Salgia, R.; Li, J.-L.; Lo, S. H.; Brunkhorst, B.; Kansas, G. S.; Sobhany, E. S.; Sun, Y.; Pisick, E.; Hallek, M.; Ernst, T.; Tantravahi, R.; Chen, L. B.; Griffin, J. D.: Molecular cloning of human paxillin, a focal adhesion protein phosphorylated by P210(BCR/ABL). J. Biol. Chem. 270: 5039-5047, 1995.; and
Mazaki, Y.; Hashimoto, S.; Sabe, H.: Monocyte cells and cancer cells express novel paxillin isoforms with different binding properties to focal adhesion proteins. J. Biol. Chem. 272: 74.

Further studies establishing the function and utilities of PXN are found in John Hopkins OMIM database record ID 602505, and in sited publications numbered 2020-2024 listed in the bibliography section hereinbelow, which are also hereby incorporated by reference. Ribonuclease/angiogenin Inhibitor (RNH, Accession XM_{—}006139) is another VGAM297 host target gene. RNH BINDING SITE is HOST TARGET binding site found in the 5′ untranslated region of mRNA encoded by RNH, corresponding to a HOST TARGET binding site such as BINDING SITE I, BINDING SITE II or BINDING SITE III. Table 2 illustrates the complementarity of the nucleotide sequences of RNH BINDING SITE, designated SEQ ID:2536, to the nucleotide sequence of VGAM297 RNA, herein designated VGAM RNA, also designated SEQ ID:632.

Another function of VGAM297 is therefore inhibition of Ribonuclease/angiogenin Inhibitor (RNH, Accession XM_{—}006139), a gene which is an inhibitor of pancreatic rnase and angiogenin. may also function in the modulation of cellular activities. Accordingly, utilities of VGAM297 include diagnosis, prevention and treatment of diseases and clinical conditions associated with RNH. The function of RNH has been established by previous studies. Placental ribonuclease inhibitor is a member of a family of proteinaceous cytoplasmic RNase inhibitors that occur in many tissues and bind to both intracellular and extracellular RNases. In addition to control of intracellular RNases, the inhibitor may have a role in the regulation of angiogenin (OMIM Ref. No. 105850). Ribonuclease inhibitor, of 50,000 Da, binds to ribonucleases and holds them in a latent form. Since neutral and alkaline ribonucleases probably play a critical role in the turnover of RNA in eukaryotic cells, RNH may be essential for control of mRNA turnover; the interaction of eukaryotic cells with ribonuclease may be reversible in vivo. Lee et al. (1988) determined the primary structure of PRI from the cDNA. The mature protein encodes a 460-amino acid polypeptide with a molecular mass of 49,847 kD. The amino acid sequence contains 7 direct internal repeat units, each 57 amino acids in length. These repeat units comprise 87% of the molecule. The average degree of identity between any 2 is 39%. By study of human-rodent somatic cell hybrids and by in situ hybridization, Weremowicz et al. (1990) mapped the PRI gene to 11p15. The localization was further refined to 11p15.5, distal to the IGF2 gene, by in situ hybridization to metaphase chromosomes from a cell line with a well-characterized translocation involving a breakpoint between IGF2 (OMIM Ref. No. 147470) and HRAS (OMIM Ref. No. 190020). Zneimer et al. (1990) localized the RNH gene to 11p15.5 by in situ hybridization

Full details of the abovementioned studies are described in the following publications, the disclosure of which are hereby incorporated by reference:
Weremowicz, S.; Fox, E. A.; Morton, C. C.; Vallee, B. L.: The placental ribonuclease inhibitor (RNH) gene is located on chromosome subband 11p15.5. Genomics 8: 717-721, 1990.; and
Zneimer, S. M.; Crawford, D.; Schneider, N. R.; Beutler, B.: Mapping of the human ribonuclease inhibitor gene (RNH) to chromosome 11p15 by in situ hybridization. Genomics 8: 175-178, 19.

Further studies establishing the function and utilities of RNH are found in John Hopkins OMIM database record ID 173320, and in sited publications numbered 2209-2211 listed in the bibliography section hereinbelow, which are also hereby incorporated by reference. Sodium Channel, Voltage-gated, Type IV, Alpha Polypeptide (SCN4A, Accession NM_{—}000334) is another VGAM297 host target gene. SCN4A BINDING SITE is HOST TARGET binding site found in the 3′ untranslated region of mRNA encoded by SCN4A, corresponding to a HOST TARGET binding site such as BINDING SITE I, BINDING SITE II or BINDING SITE III. Table 2 illustrates the complementarity of the nucleotide sequences of SCN4A BINDING SITE, designated SEQ ID:730, to the nucleotide sequence of VGAM297 RNA, herein designated VGAM RNA, also designated SEQ ID:632.

Another function of VGAM297 is therefore inhibition of Sodium Channel, Voltage-gated, Type IV, Alpha Polypeptide (SCN4A, Accession NM_{—}000334). Accordingly, utilities of VGAM297 include diagnosis, prevention and treatment of diseases and clinical conditions associated with SCN4A. SH3-domain GRB2-like 1 (SH3GL1, Accession NM_{—}003025) is another VGAM297 host target gene. SH3GL1 BINDING SITE is HOST TARGET binding site found in the 3′ untranslated region of mRNA encoded by SH3GL1, corresponding to a HOST TARGET binding site such as BINDING SITE I, BINDING SITE II or BINDING SITE III. Table 2 illustrates the complementarity of the nucleotide sequences of SH3GL1 BINDING SITE, designated SEQ ID:982, to the nucleotide sequence of VGAM297 RNA, herein designated VGAM RNA, also designated SEQ ID:632.

Another function of VGAM297 is therefore inhibition of SH3-domain GRB2-like 1 (SH3GL1, Accession NM_{—}003025). Accordingly, utilities of VGAM297 include diagnosis, prevention and treatment of diseases and clinical conditions associated with SH3GL1. Zinc Finger Protein 261 (ZNF261, Accession NM_{—}005096) is another VGAM297 host target gene. ZNF261 BINDING SITE is HOST TARGET binding site found in the 3′ untranslated region of mRNA encoded by ZNF261, corresponding to a HOST TARGET binding site such as BINDING SITE I, BINDING SITE II or BINDING SITE III. Table 2 illustrates the complementarity of the nucleotide sequences of ZNF261 BINDING SITE, designated SEQ ID:1184, to the nucleotide sequence of VGAM297 RNA, herein designated VGAM RNA, also designated SEQ ID:632.

Another function of VGAM297 is therefore inhibition of Zinc Finger Protein 261 (ZNF261, Accession NM_{—}005096). Accordingly, utilities of VGAM297 include diagnosis, prevention and treatment of diseases and clinical conditions associated with ZNF261. A Kinase (PRKA) Anchor Protein 3 (AKAP3, Accession NM_{—}006422) is another VGAM297 host target gene. AKAP3 BINDING SITE is HOST TARGET binding site found in the 3′ untranslated region of mRNA encoded by AKAP3, corresponding to a HOST TARGET binding site such as BINDING SITE I, BINDING SITE II or BINDING SITE III. Table 2 illustrates the complementarity of the nucleotide sequences of AKAP3 BINDING SITE, designated SEQ ID:1300, to the nucleotide sequence of VGAM297 RNA, herein designated VGAM RNA, also designated SEQ ID:632.

Another function of VGAM297 is therefore inhibition of A Kinase (PRKA) Anchor Protein 3 (AKAP3, Accession NM_{—}006422). Accordingly, utilities of VGAM297 include diagnosis, prevention and treatment of diseases and clinical conditions associated with AKAP3. C3F (Accession NM_{—}005768) is another VGAM297 host target gene. C3F BINDING SITE is HOST TARGET binding site found in the 3′ untranslated region of mRNA encoded by C3F, corresponding to a HOST TARGET binding site such as BINDING SITE I, BINDING SITE II or BINDING SITE III. Table 2 illustrates the complementarity of the nucleotide sequences of C3F BINDING SITE, designated SEQ ID:1246, to the nucleotide sequence of VGAM297 RNA, herein designated VGAM RNA, also designated SEQ ID:632.

Another function of VGAM297 is therefore inhibition of C3F (Accession NM_{—}005768). Accordingly, utilities of VGAM297 include diagnosis, prevention and treatment of diseases and clinical conditions associated with C3F. CL24751 (Accession XM_{—}035634) is another VGAM297 host target gene. CL24751 BINDING SITE is HOST TARGET binding site found in the 5′ untranslated region of mRNA encoded by CL24751, corresponding to a HOST TARGET binding site such as BINDING SITE I, BINDING SITE II or BINDING SITE III. Table 2 illustrates the complementarity of the nucleotide sequences of CL24751 BINDING SITE, designated SEQ ID:2698, to the nucleotide sequence of VGAM297 RNA, herein designated VGAM RNA, also designated SEQ ID:632.

Another function of VGAM297 is therefore inhibition of CL24751 (Accession XM_{—}035634). Accordingly, utilities of VGAM297 include diagnosis, prevention and treatment of diseases and clinical conditions associated with CL24751. DIS3 (Accession NM_{—}014953) is another VGAM297 host target gene. DIS3 BINDING SITE is HOST TARGET binding site found in the 3′ untranslated region of mRNA encoded by DIS3, corresponding to a HOST TARGET binding site such as BINDING SITE I, BINDING SITE II or BINDING SITE III. Table 2 illustrates the complementarity of the nucleotide sequences of DIS3 BINDING SITE, designated SEQ ID:1597, to the nucleotide sequence of VGAM297 RNA, herein designated VGAM RNA, also designated SEQ ID:632.

Another function of VGAM297 is therefore inhibition of DIS3 (Accession NM_{—}014953). Accordingly, utilities of VGAM297 include diagnosis, prevention and treatment of diseases and clinical conditions associated with DIS3. FLJ10700 (Accession NM_{—}018182) is another VGAM297 host target gene. FLJ10700 BINDING SITE is HOST TARGET binding site found in the 3′ untranslated region of mRNA encoded by FLJ10700, corresponding to a HOST TARGET binding site such as BINDING SITE I, BINDING SITE II or BINDING SITE III. Table 2 illustrates the complementarity of the nucleotide sequences of FLJ10700 BINDING SITE, designated SEQ ID:1794, to the nucleotide sequence of VGAM297 RNA, herein designated VGAM RNA, also designated SEQ ID:632.

Another function of VGAM297 is therefore inhibition of FLJ10700 (Accession NM_{—}018182). Accordingly, utilities of VGAM297 include diagnosis, prevention and treatment of diseases and clinical conditions associated with FLJ10700. FLJ13102 (Accession NM_{—}024887) is another VGAM297 host target gene. FLJ13102 BINDING SITE is HOST TARGET binding site found in the 3′ untranslated region of mRNA encoded by FLJ13102, corresponding to a HOST TARGET binding site such as BINDING SITE I, BINDING SITE II or BINDING SITE III. Table 2 illustrates the complementarity of the nucleotide sequences of FLJ13102 BINDING SITE, designated SEQ ID:2104, to the nucleotide sequence of VGAM297 RNA, herein designated VGAM RNA, also designated SEQ ID:632.

Another function of VGAM297 is therefore inhibition of FLJ13102 (Accession NM_{—}024887). Accordingly, utilities of VGAM297 include diagnosis, prevention and treatment of diseases and clinical conditions associated with FLJ13102. FLJ32978 (Accession NM_{—}144625) is another VGAM297 host target gene. FLJ32978 BINDING SITE is HOST TARGET binding site found in the 3′ untranslated region of mRNA encoded by FLJ32978, corresponding to a HOST TARGET binding site such as BINDING SITE I, BINDING SITE II or BINDING SITE III. Table 2 illustrates the complementarity of the nucleotide sequences of FLJ32978 BINDING SITE, designated SEQ ID:2488, to the nucleotide sequence of VGAM297 RNA, herein designated VGAM RNA, also designated SEQ ID:632.

Another function of VGAM297 is therefore inhibition of FLJ32978 (Accession NM_{—}144625). Accordingly, utilities of VGAM297 include diagnosis, prevention and treatment of diseases and clinical conditions associated with FLJ32978. Heat Shock 27 kDa Protein Family, Member 7 (cardiovascular) (HSPB7, Accession NM_{—}014424) is another VGAM297 host target gene. HSPB7 BINDING SITE is HOST TARGET binding site found in the 3′ untranslated region of mRNA encoded by HSPB7, corresponding to a HOST TARGET binding site such as BINDING SITE I, BINDING SITE II or BINDING SITE III. Table 2 illustrates the complementarity of the nucleotide sequences of HSPB7 BINDING SITE, designated SEQ ID: 1499, to the nucleotide sequence of VGAM297 RNA, herein designated VGAM RNA, also designated SEQ ID:632.

Another function of VGAM297 is therefore inhibition of Heat Shock 27 kDa Protein Family, Member 7 (cardiovascular) (HSPB7, Accession NM_{—}014424). Accordingly, utilities of VGAM297 include diagnosis, prevention and treatment of diseases and clinical conditions associated with HSPB7. HTCD37 (Accession XM_{—}041884) is another VGAM297 host target gene. HTCD37 BINDING SITE is HOST TARGET binding site found in the 3′ untranslated region of mRNA encoded by HTCD37, corresponding to a HOST TARGET binding site such as BINDING SITE I, BINDING SITE II or BINDING SITE III. Table 2 illustrates the complementarity of the nucleotide sequences of HTCD37 BINDING SITE, designated SEQ ID:2796, to the nucleotide sequence of VGAM297 RNA, herein designated VGAM RNA, also designated SEQ ID:632.

Another function of VGAM297 is therefore inhibition of HTCD37 (Accession XM_{—}041884). Accordingly, utilities of VGAM297 include diagnosis, prevention and treatment of diseases and clinical conditions associated with HTCD37. KIAA0280 (Accession XM_{—}166238) is another VGAM297 host target gene. KIAA0280 BINDING SITE is HOST TARGET binding site found in the 3′ untranslated region of mRNA encoded by KIAA0280, corresponding to a HOST TARGET binding site such as BINDING SITE I, BINDING SITE II or BINDING SITE III. Table 2 illustrates the complementarity of the nucleotide sequences of KIAA0280 BINDING SITE, designated SEQ ID:3537, to the nucleotide sequence of VGAM297 RNA, herein designated VGAM RNA, also designated SEQ ID:632.

Another function of VGAM297 is therefore inhibition of KIAA0280 (Accession XM_{—}166238). Accordingly, utilities of VGAM297 include diagnosis, prevention and treatment of diseases and clinical conditions associated with KIAA0280. KIAA0450 (Accession NM_{—}014638) is another VGAM297 host target gene. KIAA0450 BINDING SITE is HOST TARGET binding site found in the 5′ untranslated region of mRNA encoded by KIAA0450, corresponding to a HOST TARGET binding site such as BINDING SITE I, BINDING SITE II or BINDING SITE III. Table 2 illustrates the complementarity of the nucleotide sequences of KIAA0450 BINDING SITE, designated SEQ ID:1511, to the nucleotide sequence of VGAM297 RNA, herein designated VGAM RNA, also designated SEQ ID:632.

Another function of VGAM297 is therefore inhibition of KIAA0450 (Accession NM_{—}014638). Accordingly, utilities of VGAM297 include diagnosis, prevention and treatment of diseases and clinical conditions associated with KIAA0450. KIAA0731 (Accession XM_{—}039975) is another VGAM297 host target gene. KIAA0731 BINDING SITE is HOST TARGET binding site found in the 3′ untranslated region of mRNA encoded by KIAA0731, corresponding to a HOST TARGET binding site such as BINDING SITE I, BINDING SITE II or BINDING SITE III. Table 2 illustrates the complementarity of the nucleotide sequences of KIAA0731 BINDING SITE, designated SEQ ID:2767, to the nucleotide sequence of VGAM297 RNA, herein designated VGAM RNA, also designated SEQ ID:632.

Another function of VGAM297 is therefore inhibition of KIAA0731 (Accession XM_{—}039975). Accordingly, utilities of VGAM297 include diagnosis, prevention and treatment of diseases and clinical conditions associated with KIAA0731. KIAA1462 (Accession XM_{—}166132) is another VGAM297 host target gene. KIAA1462 BINDING SITE is HOST TARGET binding site found in the 3′ untranslated region of mRNA encoded by KIAA1462, corresponding to a HOST TARGET binding site such as BINDING SITE I, BINDING SITE II or BINDING SITE III. Table 2 illustrates the complementarity of the nucleotide sequences of KIAA1462 BINDING SITE, designated SEQ ID:3523, to the nucleotide sequence of VGAM297 RNA, herein designated VGAM RNA, also designated SEQ ID:632.

Another function of VGAM297 is therefore inhibition of KIAA1462 (Accession XM_{—}166132). Accordingly, utilities of VGAM297 include diagnosis, prevention and treatment of diseases and clinical conditions associated with KIAA1462. LIM Domain Kinase 2 (LIMK2, Accession NM_{—}016733) is another VGAM297 host target gene. LIMK2 BINDING SITE1 and LIMK2 BINDING SITE2 are HOST TARGET binding sites found in untranslated regions of mRNA encoded by LIMK2, corresponding to HOST TARGET binding sites such as BINDING SITE I, BINDING SITE II or BINDING SITE III. Table 2 illustrates the complementarity of the nucleotide sequences of LIMK2 BINDING SITE1 and LIMK2 BINDING SITE2, designated SEQ ID:1710 and SEQ ID:1225 respectively, to the nucleotide sequence of VGAM297 RNA, herein designated VGAM RNA, also designated SEQ ID:632.

Another function of VGAM297 is therefore inhibition of LIM Domain Kinase 2 (LIMK2, Accession NM_{—}016733). Accordingly, utilities of VGAM297 include diagnosis, prevention and treatment of diseases and clinical conditions associated with LIMK2. MGC3101 (Accession NM_{—}024043) is another VGAM297 host target gene. MGC3101 BINDING SITE is HOST TARGET binding site found in the 3′ untranslated region of mRNA encoded by MGC3101, corresponding to a HOST TARGET binding site such as BINDING SITE I, BINDING SITE II or BINDING SITE III. Table 2 illustrates the complementarity of the nucleotide sequences of MGC3101 BINDING SITE, designated SEQ ID:2046, to the nucleotide sequence of VGAM297 RNA, herein designated VGAM RNA, also designated SEQ ID:632.

Another function of VGAM297 is therefore inhibition of MGC3101 (Accession NM_{—}024043). Accordingly, utilities of VGAM297 include diagnosis, prevention and treatment of diseases and clinical conditions associated with MGC3101. Nuclear Receptor Coactivator 2 (NCOA2, Accession NM_{—}006540) is another VGAM297 host target gene. NCOA2 BINDING SITE is HOST TARGET binding site found in the 3′ untranslated region of mRNA encoded by NCOA2, corresponding to a HOST TARGET binding site such as BINDING SITE I, BINDING SITE II or BINDING SITE III. Table 2 illustrates the complementarity of the nucleotide sequences of NCOA2 BINDING SITE, designated SEQ ID:1307, to the nucleotide sequence of VGAM297 RNA, herein designated VGAM RNA, also designated SEQ ID:632.

Another function of VGAM297 is therefore inhibition of Nuclear Receptor Coactivator 2 (NCOA2, Accession NM_{—}006540). Accordingly, utilities of VGAM297 include diagnosis, prevention and treatment of diseases and clinical conditions associated with NCOA2. RNA Binding Motif Protein 14 (RBM14, Accession NM_{—}006328) is another VGAM297 host target gene. RBM14 BINDING SITE is HOST TARGET binding site found in the 3′ untranslated region of mRNA encoded by RBM14, corresponding to a HOST TARGET binding site such as BINDING SITE I, BINDING SITE II or BINDING SITE III. Table 2 illustrates the complementarity of the nucleotide sequences of RBM14 BINDING SITE, designated SEQ ID:1289, to the nucleotide sequence of VGAM297 RNA, herein designated VGAM RNA, also designated SEQ ID:632.

Another function of VGAM297 is therefore inhibition of RNA Binding Motif Protein 14 (RBM14, Accession NM_{—}006328). Accordingly, utilities of VGAM297 include diagnosis, prevention and treatment of diseases and clinical conditions associated with RBM14. SCYA5 (Accession NM_{—}002985) is another VGAM297 host target gene. SCYA5 BINDING SITE is HOST TARGET binding site found in the 3′ untranslated region of mRNA encoded by SCYA5, corresponding to a HOST TARGET binding site such as BINDING SITE I, BINDING SITE II or BINDING SITE III. Table 2 illustrates the complementarity of the nucleotide sequences of SCYA5 BINDING SITE, designated SEQ ID:979, to the nucleotide sequence of VGAM297 RNA, herein designated VGAM RNA, also designated SEQ ID:632.

Another function of VGAM297 is therefore inhibition of SCYA5 (Accession NM_{—}002985). Accordingly, utilities of VGAM297 include diagnosis, prevention and treatment of diseases and clinical conditions associated with SCYA5. U5-116KD (Accession NM_{—}004247) is another VGAM297 host target gene. U5-116KD BINDING SITE is HOST TARGET binding site found in the 3′ untranslated region of mRNA encoded by U5-116KD, corresponding to a HOST TARGET binding site such as BINDING SITE I, BINDING SITE II or BINDING SITE III. Table 2 illustrates the complementarity of the nucleotide sequences of U5-116KD BINDING SITE, designated SEQ ID: 1095, to the nucleotide sequence of VGAM297 RNA, herein designated VGAM RNA, also designated SEQ ID:632.

Another function of VGAM297 is therefore inhibition of U5-116KD (Accession NM_{—}004247). Accordingly, utilities of VGAM297 include diagnosis, prevention and treatment of diseases and clinical conditions associated with U5-116KD. WSB1 (Accession NM_{—}134264) is another VGAM297 host target gene. WSB1 BINDING SITE is HOST TARGET binding site found in the 3′ untranslated region of mRNA encoded by WSB1, corresponding to a HOST TARGET binding site such as BINDING SITE I, BINDING SITE II or BINDING SITE III. Table 2 illustrates the complementarity of the nucleotide sequences of WSB1 BINDING SITE, designated SEQ ID:2429, to the nucleotide sequence of VGAM297 RNA, herein designated VGAM RNA, also designated SEQ ID:632.

Another function of VGAM297 is therefore inhibition of WSB1 (Accession NM_{—}134264). Accordingly, utilities of VGAM297 include diagnosis, prevention and treatment of diseases and clinical conditions associated with WSB1. LOC144501 (Accession XM_{—}096612) is another VGAM297 host target gene. LOC144501 BINDING SITE is HOST TARGET binding site found in the 3′ untranslated region of mRNA encoded by LOC144501, corresponding to a HOST TARGET binding site such as BINDING SITE I, BINDING SITE II or BINDING SITE III. Table 2 illustrates the complementarity of the nucleotide sequences of LOC144501 BINDING SITE, designated SEQ ID:3268, to the nucleotide sequence of VGAM297 RNA, herein designated VGAM RNA, also designated SEQ ID:632.

Another function of VGAM297 is therefore inhibition of LOC144501 (Accession XM_{—}096612). Accordingly, utilities of VGAM297 include diagnosis, prevention and treatment of diseases and clinical conditions associated with LOC144501. LOC146940 (Accession XM_{—}085648) is another VGAM297 host target gene. LOC146940 BINDING SITE is HOST TARGET binding site found in the 3′ untranslated region of mRNA encoded by LOC146940, corresponding to a HOST TARGET binding site such as BINDING SITE I, BINDING SITE II or BINDING SITE III. Table 2 illustrates the complementarity of the nucleotide sequences of LOC146940 BINDING SITE, designated SEQ ID:3101, to the nucleotide sequence of VGAM297 RNA, herein designated VGAM RNA, also designated SEQ ID:632.

Another function of VGAM297 is therefore inhibition of LOC146940 (Accession XM_{—}085648). Accordingly, utilities of VGAM297 include diagnosis, prevention and treatment of diseases and clinical conditions associated with LOC146940. LOC197003 (Accession XM_{—}113798) is another VGAM297 host target gene. LOC197003 BINDING SITE is HOST TARGET binding site found in the 3′ untranslated region of mRNA encoded by LOC197003, corresponding to a HOST TARGET binding site such as BINDING SITE I, BINDING SITE II or BINDING SITE III. Table 2 illustrates the complementarity of the nucleotide sequences of LOC197003 BINDING SITE, designated SEQ ID:3418, to the nucleotide sequence of VGAM297 RNA, herein designated VGAM RNA, also designated SEQ ID:632.

Another function of VGAM297 is therefore inhibition of LOC197003 (Accession XM_{—}113798). Accordingly, utilities of VGAM297 include diagnosis, prevention and treatment of diseases and clinical conditions associated with LOC197003. LOC200953 (Accession XM_{—}117302) is another VGAM297 host target gene. LOC200953 BINDING SITE is HOST TARGET binding site found in the 5′ untranslated region of mRNA encoded by LOC200953, corresponding to a HOST TARGET binding site such as BINDING SITE I, BINDING SITE II or BINDING SITE III. Table 2 illustrates the complementarity of the nucleotide sequences of LOC200953 BINDING SITE, designated SEQ ID:3482, to the nucleotide sequence of VGAM297 RNA, herein designated VGAM RNA, also designated SEQ ID:632.

Another function of VGAM297 is therefore inhibition of LOC200953 (Accession XM_{—}117302). Accordingly, utilities of VGAM297 include diagnosis, prevention and treatment of diseases and clinical conditions associated with LOC200953. LOC257541 (Accession XM_{—}175175) is another VGAM297 host target gene. LOC257541 BINDING SITE is HOST TARGET binding site found in the 3′ untranslated region of mRNA encoded by LOC257541, corresponding to a HOST TARGET binding site such as BINDING SITE I, BINDING SITE II or BINDING SITE III. Table 2 illustrates the complementarity of the nucleotide sequences of LOC257541 BINDING SITE, designated SEQ ID:3746, to the nucleotide sequence of VGAM297 RNA, herein designated VGAM RNA, also designated SEQ ID:632.

Another function of VGAM297 is therefore inhibition of LOC257541 (Accession XM_{—}175175). Accordingly, utilities of VGAM297 include diagnosis, prevention and treatment of diseases and clinical conditions associated with LOC257541. FIG. 1 further provides a conceptual description of a novel bioinformatically detected viral gene of the present invention, referred to here as Viral Genomic Address Messenger 298 (VGAM298) viral gene, which modulates expression of respective host target genes thereof, the function and utility of which host target genes is known in the art.

VGAM298 is a novel bioinformatically detected regulatory, non protein coding, viral micro RNA (miRNA) gene. The method by which VGAM298 was detected is described hereinabove with reference to FIGS. 1-8.

VGAM298 gene, herein designated VGAM GENE, is a viral gene contained in the genome of Vaccinia Virus. VGAM298 host target gene, herein designated VGAM HOST TARGET GENE, is a human gene contained in the human genome.

VGAM298 gene encodes a VGAM298 precursor RNA, herein designated VGAM PRECURSOR RNA. Similar to other miRNA genes, and unlike most ordinary genes, VGAM298 precursor RNA does not encode a protein. A nucleotide sequence identical or highly similar to the nucleotide sequence of VGAM298 precursor RNA is designated SEQ ID:284, and is provided hereinbelow with reference to the sequence listing part. Nucleotide sequence SEQ ID:284 is located at position 173577 relative to the genome of Vaccinia Virus.

VGAM298 precursor RNA folds onto itself, forming VGAM298 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM298 folded precursor RNA into VGAM298 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM298 RNA is designated SEQ ID:633, and is provided hereinbelow with reference to the sequence listing part.

VGAM298 host target gene, herein designated VGAM HOST TARGET GENE, encodes a corresponding messenger RNA, VGAM298 host target RNA, herein designated VGAM HOST TARGET RNA. VGAM298 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM298 RNA, herein designated VGAM RNA, binds complementarily to one or more host target binding sites located in untranslated regions of VGAM298 host target RNA, herein designated VGAM HOST TARGET RNA. This complementary binding is due to the fact that the nucleotide sequence of VGAM298 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM298 RNA, herein designated VGAM RNA, may have a different number of host target binding sites in untranslated regions of a VGAM298 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM298 RNA, herein designated VGAM RNA, to host target binding sites on VGAM298 host target RNA, herein designated VGAM HOST TARGET RNA, such as BINDING SITE I, BINDING SITE II and BINDING SITE III, inhibits translation of VGAM298 host target RNA into VGAM298 host target protein, herein designated VGAM HOST TARGET PROTEIN. VGAM host target protein is therefore outlined by a broken line.

It is appreciated that VGAM298 host target gene, herein designated VGAM HOST TARGET GENE, in fact represents a plurality of VGAM298 host target genes. The mRNA of each one of this plurality of VGAM298 host target genes comprises one or more host target binding sites, each having a nucleotide sequence which is at least partly complementary to VGAM298 RNA, herein designated VGAM RNA, and which when bound by VGAM298 RNA causes inhibition of translation of respective one or more VGAM298 host target proteins.

It is further appreciated by one skilled in the art that the mode of translational inhibition illustrated by FIG. 1 with specific reference to translational inhibition exerted by VGAM298 gene, herein designated VGAM GENE, on one or more VGAM298 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM298 is inhibition of expression of host target genes, as part of a novel viral mechanism of attacking a host. Accordingly, utilities of VGAM298 include diagnosis, prevention and treatment of viral infection by Vaccinia Virus. Specific functions, and accordingly utilities, of VGAM298 correlate with, and may be deduced from, the identity of the host target genes which VGAM298 binds and inhibits, and the function of these host target genes, as elaborated hereinbelow.

Nucleotide sequences of the VGAM298 precursor RNA, herein designated VGAM PRECURSOR RNA, and of the ‘diced’ VGAM298 RNA, herein designated VGAM RNA, and a schematic representation of the secondary folding of VGAM298 folded precursor RNA, herein designated VGAM FOLDED PRECURSOR RNA, of VGAM298 are further described hereinbelow with reference to Table 1.

Nucleotide sequences of host target binding sites, such as BINDING SITE-I, BINDING SITE-II and BINDING SITE-III of FIG. 1, found on VGAM298 host target RNA, and schematic representation of the complementarity of each of these host target binding sites to VGAM298 RNA, herein designated VGAM RNA, are described hereinbelow with reference to Table 2.

As mentioned hereinabove with reference to FIG. 1, a function of VGAM298 gene, herein designated VGAM is inhibition of expression of VGAM298 target genes. It is appreciated that specific functions, and accordingly utilities, of VGAM298 correlate with, and may be deduced from, the identity of the target genes which VGAM298 binds and inhibits, and the function of these target genes, as elaborated hereinbelow.

G2 (Accession XM_{—}039515) is a VGAM298 host target gene. G2 BINDING SITE is HOST TARGET binding site found in the 5′ untranslated region of mRNA encoded by G2, corresponding to a HOST TARGET binding site such as BINDING SITE I, BINDING SITE II or BINDING SITE III. Table 2 illustrates the complementarity of the nucleotide sequences of G2 BINDING SITE, designated SEQ ID:2756, to the nucleotide sequence of VGAM298 RNA, herein designated VGAM RNA, also designated SEQ ID:633.

A function of VGAM298 is therefore inhibition of G2 (Accession XM_{—}039515). Accordingly, utilities of VGAM298 include diagnosis, prevention and treatment of diseases and clinical conditions associated with G2. RAS (RAD and GEM)-like GTP-binding (REM, Accession NM_{—}014012) is another VGAM298 host target gene. REM BINDING SITE is HOST TARGET binding site found in the 5′ untranslated region of mRNA encoded by REM, corresponding to a HOST TARGET binding site such as BINDING SITE I, BINDING SITE II or BINDING SITE III. Table 2 illustrates the complementarity of the nucleotide sequences of REM BINDING SITE, designated SEQ ID: 1462, to the nucleotide sequence of VGAM298 RNA, herein designated VGAM RNA, also designated SEQ ID:633.

Another function of VGAM298 is therefore inhibition of RAS (RAD and GEM)-like GTP-binding (REM, Accession NM_{—}014012). Accordingly, utilities of VGAM298 include diagnosis, prevention and treatment of diseases and clinical conditions associated with REM. LOC90321 (Accession XM_{—}030896) is another VGAM298 host target gene. LOC90321 BINDING SITE is HOST TARGET binding site found in the 3′ untranslated region of mRNA encoded by LOC90321, corresponding to a HOST TARGET binding site such as BINDING SITE I, BINDING SITE II or BINDING SITE III. Table 2 illustrates the complementarity of the nucleotide sequences of LOC90321 BINDING SITE, designated SEQ ID:2631, to the nucleotide sequence of VGAM298 RNA, herein designated VGAM RNA, also designated SEQ ID:633.

Another function of VGAM298 is therefore inhibition of LOC90321 (Accession XM_{—}030896). Accordingly, utilities of VGAM298 include diagnosis, prevention and treatment of diseases and clinical conditions associated with LOC90321. LOC91650 (Accession XM_{—}039853) is another VGAM298 host target gene. LOC91650 BINDING SITE is HOST TARGET binding site found in the 5′ untranslated region of mRNA encoded by LOC91650, corresponding to a HOST TARGET binding site such as BINDING SITE I, BINDING SITE II or BINDING SITE III. Table 2 illustrates the complementarity of the nucleotide sequences of LOC91650 BINDING SITE, designated SEQ ID:2766, to the nucleotide sequence of VGAM298 RNA, herein designated VGAM RNA, also designated SEQ ID:633.

Another function of VGAM298 is therefore inhibition of LOC91650 (Accession XM_{—}039853). Accordingly, utilities of VGAM298 include diagnosis, prevention and treatment of diseases and clinical conditions associated with LOC91650. FIG. 1 further provides a conceptual description of a novel bioinformatically detected viral gene of the present invention, referred to here as Viral Genomic Address Messenger 299 (VGAM299) viral gene, which modulates expression of respective host target genes thereof, the function and utility of which host target genes is known in the art.

VGAM299 is a novel bioinformatically detected regulatory, non protein coding, viral micro RNA (miRNA) gene. The method by which VGAM299 was detected is described hereinabove with reference to FIGS. 1-8.

VGAM299 gene, herein designated VGAM GENE, is a viral gene contained in the genome of Vaccinia Virus. VGAM299 host target gene, herein designated VGAM HOST TARGET GENE, is a human gene contained in the human genome.

VGAM299 gene encodes a VGAM299 precursor RNA, herein designated VGAM PRECURSOR RNA. Similar to other miRNA genes, and unlike most ordinary genes, VGAM299 precursor RNA does not encode a protein. A nucleotide sequence identical or highly similar to the nucleotide sequence of VGAM299 precursor RNA is designated SEQ ID:285, and is provided hereinbelow with reference to the sequence listing part. Nucleotide sequence SEQ ID:285 is located at position 168765 relative to the genome of Vaccinia Virus.

VGAM299 precursor RNA folds onto itself, forming VGAM299 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM299 folded precursor RNA into VGAM299 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 25%) nucleotide sequence of VGAM299 RNA is designated SEQ ID:634, and is provided hereinbelow with reference to the sequence listing part.

VGAM299 host target gene, herein designated VGAM HOST TARGET GENE, encodes a corresponding messenger RNA, VGAM299 host target RNA, herein designated VGAM HOST TARGET RNA. VGAM299 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM299 RNA, herein designated VGAM RNA, binds complementarily to one or more host target binding sites located in untranslated regions of VGAM299 host target RNA, herein designated VGAM HOST TARGET RNA. This complementary binding is due to the fact that the nucleotide sequence of VGAM299 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM299 RNA, herein designated VGAM RNA, may have a different number of host target binding sites in untranslated regions of a VGAM299 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM299 RNA, herein designated VGAM RNA, to host target binding sites on VGAM299 host target RNA, herein designated VGAM HOST TARGET RNA, such as BINDING SITE I, BINDING SITE II and BINDING SITE III, inhibits translation of VGAM299 host target RNA into VGAM299 host target protein, herein designated VGAM HOST TARGET PROTEIN. VGAM host target protein is therefore outlined by a broken line.

It is appreciated that VGAM299 host target gene, herein designated VGAM HOST TARGET GENE, in fact represents a plurality of VGAM299 host target genes. The mRNA of each one of this plurality of VGAM299 host target genes comprises one or more host target binding sites, each having a nucleotide sequence which is at least partly complementary to VGAM299 RNA, herein designated VGAM RNA, and which when bound by VGAM299 RNA causes inhibition of translation of respective one or more VGAM299 host target proteins.

It is further appreciated by one skilled in the art that the mode of translational inhibition illustrated by FIG. 1 with specific reference to translational inhibition exerted by VGAM299 gene, herein designated VGAM GENE, on one or more VGAM299 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM299 is inhibition of expression of host target genes, as part of a novel viral mechanism of attacking a host. Accordingly, utilities of VGAM299 include diagnosis, prevention and treatment of viral infection by Vaccinia Virus. Specific functions, and accordingly utilities, of VGAM299 correlate with, and may be deduced from, the identity of the host target genes which VGAM299 binds and inhibits, and the function of these host target genes, as elaborated hereinbelow.

Nucleotide sequences of the VGAM299 precursor RNA, herein designated VGAM PRECURSOR RNA, and of the ‘diced’ VGAM299 RNA, herein designated VGAM RNA, and a schematic representation of the secondary folding of VGAM299 folded precursor RNA, herein designated VGAM FOLDED PRECURSOR RNA, of VGAM299 are further described hereinbelow with reference to Table 1.

Nucleotide sequences of host target binding sites, such as BINDING SITE-I, BINDING SITE-II and BINDING SITE-III of FIG. 1, found on VGAM299 host target RNA, and schematic representation of the complementarity of each of these host target binding sites to VGAM299 RNA, herein designated VGAM RNA, are described hereinbelow with reference to Table 2.

As mentioned hereinabove with reference to FIG. 1, a function of VGAM299 gene, herein designated VGAM is inhibition of expression of VGAM299 target genes. It is appreciated that specific functions, and accordingly utilities, of VGAM299 correlate with, and may be deduced from, the identity of the target genes which VGAM299 binds and inhibits, and the function of these target genes, as elaborated hereinbelow.

CD28 Antigen (Tp44) (CD28, Accession NM_{—}006139) is a VGAM299 host target gene. CD28 BINDING SITE is HOST TARGET binding site found in the 3′ untranslated region of mRNA encoded by CD28, corresponding to a HOST TARGET binding site such as BINDING SITE I, BINDING SITE II or BINDING SITE III. Table 2 illustrates the complementarity of the nucleotide sequences of CD28 BINDING SITE, designated SEQ ID:1274, to the nucleotide sequence of VGAM299 RNA, herein designated VGAM RNA, also designated SEQ ID:634.

A function of VGAM299 is therefore inhibition of CD28 Antigen (Tp44) (CD28, Accession NM_{—}006139), a gene which possibly involved in t-cell activation. Accordingly, utilities of VGAM299 include diagnosis, prevention and treatment of diseases and clinical conditions associated with CD28. The function of CD28 has been established by previous studies. Monoclonal antibodies recognize 3 antigens, CD3 (OMIM Ref. No. 186790), CD2 (OMIM Ref. No. 186990), and CD28 (Tp44), that cause human T cells to proliferate in the presence of phorbol esters. Whereas CD3 appeared to be involved in transduction of the signal generated by antigen binding to the T-cell receptor, the role of the CD2 and CD28 antigens in physiologic proliferation was not understood. Aruffo and Seed (1987) isolated a cDNA clone encoding CD28 by a simple and highly efficient cloning strategy based on transient expression. In COS cells the CD28 encodes a highly glycosylated membrane protein with homology to the immunoglobulin super family. Animal model experiments lend further support to the function of CD28. CD28 undergoes tyrosine phosphorylation after interacting with its ligand, B7 (CD80; 112203). Phosphorylation of tyr173 (tyr170 in mouse) in the cytoplasmic domain of CD28 allows the recruitment of signaling proteins such as phosphatidylinositol 3-kinase (see OMIM Ref. No. PIK3R1; 171833), GRB2 (OMIM Ref. No. 108355), and GADS (GRAP2; 604518) via their SH2 domains. Okkenhaug et al. (2001) reconstituted CD28 knockout mice with transgenes encoding wildtype Cd28 or Cd28 carrying a tyr170-to-phe mutation. Mutant Cd28 did not bind to the SH2 domain of PIK3R1, resulting in diminished protein kinase B (OMIM Ref. No. 164730) activation. Mutant Cd28 was able to prevent the induction of anergy, to promote T-cell proliferation and interleukin-2 (IL2; 147680) secretion, and to provide B-cell help, but was unable to up-regulate expression of the prosurvival protein BCLXL (OMIM Ref. No. 600039). The defect in BCLXL up-regulation was correlated with increased susceptibility of the T cells to gamma radiation. Okkenhaug et al. (2001) suggested that other tyrosine residues or asn172 may be critical to functions not affected by the tyr170-to-phe mutation.

It is appreciated that the abovementioned animal model for CD28 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Aruffo, A.; Seed, B.: Molecular cloning of a CD28 cDNA by a high-efficiency COS cell expression system. Proc. Nat. Acad. Sci. 84: 8573-8577, 1987.; and
Okkenhaug, K.; Wu, L.; Garza, K. M.; La Rose, J.; Khoo, W.; Odermatt, B.; Mak, T. W.; Ohashi, P. S.; Rottapel, R.: A point mutation in CD28 distinguishes proliferative signals from sur.

Further studies establishing the function and utilities of CD28 are found in John Hopkins OMIM database record ID 186760, and in sited publications numbered 1323-95 and 1324-1327 listed in the bibliography section hereinbelow, which are also hereby incorporated by reference. ATP-binding Cassette, Sub-family A (ABC1), Member 6 (ABCA6, Accession NM_{—}080284) is another VGAM299 host target gene. ABCA6 BINDING SITE is HOST TARGET binding site found in the 3′ untranslated region of mRNA encoded by ABCA6, corresponding to a HOST TARGET binding site such as BINDING SITE I, BINDING SITE II or BINDING SITE III. Table 2 illustrates the complementarity of the nucleotide sequences of ABCA6 BINDING SITE, designated SEQ ID:2374, to the nucleotide sequence of VGAM299 RNA, herein designated VGAM RNA, also designated SEQ ID:634.

Another function of VGAM299 is therefore inhibition of ATP-binding Cassette, Sub-family A (ABC1), Member 6 (ABCA6, Accession NM_{—}080284). Accordingly, utilities of VGAM299 include diagnosis, prevention and treatment of diseases and clinical conditions associated with ABCA6. FLJ21302 (Accession NM_{—}022901) is another VGAM299 host target gene. FLJ21302 BINDING SITE is HOST TARGET binding site found in the 3′ untranslated region of mRNA encoded by FLJ21302, corresponding to a HOST TARGET binding site such as BINDING SITE I, BINDING SITE II or BINDING SITE III. Table 2 illustrates the complementarity of the nucleotide sequences of FLJ21302 BINDING SITE, designated SEQ ID:2021, to the nucleotide sequence of VGAM299 RNA, herein designated VGAM RNA, also designated SEQ ID:634.

Another function of VGAM299 is therefore inhibition of FLJ21302 (Accession NM_{—}022901). Accordingly, utilities of VGAM299 include diagnosis, prevention and treatment of diseases and clinical conditions associated with FLJ21302. Solute Carrier Family 7, (cationic amino acid transporter, y+ system) Member 11 (SLC7A11, Accession NM_{—}014331) is another VGAM299 host target gene. SLC7A11 BINDING SITE is HOST TARGET binding site found in the 3′ untranslated region of mRNA encoded by SLC7A11, corresponding to a HOST TARGET binding site such as BINDING SITE I, BINDING SITE II or BINDING SITE III. Table 2 illustrates the complementarity of the nucleotide sequences of SLC7A11 BINDING SITE, designated SEQ ID:1492, to the nucleotide sequence of VGAM299 RNA, herein designated VGAM RNA, also designated SEQ ID:634.

Another function of VGAM299 is therefore inhibition of Solute Carrier Family 7, (cationic amino acid transporter, y+ system) Member 11 (SLC7A11, Accession NM_{—}014331). Accordingly, utilities of VGAM299 include diagnosis, prevention and treatment of diseases and clinical conditions associated with SLC7A11. FIG. 1 further provides a conceptual description of a novel bioinformatically detected viral gene of the present invention, referred to here as Viral Genomic Address Messenger 300 (VGAM300) viral gene, which modulates expression of respective host target genes thereof, the function and utility of which host target genes is known in the art.

VGAM300 is a novel bioinformatically detected regulatory, non protein coding, viral micro RNA (miRNA) gene. The method by which VGAM300 was detected is described hereinabove with reference to FIGS. 1-8.

VGAM300 gene, herein designated VGAM GENE, is a viral gene contained in the genome of Vaccinia Virus. VGAM300 host target gene, herein designated VGAM HOST TARGET GENE, is a human gene contained in the human genome.

VGAM300 gene encodes a VGAM300 precursor RNA, herein designated VGAM PRECURSOR RNA. Similar to other miRNA genes, and unlike most ordinary genes, VGAM300 precursor RNA does not encode a protein. A nucleotide sequence identical or highly similar to the nucleotide sequence of VGAM300 precursor RNA is designated SEQ ID:286, and is provided hereinbelow with reference to the sequence listing part. Nucleotide sequence SEQ ID:286 is located at position 172477 relative to the genome of Vaccinia Virus.

VGAM300 precursor RNA folds onto itself, forming VGAM300 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM300 folded precursor RNA into VGAM300 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM300 RNA is designated SEQ ID:635, and is provided hereinbelow with reference to the sequence listing part.

VGAM300 host target gene, herein designated VGAM HOST TARGET GENE, encodes a corresponding messenger RNA, VGAM300 host target RNA, herein designated VGAM HOST TARGET RNA. VGAM300 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM300 RNA, herein designated VGAM RNA, binds complementarily to one or more host target binding sites located in untranslated regions of VGAM300 host target RNA, herein designated VGAM HOST TARGET RNA. This complementary binding is due to the fact that the nucleotide sequence of VGAM300 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM300 RNA, herein designated VGAM RNA, may have a different number of host target binding sites in untranslated regions of a VGAM300 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM300 RNA, herein designated VGAM RNA, to host target binding sites on VGAM300 host target RNA, herein designated VGAM HOST TARGET RNA, such as BINDING SITE I, BINDING SITE II and BINDING SITE III, inhibits translation of VGAM300 host target RNA into VGAM300 host target protein, herein designated VGAM HOST TARGET PROTEIN. VGAM host target protein is therefore outlined by a broken line.

It is appreciated that VGAM300 host target gene, herein designated VGAM HOST TARGET GENE, in fact represents a plurality of VGAM300 host target genes. The mRNA of each one of this plurality of VGAM300 host target genes comprises one or more host target binding sites, each having a nucleotide sequence which is at least partly complementary to VGAM300 RNA, herein designated VGAM RNA, and which when bound by VGAM300 RNA causes inhibition of translation of respective one or more VGAM300 host target proteins.

It is further appreciated by one skilled in the art that the mode of translational inhibition illustrated by FIG. 1 with specific reference to translational inhibition exerted by VGAM300 gene, herein designated VGAM GENE, on one or more VGAM300 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM300 is inhibition of expression of host target genes, as part of a novel viral mechanism of attacking a host. Accordingly, utilities of VGAM300 include diagnosis, prevention and treatment of viral infection by Vaccinia Virus. Specific functions, and accordingly utilities, of VGAM300 correlate with, and may be deduced from, the identity of the host target genes which VGAM300 binds and inhibits, and the function of these host target genes, as elaborated hereinbelow.

Nucleotide sequences of the VGAM300 precursor RNA, herein designated VGAM PRECURSOR RNA, and of the ‘diced’ VGAM300 RNA, herein designated VGAM RNA, and a schematic representation of the secondary folding of VGAM300 folded precursor RNA, herein designated VGAM FOLDED PRECURSOR RNA, of VGAM300 are further described hereinbelow with reference to Table 1.

Nucleotide sequences of host target binding sites, such as BINDING SITE-I, BINDING SITE-II and BINDING SITE-III of FIG. 1, found on VGAM300 host target RNA, and schematic representation of the complementarity of each of these host target binding sites to VGAM300 RNA, herein designated VGAM RNA, are described hereinbelow with reference to Table 2.

As mentioned hereinabove with reference to FIG. 1, a function of VGAM300 gene, herein designated VGAM is inhibition of expression of VGAM300 target genes. It is appreciated that specific functions, and accordingly utilities, of VGAM300 correlate with, and may be deduced from, the identity of the target genes which VGAM300 binds and inhibits, and the function of these target genes, as elaborated hereinbelow.

Adaptor-related Protein Complex 2, Beta 1 Subunit (AP2B1, Accession NM_{—}001282) is a VGAM300 host target gene. AP2B1 BINDING SITE is HOST TARGET binding site found in the 3′ untranslated region of mRNA encoded by AP2B1, corresponding to a HOST TARGET binding site such as BINDING SITE I, BINDING SITE II or BINDING SITE III. Table 2 illustrates the complementarity of the nucleotide sequences of AP2B1 BINDING SITE, designated SEQ ID:814, to the nucleotide sequence of VGAM300 RNA, herein designated VGAM RNA, also designated SEQ ID:635.

A function of VGAM300 is therefore inhibition of Adaptor-related Protein Complex 2, Beta 1 Subunit (AP2B1, Accession NM_{—}001282), a gene which links clathrin to receptors in coated vesicles. Accordingly, utilities of VGAM300 include diagnosis, prevention and treatment of diseases and clinical conditions associated with AP2B1. The function of AP2B1 has been established by previous studies. The beta adaptin subunit of the clathrin coat assembly complex, also referred to as AP2-beta, was cloned from human, rat and bovine cDNA libraries by Ponnambalam et al. (1990) who found that the predicted 937-amino acid proteins are totally conserved between species. The protein is part of the AP2 coat assembly protein complex (see OMIM Ref. No. 601024) and links clathrin (OMIM Ref. No. 118960) to receptors in the coated vesicles. Druck et al. (1995) used a probe from the 3-prime UTR of the human cDNA to map the gene to chromosome 17. Hybrids with portions of chromosome 17 were then used to localize CLAPB1 to 17q11.2-q12.

Full details of the abovementioned studies are described in the following publications, the disclosure of which are hereby incorporated by reference:
Druck, T.; Gu, Y.; Prabhala. G.; Cannizzaro, L. A.; Park, S.-H.; Huebner, K.; Keen, J. H.: Chromosome localization of human genes for clathrin adaptor polypeptides AP2-beta and AP50 and the clathrin-binding protein, VCP. Genomics 30: 94-97, 1995.; and
Ponnambalam, S.; Robinson, M. S.; Jackson, A. P.; Peiperl, L.; Parham, P.: Conservation and diversity in families of coated vesicle adaptins. J. Biol. Chem. 265: 4814-4820, 1990.

Further studies establishing the function and utilities of AP2B1 are found in John Hopkins OMIM database record ID 601025, and in sited publications numbered 2239-2240 listed in the bibliography section hereinbelow, which are also hereby incorporated by reference. Glutamic-oxaloacetic Transaminase 1, Soluble (aspartate aminotransferase 1) (GOT1, Accession NM_{—}002079) is another VGAM300 host target gene. GOT1 BINDING SITE is HOST TARGET binding site found in the 3′ untranslated region of mRNA encoded by GOT1, corresponding to a HOST TARGET binding site such as BINDING SITE I, BINDING SITE II or BINDING SITE III. Table 2 illustrates the complementarity of the nucleotide sequences of GOT1 BINDING SITE, designated SEQ ID:897, to the nucleotide sequence of VGAM300 RNA, herein designated VGAM RNA, also designated SEQ ID:635.

Another function of VGAM300 is therefore inhibition of Glutamic-oxaloacetic Transaminase 1, Soluble (aspartate aminotransferase 1) (GOT1, Accession NM_{—}002079), a gene which reversibly transfers amino group from aspartate to 2-oxoglutarate to form oxaloacetate and glutamate. Accordingly, utilities of VGAM300 include diagnosis, prevention and treatment of diseases and clinical conditions associated with GOT1. The function of GOT1 has been established by previous studies. Glutamate oxaloacetate transaminase (EC 2.6.1.1) is a ubiquitous pyridoxal phosphate-dependent enzyme which exists in both mitochondrial (OMIM Ref. No. 138150) and cytosolic forms. The enzyme plays an important role in amino acid metabolism and in the urea and tricarboxylic acid cycles. The 2 isoenzymes are homodimeric. In liver about 80% of the enzyme activity is mitochondrial in origin, whereas in serum the enzyme activity is largely cytosolic. Although the mitochondrial and soluble forms of GOT are coded by different chromosomes (according to a rule that has few exceptions; McKusick, 1986), the 2 show close homology in amino acid sequence and were presumably derived from a common ancestral gene (Ford et al., 1980; Doonan et al., 1984). Panteghini (1990) reviewed the clinical usefulness of assays for aspartate aminotransferase (AST) isoenzymes in serum. By analysis of mouse-human somatic cell hybrids, Creagan et al. (1973) concluded that the structural locus for cytoplasmic glutamate oxaloacetate transaminase is on chromosome 10. Spritz et al. (1979) studied soluble GOT activity in fibroblasts of 2 persons with duplications of the long arm of chromosome 10. Since the 2 differed by only half a band, the authors concluded that the structural locus is on band 10q24. Koch et al. (1981) pointed out that GOT1 and LIPA (OMIM Ref. No. 278000) are also syntenic on chromosome 19 of the mouse. Junien et al. (1982) assigned GOT1 and PGAMA (OMIM Ref. No. 172250) to 10q26.1 (or 10q25.3) by dosage studies. Pol et al. (1988) cloned cDNAs corresponding to human liver cytosolic and mitochondrial aspartate aminotransferase mRNAs. Pol et al. (1989) used these cDNA probes to locate the GOT1 gene in the region 10q24.1-q25.1 by in situ hybridization. Wang et al. (1999) located the GOT1 gene within the critical region for the urofacial syndrome (OMIM Ref. No. 236730), between markers D10S198 and D10S2494, but excluded it as a candidate for that disorder by mutation analysis.

Full details of the abovementioned studies are described in the following publications, the disclosure of which are hereby incorporated by reference:
McKusick, V. A.: The morbid anatomy of the human genome: a review of gene mapping in clinical medicine (part 1). Medicine 65: 1-33, 1986.; and
Wang, C.-Y.; Huang, Y.-Q.; Shi, J.-O.; Marron, M. P.; Ruan, Q.-G.; Hawkins-Lee, B.; Ochoa, B.; She, J.-X.: Genetic homogeneity, high-resolution mapping, and mutation analysis of the u.

Further studies establishing the function and utilities of GOT1 are found in John Hopkins OMIM database record ID 138180, and in sited publications numbered 2757-2758, 690, 2759-2761, 2763, 2767-2769, 402, 88 and 2770-2774 listed in the bibliography section hereinbelow, which are also hereby incorporated by reference. Zinc Finger Protein 14 (KOX 6) (ZNF14, Accession NM_{—}021030) is another VGAM300 host target gene. ZNF14 BINDING SITE is HOST TARGET binding site found in the 3′ untranslated region of mRNA encoded by ZNF14, corresponding to a HOST TARGET binding site such as BINDING SITE I, BINDING SITE II or BINDING SITE III. Table 2 illustrates the complementarity of the nucleotide sequences of ZNF14 BINDING SITE, designated SEQ ID:1934, to the nucleotide sequence of VGAM300 RNA, herein designated VGAM RNA, also designated SEQ ID:635.

Another function of VGAM300 is therefore inhibition of Zinc Finger Protein 14 (KOX 6) (ZNF14, Accession NM_{—}021030). Accordingly, utilities of VGAM300 include diagnosis, prevention and treatment of diseases and clinical conditions associated with ZNF14. FLJ11827 (Accession NM_{—}025093) is another VGAM300 host target gene. FLJ11827 BINDING SITE is HOST TARGET binding site found in the 3′ untranslated region of mRNA encoded by FLJ11827, corresponding to a HOST TARGET binding site such as BINDING SITE I, BINDING SITE II or BINDING SITE III. Table 2 illustrates the complementarity of the nucleotide sequences of FLJ11827 BINDING SITE, designated SEQ ID:2136, to the nucleotide sequence of VGAM300 RNA, herein designated VGAM RNA, also designated SEQ ID:635.

Another function of VGAM300 is therefore inhibition of FLJ11827 (Accession NM_{—}025093). Accordingly, utilities of VGAM300 include diagnosis, prevention and treatment of diseases and clinical conditions associated with FLJ11827. Hairy/enhancer-of-split Related with YRPW Motif-like (HEYL, Accession NM_{—}014571) is another VGAM300 host target gene. HEYL BINDING SITE is HOST TARGET binding site found in the 3′ untranslated region of mRNA encoded by HEYL, corresponding to a HOST TARGET binding site such as BINDING SITE I, BINDING SITE II or BINDING SITE III. Table 2 illustrates the complementarity of the nucleotide sequences of HEYL BINDING SITE, designated SEQ ID:1507, to the nucleotide sequence of VGAM300 RNA, herein designated VGAM RNA, also designated SEQ ID:635.

Another function of VGAM300 is therefore inhibition of Hairy/enhancer-of-split Related with YRPW Motif-like (HEYL, Accession NM_{—}014571). Accordingly, utilities of VGAM300 include diagnosis, prevention and treatment of diseases and clinical conditions associated with HEYL. LOC158722 (Accession XM_{—}088653) is another VGAM300 host target gene. LOC158722 BINDING SITE is HOST TARGET binding site found in the 5′ untranslated region of mRNA encoded by LOC158722, corresponding to a HOST TARGET binding site such as BINDING SITE I, BINDING SITE II or BINDING SITE III. Table 2 illustrates the complementarity of the nucleotide sequences of LOC158722 BINDING SITE, designated SEQ ID:3226, to the nucleotide sequence of VGAM300 RNA, herein designated VGAM RNA, also designated SEQ ID:635.

Another function of VGAM300 is therefore inhibition of LOC158722 (Accession XM_{—}088653). Accordingly, utilities of VGAM300 include diagnosis, prevention and treatment of diseases and clinical conditions associated with LOC158722. FIG. 1 further provides a conceptual description of a novel bioinformatically detected viral gene of the present invention, referred to here as Viral Genomic Address Messenger 301 (VGAM301) viral gene, which modulates expression of respective host target genes thereof, the function and utility of which host target genes is known in the art.

VGAM301 is a novel bioinformatically detected regulatory, non protein coding, viral micro RNA (miRNA) gene. The method by which VGAM301 was detected is described hereinabove with reference to FIGS. 1-8.

VGAM301 gene, herein designated VGAM GENE, is a viral gene contained in the genome of Vaccinia Virus. VGAM301 host target gene, herein designated VGAM HOST TARGET GENE, is a human gene contained in the human genome.

VGAM301 gene encodes a VGAM301 precursor RNA, herein designated VGAM PRECURSOR RNA. Similar to other miRNA genes, and unlike most ordinary genes, VGAM301 precursor RNA does not encode a protein. A nucleotide sequence identical or highly similar to the nucleotide sequence of VGAM301 precursor RNA is designated SEQ ID:287, and is provided hereinbelow with reference to the sequence listing part. Nucleotide sequence SEQ ID:287 is located at position 174245 relative to the genome of Vaccinia Virus.

VGAM301 precursor RNA folds onto itself, forming VGAM301 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM301 folded precursor RNA into VGAM301 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM301 RNA is designated SEQ ID:636, and is provided hereinbelow with reference to the sequence listing part.

VGAM301 host target gene, herein designated VGAM HOST TARGET GENE, encodes a corresponding messenger RNA, VGAM301 host target RNA, herein designated VGAM HOST TARGET RNA. VGAM301 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM301 RNA, herein designated VGAM RNA, binds complementarily to one or more host target binding sites located in untranslated regions of VGAM301 host target RNA, herein designated VGAM HOST TARGET RNA. This complementary binding is due to the fact that the nucleotide sequence of VGAM301 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM301 RNA, herein designated VGAM RNA, may have a different number of host target binding sites in untranslated regions of a VGAM301 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM301 RNA, herein designated VGAM RNA, to host target binding sites on VGAM301 host target RNA, herein designated VGAM HOST TARGET RNA, such as BINDING SITE I, BINDING SITE II and BINDING SITE III, inhibits translation of VGAM301 host target RNA into VGAM301 host target protein, herein designated VGAM HOST TARGET PROTEIN. VGAM host target protein is therefore outlined by a broken line.

It is appreciated that VGAM301 host target gene, herein designated VGAM HOST TARGET GENE, in fact represents a plurality of VGAM301 host target genes. The mRNA of each one of this plurality of VGAM301 host target genes comprises one or more host target binding sites, each having a nucleotide sequence which is at least partly complementary to VGAM301 RNA, herein designated VGAM RNA, and which when bound by VGAM301 RNA causes inhibition of translation of respective one or more VGAM301 host target proteins.

It is further appreciated by one skilled in the art that the mode of translational inhibition illustrated by FIG. 1 with specific reference to translational inhibition exerted by VGAM301 gene, herein designated VGAM GENE, on one or more VGAM301 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM301 is inhibition of expression of host target genes, as part of a novel viral mechanism of attacking a host. Accordingly, utilities of VGAM301 include diagnosis, prevention and treatment of viral infection by Vaccinia Virus. Specific functions, and accordingly utilities, of VGAM301 correlate with, and may be deduced from, the identity of the host target genes which VGAM301 binds and inhibits, and the function of these host target genes, as elaborated hereinbelow.

Nucleotide sequences of the VGAM301 precursor RNA, herein designated VGAM PRECURSOR RNA, and of the ‘diced’ VGAM301 RNA, herein designated VGAM RNA, and a schematic representation of the secondary folding of VGAM301 folded precursor RNA, herein designated VGAM FOLDED PRECURSOR RNA, of VGAM301 are further described hereinbelow with reference to Table 1.

Nucleotide sequences of host target binding sites, such as BINDING SITE-I, BINDING SITE-II and BINDING SITE-III of FIG. 1, found on VGAM301 host target RNA, and schematic representation of the complementarity of each of these host target binding sites to VGAM301 RNA, herein designated VGAM RNA, are described hereinbelow with reference to Table 2.

As mentioned hereinabove with reference to FIG. 1, a function of VGAM301 gene, herein designated VGAM is inhibition of expression of VGAM301 target genes. It is appreciated that specific functions, and accordingly utilities, of VGAM301 correlate with, and may be deduced from, the identity of the target genes which VGAM301 binds and inhibits, and the function of these target genes, as elaborated hereinbelow.

A Disintegrin-like and Metalloprotease (reprolysin type) with Thrombospondin Type 1 Motif, 5 (aggrecanase-2) (ADAMTS5, Accession NM_{—}007038) is a VGAM301 host target gene. ADAMTS5 BINDING SITE is HOST TARGET binding site found in the 3′ untranslated region of mRNA encoded by ADAMTS5, corresponding to a HOST TARGET binding site such as BINDING SITE I, BINDING SITE II or BINDING SITE III. Table 2 illustrates the complementarity of the nucleotide sequences of ADAMTS5 BINDING SITE, designated SEQ ID:1350, to the nucleotide sequence of VGAM301 RNA, herein designated VGAM RNA, also designated SEQ ID:636.

A function of VGAM301 is therefore inhibition of A Disintegrin-like and Metalloprotease (reprolysin type) with Thrombospondin Type 1 Motif, 5 (aggrecanase-2) (ADAMTS5, Accession NM_{—}007038), a gene which cleaves aggrecan, a cartilage proteoglycan, and may be involved in its turnover. Accordingly, utilities of VGAM301 include diagnosis, prevention and treatment of diseases and clinical conditions associated with ADAMTS5. The function of ADAMTS5 and its association with various diseases and clinical conditions, has been established by previous studies, as described hereinabove with reference to VGAM49. Solute Carrier Family 38, Member 4 (SLC38A4, Accession NM_{—}018018) is another VGAM301 host target gene. SLC38A4 BINDING SITE is HOST TARGET binding site found in the 3′ untranslated region of mRNA encoded by SLC38A4, corresponding to a HOST TARGET binding site such as BINDING SITE I, BINDING SITE II or BINDING SITE III. Table 2 illustrates the complementarity of the nucleotide sequences of SLC38A4 BINDING SITE, designated SEQ ID:1774, to the nucleotide sequence of VGAM301 RNA, herein designated VGAM RNA, also designated SEQ ID:636.

Another function of VGAM301 is therefore inhibition of Solute Carrier Family 38, Member 4 (SLC38A4, Accession NM_{—}018018). Accordingly, utilities of VGAM301 include diagnosis, prevention and treatment of diseases and clinical conditions associated with SLC38A4. FIG. 1 further provides a conceptual description of a novel bioinformatically detected viral gene of the present invention, referred to here as Viral Genomic Address Messenger 302 (VGAM302) viral gene, which modulates expression of respective host target genes thereof, the function and utility of which host target genes is known in the art.

VGAM302 is a novel bioinformatically detected regulatory, non protein coding, viral micro RNA (miRNA) gene. The method by which VGAM302 was detected is described hereinabove with reference to FIGS. 1-8.

VGAM302 gene, herein designated VGAM GENE, is a viral gene contained in the genome of Vaccinia Virus. VGAM302 host target gene, herein designated VGAM HOST TARGET GENE, is a human gene contained in the human genome.

VGAM302 gene encodes a VGAM302 precursor RNA, herein designated VGAM PRECURSOR RNA. Similar to other miRNA genes, and unlike most ordinary genes, VGAM302 precursor RNA does not encode a protein. A nucleotide sequence identical or highly similar to the nucleotide sequence of VGAM302 precursor RNA is designated SEQ ID:288, and is provided hereinbelow with reference to the sequence listing part. Nucleotide sequence SEQ ID:288 is located at position 175500 relative to the genome of Vaccinia Virus.

VGAM302 precursor RNA folds onto itself, forming VGAM302 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM302 folded precursor RNA into VGAM302 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM302 RNA is designated SEQ ID:637, and is provided hereinbelow with reference to the sequence listing part.

VGAM302 host target gene, herein designated VGAM HOST TARGET GENE, encodes a corresponding messenger RNA, VGAM302 host target RNA, herein designated VGAM HOST TARGET RNA. VGAM302 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM302 RNA, herein designated VGAM RNA, binds complementarily to one or more host target binding sites located in untranslated regions of VGAM302 host target RNA, herein designated VGAM HOST TARGET RNA. This complementary binding is due to the fact that the nucleotide sequence of VGAM302 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM302 RNA, herein designated VGAM RNA, may have a different number of host target binding sites in untranslated regions of a VGAM302 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM302 RNA, herein designated VGAM RNA, to host target binding sites on VGAM302 host target RNA, herein designated VGAM HOST TARGET RNA, such as BINDING SITE I, BINDING SITE II and BINDING SITE III, inhibits translation of VGAM302 host target RNA into VGAM302 host target protein, herein designated VGAM HOST TARGET PROTEIN. VGAM host target protein is therefore outlined by a broken line.

It is appreciated that VGAM302 host target gene, herein designated VGAM HOST TARGET GENE, in fact represents a plurality of VGAM302 host target genes. The mRNA of each one of this plurality of VGAM302 host target genes comprises one or more host target binding sites, each having a nucleotide sequence which is at least partly complementary to VGAM302 RNA, herein designated VGAM RNA, and which when bound by VGAM302 RNA causes inhibition of translation of respective one or more VGAM302 host target proteins.

It is further appreciated by one skilled in the art that the mode of translational inhibition illustrated by FIG. 1 with specific reference to translational inhibition exerted by VGAM302 gene, herein designated VGAM GENE, on one or more VGAM302 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM302 is inhibition of expression of host target genes, as part of a novel viral mechanism of attacking a host. Accordingly, utilities of VGAM302 include diagnosis, prevention and treatment of viral infection by Vaccinia Virus. Specific functions, and accordingly utilities, of VGAM302 correlate with, and may be deduced from, the identity of the host target genes which VGAM302 binds and inhibits, and the function of these host target genes, as elaborated hereinbelow.

Nucleotide sequences of the VGAM302 precursor RNA, herein designated VGAM PRECURSOR RNA, and of the ‘diced’ VGAM302 RNA, herein designated VGAM RNA, and a schematic representation of the secondary folding of VGAM302 folded precursor RNA, herein designated VGAM FOLDED PRECURSOR RNA, of VGAM302 are further described hereinbelow with reference to Table 1.

Nucleotide sequences of host target binding sites, such as BINDING SITE-I, BINDING SITE-II and BINDING SITE-III of FIG. 1, found on VGAM302 host target RNA, and schematic representation of the complementarity of each of these host target binding sites to VGAM302 RNA, herein designated VGAM RNA, are described hereinbelow with reference to Table 2.

As mentioned hereinabove with reference to FIG. 1, a function of VGAM302 gene, herein designated VGAM is inhibition of expression of VGAM302 target genes. It is appreciated that specific functions, and accordingly utilities, of VGAM302 correlate with, and may be deduced from, the identity of the target genes which VGAM302 binds and inhibits, and the function of these target genes, as elaborated hereinbelow.

Solute Carrier Family 19 (thiamine transporter), Member 2 (SLC19A2, Accession XM_{—}044421) is a VGAM302 host target gene. SLC19A2 BINDING SITE is HOST TARGET binding site found in the 3′ untranslated region of mRNA encoded by SLC19A2, corresponding to a HOST TARGET binding site such as BINDING SITE I, BINDING SITE II or BINDING SITE III. Table 2 illustrates the complementarity of the nucleotide sequences of SLC19A2 BINDING SITE, designated SEQ ID:2835, to the nucleotide sequence of VGAM302 RNA, herein designated VGAM RNA, also designated SEQ ID:637.

A function of VGAM302 is therefore inhibition of Solute Carrier Family 19 (thiamine transporter), Member 2 (SLC19A2, Accession XM_{—}044421). Accordingly, utilities of VGAM302 include diagnosis, prevention and treatment of diseases and clinical conditions associated with SLC19A2. Collectin Sub-family Member 12 (COLEC12, Accession NM_{—}030781) is another VGAM302 host target gene. COLEC12 BINDING SITE1 and COLEC12 BINDING SITE2 are HOST TARGET binding sites found in untranslated regions of mRNA encoded by COLEC12, corresponding to HOST TARGET binding sites such as BINDING SITE I, BINDING SITE II or BINDING SITE III. Table 2 illustrates the complementarity of the nucleotide sequences of COLEC12 BINDING SITE1 and COLEC12 BINDING SITE2, designated SEQ ID:2165 and SEQ ID:2395 respectively, to the nucleotide sequence of VGAM302 RNA, herein designated VGAM RNA, also designated SEQ ID:637.

Another function of VGAM302 is therefore inhibition of Collectin Sub-family Member 12 (COLEC12, Accession NM_{—}030781). Accordingly, utilities of VGAM302 include diagnosis, prevention and treatment of diseases and clinical conditions associated with COLEC12. LOC150848 (Accession XM_{—}097959) is another VGAM302 host target gene. LOC150848 BINDING SITE is HOST TARGET binding site found in the 5′ untranslated region of mRNA encoded by LOC150848, corresponding to a HOST TARGET binding site such as BINDING SITE I, BINDING SITE II or BINDING SITE III. Table 2 illustrates the complementarity of the nucleotide sequences of LOC150848 BINDING SITE, designated SEQ ID:3334, to the nucleotide sequence of VGAM302 RNA, herein designated VGAM RNA, also designated SEQ ID:637.

Another function of VGAM302 is therefore inhibition of LOC150848 (Accession XM_{—}097959). Accordingly, utilities of VGAM302 include diagnosis, prevention and treatment of diseases and clinical conditions associated with LOC150848. LOC219846 (Accession XM_{—}165591) is another VGAM302 host target gene. LOC219846 BINDING SITE is HOST TARGET binding site found in the 3′ untranslated region of mRNA encoded by LOC219846, corresponding to a HOST TARGET binding site such as BINDING SITE I, BINDING SITE II or BINDING SITE III. Table 2 illustrates the complementarity of the nucleotide sequences of LOC219846 BINDING SITE, designated SEQ ID:3504, to the nucleotide sequence of VGAM302 RNA, herein designated VGAM RNA, also designated SEQ ID:637.

Another function of VGAM302 is therefore inhibition of LOC219846 (Accession XM_{—}165591). Accordingly, utilities of VGAM302 include diagnosis, prevention and treatment of diseases and clinical conditions associated with LOC219846. FIG. 1 further provides a conceptual description of a novel bioinformatically detected viral gene of the present invention, referred to here as Viral Genomic Address Messenger 303 (VGAM303) viral gene, which modulates expression of respective host target genes thereof, the function and utility of which host target genes is known in the art.

VGAM303 is a novel bioinformatically detected regulatory, non protein coding, viral micro RNA (miRNA) gene. The method by which VGAM303 was detected is described hereinabove with reference to FIGS. 1-8.

VGAM303 gene, herein designated VGAM GENE, is a viral gene contained in the genome of Vaccinia Virus. VGAM303 host target gene, herein designated VGAM HOST TARGET GENE, is a human gene contained in the human genome.

VGAM303 gene encodes a VGAM303 precursor RNA, herein designated VGAM PRECURSOR RNA. Similar to other miRNA genes, and unlike most ordinary genes, VGAM303 precursor RNA does not encode a protein. A nucleotide sequence identical or highly similar to the nucleotide sequence of VGAM303 precursor RNA is designated SEQ ID:289, and is provided hereinbelow with reference to the sequence listing part. Nucleotide sequence SEQ ID:289 is located at position 9636 relative to the genome of Vaccinia Virus.

VGAM303 precursor RNA folds onto itself, forming VGAM303 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM303 folded precursor RNA into VGAM303 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM303 RNA is designated SEQ ID:638, and is provided hereinbelow with reference to the sequence listing part.

VGAM303 host target gene, herein designated VGAM HOST TARGET GENE, encodes a corresponding messenger RNA, VGAM303 host target RNA, herein designated VGAM HOST TARGET RNA. VGAM303 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM303 RNA, herein designated VGAM RNA, binds complementarily to one or more host target binding sites located in untranslated regions of VGAM303 host target RNA, herein designated VGAM HOST TARGET RNA. This complementary binding is due to the fact that the nucleotide sequence of VGAM303 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM303 RNA, herein designated VGAM RNA, may have a different number of host target binding sites in untranslated regions of a VGAM303 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM303 RNA, herein designated VGAM RNA, to host target binding sites on VGAM303 host target RNA, herein designated VGAM HOST TARGET RNA, such as BINDING SITE I, BINDING SITE II and BINDING SITE III, inhibits translation of VGAM303 host target RNA into VGAM303 host target protein, herein designated VGAM HOST TARGET PROTEIN. VGAM host target protein is therefore outlined by a broken line.

It is appreciated that VGAM303 host target gene, herein designated VGAM HOST TARGET GENE, in fact represents a plurality of VGAM303 host target genes. The mRNA of each one of this plurality of VGAM303 host target genes comprises one or more host target binding sites, each having a nucleotide sequence which is at least partly complementary to VGAM303 RNA, herein designated VGAM RNA, and which when bound by VGAM303 RNA causes inhibition of translation of respective one or more VGAM303 host target proteins.

It is further appreciated by one skilled in the art that the mode of translational inhibition illustrated by FIG. 1 with specific reference to translational inhibition exerted by VGAM303 gene, herein designated VGAM GENE, on one or more VGAM303 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM303 is inhibition of expression of host target genes, as part of a novel viral mechanism of attacking a host. Accordingly, utilities of VGAM303 include diagnosis, prevention and treatment of viral infection by Vaccinia Virus. Specific functions, and accordingly utilities, of VGAM303 correlate with, and may be deduced from, the identity of the host target genes which VGAM303 binds and inhibits, and the function of these host target genes, as elaborated hereinbelow.

Nucleotide sequences of the VGAM303 precursor RNA, herein designated VGAM PRECURSOR RNA, and of the ‘diced’ VGAM303 RNA, herein designated VGAM RNA, and a schematic representation of the secondary folding of VGAM303 folded precursor RNA, herein designated VGAM FOLDED PRECURSOR RNA, of VGAM303 are further described hereinbelow with reference to Table 1.

Nucleotide sequences of host target binding sites, such as BINDING SITE-I, BINDING SITE-II and BINDING SITE-III of FIG. 1, found on VGAM303 host target RNA, and schematic representation of the complementarity of each of these host target binding sites to VGAM303 RNA, herein designated VGAM RNA, are described hereinbelow with reference to Table 2.

As mentioned hereinabove with reference to FIG. 1, a function of VGAM303 gene, herein designated VGAM is inhibition of expression of VGAM303 target genes. It is appreciated that specific functions, and accordingly utilities, of VGAM303 correlate with, and may be deduced from, the identity of the target genes which VGAM303 binds and inhibits, and the function of these target genes, as elaborated hereinbelow.

Dopamine Receptor D1 (DRD1, Accession NM_{—}000794) is a VGAM303 host target gene. DRD1 BINDING SITE is HOST TARGET binding site found in the 3′ untranslated region of mRNA encoded by DRD1, corresponding to a HOST TARGET binding site such as BINDING SITE I, BINDING SITE II or BINDING SITE III. Table 2 illustrates the complementarity of the nucleotide sequences of DRD1 BINDING SITE, designated SEQ ID:774, to the nucleotide sequence of VGAM303 RNA, herein designated VGAM RNA, also designated SEQ ID:638.

A function of VGAM303 is therefore inhibition of Dopamine Receptor D1 (DRD1, Accession NM_{—}000794), a gene which is mediated by g proteins which activate adenylyl cyclase. Accordingly, utilities of VGAM303 include diagnosis, prevention and treatment of diseases and clinical conditions associated with DRD1. The function of DRD1 and its association with various diseases and clinical conditions, has been established by previous studies, as described hereinabove with reference to VGAM58. KIAA0182 (Accession XM_{—}050495) is another VGAM303 host target gene. KIAA0182 BINDING SITE is HOST TARGET binding site found in the 3′ untranslated region of mRNA encoded by KIAA0182, corresponding to a HOST TARGET binding site such as BINDING SITE I, BINDING SITE II or BINDING SITE III. Table 2 illustrates the complementarity of the nucleotide sequences of KIAA0182 BINDING SITE, designated SEQ ID:2931, to the nucleotide sequence of VGAM303 RNA, herein designated VGAM RNA, also designated SEQ ID:638.

Another function of VGAM303 is therefore inhibition of KIAA0182 (Accession XM_{—}050495). Accordingly, utilities of VGAM303 include diagnosis, prevention and treatment of diseases and clinical conditions associated with KIAA0182. SSH2 (Accession XM_{—}030846) is another VGAM303 host target gene. SSH2 BINDING SITE is HOST TARGET binding site found in the 3′ untranslated region of mRNA encoded by SSH2, corresponding to a HOST TARGET binding site such as BINDING SITE I, BINDING SITE II or BINDING SITE III. Table 2 illustrates the complementarity of the nucleotide sequences of SSH2 BINDING SITE, designated SEQ ID:2626, to the nucleotide sequence of VGAM303 RNA, herein designated VGAM RNA, also designated SEQ ID:638.

Another function of VGAM303 is therefore inhibition of SSH2 (Accession XM_{—}030846). Accordingly, utilities of VGAM303 include diagnosis, prevention and treatment of diseases and clinical conditions associated with SSH2. LOC90918 (Accession XM_{—}034863) is another VGAM303 host target gene. LOC90918 BINDING SITE is HOST TARGET binding site found in the 5′ untranslated region of mRNA encoded by LOC90918, corresponding to a HOST TARGET binding site such as BINDING SITE I, BINDING SITE II or BINDING SITE III. Table 2 illustrates the complementarity of the nucleotide sequences of LOC90918 BINDING SITE, designated SEQ ID:2687, to the nucleotide sequence of VGAM303 RNA, herein designated VGAM RNA, also designated SEQ ID:638.

Another function of VGAM303 is therefore inhibition of LOC90918 (Accession XM_{—}034863). Accordingly, utilities of VGAM303 include diagnosis, prevention and treatment of diseases and clinical conditions associated with LOC90918. FIG. 1 further provides a conceptual description of a novel bioinformatically detected viral gene of the present invention, referred to here as Viral Genomic Address Messenger 304 (VGAM304) viral gene, which modulates expression of respective host target genes thereof, the function and utility of which host target genes is known in the art.

VGAM304 is a novel bioinformatically detected regulatory, non protein coding, viral micro RNA (miRNA) gene. The method by which VGAM304 was detected is described hereinabove with reference to FIGS. 1-8.

VGAM304 gene, herein designated VGAM GENE, is a viral gene contained in the genome of Vaccinia Virus. VGAM304 host target gene, herein designated VGAM HOST TARGET GENE, is a human gene contained in the human genome.

VGAM304 gene encodes a VGAM304 precursor RNA, herein designated VGAM PRECURSOR RNA. Similar to other miRNA genes, and unlike most ordinary genes, VGAM304 precursor RNA does not encode a protein. A nucleotide sequence identical or highly similar to the nucleotide sequence of VGAM304 precursor RNA is designated SEQ ID:290, and is provided hereinbelow with reference to the sequence listing part. Nucleotide sequence SEQ ID:290 is located at position 179183 relative to the genome of Vaccinia Virus.

VGAM304 precursor RNA folds onto itself, forming VGAM304 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM304 folded precursor RNA into VGAM304 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM304 RNA is designated SEQ ID:639, and is provided hereinbelow with reference to the sequence listing part.

VGAM304 host target gene, herein designated VGAM HOST TARGET GENE, encodes a corresponding messenger RNA, VGAM304 host target RNA, herein designated VGAM HOST TARGET RNA. VGAM304 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM304 RNA, herein designated VGAM RNA, binds complementarily to one or more host target binding sites located in untranslated regions of VGAM304 host target RNA, herein designated VGAM HOST TARGET RNA. This complementary binding is due to the fact that the nucleotide sequence of VGAM304 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM304 RNA, herein designated VGAM RNA, may have a different number of host target binding sites in untranslated regions of a VGAM304 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM304 RNA, herein designated VGAM RNA, to host target binding sites on VGAM304 host target RNA, herein designated VGAM HOST TARGET RNA, such as BINDING SITE I, BINDING SITE II and BINDING SITE III, inhibits translation of VGAM304 host target RNA into VGAM304 host target protein, herein designated VGAM HOST TARGET PROTEIN. VGAM host target protein is therefore outlined by a broken line.

It is appreciated that VGAM304 host target gene, herein designated VGAM HOST TARGET GENE, in fact represents a plurality of VGAM304 host target genes. The mRNA of each one of this plurality of VGAM304 host target genes comprises one or more host target binding sites, each having a nucleotide sequence which is at least partly complementary to VGAM304 RNA, herein designated VGAM RNA, and which when bound by VGAM304 RNA causes inhibition of translation of respective one or more VGAM304 host target proteins.

It is further appreciated by one skilled in the art that the mode of translational inhibition illustrated by FIG. 1 with specific reference to translational inhibition exerted by VGAM304 gene, herein designated VGAM GENE, on one or more VGAM304 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM304 is inhibition of expression of host target genes, as part of a novel viral mechanism of attacking a host. Accordingly, utilities of VGAM304 include diagnosis, prevention and treatment of viral infection by Vaccinia Virus. Specific functions, and accordingly utilities, of VGAM304 correlate with, and may be deduced from, the identity of the host target genes which VGAM304 binds and inhibits, and the function of these host target genes, as elaborated hereinbelow.

Nucleotide sequences of the VGAM304 precursor RNA, herein designated VGAM PRECURSOR RNA, and of the ‘diced’ VGAM304 RNA, herein designated VGAM RNA, and a schematic representation of the secondary folding of VGAM304 folded precursor RNA, herein designated VGAM FOLDED PRECURSOR RNA, of VGAM304 are further described hereinbelow with reference to Table 1.

Nucleotide sequences of host target binding sites, such as BINDING SITE-I, BINDING SITE-II and BINDING SITE-III of FIG. 1, found on VGAM304 host target RNA, and schematic representation of the complementarity of each of these host target binding sites to VGAM304 RNA, herein designated VGAM RNA, are described hereinbelow with reference to Table 2.

As mentioned hereinabove with reference to FIG. 1, a function of VGAM304 gene, herein designated VGAM is inhibition of expression of VGAM304 target genes. It is appreciated that specific functions, and accordingly utilities, of VGAM304 correlate with, and may be deduced from, the identity of the target genes which VGAM304 binds and inhibits, and the function of these target genes, as elaborated hereinbelow.

Platelet-activating Factor Acetylhydrolase, Isoform 1b, Beta Subunit 30 kDa (PAFAH1B2, Accession NM_{—}002572) is a VGAM304 host target gene. PAFAH1B2 BINDING SITE is HOST TARGET binding site found in the 3′ untranslated region of mRNA encoded by PAFAH1B2, corresponding to a HOST TARGET binding site such as BINDING SITE I, BINDING SITE II or BINDING SITE III. Table 2 illustrates the complementarity of the nucleotide sequences of PAFAH1B2 BINDING SITE, designated SEQ ID:937, to the nucleotide sequence of VGAM304 RNA, herein designated VGAM RNA, also designated SEQ ID:639.

A function of VGAM304 is therefore inhibition of Platelet-activating Factor Acetylhydrolase, Isoform 1b, Beta Subunit 30 kDa (PAFAH1B2, Accession NM_{—}002572), a gene which inactivates paf by removing the acetyl group at the sn-2 position. Accordingly, utilities of VGAM304 include diagnosis, prevention and treatment of diseases and clinical conditions associated with PAFAH1B2. The function of PAFAH1B2 has been established by previous studies. Platelet-activating factor (PAF) is a biologically active phospholipid with diverse biologic effects. PAF is degraded to inactive products by hydrolysis of the acetyl group at the sn-2 position to produce the biologically inactive products LYSO-PAF and acetate. This reaction is catalyzed by PAF acetylhydrolase (PAFAH). The various monomeric and multimeric forms of the enzyme are composed of alpha (OMIM Ref. No. 601545), beta, and gamma (OMIM Ref. No. 603074) PAFAH subunits. By screening a human fetal liver library with 2 oligodeoxyribonucleotides derived from the cDNA sequence of the bovine PAFAH beta subunit, Adachi et al. (1997) cloned the cDNA encoding the human PAFAH beta subunit. The PAFAH1B2 gene encodes a 229-amino acid polypeptide with a molecular mass of 30 kD. The human PAFAH1B2 amino acid sequence is 62.4% identical to that of the human gamma subunit. Northern blot analysis revealed that the gene was expressed as a 4.0-kb mRNA in all human adult and fetal tissues tested. By radiation hybrid mapping, screening of a YAC library, and fluorescence in situ hybridization, Moro et al. (1998) localized the PAFAH1B2 gene to 11q23.

Full details of the abovementioned studies are described in the following publications, the disclosure of which are hereby incorporated by reference:
Adachi, H.; Tsujimoto, M.; Hattori, M.; Arai, H.; Inoue, K.: Differential tissue distribution of the beta- and gamma-subunits of human cytosolic platelet-activating factor acetylhydrolase (isoform 1). Biochem. Biophys. Res. Commun. 233: 10-13, 1997.; and
Moro, F.; Arrigo, G.; Fogli, A.; Bernard, L.; Carrozzo, R.: The beta and gamma subunits of the human platelet-activating factor acetyl hydrolase isoform 1b (PAFAH1B2 and PAFAH1B3) map t.

Further studies establishing the function and utilities of PAFAH1B2 are found in John Hopkins OMIM database record ID 602508, and in sited publications numbered 2025-2026 listed in the bibliography section hereinbelow, which are also hereby incorporated by reference. FIG. 1 further provides a conceptual description of a novel bioinformatically detected viral gene of the present invention, referred to here as Viral Genomic Address Messenger 305 (VGAM305) viral gene, which modulates expression of respective host target genes thereof, the function and utility of which host target genes is known in the art.

VGAM305 is a novel bioinformatically detected regulatory, non protein coding, viral micro RNA (miRNA) gene. The method by which VGAM305 was detected is described hereinabove with reference to FIGS. 1-8.

VGAM305 gene, herein designated VGAM GENE, is a viral gene contained in the genome of Vaccinia Virus. VGAM305 host target gene, herein designated VGAM HOST TARGET GENE, is a human gene contained in the human genome.

VGAM305 gene encodes a VGAM305 precursor RNA, herein designated VGAM PRECURSOR RNA. Similar to other miRNA genes, and unlike most ordinary genes, VGAM305 precursor RNA does not encode a protein. A nucleotide sequence identical or highly similar to the nucleotide sequence of VGAM305 precursor RNA is designated SEQ ID:291, and is provided hereinbelow with reference to the sequence listing part. Nucleotide sequence SEQ ID:291 is located at position 179295 relative to the genome of Vaccinia Virus.

VGAM305 precursor RNA folds onto itself, forming VGAM305 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM305 folded precursor RNA into VGAM305 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM305 RNA is designated SEQ ID:640, and is provided hereinbelow with reference to the sequence listing part.

VGAM305 host target gene, herein designated VGAM HOST TARGET GENE, encodes a corresponding messenger RNA, VGAM305 host target RNA, herein designated VGAM HOST TARGET RNA. VGAM305 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM305 RNA, herein designated VGAM RNA, binds complementarily to one or more host target binding sites located in untranslated regions of VGAM305 host target RNA, herein designated VGAM HOST TARGET RNA. This complementary binding is due to the fact that the nucleotide sequence of VGAM305 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM305 RNA, herein designated VGAM RNA, may have a different number of host target binding sites in untranslated regions of a VGAM305 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM305 RNA, herein designated VGAM RNA, to host target binding sites on VGAM305 host target RNA, herein designated VGAM HOST TARGET RNA, such as BINDING SITE I, BINDING SITE II and BINDING SITE III, inhibits translation of VGAM305 host target RNA into VGAM305 host target protein, herein designated VGAM HOST TARGET PROTEIN. VGAM host target protein is therefore outlined by a broken line.

It is appreciated that VGAM305 host target gene, herein designated VGAM HOST TARGET GENE, in fact represents a plurality of VGAM305 host target genes. The mRNA of each one of this plurality of VGAM305 host target genes comprises one or more host target binding sites, each having a nucleotide sequence which is at least partly complementary to VGAM305 RNA, herein designated VGAM RNA, and which when bound by VGAM305 RNA causes inhibition of translation of respective one or more VGAM305 host target proteins.

It is further appreciated by one skilled in the art that the mode of translational inhibition illustrated by FIG. 1 with specific reference to translational inhibition exerted by VGAM305 gene, herein designated VGAM GENE, on one or more VGAM305 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM305 is inhibition of expression of host target genes, as part of a novel viral mechanism of attacking a host. Accordingly, utilities of VGAM305 include diagnosis, prevention and treatment of viral infection by Vaccinia Virus. Specific functions, and accordingly utilities, of VGAM305 correlate with, and may be deduced from, the identity of the host target genes which VGAM305 binds and inhibits, and the function of these host target genes, as elaborated hereinbelow.

Nucleotide sequences of the VGAM305 precursor RNA, herein designated VGAM PRECURSOR RNA, and of the ‘diced’ VGAM305 RNA, herein designated VGAM RNA, and a schematic representation of the secondary folding of VGAM305 folded precursor RNA, herein designated VGAM FOLDED PRECURSOR RNA, of VGAM305 are further described hereinbelow with reference to Table 1.

Nucleotide sequences of host target binding sites, such as BINDING SITE-I, BINDING SITE-II and BINDING SITE-III of FIG. 1, found on VGAM305 host target RNA, and schematic representation of the complementarity of each of these host target binding sites to VGAM305 RNA, herein designated VGAM RNA, are described hereinbelow with reference to Table 2.

As mentioned hereinabove with reference to FIG. 1, a function of VGAM305 gene, herein designated VGAM is inhibition of expression of VGAM305 target genes. It is appreciated that specific functions, and accordingly utilities, of VGAM305 correlate with, and may be deduced from, the identity of the target genes which VGAM305 binds and inhibits, and the function of these target genes, as elaborated hereinbelow.

GL004 (Accession XM_{—}038373) is a VGAM305 host target gene. GL004 BINDING SITE is HOST TARGET binding site found in the 3′ untranslated region of mRNA encoded by GL004, corresponding to a HOST TARGET binding site such as BINDING SITE I, BINDING SITE II or BINDING SITE III. Table 2 illustrates the complementarity of the nucleotide sequences of GL004 BINDING SITE, designated SEQ ID:2736, to the nucleotide sequence of VGAM305 RNA, herein designated VGAM RNA, also designated SEQ ID:640.

A function of VGAM305 is therefore inhibition of GL004 (Accession XM_{—}038373). Accordingly, utilities of VGAM305 include diagnosis, prevention and treatment of diseases and clinical conditions associated with GL004. KIAA1336 (Accession XM_{—}051306) is another VGAM305 host target gene. KIAA1336 BINDING SITE is HOST TARGET binding site found in the 3′ untranslated region of mRNA encoded by KIAA1336, corresponding to a HOST TARGET binding site such as BINDING SITE I, BINDING SITE II or BINDING SITE III. Table 2 illustrates the complementarity of the nucleotide sequences of KIAA1336 BINDING SITE, designated SEQ ID:2942, to the nucleotide sequence of VGAM305 RNA, herein designated VGAM RNA, also designated SEQ ID:640.

Another function of VGAM305 is therefore inhibition of KIAA1336 (Accession XM_{—}051306). Accordingly, utilities of VGAM305 include diagnosis, prevention and treatment of diseases and clinical conditions associated with KIAA1336. LOC147711 (Accession XM_{—}085851) is another VGAM305 host target gene. LOC147711 BINDING SITE is HOST TARGET binding site found in the 3′ untranslated region of mRNA encoded by LOC147711, corresponding to a HOST TARGET binding site such as BINDING SITE I, BINDING SITE II or BINDING SITE III. Table 2 illustrates the complementarity of the nucleotide sequences of LOC147711 BINDING SITE, designated SEQ ID:3112, to the nucleotide sequence of VGAM305 RNA, herein designated VGAM RNA, also designated SEQ ID:640.

Another function of VGAM305 is therefore inhibition of LOC147711 (Accession XM_{—}085851). Accordingly, utilities of VGAM305 include diagnosis, prevention and treatment of diseases and clinical conditions associated with LOC147711. FIG. 1 further provides a conceptual description of a novel bioinformatically detected viral gene of the present invention, referred to here as Viral Genomic Address Messenger 306 (VGAM306) viral gene, which modulates expression of respective host target genes thereof, the function and utility of which host target genes is known in the art.

VGAM306 is a novel bioinformatically detected regulatory, non protein coding, viral micro RNA (miRNA) gene. The method by which VGAM306 was detected is described hereinabove with reference to FIGS. 1-8.

VGAM306 gene, herein designated VGAM GENE, is a viral gene contained in the genome of Vaccinia Virus. VGAM306 host target gene, herein designated VGAM HOST TARGET GENE, is a human gene contained in the human genome.

VGAM306 gene encodes a VGAM306 precursor RNA, herein designated VGAM PRECURSOR RNA. Similar to other miRNA genes, and unlike most ordinary genes, VGAM306 precursor RNA does not encode a protein. A nucleotide sequence identical or highly similar to the nucleotide sequence of VGAM306 precursor RNA is designated SEQ ID:292, and is provided hereinbelow with reference to the sequence listing part. Nucleotide sequence SEQ ID:292 is located at position 176521 relative to the genome of Vaccinia Virus.

VGAM306 precursor RNA folds onto itself, forming VGAM306 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM306 folded precursor RNA into VGAM306 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 73%) nucleotide sequence of VGAM306 RNA is designated SEQ ID:641, and is provided hereinbelow with reference to the sequence listing part.

VGAM306 host target gene, herein designated VGAM HOST TARGET GENE, encodes a corresponding messenger RNA, VGAM306 host target RNA, herein designated VGAM HOST TARGET RNA. VGAM306 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM306 RNA, herein designated VGAM RNA, binds complementarily to one or more host target binding sites located in untranslated regions of VGAM306 host target RNA, herein designated VGAM HOST TARGET RNA. This complementary binding is due to the fact that the nucleotide sequence of VGAM306 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM306 RNA, herein designated VGAM RNA, may have a different number of host target binding sites in untranslated regions of a VGAM306 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM306 RNA, herein designated VGAM RNA, to host target binding sites on VGAM306 host target RNA, herein designated VGAM HOST TARGET RNA, such as BINDING SITE I, BINDING SITE II and BINDING SITE III, inhibits translation of VGAM306 host target RNA into VGAM306 host target protein, herein designated VGAM HOST TARGET PROTEIN. VGAM host target protein is therefore outlined by a broken line.

It is appreciated that VGAM306 host target gene, herein designated VGAM HOST TARGET GENE, in fact represents a plurality of VGAM306 host target genes. The mRNA of each one of this plurality of VGAM306 host target genes comprises one or more host target binding sites, each having a nucleotide sequence which is at least partly complementary to VGAM306 RNA, herein designated VGAM RNA, and which when bound by VGAM306 RNA causes inhibition of translation of respective one or more VGAM306 host target proteins.

It is further appreciated by one skilled in the art that the mode of translational inhibition illustrated by FIG. 1 with specific reference to translational inhibition exerted by VGAM306 gene, herein designated VGAM GENE, on one or more VGAM306 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM306 is inhibition of expression of host target genes, as part of a novel viral mechanism of attacking a host. Accordingly, utilities of VGAM306 include diagnosis, prevention and treatment of viral infection by Vaccinia Virus. Specific functions, and accordingly utilities, of VGAM306 correlate with, and may be deduced from, the identity of the host target genes which VGAM306 binds and inhibits, and the function of these host target genes, as elaborated hereinbelow.

Nucleotide sequences of the VGAM306 precursor RNA, herein designated VGAM PRECURSOR RNA, and of the ‘diced’ VGAM306 RNA, herein designated VGAM RNA, and a schematic representation of the secondary folding of VGAM306 folded precursor RNA, herein designated VGAM FOLDED PRECURSOR RNA, of VGAM306 are further described hereinbelow with reference to Table 1.

Nucleotide sequences of host target binding sites, such as BINDING SITE-I, BINDING SITE-II and BINDING SITE-III of FIG. 1, found on VGAM306 host target RNA, and schematic representation of the complementarity of each of these host target binding sites to VGAM306 RNA, herein designated VGAM RNA, are described hereinbelow with reference to Table 2.

As mentioned hereinabove with reference to FIG. 1, a function of VGAM306 gene, herein designated VGAM is inhibition of expression of VGAM306 target genes. It is appreciated that specific functions, and accordingly utilities, of VGAM306 correlate with, and may be deduced from, the identity of the target genes which VGAM306 binds and inhibits, and the function of these target genes, as elaborated hereinbelow.

UDP-Gal: betaGlcNAc Beta 1,3-galactosyltransferase, Polypeptide 3 (B3GALT3, Accession NM_{—}003781) is a VGAM306 host target gene. B3GALT3 BINDING SITE is HOST TARGET binding site found in the 3′ untranslated region of mRNA encoded by B3GALT3, corresponding to a HOST TARGET binding site such as BINDING SITE I, BINDING SITE II or BINDING SITE III. Table 2 illustrates the complementarity of the nucleotide sequences of B3GALT3 BINDING SITE, designated SEQ ID: 1058, to the nucleotide sequence of VGAM306 RNA, herein designated VGAM RNA, also designated SEQ ID:641.

A function of VGAM306 is therefore inhibition of UDP-Gal:betaGlcNAc Beta 1,3-galactosyltransferase, Polypeptide 3 (B3GALT3, Accession NM_{—}003781). Accordingly, utilities of VGAM306 include diagnosis, prevention and treatment of diseases and clinical conditions associated with B3GALT3. Casein Kinase 1, Gamma 3 (CSNK1G3, Accession NM_{—}004384) is another VGAM306 host target gene. CSNK1G3 BINDING SITE is HOST TARGET binding site found in the 3′ untranslated region of mRNA encoded by CSNK1G3, corresponding to a HOST TARGET binding site such as BINDING SITE I, BINDING SITE II or BINDING SITE III. Table 2 illustrates the complementarity of the nucleotide sequences of CSNK1G3 BINDING SITE, designated SEQ ID: 1106, to the nucleotide sequence of VGAM306 RNA, herein designated VGAM RNA, also designated SEQ ID:641.

Another function of VGAM306 is therefore inhibition of Casein Kinase 1, Gamma 3 (CSNK1G3, Accession NM_{—}004384). Accordingly, utilities of VGAM306 include diagnosis, prevention and treatment of diseases and clinical conditions associated with CSNK1G3. Desmocollin 3 (DSC3, Accession NM_{—}024423) is another VGAM306 host target gene. DSC3 BINDING SITE1 and DSC3 BINDING SITE2 are HOST TARGET binding sites found in untranslated regions of mRNA encoded by DSC3, corresponding to HOST TARGET binding sites such as BINDING SITE I, BINDING SITE II or BINDING SITE III. Table 2 illustrates the complementarity of the nucleotide sequences of DSC3 BINDING SITE1 and DSC3 BINDING SITE2, designated SEQ ID:2061 and SEQ ID:872 respectively, to the nucleotide sequence of VGAM306 RNA, herein designated VGAM RNA, also designated SEQ ID:641.

Another function of VGAM306 is therefore inhibition of Desmocollin 3 (DSC3, Accession NM_{—}024423), a gene which is a component of intercellular desmosome junctions. Accordingly, utilities of VGAM306 include diagnosis, prevention and treatment of diseases and clinical conditions associated with DSC3. The function of DSC3 has been established by previous studies. From a bladder carcinoma cell line cDNA library, Kawamura et al. (1994) cloned a human cDNA encoding for a novel transmembrane protein. Sequence analysis revealed an open reading frame of 2,691 bp encoding a protein of 896 amino acids. Sequence comparisons showed significant homology to desmocollins, intercellular adhesion molecules belonging to the cadherin super family. The protein consisted of a signal peptide of 30 amino acids, a precursor segment of 105 amino acids, and a mature protein of 761 amino acids. Antibodies recognizing the predicted mature adhesion molecule of the protein stained antigens along the cell boundaries of normal human keratinocytes resembling the pattern of desmosome localization. Kawamura et al. (1994) concluded that the clone represented a new member of the desmocollin family and tentatively referred to it as desmocollin type 4. King et al. (1995) used the designation DSC3 for a gene encoding a desmocollin present in human foreskin epidermis and stated that the gene is identical to that encoding the desmocollin isolated from a bladder carcinoma cell line and called DSC4 by Kawamura et al. (1994). They likewise mapped the gene to chromosome 18 by PCR analysis of rodent/human somatic cell hybrids. They stated that the cDNA sequence showed 67% amino acid identity with the original human desmocollin, designated DSC2, and 52% amino acid identity with DSC1. By in situ hybridization studies, they showed that DSC1 was not present in any of the nonkeratinizing human epithelia, such as buccal mucosa, cervix, and esophagus, whereas all these internal epithelia expressed DSC2 and DSC3 and were present in most of the living layers of tissues, including the basal layers.

Full details of the abovementioned studies are described in the following publications, the disclosure of which are hereby incorporated by reference:
Kawamura, K.; Watanabe, K.; Suzuki, T.; Yamakawa, T.; Kamiyama, T.; Nakagawa, H.; Tsurufuji, S.: cDNA cloning and expression of a novel human desmocollin. J. Biol. Chem. 269: 26295-26302, 1994.; and
King, I. A.; Sullivan, K. H.; Bennett, R., Jr.; Buxton, R. S.: The desmocollins of human foreskin epidermis: identification and chromosomal assignment of a third gene and expression p.

Further studies establishing the function and utilities of DSC3 are found in John Hopkins OMIM database record ID 600271, and in sited publications numbered 1659-1661 listed in the bibliography section hereinbelow, which are also hereby incorporated by reference. SUV39H2 (Accession NM_{—}024670) is another VGAM306 host target gene. SUV39H2BINDING SITE is HOST TARGET binding site found in the 3′ untranslated region of mRNA encoded by SUV39H2, corresponding to a HOST TARGET binding site such as BINDING SITE I, BINDING SITE II or BINDING SITE III. Table 2 illustrates the complementarity of the nucleotide sequences of SUV39H2BINDING SITE, designated SEQ ID:2080, to the nucleotide sequence of VGAM306 RNA, herein designated VGAM RNA, also designated SEQ ID:641.

Another function of VGAM306 is therefore inhibition of SUV39H2 (Accession NM_{—}024670), a gene which is involved in gene repression and the modification of position-effect-variegation. Accordingly, utilities of VGAM306 include diagnosis, prevention and treatment of diseases and clinical conditions associated with SUV39H2. The function of SUV39H2 has been established by previous studies. O'Carroll et al. (2000) isolated and characterized a murine gene, Suv39h2, that encodes an H3 histone (see OMIM Ref. No. 601128) methyltransferase (OMIM Ref. No. HMTase) with 59% identity to Suv39h1 (OMIM Ref. No. 300254). Although both Suv39h loci displayed overlapping expression profiles during mouse embryogenesis, Suv39h2 transcripts remained specifically expressed in adult testes. Immunolocalization of the Suv39h2 protein during spermatogenesis indicated enriched distribution at the heterochromatin from the leptotene to the round spermatid stage. Moreover, Suv39h2 specifically accumulated with chromatin of the sex chromosomes (XY body), which undergo transcriptional silencing during the first meiotic prophase. These data were consistent with redundant enzymatic roles for Suv39h1 and Suv39h2 during mouse development and suggested an additional function of the Suv39h2 HMTase in organizing meiotic heterochromatin that may even impart an epigenetic imprint to the male germline. Animal model experiments lend further support to the function of SUV39H2. Peters et al. (2001) generated mice deficient for either Suv39h1 or Suv39h2. These animals displayed normal viability and fertility and did not exhibit apparent phenotypes. The authors subsequently intercrossed Suv39h1 −/− and Suv39h2 −/− mice to generate compound Suv39h mutants that were then used to derive Suv39h double-null mice (Suv39h1 −/− and Suv39h2 −/−). These mice displayed severely impaired viability and chromosomal instabilities that were associated with an increased tumor risk and perturbed chromosome interactions during male meiosis. These data suggested a crucial role for pericentric H3 histone-lys9 methylation in protecting genome stability and defined the Suv39h HM-Tases as important epigenetic regulators for mammalian development.

It is appreciated that the abovementioned animal model for SUV39H2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
O'Carroll, D.; Scherthan, H.; Peters, A. H. F. M.; Opravil, S.; Haynes, A. R.; Laible, G.; Rea, S.; Schmid, M.; Lebersorger, A.; Jerratsch, M.; Sattler, L.; Mattei, M. G.; Denny, P.; Brown, S. D. M.; Schweizer, D.; Jenuwein, T.: Isolation and characterization of Suv39h2, a second histone H3 methyltransferase gene that displays testis-specific expression. Molec. Cell. Biol. 20: 9423-9433, 2000.; and
Peters, A. H. F. M.; O'Carroll, D.; Scherthan, H.; Mechtler, K.; Sauer, S.; Schofer, C.; Weipoltshammer, K.; Pagani, M.; Lachner, M.; Kohlmaier, A.; Opravil, S.; Doyle, M.; Sibilia, M.

Further studies establishing the function and utilities of SUV39H2 are found in John Hopkins OMIM database record ID 606503, and in sited publications numbered 140 and 2031 listed in the bibliography section hereinbelow, which are also hereby incorporated by reference. Tachykinin, Precursor 1 (substance K, substance P, neurokinin 1, neurokinin 2, neuromedin L, neurokinin alpha, neuropeptide K, neuropeptide gamma) (TAC1, Accession NM_{—}013996) is another VGAM306 host target gene. TAC1 BINDING SITE1 through TAC1 BINDING SITE4 are HOST TARGET binding sites found in untranslated regions of mRNA encoded by TAC1, corresponding to HOST TARGET binding sites such as BINDING SITE I, BINDING SITE II or BINDING SITE III. Table 2 illustrates the complementarity of the nucleotide sequences of TAC1 BINDING SITE1 through TAC1 BINDING SITE4, designated SEQ ID:1456, SEQ ID:1457, SEQ ID:1458 and SEQ ID:996 respectively, to the nucleotide sequence of VGAM306 RNA, herein designated VGAM RNA, also designated SEQ ID:641.

Another function of VGAM306 is therefore inhibition of Tachykinin, Precursor 1 (substance K, substance P, neurokinin 1, neurokinin 2, neuromedin L, neurokinin alpha, neuropeptide K, neuropeptide gamma) (TAC1, Accession NM_{—}013996), a gene which is precursor of tachykinin-A (substance-K, neurokinin A) and substance-P, which modulate immune and nerve cells. Accordingly, utilities of VGAM306 include diagnosis, prevention and treatment of diseases and clinical conditions associated with TAC1. The function of TAC1 has been established by previous studies. The tachykinins are a family of amidated neuropeptides that share a carboxy-terminal sequence PheX-Gly-Leu-Met-NH2 and are found in both vertebrates and invertebrates. The 3 known tachykinins in man are encoded by 2 genes. One gene encodes a precursor containing both substance P and neurokinin A, while the other encodes a precursor containing only neurokinin B (OMIM Ref. No. 162330). (Neurokinin A was formerly known as substance K.) Using probes derived from the cloned human genes and a panel of rodent-human somatic cell hybrids, Bonner et al. (1987) assigned the NKNA gene to 7q21-q22 and the NKNB gene to 12q13-q21. Molecular characterization of the tachykinins show that they arise from common precursor molecules known as preprotachykinins by proteolytic processing. Three forms of message (alpha, beta, and gamma) arise by alternative splicing events (Krause et al., 1987). The beta and gamma forms of preprotachykinins encode both substance P and neurokinin A, while the alpha form contains only the substance P sequence. Bahary et al. (1991) mapped the homologous gene to mouse chromosome 6. Cao et al. (1998) disrupted the mouse preprotachykinin A gene, which encodes both substance P and neurokinin A. The behavioral response to mildly painful stimuli was intact in mutant mice; however, the response to moderate to intense pain was significantly reduced. Neurogenic inflammation, which results from peripheral release of substance P and neurokinin A, is almost absent in the mutant mice. The contribution of substance P and neurokinin A is neither modality-nor tissue-specific; pain behaviors evoked by thermal, mechanical, and chemical stimulation of somatic and visceral tissues were all reduced in the mutant mice. The authors concluded that the release of tachykinins from primary afferent pain-sensing receptors (nociceptors) is required to produce moderate to intense pain. Cao et al. (1998) proposed that glutamate is the neurotransmitter for basal pain response, that substance P and neurokinin A are released with higher stimuli, and that at the highest intensities, an undefined system comes into play.

Full details of the abovementioned studies are described in the following publications, the disclosure of which are hereby incorporated by reference:
Bahary, N.; Zorich, G.; Pachter, J. E.; Leibel, R. L.; Friedman, J. M.: Molecular genetic linkage maps of mouse chromosomes 4 and 6. Genomics 11: 33-47, 1991.; and
Cao, Y. Q.; Mantyh, P. W.; Carlson, E. J.; Gillespie, A.-M.; Epstein, C. J.; Basbaum, A. I.: Primary afferent tachykinins are required to experience moderate to intense pain. Nature 392.

Further studies establishing the function and utilities of TAC1 are found in John Hopkins OMIM database record ID 162320, and in sited publications numbered 111 and 2542-2547 listed in the bibliography section hereinbelow, which are also hereby incorporated by reference. Zinc Finger Protein 207 (ZNF207, Accession NM_{—}003457) is another VGAM306 host target gene. ZNF207 BINDING SITE is HOST TARGET binding site found in the 3′ untranslated region of mRNA encoded by ZNF207, corresponding to a HOST TARGET binding site such as BINDING SITE I, BINDING SITE II or BINDING SITE III. Table 2 illustrates the complementarity of the nucleotide sequences of ZNF207 BINDING SITE, designated SEQ ID: 1023, to the nucleotide sequence of VGAM306 RNA, herein designated VGAM RNA, also designated SEQ ID:641.

Another function of VGAM306 is therefore inhibition of Zinc Finger Protein 207 (ZNF207, Accession NM_{—}003457). Accordingly, utilities of VGAM306 include diagnosis, prevention and treatment of diseases and clinical conditions associated with ZNF207. A Kinase (PRKA) Anchor Protein (gravin) 12 (AKAP12, Accession NM_{—}005100) is another VGAM306 host target gene. AKAP12 BINDING SITE1 and AKAP12 BINDING SITE2 are HOST TARGET binding sites found in untranslated regions of mRNA encoded by AKAP12, corresponding to HOST TARGET binding sites such as BINDING SITE I, BINDING SITE II or BINDING SITE III. Table 2 illustrates the complementarity of the nucleotide sequences of AKAP12 BINDING SITE1 and AKAP12 BINDING SITE2, designated SEQ ID:1185 and SEQ ID:2481 respectively, to the nucleotide sequence of VGAM306 RNA, herein designated VGAM RNA, also designated SEQ ID:641.

Another function of VGAM306 is therefore inhibition of A Kinase (PRKA) Anchor Protein (gravin) 12 (AKAP12, Accession NM_{—}005100). Accordingly, utilities of VGAM306 include diagnosis, prevention and treatment of diseases and clinical conditions associated with AKAP12. FLJ11222 (Accession NM_{—}018365) is another VGAM306 host target gene. FLJ11222 BINDING SITE is HOST TARGET binding site found in the 3′ untranslated region of mRNA encoded by FLJ11222, corresponding to a HOST TARGET binding site such as BINDING SITE I, BINDING SITE II or BINDING SITE III. Table 2 illustrates the complementarity of the nucleotide sequences of FLJ11222 BINDING SITE, designated SEQ ID: 1819, to the nucleotide sequence of VGAM306 RNA, herein designated VGAM RNA, also designated SEQ ID:641.

Another function of VGAM306 is therefore inhibition of FLJ11222 (Accession NM_{—}018365). Accordingly, utilities of VGAM306 include diagnosis, prevention and treatment of diseases and clinical conditions associated with FLJ11222. FLJ14641 (Accession NM_{—}032817) is another VGAM306 host target gene. FLJ14641 BINDING SITE is HOST TARGET binding site found in the 3′ untranslated region of mRNA encoded by FLJ14641, corresponding to a HOST TARGET binding site such as BINDING SITE I, BINDING SITE II or BINDING SITE III. Table 2 illustrates the complementarity of the nucleotide sequences of FLJ14641 BINDING SITE, designated SEQ ID:2284, to the nucleotide sequence of VGAM306 RNA, herein designated VGAM RNA, also designated SEQ ID:641.

Another function of VGAM306 is therefore inhibition of FLJ14641 (Accession NM_{—}032817). Accordingly, utilities of VGAM306 include diagnosis, prevention and treatment of diseases and clinical conditions associated with FLJ14641. FLJ23537 (Accession NM_{—}024889) is another VGAM306 host target gene. FLJ23537 BINDING SITE is HOST TARGET binding site found in the 3′ untranslated region of mRNA encoded by FLJ23537, corresponding to a HOST TARGET binding site such as BINDING SITE I, BINDING SITE II or BINDING SITE III. Table 2 illustrates the complementarity of the nucleotide sequences of FLJ23537 BINDING SITE, designated SEQ ID:2105, to the nucleotide sequence of VGAM306 RNA, herein designated VGAM RNA, also designated SEQ ID:641.

Another function of VGAM306 is therefore inhibition of FLJ23537 (Accession NM_{—}024889). Accordingly, utilities of VGAM306 include diagnosis, prevention and treatment of diseases and clinical conditions associated with FLJ23537. KIAA1161 (Accession XM_{—}088501) is another VGAM306 host target gene. KIAA1161 BINDING SITE is HOST TARGET binding site found in the 5′ untranslated region of mRNA encoded by KIAA1161, corresponding to a HOST TARGET binding site such as BINDING SITE I, BINDING SITE II or BINDING SITE III. Table 2 illustrates the complementarity of the nucleotide sequences of KIAA1161 BINDING SITE, designated SEQ ID:3212, to the nucleotide sequence of VGAM306 RNA, herein designated VGAM RNA, also designated SEQ ID:641.

Another function of VGAM306 is therefore inhibition of KIAA1161 (Accession XM_{—}088501). Accordingly, utilities of VGAM306 include diagnosis, prevention and treatment of diseases and clinical conditions associated with KIAA1161. MGC10646 (Accession NM_{—}032693) is another VGAM306 host target gene. MGC10646 BINDING SITE is HOST TARGET binding site found in the 3′ untranslated region of mRNA encoded by MGC10646, corresponding to a HOST TARGET binding site such as BINDING SITE I, BINDING SITE II or BINDING SITE III. Table 2 illustrates the complementarity of the nucleotide sequences of MGC10646 BINDING SITE, designated SEQ ID:2270, to the nucleotide sequence of VGAM306 RNA, herein designated VGAM RNA, also designated SEQ ID:641.

Another function of VGAM306 is therefore inhibition of MGC10646 (Accession NM_{—}032693). Accordingly, utilities of VGAM306 include diagnosis, prevention and treatment of diseases and clinical conditions associated with MGC10646. Myeloid/lymphoid Or Mixed-lineage Leukemia 5 (trithorax homolog, Drosophila) (MLL5, Accession XM_{—}168551) is another VGAM306 host target gene. MLL5 BINDING SITE is HOST TARGET binding site found in the 3′ untranslated region of mRNA encoded by MLL5, corresponding to a HOST TARGET binding site such as BINDING SITE I, BINDING SITE II or BINDING SITE III. Table 2 illustrates the complementarity of the nucleotide sequences of MLL5 BINDING SITE, designated SEQ ID:3644, to the nucleotide sequence of VGAM306 RNA, herein designated VGAM RNA, also designated SEQ ID:641.

Another function of VGAM306 is therefore inhibition of Myeloid/lymphoid Or Mixed-lineage Leukemia 5 (trithorax homolog, Drosophila) (MLL5, Accession XM_{—}168551). Accordingly, utilities of VGAM306 include diagnosis, prevention and treatment of diseases and clinical conditions associated with MLL5. ZAK (Accession NM_{—}133646) is another VGAM306 host target gene. ZAK BINDING SITE is HOST TARGET binding site found in the 3′ untranslated region of mRNA encoded by ZAK, corresponding to a HOST TARGET binding site such as BINDING SITE I, BINDING SITE II or BINDING SITE III. Table 2 illustrates the complementarity of the nucleotide sequences of ZAK BINDING SITE, designated SEQ ID:2426, to the nucleotide sequence of VGAM306 RNA, herein designated VGAM RNA, also designated SEQ ID:641.

Another function of VGAM306 is therefore inhibition of ZAK (Accession NM_{—}133646). Accordingly, utilities of VGAM306 include diagnosis, prevention and treatment of diseases and clinical conditions associated with ZAK. Zinc Finger Protein 387 (ZNF387, Accession NM_{—}014682) is another VGAM306 host target gene. ZNF387 BINDING SITE is HOST TARGET binding site found in the 3′ untranslated region of mRNA encoded by ZNF387, corresponding to a HOST TARGET binding site such as BINDING SITE I, BINDING SITE II or BINDING SITE III. Table 2 illustrates the complementarity of the nucleotide sequences of ZNF387 BINDING SITE, designated SEQ ID:1520, to the nucleotide sequence of VGAM306 RNA, herein designated VGAM RNA, also designated SEQ ID:641.

Another function of VGAM306 is therefore inhibition of Zinc Finger Protein 387 (ZNF387, Accession NM_{—}014682). Accordingly, utilities of VGAM306 include diagnosis, prevention and treatment of diseases and clinical conditions associated with ZNF387. LOC126661 (Accession XM_{—}059061) is another VGAM306 host target gene. LOC126661 BINDING SITE is HOST TARGET binding site found in the 3′ untranslated region of mRNA encoded by LOC126661, corresponding to a HOST TARGET binding site such as BINDING SITE I, BINDING SITE II or BINDING SITE III. Table 2 illustrates the complementarity of the nucleotide sequences of LOC126661 BINDING SITE, designated SEQ ID:3000, to the nucleotide sequence of VGAM306 RNA, herein designated VGAM RNA, also designated SEQ ID:641.

Another function of VGAM306 is therefore inhibition of LOC126661 (Accession XM_{—}059061). Accordingly, utilities of VGAM306 include diagnosis, prevention and treatment of diseases and clinical conditions associated with LOC126661. LOC139770 (Accession XM_{—}060053) is another VGAM306 host target gene. LOC139770 BINDING SITE is HOST TARGET binding site found in the 3′ untranslated region of mRNA encoded by LOC139770, corresponding to a HOST TARGET binding site such as BINDING SITE I, BINDING SITE II or BINDING SITE III. Table 2 illustrates the complementarity of the nucleotide sequences of LOC139770 BINDING SITE, designated SEQ ID:3022, to the nucleotide sequence of VGAM306 RNA, herein designated VGAM RNA, also designated SEQ ID:641.

Another function of VGAM306 is therefore inhibition of LOC139770 (Accession XM_{—}060053). Accordingly, utilities of VGAM306 include diagnosis, prevention and treatment of diseases and clinical conditions associated with LOC139770. LOC150577 (Accession XM_{—}097918) is another VGAM306 host target gene. LOC150577 BINDING SITE is HOST TARGET binding site found in the 3′ untranslated region of mRNA encoded by LOC150577, corresponding to a HOST TARGET binding site such as BINDING SITE I, BINDING SITE II or BINDING SITE III. Table 2 illustrates the complementarity of the nucleotide sequences of LOC150577 BINDING SITE, designated SEQ ID:3329, to the nucleotide sequence of VGAM306 RNA, herein designated VGAM RNA, also designated SEQ ID:641.

Another function of VGAM306 is therefore inhibition of LOC150577 (Accession XM_{—}097918). Accordingly, utilities of VGAM306 include diagnosis, prevention and treatment of diseases and clinical conditions associated with LOC150577. LOC89919 (Accession XM_{—}027244) is another VGAM306 host target gene. LOC89919 BINDING SITE is HOST TARGET binding site found in the 5′ untranslated region of mRNA encoded by LOC89919, corresponding to a HOST TARGET binding site such as BINDING SITE I, BINDING SITE II or BINDING SITE III. Table 2 illustrates the complementarity of the nucleotide sequences of LOC89919 BINDING SITE, designated SEQ ID:2579, to the nucleotide sequence of VGAM306 RNA, herein designated VGAM RNA, also designated SEQ ID:641.

Another function of VGAM306 is therefore inhibition of LOC89919 (Accession XM_{—}027244). Accordingly, utilities of VGAM306 include diagnosis, prevention and treatment of diseases and clinical conditions associated with LOC89919. LOC92573 (Accession XM_{—}045884) is another VGAM306 host target gene. LOC92573 BINDING SITE is HOST TARGET binding site found in the 5′ untranslated region of mRNA encoded by LOC92573, corresponding to a HOST TARGET binding site such as BINDING SITE I, BINDING SITE II or BINDING SITE III. Table 2 illustrates the complementarity of the nucleotide sequences of LOC92573 BINDING SITE, designated SEQ ID:2869, to the nucleotide sequence of VGAM306 RNA, herein designated VGAM RNA, also designated SEQ ID:641.

Another function of VGAM306 is therefore inhibition of LOC92573 (Accession XM_{—}045884). Accordingly, utilities of VGAM306 include diagnosis, prevention and treatment of diseases and clinical conditions associated with LOC92573. FIG. 1 further provides a conceptual description of a novel bioinformatically detected viral gene of the present invention, referred to here as Viral Genomic Address Messenger 307 (VGAM307) viral gene, which modulates expression of respective host target genes thereof, the function and utility of which host target genes is known in the art.

VGAM307 is a novel bioinformatically detected regulatory, non protein coding, viral micro RNA (miRNA) gene. The method by which VGAM307 was detected is described hereinabove with reference to FIGS. 1-8.

VGAM307 gene, herein designated VGAM GENE, is a viral gene contained in the genome of Vaccinia Virus. VGAM307 host target gene, herein designated VGAM HOST TARGET GENE, is a human gene contained in the human genome.

VGAM307 gene encodes a VGAM307 precursor RNA, herein designated VGAM PRECURSOR RNA. Similar to other miRNA genes, and unlike most ordinary genes, VGAM307 precursor RNA does not encode a protein. A nucleotide sequence identical or highly similar to the nucleotide sequence of VGAM307 precursor RNA is designated SEQ ID:293, and is provided hereinbelow with reference to the sequence listing part. Nucleotide sequence SEQ ID:293 is located at position 10139 relative to the genome of Vaccinia Virus.

VGAM307 precursor RNA folds onto itself, forming VGAM307 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM307 folded precursor RNA into VGAM307 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM307 RNA is designated SEQ ID:642, and is provided hereinbelow with reference to the sequence listing part.

VGAM307 host target gene, herein designated VGAM HOST TARGET GENE, encodes a corresponding messenger RNA, VGAM307 host target RNA, herein designated VGAM HOST TARGET RNA. VGAM307 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM307 RNA, herein designated VGAM RNA, binds complementarily to one or more host target binding sites located in untranslated regions of VGAM307 host target RNA, herein designated VGAM HOST TARGET RNA. This complementary binding is due to the fact that the nucleotide sequence of VGAM307 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM307 RNA, herein designated VGAM RNA, may have a different number of host target binding sites in untranslated regions of a VGAM307 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM307 RNA, herein designated VGAM RNA, to host target binding sites on VGAM307 host target RNA, herein designated VGAM HOST TARGET RNA, such as BINDING SITE I, BINDING SITE II and BINDING SITE III, inhibits translation of VGAM307 host target RNA into VGAM307 host target protein, herein designated VGAM HOST TARGET PROTEIN. VGAM host target protein is therefore outlined by a broken line.

It is appreciated that VGAM307 host target gene, herein designated VGAM HOST TARGET GENE, in fact represents a plurality of VGAM307 host target genes. The mRNA of each one of this plurality of VGAM307 host target genes comprises one or more host target binding sites, each having a nucleotide sequence which is at least partly complementary to VGAM307 RNA, herein designated VGAM RNA, and which when bound by VGAM307 RNA causes inhibition of translation of respective one or more VGAM307 host target proteins.

It is further appreciated by one skilled in the art that the mode of translational inhibition illustrated by FIG. 1 with specific reference to translational inhibition exerted by VGAM307 gene, herein designated VGAM GENE, on one or more VGAM307 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM307 is inhibition of expression of host target genes, as part of a novel viral mechanism of attacking a host. Accordingly, utilities of VGAM307 include diagnosis, prevention and treatment of viral infection by Vaccinia Virus. Specific functions, and accordingly utilities, of VGAM307 correlate with, and may be deduced from, the identity of the host target genes which VGAM307 binds and inhibits, and the function of these host target genes, as elaborated hereinbelow.

Nucleotide sequences of the VGAM307 precursor RNA, herein designated VGAM PRECURSOR RNA, and of the ‘diced’ VGAM307 RNA, herein designated VGAM RNA, and a schematic representation of the secondary folding of VGAM307 folded precursor RNA, herein designated VGAM FOLDED PRECURSOR RNA, of VGAM307 are further described hereinbelow with reference to Table 1.

Nucleotide sequences of host target binding sites, such as BINDING SITE-I, BINDING SITE-II and BINDING SITE-III of FIG. 1, found on VGAM307 host target RNA, and schematic representation of the complementarity of each of these host target binding sites to VGAM307 RNA, herein designated VGAM RNA, are described hereinbelow with reference to Table 2.

As mentioned hereinabove with reference to FIG. 1, a function of VGAM307 gene, herein designated VGAM is inhibition of expression of VGAM307 target genes. It is appreciated that specific functions, and accordingly utilities, of VGAM307 correlate with, and may be deduced from, the identity of the target genes which VGAM307 binds and inhibits, and the function of these target genes, as elaborated hereinbelow.

Neurotrophic Tyrosine Kinase, Receptor, Type 2 (NTRK2, Accession NM_{—}006180) is a VGAM307 host target gene. NTRK2 BINDING SITE is HOST TARGET binding site found in the 3′ untranslated region of mRNA encoded by NTRK2, corresponding to a HOST TARGET binding site such as BINDING SITE I, BINDING SITE II or BINDING SITE III. Table 2 illustrates the complementarity of the nucleotide sequences of NTRK2 BINDING SITE, designated SEQ ID:1275, to the nucleotide sequence of VGAM307 RNA, herein designated VGAM RNA, also designated SEQ ID:642.

A function of VGAM307 is therefore inhibition of Neurotrophic Tyrosine Kinase, Receptor, Type 2 (NTRK2, Accession NM_{—}006180), a gene which is involved in the development and/or maintenance of the nervous system. Accordingly, utilities of VGAM307 include diagnosis, prevention and treatment of diseases and clinical conditions associated with NTRK2. The function of NTRK2 has been established by previous studies. Nakagawara et al. (1995) isolated cDNAs spanning the entire coding region of both human full-length and truncated forms of TRKB from human brain cDNA libraries. The full-length TRKB coded for a protein of 822 amino acid residues. The putative mature peptide sequence was 49% and 55% homologous to human NTRK1 and NTRK3, respectively. Nine of 13 cysteine residues, 4 of 12 N-glycosylation sites in the extracellular domain, and 10 of 13 tyrosine residues in the intracellular domain are conserved among NTRK1, NTRK2, and NTRK3. Two major sizes of NTRK2 transcripts were expressed in human brain. Animal model experiments lend further support to the function of NTRK2. To study the function of TRKB in the cerebellum, Rico et al. (2002) deleted the Trkb gene in mouse cerebellar precursors by Wnt1-driven Cre-mediated recombination. Despite the absence of Trkb, the mature cerebellum of mutant mice appeared similar to that of wildtype, with all types of cells present in normal numbers and positions. Granule and Purkinje cell dendrites appeared normal, and the former had typical numbers of excitatory synapses. By contrast, inhibitory interneurons were strongly affected. Although present in normal number, inhibitory interneurons exhibited reduced amounts of GABAergic markers and developed reduced numbers of GABAergic boutons and synaptic specializations. Thus, Rico et al. (2002) concluded that TRKB is essential to the development of GABAergic neurons and regulates synapse formation in addition to its role in the development of axon terminals.

It is appreciated that the abovementioned animal model for NTRK2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Nakagawara, A.; Liu, X.-G.; Ikegaki, N.; White, P. S.; Yamashiro, D. J.; Nycum, L. M.; Biegel, J. A.; Brodeur, G. M.: Cloning and chromosomal localization of the human TRKB tyrosine kinase receptor gene (NTRK2). Genomics 25: 538-546, 1995.; and
Rico, B.; Xu, B.; Reichardt, L. F.: TrkB receptor signaling is required for establishment of GABAergic synapses in the cerebellum. Nature Neurosci. 5: 225-233, 2002.

Further studies establishing the function and utilities of NTRK2 are found in John Hopkins OMIM database record ID 600456, and in sited publications numbered 407-409, 2972-35 and 2957 listed in the bibliography section hereinbelow, which are also hereby incorporated by reference. Inhibitor of Growth Family, Member 3 (ING3, Accession NM_{—}019071) is another VGAM307 host target gene. ING3 BINDING SITE is HOST TARGET binding site found in the 3′ untranslated region of mRNA encoded by ING3, corresponding to a HOST TARGET binding site such as BINDING SITE I, BINDING SITE II or BINDING SITE III. Table 2 illustrates the complementarity of the nucleotide sequences of ING3 BINDING SITE, designated SEQ ID:1879, to the nucleotide sequence of VGAM307 RNA, herein designated VGAM RNA, also designated SEQ ID:642.

Another function of VGAM307 is therefore inhibition of Inhibitor of Growth Family, Member 3 (ING3, Accession NM_{—}019071). Accordingly, utilities of VGAM307 include diagnosis, prevention and treatment of diseases and clinical conditions associated with ING3. KIAA1164 (Accession XM_{—}045358) is another VGAM307 host target gene. KIAA1164 BINDING SITE is HOST TARGET binding site found in the 5′ untranslated region of mRNA encoded by KIAA1164, corresponding to a HOST TARGET binding site such as BINDING SITE I, BINDING SITE II or BINDING SITE III. Table 2 illustrates the complementarity of the nucleotide sequences of KIAA1164 BINDING SITE, designated SEQ ID:2861, to the nucleotide sequence of VGAM307 RNA, herein designated VGAM RNA, also designated SEQ ID:642.

Another function of VGAM307 is therefore inhibition of KIAA1164 (Accession XM_{—}045358). Accordingly, utilities of VGAM307 include diagnosis, prevention and treatment of diseases and clinical conditions associated with KIAA1164. LOC150577 (Accession XM_{—}097918) is another VGAM307 host target gene. LOC150577 BINDING SITE is HOST TARGET binding site found in the 5′ untranslated region of mRNA encoded by LOC150577, corresponding to a HOST TARGET binding site such as BINDING SITE I, BINDING SITE II or BINDING SITE III. Table 2 illustrates the complementarity of the nucleotide sequences of LOC150577 BINDING SITE, designated SEQ ID:3328, to the nucleotide sequence of VGAM307 RNA, herein designated VGAM RNA, also designated SEQ ID:642.

Another function of VGAM307 is therefore inhibition of LOC150577 (Accession XM_{—}097918). Accordingly, utilities of VGAM307 include diagnosis, prevention and treatment of diseases and clinical conditions associated with LOC150577. LOC199936 (Accession XM_{—}117158) is another VGAM307 host target gene. LOC199936 BINDING SITE is HOST TARGET binding site found in the 3′ untranslated region of mRNA encoded by LOC199936, corresponding to a HOST TARGET binding site such as BINDING SITE I, BINDING SITE II or BINDING SITE III. Table 2 illustrates the complementarity of the nucleotide sequences of LOC199936 BINDING SITE, designated SEQ ID:3474, to the nucleotide sequence of VGAM307 RNA, herein designated VGAM RNA, also designated SEQ ID:642.

Another function of VGAM307 is therefore inhibition of LOC199936 (Accession XM_{—}117158). Accordingly, utilities of VGAM307 include diagnosis, prevention and treatment of diseases and clinical conditions associated with LOC199936. LOC257235 (Accession XM_{—}173124) is another VGAM307 host target gene. LOC257235 BINDING SITE is HOST TARGET binding site found in the 3′ untranslated region of mRNA encoded by LOC257235, corresponding to a HOST TARGET binding site such as BINDING SITE I, BINDING SITE II or BINDING SITE III. Table 2 illustrates the complementarity of the nucleotide sequences of LOC257235 BINDING SITE, designated SEQ ID:3727, to the nucleotide sequence of VGAM307 RNA, herein designated VGAM RNA, also designated SEQ ID:642.

Another function of VGAM307 is therefore inhibition of LOC257235 (Accession XM_{—}173124). Accordingly, utilities of VGAM307 include diagnosis, prevention and treatment of diseases and clinical conditions associated with LOC257235. FIG. 1 further provides a conceptual description of a novel bioinformatically detected viral gene of the present invention, referred to here as Viral Genomic Address Messenger 308 (VGAM308) viral gene, which modulates expression of respective host target genes thereof, the function and utility of which host target genes is known in the art.

VGAM308 is a novel bioinformatically detected regulatory, non protein coding, viral micro RNA (miRNA) gene. The method by which VGAM308 was detected is described hereinabove with reference to FIGS. 1-8.

VGAM308 gene, herein designated VGAM GENE, is a viral gene contained in the genome of Vaccinia Virus. VGAM308 host target gene, herein designated VGAM HOST TARGET GENE, is a human gene contained in the human genome.

VGAM308 gene encodes a VGAM308 precursor RNA, herein designated VGAM PRECURSOR RNA. Similar to other miRNA genes, and unlike most ordinary genes, VGAM308 precursor RNA does not encode a protein. A nucleotide sequence identical or highly similar to the nucleotide sequence of VGAM308 precursor RNA is designated SEQ ID:294, and is provided hereinbelow with reference to the sequence listing part. Nucleotide sequence SEQ ID:294 is located at position 9119 relative to the genome of Vaccinia Virus.

VGAM308 precursor RNA folds onto itself, forming VGAM308 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM308 folded precursor RNA into VGAM308 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM308 RNA is designated SEQ ID:643, and is provided hereinbelow with reference to the sequence listing part.

VGAM308 host target gene, herein designated VGAM HOST TARGET GENE, encodes a corresponding messenger RNA, VGAM308 host target RNA, herein designated VGAM HOST TARGET RNA. VGAM308 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM308 RNA, herein designated VGAM RNA, binds complementarily to one or more host target binding sites located in untranslated regions of VGAM308 host target RNA, herein designated VGAM HOST TARGET RNA. This complementary binding is due to the fact that the nucleotide sequence of VGAM308 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM308 RNA, herein designated VGAM RNA, may have a different number of host target binding sites in untranslated regions of a VGAM308 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM308 RNA, herein designated VGAM RNA, to host target binding sites on VGAM308 host target RNA, herein designated VGAM HOST TARGET RNA, such as BINDING SITE I, BINDING SITE II and BINDING SITE III, inhibits translation of VGAM308 host target RNA into VGAM308 host target protein, herein designated VGAM HOST TARGET PROTEIN. VGAM host target protein is therefore outlined by a broken line.

It is appreciated that VGAM308 host target gene, herein designated VGAM HOST TARGET GENE, in fact represents a plurality of VGAM308 host target genes. The mRNA of each one of this plurality of VGAM308 host target genes comprises one or more host target binding sites, each having a nucleotide sequence which is at least partly complementary to VGAM308 RNA, herein designated VGAM RNA, and which when bound by VGAM308 RNA causes inhibition of translation of respective one or more VGAM308 host target proteins.

It is further appreciated by one skilled in the art that the mode of translational inhibition illustrated by FIG. 1 with specific reference to translational inhibition exerted by VGAM308 gene, herein designated VGAM GENE, on one or more VGAM308 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM308 is inhibition of expression of host target genes, as part of a novel viral mechanism of attacking a host. Accordingly, utilities of VGAM308 include diagnosis, prevention and treatment of viral infection by Vaccinia Virus. Specific functions, and accordingly utilities, of VGAM308 correlate with, and may be deduced from, the identity of the host target genes which VGAM308 binds and inhibits, and the function of these host target genes, as elaborated hereinbelow.

Nucleotide sequences of the VGAM308 precursor RNA, herein designated VGAM PRECURSOR RNA, and of the ‘diced’ VGAM308 RNA, herein designated VGAM RNA, and a schematic representation of the secondary folding of VGAM308 folded precursor RNA, herein designated VGAM FOLDED PRECURSOR RNA, of VGAM308 are further described hereinbelow with reference to Table 1.

Nucleotide sequences of host target binding sites, such as BINDING SITE-I, BINDING SITE-II and BINDING SITE-III of FIG. 1, found on VGAM308 host target RNA, and schematic representation of the complementarity of each of these host target binding sites to VGAM308 RNA, herein designated VGAM RNA, are described hereinbelow with reference to Table 2.

As mentioned hereinabove with reference to FIG. 1, a function of VGAM308 gene, herein designated VGAM is inhibition of expression of VGAM308 target genes. It is appreciated that specific functions, and accordingly utilities, of VGAM308 correlate with, and may be deduced from, the identity of the target genes which VGAM308 binds and inhibits, and the function of these target genes, as elaborated hereinbelow.

Collagen, Type XIX, Alpha 1 (COL19A1, Accession NM_{—}001858) is a VGAM308 host target gene. COL19A1 BINDING SITE is HOST TARGET binding site found in the 3′ untranslated region of mRNA encoded by COL19A1, corresponding to a HOST TARGET binding site such as BINDING SITE I, BINDING SITE II or BINDING SITE III. Table 2 illustrates the complementarity of the nucleotide sequences of COL19A1 BINDING SITE, designated SEQ ID:863, to the nucleotide sequence of VGAM308 RNA, herein designated VGAM RNA, also designated SEQ ID:643.

A function of VGAM308 is therefore inhibition of Collagen, Type XIX, Alpha 1 (COL19A1, Accession NM_{—}001858), a gene which may act as a cross-bridge between fibrils and other extracellular matrix molecules. Accordingly, utilities of VGAM308 include diagnosis, prevention and treatment of diseases and clinical conditions associated with COL19A1. The function of COL19A1 has been established by previous studies. The collagens are a large super family of genes that include a number of subgroups. One such group is composed of fibrillar associated collagens with interrupted triple helices (FACIT) and includes collagen types IX (e.g., 120210), XII (e.g., 120320), XIV (e.g., 120324), and XVI (e.g., 120326). Members of this group have common structural features, including short stretches of collagenous domains interrupted by noncollagenous regions. These, in turn, form functional units that serve to produce adhesion to the fibrils, provide a rigid arm that projects from the fibril and provide a point of interaction with other matrix components Yoshioka et al. (1992) mapped the COL19A1 gene to 6q12-q14, where the COL9A1 gene (OMIM Ref. No. 120210) has been mapped. Myers et al. (1993) mapped the COL19A1 gene to chromosome 6 by analysis of a panel of somatic cell hybrids. By FISH, Gerecke et al. (1997) mapped the COL19A1 gene to 6q12-q13. Khaleduzzaman et al. (1997) showed that the mouse Col19a1 gene is located on chromosome 1A3, where Col9a1 had also been mapped. They suggested that COL19A1 and COL9A1, and their murine counterparts, were duplicated from the same ancestral gene of the FACIT family

Full details of the abovementioned studies are described in the following publications, the disclosure of which are hereby incorporated by reference:
Yoshioka, H.; Zhang, H.; Ramirez, F.; Mattei, M.-G.; Moradi-Ameli, M.; van der Rest, M.; Gordon, M. K.: Synteny between the loci for a novel FACIT-like collagen (D6S228E) and alpha 1(IX) collagen (COL9A1) on 6q12-q14 in humans. Genomics 13: 884-886, 1992.; and
Yoshioka, H.; Zhang, H.; Ramirez, F.; Mattei, M.-G.; Moradi-Ameli, M.; van der Rest, M.; Gordon, M. K.: Synteny between the loci for a novel FACIT-like collagen (D6S228E) and alpha 1(IX.

Further studies establishing the function and utilities of COL19A1 are found in John Hopkins OMIM database record ID 120165, and in sited publications numbered 841-840 listed in the bibliography section hereinbelow, which are also hereby incorporated by reference. EGF-containing Fibulin-like Extracellular Matrix Protein 1 (EFEMP1, Accession NM_{—}004105) is another VGAM308 host target gene. EFEMP1 BINDING SITE1 and EFEMP1 BINDING SITE2 are HOST TARGET binding sites found in untranslated regions of mRNA encoded by EFEMP1, corresponding to HOST TARGET binding sites such as BINDING SITE I, BINDING SITE II or BINDING SITE III. Table 2 illustrates the complementarity of the nucleotide sequences of EFEMP1 BINDING SITE1 and EFEMP1 BINDING SITE2, designated SEQ ID: 1089 and SEQ ID: 1864 respectively, to the nucleotide sequence of VGAM308 RNA, herein designated VGAM RNA, also designated SEQ ID:643.

Another function of VGAM308 is therefore inhibition of EGF-containing Fibulin-like Extracellular Matrix Protein 1 (EFEMP1, Accession NM_{—}004105). Accordingly, utilities of VGAM308 include diagnosis, prevention and treatment of diseases and clinical conditions associated with EFEMP1. Hepatoma-derived Growth Factor (high-mobility group protein 1-like) (HDGF, Accession NM_{—}004494) is another VGAM308 host target gene. HDGF BINDING SITE is HOST TARGET binding site found in the 3′ untranslated region of mRNA encoded by HDGF, corresponding to a HOST TARGET binding site such as BINDING SITE I, BINDING SITE II or BINDING SITE III. Table 2 illustrates the complementarity of the nucleotide sequences of HDGF BINDING SITE, designated SEQ ID:1120, to the nucleotide sequence of VGAM308 RNA, herein designated VGAM RNA, also designated SEQ ID:643.

Another function of VGAM308 is therefore inhibition of Hepatoma-derived Growth Factor (high-mobility group protein 1-like) (HDGF, Accession NM_{—}004494), a gene which is a heparin-binding protein, with mitogenic activity for fibroblasts. Accordingly, utilities of VGAM308 include diagnosis, prevention and treatment of diseases and clinical conditions associated with HDGF. The function of HDGF has been established by previous studies. Nakamura et al. (1994) purified a novel hepatoma-derived growth factor from the conditioned medium of human hepatoma-derived cell line HuH-7. Molecular cloning of a cDNA from the cDNA library of the same cell line was done on the basis of the N-terminal amino acid sequence. The cDNA was 2.4 kb long and the deduced amino acid sequence contained 240 amino acids without a signal peptide-like N-terminal hydrophobic sequence. The primary sequence shared homology with the high mobility group-1 protein (OMIM Ref. No. 163905); they showed 23.4% amino acid identity and 35.6% amino acid similarity. Immunofluorescence study showed that HDGF is localized in the cytoplasm of hepatoma cells and northern blots showed that it is expressed ubiquitously in normal tissues and tumor cell lines. Nakamura et al. (1994) suggested that it is a novel heparin-binding protein with mitogenic activity for fibroblasts. HDGF is ubiquitously expressed in normal tissues and tumor cell lines. By PCR screening of a commercial monochromosomal hybrid panel, Wanschura et al. (1996) mapped HDGF to the X chromosome. By fluorescence in situ hybridization, they determined the sub-chromosomal localization to be Xq25. Whereas a major group of the HMG protein family has been mapped to chromosomal segments frequently involved in the tumorigenesis of benign solid tumors, no tumor association for the Xq25 region was known.

Full details of the abovementioned studies are described in the following publications, the disclosure of which are hereby incorporated by reference:
Nakamura, H.; Izumoto, Y.; Kambe, H.; Kuroda, T.; Mori, T.; Kawamura, K.; Yamamoto, H.; Kishimoto, T.: Molecular cloning of complementary DNA for a novel human hepatoma-derived growth factor: its homology with high mobility group-1 protein. J. Biol. Chem. 269: 25143-25149, 1994.; and
Wanschura, S.; Schoenmakers, E. F. P. M.; Huysmans, C.; Bartnitzke, S.; Van de Ven, W. J. M.; Bullerdiek, J.: Mapping of the gene encoding the human hepatoma-derived growth factor (HDG.

Further studies establishing the function and utilities of HDGF are found in John Hopkins OMIM database record ID 300043, and in sited publications numbered 1998-1999 listed in the bibliography section hereinbelow, which are also hereby incorporated by reference. Hepatocyte Growth Factor (hepapoietin A; scatter factor) (HGF, Accession XM_{—}168542) is another VGAM308 host target gene. HGF BINDING SITE is HOST TARGET binding site found in the 3′ untranslated region of mRNA encoded by HGF, corresponding to a HOST TARGET binding site such as BINDING SITE I, BINDING SITE II or BINDING SITE III. Table 2 illustrates the complementarity of the nucleotide sequences of HGF BINDING SITE, designated SEQ ID:3642, to the nucleotide sequence of VGAM308 RNA, herein designated VGAM RNA, also designated SEQ ID:643.

Another function of VGAM308 is therefore inhibition of Hepatocyte Growth Factor (hepapoietin A; scatter factor) (HGF, Accession XM_{—}168542), a gene which may be required for normal embryonic development; strongly similar to murine Hgf, has kringle domains. Accordingly, utilities of VGAM308 include diagnosis, prevention and treatment of diseases and clinical conditions associated with HGF. The function of HGF has been established by previous studies. Kilby et al. (1996) found that the protein and mRNA for both hepatocyte growth factor and its receptor (MET) are present in third trimester placentas, suggesting that HGF serves as a paracrine mediator to control placental development and growth. B cells develop in the bone marrow from progenitor cells that have been designated pre-pro-B cells, pro-B cells (no immunoglobulin, or Ig, chains chosen), pre-B cells (which have selected a heavy chain but not a light chain), and finally B cells (which express both heavy and light chains of the Ig molecule). Differentiation of pre-pro-B cells to pro-B cells requires signaling through IL7 receptor (IL7R; 146661) mediated by the pre-pro-B cell growth-stimulating factor (PPBSF), which consists of IL7 (OMIM Ref. No. 146660) and a 30-kD protein cofactor. By amino acid sequencing and RT-PCR analysis, Lai and Goldschneider (2001) determined that the PPBSF cofactor is the 30-kD beta chain of HGF (HGFB) produced independently of the 60-kD alpha chain of HGF. Formation of an IL7-HGFB heterodimer requires the presence of heparin sulfate. Functional analysis indicated that either IL7 or HGFB can maintain the viability of pre-pro-B cells, but only the heterodimer can stimulate their proliferation and differentiation into pro-B cells. Lai and Goldschneider (2001) concluded that PPBSF is a novel form of cytokine, a hybrid cytokine, consisting of the bioactive components of 2 unrelated cytokines. They proposed that through its heparin-binding and mitogenic properties, HGFB enables IL7 to participate in cognate interactions at the stromal cell surface and transduce signals effectively at low levels of IL7R. Animal model experiments lend further support to the function of HGF. Schmidt et al. (1995) and Uehara et al. (1995) produced targeted disruption of the HGF gene in mice and found that mice lacking the gene product fail to develop completely and die in utero. The mutation affects the embryonic liver, which is reduced in size and shows extensive loss of parenchymal cells. In addition, development of the placenta, particularly of trophoblast cells, is impaired. HGF/SF is thought to mediate a signal exchange between the mesenchyme and epithelia during mouse development. Both the HGF gene and the gene for its receptor, the product of the MET protooncogene, are expressed in many tissues during embryonic development and in the adult. The findings of these studies indicate that HGF/SF is an essential mediator of allantoic mesenchyme-trophoblastic epithelia interaction required for placental organogenesis.

It is appreciated that the abovementioned animal model for HGF is acknowledged by those skilled in the art as a scientifically valid animal model, as can be further appreciated from the publications sited hereinbelow.

Full details of the abovementioned studies are described in the following publications, the disclosure of which are hereby incorporated by reference:
Lai, L.; Goldschneider, I.: Cutting edge: identification of a hybrid cytokine consisting of IL-7 and the beta-chain of the hepatocyte growth factor/scatter factor. J. Immun. 167: 3550-3554, 2001.; and
Schmidt, C.; Bladt, F.; Goedecke, S.; Brinkmann, V.; Zschiesche, W.; Sharpe, M.; Gherardi, E.; Birchmeier, C.: Scatter factor/hepatocyte growth factor is essential for liver development.

Further studies establishing the function and utilities of HGF are found in John Hopkins OMIM database record ID 142409, and in sited publications numbered 2607-2610, 608, 2611-262 and 2923-2623 listed in the bibliography section hereinbelow, which are also hereby incorporated by reference. Nuclear Factor (erythroid-derived 2)-like 1 (NFE2Li, Accession NM_{—}003204) is another VGAM308 host target gene. NFE2L1 BINDING SITE is HOST TARGET binding site found in the 3′ untranslated region of mRNA encoded by NFE2Li, corresponding to a HOST TARGET binding site such as BINDING SITE I, BINDING SITE II or BINDING SITE III. Table 2 illustrates the complementarity of the nucleotide sequences of NFE2L1 BINDING SITE, designated SEQ ID:999, to the nucleotide sequence of VGAM308 RNA, herein designated VGAM RNA, also designated SEQ ID:643.

Another function of VGAM308 is therefore inhibition of Nuclear Factor (erythroid-derived 2)-like 1 (NFE2Li, Accession NM_{—}003204), a gene which may regulate expression of ferritin genes. Accordingly, utilities of VGAM308 include diagnosis, prevention and treatment of diseases and clinical conditions associated with NFE2L1. The function of NFE2L1 has been established by previous studies. Chan et al. (1993) devised a complementation assay in yeast to clone mammalian transcription activators and used it to identify a distinct human bZIP transcription factor, NFE2Li, which they designated NRF1 (NFE2-related factor-1) because of its similarities to NFE2 (OMIM Ref. No. 601490). Chan et al. (1995) showed that the NFE2L1 gene encodes a 742-amino acid protein with a different molecular weight than either the p45 subunit (NFE2) or the Maf protein subunit (MafF, MafG (OMIM Ref. No. 602020), or MafK (OMIM Ref. No. 600197)) of nuclear factor erythroid-2. Chan et al. (1993) found that NFE2L1 activates transcription via NFE2-binding sites in yeast cells. The ubiquitous expression pattern of NFE2L1 and the range of promoters containing the NFE2-binding motif suggested that this gene may play a role in the regulation of heme synthesis and ferritin genes Animal model experiments lend further support to the function of NFE2L1. To determine the function of Nrf1, Chan et al. (1998) disrupted the mouse gene by homologous recombination. Heterozygous Nfr1 mutant mice developed normally, were fertile, and showed no obvious abnormalities. Mice homozygous for the Nrf1 mutation suffered from anemia as a result of abnormal fetal liver erythropoiesis and died in utero at mid-late gestation. The authors did not detect defects in globin gene expression. Abnormal red cell production appeared to result from a defect in the fetal liver microenvironment specific for erythroid cells. Chan et al. (1998) suggested that target genes regulated by Nrf1 play an essential role during fetal liver hematopoiesis.

It is appreciated that the abovementioned animal model for NFE2L1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Chan, J. Y.; Kwong, M.; Lu, R.; Chang, J.; Wang, B.; Yen, T. S. B.; Kan, Y. W.: Targeted disruption of the ubiquitous CNC-bZIP transcription factor, Nrf-1, results in anemia and embryonic lethality in mice. EMBO J. 17: 1779-1787, 1998.; and
Chan, J. Y.; Han, X.-L.; Kan, Y. W.: Cloning of Nrf1, an NFE2-related transcription factor, by genetic selection in yeast. Proc. Nat. Acad. Sci. 90: 11371-11375, 1993.

Further studies establishing the function and utilities of NFE2L1 are found in John Hopkins OMIM database record ID 163260, and in sited publications numbered 2410-2416 listed in the bibliography section hereinbelow, which are also hereby incorporated by reference. Neuroligin 1 (NLGN1, Accession NM_{—}014932) is another VGAM308 host target gene. NLGN1 BINDING SITE is HOST TARGET binding site found in the 5′ untranslated region of mRNA encoded by NLGN1, corresponding to a HOST TARGET binding site such as BINDING SITE I, BINDING SITE II or BINDING SITE III. Table 2 illustrates the complementarity of the nucleotide sequences of NLGN1 BINDING SITE, designated SEQ ID:1592, to the nucleotide sequence of VGAM308 RNA, herein designated VGAM RNA, also designated SEQ ID:643.

Another function of VGAM308 is therefore inhibition of Neuroligin 1 (NLGN1, Accession NM_{—}014932), a gene which may trigger the de novo formation of presynaptic structure. Accordingly, utilities of VGAM308 include diagnosis, prevention and treatment of diseases and clinical conditions associated with NLGN1. The function of NLGN1 and its association with various diseases and clinical conditions, has been established by previous studies, as described hereinabove with reference to VGAM57. Solute Carrier Family 21 (prostaglandin transporter), Member 2 (SLC21A2, Accession NM_{—}005630) is another VGAM308 host target gene. SLC21A2 BINDING SITE is HOST TARGET binding site found in the 3′ untranslated region of mRNA encoded by SLC21A2, corresponding to a HOST TARGET binding site such as BINDING SITE I, BINDING SITE II or BINDING SITE III. Table 2 illustrates the complementarity of the nucleotide sequences of SLC21A2 BINDING SITE, designated SEQ ID:1232, to the nucleotide sequence of VGAM308 RNA, herein designated VGAM RNA, also designated SEQ ID:643.

Another function of VGAM308 is therefore inhibition of Solute Carrier Family 21 (prostaglandin transporter), Member 2 (SLC21A2, Accession NM_{—}005630), a gene which is a Prostaglandin transporter. Accordingly, utilities of VGAM308 include diagnosis, prevention and treatment of diseases and clinical conditions associated with SLC21A2. The function of SLC21A2 has been established by previous studies. At physiologic pH, prostaglandins (PGs) traverse biologic membranes poorly. Accordingly, PG transport is carrier-mediated in many tissues, including the lung, choroid plexus, liver, anterior chamber of the eye, vagina and uterus, and placenta. Kanai et al. (1995) cloned the rat prostaglandin transporter (symbolized PGT by them) and postulated 3 possible roles for the transporter. First, PGT might mediate the efflux of newly synthesized PGs from cells. Second, PGT might mediate epithelial PG transport. A third possible role of PGT is that of mediating PG clearance and degradation. Lu et al. (1996) favored the clearance role for PGT. Using a rat PGT probe on Northern blots of human kidney mRNA, they found evidence for the presence of a human PGT homolog. They screened a human kidney cDNA library and isolated human PGT. The gene encodes a 643-amino acid polypeptide with 82% identity to the rat protein. They expressed a full-length human cDNA clone in cultured cells and reported that both rat and human PGT transport PGD2, as well as PGE1, PGE2, and PGF2a. Although human PGT has cDNA and deduced amino acid sequences similar to those of the rat, the tissue distribution of mRNA transcripts is substantially broader in human. Additionally, the diversity of human PGT transcripts is greater and the affinity for thromboxane-2 is greater. Lu et al. (1996) found strong PGT mRNA expression in the human fetus. By PCR-based monochromosomal somatic cell hybrid mapping and fluorescence in situ hybridization, Lu and Schuster (1998) mapped the PGT gene to 3q21.

Full details of the abovementioned studies are described in the following publications, the disclosure of which are hereby incorporated by reference:
Kanai, N.; Lu, R.; Satriano, J. A.; Bao, Y.; Wolkoff, A. W.; Schuster, V. L.: Identification and characterization of a prostaglandin transporter. Science 268: 866-869, 1995.; and
Lu, R.; Kanai, N.; Bao, Y.; Schuster, V. L.: Cloning, in vitro expression, and tissue distribution of a human prostaglandin transporter cDNA (hPGT). J. Clin. Invest. 98: 1142-1149, 1996.

Further studies establishing the function and utilities of SLC21A2 are found in John Hopkins OMIM database record ID 601460, and in sited publications numbered 657-659 listed in the bibliography section hereinbelow, which are also hereby incorporated by reference. FHR5 (Accession NM_{—}030787) is another VGAM308 host target gene. FHR5 BINDING SITE is HOST TARGET binding site found in the 3′ untranslated region of mRNA encoded by FHR5, corresponding to a HOST TARGET binding site such as BINDING SITE I, BINDING SITE II or BINDING SITE III. Table 2 illustrates the complementarity of the nucleotide sequences of FHR5 BINDING SITE, designated SEQ ID:2166, to the nucleotide sequence of VGAM308 RNA, herein designated VGAM RNA, also designated SEQ ID:643.

Another function of VGAM308 is therefore inhibition of FHR5 (Accession NM_{—}030787). Accordingly, utilities of VGAM308 include diagnosis, prevention and treatment of diseases and clinical conditions associated with FHR5. FLJ13710 (Accession NM_{—}024817) is another VGAM308 host target gene. FLJ13710 BINDING SITE is HOST TARGET binding site found in the 3′ untranslated region of mRNA encoded by FLJ13710, corresponding to a HOST TARGET binding site such as BINDING SITE I, BINDING SITE II or BINDING SITE III. Table 2 illustrates the complementarity of the nucleotide sequences of FLJ13710 BINDING SITE, designated SEQ ID:2091, to the nucleotide sequence of VGAM308 RNA, herein designated VGAM RNA, also designated SEQ ID:643.

Another function of VGAM308 is therefore inhibition of FLJ13710 (Accession NM_{—}024817). Accordingly, utilities of VGAM308 include diagnosis, prevention and treatment of diseases and clinical conditions associated with FLJ13710. KIAA1155 (Accession XM_{—}030864) is another VGAM308 host target gene. KIAA1155 BINDING SITE is HOST TARGET binding site found in the 3′ untranslated region of mRNA encoded by KIAA1155, corresponding to a HOST TARGET binding site such as BINDING SITE I, BINDING SITE II or BINDING SITE III. Table 2 illustrates the complementarity of the nucleotide sequences of KIAA1155 BINDING SITE, designated SEQ ID:2628, to the nucleotide sequence of VGAM308 RNA, herein designated VGAM RNA, also designated SEQ ID:643.

Another function of VGAM308 is therefore inhibition of KIAA1155 (Accession XM_{—}030864). Accordingly, utilities of VGAM308 include diagnosis, prevention and treatment of diseases and clinical conditions associated with KIAA1155. SEF (Accession XM_{—}045300) is another VGAM308 host target gene. SEF BINDING SITE is HOST TARGET binding site found in the 3′ untranslated region of mRNA encoded by SEF, corresponding to a HOST TARGET binding site such as BINDING SITE I, BINDING SITE II or BINDING SITE III. Table 2 illustrates the complementarity of the nucleotide sequences of SEF BINDING SITE, designated SEQ ID:2860, to the nucleotide sequence of VGAM308 RNA, herein designated VGAM RNA, also designated SEQ ID:643.

Another function of VGAM308 is therefore inhibition of SEF (Accession XM_{—}045300). Accordingly, utilities of VGAM308 include diagnosis, prevention and treatment of diseases and clinical conditions associated with SEF. Three Prime Repair Exonuclease 1 (TREX1, Accession NM_{—}033627) is another VGAM308 host target gene. TREX1 BINDING SITE is HOST TARGET binding site found in the 5′ untranslated region of mRNA encoded by TREX1, corresponding to a HOST TARGET binding site such as BINDING SITE I, BINDING SITE II or BINDING SITE III. Table 2 illustrates the complementarity of the nucleotide sequences of TREX1 BINDING SITE, designated SEQ ID:2337, to the nucleotide sequence of VGAM308 RNA, herein designated VGAM RNA, also designated SEQ ID:643.

Another function of VGAM308 is therefore inhibition of Three Prime Repair Exonuclease 1 (TREX1, Accession NM_{—}033627). Accordingly, utilities of VGAM308 include diagnosis, prevention and treatment of diseases and clinical conditions associated with TREX1. Tripartite Motif-containing 6 (TRIM6, Accession NM_{—}058166) is another VGAM308 host target gene. TRIM6 BINDING SITE is HOST TARGET binding site found in the 3′ untranslated region of mRNA encoded by TRIM6, corresponding to a HOST TARGET binding site such as BINDING SITE I, BINDING SITE II or BINDING SITE III. Table 2 illustrates the complementarity of the nucleotide sequences of TRIM6 BINDING SITE, designated SEQ ID:2366, to the nucleotide sequence of VGAM308 RNA, herein designated VGAM RNA, also designated SEQ ID:643.

Another function of VGAM308 is therefore inhibition of Tripartite Motif-containing 6 (TRIM6, Accession NM_{—}058166). Accordingly, utilities of VGAM308 include diagnosis, prevention and treatment of diseases and clinical conditions associated with TRIM6. VEST1 (Accession NM_{—}052958) is another VGAM308 host target gene. VEST1 BINDING SITE is HOST TARGET binding site found in the 3′ untranslated region of mRNA encoded by VEST1, corresponding to a HOST TARGET binding site such as BINDING SITE I, BINDING SITE II or BINDING SITE III. Table 2 illustrates the complementarity of the nucleotide sequences of VEST1 BINDING SITE, designated SEQ ID:2349, to the nucleotide sequence of VGAM308 RNA, herein designated VGAM RNA, also designated SEQ ID:643.

Another function of VGAM308 is therefore inhibition of VEST1 (Accession NM_{—}052958). Accordingly, utilities of VGAM308 include diagnosis, prevention and treatment of diseases and clinical conditions associated with VEST1. LOC149076 (Accession XM_{—}086415) is another VGAM308 host target gene. LOC149076 BINDING SITE is HOST TARGET binding site found in the 5′ untranslated region of mRNA encoded by LOC149076, corresponding to a HOST TARGET binding site such as BINDING SITE I, BINDING SITE II or BINDING SITE III. Table 2 illustrates the complementarity of the nucleotide sequences of LOC149076 BINDING SITE, designated SEQ ID:3128, to the nucleotide sequence of VGAM308 RNA, herein designated VGAM RNA, also designated SEQ ID:643.

Another function of VGAM308 is therefore inhibition of LOC149076 (Accession XM_{—}086415). Accordingly, utilities of VGAM308 include diagnosis, prevention and treatment of diseases and clinical conditions associated with LOC149076. LOC254043 (Accession XM_{—}170800) is another VGAM308 host target gene. LOC254043 BINDING SITE is HOST TARGET binding site found in the 3′ untranslated region of mRNA encoded by LOC254043, corresponding to a HOST TARGET binding site such as BINDING SITE I, BINDING SITE II or BINDING SITE III. Table 2 illustrates the complementarity of the nucleotide sequences of LOC254043 BINDING SITE, designated SEQ ID:3667, to the nucleotide sequence of VGAM308 RNA, herein designated VGAM RNA, also designated SEQ ID:643.

Another function of VGAM308 is therefore inhibition of LOC254043 (Accession XM_{—}170800). Accordingly, utilities of VGAM308 include diagnosis, prevention and treatment of diseases and clinical conditions associated with LOC254043. FIG. 1 further provides a conceptual description of a novel bioinformatically detected viral gene of the present invention, referred to here as Viral Genomic Address Messenger 309 (VGAM309) viral gene, which modulates expression of respective host target genes thereof, the function and utility of which host target genes is known in the art.

VGAM309 is a novel bioinformatically detected regulatory, non protein coding, viral micro RNA (miRNA) gene. The method by which VGAM309 was detected is described hereinabove with reference to FIGS. 1-8.

VGAM309 gene, herein designated VGAM GENE, is a viral gene contained in the genome of Vaccinia Virus. VGAM309 host target gene, herein designated VGAM HOST TARGET GENE, is a human gene contained in the human genome.

VGAM309 gene encodes a VGAM309 precursor RNA, herein designated VGAM PRECURSOR RNA. Similar to other miRNA genes, and unlike most ordinary genes, VGAM309 precursor RNA does not encode a protein. A nucleotide sequence identical or highly similar to the nucleotide sequence of VGAM309 precursor RNA is designated SEQ ID:295, and is provided hereinbelow with reference to the sequence listing part. Nucleotide sequence SEQ ID:295 is located at position 177929 relative to the genome of Vaccinia Virus.

VGAM309 precursor RNA folds onto itself, forming VGAM309 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM309 folded precursor RNA into VGAM309 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 72%) nucleotide sequence of VGAM309 RNA is designated SEQ ID:644, and is provided hereinbelow with reference to the sequence listing part.

VGAM309 host target gene, herein designated VGAM HOST TARGET GENE, encodes a corresponding messenger RNA, VGAM309 host target RNA, herein designated VGAM HOST TARGET RNA. VGAM309 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM309 RNA, herein designated VGAM RNA, binds complementarily to one or more host target binding sites located in untranslated regions of VGAM309 host target RNA, herein designated VGAM HOST TARGET RNA. This complementary binding is due to the fact that the nucleotide sequence of VGAM309 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM309 RNA, herein designated VGAM RNA, may have a different number of host target binding sites in untranslated regions of a VGAM309 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM309 RNA, herein designated VGAM RNA, to host target binding sites on VGAM309 host target RNA, herein designated VGAM HOST TARGET RNA, such as BINDING SITE I, BINDING SITE II and BINDING SITE III, inhibits translation of VGAM309 host target RNA into VGAM309 host target protein, herein designated VGAM HOST TARGET PROTEIN. VGAM host target protein is therefore outlined by a broken line.

It is appreciated that VGAM309 host target gene, herein designated VGAM HOST TARGET GENE, in fact represents a plurality of VGAM309 host target genes. The mRNA of each one of this plurality of VGAM309 host target genes comprises one or more host target binding sites, each having a nucleotide sequence which is at least partly complementary to VGAM309 RNA, herein designated VGAM RNA, and which when bound by VGAM309 RNA causes inhibition of translation of respective one or more VGAM309 host target proteins.

It is further appreciated by one skilled in the art that the mode of translational inhibition illustrated by FIG. 1 with specific reference to translational inhibition exerted by VGAM309 gene, herein designated VGAM GENE, on one or more VGAM309 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM309 is inhibition of expression of host target genes, as part of a novel viral mechanism of attacking a host. Accordingly, utilities of VGAM309 include diagnosis, prevention and treatment of viral infection by Vaccinia Virus. Specific functions, and accordingly utilities, of VGAM309 correlate with, and may be deduced from, the identity of the host target genes which VGAM309 binds and inhibits, and the function of these host target genes, as elaborated hereinbelow.

Nucleotide sequences of the VGAM309 precursor RNA, herein designated VGAM PRECURSOR RNA, and of the ‘diced’ VGAM309 RNA, herein designated VGAM RNA, and a schematic representation of the secondary folding of VGAM309 folded precursor RNA, herein designated VGAM FOLDED PRECURSOR RNA, of VGAM309 are further described hereinbelow with reference to Table 1.

Nucleotide sequences of host target binding sites, such as BINDING SITE-I, BINDING SITE-II and BINDING SITE-III of FIG. 1, found on VGAM309 host target RNA, and schematic representation of the complementarity of each of these host target binding sites to VGAM309 RNA, herein designated VGAM RNA, are described hereinbelow with reference to Table 2.

As mentioned hereinabove with reference to FIG. 1, a function of VGAM309 gene, herein designated VGAM is inhibition of expression of VGAM309 target genes. It is appreciated that specific functions, and accordingly utilities, of VGAM309 correlate with, and may be deduced from, the identity of the target genes which VGAM309 binds and inhibits, and the function of these target genes, as elaborated hereinbelow.

BUB3 Budding Uninhibited By Benzimidazoles 3 Homolog (yeast) (BUB3, Accession NM_{—}004725) is a VGAM309 host target gene. BUB3 BINDING SITE is HOST TARGET binding site found in the 3′ untranslated region of mRNA encoded by BUB3, corresponding to a HOST TARGET binding site such as BINDING SITE I, BINDING SITE II or BINDING SITE III. Table 2 illustrates the complementarity of the nucleotide sequences of BUB3 BINDING SITE, designated SEQ ID:1149, to the nucleotide sequence of VGAM309 RNA, herein designated VGAM RNA, also designated SEQ ID:644.

A function of VGAM309 is therefore inhibition of BUB3 Budding Uninhibited By Benzimidazoles 3 Homolog (yeast) (BUB3, Accession NM_{—}004725), a gene which has a role in the mitotic spindle checkpoint. Accordingly, utilities of VGAM309 include diagnosis, prevention and treatment of diseases and clinical conditions associated with BUB3. The function of BUB3 has been established by previous studies. A feedback control mechanism, or cell cycle checkpoint, delays the onset of anaphase until all the chromosomes are correctly aligned on the mitotic spindle. Mutations in the S. cerevisiae BUB and MAD genes abolish this checkpoint, such that mutant cells fail to undergo mitotic arrest in response to spindle damage. The yeast BUB1 (see OMIM Ref. No. 602452) gene encodes a protein kinase that can bind and phosphorylate BUB3. Mammalian BUB1 localizes to the kinetochore of unaligned chromosomes. To further characterize the role of BUB1 in mitosis, Taylor et al. (1998) searched an EST database to identify the human homolog of BUB3. They identified a partial human BUB3 cDNA and used a PCR strategy to isolate a full-length cDNA. The predicted 328-amino acid human protein shares approximately 34% identity with yeast BUB3. Both proteins contain 4 WD repeats. When expressed in mammalian cells, a chimeric GFP-BUB3 protein localized to kinetochores before chromosome alignment. Using deletion analysis, the authors identified a domain of BUB1 that is required both for binding BUB3 and for kinetochore localization of BUB1. Taylor et al. (1998) reported that a similar domain in BUBR1 (OMIM Ref. No. 602860) mediates binding to BUB3. They suggested that the BUB and MAD proteins may be part of a large protein complex that is recruited to unattached kinetochores and that dissociates from kinetochores upon achieving correct bipolar attachment. Animal model experiments lend further support to the function of BUB3. By gene-targeting techniques, Kalitsis et al. (2000) disrupted the Bub3 gene in mice, which resulted in embryonic lethality prior to day 8.5 postcoitum (pc) in homozygous mutants. Mutant embryos appeared normal at day 3.5 pc but rapidly degenerated. An observed accumulation of mitotic errors suggested that Bub3 is essential for normal mitosis and for early embryonic development in the mouse.

It is appreciated that the abovementioned animal model for BUB3 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Kalitsis, P.; Earle, E.; Fowler, K. J.; Choo, K. H. A.: Bub3 gene disruption in mice reveals essential mitotic spindle checkpoint function during early embryogenesis. Genes Dev. 14: 2277-2282, 2000.; and
Taylor, S. S.; Ha, E.; McKeon, F.: The human homologue of Bub3 is required for kinetochore localization of Bub1 and a Mad3/Bub1-related protein kinase. J. Cell Biol. 142: 1-11, 1998.

Further studies establishing the function and utilities of BUB3 are found in John Hopkins OMIM database record ID 603719, and in sited publications numbered 63 and 1225 listed in the bibliography section hereinbelow, which are also hereby incorporated by reference. Chloride Channel 6 (CLCN6, Accession NM_{—}001286) is another VGAM309 host target gene. CLCN6 BINDING SITE1 through CLCN6 BINDING SITE3 are HOST TARGET binding sites found in untranslated regions of mRNA encoded by CLCN6, corresponding to HOST TARGET binding sites such as BINDING SITE I, BINDING SITE II or BINDING SITE III. Table 2 illustrates the complementarity of the nucleotide sequences of CLCN6 BINDING SITE1 through CLCN6 BINDING SITE3, designated SEQ ID:815, SEQ ID: 1957 and SEQ ID: 1958 respectively, to the nucleotide sequence of VGAM309 RNA, herein designated VGAM RNA, also designated SEQ ID:644.

Another function of VGAM309 is therefore inhibition of Chloride Channel 6 (CLCN6, Accession NM_{—}001286), a gene which is a voltage-gated chloride channel. Accordingly, utilities of VGAM309 include diagnosis, prevention and treatment of diseases and clinical conditions associated with CLCN6. The function of CLCN6 has been established by previous studies. Members of the mammalian CLCN family of voltage-gated chloride channels display differential tissue distribution and perform diverse functions. Nomura et al. (1994) identified a partial human CLCN6 cDNA, which they called KIAA0046. Northern blot analysis revealed that CLCN6 was expressed widely. Using the partial cDNA sequence of Nomura et al. (1994), Brandt and Jentsch (1995) cloned human cerebral cortex cDNAs that covered the entire CLCN6 coding region. The predicted 869-amino acid protein was called CLC6 by them. The amino acid sequence of CLCN6 is 45% identical to that of CLCN7 (OMIM Ref. No. 602727) but only 23 to 29% identical to the sequences of other known CLCNs. Therefore, Brandt and Jentsch (1995) stated that CLCN6 and CLCN7 together define a new branch of the chloride channel protein family. By Northern blot analysis, Brandt and Jentsch (1995) found that CLCN6 was expressed as an approximately 6-kb mRNA in all tissues examined. Eggermont et al. (1997) identified 4 different CLCN6 cDNAs that represent alternatively spliced transcripts. Nomura et al. (1994) mapped the CLCN6 gene to chromosome 1 using a somatic cell hybrid panel. By fluorescence in situ hybridization, Brandt and Jentsch (1995) refined the localization of the CLCN6 gene to 1p36. They noted that 2 genes encoding kidney-specific chloride channels, CLCNKA (OMIM Ref. No. 602024) and CLCNKB (OMIM Ref. No. 602023), also map to 1p36.

Full details of the abovementioned studies are described in the following publications, the disclosure of which are hereby incorporated by reference:
REFERENCES 1. Brandt, S.; Jentsch, T. J.: ClC-6 and ClC-7 are two novel broadly expressed members of the CLC chloride channel family. FEBS Lett. 377: 15-20, 1995.; and
Eggermont, J.; Buyse, G.; Voets, T.; Tytgat, J.; De Smedt, H.; Droogmans, G.: Alternative splicing of ClC-6 (a member of the ClC chloride-channel family) transcripts generates three tr.

Further studies establishing the function and utilities of CLCN6 are found in John Hopkins OMIM database record ID 602726, and in sited publications numbered 1939-194 and 511 listed in the bibliography section hereinbelow, which are also hereby incorporated by reference. CTP Synthase (CTPS, Accession XM_{—}114141) is another VGAM309 host target gene. CTPS BINDING SITE is HOST TARGET binding site found in the 3′ untranslated region of mRNA encoded by CTPS, corresponding to a HOST TARGET binding site such as BINDING SITE I, BINDING SITE II or BINDING SITE III. Table 2 illustrates the complementarity of the nucleotide sequences of CTPS BINDING SITE, designated SEQ ID:3441, to the nucleotide sequence of VGAM309 RNA, herein designated VGAM RNA, also designated SEQ ID:644.

Another function of VGAM309 is therefore inhibition of CTP Synthase (CTPS, Accession XM_{—}114141), a gene which is important in the biosynthesis of phospholipids and nucleic acids. Accordingly, utilities of VGAM309 include diagnosis, prevention and treatment of diseases and clinical conditions associated with CTPS. The function of CTPS has been established by previous studies. The catalytic conversion of UTP to CTP is accomplished by the enzyme cytidine-5-prime-triphosphate synthetase (UTP: L-glutamine amido ligase; EC 6.3.4.2). The enzyme is important in the biosynthesis of phospholipids and nucleic acids, and plays a key role in cell growth, development, and tumorigenesis. Thomas et al. (1989) isolated a cDNA clone of the CTP synthetase gene from a rat liver cDNA library. It is a key regulatory enzyme in pyrimidine biosynthesis. These authors have isolated both cDNA and genomic gene sequences from the rat and Chinese hamster. Yamauchi et al. (1990) cloned the CTPS gene and showed that the open reading frame encodes 591 amino acids that have a striking degree of similarity to the structural gene in E. coli Yamauchi et al. (1991) assigned the structural gene to 1p by study of a panel of human/rodent somatic cell hybrids and the CTPS cDNA. By a method of mapping that combines fluorescence in situ hybridization with replicated prometaphase R-bands (Takahashi et al., 1990), Takahashi et al. (1991) mapped the CTPS gene to 1p34.3-p34.1. By high-resolution banding analysis, they further narrowed the assignment to 1p34.1; see Yamauchi et al. (1991). The genomic sequence is distributed in 19 exons covering about 35 kb. Mutations eliminating the feedback regulation of CTPS result in multidrug resistance and mutator phenotype in Chinese hamster ovary (CHO) cells. The region to which the CTPS gene has been mapped is the location of breakpoints involved in several tumor types. Yamauchi et al. (1993) found that inactivating mutations clustered in a highly conserved region of the gene make it feasible to assess the role of such mutations in the development of drug resistance encountered in the treatment of malignant disease and not readily explained by altered expression of the multidrug resistance genes (e.g., 171050).

Full details of the abovementioned studies are described in the following publications, the disclosure of which are hereby incorporated by reference:
Yamauchi, M.; Yamauchi, N.; Phear, G.; Spurr, N. K.; Martinsson, T.; Weith, A.; Meuth, M.: Genomic organization and chromosomal localization of the human CTP synthetase gene (CTPS). Genomics 11: 1088-1096, 1991.; and
Whelan, J.; Phear, G.; Yamauchi, M.; Meuth, M.: Clustered base substitutions in CTP synthetase conferring drug resistance in Chinese hamster ovary cells. Nature Genet. 3: 317-322, 1993.

Further studies establishing the function and utilities of CTPS are found in John Hopkins OMIM database record ID 123860, and in sited publications numbered 3061-3066, and 897 listed in the bibliography section hereinbelow, which are also hereby incorporated by reference. Retinoblastoma 1 (including osteosarcoma) (RB1, Accession XM_{—}165641) is another VGAM309 host target gene. RB1 BINDING SITE is HOST TARGET binding site found in the 3′ untranslated region of mRNA encoded by RB1, corresponding to a HOST TARGET binding site such as BINDING SITE I, BINDING SITE II or BINDING SITE III. Table 2 illustrates the complementarity of the nucleotide sequences of RB1 BINDING SITE, designated SEQ ID:3505, to the nucleotide sequence of VGAM309 RNA, herein designated VGAM RNA, also designated SEQ ID:644.

Another function of VGAM309 is therefore inhibition of Retinoblastoma 1 (including osteosarcoma) (RB1, Accession XM_{—}165641), a gene which probably acts as a regulator of other genes. Accordingly, utilities of VGAM309 include diagnosis, prevention and treatment of diseases and clinical conditions associated with RB1. The function of RB1 has been established by previous studies. Retinoblastoma (RB) is an embryonic neoplasm of retinal origin. It almost always presents in early childhood and is often bilateral. Spontaneous regression (OMIM Ref. No. ‘cure’) occurs in some cases. Connolly et al. (1983) reported a 4-generation family with 3 patterns of expression of the retinoblastoma gene: frank retinoblastoma, unilateral or bilateral; retinoma; and no visible retinal pathology except for ‘normal degeneration’ with age. (‘Paving stone degeneration’ of the type observed in 2 of 3 RB carriers, aged 49 and 59, is said by Duane (1980) to occur in about 20% of the adult population.) Gallie and Phillips (1982) described benign lesions in the retina in retinoblastoma patients. The distinctive characteristics of these lesions, referred to by the authors as retinomas, included a translucent, grayish retinal mass protruding into the vitreous, ‘cottage-cheese’ calcification in 75%, and retinal pigment epithelial migration and proliferation in 60%. They suggested that retinomas represent not the heterozygous state postulated by the Knudson 2-stage model of carcinogenesis but rather the homozygous state occurring in differentiated cell(s). Gallie et al. (1982) suggested that retinomas represent either spontaneous regression of a retinoblastoma or a benign manifestation of the RB gene. Animal model experiments lend further support to the function of RB1. Windle et al. (1990) created transgenic mice by microinjecting fertilized ova with a chimeric gene containing the protein coding region of the SV40 T antigen (Tag) driven by the promoter of the luteinizing hormone beta-subunit gene. One of the male founders developed bilateral retinoblastomas at about age 5 months. The phenotype was heritable with complete penetrance in transgenic offspring in whom the tumors were first observed at about 2 months. Windle et al. (1990) demonstrated specific association between p105(Rb) and T antigen in mouse retinoblastoma tumor cells. Thus, evidence is provided for oncogenesis due to the ocular-specific expression of an Rb-binding oncoprotein that can functionally inactivate the Rb protein.

It is appreciated that the abovementioned animal model for RB1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Connolly, M. J.; Payne, R. H.; Johnson, G.; Gallie, B. L.; Allderdice, P. W.; Marshall, W. H.; Lawton, R. D.: Familial, EsD-linked, retinoblastoma with reduced penetrance and variable expressivity. Hum. Genet. 65: 122-124, 1983.; and
Gallie, B. L.; Ellsworth, R. M.; Abramson, D. M.; Phillips, R. A.: Retinoma: spontaneous regression of retinoblastoma or benign manifestation of the mutation? Brit. J. Cancer 45: 513-5.

Further studies establishing the function and utilities of RB1 are found in John Hopkins OMIM database record ID 180200, and in sited publications numbered 2549-2560, 2281-2282, 7-13, 545-565, 1125-571, 150-156, 1060-160, 737-175, 1297-1309, 3 and 1310-1317 listed in the bibliography section hereinbelow, which are also hereby incorporated by reference. Retinitis Pigmentosa 2 (X-linked recessive) (RP2, Accession NM_{—}006915) is another VGAM309 host target gene. RP2 BINDING SITE is HOST TARGET binding site found in the 3′ untranslated region of mRNA encoded by RP2, corresponding to a HOST TARGET binding site such as BINDING SITE I, BINDING SITE II or BINDING SITE III. Table 2 illustrates the complementarity of the nucleotide sequences of RP2 BINDING SITE, designated SEQ ID:1339, to the nucleotide sequence of VGAM309 RNA, herein designated VGAM RNA, also designated SEQ ID:644.

Another function of VGAM309 is therefore inhibition of Retinitis Pigmentosa 2 (X-linked recessive) (RP2, Accession NM_{—}006915). Accordingly, utilities of VGAM309 include diagnosis, prevention and treatment of diseases and clinical conditions associated with RP2. Wolf-Hirschhorn Syndrome Candidate 1 (WHSC1, Accession NM_{—}007331) is another VGAM309 host target gene. WHSC1 BINDING SITE is HOST TARGET binding site found in the 3′ untranslated region of mRNA encoded by WHSC1, corresponding to a HOST TARGET binding site such as BINDING SITE I, BINDING SITE II or BINDING SITE III. Table 2 illustrates the complementarity of the nucleotide sequences of WHSC1 BINDING SITE, designated SEQ ID:1394, to the nucleotide sequence of VGAM309 RNA, herein designated VGAM RNA, also designated SEQ ID:644.

Another function of VGAM309 is therefore inhibition of Wolf-Hirschhorn Syndrome Candidate 1 (WHSC1, Accession NM_{—}007331), a gene which binds covalently to and repairs g/t mismatches. Accordingly, utilities of VGAM309 include diagnosis, prevention and treatment of diseases and clinical conditions associated with WHSC1. The function of WHSC1 and its association with various diseases and clinical conditions, has been established by previous studies, as described hereinabove with reference to VGAM234. ARHGAP11A (Accession NM_{—}014783) is another VGAM309 host target gene. ARHGAP11A BINDING SITE is HOST TARGET binding site found in the 3′ untranslated region of mRNA encoded by ARHGAP11A, corresponding to a HOST TARGET binding site such as BINDING SITE I, BINDING SITE II or BINDING SITE III. Table 2 illustrates the complementarity of the nucleotide sequences of ARHGAP11A BINDING SITE, designated SEQ ID:1546, to the nucleotide sequence of VGAM309 RNA, herein designated VGAM RNA, also designated SEQ ID:644.

Another function of VGAM309 is therefore inhibition of ARHGAP11A (Accession NM_{—}014783). Accordingly, utilities of VGAM309 include diagnosis, prevention and treatment of diseases and clinical conditions associated with ARHGAP11A. FLJ20533 (Accession NM_{—}017866) is another VGAM309 host target gene. FLJ20533 BINDING SITE is HOST TARGET binding site found in the 3′ untranslated region of mRNA encoded by FLJ20533, corresponding to a HOST TARGET binding site such as BINDING SITE I, BINDING SITE II or BINDING SITE III. Table 2 illustrates the complementarity of the nucleotide sequences of FLJ20533 BINDING SITE, designated SEQ ID:1758, to the nucleotide sequence of VGAM309 RNA, herein designated VGAM RNA, also designated SEQ ID:644.

Another function of VGAM309 is therefore inhibition of FLJ20533 (Accession NM_{—}017866). Accordingly, utilities of VGAM309 include diagnosis, prevention and treatment of diseases and clinical conditions associated with FLJ20533. MGC16063 (Accession NM_{—}053047) is another VGAM309 host target gene. MGC16063 BINDING SITE is HOST TARGET binding site found in the 3′ untranslated region of mRNA encoded by MGC16063, corresponding to a HOST TARGET binding site such as BINDING SITE I, BINDING SITE II or BINDING SITE III. Table 2 illustrates the complementarity of the nucleotide sequences of MGC16063 BINDING SITE, designated SEQ ID:2356, to the nucleotide sequence of VGAM309 RNA, herein designated VGAM RNA, also designated SEQ ID:644.

Another function of VGAM309 is therefore inhibition of MGC16063 (Accession NM_{—}053047). Accordingly, utilities of VGAM309 include diagnosis, prevention and treatment of diseases and clinical conditions associated with MGC16063. Sema Domain, Immunoglobulin Domain (Ig), Short Basic Domain, Secreted, (semaphorin) 3C (SEMA3C, Accession NM_{—}006379) is another VGAM309 host target gene. SEMA3C BINDING SITE is HOST TARGET binding site found in the 3′ untranslated region of mRNA encoded by SEMA3C, corresponding to a HOST TARGET binding site such as BINDING SITE I, BINDING SITE II or BINDING SITE III. Table 2 illustrates the complementarity of the nucleotide sequences of SEMA3C BINDING SITE, designated SEQ ID:1293, to the nucleotide sequence of VGAM309 RNA, herein designated VGAM RNA, also designated SEQ ID:644.

Another function of VGAM309 is therefore inhibition of Sema Domain, Immunoglobulin Domain (Ig), Short Basic Domain, Secreted, (semaphorin) 3C (SEMA3C, Accession NM_{—}006379). Accordingly, utilities of VGAM309 include diagnosis, prevention and treatment of diseases and clinical conditions associated with SEMA3C. LOC135293 (Accession XM_{—}072402) is another VGAM309 host target gene. LOC135293 BINDING SITE is HOST TARGET binding site found in the 3′ untranslated region of mRNA encoded by LOC135293, corresponding to a HOST TARGET binding site such as BINDING SITE I, BINDING SITE II or BINDING SITE III. Table 2 illustrates the complementarity of the nucleotide sequences of LOC135293 BINDING SITE, designated SEQ ID:3047, to the nucleotide sequence of VGAM309 RNA, herein designated VGAM RNA, also designated SEQ ID:644.

Another function of VGAM309 is therefore inhibition of LOC135293 (Accession XM_{—}072402). Accordingly, utilities of VGAM309 include diagnosis, prevention and treatment of diseases and clinical conditions associated with LOC135293. LOC153883 (Accession XM_{—}087798) is another VGAM309 host target gene. LOC153883 BINDING SITE is HOST TARGET binding site found in the 3′ untranslated region of mRNA encoded by LOC153883, corresponding to a HOST TARGET binding site such as BINDING SITE I, BINDING SITE II or BINDING SITE III. Table 2 illustrates the complementarity of the nucleotide sequences of LOC153883 BINDING SITE, designated SEQ ID:3182, to the nucleotide sequence of VGAM309 RNA, herein designated VGAM RNA, also designated SEQ ID:644.

Another function of VGAM309 is therefore inhibition of LOC153883 (Accession XM_{—}087798). Accordingly, utilities of VGAM309 include diagnosis, prevention and treatment of diseases and clinical conditions associated with LOC153883. LOC199692 (Accession NM_{—}145295) is another VGAM309 host target gene. LOC199692 BINDING SITE is HOST TARGET binding site found in the 3′ untranslated region of mRNA encoded by LOC199692, corresponding to a HOST TARGET binding site such as BINDING SITE I, BINDING SITE II or BINDING SITE III. Table 2 illustrates the complementarity of the nucleotide sequences of LOC199692 BINDING SITE, designated SEQ ID:2521, to the nucleotide sequence of VGAM309 RNA, herein designated VGAM RNA, also designated SEQ ID:644.

Another function of VGAM309 is therefore inhibition of LOC199692 (Accession NM_{—}145295). Accordingly, utilities of VGAM309 include diagnosis, prevention and treatment of diseases and clinical conditions associated with LOC199692. FIG. 1 further provides a conceptual description of a novel bioinformatically detected viral gene of the present invention, referred to here as Viral Genomic Address Messenger 310 (VGAM310) viral gene, which modulates expression of respective host target genes thereof, the function and utility of which host target genes is known in the art.

VGAM310 is a novel bioinformatically detected regulatory, non protein coding, viral micro RNA (miRNA) gene. The method by which VGAM310 was detected is described hereinabove with reference to FIGS. 1-8.

VGAM310 gene, herein designated VGAM GENE, is a viral gene contained in the genome of Vaccinia Virus. VGAM310 host target gene, herein designated VGAM HOST TARGET GENE, is a human gene contained in the human genome.

VGAM310 gene encodes a VGAM310 precursor RNA, herein designated VGAM PRECURSOR RNA. Similar to other miRNA genes, and unlike most ordinary genes, VGAM310 precursor RNA does not encode a protein. A nucleotide sequence identical or highly similar to the nucleotide sequence of VGAM310 precursor RNA is designated SEQ ID:296, and is provided hereinbelow with reference to the sequence listing part. Nucleotide sequence SEQ ID:296 is located at position 9389 relative to the genome of Vaccinia Virus.

VGAM310 precursor RNA folds onto itself, forming VGAM310 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM310 folded precursor RNA into VGAM310 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 70%) nucleotide sequence of VGAM310 RNA is designated SEQ ID:645, and is provided hereinbelow with reference to the sequence listing part.

VGAM310 host target gene, herein designated VGAM HOST TARGET GENE, encodes a corresponding messenger RNA, VGAM310 host target RNA, herein designated VGAM HOST TARGET RNA. VGAM310 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM310 RNA, herein designated VGAM RNA, binds complementarily to one or more host target binding sites located in untranslated regions of VGAM310 host target RNA, herein designated VGAM HOST TARGET RNA. This complementary binding is due to the fact that the nucleotide sequence of VGAM310 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM310 RNA, herein designated VGAM RNA, may have a different number of host target binding sites in untranslated regions of a VGAM310 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM310 RNA, herein designated VGAM RNA, to host target binding sites on VGAM310 host target RNA, herein designated VGAM HOST TARGET RNA, such as BINDING SITE I, BINDING SITE II and BINDING SITE III, inhibits translation of VGAM310 host target RNA into VGAM310 host target protein, herein designated VGAM HOST TARGET PROTEIN. VGAM host target protein is therefore outlined by a broken line.

It is appreciated that VGAM310 host target gene, herein designated VGAM HOST TARGET GENE, in fact represents a plurality of VGAM310 host target genes. The mRNA of each one of this plurality of VGAM310 host target genes comprises one or more host target binding sites, each having a nucleotide sequence which is at least partly complementary to VGAM310 RNA, herein designated VGAM RNA, and which when bound by VGAM310 RNA causes inhibition of translation of respective one or more VGAM310 host target proteins.

It is further appreciated by one skilled in the art that the mode of translational inhibition illustrated by FIG. 1 with specific reference to translational inhibition exerted by VGAM310 gene, herein designated VGAM GENE, on one or more VGAM310 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM310 is inhibition of expression of host target genes, as part of a novel viral mechanism of attacking a host. Accordingly, utilities of VGAM310 include diagnosis, prevention and treatment of viral infection by Vaccinia Virus. Specific functions, and accordingly utilities, of VGAM310 correlate with, and may be deduced from, the identity of the host target genes which VGAM310 binds and inhibits, and the function of these host target genes, as elaborated hereinbelow.

Nucleotide sequences of the VGAM310 precursor RNA, herein designated VGAM PRECURSOR RNA, and of the ‘diced’ VGAM310 RNA, herein designated VGAM RNA, and a schematic representation of the secondary folding of VGAM310 folded precursor RNA, herein designated VGAM FOLDED PRECURSOR RNA, of VGAM310 are further described hereinbelow with reference to Table 1.

Nucleotide sequences of host target binding sites, such as BINDING SITE-I, BINDING SITE-II and BINDING SITE-III of FIG. 1, found on VGAM310 host target RNA, and schematic representation of the complementarity of each of these host target binding sites to VGAM310 RNA, herein designated VGAM RNA, are described hereinbelow with reference to Table 2.

As mentioned hereinabove with reference to FIG. 1, a function of VGAM310 gene, herein designated VGAM is inhibition of expression of VGAM310 target genes. It is appreciated that specific functions, and accordingly utilities, of VGAM310 correlate with, and may be deduced from, the identity of the target genes which VGAM310 binds and inhibits, and the function of these target genes, as elaborated hereinbelow.

MGC5242 (Accession NM_{—}024033) is a VGAM310 host target gene. MGC5242 BINDING SITE is HOST TARGET binding site found in the 5′ untranslated region of mRNA encoded by MGC5242, corresponding to a HOST TARGET binding site such as BINDING SITE I, BINDING SITE II or BINDING SITE III. Table 2 illustrates the complementarity of the nucleotide sequences of MGC5242 BINDING SITE, designated SEQ ID:2042, to the nucleotide sequence of VGAM310 RNA, herein designated VGAM RNA, also designated SEQ ID:645.

A function of VGAM310 is therefore inhibition of MGC5242 (Accession NM_{—}024033). Accordingly, utilities of VGAM310 include diagnosis, prevention and treatment of diseases and clinical conditions associated with MGC5242. LOC197259 (Accession XM_{—}113849) is another VGAM310 host target gene. LOC197259 BINDING SITE is HOST TARGET binding site found in the 3′ untranslated region of mRNA encoded by LOC197259, corresponding to a HOST TARGET binding site such as BINDING SITE I, BINDING SITE II or BINDING SITE III. Table 2 illustrates the complementarity of the nucleotide sequences of LOC197259 BINDING SITE, designated SEQ ID:3422, to the nucleotide sequence of VGAM310 RNA, herein designated VGAM RNA, also designated SEQ ID:645.

Another function of VGAM310 is therefore inhibition of LOC197259 (Accession XM_{—}113849). Accordingly, utilities of VGAM310 include diagnosis, prevention and treatment of diseases and clinical conditions associated with LOC197259. FIG. 1 further provides a conceptual description of a novel bioinformatically detected viral gene of the present invention, referred to here as Viral Genomic Address Messenger 311 (VGAM311) viral gene, which modulates expression of respective host target genes thereof, the function and utility of which host target genes is known in the art.

VGAM311 is a novel bioinformatically detected regulatory, non protein coding, viral micro RNA (miRNA) gene. The method by which VGAM311 was detected is described hereinabove with reference to FIGS. 1-8.

VGAM311 gene, herein designated VGAM GENE, is a viral gene contained in the genome of Vaccinia Virus. VGAM311 host target gene, herein designated VGAM HOST TARGET GENE, is a human gene contained in the human genome.

VGAM311 gene encodes a VGAM311 precursor RNA, herein designated VGAM PRECURSOR RNA. Similar to other miRNA genes, and unlike most ordinary genes, VGAM311 precursor RNA does not encode a protein. A nucleotide sequence identical or highly similar to the nucleotide sequence of VGAM311 precursor RNA is designated SEQ ID:297, and is provided hereinbelow with reference to the sequence listing part. Nucleotide sequence SEQ ID:297 is located at position 176858 relative to the genome of Vaccinia Virus.

VGAM311 precursor RNA folds onto itself, forming VGAM311 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM311 folded precursor RNA into VGAM311 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM311 RNA is designated SEQ ID:646, and is provided hereinbelow with reference to the sequence listing part.

VGAM311 host target gene, herein designated VGAM HOST TARGET GENE, encodes a corresponding messenger RNA, VGAM311 host target RNA, herein designated VGAM HOST TARGET RNA. VGAM 311 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM311 RNA, herein designated VGAM RNA, binds complementarily to one or more host target binding sites located in untranslated regions of VGAM311 host target RNA, herein designated VGAM HOST TARGET RNA. This complementary binding is due to the fact that the nucleotide sequence of VGAM311 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM311 RNA, herein designated VGAM RNA, may have a different number of host target binding sites in untranslated regions of a VGAM311 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM311 RNA, herein designated VGAM RNA, to host target binding sites on VGAM311 host target RNA, herein designated VGAM HOST TARGET RNA, such as BINDING SITE I, BINDING SITE II and BINDING SITE III, inhibits translation of VGAM311 host target RNA into VGAM311 host target protein, herein designated VGAM HOST TARGET PROTEIN. VGAM host target protein is therefore outlined by a broken line.

It is appreciated that VGAM311 host target gene, herein designated VGAM HOST TARGET GENE, in fact represents a plurality of VGAM311 host target genes. The mRNA of each one of this plurality of VGAM311 host target genes comprises one or more host target binding sites, each having a nucleotide sequence which is at least partly complementary to VGAM311 RNA, herein designated VGAM RNA, and which when bound by VGAM311 RNA causes inhibition of translation of respective one or more VGAM311 host target proteins.

It is further appreciated by one skilled in the art that the mode of translational inhibition illustrated by FIG. 1 with specific reference to translational inhibition exerted by VGAM311 gene, herein designated VGAM GENE, on one or more VGAM311 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM311 is inhibition of expression of host target genes, as part of a novel viral mechanism of attacking a host. Accordingly, utilities of VGAM311 include diagnosis, prevention and treatment of viral infection by Vaccinia Virus. Specific functions, and accordingly utilities, of VGAM311 correlate with, and may be deduced from, the identity of the host target genes which VGAM311 binds and inhibits, and the function of these host target genes, as elaborated hereinbelow.

Nucleotide sequences of the VGAM311 precursor RNA, herein designated VGAM PRECURSOR RNA, and of the ‘diced’ VGAM311 RNA, herein designated VGAM RNA, and a schematic representation of the secondary folding of VGAM311 folded precursor RNA, herein designated VGAM FOLDED PRECURSOR RNA, of VGAM311 are further described hereinbelow with reference to Table 1.

Nucleotide sequences of host target binding sites, such as BINDING SITE-I, BINDING SITE-II and BINDING SITE-III of FIG. 1, found on VGAM311 host target RNA, and schematic representation of the complementarity of each of these host target binding sites to VGAM311 RNA, herein designated VGAM RNA, are described hereinbelow with reference to Table 2.

As mentioned hereinabove with reference to FIG. 1, a function of VGAM311 gene, herein designated VGAM is inhibition of expression of VGAM311 target genes. It is appreciated that specific functions, and accordingly utilities, of VGAM311 correlate with, and may be deduced from, the identity of the target genes which VGAM311 binds and inhibits, and the function of these target genes, as elaborated hereinbelow.

Crystallin, Beta A4 (CRYBA4, Accession NM_{—}001886) is a VGAM311 host target gene. CRYBA4 BINDING SITE is HOST TARGET binding site found in the 3′ untranslated region of mRNA encoded by CRYBA4, corresponding to a HOST TARGET binding site such as BINDING SITE I, BINDING SITE II or BINDING SITE III. Table 2 illustrates the complementarity of the nucleotide sequences of CRYBA4 BINDING SITE, designated SEQ ID:865, to the nucleotide sequence of VGAM311 RNA, herein designated VGAM RNA, also designated SEQ ID:646.

A function of VGAM311 is therefore inhibition of Crystallin, Beta A4 (CRYBA4, Accession NM_{—}001886), a gene which is the dominant structural components of the vertebrate eye lens. Accordingly, utilities of VGAM311 include diagnosis, prevention and treatment of diseases and clinical conditions associated with CRYBA4. The function of CRYBA4 has been established by previous studies. In the ocular lens, 80 to 90% of the soluble protein fraction comprises crystallins. These proteins are thought to play an important role in the maintenance of the transparency and refractive index of the lens. In the mammalian lens, crystallins can be divided into alpha, beta, gamma, and delta families. These in turn can be subdivided into acidic and basic groups. The beta-A4 gene, an acidic crystallin, was mapped to 22q11.2-q13.1 by van Rens et al. (1991, 1992) by study of somatic cell hybrids including some containing translocated chromosome 22 segments. The CRYBB2 (OMIM Ref. No. 123620) and the CRYBB3 (OMIM Ref. No. 123630) genes have also been assigned to this region of chromosome 22. Hulsebos et al. (1995) demonstrated by interspecific backcross analysis that the homologs of the CRYBB2, CRYBB3, and CRYBA4 genes are located in the central region of mouse chromosome 5. Lampi et al. (1997) cloned the CRYBA4 gene, which they designated beta-A4, by 3-prime and 5-prime RACE-PCR of lens RNA with primers based on the sequence of bovine CRYBA4. The beta-A4 gene encodes a predicted 196-amino acid protein; the initial methionine is posttranslationally removed. The conserved crystallin domain is 92 to 94% identical to rat and bovine beta-A4, and there is an unrelated 10-amino acid N-terminal region (‘N-terminal extension’). The calculated 22,285-dalton molecular weight of the protein matched the value determined experimentally by mass spectrometry and by analysis of tryptic peptides. Lampi et al. (1997) found that there are 11 major soluble proteins in the young human lens, and that beta-A4 constitutes approximately 5% of the total

Full details of the abovementioned studies are described in the following publications, the disclosure of which are hereby incorporated by reference:
Hulsebos, T. J. M.; Jenkins, N. A.; Gilbert, D. J.; Copeland, N. G.: The beta crystallin genes on human chromosome 22 define a new region of homology with mouse chromosome 5. Genomics 25: 574-576, 1995.; and
Hulsebos, T. J. M.; Jenkins, N. A.; Gilbert, D. J.; Copeland, N. G.: The beta crystallin genes on human chromosome 22 define a new region of homology with mouse chromosome 5. Genomics 2.

Further studies establishing the function and utilities of CRYBA4 are found in John Hopkins OMIM database record ID 123631, and in sited publications numbered 2805-2808 listed in the bibliography section hereinbelow, which are also hereby incorporated by reference. Ubiquitin Carboxyl-terminal Esterase L1 (ubiquitin thiolesterase) (UCHL1, Accession NM_{—}004181) is another VGAM311 host target gene. UCHL1 BINDING SITE is HOST TARGET binding site found in the 3′ untranslated region of mRNA encoded by UCHL1, corresponding to a HOST TARGET binding site such as BINDING SITE I, BINDING SITE II or BINDING SITE III. Table 2 illustrates the complementarity of the nucleotide sequences of UCHL1 BINDING SITE, designated SEQ ID: 1093, to the nucleotide sequence of VGAM311 RNA, herein designated VGAM RNA, also designated SEQ ID:646.

Another function of VGAM311 is therefore inhibition of Ubiquitin Carboxyl-terminal Esterase L1 (ubiquitin thiolesterase) (UCHL1, Accession NM_{—}004181), a gene which is involved both in the processing of ubiquitin precursors and of ubiquinated proteins. Accordingly, utilities of VGAM311 include diagnosis, prevention and treatment of diseases and clinical conditions associated with UCHL1. The function of UCHL1 has been established by previous studies. Ubiquitin C-terminal hydrolase L1 represents 1 to 2% of total soluble brain protein (Wilkinson et al., 1989). Its occurrence in Lewy bodies and its function in the proteasome pathway make it a compelling candidate gene in Parkinson disease (OMIM Ref. No. 168600). In a German family with Parkinson disease, Leroy et al. (1998) identified a missense mutation in the UCHL1 gene, ile93 to met (193M; 191342.0001), which caused a partial loss of the catalytic activity of this thiol protease. They suggested that this could lead to aberrations in the proteolytic pathway and aggregation of proteins. Animal model experiments lend further support to the function of UCHL1. Kurihara et al. (2000) showed that mice homozygous for a targeted deletion of the related Uchl3 gene (OMIM Ref. No. 603090) are indistinguishable from wildtype. To assess whether the 2 hydrolases have redundant function, Kurihara et al. (2001) generated mice homozygous for both Uchl1(gad) and Uchl3(delta3-7). The double homozygotes weighed 30% less than single homozygotes and displayed an earlier onset of lethality, possibly due to dysphagia. Axonal degeneration of the nucleus tractus solitarius and area postrema of the medulla was noted in these mice. The double homozygotes also displayed a more severe axonal degeneration of the gracile tract of the medulla and spinal cord than had been observed in Uchl1(gad) single homozygotes. In addition, degeneration of dorsal root ganglia cell bodies was detected in both the double homozygotes and Uchl3(delta3-7) single homozygotes. Given that both Uchl1(gad) and Uchl3(delta3-7) single homozygotes displayed distinct degenerative defects that were exacerbated in the double homozygotes, the authors concluded that Uchl1 and Uchl3 may have both separate and overlapping functions in the maintenance of neurons of the gracile tract, nucleus tractus solitarius, and area postrema.

It is appreciated that the abovementioned animal model for UCHL1 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Kurihara, L. J.; Kikuchi, T.; Wada, K.; Tilghman, S. M.: Loss of Uch-L1 and Uch-L3 leads to neurodegeneration, posterior paralysis and dysphagia. Hum. Molec. Genet. 10: 1963-1970, 2001.; and
Leroy, E.; Boyer, R.; Auburger, G.; Leube, B.; Ulm, G.; Mezey, E.; Harta, G.; Brownstein, M. J.; Jonnalagada, S.; Chernova, T.; Dehejia, A.; Lavedan, C.; Gasser, T.; Steinbach, P. J.; W.

Further studies establishing the function and utilities of UCHL1 are found in John Hopkins OMIM database record ID 191342, and in sited publications numbered 2958-2970 listed in the bibliography section hereinbelow, which are also hereby incorporated by reference. BDG-29 (Accession XM_{—}051343) is another VGAM311 host target gene. BDG-29 BINDING SITE is HOST TARGET binding site found in the 3′ untranslated region of mRNA encoded by BDG-29, corresponding to a HOST TARGET binding site such as BINDING SITE I, BINDING SITE II or BINDING SITE III. Table 2 illustrates the complementarity of the nucleotide sequences of BDG-29 BINDING SITE, designated SEQ ID:2944, to the nucleotide sequence of VGAM311 RNA, herein designated VGAM RNA, also designated SEQ ID:646.

Another function of VGAM311 is therefore inhibition of BDG-29 (Accession XM_{—}051343). Accordingly, utilities of VGAM311 include diagnosis, prevention and treatment of diseases and clinical conditions associated with BDG-29. ERG-1 (Accession NM_{—}022034) is another VGAM311 host target gene. ERG-1 BINDING SITE is HOST TARGET binding site found in the 5′ untranslated region of mRNA encoded by ERG-1, corresponding to a HOST TARGET binding site such as BINDING SITE I, BINDING SITE II or BINDING SITE III. Table 2 illustrates the complementarity of the nucleotide sequences of ERG-1 BINDING SITE, designated SEQ ID: 1972, to the nucleotide sequence of VGAM311 RNA, herein designated VGAM RNA, also designated SEQ ID:646.

Another function of VGAM311 is therefore inhibition of ERG-1 (Accession NM_{—}022034). Accordingly, utilities of VGAM311 include diagnosis, prevention and treatment of diseases and clinical conditions associated with ERG-1. LOC199837 (Accession XM_{—}114034) is another VGAM311 host target gene. LOC199837 BINDING SITE is HOST TARGET binding site found in the 5′ untranslated region of mRNA encoded by LOC199837, corresponding to a HOST TARGET binding site such as BINDING SITE I, BINDING SITE II or BINDING SITE III. Table 2 illustrates the complementarity of the nucleotide sequences of LOC199837 BINDING SITE, designated SEQ ID:3434, to the nucleotide sequence of VGAM311 RNA, herein designated VGAM RNA, also designated SEQ ID:646.

Another function of VGAM311 is therefore inhibition of LOC199837 (Accession XM_{—}114034). Accordingly, utilities of VGAM311 include diagnosis, prevention and treatment of diseases and clinical conditions associated with LOC199837. FIG. 1 further provides a conceptual description of a novel bioinformatically detected viral gene of the present invention, referred to here as Viral Genomic Address Messenger 312 (VGAM312) viral gene, which modulates expression of respective host target genes thereof, the function and utility of which host target genes is known in the art.

VGAM312 is a novel bioinformatically detected regulatory, non protein coding, viral micro RNA (miRNA) gene. The method by which VGAM312 was detected is described hereinabove with reference to FIGS. 1-8.

VGAM312 gene, herein designated VGAM GENE, is a viral gene contained in the genome of Vaccinia Virus. VGAM312 host target gene, herein designated VGAM HOST TARGET GENE, is a human gene contained in the human genome.

VGAM312 gene encodes a VGAM312 precursor RNA, herein designated VGAM PRECURSOR RNA. Similar to other miRNA genes, and unlike most ordinary genes, VGAM312 precursor RNA does not encode a protein. A nucleotide sequence identical or highly similar to the nucleotide sequence of VGAM312 precursor RNA is designated SEQ ID:298, and is provided hereinbelow with reference to the sequence listing part. Nucleotide sequence SEQ ID:298 is located at position 177797 relative to the genome of Vaccinia Virus.

VGAM312 precursor RNA folds onto itself, forming VGAM312 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM312 folded precursor RNA into VGAM312 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM312 RNA is designated SEQ ID:647, and is provided hereinbelow with reference to the sequence listing part.

VGAM312 host target gene, herein designated VGAM HOST TARGET GENE, encodes a corresponding messenger RNA, VGAM312 host target RNA, herein designated VGAM HOST TARGET RNA. VGAM312 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM312 RNA, herein designated VGAM RNA, binds complementarily to one or more host target binding sites located in untranslated regions of VGAM312 host target RNA, herein designated VGAM HOST TARGET RNA. This complementary binding is due to the fact that the nucleotide sequence of VGAM312 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM312 RNA, herein designated VGAM RNA, may have a different number of host target binding sites in untranslated regions of a VGAM312 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM312 RNA, herein designated VGAM RNA, to host target binding sites on VGAM312 host target RNA, herein designated VGAM HOST TARGET RNA, such as BINDING SITE I, BINDING SITE II and BINDING SITE III, inhibits translation of VGAM312 host target RNA into VGAM312 host target protein, herein designated VGAM HOST TARGET PROTEIN. VGAM host target protein is therefore outlined by a broken line.

It is appreciated that VGAM312 host target gene, herein designated VGAM HOST TARGET GENE, in fact represents a plurality of VGAM312 host target genes. The mRNA of each one of this plurality of VGAM312 host target genes comprises one or more host target binding sites, each having a nucleotide sequence which is at least partly complementary to VGAM312 RNA, herein designated VGAM RNA, and which when bound by VGAM312 RNA causes inhibition of translation of respective one or more VGAM312 host target proteins.

It is further appreciated by one skilled in the art that the mode of translational inhibition illustrated by FIG. 1 with specific reference to translational inhibition exerted by VGAM312 gene, herein designated VGAM GENE, on one or more VGAM312 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM312 is inhibition of expression of host target genes, as part of a novel viral mechanism of attacking a host. Accordingly, utilities of VGAM312 include diagnosis, prevention and treatment of viral infection by Vaccinia Virus. Specific functions, and accordingly utilities, of VGAM312 correlate with, and may be deduced from, the identity of the host target genes which VGAM312 binds and inhibits, and the function of these host target genes, as elaborated hereinbelow.

Nucleotide sequences of the VGAM312 precursor RNA, herein designated VGAM PRECURSOR RNA, and of the ‘diced’ VGAM312 RNA, herein designated VGAM RNA, and a schematic representation of the secondary folding of VGAM312 folded precursor RNA, herein designated VGAM FOLDED PRECURSOR RNA, of VGAM312 are further described hereinbelow with reference to Table 1.

Nucleotide sequences of host target binding sites, such as BINDING SITE-I, BINDING SITE-II and BINDING SITE-III of FIG. 1, found on VGAM312 host target RNA, and schematic representation of the complementarity of each of these host target binding sites to VGAM312 RNA, herein designated VGAM RNA, are described hereinbelow with reference to Table 2.

As mentioned hereinabove with reference to FIG. 1, a function of VGAM312 gene, herein designated VGAM is inhibition of expression of VGAM312 target genes. It is appreciated that specific functions, and accordingly utilities, of VGAM312 correlate with, and may be deduced from, the identity of the target genes which VGAM312 binds and inhibits, and the function of these target genes, as elaborated hereinbelow.

Basic Helix-loop-helix Domain Containing, Class B, 3 (BHLHB3, Accession NM_{—}030762) is a VGAM312 host target gene. BHLHB3 BINDING SITE is HOST TARGET binding site found in the 3′ untranslated region of mRNA encoded by BHLHB3, corresponding to a HOST TARGET binding site such as BINDING SITE I, BINDING SITE II or BINDING SITE III. Table 2 illustrates the complementarity of the nucleotide sequences of BHLHB3 BINDING SITE, designated SEQ ID:2160, to the nucleotide sequence of VGAM312 RNA, herein designated VGAM RNA, also designated SEQ ID:647.

A function of VGAM312 is therefore inhibition of Basic Helix-loop-helix Domain Containing, Class B, 3 (BHLHB3, Accession NM_{—}030762), a gene which represses both basal and activated transcription. Accordingly, utilities of VGAM312 include diagnosis, prevention and treatment of diseases and clinical conditions associated with BHLHB3. The function of BHLHB3 and its association with various diseases and clinical conditions, has been established by previous studies, as described hereinabove with reference to VGAM127. Ceroid-lipofuscinosis, Neuronal 2, Late Infantile (Jansky-Bielschowsky disease) (CLN2, Accession NM_{—}000391) is another VGAM312 host target gene. CLN2 BINDING SITE is HOST TARGET binding site found in the 3′ untranslated region of mRNA encoded by CLN2, corresponding to a HOST TARGET binding site such as BINDING SITE I, BINDING SITE II or BINDING SITE III. Table 2 illustrates the complementarity of the nucleotide sequences of CLN2 BINDING SITE, designated SEQ ID:735, to the nucleotide sequence of VGAM312 RNA, herein designated VGAM RNA, also designated SEQ ID:647.

Another function of VGAM312 is therefore inhibition of Ceroid-lipofuscinosis, Neuronal 2, Late Infantile (Jansky-Bielschowsky disease) (CLN2, Accession NM_{—}000391). Accordingly, utilities of VGAM312 include diagnosis, prevention and treatment of diseases and clinical conditions associated with CLN2. Zinc Finger Protein 36, C3H Type-like 1 (ZFP36Li, Accession NM_{—}004926) is another VGAM312 host target gene. ZFP36L1 BINDING SITE is HOST TARGET binding site found in the 3′ untranslated region of mRNA encoded by ZFP36Li, corresponding to a HOST TARGET binding site such as BINDING SITE I, BINDING SITE II or BINDING SITE III. Table 2 illustrates the complementarity of the nucleotide sequences of ZFP36L1 BINDING SITE, designated SEQ ID:1165, to the nucleotide sequence of VGAM312 RNA, herein designated VGAM RNA, also designated SEQ ID:647.

Another function of VGAM312 is therefore inhibition of Zinc Finger Protein 36, C3H Type-like 1 (ZFP36Li, Accession NM_{—}004926), a gene which is a regulatory protein involved in regulating the response to growth factors. Accordingly, utilities of VGAM312 include diagnosis, prevention and treatment of diseases and clinical conditions associated with ZFP36L1. The function of ZFP36L1 has been established by previous studies. Bustin et al. (1994) cloned and characterized the ZFP36L1 gene, which they called ERF1, which is a member of the Tis11 family of early-response genes (see OMIM Ref. No. ZFP36; 190700). Members of this gene family contain a distinguishing putative zinc finger domain with a repeating cys-his motif and are induced by various agonists such as the phorbol ester TPA and the polypeptide mitogen EGF (OMIM Ref. No. 131530). The human gene was cloned using a rat homolog as a probe. The rat and human genes have conserved 5-prime and 3-prime UTRs and their promoters contain motifs seen in other early-response genes. The predicted rat and human proteins are 99% identical. Bustin et al. (1994) determined that the ZFP36L1 gene contains 2 exons and spans about 6 kb of genomic DNA including the promoter and UTRs. Ning et al. (1996) also cloned ZFP36Li, which they termed BERG36 (B-cell early response gene encoding a 36-kD protein). The deduced 338-amino acid BERG36 protein could be induced by treatment with calcium ionophore, and the induction could be blocked by treatment with interleukin-4 (IL4; 147780) but not by CD40 (TNFRSF5; 109535) ligation. Treatment of the Epstein-Barr virus-negative human Burkitt lymphoma cell line Ramos, which phenotypically resembles germinal center B cells, with BERG36-antisense or with IL4 or CD40 ligation protected the cells from ionophore-induced apoptosis. CD40 ligation also protected Ramos cells from apoptosis induced by inhibitors of macromolecular synthesis. Ning et al. (1996) concluded that BERG36 is a target of IL4 signaling for B-cell survival.

Full details of the abovementioned studies are described in the following publications, the disclosure of which are hereby incorporated by reference:
Bustin, S. A.; Xiao-Feng, N.; Barnard, R. C.; Kumar, V.; Pascall, J. C.; Brown, K. D.; Leigh, I. M.; Williams, N. S.; McKay, I. A.: Cloning and characterisation of ERF1, a human member of the Tis11 family of early-response genes. DNA Cell Biol. 13: 449-459, 1994.; and
Ning, Z.-Q.; Norton, J. D.; Li, J.; Murphy, J. J.: Distinct mechanisms for rescue from apoptosis in Ramos human B cells by signaling through CD40 and interleukin-4 receptor: a role for.

Further studies establishing the function and utilities of ZFP36L1 are found in John Hopkins OMIM database record ID 601064, and in sited publications numbered 1783-1786 listed in the bibliography section hereinbelow, which are also hereby incorporated by reference. Chromosome 8 Open Reading Frame 2 (C8orf2, Accession NM_{—}007175) is another VGAM312 host target gene. C8orf2 BINDING SITE is HOST TARGET binding site found in the 3′ untranslated region of mRNA encoded by C8orf2, corresponding to a HOST TARGET binding site such as BINDING SITE I, BINDING SITE II or BINDING SITE III. Table 2 illustrates the complementarity of the nucleotide sequences of C8orf2 BINDING SITE, designated SEQ ID:1361, to the nucleotide sequence of VGAM312 RNA, herein designated VGAM RNA, also designated SEQ ID:647.

Another function of VGAM312 is therefore inhibition of Chromosome 8 Open Reading Frame 2 (C8orf2, Accession NM_{—}007175). Accordingly, utilities of VGAM312 include diagnosis, prevention and treatment of diseases and clinical conditions associated with C8orf2. DKFZP566K0524 (Accession XM_{—}045128) is another VGAM312 host target gene. DKFZP566K0524 BINDING SITE is HOST TARGET binding site found in the 3′ untranslated region of mRNA encoded by DKFZP566K0524, corresponding to a HOST TARGET binding site such as BINDING SITE I, BINDING SITE II or BINDING SITE III. Table 2 illustrates the complementarity of the nucleotide sequences of DKFZP566K0524 BINDING SITE, designated SEQ ID:2854, to the nucleotide sequence of VGAM312 RNA, herein designated VGAM RNA, also designated SEQ ID:647.

Another function of VGAM312 is therefore inhibition of DKFZP566K0524 (Accession XM_{—}045128). Accordingly, utilities of VGAM312 include diagnosis, prevention and treatment of diseases and clinical conditions associated with DKFZP566K0524. Fidgetin-like 1 (FIGNL1, Accession NM_{—}022116) is another VGAM312 host target gene. FIGNL1 BINDING SITE is HOST TARGET binding site found in the 3′ untranslated region of mRNA encoded by FIGNL1, corresponding to a HOST TARGET binding site such as BINDING SITE I, BINDING SITE II or BINDING SITE III. Table 2 illustrates the complementarity of the nucleotide sequences of FIGNL1 BINDING SITE, designated SEQ ID:1980, to the nucleotide sequence of VGAM312 RNA, herein designated VGAM RNA, also designated SEQ ID:647.

Another function of VGAM312 is therefore inhibition of Fidgetin-like 1 (FIGNL1, Accession NM_{—}022116). Accordingly, utilities of VGAM312 include diagnosis, prevention and treatment of diseases and clinical conditions associated with FIGNL1. FLJ10704 (Accession NM_{—}018185) is another VGAM312 host target gene. FLJ10704 BINDING SITE is HOST TARGET binding site found in the 3′ untranslated region of mRNA encoded by FLJ10704, corresponding to a HOST TARGET binding site such as BINDING SITE I, BINDING SITE II or BINDING SITE III. Table 2 illustrates the complementarity of the nucleotide sequences of FLJ10704 BINDING SITE, designated SEQ ID:1796, to the nucleotide sequence of VGAM312 RNA, herein designated VGAM RNA, also designated SEQ ID:647.

Another function of VGAM312 is therefore inhibition of FLJ10704 (Accession NM_{—}018185). Accordingly, utilities of VGAM312 include diagnosis, prevention and treatment of diseases and clinical conditions associated with FLJ10704. HDAC9-PENDING (Accession NM_{—}014707) is another VGAM312 host target gene. HDAC9-PENDING BINDING SITE is HOST TARGET binding site found in the 3′ untranslated region of mRNA encoded by HDAC9-PENDING, corresponding to a HOST TARGET binding site such as BINDING SITE I, BINDING SITE II or BINDING SITE III. Table 2 illustrates the complementarity of the nucleotide sequences of HDAC9-PENDING BINDING SITE, designated SEQ ID: 1528, to the nucleotide sequence of VGAM312 RNA, herein designated VGAM RNA, also designated SEQ ID:647.

Another function of VGAM312 is therefore inhibition of HDAC9-PENDING (Accession NM_{—}014707). Accordingly, utilities of VGAM312 include diagnosis, prevention and treatment of diseases and clinical conditions associated with HDAC9-PENDING. KIAA1384 (Accession XM_{—}035405) is another VGAM312 host target gene. KIAA1384 BINDING SITE is HOST TARGET binding site found in the 3′ untranslated region of mRNA encoded by KIAA1384, corresponding to a HOST TARGET binding site such as BINDING SITE I, BINDING SITE II or BINDING SITE III. Table 2 illustrates the complementarity of the nucleotide sequences of KIAA1384 BINDING SITE, designated SEQ ID:2695, to the nucleotide sequence of VGAM312 RNA, herein designated VGAM RNA, also designated SEQ ID:647.

Another function of VGAM312 is therefore inhibition of KIAA1384 (Accession XM_{—}035405). Accordingly, utilities of VGAM312 include diagnosis, prevention and treatment of diseases and clinical conditions associated with KIAA1384. SOX30 (Accession NM_{—}007017) is another VGAM312 host target gene. SOX30 BINDING SITE is HOST TARGET binding site found in the 3′ untranslated region of mRNA encoded by SOX30, corresponding to a HOST TARGET binding site such as BINDING SITE I, BINDING SITE II or BINDING SITE III. Table 2 illustrates the complementarity of the nucleotide sequences of SOX30 BINDING SITE, designated SEQ ID:1346, to the nucleotide sequence of VGAM312 RNA, herein designated VGAM RNA, also designated SEQ ID:647.

Another function of VGAM312 is therefore inhibition of SOX30 (Accession NM_{—}007017). Accordingly, utilities of VGAM312 include diagnosis, prevention and treatment of diseases and clinical conditions associated with SOX30. LOC221810 (Accession XM_{—}168222) is another VGAM312 host target gene. LOC221810 BINDING SITE is HOST TARGET binding site found in the 3′ untranslated region of mRNA encoded by LOC221810, corresponding to a HOST TARGET binding site such as BINDING SITE I, BINDING SITE II or BINDING SITE III. Table 2 illustrates the complementarity of the nucleotide sequences of LOC221810 BINDING SITE, designated SEQ ID:3625, to the nucleotide sequence of VGAM312 RNA, herein designated VGAM RNA, also designated SEQ ID:647.

Another function of VGAM312 is therefore inhibition of LOC221810 (Accession XM_{—}168222). Accordingly, utilities of VGAM312 include diagnosis, prevention and treatment of diseases and clinical conditions associated with LOC221810. LOC255177 (Accession XM_{—}172941) is another VGAM312 host target gene. LOC255177 BINDING SITE is HOST TARGET binding site found in the 3′ untranslated region of mRNA encoded by LOC255177, corresponding to a HOST TARGET binding site such as BINDING SITE I, BINDING SITE II or BINDING SITE III. Table 2 illustrates the complementarity of the nucleotide sequences of LOC255177 BINDING SITE, designated SEQ ID:3712, to the nucleotide sequence of VGAM312 RNA, herein designated VGAM RNA, also designated SEQ ID:647.

Another function of VGAM312 is therefore inhibition of LOC255177 (Accession XM_{—}172941). Accordingly, utilities of VGAM312 include diagnosis, prevention and treatment of diseases and clinical conditions associated with LOC255177. FIG. 1 further provides a conceptual description of a novel bioinformatically detected viral gene of the present invention, referred to here as Viral Genomic Address Messenger 313 (VGAM313) viral gene, which modulates expression of respective host target genes thereof, the function and utility of which host target genes is known in the art.

VGAM313 is a novel bioinformatically detected regulatory, non protein coding, viral micro RNA (miRNA) gene. The method by which VGAM313 was detected is described hereinabove with reference to FIGS. 1-8.

VGAM313 gene, herein designated VGAM GENE, is a viral gene contained in the genome of Vaccinia Virus. VGAM313 host target gene, herein designated VGAM HOST TARGET GENE, is a human gene contained in the human genome.

VGAM313 gene encodes a VGAM313 precursor RNA, herein designated VGAM PRECURSOR RNA. Similar to other miRNA genes, and unlike most ordinary genes, VGAM313 precursor RNA does not encode a protein. A nucleotide sequence identical or highly similar to the nucleotide sequence of VGAM313 precursor RNA is designated SEQ ID:299, and is provided hereinbelow with reference to the sequence listing part. Nucleotide sequence SEQ ID:299 is located at position 10272 relative to the genome of Vaccinia Virus.

VGAM313 precursor RNA folds onto itself, forming VGAM313 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM313 folded precursor RNA into VGAM313 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM313 RNA is designated SEQ ID:648, and is provided hereinbelow with reference to the sequence listing part.

VGAM313 host target gene, herein designated VGAM HOST TARGET GENE, encodes a corresponding messenger RNA, VGAM313 host target RNA, herein designated VGAM HOST TARGET RNA. VGAM313 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM313 RNA, herein designated VGAM RNA, binds complementarily to one or more host target binding sites located in untranslated regions of VGAM313 host target RNA, herein designated VGAM HOST TARGET RNA. This complementary binding is due to the fact that the nucleotide sequence of VGAM313 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM313 RNA, herein designated VGAM RNA, may have a different number of host target binding sites in untranslated regions of a VGAM313 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM313 RNA, herein designated VGAM RNA, to host target binding sites on VGAM313 host target RNA, herein designated VGAM HOST TARGET RNA, such as BINDING SITE I, BINDING SITE II and BINDING SITE III, inhibits translation of VGAM313 host target RNA into VGAM313 host target protein, herein designated VGAM HOST TARGET PROTEIN. VGAM host target protein is therefore outlined by a broken line.

It is appreciated that VGAM313 host target gene, herein designated VGAM HOST TARGET GENE, in fact represents a plurality of VGAM313 host target genes. The mRNA of each one of this plurality of VGAM313 host target genes comprises one or more host target binding sites, each having a nucleotide sequence which is at least partly complementary to VGAM313 RNA, herein designated VGAM RNA, and which when bound by VGAM313 RNA causes inhibition of translation of respective one or more VGAM313 host target proteins.

It is further appreciated by one skilled in the art that the mode of translational inhibition illustrated by FIG. 1 with specific reference to translational inhibition exerted by VGAM313 gene, herein designated VGAM GENE, on one or more VGAM313 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM313 is inhibition of expression of host target genes, as part of a novel viral mechanism of attacking a host. Accordingly, utilities of VGAM313 include diagnosis, prevention and treatment of viral infection by Vaccinia Virus. Specific functions, and accordingly utilities, of VGAM313 correlate with, and may be deduced from, the identity of the host target genes which VGAM313 binds and inhibits, and the function of these host target genes, as elaborated hereinbelow.

Nucleotide sequences of the VGAM313 precursor RNA, herein designated VGAM PRECURSOR RNA, and of the ‘diced’ VGAM313 RNA, herein designated VGAM RNA, and a schematic representation of the secondary folding of VGAM313 folded precursor RNA, herein designated VGAM FOLDED PRECURSOR RNA, of VGAM313 are further described hereinbelow with reference to Table 1.

Nucleotide sequences of host target binding sites, such as BINDING SITE-I, BINDING SITE-II and BINDING SITE-III of FIG. 1, found on VGAM313 host target RNA, and schematic representation of the complementarity of each of these host target binding sites to VGAM313 RNA, herein designated VGAM RNA, are described hereinbelow with reference to Table 2.

As mentioned hereinabove with reference to FIG. 1, a function of VGAM313 gene, herein designated VGAM is inhibition of expression of VGAM313 target genes. It is appreciated that specific functions, and accordingly utilities, of VGAM313 correlate with, and may be deduced from, the identity of the target genes which VGAM313 binds and inhibits, and the function of these target genes, as elaborated hereinbelow.

Adrenergic, Alpha-1A-, Receptor (ADRA1A, Accession NM_{—}033303) is a VGAM313 host target gene. ADRA1A BINDING SITE is HOST TARGET binding site found in the 3′ untranslated region of mRNA encoded by ADRA1A, corresponding to a HOST TARGET binding site such as BINDING SITE I, BINDING SITE II or BINDING SITE III. Table 2 illustrates the complementarity of the nucleotide sequences of ADRA1A BINDING SITE, designated SEQ ID:2327, to the nucleotide sequence of VGAM313 RNA, herein designated VGAM RNA, also designated SEQ ID:648.

A function of VGAM313 is therefore inhibition of Adrenergic, Alpha-1A-, Receptor (ADRA1A, Accession NM_{—}033303). Accordingly, utilities of VGAM313 include diagnosis, prevention and treatment of diseases and clinical conditions associated with ADRA1A. ADP-ribosylation Factor 3 (ARF3, Accession NM_{—}001659) is another VGAM313 host target gene. ARF3 BINDING SITE is HOST TARGET binding site found in the 3′ untranslated region of mRNA encoded by ARF3, corresponding to a HOST TARGET binding site such as BINDING SITE I, BINDING SITE II or BINDING SITE III. Table 2 illustrates the complementarity of the nucleotide sequences of ARF3 BINDING SITE, designated SEQ ID:845, to the nucleotide sequence of VGAM313 RNA, herein designated VGAM RNA, also designated SEQ ID:648.

Another function of VGAM313 is therefore inhibition of ADP-ribosylation Factor 3 (ARF3, Accession NM_{—}001659). Accordingly, utilities of VGAM313 include diagnosis, prevention and treatment of diseases and clinical conditions associated with ARF3. Chromosome 14 Open Reading Frame 1 (C14orf1, Accession NM_{—}007176) is another VGAM313 host target gene. C14orf1 BINDING SITE is HOST TARGET binding site found in the 3′ untranslated region of mRNA encoded by C14orf1, corresponding to a HOST TARGET binding site such as BINDING SITE I, BINDING SITE II or BINDING SITE III. Table 2 illustrates the complementarity of the nucleotide sequences of C14orf1 BINDING SITE, designated SEQ ID:1362, to the nucleotide sequence of VGAM313 RNA, herein designated VGAM RNA, also designated SEQ ID:648.

Another function of VGAM313 is therefore inhibition of Chromosome 14 Open Reading Frame 1 (C14orf1, Accession NM_{—}007176). Accordingly, utilities of VGAM313 include diagnosis, prevention and treatment of diseases and clinical conditions associated with C14orf1. Catenin (cadherin-associated protein), Alpha 2 (CTNNA2, Accession NM_{—}004389) is another VGAM313 host target gene. CTNNA2 BINDING SITE is HOST TARGET binding site found in the 3′ untranslated region of mRNA encoded by CTNNA2, corresponding to a HOST TARGET binding site such as BINDING SITE I, BINDING SITE II or BINDING SITE III. Table 2 illustrates the complementarity of the nucleotide sequences of CTNNA2 BINDING SITE, designated SEQ ID:1108, to the nucleotide sequence of VGAM313 RNA, herein designated VGAM RNA, also designated SEQ ID:648.

Another function of VGAM313 is therefore inhibition of Catenin (cadherin-associated protein), Alpha 2 (CTNNA2, Accession NM_{—}004389), a gene which is involved in the cytoplasmic anchorage of cell-cell and cell-substrate adhesion molecules. Accordingly, utilities of VGAM313 include diagnosis, prevention and treatment of diseases and clinical conditions associated with CTNNA2. The function of CTNNA2 has been established by previous studies. Cell-cell and cell-matrix adhesions involve transmembrane glycoproteins such as cell adhesion molecules and integrins, which are thought to function via interactions of their cytoplasmic domains with proteins associated with the cytoskeleton. Vinculin (OMIM Ref. No. 193065) and talin (OMIM Ref. No. 186745) are examples. The activity of cadherins (e.g., 114020), which mediate homophilic cell-cell Ca(2+)-dependent association, depends on their anchorage to cytoskeleton via proteins termed catenins (Herrenknecht et al., 1991). Animal model experiments lend further support to the function of CTNNA2. Mice homozygous for the ‘cerebellar-deficient folia’ (cdf) mutation are ataxic and have cerebellar hypoplasia and abnormal lobulation of the cerebellum (Cook et al., 1997). In the cerebella of cdf/cdf homozygous mice, approximately 40% of Purkinje cells are located ectopically in the white matter and inner granule-cell layer. Many hippocampal pyramidal cells are scattered in the plexiform layers, and those that are correctly positioned are less densely packed than are cells in wildtype mice. Park et al. (2002) showed that fear conditioning and prepulse inhibition of the startle response are also disrupted in cdf/cdf mice. They identified a deletion on mouse chromosome 6 that removed approximately 150 kb of the cdf region. The deletion included part of Catna2, encoding alpha-N-catenin, a protein that links the classic cadherins to the neuronal cytoskeleton. Expression of a Catna2 transgene in cdf/cdf mice restored normal cerebellar and hippocampal morphology, prepulse inhibition, and fear conditioning. The findings suggested that catenin-cadherin cell-adhesion complexes are important in cerebellar and hippocampal lamination and in the control of startle modulation.

It is appreciated that the abovementioned animal model for CTNNA2 is acknowledged by those skilled in the art as a scientifically valid animal model, as can be further appreciated from the publications sited hereinbelow.

Full details of the abovementioned studies are described in the following publications, the disclosure of which are hereby incorporated by reference:
Clayerie, J.-M.; Hardelin, J.-P.; Legouis, R.; Levilliers, J.; Bougueleret, L.; Mattei, M.-G.; Petit, C.: Characterization and chromosomal assignment of a human cDNA encoding a protein related to the murine 102-kDa cadherin-associated protein (alpha-catenin). Genomics 15: 13-20, 1993.; and
Park, C.; Falls, W.; Finger, J. H.; Longo-Guess, C. M.; Ackerman, S. L.: Deletion in Catna2, encoding alpha-N-catenin, causes cerebellar and hippocampal lamination defects and impaired.

Further studies establishing the function and utilities of CTNNA2 are found in John Hopkins OMIM database record ID 114025, and in sited publications numbered 2682-2683, 220 and 2684-2685 listed in the bibliography section hereinbelow, which are also hereby incorporated by reference. FAT Tumor Suppressor Homolog 2 (Drosophila) (FAT2, Accession NM_{—}001447) is another VGAM313 host target gene. FAT2 BINDING SITE is HOST TARGET binding site found in the 3′ untranslated region of mRNA encoded by FAT2, corresponding to a HOST TARGET binding site such as BINDING SITE I, BINDING SITE II or BINDING SITE III. Table 2 illustrates the complementarity of the nucleotide sequences of FAT2 BINDING SITE, designated SEQ ID:829, to the nucleotide sequence of VGAM313 RNA, herein designated VGAM RNA, also designated SEQ ID:648.

Another function of VGAM313 is therefore inhibition of FAT Tumor Suppressor Homolog 2 (Drosophila) (FAT2, Accession NM_{—}001447), a gene which could function as a cell-adhesion protein. Accordingly, utilities of VGAM313 include diagnosis, prevention and treatment of diseases and clinical conditions associated with FAT2. The function of FAT2 has been established by previous studies. The domain that characterizes epidermal growth factor (EGF; 131530) consists of approximately 50 amino acids with 3 disulfide bonds. EGF-like domains are believed to play a critical role in a number of extracellular events, including cell adhesion and receptor-ligand interactions. Proteins with EGF-like domains often consist of more than 1,000 amino acids, have multiple copies of the EGF-like domain, and contain additional domains known to be involved in specific protein-protein interactions. To identify proteins containing EGF-like domains, Nakayama et al. (1998) searched a database of long cDNA sequences randomly selected from a human brain cDNA library for those that encode an EGF-like motif. They identified several partial cDNAs encoding novel proteins with EGF-like domains, such as FAT2, which they named MEGF1. Nakayama et al. (1998) isolated a rat cDNA containing the complete Megf1 coding sequence. The predicted Megf1 protein has a signal sequence, 34 cadherin motifs (see OMIM Ref. No. 603006), a laminin G domain (see OMIM Ref. No. 601033), 2 EGF-like domains, a transmembrane domain, a cytoplasmic proline-rich sequence, and a cytoplasmic RGD (arginine-glycine-aspartic acid) motif, which is found in proteins modulating cell adhesion. The predicted structure of Megf1 is similar overall to the structures of the Drosophila ‘fat’ gene product and human FAT (OMIM Ref. No. 600976), although the number of EGF-like domains varies among these proteins. The Drosophila fat gene is a tumor suppressor gene whose product controls cell proliferation and morphogenesis in the imaginal discs in a contact-dependent manner. Northern blot analysis of various regions of rat brain detected Megf1 expression only in the cerebellum.

Full details of the abovementioned studies are described in the following publications, the disclosure of which are hereby incorporated by reference:
Nakayama, M.; Nakajima, D.; Nagase, T.; Nomura, N.; Seki, N.; Ohara, O.: Identification of high-molecular-weight proteins with multiple EGF-like motifs by motif-trap screening. Genomics 51: 27-34, 1998.; and
Wu, Q.; Maniatis, T.: Large exons encoding multiple ectodomains are a characteristic feature of protocadherin genes. Proc. Nat. Acad. Sci. 97: 3124-3129, 2000.

Further studies establishing the function and utilities of FAT2 are found in John Hopkins OMIM database record ID 604269, and in sited publications numbered 1672-1673 listed in the bibliography section hereinbelow, which are also hereby incorporated by reference. UDP-N-acetyl-alpha-D-galactosamine: polypeptide N-acetylgalactosaminyltransferase 3 (Gal NAc-T3) (GALNT3, Accession NM_{—}004482) is another VGAM313 host target gene. GALNT3 BINDING SITE is HOST TARGET binding site found in the 3′ untranslated region of mRNA encoded by GALNT3, corresponding to a HOST TARGET binding site such as BINDING SITE I, BINDING SITE II or BINDING SITE III. Table 2 illustrates the complementarity of the nucleotide sequences of GALNT3 BINDING SITE, designated SEQ ID:1118, to the nucleotide sequence of VGAM313 RNA, herein designated VGAM RNA, also designated SEQ ID:648.

Another function of VGAM313 is therefore inhibition of UDP-N-acetyl-alpha-D-galactosamine: polypeptide N-acetylgalactosaminyltransferase 3 (GalNAc-T3) (GALNT3, Accession NM_{—}004482), a gene which initiates O-glycosylation of serine and threonine residues. Accordingly, utilities of VGAM313 include diagnosis, prevention and treatment of diseases and clinical conditions associated with GALNT3. The function of GALNT3 has been established by previous studies. GALNT3 (EC 2.4.1.41) is one of several enzymes that catalyze the reaction UDP-Gal NAc+polypeptide-(Ser/Thr)-OH to GalNAc-alpha-O-Ser/Thr-polypeptide+UDP, thereby initiating O-glycosylation of serine and threonine residues on an array of glycoproteins. Bennett et al. (1996) used degenerate PCR to clone human GALNT3 using primers based on the sequences of GALNT1 (OMIM Ref. No. 602273) and GALNT2 (OMIM Ref. No. 602274). GALNT3 encodes a 633-amino acid protein which has a single membrane-spanning region and is highly homologous to GALNT1 and GALNT2. Northern blot analysis showed that GALNT3 is expressed as a 3.6-kb transcript, with highest levels in human pancreas and testis. Bennett et al. (1996) expressed the gene in insect Sf9 cells and showed that GALNT3 does have GalNAc-transferase activity, but with different substrate specificity than GALNT1 or GALNT2. The mouse ortholog of GalNAc-T3 was cloned by Zara et al. (1996). Bennett et al. (1998) found that the GALNT1, GALNT2, and GALNT3 genes contain 11, 16, and 10 exons, respectively. Several intron/exon boundaries are conserved within the 3 genes. By FISH, Bennett et al. (1998) mapped the GALNT3 gene to human chromosome 2q24-q31.

Full details of the abovementioned studies are described in the following publications, the disclosure of which are hereby incorporated by reference:
Bennett, E. P.; Hassan, H.; Clausen, H.: cDNA cloning and expression of a novel human UDPN-acetyl-alpha-D-galactosamine. J. Biol. Chem. 271: 17006-17012, 1996.; and
Bennett, E. P.; Weghuis, D. O.; Merkx, G.; Geurts van Kessel, A.; Eiberg, H.; Clausen, H.: Genomic organization and chromosomal localization of three members of the UDP-N-acetylgalacto.

Further studies establishing the function and utilities of GALNT3 are found in John Hopkins OMIM database record ID 601756, and in sited publications numbered 647-649 listed in the bibliography section hereinbelow, which are also hereby incorporated by reference. High-mobility Group 20A (HMG20A, Accession NM_{—}018200) is another VGAM313 host target gene. HMG20A BINDING SITE is HOST TARGET binding site found in the 3′ untranslated region of mRNA encoded by HMG20A, corresponding to a HOST TARGET binding site such as BINDING SITE I, BINDING SITE II or BINDING SITE III. Table 2 illustrates the complementarity of the nucleotide sequences of HMG20A BINDING SITE, designated SEQ ID:1801, to the nucleotide sequence of VGAM313 RNA, herein designated VGAM RNA, also designated SEQ ID:648.

Another function of VGAM313 is therefore inhibition of High-mobility Group 20A (HMG20A, Accession NM_{—}018200). Accordingly, utilities of VGAM313 include diagnosis, prevention and treatment of diseases and clinical conditions associated with HMG20A. Hippocalcin (HPCA, Accession NM_{—}002143) is another VGAM313 host target gene. HPCA BINDING SITE is HOST TARGET binding site found in the 3′ untranslated region of mRNA encoded by HPCA, corresponding to a HOST TARGET binding site such as BINDING SITE I, BINDING SITE II or BINDING SITE III. Table 2 illustrates the complementarity of the nucleotide sequences of HPCA BINDING SITE, designated SEQ ID:903, to the nucleotide sequence of VGAM313 RNA, herein designated VGAM RNA, also designated SEQ ID:648.

Another function of VGAM313 is therefore inhibition of Hippocalcin (HPCA, Accession NM_{—}002143), a gene which may be an hippocampal calcium-binding protein. Accordingly, utilities of VGAM313 include diagnosis, prevention and treatment of diseases and clinical conditions associated with HPCA. The function of HPCA has been established by previous studies. Hippocalcin is a member of a family of neuron-specific Ca(2+)-binding proteins found in the retina and brain. Hippocalcin is a 23-kD Ca(2+)-binding protein first identified in the rat hippocampus (Kobayashi et al., 1992). The primary structure of rat hippocalcin comprises 193 amino acid residues and shows striking sequence similarities to proteins located in the photo receptor cells that regulate photosignal transduction in a Ca(2+)-sensitive manner. Hippocalcin is associated with the plasma membrane. Takamatsu et al. (1994) isolated a cDNA clone encoding human hippocalcin from a human hippocampus cDNA library. The human sequence showed 100% amino acid identity with the rat sequence and 92% nucleotide identity. Northern blot analysis detected a single 2.0-kb HPCA transcript only in brain.

Full details of the abovementioned studies are described in the following publications, the disclosure of which are hereby incorporated by reference:
Kobayashi, M.; Takamatsu, K.; Saitoh, S.; Miura, M.; Noguchi, T.: Molecular cloning of hippocalcin, a novel calcium-binding protein of the recover in family exclusively expressed in hippocampus. Biochem. Biophys. Res. Commun. 189: 511-517, 1992.; and
Takamatsu, K.; Kobayashi, M.; Saitoh, S.; Fujishiro, M.; Noguchi, T.: Molecular cloning of human hippocalcin cDNA and chromosomal mapping of its gene. Biochem. Biophys. Res. Commun. 20.

Further studies establishing the function and utilities of HPCA are found in John Hopkins OMIM database record ID 142622, and in sited publications numbered 890-891 listed in the bibliography section hereinbelow, which are also hereby incorporated by reference. Myotubularin Related Protein 3 (MTMR3, Accession NM_{—}021090) is another VGAM313 host target gene. MTMR3 BINDING SITE is HOST TARGET binding site found in the 3′ untranslated region of mRNA encoded by MTMR3, corresponding to a HOST TARGET binding site such as BINDING SITE I, BINDING SITE II or BINDING SITE III. Table 2 illustrates the complementarity of the nucleotide sequences of MTMR3 BINDING SITE, designated SEQ ID:1939, to the nucleotide sequence of VGAM313 RNA, herein designated VGAM RNA, also designated SEQ ID:648.

Another function of VGAM313 is therefore inhibition of Myotubularin Related Protein 3 (MTMR3, Accession NM_{—}021090), a gene which could be a tyrosine-phosphatase. Accordingly, utilities of VGAM313 include diagnosis, prevention and treatment of diseases and clinical conditions associated with MTMR3. The function of MTMR3 has been established by previous studies. Zhao et al. (2001) showed that an isoform of MTMR3, missing exon 17, dephosphorylates para-nitrophenylphosphate and phosphatidylinositol 3-phosphate.

Full details of the abovementioned studies are described in the following publications, the disclosure of which are hereby incorporated by reference:
Nagase, T.; Ishikawa, K.; Nakajima, D.; Ohira, M.; Seki, N.; Miyajima, N.; Tanaka, A.; Kotani, H.; Nomura, N.; Ohara, O.: Prediction of the coding sequences of unidentified human genes. VII. The complete sequences of 100 new cDNA clones from brain which can code for large proteins in vitro. DNA Res. 4: 141-150, 1997.; and
Zhao, R.; Qi, Y.; Chen, J.; Zhao, Z. J.: FYVE-DSP2, a FYVE domain-containing dual specificity protein phosphatase that dephosphorylates phosphotidylinositol (sic) 3-phosphate. Exp. Cel.

Further studies establishing the function and utilities of MTMR3 are found in John Hopkins OMIM database record ID 603558, and in sited publications numbered 164 and 1133 listed in the bibliography section hereinbelow, which are also hereby incorporated by reference. Polymeric Immunoglobulin Receptor (PIGR, Accession XM_{—}052013) is another VGAM313 host target gene. PIGR BINDING SITE is HOST TARGET binding site found in the 3′ untranslated region of mRNA encoded by PIGR, corresponding to a HOST TARGET binding site such as BINDING SITE I, BINDING SITE II or BINDING SITE III. Table 2 illustrates the complementarity of the nucleotide sequences of PIGR BINDING SITE, designated SEQ ID:2955, to the nucleotide sequence of VGAM313 RNA, herein designated VGAM RNA, also designated SEQ ID:648.

Another function of VGAM313 is therefore inhibition of Polymeric Immunoglobulin Receptor (PIGR, Accession XM_{—}052013). Accordingly, utilities of VGAM313 include diagnosis, prevention and treatment of diseases and clinical conditions associated with PIGR. Piwi-like 1 (Drosophila) (PIWIL1, Accession XM_{—}052767) is another VGAM313 host target gene. PIWIL1BINDING SITE is HOST TARGET binding site found in the 3′ untranslated region of mRNA encoded by PIWIL1, corresponding to a HOST TARGET binding site such as BINDING SITE I, BINDING SITE II or BINDING SITE III. Table 2 illustrates the complementarity of the nucleotide sequences of PIWIL1BINDING SITE, designated SEQ ID:2959, to the nucleotide sequence of VGAM313 RNA, herein designated VGAM RNA, also designated SEQ ID:648.

Another function of VGAM313 is therefore inhibition of Piwi-like 1 (Drosophila) (PIWIL1, Accession XM_{—}052767). Accordingly, utilities of VGAM313 include diagnosis, prevention and treatment of diseases and clinical conditions associated with PIWIL1. Sorbin and SH3 Domain Containing 1 (SORBS1, Accession NM_{—}015385) is another VGAM313 host target gene. SORBS1 BINDING SITE is HOST TARGET binding site found in the 3′ untranslated region of mRNA encoded by SORBS1, corresponding to a HOST TARGET binding site such as BINDING SITE I, BINDING SITE II or BINDING SITE III. Table 2 illustrates the complementarity of the nucleotide sequences of SORBS1 BINDING SITE, designated SEQ ID:1625, to the nucleotide sequence of VGAM313 RNA, herein designated VGAM RNA, also designated SEQ ID:648.

Another function of VGAM313 is therefore inhibition of Sorbin and SH3 Domain Containing 1 (SORBS1, Accession NM_{—}015385), a gene which necessary for cell polarization during vegetative growth. Accordingly, utilities of VGAM313 include diagnosis, prevention and treatment of diseases and clinical conditions associated with SORBS1. The function of SORBS1 has been established by previous studies. Lin et al. (2001) identified 14 single-nucleotide polymorphisms (SNPs) in the human SH3P12 gene, which they called SORBS1. Studies in 202 nonobese, 113 obese, and 455 subjects with type II diabetes (OMIM Ref. No. 125853) revealed that the alanine allele of a T228A polymorphism in exon 7 exerted a protective role for both obesity (OMIM Ref. No. 601665) (relative risk 0.466; 95% confidence interval 0.265 to 0.821) and diabetes (relative risk 0.668; 95% confidence interval 0.472 to 0.945). Neither allele of the R74W polymorphism was associated with either obesity or diabetes. The authors suggested that the SH3P12 gene may play an important role in the pathogenesis of human disorders with insulin resistance. Insulin stimulates the transport of glucose into fat and muscle cells and initiates its actions by binding to its tyrosine kinase receptor, leading to the phosphorylation of intracellular substrates. One such substrate is the CBL protooncogene product. CBL is recruited to the insulin receptor by interaction with the adaptor protein CAP, through 1 of 3 adjacent SH3 domains in the C terminus of CAP. Upon phosphorylation of CBL, the CAP-CBL complex dissociates from the insulin receptor and moves to a caveolin (see OMIM Ref. No. 601047)-enriched triton-insoluble membrane fraction (Mastick et al., 1995). To identify a molecular mechanism underlying this subcellular redistribution, Baumann et al. (2000) screened a yeast 2-hybrid library using the N-terminal region of CAP and identified the caveolar protein flotillin (OMIM Ref. No. 131560). Flotillin forms a ternary complex with CAP and CBL, directing the localization of the CAP-CBL complex to a lipid raft subdomain of the plasma membrane. Expression of the N-terminal domain of CAP in 3T3-L1 adipocytes blocks the stimulation of glucose transport by insulin, without affecting signaling events that depend on phosphatidylinositol-3-OH kinase (see OMIM Ref. No. 602838). Thus, localization of the CBL-CAP complex to lipid rafts generates a pathway that is crucial in the regulation of glucose uptake.

Full details of the abovementioned studies are described in the following publications, the disclosure of which are hereby incorporated by reference:
Lin, W.-H.; Chiu, K. C.; Chang, H.-M.; Lee, K.-C.; Tai, T.-Y.; Chuang, L.-M.: Molecular scanning of the human sorbin and SH3-domain-containing-1 (SORBS1) gene: positive association of the T228A polymorphism with obesity and type 2 diabetes. Hum. Molec. Genet. 10: 1753-1760, 2001.; and
Baumann, C. A.; Ribon, V.; Kanzaki, M.; Thurmond, D. C.; Mora, S.; Shigematsu, S.; Bickel, P. E.; Pessin, J. E.; Saltiel, A. R.: CAP defines a second signalling pathway required for insul.

Further studies establishing the function and utilities of SORBS1 are found in John Hopkins OMIM database record ID 605264, and in sited publications numbered 1046, 2780-1141, 250 and 2786 listed in the bibliography section hereinbelow, which are also hereby incorporated by reference. Topoisomerase (DNA) III Alpha (TOP3A, Accession NM_{—}004618) is another VGAM313 host target gene. TOP3A BINDING SITE is HOST TARGET binding site found in the 3′ untranslated region of mRNA encoded by TOP3A, corresponding to a HOST TARGET binding site such as BINDING SITE I, BINDING SITE II or BINDING SITE III. Table 2 illustrates the complementarity of the nucleotide sequences of TOP3A BINDING SITE, designated SEQ ID:1132, to the nucleotide sequence of VGAM313 RNA, herein designated VGAM RNA, also designated SEQ ID:648.

Another function of VGAM313 is therefore inhibition of Topoisomerase (DNA) III Alpha (TOP3A, Accession NM_{—}004618). Accordingly, utilities of VGAM313 include diagnosis, prevention and treatment of diseases and clinical conditions associated with TOP3A. UC28 (Accession NM_{—}021635) is another VGAM313 host target gene. UC28 BINDING SITE is HOST TARGET binding site found in the 3′ untranslated region of mRNA encoded by UC28, corresponding to a HOST TARGET binding site such as BINDING SITE I, BINDING SITE II or BINDING SITE III. Table 2 illustrates the complementarity of the nucleotide sequences of UC28 BINDING SITE, designated SEQ ID:1951, to the nucleotide sequence of VGAM313 RNA, herein designated VGAM RNA, also designated SEQ ID:648.

Another function of VGAM313 is therefore inhibition of UC28 (Accession NM_{—}021635). Accordingly, utilities of VGAM313 include diagnosis, prevention and treatment of diseases and clinical conditions associated with UC28. ADP-ribosylation Factor-like 5 (ARL5, Accession NM_{—}012097) is another VGAM313 host target gene. ARL5 BINDING SITE is HOST TARGET binding site found in the 3′ untranslated region of mRNA encoded by ARL5, corresponding to a HOST TARGET binding site such as BINDING SITE I, BINDING SITE II or BINDING SITE III. Table 2 illustrates the complementarity of the nucleotide sequences of ARL5 BINDING SITE, designated SEQ ID: 1406, to the nucleotide sequence of VGAM313 RNA, herein designated VGAM RNA, also designated SEQ ID:648.

Another function of VGAM313 is therefore inhibition of ADP-ribosylation Factor-like 5 (ARL5, Accession NM_{—}012097). Accordingly, utilities of VGAM313 include diagnosis, prevention and treatment of diseases and clinical conditions associated with ARL5. DDM36 (Accession NM_{—}020962) is another VGAM313 host target gene. DDM36 BINDING SITE is HOST TARGET binding site found in the 3′ untranslated region of mRNA encoded by DDM36, corresponding to a HOST TARGET binding site such as BINDING SITE I, BINDING SITE II or BINDING SITE III. Table 2 illustrates the complementarity of the nucleotide sequences of DDM36 BINDING SITE, designated SEQ ID:1932, to the nucleotide sequence of VGAM313 RNA, herein designated VGAM RNA, also designated SEQ ID:648.

Another function of VGAM313 is therefore inhibition of DDM36 (Accession NM_{—}020962). Accordingly, utilities of VGAM313 include diagnosis, prevention and treatment of diseases and clinical conditions associated with DDM36. FLJ21596 (Accession NM_{—}024823) is another VGAM313 host target gene. FLJ21596 BINDING SITE is HOST TARGET binding site found in the 3′ untranslated region of mRNA encoded by FLJ21596, corresponding to a HOST TARGET binding site such as BINDING SITE I, BINDING SITE II or BINDING SITE III. Table 2 illustrates the complementarity of the nucleotide sequences of FLJ21596 BINDING SITE, designated SEQ ID:2093, to the nucleotide sequence of VGAM313 RNA, herein designated VGAM RNA, also designated SEQ ID:648.

Another function of VGAM313 is therefore inhibition of FLJ21596 (Accession NM_{—}024823). Accordingly, utilities of VGAM313 include diagnosis, prevention and treatment of diseases and clinical conditions associated with FLJ21596. FLJ22202 (Accession NM_{—}024883) is another VGAM313 host target gene. FLJ22202 BINDING SITE is HOST TARGET binding site found in the 5′ untranslated region of mRNA encoded by FLJ22202, corresponding to a HOST TARGET binding site such as BINDING SITE I, BINDING SITE II or BINDING SITE III. Table 2 illustrates the complementarity of the nucleotide sequences of FLJ22202 BINDING SITE, designated SEQ ID:2102, to the nucleotide sequence of VGAM313 RNA, herein designated VGAM RNA, also designated SEQ ID:648.

Another function of VGAM313 is therefore inhibition of FLJ22202 (Accession NM_{—}024883). Accordingly, utilities of VGAM313 include diagnosis, prevention and treatment of diseases and clinical conditions associated with FLJ22202. Glycoprotein (transmembrane) Nmb (GPNMB, Accession NM_{—}002510) is another VGAM313 host target gene. GPNMB BINDING SITE is HOST TARGET binding site found in the 3′ untranslated region of mRNA encoded by GPNMB, corresponding to a HOST TARGET binding site such as BINDING SITE I, BINDING SITE II or BINDING SITE III. Table 2 illustrates the complementarity of the nucleotide sequences of GPNMB BINDING SITE, designated SEQ ID:935, to the nucleotide sequence of VGAM313 RNA, herein designated VGAM RNA, also designated SEQ ID:648.

Another function of VGAM313 is therefore inhibition of Glycoprotein (transmembrane) Nmb (GPNMB, Accession NM_{—}002510). Accordingly, utilities of VGAM313 include diagnosis, prevention and treatment of diseases and clinical conditions associated with GPNMB. HSZFP36 (Accession XM_{—}032822) is another VGAM313 host target gene. HSZFP36 BINDING SITE is HOST TARGET binding site found in the 3′ untranslated region of mRNA encoded by HSZFP36, corresponding to a HOST TARGET binding site such as BINDING SITE I, BINDING SITE II or BINDING SITE III. Table 2 illustrates the complementarity of the nucleotide sequences of HSZFP36 BINDING SITE, designated SEQ ID:2659, to the nucleotide sequence of VGAM313 RNA, herein designated VGAM RNA, also designated SEQ ID:648.

Another function of VGAM313 is therefore inhibition of HSZFP36 (Accession XM_{—}032822). Accordingly, utilities of VGAM313 include diagnosis, prevention and treatment of diseases and clinical conditions associated with HSZFP36. IPLA2(GAMMA) (Accession XM_{—}027224) is another VGAM313 host target gene. IPLA2(GAMMA) BINDING SITE is HOST TARGET binding site found in the 3′ untranslated region of mRNA encoded by IPLA2(GAMMA), corresponding to a HOST TARGET binding site such as BINDING SITE I, BINDING SITE II or BINDING SITE III. Table 2 illustrates the complementarity of the nucleotide sequences of IPLA2(GAMMA) BINDING SITE, designated SEQ ID:2577, to the nucleotide sequence of VGAM313 RNA, herein designated VGAM RNA, also designated SEQ ID:648.

Another function of VGAM313 is therefore inhibition of IPLA2(GAMMA) (Accession XM_{—}027224). Accordingly, utilities of VGAM313 include diagnosis, prevention and treatment of diseases and clinical conditions associated with IPLA2(GAMMA). KIAA0648 (Accession XM_{—}094043) is another VGAM313 host target gene. KIAA0648 BINDING SITE is HOST TARGET binding site found in the 3′ untranslated region of mRNA encoded by KIAA0648, corresponding to a HOST TARGET binding site such as BINDING SITE I, BINDING SITE II or BINDING SITE III. Table 2 illustrates the complementarity of the nucleotide sequences of KIAA0648 BINDING SITE, designated SEQ ID:3258, to the nucleotide sequence of VGAM313 RNA, herein designated VGAM RNA, also designated SEQ ID:648.

Another function of VGAM313 is therefore inhibition of KIAA0648 (Accession XM_{—}094043). Accordingly, utilities of VGAM313 include diagnosis, prevention and treatment of diseases and clinical conditions associated with KIAA0648. KIAA1239 (Accession XM_{—}049078) is another VGAM313 host target gene. KIAA1239 BINDING SITE is HOST TARGET binding site found in the 3′ untranslated region of mRNA encoded by KIAA1239, corresponding to a HOST TARGET binding site such as BINDING SITE I, BINDING SITE II or BINDING SITE III. Table 2 illustrates the complementarity of the nucleotide sequences of KIAA1239 BINDING SITE, designated SEQ ID:2915, to the nucleotide sequence of VGAM313 RNA, herein designated VGAM RNA, also designated SEQ ID:648.

Another function of VGAM313 is therefore inhibition of KIAA1239 (Accession XM_{—}049078). Accordingly, utilities of VGAM313 include diagnosis, prevention and treatment of diseases and clinical conditions associated with KIAA1239. KIAA1577 (Accession XM_{—}035299) is another VGAM313 host target gene. KIAA1577 BINDING SITE is HOST TARGET binding site found in the 5′ untranslated region of mRNA encoded by KIAA1577, corresponding to a HOST TARGET binding site such as BINDING SITE I, BINDING SITE II or BINDING SITE III. Table 2 illustrates the complementarity of the nucleotide sequences of KIAA1577 BINDING SITE, designated SEQ ID:2691, to the nucleotide sequence of VGAM313 RNA, herein designated VGAM RNA, also designated SEQ ID:648.

Another function of VGAM313 is therefore inhibition of KIAA1577 (Accession XM_{—}035299). Accordingly, utilities of VGAM313 include diagnosis, prevention and treatment of diseases and clinical conditions associated with KIAA1577. KIAA1821 (Accession XM_{—}050101) is another VGAM313 host target gene. KIAA1821 BINDING SITE is HOST TARGET binding site found in the 3′ untranslated region of mRNA encoded by KIAA1821, corresponding to a HOST TARGET binding site such as BINDING SITE I, BINDING SITE II or BINDING SITE III. Table 2 illustrates the complementarity of the nucleotide sequences of KIAA1821 BINDING SITE, designated SEQ ID:2926, to the nucleotide sequence of VGAM313 RNA, herein designated VGAM RNA, also designated SEQ ID:648.

Another function of VGAM313 is therefore inhibition of KIAA1821 (Accession XM_{—}050101). Accordingly, utilities of VGAM313 include diagnosis, prevention and treatment of diseases and clinical conditions associated with KIAA1821. KIAA1853 (Accession XM_{—}045184) is another VGAM313 host target gene. KIAA1853 BINDING SITE is HOST TARGET binding site found in the 5′ untranslated region of mRNA encoded by KIAA1853, corresponding to a HOST TARGET binding site such as BINDING SITE I, BINDING SITE II or BINDING SITE III. Table 2 illustrates the complementarity of the nucleotide sequences of KIAA1853 BINDING SITE, designated SEQ ID:2857, to the nucleotide sequence of VGAM313 RNA, herein designated VGAM RNA, also designated SEQ ID:648.

Another function of VGAM313 is therefore inhibition of KIAA1853 (Accession XM_{—}045184). Accordingly, utilities of VGAM313 include diagnosis, prevention and treatment of diseases and clinical conditions associated with KIAA1853. MGC3169 (Accession NM_{—}024074) is another VGAM313 host target gene. MGC3169 BINDING SITE is HOST TARGET binding site found in the 3′ untranslated region of mRNA encoded by MGC3169, corresponding to a HOST TARGET binding site such as BINDING SITE I, BINDING SITE II or BINDING SITE III. Table 2 illustrates the complementarity of the nucleotide sequences of MGC3169 BINDING SITE, designated SEQ ID:2051, to the nucleotide sequence of VGAM313 RNA, herein designated VGAM RNA, also designated SEQ ID:648.

Another function of VGAM313 is therefore inhibition of MGC3169 (Accession NM_{—}024074). Accordingly, utilities of VGAM313 include diagnosis, prevention and treatment of diseases and clinical conditions associated with MGC3169. MST4 (Accession NM_{—}016542) is another VGAM313 host target gene. MST4 BINDING SITE is HOST TARGET binding site found in the 3′ untranslated region of mRNA encoded by MST4, corresponding to a HOST TARGET binding site such as BINDING SITE I, BINDING SITE II or BINDING SITE III. Table 2 illustrates the complementarity of the nucleotide sequences of MST4 BINDING SITE, designated SEQ ID: 1696, to the nucleotide sequence of VGAM313 RNA, herein designated VGAM RNA, also designated SEQ ID:648.

Another function of VGAM313 is therefore inhibition of MST4 (Accession NM_{—}016542). Accordingly, utilities of VGAM313 include diagnosis, prevention and treatment of diseases and clinical conditions associated with MST4. Phosphodiesterase 10A (PDE10A, Accession NM_{—}006661) is another VGAM313 host target gene. PDE10A BINDING SITE is HOST TARGET binding site found in the 3′ untranslated region of mRNA encoded by PDE10A, corresponding to a HOST TARGET binding site such as BINDING SITE I, BINDING SITE II or BINDING SITE III. Table 2 illustrates the complementarity of the nucleotide sequences of PDE10A BINDING SITE, designated SEQ ID:1321, to the nucleotide sequence of VGAM313 RNA, herein designated VGAM RNA, also designated SEQ ID:648.

Another function of VGAM313 is therefore inhibition of Phosphodiesterase 10A (PDE10A, Accession NM_{—}006661). Accordingly, utilities of VGAM313 include diagnosis, prevention and treatment of diseases and clinical conditions associated with PDE10A. SKD3 (Accession NM_{—}030813) is another VGAM313 host target gene. SKD3 BINDING SITE is HOST TARGET binding site found in the 3′ untranslated region of mRNA encoded by SKD3, corresponding to a HOST TARGET binding site such as BINDING SITE I, BINDING SITE II or BINDING SITE III. Table 2 illustrates the complementarity of the nucleotide sequences of SKD3 BINDING SITE, designated SEQ ID:2170, to the nucleotide sequence of VGAM313 RNA, herein designated VGAM RNA, also designated SEQ ID:648.

Another function of VGAM313 is therefore inhibition of SKD3 (Accession NM_{—}030813). Accordingly, utilities of VGAM313 include diagnosis, prevention and treatment of diseases and clinical conditions associated with SKD3. Syntaxin 18 (STX18, Accession NM_{—}016930) is another VGAM313 host target gene. STX18 BINDING SITE is HOST TARGET binding site found in the 3′ untranslated region of mRNA encoded by STX18, corresponding to a HOST TARGET binding site such as BINDING SITE I, BINDING SITE II or BINDING SITE III. Table 2 illustrates the complementarity of the nucleotide sequences of STX18 BINDING SITE, designated SEQ ID:1714, to the nucleotide sequence of VGAM313 RNA, herein designated VGAM RNA, also designated SEQ ID:648.

Another function of VGAM313 is therefore inhibition of Syntaxin 18 (STX18, Accession NM_{—}016930). Accordingly, utilities of VGAM313 include diagnosis, prevention and treatment of diseases and clinical conditions associated with STX18. LOC115811 (Accession NM_{—}138451) is another VGAM313 host target gene. LOC115811 BINDING SITE is HOST TARGET binding site found in the 5′ untranslated region of mRNA encoded by LOC115811, corresponding to a HOST TARGET binding site such as BINDING SITE I, BINDING SITE II or BINDING SITE III. Table 2 illustrates the complementarity of the nucleotide sequences of LOC115811 BINDING SITE, designated SEQ ID:2441, to the nucleotide sequence of VGAM313 RNA, herein designated VGAM RNA, also designated SEQ ID:648.

Another function of VGAM313 is therefore inhibition of LOC115811 (Accession NM_{—}138451). Accordingly, utilities of VGAM313 include diagnosis, prevention and treatment of diseases and clinical conditions associated with LOC115811. LOC131308 (Accession XM_{—}059501) is another VGAM313 host target gene. LOC131308 BINDING SITE is HOST TARGET binding site found in the 3′ untranslated region of mRNA encoded by LOC131308, corresponding to a HOST TARGET binding site such as BINDING SITE I, BINDING SITE II or BINDING SITE III. Table 2 illustrates the complementarity of the nucleotide sequences of LOC131308 BINDING SITE, designated SEQ ID:3013, to the nucleotide sequence of VGAM313 RNA, herein designated VGAM RNA, also designated SEQ ID:648.

Another function of VGAM313 is therefore inhibition of LOC131308 (Accession XM_{—}059501). Accordingly, utilities of VGAM313 include diagnosis, prevention and treatment of diseases and clinical conditions associated with LOC131308. LOC139221 (Accession XM_{—}066558) is another VGAM313 host target gene. LOC139221 BINDING SITE is HOST TARGET binding site found in the 3′ untranslated region of mRNA encoded by LOC139221, corresponding to a HOST TARGET binding site such as BINDING SITE I, BINDING SITE II or BINDING SITE III. Table 2 illustrates the complementarity of the nucleotide sequences of LOC139221 BINDING SITE, designated SEQ ID:3036, to the nucleotide sequence of VGAM313 RNA, herein designated VGAM RNA, also designated SEQ ID:648.

Another function of VGAM313 is therefore inhibition of LOC139221 (Accession XM_{—}066558). Accordingly, utilities of VGAM313 include diagnosis, prevention and treatment of diseases and clinical conditions associated with LOC139221. LOC158490 (Accession XM_{—}088585) is another VGAM313 host target gene. LOC158490 BINDING SITE is HOST TARGET binding site found in the 5′ untranslated region of mRNA encoded by LOC158490, corresponding to a HOST TARGET binding site such as BINDING SITE I, BINDING SITE II or BINDING SITE III. Table 2 illustrates the complementarity of the nucleotide sequences of LOC158490 BINDING SITE, designated SEQ ID:3220, to the nucleotide sequence of VGAM313 RNA, herein designated VGAM RNA, also designated SEQ ID:648.

Another function of VGAM313 is therefore inhibition of LOC158490 (Accession XM_{—}088585). Accordingly, utilities of VGAM313 include diagnosis, prevention and treatment of diseases and clinical conditions associated with LOC158490. LOC221466 (Accession XM_{—}168087) is another VGAM313 host target gene. LOC221466 BINDING SITE is HOST TARGET binding site found in the 5′ untranslated region of mRNA encoded by LOC221466, corresponding to a HOST TARGET binding site such as BINDING SITE I, BINDING SITE II or BINDING SITE III. Table 2 illustrates the complementarity of the nucleotide sequences of LOC221466 BINDING SITE, designated SEQ ID:3618, to the nucleotide sequence of VGAM313 RNA, herein designated VGAM RNA, also designated SEQ ID:648.

Another function of VGAM313 is therefore inhibition of LOC221466 (Accession XM_{—}168087). Accordingly, utilities of VGAM313 include diagnosis, prevention and treatment of diseases and clinical conditions associated with LOC221466. LOC221540 (Accession XM_{—}168133) is another VGAM313 host target gene. LOC221540 BINDING SITE is HOST TARGET binding site found in the 3′ untranslated region of mRNA encoded by LOC221540, corresponding to a HOST TARGET binding site such as BINDING SITE I, BINDING SITE II or BINDING SITE III. Table 2 illustrates the complementarity of the nucleotide sequences of LOC221540 BINDING SITE, designated SEQ ID:3621, to the nucleotide sequence of VGAM313 RNA, herein designated VGAM RNA, also designated SEQ ID:648.

Another function of VGAM313 is therefore inhibition of LOC221540 (Accession XM_{—}168133). Accordingly, utilities of VGAM313 include diagnosis, prevention and treatment of diseases and clinical conditions associated with LOC221540. LOC222233 (Accession XM_{—}168560) is another VGAM313 host target gene. LOC222233 BINDING SITE is HOST TARGET binding site found in the 5′ untranslated region of mRNA encoded by LOC222233, corresponding to a HOST TARGET binding site such as BINDING SITE I, BINDING SITE II or BINDING SITE III. Table 2 illustrates the complementarity of the nucleotide sequences of LOC222233 BINDING SITE, designated SEQ ID:3648, to the nucleotide sequence of VGAM313 RNA, herein designated VGAM RNA, also designated SEQ ID:648.

Another function of VGAM313 is therefore inhibition of LOC222233 (Accession XM_{—}168560). Accordingly, utilities of VGAM313 include diagnosis, prevention and treatment of diseases and clinical conditions associated with LOC222233. LOC255042 (Accession XM_{—}170896) is another VGAM313 host target gene. LOC255042 BINDING SITE is HOST TARGET binding site found in the 3′ untranslated region of mRNA encoded by LOC255042, corresponding to a HOST TARGET binding site such as BINDING SITE I, BINDING SITE II or BINDING SITE III. Table 2 illustrates the complementarity of the nucleotide sequences of LOC255042 BINDING SITE, designated SEQ ID:3671, to the nucleotide sequence of VGAM313 RNA, herein designated VGAM RNA, also designated SEQ ID:648.

Another function of VGAM313 is therefore inhibition of LOC255042 (Accession XM_{—}170896). Accordingly, utilities of VGAM313 include diagnosis, prevention and treatment of diseases and clinical conditions associated with LOC255042. LOC256117 (Accession XM_{—}172828) is another VGAM313 host target gene. LOC256117 BINDING SITE is HOST TARGET binding site found in the 3′ untranslated region of mRNA encoded by LOC256117, corresponding to a HOST TARGET binding site such as BINDING SITE I, BINDING SITE II or BINDING SITE III. Table 2 illustrates the complementarity of the nucleotide sequences of LOC256117 BINDING SITE, designated SEQ ID:3705, to the nucleotide sequence of VGAM313 RNA, herein designated VGAM RNA, also designated SEQ ID:648.

Another function of VGAM313 is therefore inhibition of LOC256117 (Accession XM_{—}172828). Accordingly, utilities of VGAM313 include diagnosis, prevention and treatment of diseases and clinical conditions associated with LOC256117. LOC257545 (Accession XM_{—}175217) is another VGAM313 host target gene. LOC257545 BINDING SITE is HOST TARGET binding site found in the 3′ untranslated region of mRNA encoded by LOC257545, corresponding to a HOST TARGET binding site such as BINDING SITE I, BINDING SITE II or BINDING SITE III. Table 2 illustrates the complementarity of the nucleotide sequences of LOC257545 BINDING SITE, designated SEQ ID:3747, to the nucleotide sequence of VGAM313 RNA, herein designated VGAM RNA, also designated SEQ ID:648.

Another function of VGAM313 is therefore inhibition of LOC257545 (Accession XM_{—}175217). Accordingly, utilities of VGAM313 include diagnosis, prevention and treatment of diseases and clinical conditions associated with LOC257545. LOC257598 (Accession XM_{—}175295) is another VGAM313 host target gene. LOC257598 BINDING SITE is HOST TARGET binding site found in the 3′ untranslated region of mRNA encoded by LOC257598, corresponding to a HOST TARGET binding site such as BINDING SITE I, BINDING SITE II or BINDING SITE III. Table 2 illustrates the complementarity of the nucleotide sequences of LOC257598 BINDING SITE, designated SEQ ID:3750, to the nucleotide sequence of VGAM313 RNA, herein designated VGAM RNA, also designated SEQ ID:648.

Another function of VGAM313 is therefore inhibition of LOC257598 (Accession XM_{—}175295). Accordingly, utilities of VGAM313 include diagnosis, prevention and treatment of diseases and clinical conditions associated with LOC257598. FIG. 1 further provides a conceptual description of a novel bioinformatically detected viral gene of the present invention, referred to here as Viral Genomic Address Messenger 314 (VGAM314) viral gene, which modulates expression of respective host target genes thereof, the function and utility of which host target genes is known in the art.

VGAM314 is a novel bioinformatically detected regulatory, non protein coding, viral micro RNA (miRNA) gene. The method by which VGAM314 was detected is described hereinabove with reference to FIGS. 1-8.

VGAM314 gene, herein designated VGAM GENE, is a viral gene contained in the genome of Vaccinia Virus. VGAM314 host target gene, herein designated VGAM HOST TARGET GENE, is a human gene contained in the human genome.

VGAM314 gene encodes a VGAM314 precursor RNA, herein designated VGAM PRECURSOR RNA. Similar to other miRNA genes, and unlike most ordinary genes, VGAM314 precursor RNA does not encode a protein. A nucleotide sequence identical or highly similar to the nucleotide sequence of VGAM314 precursor RNA is designated SEQ ID:300, and is provided hereinbelow with reference to the sequence listing part. Nucleotide sequence SEQ ID:300 is located at position 10620 relative to the genome of Vaccinia Virus.

VGAM314 precursor RNA folds onto itself, forming VGAM314 folded precursor RNA, herein designated VGAM FOLDED PRECURSOR RNA, which has a two-dimensional ‘hairpin structure’. As is well known in the art, this ‘hairpin structure ’ is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM314 folded precursor RNA into VGAM314 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 86%) nucleotide sequence of VGAM314 RNA is designated SEQ ID:649, and is provided hereinbelow with reference to the sequence listing part.

VGAM314 host target gene, herein designated VGAM HOST TARGET GENE, encodes a corresponding messenger RNA, VGAM314 host target RNA, herein designated VGAM HOST TARGET RNA. VGAM314 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM314 RNA, herein designated VGAM RNA, binds complementarily to one or more host target binding sites located in untranslated regions of VGAM314 host target RNA, herein designated VGAM HOST TARGET RNA. This complementary binding is due to the fact that the nucleotide sequence of VGAM314 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM314 RNA, herein designated VGAM RNA, may have a different number of host target binding sites in untranslated regions of a VGAM314 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM314 RNA, herein designated VGAM RNA, to host target binding sites on VGAM314 host target RNA, herein designated VGAM HOST TARGET RNA, such as BINDING SITE I, BINDING SITE II and BINDING SITE III, inhibits translation of VGAM314 host target RNA into VGAM314 host target protein, herein designated VGAM HOST TARGET PROTEIN. VGAM host target protein is therefore outlined by a broken line.

It is appreciated that VGAM314 host target gene, herein designated VGAM HOST TARGET GENE, in fact represents a plurality of VGAM314 host target genes. The mRNA of each one of this plurality of VGAM314 host target genes comprises one or more host target binding sites, each having a nucleotide sequence which is at least partly complementary to VGAM314 RNA, herein designated VGAM RNA, and which when bound by VGAM314 RNA causes inhibition of translation of respective one or more VGAM314 host target proteins.

It is further appreciated by one skilled in the art that the mode of translational inhibition illustrated by FIG. 1 with specific reference to translational inhibition exerted by VGAM314 gene, herein designated VGAM GENE, on one or more VGAM314 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM314 is inhibition of expression of host target genes, as part of a novel viral mechanism of attacking a host. Accordingly, utilities of VGAM314 include diagnosis, prevention and treatment of viral infection by Vaccinia Virus. Specific functions, and accordingly utilities, of VGAM314 correlate with, and may be deduced from, the identity of the host target genes which VGAM314 binds and inhibits, and the function of these host target genes, as elaborated hereinbelow.

Nucleotide sequences of the VGAM314 precursor RNA, herein designated VGAM PRECURSOR RNA, and of the ‘diced’ VGAM314 RNA, herein designated VGAM RNA, and a schematic representation of the secondary folding of VGAM314 folded precursor RNA, herein designated VGAM FOLDED PRECURSOR RNA, of VGAM314 are further described hereinbelow with reference to Table 1.

Nucleotide sequences of host target binding sites, such as BINDING SITE-I, BINDING SITE-II and BINDING SITE-III of FIG. 1, found on VGAM314 host target RNA, and schematic representation of the complementarity of each of these host target binding sites to VGAM314 RNA, herein designated VGAM RNA, are described hereinbelow with reference to Table 2.

As mentioned hereinabove with reference to FIG. 1, a function of VGAM314 gene, herein designated VGAM is inhibition of expression of VGAM314 target genes. It is appreciated that specific functions, and accordingly utilities, of VGAM314 correlate with, and may be deduced from, the identity of the target genes which VGAM314 binds and inhibits, and the function of these target genes, as elaborated hereinbelow.

Chorea Acanthocytosis (CHAC, Accession NM_{—}015186) is a VGAM314 host target gene. CHAC BINDING SITE is HOST TARGET binding site found in the 3′ untranslated region of mRNA encoded by CHAC, corresponding to a HOST TARGET binding site such as BINDING SITE I, BINDING SITE II or BINDING SITE III. Table 2 illustrates the complementarity of the nucleotide sequences of CHAC BINDING SITE, designated SEQ ID: 1612, to the nucleotide sequence of VGAM314 RNA, herein designated VGAM RNA, also designated SEQ ID:649.

A function of VGAM314 is therefore inhibition of Chorea Acanthocytosis (CHAC, Accession NM_{—}015186), a gene which may regulate the cycling of proteins. Accordingly, utilities of VGAM314 include diagnosis, prevention and treatment of diseases and clinical conditions associated with CHAC. The function of CHAC and its association with various diseases and clinical conditions, has been established by previous studies, as described hereinabove with reference to VGAM68. Gamma-glutamyl Carboxylase (GGCX, Accession NM_{—}000821) is another VGAM314 host target gene. GGCX BINDING SITE is HOST TARGET binding site found in the 3′ untranslated region of mRNA encoded by GGCX, corresponding to a HOST TARGET binding site such as BINDING SITE I, BINDING SITE II or BINDING SITE III. Table 2 illustrates the complementarity of the nucleotide sequences of GGCX BINDING SITE, designated SEQ ID:778, to the nucleotide sequence of VGAM314 RNA, herein designated VGAM RNA, also designated SEQ ID:649.

Another function of VGAM314 is therefore inhibition of Gamma-glutamyl Carboxylase (GGCX, Accession NM_{—}000821). Accordingly, utilities of VGAM314 include diagnosis, prevention and treatment of diseases and clinical conditions associated with GGCX. Inositol 1,4,5-trisphosphate 3-kinase B (ITPKB, Accession NM_{—}002221) is another VGAM314 host target gene. ITPKB BINDING SITE is HOST TARGET binding site found in the 3′ untranslated region of mRNA encoded by ITPKB, corresponding to a HOST TARGET binding site such as BINDING SITE I, BINDING SITE II or BINDING SITE III. Table 2 illustrates the complementarity of the nucleotide sequences of ITPKB BINDING SITE, designated SEQ ID:911, to the nucleotide sequence of VGAM314 RNA, herein designated VGAM RNA, also designated SEQ ID:649.

Another function of VGAM314 is therefore inhibition of Inositol 1,4,5-trisphosphate 3-kinase B (ITPKB, Accession NM_{—}002221), a gene which is a type B inositol 1,4,5-triphosphate 3 kinase. Accordingly, utilities of VGAM314 include diagnosis, prevention and treatment of diseases and clinical conditions associated with ITPKB. The function of ITPKB and its association with various diseases and clinical conditions, has been established by previous studies, as described hereinabove with reference to VGAM68. Potassium Voltage-gated Channel, Shaker-related Sub family, Beta Member 1 (KCNAB1, Accession XM_{—}027634) is another VGAM314 host target gene. KCNAB1 BINDING SITE is HOST TARGET binding site found in the 3′ untranslated region of mRNA encoded by KCNAB1, corresponding to a HOST TARGET binding site such as BINDING SITE I, BINDING SITE II or BINDING SITE III. Table 2 illustrates the complementarity of the nucleotide sequences of KCNAB1 BINDING SITE, designated SEQ ID:2584, to the nucleotide sequence of VGAM314 RNA, herein designated VGAM RNA, also designated SEQ ID:649.

Another function of VGAM314 is therefore inhibition of Potassium Voltage-gated Channel, Shaker-related Sub family, Beta Member 1 (KCNAB1, Accession XM_{—}027634), a gene which is the regulatory beta subunit for a shaker-related voltage-gated potassium channel. Accordingly, utilities of VGAM314 include diagnosis, prevention and treatment of diseases and clinical conditions associated with KCNAB1. The function of KCNAB1 and its association with various diseases and clinical conditions, has been established by previous studies, as described hereinabove with reference to VGAM68. Killer Cell Lectin-like Receptor Sub family C, Member 4 (KLRC4, Accession NM_{—}013431) is another VGAM314 host target gene. KLRC4 BINDING SITE is HOST TARGET binding site found in the 5′ untranslated region of mRNA encoded by KLRC4, corresponding to a HOST TARGET binding site such as BINDING SITE I, BINDING SITE II or BINDING SITE III. Table 2 illustrates the complementarity of the nucleotide sequences of KLRC4 BINDING SITE, designated SEQ ID:1451, to the nucleotide sequence of VGAM314 RNA, herein designated VGAM RNA, also designated SEQ ID:649.

Another function of VGAM314 is therefore inhibition of Killer Cell Lectin-like Receptor Sub family C, Member 4 (KLRC4, Accession NM_{—}013431), a gene which is a receptor for the recognition of mhc class i hla-e molecules by nk cells and some cytotoxic t-cells. Accordingly, utilities of VGAM314 include diagnosis, prevention and treatment of diseases and clinical conditions associated with KLRC4. The function of KLRC4 and its association with various diseases and clinical conditions, has been established by previous studies, as described hereinabove with reference to VGAM68. Myeloid Leukemia Factor 2 (MLF2, Accession NM_{—}005439) is another VGAM314 host target gene. MLF2 BINDING SITE is HOST TARGET binding site found in the 3′ untranslated region of mRNA encoded by MLF2, corresponding to a HOST TARGET binding site such as BINDING SITE I, BINDING SITE II or BINDING SITE III. Table 2 illustrates the complementarity of the nucleotide sequences of MLF2 BINDING SITE, designated SEQ ID:1212, to the nucleotide sequence of VGAM314 RNA, herein designated VGAM RNA, also designated SEQ ID:649.

Another function of VGAM314 is therefore inhibition of Myeloid Leukemia Factor 2 (MLF2, Accession NM_{—}005439). Accordingly, utilities of VGAM314 include diagnosis, prevention and treatment of diseases and clinical conditions associated with MLF2. Nuclear Receptor Sub family 1, Group 1, Member 2 (NR1I2, Accession NM_{—}022002) is another VGAM314 host target gene. NR1I2 BINDING SITE1 and NR1I2 BINDING SITE2 are HOST TARGET binding sites found in untranslated regions of mRNA encoded by NR1I2, corresponding to HOST TARGET binding sites such as BINDING SITE I, BINDING SITE II or BINDING SITE III. Table 2 illustrates the complementarity of the nucleotide sequences of NR1I2 BINDING SITE1 and NR1I2 BINDING SITE2, designated SEQ ID: 1971 and SEQ ID: 1067 respectively, to the nucleotide sequence of VGAM314 RNA, herein designated VGAM RNA, also designated SEQ ID:649.

Another function of VGAM314 is therefore inhibition of Nuclear Receptor Sub family 1, Group I, Member 2 (NR1I2, Accession NM_{—}022002), a gene which binds to a response element in the cyp3a4 gene promoter and activates its expression in response to a wide variety of endobiotics and xenobiotics. Accordingly, utilities of VGAM314 include diagnosis, prevention and treatment of diseases and clinical conditions associated with NR1I2. The function of NR1I2 and its association with various diseases and clinical conditions, has been established by previous studies, as described hereinabove with reference to VGAM68. FHX (Accession NM_{—}018416) is another VGAM314 host target gene. FHX BINDING SITE is HOST TARGET binding site found in the 3′ untranslated region of mRNA encoded by FHX, corresponding to a HOST TARGET binding site such as BINDING SITE I, BINDING SITE II or BINDING SITE III. Table 2 illustrates the complementarity of the nucleotide sequences of FHX BINDING SITE, designated SEQ ID:1825, to the nucleotide sequence of VGAM314 RNA, herein designated VGAM RNA, also designated SEQ ID:649.

Another function of VGAM314 is therefore inhibition of FHX (Accession NM_{—}018416). Accordingly, utilities of VGAM314 include diagnosis, prevention and treatment of diseases and clinical conditions associated with FHX. FLJ10352 (Accession NM_{—}032142) is another VGAM314 host target gene. FLJ10352 BINDING SITE is HOST TARGET binding site found in the 3′ untranslated region of mRNA encoded by FLJ10352, corresponding to a HOST TARGET binding site such as BINDING SITE I, BINDING SITE II or BINDING SITE III. Table 2 illustrates the complementarity of the nucleotide sequences of FLJ10352 BINDING SITE, designated SEQ ID:2233, to the nucleotide sequence of VGAM314 RNA, herein designated VGAM RNA, also designated SEQ ID:649.

Another function of VGAM314 is therefore inhibition of FLJ10352 (Accession NM_{—}032142). Accordingly, utilities of VGAM314 include diagnosis, prevention and treatment of diseases and clinical conditions associated with FLJ10352. FLJ22127 (Accession NM_{—}022775) is another VGAM314 host target gene. FLJ22127 BINDING SITE is HOST TARGET binding site found in the 3′ untranslated region of mRNA encoded by FLJ22127, corresponding to a HOST TARGET binding site such as BINDING SITE I, BINDING SITE II or BINDING SITE III. Table 2 illustrates the complementarity of the nucleotide sequences of FLJ22127 BINDING SITE, designated SEQ ID:2008, to the nucleotide sequence of VGAM314 RNA, herein designated VGAM RNA, also designated SEQ ID:649.

Another function of VGAM314 is therefore inhibition of FLJ22127 (Accession NM_{—}022775). Accordingly, utilities of VGAM314 include diagnosis, prevention and treatment of diseases and clinical conditions associated with FLJ22127. Ganglioside-induced Differentiation-associated Protein 1-like 1 (GDAP1L1, Accession NM_{—}024034) is another VGAM314 host target gene. GDAP1L1 BINDING SITE is HOST TARGET binding site found in the 3′ untranslated region of mRNA encoded by GDAP1L1, corresponding to a HOST TARGET binding site such as BINDING SITE I, BINDING SITE II or BINDING SITE III. Table 2 illustrates the complementarity of the nucleotide sequences of GDAP1L1 BINDING SITE, designated SEQ ID:2043, to the nucleotide sequence of VGAM314 RNA, herein designated VGAM RNA, also designated SEQ ID:649.

Another function of VGAM314 is therefore inhibition of Ganglioside-induced Differentiation-associated Protein 1-like 1 (GDAP1L1, Accession NM_{—}024034). Accordingly, utilities of VGAM314 include diagnosis, prevention and treatment of diseases and clinical conditions associated with GDAP1L1. H-L(3)MBT (Accession NM_{—}032107) is another VGAM314 host target gene. H-L(3)MBT BINDING SITE is HOST TARGET binding site found in the 3′ untranslated region of mRNA encoded by H-L(3)MBT, corresponding to a HOST TARGET binding site such as BINDING SITE I, BINDING SITE II or BINDING SITE III. Table 2 illustrates the complementarity of the nucleotide sequences of H-L(3)MBT BINDING SITE, designated SEQ ID:2230, to the nucleotide sequence of VGAM314 RNA, herein designated VGAM RNA, also designated SEQ ID:649.

Another function of VGAM314 is therefore inhibition of HL(3)MBT (Accession NM_{—}032107). Accordingly, utilities of VGAM314 include diagnosis, prevention and treatment of diseases and clinical conditions associated with HL(3)MBT. KIAA1340 (Accession XM_{—}044836) is another VGAM314 host target gene. KIAA1340 BINDING SITE is HOST TARGET binding site found in the 3′ untranslated region of mRNA encoded by KIAA1340, corresponding to a HOST TARGET binding site such as BINDING SITE I, BINDING SITE II or BINDING SITE III. Table 2 illustrates the complementarity of the nucleotide sequences of KIAA1340 BINDING SITE, designated SEQ ID:2846, to the nucleotide sequence of VGAM314 RNA, herein designated VGAM RNA, also designated SEQ ID:649.

Another function of VGAM314 is therefore inhibition of KIAA1340 (Accession XM_{—}044836). Accordingly, utilities of VGAM314 include diagnosis, prevention and treatment of diseases and clinical conditions associated with KIAA1340. Kinesin-like 7 (KNSL7, Accession NM_{—}020242) is another VGAM314 host target gene. KNSL7 BINDING SITE is HOST TARGET binding site found in the 3′ untranslated region of mRNA encoded by KNSL7, corresponding to a HOST TARGET binding site such as BINDING SITE I, BINDING SITE II or BINDING SITE III. Table 2 illustrates the complementarity of the nucleotide sequences of KNSL7 BINDING SITE, designated SEQ ID:1905, to the nucleotide sequence of VGAM314 RNA, herein designated VGAM RNA, also designated SEQ ID:649.

Another function of VGAM314 is therefore inhibition of Kinesin-like 7 (KNSL7, Accession NM_{—}020242). Accordingly, utilities of VGAM314 include diagnosis, prevention and treatment of diseases and clinical conditions associated with KNSL7. P5-1 (Accession NM_{—}006674) is another VGAM314 host target gene. P5-1 BINDING SITE is HOST TARGET binding site found in the 3′ untranslated region of mRNA encoded by P5-1, corresponding to a HOST TARGET binding site such as BINDING SITE I, BINDING SITE II or BINDING SITE III. Table 2 illustrates the complementarity of the nucleotide sequences of P5-1 BINDING SITE, designated SEQ ID:1322, to the nucleotide sequence of VGAM314 RNA, herein designated VGAM RNA, also designated SEQ ID:649.

Another function of VGAM314 is therefore inhibition of P5-1 (Accession NM_{—}006674). Accordingly, utilities of VGAM314 include diagnosis, prevention and treatment of diseases and clinical conditions associated with P5-1. PP1044 (Accession NM_{—}021730) is another VGAM314 host target gene. PP1044 BINDING SITE is HOST TARGET binding site found in the 5′ untranslated region of mRNA encoded by PP1044, corresponding to a HOST TARGET binding site such as BINDING SITE I, BINDING SITE II or BINDING SITE III. Table 2 illustrates the complementarity of the nucleotide sequences of PP1044 BINDING SITE, designated SEQ ID: 1956, to the nucleotide sequence of VGAM314 RNA, herein designated VGAM RNA, also designated SEQ ID:649.

Another function of VGAM314 is therefore inhibition of PP1044 (Accession NM_{—}021730). Accordingly, utilities of VGAM314 include diagnosis, prevention and treatment of diseases and clinical conditions associated with PP1044. Signal-regulatory Protein Beta 1 (SIRPB1, Accession NM_{—}006065) is another VGAM314 host target gene. SIRPB1 BINDING SITE is HOST TARGET binding site found in the 3′ untranslated region of mRNA encoded by SIRPB1, corresponding to a HOST TARGET binding site such as BINDING SITE I, BINDING SITE II or BINDING SITE III. Table 2 illustrates the complementarity of the nucleotide sequences of SIRPB1 BINDING SITE, designated SEQ ID:1272, to the nucleotide sequence of VGAM314 RNA, herein designated VGAM RNA, also designated SEQ ID:649.

Another function of VGAM314 is therefore inhibition of Signal-regulatory Protein Beta 1 (SIRPB1, Accession NM_{—}006065). Accordingly, utilities of VGAM314 include diagnosis, prevention and treatment of diseases and clinical conditions associated with SIRPB1. LOC148811 (Accession XM_{—}086326) is another VGAM314 host target gene. LOC148811 BINDING SITE is HOST TARGET binding site found in the 3′ untranslated region of mRNA encoded by LOC148811, corresponding to a HOST TARGET binding site such as BINDING SITE I, BINDING SITE II or BINDING SITE III. Table 2 illustrates the complementarity of the nucleotide sequences of LOC148811 BINDING SITE, designated SEQ ID:3126, to the nucleotide sequence of VGAM314 RNA, herein designated VGAM RNA, also designated SEQ ID:649.

Another function of VGAM314 is therefore inhibition of LOC148811 (Accession XM_{—}086326). Accordingly, utilities of VGAM314 include diagnosis, prevention and treatment of diseases and clinical conditions associated with LOC148811. LOC149711 (Accession XM_{—}097720) is another VGAM314 host target gene. LOC149711 BINDING SITE is HOST TARGET binding site found in the 3′ untranslated region of mRNA encoded by LOC149711, corresponding to a HOST TARGET binding site such as BINDING SITE I, BINDING SITE II or BINDING SITE III. Table 2 illustrates the complementarity of the nucleotide sequences of LOC149711 BINDING SITE, designated SEQ ID:3315, to the nucleotide sequence of VGAM314 RNA, herein designated VGAM RNA, also designated SEQ ID:649.

Another function of VGAM314 is therefore inhibition of LOC149711 (Accession XM_{—}097720). Accordingly, utilities of VGAM314 include diagnosis, prevention and treatment of diseases and clinical conditions associated with LOC149711. LOC150630 (Accession XM_{—}097931) is another VGAM314 host target gene. LOC150630 BINDING SITE is HOST TARGET binding site found in the 5′ untranslated region of mRNA encoded by LOC150630, corresponding to a HOST TARGET binding site such as BINDING SITE I, BINDING SITE II or BINDING SITE III. Table 2 illustrates the complementarity of the nucleotide sequences of LOC150630 BINDING SITE, designated SEQ ID:3332, to the nucleotide sequence of VGAM314 RNA, herein designated VGAM RNA, also designated SEQ ID:649.

Another function of VGAM314 is therefore inhibition of LOC150630 (Accession XM_{—}097931). Accordingly, utilities of VGAM314 include diagnosis, prevention and treatment of diseases and clinical conditions associated with LOC150630. LOC150819 (Accession XM_{—}097954) is another VGAM314 host target gene. LOC150819 BINDING SITE is HOST TARGET binding site found in the 5′ untranslated region of mRNA encoded by LOC150819, corresponding to a HOST TARGET binding site such as BINDING SITE I, BINDING SITE II or BINDING SITE III. Table 2 illustrates the complementarity of the nucleotide sequences of LOC150819 BINDING SITE, designated SEQ ID:3333, to the nucleotide sequence of VGAM314 RNA, herein designated VGAM RNA, also designated SEQ ID:649.

Another function of VGAM314 is therefore inhibition of LOC150819 (Accession XM_{—}097954). Accordingly, utilities of VGAM314 include diagnosis, prevention and treatment of diseases and clinical conditions associated with LOC150819. LOC154877 (Accession XM_{—}098626) is another VGAM314 host target gene. LOC154877 BINDING SITE is HOST TARGET binding site found in the 3′ untranslated region of mRNA encoded by LOC154877, corresponding to a HOST TARGET binding site such as BINDING SITE I, BINDING SITE II or BINDING SITE III. Table 2 illustrates the complementarity of the nucleotide sequences of LOC154877 BINDING SITE, designated SEQ ID:3373, to the nucleotide sequence of VGAM314 RNA, herein designated VGAM RNA, also designated SEQ ID:649.

Another function of VGAM314 is therefore inhibition of LOC154877 (Accession XM_{—}098626). Accordingly, utilities of VGAM314 include diagnosis, prevention and treatment of diseases and clinical conditions associated with LOC154877. LOC158327 (Accession XM_{—}088548) is another VGAM314 host target gene. LOC158327 BINDING SITE is HOST TARGET binding site found in the 3′ untranslated region of mRNA encoded by LOC158327, corresponding to a HOST TARGET binding site such as BINDING SITE I, BINDING SITE II or BINDING SITE III. Table 2 illustrates the complementarity of the nucleotide sequences of LOC158327 BINDING SITE, designated SEQ ID:3216, to the nucleotide sequence of VGAM314 RNA, herein designated VGAM RNA, also designated SEQ ID:649.

Another function of VGAM314 is therefore inhibition of LOC158327 (Accession XM_{—}088548). Accordingly, utilities of VGAM314 include diagnosis, prevention and treatment of diseases and clinical conditions associated with LOC158327. LOC196540 (Accession XM_{—}116933) is another VGAM314 host target gene. LOC196540 BINDING SITE is HOST TARGET binding site found in the 3′ untranslated region of mRNA encoded by LOC196540, corresponding to a HOST TARGET binding site such as BINDING SITE I, BINDING SITE II or BINDING SITE III. Table 2 illustrates the complementarity of the nucleotide sequences of LOC196540 BINDING SITE, designated SEQ ID:3467, to the nucleotide sequence of VGAM314 RNA, herein designated VGAM RNA, also designated SEQ ID:649.

Another function of VGAM314 is therefore inhibition of LOC196540 (Accession XM_{—}116933). Accordingly, utilities of VGAM314 include diagnosis, prevention and treatment of diseases and clinical conditions associated with LOC196540. LOC220394 (Accession XM_{—}166936) is another VGAM314 host target gene. LOC220394 BINDING SITE is HOST TARGET binding site found in the 5′ untranslated region of mRNA encoded by LOC220394, corresponding to a HOST TARGET binding site such as BINDING SITE I, BINDING SITE II or BINDING SITE III. Table 2 illustrates the complementarity of the nucleotide sequences of LOC220394 BINDING SITE, designated SEQ ID:3581, to the nucleotide sequence of VGAM314 RNA, herein designated VGAM RNA, also designated SEQ ID:649.

Another function of VGAM314 is therefore inhibition of LOC220394 (Accession XM_{—}166936). Accordingly, utilities of VGAM314 include diagnosis, prevention and treatment of diseases and clinical conditions associated with LOC220394. LOC254428 (Accession XM_{—}170932) is another VGAM314 host target gene. LOC254428 BINDING SITE is HOST TARGET binding site found in the 3′ untranslated region of mRNA encoded by LOC254428, corresponding to a HOST TARGET binding site such as BINDING SITE I, BINDING SITE II or BINDING SITE III. Table 2 illustrates the complementarity of the nucleotide sequences of LOC254428 BINDING SITE, designated SEQ ID:3677, to the nucleotide sequence of VGAM314 RNA, herein designated VGAM RNA, also designated SEQ ID:649.

Another function of VGAM314 is therefore inhibition of LOC254428 (Accession XM_{—}170932). Accordingly, utilities of VGAM314 include diagnosis, prevention and treatment of diseases and clinical conditions associated with LOC254428. LOC92405 (Accession XM_{—}044914) is another VGAM314 host target gene. LOC92405 BINDING SITE is HOST TARGET binding site found in the 3′ untranslated region of mRNA encoded by LOC92405, corresponding to a HOST TARGET binding site such as BINDING SITE I, BINDING SITE II or BINDING SITE III. Table 2 illustrates the complementarity of the nucleotide sequences of LOC92405 BINDING SITE, designated SEQ ID:2848, to the nucleotide sequence of VGAM314 RNA, herein designated VGAM RNA, also designated SEQ ID:649.

Another function of VGAM314 is therefore inhibition of LOC92405 (Accession XM_{—}044914). Accordingly, utilities of VGAM314 include diagnosis, prevention and treatment of diseases and clinical conditions associated with LOC92405. FIG. 1 further provides a conceptual description of a novel bioinformatically detected viral gene of the present invention, referred to here as Viral Genomic Address Messenger 315 (VGAM315) viral gene, which modulates expression of respective host target genes thereof, the function and utility of which host target genes is known in the art.

VGAM315 is a novel bioinformatically detected regulatory, non protein coding, viral micro RNA (miRNA) gene. The method by which VGAM315 was detected is described hereinabove with reference to FIGS. 1-8.

VGAM315 gene, herein designated VGAM GENE, is a viral gene contained in the genome of Vaccinia Virus. VGAM315 host target gene, herein designated VGAM HOST TARGET GENE, is a human gene contained in the human genome.

VGAM315 gene encodes a VGAM315 precursor RNA, herein designated VGAM PRECURSOR RNA. Similar to other miRNA genes, and unlike most ordinary genes, VGAM315 precursor RNA does not encode a protein. A nucleotide sequence identical or highly similar to the nucleotide sequence of VGAM315 precursor RNA is designated SEQ ID:301, and is provided hereinbelow with reference to the sequence listing part. Nucleotide sequence SEQ ID:301 is located at position 179807 relative to the genome of Vaccinia Virus.

VGAM315 precursor RNA folds onto itself, forming VGAM315 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM315 folded precursor RNA into VGAM315 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM315 RNA is designated SEQ ID:650, and is provided hereinbelow with reference to the sequence listing part.

VGAM315 host target gene, herein designated VGAM HOST TARGET GENE, encodes a corresponding messenger RNA, VGAM315 host target RNA, herein designated VGAM HOST TARGET RNA. VGAM315 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM315 RNA, herein designated VGAM RNA, binds complementarily to one or more host target binding sites located in untranslated regions of VGAM315 host target RNA, herein designated VGAM HOST TARGET RNA. This complementary binding is due to the fact that the nucleotide sequence of VGAM315 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM315 RNA, herein designated VGAM RNA, may have a different number of host target binding sites in untranslated regions of a VGAM315 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM315 RNA, herein designated VGAM RNA, to host target binding sites on VGAM315 host target RNA, herein designated VGAM HOST TARGET RNA, such as BINDING SITE I, BINDING SITE II and BINDING SITE III, inhibits translation of VGAM315 host target RNA into VGAM315 host target protein, herein designated VGAM HOST TARGET PROTEIN. VGAM host target protein is therefore outlined by a broken line.

It is appreciated that VGAM315 host target gene, herein designated VGAM HOST TARGET GENE, in fact represents a plurality of VGAM315 host target genes. The mRNA of each one of this plurality of VGAM315 host target genes comprises one or more host target binding sites, each having a nucleotide sequence which is at least partly complementary to VGAM315 RNA, herein designated VGAM RNA, and which when bound by VGAM315 RNA causes inhibition of translation of respective one or more VGAM315 host target proteins.

It is further appreciated by one skilled in the art that the mode of translational inhibition illustrated by FIG. 1 with specific reference to translational inhibition exerted by VGAM315 gene, herein designated VGAM GENE, on one or more VGAM315 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM315 is inhibition of expression of host target genes, as part of a novel viral mechanism of attacking a host. Accordingly, utilities of VGAM315 include diagnosis, prevention and treatment of viral infection by Vaccinia Virus. Specific functions, and accordingly utilities, of VGAM315 correlate with, and may be deduced from, the identity of the host target genes which VGAM315 binds and inhibits, and the function of these host target genes, as elaborated hereinbelow.

Nucleotide sequences of the VGAM315 precursor RNA, herein designated VGAM PRECURSOR RNA, and of the ‘diced’ VGAM315 RNA, herein designated VGAM RNA, and a schematic representation of the secondary folding of VGAM315 folded precursor RNA, herein designated VGAM FOLDED PRECURSOR RNA, of VGAM315 are further described hereinbelow with reference to Table 1.

Nucleotide sequences of host target binding sites, such as BINDING SITE-I, BINDING SITE-II and BINDING SITE-III of FIG. 1, found on VGAM315 host target RNA, and schematic representation of the complementarity of each of these host target binding sites to VGAM315 RNA, herein designated VGAM RNA, are described hereinbelow with reference to Table 2.

As mentioned hereinabove with reference to FIG. 1, a function of VGAM315 gene, herein designated VGAM is inhibition of expression of VGAM315 target genes. It is appreciated that specific functions, and accordingly utilities, of VGAM315 correlate with, and may be deduced from, the identity of the target genes which VGAM315 binds and inhibits, and the function of these target genes, as elaborated hereinbelow.

Phosphatidylinositol Glycan, Class H (PIGH, Accession NM_{—}004569) is a VGAM315 host target gene. PIGH BINDING SITE is HOST TARGET binding site found in the 3′ untranslated region of mRNA encoded by PIGH, corresponding to a HOST TARGET binding site such as BINDING SITE I, BINDING SITE II or BINDING SITE III. Table 2 illustrates the complementarity of the nucleotide sequences of PIGH BINDING SITE, designated SEQ ID:1126, to the nucleotide sequence of VGAM315 RNA, herein designated VGAM RNA, also designated SEQ ID:650.

A function of VGAM315 is therefore inhibition of Phosphatidylinositol Glycan, Class H (PIGH, Accession NM_{—}004569), a gene which catalyzes transfer of GlcNAc to PI. Accordingly, utilities of VGAM315 include diagnosis, prevention and treatment of diseases and clinical conditions associated with PIGH. The function of PIGH has been established by previous studies. The PIGH gene encodes an enzyme involved in the biosynthesis of glycosylphosphatidylinositol anchor; see PIGA (OMIM Ref. No. 311770) and PIGF (OMIM Ref. No. 600153) for other components of the system. Kamitani et al. (1993) isolated cDNA for a human gene that repaired the defect in a complementation class H mutant cell line. They determined that PIGH encodes a predicted protein of 188 amino acids. Ware et al. (1994) demonstrated that the mouse Pigh gene is located on chromosome 12 in a region of homology of synteny with 14q11-q24. Watanabe et al. (1996) demonstrated that the PIGA and PIGH proteins form a protein complex and are subunits of the GPI GlcNAc transferase of the endoplasmic reticulum (ER). They showed that PIGH is a cytoplasmic ER-associated protein. Using immunoprecipitation experiments, Watanabe et al. (1998) demonstrated that PIGQ (OMIM Ref. No. 605754) associates specifically with PIGA, PIGC (OMIM Ref. No. 601730), and PIGH and that all 4 proteins form a complex that has GPI-GlcNAc transferase (GPI-GnT) activity in vitro

Full details of the abovementioned studies are described in the following publications, the disclosure of which are hereby incorporated by reference:
Watanabe, R.; Inoue, N.; Westfall, B.; Taron, C. H.; Orlean, P.; Takeda, J.; Kinoshita, T.: The first step of glycosylphosphatidylinositol biosynthesis is mediated by a complex of PIG-A, PIG-H, PIG-C and GPI1. EMBO J. 17: 877-885, 1998.; and
Watanabe, R.; Kinoshita, T.; Masaki, R.; Yamamoto, A.; Takeda, J.; Inoue, N.: PIG-A and PIG-H, which participate in glycosylphosphatidylinositol anchor biosynthesis, form a protein comp.

Further studies establishing the function and utilities of PIGH are found in John Hopkins OMIM database record ID 600154, and in sited publications numbered 32 and 1853-1855 listed in the bibliography section hereinbelow, which are also hereby incorporated by reference. PRSC (Accession NM_{—}006587) is another VGAM315 host target gene. PRSC BINDING SITE is HOST TARGET binding site found in the 3′ untranslated region of mRNA encoded by PRSC, corresponding to a HOST TARGET binding site such as BINDING SITE I, BINDING SITE II or BINDING SITE III. Table 2 illustrates the complementarity of the nucleotide sequences of PRSC BINDING SITE, designated SEQ ID:1313, to the nucleotide sequence of VGAM315 RNA, herein designated VGAM RNA, also designated SEQ ID:650.

Another function of VGAM315 is therefore inhibition of PRSC (Accession NM_{—}006587). Accordingly, utilities of VGAM315 include diagnosis, prevention and treatment of diseases and clinical conditions associated with PRSC. Peroxisomal Membrane Protein 4, 24 kDa (PXMP4, Accession NM_{—}007238) is another VGAM315 host target gene. PXMP4 BINDING SITE is HOST TARGET binding site found in the 3′ untranslated region of mRNA encoded by PXMP4, corresponding to a HOST TARGET binding site such as BINDING SITE I, BINDING SITE II or BINDING SITE III. Table 2 illustrates the complementarity of the nucleotide sequences of PXMP4 BINDING SITE, designated SEQ ID:1370, to the nucleotide sequence of VGAM315 RNA, herein designated VGAM RNA, also designated SEQ ID:650.

Another function of VGAM315 is therefore inhibition of Peroxisomal Membrane Protein 4, 24 kDa (PXMP4, Accession NM_{—}007238). Accordingly, utilities of VGAM315 include diagnosis, prevention and treatment of diseases and clinical conditions associated with PXMP4. FIG. 1 further provides a conceptual description of a novel bioinformatically detected viral gene of the present invention, referred to here as Viral Genomic Address Messenger 316 (VGAM316) viral gene, which modulates expression of respective host target genes thereof, the function and utility of which host target genes is known in the art.

VGAM316 is a novel bioinformatically detected regulatory, non protein coding, viral micro RNA (miRNA) gene. The method by which VGAM316 was detected is described hereinabove with reference to FIGS. 1-8.

VGAM316 gene, herein designated VGAM GENE, is a viral gene contained in the genome of Vaccinia Virus. VGAM316 host target gene, herein designated VGAM HOST TARGET GENE, is a human gene contained in the human genome.

VGAM316 gene encodes a VGAM316 precursor RNA, herein designated VGAM PRECURSOR RNA. Similar to other miRNA genes, and unlike most ordinary genes, VGAM316 precursor RNA does not encode a protein. A nucleotide sequence identical or highly similar to the nucleotide sequence of VGAM316 precursor RNA is designated SEQ ID:302, and is provided hereinbelow with reference to the sequence listing part. Nucleotide sequence SEQ ID:302 is located at position 6123 relative to the genome of Vaccinia Virus.

VGAM316 precursor RNA folds onto itself, forming VGAM316 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM316 folded precursor RNA into VGAM316 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM316 RNA is designated SEQ ID:651, and is provided hereinbelow with reference to the sequence listing part.

VGAM316 host target gene, herein designated VGAM HOST TARGET GENE, encodes a corresponding messenger RNA, VGAM316 host target RNA, herein designated VGAM HOST TARGET RNA. VGAM316 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM316 RNA, herein designated VGAM RNA, binds complementarily to one or more host target binding sites located in untranslated regions of VGAM316 host target RNA, herein designated VGAM HOST TARGET RNA. This complementary binding is due to the fact that the nucleotide sequence of VGAM316 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM316 RNA, herein designated VGAM RNA, may have a different number of host target binding sites in untranslated regions of a VGAM316 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM316 RNA, herein designated VGAM RNA, to host target binding sites on VGAM316 host target RNA, herein designated VGAM HOST TARGET RNA, such as BINDING SITE I, BINDING SITE II and BINDING SITE III, inhibits translation of VGAM316 host target RNA into VGAM316 host target protein, herein designated VGAM HOST TARGET PROTEIN. VGAM host target protein is therefore outlined by a broken line.

It is appreciated that VGAM316 host target gene, herein designated VGAM HOST TARGET GENE, in fact represents a plurality of VGAM316 host target genes. The mRNA of each one of this plurality of VGAM316 host target genes comprises one or more host target binding sites, each having a nucleotide sequence which is at least partly complementary to VGAM316 RNA, herein designated VGAM RNA, and which when bound by VGAM316 RNA causes inhibition of translation of respective one or more VGAM316 host target proteins.

It is further appreciated by one skilled in the art that the mode of translational inhibition illustrated by FIG. 1 with specific reference to translational inhibition exerted by VGAM316 gene, herein designated VGAM GENE, on one or more VGAM316 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM316 is inhibition of expression of host target genes, as part of a novel viral mechanism of attacking a host. Accordingly, utilities of VGAM316 include diagnosis, prevention and treatment of viral infection by Vaccinia Virus. Specific functions, and accordingly utilities, of VGAM316 correlate with, and may be deduced from, the identity of the host target genes which VGAM316 binds and inhibits, and the function of these host target genes, as elaborated hereinbelow.

Nucleotide sequences of the VGAM316 precursor RNA, herein designated VGAM PRECURSOR RNA, and of the ‘diced’ VGAM316 RNA, herein designated VGAM RNA, and a schematic representation of the secondary folding of VGAM316 folded precursor RNA, herein designated VGAM FOLDED PRECURSOR RNA, of VGAM316 are further described hereinbelow with reference to Table 1.

Nucleotide sequences of host target binding sites, such as BINDING SITE-I, BINDING SITE-II and BINDING SITE-III of FIG. 1, found on VGAM316 host target RNA, and schematic representation of the complementarity of each of these host target binding sites to VGAM316 RNA, herein designated VGAM RNA, are described hereinbelow with reference to Table 2.

As mentioned hereinabove with reference to FIG. 1, a function of VGAM316 gene, herein designated VGAM is inhibition of expression of VGAM316 target genes. It is appreciated that specific functions, and accordingly utilities, of VGAM316 correlate with, and may be deduced from, the identity of the target genes which VGAM316 binds and inhibits, and the function of these target genes, as elaborated hereinbelow.

Chromosome 1 Open Reading Frame 1 (Clorf1, Accession NM_{—}001213) is a VGAM316 host target gene. Clorf1 BINDING SITE is HOST TARGET binding site found in the 5′ untranslated region of mRNA encoded by Clorf1, corresponding to a HOST TARGET binding site such as BINDING SITE I, BINDING SITE II or BINDING SITE III. Table 2 illustrates the complementarity of the nucleotide sequences of Clorf1 BINDING SITE, designated SEQ ID:809, to the nucleotide sequence of VGAM316 RNA, herein designated VGAM RNA, also designated SEQ ID:651.

A function of VGAM316 is therefore inhibition of Chromosome 1 Open Reading Frame 1 (Clorf1, Accession NM_{—}001213). Accordingly, utilities of VGAM316 include diagnosis, prevention and treatment of diseases and clinical conditions associated with Clorf1. Lecithin Retinol Acyltransferase (phosphatidyIcholine—retinol O-acyltransferase) (LRAT, Accession XM_{—}011181) is another VGAM316 host target gene. LRAT BINDING SITE is HOST TARGET binding site found in the 3′ untranslated region of mRNA encoded by LRAT, corresponding to a HOST TARGET binding site such as BINDING SITE I, BINDING SITE II or BINDING SITE III. Table 2 illustrates the complementarity of the nucleotide sequences of LRAT BINDING SITE, designated SEQ ID:2556, to the nucleotide sequence of VGAM316 RNA, herein designated VGAM RNA, also designated SEQ ID:651.

Another function of VGAM316 is therefore inhibition of Lecithin Retinol Acyltransferase (phosphatidyIcholine—retinol O-acyltransferase) (LRAT, Accession XM_{—}011181). Accordingly, utilities of VGAM316 include diagnosis, prevention and treatment of diseases and clinical conditions associated with LRAT. V-myb Myeloblastosis Viral Oncogene Homolog (avian)-like 1 (MYBL1, Accession XM_{—}034274) is another VGAM316 host target gene. MYBL1 BINDING SITE is HOST TARGET binding site found in the 3′ untranslated region of mRNA encoded by MYBL1, corresponding to a HOST TARGET binding site such as BINDING SITE I, BINDING SITE II or BINDING SITE III. Table 2 illustrates the complementarity of the nucleotide sequences of MYBL1 BINDING SITE, designated SEQ ID:2679, to the nucleotide sequence of VGAM316 RNA, herein designated VGAM RNA, also designated SEQ ID:651.

Another function of VGAM316 is therefore inhibition of V-myb Myeloblastosis Viral Oncogene Homolog (avian)-like 1 (MYBL1, Accession XM_{—}034274), a gene which could have a role in the proliferation and/or differentiation of neurogenic, spermatogenic and b-lymphoid cells. Accordingly, utilities of VGAM316 include diagnosis, prevention and treatment of diseases and clinical conditions associated with MYBL1. The function of MYBL1 and its association with various diseases and clinical conditions, has been established by previous studies, as described hereinabove with reference to VGAM51. KIAA1028 (Accession XM_{—}166324) is another VGAM316 host target gene. KIAA1028 BINDING SITE is HOST TARGET binding site found in the 3′ untranslated region of mRNA encoded by KIAA1028, corresponding to a HOST TARGET binding site such as BINDING SITE I, BINDING SITE II or BINDING SITE III. Table 2 illustrates the complementarity of the nucleotide sequences of KIAA1028 BINDING SITE, designated SEQ ID:3547, to the nucleotide sequence of VGAM316 RNA, herein designated VGAM RNA, also designated SEQ ID:651.

Another function of VGAM316 is therefore inhibition of KIAA1028 (Accession XM_{—}166324). Accordingly, utilities of VGAM316 include diagnosis, prevention and treatment of diseases and clinical conditions associated with KIAA1028. MGC2488 (Accession NM_{—}024039) is another VGAM316 host target gene. MGC2488 BINDING SITE is HOST TARGET binding site found in the 3′ untranslated region of mRNA encoded by MGC2488, corresponding to a HOST TARGET binding site such as BINDING SITE I, BINDING SITE II or BINDING SITE III. Table 2 illustrates the complementarity of the nucleotide sequences of MGC2488 BINDING SITE, designated SEQ ID:2045, to the nucleotide sequence of VGAM316 RNA, herein designated VGAM RNA, also designated SEQ ID:651.

Another function of VGAM316 is therefore inhibition of MGC2488 (Accession NM_{—}024039). Accordingly, utilities of VGAM316 include diagnosis, prevention and treatment of diseases and clinical conditions associated with MGC2488. FIG. 1 further provides a conceptual description of a novel bioinformatically detected viral gene of the present invention, referred to here as Viral Genomic Address Messenger 317 (VGAM317) viral gene, which modulates expression of respective host target genes thereof, the function and utility of which host target genes is known in the art.

VGAM317 is a novel bioinformatically detected regulatory, non protein coding, viral micro RNA (miRNA) gene. The method by which VGAM317 was detected is described hereinabove with reference to FIGS. 1-8.

VGAM317 gene, herein designated VGAM GENE, is a viral gene contained in the genome of Vaccinia Virus. VGAM317 host target gene, herein designated VGAM HOST TARGET GENE, is a human gene contained in the human genome.

VGAM317 gene encodes a VGAM317 precursor RNA, herein designated VGAM PRECURSOR RNA. Similar to other miRNA genes, and unlike most ordinary genes, VGAM317 precursor RNA does not encode a protein. A nucleotide sequence identical or highly similar to the nucleotide sequence of VGAM317 precursor RNA is designated SEQ ID:303, and is provided hereinbelow with reference to the sequence listing part. Nucleotide sequence SEQ ID:303 is located at position 6573 relative to the genome of Vaccinia Virus.

VGAM317 precursor RNA folds onto itself, forming VGAM317 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, “dices” the VGAM317 folded precursor RNA into VGAM317 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM317 RNA is designated SEQ ID:652, and is provided hereinbelow with reference to the sequence listing part.

VGAM317 host target gene, herein designated VGAM HOST TARGET GENE, encodes a corresponding messenger RNA, VGAM317 host target RNA, herein designated VGAM HOST TARGET RNA. VGAM317 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM317 RNA, herein designated VGAM RNA, binds complementarily to one or more host target binding sites located in untranslated regions of VGAM317 host target RNA, herein designated VGAM HOST TARGET RNA. This complementary binding is due to the fact that the nucleotide sequence of VGAM317 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM317 RNA, herein designated VGAM RNA, may have a different number of host target binding sites in untranslated regions of a VGAM317 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM317 RNA, herein designated VGAM RNA, to host target binding sites on VGAM317 host target RNA, herein designated VGAM HOST TARGET RNA, such as BINDING SITE I, BINDING SITE II and BINDING SITE III, inhibits translation of VGAM317 host target RNA into VGAM317 host target protein, herein designated VGAM HOST TARGET PROTEIN. VGAM host target protein is therefore outlined by a broken line.

It is appreciated that VGAM317 host target gene, herein designated VGAM HOST TARGET GENE, in fact represents a plurality of VGAM317 host target genes. The mRNA of each one of this plurality of VGAM317 host target genes comprises one or more host target binding sites, each having a nucleotide sequence which is at least partly complementary to VGAM317 RNA, herein designated VGAM RNA, and which when bound by VGAM317 RNA causes inhibition of translation of respective one or more VGAM317 host target proteins.

It is further appreciated by one skilled in the art that the mode of translational inhibition illustrated by FIG. 1 with specific reference to translational inhibition exerted by VGAM317 gene, herein designated VGAM GENE, on one or more VGAM317 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM317 is inhibition of expression of host target genes, as part of a novel viral mechanism of attacking a host. Accordingly, utilities of VGAM317 include diagnosis, prevention and treatment of viral infection by Vaccinia Virus. Specific functions, and accordingly utilities, of VGAM317 correlate with, and may be deduced from, the identity of the host target genes which VGAM317 binds and inhibits, and the function of these host target genes, as elaborated hereinbelow.

Nucleotide sequences of the VGAM317 precursor RNA, herein designated VGAM PRECURSOR RNA, and of the ‘diced’ VGAM317 RNA, herein designated VGAM RNA, and a schematic representation of the secondary folding of VGAM317 folded precursor RNA, herein designated VGAM FOLDED PRECURSOR RNA, of VGAM317 are further described hereinbelow with reference to Table 1.

Nucleotide sequences of host target binding sites, such as BINDING SITE-I, BINDING SITE-II and BINDING SITE-III of FIG. 1, found on VGAM317 host target RNA, and schematic representation of the complementarity of each of these host target binding sites to VGAM317 RNA, herein designated VGAM RNA, are described hereinbelow with reference to Table 2.

As mentioned hereinabove with reference to FIG. 1, a function of VGAM317 gene, herein designated VGAM is inhibition of expression of VGAM317 target genes. It is appreciated that specific functions, and accordingly utilities, of VGAM317 correlate with, and may be deduced from, the identity of the target genes which VGAM317 binds and inhibits, and the function of these target genes, as elaborated hereinbelow.

Chemokine (C-X3-C motif) Receptor 1 (CX3CR1, Accession XM_{—}047502) is a VGAM317 host target gene. CX3CR1 BINDING SITE is HOST TARGET binding site found in the 3′ untranslated region of mRNA encoded by CX3CR1, corresponding to a HOST TARGET binding site such as BINDING SITE I, BINDING SITE II or BINDING SITE III. Table 2 illustrates the complementarity of the nucleotide sequences of CX3CR1 BINDING SITE, designated SEQ ID:2891, to the nucleotide sequence of VGAM317 RNA, herein designated VGAM RNA, also designated SEQ ID:652.

A function of VGAM317 is therefore inhibition of Chemokine (C-X3-C motif) Receptor 1 (CX3CR1, Accession XM_{—}047502), a gene which mediates both the adhesive and migratory functions of fractalkine. Accordingly, utilities of VGAM317 include diagnosis, prevention and treatment of diseases and clinical conditions associated with CX3CR1. The function of CX3CR1 and its association with various diseases and clinical conditions, has been established by previous studies, as described hereinabove with reference to VGAM 52. Wingless-type MMTV Integration Site Family, Member 14 (WNT14, Accession NM_{—}003395) is another VGAM317 host target gene. WNT14 BINDING SITE is HOST TARGET binding site found in the 3′ untranslated region of mRNA encoded by WNT14, corresponding to a HOST TARGET binding site such as BINDING SITE I, BINDING SITE II or BINDING SITE III. Table 2 illustrates the complementarity of the nucleotide sequences of WNT14 BINDING SITE, designated SEQ ID:1014, to the nucleotide sequence of VGAM317 RNA, herein designated VGAM RNA, also designated SEQ ID:652.

Another function of VGAM317 is therefore inhibition of Wingless-type MMTV Integration Site Family, Member 14 (WNT14, Accession NM_{—}003395), a gene which is a ligand for members of the frizzled family of seven transmembrane receptors and may be a signaling molecule which affect the development of discrete regions of tissues. Accordingly, utilities of VGAM317 include diagnosis, prevention and treatment of diseases and clinical conditions associated with WNT14. The function of WNT14 and its association with various diseases and clinical conditions, has been established by previous studies, as described hereinabove with reference to VGAM52. FLJ20275 (Accession NM_{—}017737) is another VGAM317 host target gene. FLJ20275 BINDING SITE is HOST TARGET binding site found in the 3′ untranslated region of mRNA encoded by FLJ20275, corresponding to a HOST TARGET binding site such as BINDING SITE I, BINDING SITE II or BINDING SITE III. Table 2 illustrates the complementarity of the nucleotide sequences of FLJ20275 BINDING SITE, designated SEQ ID:1745, to the nucleotide sequence of VGAM317 RNA, herein designated VGAM RNA, also designated SEQ ID:652.

Another function of VGAM317 is therefore inhibition of FLJ20275 (Accession NM_{—}017737). Accordingly, utilities of VGAM317 include diagnosis, prevention and treatment of diseases and clinical conditions associated with FLJ20275. FLJ22390 (Accession NM_{—}022746) is another VGAM317 host target gene. FLJ22390 BINDING SITE is HOST TARGET binding site found in the 5′ untranslated region of mRNA encoded by FLJ22390, corresponding to a HOST TARGET binding site such as BINDING SITE I, BINDING SITE II or BINDING SITE III. Table 2 illustrates the complementarity of the nucleotide sequences of FLJ22390 BINDING SITE, designated SEQ ID:2002, to the nucleotide sequence of VGAM317 RNA, herein designated VGAM RNA, also designated SEQ ID:652.

Another function of VGAM317 is therefore inhibition of FLJ22390 (Accession NM_{—}022746). Accordingly, utilities of VGAM317 include diagnosis, prevention and treatment of diseases and clinical conditions associated with FLJ22390. FIG. 1 further provides a conceptual description of a novel bioinformatically detected viral gene of the present invention, referred to here as Viral Genomic Address Messenger 318 (VGAM318) viral gene, which modulates expression of respective host target genes thereof, the function and utility of which host target genes is known in the art.

VGAM318 is a novel bioinformatically detected regulatory, non protein coding, viral micro RNA (miRNA) gene. The method by which VGAM318 was detected is described hereinabove with reference to FIGS. 1-8.

VGAM318 gene, herein designated VGAM GENE, is a viral gene contained in the genome of Vaccinia Virus. VGAM318 host target gene, herein designated VGAM HOST TARGET GENE, is a human gene contained in the human genome.

VGAM318 gene encodes a VGAM318 precursor RNA, herein designated VGAM PRECURSOR RNA. Similar to other miRNA genes, and unlike most ordinary genes, VGAM318 precursor RNA does not encode a protein. A nucleotide sequence identical or highly similar to the nucleotide sequence of VGAM318 precursor RNA is designated SEQ ID:304, and is provided hereinbelow with reference to the sequence listing part. Nucleotide sequence SEQ ID:304 is located at position 5257 relative to the genome of Vaccinia Virus.

VGAM318 precursor RNA folds onto itself, forming VGAM318 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM318 folded precursor RNA into VGAM318 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM318 RNA is designated SEQ ID:653, and is provided hereinbelow with reference to the sequence listing part.

VGAM318 host target gene, herein designated VGAM HOST TARGET GENE, encodes a corresponding messenger RNA, VGAM318 host target RNA, herein designated VGAM HOST TARGET RNA. VGAM318 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM318 RNA, herein designated VGAM RNA, binds complementarily to one or more host target binding sites located in untranslated regions of VGAM318 host target RNA, herein designated VGAM HOST TARGET RNA. This complementary binding is due to the fact that the nucleotide sequence of VGAM318 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM318 RNA, herein designated VGAM RNA, may have a different number of host target binding sites in untranslated regions of a VGAM318 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM318 RNA, herein designated VGAM RNA, to host target binding sites on VGAM318 host target RNA, herein designated VGAM HOST TARGET RNA, such as BINDING SITE I, BINDING SITE II and BINDING SITE III, inhibits translation of VGAM318 host target RNA into VGAM318 host target protein, herein designated VGAM HOST TARGET PROTEIN. VGAM host target protein is therefore outlined by a broken line.

It is appreciated that VGAM318 host target gene, herein designated VGAM HOST TARGET GENE, in fact represents a plurality of VGAM318 host target genes. The mRNA of each one of this plurality of VGAM318 host target genes comprises one or more host target binding sites, each having a nucleotide sequence which is at least partly complementary to VGAM318 RNA, herein designated VGAM RNA, and which when bound by VGAM318 RNA causes inhibition of translation of respective one or more VGAM318 host target proteins.

It is further appreciated by one skilled in the art that the mode of translational inhibition illustrated by FIG. 1 with specific reference to translational inhibition exerted by VGAM318 gene, herein designated VGAM GENE, on one or more VGAM318 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM318 is inhibition of expression of host target genes, as part of a novel viral mechanism of attacking a host. Accordingly, utilities of VGAM318 include diagnosis, prevention and treatment of viral infection by Vaccinia Virus. Specific functions, and accordingly utilities, of VGAM318 correlate with, and may be deduced from, the identity of the host target genes which VGAM318 binds and inhibits, and the function of these host target genes, as elaborated hereinbelow.

Nucleotide sequences of the VGAM318 precursor RNA, herein designated VGAM PRECURSOR RNA, and of the ‘diced’ VGAM318 RNA, herein designated VGAM RNA, and a schematic representation of the secondary folding of VGAM318 folded precursor RNA, herein designated VGAM FOLDED PRECURSOR RNA, of VGAM318 are further described hereinbelow with reference to Table 1.

Nucleotide sequences of host target binding sites, such as BINDING SITE-I, BINDING SITE-II and BINDING SITE-III of FIG. 1, found on VGAM318 host target RNA, and schematic representation of the complementarity of each of these host target binding sites to VGAM318 RNA, herein designated VGAM RNA, are described hereinbelow with reference to Table 2.

As mentioned hereinabove with reference to FIG. 1, a function of VGAM318 gene, herein designated VGAM is inhibition of expression of VGAM318 target genes. It is appreciated that specific functions, and accordingly utilities, of VGAM318 correlate with, and may be deduced from, the identity of the target genes which VGAM318 binds and inhibits, and the function of these target genes, as elaborated hereinbelow.

LOC120864 (Accession XM_{—}058510) is a VGAM318 host target gene. LOC120864 BINDING SITE is HOST TARGET binding site found in the 5′ untranslated region of mRNA encoded by LOC120864, corresponding to a HOST TARGET binding site such as BINDING SITE I, BINDING SITE II or BINDING SITE III. Table 2 illustrates the complementarity of the nucleotide sequences of LOC120864 BINDING SITE, designated SEQ ID:2986, to the nucleotide sequence of VGAM318 RNA, herein designated VGAM RNA, also designated SEQ ID:653.

A function of VGAM318 is therefore inhibition of LOC120864 (Accession XM_{—}058510). Accordingly, utilities of VGAM318 include diagnosis, prevention and treatment of diseases and clinical conditions associated with LOC120864. FIG. 1 further provides a conceptual description of a novel bioinformatically detected viral gene of the present invention, referred to here as Viral Genomic Address Messenger 319 (VGAM319) viral gene, which modulates expression of respective host target genes thereof, the function and utility of which host target genes is known in the art.

VGAM319 is a novel bioinformatically detected regulatory, non protein coding, viral micro RNA (miRNA) gene. The method by which VGAM319 was detected is described hereinabove with reference to FIGS. 1-8.

VGAM319 gene, herein designated VGAM GENE, is a viral gene contained in the genome of Vaccinia Virus. VGAM319 host target gene, herein designated VGAM HOST TARGET GENE, is a human gene contained in the human genome.

VGAM319 gene encodes a VGAM319 precursor RNA, herein designated VGAM PRECURSOR RNA. Similar to other miRNA genes, and unlike most ordinary genes, VGAM319 precursor RNA does not encode a protein. A nucleotide sequence identical or highly similar to the nucleotide sequence of VGAM319 precursor RNA is designated SEQ ID:305, and is provided hereinbelow with reference to the sequence listing part. Nucleotide sequence SEQ ID:305 is located at position 4867 relative to the genome of Vaccinia Virus.

VGAM319 precursor RNA folds onto itself, forming VGAM319 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM319 folded precursor RNA into VGAM319 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM319 RNA is designated SEQ ID:654, and is provided hereinbelow with reference to the sequence listing part.

VGAM319 host target gene, herein designated VGAM HOST TARGET GENE, encodes a corresponding messenger RNA, VGAM319 host target RNA, herein designated VGAM HOST TARGET RNA. VGAM319 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM319 RNA, herein designated VGAM RNA, binds complementarily to one or more host target binding sites located in untranslated regions of VGAM319 host target RNA, herein designated VGAM HOST TARGET RNA. This complementary binding is due to the fact that the nucleotide sequence of VGAM319 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM319 RNA, herein designated VGAM RNA, may have a different number of host target binding sites in untranslated regions of a VGAM319 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM319 RNA, herein designated VGAM RNA, to host target binding sites on VGAM319 host target RNA, herein designated VGAM HOST TARGET RNA, such as BINDING SITE I, BINDING SITE II and BINDING SITE III, inhibits translation of VGAM319 host target RNA into VGAM319 host target protein, herein designated VGAM HOST TARGET PROTEIN. VGAM host target protein is therefore outlined by a broken line.

It is appreciated that VGAM319 host target gene, herein designated VGAM HOST TARGET GENE, in fact represents a plurality of VGAM319 host target genes. The mRNA of each one of this plurality of VGAM319 host target genes comprises one or more host target binding sites, each having a nucleotide sequence which is at least partly complementary to VGAM319 RNA, herein designated VGAM RNA, and which when bound by VGAM319 RNA causes inhibition of translation of respective one or more VGAM319 host target proteins.

It is further appreciated by one skilled in the art that the mode of translational inhibition illustrated by FIG. 1 with specific reference to translational inhibition exerted by VGAM319 gene, herein designated VGAM GENE, on one or more VGAM319 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM319 is inhibition of expression of host target genes, as part of a novel viral mechanism of attacking a host. Accordingly, utilities of VGAM319 include diagnosis, prevention and treatment of viral infection by Vaccinia Virus. Specific functions, and accordingly utilities, of VGAM319 correlate with, and may be deduced from, the identity of the host target genes which VGAM319 binds and inhibits, and the function of these host target genes, as elaborated hereinbelow.

Nucleotide sequences of the VGAM319 precursor RNA, herein designated VGAM PRECURSOR RNA, and of the ‘diced’ VGAM319 RNA, herein designated VGAM RNA, and a schematic representation of the secondary folding of VGAM319 folded precursor RNA, herein designated VGAM FOLDED PRECURSOR RNA, of VGAM319 are further described hereinbelow with reference to Table 1.

Nucleotide sequences of host target binding sites, such as BINDING SITE-I, BINDING SITE-II and BINDING SITE-III of FIG. 1, found on VGAM319 host target RNA, and schematic representation of the complementarity of each of these host target binding sites to VGAM319 RNA, herein designated VGAM RNA, are described hereinbelow with reference to Table 2.

As mentioned hereinabove with reference to FIG. 1, a function of VGAM319 gene, herein designated VGAM is inhibition of expression of VGAM319 target genes. It is appreciated that specific functions, and accordingly utilities, of VGAM319 correlate with, and may be deduced from, the identity of the target genes which VGAM319 binds and inhibits, and the function of these target genes, as elaborated hereinbelow.

KIAA0626 (Accession NM_{—}021647) is a VGAM319 host target gene. KIAA0626 BINDING SITE is HOST TARGET binding site found in the 3′ untranslated region of mRNA encoded by KIAA0626, corresponding to a HOST TARGET binding site such as BINDING SITE I, BINDING SITE II or BINDING SITE III. Table 2 illustrates the complementarity of the nucleotide sequences of KIAA0626 BINDING SITE, designated SEQ ID:1954, to the nucleotide sequence of VGAM319 RNA, herein designated VGAM RNA, also designated SEQ ID:654.

A function of VGAM319 is therefore inhibition of KIAA0626 (Accession NM_{—}021647). Accordingly, utilities of VGAM319 include diagnosis, prevention and treatment of diseases and clinical conditions associated with KIAA0626. KIAA1239 (Accession XM_{—}049078) is another VGAM319 host target gene. KIAA1239 BINDING SITE is HOST TARGET binding site found in the 3′ untranslated region of mRNA encoded by KIAA1239, corresponding to a HOST TARGET binding site such as BINDING SITE I, BINDING SITE II or BINDING SITE III. Table 2 illustrates the complementarity of the nucleotide sequences of KIAA1239 BINDING SITE, designated SEQ ID:2914, to the nucleotide sequence of VGAM319 RNA, herein designated VGAM RNA, also designated SEQ ID:654.

Another function of VGAM319 is therefore inhibition of KIAA1239 (Accession XM_{—}049078). Accordingly, utilities of VGAM319 include diagnosis, prevention and treatment of diseases and clinical conditions associated with KIAA1239. LOC201475 (Accession XM_{—}113967) is another VGAM319 host target gene. LOC201475 BINDING SITE is HOST TARGET binding site found in the 5′ untranslated region of mRNA encoded by LOC201475, corresponding to a HOST TARGET binding site such as BINDING SITE I, BINDING SITE II or BINDING SITE III. Table 2 illustrates the complementarity of the nucleotide sequences of LOC201475 BINDING SITE, designated SEQ ID:3428, to the nucleotide sequence of VGAM319 RNA, herein designated VGAM RNA, also designated SEQ ID:654.

Another function of VGAM319 is therefore inhibition of LOC201475 (Accession XM_{—}113967). Accordingly, utilities of VGAM319 include diagnosis, prevention and treatment of diseases and clinical conditions associated with LOC201475. FIG. 1 further provides a conceptual description of a novel bioinformatically detected viral gene of the present invention, referred to here as Viral Genomic Address Messenger 320 (VGAM320) viral gene, which modulates expression of respective host target genes thereof, the function and utility of which host target genes is known in the art.

VGAM320 is a novel bioinformatically detected regulatory, non protein coding, viral micro RNA (miRNA) gene. The method by which VGAM320 was detected is described hereinabove with reference to FIGS. 1-8.

VGAM320 gene, herein designated VGAM GENE, is a viral gene contained in the genome of Vaccinia Virus. VGAM320 host target gene, herein designated VGAM HOST TARGET GENE, is a human gene contained in the human genome.

VGAM320 gene encodes a VGAM320 precursor RNA, herein designated VGAM PRECURSOR RNA. Similar to other miRNA genes, and unlike most ordinary genes, VGAM320 precursor RNA does not encode a protein. A nucleotide sequence identical or highly similar to the nucleotide sequence of VGAM320 precursor RNA is designated SEQ ID:306, and is provided hereinbelow with reference to the sequence listing part. Nucleotide sequence SEQ ID:306 is located at position 190678 relative to the genome of Vaccinia Virus.

VGAM320 precursor RNA folds onto itself, forming VGAM320 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM320 folded precursor RNA into VGAM320 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 83%) nucleotide sequence of VGAM320 RNA is designated SEQ ID:655, and is provided hereinbelow with reference to the sequence listing part.

VGAM320 host target gene, herein designated VGAM HOST TARGET GENE, encodes a corresponding messenger RNA, VGAM320 host target RNA, herein designated VGAM HOST TARGET RNA. VGAM320 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM320 RNA, herein designated VGAM RNA, binds complementarily to one or more host target binding sites located in untranslated regions of VGAM320 host target RNA, herein designated VGAM HOST TARGET RNA. This complementary binding is due to the fact that the nucleotide sequence of VGAM320 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM320 RNA, herein designated VGAM RNA, may have a different number of host target binding sites in untranslated regions of a VGAM320 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM320 RNA, herein designated VGAM RNA, to host target binding sites on VGAM320 host target RNA, herein designated VGAM HOST TARGET RNA, such as BINDING SITE I, BINDING SITE II and BINDING SITE III, inhibits translation of VGAM320 host target RNA into VGAM320 host target protein, herein designated VGAM HOST TARGET PROTEIN. VGAM host target protein is therefore outlined by a broken line.

It is appreciated that VGAM320 host target gene, herein designated VGAM HOST TARGET GENE, in fact represents a plurality of VGAM320 host target genes. The mRNA of each one of this plurality of VGAM320 host target genes comprises one or more host target binding sites, each having a nucleotide sequence which is at least partly complementary to VGAM320 RNA, herein designated VGAM RNA, and which when bound by VGAM320 RNA causes inhibition of translation of respective one or more VGAM320 host target proteins.

It is further appreciated by one skilled in the art that the mode of translational inhibition illustrated by FIG. 1 with specific reference to translational inhibition exerted by VGAM320 gene, herein designated VGAM GENE, on one or more VGAM320 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM320 is inhibition of expression of host target genes, as part of a novel viral mechanism of attacking a host. Accordingly, utilities of VGAM320 include diagnosis, prevention and treatment of viral infection by Vaccinia Virus. Specific functions, and accordingly utilities, of VGAM320 correlate with, and may be deduced from, the identity of the host target genes which VGAM320 binds and inhibits, and the function of these host target genes, as elaborated hereinbelow.

Nucleotide sequences of the VGAM320 precursor RNA, herein designated VGAM PRECURSOR RNA, and of the ‘diced’ VGAM320 RNA, herein designated VGAM RNA, and a schematic representation of the secondary folding of VGAM320 folded precursor RNA, herein designated VGAM FOLDED PRECURSOR RNA, of VGAM320 are further described hereinbelow with reference to Table 1.

Nucleotide sequences of host target binding sites, such as BINDING SITE-I, BINDING SITE-II and BINDING SITE-III of FIG. 1, found on VGAM320 host target RNA, and schematic representation of the complementarity of each of these host target binding sites to VGAM320 RNA, herein designated VGAM RNA, are described hereinbelow with reference to Table 2.

As mentioned hereinabove with reference to FIG. 1, a function of VGAM320 gene, herein designated VGAM is inhibition of expression of VGAM320 target genes. It is appreciated that specific functions, and accordingly utilities, of VGAM320 correlate with, and may be deduced from, the identity of the target genes which VGAM320 binds and inhibits, and the function of these target genes, as elaborated hereinbelow.

Calpain 2, (m/II) Large Subunit (CAPN2, Accession NM_{—}001748) is a VGAM320 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM320 RNA, herein designated VGAM RNA, also designated SEQ ID:655.

A function of VGAM320 is therefore inhibition of Calpain 2, (m/II) Large Subunit (CAPN2, Accession NM_{—}001748). Accordingly, utilities of VGAM320 include diagnosis, prevention and treatment of diseases and clinical conditions associated with CAPN2. Dishevelled Associated Activator of Morphogenesis 2 (DAAM2, Accession XM_{—}166434) is another VGAM320 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM320 RNA, herein designated VGAM RNA, also designated SEQ ID:655.

Another function of VGAM320 is therefore inhibition of Dishevelled Associated Activator of Morphogenesis 2 (DAAM2, Accession XM_{—}166434), a gene which controls cell polarity and movement during development. Accordingly, utilities of VGAM320 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM320 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM320 RNA, herein designated VGAM RNA, also designated SEQ ID:655.

Another function of VGAM320 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM320 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM320 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM320 RNA, herein designated VGAM RNA, also designated SEQ ID:655.

Another function of VGAM320 is therefore inhibition of KIAA0475 (Accession NM_{—}014864). Accordingly, utilities of VGAM320 include diagnosis, prevention and treatment of diseases and clinical conditions associated with KIAA0475. LOC222671 (Accession XM_{—}167094) is another VGAM320 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM320 RNA, herein designated VGAM RNA, also designated SEQ ID:655.

Another function of VGAM320 is therefore inhibition of LOC222671 (Accession XM_{—}167094). Accordingly, utilities of VGAM320 include diagnosis, prevention and treatment of diseases and clinical conditions associated with LOC222671. LOC254901 (Accession XM_{—}171878) is another VGAM320 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM320 RNA, herein designated VGAM RNA, also designated SEQ ID:655.

Another function of VGAM320 is therefore inhibition of LOC254901 (Accession XM_{—}171878). Accordingly, utilities of VGAM320 include diagnosis, prevention and treatment of diseases and clinical conditions associated with LOC254901. LOC257615 (Accession XM_{—}175293) is another VGAM320 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM320 RNA, herein designated VGAM RNA, also designated SEQ ID:655.

Another function of VGAM320 is therefore inhibition of LOC257615 (Accession XM_{—}175293). Accordingly, utilities of VGAM320 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 321 (VGAM321) viral gene, which modulates expression of respective host target genes thereof, the function and utility of which host target genes is known in the art.

VGAM321 is a novel bioinformatically detected regulatory, non protein coding, viral micro RNA (miRNA) gene. The method by which VGAM321 was detected is described hereinabove with reference to FIGS. 1-8.

VGAM321 gene, herein designated VGAM GENE, is a viral gene contained in the genome of Vaccinia Virus. VGAM321 host target gene, herein designated VGAM HOST TARGET GENE, is a human gene contained in the human genome.

VGAM321 gene encodes a VGAM321 precursor RNA, herein designated VGAM PRECURSOR RNA. Similar to other miRNA genes, and unlike most ordinary genes, VGAM321 precursor RNA does not encode a protein. A nucleotide sequence identical or highly similar to the nucleotide sequence of VGAM321 precursor RNA is designated SEQ ID:307, and is provided hereinbelow with reference to the sequence listing part. Nucleotide sequence SEQ ID:307 is located at position 188927 relative to the genome of Vaccinia Virus.

VGAM321 precursor RNA folds onto itself, forming VGAM321 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM321 folded precursor RNA into VGAM321 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM321 RNA is designated SEQ ID:656, and is provided hereinbelow with reference to the sequence listing part.

VGAM321 host target gene, herein designated VGAM HOST TARGET GENE, encodes a corresponding messenger RNA, VGAM321 host target RNA, herein designated VGAM HOST TARGET RNA. VGAM321 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM321 RNA, herein designated VGAM RNA, binds complementarily to one or more host target binding sites located in untranslated regions of VGAM321 host target RNA, herein designated VGAM HOST TARGET RNA. This complementary binding is due to the fact that the nucleotide sequence of VGAM321 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM321 RNA, herein designated VGAM RNA, may have a different number of host target binding sites in untranslated regions of a VGAM321 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM321 RNA, herein designated VGAM RNA, to host target binding sites on VGAM321 host target RNA, herein designated VGAM HOST TARGET RNA, such as BINDING SITE I, BINDING SITE II and BINDING SITE III, inhibits translation of VGAM321 host target RNA into VGAM321 host target protein, herein designated VGAM HOST TARGET PROTEIN. VGAM host target protein is therefore outlined by a broken line.

It is appreciated that VGAM321 host target gene, herein designated VGAM HOST TARGET GENE, in fact represents a plurality of VGAM321 host target genes. The mRNA of each one of this plurality of VGAM321 host target genes comprises one or more host target binding sites, each having a nucleotide sequence which is at least partly complementary to VGAM321 RNA, herein designated VGAM RNA, and which when bound by VGAM321 RNA causes inhibition of translation of respective one or more VGAM321 host target proteins.

It is further appreciated by one skilled in the art that the mode of translational inhibition illustrated by FIG. 1 with specific reference to translational inhibition exerted by VGAM321 gene, herein designated VGAM GENE, on one or more VGAM321 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM321 is inhibition of expression of host target genes, as part of a novel viral mechanism of attacking a host. Accordingly, utilities of VGAM321 include diagnosis, prevention and treatment of viral infection by Vaccinia Virus. Specific functions, and accordingly utilities, of VGAM321 correlate with, and may be deduced from, the identity of the host target genes which VGAM321 binds and inhibits, and the function of these host target genes, as elaborated hereinbelow.

Nucleotide sequences of the VGAM321 precursor RNA, herein designated VGAM PRECURSOR RNA, and of the ‘diced’ VGAM321 RNA, herein designated VGAM RNA, and a schematic representation of the secondary folding of VGAM321 folded precursor RNA, herein designated VGAM FOLDED PRECURSOR RNA, of VGAM321 are further described hereinbelow with reference to Table 1.

Nucleotide sequences of host target binding sites, such as BINDING SITE-I, BINDING SITE-II and BINDING SITE-III of FIG. 1, found on VGAM321 host target RNA, and schematic representation of the complementarity of each of these host target binding sites to VGAM321 RNA, herein designated VGAM RNA, are described hereinbelow with reference to Table 2.

As mentioned hereinabove with reference to FIG. 1, a function of VGAM321 gene, herein designated VGAM is inhibition of expression of VGAM321 target genes. It is appreciated that specific functions, and accordingly utilities, of VGAM321 correlate with, and may be deduced from, the identity of the target genes which VGAM321 binds and inhibits, and the function of these target genes, as elaborated hereinbelow.

Calpain 2, (m/II) Large Subunit (CAPN2, Accession NM_{—}001748) is a VGAM321 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM321 RNA, herein designated VGAM RNA, also designated SEQ ID:656.

A function of VGAM321 is therefore inhibition of Calpain 2, (m/II) Large Subunit (CAPN2, Accession NM_{—}001748). Accordingly, utilities of VGAM321 include diagnosis, prevention and treatment of diseases and clinical conditions associated with CAPN2. Dishevelled Associated Activator of Morphogenesis 2 (DAAM2, Accession XM_{—}166434) is another VGAM321 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM321 RNA, herein designated VGAM RNA, also designated SEQ ID:656.

Another function of VGAM321 is therefore inhibition of Dishevelled Associated Activator of Morphogenesis 2 (DAAM2, Accession XM_{—}166434), a gene which controls cell polarity and movement during development. Accordingly, utilities of VGAM321 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM321 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM321 RNA, herein designated VGAM RNA, also designated SEQ ID:656.

Another function of VGAM321 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM321 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM321 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM321 RNA, herein designated VGAM RNA, also designated SEQ ID:656.

Another function of VGAM321 is therefore inhibition of KIAA0475 (Accession NM_{—}014864). Accordingly, utilities of VGAM321 include diagnosis, prevention and treatment of diseases and clinical conditions associated with KIAA0475. LOC222671 (Accession XM_{—}167094) is another VGAM321 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM321 RNA, herein designated VGAM RNA, also designated SEQ ID:656.

Another function of VGAM321 is therefore inhibition of LOC222671 (Accession XM_{—}167094). Accordingly, utilities of VGAM321 include diagnosis, prevention and treatment of diseases and clinical conditions associated with LOC222671. LOC254901 (Accession XM_{—}171878) is another VGAM321 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM321 RNA, herein designated VGAM RNA, also designated SEQ ID:656.

Another function of VGAM321 is therefore inhibition of LOC254901 (Accession XM_{—}171878). Accordingly, utilities of VGAM321 include diagnosis, prevention and treatment of diseases and clinical conditions associated with LOC254901. LOC257615 (Accession XM_{—}175293) is another VGAM321 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM321 RNA, herein designated VGAM RNA, also designated SEQ ID:656.

Another function of VGAM321 is therefore inhibition of LOC257615 (Accession XM_{—}175293). Accordingly, utilities of VGAM321 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 322 (VGAM322) viral gene, which modulates expression of respective host target genes thereof, the function and utility of which host target genes is known in the art.

VGAM322 is a novel bioinformatically detected regulatory, non protein coding, viral micro RNA (miRNA) gene. The method by which VGAM322 was detected is described hereinabove with reference to FIGS. 1-8.

VGAM322 gene, herein designated VGAM GENE, is a viral gene contained in the genome of Vaccinia Virus. VGAM322 host target gene, herein designated VGAM HOST TARGET GENE, is a human gene contained in the human genome.

VGAM322 gene encodes a VGAM322 precursor RNA, herein designated VGAM PRECURSOR RNA. Similar to other miRNA genes, and unlike most ordinary genes, VGAM322 precursor RNA does not encode a protein. A nucleotide sequence identical or highly similar to the nucleotide sequence of VGAM322 precursor RNA is designated SEQ ID:308, and is provided hereinbelow with reference to the sequence listing part. Nucleotide sequence SEQ ID:308 is located at position 188927 relative to the genome of Vaccinia Virus.

VGAM322 precursor RNA folds onto itself, forming VGAM322 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM322 folded precursor RNA into VGAM322 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM322 RNA is designated SEQ ID:657, and is provided hereinbelow with reference to the sequence listing part.

VGAM322 host target gene, herein designated VGAM HOST TARGET GENE, encodes a corresponding messenger RNA, VGAM322 host target RNA, herein designated VGAM HOST TARGET RNA. VGAM322 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM322 RNA, herein designated VGAM RNA, binds complementarily to one or more host target binding sites located in untranslated regions of VGAM322 host target RNA, herein designated VGAM HOST TARGET RNA. This complementary binding is due to the fact that the nucleotide sequence of VGAM322 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM322 RNA, herein designated VGAM RNA, may have a different number of host target binding sites in untranslated regions of a VGAM322 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM322 RNA, herein designated VGAM RNA, to host target binding sites on VGAM322 host target RNA, herein designated VGAM HOST TARGET RNA, such as BINDING SITE I, BINDING SITE II and BINDING SITE III, inhibits translation of VGAM322 host target RNA into VGAM322 host target protein, herein designated VGAM HOST TARGET PROTEIN. VGAM host target protein is therefore outlined by a broken line.

It is appreciated that VGAM322 host target gene, herein designated VGAM HOST TARGET GENE, in fact represents a plurality of VGAM322 host target genes. The mRNA of each one of this plurality of VGAM322 host target genes comprises one or more host target binding sites, each having a nucleotide sequence which is at least partly complementary to VGAM322 RNA, herein designated VGAM RNA, and which when bound by VGAM322 RNA causes inhibition of translation of respective one or more VGAM322 host target proteins.

It is further appreciated by one skilled in the art that the mode of translational inhibition illustrated by FIG. 1 with specific reference to translational inhibition exerted by VGAM322 gene, herein designated VGAM GENE, on one or more VGAM322 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM322 is inhibition of expression of host target genes, as part of a novel viral mechanism of attacking a host. Accordingly, utilities of VGAM322 include diagnosis, prevention and treatment of viral infection by Vaccinia Virus. Specific functions, and accordingly utilities, of VGAM322 correlate with, and may be deduced from, the identity of the host target genes which VGAM322 binds and inhibits, and the function of these host target genes, as elaborated hereinbelow.

Nucleotide sequences of the VGAM322 precursor RNA, herein designated VGAM PRECURSOR RNA, and of the ‘diced’ VGAM322 RNA, herein designated VGAM RNA, and a schematic representation of the secondary folding of VGAM322 folded precursor RNA, herein designated VGAM FOLDED PRECURSOR RNA, of VGAM322 are further described hereinbelow with reference to Table 1.

Nucleotide sequences of host target binding sites, such as BINDING SITE-I, BINDING SITE-II and BINDING SITE-III of FIG. 1, found on VGAM322 host target RNA, and schematic representation of the complementarity of each of these host target binding sites to VGAM322 RNA, herein designated VGAM RNA, are described hereinbelow with reference to Table 2.

As mentioned hereinabove with reference to FIG. 1, a function of VGAM322 gene, herein designated VGAM is inhibition of expression of VGAM322 target genes. It is appreciated that specific functions, and accordingly utilities, of VGAM322 correlate with, and may be deduced from, the identity of the target genes which VGAM322 binds and inhibits, and the function of these target genes, as elaborated hereinbelow.

Calpain 2, (m/II) Large Subunit (CAPN2, Accession NM_{—}001748) is a VGAM322 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM322 RNA, herein designated VGAM RNA, also designated SEQ ID:657.

A function of VGAM322 is therefore inhibition of Calpain 2, (m/II) Large Subunit (CAPN2, Accession NM_{—}001748). Accordingly, utilities of VGAM322 include diagnosis, prevention and treatment of diseases and clinical conditions associated with CAPN2. Dishevelled Associated Activator of Morphogenesis 2 (DAAM2, Accession XM_{—}166434) is another VGAM322 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM322 RNA, herein designated VGAM RNA, also designated SEQ ID:657.

Another function of VGAM322 is therefore inhibition of Dishevelled Associated Activator of Morphogenesis 2 (DAAM2, Accession XM_{—}166434), a gene which controls cell polarity and movement during development. Accordingly, utilities of VGAM322 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM322 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM322 RNA, herein designated VGAM RNA, also designated SEQ ID:657.

Another function of VGAM322 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM322 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM322 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM322 RNA, herein designated VGAM RNA, also designated SEQ ID:657.

Another function of VGAM322 is therefore inhibition of KIAA0475 (Accession NM_{—}014864). Accordingly, utilities of VGAM322 include diagnosis, prevention and treatment of diseases and clinical conditions associated with KIAA0475. LOC222671 (Accession XM_{—}167094) is another VGAM322 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM322 RNA, herein designated VGAM RNA, also designated SEQ ID:657.

Another function of VGAM322 is therefore inhibition of LOC222671 (Accession XM_{—}167094). Accordingly, utilities of VGAM322 include diagnosis, prevention and treatment of diseases and clinical conditions associated with LOC222671. LOC254901 (Accession XM_{—}171878) is another VGAM322 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM322 RNA, herein designated VGAM RNA, also designated SEQ ID:657.

Another function of VGAM322 is therefore inhibition of LOC254901 (Accession XM_{—}171878). Accordingly, utilities of VGAM322 include diagnosis, prevention and treatment of diseases and clinical conditions associated with LOC254901. LOC257615 (Accession XM_{—}175293) is another VGAM322 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM322 RNA, herein designated VGAM RNA, also designated SEQ ID:657.

Another function of VGAM322 is therefore inhibition of LOC257615 (Accession XM_{—}175293). Accordingly, utilities of VGAM322 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 323 (VGAM323) viral gene, which modulates expression of respective host target genes thereof, the function and utility of which host target genes is known in the art.

VGAM323 is a novel bioinformatically detected regulatory, non protein coding, viral micro RNA (miRNA) gene. The method by which VGAM323 was detected is described hereinabove with reference to FIGS. 1-8.

VGAM323 gene, herein designated VGAM GENE, is a viral gene contained in the genome of Vaccinia Virus. VGAM323 host target gene, herein designated VGAM HOST TARGET GENE, is a human gene contained in the human genome.

VGAM323 gene encodes a VGAM323 precursor RNA, herein designated VGAM PRECURSOR RNA. Similar to other miRNA genes, and unlike most ordinary genes, VGAM323 precursor RNA does not encode a protein. A nucleotide sequence identical or highly similar to the nucleotide sequence of VGAM323 precursor RNA is designated SEQ ID:309, and is provided hereinbelow with reference to the sequence listing part. Nucleotide sequence SEQ ID:309 is located at position 188927 relative to the genome of Vaccinia Virus.

VGAM323 precursor RNA folds onto itself, forming VGAM323 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM323 folded precursor RNA into VGAM323 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM323 RNA is designated SEQ ID:658, and is provided hereinbelow with reference to the sequence listing part.

VGAM323 host target gene, herein designated VGAM HOST TARGET GENE, encodes a corresponding messenger RNA, VGAM323 host target RNA, herein designated VGAM HOST TARGET RNA. VGAM323 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM323 RNA, herein designated VGAM RNA, binds complementarily to one or more host target binding sites located in untranslated regions of VGAM323 host target RNA, herein designated VGAM HOST TARGET RNA. This complementary binding is due to the fact that the nucleotide sequence of VGAM323 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM323 RNA, herein designated VGAM RNA, may have a different number of host target binding sites in untranslated regions of a VGAM323 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′UTR and 5′UTR regions.

The complementary binding of VGAM323 RNA, herein designated VGAM RNA, to host target binding sites on VGAM323 host target RNA, herein designated VGAM HOST TARGET RNA, such as BINDING SITE I, BINDING SITE II and BINDING SITE III, inhibits translation of VGAM323 host target RNA into VGAM323 host target protein, herein designated VGAM HOST TARGET PROTEIN. VGAM host target protein is therefore outlined by a broken line.

It is appreciated that VGAM323 host target gene, herein designated VGAM HOST TARGET GENE, in fact represents a plurality of VGAM323 host target genes. The mRNA of each one of this plurality of VGAM323 host target genes comprises one or more host target binding sites, each having a nucleotide sequence which is at least partly complementary to VGAM323 RNA, herein designated VGAM RNA, and which when bound by VGAM323 RNA causes inhibition of translation of respective one or more VGAM323 host target proteins.

It is further appreciated by one skilled in the art that the mode of translational inhibition illustrated by FIG. 1 with specific reference to translational inhibition exerted by VGAM323 gene, herein designated VGAM GENE, on one or more VGAM323 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM323 is inhibition of expression of host target genes, as part of a novel viral mechanism of attacking a host. Accordingly, utilities of VGAM323 include diagnosis, prevention and treatment of viral infection by Vaccinia Virus. Specific functions, and accordingly utilities, of VGAM323 correlate with, and may be deduced from, the identity of the host target genes which VGAM323 binds and inhibits, and the function of these host target genes, as elaborated hereinbelow.

Nucleotide sequences of the VGAM323 precursor RNA, herein designated VGAM PRECURSOR RNA, and of the ‘diced’ VGAM323 RNA, herein designated VGAM RNA, and a schematic representation of the secondary folding of VGAM323 folded precursor RNA, herein designated VGAM FOLDED PRECURSOR RNA, of VGAM323 are further described hereinbelow with reference to Table 1.

Nucleotide sequences of host target binding sites, such as BINDING SITE-I, BINDING SITE-II and BINDING SITE-III of FIG. 1, found on VGAM323 host target RNA, and schematic representation of the complementarity of each of these host target binding sites to VGAM323 RNA, herein designated VGAM RNA, are described hereinbelow with reference to Table 2.

As mentioned hereinabove with reference to FIG. 1, a function of VGAM323 gene, herein designated VGAM is inhibition of expression of VGAM323 target genes. It is appreciated that specific functions, and accordingly utilities, of VGAM323 correlate with, and may be deduced from, the identity of the target genes which VGAM323 binds and inhibits, and the function of these target genes, as elaborated hereinbelow.

Calpain 2, (m/II) Large Subunit (CAPN2, Accession NM_{—}001748) is a VGAM323 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM323 RNA, herein designated VGAM RNA, also designated SEQ ID:658.

A function of VGAM323 is therefore inhibition of Calpain 2, (m/II) Large Subunit (CAPN2, Accession NM_{—}001748). Accordingly, utilities of VGAM323 include diagnosis, prevention and treatment of diseases and clinical conditions associated with CAPN2. Dishevelled Associated Activator of Morphogenesis 2 (DAAM2, Accession XM_{—}166434) is another VGAM323 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM323 RNA, herein designated VGAM RNA, also designated SEQ ID:658.

Another function of VGAM323 is therefore inhibition of Dishevelled Associated Activator of Morphogenesis 2 (DAAM2, Accession XM_{—}166434), a gene which controls cell polarity and movement during development. Accordingly, utilities of VGAM323 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM323 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM323 RNA, herein designated VGAM RNA, also designated SEQ ID:658.

Another function of VGAM323 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM323 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM323 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM323 RNA, herein designated VGAM RNA, also designated SEQ ID:658.

Another function of VGAM323 is therefore inhibition of KIAA0475 (Accession NM_{—}014864). Accordingly, utilities of VGAM323 include diagnosis, prevention and treatment of diseases and clinical conditions associated with KIAA0475. LOC222671 (Accession XM_{—}167094) is another VGAM323 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM323 RNA, herein designated VGAM RNA, also designated SEQ ID:658.

Another function of VGAM323 is therefore inhibition of LOC222671 (Accession XM_{—}167094). Accordingly, utilities of VGAM323 include diagnosis, prevention and treatment of diseases and clinical conditions associated with LOC222671. LOC254901 (Accession XM_{—}171878) is another VGAM323 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM323 RNA, herein designated VGAM RNA, also designated SEQ ID:658.

Another function of VGAM323 is therefore inhibition of LOC254901 (Accession XM_{—}171878). Accordingly, utilities of VGAM323 include diagnosis, prevention and treatment of diseases and clinical conditions associated with LOC254901. LOC257615 (Accession XM_{—}175293) is another VGAM323 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM323 RNA, herein designated VGAM RNA, also designated SEQ ID:658.

Another function of VGAM323 is therefore inhibition of LOC257615 (Accession XM_{—}175293). Accordingly, utilities of VGAM323 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 324 (VGAM324) viral gene, which modulates expression of respective host target genes thereof, the function and utility of which host target genes is known in the art.

VGAM324 is a novel bioinformatically detected regulatory, non protein coding, viral micro RNA (miRNA) gene. The method by which VGAM324 was detected is described hereinabove with reference to FIGS. 1-8.

VGAM324 gene, herein designated VGAM GENE, is a viral gene contained in the genome of Vaccinia Virus. VGAM324 host target gene, herein designated VGAM HOST TARGET GENE, is a human gene contained in the human genome.

VGAM324 gene encodes a VGAM324 precursor RNA, herein designated VGAM PRECURSOR RNA. Similar to other miRNA genes, and unlike most ordinary genes, VGAM324 precursor RNA does not encode a protein. A nucleotide sequence identical or highly similar to the nucleotide sequence of VGAM324 precursor RNA is designated SEQ ID:310, and is provided hereinbelow with reference to the sequence listing part. Nucleotide sequence SEQ ID:310 is located at position 188927 relative to the genome of Vaccinia Virus.

VGAM324 precursor RNA folds onto itself, forming VGAM324 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM324 folded precursor RNA into VGAM324 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM324 RNA is designated SEQ ID:659, and is provided hereinbelow with reference to the sequence listing part.

VGAM324 host target gene, herein designated VGAM HOST TARGET GENE, encodes a corresponding messenger RNA, VGAM324 host target RNA, herein designated VGAM HOST TARGET RNA. VGAM324 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM324 RNA, herein designated VGAM RNA, binds complementarily to one or more host target binding sites located in untranslated regions of VGAM324 host target RNA, herein designated VGAM HOST TARGET RNA. This complementary binding is due to the fact that the nucleotide sequence of VGAM324 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM324 RNA, herein designated VGAM RNA, may have a different number of host target binding sites in untranslated regions of a VGAM324 host target RNA, herein designated VGAM HOST TARGET RNA. It is further appreciated that while FIG. 1 depicts host target binding sites in the 3′UTR region, this is meant as an example only—these host target binding sites may be located in the 3′ UTR region, the 5′UTR region, or in both 3′ UTR and 5′UTR regions.

The complementary binding of VGAM324 RNA, herein designated VGAM RNA, to host target binding sites on VGAM324 host target RNA, herein designated VGAM HOST TARGET RNA, such as BINDING SITE I, BINDING SITE II and BINDING SITE III, inhibits translation of VGAM324 host target RNA into VGAM324 host target protein, herein designated VGAM HOST TARGET PROTEIN. VGAM host target protein is therefore outlined by a broken line.

It is appreciated that VGAM324 host target gene, herein designated VGAM HOST TARGET GENE, in fact represents a plurality of VGAM324 host target genes. The mRNA of each one of this plurality of VGAM324 host target genes comprises one or more host target binding sites, each having a nucleotide sequence which is at least partly complementary to VGAM324 RNA, herein designated VGAM RNA, and which when bound by VGAM324 RNA causes inhibition of translation of respective one or more VGAM324 host target proteins.

It is further appreciated by one skilled in the art that the mode of translational inhibition illustrated by FIG. 1 with specific reference to translational inhibition exerted by VGAM324 gene, herein designated VGAM GENE, on one or more VGAM324 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM324 is inhibition of expression of host target genes, as part of a novel viral mechanism of attacking a host. Accordingly, utilities of VGAM324 include diagnosis, prevention and treatment of viral infection by Vaccinia Virus. Specific functions, and accordingly utilities, of VGAM324 correlate with, and may be deduced from, the identity of the host target genes which VGAM324 binds and inhibits, and the function of these host target genes, as elaborated hereinbelow.

Nucleotide sequences of the VGAM324 precursor RNA, herein designated VGAM PRECURSOR RNA, and of the ‘diced’ VGAM324 RNA, herein designated VGAM RNA, and a schematic representation of the secondary folding of VGAM324 folded precursor RNA, herein designated VGAM FOLDED PRECURSOR RNA, of VGAM324 are further described hereinbelow with reference to Table 1.

Nucleotide sequences of host target binding sites, such as BINDING SITE-I, BINDING SITE-II and BINDING SITE-III of FIG. 1, found on VGAM324 host target RNA, and schematic representation of the complementarity of each of these host target binding sites to VGAM324 RNA, herein designated VGAM RNA, are described hereinbelow with reference to Table 2.

As mentioned hereinabove with reference to FIG. 1, a function of VGAM324 gene, herein designated VGAM is inhibition of expression of VGAM324 target genes. It is appreciated that specific functions, and accordingly utilities, of VGAM324 correlate with, and may be deduced from, the identity of the target genes which VGAM324 binds and inhibits, and the function of these target genes, as elaborated hereinbelow.

Calpain 2, (m/II) Large Subunit (CAPN2, Accession NM_{—}001748) is a VGAM324 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM324 RNA, herein designated VGAM RNA, also designated SEQ ID:659.

A function of VGAM324 is therefore inhibition of Calpain 2, (m/II) Large Subunit (CAPN2, Accession NM_{—}001748). Accordingly, utilities of VGAM324 include diagnosis, prevention and treatment of diseases and clinical conditions associated with CAPN2. Dishevelled Associated Activator of Morphogenesis 2 (DAAM2, Accession XM_{—}166434) is another VGAM324 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM324 RNA, herein designated VGAM RNA, also designated SEQ ID:659.

Another function of VGAM324 is therefore inhibition of Dishevelled Associated Activator of Morphogenesis 2 (DAAM2, Accession XM_{—}166434), a gene which controls cell polarity and movement during development. Accordingly, utilities of VGAM324 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM324 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM324 RNA, herein designated VGAM RNA, also designated SEQ ID:659.

Another function of VGAM324 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM324 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM324 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM324 RNA, herein designated VGAM RNA, also designated SEQ ID:659.

Another function of VGAM324 is therefore inhibition of KIAA0475 (Accession NM_{—}014864). Accordingly, utilities of VGAM324 include diagnosis, prevention and treatment of diseases and clinical conditions associated with KIAA0475. LOC222671 (Accession XM_{—}167094) is another VGAM324 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM324 RNA, herein designated VGAM RNA, also designated SEQ ID:659.

Another function of VGAM324 is therefore inhibition of LOC222671 (Accession XM_{—}167094). Accordingly, utilities of VGAM324 include diagnosis, prevention and treatment of diseases and clinical conditions associated with LOC222671. LOC254901 (Accession XM_{—}171878) is another VGAM324 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM324 RNA, herein designated VGAM RNA, also designated SEQ ID:659.

Another function of VGAM324 is therefore inhibition of LOC254901 (Accession XM_{—}171878). Accordingly, utilities of VGAM324 include diagnosis, prevention and treatment of diseases and clinical conditions associated with LOC254901. LOC257615 (Accession XM_{—}175293) is another VGAM324 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM324 RNA, herein designated VGAM RNA, also designated SEQ ID:659.

Another function of VGAM324 is therefore inhibition of LOC257615 (Accession XM_{—}175293). Accordingly, utilities of VGAM324 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 325 (VGAM325) viral gene, which modulates expression of respective host target genes thereof, the function and utility of which host target genes is known in the art.

VGAM325 is a novel bioinformatically detected regulatory, non protein coding, viral micro RNA (miRNA) gene. The method by which VGAM325 was detected is described hereinabove with reference to FIGS. 1-8.

VGAM325 gene, herein designated VGAM GENE, is a viral gene contained in the genome of Vaccinia Virus. VGAM325 host target gene, herein designated VGAM HOST TARGET GENE, is a human gene contained in the human genome.

VGAM325 gene encodes a VGAM325 precursor RNA, herein designated VGAM PRECURSOR RNA. Similar to other miRNA genes, and unlike most ordinary genes, VGAM325 precursor RNA does not encode a protein. A nucleotide sequence identical or highly similar to the nucleotide sequence of VGAM325 precursor RNA is designated SEQ ID:311, and is provided hereinbelow with reference to the sequence listing part. Nucleotide sequence SEQ ID:311 is located at position 188927 relative to the genome of Vaccinia Virus.

VGAM325 precursor RNA folds onto itself, forming VGAM325 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM325 folded precursor RNA into VGAM325 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM325 RNA is designated SEQ ID:660, and is provided hereinbelow with reference to the sequence listing part.

VGAM325 host target gene, herein designated VGAM HOST TARGET GENE, encodes a corresponding messenger RNA, VGAM325 host target RNA, herein designated VGAM HOST TARGET RNA. VGAM325 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM325 RNA, herein designated VGAM RNA, binds complementarily to one or more host target binding sites located in untranslated regions of VGAM325 host target RNA, herein designated VGAM HOST TARGET RNA. This complementary binding is due to the fact that the nucleotide sequence of VGAM325 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM325 RNA, herein designated VGAM RNA, may have a different number of host target binding sites in untranslated regions of a VGAM325 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM325 RNA, herein designated VGAM RNA, to host target binding sites on VGAM325 host target RNA, herein designated VGAM HOST TARGET RNA, such as BINDING SITE I, BINDING SITE II and BINDING SITE III, inhibits translation of VGAM325 host target RNA into VGAM325 host target protein, herein designated VGAM HOST TARGET PROTEIN. VGAM host target protein is therefore outlined by a broken line.

It is appreciated that VGAM325 host target gene, herein designated VGAM HOST TARGET GENE, in fact represents a plurality of VGAM325 host target genes. The mRNA of each one of this plurality of VGAM325 host target genes comprises one or more host target binding sites, each having a nucleotide sequence which is at least partly complementary to VGAM325 RNA, herein designated VGAM RNA, and which when bound by VGAM325 RNA causes inhibition of translation of respective one or more VGAM325 host target proteins.

It is further appreciated by one skilled in the art that the mode of translational inhibition illustrated by FIG. 1 with specific reference to translational inhibition exerted by VGAM325 gene, herein designated VGAM GENE, on one or more VGAM325 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM325 is inhibition of expression of host target genes, as part of a novel viral mechanism of attacking a host. Accordingly, utilities of VGAM325 include diagnosis, prevention and treatment of viral infection by Vaccinia Virus. Specific functions, and accordingly utilities, of VGAM325 correlate with, and may be deduced from, the identity of the host target genes which VGAM325 binds and inhibits, and the function of these host target genes, as elaborated hereinbelow.

Nucleotide sequences of the VGAM325 precursor RNA, herein designated VGAM PRECURSOR RNA, and of the ‘diced’ VGAM325 RNA, herein designated VGAM RNA, and a schematic representation of the secondary folding of VGAM325 folded precursor RNA, herein designated VGAM FOLDED PRECURSOR RNA, of VGAM325 are further described hereinbelow with reference to Table 1.

Nucleotide sequences of host target binding sites, such as BINDING SITE-I, BINDING SITE-II and BINDING SITE-III of FIG. 1, found on VGAM325 host target RNA, and schematic representation of the complementarity of each of these host target binding sites to VGAM325 RNA, herein designated VGAM RNA, are described hereinbelow with reference to Table 2.

As mentioned hereinabove with reference to FIG. 1, a function of VGAM325 gene, herein designated VGAM is inhibition of expression of VGAM325 target genes. It is appreciated that specific functions, and accordingly utilities, of VGAM325 correlate with, and may be deduced from, the identity of the target genes which VGAM325 binds and inhibits, and the function of these target genes, as elaborated hereinbelow.

Calpain 2, (m/II) Large Subunit (CAPN2, Accession NM_{—}001748) is a VGAM325 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM325 RNA, herein designated VGAM RNA, also designated SEQ ID:660.

A function of VGAM325 is therefore inhibition of Calpain 2, (m/II) Large Subunit (CAPN2, Accession NM_{—}001748). Accordingly, utilities of VGAM325 include diagnosis, prevention and treatment of diseases and clinical conditions associated with CAPN2. Dishevelled Associated Activator of Morphogenesis 2 (DAAM2, Accession XM_{—}166434) is another VGAM325 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM325 RNA, herein designated VGAM RNA, also designated SEQ ID:660.

Another function of VGAM325 is therefore inhibition of Dishevelled Associated Activator of Morphogenesis 2 (DAAM2, Accession XM_{—}166434), a gene which controls cell polarity and movement during development. Accordingly, utilities of VGAM325 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM325 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM325 RNA, herein designated VGAM RNA, also designated SEQ ID:660.

Another function of VGAM325 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM325 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM325 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM325 RNA, herein designated VGAM RNA, also designated SEQ ID:660.

Another function of VGAM325 is therefore inhibition of KIAA0475 (Accession NM_{—}014864). Accordingly, utilities of VGAM325 include diagnosis, prevention and treatment of diseases and clinical conditions associated with KIAA0475. LOC222671 (Accession XM_{—}167094) is another VGAM325 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM325 RNA, herein designated VGAM RNA, also designated SEQ ID:660.

Another function of VGAM325 is therefore inhibition of LOC222671 (Accession XM_{—}167094). Accordingly, utilities of VGAM325 include diagnosis, prevention and treatment of diseases and clinical conditions associated with LOC222671. LOC254901 (Accession XM_{—}171878) is another VGAM325 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM325 RNA, herein designated VGAM RNA, also designated SEQ ID:660.

Another function of VGAM325 is therefore inhibition of LOC254901 (Accession XM_{—}171878). Accordingly, utilities of VGAM325 include diagnosis, prevention and treatment of diseases and clinical conditions associated with LOC254901. LOC257615 (Accession XM_{—}175293) is another VGAM325 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM325 RNA, herein designated VGAM RNA, also designated SEQ ID:660.

Another function of VGAM325 is therefore inhibition of LOC257615 (Accession XM_{—}175293). Accordingly, utilities of VGAM325 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 326 (VGAM326) viral gene, which modulates expression of respective host target genes thereof, the function and utility of which host target genes is known in the art.

VGAM326 is a novel bioinformatically detected regulatory, non protein coding, viral micro RNA (miRNA) gene. The method by which VGAM326 was detected is described hereinabove with reference to FIGS. 1-8.

VGAM326 gene, herein designated VGAM GENE, is a viral gene contained in the genome of Vaccinia Virus. VGAM326 host target gene, herein designated VGAM HOST TARGET GENE, is a human gene contained in the human genome.

VGAM326 gene encodes a VGAM326 precursor RNA, herein designated VGAM PRECURSOR RNA. Similar to other miRNA genes, and unlike most ordinary genes, VGAM326 precursor RNA does not encode a protein. A nucleotide sequence identical or highly similar to the nucleotide sequence of VGAM326 precursor RNA is designated SEQ ID:312, and is provided hereinbelow with reference to the sequence listing part. Nucleotide sequence SEQ ID:312 is located at position 188927 relative to the genome of Vaccinia Virus.

VGAM326 precursor RNA folds onto itself, forming VGAM326 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM326 folded precursor RNA into VGAM326 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM326 RNA is designated SEQ ID:661, and is provided hereinbelow with reference to the sequence listing part.

VGAM326 host target gene, herein designated VGAM HOST TARGET GENE, encodes a corresponding messenger RNA, VGAM326 host target RNA, herein designated VGAM HOST TARGET RNA. VGAM326 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM326 RNA, herein designated VGAM RNA, binds complementarily to one or more host target binding sites located in untranslated regions of VGAM326 host target RNA, herein designated VGAM HOST TARGET RNA. This complementary binding is due to the fact that the nucleotide sequence of VGAM326 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM326 RNA, herein designated VGAM RNA, may have a different number of host target binding sites in untranslated regions of a VGAM326 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM326 RNA, herein designated VGAM RNA, to host target binding sites on VGAM326 host target RNA, herein designated VGAM HOST TARGET RNA, such as BINDING SITE I, BINDING SITE II and BINDING SITE III, inhibits translation of VGAM326 host target RNA into VGAM326 host target protein, herein designated VGAM HOST TARGET PROTEIN. VGAM host target protein is therefore outlined by a broken line.

It is appreciated that VGAM326 host target gene, herein designated VGAM HOST TARGET GENE, in fact represents a plurality of VGAM326 host target genes. The mRNA of each one of this plurality of VGAM326 host target genes comprises one or more host target binding sites, each having a nucleotide sequence which is at least partly complementary to VGAM326 RNA, herein designated VGAM RNA, and which when bound by VGAM326 RNA causes inhibition of translation of respective one or more VGAM326 host target proteins.

It is further appreciated by one skilled in the art that the mode of translational inhibition illustrated by FIG. 1 with specific reference to translational inhibition exerted by VGAM326 gene, herein designated VGAM GENE, on one or more VGAM326 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM326 is inhibition of expression of host target genes, as part of a novel viral mechanism of attacking a host. Accordingly, utilities of VGAM326 include diagnosis, prevention and treatment of viral infection by Vaccinia Virus. Specific functions, and accordingly utilities, of VGAM326 correlate with, and may be deduced from, the identity of the host target genes which VGAM326 binds and inhibits, and the function of these host target genes, as elaborated hereinbelow.

Nucleotide sequences of the VGAM326 precursor RNA, herein designated VGAM PRECURSOR RNA, and of the ‘diced’ VGAM326 RNA, herein designated VGAM RNA, and a schematic representation of the secondary folding of VGAM326 folded precursor RNA, herein designated VGAM FOLDED PRECURSOR RNA, of VGAM326 are further described hereinbelow with reference to Table 1.

Nucleotide sequences of host target binding sites, such as BINDING SITE-I, BINDING SITE-II and BINDING SITE-III of FIG. 1, found on VGAM326 host target RNA, and schematic representation of the complementarity of each of these host target binding sites to VGAM326 RNA, herein designated VGAM RNA, are described hereinbelow with reference to Table 2.

As mentioned hereinabove with reference to FIG. 1, a function of VGAM326 gene, herein designated VGAM is inhibition of expression of VGAM326 target genes. It is appreciated that specific functions, and accordingly utilities, of VGAM326 correlate with, and may be deduced from, the identity of the target genes which VGAM326 binds and inhibits, and the function of these target genes, as elaborated hereinbelow.

Calpain 2, (m/II) Large Subunit (CAPN2, Accession NM_{—}001748) is a VGAM326 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM326 RNA, herein designated VGAM RNA, also designated SEQ ID:661.

A function of VGAM326 is therefore inhibition of Calpain 2, (m/II) Large Subunit (CAPN2, Accession NM_{—}001748). Accordingly, utilities of VGAM326 include diagnosis, prevention and treatment of diseases and clinical conditions associated with CAPN2. Dishevelled Associated Activator of Morphogenesis 2 (DAAM2, Accession XM_{—}166434) is another VGAM326 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM326 RNA, herein designated VGAM RNA, also designated SEQ ID:661.

Another function of VGAM326 is therefore inhibition of Dishevelled Associated Activator of Morphogenesis 2 (DAAM2, Accession XM_{—}166434), a gene which controls cell polarity and movement during development. Accordingly, utilities of VGAM326 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM326 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM326 RNA, herein designated VGAM RNA, also designated SEQ ID:661.

Another function of VGAM326 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM326 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM326 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM326 RNA, herein designated VGAM RNA, also designated SEQ ID:661.

Another function of VGAM326 is therefore inhibition of KIAA0475 (Accession NM_{—}014864). Accordingly, utilities of VGAM326 include diagnosis, prevention and treatment of diseases and clinical conditions associated with KIAA0475. LOC222671 (Accession XM_{—}167094) is another VGAM326 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM326 RNA, herein designated VGAM RNA, also designated SEQ ID:661.

Another function of VGAM326 is therefore inhibition of LOC222671 (Accession XM_{—}167094). Accordingly, utilities of VGAM326 include diagnosis, prevention and treatment of diseases and clinical conditions associated with LOC222671. LOC254901 (Accession XM_{—}171878) is another VGAM326 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM326 RNA, herein designated VGAM RNA, also designated SEQ ID:661.

Another function of VGAM326 is therefore inhibition of LOC254901 (Accession XM_{—}171878). Accordingly, utilities of VGAM326 include diagnosis, prevention and treatment of diseases and clinical conditions associated with LOC254901. LOC257615 (Accession XM_{—}175293) is another VGAM326 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM326 RNA, herein designated VGAM RNA, also designated SEQ ID:661.

Another function of VGAM326 is therefore inhibition of LOC257615 (Accession XM_{—}175293). Accordingly, utilities of VGAM326 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 327 (VGAM327) viral gene, which modulates expression of respective host target genes thereof, the function and utility of which host target genes is known in the art.

VGAM327 is a novel bioinformatically detected regulatory, non protein coding, viral micro RNA (miRNA) gene. The method by which VGAM327 was detected is described hereinabove with reference to FIGS. 1-8.

VGAM327 gene, herein designated VGAM GENE, is a viral gene contained in the genome of Vaccinia Virus. VGAM327 host target gene, herein designated VGAM HOST TARGET GENE, is a human gene contained in the human genome.

VGAM327 gene encodes a VGAM327 precursor RNA, herein designated VGAM PRECURSOR RNA. Similar to other miRNA genes, and unlike most ordinary genes, VGAM327 precursor RNA does not encode a protein. A nucleotide sequence identical or highly similar to the nucleotide sequence of VGAM327 precursor RNA is designated SEQ ID:313, and is provided hereinbelow with reference to the sequence listing part. Nucleotide sequence SEQ ID:313 is located at position 188927 relative to the genome of Vaccinia Virus.

VGAM327 precursor RNA folds onto itself, forming VGAM327 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM327 folded precursor RNA into VGAM327 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM327 RNA is designated SEQ ID:662, and is provided hereinbelow with reference to the sequence listing part.

VGAM327 host target gene, herein designated VGAM HOST TARGET GENE, encodes a corresponding messenger RNA, VGAM327 host target RNA, herein designated VGAM HOST TARGET RNA. VGAM327 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM327 RNA, herein designated VGAM RNA, binds complementarily to one or more host target binding sites located in untranslated regions of VGAM327 host target RNA, herein designated VGAM HOST TARGET RNA. This complementary binding is due to the fact that the nucleotide sequence of VGAM327 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM327 RNA, herein designated VGAM RNA, may have a different number of host target binding sites in untranslated regions of a VGAM327 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM327 RNA, herein designated VGAM RNA, to host target binding sites on VGAM327 host target RNA, herein designated VGAM HOST TARGET RNA, such as BINDING SITE I, BINDING SITE II and BINDING SITE III, inhibits translation of VGAM327 host target RNA into VGAM327 host target protein, herein designated VGAM HOST TARGET PROTEIN. VGAM host target protein is therefore outlined by a broken line.

It is appreciated that VGAM327 host target gene, herein designated VGAM HOST TARGET GENE, in fact represents a plurality of VGAM327 host target genes. The mRNA of each one of this plurality of VGAM327 host target genes comprises one or more host target binding sites, each having a nucleotide sequence which is at least partly complementary to VGAM327 RNA, herein designated VGAM RNA, and which when bound by VGAM327 RNA causes inhibition of translation of respective one or more VGAM327 host target proteins.

It is further appreciated by one skilled in the art that the mode of translational inhibition illustrated by FIG. 1 with specific reference to translational inhibition exerted by VGAM327 gene, herein designated VGAM GENE, on one or more VGAM327 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM327 is inhibition of expression of host target genes, as part of a novel viral mechanism of attacking a host. Accordingly, utilities of VGAM327 include diagnosis, prevention and treatment of viral infection by Vaccinia Virus. Specific functions, and accordingly utilities, of VGAM327 correlate with, and may be deduced from, the identity of the host target genes which VGAM327 binds and inhibits, and the function of these host target genes, as elaborated hereinbelow.

Nucleotide sequences of the VGAM327 precursor RNA, herein designated VGAM PRECURSOR RNA, and of the ‘diced’ VGAM327 RNA, herein designated VGAM RNA, and a schematic representation of the secondary folding of VGAM327 folded precursor RNA, herein designated VGAM FOLDED PRECURSOR RNA, of VGAM327 are further described hereinbelow with reference to Table 1.

Nucleotide sequences of host target binding sites, such as BINDING SITE-I, BINDING SITE-II and BINDING SITE-III of FIG. 1, found on VGAM327 host target RNA, and schematic representation of the complementarity of each of these host target binding sites to VGAM327 RNA, herein designated VGAM RNA, are described hereinbelow with reference to Table 2.

As mentioned hereinabove with reference to FIG. 1, a function of VGAM327 gene, herein designated VGAM is inhibition of expression of VGAM327 target genes. It is appreciated that specific functions, and accordingly utilities, of VGAM327 correlate with, and may be deduced from, the identity of the target genes which VGAM327 binds and inhibits, and the function of these target genes, as elaborated hereinbelow.

Calpain 2, (m/II) Large Subunit (CAPN2, Accession NM_{—}001748) is a VGAM327 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM327 RNA, herein designated VGAM RNA, also designated SEQ ID:662.

A function of VGAM327 is therefore inhibition of Calpain 2, (m/II) Large Subunit (CAPN2, Accession NM_{—}001748). Accordingly, utilities of VGAM327 include diagnosis, prevention and treatment of diseases and clinical conditions associated with CAPN2. Dishevelled Associated Activator of Morphogenesis 2 (DAAM2, Accession XM_{—}166434) is another VGAM327 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM327 RNA, herein designated VGAM RNA, also designated SEQ ID:662.

Another function of VGAM327 is therefore inhibition of Dishevelled Associated Activator of Morphogenesis 2 (DAAM2, Accession XM_{—}166434), a gene which controls cell polarity and movement during development. Accordingly, utilities of VGAM327 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM327 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM327 RNA, herein designated VGAM RNA, also designated SEQ ID:662.

Another function of VGAM327 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM327 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM327 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM327 RNA, herein designated VGAM RNA, also designated SEQ ID:662.

Another function of VGAM327 is therefore inhibition of KIAA0475 (Accession NM_{—}014864). Accordingly, utilities of VGAM327 include diagnosis, prevention and treatment of diseases and clinical conditions associated with KIAA0475. LOC222671 (Accession XM_{—}167094) is another VGAM327 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM327 RNA, herein designated VGAM RNA, also designated SEQ ID:662.

Another function of VGAM327 is therefore inhibition of LOC222671 (Accession XM_{—}167094). Accordingly, utilities of VGAM327 include diagnosis, prevention and treatment of diseases and clinical conditions associated with LOC222671. LOC254901 (Accession XM_{—}171878) is another VGAM327 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM327 RNA, herein designated VGAM RNA, also designated SEQ ID:662.

Another function of VGAM327 is therefore inhibition of LOC254901 (Accession XM_{—}171878). Accordingly, utilities of VGAM327 include diagnosis, prevention and treatment of diseases and clinical conditions associated with LOC254901. LOC257615 (Accession XM_{—}175293) is another VGAM327 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM327 RNA, herein designated VGAM RNA, also designated SEQ ID:662.

Another function of VGAM327 is therefore inhibition of LOC257615 (Accession XM_{—}175293). Accordingly, utilities of VGAM327 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 328 (VGAM328) viral gene, which modulates expression of respective host target genes thereof, the function and utility of which host target genes is known in the art.

VGAM328 is a novel bioinformatically detected regulatory, non protein coding, viral micro RNA (miRNA) gene. The method by which VGAM328 was detected is described hereinabove with reference to FIGS. 1-8.

VGAM328 gene, herein designated VGAM GENE, is a viral gene contained in the genome of Vaccinia Virus. VGAM328 host target gene, herein designated VGAM HOST TARGET GENE, is a human gene contained in the human genome.

VGAM328 gene encodes a VGAM328 precursor RNA, herein designated VGAM PRECURSOR RNA. Similar to other miRNA genes, and unlike most ordinary genes, VGAM328 precursor RNA does not encode a protein. A nucleotide sequence identical or highly similar to the nucleotide sequence of VGAM328 precursor RNA is designated SEQ ID:314, and is provided hereinbelow with reference to the sequence listing part. Nucleotide sequence SEQ ID:314 is located at position 188927 relative to the genome of Vaccinia Virus.

VGAM328 precursor RNA folds onto itself, forming VGAM328 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM328 folded precursor RNA into VGAM328 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM328 RNA is designated SEQ ID:663, and is provided hereinbelow with reference to the sequence listing part.

VGAM328 host target gene, herein designated VGAM HOST TARGET GENE, encodes a corresponding messenger RNA, VGAM328 host target RNA, herein designated VGAM HOST TARGET RNA. VGAM328 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM328 RNA, herein designated VGAM RNA, binds complementarily to one or more host target binding sites located in untranslated regions of VGAM328 host target RNA, herein designated VGAM HOST TARGET RNA. This complementary binding is due to the fact that the nucleotide sequence of VGAM328 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM328 RNA, herein designated VGAM RNA, may have a different number of host target binding sites in untranslated regions of a VGAM328 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM328 RNA, herein designated VGAM RNA, to host target binding sites on VGAM328 host target RNA, herein designated VGAM HOST TARGET RNA, such as BINDING SITE I, BINDING SITE II and BINDING SITE III, inhibits translation of VGAM328 host target RNA into VGAM328 host target protein, herein designated VGAM HOST TARGET PROTEIN. VGAM host target protein is therefore outlined by a broken line.

It is appreciated that VGAM328 host target gene, herein designated VGAM HOST TARGET GENE, in fact represents a plurality of VGAM328 host target genes. The mRNA of each one of this plurality of VGAM328 host target genes comprises one or more host target binding sites, each having a nucleotide sequence which is at least partly complementary to VGAM328 RNA, herein designated VGAM RNA, and which when bound by VGAM328 RNA causes inhibition of translation of respective one or more VGAM328 host target proteins.

It is further appreciated by one skilled in the art that the mode of translational inhibition illustrated by FIG. 1 with specific reference to translational inhibition exerted by VGAM328 gene, herein designated VGAM GENE, on one or more VGAM328 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM328 is inhibition of expression of host target genes, as part of a novel viral mechanism of attacking a host. Accordingly, utilities of VGAM328 include diagnosis, prevention and treatment of viral infection by Vaccinia Virus. Specific functions, and accordingly utilities, of VGAM328 correlate with, and may be deduced from, the identity of the host target genes which VGAM328 binds and inhibits, and the function of these host target genes, as elaborated hereinbelow.

Nucleotide sequences of the VGAM328 precursor RNA, herein designated VGAM PRECURSOR RNA, and of the ‘diced’ VGAM328 RNA, herein designated VGAM RNA, and a schematic representation of the secondary folding of VGAM328 folded precursor RNA, herein designated VGAM FOLDED PRECURSOR RNA, of VGAM328 are further described hereinbelow with reference to Table 1.

Nucleotide sequences of host target binding sites, such as BINDING SITE-I, BINDING SITE-II and BINDING SITE-III of FIG. 1, found on VGAM328 host target RNA, and schematic representation of the complementarity of each of these host target binding sites to VGAM328 RNA, herein designated VGAM RNA, are described hereinbelow with reference to Table 2.

As mentioned hereinabove with reference to FIG. 1, a function of VGAM328 gene, herein designated VGAM is inhibition of expression of VGAM328 target genes. It is appreciated that specific functions, and accordingly utilities, of VGAM328 correlate with, and may be deduced from, the identity of the target genes which VGAM328 binds and inhibits, and the function of these target genes, as elaborated hereinbelow.

Calpain 2, (m/II) Large Subunit (CAPN2, Accession NM_{—}001748) is a VGAM328 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM328 RNA, herein designated VGAM RNA, also designated SEQ ID:663.

A function of VGAM328 is therefore inhibition of Calpain 2, (m/II) Large Subunit (CAPN2, Accession NM_{—}001748). Accordingly, utilities of VGAM328 include diagnosis, prevention and treatment of diseases and clinical conditions associated with CAPN2. Dishevelled Associated Activator of Morphogenesis 2 (DAAM2, Accession XM_{—}166434) is another VGAM328 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM328 RNA, herein designated VGAM RNA, also designated SEQ ID:663.

Another function of VGAM328 is therefore inhibition of Dishevelled Associated Activator of Morphogenesis 2 (DAAM2, Accession XM_{—}166434), a gene which controls cell polarity and movement during development. Accordingly, utilities of VGAM328 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM328 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM328 RNA, herein designated VGAM RNA, also designated SEQ ID:663.

Another function of VGAM328 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM328 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM328 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM328 RNA, herein designated VGAM RNA, also designated SEQ ID:663.

Another function of VGAM328 is therefore inhibition of KIAA0475 (Accession NM_{—}014864). Accordingly, utilities of VGAM328 include diagnosis, prevention and treatment of diseases and clinical conditions associated with KIAA0475. LOC222671 (Accession XM_{—}167094) is another VGAM328 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM328 RNA, herein designated VGAM RNA, also designated SEQ ID:663.

Another function of VGAM328 is therefore inhibition of LOC222671 (Accession XM_{—}167094). Accordingly, utilities of VGAM328 include diagnosis, prevention and treatment of diseases and clinical conditions associated with LOC222671. LOC254901 (Accession XM_{—}171878) is another VGAM328 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM328 RNA, herein designated VGAM RNA, also designated SEQ ID:663.

Another function of VGAM328 is therefore inhibition of LOC254901 (Accession XM_{—}171878). Accordingly, utilities of VGAM328 include diagnosis, prevention and treatment of diseases and clinical conditions associated with LOC254901. LOC257615 (Accession XM_{—}175293) is another VGAM328 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM328 RNA, herein designated VGAM RNA, also designated SEQ ID:663.

Another function of VGAM328 is therefore inhibition of LOC257615 (Accession XM_{—}175293). Accordingly, utilities of VGAM328 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 329 (VGAM329) viral gene, which modulates expression of respective host target genes thereof, the function and utility of which host target genes is known in the art.

VGAM329 is a novel bioinformatically detected regulatory, non protein coding, viral micro RNA (miRNA) gene. The method by which VGAM329 was detected is described hereinabove with reference to FIGS. 1-8.

VGAM329 gene, herein designated VGAM GENE, is a viral gene contained in the genome of Vaccinia Virus. VGAM329 host target gene, herein designated VGAM HOST TARGET GENE, is a human gene contained in the human genome.

VGAM329 gene encodes a VGAM329 precursor RNA, herein designated VGAM PRECURSOR RNA. Similar to other miRNA genes, and unlike most ordinary genes, VGAM329 precursor RNA does not encode a protein. A nucleotide sequence identical or highly similar to the nucleotide sequence of VGAM329 precursor RNA is designated SEQ ID:315, and is provided hereinbelow with reference to the sequence listing part. Nucleotide sequence SEQ ID:315 is located at position 188927 relative to the genome of Vaccinia Virus.

VGAM329 precursor RNA folds onto itself, forming VGAM329 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM329 folded precursor RNA into VGAM329 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM329 RNA is designated SEQ ID:664, and is provided hereinbelow with reference to the sequence listing part.

VGAM329 host target gene, herein designated VGAM HOST TARGET GENE, encodes a corresponding messenger RNA, VGAM329 host target RNA, herein designated VGAM HOST TARGET RNA. VGAM329 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM329 RNA, herein designated VGAM RNA, binds complementarily to one or more host target binding sites located in untranslated regions of VGAM329 host target RNA, herein designated VGAM HOST TARGET RNA. This complementary binding is due to the fact that the nucleotide sequence of VGAM329 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM329 RNA, herein designated VGAM RNA, may have a different number of host target binding sites in untranslated regions of a VGAM329 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM329 RNA, herein designated VGAM RNA, to host target binding sites on VGAM329 host target RNA, herein designated VGAM HOST TARGET RNA, such as BINDING SITE I, BINDING SITE II and BINDING SITE III, inhibits translation of VGAM329 host target RNA into VGAM329 host target protein, herein designated VGAM HOST TARGET PROTEIN. VGAM host target protein is therefore outlined by a broken line.

It is appreciated that VGAM329 host target gene, herein designated VGAM HOST TARGET GENE, in fact represents a plurality of VGAM329 host target genes. The mRNA of each one of this plurality of VGAM329 host target genes comprises one or more host target binding sites, each having a nucleotide sequence which is at least partly complementary to VGAM329 RNA, herein designated VGAM RNA, and which when bound by VGAM329 RNA causes inhibition of translation of respective one or more VGAM329 host target proteins.

It is further appreciated by one skilled in the art that the mode of translational inhibition illustrated by FIG. 1 with specific reference to translational inhibition exerted by VGAM329 gene, herein designated VGAM GENE, on one or more VGAM329 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM329 is inhibition of expression of host target genes, as part of a novel viral mechanism of attacking a host. Accordingly, utilities of VGAM329 include diagnosis, prevention and treatment of viral infection by Vaccinia Virus. Specific functions, and accordingly utilities, of VGAM329 correlate with, and may be deduced from, the identity of the host target genes which VGAM329 binds and inhibits, and the function of these host target genes, as elaborated hereinbelow.

Nucleotide sequences of the VGAM329 precursor RNA, herein designated VGAM PRECURSOR RNA, and of the ‘diced’ VGAM329 RNA, herein designated VGAM RNA, and a schematic representation of the secondary folding of VGAM329 folded precursor RNA, herein designated VGAM FOLDED PRECURSOR RNA, of VGAM329 are further described hereinbelow with reference to Table 1.

Nucleotide sequences of host target binding sites, such as BINDING SITE-I, BINDING SITE-II and BINDING SITE-III of FIG. 1, found on VGAM329 host target RNA, and schematic representation of the complementarity of each of these host target binding sites to VGAM329 RNA, herein designated VGAM RNA, are described hereinbelow with reference to Table 2.

As mentioned hereinabove with reference to FIG. 1, a function of VGAM329 gene, herein designated VGAM is inhibition of expression of VGAM329 target genes. It is appreciated that specific functions, and accordingly utilities, of VGAM329 correlate with, and may be deduced from, the identity of the target genes which VGAM329 binds and inhibits, and the function of these target genes, as elaborated hereinbelow.

Calpain 2, (m/II) Large Subunit (CAPN2, Accession NM_{—}001748) is a VGAM329 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM329 RNA, herein designated VGAM RNA, also designated SEQ ID:664.

A function of VGAM329 is therefore inhibition of Calpain 2, (m/II) Large Subunit (CAPN2, Accession NM_{—}001748). Accordingly, utilities of VGAM329 include diagnosis, prevention and treatment of diseases and clinical conditions associated with CAPN2. Dishevelled Associated Activator of Morphogenesis 2 (DAAM2, Accession XM_{—}166434) is another VGAM329 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM329 RNA, herein designated VGAM RNA, also designated SEQ ID:664.

Another function of VGAM329 is therefore inhibition of Dishevelled Associated Activator of Morphogenesis 2 (DAAM2, Accession XM_{—}166434), a gene which controls cell polarity and movement during development. Accordingly, utilities of VGAM329 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM329 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM329 RNA, herein designated VGAM RNA, also designated SEQ ID:664.

Another function of VGAM329 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM329 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM329 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM329 RNA, herein designated VGAM RNA, also designated SEQ ID:664.

Another function of VGAM329 is therefore inhibition of KIAA0475 (Accession NM_{—}014864). Accordingly, utilities of VGAM329 include diagnosis, prevention and treatment of diseases and clinical conditions associated with KIAA0475. LOC222671 (Accession XM_{—}167094) is another VGAM329 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM329 RNA, herein designated VGAM RNA, also designated SEQ ID:664.

Another function of VGAM329 is therefore inhibition of LOC222671 (Accession XM_{—}167094). Accordingly, utilities of VGAM329 include diagnosis, prevention and treatment of diseases and clinical conditions associated with LOC222671. LOC254901 (Accession XM_{—}171878) is another VGAM329 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM329 RNA, herein designated VGAM RNA, also designated SEQ ID:664.

Another function of VGAM329 is therefore inhibition of LOC254901 (Accession XM_{—}171878). Accordingly, utilities of VGAM329 include diagnosis, prevention and treatment of diseases and clinical conditions associated with LOC254901. LOC257615 (Accession XM_{—}175293) is another VGAM329 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM329 RNA, herein designated VGAM RNA, also designated SEQ ID:664.

Another function of VGAM329 is therefore inhibition of LOC257615 (Accession XM_{—}175293). Accordingly, utilities of VGAM329 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 330 (VGAM330) viral gene, which modulates expression of respective host target genes thereof, the function and utility of which host target genes is known in the art.

VGAM330 is a novel bioinformatically detected regulatory, non protein coding, viral micro RNA (miRNA) gene. The method by which VGAM330 was detected is described hereinabove with reference to FIGS. 1-8.

VGAM330 gene, herein designated VGAM GENE, is a viral gene contained in the genome of Vaccinia Virus. VGAM330 host target gene, herein designated VGAM HOST TARGET GENE, is a human gene contained in the human genome.

VGAM330 gene encodes a VGAM330 precursor RNA, herein designated VGAM PRECURSOR RNA. Similar to other miRNA genes, and unlike most ordinary genes, VGAM330 precursor RNA does not encode a protein. A nucleotide sequence identical or highly similar to the nucleotide sequence of VGAM330 precursor RNA is designated SEQ ID:316, and is provided hereinbelow with reference to the sequence listing part. Nucleotide sequence SEQ ID:316 is located at position 188927 relative to the genome of Vaccinia Virus.

VGAM330 precursor RNA folds onto itself, forming VGAM330 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM330 folded precursor RNA into VGAM330 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM330 RNA is designated SEQ ID:665, and is provided hereinbelow with reference to the sequence listing part.

VGAM330 host target gene, herein designated VGAM HOST TARGET GENE, encodes a corresponding messenger RNA, VGAM330 host target RNA, herein designated VGAM HOST TARGET RNA. VGAM330 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM330 RNA, herein designated VGAM RNA, binds complementarily to one or more host target binding sites located in untranslated regions of VGAM330 host target RNA, herein designated VGAM HOST TARGET RNA. This complementary binding is due to the fact that the nucleotide sequence of VGAM330 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM330 RNA, herein designated VGAM RNA, may have a different number of host target binding sites in untranslated regions of a VGAM330 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM330 RNA, herein designated VGAM RNA, to host target binding sites on VGAM330 host target RNA, herein designated VGAM HOST TARGET RNA, such as BINDING SITE I, BINDING SITE II and BINDING SITE III, inhibits translation of VGAM330 host target RNA into VGAM330 host target protein, herein designated VGAM HOST TARGET PROTEIN. VGAM host target protein is therefore outlined by a broken line.

It is appreciated that VGAM330 host target gene, herein designated VGAM HOST TARGET GENE, in fact represents a plurality of VGAM330 host target genes. The mRNA of each one of this plurality of VGAM330 host target genes comprises one or more host target binding sites, each having a nucleotide sequence which is at least partly complementary to VGAM330 RNA, herein designated VGAM RNA, and which when bound by VGAM330 RNA causes inhibition of translation of respective one or more VGAM330 host target proteins.

It is further appreciated by one skilled in the art that the mode of translational inhibition illustrated by FIG. 1 with specific reference to translational inhibition exerted by VGAM330 gene, herein designated VGAM GENE, on one or more VGAM330 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM330 is inhibition of expression of host target genes, as part of a novel viral mechanism of attacking a host. Accordingly, utilities of VGAM330 include diagnosis, prevention and treatment of viral infection by Vaccinia Virus. Specific functions, and accordingly utilities, of VGAM330 correlate with, and may be deduced from, the identity of the host target genes which VGAM330 binds and inhibits, and the function of these host target genes, as elaborated hereinbelow.

Nucleotide sequences of the VGAM330 precursor RNA, herein designated VGAM PRECURSOR RNA, and of the ‘diced’ VGAM330 RNA, herein designated VGAM RNA, and a schematic representation of the secondary folding of VGAM330 folded precursor RNA, herein designated VGAM FOLDED PRECURSOR RNA, of VGAM330 are further described hereinbelow with reference to Table 1.

Nucleotide sequences of host target binding sites, such as BINDING SITE-I, BINDING SITE-II and BINDING SITE-III of FIG. 1, found on VGAM330 host target RNA, and schematic representation of the complementarity of each of these host target binding sites to VGAM330 RNA, herein designated VGAM RNA, are described hereinbelow with reference to Table 2.

As mentioned hereinabove with reference to FIG. 1, a function of VGAM330 gene, herein designated VGAM is inhibition of expression of VGAM330 target genes. It is appreciated that specific functions, and accordingly utilities, of VGAM330 correlate with, and may be deduced from, the identity of the target genes which VGAM330 binds and inhibits, and the function of these target genes, as elaborated hereinbelow.

Calpain 2, (m/II) Large Subunit (CAPN2, Accession NM_{—}001748) is a VGAM330 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM330 RNA, herein designated VGAM RNA, also designated SEQ ID:665.

A function of VGAM330 is therefore inhibition of Calpain 2, (m/II) Large Subunit (CAPN2, Accession NM_{—}001748). Accordingly, utilities of VGAM330 include diagnosis, prevention and treatment of diseases and clinical conditions associated with CAPN2. Dishevelled Associated Activator of Morphogenesis 2 (DAAM2, Accession XM_{—}166434) is another VGAM330 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM330 RNA, herein designated VGAM RNA, also designated SEQ ID:665.

Another function of VGAM330 is therefore inhibition of Dishevelled Associated Activator of Morphogenesis 2 (DAAM2, Accession XM_{—}166434), a gene which controls cell polarity and movement during development. Accordingly, utilities of VGAM330 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM330 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM330 RNA, herein designated VGAM RNA, also designated SEQ ID:665.

Another function of VGAM330 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM330 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM330 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM330 RNA, herein designated VGAM RNA, also designated SEQ ID:665.

Another function of VGAM330 is therefore inhibition of KIAA0475 (Accession NM_{—}014864). Accordingly, utilities of VGAM330 include diagnosis, prevention and treatment of diseases and clinical conditions associated with KIAA0475. LOC222671 (Accession XM_{—}167094) is another VGAM330 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM330 RNA, herein designated VGAM RNA, also designated SEQ ID:665.

Another function of VGAM330 is therefore inhibition of LOC222671 (Accession XM_{—}167094). Accordingly, utilities of VGAM330 include diagnosis, prevention and treatment of diseases and clinical conditions associated with LOC222671. LOC254901 (Accession XM_{—}171878) is another VGAM330 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM330 RNA, herein designated VGAM RNA, also designated SEQ ID:665.

Another function of VGAM330 is therefore inhibition of LOC254901 (Accession XM_{—}171878). Accordingly, utilities of VGAM330 include diagnosis, prevention and treatment of diseases and clinical conditions associated with LOC254901. LOC257615 (Accession XM_{—}175293) is another VGAM330 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM330 RNA, herein designated VGAM RNA, also designated SEQ ID:665.

Another function of VGAM330 is therefore inhibition of LOC257615 (Accession XM_{—}175293). Accordingly, utilities of VGAM330 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 331 (VGAM331) viral gene, which modulates expression of respective host target genes thereof, the function and utility of which host target genes is known in the art.

VGAM331 is a novel bioinformatically detected regulatory, non protein coding, viral micro RNA (miRNA) gene. The method by which VGAM331 was detected is described hereinabove with reference to FIGS. 1-8.

VGAM331 gene, herein designated VGAM GENE, is a viral gene contained in the genome of Vaccinia Virus. VGAM331 host target gene, herein designated VGAM HOST TARGET GENE, is a human gene contained in the human genome.

VGAM331 gene encodes a VGAM331 precursor RNA, herein designated VGAM PRECURSOR RNA. Similar to other miRNA genes, and unlike most ordinary genes, VGAM331 precursor RNA does not encode a protein. A nucleotide sequence identical or highly similar to the nucleotide sequence of VGAM331 precursor RNA is designated SEQ ID:317, and is provided hereinbelow with reference to the sequence listing part. Nucleotide sequence SEQ ID:317 is located at position 189067 relative to the genome of Vaccinia Virus.

VGAM331 precursor RNA folds onto itself, forming VGAM331 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM331 folded precursor RNA into VGAM331 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 78%) nucleotide sequence of VGAM331 RNA is designated SEQ ID:666, and is provided hereinbelow with reference to the sequence listing part.

VGAM331 host target gene, herein designated VGAM HOST TARGET GENE, encodes a corresponding messenger RNA, VGAM331 host target RNA, herein designated VGAM HOST TARGET RNA. VGAM331 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM331 RNA, herein designated VGAM RNA, binds complementarily to one or more host target binding sites located in untranslated regions of VGAM331 host target RNA, herein designated VGAM HOST TARGET RNA. This complementary binding is due to the fact that the nucleotide sequence of VGAM331 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM331 RNA, herein designated VGAM RNA, may have a different number of host target binding sites in untranslated regions of a VGAM331 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM331 RNA, herein designated VGAM RNA, to host target binding sites on VGAM331 host target RNA, herein designated VGAM HOST TARGET RNA, such as BINDING SITE I, BINDING SITE II and BINDING SITE III, inhibits translation of VGAM331 host target RNA into VGAM331 host target protein, herein designated VGAM HOST TARGET PROTEIN. VGAM host target protein is therefore outlined by a broken line.

It is appreciated that VGAM331 host target gene, herein designated VGAM HOST TARGET GENE, in fact represents a plurality of VGAM331 host target genes. The mRNA of each one of this plurality of VGAM331 host target genes comprises one or more host target binding sites, each having a nucleotide sequence which is at least partly complementary to VGAM331 RNA, herein designated VGAM RNA, and which when bound by VGAM331 RNA causes inhibition of translation of respective one or more VGAM331 host target proteins.

It is further appreciated by one skilled in the art that the mode of translational inhibition illustrated by FIG. 1 with specific reference to translational inhibition exerted by VGAM331 gene, herein designated VGAM GENE, on one or more VGAM331 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM331 is inhibition of expression of host target genes, as part of a novel viral mechanism of attacking a host. Accordingly, utilities of VGAM331 include diagnosis, prevention and treatment of viral infection by Vaccinia Virus. Specific functions, and accordingly utilities, of VGAM331 correlate with, and may be deduced from, the identity of the host target genes which VGAM331 binds and inhibits, and the function of these host target genes, as elaborated hereinbelow.

Nucleotide sequences of the VGAM331 precursor RNA, herein designated VGAM PRECURSOR RNA, and of the ‘diced’ VGAM331 RNA, herein designated VGAM RNA, and a schematic representation of the secondary folding of VGAM331 folded precursor RNA, herein designated VGAM FOLDED PRECURSOR RNA, of VGAM331 are further described hereinbelow with reference to Table 1.

Nucleotide sequences of host target binding sites, such as BINDING SITE-I, BINDING SITE-II and BINDING SITE-III of FIG. 1, found on VGAM331 host target RNA, and schematic representation of the complementarity of each of these host target binding sites to VGAM331 RNA, herein designated VGAM RNA, are described hereinbelow with reference to Table 2.

As mentioned hereinabove with reference to FIG. 1, a function of VGAM331 gene, herein designated VGAM is inhibition of expression of VGAM331 target genes. It is appreciated that specific functions, and accordingly utilities, of VGAM331 correlate with, and may be deduced from, the identity of the target genes which VGAM331 binds and inhibits, and the function of these target genes, as elaborated hereinbelow.

Integrin, Beta 7 (ITGB7, Accession NM_{—}000889) is a VGAM331 host target gene. ITGB7 BINDING SITE is HOST TARGET binding site found in the 5′ untranslated region of mRNA encoded by ITGB7, corresponding to a HOST TARGET binding site such as BINDING SITE I, BINDING SITE II or BINDING SITE III. Table 2 illustrates the complementarity of the nucleotide sequences of ITGB7 BINDING SITE, designated SEQ ID:783, to the nucleotide sequence of VGAM331 RNA, herein designated VGAM RNA, also designated SEQ ID:666.

A function of VGAM331 is therefore inhibition of Integrin, Beta 7 (ITGB7, Accession NM_{—}000889), a gene which may play a role in adhesive interactions of leukocytes. Accordingly, utilities of VGAM331 include diagnosis, prevention and treatment of diseases and clinical conditions associated with ITGB7. The function of ITGB7 and its association with various diseases and clinical conditions, has been established by previous studies, as described hereinabove with reference to VGAM26. RPP30 (Accession NM_{—}006413) is another VGAM331 host target gene. RPP30 BINDING SITE is HOST TARGET binding site found in the 3′ untranslated region of mRNA encoded by RPP30, corresponding to a HOST TARGET binding site such as BINDING SITE I, BINDING SITE II or BINDING SITE III. Table 2 illustrates the complementarity of the nucleotide sequences of RPP30 BINDING SITE, designated SEQ ID:1297, to the nucleotide sequence of VGAM331 RNA, herein designated VGAM RNA, also designated SEQ ID:666.

Another function of VGAM331 is therefore inhibition of RPP30 (Accession NM_{—}006413), a gene which is a component of ribonuclease p that processes 5′ ends of precursor tRNAs. Accordingly, utilities of VGAM331 include diagnosis, prevention and treatment of diseases and clinical conditions associated with RPP30. The function of RPP30 and its association with various diseases and clinical conditions, has been established by previous studies, as described hereinabove with reference to VGAM26. FLJ13263 (Accession NM_{—}025125) is another VGAM331 host target gene. FLJ13263 BINDING SITE is HOST TARGET binding site found in the 3′ untranslated region of mRNA encoded by FLJ13263, corresponding to a HOST TARGET binding site such as BINDING SITE I, BINDING SITE II or BINDING SITE III. Table 2 illustrates the complementarity of the nucleotide sequences of FLJ13263 BINDING SITE, designated SEQ ID:2139, to the nucleotide sequence of VGAM331 RNA, herein designated VGAM RNA, also designated SEQ ID:666.

Another function of VGAM331 is therefore inhibition of FLJ13263 (Accession NM_{—}025125). Accordingly, utilities of VGAM331 include diagnosis, prevention and treatment of diseases and clinical conditions associated with FLJ13263. FLJ23074 (Accession NM_{—}025052) is another VGAM331 host target gene. FLJ23074 BINDING SITE is HOST TARGET binding site found in the 5′ untranslated region of mRNA encoded by FLJ23074, corresponding to a HOST TARGET binding site such as BINDING SITE I, BINDING SITE II or BINDING SITE III. Table 2 illustrates the complementarity of the nucleotide sequences of FLJ23074 BINDING SITE, designated SEQ ID:2133, to the nucleotide sequence of VGAM331 RNA, herein designated VGAM RNA, also designated SEQ ID:666.

Another function of VGAM331 is therefore inhibition of FLJ23074 (Accession NM_{—}025052). Accordingly, utilities of VGAM331 include diagnosis, prevention and treatment of diseases and clinical conditions associated with FLJ23074. General Transcription Factor IIA, 2, 12 kDa (GTF2A2, Accession NM_{—}004492) is another VGAM331 host target gene. GTF2A2 BINDING SITE is HOST TARGET binding site found in the 3′ untranslated region of mRNA encoded by GTF2A2, corresponding to a HOST TARGET binding site such as BINDING SITE I, BINDING SITE II or BINDING SITE III. Table 2 illustrates the complementarity of the nucleotide sequences of GTF2A2 BINDING SITE, designated SEQ ID:1119, to the nucleotide sequence of VGAM331 RNA, herein designated VGAM RNA, also designated SEQ ID:666.

Another function of VGAM331 is therefore inhibition of General Transcription Factor IIA, 2, 12 kDa (GTF2A2, Accession NM_{—}004492). Accordingly, utilities of VGAM331 include diagnosis, prevention and treatment of diseases and clinical conditions associated with GTF2A2. IMP-2 (Accession NM_{—}006548) is another VGAM331 host target gene. IMP-2 BINDING SITE is HOST TARGET binding site found in the 3′ untranslated region of mRNA encoded by IMP-2, corresponding to a HOST TARGET binding site such as BINDING SITE I, BINDING SITE II or BINDING SITE III. Table 2 illustrates the complementarity of the nucleotide sequences of IMP-2 BINDING SITE, designated SEQ ID:1308, to the nucleotide sequence of VGAM331 RNA, herein designated VGAM RNA, also designated SEQ ID:666.

Another function of VGAM331 is therefore inhibition of IMP-2 (Accession NM_{—}006548). Accordingly, utilities of VGAM331 include diagnosis, prevention and treatment of diseases and clinical conditions associated with IMP-2. KIAA0212 (Accession NM_{—}014674) is another VGAM331 host target gene. KIAA0212 BINDING SITE is HOST TARGET binding site found in the 3′ untranslated region of mRNA encoded by KIAA0212, corresponding to a HOST TARGET binding site such as BINDING SITE I, BINDING SITE II or BINDING SITE III. Table 2 illustrates the complementarity of the nucleotide sequences of KIAA0212 BINDING SITE, designated SEQ ID:1518, to the nucleotide sequence of VGAM331 RNA, herein designated VGAM RNA, also designated SEQ ID:666.

Another function of VGAM331 is therefore inhibition of KIAA0212 (Accession NM_{—}014674). Accordingly, utilities of VGAM331 include diagnosis, prevention and treatment of diseases and clinical conditions associated with KIAA0212. Oxysterol Binding Protein-like 2 (OSBPL2, Accession NM_{—}014835) is another VGAM331 host target gene. OSBPL2 BINDING SITE1 and OSBPL2 BINDING SITE2 are HOST TARGET binding sites found in untranslated regions of mRNA encoded by OSBPL2, corresponding to HOST TARGET binding sites such as BINDING SITE I, BINDING SITE II or BINDING SITE III. Table 2 illustrates the complementarity of the nucleotide sequences of OSBPL2 BINDING SITE1 and OSBPL2 BINDING SITE2, designated SEQ ID: 1566 and SEQ ID:2482 respectively, to the nucleotide sequence of VGAM331 RNA, herein designated VGAM RNA, also designated SEQ ID:666.

Another function of VGAM331 is therefore inhibition of Oxysterol Binding Protein-like 2 (OSBPL2, Accession NM_{—}014835). Accordingly, utilities of VGAM331 include diagnosis, prevention and treatment of diseases and clinical conditions associated with OSBPL2. LOC254826 (Accession XM_{—}173188) is another VGAM331 host target gene. LOC254826 BINDING SITE is HOST TARGET binding site found in the 3′ untranslated region of mRNA encoded by LOC254826, corresponding to a HOST TARGET binding site such as BINDING SITE I, BINDING SITE II or BINDING SITE III. Table 2 illustrates the complementarity of the nucleotide sequences of LOC254826 BINDING SITE, designated SEQ ID:3732, to the nucleotide sequence of VGAM331 RNA, herein designated VGAM RNA, also designated SEQ ID:666.

Another function of VGAM331 is therefore inhibition of LOC254826 (Accession XM_{—}173188). Accordingly, utilities of VGAM331 include diagnosis, prevention and treatment of diseases and clinical conditions associated with LOC254826. FIG. 1 further provides a conceptual description of a novel bioinformatically detected viral gene of the present invention, referred to here as Viral Genomic Address Messenger 332 (VGAM332) viral gene, which modulates expression of respective host target genes thereof, the function and utility of which host target genes is known in the art.

VGAM332 is a novel bioinformatically detected regulatory, non protein coding, viral micro RNA (miRNA) gene. The method by which VGAM332 was detected is described hereinabove with reference to FIGS. 1-8.

VGAM332 gene, herein designated VGAM GENE, is a viral gene contained in the genome of Vaccinia Virus. VGAM332 host target gene, herein designated VGAM HOST TARGET GENE, is a human gene contained in the human genome.

VGAM332 gene encodes a VGAM332 precursor RNA, herein designated VGAM PRECURSOR RNA. Similar to other miRNA genes, and unlike most ordinary genes, VGAM332 precursor RNA does not encode a protein. A nucleotide sequence identical or highly similar to the nucleotide sequence of VGAM332 precursor RNA is designated SEQ ID:318, and is provided hereinbelow with reference to the sequence listing part. Nucleotide sequence SEQ ID:318 is located at position 11 relative to the genome of Vaccinia Virus.

VGAM332 precursor RNA folds onto itself, forming VGAM332 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM332 folded precursor RNA into VGAM332 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM332 RNA is designated SEQ ID:667, and is provided hereinbelow with reference to the sequence listing part.

VGAM332 host target gene, herein designated VGAM HOST TARGET GENE, encodes a corresponding messenger RNA, VGAM332 host target RNA, herein designated VGAM HOST TARGET RNA. VGAM332 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM332 RNA, herein designated VGAM RNA, binds complementarily to one or more host target binding sites located in untranslated regions of VGAM332 host target RNA, herein designated VGAM HOST TARGET RNA. This complementary binding is due to the fact that the nucleotide sequence of VGAM332 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM332 RNA, herein designated VGAM RNA, may have a different number of host target binding sites in untranslated regions of a VGAM332 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM332 RNA, herein designated VGAM RNA, to host target binding sites on VGAM332 host target RNA, herein designated VGAM HOST TARGET RNA, such as BINDING SITE I, BINDING SITE II and BINDING SITE III, inhibits translation of VGAM332 host target RNA into VGAM332 host target protein, herein designated VGAM HOST TARGET PROTEIN. VGAM host target protein is therefore outlined by a broken line.

It is appreciated that VGAM332 host target gene, herein designated VGAM HOST TARGET GENE, in fact represents a plurality of VGAM332 host target genes. The mRNA of each one of this plurality of VGAM332 host target genes comprises one or more host target binding sites, each having a nucleotide sequence which is at least partly complementary to VGAM332 RNA, herein designated VGAM RNA, and which when bound by VGAM332 RNA causes inhibition of translation of respective one or more VGAM332 host target proteins.

It is further appreciated by one skilled in the art that the mode of translational inhibition illustrated by FIG. 1 with specific reference to translational inhibition exerted by VGAM332 gene, herein designated VGAM GENE, on one or more VGAM332 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM332 is inhibition of expression of host target genes, as part of a novel viral mechanism of attacking a host. Accordingly, utilities of VGAM332 include diagnosis, prevention and treatment of viral infection by Vaccinia Virus. Specific functions, and accordingly utilities, of VGAM332 correlate with, and may be deduced from, the identity of the host target genes which VGAM332 binds and inhibits, and the function of these host target genes, as elaborated hereinbelow.

Nucleotide sequences of the VGAM332 precursor RNA, herein designated VGAM PRECURSOR RNA, and of the ‘diced’ VGAM332 RNA, herein designated VGAM RNA, and a schematic representation of the secondary folding of VGAM332 folded precursor RNA, herein designated VGAM FOLDED PRECURSOR RNA, of VGAM332 are further described hereinbelow with reference to Table 1.

Nucleotide sequences of host target binding sites, such as BINDING SITE-I, BINDING SITE-II and BINDING SITE-III of FIG. 1, found on VGAM332 host target RNA, and schematic representation of the complementarity of each of these host target binding sites to VGAM332 RNA, herein designated VGAM RNA, are described hereinbelow with reference to Table 2.

As mentioned hereinabove with reference to FIG. 1, a function of VGAM332 gene, herein designated VGAM is inhibition of expression of VGAM332 target genes. It is appreciated that specific functions, and accordingly utilities, of VGAM332 correlate with, and may be deduced from, the identity of the target genes which VGAM332 binds and inhibits, and the function of these target genes, as elaborated hereinbelow.

Desmocollin 3 (DSC3, Accession NM_{—}001941) is a VGAM332 host target gene. DSC3 BINDING SITE1 and DSC3 BINDING SITE2 are HOST TARGET binding sites found in untranslated regions of mRNA encoded by DSC3, corresponding to HOST TARGET binding sites such as BINDING SITE I, BINDING SITE II or BINDING SITE III. Table 2 illustrates the complementarity of the nucleotide sequences of DSC3 BINDING SITE1 and DSC3 BINDING SITE2, designated SEQ ID:871 and SEQ ID:2060 respectively, to the nucleotide sequence of VGAM332 RNA, herein designated VGAM RNA, also designated SEQ ID:667.

A function of VGAM332 is therefore inhibition of Desmocollin 3 (DSC3, Accession NM_{—}001941), a gene which is a component of intercellular desmosome junctions. Accordingly, utilities of VGAM332 include diagnosis, prevention and treatment of diseases and clinical conditions associated with DSC3. The function of DSC3 and its association with various diseases and clinical conditions, has been established by previous studies, as described hereinabove with reference to VGAM306. FIG. 1 further provides a conceptual description of a novel bioinformatically detected viral gene of the present invention, referred to here as Viral Genomic Address Messenger 333 (VGAM333) viral gene, which modulates expression of respective host target genes thereof, the function and utility of which host target genes is known in the art.

VGAM333 is a novel bioinformatically detected regulatory, non protein coding, viral micro RNA (miRNA) gene. The method by which VGAM333 was detected is described hereinabove with reference to FIGS. 1-8.

VGAM333 gene, herein designated VGAM GENE, is a viral gene contained in the genome of Vaccinia Virus. VGAM333 host target gene, herein designated VGAM HOST TARGET GENE, is a human gene contained in the human genome.

VGAM333 gene encodes a VGAM333 precursor RNA, herein designated VGAM PRECURSOR RNA. Similar to other miRNA genes, and unlike most ordinary genes, VGAM333 precursor RNA does not encode a protein. A nucleotide sequence identical or highly similar to the nucleotide sequence of VGAM333 precursor RNA is designated SEQ ID:319, and is provided hereinbelow with reference to the sequence listing part. Nucleotide sequence SEQ ID:319 is located at position 190758 relative to the genome of Vaccinia Virus.

VGAM333 precursor RNA folds onto itself, forming VGAM333 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM333 folded precursor RNA into VGAM333 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM333 RNA is designated SEQ ID:668, and is provided hereinbelow with reference to the sequence listing part.

VGAM333 host target gene, herein designated VGAM HOST TARGET GENE, encodes a corresponding messenger RNA, VGAM333 host target RNA, herein designated VGAM HOST TARGET RNA. VGAM333 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM333 RNA, herein designated VGAM RNA, binds complementarily to one or more host target binding sites located in untranslated regions of VGAM333 host target RNA, herein designated VGAM HOST TARGET RNA. This complementary binding is due to the fact that the nucleotide sequence of VGAM333 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM333 RNA, herein designated VGAM RNA, may have a different number of host target binding sites in untranslated regions of a VGAM333 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM333 RNA, herein designated VGAM RNA, to host target binding sites on VGAM333 host target RNA, herein designated VGAM HOST TARGET RNA, such as BINDING SITE I, BINDING SITE II and BINDING SITE III, inhibits translation of VGAM333 host target RNA into VGAM333 host target protein, herein designated VGAM HOST TARGET PROTEIN. VGAM host target protein is therefore outlined by a broken line.

It is appreciated that VGAM333 host target gene, herein designated VGAM HOST TARGET GENE, in fact represents a plurality of VGAM333 host target genes. The mRNA of each one of this plurality of VGAM333 host target genes comprises one or more host target binding sites, each having a nucleotide sequence which is at least partly complementary to VGAM333 RNA, herein designated VGAM RNA, and which when bound by VGAM333 RNA causes inhibition of translation of respective one or more VGAM333 host target proteins.

It is further appreciated by one skilled in the art that the mode of translational inhibition illustrated by FIG. 1 with specific reference to translational inhibition exerted by VGAM333 gene, herein designated VGAM GENE, on one or more VGAM333 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM333 is inhibition of expression of host target genes, as part of a novel viral mechanism of attacking a host. Accordingly, utilities of VGAM333 include diagnosis, prevention and treatment of viral infection by Vaccinia Virus. Specific functions, and accordingly utilities, of VGAM333 correlate with, and may be deduced from, the identity of the host target genes which VGAM333 binds and inhibits, and the function of these host target genes, as elaborated hereinbelow.

Nucleotide sequences of the VGAM333 precursor RNA, herein designated VGAM PRECURSOR RNA, and of the ‘diced’ VGAM333 RNA, herein designated VGAM RNA, and a schematic representation of the secondary folding of VGAM333 folded precursor RNA, herein designated VGAM FOLDED PRECURSOR RNA, of VGAM333 are further described hereinbelow with reference to Table 1.

Nucleotide sequences of host target binding sites, such as BINDING SITE-I, BINDING SITE-II and BINDING SITE-III of FIG. 1, found on VGAM333 host target RNA, and schematic representation of the complementarity of each of these host target binding sites to VGAM333 RNA, herein designated VGAM RNA, are described hereinbelow with reference to Table 2.

As mentioned hereinabove with reference to FIG. 1, a function of VGAM333 gene, herein designated VGAM is inhibition of expression of VGAM333 target genes. It is appreciated that specific functions, and accordingly utilities, of VGAM333 correlate with, and may be deduced from, the identity of the target genes which VGAM333 binds and inhibits, and the function of these target genes, as elaborated hereinbelow.

Calpain 2, (m/II) Large Subunit (CAPN2, Accession NM_{—}001748) is a VGAM333 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM333 RNA, herein designated VGAM RNA, also designated SEQ ID:668.

A function of VGAM333 is therefore inhibition of Calpain 2, (m/II) Large Subunit (CAPN2, Accession NM_{—}001748). Accordingly, utilities of VGAM333 include diagnosis, prevention and treatment of diseases and clinical conditions associated with CAPN2. Dishevelled Associated Activator of Morphogenesis 2 (DAAM2, Accession XM_{—}166434) is another VGAM333 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM333 RNA, herein designated VGAM RNA, also designated SEQ ID:668.

Another function of VGAM333 is therefore inhibition of Dishevelled Associated Activator of Morphogenesis 2 (DAAM2, Accession XM_{—}166434), a gene which controls cell polarity and movement during development. Accordingly, utilities of VGAM333 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM333 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM333 RNA, herein designated VGAM RNA, also designated SEQ ID:668.

Another function of VGAM333 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM333 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM333 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM333 RNA, herein designated VGAM RNA, also designated SEQ ID:668.

Another function of VGAM333 is therefore inhibition of KIAA0475 (Accession NM_{—}014864). Accordingly, utilities of VGAM333 include diagnosis, prevention and treatment of diseases and clinical conditions associated with KIAA0475. LOC222671 (Accession XM_{—}167094) is another VGAM333 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM333 RNA, herein designated VGAM RNA, also designated SEQ ID:668.

Another function of VGAM333 is therefore inhibition of LOC222671 (Accession XM_{—}167094). Accordingly, utilities of VGAM333 include diagnosis, prevention and treatment of diseases and clinical conditions associated with LOC222671. LOC254901 (Accession XM_{—}171878) is another VGAM333 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM333 RNA, herein designated VGAM RNA, also designated SEQ ID:668.

Another function of VGAM333 is therefore inhibition of LOC254901 (Accession XM_{—}171878). Accordingly, utilities of VGAM333 include diagnosis, prevention and treatment of diseases and clinical conditions associated with LOC254901. LOC257615 (Accession XM_{—}175293) is another VGAM333 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM333 RNA, herein designated VGAM RNA, also designated SEQ ID:668.

Another function of VGAM333 is therefore inhibition of LOC257615 (Accession XM_{—}175293). Accordingly, utilities of VGAM333 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 334 (VGAM334) viral gene, which modulates expression of respective host target genes thereof, the function and utility of which host target genes is known in the art.

VGAM334 is a novel bioinformatically detected regulatory, non protein coding, viral micro RNA (miRNA) gene. The method by which VGAM334 was detected is described hereinabove with reference to FIGS. 1-8.

VGAM334 gene, herein designated VGAM GENE, is a viral gene contained in the genome of Vaccinia Virus. VGAM334 host target gene, herein designated VGAM HOST TARGET GENE, is a human gene contained in the human genome.

VGAM334 gene encodes a VGAM334 precursor RNA, herein designated VGAM PRECURSOR RNA. Similar to other miRNA genes, and unlike most ordinary genes, VGAM334 precursor RNA does not encode a protein. A nucleotide sequence identical or highly similar to the nucleotide sequence of VGAM334 precursor RNA is designated SEQ ID:320, and is provided hereinbelow with reference to the sequence listing part. Nucleotide sequence SEQ ID:320 is located at position 190758 relative to the genome of Vaccinia Virus.

VGAM334 precursor RNA folds onto itself, forming VGAM334 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM334 folded precursor RNA into VGAM334 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM334 RNA is designated SEQ ID:669, and is provided hereinbelow with reference to the sequence listing part.

VGAM334 host target gene, herein designated VGAM HOST TARGET GENE, encodes a corresponding messenger RNA, VGAM334 host target RNA, herein designated VGAM HOST TARGET RNA. VGAM334 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM334 RNA, herein designated VGAM RNA, binds complementarily to one or more host target binding sites located in untranslated regions of VGAM334 host target RNA, herein designated VGAM HOST TARGET RNA. This complementary binding is due to the fact that the nucleotide sequence of VGAM334 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM334 RNA, herein designated VGAM RNA, may have a different number of host target binding sites in untranslated regions of a VGAM334 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM334 RNA, herein designated VGAM RNA, to host target binding sites on VGAM334 host target RNA, herein designated VGAM HOST TARGET RNA, such as BINDING SITE I, BINDING SITE II and BINDING SITE III, inhibits translation of VGAM334 host target RNA into VGAM334 host target protein, herein designated VGAM HOST TARGET PROTEIN. VGAM host target protein is therefore outlined by a broken line.

It is appreciated that VGAM334 host target gene, herein designated VGAM HOST TARGET GENE, in fact represents a plurality of VGAM334 host target genes. The mRNA of each one of this plurality of VGAM334 host target genes comprises one or more host target binding sites, each having a nucleotide sequence which is at least partly complementary to VGAM334 RNA, herein designated VGAM RNA, and which when bound by VGAM334 RNA causes inhibition of translation of respective one or more VGAM334 host target proteins.

It is further appreciated by one skilled in the art that the mode of translational inhibition illustrated by FIG. 1 with specific reference to translational inhibition exerted by VGAM334 gene, herein designated VGAM GENE, on one or more VGAM334 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM334 is inhibition of expression of host target genes, as part of a novel viral mechanism of attacking a host. Accordingly, utilities of VGAM334 include diagnosis, prevention and treatment of viral infection by Vaccinia Virus. Specific functions, and accordingly utilities, of VGAM334 correlate with, and may be deduced from, the identity of the host target genes which VGAM334 binds and inhibits, and the function of these host target genes, as elaborated hereinbelow.

Nucleotide sequences of the VGAM334 precursor RNA, herein designated VGAM PRECURSOR RNA, and of the ‘diced’ VGAM334 RNA, herein designated VGAM RNA, and a schematic representation of the secondary folding of VGAM334 folded precursor RNA, herein designated VGAM FOLDED PRECURSOR RNA, of VGAM334 are further described hereinbelow with reference to Table 1.

Nucleotide sequences of host target binding sites, such as BINDING SITE-I, BINDING SITE-II and BINDING SITE-III of FIG. 1, found on VGAM334 host target RNA, and schematic representation of the complementarity of each of these host target binding sites to VGAM334 RNA, herein designated VGAM RNA, are described hereinbelow with reference to Table 2.

As mentioned hereinabove with reference to FIG. 1, a function of VGAM334 gene, herein designated VGAM is inhibition of expression of VGAM334 target genes. It is appreciated that specific functions, and accordingly utilities, of VGAM334 correlate with, and may be deduced from, the identity of the target genes which VGAM334 binds and inhibits, and the function of these target genes, as elaborated hereinbelow.

Calpain 2, (m/II) Large Subunit (CAPN2, Accession NM_{—}001748) is a VGAM334 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM334 RNA, herein designated VGAM RNA, also designated SEQ ID:669.

A function of VGAM334 is therefore inhibition of Calpain 2, (m/II) Large Subunit (CAPN2, Accession NM_{—}001748). Accordingly, utilities of VGAM334 include diagnosis, prevention and treatment of diseases and clinical conditions associated with CAPN2. Dishevelled Associated Activator of Morphogenesis 2 (DAAM2, Accession XM_{—}166434) is another VGAM334 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM334 RNA, herein designated VGAM RNA, also designated SEQ ID:669.

Another function of VGAM334 is therefore inhibition of Dishevelled Associated Activator of Morphogenesis 2 (DAAM2, Accession XM_{—}166434), a gene which controls cell polarity and movement during development. Accordingly, utilities of VGAM334 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM334 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM334 RNA, herein designated VGAM RNA, also designated SEQ ID:669.

Another function of VGAM334 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM334 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM334 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM334 RNA, herein designated VGAM RNA, also designated SEQ ID:669.

Another function of VGAM334 is therefore inhibition of KIAA0475 (Accession NM_{—}014864). Accordingly, utilities of VGAM334 include diagnosis, prevention and treatment of diseases and clinical conditions associated with KIAA0475. LOC222671 (Accession XM_{—}167094) is another VGAM334 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM334 RNA, herein designated VGAM RNA, also designated SEQ ID:669.

Another function of VGAM334 is therefore inhibition of LOC222671 (Accession XM_{—}167094). Accordingly, utilities of VGAM334 include diagnosis, prevention and treatment of diseases and clinical conditions associated with LOC222671. LOC254901 (Accession XM_{—}171878) is another VGAM334 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM334 RNA, herein designated VGAM RNA, also designated SEQ ID:669.

Another function of VGAM334 is therefore inhibition of LOC254901 (Accession XM_{—}171878). Accordingly, utilities of VGAM334 include diagnosis, prevention and treatment of diseases and clinical conditions associated with LOC254901. LOC257615 (Accession XM_{—}175293) is another VGAM334 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM334 RNA, herein designated VGAM RNA, also designated SEQ ID:669.

Another function of VGAM334 is therefore inhibition of LOC257615 (Accession XM_{—}175293). Accordingly, utilities of VGAM334 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 335 (VGAM335) viral gene, which modulates expression of respective host target genes thereof, the function and utility of which host target genes is known in the art.

VGAM335 is a novel bioinformatically detected regulatory, non protein coding, viral micro RNA (miRNA) gene. The method by which VGAM335 was detected is described hereinabove with reference to FIGS. 1-8.

VGAM335 gene, herein designated VGAM GENE, is a viral gene contained in the genome of Vaccinia Virus. VGAM335 host target gene, herein designated VGAM HOST TARGET GENE, is a human gene contained in the human genome.

VGAM335 gene encodes a VGAM335 precursor RNA, herein designated VGAM PRECURSOR RNA. Similar to other miRNA genes, and unlike most ordinary genes, VGAM335 precursor RNA does not encode a protein. A nucleotide sequence identical or highly similar to the nucleotide sequence of VGAM335 precursor RNA is designated SEQ ID:321, and is provided hereinbelow with reference to the sequence listing part. Nucleotide sequence SEQ ID:321 is located at position 190758 relative to the genome of Vaccinia Virus.

VGAM335 precursor RNA folds onto itself, forming VGAM335 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM335 folded precursor RNA into VGAM335 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 75%) nucleotide sequence of VGAM335 RNA is designated SEQ ID:670, and is provided hereinbelow with reference to the sequence listing part.

VGAM335 host target gene, herein designated VGAM HOST TARGET GENE, encodes a corresponding messenger RNA, VGAM335 host target RNA, herein designated VGAM HOST TARGET RNA. VGAM335 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM335 RNA, herein designated VGAM RNA, binds complementarily to one or more host target binding sites located in untranslated regions of VGAM335 host target RNA, herein designated VGAM HOST TARGET RNA. This complementary binding is due to the fact that the nucleotide sequence of VGAM335 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM335 RNA, herein designated VGAM RNA, may have a different number of host target binding sites in untranslated regions of a VGAM335 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM335 RNA, herein designated VGAM RNA, to host target binding sites on VGAM335 host target RNA, herein designated VGAM HOST TARGET RNA, such as BINDING SITE I, BINDING SITE II and BINDING SITE III, inhibits translation of VGAM335 host target RNA into VGAM335 host target protein, herein designated VGAM HOST TARGET PROTEIN. VGAM host target protein is therefore outlined by a broken line.

It is appreciated that VGAM335 host target gene, herein designated VGAM HOST TARGET GENE, in fact represents a plurality of VGAM335 host target genes. The mRNA of each one of this plurality of VGAM335 host target genes comprises one or more host target binding sites, each having a nucleotide sequence which is at least partly complementary to VGAM335 RNA, herein designated VGAM RNA, and which when bound by VGAM335 RNA causes inhibition of translation of respective one or more VGAM335 host target proteins.

It is further appreciated by one skilled in the art that the mode of translational inhibition illustrated by FIG. 1 with specific reference to translational inhibition exerted by VGAM335 gene, herein designated VGAM GENE, on one or more VGAM335 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM335 is inhibition of expression of host target genes, as part of a novel viral mechanism of attacking a host. Accordingly, utilities of VGAM335 include diagnosis, prevention and treatment of viral infection by Vaccinia Virus. Specific functions, and accordingly utilities, of VGAM335 correlate with, and may be deduced from, the identity of the host target genes which VGAM335 binds and inhibits, and the function of these host target genes, as elaborated hereinbelow.

Nucleotide sequences of the VGAM335 precursor RNA, herein designated VGAM PRECURSOR RNA, and of the ‘diced’ VGAM335 RNA, herein designated VGAM RNA, and a schematic representation of the secondary folding of VGAM335 folded precursor RNA, herein designated VGAM FOLDED PRECURSOR RNA, of VGAM335 are further described hereinbelow with reference to Table 1.

Nucleotide sequences of host target binding sites, such as BINDING SITE-I, BINDING SITE-II and BINDING SITE-III of FIG. 1, found on VGAM335 host target RNA, and schematic representation of the complementarity of each of these host target binding sites to VGAM335 RNA, herein designated VGAM RNA, are described hereinbelow with reference to Table 2.

As mentioned hereinabove with reference to FIG. 1, a function of VGAM335 gene, herein designated VGAM is inhibition of expression of VGAM335 target genes. It is appreciated that specific functions, and accordingly utilities, of VGAM335 correlate with, and may be deduced from, the identity of the target genes which VGAM335 binds and inhibits, and the function of these target genes, as elaborated hereinbelow.

Calpain 2, (m/II) Large Subunit (CAPN2, Accession NM_{—}001748) is a VGAM335 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM335 RNA, herein designated VGAM RNA, also designated SEQ ID:670.

A function of VGAM335 is therefore inhibition of Calpain 2, (m/II) Large Subunit (CAPN2, Accession NM_{—}001748). Accordingly, utilities of VGAM335 include diagnosis, prevention and treatment of diseases and clinical conditions associated with CAPN2. Dishevelled Associated Activator of Morphogenesis 2 (DAAM2, Accession XM_{—}166434) is another VGAM335 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM335 RNA, herein designated VGAM RNA, also designated SEQ ID:670.

Another function of VGAM335 is therefore inhibition of Dishevelled Associated Activator of Morphogenesis 2 (DAAM2, Accession XM_{—}166434), a gene which controls cell polarity and movement during development. Accordingly, utilities of VGAM335 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM335 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM335 RNA, herein designated VGAM RNA, also designated SEQ ID:670.

Another function of VGAM335 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM335 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM335 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM335 RNA, herein designated VGAM RNA, also designated SEQ ID:670.

Another function of VGAM335 is therefore inhibition of KIAA0475 (Accession NM_{—}014864). Accordingly, utilities of VGAM335 include diagnosis, prevention and treatment of diseases and clinical conditions associated with KIAA0475. LOC222671 (Accession XM_{—}167094) is another VGAM335 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM335 RNA, herein designated VGAM RNA, also designated SEQ ID:670.

Another function of VGAM335 is therefore inhibition of LOC222671 (Accession XM_{—}167094). Accordingly, utilities of VGAM335 include diagnosis, prevention and treatment of diseases and clinical conditions associated with LOC222671. LOC254901 (Accession XM_{—}171878) is another VGAM335 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM335 RNA, herein designated VGAM RNA, also designated SEQ ID:670.

Another function of VGAM335 is therefore inhibition of LOC254901 (Accession XM_{—}171878). Accordingly, utilities of VGAM335 include diagnosis, prevention and treatment of diseases and clinical conditions associated with LOC254901. LOC257615 (Accession XM_{—}175293) is another VGAM335 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM335 RNA, herein designated VGAM RNA, also designated SEQ ID:670.

Another function of VGAM335 is therefore inhibition of LOC257615 (Accession XM_{—}175293). Accordingly, utilities of VGAM335 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 336 (VGAM336) viral gene, which modulates expression of respective host target genes thereof, the function and utility of which host target genes is known in the art.

VGAM336 is a novel bioinformatically detected regulatory, non protein coding, viral micro RNA (miRNA) gene. The method by which VGAM336 was detected is described hereinabove with reference to FIGS. 1-8.

VGAM336 gene, herein designated VGAM GENE, is a viral gene contained in the genome of Vaccinia Virus. VGAM336 host target gene, herein designated VGAM HOST TARGET GENE, is a human gene contained in the human genome.

VGAM336 gene encodes a VGAM336 precursor RNA, herein designated VGAM PRECURSOR RNA. Similar to other miRNA genes, and unlike most ordinary genes, VGAM336 precursor RNA does not encode a protein. A nucleotide sequence identical or highly similar to the nucleotide sequence of VGAM336 precursor RNA is designated SEQ ID:322, and is provided hereinbelow with reference to the sequence listing part. Nucleotide sequence SEQ ID:322 is located at position 188937 relative to the genome of Vaccinia Virus.

VGAM336 precursor RNA folds onto itself, forming VGAM336 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM336 folded precursor RNA into VGAM336 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 77%) nucleotide sequence of VGAM336 RNA is designated SEQ ID:671, and is provided hereinbelow with reference to the sequence listing part.

VGAM336 host target gene, herein designated VGAM HOST TARGET GENE, encodes a corresponding messenger RNA, VGAM336 host target RNA, herein designated VGAM HOST TARGET RNA. VGAM336 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM336 RNA, herein designated VGAM RNA, binds complementarily to one or more host target binding sites located in untranslated regions of VGAM336 host target RNA, herein designated VGAM HOST TARGET RNA. This complementary binding is due to the fact that the nucleotide sequence of VGAM336 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM336 RNA, herein designated VGAM RNA, may have a different number of host target binding sites in untranslated regions of a VGAM336 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM336 RNA, herein designated VGAM RNA, to host target binding sites on VGAM336 host target RNA, herein designated VGAM HOST TARGET RNA, such as BINDING SITE I, BINDING SITE II and BINDING SITE III, inhibits translation of VGAM336 host target RNA into VGAM336 host target protein, herein designated VGAM HOST TARGET PROTEIN. VGAM host target protein is therefore outlined by a broken line.

It is appreciated that VGAM336 host target gene, herein designated VGAM HOST TARGET GENE, in fact represents a plurality of VGAM336 host target genes. The mRNA of each one of this plurality of VGAM336 host target genes comprises one or more host target binding sites, each having a nucleotide sequence which is at least partly complementary to VGAM336 RNA, herein designated VGAM RNA, and which when bound by VGAM336 RNA causes inhibition of translation of respective one or more VGAM336 host target proteins.

It is further appreciated by one skilled in the art that the mode of translational inhibition illustrated by FIG. 1 with specific reference to translational inhibition exerted by VGAM336 gene, herein designated VGAM GENE, on one or more VGAM336 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM336 is inhibition of expression of host target genes, as part of a novel viral mechanism of attacking a host. Accordingly, utilities of VGAM336 include diagnosis, prevention and treatment of viral infection by Vaccinia Virus. Specific functions, and accordingly utilities, of VGAM336 correlate with, and may be deduced from, the identity of the host target genes which VGAM336 binds and inhibits, and the function of these host target genes, as elaborated hereinbelow.

Nucleotide sequences of the VGAM336 precursor RNA, herein designated VGAM PRECURSOR RNA, and of the ‘diced’ VGAM336 RNA, herein designated VGAM RNA, and a schematic representation of the secondary folding of VGAM336 folded precursor RNA, herein designated VGAM FOLDED PRECURSOR RNA, of VGAM336 are further described hereinbelow with reference to Table 1.

Nucleotide sequences of host target binding sites, such as BINDING SITE-I, BINDING SITE-II and BINDING SITE-III of FIG. 1, found on VGAM336 host target RNA, and schematic representation of the complementarity of each of these host target binding sites to VGAM336 RNA, herein designated VGAM RNA, are described hereinbelow with reference to Table 2.

As mentioned hereinabove with reference to FIG. 1, a function of VGAM336 gene, herein designated VGAM is inhibition of expression of VGAM336 target genes. It is appreciated that specific functions, and accordingly utilities, of VGAM336 correlate with, and may be deduced from, the identity of the target genes which VGAM336 binds and inhibits, and the function of these target genes, as elaborated hereinbelow.

Calpain 2, (m/II) Large Subunit (CAPN2, Accession NM_{—}001748) is a VGAM336 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM336 RNA, herein designated VGAM RNA, also designated SEQ ID:671.

A function of VGAM336 is therefore inhibition of Calpain 2, (m/II) Large Subunit (CAPN2, Accession NM_{—}001748). Accordingly, utilities of VGAM336 include diagnosis, prevention and treatment of diseases and clinical conditions associated with CAPN2. Dishevelled Associated Activator of Morphogenesis 2 (DAAM2, Accession XM_{—}166434) is another VGAM336 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM336 RNA, herein designated VGAM RNA, also designated SEQ ID:671.

Another function of VGAM336 is therefore inhibition of Dishevelled Associated Activator of Morphogenesis 2 (DAAM2, Accession XM_{—}166434), a gene which controls cell polarity and movement during development. Accordingly, utilities of VGAM336 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM336 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM336 RNA, herein designated VGAM RNA, also designated SEQ ID:671.

Another function of VGAM336 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM336 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM336 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM336 RNA, herein designated VGAM RNA, also designated SEQ ID:671.

Another function of VGAM336 is therefore inhibition of KIAA0475 (Accession NM_{—}014864). Accordingly, utilities of VGAM336 include diagnosis, prevention and treatment of diseases and clinical conditions associated with KIAA0475. LOC222671 (Accession XM_{—}167094) is another VGAM336 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM336 RNA, herein designated VGAM RNA, also designated SEQ ID:671.

Another function of VGAM336 is therefore inhibition of LOC222671 (Accession XM_{—}167094). Accordingly, utilities of VGAM336 include diagnosis, prevention and treatment of diseases and clinical conditions associated with LOC222671. LOC254901 (Accession XM_{—}171878) is another VGAM336 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM336 RNA, herein designated VGAM RNA, also designated SEQ ID:671.

Another function of VGAM336 is therefore inhibition of LOC254901 (Accession XM_{—}171878). Accordingly, utilities of VGAM336 include diagnosis, prevention and treatment of diseases and clinical conditions associated with LOC254901. LOC257615 (Accession XM_{—}175293) is another VGAM336 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM336 RNA, herein designated VGAM RNA, also designated SEQ ID:671.

Another function of VGAM336 is therefore inhibition of LOC257615 (Accession XM_{—}175293). Accordingly, utilities of VGAM336 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 337 (VGAM337) viral gene, which modulates expression of respective host target genes thereof, the function and utility of which host target genes is known in the art.

VGAM337 is a novel bioinformatically detected regulatory, non protein coding, viral micro RNA (miRNA) gene. The method by which VGAM337 was detected is described hereinabove with reference to FIGS. 1-8.

VGAM337 gene, herein designated VGAM GENE, is a viral gene contained in the genome of Vaccinia Virus. VGAM337 host target gene, herein designated VGAM HOST TARGET GENE, is a human gene contained in the human genome.

VGAM337 gene encodes a VGAM337 precursor RNA, herein designated VGAM PRECURSOR RNA. Similar to other miRNA genes, and unlike most ordinary genes, VGAM337 precursor RNA does not encode a protein. A nucleotide sequence identical or highly similar to the nucleotide sequence of VGAM337 precursor RNA is designated SEQ ID:323, and is provided hereinbelow with reference to the sequence listing part. Nucleotide sequence SEQ ID:323 is located at position 187719 relative to the genome of Vaccinia Virus.

VGAM337 precursor RNA folds onto itself, forming VGAM337 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM337 folded precursor RNA into VGAM337 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 88%) nucleotide sequence of VGAM337 RNA is designated SEQ ID:672, and is provided hereinbelow with reference to the sequence listing part.

VGAM337 host target gene, herein designated VGAM HOST TARGET GENE, encodes a corresponding messenger RNA, VGAM337 host target RNA, herein designated VGAM HOST TARGET RNA. VGAM337 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM337 RNA, herein designated VGAM RNA, binds complementarily to one or more host target binding sites located in untranslated regions of VGAM337 host target RNA, herein designated VGAM HOST TARGET RNA. This complementary binding is due to the fact that the nucleotide sequence of VGAM337 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM337 RNA, herein designated VGAM RNA, may have a different number of host target binding sites in untranslated regions of a VGAM337 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM337 RNA, herein designated VGAM RNA, to host target binding sites on VGAM337 host target RNA, herein designated VGAM HOST TARGET RNA, such as BINDING SITE I, BINDING SITE II and BINDING SITE III, inhibits translation of VGAM337 host target RNA into VGAM337 host target protein, herein designated VGAM HOST TARGET PROTEIN. VGAM host target protein is therefore outlined by a broken line.

It is appreciated that VGAM337 host target gene, herein designated VGAM HOST TARGET GENE, in fact represents a plurality of VGAM337 host target genes. The mRNA of each one of this plurality of VGAM337 host target genes comprises one or more host target binding sites, each having a nucleotide sequence which is at least partly complementary to VGAM337 RNA, herein designated VGAM RNA, and which when bound by VGAM337 RNA causes inhibition of translation of respective one or more VGAM337 host target proteins.

It is further appreciated by one skilled in the art that the mode of translational inhibition illustrated by FIG. 1 with specific reference to translational inhibition exerted by VGAM337 gene, herein designated VGAM GENE, on one or more VGAM337 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM337 is inhibition of expression of host target genes, as part of a novel viral mechanism of attacking a host. Accordingly, utilities of VGAM337 include diagnosis, prevention and treatment of viral infection by Vaccinia Virus. Specific functions, and accordingly utilities, of VGAM337 correlate with, and may be deduced from, the identity of the host target genes which VGAM337 binds and inhibits, and the function of these host target genes, as elaborated hereinbelow.

Nucleotide sequences of the VGAM337 precursor RNA, herein designated VGAM PRECURSOR RNA, and of the ‘diced’ VGAM337 RNA, herein designated VGAM RNA, and a schematic representation of the secondary folding of VGAM337 folded precursor RNA, herein designated VGAM FOLDED PRECURSOR RNA, of VGAM337 are further described hereinbelow with reference to Table 1.

Nucleotide sequences of host target binding sites, such as BINDING SITE-I, BINDING SITE-II and BINDING SITE-III of FIG. 1, found on VGAM337 host target RNA, and schematic representation of the complementarity of each of these host target binding sites to VGAM337 RNA, herein designated VGAM RNA, are described hereinbelow with reference to Table 2.

As mentioned hereinabove with reference to FIG. 1, a function of VGAM337 gene, herein designated VGAM is inhibition of expression of VGAM337 target genes. It is appreciated that specific functions, and accordingly utilities, of VGAM337 correlate with, and may be deduced from, the identity of the target genes which VGAM337 binds and inhibits, and the function of these target genes, as elaborated hereinbelow.

Fibroblast Growth Factor 2 (basic) (FGF2, Accession NM_{—}002006) is a VGAM337 host target gene. FGF2 BINDING SITE is HOST TARGET binding site found in the 3′ untranslated region of mRNA encoded by FGF2, corresponding to a HOST TARGET binding site such as BINDING SITE I, BINDING SITE II or BINDING SITE III. Table 2 illustrates the complementarity of the nucleotide sequences of FGF2 BINDING SITE, designated SEQ ID:884, to the nucleotide sequence of VGAM337 RNA, herein designated VGAM RNA, also designated SEQ ID:672.

A function of VGAM337 is therefore inhibition of Fibroblast Growth Factor 2 (basic) (FGF2, Accession NM_{—}002006), a gene which Basic fibroblast growth factor 2. Accordingly, utilities of VGAM337 include diagnosis, prevention and treatment of diseases and clinical conditions associated with FGF2. The function of FGF2 and its association with various diseases and clinical conditions, has been established by previous studies, as described hereinabove with reference to VGAM30. M-phase Phosphoprotein 1 (MPHOSPH1, Accession NM_{—}016195) is another VGAM337 host target gene. MPHOSPH1 BINDING SITE is HOST TARGET binding site found in the 3′ untranslated region of mRNA encoded by MPHOSPH1, corresponding to a HOST TARGET binding site such as BINDING SITE I, BINDING SITE II or BINDING SITE III. Table 2 illustrates the complementarity of the nucleotide sequences of MPHOSPH1 BINDING SITE, designated SEQ ID:1670, to the nucleotide sequence of VGAM337 RNA, herein designated VGAM RNA, also designated SEQ ID:672.

Another function of VGAM337 is therefore inhibition of M-phase Phosphoprotein 1 (MPHOSPH1, Accession NM_{—}016195), a gene which is Phosphorylated during M-phase and interacts with guanosine triphosphate. Accordingly, utilities of VGAM337 include diagnosis, prevention and treatment of diseases and clinical conditions associated with MPHOSPH1. The function of MPHOSPH1 and its association with various diseases and clinical conditions, has been established by previous studies, as described hereinabove with reference to VGAM30. FLJ11274 (Accession NM_{—}018375) is another VGAM337 host target gene. FLJ11274 BINDING SITE is HOST TARGET binding site found in the 3′ untranslated region of mRNA encoded by FLJ11274, corresponding to a HOST TARGET binding site such as BINDING SITE I, BINDING SITE II or BINDING SITE III. Table 2 illustrates the complementarity of the nucleotide sequences of FLJ11274 BINDING SITE, designated SEQ ID: 1820, to the nucleotide sequence of VGAM337 RNA, herein designated VGAM RNA, also designated SEQ ID:672.

Another function of VGAM337 is therefore inhibition of FLJ11274 (Accession NM_{—}018375). Accordingly, utilities of VGAM337 include diagnosis, prevention and treatment of diseases and clinical conditions associated with FLJ11274. FLJ31101 (Accession NM_{—}017964) is another VGAM337 host target gene. FLJ31101 BINDING SITE is HOST TARGET binding site found in the 3′ untranslated region of mRNA encoded by FLJ31101, corresponding to a HOST TARGET binding site such as BINDING SITE I, BINDING SITE II or BINDING SITE III. Table 2 illustrates the complementarity of the nucleotide sequences of FLJ31101 BINDING SITE, designated SEQ ID:1771, to the nucleotide sequence of VGAM337 RNA, herein designated VGAM RNA, also designated SEQ ID:672.

Another function of VGAM337 is therefore inhibition of FLJ31101 (Accession NM_{—}017964). Accordingly, utilities of VGAM337 include diagnosis, prevention and treatment of diseases and clinical conditions associated with FLJ31101. Glutamic Pyruvate Transaminase (alanine aminotransferase) 2 (GPT2, Accession NM_{—}133443) is another VGAM337 host target gene. GPT2 BINDING SITE is HOST TARGET binding site found in the 3′ untranslated region of mRNA encoded by GPT2, corresponding to a HOST TARGET binding site such as BINDING SITE I, BINDING SITE II or BINDING SITE III. Table 2 illustrates the complementarity of the nucleotide sequences of GPT2 BINDING SITE, designated SEQ ID:2417, to the nucleotide sequence of VGAM337 RNA, herein designated VGAM RNA, also designated SEQ ID:672.

Another function of VGAM337 is therefore inhibition of Glutamic Pyruvate Transaminase (alanine aminotransferase) 2 (GPT2, Accession NM_{—}133443). Accordingly, utilities of VGAM337 include diagnosis, prevention and treatment of diseases and clinical conditions associated with GPT2. RMP (Accession NM_{—}134447) is another VGAM337 host target gene. RMP BINDING SITE1 and RMP BINDING SITE2 are HOST TARGET binding sites found in untranslated regions of mRNA encoded by RMP, corresponding to HOST TARGET binding sites such as BINDING SITE I, BINDING SITE II or BINDING SITE III. Table 2 illustrates the complementarity of the nucleotide sequences of RMP BINDING SITE1 and RMP BINDING SITE2, designated SEQ ID:2430 and SEQ ID: 1059 respectively, to the nucleotide sequence of VGAM337 RNA, herein designated VGAM RNA, also designated SEQ ID:672.

Another function of VGAM337 is therefore inhibition of RMP (Accession NM_{—}134447). Accordingly, utilities of VGAM337 include diagnosis, prevention and treatment of diseases and clinical conditions associated with RMP. LOC136288 (Accession XM_{—}059832) is another VGAM337 host target gene. LOC136288 BINDING SITE is HOST TARGET binding site found in the 3′ untranslated region of mRNA encoded by LOC136288, corresponding to a HOST TARGET binding site such as BINDING SITE I, BINDING SITE II or BINDING SITE III. Table 2 illustrates the complementarity of the nucleotide sequences of LOC136288 BINDING SITE, designated SEQ ID:3018, to the nucleotide sequence of VGAM337 RNA, herein designated VGAM RNA, also designated SEQ ID:672.

Another function of VGAM337 is therefore inhibition of LOC136288 (Accession XM_{—}059832). Accordingly, utilities of VGAM337 include diagnosis, prevention and treatment of diseases and clinical conditions associated with LOC136288. FIG. 1 further provides a conceptual description of a novel bioinformatically detected viral gene of the present invention, referred to here as Viral Genomic Address Messenger 338 (VGAM338) viral gene, which modulates expression of respective host target genes thereof, the function and utility of which host target genes is known in the art.

VGAM338 is a novel bioinformatically detected regulatory, non protein coding, viral micro RNA (miRNA) gene. The method by which VGAM338 was detected is described hereinabove with reference to FIGS. 1-8.

VGAM338 gene, herein designated VGAM GENE, is a viral gene contained in the genome of Vaccinia Virus. VGAM338 host target gene, herein designated VGAM HOST TARGET GENE, is a human gene contained in the human genome.

VGAM338 gene encodes a VGAM338 precursor RNA, herein designated VGAM PRECURSOR RNA. Similar to other miRNA genes, and unlike most ordinary genes, VGAM338 precursor RNA does not encode a protein. A nucleotide sequence identical or highly similar to the nucleotide sequence of VGAM338 precursor RNA is designated SEQ ID:324, and is provided hereinbelow with reference to the sequence listing part. Nucleotide sequence SEQ ID:324 is located at position 188420 relative to the genome of Vaccinia Virus.

VGAM338 precursor RNA folds onto itself, forming VGAM338 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM338 folded precursor RNA into VGAM338 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM338 RNA is designated SEQ ID:673, and is provided hereinbelow with reference to the sequence listing part.

VGAM338 host target gene, herein designated VGAM HOST TARGET GENE, encodes a corresponding messenger RNA, VGAM338 host target RNA, herein designated VGAM HOST TARGET RNA. VGAM338 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM338 RNA, herein designated VGAM RNA, binds complementarily to one or more host target binding sites located in untranslated regions of VGAM338 host target RNA, herein designated VGAM HOST TARGET RNA. This complementary binding is due to the fact that the nucleotide sequence of VGAM338 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM338 RNA, herein designated VGAM RNA, may have a different number of host target binding sites in untranslated regions of a VGAM338 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM338 RNA, herein designated VGAM RNA, to host target binding sites on VGAM338 host target RNA, herein designated VGAM HOST TARGET RNA, such as BINDING SITE I, BINDING SITE II and BINDING SITE III, inhibits translation of VGAM338 host target RNA into VGAM338 host target protein, herein designated VGAM HOST TARGET PROTEIN. VGAM host target protein is therefore outlined by a broken line.

It is appreciated that VGAM338 host target gene, herein designated VGAM HOST TARGET GENE, in fact represents a plurality of VGAM338 host target genes. The mRNA of each one of this plurality of VGAM338 host target genes comprises one or more host target binding sites, each having a nucleotide sequence which is at least partly complementary to VGAM338 RNA, herein designated VGAM RNA, and which when bound by VGAM338 RNA causes inhibition of translation of respective one or more VGAM338 host target proteins.

It is further appreciated by one skilled in the art that the mode of translational inhibition illustrated by FIG. 1 with specific reference to translational inhibition exerted by VGAM338 gene, herein designated VGAM GENE, on one or more VGAM338 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM338 is inhibition of expression of host target genes, as part of a novel viral mechanism of attacking a host. Accordingly, utilities of VGAM338 include diagnosis, prevention and treatment of viral infection by Vaccinia Virus. Specific functions, and accordingly utilities, of VGAM338 correlate with, and may be deduced from, the identity of the host target genes which VGAM338 binds and inhibits, and the function of these host target genes, as elaborated hereinbelow.

Nucleotide sequences of the VGAM338 precursor RNA, herein designated VGAM PRECURSOR RNA, and of the ‘diced’ VGAM338 RNA, herein designated VGAM RNA, and a schematic representation of the secondary folding of VGAM338 folded precursor RNA, herein designated VGAM FOLDED PRECURSOR RNA, of VGAM338 are further described hereinbelow with reference to Table 1.

Nucleotide sequences of host target binding sites, such as BINDING SITE-I, BINDING SITE-II and BINDING SITE-III of FIG. 1, found on VGAM338 host target RNA, and schematic representation of the complementarity of each of these host target binding sites to VGAM338 RNA, herein designated VGAM RNA, are described hereinbelow with reference to Table 2.

As mentioned hereinabove with reference to FIG. 1, a function of VGAM338 gene, herein designated VGAM is inhibition of expression of VGAM338 target genes. It is appreciated that specific functions, and accordingly utilities, of VGAM338 correlate with, and may be deduced from, the identity of the target genes which VGAM338 binds and inhibits, and the function of these target genes, as elaborated hereinbelow.

Activation-induced Cytidine Deaminase (AICDA, Accession NM_{—}020661) is a VGAM338 host target gene. AICDA BINDING SITE is HOST TARGET binding site found in the 3′ untranslated region of mRNA encoded by AICDA, corresponding to a HOST TARGET binding site such as BINDING SITE I, BINDING SITE II or BINDING SITE III. Table 2 illustrates the complementarity of the nucleotide sequences of AICDA BINDING SITE, designated SEQ ID:1922, to the nucleotide sequence of VGAM338 RNA, herein designated VGAM RNA, also designated SEQ ID:673.

A function of VGAM338 is therefore inhibition of Activation-induced Cytidine Deaminase (AICDA, Accession NM_{—}020661), a gene which a member of the cytidine deaminase family. Accordingly, utilities of VGAM338 include diagnosis, prevention and treatment of diseases and clinical conditions associated with AICDA. The function of AICDA and its association with various diseases and clinical conditions, has been established by previous studies, as described hereinabove with reference to VGAM31. AS3 (Accession NM_{—}015928) is another VGAM338 host target gene. AS3 BINDING SITE is HOST TARGET binding site found in the 3′ untranslated region of mRNA encoded by AS3, corresponding to a HOST TARGET binding site such as BINDING SITE I, BINDING SITE II or BINDING SITE III. Table 2 illustrates the complementarity of the nucleotide sequences of AS3 BINDING SITE, designated SEQ ID: 1652, to the nucleotide sequence of VGAM338 RNA, herein designated VGAM RNA, also designated SEQ ID:673.

Another function of VGAM338 is therefore inhibition of AS3 (Accession NM_{—}015928), a gene which inhibits cell proloferation. Accordingly, utilities of VGAM338 include diagnosis, prevention and treatment of diseases and clinical conditions associated with AS3. The function of AS3 and its association with various diseases and clinical conditions, has been established by previous studies, as described hereinabove with reference to VGAM31. Epilepsy, Progressive Myoclonus Type 2, Lafora Disease (laforin) (EPM2A, Accession NM_{—}005670) is another VGAM338 host target gene. EPM2A BINDING SITE is HOST TARGET binding site found in the 3′ untranslated region of mRNA encoded by EPM2A, corresponding to a HOST TARGET binding site such as BINDING SITE I, BINDING SITE II or BINDING SITE III. Table 2 illustrates the complementarity of the nucleotide sequences of EPM2A BINDING SITE, designated SEQ ID:1234, to the nucleotide sequence of VGAM338 RNA, herein designated VGAM RNA, also designated SEQ ID:673.

Another function of VGAM338 is therefore inhibition of Epilepsy, Progressive Myoclonus Type 2, Lafora Disease (laforin) (EPM2A, Accession NM_{—}005670), a gene which Laforin; protein tyrosine phosphatase that may have role in glycogen metabolism. Accordingly, utilities of VGAM338 include diagnosis, prevention and treatment of diseases and clinical conditions associated with EPM2A. The function of EPM2A and its association with various diseases and clinical conditions, has been established by previous studies, as described hereinabove with reference to VGAM31. Histamine Receptor H1 (HRH1, Accession NM_{—}000861) is another VGAM338 host target gene. HRH1 BINDING SITE is HOST TARGET binding site found in the 3′ untranslated region of mRNA encoded by HRH1, corresponding to a HOST TARGET binding site such as BINDING SITE I, BINDING SITE II or BINDING SITE III. Table 2 illustrates the complementarity of the nucleotide sequences of HRH1 BINDING SITE, designated SEQ ID:780, to the nucleotide sequence of VGAM338 RNA, herein designated VGAM RNA, also designated SEQ ID:673.

Another function of VGAM338 is therefore inhibition of Histamine Receptor H1 (HRH1, Accession NM_{—}000861), a gene which stimulates the synthesis of inositol phosphate. Accordingly, utilities of VGAM338 include diagnosis, prevention and treatment of diseases and clinical conditions associated with HRH1. The function of HRH1 and its association with various diseases and clinical conditions, has been established by previous studies, as described hereinabove with reference to VGAM31. Mitogen-activated Protein Kinase 14 (MAPK14, Accession NM_{—}001315) is another VGAM338 host target gene. MAPK14 BINDING SITE1 through MAPK14 BINDING SITE3 are HOST TARGET binding sites found in untranslated regions of mRNA encoded by MAPK14, corresponding to HOST TARGET binding sites such as BINDING SITE I, BINDING SITE II or BINDING SITE III. Table 2 illustrates the complementarity of the nucleotide sequences of MAPK14 BINDING SITE1 through MAPK14 BINDING SITE3, designated SEQ ID:818, SEQ ID:2465 and SEQ ID:2466 respectively, to the nucleotide sequence of VGAM338 RNA, herein designated VGAM RNA, also designated SEQ ID:673.

Another function of VGAM338 is therefore inhibition of Mitogen-activated Protein Kinase 14 (MAPK14, Accession NM_{—}001315), a gene which is important for cytokine production; responds to changes in extracellular osmolarity. Accordingly, utilities of VGAM338 include diagnosis, prevention and treatment of diseases and clinical conditions associated with MAPK14. The function of MAPK14 and its association with various diseases and clinical conditions, has been established by previous studies, as described hereinabove with reference to VGAM31. Single-minded Homolog 1 (Drosophila) (SIM1, Accession NM_{—}005068) is another VGAM338 host target gene. SIM1 BINDING SITE is HOST TARGET binding site found in the 3′ untranslated region of mRNA encoded by SIM1, corresponding to a HOST TARGET binding site such as BINDING SITE I, BINDING SITE II or BINDING SITE III. Table 2 illustrates the complementarity of the nucleotide sequences of SIM1 BINDING SITE, designated SEQ ID:1181, to the nucleotide sequence of VGAM338 RNA, herein designated VGAM RNA, also designated SEQ ID:673.

Another function of VGAM338 is therefore inhibition of Single-minded Homolog 1 (Drosophila) (SIM1, Accession NM_{—}005068), a gene which may have pleiotropic effects during embryogenesis and in the adult. Accordingly, utilities of VGAM338 include diagnosis, prevention and treatment of diseases and clinical conditions associated with SIM1. The function of SIM1 and its association with various diseases and clinical conditions, has been established by previous studies, as described hereinabove with reference to VGAM31. Chromosome 11 Open Reading Frame 25 (C11orf25, Accession NM_{—}031418) is another VGAM338 host target gene. C11orf25 BINDING SITE is HOST TARGET binding site found in the 3′ untranslated region of mRNA encoded by C11orf25, corresponding to a HOST TARGET binding site such as BINDING SITE I, BINDING SITE II or BINDING SITE III. Table 2 illustrates the complementarity of the nucleotide sequences of C11orf25 BINDING SITE, designated SEQ ID:2193, to the nucleotide sequence of VGAM338 RNA, herein designated VGAM RNA, also designated SEQ ID:673.

Another function of VGAM338 is therefore inhibition of Chromosome 11 Open Reading Frame 25 (C11orf25, Accession NM_{—}031418). Accordingly, utilities of VGAM338 include diagnosis, prevention and treatment of diseases and clinical conditions associated with C11orf25. DKFZP564D116 (Accession XM_{—}051050) is another VGAM338 host target gene. DKFZP564D116 BINDING SITE is HOST TARGET binding site found in the 3′ untranslated region of mRNA encoded by DKFZP564D116, corresponding to a HOST TARGET binding site such as BINDING SITE I, BINDING SITE II or BINDING SITE III. Table 2 illustrates the complementarity of the nucleotide sequences of DKFZP564D116 BINDING SITE, designated SEQ ID:2938, to the nucleotide sequence of VGAM338 RNA, herein designated VGAM RNA, also designated SEQ ID:673.

Another function of VGAM338 is therefore inhibition of DKFZP564D116 (Accession XM_{—}051050). Accordingly, utilities of VGAM338 include diagnosis, prevention and treatment of diseases and clinical conditions associated with DKFZP564D116. Down Syndrome Critical Region Gene 6 (DSCR6, Accession NM_{—}018962) is another VGAM338 host target gene. DSCR6 BINDING SITE is HOST TARGET binding site found in the 3′ untranslated region of mRNA encoded by DSCR6, corresponding to a HOST TARGET binding site such as BINDING SITE I, BINDING SITE II or BINDING SITE III. Table 2 illustrates the complementarity of the nucleotide sequences of DSCR6 BINDING SITE, designated SEQ ID:1866, to the nucleotide sequence of VGAM338 RNA, herein designated VGAM RNA, also designated SEQ ID:673.

Another function of VGAM338 is therefore inhibition of Down Syndrome Critical Region Gene 6 (DSCR6, Accession NM_{—}018962). Accordingly, utilities of VGAM338 include diagnosis, prevention and treatment of diseases and clinical conditions associated with DSCR6. FLJ10006 (Accession XM_{—}087073) is another VGAM338 host target gene. FLJ10006 BINDING SITE is HOST TARGET binding site found in the 3′ untranslated region of mRNA encoded by FLJ10006, corresponding to a HOST TARGET binding site such as BINDING SITE I, BINDING SITE II or BINDING SITE III. Table 2 illustrates the complementarity of the nucleotide sequences of FLJ10006 BINDING SITE, designated SEQ ID:3152, to the nucleotide sequence of VGAM338 RNA, herein designated VGAM RNA, also designated SEQ ID:673.

Another function of VGAM338 is therefore inhibition of FLJ10006 (Accession XM_{—}087073). Accordingly, utilities of VGAM338 include diagnosis, prevention and treatment of diseases and clinical conditions associated with FLJ10006. HTPAP (Accession NM_{—}032483) is another VGAM338 host target gene. HTPAP BINDING SITE is HOST TARGET binding site found in the 3′ untranslated region of mRNA encoded by HTPAP, corresponding to a HOST TARGET binding site such as BINDING SITE I, BINDING SITE II or BINDING SITE III. Table 2 illustrates the complementarity of the nucleotide sequences of HTPAP BINDING SITE, designated SEQ ID:2255, to the nucleotide sequence of VGAM338 RNA, herein designated VGAM RNA, also designated SEQ ID:673.

Another function of VGAM338 is therefore inhibition of HTPAP (Accession NM_{—}032483). Accordingly, utilities of VGAM338 include diagnosis, prevention and treatment of diseases and clinical conditions associated with HTPAP. KIAA0979 (Accession NM_{—}015032) is another VGAM338 host target gene. KIAA0979 BINDING SITE is HOST TARGET binding site found in the 3′ untranslated region of mRNA encoded by KIAA0979, corresponding to a HOST TARGET binding site such as BINDING SITE I, BINDING SITE II or BINDING SITE III. Table 2 illustrates the complementarity of the nucleotide sequences of KIAA0979 BINDING SITE, designated SEQ ID: 1601, to the nucleotide sequence of VGAM338 RNA, herein designated VGAM RNA, also designated SEQ ID:673.

Another function of VGAM338 is therefore inhibition of KIAA0979 (Accession NM_{—}015032). Accordingly, utilities of VGAM338 include diagnosis, prevention and treatment of diseases and clinical conditions associated with KIAA0979. KIAA1634 (Accession XM_{—}032749) is another VGAM338 host target gene. KIAA1634 BINDING SITE is HOST TARGET binding site found in the 3′ untranslated region of mRNA encoded by KIAA1634, corresponding to a HOST TARGET binding site such as BINDING SITE I, BINDING SITE II or BINDING SITE III. Table 2 illustrates the complementarity of the nucleotide sequences of KIAA1634 BINDING SITE, designated SEQ ID:2657, to the nucleotide sequence of VGAM338 RNA, herein designated VGAM RNA, also designated SEQ ID:673.

Another function of VGAM338 is therefore inhibition of KIAA1634 (Accession XM_{—}032749). Accordingly, utilities of VGAM338 include diagnosis, prevention and treatment of diseases and clinical conditions associated with KIAA1634. PTD012 (Accession NM_{—}014039) is another VGAM338 host target gene. PTD012 BINDING SITE is HOST TARGET binding site found in the 3′ untranslated region of mRNA encoded by PTD012, corresponding to a HOST TARGET binding site such as BINDING SITE I, BINDING SITE II or BINDING SITE III. Table 2 illustrates the complementarity of the nucleotide sequences of PTD012 BINDING SITE, designated SEQ ID: 1465, to the nucleotide sequence of VGAM338 RNA, herein designated VGAM RNA, also designated SEQ ID:673.

Another function of VGAM338 is therefore inhibition of PTD012 (Accession NM_{—}014039). Accordingly, utilities of VGAM338 include diagnosis, prevention and treatment of diseases and clinical conditions associated with PTD012. SAM Domain and HD Domain 1 (SAMHD1, Accession XM_{—}028704) is another VGAM338 host target gene. SAMHD1 BINDING SITE is HOST TARGET binding site found in the 3′ untranslated region of mRNA encoded by SAMHD1, corresponding to a HOST TARGET binding site such as BINDING SITE I, BINDING SITE II or BINDING SITE III. Table 2 illustrates the complementarity of the nucleotide sequences of SAMHD1 BINDING SITE, designated SEQ ID:2593, to the nucleotide sequence of VGAM338 RNA, herein designated VGAM RNA, also designated SEQ ID:673.

Another function of VGAM338 is therefore inhibition of SAM Domain and HD Domain 1 (SAMHD1, Accession XM_{—}028704). Accordingly, utilities of VGAM338 include diagnosis, prevention and treatment of diseases and clinical conditions associated with SAMHD1. LOC196758 (Accession XM_{—}113600) is another VGAM338 host target gene. LOC196758 BINDING SITE is HOST TARGET binding site found in the 3′ untranslated region of mRNA encoded by LOC196758, corresponding to a HOST TARGET binding site such as BINDING SITE I, BINDING SITE II or BINDING SITE III. Table 2 illustrates the complementarity of the nucleotide sequences of LOC196758 BINDING SITE, designated SEQ ID:3410, to the nucleotide sequence of VGAM338 RNA, herein designated VGAM RNA, also designated SEQ ID:673.

Another function of VGAM338 is therefore inhibition of LOC196758 (Accession XM_{—}113600). Accordingly, utilities of VGAM338 include diagnosis, prevention and treatment of diseases and clinical conditions associated with LOC196758. LOC219790 (Accession XM_{—}166124) is another VGAM338 host target gene. LOC219790 BINDING SITE is HOST TARGET binding site found in the 3′ untranslated region of mRNA encoded by LOC219790, corresponding to a HOST TARGET binding site such as BINDING SITE I, BINDING SITE II or BINDING SITE III. Table 2 illustrates the complementarity of the nucleotide sequences of LOC219790 BINDING SITE, designated SEQ ID:3520, to the nucleotide sequence of VGAM338 RNA, herein designated VGAM RNA, also designated SEQ ID:673.

Another function of VGAM338 is therefore inhibition of LOC219790 (Accession XM_{—}166124). Accordingly, utilities of VGAM338 include diagnosis, prevention and treatment of diseases and clinical conditions associated with LOC219790. FIG. 1 further provides a conceptual description of a novel bioinformatically detected viral gene of the present invention, referred to here as Viral Genomic Address Messenger 339 (VGAM339) viral gene, which modulates expression of respective host target genes thereof, the function and utility of which host target genes is known in the art.

VGAM339 is a novel bioinformatically detected regulatory, non protein coding, viral micro RNA (miRNA) gene. The method by which VGAM339 was detected is described hereinabove with reference to FIGS. 1-8.

VGAM339 gene, herein designated VGAM GENE, is a viral gene contained in the genome of Vaccinia Virus. VGAM339 host target gene, herein designated VGAM HOST TARGET GENE, is a human gene contained in the human genome.

VGAM339 gene encodes a VGAM339 precursor RNA, herein designated VGAM PRECURSOR RNA. Similar to other miRNA genes, and unlike most ordinary genes, VGAM339 precursor RNA does not encode a protein. A nucleotide sequence identical or highly similar to the nucleotide sequence of VGAM339 precursor RNA is designated SEQ ID:325, and is provided hereinbelow with reference to the sequence listing part. Nucleotide sequence SEQ ID:325 is located at position 190813 relative to the genome of Vaccinia Virus.

VGAM339 precursor RNA folds onto itself, forming VGAM339 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM339 folded precursor RNA into VGAM339 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 82%) nucleotide sequence of VGAM339 RNA is designated SEQ ID:674, and is provided hereinbelow with reference to the sequence listing part.

VGAM339 host target gene, herein designated VGAM HOST TARGET GENE, encodes a corresponding messenger RNA, VGAM339 host target RNA, herein designated VGAM HOST TARGET RNA. VGAM339 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM339 RNA, herein designated VGAM RNA, binds complementarily to one or more host target binding sites located in untranslated regions of VGAM339 host target RNA, herein designated VGAM HOST TARGET RNA. This complementary binding is due to the fact that the nucleotide sequence of VGAM339 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM339 RNA, herein designated VGAM RNA, may have a different number of host target binding sites in untranslated regions of a VGAM339 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM339 RNA, herein designated VGAM RNA, to host target binding sites on VGAM339 host target RNA, herein designated VGAM HOST TARGET RNA, such as BINDING SITE I, BINDING SITE II and BINDING SITE III, inhibits translation of VGAM339 host target RNA into VGAM339 host target protein, herein designated VGAM HOST TARGET PROTEIN. VGAM host target protein is therefore outlined by a broken line.

It is appreciated that VGAM339 host target gene, herein designated VGAM HOST TARGET GENE, in fact represents a plurality of VGAM339 host target genes. The mRNA of each one of this plurality of VGAM339 host target genes comprises one or more host target binding sites, each having a nucleotide sequence which is at least partly complementary to VGAM339 RNA, herein designated VGAM RNA, and which when bound by VGAM339 RNA causes inhibition of translation of respective one or more VGAM339 host target proteins.

It is further appreciated by one skilled in the art that the mode of translational inhibition illustrated by FIG. 1 with specific reference to translational inhibition exerted by VGAM339 gene, herein designated VGAM GENE, on one or more VGAM339 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM339 is inhibition of expression of host target genes, as part of a novel viral mechanism of attacking a host. Accordingly, utilities of VGAM339 include diagnosis, prevention and treatment of viral infection by Vaccinia Virus. Specific functions, and accordingly utilities, of VGAM339 correlate with, and may be deduced from, the identity of the host target genes which VGAM339 binds and inhibits, and the function of these host target genes, as elaborated hereinbelow.

Nucleotide sequences of the VGAM339 precursor RNA, herein designated VGAM PRECURSOR RNA, and of the ‘diced’ VGAM339 RNA, herein designated VGAM RNA, and a schematic representation of the secondary folding of VGAM339 folded precursor RNA, herein designated VGAM FOLDED PRECURSOR RNA, of VGAM339 are further described hereinbelow with reference to Table 1.

Nucleotide sequences of host target binding sites, such as BINDING SITE-I, BINDING SITE-II and BINDING SITE-III of FIG. 1, found on VGAM339 host target RNA, and schematic representation of the complementarity of each of these host target binding sites to VGAM339 RNA, herein designated VGAM RNA, are described hereinbelow with reference to Table 2.

As mentioned hereinabove with reference to FIG. 1, a function of VGAM339 gene, herein designated VGAM is inhibition of expression of VGAM339 target genes. It is appreciated that specific functions, and accordingly utilities, of VGAM339 correlate with, and may be deduced from, the identity of the target genes which VGAM339 binds and inhibits, and the function of these target genes, as elaborated hereinbelow.

Calpain 2, (m/II) Large Subunit (CAPN2, Accession NM_{—}001748) is a VGAM339 host target gene. CAPN2 BINDING SITE is HOST TARGET binding site found in the 3′ untranslated region of mRNA encoded by CAPN2, corresponding to a HOST TARGET binding site such as BINDING SITE I, BINDING SITE II or BINDING SITE III. Table 2 illustrates the complementarity of the nucleotide sequences of CAPN2 BINDING SITE, designated SEQ ID:855, to the nucleotide sequence of VGAM339 RNA, herein designated VGAM RNA, also designated SEQ ID:674.

A function of VGAM339 is therefore inhibition of Calpain 2, (m/II) Large Subunit (CAPN2, Accession NM_{—}001748). Accordingly, utilities of VGAM339 include diagnosis, prevention and treatment of diseases and clinical conditions associated with CAPN2. Dishevelled Associated Activator of Morphogenesis 2 (DAAM2, Accession XM_{—}166434) is another VGAM339 host target gene. DAAM2 BINDING SITE is HOST TARGET binding site found in the 3′ untranslated region of mRNA encoded by DAAM2, corresponding to a HOST TARGET binding site such as BINDING SITE I, BINDING SITE II or BINDING SITE III. Table 2 illustrates the complementarity of the nucleotide sequences of DAAM2 BINDING SITE, designated SEQ ID:3559, to the nucleotide sequence of VGAM339 RNA, herein designated VGAM RNA, also designated SEQ ID:674.

Another function of VGAM339 is therefore inhibition of Dishevelled Associated Activator of Morphogenesis 2 (DAAM2, Accession XM_{—}166434), a gene which controls cell polarity and movement during development. Accordingly, utilities of VGAM339 include diagnosis, prevention and treatment of diseases and clinical conditions associated with DAAM2. The function of DAAM2 and its association with various diseases and clinical conditions, has been established by previous studies, as described hereinabove with reference to VGAM15. Selectin E (endothelial adhesion molecule 1) (SELE, Accession NM_{—}000450) is another VGAM339 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:747, to the nucleotide sequence of VGAM339 RNA, herein designated VGAM RNA, also designated SEQ ID:674.

Another function of VGAM339 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM339 include diagnosis, prevention and treatment of diseases and clinical conditions associated with SELE. The function of SELE and its association with various diseases and clinical conditions, has been established by previous studies, as described hereinabove with reference to VGAM15. KIAA0475 (Accession NM_{—}014864) is another VGAM339 host target gene. KIAA0475 BINDING SITE is HOST TARGET binding site found in the 3′ untranslated region of mRNA encoded by KIAA0475, corresponding to a HOST TARGET binding site such as BINDING SITE I, BINDING SITE II or BINDING SITE III. Table 2 illustrates the complementarity of the nucleotide sequences of KIAA0475 BINDING SITE, designated SEQ ID: 1570, to the nucleotide sequence of VGAM339 RNA, herein designated VGAM RNA, also designated SEQ ID:674.

Another function of VGAM339 is therefore inhibition of KIAA0475 (Accession NM_{—}014864). Accordingly, utilities of VGAM339 include diagnosis, prevention and treatment of diseases and clinical conditions associated with KIAA0475. LOC222671 (Accession XM_{—}167094) is another VGAM339 host target gene. LOC222671 BINDING SITE is HOST TARGET binding site found in the 5′ untranslated region of mRNA encoded by LOC222671, corresponding to a HOST TARGET binding site such as BINDING SITE I, BINDING SITE II or BINDING SITE III. Table 2 illustrates the complementarity of the nucleotide sequences of LOC222671 BINDING SITE, designated SEQ ID:3582, to the nucleotide sequence of VGAM339 RNA, herein designated VGAM RNA, also designated SEQ ID:674.

Another function of VGAM339 is therefore inhibition of LOC222671 (Accession XM_{—}167094). Accordingly, utilities of VGAM339 include diagnosis, prevention and treatment of diseases and clinical conditions associated with LOC222671. LOC254901 (Accession XM_{—}171878) is another VGAM339 host target gene. LOC254901 BINDING SITE is HOST TARGET binding site found in the 5′ untranslated region of mRNA encoded by LOC254901, corresponding to a HOST TARGET binding site such as BINDING SITE I, BINDING SITE II or BINDING SITE III. Table 2 illustrates the complementarity of the nucleotide sequences of LOC254901 BINDING SITE, designated SEQ ID:3699, to the nucleotide sequence of VGAM339 RNA, herein designated VGAM RNA, also designated SEQ ID:674.

Another function of VGAM339 is therefore inhibition of LOC254901 (Accession XM_{—}171878). Accordingly, utilities of VGAM339 include diagnosis, prevention and treatment of diseases and clinical conditions associated with LOC254901. LOC257615 (Accession XM_{—}175293) is another VGAM339 host target gene. LOC257615 BINDING SITE is HOST TARGET binding site found in the 5′ untranslated region of mRNA encoded by LOC257615, corresponding to a HOST TARGET binding site such as BINDING SITE I, BINDING SITE II or BINDING SITE III. Table 2 illustrates the complementarity of the nucleotide sequences of LOC257615 BINDING SITE, designated SEQ ID:3749, to the nucleotide sequence of VGAM339 RNA, herein designated VGAM RNA, also designated SEQ ID:674.

Another function of VGAM339 is therefore inhibition of LOC257615 (Accession XM_{—}175293). Accordingly, utilities of VGAM339 include diagnosis, prevention and treatment of diseases and clinical conditions associated with LOC257615. FIG. 1 further provides a conceptual description of a novel bioinformatically detected viral gene of the present invention, referred to here as Viral Genomic Address Messenger 340 (VGAM340) viral gene, which modulates expression of respective host target genes thereof, the function and utility of which host target genes is known in the art.

VGAM340 is a novel bioinformatically detected regulatory, non protein coding, viral micro RNA (miRNA) gene. The method by which VGAM340 was detected is described hereinabove with reference to FIGS. 1-8.

VGAM340 gene, herein designated VGAM GENE, is a viral gene contained in the genome of Vaccinia Virus. VGAM340 host target gene, herein designated VGAM HOST TARGET GENE, is a human gene contained in the human genome.

VGAM340 gene encodes a VGAM340 precursor RNA, herein designated VGAM PRECURSOR RNA. Similar to other miRNA genes, and unlike most ordinary genes, VGAM340 precursor RNA does not encode a protein. A nucleotide sequence identical or highly similar to the nucleotide sequence of VGAM340 precursor RNA is designated SEQ ID:326, and is provided hereinbelow with reference to the sequence listing part. Nucleotide sequence SEQ ID:326 is located at position 188778 relative to the genome of Vaccinia Virus.

VGAM340 precursor RNA folds onto itself, forming VGAM340 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM340 folded precursor RNA into VGAM340 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 90%) nucleotide sequence of VGAM340 RNA is designated SEQ ID:675, and is provided hereinbelow with reference to the sequence listing part.

VGAM340 host target gene, herein designated VGAM HOST TARGET GENE, encodes a corresponding messenger RNA, VGAM340 host target RNA, herein designated VGAM HOST TARGET RNA. VGAM340 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM340 RNA, herein designated VGAM RNA, binds complementarily to one or more host target binding sites located in untranslated regions of VGAM340 host target RNA, herein designated VGAM HOST TARGET RNA. This complementary binding is due to the fact that the nucleotide sequence of VGAM340 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM340 RNA, herein designated VGAM RNA, may have a different number of host target binding sites in untranslated regions of a VGAM340 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM340 RNA, herein designated VGAM RNA, to host target binding sites on VGAM340 host target RNA, herein designated VGAM HOST TARGET RNA, such as BINDING SITE I, BINDING SITE II and BINDING SITE III, inhibits translation of VGAM340 host target RNA into VGAM340 host target protein, herein designated VGAM HOST TARGET PROTEIN. VGAM host target protein is therefore outlined by a broken line.

It is appreciated that VGAM340 host target gene, herein designated VGAM HOST TARGET GENE, in fact represents a plurality of VGAM340 host target genes. The mRNA of each one of this plurality of VGAM340 host target genes comprises one or more host target binding sites, each having a nucleotide sequence which is at least partly complementary to VGAM340 RNA, herein designated VGAM RNA, and which when bound by VGAM340 RNA causes inhibition of translation of respective one or more VGAM340 host target proteins.

It is further appreciated by one skilled in the art that the mode of translational inhibition illustrated by FIG. 1 with specific reference to translational inhibition exerted by VGAM340 gene, herein designated VGAM GENE, on one or more VGAM340 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM340 is inhibition of expression of host target genes, as part of a novel viral mechanism of attacking a host. Accordingly, utilities of VGAM340 include diagnosis, prevention and treatment of viral infection by Vaccinia Virus. Specific functions, and accordingly utilities, of VGAM340 correlate with, and may be deduced from, the identity of the host target genes which VGAM340 binds and inhibits, and the function of these host target genes, as elaborated hereinbelow.

Nucleotide sequences of the VGAM340 precursor RNA, herein designated VGAM PRECURSOR RNA, and of the ‘diced’ VGAM340 RNA, herein designated VGAM RNA, and a schematic representation of the secondary folding of VGAM340 folded precursor RNA, herein designated VGAM FOLDED PRECURSOR RNA, of VGAM340 are further described hereinbelow with reference to Table 1.

Nucleotide sequences of host target binding sites, such as BINDING SITE-I, BINDING SITE-II and BINDING SITE-III of FIG. 1, found on VGAM340 host target RNA, and schematic representation of the complementarity of each of these host target binding sites to VGAM340 RNA, herein designated VGAM RNA, are described hereinbelow with reference to Table 2.

As mentioned hereinabove with reference to FIG. 1, a function of VGAM340 gene, herein designated VGAM is inhibition of expression of VGAM340 target genes. It is appreciated that specific functions, and accordingly utilities, of VGAM340 correlate with, and may be deduced from, the identity of the target genes which VGAM340 binds and inhibits, and the function of these target genes, as elaborated hereinbelow.

Adenylate Cyclase 6 (ADCY6, Accession NM_{—}015270) is a VGAM340 host target gene. ADCY6 BINDING SITE1 and ADCY6 BINDING SITE2 are HOST TARGET binding sites found in untranslated regions of mRNA encoded by ADCY6, corresponding to HOST TARGET binding sites such as BINDING SITE I, BINDING SITE II or BINDING SITE III. Table 2 illustrates the complementarity of the nucleotide sequences of ADCY6 BINDING SITE1 and ADCY6 BINDING SITE2, designated SEQ ID: 1617 and SEQ ID: 1933 respectively, to the nucleotide sequence of VGAM340 RNA, herein designated VGAM RNA, also designated SEQ ID:675.

A function of VGAM340 is therefore inhibition of Adenylate Cyclase 6 (ADCY6, Accession NM_{—}015270), a gene which this a membrane-bound, ca(2+)-inhibitable adenylyl cyclase (by similarity). Accordingly, utilities of VGAM340 include diagnosis, prevention and treatment of diseases and clinical conditions associated with ADCY6. The function of ADCY6 and its association with various diseases and clinical conditions, has been established by previous studies, as described hereinabove with reference to VGAM34. ATPase, Class I, Type 8B, Member 2 (ATP8B2, Accession XM_{—}036933) is another VGAM340 host target gene. ATP8B2 BINDING SITE is HOST TARGET binding site found in the 3′ untranslated region of mRNA encoded by ATP8B2, corresponding to a HOST TARGET binding site such as BINDING SITE I, BINDING SITE II or BINDING SITE III. Table 2 illustrates the complementarity of the nucleotide sequences of ATP8B2 BINDING SITE, designated SEQ ID:2716, to the nucleotide sequence of VGAM340 RNA, herein designated VGAM RNA, also designated SEQ ID:675.

Another function of VGAM340 is therefore inhibition of ATPase, Class I, Type 8B, Member 2 (ATP8B2, Accession XM_{—}036933). Accordingly, utilities of VGAM340 include diagnosis, prevention and treatment of diseases and clinical conditions associated with ATP8B2. Carcinoembryonic Antigen-related Cell Adhesion Molecule 1 (biliary glycoprotein) (CEACAM1, Accession NM_{—}001712) is another VGAM340 host target gene. CEACAM1 BINDING SITE is HOST TARGET binding site found in the 3′ untranslated region of mRNA encoded by CEACAM1, corresponding to a HOST TARGET binding site such as BINDING SITE I, BINDING SITE II or BINDING SITE III. Table 2 illustrates the complementarity of the nucleotide sequences of CEACAM1 BINDING SITE, designated SEQ ID:848, to the nucleotide sequence of VGAM340 RNA, herein designated VGAM RNA, also designated SEQ ID:675.

Another function of VGAM340 is therefore inhibition of Carcinoembryonic Antigen-related Cell Adhesion Molecule 1 (biliary glycoprotein) (CEACAM1, Accession NM_{—}001712), a gene which is a major effector of VEGF and may be a target for the inhibition of tumor angiogenesis. Accordingly, utilities of VGAM340 include diagnosis, prevention and treatment of diseases and clinical conditions associated with CEACAM1. The function of CEACAM1 and its association with various diseases and clinical conditions, has been established by previous studies, as described hereinabove with reference to VGAM34. Carbohydrate (keratan sulfate Gal-6) Sulfotransferase 1 (CHST1, Accession NM_{—}003654) is another VGAM340 host target gene. CHST1 BINDING SITE is HOST TARGET binding site found in the 3′ untranslated region of mRNA encoded by CHST1, corresponding to a HOST TARGET binding site such as BINDING SITE I, BINDING SITE II or BINDING SITE III. Table 2 illustrates the complementarity of the nucleotide sequences of CHST1 BINDING SITE, designated SEQ ID: 1046, to the nucleotide sequence of VGAM340 RNA, herein designated VGAM RNA, also designated SEQ ID:675.

Another function of VGAM340 is therefore inhibition of Carbohydrate (keratan sulfate Gal-6) Sulfotransferase 1 (CHST1, Accession NM_{—}003654), a gene which may play a role in keratan sulfate biosynthesis in brain and cornea. Accordingly, utilities of VGAM340 include diagnosis, prevention and treatment of diseases and clinical conditions associated with CHST1. The function of CHST1 and its association with various diseases and clinical conditions, has been established by previous studies, as described hereinabove with reference to VGAM34. F-box and Leucine-rich Repeat Protein 7 (FBXL7, Accession NM_{—}012304) is another VGAM340 host target gene. FBXL7 BINDING SITE is HOST TARGET binding site found in the 3′ untranslated region of mRNA encoded by FBXL7, corresponding to a HOST TARGET binding site such as BINDING SITE I, BINDING SITE II or BINDING SITE III. Table 2 illustrates the complementarity of the nucleotide sequences of FBXL7 BINDING SITE, designated SEQ ID:1425, to the nucleotide sequence of VGAM340 RNA, herein designated VGAM RNA, also designated SEQ ID:675.

Another function of VGAM340 is therefore inhibition of F-box and Leucine-rich Repeat Protein 7 (FBXL7, Accession NM_{—}012304), a gene which may be involved in phosphorylation-dependent ubiquitination. Accordingly, utilities of VGAM340 include diagnosis, prevention and treatment of diseases and clinical conditions associated with FBXL7. The function of FBXL7 and its association with various diseases and clinical conditions, has been established by previous studies, as described hereinabove with reference to VGAM34. Homeo Box C9 (HOXC9, Accession XM_{—}028620) is another VGAM340 host target gene. HOXC9 BINDING SITE is HOST TARGET binding site found in the 3′ untranslated region of mRNA encoded by HOXC9, corresponding to a HOST TARGET binding site such as BINDING SITE I, BINDING SITE II or BINDING SITE III. Table 2 illustrates the complementarity of the nucleotide sequences of HOXC9 BINDING SITE, designated SEQ ID:2592, to the nucleotide sequence of VGAM340 RNA, herein designated VGAM RNA, also designated SEQ ID:675.

Another function of VGAM340 is therefore inhibition of Homeo Box C9 (HOXC9, Accession XM_{—}028620). Accordingly, utilities of VGAM340 include diagnosis, prevention and treatment of diseases and clinical conditions associated with HOXC9. Insulin-like Growth Factor 1 (somatomedin C) (IGF1, Accession NM_{—}000618) is another VGAM340 host target gene. IGF1 BINDING SITE is HOST TARGET binding site found in the 3′ untranslated region of mRNA encoded by IGF1, corresponding to a HOST TARGET binding site such as BINDING SITE I, BINDING SITE II or BINDING SITE III. Table 2 illustrates the complementarity of the nucleotide sequences of IGF1 BINDING SITE, designated SEQ ID:763, to the nucleotide sequence of VGAM340 RNA, herein designated VGAM RNA, also designated SEQ ID:675.

Another function of VGAM340 is therefore inhibition of Insulin-like Growth Factor 1 (somatomedin C) (IGF1, Accession NM_{—}000618), a gene which are structurally and functionally related to insulin but have a much higher growth-promoting activity. Accordingly, utilities of VGAM340 include diagnosis, prevention and treatment of diseases and clinical conditions associated with IGF1. The function of IGF1 and its association with various diseases and clinical conditions, has been established by previous studies, as described hereinabove with reference to VGAM34. Integrin, Alpha L (antigen CD11A (p180), Lymphocyte Function-associated Antigen 1; Alpha Polypeptide) (ITGAL, Accession NM_{—}002209) is another VGAM340 host target gene. ITGAL BINDING SITE is HOST TARGET binding site found in the 3′ untranslated region of mRNA encoded by ITGAL, corresponding to a HOST TARGET binding site such as BINDING SITE I, BINDING SITE II or BINDING SITE III. Table 2 illustrates the complementarity of the nucleotide sequences of ITGAL BINDING SITE, designated SEQ ID:907, to the nucleotide sequence of VGAM340 RNA, herein designated VGAM RNA, also designated SEQ ID:675.

Another function of VGAM340 is therefore inhibition of Integrin, Alpha L (antigen CD11A (p180), Lymphocyte Function-associated Antigen 1; Alpha Polypeptide) (ITGAL, Accession NM_{—}002209), a gene which s a receptor for icam1, icam2, icam3 and icam4. it is involved in a variety of immune phenomena including leukocyte-endothelial cell interaction, cytotoxic t-cell mediated killing, and antibody dependent killing by granulocytes and monocytes. Accordingly, utilities of VGAM340 include diagnosis, prevention and treatment of diseases and clinical conditions associated with ITGAL. The function of ITGAL and its association with various diseases and clinical conditions, has been established by previous studies, as described hereinabove with reference to VGAM34. Lamin B Receptor (LBR, Accession XM_{—}001795) is another VGAM340 host target gene. LBR BINDING SITE is HOST TARGET binding site found in the 3′ untranslated region of mRNA encoded by LBR, corresponding to a HOST TARGET binding site such as BINDING SITE I, BINDING SITE II or BINDING SITE III. Table 2 illustrates the complementarity of the nucleotide sequences of LBR BINDING SITE, designated SEQ ID:2522, to the nucleotide sequence of VGAM340 RNA, herein designated VGAM RNA, also designated SEQ ID:675.

Another function of VGAM340 is therefore inhibition of Lamin B Receptor (LBR, Accession XM_{—}001795). Accordingly, utilities of VGAM340 include diagnosis, prevention and treatment of diseases and clinical conditions associated with LBR. Major Intrinsic Protein of Lens Fiber (MIP, Accession NM_{—}012064) is another VGAM340 host target gene. MIP BINDING SITE is HOST TARGET binding site found in the 3′ untranslated region of mRNA encoded by MIP, corresponding to a HOST TARGET binding site such as BINDING SITE I, BINDING SITE II or BINDING SITE III. Table 2 illustrates the complementarity of the nucleotide sequences of MIP BINDING SITE, designated SEQ ID:1399, to the nucleotide sequence of VGAM340 RNA, herein designated VGAM RNA, also designated SEQ ID:675.

Another function of VGAM340 is therefore inhibition of Major Intrinsic Protein of Lens Fiber (MIP, Accession NM_{—}012064). Accordingly, utilities of VGAM340 include diagnosis, prevention and treatment of diseases and clinical conditions associated with MIP. Relaxin 2 (H2) (RLN2, Accession NM_{—}005059) is another VGAM340 host target gene. RLN2 BINDING SITE is HOST TARGET binding site found in the 3′ untranslated region of mRNA encoded by RLN2, corresponding to a HOST TARGET binding site such as BINDING SITE I, BINDING SITE II or BINDING SITE III. Table 2 illustrates the complementarity of the nucleotide sequences of RLN2 BINDING SITE, designated SEQ ID:1175, to the nucleotide sequence of VGAM340 RNA, herein designated VGAM RNA, also designated SEQ ID:675.

Another function of VGAM340 is therefore inhibition of Relaxin 2 (H2) (RLN2, Accession NM_{—}005059). Accordingly, utilities of VGAM340 include diagnosis, prevention and treatment of diseases and clinical conditions associated with RLN2. SART2 (Accession NM_{—}013352) is another VGAM340 host target gene. SART2 BINDING SITE is HOST TARGET binding site found in the 3′ untranslated region of mRNA encoded by SART2, corresponding to a HOST TARGET binding site such as BINDING SITE I, BINDING SITE II or BINDING SITE III. Table 2 illustrates the complementarity of the nucleotide sequences of SART2 BINDING SITE, designated SEQ ID: 1444, to the nucleotide sequence of VGAM340 RNA, herein designated VGAM RNA, also designated SEQ ID:675.

Another function of VGAM340 is therefore inhibition of SART2 (Accession NM_{—}013352). Accordingly, utilities of VGAM340 include diagnosis, prevention and treatment of diseases and clinical conditions associated with SART2. Thiamin Pyrophosphokinase 1 (TPK1, Accession NM_{—}022445) is another VGAM340 host target gene. TPK1 BINDING SITE is HOST TARGET binding site found in the 3′ untranslated region of mRNA encoded by TPK1, corresponding to a HOST TARGET binding site such as BINDING SITE I, BINDING SITE II or BINDING SITE III. Table 2 illustrates the complementarity of the nucleotide sequences of TPK1 BINDING SITE, designated SEQ ID:1987, to the nucleotide sequence of VGAM340 RNA, herein designated VGAM RNA, also designated SEQ ID:675.

Another function of VGAM340 is therefore inhibition of Thiamin Pyrophosphokinase 1 (TPK1, Accession NM_{—}022445), a gene which catalyzes the conversion of thiamine, a form of vitamin B1, to thiamine pyrophosphate. Accordingly, utilities of VGAM340 include diagnosis, prevention and treatment of diseases and clinical conditions associated with TPK1. The function of TPK1 and its association with various diseases and clinical conditions, has been established by previous studies, as described hereinabove with reference to VGAM34. Thyroid Hormone Receptor Interactor 12 (TRIP12, Accession NM_{—}004238) is another VGAM340 host target gene. TRIP12 BINDING SITE is HOST TARGET binding site found in the 3′ untranslated region of mRNA encoded by TRIP12, corresponding to a HOST TARGET binding site such as BINDING SITE I, BINDING SITE II or BINDING SITE III. Table 2 illustrates the complementarity of the nucleotide sequences of TRIP12 BINDING SITE, designated SEQ ID: 1094, to the nucleotide sequence of VGAM340 RNA, herein designated VGAM RNA, also designated SEQ ID:675.

Another function of VGAM340 is therefore inhibition of Thyroid Hormone Receptor Interactor 12 (TRIP12, Accession NM_{—}004238), a gene which interacts with the ligand binding domain of the thyroid hormone receptor (in a thyroid hormone t3-independent manner) and with retinoid×receptor (r×r). Accordingly, utilities of VGAM340 include diagnosis, prevention and treatment of diseases and clinical conditions associated with TRIP12. The function of TRIP12 and its association with various diseases and clinical conditions, has been established by previous studies, as described hereinabove with reference to VGAM34. Bromodomain Containing 3 (BRD3, Accession NM_{—}007371) is another VGAM340 host target gene. BRD3 BINDING SITE is HOST TARGET binding site found in the 3′ untranslated region of mRNA encoded by BRD3, corresponding to a HOST TARGET binding site such as BINDING SITE I, BINDING SITE II or BINDING SITE III. Table 2 illustrates the complementarity of the nucleotide sequences of BRD3 BINDING SITE, designated SEQ ID:1398, to the nucleotide sequence of VGAM340 RNA, herein designated VGAM RNA, also designated SEQ ID:675.

Another function of VGAM340 is therefore inhibition of Bromodomain Containing 3 (BRD3, Accession NM_{—}007371). Accordingly, utilities of VGAM340 include diagnosis, prevention and treatment of diseases and clinical conditions associated with BRD3. FLJ13397 (Accession NM_{—}024948) is another VGAM340 host target gene. FLJ13397 BINDING SITE is HOST TARGET binding site found in the 3′ untranslated region of mRNA encoded by FLJ13397, corresponding to a HOST TARGET binding site such as BINDING SITE I, BINDING SITE II or BINDING SITE III. Table 2 illustrates the complementarity of the nucleotide sequences of FLJ13397 BINDING SITE, designated SEQ ID:2116, to the nucleotide sequence of VGAM340 RNA, herein designated VGAM RNA, also designated SEQ ID:675.

Another function of VGAM340 is therefore inhibition of FLJ13397 (Accession NM_{—}024948). Accordingly, utilities of VGAM340 include diagnosis, prevention and treatment of diseases and clinical conditions associated with FLJ13397. FLJ22202 (Accession NM_{—}024883) is another VGAM340 host target gene. FLJ22202 BINDING SITE is HOST TARGET binding site found in the 3′ untranslated region of mRNA encoded by FLJ22202, corresponding to a HOST TARGET binding site such as BINDING SITE I, BINDING SITE II or BINDING SITE III. Table 2 illustrates the complementarity of the nucleotide sequences of FLJ22202 BINDING SITE, designated SEQ ID:2103, to the nucleotide sequence of VGAM340 RNA, herein designated VGAM RNA, also designated SEQ ID:675.

Another function of VGAM340 is therefore inhibition of FLJ22202 (Accession NM_{—}024883). Accordingly, utilities of VGAM340 include diagnosis, prevention and treatment of diseases and clinical conditions associated with FLJ22202. GROS1 (Accession NM_{—}022356) is another VGAM340 host target gene. GROS1 BINDING SITE is HOST TARGET binding site found in the 3′ untranslated region of mRNA encoded by GROS1, corresponding to a HOST TARGET binding site such as BINDING SITE I, BINDING SITE II or BINDING SITE III. Table 2 illustrates the complementarity of the nucleotide sequences of GROS1 BINDING SITE, designated SEQ ID:1986, to the nucleotide sequence of VGAM340 RNA, herein designated VGAM RNA, also designated SEQ ID:675.

Another function of VGAM340 is therefore inhibition of GROS1 (Accession NM_{—}022356). Accordingly, utilities of VGAM340 include diagnosis, prevention and treatment of diseases and clinical conditions associated with GROS1. KIAA0981 (Accession XM_{—}028867) is another VGAM340 host target gene. KIAA0981 BINDING SITE is HOST TARGET binding site found in the 3′ untranslated region of mRNA encoded by KIAA0981, corresponding to a HOST TARGET binding site such as BINDING SITE I, BINDING SITE II or BINDING SITE III. Table 2 illustrates the complementarity of the nucleotide sequences of KIAA0981 BINDING SITE, designated SEQ ID:2598, to the nucleotide sequence of VGAM340 RNA, herein designated VGAM RNA, also designated SEQ ID:675.

Another function of VGAM340 is therefore inhibition of KIAA0981 (Accession XM_{—}028867). Accordingly, utilities of VGAM340 include diagnosis, prevention and treatment of diseases and clinical conditions associated with KIAA0981. KIAA1243 (Accession XM_{—}057057) is another VGAM340 host target gene. KIAA1243 BINDING SITE is HOST TARGET binding site found in the 3′ untranslated region of mRNA encoded by KIAA1243, corresponding to a HOST TARGET binding site such as BINDING SITE I, BINDING SITE II or BINDING SITE III. Table 2 illustrates the complementarity of the nucleotide sequences of KIAA1243 BINDING SITE, designated SEQ ID:2978, to the nucleotide sequence of VGAM340 RNA, herein designated VGAM RNA, also designated SEQ ID:675.

Another function of VGAM340 is therefore inhibition of KIAA1243 (Accession XM_{—}057057). Accordingly, utilities of VGAM340 include diagnosis, prevention and treatment of diseases and clinical conditions associated with KIAA1243. KIAA1462 (Accession XM_{—}166132) is another VGAM340 host target gene. KIAA1462 BINDING SITE is HOST TARGET binding site found in the 3′ untranslated region of mRNA encoded by KIAA1462, corresponding to a HOST TARGET binding site such as BINDING SITE I, BINDING SITE II or BINDING SITE III. Table 2 illustrates the complementarity of the nucleotide sequences of KIAA1462 BINDING SITE, designated SEQ ID:3524, to the nucleotide sequence of VGAM340 RNA, herein designated VGAM RNA, also designated SEQ ID:675.

Another function of VGAM340 is therefore inhibition of KIAA1462 (Accession XM_{—}166132). Accordingly, utilities of VGAM340 include diagnosis, prevention and treatment of diseases and clinical conditions associated with KIAA1462. MGC1136 (Accession NM_{—}024025) is another VGAM340 host target gene. MGC1136 BINDING SITE is HOST TARGET binding site found in the 3′ untranslated region of mRNA encoded by MGC1136, corresponding to a HOST TARGET binding site such as BINDING SITE I, BINDING SITE II or BINDING SITE III. Table 2 illustrates the complementarity of the nucleotide sequences of MGC1136 BINDING SITE, designated SEQ ID:2041, to the nucleotide sequence of VGAM340 RNA, herein designated VGAM RNA, also designated SEQ ID:675.

Another function of VGAM340 is therefore inhibition of MGC1136 (Accession NM_{—}024025). Accordingly, utilities of VGAM340 include diagnosis, prevention and treatment of diseases and clinical conditions associated with MGC1136. MSTP043 (Accession NM_{—}031953) is another VGAM340 host target gene. MSTP043 BINDING SITE is HOST TARGET binding site found in the 3′ untranslated region of mRNA encoded by MSTP043, corresponding to a HOST TARGET binding site such as BINDING SITE I, BINDING SITE II or BINDING SITE III. Table 2 illustrates the complementarity of the nucleotide sequences of MSTP043 BINDING SITE, designated SEQ ID:2223, to the nucleotide sequence of VGAM340 RNA, herein designated VGAM RNA, also designated SEQ ID:675.

Another function of VGAM340 is therefore inhibition of MSTP043 (Accession NM_{—}031953). Accordingly, utilities of VGAM340 include diagnosis, prevention and treatment of diseases and clinical conditions associated with MSTP043. Nup43 (Accession NM_{—}024647) is another VGAM340 host target gene. Nup43 BINDING SITE is HOST TARGET binding site found in the 3′ untranslated region of mRNA encoded by Nup43, corresponding to a HOST TARGET binding site such as BINDING SITE I, BINDING SITE II or BINDING SITE III. Table 2 illustrates the complementarity of the nucleotide sequences of Nup43 BINDING SITE, designated SEQ ID:2075, to the nucleotide sequence of VGAM340 RNA, herein designated VGAM RNA, also designated SEQ ID:675.

Another function of VGAM340 is therefore inhibition of Nup43 (Accession NM_{—}024647). Accordingly, utilities of VGAM340 include diagnosis, prevention and treatment of diseases and clinical conditions associated with Nup43. Ribosomal Protein S6 Kinase, 90 kDa, Polypeptide 4 (RPS6KA4, Accession NM_{—}003942) is another VGAM340 host target gene. RPS6KA4 BINDING SITE is HOST TARGET binding site found in the 3′ untranslated region of mRNA encoded by RPS6KA4, corresponding to a HOST TARGET binding site such as BINDING SITE I, BINDING SITE II or BINDING SITE III. Table 2 illustrates the complementarity of the nucleotide sequences of RPS6KA4 BINDING SITE, designated SEQ ID: 1072, to the nucleotide sequence of VGAM340 RNA, herein designated VGAM RNA, also designated SEQ ID:675.

Another function of VGAM340 is therefore inhibition of Ribosomal Protein S6 Kinase, 90 kDa, Polypeptide 4 (RPS6KA4, Accession NM_{—}003942). Accordingly, utilities of VGAM340 include diagnosis, prevention and treatment of diseases and clinical conditions associated with RPS6KA4. Solute Carrier Family 17 (sodium-dependent inorganic phosphate cotransporter), Member 6 (SLC17A6, Accession NM_{—}020346) is another VGAM340 host target gene. SLC17A6 BINDING SITE is HOST TARGET binding site found in the 3′ untranslated region of mRNA encoded by SLC17A6, corresponding to a HOST TARGET binding site such as BINDING SITE I, BINDING SITE II or BINDING SITE III. Table 2 illustrates the complementarity of the nucleotide sequences of SLC17A6 BINDING SITE, designated SEQ ID:1911, to the nucleotide sequence of VGAM340 RNA, herein designated VGAM RNA, also designated SEQ ID:675.

Another function of VGAM340 is therefore inhibition of Solute Carrier Family 17 (sodium-dependent inorganic phosphate cotransporter), Member 6 (SLC17A6, Accession NM_{—}020346). Accordingly, utilities of VGAM340 include diagnosis, prevention and treatment of diseases and clinical conditions associated with SLC17A6. TERA (Accession NM_{—}021238) is another VGAM340 host target gene. TERA BINDING SITE is HOST TARGET binding site found in the 3′ untranslated region of mRNA encoded by TERA, corresponding to a HOST TARGET binding site such as BINDING SITE I, BINDING SITE II or BINDING SITE III. Table 2 illustrates the complementarity of the nucleotide sequences of TERA BINDING SITE, designated SEQ ID:1949, to the nucleotide sequence of VGAM340 RNA, herein designated VGAM RNA, also designated SEQ ID:675.

Another function of VGAM340 is therefore inhibition of TERA (Accession NM_{—}021238). Accordingly, utilities of VGAM340 include diagnosis, prevention and treatment of diseases and clinical conditions associated with TERA. Vav 3 Oncogene (VAV3, Accession NM_{—}006113) is another VGAM340 host target gene. VAV3 BINDING SITE is HOST TARGET binding site found in the 3′ untranslated region of mRNA encoded by VAV3, corresponding to a HOST TARGET binding site such as BINDING SITE I, BINDING SITE II or BINDING SITE III. Table 2 illustrates the complementarity of the nucleotide sequences of VAV3 BINDING SITE, designated SEQ ID:1273, to the nucleotide sequence of VGAM340 RNA, herein designated VGAM RNA, also designated SEQ ID:675.

Another function of VGAM340 is therefore inhibition of Vav 3 Oncogene (VAV3, Accession NM_{—}006113). Accordingly, utilities of VGAM340 include diagnosis, prevention and treatment of diseases and clinical conditions associated with VAV3. LOC120892 (Accession XM_{—}058513) is another VGAM340 host target gene. LOC120892 BINDING SITE is HOST TARGET binding site found in the 3′ untranslated region of mRNA encoded by LOC120892, corresponding to a HOST TARGET binding site such as BINDING SITE I, BINDING SITE II or BINDING SITE III. Table 2 illustrates the complementarity of the nucleotide sequences of LOC120892 BINDING SITE, designated SEQ ID:2987, to the nucleotide sequence of VGAM340 RNA, herein designated VGAM RNA, also designated SEQ ID:675.

Another function of VGAM340 is therefore inhibition of LOC120892 (Accession XM_{—}058513). Accordingly, utilities of VGAM340 include diagnosis, prevention and treatment of diseases and clinical conditions associated with LOC120892. LOC155434 (Accession XM_{—}098723) is another VGAM340 host target gene. LOC155434 BINDING SITE is HOST TARGET binding site found in the 3′ untranslated region of mRNA encoded by LOC155434, corresponding to a HOST TARGET binding site such as BINDING SITE I, BINDING SITE II or BINDING SITE III. Table 2 illustrates the complementarity of the nucleotide sequences of LOC155434 BINDING SITE, designated SEQ ID:3376, to the nucleotide sequence of VGAM340 RNA, herein designated VGAM RNA, also designated SEQ ID:675.

Another function of VGAM340 is therefore inhibition of LOC155434 (Accession XM_{—}098723). Accordingly, utilities of VGAM340 include diagnosis, prevention and treatment of diseases and clinical conditions associated with LOC155434. LOC169966 (Accession XM_{—}093010) is another VGAM340 host target gene. LOC169966 BINDING SITE is HOST TARGET binding site found in the 3′ untranslated region of mRNA encoded by LOC169966, corresponding to a HOST TARGET binding site such as BINDING SITE I, BINDING SITE II or BINDING SITE III. Table 2 illustrates the complementarity of the nucleotide sequences of LOC169966 BINDING SITE, designated SEQ ID:3252, to the nucleotide sequence of VGAM340 RNA, herein designated VGAM RNA, also designated SEQ ID:675.

Another function of VGAM340 is therefore inhibition of LOC169966 (Accession XM_{—}093010). Accordingly, utilities of VGAM340 include diagnosis, prevention and treatment of diseases and clinical conditions associated with LOC169966. LOC221272 (Accession XM_{—}168050) is another VGAM340 host target gene. LOC221272 BINDING SITE is HOST TARGET binding site found in the 3′ untranslated region of mRNA encoded by LOC221272, corresponding to a HOST TARGET binding site such as BINDING SITE I, BINDING SITE II or BINDING SITE III. Table 2 illustrates the complementarity of the nucleotide sequences of LOC221272 BINDING SITE, designated SEQ ID:3616, to the nucleotide sequence of VGAM340 RNA, herein designated VGAM RNA, also designated SEQ ID:675.

Another function of VGAM340 is therefore inhibition of LOC221272 (Accession XM_{—}168050). Accordingly, utilities of VGAM340 include diagnosis, prevention and treatment of diseases and clinical conditions associated with LOC221272. LOC221968 (Accession XM_{—}166524) is another VGAM340 host target gene. LOC221968 BINDING SITE is HOST TARGET binding site found in the 3′ untranslated region of mRNA encoded by LOC221968, corresponding to a HOST TARGET binding site such as BINDING SITE I, BINDING SITE II or BINDING SITE III. Table 2 illustrates the complementarity of the nucleotide sequences of LOC221968 BINDING SITE, designated SEQ ID:3569, to the nucleotide sequence of VGAM340 RNA, herein designated VGAM RNA, also designated SEQ ID:675.

Another function of VGAM340 is therefore inhibition of LOC221968 (Accession XM_{—}166524). Accordingly, utilities of VGAM340 include diagnosis, prevention and treatment of diseases and clinical conditions associated with LOC221968. LOC86651 (Accession XM_{—}044052) is another VGAM340 host target gene. LOC86651 BINDING SITE is HOST TARGET binding site found in the 5′ untranslated region of mRNA encoded by LOC86651, corresponding to a HOST TARGET binding site such as BINDING SITE I, BINDING SITE II or BINDING SITE III. Table 2 illustrates the complementarity of the nucleotide sequences of LOC86651 BINDING SITE, designated SEQ ID:2830, to the nucleotide sequence of VGAM340 RNA, herein designated VGAM RNA, also designated SEQ ID:675.

Another function of VGAM340 is therefore inhibition of LOC86651 (Accession XM_{—}044052). Accordingly, utilities of VGAM340 include diagnosis, prevention and treatment of diseases and clinical conditions associated with LOC86651. LOC90322 (Accession XM_{—}030903) is another VGAM340 host target gene. LOC90322 BINDING SITE is HOST TARGET binding site found in the 3′ untranslated region of mRNA encoded by LOC90322, corresponding to a HOST TARGET binding site such as BINDING SITE I, BINDING SITE II or BINDING SITE III. Table 2 illustrates the complementarity of the nucleotide sequences of LOC90322 BINDING SITE, designated SEQ ID:2632, to the nucleotide sequence of VGAM340 RNA, herein designated VGAM RNA, also designated SEQ ID:675.

Another function of VGAM340 is therefore inhibition of LOC90322 (Accession XM_{—}030903). Accordingly, utilities of VGAM340 include diagnosis, prevention and treatment of diseases and clinical conditions associated with LOC90322. FIG. 1 further provides a conceptual description of a novel bioinformatically detected viral gene of the present invention, referred to here as Viral Genomic Address Messenger 341 (VGAM341) viral gene, which modulates expression of respective host target genes thereof, the function and utility of which host target genes is known in the art.

VGAM341 is a novel bioinformatically detected regulatory, non protein coding, viral micro RNA (miRNA) gene. The method by which VGAM341 was detected is described hereinabove with reference to FIGS. 1-8.

VGAM341 gene, herein designated VGAM GENE, is a viral gene contained in the genome of Vaccinia Virus. VGAM341 host target gene, herein designated VGAM HOST TARGET GENE, is a human gene contained in the human genome.

VGAM341 gene encodes a VGAM341 precursor RNA, herein designated VGAM PRECURSOR RNA. Similar to other miRNA genes, and unlike most ordinary genes, VGAM341 precursor RNA does not encode a protein. A nucleotide sequence identical or highly similar to the nucleotide sequence of VGAM341 precursor RNA is designated SEQ ID:327, and is provided hereinbelow with reference to the sequence listing part. Nucleotide sequence SEQ ID:327 is located at position 642 relative to the genome of Vaccinia Virus.

VGAM341 precursor RNA folds onto itself, forming VGAM341 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM341 folded precursor RNA into VGAM341 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM341 RNA is designated SEQ ID:676, and is provided hereinbelow with reference to the sequence listing part.

VGAM341 host target gene, herein designated VGAM HOST TARGET GENE, encodes a corresponding messenger RNA, VGAM341 host target RNA, herein designated VGAM HOST TARGET RNA. VGAM341 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM341 RNA, herein designated VGAM RNA, binds complementarily to one or more host target binding sites located in untranslated regions of VGAM341 host target RNA, herein designated VGAM HOST TARGET RNA. This complementary binding is due to the fact that the nucleotide sequence of VGAM341 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM341 RNA, herein designated VGAM RNA, may have a different number of host target binding sites in untranslated regions of a VGAM341 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM341 RNA, herein designated VGAM RNA, to host target binding sites on VGAM341 host target RNA, herein designated VGAM HOST TARGET RNA, such as BINDING SITE I, BINDING SITE II and BINDING SITE III, inhibits translation of VGAM341 host target RNA into VGAM341 host target protein, herein designated VGAM HOST TARGET PROTEIN. VGAM host target protein is therefore outlined by a broken line.

It is appreciated that VGAM341 host target gene, herein designated VGAM HOST TARGET GENE, in fact represents a plurality of VGAM341 host target genes. The mRNA of each one of this plurality of VGAM341 host target genes comprises one or more host target binding sites, each having a nucleotide sequence which is at least partly complementary to VGAM341 RNA, herein designated VGAM RNA, and which when bound by VGAM341 RNA causes inhibition of translation of respective one or more VGAM341 host target proteins.

It is further appreciated by one skilled in the art that the mode of translational inhibition illustrated by FIG. 1 with specific reference to translational inhibition exerted by VGAM341 gene, herein designated VGAM GENE, on one or more VGAM341 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM341 is inhibition of expression of host target genes, as part of a novel viral mechanism of attacking a host. Accordingly, utilities of VGAM341 include diagnosis, prevention and treatment of viral infection by Vaccinia Virus. Specific functions, and accordingly utilities, of VGAM341 correlate with, and may be deduced from, the identity of the host target genes which VGAM341 binds and inhibits, and the function of these host target genes, as elaborated hereinbelow.

Nucleotide sequences of the VGAM341 precursor RNA, herein designated VGAM PRECURSOR RNA, and of the ‘diced’ VGAM341 RNA, herein designated VGAM RNA, and a schematic representation of the secondary folding of VGAM341 folded precursor RNA, herein designated VGAM FOLDED PRECURSOR RNA, of VGAM341 are further described hereinbelow with reference to Table 1.

Nucleotide sequences of host target binding sites, such as BINDING SITE-I, BINDING SITE-II and BINDING SITE-III of FIG. 1, found on VGAM341 host target RNA, and schematic representation of the complementarity of each of these host target binding sites to VGAM341 RNA, herein designated VGAM RNA, are described hereinbelow with reference to Table 2.

As mentioned hereinabove with reference to FIG. 1, a function of VGAM341 gene, herein designated VGAM is inhibition of expression of VGAM341 target genes. It is appreciated that specific functions, and accordingly utilities, of VGAM341 correlate with, and may be deduced from, the identity of the target genes which VGAM341 binds and inhibits, and the function of these target genes, as elaborated hereinbelow.

Myosin IE (MYO1E, Accession NM_{—}004998) is a VGAM341 host target gene. MYO1E BINDING SITE is HOST TARGET binding site found in the 5′ untranslated region of mRNA encoded by MYO1E, corresponding to a HOST TARGET binding site such as BINDING SITE I, BINDING SITE II or BINDING SITE III. Table 2 illustrates the complementarity of the nucleotide sequences of MYO1E BINDING SITE, designated SEQ ID:1168, to the nucleotide sequence of VGAM341 RNA, herein designated VGAM RNA, also designated SEQ ID:676.

A function of VGAM341 is therefore inhibition of Myosin IE (MYO1E, Accession NM_{—}004998), a gene which is an unconventional myosin. Accordingly, utilities of VGAM341 include diagnosis, prevention and treatment of diseases and clinical conditions associated with MYO1E. The function of MYO1E and its association with various diseases and clinical conditions, has been established by previous studies, as described hereinabove with reference to VGAM35. Rho-associated, Coiled-coil Containing Protein Kinase 2 (ROCK2, Accession XM_{—}038377) is another VGAM341 host target gene. ROCK2 BINDING SITE is HOST TARGET binding site found in the 3′ untranslated region of mRNA encoded by ROCK2, corresponding to a HOST TARGET binding site such as BINDING SITE I, BINDING SITE II or BINDING SITE III. Table 2 illustrates the complementarity of the nucleotide sequences of ROCK2 BINDING SITE, designated SEQ ID:2737, to the nucleotide sequence of VGAM341 RNA, herein designated VGAM RNA, also designated SEQ ID:676.

Another function of VGAM341 is therefore inhibition of Rho-associated, Coiled-coil Containing Protein Kinase 2 (ROCK2, Accession XM_{—}038377), a gene which regulates cytokinesis, smooth muscle contraction, the formation of actin stress fibers and focal adhesions. Accordingly, utilities of VGAM341 include diagnosis, prevention and treatment of diseases and clinical conditions associated with ROCK2. The function of ROCK2 and its association with various diseases and clinical conditions, has been established by previous studies, as described hereinabove with reference to VGAM35. MGC15937 (Accession NM_{—}080661) is another VGAM341 host target gene. MGC15937 BINDING SITE is HOST TARGET binding site found in the 5′ untranslated region of mRNA encoded by MGC15937, corresponding to a HOST TARGET binding site such as BINDING SITE I, BINDING SITE II or BINDING SITE III. Table 2 illustrates the complementarity of the nucleotide sequences of MGC15937 BINDING SITE, designated SEQ ID:2383, to the nucleotide sequence of VGAM341 RNA, herein designated VGAM RNA, also designated SEQ ID:676.

Another function of VGAM341 is therefore inhibition of MGC15937 (Accession NM_{—}080661). Accordingly, utilities of VGAM341 include diagnosis, prevention and treatment of diseases and clinical conditions associated with MGC15937. Serine/threonine Kinase 16 (STK16, Accession XM_{—}050904) is another VGAM341 host target gene. STK16 BINDING SITE is HOST TARGET binding site found in the 5′ untranslated region of mRNA encoded by STK16, corresponding to a HOST TARGET binding site such as BINDING SITE I, BINDING SITE II or BINDING SITE III. Table 2 illustrates the complementarity of the nucleotide sequences of STK16 BINDING SITE, designated SEQ ID:2937, to the nucleotide sequence of VGAM341 RNA, herein designated VGAM RNA, also designated SEQ ID:676.

Another function of VGAM341 is therefore inhibition of Serine/threonine Kinase 16 (STK16, Accession XM_{—}050904). Accordingly, utilities of VGAM341 include diagnosis, prevention and treatment of diseases and clinical conditions associated with STK16. LOC148029 (Accession XM_{—}086014) is another VGAM341 host target gene. LOC148029 BINDING SITE is HOST TARGET binding site found in the 5′ untranslated region of mRNA encoded by LOC148029, corresponding to a HOST TARGET binding site such as BINDING SITE I, BINDING SITE II or BINDING SITE III. Table 2 illustrates the complementarity of the nucleotide sequences of LOC148029 BINDING SITE, designated SEQ ID:3116, to the nucleotide sequence of VGAM341 RNA, herein designated VGAM RNA, also designated SEQ ID:676.

Another function of VGAM341 is therefore inhibition of LOC148029 (Accession XM_{—}086014). Accordingly, utilities of VGAM341 include diagnosis, prevention and treatment of diseases and clinical conditions associated with LOC148029. LOC169436 (Accession XM_{—}095696) is another VGAM341 host target gene. LOC169436 BINDING SITE is HOST TARGET binding site found in the 5′ untranslated region of mRNA encoded by LOC169436, corresponding to a HOST TARGET binding site such as BINDING SITE I, BINDING SITE II or BINDING SITE III. Table 2 illustrates the complementarity of the nucleotide sequences of LOC169436 BINDING SITE, designated SEQ ID:3261, to the nucleotide sequence of VGAM341 RNA, herein designated VGAM RNA, also designated SEQ ID:676.

Another function of VGAM341 is therefore inhibition of LOC169436 (Accession XM_{—}095696). Accordingly, utilities of VGAM341 include diagnosis, prevention and treatment of diseases and clinical conditions associated with LOC169436. LOC256267 (Accession XM_{—}173007) is another VGAM341 host target gene. LOC256267 BINDING SITE is HOST TARGET binding site found in the 3′ untranslated region of mRNA encoded by LOC256267, corresponding to a HOST TARGET binding site such as BINDING SITE I, BINDING SITE II or BINDING SITE III. Table 2 illustrates the complementarity of the nucleotide sequences of LOC256267 BINDING SITE, designated SEQ ID:3713, to the nucleotide sequence of VGAM341 RNA, herein designated VGAM RNA, also designated SEQ ID:676.

Another function of VGAM341 is therefore inhibition of LOC256267 (Accession XM_{—}173007). Accordingly, utilities of VGAM341 include diagnosis, prevention and treatment of diseases and clinical conditions associated with LOC256267. LOC257426 (Accession XM_{—}039451) is another VGAM341 host target gene. LOC257426 BINDING SITE is HOST TARGET binding site found in the 3′ untranslated region of mRNA encoded by LOC257426, corresponding to a HOST TARGET binding site such as BINDING SITE I, BINDING SITE II or BINDING SITE III. Table 2 illustrates the complementarity of the nucleotide sequences of LOC257426 BINDING SITE, designated SEQ ID:2755, to the nucleotide sequence of VGAM341 RNA, herein designated VGAM RNA, also designated SEQ ID:676.

Another function of VGAM341 is therefore inhibition of LOC257426 (Accession XM_{—}039451). Accordingly, utilities of VGAM341 include diagnosis, prevention and treatment of diseases and clinical conditions associated with LOC257426. LOC92466 (Accession XM_{—}045251) is another VGAM341 host target gene. LOC92466 BINDING SITE is HOST TARGET binding site found in the 3′ untranslated region of mRNA encoded by LOC92466, corresponding to a HOST TARGET binding site such as BINDING SITE I, BINDING SITE II or BINDING SITE III. Table 2 illustrates the complementarity of the nucleotide sequences of LOC92466 BINDING SITE, designated SEQ ID:2858, to the nucleotide sequence of VGAM341 RNA, herein designated VGAM RNA, also designated SEQ ID:676.

Another function of VGAM341 is therefore inhibition of LOC92466 (Accession XM_{—}045251). Accordingly, utilities of VGAM341 include diagnosis, prevention and treatment of diseases and clinical conditions associated with LOC92466. FIG. 1 further provides a conceptual description of a novel bioinformatically detected viral gene of the present invention, referred to here as Viral Genomic Address Messenger 342 (VGAM342) viral gene, which modulates expression of respective host target genes thereof, the function and utility of which host target genes is known in the art.

VGAM342 is a novel bioinformatically detected regulatory, non protein coding, viral micro RNA (miRNA) gene. The method by which VGAM342 was detected is described hereinabove with reference to FIGS. 1-8.

VGAM342 gene, herein designated VGAM GENE, is a viral gene contained in the genome of Vaccinia Virus. VGAM342 host target gene, herein designated VGAM HOST TARGET GENE, is a human gene contained in the human genome.

VGAM342 gene encodes a VGAM342 precursor RNA, herein designated VGAM PRECURSOR RNA. Similar to other miRNA genes, and unlike most ordinary genes, VGAM342 precursor RNA does not encode a protein. A nucleotide sequence identical or highly similar to the nucleotide sequence of VGAM342 precursor RNA is designated SEQ ID:328, and is provided hereinbelow with reference to the sequence listing part. Nucleotide sequence SEQ ID:328 is located at position 3549 relative to the genome of Vaccinia Virus.

VGAM342 precursor RNA folds onto itself, forming VGAM342 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM342 folded precursor RNA into VGAM342 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM342 RNA is designated SEQ ID:677, and is provided hereinbelow with reference to the sequence listing part.

VGAM342 host target gene, herein designated VGAM HOST TARGET GENE, encodes a corresponding messenger RNA, VGAM342 host target RNA, herein designated VGAM HOST TARGET RNA. VGAM342 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM342 RNA, herein designated VGAM RNA, binds complementarily to one or more host target binding sites located in untranslated regions of VGAM342 host target RNA, herein designated VGAM HOST TARGET RNA. This complementary binding is due to the fact that the nucleotide sequence of VGAM342 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM342 RNA, herein designated VGAM RNA, may have a different number of host target binding sites in untranslated regions of a VGAM342 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM342 RNA, herein designated VGAM RNA, to host target binding sites on VGAM342 host target RNA, herein designated VGAM HOST TARGET RNA, such as BINDING SITE I, BINDING SITE II and BINDING SITE III, inhibits translation of VGAM342 host target RNA into VGAM342 host target protein, herein designated VGAM HOST TARGET PROTEIN. VGAM host target protein is therefore outlined by a broken line.

It is appreciated that VGAM342 host target gene, herein designated VGAM HOST TARGET GENE, in fact represents a plurality of VGAM342 host target genes. The mRNA of each one of this plurality of VGAM342 host target genes comprises one or more host target binding sites, each having a nucleotide sequence which is at least partly complementary to VGAM342 RNA, herein designated VGAM RNA, and which when bound by VGAM342 RNA causes inhibition of translation of respective one or more VGAM342 host target proteins.

It is further appreciated by one skilled in the art that the mode of translational inhibition illustrated by FIG. 1 with specific reference to translational inhibition exerted by VGAM342 gene, herein designated VGAM GENE, on one or more VGAM342 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM342 is inhibition of expression of host target genes, as part of a novel viral mechanism of attacking a host. Accordingly, utilities of VGAM342 include diagnosis, prevention and treatment of viral infection by Vaccinia Virus. Specific functions, and accordingly utilities, of VGAM342 correlate with, and may be deduced from, the identity of the host target genes which VGAM342 binds and inhibits, and the function of these host target genes, as elaborated hereinbelow.

Nucleotide sequences of the VGAM342 precursor RNA, herein designated VGAM PRECURSOR RNA, and of the ‘diced’ VGAM342 RNA, herein designated VGAM RNA, and a schematic representation of the secondary folding of VGAM342 folded precursor RNA, herein designated VGAM FOLDED PRECURSOR RNA, of VGAM342 are further described hereinbelow with reference to Table 1.

Nucleotide sequences of host target binding sites, such as BINDING SITE-I, BINDING SITE-II and BINDING SITE-III of FIG. 1, found on VGAM342 host target RNA, and schematic representation of the complementarity of each of these host target binding sites to VGAM342 RNA, herein designated VGAM RNA, are described hereinbelow with reference to Table 2.

As mentioned hereinabove with reference to FIG. 1, a function of VGAM342 gene, herein designated VGAM is inhibition of expression of VGAM342 target genes. It is appreciated that specific functions, and accordingly utilities, of VGAM342 correlate with, and may be deduced from, the identity of the target genes which VGAM342 binds and inhibits, and the function of these target genes, as elaborated hereinbelow.

Membrane Metallo-endopeptidase (neutral endopeptidase, enkephalinase, CALLA, CD10) (MME, Accession NM_{—}000902) is a VGAM342 host target gene. MME BINDING SITE1 through MME BINDING SITE4 are HOST TARGET binding sites found in untranslated regions of mRNA encoded by MME, corresponding to HOST TARGET binding sites such as BINDING SITE I, BINDING SITE II or BINDING SITE III. Table 2 illustrates the complementarity of the nucleotide sequences of MME BINDING SITE1 through MME BINDING SITE4, designated SEQ ID:785, SEQ ID:1377, SEQ ID:1378 and SEQ ID:1379 respectively, to the nucleotide sequence of VGAM342 RNA, herein designated VGAM RNA, also designated SEQ ID:677.

A function of VGAM342 is therefore inhibition of Membrane Metallo-endopeptidase (neutral endopeptidase, enkephalinase, CALLA, CD10) (MME, Accession NM_{—}000902), a gene which is thermolysin-like specificity. Accordingly, utilities of VGAM342 include diagnosis, prevention and treatment of diseases and clinical conditions associated with MME. The function of MME and its association with various diseases and clinical conditions, has been established by previous studies, as described hereinabove with reference to VGAM36. FLJ11275 (Accession NM_{—}018376) is another VGAM342 host target gene. FLJ11275 BINDING SITE is HOST TARGET binding site found in the 3′ untranslated region of mRNA encoded by FLJ11275, corresponding to a HOST TARGET binding site such as BINDING SITE I, BINDING SITE II or BINDING SITE III. Table 2 illustrates the complementarity of the nucleotide sequences of FLJ11275 BINDING SITE, designated SEQ ID:1821, to the nucleotide sequence of VGAM342 RNA, herein designated VGAM RNA, also designated SEQ ID:677.

Another function of VGAM342 is therefore inhibition of FLJ11275 (Accession NM_{—}018376). Accordingly, utilities of VGAM342 include diagnosis, prevention and treatment of diseases and clinical conditions associated with FLJ11275. Golgi Phosphoprotein 3 (coat-protein) (GOLPH3, Accession NM_{—}022130) is another VGAM342 host target gene. GOLPH3 BINDING SITE is HOST TARGET binding site found in the 3′ untranslated region of mRNA encoded by GOLPH3, corresponding to a HOST TARGET binding site such as BINDING SITE I, BINDING SITE II or BINDING SITE III. Table 2 illustrates the complementarity of the nucleotide sequences of GOLPH3 BINDING SITE, designated SEQ ID:1981, to the nucleotide sequence of VGAM342 RNA, herein designated VGAM RNA, also designated SEQ ID:677.

Another function of VGAM342 is therefore inhibition of Golgi Phosphoprotein 3 (coat-protein) (GOLPH3, Accession NM_{—}022130). Accordingly, utilities of VGAM342 include diagnosis, prevention and treatment of diseases and clinical conditions associated with GOLPH3. KIAA0563 (Accession NM_{—}014834) is another VGAM342 host target gene. KIAA0563 BINDING SITE is HOST TARGET binding site found in the 5′ untranslated region of mRNA encoded by KIAA0563, corresponding to a HOST TARGET binding site such as BINDING SITE I, BINDING SITE II or BINDING SITE III. Table 2 illustrates the complementarity of the nucleotide sequences of KIAA0563 BINDING SITE, designated SEQ ID: 1565, to the nucleotide sequence of VGAM342 RNA, herein designated VGAM RNA, also designated SEQ ID:677.

Another function of VGAM342 is therefore inhibition of KIAA0563 (Accession NM_{—}014834). Accordingly, utilities of VGAM342 include diagnosis, prevention and treatment of diseases and clinical conditions associated with KIAA0563. TAF2 RNA Polymerase II, TATA Box Binding Protein (TBP)-associated Factor, 150 kDa (TAF2, Accession NM_{—}003184) is another VGAM342 host target gene. TAF2 BINDING SITE is HOST TARGET binding site found in the 3′ untranslated region of mRNA encoded by TAF2, corresponding to a HOST TARGET binding site such as BINDING SITE I, BINDING SITE II or BINDING SITE III. Table 2 illustrates the complementarity of the nucleotide sequences of TAF2 BINDING SITE, designated SEQ ID:997, to the nucleotide sequence of VGAM342 RNA, herein designated VGAM RNA, also designated SEQ ID:677.

Another function of VGAM342 is therefore inhibition of TAF2 RNA Polymerase II, TATA Box Binding Protein (TBP)-associated Factor, 150 kDa (TAF2, Accession NM_{—}003184). Accordingly, utilities of VGAM342 include diagnosis, prevention and treatment of diseases and clinical conditions associated with TAF2. LOC147071 (Accession XM_{—}054031) is another VGAM342 host target gene. LOC147071 BINDING SITE is HOST TARGET binding site found in the 5′ untranslated region of mRNA encoded by LOC147071, corresponding to a HOST TARGET binding site such as BINDING SITE I, BINDING SITE II or BINDING SITE III. Table 2 illustrates the complementarity of the nucleotide sequences of LOC147071 BINDING SITE, designated SEQ ID:2965, to the nucleotide sequence of VGAM342 RNA, herein designated VGAM RNA, also designated SEQ ID:677.

Another function of VGAM342 is therefore inhibition of LOC147071 (Accession XM_{—}054031). Accordingly, utilities of VGAM342 include diagnosis, prevention and treatment of diseases and clinical conditions associated with LOC147071. LOC147639 (Accession XM_{—}085822) is another VGAM342 host target gene. LOC147639 BINDING SITE is HOST TARGET binding site found in the 5′ untranslated region of mRNA encoded by LOC147639, corresponding to a HOST TARGET binding site such as BINDING SITE I, BINDING SITE II or BINDING SITE III. Table 2 illustrates the complementarity of the nucleotide sequences of LOC147639 BINDING SITE, designated SEQ ID:3110, to the nucleotide sequence of VGAM342 RNA, herein designated VGAM RNA, also designated SEQ ID:677.

Another function of VGAM342 is therefore inhibition of LOC147639 (Accession XM_{—}085822). Accordingly, utilities of VGAM342 include diagnosis, prevention and treatment of diseases and clinical conditions associated with LOC147639. LOC201173 (Accession XM_{—}113312) is another VGAM342 host target gene. LOC201173 BINDING SITE is HOST TARGET binding site found in the 5′ untranslated region of mRNA encoded by LOC201173, corresponding to a HOST TARGET binding site such as BINDING SITE I, BINDING SITE II or BINDING SITE III. Table 2 illustrates the complementarity of the nucleotide sequences of LOC201173 BINDING SITE, designated SEQ ID:3405, to the nucleotide sequence of VGAM342 RNA, herein designated VGAM RNA, also designated SEQ ID:677.

Another function of VGAM342 is therefore inhibition of LOC201173 (Accession XM_{—}113312). Accordingly, utilities of VGAM342 include diagnosis, prevention and treatment of diseases and clinical conditions associated with LOC201173. LOC201220 (Accession XM_{—}113321) is another VGAM342 host target gene. LOC201220 BINDING SITE is HOST TARGET binding site found in the 5′ untranslated region of mRNA encoded by LOC201220, corresponding to a HOST TARGET binding site such as BINDING SITE I, BINDING SITE II or BINDING SITE III. Table 2 illustrates the complementarity of the nucleotide sequences of LOC201220 BINDING SITE, designated SEQ ID:3406, to the nucleotide sequence of VGAM342 RNA, herein designated VGAM RNA, also designated SEQ ID:677.

Another function of VGAM342 is therefore inhibition of LOC201220 (Accession XM_{—}113321). Accordingly, utilities of VGAM342 include diagnosis, prevention and treatment of diseases and clinical conditions associated with LOC201220. FIG. 1 further provides a conceptual description of a novel bioinformatically detected viral gene of the present invention, referred to here as Viral Genomic Address Messenger 343 (VGAM343) viral gene, which modulates expression of respective host target genes thereof, the function and utility of which host target genes is known in the art.

VGAM343 is a novel bioinformatically detected regulatory, non protein coding, viral micro RNA (miRNA) gene. The method by which VGAM343 was detected is described hereinabove with reference to FIGS. 1-8.

VGAM343 gene, herein designated VGAM GENE, is a viral gene contained in the genome of Vaccinia Virus. VGAM343 host target gene, herein designated VGAM HOST TARGET GENE, is a human gene contained in the human genome.

VGAM343 gene encodes a VGAM343 precursor RNA, herein designated VGAM PRECURSOR RNA. Similar to other miRNA genes, and unlike most ordinary genes, VGAM343 precursor RNA does not encode a protein. A nucleotide sequence identical or highly similar to the nucleotide sequence of VGAM343 precursor RNA is designated SEQ ID:329, and is provided hereinbelow with reference to the sequence listing part. Nucleotide sequence SEQ ID:329 is located at position 3549 relative to the genome of Vaccinia Virus.

VGAM343 precursor RNA folds onto itself, forming VGAM343 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM343 folded precursor RNA into VGAM343 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM343 RNA is designated SEQ ID:678, and is provided hereinbelow with reference to the sequence listing part.

VGAM343 host target gene, herein designated VGAM HOST TARGET GENE, encodes a corresponding messenger RNA, VGAM343 host target RNA, herein designated VGAM HOST TARGET RNA. VGAM343 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM343 RNA, herein designated VGAM RNA, binds complementarily to one or more host target binding sites located in untranslated regions of VGAM343 host target RNA, herein designated VGAM HOST TARGET RNA. This complementary binding is due to the fact that the nucleotide sequence of VGAM343 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM343 RNA, herein designated VGAM RNA, may have a different number of host target binding sites in untranslated regions of a VGAM343 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM343 RNA, herein designated VGAM RNA, to host target binding sites on VGAM343 host target RNA, herein designated VGAM HOST TARGET RNA, such as BINDING SITE I, BINDING SITE II and BINDING SITE III, inhibits translation of VGAM343 host target RNA into VGAM343 host target protein, herein designated VGAM HOST TARGET PROTEIN. VGAM host target protein is therefore outlined by a broken line.

It is appreciated that VGAM343 host target gene, herein designated VGAM HOST TARGET GENE, in fact represents a plurality of VGAM343 host target genes. The mRNA of each one of this plurality of VGAM343 host target genes comprises one or more host target binding sites, each having a nucleotide sequence which is at least partly complementary to VGAM343 RNA, herein designated VGAM RNA, and which when bound by VGAM343 RNA causes inhibition of translation of respective one or more VGAM343 host target proteins.

It is further appreciated by one skilled in the art that the mode of translational inhibition illustrated by FIG. 1 with specific reference to translational inhibition exerted by VGAM343 gene, herein designated VGAM GENE, on one or more VGAM343 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM343 is inhibition of expression of host target genes, as part of a novel viral mechanism of attacking a host. Accordingly, utilities of VGAM343 include diagnosis, prevention and treatment of viral infection by Vaccinia Virus. Specific functions, and accordingly utilities, of VGAM343 correlate with, and may be deduced from, the identity of the host target genes which VGAM343 binds and inhibits, and the function of these host target genes, as elaborated hereinbelow.

Nucleotide sequences of the VGAM343 precursor RNA, herein designated VGAM PRECURSOR RNA, and of the ‘diced’ VGAM343 RNA, herein designated VGAM RNA, and a schematic representation of the secondary folding of VGAM343 folded precursor RNA, herein designated VGAM FOLDED PRECURSOR RNA, of VGAM343 are further described hereinbelow with reference to Table 1.

Nucleotide sequences of host target binding sites, such as BINDING SITE-I, BINDING SITE-II and BINDING SITE-III of FIG. 1, found on VGAM343 host target RNA, and schematic representation of the complementarity of each of these host target binding sites to VGAM343 RNA, herein designated VGAM RNA, are described hereinbelow with reference to Table 2.

As mentioned hereinabove with reference to FIG. 1, a function of VGAM343 gene, herein designated VGAM is inhibition of expression of VGAM343 target genes. It is appreciated that specific functions, and accordingly utilities, of VGAM343 correlate with, and may be deduced from, the identity of the target genes which VGAM343 binds and inhibits, and the function of these target genes, as elaborated hereinbelow.

Membrane Metallo-endopeptidase (neutral endopeptidase, enkephalinase, CALLA, CD10) (MME, Accession NM_{—}000902) is a VGAM343 host target gene. MME BINDING SITE1 through MME BINDING SITE4 are HOST TARGET binding sites found in untranslated regions of mRNA encoded by MME, corresponding to HOST TARGET binding sites such as BINDING SITE I, BINDING SITE II or BINDING SITE III. Table 2 illustrates the complementarity of the nucleotide sequences of MME BINDING SITE1 through MME BINDING SITE4, designated SEQ ID:785, SEQ ID:1377, SEQ ID:1378 and SEQ ID:1379 respectively, to the nucleotide sequence of VGAM343 RNA, herein designated VGAM RNA, also designated SEQ ID:678.

A function of VGAM343 is therefore inhibition of Membrane Metallo-endopeptidase (neutral endopeptidase, enkephalinase, CALLA, CD10) (MME, Accession NM_{—}000902), a gene which is thermolysin-like specificity. Accordingly, utilities of VGAM343 include diagnosis, prevention and treatment of diseases and clinical conditions associated with MME. The function of MME and its association with various diseases and clinical conditions, has been established by previous studies, as described hereinabove with reference to VGAM36. FLJ11275 (Accession NM_{—}018376) is another VGAM343 host target gene. FLJ11275 BINDING SITE is HOST TARGET binding site found in the 3′ untranslated region of mRNA encoded by FLJ11275, corresponding to a HOST TARGET binding site such as BINDING SITE I, BINDING SITE II or BINDING SITE III. Table 2 illustrates the complementarity of the nucleotide sequences of FLJ11275 BINDING SITE, designated SEQ ID:1821, to the nucleotide sequence of VGAM343 RNA, herein designated VGAM RNA, also designated SEQ ID:678.

Another function of VGAM343 is therefore inhibition of FLJ11275 (Accession NM_{—}018376). Accordingly, utilities of VGAM343 include diagnosis, prevention and treatment of diseases and clinical conditions associated with FLJ11275. Golgi Phosphoprotein 3 (coat-protein) (GOLPH3, Accession NM_{—}022130) is another VGAM343 host target gene. GOLPH3 BINDING SITE is HOST TARGET binding site found in the 3′ untranslated region of mRNA encoded by GOLPH3, corresponding to a HOST TARGET binding site such as BINDING SITE I, BINDING SITE II or BINDING SITE III. Table 2 illustrates the complementarity of the nucleotide sequences of GOLPH3 BINDING SITE, designated SEQ ID:1981, to the nucleotide sequence of VGAM343 RNA, herein designated VGAM RNA, also designated SEQ ID:678.

Another function of VGAM343 is therefore inhibition of Golgi Phosphoprotein 3 (coat-protein) (GOLPH3, Accession NM_{—}022130). Accordingly, utilities of VGAM343 include diagnosis, prevention and treatment of diseases and clinical conditions associated with GOLPH3. KIAA0563 (Accession NM_{—}014834) is another VGAM343 host target gene. KIAA0563 BINDING SITE is HOST TARGET binding site found in the 5′ untranslated region of mRNA encoded by KIAA0563, corresponding to a HOST TARGET binding site such as BINDING SITE I, BINDING SITE II or BINDING SITE III. Table 2 illustrates the complementarity of the nucleotide sequences of KIAA0563 BINDING SITE, designated SEQ ID: 1565, to the nucleotide sequence of VGAM343 RNA, herein designated VGAM RNA, also designated SEQ ID:678.

Another function of VGAM343 is therefore inhibition of KIAA0563 (Accession NM_{—}014834). Accordingly, utilities of VGAM343 include diagnosis, prevention and treatment of diseases and clinical conditions associated with KIAA0563. TAF2 RNA Polymerase II, TATA Box Binding Protein (TBP)-associated Factor, 150 kDa (TAF2, Accession NM_{—}003184) is another VGAM343 host target gene. TAF2 BINDING SITE is HOST TARGET binding site found in the 3′ untranslated region of mRNA encoded by TAF2, corresponding to a HOST TARGET binding site such as BINDING SITE I, BINDING SITE II or BINDING SITE III. Table 2 illustrates the complementarity of the nucleotide sequences of TAF2 BINDING SITE, designated SEQ ID:997, to the nucleotide sequence of VGAM343 RNA, herein designated VGAM RNA, also designated SEQ ID:678.

Another function of VGAM343 is therefore inhibition of TAF2 RNA Polymerase II, TATA Box Binding Protein (TBP)-associated Factor, 150 kDa (TAF2, Accession NM_{—}003184). Accordingly, utilities of VGAM343 include diagnosis, prevention and treatment of diseases and clinical conditions associated with TAF2. LOC147071 (Accession XM_{—}054031) is another VGAM343 host target gene. LOC147071 BINDING SITE is HOST TARGET binding site found in the 5′ untranslated region of mRNA encoded by LOC147071, corresponding to a HOST TARGET binding site such as BINDING SITE I, BINDING SITE II or BINDING SITE III. Table 2 illustrates the complementarity of the nucleotide sequences of LOC147071 BINDING SITE, designated SEQ ID:2965, to the nucleotide sequence of VGAM343 RNA, herein designated VGAM RNA, also designated SEQ ID:678.

Another function of VGAM343 is therefore inhibition of LOC147071 (Accession XM_{—}054031). Accordingly, utilities of VGAM343 include diagnosis, prevention and treatment of diseases and clinical conditions associated with LOC147071. LOC147639 (Accession XM_{—}085822) is another VGAM343 host target gene. LOC147639 BINDING SITE is HOST TARGET binding site found in the 5′ untranslated region of mRNA encoded by LOC147639, corresponding to a HOST TARGET binding site such as BINDING SITE I, BINDING SITE II or BINDING SITE III. Table 2 illustrates the complementarity of the nucleotide sequences of LOC147639 BINDING SITE, designated SEQ ID:3110, to the nucleotide sequence of VGAM343 RNA, herein designated VGAM RNA, also designated SEQ ID:678.

Another function of VGAM343 is therefore inhibition of LOC147639 (Accession XM_{—}085822). Accordingly, utilities of VGAM343 include diagnosis, prevention and treatment of diseases and clinical conditions associated with LOC147639. LOC201173 (Accession XM_{—}113312) is another VGAM343 host target gene. LOC201173 BINDING SITE is HOST TARGET binding site found in the 5′ untranslated region of mRNA encoded by LOC201173, corresponding to a HOST TARGET binding site such as BINDING SITE I, BINDING SITE II or BINDING SITE III. Table 2 illustrates the complementarity of the nucleotide sequences of LOC201173 BINDING SITE, designated SEQ ID:3405, to the nucleotide sequence of VGAM343 RNA, herein designated VGAM RNA, also designated SEQ ID:678.

Another function of VGAM343 is therefore inhibition of LOC201173 (Accession XM_{—}113312). Accordingly, utilities of VGAM343 include diagnosis, prevention and treatment of diseases and clinical conditions associated with LOC201173. LOC201220 (Accession XM_{—}113321) is another VGAM343 host target gene. LOC201220 BINDING SITE is HOST TARGET binding site found in the 5′ untranslated region of mRNA encoded by LOC201220, corresponding to a HOST TARGET binding site such as BINDING SITE I, BINDING SITE II or BINDING SITE III. Table 2 illustrates the complementarity of the nucleotide sequences of LOC201220 BINDING SITE, designated SEQ ID:3406, to the nucleotide sequence of VGAM343 RNA, herein designated VGAM RNA, also designated SEQ ID:678.

Another function of VGAM343 is therefore inhibition of LOC201220 (Accession XM_{—}113321). Accordingly, utilities of VGAM343 include diagnosis, prevention and treatment of diseases and clinical conditions associated with LOC201220. FIG. 1 further provides a conceptual description of a novel bioinformatically detected viral gene of the present invention, referred to here as Viral Genomic Address Messenger 344 (VGAM344) viral gene, which modulates expression of respective host target genes thereof, the function and utility of which host target genes is known in the art.

VGAM344 is a novel bioinformatically detected regulatory, non protein coding, viral micro RNA (miRNA) gene. The method by which VGAM344 was detected is described hereinabove with reference to FIGS. 1-8.

VGAM344 gene, herein designated VGAM GENE, is a viral gene contained in the genome of Vaccinia Virus. VGAM344 host target gene, herein designated VGAM HOST TARGET GENE, is a human gene contained in the human genome.

VGAM344 gene encodes a VGAM344 precursor RNA, herein designated VGAM PRECURSOR RNA. Similar to other miRNA genes, and unlike most ordinary genes, VGAM344 precursor RNA does not encode a protein. A nucleotide sequence identical or highly similar to the nucleotide sequence of VGAM344 precursor RNA is designated SEQ ID:330, and is provided hereinbelow with reference to the sequence listing part. Nucleotide sequence SEQ ID:330 is located at position 2981 relative to the genome of Vaccinia Virus.

VGAM344 precursor RNA folds onto itself, forming VGAM344 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM344 folded precursor RNA into VGAM344 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 79%) nucleotide sequence of VGAM344 RNA is designated SEQ ID:679, and is provided hereinbelow with reference to the sequence listing part.

VGAM344 host target gene, herein designated VGAM HOST TARGET GENE, encodes a corresponding messenger RNA, VGAM344 host target RNA, herein designated VGAM HOST TARGET RNA. VGAM344 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM344 RNA, herein designated VGAM RNA, binds complementarily to one or more host target binding sites located in untranslated regions of VGAM344 host target RNA, herein designated VGAM HOST TARGET RNA. This complementary binding is due to the fact that the nucleotide sequence of VGAM344 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM344 RNA, herein designated VGAM RNA, may have a different number of host target binding sites in untranslated regions of a VGAM344 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM344 RNA, herein designated VGAM RNA, to host target binding sites on VGAM344 host target RNA, herein designated VGAM HOST TARGET RNA, such as BINDING SITE I, BINDING SITE II and BINDING SITE III, inhibits translation of VGAM344 host target RNA into VGAM344 host target protein, herein designated VGAM HOST TARGET PROTEIN. VGAM host target protein is therefore outlined by a broken line.

It is appreciated that VGAM344 host target gene, herein designated VGAM HOST TARGET GENE, in fact represents a plurality of VGAM344 host target genes. The mRNA of each one of this plurality of VGAM344 host target genes comprises one or more host target binding sites, each having a nucleotide sequence which is at least partly complementary to VGAM344 RNA, herein designated VGAM RNA, and which when bound by VGAM344 RNA causes inhibition of translation of respective one or more VGAM344 host target proteins.

It is further appreciated by one skilled in the art that the mode of translational inhibition illustrated by FIG. 1 with specific reference to translational inhibition exerted by VGAM344 gene, herein designated VGAM GENE, on one or more VGAM344 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM344 is inhibition of expression of host target genes, as part of a novel viral mechanism of attacking a host. Accordingly, utilities of VGAM344 include diagnosis, prevention and treatment of viral infection by Vaccinia Virus. Specific functions, and accordingly utilities, of VGAM344 correlate with, and may be deduced from, the identity of the host target genes which VGAM344 binds and inhibits, and the function of these host target genes, as elaborated hereinbelow.

Nucleotide sequences of the VGAM344 precursor RNA, herein designated VGAM PRECURSOR RNA, and of the ‘diced’ VGAM344 RNA, herein designated VGAM RNA, and a schematic representation of the secondary folding of VGAM344 folded precursor RNA, herein designated VGAM FOLDED PRECURSOR RNA, of VGAM344 are further described hereinbelow with reference to Table 1.

Nucleotide sequences of host target binding sites, such as BINDING SITE-I, BINDING SITE-II and BINDING SITE-III of FIG. 1, found on VGAM344 host target RNA, and schematic representation of the complementarity of each of these host target binding sites to VGAM344 RNA, herein designated VGAM RNA, are described hereinbelow with reference to Table 2.

As mentioned hereinabove with reference to FIG. 1, a function of VGAM344 gene, herein designated VGAM is inhibition of expression of VGAM344 target genes. It is appreciated that specific functions, and accordingly utilities, of VGAM344 correlate with, and may be deduced from, the identity of the target genes which VGAM344 binds and inhibits, and the function of these target genes, as elaborated hereinbelow.

MAP/microtubule Affinity-regulating Kinase 1 (MARK1, Accession NM_{—}018650) is a VGAM344 host target gene. MARK1 BINDING SITE is HOST TARGET binding site found in the 3′ untranslated region of mRNA encoded by MARK1, corresponding to a HOST TARGET binding site such as BINDING SITE I, BINDING SITE II or BINDING SITE III. Table 2 illustrates the complementarity of the nucleotide sequences of MARK1 BINDING SITE, designated SEQ ID:1857, to the nucleotide sequence of VGAM344 RNA, herein designated VGAM RNA, also designated SEQ ID:679.

A function of VGAM344 is therefore inhibition of MAP/microtubule Affinity-regulating Kinase 1 (MARK1, Accession NM_{—}018650), a gene which phosphorylate microtubule-associated proteins and trigger microtubule disruption. Accordingly, utilities of VGAM344 include diagnosis, prevention and treatment of diseases and clinical conditions associated with MARK1. The function of MARK1 and its association with various diseases and clinical conditions, has been established by previous studies, as described hereinabove with reference to VGAM38. FIG. 1 further provides a conceptual description of a novel bioinformatically detected viral gene of the present invention, referred to here as Viral Genomic Address Messenger 345 (VGAM345) viral gene, which modulates expression of respective host target genes thereof, the function and utility of which host target genes is known in the art.

VGAM345 is a novel bioinformatically detected regulatory, non protein coding, viral micro RNA (miRNA) gene. The method by which VGAM345 was detected is described hereinabove with reference to FIGS. 1-8.

VGAM345 gene, herein designated VGAM GENE, is a viral gene contained in the genome of Vaccinia Virus. VGAM345 host target gene, herein designated VGAM HOST TARGET GENE, is a human gene contained in the human genome.

VGAM345 gene encodes a VGAM345 precursor RNA, herein designated VGAM PRECURSOR RNA. Similar to other miRNA genes, and unlike most ordinary genes, VGAM345 precursor RNA does not encode a protein. A nucleotide sequence identical or highly similar to the nucleotide sequence of VGAM345 precursor RNA is designated SEQ ID:331, and is provided hereinbelow with reference to the sequence listing part. Nucleotide sequence SEQ ID:331 is located at position 963 relative to the genome of Vaccinia Virus.

VGAM345 precursor RNA folds onto itself, forming VGAM345 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM345 folded precursor RNA into VGAM345 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM345 RNA is designated SEQ ID:680, and is provided hereinbelow with reference to the sequence listing part.

VGAM345 host target gene, herein designated VGAM HOST TARGET GENE, encodes a corresponding messenger RNA, VGAM345 host target RNA, herein designated VGAM HOST TARGET RNA. VGAM345 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM345 RNA, herein designated VGAM RNA, binds complementarily to one or more host target binding sites located in untranslated regions of VGAM345 host target RNA, herein designated VGAM HOST TARGET RNA. This complementary binding is due to the fact that the nucleotide sequence of VGAM345 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM345 RNA, herein designated VGAM RNA, may have a different number of host target binding sites in untranslated regions of a VGAM345 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM345 RNA, herein designated VGAM RNA, to host target binding sites on VGAM345 host target RNA, herein designated VGAM HOST TARGET RNA, such as BINDING SITE I, BINDING SITE II and BINDING SITE III, inhibits translation of VGAM345 host target RNA into VGAM345 host target protein, herein designated VGAM HOST TARGET PROTEIN. VGAM host target protein is therefore outlined by a broken line.

It is appreciated that VGAM345 host target gene, herein designated VGAM HOST TARGET GENE, in fact represents a plurality of VGAM345 host target genes. The mRNA of each one of this plurality of VGAM345 host target genes comprises one or more host target binding sites, each having a nucleotide sequence which is at least partly complementary to VGAM345 RNA, herein designated VGAM RNA, and which when bound by VGAM345 RNA causes inhibition of translation of respective one or more VGAM345 host target proteins.

It is further appreciated by one skilled in the art that the mode of translational inhibition illustrated by FIG. 1 with specific reference to translational inhibition exerted by VGAM345 gene, herein designated VGAM GENE, on one or more VGAM345 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM345 is inhibition of expression of host target genes, as part of a novel viral mechanism of attacking a host. Accordingly, utilities of VGAM345 include diagnosis, prevention and treatment of viral infection by Vaccinia Virus. Specific functions, and accordingly utilities, of VGAM345 correlate with, and may be deduced from, the identity of the host target genes which VGAM345 binds and inhibits, and the function of these host target genes, as elaborated hereinbelow.

Nucleotide sequences of the VGAM345 precursor RNA, herein designated VGAM PRECURSOR RNA, and of the ‘diced’ VGAM345 RNA, herein designated VGAM RNA, and a schematic representation of the secondary folding of VGAM345 folded precursor RNA, herein designated VGAM FOLDED PRECURSOR RNA, of VGAM345 are further described hereinbelow with reference to Table 1.

Nucleotide sequences of host target binding sites, such as BINDING SITE-I, BINDING SITE-II and BINDING SITE-III of FIG. 1, found on VGAM345 host target RNA, and schematic representation of the complementarity of each of these host target binding sites to VGAM345 RNA, herein designated VGAM RNA, are described hereinbelow with reference to Table 2.

As mentioned hereinabove with reference to FIG. 1, a function of VGAM345 gene, herein designated VGAM is inhibition of expression of VGAM345 target genes. It is appreciated that specific functions, and accordingly utilities, of VGAM345 correlate with, and may be deduced from, the identity of the target genes which VGAM345 binds and inhibits, and the function of these target genes, as elaborated hereinbelow.

Dickkopf Homolog 1 (Xenopus laevis) (DKK1, Accession NM_{—}012242) is a VGAM345 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM345 RNA, herein designated VGAM RNA, also designated SEQ ID:680.

A function of VGAM345 is therefore inhibition of Dickkopf Homolog 1 (Xenopus laevis) (DKK1, Accession NM_{—}012242), a gene which is a growth factor and inhibits Wnt signaling. Accordingly, utilities of VGAM345 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM345 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM345 RNA, herein designated VGAM RNA, also designated SEQ ID:680.

Another function of VGAM345 is therefore inhibition of Keratocan (KERA, Accession NM_{—}007035), a gene which may be important in developing and maintaining corneal transparency and for the structure of the stromal matrix. Accordingly, utilities of VGAM345 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM345 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM345 RNA, herein designated VGAM RNA, also designated SEQ ID:680.

Another function of VGAM345 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM345 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM345 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM345 RNA, herein designated VGAM RNA, also designated SEQ ID:680.

Another function of VGAM345 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM345 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM345 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM345 RNA, herein designated VGAM RNA, also designated SEQ ID:680.

Another function of VGAM345 is therefore inhibition of DKFZP56410422 (Accession NM_{—}031435). Accordingly, utilities of VGAM345 include diagnosis, prevention and treatment of diseases and clinical conditions associated with DKFZP56410422. Echinoderm Microtubule Associated Protein Like 4 (EML4, Accession NM_{—}019063) is another VGAM345 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM345 RNA, herein designated VGAM RNA, also designated SEQ ID:680.

Another function of VGAM345 is therefore inhibition of Echinoderm Microtubule Associated Protein Like 4 (EML4, Accession NM_{—}019063). Accordingly, utilities of VGAM345 include diagnosis, prevention and treatment of diseases and clinical conditions associated with EML4. FLJ21657 (Accession NM_{—}022483) is another VGAM345 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM345 RNA, herein designated VGAM RNA, also designated SEQ ID:680.

Another function of VGAM345 is therefore inhibition of FLJ21657 (Accession NM_{—}022483). Accordingly, utilities of VGAM345 include diagnosis, prevention and treatment of diseases and clinical conditions associated with FLJ21657. KIAA1054 (Accession XM_{—}043493) is another VGAM345 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM345 RNA, herein designated VGAM RNA, also designated SEQ ID:680.

Another function of VGAM345 is therefore inhibition of KIAA1054 (Accession XM_{—}043493). Accordingly, utilities of VGAM345 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM345 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1431, to the nucleotide sequence of VGAM345 RNA, herein designated VGAM RNA, also designated SEQ ID:680.

Another function of VGAM345 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM345 include diagnosis, prevention and treatment of diseases and clinical conditions associated with SEMA3E. LOC130507 (Accession XM_{—}059440) is another VGAM345 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM345 RNA, herein designated VGAM RNA, also designated SEQ ID:680.

Another function of VGAM345 is therefore inhibition of LOC130507 (Accession XM_{—}059440). Accordingly, utilities of VGAM345 include diagnosis, prevention and treatment of diseases and clinical conditions associated with LOC130507. LOC148145 (Accession XM_{—}097410) is another VGAM345 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM345 RNA, herein designated VGAM RNA, also designated SEQ ID:680.

Another function of VGAM345 is therefore inhibition of LOC148145 (Accession XM_{—}097410). Accordingly, utilities of VGAM345 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 346 (VGAM346) viral gene, which modulates expression of respective host target genes thereof, the function and utility of which host target genes is known in the art.

VGAM346 is a novel bioinformatically detected regulatory, non protein coding, viral micro RNA (miRNA) gene. The method by which VGAM346 was detected is described hereinabove with reference to FIGS. 1-8.

VGAM346 gene, herein designated VGAM GENE, is a viral gene contained in the genome of Vaccinia Virus. VGAM346 host target gene, herein designated VGAM HOST TARGET GENE, is a human gene contained in the human genome.

VGAM346 gene encodes a VGAM346 precursor RNA, herein designated VGAM PRECURSOR RNA. Similar to other miRNA genes, and unlike most ordinary genes, VGAM346 precursor RNA does not encode a protein. A nucleotide sequence identical or highly similar to the nucleotide sequence of VGAM346 precursor RNA is designated SEQ ID:332, and is provided hereinbelow with reference to the sequence listing part. Nucleotide sequence SEQ ID:332 is located at position 963 relative to the genome of Vaccinia Virus.

VGAM346 precursor RNA folds onto itself, forming VGAM346 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM346 folded precursor RNA into VGAM346 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM346 RNA is designated SEQ ID:681, and is provided hereinbelow with reference to the sequence listing part.

VGAM346 host target gene, herein designated VGAM HOST TARGET GENE, encodes a corresponding messenger RNA, VGAM346 host target RNA, herein designated VGAM HOST TARGET RNA. VGAM346 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM346 RNA, herein designated VGAM RNA, binds complementarily to one or more host target binding sites located in untranslated regions of VGAM346 host target RNA, herein designated VGAM HOST TARGET RNA. This complementary binding is due to the fact that the nucleotide sequence of VGAM346 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM346 RNA, herein designated VGAM RNA, may have a different number of host target binding sites in untranslated regions of a VGAM346 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM346 RNA, herein designated VGAM RNA, to host target binding sites on VGAM346 host target RNA, herein designated VGAM HOST TARGET RNA, such as BINDING SITE I, BINDING SITE II and BINDING SITE III, inhibits translation of VGAM346 host target RNA into VGAM346 host target protein, herein designated VGAM HOST TARGET PROTEIN. VGAM host target protein is therefore outlined by a broken line.

It is appreciated that VGAM346 host target gene, herein designated VGAM HOST TARGET GENE, in fact represents a plurality of VGAM346 host target genes. The mRNA of each one of this plurality of VGAM346 host target genes comprises one or more host target binding sites, each having a nucleotide sequence which is at least partly complementary to VGAM346 RNA, herein designated VGAM RNA, and which when bound by VGAM346 RNA causes inhibition of translation of respective one or more VGAM346 host target proteins.

It is further appreciated by one skilled in the art that the mode of translational inhibition illustrated by FIG. 1 with specific reference to translational inhibition exerted by VGAM346 gene, herein designated VGAM GENE, on one or more VGAM346 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM346 is inhibition of expression of host target genes, as part of a novel viral mechanism of attacking a host. Accordingly, utilities of VGAM346 include diagnosis, prevention and treatment of viral infection by Vaccinia Virus. Specific functions, and accordingly utilities, of VGAM346 correlate with, and may be deduced from, the identity of the host target genes which VGAM346 binds and inhibits, and the function of these host target genes, as elaborated hereinbelow.

Nucleotide sequences of the VGAM346 precursor RNA, herein designated VGAM PRECURSOR RNA, and of the ‘diced’ VGAM346 RNA, herein designated VGAM RNA, and a schematic representation of the secondary folding of VGAM346 folded precursor RNA, herein designated VGAM FOLDED PRECURSOR RNA, of VGAM346 are further described hereinbelow with reference to Table 1.

Nucleotide sequences of host target binding sites, such as BINDING SITE-I, BINDING SITE-II and BINDING SITE-III of FIG. 1, found on VGAM346 host target RNA, and schematic representation of the complementarity of each of these host target binding sites to VGAM346 RNA, herein designated VGAM RNA, are described hereinbelow with reference to Table 2.

As mentioned hereinabove with reference to FIG. 1, a function of VGAM346 gene, herein designated VGAM is inhibition of expression of VGAM346 target genes. It is appreciated that specific functions, and accordingly utilities, of VGAM346 correlate with, and may be deduced from, the identity of the target genes which VGAM346 binds and inhibits, and the function of these target genes, as elaborated hereinbelow.

Dickkopf Homolog 1 (Xenopus laevis) (DKK1, Accession NM_{—}012242) is a VGAM346 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM346 RNA, herein designated VGAM RNA, also designated SEQ ID:681.

A function of VGAM346 is therefore inhibition of Dickkopf Homolog 1 (Xenopus laevis) (DKK1, Accession NM_{—}012242), a gene which is a growth factor and inhibits Wnt signaling. Accordingly, utilities of VGAM346 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM346 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM346 RNA, herein designated VGAM RNA, also designated SEQ ID:681.

Another function of VGAM346 is therefore inhibition of Keratocan (KERA, Accession NM_{—}007035), a gene which may be important in developing and maintaining corneal transparency and for the structure of the stromal matrix. Accordingly, utilities of VGAM346 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM346 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM346 RNA, herein designated VGAM RNA, also designated SEQ ID:681.

Another function of VGAM346 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM346 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM346 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM346 RNA, herein designated VGAM RNA, also designated SEQ ID:681.

Another function of VGAM346 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM346 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM346 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM346 RNA, herein designated VGAM RNA, also designated SEQ ID:681.

Another function of VGAM346 is therefore inhibition of DKFZP56410422 (Accession NM_{—}031435). Accordingly, utilities of VGAM346 include diagnosis, prevention and treatment of diseases and clinical conditions associated with DKFZP56410422. Echinoderm Microtubule Associated Protein Like 4 (EML4, Accession NM_{—}019063) is another VGAM346 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM346 RNA, herein designated VGAM RNA, also designated SEQ ID:681.

Another function of VGAM346 is therefore inhibition of Echinoderm Microtubule Associated Protein Like 4 (EML4, Accession NM_{—}019063). Accordingly, utilities of VGAM346 include diagnosis, prevention and treatment of diseases and clinical conditions associated with EML4. FLJ21657 (Accession NM_{—}022483) is another VGAM346 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM346 RNA, herein designated VGAM RNA, also designated SEQ ID:681.

Another function of VGAM346 is therefore inhibition of FLJ21657 (Accession NM_{—}022483). Accordingly, utilities of VGAM346 include diagnosis, prevention and treatment of diseases and clinical conditions associated with FLJ21657. KIAA1054 (Accession XM_{—}043493) is another VGAM346 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM346 RNA, herein designated VGAM RNA, also designated SEQ ID:681.

Another function of VGAM346 is therefore inhibition of KIAA1054 (Accession XM_{—}043493). Accordingly, utilities of VGAM346 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM346 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM346 RNA, herein designated VGAM RNA, also designated SEQ ID:681.

Another function of VGAM346 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM346 include diagnosis, prevention and treatment of diseases and clinical conditions associated with SEMA3E. LOC130507 (Accession XM_{—}059440) is another VGAM346 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM346 RNA, herein designated VGAM RNA, also designated SEQ ID:681.

Another function of VGAM346 is therefore inhibition of LOC130507 (Accession XM_{—}059440). Accordingly, utilities of VGAM346 include diagnosis, prevention and treatment of diseases and clinical conditions associated with LOC130507. LOC148145 (Accession XM_{—}097410) is another VGAM346 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM346 RNA, herein designated VGAM RNA, also designated SEQ ID:681.

Another function of VGAM346 is therefore inhibition of LOC148145 (Accession XM_{—}097410). Accordingly, utilities of VGAM346 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 347 (VGAM347) viral gene, which modulates expression of respective host target genes thereof, the function and utility of which host target genes is known in the art.

VGAM347 is a novel bioinformatically detected regulatory, non protein coding, viral micro RNA (miRNA) gene. The method by which VGAM347 was detected is described hereinabove with reference to FIGS. 1-8.

VGAM347 gene, herein designated VGAM GENE, is a viral gene contained in the genome of Vaccinia Virus. VGAM347 host target gene, herein designated VGAM HOST TARGET GENE, is a human gene contained in the human genome.

VGAM347 gene encodes a VGAM347 precursor RNA, herein designated VGAM PRECURSOR RNA. Similar to other miRNA genes, and unlike most ordinary genes, VGAM347 precursor RNA does not encode a protein. A nucleotide sequence identical or highly similar to the nucleotide sequence of VGAM347 precursor RNA is designated SEQ ID:333, and is provided hereinbelow with reference to the sequence listing part. Nucleotide sequence SEQ ID:333 is located at position 963 relative to the genome of Vaccinia Virus.

VGAM347 precursor RNA folds onto itself, forming VGAM347 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM347 folded precursor RNA into VGAM347 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM347 RNA is designated SEQ ID:682, and is provided hereinbelow with reference to the sequence listing part.

VGAM347 host target gene, herein designated VGAM HOST TARGET GENE, encodes a corresponding messenger RNA, VGAM347 host target RNA, herein designated VGAM HOST TARGET RNA. VGAM347 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM347 RNA, herein designated VGAM RNA, binds complementarily to one or more host target binding sites located in untranslated regions of VGAM347 host target RNA, herein designated VGAM HOST TARGET RNA. This complementary binding is due to the fact that the nucleotide sequence of VGAM347 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM347 RNA, herein designated VGAM RNA, may have a different number of host target binding sites in untranslated regions of a VGAM347 host target RNA, herein designated VGAM HOST TARGET RNA. It is further appreciated that while FIG. 1 depicts host target binding sites in the 3′ UTR region, this is meant as an example only—these host target binding sites may be located in the 3′UTR region, the 5′UTR region, or in both 3′UTR and 5′UTR regions.

The complementary binding of VGAM347 RNA, herein designated VGAM RNA, to host target binding sites on VGAM347 host target RNA, herein designated VGAM HOST TARGET RNA, such as BINDING SITE I, BINDING SITE II and BINDING SITE III, inhibits translation of VGAM347 host target RNA into VGAM347 host target protein, herein designated VGAM HOST TARGET PROTEIN. VGAM host target protein is therefore outlined by a broken line.

It is appreciated that VGAM347 host target gene, herein designated VGAM HOST TARGET GENE, in fact represents a plurality of VGAM347 host target genes. The mRNA of each one of this plurality of VGAM347 host target genes comprises one or more host target binding sites, each having a nucleotide sequence which is at least partly complementary to VGAM347 RNA, herein designated VGAM RNA, and which when bound by VGAM347 RNA causes inhibition of translation of respective one or more VGAM347 host target proteins.

It is further appreciated by one skilled in the art that the mode of translational inhibition illustrated by FIG. 1 with specific reference to translational inhibition exerted by VGAM347 gene, herein designated VGAM GENE, on one or more VGAM347 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM347 is inhibition of expression of host target genes, as part of a novel viral mechanism of attacking a host. Accordingly, utilities of VGAM347 include diagnosis, prevention and treatment of viral infection by Vaccinia Virus. Specific functions, and accordingly utilities, of VGAM347 correlate with, and may be deduced from, the identity of the host target genes which VGAM347 binds and inhibits, and the function of these host target genes, as elaborated hereinbelow.

Nucleotide sequences of the VGAM347 precursor RNA, herein designated VGAM PRECURSOR RNA, and of the ‘diced’ VGAM347 RNA, herein designated VGAM RNA, and a schematic representation of the secondary folding of VGAM347 folded precursor RNA, herein designated VGAM FOLDED PRECURSOR RNA, of VGAM347 are further described hereinbelow with reference to Table 1.

Nucleotide sequences of host target binding sites, such as BINDING SITE-I, BINDING SITE-II and BINDING SITE-III of FIG. 1, found on VGAM347 host target RNA, and schematic representation of the complementarity of each of these host target binding sites to VGAM347 RNA, herein designated VGAM RNA, are described hereinbelow with reference to Table 2.

As mentioned hereinabove with reference to FIG. 1, a function of VGAM347 gene, herein designated VGAM is inhibition of expression of VGAM347 target genes. It is appreciated that specific functions, and accordingly utilities, of VGAM347 correlate with, and may be deduced from, the identity of the target genes which VGAM347 binds and inhibits, and the function of these target genes, as elaborated hereinbelow.

Dickkopf Homolog 1 (Xenopus laevis) (DKK1, Accession NM_{—}012242) is a VGAM347 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM347 RNA, herein designated VGAM RNA, also designated SEQ ID:682.

A function of VGAM347 is therefore inhibition of Dickkopf Homolog 1 (Xenopus laevis) (DKK1, Accession NM_{—}012242), a gene which is a growth factor and inhibits Wnt signaling. Accordingly, utilities of VGAM347 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM347 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM347 RNA, herein designated VGAM RNA, also designated SEQ ID:682.

Another function of VGAM347 is therefore inhibition of Keratocan (KERA, Accession NM_{—}007035), a gene which may be important in developing and maintaining corneal transparency and for the structure of the stromal matrix. Accordingly, utilities of VGAM347 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM347 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM347 RNA, herein designated VGAM RNA, also designated SEQ ID:682.

Another function of VGAM347 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM347 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM347 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM347 RNA, herein designated VGAM RNA, also designated SEQ ID:682.

Another function of VGAM347 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM347 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM347 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM347 RNA, herein designated VGAM RNA, also designated SEQ ID:682.

Another function of VGAM347 is therefore inhibition of DKFZP56410422 (Accession NM_{—}031435). Accordingly, utilities of VGAM347 include diagnosis, prevention and treatment of diseases and clinical conditions associated with DKFZP56410422. Echinoderm Microtubule Associated Protein Like 4 (EML4, Accession NM_{—}019063) is another VGAM347 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM347 RNA, herein designated VGAM RNA, also designated SEQ ID:682.

Another function of VGAM347 is therefore inhibition of Echinoderm Microtubule Associated Protein Like 4 (EML4, Accession NM_{—}019063). Accordingly, utilities of VGAM347 include diagnosis, prevention and treatment of diseases and clinical conditions associated with EML4. FLJ21657 (Accession NM_{—}022483) is another VGAM347 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM347 RNA, herein designated VGAM RNA, also designated SEQ ID:682.

Another function of VGAM347 is therefore inhibition of FLJ21657 (Accession NM_{—}022483). Accordingly, utilities of VGAM347 include diagnosis, prevention and treatment of diseases and clinical conditions associated with FLJ21657. KIAA1054 (Accession XM_{—}043493) is another VGAM347 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM347 RNA, herein designated VGAM RNA, also designated SEQ ID:682.

Another function of VGAM347 is therefore inhibition of KIAA1054 (Accession XM_{—}043493). Accordingly, utilities of VGAM347 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM347 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1431, to the nucleotide sequence of VGAM347 RNA, herein designated VGAM RNA, also designated SEQ ID:682.

Another function of VGAM347 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM347 include diagnosis, prevention and treatment of diseases and clinical conditions associated with SEMA3E. LOC130507 (Accession XM_{—}059440) is another VGAM347 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM347 RNA, herein designated VGAM RNA, also designated SEQ ID:682.

Another function of VGAM347 is therefore inhibition of LOC130507 (Accession XM_{—}059440). Accordingly, utilities of VGAM347 include diagnosis, prevention and treatment of diseases and clinical conditions associated with LOC130507. LOC148145 (Accession XM_{—}097410) is another VGAM347 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM347 RNA, herein designated VGAM RNA, also designated SEQ ID:682.

Another function of VGAM347 is therefore inhibition of LOC148145 (Accession XM_{—}097410). Accordingly, utilities of VGAM347 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 348 (VGAM348) viral gene, which modulates expression of respective host target genes thereof, the function and utility of which host target genes is known in the art.

VGAM348 is a novel bioinformatically detected regulatory, non protein coding, viral micro RNA (miRNA) gene. The method by which VGAM348 was detected is described hereinabove with reference to FIGS. 1-8.

VGAM348 gene, herein designated VGAM GENE, is a viral gene contained in the genome of Vaccinia Virus. VGAM348 host target gene, herein designated VGAM HOST TARGET GENE, is a human gene contained in the human genome.

VGAM348 gene encodes a VGAM348 precursor RNA, herein designated VGAM PRECURSOR RNA. Similar to other miRNA genes, and unlike most ordinary genes, VGAM348 precursor RNA does not encode a protein. A nucleotide sequence identical or highly similar to the nucleotide sequence of VGAM348 precursor RNA is designated SEQ ID:334, and is provided hereinbelow with reference to the sequence listing part. Nucleotide sequence SEQ ID:334 is located at position 963 relative to the genome of Vaccinia Virus.

VGAM348 precursor RNA folds onto itself, forming VGAM348 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM348 folded precursor RNA into VGAM348 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM348 RNA is designated SEQ ID:683, and is provided hereinbelow with reference to the sequence listing part.

VGAM348 host target gene, herein designated VGAM HOST TARGET GENE, encodes a corresponding messenger RNA, VGAM348 host target RNA, herein designated VGAM HOST TARGET RNA. VGAM348 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM348 RNA, herein designated VGAM RNA, binds complementarily to one or more host target binding sites located in untranslated regions of VGAM348 host target RNA, herein designated VGAM HOST TARGET RNA. This complementary binding is due to the fact that the nucleotide sequence of VGAM348 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM348 RNA, herein designated VGAM RNA, may have a different number of host target binding sites in untranslated regions of a VGAM348 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM348 RNA, herein designated VGAM RNA, to host target binding sites on VGAM348 host target RNA, herein designated VGAM HOST TARGET RNA, such as BINDING SITE I, BINDING SITE II and BINDING SITE III, inhibits translation of VGAM348 host target RNA into VGAM348 host target protein, herein designated VGAM HOST TARGET PROTEIN. VGAM host target protein is therefore outlined by a broken line.

It is appreciated that VGAM348 host target gene, herein designated VGAM HOST TARGET GENE, in fact represents a plurality of VGAM348 host target genes. The mRNA of each one of this plurality of VGAM348 host target genes comprises one or more host target binding sites, each having a nucleotide sequence which is at least partly complementary to VGAM348 RNA, herein designated VGAM RNA, and which when bound by VGAM348 RNA causes inhibition of translation of respective one or more VGAM348 host target proteins.

It is further appreciated by one skilled in the art that the mode of translational inhibition illustrated by FIG. 1 with specific reference to translational inhibition exerted by VGAM348 gene, herein designated VGAM GENE, on one or more VGAM348 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM348 is inhibition of expression of host target genes, as part of a novel viral mechanism of attacking a host. Accordingly, utilities of VGAM348 include diagnosis, prevention and treatment of viral infection by Vaccinia Virus. Specific functions, and accordingly utilities, of VGAM348 correlate with, and may be deduced from, the identity of the host target genes which VGAM348 binds and inhibits, and the function of these host target genes, as elaborated hereinbelow.

Nucleotide sequences of the VGAM348 precursor RNA, herein designated VGAM PRECURSOR RNA, and of the ‘diced’ VGAM348 RNA, herein designated VGAM RNA, and a schematic representation of the secondary folding of VGAM348 folded precursor RNA, herein designated VGAM FOLDED PRECURSOR RNA, of VGAM348 are further described hereinbelow with reference to Table 1.

Nucleotide sequences of host target binding sites, such as BINDING SITE-I, BINDING SITE-II and BINDING SITE-III of FIG. 1, found on VGAM348 host target RNA, and schematic representation of the complementarity of each of these host target binding sites to VGAM348 RNA, herein designated VGAM RNA, are described hereinbelow with reference to Table 2.

As mentioned hereinabove with reference to FIG. 1, a function of VGAM348 gene, herein designated VGAM is inhibition of expression of VGAM348 target genes. It is appreciated that specific functions, and accordingly utilities, of VGAM348 correlate with, and may be deduced from, the identity of the target genes which VGAM348 binds and inhibits, and the function of these target genes, as elaborated hereinbelow.

Dickkopf Homolog 1 (Xenopus laevis) (DKK1, Accession NM_{—}012242) is a VGAM348 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM348 RNA, herein designated VGAM RNA, also designated SEQ ID:683.

A function of VGAM348 is therefore inhibition of Dickkopf Homolog 1 (Xenopus laevis) (DKK1, Accession NM_{—}012242), a gene which is a growth factor and inhibits Wnt signaling. Accordingly, utilities of VGAM348 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM348 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM348 RNA, herein designated VGAM RNA, also designated SEQ ID:683.

Another function of VGAM348 is therefore inhibition of Keratocan (KERA, Accession NM_{—}007035), a gene which may be important in developing and maintaining corneal transparency and for the structure of the stromal matrix. Accordingly, utilities of VGAM348 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM348 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM348 RNA, herein designated VGAM RNA, also designated SEQ ID:683.

Another function of VGAM348 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM348 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM348 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM348 RNA, herein designated VGAM RNA, also designated SEQ ID:683.

Another function of VGAM348 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM348 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM348 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM348 RNA, herein designated VGAM RNA, also designated SEQ ID:683.

Another function of VGAM348 is therefore inhibition of DKFZP56410422 (Accession NM_{—}031435). Accordingly, utilities of VGAM348 include diagnosis, prevention and treatment of diseases and clinical conditions associated with DKFZP56410422. Echinoderm Microtubule Associated Protein Like 4 (EML4, Accession NM_{—}019063) is another VGAM348 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM348 RNA, herein designated VGAM RNA, also designated SEQ ID:683.

Another function of VGAM348 is therefore inhibition of Echinoderm Microtubule Associated Protein Like 4 (EML4, Accession NM_{—}019063). Accordingly, utilities of VGAM348 include diagnosis, prevention and treatment of diseases and clinical conditions associated with EML4. FLJ21657 (Accession NM_{—}022483) is another VGAM348 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM348 RNA, herein designated VGAM RNA, also designated SEQ ID:683.

Another function of VGAM348 is therefore inhibition of FLJ21657 (Accession NM_{—}022483). Accordingly, utilities of VGAM348 include diagnosis, prevention and treatment of diseases and clinical conditions associated with FLJ21657. KIAA1054 (Accession XM_{—}043493) is another VGAM348 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM348 RNA, herein designated VGAM RNA, also designated SEQ ID:683.

Another function of VGAM348 is therefore inhibition of KIAA1054 (Accession XM_{—}043493). Accordingly, utilities of VGAM348 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM348 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM348 RNA, herein designated VGAM RNA, also designated SEQ ID:683.

Another function of VGAM348 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM348 include diagnosis, prevention and treatment of diseases and clinical conditions associated with SEMA3E. LOC130507 (Accession XM_{—}059440) is another VGAM348 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM348 RNA, herein designated VGAM RNA, also designated SEQ ID:683.

Another function of VGAM348 is therefore inhibition of LOC130507 (Accession XM_{—}059440). Accordingly, utilities of VGAM348 include diagnosis, prevention and treatment of diseases and clinical conditions associated with LOC130507. LOC148145 (Accession XM_{—}097410) is another VGAM348 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM348 RNA, herein designated VGAM RNA, also designated SEQ ID:683.

Another function of VGAM348 is therefore inhibition of LOC148145 (Accession XM_{—}097410). Accordingly, utilities of VGAM348 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 349 (VGAM349) viral gene, which modulates expression of respective host target genes thereof, the function and utility of which host target genes is known in the art.

VGAM349 is a novel bioinformatically detected regulatory, non protein coding, viral micro RNA (miRNA) gene. The method by which VGAM349 was detected is described hereinabove with reference to FIGS. 1-8.

VGAM349 gene, herein designated VGAM GENE, is a viral gene contained in the genome of Vaccinia Virus. VGAM349 host target gene, herein designated VGAM HOST TARGET GENE, is a human gene contained in the human genome.

VGAM349 gene encodes a VGAM349 precursor RNA, herein designated VGAM PRECURSOR RNA. Similar to other miRNA genes, and unlike most ordinary genes, VGAM349 precursor RNA does not encode a protein. A nucleotide sequence identical or highly similar to the nucleotide sequence of VGAM349 precursor RNA is designated SEQ ID:335, and is provided hereinbelow with reference to the sequence listing part. Nucleotide sequence SEQ ID:335 is located at position 963 relative to the genome of Vaccinia Virus.

VGAM349 precursor RNA folds onto itself, forming VGAM349 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM349 folded precursor RNA into VGAM349 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM349 RNA is designated SEQ ID:684, and is provided hereinbelow with reference to the sequence listing part.

VGAM349 host target gene, herein designated VGAM HOST TARGET GENE, encodes a corresponding messenger RNA, VGAM349 host target RNA, herein designated VGAM HOST TARGET RNA. VGAM349 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM349 RNA, herein designated VGAM RNA, binds complementarily to one or more host target binding sites located in untranslated regions of VGAM349 host target RNA, herein designated VGAM HOST TARGET RNA. This complementary binding is due to the fact that the nucleotide sequence of VGAM349 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM349 RNA, herein designated VGAM RNA, may have a different number of host target binding sites in untranslated regions of a VGAM349 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM349 RNA, herein designated VGAM RNA, to host target binding sites on VGAM349 host target RNA, herein designated VGAM HOST TARGET RNA, such as BINDING SITE I, BINDING SITE II and BINDING SITE III, inhibits translation of VGAM349 host target RNA into VGAM349 host target protein, herein designated VGAM HOST TARGET PROTEIN. VGAM host target protein is therefore outlined by a broken line.

It is appreciated that VGAM349 host target gene, herein designated VGAM HOST TARGET GENE, in fact represents a plurality of VGAM349 host target genes. The mRNA of each one of this plurality of VGAM349 host target genes comprises one or more host target binding sites, each having a nucleotide sequence which is at least partly complementary to VGAM349 RNA, herein designated VGAM RNA, and which when bound by VGAM349 RNA causes inhibition of translation of respective one or more VGAM349 host target proteins.

It is further appreciated by one skilled in the art that the mode of translational inhibition illustrated by FIG. 1 with specific reference to translational inhibition exerted by VGAM349 gene, herein designated VGAM GENE, on one or more VGAM349 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM349 is inhibition of expression of host target genes, as part of a novel viral mechanism of attacking a host. Accordingly, utilities of VGAM349 include diagnosis, prevention and treatment of viral infection by Vaccinia Virus. Specific functions, and accordingly utilities, of VGAM349 correlate with, and may be deduced from, the identity of the host target genes which VGAM349 binds and inhibits, and the function of these host target genes, as elaborated hereinbelow.

Nucleotide sequences of the VGAM349 precursor RNA, herein designated VGAM PRECURSOR RNA, and of the ‘diced’ VGAM349 RNA, herein designated VGAM RNA, and a schematic representation of the secondary folding of VGAM349 folded precursor RNA, herein designated VGAM FOLDED PRECURSOR RNA, of VGAM349 are further described hereinbelow with reference to Table 1.

Nucleotide sequences of host target binding sites, such as BINDING SITE-I, BINDING SITE-II and BINDING SITE-III of FIG. 1, found on VGAM349 host target RNA, and schematic representation of the complementarity of each of these host target binding sites to VGAM349 RNA, herein designated VGAM RNA, are described hereinbelow with reference to Table 2.

As mentioned hereinabove with reference to FIG. 1, a function of VGAM349 gene, herein designated VGAM is inhibition of expression of VGAM349 target genes. It is appreciated that specific functions, and accordingly utilities, of VGAM349 correlate with, and may be deduced from, the identity of the target genes which VGAM349 binds and inhibits, and the function of these target genes, as elaborated hereinbelow.

Dickkopf Homolog 1 (Xenopus laevis) (DKK1, Accession NM_{—}012242) is a VGAM349 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM349 RNA, herein designated VGAM RNA, also designated SEQ ID:684.

A function of VGAM349 is therefore inhibition of Dickkopf Homolog 1 (Xenopus laevis) (DKK1, Accession NM_{—}012242), a gene which is a growth factor and inhibits Wnt signaling. Accordingly, utilities of VGAM349 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM349 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM349 RNA, herein designated VGAM RNA, also designated SEQ ID:684.

Another function of VGAM349 is therefore inhibition of Keratocan (KERA, Accession NM_{—}007035), a gene which may be important in developing and maintaining corneal transparency and for the structure of the stromal matrix. Accordingly, utilities of VGAM349 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM349 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM349 RNA, herein designated VGAM RNA, also designated SEQ ID:684.

Another function of VGAM349 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM349 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM349 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM349 RNA, herein designated VGAM RNA, also designated SEQ ID:684.

Another function of VGAM349 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM349 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM349 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM349 RNA, herein designated VGAM RNA, also designated SEQ ID:684.

Another function of VGAM349 is therefore inhibition of DKFZP56410422 (Accession NM_{—}031435). Accordingly, utilities of VGAM349 include diagnosis, prevention and treatment of diseases and clinical conditions associated with DKFZP56410422. Echinoderm Microtubule Associated Protein Like 4 (EML4, Accession NM_{—}019063) is another VGAM349 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM349 RNA, herein designated VGAM RNA, also designated SEQ ID:684.

Another function of VGAM349 is therefore inhibition of Echinoderm Microtubule Associated Protein Like 4 (EML4, Accession NM_{—}019063). Accordingly, utilities of VGAM349 include diagnosis, prevention and treatment of diseases and clinical conditions associated with EML4. FLJ21657 (Accession NM_{—}022483) is another VGAM349 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM349 RNA, herein designated VGAM RNA, also designated SEQ ID:684.

Another function of VGAM349 is therefore inhibition of FLJ21657 (Accession NM_{—}022483). Accordingly, utilities of VGAM349 include diagnosis, prevention and treatment of diseases and clinical conditions associated with FLJ21657. KIAA1054 (Accession XM_{—}043493) is another VGAM349 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM349 RNA, herein designated VGAM RNA, also designated SEQ ID:684.

Another function of VGAM349 is therefore inhibition of KIAA1054 (Accession XM_{—}043493). Accordingly, utilities of VGAM349 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM349 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM349 RNA, herein designated VGAM RNA, also designated SEQ ID:684.

Another function of VGAM349 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM349 include diagnosis, prevention and treatment of diseases and clinical conditions associated with SEMA3E. LOC130507 (Accession XM_{—}059440) is another VGAM349 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM349 RNA, herein designated VGAM RNA, also designated SEQ ID:684.

Another function of VGAM349 is therefore inhibition of LOC130507 (Accession XM_{—}059440). Accordingly, utilities of VGAM349 include diagnosis, prevention and treatment of diseases and clinical conditions associated with LOC130507. LOC148145 (Accession XM_{—}097410) is another VGAM349 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM349 RNA, herein designated VGAM RNA, also designated SEQ ID:684.

Another function of VGAM349 is therefore inhibition of LOC148145 (Accession XM_{—}097410). Accordingly, utilities of VGAM349 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 350 (VGAM350) viral gene, which modulates expression of respective host target genes thereof, the function and utility of which host target genes is known in the art.

VGAM350 is a novel bioinformatically detected regulatory, non protein coding, viral micro RNA (miRNA) gene. The method by which VGAM350 was detected is described hereinabove with reference to FIGS. 1-8.

VGAM350 gene, herein designated VGAM GENE, is a viral gene contained in the genome of Vaccinia Virus. VGAM350 host target gene, herein designated VGAM HOST TARGET GENE, is a human gene contained in the human genome.

VGAM350 gene encodes a VGAM350 precursor RNA, herein designated VGAM PRECURSOR RNA. Similar to other miRNA genes, and unlike most ordinary genes, VGAM350 precursor RNA does not encode a protein. A nucleotide sequence identical or highly similar to the nucleotide sequence of VGAM350 precursor RNA is designated SEQ ID:336, and is provided hereinbelow with reference to the sequence listing part. Nucleotide sequence SEQ ID:336 is located at position 963 relative to the genome of Vaccinia Virus.

VGAM350 precursor RNA folds onto itself, forming VGAM350 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM350 folded precursor RNA into VGAM350 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM350 RNA is designated SEQ ID:685, and is provided hereinbelow with reference to the sequence listing part.

VGAM350 host target gene, herein designated VGAM HOST TARGET GENE, encodes a corresponding messenger RNA, VGAM350 host target RNA, herein designated VGAM HOST TARGET RNA. VGAM350 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM350 RNA, herein designated VGAM RNA, binds complementarily to one or more host target binding sites located in untranslated regions of VGAM350 host target RNA, herein designated VGAM HOST TARGET RNA. This complementary binding is due to the fact that the nucleotide sequence of VGAM350 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM350 RNA, herein designated VGAM RNA, may have a different number of host target binding sites in untranslated regions of a VGAM350 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM350 RNA, herein designated VGAM RNA, to host target binding sites on VGAM350 host target RNA, herein designated VGAM HOST TARGET RNA, such as BINDING SITE I, BINDING SITE II and BINDING SITE III, inhibits translation of VGAM350 host target RNA into VGAM350 host target protein, herein designated VGAM HOST TARGET PROTEIN. VGAM host target protein is therefore outlined by a broken line.

It is appreciated that VGAM350 host target gene, herein designated VGAM HOST TARGET GENE, in fact represents a plurality of VGAM350 host target genes. The mRNA of each one of this plurality of VGAM350 host target genes comprises one or more host target binding sites, each having a nucleotide sequence which is at least partly complementary to VGAM350 RNA, herein designated VGAM RNA, and which when bound by VGAM350 RNA causes inhibition of translation of respective one or more VGAM350 host target proteins.

It is further appreciated by one skilled in the art that the mode of translational inhibition illustrated by FIG. 1 with specific reference to translational inhibition exerted by VGAM350 gene, herein designated VGAM GENE, on one or more VGAM350 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM350 is inhibition of expression of host target genes, as part of a novel viral mechanism of attacking a host. Accordingly, utilities of VGAM350 include diagnosis, prevention and treatment of viral infection by Vaccinia Virus. Specific functions, and accordingly utilities, of VGAM350 correlate with, and may be deduced from, the identity of the host target genes which VGAM350 binds and inhibits, and the function of these host target genes, as elaborated hereinbelow.

Nucleotide sequences of the VGAM350 precursor RNA, herein designated VGAM PRECURSOR RNA, and of the ‘diced’ VGAM350 RNA, herein designated VGAM RNA, and a schematic representation of the secondary folding of VGAM350 folded precursor RNA, herein designated VGAM FOLDED PRECURSOR RNA, of VGAM350 are further described hereinbelow with reference to Table 1.

Nucleotide sequences of host target binding sites, such as BINDING SITE-I, BINDING SITE-II and BINDING SITE-III of FIG. 1, found on VGAM350 host target RNA, and schematic representation of the complementarity of each of these host target binding sites to VGAM350 RNA, herein designated VGAM RNA, are described hereinbelow with reference to Table 2.

As mentioned hereinabove with reference to FIG. 1, a function of VGAM350 gene, herein designated VGAM is inhibition of expression of VGAM350 target genes. It is appreciated that specific functions, and accordingly utilities, of VGAM350 correlate with, and may be deduced from, the identity of the target genes which VGAM350 binds and inhibits, and the function of these target genes, as elaborated hereinbelow.

Dickkopf Homolog 1 (Xenopus laevis) (DKK1, Accession NM_{—}012242) is a VGAM350 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM350 RNA, herein designated VGAM RNA, also designated SEQ ID:685.

A function of VGAM350 is therefore inhibition of Dickkopf Homolog 1 (Xenopus laevis) (DKK1, Accession NM_{—}012242), a gene which is a growth factor and inhibits Wnt signaling. Accordingly, utilities of VGAM350 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM350 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM350 RNA, herein designated VGAM RNA, also designated SEQ ID:685.

Another function of VGAM350 is therefore inhibition of Keratocan (KERA, Accession NM_{—}007035), a gene which may be important in developing and maintaining corneal transparency and for the structure of the stromal matrix. Accordingly, utilities of VGAM350 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM350 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM350 RNA, herein designated VGAM RNA, also designated SEQ ID:685.

Another function of VGAM350 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM350 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM350 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM350 RNA, herein designated VGAM RNA, also designated SEQ ID:685.

Another function of VGAM350 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM350 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM350 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM350 RNA, herein designated VGAM RNA, also designated SEQ ID:685.

Another function of VGAM350 is therefore inhibition of DKFZP56410422 (Accession NM_{—}031435). Accordingly, utilities of VGAM350 include diagnosis, prevention and treatment of diseases and clinical conditions associated with DKFZP56410422. Echinoderm Microtubule Associated Protein Like 4 (EML4, Accession NM_{—}019063) is another VGAM350 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM350 RNA, herein designated VGAM RNA, also designated SEQ ID:685.

Another function of VGAM350 is therefore inhibition of Echinoderm Microtubule Associated Protein Like 4 (EML4, Accession NM_{—}019063). Accordingly, utilities of VGAM350 include diagnosis, prevention and treatment of diseases and clinical conditions associated with EML4. FLJ21657 (Accession NM_{—}022483) is another VGAM350 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM350 RNA, herein designated VGAM RNA, also designated SEQ ID:685.

Another function of VGAM350 is therefore inhibition of FLJ21657 (Accession NM_{—}022483). Accordingly, utilities of VGAM350 include diagnosis, prevention and treatment of diseases and clinical conditions associated with FLJ21657. KIAA1054 (Accession XM_{—}043493) is another VGAM350 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM350 RNA, herein designated VGAM RNA, also designated SEQ ID:685.

Another function of VGAM350 is therefore inhibition of KIAA1054 (Accession XM_{—}043493). Accordingly, utilities of VGAM350 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM350 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM350 RNA, herein designated VGAM RNA, also designated SEQ ID:685.

Another function of VGAM350 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM350 include diagnosis, prevention and treatment of diseases and clinical conditions associated with SEMA3E. LOC130507 (Accession XM_{—}059440) is another VGAM350 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM350 RNA, herein designated VGAM RNA, also designated SEQ ID:685.

Another function of VGAM350 is therefore inhibition of LOC130507 (Accession XM_{—}059440). Accordingly, utilities of VGAM350 include diagnosis, prevention and treatment of diseases and clinical conditions associated with LOC130507. LOC148145 (Accession XM_{—}097410) is another VGAM350 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM350 RNA, herein designated VGAM RNA, also designated SEQ ID:685.

Another function of VGAM350 is therefore inhibition of LOC148145 (Accession XM_{—}097410). Accordingly, utilities of VGAM350 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 351 (VGAM351) viral gene, which modulates expression of respective host target genes thereof, the function and utility of which host target genes is known in the art.

VGAM351 is a novel bioinformatically detected regulatory, non protein coding, viral micro RNA (miRNA) gene. The method by which VGAM351 was detected is described hereinabove with reference to FIGS. 1-8.

VGAM351 gene, herein designated VGAM GENE, is a viral gene contained in the genome of Vaccinia Virus. VGAM351 host target gene, herein designated VGAM HOST TARGET GENE, is a human gene contained in the human genome.

VGAM351 gene encodes a VGAM351 precursor RNA, herein designated VGAM PRECURSOR RNA. Similar to other miRNA genes, and unlike most ordinary genes, VGAM351 precursor RNA does not encode a protein. A nucleotide sequence identical or highly similar to the nucleotide sequence of VGAM351 precursor RNA is designated SEQ ID:337, and is provided hereinbelow with reference to the sequence listing part. Nucleotide sequence SEQ ID:337 is located at position 963 relative to the genome of Vaccinia Virus.

VGAM351 precursor RNA folds onto itself, forming VGAM351 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM351 folded precursor RNA into VGAM351 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM351 RNA is designated SEQ ID:686, and is provided hereinbelow with reference to the sequence listing part.

VGAM351 host target gene, herein designated VGAM HOST TARGET GENE, encodes a corresponding messenger RNA, VGAM351 host target RNA, herein designated VGAM HOST TARGET RNA. VGAM351 host target RNA comprises three regions, as is typical of mRNA of a protein coding gene: a 5′ untranslated region, a protein coding region and a 3′ untranslated region, designated 5′UTR, PROTEIN CODING and 3′ UTR respectively.

VGAM351 RNA, herein designated VGAM RNA, binds complementarily to one or more host target binding sites located in untranslated regions of VGAM351 host target RNA, herein designated VGAM HOST TARGET RNA. This complementary binding is due to the fact that the nucleotide sequence of VGAM351 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM351 RNA, herein designated VGAM RNA, may have a different number of host target binding sites in untranslated regions of a VGAM351 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM351 RNA, herein designated VGAM RNA, to host target binding sites on VGAM351 host target RNA, herein designated VGAM HOST TARGET RNA, such as BINDING SITE I, BINDING SITE II and BINDING SITE III, inhibits translation of VGAM351 host target RNA into VGAM351 host target protein, herein designated VGAM HOST TARGET PROTEIN. VGAM host target protein is therefore outlined by a broken line.

It is appreciated that VGAM351 host target gene, herein designated VGAM HOST TARGET GENE, in fact represents a plurality of VGAM351 host target genes. The mRNA of each one of this plurality of VGAM351 host target genes comprises one or more host target binding sites, each having a nucleotide sequence which is at least partly complementary to VGAM351 RNA, herein designated VGAM RNA, and which when bound by VGAM351 RNA causes inhibition of translation of respective one or more VGAM351 host target proteins.

It is further appreciated by one skilled in the art that the mode of translational inhibition illustrated by FIG. 1 with specific reference to translational inhibition exerted by VGAM351 gene, herein designated VGAM GENE, on one or more VGAM351 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM351 is inhibition of expression of host target genes, as part of a novel viral mechanism of attacking a host. Accordingly, utilities of VGAM351 include diagnosis, prevention and treatment of viral infection by Vaccinia Virus. Specific functions, and accordingly utilities, of VGAM351 correlate with, and may be deduced from, the identity of the host target genes which VGAM351 binds and inhibits, and the function of these host target genes, as elaborated hereinbelow.

Nucleotide sequences of the VGAM351 precursor RNA, herein designated VGAM PRECURSOR RNA, and of the ‘diced’ VGAM351 RNA, herein designated VGAM RNA, and a schematic representation of the secondary folding of VGAM351 folded precursor RNA, herein designated VGAM FOLDED PRECURSOR RNA, of VGAM351 are further described hereinbelow with reference to Table 1.

Nucleotide sequences of host target binding sites, such as BINDING SITE-I, BINDING SITE-II and BINDING SITE-III of FIG. 1, found on VGAM351 host target RNA, and schematic representation of the complementarity of each of these host target binding sites to VGAM351 RNA, herein designated VGAM RNA, are described hereinbelow with reference to Table 2.

As mentioned hereinabove with reference to FIG. 1, a function of VGAM351 gene, herein designated VGAM is inhibition of expression of VGAM351 target genes. It is appreciated that specific functions, and accordingly utilities, of VGAM351 correlate with, and may be deduced from, the identity of the target genes which VGAM351 binds and inhibits, and the function of these target genes, as elaborated hereinbelow.

Dickkopf Homolog 1 (Xenopus laevis) (DKK1, Accession NM_{—}012242) is a VGAM351 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM351 RNA, herein designated VGAM RNA, also designated SEQ ID:686.

A function of VGAM351 is therefore inhibition of Dickkopf Homolog 1 (Xenopus laevis) (DKK1, Accession NM_{—}012242), a gene which is a growth factor and inhibits Wnt signaling. Accordingly, utilities of VGAM351 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM351 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM351 RNA, herein designated VGAM RNA, also designated SEQ ID:686.

Another function of VGAM351 is therefore inhibition of Keratocan (KERA, Accession NM_{—}007035), a gene which may be important in developing and maintaining corneal transparency and for the structure of the stromal matrix. Accordingly, utilities of VGAM351 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM351 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM351 RNA, herein designated VGAM RNA, also designated SEQ ID:686.

Another function of VGAM351 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM351 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM351 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM351 RNA, herein designated VGAM RNA, also designated SEQ ID:686.

Another function of VGAM351 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM351 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM351 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM351 RNA, herein designated VGAM RNA, also designated SEQ ID:686.

Another function of VGAM351 is therefore inhibition of DKFZP56410422 (Accession NM_{—}031435). Accordingly, utilities of VGAM351 include diagnosis, prevention and treatment of diseases and clinical conditions associated with DKFZP56410422. Echinoderm Microtubule Associated Protein Like 4 (EML4, Accession NM_{—}019063) is another VGAM351 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM351 RNA, herein designated VGAM RNA, also designated SEQ ID:686.

Another function of VGAM351 is therefore inhibition of Echinoderm Microtubule Associated Protein Like 4 (EML4, Accession NM_{—}019063). Accordingly, utilities of VGAM351 include diagnosis, prevention and treatment of diseases and clinical conditions associated with EML4. FLJ21657 (Accession NM_{—}022483) is another VGAM351 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM351 RNA, herein designated VGAM RNA, also designated SEQ ID:686.

Another function of VGAM351 is therefore inhibition of FLJ21657 (Accession NM_{—}022483). Accordingly, utilities of VGAM351 include diagnosis, prevention and treatment of diseases and clinical conditions associated with FLJ21657. KIAA1054 (Accession XM_{—}043493) is another VGAM351 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM351 RNA, herein designated VGAM RNA, also designated SEQ ID:686.

Another function of VGAM351 is therefore inhibition of KIAA1054 (Accession XM_{—}043493). Accordingly, utilities of VGAM351 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM351 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID: 1431, to the nucleotide sequence of VGAM351 RNA, herein designated VGAM RNA, also designated SEQ ID:686.

Another function of VGAM351 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM351 include diagnosis, prevention and treatment of diseases and clinical conditions associated with SEMA3E. LOC130507 (Accession XM_{—}059440) is another VGAM351 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM351 RNA, herein designated VGAM RNA, also designated SEQ ID:686.

Another function of VGAM351 is therefore inhibition of LOC130507 (Accession XM_{—}059440). Accordingly, utilities of VGAM351 include diagnosis, prevention and treatment of diseases and clinical conditions associated with LOC130507. LOC148145 (Accession XM_{—}097410) is another VGAM351 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM351 RNA, herein designated VGAM RNA, also designated SEQ ID:686.

Another function of VGAM351 is therefore inhibition of LOC148145 (Accession XM_{—}097410). Accordingly, utilities of VGAM351 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 352 (VGAM352) viral gene, which modulates expression of respective host target genes thereof, the function and utility of which host target genes is known in the art.

VGAM352 is a novel bioinformatically detected regulatory, non protein coding, viral micro RNA (miRNA) gene. The method by which VGAM352 was detected is described hereinabove with reference to FIGS. 1-8.

VGAM352 gene, herein designated VGAM GENE, is a viral gene contained in the genome of Vaccinia Virus. VGAM352 host target gene, herein designated VGAM HOST TARGET GENE, is a human gene contained in the human genome.

VGAM352 gene encodes a VGAM352 precursor RNA, herein designated VGAM PRECURSOR RNA. Similar to other miRNA genes, and unlike most ordinary genes, VGAM352 precursor RNA does not encode a protein. A nucleotide sequence identical or highly similar to the nucleotide sequence of VGAM352 precursor RNA is designated SEQ ID:338, and is provided hereinbelow with reference to the sequence listing part. Nucleotide sequence SEQ ID:338 is located at position 2576 relative to the genome of Vaccinia Virus.

VGAM352 precursor RNA folds onto itself, forming VGAM352 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM352 folded precursor RNA into VGAM352 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM352 RNA is designated SEQ ID:687, and is provided hereinbelow with reference to the sequence listing part.

VGAM352 host target gene, herein designated VGAM HOST TARGET GENE, encodes a corresponding messenger RNA, VGAM352 host target RNA, herein designated VGAM HOST TARGET RNA. VGAM352 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM352 RNA, herein designated VGAM RNA, binds complementarily to one or more host target binding sites located in untranslated regions of VGAM352 host target RNA, herein designated VGAM HOST TARGET RNA. This complementary binding is due to the fact that the nucleotide sequence of VGAM352 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM352 RNA, herein designated VGAM RNA, may have a different number of host target binding sites in untranslated regions of a VGAM352 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM352 RNA, herein designated VGAM RNA, to host target binding sites on VGAM352 host target RNA, herein designated VGAM HOST TARGET RNA, such as BINDING SITE I, BINDING SITE II and BINDING SITE III, inhibits translation of VGAM352 host target RNA into VGAM352 host target protein, herein designated VGAM HOST TARGET PROTEIN. VGAM host target protein is therefore outlined by a broken line.

It is appreciated that VGAM352 host target gene, herein designated VGAM HOST TARGET GENE, in fact represents a plurality of VGAM352 host target genes. The mRNA of each one of this plurality of VGAM352 host target genes comprises one or more host target binding sites, each having a nucleotide sequence which is at least partly complementary to VGAM352 RNA, herein designated VGAM RNA, and which when bound by VGAM352 RNA causes inhibition of translation of respective one or more VGAM352 host target proteins.

It is further appreciated by one skilled in the art that the mode of translational inhibition illustrated by FIG. 1 with specific reference to translational inhibition exerted by VGAM352 gene, herein designated VGAM GENE, on one or more VGAM352 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM352 is inhibition of expression of host target genes, as part of a novel viral mechanism of attacking a host. Accordingly, utilities of VGAM352 include diagnosis, prevention and treatment of viral infection by Vaccinia Virus. Specific functions, and accordingly utilities, of VGAM352 correlate with, and may be deduced from, the identity of the host target genes which VGAM352 binds and inhibits, and the function of these host target genes, as elaborated hereinbelow.

Nucleotide sequences of the VGAM352 precursor RNA, herein designated VGAM PRECURSOR RNA, and of the ‘diced’ VGAM352 RNA, herein designated VGAM RNA, and a schematic representation of the secondary folding of VGAM352 folded precursor RNA, herein designated VGAM FOLDED PRECURSOR RNA, of VGAM352 are further described hereinbelow with reference to Table 1.

Nucleotide sequences of host target binding sites, such as BINDING SITE-I, BINDING SITE-II and BINDING SITE-III of FIG. 1, found on VGAM352 host target RNA, and schematic representation of the complementarity of each of these host target binding sites to VGAM352 RNA, herein designated VGAM RNA, are described hereinbelow with reference to Table 2.

As mentioned hereinabove with reference to FIG. 1, a function of VGAM352 gene, herein designated VGAM is inhibition of expression of VGAM352 target genes. It is appreciated that specific functions, and accordingly utilities, of VGAM352 correlate with, and may be deduced from, the identity of the target genes which VGAM352 binds and inhibits, and the function of these target genes, as elaborated hereinbelow.

Dickkopf Homolog 1 (Xenopus laevis) (DKK1, Accession NM_{—}012242) is a VGAM352 host target gene. DKK1 BINDING SITE is HOST TARGET binding site found in the 3′ untranslated region of mRNA encoded by DKK1, corresponding to a HOST TARGET binding site such as BINDING SITE I, BINDING SITE II or BINDING SITE III. Table 2 illustrates the complementarity of the nucleotide sequences of DKK1 BINDING SITE, designated SEQ ID: 1417, to the nucleotide sequence of VGAM352 RNA, herein designated VGAM RNA, also designated SEQ ID:687.

A function of VGAM352 is therefore inhibition of Dickkopf Homolog 1 (Xenopus laevis) (DKK1, Accession NM_{—}012242), a gene which is a growth factor and inhibits Wnt signaling. Accordingly, utilities of VGAM352 include diagnosis, prevention and treatment of diseases and clinical conditions associated with DKK1. The function of DKK1 and its association with various diseases and clinical conditions, has been established by previous studies, as described hereinabove with reference to VGAM39. Keratocan (KERA, Accession NM_{—}007035) is another VGAM352 host target gene. KERA BINDING SITE is HOST TARGET binding site found in the 3′ untranslated region of mRNA encoded by KERA, corresponding to a HOST TARGET binding site such as BINDING SITE I, BINDING SITE II or BINDING SITE III. Table 2 illustrates the complementarity of the nucleotide sequences of KERA BINDING SITE, designated SEQ ID:1349, to the nucleotide sequence of VGAM352 RNA, herein designated VGAM RNA, also designated SEQ ID:687.

Another function of VGAM352 is therefore inhibition of Keratocan (KERA, Accession NM_{—}007035), a gene which may be important in developing and maintaining corneal transparency and for the structure of the stromal matrix. Accordingly, utilities of VGAM352 include diagnosis, prevention and treatment of diseases and clinical conditions associated with KERA. The function of KERA and its association with various diseases and clinical conditions, has been established by previous studies, as described hereinabove with reference to VGAM39. Protein Phosphatase 1, Regulatory (inhibitor) Subunit 12A (PPP1R12A, Accession NM_{—}002480) is another VGAM352 host target gene. PPP1R12A BINDING SITE is HOST TARGET binding site found in the 3′ untranslated region of mRNA encoded by PPP1R12A, corresponding to a HOST TARGET binding site such as BINDING SITE I, BINDING SITE II or BINDING SITE III. Table 2 illustrates the complementarity of the nucleotide sequences of PPP1R12A BINDING SITE, designated SEQ ID:931, to the nucleotide sequence of VGAM352 RNA, herein designated VGAM RNA, also designated SEQ ID:687.

Another function of VGAM352 is therefore inhibition of Protein Phosphatase 1, Regulatory (inhibitor) Subunit 12A (PPP1R12A, Accession NM_{—}002480), a gene which regulates the interaction of actin and myosin. Accordingly, utilities of VGAM352 include diagnosis, prevention and treatment of diseases and clinical conditions associated with PPP1R12A. The function of PPP1R12A and its association with various diseases and clinical conditions, has been established by previous studies, as described hereinabove with reference to VGAM39. S100 Calcium Binding Protein, Beta (neural) (S100B, Accession NM_{—}006272) is another VGAM352 host target gene. S100B BINDING SITE is HOST TARGET binding site found in the 3′ untranslated region of mRNA encoded by S100B, corresponding to a HOST TARGET binding site such as BINDING SITE I, BINDING SITE II or BINDING SITE III. Table 2 illustrates the complementarity of the nucleotide sequences of S100B BINDING SITE, designated SEQ ID:1284, to the nucleotide sequence of VGAM352 RNA, herein designated VGAM RNA, also designated SEQ ID:687.

Another function of VGAM352 is therefore inhibition of S100 Calcium Binding Protein, Beta (neural) (S100B, Accession NM_{—}006272), a gene which weakly binds calcium but binds zinc very tightly-distinct binding sites with different affinities exist for both ions on each monomer. Accordingly, utilities of VGAM352 include diagnosis, prevention and treatment of diseases and clinical conditions associated with S100B. The function of S100B and its association with various diseases and clinical conditions, has been established by previous studies, as described hereinabove with reference to VGAM39. DKFZP56410422 (Accession NM_{—}031435) is another VGAM352 host target gene. DKFZP56410422 BINDING SITE is HOST TARGET binding site found in the 3′ untranslated region of mRNA encoded by DKFZP56410422, corresponding to a HOST TARGET binding site such as BINDING SITE I, BINDING SITE II or BINDING SITE III. Table 2 illustrates the complementarity of the nucleotide sequences of DKFZP56410422 BINDING SITE, designated SEQ ID:2196, to the nucleotide sequence of VGAM352 RNA, herein designated VGAM RNA, also designated SEQ ID:687.

Another function of VGAM352 is therefore inhibition of DKFZP56410422 (Accession NM_{—}031435). Accordingly, utilities of VGAM352 include diagnosis, prevention and treatment of diseases and clinical conditions associated with DKFZP56410422. Echinoderm Microtubule Associated Protein Like 4 (EML4, Accession NM_{—}019063) is another VGAM352 host target gene. EML4 BINDING SITE is HOST TARGET binding site found in the 3′ untranslated region of mRNA encoded by EML4, corresponding to a HOST TARGET binding site such as BINDING SITE I, BINDING SITE II or BINDING SITE III. Table 2 illustrates the complementarity of the nucleotide sequences of EML4 BINDING SITE, designated SEQ ID:1876, to the nucleotide sequence of VGAM352 RNA, herein designated VGAM RNA, also designated SEQ ID:687.

Another function of VGAM352 is therefore inhibition of Echinoderm Microtubule Associated Protein Like 4 (EML4, Accession NM_{—}019063). Accordingly, utilities of VGAM352 include diagnosis, prevention and treatment of diseases and clinical conditions associated with EML4. FLJ21657 (Accession NM_{—}022483) is another VGAM352 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID: 1990, to the nucleotide sequence of VGAM352 RNA, herein designated VGAM RNA, also designated SEQ ID:687.

Another function of VGAM352 is therefore inhibition of FLJ21657 (Accession NM_{—}022483). Accordingly, utilities of VGAM352 include diagnosis, prevention and treatment of diseases and clinical conditions associated with FLJ21657. KIAA1054 (Accession XM_{—}043493) is another VGAM352 host target gene. KIAA1054 BINDING SITE is HOST TARGET binding site found in the 3′ untranslated region of mRNA encoded by KIAA1054, corresponding to a HOST TARGET binding site such as BINDING SITE I, BINDING SITE II or BINDING SITE III. Table 2 illustrates the complementarity of the nucleotide sequences of KIAA1054 BINDING SITE, designated SEQ ID:2820, to the nucleotide sequence of VGAM352 RNA, herein designated VGAM RNA, also designated SEQ ID:687.

Another function of VGAM352 is therefore inhibition of KIAA1054 (Accession XM_{—}043493). Accordingly, utilities of VGAM352 include diagnosis, prevention and treatment of diseases and clinical conditions associated with KIAA1054. Sema Domain, Immunoglobulin Domain (Ig), Short Basic Domain, Secreted, (semaphorin) 3E (SEMA3E, Accession NM_{—}012431) is another VGAM352 host target gene. SEMA3E BINDING SITE is HOST TARGET binding site found in the 5′ untranslated region of mRNA encoded by SEMA3E, corresponding to a HOST TARGET binding site such as BINDING SITE I, BINDING SITE II or BINDING SITE III. Table 2 illustrates the complementarity of the nucleotide sequences of SEMA3E BINDING SITE, designated SEQ ID:1431, to the nucleotide sequence of VGAM352 RNA, herein designated VGAM RNA, also designated SEQ ID:687.

Another function of VGAM352 is therefore inhibition of Sema Domain, Immunoglobulin Domain (Ig), Short Basic Domain, Secreted, (semaphorin) 3E (SEMA3E, Accession NM_{—}012431). Accordingly, utilities of VGAM352 include diagnosis, prevention and treatment of diseases and clinical conditions associated with SEMA3E. LOC130507 (Accession XM_{—}059440) is another VGAM352 host target gene. LOC130507 BINDING SITE is HOST TARGET binding site found in the 3′ untranslated region of mRNA encoded by LOC130507, corresponding to a HOST TARGET binding site such as BINDING SITE I, BINDING SITE II or BINDING SITE III. Table 2 illustrates the complementarity of the nucleotide sequences of LOC130507 BINDING SITE, designated SEQ ID:3011, to the nucleotide sequence of VGAM352 RNA, herein designated VGAM RNA, also designated SEQ ID:687.

Another function of VGAM352 is therefore inhibition of LOC130507 (Accession XM_{—}059440). Accordingly, utilities of VGAM352 include diagnosis, prevention and treatment of diseases and clinical conditions associated with LOC130507. LOC148145 (Accession XM_{—}097410) is another VGAM352 host target gene. LOC148145 BINDING SITE is HOST TARGET binding site found in the 5′ untranslated region of mRNA encoded by LOC148145, corresponding to a HOST TARGET binding site such as BINDING SITE I, BINDING SITE II or BINDING SITE III. Table 2 illustrates the complementarity of the nucleotide sequences of LOC148145 BINDING SITE, designated SEQ ID:3296, to the nucleotide sequence of VGAM352 RNA, herein designated VGAM RNA, also designated SEQ ID:687.

Another function of VGAM352 is therefore inhibition of LOC148145 (Accession XM_{—}097410). Accordingly, utilities of VGAM352 include diagnosis, prevention and treatment of diseases and clinical conditions associated with LOC148145. FIG. 1 further provides a conceptual description of a novel bioinformatically detected viral gene of the present invention, referred to here as Viral Genomic Address Messenger 353 (VGAM353) viral gene, which modulates expression of respective host target genes thereof, the function and utility of which host target genes is known in the art.

VGAM353 is a novel bioinformatically detected regulatory, non protein coding, viral micro RNA (miRNA) gene. The method by which VGAM353 was detected is described hereinabove with reference to FIGS. 1-8.

VGAM353 gene, herein designated VGAM GENE, is a viral gene contained in the genome of Vaccinia Virus. VGAM353 host target gene, herein designated VGAM HOST TARGET GENE, is a human gene contained in the human genome.

VGAM353 gene encodes a VGAM353 precursor RNA, herein designated VGAM PRECURSOR RNA. Similar to other miRNA genes, and unlike most ordinary genes, VGAM353 precursor RNA does not encode a protein. A nucleotide sequence identical or highly similar to the nucleotide sequence of VGAM353 precursor RNA is designated SEQ ID:339, and is provided hereinbelow with reference to the sequence listing part. Nucleotide sequence SEQ ID:339 is located at position 4213 relative to the genome of Vaccinia Virus.

VGAM353 precursor RNA folds onto itself, forming VGAM353 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM353 folded precursor RNA into VGAM353 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM353 RNA is designated SEQ ID:688, and is provided hereinbelow with reference to the sequence listing part.

VGAM353 host target gene, herein designated VGAM HOST TARGET GENE, encodes a corresponding messenger RNA, VGAM353 host target RNA, herein designated VGAM HOST TARGET RNA. VGAM353 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM353 RNA, herein designated VGAM RNA, binds complementarily to one or more host target binding sites located in untranslated regions of VGAM353 host target RNA, herein designated VGAM HOST TARGET RNA. This complementary binding is due to the fact that the nucleotide sequence of VGAM353 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM353 RNA, herein designated VGAM RNA, may have a different number of host target binding sites in untranslated regions of a VGAM353 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM353 RNA, herein designated VGAM RNA, to host target binding sites on VGAM353 host target RNA, herein designated VGAM HOST TARGET RNA, such as BINDING SITE I, BINDING SITE II and BINDING SITE III, inhibits translation of VGAM353 host target RNA into VGAM353 host target protein, herein designated VGAM HOST TARGET PROTEIN. VGAM host target protein is therefore outlined by a broken line.

It is appreciated that VGAM353 host target gene, herein designated VGAM HOST TARGET GENE, in fact represents a plurality of VGAM353 host target genes. The mRNA of each one of this plurality of VGAM353 host target genes comprises one or more host target binding sites, each having a nucleotide sequence which is at least partly complementary to VGAM353 RNA, herein designated VGAM RNA, and which when bound by VGAM353 RNA causes inhibition of translation of respective one or more VGAM353 host target proteins.

It is further appreciated by one skilled in the art that the mode of translational inhibition illustrated by FIG. 1 with specific reference to translational inhibition exerted by VGAM353 gene, herein designated VGAM GENE, on one or more VGAM353 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM353 is inhibition of expression of host target genes, as part of a novel viral mechanism of attacking a host. Accordingly, utilities of VGAM353 include diagnosis, prevention and treatment of viral infection by Vaccinia Virus. Specific functions, and accordingly utilities, of VGAM353 correlate with, and may be deduced from, the identity of the host target genes which VGAM353 binds and inhibits, and the function of these host target genes, as elaborated hereinbelow.

Nucleotide sequences of the VGAM353 precursor RNA, herein designated VGAM PRECURSOR RNA, and of the ‘diced’ VGAM353 RNA, herein designated VGAM RNA, and a schematic representation of the secondary folding of VGAM353 folded precursor RNA, herein designated VGAM FOLDED PRECURSOR RNA, of VGAM353 are further described hereinbelow with reference to Table 1.

Nucleotide sequences of host target binding sites, such as BINDING SITE-I, BINDING SITE-II and BINDING SITE-III of FIG. 1, found on VGAM353 host target RNA, and schematic representation of the complementarity of each of these host target binding sites to VGAM353 RNA, herein designated VGAM RNA, are described hereinbelow with reference to Table 2.

As mentioned hereinabove with reference to FIG. 1, a function of VGAM353 gene, herein designated VGAM is inhibition of expression of VGAM353 target genes. It is appreciated that specific functions, and accordingly utilities, of VGAM353 correlate with, and may be deduced from, the identity of the target genes which VGAM353 binds and inhibits, and the function of these target genes, as elaborated hereinbelow.

EFG1 (Accession XM_{—}170611) is a VGAM353 host target gene. EFG1 BINDING SITE is HOST TARGET binding site found in the 3′ untranslated region of mRNA encoded by EFG1, corresponding to a HOST TARGET binding site such as BINDING SITE I, BINDING SITE II or BINDING SITE III. Table 2 illustrates the complementarity of the nucleotide sequences of EFG1 BINDING SITE, designated SEQ ID:3661, to the nucleotide sequence of VGAM353 RNA, herein designated VGAM RNA, also designated SEQ ID:688.

A function of VGAM353 is therefore inhibition of EFG1 (Accession XM_{—}170611), a gene which promotes the gtp-dependent translocation of the nascent protein chain from the a-site to the p-site of the ribosome in the mitochondria. Accordingly, utilities of VGAM353 include diagnosis, prevention and treatment of diseases and clinical conditions associated with EFG1. The function of EFG1 and its association with various diseases and clinical conditions, has been established by previous studies, as described hereinabove with reference to VGAM46. Phytanoyl-CoA Hydroxylase (Refsum disease) (PHYH, Accession NM_{—}006214) is another VGAM353 host target gene. PHYH BINDING SITE is HOST TARGET binding site found in the 3′ untranslated region of mRNA encoded by PHYH, corresponding to a HOST TARGET binding site such as BINDING SITE I, BINDING SITE II or BINDING SITE III. Table 2 illustrates the complementarity of the nucleotide sequences of PHYH BINDING SITE, designated SEQ ID:1281, to the nucleotide sequence of VGAM353 RNA, herein designated VGAM RNA, also designated SEQ ID:688.

Another function of VGAM353 is therefore inhibition of Phytanoyl-CoA Hydroxylase (Refsum disease) (PHYH, Accession NM_{—}006214). Accordingly, utilities of VGAM353 include diagnosis, prevention and treatment of diseases and clinical conditions associated with PHYH. Solute Carrier Family 6 (neurotransmitter transporter, dopamine), Member 3 (SLC6A3, Accession NM_{—}001044) is another VGAM353 host target gene. SLC6A3 BINDING SITE is HOST TARGET binding site found in the 3′ untranslated region of mRNA encoded by SLC6A3, corresponding to a HOST TARGET binding site such as BINDING SITE I, BINDING SITE II or BINDING SITE III. Table 2 illustrates the complementarity of the nucleotide sequences of SLC6A3 BINDING SITE, designated SEQ ID:795, to the nucleotide sequence of VGAM353 RNA, herein designated VGAM RNA, also designated SEQ ID:688.

Another function of VGAM353 is therefore inhibition of Solute Carrier Family 6 (neurotransmitter transporter, dopamine), Member 3 (SLC6A3, Accession NM_{—}001044), a gene which terminates the action of dopamine by its high affinity sodium-dependent reuptake into presynaptic terminals. Accordingly, utilities of VGAM353 include diagnosis, prevention and treatment of diseases and clinical conditions associated with SLC6A3. The function of SLC6A3 and its association with various diseases and clinical conditions, has been established by previous studies, as described hereinabove with reference to VGAM46. H2AV (Accession NM_{—}138635) is another VGAM353 host target gene. H2AV BINDING SITE is HOST TARGET binding site found in the 3′ untranslated region of mRNA encoded by H2AV, corresponding to a HOST TARGET binding site such as BINDING SITE I, BINDING SITE II or BINDING SITE III. Table 2 illustrates the complementarity of the nucleotide sequences of H2AV BINDING SITE, designated SEQ ID:2447, to the nucleotide sequence of VGAM353 RNA, herein designated VGAM RNA, also designated SEQ ID:688.

Another function of VGAM353 is therefore inhibition of H2AV (Accession NM_{—}138635). Accordingly, utilities of VGAM353 include diagnosis, prevention and treatment of diseases and clinical conditions associated with H2AV. HMP19 (Accession XM_{—}113455) is another VGAM353 host target gene. HMP19 BINDING SITE is HOST TARGET binding site found in the 3′ untranslated region of mRNA encoded by HMP19, corresponding to a HOST TARGET binding site such as BINDING SITE I, BINDING SITE II or BINDING SITE III. Table 2 illustrates the complementarity of the nucleotide sequences of HMP19 BINDING SITE, designated SEQ ID:3408, to the nucleotide sequence of VGAM353 RNA, herein designated VGAM RNA, also designated SEQ ID:688.

Another function of VGAM353 is therefore inhibition of HMP19 (Accession XM_{—}113455). Accordingly, utilities of VGAM353 include diagnosis, prevention and treatment of diseases and clinical conditions associated with HMP19. KIAA1726 (Accession XM_{—}040860) is another VGAM353 host target gene. KIAA1726 BINDING SITE is HOST TARGET binding site found in the 3′ untranslated region of mRNA encoded by KIAA1726, corresponding to a HOST TARGET binding site such as BINDING SITE I, BINDING SITE II or BINDING SITE III. Table 2 illustrates the complementarity of the nucleotide sequences of KIAA1726 BINDING SITE, designated SEQ ID:2786, to the nucleotide sequence of VGAM353 RNA, herein designated VGAM RNA, also designated SEQ ID:688.

Another function of VGAM353 is therefore inhibition of KIAA1726 (Accession XM_{—}040860). Accordingly, utilities of VGAM353 include diagnosis, prevention and treatment of diseases and clinical conditions associated with KIAA1726. FIG. 1 further provides a conceptual description of a novel bioinformatically detected viral gene of the present invention, referred to here as Viral Genomic Address Messenger 354 (VGAM354) viral gene, which modulates expression of respective host target genes thereof, the function and utility of which host target genes is known in the art.

VGAM354 is a novel bioinformatically detected regulatory, non protein coding, viral micro RNA (miRNA) gene. The method by which VGAM354 was detected is described hereinabove with reference to FIGS. 1-8.

VGAM354 gene, herein designated VGAM GENE, is a viral gene contained in the genome of Vaccinia Virus. VGAM354 host target gene, herein designated VGAM HOST TARGET GENE, is a human gene contained in the human genome.

VGAM354 gene encodes a VGAM354 precursor RNA, herein designated VGAM PRECURSOR RNA. Similar to other miRNA genes, and unlike most ordinary genes, VGAM354 precursor RNA does not encode a protein. A nucleotide sequence identical or highly similar to the nucleotide sequence of VGAM354 precursor RNA is designated SEQ ID:340, and is provided hereinbelow with reference to the sequence listing part. Nucleotide sequence SEQ ID:340 is located at position 3249 relative to the genome of Vaccinia Virus.

VGAM354 precursor RNA folds onto itself, forming VGAM354 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM354 folded precursor RNA into VGAM354 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 76%) nucleotide sequence of VGAM354 RNA is designated SEQ ID:689, and is provided hereinbelow with reference to the sequence listing part.

VGAM354 host target gene, herein designated VGAM HOST TARGET GENE, encodes a corresponding messenger RNA, VGAM354 host target RNA, herein designated VGAM HOST TARGET RNA. VGAM354 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM354 RNA, herein designated VGAM RNA, binds complementarily to one or more host target binding sites located in untranslated regions of VGAM354 host target RNA, herein designated VGAM HOST TARGET RNA. This complementary binding is due to the fact that the nucleotide sequence of VGAM354 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM354 RNA, herein designated VGAM RNA, may have a different number of host target binding sites in untranslated regions of a VGAM354 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM354 RNA, herein designated VGAM RNA, to host target binding sites on VGAM354 host target RNA, herein designated VGAM HOST TARGET RNA, such as BINDING SITE I, BINDING SITE II and BINDING SITE III, inhibits translation of VGAM354 host target RNA into VGAM354 host target protein, herein designated VGAM HOST TARGET PROTEIN. VGAM host target protein is therefore outlined by a broken line.

It is appreciated that VGAM354 host target gene, herein designated VGAM HOST TARGET GENE, in fact represents a plurality of VGAM354 host target genes. The mRNA of each one of this plurality of VGAM354 host target genes comprises one or more host target binding sites, each having a nucleotide sequence which is at least partly complementary to VGAM354 RNA, herein designated VGAM RNA, and which when bound by VGAM354 RNA causes inhibition of translation of respective one or more VGAM354 host target proteins.

It is further appreciated by one skilled in the art that the mode of translational inhibition illustrated by FIG. 1 with specific reference to translational inhibition exerted by VGAM354 gene, herein designated VGAM GENE, on one or more VGAM354 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM354 is inhibition of expression of host target genes, as part of a novel viral mechanism of attacking a host. Accordingly, utilities of VGAM354 include diagnosis, prevention and treatment of viral infection by Vaccinia Virus. Specific functions, and accordingly utilities, of VGAM354 correlate with, and may be deduced from, the identity of the host target genes which VGAM354 binds and inhibits, and the function of these host target genes, as elaborated hereinbelow.

Nucleotide sequences of the VGAM354 precursor RNA, herein designated VGAM PRECURSOR RNA, and of the ‘diced’ VGAM354 RNA, herein designated VGAM RNA, and a schematic representation of the secondary folding of VGAM354 folded precursor RNA, herein designated VGAM FOLDED PRECURSOR RNA, of VGAM354 are further described hereinbelow with reference to Table 1.

Nucleotide sequences of host target binding sites, such as BINDING SITE-I, BINDING SITE-II and BINDING SITE-III of FIG. 1, found on VGAM354 host target RNA, and schematic representation of the complementarity of each of these host target binding sites to VGAM354 RNA, herein designated VGAM RNA, are described hereinbelow with reference to Table 2.

As mentioned hereinabove with reference to FIG. 1, a function of VGAM354 gene, herein designated VGAM is inhibition of expression of VGAM354 target genes. It is appreciated that specific functions, and accordingly utilities, of VGAM354 correlate with, and may be deduced from, the identity of the target genes which VGAM354 binds and inhibits, and the function of these target genes, as elaborated hereinbelow.

SRY (sex determining region Y)-box 11 (SOX11, Accession NM_{—}003108) is a VGAM354 host target gene. SOX11 BINDING SITE is HOST TARGET binding site found in the 3′ untranslated region of mRNA encoded by SOX11, corresponding to a HOST TARGET binding site such as BINDING SITE I, BINDING SITE II or BINDING SITE III. Table 2 illustrates the complementarity of the nucleotide sequences of SOX11 BINDING SITE, designated SEQ ID:992, to the nucleotide sequence of VGAM354 RNA, herein designated VGAM RNA, also designated SEQ ID:689.

A function of VGAM354 is therefore inhibition of SRY (sex determining region Y)-box 11 (SOX11, Accession NM_{—}003108). Accordingly, utilities of VGAM354 include diagnosis, prevention and treatment of diseases and clinical conditions associated with SOX11. CAP350 (Accession NM_{—}014810) is another VGAM354 host target gene. CAP350 BINDING SITE is HOST TARGET binding site found in the 3′ untranslated region of mRNA encoded by CAP350, corresponding to a HOST TARGET binding site such as BINDING SITE I, BINDING SITE II or BINDING SITE III. Table 2 illustrates the complementarity of the nucleotide sequences of CAP350 BINDING SITE, designated SEQ ID:1554, to the nucleotide sequence of VGAM354 RNA, herein designated VGAM RNA, also designated SEQ ID:689.

Another function of VGAM354 is therefore inhibition of CAP350 (Accession NM_{—}014810). Accordingly, utilities of VGAM354 include diagnosis, prevention and treatment of diseases and clinical conditions associated with CAP350. DKFZP564A022 (Accession NM_{—}030954) is another VGAM354 host target gene. DKFZP564A022 BINDING SITE is HOST TARGET binding site found in the 3′ untranslated region of mRNA encoded by DKFZP564A022, corresponding to a HOST TARGET binding site such as BINDING SITE I, BINDING SITE II or BINDING SITE III. Table 2 illustrates the complementarity of the nucleotide sequences of DKFZP564A022 BINDING SITE, designated SEQ ID:2174, to the nucleotide sequence of VGAM354 RNA, herein designated VGAM RNA, also designated SEQ ID:689.

Another function of VGAM354 is therefore inhibition of DKFZP564A022 (Accession NM_{—}030954). Accordingly, utilities of VGAM354 include diagnosis, prevention and treatment of diseases and clinical conditions associated with DKFZP564A022. FLJ11730 (Accession NM_{—}022756) is another VGAM354 host target gene. FLJ11730 BINDING SITE is HOST TARGET binding site found in the 3′ untranslated region of mRNA encoded by FLJ11730, corresponding to a HOST TARGET binding site such as BINDING SITE I, BINDING SITE II or BINDING SITE III. Table 2 illustrates the complementarity of the nucleotide sequences of FLJ11730 BINDING SITE, designated SEQ ID:2005, to the nucleotide sequence of VGAM354 RNA, herein designated VGAM RNA, also designated SEQ ID:689.

Another function of VGAM354 is therefore inhibition of FLJ11730 (Accession NM_{—}022756). Accordingly, utilities of VGAM354 include diagnosis, prevention and treatment of diseases and clinical conditions associated with FLJ11730. FLJ20686 (Accession NM_{—}017925) is another VGAM354 host target gene. FLJ20686 BINDING SITE is HOST TARGET binding site found in the 5′ untranslated region of mRNA encoded by FLJ20686, corresponding to a HOST TARGET binding site such as BINDING SITE I, BINDING SITE II or BINDING SITE III. Table 2 illustrates the complementarity of the nucleotide sequences of FLJ20686 BINDING SITE, designated SEQ ID:1765, to the nucleotide sequence of VGAM354 RNA, herein designated VGAM RNA, also designated SEQ ID:689.

Another function of VGAM354 is therefore inhibition of FLJ20686 (Accession NM_{—}017925). Accordingly, utilities of VGAM354 include diagnosis, prevention and treatment of diseases and clinical conditions associated with FLJ20686. FRSB (Accession NM_{—}005687) is another VGAM354 host target gene. FRSB BINDING SITE is HOST TARGET binding site found in the 3′ untranslated region of mRNA encoded by FRSB, corresponding to a HOST TARGET binding site such as BINDING SITE I, BINDING SITE II or BINDING SITE III. Table 2 illustrates the complementarity of the nucleotide sequences of FRSB BINDING SITE, designated SEQ ID:1236, to the nucleotide sequence of VGAM354 RNA, herein designated VGAM RNA, also designated SEQ ID:689.

Another function of VGAM354 is therefore inhibition of FRSB (Accession NM_{—}005687). Accordingly, utilities of VGAM354 include diagnosis, prevention and treatment of diseases and clinical conditions associated with FRSB. KIAA1600 (Accession XM_{—}049351) is another VGAM354 host target gene. KIAA1600 BINDING SITE is HOST TARGET binding site found in the 3′ untranslated region of mRNA encoded by KIAA1600, corresponding to a HOST TARGET binding site such as BINDING SITE I, BINDING SITE II or BINDING SITE III. Table 2 illustrates the complementarity of the nucleotide sequences of KIAA1600 BINDING SITE, designated SEQ ID:2919, to the nucleotide sequence of VGAM354 RNA, herein designated VGAM RNA, also designated SEQ ID:689.

Another function of VGAM354 is therefore inhibition of KIAA1600 (Accession XM_{—}049351). Accordingly, utilities of VGAM354 include diagnosis, prevention and treatment of diseases and clinical conditions associated with KIAA1600. LOC124801 (Accession XM_{—}058850) is another VGAM354 host target gene. LOC124801 BINDING SITE is HOST TARGET binding site found in the 3′ untranslated region of mRNA encoded by LOC124801, corresponding to a HOST TARGET binding site such as BINDING SITE I, BINDING SITE II or BINDING SITE III. Table 2 illustrates the complementarity of the nucleotide sequences of LOC124801 BINDING SITE, designated SEQ ID:2996, to the nucleotide sequence of VGAM354 RNA, herein designated VGAM RNA, also designated SEQ ID:689.

Another function of VGAM354 is therefore inhibition of LOC124801 (Accession XM_{—}058850). Accordingly, utilities of VGAM354 include diagnosis, prevention and treatment of diseases and clinical conditions associated with LOC124801. LOC221035 (Accession XM_{—}167640) is another VGAM354 host target gene. LOC221035 BINDING SITE is HOST TARGET binding site found in the 3′ untranslated region of mRNA encoded by LOC221035, corresponding to a HOST TARGET binding site such as BINDING SITE I, BINDING SITE II or BINDING SITE III. Table 2 illustrates the complementarity of the nucleotide sequences of LOC221035 BINDING SITE, designated SEQ ID:3593, to the nucleotide sequence of VGAM354 RNA, herein designated VGAM RNA, also designated SEQ ID:689.

Another function of VGAM354 is therefore inhibition of LOC221035 (Accession XM_{—}167640). Accordingly, utilities of VGAM354 include diagnosis, prevention and treatment of diseases and clinical conditions associated with LOC221035. LOC51611 (Accession NM_{—}015958) is another VGAM354 host target gene. LOC51611 BINDING SITE is HOST TARGET binding site found in the 3′ untranslated region of mRNA encoded by LOC51611, corresponding to a HOST TARGET binding site such as BINDING SITE I, BINDING SITE II or BINDING SITE III. Table 2 illustrates the complementarity of the nucleotide sequences of LOC51611 BINDING SITE, designated SEQ ID:1653, to the nucleotide sequence of VGAM354 RNA, herein designated VGAM RNA, also designated SEQ ID:689.

Another function of VGAM354 is therefore inhibition of LOC51611 (Accession NM_{—}015958). Accordingly, utilities of VGAM354 include diagnosis, prevention and treatment of diseases and clinical conditions associated with LOC51611. FIG. 1 further provides a conceptual description of a novel bioinformatically detected viral gene of the present invention, referred to here as Viral Genomic Address Messenger 355 (VGAM355) viral gene, which modulates expression of respective host target genes thereof, the function and utility of which host target genes is known in the art.

VGAM355 is a novel bioinformatically detected regulatory, non protein coding, viral micro RNA (miRNA) gene. The method by which VGAM355 was detected is described hereinabove with reference to FIGS. 1-8.

VGAM355 gene, herein designated VGAM GENE, is a viral gene contained in the genome of Vaccinia Virus. VGAM355 host target gene, herein designated VGAM HOST TARGET GENE, is a human gene contained in the human genome.

VGAM355 gene encodes a VGAM355 precursor RNA, herein designated VGAM PRECURSOR RNA. Similar to other miRNA genes, and unlike most ordinary genes, VGAM355 precursor RNA does not encode a protein. A nucleotide sequence identical or highly similar to the nucleotide sequence of VGAM355 precursor RNA is designated SEQ ID:341, and is provided hereinbelow with reference to the sequence listing part. Nucleotide sequence SEQ ID:341 is located at position 3686 relative to the genome of Vaccinia Virus.

VGAM355 precursor RNA folds onto itself, forming VGAM355 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM355 folded precursor RNA into VGAM355 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 74%) nucleotide sequence of VGAM355 RNA is designated SEQ ID:690, and is provided hereinbelow with reference to the sequence listing part.

VGAM355 host target gene, herein designated VGAM HOST TARGET GENE, encodes a corresponding messenger RNA, VGAM355 host target RNA, herein designated VGAM HOST TARGET RNA. VGAM355 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM355 RNA, herein designated VGAM RNA, binds complementarily to one or more host target binding sites located in untranslated regions of VGAM355 host target RNA, herein designated VGAM HOST TARGET RNA. This complementary binding is due to the fact that the nucleotide sequence of VGAM355 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM355 RNA, herein designated VGAM RNA, may have a different number of host target binding sites in untranslated regions of a VGAM355 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM355 RNA, herein designated VGAM RNA, to host target binding sites on VGAM355 host target RNA, herein designated VGAM HOST TARGET RNA, such as BINDING SITE I, BINDING SITE II and BINDING SITE III, inhibits translation of VGAM355 host target RNA into VGAM355 host target protein, herein designated VGAM HOST TARGET PROTEIN. VGAM host target protein is therefore outlined by a broken line.

It is appreciated that VGAM355 host target gene, herein designated VGAM HOST TARGET GENE, in fact represents a plurality of VGAM355 host target genes. The mRNA of each one of this plurality of VGAM355 host target genes comprises one or more host target binding sites, each having a nucleotide sequence which is at least partly complementary to VGAM355 RNA, herein designated VGAM RNA, and which when bound by VGAM355 RNA causes inhibition of translation of respective one or more VGAM355 host target proteins.

It is further appreciated by one skilled in the art that the mode of translational inhibition illustrated by FIG. 1 with specific reference to translational inhibition exerted by VGAM355 gene, herein designated VGAM GENE, on one or more VGAM355 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM355 is inhibition of expression of host target genes, as part of a novel viral mechanism of attacking a host. Accordingly, utilities of VGAM355 include diagnosis, prevention and treatment of viral infection by Vaccinia Virus. Specific functions, and accordingly utilities, of VGAM355 correlate with, and may be deduced from, the identity of the host target genes which VGAM355 binds and inhibits, and the function of these host target genes, as elaborated hereinbelow.

Nucleotide sequences of the VGAM355 precursor RNA, herein designated VGAM PRECURSOR RNA, and of the ‘diced’ VGAM355 RNA, herein designated VGAM RNA, and a schematic representation of the secondary folding of VGAM355 folded precursor RNA, herein designated VGAM FOLDED PRECURSOR RNA, of VGAM355 are further described hereinbelow with reference to Table 1.

Nucleotide sequences of host target binding sites, such as BINDING SITE-I, BINDING SITE-II and BINDING SITE-III of FIG. 1, found on VGAM355 host target RNA, and schematic representation of the complementarity of each of these host target binding sites to VGAM355 RNA, herein designated VGAM RNA, are described hereinbelow with reference to Table 2.

As mentioned hereinabove with reference to FIG. 1, a function of VGAM355 gene, herein designated VGAM is inhibition of expression of VGAM355 target genes. It is appreciated that specific functions, and accordingly utilities, of VGAM355 correlate with, and may be deduced from, the identity of the target genes which VGAM355 binds and inhibits, and the function of these target genes, as elaborated hereinbelow.

AXIN1 Up-regulated 1 (AXUD1, Accession NM_{—}033027) is a VGAM355 host target gene. AXUD1 BINDING SITE is HOST TARGET binding site found in the 3′ untranslated region of mRNA encoded by AXUD1, corresponding to a HOST TARGET binding site such as BINDING SITE I, BINDING SITE II or BINDING SITE III. Table 2 illustrates the complementarity of the nucleotide sequences of AXUD1 BINDING SITE, designated SEQ ID:2309, to the nucleotide sequence of VGAM355 RNA, herein designated VGAM RNA, also designated SEQ ID:690.

A function of VGAM355 is therefore inhibition of AXIN1 Up-regulated 1 (AXUD1, Accession NM_{—}033027). Accordingly, utilities of VGAM355 include diagnosis, prevention and treatment of diseases and clinical conditions associated with AXUD1. Bassoon (presynaptic cytomatrix protein) (BSN, Accession NM_{—}003458) is another VGAM355 host target gene. BSN BINDING SITE is HOST TARGET binding site found in the 3′ untranslated region of mRNA encoded by BSN, corresponding to a HOST TARGET binding site such as BINDING SITE I, BINDING SITE II or BINDING SITE III. Table 2 illustrates the complementarity of the nucleotide sequences of BSN BINDING SITE, designated SEQ ID: 1024, to the nucleotide sequence of VGAM355 RNA, herein designated VGAM RNA, also designated SEQ ID:690.

Another function of VGAM355 is therefore inhibition of Bassoon (presynaptic cytomatrix protein) (BSN, Accession NM_{—}003458), a gene which may be involved in cytomatrix organization at the site of neurotransmitter release. Accordingly, utilities of VGAM355 include diagnosis, prevention and treatment of diseases and clinical conditions associated with BSN. The function of BSN and its association with various diseases and clinical conditions, has been established by previous studies, as described hereinabove with reference to VGAM48. Dihydropyrimidinase-like 2 (DPYSL2, Accession NM_{—}001386) is another VGAM355 host target gene. DPYSL2 BINDING SITE is HOST TARGET binding site found in the 3′ untranslated region of mRNA encoded by DPYSL2, corresponding to a HOST TARGET binding site such as BINDING SITE I, BINDING SITE II or BINDING SITE III. Table 2 illustrates the complementarity of the nucleotide sequences of DPYSL2 BINDING SITE, designated SEQ ID:822, to the nucleotide sequence of VGAM355 RNA, herein designated VGAM RNA, also designated SEQ ID:690.

Another function of VGAM355 is therefore inhibition of Dihydropyrimidinase-like 2 (DPYSL2, Accession NM_{—}001386), a gene which is a member of the dihydropyrimidinase family. Accordingly, utilities of VGAM355 include diagnosis, prevention and treatment of diseases and clinical conditions associated with DPYSL2. The function of DPYSL2 and its association with various diseases and clinical conditions, has been established by previous studies, as described hereinabove with reference to VGAM48. Epithelial V-like Antigen 1 (EVA1, Accession NM_{—}005797) is another VGAM355 host target gene. EVA1 BINDING SITE is HOST TARGET binding site found in the 3′ untranslated region of mRNA encoded by EVA1, corresponding to a HOST TARGET binding site such as BINDING SITE I, BINDING SITE II or BINDING SITE III. Table 2 illustrates the complementarity of the nucleotide sequences of EVA1 BINDING SITE, designated SEQ ID:1250, to the nucleotide sequence of VGAM355 RNA, herein designated VGAM RNA, also designated SEQ ID:690.

Another function of VGAM355 is therefore inhibition of Epithelial V-like Antigen 1 (EVA1, Accession NM_{—}005797). Accordingly, utilities of VGAM355 include diagnosis, prevention and treatment of diseases and clinical conditions associated with EVA1. Parathymosin (PTMS, Accession NM_{—}002824) is another VGAM355 host target gene. PTMS BINDING SITE is HOST TARGET binding site found in the 3′ untranslated region of mRNA encoded by PTMS, corresponding to a HOST TARGET binding site such as BINDING SITE I, BINDING SITE II or BINDING SITE III. Table 2 illustrates the complementarity of the nucleotide sequences of PTMS BINDING SITE, designated SEQ ID:959, to the nucleotide sequence of VGAM355 RNA, herein designated VGAM RNA, also designated SEQ ID:690.

Another function of VGAM355 is therefore inhibition of Parathymosin (PTMS, Accession NM_{—}002824), a gene which is involved in the regulation of cellular immunity. Accordingly, utilities of VGAM355 include diagnosis, prevention and treatment of diseases and clinical conditions associated with PTMS. The function of PTMS and its association with various diseases and clinical conditions, has been established by previous studies, as described hereinabove with reference to VGAM48. Selectin E (endothelial adhesion molecule 1) (SELE, Accession NM_{—}000450) is another VGAM355 host target gene. SELE BINDING SITE is HOST TARGET binding site found in the 3′ untranslated region of mRNA encoded by SELE, corresponding to a HOST TARGET binding site such as BINDING SITE I, BINDING SITE II or BINDING SITE III. Table 2 illustrates the complementarity of the nucleotide sequences of SELE BINDING SITE, designated SEQ ID:748, to the nucleotide sequence of VGAM355 RNA, herein designated VGAM RNA, also designated SEQ ID:690.

Another function of VGAM355 is therefore inhibition of Selectin E (endothelial adhesion molecule 1) (SELE, Accession NM_{—}000450), a gene which expressed on cytokine induced endothelial cells and mediates their binding to leukocytes. Accordingly, utilities of VGAM355 include diagnosis, prevention and treatment of diseases and clinical conditions associated with SELE. The function of SELE and its association with various diseases and clinical conditions, has been established by previous studies, as described hereinabove with reference to VGAM15. Solute Carrier Family 7 (cationic amino acid transporter, y+ system), Member 6 (SLC7A6, Accession NM_{—}003983) is another VGAM355 host target gene. SLC7A6 BINDING SITE is HOST TARGET binding site found in the 3′ untranslated region of mRNA encoded by SLC7A6, corresponding to a HOST TARGET binding site such as BINDING SITE I, BINDING SITE II or BINDING SITE III. Table 2 illustrates the complementarity of the nucleotide sequences of SLC7A6 BINDING SITE, designated SEQ ID: 1076, to the nucleotide sequence of VGAM355 RNA, herein designated VGAM RNA, also designated SEQ ID:690.

Another function of VGAM355 is therefore inhibition of Solute Carrier Family 7 (cationic amino acid transporter, y+ system), Member 6 (SLC7A6, Accession NM_{—}003983), a gene which is involved in mediating amino acid transport. Accordingly, utilities of VGAM355 include diagnosis, prevention and treatment of diseases and clinical conditions associated with SLC7A6. The function of SLC7A6 and its association with various diseases and clinical conditions, has been established by previous studies, as described hereinabove with reference to VGAM48. Transforming Growth Factor, Beta Receptor II (70/80 kDa) (TGFBR2, Accession NM_{—}003242) is another VGAM355 host target gene. TGFBR2 BINDING SITE is HOST TARGET binding site found in the 5′ untranslated region of mRNA encoded by TGFBR2, corresponding to a HOST TARGET binding site such as BINDING SITE I, BINDING SITE II or BINDING SITE III. Table 2 illustrates the complementarity of the nucleotide sequences of TGFBR2 BINDING SITE, designated SEQ ID:1003, to the nucleotide sequence of VGAM355 RNA, herein designated VGAM RNA, also designated SEQ ID:690.

Another function of VGAM355 is therefore inhibition of Transforming Growth Factor, Beta Receptor II (70/80 kDa) (TGFBR2, Accession NM_{—}003242). Accordingly, utilities of VGAM355 include diagnosis, prevention and treatment of diseases and clinical conditions associated with TGFBR2. DKFZP586N0721 (Accession NM_{—}015400) is another VGAM355 host target gene. DKFZP586N0721 BINDING SITE is HOST TARGET binding site found in the 5′ untranslated region of mRNA encoded by DKFZP586N0721, corresponding to a HOST TARGET binding site such as BINDING SITE I, BINDING SITE II or BINDING SITE III. Table 2 illustrates the complementarity of the nucleotide sequences of DKFZP586N0721 BINDING SITE, designated SEQ ID: 1627, to the nucleotide sequence of VGAM355 RNA, herein designated VGAM RNA, also designated SEQ ID:690.

Another function of VGAM355 is therefore inhibition of DKFZP586N0721 (Accession NM_{—}015400). Accordingly, utilities of VGAM355 include diagnosis, prevention and treatment of diseases and clinical conditions associated with DKFZP586N0721. DKFZp761J139 (Accession NM_{—}032280) is another VGAM355 host target gene. DKFZp761J139 BINDING SITE is HOST TARGET binding site found in the 5′ untranslated region of mRNA encoded by DKFZp761J139, corresponding to a HOST TARGET binding site such as BINDING SITE I, BINDING SITE II or BINDING SITE III. Table 2 illustrates the complementarity of the nucleotide sequences of DKFZp761J139 BINDING SITE, designated SEQ ID:2242, to the nucleotide sequence of VGAM355 RNA, herein designated VGAM RNA, also designated SEQ ID:690.

Another function of VGAM355 is therefore inhibition of DKFZp761J139 (Accession NM_{—}032280). Accordingly, utilities of VGAM355 include diagnosis, prevention and treatment of diseases and clinical conditions associated with DKFZp761J139. FLJ10468 (Accession NM_{—}018101) is another VGAM355 host target gene. FLJ10468 BINDING SITE is HOST TARGET binding site found in the 3′ untranslated region of mRNA encoded by FLJ10468, corresponding to a HOST TARGET binding site such as BINDING SITE I, BINDING SITE II or BINDING SITE III. Table 2 illustrates the complementarity of the nucleotide sequences of FLJ10468 BINDING SITE, designated SEQ ID:1779, to the nucleotide sequence of VGAM355 RNA, herein designated VGAM RNA, also designated SEQ ID:690.

Another function of VGAM355 is therefore inhibition of FLJ10468 (Accession NM_{—}018101). Accordingly, utilities of VGAM355 include diagnosis, prevention and treatment of diseases and clinical conditions associated with FLJ10468. FLJ20485 (Accession NM_{—}019042) is another VGAM355 host target gene. FLJ20485 BINDING SITE is HOST TARGET binding site found in the 3′ untranslated region of mRNA encoded by FLJ20485, corresponding to a HOST TARGET binding site such as BINDING SITE I, BINDING SITE II or BINDING SITE III. Table 2 illustrates the complementarity of the nucleotide sequences of FLJ20485 BINDING SITE, designated SEQ ID:1874, to the nucleotide sequence of VGAM355 RNA, herein designated VGAM RNA, also designated SEQ ID:690.

Another function of VGAM355 is therefore inhibition of FLJ20485 (Accession NM_{—}019042). Accordingly, utilities of VGAM355 include diagnosis, prevention and treatment of diseases and clinical conditions associated with FLJ20485. H2B Histone Family, Member Q (H2BFQ, Accession XM_{—}001940) is another VGAM355 host target gene. H2BFQ BINDING SITE is HOST TARGET binding site found in the 3′ untranslated region of mRNA encoded by H2BFQ, corresponding to a HOST TARGET binding site such as BINDING SITE I, BINDING SITE II or BINDING SITE III. Table 2 illustrates the complementarity of the nucleotide sequences of H2BFQ BINDING SITE, designated SEQ ID:2523, to the nucleotide sequence of VGAM355 RNA, herein designated VGAM RNA, also designated SEQ ID:690.

Another function of VGAM355 is therefore inhibition of H2B Histone Family, Member Q (H2BFQ, Accession XM_{—}001940). Accordingly, utilities of VGAM355 include diagnosis, prevention and treatment of diseases and clinical conditions associated with H2BFQ. KIAA0077 (Accession XM_{—}040158) is another VGAM355 host target gene. KIAA0077 BINDING SITE is HOST TARGET binding site found in the 3′ untranslated region of mRNA encoded by KIAA0077, corresponding to a HOST TARGET binding site such as BINDING SITE I, BINDING SITE II or BINDING SITE III. Table 2 illustrates the complementarity of the nucleotide sequences of KIAA0077 BINDING SITE, designated SEQ ID:2774, to the nucleotide sequence of VGAM355 RNA, herein designated VGAM RNA, also designated SEQ ID:690.

Another function of VGAM355 is therefore inhibition of KIAA0077 (Accession XM_{—}040158). Accordingly, utilities of VGAM355 include diagnosis, prevention and treatment of diseases and clinical conditions associated with KIAA0077. KIAA0295 (Accession XM_{—}042833) is another VGAM355 host target gene. KIAA0295 BINDING SITE is HOST TARGET binding site found in the 3′ untranslated region of mRNA encoded by KIAA0295, corresponding to a HOST TARGET binding site such as BINDING SITE I, BINDING SITE II or BINDING SITE III. Table 2 illustrates the complementarity of the nucleotide sequences of KIAA0295 BINDING SITE, designated SEQ ID:2807, to the nucleotide sequence of VGAM355 RNA, herein designated VGAM RNA, also designated SEQ ID:690.

Another function of VGAM355 is therefore inhibition of KIAA0295 (Accession XM_{—}042833). Accordingly, utilities of VGAM355 include diagnosis, prevention and treatment of diseases and clinical conditions associated with KIAA0295. KIAA0828 (Accession XM_{—}088105) is another VGAM355 host target gene. KIAA0828 BINDING SITE is HOST TARGET binding site found in the 3′ untranslated region of mRNA encoded by KIAA0828, corresponding to a HOST TARGET binding site such as BINDING SITE I, BINDING SITE II or BINDING SITE III. Table 2 illustrates the complementarity of the nucleotide sequences of KIAA0828 BINDING SITE, designated SEQ ID:3195, to the nucleotide sequence of VGAM355 RNA, herein designated VGAM RNA, also designated SEQ ID:690.

Another function of VGAM355 is therefore inhibition of KIAA0828 (Accession XM_{—}088105). Accordingly, utilities of VGAM355 include diagnosis, prevention and treatment of diseases and clinical conditions associated with KIAA0828. KIAA1203 (Accession XM_{—}049683) is another VGAM355 host target gene. KIAA1203 BINDING SITE is HOST TARGET binding site found in the 3′ untranslated region of mRNA encoded by KIAA1203, corresponding to a HOST TARGET binding site such as BINDING SITE I, BINDING SITE II or BINDING SITE III. Table 2 illustrates the complementarity of the nucleotide sequences of KIAA1203 BINDING SITE, designated SEQ ID:2921, to the nucleotide sequence of VGAM355 RNA, herein designated VGAM RNA, also designated SEQ ID:690.

Another function of VGAM355 is therefore inhibition of KIAA1203 (Accession XM_{—}049683). Accordingly, utilities of VGAM355 include diagnosis, prevention and treatment of diseases and clinical conditions associated with KIAA1203. Oxysterol Binding Protein-like 8 (OSBPL8, Accession NM_{—}020841) is another VGAM355 host target gene. OSBPL8 BINDING SITE is HOST TARGET binding site found in the 3′ untranslated region of mRNA encoded by OSBPL8, corresponding to a HOST TARGET binding site such as BINDING SITE I, BINDING SITE II or BINDING SITE III. Table 2 illustrates the complementarity of the nucleotide sequences of OSBPL8 BINDING SITE, designated SEQ ID:1928, to the nucleotide sequence of VGAM355 RNA, herein designated VGAM RNA, also designated SEQ ID:690.

Another function of VGAM355 is therefore inhibition of Oxysterol Binding Protein-like 8 (OSBPL8, Accession NM_{—}020841). Accordingly, utilities of VGAM355 include diagnosis, prevention and treatment of diseases and clinical conditions associated with OSBPL8. p21(CDKN1A)-activated Kinase 6 (PAK6, Accession NM_{—}020168) is another VGAM355 host target gene. PAK6 BINDING SITE is HOST TARGET binding site found in the 3′ untranslated region of mRNA encoded by PAK6, corresponding to a HOST TARGET binding site such as BINDING SITE I, BINDING SITE II or BINDING SITE III. Table 2 illustrates the complementarity of the nucleotide sequences of PAK6 BINDING SITE, designated SEQ ID:1897, to the nucleotide sequence of VGAM355 RNA, herein designated VGAM RNA, also designated SEQ ID:690.

Another function of VGAM355 is therefore inhibition of p21(CDKN1A)-activated Kinase 6 (PAK6, Accession NM_{—}020168). Accordingly, utilities of VGAM355 include diagnosis, prevention and treatment of diseases and clinical conditions associated with PAK6. Polymerase (RNA) III (DNA directed) Polypeptide F, 39 KDa (POLR3F, Accession XM_{—}009639) is another VGAM355 host target gene. POLR3F BINDING SITE is HOST TARGET binding site found in the 3′ untranslated region of mRNA encoded by POLR3F, corresponding to a HOST TARGET binding site such as BINDING SITE I, BINDING SITE II or BINDING SITE III. Table 2 illustrates the complementarity of the nucleotide sequences of POLR3F BINDING SITE, designated SEQ ID:2547, to the nucleotide sequence of VGAM355 RNA, herein designated VGAM RNA, also designated SEQ ID:690.

Another function of VGAM355 is therefore inhibition of Polymerase (RNA) III (DNA directed) Polypeptide F, 39 KDa (POLR3F, Accession XM_{—}009639). Accordingly, utilities of VGAM355 include diagnosis, prevention and treatment of diseases and clinical conditions associated with POLR3F. Ring Finger Protein 11 (RNF11, Accession NM_{—}014372) is another VGAM355 host target gene. RNF11 BINDING SITE is HOST TARGET binding site found in the 3′ untranslated region of mRNA encoded by RNF11, corresponding to a HOST TARGET binding site such as BINDING SITE I, BINDING SITE II or BINDING SITE III. Table 2 illustrates the complementarity of the nucleotide sequences of RNF11 BINDING SITE, designated SEQ ID: 1495, to the nucleotide sequence of VGAM355 RNA, herein designated VGAM RNA, also designated SEQ ID:690.

Another function of VGAM355 is therefore inhibition of Ring Finger Protein 11 (RNF11, Accession NM_{—}014372). Accordingly, utilities of VGAM355 include diagnosis, prevention and treatment of diseases and clinical conditions associated with RNF11. Solute Carrier Family 16 (monocarboxylic acid transporters), Member 6 (SLC16A6, Accession NM_{—}004694) is another VGAM355 host target gene. SLC16A6 BINDING SITE is HOST TARGET binding site found in the 5′ untranslated region of mRNA encoded by SLC16A6, corresponding to a HOST TARGET binding site such as BINDING SITE I, BINDING SITE II or BINDING SITE III. Table 2 illustrates the complementarity of the nucleotide sequences of SLC16A6 BINDING SITE, designated SEQ ID:1143, to the nucleotide sequence of VGAM355 RNA, herein designated VGAM RNA, also designated SEQ ID:690.

Another function of VGAM355 is therefore inhibition of Solute Carrier Family 16 (monocarboxylic acid transporters), Member 6 (SLC16A6, Accession NM_{—}004694). Accordingly, utilities of VGAM355 include diagnosis, prevention and treatment of diseases and clinical conditions associated with SLC16A6. LOC144321 (Accession XM_{—}096578) is another VGAM355 host target gene. LOC144321 BINDING SITE is HOST TARGET binding site found in the 3′ untranslated region of mRNA encoded by LOC144321, corresponding to a HOST TARGET binding site such as BINDING SITE I, BINDING SITE II or BINDING SITE III. Table 2 illustrates the complementarity of the nucleotide sequences of LOC144321 BINDING SITE, designated SEQ ID:3267, to the nucleotide sequence of VGAM355 RNA, herein designated VGAM RNA, also designated SEQ ID:690.

Another function of VGAM355 is therefore inhibition of LOC144321 (Accession XM_{—}096578). Accordingly, utilities of VGAM355 include diagnosis, prevention and treatment of diseases and clinical conditions associated with LOC144321. LOC150951 (Accession XM_{—}097975) is another VGAM355 host target gene. LOC150951 BINDING SITE is HOST TARGET binding site found in the 5′ untranslated region of mRNA encoded by LOC150951, corresponding to a HOST TARGET binding site such as BINDING SITE I, BINDING SITE II or BINDING SITE III. Table 2 illustrates the complementarity of the nucleotide sequences of LOC150951 BINDING SITE, designated SEQ ID:3336, to the nucleotide sequence of VGAM355 RNA, herein designated VGAM RNA, also designated SEQ ID:690.

Another function of VGAM355 is therefore inhibition of LOC150951 (Accession XM_{—}097975). Accordingly, utilities of VGAM355 include diagnosis, prevention and treatment of diseases and clinical conditions associated with LOC150951. LOC199920 (Accession XM_{—}114056) is another VGAM355 host target gene. LOC199920 BINDING SITE is HOST TARGET binding site found in the 5′ untranslated region of mRNA encoded by LOC199920, corresponding to a HOST TARGET binding site such as BINDING SITE I, BINDING SITE II or BINDING SITE III. Table 2 illustrates the complementarity of the nucleotide sequences of LOC199920 BINDING SITE, designated SEQ ID:3435, to the nucleotide sequence of VGAM355 RNA, herein designated VGAM RNA, also designated SEQ ID:690.

Another function of VGAM355 is therefore inhibition of LOC199920 (Accession XM_{—}114056). Accordingly, utilities of VGAM355 include diagnosis, prevention and treatment of diseases and clinical conditions associated with LOC199920. LOC256435 (Accession XM_{—}170925) is another VGAM355 host target gene. LOC256435 BINDING SITE is HOST TARGET binding site found in the 3′ untranslated region of mRNA encoded by LOC256435, corresponding to a HOST TARGET binding site such as BINDING SITE I, BINDING SITE II or BINDING SITE III. Table 2 illustrates the complementarity of the nucleotide sequences of LOC256435 BINDING SITE, designated SEQ ID:3675, to the nucleotide sequence of VGAM355 RNA, herein designated VGAM RNA, also designated SEQ ID:690.

Another function of VGAM355 is therefore inhibition of LOC256435 (Accession XM_{—}170925). Accordingly, utilities of VGAM355 include diagnosis, prevention and treatment of diseases and clinical conditions associated with LOC256435. FIG. 1 further provides a conceptual description of a novel bioinformatically detected viral gene of the present invention, referred to here as Viral Genomic Address Messenger 356 (VGAM356) viral gene, which modulates expression of respective host target genes thereof, the function and utility of which host target genes is known in the art.

VGAM356 is a novel bioinformatically detected regulatory, non protein coding, viral micro RNA (miRNA) gene. The method by which VGAM356 was detected is described hereinabove with reference to FIGS. 1-8.

VGAM356 gene, herein designated VGAM GENE, is a viral gene contained in the genome of Vaccinia Virus. VGAM356 host target gene, herein designated VGAM HOST TARGET GENE, is a human gene contained in the human genome.

VGAM356 gene encodes a VGAM356 precursor RNA, herein designated VGAM PRECURSOR RNA. Similar to other miRNA genes, and unlike most ordinary genes, VGAM356 precursor RNA does not encode a protein. A nucleotide sequence identical or highly similar to the nucleotide sequence of VGAM356 precursor RNA is designated SEQ ID:342, and is provided hereinbelow with reference to the sequence listing part. Nucleotide sequence SEQ ID:342 is located at position 3573 relative to the genome of Vaccinia Virus.

VGAM356 precursor RNA folds onto itself, forming VGAM356 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM356 folded precursor RNA into VGAM356 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM356 RNA is designated SEQ ID:691, and is provided hereinbelow with reference to the sequence listing part.

VGAM356 host target gene, herein designated VGAM HOST TARGET GENE, encodes a corresponding messenger RNA, VGAM356 host target RNA, herein designated VGAM HOST TARGET RNA. VGAM356 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM356 RNA, herein designated VGAM RNA, binds complementarily to one or more host target binding sites located in untranslated regions of VGAM356 host target RNA, herein designated VGAM HOST TARGET RNA. This complementary binding is due to the fact that the nucleotide sequence of VGAM356 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM356 RNA, herein designated VGAM RNA, may have a different number of host target binding sites in untranslated regions of a VGAM356 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM356 RNA, herein designated VGAM RNA, to host target binding sites on VGAM356 host target RNA, herein designated VGAM HOST TARGET RNA, such as BINDING SITE I, BINDING SITE II and BINDING SITE III, inhibits translation of VGAM356 host target RNA into VGAM356 host target protein, herein designated VGAM HOST TARGET PROTEIN. VGAM host target protein is therefore outlined by a broken line.

It is appreciated that VGAM356 host target gene, herein designated VGAM HOST TARGET GENE, in fact represents a plurality of VGAM356 host target genes. The mRNA of each one of this plurality of VGAM356 host target genes comprises one or more host target binding sites, each having a nucleotide sequence which is at least partly complementary to VGAM356 RNA, herein designated VGAM RNA, and which when bound by VGAM356 RNA causes inhibition of translation of respective one or more VGAM356 host target proteins.

It is further appreciated by one skilled in the art that the mode of translational inhibition illustrated by FIG. 1 with specific reference to translational inhibition exerted by VGAM356 gene, herein designated VGAM GENE, on one or more VGAM356 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM356 is inhibition of expression of host target genes, as part of a novel viral mechanism of attacking a host. Accordingly, utilities of VGAM356 include diagnosis, prevention and treatment of viral infection by Vaccinia Virus. Specific functions, and accordingly utilities, of VGAM356 correlate with, and may be deduced from, the identity of the host target genes which VGAM356 binds and inhibits, and the function of these host target genes, as elaborated hereinbelow.

Nucleotide sequences of the VGAM356 precursor RNA, herein designated VGAM PRECURSOR RNA, and of the ‘diced’ VGAM356 RNA, herein designated VGAM RNA, and a schematic representation of the secondary folding of VGAM356 folded precursor RNA, herein designated VGAM FOLDED PRECURSOR RNA, of VGAM356 are further described hereinbelow with reference to Table 1.

Nucleotide sequences of host target binding sites, such as BINDING SITE-I, BINDING SITE-II and BINDING SITE-III of FIG. 1, found on VGAM356 host target RNA, and schematic representation of the complementarity of each of these host target binding sites to VGAM356 RNA, herein designated VGAM RNA, are described hereinbelow with reference to Table 2.

As mentioned hereinabove with reference to FIG. 1, a function of VGAM356 gene, herein designated VGAM is inhibition of expression of VGAM356 target genes. It is appreciated that specific functions, and accordingly utilities, of VGAM356 correlate with, and may be deduced from, the identity of the target genes which VGAM356 binds and inhibits, and the function of these target genes, as elaborated hereinbelow.

A Disintegrin-like and Metalloprotease (reprolysin type) with Thrombospondin Type 1 Motif, 5 (aggrecanase-2) (ADAMTS5, Accession NM_{—}007038) is a VGAM356 host target gene. ADAMTS5 BINDING SITE is HOST TARGET binding site found in the 3′ untranslated region of mRNA encoded by ADAMTS5, corresponding to a HOST TARGET binding site such as BINDING SITE I, BINDING SITE II or BINDING SITE III. Table 2 illustrates the complementarity of the nucleotide sequences of ADAMTS5 BINDING SITE, designated SEQ ID:1352, to the nucleotide sequence of VGAM356 RNA, herein designated VGAM RNA, also designated SEQ ID:691.

A function of VGAM356 is therefore inhibition of A Disintegrin-like and Metalloprotease (reprolysin type) with Thrombospondin Type 1 Motif, 5 (aggrecanase-2) (ADAMTS5, Accession NM_{—}007038), a gene which cleaves aggrecan, a cartilage proteoglycan, and may be involved in its turnover. Accordingly, utilities of VGAM356 include diagnosis, prevention and treatment of diseases and clinical conditions associated with ADAMTS5. The function of ADAMTS5 and its association with various diseases and clinical conditions, has been established by previous studies, as described hereinabove with reference to VGAM49. Adenosine Deaminase, TRNA-specific 1 (ADAT1, Accession NM_{—}012091) is another VGAM356 host target gene. ADAT1 BINDING SITE is HOST TARGET binding site found in the 3′ untranslated region of mRNA encoded by ADAT1, corresponding to a HOST TARGET binding site such as BINDING SITE I, BINDING SITE II or BINDING SITE III. Table 2 illustrates the complementarity of the nucleotide sequences of ADAT1 BINDING SITE, designated SEQ ID:1404, to the nucleotide sequence of VGAM356 RNA, herein designated VGAM RNA, also designated SEQ ID:691.

Another function of VGAM356 is therefore inhibition of Adenosine Deaminase, TRNA-specific 1 (ADAT1, Accession NM_{—}012091), a gene which TRNA-specific adenosine deaminase; deaminates A(37) in the anticodon loop of tRNA(Ala). Accordingly, utilities of VGAM356 include diagnosis, prevention and treatment of diseases and clinical conditions associated with ADAT1. The function of ADAT1 and its association with various diseases and clinical conditions, has been established by previous studies, as described hereinabove with reference to VGAM49. Adenylate Cyclase 2 (brain) (ADCY2, Accession XM_{—}036383) is another VGAM356 host target gene. ADCY2 BINDING SITE is HOST TARGET binding site found in the 3′ untranslated region of mRNA encoded by ADCY2, corresponding to a HOST TARGET binding site such as BINDING SITE I, BINDING SITE II or BINDING SITE III. Table 2 illustrates the complementarity of the nucleotide sequences of ADCY2 BINDING SITE, designated SEQ ID:2711, to the nucleotide sequence of VGAM356 RNA, herein designated VGAM RNA, also designated SEQ ID:691.

Another function of VGAM356 is therefore inhibition of Adenylate Cyclase 2 (brain) (ADCY2, Accession XM_{—}036383), a gene which Adenylate cyclase (type 2), an ATP-pyrophosphate lyase; converts ATP to cAMP. Accordingly, utilities of VGAM356 include diagnosis, prevention and treatment of diseases and clinical conditions associated with ADCY2. The function of ADCY2 and its association with various diseases and clinical conditions, has been established by previous studies, as described hereinabove with reference to VGAM49. Aryl Hydrocarbon Receptor (AHR, Accession NM_{—}001621) is another VGAM356 host target gene. AHR BINDING SITE is HOST TARGET binding site found in the 3′ untranslated region of mRNA encoded by AHR, corresponding to a HOST TARGET binding site such as BINDING SITE I, BINDING SITE II or BINDING SITE III. Table 2 illustrates the complementarity of the nucleotide sequences of AHR BINDING SITE, designated SEQ ID:840, to the nucleotide sequence of VGAM356 RNA, herein designated VGAM RNA, also designated SEQ ID:691.

Another function of VGAM356 is therefore inhibition of Aryl Hydrocarbon Receptor (AHR, Accession NM_{—}001621), a gene which plays a role in modulating carcinogenesis through the induction of xenobiotic-metabolizing enzymes. Accordingly, utilities of VGAM356 include diagnosis, prevention and treatment of diseases and clinical conditions associated with AHR. The function of AHR and its association with various diseases and clinical conditions, has been established by previous studies, as described hereinabove with reference to VGAM49. Bassoon (presynaptic cytomatrix protein) (BSN, Accession NM_{—}003458) is another VGAM356 host target gene. BSN BINDING SITE1 and BSN BINDING SITE2 are HOST TARGET binding sites found in untranslated regions of mRNA encoded by BSN, corresponding to HOST TARGET binding sites such as BINDING SITE I, BINDING SITE II or BINDING SITE III. Table 2 illustrates the complementarity of the nucleotide sequences of BSN BINDING SITE1 and BSN BINDING SITE2, designated SEQ ID:1025 and SEQ ID: 1026 respectively, to the nucleotide sequence of VGAM356 RNA, herein designated VGAM RNA, also designated SEQ ID:691.

Another function of VGAM356 is therefore inhibition of Bassoon (presynaptic cytomatrix protein) (BSN, Accession NM_{—}003458), a gene which may be involved in cytomatrix organization at the site of neurotransmitter release. Accordingly, utilities of VGAM356 include diagnosis, prevention and treatment of diseases and clinical conditions associated with BSN. The function of BSN and its association with various diseases and clinical conditions, has been established by previous studies, as described hereinabove with reference to VGAM48. Cystic Fibrosis Transmembrane Conductance Regulator, ATP-binding Cassette (sub-family C, member 7) (CFTR, Accession NM_{—}000492) is another VGAM356 host target gene. CFTR BINDING SITE is HOST TARGET binding site found in the 3′ untranslated region of mRNA encoded by CFTR, corresponding to a HOST TARGET binding site such as BINDING SITE I, BINDING SITE II or BINDING SITE III. Table 2 illustrates the complementarity of the nucleotide sequences of CFTR BINDING SITE, designated SEQ ID:753, to the nucleotide sequence of VGAM356 RNA, herein designated VGAM RNA, also designated SEQ ID:691.

Another function of VGAM356 is therefore inhibition of Cystic Fibrosis Transmembrane Conductance Regulator, ATP-binding Cassette (sub-family C, member 7) (CFTR, Accession NM_{—}000492). Accordingly, utilities of VGAM356 include diagnosis, prevention and treatment of diseases and clinical conditions associated with CFTR. Chromodomain Helicase DNA Binding Protein 2 (CHD2, Accession NM_{—}001271) is another VGAM356 host target gene. CHD2 BINDING SITE is HOST TARGET binding site found in the 3′ untranslated region of mRNA encoded by CHD2, corresponding to a HOST TARGET binding site such as BINDING SITE I, BINDING SITE II or BINDING SITE III. Table 2 illustrates the complementarity of the nucleotide sequences of CHD2 BINDING SITE, designated SEQ ID:813, to the nucleotide sequence of VGAM356 RNA, herein designated VGAM RNA, also designated SEQ ID:691.

Another function of VGAM356 is therefore inhibition of Chromodomain Helicase DNA Binding Protein 2 (CHD2, Accession NM_{—}001271). Accordingly, utilities of VGAM356 include diagnosis, prevention and treatment of diseases and clinical conditions associated with CHD2. Cockayne Syndrome 1 (classical) (CKN1, Accession NM_{—}000082) is another VGAM356 host target gene. CKN1 BINDING SITE is HOST TARGET binding site found in the 3′ untranslated region of mRNA encoded by CKN1, corresponding to a HOST TARGET binding site such as BINDING SITE I, BINDING SITE II or BINDING SITE III. Table 2 illustrates the complementarity of the nucleotide sequences of CKN1 BINDING SITE, designated SEQ ID:703, to the nucleotide sequence of VGAM356 RNA, herein designated VGAM RNA, also designated SEQ ID:691.

Another function of VGAM356 is therefore inhibition of Cockayne Syndrome 1 (classical) (CKN1, Accession NM_{—}000082). Accordingly, utilities of VGAM356 include diagnosis, prevention and treatment of diseases and clinical conditions associated with CKN1. Cystathionase (cystathionine gamma-lyase) (CTH, Accession NM_{—}001902) is another VGAM356 host target gene. CTH BINDING SITE is HOST TARGET binding site found in the 5′ untranslated region of mRNA encoded by CTH, corresponding to a HOST TARGET binding site such as BINDING SITE I, BINDING SITE II or BINDING SITE III. Table 2 illustrates the complementarity of the nucleotide sequences of CTH BINDING SITE, designated SEQ ID:867, to the nucleotide sequence of VGAM356 RNA, herein designated VGAM RNA, also designated SEQ ID:691.

Another function of VGAM356 is therefore inhibition of Cystathionase (cystathionine gamma-lyase) (CTH, Accession NM_{—}001902), a gene which catalyzes removal of ammonia, cleaves cystathionine to yield free cys. Accordingly, utilities of VGAM356 include diagnosis, prevention and treatment of diseases and clinical conditions associated with CTH. The function of CTH and its association with various diseases and clinical conditions, has been established by previous studies, as described hereinabove with reference to VGAM49. Epithelial V-like Antigen 1 (EVA1, Accession NM_{—}005797) is another VGAM356 host target gene. EVA1 BINDING SITE is HOST TARGET binding site found in the 3′ untranslated region of mRNA encoded by EVA1, corresponding to a HOST TARGET binding site such as BINDING SITE I, BINDING SITE II or BINDING SITE III. Table 2 illustrates the complementarity of the nucleotide sequences of EVA1 BINDING SITE, designated SEQ ID:1251, to the nucleotide sequence of VGAM356 RNA, herein designated VGAM RNA, also designated SEQ ID:691.

Another function of VGAM356 is therefore inhibition of Epithelial V-like Antigen 1 (EVA1, Accession NM_{—}005797). Accordingly, utilities of VGAM356 include diagnosis, prevention and treatment of diseases and clinical conditions associated with EVA1. F-box and Leucine-rich Repeat Protein 11 (FBXL11, Accession NM_{—}012308) is another VGAM356 host target gene. FBXL11 BINDING SITE is HOST TARGET binding site found in the 3′ untranslated region of mRNA encoded by FBXL11, corresponding to a HOST TARGET binding site such as BINDING SITE I, BINDING SITE II or BINDING SITE III. Table 2 illustrates the complementarity of the nucleotide sequences of FBXL11 BINDING SITE, designated SEQ ID: 1426, to the nucleotide sequence of VGAM356 RNA, herein designated VGAM RNA, also designated SEQ ID:691.

Another function of VGAM356 is therefore inhibition of F-box and Leucine-rich Repeat Protein 11 (FBXL11, Accession NM_{—}012308), a gene which are BTB/POZ domain-containing zinc finger proteins implicated in oncogenesis. Accordingly, utilities of VGAM356 include diagnosis, prevention and treatment of diseases and clinical conditions associated with FBXL11. The function of FBXL11 and its association with various diseases and clinical conditions, has been established by previous studies, as described hereinabove with reference to VGAM49. GATA Binding Protein 3 (GATA3, Accession NM_{—}002051) is another VGAM356 host target gene. GATA3 BINDING SITE is HOST TARGET binding site found in the 3′ untranslated region of mRNA encoded by GATA3, corresponding to a HOST TARGET binding site such as BINDING SITE I, BINDING SITE II or BINDING SITE III. Table 2 illustrates the complementarity of the nucleotide sequences of GATA3 BINDING SITE, designated SEQ ID:893, to the nucleotide sequence of VGAM356 RNA, herein designated VGAM RNA, also designated SEQ ID:691.

Another function of VGAM356 is therefore inhibition of GATA Binding Protein 3 (GATA3, Accession NM_{—}002051). Accordingly, utilities of VGAM356 include diagnosis, prevention and treatment of diseases and clinical conditions associated with GATA3. GDP Dissociation Inhibitor 2 (GDI2, Accession NM_{—}001494) is another VGAM356 host target gene. GDI2 BINDING SITE is HOST TARGET binding site found in the 5′ untranslated region of mRNA encoded by GDI2, corresponding to a HOST TARGET binding site such as BINDING SITE I, BINDING SITE II or BINDING SITE III. Table 2 illustrates the complementarity of the nucleotide sequences of GDI2 BINDING SITE, designated SEQ ID:832, to the nucleotide sequence of VGAM356 RNA, herein designated VGAM RNA, also designated SEQ ID:691.

Another function of VGAM356 is therefore inhibition of GDP Dissociation Inhibitor 2 (GDI2, Accession NM_{—}001494), a gene which regulates the gdp/gtp exchange reaction of most rab proteins by inhibiting the dissociation of gdp from them, and the subsequent binding of gtp to them. Accordingly, utilities of VGAM356 include diagnosis, prevention and treatment of diseases and clinical conditions associated with GDI2. The function of GDI2 and its association with various diseases and clinical conditions, has been established by previous studies, as described hereinabove with reference to VGAM49. Huntingtin (Huntington disease) (HD, Accession NM_{—}002111) is another VGAM356 host target gene. HD BINDING SITE is HOST TARGET binding site found in the 3′ untranslated region of mRNA encoded by HD, corresponding to a HOST TARGET binding site such as BINDING SITE I, BINDING SITE II or BINDING SITE III. Table 2 illustrates the complementarity of the nucleotide sequences of HD BINDING SITE, designated SEQ ID:899, to the nucleotide sequence of VGAM356 RNA, herein designated VGAM RNA, also designated SEQ ID:691.

Another function of VGAM356 is therefore inhibition of Huntingtin (Huntington disease) (HD, Accession NM_{—}002111). Accordingly, utilities of VGAM356 include diagnosis, prevention and treatment of diseases and clinical conditions associated with HD. 5-hydroxytryptamine (serotonin) Receptor 2C (HTR2C, Accession NM_{—}000868) is another VGAM356 host target gene. HTR2C BINDING SITE is HOST TARGET binding site found in the 3′ untranslated region of mRNA encoded by HTR2C, corresponding to a HOST TARGET binding site such as BINDING SITE I, BINDING SITE II or BINDING SITE III. Table 2 illustrates the complementarity of the nucleotide sequences of HTR2C BINDING SITE, designated SEQ ID:781, to the nucleotide sequence of VGAM356 RNA, herein designated VGAM RNA, also designated SEQ ID:691.

Another function of VGAM356 is therefore inhibition of 5-hydroxytryptamine (serotonin) Receptor 2C (HTR2C, Accession NM_{—}000868), a gene which activates phospholipase C and regulates intracellular calcium flux. Accordingly, utilities of VGAM356 include diagnosis, prevention and treatment of diseases and clinical conditions associated with HTR2C. The function of HTR2C and its association with various diseases and clinical conditions, has been established by previous studies, as described hereinabove with reference to VGAM49. LGR6 (Accession XM_{—}097508) is another VGAM356 host target gene. LGR6 BINDING SITE is HOST TARGET binding site found in the 3′ untranslated region of mRNA encoded by LGR6, corresponding to a HOST TARGET binding site such as BINDING SITE I, BINDING SITE II or BINDING SITE III. Table 2 illustrates the complementarity of the nucleotide sequences of LGR6 BINDING SITE, designated SEQ ID:3302, to the nucleotide sequence of VGAM356 RNA, herein designated VGAM RNA, also designated SEQ ID:691.

Another function of VGAM356 is therefore inhibition of LGR6 (Accession XM_{—}097508). Accordingly, utilities of VGAM356 include diagnosis, prevention and treatment of diseases and clinical conditions associated with LGR6. Low Density Lipoprotein Receptor-related Protein 8, Apolipoprotein E Receptor (LRP8, Accession NM_{—}033300) is another VGAM356 host target gene. LRP8 BINDING SITE1 and LRP8 BINDING SITE2 are HOST TARGET binding sites found in untranslated regions of mRNA encoded by LRP8, corresponding to HOST TARGET binding sites such as BINDING SITE I, BINDING SITE II or BINDING SITE III. Table 2 illustrates the complementarity of the nucleotide sequences of LRP8 BINDING SITE1 and LRP8 BINDING SITE2, designated SEQ ID:2326 and SEQ ID:1139 respectively, to the nucleotide sequence of VGAM356 RNA, herein designated VGAM RNA, also designated SEQ ID:691.

Another function of VGAM356 is therefore inhibition of Low Density Lipoprotein Receptor-related Protein 8, Apolipoprotein E Receptor (LRP8, Accession NM_{—}033300), a gene which binds vldl and transports it into cells by endocytosis. Accordingly, utilities of VGAM356 include diagnosis, prevention and treatment of diseases and clinical conditions associated with LRP8. The function of LRP8 and its association with various diseases and clinical conditions, has been established by previous studies, as described hereinabove with reference to VGAM49. Macrophage Scavenger Receptor 1 (MSR1, Accession NM_{—}002445) is another VGAM356 host target gene. MSR1 BINDING SITE is HOST TARGET binding site found in the 3′ untranslated region of mRNA encoded by MSR1, corresponding to a HOST TARGET binding site such as BINDING SITE I, BINDING SITE II or BINDING SITE III. Table 2 illustrates the complementarity of the nucleotide sequences of MSR1 BINDING SITE, designated SEQ ID:929, to the nucleotide sequence of VGAM356 RNA, herein designated VGAM RNA, also designated SEQ ID:691.

Another function of VGAM356 is therefore inhibition of Macrophage Scavenger Receptor 1 (MSR1, Accession NM_{—}002445), a gene which plays a role in endocytosis of macromolecules. Accordingly, utilities of VGAM356 include diagnosis, prevention and treatment of diseases and clinical conditions associated with MSR1. The function of MSR1 and its association with various diseases and clinical conditions, has been established by previous studies, as described hereinabove with reference to VGAM49. Proto-cadherin 11 X-linked (PCDH11X, Accession NM_{—}032968) is another VGAM356 host target gene. PCDH11X BINDING SITE1 and PCDH11X BINDING SITE2 are HOST TARGET binding sites found in untranslated regions of mRNA encoded by PCDH11X, corresponding to HOST TARGET binding sites such as BINDING SITE I, BINDING SITE II or BINDING SITE III. Table 2 illustrates the complementarity of the nucleotide sequences of PCDH11X BINDING SITE1 and PCDH11X BINDING SITE2, designated SEQ ID:2299 and SEQ ID:2301 respectively, to the nucleotide sequence of VGAM356 RNA, herein designated VGAM RNA, also designated SEQ ID:691.

Another function of VGAM356 is therefore inhibition of Proto-cadherin 11 X-linked (PCDH11X, Accession NM_{—}032968), a gene which is thought to play a fundamental role in cell-cell recognition essential for the segmental development and function of the central nervous system. Accordingly, utilities of VGAM356 include diagnosis, prevention and treatment of diseases and clinical conditions associated with PCDH11X. The function of PCDH11X and its association with various diseases and clinical conditions, has been established by previous studies, as described hereinabove with reference to VGAM49. Protein-L-isoaspartate (D-aspartate) O-methyltransferase (PCMT1, Accession NM_{—}005389) is another VGAM356 host target gene. PCMT1 BINDING SITE is HOST TARGET binding site found in the 3′ untranslated region of mRNA encoded by PCMT1, corresponding to a HOST TARGET binding site such as BINDING SITE I, BINDING SITE II or BINDING SITE III. Table 2 illustrates the complementarity of the nucleotide sequences of PCMT1 BINDING SITE, designated SEQ ID:1205, to the nucleotide sequence of VGAM356 RNA, herein designated VGAM RNA, also designated SEQ ID:691.

Another function of VGAM356 is therefore inhibition of Protein-L-isoaspartate (D-aspartate) O-methyltransferase (PCMT1, Accession NM_{—}005389), a gene which catalyzes the methyl esterification of 1-isoaspartyl and d-aspartyl residues in peptides and proteins. Accordingly, utilities of VGAM356 include diagnosis, prevention and treatment of diseases and clinical conditions associated with PCMT1. The function of PCMT1 and its association with various diseases and clinical conditions, has been established by previous studies, as described hereinabove with reference to VGAM49. Proliferating Cell Nuclear Antigen (PCNA, Accession NM_{—}002592) is another VGAM356 host target gene. PCNA BINDING SITE is HOST TARGET binding site found in the 3′ untranslated region of mRNA encoded by PCNA, corresponding to a HOST TARGET binding site such as BINDING SITE I, BINDING SITE II or BINDING SITE III. Table 2 illustrates the complementarity of the nucleotide sequences of PCNA BINDING SITE, designated SEQ ID:939, to the nucleotide sequence of VGAM356 RNA, herein designated VGAM RNA, also designated SEQ ID:691.

Another function of VGAM356 is therefore inhibition of Proliferating Cell Nuclear Antigen (PCNA, Accession NM_{—}002592), a gene which is involved in the control of eukaryotic dna replication by increasing the polymerase's processibility during elongation of the leading strand. Accordingly, utilities of VGAM356 include diagnosis, prevention and treatment of diseases and clinical conditions associated with PCNA. The function of PCNA and its association with various diseases and clinical conditions, has been established by previous studies, as described hereinabove with reference to VGAM49. Proline Arginine-rich End Leucine-rich Repeat Protein (PRELP, Accession NM_{—}002725) is another VGAM356 host target gene. PRELP BINDING SITE is HOST TARGET binding site found in the 3′ untranslated region of mRNA encoded by PRELP, corresponding to a HOST TARGET binding site such as BINDING SITE I, BINDING SITE II or BINDING SITE III. Table 2 illustrates the complementarity of the nucleotide sequences of PRELP BINDING SITE, designated SEQ ID:950, to the nucleotide sequence of VGAM356 RNA, herein designated VGAM RNA, also designated SEQ ID:691.

Another function of VGAM356 is therefore inhibition of Proline Arginine-rich End Leucine-rich Repeat Protein (PRELP, Accession NM_{—}002725), a gene which is a connective tissue glycoprotein of the leucine-rich-repeat (LRR) family. Accordingly, utilities of VGAM356 include diagnosis, prevention and treatment of diseases and clinical conditions associated with PRELP. The function of PRELP and its association with various diseases and clinical conditions, has been established by previous studies, as described hereinabove with reference to VGAM49. Parathymosin (PTMS, Accession NM_{—}002824) is another VGAM356 host target gene. PTMS BINDING SITE is HOST TARGET binding site found in the 3′ untranslated region of mRNA encoded by PTMS, corresponding to a HOST TARGET binding site such as BINDING SITE I, BINDING SITE II or BINDING SITE III. Table 2 illustrates the complementarity of the nucleotide sequences of PTMS BINDING SITE, designated SEQ ID:958, to the nucleotide sequence of VGAM356 RNA, herein designated VGAM RNA, also designated SEQ ID:691.

Another function of VGAM356 is therefore inhibition of Parathymosin (PTMS, Accession NM_{—}002824), a gene which is involved in the regulation of cellular immunity. Accordingly, utilities of VGAM356 include diagnosis, prevention and treatment of diseases and clinical conditions associated with PTMS. The function of PTMS and its association with various diseases and clinical conditions, has been established by previous studies, as described hereinabove with reference to VGAM48. Solute Carrier Family 4, Sodium Bicarbonate Cotransporter, Member 4 (SLC4A4, Accession NM_{—}003759) is another VGAM356 host target gene. SLC4A4 BINDING SITE is HOST TARGET binding site found in the 3′ untranslated region of mRNA encoded by SLC4A4, corresponding to a HOST TARGET binding site such as BINDING SITE I, BINDING SITE II or BINDING SITE III. Table 2 illustrates the complementarity of the nucleotide sequences of SLC4A4 BINDING SITE, designated SEQ ID:1054, to the nucleotide sequence of VGAM356 RNA, herein designated VGAM RNA, also designated SEQ ID:691.

Another function of VGAM356 is therefore inhibition of Solute Carrier Family 4, Sodium Bicarbonate Cotransporter, Member 4 (SLC4A4, Accession NM_{—}003759), a gene which is a sodium bicarbonate cotransporter. Accordingly, utilities of VGAM356 include diagnosis, prevention and treatment of diseases and clinical conditions associated with SLC4A4. The function of SLC4A4 and its association with various diseases and clinical conditions, has been established by previous studies, as described hereinabove with reference to VGAM49. Trichorhinophalangeal Syndrome I (TRPS1, Accession NM_{—}014112) is another VGAM356 host target gene. TRPS1 BINDING SITE is HOST TARGET binding site found in the 5′ untranslated region of mRNA encoded by TRPS1, corresponding to a HOST TARGET binding site such as BINDING SITE I, BINDING SITE II or BINDING SITE III. Table 2 illustrates the complementarity of the nucleotide sequences of TRPS1 BINDING SITE, designated SEQ ID: 1471, to the nucleotide sequence of VGAM356 RNA, herein designated VGAM RNA, also designated SEQ ID:691.

Another function of VGAM356 is therefore inhibition of Trichorhinophalangeal Syndrome I (TRPS1, Accession NM_{—}014112), a gene which may function as a transcriptional activator protein. Accordingly, utilities of VGAM356 include diagnosis, prevention and treatment of diseases and clinical conditions associated with TRPS1. The function of TRPS1 and its association with various diseases and clinical conditions, has been established by previous studies, as described hereinabove with reference to VGAM49. ARS2 (Accession NM_{—}015908) is another VGAM356 host target gene. ARS2 BINDING SITE is HOST TARGET binding site found in the 5′ untranslated region of mRNA encoded by ARS2, corresponding to a HOST TARGET binding site such as BINDING SITE I, BINDING SITE II or BINDING SITE III. Table 2 illustrates the complementarity of the nucleotide sequences of ARS2 BINDING SITE, designated SEQ ID: 1650, to the nucleotide sequence of VGAM356 RNA, herein designated VGAM RNA, also designated SEQ ID:691.

Another function of VGAM356 is therefore inhibition of ARS2 (Accession NM_{—}015908). Accordingly, utilities of VGAM356 include diagnosis, prevention and treatment of diseases and clinical conditions associated with ARS2. Bromodomain Containing 4 (BRD4, Accession NM_{—}058243) is another VGAM356 host target gene. BRD4 BINDING SITE is HOST TARGET binding site found in the 3′ untranslated region of mRNA encoded by BRD4, corresponding to a HOST TARGET binding site such as BINDING SITE I, BINDING SITE II or BINDING SITE III. Table 2 illustrates the complementarity of the nucleotide sequences of BRD4 BINDING SITE, designated SEQ ID:2368, to the nucleotide sequence of VGAM356 RNA, herein designated VGAM RNA, also designated SEQ ID:691.

Another function of VGAM356 is therefore inhibition of Bromodomain Containing 4 (BRD4, Accession NM_{—}058243). Accordingly, utilities of VGAM356 include diagnosis, prevention and treatment of diseases and clinical conditions associated with BRD4. CAPN13 (Accession NM_{—}144575) is another VGAM356 host target gene. CAPN13 BINDING SITE is HOST TARGET binding site found in the 3′ untranslated region of mRNA encoded by CAPN13, corresponding to a HOST TARGET binding site such as BINDING SITE I, BINDING SITE II or BINDING SITE III. Table 2 illustrates the complementarity of the nucleotide sequences of CAPN13 BINDING SITE, designated SEQ ID:2485, to the nucleotide sequence of VGAM356 RNA, herein designated VGAM RNA, also designated SEQ ID:691.

Another function of VGAM356 is therefore inhibition of CAPN13 (Accession NM_{—}144575). Accordingly, utilities of VGAM356 include diagnosis, prevention and treatment of diseases and clinical conditions associated with CAPN13. Cat Eye Syndrome Chromosome Region, Candidate 6 (CECR6, Accession NM_{—}031890) is another VGAM356 host target gene. CECR6 BINDING SITE is HOST TARGET binding site found in the 3′ untranslated region of mRNA encoded by CECR6, corresponding to a HOST TARGET binding site such as BINDING SITE I, BINDING SITE II or BINDING SITE III. Table 2 illustrates the complementarity of the nucleotide sequences of CECR6 BINDING SITE, designated SEQ ID:2218, to the nucleotide sequence of VGAM356 RNA, herein designated VGAM RNA, also designated SEQ ID:691.

Another function of VGAM356 is therefore inhibition of Cat Eye Syndrome Chromosome Region, Candidate 6 (CECR6, Accession NM_{—}031890). Accordingly, utilities of VGAM356 include diagnosis, prevention and treatment of diseases and clinical conditions associated with CECR6. DKFZP43410714 (Accession XM_{—}098247) is another VGAM356 host target gene. DKFZP43410714 BINDING SITE is HOST TARGET binding site found in the 5′ untranslated region of mRNA encoded by DKFZP43410714, corresponding to a HOST TARGET binding site such as BINDING SITE I, BINDING SITE II or BINDING SITE III. Table 2 illustrates the complementarity of the nucleotide sequences of DKFZP43410714 BINDING SITE, designated SEQ ID:3362, to the nucleotide sequence of VGAM356 RNA, herein designated VGAM RNA, also designated SEQ ID:691.

Another function of VGAM356 is therefore inhibition of DKFZP43410714 (Accession XM_{—}098247). Accordingly, utilities of VGAM356 include diagnosis, prevention and treatment of diseases and clinical conditions associated with DKFZP43410714. DKFZP564G092 (Accession NM_{—}015601) is another VGAM356 host target gene. DKFZP564G092 BINDING SITE is HOST TARGET binding site found in the 5′ untranslated region of mRNA encoded by DKFZP564G092, corresponding to a HOST TARGET binding site such as BINDING SITE I, BINDING SITE II or BINDING SITE III. Table 2 illustrates the complementarity of the nucleotide sequences of DKFZP564G092 BINDING SITE, designated SEQ ID: 1639, to the nucleotide sequence of VGAM356 RNA, herein designated VGAM RNA, also designated SEQ ID:691.

Another function of VGAM356 is therefore inhibition of DKFZP564G092 (Accession NM_{—}015601). Accordingly, utilities of VGAM356 include diagnosis, prevention and treatment of diseases and clinical conditions associated with DKFZP564G092. DKFZp564K142 (Accession NM_{—}032121) is another VGAM356 host target gene. DKFZp564K142 BINDING SITE is HOST TARGET binding site found in the 3′ untranslated region of mRNA encoded by DKFZp564K142, corresponding to a HOST TARGET binding site such as BINDING SITE I, BINDING SITE II or BINDING SITE III. Table 2 illustrates the complementarity of the nucleotide sequences of DKFZp564K142 BINDING SITE, designated SEQ ID:2231, to the nucleotide sequence of VGAM356 RNA, herein designated VGAM RNA, also designated SEQ ID:691.

Another function of VGAM356 is therefore inhibition of DKFZp564K142 (Accession NM_{—}032121). Accordingly, utilities of VGAM356 include diagnosis, prevention and treatment of diseases and clinical conditions associated with DKFZp564K142. DKFZp566D234 (Accession XM_{—}030162) is another VGAM356 host target gene. DKFZp566D234 BINDING SITE is HOST TARGET binding site found in the 3′ untranslated region of mRNA encoded by DKFZp566D234, corresponding to a HOST TARGET binding site such as BINDING SITE I, BINDING SITE II or BINDING SITE III. Table 2 illustrates the complementarity of the nucleotide sequences of DKFZp566D234 BINDING SITE, designated SEQ ID:2612, to the nucleotide sequence of VGAM356 RNA, herein designated VGAM RNA, also designated SEQ ID:691.

Another function of VGAM356 is therefore inhibition of DKFZp566D234 (Accession XM_{—}030162). Accordingly, utilities of VGAM356 include diagnosis, prevention and treatment of diseases and clinical conditions associated with DKFZp566D234. DKFZP56611024 (Accession XM_{—}046506) is another VGAM356 host target gene. DKFZP56611024 BINDING SITE is HOST TARGET binding site found in the 3′ untranslated region of mRNA encoded by DKFZP56611024, corresponding to a HOST TARGET binding site such as BINDING SITE I, BINDING SITE II or BINDING SITE III. Table 2 illustrates the complementarity of the nucleotide sequences of DKFZP56611024 BINDING SITE, designated SEQ ID:2878, to the nucleotide sequence of VGAM356 RNA, herein designated VGAM RNA, also designated SEQ ID:691.

Another function of VGAM356 is therefore inhibition of DKFZP56611024 (Accession XM_{—}046506). Accordingly, utilities of VGAM356 include diagnosis, prevention and treatment of diseases and clinical conditions associated with DKFZP56611024. EP164 (Accession XM_{—}170997) is another VGAM356 host target gene. EP164 BINDING SITE is HOST TARGET binding site found in the 3′ untranslated region of mRNA encoded by EP164, corresponding to a HOST TARGET binding site such as BINDING SITE I, BINDING SITE II or BINDING SITE III. Table 2 illustrates the complementarity of the nucleotide sequences of EP164 BINDING SITE, designated SEQ ID:3681, to the nucleotide sequence of VGAM356 RNA, herein designated VGAM RNA, also designated SEQ ID:691.

Another function of VGAM356 is therefore inhibition of EP164 (Accession XM_{—}170997). Accordingly, utilities of VGAM356 include diagnosis, prevention and treatment of diseases and clinical conditions associated with EP164. FLJ0012 (Accession XM_{—}058426) is another VGAM356 host target gene. FLJ00012 BINDING SITE is HOST TARGET binding site found in the 3′ untranslated region of mRNA encoded by FLJ00012, corresponding to a HOST TARGET binding site such as BINDING SITE I, BINDING SITE II or BINDING SITE III. Table 2 illustrates the complementarity of the nucleotide sequences of FLJ00012 BINDING SITE, designated SEQ ID:2985, to the nucleotide sequence of VGAM356 RNA, herein designated VGAM RNA, also designated SEQ ID:691.

Another function of VGAM356 is therefore inhibition of FLJ00012 (Accession XM_{—}058426). Accordingly, utilities of VGAM356 include diagnosis, prevention and treatment of diseases and clinical conditions associated with FLJ00012. FLJ10460 (Accession NM_{—}018097) is another VGAM356 host target gene. FLJ10460 BINDING SITE is HOST TARGET binding site found in the 3′ untranslated region of mRNA encoded by FLJ10460, corresponding to a HOST TARGET binding site such as BINDING SITE I, BINDING SITE II or BINDING SITE III. Table 2 illustrates the complementarity of the nucleotide sequences of FLJ10460 BINDING SITE, designated SEQ ID:1778, to the nucleotide sequence of VGAM356 RNA, herein designated VGAM RNA, also designated SEQ ID:691.

Another function of VGAM356 is therefore inhibition of FLJ10460 (Accession NM_{—}018097). Accordingly, utilities of VGAM356 include diagnosis, prevention and treatment of diseases and clinical conditions associated with FLJ10460. FLJ10687 (Accession NM_{—}018178) is another VGAM356 host target gene. FLJ10687 BINDING SITE is HOST TARGET binding site found in the 3′ untranslated region of mRNA encoded by FLJ10687, corresponding to a HOST TARGET binding site such as BINDING SITE I, BINDING SITE II or BINDING SITE III. Table 2 illustrates the complementarity of the nucleotide sequences of FLJ10687 BINDING SITE, designated SEQ ID:1793, to the nucleotide sequence of VGAM356 RNA, herein designated VGAM RNA, also designated SEQ ID:691.

Another function of VGAM356 is therefore inhibition of FLJ10687 (Accession NM_{—}018178). Accordingly, utilities of VGAM356 include diagnosis, prevention and treatment of diseases and clinical conditions associated with FLJ10687. FLJ10738 (Accession NM_{—}018199) is another VGAM356 host target gene. FLJ10738 BINDING SITE is HOST TARGET binding site found in the 3′ untranslated region of mRNA encoded by FLJ10738, corresponding to a HOST TARGET binding site such as BINDING SITE I, BINDING SITE II or BINDING SITE III. Table 2 illustrates the complementarity of the nucleotide sequences of FLJ10738 BINDING SITE, designated SEQ ID:1799, to the nucleotide sequence of VGAM356 RNA, herein designated VGAM RNA, also designated SEQ ID:691.

Another function of VGAM356 is therefore inhibition of FLJ10738 (Accession NM_{—}018199). Accordingly, utilities of VGAM356 include diagnosis, prevention and treatment of diseases and clinical conditions associated with FLJ10738. FLJ13158 (Accession NM_{—}024909) is another VGAM356 host target gene. FLJ13158 BINDING SITE is HOST TARGET binding site found in the 3′ untranslated region of mRNA encoded by FLJ13158, corresponding to a HOST TARGET binding site such as BINDING SITE I, BINDING SITE II or BINDING SITE III. Table 2 illustrates the complementarity of the nucleotide sequences of FLJ13158 BINDING SITE, designated SEQ ID:2109, to the nucleotide sequence of VGAM356 RNA, herein designated VGAM RNA, also designated SEQ ID:691.

Another function of VGAM356 is therefore inhibition of FLJ13158 (Accession NM_{—}024909). Accordingly, utilities of VGAM356 include diagnosis, prevention and treatment of diseases and clinical conditions associated with FLJ13158. FLJ20294 (Accession NM_{—}017749) is another VGAM356 host target gene. FLJ20294 BINDING SITE is HOST TARGET binding site found in the 5′ untranslated region of mRNA encoded by FLJ20294, corresponding to a HOST TARGET binding site such as BINDING SITE I, BINDING SITE II or BINDING SITE III. Table 2 illustrates the complementarity of the nucleotide sequences of FLJ20294 BINDING SITE, designated SEQ ID:1747, to the nucleotide sequence of VGAM356 RNA, herein designated VGAM RNA, also designated SEQ ID:691.

Another function of VGAM356 is therefore inhibition of FLJ20294 (Accession NM_{—}017749). Accordingly, utilities of VGAM356 include diagnosis, prevention and treatment of diseases and clinical conditions associated with FLJ20294. FLJ21657 (Accession NM_{—}022483) is another VGAM356 host target gene. FLJ21657 BINDING SITE is HOST TARGET binding site found in the 3′ untranslated region of mRNA encoded by FLJ21657, corresponding to a HOST TARGET binding site such as BINDING SITE I, BINDING SITE II or BINDING SITE III. Table 2 illustrates the complementarity of the nucleotide sequences of FLJ21657 BINDING SITE, designated SEQ ID:1991, to the nucleotide sequence of VGAM356 RNA, herein designated VGAM RNA, also designated SEQ ID:691.

Another function of VGAM356 is therefore inhibition of FLJ21657 (Accession NM_{—}022483). Accordingly, utilities of VGAM356 include diagnosis, prevention and treatment of diseases and clinical conditions associated with FLJ21657. GT650 (Accession NM_{—}052851) is another VGAM356 host target gene. GT650 BINDING SITE is HOST TARGET binding site found in the 3′ untranslated region of mRNA encoded by GT650, corresponding to a HOST TARGET binding site such as BINDING SITE I, BINDING SITE II or BINDING SITE III. Table 2 illustrates the complementarity of the nucleotide sequences of GT650 BINDING SITE, designated SEQ ID:2341, to the nucleotide sequence of VGAM356 RNA, herein designated VGAM RNA, also designated SEQ ID:691.

Another function of VGAM356 is therefore inhibition of GT650 (Accession NM_{—}052851). Accordingly, utilities of VGAM356 include diagnosis, prevention and treatment of diseases and clinical conditions associated with GT650. Hairy/enhancer-of-split Related with YRPW Motif 2 (HEY2, Accession NM_{—}012259) is another VGAM356 host target gene. HEY2 BINDING SITE is HOST TARGET binding site found in the 3′ untranslated region of mRNA encoded by HEY2, corresponding to a HOST TARGET binding site such as BINDING SITE I, BINDING SITE II or BINDING SITE III. Table 2 illustrates the complementarity of the nucleotide sequences of HEY2 BINDING SITE, designated SEQ ID: 1420, to the nucleotide sequence of VGAM356 RNA, herein designated VGAM RNA, also designated SEQ ID:691.

Another function of VGAM356 is therefore inhibition of Hairy/enhancer-of-split Related with YRPW Motif 2 (HEY2, Accession NM_{—}012259). Accordingly, utilities of VGAM356 include diagnosis, prevention and treatment of diseases and clinical conditions associated with HEY2. Hypermethylated In Cancer 2 (HIC2, Accession XM_{—}036937) is another VGAM356 host target gene. HIC2 BINDING SITE is HOST TARGET binding site found in the 3′ untranslated region of mRNA encoded by HIC2, corresponding to a HOST TARGET binding site such as BINDING SITE I, BINDING SITE II or BINDING SITE III. Table 2 illustrates the complementarity of the nucleotide sequences of HIC2 BINDING SITE, designated SEQ ID:2717, to the nucleotide sequence of VGAM356 RNA, herein designated VGAM RNA, also designated SEQ ID:691.

Another function of VGAM356 is therefore inhibition of Hypermethylated In Cancer 2 (HIC2, Accession XM_{—}036937). Accordingly, utilities of VGAM356 include diagnosis, prevention and treatment of diseases and clinical conditions associated with HIC2. HOOK3 (Accession NM_{—}032410) is another VGAM356 host target gene. HOOK3 BINDING SITE is HOST TARGET binding site found in the 3′ untranslated region of mRNA encoded by HOOK3, corresponding to a HOST TARGET binding site such as BINDING SITE I, BINDING SITE II or BINDING SITE III. Table 2 illustrates the complementarity of the nucleotide sequences of HOOK3 BINDING SITE, designated SEQ ID:2249, to the nucleotide sequence of VGAM356 RNA, herein designated VGAM RNA, also designated SEQ ID:691.

Another function of VGAM356 is therefore inhibition of HOOK3 (Accession NM_{—}032410). Accordingly, utilities of VGAM356 include diagnosis, prevention and treatment of diseases and clinical conditions associated with HOOK3. Interleukin 1 Receptor Accessory Protein-like 1 (IL1RAPL1, Accession NM_{—}014271) is another VGAM356 host target gene. IL1RAPL1 BINDING SITE is HOST TARGET binding site found in the 5′ untranslated region of mRNA encoded by IL1RAPL1, corresponding to a HOST TARGET binding site such as BINDING SITE I, BINDING SITE II or BINDING SITE III. Table 2 illustrates the complementarity of the nucleotide sequences of IL1RAPL1 BINDING SITE, designated SEQ ID: 1488, to the nucleotide sequence of VGAM356 RNA, herein designated VGAM RNA, also designated SEQ ID:691.

Another function of VGAM356 is therefore inhibition of Interleukin 1 Receptor Accessory Protein-like 1 (IL1RAPL1, Accession NM_{—}014271). Accordingly, utilities of VGAM356 include diagnosis, prevention and treatment of diseases and clinical conditions associated with IL1RAPL1. KALI (Accession NM_{—}052931) is another VGAM356 host target gene. KALI BINDING SITE is HOST TARGET binding site found in the 3′ untranslated region of mRNA encoded by KALI, corresponding to a HOST TARGET binding site such as BINDING SITE I, BINDING SITE II or BINDING SITE III. Table 2 illustrates the complementarity of the nucleotide sequences of KALI BINDING SITE, designated SEQ ID:2347, to the nucleotide sequence of VGAM356 RNA, herein designated VGAM RNA, also designated SEQ ID:691.

Another function of VGAM356 is therefore inhibition of KALI (Accession NM_{—}052931). Accordingly, utilities of VGAM356 include diagnosis, prevention and treatment of diseases and clinical conditions associated with KALI. KIAA0077 (Accession XM_{—}040158) is another VGAM356 host target gene. KIAA0077 BINDING SITE is HOST TARGET binding site found in the 3′ untranslated region of mRNA encoded by KIAA0077, corresponding to a HOST TARGET binding site such as BINDING SITE I, BINDING SITE II or BINDING SITE III. Table 2 illustrates the complementarity of the nucleotide sequences of KIAA0077 BINDING SITE, designated SEQ ID:2773, to the nucleotide sequence of VGAM356 RNA, herein designated VGAM RNA, also designated SEQ ID:691.

Another function of VGAM356 is therefore inhibition of KIAA0077 (Accession XM_{—}040158). Accordingly, utilities of VGAM356 include diagnosis, prevention and treatment of diseases and clinical conditions associated with KIAA0077. KIAA0089 (Accession XM_{—}046056) is another VGAM356 host target gene. KIAA0089 BINDING SITE is HOST TARGET binding site found in the 3′ untranslated region of mRNA encoded by KIAA0089, corresponding to a HOST TARGET binding site such as BINDING SITE I, BINDING SITE II or BINDING SITE III. Table 2 illustrates the complementarity of the nucleotide sequences of KIAA0089 BINDING SITE, designated SEQ ID:2873, to the nucleotide sequence of VGAM356 RNA, herein designated VGAM RNA, also designated SEQ ID:691.

Another function of VGAM356 is therefore inhibition of KIAA0089 (Accession XM_{—}046056). Accordingly, utilities of VGAM356 include diagnosis, prevention and treatment of diseases and clinical conditions associated with KIAA0089. KIAA0286 (Accession XM_{—}043118) is another VGAM356 host target gene. KIAA0286 BINDING SITE is HOST TARGET binding site found in the 3′ untranslated region of mRNA encoded by KIAA0286, corresponding to a HOST TARGET binding site such as BINDING SITE I, BINDING SITE II or BINDING SITE III. Table 2 illustrates the complementarity of the nucleotide sequences of KIAA0286 BINDING SITE, designated SEQ ID:2814, to the nucleotide sequence of VGAM356 RNA, herein designated VGAM RNA, also designated SEQ ID:691.

Another function of VGAM356 is therefore inhibition of KIAA0286 (Accession XM_{—}043118). Accordingly, utilities of VGAM356 include diagnosis, prevention and treatment of diseases and clinical conditions associated with KIAA0286. KIAA0295 (Accession XM_{—}042833) is another VGAM356 host target gene. KIAA0295 BINDING SITE is HOST TARGET binding site found in the 3′ untranslated region of mRNA encoded by KIAA0295, corresponding to a HOST TARGET binding site such as BINDING SITE I, BINDING SITE II or BINDING SITE III. Table 2 illustrates the complementarity of the nucleotide sequences of KIAA0295 BINDING SITE, designated SEQ ID:2808, to the nucleotide sequence of VGAM356 RNA, herein designated VGAM RNA, also designated SEQ ID:691.

Another function of VGAM356 is therefore inhibition of KIAA0295 (Accession XM_{—}042833). Accordingly, utilities of VGAM356 include diagnosis, prevention and treatment of diseases and clinical conditions associated with KIAA0295. KIAA0342 (Accession XM_{—}047357) is another VGAM356 host target gene. KIAA0342 BINDING SITE is HOST TARGET binding site found in the 3′ untranslated region of mRNA encoded by KIAA0342, corresponding to a HOST TARGET binding site such as BINDING SITE I, BINDING SITE II or BINDING SITE III. Table 2 illustrates the complementarity of the nucleotide sequences of KIAA0342 BINDING SITE, designated SEQ ID:2890, to the nucleotide sequence of VGAM356 RNA, herein designated VGAM RNA, also designated SEQ ID:691.

Another function of VGAM356 is therefore inhibition of KIAA0342 (Accession XM_{—}047357). Accordingly, utilities of VGAM356 include diagnosis, prevention and treatment of diseases and clinical conditions associated with KIAA0342. KIAA0564 (Accession XM_{—}038664) is another VGAM356 host target gene. KIAA0564 BINDING SITE is HOST TARGET binding site found in the 3′ untranslated region of mRNA encoded by KIAA0564, corresponding to a HOST TARGET binding site such as BINDING SITE I, BINDING SITE II or BINDING SITE III. Table 2 illustrates the complementarity of the nucleotide sequences of KIAA0564 BINDING SITE, designated SEQ ID:2743, to the nucleotide sequence of VGAM356 RNA, herein designated VGAM RNA, also designated SEQ ID:691.

Another function of VGAM356 is therefore inhibition of KIAA0564 (Accession XM_{—}038664). Accordingly, utilities of VGAM356 include diagnosis, prevention and treatment of diseases and clinical conditions associated with KIAA0564. KIAA0630 (Accession XM_{—}114729) is another VGAM356 host target gene. KIAA0630 BINDING SITE is HOST TARGET binding site found in the 3′ untranslated region of mRNA encoded by KIAA0630, corresponding to a HOST TARGET binding site such as BINDING SITE I, BINDING SITE II or BINDING SITE III. Table 2 illustrates the complementarity of the nucleotide sequences of KIAA0630 BINDING SITE, designated SEQ ID:3465, to the nucleotide sequence of VGAM356 RNA, herein designated VGAM RNA, also designated SEQ ID:691.

Another function of VGAM356 is therefore inhibition of KIAA0630 (Accession XM_{—}114729). Accordingly, utilities of VGAM356 include diagnosis, prevention and treatment of diseases and clinical conditions associated with KIAA0630. KIAA0662 (Accession XM_{—}088539) is another VGAM356 host target gene. KIAA0662 BINDING SITE is HOST TARGET binding site found in the 3′ untranslated region of mRNA encoded by KIAA0662, corresponding to a HOST TARGET binding site such as BINDING SITE I, BINDING SITE II or BINDING SITE III. Table 2 illustrates the complementarity of the nucleotide sequences of KIAA0662 BINDING SITE, designated SEQ ID:3215, to the nucleotide sequence of VGAM356 RNA, herein designated VGAM RNA, also designated SEQ ID:691.

Another function of VGAM356 is therefore inhibition of KIAA0662 (Accession XM_{—}088539). Accordingly, utilities of VGAM356 include diagnosis, prevention and treatment of diseases and clinical conditions associated with KIAA0662. KIAA0830 (Accession XM_{—}045759) is another VGAM356 host target gene. KIAA0830 BINDING SITE is HOST TARGET binding site found in the 3′ untranslated region of mRNA encoded by KIAA0830, corresponding to a HOST TARGET binding site such as BINDING SITE I, BINDING SITE II or BINDING SITE III. Table 2 illustrates the complementarity of the nucleotide sequences of KIAA0830 BINDING SITE, designated SEQ ID:2866, to the nucleotide sequence of VGAM356 RNA, herein designated VGAM RNA, also designated SEQ ID:691.

Another function of VGAM356 is therefore inhibition of KIAA0830 (Accession XM_{—}045759). Accordingly, utilities of VGAM356 include diagnosis, prevention and treatment of diseases and clinical conditions associated with KIAA0830. KIAA0964 (Accession NM_{—}014902) is another VGAM356 host target gene. KIAA0964 BINDING SITE is HOST TARGET binding site found in the 3′ untranslated region of mRNA encoded by KIAA0964, corresponding to a HOST TARGET binding site such as BINDING SITE I, BINDING SITE II or BINDING SITE III. Table 2 illustrates the complementarity of the nucleotide sequences of KIAA0964 BINDING SITE, designated SEQ ID:1579, to the nucleotide sequence of VGAM356 RNA, herein designated VGAM RNA, also designated SEQ ID:691.

Another function of VGAM356 is therefore inhibition of KIAA0964 (Accession NM_{—}014902). Accordingly, utilities of VGAM356 include diagnosis, prevention and treatment of diseases and clinical conditions associated with KIAA0964. KIAA1076 (Accession XM_{—}037523) is another VGAM356 host target gene. KIAA1076 BINDING SITE is HOST TARGET binding site found in the 3′ untranslated region of mRNA encoded by KIAA1076, corresponding to a HOST TARGET binding site such as BINDING SITE I, BINDING SITE II or BINDING SITE III. Table 2 illustrates the complementarity of the nucleotide sequences of KIAA1076 BINDING SITE, designated SEQ ID:2723, to the nucleotide sequence of VGAM356 RNA, herein designated VGAM RNA, also designated SEQ ID:691.

Another function of VGAM356 is therefore inhibition of KIAA1076 (Accession XM_{—}037523). Accordingly, utilities of VGAM356 include diagnosis, prevention and treatment of diseases and clinical conditions associated with KIAA1076. KIAA1181 (Accession XM_{—}043340) is another VGAM356 host target gene. KIAA1181 BINDING SITE is HOST TARGET binding site found in the 3′ untranslated region of mRNA encoded by KIAA1181, corresponding to a HOST TARGET binding site such as BINDING SITE I, BINDING SITE II or BINDING SITE III. Table 2 illustrates the complementarity of the nucleotide sequences of KIAA1181 BINDING SITE, designated SEQ ID:2817, to the nucleotide sequence of VGAM356 RNA, herein designated VGAM RNA, also designated SEQ ID:691.

Another function of VGAM356 is therefore inhibition of KIAA1181 (Accession XM_{—}043340). Accordingly, utilities of VGAM356 include diagnosis, prevention and treatment of diseases and clinical conditions associated with KIAA1181. KIAA1319 (Accession NM_{—}020770) is another VGAM356 host target gene. KIAA1319 BINDING SITE is HOST TARGET binding site found in the 3′ untranslated region of mRNA encoded by KIAA1319, corresponding to a HOST TARGET binding site such as BINDING SITE I, BINDING SITE II or BINDING SITE III. Table 2 illustrates the complementarity of the nucleotide sequences of KIAA1319 BINDING SITE, designated SEQ ID:1925, to the nucleotide sequence of VGAM356 RNA, herein designated VGAM RNA, also designated SEQ ID:691.

Another function of VGAM356 is therefore inhibition of KIAA1319 (Accession NM_{—}020770). Accordingly, utilities of VGAM356 include diagnosis, prevention and treatment of diseases and clinical conditions associated with KIAA1319. KIAA1522 (Accession XM_{—}036299) is another VGAM356 host target gene. KIAA1522 BINDING SITE is HOST TARGET binding site found in the 3′ untranslated region of mRNA encoded by KIAA1522, corresponding to a HOST TARGET binding site such as BINDING SITE I, BINDING SITE II or BINDING SITE III. Table 2 illustrates the complementarity of the nucleotide sequences of KIAA1522 BINDING SITE, designated SEQ ID:2708, to the nucleotide sequence of VGAM356 RNA, herein designated VGAM RNA, also designated SEQ ID:691.

Another function of VGAM356 is therefore inhibition of KIAA1522 (Accession XM_{—}036299). Accordingly, utilities of VGAM356 include diagnosis, prevention and treatment of diseases and clinical conditions associated with KIAA1522. KIAA1906 (Accession XM_{—}055095) is another VGAM356 host target gene. KIAA1906 BINDING SITE is HOST TARGET binding site found in the 3′ untranslated region of mRNA encoded by KIAA1906, corresponding to a HOST TARGET binding site such as BINDING SITE I, BINDING SITE II or BINDING SITE III. Table 2 illustrates the complementarity of the nucleotide sequences of KIAA1906 BINDING SITE, designated SEQ ID:2971, to the nucleotide sequence of VGAM356 RNA, herein designated VGAM RNA, also designated SEQ ID:691.

Another function of VGAM356 is therefore inhibition of KIAA1906 (Accession XM_{—}055095). Accordingly, utilities of VGAM356 include diagnosis, prevention and treatment of diseases and clinical conditions associated with KIAA1906. Mitogen-activated Protein Kinase 6 (MAPK6, Accession NM_{—}002748) is another VGAM356 host target gene. MAPK6 BINDING SITE is HOST TARGET binding site found in the 5′ untranslated region of mRNA encoded by MAPK6, corresponding to a HOST TARGET binding site such as BINDING SITE I, BINDING SITE II or BINDING SITE III. Table 2 illustrates the complementarity of the nucleotide sequences of MAPK6 BINDING SITE, designated SEQ ID:952, to the nucleotide sequence of VGAM356 RNA, herein designated VGAM RNA, also designated SEQ ID:691.

Another function of VGAM356 is therefore inhibition of Mitogen-activated Protein Kinase 6 (MAPK6, Accession NM_{—}002748). Accordingly, utilities of VGAM356 include diagnosis, prevention and treatment of diseases and clinical conditions associated with MAPK6. MGC13138 (Accession NM_{—}033410) is another VGAM356 host target gene. MGC13138 BINDING SITE is HOST TARGET binding site found in the 3′ untranslated region of mRNA encoded by MGC13138, corresponding to a HOST TARGET binding site such as BINDING SITE I, BINDING SITE II or BINDING SITE III. Table 2 illustrates the complementarity of the nucleotide sequences of MGC13138 BINDING SITE, designated SEQ ID:2332, to the nucleotide sequence of VGAM356 RNA, herein designated VGAM RNA, also designated SEQ ID:691.

Another function of VGAM356 is therefore inhibition of MGC13138 (Accession NM_{—}033410). Accordingly, utilities of VGAM356 include diagnosis, prevention and treatment of diseases and clinical conditions associated with MGC13138. MGC2742 (Accession NM_{—}023938) is another VGAM356 host target gene. MGC2742 BINDING SITE is HOST TARGET binding site found in the 5′ untranslated region of mRNA encoded by MGC2742, corresponding to a HOST TARGET binding site such as BINDING SITE I, BINDING SITE II or BINDING SITE III. Table 2 illustrates the complementarity of the nucleotide sequences of MGC2742 BINDING SITE, designated SEQ ID:2040, to the nucleotide sequence of VGAM356 RNA, herein designated VGAM RNA, also designated SEQ ID:691.

Another function of VGAM356 is therefore inhibition of MGC2742 (Accession NM_{—}023938). Accordingly, utilities of VGAM356 include diagnosis, prevention and treatment of diseases and clinical conditions associated with MGC2742. MGC32043 (Accession NM_{—}144582) is another VGAM356 host target gene. MGC32043 BINDING SITE is HOST TARGET binding site found in the 3′ untranslated region of mRNA encoded by MGC32043, corresponding to a HOST TARGET binding site such as BINDING SITE I, BINDING SITE II or BINDING SITE III. Table 2 illustrates the complementarity of the nucleotide sequences of MGC32043 BINDING SITE, designated SEQ ID:2486, to the nucleotide sequence of VGAM356 RNA, herein designated VGAM RNA, also designated SEQ ID:691.

Another function of VGAM356 is therefore inhibition of MGC32043 (Accession NM_{—}144582). Accordingly, utilities of VGAM356 include diagnosis, prevention and treatment of diseases and clinical conditions associated with MGC32043. Nuclear Factor of Activated T-cells 5, Tonicity-responsive (NFAT5, Accession NM_{—}138714) is another VGAM356 host target gene. NFAT5 BINDING SITE is HOST TARGET binding site found in the 3′ untranslated region of mRNA encoded by NFAT5, corresponding to a HOST TARGET binding site such as BINDING SITE I, BINDING SITE II or BINDING SITE III. Table 2 illustrates the complementarity of the nucleotide sequences of NFAT5 BINDING SITE, designated SEQ ID:2452, to the nucleotide sequence of VGAM356 RNA, herein designated VGAM RNA, also designated SEQ ID:691.

Another function of VGAM356 is therefore inhibition of Nuclear Factor of Activated T-cells 5, Tonicity-responsive (NFAT5, Accession NM_{—}138714). Accordingly, utilities of VGAM356 include diagnosis, prevention and treatment of diseases and clinical conditions associated with NFAT5. Oxysterol Binding Protein-like 8 (OSBPL8, Accession NM_{—}020841) is another VGAM356 host target gene. OSBPL8 BINDING SITE is HOST TARGET binding site found in the 3′ untranslated region of mRNA encoded by OSBPL8, corresponding to a HOST TARGET binding site such as BINDING SITE I, BINDING SITE II or BINDING SITE III. Table 2 illustrates the complementarity of the nucleotide sequences of OSBPL8 BINDING SITE, designated SEQ ID:1929, to the nucleotide sequence of VGAM356 RNA, herein designated VGAM RNA, also designated SEQ ID:691.

Another function of VGAM356 is therefore inhibition of Oxysterol Binding Protein-like 8 (OSBPL8, Accession NM_{—}020841). Accordingly, utilities of VGAM356 include diagnosis, prevention and treatment of diseases and clinical conditions associated with OSBPL8. RAB10, Member RAS Oncogene Family (RAB10, Accession XM_{—}097979) is another VGAM356 host target gene. RAB10 BINDING SITE is HOST TARGET binding site found in the 3′ untranslated region of mRNA encoded by RAB10, corresponding to a HOST TARGET binding site such as BINDING SITE I, BINDING SITE II or BINDING SITE III. Table 2 illustrates the complementarity of the nucleotide sequences of RAB10 BINDING SITE, designated SEQ ID:3337, to the nucleotide sequence of VGAM356 RNA, herein designated VGAM RNA, also designated SEQ ID:691.

Another function of VGAM356 is therefore inhibition of RAB10, Member RAS Oncogene Family (RAB10, Accession XM_{—}097979). Accordingly, utilities of VGAM356 include diagnosis, prevention and treatment of diseases and clinical conditions associated with RAB10. RAN Binding Protein 1 (RANBP1, Accession NM_{—}002882) is another VGAM356 host target gene. RANBP1 BINDING SITE is HOST TARGET binding site found in the 3′ untranslated region of mRNA encoded by RANBP1, corresponding to a HOST TARGET binding site such as BINDING SITE I, BINDING SITE II or BINDING SITE III. Table 2 illustrates the complementarity of the nucleotide sequences of RANBP1 BINDING SITE, designated SEQ ID:969, to the nucleotide sequence of VGAM356 RNA, herein designated VGAM RNA, also designated SEQ ID:691.

Another function of VGAM356 is therefore inhibition of RAN Binding Protein 1 (RANBP1, Accession NM_{—}002882). Accordingly, utilities of VGAM356 include diagnosis, prevention and treatment of diseases and clinical conditions associated with RANBP1. Ras Association (RalGDS/AF-6) Domain Family 2 (RASSF2, Accession NM_{—}014737) is another VGAM356 host target gene. RASSF2 BINDING SITE is HOST TARGET binding site found in the 3′ untranslated region of mRNA encoded by RASSF2, corresponding to a HOST TARGET binding site such as BINDING SITE I, BINDING SITE II or BINDING SITE III. Table 2 illustrates the complementarity of the nucleotide sequences of RASSF2 BINDING SITE, designated SEQ ID:1537, to the nucleotide sequence of VGAM356 RNA, herein designated VGAM RNA, also designated SEQ ID:691.

Another function of VGAM356 is therefore inhibition of Ras Association (RalGDS/AF-6) Domain Family 2 (RASSF2, Accession NM_{—}014737). Accordingly, utilities of VGAM356 include diagnosis, prevention and treatment of diseases and clinical conditions associated with RASSF2. Serine/threonine Kinase 22D (spermiogenesis associated) (STK22D, Accession NM_{—}032028) is another VGAM356 host target gene. STK22D BINDING SITE is HOST TARGET binding site found in the 3′ untranslated region of mRNA encoded by STK22D, corresponding to a HOST TARGET binding site such as BINDING SITE I, BINDING SITE II or BINDING SITE III. Table 2 illustrates the complementarity of the nucleotide sequences of STK22D BINDING SITE, designated SEQ ID:2226, to the nucleotide sequence of VGAM356 RNA, herein designated VGAM RNA, also designated SEQ ID:691.

Another function of VGAM356 is therefore inhibition of Serine/threonine Kinase 22D (spermiogenesis associated) (STK22D, Accession NM_{—}032028). Accordingly, utilities of VGAM356 include diagnosis, prevention and treatment of diseases and clinical conditions associated with STK22D. Synaptojanin 2 (SYNJ2, Accession XM_{—}029746) is another VGAM356 host target gene. SYNJ2 BINDING SITE is HOST TARGET binding site found in the 3′ untranslated region of mRNA encoded by SYNJ2, corresponding to a HOST TARGET binding site such as BINDING SITE I, BINDING SITE II or BINDING SITE III. Table 2 illustrates the complementarity of the nucleotide sequences of SYNJ2 BINDING SITE, designated SEQ ID:2606, to the nucleotide sequence of VGAM356 RNA, herein designated VGAM RNA, also designated SEQ ID:691.

Another function of VGAM356 is therefore inhibition of Synaptojanin 2 (SYNJ2, Accession XM_{—}029746). Accordingly, utilities of VGAM356 include diagnosis, prevention and treatment of diseases and clinical conditions associated with SYNJ2. TBLR1 (Accession NM_{—}024665) is another VGAM356 host target gene. TBLR1 BINDING SITE is HOST TARGET binding site found in the 3′ untranslated region of mRNA encoded by TBLR1, corresponding to a HOST TARGET binding site such as BINDING SITE I, BINDING SITE II or BINDING SITE III. Table 2 illustrates the complementarity of the nucleotide sequences of TBLR1 BINDING SITE, designated SEQ ID:2079, to the nucleotide sequence of VGAM356 RNA, herein designated VGAM RNA, also designated SEQ ID:691.

Another function of VGAM356 is therefore inhibition of TBLR1 (Accession NM_{—}024665). Accordingly, utilities of VGAM356 include diagnosis, prevention and treatment of diseases and clinical conditions associated with TBLR1. Transmembrane 9 Super family Member 1 (TM9SF1, Accession NM_{—}006405) is another VGAM356 host target gene. TM9SF1 BINDING SITE is HOST TARGET binding site found in the 3′ untranslated region of mRNA encoded by TM9SF1, corresponding to a HOST TARGET binding site such as BINDING SITE I, BINDING SITE II or BINDING SITE III. Table 2 illustrates the complementarity of the nucleotide sequences of TM9SF1 BINDING SITE, designated SEQ ID:1296, to the nucleotide sequence of VGAM356 RNA, herein designated VGAM RNA, also designated SEQ ID:691.

Another function of VGAM356 is therefore inhibition of Transmembrane 9 Super family Member 1 (TM9SF1, Accession NM_{—}006405). Accordingly, utilities of VGAM356 include diagnosis, prevention and treatment of diseases and clinical conditions associated with TM9SF1. Unc-51-like Kinase 2 (C. elegans) (ULK2, Accession NM_{—}014683) is another VGAM356 host target gene. ULK2 BINDING SITE is HOST TARGET binding site found in the 3′ untranslated region of mRNA encoded by ULK2, corresponding to a HOST TARGET binding site such as BINDING SITE I, BINDING SITE II or BINDING SITE III. Table 2 illustrates the complementarity of the nucleotide sequences of ULK2 BINDING SITE, designated SEQ ID:1523, to the nucleotide sequence of VGAM356 RNA, herein designated VGAM RNA, also designated SEQ ID:691.

Another function of VGAM356 is therefore inhibition of Unc-51-like Kinase 2 (C. elegans) (ULK2, Accession NM_{—}014683). Accordingly, utilities of VGAM356 include diagnosis, prevention and treatment of diseases and clinical conditions associated with ULK2. Unc-5 Homolog D (C. elegans) (UNC5D, Accession NM_{—}080872) is another VGAM356 host target gene. UNC5D BINDING SITE is HOST TARGET binding site found in the 3′ untranslated region of mRNA encoded by UNC5D, corresponding to a HOST TARGET binding site such as BINDING SITE I, BINDING SITE II or BINDING SITE III. Table 2 illustrates the complementarity of the nucleotide sequences of UNC5D BINDING SITE, designated SEQ ID:2393, to the nucleotide sequence of VGAM356 RNA, herein designated VGAM RNA, also designated SEQ ID:691.

Another function of VGAM356 is therefore inhibition of Unc-5 Homolog D (C. elegans) (UNC5D, Accession NM_{—}080872). Accordingly, utilities of VGAM356 include diagnosis, prevention and treatment of diseases and clinical conditions associated with UNC5D. LOC121219 (Accession XM_{—}058544) is another VGAM356 host target gene. LOC121219 BINDING SITE is HOST TARGET binding site found in the 5′ untranslated region of mRNA encoded by LOC121219, corresponding to a HOST TARGET binding site such as BINDING SITE I, BINDING SITE II or BINDING SITE III. Table 2 illustrates the complementarity of the nucleotide sequences of LOC121219 BINDING SITE, designated SEQ ID:2988, to the nucleotide sequence of VGAM356 RNA, herein designated VGAM RNA, also designated SEQ ID:691.

Another function of VGAM356 is therefore inhibition of LOC121219 (Accession XM_{—}058544). Accordingly, utilities of VGAM356 include diagnosis, prevention and treatment of diseases and clinical conditions associated with LOC121219. LOC128077 (Accession XM_{—}059208) is another VGAM356 host target gene. LOC128077 BINDING SITE is HOST TARGET binding site found in the 3′ untranslated region of mRNA encoded by LOC128077, corresponding to a HOST TARGET binding site such as BINDING SITE I, BINDING SITE II or BINDING SITE III. Table 2 illustrates the complementarity of the nucleotide sequences of LOC128077 BINDING SITE, designated SEQ ID:3006, to the nucleotide sequence of VGAM356 RNA, herein designated VGAM RNA, also designated SEQ ID:691.

Another function of VGAM356 is therefore inhibition of LOC128077 (Accession XM_{—}059208). Accordingly, utilities of VGAM356 include diagnosis, prevention and treatment of diseases and clinical conditions associated with LOC128077. LOC143451 (Accession XM_{—}084521) is another VGAM356 host target gene. LOC143451 BINDING SITE is HOST TARGET binding site found in the 3′ untranslated region of mRNA encoded by LOC143451, corresponding to a HOST TARGET binding site such as BINDING SITE I, BINDING SITE II or BINDING SITE III. Table 2 illustrates the complementarity of the nucleotide sequences of LOC143451 BINDING SITE, designated SEQ ID:3058, to the nucleotide sequence of VGAM356 RNA, herein designated VGAM RNA, also designated SEQ ID:691.

Another function of VGAM356 is therefore inhibition of LOC143451 (Accession XM_{—}084521). Accordingly, utilities of VGAM356 include diagnosis, prevention and treatment of diseases and clinical conditions associated with LOC143451. LOC145125 (Accession XM_{—}085025) is another VGAM356 host target gene. LOC145125 BINDING SITE is HOST TARGET binding site found in the 3′ untranslated region of mRNA encoded by LOC145125, corresponding to a HOST TARGET binding site such as BINDING SITE I, BINDING SITE II or BINDING SITE III. Table 2 illustrates the complementarity of the nucleotide sequences of LOC145125 BINDING SITE, designated SEQ ID:3075, to the nucleotide sequence of VGAM356 RNA, herein designated VGAM RNA, also designated SEQ ID:691.

Another function of VGAM356 is therefore inhibition of LOC145125 (Accession XM_{—}085025). Accordingly, utilities of VGAM356 include diagnosis, prevention and treatment of diseases and clinical conditions associated with LOC145125. LOC149351 (Accession XM_{—}086503) is another VGAM356 host target gene. LOC149351 BINDING SITE is HOST TARGET binding site found in the 3′ untranslated region of mRNA encoded by LOC149351, corresponding to a HOST TARGET binding site such as BINDING SITE I, BINDING SITE II or BINDING SITE III. Table 2 illustrates the complementarity of the nucleotide sequences of LOC149351 BINDING SITE, designated SEQ ID:3131, to the nucleotide sequence of VGAM356 RNA, herein designated VGAM RNA, also designated SEQ ID:691.

Another function of VGAM356 is therefore inhibition of LOC149351 (Accession XM_{—}086503). Accordingly, utilities of VGAM356 include diagnosis, prevention and treatment of diseases and clinical conditions associated with LOC149351. LOC149722 (Accession XM_{—}097709) is another VGAM356 host target gene. LOC149722 BINDING SITE is HOST TARGET binding site found in the 5′ untranslated region of mRNA encoded by LOC149722, corresponding to a HOST TARGET binding site such as BINDING SITE I, BINDING SITE II or BINDING SITE III. Table 2 illustrates the complementarity of the nucleotide sequences of LOC149722 BINDING SITE, designated SEQ ID:3312, to the nucleotide sequence of VGAM356 RNA, herein designated VGAM RNA, also designated SEQ ID:691.

Another function of VGAM356 is therefore inhibition of LOC149722 (Accession XM_{—}097709). Accordingly, utilities of VGAM356 include diagnosis, prevention and treatment of diseases and clinical conditions associated with LOC149722. LOC149837 (Accession XM_{—}097747) is another VGAM356 host target gene. LOC149837 BINDING SITE is HOST TARGET binding site found in the 5′ untranslated region of mRNA encoded by LOC149837, corresponding to a HOST TARGET binding site such as BINDING SITE I, BINDING SITE II or BINDING SITE III. Table 2 illustrates the complementarity of the nucleotide sequences of LOC149837 BINDING SITE, designated SEQ ID:3318, to the nucleotide sequence of VGAM356 RNA, herein designated VGAM RNA, also designated SEQ ID:691.

Another function of VGAM356 is therefore inhibition of LOC149837 (Accession XM_{—}097747). Accordingly, utilities of VGAM356 include diagnosis, prevention and treatment of diseases and clinical conditions associated with LOC149837. LOC150054 (Accession XM_{—}097797) is another VGAM356 host target gene. LOC150054 BINDING SITE is HOST TARGET binding site found in the 3′ untranslated region of mRNA encoded by LOC150054, corresponding to a HOST TARGET binding site such as BINDING SITE I, BINDING SITE II or BINDING SITE III. Table 2 illustrates the complementarity of the nucleotide sequences of LOC150054 BINDING SITE, designated SEQ ID:3323, to the nucleotide sequence of VGAM356 RNA, herein designated VGAM RNA, also designated SEQ ID:691.

Another function of VGAM356 is therefore inhibition of LOC150054 (Accession XM_{—}097797). Accordingly, utilities of VGAM356 include diagnosis, prevention and treatment of diseases and clinical conditions associated with LOC150054. LOC150150 (Accession XM_{—}097820) is another VGAM356 host target gene. LOC150150 BINDING SITE is HOST TARGET binding site found in the 5′ untranslated region of mRNA encoded by LOC150150, corresponding to a HOST TARGET binding site such as BINDING SITE I, BINDING SITE II or BINDING SITE III. Table 2 illustrates the complementarity of the nucleotide sequences of LOC150150 BINDING SITE, designated SEQ ID:3324, to the nucleotide sequence of VGAM356 RNA, herein designated VGAM RNA, also designated SEQ ID:691.

Another function of VGAM356 is therefore inhibition of LOC150150 (Accession XM_{—}097820). Accordingly, utilities of VGAM356 include diagnosis, prevention and treatment of diseases and clinical conditions associated with LOC150150. LOC150225 (Accession XM_{—}097870) is another VGAM356 host target gene. LOC150225 BINDING SITE is HOST TARGET binding site found in the 3′ untranslated region of mRNA encoded by LOC150225, corresponding to a HOST TARGET binding site such as BINDING SITE I, BINDING SITE II or BINDING SITE III. Table 2 illustrates the complementarity of the nucleotide sequences of LOC150225 BINDING SITE, designated SEQ ID:3327, to the nucleotide sequence of VGAM356 RNA, herein designated VGAM RNA, also designated SEQ ID:691.

Another function of VGAM356 is therefore inhibition of LOC150225 (Accession XM_{—}097870). Accordingly, utilities of VGAM356 include diagnosis, prevention and treatment of diseases and clinical conditions associated with LOC150225. LOC152816 (Accession XM_{—}098270) is another VGAM356 host target gene. LOC152816 BINDING SITE is HOST TARGET binding site found in the 3′ untranslated region of mRNA encoded by LOC152816, corresponding to a HOST TARGET binding site such as BINDING SITE I, BINDING SITE II or BINDING SITE III. Table 2 illustrates the complementarity of the nucleotide sequences of LOC152816 BINDING SITE, designated SEQ ID:3364, to the nucleotide sequence of VGAM356 RNA, herein designated VGAM RNA, also designated SEQ ID:691.

Another function of VGAM356 is therefore inhibition of LOC152816 (Accession XM_{—}098270). Accordingly, utilities of VGAM356 include diagnosis, prevention and treatment of diseases and clinical conditions associated with LOC152816. LOC155038 (Accession XM_{—}088130) is another VGAM356 host target gene. LOC155038 BINDING SITE is HOST TARGET binding site found in the 3′ untranslated region of mRNA encoded by LOC155038, corresponding to a HOST TARGET binding site such as BINDING SITE I, BINDING SITE II or BINDING SITE III. Table 2 illustrates the complementarity of the nucleotide sequences of LOC155038 BINDING SITE, designated SEQ ID:3198, to the nucleotide sequence of VGAM356 RNA, herein designated VGAM RNA, also designated SEQ ID:691.

Another function of VGAM356 is therefore inhibition of LOC155038 (Accession XM_{—}088130). Accordingly, utilities of VGAM356 include diagnosis, prevention and treatment of diseases and clinical conditions associated with LOC155038. LOC158629 (Accession XM_{—}098972) is another VGAM356 host target gene. LOC158629 BINDING SITE is HOST TARGET binding site found in the 3′ untranslated region of mRNA encoded by LOC158629, corresponding to a HOST TARGET binding site such as BINDING SITE I, BINDING SITE II or BINDING SITE III. Table 2 illustrates the complementarity of the nucleotide sequences of LOC158629 BINDING SITE, designated SEQ ID:3394, to the nucleotide sequence of VGAM356 RNA, herein designated VGAM RNA, also designated SEQ ID:691.

Another function of VGAM356 is therefore inhibition of LOC158629 (Accession XM_{—}098972). Accordingly, utilities of VGAM356 include diagnosis, prevention and treatment of diseases and clinical conditions associated with LOC158629. LOC158943 (Accession XM_{—}018400) is another VGAM356 host target gene. LOC158943 BINDING SITE is HOST TARGET binding site found in the 3′ untranslated region of mRNA encoded by LOC158943, corresponding to a HOST TARGET binding site such as BINDING SITE I, BINDING SITE II or BINDING SITE III. Table 2 illustrates the complementarity of the nucleotide sequences of LOC158943 BINDING SITE, designated SEQ ID:2572, to the nucleotide sequence of VGAM356 RNA, herein designated VGAM RNA, also designated SEQ ID:691.

Another function of VGAM356 is therefore inhibition of LOC158943 (Accession XM_{—}018400). Accordingly, utilities of VGAM356 include diagnosis, prevention and treatment of diseases and clinical conditions associated with LOC158943. LOC170082 (Accession XM_{—}093092) is another VGAM356 host target gene. LOC170082 BINDING SITE is HOST TARGET binding site found in the 3′ untranslated region of mRNA encoded by LOC170082, corresponding to a HOST TARGET binding site such as BINDING SITE I, BINDING SITE II or BINDING SITE III. Table 2 illustrates the complementarity of the nucleotide sequences of LOC170082 BINDING SITE, designated SEQ ID:3254, to the nucleotide sequence of VGAM356 RNA, herein designated VGAM RNA, also designated SEQ ID:691.

Another function of VGAM356 is therefore inhibition of LOC170082 (Accession XM_{—}093092). Accordingly, utilities of VGAM356 include diagnosis, prevention and treatment of diseases and clinical conditions associated with LOC170082. LOC199920 (Accession XM_{—}114056) is another VGAM356 host target gene. LOC199920 BINDING SITE is HOST TARGET binding site found in the 5′ untranslated region of mRNA encoded by LOC199920, corresponding to a HOST TARGET binding site such as BINDING SITE I, BINDING SITE II or BINDING SITE III. Table 2 illustrates the complementarity of the nucleotide sequences of LOC199920 BINDING SITE, designated SEQ ID:3436, to the nucleotide sequence of VGAM356 RNA, herein designated VGAM RNA, also designated SEQ ID:691.

Another function of VGAM356 is therefore inhibition of LOC199920 (Accession XM_{—}114056). Accordingly, utilities of VGAM356 include diagnosis, prevention and treatment of diseases and clinical conditions associated with LOC199920. LOC219667 (Accession XM_{—}166098) is another VGAM356 host target gene. LOC219667 BINDING SITE is HOST TARGET binding site found in the 3′ untranslated region of mRNA encoded by LOC219667, corresponding to a HOST TARGET binding site such as BINDING SITE I, BINDING SITE II or BINDING SITE III. Table 2 illustrates the complementarity of the nucleotide sequences of LOC219667 BINDING SITE, designated SEQ ID:3516, to the nucleotide sequence of VGAM356 RNA, herein designated VGAM RNA, also designated SEQ ID:691.

Another function of VGAM356 is therefore inhibition of LOC219667 (Accession XM_{—}166098). Accordingly, utilities of VGAM356 include diagnosis, prevention and treatment of diseases and clinical conditions associated with LOC219667. LOC220279 (Accession XM_{—}169083) is another VGAM356 host target gene. LOC220279 BINDING SITE is HOST TARGET binding site found in the 5′ untranslated region of mRNA encoded by LOC220279, corresponding to a HOST TARGET binding site such as BINDING SITE I, BINDING SITE II or BINDING SITE III. Table 2 illustrates the complementarity of the nucleotide sequences of LOC220279 BINDING SITE, designated SEQ ID:3653, to the nucleotide sequence of VGAM356 RNA, herein designated VGAM RNA, also designated SEQ ID:691.

Another function of VGAM356 is therefore inhibition of LOC220279 (Accession XM_{—}169083). Accordingly, utilities of VGAM356 include diagnosis, prevention and treatment of diseases and clinical conditions associated with LOC220279. LOC221830 (Accession XM_{—}166508) is another VGAM356 host target gene. LOC221830 BINDING SITE is HOST TARGET binding site found in the 3′ untranslated region of mRNA encoded by LOC221830, corresponding to a HOST TARGET binding site such as BINDING SITE I, BINDING SITE II or BINDING SITE III. Table 2 illustrates the complementarity of the nucleotide sequences of LOC221830 BINDING SITE, designated SEQ ID:3567, to the nucleotide sequence of VGAM356 RNA, herein designated VGAM RNA, also designated SEQ ID:691.

Another function of VGAM356 is therefore inhibition of LOC221830 (Accession XM_{—}166508). Accordingly, utilities of VGAM356 include diagnosis, prevention and treatment of diseases and clinical conditions associated with LOC221830. LOC222166 (Accession XM_{—}168425) is another VGAM356 host target gene. LOC222166 BINDING SITE is HOST TARGET binding site found in the 3′ untranslated region of mRNA encoded by LOC222166, corresponding to a HOST TARGET binding site such as BINDING SITE I, BINDING SITE II or BINDING SITE III. Table 2 illustrates the complementarity of the nucleotide sequences of LOC222166 BINDING SITE, designated SEQ ID:3635, to the nucleotide sequence of VGAM356 RNA, herein designated VGAM RNA, also designated SEQ ID:691.

Another function of VGAM356 is therefore inhibition of LOC222166 (Accession XM_{—}168425). Accordingly, utilities of VGAM356 include diagnosis, prevention and treatment of diseases and clinical conditions associated with LOC222166. LOC254440 (Accession XM_{—}173126) is another VGAM356 host target gene. LOC254440 BINDING SITE is HOST TARGET binding site found in the 3′ untranslated region of mRNA encoded by LOC254440, corresponding to a HOST TARGET binding site such as BINDING SITE I, BINDING SITE II or BINDING SITE III. Table 2 illustrates the complementarity of the nucleotide sequences of LOC254440 BINDING SITE, designated SEQ ID:3729, to the nucleotide sequence of VGAM356 RNA, herein designated VGAM RNA, also designated SEQ ID:691.

Another function of VGAM356 is therefore inhibition of LOC254440 (Accession XM_{—}173126). Accordingly, utilities of VGAM356 include diagnosis, prevention and treatment of diseases and clinical conditions associated with LOC254440. LOC51123 (Accession XM_{—}018277) is another VGAM356 host target gene. LOC51123 BINDING SITE is HOST TARGET binding site found in the 3′ untranslated region of mRNA encoded by LOC51123, corresponding to a HOST TARGET binding site such as BINDING SITE I, BINDING SITE II or BINDING SITE III. Table 2 illustrates the complementarity of the nucleotide sequences of LOC51123 BINDING SITE, designated SEQ ID:2571, to the nucleotide sequence of VGAM356 RNA, herein designated VGAM RNA, also designated SEQ ID:691.

Another function of VGAM356 is therefore inhibition of LOC51123 (Accession XM_{—}018277). Accordingly, utilities of VGAM356 include diagnosis, prevention and treatment of diseases and clinical conditions associated with LOC51123. LOC51596 (Accession NM_{—}015921) is another VGAM356 host target gene. LOC51596 BINDING SITE is HOST TARGET binding site found in the 5′ untranslated region of mRNA encoded by LOC51596, corresponding to a HOST TARGET binding site such as BINDING SITE I, BINDING SITE II or BINDING SITE III. Table 2 illustrates the complementarity of the nucleotide sequences of LOC51596 BINDING SITE, designated SEQ ID:1651, to the nucleotide sequence of VGAM356 RNA, herein designated VGAM RNA, also designated SEQ ID:691.

Another function of VGAM356 is therefore inhibition of LOC51596 (Accession NM_{—}015921). Accordingly, utilities of VGAM356 include diagnosis, prevention and treatment of diseases and clinical conditions associated with LOC51596. LOC84570 (Accession NM_{—}032518) is another VGAM356 host target gene. LOC84570 BINDING SITE is HOST TARGET binding site found in the 5′ untranslated region of mRNA encoded by LOC84570, corresponding to a HOST TARGET binding site such as BINDING SITE I, BINDING SITE II or BINDING SITE III. Table 2 illustrates the complementarity of the nucleotide sequences of LOC84570 BINDING SITE, designated SEQ ID:2257, to the nucleotide sequence of VGAM356 RNA, herein designated VGAM RNA, also designated SEQ ID:691.

Another function of VGAM356 is therefore inhibition of LOC84570 (Accession NM_{—}032518). Accordingly, utilities of VGAM356 include diagnosis, prevention and treatment of diseases and clinical conditions associated with LOC84570. LOC92710 (Accession XM_{—}046811) is another VGAM356 host target gene. LOC92710 BINDING SITE is HOST TARGET binding site found in the 5′ untranslated region of mRNA encoded by LOC92710, corresponding to a HOST TARGET binding site such as BINDING SITE I, BINDING SITE II or BINDING SITE III. Table 2 illustrates the complementarity of the nucleotide sequences of LOC92710 BINDING SITE, designated SEQ ID:2885, to the nucleotide sequence of VGAM356 RNA, herein designated VGAM RNA, also designated SEQ ID:691.

Another function of VGAM356 is therefore inhibition of LOC92710 (Accession XM_{—}046811). Accordingly, utilities of VGAM356 include diagnosis, prevention and treatment of diseases and clinical conditions associated with LOC92710. FIG. 1 further provides a conceptual description of a novel bioinformatically detected viral gene of the present invention, referred to here as Viral Genomic Address Messenger 357 (VGAM357) viral gene, which modulates expression of respective host target genes thereof, the function and utility of which host target genes is known in the art.

VGAM357 is a novel bioinformatically detected regulatory, non protein coding, viral micro RNA (miRNA) gene. The method by which VGAM357 was detected is described hereinabove with reference to FIGS. 1-8.

VGAM357 gene, herein designated VGAM GENE, is a viral gene contained in the genome of Vaccinia Virus. VGAM357 host target gene, herein designated VGAM HOST TARGET GENE, is a human gene contained in the human genome.

VGAM357 gene encodes a VGAM357 precursor RNA, herein designated VGAM PRECURSOR RNA. Similar to other miRNA genes, and unlike most ordinary genes, VGAM357 precursor RNA does not encode a protein. A nucleotide sequence identical or highly similar to the nucleotide sequence of VGAM357 precursor RNA is designated SEQ ID:343, and is provided hereinbelow with reference to the sequence listing part. Nucleotide sequence SEQ ID:343 is located at position 2870 relative to the genome of Vaccinia Virus.

VGAM357 precursor RNA folds onto itself, forming VGAM357 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM357 folded precursor RNA into VGAM357 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 81%) nucleotide sequence of VGAM357 RNA is designated SEQ ID:692, and is provided hereinbelow with reference to the sequence listing part.

VGAM357 host target gene, herein designated VGAM HOST TARGET GENE, encodes a corresponding messenger RNA, VGAM357 host target RNA, herein designated VGAM HOST TARGET RNA. VGAM357 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM357 RNA, herein designated VGAM RNA, binds complementarily to one or more host target binding sites located in untranslated regions of VGAM357 host target RNA, herein designated VGAM HOST TARGET RNA. This complementary binding is due to the fact that the nucleotide sequence of VGAM357 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM357 RNA, herein designated VGAM RNA, may have a different number of host target binding sites in untranslated regions of a VGAM357 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM357 RNA, herein designated VGAM RNA, to host target binding sites on VGAM357 host target RNA, herein designated VGAM HOST TARGET RNA, such as BINDING SITE I, BINDING SITE II and BINDING SITE III, inhibits translation of VGAM357 host target RNA into VGAM357 host target protein, herein designated VGAM HOST TARGET PROTEIN. VGAM host target protein is therefore outlined by a broken line.

It is appreciated that VGAM357 host target gene, herein designated VGAM HOST TARGET GENE, in fact represents a plurality of VGAM357 host target genes. The mRNA of each one of this plurality of VGAM357 host target genes comprises one or more host target binding sites, each having a nucleotide sequence which is at least partly complementary to VGAM357 RNA, herein designated VGAM RNA, and which when bound by VGAM357 RNA causes inhibition of translation of respective one or more VGAM357 host target proteins.

It is further appreciated by one skilled in the art that the mode of translational inhibition illustrated by FIG. 1 with specific reference to translational inhibition exerted by VGAM357 gene, herein designated VGAM GENE, on one or more VGAM357 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM357 is inhibition of expression of host target genes, as part of a novel viral mechanism of attacking a host. Accordingly, utilities of VGAM357 include diagnosis, prevention and treatment of viral infection by Vaccinia Virus. Specific functions, and accordingly utilities, of VGAM357 correlate with, and may be deduced from, the identity of the host target genes which VGAM357 binds and inhibits, and the function of these host target genes, as elaborated hereinbelow.

Nucleotide sequences of the VGAM357 precursor RNA, herein designated VGAM PRECURSOR RNA, and of the ‘diced’ VGAM357 RNA, herein designated VGAM RNA, and a schematic representation of the secondary folding of VGAM357 folded precursor RNA, herein designated VGAM FOLDED PRECURSOR RNA, of VGAM357 are further described hereinbelow with reference to Table 1.

Nucleotide sequences of host target binding sites, such as BINDING SITE-I, BINDING SITE-II and BINDING SITE-III of FIG. 1, found on VGAM357 host target RNA, and schematic representation of the complementarity of each of these host target binding sites to VGAM357 RNA, herein designated VGAM RNA, are described hereinbelow with reference to Table 2.

As mentioned hereinabove with reference to FIG. 1, a function of VGAM357 gene, herein designated VGAM is inhibition of expression of VGAM357 target genes. It is appreciated that specific functions, and accordingly utilities, of VGAM357 correlate with, and may be deduced from, the identity of the target genes which VGAM357 binds and inhibits, and the function of these target genes, as elaborated hereinbelow.

Fanconi Anemia, Complementation Group F (FANCF, Accession NM_{—}022725) is a VGAM357 host target gene. FANCF BINDING SITE is HOST TARGET binding site found in the 3′ untranslated region of mRNA encoded by FANCF, corresponding to a HOST TARGET binding site such as BINDING SITE I, BINDING SITE II or BINDING SITE III. Table 2 illustrates the complementarity of the nucleotide sequences of FANCF BINDING SITE, designated SEQ ID:1997, to the nucleotide sequence of VGAM357 RNA, herein designated VGAM RNA, also designated SEQ ID:692.

A function of VGAM357 is therefore inhibition of Fanconi Anemia, Complementation Group F (FANCF, Accession NM_{—}022725). Accordingly, utilities of VGAM357 include diagnosis, prevention and treatment of diseases and clinical conditions associated with FANCF. G Protein-coupled Receptor 65 (GPR65, Accession XM_{—}007392) is another VGAM357 host target gene. GPR65 BINDING SITE is HOST TARGET binding site found in the 5′ untranslated region of mRNA encoded by GPR65, corresponding to a HOST TARGET binding site such as BINDING SITE I, BINDING SITE II or BINDING SITE III. Table 2 illustrates the complementarity of the nucleotide sequences of GPR65 BINDING SITE, designated SEQ ID:2544, to the nucleotide sequence of VGAM357 RNA, herein designated VGAM RNA, also designated SEQ ID:692.

Another function of VGAM357 is therefore inhibition of G Protein-coupled Receptor 65 (GPR65, Accession XM_{—}007392). Accordingly, utilities of VGAM357 include diagnosis, prevention and treatment of diseases and clinical conditions associated with GPR65. Lamin B1 (LMNB1, Accession NM_{—}005573) is another VGAM357 host target gene. LMNB1 BINDING SITE is HOST TARGET binding site found in the 3′ untranslated region of mRNA encoded by LMNB1, corresponding to a HOST TARGET binding site such as BINDING SITE I, BINDING SITE II or BINDING SITE III. Table 2 illustrates the complementarity of the nucleotide sequences of LMNB1 BINDING SITE, designated SEQ ID:1226, to the nucleotide sequence of VGAM357 RNA, herein designated VGAM RNA, also designated SEQ ID:692.

Another function of VGAM357 is therefore inhibition of Lamin B1 (LMNB1, Accession NM_{—}005573), a gene which is thought to provide a framework for the nuclear envelope and may also interact with chromatin. Accordingly, utilities of VGAM357 include diagnosis, prevention and treatment of diseases and clinical conditions associated with LMNB1. The function of LMNB1 and its association with various diseases and clinical conditions, has been established by previous studies, as described hereinabove with reference to VGAM50. Multiple Endocrine Neoplasia I (MEN1, Accession XM_{—}167804) is another VGAM357 host target gene. MEN1 BINDING SITE is HOST TARGET binding site found in the 5′ untranslated region of mRNA encoded by MEN1, corresponding to a HOST TARGET binding site such as BINDING SITE I, BINDING SITE II or BINDING SITE III. Table 2 illustrates the complementarity of the nucleotide sequences of MEN1 BINDING SITE, designated SEQ ID:3600, to the nucleotide sequence of VGAM357 RNA, herein designated VGAM RNA, also designated SEQ ID:692.

Another function of VGAM357 is therefore inhibition of Multiple Endocrine Neoplasia I (MEN1, Accession XM_{—}167804). Accordingly, utilities of VGAM357 include diagnosis, prevention and treatment of diseases and clinical conditions associated with MEN1. Pleiomorphic Adenoma Gene 1 (PLAG1, Accession NM_{—}002655) is another VGAM357 host target gene. PLAG1 BINDING SITE is HOST TARGET binding site found in the 3′ untranslated region of mRNA encoded by PLAG1, corresponding to a HOST TARGET binding site such as BINDING SITE I, BINDING SITE II or BINDING SITE III. Table 2 illustrates the complementarity of the nucleotide sequences of PLAG1 BINDING SITE, designated SEQ ID:944, to the nucleotide sequence of VGAM357 RNA, herein designated VGAM RNA, also designated SEQ ID:692.

Another function of VGAM357 is therefore inhibition of Pleiomorphic Adenoma Gene 1 (PLAG1, Accession NM_{—}002655), a gene which contains a zinc finger domain. Accordingly, utilities of VGAM357 include diagnosis, prevention and treatment of diseases and clinical conditions associated with PLAG1. The function of PLAG1 and its association with various diseases and clinical conditions, has been established by previous studies, as described hereinabove with reference to VGAM50. S100 Calcium Binding Protein All (calgizzarin) (S100A11, Accession NM_{—}005620) is another VGAM357 host target gene. S100A11 BINDING SITE is HOST TARGET binding site found in the 3′ untranslated region of mRNA encoded by S100A11, corresponding to a HOST TARGET binding site such as BINDING SITE I, BINDING SITE II or BINDING SITE III. Table 2 illustrates the complementarity of the nucleotide sequences of S100A11 BINDING SITE, designated SEQ ID:1231, to the nucleotide sequence of VGAM357 RNA, herein designated VGAM RNA, also designated SEQ ID:692.

Another function of VGAM357 is therefore inhibition of S100 Calcium Binding Protein All (calgizzarin) (S100A11, Accession NM_{—}005620), a gene which is a tissue/cell type specific calcium-binding protein that interacts with target proteins to link extracellular stimuli to cellular responses; member of the S100 family. Accordingly, utilities of VGAM357 include diagnosis, prevention and treatment of diseases and clinical conditions associated with S100A11. The function of S100A11 and its association with various diseases and clinical conditions, has been established by previous studies, as described hereinabove with reference to VGAM50. Solute Carrier Family 9 (sodium/hydrogen exchanger), Isoform 6 (SLC9A6, Accession NM_{—}006359) is another VGAM357 host target gene. SLC9A6 BINDING SITE is HOST TARGET binding site found in the 3′ untranslated region of mRNA encoded by SLC9A6, corresponding to a HOST TARGET binding site such as BINDING SITE I, BINDING SITE II or BINDING SITE III. Table 2 illustrates the complementarity of the nucleotide sequences of SLC9A6 BINDING SITE, designated SEQ ID:1290, to the nucleotide sequence of VGAM357 RNA, herein designated VGAM RNA, also designated SEQ ID:692.

Another function of VGAM357 is therefore inhibition of Solute Carrier Family 9 (sodium/hydrogen exchanger), Isoform 6 (SLC9A6, Accession NM_{—}006359), a gene which is involved electroneutral exchange of protons for na+ and k+ across the mitochondrial inner membrane. Accordingly, utilities of VGAM357 include diagnosis, prevention and treatment of diseases and clinical conditions associated with SLC9A6. The function of SLC9A6 and its association with various diseases and clinical conditions, has been established by previous studies, as described hereinabove with reference to VGAM50. Superoxide Dismutase 2, Mitochondrial (SOD2, Accession NM_{—}000636) is another VGAM357 host target gene. SOD2 BINDING SITE is HOST TARGET binding site found in the 3′ untranslated region of mRNA encoded by SOD2, corresponding to a HOST TARGET binding site such as BINDING SITE I, BINDING SITE II or BINDING SITE III. Table 2 illustrates the complementarity of the nucleotide sequences of SOD2 BINDING SITE, designated SEQ ID:766, to the nucleotide sequence of VGAM357 RNA, herein designated VGAM RNA, also designated SEQ ID:692.

Another function of VGAM357 is therefore inhibition of Superoxide Dismutase 2, Mitochondrial (SOD2, Accession NM_{—}000636), a gene which is intramitochondrial free radical scavenging enzyme. When he has polymorphism in signal peptides he could cause diseases of distribution. Accordingly, utilities of VGAM357 include diagnosis, prevention and treatment of diseases and clinical conditions associated with SOD2. The function of SOD2 and its association with various diseases and clinical conditions, has been established by previous studies, as described hereinabove with reference to VGAM50. Baculoviral IAP Repeat-containing 2 (BIRC2, Accession XM_{—}040717) is another VGAM357 host target gene. BIRC2 BINDING SITE is HOST TARGET binding site found in the 5′ untranslated region of mRNA encoded by BIRC2, corresponding to a HOST TARGET binding site such as BINDING SITE I, BINDING SITE II or BINDING SITE III. Table 2 illustrates the complementarity of the nucleotide sequences of BIRC2 BINDING SITE, designated SEQ ID:2784, to the nucleotide sequence of VGAM357 RNA, herein designated VGAM RNA, also designated SEQ ID:692.

Another function of VGAM357 is therefore inhibition of Baculoviral IAP Repeat-containing 2 (BIRC2, Accession XM_{—}040717). Accordingly, utilities of VGAM357 include diagnosis, prevention and treatment of diseases and clinical conditions associated with BIRC2. Chromosome X Open Reading Frame 1 (CXorf1, Accession NM_{—}004709) is another VGAM357 host target gene. CXorf1 BINDING SITE is HOST TARGET binding site found in the 5′ untranslated region of mRNA encoded by CXorf1, corresponding to a HOST TARGET binding site such as BINDING SITE I, BINDING SITE II or BINDING SITE III. Table 2 illustrates the complementarity of the nucleotide sequences of CXorf1 BINDING SITE, designated SEQ ID:1145, to the nucleotide sequence of VGAM357 RNA, herein designated VGAM RNA, also designated SEQ ID:692.

Another function of VGAM357 is therefore inhibition of Chromosome X Open Reading Frame 1 (CXorf1, Accession NM_{—}004709). Accordingly, utilities of VGAM357 include diagnosis, prevention and treatment of diseases and clinical conditions associated with CXorf1. KIAA0367 (Accession XM_{—}041018) is another VGAM357 host target gene. KIAA0367 BINDING SITE is HOST TARGET binding site found in the 3′ untranslated region of mRNA encoded by KIAA0367, corresponding to a HOST TARGET binding site such as BINDING SITE I, BINDING SITE II or BINDING SITE III. Table 2 illustrates the complementarity of the nucleotide sequences of KIAA0367 BINDING SITE, designated SEQ ID:2787, to the nucleotide sequence of VGAM357 RNA, herein designated VGAM RNA, also designated SEQ ID:692.

Another function of VGAM357 is therefore inhibition of KIAA0367 (Accession XM_{—}041018). Accordingly, utilities of VGAM357 include diagnosis, prevention and treatment of diseases and clinical conditions associated with KIAA0367. KIAA1321 (Accession XM_{—}030856) is another VGAM357 host target gene. KIAA1321 BINDING SITE is HOST TARGET binding site found in the 3′ untranslated region of mRNA encoded by KIAA1321, corresponding to a HOST TARGET binding site such as BINDING SITE I, BINDING SITE II or BINDING SITE III. Table 2 illustrates the complementarity of the nucleotide sequences of KIAA1321 BINDING SITE, designated SEQ ID:2627, to the nucleotide sequence of VGAM357 RNA, herein designated VGAM RNA, also designated SEQ ID:692.

Another function of VGAM357 is therefore inhibition of KIAA1321 (Accession XM_{—}030856). Accordingly, utilities of VGAM357 include diagnosis, prevention and treatment of diseases and clinical conditions associated with KIAA1321. KIAA1718 (Accession XM_{—}034823) is another VGAM357 host target gene. KIAA1718 BINDING SITE is HOST TARGET binding site found in the 3′ untranslated region of mRNA encoded by KIAA1718, corresponding to a HOST TARGET binding site such as BINDING SITE I, BINDING SITE II or BINDING SITE III. Table 2 illustrates the complementarity of the nucleotide sequences of KIAA1718 BINDING SITE, designated SEQ ID:2686, to the nucleotide sequence of VGAM357 RNA, herein designated VGAM RNA, also designated SEQ ID:692.

Another function of VGAM357 is therefore inhibition of KIAA1718 (Accession XM_{—}034823). Accordingly, utilities of VGAM357 include diagnosis, prevention and treatment of diseases and clinical conditions associated with KIAA1718. KIAA1789 (Accession XM_{—}040486) is another VGAM357 host target gene. KIAA1789 BINDING SITE is HOST TARGET binding site found in the 5′ untranslated region of mRNA encoded by KIAA1789, corresponding to a HOST TARGET binding site such as BINDING SITE I, BINDING SITE II or BINDING SITE III. Table 2 illustrates the complementarity of the nucleotide sequences of KIAA1789 BINDING SITE, designated SEQ ID:2781, to the nucleotide sequence of VGAM357 RNA, herein designated VGAM RNA, also designated SEQ ID:692.

Another function of VGAM357 is therefore inhibition of KIAA1789 (Accession XM_{—}040486). Accordingly, utilities of VGAM357 include diagnosis, prevention and treatment of diseases and clinical conditions associated with KIAA1789. PRO2964 (Accession NM_{—}018547) is another VGAM357 host target gene. PRO2964 BINDING SITE is HOST TARGET binding site found in the 3′ untranslated region of mRNA encoded by PRO2964, corresponding to a HOST TARGET binding site such as BINDING SITE I, BINDING SITE II or BINDING SITE III. Table 2 illustrates the complementarity of the nucleotide sequences of PRO2964 BINDING SITE, designated SEQ ID:1842, to the nucleotide sequence of VGAM357 RNA, herein designated VGAM RNA, also designated SEQ ID:692.

Another function of VGAM357 is therefore inhibition of PRO2964 (Accession NM_{—}018547). Accordingly, utilities of VGAM357 include diagnosis, prevention and treatment of diseases and clinical conditions associated with PRO2964. Solute Carrier Family 17 (sodium-dependent inorganic phosphate cotransporter), Member 6 (SLC17A6, Accession NM_{—}020346) is another VGAM357 host target gene. SLC17A6 BINDING SITE is HOST TARGET binding site found in the 3′ untranslated region of mRNA encoded by SLC17A6, corresponding to a HOST TARGET binding site such as BINDING SITE I, BINDING SITE II or BINDING SITE III. Table 2 illustrates the complementarity of the nucleotide sequences of SLC17A6 BINDING SITE, designated SEQ ID:1912, to the nucleotide sequence of VGAM357 RNA, herein designated VGAM RNA, also designated SEQ ID:692.

Another function of VGAM357 is therefore inhibition of Solute Carrier Family 17 (sodium-dependent inorganic phosphate cotransporter), Member 6 (SLC17A6, Accession NM_{—}020346). Accordingly, utilities of VGAM357 include diagnosis, prevention and treatment of diseases and clinical conditions associated with SLC17A6. TNF Receptor-associated Factor 3 (TRAF3, Accession XM_{—}007256) is another VGAM357 host target gene. TRAF3 BINDING SITE is HOST TARGET binding site found in the 5′ untranslated region of mRNA encoded by TRAF3, corresponding to a HOST TARGET binding site such as BINDING SITE I, BINDING SITE II or BINDING SITE III. Table 2 illustrates the complementarity of the nucleotide sequences of TRAF3 BINDING SITE, designated SEQ ID:2542, to the nucleotide sequence of VGAM357 RNA, herein designated VGAM RNA, also designated SEQ ID:692.

Another function of VGAM357 is therefore inhibition of TNF Receptor-associated Factor 3 (TRAF3, Accession XM_{—}007256). Accordingly, utilities of VGAM357 include diagnosis, prevention and treatment of diseases and clinical conditions associated with TRAF3. LOC151361 (Accession XM_{—}098048) is another VGAM357 host target gene. LOC151361 BINDING SITE is HOST TARGET binding site found in the 3′ untranslated region of mRNA encoded by LOC151361, corresponding to a HOST TARGET binding site such as BINDING SITE I, BINDING SITE II or BINDING SITE III. Table 2 illustrates the complementarity of the nucleotide sequences of LOC151361 BINDING SITE, designated SEQ ID:3344, to the nucleotide sequence of VGAM357 RNA, herein designated VGAM RNA, also designated SEQ ID:692.

Another function of VGAM357 is therefore inhibition of LOC151361 (Accession XM_{—}098048). Accordingly, utilities of VGAM357 include diagnosis, prevention and treatment of diseases and clinical conditions associated with LOC151361. LOC158714 (Accession XM_{—}088650) is another VGAM357 host target gene. LOC158714 BINDING SITE is HOST TARGET binding site found in the 3′ untranslated region of mRNA encoded by LOC158714, corresponding to a HOST TARGET binding site such as BINDING SITE I, BINDING SITE II or BINDING SITE III. Table 2 illustrates the complementarity of the nucleotide sequences of LOC158714 BINDING SITE, designated SEQ ID:3224, to the nucleotide sequence of VGAM357 RNA, herein designated VGAM RNA, also designated SEQ ID:692.

Another function of VGAM357 is therefore inhibition of LOC158714 (Accession XM_{—}088650). Accordingly, utilities of VGAM357 include diagnosis, prevention and treatment of diseases and clinical conditions associated with LOC158714. LOC219667 (Accession XM_{—}166098) is another VGAM357 host target gene. LOC219667 BINDING SITE is HOST TARGET binding site found in the 3′ untranslated region of mRNA encoded by LOC219667, corresponding to a HOST TARGET binding site such as BINDING SITE I, BINDING SITE II or BINDING SITE III. Table 2 illustrates the complementarity of the nucleotide sequences of LOC219667 BINDING SITE, designated SEQ ID:3518, to the nucleotide sequence of VGAM357 RNA, herein designated VGAM RNA, also designated SEQ ID:692.

Another function of VGAM357 is therefore inhibition of LOC219667 (Accession XM_{—}166098). Accordingly, utilities of VGAM357 include diagnosis, prevention and treatment of diseases and clinical conditions associated with LOC219667. LOC91565 (Accession XM_{—}039231) is another VGAM357 host target gene. LOC91565 BINDING SITE is HOST TARGET binding site found in the 3′ untranslated region of mRNA encoded by LOC91565, corresponding to a HOST TARGET binding site such as BINDING SITE I, BINDING SITE II or BINDING SITE III. Table 2 illustrates the complementarity of the nucleotide sequences of LOC91565 BINDING SITE, designated SEQ ID:2749, to the nucleotide sequence of VGAM357 RNA, herein designated VGAM RNA, also designated SEQ ID:692.

Another function of VGAM357 is therefore inhibition of LOC91565 (Accession XM_{—}039231). Accordingly, utilities of VGAM357 include diagnosis, prevention and treatment of diseases and clinical conditions associated with LOC91565. FIG. 1 further provides a conceptual description of a novel bioinformatically detected viral gene of the present invention, referred to here as Viral Genomic Address Messenger 358 (VGAM358) viral gene, which modulates expression of respective host target genes thereof, the function and utility of which host target genes is known in the art.

VGAM358 is a novel bioinformatically detected regulatory, non protein coding, viral micro RNA (miRNA) gene. The method by which VGAM358 was detected is described hereinabove with reference to FIGS. 1-8.

VGAM358 gene, herein designated VGAM GENE, is a viral gene contained in the genome of Vaccinia Virus. VGAM358 host target gene, herein designated VGAM HOST TARGET GENE, is a human gene contained in the human genome.

VGAM358 gene encodes a VGAM358 precursor RNA, herein designated VGAM PRECURSOR RNA. Similar to other miRNA genes, and unlike most ordinary genes, VGAM358 precursor RNA does not encode a protein. A nucleotide sequence identical or highly similar to the nucleotide sequence of VGAM358 precursor RNA is designated SEQ ID:344, and is provided hereinbelow with reference to the sequence listing part. Nucleotide sequence SEQ ID:344 is located at position 151547 relative to the genome of Vaccinia Virus.

VGAM358 precursor RNA folds onto itself, forming VGAM358 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM358 folded precursor RNA into VGAM358 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 92%) nucleotide sequence of VGAM358 RNA is designated SEQ ID:693, and is provided hereinbelow with reference to the sequence listing part.

VGAM358 host target gene, herein designated VGAM HOST TARGET GENE, encodes a corresponding messenger RNA, VGAM358 host target RNA, herein designated VGAM HOST TARGET RNA. VGAM358 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM358 RNA, herein designated VGAM RNA, binds complementarily to one or more host target binding sites located in untranslated regions of VGAM358 host target RNA, herein designated VGAM HOST TARGET RNA. This complementary binding is due to the fact that the nucleotide sequence of VGAM358 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM358 RNA, herein designated VGAM RNA, may have a different number of host target binding sites in untranslated regions of a VGAM358 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM358 RNA, herein designated VGAM RNA, to host target binding sites on VGAM358 host target RNA, herein designated VGAM HOST TARGET RNA, such as BINDING SITE I, BINDING SITE II and BINDING SITE III, inhibits translation of VGAM358 host target RNA into VGAM358 host target protein, herein designated VGAM HOST TARGET PROTEIN. VGAM host target protein is therefore outlined by a broken line.

It is appreciated that VGAM358 host target gene, herein designated VGAM HOST TARGET GENE, in fact represents a plurality of VGAM358 host target genes. The mRNA of each one of this plurality of VGAM358 host target genes comprises one or more host target binding sites, each having a nucleotide sequence which is at least partly complementary to VGAM358 RNA, herein designated VGAM RNA, and which when bound by VGAM358 RNA causes inhibition of translation of respective one or more VGAM358 host target proteins.

It is further appreciated by one skilled in the art that the mode of translational inhibition illustrated by FIG. 1 with specific reference to translational inhibition exerted by VGAM358 gene, herein designated VGAM GENE, on one or more VGAM358 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM358 is inhibition of expression of host target genes, as part of a novel viral mechanism of attacking a host. Accordingly, utilities of VGAM358 include diagnosis, prevention and treatment of viral infection by Vaccinia Virus. Specific functions, and accordingly utilities, of VGAM358 correlate with, and may be deduced from, the identity of the host target genes which VGAM358 binds and inhibits, and the function of these host target genes, as elaborated hereinbelow.

Nucleotide sequences of the VGAM358 precursor RNA, herein designated VGAM PRECURSOR RNA, and of the ‘diced’ VGAM358 RNA, herein designated VGAM RNA, and a schematic representation of the secondary folding of VGAM358 folded precursor RNA, herein designated VGAM FOLDED PRECURSOR RNA, of VGAM358 are further described hereinbelow with reference to Table 1.

Nucleotide sequences of host target binding sites, such as BINDING SITE-I, BINDING SITE-II and BINDING SITE-III of FIG. 1, found on VGAM358 host target RNA, and schematic representation of the complementarity of each of these host target binding sites to VGAM358 RNA, herein designated VGAM RNA, are described hereinbelow with reference to Table 2.

As mentioned hereinabove with reference to FIG. 1, a function of VGAM358 gene, herein designated VGAM is inhibition of expression of VGAM358 target genes. It is appreciated that specific functions, and accordingly utilities, of VGAM358 correlate with, and may be deduced from, the identity of the target genes which VGAM358 binds and inhibits, and the function of these target genes, as elaborated hereinbelow.

Ets Homologous Factor (EHF, Accession NM_{—}012153) is a VGAM358 host target gene. EHF BINDING SITE is HOST TARGET binding site found in the 3′ untranslated region of mRNA encoded by EHF, corresponding to a HOST TARGET binding site such as BINDING SITE I, BINDING SITE II or BINDING SITE III. Table 2 illustrates the complementarity of the nucleotide sequences of EHF BINDING SITE, designated SEQ ID:1411, to the nucleotide sequence of VGAM358 RNA, herein designated VGAM RNA, also designated SEQ ID:693.

A function of VGAM358 is therefore inhibition of Ets Homologous Factor (EHF, Accession NM_{—}012153), a gene which is Member of the ESE sub family of Ets transcription factors. Accordingly, utilities of VGAM358 include diagnosis, prevention and treatment of diseases and clinical conditions associated with EHF. The function of EHF has been established by previous studies. By searching an EST database, Kas et al. (2000) identified an EST with sequence similarity to the ETS domain of human ESE1 (ELF3; 602191). They obtained a full-length cDNA encoding EHF, which they called ESE3, by using 5-prime RACE on human prostate cDNA. RT-PCR analysis identified 2 alternatively spliced forms of ESE3, ESE3a and ESE3b. Sequence analysis predicted that ESE3a encodes a 277-amino acid protein with a molecular mass of 32.3 kD, while ESE3b encodes a 300-amino acid protein with a molecular mass of 34.9 kD. The C-terminal ETS domain of ESE3 is 84% and 65% identical to the ETS domains of ESE1 and ESE2 (ELF5; 605169), respectively. Northern blot analysis detected a 5.9-kb ESE3 transcript in pancreas and prostate, with lower levels detected in kidney and colon. Dot blot analysis detected high levels of ESE3 expression in salivary gland, prostate, and trachea, with lower levels detected in colon, mammary gland, pancreas, lung, stomach, appendix, fetal kidney, and fetal lung. Using RT-PCR on primary and tumor-derived cell lines, the authors detected expression of ESE3 in tumor cells of epithelial origin. Gel-shift experiments showed binding of ESE3 to 3 high-affinity binding sites in the MET (OMIM Ref. No. 164860) promoter. Cotransfection of ESE3 expression vectors with a MET promoter-luciferase reporter construct demonstrated that both ESE3a and ESE3b act as transcriptional activators on this promoter. Kleinbaum et al. (1999) mapped the EHF gene to 11p12 by somatic cell hybrid analysis and FISH.

Full details of the abovementioned studies are described in the following publications, the disclosure of which are hereby incorporated by reference:
Kas, K.; Finger, E.; Grall, F.; Gu, X.; Akbarali, Y.; Boltax, J.; Weiss, A.; Oettgen, P.; Kapeller, R.; Libermann, T. A.: ESE-3, a novel member of an epithelium-specific Ets transcription factor sub family, demonstrates different target gene specificity from ESE-1. J. Biol. Chem. 275: 2986-2998, 2000.; and
Kleinbaum, L. A.; Duggan, C.; Ferreira, E.; Coffey, G. P.; Buttice, G.; Burton, F. H.: Human chromosomal localization, tissue/tumor expression, and regulatory function of the ets fami.

Further studies establishing the function and utilities of EHF are found in John Hopkins OMIM database record ID 605439, and in sited publications numbered 1071-1072 listed in the bibliography section hereinbelow, which are also hereby incorporated by reference. Chromosome 9 Open Reading Frame 5 (C9orf5, Accession NM_{—}032012) is another VGAM358 host target gene. C9orf5 BINDING SITE is HOST TARGET binding site found in the 3′ untranslated region of mRNA encoded by C9orf5, corresponding to a HOST TARGET binding site such as BINDING SITE I, BINDING SITE II or BINDING SITE III. Table 2 illustrates the complementarity of the nucleotide sequences of C9orf5 BINDING SITE, designated SEQ ID:2225, to the nucleotide sequence of VGAM358 RNA, herein designated VGAM RNA, also designated SEQ ID:693.

Another function of VGAM358 is therefore inhibition of Chromosome 9 Open Reading Frame 5 (C9orf5, Accession NM_{—}032012). Accordingly, utilities of VGAM358 include diagnosis, prevention and treatment of diseases and clinical conditions associated with C9orf5. KIAA1546 (Accession XM_{—}042301) is another VGAM358 host target gene. KIAA1546 BINDING SITE is HOST TARGET binding site found in the 3′ untranslated region of mRNA encoded by KIAA1546, corresponding to a HOST TARGET binding site such as BINDING SITE I, BINDING SITE II or BINDING SITE III. Table 2 illustrates the complementarity of the nucleotide sequences of KIAA1546 BINDING SITE, designated SEQ ID:2805, to the nucleotide sequence of VGAM358 RNA, herein designated VGAM RNA, also designated SEQ ID:693.

Another function of VGAM358 is therefore inhibition of KIAA1546 (Accession XM_{—}042301). Accordingly, utilities of VGAM358 include diagnosis, prevention and treatment of diseases and clinical conditions associated with KIAA1546. LOC253263 (Accession XM_{—}173102) is another VGAM358 host target gene. LOC253263 BINDING SITE is HOST TARGET binding site found in the 3′ untranslated region of mRNA encoded by LOC253263, corresponding to a HOST TARGET binding site such as BINDING SITE I, BINDING SITE II or BINDING SITE III. Table 2 illustrates the complementarity of the nucleotide sequences of LOC253263 BINDING SITE, designated SEQ ID:3724, to the nucleotide sequence of VGAM358 RNA, herein designated VGAM RNA, also designated SEQ ID:693.

Another function of VGAM358 is therefore inhibition of LOC253263 (Accession XM_{—}173102). Accordingly, utilities of VGAM358 include diagnosis, prevention and treatment of diseases and clinical conditions associated with LOC253263. FIG. 1 further provides a conceptual description of a novel bioinformatically detected viral gene of the present invention, referred to here as Viral Genomic Address Messenger 359 (VGAM359) viral gene, which modulates expression of respective host target genes thereof, the function and utility of which host target genes is known in the art.

VGAM359 is a novel bioinformatically detected regulatory, non protein coding, viral micro RNA (miRNA) gene. The method by which VGAM359 was detected is described hereinabove with reference to FIGS. 1-8.

VGAM359 gene, herein designated VGAM GENE, is a viral gene contained in the genome of Vaccinia Virus. VGAM359 host target gene, herein designated VGAM HOST TARGET GENE, is a human gene contained in the human genome.

VGAM359 gene encodes a VGAM359 precursor RNA, herein designated VGAM PRECURSOR RNA. Similar to other miRNA genes, and unlike most ordinary genes, VGAM359 precursor RNA does not encode a protein. A nucleotide sequence identical or highly similar to the nucleotide sequence of VGAM359 precursor RNA is designated SEQ ID:345, and is provided hereinbelow with reference to the sequence listing part. Nucleotide sequence SEQ ID:345 is located at position 151270 relative to the genome of Vaccinia Virus.

VGAM359 precursor RNA folds onto itself, forming VGAM359 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM359 folded precursor RNA into VGAM359 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 84%) nucleotide sequence of VGAM359 RNA is designated SEQ ID:694, and is provided hereinbelow with reference to the sequence listing part.

VGAM359 host target gene, herein designated VGAM HOST TARGET GENE, encodes a corresponding messenger RNA, VGAM359 host target RNA, herein designated VGAM HOST TARGET RNA. VGAM359 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM359 RNA, herein designated VGAM RNA, binds complementarily to one or more host target binding sites located in untranslated regions of VGAM359 host target RNA, herein designated VGAM HOST TARGET RNA. This complementary binding is due to the fact that the nucleotide sequence of VGAM359 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM359 RNA, herein designated VGAM RNA, may have a different number of host target binding sites in untranslated regions of a VGAM359 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM359 RNA, herein designated VGAM RNA, to host target binding sites on VGAM359 host target RNA, herein designated VGAM HOST TARGET RNA, such as BINDING SITE I, BINDING SITE II and BINDING SITE III, inhibits translation of VGAM359 host target RNA into VGAM359 host target protein, herein designated VGAM HOST TARGET PROTEIN. VGAM host target protein is therefore outlined by a broken line.

It is appreciated that VGAM359 host target gene, herein designated VGAM HOST TARGET GENE, in fact represents a plurality of VGAM359 host target genes. The mRNA of each one of this plurality of VGAM359 host target genes comprises one or more host target binding sites, each having a nucleotide sequence which is at least partly complementary to VGAM359 RNA, herein designated VGAM RNA, and which when bound by VGAM359 RNA causes inhibition of translation of respective one or more VGAM359 host target proteins.

It is further appreciated by one skilled in the art that the mode of translational inhibition illustrated by FIG. 1 with specific reference to translational inhibition exerted by VGAM359 gene, herein designated VGAM GENE, on one or more VGAM359 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM359 is inhibition of expression of host target genes, as part of a novel viral mechanism of attacking a host. Accordingly, utilities of VGAM359 include diagnosis, prevention and treatment of viral infection by Vaccinia Virus. Specific functions, and accordingly utilities, of VGAM359 correlate with, and may be deduced from, the identity of the host target genes which VGAM359 binds and inhibits, and the function of these host target genes, as elaborated hereinbelow.

Nucleotide sequences of the VGAM359 precursor RNA, herein designated VGAM PRECURSOR RNA, and of the ‘diced’ VGAM359 RNA, herein designated VGAM RNA, and a schematic representation of the secondary folding of VGAM359 folded precursor RNA, herein designated VGAM FOLDED PRECURSOR RNA, of VGAM359 are further described hereinbelow with reference to Table 1.

Nucleotide sequences of host target binding sites, such as BINDING SITE-I, BINDING SITE-II and BINDING SITE-III of FIG. 1, found on VGAM359 host target RNA, and schematic representation of the complementarity of each of these host target binding sites to VGAM359 RNA, herein designated VGAM RNA, are described hereinbelow with reference to Table 2.

As mentioned hereinabove with reference to FIG. 1, a function of VGAM359 gene, herein designated VGAM is inhibition of expression of VGAM359 target genes. It is appreciated that specific functions, and accordingly utilities, of VGAM359 correlate with, and may be deduced from, the identity of the target genes which VGAM359 binds and inhibits, and the function of these target genes, as elaborated hereinbelow.

Breast Cancer 1, Early Onset (BRCA1, Accession NM_{—}007294) is a VGAM359 host target gene. BRCA1 BINDING SITE1 through BRCA1 BINDING SITE11 are HOST TARGET binding sites found in untranslated regions of mRNA encoded by BRCA1, corresponding to HOST TARGET binding sites such as BINDING SITE I, BINDING SITE II or BINDING SITE III. Table 2 illustrates the complementarity of the nucleotide sequences of BRCA1 BINDING SITE1 through BRCA1 BINDING SITE11, designated SEQ ID:1380, SEQ ID:1381, SEQ ID:1382, SEQ ID:1383, SEQ ID:1384, SEQ ID:1385, SEQ ID:1386, SEQ ID:1387, SEQ ID:1388, SEQ ID: 1389 and SEQ ID: 1391 respectively, to the nucleotide sequence of VGAM359 RNA, herein designated VGAM RNA, also designated SEQ ID:694.

A function of VGAM359 is therefore inhibition of Breast Cancer 1, Early Onset (BRCA1, Accession NM_{—}007294). Accordingly, utilities of VGAM359 include diagnosis, prevention and treatment of diseases and clinical conditions associated with BRCA1. Trichorhinophalangeal Syndrome I (TRPS1, Accession NM_{—}014112) is another VGAM359 host target gene. TRPS1 BINDING SITE is HOST TARGET binding site found in the 3′ untranslated region of mRNA encoded by TRPS1, corresponding to a HOST TARGET binding site such as BINDING SITE I, BINDING SITE II or BINDING SITE III. Table 2 illustrates the complementarity of the nucleotide sequences of TRPS1 BINDING SITE, designated SEQ ID:1470, to the nucleotide sequence of VGAM359 RNA, herein designated VGAM RNA, also designated SEQ ID:694.

Another function of VGAM359 is therefore inhibition of Trichorhinophalangeal Syndrome I (TRPS1, Accession NM_{—}014112), a gene which may function as a transcriptional activator protein. Accordingly, utilities of VGAM359 include diagnosis, prevention and treatment of diseases and clinical conditions associated with TRPS1. The function of TRPS1 and its association with various diseases and clinical conditions, has been established by previous studies, as described hereinabove with reference to VGAM49. FLJ23323 (Accession NM_{—}024654) is another VGAM359 host target gene. FLJ23323 BINDING SITE is HOST TARGET binding site found in the 3′ untranslated region of mRNA encoded by FLJ23323, corresponding to a HOST TARGET binding site such as BINDING SITE I, BINDING SITE II or BINDING SITE III. Table 2 illustrates the complementarity of the nucleotide sequences of FLJ23323 BINDING SITE, designated SEQ ID:2077, to the nucleotide sequence of VGAM359 RNA, herein designated VGAM RNA, also designated SEQ ID:694.

Another function of VGAM359 is therefore inhibition of FLJ23323 (Accession NM_{—}024654). Accordingly, utilities of VGAM359 include diagnosis, prevention and treatment of diseases and clinical conditions associated with FLJ23323. KIAA0057 (Accession NM_{—}012288) is another VGAM359 host target gene. KIAA0057 BINDING SITE is HOST TARGET binding site found in the 3′ untranslated region of mRNA encoded by KIAA0057, corresponding to a HOST TARGET binding site such as BINDING SITE I, BINDING SITE II or BINDING SITE III. Table 2 illustrates the complementarity of the nucleotide sequences of KIAA0057 BINDING SITE, designated SEQ ID:1421, to the nucleotide sequence of VGAM359 RNA, herein designated VGAM RNA, also designated SEQ ID:694.

Another function of VGAM359 is therefore inhibition of KIAA0057 (Accession NM_{—}012288). Accordingly, utilities of VGAM359 include diagnosis, prevention and treatment of diseases and clinical conditions associated with KIAA0057. LOC145439 (Accession XM_{—}085144) is another VGAM359 host target gene. LOC145439 BINDING SITE is HOST TARGET binding site found in the 5′ untranslated region of mRNA encoded by LOC145439, corresponding to a HOST TARGET binding site such as BINDING SITE I, BINDING SITE II or BINDING SITE III. Table 2 illustrates the complementarity of the nucleotide sequences of LOC145439 BINDING SITE, designated SEQ ID:3079, to the nucleotide sequence of VGAM359 RNA, herein designated VGAM RNA, also designated SEQ ID:694.

Another function of VGAM359 is therefore inhibition of LOC145439 (Accession XM_{—}085144). Accordingly, utilities of VGAM359 include diagnosis, prevention and treatment of diseases and clinical conditions associated with LOC145439. FIG. 1 further provides a conceptual description of a novel bioinformatically detected viral gene of the present invention, referred to here as Viral Genomic Address Messenger 360 (VGAM360) viral gene, which modulates expression of respective host target genes thereof, the function and utility of which host target genes is known in the art.

VGAM360 is a novel bioinformatically detected regulatory, non protein coding, viral micro RNA (miRNA) gene. The method by which VGAM360 was detected is described hereinabove with reference to FIGS. 1-8.

VGAM360 gene, herein designated VGAM GENE, is a viral gene contained in the genome of Vaccinia Virus. VGAM360 host target gene, herein designated VGAM HOST TARGET GENE, is a human gene contained in the human genome.

VGAM360 gene encodes a VGAM360 precursor RNA, herein designated VGAM PRECURSOR RNA. Similar to other miRNA genes, and unlike most ordinary genes, VGAM360 precursor RNA does not encode a protein. A nucleotide sequence identical or highly similar to the nucleotide sequence of VGAM360 precursor RNA is designated SEQ ID:346, and is provided hereinbelow with reference to the sequence listing part. Nucleotide sequence SEQ ID:346 is located at position 180452 relative to the genome of Vaccinia Virus.

VGAM360 precursor RNA folds onto itself, forming VGAM360 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM360 folded precursor RNA into VGAM360 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 85%) nucleotide sequence of VGAM360 RNA is designated SEQ ID:695, and is provided hereinbelow with reference to the sequence listing part.

VGAM360 host target gene, herein designated VGAM HOST TARGET GENE, encodes a corresponding messenger RNA, VGAM360 host target RNA, herein designated VGAM HOST TARGET RNA. VGAM360 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM360 RNA, herein designated VGAM RNA, binds complementarily to one or more host target binding sites located in untranslated regions of VGAM360 host target RNA, herein designated VGAM HOST TARGET RNA. This complementary binding is due to the fact that the nucleotide sequence of VGAM360 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM360 RNA, herein designated VGAM RNA, may have a different number of host target binding sites in untranslated regions of a VGAM360 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM360 RNA, herein designated VGAM RNA, to host target binding sites on VGAM360 host target RNA, herein designated VGAM HOST TARGET RNA, such as BINDING SITE I, BINDING SITE II and BINDING SITE III, inhibits translation of VGAM360 host target RNA into VGAM360 host target protein, herein designated VGAM HOST TARGET PROTEIN. VGAM host target protein is therefore outlined by a broken line.

It is appreciated that VGAM360 host target gene, herein designated VGAM HOST TARGET GENE, in fact represents a plurality of VGAM360 host target genes. The mRNA of each one of this plurality of VGAM360 host target genes comprises one or more host target binding sites, each having a nucleotide sequence which is at least partly complementary to VGAM360 RNA, herein designated VGAM RNA, and which when bound by VGAM360 RNA causes inhibition of translation of respective one or more VGAM360 host target proteins.

It is further appreciated by one skilled in the art that the mode of translational inhibition illustrated by FIG. 1 with specific reference to translational inhibition exerted by VGAM360 gene, herein designated VGAM GENE, on one or more VGAM360 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM360 is inhibition of expression of host target genes, as part of a novel viral mechanism of attacking a host. Accordingly, utilities of VGAM360 include diagnosis, prevention and treatment of viral infection by Vaccinia Virus. Specific functions, and accordingly utilities, of VGAM360 correlate with, and may be deduced from, the identity of the host target genes which VGAM360 binds and inhibits, and the function of these host target genes, as elaborated hereinbelow.

Nucleotide sequences of the VGAM360 precursor RNA, herein designated VGAM PRECURSOR RNA, and of the ‘diced’ VGAM360 RNA, herein designated VGAM RNA, and a schematic representation of the secondary folding of VGAM360 folded precursor RNA, herein designated VGAM FOLDED PRECURSOR RNA, of VGAM360 are further described hereinbelow with reference to Table 1.

Nucleotide sequences of host target binding sites, such as BINDING SITE-I, BINDING SITE-II and BINDING SITE-III of FIG. 1, found on VGAM360 host target RNA, and schematic representation of the complementarity of each of these host target binding sites to VGAM360 RNA, herein designated VGAM RNA, are described hereinbelow with reference to Table 2.

As mentioned hereinabove with reference to FIG. 1, a function of VGAM360 gene, herein designated VGAM is inhibition of expression of VGAM360 target genes. It is appreciated that specific functions, and accordingly utilities, of VGAM360 correlate with, and may be deduced from, the identity of the target genes which VGAM360 binds and inhibits, and the function of these target genes, as elaborated hereinbelow.

DKFZP434G1411 (Accession XM_{—}166383) is a VGAM360 host target gene. DKFZP434G1411 BINDING SITE is HOST TARGET binding site found in the 3′ untranslated region of mRNA encoded by DKFZP434G1411, corresponding to a HOST TARGET binding site such as BINDING SITE I, BINDING SITE II or BINDING SITE III. Table 2 illustrates the complementarity of the nucleotide sequences of DKFZP434G1411 BINDING SITE, designated SEQ ID:3554, to the nucleotide sequence of VGAM360 RNA, herein designated VGAM RNA, also designated SEQ ID:695.

A function of VGAM360 is therefore inhibition of DKFZP434G1411 (Accession XM_{—}166383). Accordingly, utilities of VGAM360 include diagnosis, prevention and treatment of diseases and clinical conditions associated with DKFZP434G1411. DKFZP5641052 (Accession XM_{—}039660) is another VGAM360 host target gene. DKFZP5641052 BINDING SITE is HOST TARGET binding site found in the 3′ untranslated region of mRNA encoded by DKFZP5641052, corresponding to a HOST TARGET binding site such as BINDING SITE I, BINDING SITE II or BINDING SITE III. Table 2 illustrates the complementarity of the nucleotide sequences of DKFZP5641052 BINDING SITE, designated SEQ ID:2760, to the nucleotide sequence of VGAM360 RNA, herein designated VGAM RNA, also designated SEQ ID:695.

Another function of VGAM360 is therefore inhibition of DKFZP5641052 (Accession XM_{—}039660). Accordingly, utilities of VGAM360 include diagnosis, prevention and treatment of diseases and clinical conditions associated with DKFZP5641052. Kelch-like 8 (Drosophila) (KLHL8, Accession XM_{—}031735) is another VGAM360 host target gene. KLHL8 BINDING SITE is HOST TARGET binding site found in the 3′ untranslated region of mRNA encoded by KLHL8, corresponding to a HOST TARGET binding site such as BINDING SITE I, BINDING SITE II or BINDING SITE III. Table 2 illustrates the complementarity of the nucleotide sequences of KLHL8 BINDING SITE, designated SEQ ID:2639, to the nucleotide sequence of VGAM360 RNA, herein designated VGAM RNA, also designated SEQ ID:695.

Another function of VGAM360 is therefore inhibition of Kelch-like 8 (Drosophila) (KLHL8, Accession XM_{—}031735). Accordingly, utilities of VGAM360 include diagnosis, prevention and treatment of diseases and clinical conditions associated with KLHL8. Extracellular Link Domain Containing 1 (XLKD1, Accession NM_{—}006691) is another VGAM360 host target gene. XLKD1 BINDING SITE is HOST TARGET binding site found in the 3′ untranslated region of mRNA encoded by XLKD1, corresponding to a HOST TARGET binding site such as BINDING SITE I, BINDING SITE II or BINDING SITE III. Table 2 illustrates the complementarity of the nucleotide sequences of XLKD1 BINDING SITE, designated SEQ ID:1323, to the nucleotide sequence of VGAM360 RNA, herein designated VGAM RNA, also designated SEQ ID:695.

Another function of VGAM360 is therefore inhibition of Extracellular Link Domain Containing 1 (XLKD1, Accession NM_{—}006691). Accordingly, utilities of VGAM360 include diagnosis, prevention and treatment of diseases and clinical conditions associated with XLKD1. LOC51003 (Accession NM_{—}016060) is another VGAM360 host target gene. LOC51003 BINDING SITE is HOST TARGET binding site found in the 3′ untranslated region of mRNA encoded by LOC51003, corresponding to a HOST TARGET binding site such as BINDING SITE I, BINDING SITE II or BINDING SITE III. Table 2 illustrates the complementarity of the nucleotide sequences of LOC51003 BINDING SITE, designated SEQ ID: 1658, to the nucleotide sequence of VGAM360 RNA, herein designated VGAM RNA, also designated SEQ ID:695.

Another function of VGAM360 is therefore inhibition of LOC51003 (Accession NM_{—}016060). Accordingly, utilities of VGAM360 include diagnosis, prevention and treatment of diseases and clinical conditions associated with LOC51003. FIG. 1 further provides a conceptual description of a novel bioinformatically detected viral gene of the present invention, referred to here as Viral Genomic Address Messenger 361 (VGAM361) viral gene, which modulates expression of respective host target genes thereof, the function and utility of which host target genes is known in the art.

VGAM361 is a novel bioinformatically detected regulatory, non protein coding, viral micro RNA (miRNA) gene. The method by which VGAM361 was detected is described hereinabove with reference to FIGS. 1-8.

VGAM361 gene, herein designated VGAM GENE, is a viral gene contained in the genome of Vaccinia Virus. VGAM361 host target gene, herein designated VGAM HOST TARGET GENE, is a human gene contained in the human genome.

VGAM361 gene encodes a VGAM361 precursor RNA, herein designated VGAM PRECURSOR RNA. Similar to other miRNA genes, and unlike most ordinary genes, VGAM361 precursor RNA does not encode a protein. A nucleotide sequence identical or highly similar to the nucleotide sequence of VGAM361 precursor RNA is designated SEQ ID:347, and is provided hereinbelow with reference to the sequence listing part. Nucleotide sequence SEQ ID:347 is located at position 183817 relative to the genome of Vaccinia Virus.

VGAM361 precursor RNA folds onto itself, forming VGAM361 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM361 folded precursor RNA into VGAM361 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 80%) nucleotide sequence of VGAM361 RNA is designated SEQ ID:696, and is provided hereinbelow with reference to the sequence listing part.

VGAM361 host target gene, herein designated VGAM HOST TARGET GENE, encodes a corresponding messenger RNA, VGAM361 host target RNA, herein designated VGAM HOST TARGET RNA. VGAM361 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM361 RNA, herein designated VGAM RNA, binds complementarily to one or more host target binding sites located in untranslated regions of VGAM361 host target RNA, herein designated VGAM HOST TARGET RNA. This complementary binding is due to the fact that the nucleotide sequence of VGAM361 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM361 RNA, herein designated VGAM RNA, may have a different number of host target binding sites in untranslated regions of a VGAM361 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM361 RNA, herein designated VGAM RNA, to host target binding sites on VGAM361 host target RNA, herein designated VGAM HOST TARGET RNA, such as BINDING SITE I, BINDING SITE II and BINDING SITE III, inhibits translation of VGAM361 host target RNA into VGAM361 host target protein, herein designated VGAM HOST TARGET PROTEIN. VGAM host target protein is therefore outlined by a broken line.

It is appreciated that VGAM361 host target gene, herein designated VGAM HOST TARGET GENE, in fact represents a plurality of VGAM361 host target genes. The mRNA of each one of this plurality of VGAM361 host target genes comprises one or more host target binding sites, each having a nucleotide sequence which is at least partly complementary to VGAM361 RNA, herein designated VGAM RNA, and which when bound by VGAM361 RNA causes inhibition of translation of respective one or more VGAM361 host target proteins.

It is further appreciated by one skilled in the art that the mode of translational inhibition illustrated by FIG. 1 with specific reference to translational inhibition exerted by VGAM361 gene, herein designated VGAM GENE, on one or more VGAM361 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM361 is inhibition of expression of host target genes, as part of a novel viral mechanism of attacking a host. Accordingly, utilities of VGAM361 include diagnosis, prevention and treatment of viral infection by Vaccinia Virus. Specific functions, and accordingly utilities, of VGAM361 correlate with, and may be deduced from, the identity of the host target genes which VGAM361 binds and inhibits, and the function of these host target genes, as elaborated hereinbelow.

Nucleotide sequences of the VGAM361 precursor RNA, herein designated VGAM PRECURSOR RNA, and of the ‘diced’ VGAM361 RNA, herein designated VGAM RNA, and a schematic representation of the secondary folding of VGAM361 folded precursor RNA, herein designated VGAM FOLDED PRECURSOR RNA, of VGAM361 are further described hereinbelow with reference to Table 1.

Nucleotide sequences of host target binding sites, such as BINDING SITE-I, BINDING SITE-II and BINDING SITE-III of FIG. 1, found on VGAM361 host target RNA, and schematic representation of the complementarity of each of these host target binding sites to VGAM361 RNA, herein designated VGAM RNA, are described hereinbelow with reference to Table 2.

As mentioned hereinabove with reference to FIG. 1, a function of VGAM361 gene, herein designated VGAM is inhibition of expression of VGAM361 target genes. It is appreciated that specific functions, and accordingly utilities, of VGAM361 correlate with, and may be deduced from, the identity of the target genes which VGAM361 binds and inhibits, and the function of these target genes, as elaborated hereinbelow.

Glucosaminyl (N-acetyl) Transferase 2,1-branching Enzyme (GCNT2, Accession NM_{—}001491) is a VGAM361 host target gene. GCNT2 BINDING SITE is HOST TARGET binding site found in the 5′ untranslated region of mRNA encoded by GCNT2, corresponding to a HOST TARGET binding site such as BINDING SITE I, BINDING SITE II or BINDING SITE III. Table 2 illustrates the complementarity of the nucleotide sequences of GCNT2 BINDING SITE, designated SEQ ID:831, to the nucleotide sequence of VGAM361 RNA, herein designated VGAM RNA, also designated SEQ ID:696.

A function of VGAM361 is therefore inhibition of Glucosaminyl (N-acetyl) Transferase 2,1-branching Enzyme (GCNT2, Accession NM_{—}001491), a gene which converts linear into branched poly-n-acetyllactosaminoglycans. Accordingly, utilities of VGAM361 include diagnosis, prevention and treatment of diseases and clinical conditions associated with GCNT2. The function of GCNT2 and its association with various diseases and clinical conditions, has been established by previous studies, as described hereinabove with reference to VGAM56. BLP1 (Accession NM_{—}031940) is another VGAM361 host target gene. BLP1 BINDING SITE1 and BLP1 BINDING SITE2 are HOST TARGET binding sites found in untranslated regions of mRNA encoded by BLP1, corresponding to HOST TARGET binding sites such as BINDING SITE I, BINDING SITE II or BINDING SITE III. Table 2 illustrates the complementarity of the nucleotide sequences of BLP1 BINDING SITE1 and BLP1 BINDING SITE2, designated SEQ ID:2222 and SEQ ID:2371 respectively, to the nucleotide sequence of VGAM361 RNA, herein designated VGAM RNA, also designated SEQ ID:696.

Another function of VGAM361 is therefore inhibition of BLP1 (Accession NM_{—}031940). Accordingly, utilities of VGAM361 include diagnosis, prevention and treatment of diseases and clinical conditions associated with BLP1. LOC253891 (Accession XM_{—}170485) is another VGAM361 host target gene. LOC253891 BINDING SITE is HOST TARGET binding site found in the 5′ untranslated region of mRNA encoded by LOC253891, corresponding to a HOST TARGET binding site such as BINDING SITE I, BINDING SITE II or BINDING SITE III. Table 2 illustrates the complementarity of the nucleotide sequences of LOC253891 BINDING SITE, designated SEQ ID:3654, to the nucleotide sequence of VGAM361 RNA, herein designated VGAM RNA, also designated SEQ ID:696.

Another function of VGAM361 is therefore inhibition of LOC253891 (Accession XM_{—}170485). Accordingly, utilities of VGAM361 include diagnosis, prevention and treatment of diseases and clinical conditions associated with LOC253891. FIG. 1 further provides a conceptual description of a novel bioinformatically detected viral gene of the present invention, referred to here as Viral Genomic Address Messenger 362 (VGAM362) viral gene, which modulates expression of respective host target genes thereof, the function and utility of which host target genes is known in the art.

VGAM362 is a novel bioinformatically detected regulatory, non protein coding, viral micro RNA (miRNA) gene. The method by which VGAM362 was detected is described hereinabove with reference to FIGS. 1-8.

VGAM362 gene, herein designated VGAM GENE, is a viral gene contained in the genome of Vaccinia Virus. VGAM362 host target gene, herein designated VGAM HOST TARGET GENE, is a human gene contained in the human genome.

VGAM362 gene encodes a VGAM362 precursor RNA, herein designated VGAM PRECURSOR RNA. Similar to other miRNA genes, and unlike most ordinary genes, VGAM362 precursor RNA does not encode a protein. A nucleotide sequence identical or highly similar to the nucleotide sequence of VGAM362 precursor RNA is designated SEQ ID:348, and is provided hereinbelow with reference to the sequence listing part. Nucleotide sequence SEQ ID:348 is located at position 183735 relative to the genome of Vaccinia Virus.

VGAM362 precursor RNA folds onto itself, forming VGAM362 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM362 folded precursor RNA into VGAM362 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 71%) nucleotide sequence of VGAM362 RNA is designated SEQ ID:697, and is provided hereinbelow with reference to the sequence listing part.

VGAM362 host target gene, herein designated VGAM HOST TARGET GENE, encodes a corresponding messenger RNA, VGAM362 host target RNA, herein designated VGAM HOST TARGET RNA. VGAM362 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM362 RNA, herein designated VGAM RNA, binds complementarily to one or more host target binding sites located in untranslated regions of VGAM362 host target RNA, herein designated VGAM HOST TARGET RNA. This complementary binding is due to the fact that the nucleotide sequence of VGAM362 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM362 RNA, herein designated VGAM RNA, may have a different number of host target binding sites in untranslated regions of a VGAM362 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM362 RNA, herein designated VGAM RNA, to host target binding sites on VGAM362 host target RNA, herein designated VGAM HOST TARGET RNA, such as BINDING SITE I, BINDING SITE II and BINDING SITE III, inhibits translation of VGAM362 host target RNA into VGAM362 host target protein, herein designated VGAM HOST TARGET PROTEIN. VGAM host target protein is therefore outlined by a broken line.

It is appreciated that VGAM362 host target gene, herein designated VGAM HOST TARGET GENE, in fact represents a plurality of VGAM362 host target genes. The mRNA of each one of this plurality of VGAM362 host target genes comprises one or more host target binding sites, each having a nucleotide sequence which is at least partly complementary to VGAM362 RNA, herein designated VGAM RNA, and which when bound by VGAM362 RNA causes inhibition of translation of respective one or more VGAM362 host target proteins.

It is further appreciated by one skilled in the art that the mode of translational inhibition illustrated by FIG. 1 with specific reference to translational inhibition exerted by VGAM362 gene, herein designated VGAM GENE, on one or more VGAM362 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM362 is inhibition of expression of host target genes, as part of a novel viral mechanism of attacking a host. Accordingly, utilities of VGAM362 include diagnosis, prevention and treatment of viral infection by Vaccinia Virus. Specific functions, and accordingly utilities, of VGAM362 correlate with, and may be deduced from, the identity of the host target genes which VGAM362 binds and inhibits, and the function of these host target genes, as elaborated hereinbelow.

Nucleotide sequences of the VGAM362 precursor RNA, herein designated VGAM PRECURSOR RNA, and of the ‘diced’ VGAM362 RNA, herein designated VGAM RNA, and a schematic representation of the secondary folding of VGAM362 folded precursor RNA, herein designated VGAM FOLDED PRECURSOR RNA, of VGAM362 are further described hereinbelow with reference to Table 1.

Nucleotide sequences of host target binding sites, such as BINDING SITE-I, BINDING SITE-II and BINDING SITE-III of FIG. 1, found on VGAM362 host target RNA, and schematic representation of the complementarity of each of these host target binding sites to VGAM362 RNA, herein designated VGAM RNA, are described hereinbelow with reference to Table 2.

As mentioned hereinabove with reference to FIG. 1, a function of VGAM362 gene, herein designated VGAM is inhibition of expression of VGAM362 target genes. It is appreciated that specific functions, and accordingly utilities, of VGAM362 correlate with, and may be deduced from, the identity of the target genes which VGAM362 binds and inhibits, and the function of these target genes, as elaborated hereinbelow.

Adenylosuccinate Synthase (ADSS, Accession XM_{—}049992) is a VGAM362 host target gene. ADSS BINDING SITE is HOST TARGET binding site found in the 3′ untranslated region of mRNA encoded by ADSS, corresponding to a HOST TARGET binding site such as BINDING SITE I, BINDING SITE II or BINDING SITE III. Table 2 illustrates the complementarity of the nucleotide sequences of ADSS BINDING SITE, designated SEQ ID:2925, to the nucleotide sequence of VGAM362 RNA, herein designated VGAM RNA, also designated SEQ ID:697.

A function of VGAM362 is therefore inhibition of Adenylosuccinate Synthase (ADSS, Accession XM_{—}049992), a gene which plays an important role in the de novo pathway of purine nucleotide biosynthesis. Accordingly, utilities of VGAM362 include diagnosis, prevention and treatment of diseases and clinical conditions associated with ADSS. The function of ADSS and its association with various diseases and clinical conditions, has been established by previous studies, as described hereinabove with reference to VGAM57. Caspase 6, Apoptosis-related Cysteine Protease (CASP6, Accession NM_{—}032992) is another VGAM362 host target gene. CASP6 BINDING SITE1 and CASP6 BINDING SITE2 are HOST TARGET binding sites found in untranslated regions of mRNA encoded by CASP6, corresponding to HOST TARGET binding sites such as BINDING SITE I, BINDING SITE II or BINDING SITE III. Table 2 illustrates the complementarity of the nucleotide sequences of CASP6 BINDING SITE1 and CASP6 BINDING SITE2, designated SEQ ID:2305 and SEQ ID:810 respectively, to the nucleotide sequence of VGAM362 RNA, herein designated VGAM RNA, also designated SEQ ID:697.

Another function of VGAM362 is therefore inhibition of Caspase 6, Apoptosis-related Cysteine Protease (CASP6, Accession NM_{—}032992), a gene which involves in the activation cascade of caspases responsible for apoptosis execution. Accordingly, utilities of VGAM362 include diagnosis, prevention and treatment of diseases and clinical conditions associated with CASP6. The function of CASP6 and its association with various diseases and clinical conditions, has been established by previous studies, as described hereinabove with reference to VGAM57. Coagulation Factor II (thrombin) Receptor (F2R, Accession NM_{—}001992) is another VGAM362 host target gene. F2R BINDING SITE is HOST TARGET binding site found in the 3′ untranslated region of mRNA encoded by F2R, corresponding to a HOST TARGET binding site such as BINDING SITE I, BINDING SITE II or BINDING SITE III. Table 2 illustrates the complementarity of the nucleotide sequences of F2R BINDING SITE, designated SEQ ID:879, to the nucleotide sequence of VGAM362 RNA, herein designated VGAM RNA, also designated SEQ ID:697.

Another function of VGAM362 is therefore inhibition of Coagulation Factor II (thrombin) Receptor (F2R, Accession NM_{—}001992), a gene which Thrombin receptor; G protein-coupled receptor involved in platelet activation. Accordingly, utilities of VGAM362 include diagnosis, prevention and treatment of diseases and clinical conditions associated with F2R. The function of F2R and its association with various diseases and clinical conditions, has been established by previous studies, as described hereinabove with reference to VGAM57. G Protein-coupled Receptor Kinase 7 (GPRK7, Accession NM_{—}139209) is another VGAM362 host target gene. GPRK7 BINDING SITE is HOST TARGET binding site found in the 3′ untranslated region of mRNA encoded by GPRK7, corresponding to a HOST TARGET binding site such as BINDING SITE I, BINDING SITE II or BINDING SITE III. Table 2 illustrates the complementarity of the nucleotide sequences of GPRK7 BINDING SITE, designated SEQ ID:2476, to the nucleotide sequence of VGAM362 RNA, herein designated VGAM RNA, also designated SEQ ID:697.

Another function of VGAM362 is therefore inhibition of G Protein-coupled Receptor Kinase 7 (GPRK7, Accession NM_{—}139209), a gene which may play a role in signal transduction pathways that involve calcium as a second messenger. Accordingly, utilities of VGAM362 include diagnosis, prevention and treatment of diseases and clinical conditions associated with GPRK7. The function of GPRK7 and its association with various diseases and clinical conditions, has been established by previous studies, as described hereinabove with reference to VGAM57. Neuroligin 1 (NLGN1, Accession NM_{—}014932) is another VGAM362 host target gene. NLGN1 BINDING SITE is HOST TARGET binding site found in the 3′ untranslated region of mRNA encoded by NLGN1, corresponding to a HOST TARGET binding site such as BINDING SITE I, BINDING SITE II or BINDING SITE III. Table 2 illustrates the complementarity of the nucleotide sequences of NLGN1 BINDING SITE, designated SEQ ID:1591, to the nucleotide sequence of VGAM362 RNA, herein designated VGAM RNA, also designated SEQ ID:697.

Another function of VGAM362 is therefore inhibition of Neuroligin 1 (NLGN1, Accession NM_{—}014932), a gene which may trigger the de novo formation of presynaptic structure. Accordingly, utilities of VGAM362 include diagnosis, prevention and treatment of diseases and clinical conditions associated with NLGN1. The function of NLGN1 and its association with various diseases and clinical conditions, has been established by previous studies, as described hereinabove with reference to VGAM57. APC10 (Accession NM_{—}014885) is another VGAM362 host target gene. APC10 BINDING SITE is HOST TARGET binding site found in the 5′ untranslated region of mRNA encoded by APC10, corresponding to a HOST TARGET binding site such as BINDING SITE I, BINDING SITE II or BINDING SITE III. Table 2 illustrates the complementarity of the nucleotide sequences of APC10 BINDING SITE, designated SEQ ID:1574, to the nucleotide sequence of VGAM362 RNA, herein designated VGAM RNA, also designated SEQ ID:697.

Another function of VGAM362 is therefore inhibition of APC10 (Accession NM_{—}014885). Accordingly, utilities of VGAM362 include diagnosis, prevention and treatment of diseases and clinical conditions associated with APC10. Chromosome 20 Open Reading Frame 139 (C20orf139, Accession XM_{—}097749) is another VGAM362 host target gene. C20orf139 BINDING SITE is HOST TARGET binding site found in the 3′ untranslated region of mRNA encoded by C20orf139, corresponding to a HOST TARGET binding site such as BINDING SITE I, BINDING SITE II or BINDING SITE III. Table 2 illustrates the complementarity of the nucleotide sequences of C20orf139 BINDING SITE, designated SEQ ID:3319, to the nucleotide sequence of VGAM362 RNA, herein designated VGAM RNA, also designated SEQ ID:697.

Another function of VGAM362 is therefore inhibition of Chromosome 20 Open Reading Frame 139 (C20orf139, Accession XM_{—}097749). Accordingly, utilities of VGAM362 include diagnosis, prevention and treatment of diseases and clinical conditions associated with C20orf139. FLJ11186 (Accession NM_{—}018353) is another VGAM362 host target gene. FLJ11186 BINDING SITE is HOST TARGET binding site found in the 3′ untranslated region of mRNA encoded by FLJ11186, corresponding to a HOST TARGET binding site such as BINDING SITE I, BINDING SITE II or BINDING SITE III. Table 2 illustrates the complementarity of the nucleotide sequences of FLJ11186 BINDING SITE, designated SEQ ID:1818, to the nucleotide sequence of VGAM362 RNA, herein designated VGAM RNA, also designated SEQ ID:697.

Another function of VGAM362 is therefore inhibition of FLJ11186 (Accession NM_{—}018353). Accordingly, utilities of VGAM362 include diagnosis, prevention and treatment of diseases and clinical conditions associated with FLJ11186. FLJ22116 (Accession NM_{—}024624) is another VGAM362 host target gene. FLJ22116 BINDING SITE is HOST TARGET binding site found in the 5′ untranslated region of mRNA encoded by FLJ22116, corresponding to a HOST TARGET binding site such as BINDING SITE I, BINDING SITE II or BINDING SITE III. Table 2 illustrates the complementarity of the nucleotide sequences of FLJ22116 BINDING SITE, designated SEQ ID:2073, to the nucleotide sequence of VGAM362 RNA, herein designated VGAM RNA, also designated SEQ ID:697.

Another function of VGAM362 is therefore inhibition of FLJ22116 (Accession NM_{—}024624). Accordingly, utilities of VGAM362 include diagnosis, prevention and treatment of diseases and clinical conditions associated with FLJ22116. FLJ32978 (Accession NM_{—}144625) is another VGAM362 host target gene. FLJ32978 BINDING SITE is HOST TARGET binding site found in the 3′ untranslated region of mRNA encoded by FLJ32978, corresponding to a HOST TARGET binding site such as BINDING SITE I, BINDING SITE II or BINDING SITE III. Table 2 illustrates the complementarity of the nucleotide sequences of FLJ32978 BINDING SITE, designated SEQ ID:2487, to the nucleotide sequence of VGAM362 RNA, herein designated VGAM RNA, also designated SEQ ID:697.

Another function of VGAM362 is therefore inhibition of FLJ32978 (Accession NM_{—}144625). Accordingly, utilities of VGAM362 include diagnosis, prevention and treatment of diseases and clinical conditions associated with FLJ32978. KIAA1164 (Accession XM_{—}045358) is another VGAM362 host target gene. KIAA1164 BINDING SITE is HOST TARGET binding site found in the 3′ untranslated region of mRNA encoded by KIAA1164, corresponding to a HOST TARGET binding site such as BINDING SITE I, BINDING SITE II or BINDING SITE III. Table 2 illustrates the complementarity of the nucleotide sequences of KIAA1164 BINDING SITE, designated SEQ ID:2862, to the nucleotide sequence of VGAM362 RNA, herein designated VGAM RNA, also designated SEQ ID:697.

Another function of VGAM362 is therefore inhibition of KIAA1164 (Accession XM_{—}045358). Accordingly, utilities of VGAM362 include diagnosis, prevention and treatment of diseases and clinical conditions associated with KIAA1164. KIAA1762 (Accession XM_{—}033370) is another VGAM362 host target gene. KIAA1762 BINDING SITE is HOST TARGET binding site found in the 3′ untranslated region of mRNA encoded by KIAA1762, corresponding to a HOST TARGET binding site such as BINDING SITE I, BINDING SITE II or BINDING SITE III. Table 2 illustrates the complementarity of the nucleotide sequences of KIAA1762 BINDING SITE, designated SEQ ID:2668, to the nucleotide sequence of VGAM362 RNA, herein designated VGAM RNA, also designated SEQ ID:697.

Another function of VGAM362 is therefore inhibition of KIAA1762 (Accession XM_{—}033370). Accordingly, utilities of VGAM362 include diagnosis, prevention and treatment of diseases and clinical conditions associated with KIAA1762. KIAA1918 (Accession XM_{—}054951) is another VGAM362 host target gene. KIAA1918 BINDING SITE is HOST TARGET binding site found in the 5′ untranslated region of mRNA encoded by KIAA1918, corresponding to a HOST TARGET binding site such as BINDING SITE I, BINDING SITE II or BINDING SITE III. Table 2 illustrates the complementarity of the nucleotide sequences of KIAA1918 BINDING SITE, designated SEQ ID:2969, to the nucleotide sequence of VGAM362 RNA, herein designated VGAM RNA, also designated SEQ ID:697.

Another function of VGAM362 is therefore inhibition of KIAA1918 (Accession XM_{—}054951). Accordingly, utilities of VGAM362 include diagnosis, prevention and treatment of diseases and clinical conditions associated with KIAA1918. POPX1 (Accession NM_{—}014906) is another VGAM362 host target gene. POPX1 BINDING SITE is HOST TARGET binding site found in the 3′ untranslated region of mRNA encoded by POPX1, corresponding to a HOST TARGET binding site such as BINDING SITE I, BINDING SITE II or BINDING SITE III. Table 2 illustrates the complementarity of the nucleotide sequences of POPX1 BINDING SITE, designated SEQ ID:1583, to the nucleotide sequence of VGAM362 RNA, herein designated VGAM RNA, also designated SEQ ID:697.

Another function of VGAM362 is therefore inhibition of POPX1 (Accession NM_{—}014906). Accordingly, utilities of VGAM362 include diagnosis, prevention and treatment of diseases and clinical conditions associated with POPX1. PRO0800 (Accession NM_{—}018592) is another VGAM362 host target gene. PRO0800 BINDING SITE is HOST TARGET binding site found in the 3′ untranslated region of mRNA encoded by PRO0800, corresponding to a HOST TARGET binding site such as BINDING SITE I, BINDING SITE II or BINDING SITE III. Table 2 illustrates the complementarity of the nucleotide sequences of PRO0800 BINDING SITE, designated SEQ ID:1852, to the nucleotide sequence of VGAM362 RNA, herein designated VGAM RNA, also designated SEQ ID:697.

Another function of VGAM362 is therefore inhibition of PRO0800 (Accession NM_{—}018592). Accordingly, utilities of VGAM362 include diagnosis, prevention and treatment of diseases and clinical conditions associated with PRO0800. LOC142779 (Accession XM_{—}084337) is another VGAM362 host target gene. LOC142779 BINDING SITE is HOST TARGET binding site found in the 3′ untranslated region of mRNA encoded by LOC142779, corresponding to a HOST TARGET binding site such as BINDING SITE I, BINDING SITE II or BINDING SITE III. Table 2 illustrates the complementarity of the nucleotide sequences of LOC142779 BINDING SITE, designated SEQ ID:3050, to the nucleotide sequence of VGAM362 RNA, herein designated VGAM RNA, also designated SEQ ID:697.

Another function of VGAM362 is therefore inhibition of LOC142779 (Accession XM_{—}084337). Accordingly, utilities of VGAM362 include diagnosis, prevention and treatment of diseases and clinical conditions associated with LOC142779. LOC143274 (Accession XM_{—}084477) is another VGAM362 host target gene. LOC143274 BINDING SITE is HOST TARGET binding site found in the 5′ untranslated region of mRNA encoded by LOC143274, corresponding to a HOST TARGET binding site such as BINDING SITE I, BINDING SITE II or BINDING SITE III. Table 2 illustrates the complementarity of the nucleotide sequences of LOC143274 BINDING SITE, designated SEQ ID:3053, to the nucleotide sequence of VGAM362 RNA, herein designated VGAM RNA, also designated SEQ ID:697.

Another function of VGAM362 is therefore inhibition of LOC143274 (Accession XM_{—}084477). Accordingly, utilities of VGAM362 include diagnosis, prevention and treatment of diseases and clinical conditions associated with LOC143274. LOC154089 (Accession XM_{—}087846) is another VGAM362 host target gene. LOC154089 BINDING SITE is HOST TARGET binding site found in the 3′ untranslated region of mRNA encoded by LOC154089, corresponding to a HOST TARGET binding site such as BINDING SITE I, BINDING SITE II or BINDING SITE III. Table 2 illustrates the complementarity of the nucleotide sequences of LOC154089 BINDING SITE, designated SEQ ID:3186, to the nucleotide sequence of VGAM362 RNA, herein designated VGAM RNA, also designated SEQ ID:697.

Another function of VGAM362 is therefore inhibition of LOC154089 (Accession XM_{—}087846). Accordingly, utilities of VGAM362 include diagnosis, prevention and treatment of diseases and clinical conditions associated with LOC154089. LOC169679 (Accession XM_{—}108982) is another VGAM362 host target gene. LOC169679 BINDING SITE is HOST TARGET binding site found in the 3′ untranslated region of mRNA encoded by LOC169679, corresponding to a HOST TARGET binding site such as BINDING SITE I, BINDING SITE II or BINDING SITE III. Table 2 illustrates the complementarity of the nucleotide sequences of LOC169679 BINDING SITE, designated SEQ ID:3404, to the nucleotide sequence of VGAM362 RNA, herein designated VGAM RNA, also designated SEQ ID:697.

Another function of VGAM362 is therefore inhibition of LOC169679 (Accession XM_{—}108982). Accordingly, utilities of VGAM362 include diagnosis, prevention and treatment of diseases and clinical conditions associated with LOC169679. LOC202052 (Accession XM_{—}117355) is another VGAM362 host target gene. LOC202052 BINDING SITE is HOST TARGET binding site found in the 5′ untranslated region of mRNA encoded by LOC202052, corresponding to a HOST TARGET binding site such as BINDING SITE I, BINDING SITE II or BINDING SITE III. Table 2 illustrates the complementarity of the nucleotide sequences of LOC202052 BINDING SITE, designated SEQ ID:3484, to the nucleotide sequence of VGAM362 RNA, herein designated VGAM RNA, also designated SEQ ID:697.

Another function of VGAM362 is therefore inhibition of LOC202052 (Accession XM_{—}117355). Accordingly, utilities of VGAM362 include diagnosis, prevention and treatment of diseases and clinical conditions associated with LOC202052. LOC256021 (Accession XM_{—}172884) is another VGAM362 host target gene. LOC256021 BINDING SITE is HOST TARGET binding site found in the 3′ untranslated region of mRNA encoded by LOC256021, corresponding to a HOST TARGET binding site such as BINDING SITE I, BINDING SITE II or BINDING SITE III. Table 2 illustrates the complementarity of the nucleotide sequences of LOC256021 BINDING SITE, designated SEQ ID:3708, to the nucleotide sequence of VGAM362 RNA, herein designated VGAM RNA, also designated SEQ ID:697.

Another function of VGAM362 is therefore inhibition of LOC256021 (Accession XM_{—}172884). Accordingly, utilities of VGAM362 include diagnosis, prevention and treatment of diseases and clinical conditions associated with LOC256021. LOC91650 (Accession XM_{—}039853) is another VGAM362 host target gene. LOC91650 BINDING SITE is HOST TARGET binding site found in the 5′ untranslated region of mRNA encoded by LOC91650, corresponding to a HOST TARGET binding site such as BINDING SITE I, BINDING SITE II or BINDING SITE III. Table 2 illustrates the complementarity of the nucleotide sequences of LOC91650 BINDING SITE, designated SEQ ID:2765, to the nucleotide sequence of VGAM362 RNA, herein designated VGAM RNA, also designated SEQ ID:697.

Another function of VGAM362 is therefore inhibition of LOC91650 (Accession XM_{—}039853). Accordingly, utilities of VGAM362 include diagnosis, prevention and treatment of diseases and clinical conditions associated with LOC91650. LOC91752 (Accession XM_{—}040403) is another VGAM362 host target gene. LOC91752 BINDING SITE is HOST TARGET binding site found in the 5′ untranslated region of mRNA encoded by LOC91752, corresponding to a HOST TARGET binding site such as BINDING SITE I, BINDING SITE II or BINDING SITE III. Table 2 illustrates the complementarity of the nucleotide sequences of LOC91752 BINDING SITE, designated SEQ ID:2777, to the nucleotide sequence of VGAM362 RNA, herein designated VGAM RNA, also designated SEQ ID:697.

Another function of VGAM362 is therefore inhibition of LOC91752 (Accession XM_{—}040403). Accordingly, utilities of VGAM362 include diagnosis, prevention and treatment of diseases and clinical conditions associated with LOC91752. FIG. 1 further provides a conceptual description of a novel bioinformatically detected viral gene of the present invention, referred to here as Viral Genomic Address Messenger 363 (VGAM363) viral gene, which modulates expression of respective host target genes thereof, the function and utility of which host target genes is known in the art.

VGAM363 is a novel bioinformatically detected regulatory, non protein coding, viral micro RNA (miRNA) gene. The method by which VGAM363 was detected is described hereinabove with reference to FIGS. 1-8.

VGAM363 gene, herein designated VGAM GENE, is a viral gene contained in the genome of Vaccinia Virus. VGAM363 host target gene, herein designated VGAM HOST TARGET GENE, is a human gene contained in the human genome.

VGAM363 gene encodes a VGAM363 precursor RNA, herein designated VGAM PRECURSOR RNA. Similar to other miRNA genes, and unlike most ordinary genes, VGAM363 precursor RNA does not encode a protein. A nucleotide sequence identical or highly similar to the nucleotide sequence of VGAM363 precursor RNA is designated SEQ ID:349, and is provided hereinbelow with reference to the sequence listing part. Nucleotide sequence SEQ ID:349 is located at position 185510 relative to the genome of Vaccinia Virus.

VGAM363 precursor RNA folds onto itself, forming VGAM363 folded precursor RNA, herein designated VGAM FOLDED PRECURSOR RNA, which has a two-dimensional ‘hairpin structure’. As is well known in the art, this ‘hairpin structure’, is typical of RNA encoded by miRNA genes, and is due to the fact that the nucleotide sequence of the first half of the RNA encoded by a miRNA gene is an accurate or partial inversed-reversed sequence of the nucleotide sequence of the second half thereof.

An enzyme complex designated DICER COMPLEX, ‘dices’ the VGAM363 folded precursor RNA into VGAM363 RNA, herein designated VGAM RNA, a single stranded ˜22 nt long RNA segment. As is known in the art, dicing of a hairpin structured RNA precursor product into a short ˜22 nt RNA segment is catalyzed by an enzyme complex comprising an enzyme called Dicer together with other necessary proteins. A probable (over 87%) nucleotide sequence of VGAM363 RNA is designated SEQ ID:698, and is provided hereinbelow with reference to the sequence listing part.

VGAM363 host target gene, herein designated VGAM HOST TARGET GENE, encodes a corresponding messenger RNA, VGAM363 host target RNA, herein designated VGAM HOST TARGET RNA. VGAM363 host target RNA comprises three regions, as is typical of mRNA of a protein coding gene: a 5′ untranslated region, a protein coding region and a 3′ untranslated region, designated 5′UTR, PROTEIN CODING and 3′UTR respectively.

VGAM363 RNA, herein designated VGAM RNA, binds complementarily to one or more host target binding sites located in untranslated regions of VGAM363 host target RNA, herein designated VGAM HOST TARGET RNA. This complementary binding is due to the fact that the nucleotide sequence of VGAM363 RNA is an accurate or a partial inversed-reversed sequence of the nucleotide sequence of each of the host target binding sites. As an illustration, FIG. 1 shows three such host target binding sites, designated BINDING SITE I, BINDING SITE II and BINDING SITE III respectively. It is appreciated that the number of host target binding sites shown in FIG. 1 is meant as an illustration only, and is not meant to be limiting—VGAM363 RNA, herein designated VGAM RNA, may have a different number of host target binding sites in untranslated regions of a VGAM363 host target RNA, herein designated VGAM HOST TARGET RNA. It is further appreciated that while FIG. 1 depicts host target binding sites in the 3′UTR region, this is meant as an example only—these host target binding sites may be located in the 3′UTR region, the 5′UTR region, or in both 3′UTR and 5′UTR regions.

The complementary binding of VGAM363 RNA, herein designated VGAM RNA, to host target binding sites on VGAM363 host target RNA, herein designated VGAM HOST TARGET RNA, such as BINDING SITE I, BINDING SITE II and BINDING SITE III, inhibits translation of VGAM363 host target RNA into VGAM363 host target protein, herein designated VGAM HOST TARGET PROTEIN. VGAM host target protein is therefore outlined by a broken line.

It is appreciated that VGAM363 host target gene, herein designated VGAM HOST TARGET GENE, in fact represents a plurality of VGAM363 host target genes. The mRNA of each one of this plurality of VGAM363 host target genes comprises one or more host target binding sites, each having a nucleotide sequence which is at least partly complementary to VGAM363 RNA, herein designated VGAM RNA, and which when bound by VGAM363 RNA causes inhibition of translation of respective one or more VGAM363 host target proteins.

It is further appreciated by one skilled in the art that the mode of translational inhibition illustrated by FIG. 1 with specific reference to translational inhibition exerted by VGAM363 gene, herein designated VGAM GENE, on one or more VGAM363 host target gene, herein designated VGAM HOST TARGET GENE, is in fact common to other known non-viral miRNA genes. As mentioned hereinabove with reference to the background section, although a specific complementary binding site has been demonstrated only for some of the known miRNA genes (primarily Lin-4 and Let-7), all other recently discovered miRNA genes are also believed by those skilled in the art to modulate expression of other genes by complementary binding, although specific complementary binding sites of these other miRNA genes have not yet been found (Ruvkun G., ‘Perspective: Glimpses of a tiny RNA world’, Science 294, 779 (2001)).

It is yet further appreciated that a function of VGAM363 is inhibition of expression of host target genes, as part of a novel viral mechanism of attacking a host. Accordingly, utilities of VGAM363 include diagnosis, prevention and treatment of viral infection by Vaccinia Virus. Specific functions, and accordingly utilities, of VGAM363 correlate with, and may be deduced from, the identity of the host target genes which VGAM363 binds and inhibits, and the function of these host target genes, as elaborated hereinbelow.

Nucleotide sequences of the VGAM363 precursor RNA, herein designated VGAM PRECURSOR RNA, and of the ‘diced’ VGAM363 RNA, herein designated VGAM RNA, and a schematic representation of the secondary folding of VGAM363 folded precursor RNA, herein designated VGAM FOLDED PRECURSOR RNA, of VGAM363 are further described hereinbelow with reference to Table 1.

Nucleotide sequences of host target binding sites, such as BINDING SITE-I, BINDING SITE-II and BINDING SITE-III of FIG. 1, found on VGAM363 host target RNA, and schematic representation of the complementarity of each of these host target binding sites to VGAM363 RNA, herein designated VGAM RNA, are described hereinbelow with reference to Table 2.

As mentioned hereinabove with reference to FIG. 1, a function of VGAM363 gene, herein designated VGAM is inhibition of expression of VGAM363 target genes. It is appreciated that specific functions, and accordingly utilities, of VGAM363 correlate with, and may be deduced from, the identity of the target genes which VGAM363 binds and inhibits, and the function of these target genes, as elaborated hereinbelow.

Archain 1 (ARCN1, Accession NM_{—}001655) is a VGAM363 host target gene. ARCN1 BINDING SITE is HOST TARGET binding site found in the 3′ untranslated region of mRNA encoded by ARCN1, corresponding to a HOST TARGET binding site such as BINDING SITE I, BINDING SITE II or BINDING SITE III. Table 2 illustrates the complementarity of the nucleotide sequences of ARCN1 BINDING SITE, designated SEQ ID:844, to the nucleotide sequence of VGAM363 RNA, herein designated VGAM RNA, also designated SEQ ID:698.

A function of VGAM363 is therefore inhibition of Archain 1 (ARCN1, Accession NM_{—}001655), a gene which plays a fundamental role in eukaryotic cell biology. Accordingly, utilities of VGAM363 include diagnosis, prevention and treatment of diseases and clinical conditions associated with ARCN1. The function of ARCN1 and its association with various diseases and clinical conditions, has been established by previous studies, as described hereinabove with reference to VGAM55. Cathepsin K (pycnodysostosis) (CTSK, Accession NM_{—}000396) is another VGAM363 host target gene. CTSK BINDING SITE is HOST TARGET binding site found in the 3′ untranslated region of mRNA encoded by CTSK, corresponding to a HOST TARGET binding site such as BINDING SITE I, BINDING SITE II or BINDING SITE III. Table 2 illustrates the complementarity of the nucleotide sequences of CTSK BINDING SITE, designated SEQ ID:736, to the nucleotide sequence of VGAM363 RNA, herein designated VGAM RNA, also designated SEQ ID:698.

Another function of VGAM363 is therefore inhibition of Cathepsin K (pycnodysostosis) (CTSK, Accession NM_{—}000396). Accordingly, utilities of VGAM363 include diagnosis, prevention and treatment of diseases and clinical conditions associated with CTSK. High-mobility Group 20A (HMG20A, Accession NM_{—}018200) is another VGAM363 host target gene. HMG20A BINDING SITE is HOST TARGET binding site found in the 3′ untranslated region of mRNA encoded by HMG20A, corresponding to a HOST TARGET binding site such as BINDING SITE I, BINDING SITE II or BINDING SITE III. Table 2 illustrates the complementarity of the nucleotide sequences of HMG20A BINDING SITE, designated SEQ ID:1800, to the nucleotide sequence of VGAM363 RNA, herein designated VGAM RNA, also designated SEQ ID:698.

Another function of VGAM363 is therefore inhibition of High-mobility Group 20A (HMG20A, Accession NM_{—}018200). Accordingly, utilities of VGAM363 include diagnosis, prevention and treatment of diseases and clinical conditions associated with HMG20A. PR Domain Containing 2, with ZNF Domain (PRDM2, Accession NM_{—}012231) is another VGAM363 host target gene. PRDM2 BINDING SITE is HOST TARGET binding site found in the 3′ untranslated region of mRNA encoded by PRDM2, corresponding to a HOST TARGET binding site such as BINDING SITE I, BINDING SITE II or BINDING SITE III. Table 2 illustrates the complementarity of the nucleotide sequences of PRDM2 BINDING SITE, designated SEQ ID: 1415, to the nucleotide sequence of VGAM363 RNA, herein designated VGAM RNA, also designated SEQ ID:698.

Another function of VGAM363 is therefore inhibition of PR Domain Containing 2, with ZNF Domain (PRDM2, Accession NM_{—}012231), a gene which plays a role in transcriptional regulation during neuronal differentiation and pathogenesis of retinoblastoma. Accordingly, utilities of VGAM363 include diagnosis, prevention and treatment of diseases and clinical conditions associated with PRDM2. The function of PRDM2 and its association with various diseases and clinical conditions, has been established by previous studies, as described hereinabove with reference to VGAM55. RNA (guanine-7-) Methyltransferase (RNMT, Accession NM_{—}003799) is another VGAM363 host target gene. RNMT BINDING SITE is HOST TARGET binding site found in the 3′ untranslated region of mRNA encoded by RNMT, corresponding to a HOST TARGET binding site such as BINDING SITE I, BINDING SITE II or BINDING SITE III. Table 2 illustrates the complementarity of the nucleotide sequences of RNMT BINDING SITE, designated SEQ ID: 1060, to the nucleotide sequence of VGAM363 RNA, herein designated VGAM RNA, also designated SEQ ID:698.

Another function of VGAM363 is therefore inhibition of RNA (guanine-7-) Methyltransferase (RNMT, Accession NM_{—}003799), a gene which catalyzes the methylation of GpppN- at the guanine N7 position. Accordingly, utilities of VGAM363 include diagnosis, prevention and treatment of diseases and clinical conditions associated with RNMT. The function of RNMT and its association with various diseases and clinical conditions, has been established by previous studies, as described hereinabove with reference to VGAM55. Src Family Associated Phosphoprotein 1 (SCAP1, Accession NM_{—}003726) is another VGAM363 host target gene. SCAP1 BINDING SITE is HOST TARGET binding site found in the 3′ untranslated region of mRNA encoded by SCAP1, corresponding to a HOST TARGET binding site such as BINDING SITE I, BINDING SITE II or BINDING SITE III. Table 2 illustrates the complementarity of the nucleotide sequences of SCAP1 BINDING SITE, designated SEQ ID: 1052, to the nucleotide sequence of VGAM363 RNA, herein designated VGAM RNA, also designated SEQ ID:698.

Another function of VGAM363 is therefore inhibition of Src Family Associated Phosphoprotein 1 (SCAP1, Accession NM_{—}003726). Accordingly, utilities of VGAM363 include diagnosis, prevention and treatment of diseases and clinical conditions associated with SCAP1. Usher Syndrome 3A (USH3A, Accession NM_{—}052995) is another VGAM363 host target gene. USH3A BINDING SITE is HOST TARGET binding site found in the 5′ untranslated region of mRNA encoded by USH3A, corresponding to a HOST TARGET binding site such as BINDING SITE I, BINDING SITE II or BINDING SITE III. Table 2 illustrates the complementarity of the nucleotide sequences of USH3A BINDING SITE, designated SEQ ID:2355, to the nucleotide sequence of VGAM363 RNA, herein designated VGAM RNA, also designated SEQ ID:698.

Another function of VGAM363 is therefore inhibition of Usher Syndrome 3A (USH3A, Accession NM_{—}052995). Accordingly, utilities of VGAM363 include diagnosis, prevention and treatment of diseases and clinical conditions associated with USH3A. Vesicle Amine Transport Protein 1 Homolog (T californica) (VAT1, Accession NM_{—}006373) is another VGAM363 host target gene. VAT1 BINDING SITE is HOST TARGET binding site found in the 3′ untranslated region of mRNA encoded by VAT1, corresponding to a HOST TARGET binding site such as BINDING SITE I, BINDING SITE II or BINDING SITE III. Table 2 illustrates the complementarity of the nucleotide sequences of VAT1 BINDING SITE, designated SEQ ID:1292, to the nucleotide sequence of VGAM363 RNA, herein designated VGAM RNA, also designated SEQ ID:698.

Another function of VGAM363 is therefore inhibition of Vesicle Amine Transport Protein 1 Homolog (T californica) (VAT1, Accession NM_{—}006373), a gene which is a membrane protein of cholinergic synaptic vesicles. Accordingly, utilities of VGAM363 include diagnosis, prevention and treatment of diseases and clinical conditions associated with VAT1. The function of VAT1 and its association with various diseases and clinical conditions, has been established by previous studies, as described hereinabove with reference to VGAM55. Allograft Inflammatory Factor 1 (AIF1, Accession NM_{—}032955) is another VGAM363 host target gene. AIF1 BINDING SITE is HOST TARGET binding site found in the 5′ untranslated region of mRNA encoded by AIF1, corresponding to a HOST TARGET binding site such as BINDING SITE I, BINDING SITE II or BINDING SITE III. Table 2 illustrates the complementarity of the nucleotide sequences of AIF1 BINDING SITE, designated SEQ ID:2297, to the nucleotide sequence of VGAM363 RNA, herein designated VGAM RNA, also designated SEQ ID:698.

Another function of VGAM363 is therefore inhibition of Allograft Inflammatory Factor 1 (AIF1, Accession NM_{—}032955). Accordingly, utilities of VGAM363 include diagnosis, prevention and treatment of diseases and clinical conditions associated with AIF1. Complexin 1 (CPLX1, Accession NM_{—}006651) is another VGAM363 host target gene. CPLX1 BINDING SITE is HOST TARGET binding site found in the 3′ untranslated region of mRNA encoded by CPLX1, corresponding to a HOST TARGET binding site such as BINDING SITE I, BINDING SITE II or BINDING SITE III. Table 2 illustrates the complementarity of the nucleotide sequences of CPLX1 BINDING SITE, designated SEQ ID:1317, to the nucleotide sequence of VGAM363 RNA, herein designated VGAM RNA, also designated SEQ ID:698.

Another function of VGAM363 is therefore inhibition of Complexin 1 (CPLX1, Accession NM_{—}006651). Accordingly, utilities of VGAM363 include diagnosis, prevention and treatment of diseases and clinical conditions associated with CPLX1. CXYorf1 (Accession XM_{—}088704) is another VGAM363 host target gene. CXYorf1 BINDING SITE is HOST TARGET binding site found in the 3′ untranslated region of mRNA encoded by CXYorf1, corresponding to a HOST TARGET binding site such as BINDING SITE I, BINDING SITE II or BINDING SITE III. Table 2 illustrates the complementarity of the nucleotide sequences of CXYorf1 BINDING SITE, designated SEQ ID:3228, to the nucleotide sequence of VGAM363 RNA, herein designated VGAM RNA, also designated SEQ ID:698.

Another function of VGAM363 is therefore inhibition of CXYorf1 (Accession XM_{—}088704). Accordingly, utilities of VGAM363 include diagnosis, prevention and treatment of diseases and clinical conditions associated with CXYorf1. FLJ12221 (Accession XM_{—}031342) is another VGAM363 host target gene. FLJ12221 BINDING SITE is HOST TARGET binding site found in the 3′ untranslated region of mRNA encoded by FLJ12221, corresponding to a HOST TARGET binding site such as BINDING SITE I, BINDING SITE II or BINDING SITE III. Table 2 illustrates the complementarity of the nucleotide sequences of FLJ12221 BINDING SITE, designated SEQ ID:2637, to the nucleotide sequence of VGAM363 RNA, herein designated VGAM RNA, also designated SEQ ID:698.

Another function of VGAM363 is therefore inhibition of FLJ12221 (Accession XM_{—}031342). Accordingly, utilities of VGAM363 include diagnosis, prevention and treatment of diseases and clinical conditions associated with FLJ12221. FLJ20207 (Accession NM_{—}017711) is another VGAM363 host target gene. FLJ20207 BINDING SITE is HOST TARGET binding site found in the 3′ untranslated region of mRNA encoded by FLJ20207, corresponding to a HOST TARGET binding site such as BINDING SITE I, BINDING SITE II or BINDING SITE III. Table 2 illustrates the complementarity of the nucleotide sequences of FLJ20207 BINDING SITE, designated SEQ ID:1741, to the nucleotide sequence of VGAM363 RNA, herein designated VGAM RNA, also designated SEQ ID:698.

Another function of VGAM363 is therefore inhibition of FLJ20207 (Accession NM_{—}017711). Accordingly, utilities of VGAM363 include diagnosis, prevention and treatment of diseases and clinical conditions associated with FLJ20207. FLJ22215 (Accession XM_{—}173021) is another VGAM363 host target gene. FLJ22215 BINDING SITE is HOST TARGET binding site found in the 3′ untranslated region of mRNA encoded by FLJ22215, corresponding to a HOST TARGET binding site such as BINDING SITE I, BINDING SITE II or BINDING SITE III. Table 2 illustrates the complementarity of the nucleotide sequences of FLJ22215 BINDING SITE, designated SEQ ID:3715, to the nucleotide sequence of VGAM363 RNA, herein designated VGAM RNA, also designated SEQ ID:698.

Another function of VGAM363 is therefore inhibition of FLJ22215 (Accession XM_{—}173021). Accordingly, utilities of VGAM363 include diagnosis, prevention and treatment of diseases and clinical conditions associated with FLJ22215. FLJ31547 (Accession NM_{—}145024) is another VGAM363 host target gene. FLJ31547 BINDING SITE is HOST TARGET binding site found in the 3′ untranslated region of mRNA encoded by FLJ31547, corresponding to a HOST TARGET binding site such as BINDING SITE I, BINDING SITE II or BINDING SITE III. Table 2 illustrates the complementarity of the nucleotide sequences of FLJ31547 BINDING SITE, designated SEQ ID:2511, to the nucleotide sequence of VGAM363 RNA, herein designated VGAM RNA, also designated SEQ ID:698.

Another function of VGAM363 is therefore inhibition of FLJ31547 (Accession NM_{—}145024). Accordingly, utilities of VGAM363 include diagnosis, prevention and treatment of diseases and clinical conditions associated with FLJ31547. HSCBCIP1 (Accession XM_{—}114210) is another VGAM363 host target gene. HSCBCIP1 BINDING SITE is HOST TARGET binding site found in the 3′ untranslated region of mRNA encoded by HSCBCIP1, corresponding to a HOST TARGET binding site such as BINDING SITE I, BINDING SITE II or BINDING SITE III. Table 2 illustrates the complementarity of the nucleotide sequences of HSCBCIP1 BINDING SITE, designated SEQ ID:3449, to the nucleotide sequence of VGAM363 RNA, herein designated VGAM RNA, also designated SEQ ID:698.

Another function of VGAM363 is therefore inhibition of HSCBCIP1 (Accession XM_{—}114210). Accordingly, utilities of VGAM363 include diagnosis, prevention and treatment of diseases and clinical conditions associated with HSCBCIP1. KIAA0515 (Accession XM_{—}033380) is another VGAM363 host target gene. KIAA0515 BINDING SITE is HOST TARGET binding site found in the 5′ untranslated region of mRNA encoded by KIAA0515, corresponding to a HOST TARGET binding site such as BINDING SITE I, BINDING SITE II or BINDING SITE III. Table 2 illustrates the complementarity of the nucleotide sequences of KIAA0515 BINDING SITE, designated SEQ ID:2670, to the nucleotide sequence of VGAM363 RNA, herein designated VGAM RNA, also designated SEQ ID:698.

Another function of VGAM363 is therefore inhibition of KIAA0515 (Accession XM_{—}033380). Accordingly, utilities of VGAM363 include diagnosis, prevention and treatment of diseases and clinical conditions associated with KIAA0515. KIAA1514 (Accession NM_{—}019064) is another VGAM363 host target gene. KIAA1514 BINDING SITE is HOST TARGET binding site found in the 3′ untranslated region of mRNA encoded by KIAA1514, corresponding to a HOST TARGET binding site such as BINDING SITE I, BINDING SITE II or BINDING SITE III. Table 2 illustrates the complementarity of the nucleotide sequences of KIAA1514 BINDING SITE, designated SEQ ID:1877, to the nucleotide sequence of VGAM363 RNA, herein designated VGAM RNA, also designated SEQ ID:698.

Another function of VGAM363 is therefore inhibition of KIAA1514 (Accession NM_{—}019064). Accordingly, utilities of VGAM363 include diagnosis, prevention and treatment of diseases and clinical conditions associated with KIAA1514. KIAA1553 (Accession XM_{—}166320) is another VGAM363 host target gene. KIAA1553 BINDING SITE is HOST TARGET binding site found in the 3′ untranslated region of mRNA encoded by KIAA1553, corresponding to a HOST TARGET binding site such as BINDING SITE I, BINDING SITE II or BINDING SITE III. Table 2 illustrates the complementarity of the nucleotide sequences of KIAA1553 BINDING SITE, designated SEQ ID:3544, to the nucleotide sequence of VGAM363 RNA, herein designated VGAM RNA, also designated SEQ ID:698.

Another function of VGAM363 is therefore inhibition of KIAA1553 (Accession XM_{—}166320). Accordingly, utilities of VGAM363 include diagnosis, prevention and treatment of diseases and clinical conditions associated with KIAA1553. KIAA1656 (Accession XM_{—}038022) is another VGAM363 host target gene. KIAA1656 BINDING SITE is HOST TARGET binding site found in the 3′ untranslated region of mRNA encoded by KIAA1656, corresponding to a HOST TARGET binding site such as BINDING SITE I, BINDING SITE II or BINDING SITE III. Table 2 illustrates the complementarity of the nucleotide sequences of KIAA1656 BINDING SITE, designated SEQ ID:2730, to the nucleotide sequence of VGAM363 RNA, herein designated VGAM RNA, also designated SEQ ID:698.

Another function of VGAM363 is therefore inhibition of KIAA1656 (Accession XM_{—}038022). Accordingly, utilities of VGAM363 include diagnosis, prevention and treatment of diseases and clinical conditions associated with KIAA1656. KIAA1877 (Accession XM_{—}038616) is another VGAM363 host target gene. KIAA1877 BINDING SITE is HOST TARGET binding site found in the 3′ untranslated region of mRNA encoded by KIAA1877, corresponding to a HOST TARGET binding site such as BINDING SITE I, BINDING SITE II or BINDING SITE III. Table 2 illustrates the complementarity of the nucleotide sequences of KIAA1877 BINDING SITE, designated SEQ ID:2741, to the nucleotide sequence of VGAM363 RNA, herein designated VGAM RNA, also designated SEQ ID:698.

Another function of VGAM363 is therefore inhibition of KIAA1877 (Accession XM_{—}038616). Accordingly, utilities of VGAM363 include diagnosis, prevention and treatment of diseases and clinical conditions associated with KIAA1877. MTCH1 (Accession NM_{—}014341) is another VGAM363 host target gene. MTCH1 BINDING SITE is HOST TARGET binding site found in the 3′ untranslated region of mRNA encoded by MTCH1, corresponding to a HOST TARGET binding site such as BINDING SITE I, BINDING SITE II or BINDING SITE III. Table 2 illustrates the complementarity of the nucleotide sequences of MTCH1 BINDING SITE, designated SEQ ID: 1493, to the nucleotide sequence of VGAM363 RNA, herein designated VGAM RNA, also designated SEQ ID:698.

Another function of VGAM363 is therefore inhibition of MTCH1 (Accession NM_{—}014341). Accordingly, utilities of VGAM363 include diagnosis, prevention and treatment of diseases and clinical conditions associated with MTCH1. N-PAC (Accession XM_{—}048113) is another VGAM363 host target gene. N-PAC BINDING SITE is HOST TARGET binding site found in the 3′ untranslated region of mRNA encoded by N-PAC, corresponding to a HOST TARGET binding site such as BINDING SITE I, BINDING SITE II or BINDING SITE III. Table 2 illustrates the complementarity of the nucleotide sequences of N-PAC BINDING SITE, designated SEQ ID:2902, to the nucleotide sequence of VGAM363 RNA, herein designated VGAM RNA, also designated SEQ ID:698.

Another function of VGAM363 is therefore inhibition of N-PAC (Accession XM_{—}048113). Accordingly, utilities of VGAM363 include diagnosis, prevention and treatment of diseases and clinical conditions associated with N-PAC. Sema Domain, Immunoglobulin Domain (Ig), Transmembrane Domain (TM) and Short Cytoplasmic Domain, (semaphorin) 4B (SEMA4B, Accession XM_{—}044533) is another VGAM363 host target gene. SEMA4B BINDING SITE is HOST TARGET binding site found in the 5′ untranslated region of mRNA encoded by SEMA4B, corresponding to a HOST TARGET binding site such as BINDING SITE I, BINDING SITE II or BINDING SITE III. Table 2 illustrates the complementarity of the nucleotide sequences of SEMA4B BINDING SITE, designated SEQ ID:2839, to the nucleotide sequence of VGAM363 RNA, herein designated VGAM RNA, also designated SEQ ID:698.

Another function of VGAM363 is therefore inhibition of Sema Domain, Immunoglobulin Domain (Ig), Transmembrane Domain (TM) and Short Cytoplasmic Domain, (semaphorin) 4B (SEMA4B, Accession XM_{—}044533). Accordingly, utilities of VGAM363 include diagnosis, prevention and treatment of diseases and clinical conditions associated with SEMA4B. Sprouty Homolog 1, Antagonist of FGF Signaling (Drosophila) (SPRY1, Accession XM_{—}036349) is another VGAM363 host target gene. SPRY1 BINDING SITE is HOST TARGET binding site found in the 3′ untranslated region of mRNA encoded by SPRY1, corresponding to a HOST TARGET binding site such as BINDING SITE I, BINDING SITE II or BINDING SITE III. Table 2 illustrates the complementarity of the nucleotide sequences of SPRY1 BINDING SITE, designated SEQ ID:2709, to the nucleotide sequence of VGAM363 RNA, herein designated VGAM RNA, also designated SEQ ID:698.

Another function of VGAM363 is therefore inhibition of Sprouty Homolog 1, Antagonist of FGF Signaling (Drosophila) (SPRY1, Accession XM_{—}036349). Accordingly, utilities of VGAM363 include diagnosis, prevention and treatment of diseases and clinical conditions associated with SPRY1. WS-3 (Accession NM_{—}006571) is another VGAM363 host target gene. WS-3 BINDING SITE is HOST TARGET binding site found in the 5′ untranslated region of mRNA encoded by WS-3, corresponding to a HOST TARGET binding site such as BINDING SITE I, BINDING SITE II or BINDING SITE III. Table 2 illustrates the complementarity of the nucleotide sequences of WS-3 BINDING SITE, designated SEQ ID:1312, to the nucleotide sequence of VGAM363 RNA, herein designated VGAM RNA, also designated SEQ ID:698.

Another function of VGAM363 is therefore inhibition of WS-3 (Accession NM_{—}006571). Accordingly, utilities of VGAM363 include diagnosis, prevention and treatment of diseases and clinical conditions associated with WS-3. LOC123242 (Accession XM_{—}063548) is another VGAM363 host target gene. LOC123242 BINDING SITE is HOST TARGET binding site found in the 5′ untranslated region of mRNA encoded by LOC123242, corresponding to a HOST TARGET binding site such as BINDING SITE I, BINDING SITE II or BINDING SITE III. Table 2 illustrates the complementarity of the nucleotide sequences of LOC123242 BINDING SITE, designated SEQ ID:3030, to the nucleotide sequence of VGAM363 RNA, herein designated VGAM RNA, also designated SEQ ID:698.

Another function of VGAM363 is therefore inhibition of LOC123242 (Accession XM_{—}063548). Accordingly, utilities of VGAM363 include diagnosis, prevention and treatment of diseases and clinical conditions associated with LOC123242. LOC127702 (Accession XM_{—}060619) is another VGAM363 host target gene. LOC127702 BINDING SITE is HOST TARGET binding site found in the 3′ untranslated region of mRNA encoded by LOC127702, corresponding to a HOST TARGET binding site such as BINDING SITE I, BINDING SITE II or BINDING SITE III. Table 2 illustrates the complementarity of the nucleotide sequences of LOC127702 BINDING SITE, designated SEQ ID:3024, to the nucleotide sequence of VGAM363 RNA, herein designated VGAM RNA, also designated SEQ ID:698.

Another function of VGAM363 is therefore inhibition of LOC127702 (Accession XM_{—}060619). Accordingly, utilities of VGAM363 include diagnosis, prevention and treatment of diseases and clinical conditions associated with LOC127702. LOC146745 (Accession XM_{—}085577) is another VGAM363 host target gene. LOC146745 BINDING SITE is HOST TARGET binding site found in the 5′ untranslated region of mRNA encoded by LOC146745, corresponding to a HOST TARGET binding site such as BINDING SITE I, BINDING SITE II or BINDING SITE III. Table 2 illustrates the complementarity of the nucleotide sequences of LOC146745 BINDING SITE, designated SEQ ID:3099, to the nucleotide sequence of VGAM363 RNA, herein designated VGAM RNA, also designated SEQ ID:698.

Another function of VGAM363 is therefore inhibition of LOC146745 (Accession XM_{—}085577). Accordingly, utilities of VGAM363 include diagnosis, prevention and treatment of diseases and clinical conditions associated with LOC146745. LOC147791 (Accession XM_{—}097293) is another VGAM363 host target gene. LOC147791 BINDING SITE is HOST TARGET binding site found in the 3′ untranslated region of mRNA encoded by LOC147791, corresponding to a HOST TARGET binding site such as BINDING SITE I, BINDING SITE II or BINDING SITE III. Table 2 illustrates the complementarity of the nucleotide sequences of LOC147791 BINDING SITE, designated SEQ ID:3294, to the nucleotide sequence of VGAM363 RNA, herein designated VGAM RNA, also designated SEQ ID:698.

Another function of VGAM363 is therefore inhibition of LOC147791 (Accession XM_{—}097293). Accordingly, utilities of VGAM363 include diagnosis, prevention and treatment of diseases and clinical conditions associated with LOC147791. LOC196892 (Accession XM_{—}113768) is another VGAM363 host target gene. LOC196892 BINDING SITE is HOST TARGET binding site found in the 3′ untranslated region of mRNA encoded by LOC196892, corresponding to a HOST TARGET binding site such as BINDING SITE I, BINDING SITE II or BINDING SITE III. Table 2 illustrates the complementarity of the nucleotide sequences of LOC196892 BINDING SITE, designated SEQ ID:3417, to the nucleotide sequence of VGAM363 RNA, herein designated VGAM RNA, also designated SEQ ID:698.

Another function of VGAM363 is therefore inhibition of LOC196892 (Accession XM_{—}113768). Accordingly, utilities of VGAM363 include diagnosis, prevention and treatment of diseases and clinical conditions associated with LOC196892. LOC200093 (Accession XM_{—}032184) is another VGAM363 host target gene. LOC200093 BINDING SITE is HOST TARGET binding site found in the 3′ untranslated region of mRNA encoded by LOC200093, corresponding to a HOST TARGET binding site such as BINDING SITE I, BINDING SITE II or BINDING SITE III. Table 2 illustrates the complementarity of the nucleotide sequences of LOC200093 BINDING SITE, designated SEQ ID:2648, to the nucleotide sequence of VGAM363 RNA, herein designated VGAM RNA, also designated SEQ ID:698.

Another function of VGAM363 is therefore inhibition of LOC200093 (Accession XM_{—}032184). Accordingly, utilities of VGAM363 include diagnosis, prevention and treatment of diseases and clinical conditions associated with LOC200093. LOC201243 (Accession XM_{—}113935) is another VGAM363 host target gene. LOC201243 BINDING SITE is HOST TARGET binding site found in the 5′ untranslated region of mRNA encoded by LOC201243, corresponding to a HOST TARGET binding site such as BINDING SITE I, BINDING SITE II or BINDING SITE III. Table 2 illustrates the complementarity of the nucleotide sequences of LOC201243 BINDING SITE, designated SEQ ID:3426, to the nucleotide sequence of VGAM363 RNA, herein designated VGAM RNA, also designated SEQ ID:698.

Another function of VGAM363 is therefore inhibition of LOC201243 (Accession XM_{—}113935). Accordingly, utilities of VGAM363 include diagnosis, prevention and treatment of diseases and clinical conditions associated with LOC201243. LOC219404 (Accession XM_{—}167909) is another VGAM363 host target gene. LOC219404 BINDING SITE is HOST TARGET binding site found in the 3′ untranslated region of mRNA encoded by LOC219404, corresponding to a HOST TARGET binding site such as BINDING SITE I, BINDING SITE II or BINDING SITE III. Table 2 illustrates the complementarity of the nucleotide sequences of LOC219404 BINDING SITE, designated SEQ ID:3606, to the nucleotide sequence of VGAM363 RNA, herein designated VGAM RNA, also designated SEQ ID:698.

Another function of VGAM363 is therefore inhibition of LOC219404 (Accession XM_{—}167909). Accordingly, utilities of VGAM363 include diagnosis, prevention and treatment of diseases and clinical conditions associated with LOC219404. LOC222066 (Accession XM_{—}166582) is another VGAM363 host target gene. LOC222066 BINDING SITE is HOST TARGET binding site found in the 3′ untranslated region of mRNA encoded by LOC222066, corresponding to a HOST TARGET binding site such as BINDING SITE I, BINDING SITE II or BINDING SITE III. Table 2 illustrates the complementarity of the nucleotide sequences of LOC222066 BINDING SITE, designated SEQ ID:3574, to the nucleotide sequence of VGAM363 RNA, herein designated VGAM RNA, also designated SEQ ID:698.

Another function of VGAM363 is therefore inhibition of LOC222066 (Accession XM_{—}166582). Accordingly, utilities of VGAM363 include diagnosis, prevention and treatment of diseases and clinical conditions associated with LOC222066. LOC253001 (Accession XM_{—}171711) is another VGAM363 host target gene. LOC253001 BINDING SITE is HOST TARGET binding site found in the 5′ untranslated region of mRNA encoded by LOC253001, corresponding to a HOST TARGET binding site such as BINDING SITE I, BINDING SITE II or BINDING SITE III. Table 2 illustrates the complementarity of the nucleotide sequences of LOC253001 BINDING SITE, designated SEQ ID:3698, to the nucleotide sequence of VGAM363 RNA, herein designated VGAM RNA, also designated SEQ ID:698.

Another function of VGAM363 is therefore inhibition of LOC253001 (Accession XM_{—}171711). Accordingly, utilities of VGAM363 include diagnosis, prevention and treatment of diseases and clinical conditions associated with LOC253001. LOC91040 (Accession XM_{—}035641) is another VGAM363 host target gene. LOC91040 BINDING SITE is HOST TARGET binding site found in the 3′ untranslated region of mRNA encoded by LOC91040, corresponding to a HOST TARGET binding site such as BINDING SITE I, BINDING SITE II or BINDING SITE III. Table 2 illustrates the complementarity of the nucleotide sequences of LOC91040 BINDING SITE, designated SEQ ID:2700, to the nucleotide sequence of VGAM363 RNA, herein designated VGAM RNA, also designated SEQ ID:698.

Another function of VGAM363 is therefore inhibition of LOC91040 (Accession XM_{—}035641). Accordingly, utilities of VGAM363 include diagnosis, prevention and treatment of diseases and clinical conditions associated with LOC91040. FIG. 9 further provides a conceptual description of novel bioinformatically detected regulatory viral gene, referred to here as Viral Genomic Record 364(VGR364) viral gene, which encodes an ‘operon-like’ cluster of novel viral micro RNA-like genes, each of which in turn modulates expression of at least one host target gene, the function and utility of which at least one host target gene is known in the art.

VGR364 gene, herein designated VGR GENE, is a novel bioinformatically detected regulatory, non protein coding, RNA viral gene. The method by which VGR364 gene was detected is described hereinabove with reference to FIGS. 1-9.

VGR364 gene encodes VGR364 precursor RNA, herein designated VGR PRECURSOR RNA, an RNA molecule, typically several hundred nucleotides long.

VGR364 precursor RNA folds spatially, forming VGR364 folded precursor RNA, herein designated VGR FOLDED PRECURSOR RNA. It is appreciated that VGR364 folded precursor RNA comprises a plurality of what is known in the art as ‘hairpin’ structures. These ‘hairpin’ structures are due to the fact that the nucleotide sequence of VGR364 precursor RNA comprises a plurality of segments, the first half of each such segment having a nucleotide sequence which is at least a partial inversed-reversed sequence of the second half thereof, as is well known in the art.

VGR364 folded precursor RNA is naturally processed by cellular enzymatic activity into at least 8 separate VGAM precursor RNAs, VGAM15 precursor RNA, VGAM16 precursor RNA, VGAM17 precursor RNA, VGAM18 precursor RNA, VGAM19 precursor RNA, VGAM20 precursor RNA, VGAM21 precursor RNA and VGAM22 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM15 RNA, VGAM16 RNA, VGAM17 RNA, VGAM18 RNA, VGAM19 RNA, VGAM20 RNA, VGAM21 RNA and VGAM22 RNA, herein schematically represented by VGAM1 RNA through VGAM3 RNA, each of which VGAM RNAs corresponding to VGAM RNA of FIG. 1.

VGAM15 RNA, herein schematically represented by VGAM1 RNA through VGAM3 RNA, binds complementarily to a host target binding site located in an untranslated region of VGAM15 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM15 host target RNA into VGAM15 host target protein, herein schematically represented by VGAM1 HOST TARGET PROTEIN through VGAM3 HOST TARGET PROTEIN, all of FIG. 1.

VGAM16 RNA, herein schematically represented by VGAM1 RNA through VGAM3 RNA, binds complementarily to a host target binding site located in an untranslated region of VGAM16 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM16 host target RNA into VGAM16 host target protein, herein schematically represented by VGAM1 HOST TARGET PROTEIN through VGAM3 HOST TARGET PROTEIN, all of FIG. 1.

VGAM17 RNA, herein schematically represented by VGAM1 RNA through VGAM3 RNA, binds complementarily to a host target binding site located in an untranslated region of VGAM17 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM17 host target RNA into VGAM17 host target protein, herein schematically represented by VGAM1 HOST TARGET PROTEIN through VGAM3 HOST TARGET PROTEIN, all of FIG. 1.

VGAM18 RNA, herein schematically represented by VGAM1 RNA through VGAM3 RNA, binds complementarily to a host target binding site located in an untranslated region of VGAM18 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM18 host target RNA into VGAM18 host target protein, herein schematically represented by VGAM1 HOST TARGET PROTEIN through VGAM3 HOST TARGET PROTEIN, all of FIG. 1.

VGAM19 RNA, herein schematically represented by VGAM1 RNA through VGAM3 RNA, binds complementarily to a host target binding site located in an untranslated region of VGAM19 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM19 host target RNA into VGAM19 host target protein, herein schematically represented by VGAM1 HOST TARGET PROTEIN through VGAM3 HOST TARGET PROTEIN, all of FIG. 1.

VGAM20 RNA, herein schematically represented by VGAM1 RNA through VGAM3 RNA, binds complementarily to a host target binding site located in an untranslated region of VGAM20 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM20 host target RNA into VGAM20 host target protein, herein schematically represented by VGAM1 HOST TARGET PROTEIN through VGAM3 HOST TARGET PROTEIN, all of FIG. 1.

VGAM21 RNA, herein schematically represented by VGAM1 RNA through VGAM3 RNA, binds complementarily to a host target binding site located in an untranslated region of VGAM21 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM21 host target RNA into VGAM21 host target protein, herein schematically represented by VGAM1 HOST TARGET PROTEIN through VGAM3 HOST TARGET PROTEIN, all of FIG. 1.

VGAM22 RNA, herein schematically represented by VGAM1 RNA through VGAM3 RNA, binds complementarily to a host target binding site located in an untranslated region of VGAM22 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM22 host target RNA into VGAM22 host target protein, herein schematically represented by VGAM1 HOST TARGET PROTEIN through VGAM3 HOST TARGET PROTEIN, all of FIG. 1.

It is appreciated that a function of VGR364 gene, herein designated VGR GENE, is inhibition of expression of host target genes, as part of a novel viral mechanism of attacking a host. Accordingly, utilities of VGR364 gene include diagnosis, prevention and treatment of viral infection by Vaccinia Virus. Specific functions, and accordingly utilities, of VGR364 gene correlate with, and may be deduced from, the identity of the host target genes, which are inhibited by VGAM RNAs comprised in the ‘operon-like’ cluster of VGR364 gene: VGAM15 host target protein, VGAM16 host target protein, VGAM17 host target protein, VGAM18 host target protein, VGAM19 host target protein, VGAM20 host target protein, VGAM21 host target protein and VGAM22 host target protein, herein schematically represented by VGAM1 HOST TARGET PROTEIN through VGAM3 HOST TARGET PROTEIN. The function of these host target genes is elaborated hereinabove with reference to VGAM15, VGAM16, VGAM17, VGAM18, VGAM19, VGAM20, VGAM21 and VGAM22. FIG. 9 further provides a conceptual description of novel bioinformatically detected regulatory viral gene, referred to here as Viral Genomic Record 365(VGR365) viral gene, which encodes an ‘operon-like’ cluster of novel viral micro RNA-like genes, each of which in turn modulates expression of at least one host target gene, the function and utility of which at least one host target gene is known in the art.

VGR365 gene, herein designated VGR GENE, is a novel bioinformatically detected regulatory, non protein coding, RNA viral gene. The method by which VGR365 gene was detected is described hereinabove with reference to FIGS. 1-9.

VGR365 gene encodes VGR365 precursor RNA, herein designated VGR PRECURSOR RNA, an RNA molecule, typically several hundred nucleotides long.

VGR365 precursor RNA folds spatially, forming VGR365 folded precursor RNA, herein designated VGR FOLDED PRECURSOR RNA. It is appreciated that VGR365 folded precursor RNA comprises a plurality of what is known in the art as ‘hairpin’ structures. These ‘hairpin’ structures are due to the fact that the nucleotide sequence of VGR365 precursor RNA comprises a plurality of segments, the first half of each such segment having a nucleotide sequence which is at least a partial inversed-reversed sequence of the second half thereof, as is well known in the art.

VGR365 folded precursor RNA is naturally processed by cellular enzymatic activity into at least 8 separate VGAM precursor RNAs, VGAM23 precursor RNA, VGAM24 precursor RNA, VGAM25 precursor RNA, VGAM26 precursor RNA, VGAM27 precursor RNA, VGAM28 precursor RNA, VGAM29 precursor RNA and VGAM30 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM23 RNA, VGAM24 RNA, VGAM25 RNA, VGAM26 RNA, VGAM27 RNA, VGAM28 RNA, VGAM29 RNA and VGAM30 RNA, herein schematically represented by VGAM1 RNA through VGAM3 RNA, each of which VGAM RNAs corresponding to VGAM RNA of FIG. 1.

VGAM23 RNA, herein schematically represented by VGAM1 RNA through VGAM3 RNA, binds complementarily to a host target binding site located in an untranslated region of VGAM23 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM23 host target RNA into VGAM23 host target protein, herein schematically represented by VGAM1 HOST TARGET PROTEIN through VGAM3 HOST TARGET PROTEIN, all of FIG. 1.

VGAM24 RNA, herein schematically represented by VGAM1 RNA through VGAM3 RNA, binds complementarily to a host target binding site located in an untranslated region of VGAM24 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM24 host target RNA into VGAM24 host target protein, herein schematically represented by VGAM1 HOST TARGET PROTEIN through VGAM3 HOST TARGET PROTEIN, all of FIG. 1.

VGAM25 RNA, herein schematically represented by VGAM1 RNA through VGAM3 RNA, binds complementarily to a host target binding site located in an untranslated region of VGAM25 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM25 host target RNA into VGAM25 host target protein, herein schematically represented by VGAM1 HOST TARGET PROTEIN through VGAM3 HOST TARGET PROTEIN, all of FIG. 1.

VGAM26 RNA, herein schematically represented by VGAM1 RNA through VGAM3 RNA, binds complementarily to a host target binding site located in an untranslated region of VGAM26 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM26 host target RNA into VGAM26 host target protein, herein schematically represented by VGAM1 HOST TARGET PROTEIN through VGAM3 HOST TARGET PROTEIN, all of FIG. 1.

VGAM27 RNA, herein schematically represented by VGAM1 RNA through VGAM3 RNA, binds complementarily to a host target binding site located in an untranslated region of VGAM27 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM27 host target RNA into VGAM27 host target protein, herein schematically represented by VGAM1 HOST TARGET PROTEIN through VGAM3 HOST TARGET PROTEIN, all of FIG. 1.

VGAM28 RNA, herein schematically represented by VGAM1 RNA through VGAM3 RNA, binds complementarily to a host target binding site located in an untranslated region of VGAM28 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of 0FIG. 1, thereby inhibiting translation of VGAM28 host target RNA into VGAM28 host target protein, herein schematically represented by VGAM1 HOST TARGET PROTEIN through VGAM3 HOST TARGET PROTEIN, all of FIG. 1.

VGAM29 RNA, herein schematically represented by VGAM1 RNA through VGAM3 RNA, binds complementarily to a host target binding site located in an untranslated region of VGAM29 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM29 host target RNA into VGAM29 host target protein, herein schematically represented by VGAM1 HOST TARGET PROTEIN through VGAM3 HOST TARGET PROTEIN, all of FIG. 1.

VGAM30 RNA, herein schematically represented by VGAM1 RNA through VGAM3 RNA, binds complementarily to a host target binding site located in an untranslated region of VGAM30 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM30 host target RNA into VGAM30 host target protein, herein schematically represented by VGAM1 HOST TARGET PROTEIN through VGAM3 HOST TARGET PROTEIN, all of FIG. 1.

It is appreciated that a function of VGR365 gene, herein designated VGR GENE, is inhibition of expression of host target genes, as part of a novel viral mechanism of attacking a host. Accordingly, utilities of VGR365 gene include diagnosis, prevention and treatment of viral infection by Vaccinia Virus. Specific functions, and accordingly utilities, of VGR365 gene correlate with, and may be deduced from, the identity of the host target genes, which are inhibited by VGAM RNAs comprised in the ‘operon-like’ cluster of VGR365 gene: VGAM23 host target protein, VGAM24 host target protein, VGAM25 host target protein, VGAM26 host target protein, VGAM27 host target protein, VGAM28 host target protein, VGAM29 host target protein and VGAM30 host target protein, herein schematically represented by VGAM1 HOST TARGET PROTEIN through VGAM3 HOST TARGET PROTEIN. The function of these host target genes is elaborated hereinabove with reference to VGAM23, VGAM24, VGAM25, VGAM26, VGAM27, VGAM28, VGAM29 and VGAM30. FIG. 9 further provides a conceptual description of novel bioinformatically detected regulatory viral gene, referred to here as Viral Genomic Record 366(VGR366) viral gene, which encodes an ‘operon-like’ cluster of novel viral micro RNA-like genes, each of which in turn modulates expression of at least one host target gene, the function and utility of which at least one host target gene is known in the art.

VGR366 gene, herein designated VGR GENE, is a novel bioinformatically detected regulatory, non protein coding, RNA viral gene. The method by which VGR366 gene was detected is described hereinabove with reference to FIGS. 1-9.

VGR366 gene encodes VGR366 precursor RNA, herein designated VGR PRECURSOR RNA, an RNA molecule, typically several hundred nucleotides long.

VGR366 precursor RNA folds spatially, forming VGR366 folded precursor RNA, herein designated VGR FOLDED PRECURSOR RNA. It is appreciated that VGR366 folded precursor RNA comprises a plurality of what is known in the art as ‘hairpin’ structures. These ‘hairpin’ structures are due to the fact that the nucleotide sequence of VGR366 precursor RNA comprises a plurality of segments, the first half of each such segment having a nucleotide sequence which is at least a partial inversed-reversed sequence of the second half thereof, as is well known in the art.

VGR366 folded precursor RNA is naturally processed by cellular enzymatic activity into at least 4 separate VGAM precursor RNAs, VGAM31 precursor RNA, VGAM32 precursor RNA, VGAM33 precursor RNA and VGAM34 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM31 RNA, VGAM32 RNA, VGAM33 RNA and VGAM34 RNA, herein schematically represented by VGAM1 RNA through VGAM3 RNA, each of which VGAM RNAs corresponding to VGAM RNA of FIG. 1.

VGAM31 RNA, herein schematically represented by VGAM1 RNA through VGAM3 RNA, binds complementarily to a host target binding site located in an untranslated region of VGAM31 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM31 host target RNA into VGAM31 host target protein, herein schematically represented by VGAM1 HOST TARGET PROTEIN through VGAM3 HOST TARGET PROTEIN, all of FIG. 1.

VGAM32 RNA, herein schematically represented by VGAM1 RNA through VGAM3 RNA, binds complementarily to a host target binding site located in an untranslated region of VGAM32 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM32 host target RNA into VGAM32 host target protein, herein schematically represented by VGAM1 HOST TARGET PROTEIN through VGAM3 HOST TARGET PROTEIN, all of FIG. 1.

VGAM33 RNA, herein schematically represented by VGAM1 RNA through VGAM3 RNA, binds complementarily to a host target binding site located in an untranslated region of VGAM33 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM33 host target RNA into VGAM33 host target protein, herein schematically represented by VGAM1 HOST TARGET PROTEIN through VGAM3 HOST TARGET PROTEIN, all of FIG. 1.

VGAM34 RNA, herein schematically represented by VGAM1 RNA through VGAM3 RNA, binds complementarily to a host target binding site located in an untranslated region of VGAM34 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM34 host target RNA into VGAM34 host target protein, herein schematically represented by VGAM1 HOST TARGET PROTEIN through VGAM3 HOST TARGET PROTEIN, all of FIG. 1.

It is appreciated that a function of VGR366 gene, herein designated VGR GENE, is inhibition of expression of host target genes, as part of a novel viral mechanism of attacking a host. Accordingly, utilities of VGR366 gene include diagnosis, prevention and treatment of viral infection by Vaccinia Virus. Specific functions, and accordingly utilities, of VGR366 gene correlate with, and may be deduced from, the identity of the host target genes, which are inhibited by VGAM RNAs comprised in the ‘operon-like’ cluster of VGR366 gene: VGAM31 host target protein, VGAM32 host target protein, VGAM33 host target protein and VGAM34 host target protein, herein schematically represented by VGAM1 HOST TARGET PROTEIN through VGAM3 HOST TARGET PROTEIN. The function of these host target genes is elaborated hereinabove with reference to VGAM31, VGAM32, VGAM33 and VGAM34. FIG. 9 further provides a conceptual description of novel bioinformatically detected regulatory viral gene, referred to here as Viral Genomic Record 367(VGR367) viral gene, which encodes an ‘operon-like’ cluster of novel viral micro RNA-like genes, each of which in turn modulates expression of at least one host target gene, the function and utility of which at least one host target gene is known in the art.

VGR367 gene, herein designated VGR GENE, is a novel bioinformatically detected regulatory, non protein coding, RNA viral gene. The method by which VGR367 gene was detected is described hereinabove with reference to FIGS. 1-9.

VGR367 gene encodes VGR367 precursor RNA, herein designated VGR PRECURSOR RNA, an RNA molecule, typically several hundred nucleotides long.

VGR367 precursor RNA folds spatially, forming VGR367 folded precursor RNA, herein designated VGR FOLDED PRECURSOR RNA. It is appreciated that VGR367 folded precursor RNA comprises a plurality of what is known in the art as ‘hairpin’ structures. These ‘hairpin’ structures are due to the fact that the nucleotide sequence of VGR367 precursor RNA comprises a plurality of segments, the first half of each such segment having a nucleotide sequence which is at least a partial inversed-reversed sequence of the second half thereof, as is well known in the art.

VGR367 folded precursor RNA is naturally processed by cellular enzymatic activity into at least 8 separate VGAM precursor RNAs, VGAM35 precursor RNA, VGAM36 precursor RNA, VGAM37 precursor RNA, VGAM38 precursor RNA, VGAM39 precursor RNA, VGAM40 precursor RNA, VGAM41 precursor RNA and VGAM42 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM35 RNA, VGAM36 RNA, VGAM37 RNA, VGAM38 RNA, VGAM39 RNA, VGAM40 RNA, VGAM41 RNA and VGAM42 RNA, herein schematically represented by VGAM1 RNA through VGAM3 RNA, each of which VGAM RNAs corresponding to VGAM RNA of FIG. 1.

VGAM35 RNA, herein schematically represented by VGAM1 RNA through VGAM3 RNA, binds complementarily to a host target binding site located in an untranslated region of VGAM35 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM35 host target RNA into VGAM35 host target protein, herein schematically represented by VGAM1 HOST TARGET PROTEIN through VGAM3 HOST TARGET PROTEIN, all of FIG. 1.

VGAM36 RNA, herein schematically represented by VGAM1 RNA through VGAM3 RNA, binds complementarily to a host target binding site located in an untranslated region of VGAM36 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM36 host target RNA into VGAM36 host target protein, herein schematically represented by VGAM1 HOST TARGET PROTEIN through VGAM3 HOST TARGET PROTEIN, all of FIG. 1.

VGAM37 RNA, herein schematically represented by VGAM1 RNA through VGAM3 RNA, binds complementarily to a host target binding site located in an untranslated region of VGAM37 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM37 host target RNA into VGAM37 host target protein, herein schematically represented by VGAM1 HOST TARGET PROTEIN through VGAM3 HOST TARGET PROTEIN, all of FIG. 1.

VGAM38 RNA, herein schematically represented by VGAM1 RNA through VGAM3 RNA, binds complementarily to a host target binding site located in an untranslated region of VGAM38 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM38 host target RNA into VGAM38 host target protein, herein schematically represented by VGAM1 HOST TARGET PROTEIN through VGAM3 HOST TARGET PROTEIN, all of FIG. 1.

VGAM39 RNA, herein schematically represented by VGAM1 RNA through VGAM3 RNA, binds complementarily to a host target binding site located in an untranslated region of VGAM39 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM39 host target RNA into VGAM39 host target protein, herein schematically represented by VGAM1 HOST TARGET PROTEIN through VGAM3 HOST TARGET PROTEIN, all of FIG. 1.

VGAM40 RNA, herein schematically represented by VGAM1 RNA through VGAM3 RNA, binds complementarily to a host target binding site located in an untranslated region of VGAM40 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM40 host target RNA into VGAM40 host target protein, herein schematically represented by VGAM1 HOST TARGET PROTEIN through VGAM3 HOST TARGET PROTEIN, all of FIG. 1.

VGAM41 RNA, herein schematically represented by VGAM1 RNA through VGAM3 RNA, binds complementarily to a host target binding site located in an untranslated region of VGAM41 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM41 host target RNA into VGAM41 host target protein, herein schematically represented by VGAM1 HOST TARGET PROTEIN through VGAM3 HOST TARGET PROTEIN, all of FIG. 1.

VGAM42 RNA, herein schematically represented by VGAM1 RNA through VGAM3 RNA, binds complementarily to a host target binding site located in an untranslated region of VGAM42 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM42 host target RNA into VGAM42 host target protein, herein schematically represented by VGAM1 HOST TARGET PROTEIN through VGAM3 HOST TARGET PROTEIN, all of FIG. 1.

It is appreciated that a function of VGR367 gene, herein designated VGR GENE, is inhibition of expression of host target genes, as part of a novel viral mechanism of attacking a host. Accordingly, utilities of VGR367 gene include diagnosis, prevention and treatment of viral infection by Vaccinia Virus. Specific functions, and accordingly utilities, of VGR367 gene correlate with, and may be deduced from, the identity of the host target genes, which are inhibited by VGAM RNAs comprised in the ‘operon-like’ cluster of VGR367 gene: VGAM35 host target protein, VGAM36 host target protein, VGAM37 host target protein, VGAM38 host target protein, VGAM39 host target protein, VGAM40 host target protein, VGAM41 host target protein and VGAM42 host target protein, herein schematically represented by VGAM1 HOST TARGET PROTEIN through VGAM3 HOST TARGET PROTEIN. The function of these host target genes is elaborated hereinabove with reference to VGAM35, VGAM36, VGAM37, VGAM38, VGAM39, VGAM40, VGAM41 and VGAM42. FIG. 9 further provides a conceptual description of novel bioinformatically detected regulatory viral gene, referred to here as Viral Genomic Record 368(VGR368) viral gene, which encodes an ‘operon-like’ cluster of novel viral micro RNA-like genes, each of which in turn modulates expression of at least one host target gene, the function and utility of which at least one host target gene is known in the art.

VGR368 gene, herein designated VGR GENE, is a novel bioinformatically detected regulatory, non protein coding, RNA viral gene. The method by which VGR368 gene was detected is described hereinabove with reference to FIGS. 1-9.

VGR368 gene encodes VGR368 precursor RNA, herein designated VGR PRECURSOR RNA, an RNA molecule, typically several hundred nucleotides long.

VGR368 precursor RNA folds spatially, forming VGR368 folded precursor RNA, herein designated VGR FOLDED PRECURSOR RNA. It is appreciated that VGR368 folded precursor RNA comprises a plurality of what is known in the art as ‘hairpin’ structures. These ‘hairpin’ structures are due to the fact that the nucleotide sequence of VGR368 precursor RNA comprises a plurality of segments, the first half of each such segment having a nucleotide sequence which is at least a partial inversed-reversed sequence of the second half thereof, as is well known in the art.

VGR368 folded precursor RNA is naturally processed by cellular enzymatic activity into at least 8 separate VGAM precursor RNAs, VGAM43 precursor RNA, VGAM44 precursor RNA, VGAM45 precursor RNA, VGAM46 precursor RNA, VGAM47 precursor RNA, VGAM48 precursor RNA, VGAM49 precursor RNA and VGAM50 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM43 RNA, VGAM44 RNA, VGAM45 RNA, VGAM46 RNA, VGAM47 RNA, VGAM48 RNA, VGAM49 RNA and VGAM50 RNA, herein schematically represented by VGAM1 RNA through VGAM3 RNA, each of which VGAM RNAs corresponding to VGAM RNA of FIG. 1.

VGAM43 RNA, herein schematically represented by VGAM1 RNA through VGAM3 RNA, binds complementarily to a host target binding site located in an untranslated region of VGAM43 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM43 host target RNA into VGAM43 host target protein, herein schematically represented by VGAM1 HOST TARGET PROTEIN through VGAM3 HOST TARGET PROTEIN, all of FIG. 1.

VGAM44 RNA, herein schematically represented by VGAM1 RNA through VGAM3 RNA, binds complementarily to a host target binding site located in an untranslated region of VGAM44 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM44 host target RNA into VGAM44 host target protein, herein schematically represented by VGAM1 HOST TARGET PROTEIN through VGAM3 HOST TARGET PROTEIN, all of FIG. 1.

VGAM45 RNA, herein schematically represented by VGAM1 RNA through VGAM3 RNA, binds complementarily to a host target binding site located in an untranslated region of VGAM45 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM45 host target RNA into VGAM45 host target protein, herein schematically represented by VGAM1 HOST TARGET PROTEIN through VGAM3 HOST TARGET PROTEIN, all of FIG. 1.

VGAM46 RNA, herein schematically represented by VGAM1 RNA through VGAM3 RNA, binds complementarily to a host target binding site located in an untranslated region of VGAM46 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM46 host target RNA into VGAM46 host target protein, herein schematically represented by VGAM1 HOST TARGET PROTEIN through VGAM3 HOST TARGET PROTEIN, all of FIG. 1.

VGAM47 RNA, herein schematically represented by VGAM1 RNA through VGAM3 RNA, binds complementarily to a host target binding site located in an untranslated region of VGAM47 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM47 host target RNA into VGAM47 host target protein, herein schematically represented by VGAM1 HOST TARGET PROTEIN through VGAM3 HOST TARGET PROTEIN, all of FIG. 1.

VGAM48 RNA, herein schematically represented by VGAM1 RNA through VGAM3 RNA, binds complementarily to a host target binding site located in an untranslated region of VGAM48 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM48 host target RNA into VGAM48 host target protein, herein schematically represented by VGAM1 HOST TARGET PROTEIN through VGAM3 HOST TARGET PROTEIN, all of FIG. 1.

VGAM49 RNA, herein schematically represented by VGAM1 RNA through VGAM3 RNA, binds complementarily to a host target binding site located in an untranslated region of VGAM49 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM49 host target RNA into VGAM49 host target protein, herein schematically represented by VGAM1 HOST TARGET PROTEIN through VGAM3 HOST TARGET PROTEIN, all of FIG. 1.

VGAM50 RNA, herein schematically represented by VGAM1 RNA through VGAM3 RNA, binds complementarily to a host target binding site located in an untranslated region of VGAM50 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM50 host target RNA into VGAM50 host target protein, herein schematically represented by VGAM1 HOST TARGET PROTEIN through VGAM3 HOST TARGET PROTEIN, all of FIG. 1.

It is appreciated that a function of VGR368 gene, herein designated VGR GENE, is inhibition of expression of host target genes, as part of a novel viral mechanism of attacking a host. Accordingly, utilities of VGR368 gene include diagnosis, prevention and treatment of viral infection by Vaccinia Virus. Specific functions, and accordingly utilities, of VGR368 gene correlate with, and may be deduced from, the identity of the host target genes, which are inhibited by VGAM RNAs comprised in the ‘operon-like’ cluster of VGR368 gene: VGAM43 host target protein, VGAM44 host target protein, VGAM45 host target protein, VGAM46 host target protein, VGAM47 host target protein, VGAM48 host target protein, VGAM49 host target protein and VGAM50 host target protein, herein schematically represented by VGAM1 HOST TARGET PROTEIN through VGAM3 HOST TARGET PROTEIN. The function of these host target genes is elaborated hereinabove with reference to VGAM43, VGAM44, VGAM45, VGAM46, VGAM47, VGAM48, VGAM49 and VGAM50. FIG. 9 further provides a conceptual description of novel bioinformatically detected regulatory viral gene, referred to here as Viral Genomic Record 369(VGR369) viral gene, which encodes an ‘operon-like’ cluster of novel viral micro RNA-like genes, each of which in turn modulates expression of at least one host target gene, the function and utility of which at least one host target gene is known in the art.

VGR369 gene, herein designated VGR GENE, is a novel bioinformatically detected regulatory, non protein coding, RNA viral gene. The method by which VGR369 gene was detected is described hereinabove with reference to FIGS. 1-9.

VGR369 gene encodes VGR369 precursor RNA, herein designated VGR PRECURSOR RNA, an RNA molecule, typically several hundred nucleotides long.

VGR369 precursor RNA folds spatially, forming VGR369 folded precursor RNA, herein designated VGR FOLDED PRECURSOR RNA. It is appreciated that VGR369 folded precursor RNA comprises a plurality of what is known in the art as ‘hairpin’ structures. These ‘hairpin’ structures are due to the fact that the nucleotide sequence of VGR369 precursor RNA comprises a plurality of segments, the first half of each such segment having a nucleotide sequence which is at least a partial inversed-reversed sequence of the second half thereof, as is well known in the art.

VGR369 folded precursor RNA is naturally processed by cellular enzymatic activity into at least 4 separate VGAM precursor RNAs, VGAM51 precursor RNA, VGAM52 precursor RNA, VGAM53 precursor RNA and VGAM54 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM51 RNA, VGAM52 RNA, VGAM53 RNA and VGAM54 RNA, herein schematically represented by VGAM1 RNA through VGAM3 RNA, each of which VGAM RNAs corresponding to VGAM RNA of FIG. 1.

VGAM51 RNA, herein schematically represented by VGAM1 RNA through VGAM3 RNA, binds complementarily to a host target binding site located in an untranslated region of VGAM51 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM51 host target RNA into VGAM51 host target protein, herein schematically represented by VGAM1 HOST TARGET PROTEIN through VGAM3 HOST TARGET PROTEIN, all of FIG. 1.

VGAM52 RNA, herein schematically represented by VGAM1 RNA through VGAM3 RNA, binds complementarily to a host target binding site located in an untranslated region of VGAM52 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM52 host target RNA into VGAM52 host target protein, herein schematically represented by VGAM1 HOST TARGET PROTEIN through VGAM3 HOST TARGET PROTEIN, all of FIG. 1.

VGAM53 RNA, herein schematically represented by VGAM1 RNA through VGAM3 RNA, binds complementarily to a host target binding site located in an untranslated region of VGAM53 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM53 host target RNA into VGAM53 host target protein, herein schematically represented by VGAM1 HOST TARGET PROTEIN through VGAM3 HOST TARGET PROTEIN, all of FIG. 1.

VGAM54 RNA, herein schematically represented by VGAM1 RNA through VGAM3 RNA, binds complementarily to a host target binding site located in an untranslated region of VGAM54 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM54 host target RNA into VGAM54 host target protein, herein schematically represented by VGAM1 HOST TARGET PROTEIN through VGAM3 HOST TARGET PROTEIN, all of FIG. 1.

It is appreciated that a function of VGR369 gene, herein designated VGR GENE, is inhibition of expression of host target genes, as part of a novel viral mechanism of attacking a host. Accordingly, utilities of VGR369 gene include diagnosis, prevention and treatment of viral infection by Vaccinia Virus. Specific functions, and accordingly utilities, of VGR369 gene correlate with, and may be deduced from, the identity of the host target genes, which are inhibited by VGAM RNAs comprised in the ‘operon-like’ cluster of VGR369 gene: VGAM51 host target protein, VGAM52 host target protein, VGAM53 host target protein and VGAM54 host target protein, herein schematically represented by VGAM1 HOST TARGET PROTEIN through VGAM3 HOST TARGET PROTEIN. The function of these host target genes is elaborated hereinabove with reference to VGAM51, VGAM52, VGAM53 and VGAM54. FIG. 9 further provides a conceptual description of novel bioinformatically detected regulatory viral gene, referred to here as Viral Genomic Record 370(VGR370) viral gene, which encodes an ‘operon-like’ cluster of novel viral micro RNA-like genes, each of which in turn modulates expression of at least one host target gene, the function and utility of which at least one host target gene is known in the art.

VGR370 gene, herein designated VGR GENE, is a novel bioinformatically detected regulatory, non protein coding, RNA viral gene. The method by which VGR370 gene was detected is described hereinabove with reference to FIGS. 1-9.

VGR370 gene encodes VGR370 precursor RNA, herein designated VGR PRECURSOR RNA, an RNA molecule, typically several hundred nucleotides long.

VGR370 precursor RNA folds spatially, forming VGR370 folded precursor RNA, herein designated VGR FOLDED PRECURSOR RNA. It is appreciated that VGR370 folded precursor RNA comprises a plurality of what is known in the art as ‘hairpin’ structures. These ‘hairpin’ structures are due to the fact that the nucleotide sequence of VGR370 precursor RNA comprises a plurality of segments, the first half of each such segment having a nucleotide sequence which is at least a partial inversed-reversed sequence of the second half thereof, as is well known in the art.

VGR370 folded precursor RNA is naturally processed by cellular enzymatic activity into at least 2 separate VGAM precursor RNAs, VGAM56 precursor RNA and VGAM57 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM56 RNA and VGAM57 RNA, herein schematically represented by VGAM1 RNA through VGAM3 RNA, each of which VGAM RNAs corresponding to VGAM RNA of FIG. 1.

VGAM56 RNA, herein schematically represented by VGAM1 RNA through VGAM3 RNA, binds complementarily to a host target binding site located in an untranslated region of VGAM56 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM56 host target RNA into VGAM56 host target protein, herein schematically represented by VGAM1 HOST TARGET PROTEIN through VGAM3 HOST TARGET PROTEIN, all of FIG. 1.

VGAM57 RNA, herein schematically represented by VGAM1 RNA through VGAM3 RNA, binds complementarily to a host target binding site located in an untranslated region of VGAM57 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM57 host target RNA into VGAM57 host target protein, herein schematically represented by VGAM1 HOST TARGET PROTEIN through VGAM3 HOST TARGET PROTEIN, all of FIG. 1.

It is appreciated that a function of VGR370 gene, herein designated VGR GENE, is inhibition of expression of host target genes, as part of a novel viral mechanism of attacking a host. Accordingly, utilities of VGR370 gene include diagnosis, prevention and treatment of viral infection by Vaccinia Virus. Specific functions, and accordingly utilities, of VGR370 gene correlate with, and may be deduced from, the identity of the host target genes, which are inhibited by VGAM RNAs comprised in the ‘operon-like’ cluster of VGR370 gene: VGAM56 host target protein and VGAM57 host target protein, herein schematically represented by VGAM1 HOST TARGET PROTEIN through VGAM3 HOST TARGET PROTEIN. The function of these host target genes is elaborated hereinabove with reference to VGAM56 and VGAM57. FIG. 9 further provides a conceptual description of novel bioinformatically detected regulatory viral gene, referred to here as Viral Genomic Record 371(VGR371) viral gene, which encodes an ‘operon-like’ cluster of novel viral micro RNA-like genes, each of which in turn modulates expression of at least one host target gene, the function and utility of which at least one host target gene is known in the art.

VGR371 gene, herein designated VGR GENE, is a novel bioinformatically detected regulatory, non protein coding, RNA viral gene. The method by which VGR371 gene was detected is described hereinabove with reference to FIGS. 1-9.

VGR371 gene encodes VGR371 precursor RNA, herein designated VGR PRECURSOR RNA, an RNA molecule, typically several hundred nucleotides long.

VGR371 precursor RNA folds spatially, forming VGR371 folded precursor RNA, herein designated VGR FOLDED PRECURSOR RNA. It is appreciated that VGR371 folded precursor RNA comprises a plurality of what is known in the art as ‘hairpin’ structures. These ‘hairpin’ structures are due to the fact that the nucleotide sequence of VGR371 precursor RNA comprises a plurality of segments, the first half of each such segment having a nucleotide sequence which is at least a partial inversed-reversed sequence of the second half thereof, as is well known in the art.

VGR371 folded precursor RNA is naturally processed by cellular enzymatic activity into at least 8 separate VGAM precursor RNAs, VGAM58 precursor RNA, VGAM59 precursor RNA, VGAM60 precursor RNA, VGAM61 precursor RNA, VGAM62 precursor RNA, VGAM63 precursor RNA, VGAM64 precursor RNA and VGAM65 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM58 RNA, VGAM59 RNA, VGAM60 RNA, VGAM61 RNA, VGAM62 RNA, VGAM63 RNA, VGAM64 RNA and VGAM65 RNA, herein schematically represented by VGAM1 RNA through VGAM3 RNA, each of which VGAM RNAs corresponding to VGAM RNA of FIG. 1.

VGAM58 RNA, herein schematically represented by VGAM1 RNA through VGAM3 RNA, binds complementarily to a host target binding site located in an untranslated region of VGAM58 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM58 host target RNA into VGAM58 host target protein, herein schematically represented by VGAM1 HOST TARGET PROTEIN through VGAM3 HOST TARGET PROTEIN, all of FIG. 1.

VGAM59 RNA, herein schematically represented by VGAM1 RNA through VGAM3 RNA, binds complementarily to a host target binding site located in an untranslated region of VGAM59 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM59 host target RNA into VGAM59 host target protein, herein schematically represented by VGAM1 HOST TARGET PROTEIN through VGAM3 HOST TARGET PROTEIN, all of FIG. 1.

VGAM60 RNA, herein schematically represented by VGAM1 RNA through VGAM3 RNA, binds complementarily to a host target binding site located in an untranslated region of VGAM60 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM60 host target RNA into VGAM60 host target protein, herein schematically represented by VGAM1 HOST TARGET PROTEIN through VGAM3 HOST TARGET PROTEIN, all of FIG. 1.

VGAM61 RNA, herein schematically represented by VGAM1 RNA through VGAM3 RNA, binds complementarily to a host target binding site located in an untranslated region of VGAM61 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM61 host target RNA into VGAM61 host target protein, herein schematically represented by VGAM1 HOST TARGET PROTEIN through VGAM3 HOST TARGET PROTEIN, all of FIG. 1.

VGAM62 RNA, herein schematically represented by VGAM1 RNA through VGAM3 RNA, binds complementarily to a host target binding site located in an untranslated region of VGAM62 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM62 host target RNA into VGAM62 host target protein, herein schematically represented by VGAM1 HOST TARGET PROTEIN through VGAM3 HOST TARGET PROTEIN, all of FIG. 1.

VGAM63 RNA, herein schematically represented by VGAM1 RNA through VGAM3 RNA, binds complementarily to a host target binding site located in an untranslated region of VGAM63 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM63 host target RNA into VGAM63 host target protein, herein schematically represented by VGAM1 HOST TARGET PROTEIN through VGAM3 HOST TARGET PROTEIN, all of FIG. 1.

VGAM64 RNA, herein schematically represented by VGAM1 RNA through VGAM3 RNA, binds complementarily to a host target binding site located in an untranslated region of VGAM64 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM64 host target RNA into VGAM64 host target protein, herein schematically represented by VGAM1 HOST TARGET PROTEIN through VGAM3 HOST TARGET PROTEIN, all of FIG. 1.

VGAM65 RNA, herein schematically represented by VGAM1 RNA through VGAM3 RNA, binds complementarily to a host target binding site located in an untranslated region of VGAM65 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM65 host target RNA into VGAM65 host target protein, herein schematically represented by VGAM1 HOST TARGET PROTEIN through VGAM3 HOST TARGET PROTEIN, all of FIG. 1.

It is appreciated that a function of VGR371 gene, herein designated VGR GENE, is inhibition of expression of host target genes, as part of a novel viral mechanism of attacking a host. Accordingly, utilities of VGR371 gene include diagnosis, prevention and treatment of viral infection by Vaccinia Virus. Specific functions, and accordingly utilities, of VGR371 gene correlate with, and may be deduced from, the identity of the host target genes, which are inhibited by VGAM RNAs comprised in the ‘operon-like’ cluster of VGR371 gene: VGAM58 host target protein, VGAM59 host target protein, VGAM60 host target protein, VGAM61 host target protein, VGAM62 host target protein, VGAM63 host target protein, VGAM64 host target protein and VGAM65 host target protein, herein schematically represented by VGAM1 HOST TARGET PROTEIN through VGAM3 HOST TARGET PROTEIN. The function of these host target genes is elaborated hereinabove with reference to VGAM58, VGAM59, VGAM60, VGAM61, VGAM62, VGAM63, VGAM64 and VGAM65. FIG. 9 further provides a conceptual description of novel bioinformatically detected regulatory viral gene, referred to here as Viral Genomic Record 372(VGR372) viral gene, which encodes an ‘operon-like’ cluster of novel viral micro RNA-like genes, each of which in turn modulates expression of at least one host target gene, the function and utility of which at least one host target gene is known in the art.

VGR372 gene, herein designated VGR GENE, is a novel bioinformatically detected regulatory, non protein coding, RNA viral gene. The method by which VGR372 gene was detected is described hereinabove with reference to FIGS. 1-9.

VGR372 gene encodes VGR372 precursor RNA, herein designated VGR PRECURSOR RNA, an RNA molecule, typically several hundred nucleotides long.

VGR372 precursor RNA folds spatially, forming VGR372 folded precursor RNA, herein designated VGR FOLDED PRECURSOR RNA. It is appreciated that VGR372 folded precursor RNA comprises a plurality of what is known in the art as ‘hairpin’ structures. These ‘hairpin’ structures are due to the fact that the nucleotide sequence of VGR372 precursor RNA comprises a plurality of segments, the first half of each such segment having a nucleotide sequence which is at least a partial inversed-reversed sequence of the second half thereof, as is well known in the art.

VGR372 folded precursor RNA is naturally processed by cellular enzymatic activity into at least 3 separate VGAM precursor RNAs, VGAM66 precursor RNA, VGAM67 precursor RNA and VGAM68 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM66 RNA, VGAM67 RNA and VGAM68 RNA, herein schematically represented by VGAM1 RNA through VGAM3 RNA, each of which VGAM RNAs corresponding to VGAM RNA of FIG. 1.

VGAM66 RNA, herein schematically represented by VGAM1 RNA through VGAM3 RNA, binds complementarily to a host target binding site located in an untranslated region of VGAM66 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM66 host target RNA into VGAM66 host target protein, herein schematically represented by VGAM1 HOST TARGET PROTEIN through VGAM3 HOST TARGET PROTEIN, all of FIG. 1.

VGAM67 RNA, herein schematically represented by VGAM1 RNA through VGAM3 RNA, binds complementarily to a host target binding site located in an untranslated region of VGAM67 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM67 host target RNA into VGAM67 host target protein, herein schematically represented by VGAM1 HOST TARGET PROTEIN through VGAM3 HOST TARGET PROTEIN, all of FIG. 1.

VGAM68 RNA, herein schematically represented by VGAM1 RNA through VGAM3 RNA, binds complementarily to a host target binding site located in an untranslated region of VGAM68 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM68 host target RNA into VGAM68 host target protein, herein schematically represented by VGAM1 HOST TARGET PROTEIN through VGAM3 HOST TARGET PROTEIN, all of FIG. 1.

It is appreciated that a function of VGR372 gene, herein designated VGR GENE, is inhibition of expression of host target genes, as part of a novel viral mechanism of attacking a host. Accordingly, utilities of VGR372 gene include diagnosis, prevention and treatment of viral infection by Vaccinia Virus. Specific functions, and accordingly utilities, of VGR372 gene correlate with, and may be deduced from, the identity of the host target genes, which are inhibited by VGAM RNAs comprised in the ‘operon-like’ cluster of VGR372 gene: VGAM66 host target protein, VGAM67 host target protein and VGAM68 host target protein, herein schematically represented by VGAM1 HOST TARGET PROTEIN through VGAM3 HOST TARGET PROTEIN. The function of these host target genes is elaborated hereinabove with reference to VGAM66, VGAM67 and VGAM68. FIG. 9 further provides a conceptual description of novel bioinformatically detected regulatory viral gene, referred to here as Viral Genomic Record 373(VGR373) viral gene, which encodes an ‘operon-like’ cluster of novel viral micro RNA-like genes, each of which in turn modulates expression of at least one host target gene, the function and utility of which at least one host target gene is known in the art.

VGR373 gene, herein designated VGR GENE, is a novel bioinformatically detected regulatory, non protein coding, RNA viral gene. The method by which VGR373 gene was detected is described hereinabove with reference to FIGS. 1-9.

VGR373 gene encodes VGR373 precursor RNA, herein designated VGR PRECURSOR RNA, an RNA molecule, typically several hundred nucleotides long.

VGR373 precursor RNA folds spatially, forming VGR373 folded precursor RNA, herein designated VGR FOLDED PRECURSOR RNA. It is appreciated that VGR373 folded precursor RNA comprises a plurality of what is known in the art as ‘hairpin’ structures. These ‘hairpin’ structures are due to the fact that the nucleotide sequence of VGR373 precursor RNA comprises a plurality of segments, the first half of each such segment having a nucleotide sequence which is at least a partial inversed-reversed sequence of the second half thereof, as is well known in the art.

VGR373 folded precursor RNA is naturally processed by cellular enzymatic activity into at least 3 separate VGAM precursor RNAs, VGAM71 precursor RNA, VGAM72 precursor RNA and VGAM73 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM71 RNA, VGAM72 RNA and VGAM73 RNA, herein schematically represented by VGAM1 RNA through VGAM3 RNA, each of which VGAM RNAs corresponding to VGAM RNA of FIG. 1.

VGAM71 RNA, herein schematically represented by VGAM1 RNA through VGAM3 RNA, binds complementarily to a host target binding site located in an untranslated region of VGAM71 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM71 host target RNA into VGAM71 host target protein, herein schematically represented by VGAM1 HOST TARGET PROTEIN through VGAM3 HOST TARGET PROTEIN, all of FIG. 1.

VGAM72 RNA, herein schematically represented by VGAM1 RNA through VGAM3 RNA, binds complementarily to a host target binding site located in an untranslated region of VGAM72 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM72 host target RNA into VGAM72 host target protein, herein schematically represented by VGAM1 HOST TARGET PROTEIN through VGAM3 HOST TARGET PROTEIN, all of FIG. 1.

VGAM73 RNA, herein schematically represented by VGAM1 RNA through VGAM3 RNA, binds complementarily to a host target binding site located in an untranslated region of VGAM73 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM73 host target RNA into VGAM73 host target protein, herein schematically represented by VGAM1 HOST TARGET PROTEIN through VGAM3 HOST TARGET PROTEIN, all of FIG. 1.

It is appreciated that a function of VGR373 gene, herein designated VGR GENE, is inhibition of expression of host target genes, as part of a novel viral mechanism of attacking a host. Accordingly, utilities of VGR373 gene include diagnosis, prevention and treatment of viral infection by Vaccinia Virus. Specific functions, and accordingly utilities, of VGR373 gene correlate with, and may be deduced from, the identity of the host target genes, which are inhibited by VGAM RNAs comprised in the ‘operon-like’ cluster of VGR373 gene: VGAM71 host target protein, VGAM72 host target protein and VGAM73 host target protein, herein schematically represented by VGAM1 HOST TARGET PROTEIN through VGAM3 HOST TARGET PROTEIN. The function of these host target genes is elaborated hereinabove with reference to VGAM71, VGAM72 and VGAM73. FIG. 9 further provides a conceptual description of novel bioinformatically detected regulatory viral gene, referred to here as Viral Genomic Record 374(VGR374) viral gene, which encodes an ‘operon-like’ cluster of novel viral micro RNA-like genes, each of which in turn modulates expression of at least one host target gene, the function and utility of which at least one host target gene is known in the art.

VGR374 gene, herein designated VGR GENE, is a novel bioinformatically detected regulatory, non protein coding, RNA viral gene. The method by which VGR374 gene was detected is described hereinabove with reference to FIGS. 1-9.

VGR374 gene encodes VGR374 precursor RNA, herein designated VGR PRECURSOR RNA, an RNA molecule, typically several hundred nucleotides long.

VGR374 precursor RNA folds spatially, forming VGR374 folded precursor RNA, herein designated VGR FOLDED PRECURSOR RNA. It is appreciated that VGR374 folded precursor RNA comprises a plurality of what is known in the art as ‘hairpin’ structures. These ‘hairpin’ structures are due to the fact that the nucleotide sequence of VGR374 precursor RNA comprises a plurality of segments, the first half of each such segment having a nucleotide sequence which is at least a partial inversed-reversed sequence of the second half thereof, as is well known in the art.

VGR374 folded precursor RNA is naturally processed by cellular enzymatic activity into at least 2 separate VGAM precursor RNAs, VGAM74 precursor RNA and VGAM75 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM74 RNA and VGAM75 RNA, herein schematically represented by VGAM1 RNA through VGAM3 RNA, each of which VGAM RNAs corresponding to VGAM RNA of FIG. 1.

VGAM74 RNA, herein schematically represented by VGAM1 RNA through VGAM3 RNA, binds complementarily to a host target binding site located in an untranslated region of VGAM74 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM74 host target RNA into VGAM74 host target protein, herein schematically represented by VGAM1 HOST TARGET PROTEIN through VGAM3 HOST TARGET PROTEIN, all of FIG. 1.

VGAM75 RNA, herein schematically represented by VGAM1 RNA through VGAM3 RNA, binds complementarily to a host target binding site located in an untranslated region of VGAM75 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM75 host target RNA into VGAM75 host target protein, herein schematically represented by VGAM1 HOST TARGET PROTEIN through VGAM3 HOST TARGET PROTEIN, all of FIG. 1.

It is appreciated that a function of VGR374 gene, herein designated VGR GENE, is inhibition of expression of host target genes, as part of a novel viral mechanism of attacking a host. Accordingly, utilities of VGR374 gene include diagnosis, prevention and treatment of viral infection by Vaccinia Virus. Specific functions, and accordingly utilities, of VGR374 gene correlate with, and may be deduced from, the identity of the host target genes, which are inhibited by VGAM RNAs comprised in the ‘operon-like’ cluster of VGR374 gene: VGAM74 host target protein and VGAM75 host target protein, herein schematically represented by VGAM1 HOST TARGET PROTEIN through VGAM3 HOST TARGET PROTEIN. The function of these host target genes is elaborated hereinabove with reference to VGAM74 and VGAM75. FIG. 9 further provides a conceptual description of novel bioinformatically detected regulatory viral gene, referred to here as Viral Genomic Record 375(VGR375) viral gene, which encodes an ‘operon-like’ cluster of novel viral micro RNA-like genes, each of which in turn modulates expression of at least one host target gene, the function and utility of which at least one host target gene is known in the art.

VGR375 gene, herein designated VGR GENE, is a novel bioinformatically detected regulatory, non protein coding, RNA viral gene. The method by which VGR375 gene was detected is described hereinabove with reference to FIGS. 1-9.

VGR375 gene encodes VGR375 precursor RNA, herein designated VGR PRECURSOR RNA, an RNA molecule, typically several hundred nucleotides long.

VGR375 precursor RNA folds spatially, forming VGR375 folded precursor RNA, herein designated VGR FOLDED PRECURSOR RNA. It is appreciated that VGR375 folded precursor RNA comprises a plurality of what is known in the art as ‘hairpin’ structures. These ‘hairpin’ structures are due to the fact that the nucleotide sequence of VGR375 precursor RNA comprises a plurality of segments, the first half of each such segment having a nucleotide sequence which is at least a partial inversed-reversed sequence of the second half thereof, as is well known in the art.

VGR375 folded precursor RNA is naturally processed by cellular enzymatic activity into at least 3 separate VGAM precursor RNAs, VGAM77 precursor RNA, VGAM78 precursor RNA and VGAM79 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM77 RNA, VGAM78 RNA and VGAM79 RNA, herein schematically represented by VGAM1 RNA through VGAM3 RNA, each of which VGAM RNAs corresponding to VGAM RNA of FIG. 1.

VGAM77 RNA, herein schematically represented by VGAM1 RNA through VGAM3 RNA, binds complementarily to a host target binding site located in an untranslated region of VGAM77 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM77 host target RNA into VGAM77 host target protein, herein schematically represented by VGAM1 HOST TARGET PROTEIN through VGAM3 HOST TARGET PROTEIN, all of FIG. 1.

VGAM78 RNA, herein schematically represented by VGAM1 RNA through VGAM3 RNA, binds complementarily to a host target binding site located in an untranslated region of VGAM78 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM78 host target RNA into VGAM78 host target protein, herein schematically represented by VGAM1 HOST TARGET PROTEIN through VGAM3 HOST TARGET PROTEIN, all of FIG. 1.

VGAM79 RNA, herein schematically represented by VGAM1 RNA through VGAM3 RNA, binds complementarily to a host target binding site located in an untranslated region of VGAM79 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM79 host target RNA into VGAM79 host target protein, herein schematically represented by VGAM1 HOST TARGET PROTEIN through VGAM3 HOST TARGET PROTEIN, all of FIG. 1.

It is appreciated that a function of VGR375 gene, herein designated VGR GENE, is inhibition of expression of host target genes, as part of a novel viral mechanism of attacking a host. Accordingly, utilities of VGR375 gene include diagnosis, prevention and treatment of viral infection by Vaccinia Virus. Specific functions, and accordingly utilities, of VGR375 gene correlate with, and may be deduced from, the identity of the host target genes, which are inhibited by VGAM RNAs comprised in the ‘operon-like’ cluster of VGR375 gene: VGAM77 host target protein, VGAM78 host target protein and VGAM79 host target protein, herein schematically represented by VGAM1 HOST TARGET PROTEIN through VGAM3 HOST TARGET PROTEIN. The function of these host target genes is elaborated hereinabove with reference to VGAM77, VGAM78 and VGAM79. FIG. 9 further provides a conceptual description of novel bioinformatically detected regulatory viral gene, referred to here as Viral Genomic Record 376(VGR376) viral gene, which encodes an ‘operon-like’ cluster of novel viral micro RNA-like genes, each of which in turn modulates expression of at least one host target gene, the function and utility of which at least one host target gene is known in the art.

VGR376 gene, herein designated VGR GENE, is a novel bioinformatically detected regulatory, non protein coding, RNA viral gene. The method by which VGR376 gene was detected is described hereinabove with reference to FIGS. 1-9.

VGR376 gene encodes VGR376 precursor RNA, herein designated VGR PRECURSOR RNA, an RNA molecule, typically several hundred nucleotides long.

VGR376 precursor RNA folds spatially, forming VGR376 folded precursor RNA, herein designated VGR FOLDED PRECURSOR RNA. It is appreciated that VGR376 folded precursor RNA comprises a plurality of what is known in the art as ‘hairpin’ structures. These ‘hairpin’ structures are due to the fact that the nucleotide sequence of VGR376 precursor RNA comprises a plurality of segments, the first half of each such segment having a nucleotide sequence which is at least a partial inversed-reversed sequence of the second half thereof, as is well known in the art.

VGR376 folded precursor RNA is naturally processed by cellular enzymatic activity into at least 8 separate VGAM precursor RNAs, VGAM81 precursor RNA, VGAM82 precursor RNA, VGAM83 precursor RNA, VGAM84 precursor RNA, VGAM85 precursor RNA, VGAM86 precursor RNA, VGAM87 precursor RNA and VGAM88 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM81 RNA, VGAM82 RNA, VGAM83 RNA, VGAM84 RNA, VGAM85 RNA, VGAM86 RNA, VGAM87 RNA and VGAM88 RNA, herein schematically represented by VGAM1 RNA through VGAM3 RNA, each of which VGAM RNAs corresponding to VGAM RNA of FIG. 1.

VGAM81 RNA, herein schematically represented by VGAM1 RNA through VGAM3 RNA, binds complementarily to a host target binding site located in an untranslated region of VGAM81 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM81 host target RNA into VGAM81 host target protein, herein schematically represented by VGAM1 HOST TARGET PROTEIN through VGAM3 HOST TARGET PROTEIN, all of FIG. 1.

VGAM82 RNA, herein schematically represented by VGAM1 RNA through VGAM3 RNA, binds complementarily to a host target binding site located in an untranslated region of VGAM82 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM82 host target RNA into VGAM82 host target protein, herein schematically represented by VGAM1 HOST TARGET PROTEIN through VGAM3 HOST TARGET PROTEIN, all of FIG. 1.

VGAM83 RNA, herein schematically represented by VGAM1 RNA through VGAM3 RNA, binds complementarily to a host target binding site located in an untranslated region of VGAM83 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM83 host target RNA into VGAM83 host target protein, herein schematically represented by VGAM1 HOST TARGET PROTEIN through VGAM3 HOST TARGET PROTEIN, all of FIG. 1.

VGAM84 RNA, herein schematically represented by VGAM1 RNA through VGAM3 RNA, binds complementarily to a host target binding site located in an untranslated region of VGAM84 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM84 host target RNA into VGAM84 host target protein, herein schematically represented by VGAM1 HOST TARGET PROTEIN through VGAM3 HOST TARGET PROTEIN, all of FIG. 1.

VGAM85 RNA, herein schematically represented by VGAM1 RNA through VGAM3 RNA, binds complementarily to a host target binding site located in an untranslated region of VGAM85 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM85 host target RNA into VGAM85 host target protein, herein schematically represented by VGAM1 HOST TARGET PROTEIN through VGAM3 HOST TARGET PROTEIN, all of FIG. 1.

VGAM86 RNA, herein schematically represented by VGAM1 RNA through VGAM3 RNA, binds complementarily to a host target binding site located in an untranslated region of VGAM86 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM86 host target RNA into VGAM86 host target protein, herein schematically represented by VGAM1 HOST TARGET PROTEIN through VGAM3 HOST TARGET PROTEIN, all of FIG. 1.

VGAM87 RNA, herein schematically represented by VGAM1 RNA through VGAM3 RNA, binds complementarily to a host target binding site located in an untranslated region of VGAM87 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM87 host target RNA into VGAM87 host target protein, herein schematically represented by VGAM1 HOST TARGET PROTEIN through VGAM3 HOST TARGET PROTEIN, all of FIG. 1.

VGAM88 RNA, herein schematically represented by VGAM1 RNA through VGAM3 RNA, binds complementarily to a host target binding site located in an untranslated region of VGAM88 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM88 host target RNA into VGAM88 host target protein, herein schematically represented by VGAM1 HOST TARGET PROTEIN through VGAM3 HOST TARGET PROTEIN, all of FIG. 1.

It is appreciated that a function of VGR376 gene, herein designated VGR GENE, is inhibition of expression of host target genes, as part of a novel viral mechanism of attacking a host. Accordingly, utilities of VGR376 gene include diagnosis, prevention and treatment of viral infection by Vaccinia Virus. Specific functions, and accordingly utilities, of VGR376 gene correlate with, and may be deduced from, the identity of the host target genes, which are inhibited by VGAM RNAs comprised in the ‘operon-like’ cluster of VGR376 gene: VGAM81 host target protein, VGAM82 host target protein, VGAM83 host target protein, VGAM84 host target protein, VGAM85 host target protein, VGAM86 host target protein, VGAM87 host target protein and VGAM88 host target protein, herein schematically represented by VGAM1 HOST TARGET PROTEIN through VGAM3 HOST TARGET PROTEIN. The function of these host target genes is elaborated hereinabove with reference to VGAM81, VGAM82, VGAM83, VGAM84, VGAM85, VGAM86, VGAM87 and VGAM88. FIG. 9 further provides a conceptual description of novel bioinformatically detected regulatory viral gene, referred to here as Viral Genomic Record 377(VGR377) viral gene, which encodes an ‘operon-like’ cluster of novel viral micro RNA-like genes, each of which in turn modulates expression of at least one host target gene, the function and utility of which at least one host target gene is known in the art.

VGR377 gene, herein designated VGR GENE, is a novel bioinformatically detected regulatory, non protein coding, RNA viral gene. The method by which VGR377 gene was detected is described hereinabove with reference to FIGS. 1-9.

VGR377 gene encodes VGR377 precursor RNA, herein designated VGR PRECURSOR RNA, an RNA molecule, typically several hundred nucleotides long.

VGR377 precursor RNA folds spatially, forming VGR377 folded precursor RNA, herein designated VGR FOLDED PRECURSOR RNA. It is appreciated that VGR377 folded precursor RNA comprises a plurality of what is known in the art as ‘hairpin’ structures. These ‘hairpin’ structures are due to the fact that the nucleotide sequence of VGR377 precursor RNA comprises a plurality of segments, the first half of each such segment having a nucleotide sequence which is at least a partial inversed-reversed sequence of the second half thereof, as is well known in the art.

VGR377 folded precursor RNA is naturally processed by cellular enzymatic activity into at least 7 separate VGAM precursor RNAs, VGAM91 precursor RNA, VGAM92 precursor RNA, VGAM93 precursor RNA, VGAM94 precursor RNA, VGAM95 precursor RNA, VGAM96 precursor RNA and VGAM97 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM91 RNA, VGAM92 RNA, VGAM93 RNA, VGAM94 RNA, VGAM95 RNA, VGAM96 RNA and VGAM97 RNA, herein schematically represented by VGAM1 RNA through VGAM3 RNA, each of which VGAM RNAs corresponding to VGAM RNA of FIG. 1.

VGAM91 RNA, herein schematically represented by VGAM1 RNA through VGAM3 RNA, binds complementarily to a host target binding site located in an untranslated region of VGAM91 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM91 host target RNA into VGAM91 host target protein, herein schematically represented by VGAM1 HOST TARGET PROTEIN through VGAM3 HOST TARGET PROTEIN, all of FIG. 1.

VGAM92 RNA, herein schematically represented by VGAM1 RNA through VGAM3 RNA, binds complementarily to a host target binding site located in an untranslated region of VGAM92 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM92 host target RNA into VGAM92 host target protein, herein schematically represented by VGAM1 HOST TARGET PROTEIN through VGAM3 HOST TARGET PROTEIN, all of FIG. 1.

VGAM93 RNA, herein schematically represented by VGAM1 RNA through VGAM3 RNA, binds complementarily to a host target binding site located in an untranslated region of VGAM93 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM93 host target RNA into VGAM93 host target protein, herein schematically represented by VGAM1 HOST TARGET PROTEIN through VGAM3 HOST TARGET PROTEIN, all of FIG. 1.

VGAM94 RNA, herein schematically represented by VGAM1 RNA through VGAM3 RNA, binds complementarily to a host target binding site located in an untranslated region of VGAM94 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM94 host target RNA into VGAM94 host target protein, herein schematically represented by VGAM1 HOST TARGET PROTEIN through VGAM3 HOST TARGET PROTEIN, all of FIG. 1.

VGAM95 RNA, herein schematically represented by VGAM1 RNA through VGAM3 RNA, binds complementarily to a host target binding site located in an untranslated region of VGAM95 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM95 host target RNA into VGAM95 host target protein, herein schematically represented by VGAM1 HOST TARGET PROTEIN through VGAM3 HOST TARGET PROTEIN, all of FIG. 1.

VGAM96 RNA, herein schematically represented by VGAM1 RNA through VGAM3 RNA, binds complementarily to a host target binding site located in an untranslated region of VGAM96 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM96 host target RNA into VGAM96 host target protein, herein schematically represented by VGAM1 HOST TARGET PROTEIN through VGAM3 HOST TARGET PROTEIN, all of FIG. 1.

VGAM97 RNA, herein schematically represented by VGAM1 RNA through VGAM3 RNA, binds complementarily to a host target binding site located in an untranslated region of VGAM97 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM97 host target RNA into VGAM97 host target protein, herein schematically represented by VGAM1 HOST TARGET PROTEIN through VGAM3 HOST TARGET PROTEIN, all of FIG. 1.

It is appreciated that a function of VGR377 gene, herein designated VGR GENE, is inhibition of expression of host target genes, as part of a novel viral mechanism of attacking a host. Accordingly, utilities of VGR377 gene include diagnosis, prevention and treatment of viral infection by Vaccinia Virus. Specific functions, and accordingly utilities, of VGR377 gene correlate with, and may be deduced from, the identity of the host target genes, which are inhibited by VGAM RNAs comprised in the ‘operon-like’ cluster of VGR377 gene: VGAM91 host target protein, VGAM92 host target protein, VGAM93 host target protein, VGAM94 host target protein, VGAM95 host target protein, VGAM96 host target protein and VGAM97 host target protein, herein schematically represented by VGAM1 HOST TARGET PROTEIN through VGAM3 HOST TARGET PROTEIN. The function of these host target genes is elaborated hereinabove with reference to VGAM91, VGAM92, VGAM93, VGAM94, VGAM95, VGAM96 and VGAM97. FIG. 9 further provides a conceptual description of novel bioinformatically detected regulatory viral gene, referred to here as Viral Genomic Record 378(VGR378) viral gene, which encodes an ‘operon-like’ cluster of novel viral micro RNA-like genes, each of which in turn modulates expression of at least one host target gene, the function and utility of which at least one host target gene is known in the art.

VGR378 gene, herein designated VGR GENE, is a novel bioinformatically detected regulatory, non protein coding, RNA viral gene. The method by which VGR378 gene was detected is described hereinabove with reference to FIGS. 1-9.

VGR378 gene encodes VGR378 precursor RNA, herein designated VGR PRECURSOR RNA, an RNA molecule, typically several hundred nucleotides long.

VGR378 precursor RNA folds spatially, forming VGR378 folded precursor RNA, herein designated VGR FOLDED PRECURSOR RNA. It is appreciated that VGR378 folded precursor RNA comprises a plurality of what is known in the art as ‘hairpin’ structures. These ‘hairpin’ structures are due to the fact that the nucleotide sequence of VGR378 precursor RNA comprises a plurality of segments, the first half of each such segment having a nucleotide sequence which is at least a partial inversed-reversed sequence of the second half thereof, as is well known in the art.

VGR378 folded precursor RNA is naturally processed by cellular enzymatic activity into at least 2 separate VGAM precursor RNAs, VGAM99 precursor RNA and VGAM100 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM99 RNA and VGAM100 RNA, herein schematically represented by VGAM1 RNA through VGAM3 RNA, each of which VGAM RNAs corresponding to VGAM RNA of FIG. 1.

VGAM99 RNA, herein schematically represented by VGAM1 RNA through VGAM3 RNA, binds complementarily to a host target binding site located in an untranslated region of VGAM99 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM99 host target RNA into VGAM99 host target protein, herein schematically represented by VGAM1 HOST TARGET PROTEIN through VGAM3 HOST TARGET PROTEIN, all of FIG. 1.

VGAM100 RNA, herein schematically represented by VGAM1 RNA through VGAM3 RNA, binds complementarily to a host target binding site located in an untranslated region of VGAM100 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM100 host target RNA into VGAM100 host target protein, herein schematically represented by VGAM1 HOST TARGET PROTEIN through VGAM3 HOST TARGET PROTEIN, all of FIG. 1.

It is appreciated that a function of VGR378 gene, herein designated VGR GENE, is inhibition of expression of host target genes, as part of a novel viral mechanism of attacking a host. Accordingly, utilities of VGR378 gene include diagnosis, prevention and treatment of viral infection by Vaccinia Virus. Specific functions, and accordingly utilities, of VGR378 gene correlate with, and may be deduced from, the identity of the host target genes, which are inhibited by VGAM RNAs comprised in the ‘operon-like’ cluster of VGR378 gene: VGAM99 host target protein and VGAM100 host target protein, herein schematically represented by VGAM1 HOST TARGET PROTEIN through VGAM3 HOST TARGET PROTEIN. The function of these host target genes is elaborated hereinabove with reference to VGAM99 and VGAM100. FIG. 9 further provides a conceptual description of novel bioinformatically detected regulatory viral gene, referred to here as Viral Genomic Record 379(VGR379) viral gene, which encodes an ‘operon-like’ cluster of novel viral micro RNA-like genes, each of which in turn modulates expression of at least one host target gene, the function and utility of which at least one host target gene is known in the art.

VGR379 gene, herein designated VGR GENE, is a novel bioinformatically detected regulatory, non protein coding, RNA viral gene. The method by which VGR379 gene was detected is described hereinabove with reference to FIGS. 1-9.

VGR379 gene encodes VGR379 precursor RNA, herein designated VGR PRECURSOR RNA, an RNA molecule, typically several hundred nucleotides long.

VGR379 precursor RNA folds spatially, forming VGR379 folded precursor RNA, herein designated VGR FOLDED PRECURSOR RNA. It is appreciated that VGR379 folded precursor RNA comprises a plurality of what is known in the art as ‘hairpin’ structures. These ‘hairpin’ structures are due to the fact that the nucleotide sequence of VGR379 precursor RNA comprises a plurality of segments, the first half of each such segment having a nucleotide sequence which is at least a partial inversed-reversed sequence of the second half thereof, as is well known in the art.

VGR379 folded precursor RNA is naturally processed by cellular enzymatic activity into at least 3 separate VGAM precursor RNAs, VGAM101 precursor RNA, VGAM102 precursor RNA and VGAM103 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM101 RNA, VGAM102 RNA and VGAM103 RNA, herein schematically represented by VGAM1 RNA through VGAM3 RNA, each of which VGAM RNAs corresponding to VGAM RNA of FIG. 1.

VGAM101 RNA, herein schematically represented by VGAM1 RNA through VGAM3 RNA, binds complementarily to a host target binding site located in an untranslated region of VGAM101 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM101 host target RNA into VGAM101 host target protein, herein schematically represented by VGAM1 HOST TARGET PROTEIN through VGAM3 HOST TARGET PROTEIN, all of FIG. 1.

VGAM102 RNA, herein schematically represented by VGAM1 RNA through VGAM3 RNA, binds complementarily to a host target binding site located in an untranslated region of VGAM102 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM102 host target RNA into VGAM102 host target protein, herein schematically represented by VGAM1 HOST TARGET PROTEIN through VGAM3 HOST TARGET PROTEIN, all of FIG. 1.

VGAM103 RNA, herein schematically represented by VGAM1 RNA through VGAM3 RNA, binds complementarily to a host target binding site located in an untranslated region of VGAM103 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM103 host target RNA into VGAM103 host target protein, herein schematically represented by VGAM1 HOST TARGET PROTEIN through VGAM3 HOST TARGET PROTEIN, all of FIG. 1.

It is appreciated that a function of VGR379 gene, herein designated VGR GENE, is inhibition of expression of host target genes, as part of a novel viral mechanism of attacking a host. Accordingly, utilities of VGR379 gene include diagnosis, prevention and treatment of viral infection by Vaccinia Virus. Specific functions, and accordingly utilities, of VGR379 gene correlate with, and may be deduced from, the identity of the host target genes, which are inhibited by VGAM RNAs comprised in the ‘operon-like’ cluster of VGR379 gene: VGAM101 host target protein, VGAM102 host target protein and VGAM103 host target protein, herein schematically represented by VGAM1 HOST TARGET PROTEIN through VGAM3 HOST TARGET PROTEIN. The function of these host target genes is elaborated hereinabove with reference to VGAM101, VGAM102 and VGAM103. FIG. 9 further provides a conceptual description of novel bioinformatically detected regulatory viral gene, referred to here as Viral Genomic Record 380(VGR380) viral gene, which encodes an ‘operon-like’ cluster of novel viral micro RNA-like genes, each of which in turn modulates expression of at least one host target gene, the function and utility of which at least one host target gene is known in the art.

VGR380 gene, herein designated VGR GENE, is a novel bioinformatically detected regulatory, non protein coding, RNA viral gene. The method by which VGR380 gene was detected is described hereinabove with reference to FIGS. 1-9.

VGR380 gene encodes VGR380 precursor RNA, herein designated VGR PRECURSOR RNA, an RNA molecule, typically several hundred nucleotides long.

VGR380 precursor RNA folds spatially, forming VGR380 folded precursor RNA, herein designated VGR FOLDED PRECURSOR RNA. It is appreciated that VGR380 folded precursor RNA comprises a plurality of what is known in the art as ‘hairpin’ structures. These ‘hairpin’ structures are due to the fact that the nucleotide sequence of VGR380 precursor RNA comprises a plurality of segments, the first half of each such segment having a nucleotide sequence which is at least a partial inversed-reversed sequence of the second half thereof, as is well known in the art.

VGR380 folded precursor RNA is naturally processed by cellular enzymatic activity into at least 5 separate VGAM precursor RNAs, VGAM104 precursor RNA, VGAM105 precursor RNA, VGAM106 precursor RNA, VGAM107 precursor RNA and VGAM108 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM104 RNA, VGAM105 RNA, VGAM106 RNA, VGAM107 RNA and VGAM108 RNA, herein schematically represented by VGAM1 RNA through VGAM3 RNA, each of which VGAM RNAs corresponding to VGAM RNA of FIG. 1.

VGAM104 RNA, herein schematically represented by VGAM1 RNA through VGAM3 RNA, binds complementarily to a host target binding site located in an untranslated region of VGAM104 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM104 host target RNA into VGAM104 host target protein, herein schematically represented by VGAM1 HOST TARGET PROTEIN through VGAM3 HOST TARGET PROTEIN, all of FIG. 1.

VGAM105 RNA, herein schematically represented by VGAM1 RNA through VGAM3 RNA, binds complementarily to a host target binding site located in an untranslated region of VGAM105 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM105 host target RNA into VGAM105 host target protein, herein schematically represented by VGAM1 HOST TARGET PROTEIN through VGAM3 HOST TARGET PROTEIN, all of FIG. 1.

VGAM106 RNA, herein schematically represented by VGAM1 RNA through VGAM3 RNA, binds complementarily to a host target binding site located in an untranslated region of VGAM106 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM106 host target RNA into VGAM106 host target protein, herein schematically represented by VGAM1 HOST TARGET PROTEIN through VGAM3 HOST TARGET PROTEIN, all of FIG. 1.

VGAM107 RNA, herein schematically represented by VGAM1 RNA through VGAM3 RNA, binds complementarily to a host target binding site located in an untranslated region of VGAM107 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM107 host target RNA into VGAM107 host target protein, herein schematically represented by VGAM1 HOST TARGET PROTEIN through VGAM3 HOST TARGET PROTEIN, all of FIG. 1.

VGAM108 RNA, herein schematically represented by VGAM1 RNA through VGAM3 RNA, binds complementarily to a host target binding site located in an untranslated region of VGAM108 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM108 host target RNA into VGAM108 host target protein, herein schematically represented by VGAM1 HOST TARGET PROTEIN through VGAM3 HOST TARGET PROTEIN, all of FIG. 1.

It is appreciated that a function of VGR380 gene, herein designated VGR GENE, is inhibition of expression of host target genes, as part of a novel viral mechanism of attacking a host. Accordingly, utilities of VGR380 gene include diagnosis, prevention and treatment of viral infection by Vaccinia Virus. Specific functions, and accordingly utilities, of VGR380 gene correlate with, and may be deduced from, the identity of the host target genes, which are inhibited by VGAM RNAs comprised in the ‘operon-like’ cluster of VGR380 gene: VGAM104 host target protein, VGAM105 host target protein, VGAM106 host target protein, VGAM107 host target protein and VGAM108 host target protein, herein schematically represented by VGAM1 HOST TARGET PROTEIN through VGAM3 HOST TARGET PROTEIN. The function of these host target genes is elaborated hereinabove with reference to VGAM104, VGAM105, VGAM106, VGAM107 and VGAM108. FIG. 9 further provides a conceptual description of novel bioinformatically detected regulatory viral gene, referred to here as Viral Genomic Record 381(VGR381) viral gene, which encodes an ‘operon-like’ cluster of novel viral micro RNA-like genes, each of which in turn modulates expression of at least one host target gene, the function and utility of which at least one host target gene is known in the art.

VGR381 gene, herein designated VGR GENE, is a novel bioinformatically detected regulatory, non protein coding, RNA viral gene. The method by which VGR381 gene was detected is described hereinabove with reference to FIGS. 1-9.

VGR381 gene encodes VGR381 precursor RNA, herein designated VGR PRECURSOR RNA, an RNA molecule, typically several hundred nucleotides long.

VGR381 precursor RNA folds spatially, forming VGR381 folded precursor RNA, herein designated VGR FOLDED PRECURSOR RNA. It is appreciated that VGR381 folded precursor RNA comprises a plurality of what is known in the art as ‘hairpin’ structures. These ‘hairpin’ structures are due to the fact that the nucleotide sequence of VGR381 precursor RNA comprises a plurality of segments, the first half of each such segment having a nucleotide sequence which is at least a partial inversed-reversed sequence of the second half thereof, as is well known in the art.

VGR381 folded precursor RNA is naturally processed by cellular enzymatic activity into at least 8 separate VGAM precursor RNAs, VGAM110 precursor RNA, VGAM111 precursor RNA, VGAM112 precursor RNA, VGAM113 precursor RNA, VGAM114 precursor RNA, VGAM115 precursor RNA, VGAM116 precursor RNA and VGAM117 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM110 RNA, VGAM111 RNA, VGAM112 RNA, VGAM113 RNA, VGAM114 RNA, VGAM115 RNA, VGAM116 RNA and VGAM117 RNA, herein schematically represented by VGAM1 RNA through VGAM3 RNA, each of which VGAM RNAs corresponding to VGAM RNA of FIG. 1.

VGAM110 RNA, herein schematically represented by VGAM1 RNA through VGAM3 RNA, binds complementarily to a host target binding site located in an untranslated region of VGAM110 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM110 host target RNA into VGAM110 host target protein, herein schematically represented by VGAM1 HOST TARGET PROTEIN through VGAM3 HOST TARGET PROTEIN, all of FIG. 1.

VGAM111 RNA, herein schematically represented by VGAM1 RNA through VGAM3 RNA, binds complementarily to a host target binding site located in an untranslated region of VGAM111 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM111 host target RNA into VGAM111 host target protein, herein schematically represented by VGAM1 HOST TARGET PROTEIN through VGAM3 HOST TARGET PROTEIN, all of FIG. 1.

VGAM112 RNA, herein schematically represented by VGAM1 RNA through VGAM3 RNA, binds complementarily to a host target binding site located in an untranslated region of VGAM112 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM112 host target RNA into VGAM112 host target protein, herein schematically represented by VGAM1 HOST TARGET PROTEIN through VGAM3 HOST TARGET PROTEIN, all of FIG. 1.

VGAM113 RNA, herein schematically represented by VGAM1 RNA through VGAM3 RNA, binds complementarily to a host target binding site located in an untranslated region of VGAM113 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM113 host target RNA into VGAM113 host target protein, herein schematically represented by VGAM1 HOST TARGET PROTEIN through VGAM3 HOST TARGET PROTEIN, all of FIG. 1.

VGAM114 RNA, herein schematically represented by VGAM1 RNA through VGAM3 RNA, binds complementarily to a host target binding site located in an untranslated region of VGAM114 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM114 host target RNA into VGAM114 host target protein, herein schematically represented by VGAM1 HOST TARGET PROTEIN through VGAM3 HOST TARGET PROTEIN, all of FIG. 1.

VGAM115 RNA, herein schematically represented by VGAM1 RNA through VGAM3 RNA, binds complementarily to a host target binding site located in an untranslated region of VGAM115 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM115 host target RNA into VGAM115 host target protein, herein schematically represented by VGAM1 HOST TARGET PROTEIN through VGAM3 HOST TARGET PROTEIN, all of FIG. 1.

VGAM116 RNA, herein schematically represented by VGAM1 RNA through VGAM3 RNA, binds complementarily to a host target binding site located in an untranslated region of VGAM116 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM116 host target RNA into VGAM116 host target protein, herein schematically represented by VGAM1 HOST TARGET PROTEIN through VGAM3 HOST TARGET PROTEIN, all of FIG. 1.

VGAM117 RNA, herein schematically represented by VGAM1 RNA through VGAM3 RNA, binds complementarily to a host target binding site located in an untranslated region of VGAM117 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM117 host target RNA into VGAM117 host target protein, herein schematically represented by VGAM1 HOST TARGET PROTEIN through VGAM3 HOST TARGET PROTEIN, all of FIG. 1.

It is appreciated that a function of VGR381 gene, herein designated VGR GENE, is inhibition of expression of host target genes, as part of a novel viral mechanism of attacking a host. Accordingly, utilities of VGR381 gene include diagnosis, prevention and treatment of viral infection by Vaccinia Virus. Specific functions, and accordingly utilities, of VGR381 gene correlate with, and may be deduced from, the identity of the host target genes, which are inhibited by VGAM RNAs comprised in the ‘operon-like’ cluster of VGR381 gene: VGAM110 host target protein, VGAM111 host target protein, VGAM112 host target protein, VGAM113 host target protein, VGAM114 host target protein, VGAM115 host target protein, VGAM116 host target protein and VGAM117 host target protein, herein schematically represented by VGAM1 HOST TARGET PROTEIN through VGAM3 HOST TARGET PROTEIN. The function of these host target genes is elaborated hereinabove with reference to VGAM110, VGAM111, VGAM112, VGAM113, VGAM114, VGAM115, VGAM116 and VGAM117. FIG. 9 further provides a conceptual description of novel bioinformatically detected regulatory viral gene, referred to here as Viral Genomic Record 382(VGR382) viral gene, which encodes an ‘operon-like’ cluster of novel viral micro RNA-like genes, each of which in turn modulates expression of at least one host target gene, the function and utility of which at least one host target gene is known in the art.

VGR382 gene, herein designated VGR GENE, is a novel bioinformatically detected regulatory, non protein coding, RNA viral gene. The method by which VGR382 gene was detected is described hereinabove with reference to FIGS. 1-9.

VGR382 gene encodes VGR382 precursor RNA, herein designated VGR PRECURSOR RNA, an RNA molecule, typically several hundred nucleotides long.

VGR382 precursor RNA folds spatially, forming VGR382 folded precursor RNA, herein designated VGR FOLDED PRECURSOR RNA. It is appreciated that VGR382 folded precursor RNA comprises a plurality of what is known in the art as ‘hairpin’ structures. These ‘hairpin’ structures are due to the fact that the nucleotide sequence of VGR382 precursor RNA comprises a plurality of segments, the first half of each such segment having a nucleotide sequence which is at least a partial inversed-reversed sequence of the second half thereof, as is well known in the art.

VGR382 folded precursor RNA is naturally processed by cellular enzymatic activity into at least 4 separate VGAM precursor RNAs, VGAM118 precursor RNA, VGAM119 precursor RNA, VGAM120 precursor RNA and VGAM121 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM118 RNA, VGAM119 RNA, VGAM120 RNA and VGAM121 RNA, herein schematically represented by VGAM1 RNA through VGAM3 RNA, each of which VGAM RNAs corresponding to VGAM RNA of FIG. 1.

VGAM118 RNA, herein schematically represented by VGAM1 RNA through VGAM3 RNA, binds complementarily to a host target binding site located in an untranslated region of VGAM118 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM118 host target RNA into VGAM118 host target protein, herein schematically represented by VGAM1 HOST TARGET PROTEIN through VGAM3 HOST TARGET PROTEIN, all of FIG. 1.

VGAM119 RNA, herein schematically represented by VGAM1 RNA through VGAM3 RNA, binds complementarily to a host target binding site located in an untranslated region of VGAM119 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM119 host target RNA into VGAM119 host target protein, herein schematically represented by VGAM1 HOST TARGET PROTEIN through VGAM3 HOST TARGET PROTEIN, all of FIG. 1.

VGAM120 RNA, herein schematically represented by VGAM1 RNA through VGAM3 RNA, binds complementarily to a host target binding site located in an untranslated region of VGAM120 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM120 host target RNA into VGAM120 host target protein, herein schematically represented by VGAM1 HOST TARGET PROTEIN through VGAM3 HOST TARGET PROTEIN, all of FIG. 1.

VGAM121 RNA, herein schematically represented by VGAM1 RNA through VGAM3 RNA, binds complementarily to a host target binding site located in an untranslated region of VGAM121 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM121 host target RNA into VGAM121 host target protein, herein schematically represented by VGAM1 HOST TARGET PROTEIN through VGAM3 HOST TARGET PROTEIN, all of FIG. 1.

It is appreciated that a function of VGR382 gene, herein designated VGR GENE, is inhibition of expression of host target genes, as part of a novel viral mechanism of attacking a host. Accordingly, utilities of VGR382 gene include diagnosis, prevention and treatment of viral infection by Vaccinia Virus. Specific functions, and accordingly utilities, of VGR382 gene correlate with, and may be deduced from, the identity of the host target genes, which are inhibited by VGAM RNAs comprised in the ‘operon-like’ cluster of VGR382 gene: VGAM118 host target protein, VGAM119 host target protein, VGAM120 host target protein and VGAM121 host target protein, herein schematically represented by VGAM1 HOST TARGET PROTEIN through VGAM3 HOST TARGET PROTEIN. The function of these host target genes is elaborated hereinabove with reference to VGAM118, VGAM119, VGAM120 and VGAM121. FIG. 9 further provides a conceptual description of novel bioinformatically detected regulatory viral gene, referred to here as Viral Genomic Record 383(VGR383) viral gene, which encodes an ‘operon-like’ cluster of novel viral micro RNA-like genes, each of which in turn modulates expression of at least one host target gene, the function and utility of which at least one host target gene is known in the art.

VGR383 gene, herein designated VGR GENE, is a novel bioinformatically detected regulatory, non protein coding, RNA viral gene. The method by which VGR383 gene was detected is described hereinabove with reference to FIGS. 1-9.

VGR383 gene encodes VGR383 precursor RNA, herein designated VGR PRECURSOR RNA, an RNA molecule, typically several hundred nucleotides long.

VGR383 precursor RNA folds spatially, forming VGR383 folded precursor RNA, herein designated VGR FOLDED PRECURSOR RNA. It is appreciated that VGR383 folded precursor RNA comprises a plurality of what is known in the art as ‘hairpin’ structures. These ‘hairpin’ structures are due to the fact that the nucleotide sequence of VGR383 precursor RNA comprises a plurality of segments, the first half of each such segment having a nucleotide sequence which is at least a partial inversed-reversed sequence of the second half thereof, as is well known in the art.

VGR383 folded precursor RNA is naturally processed by cellular enzymatic activity into at least 2 separate VGAM precursor RNAs, VGAM123 precursor RNA and VGAM124 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM123 RNA and VGAM124 RNA, herein schematically represented by VGAM1 RNA through VGAM3 RNA, each of which VGAM RNAs corresponding to VGAM RNA of FIG. 1.

VGAM123 RNA, herein schematically represented by VGAM1 RNA through VGAM3 RNA, binds complementarily to a host target binding site located in an untranslated region of VGAM123 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM123 host target RNA into VGAM123 host target protein, herein schematically represented by VGAM1 HOST TARGET PROTEIN through VGAM3 HOST TARGET PROTEIN, all of FIG. 1.

VGAM124 RNA, herein schematically represented by VGAM1 RNA through VGAM3 RNA, binds complementarily to a host target binding site located in an untranslated region of VGAM124 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM124 host target RNA into VGAM124 host target protein, herein schematically represented by VGAM1 HOST TARGET PROTEIN through VGAM3 HOST TARGET PROTEIN, all of FIG. 1.

It is appreciated that a function of VGR383 gene, herein designated VGR GENE, is inhibition of expression of host target genes, as part of a novel viral mechanism of attacking a host. Accordingly, utilities of VGR383 gene include diagnosis, prevention and treatment of viral infection by Vaccinia Virus. Specific functions, and accordingly utilities, of VGR383 gene correlate with, and may be deduced from, the identity of the host target genes, which are inhibited by VGAM RNAs comprised in the ‘operon-like’ cluster of VGR383 gene: VGAM123 host target protein and VGAM124 host target protein, herein schematically represented by VGAM1 HOST TARGET PROTEIN through VGAM3 HOST TARGET PROTEIN. The function of these host target genes is elaborated hereinabove with reference to VGAM123 and VGAM124. FIG. 9 further provides a conceptual description of novel bioinformatically detected regulatory viral gene, referred to here as Viral Genomic Record 384(VGR384) viral gene, which encodes an ‘operon-like’ cluster of novel viral micro RNA-like genes, each of which in turn modulates expression of at least one host target gene, the function and utility of which at least one host target gene is known in the art.

VGR384 gene, herein designated VGR GENE, is a novel bioinformatically detected regulatory, non protein coding, RNA viral gene. The method by which VGR384 gene was detected is described hereinabove with reference to FIGS. 1-9.

VGR384 gene encodes VGR384 precursor RNA, herein designated VGR PRECURSOR RNA, an RNA molecule, typically several hundred nucleotides long.

VGR384 precursor RNA folds spatially, forming VGR384 folded precursor RNA, herein designated VGR FOLDED PRECURSOR RNA. It is appreciated that VGR384 folded precursor RNA comprises a plurality of what is known in the art as ‘hairpin’ structures. These ‘hairpin’ structures are due to the fact that the nucleotide sequence of VGR384 precursor RNA comprises a plurality of segments, the first half of each such segment having a nucleotide sequence which is at least a partial inversed-reversed sequence of the second half thereof, as is well known in the art.

VGR384 folded precursor RNA is naturally processed by cellular enzymatic activity into at least 2 separate VGAM precursor RNAs, VGAM125 precursor RNA and VGAM126 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM125 RNA and VGAM126 RNA, herein schematically represented by VGAM1 RNA through VGAM3 RNA, each of which VGAM RNAs corresponding to VGAM RNA of FIG. 1.

VGAM125 RNA, herein schematically represented by VGAM1 RNA through VGAM3 RNA, binds complementarily to a host target binding site located in an untranslated region of VGAM125 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM125 host target RNA into VGAM125 host target protein, herein schematically represented by VGAM1 HOST TARGET PROTEIN through VGAM3 HOST TARGET PROTEIN, all of FIG. 1.

VGAM126 RNA, herein schematically represented by VGAM1 RNA through VGAM3 RNA, binds complementarily to a host target binding site located in an untranslated region of VGAM126 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM126 host target RNA into VGAM126 host target protein, herein schematically represented by VGAM1 HOST TARGET PROTEIN through VGAM3 HOST TARGET PROTEIN, all of FIG. 1.

It is appreciated that a function of VGR384 gene, herein designated VGR GENE, is inhibition of expression of host target genes, as part of a novel viral mechanism of attacking a host. Accordingly, utilities of VGR384 gene include diagnosis, prevention and treatment of viral infection by Vaccinia Virus. Specific functions, and accordingly utilities, of VGR384 gene correlate with, and may be deduced from, the identity of the host target genes, which are inhibited by VGAM RNAs comprised in the ‘operon-like’ cluster of VGR384 gene: VGAM125 host target protein and VGAM126 host target protein, herein schematically represented by VGAM1 HOST TARGET PROTEIN through VGAM3 HOST TARGET PROTEIN. The function of these host target genes is elaborated hereinabove with reference to VGAM125 and VGAM126. FIG. 9 further provides a conceptual description of novel bioinformatically detected regulatory viral gene, referred to here as Viral Genomic Record 385(VGR385) viral gene, which encodes an ‘operon-like’ cluster of novel viral micro RNA-like genes, each of which in turn modulates expression of at least one host target gene, the function and utility of which at least one host target gene is known in the art.

VGR385 gene, herein designated VGR GENE, is a novel bioinformatically detected regulatory, non protein coding, RNA viral gene. The method by which VGR385 gene was detected is described hereinabove with reference to FIGS. 1-9.

VGR385 gene encodes VGR385 precursor RNA, herein designated VGR PRECURSOR RNA, an RNA molecule, typically several hundred nucleotides long.

VGR385 precursor RNA folds spatially, forming VGR385 folded precursor RNA, herein designated VGR FOLDED PRECURSOR RNA. It is appreciated that VGR385 folded precursor RNA comprises a plurality of what is known in the art as ‘hairpin’ structures. These ‘hairpin’ structures are due to the fact that the nucleotide sequence of VGR385 precursor RNA comprises a plurality of segments, the first half of each such segment having a nucleotide sequence which is at least a partial inversed-reversed sequence of the second half thereof, as is well known in the art.

VGR385 folded precursor RNA is naturally processed by cellular enzymatic activity into at least 2 separate VGAM precursor RNAs, VGAM127 precursor RNA and VGAM128 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM127 RNA and VGAM128 RNA, herein schematically represented by VGAM1 RNA through VGAM3 RNA, each of which VGAM RNAs corresponding to VGAM RNA of FIG. 1.

VGAM127 RNA, herein schematically represented by VGAM1 RNA through VGAM3 RNA, binds complementarily to a host target binding site located in an untranslated region of VGAM127 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM127 host target RNA into VGAM127 host target protein, herein schematically represented by VGAM1 HOST TARGET PROTEIN through VGAM3 HOST TARGET PROTEIN, all of FIG. 1.

VGAM128 RNA, herein schematically represented by VGAM1 RNA through VGAM3 RNA, binds complementarily to a host target binding site located in an untranslated region of VGAM128 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM128 host target RNA into VGAM128 host target protein, herein schematically represented by VGAM1 HOST TARGET PROTEIN through VGAM3 HOST TARGET PROTEIN, all of FIG. 1.

It is appreciated that a function of VGR385 gene, herein designated VGR GENE, is inhibition of expression of host target genes, as part of a novel viral mechanism of attacking a host. Accordingly, utilities of VGR385 gene include diagnosis, prevention and treatment of viral infection by Vaccinia Virus. Specific functions, and accordingly utilities, of VGR385 gene correlate with, and may be deduced from, the identity of the host target genes, which are inhibited by VGAM RNAs comprised in the ‘operon-like’ cluster of VGR385 gene: VGAM127 host target protein and VGAM128 host target protein, herein schematically represented by VGAM1 HOST TARGET PROTEIN through VGAM3 HOST TARGET PROTEIN. The function of these host target genes is elaborated hereinabove with reference to VGAM127 and VGAM128. FIG. 9 further provides a conceptual description of novel bioinformatically detected regulatory viral gene, referred to here as Viral Genomic Record 386(VGR386) viral gene, which encodes an ‘operon-like’ cluster of novel viral micro RNA-like genes, each of which in turn modulates expression of at least one host target gene, the function and utility of which at least one host target gene is known in the art.

VGR386 gene, herein designated VGR GENE, is a novel bioinformatically detected regulatory, non protein coding, RNA viral gene. The method by which VGR386 gene was detected is described hereinabove with reference to FIGS. 1-9.

VGR386 gene encodes VGR386 precursor RNA, herein designated VGR PRECURSOR RNA, an RNA molecule, typically several hundred nucleotides long.

VGR386 precursor RNA folds spatially, forming VGR386 folded precursor RNA, herein designated VGR FOLDED PRECURSOR RNA. It is appreciated that VGR386 folded precursor RNA comprises a plurality of what is known in the art as ‘hairpin’ structures. These ‘hairpin’ structures are due to the fact that the nucleotide sequence of VGR386 precursor RNA comprises a plurality of segments, the first half of each such segment having a nucleotide sequence which is at least a partial inversed-reversed sequence of the second half thereof, as is well known in the art.

VGR386 folded precursor RNA is naturally processed by cellular enzymatic activity into at least 3 separate VGAM precursor RNAs, VGAM130 precursor RNA, VGAM131 precursor RNA and VGAM132 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM130 RNA, VGAM131 RNA and VGAM132 RNA, herein schematically represented by VGAM1 RNA through VGAM3 RNA, each of which VGAM RNAs corresponding to VGAM RNA of FIG. 1.

VGAM130 RNA, herein schematically represented by VGAM1 RNA through VGAM3 RNA, binds complementarily to a host target binding site located in an untranslated region of VGAM130 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM130 host target RNA into VGAM130 host target protein, herein schematically represented by VGAM1 HOST TARGET PROTEIN through VGAM3 HOST TARGET PROTEIN, all of FIG. 1.

VGAM131 RNA, herein schematically represented by VGAM1 RNA through VGAM3 RNA, binds complementarily to a host target binding site located in an untranslated region of VGAM131 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM131 host target RNA into VGAM131 host target protein, herein schematically represented by VGAM1 HOST TARGET PROTEIN through VGAM3 HOST TARGET PROTEIN, all of FIG. 1.

VGAM132 RNA, herein schematically represented by VGAM1 RNA through VGAM3 RNA, binds complementarily to a host target binding site located in an untranslated region of VGAM132 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM132 host target RNA into VGAM132 host target protein, herein schematically represented by VGAM1 HOST TARGET PROTEIN through VGAM3 HOST TARGET PROTEIN, all of FIG. 1.

It is appreciated that a function of VGR386 gene, herein designated VGR GENE, is inhibition of expression of host target genes, as part of a novel viral mechanism of attacking a host. Accordingly, utilities of VGR386 gene include diagnosis, prevention and treatment of viral infection by Vaccinia Virus. Specific functions, and accordingly utilities, of VGR386 gene correlate with, and may be deduced from, the identity of the host target genes, which are inhibited by VGAM RNAs comprised in the ‘operon-like’ cluster of VGR386 gene: VGAM130 host target protein, VGAM131 host target protein and VGAM132 host target protein, herein schematically represented by VGAM1 HOST TARGET PROTEIN through VGAM3 HOST TARGET PROTEIN. The function of these host target genes is elaborated hereinabove with reference to VGAM130, VGAM131 and VGAM132. FIG. 9 further provides a conceptual description of novel bioinformatically detected regulatory viral gene, referred to here as Viral Genomic Record 387(VGR387) viral gene, which encodes an ‘operon-like’ cluster of novel viral micro RNA-like genes, each of which in turn modulates expression of at least one host target gene, the function and utility of which at least one host target gene is known in the art.

VGR387 gene, herein designated VGR GENE, is a novel bioinformatically detected regulatory, non protein coding, RNA viral gene. The method by which VGR387 gene was detected is described hereinabove with reference to FIGS. 1-9.

VGR387 gene encodes VGR387 precursor RNA, herein designated VGR PRECURSOR RNA, an RNA molecule, typically several hundred nucleotides long.

VGR387 precursor RNA folds spatially, forming VGR387 folded precursor RNA, herein designated VGR FOLDED PRECURSOR RNA. It is appreciated that VGR387 folded precursor RNA comprises a plurality of what is known in the art as ‘hairpin’ structures. These ‘hairpin’ structures are due to the fact that the nucleotide sequence of VGR387 precursor RNA comprises a plurality of segments, the first half of each such segment having a nucleotide sequence which is at least a partial inversed-reversed sequence of the second half thereof, as is well known in the art.

VGR387 folded precursor RNA is naturally processed by cellular enzymatic activity into at least 7 separate VGAM precursor RNAs, VGAM133 precursor RNA, VGAM134 precursor RNA, VGAM135 precursor RNA, VGAM136 precursor RNA, VGAM137 precursor RNA, VGAM138 precursor RNA and VGAM139 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM133 RNA, VGAM134 RNA, VGAM135 RNA, VGAM136 RNA, VGAM137 RNA, VGAM138 RNA and VGAM139 RNA, herein schematically represented by VGAM1 RNA through VGAM3 RNA, each of which VGAM RNAs corresponding to VGAM RNA of FIG. 1.

VGAM133 RNA, herein schematically represented by VGAM1 RNA through VGAM3 RNA, binds complementarily to a host target binding site located in an untranslated region of VGAM133 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM133 host target RNA into VGAM133 host target protein, herein schematically represented by VGAM1 HOST TARGET PROTEIN through VGAM3 HOST TARGET PROTEIN, all of FIG. 1.

VGAM134 RNA, herein schematically represented by VGAM1 RNA through VGAM3 RNA, binds complementarily to a host target binding site located in an untranslated region of VGAM134 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM134 host target RNA into VGAM134 host target protein, herein schematically represented by VGAM1 HOST TARGET PROTEIN through VGAM3 HOST TARGET PROTEIN, all of FIG. 1.

VGAM135 RNA, herein schematically represented by VGAM1 RNA through VGAM3 RNA, binds complementarily to a host target binding site located in an untranslated region of VGAM135 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM135 host target RNA into VGAM135 host target protein, herein schematically represented by VGAM1 HOST TARGET PROTEIN through VGAM3 HOST TARGET PROTEIN, all of FIG. 1.

VGAM136 RNA, herein schematically represented by VGAM1 RNA through VGAM3 RNA, binds complementarily to a host target binding site located in an untranslated region of VGAM136 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM136 host target RNA into VGAM136 host target protein, herein schematically represented by VGAM1 HOST TARGET PROTEIN through VGAM3 HOST TARGET PROTEIN, all of FIG. 1.

VGAM137 RNA, herein schematically represented by VGAM1 RNA through VGAM3 RNA, binds complementarily to a host target binding site located in an untranslated region of VGAM137 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM137 host target RNA into VGAM137 host target protein, herein schematically represented by VGAM1 HOST TARGET PROTEIN through VGAM3 HOST TARGET PROTEIN, all of FIG. 1.

VGAM138 RNA, herein schematically represented by VGAM1 RNA through VGAM3 RNA, binds complementarily to a host target binding site located in an untranslated region of VGAM138 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM138 host target RNA into VGAM138 host target protein, herein schematically represented by VGAM1 HOST TARGET PROTEIN through VGAM3 HOST TARGET PROTEIN, all of FIG. 1.

VGAM139 RNA, herein schematically represented by VGAM1 RNA through VGAM3 RNA, binds complementarily to a host target binding site located in an untranslated region of VGAM139 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM139 host target RNA into VGAM139 host target protein, herein schematically represented by VGAM1 HOST TARGET PROTEIN through VGAM3 HOST TARGET PROTEIN, all of FIG. 1.

It is appreciated that a function of VGR387 gene, herein designated VGR GENE, is inhibition of expression of host target genes, as part of a novel viral mechanism of attacking a host. Accordingly, utilities of VGR387 gene include diagnosis, prevention and treatment of viral infection by Vaccinia Virus. Specific functions, and accordingly utilities, of VGR387 gene correlate with, and may be deduced from, the identity of the host target genes, which are inhibited by VGAM RNAs comprised in the ‘operon-like’ cluster of VGR387 gene: VGAM133 host target protein, VGAM134 host target protein, VGAM135 host target protein, VGAM136 host target protein, VGAM137 host target protein, VGAM138 host target protein and VGAM139 host target protein, herein schematically represented by VGAM1 HOST TARGET PROTEIN through VGAM3 HOST TARGET PROTEIN. The function of these host target genes is elaborated hereinabove with reference to VGAM133, VGAM134, VGAM135, VGAM136, VGAM137, VGAM138 and VGAM139. FIG. 9 further provides a conceptual description of novel bioinformatically detected regulatory viral gene, referred to here as Viral Genomic Record 388(VGR388) viral gene, which encodes an ‘operon-like’ cluster of novel viral micro RNA-like genes, each of which in turn modulates expression of at least one host target gene, the function and utility of which at least one host target gene is known in the art.

VGR388 gene, herein designated VGR GENE, is a novel bioinformatically detected regulatory, non protein coding, RNA viral gene. The method by which VGR388 gene was detected is described hereinabove with reference to FIGS. 1-9.

VGR388 gene encodes VGR388 precursor RNA, herein designated VGR PRECURSOR RNA, an RNA molecule, typically several hundred nucleotides long.

VGR388 precursor RNA folds spatially, forming VGR388 folded precursor RNA, herein designated VGR FOLDED PRECURSOR RNA. It is appreciated that VGR388 folded precursor RNA comprises a plurality of what is known in the art as ‘hairpin’ structures. These ‘hairpin’ structures are due to the fact that the nucleotide sequence of VGR388 precursor RNA comprises a plurality of segments, the first half of each such segment having a nucleotide sequence which is at least a partial inversed-reversed sequence of the second half thereof, as is well known in the art.

VGR388 folded precursor RNA is naturally processed by cellular enzymatic activity into at least 2 separate VGAM precursor RNAs, VGAM140 precursor RNA and VGAM141 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM140 RNA and VGAM141 RNA, herein schematically represented by VGAM1 RNA through VGAM3 RNA, each of which VGAM RNAs corresponding to VGAM RNA of FIG. 1.

VGAM140 RNA, herein schematically represented by VGAM1 RNA through VGAM3 RNA, binds complementarily to a host target binding site located in an untranslated region of VGAM140 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM140 host target RNA into VGAM140 host target protein, herein schematically represented by VGAM1 HOST TARGET PROTEIN through VGAM3 HOST TARGET PROTEIN, all of FIG. 1.

VGAM141 RNA, herein schematically represented by VGAM1 RNA through VGAM3 RNA, binds complementarily to a host target binding site located in an untranslated region of VGAM141 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM141 host target RNA into VGAM141 host target protein, herein schematically represented by VGAM1 HOST TARGET PROTEIN through VGAM3 HOST TARGET PROTEIN, all of FIG. 1.

It is appreciated that a function of VGR388 gene, herein designated VGR GENE, is inhibition of expression of host target genes, as part of a novel viral mechanism of attacking a host. Accordingly, utilities of VGR388 gene include diagnosis, prevention and treatment of viral infection by Vaccinia Virus. Specific functions, and accordingly utilities, of VGR388 gene correlate with, and may be deduced from, the identity of the host target genes, which are inhibited by VGAM RNAs comprised in the ‘operon-like’ cluster of VGR388 gene: VGAM140 host target protein and VGAM141 host target protein, herein schematically represented by VGAM1 HOST TARGET PROTEIN through VGAM3 HOST TARGET PROTEIN. The function of these host target genes is elaborated hereinabove with reference to VGAM140 and VGAM141. FIG. 9 further provides a conceptual description of novel bioinformatically detected regulatory viral gene, referred to here as Viral Genomic Record 389(VGR389) viral gene, which encodes an ‘operon-like’ cluster of novel viral micro RNA-like genes, each of which in turn modulates expression of at least one host target gene, the function and utility of which at least one host target gene is known in the art.

VGR389 gene, herein designated VGR GENE, is a novel bioinformatically detected regulatory, non protein coding, RNA viral gene. The method by which VGR389 gene was detected is described hereinabove with reference to FIGS. 1-9.

VGR389 gene encodes VGR389 precursor RNA, herein designated VGR PRECURSOR RNA, an RNA molecule, typically several hundred nucleotides long.

VGR389 precursor RNA folds spatially, forming VGR389 folded precursor RNA, herein designated VGR FOLDED PRECURSOR RNA. It is appreciated that VGR389 folded precursor RNA comprises a plurality of what is known in the art as ‘hairpin’ structures. These ‘hairpin’ structures are due to the fact that the nucleotide sequence of VGR389 precursor RNA comprises a plurality of segments, the first half of each such segment having a nucleotide sequence which is at least a partial inversed-reversed sequence of the second half thereof, as is well known in the art.

VGR389 folded precursor RNA is naturally processed by cellular enzymatic activity into at least 6 separate VGAM precursor RNAs, VGAM142 precursor RNA, VGAM143 precursor RNA, VGAM144 precursor RNA, VGAM145 precursor RNA, VGAM146 precursor RNA and VGAM147 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM142 RNA, VGAM143 RNA, VGAM144 RNA, VGAM145 RNA, VGAM146 RNA and VGAM147 RNA, herein schematically represented by VGAM1 RNA through VGAM3 RNA, each of which VGAM RNAs corresponding to VGAM RNA of FIG. 1.

VGAM142 RNA, herein schematically represented by VGAM1 RNA through VGAM3 RNA, binds complementarily to a host target binding site located in an untranslated region of VGAM142 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM142 host target RNA into VGAM142 host target protein, herein schematically represented by VGAM1 HOST TARGET PROTEIN through VGAM3 HOST TARGET PROTEIN, all of FIG. 1.

VGAM143 RNA, herein schematically represented by VGAM1 RNA through VGAM3 RNA, binds complementarily to a host target binding site located in an untranslated region of VGAM143 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM143 host target RNA into VGAM143 host target protein, herein schematically represented by VGAM1 HOST TARGET PROTEIN through VGAM3 HOST TARGET PROTEIN, all of FIG. 1.

VGAM144 RNA, herein schematically represented by VGAM1 RNA through VGAM3 RNA, binds complementarily to a host target binding site located in an untranslated region of VGAM144 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM144 host target RNA into VGAM144 host target protein, herein schematically represented by VGAM1 HOST TARGET PROTEIN through VGAM3 HOST TARGET PROTEIN, all of FIG. 1.

VGAM145 RNA, herein schematically represented by VGAM1 RNA through VGAM3 RNA, binds complementarily to a host target binding site located in an untranslated region of VGAM145 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM145 host target RNA into VGAM145 host target protein, herein schematically represented by VGAM1 HOST TARGET PROTEIN through VGAM3 HOST TARGET PROTEIN, all of FIG. 1.

VGAM146 RNA, herein schematically represented by VGAM1 RNA through VGAM3 RNA, binds complementarily to a host target binding site located in an untranslated region of VGAM146 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM146 host target RNA into VGAM146 host target protein, herein schematically represented by VGAM1 HOST TARGET PROTEIN through VGAM3 HOST TARGET PROTEIN, all of FIG. 1.

VGAM147 RNA, herein schematically represented by VGAM1 RNA through VGAM3 RNA, binds complementarily to a host target binding site located in an untranslated region of VGAM147 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM147 host target RNA into VGAM147 host target protein, herein schematically represented by VGAM1 HOST TARGET PROTEIN through VGAM3 HOST TARGET PROTEIN, all of FIG. 1.

It is appreciated that a function of VGR389 gene, herein designated VGR GENE, is inhibition of expression of host target genes, as part of a novel viral mechanism of attacking a host. Accordingly, utilities of VGR389 gene include diagnosis, prevention and treatment of viral infection by Vaccinia Virus. Specific functions, and accordingly utilities, of VGR389 gene correlate with, and may be deduced from, the identity of the host target genes, which are inhibited by VGAM RNAs comprised in the ‘operon-like’ cluster of VGR389 gene: VGAM142 host target protein, VGAM143 host target protein, VGAM144 host target protein, VGAM145 host target protein, VGAM146 host target protein and VGAM147 host target protein, herein schematically represented by VGAM1 HOST TARGET PROTEIN through VGAM3 HOST TARGET PROTEIN. The function of these host target genes is elaborated hereinabove with reference to VGAM142, VGAM143, VGAM144, VGAM145, VGAM146 and VGAM147. FIG. 9 further provides a conceptual description of novel bioinformatically detected regulatory viral gene, referred to here as Viral Genomic Record 390(VGR390) viral gene, which encodes an ‘operon-like’ cluster of novel viral micro RNA-like genes, each of which in turn modulates expression of at least one host target gene, the function and utility of which at least one host target gene is known in the art.

VGR390 gene, herein designated VGR GENE, is a novel bioinformatically detected regulatory, non protein coding, RNA viral gene. The method by which VGR390 gene was detected is described hereinabove with reference to FIGS. 1-9.

VGR390 gene encodes VGR390 precursor RNA, herein designated VGR PRECURSOR RNA, an RNA molecule, typically several hundred nucleotides long.

VGR390 precursor RNA folds spatially, forming VGR390 folded precursor RNA, herein designated VGR FOLDED PRECURSOR RNA. It is appreciated that VGR390 folded precursor RNA comprises a plurality of what is known in the art as ‘hairpin’ structures. These ‘hairpin’ structures are due to the fact that the nucleotide sequence of VGR390 precursor RNA comprises a plurality of segments, the first half of each such segment having a nucleotide sequence which is at least a partial inversed-reversed sequence of the second half thereof, as is well known in the art.

VGR390 folded precursor RNA is naturally processed by cellular enzymatic activity into at least 2 separate VGAM precursor RNAs, VGAM148 precursor RNA and VGAM149 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM148 RNA and VGAM149 RNA, herein schematically represented by VGAM1 RNA through VGAM3 RNA, each of which VGAM RNAs corresponding to VGAM RNA of FIG. 1.

VGAM148 RNA, herein schematically represented by VGAM1 RNA through VGAM3 RNA, binds complementarily to a host target binding site located in an untranslated region of VGAM148 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM148 host target RNA into VGAM148 host target protein, herein schematically represented by VGAM1 HOST TARGET PROTEIN through VGAM3 HOST TARGET PROTEIN, all of FIG. 1.

VGAM149 RNA, herein schematically represented by VGAM1 RNA through VGAM3 RNA, binds complementarily to a host target binding site located in an untranslated region of VGAM149 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM149 host target RNA into VGAM149 host target protein, herein schematically represented by VGAM1 HOST TARGET PROTEIN through VGAM3 HOST TARGET PROTEIN, all of FIG. 1.

It is appreciated that a function of VGR390 gene, herein designated VGR GENE, is inhibition of expression of host target genes, as part of a novel viral mechanism of attacking a host. Accordingly, utilities of VGR390 gene include diagnosis, prevention and treatment of viral infection by Vaccinia Virus. Specific functions, and accordingly utilities, of VGR390 gene correlate with, and may be deduced from, the identity of the host target genes, which are inhibited by VGAM RNAs comprised in the ‘operon-like’ cluster of VGR390 gene: VGAM148 host target protein and VGAM149 host target protein, herein schematically represented by VGAM1 HOST TARGET PROTEIN through VGAM3 HOST TARGET PROTEIN. The function of these host target genes is elaborated hereinabove with reference to VGAM148 and VGAM149. FIG. 9 further provides a conceptual description of novel bioinformatically detected regulatory viral gene, referred to here as Viral Genomic Record 391(VGR391) viral gene, which encodes an ‘operon-like’ cluster of novel viral micro RNA-like genes, each of which in turn modulates expression of at least one host target gene, the function and utility of which at least one host target gene is known in the art.

VGR391 gene, herein designated VGR GENE, is a novel bioinformatically detected regulatory, non protein coding, RNA viral gene. The method by which VGR391 gene was detected is described hereinabove with reference to FIGS. 1-9.

VGR391 gene encodes VGR391 precursor RNA, herein designated VGR PRECURSOR RNA, an RNA molecule, typically several hundred nucleotides long.

VGR391 precursor RNA folds spatially, forming VGR391 folded precursor RNA, herein designated VGR FOLDED PRECURSOR RNA. It is appreciated that VGR391 folded precursor RNA comprises a plurality of what is known in the art as ‘hairpin’ structures. These ‘hairpin’ structures are due to the fact that the nucleotide sequence of VGR391 precursor RNA comprises a plurality of segments, the first half of each such segment having a nucleotide sequence which is at least a partial inversed-reversed sequence of the second half thereof, as is well known in the art.

VGR391 folded precursor RNA is naturally processed by cellular enzymatic activity into at least 2 separate VGAM precursor RNAs, VGAM150 precursor RNA and VGAM151 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM150 RNA and VGAM151 RNA, herein schematically represented by VGAM1 RNA through VGAM3 RNA, each of which VGAM RNAs corresponding to VGAM RNA of FIG. 1.

VGAM150 RNA, herein schematically represented by VGAM1 RNA through VGAM3 RNA, binds complementarily to a host target binding site located in an untranslated region of VGAM150 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM150 host target RNA into VGAM150 host target protein, herein schematically represented by VGAM1 HOST TARGET PROTEIN through VGAM3 HOST TARGET PROTEIN, all of FIG. 1.

VGAM151 RNA, herein schematically represented by VGAM1 RNA through VGAM3 RNA, binds complementarily to a host target binding site located in an untranslated region of VGAM151 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM151 host target RNA into VGAM151 host target protein, herein schematically represented by VGAM1 HOST TARGET PROTEIN through VGAM3 HOST TARGET PROTEIN, all of FIG. 1.

It is appreciated that a function of VGR391 gene, herein designated VGR GENE, is inhibition of expression of host target genes, as part of a novel viral mechanism of attacking a host. Accordingly, utilities of VGR391 gene include diagnosis, prevention and treatment of viral infection by Vaccinia Virus. Specific functions, and accordingly utilities, of VGR391 gene correlate with, and may be deduced from, the identity of the host target genes, which are inhibited by VGAM RNAs comprised in the ‘operon-like’ cluster of VGR391 gene: VGAM150 host target protein and VGAM151 host target protein, herein schematically represented by VGAM1 HOST TARGET PROTEIN through VGAM3 HOST TARGET PROTEIN. The function of these host target genes is elaborated hereinabove with reference to VGAM150 and VGAM151. FIG. 9 further provides a conceptual description of novel bioinformatically detected regulatory viral gene, referred to here as Viral Genomic Record 392(VGR392) viral gene, which encodes an ‘operon-like’ cluster of novel viral micro RNA-like genes, each of which in turn modulates expression of at least one host target gene, the function and utility of which at least one host target gene is known in the art.

VGR392 gene, herein designated VGR GENE, is a novel bioinformatically detected regulatory, non protein coding, RNA viral gene. The method by which VGR392 gene was detected is described hereinabove with reference to FIGS. 1-9.

VGR392 gene encodes VGR392 precursor RNA, herein designated VGR PRECURSOR RNA, an RNA molecule, typically several hundred nucleotides long.

VGR392 precursor RNA folds spatially, forming VGR392 folded precursor RNA, herein designated VGR FOLDED PRECURSOR RNA. It is appreciated that VGR392 folded precursor RNA comprises a plurality of what is known in the art as ‘hairpin’ structures. These ‘hairpin’ structures are due to the fact that the nucleotide sequence of VGR392 precursor RNA comprises a plurality of segments, the first half of each such segment having a nucleotide sequence which is at least a partial inversed-reversed sequence of the second half thereof, as is well known in the art.

VGR392 folded precursor RNA is naturally processed by cellular enzymatic activity into at least 4 separate VGAM precursor RNAs, VGAM154 precursor RNA, VGAM155 precursor RNA, VGAM156 precursor RNA and VGAM157 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM154 RNA, VGAM155 RNA, VGAM156 RNA and VGAM157 RNA, herein schematically represented by VGAM1 RNA through VGAM3 RNA, each of which VGAM RNAs corresponding to VGAM RNA of FIG. 1.

VGAM154 RNA, herein schematically represented by VGAM1 RNA through VGAM3 RNA, binds complementarily to a host target binding site located in an untranslated region of VGAM154 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM154 host target RNA into VGAM154 host target protein, herein schematically represented by VGAM1 HOST TARGET PROTEIN through VGAM3 HOST TARGET PROTEIN, all of FIG. 1.

VGAM155 RNA, herein schematically represented by VGAM1 RNA through VGAM3 RNA, binds complementarily to a host target binding site located in an untranslated region of VGAM155 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM155 host target RNA into VGAM155 host target protein, herein schematically represented by VGAM1 HOST TARGET PROTEIN through VGAM3 HOST TARGET PROTEIN, all of FIG. 1.

VGAM156 RNA, herein schematically represented by VGAM1 RNA through VGAM3 RNA, binds complementarily to a host target binding site located in an untranslated region of VGAM156 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM156 host target RNA into VGAM156 host target protein, herein schematically represented by VGAM1 HOST TARGET PROTEIN through VGAM3 HOST TARGET PROTEIN, all of FIG. 1.

VGAM157 RNA, herein schematically represented by VGAM1 RNA through VGAM3 RNA, binds complementarily to a host target binding site located in an untranslated region of VGAM157 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM157 host target RNA into VGAM157 host target protein, herein schematically represented by VGAM1 HOST TARGET PROTEIN through VGAM3 HOST TARGET PROTEIN, all of FIG. 1.

It is appreciated that a function of VGR392 gene, herein designated VGR GENE, is inhibition of expression of host target genes, as part of a novel viral mechanism of attacking a host. Accordingly, utilities of VGR392 gene include diagnosis, prevention and treatment of viral infection by Vaccinia Virus. Specific functions, and accordingly utilities, of VGR392 gene correlate with, and may be deduced from, the identity of the host target genes, which are inhibited by VGAM RNAs comprised in the ‘operon-like’ cluster of VGR392 gene: VGAM154 host target protein, VGAM155 host target protein, VGAM156 host target protein and VGAM157 host target protein, herein schematically represented by VGAM1 HOST TARGET PROTEIN through VGAM3 HOST TARGET PROTEIN. The function of these host target genes is elaborated hereinabove with reference to VGAM154, VGAM155, VGAM156 and VGAM157. FIG. 9 further provides a conceptual description of novel bioinformatically detected regulatory viral gene, referred to here as Viral Genomic Record 393(VGR393) viral gene, which encodes an ‘operon-like’ cluster of novel viral micro RNA-like genes, each of which in turn modulates expression of at least one host target gene, the function and utility of which at least one host target gene is known in the art.

VGR393 gene, herein designated VGR GENE, is a novel bioinformatically detected regulatory, non protein coding, RNA viral gene. The method by which VGR393 gene was detected is described hereinabove with reference to FIGS. 1-9.

VGR393 gene encodes VGR393 precursor RNA, herein designated VGR PRECURSOR RNA, an RNA molecule, typically several hundred nucleotides long.

VGR393 precursor RNA folds spatially, forming VGR393 folded precursor RNA, herein designated VGR FOLDED PRECURSOR RNA. It is appreciated that VGR393 folded precursor RNA comprises a plurality of what is known in the art as ‘hairpin’ structures. These ‘hairpin’ structures are due to the fact that the nucleotide sequence of VGR393 precursor RNA comprises a plurality of segments, the first half of each such segment having a nucleotide sequence which is at least a partial inversed-reversed sequence of the second half thereof, as is well known in the art.

VGR393 folded precursor RNA is naturally processed by cellular enzymatic activity into at least 5 separate VGAM precursor RNAs, VGAM158 precursor RNA, VGAM159 precursor RNA, VGAM160 precursor RNA, VGAM161 precursor RNA and VGAM162 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM158 RNA, VGAM159 RNA, VGAM160 RNA, VGAM161 RNA and VGAM162 RNA, herein schematically represented by VGAM1 RNA through VGAM3 RNA, each of which VGAM RNAs corresponding to VGAM RNA of FIG. 1.

VGAM158 RNA, herein schematically represented by VGAM1 RNA through VGAM3 RNA, binds complementarily to a host target binding site located in an untranslated region of VGAM158 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM158 host target RNA into VGAM158 host target protein, herein schematically represented by VGAM1 HOST TARGET PROTEIN through VGAM3 HOST TARGET PROTEIN, all of FIG. 1.

VGAM159 RNA, herein schematically represented by VGAM1 RNA through VGAM3 RNA, binds complementarily to a host target binding site located in an untranslated region of VGAM159 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM159 host target RNA into VGAM159 host target protein, herein schematically represented by VGAM1 HOST TARGET PROTEIN through VGAM3 HOST TARGET PROTEIN, all of FIG. 1.

VGAM160 RNA, herein schematically represented by VGAM1 RNA through VGAM3 RNA, binds complementarily to a host target binding site located in an untranslated region of VGAM160 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM160 host target RNA into VGAM160 host target protein, herein schematically represented by VGAM1 HOST TARGET PROTEIN through VGAM3 HOST TARGET PROTEIN, all of FIG. 1.

VGAM161 RNA, herein schematically represented by VGAM1 RNA through VGAM3 RNA, binds complementarily to a host target binding site located in an untranslated region of VGAM161 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM161 host target RNA into VGAM161 host target protein, herein schematically represented by VGAM1 HOST TARGET PROTEIN through VGAM3 HOST TARGET PROTEIN, all of FIG. 1.

VGAM162 RNA, herein schematically represented by VGAM1 RNA through VGAM3 RNA, binds complementarily to a host target binding site located in an untranslated region of VGAM162 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM162 host target RNA into VGAM162 host target protein, herein schematically represented by VGAM1 HOST TARGET PROTEIN through VGAM3 HOST TARGET PROTEIN, all of FIG. 1.

It is appreciated that a function of VGR393 gene, herein designated VGR GENE, is inhibition of expression of host target genes, as part of a novel viral mechanism of attacking a host. Accordingly, utilities of VGR393 gene include diagnosis, prevention and treatment of viral infection by Vaccinia Virus. Specific functions, and accordingly utilities, of VGR393 gene correlate with, and may be deduced from, the identity of the host target genes, which are inhibited by VGAM RNAs comprised in the ‘operon-like’ cluster of VGR393 gene: VGAM158 host target protein, VGAM159 host target protein, VGAM160 host target protein, VGAM161 host target protein and VGAM162 host target protein, herein schematically represented by VGAM1 HOST TARGET PROTEIN through VGAM3 HOST TARGET PROTEIN. The function of these host target genes is elaborated hereinabove with reference to VGAM158, VGAM159, VGAM160, VGAM161 and VGAM162. FIG. 9 further provides a conceptual description of novel bioinformatically detected regulatory viral gene, referred to here as Viral Genomic Record 394(VGR394) viral gene, which encodes an ‘operon-like’ cluster of novel viral micro RNA-like genes, each of which in turn modulates expression of at least one host target gene, the function and utility of which at least one host target gene is known in the art.

VGR394 gene, herein designated VGR GENE, is a novel bioinformatically detected regulatory, non protein coding, RNA viral gene. The method by which VGR394 gene was detected is described hereinabove with reference to FIGS. 1-9.

VGR394 gene encodes VGR394 precursor RNA, herein designated VGR PRECURSOR RNA, an RNA molecule, typically several hundred nucleotides long.

VGR394 precursor RNA folds spatially, forming VGR394 folded precursor RNA, herein designated VGR FOLDED PRECURSOR RNA. It is appreciated that VGR394 folded precursor RNA comprises a plurality of what is known in the art as ‘hairpin’ structures. These ‘hairpin’ structures are due to the fact that the nucleotide sequence of VGR394 precursor RNA comprises a plurality of segments, the first half of each such segment having a nucleotide sequence which is at least a partial inversed-reversed sequence of the second half thereof, as is well known in the art.

VGR394 folded precursor RNA is naturally processed by cellular enzymatic activity into at least 6 separate VGAM precursor RNAs, VGAM163 precursor RNA, VGAM164 precursor RNA, VGAM165 precursor RNA, VGAM166 precursor RNA, VGAM167 precursor RNA and VGAM168 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM163 RNA, VGAM164 RNA, VGAM165 RNA, VGAM166 RNA, VGAM167 RNA and VGAM168 RNA, herein schematically represented by VGAM1 RNA through VGAM3 RNA, each of which VGAM RNAs corresponding to VGAM RNA of FIG. 1.

VGAM163 RNA, herein schematically represented by VGAM1 RNA through VGAM3 RNA, binds complementarily to a host target binding site located in an untranslated region of VGAM163 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM163 host target RNA into VGAM163 host target protein, herein schematically represented by VGAM1 HOST TARGET PROTEIN through VGAM3 HOST TARGET PROTEIN, all of FIG. 1.

VGAM164 RNA, herein schematically represented by VGAM1 RNA through VGAM3 RNA, binds complementarily to a host target binding site located in an untranslated region of VGAM164 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM164 host target RNA into VGAM164 host target protein, herein schematically represented by VGAM1 HOST TARGET PROTEIN through VGAM3 HOST TARGET PROTEIN, all of FIG. 1.

VGAM165 RNA, herein schematically represented by VGAM1 RNA through VGAM3 RNA, binds complementarily to a host target binding site located in an untranslated region of VGAM165 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM165 host target RNA into VGAM165 host target protein, herein schematically represented by VGAM1 HOST TARGET PROTEIN through VGAM3 HOST TARGET PROTEIN, all of FIG. 1.

VGAM166 RNA, herein schematically represented by VGAM1 RNA through VGAM3 RNA, binds complementarily to a host target binding site located in an untranslated region of VGAM166 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM166 host target RNA into VGAM166 host target protein, herein schematically represented by VGAM1 HOST TARGET PROTEIN through VGAM3 HOST TARGET PROTEIN, all of FIG. 1.

VGAM167 RNA, herein schematically represented by VGAM1 RNA through VGAM3 RNA, binds complementarily to a host target binding site located in an untranslated region of VGAM167 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM167 host target RNA into VGAM167 host target protein, herein schematically represented by VGAM1 HOST TARGET PROTEIN through VGAM3 HOST TARGET PROTEIN, all of FIG. 1.

VGAM168 RNA, herein schematically represented by VGAM1 RNA through VGAM3 RNA, binds complementarily to a host target binding site located in an untranslated region of VGAM168 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM168 host target RNA into VGAM168 host target protein, herein schematically represented by VGAM1 HOST TARGET PROTEIN through VGAM3 HOST TARGET PROTEIN, all of FIG. 1.

It is appreciated that a function of VGR394 gene, herein designated VGR GENE, is inhibition of expression of host target genes, as part of a novel viral mechanism of attacking a host. Accordingly, utilities of VGR394 gene include diagnosis, prevention and treatment of viral infection by Vaccinia Virus. Specific functions, and accordingly utilities, of VGR394 gene correlate with, and may be deduced from, the identity of the host target genes, which are inhibited by VGAM RNAs comprised in the ‘operon-like’ cluster of VGR394 gene: VGAM163 host target protein, VGAM164 host target protein, VGAM165 host target protein, VGAM166 host target protein, VGAM167 host target protein and VGAM168 host target protein, herein schematically represented by VGAM1 HOST TARGET PROTEIN through VGAM3 HOST TARGET PROTEIN. The function of these host target genes is elaborated hereinabove with reference to VGAM163, VGAM164, VGAM165, VGAM166, VGAM167 and VGAM168. FIG. 9 further provides a conceptual description of novel bioinformatically detected regulatory viral gene, referred to here as Viral Genomic Record 395(VGR395) viral gene, which encodes an ‘operon-like’ cluster of novel viral micro RNA-like genes, each of which in turn modulates expression of at least one host target gene, the function and utility of which at least one host target gene is known in the art.

VGR395 gene, herein designated VGR GENE, is a novel bioinformatically detected regulatory, non protein coding, RNA viral gene. The method by which VGR395 gene was detected is described hereinabove with reference to FIGS. 1-9.

VGR395 gene encodes VGR395 precursor RNA, herein designated VGR PRECURSOR RNA, an RNA molecule, typically several hundred nucleotides long.

VGR395 precursor RNA folds spatially, forming VGR395 folded precursor RNA, herein designated VGR FOLDED PRECURSOR RNA. It is appreciated that VGR395 folded precursor RNA comprises a plurality of what is known in the art as ‘hairpin’ structures. These ‘hairpin’ structures are due to the fact that the nucleotide sequence of VGR395 precursor RNA comprises a plurality of segments, the first half of each such segment having a nucleotide sequence which is at least a partial inversed-reversed sequence of the second half thereof, as is well known in the art.

VGR395 folded precursor RNA is naturally processed by cellular enzymatic activity into at least 2 separate VGAM precursor RNAs, VGAM169 precursor RNA and VGAM170 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM169 RNA and VGAM170 RNA, herein schematically represented by VGAM1 RNA through VGAM3 RNA, each of which VGAM RNAs corresponding to VGAM RNA of FIG. 1.

VGAM169 RNA, herein schematically represented by VGAM1 RNA through VGAM3 RNA, binds complementarily to a host target binding site located in an untranslated region of VGAM169 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM169 host target RNA into VGAM169 host target protein, herein schematically represented by VGAM1 HOST TARGET PROTEIN through VGAM3 HOST TARGET PROTEIN, all of FIG. 1.

VGAM170 RNA, herein schematically represented by VGAM1 RNA through VGAM3 RNA, binds complementarily to a host target binding site located in an untranslated region of VGAM170 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM170 host target RNA into VGAM170 host target protein, herein schematically represented by VGAM1 HOST TARGET PROTEIN through VGAM3 HOST TARGET PROTEIN, all of FIG. 1.

It is appreciated that a function of VGR395 gene, herein designated VGR GENE, is inhibition of expression of host target genes, as part of a novel viral mechanism of attacking a host. Accordingly, utilities of VGR395 gene include diagnosis, prevention and treatment of viral infection by Vaccinia Virus. Specific functions, and accordingly utilities, of VGR395 gene correlate with, and may be deduced from, the identity of the host target genes, which are inhibited by VGAM RNAs comprised in the ‘operon-like’ cluster of VGR395 gene: VGAM169 host target protein and VGAM170 host target protein, herein schematically represented by VGAM1 HOST TARGET PROTEIN through VGAM3 HOST TARGET PROTEIN. The function of these host target genes is elaborated hereinabove with reference to VGAM169 and VGAM170. FIG. 9 further provides a conceptual description of novel bioinformatically detected regulatory viral gene, referred to here as Viral Genomic Record 396(VGR396) viral gene, which encodes an ‘operon-like’ cluster of novel viral micro RNA-like genes, each of which in turn modulates expression of at least one host target gene, the function and utility of which at least one host target gene is known in the art.

VGR396 gene, herein designated VGR GENE, is a novel bioinformatically detected regulatory, non protein coding, RNA viral gene. The method by which VGR396 gene was detected is described hereinabove with reference to FIGS. 1-9.

VGR396 gene encodes VGR396 precursor RNA, herein designated VGR PRECURSOR RNA, an RNA molecule, typically several hundred nucleotides long.

VGR396 precursor RNA folds spatially, forming VGR396 folded precursor RNA, herein designated VGR FOLDED PRECURSOR RNA. It is appreciated that VGR396 folded precursor RNA comprises a plurality of what is known in the art as ‘hairpin’ structures. These ‘hairpin’ structures are due to the fact that the nucleotide sequence of VGR396 precursor RNA comprises a plurality of segments, the first half of each such segment having a nucleotide sequence which is at least a partial inversed-reversed sequence of the second half thereof, as is well known in the art.

VGR396 folded precursor RNA is naturally processed by cellular enzymatic activity into at least 2 separate VGAM precursor RNAs, VGAM171 precursor RNA and VGAM172 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM171 RNA and VGAM172 RNA, herein schematically represented by VGAM1 RNA through VGAM3 RNA, each of which VGAM RNAs corresponding to VGAM RNA of FIG. 1.

VGAM171 RNA, herein schematically represented by VGAM1 RNA through VGAM3 RNA, binds complementarily to a host target binding site located in an untranslated region of VGAM171 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM171 host target RNA into VGAM171 host target protein, herein schematically represented by VGAM1 HOST TARGET PROTEIN through VGAM3 HOST TARGET PROTEIN, all of FIG. 1.

VGAM172 RNA, herein schematically represented by VGAM1 RNA through VGAM3 RNA, binds complementarily to a host target binding site located in an untranslated region of VGAM172 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM172 host target RNA into VGAM172 host target protein, herein schematically represented by VGAM1 HOST TARGET PROTEIN through VGAM3 HOST TARGET PROTEIN, all of FIG. 1.

It is appreciated that a function of VGR396 gene, herein designated VGR GENE, is inhibition of expression of host target genes, as part of a novel viral mechanism of attacking a host. Accordingly, utilities of VGR396 gene include diagnosis, prevention and treatment of viral infection by Vaccinia Virus. Specific functions, and accordingly utilities, of VGR396 gene correlate with, and may be deduced from, the identity of the host target genes, which are inhibited by VGAM RNAs comprised in the ‘operon-like’ cluster of VGR396 gene: VGAM171 host target protein and VGAM172 host target protein, herein schematically represented by VGAM1 HOST TARGET PROTEIN through VGAM3 HOST TARGET PROTEIN. The function of these host target genes is elaborated hereinabove with reference to VGAM171 and VGAM172. FIG. 9 further provides a conceptual description of novel bioinformatically detected regulatory viral gene, referred to here as Viral Genomic Record 397(VGR397) viral gene, which encodes an ‘operon-like’ cluster of novel viral micro RNA-like genes, each of which in turn modulates expression of at least one host target gene, the function and utility of which at least one host target gene is known in the art.

VGR397 gene, herein designated VGR GENE, is a novel bioinformatically detected regulatory, non protein coding, RNA viral gene. The method by which VGR397 gene was detected is described hereinabove with reference to FIGS. 1-9.

VGR397 gene encodes VGR397 precursor RNA, herein designated VGR PRECURSOR RNA, an RNA molecule, typically several hundred nucleotides long.

VGR397 precursor RNA folds spatially, forming VGR397 folded precursor RNA, herein designated VGR FOLDED PRECURSOR RNA. It is appreciated that VGR397 folded precursor RNA comprises a plurality of what is known in the art as ‘hairpin’ structures. These ‘hairpin’ structures are due to the fact that the nucleotide sequence of VGR397 precursor RNA comprises a plurality of segments, the first half of each such segment having a nucleotide sequence which is at least a partial inversed-reversed sequence of the second half thereof, as is well known in the art.

VGR397 folded precursor RNA is naturally processed by cellular enzymatic activity into at least 2 separate VGAM precursor RNAs, VGAM173 precursor RNA and VGAM174 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM173 RNA and VGAM174 RNA, herein schematically represented by VGAM1 RNA through VGAM3 RNA, each of which VGAM RNAs corresponding to VGAM RNA of FIG. 1.

VGAM173 RNA, herein schematically represented by VGAM1 RNA through VGAM3 RNA, binds complementarily to a host target binding site located in an untranslated region of VGAM173 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM173 host target RNA into VGAM173 host target protein, herein schematically represented by VGAM1 HOST TARGET PROTEIN through VGAM3 HOST TARGET PROTEIN, all of FIG. 1.

VGAM174 RNA, herein schematically represented by VGAM1 RNA through VGAM3 RNA, binds complementarily to a host target binding site located in an untranslated region of VGAM174 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM174 host target RNA into VGAM174 host target protein, herein schematically represented by VGAM1 HOST TARGET PROTEIN through VGAM3 HOST TARGET PROTEIN, all of FIG. 1.

It is appreciated that a function of VGR397 gene, herein designated VGR GENE, is inhibition of expression of host target genes, as part of a novel viral mechanism of attacking a host. Accordingly, utilities of VGR397 gene include diagnosis, prevention and treatment of viral infection by Vaccinia Virus. Specific functions, and accordingly utilities, of VGR397 gene correlate with, and may be deduced from, the identity of the host target genes, which are inhibited by VGAM RNAs comprised in the ‘operon-like’ cluster of VGR397 gene: VGAM173 host target protein and VGAM174 host target protein, herein schematically represented by VGAM1 HOST TARGET PROTEIN through VGAM3 HOST TARGET PROTEIN. The function of these host target genes is elaborated hereinabove with reference to VGAM173 and VGAM174. FIG. 9 further provides a conceptual description of novel bioinformatically detected regulatory viral gene, referred to here as Viral Genomic Record 398(VGR398) viral gene, which encodes an ‘operon-like’ cluster of novel viral micro RNA-like genes, each of which in turn modulates expression of at least one host target gene, the function and utility of which at least one host target gene is known in the art.

VGR398 gene, herein designated VGR GENE, is a novel bioinformatically detected regulatory, non protein coding, RNA viral gene. The method by which VGR398 gene was detected is described hereinabove with reference to FIGS. 1-9.

VGR398 gene encodes VGR398 precursor RNA, herein designated VGR PRECURSOR RNA, an RNA molecule, typically several hundred nucleotides long.

VGR398 precursor RNA folds spatially, forming VGR398 folded precursor RNA, herein designated VGR FOLDED PRECURSOR RNA. It is appreciated that VGR398 folded precursor RNA comprises a plurality of what is known in the art as ‘hairpin’ structures. These ‘hairpin’ structures are due to the fact that the nucleotide sequence of VGR398 precursor RNA comprises a plurality of segments, the first half of each such segment having a nucleotide sequence which is at least a partial inversed-reversed sequence of the second half thereof, as is well known in the art.

VGR398 folded precursor RNA is naturally processed by cellular enzymatic activity into at least 2 separate VGAM precursor RNAs, VGAM175 precursor RNA and VGAM176 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM175 RNA and VGAM176 RNA, herein schematically represented by VGAM1 RNA through VGAM3 RNA, each of which VGAM RNAs corresponding to VGAM RNA of FIG. 1.

VGAM175 RNA, herein schematically represented by VGAM1 RNA through VGAM3 RNA, binds complementarily to a host target binding site located in an untranslated region of VGAM175 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM175 host target RNA into VGAM175 host target protein, herein schematically represented by VGAM1 HOST TARGET PROTEIN through VGAM3 HOST TARGET PROTEIN, all of FIG. 1.

VGAM176 RNA, herein schematically represented by VGAM1 RNA through VGAM3 RNA, binds complementarily to a host target binding site located in an untranslated region of VGAM176 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM176 host target RNA into VGAM176 host target protein, herein schematically represented by VGAM1 HOST TARGET PROTEIN through VGAM3 HOST TARGET PROTEIN, all of FIG. 1.

It is appreciated that a function of VGR398 gene, herein designated VGR GENE, is inhibition of expression of host target genes, as part of a novel viral mechanism of attacking a host. Accordingly, utilities of VGR398 gene include diagnosis, prevention and treatment of viral infection by Vaccinia Virus. Specific functions, and accordingly utilities, of VGR398 gene correlate with, and may be deduced from, the identity of the host target genes, which are inhibited by VGAM RNAs comprised in the ‘operon-like’ cluster of VGR398 gene: VGAM175 host target protein and VGAM176 host target protein, herein schematically represented by VGAM1 HOST TARGET PROTEIN through VGAM3 HOST TARGET PROTEIN. The function of these host target genes is elaborated hereinabove with reference to VGAM175 and VGAM176. FIG. 9 further provides a conceptual description of novel bioinformatically detected regulatory viral gene, referred to here as Viral Genomic Record 399(VGR399) viral gene, which encodes an ‘operon-like’ cluster of novel viral micro RNA-like genes, each of which in turn modulates expression of at least one host target gene, the function and utility of which at least one host target gene is known in the art.

VGR399 gene, herein designated VGR GENE, is a novel bioinformatically detected regulatory, non protein coding, RNA viral gene. The method by which VGR399 gene was detected is described hereinabove with reference to FIGS. 1-9.

VGR399 gene encodes VGR399 precursor RNA, herein designated VGR PRECURSOR RNA, an RNA molecule, typically several hundred nucleotides long.

VGR399 precursor RNA folds spatially, forming VGR399 folded precursor RNA, herein designated VGR FOLDED PRECURSOR RNA. It is appreciated that VGR399 folded precursor RNA comprises a plurality of what is known in the art as ‘hairpin’ structures. These ‘hairpin’ structures are due to the fact that the nucleotide sequence of VGR399 precursor RNA comprises a plurality of segments, the first half of each such segment having a nucleotide sequence which is at least a partial inversed-reversed sequence of the second half thereof, as is well known in the art.

VGR399 folded precursor RNA is naturally processed by cellular enzymatic activity into at least 4 separate VGAM precursor RNAs, VGAM177 precursor RNA, VGAM178 precursor RNA, VGAM179 precursor RNA and VGAM180 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM177 RNA, VGAM178 RNA, VGAM179 RNA and VGAM180 RNA, herein schematically represented by VGAM1 RNA through VGAM3 RNA, each of which VGAM RNAs corresponding to VGAM RNA of FIG. 1.

VGAM177 RNA, herein schematically represented by VGAM1 RNA through VGAM3 RNA, binds complementarily to a host target binding site located in an untranslated region of VGAM177 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM177 host target RNA into VGAM177 host target protein, herein schematically represented by VGAM1 HOST TARGET PROTEIN through VGAM3 HOST TARGET PROTEIN, all of FIG. 1.

VGAM178 RNA, herein schematically represented by VGAM1 RNA through VGAM3 RNA, binds complementarily to a host target binding site located in an untranslated region of VGAM178 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM178 host target RNA into VGAM178 host target protein, herein schematically represented by VGAM1 HOST TARGET PROTEIN through VGAM3 HOST TARGET PROTEIN, all of FIG. 1.

VGAM179 RNA, herein schematically represented by VGAM1 RNA through VGAM3 RNA, binds complementarily to a host target binding site located in an untranslated region of VGAM179 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM179 host target RNA into VGAM179 host target protein, herein schematically represented by VGAM1 HOST TARGET PROTEIN through VGAM3 HOST TARGET PROTEIN, all of FIG. 1.

VGAM180 RNA, herein schematically represented by VGAM1 RNA through VGAM3 RNA, binds complementarily to a host target binding site located in an untranslated region of VGAM180 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM180 host target RNA into VGAM180 host target protein, herein schematically represented by VGAM1 HOST TARGET PROTEIN through VGAM3 HOST TARGET PROTEIN, all of FIG. 1.

It is appreciated that a function of VGR399 gene, herein designated VGR GENE, is inhibition of expression of host target genes, as part of a novel viral mechanism of attacking a host. Accordingly, utilities of VGR399 gene include diagnosis, prevention and treatment of viral infection by Vaccinia Virus. Specific functions, and accordingly utilities, of VGR399 gene correlate with, and may be deduced from, the identity of the host target genes, which are inhibited by VGAM RNAs comprised in the ‘operon-like’ cluster of VGR399 gene: VGAM177 host target protein, VGAM178 host target protein, VGAM179 host target protein and VGAM180 host target protein, herein schematically represented by VGAM1 HOST TARGET PROTEIN through VGAM3 HOST TARGET PROTEIN. The function of these host target genes is elaborated hereinabove with reference to VGAM177, VGAM178, VGAM179 and VGAM180. FIG. 9 further provides a conceptual description of novel bioinformatically detected regulatory viral gene, referred to here as Viral Genomic Record 400(VGR400) viral gene, which encodes an ‘operon-like’ cluster of novel viral micro RNA-like genes, each of which in turn modulates expression of at least one host target gene, the function and utility of which at least one host target gene is known in the art.

VGR400 gene, herein designated VGR GENE, is a novel bioinformatically detected regulatory, non protein coding, RNA viral gene. The method by which VGR400 gene was detected is described hereinabove with reference to FIGS. 1-9.

VGR400 gene encodes VGR400 precursor RNA, herein designated VGR PRECURSOR RNA, an RNA molecule, typically several hundred nucleotides long.

VGR400 precursor RNA folds spatially, forming VGR400 folded precursor RNA, herein designated VGR FOLDED PRECURSOR RNA. It is appreciated that VGR400 folded precursor RNA comprises a plurality of what is known in the art as ‘hairpin’ structures. These ‘hairpin’ structures are due to the fact that the nucleotide sequence of VGR400 precursor RNA comprises a plurality of segments, the first half of each such segment having a nucleotide sequence which is at least a partial inversed-reversed sequence of the second half thereof, as is well known in the art.

VGR400 folded precursor RNA is naturally processed by cellular enzymatic activity into at least 7 separate VGAM precursor RNAs, VGAM181 precursor RNA, VGAM182 precursor RNA, VGAM183 precursor RNA, VGAM184 precursor RNA, VGAM185 precursor RNA, VGAM186 precursor RNA and VGAM187 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM181 RNA, VGAM182 RNA, VGAM183 RNA, VGAM184 RNA, VGAM185 RNA, VGAM186 RNA and VGAM187 RNA, herein schematically represented by VGAM1 RNA through VGAM3 RNA, each of which VGAM RNAs corresponding to VGAM RNA of FIG. 1.

VGAM181 RNA, herein schematically represented by VGAM1 RNA through VGAM3 RNA, binds complementarily to a host target binding site located in an untranslated region of VGAM181 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM181 host target RNA into VGAM181 host target protein, herein schematically represented by VGAM1 HOST TARGET PROTEIN through VGAM3 HOST TARGET PROTEIN, all of FIG. 1.

VGAM182 RNA, herein schematically represented by VGAM1 RNA through VGAM3 RNA, binds complementarily to a host target binding site located in an untranslated region of VGAM182 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM182 host target RNA into VGAM182 host target protein, herein schematically represented by VGAM1 HOST TARGET PROTEIN through VGAM3 HOST TARGET PROTEIN, all of FIG. 1.

VGAM183 RNA, herein schematically represented by VGAM1 RNA through VGAM3 RNA, binds complementarily to a host target binding site located in an untranslated region of VGAM183 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM183 host target RNA into VGAM183 host target protein, herein schematically represented by VGAM1 HOST TARGET PROTEIN through VGAM3 HOST TARGET PROTEIN, all of FIG. 1.

VGAM184 RNA, herein schematically represented by VGAM1 RNA through VGAM3 RNA, binds complementarily to a host target binding site located in an untranslated region of VGAM184 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM184 host target RNA into VGAM184 host target protein, herein schematically represented by VGAM1 HOST TARGET PROTEIN through VGAM3 HOST TARGET PROTEIN, all of FIG. 1.

VGAM185 RNA, herein schematically represented by VGAM1 RNA through VGAM3 RNA, binds complementarily to a host target binding site located in an untranslated region of VGAM185 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM185 host target RNA into VGAM185 host target protein, herein schematically represented by VGAM1 HOST TARGET PROTEIN through VGAM3 HOST TARGET PROTEIN, all of FIG. 1.

VGAM186 RNA, herein schematically represented by VGAM1 RNA through VGAM3 RNA, binds complementarily to a host target binding site located in an untranslated region of VGAM186 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM186 host target RNA into VGAM186 host target protein, herein schematically represented by VGAM1 HOST TARGET PROTEIN through VGAM3 HOST TARGET PROTEIN, all of FIG. 1.

VGAM187 RNA, herein schematically represented by VGAM1 RNA through VGAM3 RNA, binds complementarily to a host target binding site located in an untranslated region of VGAM187 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM187 host target RNA into VGAM187 host target protein, herein schematically represented by VGAM1 HOST TARGET PROTEIN through VGAM3 HOST TARGET PROTEIN, all of FIG. 1.

It is appreciated that a function of VGR400 gene, herein designated VGR GENE, is inhibition of expression of host target genes, as part of a novel viral mechanism of attacking a host. Accordingly, utilities of VGR400 gene include diagnosis, prevention and treatment of viral infection by Vaccinia Virus. Specific functions, and accordingly utilities, of VGR400 gene correlate with, and may be deduced from, the identity of the host target genes, which are inhibited by VGAM RNAs comprised in the ‘operon-like’ cluster of VGR400 gene: VGAM181 host target protein, VGAM182 host target protein, VGAM183 host target protein, VGAM184 host target protein, VGAM185 host target protein, VGAM186 host target protein and VGAM187 host target protein, herein schematically represented by VGAM1 HOST TARGET PROTEIN through VGAM3 HOST TARGET PROTEIN. The function of these host target genes is elaborated hereinabove with reference to VGAM181, VGAM182, VGAM183, VGAM184, VGAM185, VGAM186 and VGAM187. FIG. 9 further provides a conceptual description of novel bioinformatically detected regulatory viral gene, referred to here as Viral Genomic Record 401(VGR401) viral gene, which encodes an ‘operon-like’ cluster of novel viral micro RNA-like genes, each of which in turn modulates expression of at least one host target gene, the function and utility of which at least one host target gene is known in the art.

VGR401 gene, herein designated VGR GENE, is a novel bioinformatically detected regulatory, non protein coding, RNA viral gene. The method by which VGR401 gene was detected is described hereinabove with reference to FIGS. 1-9.

VGR401 gene encodes VGR401 precursor RNA, herein designated VGR PRECURSOR RNA, an RNA molecule, typically several hundred nucleotides long.

VGR401 precursor RNA folds spatially, forming VGR401 folded precursor RNA, herein designated VGR FOLDED PRECURSOR RNA. It is appreciated that VGR401 folded precursor RNA comprises a plurality of what is known in the art as ‘hairpin’ structures. These ‘hairpin’ structures are due to the fact that the nucleotide sequence of VGR401 precursor RNA comprises a plurality of segments, the first half of each such segment having a nucleotide sequence which is at least a partial inversed-reversed sequence of the second half thereof, as is well known in the art.

VGR401 folded precursor RNA is naturally processed by cellular enzymatic activity into at least 2 separate VGAM precursor RNAs, VGAM188 precursor RNA and VGAM189 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM188 RNA and VGAM189 RNA, herein schematically represented by VGAM1 RNA through VGAM3 RNA, each of which VGAM RNAs corresponding to VGAM RNA of FIG. 1.

VGAM188 RNA, herein schematically represented by VGAM1 RNA through VGAM3 RNA, binds complementarily to a host target binding site located in an untranslated region of VGAM188 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM188 host target RNA into VGAM188 host target protein, herein schematically represented by VGAM1 HOST TARGET PROTEIN through VGAM3 HOST TARGET PROTEIN, all of FIG. 1.

VGAM189 RNA, herein schematically represented by VGAM1 RNA through VGAM3 RNA, binds complementarily to a host target binding site located in an untranslated region of VGAM189 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM189 host target RNA into VGAM189 host target protein, herein schematically represented by VGAM1 HOST TARGET PROTEIN through VGAM3 HOST TARGET PROTEIN, all of FIG. 1.

It is appreciated that a function of VGR401 gene, herein designated VGR GENE, is inhibition of expression of host target genes, as part of a novel viral mechanism of attacking a host. Accordingly, utilities of VGR401 gene include diagnosis, prevention and treatment of viral infection by Vaccinia Virus. Specific functions, and accordingly utilities, of VGR401 gene correlate with, and may be deduced from, the identity of the host target genes, which are inhibited by VGAM RNAs comprised in the ‘operon-like’ cluster of VGR401 gene: VGAM188 host target protein and VGAM189 host target protein, herein schematically represented by VGAM1 HOST TARGET PROTEIN through VGAM3 HOST TARGET PROTEIN. The function of these host target genes is elaborated hereinabove with reference to VGAM188 and VGAM189. FIG. 9 further provides a conceptual description of novel bioinformatically detected regulatory viral gene, referred to here as Viral Genomic Record 402(VGR402) viral gene, which encodes an ‘operon-like’ cluster of novel viral micro RNA-like genes, each of which in turn modulates expression of at least one host target gene, the function and utility of which at least one host target gene is known in the art.

VGR402 gene, herein designated VGR GENE, is a novel bioinformatically detected regulatory, non protein coding, RNA viral gene. The method by which VGR402 gene was detected is described hereinabove with reference to FIGS. 1-9.

VGR402 gene encodes VGR402 precursor RNA, herein designated VGR PRECURSOR RNA, an RNA molecule, typically several hundred nucleotides long.

VGR402 precursor RNA folds spatially, forming VGR402 folded precursor RNA, herein designated VGR FOLDED PRECURSOR RNA. It is appreciated that VGR402 folded precursor RNA comprises a plurality of what is known in the art as ‘hairpin’ structures. These ‘hairpin’ structures are due to the fact that the nucleotide sequence of VGR402 precursor RNA comprises a plurality of segments, the first half of each such segment having a nucleotide sequence which is at least a partial inversed-reversed sequence of the second half thereof, as is well known in the art.

VGR402 folded precursor RNA is naturally processed by cellular enzymatic activity into at least 2 separate VGAM precursor RNAs, VGAM193 precursor RNA and VGAM194 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM193 RNA and VGAM194 RNA, herein schematically represented by VGAM1 RNA through VGAM3 RNA, each of which VGAM RNAs corresponding to VGAM RNA of FIG. 1.

VGAM193 RNA, herein schematically represented by VGAM1 RNA through VGAM3 RNA, binds complementarily to a host target binding site located in an untranslated region of VGAM193 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM193 host target RNA into VGAM193 host target protein, herein schematically represented by VGAM1 HOST TARGET PROTEIN through VGAM3 HOST TARGET PROTEIN, all of FIG. 1.

VGAM194 RNA, herein schematically represented by VGAM1 RNA through VGAM3 RNA, binds complementarily to a host target binding site located in an untranslated region of VGAM194 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM194 host target RNA into VGAM194 host target protein, herein schematically represented by VGAM1 HOST TARGET PROTEIN through VGAM3 HOST TARGET PROTEIN, all of FIG. 1.

It is appreciated that a function of VGR402 gene, herein designated VGR GENE, is inhibition of expression of host target genes, as part of a novel viral mechanism of attacking a host. Accordingly, utilities of VGR402 gene include diagnosis, prevention and treatment of viral infection by Vaccinia Virus. Specific functions, and accordingly utilities, of VGR402 gene correlate with, and may be deduced from, the identity of the host target genes, which are inhibited by VGAM RNAs comprised in the ‘operon-like’ cluster of VGR402 gene: VGAM193 host target protein and VGAM194 host target protein, herein schematically represented by VGAM1 HOST TARGET PROTEIN through VGAM3 HOST TARGET PROTEIN. The function of these host target genes is elaborated hereinabove with reference to VGAM193 and VGAM194. FIG. 9 further provides a conceptual description of novel bioinformatically detected regulatory viral gene, referred to here as Viral Genomic Record 403(VGR403) viral gene, which encodes an ‘operon-like’ cluster of novel viral micro RNA-like genes, each of which in turn modulates expression of at least one host target gene, the function and utility of which at least one host target gene is known in the art.

VGR403 gene, herein designated VGR GENE, is a novel bioinformatically detected regulatory, non protein coding, RNA viral gene. The method by which VGR403 gene was detected is described hereinabove with reference to FIGS. 1-9.

VGR403 gene encodes VGR403 precursor RNA, herein designated VGR PRECURSOR RNA, an RNA molecule, typically several hundred nucleotides long.

VGR403 precursor RNA folds spatially, forming VGR403 folded precursor RNA, herein designated VGR FOLDED PRECURSOR RNA. It is appreciated that VGR403 folded precursor RNA comprises a plurality of what is known in the art as ‘hairpin’ structures. These ‘hairpin’ structures are due to the fact that the nucleotide sequence of VGR403 precursor RNA comprises a plurality of segments, the first half of each such segment having a nucleotide sequence which is at least a partial inversed-reversed sequence of the second half thereof, as is well known in the art.

VGR403 folded precursor RNA is naturally processed by cellular enzymatic activity into at least 3 separate VGAM precursor RNAs, VGAM195 precursor RNA, VGAM196 precursor RNA and VGAM197 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM195 RNA, VGAM196 RNA and VGAM197 RNA, herein schematically represented by VGAM1 RNA through VGAM3 RNA, each of which VGAM RNAs corresponding to VGAM RNA of FIG. 1.

VGAM195 RNA, herein schematically represented by VGAM1 RNA through VGAM3 RNA, binds complementarily to a host target binding site located in an untranslated region of VGAM195 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM195 host target RNA into VGAM195 host target protein, herein schematically represented by VGAM1 HOST TARGET PROTEIN through VGAM3 HOST TARGET PROTEIN, all of FIG. 1.

VGAM196 RNA, herein schematically represented by VGAM1 RNA through VGAM3 RNA, binds complementarily to a host target binding site located in an untranslated region of VGAM196 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM196 host target RNA into VGAM196 host target protein, herein schematically represented by VGAM1 HOST TARGET PROTEIN through VGAM3 HOST TARGET PROTEIN, all of FIG. 1.

VGAM197 RNA, herein schematically represented by VGAM1 RNA through VGAM3 RNA, binds complementarily to a host target binding site located in an untranslated region of VGAM197 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM197 host target RNA into VGAM197 host target protein, herein schematically represented by VGAM1 HOST TARGET PROTEIN through VGAM3 HOST TARGET PROTEIN, all of FIG. 1.

It is appreciated that a function of VGR403 gene, herein designated VGR GENE, is inhibition of expression of host target genes, as part of a novel viral mechanism of attacking a host. Accordingly, utilities of VGR403 gene include diagnosis, prevention and treatment of viral infection by Vaccinia Virus. Specific functions, and accordingly utilities, of VGR403 gene correlate with, and may be deduced from, the identity of the host target genes, which are inhibited by VGAM RNAs comprised in the ‘operon-like’ cluster of VGR403 gene: VGAM195 host target protein, VGAM196 host target protein and VGAM197 host target protein, herein schematically represented by VGAM1 HOST TARGET PROTEIN through VGAM3 HOST TARGET PROTEIN. The function of these host target genes is elaborated hereinabove with reference to VGAM195, VGAM196 and VGAM197. FIG. 9 further provides a conceptual description of novel bioinformatically detected regulatory viral gene, referred to here as Viral Genomic Record 404(VGR404) viral gene, which encodes an ‘operon-like’ cluster of novel viral micro RNA-like genes, each of which in turn modulates expression of at least one host target gene, the function and utility of which at least one host target gene is known in the art.

VGR404 gene, herein designated VGR GENE, is a novel bioinformatically detected regulatory, non protein coding, RNA viral gene. The method by which VGR404 gene was detected is described hereinabove with reference to FIGS. 1-9.

VGR404 gene encodes VGR404 precursor RNA, herein designated VGR PRECURSOR RNA, an RNA molecule, typically several hundred nucleotides long.

VGR404 precursor RNA folds spatially, forming VGR404 folded precursor RNA, herein designated VGR FOLDED PRECURSOR RNA. It is appreciated that VGR404 folded precursor RNA comprises a plurality of what is known in the art as ‘hairpin’ structures. These ‘hairpin’ structures are due to the fact that the nucleotide sequence of VGR404 precursor RNA comprises a plurality of segments, the first half of each such segment having a nucleotide sequence which is at least a partial inversed-reversed sequence of the second half thereof, as is well known in the art.

VGR404 folded precursor RNA is naturally processed by cellular enzymatic activity into at least 3 separate VGAM precursor RNAs, VGAM198 precursor RNA, VGAM199 precursor RNA and VGAM200 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM198 RNA, VGAM199 RNA and VGAM200 RNA, herein schematically represented by VGAM1 RNA through VGAM3 RNA, each of which VGAM RNAs corresponding to VGAM RNA of FIG. 1.

VGAM198 RNA, herein schematically represented by VGAM1 RNA through VGAM3 RNA, binds complementarily to a host target binding site located in an untranslated region of VGAM198 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM198 host target RNA into VGAM198 host target protein, herein schematically represented by VGAM1 HOST TARGET PROTEIN through VGAM3 HOST TARGET PROTEIN, all of FIG. 1.

VGAM199 RNA, herein schematically represented by VGAM1 RNA through VGAM3 RNA, binds complementarily to a host target binding site located in an untranslated region of VGAM199 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM199 host target RNA into VGAM199 host target protein, herein schematically represented by VGAM1 HOST TARGET PROTEIN through VGAM3 HOST TARGET PROTEIN, all of FIG. 1.

VGAM200 RNA, herein schematically represented by VGAM1 RNA through VGAM3 RNA, binds complementarily to a host target binding site located in an untranslated region of VGAM200 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM200 host target RNA into VGAM200 host target protein, herein schematically represented by VGAM1 HOST TARGET PROTEIN through VGAM3 HOST TARGET PROTEIN, all of FIG. 1.

It is appreciated that a function of VGR404 gene, herein designated VGR GENE, is inhibition of expression of host target genes, as part of a novel viral mechanism of attacking a host. Accordingly, utilities of VGR404 gene include diagnosis, prevention and treatment of viral infection by Vaccinia Virus. Specific functions, and accordingly utilities, of VGR404 gene correlate with, and may be deduced from, the identity of the host target genes, which are inhibited by VGAM RNAs comprised in the ‘operon-like’ cluster of VGR404 gene: VGAM198 host target protein, VGAM199 host target protein and VGAM200 host target protein, herein schematically represented by VGAM1 HOST TARGET PROTEIN through VGAM3 HOST TARGET PROTEIN. The function of these host target genes is elaborated hereinabove with reference to VGAM198, VGAM199 and VGAM200. FIG. 9 further provides a conceptual description of novel bioinformatically detected regulatory viral gene, referred to here as Viral Genomic Record 405(VGR405) viral gene, which encodes an ‘operon-like’ cluster of novel viral micro RNA-like genes, each of which in turn modulates expression of at least one host target gene, the function and utility of which at least one host target gene is known in the art.

VGR405 gene, herein designated VGR GENE, is a novel bioinformatically detected regulatory, non protein coding, RNA viral gene. The method by which VGR405 gene was detected is described hereinabove with reference to FIGS. 1-9.

VGR405 gene encodes VGR405 precursor RNA, herein designated VGR PRECURSOR RNA, an RNA molecule, typically several hundred nucleotides long.

VGR405 precursor RNA folds spatially, forming VGR405 folded precursor RNA, herein designated VGR FOLDED PRECURSOR RNA. It is appreciated that VGR405 folded precursor RNA comprises a plurality of what is known in the art as ‘hairpin’ structures. These ‘hairpin’ structures are due to the fact that the nucleotide sequence of VGR405 precursor RNA comprises a plurality of segments, the first half of each such segment having a nucleotide sequence which is at least a partial inversed-reversed sequence of the second half thereof, as is well known in the art.

VGR405 folded precursor RNA is naturally processed by cellular enzymatic activity into at least 2 separate VGAM precursor RNAs, VGAM201 precursor RNA and VGAM202 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM201 RNA and VGAM202 RNA, herein schematically represented by VGAM1 RNA through VGAM3 RNA, each of which VGAM RNAs corresponding to VGAM RNA of FIG. 1.

VGAM201 RNA, herein schematically represented by VGAM1 RNA through VGAM3 RNA, binds complementarily to a host target binding site located in an untranslated region of VGAM201 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM201 host target RNA into VGAM201 host target protein, herein schematically represented by VGAM1 HOST TARGET PROTEIN through VGAM3 HOST TARGET PROTEIN, all of FIG. 1.

VGAM202 RNA, herein schematically represented by VGAM1 RNA through VGAM3 RNA, binds complementarily to a host target binding site located in an untranslated region of VGAM202 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM202 host target RNA into VGAM202 host target protein, herein schematically represented by VGAM1 HOST TARGET PROTEIN through VGAM3 HOST TARGET PROTEIN, all of FIG. 1.

It is appreciated that a function of VGR405 gene, herein designated VGR GENE, is inhibition of expression of host target genes, as part of a novel viral mechanism of attacking a host. Accordingly, utilities of VGR405 gene include diagnosis, prevention and treatment of viral infection by Vaccinia Virus. Specific functions, and accordingly utilities, of VGR405 gene correlate with, and may be deduced from, the identity of the host target genes, which are inhibited by VGAM RNAs comprised in the ‘operon-like’ cluster of VGR405 gene: VGAM201 host target protein and VGAM202 host target protein, herein schematically represented by VGAM1 HOST TARGET PROTEIN through VGAM3 HOST TARGET PROTEIN. The function of these host target genes is elaborated hereinabove with reference to VGAM201 and VGAM202. FIG. 9 further provides a conceptual description of novel bioinformatically detected regulatory viral gene, referred to here as Viral Genomic Record 406(VGR406) viral gene, which encodes an ‘operon-like’ cluster of novel viral micro RNA-like genes, each of which in turn modulates expression of at least one host target gene, the function and utility of which at least one host target gene is known in the art.

VGR406 gene, herein designated VGR GENE, is a novel bioinformatically detected regulatory, non protein coding, RNA viral gene. The method by which VGR406 gene was detected is described hereinabove with reference to FIGS. 1-9.

VGR406 gene encodes VGR406 precursor RNA, herein designated VGR PRECURSOR RNA, an RNA molecule, typically several hundred nucleotides long.

VGR406 precursor RNA folds spatially, forming VGR406 folded precursor RNA, herein designated VGR FOLDED PRECURSOR RNA. It is appreciated that VGR406 folded precursor RNA comprises a plurality of what is known in the art as ‘hairpin’ structures. These ‘hairpin’ structures are due to the fact that the nucleotide sequence of VGR406 precursor RNA comprises a plurality of segments, the first half of each such segment having a nucleotide sequence which is at least a partial inversed-reversed sequence of the second half thereof, as is well known in the art.

VGR406 folded precursor RNA is naturally processed by cellular enzymatic activity into at least 3 separate VGAM precursor RNAs, VGAM203 precursor RNA, VGAM204 precursor RNA and VGAM205 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM203 RNA, VGAM204 RNA and VGAM205 RNA, herein schematically represented by VGAM1 RNA through VGAM3 RNA, each of which VGAM RNAs corresponding to VGAM RNA of FIG. 1.

VGAM203 RNA, herein schematically represented by VGAM1 RNA through VGAM3 RNA, binds complementarily to a host target binding site located in an untranslated region of VGAM203 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM203 host target RNA into VGAM203 host target protein, herein schematically represented by VGAM1 HOST TARGET PROTEIN through VGAM3 HOST TARGET PROTEIN, all of FIG. 1.

VGAM204 RNA, herein schematically represented by VGAM1 RNA through VGAM3 RNA, binds complementarily to a host target binding site located in an untranslated region of VGAM204 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM204 host target RNA into VGAM204 host target protein, herein schematically represented by VGAM1 HOST TARGET PROTEIN through VGAM3 HOST TARGET PROTEIN, all of FIG. 1.

VGAM205 RNA, herein schematically represented by VGAM1 RNA through VGAM3 RNA, binds complementarily to a host target binding site located in an untranslated region of VGAM205 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM205 host target RNA into VGAM205 host target protein, herein schematically represented by VGAM1 HOST TARGET PROTEIN through VGAM3 HOST TARGET PROTEIN, all of FIG. 1.

It is appreciated that a function of VGR406 gene, herein designated VGR GENE, is inhibition of expression of host target genes, as part of a novel viral mechanism of attacking a host. Accordingly, utilities of VGR406 gene include diagnosis, prevention and treatment of viral infection by Vaccinia Virus. Specific functions, and accordingly utilities, of VGR406 gene correlate with, and may be deduced from, the identity of the host target genes, which are inhibited by VGAM RNAs comprised in the ‘operon-like’ cluster of VGR406 gene: VGAM203 host target protein, VGAM204 host target protein and VGAM205 host target protein, herein schematically represented by VGAM1 HOST TARGET PROTEIN through VGAM3 HOST TARGET PROTEIN. The function of these host target genes is elaborated hereinabove with reference to VGAM203, VGAM204 and VGAM205. FIG. 9 further provides a conceptual description of novel bioinformatically detected regulatory viral gene, referred to here as Viral Genomic Record 407(VGR407) viral gene, which encodes an ‘operon-like’ cluster of novel viral micro RNA-like genes, each of which in turn modulates expression of at least one host target gene, the function and utility of which at least one host target gene is known in the art.

VGR407 gene, herein designated VGR GENE, is a novel bioinformatically detected regulatory, non protein coding, RNA viral gene. The method by which VGR407 gene was detected is described hereinabove with reference to FIGS. 1-9.

VGR407 gene encodes VGR407 precursor RNA, herein designated VGR PRECURSOR RNA, an RNA molecule, typically several hundred nucleotides long.

VGR407 precursor RNA folds spatially, forming VGR407 folded precursor RNA, herein designated VGR FOLDED PRECURSOR RNA. It is appreciated that VGR407 folded precursor RNA comprises a plurality of what is known in the art as ‘hairpin’ structures. These ‘hairpin’ structures are due to the fact that the nucleotide sequence of VGR407 precursor RNA comprises a plurality of segments, the first half of each such segment having a nucleotide sequence which is at least a partial inversed-reversed sequence of the second half thereof, as is well known in the art.

VGR407 folded precursor RNA is naturally processed by cellular enzymatic activity into at least 4 separate VGAM precursor RNAs, VGAM206 precursor RNA, VGAM207 precursor RNA, VGAM208 precursor RNA and VGAM209 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM206 RNA, VGAM207 RNA, VGAM208 RNA and VGAM209 RNA, herein schematically represented by VGAM1 RNA through VGAM3 RNA, each of which VGAM RNAs corresponding to VGAM RNA of FIG. 1.

VGAM206 RNA, herein schematically represented by VGAM1 RNA through VGAM3 RNA, binds complementarily to a host target binding site located in an untranslated region of VGAM206 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM206 host target RNA into VGAM206 host target protein, herein schematically represented by VGAM1 HOST TARGET PROTEIN through VGAM3 HOST TARGET PROTEIN, all of FIG. 1.

VGAM207 RNA, herein schematically represented by VGAM1 RNA through VGAM3 RNA, binds complementarily to a host target binding site located in an untranslated region of VGAM207 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM207 host target RNA into VGAM207 host target protein, herein schematically represented by VGAM1 HOST TARGET PROTEIN through VGAM3 HOST TARGET PROTEIN, all of FIG. 1.

VGAM208 RNA, herein schematically represented by VGAM1 RNA through VGAM3 RNA, binds complementarily to a host target binding site located in an untranslated region of VGAM208 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM208 host target RNA into VGAM208 host target protein, herein schematically represented by VGAM1 HOST TARGET PROTEIN through VGAM3 HOST TARGET PROTEIN, all of FIG. 1.

VGAM209 RNA, herein schematically represented by VGAM1 RNA through VGAM3 RNA, binds complementarily to a host target binding site located in an untranslated region of VGAM209 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM209 host target RNA into VGAM209 host target protein, herein schematically represented by VGAM1 HOST TARGET PROTEIN through VGAM3 HOST TARGET PROTEIN, all of FIG. 1.

It is appreciated that a function of VGR407 gene, herein designated VGR GENE, is inhibition of expression of host target genes, as part of a novel viral mechanism of attacking a host. Accordingly, utilities of VGR407 gene include diagnosis, prevention and treatment of viral infection by Vaccinia Virus. Specific functions, and accordingly utilities, of VGR407 gene correlate with, and may be deduced from, the identity of the host target genes, which are inhibited by VGAM RNAs comprised in the ‘operon-like’ cluster of VGR407 gene: VGAM206 host target protein, VGAM207 host target protein, VGAM208 host target protein and VGAM209 host target protein, herein schematically represented by VGAM1 HOST TARGET PROTEIN through VGAM3 HOST TARGET PROTEIN. The function of these host target genes is elaborated hereinabove with reference to VGAM206, VGAM207, VGAM208 and VGAM209. FIG. 9 further provides a conceptual description of novel bioinformatically detected regulatory viral gene, referred to here as Viral Genomic Record 408(VGR408) viral gene, which encodes an ‘operon-like’ cluster of novel viral micro RNA-like genes, each of which in turn modulates expression of at least one host target gene, the function and utility of which at least one host target gene is known in the art.

VGR408 gene, herein designated VGR GENE, is a novel bioinformatically detected regulatory, non protein coding, RNA viral gene. The method by which VGR408 gene was detected is described hereinabove with reference to FIGS. 1-9.

VGR408 gene encodes VGR408 precursor RNA, herein designated VGR PRECURSOR RNA, an RNA molecule, typically several hundred nucleotides long.

VGR408 precursor RNA folds spatially, forming VGR408 folded precursor RNA, herein designated VGR FOLDED PRECURSOR RNA. It is appreciated that VGR408 folded precursor RNA comprises a plurality of what is known in the art as ‘hairpin’ structures. These ‘hairpin’ structures are due to the fact that the nucleotide sequence of VGR408 precursor RNA comprises a plurality of segments, the first half of each such segment having a nucleotide sequence which is at least a partial inversed-reversed sequence of the second half thereof, as is well known in the art.

VGR408 folded precursor RNA is naturally processed by cellular enzymatic activity into at least 4 separate VGAM precursor RNAs, VGAM210 precursor RNA, VGAM211 precursor RNA, VGAM212 precursor RNA and VGAM213 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM210 RNA, VGAM211 RNA, VGAM212 RNA and VGAM213 RNA, herein schematically represented by VGAM1 RNA through VGAM3 RNA, each of which VGAM RNAs corresponding to VGAM RNA of FIG. 1.

VGAM210 RNA, herein schematically represented by VGAM1 RNA through VGAM3 RNA, binds complementarily to a host target binding site located in an untranslated region of VGAM210 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM210 host target RNA into VGAM210 host target protein, herein schematically represented by VGAM1 HOST TARGET PROTEIN through VGAM3 HOST TARGET PROTEIN, all of FIG. 1.

VGAM211 RNA, herein schematically represented by VGAM1 RNA through VGAM3 RNA, binds complementarily to a host target binding site located in an untranslated region of VGAM211 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM211 host target RNA into VGAM211 host target protein, herein schematically represented by VGAM1 HOST TARGET PROTEIN through VGAM3 HOST TARGET PROTEIN, all of FIG. 1.

VGAM212 RNA, herein schematically represented by VGAM1 RNA through VGAM3 RNA, binds complementarily to a host target binding site located in an untranslated region of VGAM212 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM212 host target RNA into VGAM212 host target protein, herein schematically represented by VGAM1 HOST TARGET PROTEIN through VGAM3 HOST TARGET PROTEIN, all of FIG. 1.

VGAM213 RNA, herein schematically represented by VGAM1 RNA through VGAM3 RNA, binds complementarily to a host target binding site located in an untranslated region of VGAM213 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM213 host target RNA into VGAM213 host target protein, herein schematically represented by VGAM1 HOST TARGET PROTEIN through VGAM3 HOST TARGET PROTEIN, all of FIG. 1.

It is appreciated that a function of VGR408 gene, herein designated VGR GENE, is inhibition of expression of host target genes, as part of a novel viral mechanism of attacking a host. Accordingly, utilities of VGR408 gene include diagnosis, prevention and treatment of viral infection by Vaccinia Virus. Specific functions, and accordingly utilities, of VGR408 gene correlate with, and may be deduced from, the identity of the host target genes, which are inhibited by VGAM RNAs comprised in the ‘operon-like’ cluster of VGR408 gene: VGAM210 host target protein, VGAM211 host target protein, VGAM212 host target protein and VGAM213 host target protein, herein schematically represented by VGAM1 HOST TARGET PROTEIN through VGAM3 HOST TARGET PROTEIN. The function of these host target genes is elaborated hereinabove with reference to VGAM210, VGAM211, VGAM212 and VGAM213. FIG. 9 further provides a conceptual description of novel bioinformatically detected regulatory viral gene, referred to here as Viral Genomic Record 409(VGR409) viral gene, which encodes an ‘operon-like’ cluster of novel viral micro RNA-like genes, each of which in turn modulates expression of at least one host target gene, the function and utility of which at least one host target gene is known in the art.

VGR409 gene, herein designated VGR GENE, is a novel bioinformatically detected regulatory, non protein coding, RNA viral gene. The method by which VGR409 gene was detected is described hereinabove with reference to FIGS. 1-9.

VGR409 gene encodes VGR409 precursor RNA, herein designated VGR PRECURSOR RNA, an RNA molecule, typically several hundred nucleotides long.

VGR409 precursor RNA folds spatially, forming VGR409 folded precursor RNA, herein designated VGR FOLDED PRECURSOR RNA. It is appreciated that VGR409 folded precursor RNA comprises a plurality of what is known in the art as ‘hairpin’ structures. These ‘hairpin’ structures are due to the fact that the nucleotide sequence of VGR409 precursor RNA comprises a plurality of segments, the first half of each such segment having a nucleotide sequence which is at least a partial inversed-reversed sequence of the second half thereof, as is well known in the art.

VGR409 folded precursor RNA is naturally processed by cellular enzymatic activity into at least 4 separate VGAM precursor RNAs, VGAM214 precursor RNA, VGAM215 precursor RNA, VGAM216 precursor RNA and VGAM217 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM214 RNA, VGAM215 RNA, VGAM216 RNA and VGAM217 RNA, herein schematically represented by VGAM1 RNA through VGAM3 RNA, each of which VGAM RNAs corresponding to VGAM RNA of FIG. 1.

VGAM214 RNA, herein schematically represented by VGAM1 RNA through VGAM3 RNA, binds complementarily to a host target binding site located in an untranslated region of VGAM214 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM214 host target RNA into VGAM214 host target protein, herein schematically represented by VGAM1 HOST TARGET PROTEIN through VGAM3 HOST TARGET PROTEIN, all of FIG. 1.

VGAM215 RNA, herein schematically represented by VGAM1 RNA through VGAM3 RNA, binds complementarily to a host target binding site located in an untranslated region of VGAM215 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM215 host target RNA into VGAM215 host target protein, herein schematically represented by VGAM1 HOST TARGET PROTEIN through VGAM3 HOST TARGET PROTEIN, all of FIG. 1.

VGAM216 RNA, herein schematically represented by VGAM1 RNA through VGAM3 RNA, binds complementarily to a host target binding site located in an untranslated region of VGAM216 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM216 host target RNA into VGAM216 host target protein, herein schematically represented by VGAM1 HOST TARGET PROTEIN through VGAM3 HOST TARGET PROTEIN, all of FIG. 1.

VGAM217 RNA, herein schematically represented by VGAM1 RNA through VGAM3 RNA, binds complementarily to a host target binding site located in an untranslated region of VGAM217 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM217 host target RNA into VGAM217 host target protein, herein schematically represented by VGAM1 HOST TARGET PROTEIN through VGAM3 HOST TARGET PROTEIN, all of FIG. 1.

It is appreciated that a function of VGR409 gene, herein designated VGR GENE, is inhibition of expression of host target genes, as part of a novel viral mechanism of attacking a host. Accordingly, utilities of VGR409 gene include diagnosis, prevention and treatment of viral infection by Vaccinia Virus. Specific functions, and accordingly utilities, of VGR409 gene correlate with, and may be deduced from, the identity of the host target genes, which are inhibited by VGAM RNAs comprised in the ‘operon-like’ cluster of VGR409 gene: VGAM214 host target protein, VGAM215 host target protein, VGAM216 host target protein and VGAM217 host target protein, herein schematically represented by VGAM1 HOST TARGET PROTEIN through VGAM3 HOST TARGET PROTEIN. The function of these host target genes is elaborated hereinabove with reference to VGAM214, VGAM215, VGAM216 and VGAM217. FIG. 9 further provides a conceptual description of novel bioinformatically detected regulatory viral gene, referred to here as Viral Genomic Record 410(VGR410) viral gene, which encodes an ‘operon-like’ cluster of novel viral micro RNA-like genes, each of which in turn modulates expression of at least one host target gene, the function and utility of which at least one host target gene is known in the art.

VGR410 gene, herein designated VGR GENE, is a novel bioinformatically detected regulatory, non protein coding, RNA viral gene. The method by which VGR410 gene was detected is described hereinabove with reference to FIGS. 1-9.

VGR410 gene encodes VGR410 precursor RNA, herein designated VGR PRECURSOR RNA, an RNA molecule, typically several hundred nucleotides long.

VGR410 precursor RNA folds spatially, forming VGR410 folded precursor RNA, herein designated VGR FOLDED PRECURSOR RNA. It is appreciated that VGR410 folded precursor RNA comprises a plurality of what is known in the art as ‘hairpin’ structures. These ‘hairpin’ structures are due to the fact that the nucleotide sequence of VGR410 precursor RNA comprises a plurality of segments, the first half of each such segment having a nucleotide sequence which is at least a partial inversed-reversed sequence of the second half thereof, as is well known in the art.

VGR410 folded precursor RNA is naturally processed by cellular enzymatic activity into at least 3 separate VGAM precursor RNAs, VGAM219 precursor RNA, VGAM220 precursor RNA and VGAM221 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM219 RNA, VGAM220 RNA and VGAM221 RNA, herein schematically represented by VGAM1 RNA through VGAM3 RNA, each of which VGAM RNAs corresponding to VGAM RNA of FIG. 1.

VGAM219 RNA, herein schematically represented by VGAM1 RNA through VGAM3 RNA, binds complementarily to a host target binding site located in an untranslated region of VGAM219 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM219 host target RNA into VGAM219 host target protein, herein schematically represented by VGAM1 HOST TARGET PROTEIN through VGAM3 HOST TARGET PROTEIN, all of FIG. 1.

VGAM220 RNA, herein schematically represented by VGAM1 RNA through VGAM3 RNA, binds complementarily to a host target binding site located in an untranslated region of VGAM220 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM220 host target RNA into VGAM220 host target protein, herein schematically represented by VGAM1 HOST TARGET PROTEIN through VGAM3 HOST TARGET PROTEIN, all of FIG. 1.

VGAM221 RNA, herein schematically represented by VGAM1 RNA through VGAM3 RNA, binds complementarily to a host target binding site located in an untranslated region of VGAM221 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM221 host target RNA into VGAM221 host target protein, herein schematically represented by VGAM1 HOST TARGET PROTEIN through VGAM3 HOST TARGET PROTEIN, all of FIG. 1.

It is appreciated that a function of VGR410 gene, herein designated VGR GENE, is inhibition of expression of host target genes, as part of a novel viral mechanism of attacking a host. Accordingly, utilities of VGR410 gene include diagnosis, prevention and treatment of viral infection by Vaccinia Virus. Specific functions, and accordingly utilities, of VGR410 gene correlate with, and may be deduced from, the identity of the host target genes, which are inhibited by VGAM RNAs comprised in the ‘operon-like’ cluster of VGR410 gene: VGAM219 host target protein, VGAM220 host target protein and VGAM221 host target protein, herein schematically represented by VGAM1 HOST TARGET PROTEIN through VGAM3 HOST TARGET PROTEIN. The function of these host target genes is elaborated hereinabove with reference to VGAM219, VGAM220 and VGAM221. FIG. 9 further provides a conceptual description of novel bioinformatically detected regulatory viral gene, referred to here as Viral Genomic Record 411(VGR411) viral gene, which encodes an ‘operon-like’ cluster of novel viral micro RNA-like genes, each of which in turn modulates expression of at least one host target gene, the function and utility of which at least one host target gene is known in the art.

VGR411 gene, herein designated VGR GENE, is a novel bioinformatically detected regulatory, non protein coding, RNA viral gene. The method by which VGR411 gene was detected is described hereinabove with reference to FIGS. 1-9.

VGR411 gene encodes VGR411 precursor RNA, herein designated VGR PRECURSOR RNA, an RNA molecule, typically several hundred nucleotides long.

VGR411 precursor RNA folds spatially, forming VGR411 folded precursor RNA, herein designated VGR FOLDED PRECURSOR RNA. It is appreciated that VGR411 folded precursor RNA comprises a plurality of what is known in the art as ‘hairpin’ structures. These ‘hairpin’ structures are due to the fact that the nucleotide sequence of VGR411 precursor RNA comprises a plurality of segments, the first half of each such segment having a nucleotide sequence which is at least a partial inversed-reversed sequence of the second half thereof, as is well known in the art.

VGR411 folded precursor RNA is naturally processed by cellular enzymatic activity into at least 2 separate VGAM precursor RNAs, VGAM222 precursor RNA and VGAM223 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM222 RNA and VGAM223 RNA, herein schematically represented by VGAM1 RNA through VGAM3 RNA, each of which VGAM RNAs corresponding to VGAM RNA of FIG. 1.

VGAM222 RNA, herein schematically represented by VGAM1 RNA through VGAM3 RNA, binds complementarily to a host target binding site located in an untranslated region of VGAM222 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM222 host target RNA into VGAM222 host target protein, herein schematically represented by VGAM1 HOST TARGET PROTEIN through VGAM3 HOST TARGET PROTEIN, all of FIG. 1.

VGAM223 RNA, herein schematically represented by VGAM1 RNA through VGAM3 RNA, binds complementarily to a host target binding site located in an untranslated region of VGAM223 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM223 host target RNA into VGAM223 host target protein, herein schematically represented by VGAM1 HOST TARGET PROTEIN through VGAM3 HOST TARGET PROTEIN, all of FIG. 1.

It is appreciated that a function of VGR411 gene, herein designated VGR GENE, is inhibition of expression of host target genes, as part of a novel viral mechanism of attacking a host. Accordingly, utilities of VGR411 gene include diagnosis, prevention and treatment of viral infection by Vaccinia Virus. Specific functions, and accordingly utilities, of VGR411 gene correlate with, and may be deduced from, the identity of the host target genes, which are inhibited by VGAM RNAs comprised in the ‘operon-like’ cluster of VGR411 gene: VGAM222 host target protein and VGAM223 host target protein, herein schematically represented by VGAM1 HOST TARGET PROTEIN through VGAM3 HOST TARGET PROTEIN. The function of these host target genes is elaborated hereinabove with reference to VGAM222 and VGAM223. FIG. 9 further provides a conceptual description of novel bioinformatically detected regulatory viral gene, referred to here as Viral Genomic Record 412(VGR412) viral gene, which encodes an ‘operon-like’ cluster of novel viral micro RNA-like genes, each of which in turn modulates expression of at least one host target gene, the function and utility of which at least one host target gene is known in the art.

VGR412 gene, herein designated VGR GENE, is a novel bioinformatically detected regulatory, non protein coding, RNA viral gene. The method by which VGR412 gene was detected is described hereinabove with reference to FIGS. 1-9.

VGR412 gene encodes VGR412 precursor RNA, herein designated VGR PRECURSOR RNA, an RNA molecule, typically several hundred nucleotides long.

VGR412 precursor RNA folds spatially, forming VGR412 folded precursor RNA, herein designated VGR FOLDED PRECURSOR RNA. It is appreciated that VGR412 folded precursor RNA comprises a plurality of what is known in the art as ‘hairpin’ structures. These ‘hairpin’ structures are due to the fact that the nucleotide sequence of VGR412 precursor RNA comprises a plurality of segments, the first half of each such segment having a nucleotide sequence which is at least a partial inversed-reversed sequence of the second half thereof, as is well known in the art.

VGR412 folded precursor RNA is naturally processed by cellular enzymatic activity into at least 4 separate VGAM precursor RNAs, VGAM224 precursor RNA, VGAM225 precursor RNA, VGAM226 precursor RNA and VGAM227 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM224 RNA, VGAM225 RNA, VGAM226 RNA and VGAM227 RNA, herein schematically represented by VGAM1 RNA through VGAM3 RNA, each of which VGAM RNAs corresponding to VGAM RNA of FIG. 1.

VGAM224 RNA, herein schematically represented by VGAM1 RNA through VGAM3 RNA, binds complementarily to a host target binding site located in an untranslated region of VGAM224 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM224 host target RNA into VGAM224 host target protein, herein schematically represented by VGAM1 HOST TARGET PROTEIN through VGAM3 HOST TARGET PROTEIN, all of FIG. 1.

VGAM225 RNA, herein schematically represented by VGAM1 RNA through VGAM3 RNA, binds complementarily to a host target binding site located in an untranslated region of VGAM225 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM225 host target RNA into VGAM225 host target protein, herein schematically represented by VGAM1 HOST TARGET PROTEIN through VGAM3 HOST TARGET PROTEIN, all of FIG. 1.

VGAM226 RNA, herein schematically represented by VGAM1 RNA through VGAM3 RNA, binds complementarily to a host target binding site located in an untranslated region of VGAM226 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM226 host target RNA into VGAM226 host target protein, herein schematically represented by VGAM1 HOST TARGET PROTEIN through VGAM3 HOST TARGET PROTEIN, all of FIG. 1.

VGAM227 RNA, herein schematically represented by VGAM1 RNA through VGAM3 RNA, binds complementarily to a host target binding site located in an untranslated region of VGAM227 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM227 host target RNA into VGAM227 host target protein, herein schematically represented by VGAM1 HOST TARGET PROTEIN through VGAM3 HOST TARGET PROTEIN, all of FIG. 1.

It is appreciated that a function of VGR412 gene, herein designated VGR GENE, is inhibition of expression of host target genes, as part of a novel viral mechanism of attacking a host. Accordingly, utilities of VGR412 gene include diagnosis, prevention and treatment of viral infection by Vaccinia Virus. Specific functions, and accordingly utilities, of VGR412 gene correlate with, and may be deduced from, the identity of the host target genes, which are inhibited by VGAM RNAs comprised in the ‘operon-like’ cluster of VGR412 gene: VGAM224 host target protein, VGAM225 host target protein, VGAM226 host target protein and VGAM227 host target protein, herein schematically represented by VGAM1 HOST TARGET PROTEIN through VGAM3 HOST TARGET PROTEIN. The function of these host target genes is elaborated hereinabove with reference to VGAM224, VGAM225, VGAM226 and VGAM227. FIG. 9 further provides a conceptual description of novel bioinformatically detected regulatory viral gene, referred to here as Viral Genomic Record 413(VGR413) viral gene, which encodes an ‘operon-like’ cluster of novel viral micro RNA-like genes, each of which in turn modulates expression of at least one host target gene, the function and utility of which at least one host target gene is known in the art.

VGR413 gene, herein designated VGR GENE, is a novel bioinformatically detected regulatory, non protein coding, RNA viral gene. The method by which VGR413 gene was detected is described hereinabove with reference to FIGS. 1-9.

VGR413 gene encodes VGR413 precursor RNA, herein designated VGR PRECURSOR RNA, an RNA molecule, typically several hundred nucleotides long.

VGR413 precursor RNA folds spatially, forming VGR413 folded precursor RNA, herein designated VGR FOLDED PRECURSOR RNA. It is appreciated that VGR413 folded precursor RNA comprises a plurality of what is known in the art as ‘hairpin’ structures. These ‘hairpin’ structures are due to the fact that the nucleotide sequence of VGR413 precursor RNA comprises a plurality of segments, the first half of each such segment having a nucleotide sequence which is at least a partial inversed-reversed sequence of the second half thereof, as is well known in the art.

VGR413 folded precursor RNA is naturally processed by cellular enzymatic activity into at least 4 separate VGAM precursor RNAs, VGAM230 precursor RNA, VGAM231 precursor RNA, VGAM232 precursor RNA and VGAM233 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM230 RNA, VGAM231 RNA, VGAM232 RNA and VGAM233 RNA, herein schematically represented by VGAM1 RNA through VGAM3 RNA, each of which VGAM RNAs corresponding to VGAM RNA of FIG. 1.

VGAM230 RNA, herein schematically represented by VGAM1 RNA through VGAM3 RNA, binds complementarily to a host target binding site located in an untranslated region of VGAM230 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM230 host target RNA into VGAM230 host target protein, herein schematically represented by VGAM1 HOST TARGET PROTEIN through VGAM3 HOST TARGET PROTEIN, all of FIG. 1.

VGAM231 RNA, herein schematically represented by VGAM1 RNA through VGAM3 RNA, binds complementarily to a host target binding site located in an untranslated region of VGAM231 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM231 host target RNA into VGAM231 host target protein, herein schematically represented by VGAM1 HOST TARGET PROTEIN through VGAM3 HOST TARGET PROTEIN, all of FIG. 1.

VGAM232 RNA, herein schematically represented by VGAM1 RNA through VGAM3 RNA, binds complementarily to a host target binding site located in an untranslated region of VGAM232 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM232 host target RNA into VGAM232 host target protein, herein schematically represented by VGAM1 HOST TARGET PROTEIN through VGAM3 HOST TARGET PROTEIN, all of FIG. 1.

VGAM233 RNA, herein schematically represented by VGAM1 RNA through VGAM3 RNA, binds complementarily to a host target binding site located in an untranslated region of VGAM233 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM233 host target RNA into VGAM233 host target protein, herein schematically represented by VGAM1 HOST TARGET PROTEIN through VGAM3 HOST TARGET PROTEIN, all of FIG. 1.

It is appreciated that a function of VGR413 gene, herein designated VGR GENE, is inhibition of expression of host target genes, as part of a novel viral mechanism of attacking a host. Accordingly, utilities of VGR413 gene include diagnosis, prevention and treatment of viral infection by Vaccinia Virus. Specific functions, and accordingly utilities, of VGR413 gene correlate with, and may be deduced from, the identity of the host target genes, which are inhibited by VGAM RNAs comprised in the ‘operon-like’ cluster of VGR413 gene: VGAM230 host target protein, VGAM231 host target protein, VGAM232 host target protein and VGAM233 host target protein, herein schematically represented by VGAM1 HOST TARGET PROTEIN through VGAM3 HOST TARGET PROTEIN. The function of these host target genes is elaborated hereinabove with reference to VGAM230, VGAM231, VGAM232 and VGAM233. FIG. 9 further provides a conceptual description of novel bioinformatically detected regulatory viral gene, referred to here as Viral Genomic Record 414(VGR414) viral gene, which encodes an ‘operon-like’ cluster of novel viral micro RNA-like genes, each of which in turn modulates expression of at least one host target gene, the function and utility of which at least one host target gene is known in the art.

VGR414 gene, herein designated VGR GENE, is a novel bioinformatically detected regulatory, non protein coding, RNA viral gene. The method by which VGR414 gene was detected is described hereinabove with reference to FIGS. 1-9.

VGR414 gene encodes VGR414 precursor RNA, herein designated VGR PRECURSOR RNA, an RNA molecule, typically several hundred nucleotides long.

VGR414 precursor RNA folds spatially, forming VGR414 folded precursor RNA, herein designated VGR FOLDED PRECURSOR RNA. It is appreciated that VGR414 folded precursor RNA comprises a plurality of what is known in the art as ‘hairpin’ structures. These ‘hairpin’ structures are due to the fact that the nucleotide sequence of VGR414 precursor RNA comprises a plurality of segments, the first half of each such segment having a nucleotide sequence which is at least a partial inversed-reversed sequence of the second half thereof, as is well known in the art.

VGR414 folded precursor RNA is naturally processed by cellular enzymatic activity into at least 2 separate VGAM precursor RNAs, VGAM234 precursor RNA and VGAM235 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM234 RNA and VGAM235 RNA, herein schematically represented by VGAM1 RNA through VGAM3 RNA, each of which VGAM RNAs corresponding to VGAM RNA of FIG. 1.

VGAM234 RNA, herein schematically represented by VGAM1 RNA through VGAM3 RNA, binds complementarily to a host target binding site located in an untranslated region of VGAM234 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM234 host target RNA into VGAM234 host target protein, herein schematically represented by VGAM1 HOST TARGET PROTEIN through VGAM3 HOST TARGET PROTEIN, all of FIG. 1.

VGAM235 RNA, herein schematically represented by VGAM1 RNA through VGAM3 RNA, binds complementarily to a host target binding site located in an untranslated region of VGAM235 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM235 host target RNA into VGAM235 host target protein, herein schematically represented by VGAM1 HOST TARGET PROTEIN through VGAM3 HOST TARGET PROTEIN, all of FIG. 1.

It is appreciated that a function of VGR414 gene, herein designated VGR GENE, is inhibition of expression of host target genes, as part of a novel viral mechanism of attacking a host. Accordingly, utilities of VGR414 gene include diagnosis, prevention and treatment of viral infection by Vaccinia Virus. Specific functions, and accordingly utilities, of VGR414 gene correlate with, and may be deduced from, the identity of the host target genes, which are inhibited by VGAM RNAs comprised in the ‘operon-like’ cluster of VGR414 gene: VGAM234 host target protein and VGAM235 host target protein, herein schematically represented by VGAM1 HOST TARGET PROTEIN through VGAM3 HOST TARGET PROTEIN. The function of these host target genes is elaborated hereinabove with reference to VGAM234 and VGAM235. FIG. 9 further provides a conceptual description of novel bioinformatically detected regulatory viral gene, referred to here as Viral Genomic Record 415(VGR415) viral gene, which encodes an ‘operon-like’ cluster of novel viral micro RNA-like genes, each of which in turn modulates expression of at least one host target gene, the function and utility of which at least one host target gene is known in the art.

VGR415 gene, herein designated VGR GENE, is a novel bioinformatically detected regulatory, non protein coding, RNA viral gene. The method by which VGR415 gene was detected is described hereinabove with reference to FIGS. 1-9.

VGR415 gene encodes VGR415 precursor RNA, herein designated VGR PRECURSOR RNA, an RNA molecule, typically several hundred nucleotides long.

VGR415 precursor RNA folds spatially, forming VGR415 folded precursor RNA, herein designated VGR FOLDED PRECURSOR RNA. It is appreciated that VGR415 folded precursor RNA comprises a plurality of what is known in the art as ‘hairpin’ structures. These ‘hairpin’ structures are due to the fact that the nucleotide sequence of VGR415 precursor RNA comprises a plurality of segments, the first half of each such segment having a nucleotide sequence which is at least a partial inversed-reversed sequence of the second half thereof, as is well known in the art.

VGR415 folded precursor RNA is naturally processed by cellular enzymatic activity into at least 2 separate VGAM precursor RNAs, VGAM237 precursor RNA and VGAM238 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM237 RNA and VGAM238 RNA, herein schematically represented by VGAM1 RNA through VGAM3 RNA, each of which VGAM RNAs corresponding to VGAM RNA of FIG. 1.

VGAM237 RNA, herein schematically represented by VGAM1 RNA through VGAM3 RNA, binds complementarily to a host target binding site located in an untranslated region of VGAM237 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM237 host target RNA into VGAM237 host target protein, herein schematically represented by VGAM1 HOST TARGET PROTEIN through VGAM3 HOST TARGET PROTEIN, all of FIG. 1.

VGAM238 RNA, herein schematically represented by VGAM1 RNA through VGAM3 RNA, binds complementarily to a host target binding site located in an untranslated region of VGAM238 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM238 host target RNA into VGAM238 host target protein, herein schematically represented by VGAM1 HOST TARGET PROTEIN through VGAM3 HOST TARGET PROTEIN, all of FIG. 1.

It is appreciated that a function of VGR415 gene, herein designated VGR GENE, is inhibition of expression of host target genes, as part of a novel viral mechanism of attacking a host. Accordingly, utilities of VGR415 gene include diagnosis, prevention and treatment of viral infection by Vaccinia Virus. Specific functions, and accordingly utilities, of VGR415 gene correlate with, and may be deduced from, the identity of the host target genes, which are inhibited by VGAM RNAs comprised in the ‘operon-like’ cluster of VGR415 gene: VGAM237 host target protein and VGAM238 host target protein, herein schematically represented by VGAM1 HOST TARGET PROTEIN through VGAM3 HOST TARGET PROTEIN. The function of these host target genes is elaborated hereinabove with reference to VGAM237 and VGAM238. FIG. 9 further provides a conceptual description of novel bioinformatically detected regulatory viral gene, referred to here as Viral Genomic Record 416(VGR416) viral gene, which encodes an ‘operon-like’ cluster of novel viral micro RNA-like genes, each of which in turn modulates expression of at least one host target gene, the function and utility of which at least one host target gene is known in the art.

VGR416 gene, herein designated VGR GENE, is a novel bioinformatically detected regulatory, non protein coding, RNA viral gene. The method by which VGR416 gene was detected is described hereinabove with reference to FIGS. 1-9.

VGR416 gene encodes VGR416 precursor RNA, herein designated VGR PRECURSOR RNA, an RNA molecule, typically several hundred nucleotides long.

VGR416 precursor RNA folds spatially, forming VGR416 folded precursor RNA, herein designated VGR FOLDED PRECURSOR RNA. It is appreciated that VGR416 folded precursor RNA comprises a plurality of what is known in the art as ‘hairpin’ structures. These ‘hairpin’ structures are due to the fact that the nucleotide sequence of VGR416 precursor RNA comprises a plurality of segments, the first half of each such segment having a nucleotide sequence which is at least a partial inversed-reversed sequence of the second half thereof, as is well known in the art.

VGR416 folded precursor RNA is naturally processed by cellular enzymatic activity into at least 2 separate VGAM precursor RNAs, VGAM240 precursor RNA and VGAM241 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM240 RNA and VGAM241 RNA, herein schematically represented by VGAM1 RNA through VGAM3 RNA, each of which VGAM RNAs corresponding to VGAM RNA of FIG. 1.

VGAM240 RNA, herein schematically represented by VGAM1 RNA through VGAM3 RNA, binds complementarily to a host target binding site located in an untranslated region of VGAM240 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM240 host target RNA into VGAM240 host target protein, herein schematically represented by VGAM1 HOST TARGET PROTEIN through VGAM3 HOST TARGET PROTEIN, all of FIG. 1.

VGAM241 RNA, herein schematically represented by VGAM1 RNA through VGAM3 RNA, binds complementarily to a host target binding site located in an untranslated region of VGAM241 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM241 host target RNA into VGAM241 host target protein, herein schematically represented by VGAM1 HOST TARGET PROTEIN through VGAM3 HOST TARGET PROTEIN, all of FIG. 1.

It is appreciated that a function of VGR416 gene, herein designated VGR GENE, is inhibition of expression of host target genes, as part of a novel viral mechanism of attacking a host. Accordingly, utilities of VGR416 gene include diagnosis, prevention and treatment of viral infection by Vaccinia Virus. Specific functions, and accordingly utilities, of VGR416 gene correlate with, and may be deduced from, the identity of the host target genes, which are inhibited by VGAM RNAs comprised in the ‘operon-like’ cluster of VGR416 gene: VGAM240 host target protein and VGAM241 host target protein, herein schematically represented by VGAM1 HOST TARGET PROTEIN through VGAM3 HOST TARGET PROTEIN. The function of these host target genes is elaborated hereinabove with reference to VGAM240 and VGAM241. FIG. 9 further provides a conceptual description of novel bioinformatically detected regulatory viral gene, referred to here as Viral Genomic Record 417(VGR417) viral gene, which encodes an ‘operon-like’ cluster of novel viral micro RNA-like genes, each of which in turn modulates expression of at least one host target gene, the function and utility of which at least one host target gene is known in the art.

VGR417 gene, herein designated VGR GENE, is a novel bioinformatically detected regulatory, non protein coding, RNA viral gene. The method by which VGR417 gene was detected is described hereinabove with reference to FIGS. 1-9.

VGR417 gene encodes VGR417 precursor RNA, herein designated VGR PRECURSOR RNA, an RNA molecule, typically several hundred nucleotides long.

VGR417 precursor RNA folds spatially, forming VGR417 folded precursor RNA, herein designated VGR FOLDED PRECURSOR RNA. It is appreciated that VGR417 folded precursor RNA comprises a plurality of what is known in the art as ‘hairpin’ structures. These ‘hairpin’ structures are due to the fact that the nucleotide sequence of VGR417 precursor RNA comprises a plurality of segments, the first half of each such segment having a nucleotide sequence which is at least a partial inversed-reversed sequence of the second half thereof, as is well known in the art.

VGR417 folded precursor RNA is naturally processed by cellular enzymatic activity into at least 4 separate VGAM precursor RNAs, VGAM243 precursor RNA, VGAM244 precursor RNA, VGAM245 precursor RNA and VGAM246 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM243 RNA, VGAM244 RNA, VGAM245 RNA and VGAM246 RNA, herein schematically represented by VGAM1 RNA through VGAM3 RNA, each of which VGAM RNAs corresponding to VGAM RNA of FIG. 1.

VGAM243 RNA, herein schematically represented by VGAM1 RNA through VGAM3 RNA, binds complementarily to a host target binding site located in an untranslated region of VGAM243 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM243 host target RNA into VGAM243 host target protein, herein schematically represented by VGAM1 HOST TARGET PROTEIN through VGAM3 HOST TARGET PROTEIN, all of FIG. 1.

VGAM244 RNA, herein schematically represented by VGAM1 RNA through VGAM3 RNA, binds complementarily to a host target binding site located in an untranslated region of VGAM244 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM244 host target RNA into VGAM244 host target protein, herein schematically represented by VGAM1 HOST TARGET PROTEIN through VGAM3 HOST TARGET PROTEIN, all of FIG. 1.

VGAM245 RNA, herein schematically represented by VGAM1 RNA through VGAM3 RNA, binds complementarily to a host target binding site located in an untranslated region of VGAM245 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM245 host target RNA into VGAM245 host target protein, herein schematically represented by VGAM1 HOST TARGET PROTEIN through VGAM3 HOST TARGET PROTEIN, all of FIG. 1.

VGAM246 RNA, herein schematically represented by VGAM1 RNA through VGAM3 RNA, binds complementarily to a host target binding site located in an untranslated region of VGAM246 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM246 host target RNA into VGAM246 host target protein, herein schematically represented by VGAM1 HOST TARGET PROTEIN through VGAM3 HOST TARGET PROTEIN, all of FIG. 1.

It is appreciated that a function of VGR417 gene, herein designated VGR GENE, is inhibition of expression of host target genes, as part of a novel viral mechanism of attacking a host. Accordingly, utilities of VGR417 gene include diagnosis, prevention and treatment of viral infection by Vaccinia Virus. Specific functions, and accordingly utilities, of VGR417 gene correlate with, and may be deduced from, the identity of the host target genes, which are inhibited by VGAM RNAs comprised in the ‘operon-like’ cluster of VGR417 gene: VGAM243 host target protein, VGAM244 host target protein, VGAM245 host target protein and VGAM246 host target protein, herein schematically represented by VGAM1 HOST TARGET PROTEIN through VGAM3 HOST TARGET PROTEIN. The function of these host target genes is elaborated hereinabove with reference to VGAM243, VGAM244, VGAM245 and VGAM246. FIG. 9 further provides a conceptual description of novel bioinformatically detected regulatory viral gene, referred to here as Viral Genomic Record 418(VGR418) viral gene, which encodes an ‘operon-like’ cluster of novel viral micro RNA-like genes, each of which in turn modulates expression of at least one host target gene, the function and utility of which at least one host target gene is known in the art.

VGR418 gene, herein designated VGR GENE, is a novel bioinformatically detected regulatory, non protein coding, RNA viral gene. The method by which VGR418 gene was detected is described hereinabove with reference to FIGS. 1-9.

VGR418 gene encodes VGR418 precursor RNA, herein designated VGR PRECURSOR RNA, an RNA molecule, typically several hundred nucleotides long.

VGR418 precursor RNA folds spatially, forming VGR418 folded precursor RNA, herein designated VGR FOLDED PRECURSOR RNA. It is appreciated that VGR418 folded precursor RNA comprises a plurality of what is known in the art as ‘hairpin’ structures. These ‘hairpin’ structures are due to the fact that the nucleotide sequence of VGR418 precursor RNA comprises a plurality of segments, the first half of each such segment having a nucleotide sequence which is at least a partial inversed-reversed sequence of the second half thereof, as is well known in the art.

VGR418 folded precursor RNA is naturally processed by cellular enzymatic activity into at least 4 separate VGAM precursor RNAs, VGAM247 precursor RNA, VGAM248 precursor RNA, VGAM249 precursor RNA and VGAM250 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM247 RNA, VGAM248 RNA, VGAM249 RNA and VGAM250 RNA, herein schematically represented by VGAM1 RNA through VGAM3 RNA, each of which VGAM RNAs corresponding to VGAM RNA of FIG. 1.

VGAM247 RNA, herein schematically represented by VGAM1 RNA through VGAM3 RNA, binds complementarily to a host target binding site located in an untranslated region of VGAM247 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM247 host target RNA into VGAM247 host target protein, herein schematically represented by VGAM1 HOST TARGET PROTEIN through VGAM3 HOST TARGET PROTEIN, all of FIG. 1.

VGAM248 RNA, herein schematically represented by VGAM1 RNA through VGAM3 RNA, binds complementarily to a host target binding site located in an untranslated region of VGAM248 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM248 host target RNA into VGAM248 host target protein, herein schematically represented by VGAM1 HOST TARGET PROTEIN through VGAM3 HOST TARGET PROTEIN, all of FIG. 1.

VGAM249 RNA, herein schematically represented by VGAM1 RNA through VGAM3 RNA, binds complementarily to a host target binding site located in an untranslated region of VGAM249 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM249 host target RNA into VGAM249 host target protein, herein schematically represented by VGAM1 HOST TARGET PROTEIN through VGAM3 HOST TARGET PROTEIN, all of FIG. 1.

VGAM250 RNA, herein schematically represented by VGAM1 RNA through VGAM3 RNA, binds complementarily to a host target binding site located in an untranslated region of VGAM250 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM250 host target RNA into VGAM250 host target protein, herein schematically represented by VGAM1 HOST TARGET PROTEIN through VGAM3 HOST TARGET PROTEIN, all of FIG. 1.

It is appreciated that a function of VGR418 gene, herein designated VGR GENE, is inhibition of expression of host target genes, as part of a novel viral mechanism of attacking a host. Accordingly, utilities of VGR418 gene include diagnosis, prevention and treatment of viral infection by Vaccinia Virus. Specific functions, and accordingly utilities, of VGR418 gene correlate with, and may be deduced from, the identity of the host target genes, which are inhibited by VGAM RNAs comprised in the ‘operon-like’ cluster of VGR418 gene: VGAM247 host target protein, VGAM248 host target protein, VGAM249 host target protein and VGAM250 host target protein, herein schematically represented by VGAM1 HOST TARGET PROTEIN through VGAM3 HOST TARGET PROTEIN. The function of these host target genes is elaborated hereinabove with reference to VGAM247, VGAM248, VGAM249 and VGAM250. FIG. 9 further provides a conceptual description of novel bioinformatically detected regulatory viral gene, referred to here as Viral Genomic Record 419(VGR419) viral gene, which encodes an ‘operon-like’ cluster of novel viral micro RNA-like genes, each of which in turn modulates expression of at least one host target gene, the function and utility of which at least one host target gene is known in the art.

VGR419 gene, herein designated VGR GENE, is a novel bioinformatically detected regulatory, non protein coding, RNA viral gene. The method by which VGR419 gene was detected is described hereinabove with reference to FIGS. 1-9.

VGR419 gene encodes VGR419 precursor RNA, herein designated VGR PRECURSOR RNA, an RNA molecule, typically several hundred nucleotides long.

VGR419 precursor RNA folds spatially, forming VGR419 folded precursor RNA, herein designated VGR FOLDED PRECURSOR RNA. It is appreciated that VGR419 folded precursor RNA comprises a plurality of what is known in the art as ‘hairpin’ structures. These ‘hairpin’ structures are due to the fact that the nucleotide sequence of VGR419 precursor RNA comprises a plurality of segments, the first half of each such segment having a nucleotide sequence which is at least a partial inversed-reversed sequence of the second half thereof, as is well known in the art.

VGR419 folded precursor RNA is naturally processed by cellular enzymatic activity into at least 3 separate VGAM precursor RNAs, VGAM253 precursor RNA, VGAM254 precursor RNA and VGAM255 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM253 RNA, VGAM254 RNA and VGAM255 RNA, herein schematically represented by VGAM1 RNA through VGAM3 RNA, each of which VGAM RNAs corresponding to VGAM RNA of FIG. 1.

VGAM253 RNA, herein schematically represented by VGAM1 RNA through VGAM3 RNA, binds complementarily to a host target binding site located in an untranslated region of VGAM253 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM253 host target RNA into VGAM253 host target protein, herein schematically represented by VGAM1 HOST TARGET PROTEIN through VGAM3 HOST TARGET PROTEIN, all of FIG. 1.

VGAM254 RNA, herein schematically represented by VGAM1 RNA through VGAM3 RNA, binds complementarily to a host target binding site located in an untranslated region of VGAM254 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM254 host target RNA into VGAM254 host target protein, herein schematically represented by VGAM1 HOST TARGET PROTEIN through VGAM3 HOST TARGET PROTEIN, all of FIG. 1.

VGAM255 RNA, herein schematically represented by VGAM1 RNA through VGAM3 RNA, binds complementarily to a host target binding site located in an untranslated region of VGAM255 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM255 host target RNA into VGAM255 host target protein, herein schematically represented by VGAM1 HOST TARGET PROTEIN through VGAM3 HOST TARGET PROTEIN, all of FIG. 1.

It is appreciated that a function of VGR419 gene, herein designated VGR GENE, is inhibition of expression of host target genes, as part of a novel viral mechanism of attacking a host. Accordingly, utilities of VGR419 gene include diagnosis, prevention and treatment of viral infection by Vaccinia Virus. Specific functions, and accordingly utilities, of VGR419 gene correlate with, and may be deduced from, the identity of the host target genes, which are inhibited by VGAM RNAs comprised in the ‘operon-like’ cluster of VGR419 gene: VGAM253 host target protein, VGAM254 host target protein and VGAM255 host target protein, herein schematically represented by VGAM1 HOST TARGET PROTEIN through VGAM3 HOST TARGET PROTEIN. The function of these host target genes is elaborated hereinabove with reference to VGAM253, VGAM254 and VGAM255. FIG. 9 further provides a conceptual description of novel bioinformatically detected regulatory viral gene, referred to here as Viral Genomic Record 420(VGR420) viral gene, which encodes an ‘operon-like’ cluster of novel viral micro RNA-like genes, each of which in turn modulates expression of at least one host target gene, the function and utility of which at least one host target gene is known in the art.

VGR420 gene, herein designated VGR GENE, is a novel bioinformatically detected regulatory, non protein coding, RNA viral gene. The method by which VGR420 gene was detected is described hereinabove with reference to FIGS. 1-9.

VGR420 gene encodes VGR420 precursor RNA, herein designated VGR PRECURSOR RNA, an RNA molecule, typically several hundred nucleotides long.

VGR420 precursor RNA folds spatially, forming VGR420 folded precursor RNA, herein designated VGR FOLDED PRECURSOR RNA. It is appreciated that VGR420 folded precursor RNA comprises a plurality of what is known in the art as ‘hairpin’ structures. These ‘hairpin’ structures are due to the fact that the nucleotide sequence of VGR420 precursor RNA comprises a plurality of segments, the first half of each such segment having a nucleotide sequence which is at least a partial inversed-reversed sequence of the second half thereof, as is well known in the art.

VGR420 folded precursor RNA is naturally processed by cellular enzymatic activity into at least 2 separate VGAM precursor RNAs, VGAM256 precursor RNA and VGAM257 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM256 RNA and VGAM257 RNA, herein schematically represented by VGAM1 RNA through VGAM3 RNA, each of which VGAM RNAs corresponding to VGAM RNA of FIG. 1.

VGAM256 RNA, herein schematically represented by VGAM1 RNA through VGAM3 RNA, binds complementarily to a host target binding site located in an untranslated region of VGAM256 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM256 host target RNA into VGAM256 host target protein, herein schematically represented by VGAM1 HOST TARGET PROTEIN through VGAM3 HOST TARGET PROTEIN, all of FIG. 1.

VGAM257 RNA, herein schematically represented by VGAM1 RNA through VGAM3 RNA, binds complementarily to a host target binding site located in an untranslated region of VGAM257 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM257 host target RNA into VGAM257 host target protein, herein schematically represented by VGAM1 HOST TARGET PROTEIN through VGAM3 HOST TARGET PROTEIN, all of FIG. 1.

It is appreciated that a function of VGR420 gene, herein designated VGR GENE, is inhibition of expression of host target genes, as part of a novel viral mechanism of attacking a host. Accordingly, utilities of VGR420 gene include diagnosis, prevention and treatment of viral infection by Vaccinia Virus. Specific functions, and accordingly utilities, of VGR420 gene correlate with, and may be deduced from, the identity of the host target genes, which are inhibited by VGAM RNAs comprised in the ‘operon-like’ cluster of VGR420 gene: VGAM256 host target protein and VGAM257 host target protein, herein schematically represented by VGAM1 HOST TARGET PROTEIN through VGAM3 HOST TARGET PROTEIN. The function of these host target genes is elaborated hereinabove with reference to VGAM256 and VGAM257. FIG. 9 further provides a conceptual description of novel bioinformatically detected regulatory viral gene, referred to here as Viral Genomic Record 421(VGR421) viral gene, which encodes an ‘operon-like’ cluster of novel viral micro RNA-like genes, each of which in turn modulates expression of at least one host target gene, the function and utility of which at least one host target gene is known in the art.

VGR421 gene, herein designated VGR GENE, is a novel bioinformatically detected regulatory, non protein coding, RNA viral gene. The method by which VGR421 gene was detected is described hereinabove with reference to FIGS. 1-9.

VGR421 gene encodes VGR421 precursor RNA, herein designated VGR PRECURSOR RNA, an RNA molecule, typically several hundred nucleotides long.

VGR421 precursor RNA folds spatially, forming VGR421 folded precursor RNA, herein designated VGR FOLDED PRECURSOR RNA. It is appreciated that VGR421 folded precursor RNA comprises a plurality of what is known in the art as ‘hairpin’ structures. These ‘hairpin’ structures are due to the fact that the nucleotide sequence of VGR421 precursor RNA comprises a plurality of segments, the first half of each such segment having a nucleotide sequence which is at least a partial inversed-reversed sequence of the second half thereof, as is well known in the art.

VGR421 folded precursor RNA is naturally processed by cellular enzymatic activity into at least 3 separate VGAM precursor RNAs, VGAM263 precursor RNA, VGAM264 precursor RNA and VGAM265 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM263 RNA, VGAM264 RNA and VGAM265 RNA, herein schematically represented by VGAM1 RNA through VGAM3 RNA, each of which VGAM RNAs corresponding to VGAM RNA of FIG. 1.

VGAM263 RNA, herein schematically represented by VGAM1 RNA through VGAM3 RNA, binds complementarily to a host target binding site located in an untranslated region of VGAM263 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM263 host target RNA into VGAM263 host target protein, herein schematically represented by VGAM1 HOST TARGET PROTEIN through VGAM3 HOST TARGET PROTEIN, all of FIG. 1.

VGAM264 RNA, herein schematically represented by VGAM1 RNA through VGAM3 RNA, binds complementarily to a host target binding site located in an untranslated region of VGAM264 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM264 host target RNA into VGAM264 host target protein, herein schematically represented by VGAM1 HOST TARGET PROTEIN through VGAM3 HOST TARGET PROTEIN, all of FIG. 1.

VGAM265 RNA, herein schematically represented by VGAM1 RNA through VGAM3 RNA, binds complementarily to a host target binding site located in an untranslated region of VGAM265 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM265 host target RNA into VGAM265 host target protein, herein schematically represented by VGAM1 HOST TARGET PROTEIN through VGAM3 HOST TARGET PROTEIN, all of FIG. 1.

It is appreciated that a function of VGR421 gene, herein designated VGR GENE, is inhibition of expression of host target genes, as part of a novel viral mechanism of attacking a host. Accordingly, utilities of VGR421 gene include diagnosis, prevention and treatment of viral infection by Vaccinia Virus. Specific functions, and accordingly utilities, of VGR421 gene correlate with, and may be deduced from, the identity of the host target genes, which are inhibited by VGAM RNAs comprised in the ‘operon-like’ cluster of VGR421 gene: VGAM263 host target protein, VGAM264 host target protein and VGAM265 host target protein, herein schematically represented by VGAM1 HOST TARGET PROTEIN through VGAM3 HOST TARGET PROTEIN. The function of these host target genes is elaborated hereinabove with reference to VGAM263, VGAM264 and VGAM265. FIG. 9 further provides a conceptual description of novel bioinformatically detected regulatory viral gene, referred to here as Viral Genomic Record 422(VGR422) viral gene, which encodes an ‘operon-like’ cluster of novel viral micro RNA-like genes, each of which in turn modulates expression of at least one host target gene, the function and utility of which at least one host target gene is known in the art.

VGR422 gene, herein designated VGR GENE, is a novel bioinformatically detected regulatory, non protein coding, RNA viral gene. The method by which VGR422 gene was detected is described hereinabove with reference to FIGS. 1-9.

VGR422 gene encodes VGR422 precursor RNA, herein designated VGR PRECURSOR RNA, an RNA molecule, typically several hundred nucleotides long.

VGR422 precursor RNA folds spatially, forming VGR422 folded precursor RNA, herein designated VGR FOLDED PRECURSOR RNA. It is appreciated that VGR422 folded precursor RNA comprises a plurality of what is known in the art as ‘hairpin’ structures. These ‘hairpin’ structures are due to the fact that the nucleotide sequence of VGR422 precursor RNA comprises a plurality of segments, the first half of each such segment having a nucleotide sequence which is at least a partial inversed-reversed sequence of the second half thereof, as is well known in the art.

VGR422 folded precursor RNA is naturally processed by cellular enzymatic activity into at least 2 separate VGAM precursor RNAs, VGAM266 precursor RNA and VGAM267 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM266 RNA and VGAM267 RNA, herein schematically represented by VGAM1 RNA through VGAM3 RNA, each of which VGAM RNAs corresponding to VGAM RNA of FIG. 1.

VGAM266 RNA, herein schematically represented by VGAM1 RNA through VGAM3 RNA, binds complementarily to a host target binding site located in an untranslated region of VGAM266 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM266 host target RNA into VGAM266 host target protein, herein schematically represented by VGAM1 HOST TARGET PROTEIN through VGAM3 HOST TARGET PROTEIN, all of FIG. 1.

VGAM267 RNA, herein schematically represented by VGAM1 RNA through VGAM3 RNA, binds complementarily to a host target binding site located in an untranslated region of VGAM267 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM267 host target RNA into VGAM267 host target protein, herein schematically represented by VGAM1 HOST TARGET PROTEIN through VGAM3 HOST TARGET PROTEIN, all of FIG. 1.

It is appreciated that a function of VGR422 gene, herein designated VGR GENE, is inhibition of expression of host target genes, as part of a novel viral mechanism of attacking a host. Accordingly, utilities of VGR422 gene include diagnosis, prevention and treatment of viral infection by Vaccinia Virus. Specific functions, and accordingly utilities, of VGR422 gene correlate with, and may be deduced from, the identity of the host target genes, which are inhibited by VGAM RNAs comprised in the ‘operon-like’ cluster of VGR422 gene: VGAM266 host target protein and VGAM267 host target protein, herein schematically represented by VGAM1 HOST TARGET PROTEIN through VGAM3 HOST TARGET PROTEIN. The function of these host target genes is elaborated hereinabove with reference to VGAM266 and VGAM267. FIG. 9 further provides a conceptual description of novel bioinformatically detected regulatory viral gene, referred to here as Viral Genomic Record 423(VGR423) viral gene, which encodes an ‘operon-like’ cluster of novel viral micro RNA-like genes, each of which in turn modulates expression of at least one host target gene, the function and utility of which at least one host target gene is known in the art.

VGR423 gene, herein designated VGR GENE, is a novel bioinformatically detected regulatory, non protein coding, RNA viral gene. The method by which VGR423 gene was detected is described hereinabove with reference to FIGS. 1-9.

VGR423 gene encodes VGR423 precursor RNA, herein designated VGR PRECURSOR RNA, an RNA molecule, typically several hundred nucleotides long.

VGR423 precursor RNA folds spatially, forming VGR423 folded precursor RNA, herein designated VGR FOLDED PRECURSOR RNA. It is appreciated that VGR423 folded precursor RNA comprises a plurality of what is known in the art as ‘hairpin’ structures. These ‘hairpin’ structures are due to the fact that the nucleotide sequence of VGR423 precursor RNA comprises a plurality of segments, the first half of each such segment having a nucleotide sequence which is at least a partial inversed-reversed sequence of the second half thereof, as is well known in the art.

VGR423 folded precursor RNA is naturally processed by cellular enzymatic activity into at least 3 separate VGAM precursor RNAs, VGAM268 precursor RNA, VGAM269 precursor RNA and VGAM270 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM268 RNA, VGAM269 RNA and VGAM270 RNA, herein schematically represented by VGAM1 RNA through VGAM3 RNA, each of which VGAM RNAs corresponding to VGAM RNA of FIG. 1.

VGAM268 RNA, herein schematically represented by VGAM1 RNA through VGAM3 RNA, binds complementarily to a host target binding site located in an untranslated region of VGAM268 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM268 host target RNA into VGAM268 host target protein, herein schematically represented by VGAM1 HOST TARGET PROTEIN through VGAM3 HOST TARGET PROTEIN, all of FIG. 1.

VGAM269 RNA, herein schematically represented by VGAM1 RNA through VGAM3 RNA, binds complementarily to a host target binding site located in an untranslated region of VGAM269 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM269 host target RNA into VGAM269 host target protein, herein schematically represented by VGAM1 HOST TARGET PROTEIN through VGAM3 HOST TARGET PROTEIN, all of FIG. 1.

VGAM270 RNA, herein schematically represented by VGAM1 RNA through VGAM3 RNA, binds complementarily to a host target binding site located in an untranslated region of VGAM270 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM270 host target RNA into VGAM270 host target protein, herein schematically represented by VGAM1 HOST TARGET PROTEIN through VGAM3 HOST TARGET PROTEIN, all of FIG. 1.

It is appreciated that a function of VGR423 gene, herein designated VGR GENE, is inhibition of expression of host target genes, as part of a novel viral mechanism of attacking a host. Accordingly, utilities of VGR423 gene include diagnosis, prevention and treatment of viral infection by Vaccinia Virus. Specific functions, and accordingly utilities, of VGR423 gene correlate with, and may be deduced from, the identity of the host target genes, which are inhibited by VGAM RNAs comprised in the ‘operon-like’ cluster of VGR423 gene: VGAM268 host target protein, VGAM269 host target protein and VGAM270 host target protein, herein schematically represented by VGAM1 HOST TARGET PROTEIN through VGAM3 HOST TARGET PROTEIN. The function of these host target genes is elaborated hereinabove with reference to VGAM268, VGAM269 and VGAM270. FIG. 9 further provides a conceptual description of novel bioinformatically detected regulatory viral gene, referred to here as Viral Genomic Record 424(VGR424) viral gene, which encodes an ‘operon-like’ cluster of novel viral micro RNA-like genes, each of which in turn modulates expression of at least one host target gene, the function and utility of which at least one host target gene is known in the art.

VGR424 gene, herein designated VGR GENE, is a novel bioinformatically detected regulatory, non protein coding, RNA viral gene. The method by which VGR424 gene was detected is described hereinabove with reference to FIGS. 1-9.

VGR424 gene encodes VGR424 precursor RNA, herein designated VGR PRECURSOR RNA, an RNA molecule, typically several hundred nucleotides long.

VGR424 precursor RNA folds spatially, forming VGR424 folded precursor RNA, herein designated VGR FOLDED PRECURSOR RNA. It is appreciated that VGR424 folded precursor RNA comprises a plurality of what is known in the art as ‘hairpin’ structures. These ‘hairpin’ structures are due to the fact that the nucleotide sequence of VGR424 precursor RNA comprises a plurality of segments, the first half of each such segment having a nucleotide sequence which is at least a partial inversed-reversed sequence of the second half thereof, as is well known in the art.

VGR424 folded precursor RNA is naturally processed by cellular enzymatic activity into at least 2 separate VGAM precursor RNAs, VGAM272 precursor RNA and VGAM273 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM272 RNA and VGAM273 RNA, herein schematically represented by VGAM1 RNA through VGAM3 RNA, each of which VGAM RNAs corresponding to VGAM RNA of FIG. 1.

VGAM272 RNA, herein schematically represented by VGAM1 RNA through VGAM3 RNA, binds complementarily to a host target binding site located in an untranslated region of VGAM272 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM272 host target RNA into VGAM272 host target protein, herein schematically represented by VGAM1 HOST TARGET PROTEIN through VGAM3 HOST TARGET PROTEIN, all of FIG. 1.

VGAM273 RNA, herein schematically represented by VGAM1 RNA through VGAM3 RNA, binds complementarily to a host target binding site located in an untranslated region of VGAM273 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM273 host target RNA into VGAM273 host target protein, herein schematically represented by VGAM1 HOST TARGET PROTEIN through VGAM3 HOST TARGET PROTEIN, all of FIG. 1.

It is appreciated that a function of VGR424 gene, herein designated VGR GENE, is inhibition of expression of host target genes, as part of a novel viral mechanism of attacking a host. Accordingly, utilities of VGR424 gene include diagnosis, prevention and treatment of viral infection by Vaccinia Virus. Specific functions, and accordingly utilities, of VGR424 gene correlate with, and may be deduced from, the identity of the host target genes, which are inhibited by VGAM RNAs comprised in the ‘operon-like’ cluster of VGR424 gene: VGAM272 host target protein and VGAM273 host target protein, herein schematically represented by VGAM1 HOST TARGET PROTEIN through VGAM3 HOST TARGET PROTEIN. The function of these host target genes is elaborated hereinabove with reference to VGAM272 and VGAM273. FIG. 9 further provides a conceptual description of novel bioinformatically detected regulatory viral gene, referred to here as Viral Genomic Record 425(VGR425) viral gene, which encodes an ‘operon-like’ cluster of novel viral micro RNA-like genes, each of which in turn modulates expression of at least one host target gene, the function and utility of which at least one host target gene is known in the art.

VGR425 gene, herein designated VGR GENE, is a novel bioinformatically detected regulatory, non protein coding, RNA viral gene. The method by which VGR425 gene was detected is described hereinabove with reference to FIGS. 1-9.

VGR425 gene encodes VGR425 precursor RNA, herein designated VGR PRECURSOR RNA, an RNA molecule, typically several hundred nucleotides long.

VGR425 precursor RNA folds spatially, forming VGR425 folded precursor RNA, herein designated VGR FOLDED PRECURSOR RNA. It is appreciated that VGR425 folded precursor RNA comprises a plurality of what is known in the art as ‘hairpin’ structures. These ‘hairpin’ structures are due to the fact that the nucleotide sequence of VGR425 precursor RNA comprises a plurality of segments, the first half of each such segment having a nucleotide sequence which is at least a partial inversed-reversed sequence of the second half thereof, as is well known in the art.

VGR425 folded precursor RNA is naturally processed by cellular enzymatic activity into at least 4 separate VGAM precursor RNAs, VGAM274 precursor RNA, VGAM275 precursor RNA, VGAM276 precursor RNA and VGAM277 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM274 RNA, VGAM275 RNA, VGAM276 RNA and VGAM277 RNA, herein schematically represented by VGAM1 RNA through VGAM3 RNA, each of which VGAM RNAs corresponding to VGAM RNA of FIG. 1.

VGAM274 RNA, herein schematically represented by VGAM1 RNA through VGAM3 RNA, binds complementarily to a host target binding site located in an untranslated region of VGAM274 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM274 host target RNA into VGAM274 host target protein, herein schematically represented by VGAM1 HOST TARGET PROTEIN through VGAM3 HOST TARGET PROTEIN, all of FIG. 1.

VGAM275 RNA, herein schematically represented by VGAM1 RNA through VGAM3 RNA, binds complementarily to a host target binding site located in an untranslated region of VGAM275 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM275 host target RNA into VGAM275 host target protein, herein schematically represented by VGAM1 HOST TARGET PROTEIN through VGAM3 HOST TARGET PROTEIN, all of FIG. 1.

VGAM276 RNA, herein schematically represented by VGAM1 RNA through VGAM3 RNA, binds complementarily to a host target binding site located in an untranslated region of VGAM276 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM276 host target RNA into VGAM276 host target protein, herein schematically represented by VGAM1 HOST TARGET PROTEIN through VGAM3 HOST TARGET PROTEIN, all of FIG. 1.

VGAM277 RNA, herein schematically represented by VGAM1 RNA through VGAM3 RNA, binds complementarily to a host target binding site located in an untranslated region of VGAM277 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM277 host target RNA into VGAM277 host target protein, herein schematically represented by VGAM1 HOST TARGET PROTEIN through VGAM3 HOST TARGET PROTEIN, all of FIG. 1.

It is appreciated that a function of VGR425 gene, herein designated VGR GENE, is inhibition of expression of host target genes, as part of a novel viral mechanism of attacking a host. Accordingly, utilities of VGR425 gene include diagnosis, prevention and treatment of viral infection by Vaccinia Virus. Specific functions, and accordingly utilities, of VGR425 gene correlate with, and may be deduced from, the identity of the host target genes, which are inhibited by VGAM RNAs comprised in the ‘operon-like’ cluster of VGR425 gene: VGAM274 host target protein, VGAM275 host target protein, VGAM276 host target protein and VGAM277 host target protein, herein schematically represented by VGAM1 HOST TARGET PROTEIN through VGAM3 HOST TARGET PROTEIN. The function of these host target genes is elaborated hereinabove with reference to VGAM274, VGAM275, VGAM276 and VGAM277. FIG. 9 further provides a conceptual description of novel bioinformatically detected regulatory viral gene, referred to here as Viral Genomic Record 426(VGR426) viral gene, which encodes an ‘operon-like’ cluster of novel viral micro RNA-like genes, each of which in turn modulates expression of at least one host target gene, the function and utility of which at least one host target gene is known in the art.

VGR426 gene, herein designated VGR GENE, is a novel bioinformatically detected regulatory, non protein coding, RNA viral gene. The method by which VGR426 gene was detected is described hereinabove with reference to FIGS. 1-9.

VGR426 gene encodes VGR426 precursor RNA, herein designated VGR PRECURSOR RNA, an RNA molecule, typically several hundred nucleotides long.

VGR426 precursor RNA folds spatially, forming VGR426 folded precursor RNA, herein designated VGR FOLDED PRECURSOR RNA. It is appreciated that VGR426 folded precursor RNA comprises a plurality of what is known in the art as ‘hairpin’ structures. These ‘hairpin’ structures are due to the fact that the nucleotide sequence of VGR426 precursor RNA comprises a plurality of segments, the first half of each such segment having a nucleotide sequence which is at least a partial inversed-reversed sequence of the second half thereof, as is well known in the art.

VGR426 folded precursor RNA is naturally processed by cellular enzymatic activity into at least 5 separate VGAM precursor RNAs, VGAM279 precursor RNA, VGAM280 precursor RNA, VGAM281 precursor RNA, VGAM282 precursor RNA and VGAM283 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM279 RNA, VGAM280 RNA, VGAM281 RNA, VGAM282 RNA and VGAM283 RNA, herein schematically represented by VGAM1 RNA through VGAM3 RNA, each of which VGAM RNAs corresponding to VGAM RNA of FIG. 1.

VGAM279 RNA, herein schematically represented by VGAM1 RNA through VGAM3 RNA, binds complementarily to a host target binding site located in an untranslated region of VGAM279 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM279 host target RNA into VGAM279 host target protein, herein schematically represented by VGAM1 HOST TARGET PROTEIN through VGAM3 HOST TARGET PROTEIN, all of FIG. 1.

VGAM280 RNA, herein schematically represented by VGAM1 RNA through VGAM3 RNA, binds complementarily to a host target binding site located in an untranslated region of VGAM280 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM280 host target RNA into VGAM280 host target protein, herein schematically represented by VGAM1 HOST TARGET PROTEIN through VGAM3 HOST TARGET PROTEIN, all of FIG. 1.

VGAM281 RNA, herein schematically represented by VGAM1 RNA through VGAM3 RNA, binds complementarily to a host target binding site located in an untranslated region of VGAM281 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM281 host target RNA into VGAM281 host target protein, herein schematically represented by VGAM1 HOST TARGET PROTEIN through VGAM3 HOST TARGET PROTEIN, all of FIG. 1.

VGAM282 RNA, herein schematically represented by VGAM1 RNA through VGAM3 RNA, binds complementarily to a host target binding site located in an untranslated region of VGAM282 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM282 host target RNA into VGAM282 host target protein, herein schematically represented by VGAM1 HOST TARGET PROTEIN through VGAM3 HOST TARGET PROTEIN, all of FIG. 1.

VGAM283 RNA, herein schematically represented by VGAM1 RNA through VGAM3 RNA, binds complementarily to a host target binding site located in an untranslated region of VGAM283 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM283 host target RNA into VGAM283 host target protein, herein schematically represented by VGAM1 HOST TARGET PROTEIN through VGAM3 HOST TARGET PROTEIN, all of FIG. 1.

It is appreciated that a function of VGR426 gene, herein designated VGR GENE, is inhibition of expression of host target genes, as part of a novel viral mechanism of attacking a host. Accordingly, utilities of VGR426 gene include diagnosis, prevention and treatment of viral infection by Vaccinia Virus. Specific functions, and accordingly utilities, of VGR426 gene correlate with, and may be deduced from, the identity of the host target genes, which are inhibited by VGAM RNAs comprised in the ‘operon-like’ cluster of VGR426 gene: VGAM279 host target protein, VGAM280 host target protein, VGAM281 host target protein, VGAM282 host target protein and VGAM283 host target protein, herein schematically represented by VGAM1 HOST TARGET PROTEIN through VGAM3 HOST TARGET PROTEIN. The function of these host target genes is elaborated hereinabove with reference to VGAM279, VGAM280, VGAM281, VGAM282 and VGAM283. FIG. 9 further provides a conceptual description of novel bioinformatically detected regulatory viral gene, referred to here as Viral Genomic Record 427(VGR427) viral gene, which encodes an ‘operon-like’ cluster of novel viral micro RNA-like genes, each of which in turn modulates expression of at least one host target gene, the function and utility of which at least one host target gene is known in the art.

VGR427 gene, herein designated VGR GENE, is a novel bioinformatically detected regulatory, non protein coding, RNA viral gene. The method by which VGR427 gene was detected is described hereinabove with reference to FIGS. 1-9.

VGR427 gene encodes VGR427 precursor RNA, herein designated VGR PRECURSOR RNA, an RNA molecule, typically several hundred nucleotides long.

VGR427 precursor RNA folds spatially, forming VGR427 folded precursor RNA, herein designated VGR FOLDED PRECURSOR RNA. It is appreciated that VGR427 folded precursor RNA comprises a plurality of what is known in the art as ‘hairpin’ structures. These ‘hairpin’ structures are due to the fact that the nucleotide sequence of VGR427 precursor RNA comprises a plurality of segments, the first half of each such segment having a nucleotide sequence which is at least a partial inversed-reversed sequence of the second half thereof, as is well known in the art.

VGR427 folded precursor RNA is naturally processed by cellular enzymatic activity into at least 4 separate VGAM precursor RNAs, VGAM288 precursor RNA, VGAM289 precursor RNA, VGAM290 precursor RNA and VGAM291 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM288 RNA, VGAM289 RNA, VGAM290 RNA and VGAM291 RNA, herein schematically represented by VGAM1 RNA through VGAM3 RNA, each of which VGAM RNAs corresponding to VGAM RNA of FIG. 1.

VGAM288 RNA, herein schematically represented by VGAM1 RNA through VGAM3 RNA, binds complementarily to a host target binding site located in an untranslated region of VGAM288 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM288 host target RNA into VGAM288 host target protein, herein schematically represented by VGAM1 HOST TARGET PROTEIN through VGAM3 HOST TARGET PROTEIN, all of FIG. 1.

VGAM289 RNA, herein schematically represented by VGAM1 RNA through VGAM3 RNA, binds complementarily to a host target binding site located in an untranslated region of VGAM289 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM289 host target RNA into VGAM289 host target protein, herein schematically represented by VGAM1 HOST TARGET PROTEIN through VGAM3 HOST TARGET PROTEIN, all of FIG. 1.

VGAM290 RNA, herein schematically represented by VGAM1 RNA through VGAM3 RNA, binds complementarily to a host target binding site located in an untranslated region of VGAM290 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM290 host target RNA into VGAM290 host target protein, herein schematically represented by VGAM1 HOST TARGET PROTEIN through VGAM3 HOST TARGET PROTEIN, all of FIG. 1.

VGAM291 RNA, herein schematically represented by VGAM1 RNA through VGAM3 RNA, binds complementarily to a host target binding site located in an untranslated region of VGAM291 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM291 host target RNA into VGAM291 host target protein, herein schematically represented by VGAM1 HOST TARGET PROTEIN through VGAM3 HOST TARGET PROTEIN, all of FIG. 1.

It is appreciated that a function of VGR427 gene, herein designated VGR GENE, is inhibition of expression of host target genes, as part of a novel viral mechanism of attacking a host. Accordingly, utilities of VGR427 gene include diagnosis, prevention and treatment of viral infection by Vaccinia Virus. Specific functions, and accordingly utilities, of VGR427 gene correlate with, and may be deduced from, the identity of the host target genes, which are inhibited by VGAM RNAs comprised in the ‘operon-like’ cluster of VGR427 gene: VGAM288 host target protein, VGAM289 host target protein, VGAM290 host target protein and VGAM291 host target protein, herein schematically represented by VGAM1 HOST TARGET PROTEIN through VGAM3 HOST TARGET PROTEIN. The function of these host target genes is elaborated hereinabove with reference to VGAM288, VGAM289, VGAM290 and VGAM291. FIG. 9 further provides a conceptual description of novel bioinformatically detected regulatory viral gene, referred to here as Viral Genomic Record 428(VGR428) viral gene, which encodes an ‘operon-like’ cluster of novel viral micro RNA-like genes, each of which in turn modulates expression of at least one host target gene, the function and utility of which at least one host target gene is known in the art.

VGR428 gene, herein designated VGR GENE, is a novel bioinformatically detected regulatory, non protein coding, RNA viral gene. The method by which VGR428 gene was detected is described hereinabove with reference to FIGS. 1-9.

VGR428 gene encodes VGR428 precursor RNA, herein designated VGR PRECURSOR RNA, an RNA molecule, typically several hundred nucleotides long.

VGR428 precursor RNA folds spatially, forming VGR428 folded precursor RNA, herein designated VGR FOLDED PRECURSOR RNA. It is appreciated that VGR428 folded precursor RNA comprises a plurality of what is known in the art as ‘hairpin’ structures. These ‘hairpin’ structures are due to the fact that the nucleotide sequence of VGR428 precursor RNA comprises a plurality of segments, the first half of each such segment having a nucleotide sequence which is at least a partial inversed-reversed sequence of the second half thereof, as is well known in the art.

VGR428 folded precursor RNA is naturally processed by cellular enzymatic activity into at least 3 separate VGAM precursor RNAs, VGAM292 precursor RNA, VGAM293 precursor RNA and VGAM294 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1. The above mentioned VGAM precursor RNAs are ‘diced’ by DICER COMPLEX of FIG. 1, yielding respective short RNA segments of about 22 nucleotides in length, VGAM292 RNA, VGAM293 RNA and VGAM294 RNA, herein schematically represented by VGAM1 RNA through VGAM3 RNA, each of which VGAM RNAs corresponding to VGAM RNA of FIG. 1.

VGAM292 RNA, herein schematically represented by VGAM1 RNA through VGAM3 RNA, binds complementarily to a host target binding site located in an untranslated region of VGAM292 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM292 host target RNA into VGAM292 host target protein, herein schematically represented by VGAM1 HOST TARGET PROTEIN through VGAM3 HOST TARGET PROTEIN, all of FIG. 1.

VGAM293 RNA, herein schematically represented by VGAM1 RNA through VGAM3 RNA, binds complementarily to a host target binding site located in an untranslated region of VGAM293 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM293 host target RNA into VGAM293 host target protein, herein schematically represented by VGAM1 HOST TARGET PROTEIN through VGAM3 HOST TARGET PROTEIN, all of FIG. 1.

VGAM294 RNA, herein schematically represented by VGAM1 RNA through VGAM3 RNA, binds complementarily to a host target binding site located in an untranslated region of VGAM294 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM294 host target RNA into VGAM294 host target protein, herein schematically represented by VGAM1 HOST TARGET PROTEIN through VGAM3 HOST TARGET PROTEIN, all of FIG. 1.

It is appreciated that a function of VGR428 gene, herein designated VGR GENE, is inhibition of expression of host target genes, as part of a novel viral mechanism of attacking a host. Accordingly, utilities of VGR428 gene include diagnosis, prevention and treatment of viral infection by Vaccinia Virus. Specific functions, and accordingly utilities, of VGR428 gene correlate with, and may be deduced from, the identity of the host target genes, which are inhibited by VGAM RNAs comprised in the ‘operon-like’ cluster of VGR428 gene: VGAM292 host target protein, VGAM293 host target protein and VGAM294 host target protein, herein schematically represented by VGAM1 HOST TARGET PROTEIN through VGAM3 HOST TARGET PROTEIN. The function of these host target genes is elaborated hereinabove with reference to VGAM292, VGAM293 and VGAM294. FIG. 9 further provides a conceptual description of novel bioinformatically detected regulatory viral gene, referred to here as Viral Genomic Record 429(VGR429) viral gene, which encodes an ‘operon-like’ cluster of novel viral micro RNA-like genes, each of which in turn modulates expression of at least one host target gene, the function and utility of which at least one host target gene is known in the art.

VGR429 gene, herein designated VGR GENE, is a novel bioinformatically detected regulatory, non protein coding, RNA viral gene. The method by which VGR429 gene was detected is described hereinabove with reference to FIGS. 1-9.

VGR429 gene encodes VGR429 precursor RNA, herein designated VGR PRECURSOR RNA, an RNA molecule, typically several hundred nucleotides long.

VGR429 precursor RNA folds spatially, forming VGR429 folded precursor RNA, herein designated VGR FOLDED PRECURSOR RNA. It is appreciated that VGR429 folded precursor RNA comprises a plurality of what is known in the art as ‘hairpin’ structures. These ‘hairpin’ structures are due to the fact that the nucleotide sequence of VGR429 precursor RNA comprises a plurality of segments, the first half of each such segment having a nucleotide sequence which is at least a partial inversed-reversed sequence of the second half thereof, as is well known in the art.

VGR429 folded precursor RNA is naturally processed by cellular enzymatic activity into at least 6 separate VGAM precursor RNAs, VGAM295 precursor RNA, VGAM296 precursor RNA, VGAM297 precursor RNA, VGAM298 precursor RNA, VGAM299 precursor RNA and VGAM300 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM295 RNA, VGAM296 RNA, VGAM297 RNA, VGAM298 RNA, VGAM299 RNA and VGAM300 RNA, herein schematically represented by VGAM1 RNA through VGAM3 RNA, each of which VGAM RNAs corresponding to VGAM RNA of FIG. 1.

VGAM295 RNA, herein schematically represented by VGAM1 RNA through VGAM3 RNA, binds complementarily to a host target binding site located in an untranslated region of VGAM295 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM295 host target RNA into VGAM295 host target protein, herein schematically represented by VGAM1 HOST TARGET PROTEIN through VGAM3 HOST TARGET PROTEIN, all of FIG. 1.

VGAM296 RNA, herein schematically represented by VGAM1 RNA through VGAM3 RNA, binds complementarily to a host target binding site located in an untranslated region of VGAM296 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM296 host target RNA into VGAM296 host target protein, herein schematically represented by VGAM1 HOST TARGET PROTEIN through VGAM3 HOST TARGET PROTEIN, all of FIG. 1.

VGAM297 RNA, herein schematically represented by VGAM1 RNA through VGAM3 RNA, binds complementarily to a host target binding site located in an untranslated region of VGAM297 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM297 host target RNA into VGAM297 host target protein, herein schematically represented by VGAM1 HOST TARGET PROTEIN through VGAM3 HOST TARGET PROTEIN, all of FIG. 1.

VGAM298 RNA, herein schematically represented by VGAM1 RNA through VGAM3 RNA, binds complementarily to a host target binding site located in an untranslated region of VGAM298 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM298 host target RNA into VGAM298 host target protein, herein schematically represented by VGAM1 HOST TARGET PROTEIN through VGAM3 HOST TARGET PROTEIN, all of FIG. 1.

VGAM299 RNA, herein schematically represented by VGAM1 RNA through VGAM3 RNA, binds complementarily to a host target binding site located in an untranslated region of VGAM299 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM299 host target RNA into VGAM299 host target protein, herein schematically represented by VGAM1 HOST TARGET PROTEIN through VGAM3 HOST TARGET PROTEIN, all of FIG. 1.

VGAM300 RNA, herein schematically represented by VGAM1 RNA through VGAM3 RNA, binds complementarily to a host target binding site located in an untranslated region of VGAM300 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM300 host target RNA into VGAM300 host target protein, herein schematically represented by VGAM1 HOST TARGET PROTEIN through VGAM3 HOST TARGET PROTEIN, all of FIG. 1.

It is appreciated that a function of VGR429 gene, herein designated VGR GENE, is inhibition of expression of host target genes, as part of a novel viral mechanism of attacking a host. Accordingly, utilities of VGR429 gene include diagnosis, prevention and treatment of viral infection by Vaccinia Virus. Specific functions, and accordingly utilities, of VGR429 gene correlate with, and may be deduced from, the identity of the host target genes, which are inhibited by VGAM RNAs comprised in the ‘operon-like’ cluster of VGR429 gene: VGAM295 host target protein, VGAM296 host target protein, VGAM297 host target protein, VGAM298 host target protein, VGAM299 host target protein and VGAM300 host target protein, herein schematically represented by VGAM1 HOST TARGET PROTEIN through VGAM3 HOST TARGET PROTEIN. The function of these host target genes is elaborated hereinabove with reference to VGAM295, VGAM296, VGAM297, VGAM298, VGAM299 and VGAM300. FIG. 9 further provides a conceptual description of novel bioinformatically detected regulatory viral gene, referred to here as Viral Genomic Record 430(VGR430) viral gene, which encodes an ‘operon-like’ cluster of novel viral micro RNA-like genes, each of which in turn modulates expression of at least one host target gene, the function and utility of which at least one host target gene is known in the art.

VGR430 gene, herein designated VGR GENE, is a novel bioinformatically detected regulatory, non protein coding, RNA viral gene. The method by which VGR430 gene was detected is described hereinabove with reference to FIGS. 1-9.

VGR430 gene encodes VGR430 precursor RNA, herein designated VGR PRECURSOR RNA, an RNA molecule, typically several hundred nucleotides long.

VGR430 precursor RNA folds spatially, forming VGR430 folded precursor RNA, herein designated VGR FOLDED PRECURSOR RNA. It is appreciated that VGR430 folded precursor RNA comprises a plurality of what is known in the art as ‘hairpin’ structures. These ‘hairpin’ structures are due to the fact that the nucleotide sequence of VGR430 precursor RNA comprises a plurality of segments, the first half of each such segment having a nucleotide sequence which is at least a partial inversed-reversed sequence of the second half thereof, as is well known in the art.

VGR430 folded precursor RNA is naturally processed by cellular enzymatic activity into at least 8 separate VGAM precursor RNAs, VGAM303 precursor RNA, VGAM304 precursor RNA, VGAM305 precursor RNA, VGAM306 precursor RNA, VGAM307 precursor RNA, VGAM308 precursor RNA, VGAM309 precursor RNA and VGAM310 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM303 RNA, VGAM304 RNA, VGAM305 RNA, VGAM306 RNA, VGAM307 RNA, VGAM308 RNA, VGAM309 RNA and VGAM310 RNA, herein schematically represented by VGAM1 RNA through VGAM3 RNA, each of which VGAM RNAs corresponding to VGAM RNA of FIG. 1.

VGAM303 RNA, herein schematically represented by VGAM1 RNA through VGAM3 RNA, binds complementarily to a host target binding site located in an untranslated region of VGAM303 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM303 host target RNA into VGAM303 host target protein, herein schematically represented by VGAM1 HOST TARGET PROTEIN through VGAM3 HOST TARGET PROTEIN, all of FIG. 1.

VGAM304 RNA, herein schematically represented by VGAM1 RNA through VGAM3 RNA, binds complementarily to a host target binding site located in an untranslated region of VGAM304 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM304 host target RNA into VGAM304 host target protein, herein schematically represented by VGAM1 HOST TARGET PROTEIN through VGAM3 HOST TARGET PROTEIN, all of FIG. 1.

VGAM305 RNA, herein schematically represented by VGAM1 RNA through VGAM3 RNA, binds complementarily to a host target binding site located in an untranslated region of VGAM305 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM305 host target RNA into VGAM305 host target protein, herein schematically represented by VGAM1 HOST TARGET PROTEIN through VGAM3 HOST TARGET PROTEIN, all of FIG. 1.

VGAM306 RNA, herein schematically represented by VGAM1 RNA through VGAM3 RNA, binds complementarily to a host target binding site located in an untranslated region of VGAM306 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM306 host target RNA into VGAM306 host target protein, herein schematically represented by VGAM1 HOST TARGET PROTEIN through VGAM3 HOST TARGET PROTEIN, all of FIG. 1.

VGAM307 RNA, herein schematically represented by VGAM1 RNA through VGAM3 RNA, binds complementarily to a host target binding site located in an untranslated region of VGAM307 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM307 host target RNA into VGAM307 host target protein, herein schematically represented by VGAM1 HOST TARGET PROTEIN through VGAM3 HOST TARGET PROTEIN, all of FIG. 1.

VGAM308 RNA, herein schematically represented by VGAM1 RNA through VGAM3 RNA, binds complementarily to a host target binding site located in an untranslated region of VGAM308 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM308 host target RNA into VGAM308 host target protein, herein schematically represented by VGAM1 HOST TARGET PROTEIN through VGAM3 HOST TARGET PROTEIN, all of FIG. 1.

VGAM309 RNA, herein schematically represented by VGAM1 RNA through VGAM3 RNA, binds complementarily to a host target binding site located in an untranslated region of VGAM309 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM309 host target RNA into VGAM309 host target protein, herein schematically represented by VGAM1 HOST TARGET PROTEIN through VGAM3 HOST TARGET PROTEIN, all of FIG. 1.

VGAM310 RNA, herein schematically represented by VGAM1 RNA through VGAM3 RNA, binds complementarily to a host target binding site located in an untranslated region of VGAM310 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM310 host target RNA into VGAM310 host target protein, herein schematically represented by VGAM1 HOST TARGET PROTEIN through VGAM3 HOST TARGET PROTEIN, all of FIG. 1.

It is appreciated that a function of VGR430 gene, herein designated VGR GENE, is inhibition of expression of host target genes, as part of a novel viral mechanism of attacking a host. Accordingly, utilities of VGR430 gene include diagnosis, prevention and treatment of viral infection by Vaccinia Virus. Specific functions, and accordingly utilities, of VGR430 gene correlate with, and may be deduced from, the identity of the host target genes, which are inhibited by VGAM RNAs comprised in the ‘operon-like’ cluster of VGR430 gene: VGAM303 host target protein, VGAM304 host target protein, VGAM305 host target protein, VGAM306 host target protein, VGAM307 host target protein, VGAM308 host target protein, VGAM309 host target protein and VGAM310 host target protein, herein schematically represented by VGAM1 HOST TARGET PROTEIN through VGAM3 HOST TARGET PROTEIN. The function of these host target genes is elaborated hereinabove with reference to VGAM303, VGAM304, VGAM305, VGAM306, VGAM307, VGAM308, VGAM309 and VGAM310. FIG. 9 further provides a conceptual description of novel bioinformatically detected regulatory viral gene, referred to here as Viral Genomic Record 431(VGR431) viral gene, which encodes an ‘operon-like’ cluster of novel viral micro RNA-like genes, each of which in turn modulates expression of at least one host target gene, the function and utility of which at least one host target gene is known in the art.

VGR431 gene, herein designated VGR GENE, is a novel bioinformatically detected regulatory, non protein coding, RNA viral gene. The method by which VGR431 gene was detected is described hereinabove with reference to FIGS. 1-9.

VGR431 gene encodes VGR431 precursor RNA, herein designated VGR PRECURSOR RNA, an RNA molecule, typically several hundred nucleotides long.

VGR431 precursor RNA folds spatially, forming VGR431 folded precursor RNA, herein designated VGR FOLDED PRECURSOR RNA. It is appreciated that VGR431 folded precursor RNA comprises a plurality of what is known in the art as ‘hairpin’ structures. These ‘hairpin’ structures are due to the fact that the nucleotide sequence of VGR431 precursor RNA comprises a plurality of segments, the first half of each such segment having a nucleotide sequence which is at least a partial inversed-reversed sequence of the second half thereof, as is well known in the art.

VGR431 folded precursor RNA is naturally processed by cellular enzymatic activity into at least 4 separate VGAM precursor RNAs, VGAM311 precursor RNA, VGAM312 precursor RNA, VGAM313 precursor RNA and VGAM314 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM311 RNA, VGAM312 RNA, VGAM313 RNA and VGAM314 RNA, herein schematically represented by VGAM1 RNA through VGAM3 RNA, each of which VGAM RNAs corresponding to VGAM RNA of FIG. 1.

VGAM311 RNA, herein schematically represented by VGAM1 RNA through VGAM3 RNA, binds complementarily to a host target binding site located in an untranslated region of VGAM311 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM311 host target RNA into VGAM311 host target protein, herein schematically represented by VGAM1 HOST TARGET PROTEIN through VGAM3 HOST TARGET PROTEIN, all of FIG. 1.

VGAM312 RNA, herein schematically represented by VGAM1 RNA through VGAM3 RNA, binds complementarily to a host target binding site located in an untranslated region of VGAM312 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM312 host target RNA into VGAM312 host target protein, herein schematically represented by VGAM1 HOST TARGET PROTEIN through VGAM3 HOST TARGET PROTEIN, all of FIG. 1.

VGAM313 RNA, herein schematically represented by VGAM1 RNA through VGAM3 RNA, binds complementarily to a host target binding site located in an untranslated region of VGAM313 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM313 host target RNA into VGAM313 host target protein, herein schematically represented by VGAM1 HOST TARGET PROTEIN through VGAM3 HOST TARGET PROTEIN, all of FIG. 1.

VGAM314 RNA, herein schematically represented by VGAM1 RNA through VGAM3 RNA, binds complementarily to a host target binding site located in an untranslated region of VGAM314 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM314 host target RNA into VGAM314 host target protein, herein schematically represented by VGAM1 HOST TARGET PROTEIN through VGAM3 HOST TARGET PROTEIN, all of FIG. 1.

It is appreciated that a function of VGR431 gene, herein designated VGR GENE, is inhibition of expression of host target genes, as part of a novel viral mechanism of attacking a host. Accordingly, utilities of VGR431 gene include diagnosis, prevention and treatment of viral infection by Vaccinia Virus. Specific functions, and accordingly utilities, of VGR431 gene correlate with, and may be deduced from, the identity of the host target genes, which are inhibited by VGAM RNAs comprised in the ‘operon-like’ cluster of VGR431 gene: VGAM311 host target protein, VGAM312 host target protein, VGAM313 host target protein and VGAM314 host target protein, herein schematically represented by VGAM1 HOST TARGET PROTEIN through VGAM3 HOST TARGET PROTEIN. The function of these host target genes is elaborated hereinabove with reference to VGAM311, VGAM312, VGAM313 and VGAM314. FIG. 9 further provides a conceptual description of novel bioinformatically detected regulatory viral gene, referred to here as Viral Genomic Record 432(VGR432) viral gene, which encodes an ‘operon-like’ cluster of novel viral micro RNA-like genes, each of which in turn modulates expression of at least one host target gene, the function and utility of which at least one host target gene is known in the art.

VGR432 gene, herein designated VGR GENE, is a novel bioinformatically detected regulatory, non protein coding, RNA viral gene. The method by which VGR432 gene was detected is described hereinabove with reference to FIGS. 1-9.

VGR432 gene encodes VGR432 precursor RNA, herein designated VGR PRECURSOR RNA, an RNA molecule, typically several hundred nucleotides long.

VGR432 precursor RNA folds spatially, forming VGR432 folded precursor RNA, herein designated VGR FOLDED PRECURSOR RNA. It is appreciated that VGR432 folded precursor RNA comprises a plurality of what is known in the art as ‘hairpin’ structures. These ‘hairpin’ structures are due to the fact that the nucleotide sequence of VGR432 precursor RNA comprises a plurality of segments, the first half of each such segment having a nucleotide sequence which is at least a partial inversed-reversed sequence of the second half thereof, as is well known in the art.

VGR432 folded precursor RNA is naturally processed by cellular enzymatic activity into at least 4 separate VGAM precursor RNAs, VGAM316 precursor RNA, VGAM317 precursor RNA, VGAM318 precursor RNA and VGAM319 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM316 RNA, VGAM317 RNA, VGAM318 RNA and VGAM319 RNA, herein schematically represented by VGAM1 RNA through VGAM3 RNA, each of which VGAM RNAs corresponding to VGAM RNA of FIG. 1.

VGAM316 RNA, herein schematically represented by VGAM1 RNA through VGAM3 RNA, binds complementarily to a host target binding site located in an untranslated region of VGAM316 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM316 host target RNA into VGAM316 host target protein, herein schematically represented by VGAM1 HOST TARGET PROTEIN through VGAM3 HOST TARGET PROTEIN, all of FIG. 1.

VGAM317 RNA, herein schematically represented by VGAM1 RNA through VGAM3 RNA, binds complementarily to a host target binding site located in an untranslated region of VGAM317 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM317 host target RNA into VGAM317 host target protein, herein schematically represented by VGAM1 HOST TARGET PROTEIN through VGAM3 HOST TARGET PROTEIN, all of FIG. 1.

VGAM318 RNA, herein schematically represented by VGAM1 RNA through VGAM3 RNA, binds complementarily to a host target binding site located in an untranslated region of VGAM318 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM318 host target RNA into VGAM318 host target protein, herein schematically represented by VGAM1 HOST TARGET PROTEIN through VGAM3 HOST TARGET PROTEIN, all of FIG. 1.

VGAM319 RNA, herein schematically represented by VGAM1 RNA through VGAM3 RNA, binds complementarily to a host target binding site located in an untranslated region of VGAM319 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM319 host target RNA into VGAM319 host target protein, herein schematically represented by VGAM1 HOST TARGET PROTEIN through VGAM3 HOST TARGET PROTEIN, all of FIG. 1.

It is appreciated that a function of VGR432 gene, herein designated VGR GENE, is inhibition of expression of host target genes, as part of a novel viral mechanism of attacking a host. Accordingly, utilities of VGR432 gene include diagnosis, prevention and treatment of viral infection by Vaccinia Virus. Specific functions, and accordingly utilities, of VGR432 gene correlate with, and may be deduced from, the identity of the host target genes, which are inhibited by VGAM RNAs comprised in the ‘operon-like’ cluster of VGR432 gene: VGAM316 host target protein, VGAM317 host target protein, VGAM318 host target protein and VGAM319 host target protein, herein schematically represented by VGAM1 HOST TARGET PROTEIN through VGAM3 HOST TARGET PROTEIN. The function of these host target genes is elaborated hereinabove with reference to VGAM316, VGAM317, VGAM318 and VGAM319. FIG. 9 further provides a conceptual description of novel bioinformatically detected regulatory viral gene, referred to here as Viral Genomic Record 433(VGR433) viral gene, which encodes an ‘operon-like’ cluster of novel viral micro RNA-like genes, each of which in turn modulates expression of at least one host target gene, the function and utility of which at least one host target gene is known in the art.

VGR433 gene, herein designated VGR GENE, is a novel bioinformatically detected regulatory, non protein coding, RNA viral gene. The method by which VGR433 gene was detected is described hereinabove with reference to FIGS. 1-9.

VGR433 gene encodes VGR433 precursor RNA, herein designated VGR PRECURSOR RNA, an RNA molecule, typically several hundred nucleotides long.

VGR433 precursor RNA folds spatially, forming VGR433 folded precursor RNA, herein designated VGR FOLDED PRECURSOR RNA. It is appreciated that VGR433 folded precursor RNA comprises a plurality of what is known in the art as ‘hairpin’ structures. These ‘hairpin’ structures are due to the fact that the nucleotide sequence of VGR433 precursor RNA comprises a plurality of segments, the first half of each such segment having a nucleotide sequence which is at least a partial inversed-reversed sequence of the second half thereof, as is well known in the art.

VGR433 folded precursor RNA is naturally processed by cellular enzymatic activity into at least 8 separate VGAM precursor RNAs, VGAM320 precursor RNA, VGAM321 precursor RNA, VGAM322 precursor RNA, VGAM323 precursor RNA, VGAM324 precursor RNA, VGAM325 precursor RNA, VGAM326 precursor RNA and VGAM327 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM320 RNA, VGAM321 RNA, VGAM322 RNA, VGAM323 RNA, VGAM324 RNA, VGAM325 RNA, VGAM326 RNA and VGAM327 RNA, herein schematically represented by VGAM1 RNA through VGAM3 RNA, each of which VGAM RNAs corresponding to VGAM RNA of FIG. 1.

VGAM320 RNA, herein schematically represented by VGAM1 RNA through VGAM3 RNA, binds complementarily to a host target binding site located in an untranslated region of VGAM320 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM320 host target RNA into VGAM320 host target protein, herein schematically represented by VGAM1 HOST TARGET PROTEIN through VGAM3 HOST TARGET PROTEIN, all of FIG. 1.

VGAM321 RNA, herein schematically represented by VGAM1 RNA through VGAM3 RNA, binds complementarily to a host target binding site located in an untranslated region of VGAM321 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM321 host target RNA into VGAM321 host target protein, herein schematically represented by VGAM1 HOST TARGET PROTEIN through VGAM3 HOST TARGET PROTEIN, all of FIG. 1.

VGAM322 RNA, herein schematically represented by VGAM1 RNA through VGAM3 RNA, binds complementarily to a host target binding site located in an untranslated region of VGAM322 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM322 host target RNA into VGAM322 host target protein, herein schematically represented by VGAM1 HOST TARGET PROTEIN through VGAM3 HOST TARGET PROTEIN, all of FIG. 1.

VGAM323 RNA, herein schematically represented by VGAM1 RNA through VGAM3 RNA, binds complementarily to a host target binding site located in an untranslated region of VGAM323 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM323 host target RNA into VGAM323 host target protein, herein schematically represented by VGAM1 HOST TARGET PROTEIN through VGAM3 HOST TARGET PROTEIN, all of FIG. 1.

VGAM324 RNA, herein schematically represented by VGAM1 RNA through VGAM3 RNA, binds complementarily to a host target binding site located in an untranslated region of VGAM324 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM324 host target RNA into VGAM324 host target protein, herein schematically represented by VGAM1 HOST TARGET PROTEIN through VGAM3 HOST TARGET PROTEIN, all of FIG. 1.

VGAM325 RNA, herein schematically represented by VGAM1 RNA through VGAM3 RNA, binds complementarily to a host target binding site located in an untranslated region of VGAM325 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM325 host target RNA into VGAM325 host target protein, herein schematically represented by VGAM1 HOST TARGET PROTEIN through VGAM3 HOST TARGET PROTEIN, all of FIG. 1.

VGAM326 RNA, herein schematically represented by VGAM1 RNA through VGAM3 RNA, binds complementarily to a host target binding site located in an untranslated region of VGAM326 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM326 host target RNA into VGAM326 host target protein, herein schematically represented by VGAM1 HOST TARGET PROTEIN through VGAM3 HOST TARGET PROTEIN, all of FIG. 1.

VGAM327 RNA, herein schematically represented by VGAM1 RNA through VGAM3 RNA, binds complementarily to a host target binding site located in an untranslated region of VGAM327 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM327 host target RNA into VGAM327 host target protein, herein schematically represented by VGAM1 HOST TARGET PROTEIN through VGAM3 HOST TARGET PROTEIN, all of FIG. 1.

It is appreciated that a function of VGR433 gene, herein designated VGR GENE, is inhibition of expression of host target genes, as part of a novel viral mechanism of attacking a host. Accordingly, utilities of VGR433 gene include diagnosis, prevention and treatment of viral infection by Vaccinia Virus. Specific functions, and accordingly utilities, of VGR433 gene correlate with, and may be deduced from, the identity of the host target genes, which are inhibited by VGAM RNAs comprised in the ‘operon-like’ cluster of VGR433 gene: VGAM320 host target protein, VGAM321 host target protein, VGAM322 host target protein, VGAM323 host target protein, VGAM324 host target protein, VGAM325 host target protein, VGAM326 host target protein and VGAM327 host target protein, herein schematically represented by VGAM1 HOST TARGET PROTEIN through VGAM3 HOST TARGET PROTEIN. The function of these host target genes is elaborated hereinabove with reference to VGAM320, VGAM321, VGAM322, VGAM323, VGAM324, VGAM325, VGAM326 and VGAM327. FIG. 9 further provides a conceptual description of novel bioinformatically detected regulatory viral gene, referred to here as Viral Genomic Record 434(VGR434) viral gene, which encodes an ‘operon-like’ cluster of novel viral micro RNA-like genes, each of which in turn modulates expression of at least one host target gene, the function and utility of which at least one host target gene is known in the art.

VGR434 gene, herein designated VGR GENE, is a novel bioinformatically detected regulatory, non protein coding, RNA viral gene. The method by which VGR434 gene was detected is described hereinabove with reference to FIGS. 1-9.

VGR434 gene encodes VGR434 precursor RNA, herein designated VGR PRECURSOR RNA, an RNA molecule, typically several hundred nucleotides long.

VGR434 precursor RNA folds spatially, forming VGR434 folded precursor RNA, herein designated VGR FOLDED PRECURSOR RNA. It is appreciated that VGR434 folded precursor RNA comprises a plurality of what is known in the art as ‘hairpin’ structures. These ‘hairpin’ structures are due to the fact that the nucleotide sequence of VGR434 precursor RNA comprises a plurality of segments, the first half of each such segment having a nucleotide sequence which is at least a partial inversed-reversed sequence of the second half thereof, as is well known in the art.

VGR434 folded precursor RNA is naturally processed by cellular enzymatic activity into at least 8 separate VGAM precursor RNAs, VGAM328 precursor RNA, VGAM329 precursor RNA, VGAM330 precursor RNA, VGAM331 precursor RNA, VGAM332 precursor RNA, VGAM333 precursor RNA, VGAM334 precursor RNA and VGAM335 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM328 RNA, VGAM329 RNA, VGAM330 RNA, VGAM331 RNA, VGAM332 RNA, VGAM333 RNA, VGAM334 RNA and VGAM335 RNA, herein schematically represented by VGAM1 RNA through VGAM3 RNA, each of which VGAM RNAs corresponding to VGAM RNA of FIG. 1.

VGAM328 RNA, herein schematically represented by VGAM1 RNA through VGAM3 RNA, binds complementarily to a host target binding site located in an untranslated region of VGAM328 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM328 host target RNA into VGAM328 host target protein, herein schematically represented by VGAM1 HOST TARGET PROTEIN through VGAM3 HOST TARGET PROTEIN, all of FIG. 1.

VGAM329 RNA, herein schematically represented by VGAM1 RNA through VGAM3 RNA, binds complementarily to a host target binding site located in an untranslated region of VGAM329 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM329 host target RNA into VGAM329 host target protein, herein schematically represented by VGAM1 HOST TARGET PROTEIN through VGAM3 HOST TARGET PROTEIN, all of FIG. 1.

VGAM330 RNA, herein schematically represented by VGAM1 RNA through VGAM3 RNA, binds complementarily to a host target binding site located in an untranslated region of VGAM330 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM330 host target RNA into VGAM330 host target protein, herein schematically represented by VGAM1 HOST TARGET PROTEIN through VGAM3 HOST TARGET PROTEIN, all of FIG. 1.

VGAM331 RNA, herein schematically represented by VGAM1 RNA through VGAM3 RNA, binds complementarily to a host target binding site located in an untranslated region of VGAM331 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM331 host target RNA into VGAM331 host target protein, herein schematically represented by VGAM1 HOST TARGET PROTEIN through VGAM3 HOST TARGET PROTEIN, all of FIG. 1.

VGAM332 RNA, herein schematically represented by VGAM1 RNA through VGAM3 RNA, binds complementarily to a host target binding site located in an untranslated region of VGAM332 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM332 host target RNA into VGAM332 host target protein, herein schematically represented by VGAM1 HOST TARGET PROTEIN through VGAM3 HOST TARGET PROTEIN, all of FIG. 1.

VGAM333 RNA, herein schematically represented by VGAM1 RNA through VGAM3 RNA, binds complementarily to a host target binding site located in an untranslated region of VGAM333 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM333 host target RNA into VGAM333 host target protein, herein schematically represented by VGAM1 HOST TARGET PROTEIN through VGAM3 HOST TARGET PROTEIN, all of FIG. 1.

VGAM334 RNA, herein schematically represented by VGAM1 RNA through VGAM3 RNA, binds complementarily to a host target binding site located in an untranslated region of VGAM334 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM334 host target RNA into VGAM334 host target protein, herein schematically represented by VGAM1 HOST TARGET PROTEIN through VGAM3 HOST TARGET PROTEIN, all of FIG. 1.

VGAM335 RNA, herein schematically represented by VGAM1 RNA through VGAM3 RNA, binds complementarily to a host target binding site located in an untranslated region of VGAM335 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM335 host target RNA into VGAM335 host target protein, herein schematically represented by VGAM1 HOST TARGET PROTEIN through VGAM3 HOST TARGET PROTEIN, all of FIG. 1.

It is appreciated that a function of VGR434 gene, herein designated VGR GENE, is inhibition of expression of host target genes, as part of a novel viral mechanism of attacking a host. Accordingly, utilities of VGR434 gene include diagnosis, prevention and treatment of viral infection by Vaccinia Virus. Specific functions, and accordingly utilities, of VGR434 gene correlate with, and may be deduced from, the identity of the host target genes, which are inhibited by VGAM RNAs comprised in the ‘operon-like’ cluster of VGR434 gene: VGAM328 host target protein, VGAM329 host target protein, VGAM330 host target protein, VGAM331 host target protein, VGAM332 host target protein, VGAM333 host target protein, VGAM334 host target protein and VGAM335 host target protein, herein schematically represented by VGAM1 HOST TARGET PROTEIN through VGAM3 HOST TARGET PROTEIN. The function of these host target genes is elaborated hereinabove with reference to VGAM328, VGAM329, VGAM330, VGAM331, VGAM332, VGAM333, VGAM334 and VGAM335. FIG. 9 further provides a conceptual description of novel bioinformatically detected regulatory viral gene, referred to here as Viral Genomic Record 435(VGR435) viral gene, which encodes an ‘operon-like’ cluster of novel viral micro RNA-like genes, each of which in turn modulates expression of at least one host target gene, the function and utility of which at least one host target gene is known in the art.

VGR435 gene, herein designated VGR GENE, is a novel bioinformatically detected regulatory, non protein coding, RNA viral gene. The method by which VGR435 gene was detected is described hereinabove with reference to FIGS. 1-9.

VGR435 gene encodes VGR435 precursor RNA, herein designated VGR PRECURSOR RNA, an RNA molecule, typically several hundred nucleotides long.

VGR435 precursor RNA folds spatially, forming VGR435 folded precursor RNA, herein designated VGR FOLDED PRECURSOR RNA. It is appreciated that VGR435 folded precursor RNA comprises a plurality of what is known in the art as ‘hairpin’ structures. These ‘hairpin’ structures are due to the fact that the nucleotide sequence of VGR435 precursor RNA comprises a plurality of segments, the first half of each such segment having a nucleotide sequence which is at least a partial inversed-reversed sequence of the second half thereof, as is well known in the art.

VGR435 folded precursor RNA is naturally processed by cellular enzymatic activity into at least 5 separate VGAM precursor RNAs, VGAM336 precursor RNA, VGAM337 precursor RNA, VGAM338 precursor RNA, VGAM339 precursor RNA and VGAM340 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM336 RNA, VGAM337 RNA, VGAM338 RNA, VGAM339 RNA and VGAM340 RNA, herein schematically represented by VGAM1 RNA through VGAM3 RNA, each of which VGAM RNAs corresponding to VGAM RNA of FIG. 1.

VGAM336 RNA, herein schematically represented by VGAM1 RNA through VGAM3 RNA, binds complementarily to a host target binding site located in an untranslated region of VGAM336 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM336 host target RNA into VGAM336 host target protein, herein schematically represented by VGAM1 HOST TARGET PROTEIN through VGAM3 HOST TARGET PROTEIN, all of FIG. 1.

VGAM337 RNA, herein schematically represented by VGAM1 RNA through VGAM3 RNA, binds complementarily to a host target binding site located in an untranslated region of VGAM337 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM337 host target RNA into VGAM337 host target protein, herein schematically represented by VGAM1 HOST TARGET PROTEIN through VGAM3 HOST TARGET PROTEIN, all of FIG. 1.

VGAM338 RNA, herein schematically represented by VGAM1 RNA through VGAM3 RNA, binds complementarily to a host target binding site located in an untranslated region of VGAM338 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM338 host target RNA into VGAM338 host target protein, herein schematically represented by VGAM1 HOST TARGET PROTEIN through VGAM3 HOST TARGET PROTEIN, all of FIG. 1.

VGAM339 RNA, herein schematically represented by VGAM1 RNA through VGAM3 RNA, binds complementarily to a host target binding site located in an untranslated region of VGAM339 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM339 host target RNA into VGAM339 host target protein, herein schematically represented by VGAM1 HOST TARGET PROTEIN through VGAM3 HOST TARGET PROTEIN, all of FIG. 1.

VGAM340 RNA, herein schematically represented by VGAM1 RNA through VGAM3 RNA, binds complementarily to a host target binding site located in an untranslated region of VGAM340 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM340 host target RNA into VGAM340 host target protein, herein schematically represented by VGAM1 HOST TARGET PROTEIN through VGAM3 HOST TARGET PROTEIN, all of FIG. 1.

It is appreciated that a function of VGR435 gene, herein designated VGR GENE, is inhibition of expression of host target genes, as part of a novel viral mechanism of attacking a host. Accordingly, utilities of VGR435 gene include diagnosis, prevention and treatment of viral infection by Vaccinia Virus. Specific functions, and accordingly utilities, of VGR435 gene correlate with, and may be deduced from, the identity of the host target genes, which are inhibited by VGAM RNAs comprised in the ‘operon-like’ cluster of VGR435 gene: VGAM336 host target protein, VGAM337 host target protein, VGAM338 host target protein, VGAM339 host target protein and VGAM340 host target protein, herein schematically represented by VGAM1 HOST TARGET PROTEIN through VGAM3 HOST TARGET PROTEIN. The function of these host target genes is elaborated hereinabove with reference to VGAM336, VGAM337, VGAM338, VGAM339 and VGAM340. FIG. 9 further provides a conceptual description of novel bioinformatically detected regulatory viral gene, referred to here as Viral Genomic Record 436(VGR436) viral gene, which encodes an ‘operon-like’ cluster of novel viral micro RNA-like genes, each of which in turn modulates expression of at least one host target gene, the function and utility of which at least one host target gene is known in the art.

VGR436 gene, herein designated VGR GENE, is a novel bioinformatically detected regulatory, non protein coding, RNA viral gene. The method by which VGR436 gene was detected is described hereinabove with reference to FIGS. 1-9.

VGR436 gene encodes VGR436 precursor RNA, herein designated VGR PRECURSOR RNA, an RNA molecule, typically several hundred nucleotides long.

VGR436 precursor RNA folds spatially, forming VGR436 folded precursor RNA, herein designated VGR FOLDED PRECURSOR RNA. It is appreciated that VGR436 folded precursor RNA comprises a plurality of what is known in the art as ‘hairpin’ structures. These ‘hairpin’ structures are due to the fact that the nucleotide sequence of VGR436 precursor RNA comprises a plurality of segments, the first half of each such segment having a nucleotide sequence which is at least a partial inversed-reversed sequence of the second half thereof, as is well known in the art.

VGR436 folded precursor RNA is naturally processed by cellular enzymatic activity into at least 7 separate VGAM precursor RNAs, VGAM341 precursor RNA, VGAM342 precursor RNA, VGAM343 precursor RNA, VGAM344 precursor RNA, VGAM345 precursor RNA, VGAM346 precursor RNA and VGAM347 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM341 RNA, VGAM342 RNA, VGAM343 RNA, VGAM344 RNA, VGAM345 RNA, VGAM346 RNA and VGAM347 RNA, herein schematically represented by VGAM1 RNA through VGAM3 RNA, each of which VGAM RNAs corresponding to VGAM RNA of FIG. 1.

VGAM341 RNA, herein schematically represented by VGAM1 RNA through VGAM3 RNA, binds complementarily to a host target binding site located in an untranslated region of VGAM341 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM341 host target RNA into VGAM341 host target protein, herein schematically represented by VGAM1 HOST TARGET PROTEIN through VGAM3 HOST TARGET PROTEIN, all of FIG. 1.

VGAM342 RNA, herein schematically represented by VGAM1 RNA through VGAM3 RNA, binds complementarily to a host target binding site located in an untranslated region of VGAM342 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM342 host target RNA into VGAM342 host target protein, herein schematically represented by VGAM1 HOST TARGET PROTEIN through VGAM3 HOST TARGET PROTEIN, all of FIG. 1.

VGAM343 RNA, herein schematically represented by VGAM1 RNA through VGAM3 RNA, binds complementarily to a host target binding site located in an untranslated region of VGAM343 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM343 host target RNA into VGAM343 host target protein, herein schematically represented by VGAM1 HOST TARGET PROTEIN through VGAM3 HOST TARGET PROTEIN, all of FIG. 1.

VGAM344 RNA, herein schematically represented by VGAM1 RNA through VGAM3 RNA, binds complementarily to a host target binding site located in an untranslated region of VGAM344 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM344 host target RNA into VGAM344 host target protein, herein schematically represented by VGAM1 HOST TARGET PROTEIN through VGAM3 HOST TARGET PROTEIN, all of FIG. 1.

VGAM345 RNA, herein schematically represented by VGAM1 RNA through VGAM3 RNA, binds complementarily to a host target binding site located in an untranslated region of VGAM345 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM345 host target RNA into VGAM345 host target protein, herein schematically represented by VGAM1 HOST TARGET PROTEIN through VGAM3 HOST TARGET PROTEIN, all of FIG. 1.

VGAM346 RNA, herein schematically represented by VGAM1 RNA through VGAM3 RNA, binds complementarily to a host target binding site located in an untranslated region of VGAM346 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM346 host target RNA into VGAM346 host target protein, herein schematically represented by VGAM1 HOST TARGET PROTEIN through VGAM3 HOST TARGET PROTEIN, all of FIG. 1.

VGAM347 RNA, herein schematically represented by VGAM1 RNA through VGAM3 RNA, binds complementarily to a host target binding site located in an untranslated region of VGAM347 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM347 host target RNA into VGAM347 host target protein, herein schematically represented by VGAM1 HOST TARGET PROTEIN through VGAM3 HOST TARGET PROTEIN, all of FIG. 1.

It is appreciated that a function of VGR436 gene, herein designated VGR GENE, is inhibition of expression of host target genes, as part of a novel viral mechanism of attacking a host. Accordingly, utilities of VGR436 gene include diagnosis, prevention and treatment of viral infection by Vaccinia Virus. Specific functions, and accordingly utilities, of VGR436 gene correlate with, and may be deduced from, the identity of the host target genes, which are inhibited by VGAM RNAs comprised in the ‘operon-like’ cluster of VGR436 gene: VGAM341 host target protein, VGAM342 host target protein, VGAM343 host target protein, VGAM344 host target protein, VGAM345 host target protein, VGAM346 host target protein and VGAM347 host target protein, herein schematically represented by VGAM1 HOST TARGET PROTEIN through VGAM3 HOST TARGET PROTEIN. The function of these host target genes is elaborated hereinabove with reference to VGAM341, VGAM342, VGAM343, VGAM344, VGAM345, VGAM346 and VGAM347. FIG. 9 further provides a conceptual description of novel bioinformatically detected regulatory viral gene, referred to here as Viral Genomic Record 437(VGR437) viral gene, which encodes an ‘operon-like’ cluster of novel viral micro RNA-like genes, each of which in turn modulates expression of at least one host target gene, the function and utility of which at least one host target gene is known in the art.

VGR437 gene, herein designated VGR GENE, is a novel bioinformatically detected regulatory, non protein coding, RNA viral gene. The method by which VGR437 gene was detected is described hereinabove with reference to FIGS. 1-9.

VGR437 gene encodes VGR437 precursor RNA, herein designated VGR PRECURSOR RNA, an RNA molecule, typically several hundred nucleotides long.

VGR437 precursor RNA folds spatially, forming VGR437 folded precursor RNA, herein designated VGR FOLDED PRECURSOR RNA. It is appreciated that VGR437 folded precursor RNA comprises a plurality of what is known in the art as ‘hairpin’ structures. These ‘hairpin’ structures are due to the fact that the nucleotide sequence of VGR437 precursor RNA comprises a plurality of segments, the first half of each such segment having a nucleotide sequence which is at least a partial inversed-reversed sequence of the second half thereof, as is well known in the art.

VGR437 folded precursor RNA is naturally processed by cellular enzymatic activity into at least 8 separate VGAM precursor RNAs, VGAM348 precursor RNA, VGAM349 precursor RNA, VGAM350 precursor RNA, VGAM351 precursor RNA, VGAM352 precursor RNA, VGAM353 precursor RNA, VGAM354 precursor RNA and VGAM355 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM348 RNA, VGAM349 RNA, VGAM350 RNA, VGAM351 RNA, VGAM352 RNA, VGAM353 RNA, VGAM354 RNA and VGAM355 RNA, herein schematically represented by VGAM1 RNA through VGAM3 RNA, each of which VGAM RNAs corresponding to VGAM RNA of FIG. 1.

VGAM348 RNA, herein schematically represented by VGAM1 RNA through VGAM3 RNA, binds complementarily to a host target binding site located in an untranslated region of VGAM348 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM348 host target RNA into VGAM348 host target protein, herein schematically represented by VGAM1 HOST TARGET PROTEIN through VGAM3 HOST TARGET PROTEIN, all of FIG. 1.

VGAM349 RNA, herein schematically represented by VGAM1 RNA through VGAM3 RNA, binds complementarily to a host target binding site located in an untranslated region of VGAM349 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM349 host target RNA into VGAM349 host target protein, herein schematically represented by VGAM1 HOST TARGET PROTEIN through VGAM3 HOST TARGET PROTEIN, all of FIG. 1.

VGAM350 RNA, herein schematically represented by VGAM1 RNA through VGAM3 RNA, binds complementarily to a host target binding site located in an untranslated region of VGAM350 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM350 host target RNA into VGAM350 host target protein, herein schematically represented by VGAM1 HOST TARGET PROTEIN through VGAM3 HOST TARGET PROTEIN, all of FIG. 1.

VGAM351 RNA, herein schematically represented by VGAM1 RNA through VGAM3 RNA, binds complementarily to a host target binding site located in an untranslated region of VGAM351 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM351 host target RNA into VGAM351 host target protein, herein schematically represented by VGAM1 HOST TARGET PROTEIN through VGAM3 HOST TARGET PROTEIN, all of FIG. 1.

VGAM352 RNA, herein schematically represented by VGAM1 RNA through VGAM3 RNA, binds complementarily to a host target binding site located in an untranslated region of VGAM352 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM352 host target RNA into VGAM352 host target protein, herein schematically represented by VGAM1 HOST TARGET PROTEIN through VGAM3 HOST TARGET PROTEIN, all of FIG. 1.

VGAM353 RNA, herein schematically represented by VGAM1 RNA through VGAM3 RNA, binds complementarily to a host target binding site located in an untranslated region of VGAM353 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM353 host target RNA into VGAM353 host target protein, herein schematically represented by VGAM1 HOST TARGET PROTEIN through VGAM3 HOST TARGET PROTEIN, all of FIG. 1.

VGAM354 RNA, herein schematically represented by VGAM1 RNA through VGAM3 RNA, binds complementarily to a host target binding site located in an untranslated region of VGAM354 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM354 host target RNA into VGAM354 host target protein, herein schematically represented by VGAM1 HOST TARGET PROTEIN through VGAM3 HOST TARGET PROTEIN, all of FIG. 1.

VGAM355 RNA, herein schematically represented by VGAM1 RNA through VGAM3 RNA, binds complementarily to a host target binding site located in an untranslated region of VGAM355 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM355 host target RNA into VGAM355 host target protein, herein schematically represented by VGAM1 HOST TARGET PROTEIN through VGAM3 HOST TARGET PROTEIN, all of FIG. 1.

It is appreciated that a function of VGR437 gene, herein designated VGR GENE, is inhibition of expression of host target genes, as part of a novel viral mechanism of attacking a host. Accordingly, utilities of VGR437 gene include diagnosis, prevention and treatment of viral infection by Vaccinia Virus. Specific functions, and accordingly utilities, of VGR437 gene correlate with, and may be deduced from, the identity of the host target genes, which are inhibited by VGAM RNAs comprised in the ‘operon-like’ cluster of VGR437 gene: VGAM348 host target protein, VGAM349 host target protein, VGAM350 host target protein, VGAM351 host target protein, VGAM352 host target protein, VGAM353 host target protein, VGAM354 host target protein and VGAM355 host target protein, herein schematically represented by VGAM1 HOST TARGET PROTEIN through VGAM3 HOST TARGET PROTEIN. The function of these host target genes is elaborated hereinabove with reference to VGAM348, VGAM349, VGAM350, VGAM351, VGAM352, VGAM353, VGAM354 and VGAM355. FIG. 9 further provides a conceptual description of novel bioinformatically detected regulatory viral gene, referred to here as Viral Genomic Record 438(VGR438) viral gene, which encodes an ‘operon-like’ cluster of novel viral micro RNA-like genes, each of which in turn modulates expression of at least one host target gene, the function and utility of which at least one host target gene is known in the art.

VGR438 gene, herein designated VGR GENE, is a novel bioinformatically detected regulatory, non protein coding, RNA viral gene. The method by which VGR438 gene was detected is described hereinabove with reference to FIGS. 1-9.

VGR438 gene encodes VGR438 precursor RNA, herein designated VGR PRECURSOR RNA, an RNA molecule, typically several hundred nucleotides long.

VGR438 precursor RNA folds spatially, forming VGR438 folded precursor RNA, herein designated VGR FOLDED PRECURSOR RNA. It is appreciated that VGR438 folded precursor RNA comprises a plurality of what is known in the art as ‘hairpin’ structures. These ‘hairpin’ structures are due to the fact that the nucleotide sequence of VGR438 precursor RNA comprises a plurality of segments, the first half of each such segment having a nucleotide sequence which is at least a partial inversed-reversed sequence of the second half thereof, as is well known in the art.

VGR438 folded precursor RNA is naturally processed by cellular enzymatic activity into at least 2 separate VGAM precursor RNAs, VGAM356 precursor RNA and VGAM357 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM356 RNA and VGAM357 RNA, herein schematically represented by VGAM1 RNA through VGAM3 RNA, each of which VGAM RNAs corresponding to VGAM RNA of FIG. 1.

VGAM356 RNA, herein schematically represented by VGAM1 RNA through VGAM3 RNA, binds complementarily to a host target binding site located in an untranslated region of VGAM356 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM356 host target RNA into VGAM356 host target protein, herein schematically represented by VGAM1 HOST TARGET PROTEIN through VGAM3 HOST TARGET PROTEIN, all of FIG. 1.

VGAM357 RNA, herein schematically represented by VGAM1 RNA through VGAM3 RNA, binds complementarily to a host target binding site located in an untranslated region of VGAM357 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM357 host target RNA into VGAM357 host target protein, herein schematically represented by VGAM1 HOST TARGET PROTEIN through VGAM3 HOST TARGET PROTEIN, all of FIG. 1.

It is appreciated that a function of VGR438 gene, herein designated VGR GENE, is inhibition of expression of host target genes, as part of a novel viral mechanism of attacking a host. Accordingly, utilities of VGR438 gene include diagnosis, prevention and treatment of viral infection by Vaccinia Virus. Specific functions, and accordingly utilities, of VGR438 gene correlate with, and may be deduced from, the identity of the host target genes, which are inhibited by VGAM RNAs comprised in the ‘operon-like’ cluster of VGR438 gene: VGAM356 host target protein and VGAM357 host target protein, herein schematically represented by VGAM1 HOST TARGET PROTEIN through VGAM3 HOST TARGET PROTEIN. The function of these host target genes is elaborated hereinabove with reference to VGAM356 and VGAM357. FIG. 9 further provides a conceptual description of novel bioinformatically detected regulatory viral gene, referred to here as Viral Genomic Record 439(VGR439) viral gene, which encodes an ‘operon-like’ cluster of novel viral micro RNA-like genes, each of which in turn modulates expression of at least one host target gene, the function and utility of which at least one host target gene is known in the art.

VGR439 gene, herein designated VGR GENE, is a novel bioinformatically detected regulatory, non protein coding, RNA viral gene. The method by which VGR439 gene was detected is described hereinabove with reference to FIGS. 1-9.

VGR439 gene encodes VGR439 precursor RNA, herein designated VGR PRECURSOR RNA, an RNA molecule, typically several hundred nucleotides long.

VGR439 precursor RNA folds spatially, forming VGR439 folded precursor RNA, herein designated VGR FOLDED PRECURSOR RNA. It is appreciated that VGR439 folded precursor RNA comprises a plurality of what is known in the art as ‘hairpin’ structures. These ‘hairpin’ structures are due to the fact that the nucleotide sequence of VGR439 precursor RNA comprises a plurality of segments, the first half of each such segment having a nucleotide sequence which is at least a partial inversed-reversed sequence of the second half thereof, as is well known in the art.

VGR439 folded precursor RNA is naturally processed by cellular enzymatic activity into at least 2 separate VGAM precursor RNAs, VGAM358 precursor RNA and VGAM359 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM358 RNA and VGAM359 RNA, herein schematically represented by VGAM1 RNA through VGAM3 RNA, each of which VGAM RNAs corresponding to VGAM RNA of FIG. 1.

VGAM358 RNA, herein schematically represented by VGAM1 RNA through VGAM3 RNA, binds complementarily to a host target binding site located in an untranslated region of VGAM358 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM358 host target RNA into VGAM358 host target protein, herein schematically represented by VGAM1 HOST TARGET PROTEIN through VGAM3 HOST TARGET PROTEIN, all of FIG. 1.

VGAM359 RNA, herein schematically represented by VGAM1 RNA through VGAM3 RNA, binds complementarily to a host target binding site located in an untranslated region of VGAM359 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM359 host target RNA into VGAM359 host target protein, herein schematically represented by VGAM1 HOST TARGET PROTEIN through VGAM3 HOST TARGET PROTEIN, all of FIG. 1.

It is appreciated that a function of VGR439 gene, herein designated VGR GENE, is inhibition of expression of host target genes, as part of a novel viral mechanism of attacking a host. Accordingly, utilities of VGR439 gene include diagnosis, prevention and treatment of viral infection by Vaccinia Virus. Specific functions, and accordingly utilities, of VGR439 gene correlate with, and may be deduced from, the identity of the host target genes, which are inhibited by VGAM RNAs comprised in the ‘operon-like’ cluster of VGR439 gene: VGAM358 host target protein and VGAM359 host target protein, herein schematically represented by VGAM1 HOST TARGET PROTEIN through VGAM3 HOST TARGET PROTEIN. The function of these host target genes is elaborated hereinabove with reference to VGAM358 and VGAM359. FIG. 9 further provides a conceptual description of novel bioinformatically detected regulatory viral gene, referred to here as Viral Genomic Record 440(VGR440) viral gene, which encodes an ‘operon-like’ cluster of novel viral micro RNA-like genes, each of which in turn modulates expression of at least one host target gene, the function and utility of which at least one host target gene is known in the art.

VGR440 gene, herein designated VGR GENE, is a novel bioinformatically detected regulatory, non protein coding, RNA viral gene. The method by which VGR440 gene was detected is described hereinabove with reference to FIGS. 1-9.

VGR440 gene encodes VGR440 precursor RNA, herein designated VGR PRECURSOR RNA, an RNA molecule, typically several hundred nucleotides long.

VGR440 precursor RNA folds spatially, forming VGR440 folded precursor RNA, herein designated VGR FOLDED PRECURSOR RNA. It is appreciated that VGR440 folded precursor RNA comprises a plurality of what is known in the art as ‘hairpin’ structures. These ‘hairpin’ structures are due to the fact that the nucleotide sequence of VGR440 precursor RNA comprises a plurality of segments, the first half of each such segment having a nucleotide sequence which is at least a partial inversed-reversed sequence of the second half thereof, as is well known in the art.

VGR440 folded precursor RNA is naturally processed by cellular enzymatic activity into at least 2 separate VGAM precursor RNAs, VGAM361 precursor RNA and VGAM362 precursor RNA, herein schematically represented by VGAM1 FOLDED PRECURSOR through VGAM3 FOLDED PRECURSOR, each of which VGAM precursor RNAs being a hairpin shaped RNA segment, corresponding to VGAM FOLDED PRECURSOR RNA of FIG. 1.

The above mentioned VGAM precursor RNAs are ‘diced’ by DICER COMPLEX of FIG. 1, yielding respective short RNA segments of about 22 nucleotides in length, VGAM361 RNA and VGAM362 RNA, herein schematically represented by VGAM1 RNA through VGAM3 RNA, each of which VGAM RNAs corresponding to VGAM RNA of FIG. 1.

VGAM361 RNA, herein schematically represented by VGAM1 RNA through VGAM3 RNA, binds complementarily to a host target binding site located in an untranslated region of VGAM361 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM361 host target RNA into VGAM361 host target protein, herein schematically represented by VGAM1 HOST TARGET PROTEIN through VGAM3 HOST TARGET PROTEIN, all of FIG. 1.

VGAM362 RNA, herein schematically represented by VGAM1 RNA through VGAM3 RNA, binds complementarily to a host target binding site located in an untranslated region of VGAM362 host target RNA, herein schematically represented by VGAM1 HOST TARGET RNA through VGAM3 HOST TARGET RNA, which host target binding site corresponds to a host target binding site such as BINDING SITE I, BINDING SITE II or BINDING SITE III of FIG. 1, thereby inhibiting translation of VGAM362 host target RNA into VGAM362 host target protein, herein schematically represented by VGAM1 HOST TARGET PROTEIN through VGAM3 HOST TARGET PROTEIN, all of FIG. 1.

It is appreciated that a function of VGR440 gene, herein designated VGR GENE, is inhibition of expression of host target genes, as part of a novel viral mechanism of attacking a host. Accordingly, utilities of VGR440 gene include diagnosis, prevention and treatment of viral infection by Vaccinia Virus. Specific functions, and accordingly utilities, of VGR440 gene correlate with, and may be deduced from, the identity of the host target genes, which are inhibited by VGAM RNAs comprised in the ‘operon-like’ cluster of VGR440 gene: VGAM361 host target protein and VGAM362 host target protein, herein schematically represented by VGAM1 HOST TARGET PROTEIN through VGAM3 HOST TARGET PROTEIN. The function of these host target genes is elaborated hereinabove with reference to VGAM361 and VGAM362.

It is appreciated by persons skilled in the art that the present invention is not limited by what has been particularly shown and described hereinabove. Rather the scope of the present invention includes both combinations and subcombinations of the various features described hereinabove as well as variations and modifications which would occur to persons skilled in the art upon reading the specifications and which are not in the prior art.

### BIBLIOGRAPHY

Zuker, C. S.: On the evolution of eyes: would you like it simple or compound? Science 265: 742-743, 1994.
Fishman, G. I.; Moreno, A. P.; Spray, D. C.; Leinwand, L. A.: Functional analysis of human cardiac gap junction channel mutants. Proc. Nat. Acad. Sci. 88: 3525-3529, 1991.
Yamauchi, M.; Yamauchi, N.; Meuth, M.: Molecular cloning of the human CTP synthetase gene by functional complementation with purified human metaphase chromosomes. EMBO J. 9: 2095-2099, 1990.
Vestergaard, P.; Hermann, A. P.; Orskov, H.; Mosekilde, L.; The Danish Osteoporosis Prevention Study: Effect of sex hormone replacement on the insulin-like growth factor system and bone mineral: a cross-sectional and longitudinal study in 595 perimenopausal women participating in the Danish osteoporosis prevention study. J. Clin. Endocr. Metab. 84:2286-2290, 1999.
Shozu, M.; Akasofu, K.; Harada, T.; Kubota, Y.: A new cause of female pseudohermaphroditism: placental aromatase deficiency. J. Clin. Endocr. Metab. 72: 560-566, 1991.
Gorn, A. H.; Rudolph, S. M.; Flannery, M. R.; Morton, C. C.; Weremowicz, S.; Wang, J.-T.; Krane, S. M.; Goldring, S. R.: Expression of two human skeletal calcitonin receptor isoforms cloned from a giant cell tumor of bone. J. Clin. Invest. 95: 2680-2691, 1995.
Bonaiti-Pellie, C.; Briard-Guillemot, M. L.; Feingold, J.; Frezal, J.: Associated congenital malformations in retinoblastoma. Clin. Genet. 7: 37-39, 1975.
Bookstein, R.; Lee, E. Y.-H. P.; To, H.; Young, L.-J.; Sery, T. W.; Hayes, R. C.; Friedmann, T.; Lee, W.-H.: Human retinoblastoma susceptibility gene: genomic organization and analysis of heterozygous intragenic deletion mutants. Proc. Nat. Acad. Sci. 85: 2210-2214, 1988.
Brantley, M. A.; Worley, L.; Harbour, J. W.: Altered expression of Rb and p53 in uveal melanomas following plaque radiotherapy. Am. J. Ophthal. 133: 242-248, 2002.
Bremner, R.; Du, D. C.; Connolly-Wilson, M. J.; Bridge, P.; Ahmad, K. F.; Mostachfi, H.; Rushlow, D.; Dunn, J. M.; Gallie, B. L.: Deletion of RB exons 24 and 25 causes low-penetrance retinoblastoma. Am. J. Hum. Genet. 61: 556-570, 1997.
Briard-Guillemot, M. L.; Bonaiti-Pellie, C.; Feingold, J.; Frezal, J.: Etude genetique du retinoblastome. Human-genetik 24: 271-284, 1974.
Brownstein, S.; de Chadarevian, J.-P.; Little, J. M.: Trilateral retinoblastoma: report of two cases. Arch. Ophthal. 102: 257-262, 1984.
Buchkovich, K.; Duffy, L. A.; Harlow, E.: The retinoblastoma protein is phosphorylated during specific phases of the cell cycle. Cell 58:1097-1105, 1989.
Lohr, G. W.; Waller, H. D.: Zur Biochemie einiger angeborenerhaemolytischer Anaemien. Folia Haemat. 8: 377-397, 1963.
Erneux, C.; Roeckel, N.; Takazawa, K.; Mailleux, P.; Vassart, G.; Mattei, M. G.: Localization of the genes for human inositol 1,4,5-trisphosphate3-kinase A (ITPKA) and B (ITPKB) to chromosome regions 15q14-q21 and 1q41-q43, respectively, by in situ hybridization. Genomics 14:546-547, 1992.
Takazawa, K.; Perret, J.; Dumont, J. E.; Erneux, C.: Molecular cloning and expression of a new putative inositol 1,4,5-trisphosphate3-kinase isoenzyme. Biochem. J. 278: 883-886, 1991.
Bohni, R.; Riesgo-Escovar, J.; Oldham, S.; Brogiolo, W.; Stocker, H.; Andruss, B. F.; Beckingham, K.; Hafen, E.: Autonomous control of cell and organ size by CHICO, a Drosophila homolog of vertebrate IRS1-4. Cell 97: 865-875, 1999.
Fletcher, W. H.; Britz-Cunningham, S. H.; Zuppan, C. W.: Connexin 43 mutations in sporadic and familial defects of laterality. (Letter) New Eng. J. Med. 333: 941-942, 1995.
Gebbia, M.; Towbin, J. A.; Casey, B.: Failure to detect connexin 43 mutations in 38 cases of sporadic and familial heterotaxy. Circulation 94:1909-1912, 1996.
Gebbia, M.; Towbin, J. A.; Casey, B.: Connexin 43 gene mutations and heterotaxy. Response. (Letter) Circulation 97: 118 only, 1998.
Guerrero, P. A.; Schuessler, R. B.; Davis, L. M.; Beyer, E. C.; Johnson, C. M.; Yamada, K. A.; Saffitz, J. E.: Slow ventricular conduction in mice heterozygous for a connexin 43 null mutation. J. Clin. Invest. 99:1991-1998, 1997.
Li, J.-Y.; Hou, X.-E.; Dahlstrom, A.: GAP-43 and its relation to autonomic and sensory neurons in sciatic nerve and gastrocnemius muscle in the rat. J. Auton. Nerv. Syst. 50: 299-309, 1995.
Liao, Y.; Day, K. H.; Damon, D. N.; Duling, B. R.: Endothelial cell-specific knockout of connexin 43 causes hypotension and bradycardia in mice. Proc. Nat. Acad. Sci. 98: 9989-9994, 2001.
Liu, X. Z.; Xia, X. J.; Adams, J.; Chen, Z. Y.; Welch, K. O.; Tekin, M.; Ouyang, X. M.; Kristiansen, A.; Pandya, A.; Balkany, T.; Arnos, K. S.; Nance, W. E.: Mutations in GJA1 (connexin 43) are associated with non-syndromic autosomal recessive deafness. Hum. Molec. Genet. 10:2945-2951, 2001.
Reaume, A. G.; de Sousa, P. A.; Kulkarni, S.; Langille, B. L.; Zhu, D.; Davies, T. C.; Juneja, S. C.; Kidder, G. M.; Rossant, J.: Cardiac malformation in neonatal mice lacking connexin 43. Science 267:1831-1834, 1995.
Splitt, M. P.; Burn, J.; Goodship, J.: Connexin 43 mutations insporadic and familial defects of laterality. (Letter) New Eng. J. Med. 333: 941, 1995.
Splitt, M. P.; Tsai, M. Y.; Burn, J.; Goodship, J. A.: Absence of mutations in the regulatory domain of the gap junction protein connexin 43 in patients with visceroatrial heterotaxy. Heart 77:369-370, 1997.
Toth, T.; Hajdu, J.; Marton, T.; Nagy, B.; Papp, Z.: Connexin 43 gene mutations and heterotaxy. (Letter) Circulation 97: 117-118, 1998.
Ya, J.; Erdstieck-Ernste, E. B. H. W.; de Boer, P. A. J.; van Kempen, M. J. A.; Jongsma, H.; Gros, D.; Moorman, A. F. M.; Lamers, W. H.: Heart defects in connexin 43-deficient mice. Circ. Res. 82:360-366, 1998.
Harbour, J. W.; Luo, R. X.; Dei Santi, A.; Postigo, A. A.; Dean, D. C.: Cdk phosphorylation triggers sequential intramolecular interactions that progressively block Rb functions as cells move through G1. Cell 98:859-869, 1999.
Bullrich, F.; MacLachlan, T. K.; Sang, N.; Druck, T.; Veronese, M. L.; Allen, S. L.; Chiorazzi, N.; Koff, A.; Heubner, K.; Croce, C. M.; Giordano, A.: Chromosomal mapping of members of the cdc2 family of protein kinases, cdk3, cdk6, PISSLRE, and PITALRE, and a cdk inhibitor, p27-Kip1, to regions involved in human cancer. Cancer Res. 55: 1199-1205, 1995.
Abraham, J. A.; Mergia, A.; Whang, J. L.; Tumolo, A.; Friedman, J.; Hjerild, K. A.; Gospodarowicz, D.; Fiddes, J. C.: Nucleotide sequence of a bovine clone encoding the angiogenic protein, basic fibroblast growth factor. Science 233: 545-548, 1986.
Abraham, J. A.; Whang, J. L.; Tumolo, A.; Mergia, A.; Friedman, J.; Gospodarowicz, D.; Fiddes, J. C.: Human basic fibroblast growth factor: nucleotide sequence and genomic organization. EMBO J. 5:2523-2528, 1986.
Doniach, T.: Basic FGF as an inducer of anteroposterior neural pattern. Cell 83: 1067-1070, 1995.
Dono, R.; Texido, G.; Dussel, R.; Ehmke, H.; Zeller, R.: impaired cerebral cortex development and blood pressure regulation in FGF2-deficient mice. EMBO J. 17: 4213-4225, 1998.
Fukushima, Y.; Byers, M. G.; Fiddes, J. C.; Shows, T. B.: The human basic fibroblast growth factor gene (FGFB) is assigned to chromosome 4q25. Cytogenet. Cell Genet. 54: 159-160, 1990.
Gritti, A.; Parati, E. A.; Cova, L.; Frolichsthal, P.; Galli, R.; Wanke, E.; Faravelli, L.; Morassutti, D. J.; Roisen, F.; Nickel, D. D.; Vescovi, A. L.: Multipotential stem cells from the adult mouse brain proliferate and self-renew in response to basic fibroblast growth factor. J. Neurosci. 16: 1091-1100, 1996.
Kawaguchi, H.; Nakamura, K.; Tabata, Y.; Ikada, Y.; Aoyama, I.; Anzai, J.; Nakamura, T.; Hiyama, Y.; Tamura, M.: Acceleration of fracture healing in nonhuman primates by fibroblast growth factor-2. J. Clin. Endocr. Metab. 86: 875-880, 2001.
Kurokawa, T.; Sasada, R.; Iwane, M.; Igarashi, K.: Cloning and expression of cDNA encoding human basic fibroblast growth factor. FEBS Lett. 213: 189-194, 1987.
Lafage-Pochitaloff, M.; Galland, F.; Simonetti, J.; Prats, H.; Mattei, M.-G.; Birnbaum, D.: The human basic fibroblast growth factor gene is located on the long arm of chromosome 4 at bands q26-q27. Oncogene Res. 5: 241-244, 1990.
Mattei, M.-G.; Pebusque, M.-J.; Birnbaum, D.: Chromosomal localizations of mouse Fgf2 and Fgf5 genes. Mammalian Genome 2: 135-137, 1992.
Montero, A.; Okada, Y.; Tomita, M.; Ito, M.; Tsurukami, H.; Nakamura, T.; Doetschman, T.; Coffin, J. D.; Hurley, M. M.: Disruption of the fibroblast growth factor-2 gene results in decreased bone mass and bone formation. J. Clin. Invest. 105: 1085-1093, 2000.
Ortega, S.; Ittmann, M.; Tsang, S. H.; Ehrlich, M.; Basilico, C.: Neuronal defects and delayed wound healing in mice lacking fibroblast growth factor 2. Proc. Nat. Acad. Sci. 95: 5672-5677, 1998.
Plotnikov, A. N.; Schlessinger, J.; Hubbard, S. R.; Mohammadi, M.: Structural basis for FGF receptor dimerization and activation. Cell 98:641-650, 1999
Avraham, K. B.; Givol, D.; Avivi, A.; Yayon, A.; Copeland, N. G.; Jenkins, N. A.: Mapping of murine fibroblast growth factor receptors refines regions of homology between mouse and human chromosomes. Genomics 21:656-658, 1994.
Szepetowski, P.; Perucca-Lostanlen, D.; Gaudray, P.: Mapping genes according to their amplification status in tumor cells: contribution to the map of 11q13. Genomics 16: 745-750, 1993.
Schlossman, S. F.; Boumsell, L.; Gilks, W.; Harlan, J. M.; Kishimoto, T.; Morimoto, C.; Ritz, J.; Shaw, S.; Silverstein, R. L.; Springer, T. A.; Tedder, T. F.; Todd, R. F.: CD antigens 1993. Immun. Today 15:98-99, 1994.
Whitehouse, D. B.; Attwood, J.; Green, C.; Bruce, M.; McQuade, M.; Tippett, P.: Inheritance and linkage data for an unusual combination of genes (at the LKE, P1 and C6 loci) in a single large sibship. Ann. Hum. Genet. 52: 197-201, 1988.
Daniels, G. L.; Le Pennec, P. Y.; Rouger, P.; Salmon, C.; Tippett, P.: The red cell antigens Au(a) and Au(b) belong to the Lutheran system. Vox Sang. 60: 191-192, 1991.
Campbell, I. G.; Foulkes, W. D.; Senger, G.; Trowsdale, J.; Garin-Chesa, P.; Rettig, W. J.: Molecular cloning of the B-CAM cell surface glycoprotein of epithelial cancers: a novel member of the immunoglobulin super family. Cancer Res. 54: 5761-5765, 1994.
Cook, P. J. L.: The Lutheran-secretor recombination fraction in man: a possible sex difference. Ann. Hum. Genet. 28: 393-401, 1965.
Higashiyama, S.; Lau, K.; Besner, G. E.; Abraham, J. A.; Klagsbrun, M.: Structure of heparin-binding EGF-like growth factor: multiple forms, primary structure, and glycosylation of the mature protein. J. Biol. Chem. 267: 6205-6212, 1992.
Naglich, J. G.; Metherall, J. E.; Russell, D. W.; Eidels, L.: Expression cloning of a diphtheria toxin receptor: identity with a heparin-binding EGF-like growth factor precursor. Cell 69: 1051-1061, 1992.
Pappenheimer, A. M., Jr.: Diphtheria toxin. Ann. Rev. Biochem. 46:69-94, 1977.
Pappenheimer, A. M., Jr.; Gill, D. M.: Diphtheria. Science 182:353-358, 1973.
Pathak, B. G.; Gilbert, D. J.; Harrison, C. A.; Luetteke, N. C.; Chen, X.; Klagsbrun, M.; Plowman, G. D.; Copeland, N. G.; Jenkins, N. A.; Lee, D. C.: Mouse chromosomal location of three EGF receptor ligands: amphiregulin (Areg), beta-cellulin (Btc), and heparin-binding EGF (Hegfl). Genomics 28: 116-118, 1995.
Roberts, M.; Ruddle, F. H.: The Chinese hamster gene map: assignment of four genes (DTS, PGM2, 6PGD, Eno1) to chromosome 2. Exp. Cell Res. 127: 47-54, 1980.
Keegan, K.; Johnson, D. E.; Williams, L. T.; Hayman, M. J.: Isolation of an additional member of the fibroblast growth factor receptor family, FGFR-3. Proc. Nat. Acad. Sci. 88: 1095-1099, 1991.
Jutel, M.; Watanabe, T.; Klunker, S.; Akdis, M.; Thomet, O. A. R.; Malolepszy, J.; Zak-Nejmark, T.; Koga, R.; Kobayashi, T.; Blaser, K.; Akdis, C. A.: Histamine regulates T-cell and antibody responses by differential expression of H1 and H2 receptors. Nature 413: 420-425, 2001.
Dixon, M. J.; Gazzard, J.; Chaudhry, S. S.; Sampson, N.; Schulte, B. A.; Steel, K. P.: Mutation of the Na—K—Cl cotransporter gene Slc12a2 results in deafness in mice. Hum. Molec. Genet. 8: 1579-1584, 1999.
Bora, N. S.; Lublin, D. M.; Kumar, B. V.; Hockett, R. D.; Holers, V. M.; Atkinson, J. P.: Structural gene for human membrane cofactor protein (MCP) of complement maps to within 100 kb of the 3-prime end of the C3b/C4b receptor gene. J. Exp. Med. 169: 597-602, 1989
Cui, W.; Hourcade, D.; Post, T.; Greenlund, A. C.; Atkinson, J. P.; Kumar, V.: Characterization of the promoter region of the membrane cofactor protein (CD46) gene of the human complement system and comparison to a membrane cofactor protein-like genetic element. J. Immun. 151:4137-4146, 1993.
Dorig, R. E.; Marcil, A.; Chopra, A.; Richardson, C. D.: The human CD46 molecule is a receptor for measles virus (Edmonston strain). Cell 75:295-305, 1993.
Kallstrom, H.; Gill, D. B.; Albiger, B.; Liszewski, M. K.; Atkinson, J. P.; Jonsson, A.-B.: Attachment of Neisseria gonorrhoeae to the cellular pilus receptor CD46: identification of domains important for bacterial adherence. Cell. Microbiol. 3: 133-143, 2001.
Lublin, D. M.; Liszewski, M. K.; Post, T. W.; Arce, M. A.; LeBeau, M. M.; Rebentisch, M. B.; Lemons, R. S.; Seya, T.; Atkinson, J. P.: Molecular cloning and chromosomal localization of human complement membrane cofactor protein (MCP): evidence for inclusion in the multigene family of complement-regulatory proteins. J. Exp. Med. 168: 181-194, 1988.
Marie, J. C.; Astier, A. L.; Rivailler, P.; Rabourdin-Combe, C.; Wild, T. F.; Horvat, B.: Linking innate and acquired immunity: divergent role of CD46 cytoplasmic domains in T cell-induced inflammation. Nature Immun. 3: 659-666, 2002.
Post, T. W.; Liszewski, M. K.; Adams, E. M.; Tedja, I.; Miller, E. A.; Atkinson, J. P.: Membrane cofactor protein of the complement system: alternative splicing of serine/threonine/proline-rich exons and cytoplasmic tails produces multiple isoforms that correlate with protein phenotype. J. Exp. Med. 174: 93-102, 1991.
Purcell, D. F. J.; Johnstone, R. W.; McKenzie, I. F. C.: Identification of four different CD46 (MCP) molecules with antipeptide antibodies. Biochem. Biophys. Res. Commun. 180: 1091-1097, 1991.
Santoro, F.; Kennedy, P. E.; Locatelli, G.; Malnati, M. S.; Berger, E. A.; Lusso, P.: CD46 is a cellular receptor for human herpes virus 6. Cell 99: 817-827, 1999.
Tatsuo, H.; Ono, N.; Tanaka, K.; Yanagi, Y.: SLAM (CDw150) is a cellular receptor for measles virus. Nature 406: 893-897, 2000.
Kramer, A.; Yang, F.-C.; Snodgrass, P.; Li, X.; Scammell, T. E.; Davis, F. C.; Weitz, C. J.: Regulation of daily locomotor activity and sleep by hypothalamic EGF receptor signaling. Science 294: 2511-2515, 2001
Lanzetti, L.; Rybin, V.; Malabarba, M. G.; Christoforidis, S.; Scita, G.; Zerial, M.; Di Fiore, P. P.: The Eps8 protein coordinates EGF receptor signalling through Rac and trafficking through Rab5. Nature 408:374-377, 2000.
Volpert, O. V.; Zaichuk, T.; Zhou, W.; Reiher, F.; Ferguson, T. A.; Stuart, P. M.; Amin, M.; Bouck, N. P.: Inducer-stimulated Fas targets activated endothelium for destruction by anti-angiogenic thrombospondin-1 and pigment epithelium-derived factor. Nature Med. 8: 349-357, 2002.
Yang, J.; Patil, R. V.; Yu, H.; Gordon, M.; Wax, M. B.: T cell-subsets and sIL-2R/IL-2 levels in patients with glaucoma. Am. J. Ophthal. 131: 421-426, 2001.
Cenciarelli, C.; Chiaur, D. S.; Guardavaccaro, D.; Parks, W.; Vidal, M.; Pagano, M.: Identification of a family of human F-box proteins. Curr. Biol. 9: 1177-1179, 1999.
Winston, J. T.; Koepp, D. M.; Zhu, C.; Elledge, S. J.; Harper, J. W.: A family of mammalian F-box proteins. Curr. Biol. 9: 1180-1182, 1999.
Mullis, P. E.; Patel, M. S.; Brickell, P. M.; Hindmarsh, P. C.; Brook, C. G. D.: Growth characteristics and response to growth hormone therapy in patients with hypochondroplasia: genetic linkage of the insulin-like growth factor I gene at chromosome 12q23 to the disease in a subgroup of these patients. Clin. Endocr. 34: 265-274, 1991.
Delespesse, G.; Sarfati, M.; Peleman, R.: Influence of recombinant IL-4, IFN-alpha, and IFN-gamma on the production of human IgE-binding factor (soluble CD23). J. Immun. 142: 134-138, 1989.
Ludin, C.; Hofstetter, H.; Sarfati, M.; Levy, C. A.; Suter, U.; Alaimo, D.; Kilchherr, E.; Frost, H.; Delespesse, G.: Cloning and expression of the cDNA coding for a human lymphocyte IgE receptor. EMBO J. 6: 109-114, 1987.
Wendel-Hansen, V.; Riviere, M.; Uno, M.; Jansson, I.; Szpirer, J.; Islam, M. Q.; Levan, G.; Klein, G.; Yodoi, J.; Rosen, A.; Szpirer, C.: The gene encoding CD23 leukocyte antigen (FCE2) is located on human chromosome 19. Somat. Cell Molec. Genet. 16: 283-286, 1990.
Lai, L.; Hart, I.; Patterson, D.: Human chromosome 1 corrects the defect in the CHO mutant (Ade-H) deficient in a branch point enzyme in purine de novo biosynthesis. (Abstract) Cytogenet. Cell Genet. 51:1028 only, 1989.
Lai, L.-W.; Hart, I. M.; Patterson, D.: A gene correcting the defect in the CHO mutant Ade(−)H, deficient in a branch point enzyme (adenylosuccinate synthetase) of de novo purine biosynthesis, is located on the long arm of chromosome 1. Genomics 9: 322-328, 1991
Powell, S. M.; Zalkin, H.; Dixon, J. E.: Cloning and characterization of the cDNA encoding human adenylosuccinate synthetase. FEBS Lett. 303:4-10, 1992.
Stengel, D.; Parma, J.; Gannage, M.-H.; Roeckel, N.; Mattei, M.-G.; Barouki, R.; Hanoune, J.: Different chromosomal localization of two adenylyl cyclase genes expressed in human brain. Hum. Genet. 90:126-130, 1992.
Edelhoff, S.; Villacres, E. C.; Storm, D. R.; Disteche, C. M.: Mapping of adenylyl cyclase genes type I, II, III, IV, V, and VI in mouse. Mammalian Genome 6: 111-113, 1995.
Gaudin, C.; Homcy, C. J.; Ishikawa, Y.: Mammalian adenylyl cyclase family members are randomly located on different chromosomes. Hum. Genet. 94: 527-529, 1994.
Wong, S. T.; Trinh, K.; Hacker, B.; Chan, G. C. K.; Lowe, G.; Gaggar, A.; Xia, Z.; Gold, G. H.; Storm, D. R.: Disruption of the type III adenylyl cyclase gene leads to peripheral and behavioral anosmia in transgenic mice. Neuron 27: 487-497, 2000.
E1 Nemer, W.; Rahuel, C.; Colin, Y.; Gane, P.; Cartron, J. P.; Le Van Kim, C.: Organization of the human LU gene and molecular basis of the Lu(a)/Lu(b) blood group polymorphism. Blood 89: 4608-4616, 1997.
Frandson, S.; Atkins, C. J.; Moulds, M.; Poole, J.; Crawford, M. N.; Tippett, P.: Anti-Au(b): the antithetical antibody to anti-Au(a). Vox Sang. 56: 54-56, 1989.
Lewis, M.; Kaita, H.; Chown, B.; Giblett, E. R.; Anderson, J.; Cote, G. B.: The Lutheran and Secretor loci: genetic linkage analysis. Am. J. Hum. Genet. 29: 101-106, 1977.
Lewis, M.; Kaita, H.; Coghlan, G.; Philipps, S.; Belcher, E.; McAlpine, P. J.; Coopland, G. R.; Woods, R. A.: The chromosome 19 linkage group LDLR, C3, LW, APOC2, LU, SE in man. Ann. Hum. Genet. 52: 137-144, 1988.
Lewis, M.; Kaita, H.; Giblett, E. R.; Anderson, J. E.: Lods for Lu:Se and other loci. Cytogenet. Cell Genet. 22: 627-628, 1978.
Mohr, J.: Search for linkage between Lutheran blood group and other hereditary characters. Acta Path. Microbiol. Scand. 28: 207-210, 1951.
Parsons, S. F.; Lee, G.; Spring, F. A.; Willig, T.-N.; Peters, L. L.; Gimm, J. A.; Tanner, M. J. A.; Mohandas, N.; Anstee, D. J.; Chasis, J. A.: Lutheran blood group glycoprotein and its newly characterized mouse homologue specifically bind alpha-5 chain-containing human laminin with high affinity. Blood 97: 312-320, 2001.
Parsons, S. F.; Mallinson, G.; Daniels, G. L.; Green, C. A.; Smythe, J. S.; Anstee, D. J.: Use of domain-deletion mutants to locate Lutheran blood group antigens to each of the five immunoglobulin super family domains of the Lutheran glycoprotein: elucidation of the molecular basis of the Lu(a)/Lu(b) and the Au(a)/Au(b) polymorphisms. Blood 89:4219-4225, 1997.
Parsons, S. F.; Mallinson, G.; Holmes, C. H.; Houlihan, J. M.; Simpson, K. L.; Mawby, W. J.; Spurr, N. K.; Warne, D.; Barclay, A. N.; Anstee, D. J.: The Lutheran blood group glycoprotein, another member of the immunoglobulin super family, is widely expressed in human tissues and is developmentally regulated in human liver. Proc. Nat. Acad. Sci. 92: 5496-5500, 1995.
Rahuel, C.; Le Van Kim, C.; Mattei, M. G.; Cartron, J. P.; Colin, Y.: A unique gene encodes splice forms of the B-cell adhesion molecule cell surface glycoprotein of epithelial cancer and of the Lutheran blood group glycoprotein. Blood 88: 1865-1872, 1996.
Salmon, C.; Salmon, D.; Liberge, G.; Andre, R.; Tippett, P.; Sanger, R.: Un nouvel antigene de groupe sanguin erythrocytaire present chez 80% des sujets de race blanche. Nouv. Rev. Franc. Hemat. 1: 649-661, 1961.
Zelinski, T.; Kaita, H.; Johnson, K.; Moulds, M.: Genetic evidence that the gene controlling Au(b) is located on chromosome 19. Vox Sang. 58: 126-128, 1990.
Evans, R. M.: The steroid and thyroid hormone receptor super family. Science 240:889-895, 1988.
Bader, B. L.; Rayburn, H.; Crowley, D.; Hynes, R. O.: Extensive vasculogenesis, angiogenesis, and organogenesis precede lethality in mice lacking all alpha-V integrins. Cell 95: 507-519, 1998.
Fernandez-Ruiz, E.; de Villena, F. P.-M.; Rodriguez de Cordoba, S.; Sanchez-Madrid, F.: Regional localization of the human vitronectin receptor alpha-subunit gene (VNRA) to chromosome 2q31-q32. Cytogenet. Cell Genet. 62: 26-28, 1993.
Sims, M. A.; Field, S. D.; Barnes, M. R.; Shaikh, N.; Ellington, K.; Murphy, K. E.; Spurr, N.; Campbell, D. A.: Cloning and characterisation of ITGAV, the genomic sequence for human cell adhesion protein (vitronectin) receptor alpha subunit, CD51. Cytogenet. Cell Genet. 89: 268-271, 2000.
Suzuki, S.; Argraves, W. S.; Pytela, R.; Arai, H.; Krusius, T.; Pierschbacher, M. D.; Ruoslahti, E.: cDNA and amino acid sequences of the cell adhesion protein receptor recognizing vitronectin reveal a transmembrane domain and homologies with other adhesion protein receptors. Proc. Nat. Acad. Sci. 83: 8614-8618, 1986
Hromas, R.; Collins, S. J.; Hickstein, D.; Raskind, W.; Deaven, L. L.; O'Hara, P.; Hagen, F. S.; Kaushansky, K.: A retinoic acid-responsive human zinc finger gene, MZF-1, preferentially expressed in myeloid cells. J. Biol. Chem. 266: 14183-14187, 1991.
Morris, J. F.; Rauscher, F. J., III; Davis, B.; Klemsz, M.; Xu, D.; Tenen, D.; Hromas, R.: The myeloid zinc finger gene, MZF-1, regulates the CD34 promoter in vitro. Blood 86: 3640-3647, 1995.
Tommerup, N.; Aagaard, L.; Lund, C. L.; Boel, E.; Baxendale, S.; Bates, G. P.; Lehrach, H.; Vissing, H.: A zinc-finger gene ZNF141 mapping at 4p16.3/D4S90 is a candidate gene for the Wolf-Hirschhorn(4p-) syndrome. Hum. Molec. Genet. 2: 1571-1575, 1993.
de Silva, H. V.; Harmony, J. A. K.; Stuart, W. D.; Gil, C. M.; Robbins, J.: Apolipoprotein J: structure and tissue distribution. Biochemistry 29:5380-5389, 1990.
de Silva, H. V.; Stuart, W. D.; Park, Y. B.; Mao, S. J. T.; Gil, C. M.; Wetterau, J. R.; Busch, S. J.; Harmony, J. A. K.: Purification and characterization of apolipoprotein J. J. Biol. Chem. 265: 14292-14297, 1990.
Dietzsch, E.; Murphy, B. F.; Kirszbaum, L.; Walker, I. D.; Garson, O. M.: Regional localization of the gene for clusterin (SP-40,40; gene symbol CLI) to human chromosome 8p12-p21. Cytogenet. Cell Genet. 61:178-179, 1992.
Dragunow, M.; Preston, K.; Dodd, J.; Young, D.; Lawlor, P.; Christie, D.: Clusterin accumulates in dying neurons following status epilepticus. Molec. Brain Res. 32: 279-290, 1995.
Duguid, J. R.; Bohmont, C. W.; Liu, N.; Tourtellotte, W. W.: Changes in brain gene expression shared by scrapie and Alzheimer disease. Proc. Nat. Acad. Sci. 86: 7260-7264, 1989.
Fink, T. M.; Zimmer, M.; Tschopp, J.; Etienne, J.; Jenne, D. E.; Lichter, P.: Human clusterin (CLI) maps to 8p21 in proximity to the lipoprotein lipase (LPL) gene. Genomics 16: 526-528, 1993.
Han, B. H.; DeMattos, R. B.; Dugan, L. L.; Kim-Han, J. S.; Brendza, R. P.; Fryer, J. D.; Kierson, M.; Cirrito, J.; Quick, K.; Harmony, J. A. K.; Aronow, B. J.; Holtzman, D. M.: Clusterin contributes to caspase-3-independent brain injury following neonatal hypoxia-ischemia. Nature Med. 7: 338-343, 2001.
Jenne, D. E.; Tschopp, J.: Clusterin: the intriguing guises of a widely expressed glycoprotein. Trends Biochem. Sci. 17: 154-159, 1992
Kamboh, M. I.; Harmony, J. A. K.; Sepehrnia, B.; Nwankwo, M.; Ferrell, R. E.: Genetic studies of human apolipoproteins. XX. Genetic polymorphism of apolipoprotein J and its impact on quantitative lipid traits in normolipidemic subjects. Am. J. Hum. Genet. 49: 1167-1173, 1991.
Kirszbaum, L.; Sharpe, J. A.; Murphy, B.; d'Apice, A. J. F.; Classon, B.; Hudson, P.; Walker, I. D.: Molecular cloning and characterization of the novel, human complement-associated protein, SP-40,40: a link between the complement and reproductive systems. EMBO J. 8: 711-718, 1989.
Murphy, B. F.; Kirszbaum, L.; Walker, I. D.; d'Apice, A. J. F.: SP-40,40, a newly identified normal human serum protein found in the SC5b-9 complex of complement and in the immune deposits in glomerulonephritis. J. Clin. Invest. 81: 1858-1864, 1988.
O'Bryan, M. K.; Baker, H. W. G.; Saunders, J. R.; Kirszbaum, L.; Walker, I. D.; Hudson, P.; Liu, D. Y.; Glew, M. D.; d'Apice, A. J. F.; Murphy, B. F.: Human seminal clusterin (SP-40,40): isolation and characterization. J. Clin. Invest. 85: 1477-1486, 1990.
Purrello, M.; Bettuzzi, S.; Di Pietro, C.; Mirabile, E.; Di Blasi, M.; Rimini, R.; Grzeschik, K.-H.; Ingletti, C.; Corti, A.; Sichel, G.: The gene for SP-40,40, human homolog of rat sulfated glycoprotein 2, rat clusterin, and rat testosterone repressed prostate message 2, maps to chromosome 8. Genomics 10: 151-156, 1991.
Slawin, K.; Sawczuk, I. S.; Olsson, C. A.; Buttyan, R.: Chromosomal assignment of the human homologue encoding SGP-2. Biochem. Biophys. Res. Commun. 172: 160-164, 1990.
Gemmill, R. M.; Bemis, L. T.; Lee, J. P.; Sozen, M. A.; Baron, A.; Zeng, C.; Erickson, P. F.; Hooper, J. E.; Drabkin, H. A.: The TRC8 hereditary kidney cancer gene suppresses growth and functions with VHL in a common pathway. Oncogene 21: 3507-3516, 2002.
Ju, Y.-T.; Chang, A. C. Y.; She, B.-R.; Tsaur, M.-L.; Hwang, H.-M.; Chao, C. C.-K.; Cohen, S. N.; Lin-Chao, S.: Gas7: a gene expressed preferentially in growth-arrested fibroblasts and terminally differentiated Purkinje neurons affects neurite formation. Proc. Nat. Acad. Sci. 95:11423-11428, 1998.
Kurtz, A.; Zimmer, A.: Interspecies fluorescence in situ hybridization further defines synteny homology between mouse chromosome 11 and human chromosome 17. Mammalian Genome 6: 379-380, 1995.
Chrast, R.; Scott, H. S.; Chen, H.; Kudoh, J.; Rossier, C.; Minoshima, S.; Wang, Y.; Shimizu, N.; Antonarakis, S. E.: Cloning of two human homologs of the Drosophila single-minded gene SIM1 on chromosome 6q and SIM2 on 21q within the Down syndrome chromosomal region. Genome Res. 7: 615-624, 1997.
Megonigal, M. D.; Cheung, N.-K. V.; Rappaport, E. F.; Nowell, P. C.; Wilson, R. B.; Jones, D. H.; Addya, K.; Leonard, D. G. B.; Kushner, B. H.; Williams, T. M.; Lange, B. J.; Felix, C. A.: Detection of leukemia-associated MLL-GAS7 translocation early during chemotherapy with DNA topoisomerase II inhibitors. Proc. Nat. Acad. Sci. 97:2814-2819, 2000.
Holder, J. L., Jr.; Butte, N. F.; Zinn, A. R.: Profound obesity associated with a balanced translocation that disrupts the SIM1 gene. Hum. Molec. Genet. 9: 101-108, 2000.
Michaud, J. L.; Boucher, F.; Melnyk, A.; Gauthier, F.; Goshu, E.; Levy, E.; Mitchell, G. A.; Himms-Hagen, J.; Fan, C.-M.: Sim1 haploin sufficiency causes hyperphagia, obesity and reduction of the paraventricular nucleus of the hypothalamus. Hum. Molec. Genet. 10: 1465-1473, 2001.
Fan, C. M.; Kuwana, E.; Bulfone, A.; Fletcher, C. F.; Copeland, N. G.; Jenkins, N. A.; Crews, S.; Martinez, S.; Puelles, L.; Rubenstein, J. L.; Tessier-Lavigne, M.: Expression patterns of two murine homologs of Drosophila single-minded suggest possible roles in embryonic patterning and in the pathogenesis of Down syndrome. Molec. Cell. Neurosci. 7:1-16, 1996.
Duke-Cohan, J. S.; Gu, J.; McLaughlin, D. F.; Xu, Y.; Freeman, G. J.; Schlossman, S. F.: Attractin (DPPT-L), a member of the CUB family of cell adhesion and guidance proteins, is secreted by activated human T lymphocytes and modulates immune cell interactions. Proc. Nat. Acad. Sci. 95: 11336-11341, 1998.
Gunn, T. M.; Miller, K. A.; He, L.; Hyman, R. W.; Davis, R. W.; Azarani, A.; Schlessman, S. F.; Duke-Cohan, J. S.; Barsh, G. S.: The mouse mahogany locus encodes a transmembrane form of human attractin. Nature 398:152-156, 1999.
He, L.; Gunn, T. M.; Bouley, D. M.; Lu, X.-Y.; Watson, S. J.; Schlossman, S. F.; Duke-Cohan, J. S.; Barsh, G. S.: A biochemical function for attractin in agouti-induced pigmentation and obesity. Nature Genet. 27:40-47, 2001.
Tang, W.; Gunn, T. M.; McLaughlin, D. F.; Barsh, G. S.; Schlossman, S. F.; Duke-Cohan, J. S.: Secreted and membrane attractin result from alternative splicing of the human ATRN gene. Proc. Nat. Acad. Sci. 97: 6025-6030, 2000.
Xiao, H.; Neuveut, C.; Tiffany, H. L.; Benkirane, M.; Rich, E. A.; Murphy, P. M.; Jeang, K.-T.: Selective CXCR4 antagonism by Tat: implications for in vivo expansion of coreceptor use by HIV-1. Proc. Nat. Acad. Sci. 97: 11466-11471, 2000.
Lin, C.-S.; Aebersold, R. H.; Kent, S. B.; Varma, M.; Leavitt, J.: Molecular cloning and characterization of plastin, a human leukocyte protein expressed in transformed human fibroblasts. Molec. Cell. Biol. 8: 4659-4668, 1988.
Lin, C.-S.; Park, T.; Chen, Z. P.; Leavitt, J.: Human plastin genes: comparative gene structure, chromosome location, and differential expression in normal and neoplastic cells. J. Biol. Chem. 268: 2781-2792, 1993.
Aggarwal, B. B.; Eessalu, T. E.; Hass, P. E.: Characterization of receptors for human tumour necrosis factor and their regulation by gamma-interferon. Nature 318: 665-667, 1985.
Evans, A. M.; Petersen, J. W.; Sekhon, G. S.; DeMars, R.: Mapping of prolactin and tumor necrosis factor-beta genes on human chromosome 6p using lymphoblastoid cell deletion mutants. Somat. Cell Molec. Genet. 15: 203-213, 1989
Gray, P. W.; Aggarwal, B. B.; Benton, C. V.; Bringman, T. S.; Henzel, W. J.; Jarrett, J. A.; Leung, D. W.; Moffat, B.; Ng, P.; Svedersky, L. P.; Palladino, M. A.; Nedwin, G. E.: Cloning and expression of cDNA for human lymphotoxin, a lymphokine with tumour necrosis activity. Nature 312:721-724, 1984.
Jongeneel, C. V.; Briant, L.; Udalova, I. A.; Sevin, A.; Nedospasov, S. A.; Cambon-Thomsen, A.: Extensive genetic polymorphism in the human tumor necrosis factor region and relation to extended HLA haplotypes. Proc. Nat. Acad. Sci. 88: 9717-9721, 1991.
Koss, K.; Satsangi, J.; Fanning, G. C.; Welsh, K. I.; Jewell, D. P.: Cytokine (TNF-alpha, LT-alpha, and IL-10) polymorphisms in inflammatory bowel diseases and normal controls: differential effects on production and allele frequencies. Genes Immun. 1: 185-190, 2000.
Leonard, W. J.; Donlon, T. A.; Lebo, R. V.; Greene, W. C.: Localization of the gene encoding the human interleukin-2 receptor on chromosome 10. Science 228: 1547-1549, 1985.
Webb, G. C.; Campbell, H. D.; Lee, J. S.; Young, I. G.: Mapping the gene for murine T-cell growth factor, II-2, to bands B-C on chromosome 3 and for the alpha chain of the IL2-receptor, II-2ra, to bands A2-A3 on chromosome 2. Cytogenet. Cell Genet. 54: 164-168, 1990.
Heinzmann, H.; Mao, X.-Q.; Akaiwa, M.; Kreomer, R. T.; Gao, P.-S.; Ohshima, K.; Umeshita, R.; Abe, Y.; Braun, S.; Yamashita, T.; Roberts, M. H.; Sugimoto, R.; and 20 others: Genetic variants of IL-13 signalling and human asthma and atopy. Hum. Molec. Genet. 9: 549-559, 2000.
Dean, F. B.; Lian, L.; O'Donnell, M.: cDNA cloning and gene mapping of human homologs for Schizosaccharomyces pombe rad17, rad1, and hus1 and cloning of homologs from mouse, Caenorhabditis elegans, and Drosophila melanogaster. Genomics 54: 424-436, 1998.
Dionne, C. A.; Kaplan, R.; Seuanez, H.; O'Brien, S. J.; Jaye, M.: Chromosome assignment by polymerase chain reaction techniques: assignment of the oncogene FGF-5 to human chromosome 4. Biotechniques 8: 190-194, 1990.
Hebert, J. M.; Rosenquist, T.; Gotz, J.; Martin, G. R.: FGF5 as a regulator of the hair growth cycle: evidence from targeted and spontaneous mutations. Cell 78: 1017-1025, 1994.
Nguyen, C.; Roux, D.; Mattei, M.-G.; de Lapeyriere, O.; Goldfarb, M.; Birnbaum, D.; Jordan, B. R.: The FGF-related oncogenes hst and int.2, and the bcl.1 locus are contained within one megabase in bandq13 of chromosome 11, while the fgf.5 oncogene maps to 4q21. Oncogene 3:703-708, 1988.
Zhan, X.; Bates, B.; Hu, X.; Goldfarb, M.: The human FGF-5 oncogene encodes a novel protein related to fibroblast growth factors. Molec. Cell. Biol. 8: 3487-3495, 1988.
Dryja, T. P.; Rapaport, J.; McGee, T. L.; Nork, T. M.; Schwartz, T. L.: Molecular etiology of low-penetrance retinoblastoma in two pedigrees. Am. J. Hum. Genet. 52: 1122-1128, 1993.
Dryja, T. P.; Rapaport, J. M.; Joyce, J. M.; Petersen, R. A.: Molecular detection of deletions involving band q14 of chromosome 13 in retinoblastomas. Proc. Nat. Acad. Sci. 83: 7391-7394, 1986.
Dryja, T. P.; Rapaport, J. M.; Weichselbaum, R.; Bruns, G. A. P.: Chromosome 13 restriction fragment length polymorphisms. Hum. Genet. 65: 320-324, 1984.
Duane, T. B.: Clinical Ophthalmology. Hagerstown: Harper and Row (pub.) 3: 1980. Pp. 13 only.
Duncan, A. M. V.; Morgan, C.; Gallie, B. L.; Phillips, R. A.; Squire, J.: Re-evaluation of the sublocalization of esterase D and its relation to the retinoblastoma locus by in situ hybridization. Cytogenet. Cell Genet. 44: 153-157, 1987.
Dunn, J. M.; Phillips, R. A.; Becker, A. J.; Gallie, B. L.: Identification of germline and somatic mutations affecting the retinoblastoma gene. Science 241:1797-1800, 1988.
Dunn, J. M.; Phillips, R. A.; Zhu, X.; Becker, A.; Gallie, B. L.: Mutations in the RB1 gene and their effects on transcription. Molec. Cell. Biol. 9: 4596-4604, 1989.
Ejima, Y.; Sasaki, M. S.; Kaneko, A.; Tanooka, H.: Types, rates, origin and expressivity of chromosome mutations involving 13q14 in retinoblastoma patients. Hum. Genet. 79: 118-123, 1988.
Ejima, Y.; Sasaki, M. S.; Kaneko, A.; Tanooka, H.; Hara, Y.; Hida, T.; Kinoshita, Y.: Possible inactivation of part of chromosome 13 due to 13qXp translocation associated with retinoblastoma. Clin. Genet. 21: 357-361, 1982.
Eldridge, R.; O'Meara, K.; Kitchin, D.: Superior intelligence in sighted retinoblastoma patients and their families. J. Med. Genet. 9:331-335, 1972.
Falls, H. F.; Neel, J. V.: Genetics of retinoblastoma. Arch. Ophthal. 46: 367-389, 1951.
Fitzgerald, P. H.; Stewart, J.; Suckling, R. D.: Retinoblastoma mutation rate in New Zealand and support for the two-hit model. Hum. Genet. 64: 128-130, 1983
Francke, U.: Retinoblastoma and chromosome 13. Cytogenet. Cell Genet. 14: 131-134, 1976.
Francois, J.: Retinoblastoma and osteogenic sarcoma. Ophthalmologica 175:185-191, 1977.
Francois, J.: Hereditary malignant tumor of the eye. Congenital Anomalies of The Eye. St. Louis: C. V. Mosby Co. (pub.) 1968. Pp. 205-246.
Francois, J.; Matton, M. T.; De Bie, S.; Tanaka, Y.; Vandenbulcke, D.: Genesis and genetics of retinoblastoma. Ophthalmologica 170:405-425, 1975.
Friend, S. H.; Bernards, R.; Rogelj, S.; Weinberg, R. A.; Rapaport, J. M.; Albert, D. M.; Dryja, T. P.: A human DNA segment with properties of the gene that predisposes to retinoblastoma and osteosarcoma. Nature 323:643-646, 1986.
Friend, S. H.; Dryja, T. P.; Weinberg, R. A.: Oncogenes and tumor-suppressing genes. New Eng. J. Med. 318: 618-622, 1988.
Friend, S. H.; Horowitz, J. M.; Gerber, M. R.; Wang, X.-F.; Bogenmann, E.; Li, F. P.; Weinberg, R. A.: Deletions of a DNA sequence in retinoblastomas and mesenchymal tumors: organization of the sequence and its encoded protein. Proc. Nat. Acad. Sci. 84: 9059-9063, 1987. Note: Correction: Proc. Nat. Acad. Sci. 85: 2234 only, 1988.
Fukushima, Y.; Kuroki, Y.; Ito, T.; Kondo, I.; Nishigaki, I.: Familial retinoblastoma (mother and son) with 13q14 deletion. Hum. Genet. 77: 104-107, 1987.
Fung, Y.-K. T.; Murphree, A. L.; T'Ang, A.; Qian, J.; Hinrichs, S. H.; Benedict, W. F.: Structural evidence for the authenticity of the human retinoblastoma gene. Science 236: 1657-1661, 1987.
Gallie, B. L.: Predictive testing for retinoblastoma comes of age. (Editorial) Am. J. Hum. Genet. 61: 279-281, 1997.
Gallie, B. L.; Ellsworth, R. M.; Abramson, D. M.; Phillips, R. A.: Retinoma: spontaneous regression of retinoblastoma or benign manifestation of the mutation? Brit. J. Cancer 45: 513-521, 1982.
Gallie, B. L.; Phillips, R. A.: Multiple manifestations of the retinoblastoma gene. Birth Defects Orig. Art. Ser. 18(6): 689-701, 1982.
Garcia-Cao, M.; Gonzalo, S.; Dean, D.; Blasco, M. A.: A role for the Rb family of proteins in controlling telomere length. Nature Genet. 15 Oct.: 2002. Note: Advance Electronic Publication.
Gey, W.: Dq-, multiple Missbildungen und Retinoblastom. Humangenetik 10:362-365, 1970
Niimi, T.; Keck-Waggoner, C. L.; Popescu, N. C.; Zhou, Y.; Levitt, R. C.; Kimura, S.: UGRP1, a uteroglobin/Clara cell secretory protein-related protein, is a novel lung-enriched downstream target gene for the T/EBP/NKX2.1 homeodomain transcription factor. Molec. Endocr. 15: 2021-2036, 2001.
Niimi, T.; Munakata, M.; Keck-Waggoner, C. L.; Popescu, N. C.; Levitt, R. C.; Hisada, M.; Kimura, S.: A polymorphism in the human UGRP1 gene promoter that regulates transcription is associated with an increased risk of asthma. Am. J. Hum. Genet. 70: 718-725, 2002.
Juwana, J.-P.; Henderikx, P.; Mischo, A.; Wadle, A.; Fadle, N.; Gerlach, K.; Arends, J. W.; Hoogenboom, H.; Pfreundschuh, M.; Renner, C.: EB/RP gene family encodes tubulin binding proteins. Int. J. Cancer 81: 275-284, 1999.
Renner, C.; Pfitzenmeier, J.-P.; Gerlach, K.; Held, G.; Ohnesorge, S.; Sahin, U.; Bauer, S.; Pfreundschuh, M.: RP1, a new member of the adenomatous polyposis coli-binding EB1-like gene family, is differently expressed in activated T cells. J. Immun. 159: 1276-1283, 1997.
Wadle, A.; Thiel, G.; Mischo, A.; Jung, V.; Pfreundschuh, M.; Renner, C.: Chromosomal localization and promoter analysis of the adenomatous polyposis coli binding protein RP1. Oncogene 20: 5920-5929, 2001.
Feng, S.-L. Y.; Guo, Y.; Factor, V. M.; Thorgeirsson, S. S.; Bell, D. W.; Testa, J. R.; Peifley, K. A.; Winkles, J. A.: The Fn14 immediate-early response gene is induced during liver regeneration and highly expressed in both human and murine hepatocellular carcinomas. Am. J. Path. 156:1253-1261, 2000.
Meighan-Mantha, R. L.; Hsu, D. K. W.; Guo, Y.; Brown, S. A. N.; Feng, S.-L. Y.; Peifley, K. A.; Alberts, G. F.; Copeland, N. G.; Gilbert, D. J.; Jenkins, N. A.; Richards, C. M.; Winkles, J. A.: The mitogen-inducible Fn14 gene encodes a type I transmembrane protein that modulates fibroblast adhesion and migration. J. Biol. Chem. 274: 33166-33176, 1999.
Eder, P. S.; Kekuda, R.; Stolc, V.; Altman, S.: Characterization of two scleroderma autoimmune antigens that copurify with human ribonuclease P. Proc. Nat. Acad. Sci. 94: 1101-1106, 1997.
Bertr and, P.; Poirier, J.; Oda, T.; Finch, C. E.; Pasinetti, G. M.: Association of apolipoprotein E genotype with brain levels of apolipoprotein E and apolipoprotein J (clusterin) in Alzheimer disease. Molec. Brain Res. 33: 174-178, 1995.
Birkenmeier, E. H.; Letts, V. A.; Frankel, W. N.; Magenheimer, B. S.; Calvet, J. P.: Sulfated glycoprotein-2 (Sgp-2) maps to mouse chromosome 14. Mammalian Genome 4: 131-132, 1993.
Danik, M.; Chabot, J.-G.; Hassan-Gonzalez, D.; Suh, M.; Quirion, R.: Localization of sulfated glycoprotein-2/clusterin mRNA in the rat brain by in situ hybridization. J. Comp. Neurol. 334: 209-227, 1993.
Wu, L.; Timmers, C.; Maiti, B.; Saavedra, H. I.; Sang, L.; Chong, G. T.; Nuckolls, F.; Giangrande, P.; Wright, F. A.; Field, S. J.; Greenberg, M. E.; Orkin, S.; Nevins, J. R.; Robinson, M. L.; Leone, G.: The E2F1-3 transcription factors are essential for cellular proliferation. Nature 414:457-462, 2001.
Bruns, G. A. P.; Regina, V. M.: Adenylate kinase-2, a mitochondrial enzyme. Biochem. Genet. 15: 477-486, 1977.
Carritt, B.; King, J.; Welch, H. M.: Gene order and localization of enzyme loci on the short arm of chromosome 1. Ann. Hum. Genet. 46:329-335, 1982.
Goss, S. J.; Harris, H.: Gene transfer by means of cell fusion. II. The mapping of 8 loci on human chromosome 1 by statistical analysis of gene assortment in somatic cell hybrids. J. Cell Sci. 25: 39-57, 1977.
Van Cong, N.; Billardon, C.; Rebourcet, R.; Kaouel, C. L.-B.; Picard, J. Y.; Weil, D.; Frezal, J.: The existence of a second adenylate kinase locus linked to PGM-1 and peptidase-C. Ann. Genet. 15: 213-218, 1972.
Pilz, A.; Woodward, K.; Povey, S.; Abbott, C.: Comparative mapping of 50 human chromosome 9 loci in the laboratory mouse. Genomics 25:139-149, 1995.
Azem, A.; Kessel, M.; Goloubinoff, P.: Characterization of a functional GroEL-14(GroES-7)-2 chaperonin hetero-oligomer. Science 265: 653-656, 1994.
Cheng, M. Y.; Hartl, F.-U.; Martin, J.; Pollock, R. A.; Kalousek, F.; Neupert, W.; Hallberg, E. M.; Hallberg, R. L.; Horwich, A. L.: Mitochondrial heat-shock protein hsp60 is essential for assembly of proteins imported into yeast mitochondria. Nature 337: 620-625, 1989.
Ellis, R. J.: The molecular chaperone concept. Semin. Cell Biol. 1:1-9, 1990.
Fontaine, B.; Davoine, C.-S.; Durr, A.; Paternotte, C.; Feki, I.; Weissenbach, J.; Hazan, J.; Brice, A.: A new locus for autosomal dominant pure spastic paraplegia, on chromosome 2q24-q34. Am. J. Hum. Genet. 66: 702-707, 2000.
Rothman, J. E.: Polypeptide chain binding proteins: catalysts of protein folding and related processes in cells. Cell 59: 591-601, 1989.
Saibil, H.; Dong, Z.; Wood, S.; auf der Mauer, A.: Binding of chaperonins. Nature 353: 25-26, 1991.
Schmidt, M.; Rutkat, K.; Rachel, R.; Pfeifer, G.; Jaenicke, R.; Viitanen, P.; Lorimer, G.; Buchner, J.: Symmetric complexes of GroEchaperonins as part of the functional cycle. Science 265: 656-659, 1994.
Venner, T. J.; Singh, B.; Gupta, R. S.: Nucleotide sequences and novel structural features of human and Chinese hamster hsp60 (chaperonin) gene families. DNA Cell Biol. 9: 545-552, 1990.
Jones, M. E. E.; Thorburn, A. W.; Britt, K. L.; Hewitt, K. N.; Wreford, N. G.; Proietto, J.; Oz, O. K.; Leury, B. J.; Robertson, K. M.; Yao, S.; Simpson, E. R.: Aromatase-deficient (ArKO) mice have a phenotype of increased adiposity. Proc. Nat. Acad. Sci. 97: 12735-12740, 2000.
Weiher, H.; Noda, T.; Gray, D. A.; Sharpe, A. H.; Jaenisch, R.: Transgenic mouse model of kidney disease: insertional inactivation of ubiquitously expressed gene leads to nephrotic syndrome. Cell 62:425-434, 1990.
Kawagishi, J.; Kumabe, T.; Yoshimoto, T.; Yamamoto, T.: Structure, organization, and transcription units of the human alpha-platelet-derived growth factor receptor gene, PDGFRA. Genomics 30: 224-232, 1995.
Allore, R.; O'Hanlon, D.; Price, R.; Neilson, K.; Willard, H. F.; Cox, D. R.; Marks, A.; Dunn, R. J.: Gene encoding the beta-subunit of S100 protein is on chromosome 21: implications for Down syndrome. Science 239:1311-1313, 1988.
Allore, R. J.; Friend, W. C.; O'Hanlon, D.; Neilson, K. M.; Baumal, R.; Dunn, R. J.; Marks, A.: Cloning and expression of the human S100-betagene. J. Biol. Chem. 265: 15537-15543, 1990.
Duncan, A. M. V.; Higgins, J.; Dunn, R. J.; Allore, R.; Marks, A.: Refined sublocalization of the human gene encoding the beta subunit of the S100 protein (S100B) and confirmation of a subtle t(9;21) translocation using in situ hybridization. Cytogenet. Cell Genet. 50: 234-235, 1989.
Reeves, R. H.; Yao, J.; Crowley, M. R.; Buck, S.; Zhang, X.; Yarowsky, P.; Gearhart, J. D.; Hilt, D. C.: Astrocytosis and axonal proliferation in the hippocampus of S100b transgenic mice. Proc. Nat. Acad. Sci. 91:5359-5363, 1994.
Kurlan, R.; Behr, J.; Medved, L.; Shoulson, I.; Pauls, D.; Kidd, J. R.; Kidd, K. K.: Familial Tourette's syndrome: report of a large pedigree and potential for linkage analysis. Neurology 36: 772-776, 1986.
Jarrous, N.; Eder, P. S.; Guerrier-Takada, C.; Hoog, C.; Altman, S.: Autoantigenic properties of some protein subunits of catalytically active complexes of human ribonuclease P. RNA 4: 407-417, 1998.
Fujimoto, K.; Shen, M.; Noshiro, M.; Matsubara, K.; Shingu, S.; Honda, K.; Yoshida, E.; Suardita, K.; Matsuda, Y.; Kato, Y.: Molecular cloning and characterization of DEC2, a new member of basic helix-loop-helix proteins. Biochem. Biophys. Res. Commun. 280: 164-171, 2001.
Garriga-Canut, M.; Roopra, A.; Buckley, N. J.: The basic helix-loop-helix protein, SHARP-1, represses transcription by a histone deacetylase-dependent and histone deacetylase-independent mechanism. J. Biol. Chem. 276:14821-14828, 2001.
Gu, H.; Saito, K.; Klaman, L. D.; Shen, J.; Fleming, T.; Wang, Y.-P.; Pratt, J. C.; Lin, G.; Lim, B.; Kinet, J.-P.; Neel, B. G.: Essential role for Gab2 in the allergic response. Nature 412: 186-190, 2001.
Albrecht, U.; Sun, Z. S.; Eichele, G.; Lee, C. C.: A differential response to two putative mammalian circadian regulators, mper1 and mper2, to light. Cell 91: 1055-1064, 1997.
Nagase, T.; Ishikawa, K.; Nakajima, D.; Ohira, M.; Seki, N.; Miyajima, N.; Tanaka, A.; Kotani, H.; Nomura, N.; Ohara, O.: Prediction of the coding sequences of unidentified human genes. VII. The complete sequences of 100 new cDNA clones from brain which can code for large proteins in vitro. DNA Res. 4: 141-150, 1997.
Prueitt, R. L.; Ross, J. L.; Zinn, A. R.: Physical mapping of-nine Xq translocation breakpoints and identification of XPNPEP2 as a premature ovarian failure candidate gene. Cytogenet. Cell Genet. 89:44-50, 2000.
Sprinkle, T. J.; Stone, A. A.; Venema, R. C.; Denslow, N. D.; Caldwell, C.; Ryan, J. W.: Assignment of the membrane-bound human aminopeptidase P gene (XPNPEP2) to chromosome Xq25. Genomics 50: 114-116, 1998.
Venema, R. C.; Ju, H.; Zou, R.; Venema, V. J.; Ryan, J. W.: Cloning and tissue distribution of human membrane-bound aminopeptidase P. Biochim. Biophys. Acta 1354: 45-48, 1997.
Ueki, N.; Oda, T.; Kondo, M.; Yano, K.; Noguchi, T.; Muramatsu, M.: Selection system for genes encoding nuclear-targeted proteins. Nat. Biotech. 16: 1338-1342, 1998.
Ueki, N.; Seki, N.; Yano, K.; Masuho, Y.; Saito, T.; Muramatsu, M.: Isolation and characterization of a novel human gene (HFB30) which encodes a protein with a RING finger motif. Biochim. Biophys. Acta 232-236, 1999.
Salbaum, J. M.: Genomic structure and chromosomal localization of the mouse gene Punc. Mammalian Genome 10: 107-111, 1999.
Yang, W.; Li, C.; Mansour, S. L.: Impaired motor coordination in mice that lack punc. Molec. Cell. Biol. 21: 6031-6043, 2001.
Fukuta, M.; Inazawa, J.; Torii, T.; Tsuzuki, K.; Shimada, E.; Habuchi, O.: Molecular cloning and characterization of human keratan sulfate gal-6-sulfotransferase. J. Biol. Chem. 272: 32321-32328, 1997.
Iida, A.; Saito, S.; Sekine, A.; Mishima, C.; Kitamura, Y.; Kondo, K.; Harigae, S.; Osawa, S.; Nakamura, Y.: Catalog of 77 single-nucleotide polymorphisms (SNPs) in the carbohydrate sulfotransferase 1 (CHST1) and carbohydrate sulfotransferase 3 (CHST3) genes. J. Hum. Genet. 47:14-19, 2002.
Mazany, K. D.; Peng, T.; Watson, C. E.; Tabas, I.; Williams, K. J.: Human chondroitin 6-sulfotransferase: cloning, gene structure, and chromosomal localization. Biochim. Biophys. Acta 1407: 92-97, 1998.
Ayres, J. A.; Shum, L.; Akarsu, A. N.; Dashner, R.; Takahashi, K.; Ikura, T.; Slavkin, H. C.; Nuckolls, G. H.: DACH: genomic characterization, evaluation as a candidate for post axial polydactyly type A2, and developmental expression pattern of the mouse homologue. Genomics 77: 18-26, 2001.
Davis, R. J.; Shen, W.; Sandler, Y. I.; Amoui, M.; Purcell, P.; Maas, R.; Ou, C.-N.; Vogel, H.; Beaudet, A. L.; Mardon, G.: Dach1 mutant mice bear no gross abnormalities in eye, limb, and brain development and exhibit postnatal lethality. Molec. Cell. Biol. 21: 1484-1490, 2001.
Hammond, K. L.; Hanson, I. M.; Brown, A. G.; Lettice, L. A.; Hill, R. E.: Mammalian and Drosophila dachshund genes are related to the Ski proto-oncogene and are expressed in eye and limb. Mech. Dev. 74:121-131, 1998.
Hammond, K. L.; Lettice, L. A.; Hill, R. E.; Lee, M.; Boyle, S.; Hanson, I. M.: Human (DACH) and mouse (Dach) homologues of Drosophila dachshund map to chromosomes 13q22 and 14E3, respectively. Genomics 55:252-253, 1999.
Li, X.; Perissi, V.; Liu, F.; Rose, D. W.; Rosenfeld, M. G.: Tissue-specific regulation of retinal and pituitary precursor cell proliferation. Science 297:1180-1183, 2002.
Blanco, G.; Irving, N. G.; Brown, S. D. M.; Miller, C. C. J.; McLoughlin, D. M.: Mapping of the human and murine X11-like genes (APBA2 and Apba2), the murine Fe65 gene (Apbbl), and the human Fe65-like gene (APBB2): genes encoding phosphotyrosine-binding domain proteins that-interact with the Alzheimer's disease amyloid precursor protein. Mammalian Genome 9: 473-475, 1998.
Loh, N. Y.; Ambrose, H. J.; Guay-Woodford, L. M.; Das-Gupta, S.; Nawrotzki, R. A.; Blake, D. J.; Davies, K. E.: Genomic organization and refined mapping of the mouse beta-dystrobrevin gene. Mammalian Genome 9: 857-862, 1998.
Peters, M. F.; O'Brien, K. F.; Sadoulet-Puccio, H. M.; Kunkel, L. M.; Adams, M. E.; Froehner, S. C.: Beta-dystrobrevin, a new member of the dystrophin family: identification, cloning, and protein associations. J. Biol. Chem. 272: 50:-31561-31569, 1997.
Cavailles, V.; Dauvois, S.; Horset, L. F.; Lopez, G.; Hoare, S.; Kushner, P. J.; Parker, M. G.: Nuclear factor RIP140 modulates transcriptional activation by the estrogen receptor. EMBO J. 14: 3741-3751, 1995.
Katsanis, N.; Ives, J. H.; Groet, J.; Nizetic, D.; Fisher, E. M. C.: Localisation of receptor interacting protein 140 (RIP140) within 100 kb of D21S13 on 21q11, a gene-poor region of the human genome. Hum. Genet. 102: 221-223, 1998.
Richelda, R.; Ronchetti, D.; Baldini, L.; Cro, L.; Viggiano, L.; Marzella, R.; Rocchi, M.; Otsuki, T.; Lombardi, L.; Maiolo, A. T.; Neri, A.: A novel chromosomal translocation t(4;14)(p16.3;q32) in multiple myeloma involves the fibroblast growth-factor receptor 3 gene. Blood 90: 4062-4070, 1997.
Baxendale, S.; MacDonald, M. E.; Mott, R.; Francis, F.; Lin, C.; Kirby, S. F.; James, M.; Zehetner, G.; Hummerich, H.; Valdes, J.; Collins, F. S.; Deaven, L. J.; Gusella, J. F.; Lehrach, H.; Bates, G. P.: A cosmid contig and high resolution restriction map of the 2 megabase region containing the Huntington's disease gene. Nature Genet. 4: 181-186, 1993.
Wright, T. J.; Ricke, D. O.; Denison, K.; Abmayr, S.; Cotter, P. D.; Hirschhorn, K.; Keinanen, M.; McDonald-McGinn, D.; Somer, M.; Spinner, N.; Yang-Feng, T.; Zackai, E.; Altherr, M. R.: A transcript map of the newly defined 165 kb Wolf-Hirschhorn syndrome critical region. Hum. Molec. Genet. 6: 317-324, 1997.
Borden, L. A.; Smith, K. E.; Gustafson, E. L.; Branchek, T. A.; Weinshank, R. L.: Cloning and expression of a betaine/GABA transporter from human brain. J. Neurochem. 64: 977-984, 1995.
Rasola, A.; Galietta, L. J. V.; Barone, V.; Romeo, G.; Bagnasco, S.: Molecular cloning and functional characterization of a GABA/betaine transporter from human kidney. FEBS Lett. 373: 229-233, 1995.
Yamauchi, A.; Uchida, S.; Kwon, H. M.; Preston, A. S.; Robey, R. B.; Garcia-Perez, A.; Burg, M. B.; Handler, J. S.: Cloning of a Na(+) and Cl(−)-dependent betaine transporter that is regulated by hypertonicity. J. Biol. Chem. 267: 649-652, 1992.
Eisses, J. F.; Kaplan, J. H.: Molecular characterization of hCTR1, the human copper uptake protein. J. Biol. Chem. 277: 29162-29171, 2002.
Klomp, A. E. M.; Tops, B. B. J.; van den Berg, I. E. T.; Berger, R.; Klomp, L. W. J.: Biochemical characterization and subcellular localization of human copper transporter 1 (hCTR1). Biochem. J. 364:497-505, 2002.
Kuo, Y.-M.; Zhou, B.; Cosco, D.; Gitschier, J.: The copper transporter CTR1 provides an essential function in mammalian embryonic development. Proc. Nat. Acad. Sci. 98: 6836-6841, 2001.
Lee, J.; Pena, M. M. O.; Nose, Y.; Thiele, D. J.: Biochemical characterization of the human copper transporter Ctrl. J. Biol. Chem. 277:4380-4387, 2002.
Lee, J.; Prohaska, J. R.; Thiele, D. J.: Essential role for mammalian copper transporter Ctrl in copper homeostasis and embryonic development. Proc. Nat. Acad. Sci. 98: 6842-6847, 2001.
Moller, L. B.; Petersen, C.; Lund, C.; Horn, N.: Characterization of the hCTR1 gene: genomic organization, functional expression, and identification of a highly homologous processed gene. Gene 257:13-22, 2000.
Zhou, B.; Gitschier, J.: hCTR1: a human gene for copper uptake identified by complementation in yeast. Proc. Nat. Acad. Sci. 94:7481-7486, 1997.
Amlal, H.; Burnham, C. E.; Soleimani, M.: Characterization of Na(+)/HCO(3−) cotransporter isoform NBC-3. Am. J. Physiol. 276:F903-F913, 1999.
Burnham, C. E.; Wang, Z.; Soleimani, M.: Personal Communication. Cincinnati, Oh. Jun. 1, 2000.
Choi, I.; Aalkjaer, C.; Boulpaep, E. L.; Boron, W. F.: An electroneutral sodium/bicarbonate cotransporter NBCn1 and associated sodium channel. Nature 405:571-575, 2000.
Ishibashi, K.; Sasaki, S.; Marumo, F.: Molecular cloning of anew sodium bicarbonate cotransporter cDNA from human retina. Biochem. Biophys. Res. Commun. 246: 535-538, 1998
Pushkin, A.; Abuladze, N.; Lee, I.; Newman, D.; Hwang, J.; Kurtz, I.: Mapping of the human NBC3 (SLC4A7) gene to chromosome 3p22. Genomics 57:321-322, 1999. Note: Correction: Genomics 58: 216 and 321-322, 1999.
Pushkin, A.; Abuladze, N.; Lee, I.; Newman, D.; Hwang, J.; Kurtz, I.: Cloning, tissue distribution, genomic organization, and functional characterization of NBC3, a new member of the sodium bicarbonate cotransporter family. J. Biol. Chem. 274: 16569-16575, 1999.
Alimova-Kost, M. V.; Imreh, S.; Buchman, V. L.; Ninkina, N. N.: Assignment of phosphotriesterase-related gene (PTER) to human chromosome band 10p12 by in situ hybridization. Cytogenet. Cell Genet. 83:16-17, 1998.
Davies, J. A.; Buchman, V. L.; Krylova, O.; Ninkina, N. N.: Molecular cloning and expression pattern of rpr-1, a resiniferatoxin-binding, phosphotriesterase-related protein, expressed in rat kidney tubules. FEBS Lett. 410: 378-382, 1997.
Pillutla, R. C.; Shimamoto, A.; Furuichi, Y.; Shatkin, A. J.: Human mRNA capping enzyme (RNGTT) and cap methyltransferase (RNMT) map to 6q16 and 18p11.22-p11.23, respectively. Genomics 54: 351-353, 1998
Ishikawa, K.; Nagase, T.; Nakajima, D.; Seki, N.; Ohira, M.; Miyajima, N.; Tanaka, A.; Kotani, H.; Nomura, N.; Ohara, O.: Prediction of the coding sequences of unidentified human genes. VIII. 78 new cDNA clones from brain which code for large proteins in vitro. DNA Res. 4:307-313, 1997.
Pillutla, R. C.; Yue, Z.; Maldonado, E.; Shatkin, A. J.: Recombinant human mRNA cap methyltransferase binds capping enzyme/RNA polymerasello complexes. J. Biol. Chem. 273: 21443-21446, 1998.
Tsukamoto, T.; Shibagaki, Y.; Niikura, Y.; Mizumoto, K.: Cloning and characterization of three human cDNAs encoding mRNA (guanine-7)-methyltransferase, an mRNA cap methylase. Biochem. Biophys. Res. Commun. 251: 27-34, 1998.
McLoughlin, D. M.; Miller, C. C. J.: The intracellular cytoplasmic domain of the Alzheimer's disease amyloid precursor protein interacts with phosphotyrosine-binding domain proteins in the yeast two-hybrid system. FEBS Lett. 397: 197-200, 1996.
Guenette, S. Y.; Chen, J.; Jondro, P. D.; Tanzi, R. E.: Association of a novel human FE65-like protein with the cytoplasmic domain of the beta-amyloid precursor protein. Proc. Nat. Acad. Sci. 93: 10832-10837, 1996.
Howard, L.; Nelson, K. K.; Maciewicz, R. A.; Blobel, C. P.: Interaction of the metalloprotease disintegrins MDC9 and MDC15 with two SH3 domain-containing proteins, endophilin I and SH3PX1. J. Biol. Chem. 274: 31693-31699, 1999.
Galliano, M.-F.; Huet, C.; Frygelius, J.; Polgren, A.; Wewer, U. M.; Engvall, E.: Binding of ADAM12, a marker of skeletal muscle regeneration, to the muscle-specific actin-binding protein, alpha-actinin-2, is required for myoblast fusion. J. Biol. Chem. 275: 13933-13939, 2000.
Gilpin, B. J.; Loechel, F.; Mattei, M.-G.; Engvall, E.; Albrechtsen, R.; Wewer, U. M.: A novel, secreted form of human ADAM 12 (meltrinalpha) provokes myogenesis in vivo. J. Biol. Chem. 273: 157-166, 1998.
Yagami-Hiromasa, T.; Sato, T.; Kurisaki, T.; Kamijo, K.; Nabeshima, Y.; Fujisawa-Sehara, A.: A metalloprotease-disintegrin participating in myoblast fusion. Nature 377: 652-656, 1995.
Cheng, G.; Ye, Z.-S.; Baltimore, D.: Binding of Bruton's tyrosine kinase to Fyn, Lyn, or Hck through a Src homology 3 domain-mediated interaction. Proc. Nat. Acad. Sci. 91: 8152-8155, 1994
Gibson, S.; Leung, B.; Squire, J. A.; Hill, M.; Arima, N.; Goss, P.; Hogg, D.; Mills, G. B.: Identification, cloning, and characterization of a novel human T-cell-specific tyrosine kinase located at the hematopoietin complex on chromosome 5q. Blood 82: 1561-1572, 1993.
Janis, E. M.; Siliciano, J. D.; Isaac, D. D.; Griffin, C. A.; Hawkins, A. L.; Kozak, C. A.; Desiderio, S.: Mapping of the gene for the tyrosine kinase Itk to a region of conserved synteny between mouse chromosome 11 and human chromosome 5q. Genomics 23: 269-271, 1994.
Schaeffer, E. M.; Debnath, J.; Yap, G.; McVicar, D.; Liao, X. C.; Littman, D. R.; Sher, A.; Varmus, H. E.; Lenardo, M. J.; Schwartzberg, P. L.: Requirement for Tec kinases Rlk and Itk in T cell receptor signaling and immunity. Science 284: 638-641, 1999.
Woods, M. L.; Kivens, W. J.; Adelsman, M. A.; Qiu, Y.; August, A.; Shimizu, Y.: A novel function for the Tec family tyrosine kinase Itk in activation of beta-1 integrins by the T-cell receptor. EMBO J. 20: 1232-1244, 2001.
Bisbal, C.; Martin and, C.; Silhol, M.; Lebleu, B.; Salehzada, T.: Cloning and characterization of a RNase L inhibitor: a new component of the interferon-regulated 2-5A pathway. J. Biol. Chem. 270: 13308-13317, 1995
Tong, Q.; Xing, S.; Jhiang, S. M.: Leucine zipper-mediated dimerization is essential for the PTC1 oncogenic activity. J. Biol. Chem. 272:9043-9047, 1997.
Orstavik, S.; Natarajan, V.; Tasken, K.; Jahnsen, T.; Sandberg, M.: Characterization of the human gene encoding the type I-alpha and type I-beta cGMP-dependent protein kinase (PRKG1). Genomics 42:311-318, 1997.
Orstavik, S.; Sandberg, M.; Berube, D.; Natarajan, V.; Simard, J.; Walter, U.; Gagne, R.; Hansson, V.; Jahnsen, T.: Localization of the human gene for type I cyclic GMP-dependent protein kinase to chromosome 10. Cytogenet. Cell Genet. 59: 270-273, 1992.
Osborne, K. A; Robichon, A.; Burgess, E.; Butland, S.; Shaw, R. A.; Coulthard, A.; Pereira, H. S.; Greenspan, R. J.; Sokolowski, M. B.: Natural behavior polymorphism due to a cGMP-dependent protein kinase of Drosophila. Science 277: 834-836, 1997.
Pfeifer, A.; Klatt, P.; Massberg, S.; Ny, L.; Sausbier, M.; Hirneill, C.; Wang, G.-X.; Korth, M.; Aszodi, A.; Andersson, K.-E.; Krombach, F.; Mayerhofer, A.; Ruth, P.; Fassler, R.; Hofmann, F.: Defective smooth muscle regulation in cGMP kinase 1-deficient mice. EMBO J. 17:3045-3051, 1998.
Sandberg, M.; Natarajan, V.; Ronander, I.; Kalderon, D.; Walter, U.; Lohmann, S. M.; Jahnsen, T.: Molecular cloning and predicted full-length amino acid sequence of the type I beta isozyme of cGMP-dependent protein kinase from human placenta: tissue distribution and developmental changes in rat. FEBS Lett. 255: 321-329, 1989.
Tamura, N.; Itoh, H.; Ogawa, Y.; Nakagawa, O.; Harada, M.; Chun, T.-H.; Suga, S.; Yoshimasa, T.; Nakao, K.: cDNA cloning and gene expression of human type I-alpha cGMP-dependent protein kinase. Hypertension 27:552-557, 1996.
Oyen, O.; Myklebust, F.; Scott, J. D.; Hansson, V.; Jahnsen, T.: Human testis cDNA for the regulatory subunit RII alpha of cAMP-dependent protein kinase encodes an alternate amino-terminal region. FEBS Lett. 246:57-64, 1989.
Tasken, K.; Naylor, S. L.; Solberg, R.; Jahnsen, T.: Mapping of the gene encoding the regulatory subunit RII-alpha of cAMP-dependent protein kinase (locus PRKAR2A) to human chromosome region 3p21.3-p21.2. Genomics 50:378-381, 1998.
Solberg, R.; Sistonen, P.; Traskelin, A.-L.; Berube, D.; Simard, J.; Krajci, P.; Jahnsen, T.; de la Chapelle, A.: Mapping of the regulatory subunits R1-beta and RII-beta of cAMP-dependent protein kinase genes on human chromosome 7. Genomics 14: 63-69, 1992.
Cummings, D. E.; Brandon, E. P.; Planas, J. V.; Motamed, K.; Idzerda, R. L.; McKnight, G. S.: Genetically lean mice result from targeted disruption of the RII-beta subunit of protein kinase A. Nature 382::622-626, 1996.
Scambler, P.; Oyen, O.; Wainwright, B.; Farrall, M.; Law, H.-Y.; Estivill, X.; Sandberg, M.; Williamson, R.; Jahnsen, T.: Exclusion of catalytic and regulatory subunits of cAMP-dependent protein kinase as candidate genes for the defect causing cystic fibrosis. Am. J. Hum. Genet. 41: 925-932, 1987.
Wainwright, B.; Lench, N.; Davies, K.; Scambler, P.; Kruyer, H.; Williamson, R.; Jahnsen, T.; Farrall, M.: A human regulatory subunit of type II cAMP-dependent protein kinase localized by its linkage relationship to several cloned chromosome 7q markers. Cytogenet. Cell Genet. 45: 237-239, 1987.
Akiyama, K.; Yokota, K.; Kagawa, S.; Shimbara, N.; Tamura, T.; Akioka, H.; Nothwang, H. G.; Noda, C.; Tanaka, K.; Ichihara, A.: cDNA cloning and interferon gamma down-regulation of proteasomal subunits X and Y. Science 265: 1231-1234, 1994.
Driscoll, J.; Brown, M. G.; Finley, D.; Monaco, J. J.: MHC-linked LMP gene products specifically alter peptidase activities of the proteasome. Nature 365:262-264, 1993.
Gaczynska, M.; Rock, K. L.; Goldberg, A. L.: Gamma-interferon and expression of MHC genes regulate peptide hydrolysis by proteasomes. Nature 365:264-267, 1993.
Glynne, R.; Powis, S. H.; Beck, S.; Kelly, A.; Kerr, L. A.; Trowsdale, J.: A proteasome-related gene between the two ABC transporter loci in the class II region of the human MHC. Nature 353: 357-360, 1991.
Lin, K.; Thomas, J. T.; McBride, O. W.; Luyten, F. P.: Assignment of a new TGF-beta super family member, human cartilage-derived morphogenetic protein-1, to chromosome 20q11.2. Genomics 34: 150-151, 1996.
To be, T.; Minoshima, S.; Yamase, S.; Choi, N.-H.; Tomita, M.; Shimizu, N.: Assignment of a human serum glyco protein SP-40,40 gene (CLI) to chromosome 8. Cytogenet. Cell Genet. 57: 193-195, 1991.
Wong, P.; Pineault, J.; Lakins, J.; Taillefer, D.; Leger, J.; Wang, C.; Tenniswood, M.: Genomic organization and expression of the rat TRPM-2 (clusterin) gene, a gene implicated in apoptosis. J. Biol. Chem. 268: 5021-5031, 1993.
Wong, P.; Taillefer, D.; Lakins, J.; Pineault, J.; Chader, G.; Tenniswood, M.: Molecular characterization of human TRPM-2/clusterin, a gene associated with sperm maturation, apoptosis and neurodegeneration. Europ. J. Biochem. 221: 917-925, 1994.
Phillips, S. A.; Barr, V. A.; Haft, D. H.; Taylor, S. I.; Haft, C. R.: Identification and characterization of SNX15, a novel sorting nexin involved in protein trafficking. J. Biol. Chem. 276: 5074-5084, 2001.
Roberts, W. M.; Look, A. T.; Ruossel, M. F.; Sherr, C. J.: Tandem linkage of human CSF-1 receptor (c-fms) and PDGF receptor genes. Cell 55:655-661, 1988.
Smith, E. A.; Seldin, M. F.; Martinez, L.; Watson, M. L.; Ghosh Choudhury, G.; Lalley, P. A.; Pierce, J.; Aaronson, S.; Barker, J.; Naylor, S. L.; Sakaguchi, A. Y.: Mouse platelet-derived growth factor receptor alpha gene is deleted in W-19H and patch mutations on chromosomes. Proc. Nat. Acad. Sci. 88: 4811-4815, 1991.
Stenman, G.; Eriksson, A.; Claesson-Welsh, L.: Human PDGFA receptor gene maps to the same region on chromosome 4 as the KIT oncogene. Genes Chromosomes Cancer 1: 155-158, 1989.
Stephenson, D. A.; Mercola, M.; Anderson, E.; Wang, C.; Stiles, C. D.; Bowen-Pope, D. F.; Chapman, V. M.: Platelet-derived growth factor receptor alpha-subunit gene (Pdgfra) is deleted in the mouse patch (Ph) mutation. Proc. Nat. Acad. Sci. 88: 6-10, 1991.
Xie, J.; Aszterbaum, M.; Zhang, X.; Bonifas, J. M.; Zachary, C.; Epstein, E.; McCormick, F.: A role of PDGFR-alpha in basal cell carcinoma proliferation. Proc. Nat. Acad. Sci. 98: 9255-9259, 2001.
Moog-Lutz, C.; Bouillet, P.; Regnier, C. H.; Tomasetto, C.; Mattei, M. G.; Chenard, M. P.; Anglard, P.; Rio, M. C.; Basset, P.: Comparative expression of the psorias in (S100A7) and S100C genes in breast carcinoma and co-localization to human chromosome 1q21-q22. Int. J. Cancer 63:297-303, 1995.
Tanaka, M.; Adzuma, K.; Iwami, M.; Yoshimoto, K.; Monden, Y.; Itakura, M.: Human calgizzarin: one colorectal cancer-related gene selected by a large scale random cDNA sequencing and northern blot analysis. Cancer Lett. 89: 195-200, 1995.
Todoroki, H.; Kobayashi, R.; Watanabe, M.; Minami, H.; Hidaka, H.: Purification, characterization, and partial sequence analysis of a newly identified EF-hand type 13-kDa Ca(2+)-binding protein from smooth muscle and non-muscle tissues. J. Biol. Chem. 266: 18668-18673, 1991
Watanabe, M.; Ando, Y.; Todoroki, H.; Minami, H.; Hidaka, H.: Molecular cloning and sequencing of a cDNA clone encoding a new calcium binding protein, named calgizzarin, from rabbit lung. Biochem. Biophys. Res. Commun. 181: 644-649, 1991.
Sullivan, J. L.; Byron, K. S.; Brewster, F. E.; Baker, S. M.; Ochs, H. D.: X-linked lymphoproliferative syndrome: natural history of the immunodeficiency. J. Clin. Invest. 71: 1765-1778, 1983.
Sullivan, J. L.; Byron, K. S.; Brewster, F. E.; Purtilo, D. T.: Deficient natural killer cell activity in X-linked lymphoproliferative syndrome. Science 210: 543-545, 1980.
Sumazaki, R.; Kanegane, H.; Osaki, M.; Fukushima, T.; Tsuchida, M.; Matsukura, H.; Shinozaki, K.; Kimura, H.; Matsui, A.; Miyawaki, T.: SH2D1A mutations in Japanese males with severe Epstein-Barr virus-associated illnesses. Blood 98: 1268-1270, 2001.
Sumegi, J.; Gross, T. G.; Seemayer, T. A.: The molecular genetics of X-linked lymphoproliferative (Duncan's) disease. Cancer J. Sci. Am. 5: 57-62, 1999.
Sumegi, J.; Huang, D.; Lanyi, A.; Davis, J. D.; Seemayer, T. A.; Maeda, A.; Klein, G.; Seri, M.; Wakiguchi, H.; Purtilo, D. T.; Gross, T. G.: Correlation of mutations of the SH2D1A gene and Epstein-Barrvirus infection with clinical phenotype and outcome in X-linked lymphoproliferative disease. Blood 96: 3118-3125, 2000.
Xiang, X.; Benson, K. F.; Chada, K.: Mini-mouse: disruption of the pygmy locus in a transgenic insertional mutant. Science 247:967-969, 1990.
Scriver, C. R.: Vitamin B6 deficiency and dependency in man. Am. J. Dis. Child. 113: 109-114, 1967.
Scriver, C. R.; Hutchison, J. H.: The vitamin B6 deficiency syndrome in human infancy: biochemical and clinical observations. Pediatrics 31:240-250, 1963.
Uchida, T.; Fujimori, F.; Tradler, T.; Fischer, G.; Rahfeld, J.-U.: Identification and characterization of a 14 kDa human protein as a novel parvulin-like peptidyl prolyl cis/trans isomerase. FEBS Lett. 446:278-282, 1999.
Shearman, L. P.; Sriram, S.; Weaver, D. R.; Maywood, E. S.; Chaves, I.; Zheng, B.; Kume, K.; Lee, C. C.; van der Horst, G. T. J.; Hastings, M. H.; Reppert, S. M.: Interacting molecular loops in the mammalian circadian clock. Science 288: 1013-1019, 2000.
Charlier, C.; Coppieters, W.; Farnir, F.; Grobet, L.; Leroy, P. L.; Michaux, C.; Mni, M.; Schwers, A.; Vanmanshoven, P.; Hanset, R.; Georges, M.: The mh gene causing double-muscling in cattle maps to bovine chromosome 2. Mammalian Genome 6: 788-792, 1995.
Ferrell, R. E.; Conte, V.; Lawrence, E. C.; Roth, S. M.; Hagberg, J. M.; Hurley, B. F.: Frequent sequence variation in the human myostatin (GDF8) gene as a marker for analysis of muscle-related phenotypes. Genomics 62:203-207, 1999.
Gonzalez-Cadavid, N. F.; Taylor, W. E.; Yarasheski, K.; Sinha-Hikim, I.; Ma, K.; Ezzat, S.; Shen, R.; Lalani, R.; Asa, S.; Mamita, M.; Nair, G.; Arver, S.; Bhasin, S.: Organization of the human myostatin gene and expression in healthy men and HIV-infected men with muscle wasting. Proc. Nat. Acad. Sci. 95: 14938-14943, 1998.
Grobet, L.; Martin, L. J. R.; Poncelet, D.; Pirottin, D.; Brouwers, B.; Riquet, J.; Schoeberlein, A.; Dunner, S.; Menissier, F.; Massabanda, J.; Fries, R.; Hanset, R.; Georges, M.: A deletion in the bovine myostatin gene causes the double-muscled phenotype in cattle. Nature-Genet. 17: 71-74, 1997.
Chikuba, K.; Yubisui, T.; Shirabe, K.; Takeshita, M.: Cloning and nucleotide sequence of a cDNA of the human erythrocyte NADPH-flavin reductase. Biochem. Biophys. Res. Commun. 198: 1170-1176, 1994
Saito, F.; Yamaguchi, T.; Komuro, A.; To be, T.; Ikeuchi, T.; Tomita, M.; Nakajima, H.: Mapping of the newly identified biliverdin-IX beta-reductase gene (BLVRB) to human chromosome 19q13.13-q13.2 by fluorescence in situ hybridization. Cytogenet. Cell Genet. 71: 179-181, 1995.
Yamaguchi, T.; Komuro, A.; Nakano, Y.; Tomita, M.; Nakajima, H.: Complete amino acid sequence of biliverdin-IX beta reductase from human liver. Biochem. Biophys. Res. Commun. 197: 1518-1523, 1993.
Kosaki, K.; Bassi, M. T.; Kosaki, R.; Lewin, M.; Belmont, J.; Schauer, G.; Casey, B.: Characterization and mutation analysis of human LEFTYA and LEFTY B, homologues of murine genes implicated in left-right axis development. Am. J. Hum. Genet. 64: 712-721, 1999.
Kothapalli, R.; Buyuksal, I.; Wu, S.-Q.; Chegini, N.; Tabibzadeh, S.: Detection of ebaf, a novel human gene of the transforming growth factor beta super family: association of gene expression with endometrial bleeding. J. Clin. Invest. 99: 2342-2350, 1997.
Meno, C.; Itoh, Y.; Saijoh, Y.; Matsuda, Y.; Tashiro, K.; Kuhara, S.; Hamada, H.: Two closely-related left-right asymmetrically expressed genes, lefty-1 and lefty-2: their distinct expression domains, chromosomal linkage and direct neuralizing activity in Xenopus embryos. Genes Cells 2: 513-524, 1997.
Meno, C.; Saijoh, Y.; Fujii, H.; Ikeda, M.; Yokoyama, T.; Yokoyama, M.; Toyoda, Y.; Hamada, H.: Left-right asymmetric expression of the TGF-beta-family member lefty in mouse embryos. Nature 381: 151-155, 1996.
Meno, C.; Shimono, A.; Saijoh, Y.; Yashiro, K.; Mochida, K.; Ohishi, S.; Noji, S.; Kondoh, H.; Hamada, H.: Lefty-1 is required for left-right determination as a regulator of lefty-2 and nodal. Cell 94: 287-297, 1998.
Tabibzadeh, S.; Mason, J. M.; Shea, W.; Cai, Y.; Murray, M. J.; Lessey, B.: Dysregulated expression of ebaf, a novel molecular defect in the endometria of patients with infertility. J. Clin. Endocr. Metab. 85: 2526-2536, 2000.
Kamitani, T.; Chang, H.-M.; Rollins, C.; Waneck, G. L.; Yeh, E. T. H.: Correction of the class A defect in glycosylphosphatidylinositol anchor biosynthesis in Ltk-cells by human cDNA clone. J. Biol. Chem. 268:20733-20736, 1993.
Cirullo, R. E.; Arredondo-Vega, F. X.; Smith, M.; Wasmuth, J. J.: Isolation and characterization of interspecific heat-resistant hybrids between a temperature-sensitive Chinese hamster cell asparaginyl-tRNA synthetase mutant and normal human leukocytes: assignment of human asnS gene to chromosome 18. Somat. Cell Genet. 9: 215-233, 1983.
Shows, T. B.: Personal Communication. Buffalo, N.Y. Jan. 11, 1983.
Lin, S.-C.; Lin, C. R.; Gukovsky, I.; Lusis, A. J.; Sawchenko, P. E.; Rosenfeld, M. G.: Molecular basis of the little mouse phenotype and implications for cell type-specific growth. Nature 364: 208-213, 1993.
Itoh, H.; Tomita, M.; Uchino, H.; Kobayashi, T.; Kataoka, H.; Sekiya, R.; Nawa, Y.: cDNA cloning of rat pS2 peptide and expression of trefoil peptides in acetic acid-induced colitis. Biochem. J. 318: 939-944, 1996.
Seib, T.; Blin, N.; Hilgert, K.; Seifert, M.; Theisinger, B.; Engel, M.; Dooley, S.; Zang, K.-D.; Welter, C.: The three human trefoil genes TFF1, TFF2, and TFF3 are located within a region of 55 kb on chromosome 21q22.3. Genomics 40: 200-202, 1997.
Hruban, R. H.; Goggins, M.; Parsons, J.; Kern, S. E.: Clin. Cancer Res. 6: 2969-2972, 2000.
Hruban, R. H.; van Mansfield, A. D. M.; Offerhaus, G. J. A.; van Weering, D. H. J.; Allison, D. C.; Goodman, S. N., Kensler, T. W.; Bose, K. K.; Cameron, J. L.; Bos, J. L.: Am. J. Path. 143: 545-554, 1993.
Hruban, R. H.; Wilentz, R. E.; Kern, S. E.: Am. J. Pathol. 156:1821-1825, 2000.
Fazioli, F.; Minichiello, L.; Matoska, V.; Castagnino, P.; Miki, T.; Wong, W. T.; Di Fiore, P. P.: Eps8, a substrate for the epidermal growth factor receptor kinase, enhances EGF-dependent mitogenic signals. EMBO J. 12: 3799-3808, 1993.
Kumar, S.; Tomooka, Y.; Noda, M.: Identification of a set of genes with developmentally down-regulated expression in the mouse brain. Biochem. Biophys. Res. Commun. 185: 1155-1161, 1992.
Braybrooke, J. P.; Spink, K. G.; Thacker, J.; Hickson, I. D.: The RAD51 family member, RAD51L3, is a DNA-stimulated ATPase that forms a complex with XRCC2. J. Biol. Chem. 275: 29100-29106, 2000.
Johnson, R. D.; Liu, N.; Jasin, M.: Mammalian XRCC2 promotes the repair of DNA double-strand breaks by homologous recombination. Nature 401:397-399, 1999.
Jones, N. J.; Cox, R.; Thacker, J.: Isolation and cross-sensitivity of x-ray-sensitive mutants of V79-4 hamster cells. Mutat. Res. 183:279-286, 1987.
Jones, N. J.; Zhao, Y.; Siciliano, M. J.; Thompson, L. H.: Assignment of the XRCC2 human DNA repair gene to chromosome 7q36 by complementation analysis. Genomics 26: 619-622, 1995.
Kurumizaka, H.; Ikawa, S.; Nakada, M.; Enomoto, R.; Kagawa, W.; Kinebuchi, T.; Yamazoe, M.; Yokoyama, S.; Shibata, T.: Homologous pairing and ring and filament structure formation activities of the human Xrcc2-Rad51D complex. J. Biol. Chem. 277: 14315-14320, 2002.
Liu, N.; Schild, D.; Thelen, M. P.; Thompson, L. H.: Involvement of Rad51C in two distinct protein complexes of Rad51 paralogs in human cells. Nucleic Acids Res. 30: 1009-1015, 2002.
Masson, J.-Y.; Tarsounas, M. C.; Stasiak, A. Z.; Stasiak, A.; Shah, R.; McIlwraith, M. J.; Benson, F. E.; West, S. C.: Identification and purification of two distinct complexes containing the five RAD51 paralogs. Genes Dev. 15: 3296-3307, 2001.
Tambini, C. E.; George, A. M.; Rommens, J. M.; Tsui, L.-C.; Scherer, S. W.; Thacker, J.: The XRCC2 DNA repair gene: identification of a positional candidate. Genomics 41: 84-92, 1997.
Thacker, J.; Tambini, C. E.; Simpson, P. J.; Tsui, L.-C.; Scherer, S. W.: Localization to chromosome 7q36.1 of the human XRCC2 gene, determining sensitivity to DNA-damaging agents. Hum. Molec. Genet. 4:113-120, 1995
Nakagawara, A.; Liu, X.-G.; Ikegaki, N.; White, P. S.; Yamashiro, D. J.; Nycum, L. M.; Biegel, J. A.; Brodeur, G. M.: Cloning and chromosomal localization of the human TRKB tyrosine kinase receptor gene (NTRK2). Genomics 25:538-546, 1995.
Rico, B.; Xu, B.; Reichardt, L. F.: TrkB receptor signaling is-required for establishment of GABAergic synapses in the cerebellum. Nature Neurosci. 5: 225-233, 2002.
Slaugenhaupt, S. A.; Blumenfeld, A.; Liebert, C. B.; Mull, J.; Lucente, D. E.; Monahan, M.; Breakefield, X. O.; Maayan, C.; Parada, L.; Axelrod, F. B.; Gusella, J. F.: The human gene for neurotrophic tyrosine kinase receptor type 2 (NTRK2) is located on chromosome 9 but is not the familial dysautonomia gene. Genomics 25: 730-732, 1995.
Soppet, D.; Escandon, E.; Maragos, J.; Middlemas, D. S.; Reid, S. W.; Blair, J.; Burton, L. E.; Stanton, B. R.; Kaplan, D. R.; Hunter, T.; Nikolics, K.; Parada, L. F.: The neurotrophic factors brain-derived neurotrophic factor and neurotrophin-3 are ligands for the trkB tyrosine kinase receptor. Cell 65: 895-903, 1991.
Squinto, S. P.; Stitt, S. N.; Aldrich, T. H.; Davis, S.; Bianco, S. M.; Radziejewski, C.; Glass, D. J.; Masiakowski, P.; Furth, M. E.; Valenzuela, D. M.; DiStefano, P. S.; Yancopoulos, G. D.: trkB encodes a functional receptor for brain-derived neurotrophic factor and neurotrophin-3 but not nerve growth factor. Cell 65: 885-893, 1991.
Burks, D. J.; Font de Mora, J.; Schubert, M.; Withers, D. J.; Myers, M. G.; Towery, H. H.; Altamuro, S. L.; Flint, C. L.; White, M. F.: IRS-2 pathways integrate female reproduction and energy homeostasis. Nature 407:377-382, 2000.
Fritsche, A.; Madaus, A.; Renn, W.; Tschritter, O.; Teigeler, A.; Weisser, M.; Maerker, E.; Machicao, F.; Haring, H.; Stumvoll, M.: The prevalent Gly1057Asp polymorphism in the insulin receptor substrate-2 gene is not associated with impaired insulin secretion. J. Clin. Endocr. Metab. 86: 4822-4825, 2001.
Kubota, N.; To be, K.; Terauchi, Y.; Eto, K.; Yamauchi, T.; Suzuki, R.; Tsubamoto, Y.; Komeda, K.; Nakano, R.; Miki, H.; Satoh, S.; Sekihara, H.; Sciacchitano, S.; Lesniak, M.; Aizawa, S.; Nagai, R.; Kimura, S.; Akanuma, Y.; Taylor, S. I.; Kadowaki, T.: Disruption of insulin receptor substrate 2 causes type 2 diabetes because of liver insulin resistance and lack of compensatory beta-cell hyperplasia. Diabetes 49::-1880-1889, 2000.
Sun, X. J.; Wang, L.-M.; Zhang, Y.; Yenush, L.; Myers, M. G., Jr.; Glasheen, E.; Lane, W. S.; Pierce, J. H.; White, M. F.: Role of IRS-2 in insulin and cytokine signalling. Nature 377: 173-177, 1995.
Withers, D. J.; Burks, D. J.; Towery, H. H.; Altamuro, S. L.; Flint, C. L.; White, M. F.: Irs-2 coordinates Igf-1 receptor-mediated beta-cell development and peripheral insulin signalling. Nature Genet. 23:32-40, 1999.
Withers, D. J.; Gutierrez, J. S.; Towery, H.; Burks, D. J.; Ren, J.-M.; Previs, S.; Zhang, Y.; Bernal, D.; Pons, S.; Shulman, G. I.; Bonner-Weir, S.; White, M. F.: Disruption of IRS-2 causes type 2 diabetes in mice. Nature 391: 900-902, 1998.
Wong, W. T.; Carlomagno, F.; Druck, T.; Barletta, C.; Croce, C. M.; Huebner, K.; Kraus, M. H.; Di Fiore, P. P.: Evolutionary conservation of the EPS8 gene and its mapping to human chromosome 12q23-q24. Oncogene 9:3057-3061, 1994.
Peters, M. A.; Ostrander, E. A.: Prostate cancer: more than two to tango. Nature Genet. 27: 134-135, 2001.
Chen, S. L.; Dowhan, D. H.; Hosking, B. M.; Muscat, G. E. O.: The steroid receptor coactivator, GRIP-1, is necessary for MEF-2C-dependent gene expression and skeletal muscle differentiation. Genes Dev. 14:1209-1228, 2000.
Yoshida, M. C.; Sasaki, M.; Mise, K.; Semba, K.; Nishizawa, M.; Yamamoto, T.; Toyoshima, K.: Regional mapping of the human proto-oncogene c-yes-1 to chromosome 18 at band q21.3. Jpn. J. Cancer Res. 76:559-562, 1985.
Goossens, M.; Brauner, R.; Czernichow, P.; Duquesnoy, P.; Rappaport, R.: Isolated growth hormone (GH) deficiency type 1A associated with a double deletion in the human GH gene cluster. J. Clin. Endocr. Metab. 62: 712-716, 1986.
Cool, D. R.; Normant, E.; Shen, F.; Chen, H.-C.; Pannell, L.; Zhang, Y.; Loh, Y. P.: Carboxypeptidase E is a regulated secretory pathway sorting receptor: genetic obliteration leads to endocrine disorders in Cpe(fat) mice. Cell 88: 73-83, 1997.
Nicoloso, M.; Caizergues-Ferrer, M.; Michot, B.; Azum, M.-C.; Bachellerie, J.-P.: U20, a novel small nucleolar RNA, is encoded in an intron of the nucleolin gene in mammals. Molec. Cell. Biol. 14: 5766-5776, 1994.
Srivastava, M.; Fleming, P. J.; Pollard, H. B.; Burns, A. L.: Cloning and sequencing of the human nucleolin cDNA. FEBS Lett. 250:99-105, 1989.
Srivastava, M.; McBride, O. W.; Fleming, P. J.; Pollard, H. B.; Burns, A. L.: Genomic organization and chromosomal localization of the human nucleolin gene. J. Biol. Chem. 265: 14922-14931, 1990
Campbell, G. R.; Zimmerman, K.; Blank, R. D.; Alt, F. W.; D'Eustachio, P.: Chromosomal location of N-myc and L-myc genes in the mouse. Oncogene Res. 4: 47-54, 1989.
Bahou, W. F.; Nierman, W. C.; Durkin, A. S.; Potter, C. L.; Demetrick, D. J.: Chromosomal assignment of the human thrombin receptor gene: localization to region q13 of chromosome 5. Blood 82: 1532-1537, 1993.
Coughlin, S. R.; Vu, T.-K. H.; Hung, D. T.; Wheaton, V. I.: Characterization of a functional thrombin receptor: issues and opportunities. J. Clin. Invest. 89: 351-355, 1992.
Griffin, C. T.; Srinivasan, Y.; Zheng, Y.-W.; Huang, W.; Coughlin, S. R.: A role for thrombin receptor signaling in endothelial cells during embryonic development. Science 293: 1666-1670, 2001.
Poirier, C.; O'Brien, E. P.; Bueno Brunialti, A. L.; Chambard, J.-C.; Swank, R. T.; Guenet, J.-L.: The gene encoding the thrombin receptor (Cf2r) maps to mouse chromosome 13. Mammalian Genome 7:322, 1996.
Schmidt, V. A.; Nierman, W. C.; Feldblyum, T. V.; Maglott, D. R.; Bahou, W. F.: The human thrombin receptor and proteinase activated receptor-2 genes are tightly linked on chromosome 5q13. Brit. J. Haemat. 97: 523-529, 1997.
Schmidt, V. A.; Vitale, E.; Bahou, W. F.: Genomic cloning and characterization of the human thrombin receptor gene: structural similarity to the proteinase activated receptor-2 gene. J. Biol. Chem. 271:9307-9312, 1996.
Vu, T.-K. H.; Hung, D. T.; Wheaton, V. I.; Coughlin, S. R.: Molecular cloning of a functional thrombin receptor reveals a novel proteolytic mechanism of receptor activation. Cell 64: 1057-1068, 1991.
Bravo, R.: Synthesis of the nuclear protein cyclin (PCNA) and its relationship with DNA replication. Exp. Cell Res. 163: 287-293, 1986.
Hasan, S.; Hassa, P. O.; Imhof, R.; Hottiger, M. O.: Transcription coactivator p300 binds PCNA and may have a role in DNA repair synthesis. Nature 410:387-391, 2001.
Hoege, C.; Pfander, B.; Moldovan, G.-L.; Pyrowolakis, G.; Jentsch, S.: RAD6-dependent DNA repair is linked to modification of PCNA by ubiquitin and SUMO. Nature 419: 135-141, 2002.
Ku, D.-H.; Travali, S.; Calabretta, B.; Huebner, K.; Baserga, R.: Human gene for proliferating cell nuclear antigen has pseudo genes and localizes to chromosome 20. Somat. Cell Molec. Genet. 15: 297-307, 1989.
Mann, M. J.; Gibbons, G. H.; Kernoff, R. S.; Diet, F. P.; Tsao, P. S.; Cooke, J. P.; Kaneda, Y.; Dzau, V. J.: Genetic engineering of vein grafts resistant to atherosclerosis. Proc. Nat. Acad. Sci. 92:4502-4506, 1995.
Rao, V. V. N. G.; Schnittger, S.; Hansmann, I.: Chromosomal localization of the human proliferating cell nuclear antigen (PCNA) gene to or close to 20p12 by in situ hybridization. Cytogenet. Cell Genet. 56:169-170, 1991.
Suzuka, I.; Daidoji, H.; Matsuoka, M.; Kadowaki, K.; Takasaki, Y.; Nakane, P. K.; Moriuchi, T.: Gene for proliferating-cell nuclear antigen (DNA polymerase delta auxiliary protein) is present in both mammalian and higher plant genomes. Proc. Nat. Acad. Sci. 86: 3189-3193, 1989.
Taniguchi, Y.; Katsumata, Y.; Koido, S.; Suemizu, H.; Yoshimura, S.; Moriuchi, T.; Okumura, K.; Kagotani, K.; Taguchi, H.; Imanishi, T.; Gojobori, T.; Inoko, H.: Cloning, sequencing, and chromosomal localization of two tandemly arranged human pseudo genes for the proliferating cell nuclear antigen (PCNA). Mammalian Genome 7: 906-908, 1996.
Travali, S.; Ku, D.-H.; Rizzo, M. G.; Ottavio, L.; Baserga, R.; Calabretta, B.: Structure of the human gene for the proliferating cell nuclear antigen. J. Biol. Chem. 264: 7466-7472, 1989.
Webb, G.; Parsons, P.; Chenevix-Trench, G.: Localization of the gene for human proliferating nuclear antigen/cyclin by in situ hybridization. Hum. Genet. 86: 84-86, 1990.
Fonatsch, C.; Duchrow, M.; Rieder, H.; Schluter, C.; Gerdes, J.: Assignment of the human Ki-67 gene (MKI67) to 10q25-qter. Genomics 11:476-477, 1991.
Schluter, C.; Duchrow, M.; Wohlenberg, C.; Becker, M. H. G; Key, G.; Flad, H.-D.; Gerdes, J.: The cell proliferation-associated antigen of antibody Ki-67: a very large, ubiquitous nuclear protein with numerous repeated elements, representing a new kind of cell cycle-maintaining proteins. J. Cell. Biol. 123: 513-522, 1993.
Schonk, D. M.; Kuijpers, H. J. H.; van Drunen, E.; van Dalen, C. H.; Geurts van Kessel, A. H. M.; Verheijen, R.; Ramaekers, F. C. S.: Assignment of the gene(s) involved in the expression of the proliferation-related Ki-67 antigen to human chromosome 10. Hum. Genet. 83: 297-299, 1989.
Traut, W.; Scholzen, T.; Winking, H.; Kubbutat, M. H. G.; Gerdes, J.: Assignment of the murine Ki-67 gene (Mki67) to chromosome band 7F3-F5 by in situ hybridization. Cytogenet. Cell Genet. 83: 12-13, 1998.
Fukata, M.; Watanabe, T.; Noritake, J.; Nakagawa, M.; Yamaga, M.; Kuroda, S.; Matsuura, Y.; Iwamatsu, A.; Perez, F.; Kaibuchi, K.: Rac1 and Cdc42 capture microtubules through IQGAP1 and CLIP-170. Cell 109:873-885, 2002.
Brown, M. A.; Nicolai, H.; Xu, C.-F.; Griffiths, B. L.; Jones, K. A.; Solomon, E.; Hosking, L.; Trowsdale, J.; Black, D. M.; McFarlane, R.: Regulation of BRCA1. (Letter) Nature 372: 733 only, 1994.
Brown, M. A.; Xu, C.-F.; Nicolai, H.; Griffiths, B.; Chambers, J. A.; Black, D.; Solomon, E.: The 5-prime end of the BRCA1 genelies within a duplicated region of human chromosome 17q21. Oncogene 12:2507-2513, 1996.
Campbell, I. G.; Nicolai, H. M.; Foulkes, W. D.; Senger, G.; Stamp, G. W.; Allan, G.; Boyer, C.; Jones, K.; Bast, R. C., Jr.; Solomon, E.; Trowsdale, J.; Black, D. M.: A novel gene encoding a B-box protein within the BRCA1 region at 17q21.1. Hum. Molec. Genet. 3: 589-594, 1994.
Kawashima, K.; Shikama, H.; Imoto, K.; Izawa, M.; Naruke, T.; Okabayashi, K.; Nishimura, S.: Close correlation between restriction fragment length polymorphism of the L-MYC gene and metastasis of human lung cancer to the lymph nodes and other organs. Proc. Nat. Acad. Sci. 85:2353-2356, 1988.
Kaye, F.; Battey, J.; Nau, M.; Brooks, B.; Seifter, E.; De Greve, J.; Birrer, M.; Sausville, E.; Minna, J.: Structure and expression of the human L-myc gene reveal a complex pattern of alternative mRNA processing. Molec. Cell. Biol. 8: 186-195, 1988.
McBride, O. W.; Kirsch, I.; Hollis, G.; Nau, M.; Battey, J.; Minna, J.: Human L-myc (MYCL) proto-oncogene is on chromosome 1p32. (Abstract) Cytogenet. Cell Genet. 40: 694 only, 1985.
Nau, M. M.; Brooks, B. J.; Battey, J.; Sausville, E.; Gazdar, A. F.; Kirsch, I. R.; McBride, O. W.; Bertness, V.; Hollis, G. F.; Minna, J. D.: L-myc, a new myc-related gene amplified and expressed in humansmall cell lung cancer. Nature 318: 69-73, 1985.
Rouleau, G. A.; Bazanowski, A.; Gusella, J. F.; Haines, J. L.: A genetic map of chromosome 1: comparison of different data sets and linkage programs. Genomics 7: 313-318, 1990.
Speleman, F.; Van Camp, G.; Van Roy, N.: Reassignment of MYCL1 to human chromosome 1p34.3 by fluorescence in situ hybridization. Cytogenet. Cell Genet. 72: 189-190, 1996.
Van Roy, N.; Cheng, N. C.; Laureys, G.; Opdenakker, G.; Versteeg, R.; Speleman, F.: Molecular cytogenetic analysis of 1; 17 translocations in neuroblastoma. Europ. J. Cancer 31A: 530-535, 1995
Akiyama, T.; Sudo, C.; Ogawara, H.; Toyoshima, K.; Yamamoto, T.: The product of the human c-erbB-2 gene: a 185-kilodalton glyco protein with tyrosine kinase activity. Science 232: 1644-1646, 1986.
Ameyaw, M.-M.; Tayeb, M.; Thornton, N.; Folayan, G.; Tariq, M.; Mobarek, A.; Evans, D. A. P.; Ofori-Adjei, D.; McLeod, H. L.: Ethnic variation in the HER-2 codon 655 genetic polymorphism previously associated with breast cancer. J. Hum. Genet. 47: 172-175, 2002.
Chen, S.-H.; Anderson, J. E.; Giblett, E. R.: Human red cell 2,3-diphosphoglyceratemutase and monophosphoglycerate mutase: genetic evidence for two separate loci. Am. J. Hum. Genet. 29: 405-407, 1977.
Pol, S.; Bousquet-Lernercier, B.; Pave-Preux, M.; Pawlak, A.; Nalpas, B.; Berthelot, P.; Hanoune, J.; Barouki, R.: Nucleotide sequence and tissue distribution of the human mitochondrial aspartate aminotransferase mRNA. Biochem. Biophys. Res. Commun. 157: 1309-1315, 1988.
Becker, D. M.; Fikes, J. D.; Guarente, L.: A cDNA encoding a human CCAAT-binding protein cloned by functional complementation in yeast. Proc. Nat. Acad. Sci. 88: 1968-1972, 1991.
Parmentier, M.; Passage, E.; Vassart, G.; Mattei, M.-G.: The human calbindin D28k (CALB1) and calretinin (CALB2) genes are located at 8q21.3-q22.1 and 16q22-q23, respectively, suggesting a common duplication with the carbonic anhydrase isozyme loci. Cytogenet. Cell Genet. 57:41-43, 1991.
Parmentier, M.; Szpirer, J.; Levan, G.; Vassart, G.: The human genes for calbindin 27 and 29 kDa proteins are located on chromosomes 8 and 16, respectively. Cytogenet. Cell Genet. 52: 85-87, 1989.
Schiffmann, S. N.; Cheron, G.; Lohof, A.; d'Alcantara, P.; Meyer, M.; Parmentier, M.; Schurmans, S.: Impaired motor coordination and Purkinje cell excitability in mice lacking calretinin. Proc. Nat. Acad. Sci. 96: 5257-5262, 1999.
Bibel, M.; Barde, Y.-A.: Neurotrophins: key regulators of cell fate and cell shape in the vertebrate nervous system. Genes Dev. 14:2919-2937, 2000.
Bothwell, M.: p75(NTR): a receptor after all. Science 272: 506-507, 1996.
Carter, B. D.; Lewin, G. R.: Neurotrophins live or let die: does p75(NTR) decide? Neuron 18: 187-190, 1997.
Jackson, R. S.; Creemers, J. W. M.; Ohagi, S.; Raffin-Sanson, M.-L.; Sanders, L.; Montague, C. T.; Hutton, J. C.; O'Rahilly, S.: Obesity and impaired prohormone processing associated with mutations in the human prohormone convertase 1 gene. Nature Genet. 16: 303-306, 1997.
Naggert, J. K.; Fricker, L. D.; Varlamov, O.; Nishina, P. M.; Rouille, Y.; Steiner, D. F.; Carroll, R. J.; Paigen, B. J.; Leiter, E. H.: Hyperproinsulinaemia in obese fat/fat mice associated with a carboxypeptidase E mutation which reduces enzyme activity. Nature Genet. 10: 135-142, 1995.
O'Rahilly, S.; Gray, H.; Humphreys, P. J.; Krook, A.; Polonsky, K. S.; White, A.; Gibson, S.; Taylor, K.; Carr, C.: Brief report: impaired processing of prohormones associated with abnormalities of glucose homeostasis and adrenal function. New Eng. J. Med. 333:1386-1390, 1995.
Ohagi, S.; Sakaguchi, H.; Sanke, T.; Tatsuta, H.; Hanabusa, T.; Nanjo, K.: Human prohormone convertase 3 gene: exon-intron organization and molecular scanning for mutations in Japanese subjects with NIDDM. Diabetes 45:897-901, 1996.
Furuta, M.; Carroll, R.; Martin, S.; Swift, H.; Ravazzola, M.; Orci, L.; Steiner, D.: Incomplete processing of proinsulin to insulin accompanied by elevation of Des-31,32 proinsulin intermediates in islets of mice lacking active PC2. J. Biol. Chem. 273: 3431-3437, 1998.
Furuta, M.; Yano, H.; Zhou, A.; Rouille, Y.; Holst, J.; Caroll, R.; Ravazzola, M.; Orci, L.; Furuta, H.; Steiner, D.: Defective prohormone processing and altered pancreatic islet morphology in mice lacking active SPC2. Proc. Nat. Acad. Sci. 94: 6646-6651, 1997.
Furuta, M.; Zhou, A.; Webb, G.; Carroll, R.; Ravazzola, M.; Orci, L.; Steiner, D. F.: Severe defect in proglucagon processing in islet A-cells of prohormone convertase 2 null mice. J. Biol. Chem. 276:27197-27202, 2001.
Maglott, D. R.; Feldblyum, T. V.; Durkin, A. S.; Nierman, W. C.: Radiation hybrid mapping of SNAP, PCSK2, and THBD (human chromosome 20p). Mammalian Genome 7: 400-401, 1996.
Ohagi, S.; LaMendola, J.; LeBeau, M. M.; Espinosa, R., III; Takeda, J.; Smeekens, S. P.; Chan, S. J.; Steiner, D. F.: Identification and analysis of the gene encoding human PC2, a prohormone convertase expressed in neuroendocrine tissues. Proc. Nat. Acad. Sci. 89: 4977-4981, 1992.
Taylor, N. A.; Shennan, K. I. J.; Cutler, D. F.; Docherty, K.: Mutations within the propeptide, the primary cleavage site or the catalytic site, or deletion of C-terminal sequences, prevents secretion of proPC2 from transfected COS-7 cells. Biochem. J. 321: 367-373, 1997.
Gabreels, B. A. T. F.; Swaab, D. F.; de Kleijn, D. P. V.; Seidah, N. G.; Van de Loo, J.-W.; Van de Ven, W. J. M.; Martens, G. J. M. and van Leeuwen, F. W.: Attenuation of the polypeptide 7B2, prohormone convertase PC2, and vasopressin in the hypothalamus of some Prader-Willi-patients: indications for a processing defect. J. Clin. Endocr. Metab. 83:591-599, 1998.
Arbiser, J. L.; Morton, C. C.; Bruns, G. A. P.; Majzoub, J. A.: Human corticotropin releasing hormone gene is located on the long arm of chromosome 8. Cytogenet. Cell Genet. 47: 113-116, 1988.
Behan, D. P.; Heinrichs, S. C.; Troncoso, J. C.; Liu, X.-J.; Kawas, C. H.; Ling, N.; De Souza, E. B.: Displacement of corticotropin releasing factor from its binding protein as a possible treatment for Alzheimer's disease. Nature 378: 284-287, 1995.
Cheng, Y.-H.; Nicholson, R. C.; King, B.; Chan, E.-C.; Fitter, J. T.; Smith, R.: Corticotropin-releasing hormone gene expression in primary placental cells is modulated by cyclic adenosine 3-prime, 5-prime-monophosphate. J. Clin. Endocr. Metab. 85: 1239-1244, 2000.
Habib, K. E.; Weld, K. P.; Rice, K. C.; Pushkas, J.; Champoux, M.; Listwak, S.; Webster, E. L.; Atkinson, A. J.; Schulkin, J.; Contoreggi, C.; Chrousos, G. P.; McCann, S. M.; Suomi, S. J.; Higley, J. D.; Gold, P. W.: Oral administration of a corticotropin-releasing hormone receptor antagonist significantly attenuates behavioral, neuroendocrine, and autonomic responses to stress in primates. Proc. Nat. Acad. Sci. 97:6079-6084, 2000.
Inder, W. J.; Prickett, T. C. R.; Ellis, M. J.; Hull, L.; Reid, R.; Benny, P. S.; Livesey, J. H.; Donald, R. A.: The utility of plasma CRH as a predictor of preterm delivery. J. Clin. Endocr. Metab. 86:5706-5710, 2001.
Kellogg, J.; Luty, J. A.; Thompson, R.; Luo, X. Y.; Magenis, R. E.; Litt, M.: Corticotropin releasing hormone (CRH) maps to human chromosome 8 and identifies a Taql RFLP. Cytogenet. Cell Genet. 51:1022, 1989.
Knapp, L. T.; Keegan, C. E.; Seasholtz, A. F.; Camper, S. A.: Corticotropin-releasing hormone (Crh) maps to mouse chromosome 3. Mammalian Genome 4: 615-617, 1993.
Kyllo, J. H.; Collins, M. M.; Vetter, K. L.; Cuttler, L.; Rosenfield, R. L.; Donohoue, P. A.: Linkage of congenital isolated adrenocorticotropichormone deficiency to the corticotropin releasing hormone locus using simple sequence repeat polymorphisms. Am. J. Med. Genet. 62: 262-267, 1996.
Majzoub, J. A.: Personal Communication. Boston, Mass. Mar. 3, 1995.
Mandel, H.; Berant, M.; Gotfried, E.; Hochberg, Z.: Autosomal recessive hypothalamic corticotropin deficiency: a new entity and its metabolic consequences. (Abstract) Am. J. Hum. Genet. 47 (suppl.): A66, 1990.
McLean, M.; Bisits, A.; Davies, J.; Woods, R.; Lowry, P.; Smith, R.: A placental clock controlling the length of human pregnancy. Nature Med. 1: 460-463, 1995.
Muglia, L.; Jacobson, L.; Dikkes, P.; Majzoub, J. A.: Corticotropin-releasing hormone deficiency reveals major fetal but not adult glucocorticoid need. Nature 373: 427-432, 1995.
Robinson, B. G.; Emanuel, R. L.; Frim, D. M.; Majzoub, J. A.: Glucocorticoid stimulates expression of corticotropin-releasing hormone gene in human placenta. Proc. Nat. Acad. Sci. 85: 5244-5248, 1988.
Scatena, C. D.; Adler, S.: Trans-acting factors dictate the species-specific placental expression of corticotropin-releasing factor genes in choriocarcinoma cell lines. Endocrinology 137: 3000-3008, 1996.
Scatena, C. D.; Adler, S.: Characterization of a human-specific regulator of placental corticotropin-releasing hormone. Molec. Endocr. 12:1228-1240, 1998
Shibahara, S.; Morimoto, Y.; Furutani, Y.; Notake, M.; Takahashi, H.; Shimizu, S.; Horikawa, S.; Numa, S.: Isolation and sequence analysis of the human corticotropin-releasing factor precursor gene. EMBO J. 2: 775-779, 1983.
Stratakis, C. A.; Chrousos, G. P.: Neuroendocrinology and pathophysiology of the stress system. Ann. N.Y. Acad. Sci. 771: 1-18, 1995.
Stratakis, C. A.; Sarlis, N. J.; Berrettini, W. H.; Badner, J. A.; Chrousos, G. P.; Gershon, E. S.; Detera-Wadleigh, S. D.: Lack of linkage between the corticotropin-releasing hormone (CRH) gene and bipolar affective disorder. Molec. Psychiat. 2: 483-485, 1997.
Xu, B.; Sano, T.; Yamada, S.; Li, C. C.; Hirokawa, M.: Expression of corticotropin-releasing hormone messenger ribonucleic acid in human pituitary corticotroph adenomas associated with proliferative potential. J. Clin. Endocr. Metab. 85: 1220-1225, 2000.
Zouboulis, C. C.; Seltmann, H.; Hiroi, N.; Chen, W.; Young, M.; Oeff, M.; Scherbaum, W. A.; Orfanos, C. E.; McCann, S. M.; Bornstein, S. R.: Corticotropin-releasing hormone: an autocrine hormone that promotes lipogenesis in human sebocytes. Proc. Nat. Acad. Sci. 99:7148-7153, 2002.
Chen, R.; Lewis, K. A.; Perrin, M. H.; Vale, W. W.: Expression cloning of a human corticotropin-releasing-factor receptor. Proc. Nat. Acad. Sci. 90: 8967-8971, 1993.
Dieterich, K. D.; Gundelfinger, E. D.; Ludecke, D. K.; Lehnert, H.: Mutation and expression analysis of corticotropin-releasing factor 1 receptor in adrenocorticotropin-secreting pituitary adenomas. J. Clin. Endocr. Metab. 83: 3327-3331, 1998.
Grammatopoulos, D.; Dai, Y.; Chen, J.; Karteris, E.; Papadopoulou, N.; Easton, A. J.; Hilihouse, E. W.: Human corticotropin-releasing hormone receptor: differences in subtype expression between pregnant and nonpregnant myometria. J. Clin. Endocr. Metab. 83: 2539-2544, 1998.
Leproult, R.; Colecchia, E. F.; L'Hermite-Baleriaux, M.; Van Cauter, E.: Transition from dim to bright light in the morning induces an immediate elevation of cortisol levels. J. Clin. Endocr. Metab. 86:151-157, 2001.
Liaw, C. W.; Grigoriadis, D. E.; Lovenberg, T. W.; De Souza, E. B.; Maki, R. A.: Localization of ligand-binding domains of human corticotropin-releasing factor receptor: a chimeric receptor approach. Molec. Endocr. 11: 980-985, 1997.
Danielson, K. G.; Fazzio, A.; Cohen, I.; Cannizzaro, L. A.; Eichstetter, I.; Iozzo, R. V.: The human decorin gene: intron-exon organization, discovery of two alternatively spliced exons in the 5-prime untranslated region, and mapping of the gene to chromosome 12q23. Genomics 15:146-160, 1993.
Dyne, K. M.; Valli, M.; Forlino, A.; Mottes, M.; Kresse, H.; Cetta, G.: Deficient expression of the small proteoglycan decorin in a case of severe/lethal osteogenesis imperfecta. Am. J. Med. Genet. 63:161-166, 1996.
Ion, A.; Crosby, A. H.; Kremer, H.; Kenmochi, N.; Van Reen, M.; Fenske, C.; Van Der Burgt, I.; Brunner, H. G.; Montgomery, K.: Detailed mapping, mutation analysis, and intragenic polymorphism identification in candidate Noonan syndrome genes MYL2, DCN, EPS8, and RPL6. J. Med. Genet. 37: 884-886, 2000.
McBride, O. W.; Fisher, L. W.; Young, M. F.: Localization of PGI (biglycan, BGN) and PGII (decorin, DCN, PG-40) genes on human chromosomes Xq13-qter and 12q, respectively. Genomics 6: 219-255, 1990.
Moscatello, D. K.; Santra, M.; Mann, D. M.; McQuillan, D. J.; Wong, A. J.; Iozzo, R. V.: Decorin suppresses tumor cell growth by activating the epidermal growth factor receptor. J. Clin. Invest. 101: 406-412, 1998.
Pulkkinen, L.; Alitalo, T.; Krusius, T.; Peltonen, L.: Expression of decorin in human tissues and cell lines and defined chromosomal assignment of the gene locus (DCN). Cytogenet. Cell Genet. 60: 107-111, 1992.
Pulkkinen, L.; Kainulainen, K.; Krusius, T.; Makinen, P.; Schollin, J.; Gustavsson, K.-H.; Peltonen, L.: Deficient expression of the gene coding for decorin in a lethal form of Marfan syndrome. J. Biol. Chem. 265: 17780-17785, 1990.
Schollin, J.; Bjarke, B.; Gustavson, K.-H.: Probable homozygotic form of the Marfan syndrome in a newborn child. Acta Paediat. Scand. 77:452-456, 1988.
Scholzen, T.; Solursh, M.; Suzuki, S.; Reiter, R.; Morgan, J. L.; Buchberg, A. M.; Siracusa, L. D.; Iozzo, R. V.: The murine decorin: complete cDNA cloning, genomic organization, chromosomal assignment, and expression during organogenesis and tissue differentiation. J. Biol. Chem. 269: 28270-28281, 1994.
Vogel, K. G.; Clark, P. E.: Small proteoglycan synthesis by skin fibroblasts cultured from elderly donors and patients with defined defects in types I and III collagen metabolism. Europ. J. Cell Biol. 49:236-243, 1989.
Yoon, J.-W.; Yoon, C.-S.; Lim, H.-W.; Huang, Q. Q.; Kang, Y.; Pyun, K. H.; Hirasawa, K.; Sherwin, R. S.; Jun, H.-S.: Control of autoimmune diabetes in NOD mice by GAD expression or suppression in beta cells. Science 284: 1183-1187, 1999.
Asakura, H.; Zwain, I. H.; Yen, S. S. C.: Expression of genes encoding corticotropin-releasing factor (CRF), type 1 CRF receptor, and CRF-binding protein and localization of the gene products in the human ovary. J. Clin. Endocr. Metab. 82: 2720-2725, 1997.
Byerley, W.; Hoff, M.; Holik, J.; Caron, M. G.; Giros, B.: VNTR polymorphism for the human dopamine transporter gene (DAT1). Hum. Molec. Genet. 2: 335, 1993.
Cook, E. H., Jr.; Stein, M. A.; Krasowski, M. D.; Cox, N. J.; Olkon, D. M.; Kieffer, J. E.; Leventhal, B. L.: Association of attention-deficit disorder and the dopamine transporter gene. Am. J. Hum. Genet. 56:993-998, 1995.
Doucette-Stamm, L.; Blakely, D. J.; Tian, J.; Mockus, S.; Mao, J.: Population genetic study of the human dopamine transporter gene (DAT1). Genet. Epidemiol. 12: 303-308, 1995.
Gainetdinov, R. R.; Wetsel, W. C.; Jones, S. R.; Levin, E. D.; Jaber, M.; Caron, M. G.: Role of serotonin in the paradoxical calming effect of psychostimulants on hyperactivity. Science 283: 397-401, 1999.
Gelernter, J.; Vandenbergh, D.; Kruger, S. D.; Pauls, D. L.; Kurlan, R.; Pakstis, A. J.; Kidd, K. K.; Uhl, G.: The dopamine transporter protein gene (SLC6A3): primary linkage mapping and linkage studies in Tourette syndrome. Genomics 30: 459-463, 1995.
Gill, M.; Daly, G.; Heron, S.; Hawi, Z.; Fitgerald, M.: Confirmation of association between attention deficit hyperactivity disorder and a dopamine transporter polymorphism. Molec. Psychiat. 2: 311-313, 1997.
Giros, B.; E1 Mestikawy, S.; Godinot, N.; Zheng, K.; Han, H.; Yang-Feng, T.; Caron, M. G.: Cloning, pharmacological characterization, and chromosome assignment of the human dopamine transporter. Molec. Pharm. 42:383-390, 1992.
Giros, B.; Jaber, M.; Jones, S. R.; Wightman, R. M.; Caron, M. G.: Hyperlocomotion and indifference to cocaine and amphetamine in mice lacking the dopamine receptor. Nature 370: 606-612, 1996.
Goldman, D.: Dopamine transporter, alcoholism and other diseases. Nature Med. 1: 624-625, 1995.
Andrechek, E. R.; Hardy, W. R.; Girgis-Gabardo, A. A.; Perry, R. L. S.; Butler, R.; Graham, F. L.; Kahn, R. C.; Rudnicki, M. A.; Muller, W. J.: ErbB2 is required for muscle spindle and myoblast cell survival. Molec. Cell. Biol. 22: 4714-4722, 2002.
Bargmann, C. I.; Hung, M.-C.; Weinberg, R. A.: The NEU oncogene encodes an epidermal growth factor receptor-related protein. Nature 319:226-230, 1986.
Chan, R.; Hardy, W. R.; Laing, M. A.; Hardy, S. E.; Muller, W. J.: The catalytic activity of the ErbB-2 receptor tyrosine kinase is essential for embryonic development. Molec. Cell. Biol. 22: 1073-1078, 2002.
Coussens, L.; Yang-Feng, T. L.; Liao, Y.-C.; Chen, E.; Gray, A.; McGrath, J.; Seeburg, P. H.; Libermann, T. A.; Schlessinger, J.; Francke, U.; Levinson, A.; Ullrich, A.: Tyrosine kinase receptor with extensive homology to EGF receptor shares chromosomal location with NEU oncogene. Science 230:1132-1139, 1985.
Crone, S. A.; Zhao, Y.-Y.; Fan, L.; Gu, Y.; Minamisawa, S.; Liu, Y.; Peterson, K. L.; Chen, J.; Kahn, R.; Condorelli, G.; Ross, J., Jr.; Chien, K. R.; Lee, K.-F.: ErbB2 is essential in the prevention of dilated cardiomyopathy. Nature Med. 8: 459-465, 2002.
Dankort, D.; Maslikowski, B.; Warner, N.; Kanno, N.; Kim, H.; Wang, Z.; Moran, M. F.; Oshima, R. G.; Cardiff, R. D.; Muller, W. J.: Grb2 and Shc adapter proteins play distinct roles in Neu (ErbB-2) induced mammary tumorigenesis: implications for human breast cancer. Molec. Cell. Biol. 21: 1540-1551, 2001.
De Boer, J. G.: A new mutator phenotype in breast cancer? (Commentary) Proc. Nat. Acad. Sci. 99: 3368-3369, 2002.
De Placido, S.; Carlomagno, C.; De Laurentiis, M.; Bianco, A. R.: C-erbB2 expression predicts tamoxifen efficacy in breast cancer patients. Breast Cancer Res. Treat. 52: 55-64, 1998.
Di Fiore, P. P.; Pierce, J. H.; Kraus, M. H.; Segatto, O.; King, C. R.; Aaronson, S. A.: erbB-2 is a potent oncogene when overexpressed in NIH/3T3 cells. Science 237: 178-182, 1987.
Doherty, J. K.; Bond, C.; Jardim, A.; Adelman, J. P.; Clinton, G. M.: The HER-2/neu receptor tyrosine kinase gene encodes a secreted autoinhibitor. Proc. Nat. Acad. Sci. 96: 10869-10874, 1999.
Ehsani, A.; Low, J.; Wallace, R. B.; Wu, A. M.: Characterization of a new allele of the human ERBB2 gene by allele-specific competition hybridization. Genomics 15: 426-429, 1993.
Francke, U.: Personal Communication. New Haven, Conn. 4/1988.
Fukushige, S.-I.; Matsubara, K.-I.; Yoshida, M.; Sasaki, M.; Suzuki, T.; Semba, K.; Toyoshima, K.; Yamamoto, T.: Localization of a novel v-erbB-related gene, c-erbB-2, on human chromosome 17 and its amplification in a gastric cancer cell line. Molec. Cell. Biol. 6: 955-958, 1986.
Kaneko, Y.; Homma, C.; Maseki, N.; Sakurai, M.; Toyoshima, K.; Yamamoto, T.: Human c-erbB-2 remains on chromosome 17 in band q21 in the 15;17 translocation associated with acute promyelocytic leukemia. Jpn. J. Cancer Res. 78: 16-19, 1987.
Lin, W.; Sanchez, H. B.; Deerinck, T.; Morris, J. K.; Ellisman, M.; Lee, K. F.: Aberrant development of motor axons and neuromuscular synapses in erbB2-deficient mice. Proc. Nat. Acad. Sci. 97: 1299-1304, 2000.
Liu, S.; Liu, W.; Jakubczak, J. L.; Erexson, G. L.; Tindall, K. R.; Chan, R.; Muller, W. J.; Adhya, S.; Garges, S.; Merlino, G.: Genetic instability favoring transversions associated with ErbB2-induced mammary tumorigenesis. Proc. Nat. Acad. Sci. 99: 3770-3775, 2002.
Mehta, R. R.; McDermott, J. H.; Hieken, T. J.; Marler, K. C.; Patel, M. K.; Wild, L. D.; Das Gupta, T. K.: Plasma c-erbB2 levels in breast cancer patients: prognostic significance in predicting response to chemotherapy. J. Clin. Oncol. 16: 2409-2416, 1998
Morris, J. K.; Lin, W.; Hauser, C.; Marchuk, Y.; Getman, D.; Lee, K.-F.: Rescue of the cardiac defect in ErbB2 mutant mice reveals essential roles of ErbB2 in peripheral nervous system development. Neuron 23:273-283, 1999.
Muleris, M.; Almeida, A.; Malfoy, B.; Dutrillaux, B.: Assignment of v-erb-b2 avian erythroblastic leukemia viral oncogene homolog 2(ERBB2) to human chromosome band 17q21.1 by in situ hybridization. Cytogenet. Cell Genet. 76: 34-35, 1997.
Ozcelik, C.; Erdmann, B.; Pilz, B.; Wettschureck, N.; Britsch, S.; Hubner, N.; Chien, K. R.; Birchmeier, C.; Garratt, A. N.: Conditional mutation of the ErbB2 (HER2) receptor in cardiomyocytes leads to dilated cardiomyopathy. Proc. Nat. Acad. Sci. 99: 8880-8885, 2002.
Papewalis, J.; Nikitin, A. Y.; Rajewsky, M. F.: G to A polymorphism at amino acid codon 655 of the human erbB-2/HER2 gene. Nucleic Acids Res. 19: 5452 only, 1991.
Pegram, M. D.; Finn, R. S.; Arzoo, K.; Beryt, M.; Pietras, R. J.; Slamon, D. J.: The effect of HER-2/neu overexpression on chemotherapeutic drug sensitivity in human breast and ovarian cancer cells. Oncogene 15:537-547, 1997.
Pietras, R. J.; Pegram, M. D.; Finn, R. S.; Maneval, D. A.; Slamon, D. J.: Remission of human breast cancer xenografts on therapy with humanized monoclonal antibody to HER-2 receptor and DNA-reactive drugs. Oncogene 17:2235-2249, 1998.
Popescu, N. C.; King, C. R.; Kraus, M. H.: Localization of the human erbB-2 gene on normal and rearranged chromosomes 17 to bands q12-21.32. Genomics 4: 362-366, 1989.
Qiu, Y.; Ravi, L.; Kung, H.-J.: Requirement of ErbB2 for signalling by interleukin-6 in prostate carcinoma cells. Nature 393: 83-85, 1998.
Semba, K.; Kamata, N.; Toyoshima, K.; Yamamoto, T.: A v-erbB-related protooncogene, c-erbB-2, is distinct from the c-erbB-1/epidermal growth factor-receptor gene and is amplified in a human salivary gland adenocarcinoma. Proc. Nat. Acad. Sci. 82: 6497-6501, 1985.
Slamon, D. J.; Godolphin, W.; Jones, L. A.; Holt, J. A.; Wong, S. G.; Keith, D. E.; Levin, W. J.; Stuart, S. G.; Udove, J.; Ullrich, A.; Press, M. F.: Studies of the HER-2/neu protooncogene in human breast and ovarian cancer. Science 244: 707-712, 1989.
Slamon, D. J.; Leyland-Jones, B.; Shak, S.; Fuchs, H.; Paton, V.; Bajamonde, A.; Fleming, T.; Eiermann, W.; Wolter, J.; Pegram, M.; Baselga, J.; Norton, L.: Use of chemotherapy plus a monoclonal antibody against HER2 for metastatic breast cancer that overexpresses HER2. New Eng. J. Med. 344: 783-792, 2001.
Scita, G.; Nordstrom, J.; Carbone, R.; Tenca, P.; Giardina, G.; Gutkind, S.; Bjarnegard, M.; Betsholtz, C.; Di Fiore, P. P.: EPS8 and E3B1 transduce signals from Ras to Rac. Nature 401: 290-293, 1999.
Zuniga, A.; Haramis, A.-P. G.; McMahon, A. P.; Zeller, R.: Signal relay by BMP antagonism controls the SHH/FGF4 feedback loop in vertebrate limb buds. Nature 401: 598-602, 1999.
Heine, P. A.; Taylor, J. A.; Iwamoto, G. A.; Lubahn, D. B.; Cooke, P. S.: Increased adipose tissue in male and female estrogen receptor-alpha knockout mice. Proc. Nat. Acad. Sci. 97: 12729-12734, 2000.
Cook, P. J. L.; Hamerton, J. L.: Report of the committee on the genetic constitution of chromosome 1. Cytogenet. Cell Genet. 25:9-20, 1979.
Karasawa, M.; Zwacka, R. M.; Reuter, A.; Fink, T.; Hsieh, C. L.; Lichter, P.; Francke, U.; Weiher, H.: The human homolog of the glomerulosclerosis gene Mpv17: structure and genomic organization. Hum. Molec. Genet. 2:1829-1834, 1993
Boyes, J.; Bird, A.: DNA methylation inhibits transcription indirectly via a methyl-CpG binding protein. Cell 64: 1123-1134, 1991.
Cross, S. H.; Meehan, R. R.; Nan, X.; Bird, A.: A component of the transcriptional repressor MeCP1 shares a motif with DNA methyltransferase and HRX proteins. Nature Genet. 16: 256-259, 1997.
Hendrich, B.; Abbott, C.; McQueen, H.; Chambers, D.; Cross, S.; Bird, A.: Genomic structure and chromosomal mapping of the murine and human Mbd1, Mbd2, Mbd3, and Mbd4 genes. Mammalian Genome 10:906-912, 1999.
Beauchemin, N.; Draber, P.; Dveksler, G.; Gold, P.; Gray-Owen, S.; Grunert, F.; Hammarstrom, S.; Holmes, K. V.; Karlsson, A.; Kuroki, M.; Lin, S.-H.; Lucka, L.; and 13 others: Redefined nomenclature for members of the carcinoembryonic antigen family. Exp. Cell Res. 252:243-249, 1999.
Compton, D. A.; Szilak, I.; Cleveland, D. W.: Primary structure of NuMA, an intranuclear protein that defines a novel pathway for segregation of proteins at mitosis. J. Cell Biol. 116: 1395-1408, 1992.
Lydersen, B. K.; Pettijohn, D. E.: Human-specific nuclear protein that associates with the polar region of the mitotic apparatus: distribution in a human/hamster hybrid cell. Cell 22: 489-499, 1980.
Merdes, A.; Ramyar, K.; Vechio, J. D.; Cleveland, D. W.: A complex of NuMA and cytoplasmic dynein is essential for mitotic spindle assembly. Cell 87:447-458, 1996.
Sparks, C. A.; Bangs, P. L.; McNeil, G. P.; Lawrence, J. B.; Fey, E. G.: Assignment of the nuclear mitotic apparatus protein NuMA gene to human chromosome 1q13. Genomics 17: 222-224, 1993.
Wells, R. A.; Catzavelos, C.; Kamel-Reid, S.: Fusion of retinoic acid receptor alpha to NuMA, the nuclear mitotic apparatus protein, by a variant translocation in acute promyelocytic leukaemia. Nature Genet. 17: 109-113, 1997.
Wiese, C.; Wilde, A.; Moore, M. S.; Adam, S. A.; Merdes, A.; Zheng, Y.: Role of importin-beta in coupling Ran to downstream targets in microtubule assembly. Science 291: 653-656, 2001.
Yang, C. H.; Lambie, E. J.; Snyder, M.: NuMA: an unusually long coiled-coil related protein in the mammalian nucleus. J. Cell Biol. 116:1303-1317, 1992.
Nomura, N.; Nagase, T.; Miyajima, N.; Sazuka, T.; Tanaka, A.; Sato, S.; Seki, N.; Kawarabayasi, Y.; Ishikawa, K.; Tabata, S.: Prediction of the coding sequences of unidentified human genes. II. The coding sequences of 40 new genes (KIAA0041-KIAA0080) deduced by analysis of cDNA clones from human cell line KG-1. DNA Res. 1: 223-229, 1994.
Bezieau, S.; Devilder, M.-C.; Avet-Loiseau, H.; Mellerin, M.-P.; Puthier, D.; Pennarun, E.; Rapp, M.-J.; Harousseau, J.-L.; Moisan, J.-P.; Bataille, R.: High incidence of N and K-Ras activating mutations in multiple myeloma and primary plasma cell leukemia at diagnosis. Hum. Mutat. 18: 212-224, 2001.
Popescu, N. C.; Amsbaugh, S. C.; DiPaolo, J. A.; Tronick, S. R.; Aaronson, S. A.; Swan, D. C.: Chromosomal localization of three human ras genes by in situ molecular hybridization. Somat. Cell Molec. Genet. 11: 149-155, 1985.
Maggi, M.; Barni, T.; Fantoni, G.; Mancina, R.; Pupilli, C.; Luconi, M.; Crescioli, C.; Serio, M.; Vannelli, G. B.: Expression and biological effects of endothelin-1 in human gonadotropin-releasing hormone-secreting neurons. J. Clin. Endocr. Metab. 85: 1658-1665, 2000.
Okafor, M. C.; Delamere, N. A.: The inhibitory influence of endothelin on active sodium-potassium transport in porcine lens. Invest. Ophthal. Vis. Sci. 42: 1018-1023, 2001.
Zeidel, M. L.; Brady, H. R.; Kone, B. C.; Gullans, S. R.; Brenner, B. M.: Endothelin, a peptide inhibitor of Na(+)-K(+)-ATPase in intactrenal tubular epithelial cells. Am. J. Physiol. 257: C1101-C1107, 1989.
Lerman, C.; Caporaso, N. E.; Audrain, J.; Main, D.; Bowman, E. D.; Lockshin, B.; Boyd, N. R.; Shields, P. G.: Evidence suggesting the role of specific genetic factors in cigarette smoking. Health Psych. 18: 14-20, 1999.
Lossie, A. C.; Vandenbergh, D. J.; Uhl, G. R.; Camper, S. A.: Localization of the dopamine transporter gene, Dat1, on mouse chromosome
Mammalian Genome 5: 117-118, 1994.13. Sabol, S. Z.; Nelson, M. L.; Fisher, C.; Gunzerath, L.; Brody, C. L.; Hu, S.; Sirota, L. A.; Marcus, S. E.; Greenberg, B. D.; Lucas, F. R., IV; Benjamin, J.; Murphy, D. L.; Hamer, D. H.: A genetic association for cigarette smoking behavior. Health Psych. 18: 7-13, 1999.
Tiihonen, J.; Kuikka, J.; Bergstrom, K.; Hakola, P.; Karhu, J.; Ryynanen, O.-P.; Fohr, J.: Altered striatal dopamine reuptake site densities in habitually violent and non-violent alcoholics. Nature Med. 1: 654-657, 1995.
Vandenbergh, D. J.; Persico, A. M.; Hawkins, A. L.; Griffin, C. A.; Li, X.; Jabs, E. W.; Uhl, G. R.: Human dopamine transporter gene (DAT1) maps to chromosome 5p15.3 and displays a VNTR. Genomics 14:1104-1106, 1992.
Vandenbergh, D. J.; Persico, A. M.; Uhl, G. R.: A human dopamine transporter cDNA predicts reduced glycosylation, displays a novel repetitive element and provides racially-dimorphic TaqI RFLPs. Molec. Brain Res. 15: 161-166, 1992.
Waldman, I. D.; Rowe, D. C.; Abramowitz, A.; Kozel, S. T.; Mohr, J. H.; Sherman, S. L.; Cleveland, H. H.; Sanders, M. L.; Gard, J. M. C.; Stever, C.: Association and linkage of the dopamine transporter gene and attention-deficit hyperactivity disorder in children: heterogeneity owing to diagnostic subtype and severity. Am. J. Hum. Genet. 63:1767-1776, 1998.
von Boehmer, H.; Sarukhan, A.: GAD, a single autoantigen for diabetes. Science 284: 1135-1136, 1999.
McGill, G. G.; Horstmann, M.; Widlund, H. R.; Du, J.; Motyckova, G.; Nishimura, E. K.; Lin, Y.-L.; Ramaswamy, S.; Avery, W.; Ding, H.-F.; Jordan, S. A.; Jackson, I. J.; Korsmeyer, S. J.; Golub, T. R.; Fisher, D. E.: Bcl2 regulation by the melanocyte master regulator Mitf modulates lineage survival and melanoma cell viability. Cell 109:707-718, 2002
Geck, P.; Sonnenschein, C.; Soto, A. M.: The D13S171 marker, misannotated to BRCA2, links the AS3 gene to various cancers. (Letter) Am. J. Hum. Genet. 69: 461-463, 2001.
Geck, P.; Szelei, J.; Jimenez, J.; Sonnenschein, C.; Soto, A. M.: Early gene expression during androgen-induced inhibition of proliferation of prostate cancer cells: a new suppressor candidate on chromosome 13, in the BRCA2-Rb1 locus. J. Steroid Biochem. Molec. Biol. 68:41-50, 1999.
Okazaki, I.; Kinoshita, K.; Muramatsu, M.; Yoshikawa, K.; Honjo, T.: The AID enzyme induces class switch recombination in fibroblasts. Nature 416:340-345, 2002.
Petersen-Mahrt, S. K.; Harris, R. S.; Neuberger, M. S.: AID mutates E. coli suggesting a DNA deamination mechanism for antibody diversification. Nature 418:99-104, 2002.
Revy, P.; Muto, T.; Levy, Y.; Geissmann, F.; Plebani, A.; Sanal, O.; Catalan, N.; Forveille, M.; Dufourcq-Lagelouse, R.; Gennery, A.; Tezcan, I.; Ersoy, F.; and 9 others: Activation-induced cytidine deaminase (AID) deficiency causes the autosomal recessive form of the hyper-IgM syndrome (HIGM2). Cell 102: 565-575, 2000.
Yoshikawa, K.; Okazaki, I.; Eto, T.; Kinoshita, K.; Muramatsu, M.; Nagaoka, H.; Honjo, T.: AID enzyme-induced hypermutation in an actively transcribed gene in fibroblasts. Science 296: 2033-2036, 2002.
Tamari, M.; Daigo, Y.; Nakamura, Y.: Isolation and characterization of a novel serine threonine kinase gene on chromosome 3q22-21.3. J. Hum. Genet. 44: 116-120, 1999.
Tamari, M.; Daigo, Y.; Ishikawa, S.; Nakamura, Y.: Genomic structure of a novel human gene (XYLB) on chromosome 3p22-p21.3 encoding a xylulo kinase-like protein. Cytogenet. Cell Genet. 82: 101-104, 1998.
Hertzel, A. V.; Bernlohr, D. A.: Cloning and chromosomal location of the murine keratinocyte lipid-binding protein gene. Gene 221:235-243, 1998.
Madsen, P.; Rasmussen, H. H.; Leffers, H.; Honore, B.; Celis, J. E.: Molecular cloning and expression of a novel keratinocyte protein (psoriasis-associated fatty acid-binding protein [PA-FABP]) that is highly up-regulated in psoriatic skin and that shares similarity to fatty acid-binding proteins. J. Invest. Derm. 99: 299-305, 1992.
Siegenthaler, G.; Hotz, R.; Chatellard-Gruaz, D.; Didierjean, L.; Hellman, U.; Saurat, J. H.: Purification and characterization of the human epidermal fatty acid-binding protein: localization during epidermal cell differentiation in vivo and in vitro. Biochem J. 302:363-371, 1994
Polyak, K.; Xia, Y.; Zweier, J. L.; Kinzler, K. W.; Vogelstein, B.: A model for p53-induced apoptosis. Nature 389: 300-305, 1997.
Hernandez, M.-C.; Andres-Barquin, P. J.; Holt, I.; Israel, M. A.: Cloning of human ENC-1 and evaluation of its expression and regulation in nervous system tumors. Exp. Cell Res. 242: 470-477, 1998.
Hernandez, M.-C.; Andres-Barquin, P. J.; Israel, M. A.: Assignment of the ectodermal-neural cortex 1 gene (Enc1) to mouse chromosome band 13D1 by fluorescence in situ hybridization. Cytogenet. Cell Genet. 89: 158-159, 2000.
Hernandez, M.-C.; Andres-Barquin, P. J.; Kuo, W. L.; Israel, M. A.: Assignment of the ectodermal-neural cortex 1 gene (ENC1) to human chromosome band 5q13 by in situ hybridization. Cytogenet. Cell Genet. 87:89-90, 1999.
Kim, T.-A.; Lim, J.; Ota, S.; Raja, S.; Rogers, R.; Rivnay, B.; Avraham, H.; Avraham, S.: NRP/B, a novel nuclear matrix protein, associates with p110(RB) and is involved in neuronal differentiation. J. Cell Biol. 141: 553-566, 1998.
Christopoulos, G.; Perry, K. J.; Morfis, M.; Tilakaratne, N.; Gao, Y.; Fraser, N. J.; Main, M. J.; Foord, S. M.; Sexton, P. M.: Multiple amylin receptors arise from receptor activity-modifying protein interaction with the calcitonin receptor gene product. Molec. Pharm. 56: 235-242, 1999.
de Vernejoul, M.-C.: Personal Communication. Paris, France Jan. 19, 1999.
Gorn, A. H.; Lin, H. Y.; Yamin, M.; Auron, P. E.; Flannery, M. R.; Tapp, D. R.; Manning, C. A.; Lodish, H. F.; Krane, S. M.; Goldring, S. R.: Cloning, characterization, and expression of a human calcitonin receptor from an ovarian carcinoma cell line. J. Clin. Invest. 90:1726-1735, 1992.
Bundey, S.: Recent views on genetic factors in retinoblastoma. (Abstract) J. Med. Genet. 17: 386-387, 1980.
Bundey, S.; Morten, J. E. N.: An unusual pedigree with retinoblastoma. Does it shed light on the delayed mutation and host resistance theories? Hum. Genet. 59: 434-436, 1981.
Cance, W. G.; Brennan, M. F.; Dudas, M. E.; Huang, C.-M.; Cordon-Cardo, C.: Altered expression of the retinoblastoma gene product in human sarcomas. New Eng. J. Med. 323: 1457-1462, 1990.
Canning, S.; Dryja, T. P.: Short, direct repeats at the breakpoints of deletions of the retinoblastoma gene. Proc. Nat. Acad. Sci. 86:5044-5048, 1989.
Carlson, E. A.; Desnick, R. J.: Mutational mosaicism and genetic counseling in retinoblastoma. Am. J. Med. Genet. 4: 365-381, 1979.
Cavenee, W. K.: The genetic basis of neoplasia: the retinoblastoma paradigm. Trends Genet. 2: 299-300, 1986.
Cavenee, W. K.; Dryja, T. P.; Phillips, R. A.; Benedict, W. F.; Godbout, R.; Gallie, B. L.; Murphree, A. L.; Strong, L. C.; White, R. L.: Expression of recessive alleles by chromosomal mechanisms in retinoblastoma. Nature 305: 779-784, 1983.
Cavenee, W. K.; Hansen, M. F.; Nordenskjold, M.; Kock, E.; Maumenee, I.; Squire, J. A.; Phillips, R. A.; Gallie, B. L.: Genetic origin of mutations predisposing to retinoblastoma. Science 228: 501-503, 1985.
Cavenee, W. K.; Murphree, A. L.; Shull, M. M.; Benedict, W. F.; Sparkes, R. S.; Kock, E.; Nordenskjold, M.: Prediction of familial predisposition to retinoblastoma. New Eng. J. Med. 314: 1201-1207, 1986.
Chano, T.; Ikegawa, S.; Kontani, K.; Okabe, H.; Baldini, N.; Saeki, Y.: Identification of RB1CC1, a novel human gene that can induce RB1 in various human cells. Oncogene 21: 1295-1298, 2002.
Chauveinc, L.; Mosseri, V.; Quintana, E.; Desjardins, L.; Schlienger, P.; Doz, F.; Dutrillaux, B.: Osteosarcoma following retinoblastoma: age at onset and latency period. Ophthalmic Genet. 22: 77-88, 2001.
Chen, P.-L.; Scully, P.; Shew, J.-Y.; Wang, J. Y. J.; Lee, W.-H.: Phosphorylation of the retinoblastoma gene product is modulated during the cell cycle and cellular differentiation. Cell 58: 1193-1198, 1989.
Connolly, M. J.; Payne, R. H.; Johnson, G.; Gallie, B. L.; Allderdice, P. W.; Marshall, W. H.; Lawton, R. D.: Familial, EsD-linked, retinoblastoma with reduced penetrance and variable expressivity. Hum. Genet. 65:122-124, 1983.
Cowell, J. K.; Bia, B.: A novel missense mutation in patients from a retinoblastoma pedigree showing only mild expression of the tumor phenotype. Oncogene 16: 3211-3213, 1998.
Cowell, J. K.; Rutland, P.; Hungerford, J.; Jay, M.: Deletion of chromosome region 13q14 is transmissible and does not always predispose to retinoblastoma. Hum. Genet. 80: 43-45, 1988.
Cowell, J. K.; Rutland, P.; Jay, M.; Hungerford, J.: Deletions of the esterase D locus from a survey of 200 retinoblastoma patients. Hum. Genet. 72: 164-167, 1986.
Cowell, J. K.; Smith, T.; Bia, B.: Frequent constitutional C to T mutations in CGA-arginine codons in the RB1 gene produce premature stop codons in patients with bilateral (hereditary) retinoblastoma. Europ. J. Hum. Genet. 2: 281-290, 1994.
Dahiya, A.; Wong, S.; Gonzalo, S.; Gavin, M.; Dean, D. C.: Linking the Rb and Polycomb pathways. Molec. Cell 8: 557-568, 2001.
Davison, E. V.; Gibbons, B.; Aherne, G. E. S.; Roberts, D. F.: Chromosomes in retinoblastoma patients. Clin. Genet. 15: 505-508, 1979.
DeCaprio, J. A.; Ludlow, J. W.; Lynch, D.; Furukawa, Y.; Griffin, J.; Piwnica-Worms, H.; Huang, C.-M.; Livingston, D. M.: The product of the retinoblastoma susceptibility gene has properties of a cell cycle regulatory element. Cell 58: 1085-1095, 1989.
de Grouchy, J.; Turleau, C.; Cabanis, M. O.; Richardet, J. M.: Retinoblastome et deletion intercalaire du chromosome 13. Arch. Franc. Pediat. 37: 531-535, 1980.
Dryja, T.; Cavenee, W.; Epstein, J.; Rapaport, J.; Goorin, A.; Koufos, A.: Chromosome 13 homozygosity in osteogenic sarcoma without retinoblastoma. (Abstract) Am. J. Hum. Genet. 36: 28S, 1984.
Dryja, T. P.; Bruns, G. A. P.; Gallie, B.; Petersen, R.; Green, W.; Rapaport, J. M.; Albert, D. M.; Gerald, P. S.: Low incidence of deletion of the esterase D locus in retinoblastoma patients. Hum. Genet. 64: 151-155, 1983.
Dryja, T. P.; Cavenee, W.; White, R.; Rapaport, J. M.; Petersen, R.; Albert, D. M.; Bruns, G. A. P.: Homozygosity of chromosome 13 in retinoblastoma. New Eng. J. Med. 310: 550-553, 1984.
Dryja, T. P.; Friend, S.; Weinberg, R. A.: Isolation of a cDNA fragment derived from human retina mRNA which detects a locus within 13q14 often deleted in retinoblastomas. (Abstract) Am. J. Hum. Genet. 39: A29, 1986.
Dryja, T. P.; Mukai, S.; Petersen, R.; Rapaport, J. M.; Walton, D.; Yandell, D. W.: Parental origin of mutations of the retinoblastoma gene. Nature 339: 556-558, 1989.
Dryja, T. P.; Mukai, S.; Rapaport, J. M.; Yandell, D. W.: Parental origin of mutations of the retinoblastoma gene. (Abstract) Am. J. Hum. Genet. 45 (suppl.): A19, 1989.
Clinton, M.; Frangou-Lazaridis, M.; Panneerselvam, C.; Horecker, B. L.: The sequence of human parathymosin deduced from a cloned human kidney cDNA. Biochem. Biophys. Res. Commun. 158: 855-862, 1989.
Szabo, P.; Clinton, M.; Macera, M.; Horecker, B. L.: Localization of the gene coding for parathymosin to chromosome 17 in humans. Cytogenet. Cell Genet. 50: 91-92, 1989.
Carvajal, J. J.; Pook, M. A.; dos Santos, M.; Doudney, K.; Hillermann, R.; Minogue, S.; Williamson, R.; Hsuan, J. J.; Chamberlain, S.: The Friedreich's ataxia gene encodes a novel phosphatidylinositol-4-phosphate5-kinase. Nature Genet. 14: 157-162, 1996.
Carvajal, J. J.; Pook, M. A.; Doudney, K.; Hillermann, R.; Wilkes, D.; A1-Mahdawi, S.; Williamson, R.; Chamberlain, S.:
Friedreich's ataxia: a defect in signal transduction? Hum. Molec. Genet. 4: 1411-1419, 1995.
Pook, M. A.; Carvajal, J. J; Doudney, K.; Hillermann, R.; Chamberlain, S.: Exon-intron structure of a 2.7-kb transcript of the STM7 gene with phosphatidylinositol-4-phosphate 5-kinase activity. Genomics 42:170-172, 1997.
Plougastel, B.; Trowsdale, J.: Cloning of NKG2-F, a new member of the NKG2 family of human natural killer cell receptor genes. Europ. J. Immun. 27: 2835-2839, 1997.
Sutherland, C. L.; Chalupny, N. J.; Schooley, K.; Vanden-Bos, T.; Kubin, M.; Cosman, D.: UL16-binding proteins, novel MHC class I-related proteins, bind to NKG2D and activate multiple signaling pathways in primary NK cells. J. Immun. 168: 671-679, 2002
Wu, J.; Song, Y.; Bakker, A. B. H.; Bauer, S.; Spies, T.; Lanier, L. L.; Phillips, J. H.: An activating immuno receptor complex formed by NKG2D and DAP10. Science 285: 730-732, 1999.
Volkmer, E.; Karnitz, L. M.: Human homologs of Schizosaccharomyces pombe Rad1, Hus1, and Rad9 form a DNA damage-responsive protein complex. J. Biol. Chem. 274: 567-570, 1999.
Hacker, B. M.; Tomlinson, J. E.; Wayman, G. A.; Sultana, R.; Chan, G.; Villacres, E.; Disteche, C.; Storm, D. R.: Cloning, chromosomal mapping, and regulatory properties of the human type 9 adenylyl cyclase (ADCY9). Genomics 50: 97-104, 1998.
Paterson, J. M.; Smith, S. M.; Harmar, A. J.; Antoni, F. A.: Control of a novel adenylyl cyclase by calcineurin. Biochem. Biophys. Res. Commun. 214: 1000-1008, 1995.
Premont, R. T.; Matsuoka, I.; Mattei, M. G.; Pouille, Y.; Defer, N.; Hanoune, J.: Identification and characterization of a widely expressed form of adenylyl cyclase. J. Biol. Chem. 271: 13900-13907, 1996.
Toyota, T.; Hattori, E.; Meerabux, J.; Yamada, K.; Saito, K.; Shibuya, H.; Nankai, M.; Yoshikawa, T.: Molecular analysis, mutation screening, and association study of adenylate cyclase type 9 gene (ADCY9) in mood disorders. Am. J. Med. Genet. (Neuropsychiat. Genet.) 114:84-92, 2002.
Amiel, J.; Salomon, R.; Attie, T.; Pelet, A.; Trang, H.; Mokhtari, M.; Gaultier, C.; Munnich, A.; Lyonnet, S.: Mutations of the RET-GDNF signaling pathway in Ondine's curse. (Letter) Am. J. Hum. Genet. 62:715-717, 1998.
Angrist, M.; Bolk, S.; Thiel, B.; Puffenberger, E. G.; Hofstra, R. M.; Buys, C. H. C. M.; Cass, D. T.; Chakravarti, A.: Mutation analysis of the RET receptor tyrosine kinase in Hirschsprung disease. Hum. Molec. Genet. 4: 821-830, 1995.
Antinolo, G.; Marcos, I.; Fernandez, R. M.; Romero, M.; Borrego, S.: A novel germline point mutation, c.2304G(T, in codon 768 of the RET proto-oncogene in a patient with medullary thyroid carcinoma. (Letter) Am. J. Med. Genet. 110: 85-87, 2002.
Attie, T.; Pelet, A.; Edery, P.; Eng, C.; Mulligan, L. M.; Amiel, J.; Boutrand, L.; Beldjord, C.; Nihoul-Fekete, C.; Munnich, A.; Ponder, B. A. J.; Lyonnet, S.: Diversity of RET proto-oncogene mutations in familial and sporadic Hirschsprung disease. Hum. Molec. Genet. 4:1381-1386, 1995.
Attie-Bitach, T.; Abitbol, M.; Gerard, M.; Delezoide, A.-L.; Auge, J.; Pelet, A.; Amiel, J.; Pachnis, V.; Munnich, A.; Lyonnet, S.; Vekemans, M.: Expression of the RET protooncogene in human embryos. Am. J. Med. Genet. 80: 481-486, 1998.
Batourina, E.; Choi, C.; Paragas, N.; Bello, N.; Hensle, T.; Costantini, F. D.; Schuchardt, A.; Bacallao, R. L.; Mendelsohn, C. L.: Distalureter morphogenesis depends on epithelial cell remodeling mediated by vitamin A and Ret. Nature Genet. 32: 109-115, 2002. Note: Erratum: Nature Genet. 32: 331 only, 2002.
Batourina, E.; et al; et al: Vitamin A controls epithelial/mesenchymal interactions through Ret expression. Nature Genet. 27: 74-78, 2001.
Berndt, I.; Reuter, M.; Saller, B.; Frank-Raue, K.; Groth, P.: Grubendorf, M.; Raue, F.; Ritter, M. M.; Hoppner, W.: A new hot spot for mutations in the ret protooncogene causing familial medullary thyroid carcinoma and multiple endocrine neoplasia type 2A. J. Clin. Endocr. Metab. 83: 770-774, 1998.
Auricchio, A.; Griseri, P.; Carpentieri, M. L.; Betsos, N.; Staiano, A.; Tozzi, A.; Priolo, M.; Thompson, H.; Bocciardi, R.; Romeo, G.; Ballabio, A.; Ceccherini, I.: Double heterozygosity for a RET substitution interfering with splicing and an EDNRB missense mutation in Hirschsprung disease. (Letter) Am. J. Hum. Genet. 64: 1216-1221, 1999.
Boccia, L. M.; Green, J. S.; Joyce, C.; Eng, C.; Taylor, S. A. M.; Mulligan, L. M.: Mutation of RET codon 768 is associated with the FMTC phenotype. Clin. Genet. 51: 81-85, 1997.
Bolino, A.; Schuffenecker, I.; Luo, Y.; Seri, M.; Silengo, M.; Tocco, T.; Chabrier, G.; Houdent, C.; Murat, A.; Schlumberger, M.; Tourniaire, J.; Lenoir, G. M.; Romeo, G.: RET mutations in exons 13 and 14 of FMTC patients. Oncogene 10: 2415-2419, 1995.
Bolk, S.; Angrist, M.; Schwartz, S.; Silvestri, J. M.; Weese-Mayer, D. E.; Chakravarti, A.: Congenital central hypoventilation syndrome: mutation analysis of the receptor tyrosine kinase RET. Am. J. Med. Genet. 63: 603-609, 1996.
Bolk Gabriel, S.; Salomon, R.; Pelet, A.; Angrist, M.; Amiel, J.; Formage, M.; Attie-Bitach, T.; Olson, J. M.; Hofstra, R.; Buys, C.; Steffann, J.; Munnich, A.; Lyonnet, S.; Chakravarti, A.: Segregation at three loci explains familial and population risk in Hirschsprung disease. Nature Genet. 31: 89-93, 2002.
Ceccherini, I.; Hofstra, R. M.; Luo, Y.; Stulp, R. P.; Barone, V.; Stelwagen, T.; Bocciardi, R.; Nijveen, H.; Bolino, A.; Seri, M.; Ronchetto, P.; Pasini, B.; Bozzano, M.; Buys, C. H. C. M.; Romeo, G.: DNA polymorphisms and conditions for SSCP analysis of the 20 exons of the Ret protooncogene. Oncogene 9: 3025-3029, 1994.
Navab, M.; Hama-Levy, S.; Van Lenten, B. J.; Fonarow, G. C.; Cardinez, C. J.; Castellani, L. W.; Brennan, M.-L.; Lusis, A. J.; Fogelman, A. M.: Mildly oxidized LDL induces an increased apolipoprotein J/paraoxonase ratio. J. Clin. Invest. 99: 2005-2019, 1997.
Houchins, J. P.; Yabe, T.; McSherry, C.; Bach, F. H.: DNA sequence analysis of NKG2, a family of related cDNA clones encoding type II integral membrane proteins on human natural killer cells. J. Exp. Med. 173: 1017-1020, 1991.
Plougastel, B.; Trowsdale, J.: Sequence analysis of a 62-kb region overlapping the human KLRC cluster of genes. Genomics 49: 193-199, 1998.
Yabe, T.; McSherry, C.; Bach, F. H.; Fisch, P.; Schall, R. P.; Sondel, P. M.; Houchins, J. P.: A multi gene family on human chromosome 12 encodes natural killer-cell lectins. Immunogenetics 37: 455-460, 1993.
Rousseau-Merck, M.-F.; Zahraoui, A.; Touchot, N.; Tavitian, A.; Berger, R.: Chromosome assignment of four RAS-related RAB genes. Hum. Genet. 86: 350-354, 1991.
Li, F. P.; Decker, H.-J. H.; Zbar, B.; Stanton, V. P., Jr.; Kovacs, G.; Seizinger, B. R.; Aburatani, H.; Sandberg, A. A.; Berg, S.; Hosoe, S.; Brown, R. S.: Clinical and genetic studies of renal cell carcinomas in a family with a constitutional chromosome 3;8 translocation: genetics of familial renal carcinoma. Ann. Intern. Med. 118: 106-111, 1993.
Ohta, M.; Inoue, H.; Cotticelli, M. G.; Kastury, K.; Baffa, R.; Palazzo, J.; Siprashvili, Z.; Mori, M.; McCue, P.; Druck, T.; Croce, C. M.; Huebner, K.: The FHIT gene, spanning the chromosome 3p14.2 fragile site and renal carcinoma-associated t(3;8) breakpoint, is abnormal in digestive tract cancers. Cell 84: 587-597, 1996.
Puffenberger, E. G.; Hosoda, K.; Washington, S. S.; Nakao, K.; de Wit, D.; Yanagisawa, M.; Chakravarti, A.: A missense mutation of the endothelin-B receptor gene in multigenic Hirschsprung's disease. Cell 79:1257-1266, 1994.
Svensson, P.-J.; Anvret, M.; Molander, M.-L.; Nordenskjold, A.: Phenotypic variation in a family with mutations in two Hirschsprung-related genes (RET and endothelin receptor B). Hum. Genet. 103: 145-148, 1998.
Hanks, M.; Wurst, W.; Anson-Cartwright, L.; Auerbach, A. B.; Joyner, A. L.: Rescue of the En-1 mutant phenotype by replacement of En-1 with En-2. Science 269: 679-682, 1995.
Johnson, R. L.; Tabin, C. J.: Molecular models for vertebrate limb development. Cell 90: 979-990, 1997.
Salmeen, A.; Andersen, J. N.; Myers, M. P.; Tonks, N. K.; Barford, D.: Molecular basis for the dephosphorylation of the activation segment of the insulin receptor by protein tyrosine phosphatase 1B. Molec. Cell 6: 1401-1412, 2000.
Polymeropoulos, M. H.; Torres, R.; Yanovski, J. A.; Chandrasekharappa, S. C.; Ledbetter, D. H.: The human corticotropin-releasing factor receptor (CRHR) gene maps to chromosome 17q12-q22. Genomics 28:123-124, 1995.
Sakai, K.; Yamada, M.; Horiba, N.; Wakui, M.; Demura, H.; Suda, T.: The genomic organization of the human corticotropin-releasing factor type-1 receptor. Gene 219: 125-130, 1998.
Sillaber, I.; Rammes, G.; Zimmermann, S.; Mahal, B.; Zieglgansberger, W.; Wurst, W.; Holsboer, F.; Spanagel, R.: Enhanced and delayed stress-induced alcohol drinking in mice lacking functional CRH1 receptors. Science 296:931-933, 2002.
Smith, G. W.; Aubry, J.-M.; Dellu, F.; Contarino, A.; Bilezikjian, L. M.; Gold, L. H.; Chen, R.; Marchuk, Y.;
Hauser, C.; Bentley, C. A.; Sawchenko, P. E.; Koob, G. F.; Vale, W.; Lee, K.-F.: Corticotropin releasing factor receptor 1-deficient mice display decreased anxiety, impaired stress response, and aberrant neuroendocrine development. Neuron 20:1093-1102, 1998.
Timpl, P.; Spanagel, R.; Sillaber, I.; Kresse, A.; Reul, J. M. H. M.; Stalla, G. K.; Blanquet, V.; Steckler, T.; Holsboer, F.; Wurst, W.: Impaired stress response and reduced anxiety in mice lacking a functional corticotropin-releasing hormone receptor 1. Nature Genet. 19:162-166, 1998.
Brissenden, J. E.; Derynck, R.; Francke, U.: Transforming growth factor alpha gene (TGFA) maps to human chromosome 2 close to the breakpoint of the t(2;8) variant translocation in Burkitt lymphoma. (Abstract) Cytogenet. Cell Genet. 40: 589 only, 1985.
Collin, G. B.; Marshall, J. D.; Naggert, J. K.; Nishina, P. M.: TGFA exon-intron structure and evaluation as a candidate gene for Alstrom syndrome. (Letter) Clin. Genet. 55: 61-62, 1999.
Ellis, D. L.; Kafka, S. P.; Chow, J. C.; Nanney, L. B.; Inman, W. H.; McCadden, M. E.; King, L. E., Jr.: Melanoma, growth factors, acanthosis nigricans, the sign of Leser-Trelat, and multiple acrochordons: a possible role for alpha-transforming growth factor in cutaneousparaneo-plastic syndromes. New Eng. J. Med. 317: 1582-1587, 1987.
Fernandez-Larrea, J.; Merlos-Suarez, A.; Urena, J. M.; Baselga, J.; Arribas, J.: A role for a PDZ protein in the early secretory pathway for the targeting of proTGF-alpha to the cell surface. Molec. Cell 3: 423-433, 1999.
Fowler, K. J.; Mann, G. B.; Dunn, A. R.: Linkage of the murine transforming growth factor-alpha gene with Igk, Ly-2, and Fabp1 on chromosome 6. Genomics 16: 782-784, 1993.
Tam, J. P.; Scheikh, M. A.; Solomon, D. S.; Ossowski, L.: Efficient synthesis of human type alpha transforming growth factor: its physical and biological characterization. Proc. Nat. Acad. Sci. 83: 8082-8086, 1986.
Tricoli, J. V.; Nakai, H.; Byers, M. G.; Rall, L. B.; Bell, G. I.; Shows, T. B.: Assignment of the gene coding for human TGF-alpha to chromosome 2p13. (Abstract) Cytogenet. Cell Genet. 40: 762 only, 1985.
Tricoli, J. V.; Nakai, H.; Byers, M. G.; Rall, L. B.; Bell, G. I.; Shows, T. B.: The gene for human transforming growth factor alpha is on the short arm of chromosome 2. Cytogenet. Cell Genet. 42:94-98, 1986
Kikuchi, S.; Hata, M.; Fukumoto, K.; Yamane, Y.; Matsui, T.; Tamura, A.; Yonemura, S.; Yamagishi, H.; Keppler, D.; Tsukita, S.; Tsukita, S.: Radixin deficiency causes conjugated hyperbilirubinemia with loss of Mrp2 from bile canalicular membranes. Nature Genet. 31:320-325, 2002.
Wilgenbus, K. K.; Milatovich, A.; Francke, U.; Furthmayr, H.: Molecular cloning, cDNA sequence, and chromosomal assignment of the human radixin gene and two dispersed pseudo genes. Genomics 16: 199-206, 1993.
Zahraoui, A.; Touchot, N.; Chardin, P.; Tavitian, A.: The human rab genes encode a family of GTP-binding proteins related to yeast YPT1 and SEC4 products involved in secretion. J. Biol. Chem. 264:12394-12401, 1989.
Shimizu, A.; Sakai, Y.; Ohno, K.; Masaki, S.; Kuwano, R.; Takahashi, Y.; Miyashita, N.; Watanabe, T.: A molecular genetic linkage map of mouse chromosome 10, including the Myb, S100b, Pah, SI, and Ifg genes. Biochem. Genet. 30: 529-535, 1992.
Tan, M.; Jing, T.; Lan, K.-H.; Neal, C. L.; Li, P.; Lee, S.; Fang, D.; Nagata, Y.; Liu, J.; Arlinghaus, R.; Hung, M.-C.; Yu, D.: Phosphorylation on tyrosine-15 of p34(Cdc2) by ErbB2 inhibits p34(Cdc2) activation and is involved in resistance to taxol-induced apoptosis. Molec. Cell 9: 993-1004, 2002.
Spicer, A. P.; Seldin, M. F.; Olsen, A. S.; Brown, N.; Wells, D. E.; Doggett, N. A.; Itano, N.; Kimata, K.; Inazawa, J.; McDonald, J. A.: Chromosomal localization of the human and mouse hyaluronan synthase genes. Genomics 41: 493-497, 1997.
Watanabe, K.; Yamaguchi, Y.: Molecular identification of a putative human hyaluronan synthase. J. Biol. Chem. 271: 22945-22948, 1996.
Cahill, D. P.; da Costa, L. T.; Carson-Walter, E. B.; Kinzler, K. W.; Vogelstein, B.; Lengauer, C.: Characterization of MAD2B and other mitotic spindle checkpoint genes. Genomics 58: 181-187, 1999.
Chen, R.-H.; Waters, J. C.; Salmon, E. D.; Murray, A. W.: Association of spindle assembly checkpoint component XMAD2 with unattached kinetochores. Science 274:242-245, 1996.
Dobles, M.; Liberal, V.; Scott, M. L.; Benezra, R.; Sorger, P. K.: Chromosome missegregation and apoptosis in mice lacking the mitotic checkpoint protein Mad2. Cell 101: 635-645, 2000.
Krishnan, R.; Goodman, B.; Jin, D.-Y.; Jeang, K.-T.; Collins, C.; Stetten, G.; Spencer, F.: Map location and gene structure of the Homo sapiens mitotic arrest deficient 2 (MAD2L1) gene at 4q27. Genomics 49:475-478, 1998.
Li, X.; Nicklas, R. B.: Mitotic forces control a cell-cycle checkpoint. Nature 373:630-632, 1995.
Li, Y.; Benezra, R.: Identification of a human mitotic checkpoint gene: hsMAD2. Science 274: 246-248, 1996.
Luo, X.; Tang, Z.; Rizo, J.; Yu, H.: The Mad2 spindle checkpoint protein undergoes similar major conformational changes upon binding to either Mad1 or Cdc20. Molec. Cell 9: 59-71, 2002.
Michel, L. S.; Liberal, V.; Chatterjee, A.; Kirchwegger, R.; Pasche, B.; Gerald, W.; Dobles, M.; Sorger, P. K.; Murty, V. V. V. S.; Benezra, R.: MAD2 haplo-insufficiency causes premature anaphase and chromosome instability in mammalian cells. Nature 409: 355-359, 2001.
Nelson, K. K.; Schlondorff, J.; Blobel, C. P.: Evidence for an interaction of the metalloprotease-disintegrin tumour necrosis factor alpha convertase (TACE) with mitotic arrest deficient 2 (MAD2), and of the metalloprotease-disintegrin MDC9 with a novel MAD2-related protein, MAD2-beta. Biochem. J. 343: 673-680, 1999.
Shonn, M. A.; McCarroll, R.; Murray, A. W.: Requirement of the spindle checkpoint for proper chromosome segregation in budding yeast meiosis. Science 289: 300-303, 2000.
Xu, L.; Deng, H. X.; Yang, Y.; Xia, J. H.; Hung, W. Y.; Siddque, T.: Assignment of mitotic arrest deficient protein 2 (MAD2L1) to human chromosome band 5q23.3 by in situ hybridization. Cytogenet. Cell Genet. 78: 63-64, 1997.
Liu, Y.; Chiu, I.-M.: Assignment of FGF12, the human FGF homologous factor 1 gene, to chromosome 3q29-3qter by fluorescence in situ hybridization. Cytogenet. Cell Genet. 78: 48-49, 1997.
Caslini, C.; Spinelli, O.; Cazzaniga, G.; Golay, J.; De Gioia, L.; Pedretti, A.; Breviario, F.; Amaru, R.; Barbui, T.; Biondi, A.; Introna, M.; Rambaldi, A.: Identification of two novel isoforms of the ZNF162 gene: a growing family of signal transduction and activator of RNA proteins. Genomics 42: 268-277, 1997.
Kramer, A.; Quentin, M.; Mulhauser, F.: Diverse modes of alternative splicing of human splicing factor SF1 deduced from the exon-intron structure of the gene. Gene 211: 29-37, 1998.
Imhof, M. O.; McDonnell, D. P.: Yeast RSP5 and its human homologh RPF1 potentiate hormone-dependent activation of transcription by human progesterone and glucocorticoid receptors. Molec. Cell. Biol. 16:2594-2605, 1996.
Kumar, S.; Harvey, K. F.; Kinoshita, M.; Copeland, N. G.; Noda, M.; Jenkins, N. A.: cDNA cloning, expression analysis, and mapping of the mouse Nedd4 gene. Genomics 40: 435-443, 1997.
Bennett, E. P.; Hassan, H,.; Clausen, H.: cDNA cloning and expression of a novel human UDP-N-acetyl-alpha-D-galactosamine. J. Biol. Chem. 271:17006-17012, 1996.
Bennett, E. P.; Weghuis, D. O.; Merkx, G.; Geurts van Kessel, A.; Eiberg, H.; Clausen, H.: Genomic organization and chromosomal localization of three members of the UDP-N-acetylgalactosamine: polypeptide N-acetylgalactosaminyltransferase family. Glycobiology 8: 547-555, 1998.
Zara, J.; Hagen, F. K.; Ten Hagen, K. G.; Van Wuyckhuyse, B. C.; Tabak, L. A.: Cloning and expression of mouse UDP-Gal NAc: polypeptide-N-acetylgalactosaminyltransferase-T3. Biochem. Biophys. Res. Commun. 228:38-44, 1996.
Blake, D. J.; Nawrotzki, R.; Loh, N. Y.; Gorecki, D. C.; Davies, K. E.: Beta-dystrobrevin, a member of the dystrophin-related protein family. Proc. Nat. Acad. Sci. 95: 241-246, 1998
England, S. K.; Uebele, V. N.; Shear, H.; Kodali, J.; Bennett, P. B.; Tamkun, M. M.: Characterization of a voltage-gated K+ channel beta subunit expressed in human heart. Proc. Nat. Acad. Sci. 92:6309-6313, 1995.
Jones, J. M.; Bentley, E.; Meisler, M. H.; Darling, S. M.: Genetic mapping of the voltage-gated shaker potassium channel beta subunit Kcnab1 to mouse chromosome 3. Mammalian Genome 9: 260 only, 1998.
Leicher, T.; Bahring, R.; Isbrandt, D.; Pongs, O.: Coexpression of the KCNA3B gene product with Kv1.5 leads to a novel A-type potassium channel. J. Biol. Chem. 273: 35095-35101, 1998.
Leicher, T. Roeper, J.; Weber, K.; Wang, X.; Pongs, O.: Structural and functional characterization of human potassium channel subunit beta-1 (KCNA1B). Neuropharmacology 35: 787-795, 1996.
McCormack, K.; McCormack, T.; Tanouye, M.; Rudy, B.; Stuhmer, W.: Alternative splicing of the human Shaker K+ channel beta-1 gene and functional expression of the beta-2 gene product. FEBS Lett. 370:32-36, 1995.
Schultz, D.; Litt, M.; Smith, L.; Thayer, M.; McCormack, K.: Localization of two potassium channel beta subunit genes, KCNA1B and KCNA2B. Genomics 31:389-391, 1996
Kanai, N.; Lu, R.; Satriano, J. A.; Bao, Y.; Wolkoff, A. W.; Schuster, V. L.: Identification and characterization of a prostaglandin transporter. Science 268:866-869, 1995.
Lu, R.; Kanai, N.; Bao, Y.; Schuster, V. L.: Cloning, in vitro expression, and tissue distribution of a human prostaglandin transporter cDNA (hPGT). J. Clin. Invest. 98: 1142-1149, 1996.
Lu, R.; Schuster, V. L.: Molecular cloning of the gene for the human prostaglandin transporter hPGT: gene organization, promoter activity, and chromosomal localization. Biochem. Biophys. Res. Commun. 246:805-812, 1998.
Loughna, S.; Sato, T. N.: A combinatorial role of angiopoietin-1 and orphan receptor TIE1 pathways in establishing vascular polarity during angiogenesis. Molec. Cell 7: 233-239, 2001.
Yu, H.; Peters, J.-M.; King, R. W.; Page, A. M.; Hieter, P.; Kirschner, M. W.: Identification of a cullin homology region in a subunit of the anaphase-promoting complex. Science 279: 1219-1222, 1998.
Zhao, N.; Lai, F.; Fernald, A. A.; Eisenbart, J. D.; Espinosa, R., III.; Wang, P. W.; Le Beau, M. M.: Human CDC23: cDNA cloning, mapping to 5q31, genomic structure, and evaluation as a candidate tumor suppressor gene in myeloid leukemias. Genomics 53: 184-190, 1998.
Brambilla, R.; Draetta, G.: Molecular cloning of PISSLRE, a novel putative member of the cdk family of protein serine/threonine kinases. Oncogene 9:3037-3041, 1994.
Grana, X.; Claudio, P. P.; De Luca, A.; Sang, N.; Giordano, A.: PISSLRE, a human novel CDC2-related protein kinase. Oncogene 9:2097-2103, 1994.
Deak, M.; Clifton, A. D.; Lucocq, J. M.; Alessi, D. R.: Mitogen- and stress-activated protein kinase-1 (MSK1) is directly activated by MAPK and SAPK2/p38, and may mediate activation of CREB. EMBO J. 17:4426-4441, 1998.
Jiang, C.; Yu, L.; Tu, Q.; Zhao, Y.; Zhang, H.; Zhao, S.: Assignment of a member of the ribosomal protein S6 kinase family, RPS6KA5, to human chromosome 14q31-q32.1 by radiation hybrid mapping. Cytogenet. Cell Genet. 87: 261-261, 1999.
Marsters, S. A.; Sheridan, J. P.; Pitti, R. M.; Huang, A.; Skubatch, M.; Baldwin, D.; Yuan, J.; Gurney, A.; Goddard, A. D.; Godowski, P.; Ashkenazi, A.: A novel receptor for Apo2L/TRAIL contains a truncated death domain. Curr. Biol. 7: 1003-1006, 1997.
MacFarlane, M.; Ahmad, M.; Srinivasula, S. M.; Fernandes-Alnemri, T.; Cohen, G. M.; Alnemri, E. S.: Identification and molecular cloning of two novel receptors for the cytotoxic ligand TRAIL. J. Biol. Chem. 272:25417-25420, 1997.
Pai, S. I.; Wu, G. S.; Ozoren, N.; Wu, L.; Jen, J.; Sidransky, D.; El-Deiry, W. S.: Rare loss-of-function mutation of a death receptor gene in head and neck cancer. Cancer Res. 58: 3513-3518, 1998.
Pan, G.; Ni, J.; Wei, Y.-F.; Yu, G.; Gentz, R.; Dixit, V. M.: An antagonist decoy receptor and a death domain-containing receptor for TRAIL. Science 277: 815-818, 1997.
Schneider, P.; Bodmer, J.-L.; Thome, M.; Hofmann, K.; Holler, N.; Tschopp, J.: Characterization of two receptors for TRAIL. FEBS Lett. 416:329-334, 1997.
Screaton, G. R.; Mongkolsapaya, J.; Xu, X.-N.; Cowper, A. E.; McMichael, A. J.; Bell, J. I.: TRICK2, a new alternatively spliced receptor that transduces the cytotoxic signal from TRAIL. Curr. Biol. 7:693-696, 1997.
Sheridan, J. P.; Marsters, S. A.; Pitti, R. M.; Gurney, A.; Skubatch, M.; Baldwin, D.; Ramakrishnan, L.; Gray, C. L.; Baker, K.; Wood, W. I.; Goddard, A. D.; Godowski, P.; Ashkenazi, A.: Control of TRAIL-induced apoptosis by a family of signaling and decoy receptors. Science 277:818-821, 1997.
Nakamura, T.; Takeuchi, K.; Muraoka, S.; Takezoe, H.; Takahashi, N.; Mori, N.: A neurally enriched coronin-like protein, ClipnC, is a novel candidate for an actin cytoskeleton-cortical membrane-linking protein. J. Biol. Chem. 274: 13322-13327, 1999.
Abbaszade, I.; Liu, R.-Q.; Yang, F.; Rosenfeld, S. A.; Ross, O. H.; Link, J. R.; Ellis, D. M.; Tortorella, M. D.; Pratta, M. A.; Hollis, J. M.; Wynn, R.; Duke, J. L.; and 15 others: Cloning and characterization of ADAMTS11, an aggrecanase from the ADAMTS family. J. Biol. Chem. 274:23443-23450, 1999.
Hagiwara, T.; Tanaka, K.; Takai, S.; Maeno-Hikichi, Y.; Mukainaka, Y.; Wada, K.: Genomic organization, promoter analysis, and chromosomal localization of the gene for the mouse glial high-affinity glutamate transporter Slc1a3. Genomics 33: 508-515, 1996.
Harada, T.; Harada, C.; Watanabe, M.; Inoue, Y.; Sakagawa, T.; Nakayama, N.; Sasaki, S.; Okuyama, S.; Watase, K.; Wada, K.; Tanaka, K.: Functions of the two glutamate transporters GLAST and GLT-1 in the retina. Proc. Nat. Acad. Sci. 95: 4663-4666, 1998.
Keppen, L. D.; Gollin, S. M.; Edwards, D.; Sawyer, J.; Wilson, W.; Overhauser, J.: Clinical phenotype and molecular analysis of a three-generation family with an interstitial deletion of the short arm of chromosome 5. Am. J. Med. Genet. 44: 356-360, 1992.
Kirschner, M. A.; Arriza, J. L.; Copeland, N. G.; Gilbert, D. J.; Jenkins, N. A.; Magenis, E.; Amara, S. G.: The mouse and human excitatory amino acid transporter gene (EAAT1) maps to mouse chromosome 15 and a region of syntenic homology on human chromosome 5. Genomics 22:631-633, 1994.
Shashidharan, P.; Huntley, G. W.; Meyer, T.; Morrison, J. H.; Plaitakis, A.: Neuron-specific human glutamate transporter: molecular cloning, characterization and expression in human brain. Brain Res. 662:245-250, 1994.
Stoffel, W.; Sasse, J.; Duker, M.; Muller, R.; Hofmann, K.; Fink, T.; Lichter, P.: Human high affinity, Na(+)-dependent L-glutamate/L-aspartate transporter GLAST-1 (EAAT-1): gene structure and localization to chromosome 5p11-p12. FEBS Lett. 386: 189-193, 1996.
Takai, S.; Yamada, K.; Kawakami, H.; Tanaka, K.; Nakamura, S.: Localization of the gene (SLC1A3) encoding human glutamate transporter (GluT-1) to 5p13 by fluorescence in situ hybridization. Cytogenet. Cell Genet. 69: 209-210, 1995.
LeClair, K. P.; Rabin, M.; Nesbitt, M. N.; Pravtcheva, D.; Ruddle, F. H.; Palfree, R. G. E.; Bothwell, A.: Murine Ly-6 multi gene family is located on chromosome 15. Proc. Nat. Acad. Sci. 84: 1638-1642, 1987.
Fischer, A.; Durandy, A.; Sterkers, G.; Griscelli, C.: Role of the LFA-1 molecule in cellular interactions required for antibody production in humans. J. Immun. 136: 3198-3203, 1986.
Marlin, S. D.; Morton, C. C.; Anderson, D. C.; Springer, T. A.: LFA-1 immunodeficiency disease: definition of the genetic defect and chromosomal mapping of alpha and beta subunits of the lymphocyte function-associated antigen 1 (LFA-1) by complementation in hybrid cells. J. Exp. Med. 164: 855-867, 1986.
Blackwood, E.; Eisenman, R. N.: Max: a helix-loop-helix zipper protein that forms a sequence-specific DNA-binding complex with Myc. Science 251:1211-1217, 1991.
Eisenman, R. N.: Personal Communication. Seattle, Wash. Jul. 27, 1994.
Gilladoga, A. D.; Edelhoff, S.; Blackwood, E. M.; Eisenman, R. N.; Disteche, C. M.: Mapping of MAX to human chromosome 14 and mouse chromosome 12 by in situ hybridization. Oncogene 7: 1249-1251, 1992
Bulavin, D. V.; Higashimoto, Y.; Popoff, I. J.; Gaarde, W. A.; Basrur, V.; Potapova, O.; Appella, E.; Formace, A. J., Jr.: Initiation of a G2/M checkpoint after ultraviolet radiation requires p38 kinase. Nature 411:102-107, 2001.
Doonan, S.; Barra, D.; Bossa, F.: Structural and genetic relationships between cytosolic and mitochondrial isoenzymes. Int. J. Biochem. 16:1193-1199, 1984.
Copeland, N. G.; Silan, C. M.; Kingsley, D. M.; Jenkins, N. A.; Cannizzaro, L. A.; Croce, C. M.; Huebner, K.; Sims, J. E.: Chromosomal location of murine and human IL-1 receptor genes. Genomics 9: 44-50, 1991.
Dale, M.; Nicklin, M. J.: Interleukin-1 receptor cluster: gene organization of IL1R2, IL1R1, IL1RL2 (IL-1Rrp2), IL1RL1 (T1/ST2), and IL18R1 (IL-1Rrp) on human chromosome 2q. Genomics 57: 177-179, 1999.
Dower, S. K.; Kronheim, S. R.; Hopp, T. P.; Cantrell, M.; Deeley, M.; Gillis, S.; Henney, C. S.; Urdal, D. L.: The cell surface receptors for interleukin-1(alpha) and interleukin-1(beta) are identical. Nature 324:266-268, 1986.
Sims, J. E.; Acres, R. B.; Grubin, C. E.; McMahan, C. J.; Wignall, J. M.; March, C. J.; Dower, S. K.: Cloning the interleukin 1 receptor from human T cells. Proc. Nat. Acad. Sci. 86: 8946-8950, 1989
Justice, M. J.; Siracusa, L. D.; Gilbert, D. J.; Heisterkamp, N.; Groffen, J.; Chada, K.; Silan, C. M.; Copeland, N. G.; Jenkins, N. A.: A genetic linkage map of mouse chromosome 10: localization of eighteen molecular markers using a single interspecific backcross. Genetics 125:855-866, 1990.
Smith, E. P.; Boyd, J.; Frank, G. R.; Takahashi, H.; Cohen, R. M.; Specker, B.; Williams, T. C.; Lubahn, D. B.; Korach, K. S.: Estrogen resistance caused by a mutation in the estrogen-receptor gene in a man. New Eng. J. Med. 331: 1056-1061, 1994.
Costa, R. M.; Federov, N. B.; Kogan, J. H.; Murphy, G. G.; Stern, J.; Ohno, M.; Kucherlapati, R.; Jacks, T.; Silva, A. J.: Mechanism for the learning deficits in a mouse model of neurofibromatosis type 1. Nature 415: 526-530, 2002.
van de Vijver, M. J.; Peterse, J. L.; Mooi, W. J.; Wisman, P.; Lomans, J.; Dalesio, O.; Nusse, R.: NEU-protein overexpression in breast cancer: association with comedo-type ductal carcinoma in situ and limited prognostic value in stage II breast cancer. New Eng. J. Med. 319: 1239-1245, 1988.
Xie, D.; Shu, X. O.; Deng, Z.; Wen, W.-Q.; Creek, K. E.; Dai, Q., Gao, Y.-T.; Jin, F.; Zheng, W.: Population-based, case-control study of HER2 genetic polymorphism and breast cancer risk. J. Nat. Cancer Inst. 92: 412-417, 2000.
Yamamoto, T.; Ikawa, S.; Akiyama, T.; Semba, K.; Nomura, N.; Miyajima, N.; Saito, T.; Toyoshima, K.: Similarity of protein encoded by the human c-erb-B-2 gene to epidermal growth factor receptor. Nature 319:230-234, 1986.
Yang-Feng, T. L.; Schechter, A. L.; Weinberg, R. A.; Francke, U.: Oncogene from rat neuro/glioblastomas (human gene symbol NGL) is located on the proximal long arm of human chromosome 17 and EGFR is confirmed at 7p13-q11.2. (Abstract) Cytogenet. Cell Genet. 40:784 only, 1985.
Yu, D.; Jing, T.; Liu, B.; Yao, J.; Tan, M.; McDonnell, T. J.; Hung, M.-C.: Overexpression of ErbB2 blocks Taxol-induced apoptosis by up-regulation of p21Cip1, which inhibits p34Cdc2 kinase. Molec. Cell 2: 581-591, 1998.
Fukuhara, S.; Rowley, J. D.; Variakojis, D.; Sweet, D. L.: Chromosome abnormalities in poorly differentiated lymphocytic lymphoma. Cancer Res. 39: 3119-3128, 1979.
Ohno, H.; Fukuhara, S.; Takahashi, R.; Mihara, K.; Sugiyama, T.; Doi, S.; Uchino, H.; Toyoshima, K.: c-yes and bcl-2 genes located on 18q21.3 in a follicular lymphoma cell line carrying a t(14;18) chromosomal translocation. Int. J. Cancer 39: 785-788, 1987
Semba, K.; Nishizawa, M.; Satoh, H.; Fukushige, S.; Yoshida, M. C.; Sasaki, M.; Matsubara, K.; Yamamoto, T.; Toyoshima, K.: Nucleotide sequence and chromosomal mapping of the human c-yes-2 gene. Jpn. J. Cancer Res. 79: 710-717, 1988.
Semba, K.; Yamanashi, Y.; Nishizawa, M.; Sukegawa, J.; Yoshida, M.; Sasaki, M.; Yamamoto, T.; Toyoshima, K.: Location of the c-yes gene on the human chromosome and its expression in various tissues. Science 227:1038-1040, 1985.
Silverman, G. A.; Kuo, W.-L.; Taillon-Miller, P.; Gray, J. W.: Chromosomal reassignment: YACs containing both YES1 and thymidylate synthase map to the short arm of chromosome 18. Genomics 15: 442-445, 1993.
Sukegawa, J.; Semba, K.; Yamanashi, Y.; Nishizawa, M.; Miyajima, N.; Yamamoto, T.; Toyoshima, K.: Characterization of cDNA clones for the human c-yes gene. Molec. Cell. Biol. 7: 41-47, 1987.
Monteiro, R. C.; Hostoffer, R. W.; Cooper, M. D.; Bonner, J. R.; Gartland, G. L.; Kubagawa, H.: Definition of immunoglobulin A receptors on eosinophils and their enhanced expression in allergic individuals. J. Clin. Invest. 92: 1681-1685, 1993
Narita, I.; Goto, S.; Saito, N.; Sakatsume, M.; Jin, S.; Omori, K.; Gejyo, F.: Genetic polymorphisms in the promoter and 5-prime UTR region of the Fc alpha receptor (CD89) are not associated with a risk of IgA nephropathy. J. Hum. Genet. 46: 694-698, 2001.
Pleass, R. J.; Andrews, P. D.; Kerr, M. A.; Woof, J. M.: Alternative splicing of the human IgA Fc receptor CD89 in neutrophils and eosinophils. Biochem. J. 318: 771-777, 1996.
de Wit, T. P. M.; Morton, H. C.; Capel, P. J. A.; van de Winkel, J. G. J.: Structure of the gene for the human myeloid IgA Fc receptor (CD89). J. Immun. 155: 1203-1209, 1995.
Kremer, E. J.; Kalatzis, V.; Baker, E.; Callen, D. F.; Sutherland, G. R.; Maliszewski, C. R.: The gene for the human IgA Fc receptor maps to 19q13.4. Hum. Genet. 89: 107-108, 1992.
Maliszewski, C. R.; March, C. J.; Schoenborn, M. A.; Gimpel, S.; Shen, L.: Expression cloning of a human Fc receptor for IgA. J. Exp. Med. 172: 1665-1672, 1990.
Shimokawa, T.; Tsuge, T.; Okumura, K.; Ra, C.: Identification and characterization of the promoter for the gene encoding the human myeloid IgA Fc receptor (Fc-alpha-R, CD89). Immunogenetics 51: 945-954, 2000
Tsuge, T.; Shimokawa, T.; Horikoshi, S.; Tomino, Y.; Ra, C.: Polymorphism in promoter region of Fc-alpha receptor gene in patients with Ig Anephropathy. Hum. Genet. 108: 128-133, 2001.
Dillon, J. S.; Tanizawa, Y.; Wheeler, M. B.; Leng, X.-H.; Ligon, B. B.; Rabin, D. U.; Yoo-Warren, H.; Permutt, M. A.; Boyd, A. E., III: Cloning and functional expression of the human glucagon-like peptide-1 (GLP-1) receptor. Endocrinology 133: 1907-1910, 1993.
Kershaw, E. E.; Chua, S. C., Jr.; Leibel, R. L.: Localization of a (CA)n repeat in glucagon-like peptide-1 receptor gene (Glp1r) to proximal mouse chromosome 17 and its linkage to other markers. Mammalian Genome 6: 301-303, 1995.
Stoffel, M.; Espinosa, R., III; Le Beau, M. M.; Bell, G. I.: Human glucagon-like peptide-1 receptor gene: localization to chromosome band 6p21 by fluorescence in situ hybridization and linkage of a highly polymorphic simple tandem repeat DNA polymorphism to other markers on chromosome 6. Diabetes 42: 1215-1218, 1993.
Thorens, B.: Expression cloning of the pancreatic beta cell receptor for the gluco-incretin hormone glucagon-like peptide 1. Proc. Nat. Acad. Sci. 89: 8641-8645, 1992.
Cohen, P.; Miyazaki, M.; Socci, N. D.; Hagge-Greenberg, A. Liedtke, W.; Soukas, A. A.; Sharma, R.; Hudgins, L. C.; Ntambi, J. M.; Friedman, J. M.: Role for stearoyl-CoA desaturase-1 in leptin-mediated weightloss. Science 297: 240-243, 2002.
Blangy, A.; Lane, H. A.; d'Herin, P.; Harper, M.; Kress, M.; Nigg, E. A.: Phosphorylation by p34(cdc2) regulates spindle association of human Eg5, a kinesin-related motor essential for bipolar spindle formation in vivo. Cell 83: 1159-1169, 1995.
Stewart, R. J.; Pesavento, P. A.; Woerpel, D. N.; Goldstein, L. S. B.: Identification and partial characterization of six members of the kinesin super family in Drosophila. Proc. Nat. Acad. Sci. 88:8470-8474, 1991.
Tihy, F.; Kress, M.; Harper, M.; Dutrillaux, B.; Lemieux, N.: Localization of the human kinesin-related gene to band 10q24 by fluorescence in situ hybridization. Genomics 13: 1371-1372, 1992.
Tassabehji, M.; Newton, V. E.; Liu, X.-Z.; Brady, A.; Donnai, D.; Krajewska-Walasek, M.; Murday, V.; Norman, A.; Obersztyn, E.; Reardon, W.; Rice, J. C.; Trembath, R.; Wieacker, P.; Whiteford, M.; Winter, R.; Read, A. P.: The mutational spectrum in Waardenburg syndrome. Hum. Molec. Genet. 4: 2131-2137, 1995
Cetta, F.; Chiappetta, G.; Melillo, R. M.; Petracci, M.; Montalto, G.; Santoro, M.; Fusco, A.: The ret/ptc1 oncogene is activated in familial adenomatous polyposis-associated thyroid papillary carcinomas. J. Clin. Endocr. Metab. 83: 1003-1006, 1998.
Decker, R. A.; Peacock, M. L.; Watson, P.: Hirschsprung disease in MEN 2A: increased spectrum of RET exon 10 genotypes and strong genotype-phenotype correlation. Hum. Molec. Genet. 7: 129-134, 1998.
Donis-Keller, H.; Dou, S.; Chi, D.; Carlson, K. M.; Toshima, K.; Lairmore, T. C.; Howe, J. R.; Moley, J. F.; Goodfellow, P.; Wells, S. A., Jr.: Mutations in the RET proto-oncogene are associated with MEN 2A and FMTC. Hum. Molec. Genet. 2: 851-856, 1993.
Doray, B.; Salomon, R.; Amiel, J.; Pelet, A.; Touraine, R.; Billaud, M.; Attie, T.; Bachy, B.; Munnich, A.; Lyonnet, S.: Mutation of the RET ligand, neurturin, supports multigenic inheritance in Hirschsprung disease. Hum. Molec. Genet. 7: 1449-1452, 1998.
Edery, P.; Lyonnet, S.; Mulligan, L. M.; Pelet, A.; Dow, E.; Abel, L.; Holder, S.; Nihoul-Fekete, C.; Ponder, B. A. J.; Munnich, A.: Mutations of the RET proto-oncogene in Hirschsprung's disease. Nature 367:378-380, 1994
Eng, C.: The RET proto-oncogene in multiple endocrine neoplasia type 2 and Hirschsprung's disease. New Eng. J. Med. 335: 943-951, 1996.
Eng, C.; Crossey, P. A.; Mulligan, L. M.; Healey, C. S.; Houghton, C.; Prowse, A.; Chew, S. L.; Dahia, P. L. M.; O'Riordan, J. L. H.; Toledo, S. P. A.; Smith, D. P.; Maher, E. R.; Ponder, B. A. J.: Mutations in the RET proto-oncogene and the von Hippel-Lindau disease tumour suppressor gene in sporadic and syndromic phaeochromocytomas. J. Clin. Genet. 32: 934-937, 1995.
Eng, C.; Mulligan, L. M.: Mutations of the RET proto-oncogene in the multiple endocrine neoplasia type 2 syndromes, related sporadic tumours, and Hirschsprung disease. Hum. Mutat. 9: 97-109, 1997.
Eng, C.; Mulligan, L. M.; Smith, D. P.; Healey, C. S.; Frilling, A.; Raue, F.; Neumann, H. P. H.; Pfragner, R.; Behmel, A.; Lorenzo, M. J.; Stonehouse, T. J.; Ponder, M. A.; Ponder, B. A. J.: Mutation of the RET protooncogene in sporadic medullary thyroid carcinoma. Genes Chromosomes Cancer 12: 209-212, 1995.
Eng, C.; Smith, D. P.; Mulligan, L. M.; Healey, C. S.; Zvelebil, M. J.; Stonehouse, T. J.; Ponder, M. A.; Jackson, C. E.; Waterfield, M. D.; Ponder, B. A. J.: A novel point mutation in the tyrosine kinase domain of the RET protooncogene in sporadic medullary thyroid carcinoma and in a family with FMTC. Oncogene 10: 509-513, 1995.
Eng, C.; Smith, D. P.; Mulligan, L. M.; Nagai, M. A.; Healey, C. S.; Ponder, M. A.; Gardner, E.; Scheumann, G. F. W.; Jackson, C. E.; Tunnacliffe, A.; Ponder, B. A. J.: Point mutation within the tyrosine kinase domain of the RET proto-oncogene in multiple endocrine neoplasia type 2B and related sporadic tumors. Hum. Molec. Genet. 3:237-241, 1994.
Fearon, E. R.: Human cancer syndromes: clues to the origin and nature of cancer. Science 278: 1043-1050, 1997.
Fitze, G.; Schreiber, M.; Kuhlisch, E.; Schackert, H. K.; Roesner, D.: Association of RET protooncogene codon 45 polymorphism with Hirschsprung disease. (Letter) Am. J. Hum. Genet. 65: 1469-1473, 1999.
Gardner, E.; Mulligan, L. M.; Eng, C.; Healey, C. S.; Kwok, J. B. J.; Ponder, M. A.; Ponder, B. A. J.: Haplotype analysis of MEN2 mutations. Hum. Molec. Genet. 3: 1771-1774, 1994.
Grieco, M.; Santoro, M.; Berlingieri, M. T.; Melillo, R. M.; Donghi, R.; Bongarzone, I.; Pierotti, M. A.; Della Porta, G.; Fusco, A.; Vecchio, G.: PTC is a novel rearranged form of the ret proto-oncogene and is frequently detected in vivo in human thyroid papillary carcinomas. Cell 60:557-563, 1990.
Hofstra, R. M. W.; Landsvater, R. M.; Ceccherini, I.; Stulp, R. P.; Stelwagen, T.; Luo, Y.; Pasini, B.; Hoppener, J. W. M.; Ploosvan Amstel, H. K.; Romeo, G.; Lips, C. J. M.; Buys, C. H. C. M.: A mutation in the RET proto-oncogene associated with multiple endocrine neoplasia type 2B and sporadic medullary thyroid carcinoma. Nature 367:375-376, 1994.
Hoppener, J. W. M.; Lips, C. J. M.: RET receptor tyrosine kinase gene mutations: molecular biological, physiological and clinical aspects. Europ. J. Clin. Invest. 26: 613-624, 1996.
Hoppner, W.; Ritter, M. M.: A duplication of 12 bp in the critical cysteine rich domain of the RET proto-oncogene results in a distinct phenotype of multiple endocrine neoplasia type 2A. Hum. Molec. Genet. 6:587-590, 1997.
Ikeda, I.; Ishizaka, Y.; Tahira, T.; Suzuki, T.; Onda, M.; Sugimura, T.; Nagao, M.: Specific expression of the ret proto-oncogene in human neuroblastoma cell lines. Oncogene 5: 1291-1296, 1990.
Ishizaka, Y.; Itoh, F.; Tahira, T.; Ikeda, I.; Sugimura, T.; Tucker, J.; Fertitta, A.; Carrano, A. V.; Nagao, M.: Human ret proto-oncogene mapped to chromosome 10q11.2. Oncogene 4: 1519-1521, 1989.
Japon, M. A.; Urbano, A. G.; Saez, C.; Segura, D. I.; Cerro, A. L.; Dieguez, C.; Alvarez, C. V.: Glial-derived neurotropic factor and RET gene expression in normal human anterior pituitary cell types and in pituitary tumors. J. Clin. Endocr. Metab. 87: 1879-1884, 2002.
Julies, M. G.; Moore, S. W.; Kotze, M. J.; du Plessis, L.: Novel RET mutations in Hirschsprung's disease patients from the diverse South African population. Europ. J. Hum. Genet. 9: 419-423, 2001.
Klugbauer, S.; Demidchik, E. P.; Lengfelder, E.; Rabes, H. M.: Detection of a novel type of RET rearrangement (PTC5) in thyroid carcinomas after Chernobyl and analysis of the involved RET-fused gene RFG5. Cancer Res. 58: 198-203, 1998.
Amagai, M.; Nishikawa, T.; Nousari, H. C.; Anhalt, G. J.; Hashimoto, T.: Antibodies against desmoglein 3 (pemphigus vulgaris antigen) are present in sera from patients with paraneoplastic pemphigus and cause acantholysis in vivo in neonatal mice. J. Clin. Invest. 102:775-782, 1998.
Arnemann, J.; Spurr, N. K.; Buxton, R. S.: The human gene (DSG3) coding for the pemphigus vulgaris antigen is, like the genes coding for the other two known desmogleins, assigned to chromosome 18. Hum. Genet. 89: 347-350, 1992.
Ishikawa, H.; Silos, S. A.; Tamai, K.; Copeland, N. G.; Gilbert, D. J.; Jenkins, N. A.; Uitto, J.: cDNA cloning and chromosomal assignment of the mouse gene for desmoglein 3 (Dsg3), the pemphigus vulgaris antigen. Mammalian Genome 5: 803-804, 1994.
Silos, S. A.; Tamai, K.; Li, K.; Kivirikko, S.; Kouba, D.; Christiano, A. M.; Uitto, J.: Cloning of the gene for human pemphigus vulgaris antigen (desmoglein 3), a desmosomal cadherin. J. Biol. Chem. 271:17504-17511, 1996.
Hamajima, N.; Matsuda, K.; Sakata, S.; Tamaki, N.; Sasaki, M.; Nonaka, M.: A novel gene family defined by human dihydropyrimidinase and three related proteins with differential tissue distribution. Gene 180:157-163, 1996.
Sharp, D.; Blinderman, L.; Combs, K. A.; Kienzle, B.; Ricci, B.; Wager-Smith, K.; Gil, C. M.; Turck, C. W.; Bouma, M.-E.; Rader, D. J.; Aggerbeck, L. P.; Gregg, R. E.; Gordon, D. A.; Wetterau, J. R.: Cloning and gene defects in microsomal triglyceride transfer protein associated with abetalipoproteinaemia. Nature 365: 65-69, 1993
Julier, C.; Lathrop, M.; Lalouel, J. M.; Kaplan, J. C.: Use of-multilocus tests of gene order: example for chromosome 22. (Abstract) Cytogenet. Cell Genet. 40: 663-664, 1985.
Julier, C.; Lathrop, M.; Lalouel, J. M.; Reghis, A.; Szajnert, M. F.; Kaplan, J. C.: New restriction fragment length polymorphisms on human chromosome 22 at loci SIS, MB and IGLV. (Abstract) Cytogenet. Cell Genet. 40: 664 only, 1985.
Julier, C.; Reghis, A.; Szajnert, M. F.; Kaplan, J. C.; Lathrop, G. M.; Lalouel, J. M.: A preliminary linkage map of human chromosome 22. (Abstract) Cytogenet. Cell Genet. 40: 665 only, 1985.
Bodmer, J. G.; Marsh, S. G. E.; Albert, E.: Nomenclature for factors of the HLA system, 1989. Immun. Today 11: 3-10, 1990.
Fitzgerald, L. A.; Poncz, M.; Steiner, B.; Rall, S. C., Jr.; Bennett, J. S.; Phillips, D. R.: Comparison of cDNA-derived protein sequences of the human fibronectin and vitronectin receptor alpha-subunits and platelet glyco protein IIb. Biochemistry 26: 8158-8165, 1987.
Sosnoski, D.; Emanuel, B. S.; Hawkins, A. L.; van Tuinen, P.; Ledbetter, D. H.; Nussbaum, R. L.; Kaos, F.-T.; Schwartz, E.; Phillips, D.; Bennett, J. S.; Fitzgerald, L. A.;
Poncz, M.: Chromosomal localization of the genes for the vitronectin and fibronectin receptors alpha-subunits and for platelet glycoproteins IIb and IIIa. J. Clin. Invest. 81:1993-1998, 1988.
Spurr, N. K.; Rooke, L.: Confirmation of the assignment of the vitronectin (VNRA) and fibronectin (FNRA) receptor alpha-subunits. Ann. Hum. Genet. 55: 217-223, 1991.
Akula, S. M.; Pramod, N. P.; Wang, F.-Z.; Chandran, B.: Integrin alpha-3/beta-1 (CD 49c/29) is a cellular receptor for Kaposi's sarcoma-associated herpesvirus (KSHV/HHV-8) entry into the target cells. Cell 108:407-419, 2002.
Arregui, C.; Pathre, P.; Lilien, J.; Balsamo, J.: The nonreceptor tyrosine kinase Fer mediates cross-talk between N-cadherin and beta-1-integrins. J. Cell Biol. 149: 1263-1273, 2000.
Giuffra, L. A.; Lichter, P.; Wu, J.; Kennedy, J. L.; Pakstis, A. J.; Rogers, J.; Kidd, J. R.; Harley, H.; Jenkins, T.; Ward, D. C.; Kidd, K. K.: Genetic and physical mapping and population studies of a fibronectin receptor beta-subunit-like sequence on human chromosome 19. Genomics 8: 340-346, 1990.
Giuffra, L. A.; Wu, J.; Lichter, P.; Kennedy, J. L.; Castiglione, C.; Pakstis, A. J.; Ward, D.; Kidd, K. K.: Mapping of a fibronectin receptor beta subunit-like sequence to chromosome 19. (Abstract) Am. J. Hum. Genet. 45 (suppl.): A141 only, 1989.
Goodfellow, P. J.; Nevanlinna, H. A.; Gorman, P.; Sheer, D.; Lam, G.; Goodfellow, P. N.: Assignment of the gene encoding the beta-subunit of the human fibronectin receptor (beta-FNR) to chromosome 10p11.2. Ann. Hum. Genet. 53: 15-22, 1989.
Graus-Porta, D.; Blaess, S.; Senften, M.; Littlewood-Evans, A.; Damsky, C.; Huang, Z.; Orban, P.; Klein, R.; Schittny, J. C.; Muller, U.: Beta-1-class integrins regulate the development of laminae and folia in the cerebral and cerebellar cortex. Neuron 31: 367-379, 2001.
Hynes, R. O.: Integrins: a family of cell surface receptors. Cell 48:549-554, 1987.
Johansson, S.; Forsberg, E.; Lundgren, B.: Comparison of fibronectin receptors from rat hepatocytes and fibroblasts. J. Biol. Chem. 262:7819-7824, 1987.
Lu, T. T.; Cyster, J. G.: Integrin-mediated long-term B cell retention in the splenic marginal zone. Science 297: 409-412, 2002.
Messer Peters, P.; Kamarck, M. E.; Hemler, M. E.; Strominger, J. L.; Ruddle, F. H.: Genetic and biochemical characterization of human lymphocyte cell surface antigens: the A-1A5 and A-3A4 determinants. J. Exp. Med. 159: 1441-1454, 1984.
Pytela, R.; Pierschbacher, M. D.; Ginsberg, M. H.; Plow, E. F.; Ruoslahti, E.: Platelet membrane glyco protein IIb/IIIa: member of a family of arg-gly-asp-specific adhesion receptors. Science 231:1159-1162, 1986.
Woods, V. L., Jr.; Pischel, K. D.; Avery, E. D.; Bluestein, H. G.: Antigenic polymorphism of human very late activation protein-2(platelet glyco protein Ia-IIa): platelet alloantigen Hc(a). J. Clin. Invest. 83: 978-985, 1989.
Wu, J. S.; Giuffra, L. A.; Goodfellow, P. J.; Myers, S.; Carson, N. L.; Anderson, L.; Hoyle, L. S.; Simpson, N. E.; Kidd, K. K.: The beta subunit locus of the human fibronectin receptor: DNA restriction fragment length polymorphism and linkage mapping studies. Hum. Genet. 83:383-390, 1989.
Wildhage, I.; Trusheim, H.; Goke, B.; Lankat-Buttgereit, B.: Gene expression of the human glucagon-like peptide-1 receptor is regulated by Sp1 and Sp3. Endocrinology 140: 624-631, 1999.
Chambers, S. M.; Morris, B. J.: Glucagon receptor gene mutationin essential hypertension. (Letter) Nature Genet. 12: 122, 1996.
Hager, J.; Hansen, L.; Vaisse, C.; Vionnet, N.; Philippi, A.; Poller, W.; Velho, G.; Carcassi, C.; Contu, L.; Julier, C.; Cambien, F.; Passa, P.; Lathrop, M.; Kindsvogel, W.; Demenais, F.; Nishimura, E.; Froguel, P.: A missense mutation in the glucagon receptor gene is associated with non-insulin-dependent diabetes mellitus. Nature Genet. 9: 299-304, 1995.
Lok, S.; Kuijper, J. L.; Jelinek, L. J.; Kramer, J. M.; Whitmore, T. E.; Sprecher, C. A.; Mathewes, S.; Grant, F. J.; Biggs, S. H.; Rosenberg, G. B.; Sheppard, P. O.; O'Hara, P. J.; Foster, D. C.; Kindsvogel, W.: The human glucagon receptor encoding gene: structure, cDNA sequence and chromosomal localization. Gene 140: 203-209, 1994.
Menzel, S.; Stoffel, M.; Espinosa, R., III; Fernald, A. A.; LeBeau, M. M.; Bell, G. I.: Localization of the glucagon receptor geneto human chromosome band 17q25. Genomics 20: 327-328, 1994.
Bae, J.; Leo, C. P.; Hsu, S. Y.; Hsuch, A. J. W.: MCL-1S, a splicing variant of the antiapoptotic BCL-2 family member MCL-1, encodes aproapoptotic protein possessing only the BH3 domain. J. Biol. Chem. 275:25255-25261, 2000.
Craig, R. W.; Jabs, E. W.; Zhou, P.; Kozopas, K. M.; Hawkins, A. L.; Rochelle, J. M.; Seldin, M. F.; Griffin, C. A.: Human and mouse chromosomal mapping of the myeloid cell leukemia-1 gene: MCL1 maps to human chromosome 1q21, a region that is frequently altered in preneoplastic and neoplastic disease. Genomics 23: 457-463, 1994.
Kozopas, K. M.; Yang, T.; Buchan, H. L.; Zhou, P.; Craig, R. W.: MCL1, a gene expressed in programmed myeloid cell differentiation, has sequence similarity to BCL2. Proc. Nat. Acad. Sci. 90: 3516-3520, 1993.
Rinkenberger, J. L.; Horning, S.; Klocke, B.; Roth, K.; Korsmeyer, S. J.: Mcl-1 deficiency results in peri-implantation embryonic lethality. Genes Dev. 14: 23-27, 2000.
Pellegata, N. S.; Dieguez-Lucena, J. L.; Joensuu, T.; Lau, S.; Montgomery, K. T.; Krahe, R.; Kivela, T.; Kucherlapati, R.; Forsius, H.; de la Chapelle, A.: Mutations in KERA, encoding keratocan, cause cornea plana. Nature Genet. 25: 91-95, 2000.
Lehmann, J. M.; McKee, D. D.; Watson, M. A.; Willson, T. M.; Moore, J. T.; Kliewer, S. A.: The human orphan nuclear receptor PXR is activated by compounds that regulate CYP3A4 gene expression and cause drug interactions. J. Clin. Invest. 102: 1016-1023, 1998.
Aberdam, E.; Bertolotto, C.; Sviderskaya, E. V.; de Thillot, V.; Hemesath, T. J.; Fisher, D. E.; Bennett, D. C.; Ortonne, J.-P.; Ballotti, R.: Involvement of microphthalmia in the inhibition of melanocyte lineage differentiation and of melanogenesis by agouti signal protein. J. Biol. Chem. 273: 19560-19565, 1998.
Bondur and, N.; Pingault, V.; Goerich, D. E.; Lemort, N.; Sock, E.; Le Caignec, C.; Wegner, M.; Goossens, M.: Interaction among SOX10, PAX3 and MITF, three genes altered in Waardenburg syndrome. Hum. Molec. Genet. 9: 1907-1917, 2000
Jackson, I. J.; Raymond, S.: Manifestations of microphthalmia. Nature Genet. 8: 209-210, 1994.
Lalwani, A. K.; Attaie, A.; Randolph, F. T.; Deshmukh, D.; Wang, C.; Mhatre, A.; Wilcox, E.: Point mutation in the MITF gene causing Waardenburg syndrome type II in a three-generation Indian family. Am. J. Med. Genet. 80: 406-409, 1998.
Moore, K. J.: Insight into the microphthalmia gene. Trends Genet. 11:442-448, 1995.
Minoshima, S.; Fukuyama, R.; Yamamoto, T.; Shimizu, N.: Mapping of human long-chain acyl-CoA synthetase to chromosome 4. (Abstract) Cytogenet. Cell Genet. 58: 1888 only, 1991.
Cantu, E. S.; Sprinkle, T. J.; Ghosh, B.; Singh, I.: The human palmitoyl-CoA ligase (FACL2) gene maps to the chromosome 4q34-q35 region by fluorescence in situ hybridization (FISH) and somatic cell hybrid panels. Genomics 28: 600-602, 1995.
Rountree, M. R.; Bachman, K. E.; Baylin, S. B.: DNMT1 binds HDAC2 and a new co-repressor, DMAP1, to form a complex at replication foci. Nature Genet. 25: 269-277, 2000.
Pyronnet, S.; Imataka, H.; Gingras, A.-C.; Fukunaga, R.; Hunter, T.; Sonenberg, N.: Human eukaryotic translation initiation factor 4G (eIF4G) recruits Mnk1 to phosphorylate eIF4E. EMBO J. 18: 270-279, 1999.
Waskiewicz, A. J.; Flynn, A.; Proud, C. G.; Cooper, J. A.: Mitogen-activated protein kinases activate the serine/threonine kinases Mnk1 and Mnk2. EMBO J. 16: 1909-1920, 1997.
Zhang, Y.; Saison, M.; Spaepen, M.; De Strooper, B.; Van Leuven, F.; David, G.; Van den Berghe, H.; Cassiman, J.-J.: Mapping of human fibronectin receptor beta subunit gene to chromosome 10. Somat. Cell Molec. Genet. 14: 99-104, 1988.
Schoenmakers, E. P. P. M.; Wanschura, S.; Mols, R.; Bullerdiek, J.; Van den Berghe, H.; Van de Ven, W. J. M.: Recurrent rearrangements in the high mobility group protein gene, HMGI-C, in benign mesenchymal tumours. Nature Genet. 10: 436-444, 1995.
Beck, Y.; Oren, R.; Amit, B.; Levanon, A.; Gorecki, M.; Hartman, J. R.: Human Mn superoxide dismutase cDNA sequence. Nucleic Acids Res. 15: 9076, 1987.
Church, S. L.; Grant, J. W.; Meese, E. U.; Trent, J. M.: Sublocalization of the gene encoding manganese superoxide dismutase (MnSOD/SOD2) to 6q25 by fluorescence in situ hybridization and somatic cell hybrid mapping. Genomics 14: 823-825, 1992.
Creagan, R.; Tischfield, J.; Ricciuti, F.; Ruddle, F. H.: Chromosome assignments of genes in man using mouse-human somatic cell hybrids: mitochondrial superoxide dismutase (indophenol oxidase-B, tetrameric) to chromosome 6. Human genetik 20: 203-209, 1973.
Figueroa, F.; Vincek, V.; Kasahara, M.; Bell, G. I.; Klein, J.: Mapping of the Sod-2 locus into the t complex on mouse chromosome 17. Immunogenetics 28: 260-264, 1988.
Heckl, K.: Isolation of cDNAs encoding human manganese superoxide dismutase. Nucleic Acids Res. 16: 6224, 1988.
Li, Y.; Huang, T.-T.; Carlson, E. J.; Melov, S.; Ursell, P. C.; Olson, J. L.; Noble, L. J.; Yoshimura, M. P.; Berger, C.; Chan, P. H.; Wallace, D. C.; Epstein, C. J.: Dilated cardiomyopathy and neonatal lethality in mutant mice lacking manganese superoxide dismutase. Nature Genet. 11: 376-381, 1995.
Melov, S.; Coskun, P.; Patel, M.; Tuinstra, R.; Cottrell, B.; Jun, A. S.; Zastawny, T. H.; Dizdaroglu, M.; Goodman, S. I.; Huang, T.-T.; Miziorko, H.; Epstein, C. J.; Wallace, D. C.: Mitochondrial disease in superoxide dismutase 2 mutant mice. Proc. Nat. Acad. Sci. 96:846-851, 1999
Melov, S.; Schneider, J. A.; Day, B. J.; Hinerfeld, D.; Coskun, P.; Mirra, S. S.; Crapo, J. D.; Wallace, D. C.: A novel neurological phenotype in mice lacking mitochondrial manganese superoxide dismutase. Nature Genet. 18: 159-163, 1998.
Michelson, A. M.; McCord, J. M.; Fridovich, I.: Superoxide and Superoxide Dismutases. New York: Academic Press, 1977.
Pauling, L.: The discovery of the superoxide radical. Trends Biochem. Sci. 4(11): 270-271, 1979.
Rosenblum, J. S.; Gilula, N. B.; Lerner, R. A.: On signal sequence polymorphisms and diseases of distribution. Proc. Nat. Acad. Sci. 93:4471-4473, 1996.
Yoshimitsu, K.; Nichi, Y.; Kobayashi, Y.; Yoshimura, O.; Ohama, K.; Oguma, N.; Usui, T.: Decreased superoxide dismutase-2 activity in a patient with ring chromosome 6. Am. J. Med. Genet. 28: 211-214, 1987.
Klugbauer, S.; Rabes, H. M.: The transcription coactivator HTIF1 and a related protein are fused to the RET receptor tyrosine kinase in childhood papillary thyroid carcinomas. Oncogene 18: 4388-4393, 1999.
Lairmore, T. C.; Dou, S.; Howe, J. R.; Chi, D.; Carlson, K.; Veile, R.; Mishra, S. K.; Wells, S. A., Jr.; Donis-Keller, H.: A 1.5-megabase yeast artificial chromosome contig from human chromosome 10q11.2 connecting three genetic loci (RET, D10S94, and D10S102) closely linked to the MEN2A locus. Proc. Nat. Acad. Sci. 90: 492-496, 1993.
Lombardo, F.; Baudin, E.; Chiefari, E.; Arturi, F.; Bardet, S.; Caillou, B.; Conte, C.; Dallapiccola, B.; Giuffrida, D.; Bidart, J.-M.; Schlumberger, M.; Filetti, S.: Familial medullary thyroid carcinoma: clinical variability and low aggressiveness associated with RET mutation at codon 804. J. Clin. Endocr. Metab. 87: 1674-1680, 2002.
Lore, F.; Di Cairano, G.; Talidis, F.: Unilateral renal agenesis in a family with medullary thyroid carcinoma. (Letter) New Eng. J. Med. 342: 1218-1219, 2000.
Machens, A.; Gimm, O.; Hinze, R.; Hoppner, W.; Boehm, B. O.; Dralle, H.: Genotype-phenotype correlations in hereditary medullary thyroid carcinoma: oncological features and biochemical properties. J. Clin. Endocr. Metab. 86: 1104-1109, 2001.
Mendelsohn, C.; et al; et al: Function of the retinoic acid receptors (RARs) during development (II). Multiple abnormalities at various stages of organogenesis in RAR double mutants. Development 120:2749-2771, 1994.
Menko, F. H.; van der Luijt, R. B.; de Valk, I. A. J.; Toorians, A. W. F. T.; Sepers, J. M.; van Diest, P. J.; Lips, C. J. M.: A typical MEN type 2B associated with two germline RET mutations on the same allele not involving codon 918. J. Clin. Endocr. Metab. 87: 393-397, 2002.
Mulligan, L. M.; Kwok, J. B. J.; Healey, C. S.; Elsdon, M. J.; Eng, C.; Gardner, E.; Love, D. R.; Mole, S. E.; Moore, J. K.; Papi, L.; Ponder, M. A.; Telenius, H.; Tunnacliffe, A.; Ponder, B. A. J.: Germ-line mutations of the RET protooncogene in multiple endocrine neoplasia type 2A. Nature 363: 458-460, 1993.
Munnes, M.; Fanaei, S.; Schmitz, B.; Muiznieks, I.; Holschneider, A. M.; Doerfler, W.: Familial form of Hirschsprung disease: nucleotide sequence studies reveal point mutations in the RET proto-oncogene in two of six families but not in other candidate genes. Am. J. Med. Genet. 94: 19-27, 2000.
Nakata, T.; Kitamura, Y.; Shimizu, K.; Tanaka, S.; Fujimori, M.; Yokoyama, S.; Ito, K.; Emi, M.: Fusion of a novel gene, ELKS, to RET due to translocation t(10;12)(q11;p13) in a papillary thyroid carcinoma. Genes Chromosomes Cancer 25: 97-103, 1999.
Niccoli-Sire, P.; Murat, A.; Rohmer, V.; Franc, S.; Chabrier, G.; Baldet, L.; Maes, B.; Savagner, F.; Giraud, S.; Bezieau, S.; Kottler, M.-L.; Morange, S.; Conte-Devolx, B.: The French Calcitonin Tumors Study Group (GETC).: Familial medullary thyroid carcinoma with noncysteine RET mutations: phenotype-genotype relationship in a large series of patients. J. Clin. Endocr. Metab. 86: 3746-3753, 2001.
Pachnis, V.; Mankoo, B.; Costantini, F.: Expression of the c-ret proto-oncogene during mouse embryogenesis. Development 119: 1005-1017, 1993.
Pasini, B.; Hofstra, R. M. W.; Yin, L.; Bocciardi, R.; Santamaria, G.; Grootscholten, P. M.; Ceccherini, I.; Patrone, G.; Priolo, M.; Buys, C. H. C. M.; Romeo, G.: The physical map of the human RET proto-oncogene. Oncogene 11:1737-1743, 1995.
Pelet, A.; Geneste, O.; Edery, P.; Pasini, A.; Chappuis, S.; Attie, T.; Munnich, A.; Lenoir, G.; Lyonnet, S.; Billaud, M.: Various mechanisms cause RET-mediated signaling defects in Hirschsprung's disease. J. Clin. Invest. 101: 1415-1423, 1998.
Pigny, P.; Bauters, C.; Wemeau, J.-L.; Houcke, M. L.; Crepin, M.; Caron, P.; Giraud, S.; Calender, A.; Buisine, M.-P.; Kerckaert, J.-P.; Porchet, N.: A novel 9-base pair duplication in RET exon 8 in familial medullary thyroid carcinoma. J. Clin. Endocr. Metab. 84:1700-1704, 1999
Pierotti, M. A.; Santoro, M.; Jenkins, R. B.; Sozzi, G.; Bongarzone, I.; Grieco, M.; Monzini, N.; Miozzo, M.; Herrmann, M. A.; Fusco, A.; Hay, I. D.; Della Porta, G.; Vecchio, G.: Characterization of an inversion on the long arm of chromosome 10 juxtaposing D10S170 and RET and creating the oncogenic sequence RET/PTC. Proc. Nat. Acad. Sci. 89: 1616-1620, 1992.
Rodrigues, G. A.; Park, M.: Dimerization mediated through a leucine zipper activates the oncogenic potential of the met receptor tyrosine kinase. Molec. Cell. Biol. 13: 6711-6722, 1993.
Romeo, G.; Ronchetto, P.; Luo, Y.; Barone, V.; Seri, M.; Ceccherini, I.; Pasini, B.; Bocciardi, R.; Lerone, M.; Kaariainen, H.; Martucciello, G.: Point mutations affecting the tyrosine kinase domain of the RET proto-oncogene in Hirschsprung's disease. Nature 367: 377-378, 1994.
Salvatore, D.; Barone, M. V.; Salvatore, G.; Melillo, R. M.; Chiappetta, G.; Mineo, A.; Fenzi, G.; Vecchio, G.; Fusco, A.; Santoro, M.: Tyrosines 1015 and 1062 are in vivo autophosphorylation sites in Ret and Ret-derived oncoproteins. J. Clin. Endocr. Metab. 85: 3898-3907, 2000.
Robinson, M. F.; Cote, G. J.; Nunziata, V.; Brandi, M. L.; Ferrer, J. P.; Martins Bugalho, M. J. G.; Almeida Ruas, M. M.; Chik, C.; Colantuoni, V.; Gagel, R. F.: Mutation of a specific codon of the RET proto-oncogene in the multiple endocrine neoplasia type 2A/cutaneous lichen amyloidosis-syndrome. (Abstract) Fifth International Workshop on Multiple Endocrine Neoplasia, Stockholm, Archipelago, 1994.
Klinghoffer, R. A.; Mueting-Nelsen, P. F.; Faerman, A.; Shani, M.; Soriano, P.: The two PDGF receptors maintain conserved signaling in vivo despite divergent embryological functions. Molec. Cell 7:343-354, 2001.
Kulkarni, S.; Heath, C.; Parker, S.; Chase, A.; Iqbal, S.; Pocock, C. F.; Kaeda, J.; Cwynarski, K.; Goldman, J. M.; Cross, N. C. P.: Fusion of H4/D10S170 to the platelet-derived growth factor receptor beta in BCR-ABL-negative myeloproliferative disorders with a t(5;10)(q33;q21). Cancer Res. 60: 3592-3598, 2000.
Leal, F.; Williams, L. T.; Robbins, K. C.; Aaronson, S. A.: Evidence that the v-sis gene product transforms by interaction with the receptor for platelet-derived growth factor. Science 230: 327-330, 1985.
Matsui, T.; Heidaran, M.; Miki, T.; Popescu, N.; La Rochelle, W.; Kraus, M.; Pierce, J.; Aaronson, S.: Isolation of a novel receptor cDNA establishes the existence of two PDGF receptor genes. Science 243:800-804, 1989.
Spies, T.; Blanck, G.; Bresnahan, M.; Sands, J.; Strominger, J. L.: A new cluster of genes within the human major histocompatibility complex. Science 243: 214-217, 1989.
Pischel, K. D.; Marlin, S. D.; Springer, T. A.; Woods, V. L., Jr.; Bluestein, H. G.: Polymorphism of lymphocyte function-associated antigen-1 demonstrated by a lupus patient's alloantiserum. J. Clin. Invest. 79: 1607-1614, 1987.
Sanchez-Madrid, F.; Nagy, J.; Robbins, E.; Simon, P.; Springer, T. A.: A human leukocyte differentiation antigen family with distinct alpha subunits and a common beta subunit: the lymphocyte function-associated antigen (LFA-1), the C3bi complement receptor (OKM1/Mac-1), and the p150,95 molecule. J. Exp. Med. 158: 1785-1803, 1983.
Inoguchi, K.; Yoshioka, H.; Khaleduzzaman, M.; Ninomiya, Y.: Them RNA for alpha-1(XIX) collagen chain, a new member of FACITs, contains a long unusual 3-prime untranslated region and displays many unique splicing variants. J. Biochem. 117: 137-146, 1995.
Khaleduzzaman, M.; Sumiyoshi, H.; Ueki, Y.; Inoguchi, K.; Ninomiya, Y.; Yoshioka, H.: Structure of the human type XIX collagen (COL19A1) gene, which suggests it has arisen from an ancestor gene of the FACIT family. Genomics 45: 304-312, 1997.
Myers, J. C.; Sun, M. J.; D'Ippolito, J. A.; Jabs, E. W.; Neilson, E. G.; Dion, A. S.: Human cDNA clones transcribed from an unusually high molecular weight RNA encode a new collagen chain. Gene 123:211-217, 1993.
Yoshioka, H.; Zhang, H.; Ramirez, F.; Mattei, M.-G.; Moradi-Ameli, M.; van der Rest, M.; Gordon, M. K.: Synteny between the loci for a novel FACIT-like collagen (D6S228E) and alpha 1(IX) collagen (COL9A1) on 6q12-q14 in humans. Genomics 13: 884-886, 1992.
Gerecke, D. R.; Olson, P. F.; Koch, M.; Knoll, J. H. M.; Taylor, R.; Hudson, D. L.; Champliaud, M.-F.; Olsen, B. R.; Burgeson, R. E.: Complete primary structure of two splice variants of collagen XII, and assignment of alpha-1(XII) collagen (COL12A1), alpha-1(IX) collagen (COL9A1), and alpha-1(XIX) collagen (COL19A1) to human chromosome 6q12-q13. Genomics 41: 236-242, 1997.
Yan, B.; Heus, J.; Lu, N.; Nichols, R. C.; Raben, N.; Plotz, P. H.: Transcriptional regulation of the human acid alpha-glucosidase gene: identification of a repressor element and its transcription factors Hes-1 and YY1. J. Biol. Chem. 276: 1789-1793, 2001
Yan, B.; Raben, N.; Plotz, P. H.: Hes-1, a known transcriptional repressor, acts as a transcriptional activator for the human acid alpha-glucosidase gene in human fibroblast cells. Biochem. Biophys. Res. Commun. 291: 582-587, 2002.
Copeland, N. G.; Gilbert, D. J.; Chretien, M.; Seidah, N. G.; Jenkins, N. A.: Regional localization of three convertases, PC1 (Nec-1), PC2(Nec-2), and furin (Fur), on mouse chromosomes. Genomics 13: 1356-1358, 1992.
Mbikay, M.; Seidah, N. G.; Chretien, M.; Simpson, E. M.: Chromosomal assignment of the genes for pro protein convertases PC4, PC5, and PACE4 in mouse and human. Genomics 26: 123-129, 1995.
Seidah, N. G.; Mattei, M. G.; Gaspar, L.; Benjannet, S.; Mbikay, M.; Chretien, M.: Chromosomal assignments of the genes for neuroendocrine convertase PC1 (NEC1) to human 5q15-21, neuroendocrine convertase PC2 (NEC2) to human 20p11.1-11.2, and furin (mouse 7[D1-E2] region). Genomics 11:103-107, 1991.
Hall, C. R.; Cole, W. G.; Haynes, R.; Hecht, J. T.: Reevaluation of a genetic model for the development of exostosis in hereditary multiple exostosis. Am. J. Med. Genet. 112: 1-5, 2002
Sipila, K.; Aula, P.: Database for the mutations of the Finnish disease heritage. Hum. Mutat. 19: 16-22, 2002.
Lee, H.-K.; Barbarosie, M.; Kameyama, K.; Bear, M. F.; Huganir, R. L.: Regulation of distinct AMPA receptor phosphorylation sites during bidirectional synaptic plasticity. Nature 405: 955-959, 2000.
Mack, V.; Burnashev, N.; Kaiser, K. M. M.; Rozov, A.; Jensen, V.; Hvalby, O.; Seeburg, P. H.; Sakmann, B.; Sprengel, R.: Conditional restoration of hippocampal synaptic potentiation in GluR-A-deficient mice. Science 292: 2501-2504, 2001.
Puckett, C.; Gomez, C. M.; Korenberg, J. R.; Tung, H.; Meier, T. J.; Chen, X. N.; Hood, L.: Molecular cloning and chromosomal localization of one of the human glutamate receptor genes. Proc. Nat. Acad. Sci. 88:7557-7561, 1991.
Shi, S.-H.; Hayashi, Y.; Petralla, R. S.; Zaman, S. H.; Wenthold, R. J.; Svoboda, K.; Malinow, R.: Rapid spine delivery and redistribution of AMPA receptors after synaptic NMDA receptor activation. Science 284:1811-1816, 1999.
Zamanillo, D.; Sprengel, R.; Hvalby, O.; Jensen, V.; Burnashev, N.; Rozov, A.; Kaiser, K. M. M.; Koster, H. J.; Borchardt, T.; Worley, P.; Lubke, J.; Frotscher, M.; Kelly, P. H.; Sommer, B.; Andersen, P.; Seeburg, P. H.; Sakmann, B.: Importance of AMPA receptors for hippocampal synaptic plasticity but not for spatial learning. Science 284:1805-1811, 1999.
Berkovitz, G. D.; Guerami, A.; Brown, T. R.; MacDonald, P. C.; Migeon, C. J.: Familial gynecomastia with increased extraglandular aromatization of plasma carbon (19)-steroids. J. Clin. Invest. 75:1763-1769, 1985.
Hou, J.; Parrish, J.; Ludecke, H.-J.; Sapru, M.; Wang, Y.; Chen, W.; Hill, A.; Siegel-Bartelt, J.; Northrup, H.; Elder, F. F. B.; Chinault, C.; Horsthemke, B.; Wagner, M. J.; Wells, D. E.: A 4-megabase YAC contig that spans the Langer-Giedion syndrome region on human chromosome 8q24.1: use in refining the location of the trichorhinophalangeal-syndrome and multiple exostoses genes (TRPS1 and EXT1). Genomics 29:87-97, 1995.
Ludecke, H.-J.; Wagner, M. J.; Nardmann, J.; La Pillo, B.; Parrish, J. E.; Willems, P. J.; Haan, E. A.; Frydman, M.; Hamers, G. J. H.; Wells, D. E.; Horsthemke, B.: Molecular dissection of a contiguous gene syndrome: localization of the genes involved in the Langer-Giedion syndrome. Hum. Molec. Genet. 4: 31-36, 1995.
Raskind, W. H.; Conrad, E. U., III; Matsushita, M.; Wijsman, E. M.; Wells, D. E.; Chapman, N.; Sandell, L. J.; Wagner, M.; Houck, J.: Evaluation of locus heterogeneity and EXT1 mutations in 34 families with hereditary multiple exostoses. Hum. Mutat. 11: 231-239, 1998.
Usala, A.-L.; Madigan, T.; Burguera, B.; Sinha, M. K.; Caro, J. F.; Cunningham, P.; Powell, J. G.; Butler, P. C.: Treatment of insulin-resistant diabetic ketoacidosis with insulin-like growth factor I in an adolescent with insulin-dependent diabetes. New Eng. J. Med. 327: 853-857, 1992.
Vaessen, N.; Janssen, J. A.; Heutink, P.; Hofman, A.; Lamberts, S. W. J.; Oostra, B. A.; Pols, H. A. P.; van Duijn, C. M.: Association between genetic variation in the gene for insulin-like growth factor-1 and low birthweight. Lancet 359: 1036-1037, 2002.
Van Wyk, J. J.; Svoboda, M. E.; Underwood, L. E.: Evidence from radioligand assays that somatomedin-C and insulin-like growth factor-I are similar to each other and different from other somatomedins. J. Clin. Endocr. Metab. 50: 206-208, 1980.
Ceccherini, I.; Romei, C.; Barone, V.; Pacini, F.; Martino, E.; Loviselli, A.; Pinchera, A.; Romeo, G.: Identification of the cys634-to-tyrmutation of the RET proto-oncogene in a pedigree with multiple endocrine neoplasia type 2A and localized cutaneous lichen amyloidosis. J. Endocr. Invest. 17: 201-204, 1994.
Axton, R.; Hanson, I.; Danes, S.; Sellar, G.; van Heyningen, V.; Prosser, J.: The incidence of PAX6 mutation in patients with simple aniridia: an evaluation of mutation detection in 12 cases. J. Med. Genet. 34: 279-286, 1997.
Beauchamp, G. R.: Anterior segment dysgenesis keratolenticular adhesion and aniridia. J. Pediat. Ophthal. Strabismus 17: 55-58, 1978.
Crolla, J. A.; Cross, I.; Alkey, N.; Wright, M.; Oley, C. A.: FISH studies in a patient with sporadic aniridia and t(7;11)(q31.2;p13). J. Med. Genet. 33: 66-68, 1996.
Fantes, J.; Redeker, B.; Breen, M.; Boyle, S.; Brown, J.; Fletcher, J.; Jones, S.; Bickmore, W.; Fukushima, Y.; Mannens, M.; Danes, S.; van Heyningen, V.; Hanson, I.: Aniridia-associated cytogenetic rearrangements suggest that a position effect may cause the mutant phenotype. Hum. Molec. Genet. 4: 415-422, 1995.
Hiroi, S.; Harada, H.; Nishi, H.; Satoh, M.; Nagai, R.; Kimura, A.: Polymorphisms in the SOD2 and HLA-DRB1 genes are associated with nonfamilial idiopathic dilated cardiomyopathy in Japanese. Biochem. Biophys. Res. Commun. 261: 332-339, 1999
Nishi, H.; Koga, Y.; Koyanagi, T.; Harada, H.; Imaizumi, T.; Toshima, H.; Sasazuki, T.; Kimura, A.: DNA typing of HLA class II genes injapanese patients with dilated cardiomyopathy. J. Molec. Cell. Cardiol. 27:2385-2392, 1995.
Xie, W.; Barwick, J. L.; Downes, M.; Blumberg, B.; Simon, C. M.; Nelson, M. C.; Neuschwander-Tetri, B. A.; Brunt, E. M.; Guzelian, P. S.; Evans, R. M.: Humanized xenobiotic response in mice expressing nuclear receptor SXR. Nature 406: 435-439, 2000.
Grundmann, U.; Nerlich, C.; Rein, T.; Zettlmeissl, G.: Complete cDNA sequence encoding human beta-galactoside alpha-2,6-sialyltransferase. Nucleic Acids Res. 18: 667 only, 1990.
Wang, X.; Vertino, A.; Eddy, R. L.; Byers, M. G.; Jani-Sait, S. N.; Shows, T. B.; Lau, J. T. Y.: Chromosome mapping and organization of the human beta-galactoside alpha-2,6-sialyltransferase gene: differential and cell-type specific usage of upstream exon sequences in B-lymphoblastoid cells. J. Biol. Chem. 268: 4355-4361, 1993.
Brown, D.; Zhu, X. L.; Sly, W. S.: Localization of membrane-associated carbonic anhydrase type IV in kidney epithelial cells. Proc. Nat. Acad. Sci. 87: 7457-7461, 1990
Fleming, R. E.; Crouch, E. C.; Ruzicka, C. A.; Sly, W. S.: Pulmonary carbonic anhydrase IV: developmental regulation and cell-specifice xpression in the capillary endothelium. Am. J. Physiol. 265: L627-L635, 1993.
Fleming, R. E.; Parkkila, S.; Parkkila, A.-K.; Rajaniemi, H.; Waheed, A.; Sly, W. S.: Carbonic anhydrase IV expression in rat and human gastrointestinal tract regional, cellular, and subcellular localization. J. Clin. Invest. 96: 2907-2913, 1995.
Ghandour, M. S.; Langley, O. K.; Zhu, X. L.; Waheed, A.; Sly, W. S.: Carbonic anhydrase IV on brain capillary endothelial cells: a marker associated with the blood-brain barrier. Proc. Nat. Acad. Sci. 89: 6823-6827, 1992.
Hageman, G. S.; Zhu, X. L.; Waheed, A.; Sly, W. S.: Localization of carbonic anhydrase IV in a specific capillary bed of the human eye. Proc. Nat. Acad. Sci. 88: 2716-2720, 1991.
Okuyama, T.; Batanian, J. R.; Sly, W. S.: Genomic organization and localization of gene for human carbonic anhydrase IV to chromosome 17q. Genomics 16: 678-684, 1993.
Okuyama, T.; Sato, S.; Zhu, X. L.; Waheed, A.; Sly, W. S.: Human carbonic anhydrase IV: cDNA cloning, sequence comparison, and expression in COS cell membranes. Proc. Nat. Acad. Sci. 89: 1315-1319, 1992.
Sender, S.; Gros, G.; Wahleed, A.; Hageman, G. S.; Sly, W. S.: Immunohistochemical localization of carbonic anhydrase IV in capillaries of rat and human skeletal muscle. J. Histochem. Cytochem. 42: 1229-1236, 1994.
Wagner, F. F.; Flegel, W. A.: RHD gene deletion occurred in the Rhesus box. Blood 95: 3662-3668, 2000.
Roychoudhury, A. K.; Nei, M.: Human Polymorphic Genes: World Distribution. New York: Oxford Univ. Press (pub.) 1988.
Rodriguez de Cordoba, S.; Lublin, D. M.; Rubinstein, P.; Atkinson, J. P.: Human genes for three complement components that regulate the activation of C3 are tightly linked. J. Exp. Med. 161: 1189-1195, 1985.
Lee, R. T.; Peterson, C. L.; Calman, A. F.; Herskowitz, I.; O'Donnell, J. J.: Cloning of a human galacto kinase gene (GK2) on chromosome 15 by complementation in yeast. Proc. Nat. Acad. Sci. 89: 10887-10891, 1992.
Pastuszak, I.; O'Donnell, J.; Elbein, A. D.: Identification of the GalNAc kinase amino acid sequence. J. Biol. Chem. 271: 23653-23656, 1996.
Synold, T. W.; Dussault, I.; Forman, B. M.: The orphan nuclear receptor SXR coordinately regulates drug metabolism and efflux. Nature Med. 7: 584-590, 2001.
Woods, K. A.; Camacho-Hubner, C.; Bergman, R. N.; Barter, D.; Clark, A. J. L.; Savage, M. O.: Effects of insulin-like growth factor I (IGF-1) therapy on body composition and insulin resistance in IGF-I gene deletion. J. Clin. Endocr. Metab. 85: 1407-1411, 2000.
Woods, K. A.; Camacho-Hubner, C.; Savage, M. O.; Clark, A. J. L.: Intrauterine growth retardation and postnatal growth failure associated with deletion of the insulin-like growth factor I gene. New Eng. J. Med. 335: 1363-1367, 1996.
Yang-Feng, T. L.; Brissenden, J. E.; Ullrich, A.; Francke, U.: Sub-regional localization of human genes for insulin-like growth factors I (IGF1) and II (IGF2) by in situ hybridization. (Abstract) Cytogenet. Cell Genet. 40: 782 only, 1985.
Yanovski, J. A.; Sovik, K. N.; Nguyen, T. T.; Sebring, N. G.: Insulin-like growth factors and bone mineral density in African American and white girls. J. Pediat. 137: 826-832, 2000.
Zhu, J.; Kahn, C. R.: Analysis of a peptide hormone-receptor interaction in the yeast two-hybrid system. Proc. Nat. Acad. Sci. 94:13063-13068, 1997
Kobayashi, M.; Takamatsu, K.; Saitoh, S.; Miura, M.; Noguchi, T.: Molecular cloning of hippocalcin, a novel calcium-binding protein of the recover in family exclusively expressed in hippocampus. Biochem. Biophys. Res. Commun. 189: 511-517, 1992.
Takamatsu, K.; Kobayashi, M.; Saitoh, S.; Fujishiro, M.; Noguchi, T.: Molecular cloning of human hippocalcin cDNA and chromosomal mapping of its gene. Biochem. Biophys. Res. Commun. 200: 606-611, 1994.
Borrego, S.; Ruiz, A.; Saez, M. E.; Gimm, O.; Gao, X.; Lopez-Alonso, M.; Hernandez, A.; Wright, F. A.; Antinolo, G.; Eng, C.: RET genotypes comprising specific haplotypes of polymorphic variants predispose to isolated Hirschsprung disease. J. Med. Genet. 37: 572-578, 2000.
Borrego, S.; Saez, M. E.; Ruiz, A.; Gimm, O.; Lopez-Alonso, M.; Antinolo, G.; Eng, C.: Specific polymorphisms in the RET proto-oncogene are over-represented in patients with Hirschsprung disease and may represent loci modifying phenotypic expression. J. Med. Genet. 36:771-774, 1999.
Iwashita, T.; Murakami, H.; Asai, N.; Takahashi, M.: Mechanism of Ret dysfunction by Hirschsprung mutations affecting its extracellular domain. Hum. Molec. Genet. 5: 1577-1580, 1996
Huguet, E. L.; McMahon, J. A.; McMahon, A. P.; Bicknell, R.; Harris, A. L.: Differential expression of human Wnt genes 2, 3, 4, and 7Bin human breast cell lines and normal and disease states of human breast tissue. Cancer Res. 54: 2615-2621, 1994.
Nusse, R.; Brown, A.; Papkoff, J.; Scambler, P.; Shackleford, G.; McMahon, A.; Moon, R.; Varmus, H.: A new nomenclature for int-1 and related genes: the Wnt gene family. Cell 64: 231-232, 1991.
Yamauchi, M.; Yamauchi, N.; Phear, G.; Spurr, N. K.; Martinsson, T.; Weith, A.; Meuth, M.: Genomic organization and chromosomal localization of the human CTP synthetase gene (CTPS). Genomics 11: 1088-1096, 1991.
Courseaux, A.; Grosgeorge, J.; Gaudray, P.; Pannett, A. A. J.; Forbes, S. A.; Williamson, C.; Bassett, D.; Thakker, R. V.; Teh, B. T.; Farnebo, F.; Shepherd, J.; Skogseid, B.; Larsson, C.; Giraud, S.; Zhang, C. X.; Salandre, J.; Calender, A.: Definition of the minimal MEN1 candidate area based on a 5-Mb integrated map of proximal 11q13. Genomics 37:354-365, 1996.
Dickson, K. M.; Bergeron, J. J. M.; Shames, I.; Colby, J.; Nguyen, D. T.; Chevet, E.; Thomas, D. Y.; Snipes, G. J.: Association of calnexin with mutant peripheral myelin protein-22 ex vivo: a basis for ‘gain-of-function’ ER diseases. Proc. Nat. Acad. Sci. 99: 9852-9857, 2002.
Gray, P. W.; Byers, M. G.; Eddy, R. L.; Shows, T. B.: The assignment of the calnexin gene to the q35 region of chromosome 5. (Abstract) Human Genome Mapping Workshop 93 9 only, 1993.
Schrag, J. D.; Bergeron, J. J. M.; Li, Y.; Borisova, S.; Hahn, M.; Thomas, D. Y.; Cygler, M.: The structure of calnexin, an ER chaperone involved in quality control of protein folding. Molec. Cell 8: 633-644, 2001.
Tjoelker, L. W.; Seyfried, C. E.; Eddy, R. L., Jr.; Byers, M. G.; Shows, T. B.; Calderon, J.; Schreiber, R. B.; Gray, P. W.: Human, mouse, and rat calnexin cDNA cloning: identification of potential calcium binding motifs and gene localization to human chromosome 5. Biochemistry 33:3229-3236, 1994.
Cyr, C.; Huebner, K.; Druck, T.; Kris, R.: Cloning and chromosomal localization of a human endothelin ETA receptor. Biochem. Biophys. Res. Commun. 181: 184-190, 1991.
Hosoda, K.; Nakao, K.; Tamura, N.; Arai, H.; Ogawa, Y.; Suga, S.; Nakanishi, S.; Imura, H.: Organization, structure, chromosomal assignment, and expression of the gene encoding the human endothelin-A receptor. J. Biol. Chem. 267: 18797-18804, 1992.
Tzourio, C.; E1 Amrani, M.; Poirier, O.; Nicaud, V.; Bousser, M.-G.; Alperovitch, A.: Association between migraine and endothelin type A receptor (ETA-231 A/G) gene polymorphism. Neurology 56: 1273-1277, 2001.
Carrasquillo, M. M.; McCallion, A. S.; Puffenberger, E. G.; Kashuk, C. S.; Nouri, N.; Chakravarti, A.: Genome-wide association study and mouse model identify interaction between RET and EDNRB pathways in Hirschsprung disease. Nature Genet. 32: 237-244, 2002.
Denker, S. P.; Huang, D. C.; Orlowski, J.; Furthmayr, H.; Barber, D. L.: Direct binding of the Na—H exchanger NHE1 to ERM proteins regulates the cortical cytoskeleton and cell shape independently of H(+) translocation. Molec. Cell 6: 1425-1436, 2000.
Dudley, C. R. K.; Giuffra, L. A.; Tippett, P.; Kidd, K. K.; Reeders, S. T.: The Na+/H+ antiporter: a ‘melt’ polymorphism allows regional mapping to the short arm of chromosome 1. Hum. Genet. 86: 79-83, 1990.
Franchi, A.; Perucca-Lostanlen, D.; Pouyssegur, J.: Functional expression of a human Na+/H+ antiporter gene transfected into antiporter-deficient mouse L cells. Proc. Nat. Acad. Sci. 83: 9388-9392, 1986
Lifton, R. P.; Sardet, C.; Pouyssegur, J.; Lalouel, J.-M.: Cloning of the human genomic amiloride-sensitive Na+/H+ antiporter gene, identification of genetic polymorphisms, and localization on the genetic map of chromosome 1p. Genomics 7: 131-135, 1990.
Mattei, M.-G.; Galloni, M.; Sardet, C.; Franchi, A.; Counillon, L.; Passage, E.; Pouyssegur, J.: Localization of the antiporter gene (APNH) and chromosomal homology between human 1p, mouse 4 and Chinese hamster 2q. (Abstract) Cytogenet. Cell Genet. 51: 1041, 1989.
Mattei, M.-G.; Sardet, C.; Franchi, A.; Pouyssegur, J.: Chromosomal mapping of the amiloride-sensitive Na+/H+ antiporter gene. (Abstract) Cytogenet. Cell Genet. 46: 658-659, 1987.
Mattei, M.-G.; Sardet, C.; Franchi, A.; Pouyssegur, J.: The human amiloride-sensitive Na+/H+ antiporter: localization to chromosome 1 by in situ hybridization. Cytogenet. Cell Genet. 48: 6-8, 1988.
Mendoza, S. A.: The Na+/H+ antiport is a mediator of cell proliferation. Acta Paediat. Scand. 76: 545-547, 1987.
Morahan, G.; Rakar, S.: Localization of the mouse Na+/H+ exchanger gene on distal chromosome 4. Genomics 15: 231-232, 1993
Sardet, C.; Franchi, A.; Pouyssegur, J.: Molecular cloning, primary structure, and expression of the human growth factor-activatable Na(+)/H(+)antiporter. Cell 56: 271-280, 1989.
Whitney, P. L.; Briggle, T. V.: Membrane-associated carbonic anhydrase purified from bovine lung. J. Biol. Chem. 257: 12056-12059, 1982.
Zhu, X. L.; Sly, W. S.: Carbonic anhydrase IV from human lung: purification, characterization, and comparison with membrane carbonic anhydrase from human kidney. J. Biol. Chem. 265: 8795-8801, 1990.
Iwata, N.; Tsubuki, S.; Takaki, Y.; Shirotani, K.; Lu, B.; Gerard, N. P.; Gerard, C.; Hama, E.; Lee, H.-J.; Saido, T. C.: Metabolic regulation of brain A-beta by neprilysin. Science 292: 1550-1552, 2001.
McGeer, P. L.; McGeer, E. G.: Polymorphisms in inflammatory genes and the risk of Alzheimer disease. Arch. Neurol. 58: 1790-1792, 2001.
Modi, W. S.; Masuda, A.; Yamada, M.; Oppenheim, J. J.; Matsushima, K.; O'Brien, S. J.: Chromosomal localization of the human interleukin 1 alpha (IL-1-alpha) gene. Genomics 2: 310-314, 1988.
Mosley, B.; Urdal, D. L.; Prickett, K. S.; Larsen, A.; Cosman, D.; Conlon, P. J.; Gillis, S.; Dower, S. K.: The interleukin-1 receptor binds the human interleukin-1-alpha precursor but not the interleukin-1-beta precursor. J. Biol. Chem. 262: 2941-2944, 1987.
Bailly, S.; di Giovine, F. S.; Blakemore, A. I. F.; Duff, G. W.: Genetic polymorphism of human interleukin-1-alpha. Europ. J. Immun. 23:1240-1245, 1993.
Boultwood, J.; Breckon, G.; Birch, D.; Cox, R.: Chromosomal localization of murine interleukin-1 alpha and beta genes. Genomics 5: 481-485, 1989.
Cox, A.; Camp, N. J.; Cannings, C.; di Giovine, F. S.; Dale, M.; Worthington, J.; John, S.; Ollier, W. E. R.; Silman, A. J.; Duff, G. W.: Combined sib-TDT and TDT provide evidence for linkage of the interleukin-1 gene cluster to erosive rheumatoid arthritis. Hum. Molec. Genet. 8: 1707-1713, 1999.
Cox, A.; Camp, N. J.; Nicklin, M. J. H.; di Giovine, F. S.; Duff, G. W.: An analysis of linkage disequilibrium in the interleukin-1 gene cluster, using a novel grouping method for multiallelic markers. Am. J. Hum. Genet. 62: 1180-1188, 1998.
Diehl, S. R.; Wang, Y.; Brooks, C. N.; Burmeister, J. A.; Califano, J. V.; Wang, S.; Schenkein, H. A.: Linkage disequilibrium of interleukin-1 genetic polymorphisms with early-onset periodontitis. J. Periodont. 70:418-430, 1999.
Du, Y.; Dodel, R. C.; Eastwood, B. J.; Bales, K. R.; Gao, F.; Lohmuller, F.; Muller, U.; Kurz, A.; Zimmer, R.; Evans, R. M.; Hake, A.; Gasser, T.; Oertel, W. H.; Griffin, W. S. T.; Paul, S. M.; Farlow, M. R.: Association of an interleukin 1-alpha polymorphism with Alzheimer's disease. Neurology 55: 480-484, 2000.
Furutani, Y.; Notake, M.; Fukui, T.; Ohue, M.; Nomura, H.; Yamada, M.; Nakamura, S.: Complete nucleotide sequence of the gene for human interleukin 1 alpha. Nucleic Acids Res. 14: 3167-3179, 1986.
Gray, P. W.; Glaister, D.; Chen, E.; Goeddel, D. V.; Pennica, D.: Two interleukin 1 genes in the mouse: cloning and expression of the cDNA for murine interleukin 1-beta. J. Immun. 137: 3644-3648, 1986.
Grimaldi, L. M. E.; Casadei, V. M.; Ferri, C.; Veglia, F.; Licastro, F.; Annoni, G.; Biunno, I.; De Bellis, G.; Sorbi, S.; Mariani, C.; Canal, N.; Griffin, W. S. T.: Association of early-onset Alzheimer's disease with an interleukin-1-alpha gene polymorphism. Ann. Neurol. 47:361-365, 2000.
Hogquist, K. A.; Nett, M. A.; Unanue, E. R.; Chaplin, D. D.: Interleukin 1 is processed and released during apoptosis. Proc. Nat. Acad. Sci. 88: 8485-8489, 1991.
Hurwitz, A.; Loukides, J.; Ricciarelli, E.; Botero, L.; Katz, E.; McAllister, J. M.; Garcia, J. E.; Rohan, R.; Adashi, E. Y.; Hernandez, E. R.: Human intraovarian interleukin-1 (IL-1) system: highly compartmentalized and hormonally dependent regulation of the genes encoding IL-1, its receptor, and its receptor antagonist. J. Clin. Invest. 89: 1746-1754, 1992.
Ki, C.-S.; Na, D. L.; Kim, D. K.; Kim, H. J.; Kim, J.-W.: Lack of association of the interleukin-1-alpha gene polymorphism with Alzheimer's disease in a Korean population. (Letter) Ann. Neurol. 49: 817-818, 2001.
Kolsch, H.; Ptok, U.; Bagli, M.; Papassotiropoulos, A.; Schmitz, S.; Barkow, K.; Kockler, M.; Rao, M. L.; Maier, W.; Heun, R.: Gene polymorphisms of interleukin-1-alpha influence the course of Alzheimer's disease. (Letter) Ann. Neurol. 49: 818-819, 2001.
Kornman, K. S.; Crane, A.; Wang, H.-Y.; di Giovine, F. S.; Newman, M. G.; Pirk, F. W.; Wilson, T. G., Jr.; Higginbottom, F. L.; Duff, G. W.: The interleukin-1 genotype as a severity factor in adult periodontal disease. J. Clin. Periodont. 24: 72-77, 1997.
Lafage, M.; Maroc, N.; Dubreuil, P.; de Waal Malefijt, R.; Pebusque, M.-J.; Carcassonne, Y.; Mannoni, P.: The human interleukin-1-alpha gene is located on the long arm of chromosome 2 at band q13. Blood 73:104-107, 1989.
Lord, P. C. W.; Wilmoth, L. M. G.; Mizel, S. B.; McCall, C. E.: Expression of interleukin-1 alpha and beta genes by human blood polymorphonuclear leukocytes. J. Clin. Invest. 87: 1312-1321, 1991.
Murphy, G. M., Jr.; Claassen, J. D.; DeVoss, J. J.; Pascoe, N.; Taylor, J.; Tinklenberg, J. R.; Yesavage, J. A.: Rate of cognitive decline in AD is accelerated by the interleukin-1-alpha-889 *1 allele. Neurology 56:1595-1597, 2001.
Motyckova, G.; Weilbaecher, K. N.; Horstmann, M.; Rieman, D. J.; Fisher, D. Z.; Fisher, D. E.: Linking osteopetrosis and pycnodysostosis: regulation of cathepsin K expression by the microphthalmia transcription factor family. Proc. Nat. Acad. Sci. 98: 5798-5803, 2001.
Guillon, H.; de Massy, B.: An initiation site for meiotic crossing-over and gene conversion in the mouse. Nature Genet. 32: 296-299, 2002.
Parolini, S.; Bottino, C.; Falco, M.; Augugliaro, R.; Giliani, S.; Franceschini, R.; Ochs, H. D.; Wolf, H.; Bonnefoy, J.-Y.; Biassoni, R.; Moretta, L.; Notarangelo, L. D.; Moretta, A.: X-linked lymphoproliferative disease: 2B4 molecules displaying inhibitory rather than activating function are responsible for the inability of natural killer cells to kill Epstein-Barr virus-infected cells. J. Exp. Med. 192: 337-346, 2000.
McPherron, A. C.; Lee, S.-J.: Suppression of body fat accumulation in myostatin-deficient mice. J. Clin. Invest. 109: 595-601, 2002.
Attisano, L.; Carcamo, J.; Ventura, F.; Weis, F. M. B.; Massague, J.; Wrana, J. L.: Identification of human activin and TGF-beta type I receptors that form heteromeric kinase complexes with type II receptors. Cell 75:671-680, 1993.
Mathews, L. S.; Vale, W. W.: Expression cloning of an activin receptor, a predicted transmembrane serine kinase. Cell 65: 973-982, 1991.
Matsuzaki, K.; Xu, J.; Wang, F.; McKeehan, W. L.; Krummen, L.; Kan, M.: A widely expressed transmembrane serine/threonine kinase that does not bind activin, inhibin, transforming growth factor beta, or bone morphogenic factor. J. Biol. Chem. 268: 12719-12723, 1993.
Roijer, E.; Miyazono, K.; Astrom, A.-K.; Geurts van Kessel, A.; ten Dijke, P.; Stenman, G.: Chromosomal localization of three human genes encoding members of the TGF-beta super family of type I serine/threonine kinase receptors. Mammalian Genome 9: 266-268, 1998.
ten Dijke, P.; Ichijo, H.; Franzen, P.; Schulz, P.; Saras, J.; Toyoshima, H.; Heldin, C.-H.; Miyazono, K.: Activin receptor-like kinases: a novel subclass of cell-surface receptors with predicted serine/threonine kinase activity. Oncogene 8: 2879-2887, 1993.
Ferguson-Smith, A. C.; Cattanach, B. M.; Barton, S. C.; Beechey, C. V.; Surani, M. A.: Embryological and molecular investigations of parental imprinting on mouse chromosome 7. Nature 351: 667-670, 1991.
Chang, T.-M.; Neville, D. M., Jr.: Demonstration of diphtheria toxin receptors on surface membranes from both toxin-sensitive and toxin-resistant species. J. Biol. Chem. 253: 6866-6871, 1978.
Creagan, R. P.; Chen, S.-H.; Ruddle, F. H.: Genetic analysis of the cell surface: association of human chromosome 5 with sensitivity to diphtheria toxin in mouse-human somatic cell hybrids. Proc. Nat. Acad. Sci. 72: 2237-2241, 1975.
Fen, Z.; Dhadly, M. S.; Yoshizumi, M.; Hilkert, R. J.; Quertermous, T.; Eddy, R. L.; Shows, T. B.; Lee, M.-E.: Structural organization and chromosomal assignment of the gene encoding the human heparin-binding epidermal growth factor-like growth factor/diphtheria toxin receptor. Biochemistry 32:7932-7938, 1993.
George, D. L.; Francke, U.: Regional mapping of human genes for hexosaminidase B and diphtheria toxin sensitivity on chromosome 5 using mouse X human hybrid cells. Somat. Cell Genet. 3: 629-638, 1977.
Gupta, R. S.; Siminovitch, L.: Isolation and characterization of mutants of human diploid fibroblasts resistant to diphtheria toxin. Proc. Nat. Acad. Sci. 75: 3337-3340, 1978.
Hayes, H.; Kaneda, Y.; Uchida, T.; Okada, Y.: Regional assignment of the gene for diphtheria toxin sensitivity using subchromosomal fragments in micro cell hybrids. Chromosoma 96: 26-32, 1987.
Higashiyama, S.; Abraham, J. A.; Miller, J.; Fiddes, J. C.; Klagsbrun, M.: A heparin-binding growth factor secreted by macrophage-like cells that is related to EGF. Science 251: 936-939, 1991.
Demetrick, D. J.; Matsumoto, S.; Hannon, G. J.; Okamoto, K.; Xiong, Y.; Zhang, H.; Beach, D. H.: Chromosomal mapping of the genes for the human cell cycle proteins cyclin C (CCNC), cyclin E (CCNE), p21(CDKN1) and KAP (CDKN3). Cytogenet. Cell Genet. 69: 190-192, 1995.
Buonavista, N.; Balzano, C.; Pontarotti, P.; Le Paslier, D.; Golstein, P.: Molecular linkage of the human CTLA4 and CD28 Ig-superfamily genes in yeast artificial chromosomes. Genomics 13: 856-861, 1992.
Howard, T. A.; Rochelle, J. M.; Seldin, M. F.: Cd28 and Ctla-4, two related members of the Ig super gene family, are tightly linked on proximal mouse chromosome 1. Immunogenetics 33: 74-76, 1991.
Gallagher, P. G.; Forget, B. G.: Structure, organization, and expression of the band 7.2b gene, a candidate gene for hereditary hydrocytosis. J. Biol. Chem. 270: 26358-26363, 1995.
Gallagher, P. G.; Romana, M.; Lieman, J. H.; Ward, D. C.: cDNA structure, tissue-specific expression, and chromosomal localization of the murine band 7.2b gene. Blood 86: 359-365, 1995.
Gallagher, P. G.; Upender, M.; Ward, D. C.; Forget, B. G.: The gene for human erythrocyte membrane protein band 7.2 (EPB72) maps to 9q33-q34 centromeric to the Philadelphia chromosome translocation breakpoint region. Genomics 18: 167-169, 1993.
Hiebl-Dirschmied, C. M.; Entler, B.; Glotzmann, C.; Maurer-Fogy, I.; Stratowa, C.; Prohaska, R.: Cloning and nucleotide sequence of cDNA encoding human erythrocyte band 7 integral membrane protein. Biochim. Biophys. Acta 1090: 123-124, 1991.
Pilz, A.; Prohaska, R.; Peters, J.; Abbott, C.: Genetic linkage analysis of the Ak1, Col5a1, Epb7.2, Fpgs, Grp78, Pbx3, and Notch1 genes in the region of mouse chromosome 2 homologous to human chromosome 9q. Genomics 21: 104-109, 1994.
Unfried, I.; Entler, B.; Prohaska, R.: The organization of the gene (EPB72) encoding the human erythrocyte band 7 integral membrane protein (protein 7.2b). Genomics 30: 521-528, 1995.
Westberg, J. A.; Entler, B.; Prohaska, R.; Schroder, J. P.: The gene coding for erythrocyte protein band 7.2b (EPB72) is located inband q34.1 of human chromosome 9. Cytogenet. Cell Genet. 63: 241-243, 1993.
Zhu, Y.; Paszty, C.; Turetsky, T.; Tsai, S.; Kuypers, F. A.; Lee, G.; Cooper, P.; Gallagher, P. G.; Stevens, M. E.; Rubin, E.; Mohandas, N.; Mentzer, W. C.: Stomatocytosis is absent in ‘stomatin’-deficient murine red blood cells. Blood 93: 2404-2410, 1999.
Lacy, S. E.; Bonnemann, C. G.; Buzney, E. A.; Kunkel, L. M.: Identification of FLRT1, FLRT2, and FLRT3: a novel family of transmembrane leucine-rich repeat proteins. Genomics 62: 417-426, 1999.
Horikoshi, N.; Cong, J.; Kley, N.; Shenk, T.: Isolation of differentially expressed cDNAs from p53-dependent apoptotic cells: activation of the human homologue of the Drosophila peroxidasin gene. Biochem. Biophys. Res. Commun. 261: 864-869, 1999.
Weiler, S. R.; Taylor, S. M.; Deans, R. J.; Kan-Mitchell, J.; Mitchell, M. S.; Trent, J. M.: Assignment of a human melanoma associated gene MG50 (D2S448) to chromosome 2p25.3 by fluorescence in situ hybridization. Genomics 22:243-244, 1994.
Betz, R.; Gray, S. G.; Ekstrom, C.; Larsson, C.; Ekstrom, T. J.: Human histone deacetylase 2, HDAC2 (human RPD3), is localized to 6q21 by radiation hybrid mapping. Genomics 52: 245-246, 1998.
Inouye, C. J.; Seto, E.: Relief of YY1-induced transcriptional repression by protein-protein interaction with the nuclear phosphoprotein B23. J. Biol. Chem. 269: 6506-6510, 1994.
Randhawa, G. S.; Bell, D. W.; Testa, J. R.; Feinberg, A. P.: Identification and mapping of human histone acetylation modifier gene homologues. Genomics 51:262-269, 1998.
Yang, W.-M.; Inouye, C.; Zeng, Y.; Bearss, D.; Seto, E.: Transcriptional repression by YY1 is mediated by interaction with a mammalian homolog of the yeast global regulator RPD3. Proc. Nat. Acad. Sci. 93: 12845-12850, 1996.
Yarden, R. I.; Brody, L. C.: BRCA1 interacts with components of the histone deacetylase complex. Proc. Nat. Acad. Sci. 96: 4983-4988, 1999.
Dermaut, B.; Theuns, J.; Sleegers, K.; Hasegawa, H.; Van den Broeck, M.; Vennekens, K.; Corsmit, E.; St. George-Hyslop, P.; Cruts, M.; van Duijn, C. M.; Van Broeckhoven, C.: The gene encoding nicastrin, a major gamma-secretase component, modifies risk for familial early-onset Alzheimer disease in a Dutch population-based sample. Am. J. Hum. Genet. 70: 1568-1574, 2002.
Feldman, R. G.; Chandler, K. A.; Levy, L. L.; Glaser, G. H.: Familial Alzheimer's disease. Neurology 13: 811-824, 1963.
Foncin, J.-F.; Salmon, D.; Supino-Viterbo, V.; Feldman, R. G.; Macchi, G.; Mariotti, P.; Scoppetta, C.; Caruso, G.; Bruni, A. C.: Demence presenile d'Alzheimer transmise dans une famille etendue. Rev. Neurol. (Paris) 141: 194-202, 1985.
Hiltunen, M.; Mannermaa, A.; Thompson, D.; Easton, D.; Pirskanen, M.; Helisalmi, S.; Koivisto, A. M.; Lehtovirta, M.; Ryynanen, M.; Soininen, H.: Genome-wide linkage disequilibrium mapping of late-onset Alzheimer's disease in Finland. Neurology 57: 1663-1668, 2001.
Kehoe, P.; Wavrant-De Vrieze, F.; Crook, R.; Wu, W. S.; Holmans, P.; Fenton, I.; Spurlock, G.; Norton, N.; Williams, H.; Williams, N.; Lovestone, S.; Perez-Tur, J.; Hutton, J.; and 10 others: A full genome scan for late onset Alzheimer disease. Hum. Molec. Genet. 8:237-245, 1999.
Kopan, R.; Goate, A.: Aph-2/nicastrin: an essential component of gamma-secretase and regulator of Notch signaling and presenilin localization. Neuron 33: 321-324, 2002.
Yu, G.; Nishimura, M.; Arawaka, S.; Levitan, D.; Zhang, L.; Tandon, A.; Song, Y.-Q.; Rogaeva, E.; Chen, F.; Kawarai, T.; Supala, A.; Levesque, L.; and 18 others: Nicastrin modulates presenilin-mediated notch/glp-1 signal transduction and beta-APP processing. Nature 407: 48-54, 2000.
Zubenko, G. S.; Hughes, H. B.; Stiffler, J. S.; Hurtt, M. R.; Kaplan, B. B.: A genome survey for novel Alzheimer disease risk loci: results at 10-cM resolution. Genomics 50: 121-128, 1998.
Arakawa, H.; Hauschild, J.; Buerstedde, J.-M.: Requirement of the activation-induced deaminase (AID) gene for immunoglobulin gene conversion. Science 295: 1301-1306, 2002.
Fagarasan, S.; Kinoshita, K.; Muramatsu, M.; Ikuta, K.; Honjo, T.: In situ class switching and differentiation to IgA producing cells in the gut lamina propria. Nature 413: 639-643, 2001.
Muramatsu, M.; Kinoshita, K.; Fagarasan, S.; Yamada, S.; Shinkai, Y.; Honjo, T.: Class switch recombination and hypermutation require activation-induced cytidine deaminase (AID), a potential RNA editing enzyme. Cell 102: 553-563, 2000.
Muramatsu, M.; Sankaranand, V. S.; Anant, S.; Sugai, M.; Kinoshita, K.; Davidson, N. O.; Honjo, T.: Specific expression of activation-induced cytidine deaminase (AID), a novel member of the RNA-editing deaminase family in germinal center B cells. J. Biol. Chem. 274: 18470-18476, 1999.
Bak, M.; Hansen, C.; Henriksen, K. F.; Tommerup, N.: The human hedgehog-interacting protein gene: structure and chromosome mapping to 4q31.21-q31.3. Cytogenet. Cell Genet. 92: 300-303, 2001.
Chuang, P.-T.; McMahon, A. P.: Vertebrate hedgehog signalling modulated by induction of a hedgehog-binding protein. Nature 397:617-621, 1999
Nakagawa, T.; Setou, M.; Seog, D.-H.; Ogasawara, K.; Dohmae, N.; Takio, K.; Hirokawa, N.: A novel motor, KIF13A, transports mannose-6-phosphate receptor to plasma membrane through direct interaction with AP-1 complex. Cell 103:569-581, 2000.
Scott, A. F.: Personal Communication. Baltimore, Md. Nov. 29, 2000.
Blumberg, H.; Conklin, D.; Xu, W.; Grossmann, A.; Brender, T.; Carollo, S.; Eagan, M.; Foster, D.; Haldeman, B. A.; Hammond, A.; Haugen, H.; Jelinek, L.; and 14 others: Interleukin 20: discovery, receptor identification, and role in epidermal function. Cell 104:9-19, 2001.
Armstrong, E.; Partanen, J.; Cannizzaro, L.; Huebner, K.; Alitalo, K.: Localization of the fibroblast growth factor receptor-4 geneto chromosome region 5q33-qter. Genes Chromosomes Cancer 4: 94-98, 1992.
Bange, J.; Prechtl, D.; Cheburkin, Y.; Specht, K.; Harbeck, N.; Schmitt, M.; Knyazeva, T.; Muller, S.; Gartner, S.; Sures, I.; Wang, H.; Imyanitov, E.; Haring, H.-U.; Knayzev, P.; Iacobelli, S.; Hofler, H.; Ullrich, A.: Cancer progression and tumor cell motility are associated with the FGFR4 Arg388 allele. Cancer Res. 62: 840-847, 2002.
Holtrich, U.; Brauninger, A.; Strebhardt, K.; Rubsamen-Waigmann, H.: Two additional protein-tyrosine kinases expressed in human lung: fourth member of the fibroblast growth factor receptor family and an intracellular protein-tyrosine kinase. Proc. Nat. Acad. Sci. 88:10411-10415, 1991.
Kostrzewa, M.; Muller, U.: Genomic structure and complete sequence of the human FGFR4 gene. Mammalian Genome 9: 131-135, 1998.
Partanen, J.; Makela, T. P.; Eerola, E.; Korhonen, J.; Hirvonen, H.; Claesson-Welsh, L.; Alitalo, K.: FGFR-4, a novel acidic fibroblast growth factor receptor with a distinct expression pattern. EMBO J. 10:1347-1354, 1991.
Scott, A. F.: Personal Communication. Baltimore, Md. Oct. 12, 1999.
Vainikka, S.; Partanen, J.; Bellosta, P.; Coulier, F.; Basilico, C.; Jaye, M.; Alitalo, K.: Fibroblast growth factor receptor-4 shows novel features in genomic structure, ligand binding and signal transduction. EMBO J. 11: 4273-4280, 1992.
Warrington, J. A.; Bailey, S. K.; Armstrong, E.; Aprelikova, O.; Alitalo, K.; Dolganov, G. M.; Wilcox, A. S.; Sikela, J. M.; Wolfe, S. F.; Lovett, M.; Wasmuth, J. J.: A radiation hybrid map of 18 growth factor, growth factor receptor, hormone receptor, or neurotransmitter receptor genes on the distal region of the long arm of chromosomes. Genomics 13: 803-808, 1992.
Ogihara, T.; Isobe, T.; Ichimura, T.; Taoka, M.; Funaki, M.; Sakoda, H.; Onishi, Y.; Inukai, K.; Anai, M.; Fukushima, Y.; Kikuchi, M.; Yazaki, Y.; Oka, Y.; Asano, T.: 14-3-3 protein binds to insulin receptor substrate-1, one of the binding sites of which is in the phosphotyrosine binding domain. J. Biol. Chem. 272: 25267-25274, 1997.
Kumar, S.; McDonnell, P. C.; Lehr, R.; Tierney, L.; Tzimas, M. N.; Griswold, D. E.; Capper, E. A.; Tal-Singer, R.; Wells, G. I.; Doyle, M. L.; Young, P. R.: Identification and initial characterization of four novel members of the interleukin-1 family. J. Biol. Chem. 275:10308-10314, 2000.
Torrents, D.; Estevez, R.; Pineda, M.; Fernandez, E.; Lloberas, J.; Shi, Y.-B.; Zorzano, A.; Palacin, M.: Identification and characterization of a membrane protein (y(+)L amino acid transporter-1) that associates with 4F2hc to encode the amino acid transport activity y(+)L: a candidate gene for lysinuric protein intolerance. J. Biol. Chem. 273: 32437-32445, 1998.
Cohen-Salmon, M.; Frenz, D.; Liu, W.; Verpy, E.; Voegeling, S.; Petit, C.: Fdp, a new fibrocyte-derived protein related to MIA/CD-RAP, has an in vitro effect on the early differentiation of the inner earmesenchyme. J. Biol. Chem. 275: 40036-40041, 2000.
Rendtorff, N. D.; Frodin, M.; Attie-Bitach, T.; Vekemans, M.; Tommerup, N.: Identification and characterization of an inner ear-expressed human melanoma inhibitory activity (MIA)-like gene (MIAL) with a frequent polymorphism that abolishes translation. Genomics 71: 40-52, 2001.
Robertson, N. G.; Heller, S.; Lin, J. S.; Resendes, B. L.; Weremowicz, S.; Denis, C. S.; Bell, A. M.; Hudspeth, A. J.; Morton, C. C.: A novel conserved cochlear gene, OTOR: identification, expression analysis, and chromosomal mapping. Genomics 66: 242-248, 2000.
Busfield, S. J.; Comrack, C. A.; Yu, G.; Chickering, T. W.; Smutko, J. S.; Zhou, H.; Leiby, K. R.; Holmgren, L. M.; Gearing, D. P.; Pan, Y.: Identification and gene organization of three novel members of the IL-1 family on human chromosome 2. Genomics 66: 213-216, 2000.
Matsuyoshi, N.; Tanaka, T.; Toda, K.; Imamura, S.: Identification of novel cadherins expressed in human melanoma cells. J. Invest. Derm. 108: 908-913, 1997.
Li, Y.; Chin, L.-S.; Weigel, C.; Li, L.: Spring, a novel RING finger protein that regulates synaptic vesicle exocytosis. J. Biol. Chem. 276: 40824-40833, 2001.
Reymond, A.; Meroni, G.; Fantozzi, A.; Merla, G.; Cairo, S.; Luzi, L.; Riganelli, D.; Zanaria, E.; Messali, S.; Cainarca, S.; Guffanti, A.; Minucci, S.; Pelicci, P. G.; Ballabio, A.: The tripartite motif family identifies cell compartments. EMBO J. 20: 2140-2151, 2001.
Scardigli, R.; Schuurmans, C.; Gradwohl, G.; Guillemot, F.: Cross regulation between neurogenin 2 and pathways specifying neuronal identity in the spinal cord. Neuron 31: 203-217, 2001.
Habas, R.; Kato, Y.; He, X.: Wnt/Frizzled activation of Rho regulates vertebrate gastrulation and requires a novel Formin homology protein Daam1. Cell 107: 843-854, 2001.
Bradley, K. A.; Mogridge, J.; Mourez, M.; Collier, R. J.; Young, J. A. T.: Identification of the cellular receptor for anthrax toxin. Nature 414:160-161, 2001.
Hammarsund, M.; Wilson, W.; Corcoran, M.; Merup, M.; Einhorn, S.; Grander, D.; Sangfelt, O.: Identification and characterization of two novel human mitochondrial elongation factor genes, hEFG2 and hEFG1, phylogenetically conserved through evolution. Hum. Genet. 109: 542-550, 2001
Ishii, H.; Vecchione, A.; Murakumo, Y.; Baldassarre, G.; Numata, S.; Trapasso, F.; Alder, H.; Baffa, R.; Croce, C. M.: FEZ1/LZTS1 gene at 8p22 suppresses cancer cell growth and regulates mitosis. Proc. Nat. Acad. Sci. 98: 10374-10379, 2001.
Bruick, R. K.; McKnight, S. L.: A conserved family of prolyl-4-hydroxylases that modify HIF. Science 294: 1337-1340, 2001.
Epstein, A. C. R.; Gleadle, J. M.; McNeill, L. A.; Hewitson, K. S.; O'Rourke, J.; Mole, D. R.; Mukherji, M.; Metzen, E.; Wilson, M. I.; Dhanda, A.; Tian, Y.-M.; Masson, N.; Hamilton, D. L.; Jaakkola, P.; Barstead, R.; Hodgkin, J.; Maxwell, P. H.; Pugh, C. W.; Schofield, C. J.; Ratcliffe, P. J.: C. elegans EGL-9 and mammalian homologs define a family of dioxygenases that regulate HIF by prolyl hydroxylation. Cell 107:43-54, 2001.
Bleul, C. C.; Farzan, M.; Choe, H.; Parolin, C.; Clark-Lewis, I.; Sodroski, J.; Springer, T. A.: The lymphocyte chemoattractant SDF-1 is a ligand for LESTR/fusin and blocks HIV-1 entry. Nature 382:829-833, 1996.
Caruz, A.; Samsom, M.; Alonso, J. M.; Alcami, J.; Baleux, F.; Virelizier, J. L.; Parmentier, M.; Arenzana-Seisdedos, F.: Genomic organization and promoter characterization of human CXCR4 gene. FEBS Lett. 426:271-278, 1998.
Clapham, P. R.; Blanc, D.; Weiss, R. A.: Specific cell surface requirements for the infection of CD4-positive cells by human immunodeficiency virus types 1 and 2 and by Simian immunodeficiency virus. Virology 181:703-715, 1991.
Dragic, T.; Alizon, M.: Different requirements for membrane fusion mediated by the envelopes of human immunodeficiency virus types 1 and 2. J. Virol. 67: 2355-2359, 1993.
Federsppiel, B.; Melhado, I. G.; Duncan, A. M. V.; Delaney, A.; Schappert, K.; Clark-Lewis, I.; Jirik, F. R.: Molecular cloning of the cDNA and chromosomal localization of the gene for a putative seven-transmembrane segment (7-TMS) receptor isolated from human spleen. Genomics 16:707-712, 1993.
Feng, Y.; Broder, C. C.; Kennedy, P. E.; Berger, E. A.: HIV-1 entry cofactor: functional cDNA cloning of a seven-transmembrane, G protein-coupled receptor. Science 272: 872-876, 1996.
Hendrix, C. W.; Flexner, C.; MacFarland, R. T.; Giandomenico, C.; Fuchs, E. J.; Redpath, E.; Bridger, G.; Henson, G. W.: Pharmacokinetics and safety of AMD-3100, a novel antagonist of the CXCR-4 chemokine receptor, in human volunteers. Antimicrob. Agents Chemother. 44:1667-1673, 2000.
Herzog, H.; Hort, Y. J.; Shine, J.; Selbie, L. A.: Molecular cloning, characterization, and localization of the human homolog to the reported bovine NPY Y3 receptor: lack of NPY binding and activation. DNA Cell Biol. 12: 465-471, 1993.
Jazin, E. E.; Yoo, H.; Blomqvist, A. G.; Yee, F.; Weng, G.; Walker, M. W.; Salon, J.; Larhammar, D.; Wahlestedt, C.: A proposed bovine neuropeptide Y (NPY) receptor cDNA clone, or its human homologue, confers neither NPY binding sites nor NPY responsiveness on transfected cells. Regul. Pept. 47: 247-258, 1993.
Liotta, L. A.: An attractive force in metastasis. Nature 410:24-25, 2001.
Loetscher, M.; Geiser, T.; O'Reilly, T.; Zwahlen, R.; Baggiolini, M.; Moser, B.: Cloning of a human seven-transmembrane domain receptor, LESTR, that is highly expressed in leukocytes. J. Biol. Chem. 269:232-237, 1994.
Lu, M.; Grove, E. A.; Miller, R. J.: Abnormal development of the hippocampal dentate gyrus in mice lacking the CXCR4 chemokine receptor. Proc. Nat. Acad. Sci. 99: 7090-7095, 2002.
Ma, Q.; Jones, D.; Borghesani, P. R.; Segal, R. A.; Nagasawa, T.; Kishimoto, T.; Bronson, R. T.; Springer, T. A.: Impaired B-lymphopoiesis, myelopoiesis, and derailed cerebellar neuron migration in CXCR4— and SDF-1-deficient mice. Proc. Nat. Acad. Sci. 95: 9448-9453, 1998.
Muller, A.; Homey, B.; Soto, H.; Ge, N.; Catron, D.; Buchanan, M. E.; McClanahan, T.; Murphy, E.; Yuan, W.; Wagner, S. N.; Barrera, J. L.; Mohar, A.; Verastegui, E.; Zlotnik, A.: Involvement of chemokine receptors in breast cancer metastasis. Nature 410: 50-56, 2001.
Nagasawa, T.; Hirota, S.; Tachibana, K.; Takakura, N.; Nishikawa, S.; Kitamura, Y.; Yoshida, N.; Kikutani, H.; Kishimoto, T.: Defects of B-cell lymphopoiesis and bone-marrow myelopoiesis in mice lacking the CXC chemokine PBSF/SDF-1. Nature 382: 635-638, 1996.
Oberlin, E.; Amara, A.; Bachelerie, F.; Bessia, C.; Virelizier, J.-L.; Arenzana-Seisdedos, F.; Schwartz, O.; Heard, J.-M.; Clark-Lewis, I.; Legler, D. F.; Loetscher, M.; Baggiollini, M.; Moser, B.: The CXC chemokine SDF-1 is the ligand for LESTR/fusin and prevents infection by T-cell-line-adapted HIV-1. Nature 382: 833-835, 1996.
Peled, A.; Petit, I.; Kollet, O.; Magid, M.; Ponomaryov, T.; Byk, T.; Nagler, A.; Ben-Hur, H.; Many, A.; Shultz, L.; Lider, O.; Alon, R.; Zipori, D.; Lapidot, T.: Dependence of human stem cell engraftment and repopulation of NOD/SCID mice on CXCR4. Science 283: 845-848, 1999.
Rimland, J.; Xin, W.; Sweetnam, P.; Saijoh, K.; Nestler, E. J.; Duman, R. S.: Sequence and expression of a neuropeptide Y receptor cDNA. Molec. Pharm. 40: 869-875, 1991.
Tachibana, K.; Hirota, S.; Iizasa, H.; Yoshida, H.; Kawabata, K.; Kataoka, Y.; Kitamura, Y.; Matsushima, K.; Yoshida, N.; Nishikawa, S.; Kishimoto, T.; Nagasawa, T.: The chemokine receptor CXCR4 isessential for vascularization of the gastrointestinal tract. Nature 393:591-594, 1998.
Wegner, S. A.; Ehrenberg, P. K.; Chang, G.; Dayhoff, D. E.; Sleeker, A. L.; Michael, N. L.: Genomic organization and functional characterization of the chemokine receptor CXCR4, a major entry co-receptor for human immunodeficiency virus type 1. J. Biol. Chem. 273: 4754-4760, 1998.
Weiner, D. B.; Huebner, K.; Williams, W. V.; Greene, M. I.: Human genes other than CD4 facilitate HIV-1 infection of murine cells. Pathobiology 59:361-371, 1991.
Szostecki, C.; Guldner, H. H.; Netter, H. J.; Will, H.: Isolation and characterization of cDNA encoding a human nuclear antigen predominantly recognized by autoantibodies from patients with primary biliary cirrhosis. J. Immun. 145: 4338-4347, 1990.
Liang, Y.; Buckley, T. R.; Tu, L.; Langdon, S. D.; Tedder, T. F.: Structural organization of the human MS4A gene cluster on chromosome 11q12. Immunogenetics 53: 357-368, 2001.
Tedder, T. F.; Disteche, C. M.; Louie, E.; Adler, D. A.; Croce, C. M.; Schlossman, S. F.; Saito, H.: The gene that encodes the human CD20 (B1) differentiation antigen is located on chromosome 11 near the t(11;14)(q13;q32) translocation site. J. Immun. 142: 2555-2559, 1989.
Tedder, T. F.; Klejman, G.; Schlossman, S. F.; Saito, H.: Structure of the gene encoding the human B lymphocyte differentiation antigen CD20 (B1). J. Immun. 142: 2560-2568, 1989.
Tedder, T. F.; Streuli, M.; Schlossman, S. F.; Saito, H.: Isolation and structure of a cDNA encoding the B1 (CD20) cell-surface antigen of human B lymphocytes. Proc. Nat. Acad. Sci. 85: 208-212, 1988.
Olavesen, M. G.; Bentley, E.; Mason, R. V. F.; Stephens, R. J.; Ragoussis, J.: Fine mapping of 39 ESTs on human chromosome 6p23-p25. Genomics 46:303-306, 1997
Thaung, C.; West, K.; Clark, B. J.; McKie, L.; Morgan, J. E.; Arnold, K.; Nolan, P. M.; Peters, J.; Hunter, A. J.; Brown, S. D. M.; Jackson, I. J.; Cross, S. H.: Novel ENU-induced eye mutations in the mouse: models for human eye disease. Hum. Molec. Genet. 11:755-767, 2002.
Baumann, C. A.; Ribon, V.; Kanzaki, M.; Thurmond, D. C.; Mora, S.; Shigematsu, S.; Bickel, P. E.; Pessin, J. E.; Saltiel, A. R.: CAP defines a second signalling pathway required for insulin-stimulated glucose transport. Nature 407: 202-207, 2000.
Brissenden, J. E.; Ullrich, A.; Francke, U.: Chromosomal mapping of loci for insulin-like growth factors I and II and for epidermal growth factor in man. (Abstract) Am. J. Hum. Genet. 36: 133S only, 1984.
Brissenden, J. E.; Ullrich, A.; Francke, U.: Human chromosomal mapping of genes for insulin-like growth factors I and II and epidermal growth factor. Nature 310: 781-784, 1984.
Morton, C. C.; Byers, M. G.; Nakai, H.; Bell, G. I.; Shows, T. B.: Human genes for insulin-like growth factors I and II and epidermal growth factor are located on 12q22-q24.1, 11p15, and 4q25-q27, respectively. Cytogenet. Cell Genet. 41: 245-249, 1986
Ishii, H.; Baffa, R.; Numata, S.-I.; Murakumo, Y.; Rattan, S.; Inoue, H.; Mori, M.; Fidanza, V.; Alder, H.; Croce, C. M.: The FEZ1 gene at chromosome 8p22 encodes a leucine-zipper protein, and its expression is altered in multiple human tumors. Proc. Nat. Acad. Sci. 96: 3928-3933, 1999.
Williamson, J. A.; Bosher, J. M.; Skinner, A.; Sheer, D.; Williams, T.; Hurst, H. C.: Chromosomal mapping of the human and mouse homologues of two new members of the AP-2 family of transcription factors. Genomics 35:262-264, 1996.
Agarwal, V. R.; Ashanullah, C. I.; Simpson, E. R.; Bulun, S. E.: Alternatively spliced transcripts of the aromatase cytochrome P450(CYP19) gene in adipose tissue of women. J. Clin. Endocr. Metab. 82:70-74, 1997.
Yang, R.; Morosetti, R.; Koeffler, H. P.: Characterization of a second human cyclin A that is highly expressed in testis and in several leukemic cell lines. Cancer Res. 57: 913-920, 1997.
Lew, D. J.; Dulic, V.; Reed, S. I.: Isolation of three novel human cyclins by rescue of G1 cyclin (cln) function in yeast. Cell 66:1197-1206, 1991.
Li, H.; Lahti, J. M.; Valentine, M.; Saito, M.; Reed, S. I.; Look, A. T.; Kidd, V. J.: Molecular cloning and chromosomal localization of the human cyclin C (CCNC) and cyclin E (CCNE) genes: deletion of the CCNC gene in human tumors. Genomics 32: 253-259, 1996.
Sheaff, R. J.; Groudine, M.; Gordon, M.; Roberts, J. M.; Clurman, B. E.: Cyclin E-CDK2 is a regulator of p27(Kip1). Genes Dev. 11:1464-1478, 1997.
Akoulitchev, S.; Chuikov, S.; Reinberg, D.: TFIIH is negatively regulated by cdk8-containing mediator complexes. Nature 407: 102-106, 2000.
Trask, B.; Fertitta, A.; Christensen, M.; Youngblom, J.; Bergmann, A.; Copeland, A.; de Jong, P.; Mohrenweiser, H.; Olsen, A.; Carrano, A.; Tynan, K.: Fluorescence in situ hybridization mapping of human chromosome 19: cytogenetic band location of 540 cosmids and 70 genes or DNA markers. Genomics 15: 133-145, 1993.
Holland, E. C.; Celestino, J.; Dai, C.; Schaefer, L.; Sawaya, R. E.; Fuller, G. N.: Combined activation of Ras and Akt in neural progenitors induces glioblastoma formation in mice. Nature Genet. 25:55-57, 2000.
Efstratiadis, A.; Posakony, J. W.; Maniatis, T.; Lawn, R. M.; O'Connell, C.; Spritz, R. A.; DeRiel, J. K.; Forget, B. G.; Weissman, S. M.; Slightom, J. L.; Blechl, A. E.; Smithies, O.; Baralle, F. E.; Shoulders, C. C.; Proudfoot, N. J.: The structure and evolution of the human beta-globin gene family. Cell 21: 653-668, 1980.
Doyle, J.; Hoffman, S.; Ucla, C.; Reith, W.; Mach, B.; Stubbs, L.: Locations of human and mouse genes encoding the RFX1 and RFX2 transcription factor proteins. Genomics 35: 227-230, 1996.
Pugliatti, L.; Derre, J.; Berger, R.; Ucla, C.; Reith, W.; Mach, B.: The genes for MHC class II regulatory factors RFX1 and RFX2 are located on the short arm of chromosome 19. Genomics 13: 1307-1310, 1992.
Reith, W.; Ucla, C.; Barras, E.; Gaud, A.; Durand, B.; Herrero-Sanchez, C.; Kobr, M.; Mach, B.: RFX1, a transactivator of hepatitis B virus enhancer 1, belongs to a novel family of homodimeric and heterodimeric DNA-binding proteins. Molec. Cell. Biol. 14: 1230-1244, 1994.
Creutz, C. E.; Tomsig, J. L.; Snyder, S. L.; Gautier, M.-C.; Skouri, F.; Beisson, J.; Cohen, J.: The copines, a novel class of C2 domain-containing, calcium-dependent, phospholipid-binding proteins conserved from Paramecium to humans. J. Biol. Chem. 273: 1393-1402, 1998.
Caudell, E. G.; Caudell, J. J.; Tang, C.-H.; Yu, T.-K.; Frederick, M. J.; Grimm, E. A.: Characterization of human copine III as a phosphoprotein with associated kinase activity. Biochemistry 39: 13034-13043, 2000.
Lai, C.-H.; Chou, C.-Y.; Ch'ang, L.-Y.; Liu, C.-S.; Lin, W.: Identification of novel human genes evolutionarily conserved in Caenorhabditis elegans by comparative proteomics. Genome Res. 10: 703-713, 2000.
Pappu, R.; Cheng, A. M.; Li, B.; Gong, Q.; Chiu, C.; Griffin, N.; White, M.; Sleckman, B. P.; Chan, A. C.: Requirement for B cell linker protein (BLNK) in B cell development. Science 286: 1949-1954, 1999.
Wienands, J.; Schweikert, J.; Wollscheid, B.; Jumaa, H.; Nielsen, P. J.; Reth, M.: SLP-65: a new signaling component in B lymphocytes which requires expression of the antigen receptor for phosphorylation. J. Exp. Med. 188: 791-795, 1998.
Muto, T.; Muramatsu, M.; Taniwaki, M.; Kinoshita, K.; Honjo, T.: Isolation, tissue distribution, and chromosomal localization of the human activation-induced cytidine deaminase (AID) gene. Genomics 68:85-88, 2000.
Geck, P.; Maffini, M. V.; Szelei, J.; Sonnenschein, C.; Soto, A. M.: Androgen-induced proliferative quiescence in prostate cancer cells: the role of AS3 as its mediator. Proc. Nat. Acad. Sci. 97:10185-10190, 2000.
Kas, K.; Finger, E.; Grall, F.; Gu, X.; Akbarali, Y.; Boltax, J.; Weiss, A.; Oettgen, P.; Kapeller, R.; Libermann, T. A.: ESE-3, a novel member of an epithelium-specific Ets transcription factor sub family, demonstrates different target gene specificity from ESE-1. J. Biol. Chem. 275: 2986-2998, 2000.
Kleinbaum, L. A.; Duggan, C.; Ferreira, E.; Coffey, G. P.; Buttice, G.; Burton, F. H.: Human chromosomal localization, tissue/tumor expression, and regulatory function of the ets family gene EHF. Biochem. Biophys. Res. Commun. 264: 119-126, 1999.
Zhao, L.; Gregoire, F.; Sul, H. S.: Transient induction of ENC-1, a kelch-related actin-binding protein, is required for adipocyte differentiation. J. Biol. Chem. 275: 16845-16850, 2000.
Scott, A. F.: Personal Communication. Baltimore, Md. Oct. 23, 2001.
Woodroofe, M. N.; Tunnacliffe, A.; Pym, B.; Goodfellow, P. N.; Walsh, F. S.: Human muscle cell surface antigen 16-3A5 is encoded by a gene on chromosome 11. Somat. Cell Molec. Genet. 10: 535-540, 1984.
Bilezikian, J. P.; Morishima, A.; Bell, J.; Grumbach, M. M.: Increased bone mass as a result of estrogen therapy in a man with aromatase deficiency. New Eng. J. Med. 339: 599-603, 1998.
Bulun, S. E.: Aromatase deficiency in women and men: would you have predicted the phenotypes? J. Clin. Endocr. Metab. 81: 867-871, 1996.
Carani, C.; Qin, K.; Simoni, M.; Faustini-Fustini, M.; Serpente, S.; Boyd, J.; Korach, K. S.; Simpson, E. R.: Effect of testosterone and estradiol in a man with aromatase deficiency. New Eng. J. Med. 337:91-95, 1997.
Chen, S.; Besman, M. J.; Sparkes, R. S.; Zollman, S.; Klisak, I.; Mohandas, T.; Hall, P. F.; Shively, J. E.: Human aromatase: cDNA cloning, Southern blot analysis, and assignment of the gene to chromosome 15. DNA 7: 27-38, 1988.
Chen, S.; Shively, J. E.; Nakajin, S.; Shinoda, M.; Hall, P. F.: Amino terminal sequence analysis of human placenta aromatase. Biochem. Biophys. Res. Commun. 135: 713-719, 1986.
Conte, F. A.; Grumbach, M. M.; Ito, Y.; Fisher, C. R.; Simpson, E. R.: A syndrome of female pseudohermaphrodism, hypergonadotropichypogonadism, and multicystic ovaries associated with missense mutations in the gene encoding aromatase (P450arom). J. Clin. Endocr. Metab. 78:1287-1292, 1994.
Corbin, C. J.; Graham-Lorence, S.; McPhaul, M.; Mason, J. I.; Mendelson, C. R.; Simpson, E. R.: Isolation of a full-length cDNA insert encoding human aromatase system cytochrome P-450 and its expression in nonsteroidogenic cells. Proc. Nat. Acad. Sci. 85: 8948-8952, 1988.
Deladoey, J.; Fluck, C.; Bex, M.; Yoshimura, N.; Harada, N.; Mullis, P. E.: Aromatase deficiency caused by a novel P450(arom) gene mutation: impact of absent estrogen production on serum gonadotropin concentration in a boy. J. Clin. Endocr. Metab. 84: 4050-4054, 1999.
Ellis, J. A.; Stebbing, M.; Harrap, S. B.: Significant population variation in adult male height associated with the Y chromosome and the aromatase gene. J. Clin. Endocr. Metab. 86: 4147-4150, 2001.
Evans, C. T.; Ledesma, D. B.; Schulz, T. Z.; Simpson, E. R.; Mendelson, C. R.: Isolation and characterization of a complementary DNA specific for human aromatase-system cytochrome P-450 mRNA. Proc. Nat. Acad. Sci. 83: 6387-6391, 1986.
Fisher, C. R.; Graves, K. H.; Parlow, A. F.; Simpson, E. R.: Characterization of mice deficient in aromatase (ArKO) because of targeted disruption of the cyp19 gene. Proc. Nat. Acad. Sci. 95:6965-6970, 1998.
George, F. W.; Matsumine, H.; McPhaul, M. J.; Somes, R. G., Jr.; Wilson, J. D.: Inheritance of the henny feathering trait in the Golden Campine chicken: evidence for allelism with the gene that causes henny feathering in the Sebright Bantam. J. Hered. 81: 107-110, 1990.
George, F. W.; Wilson, J. D.: Pathogenesis of the henny feathering trait in the Sebright Bantam chicken. J. Clin. Invest. 66: 57-65, 1980.
Harada, N.: Cloning of a complete cDNA encoding human aromatase: immunochemical identification and sequence analysis. Biochem. Biophys. Res. Commun. 156: 725-732, 1988.
Harada, N.; Ogawa, H.; Shozu, M.; Yamada, K.: Genetic studies to characterize the origin of the mutation in placental aromatase deficiency. Am. J. Hum. Genet. 51: 666-672, 1992.
Harada, N.; Ogawa, H.; Shozu, M.; Yamada, K.; Suhara, K.; Nishida, E.; Takagi, Y.: Biochemical and molecular genetic analyses on placental aromatase (P-450-AROM) deficiency. J. Biol. Chem. 267: 4781-4785, 1992.
Hemsell, D. L.; Edman, C. D.; Marks, J. F.; Siiteri, P. K.; MacDonald, P. C.: Massive extraglandular aromatization of plasma androstenedione resulting in feminization of a prepubertal boy. J. Clin. Invest. 60:455-464, 1977
Ito, Y.; Fisher, C. R.; Conte, F. A.; Grumbach, M. M.; Simpson, E. R.: Molecular basis of aromatase deficiency in an adult female with sexual infantilism and polycystic ovaries. Proc. Nat. Acad. Sci. 90: 11673-11677, 1993.
Leiberman, E.; Zachmann, M.: Familial adrenal feminization probably due to increased steroid aromatization. Hormone Res. 37: 96-102, 1992.
Leshin, M.; Baron, J.; George, F. W.; Wilson, J. D.: Increased estrogen formation and aromatase activity in fibroblasts cultured from the skin of chickens with the Henny feathering trait. J. Biol. Chem. 256: 4341-4344, 1981.
Leshin, M.; George, F. W.; Wilson, J. D.: Increased estrogen synthesis in the Sebright bantam is due to a mutation that causes increased aromatase activity. Trans. Assoc. Am. Phys. 94: 97-105, 1981.
Mango, D.; Montemurro, A.; Scirpa, P.; Bompiani, A.; Menini, E.: Four cases of pregnancy with low estrogen production due to placental enzymatic deficiency. Europ. J. Obstet. Gynec. Reprod. Biol. 8:65-71, 1978.
McTernan, P. G.; Anderson. L. A.; Anwar, A. J.; Eggo, M. C.; Crocker, J.; Barnett, A. H.; Stewart, P. M.; Kumar, S.: Glucocorticoid regulation of P450 aromatase activity in human adipose tissue: gender and site differences. J. Clin. Endocr. Metab. 87: 1327-1336, 2002.
Morishima, A.; Grumbach, M. M.; Simpson, E. R.; Fisher, C.; Qin, K.: Aromatase deficiency in male and female siblings caused by a novel mutation and the physiological role of estrogens. J. Clin. Endocr. Metab. 80: 3689-3698, 1995.
Mullis, P. E.; Yoshimura, N.; Kuhlmann, B.; Lippuner, K.; Jaeger, P.; Harada, H.: Aromatase deficiency in a female who is compound heterozygote for two new point mutations in the P450(arom) gene: impact of estrogens on hypergon adotropic hypogonadism, multicystic ovaries, and bone densitometry in childhood. J. Clin. Endocr. Metab. 82:1739-1745, 1997.
Phornphutkul, C.; Okubo, T.; Wu, K.; Harel, Z.; Tracy, T. F., Jr.; Pinar, H.; Chen, S.; Gruppuso, P. A.; Goodwin, G.: Aromatase P450 expression in a feminizing adrenal adenoma presenting as isosexual precocious puberty. J. Clin. Endocr. Metab. 86: 649-652, 2001.
Robertson, K. M.; O'Donnell, L.; Jones, M. E. E.; Meachem, S. J.; Boon, W. C.; Fisher, C. R.; Graves, K. H.; McLachlan, R. I.; Simpson, E. R.: Impairment of spermatogenesis in mice lacking a functional aromatase (cyp 19) gene. Proc. Nat. Acad. Sci. 96: 7986-7991, 1999
Sebastian, S.; Bulun, S. E.: A highly complex organization of the regulatory region of the human CYP19 (aromatase) gene revealed by the Human Genome Project. J. Clin. Endocr. Metab. 86: 4600-4602, 2001.
Everman, D. B.; Bartels, C. F.; Yang, Y.; Yanamandra, N.; Goodman, F. R.; Mendoza-Londono, J. R.; Savarirayan, R.; White, S. M.; Graham, J. M., Jr.; Gale, R. P.; Svarch, E.; Newman, W. G.; Kleckers, A. R.; Francomano, C. A.; Govindaiah, V.; Singh, L.; Morrison, S.; Thomas, J. T.; Warman, M. L.: The mutational spectrum of brachydactyly type C. Am. J. Med. Genet. 112: 291-296, 2002.
Galjaard, R. J. H.; van der Ham, L. I.; Posch, N. A. S.; Dijkstra, P. F.; Oostra, B. A.; Hovius, S. E. R.; Timmenga, E. J. F.; Sonneveld, G. J.; Hoogeboom, A. J. M.; Heutink, P.: Differences in complexity of isolated brachydactyly type C cannot be attributed to locus heterogeneity alone. Am. J. Med. Genet. 98: 256-262, 2001.
Haws, D. V.: Inherited brachydactyly and hypoplasia of the bones of the extremities. Ann. Hum. Genet. 26: 201-212, 1963.
Polinkovsky, A.; Robin, N. H.; Thomas, J. T.; Irons, M.; Lynn, A.; Goodman, F. R.; Reardon, W.; Kant, S. G.; Brunner, H. G.; vander Burgt, I.; Chitayat, D.; McGaughran, J.; Donnai, D.; Luyten, F. P.; Warman, M. L.: Mutations in CDMP1 cause autosomal dominant brachydactyly type C. (Letter) Nature Genet. 17: 18-19, 1997.
Polymeropoulos, M. H.; Ide, S. E.; Magyari, T.; Francomano, C. A.: Brachydactyly type C gene maps to human chromosome 12q24. Genomics 38:45-50, 1996.
Robin, N. H.; Gunay-Aygun, M.; Polinkovsky, A.; Warman, M. L.; Morrison, S.: Clinical and locus heterogeneity in brachydactyly type C. Am. J. Med. Genet. 68: 369-377, 1997.
Storm, E. E.; Huynh, T. V.; Copeland, N. G.; Jenkins, N. A.; Kingsley, D. M.; Lee, S. J.: Limb alterations in brachypodism mice due to mutations in a new member of the TGF-beta super family. Nature 368: 639-643, 1994.
Zetterstrom, R. H.; Solomin, L.; Jansson, L.; Hoffer, B. J.; Olson, L.; Perlmann, T.: Dopamine neuron agenesis in Nurr1-deficient mice. Science 276:248-250, 1997.
Koi, M.; Johnson, L. A.; Kalikin, L. M.; Little, P. F. R.; Nakamura, Y.; Feinberg, A. P.: Tumor cell growth arrest caused by subchromosomal transferable DNA fragments from chromosome 11. Science 260: 361-364, 1993.
Tsavaler, L.; Shapero, M. H.; Morkowski, S.; Laus, R.: Trp-p8, a novel prostate-specific gene, is up-regulated in prostate cancer and other malignancies and shares high homology with transient receptor potential calcium channel proteins. Cancer Res. 61: 3760-3769, 2001.
Hu, R.-J.; Lee, M. P.; Connors, T. D.; Johnson, L. A.; Burn, T. C.; Su, K.; Landes, G. M.; Feinberg, A. P.: A 2.5-Mb transcript map of a tumor-suppressing subchromosomal transferable fragment from 11p15.5, and isolation and sequence analysis of three novel genes. Genomics 46:9-17, 1997.
Schmidt, A.; Wolde, M.; Thiele, C.; Fest, W.; Kratzin, H.; Podtelejnikov, A. V.; Witke, W.; Huttner, W. B.; Soling, H.-D.: Endophilin I mediates synaptic vesicle formation by transfer of arachidonate to lysophosphatidic acid. Nature 401: 133-141, 1999.
Nagase, T.; Ishikawa, K.; Suyama, M.; Kikuno, R.; Hirosawa, M.; Miyajima, N.; Tanaka, A.; Kotani, H.; Nomura, N.; Ohara, O.: Prediction of the coding sequences of unidentified human genes. XII. The complete sequences of 100 new cDNA clones from brain which code for large proteins in vitro. DNA Res. 5: 355-364, 1998.
Dent, A. L.; Yewdell, J.; Puvion-Dutilleul, F.; Koken, M. H.; de The, H.; Staudt, L. M.: LYSP100 associated nuclear domains (LANDs): description of a new class of subnuclear structures and their relationship to PML nuclear bodies. Blood 88: 1423-1426, 1996.
Seeler, J. S.; Marchio, A.; Sitterlin, D.; Transy, C.; Dejean, A.: Interaction of SP100 with HP1 proteins: a link between the promyelocytic leukemia-associated nuclear bodies and the chromatin compartment. Proc. Nat. Acad. Sci. 95: 7316-7321, 1998.
Bahary, N.; Zorich, G.; Pachter, J. E.; Leibel, R. L.; Friedman, J. M.: Molecular genetic linkage maps of mouse chromosomes 4 and 6. Genomics 11: 33-47, 1991.
Callen, D. F.; Chen, L. Z.; Nancarrow, J.; Whitmore, S. A.; Apostolou, S.; Thompson, A. D.; Lane, S. A.; Stallings, R. L.; Hildebrand, C. E.; Harris, P. G.; Sutherland, G. R.: Current state of the physical map of human chromosome 16. (Abstract) Cytogenet. Cell Genet. 58:1998 only, 1991.
Corbi, A. L.; Larson, R. S.; Kishimoto, T. K.; Springer, T. A.; Morton, C. C.: Chromosomal location of the genes encoding the leukocyte adhesion receptors LFA-1, Mac-1 and p150,95: identification of a gene cluster involved in cell adhesion. J. Exp. Med. 167: 1597-1607, 1988.
Amagai, M.; Klaus-Kovtun, V.; Stanley, J. R.: Autoantibodies against a novel epithelial cadherin in pemphigus vulgaris, a disease of cell adhesion. Cell 67: 869-877, 1991
Koch, P. J.; Mahoney, M. G.; Ishikawa, H.; Pulkkinen, L.; Uitto, J.; Schultz, L.; Murphy, G. F.; Whitaker-Menezes, D.; Stanley, J. R.: Targeted disruption of the pemphigus vulgaris antigen (desmoglein3) gene in mice causes loss of keratinocyte cell adhesion with a phenotype similar to pemphigus vulgaris. J. Cell Biol. 137: 1091-1102, 1997.
Wang, Y.; Amagai, M.; Minoshima, S.; Sakai, K.; Green, K. J.; Nishikawa, T.; Shimizu, N.: The human genes for desmogleins (DSG1 and DSG3) are located in a small region on chromosome 18q12. Genomics 20:492-495, 1994.
DerKinderen, D. J.; Koten, J. W.; Tan, K. E. W. P.; Beemer, F. A.; Van Romunde, L. K. J.; Den Otter, W.: Parental age in sporadic hereditary retinoblastoma. Am. J. Ophthal. 110: 605-609, 1990.
Friedman, L. S.; Ostermeyer, E. A.; Lynch, E. D.; Welcsh, P.; Szabo, C. I.; Meza, J. E.; Anderson, L. A.; Dowd, P.; Lee, M. K.; Rowell, S. E.; Ellison, J.; Boyd, J.; King, M.-C.: 22 genes from chromosome 17q21: cloning, sequencing, and characterization of mutations in breast cancer families and tumors. Genomics 25: 256-163, 1995.
Linial, M.; Miller, K.; Scheller, R. H.: VAT-1: an abundant membrane protein from Torpedo cholinergic synaptic vesicles. Neuron 2: 1265-1273, 1989.
Smith, T. M.; Lee, M. K.; Szabo, C. I.; Jerome, N.; McEuen, M.; Taylor, M.; Hood, L.; King, M.-C.: Complete genomic sequence and analysis of 117 kb of human DNA containing the gene BRCA1. Genome Res. 6: 1029-1049, 1996.
Bause, E.; Bieberich, E.; Rolfs, A.; Volker, C.; Schmidt, B.: Molecular cloning and primary structure of Man(9)-mannosidase from human kidney. Eur. J. Biochem. 217: 535-540, 1993.
Tremblay, L. 0; Campbell Dyke, N.; Herscovics, A.: Molecular cloning, chromosomal mapping and tissue-specific expression of a novel human alpha-1,2-mannosidase gene involved in N-glycan maturation. Glycobiology 8:585-595, 1998.
Hubener, C.; Mincheva, A.; Lichter, P.; Schraven, B.; Bruyns, E.: Genomic organization and chromosomal localization of the human gene encoding the T-cell receptor interacting molecule (TRIM). Immunogenetics 51:154-158, 2000.
Bruyns, E.; Marie-Cardine, A.; Kirchgessner, H.; Sagolla, K.; Shevchenko, A.; Mann, M.; Autschbach, F.; Bensussan, A.; Meuer, S.; Schraven, B.: T cell receptor (TCR) interacting molecule (TRIM), a novel disulfide-linked dimer associated with the TCR-CD3-zeta complex, recruits intracellular signaling proteins to the plasma membrane. J. Exp. Med. 188: 561-575, 1998.
Zhao, R.; Qi, Y.; Chen, J.; Zhao, Z. J.: FYVE-DSP2, a FYVE domain-containing dual specificity protein phosphatase that dephosphorylates phosphotidylinositol(sic) 3-phosphate. Exp. Cell Res. 265: 329-338, 2001.
Frye, R. A.: Characterization of five human cDNAs with homology to the yeast SIR2 gene: Sir2-like proteins (sirtuins) metabolize NAD and may have protein ADP-ribosyltransferase activity. Biochem. Biophys. Res. Commun. 260: 273-279, 1999.
Imai, S.; Armstrong, C. M.; Kaeberlein, M.; Guarente, L.: Transcriptional silencing and longevity protein Sir2 is an NAD-dependent histone deacetylase. Nature 403:795-800, 2000.
Kimura, A.; Umehara, T.; Horikoshi, M.: Chromosomal gradient of histone acetylation established by Sas2p and Sir2p functions as a shield against gene silencing. Nature Genet. 15 Oct.: 2002. Note: Advance Electronic Publication.
Fedi, P.; Bafico, A.; Soria, A. N.; Burgess, W. H.; Miki, T.; Bottaro, D. P.; Kraus, M. H.; Aaronson, S. A.: Isolation and biochemical characterization of the human Dkk-1 homologue, a novel inhibitor of mammalian Wnt signaling. J. Biol. Chem. 274: 19465-19472, 1999.
Krupnik, V. E.; Sharp, J. D.; Jiang, C.; Robison, K.; Chickering, T. W.; Amaravadi, L.; Brown, D. E.; Guyot, D.; Mays, G.; Leiby, K.; Chang, B.; Duong, T.; Goodearl, A. D. J.; Gearing, D. P.; Sokol, S. Y.; McCarthy, S. A.: Functional and structural diversity of the human Dickkopf gene family. Gene 238: 301-313, 1999.
Roessler, E.; Du, Y.; Glinka, A.; Dutra, A.; Niehrs, C.; Muenke, M.: The genomic structure, chromosome location, and analysis of the human DKK1 head inducer gene as a candidate for holoprosencephaly. Cytogenet. Cell Genet. 89: 220-224, 2000.
Lebre, A.-S.; Jamot, L.; Takahashi, J.; Spasskey, N.; Leprince, C.; Ravise, N.; Zander, Fujigasaki, H.; Kussel-Andermann, P.; Duyckaerts, C.; Camonis, J. H.; Brice, A.: Ataxin-7 interacts with a Cbl-associated protein that it recruits into neuronal intranuclear inclusions. Hum. Molec. Genet. 10: 10:-1201-1213, 2001.
Lin, W.-H.; Chiu, K. C.; Chang, H.-M.; Lee, K.-C.; Tai, T.-Y.; Chuang, L.-M.: Molecular scanning of the human sorbin and SH3-domain-containing-1(SORBS1) gene: positive association of the T228A polymorphism with obesity and type 2 diabetes. Hum. Molec. Genet. 10: 1753-1760, 2001.
Scott, A. F.: Personal Communication. Baltimore, Md. Sep. 13, 2000.
Luo, J.; Nikolaev, A. Y.; Imai, S.; Chen, D.; Su, F.; Shiloh, A.; Guarente, L.; Gu, W.: Negative control of p53 by Sir2-alpha promotes cell survival under stress. Cell 107: 137-148, 2001.
Shore, D.; Squire, M.; Nasmyth, K. A.: Characterization of two genes required for the position-effect control of yeast mating-type genes. EMBO J. 3: 2817-2823, 1984.
Suka, N.; Luo, K.; Grunstein, M.: Sir2p and Sas2p opposingly regulate acetylation of yeast histone H4 lysine 16 and spreading of heterochromatin. Nature Genet. 15 Oct.: 2002. Note: Advance Electronic Publication.
Tanny, J. C.; Dowd, G. J.; Huang, J.; Hilz, H.; Moazed, D.: An enzymatic activity in the yeast Sir2 protein that is essential for gene silencing. Cell 99: 735-745, 1999.
Vaziri, H.; Dessain, S. K.; Eaton, E. N.; Imai, S.-I.; Frye, R. A.; Pandita, T. K.; Guarente, L.; Weinberg, R. A.: hSIR2—SIRT1 functions as an NAD-dependent p53 deacetylase. Cell 107: 149-159, 2001
Liu, J.; Shworak, N. W.; Sinay, P.; Schwartz, J. J.; Zhang, L.; Fritze, L. M.; Rosenberg, R. D.: Expression of heparan sulfate D-glucosaminyl 3-O-sulfotransferase isoforms reveals novel substrate specificities. J. Biol. Chem. 274: 5185-5192, 1999.
Shworak, N. W.; Liu, J.; Petros, L. M.; Zhang, L.; Kobayashi, M.; Copeland, N. G.; Jenkins, N. A.; Rosenberg, R. D.: Multiple isoforms of heparan sulfate D-glucosaminyl 3-O-sulfotransferase: isolation, characterization, and expression of human cDNAs and identification of distinct genomic loci. J. Biol. Chem. 274: 5170-5184, 1999.
Michel, J. J.; Xiong, Y.: Human CUL-1, but not other cullin family members, selectively interacts with SKP1 to form a complex with SKP2 and cyclin A. Cell Growth Differ. 9: 435-449, 1998.
Du, M.; Sansores-Garcia, L.; Zu, Z.; Wu, K. K.: Cloning and expression analysis of a novel salicylate suppressible gene, Hs-CUL-3, a member of cullin/Cdc53 family. J. Biol. Chem. 273: 24289-24292, 1998.
Bao, S.; Shen, X.; Shen, K.; Liu, Y.; Wang, X.-F.: The mammalian Rad24 homologous to yeast Saccharomyces cerevisiae Rad24 and Schizosaccharomyces pombe Rad17 is involved in DNA damage checkpoint. Cell Growth Diff. 9:961-967, 1998.
Bao, S.; Chang, M.-S.; Auclair, D.; Sun, Y.; Wang, Y.; Wong, W.-K.; Zhang, J.; Liu, Y.; Qian, X.; Sutherland, R.; Magi-Galluzi, C.; Weisberg, E.; Cheng, E. Y. S.; Hao, L.; Sasaki, H.; Campbell, M. S.; Kraeft, S.-K.; Loda, M.; Lo, K.-M.; Chen, L. B.: Hrad17, a human homologue of the Schizosaccharomyces pombe checkpoint gene rad17, is overexpressed in colon carcinoma. Cancer Res. 59: 2023-2028, 1999.
Bluyssen, H. A. R.; Naus, N. C.; van Os, R. I.; Jaspers, I.; Hoeijmakers, J. H. J.; de Klein, A.: Human and mouse homologues of the Schizosaccharomyces pombe rad17+ cell cycle checkpoint control gene. Genomics 55: 219-228, 1999.
Boyer, S. H.; Fainer, D. C.; Watson-Williams, E. J.: Lactate dehydrogenase variant from human blood: evidence for molecular subunits. Science 141:642-643, 1963.
Delmas, B.; Gelfi, J.; L'Haridon, R.; Vogel, L. K.; Sjostrom, H.; Noren, O.; Laude, H.: Aminopeptidase N is a major receptor for the enteropathogenic coronavirus TGEV. Nature 357: 417-420, 1992.
Kruse, T. A.; Bolund, L.; Grzeschik, K.-H.; Ropers, H. H.; Olsen, J.; Sjostrom, H.; Noren, O.: Assignment of the human aminopeptidase N (peptidase E) gene to chromosome 15q13-qter. FEBS Lett. 239: 305-308, 1988.
Look, A. T.; Ashmun, R. A.; Shapiro, L. H.; Peiper, S. C.: Human myeloid plasma membrane glyco protein CD13 (gp150) is identical to aminopeptidase N. J. Clin. Invest. 83: 1299-1307, 1989.
Look, A. T.; Peiper, S. C.; Rebentisch, M. B.; Ashmun, R. A.; Roussel, M. F.; Lemons, R. S.; Le Beau, M. M.; Rubin, C. M.; Sherr, C. J.: Molecular cloning, expression, and chromosomal localization of the gene encoding a human myeloid membrane antigen (gp150). J. Clin. Invest. 78: 914-921, 1986.
Watt, V. M.; Willard, H. F.: The human aminopeptidase N gene: isolation, chromosome localization, and DNA polymorphism analysis. Hum. Genet. 85: 651-654, 1990.
Yeager, C. L.; Ashmun, R. A.; Williams, R. K.; Cardellichio, C. B.; Shapiro, L. H.; Look, A. T.; Holmes, K. V.: Human aminopeptidase N is a receptor for human coronavirus 229E. Nature 357: 420-422, 1992.
Santoro, M.; Carlomagno, F.; Hay, I. D.; Herrmann, M. A.; Grieco, M.; Melillo, R.; Pierotti, M. A.; Bongarzone, I.; Della Porta, G.; Berger, N.; Peix, J. L.; Paulin, C.; Fabien, N.; Vecchio, G.; Jenkins, R. B.; Fusco, A.: Ret oncogene activation in human thyroid neoplasms is restricted to the papillary cancer subtype. J. Clin. Invest. 89:1517-1522, 1992.
Santoro, M.; Carlomagno, F.; Romano, A.; Bottaro, D. P.; Dathan, N. A.; Grieco, M.; Fusco, A.; Vecchio, G.; Matoskova, B.; Kraus, M. H.; Di Fiore, P. P.: Activation of RET as a dominant transforming gene by germline mutations of MEN2A and MEN2B. Science 267: 381-383, 1995.
Schuchardt, A.; D'Agati, V.; Larsson-Blomberg, L.; Costantini, F.; Pachnis, V.: Defects in the kidney and enteric nervous system of mice lacking the tyrosine kinase receptor Ret. Nature 367: 380-383, 1994.
Shirahama, S.; Ogura, K.; Takami, H.; Ito, K.; Tohsen, T.; Miyauchi, A.; Nakamura, Y.: Mutational analysis of the RET proto-oncogene in 71 Japanese patients with medullary thyroid carcinoma. J. Hum. Genet. 43:101-106, 1998.
Seri, M.; Yin, L.; Barone, A.; Bolino, A.; Celli, I.; Bocciardi, R.; Pasini, B.; Ceccherini, I.; Lerone, M.; Kristoffersson, U.; Larsson, L. T.; Casasa, J. M.; Cass, D. T.; Abramowicz, M. J.; Vanderwinden, J.-M.; Kravcenkiene, I.; Baric, I.; Silengo, M.; Martucciello, G.; Romeo, G.: Frequency of RET mutations in long- and short-segment Hirschsprung disease. Hum. Mutat. 9: 243-249, 1997.
Takahashi, M.; Buma, Y.; Hiai, H.: Isolation of ret proto-oncogene cDNA with an amino-terminal signal sequence. Oncogene 4: 805-806, 1989.
Takahashi, M.; Buma, Y.; Iwamoto, T.; Inaguma, Y.; Ikeda, H.; Hiai, H.: Cloning and expression of the ret protooncogene encoding a tyrosine kinase with two potential transmembrane domains. Oncogene 3:571-578, 1988.
Takahashi, M.; Ritz, J.; Cooper, G. M.: Activation of a novel human transforming gene, ret, by DNA rearrangement. Cell 42: 581-588, 1985.
Tessitore, A.; Sinisi, A. A.; Pasquali, D.; Cardone, M.; Vitale, D.; Bellastella, A.; Colantuoni, V.: A novel case of multiple endocrine neoplasia type 2A associated with two de novo mutations of the RET protooncogene. J. Clin. Endocr. Metab. 84: 3522-3527, 1999.
van Heyningen, V.: One gene—four syndromes. Nature 367: 319-320, 1994.
Xue, F.; Yu, H.; Maurer, L. H.; Memoli, V. A.; Nutile-McMenemey, N.; Schuster, M. K.; Bowden, D. W.; Mao, J.; Noll, W. W.: Germline RET mutations in MEN 2A and FMTC and their detection by simple DNA diagnostic tests. Hum. Molec. Genet. 3: 635-638, 1994.
Yin, L.; Ceccherini, I.; Pasini, B.; Matera, I.; Bicocchi, M. P.; Barone, V.; Bocciardi, R.; Kaariainen, H.; Weber, D.; Devoto, M.; Romeo, G.: Close linkage with the RET protooncogene and boundaries of deletion mutations in autosomal dominant Hirschsprung disease. Hum. Molec. Genet. 2: 1803-1808, 1993.
Berger, A.; Rosenthal, D.; Spiegel, S.: Sphingosylphosphocholine, a signaling molecule which accumulates in Niemann-Pick disease type A, stimulates DNA-binding activity of the transcription activator protein AP-1. Proc. Nat. Acad. Sci. 92: 5885-5889, 1995.
Bohmann, D.; Bos, T. J.; Admon, A.; Nishimura, T.; Vogt, P. K.; Tjian, R.: Human proto-oncogene c-jun encodes a DNA binding protein with structural and functional properties of transcription factor AP-1. Science 238: 1386-1392, 1987.
Bos, T. J.; Bohmann, D.; Tsuchie, H.; Tjian, R.; Vogt, P. K.: v-jun encodes a nuclear protein with enhancer binding properties of AP-1. Cell 52: 705-712, 1988.
Haluska, F. G.; Huebner, K.; Isobe, M.; Nishimura, T.; Croce, C. M.; Vogt, P. K.: Localization of the human JUN protooncogene to chromosome region 1p31-32. Proc. Nat. Acad. Sci. 85: 2215-2218, 1988.
Hattori, K.; Angel, P.; Le Beau, M. M.; Karin, M.: Structure and chromosomal localization of the functional intronless human JUN protooncogene. Proc. Nat. Acad. Sci. 85: 9148-9152, 1988.
Lamph, W. W.; Wamsley, P.; Sassone-Corsi, P.; Verma, I. M.: Induction of proto-oncogene JUN/AP-1 by serum and TPA. Nature 334: 629-631, 1988.
Marx, J. L.: ‘Jun’ is bustin' out all over. (Research News). Science 242:1377-1378, 1988.
Mattei, M. G.; Simon-Chazottes, D.; Hirai, S.-I.; Ryseck, R.-P.; Galcheva-Gargova, Z.; Guenet, J. L.; Mattei, J. F.; Bravo, R.; Yaniv, M.: Chromosomal localization of the three members of the jun proto-oncogene family in mouse and man. Oncogene 5: 151-156, 1990.
Shaulian, E.; Karin, M.: AP-1 as a regulator of cell life and-death. Nature Cell Biol. 4: E131-E136, 2002.
Shaulian, E.; Schreiber, M.; Piu, F.; Beeche, M.; Wagner, E. F.; Karin, M.: The mammalian UV response: c-Jun induction is required for exit from p53-imposed growth arrest. Cell 103: 897-907, 2000.
Whyte, J. R. C.; Munro, S.: The Sec34/35 Golgi transport complex is related to the exocyst, defining a family of complexes involved in multiple steps of membrane traffic. Dev. Cell 1: 527-537, 2001.
Hoefler, G.; Forstner, M.; McGuinness, M. C.; Hulla, W.; Hiden, M.; Krisper, P.; Kenner, L.; Ried, T.; Lengauer, C.; Zechner, R.; mOser, H. W.; Chen, G. L.: cDNA cloning of the human peroxisomalenoyl-CoA hydratase:3-hydroxyacyl-CoA dehydrogenase bifunctional enzyme and localization to chromosome 3q26.3-3q28: a free left Alu arm is inserted in the 3-prime noncoding region. Genomics 19: 60-67, 1994.
Nagle, D. L.; McGrail, S. H.; Vitale, J.; Woolf, E. A.; Dussault, B. J., Jr.; DiRocco, L.; Holmgren, L.; Montagno, J.; Bork, P.; Huszar, D.; Fairchild-Huntress, V.; Ge, P.; Keilty, J.; Ebelling, C.; Baldini, L.; Gilchrist, J.; Burr, P.; Carlson, G. A.; Moore, K. J.: The mahogany protein is a receptor involved in suppression of obesity. Nature 398:148-151, 1999.
Maas, S.; Gerber, A. P.; Rich, A.: Identification and characterization of a human tRNA-specific adenosine deaminase related to the ADAR family of pre-mRNA editing enzymes. Proc. Nat. Acad. Sci. 96: 8895-8900, 1999.
Zou, Y.-R.; Kottmann, A. H.; Kuroda, M.; Taniuchi, I.; Littman, D. R.: Function of the chemokine receptor CXCR4 in haematopoiesis and in cerebellar development. Nature 393: 595-599, 1998.
Apiou, F.; Flagiello, D.; Cillo, C.; Malfoy, B.; Poupon, M.-F.; Dutrillaux, B.: Fine mapping of human HOX gene clusters. Cytogenet. Cell Genet. 73: 114-115, 1996.
Brown, C. W.; Houston-Hawkins, D. E.; Woodruff, T. K.; Matzuk, M. M.: Insertion of Inhbb into the Inhba locus rescues the Inhba-null phenotype and reveals new activin functions. Nature Genet. 25: 453-457, 2000.
Ferguson, C. A.; Tucker, A. S.; Christensen, L.; Lau, A. L.; Matzuk, M. M.; Sharpe, P. T.: Activin is an essential early mesenchymal signal in tooth development that is required for patterning of the murine dentition. Genes Dev. 12: 2636-2649, 1998.
Burger, H. G.; Igarashi, M.; Baird, D.; Mason, T.; Bardin, W.; McLachlan, R.; Chappel, S.; Miyamoto, K.; de Jong, F.; Moudgal, A.; Demoulin, A.; Nieschlag, E.; de Kretser, D.; Robertson, D.; Findlay, J.; Sasamoto, S.; Forage, R.; Schwartz, N.; Fukuda, M.; Steinberger, A.; Hasegawa, Y.; Tanabe, K.; Ling, N.; Ying, S.-Y.: Inhibin: definition and nomenclature, including related substances. (Letter) J. Clin. Endocr. Metab. 66: 885-886, 1988.
Lumpkin, M. D.; Moltz, J. H.; Yu, W. H.; Samson, W. K.; McCann, S. M.: Purification of FSH-releasing factor: its dissimilarity from LHRH of mammalian, avian, and piscian origin. Brain Res. Bull. 18:175-178, 1987.
Matzuk, M. M.; Kumar, T. R.; Vassalli, A.; Bickenbach, J. R.; Roop, D. R.; Jaenisch, R.; Bradley, A.: Functional analysis of activins during mammalian development. Nature 374: 354-356, 1995.
Mellor, S. L.; Cranfield, M.; Ries, R.; Pedersen, J.; Cancilla, B.; de Kretser, D.; Groome, N. P.; Mason, A. J.; Risbridger, G. P.: Localization of activin beta(A)-, beta(B)-, and beta(C)-subunits in human prostate and evidence for formation of new activin heterodimers of beta(C)-subunit. J. Clin. Endocr. Metab. 85: 4851-4858, 2000.
Murata, M.; Eto, Y.; Shibai, H.; Sakai, M.; Muramatsu, M.: Erythroid differentiation factor is encoded by the same mRNA as that of the inhibin beta-A chain. Proc. Nat. Acad. Sci. 85: 2434-2438, 1988.
You, L.; Kruse, F. E.: Differential effect of activin A and BMP-7 on myofibroblast differentiation and the role of the Smad signaling pathway. Invest. Ophthal. Vis. Sci. 43: 72-81, 2002.
Dustin, M. L.; Olszowy, M. W.; Holdorf, A. D.; Li, J.; Bromley, S.; Desai, N.; Widder, P.; Rosenberger, F.; van der Merwe, P. A.; Allen, P. M.; Shaw, A. S.: A novel adaptor protein orchestrates receptor patterning and cytoskeletal polarity in T-cell contacts. Cell 94:667-677, 1998.
Kirsch, K. H.; Georgescu, M.-M.; Ishimaru, S.; Hanafusa, H.: CMS: an adapter molecule involved in cytoskeletal rearrangements. Proc. Nat. Acad. Sci. 96: 6211-6216, 1999.
Shih, N.-Y.; Li, J.; Karpitskii, V.; Nguyen, A.; Dustin, M. L.; Kanagawa, O.; Miner, J. H.; Shaw, A. S.: Congenital nephrotic syndrome in mice lacking CD2-associated protein. Science 286: 312-315, 1999.
Matsumoto-Taniura, N.; Pirollet, F.; Monroe, R.; Gerace, L.; Westendorf, J. M.: Identification of novel M phase phosphoproteins by expression cloning. Molec. Biol. Cell 7: 1455-1469, 1996.
Beckstead, R.; Ortiz, J. A.; Sanchez, C.; Prokopenko, S. N.; Chambon, P.; Losson, R.; Bellen, H. J.: Bonus, a Drosophila homolog of TIF1 proteins, interacts with nuclear receptors and can inhibit beta-FTZ-F1-dependent transcription. Molec. Cell 7: 753-765, 2001.
Le Douarin, B.; Zechel, C.; Garnier, J.-M.; Lutz, Y.; Tora, L.; Pierrat, B.; Heery, D.; Gronemeyer, H.; Chambon, P.; Losson, R.: The N-terminal part of TIF1, a putative mediator of the ligand-dependent activation function (AF-2) of nuclear receptors, is fused to B-rafin the oncogenic protein T18. EMBO J. 14: 2020-2033, 1995.
Thenot, S.; Henriquet, C.; Rochefort, H.; Cavailles, V.: Differential interaction of nuclear receptors with the putative human transcriptional coactivator hTIF1. J. Biol. Chem. 272: 12062-12068, 1997.
Feral, C.; Mattei, M. G.; Pawlak, A.; Guellaen, G.: Chromosomal localization of three human poly(A)-binding protein genes and four related pseudo genes. Hum. Genet. 105: 347-353, 1999.
Houng, A. K.; Maggini, L.; Clement, C. Y.; Reed, G. L.: Identification and structure of activated-platelet protein-1, a protein with RNA-binding domain motifs that is expressed by activated platelets. Europ. J. Biochem. 243: 209-218, 1997.
Yang, H.; Duckett, C. S.; Lindsten, T.: iPABP, an inducible poly(A)-binding protein detected in activated human T cells. Molec. Cell. Biol. 15:6770-6776, 1995.
Blagitko, N.; Schulz, U.; Schinzel, A. A.; Ropers, H.-H.; Kalscheuer, V. M.: Gamma-2-COP, a novel imprinted gene on chromosome 7q32, defines a new imprinting cluster in the human genome. Hum. Molec. Genet. 8:2387-2396, 1999.
Taylor, S. S.; Ha, E.; McKeon, F.: The human homologue of Bub3 is required for kinetochore localization of Bub1 and a Mad3/Bub1-related protein kinase. J. Cell Biol. 142: 1-11, 1998
Bergstein, I.; Eisenberg, L. M.; Bhalerao, J.; Jenkins, N. A.; Copeland, N. G.; Osborne, M. P.; Bowcock, A. M.; Brown, A. M. C.: Isolation of two novel WNT genes, WNT14 and WNT15, one of which (WNT15) is closely linked to WNT3 on human chromosome 17q21. Genomics 46:450-458, 1997.
Hartmann, C.; Tabin, C. J.: Wnt-14 plays a pivotal role in inducing synovial joint formation in the developing appendicular skeleton. Cell 104:341-351, 2001.
Saitoh, T.; Hirai, M.; Katoh, M.: Molecular cloning and characterization of WNT3A and WNT14 clustered in human chromosome 1q42 region. Biochem. Biophys. Res. Commun. 284: 1168-1175, 2001.
Kullak-Ublick, G.-A.; Beuers, U.; Meier, P. J.; Domdey, H.; Paumgartner, G.: Assignment of the human organic anion transporting polypeptide (OATP) gene to chromosome 12p12 by fluorescence in situ hybridization. J. Hepatol. 25: 985-987, 1996.
Kullak-Ublick, G. A.; Hagenbuch, B.; Stieger, B.; Schteingart, C. D.; Hofmann, A. F.; Wolkoff, A. W.; Meier, P. J.: Molecular and functional characterization of an organic anion transporting polypeptide cloned from human liver.: Gastroenterology 109: 1274-1282, 1995
Zheng, B.; Larkin, D. W.; Albrecht, U.; Sun, Z. S.; Sage, M.; Eichele, G.; Lee, C. C.; Bradley, A.: The mPer2 gene encodes a functional component of the mammalian circadian clock. Nature 400: 169-173, 1999.
Nakamura, H.; Sudo, T.; Tsuiki, H.; Miyake, H.; Morisaki, T.; Sasaki, J.; Masuko, N.; Kochi, M.; Ushio, Y.; Saya, H.: Identification of a novel human homolog of the Drosophila dlg, P-dlg, specifically expressed in the gland tissues and interacting with p55. FEBS Lett. 433: 63-67, 1998.
Funderburgh, J. L.; Perchellet, A. L.; Swiergiel, J.; Conrad, G. W.; Justice, M. J.: Keratocan (Kera), a corneal keratan sulfate proteoglycan, maps to the distal end of mouse chromosome 10. Genomics 52: 110-111, 1998.
Liu, C.-Y.; Shiraishi, A.; Kao, C. W.-C.; Converse, R. L.; Funderburgh, J. L.; Corpuz, L. M.; Conrad, G. W.; Kao, W. W.-Y.: The cloning of mouse keratocan cDNA and genomic DNA and the characterization of its expression during eye development. J. Biol. Chem. 273: 22584-22588, 1998.
Tasheva, E. S.; Funderburgh, J. L.; Funderburgh, M. L.; Corpuz, L. M.; Conrad, G. W.: Structure and sequence of the gene encoding human keratocan. DNA Seq. 10: 67-74, 1999.
Tasheva, E. S.; Pettenati, M.; Von Kap-Her, C.; Conrad, G. W.: Assignment of keratocan gene (KERA) to human chromosome band 12q22 by in situ hybridization. Cytogenet. Cell Genet. 88: 244-245, 2000.
Duncan, L. M.; Deeds, J.; Hunter, J.; Shao, J.; Holmgren, L. M.; Woolf, E. A.; Tepper, R. I.; Shyjan, A. W.: Downregulation of the novel gene melastatin correlates with potential for melanoma metastasis. Cancer Res. 58: 1515-1520, 1998.
Fang, D.; Setaluri, V.: Expression and up-regulation of alternatively spliced transcripts of melastatin, a melanoma metastasis-related gene, in human melanoma cells. Biochem. Biophys. Res. Commun. 279: 53-61, 2000.
Hunter, J. J.; Shao, J.; Smutko, J. S.; Dussault, B. J.; Nagle, D. L.; Woolf, E. A.; Holmgren, L. M.; Moore, K. J.; Shyjan, A. W.: Chromosomal localization and genomic characterization of the mouse melastatin gene (Mlsn1). Genomics 54: 116-123, 1998.
Xu, X. Z.; Moebius, F.; Gill, D. L.; Montell, C.: Regulation of melastatin, a TRP-related protein, through interaction with a cytoplasmic isoform. Proc. Nat. Acad. Sci. 98: 10692-10697, 2001.
Kalitsis, P.; Earle, E.; Fowler, K. J.; Choo, K. H. A.: Bub3 gene disruption in mice reveals essential mitotic spindle checkpoint function during early embryogenesis. Genes Dev. 14: 2277-2282, 2000.
van Hille, B.; Richener, H.; Evans, D. B.; Green, J. R.; Bilbe, G.: Identification of two subunit A isoforms of the vacuolar H(+)-ATPase in human osteoclastoma. J. Biol. Chem. 268: 7075-7080, 1993.
van Hille, B.; Richener, H.; Green, J. R.; Bilbe, G.: The ubiquitous VA68 isoform of subunit A of the vacuolar H(+)-ATPase is highly expressed in human osteoclasts. Biochem. Biophys. Res. Commun. 214: 1108-1113, 1995.
Liang, T. W.; Chiu, H. H.; Gurney, A.; Sidle, A.; Tumas, D. B.; Schow, P.; Foster, J.; Klassen, T.; Dennis, K.; DeMarco, R. A.; Pham, T.; Frantz, G.; Fong, S.: Vascular endothelial-junctional adhesion molecule (VE-JAM)/JAM 2 interacts with T, NK, and dendritic cells through JAM 3. J. Immun. 168: 1618-1626, 2002.
Arrate, M. P.; Rodriguez, J. M.; Tran, T. T.; Brock, T. A.; Cunningham, S. A.: Cloning of human junctional adhesion molecule 3 (JAM3) and its identification as the JAM2 counter-receptor. J. Biol. Chem. 276:45826-45832, 2001.
Parker, A. E.; Van de Weyer, I.; Laus, M. C.; Verhasselt, P.; Luyten, W. H. M. L.: Identification of a human homologue of the Schizosaccharomyces pombe rad17+ checkpoint gene. J. Biol. Chem. 273: 18340-18346, 1998. Note: Erratum: J. Biol. Chem. 274: 24438-24439, 1999.
von Deimling, F.; Scharf, J. M.; Liehr, T.; Rothe, M.; Kelter, A.-R.; Albers, P.; Dietrich, W. F.; Kunkel, L. M.; Wernert, N.; Wirth, B.: Human and mouse RAD17 genes: identification, localization, genomic structure and histological expression pattern in normal testis and seminoma. Hum. Genet. 105: 17-27, 1999.
Thomas, M. K.; Yao, K.-M.; Tenser, M. S.; Wong, G. G.; Habener, J. F.: Bridge-1, a novel PDZ-domain coactivator of E2A-mediated regulation of insulin gene transcription. Molec. Cell. Biol. 19: 8492-8504, 1999.
Watanabe, T. K.; Saito, A.; Suzuki, M.; Fujiwara, T.; Takahashi, E.; Slaughter, C. A.; DeMartino, G. N.; Hendil, K. B.; Chung, C. H.; Tanahashi, N.; Tanaka, K.: cDNA cloning and characterization of a human proteasomal modulator subunit, p27 (PSMD9). Genomics 50: 241-250, 1998.
Fu, C.; Turck, C. W.; Kurosaki, T.; Chan, A. C.: BLNK: a central linker protein in B cell activation. Immunity 9: 93-103, 1998.
Ishiai, M.; Kurosaki, M.; Pappu, R.; Okawa, K.; Ronko, I.; Fu, C.; Shibata, M.; Iwamatsu, A.; Chan, A. C.; Kurosaki, T.: BLNK required for cloning Syk to PLC-gamma-2 and Rac1-JNK in B cells. Immunity 10:117-125, 1999.
Minegishi, Y.; Rohrer, J.; Coustan-Smith, E.; Lederman, H. M.; Pappu, R.; Campana, D.; Chan, A. C.; Conley, M. E.: An essential role for BLNK in human B cell development. Science 286: 1954-1957, 1999.
DeVry, C. G.; Clarke, S.: Assignment of the protein L-isoaspartate (D-aspartate) O-methyltransferase gene (PCMT1) to human chromosome bands 6q24-q25 with radiation hybrid mapping. Cytogenet. Cell Genet. 84:130-131, 1999.
Farrar, C.; Clarke, S.: Altered levels of S-adenosylmethionine and S-adenosylhomocysteine in the brains of L-isoaspartyl (D-aspartyl)O-methyltransferase-deficient mice. J. Biol. Chem. 277: 27856-27863, 2002.
Ingrosso, D.; Kagan, R. M.; Clarke, S.: Distinct C-terminal sequences of isozymes I and II of the human erythrocyte L-isoaspartyl/D-aspartyl protein methyltransferase. Biochem. Biophys. Res. Commun. 175: 351-358, 1991.
Kim, E.; Lowenson, J. D.; Clarke, S.; Young, S. G.: Phenotypic analysis of seizure-prone mice lacking L-isoaspartate (D-aspartate)O-methyltransferase. J. Biol. Chem. 274: 20671-20678, 1999
Lowenson, J. D.; Kim, E.; Young, S. G.; Clarke, S.: Limited accumulation of damaged proteins in L-isoaspartyl (D-aspartyl) O-methyltransferase-deficient mice. J. Biol. Chem. 276: 20695-20702, 2001.
MacLaren, D. C.; Kagan, R. M.; Clarke, S.: Alternative splicing of the human isoaspartyl protein carboxyl methyl-transferase leads to the generation of a C-terminal -RDEL sequence in isozyme II. Biochem. Biophys. Res. Commun. 185: 277-283, 1992.
MacLaren, D. C.; O'Connor, C. M.; Xia, Y.-R.; Mehrabian, M.; Klisak, I.; Sparkes, R. S.; Clarke, S.; Lusis, A. J.: The L-isoaspartyl/D-aspartyl protein methyltransferase gene (PCMT1) maps to human chromosome 6q22.3-6q24 and the syntenic region of mouse chromosome 10. Genomics 14: 852-856, 1992.
Ota, I. M.; Gilbert, J. M.; Clarke, S.: Two major isozymes of the protein D-aspartyl/L-isoaspartyl methyltransferase from human erythrocytes. Biochem. Biophys. Res. Commun. 151: 1136-1143, 1988.
Hunter, A. G. W.; Clifford, B.; Cox, D. M.: The characteristic physiognomy and tissue specific karyotype distribution in the Pallister-Killian syndrome. Clin. Genet. 28: 47-53, 1985
Astrom, A.-K.; Voz, M. L.; Kas, K.; Roijer, E.; Wedell, B.; Mandahl, N.; Van de Ven, W.; Mark, J.; Stenman, G.: Conserved mechanism of PLAG1 activation in salivary gland tumors with and with out chromosome 8q12 abnormalities: identification of SII as a new fusion partner gene. Cancer Res. 59: 918-923, 1999.
Singh, S.; Chao, L. Y.; Mishra, R.; Davies, J.; Saunders, G. F.: Missense mutation at the C-terminus of PAX6 negatively modulates homeo domain function. Hum. Molec. Genet. 10: 911-918, 2001.
Singh, S.; Mishra, R.; Arango, N. A.; Deng, J. M.; Behringer, R. R.; Saunders, G. F.: Iris hypoplasia in mice that lack the alternatively spliced Pax6(5a) isoform. Proc. Nat. Acad. Sci. 99: 6812-6815, 2002.
Singh, S.; Tang, H. K.; Lee, J.-Y.; Saunders, G. F.: Truncation mutations in the transactivation region of PAX6 result in dominant-negative mutants. J. Biol. Chem. 273: 21531-21541, 1998.
Ton, C. C. T.; Miwa, H.; Saunders, G. F.: Small eye (Sey): cloning and characterization of the murine homolog of the human aniridia gene. Genomics 13:251-256, 1992.
Walther, C.; Gruss, P.: Pax-6, a murine paired box gene, is expressed in the developing CNS. Development 113: 1435-1449, 1991.
Wawersik, S.; Maas, R. L.: Vertebrate eye development as modeled in Drosophila. Hum. Molec. Genet. 9: 917-925, 2000.
Lee, D. K.; George, S. R.; Cheng, R.; Nguyen, T.; Liu, Y.; Brown, M.; Lynch, K. R.; O'Dowd, B. F.: Identification of four novel human G protein-coupled receptors expressed in the brain. Molec. Brain Res. 86: 13-22, 2001.
Cikos, S.; Gregor, P.; Koppel, J.: Cloning of a novel biogenic amine receptor-like G protein-coupled receptor expressed in human brain. Biochim. Biophys. Acta 1521: 66-72, 2001.
Chen, C.-K.; Zhang, K.; Church-Kopish, J.; Huang, W.; Zhang, H.; Chen, Y.-J.; Frederick, J. M.; Baehr, W.: Characterization of human GRK7 as a potential cone opsin kinase. Molec. Vision 7: 305-313, 2001.
Weiss, E. R.; Ducceschi, M. H.; Horner, T. J.; Li, A.; Craft, C. M.; Osawa, S.: Species-specific differences in expression of G-protein-coupled receptor kinase (GRK) 7 and GRK1 in mammalian cone photo receptor cells: implications for cone cell phototransduction. J. Neurosci. 21: 9175-9184, 2001.
Borregaard, N.; Cowland, J. B.: Granules of the human neutrophilic polymorphonuclear leukocyte. Blood 89: 3503-3521, 1997.
Chang, K. S.; Schroeder, W.; Siciliano, M. J.; Thompson, L. H.; McCredie, K.; Beran, M.; Freireich, E. J.; Liang, J. C.; Trujillo, J. M.; Stass, S. A.: The localization of the human myeloperoxidase gene is in close proximity to the translocation breakpoint in acute promyelocytic leukemia. Leukemia 1: 458-462, 1987.
DeLeo, F. R.; Goedken, M.; McCormick, S. J.; Nauseef, W. M.: A novel form of hereditary myeloperoxidase deficiency linked to endoplasmic reticulum/proteasome degradation. J. Clin. Invest. 101: 2900-2909, 1998.
Eiserich, J. P.; Baldus, S.; Brennan, M.-L.; Ma, W.; Zhang, C.; Tousson, A.; Castro, L.; Lusis, A. J.; Nauseef, W. M.; White, C. R.; Freeman, B. A.: Myeloperoxidase, a leukocyte-derived vascular NO oxidase. Science 296: 2391-2394, 2002.
Inazawa, J.; Inoue, K.; Nishigaki, H.; Tsuda, S.; Taniwaki, M.; Misawa, S.; Abe, T.: Assignment of the human myeloperoxidase gene (MPO) to bands q21.3-q23 of chromosome 17. Cytogenet. Cell Genet. 50:135-136, 1989.
Johnson, K.; Gemperlein, I.; Hudson, S.; Shane, S.; Rovera, G.: Complete nucleotide sequence of the human myeloperoxidase gene. Nucleic Acids Res. 17: 7985-7986, 1989.
Kizaki, M.; Miller, C. W.; Selsted, M. E.; Koeffler, H. P.: Myeloperoxidase (MPO) gene mutation in hereditary MPO deficiency. Blood 83: 1935-1940, 1994.
Klebanoff, S. J.: Myeloperoxidase. Proc. Assoc. Am. Physicians 111:383-389, 1999.
Kudoh, J.; Minoshima, S.; Hashinaka, K.; Nishio, C.; Yamada, M.; Shimizu, Y.; Shimizu, N.: Assignment of the myeloperoxidase gene MPO to human chromosome 17 using somatic cell hybrids and flow-sorted chromosomes. Jpn. J. Hum. Genet. 33: 315-324, 1988.
Kudoh, J.; Minoshima, S.; Hashinaka, K.; Nishio, C.; Yamada, M.; Shimizu, Y.; Shimizu, N.: Assignment of the myeloperoxidase (MPO) gene to human chromosome 17. (Abstract) Cytogenet. Cell Genet. 46:641-642, 1987.
Law, D. J.; Prasad, M. A.; King, S. E.; Spranger, K. D.; Lee, Y. H.; Fox, R. E.; Collins, E. E.; Gebuhr, T. C.; Miller, D. E.; Petty, E. M.: Localization of the human estrogen-responsive finger protein (EFP) gene (ZNF147) within a YAC contig containing the myeloperoxidase (MPO) gene. Genomics 28: 361-363, 1995.
Antonarakis, S. E.: Personal Communication. Baltimore, Md. Mar. 25, 2002
McKemy, D. D.; Neuhausser, W. M.; Julius, D.: Identification of a cold receptor reveals a general role for TRP channels in thermosensation. Nature 416:52-58, 2002.
Peier, A. M.; Moqrich, A.; Hergarden, A. C.; Reeve, A. J.; Andersson, D. A.; Story, G. M.; Earley, T. J.; Dragoni, I.; McIntyre, P.; Bevan, S.; Patapoutian, A.: A TRP channel that senses cold stimuli and menthol. Cell 108:705-715, 2002.
Ashery-Padan, R.; Marquardt, T.; Zhou, X.; Gruss, P.: Pax6 activity in the lens primordium is required for lens formation and for correct placement of a single retina in the eye. Genes Dev. 14: 2701-2711, 2000.
Azuma, N.; Yamaguchi, Y.; Handa, H.; Hayakawa, M.; Kanai, A.; Yamada, M.: Missense mutation in the alternative splice region of the PAX6 gene in eye anomalies. Am. J. Hum. Genet. 65: 656-663, 1999.
Roginski, R. S.; Mohan Raj, B. K.; Finkernagel, S. W.; Sciorra, L. J.: Assignment of an ionotropic glutamate receptor-like gene (GRINL1A) to human chromosome 15q22.1 by in situ hybridization. Cytogenet. Cell Genet. 93: 143-144, 2001.
Wydner, K. S.; Mohan Raj, B. K.; Sciorra, L. J.; Roginski, R. S.: The mouse orthologue of the human ionotropic glutamate receptor-like gene (GRINL1A) maps to mouse chromosome 9. Cytogenet. Cell Genet. 95:240-241, 2001.
Nakamura, T.; Yamazaki, Y.; Saiki, Y.; Moriyama, M.; Largaespada, D. A.; Jenkins, N. A.; Copeland, N. G.: Evi9 encodes a novel zinc-finger protein that physically interacts with BCL6, a known human B-cell proto-oncogene product. Molec. Cell Biol. 20: 3178-3186, 2000.
Saiki, Y.; Yamazaki, Y.; Yoshida, M.; Katoh, O.; Nakamura, T.: Human EVI9, a homologue of the mouse myeloid leukemia gene, is expressed in the hematopoietic progenitors and down-regulated during myeloid differentiation of HL60 cells. Genomics 70: 387-391, 2000.
Satterwhite, E.; Sonoki, T.; Willis, T. G.; Harder, L.; Nowak, R.; Arriola, E. L.; Liu, H.; Price, H. P.; Gesk, S.; Steinemann, D.; Schlegelberger, B.; Oscier, D. G.; Siebert, R.; Tucker, P. W.; Dyer, M. J. S.: The BCL11 gene family: involvement of BCL11A in lymphoid malignancies. Blood 98: 3413-3420, 2001.
Le Beau, M. M.; Lemons, R. S.; Rosner, G. L.; Carrino, J. C.; Reid, M. S.; Chisholm, R. L.; Diaz, M. O.; Weil, S. C.: Chromosomal localization of the gene encoding myeloperoxidase. (Abstract) Cytogenet. Cell Genet. 46: 645, 1987.
Richardson, J.; Cvekl, A.; Wistow, G.: Pax-6 is essential for lens-specific expression of zeta-crystallin. Proc. Nat. Acad. Sci. 92:4676-4680, 1995.
Sander, M.; Neubuser, A.; Kalamaras, J.; Ee, H. C.; Martin, G. R.; German, M. S.: Genetic analysis reveals that PAX6 is required for normal transcription of pancreatic hormone genes and islet development. Genes Dev. 11: 1662-1673, 1997.
Davis, A.; Cowell, J. K.: Mutations in the PAX6 gene in patients with hereditary aniridia. Hum. Molec. Genet. 2: 2093-2097, 1993.
Gronskov, K.; Rosenberg, T.; Sand, A.; Brondum-Nielsen, K.: Mutational analysis of PAX6: 16 novel mutations including 5 missense mutations with a mild aniridia phenotype. Europ. J. Hum. Genet. 7: 274-286, 1999.
Halder, G.; Callaerts, P.; Gehring, W. J.: Induction of ectopic eyes by targeted expression of the eyeless gene in Drosophila. Science 267:1788-1792, 1995.
Hanson, I.; Brown, A.; van Heyningen, V.: A new PAX6 mutationin familial aniridia. J. Med. Genet. 32: 488-489, 1995.
Hanson, I.; Van Heyningen, V.: Pax6: more than meets the eye. Trends Genet. 11: 268-272, 1995.
Hanson, I. M.; Fletcher, J. M.; Jordon, T.; Brown, A.; Taylor, D.; Adams, R. J.; Punnett, H. H.; van Heyningen, V.: Mutations at the PAX6 locus are found in heterogeneous anterior segment malformations including Peters' anomaly. Nature Genet. 6: 168-173, 1994.
Heins, N.; Malatesta, P.; Cecconi, F.; Nakafuku, M.; Tucker, K. L.; Hack, M. A.; Chapouton, P.; Barde, Y.-A.; Gotz, M.: Glial cells generate neurons: the role of the transcription factor Pax6. Nature Neurosci. 5: 308-315, 2002.
Holmstrom, G. E.; Reardon, W. P.; Baraitser, M.; Elston, J. S.; Taylor, D. S.: Heterogeneity in dominant anterior segment malformations. Brit. J. Ophthal. 75: 591-597, 1991.
Kioussi, C.; O'Connell, S.; St-Onge, L.; Treier, M.; Gleiberman, A. S.; Gruss, P.; Rosenfeld, M. G.: Pax6 is essential for establishing ventral-dorsal cell boundaries in pituitary gland development. Proc. Nat. Acad. Sci. 96: 14378-14382, 1999.
Kleinjan, D. A.; Seawright, A.; Schedl, A.; Quinlan, R. A.; Danes, S.; van Heyningen, V.: Aniridia-associated translocations, DNase hypersensitivity, sequence comparison and transgenic analysis redefine the functional domain of PAX6. Hum. Molec. Genet. 10: 2049-2059, 2001.
Lauderdale, J. D.; Wilensky, J. S.; Oliver, E. R.; Walton, D. S.; Glaser, T.: 3-prime deletions cause aniridia by preventing PAX6 gene expression. Proc. Nat. Acad. Sci. 97: 13755-13759, 2000.
Marquardt, T.; Ashery-Padan, R.; Andrejewski, N.; Scardigli, R.; Guillemot, F.; Gruss, P.: Pax6 is required for the multipotent state of retinal progenitor cells. Cell 105: 43-55, 2001.
Bentz, H.; Nathan, R. M.; Rosen, D. M.; Armstrong, R. M.; Thompson, A. Y.; Segarini, P. R.; Mathews, M. C.; Dasch, J. R.; Piez, K. A.; Seyedin, S. M.: Purification and characterization of a unique osteoinductive factor from bovine bone. J. Biol. Chem. 264: 20805-20810, 1989.
Madisen, L.; Neubauer, M.; Plowman, G.; Rosen, D.; Segarini, P.; Dasch, J.; Thompson, A.; Ziman, J.; Bentz, H.; Purchio, A. F.: Molecular cloning of a novel bone-forming compound: osteoinductive factor. DNA Cell Biol. 9: 303-309, 1990.
Chen, H.; Antonarakis, S. E.: The SH3D1A gene maps to human chromosome 21q22.1-q22.2. Cytogenet. Cell Genet. 78: 213-215, 1997.
Pucharcos, C.; Estivill, X.; de la Luna, S.: Intersectin 2, anew multimodular protein involved in clathrin-mediated endocytosis. FEBS Lett. 478: 43-51, 2000.
Gibbons, B.; Scott, D.; Hungerford, J. L.; Cheung, K. L.; Harrison, C.; Attard-Montalto, S.; Evans, M.; Birch, J. M.; Kingston, J. E.: Retinoblastoma in association with the chromosome breakage syndromes Fanconi's anaemia and Bloom's syndrome: clinical and cytogenetic findings. Clin. Genet. 47: 311-317, 1995.
Girardet, A.; McPeek, M. S.; Leeflang, E. P.; Munier, F.; Arnheim, N.; Claustres, M.; Pellestor, F.: Meiotic segregation analysis of RB1 alleles in retinoblastoma pedigrees by use of single-sperm typing. Am. J. Hum. Genet. 66: 167-175, 2000.
Godbout, R.; Dryja, T. P.; Squire, J.; Gallie, B. L.; Phillips, R. A.: Somatic inactivation of genes on chromosome 13 is a common event in retinoblastoma. Nature 304: 451-453, 1983.
Goodrich, D. W.; Wang, N. P.; Qian, Y.-W.; Lee, E. Y.-H. P.; Lee, W.-H.: The retinoblastoma gene product regulates progression through the G1 phase of the cell cycle. Cell 67: 293-302, 1991.
Grace, E.; Drennan, J.; Colver, D.; Gordon, R. R.: The 13q deletion syndrome. J. Med. Genet. 8: 351-357, 1971.
Green, A. R.; Wyke, J. A.: Anti-oncogenes: a subset of regulatory genes involved in carcinogenesis? Lancet II: 475-477, 1985.
Greger, V.; Kerst, S.; Messmer, E.; Hopping, W.; Passarge, E.; Horsthemke, B.: Application of linkage analysis to genetic counselling in families with hereditary retinoblastoma. J. Med. Genet. 25: 217-221, 1988.
Greger, V.; Passarge, E.; Horsthemke, B.: Somatic mosaicism in a patient with bilateral retinoblastoma. Am. J. Hum. Genet. 46:1187-1193, 1990.
Hagstrom, S. A.; Dryja, T. P.: Mitotic recombination map of 13cen-13q14 derived from an investigation of loss of heterozygosity in retinoblastomas. Proc. Nat. Acad. Sci. 96: 2952-2957, 1999.
Hall, J. G.: Personal Communication. Vancouver, British Columbia, Canada May 29, 1993.
Hanahan, D.; Weinberg, R. A.: The hallmarks of cancer. Cell 100:57-70, 2000.
Harbour, J. W.: Molecular basis of low-penetrance retinoblastoma. Arch. Ophthal. 119: 1699-1704, 2001.
Harbour, J. W.; Lai, S.-L.; Whang-Peng, J.; Gazdar, A. F.; Minna, J. D.; Kaye, F. J.: Abnormalities in structure and expression of the human retinoblastoma gene in SCLC. Science 241: 353-357, 1988.
Hensel, C.; Hsieh, C.-L.; Lee, W.-H.; Pam-Lee, E.; Gazdar, A.; Sakaguchi, A. Y.; Naylor, S. L.: Allele loss and lack of expression of the RB-1 locus in small cell lung cancer. (Abstract) Am. J. Hum. Genet. 43: A25, 1988.
Henson, J. W.; Schnitker, B. L.; Correa, K. M.; von Diemling, A.; Fassbender, F.; Xu, H.-J.; Benedict, W. F.; Yandell, D. W.; Louis, D. N.: The retinoblastoma gene is involved in malignant progression of astrocytomas. Ann. Neurol. 36: 714-721, 1994.
Higgins, M. J.; Hansen, M. F.; Cavenee, W. K.; Lalande, M.: Molecular detection of chromosomal translocations that disrupt the putative retinoblastoma susceptibility locus. Molec. Cell. Biol. 9: 1-5, 1989.
Hoegerman, S. F.: Chromosome 13 long arm interstitial deletion may result from maternal inverted insertion. Science 205: 1035-1036, 1979.
Hogg, A.; Bia, B.; Onadim, Z.; Cowell, J. K.: Molecular mechanisms of oncogenic mutations in tumours from patients with bilateral and unilateral retinoblastoma. Proc. Nat. Acad. Sci. 90: 7351-7355, 1993.
Honavar, S. G.; Shields, C. L.; Shields, J. A.; Demirci, H.; Naduvilath, T. J.: Intraocular surgery after treatment of retinoblastoma. Arch. Ophthal. 119: 1613-1621, 2001.
Honavar, S. G.; Singh, A. D.; Shields, C. L.; Meadows, A. T.; Demirci, H.; Cater, J.; Shields, J. A.: Postenucleation adjuvant therapy in high-risk retinoblastoma. Arch. Ophthal. 120: 923-931, 2002.
Hong, F. D.; Huang, H.-J. S.; To, H.; Young, L.-J. S.; Oro, A.; Bookstein, R.; Lee, E. Y.-H. P.; Lee, W.-H.: Structure of the human retinoblastoma gene. Proc. Nat. Acad. Sci. 86: 5502-5506, 1989.100. Horowitz, J. M.; Park, S.-H.; Bogenmann, E.; Cheng, J.-C.; Yandell, D. W.; Kaye, F. J.; Minna, J. D.; Dryja, T. P.; Weinberg, R. A.: Frequent inactivation of the retinoblastoma anti-oncogene is restricted to a subset of human tumor cells. Proc. Nat. Acad. Sci. 87: 2775-2779, 1990.101. Horowitz, J. M.; Park, S. H.; Yandell, D. W.; Weinberg, R. A.: Involvement of the retinoblastoma gene in the genesis of various human tumors:. In: Kavenee, W.; Hastie, N.; Stanbridge, E.: Recessive Oncogenes and Tumor Suppression: Current Communications in Molecular Biology. Cold Spring Harbor, N.Y.: Cold Spring Harbor Laboratory Press (pub.) 1989. Pp. 101-108.102. Horsthemke, B.; Greger, V.; Barnert, H. J.; Hopping, W.; Passarge, E.: Detection of submicroscopic deletions and a DNA polymorphism at the retinoblastoma locus. Hum. Genet. 76: 257-261, 1987.103. Horsthemke, B.; Greger, V.; Becher, R.; Passarge, E.: Mechanism of i(6p) formation in retinoblastoma tumor cells. Cancer Genet. Cytogenet. 37:95-102, 1989.104. Hsieh, J.-K.; Chan, F. S. G.; O'Connor, D. J.; Mittnacht, S.; Zhong, S.; Lu, X.: RB regulates the stability and the apoptotic function of p53 via MDM2. Molec. Cell 3: 181-193, 1999.105. Huang, H.-J. S.; Yee, J.-K.; Shew, J.-Y.; Chen, P.-L.; Bookstein, R.; Friedmann, T.; Lee, E. Y.-H. P.; Lee, W.-H.: Suppression of the neoplastic phenotype by replacement of the RB gene in human cancer cells. Science 242: 1563-1566, 1988.106.
Janson, M.; Nordenskjold, M.: A constitutional mutation within the retinoblastoma gene detected by PFGE. Clin. Genet. 45: 5-10, 1994.107. Jensen, R. D.; Miller, R. W.: Retinoblastoma: epidemiologic characteristics. New Eng. J. Med. 285: 307-311, 1971.108. Kimchi, A.; Wang, X.-F.; Weinberg, R. A.; Cheifetz, S.; Massague, J.: Absence of TGF-beta receptors and growth inhibitory responses in retinoblastoma cells. Science 240: 196-199, 1988.109.
Kitchin, F. D.; Ellsworth, R. M.: Pleiotropic effects of the gene for retinoblastoma. J. Med. Genet. 11: 244-246, 1974.110. Kivela, T.: Trilateral retinoblastoma: a meta-analysis of hereditary retinoblastoma associated with primary ectopic intracranial retinoblastoma. J. Clin. Oncol. 17: 1829-1837, 1999.111. Kivela, T.; Asko-Seljavaara, S.; Pihkala, U.; Hovi, L.; Heikkonen, J.: Sebaceous carcinoma of the eyelid associated with retinoblastoma. Ophthalmology 108:1124-1128, 2001.112. Klutz, M.; Brockmann, D.; Lohmann, D. R.: A parent-of-origin effect in two families with retinoblastoma is associated with a distinct splice mutation in the RB1 gene. Am. J. Hum. Genet. 71: 174-179, 2002.113. Knight, L. A.; Gardner, H. A.; Gallie, B. L.: Familial retinoblastoma: segregation of chromosome 13 in four families. Am. J. Hum. Genet. 32:194-201, 1980.114. Knudson, A. G.: Hereditary cancer, oncogenes and anti-oncogenes. Cancer Res. 45: 1437-1443, 1985.115. Knudson, A. G., Jr.: Mutation and cancer: statistical study of retinoblastoma. Proc. Nat. Acad. Sci. 68: 820-823, 1971.116. Knudson, A. G., Jr.: Genetics of human cancer. Annu. Rev. Genet. 20:231-251, 1986.117.
Knudson, A. G., Jr.; Hethcote, H. W.; Brown, B. W.: Mutation and childhood cancer: a probabilistic model for the incidence of retinoblastoma. Proc. Nat. Acad. Sci. 72: 5116-5120, 1975.118. Knudson, A. G., Jr.; Meadows, A. T.; Nichols, W. W.; Hill, R.: Chromosomal deletion and retinoblastoma. New Eng. J. Med. 295:1120-1123, 1976.119. Laquis, S. J.; Rodriguez-Galindo, C.; Wilson, M. W.; Fleming, J. C.; Haik, B. G.: Retinoblastoma in a patient with an X; 13 translocation and facial abnormalities consistent with 13q-syndrome. Am. J. Ophthal. 133:285-287, 2002.120. Lee, W.-H.; Bookstein, R.; Hong, F.; Young, L.-J.; Shew, J.-Y.; Lee, E. Y.-H. P.: Human retinoblastoma susceptibility gene: cloning, identification, and sequence. Science 235: 1394-1399, 1987.121. Lee, W.-H.; Shew, J.-Y.; Hong, F. D.; Sery, T. W.; Donoso, L. A.; Young, L.-J.; Bookstein, R.; Lee, E. Y.-H. P.: The retinoblastoma susceptibility gene encodes a nuclear phosphoprotein associated with DNA binding activity. Nature 329: 642-645, 1987.122. Lele, K. P.; Penrose, L. S.; Stallard, H. B.: Chromosome deletion in a case of retinoblastoma. Ann. Hum. Genet. 27: 171-174, 1963.123. Lemieux, N.; Messier, P. E.; Jacob, J. L.; Milot, J.; Richer, C. L.: Precise cytogenetic localization of the Rb locus at subband 13q14.11 by ultrastructural detection after immunochemical chromosome banding. (Abstract) Am. J. Hum. Genet. 45 (suppl.): A27, 1989.124. Liu, Z.; Song, Y.; Bia, B.; Cowell, J. K.: Germline mutations in the RB1 gene in patients with hereditary retinoblastoma. Genes Chromosomes Cancer 14: 277-284, 1995.125. Lohmann, D. R.; Brandt, B.; Hopping, W.; Passarge, E.; Horsthemke, B.: Spectrum of small length germline mutations in the RB1 gene. Hum. Molec. Genet. 3: 2187-2193, 1994.126. Lohmann, D. R.; Brandt, B.; Hopping, W.; Passarge, E.; Horsthemke, B.: The spectrum of RB1 germ-line mutations in hereditary retinoblastoma. Am. J. Hum. Genet. 58: 940-949, 1996.127. Lohmann, D. R.; Gerick, M.; Brandt, B.; Oelschlager, U.; Lorenz, B.; Passarge, E.; Horsthemke, B.: Constitutional RB1-gene mutations in patients with isolated unilateral retinoblastoma. Am. J. Hum. Genet. 61: 282-294, 1997.128. Lomazzi, M.; Moroni, M. C.; Jensen, M. R.; Frittoli, E.; Helin, K.: Suppression of the p53- or pRB-mediated G1 checkpoint is required for E2F-induced S-phase entry. Nature Genet 31: 190-194, 2002.129. Lueder, G. T.; Judisch, G. F.; Wen, B.-C.: Heritable retinoblastoma and pinealoma. Arch. Ophthal. 109: 1707-1709, 1991.130. Luo, R. X.; Postigo, A. A.; Dean, D. C.: Rb interacts with histone deacetylase to repress transcription. Cell 92: 463-473, 1998.131. Maat-Kievit, J. A.; Oepkes, D.; Hartwig, N. G.; Vermeij-Keers, C.; van Kamp, I. L.; van de Kamp, J. J. P.: A large retinoblastoma detected in a fetus at 21 weeks of gestation. Prenatal Diag. 13:377-384, 1993.132. MacKay, C. J.; Abramson, D. H.; Ellsworth, R. M.: Metastatic patterns of retinoblastoma. Arch. Ophthal. 102: 391-396, 1984.133. Macklin, M. T.: A study of retinoblastoma in Ohio. Am. J. Hum. Genet. 12: 1-43, 1960.134. Macklin, M. T.: Inheritance of retinoblastoma in Ohio. Arch. Ophthal. 62: 842-851, 1959.135
Manchester, P. T., Jr.: Retinoblastoma among offspring of adult survivors. Arch. Ophthal. 65: 546-549, 1961.136.
Mancini, D.; Singh, S.; Ainsworth, P.; Rodenhiser, D.: Constitutively methylated CpG dinucleotides as mutation hot spots in the retinoblastoma gene (RB1). Am. J. Hum. Genet. 61: 80-87, 1997.137. Mancini, M. A.; Shan, B.; Nickerson, J. A.; Penman, S.; Lee, W.-H.: The retinoblastoma gene product is a cell cycle-dependent, nuclear matrix-associated protein. Proc. Nat. Acad. Sci. 91: 418-422, 1994.138. Marino, S.; Vooijs, M.; van der Gulden, H.; Jonker, J.; Berns, A.: Induction of medulloblastomas in p53-null mutant mice by somatic inactivation of Rb in the external granular layer cells of the cerebellum. Genes Dev. 14: 994-1004, 2000.139. Matsunaga, E.: Recurrence risks to relatives of patients with retinoblastoma. Jpn. J. Ophthal. 22: 313-319, 1978.140. Matsunaga, E.: Almost synchronous appearance of bilateral retinoblastomas. (Letter) Am. J. Med. Genet. 11: 485-487, 1982.141.
Matsunaga, E.: Retinoblastoma: mutational mosaicism or host resistance? Am. J. Med. Genet. 8: 375-387, 1981.142.
Matsunaga, E.: Hereditary retinoblastoma: host resistance and second primary tumors. J. Nat. Cancer Inst. 65: 47-51, 1980.143. Matsunaga, E.: Hereditary retinoblastoma: delayed mutation or host resistance? Am. J. Hum. Genet. 30: 406-425, 1978.144. Matsunaga, E.; Minoda, K.; Sasaki, M. S.: Parental age and seasonal variation in the births of children with sporadic retinoblastoma: a mutation-epidemiologic study. Hum. Genet. 84: 155-158, 1990.145. Michalova, K.; Kloucek, F.; Musilova, J.: Deletion of 13q in two patients with retinoblastoma, one probably due to 13q-mosaicismin the mother. Hum. Genet. 61: 264-266, 1982.146. Moll, A. C.; Imhof, S. M.; Schouten-Van Meeteren, A. Y. N.; Kuik, D. J.; Hofman, P.; Boers, M.: Second primary tumors in hereditary retinoblastoma: a register-based study, 1945-1997. Is there an age effect on radiation-related risk? Ophthalmology 108: 1109-1114, 2001.147. Motegi, T.: Lymphocyte chromosome survey in 42 patients with retinoblastoma: effort to detect 13q14 deletion mosaicism. Hum. Genet. 58:168-173, 1981.148. Motegi, T.: High rate of detection of 13q14 deletion mosaicism among retinoblastoma patients (using more extensive methods). Hum. Genet. 61: 95-97, 1982.149. Motegi, T.; Kaga, M.; Yanagawa, Y.; Kadowaki, H.; Watanabe, K.; Inoue, A.; Komatsu, M.; Minoda, K.: A recognizable pattern of the midface of retinoblastoma patients with interstitial deletion of 13q. Hum. Genet. 64: 160-162, 1983.150. Motegi, T.; Komatsu, M.; Minoda, K.: Is the interstitial deletion of 13q in retinoblastoma patients not transmissible? (Letter) Hum. Genet. 64: 205, 1983.151. Motegi, T.; Komatsu, M.; Nakazato, Y.; Ohuchi, M.; Minoda, K.: Retinoblastoma in a boy with a de novo mutation of a 13/18 translocation: the assumption that the retinoblastoma locus is at 13q141, particularly at the distal portion of it. Hum. Genet. 60: 193-195, 1982.152. Munier, F.; Spence, M. A.; Pescia, G.; Balmer, A.; Gailloud, C.; Thonney, F.; van Melle, G.; Rutz, H. P.: Paternal selection favoring mutant alleles of the retinoblastoma susceptibility gene. Hum. Genet. 89:508-512, 1992.153. Munier, F. L.; Wang, M. X.; Spence, M. A.; Thonney, F.; Balmer, A.; Pescia, G.; Donoso, L. A.; Murphree, A. L.: Pseudo low penetrance in retinoblastoma: fortuitous familial aggregation of sporadic cases caused by independently derived mutations in two large pedigrees. Arch. Ophthal. 111: 1507-1511, 1993.154. Murphree, A. L.; Benedict, W. F.: Retinoblastoma: clues to human oncogenesis. Science 223: 1028-1033, 1984.155. Naumova, A.; Hansen, M.; Strong, L.; Jones, P. A.; Hadjistilianou, D.; Mastrangelo, D.; Griegel, S.; Rajewsky, M. F.; Shields, J.; Donoso, L.; Wang, M.; Sapienza, C.: Concordance between parental origin of chromosome 13q loss and chromosome 6p duplication in sporadic retinoblastoma. Am. J. Hum. Genet. 54: 274-281, 1994.156. Naumova, A.; Sapienza, C.: The genetics of retinoblastoma, revisited. Am. J. Hum. Genet. 54: 264-273, 1994.157. Nevins, J. R.: The Rb/E2F pathway and cancer. Hum. Molec. Genet. 10:699-703, 2001.158.
Nichols, W. W.; Miller, R. C.; Sobel, M.; Hoffman, E.; Sparkes, R. S.; Mohandas, T.; Veomett, I.; Davis, J. R.: Further observations on a 13qXp translocation associated with retinoblastoma. Am. J. Ophthal. 89:621-627, 1980.159.
Nielsen, S. J.; Schneider, R.; Bauer, U.-M.; Bannister, A. J.; Morrison, A.; O'Carroll, D.; Firestein, R.; Cleary, M.; Jenuwein, T.; Herrera, R. E.; Kouzarides, T.: Rb targets histone H3 methylation and HP1 to promoters. Nature 412: 561-565, 2001.160. Nirankari, M. S.; Gulati, G. C.; Chaddah, M. R.: Retinoblastoma: genetics and report of a family. Am. J. Ophthal. 53: 523-532, 1962.161. Noorani, H. Z.; Khan, H. N.; Gallie, B. L.; Detsky, A. S.: Cost comparison of molecular versus conventional screening of relatives at risk for retinoblastoma. Am. J. Hum. Genet. 59: 301-307, 1996.162. Nussbaum, R.; Puck, J.: Recurrence risks for retinoblastoma: a model for autosomal dominant disorders with complex inheritance. J. Pediat. Ophthal. 13: 89-98, 1976.163. Onadim, Z.; Hogg, A.; Baird, P. N.; Cowell, J. K.: Oncogenic point mutations in exon 20 of the RB1 gene in families showing incomplete penetrance and mild expression of the retinoblastoma phenotype. Proc. Nat. Acad. Sci. 89: 6177-6181, 1992.164. Onadim, Z.; Woolford, A. J.; Kingston, J. E.; Hungerford, J. L.: The RB1 gene mutation in a child with ectopic intracranial retinoblastoma. Brit. J. Cancer 76: 1405-1409, 1997.165. Ono, T.; Yoshida, M. C.: Chromosomal assignment of retinoblastomal gene (RB1) to mouse 14D3 and rat 15q12 by fluorescence in situ hybridization. Jpn. J. Genet. 68: 617-621, 1993.166.
Orye, E.; Benoit, Y.; Coppieters, R.; Jeannin, P.; Vercruysse, C.; Delaey, J.; Delbeke, M.-J.: A case of retinoblastoma, associated with histiocytosis-X and mosaicism of a deleted D-group chromosome (13q14-q31). Clin. Genet. 22: 37-39, 1982.167. Orye, E.; Delbeke, M. J.; Vandenabeele, B.: Retinoblastoma and long arm deletion of chromosome 13. Attempts to define the deleted segment. Clin. Genet. 5: 457-464, 1974.168. Orye, E.; Delbeke, M. J.; Vandenabeele, B.: Retinoblastoma and D-chromosome deletions. (Letter) Lancet II: 1376, 1971.169. Otterson, G. A.; Modi, S.; Nguyen, K.; Coxon, A. B.; Kaye, F. J.: Temperature-sensitive RB mutations linked to incomplete penetrance of familial retinoblastoma in 12 families. Am. J. Hum. Genet. 65:1040-1046, 1999.170. Otterson, G. W.; Chen, W.; Coxon, A. B.; Khleif, S. N.; Kaye, F. J.: Incomplete penetrance of familial retinoblastoma linked to germ-line mutations that result in partial loss of RB function. Proc. Nat. Acad. Sci. 94: 12036-12040, 1997.171. Pendergrass, T. W.; Davis, S.: Incidence of retinoblastoma in the United States. Arch. Ophthal. 98: 1204-1210, 1980.172. Pennaneach, V.; Salles-Passador, I.; Munshi, A.; Brickner, H.; Regazzoni, K.; Dick, F.; Dyson, N.; Chen, T.-T.; Wang, J. Y. J.; Fotedar, R.; Fotedar, A.: The large subunit of replication factor C promotes cell survival after DNA damage in an LxCxE motif- and Rb-dependent manner. Molec. Cell 7: 715-727, 2001.173. Riccardi, V. M.; Hittner, H. M.; Francke, U.; Pippin, S.; Holmquist, G. P.; Kretzer, F. L.; Ferrell, R.: Partial triplication and deletion of 13q: study of a family presenting with bilateral retinoblastomas. Clin. Genet. 15: 332-345, 1979.174. Rivera, H.; Turleau, C.; de Grouchy, J.; Junien, C.; Despoisse, S.; Zucker, J.-M.: Retinoblastoma-del(13q14): report of two patients, one with a trisomic sib due to maternal insertion; gene-dosage effect for esterase D. Hum. Genet. 59: 211-214, 1981.175. Sakai, T.; Ohtani, N.; McGee, T. L.; Robbins, P. D.; Dryja, T. P.: Oncogenic germ-line mutations in Sp1 and ATF sites in the human retinoblastoma gene. Nature 353: 83-86, 1991.176. Sakai, T.; Ohtani, N.; McGee, T. L.; Robbins, P. D.; Dryja, T. P.: Oncogenic germ-line mutations in Sp1 and ATF sites in the human retinoblastoma gene. Nature 353: 83-86, 1991.177. Sakai, T.; Toguchida, J.; Ohtani, N.; Yandell, D. W.; Rapaport, J. M.; Dryja, T. P.: Allele-specific hypermethylation of the retinoblastoma tumor-suppressor gene. Am. J. Hum. Genet. 48: 880-888, 1991.178. Schappert-Kimmijser, J.; Hemmes, G. D.; Nijland, R.: The heredity of retinoblastoma. Ophthalmologica 151: 197-213, 1966.179. Scheffer, H.; te Meerman, G. J.; Kruize, Y. C. M.; van den Berg, A. H. M.; Penninga, D. P.; Tan, K. E. W. P.; der Kinderen, D. J.; Buys, C. H. C. M.: Linkage analysis of families with hereditary retinoblastoma: nonpenetrance of mutation, revealed by combined use of markers within and flanking the RB1 gene. Am. J. Hum. Genet. 45: 252-260, 1989.180. Schimke, R. N.; Lowman, J.; Cowan, G.: Retinoblastoma and osteogenics arcoma in sibs. Cancer 34: 2077-2079, 1974.181. Schubert, E. L.; Strong, L. C.; Hansen, M. F.: A splicing mutationin RB1 in low penetrance retinoblastoma. Hum. Genet. 100: 557-563, 1997.182. Shields, C. L.; Honavar, S.; Shields, J. A.; Demirci, H.; Meadows, A. T.: Vitrectomy in eyes with unsuspected retinoblastoma. Ophthalmology 107:2250-2255, 2000.183. Shiio, Y.; Yamamoto, T.; Yamaguchi, N.: Negative regulation of Rb expression by the p53 gene product. Proc. Nat. Acad. Sci. 89:5206-5210, 1992.184. Shroeder, W. T.; Chao, L.-Y.; Dao, D. D.; Strong, L. C.; Pathak, S.; Riccardi, V.; Lewis, W. H.; Saunders, G. F.: Nonrandom loss of maternal chromosome 11 alleles in Wilms tumors. Am. J. Hum. Genet. 40:413-420, 1987.185. Sippel, K. C.; Fraioli, R. E.; Smith, G. D.; Schalkoff, M. E.; Sutherland, J.; Gallie, B. L.; Dryja, T. P.: Frequency of somatic and germ-line mosaicism in retinoblastoma: implications for genetic counseling. Am. J. Hum. Genet. 62: 610-619, 1998.186. Smith, S. M.; Sorsby, A.: Retinoblastoma: some genetic aspects. Ann. Hum. Genet. 23: 50-58, 1958.187. Sparkes, R. S.: The genetics of retinoblastoma. Biochim. Biophys. Acta 780: 95-118, 1985.188. Sparkes, R. S.; Muller, H.; Klisak, I.; Abram, J. A.: Retinoblastoma with 13q; chromosomal deletion associated with maternal paracentric inversion of 13q. Science 203: 1027-1029, 1979.189. Sparkes, R. S.; Murphree, A. L.; Lingua, R. W.; Sparkes, M. C.; Field, L. L.; Funderburk, S. J.; Benedict, W. F.: Gene for hereditary retinoblastoma assigned to human chromosome 13 by linkage to esterase D. Science 219: 971-973, 1983.190.
Sparkes, R. S.; Sparkes, M. C.; Wilson, M. G.; Towner, J. W.; Benedict, W.; Murphree, A. L.; Yunis, J. J.: Regional assignment of genes for human esterase D and retinoblastoma to chromosome band 13q14. Science 208: 1042-1044, 1980.191. Sparkes, R. S.; Sparkes, M. C.; Wilson, M. G.; Towner, J. W.; Benedict, W.; Murphree, A. L.; Yunis, J. J.: Regional assignment of genes for human esterase D and retinoblastoma to chromosome band 13q14. (Abstract) Cytogenet. Cell Genet. 25: 209, 1979.192.
Squire, J.; Gallie, B. L.; Phillips, R. A.: A detailed analysis of chromosomal changes in heritable and non-heritable retinoblastoma. Hum. Genet. 70: 291-301, 1985.193.
Squire, J.; Phillips, R. A.; Boyce, S.; Godbout, R.; Rogers, B.; Gallie, B. L.: Isochromosome 6p, a unique chromosomal abnormality in retinoblastoma: verification by standard staining techniques, new densitometric methods, and somatic cell hybridization. Hum. Genet. 66:46-53, 1984.194. Stallard, H. B.: The conservation treatment of retinoblastoma. Trans. Ophthal. Soc. 82: 473, 1962.195.
Stone, J. C.; Crosby, J. L.; Kozak, C. A.; Schievella, A. R.; Bernards, R.; Nadeau, J. H.: The murine retinoblastoma homolog maps to chromosome 14 near Es-10. Genomics 5: 70-75, 1989.196. Strong, L. C.; Riccardi, V. M.; Ferrell, R. E.; Sparkes, R. S.: Familial retinoblastoma and chromosome 13 deletion transmitted via an insertional translocation. Science 213: 1501-1503, 1981.197. Taylor, A. I.: Dq-, Dr and retinoblastoma. Human genetik 10:209-217, 1970.198. Thomas, D. M.; Carty, S. A.; Piscopo, D. M.; Lee, J.-S.; Wang, W.-F.; Forrester, W. C.; Hinds, P. W.: The retinoblastoma protein acts as a transcriptional coactivator required for osteogenic differentiation. Molec. Cell 8: 303-316, 2001.199. Toguchida, J.; Ishizaki, K.; Sasaki, M. S.; Nakamura, Y.; Ikenaga, M.; Kato, M.; Sugimot, M.; Kotoura, Y.; Yamamuro, T.: Preferential mutation of paternally derived RB gene as the initial event in sporadicosteosarcoma. Nature 338: 156-158, 1989.200. Toguchida, J.; McGee, T. L.; Paterson, J. C.; Eagle, J. R.; Tucker, S.; Yandell, D. W.; Dryja, T. P.: Complete genomic sequence of the human retinoblastoma susceptibility gene. Genomics 17: 535-543, 1993.201. Turleau, C.; de Grouchy, J.; Chavin-Colin, F.; Despoisses, S.; Leblanc, A.: Two cases of del(13q)-retinoblastoma and two cases of partial trisomy due to a familial insertion. Ann. Genet. 26: 158-160, 1983.202. Turleau, C.; de Grouchy, J.; Chavin-Colin, F.; Junien, C.; Seger, J.; Schlienger, P.; Leblanc, A.; Haye, C.: Cytogenetic forms of retinoblastoma: their incidence in a survey of 66 patients. Cancer Genet. Cytogenet. 16:321-334, 1985.203. Verma, R. S.; Ramesh, K. H.; Samonte, R. V.; Conte, R. A.: Mapping the homolog of the human Rb1 gene to chromosome 14 of higher primates. Mammalian Genome 7: 591-592, 1996.204. Vogel, F.: Genetics of retinoblastoma. Modern Trends in Ophthalmology. (pub.) 1968.205. Vogel, F.: Genetics of retinoblastoma. In: Genetic Counseling. Heidelberg University, Science Library. Trans. by Sabine Kurth. New York: Springer Verlag (pub.) 1969.206. Vogel, F.: The genetics of retinoblastoma. Hum. Genet. 52:1-54, 1979.207. Warburg, M.: Retinoblastoma. In: Goldberg, M. F.: Genetic and Metabolic Eye Disease. Boston: Little, Brown and Co. (pub.) 1974. Pp. 447-461.208. Weichselbaum, R. R.; Beckett, M.; Diamond, A.: Some retinoblastomas, osteosarcomas, and soft tissue sarcomas may share a common etiology. Proc. Nat. Acad. Sci. 85: 2106-2109, 1988.209. Weichselbaum, R. R.; Nove, J.; Little, J. B.: Fibroblasts from a D-deletion type retinoblastoma patient are abnormally x-ray sensitive. Nature 266:726-727, 1977.210. Weinberg, R. A.: The retinoblastoma protein and cell cycle control. Cell 81:323-330, 1995.211. Whyte, P.; Buchkovich, K. J.; Horowitz, J. M.; Friend, S. H.; Raybuck, M.; Weinberg, R. A.; Harlow, E.: Association between an oncogene and an anti-oncogene: the adenovirus E1A proteins bind to the retinoblastoma gene product. Nature 334: 124-129, 1988.212. Wiggs, J.; Nordenskjold, M.; Yandell, D.; Rapaport, J.; Grondin, V.; Janson, M.; Werelius, B.; Petersen, R.; Craft, A.; Riedel, K.; Liberfarb, R.; Walton, D.; Wilson, W.; Dryja, T. P.: Prediction of the risk of hereditary retinoblastoma, using DNA polymorphisms within the retinoblastoma gene. New Eng. J. Med. 318: 151-157, 1988.213.
Wilson, M. G.; Ebbin, A. J.; Towner, J. W.; Spencer, W. H.: Chromosomal anomalies in patients with retinoblastoma. Clin. Genet. 12:1-8, 1977.214. Wilson, M. G.; Melnyk, J.; Towner, J. W. J.: Retinoblastoma and deletion D(14) syndrome. J. Med. Genet. 6: 322-327, 1969.215. Wilson, M. G.; Towner, J. W.; Fujimoto, A.: Retinoblastoma and D-chromosome deletions. Am. J. Hum. Genet. 25: 57-61, 1973.216. Windle, J. J.; Albert, D. M.; O'Brien, J. M.; Marcus, D. M.; Disteche, C. M.; Bernards, R.; Mellon, P. L.: Retinoblastoma in transgenic mice. Nature 343: 665-669, 1990.217. Yandell, D. W.; Campbell, T. A.; Dayton, S. H.; Petersen, R.; Walton, D.; Little, J. B.; McConkie-Rosell, A.; Buckley, E.; Dryja, T.: Oncogenic point mutations in the human retinoblastoma gene: their application to genetic counseling. New Eng. J. Med. 321: 1689-1695, 1989.218.
Yokota, J.; Akiyama, T.; Fung, Y.-K. T.; Benedict, W. F.; Namba, Y.; Hanaoka, M.; Wada, M.; Terasaki, T.; Shimosato, Y.; Sugimura, T.; Terada, M.: Altered expression of the retinoblastoma (RB) gene in small-cell carcinoma of the lung. Oncogene 3: 471-475, 1988.219. Zeschnigk, M.; Lohmann, D.; Horsthemke, B.: A PCR test for the detection of hypermethylated alleles at the retinoblastoma locus. J. Med. Genet. 36: 793-794, 1999.220. Zhang, H. S.; Postigo, A. A.; Dean, D. C.: Active transcriptional repression by the Rb-E2F complex mediates G1 arrest triggered by p16(INK4a), TGF-beta, and contact inhibition. Cell 97: 53-61, 1999.221. Zhu, X.; Dunn, J. M.; Phillips, R. A.; Goddard, A. D.; Paton, K. E.; Becker, A.; Gallie, B. L.: Preferential germline mutation of the paternal allele in retinoblastoma. Nature 340: 312-313, 1989.
Baens, M.; Aerssens, J.; Van Z and, K.; Van den Berghe, H.; Marynen, P.: Isolation and regional assignment of human chromosome 12p cDNAs. Genomics 29:44-52, 1995.
Benbrook, D.; Lernhardt, E.; Pfahl, M.: A new retinoic acid receptor identified from a hepatocellular carcinoma. (Letter) Nature 333:669-672, 1988.
Brand, N.; Petkovich, M.; Krust, A.; Chambon, P.; de The, H.; Marchio, A.; Tiollais, P.; Dejean, A.: Identification of a second human retinoic acid receptor. (Letter) Nature 332: 850-853, 1988.
Dejean, A.; Bougueleret, L.; Grzeschik, K.-H.; Tiollais, P.: Hepatitis B virus DNA integration in a sequence homologous to v-erb-A and steroid receptor genes in a hepatocellular carcinoma. Nature 322: 70-72, 1986.
de The, H.; del Mar Vivanco-Ruiz, M.; Tiollais, P.; Stunnenberg, H.; Dejean, A.: Identification of a retinoic acid responsive element in the retinoic acid receptor beta gene. Nature 343: 177-180, 1990.
Aruffo, A.; Seed, B.: Molecular cloning of a CD28 cDNA by a high-efficiency COS cell expression system. Proc. Nat. Acad. Sci. 84: 8573-8577, 1987.
Lafage-Pochitaloff, M.; Costello, R.; Couez, D.; Simonetti, J.; Mannoni, P.; Mawas, C.; Olive, D.: Human CD28 and CTLA-4 Ig superfamily genes are located on chromosome 2 at bands q33-q34. Immunogenetics 31:198-201, 1990.
Lee, K. P.; Taylor, C.; Petryniak, B.; Turka, L. A.; June, C. H.; Thompson, C. B.: The genomic organization of the CD28 gene: implications for the regulation of CD28 mRNA expression and heterogeneity. J. Immun. 145: 344-352, 1990.
Lesslauer, W.; Gmunder, H.; Bohlen, P.: Purification and N-terminal amino acid sequence of the human T90/44 (CD28) antigen. Immunogenetics 27:388-391, 1988.
Okkenhaug, K.; Wu, L.; Garza, K. M.; La Rose, J.; Khoo, W.; Odermatt, B.; Mak, T. W.; Ohashi, P. S.; Rottapel, R.: A point mutation in CD28 distinguishes proliferative signals from survival signals. Nature Immun. 2: 325-332, 2001.
Jourdan-Le Saux, C.; Tomsche, A.; Ujfalusi, A.; Jia, L.; Csiszar, K.: Central nervous system, uterus, heart, and leukocyte expression of the LOXL3 gene, encoding a novel lysyl oxidase-like protein. Genomics 74:211-218, 2001.
Maki, J. M.; Kivirikko, K. I.: Cloning and characterization of a fourth human lysyl oxidase isoenzyme. Biochem. J. 355: 381-387, 2001.
Riewald, M.; Petrovan, R. J.; Donner, A.; Mueller, B. M.; Ruf, W.: Activation of endothelial cell protease activated receptor 1 by the protein C pathway. Science 296: 1880-1882, 2002.
de The, H.; Marchio, A.; Tiollais, P.; Dejean, A.: A novel steroid thyroid hormone receptor-related gene inappropriately expressed in human hepatocellular carcinoma. Nature 330: 667-670, 1987.
Kreczel, W.; Ghyselinck, N.; Samad, T. A.; Dupe, V.; Kastner, P.; Borrelli, E.; Chambon, P.: Impaired locomotion and dopamine signaling in retinoid receptor mutant mice. Science 279: 863-867, 1998.
Lotan, R.; Xu, X.-C.; Lippman, S. M.; Ro, J. Y.; Lee, J. S.; Lee, J. J.; Hong, W. K.: Suppression of retinoic acid receptor-beta in premalignant oral lesions and its up-regulation by isotretinoin. New Eng. J. Med. 332: 1405-1410, 1995.
Mattei, M.-G.; de The, H.; Mattei, J.-F.; Marchio, A.; Tiollais, P.; Dejean, A.: Assignment of the human hap retinoic acid receptor RAR-beta gene to the p24 band of chromosome 3. Hum. Genet. 80: 189-190, 1988.
Nadeau, J. H.; Compton, J. G.; Giguere, V.; Rossant, J.; Varmuza, S.: Close linkage of retinoic acid receptor genes with homeobox- and keratin-encoding genes on paralogous segments of mouse chromosomes 11 and 15. Mammalian Genome 3: 202-208, 1992.
Samad, A.; Kreczel, W.; Chambon, P.; Borrelli, E.: Regulation of dopaminergic pathways by retinoids: activation of the D2 receptor promoter by members of the retinoic acid receptor-retinoid X receptor family. Proc. Nat. Acad. Sci. 94: 14349-14354, 1997.
Sakaguchi, A. Y.; Zabel, B. U.; Grzeschik, K. H.; Law, M. L.; Ellis, R. W.; Skolnick, E. M.; Naylor, S. L.: Regional localization of two human cellular Kirsten ras genes on chromosomes 6 and 12. Molec. Cell. Biol. 4: 989-993, 1984.
Kas, K.; Roijer, E.; Voz, M.; Meyen, E.; Stenman, G.; Van de Ven, W. J. M.: A 2-Mb YAC contig and physical map covering the chromosome 8q12 breakpoint cluster region in pleomorphic adenomas of the salivary glands. Genomics 43: 349-358, 1997.
Kas, K.; Voz, M. L.; Roijer, E.; Astrom, A.-K.; Meyen, E.; Stenman, G.; Van de Ven, W. J. M.: Promoter swapping between the genes for a novel zinc finger protein and beta-catenin in pleiomorphic adenomas with t(3;8)(p21;q12) translocations. Nature Genet. 15: 170-174, 1997.
Szabo, G.; Dallmann, G.; Muller, G.; Patthy, L.; Soller, M.; Varga, L.: A deletion in the myostatin gene causes the compact (Cmpt) hypermuscular mutation in mice. Mammalian Genome 9: 671-672, 1998.
Zimmers, T. A.; Davies, M. V.; Koniaris, L. G.; Haynes, P.; Esquela, A. F.; Tomkinson, K. N.; McPherron, A. C.; Wolfman, N. M.; Lee, S.-J.: Induction of cachexia in mice by systemically administered myostatin. Science 296:1486-1488, 2002.
Kimura, K.; Ito, M.; Amano, M.; Chihara, K.; Fukata, Y.; Nakafuku, M.; Yamamori, B.; Feng, J.; Nakano, T.; Okawa, K.; Iwamatsu, A.; Kaibuchi, K.: Regulation of myosin phosphatase by Rho and Rho-associated kinase (Rho-kinase). Science 273: 245-248, 1996.
Takahashi, N.; Ito, M.; Tanaka, J.; Nakano, T.; Kaibuchi, K.; Odai, H.; Takemura, K.: Localization of the gene coding for myosin phosphatase, target subunit 1 (MYPT1) to human chromosome 12q15-q21. Genomics 44:150-152, 1997.
D'Esposito, M.; Strazzullo, M.; Cuccurese, M.; Spalluto, C.; Rocchi, M.; D'Urso, M.; Ciccodicola, A.: Identification and assignment of the human transient receptor potential channel 6 gene TRPC6 to chromosome 11q21-q22. Cytogenet. Cell Genet. 83: 46-47, 1998.
Hofmann, T.; Obukhov, A. G.; Schaefer, M.; Harteneck, C.; Gudermann, T.; Schultz, G.: Direct activation of human TRPC6 and TRPC3 channels by diacylglycerol. Nature 397: 259-263, 1999.
Tasheva, E. S.; Pettenati, M.; Von Kap-Her, C.; Conrad, G. W.: Assignment of mimecan gene (OGN) to human chromosome band 9q22 by in situ hybridization. Cytogenet. Cell Genet. 88: 326-327, 2000
Pucharcos, C.; Fuentes, J.-J.; Casas, C.; de la Luna, S.; Alcantara, S.; Arbones, M. L.; Soriano, E.; Estivill, X.; Prichard, M.: Alu-splice cloning of human intersect in (ITSN), a putative multivalent binding protein expressed in proliferating and differentiating neurons and overexpressed in Down syndrome. Europ. J. Hum. Genet. 7: 704-712, 1999.
Katz, P.; Whalen, G.; Kehrl, J. H.: Differential expression of a novel protein kinase in human B lymphocytes: preferential localization in the germinal center. J. Biol. Chem. 269: 16802-16809, 1994.
Pombo, C. M.; Kehrl, J. H.; Sanchez, I.; Katz, P.; Avruch, J.; Zon, L. I.; Woodgett, J. R.; Force, T.; Kyriakis, J. M.: Activation of the SAPK pathway by the human STE20 homologue germinal centre kinase. Nature 377:750-754, 1995.
Ren, M.; Zeng, J.; De Lemos-Chiarandini, C.; Rosenfeld, M.; Adesnik, M.; Sabatini, D. D.: In its active form, the GTP-binding protein rab8 interacts with a stress-activated protein kinase. Proc. Nat. Acad. Sci. 93: 5151-5155, 1996.
Sparks, A. B.; Hoffman, N. G.; McConnell, S. J.; Fowlkes, D. M.; Kay, B. K.: Cloning of ligand targets: systematic isolation of SH3 domain-containing proteins. Nature Biotech. 14: 741-744, 1996.
Chang, H. Y.; Nishitoh, H.; Yang, X.; Ichijo, H.; Baltimore, D.: Activation of apoptosis signal-regulating kinase 1 (ASK1) by the adapter protein Daxx. Science 281: 1860-1863, 1998.
Geleziunas, R.; Xu, W.; Takeda, K.; Ichijo, H.; Greene, W. C.: HIV-1 Nef inhibits ASK1-dependent death signalling providing a potential mechanism for protecting the infected host cell. Nature 410: 834-838, 2001.
Ichijo, H.; Nishida, E.; Irie, K.; ten Dijke, P.; Saitoh, M.; Moriguchi, T.; Takagi, M.; Matsumoto, K.; Miyazono, K.; Gotoh, Y.: Induction of apoptosis by ASK1, a mammalian MAPKKK that activates SAPK/JNK and p38 signaling pathways. Science 275: 90-94, 1997.
Nishitoh, H.; Saitoh, M.; Mochida, Y.; Takeda, K.; Nakano, H.; Rothe, M.; Miyazono, K.; Ichijo, H.: ASK1 is essential for JNK/SAPK activation by TRAF2. Molec. Cell 2: 389-395, 1998.
Tomasetto, C.; Regnier, C.; Moog-Lutz, C.; Mattei, M. G.; Chenard, M. P.; Lidereau, R.; Basset, P.; Rio, M. C.: Identification of four novel human genes amplified and overexpressed in breast carcinoma and localized to the q11-q21.3 region of chromosome 17. Genomics 28:367-376, 1995.
Oyake, T.; Itoh, K.; Motohashi, H.; Hayashi, N.; Hoshino, H.; Nishizawa, M.; Yamamoto, M.; Igarashi, K.: Bach proteins belong to a novel family of BTB-basic leucine zipper transcription factors that interact with MafK and regulate transcription through the NF-E2 site. Molec. Cell. Biol. 16: 6083-6095, 1996.
Johnson, L.; Mercer, K.; Greenbaum, D.; Bronson, R. T.; Crowley, D.; Tuveson, D. A.; Jacks, T.: Somatic activation of the K-ras oncogene causes early onset lung cancer in mice. Nature 410: 1111-1116, 2001.
Kozma, S. C.; Bogaard, M. E.; Buser, K.; Saurer, S. M.; Bos, J. L.; Groner, B.; Hynes, N. E.: The human c-Kirsten ras gene is activated by a novel mutation in codon 13 in the breast carcinoma cell line MDA-MB231. Nucleic Acids Res. 15: 5963-5971, 1987.
Laghi, L.; Orbetegli, O.; Bianchi, P.; Zerbi, A.; Di Carlo, V.; Boland, C. R.; Malesci, A.: Common occurrence of multiple K-RAS mutations in pancreatic cancers with associated precursor lesions and in biliary cancers. Oncogene 21: 4301-4306, 2002.
Lee, K.-H.; Lee, J.-S.; Suh, C.; Kim, S.-W.; Kim, S.-B.; Lee, J.-H.; Lee, M.-S.; Park, M.-Y.; Sun, H.-S.; Kim, S.-H.: Clinicopathologic significance of the K-ras gene codon 12 point mutation in stomach cancer: an analysis of 140 cases. Cancer 75: 2794-2801, 1995.
Liu, E.; Hjelle, B.; Morgan, R.; Hecht, F.; Bishop, J. M.: Mutations of the Kirsten-ras proto-oncogene in human preleukaemia. Nature 330:186-188, 1987.
McBride, O. W.; Swand, D. C.; Tronick, S. R.; Gol, R.; Klimanis, D.; Moore, D. E.; Aaronson, S. A.: Regional chromosomal localization of N-ras, K-ras-1, K-ras-2 and myb oncogenes in human cells. Nucleic Acids Res. 11: 8221-8236, 1983.
McCoy, M. S.; Toole, J. J.; Cunningham, J. M.; Chang, E. H.; Lowy, D. R.; Weinberg, R. A.: Characterization of a human colon/lung carcinoma oncogene. Nature 302: 79-81, 1983.
McGrath, J. P.; Capon, D. J.; Smith, D. H.; Chen, E. Y.; Seeburg, P. H.; Goeddel, D. V.; Levinson, A. D.: Structure and organization of the human Ki-ras proto-oncogene and a related processed pseudo gene. Nature 304:501-506, 1983.
Motojima, K.; Urano, T.; Nagata, Y.; Shiku, H.; Tsurifune, T.; Kanematsu, T.: Detection of point mutations in the Kirsten-ras oncogene provides evidence for the multicentricity of pancreatic carcinoma. Ann. Surg. 217: 138-143, 1993
Muller, R.; Slamon, D. J.; Adamson, E. D.; Tremblay, J. M.; Muller, D.; Cline, M. J.; Verma, I. M.: Transcription of c-one genes c-ras(Ki) and c-fms during mouse development. Molec. Cell. Biol. 3: 1062-1069, 1983.
Nakano, H.; Yamamoto, F.; Neville, C.; Evans, D.; Mizuno, T.; Perucho, M.: Isolation of transforming sequences of two human lung carcinomas: structural and functional analysis of the activated c-K-ras oncogenes. Proc. Nat. Acad. Sci. 81: 71-75, 1984.
O'Brien, S. J.; Nash, W. G.; Goodwin, J. L.; Lowry, D. R.; Chang, E. H.: Dispersion of the ras family of transforming genes to four different chromosomes in man. Nature 302: 839-842, 1983.
O'Connell, P.; Leppert, M.; Hoff, M.; Kumlin, E.; Thomas, W.; Cai, G.; Law, M.; White, R.: A linkage map for human chromosome 12. (Abstract) Am. J. Hum. Genet. 37: A169 only, 1985.
Ohashi, H.; Ishikiriyama, S.; Fukushima, Y.: New diagnostic method for Pallister-Killian syndrome: detection of i(12p) in interphase nuclei of buccal mucosa by fluorescence in situ hybridization. Am. J. Med. Genet. 45: 123-128, 1993.
Otori, K.; Oda, Y.; Sugiyama, K.; Hasebe, T.; Mukai, K.; Fujii, T.; Tajiri, H.; Yoshida, S.; Fukushima, S.; Esumi, H.: High frequency of K-ras mutations in human colorectal hyperplastic polyps. Gut 40:660-663, 1997.
Peltomaki, P.; Knuutila, S.; Ritvanen, A.; Kaitila, I.; de la Chapelle, A.: Pallister-Killian syndrome: cytogenetic and molecular studies. Clin. Genet. 31: 399-405, 1987.
Pfeifer, G. P.: A new verdict for an old convict. Nature Genet. 29:3-4, 2001.
Porta, M.; Malats, N.; Jariod, M.; Grimalt, J. O.; Rifa, J.; Carrato, A.; Guarner, L.; Salas, A.; Santiago-Silva, M.; Corominas, J. M.; Andreu, M.; Real, F. X.: Serum concentrations of organochlorine compounds and K-ras mutations in exocrine pancreatic cancer. Lancet 354: 2125-2129, 1999.
Pulciani, S.; Santos, E.; Lauver, A. V.; Long, L. K.; Aaronson, S. A.; Barbacid, M.: Oncogene in solid human tumors. Nature 300:539-542, 1982.
Rodenhuis, S.; van de Wetering, M. L.; Mooi, W. J.; Evers, S. G.; van Zandwijk, N.; Bos, J. L.: Mutational activation of the K-RAS oncogene: a possible pathogenetic factor in adenocarcinoma of the lung. New Eng. J. Med. 317: 929-935, 1987.
Santos, E.; Martin-Zanca, D.; Reddy, E. P.; Pierotti, M. A.; DellaPorta, G.; Barbacid, M.: Malignant activation of a Kras oncogene in lung carcinoma but not in normal tissue of the same patient. Science 223:661-664, 1984.
Schinzel, A.: Tetrasomy 12p (Pallister-Killian syndrome). J. Med. Genet. 28: 122-125, 1991.
Shimizu, K.; Birnbaum, D.; Ruley, M. A.; Fasano, O.; Suard, Y.; Edlund, L.; Taparowsky, E.; Goldfarb, M.; Wigler, M.: Structure of the Ki-ras gene of the human lung carcinoma cell line Calu-1. Nature 304:497-500, 1983.
Sidransky, D.; Tokino, T.; Hamilton, S. R.; Kinzler, K. W.; Levin, B.; Frost, P.; Vogelstein, B.: Identification of RAS oncogene mutations in the stool of patients with curable colorectal tumors. Science 256:102-105, 1992.
Smit, V. T. H. B. M.; Boot, A. J. M.; Smits, A. M. M.; Fleuren, G. J.; Cornelisse, C. J.; Bos, J. L.: KRAS codon 12 mutations occur very frequently in pancreatic adenocarcinomas. Nucleic Acids Res. 16:7773-7782, 1988.
Soukup, S.; Neidich, K.: Prenatal diagnosis of Pallister-Killian syndrome. Am. J. Med. Genet. 35: 526-528, 1990.
Speleman, F.; Leroy, J. G.; Van Roy, N.; De Paepe, A.; Suijkerbuijk, R.; Brunner, H.; Looijenga, L.; Verschraegen-Spae, M.-R.; Orye, E.: Pallister-Killian syndrome: characterization of the isochromosome 12p by fluorescent in situ hybridization. Am. J. Med. Genet. 41:381-387, 1991.
Takeda, S.; Ichii, S.; Nakamura, Y.: Detection of K-ras mutationin sputum by mutant-allele-specific amplification (MASA). Hum. Mutat. 2:112-117, 1993.
Weinberg, R. A.: Fewer and fewer oncogenes. Cell 30: 3-4, 1982.
Wenger, S. L.; Boone, L. Y.; Steele, M. W.: Mosaicism in Pallisteri (12p) syndrome. Am. J. Med. Genet. 35: 523-525, 1990.
Wingo, P. A.; Ries, L. A. G.; Giovino, G. A.; Miller, D. S.; Rosenberg, H. M.; Shopland, D. R.; Thun, M. J.; Edwards, B. K.: Annual report to the nation on the status of cancer, 1973-1996, with a special section on lung cancer and tobacco smoking. J. Nat. Cancer Inst. 91: 675-690, 1999.
Yanez, L.; Groffen, J.; Valenzuela, D. M.: c-K-ras mutations in human carcinomas occur preferentially in codon 12. Oncogene 1:315-318, 1987.
Zakowski, M. F.; Wright, Y.; Ricci, A., Jr.: Pericardial agenesis and focal aplasia cutis in tetrasomy 12p (Pallister-Killian syndrome). Am. J. Med. Genet. 42: 323-325, 1992.
Zhang, J.; Marynen, P.; Devriendt, K.; Fryns, J.-P.; Van den Berghe, H.; Cassiman, J.-J.: Molecular analysis of the isochromosome 12p in the Pallister-Killian syndrome: construction of a mouse-human hybrid cell line containing an i(12p) as the sole human chromosome. Hum. Genet. 83: 359-363, 1989.
Zhang, Z.; Wang, Y.; Vikis, H. G.; Johnson, L.; Liu, G.; Li, J.; Anderson, M. W.; Sills, R. C.; Hong, H. L.; Devereux, T. R.; Jacks, T.; Guan, K.-L.; You, M.: Wildtype Kras2 can inhibit lung carcinogenesis in mice. Nature Genet. 29: 25-33, 2001.
Lu, T. T.; Makishima, M.; Repa, J. J.; Schoonjans, K.; Kerr, T. A.; Auwerx, J.; Mangelsdorf, D. J.: Molecular basis for feedback regulation of bile acid synthesis by nuclear receptors. Molec. Cell 6:507-515, 2000.
Repa, J. J.; Turley, S. D.; Lobaccaro, J.-M. A.; Medina, J.; Li, L.; Lustig, K.; Shan, B.; Heyman, R. A.; Dletschy, J. M.; Mangelsdorf, D. J.: Regulation of absorption and ABC1-mediated efflux of cholesterol by RXR heterodimers. Science 289: 1524-1529, 2000.
Hymowitz, S. G.; Christinger, H. W.; Fuh, G.; Ultsch, M.; O'Connell, M.; Kelley, R. F.; Ashkenazi, A.; de Vos, A. M.: Triggering cell death: the crystal structure of Apo2L/TRAIL in a complex with death receptor 5. Molec. Cell 4: 563-571, 1999.
Anguita, J.; Chalfant, M. L.; Civan, M. M.; Coca-Prados, M.: Molecular cloning of the human volume-sensitive chloride conductance regulatory protein, pI(Cln), from ocular ciliary epithelium. Biochem. Biophys. Res. Commun. 208: 89-95, 1995.
Buyse, G.; De Greef, C.; Raeymaekers, L.; Droogmans, G.; Nilius, B.; Eggermont, J.: The ubiquitously expressed pI(Cln) protein forms homomeric complexes in vitro. Biochem. Biophys. Res. Commun. 218:822-827, 1996.
Nagl, U. O.; Erdel, M.; Schmarda, A.; Seri, M.; Pinggera, G. M.; Gschwentner, M.; Duba, C.; Galietta, L. J. V.; Deetjen, P.; Utermann, G.; Paulmichl, M.: Chromosomal localization of the genes (CLNS1A and CLNS1B) coding for the swelling-dependent chloride channel I (Cln). Genomics 38:438-441, 1996.
Schwartz, R. S.; Rybicki, A. C.; Nagel, R. L.: Molecular cloning and expression of a chloride channel-associated protein pI(Cln) in human young red blood cells: association with actin. Biochem. J. 327:609-616, 1997.
Biunno, I.; Appierto, V.; Cattaneo, M.; Leone, B. E.; Balzano, G.; Socci, C.; Saccone, S.; Letizia, A.; Valle, G. D.; Sgaramella, V.: Isolation of a pancreas-specific gene located on human chromosome 14q31: expression analysis in human pancreatic ductal carcinomas. Genomics 46:284-286, 1997
Donoviel, D. B.; Bernstein, A.: SEL-1L maps to human chromosome 14, near the insulin-dependent diabetes mellitus locus 11. Genomics 56:232-233, 1999.
Grant, B.; Greenwald, I.: The Caenorhabditis. elegans sel-1 gene, a negative regulator of lin-12 and glp-1, encodes a predicted extracellular protein. Genetics 143: 237-247, 1996.
Grant, B.; Greenwald, I.: Structure, function and expression of SEL-1, a negative regulator of LIN-12 and GLP-1 in C. elegans. Development 124:637-644, 1997.
O'Carroll, D.; Scherthan, H.; Peters, A. H. F. M.; Opravil, S.; Haynes, A. R.; Laible, G.; Rea, S.; Schmid, M.; Lebersorger, A.; Jerratsch, M.; Sattler, L.; Mattei, M. G.; Denny, P.; Brown, S. D. M.; Schweizer, D.; Jenuwein, T.: Isolation and characterization of Suv39h2, a second histone H3 methyltransferase gene that displays testis-specific expression. Molec. Cell. Biol. 20: 9423-9433, 2000.
Drewes, G.; Ebneth, A.; Preuss, U.; Mandelkow, E. M.; Mandelkow, E.: MARK, a novel family of protein kinases that phosphorylate microtubule-associated proteins and trigger microtubule disruption. Cell 89: 297-308, 1997.
Nagase, T.; Kikuno, R.; Ishikawa, K.; Hirosawa, M.; Ohara, O.: Prediction of the coding sequences of unidentified human genes. XVII. The complete sequences of 100 new cDNA clones from brain which code for large proteins in vitro. DNA Res. 7: 143-150, 2000.
Yoshikawa, T.; Sanders, A. R.; Esterling, L. E.; Detera-Wadleigh, S. D.: Multiple transcriptional variants and RNA editing in C18orf1, a novel gene with LDLRA and transmembrane domains on 18p11.2. Genomics 47:246-257, 1998.
Yoshikawa, T.; Sanders, A. R.; Esterling, L. E.; Overharser, J.; Garnes, J. A.; Lennon, G.; Grewal, R.; Detera-Wadleigh, S. D.: Isolation of chromosome 18-specific brain transcripts as positional candidates for bipolar disorder. Am. J. Med. Genet. (Neuropsych. Genet.) 74:140-149, 1997.
Anneren, C.; Reedquist, K. A.; Bos, J. L.; Welsh, M.: GTK, a Src-related tyrosine kinase, induces nerve growth factor-independent neurite outgrowth in PC12 cells through activation of the Rapl pathway: relationship to Shb tyrosine phosphorylation and elevated levels of focal adhesion kinase. J. Biol. Chem. 275: 29153-29161, 2000.
Cance, W. G.; Craven, R. J.; Bergman, M.; Xu, L.; Alitalo, K.; Liu, E. T.: Rak, a novel nuclear tyrosine kinase expressed in epithelial cells. Cell Growth Differ. 5: 1347-1355, 1994
Cance, W. G.; Craven, R. J.; Weiner, T. M.; Liu, E. T.: Novel protein kinases expressed in human breast cancer. Int. J. Cancer 54:571-577, 1993.
Lee, J.; Wang, Z.; Luoh, S.-M.; Wood, W. I.; Scadden, D. T.: Cloning of FRK, a novel human intracellular SRC-like tyrosine kinase-encoding gene. Gene 138: 247-251, 1994.
Scott, A. F.: Personal Communication. Baltimore, Md. Dec. 19, 2001.
Gao, J.; Yu, L.; Zhang, P.; Jiang, J.; Chen, J.; Peng, J.; Wei, Y.; Zhao, S.: Cloning and characterization of human and mouse mitochondrial elongation factor G, GFM and Gfm, and mapping of GFM to human chromosome 3q25.1-q26.2. Genomics 74: 109-114, 2001.
Miura, K.; Jacques, K. M.; Stauffer, S.; Kubosaki, A.; Zhu, K.; Hirsch, D. S.; Resau, J.; Zheng, Y.; Randazzo, P. A.: ARAP1: a point of convergence for Arf and Rho signaling. Molec. Cell 9: 109-119, 2002.
Koontz, J. I.; Soreng, A. L.; Nucci, M.; Kuo, F. C.; Pauwels, P.; van den Berghe, H.; Cin, P. D.; Fletcher, J. A.; Sklar, J.: Frequent fusion of the JAZF1 and JJAZ1 genes in endometrial stromal tumors. Proc. Nat. Acad. Sci. 98: 6348-6353, 2001.
Fitzgerald, K. A.; Palsson-McDermott, E. M.; Bowie, A. G.; Jefferies, C. A.; Mansell, A. S.; Brady, G.; Brint, E.; Dunne, A.; Gray, P.; Harte, M. T.; McMurray, D.; Smith, D. E.; Sims, J. E.; Bird, T. A.; O'Neill, L. A. J.: Mal (MyD88-adapter-like) is required for Toll-like receptor-4 signal transduction. Nature 413: 78-83, 2001.
Horng, T.; Barton, G. M.; Medzhitov, R.: TIRAP: an adapter molecule in the Toll signaling pathway. Nature Immun. 2: 835-841, 2001.
Mirzayans, F.; Pearce, W. G.; MacDonald, I. M.; Walter, M. A.: Mutation of the PAX6 gene in patients with autosomal dominant keratitis. Am. J. Hum. Genet. 57: 539-548, 1995.
Liang, J. C.; Chang, K. S.; Schroeder, W.; Siciliano, M.; Trujillo, J.; Stass, S.: The human myeloperoxidase gene locates on chromosome 17q22-24 and is translocated in acute promyelocytic leukemia. (Abstract) Am. J. Hum. Genet. 41: A226, 1987.
Liang, J. C.; Chang, K. S.; Schroeder, W. T.; Freireich, E. J.; Stass, S. A.; Trujillo, J. M.: The myeloperoxidase gene is translocated from chromosome 17 to 15 in a patient with acute promyelocytic leukemia. Cancer Genet. Cytogenet. 30: 103-107, 1988.
Miki, T.; Weil, S. C.; Rosner, G. L.; Reid, M. S.; Kidd, K. K.: An MPO cDNA clone identifies an RFLP with PstI. Nucleic Acids Res. 16:1649, 1988.
Morishita, K.; Kubota, N.; Asano, S.; Kaziro, Y.; Nagata, S.: Molecular cloning and characterization of cDNA for human myeloperoxidase. J. Biol. Chem. 262: 3844-3851, 1987.
Murao, S.-I.; Stevens, F. J.; Ito, A.; Huberman, E.: Myeloperoxidase: a myeloid cell nuclear antigen with DNA-binding properties. Proc. Nat. Acad. Sci. 85: 1232-1236, 1988.
Nauseef, W.; Cogley, M.; McCormick, S.: Effect of the R569W missense mutation on the biosynthesis of myeloperoxidase. J. Biol. Chem. 271:9546-9549, 1996.
Nauseef, W. M.; Olsson, I.; Arnljots, K.: Biosynthesis and processing of myeloperoxidase—a marker for myeloid cell differentiation. Europ. J. Haemat. 40: 97-110, 1988.
Reynolds, W. F.; Hiltunen, M.; Pirskanen, M.; Mannermaa, A.; Helisalmi, S.; Lehtovirta, M.; Alafuzoff, I.; Soininen, H.: MPO and APOE epsilon-4 polymorphisms interact to increase risk for AD in Finnish males. Neurology 55:1284-1290, 2000.
Robinson, T. J.; Morris, D. J.; Ledbetter, D. H.: Chromosomal assignment and regional localization of myeloperoxidase in the mouse. Cytogenet. Cell Genet. 53: 83-86, 1990.
Romano, M.; Dri, P.; Dadalt, L.; Patriarca, P.; Baralle, F. E.: Biochemical and molecular characterization of hereditary myeloproliferative deficiency. Blood 90: 4126-4134, 1997.
van Tuinen, P.; Johnson, K. R.; Ledbetter, S. A.; Nussbaum, R. L.; Rovera, G.; Ledbetter, D. H.: Localization of myeloperoxidase to the long arm of human chromosome 17: relationship to the 15;17 translocation of acute promyelocytic leukemia. Oncogene 1: 319-322, 1987.
Weil, S. C.; Rosner, G. L.; Reid, M. S.; Chisholm, R. L.; Farber, N. M.; Spitznagel, J. K.; Swanson, M. S.: cDNA cloning of human myeloperoxidase: decrease in myeloperoxidase mRNA upon induction of HL-60 cells. Proc. Nat. Acad. Sci. 84: 2057-2061, 1987.
Weil, S. C.; Rosner, G. L.; Reid, M. S.; Chisholm, R. L.; Lemons, R. S.; Swanson, M. S.; Carrino, J. J.; Diaz, M. O.; Le Beau, M. M.: Translocation and rearrangement of myeloperoxidase gene in acute promyelocytic leukemia. Science 240: 790-792, 1988.
Yamada, M.; Hur, S.-J.; Hashinaka, K.; Tsuneoka, K.; Saeki, T.; Nishio, C.; Sakiyama, F.; Tsunasawa, S.: Isolation and characterization of a cDNA coding for human myeloperoxidase. Arch. Biochem. Biophys. 255:147-155, 1987
Zaki, S. R.; Austin, G. E.; Chan, W. C.; Conaty, A. L.; Trusler, S.; Trappier, S.; Lindsey, R. B.; Swan, D. C.: Chromosomal localization of the human myeloperoxidase gene by in situ hybridization using oligonucleotide probes. Genes Chromosomes Cancer 2: 266-270, 1990.
Nishida, K.; Yoshida, Y.; Itoh, M.; Fukada, T.; Ohtani, T.; Shirogane, T.; Atsumi, T.; Takahashi-Tezuka, M.; Ishihara, K.; Hibi, M.; Hirano, T.: Gab-family adapter proteins act downstream of cytokine and growth factor receptors and T- and B-cell antigen receptors. Blood 93:1809-1816, 1999.
Zhao, C.; Yu, D.-H.; Shen, R.; Feng, G.-S.: Gab2, a new pleckstrin homology domain-containing adapter protein, acts to uncouple signaling from ERK kinase to Elk-1. J. Biol. Chem. 274: 19649-19654, 1999.
Nosaka, K.; Onozuka, M.; Kakazu, N.; Hibi, S.; Nishimura, H.; Nishino, H.; Abe, T.: Isolation and characterization of a human thiamine pyrophosphokinase cDNA. Biochem. Biophys. Acta 1517: 293-297, 2001.
Nosaka, K.; Onozuka, M.; Nishino, H.; Nishimura, H.; Kawasaki, Y.; Ueyama, H.: Molecular cloning and expression of a mouse thiamin pyrophosphokinase cDNA. J. Biol. Chem. 274: 34129-34133, 1999
Zhao, R.; Gao, F.; Goldman, I. D.: Molecular cloning of human thiamin pyrophosphokinase. Biochim. Biophys. Acta 1517: 320-322, 2001.
Reboul, J.; Gardiner, K.; Monneron, D.; Uze, G.; Lutfalla, G.: Comparative genomic analysis of the interferon/interleukin-10 receptor gene cluster. Genome Res. 9: 242-250, 1999.
Koyama, K.; Sudo, K.; Nakamura, Y.: Isolation of 115 human chromosome 8-specific expressed-sequence tags by exon amplification. Genomics 26:245-253, 1995.
Pata, I.; Tensing, K.; Metspalu, A.: A human cDNA encoding the homologue of NADH: ubiquinone oxidoreductase subunit B13. Biochim. Biophys. Acta 1350: 115-118, 1997.
Russell, M. W.; du Manoir, S.; Collins, F. S.; Brody, L. C.: Cloning of the human NADH: ubiquinone oxidoreductase subunit B13: localization to chromosome 7q32 and identification of a pseudo gene on 11p15. Mammalian Genome 8: 60-61, 1997.
Lee, J. W.; Choi, H.-S.; Gyuris, J.; Brent, R.; Moore, D. D.: Two classes of proteins dependent on either the presence or absence of thyroid hormone for interaction with the thyroid hormone receptor. Molec. Endocr. 9: 243-254, 1995
Venturini, L.; You, J.; Stadler, M.; Galien, R.; Lallemand, V.; Koken, M. H. M.; Mattei, M. G.; Ganser, A.; Chambon, P.; Losson, R.; de The, H.: TIF1-gamma, a novel member of the transcriptional intermediary factor 1 family. Oncogene 18: 1209-1217, 1999.
Pizutti, A.; Novelli, G.; Ratti, A.; Amati, F.; Mari, A.; Calabrese, G.; Nicolis, S.; Silani, V.; Marino, B.; Scarlato, G.; Ottolenghi, S.; Dallapiccola, B.: UFD1L, a developmentally expressed ubiquitination gene, is deleted in CATCH 22 syndrome. Hum. Molec. Genet. 6: 259-265, 1997.
Yamagishi, H.; Garg, V.; Matsuoka, R.; Thomas, T.; Srivastava, D.: A molecular pathway revealing a genetic basis for human cardiac and craniofacial defects. Science 283: 1158-1161, 1999.
Teumer, J.; Tseng, H.; Green, H.: The human basonuclin gene. Gene 188:1-7, 1997.
Tseng, H.; Green, H.: Basonuclin: a keratinocyte protein with multiple paired zinc fingers. Proc. Nat. Acad. Sci. 89: 10311-10315, 1992.
Tseng, H.; Green, H.: Association of basonuclin with ability of keratinocytes to multiply and with absence of terminal differentiation. J. Cell Biol. 126: 495-506, 1994.
Okamura, H.; Miyake, S.; Sumi, Y.; Yamaguchi, S.; Yasui, A.; Muijtjens, M.; Hoeijmakers, J. H. J.; van der Horst, G. T. J.: Photic induction of mPer1 and mPer2 in Cry-deficient mice lacking a biological clock. Science 286:2531-2534, 1999.
Reick, M.; Garcia, J. A.; Dudley, C.; McKnight, S. L.: NPAS2: an analog of clock operative in the mammalian forebrain. Science 293:506-509, 2001.
Fardaei, M.; Rogers, M. T.; Thorpe, H. M.; Larkin, K.; Hamshere, M. G.; Harper, P. S.; Brook, J. D.: Three proteins, MBNL, MBLL and MBXL, co-localize in vivo with nuclear foci of expanded-repeat transcripts in DM1 and DM2 cells. Hum. Molec. Genet. 11: 805-814, 2002.
Miller, J. W.; Urbinati, C. R.; Teng-umnuay, P.; Stenberg, M. G.; Byrne, B. J.; Thornton, C. A.; Swanson, M. S.: Recruitment of human muscle blind proteins to (CUG)n expansions associated with myotonic dystrophy. EMBO J. 19: 4439-4448, 2000.
Imataka, H.; Olsen, H. S.; Sonenberg. N.: A new translational regulator with homology to eukaryotic translation initiation factor 4G. EMBO J. 16: 817-825, 1997.
Levy-Strumpf, N.; Deiss, L. P.; Berissi, H.; Kimchi, A.: DAP-5, a novel homolog of eukaryotic translation initiation factor 4G isolated as a putative modulator of gamma interferon-induced programmed cell death. Molec. Cell. Biol. 17: 1615-1625, 1997.
Shaughnessy, J. D., Jr.; Jenkins, N. A.; Copeland, N. G.: cDNA cloning, expression analysis, and chromosomal localization of a gene with high homology to wheat eIF-(iso)₄F and mammalian eIF-4G. Genomics 39:192-197, 1997.
Yamanaka, S.; Poksay, K. S.; Arnold, K. S.; Innerarity, T. L.: A novel translational repressor mRNA is edited extensively in livers containing tumors caused by the trans gene expression of the apoB mRNA-editing enzyme. Genes Dev. 11: 321-333, 1997.
Westendorf, J. M.; Rao, P. N.; Gerace, L.: Cloning of cDNAs for M-phase phosphoproteins recognized by the MPM2 monoclonal antibody and determination of the phosphorylated epitope. Proc. Nat. Acad. Sci. 91: 714-718, 1994.
Wang, X. S.; Diener, K.; Jannuzzi, D.; Trollinger, D.; Tan, T.-H.; Lichenstein, H.; Zukowski, M.; Yao, Z.: Molecular cloning and characterization of a novel protein kinase with a catalytic domain homologous to mitogen-activated protein kinase kinase kinase. J. Biol. Chem. 271: 31607-31611, 1996.
Bromme, D.; Rossi, A. B.; Smeekens, S. P.; Anderson, D. C.; Payan, D. G.: Human bleomycin hydrolase: molecular cloning, sequencing, functional expression, and enzymatic characterization. Biochemistry 35:6706-6714, 1996.
Cloos, J.; Nieuwenhuis, E. J. C.; Boomsma, D. I.; Kuik, D. J.; van der Sterre, M. L. T.; Arwert, F.; Snow, G. B.; Braakhuis, B. J. M.: Inherited susceptibility to bleomycin-induced chromatid breaks in cultured peripheral blood lymphocytes. J. Nat. Cancer Inst. 91:1125-1130, 1999.
Farrer, L. A.; Abraham, C. R.; Haines, J. L.; Rogaeva, E. A.; Song, Y.; McGraw, W. T.; Brindle, N.; Premkumar, S.; Scott, W. K.; Yamaoka, L. H.; Saunders, A. M.; Roses, A. D.; Auerbach, S. A.; Sorbi, S.; Duara, R.; Pericak-Vance, M. A.; St. George-Hyslop, P. H.: Association between bleomycin hydrolase and Alzheimer's disease in Caucasians. Ann. Neurol. 44: 808-811, 1998.
Ferrando, A. A.; Pendas, A. M.; Llano, E.; Velasco, G.; Lidereau, R.; Lopez-Otin, C.: Gene characterization, promoter analysis, and chromosomal localization of human bleomycin hydrolase. J. Biol. Chem. 272:33298-33304, 1997.
Ferrando, A. A.; Velasco, G.; Campo, E.; Lopez-Otin, C.: Cloning and expression analysis of human bleomycin hydrolase, a cysteine proteinase involved in chemotherapy resistance. Cancer Res. 56: 1746-1750, 1996
Haston, C. K.; Amos, C. I.; King, T. M.; Travis, E. L.: Inheritance of susceptibility to bleomycin-induced pulmonary fibrosis in the mouse. Cancer Res. 56: 2596-2601, 1996.
Hsu, T. C.; Johnston, D. A.; Cherry, L. M.; Ramkissoon, D.; Schantz, S. P.; Jessup, J. M.; Winn, R. J.; Shirley, L.; Furlong, C.: Sensitivity to genotoxic effects of bleomycin in humans: possible relationship to environmental carcinogenesis. Int. J. Cancer 43: 403-409, 1989.
Lazo, J. S.; Humphreys, C. J.: Lack of metabolism as the biochemical basis of bleomycin-induced pulmonary toxicity. Proc. Nat. Acad. Sci. 80:3064-3068, 1983.
Montoya, S. E.; Aston, C. E.; DeKosky, S. T.; Kamboh, M. I.; Lazo, J. S.; Ferrell, R. E.: Bleomycin hydrolase is associated with risk of sporadic Alzheimer's disease. (Letter) Nature Genet. 18: 211-212, 1998. Note: Erratum: Nature Genet. 19: 404 only, 1998.
Montoya, S. E.; Ferrell, R. E.; Lazo, J. S.: Genomic structure and genetic mapping of the human neutral cysteine protease bleomycin hydrolase. Cancer Res. 57: 4191-4195, 1997.
Zheng, W.; Johnston, S. A.; Joshua-Tor, L.: The unusual active site of Gal6/bleomycin hydrolase can act as a carboxypeptidase, aminopeptidase, and peptide ligase. Cell 93: 103-109, 1998.
Xu, X.-N.; Screaton, G. R.; Gotch, F. M.; Dong, T.; Tan, R.; Almond, N.; Walker, B.; Stebbings, R.; Kent, K.; Nagata, S.; Stott, J. E.; McMichael, A. J.: Evasion of cytotoxic T lymphocyte (CTL) responses by Nef-dependent induction of Fas ligand (CD95L) expression on simian immunodeficiency virus-infected cells. J. Exp. Med. 186: 7-16, 1997.
Wang, B.; Kishihara, K.; Zhang, D.; Hara, H.; Nomoto, K.: Molecular cloning and characterization of a novel human receptor protein tyrosine phosphatase gene, hPTP-J: down-regulation of gene expression by PMA and calcium ionophore in Jurkat T lymphoma cells. Biochem. Biophys. Res. Commun. 231: 77-81, 1997.
Wang, H; Lian, Z; Lerch, M. M.; Chen, Z; Xie, W; Ullrich, A.: Characterization of PCP-2, a novel receptor protein tyrosine phosphatase of the MAM domain family. Oncogene 12: 2555-2562, 1996.
Khoja, H.; Wang, G.; Ng, C.-T. L.; Tucker, J.; Brown, T.; Shyamala, V.: Cloning of CCRL1, an orphan seven transmembrane receptor related to chemokine receptors, expressed abundantly in the heart. Gene 246:229-238, 2000.
Schweickart, V. L.; Epp, A.; Raport, C. J.; Gray, P. W.: CCR11 is a functional receptor for the monocyte chemoattractant protein family of chemokines. J. Biol. Chem. 275: 9550-9556, 2000. Note: Erratum: J. Biol. Chem. 276: 856 only, 2001.
Ungar, D.; Oka, T.; Brittle, E. E.; Vasile, E.; Lupashin, V. V.; Chatterton, J. E.; Heuser, J. E.; Krieger, M.; Waters, M. G.: Characterization of a mammalian Golgi-localized protein complex, COG, that is required for normal Golgi morphology and function. J. Cell Biol. 157: 405-415, 2002.
Dabovic, B.; Chen, Y.; Colarossi, C.; Obata, H.; Zambuto, L.; Perle, M. A.; Rifkin, D. B.: Bone abnormalities in latent TGF-beta binding protein (Ltbp)-3-null mice indicate a role for Ltbp-3 in modulating TGF-beta bioavailability. J. Cell Biol. 156: 227-232, 2002.
Li, X.; Yin, W.; Perez-Jurado, L.; Bonadio, J.; Francke, U.: Mapping of human and murine genes for latent TGF-beta binding protein-2 (LTBP2). Mammalian Genome 6: 42-45, 1995.
Sawicki, M.; Arnold, E.; Ebrahimi, S.; Duell, T.; Jin, S.; Wood, T.; Chakrabarti, R.; Peters, J.; Wan, Y.; Samara, G.; Weier, H.-U. G.; Udar, N.; Passaro, E., Jr.; Srivatsan, E. S.: A transcript map encompassing the multiple endocrine neoplasia type-1 (MEN1) locus on chromosome 11q13. Genomics 42: 405-412, 1997.
Yin, W.; Smiley, E.; Germiller, J.; Mechan, R. P.; Florer, J. B.; Wenstrup, R. J.; Bonadio, J.: Isolation of a novel latent transforming growth factor-beta binding protein gene (LTBP-3). J. Biol. Chem. 270:10147-10160, 1995.
Bussemakers, M. J. G.; van Bokhoven, A.; Voller, M.; Smit, F. P.; Schalken, J. A.: The genes for the calcium-dependent cell adhesion molecules P- and E-cadherin are tandemly arranged in the human genome. Biochem. Biophys. Res. Commun. 203: 1291-1294, 1994.
Carmeliet, P.; Lampugnani, M.-G.; Moons, L.; Breviario, F.; Compernolle, V.; Bono, F.; Balconi, G.; Spagnuolo, R.; Oosthuyse, B.; Dewerchin, M.; Zanetti, A.; Angellilo, A.; and 11 others: Targeted deficiency of cytosolic truncation of the VE-cadherin gene in mice impairs VEGF-mediated endothelial survival and angiogenesis. Cell 98: 147-157, 1999.
Huber, P.; Dalmon, J.; Engiles, J.; Breviario, F.; Gory, S.; Siracusa, L. D.; Buchberg, A. M.; Dejana, E.: Genomic structure and chromosomal mapping of the mouse VE-cadherin gene (Cdh5). Genomics 32: 21-28, 1996.
Salomon, D.; Ayalon, O.; Patel-King, R.; Hynes, R. O.; Geiger, B.: Extra junctional distribution of N-cadherin in cultured human endothelial cells. J. Cell Sci. 102: 7-17, 1992.
Castagnola, P.; Gennari, M.; Morello, R.; Tonachini, L.; Marin, O.; Gaggero, A.; Cancedda, R.: Cartilage associated protein (CASP) is a novel developmentally regulated chick embryo protein. J. Cell Sci. 110: 1351-1359, 1997.
Morello, R.; Tonachini, L.; Monticone, M.; Viggiano, L.; Rocchi, M.; Cancedda, R.; Castagnola, P.: cDNA cloning, characterization and chromosome mapping of Crtap encoding the mouse cartilage associated protein. Matrix Biol. 18: 319-324, 1999.
Tonachini, L.; Morello, R.; Monticone, M.; Skaug, J.; Scherer, S. W.; Cancedda, R.; Castagnola, P.: cDNA cloning, characterization and chromosome mapping of the gene encoding human cartilage associated protein (CRTAP). Cytogenet. Cell Genet. 87: 191-194, 1999.
Shibanuma, M.; Mashimo, J.; Mita, A.; Kuroki, T.; Nose, K.: Cloning from a mouse osteoblastic cell line of a set of transforming-growth-factor-beta-1-regulated genes, one of which seems to encode a follistatin-related polypeptide. Europ. J. Biochem. 217: 13-19, 1993.
Tanaka, M.; Ozaki, S.; Osakada, F.; Mori, K.; Okubo, M.; Nakao, K.: Cloning of follistatin-related protein as a novel autoantigen in systemic rheumatic diseases. Int. Immun. 10: 1305-1314, 1998.
Zwijsen, A.; Blockx, H.; van Arnhem, W.; Willems, J.; Fransen, L.; Devos, K.; Raymackers, J.; van de Voorde, A.; Slegers, H.: Characterization of a rat C6 glioma-secreted follistatin-related protein (FRP): cloning and sequence of the human homologue. Europ. J. Biochem. 225: 937-946, 1994.
Bartles, J. R.; Wierda, A.; Zheng, L.: Identification and characterization of espin, an actin-binding protein localized to the F-actin-rich junctional plaques of Sertoli cell ectoplasmic specializations. J. Cell Sci. 109:1229-1239, 1996.
Bartles, J. R.; Zheng, L.; Li, A.; Wierda, A.; Chen, B.: Smallespin: a third actin-bundling protein and potential forked protein ortholog in brush border microvilli. J. Cell Biol. 143: 107-119, 1998.
Chen, B.; Li, A.; Wang, D.; Wang, M.; Zheng, L.; Bartles, J. R.: Espin contains an additional actin-binding site in its N terminus and is a major actin-bundling protein of the Sertoli cell-spermatid ectoplasmic specialization junctional plaque. Molec. Biol. Cell 10:4327-4339, 1999.
Zheng, L.; Sekerkova, G.; Vranich, K.; Tilney, L. G.; Mugnaini, E.; Bartles, J. R.: The deaf jerker mouse has a mutation in the gene encoding the espin actin-bundling proteins of hair cell stereocilia and lacks espins. Cell 102: 377-385, 2000.
Parker, N. J.; Begley, C. G.; Smith, P. J.; Fox, R. M.: Molecular cloning of a novel human gene (D11S4896E) at chromosomal region 11p15.5. Genomics 37:253-256, 1996.
Sabbioni, S.; Veronese, A.; Trubia, M.; Taramelli, R.; Barbanti-Brodano, G.; Croce, C. M.; Negrini, M.: Exon structure and promoter identification of STIM1 (alias GOK), a human gene causing growth arrest of the human tumor cell lines G401 and RD. Cytogenet. Cell. Genet. 86: 214-218, 1999.
Cao, H.; Hegele, R. A.: Identification of single-nucleotide polymorphisms in the human LPIN1 gene. J. Hum. Genet. 47: 370-372, 2002.
Peterfy, M.; Phan, J.; Xu, P.; Reue, K.: Lipodystrophy in the fld mouse results from mutation of a new gene encoding a nuclear protein, lipin. Nature Genet. 27: 121-124, 2001.
Reue, K.; Xu, P.; Wang, X.-P.; Slavin, B. G.: Adipose tissue deficiency, glucose intolerance, and increased atherosclerosis result from mutationin the mouse fatty liver dystrophy (fld) gene. J. Lipid Res. 41:1067-1076, 2000.
Bodine, S. C.; Latres, E.; Baumhueter, S.; Lai, V. K.-M.; Nunez, L.; Clarke, B. A.; Poueymirou, W. T.; Panaro, F. J.; Na, E.; Dharmarajan, K.; Pan, Z.-Q.; Valenzuela, D. M.; DeChiara, T. M.; Stitt, T. N.; Yancopoulos, G. D.; Glass, D. J.: Identification of ubiquitin ligases required for skeletal muscle atrophy. Science 294: 1704-1708, 2001.
Centner, T.; Yano, J.; Kimura, E.; McElhinny, A. S.; Pelin, K.; Witt, C. C.; Bang, M.-L.; Trombitas, K.; Granzier, H.; Gregorio, C. C.; Sorimachi, H.; Labeit, S.: Identification of muscle specific ring finger proteins as potential regulators of the titin kinase domain. J. Molec. Biol. 306: 717-726, 2001.
Kirikoshi, H.; Koike, J.; Sagara, N.; Saitoh, T.; Tokuhara, M.; Tanaka, K.; Sekihara, H.; Hirai, M.; Katoh, M.: Molecular cloning and genomic structure of human Frizzled-3 at chromosome 8p21. Biochem. Biophys. Res. Commun. 271: 8-14, 2000.
Sala, C. F.; Formenti, E.; Terstappen, G. C.; Caricasole, A.: Identification, gene structure, and expression of human frizzled-3(FZD3). Biochem. Biophys. Res. Commun. 273: 27-34, 2000.
Chen, H.; Ross, C. A.; Wang, N.; Huo, Y.; MacKinnon, D. F.; Potash, J. B.; Simpson, S. G.; McMahon, F. J.; DePaulo, J. R., Jr.; McInnis, M. G.: NEDD4L on human chromosome 18q21 has multiple forms of transcripts and is a homologue of the mouse Nedd_{—}4-2 gene. Europ. J. Hum. Genet. 9:922-930, 2001.
Erdeniz, N.; Rothstein, R.: Rsp5, a ubiquitin-protein ligase, is involved in degradation of the single-stranded-DNA binding Protein Rfa1 in Saccharomyces cerevisiae. Molec. Cell. Biol. 20: 224-232, 2000.
Bement, W. M.; Wirth, J. A.; Mooseker, M. S.: Cloning and mRNA expression of human unconventional myosin-IC: a homologue of amoeboid myosins-1 with a single IQ motif and an SH3 domain. J. Molec. Biol. 243:356-363, 1994.
Dry, K.; Kenwrick, S.; Rosenthal, A.; Platzer, M.: The complete sequence of the human locus for NgCAM-related cell adhesion molecule reveals a novel alternative exon in chick and man and conserved genomic organization for the L1 sub family. Gene 273: 115-122, 2001.
Grumet, M.; Mauro, V.; Burgoon, M. P.; Edelman, G. M.; Cunningham, B. A.: Structure of a new nervous system glyco protein, Nr-CAM, and its relationship to subgroups of neural cell adhesion molecules. J. Cell. Biol. 113: 1399-1412, 1991
Kayyem, J. F.; Roman, J. M.; de la Rosa, E. J.; Schwarz, U.; Dreyer, W. J.: Bravo/Nr-CAM is closely related to the cell adhesion molecules L1 and Ng-CAM and has a similar heterodimer structure. J. Cell. Biol. 118:1259-1270, 1992.
Lane, R. P.; Chen, X.-N.; Yamakawa, K.; Vielmetter, J.; Korenberg, J. R.; Dreyer, W. J.: Characterization of a highly conserved human homolog to the chicken neural cell surface protein Bravo/Nr-CAM that maps to chromosome band 7q31. Genomics 35: 456-465, 1996.
Wang, B.; Williams, H.; Du, J.-S.; Terrett, J.; Kenwrick, S.: Alternative splicing of human NrCAM in neural and nonneural tissues. Molec. Cell. Neurosci. 10: 287-295, 1998.
Damen, J. E.; Liu, L.; Rosten, P.; Humphries, R. K.; Jefferson, A. B.; Majerus, P. W.; Krystal, G.: The 145-kDa protein induced to associate with Shc by multiple cytokines is an inositol tetraphosphate and phosphatidylinositol 3,4,5-triphosphate 5-phosphatase. Proc. Nat. Acad. Sci. 93: 1689-1693, 1996.
Drayer, A. L.; Pesesse, X.; De Smedt, F.; Woscholski, R.; Parker, P.; Erneux, C.: Cloning and expression of a human placenta inositol 1,3,4,5-tetrakis phosphate and phosphatidylinositol 3,4,5-triphosphate5-phosphatase. Biochem. Biophys. Res. Commun. 225: 243-249, 1996.
Helgason, C. D.; Damen, J. E.; Rosten, P.; Grewal, R.; Sorensen, P.; Chappel, S. M.; Borowski, A.; Jirik, F.; Krystal, G.; Humphries, R. K.: Targeted disruption of SHIP leads to hemopoietic perturbations, lung pathology, and a shortened life span. Genes Dev. 12: 1610-1620, 1998.
Huber, M.; Helgason, C. D.; Damen, J. E.; Liu, L.; Humphries, R. K.; Krystal, G.: The src homology 2-containing inositol phosphatase (SHIP) is the gatekeeper of mast cell degranulation. Proc. Nat. Acad. Sci. 95: 11330-11335, 1998.
Kavanaugh, W. M.; Pot, D. A.; Chin, S. M.; Deuter-Reinhard, M.; Jefferson, A. B.; Norris, F. A.; Masiarz, F. R.; Cousens, L. S.; Majerus, P. W.; Williams, L. T.: Multiple forms of an inositol polyphosphate5-phosphatase form signaling complexes with Shc and Grb2. Curr. Biol. 6: 438-445, 1996.
Lioubin, M. N.; Algate, P. A.; Tsai, S.; Carlberg, K.; Aebersold, R.; Rohrschneider, L. R.: p150(Ship), a signal transduction molecule with inositol polyphosphate-5-phosphatase activity. Genes Dev. 10:1084-1095, 1996.
Liu, Q.; Amgen EST Program; Dumont, D. J.: Molecular cloning and chromosomal localization in human and mouse of the SH2-containing inositol phosphatase, INPP5D (SHIP). Genomics 39: 109-112, 1997.
Liu, Q.; Shalaby, F.; Jones, J.; Bouchard, D.; Dumont, D. J.: The SH2-containing inositol polyphosphate 5-phosphatase, Ship, is expressed during hematopoiesis and spermatogenesis. Blood 91: 2753-2759, 1998.
Takeshita, S.; Namba, N.; Zhao, J. J.; Jiang, Y.; Genant, H. K.; Silva, M. J.; Brodt, M. D.; Helgason, C. D.; Kalesnikoff, J.; Rauh, M. J.; Humphries, R. K.; Krystal, G.; Teitelbaum, S. L.; Ross, F. P.: SHIP-deficient mice are severely osteoporotic due to increased numbers of hyper-resorptive osteoclasts. Nature Med. 8: 943-949, 2002.
Wang, J.-W.; Howson, J. M.; Ghansah, T.; Desponts, C.; Ninos, J. M.; May, S. L.; Nguyen, K. H. T.; Toyama-Sorimachi, N.; Kerr, W. G.: Influence of SHIP on the NK repertoire and allogeneic bone marrow transplantation. Science 295: 2094-2097, 2002.
Ware, M. D.; Rosten, P.; Damen, J. E.; Liu, L.; Humphries, R. K.; Krystal, G.: Cloning and characterization of human SHIP, the 145-kD inositol 5-phosphatase that associates with SHC after cytokine stimulation. Blood 88: 2833-2840, 1996.
Stegmaier, K.; Pendse, S.; Barker, G. F.; Bray-Ward, P.; Ward, D. C.; Montgomery, K. T.; Krauter, K. S.; Reynolds, C.; Sklar, J.; Donnelly, M.; Bohlander, S. K.; Rowley, J. D.; Sallan, S. E.; Gilliland, D. G.; Golub, T. R.: Frequent loss of heterozygosity at the TEL gene locus in acute lymphoblastic leukemia of childhood. Blood 86: 38-44, 1995.
Chen, C.-F.; Chen, Y.; Dai, K.; Chen, P.-L.; Riley, D. J.; Lee, W.-H.: A new member of the hsp90 family of molecular chaperones interacts with the retinoblastoma protein during mitosis and after heat shock. Molec. Cell. Biol. 16: 4691-4699, 1996.
Felts, S. J.; Owen, B. A. L.; Nguyen, P.; Trepel, J.; Donner, D. B.; Toft, D. O.: The hsp90-related protein TRAP1 is a mitochondrial protein with distinct functional properties. J. Biol. Chem. 275:3305-3312, 2000.
Song, H. Y.; Dunbar, J. D.; Zhang, Y. X.; Guo, D.; Donner, D. B.: Identification of a protein with homology to hsp90 that binds the type 1 tumor necrosis factor receptor. J. Biol. Chem. 270: 3574-3581, 1995.
Bates, E. E. M.; Dieu, M.-C.; Ravel, O.; Zurawski, S. M.; Patel, S.; Bridon, J.-M.; Ait-Yahia, S.; Vega, F., Jr.; Banchereau, J.; Lebecque, S.: CD40L activation of dendritic cells down-regulates DORA, a novel member of the immunoglobulin super family. Molec. Immun. 35: 513-524, 1998.
Bates, E. E. M.; Kissenpfennig, A.; Peronne, C.; Mattei, M.-G.; Fossiez, F.; Malissen, B.; Lebecque, S.: The mouse and human IGSF6(DORA) genes map to the inflammatory bowel disease 1 locus and are embedded in an intron of a gene of unknown function. Immunogenetics 52:112-120, 2000.
Koesters, R.; Adams, V.; Betts, D.; Moos, R.; Schmid, M.; Siermann, A.; Hassam, S.; Weitz, S.; Lichter, P.; Heitz, P. U.; von Knebel Doeberitz, M.; Briner, J.: Human eukaryotic initiation factor EIF2C1 gene: cDNA sequence, genomic organization, localization to chromosomal bands 1q34-p35, and expression. Genomics 61: 210-218, 1999.
Martinez, J.; Patkaniowska, A.; Urlaub, H.; Luhrmann, R.; Tuschi, T.: Single-stranded antisense siRNAs guide target RNA cleavage in RNAi. Cell 110: 563-574, 2002.
Hanaoka, E.; Ozaki, T.; Ohira, M.; Nakamura, Y.; Suzuki, M.; Takahashi, E.; Moriya, H.; Nakagawara, A.; Sakiyama, S.: Molecular cloning and expression analysis of the human DA41 gene and its mapping to chromosome 9q21.2-q21.3. J. Hum. Genet. 45: 188-191, 2000.
Ozaki, T.; Hishiki, T.; Toyama, Y.; Yuasa, S.; Nakagawara, A.; Sakiyama, S.: Identification of a new cellular protein that can interact specifically with DAN. DNA Cell Biol. 16: 985-991, 1997.
Akhmanova, A.; Hoogenraad, C. C.; Drabek, K.; Stepanova, T.; Dortland, B.; Verkerk, T.; Vermeulen, W.; Burgering, B. M.; De Zeeuw, C. I.; Grosveld, F.; Galjart, N.: CLASPs are CLIP-115 and -170 associating proteins involved in the regional regulation of microtubule dynamics in motile fibroblasts. Cell 104: 923-935, 2001.
Cattanach, B. M.; Barr, J. A.; Beechey, C. V.; Martin, J.; Noebels, J.; Jones, J.: A candidate model for Angelman syndrome in the mouse. Mammalian Genome 8: 472-478, 1997.
Dhar, M.; Webb, L. S.; Smith, L.; Hauser, L.; Johnson, D.; West, D. B.: A novel ATPase on mouse chromosome 7 is a candidate gene for increased body fat. Physiol. Genomics 4: 93-100, 2000.
Halleck, M. S.; Lawler, J. F., Jr.; Blackshaw, S.; Gao, L.; Nagarajan, P.; Hacker, C.; Pyle, S.; Newman, J. T.; Nakanishi, Y.; Ando, H.; Weinstock, D.; Williamson, P.; Schlegel, R. A.: Differential expression of putative transbilayer amphipath transporters. Physiol. Genomics 1:139-150, 1999.
Loftus, B. J.; Kim, U.-J.; Sneddon, V. P.; Kalush, F.; Brandon, R.; Fuhrmann, J.; Mason, T.; Crosby, M. L.; Barnstead, M.; Cronin, L.; Mays, A. D.; Cao, Y.; Xu, R. X.; Kang, H.-L.; Mitchell, S.; Eichler, E. E.; Harris, P. C.; Venter, J. C.; Adams, M. D.: Genome duplications and other features in 12 Mb of DNA sequence from human chromosome 16p and 16q. Genomics 60: 295-308, 1999.
Asada, H.; Kawamura, Y.; Maruyama, K.; Kume, H.; Ding, R.-G.; Kanbara, N.; Kuzume, H.; Sanbo, M.; Yagi, T.; Obata, K.: Cleft palate and decreased brain gamma-aminobutyric acid in mice lacking the 67-kDa isoform of glutamic acid decarboxylase. Proc. Nat. Acad. Sci. 94:6496-6499, 1997.
Brilliant, M. H.; Szabo, G.; Katarova, Z.; Kozak, C. A.; Glaser, T. M.; Greenspan, R. J.; Housman, D. E.: Sequences homologous to glutamic acid decarboxylase cDNA are present on mouse chromosomes 2 and 10. Genomics 6: 115-122, 1990.
Bu, D.-F.; Tobin, A. J.: The exon-intron organization of the genes (GAD1 and GAD2) encoding two human glutamate decarboxylases (GAD-67 and GAD-65) suggests that they derive from a common ancestral GAD. Genomics 21:222-228, 1994.
Condie, B. G.; Bain, G.; Gottlieb, D. I.; Capecchi, M. R.: Cleft palate in mice with a targeted mutation in the gamma-aminobutyric acid-producing enzyme glutamic acid decarboxylase 67. Proc. Nat. Acad. Sci. 94: 11451-11455, 1997.
Erlander, M. G.; Tillakaratne, N. J. K.; Feldblum, S.; Patel, N.; Tobin, A. J.: Two genes encode distinct glutamate decarboxylases. Neuron 7:91-100, 1991.
Kelly, C. D.; Edwards, Y.; Johnstone, A. P.; Harfst, E.; Nogradi, A.; Nussey, S. S.; Povey, S.; Carter, N. D.: Nucleotide sequence and chromosomal assignment of a cDNA encoding the large isoform of human glutamate decarboxylase. Ann. Hum. Genet. 56: 255-265, 1992.
Krishnamoorthy, K. S.: Pyridoxine-dependency seizure: report of a rare presentation. Ann. Neurol. 13: 103-104, 1983.
Sparkes, R. S.; Kaufman, D. L.; Heinzmann, C.; Tobin, A. J.; Mohandas, T.: Brain glutamate decarboxylase (GAD) gene assigned to human chromosome 2 by somatic cell hybrid analysis. (Abstract) Cytogenet. Cell Genet. 46:696 only, 1987.
Charng, M.-J.; Zhang, D.; Kinnunen, P.; Schneider, M. D.: A novel protein distinguishes between quiescent and activated forms of the type I transforming growth factor beta receptor. J. Biol. Chem. 273:9365-9368, 1998.
Wurthner, J. U.; Frank, D. B.; Felici, A.; Green, H. M.; Cao, Z.; Schneider, M. D.; McNally, J. G.; Lechleider, R. J.; Roberts, A. B.: Transforming growth factor-beta receptor-associated protein 1 is a Smad4 chaperone. J. Biol. Chem. 276: 19495-19502, 2001.
Nagase, T.; Nakayama, M.; Nakajima, D.; Kikuno, R.; Ohara, O.: Prediction of the coding sequences of unidentified human genes. XX. The complete sequences of 100 new cDNA clones from brain which code for large proteins in vitro. DNA Res. 8: 85-95, 2001.
Toda, T.; Iida, A.; Miwa, T.; Nakamura, Y.; Imai, T.: Isolation and characterization of a novel gene encoding nuclear protein at a locus (D11S636) tightly linked to multiple endocrine neoplasia type 1 (MEN1). Hum. Molec. Genet. 3: 465-470, 1994.
Spicer, A. P.; Augustine, M. L.; McDonald, J. A.: Molecular cloning and characterization of a putative mouse hyaluronan synthase. J. Biol. Chem. 271: 23400-23406, 1996.
Faure, S.; Meyer, L.; Costagliola, D.; Vaneensberghe, C.; Genin, E.; Autran, B.; French ALT and IMMUNOCO Study Groups; Delfraisay, J.-F.; SEROCO Study Group; McDermott, D. H.; Murphy, P. M.; Debre, P.; Theodorou, I.; Cambadiere, C.: Rapid progression to AIDS in HIV+ individuals with a structural variant of the chemokine receptor CX(3)CR1. Science 287:2274-2277, 2000.
Imai, T.; Hieshima, K.; Haskell, C.; Baba, M.; Nagira, M.; Nishimura, M.; Kakizaki, M.; Takagi, S.; Nomiyama, H.; Schall, T. J.; Yoshie, O.: Identification and molecular characterization of fractalkine receptor CX3CR1, which mediates both leukocyte migration and adhesion. Cell 91:521-530, 1997.
Moatti, D.; Faure, S.; Fumeron, F.; Amara, M. E. W.; Seknadji, P.; McDermott, D. H.; Debre, P.; Aumont, M. C.; Murphy, P. M.; de Prost, D.; Combadiere, C.: Polymorphism in the fractalkine receptor CX3CR1 as a genetic risk factor for coronary artery disease. Blood 97:1925-1928, 2001.
Raport, C. J.; Schweickart, V. L.; Eddy, R. L., Jr.; Shows, T. B.; Gray, P. W.: The orphan G-protein-coupled receptor-encoding gene V28 is closely related to genes for chemokine receptors and is expressed in lymphoid and neural tissues. Gene 163: 295-299, 1995.
Tripp, R. A.; Jones, L. P.; Haynes, L. M.; Zheng, H.; Murphy, P. M.; Anderson, L. J.: CX3C chemokine mimicry by respiratory syncytial virus G glyco protein. Nature Immun. 2: 732-738, 2001.
Park, W. S.; Lee, J. H.; Shin, M. S.; Park, J. Y.; Kim, H. S.; Lee, J. H.; Kim, Y. S.; Lee, S. N.; Xiao, W.; Park, C. H.; Lee, S. H.; Yoo, N. J.; Lee, J. Y.: Inactivating mutations of the caspase-10 gene in gastric cancer. Oncogene 21: 2919-2925, 2002.
Shin, M. S.; Kim, H. S.; Kang, C. S.; Park, W. S.; Kim, S. Y.; Lee, S. N.; Lee, J. H.; Park, J. Y.; Jang, J. J.; Kim, C. W.; Kim, S. H.; Lee, J. Y.; Yoo, N. J.; Lee, S. H.: Inactivating mutations of CASP10 gene in non-Hodgkin lymphomas. Blood 99: 4094-4099, 2002.
Vincenz, C.; Dixit, V. M.: Fas-associated death domain protein interleukin-1-beta-converting enzyme 2 (FLICE2), an ICE/Ced-3 homologue, is proximally involved in CD95- and p55-mediated death signaling. J. Biol. Chem. 272: 6578-6583, 1997.
Wang, J.; Chun, H. J.; Wong, W.; Spencer, D. M.; Lenardo, M. J.: Caspase-10 is an initiator caspase in death receptor signaling. Proc. Nat. Acad. Sci. 98: 13884-13888, 2001.
Wang, J.; Zheng, L.; Lobito, A.; Chan, F. K.; Dale, J.; Sneller, M.; Yao, X.; Puck, J. M.; Straus, S. E.; Lenardo, M. J.: Inherited human caspase 10 mutations underlie defective lymphocyte and dendritic cell apoptosis in autoimmune lymphoproliferative syndrome type II. Cell 98:47-58, 1999.
Nagase, T.; Kikuno, R.; Nakayama, M.; Hirosawa, M.; Ohara, O.: Prediction of the coding sequences of unidentified human genes. XVIII. The complete sequences of 100 new cDNA clones from brain which code for large proteins in vitro. DNA Res. 7: 273-281, 2000.
Nagase, T.; Ishikawa, K.; Miyajima, N.; Tanaka, A.; Kotani, H.; Nomura, N.; Ohara, O.: Prediction of the coding sequences of unidentified human genes. IX. The complete sequences of 100 new cDNA clones from brain which can code for large proteins in vitro. DNA Res. 5: 31-39, 1998.
Herzing, L. B. K.; Kim, S.-J.; Cook, E. H., Jr.; Ledbetter, D. H.: The human aminophospholipid-transporting ATPase gene ATP10C maps adjacent to UBE3A and exhibits similar imprinted expression. Am. J. Hum. Genet. 68: 1501-1505, 2001.
Dunlevy, J. R.; Berryhill, B. L.; Vergnes, J.-P.; SundarRaj, N.; Hassell, J. R.: Cloning, chromosomal localization, and characterization of cDNA from a novel gene, SH3BP4, expressed by human corneal fibroblasts. Genomics 62:519-524, 1999.
Wong, W. T.; Schumacher, C.; Salcini, A. E.; Romano, A.; Castagnino, P.; Pelicci, P. G.; DiFiore, P. P.: A protein-binding domain, EH, identified in the receptor tyrosine kinase substrate Eps15 and conserved in evolution. Proc. Nat. Acad. Sci. 92: 9530-9534, 1995.
Meguro, M.; Kashiwagi, A.; Mitsuya, K.; Nakao, M.; Kondo, I.; Saitoh, S.; Oshimura, M.: A novel maternally expressed gene, ATP10C, encodes a putative aminophospholipid translocase associated with Angelman syndrome. Nature Genet. 28: 19-20, 2001.
Hampe, J.; Grebe, J.; Nikolaus, S.; Solberg, C.; Croucher, P. J. P.; Mascheretti, S.; Jahnsen, J.; Moum, B.; Klump, B.; Krawczak, M.; Mirza, M. M.; Foelsch, U. R.; Vatn, M.; Schreiber, S.: Association of NOD2 (CARD 15) genotype with clinical course of Crohn's disease: a cohort study. Lancet 359: 1661-1665, 2002.
Hugot, J.-P.; Chamaillard, M.; Zouali, H.; Lesage, S.; Cezard, J.-P.; Belaiche, J.; Almer, S.; Tysk, C.; O'Morain, C. A.; Gassull, M.; Binder, V.; Finkel, Y.; and 8 others: Association of NOD2 leucine-rich repeat variants with susceptibility to Crohn's disease. Nature 411:599-603, 2001.
Murillo, L.; Crusius, J. B. A.; van Bodegraven, A. A.; Alizadeh, B. Z.; Pena, A. S.: CARD15 gene and the classification of Crohn's disease. Immunogenetics 54: 59-61, 2002.
Ogura, Y.; Bonen, D. K.; Inohara, N.; Nicolae, D. L.; Chen, F. F.; Ramos, R.; Britton, H.; Moran, T.; Karaliuskas, R.; Duerr, R. H.; Achkar, J.-P.; Brant, S. R.; Bayless, T. M.; Kirschner, B. S.; Hanauer, S. B.; Nunez, G.; Cho, J. H.: A frameshift mutation in Nod2 associated with susceptibility to Crohn's disease. Nature 411: 603-606, 2001.
Ogura, Y.; Inohara, N.; Benito, A.; Chen, F. F.; Yamaoka, S.; Nunez, G.: Nod2, a Nod1/Apaf-1 family member that is restricted to monocytes and activates NF-kappa-B. J. Biol. Chem. 276: 4812-4818, 2001.
Lesage, S.; Zouali, H.; Cezard, J.-P.; EPWG-IBD Group; Colombel, J.-F.; EPIMAD Group; Belaiche, J.; GETAID Group; Almer, S.; Tysk, C.; O'Morain, C.; Gassull, M.; Binder, V.; Finkel, Y.; Modigliani, R.; Gower-Rousseau, C.; Macry, J.; Merlin, F.; Chamaillard, M.; Jannot, A.-S.; Thomas, G.; Hugot, J.-P.: CARD15/NOD2 mutational analysis and genotype-phenotype correlation in 612 patients with inflammatory bowel disease. Am. J. Hum. Genet. 70: 845-857, 2002.
van Heel, D. A.; McGovern, D. P. B.; Cardon, L. R.; Dechairo, B. M.; Lench, N. J.; Carey, A. H.; Jewell, D. P.: Fine mapping of the IBD1 locus did not identify Crohn disease-associated NOD2 variants: implications for complex disease genetics. Am. J. Med. Genet. 111:253-259, 2002.
Vermeire, S.; Wild, G.; Kocher, K.; Cousineau, J.; Dufresne, L.; Bitton, A.; Langelier, D.; Pare, P.; Lapointe, G.; Cohen, A.; Daly, M. J.; Rioux, J. D.: CARD15 genetic variation in a Quebec population: prevalence, genotype-phenotype relationship, and haplotype structure. Am. J. Hum. Genet. 71: 74-83, 2002.
Yamazaki, K.; Takazoe, M.; Tanaka, T.; Kazumori, T.; Nakamura, Y.: Absence of mutation in the NOD2/CARD15 gene among 483 Japanese patients with Crohn's disease. J. Hum. Genet. 47: 469-472, 2002.
Chong, S. S.; Tanigami, A.; Roschke, A. V.; Ledbetter, D. H.: 14-3-3-epsilon has no homology to LIS1 and lies telomeric to it on chromosome 17p13.3 outside the Miller-Dieker syndrome chromosome region. Genome Res. 6: 735-741, 1996.
Conklin, D. S.; Galaktionov, K.; Beach, D.: 14-3-3 proteins associate with cdc25 phosphatases. Proc. Nat. Acad. Sci. 92: 7892-7896, 1995.
Jin, D.-Y.; Lyu, M. S.; Kozak, C. A.; Jeang, K.-T.: Function of 14-3-3 proteins. Nature 382: 308 only, 1996.
Luk, S. C. W.; Garcia-Barcelo, M.; Tsui, S. K. W.; Fung, K. P.; Lee, C. Y.; Waye, M. M. Y.: Assignment of the human 14-3-3 epsilon isoform (YWHAE) to human chromosome 17p13 by in situ hybridization. Cytogenet. Cell Genet. 78: 105-106, 1997
Slentz-Kesler, K.; Moore, J. T.; Lombard, M.; Zhang, J.; Hollingsworth, R.; Weiner, M. P.: Identification of the human Mnk2 gene (MKNK2) through protein interaction with estrogen receptor beta. Genomics 69:63-71, 2000.
Li, Y.; He, X.; Schembri-King, J.; Jakes, S.; Hayashi, J.: Cloning and characterization of human Lnk, an adaptor protein with pleckstrin homology and Src homology 2 domains that can inhibit T cell activation. J. Immun. 164: 5199-5206, 2000.
Takaki, S.; Sauer, K.; Iritani, B. M.; Chien, S.; Ebihara, Y.; Tsuji, K.; Takatsu, K.; Perlmutter, R. M.: Control of B cell production by the adaptor protein Lnk: definition of a conserved family of signal-modulating proteins. Immunity 13: 599-609, 2000.
Burmeister, M.; Meyer, G. E.: The trefoil gene maps to mouse chromosome 17. Mammalian Genome 8: 223-224, 1997.
Chinery, R.; Williamson, J.; Poulsom, R.: The gene encoding human intestinal trefoil factor (TFF3) is located on chromosome 21q22.3 clustered with other members of the trefoil peptide family. Genomics 32:281-284, 1996.
Mashimo, H.; Wu, D.-C.; Podolsky, D. K.; Fishman, M. C.: Impaired defense of intestinal mucosa in mice lacking intestinal trefoil factor. Science 274:262-265, 1996.
Podolsky, D. K.; Lynch-Devaney, K.; Stow, J. L.; Oates, P.; Murgue, B.; DeBeaumont, M.; Sands, B. E.; Mahida, Y. R.: Identification of human intestinal trefoil factor: goblet cell-specific expression of a peptide targeted for apical secretion. J. Biol. Chem. 268: 6694-6702, 1993.
Probst, J. C.; Zetzsche, T.; Weber, M.; Theilemann, P.; Skutella, T.; Landgraf, R.; Jirikowski, G. F.: Human intestinal trefoil factor is expressed in human hypothalamus and pituitary: evidence for a novel neuropeptide. FASEBJ. 10: 1518-1523, 1996.
Schmitt, H.; Wundrack, I.; Beck, S.; Gott, P.; Welter, C.; Shizuya, H.; Simon, M. I.; Blin, N.: A third P-domain peptide gene (TFF3), human intestinal trefoil factor, maps to 21q22.3. Cytogenet. Cell Genet. 72: 299-302, 1996.
Taupin, D.; Wu, D.-C.; Jeon, W.-K.; Devaney, K.; Wang, T. C.; Podolsky, D. K.: The trefoil gene family are coordinately expressed immediate-early genes: EGF receptor- and MAP kinase-dependent interregulation. J. Clin. Invest. 103: R31-R38, 1999.
Thim, L.: A new family of growth factor-like peptides: ‘tre-foil’ disulphide loop structures as a common feature in breast cancer associated peptide (pS2), pancreatic spasmolytic polypeptide (PSP), and frogskin peptides (spasmolysins). FEBS Lett. 250: 85-90, 1989.
England, S. K.; Uebele, V. N.; Kodali, J.; Bennett, P. B.; Tamkun, M. M.: A novel K+ channel beta-subunit (hKv-beta-1.3) is produced via alternative mRNA splicing. J. Biol. Chem. 270: 28531-28534, 1995.
Aubry, F.; Mattei, M.-G.; Barque, J.-P.; Galibert, F.: Chromosomal localization and expression pattern of the RNase L inhibitor gene. FEBS Lett. 381: 135-139, 1996.
Diriong, S.; Salehzada, T.; Bisbal, C.; Martin and, C.; Taviaux, S.: Localization of the ribonuclease L inhibitor gene (RNS41), anew member of the interferon-regulated 2-5A pathway, to 4q31 by fluorescence in situ hybridization. Genomics 32: 488-490, 1996.
Carson-Walter, E. B.; Watkins, D. N.; Nanda, A.; Vogelstein, B.; Kinzler, K. W.; St. Croix, B.: Cell surface tumor endothelial markers are conserved in mice and humans. Cancer Res. 61: 6649-6655, 2001.
Pizzuti, A.; Amati, F.; Calabrese, G.; Mari, A.; Colosimo, A; Silani, V.; Giardino, L.; Ratti, A.; Penso, D.; Calza, L.; Palka, G.; Scarlato, G.; Novelli, G.; Dallapicolla, B.: cDNA characterization and chromosomal mapping of two human homologs of the Drosophila dishevelled polarity gene. Hum. Molec. Genet. 5: 953-958, 1996.
Semenov, M. V.; Snyder, M.: Human dishevelled genes constitute a DHR-containing multi gene family. Genomics 42: 302-310, 1997.
St. Croix, B.; Rago, C.; Velculescu, V.; Traverso, G.; Romans, K. E.; Montgomery, E.; Lal, A.; Riggins, G. J.; Lengauer, C.; Vogelstein, B.; Kinzler, K. W.: Genes expressed in human tumor endothelium. Science 289:1197-1202, 2000.
Gosling, J.; Dairaghi, D. J.; Wang, Y.; Hanley, M.; Talbot, D.; Miao, Z.; Schall, T. J.: Cutting edge: identification of a novel chemokine receptor that binds dendritic cell- and T cell-active chemokines including ELC, SLC, and TECK. J. Immun. 164: 2851-2856, 2000.
Hadjantonakis, A.-K.; Formstone, C. J.; Little, P. F. R.: mCelsr1 is an evolutionarily conserved seven-pass transmembrane receptor and is expressed during mouse embryonic development. Mech. Dev. 78:91-95, 1998.
Hadjantonakis, A.-K.; Sheward, W. J.; Harmar, A. J.; de Galan, L.; Hoovers, J. M. N.; Little, P. F. R.: Celsr1, a neural-specific gene encoding an unusual seven-pass transmembrane receptor, maps to mouse chromosome 15 and human chromosome 22qter. Genomics 45: 97-104, 1997
Chai, J.; Du, C.; Wu, J.-W.; Kyin, S.; Wang, X.; Shi, Y.: Structural and biochemical basis of apoptotic activation by Smac/DIABLO. Nature 406:855-862, 2000.
Du, C.; Fang, M.; Li, Y.; Li, L.; Wang, X.: Smac, a mitochondrial protein that promotes cytochrome c-dependent caspase activation by eliminating IAP inhibition. Cell 102: 33-42, 2000.
Okada, H.; Suh, W.-K.; Jin, J.; Woo, M.; Du, C.; Elia, A.; Duncan, G. S.; Wakeham, A.; Itie, A.; Lowe, S. W.; Wang, X.; Mak, T. W.: Generation and characterization of Smac/DIABLO-deficient mice. Molec. Cell. Biol. 22: 3509-3517, 2002.
Scott, A. F.: Personal Communication. Baltimore, Md. Aug. 18, 2000.
Verhagen, A. M.; Ekert, P. G.; Pakusch, M.; Silke, J.; Connolly, L. M.; Reid, G. E.; Moritz, R. L.; Simpson, R. J.; Vaux, D. L.: Identification of DIABLO, a mammalian protein that promotes apoptosis by binding to and antagonizing IAP proteins. Cell 102: 43-53, 2000.
Bousquet, O.; Basseville, M.; Vila-Porcile, E.; Billette de Villemeur, T.; Hauw, J.-J.; Landrieu, P.; Portier, M.-M.: Aggregation of a subpopulation of vimentin filaments in cultured human skin fibroblasts derived from patients with giant axonal neuropathy. Cell. Motil. Cytoskeleton 33:115-129, 1996.
Kuhlenbaumer, G.; Young, P.; Oberwittler, C.; Hunermund, G.; Schirmacher, A.; Domschke, K.; Ringelstein, B.; Stogbauer, F.: Giant axonal neuropathy (GAN): case report and two novel mutations in the gigaxonin gene. Neurology 58:1273-1276, 2002. Note: Erratum: Neurology 58: 1444, 2002.
Pena, S. D.: Giant axonal neuropathy: an inborn error of organization of intermediate filaments. Muscle Nerve 5: 166-172, 1982.
Prineas, J. W.; Ouvrier, R. A.; Wright, R. G.; Walsh, J. C.; McLeod, J. G.: Giant axonal neuropathy: a generalized disorder of cytoplasmic microfilament formation. J. Neuropath. Exp. Neurol. 35: 458-470, 1976.
Scott, A. F.: Personal Communication. Baltimore, Md. Mar. 13, 2001.
Mansharamani, M.; Hewetson, A.; Chilton, B. S.: Cloning and characterization of an atypical type IV P-type ATPase that binds to the RING motif of RUSH transcription factors. J. Biol. Chem. 276: 3641-3649, 2001.
Bullrich, F.; Druck, T.; Kunapuli, P.; Gomez, J.; Gripp, K. W.; Schlegelberger, B.; Lasota, J.; Aronson, M.; Cannizzaro, L. A.; Huebner, K.; Benovic, J. L.: Chromosomal mapping of the genes GPRK5 and GPRK6 encoding G protein-coupled receptor kinases GRK5 and GRK6. Cytogenet. Cell Genet. 70: 250-254, 1995.
Haribabu, B.; Snyderman, R.: Identification of additional members of human G-protein-coupled receptor kinase multi gene family. Proc. Nat. Acad. Sci. 90: 9398-9402, 1993.
Hasson, T.; Skowron, J. F.; Gilbert, D. J.; Avraham, K. B.; Perry, W. L.; Bement, W. M.; Anderson, B. L.; Sherr, E. H.; Chen, Z.-Y.; Greene, L. A.; Ward, D. C.; Corey, D. P.; Mooseker, M. S.; Copeland, N. G.; Jenkins, N. A.: Mapping of unconventional myosins in mouse and human. Genomics 36: 431-439, 1996.
Laporte, J.; Hu, L. J.; Kretz, C.; Mandel, J.-L.; Kioschis, P.; Coy, J. F.; Klauck, S. M.; Poustka, A.; Dahl, N.: A gene mutated in X-linked myotubular myopathy defines a new putative tyrosine phosphatase family conserved in yeast. Nature Genet. 13: 175-182, 1996.
Chambost, H.; Van Baren, N.; Brasseur, F.; Godelaine, D.; Xerri, L.; Landi, S. J.; Theate, I.; Plumas, J.; Spagnoli, G. C.; Michel, G.; Coulie, P. G.; Olive, D.: Expression of gene MAGE-A4 in Reed-Sternberg cells. Blood 95: 3530-3533, 2000.
Offenberg, H. H.; Schalk, J. A. C.; Meuwissen, R. L. J.; van Aalderen, M.; Kester, H. A.; Dietrich, A. J. J.; Heyting, C.: SCP2: a major protein component of the axial elements of synaptonemal complexes of the rat. Nucleic Acids Res. 26: 2572-2579, 1998.
Schalk, J. A. C.; Offenberg, H. H.; Peters, E.; Groot, N. P. B.; Hoovers, J. M. N.; Heyting, C.: Isolation and characterization of the human SCP2 cDNA and chromosomal localization of the gene. Mammalian Genome 10: 642-644, 1999.
Hatamura, I.; Kanauchi, Y.; Takahara, M.; Fujiwara, M.; Muragaki, Y.; Ooshima, A.; Ogino, T.: A nonsense mutation in TRPS1 in a Japanese family with tricho-rhino-phalangeal syndrome type I. (Letter) Clin. Genet. 59: 366-367, 2001.
Hilton, M. J.; Sawyer, J. M.; Gutierrez, L.; Hogart, A.; Kung, T. C.; Wells, D. E.: Analysis of novel and recurrent mutations responsible for the tricho-rhino-phalangeal syndromes. J. Hum. Genet. 47: 103-106, 2002.
Schinkmann, K.; Blenis, J.: Cloning and characterization of a human STE20-like protein kinase with unusual cofactor requirements. J. Biol. Chem. 272: 28695-28703, 1997.
Zhou, T.-H.; Ling, K.; Guo, J.; Zhou, H.; Wu, Y.-L.; Jing, Q.; Ma, L.; Pei, G.: Identification of a human brain-specific isoform of mammalian STE20-like kinase 3 that is regulated by cAMP-dependent protein kinase. J. Biol. Chem. 275: 2513-2519, 2000.
Ono, Y.; Ohno, M.; Shimura, Y.: Identification of a putative RNA helicase (HRH1), a human homolog of yeast Prp22. Molec. Cell. Biol. 14:7611-7620, 1994.
Wang, Y.; Cortez, D.; Yazdi, P.; Neff, N.; Elledge, S. J.; Qin, J.: BASC, a super complex of BRCA1-associated proteins involved in the recognition and repair of aberrant DNA structures. Genes Dev. 14:927-939, 2000.
Thim, L.; Woldike, H. F.; Nielsen, P. F.; Christensen, M.; Lynch-Devaney, K.; Podolsky, D. K.: Characterization of human and rat intestinal trefoil factor produced in yeast. Biochemistry 34: 4757-4764, 1995.
Kimura, S.; Hara, Y.; Pineau, T.; Fernandez-Salguero, P.; Fox, C. H.; Ward, J. M.; Gonzalez, F. J.: The T/ebp null mouse: thyroid-specific enhancer-binding protein is essential for the organogenesis of the thyroid, lung, ventral forebrain, and pituitary. Genes Dev. 10:60-69, 1996.
Fernandes-Alnemri, T.; Armstrong, R. C.; Krebs, J.; Srinivasula, S. M.; Wang, L.; Bullrich, F.; Fritz, L. C.; Trapani, J. A.; Tomaselli, K. J.; Litwack, G.; Alnemri, E. S.: In vitro activation of CPP32 and Mch3 by Mch4, a novel human apoptotic cysteine protease containing two FADD-like domains. Proc. Nat. Acad. Sci. 93: 7464-7469, 1996.
Levkau, B.; Koyama, H.; Raines, E. W.; Clurman, B. E.; Herren, B.; Orth, K.; Roberts, J. M.; Ross, R.: Cleavage of p21(Cip1/Waf1) and p27(Kip1) mediates apoptosis in endothelial cells through activation of Cdk2: role of a caspase cascade. Molec. Cell 1: 553-563, 1998.
Nasir, J.; Theilmann, J. L.; Chopra, V.; Jones, A. M.; Walker, D.; Rasper, D. M.; Vaillancourt, J. P.; Hewitt, J. E.; Nicholson, D. W.; Hayden, M. R.: Localization of the cell death genes CPP32 and Mch-2 to human chromosome 4q. Mammalian Genome 8: 56-59, 1997.
Tiso, N.; Pallavicini, A.; Muraro, T.; Zimbello, R.; Apolloni, E.; Valle, G.; Lanfranchi, G.; Danieli, G. A.: Chromosomal localization of the human genes, CPP32, Mch2, Mch3, and Ich-1, involved in cellular apoptosis. Biochem. Biophys. Res. Commun. 225: 983-989, 1996.
Hopfner, K.-P.; Karcher, A.; Craig, L.; Woo, T. T.; Carney, J. P.; Tainer, J. A.: Structural biochemistry and interaction architecture of the DNA double-strand break repair Mre111 nuclease and Rad50-ATPase. Cell 105:473-485, 2001.
Stracker, T. H.; Carson, C. T.; Weitzman, M. D.: Adenovirus oncoproteins inactivate the Mre11-Rad50-NBS1 DNA repair complex. Nature 418:348-352, 2002.
Zhong, Q.; Chen, C.-F.; Li, S.; Chen, Y.; Wang, C.-C.; Xiao, J.; Chen, P.-L.; Sharp, Z. D.; Lee, W.-H.: Association of BRCA1 with the hRad50-hMre11-p95 complex and the DNA damage response. Science 285:747-750, 1999.
Zhu, X.-D.; Kuster, B.; Mann, M.; Petrini, J. H. J.; de Lange, T.: Cell-cycle-regulated association of RAD50/MRE11/NBS1 with TRF2 and human telomeres. Nature Genet. 25: 347-352, 2000.
Lin, Q.; Schwarz, J.; Bucana, C.; Olson, E. N.: Control of mouse cardiac morphogenesis and myogenesis by transcription factor MEF2C. Science 276:1404-1407, 1997.
Fernandes-Alnemri, T.; Litwack, G.; Alnemri, E. S.: Mch2, a new member of the apoptotic Ced-3/Ice cysteine protease gene family. Cancer Res. 55: 2737-2742, 1995.
Orth, K.; Chinnaiyan, A. M.; Garg, M.; Froelich, C. J.; Dixit, V. M.: The CED-3/ICE-like protease Mch2 is activated during apoptosis and cleaves the death substrate lamin A. J. Biol. Chem. 271: 16443-16446, 1996.
Verhaegh, G. W. C. T.; Jongmans, W.; Jaspers, N. G. J.; Natarajan, A. T.; Oshimura, M.; Lohman, P. H. M.; Zdzienicka, M. Z.: A gene that regulates DNA replication in response to DNA damage is located on human chromosome 4q. Am. J. Hum. Genet. 57: 1095-1103, 1995.
De Plaen, E.; Arden, K.; Traversari, C.; Gaforio, J. J.; Szikora, J.-P.; De Smet, C.; Brasseur, F.; van der Bruggen, P.; Lethe, B.; Lurquin, C.; Brasseur, R.; Chomez, P.; De Backer, O.; Cavenee, W.; Boon, T.: Structure, chromosomal localization, and expression of 12 genes of the MAGE family. Immunogenetics 40: 360-369, 1994.
Rogner, U. C.; Wilke, K.; Steck, E.; Korn, B.; Poustka, A.: The melanoma antigen gene (MAGE) family is clustered in the chromosomal band Xq28. Genomics 29: 725-731, 1995.
McDermott, J. C.; Cardoso, M. C.; Yu, Y.-T.; Andres, V.; Leifer, D.; Krainc, D.; Lipton, S. A.; Nadal-Ginard, B.: hMEF2C gene encodes skeletal muscle- and brain-specific transcription factors. Molec. Cell. Biol. 13: 2564-2577, 1993.
Emes, R. D.; Ponting, C. P.: A new sequence motif linking lissencephaly, Treacher Collins and oral-facial-digital type 1 syndromes, microtubule dynamics and cell migration. Hum. Molec. Genet. 10: 2813-2820, 2001.
Laporte, J.; Blondeau, F.; Buj-Bello, A.; Tentler, D.; Kretz, C.; Dahl, N.; Mandel, J.-L.: Characterization of the myotubular in dual specificity phosphatase gene family from yeast to human. Hum. Molec. Genet. 7: 1703-1712, 1998.
Borggrefe, T.; Masat, L.; Wabl, M.; Riwar, B.; Cattoretti, G.; Jessberger, R.: Cellular, intracellular, and developmental expression patterns of murine SWAP-70. Europ. J. Immun. 29: 1812-1822, 1999.
Borggrefe, T.; Wabl, M.; Akhmedov, A. T.; Jessberger, R.: A B-cell-specific DNA recombination complex. J. Biol. Chem. 273: 17025-17035, 1998.
Masat, L.; Caldwell, J.; Armstrong, R.; Khoshnevisan, H.; Jessberger, R.; Herndier, B.; Wabl, M.; Ferrick, D.: Association of SWAP-70 with the B cell antigen receptor complex. Proc. Nat. Acad. Sci. 97: 2180-2184, 2000.
Masat, L.; Liddell, R. A.; Mock, B. A.; Kuo, W.-L.; Jessberger, R.; Wabl, M.; Morse, H. C., III: Mapping of the SWAP70 gene to mouse chromosome 7 and human chromosome 11p15. Immunogenetics 51: 16-19, 2000.
Shinohara, M.; Terada, Y.; Iwamatsu, A.; Shinohara, A.; Mochizuki, N.; Higuchi, M.; Gotoh, Y.; Ihara, S.; Nagata, S.; Itoh, H.; Fukui, Y.; Jessberger, R.: SWAP-70 is a guanine-nucleotide-exchange factor that mediates signalling of membrane ruffling. Nature 416: 759-763, 2002.
Kamimoto, T.; Zama, T.; Aoki, R.; Muro, Y.; Hagiwara, M.: Identification of a novel kinesin-related protein, KRMP1, as a target for mitotic peptidyl-prolyl isomerase Pin1. J. Biol. Chem. 276: 37520-37528, 2001.
Ansel, K. M.; Harris, R. B. S.; Cyster, J. G.: CXCL13 is required for B1 cell homing, natural antibody production, and body cavity immunity. Immunity 16:67-76, 2002.
Gunn, M. D.; Ngo, V. N.; Ansel, K. M.; Ekland, E. H.; Cyster, J. G.; Williams, L. T.: A B-cell-homing chemokine made in lymphoid follicles activates Burkitt's lymphoma receptor-1. Nature 391: 799-803, 1998.
Legler, D. F.; Loetscher, M.; Roos, R. S.; Clark-Lewis, I.; Baggiolini, M.; Moser, B.: B cell-attracting chemokine 1, a human CXC chemokine expressed in lymphoid tissues, selectively attracts B lymphocytes via BLR1/CXCR5. J. Exp. Med. 187: 655-660, 1998.
Oh, J.; Takahashi, R.; Kondo, S.; Mizoguchi, A.; Adachi, E.; Sasahara, R. M.; Nishimura, S.; Imamura, Y.; Kitayama, H.; Alexander, D. B.; Ide, C.; Horan, T. P.; Arakawa, T.; Yoshida, H.; Nishikawa, S.; Itoh, Y.; Seiki, M.; Itohara, S.; Takahashi, C.; Noda, M.: The membrane-anchored MMP inhibitor RECK is a key regulator of extracellular matrix integrity and angiogenesis. Cell 107: 789-800, 2001.
Takahashi, C.; Sheng, Z.; Horan, T. P.; Kitayama, H.; Maki, M.; Hitomi, K.; Kitaura, Y.; Takai, S.; Sasahara, R. M.; Horimoto, A.; Ikawa, Y.; Ratzkin, B. J.; Arakawa, T.; Noda, M.: Regulation of matrix metalloproteinase-9 and inhibition of tumor invasion by the membrane-anchored glyco protein RECK. Proc. Nat. Acad. Sci. 95: 13221-13226, 1998.
Nagase, T.; Ishikawa, I.; Nakajima, D.; Ohira, M.; Seki, N.; Miyajima, N.; Tanaka, A.; Kotani, H.; Nomura, N.; O'Hara, O.: Prediction of the coding sequences of unidentified human genes. VII. The complete sequences of 100 new cDNA clones from brain which can code for large proteins in vitro. DNA Res. 4: 141-150, 1997.
Muto, A.; Hoshino, H.; Madisen, L.; Yanai, N.; Obinata, M.; Karasuyama, H.; Hayashi, N.; Nakauchi, H.; Yamamoto, M.; Groudine, M.; Igarashi, K.: Identification of Bach2 as a B-cell-specific partner for small Maf proteins that negatively regulate the immunoglobulin heavy chain gene 3-prime enhancer. EMBO J. 17: 5734-5743, 1998.
Sasaki, S.; Ito, E.; Toki, T.; Maekawa, T.; Kanezaki, R.; Umenai, T.; Muto, A.; Nagai, H.; Kinoshita, T.; Yamamoto, M.; Inazawa, J.; Taketo, M. M.; Nakahata, T.; Igarashi, K.; Yokoyama, M.: Cloning and expression of human B cell specific transcription factor BACH2 mapped to chromosome 6q15. Oncogene 19: 3739-3749, 2000
Amagai, M.; Wang, Y.; Minoshima, S.; Kawamura, K.; Green, K. J.; Nishikawa, T.; Shimizu, N.: Assignment of the human genes for desmocollin3 (DSC3) and desmocollin 4 (DSC4) to chromosome 18q12. Genomics 25:330-332, 1995.
Kawamura, K.; Watanabe, K.; Suzuki, T.; Yamakawa, T.; Kamiyama, T.; Nakagawa, H.; Tsurufuji, S.: cDNA cloning and expression of a novel human desmocollin. J. Biol. Chem. 269: 26295-26302, 1994.
King, I. A.; Sullivan, K. H.; Bennett, R., Jr.; Buxton, R. S.: The desmocollins of human foreskin epidermis: identification and chromosomal assignment of a third gene and expression patterns of the three isoforms. J. Invest. Derm. 105: 314-321, 1995.
Hoop, R. C.; Russo, L. S.; Riconda, D. L.; Schwartz, L. S.; Hoffman, E. P.: Restoration of half the normal dystrophin sequence in a double-deletion Duchenne muscular dystrophy family. Am. J. Med. Genet. 49: 323-327, 1994.
Burger, J.; Fonknechten, N.; Hoeltzenbein, M.; Neumann, L.; Bratanoff, E.; Hazan, J.; Reis, A.: Hereditary spastic paraplegia caused by mutations in the SPG4 gene. Europ. J. Hum. Genet. 8: 771-776, 2000.
Burger, J.; Metzke, H.; Paternotte, C.; Schilling, F.; Hazan, J.; Reis, A.: Autosomal dominant spastic paraplegia with anticipation maps to a 4-cM interval on chromosome 2p21-p24 in a large German family. Hum. Genet. 98: 371-375, 1996.
Errico, A.; Ballabio, A.; Rugarli, E. I.: Spastin, the proteinmutated in autosomal dominant hereditary spastic paraplegia, is involved in microtubule dynamics. Hum. Molec. Genet. 11: 153-163, 2002.
Lindsey, J. C.; Lusher, M. E.; McDermott, C. J.; White, K. D.; Reid, E.; Rubinsztein, D. C.; Bashir, R.; Hazan, J.; Shaw, P. J.; Bushby, K. M. D.: Mutation analysis of the spastin gene (SPG4) inpatients with hereditary spastic paraplegia. J. Med. Genet. 37:759-765, 2000.
Takahashi, N.; Tuiki, H.; Saya, H.; Kaibuchi, K.: Localization of the gene coding for ROCK II/Rho kinase on human chromosome 2p24. Genomics 55:235-237, 1999.
Kedra, D.; Pan, H.-Q.; Seroussi, E.; Fransson, I.; Guilbaud, C.; Collins, J. E.; Dunham, I.; Blennow, E.; Roe, B. A.; Piehl, F.; Dumanski, J. P.: Characterization of the human synaptogyrin gene family. Hum. Genet. 103: 131-141, 1998.
Hazan, J.; Davoine, C. S.; Mavel, D.; Fonknechten, N.; Paternotte, C.; Fizames, C.; Cruaud, C.; Samson, D.; Muselet, D.; Vega-Czarny, N.; Brice, A.; Gyapay, G.; Heilig, R.; Fontaine, B.; Weissenbach, J.: A fine integrated map of the SPG4 locus excludes an expanded CAG repeat in chromosome 2p-linked autosomal dominant spastic paraplegia. Genomics 60:309-319, 1999.
Hazan, J.; Fonknechten, N.; Mavel, D.; Paternotte, C.; Samson, D.; Artiguenave, F.; Davoine, C.-S.; Cruaud, C.; Durr, A.; Wincker, P.; Brottier, P.; Cattolico, L.; Barbe, V.; Burgunder, J.-M.; Prud'homme, J.-F.; Brice, A.; Fontaine, B.; Heilig, R.; Weissenbach, J.: Spastin, a new AAA protein, is altered in the most frequent form of autosomal dominant spastic paraplegia. Nature Genet. 23: 296-303, 1999.
Lee, M. P.; Brandenburg, S.; Landes, G. M.; Adams, M.; Miller, G.; Feinberg, A. P.: Two novel genes in the center of the 11p15 imprinted domain escape genomic imprinting. Hum. Molec. Genet. 8: 683-690, 1999.
Nakayama, M.; Nakajima, D.; Nagase, T.; Nomura, N.; Seki, N.; Ohara, O.: Identification of high-molecular-weight proteins with multiple EGF-like motifs by motif-trap screening. Genomics 51: 27-34, 1998.
Wu, Q.; Maniatis, T.: Large exons encoding multiple ectodomains are a characteristic feature of proto-cadherin genes. Proc. Nat. Acad. Sci. 97: 3124-3129, 2000.
Conklin, D. C.; Rixon, M. W.; Kuestner, R. E.; Maurer, M. F.; Whitmore, T. E.; Millar, R. P.: Cloning and gene expression of a novel human ribonucleoprotein. Biochim. Biophys. Acta 1492: 465-469, 2000.
Kataoka, N.; Yong, J.; Kim, V. N.; Velazquez, F.; Perkinson, R. A.; Wang, F.; Dreyfuss, G.: Pre-mRNA splicing imprints mRNA in the nucleus with a novel RNA-binding protein that persists in the cytoplasm. Molec. Cell 6: 673-682, 2000.
Salicioni, A. M.; Xi. M.; Vanderveer, L. A.; Balsara, B.; Testa, J. R.; Dunbrack, R. L., Jr.; Godwin, A. K.: Identification and structural analysis of human RBM8A and RBM8B: two highly conserved RNA-binding motif proteins that interact with OVCA1, a candidate tumor suppressor. Genomics 69:54-62, 2000.
Zhao, X.-F.; Nowak, N. J.; Shows, T. B.; Aplan, P. D.: MAGOH interacts with a novel RNA-binding protein. Genomics 63: 145-148, 2000.
Caira, F.; Antonson, P.; Pelto-Huikko, M.; Treuter, E.; Gustafsson, J.-A.: Cloning and characterization of RAP250, a novel nuclear receptor coactivator. J. Biol. Chem. 275: 5308-5317, 2000.
Guan, X. Y.; Xu, J.; Anzick, S. L.; Zhang, H.; Trent, J. M.; Meltzer, P. S.: Hybrid selection of transcribed sequences from microdissected DNA: isolation of genes within amplified region at 20q11-q13.2 in breast cancer. Cancer Res. 56: 3446-3450, 1996.
Ko, L.; Cardona, G. R.; Chin, W. W.: Thyroid hormone receptor-binding protein, an LXXLL motif-containing protein, functions as a general coactivator. Proc. Nat. Acad. Sci. 97: 6212-6217, 2000.
Lee, S.-K.; Anzick, S. L.; Choi, J.-E.; Bubendorf, L.; Guan, X.-Y.; Jung, Y.-K.; Kallioniemi, O. P.; Kononen, J.; Trent, J. M.; Azorsa, D.; Jhun, B.-H.; Cheong, J. H.; Lee, Y. C.; Meltzer, P. S.; Lee, J. W.: A nuclear factor, ASC-2, as a cancer-amplified transcriptional coactivator essential for ligand-dependent transactivation by nuclear receptors in vivo. J. Biol. Chem. 274: 34283-34293, 1999.
Mahajan, M. A.; Samuels, H. H: A new family of nuclear receptor coregulators that integrate nuclear receptor signaling through CREB-binding protein. Molec. Cell. Biol. 20: 5048-5063, 2000.
Zhu, Y.; Kan, L.; Qi, C.; Kanwar, Y. S.; Yeldandi, A. V.; Rao, M. S.; Reddy, J. K.: Isolation and characterization of peroxisome proliferator-activated receptor (PPAR) interacting protein (PRIP) as a coactivator for PPAR. J. Biol. Chem. 275: 13510-13516, 2000
Hirosawa, M.; Nagase, T.; Ishikawa, K.; Kikuno, R.; Nomura, N.; Ohara, O.: Characterization of cDNA clones selected by the GeneMarkanalysis from size-fractionated cDNA libraries from human brain. DNARes. 6: 329-336, 1999.
Battini, J.-L.; Rasko, J. E. J.; Miller, A. D.: A human cell-surface receptor for xenotropic and polytropic murine leukemia viruses: possible role in G protein-coupled signal transduction. Proc. Nat. Acad. Sci. 96:1385-1390, 1999.
Levy, J. A.: Xenotropism: the elusive viral receptor finally uncovered. Proc. Nat. Acad. Sci. 96: 802-804, 1999.
Tailor, C. S.; Nouri, A.; Lee, C. G.; Kozak, C.; Kabat, D.: Cloning and characterization of a cell surface receptor for xenotropic and polytropic murine leukemia viruses. Proc. Nat. Acad. Sci. 96: 927-932, 1999.
Yang, Y.-L.; Guo, L.; Xu, S.; Holland, C. A.; Kitamura, T.; Hunter, K.; Cunningham, J. M.: Receptors for polytropic and xenotropic mouse leukaemia viruses encoded by a single gene at Rmc1. Nature Genet. 21:216-219, 1999.
Hart, M. J.; Callow, M. G.; Souza, B.; Polakis, P.: IQGAP1, a calmodulin-binding protein with a rasGAP-related domain, is a potential effector for cdc42Hs. EMBO J. 15: 2997-3005, 1996
Sugimoto, N.; Imoto, I.; Fukuda, Y.; Kurihara, N.; Kuroda, S.; Tanigami, A.; Kaibuchi, K.; Kamiyama, R.; Inazawa, J.: IQGAP1, a negative regulator of cell-cell adhesion, is up-regulated by gene amplification at 15q26 in gastric cancer cell lines HSC39 and 40A. J. Hum. Genet. 46:21-25, 2001.
Weissbach, L.; Settleman, J.; Kalady, M. F.; Snijders, A. J.; Murthy, A. E.; Yan, Y.-X.; Bernards, A.: Identification of a human Ras GAP-related protein containing calmodulin-binding motifs. J. Biol. Chem. 269:20517-20521, 1994.
Ashford, M. L. J.; Bond, C. T.; Blair, T. A.; Adelman, J. P.: Cloning and functional expression of a rat heart KATP channel. Nature 370:456-459, 1994.
Bond, C. T.; Pessia, M.; Xia, X.-M.; Lagrutta, A.; Kavanaugh, M. P.; Adelman, J. P.: Cloning and expression of a family of inward rectifier potassium channels. Receptors Channels 2: 183-191, 1994.
Tucker, S. J.; James, M. R.; Adelman, J. P.: Assignment of K(ATP)-1, the cardiac ATP-sensitive potassium channel gene (KCNJ5), to human chromosome 11q24. Genomics 28: 127-128, 1995.
Wickman, K.; Seldin, M. F.; Gendler, S. J.; Clapham, D. E.: Partial structure, chromosome localization, and expression of the mouse Girk4 gene. Genomics 40: 395-401, 1997
Jenkins, N. A.: Personal Communication. Frederick, Md. May 12, 1997.
Zhang, Y.; Sun, Z.-W.; Iratni, R.; Erdjument-Bromage, H.; Tempst, P.; Hampsey, M.; Reinberg, D.: SAP30, a novel protein conserved between human and yeast, is a component of a histone deacetylase complex. Molec. Cell 1: 1021-1031, 1998.
Radice, P.; Pensotti, V.; Jones, C.; Perry, H.; Pierotti, M. A.; Tunnacliffe, A.: The human archain gene, ARCN1, has highly conserved homologs in rice and Drosophila. Genomics 26: 101-106, 1995.
Tunnacliffe, A.; van de Vrugt, H.; Pensotti, V.; Radice, P.: The coatomer protein delta-COP, encoded by the archain gene, is conserved across diverse eukaryotes. Mammalian Genome 7: 784-786, 1996.
Langer, L. O., Jr.; Cervenka, J.; Camargo, M.: A severe autosomal recessive acromesomelic dysplasia, the Hunter-Thompson type, and comparison with the Grebe type. Hum. Genet. 81: 323-328, 1989.
Robin, N. H.: Personal Communication. Cleveland, Ohio Nov. 3, 1997.
Runner, M. N.: Linkage of brachypodism: a new member of linkage group V of the house mouse. J. Hered. 50: 81-84, 1959.
Thomas, J. T.; Lin, K.; Nandedkar, M.; Camargo, M.; Cervenka, J.; Luyten, F. P.: A human chondrodysplasia due to a mutation in a TGF-beta super family member. Nature Genet. 12: 315-317, 1996.
Tsumaki, N.; Tanaka, K.; Arikawa-Hirasawa, E.; Nakase, T.; Kimura, T.; Thomas, J. T.; Ochi, T.; Luyten, F. P.; Yamada, Y.: Role of CDMP-lin skeletal morphogenesis: promotion of mesenchymal cell recruitment and chondrocyte differentiation. J. Cell Biol. 144: 161-173, 1999.
Huh, T.-L.; Kim, Y.-O.; Oh, I.-U.; Song, B. J.; Inazawa, J.: Assignment of the human mitochondrial NAD(+)-specific isocitrate dehydrogenase alpha subunit (IDH3A) gene to 15q25.1-q25.2 by in situ hybridization. Genomics 31:295-296, 1996.
Kim, Y.-O.; Oh, I.-U.; Park, H.-S.; Jeng, J.; Song, B. J.; Huh, T.-L.: Characterization of a cDNA clone for human NAD(+)-specific isocitrate dehydrogenase alpha-subunit and structural comparison with its isoenzymes from different species. Biochem. J. 308: 63-68, 1995.
Arinami, T.; Kondo, I.; Hamaguchi, H.; Nakajima, S.: Multi-focal meningiomas in a patient with a constitutional ring chromosome 22. J. Med. Genet. 23: 178-180, 1986
Petrella, R.; Levine, S.; Wilmot, P. L.; Ashar, K. D.; Casamassima, A. C.; Shapiro, L. R.: Multiple meningiomas in a patient with constitutional ring chromosome 22. Am. J. Med. Genet. 47: 184-186, 1993.
Peyrard, M.; Fransson, I.; Xie, Y.-G.; Han, F.-Y.; Ruttledge, M. H.; Swahn, S.; Collins, J. E.; Dunham, I.; Collins, V. P.; Dumanski, J. P.: Characterization of a new member of the human beta-adaptin gene family from chromosome 22q12, a candidate meningioma gene. Hum. Molec. Genet. 3: 1393-1399, 1994.
Peyrard, M.; Pan, H.-Q.; Kedra, D.; Fransson, I.; Swahn, S.; Hartman, K.; Clifton, S. W.; Roe, B. A.; Dumanski, J. P.: Structure of the promoter and genomic organization of the human beta-prime-adaptin gene (BAM22) from chromosome 22q12. Genomics 36: 112-117, 1996.
Zankl, H.; Zang, K. D.: Correlations between clinical and cytogenetical data in 180 human meningiomas. Cancer Genet. Cytogenet. 1: 351-356, 1980.
Harvey, K. F.; Dinudom, A.; Cook, D. I.; Kumar, S.: The Nedd_{—}4-like protein KIAA0439 is a potential regulator of the epithelial sodium channel. J. Biol. Chem. 276: 8597-8601, 2001.
Lees, J. A.; Saito, M.; Vidal, M.; Valentine, M.; Look, T.; Harlow, E.; Dyson, N.; Helin, K.: The retinoblastoma protein binds to a family of E2F transcription factors. Molec. Cell. Biol. 13: 7813-7825, 1993.
Cloud, J. E.; Rogers, C.; Reza, T. L.; Ziebold, U.; Stone, J. R.; Picard, M. H.; Caron, A. M.; Bronson, R. T.; Lees, J. A.: Mutant mouse models reveal the relative roles of E2F1 and E2F3 in vivo. Molec. Cell. Biol. 22: 2663-2672, 2002.
He, Y.; Armanious, M. K.; Thomas, M. J.; Cress, W. D.: Identification of E2F-3B, an alternative form of E2F-3 lacking a conserved N-terminal region. Oncogene 19: 3422-3433, 2000.
Halford, M. M.; Armes, J.; Buchert, M.; Meskenaite, V.; Grail, D.; Hibbs, M. L.; Wilks, A. F.; Farlie, P. G.; Newgreen, D. F.; Hovens, C. M.; Stacker, S. A.: Ryk-deficient mice exhibit craniofacial defects associated with perturbed Eph receptor crosstalk. Nature Genet. 25:414-418, 2000.
Nakamura, S.; Stock, D. W.; Wydner, K. L.; Bollekens, J. A.; Takeshita, K.; Nagai, B. M.; Chiba, S.; Kitamura, T.; Freeland, T. M.; Zhao, Z.; Minowada, J.; Lawrence, J. B.; Weiss, K. M.; Ruddle, F. H.: Genomic analysis of a new mammalian distal-less gene: Dlx7. Genomics 38:314-324, 1996.
Fuhlbrigge, R. C.; Kieffer, J. D.; Armerding, D.; Kupper, T. S.: Cutaneous lymphocyte antigen is a specialized form of PSGL-1 expressed on skin-homing T cells. Nature 389: 978-981, 1997.
Herron, M. J.; Nelson, C. M.; Larson, J.; Snapp, K. R.; Kansas, G. S.; Goodman, J. L.: Intracellular parasitism by the human granulocytic ehrlichiosis bacterium through the P-selectin ligand, PSGL-1. Science 288:1653-1656, 2000.
Veldman, G. M.; Bean, K. M.; Cumming, D. A.; Eddy, R. L.; Sait, S. N. J.; Shows, T. B.: Genomic organization and chromosomal localization of the gene encoding human P-selectin glyco protein ligand. J. Biol. Chem. 270: 16470-16475, 1995.
Yang, J.; Galipeau, J.; Kozak, C. A.; Furie, B. C.; Furie, B.: Mouse P-selectin glyco protein ligand-1: molecular cloning, chromosomal localization, and expression of a functional P-selectin receptor. Blood 87:4176-4186, 1996.
Schwientek, T.; Nomoto, M.; Levery, S. B.; et al: Control of O-glycan branch formation. J. Biol. Chem. 274: 4504-4512, 1999.
Walczak, H.; Degli-Esposti, M. A.; Johnson, R. S.; Smolak, P. J.; Waugh, J. Y.; Boiani, N.; Timour, M. S.; Gerhart, M. J.; Schooley, K. A.; Smith, C. A.; Goodwin, R. G.; Rauch, C. T.: TRAIL-R2: a novel apoptosis-mediating receptor for TRAIL. EMBO J. 16: 5386-5397, 1997.
Wu, G. S.; Burns, T. F.; McDonald, E. R., III; Jiang, W.; Meng, R.; Krantz, I. D.; Kao, G.; Gan, D.-D.; Zhou, J.-Y.; Muschel, R.; Hamilton, S. R.; Spinner, N. B.; Markowitz, S.; Wu, G.; El-Deiry, W. S.: KILLER/DR5 is a DNA damage-inducible p53-regulated death receptor gene. Nature Genet. 17: 141-143, 1997.
Tucker, J. E.; Winkfein, R. J.; Cooper, C. B.; Schnetkamp, P. P.: cDNA cloning of the human retinal rod Na—Ca+K exchanger: comparison with a revised bovine sequence. Invest. Ophthal. Vis. Sci. 39: 435-440, 1998.
Tucker, J. E.; Winkfein, R. J.; Murthy, S. K.; Friedman, J. S.; Walter, M. A.; Demetrick, D. J.; Schnetkamp, P. P. M.: Chromosomal localization and genomic organization of the human retinal rod Na—Ca+K exchanger. Hum. Genet. 103: 411-414, 1998.
Hara, Y.; Wakamori, M.; Ishii, M.; Maeno, E.; Nishida, M.; Yoshida, T.; Yamada, H.; Shimizu, S.; Mori, E.; Kudoh, J.; Shimizu, S.; Kurose, H.; Okada, Y.; Imoto, K.; Mori, Y.: LTRPC2 Ca(2+)-permeable channel activated by changes in redox status confers susceptibility to cell death. Molec. Cell 9: 163-173, 2002.
Harteneck, C.; Plant, T. D.; Schultz, G.: From worm to man: three subfamilies of TRP channels. Trends Neurosci. 23: 159-166, 2000.
Kudoh, J.; Nagamine, K.; Asakawa, S.; Abe, I.; Kawasaki, K.; Maeda, H.; Tsujimoto, S.; Minoshima, S.; Ito, F.; Shimizu, N.: Localization of 16 exons to a 450-kb region involved in the autoimmune polyglandular disease type I (APECED) on human chromosome 21q22.3. DNA Res. 4:45-52, 1997.
Nagamine, K.; Kudoh, J.; Minoshima, S.; Kawasaki, K.; Asakawa, S.; Ito, F.; Shimizu, N.: Molecular cloning of a novel putative Ca(2+)channel protein (TRPC7) highly expressed in brain. Genomics 54:124-131, 1998.
Perraud, A.-L.; Fleig, A.; Dunn, C. A.; Bagley, L. A.; Launay, P.; Schmitz, C.; Stokes, A. J.; Zhu, Q.; Bessman, M. J.; Penner, R.; Kinet, J.-P.; Scharenberg, A. M.: ADP-ribose gating of the calcium-permeable LTRPC2 channel revealed by Nudix motif homology. Nature 411: 595-599, 2001.
Sano, Y.; Inamura, K.; Miyake, A.; Mochizuki, S.; Yokoi, H.; Matsushime, H.; Furuichi, K.: Immunocyte Ca(2+) influx system mediated by LTRPC2. Science 293:1327-1330, 2001.
Cerutti, A.; Schaffer, A.; Goodwin, R. G.; Shah, S.; Zan, H.; Ely, S.; Casali, P.: Engagement of CD153 (CD30 ligand) by CD30-positive T cells inhibits class switch DNA recombination and antibody production in human IgD-positive IgM-positive B cells. J. Immun. 165: 786-794, 2000.
Croager, E. J.; Abraham, L. J.: Characterisation of the human CD30 ligand gene structure. Biochim. Biophys. Acta 1353: 231-235, 1997.
Hurskainen, T. L.; Hirohata, S.; Seldin, M. F.; Apte, S. S.: ADAM-TS5, ADAM-TS6, and ADAM-TS7, novel members of a new family of zinc metalloproteases: general features and genomic distribution of the ADAM-TS family. J. Biol. Chem. 274: 25555-25563, 1999.
Smith, C. A.; Gruss, H.-J.; Davis, T.; Anderson, D.; Farrah, T.; Baker, E.; Sutherland, G. R.; Brannan, C. I.; Copeland, N. G.; Jenkins, N. A.; Grabstein, K. H.; Gliniak, B.; and 9 others: CD30 antigen, a marker for Hodgkin's lymphoma, is a receptor whose ligand defines an emerging family of cytokines with homology to TNF. Cell 73: 1349-1360, 1993.
Gundelfinger, E.: Personal Communication. Madgeburg, FRG. Jan. 8, 1999.
Hashida, H.; Goto, J.; Zhao, N.; Takahashi, N.; Hirai, M.; Kanazawa, I.; Sakaki, Y.: Cloning and mapping of ZNF231, a novel brain-specific gene encoding neuronal double zinc finger protein whose expression is enhanced in a neurodegenerative disorder, multiple system atrophy (MSA). Genomics 54: 50-58, 1998.
Santorelli, F. M.; Patrono, C.; Fortini, D.; Tessa, A.; Comanducci, G.; Bertini, E.; Pierallini, A.; Amabile, G. A.; Casali, C.: Intrafamilial variability in hereditary spastic paraplegia associated with an SPG4 gene mutation. Neurology 55: 702-705, 2000.
Sauter, S.; Miterski, B.; Klimpe, S.; Bonsch, D.; Schols, L.; Visbeck, A.; Papke, T.; Hopf, H. C.; Engel, W.; Deufel, T.; Epplen, J. T.; Neesen, J.: Mutation analysis of the spastin gene (SPG4) inpatients in Germany with autosomal dominant hereditary spastic paraplegia. Hum. Mutat. 20: 127-132, 2002.
Svenson, I. K.; Ashley-Koch, A. E.; Pericak-Vance, M. A.; Marchuk, D. A.: A second leaky splice-site mutation in the spastin gene. (Letter) Am. J. Hum. Genet. 69: 1407-1409, 2001.
Gee, S.; Krauss, S. W.; Miller, E.; Aoyagi, K.; Arenas, J.; Conboy, J. G.: Cloning of mDEAH9, a putative RNA helicase and mammalian homologue of Saccharomyces cerevisiae splicing factor Prp43. Proc. Nat. Acad. Sci. 94: 11803-11807, 1997.
Imamura, O.; Sugawara, M.; Furuichi, Y.: Cloning and characterization of a putative human RNA helicase gene of the DEAH-box protein family. Biochem. Biophys. Res. Commun. 240: 335-340, 1997.
Dominguez, O.; Ashhab, Y.; Sabater, L.; Belloso, E.; Caro, P.; Pujol-Borrell, R.: Cloning of ARE-containing genes by AU-motif-directed display. Genomics 54: 278-286, 1998.
Kostrub, C. F.; Knudsen, K.; Subramani, S.; Enoch, T.: Hus1p, a conserved fission yeast checkpoint protein, interacts with Rad1p and is phosphorylated in response to DNA damage. EMBO J. 17: 2055-2066, 1998.
Katashima, R.; Iwahana, H.; Fujimura, M.; Yamaoka, T.; Ishizuka, T.; Tatibana, M.; Itakura, M.: Molecular cloning of a human cDNA for the 41-kDa phosphoribosylpyrophosphate synthetase-associated protein. Biochim. Biophys. Acta 1396: 245-250, 1998.
Katashima, R.; Iwahana, H.; Fujimura, M.; Yamaoka, T.; Itakura, M.: Assignment of the human phosphoribosylpyrophosphate synthetase-associated protein 41 gene (PRPSAP2) to 17p11.2-p12. Genomics 54: 180-181, 1998.
tom Dieck, S.; Sanmarti-Vila, L.; Langnaese, K.; Richter, K.; Kindler, S.; Soyke, A.; Wex, H.; Smalla, K.-H.; Kampf, U.; Franzer, J.-T.; Stumm, M.; Garner, C. C.; Gundelfinger, E. D.: Bassoon, a novel zinc-finger CAG/glutamine-repeat protein selectively localized at the active zone of presynaptic nerve terminals. J. Cell Biol. 142: 499-509, 1998.
Winter, c.; tom Dieck, S.; Boeckers, T. M.; Bockmann, J.; Kampf, U.; Sanmarti-Vila, L.; Langnaese, K.; Altrock, W.; Stumm, M.; Soyke, A.; Wieacker, P.; Garner, C. C.; Gundelfinger, E. D.: The presynaptic cytomatrix protein Bassoon: sequence and chromosomal localization of the human BSN gene. Genomics 57: 389-397, 1999.
Li, J.; Ding, S.-F.; Habib, N. A.; Fermor, B. F.; Wood, C. B.; Gilmour, R. S.: Partial characterization of a cDNA for human stearoyl-CoA desaturase and changes in its mRNA expression in some normal and malignant tissues. Int. J. Cancer 57: 348-352, 1994.
Ntambi, J. M.; Miyazaki, M.; Stoehr, J. P.; Lan, H.; Kendziorski, C. M.; Yandell, B. S.; Song, Y.; Cohen, P.; Friedman, J. M.; Attie, A. D.: Loss of stearoyl-CoA desaturase-1 function protects mice against adiposity. Proc. Nat. Acad. Sci. 99: 11482-11486, 2002.
Thiede, M. A.; Ozols, J.; Strittmatter, P.: Construction and sequence of cDNA for rat liver stearyl coenzyme A desaturase. J. Biol. Chem. 261:13230-13235, 1986.
Zhang, L.; Ge, L.; Parimoo, S.; Sterm, K.; Prouty, S. M.: Human stearoyl-CoA desaturase: alternative transcripts generated from a single gene by usage of tandem polyadenylation sites. Biochem. J. 340:255-264, 1999.
Zheng, Y.; Eilertsen, K. J.; Ge, L.; Zhang, L.; Sundberg, J. P.; Prouty, S. M.; Sterm, K. S.; Parimoo, S.: Scd1 is expressed in sebaceous glands and is disrupted in the asebia mouse. (Letter) Nature Genet. 23:268-270, 1999.
Kahn, M. L.; Zheng, Y.-W.; Huang, W.; Bigornia, V.; Zeng, D.; Moff, S.; Farese, R. V., Jr.; Tam, C.; Coughlin, S. R.: A dual thrombin receptor system for platelet activation. Nature 394: 690-694, 1998.
Xu, W.-F.; Andersen, H.; Whitmore, T. E.; Presnell, S. R.; Yee, D. P.; Ching, A.; Gilbert, T.; Davie, E. W.; Foster, D. C.: Cloning and characterization of human protease-activated receptor 4. Proc. Nat. Acad. Sci. 95: 6642-6646, 1998.
Matikainen, T.; Perez, G. I.; Jurisicova, A.; Pru, J. K.; Schlezinger, J. J.; Ryu, H.-Y.; Laine, J.; Sakai, T.; Korsmeyer, S. J.; Casper, R. F.; Sherr, D. H.; Tilly, J. L.: Aromatic hydrocarbon receptor-driven Bax gene expression is required for premature ovarian failure caused by biohazardous environmental chemicals. Nature Genet. 28: 355-360, 2001.
Santamarina-Fojo, S.; Peterson, K.; Knapper, C.; Qiu, Y.; Freeman, L.; Cheng, J.-F.; Osorio, J.; Remaley, A.; Yang, X.-P.; Haudenschild, C.; Prades, C.; Chimini, G.; Blackmon, E.; Francois, T.; Duverger, N.; Rubin, E. M.; Rosier, M.; Denefle, P.; Fredrickson, D. S.; Brewer, H. B., Jr.: Complete genomic sequence of the human ABCA1 gene: analysis of the human and mouse ATP-binding cassette A promoter. Proc. Nat. Acad. Sci. 97: 7987-7992, 2000. Note: Erratum: Proc. Nat. Acad. Sci. 99: 1098 only, 2002.
Szakacs, G.; Langmann, T.; Ozvegy, C.; Orso, E.; Schmitz, G.; Varadi, A.; Sarkadi, B.: Characterization of the ATPase cycle of human ABCA1: implications for its function as a regulator rather than an active transporter. Biochem. Biophys. Res. Commun. 288: 1258-1264, 2001.
Utech, M.; Hobbel, G.; Rust, S.; Reinecke, H.; Assmann, G.; Walter, M.: Accumulation of RhoA, RhoB, RhoG, and Rac1 in fibroblasts from Tangier disease subjects suggests a regulatory role of Rho family proteins in cholesterol efflux. Biochem. Biophys. Res. Commun. 280:229-236, 2001.
Zhao, L.-X.; Zhou, C.-J.; Tanaka, A.; Nakata, M.; Hirabayashi, T.; Amachi, T.; Shioda, S.; Ueda, K.; Inagaki, N.: Cloning, characterization and tissue distribution of the rat ATP-binding cassette (ABC) transporter ABC2/ABCA2. Biochem J. 350: 865-872, 2000.
Zwarts, K. Y.; Clee, S. M.; Zwinderman, A. H.; Engert, J. C.; Singaraja, R.; Loubser, O.; James, E.; Roomp, K.; Hudson, T. J.; Jukema, J. W.; Kastelein, J. J. P.; Hayden, M. R.: ABCA1 regulatory variants influence coronary artery disease independent of effects on plasma lipid levels. Clin. Genet. 61: 115-125, 2002.
Kozu, T.; Henrich, B.; Schafer, K. P.: Structure and expression of the gene (HNRPA2B1) encoding the human hnRNP protein A2/B1. Genomics 25:365-371, 1995.
Aruga, J.; Yokota, N.; Hashimoto, M.; Furuichi, T.; Fukuda, M.; Mikoshiba, K.: A novel zinc finger protein, Zic, is involved in neurogenesis, especially in the cell lineage of cerebellar granule cells. J. Neurochem. 63:1880-1890, 1994.
Salero, E.; Perez-Sen, R.; Aruga, J.; Gimenez, C.; Zafra, F.: Transcription factors Zic1 and Zic2 bind and transactivate the apolipoprotein E gene promoter. J. Biol. Chem. 276: 1881-1888, 2001.
Yokota, N.; Aruga, J.; Takai, S.; Yamada, K.; Hamazaki, M.; Iwase, T.; Sugimura, H.; Mikoshiba, K.: Predominant expression of human Zic in cerebellar granule cell lineage and medulloblastoma. Cancer Res. 56: 377-383, 1996.
Tanizawa, Y.; Riggs, A. C.; Dagogo-Jack, S.; Vaxillaire, M.; Froguel, P.; Liu, L.; Donis-Keller, H.; Permutt, M. A.: Isolation of the human LIM/homeo domain gene islet-1 and identification of a simple sequence repeat 1. Diabetes 43: 935-941, 1994.
Shimomura, H.; Sanke, T.; Hanabusa, T.; Tsunoda, K.; Furuta, H.; Nanjo, K.: Nonsense mutation of islet-1 gene (Q310X) found in a type2 diabetic patient with a strong family history. Diabetes 49: 1597-1600, 2000.
Corti, O.; Finocchiaro, G.; Rossi, E.; Zuffardi, O.; DiDonato, S.: Molecular cloning of cDNAs encoding human carnitine acetyltransferase and mapping of the corresponding gene to chromosome 9q34.1. Genomics 23:94-99, 1994.
Kalaria, R. N.; Harik, S. I.: Carnitine acetyltransferase activity in the human brain and its microvessels is decreased in Alzheimer's disease. Ann. Neurol. 32: 583-586, 1992.
van der Leij, F. R.; Huijkman, N. C. A.; Boomsma, C.; Kuipers, J. R. G.; Bartelds, B.: Genomics of the human carnitine acyltransferase genes. Molec. Genet. Metab. 71: 139-153, 2000.
Ghosh, A.: Learning more about NMDA receptor regulation. Science 295:449-451, 2002.
Grunwald, I. C.; Korte, M.; Wolfer, D.; Wilkinson, G. A.; Unsicker, K.; Lipp, H.-P.; Bonhoeffer, T.; Klein, R.: Kinase-independent requirement of EphB2 receptors in hippocampal synaptic plasticity. Neuron 32:1027-1040, 2001
Henderson, J. T.; Georgiou, J.; Jia, Z.; Robertson, J.; Elowe, S.; Roder, J. C.; Pawson, T.: The receptor tyrosine kinase EphB2 regulates NMDA-dependent synaptic function. Neuron 32: 1041-1056, 2001.
Himanen, J.-P.; Rajashankar, K. R.; Lackmann, M.; Cowan, C. A.; Henkemeyer, M.; Nikolov, D. B.: Crystal structure of an Eph receptor-ephrin complex. Nature 414: 933-938, 2001.
Takasu, M. A.; Dalva, M. B.; Zigmond, R. E.; Greenberg, M. E.: Modulation of NMDA receptor-dependent calcium influx and gene expression through EphB receptors. Science 295: 491-495, 2002.
Carlberg, C.; Hooft van Huijsduijnen, R.; Staple, J. K.; DeLamarter, J. F.; Becker-Andre, M.: RZRs, a new family of retinoid-related orphan receptors that function as both monomers and homodimers. Molec. Endocr. 8:757-770, 1994.
Ueda, H. R.; Chen, W.; Adachi, A.; Wakamatsu, H.; Hayashi, S.; Takasugi, T.; Nagano, M.; Nakahama, K.; Suzuki, Y.; Sugano, S.; Iino, M.; Shigeyoshi, Y.; Hashimoto, S.: A transcription factor response element for gene expression during circadian night. Nature 418:534-539, 2002.
Masternak, K.; Barras, E.; Zufferey, M.; Conrad, B.; Corthals, G.; Aebersold, R.; Sanchez, J.-C.; Hochstrasser, D. F.; Mach, B.; Reith, W.: A gene encoding a novel RFX-associated transactivator is mutated in the majority of MHC class II deficiency patients. Nature Genet. 20: 273-277, 1998.
Prange, C. K.; Pennacchio, L. A.; Lieuallen, K.; Fan, W.; Lennon, G. G.: Characterization of the human neurocan gene, CSPG3. Gene 221:199-205, 1998.
Rauch, U.; Grimpe, B.; Kulbe, G.; Arnold-Ammer, I.; Beier, D. R.; Fassler, R.: Structure and chromosomal localization of the mouse neurocan gene. Genomics 28: 405-410, 1995.
Rauch, U.; Karthikeyan, L.; Maurel, P.; Margolis, R. U.; Margolis, R. K.: Cloning and primary structure of neurocan, a developmentally regulated, aggregating chondroitin sulfate proteoglycan of brain. J. Biol. Chem. 267: 19536-19547, 1992.
Barnard, R. C.; Pascall, J. C.; Brown, K. D.; McKay, I. A.; Williams, N. S.; Bustin, S. A.: Coding sequence of ERF-1, the human homologue of Tis11b/cMG1, members of the Tis11 family of early response genes. Nucleic Acids Res. 21: 3580 only, 1993.
Bustin, S. A.; Xiao-Feng, N.; Barnard, R. C.; Kumar, V.; Pascall, J. C.; Brown, K. D.; Leigh, I. M.; Williams, N. S.; McKay, I. A.: Cloning and characterisation of ERF1, a human member of the Tis11 family of early-response genes. DNA Cell Biol. 13: 449-459, 1994.
Maclean, K. N.; See, C. G.; McKay, I. A.; Bustin, S. A.: The human immediate early gene BRF1 maps to chromosome 14q22-q24. Genomics 30:89-90, 1995.
Ning, Z.-Q.; Norton, J. D.; Li, J.; Murphy, J. J.: Distinct mechanisms for rescue from apoptosis in Ramos human B cells by signaling through CD40 and interleukin-4 receptor: a role for inhibition of an early response gene, Berg36. Europ. J. Immun. 26: 2356-2363, 1996.
Maas, S.; Kim, Y.-G.; Rich, A.: Genomic clustering of tRNA-specific adenosine deaminase ADAT1 and two tRNA synthetases. Mammalian Genome 12:387-393, 2001.
Dever, T. E.; Wei, C.-L.; Benkowski, L. A.; Browning, K.; Merrick, W. C.; Hershey, J. W. B.: Determination of the amino acid sequence of rabbit, human, and wheat germ protein synthesis factor eIF-4C byc loning and chemical sequencing. J. Biol. Chem. 269: 3212-3218, 1994.
Adibi, S. A.: The oligopeptide transporter (Pept-1) in human intestine: biology and function. Gastroenterology 113: 332-340, 1997
Fei, Y.-J.; Kanai, Y.; Nussberger, S.; Ganaphthy, V.; Leibach, F. H.; Romero, M. F.; Singh, S. K.; Boron, W. F.; Hediger, M. A.: Expression cloning of a mammalian proton-coupled oligopeptide transporter. Nature 368:563-566, 1994.
Liang, R.; Fei, Y.-J.; Prasad, P. D.; Ramamoorthy, S.; Han, H.; Yang-Feng, T. L.; Hediger, M. A.; Ganaphthy, V.; Leibach, F. H.: Human intestinal H(+)/peptide cotransporter: cloning, functional expression, and chromosomal localization. J. Biol. Chem. 270: 6456-6463, 1995.
Henkemeyer, M.; Orioli, D.; Henderson, J. T.; Saxton, T. M.; Roder, J.; Pawson, T.; Klein, R.: Nuk controls pathfinding of commissural axons in the mammalian central nervous system. Cell 86: 35-46, 1996.
Ikegaki, N.; Tang, X. X.; Liu, X.-G.; Biegel, J. A.; Allen, C.; Yoshioka, A.; Sulman, E. P.; Brodeur, G. M.; Pleasure, D. E.: Molecular characterization and chromosomal localization of DRT (EPHT3): a developmentally regulated human protein-tyrosine kinase gene of the EPH family. Hum. Molec. Genet. 4: 2033-2045, 1995.
Saito, T.; Seki, N.; Matsuda, Y.; Kitahara, M.; Murata, M.; Kanda, N.; Nomura, N.; Yamamoto, T.; Hori, T.: Identification of the human ERK gene as a putative receptor tyrosine kinase and its chromosomal localization to 1p36.1: a comparative mapping of human, mouse, and rat chromosomes. Genomics 26: 382-384, 1995.
Wybenga-Groot, L. E.; Baskin, B.; Ong, S. H.; Tong, J.; Pawson, T.; Sicheri, F.: Structural basis for auto inhibition of the EphB2 receptor tyrosine kinase by the unphosphorylated juxtamembrane region. Cell 106:745-757, 2001.
Reif, K.; Ekland, E. H.; Ohl, L.; Nakano, H.; Lipp, M.; Forster, R.; Cyster, J. G.: Balanced responsiveness to chemoattractants from adjacent zones determines B-cell position. Nature 416: 94-99, 2002.
Inoue, I.; Taniuchi, I.; Kitamura, D.; Jenkins, N. A.; Gilbert, D. J.; Copeland, N. G.; Watanabe, T.: Characteristics of the mouse genomic histamine H1 receptor gene. Genomics 36: 178-181, 1996.
Le Coniat, M.; Traiffort, E.; Ruat, M.; Arrang, J.-M.; Berger, R.: Chromosomal localization of the human histamine H1-receptor gene. Hum. Genet. 94: 186-188, 1994.
Ma, R. Z.; Gao, J.; Meeker, N. D.; Fillmore, P. D.; Tung, K. S. K.; Watanabe, T.; Zachary, J. F.; Offner, H.; Blankenhorn, E. P.; Teuscher, C.: Identification of Bphs, an autoimmune disease locus, as histamine receptor H-1. Science 297: 620-623, 2002.
Yamashita, M.; Fukui, H.; Sugama, K.; Horio, Y.; Ito, S.; Mizuguchi, H.; Wada, H.: Expression cloning of a cDNA encoding the bovine histamine H1 receptor. Proc. Nat. Acad. Sci. 88: 11515-11519, 1991.
Bauer, S.; Groh, V.; Wu, J.; Steinle, A.; Phillips, J. H.; Lanier, L. L.; Spies, T.: Activation of NK cells and T cells by NKG2D, a receptor for stress-inducible MICA. Science 285: 727-729, 1999.
Diefenbach, A.; Jensen, E. R.; Jamieson, A. M.; Raulet, D. H.: Rae1 and H60 ligands of the NKG2D receptor stimulate tumour immunity. Nature 413:165-171, 2001.
Girardi, M.; Oppenheim, D. E.; Steele, C. R.; Lewis, J. M.; Glusac, E.; Filler, R.; Hobby, P.; Sutton, B.; Tigelaar, R. E.; Hayday, A. C.: Regulation of cutaneous malignancy by gamma-delta T cells. Science 294:605-609, 2001.
Groh, V.; Rhinehart, R.; Randolph-Habecker, J.; Topp, M. S.; Riddell, S. R.; Spies, T.: Costimulation of CD8-alpha-beta T cells by NKG2Dvia engagement by MIC induced on virus-infected cells. Nature Immun. 2:255-260, 2001.
Groh, V.; Wu, J.; Yee, C.; Spies, T.: Tumour-derived soluble MIC ligands impair expression of NKG2D and T-cell activation. Nature 419:734-738, 2002.
Li, P.; Morris, D. L.; Willcox, B. E.; Steinle, A.; Spies, T.; Strong, R. K.: Complex structure of the activating immuno receptor NKG2D and its MHC class I-like ligand MICA. Nature Immun. 2: 443-451, 2001
Jacquemin, P.; Durviaux, S. M.; Jensen, J.; Godfraind, C.; Gradwohl, G.; Guillemot, F.; Madsen, O. D.; Carmeliet, P.; Dewerchin, M.; Collen, D.; Rousseau, G. G.; Lemaigre, F. P.: Transcription factor hepatocyte nuclear factor 6 regulates pancreatic endocrine cell differentiation and controls expression of the proendocrine gene ngn3. Molec. Cell. Biol. 20: 4445-4454, 2000.
Jacquemin, P.; Lannoy, V. J.; Rousseau, G. G.; Lemaigre, F. P.: OC-2, a novel mammalian member of the ONE CUT class of homeodomain transcription factors whose function in liver partially overlaps with that of hepatocyte nuclear factor-6. J. Biol. Chem. 274: 2665-2671, 1999.
Lemaigre, F. P.; Durviaux, S. M.; Truong, O.; Lannoy, V. J.; Hsuan, J. J.; Rousseau, G. G.: Hepatocyte nuclear factor 6, a transcription factor that contains a novel type of homeo domain and a single cut domain. Proc. Nat. Acad. Sci. 93: 9460-9464, 1996.
Pierreux, C. E.; Stafford, J.; Demonte, D.; Scott, D. K.; Vandenhaute, J.; O'Brien, R. M.; Granner, D. K.; Rousseau, G. G.; Lemaigre, F. P.: Antiglucocorticoid activity of hepatocyte nuclear factor-6. Proc. Nat. Acad. Sci. 96: 8961-8966, 1999.
Samadani, U.; Costa, R. H.: The transcriptional activator hepatocyte nuclear factor 6 regulates liver gene expression. Molec. Cell. Biol. 16:6273-6284, 1996.
Vaisse, C.; Kim, J.; Espinosa, R., III; Le Beau, M. M.; Stoffel, M.: Pancreatic islet expression studies and polymorphic DNA markers in the genes encoding hepatocyte nuclear factor-3-alpha, -3-beta,-4-gamma, and -6. Diabetes 46: 1364-1367, 1997.
Yamada, K.; Nishida, K.; Hibi, M.; Hirano, T.; Matsuda, Y.: Comparative FISH mapping of Gab1 and Gab2 genes in human, mouse and rat. Cytogenet. Cell Genet. 94: 39-42, 2001.
Kobayashi, H.; Hino, M.; Shimodahira, M.; Iwakura, T.; Ishihara, T.; Ikekubo, K.; Ogawa, Y.; Nakao, K.; Kurahachi, H.: Missense mutation of TRPS1 in a family of trichorhino-phalangeal syndrome type III. Am. J. Med. Genet. 107: 26-29, 2002.
Abuladze, N.; Lee, I.; Newman, D.; Hwang, J.; Boorer, K.; Pushkin, A.; Kurtz, I.: Molecular cloning, chromosomal localization, tissue distribution, and functional expression of the human pancreatic sodium bicarbonate cotransporter. J. Biol. Chem. 273: 17689-17695, 1998.
Burnham, C. E.; Amlal, H.; Wang, Z.; Shull, G. E.; Soleimani, M.: Cloning and functional expression of a human kidney Na+: HCO3− cotransporter. J. Biol. Chem. 272: 19111-19114, 1997.
Choi, I.; Romero, M. F.; Khandoudi, N.; Bril, A.; Boron, W. F.: Cloning and characterization of a human electrogenic Na(+)—HCO(3−)cotransporter isoform (hhNBC). Am. J. Physiol. 276: C576-C584, 1999.
Igarashi, T.; Inatomi, J.; Sekine, T.; Cha, S. H.; Kanai, Y.; Kunimi, M.; Tsukamoto, K.; Satoh, H.; Shimadzu, M.; Tozawa, F.; Mori, T.; Shiobara, M.; Seki, G.; Endou, H.: Mutations in SLC4A4 cause permanent isolated proximal renal tubular acidosis with ocular abnormalities. (Letter) Nature Genet. 23: 264-265, 1999.
Romero, M. F.; Boron, W. F.: Electrogenic Na(+)/HCO(3−) cotransporters: cloning and physiology. Annu. Rev. Physiol. 61: 699-723, 1999.
Soleimani, M.; Burnham, C. E.: Physiologic and molecular aspects of the Na(+): HCO(3−) cotransporter in health and disease processes. Kidney Int. 57: 371-384, 2000.
Usui, T.; et al.; et al.: Pflugers Arch. 438: 458-462, 1999.
Wiley, S. R.; Cassiano, L.; Lofton, T.; Davis-Smith, T.; Winkles, J. A.; Lindner, V.; Liu, H.; Daniel, T. O.; Smith, C. A.; Fanslow, W. C.: A novel TNF receptor family member binds TWEAK and is implicated in angiogenesis. Immunity 15: 837-846, 2001.
Stec, I.; Wright, T. J.; van Ommen, G.-J. B.; de Boer, P. A. J.; van Haeringen, A.; Moorman, A. F. M.; Altherr, M. R.; den Dunnen, J. T.: WHSC1, a 90 kb SET domain-containing gene, expressed in early development and homologous to a Drosophila dysmorphy gene maps in the Wolf-Hirschhorn syndrome critical region and is fused to IgH int(4;14) multiple myeloma. Hum. Molec. Genet. 7: 1071-1082, 1998.
Mao, B.; Wu, W.; Li, Y.; Hoppe, D.; Stannek, P.; Glinka, A.; Niehrs, C.: LDL-receptor-related protein 6 is a receptor for Dickkopf proteins. Nature 411:321-325, 2001.
Hsu, D. R.; Economides, A. N.; Wang, X,; Eimon, P. M.; Harland, R. M.: The Xenopus dorsalizing factor gremlin identifies a novel family of secreted proteins that antagonize BMP activities. Molec. Cell 1: 673-683, 1998.
Topol, L. Z.; Modi, W. S.; Koochekpour, S.; Blair, D. G.: DRM-Gremlin (CKTSF1B1) maps to human chromosome 15 and is highly expressed inadult and fetal brain. Cytogenet. Cell Genet. 89: 79-84, 2000.
Poy, F.; Yaffe, M. B.; Sayos, J.; Saxena, K.; Morra, M.; Sumegi, J.; Cantley, L. C.; Terhorst, C.; Eck, M. J.: Crystal structures of the XLP protein SAP reveal a class of SH2 domains with extended, phosphotyrosine-independent sequence recognition. Molec. Cell 4:555-561, 1999.
Provisor, A. J.; Iacuone, J. J.; Chilcote, R. R.; Neiburger, R. G.; Crussi, F. G.; Baehner, R. L.: Acquired agammaglobulinemia after a life-threatening illness with clinical and laboratory features of infectious mononucleosis in three related male children. New Eng. J. Med. 293: 62-65, 1975.
Purtilo, D. T.: Pathogenesis and phenotypes of an X-linked recessive lymphoproliferative syndrome. Lancet II: 882-885, 1976.
Purtilo, D. T.: X-linked lymphoproliferative syndrome: an immunodeficiency disorder with acquired agammaglobulinemia, fatal infectious mononucleosis, or malignant lymphoma. Arch. Path. Lab. Med. 105: 119-121, 1981.
Purtilo, D. T.; Bhawan, J.; Hutt, L. M.; De Nicola, L.; Szymanski, I.; Yang, J. P. S.; Boto, W.; Naier, R.; Thorley-Lawson, D.: Epstein-Barrvirus in the X-linked recessive lymphoproliferative syndrome. Lancet I: 798-801, 1978.
Purtilo, D. T.; Cassel, C. K.; Yang, J. P. S.: Fatal infectious-mononucleosis in familial lymphohistiocytosis. (Letter) New Eng. J. Med. 201: 736 only, 1974.
Purtilo, D. T.; Cassel, C. K.; Yang, J. P. S.; Harper, R.; Stephenson, S. R.; Landing, B. H.; Vewter, G. F.: X-linked recessive progressive combined variable immunodeficiency (Duncan's disease). Lancet I: 935-941, 1975.
Purtilo, D. T.; DeFlorio, D., Jr.; Hutt, L. M.; Bhawan, J.; Yang, J. P. S.; Otto, R. L.; Edwards, W.: Variable phenotypic expression of an X-linked recessive lymphoproliferative syndrome. New Eng. J. Med. 297: 1077-1081, 1977.
Purtilo, D. T.; Grierson, H. L.: Methods of detection of new families with X-linked lymphoproliferative disease. Cancer Genet. Cytogenet. 51: 143-153, 1991.
Purtilo, D. T.; Sakamoto, K.; Barnabei, V.; Seeley, J.; Bechtold, T.; Rogers, G.; Yetz, J.; Harada, S.; the XLP collaborators: Epstein-Barrvirus-induced diseases in boys with the X-linked lymphoproliferative syndrome (XLP): update on studies of the registry. Am. J. Med. 73:49-56, 1982.
Purtilo, D. T.; Yang, J. P. S.; Allegra, S.; DeFlorio, D.; Hutt, L. M.; Soltani, M.; Vawter, G. F.: Hematopathology and pathogenesis of the X-linked recessive lymphoproliferative syndrome. Am. J. Med. 62:225-233, 1977.
Sanger, W. G.; Grierson, H. L.; Skare, J.; Wyandt, H.; Pirruccello, S.; Fordyce, R.; Purtilo, D. T.: Partial Xq25 deletion in a family with the X-linked lymphoproliferative disease (XLP). Cancer Genet. Cytogenet. 47: 163-169, 1990.
Sayos, J.; Wu, C.; Morra, M.; Wang, N.; Zhang, X.; Allen, D.; van Schaik, S.; Notarangelo, L.; Gehat, R.; Roncarolo, M. G.; Oettgen, H.; De Vries, J. E.; Aversall, G.; Terhorst, C.: The X-linked lymphoproliferative-disease gene product SAP regulates signals induced through the coreceptor SLAM. Nature 395: 462-469, 1998.
Scher, I.: The CBA/N mouse strain: an experimental model illustrating the influence of the X-chromosome on immunity. Adv. Immun. 33: 1-71, 1982.
Schuster, V.; Kreth, H. W.: X-linked lymphoproliferative disease. In: Ochs, H. D.; Smith, C. I. E.; Puck, J. M. (eds.): Primary Immunodeficiency Diseases: A Molecular and Genetic Approach. New York: Oxford University Press 1999. Pp. 222-232.
Seemayer, T. A.; Gross, T. G.; Egeler, R. M.; Pirruccello, S. J.; Davis, D. J.; Kelly, C. M.; Okano, M.; Lanyi, A.; Sumegi, J.: X-linked lymphoproliferative disease: twenty-five years after the discovery. Pediat. Res. 38: 471-478, 1995.
Skare, J.; Grierson, H.; Wyandt, H.; Sanger, W.; Milunsky, J.; Purtilo, D.; Sullivan, J.; Milunsky, A.: Genetics of the X-linked lymphoproliferative syndrome. (Abstract) Am. J. Hum. Genet. 45 (suppl.): A161 only, 1989.
Skare, J.; Madan, S.; Glaser, J.; Purtilo, D.; Nitowsky, H.; Pulijaal, V.; Milunsky, A.: First prenatal diagnosis of X-linked lymphoproliferative disease. Am. J. Med. Genet. 44: 79-81, 1992.
Skare, J.; Milunsky, A.; Byron, K.; Sullivan, J.: The mutation causing X-linked lymphoproliferative syndrome lies in Xq26. (Abstract) Am. J. Hum. Genet. 41: A185 only, 1987.
Skare, J.; Wu, B.-L.; Madan, S.; Pulijaal, V.; Purtilo, D.; Haber, D.; Nelson, D.; Sylla, B.; Grierson, H.; Nitowsky, H.; Glaser, J.; Wissink, J.; White, B.; Holden, J.; Housman, D.; Lenoir, G.; Wyandt, H.; Milunsky, A.: Characterization of three overlapping deletions causing X-linked lymphoproliferative disease. Genomics 16: 254-255, 1993.
Skare, J. C.; Grierson, H. L.; Sullivan, J. L.; Nussbaum, R. L.; Purtilo, D. T.; Sylla, B. S.; Lenoir, G. M.; Reilly, D. S.; White, B. N.; Milunsky, A.: Linkage analysis of seven kindreds with the X-linked lymphoproliferative syndrome (XLP) confirms that the XLP locus is near DXS42 and DXS37. Hum. Genet. 82: 354-358, 1989.
Skare, J. C.; Milunsky, A.; Byron, K. S.; Sullivan, J. L.: Mapping the X-linked lymphoproliferative syndrome. Proc. Nat. Acad. Sci. 84:2015-2018, 1987.
Skare, J. C.; Sullivan, J. L.; Milunsky, A.: Mapping the mutation causing the X-linked lymphoproliferative syndrome in relation to restriction fragment length polymorphisms on Xq. Hum. Genet. 82: 349-353, 1989.
Steinherz, R.; Levy, Y.; Litwin, A.; Nitzan, M.; Friedman, E.; Levin, S.: X-linked lymphoproliferative syndrome: a new kindred with variable phenotypic expression. Am. J. Dis. Child. 139: 191-193, 1985.
Maho, A.; Bensimon, A.; Vassart, G.; Parmentier, M.: Mapping of the CCXCR1, CX3CR1, CCBP2 and CCR9 genes to the CCR cluster within the 3p21.3 region of the human genome. Cytogenet. Cell Genet. 87:265-268, 1999.
Roberts, A. I.; Lee, L.; Schwarz, E.; Groh, V.; Spies, T.; Ebert, E. C.; Jabri, B.: Cutting edge: NKG2D receptors induced by IL-15 costimulate CD28-negative effector CTL in the tissue microenvironment. J. Immun. 167: 5527-5530, 2001.
Ware, R. E.; Howard, T. A.; Kamitani, T.; Chang, H.-M.; Yeh, E. T. H.; Seldin, M. F.: Chromosomal assignment of genes involved inglycosylphosphatidylinositol anchor biosynthesis: implications for the pathogenesis of paroxysmal nocturnal hemoglobinuria. Blood 83:3753-3757, 1994.
Watanabe, R.; Inoue, N.; Westfall, B.; Taron, C. H.; Orlean, P.; Takeda, J.; Kinoshita, T.: The first step of glycosylphosphatidylinositol biosynthesis is mediated by a complex of PIG-A, PIG-H, PIG-C and GPI1. EMBO J. 17: 877-885, 1998.
Watanabe, R.; Kinoshita, T.; Masaki, R.; Yamamoto, A.; Takeda, J.; Inoue, N.: PIG-A and PIG-H, which participate in glycosylphosphatidylinositol anchor biosynthesis, form a protein complex in the endoplasmic reticulum. J. Biol. Chem. 271: 26868-26875, 1996.
Bishop, K. M.; Goudreau, G.; O'Leary, D. D. M.: Regulation of area identity in the mammalian neocortex by Emx2 and Pax6. Science 288:344-349, 2000.
Janz, R.; Sudhof, T. C.; Hammer, R. E.; Unni, V.; Siegelbaum, S. A.; Bolshakov, V. Y.: Essential roles in synaptic plasticity forsynaptogyrin I and synaptophysin I. Neuron 24: 687-700, 1999.
McMahon, H. T.; Bolshakov, V. Y.; Janz, R.; Hammer, R. E.; Siegelbaum, S. A.; Sudhof, T. C.: Synaptophysin, a major synaptic vesicle protein, is not essential for neurotransmitter release. Proc. Nat. Acad. Sci. 93:4760-4764, 1996.
Geppert, M.; Khvotchev, M.; Krasnoperov, V.; Goda, Y.; Missler, M.; Hammer, R. E.; Ichtchenko, K.; Petrenko, A. G.; Sudhof, T. C.: Neurexin I-alpha is a major alpha-Ia-trotoxin receptor that cooperates in alpha-latrotoxin action. J. Biol. Chem. 273: 1705-1710, 1998.
Ichtchenko, K.; Hata, Y.; Nguyen, T.; Ullrich, B.; Missler, M.; Moomaw, C.; Sudhof, T. C.: Neuroligin 1: a splice site-specific ligand for beta-neurexins. Cell 81: 435-443, 1995.
Kleiderlein, J. J.; Nisson, P. E.; Jessee, J.; Li, W.-B.; Becker, K. G.; Derby, M. L.; Ross, C. A.; Margolis, R. L.: CCG repeats in cDNAs from human brain. Hum. Genet. 103: 666-673, 1998. Note: Erratum: Hum. Genet. 104: 113 only, 1999.
Margolis, R.: Personal Communication. Baltimore, Md. Mar. 29, 2000.
Missler, M.; Sudhof, T. C.: Neurexins: three genes and 1001 products. Trends Genet. 14: 20-26, 1998.
Ikeshima, H.; Imai, S.; Shimoda, K.; Hata, J.; Takano, T.: Expression of a MADS box gene, MEF2D, in neurons of the mouse central nervous system: implication of its binary function in myogenic and neurogenic cell lineages. Neurosci. Lett. 200: 117-120, 1995.
Shearman, L. P.; Zylka, M. J.; Weaver, D. R.; Kolakowski, L. F. Jr.; Reppert, S. M.: Two period homologs: circadian expression and photic regulation in the suprachiasmatic nuclei. Neuron 19: 1261-1269, 1997.
Toh, K. L.; Jones, C. R.; He, Y.; Eide, E. J.; Hinz, W. A.; Virshup, D. M.; Ptacek, L. J.; Fu, Y.-H.: An hPer2 phosphorylation site mutationin familial advanced sleep phase syndrome. Science 291: 1040-1043, 2001.
Chien, W.; Pei, L.: A novel binding factor facilitates nuclear translocation and transcriptional activation function of the pituitary tumor-transforming gene product. J. Biol. Chem. 275: 19422-19427, 2000.
Yaspo, M.-L.; Aaltonen, J.; Horelli-Kuitunen, N.; Peltonen, L.; Lehrach, H.: Cloning of a novel human putative type 1a integral membrane protein mapping to 21q22.3. Genomics 49: 133-136, 1998.
Yaspo, M.-L.; Gellen, L.; Mott, R.; Korn, B.; Nizetic, D.; Poustka, A. M.; Lehrach, H.: Model for a transcript map of human chromosome 21: isolation of new coding sequences from exon and enriched cDNA libraries. Hum. Molec. Genet. 4: 1291-1304, 1995.
Liao, C.; Wang, X. Y.; Wei, H. Q.; Li, S. Q.; Merghoub, T.; Pandolfi, P. P.; Wolgemuth, D. J.: Altered myelopoiesis and the development of acute myeloid leukemia in transgenic mice overexpressing cyclin A1. Proc. Nat. Acad. Sci. 98: 6853-6858, 2001.
Liu, D.; Matzuk, M. M.; Sung, W. K.; Guo, Q.; Wang, P.; Wolgemuth, D. J.: Cyclin A1 is required for meiosis in the male mouse. Nature Genet. 20: 377-380, 1998
Muller, C.; Yang, R.; Beck-von-Peccoz, L.; Idos, G.; Verbeek, W.; Koeffler, H. P.: Cloning of the cyclin A1 genomic structure and characterization of the promoter region: GC boxes are essential for cell cycle-regulated transcription of the cyclin A1 gene. J. Biol. Chem. 274: 11220-11228, 1999.
Dolganov, G. M.; Maser, R. S.; Novikov, A.; Tosto, L.; Chong, S.; Bressan, D. A.; Petrini, J. H. J.: Human Rad50 is physically associated with human Mre11: identification of a conserved multi protein compleximplicated in recombinational DNA repair. Molec. Cell Biol. 16:4832-4841, 1996.
Hopfner, K.-P.; Craig, L.; Moncalian, G.; Zinkel, R. A.; Usui, T.; Owen, B. A. L.; Karcher, A.; Henderson, B.; Bodmer, J.-L.; McMurray, C. T.; Carney, J. P.; Petrini, J. H. J.; Tainer, J. A.: The Rad50 zinc-hook is a structure joining Mre11 complexes in DNA recombination and repair. Nature 418: 562-566, 2002.
Luo, G.; Yao, M. S.; Bender, C. F.; Mills, M.; Bladl, A. R.; Bradley, A.; Petrini, J. H. J.: Disruption of mRad50 causes embryonic stem cell lethality, abnormal embryonic development, and sensitivity to ionizing radiation. Proc. Nat. Acad. Sci. 96: 7376-7381, 1999.
Trujillo, K. M.; Yuan, S.-S. F.; Lee, E. Y.-H. P.; Sung, P.: Nuclease activities in a complex of human recombination and DNA repair factors Rad50, Mre11, and p95. J. Biol. Chem. 273: 21447-21450, 1998.
Deiss, L. P.; Feinstein, E.; Berissi, H.; Cohen, O.; Kimchi, A.: Identification of a novel serine/threonine kinase and a novel 15-kD protein as potential mediators of the gamma interferon-induced cell death. Genes Dev. 9: 15-30, 1995.
Feinstein, E.; Druck, T.; Kastury, K.; Berissi, H.; Goodart, S. A.; Overhauser, J.; Kimchi, A.; Huebner, K.: Assignment of DAP1 and DAPK: genes that positively mediate programmed cell death triggered by IFN-gamma—to chromosome regions 5p12.2 (sic) and 9q34.1, respectively. Genomics 29:305-307, 1995.
Kim, D.-H.; Iijima, H.; Goto, K.; Sakai, J.; Ishii, H.; Kim, H.-J.; Suzuki, H.; Kondo, H.; Saeki, S.; Yamamoto, T.: Human apolipoprotein E receptor 2: a novel lipoprotein receptor of the low density lipoprotein receptor family predominantly expressed in brain. J. Biol. Chem. 271:8373-8380, 1996.
Kim, D.-H.; Magoori, K.; Inoue, T. R.; Mao, C. C.; Kim, H.-J.; Suzuki, H.; Fujita, T.; Endo, Y.; Saeki, S.; Yamamoto, T. T.: Exon/intron organization, chromosome localization, alternative splicing, and transcription units of the human apolipoprotein E receptor 2 gene. J. Biol. Chem. 272:8498-8504, 1997.
Campuzano, V.; Montermini, L.; Molto, M. D.; Pianese, L.; Cossee, M.; Cavalcanti, F.; Monros, E.; Rodius, F.; Duclos, F.; Monticelli, A.; Zara, F.; Canizares, J.; Koutnikova, H.; Bidichandani, S. I.; Gellera, C.; Brice, A.; Trouillas, P.; De Michele, G.; Filla, A.; De Frutos, R.; Palau, F.; Patel, P. I.; Di Donato, S.; Mandel, J.-L.; Cocozza, S.; Koenig, M.; Pandolfo, M.: Friedreich's ataxia: autosomal recessive disease caused by an intronic GAA triplet repeat expansion. Science 271: 1423-1427, 1996.
Ilyin, G. P.; Rialland, M.; Pigeon, C.; Guguen-Guillouzo, C.: cDNA cloning and expression analysis of new members of the mammalian F-box protein family. Genomics 67: 40-47, 2000.
Andersson, P.; McGuire, J.; Rubio, C.; Gradin, K.; Whitelaw, M. L.; Pettersson, S.; Hanberg, A.; Poellinger, L.: A constitutively active dioxin/aryl hydrocarbon receptor induces stomach tumors. Proc. Nat. Acad. Sci. 99: 9990-9995, 2002.
Ema, M.; Matsushita, N.; Sogawa, K.; Ariyama, T.; Inazawa, J.; Nemoto, T.; Ota, M.; Oshimura, M.; Fujii-Kuriyama, Y.: Human arylhydrocarbon receptor: functional expression and chromosomal assignment to 7p21. J. Biochem. 116: 845-851, 1994.
Le Beau, M. M.; Carver, L. A.; Espinosa, R., III; Schmidt, J. V.; Bradfield, C. A.: Chromosomal localization of the human AHR locusen coding the structural gene for the Ah receptor to 7p21-p15. Cytogenet. Cell Genet. 66: 172-176, 1994.
Micka, J.; Milatovich, A.; Menon, A.; Grabowski, G. A.; Puga, A.; Nebert, D. W.: Human Ah receptor (AHR) gene: localization to 7p15 and suggestive correlation of polymorphism with CYP1A1 inducibility. Pharmacogenetics 7:95-101, 1997.
Shimizu, Y.; Nakatsuru, Y.; Ichinose, M.; Takahashi, Y.; Kume, H.; Mimura, J.; Fujii-Kuriyama, Y.; Ishikawa, T.: Benzo[a]pyrene carcinogenicity is lost in mice lacking the aryl hydrocarbon receptor. Proc. Nat. Acad. Sci. 97: 779-782, 2000.
Breitbart, R. E.; Liang, C.; Smoot, L. B.; Laheru, D. A.; Mahdavi, V.; Nadal-Ginard, B.: A fourth human MEF2 transcription factor, hMEF2D, is an early marker of the myogenic lineage. Development 118: 1095-1106, 1993.
Hobson, G. M.; Krahe, R.; Garcia, E.; Siciliano, M. J.; Funanage, V. L.: Regional chromosomal assignments for four members of the MADS domain transcription enhancer factor 2 (MEF2) gene family to human chromosomes 15q26, 19p12, 5q14, and 1q12-q23. Genomics 29: 704-711, 1995.
Mao, Z.; Bonni, A.; Xia, F.; Nadal-Vicans, M.; Greenberg, M. E.: Neuronal activity-dependent cell survival mediated by transcription factor MEF2. Science 286: 785-790, 1999.
Martin, J. F.; Miano, J. M.; Hustad, C. M.; Copeland, N. G.; Jenkins, N. A.; Olson, E. N.: A Mef2 gene that generates a muscle-specific isoform via alternative mRNA splicing. Molec. Cell. Biol. 14: 1647-1656, 1994.
Molkentin, J. D.; Black, B. L.; Martin, J. F.; Olson, E. N.: Cooperative activation of muscle gene expression by MEF2 and myogenic bHLH proteins. Cell 83:1125-1136, 1995.
Naya, F. J.; Black, B. L.; Wu, H.; Bassel-Duby, R.; Richardson, J. A.; Hill, J. A.; Olson, E. N.: Mitochondrial deficiency and cardiac sudden death in mice lacking the MEF2A transcription factor. Nature Med. 15 Oct., 2002. Note: Advance Electronic Publication.
Pollock, R.; Treisman, R.: Human SRF-related proteins: DNA-binding properties and potential regulatory targets. Genes Dev. 5: 2327-2341, 1991.
Suzuki, E.; Lowry, J.; Sonoda, G.; Testa, J. R.; Walsh, K.: Structures and chromosome locations of the human MEF2A gene and a pseudogene MEF2AP. Cytogenet. Cell Genet. 73: 244-249, 1996.
Yu, Y.-T.; Breitbart, R. E.; Smoot, L. B.; Lee, Y.; Mahdavi, V.; Nadal-Ginard, B.: Human myocyte-specific enhancer factor 2 comprises a group of tissue-restricted MADS box transcription factors. Genes Dev. 6: 1783-1798, 1992.
Krainc, D.; Haas, M.; Ward, D. C.; Lipton, S. A.; Bruns, G.; Leifer, D.: Assignment of human myocyte-specific enhancer binding factor 2C (hMEF2C) to human chromosome 5q14 and evidence that MEF2C is evolutionarily conserved. Genomics 29: 809-811, 1995.
Leifer, D.; Krainc, D.; Yu, Y.-T.; McDermott, J.; Breitbart, R. E.; Heng, J.; Neve, R. L.; Kosofsky, B.; Nadal-Ginard, B.; Lipton, S. A.: MEF2C, a MADS/MEF2-family transcription factor expressed in a laminar distribution in cerebral cortex. Proc. Nat. Acad. Sci. 90:1546-1550, 1993.
Boie, Y.; Rushmore, T. H.; Darmon-Goodwin, A.; Grygorczyk, R.; Slipetz, D. M.; Metters, K. M.; Abramovitz, M.: Cloning and expression of a cDNA for the human prostanoid IP receptor. J. Biol. Chem. 269:12173-12178, 1994.
Ishikawa, T.; Tamai, Y.; Rochelle, J. M.; Hirata, M.; Namba, T.; Sugimoto, Y.; Ichikawa, A.; Narumiya, S.; Taketo, M. M.; Seldin, M. F.: Mapping of the genes encoding mouse prostaglandin D, E, and F and prostacyclin receptors. Genomics 32: 285-288, 1996.
Murata, T.; Ushikubi, F.; Matsuoka, T.; Hirata, M.; Yamasaki, A.; Sugimoto, Y.; Ichikawa, A.; Aze, Y.; Tanaka, T.; Yoshida, N.; Ueno, A.; Oh-ishi, S.; Narumiya, S.: Altered pain perception and inflammatory response in mice lacking prostacyclin receptor. Nature 388: 678-682, 1997.
Ogawa, Y.; Tanaka, I.; Inoue, M.; Yoshitake, Y.; Isse, N.; Nakagawa, O.; Usui, T.; Itoh, H.; Yoshimasa, T.; Narumiya, S.; Nakao, K.: Structural organization and chromosomal assignment of the human prostacyclin receptor gene. Genomics 27: 142-148, 1995.
Hoffman, I.; Balling, R.: Chromosomal localization of the murine cadherin-11. Mammalian Genome 6: 304 only, 1995.
Okazaki, M.; Takeshita, S.; Kawai, S.; Kikuno, R.; Tsujimura, A.; Kudo, A.; Amann, E.: Molecular cloning and characterization of OB-cadherin, a new member of cadherin family expressed in osteoblasts. J. Biol. Chem. 269: 12092-12098, 1994.
Tanihara, H.; Sano, K.; Heimark, R. L.; St. John, T.; Suzuki, S.: Cloning of five human cadherins clarifies characteristic features of cadherin extracellular domain and provides further evidence for two structurally different types of cadherin. Cell Adhes. Commun. 2:15-26, 1994.
Flanagan, J. R.; Becker, K. G.; Ennist, D. L.; Gleason, S. L.; Driggers, P. H.; Levi, B.-Z.; Appella, E.; Ozato, K.: Cloning of a negative transcription factor that binds to the upstream conserved region of Moloney murine leukemia virus. Molec. Cell. Biol. 12:38-44, 1992.
Hariharan, N.; Kelley, D. E.; Perry, R. P.: Delta, a transcription factor that binds to downstream elements in several polymerase II promoters, is a functionally versatile zinc finger protein. Proc. Nat. Acad. Sci. 88: 9799-9803, 1991.
Oei, S. L.; Shi, Y.: Transcription factor Yin Yang 1 stimulates poly(ADP-ribosyl)ation and DNA repair. Biochem. Biophys. Res. Commun. 284:450-454, 2001.
Park, K.; Atchison, M. L.: Isolation of a candidate repressor/activator, NF-E1 (YY-1, delta), that binds to the immunoglobulin kappa 3-prime enhancer and the immunoglobulin heavy-chain micro-E1 site. Proc. Nat. Acad. Sci. 88: 9804-9808, 1991.
Shi, Y.; Seto, E.; Chang, L.-S.; Shenk, T.: Transcriptional repression by YY1, a human GLI-Kruppel-related protein, and relief of repression by adenovirus E1A protein. Cell 67: 377-388, 1991.
Yao, Y.-L.; Dupont, B. R.; Ghosh, S.; Fang, Y.; Leach, R. J.; Seto, E.: Cloning, chromosomal localization and promoter analysis of the human transcription factor YY1. Nucleic Acids Res. 26: 3776-3783, 1998.
Zhu, W.; Lossie, A. C.; Camper, S. A.; Gumucio, D. L.: Chromosomal localization of the transcription factor YY1 in the mouse and human. Mammalian Genome 5: 234-236, 1994.
Kikuno, R.; Nagase, T.; Ishikawa, K.; Hirosawa, M.; Miyajima, N.; Tanaka, A.; Kotani, H.; Nomura, N.; Ohara, O.: Prediction of the coding sequences of unidentified human genes. XIV. The complete sequences of 100 new cDNA clones from brain which code for large proteins in vitro. DNA Res. 6: 197-205, 1999.
Gao, X.; Copeland, N. G.; Gilbert, D. J.; Jenkins, N. A.; Gridley, T.: Assignment of the murine Notch2 and Notch3 genes to chromosomes 3 and 17. Genomics 49: 160-161, 1998.
Larsson, C.; Lardelli, M.; White, I.; Lendahl, U.: The human NOTCH1,2, and 3 genes are located at chromosome positions 9q34, 1p13-p11, and 19p13.2-p13.1 in regions of neoplasia-associated translocation. Genomics 24:253-258, 1994.
Eudy, J. D.; Yao, S.; Weston, M. D.; Ma-Edmonds, M.; Talmadge, C. B.; Cheng, J. J.; Kimberling, W. J.; Sumegi, J.: Isolation of a gene encoding a novel member of the nuclear receptor super family from the critical region of Usher syndrome type IIa at 1q41. Genomics 50:382-384, 1998.
Greschik, H.; Wurtz, J.-M.; Sanglier, S.; Bourguet, W.; van Dorsselaer, A.; Moras, D.; Renaud, J.-P.: Structural and functional evidence for ligand-independent transcriptional activation by the estrogen-related receptor 3. Molec. Cell 9: 303-313, 2002.
Bertilsson, G.; Heidrich, J.; Svensson, K.; Asman, M.; Jendeberg, L.; Sydow-Backman, M.; Ohlsson, R.; Postlind, H.; Blomquist, P.; Berkenstam, A.: Identification of a human nuclear receptor defines a new signaling pathway for CYP3A induction. Proc. Nat. Acad. Sci. 95: 12208-12213, 1998.
Blumberg, B.; Sabbagh, W., Jr.; Juguilon, H.; Bolado, J., Jr.; van Meter, C. M.; Ong, E. S.; Evans, R. M.: SXR, a novel steroid and xenobiotic-sensing nuclear receptor. Genes Dev. 12: 3195-3205, 1998.
Watkins, R. E.; Wisely, G. B.; Moore, L. B.; Collins, J. L.; Lambert, M. H.; Williams, S. P.; Willson, T. M.; Kliewer, S. A.; Redinbo, M. R.: The human nuclear xenobiotic receptor PXR: structural determinants of directed promiscuity. Science 292: 2329-2333, 2001.
Andre, E.; Conquet, F.; Steinmayr, M.; Stratton, S. C.; Porciatti, V.; Becker-Andre, M.: Disruption of retinoid-related orphan receptor beta changes circadian behavior, causes retinal degeneration and leads to vacillans phenotype in mice. EMBO J. 17: 3867-3877, 1998.
Paravicini, G.; Steinmayr, M.; Andre, E.; Becker-Andre, M.: The metastasis suppressor candidate nucleotide diphosphate kinase NM23 specifically interacts with members of the ROR/RZR nuclear orphan receptor sub family. Biochem. Biophys. Res. Commun. 227: 82-87, 1996.
Sirlin, J. L.: Vacillans, a neurological mutant in the house mouse linked with brown. J. Genet. 54: 42-48, 1956.
Fritzler, M. J.; Lung, C.-C.; Hamel, J. C.; Griffith, K. J.; Chan, E. K. L.: Molecular characterization of golgin-245, a novel Golgi complex protein containing a granin signature. J. Biol. Chem. 270:31262-31268, 1995.
Heard, D. J.; Norby, P. L.; Holloway, J.; Vissing, H.: Human ERR-gamma, a third member of the estrogen receptor-related receptor (ERR) sub family of orphan nuclear receptors: tissue-specific isoforms are expressed during development in the adult. Molec. Endocr. 14: 382-392, 2000.
Hong, H.; Yang, L.; Stallcup, M. R.: Hormone-independent transcriptional activation and coactivator binding by novel orphan nuclear receptor ERR3. J. Biol. Chem. 274: 22618-22626, 1999.
Kooy, J.; Toh, B.-H.; Pettitt, J. M.; Erlich, R.; Gleeson, P. A.: Human autoantibodies as reagents to conserved Golgi components: characterization of a peripheral, 230-kDa compartment-specific Golgi protein. J. Biol. Chem. 267: 20255-20263, 1992.
Zheng, B.; Chen, D.; Farquhar, M. G.: MIR16, a putative membrane glycerophosphodiester phosphodiesterase, interacts with RGS16. Proc. Nat. Acad. Sci. 97: 3999-4004, 2000.
Bergelson, J. M.; Cunningham, J. A.; Droguett, G.; Kurt-Jones, E. A.; Krithivas, A.; Hong, J. S.; Horwitz, M. S.; Crowell, R. L.; Finberg, R. W.: Isolation of a common receptor for coxsackie B viruses and adenoviruses 2 and 5. Science 275: 1320-1323, 1997.
Bowles, K. R.; Gibson, J.; Wu, J.; Shaffer, L. G.; Towbin, J. A.; Bowles, N. E.: Genomic organization and chromosomal localization of the human Coxsackie virus B-adenovirus receptor gene. Hum. Genet. 105:354-359, 1999.
Carson, S. D.; Chapman, N. N.; Tracy, S. M.: Purification of the putative coxsackie virus B receptor from HeLa cells. Biochem. Biophys. Res. Commun. 233: 325-328, 1997.
Griffin, L. D.; Kearney, D.; Ni, J.; Jaffe, R.; Fricker, F. J.; Webber, S.; Demmler, G.; Gelb, B. D.; Towbin, J. A.: Analysis of formalin-fixed and frozen myocardial autopsy samples for viral genome in childhood myocarditis and dilated cardiomyopathy with endocardial fibroelastosis using polymerase chain reaction (PCR). Cardiovasc. Path. 4: 3-11, 1995.
Martin, A. B.; Webber, S.; Fricker, F. J.; Jaffe, R.; Demmler, G.; Kearney, D.; Zhang, Y.-H.; Bodurtha, J.; Gelb, B.; Ni, J.; Bricker, J. T.; Towbin, J. A.: Acute myocarditis: rapid diagnosis by PCR in children. Circulation 90: 330-339, 1994.
Pauschinger, M.; Bowles, N. E.; Fuentes-Garcia, F. J.; Pham, V.; Kuhl, U.; Schwimmbeck, P. L.; Schultheiss, H.-P.; Towbin, J. A.: Detection of adenoviral genome in the myocardium of adult patients with idiopathic left ventricular dysfunction. Circulation 99: 1348-1354, 1999.
Tomko, R. P.; Xu, R.; Philipson, L.: HCAR and MCAR: the human and mouse receptors for subgroup C adenoviruses and group B coxsackie viruses. Proc. Nat. Acad. Sci. 94: 3352-3356, 1997.
Zhang, J.; Kuehl, P.; Green, E. D.; Touchman, J. W.; Watkins, P. B.; Daly, A.; Hall, S. D.; Maurel, P.; Relling, M.; Brimer, C.; Yasuda, K.; Wrighton, S. A.; Hancock, M.; Kim, R. B.; Strom, S.; Thummel, K.; Russell, C. G.; Hudson, J. R., Jr.; Schuetz, E. G.; Boguski, M. S.: The human pregnane X receptor: genomic structure and identification and functional characterization of natural allelic variants. Pharmacogenetics 11:555-572, 2001.
Su, L.-K.; Qi, Y.: Characterization of human MAPRE genes and their proteins. Genomics 71: 143-149, 2001.
Wicki, R.; Marenholz, I.; Mischke, D.; Schafer, B. W.; Heizmann, C. W.: Characterization of the human S100A12 (calgranulin C, p6, CAAF1, CGRP) gene, a new member of the S100 gene cluster on chromosome 1q21. Cell Calcium 20: 459-464, 1996.
Brandt, S.; Jentsch, T. J.: ClC-6 and ClC-7 are two novel broadly expressed members of the CLC chloride channel family. FEBS Lett. 377:15-20, 1995.
Eggermont, J.; Buyse, G.; Voets, T.; Tytgat, J.; De Smedt, H.; Droogmans, G.: Alternative splicing of ClC-6 (a member of the ClC chloride-channel family) transcripts generates three truncated isoforms one of which, ClC-6c, is kidney-specific. Biochem. J. 325: 269-276, 1997.
Nagase, T.; Ishikawa, K.; Suyama, M.; Kikuno, R.; Hirosawa, M.; Miyajima, N.; Tanaka, A.; Kotani, H.; Nomura, N.; Ohara, O.: Prediction of the coding sequences of unidentified human genes. XIII. The complete sequences of 100 new cDNA clones from brain which code for large proteins in vitro. DNA Res. 6: 63-70, 1999.
Arico, M.; Imashuku, S.; Clementi, R.; Hibi, S.; Teramura, T.; Danesino, C.; Haber, D. A.; Nichols, K. E.: Hemophagocytic lymphohistiocytosis due to germline mutations in SH2D1A, the X-linked lymphoproliferative disease gene. Blood 97: 1131-1133, 2001.
Arkwright, P. D.; Makin, G.; Will, A. M.; Ayres, M.; Gokhale, D. A.; Fergusson, W. D.; Taylor, G. M.: X linked lymphoproliferative disease in a United Kingdom family. Arch. Dis. Child. 79: 52-55, 1998.
Bar, R. S.; DeLor, C. J.; Clausen, K. P.; Hurtubise, P.; Henle, W.; Hewetson, J. F.: Fatal infectious mononucleosis in a family. New Eng. J. Med. 290: 363-367, 1974.
Benoit, L.; Wang, X.; Pabst, H. F.; Dutz, J.; Tan, R.: Cutting edge: defective NK cell activation in X-linked lymphoproliferative disease. J. Immun. 165: 3549-3553, 2000.
Coffey, A. J.; Brooksbank, R. A.; Brandau, O.; Oohashi, T.; Howell, G. R.; Bye, J. M.; Cahn, A. P.; Durham, J.; Heath, P.; Wray, P.; Pavitt, R.; Wilkinson, J.; and 31 others: Host response to EBV infection in X-linked lymphoproliferative disease results from mutations in an SH2-domain encoding gene. Nature Genet. 20: 129-135, 1998.
Brandau, O.; Schuster, V.; Weiss, M.; Hellebrand, H.; Fink, F. M.; Kreczy, A.; Friedrich, W.; Strahm, B.; Niemeyer, C.; Belohradsky, B. H.; Meindl, A.: Epstein-Barr virus-negative boys with non-Hodgkin lymphoma are mutated in the SH2D1A gene, as are patients with X-linked lymphoproliferative disease (XLP). Hum. Molec. Genet. 8: 2407-2413, 1999.
Czar, M. J.; Kersh, E. N.; Mijares, L. A.; Lanier, G.; Lewis, J.; Yap, G.; Chen, A.; Sher, A.; Duckett, C. S.; Ahmed, R.; Schwartzberg, P. L.: Altered lymphocyte responses and cytokine production in mice deficient in the X-linked lymphoproliferative disease gene SH2D1A/DSHP/SAP. Proc. Nat. Acad. Sci. 98: 7449-7454, 2001.
Dutz, J. P.; Benoit, L.; Wang, X.; Demetrick, D. J.; Junker, A.; de Sa, D.; Tan, R.: Lymphocytic vasculitis in X-linked lymphoproliferative disease. Blood 97: 95-100, 2001.
Grierson, H. L.; Skare, J.; Church, J.; Silberman, T.; Davis, J. R.; Kobrinsky, N.; McGregor, R.; Israels, S.; McCarty, J.; Andrews, L. G.; Blecha, T.; Erdman, S.; Obringer, A.; Scharnhorst, D.; Purtilo, D. T.: Evaluation of families wherein a single male manifests a phenotype of X-linked lymphoproliferative disease (XLP). Am. J. Med. Genet. 47:458-463, 1993.
Hambleton, G.; Cottom, D. G.: Familial lymphoma. Proc. Roy. Soc. Med. 62: 1095 only, 1969.
Hamilton, J. K.; Paquin, L. A.; Sullivan, J. L.; Maurer, H. S.; Cruzi, F. G.; Provisor, A. J.; Steuber, C. P.; Hawkins, E.; Yawn, D.; Cornet, J.; Clausen, K.; Finkelstein, G. Z.; Landing, B.; Grunnet, M.; Purtilo, D. T.: X-linked lymphoproliferative syndrome registry report. J. Pediat. 96: 669-673, 1980.
Harris, A.; Docherty, Z.: X-linked lymphoproliferative disease: a karyotype analysis. Cytogenet. Cell Genet. 47: 92-94, 1988.
Harris, A.; Lenoir, G. M.; Lankester, S. A.: X-linked lymphoproliferative disease: linkage studies using DNA probes. Clin. Genet. 33: 162-168, 1988.
Hayoz, D.; Lenoir, G. M.; Nicole, A.; Pugin, P.; Regamey, C.: X-linked lymphoproliferative syndrome: identification of a large family in Switzerland. Am. J. Med. 84: 529-534, 1988
Klein, G.; Klein, E.: Sinking surveillance's flagship. Nature 395:441-445, 1998.
Levine, P. H.; Kamaraju, L. S.; Connelly, R. R.; Berard, C. W.; Dorfman, R. F.; Magrath, I.; Easton, J. M.: The American Burkitt's Lymphoma Registry: eight years' experience. Cancer 49: 1016-1022, 1982.
Loeffel, S.; Chang, C.-H.; Heyn, R.; Harada, S.; Lipscomb, H.; Sinangil, F.; Volsky, D. J.; McClain, K.; Ochs, H.; Purtilo, D. T.: Necrotizing lymphoid vasculitis in X-linked lymphoproliferative syndrome. Arch. Path. Lab. Med. 109: 546-550, 1985.
Lyon, M. F.; Loutit, J. F.: X-linked factor in acquired immunodeficiency syndrome?. (Letter) Lancet I: 768 only, 1983.
Mulley, J. C.; Turner, A. M.; Gedeon, A. K.; Berdoukas, V. A.; Huang, T. H. M.; Ledbetter, D. H.; Grierson, H.; Purtilo, D. T.: X-linked lymphoproliferative disease: prenatal detection of an unaffected histocompatible male. Clin. Genet. 42: 76-79, 1992.
Dai, K.-S.; Liew, C.-C.: A novel human striated muscle RING zinc-finger protein, SMRZ, interacts with SMT3b via its RING domain. J. Biol. Chem. 276: 23992-23999, 2001.
Zheng, B.; Albrecht, U.; Kaasik, K.; Sage, M.; Lu, W.; Vaishnav, S.; Li, Q.; Sun, Z. S.; Eichele, G.; Bradley, A.; Lee, C. C.: Nonredundantroles of the mPer1 and mPer2 genes in the mammalian circadian clock. Cell 105:683-694, 2001.
Rulten, S.; Thorpe, J.; Kay, J.: Identification of eukaryotic-parvulin homologues: a new sub family of peptidylprolyl cis-trans isomerases. Biochem. Biophys. Res. Commun. 259: 557-562, 1999.
Ichinose, H.; Ohye, T.; Suzuki, T.; Sumi-Ichinose, C.; Nomura, T.; Hagino, Y.; Nagatsu, T.: Molecular cloning of the human Nurri gene: characterization of the human gene and cDNAs. Gene 230: 233-239, 1999.
Law, S. W.; Conneely, O. M.; DeMayo, F. J.; O'Malley, B. W.: Identification of a new brain-specific transcription factor, NURR1. Molec. Endocr. 6:2129-2135, 1992.
Mages, H. W.; Rilke, O.; Bravo, R.; Senger, G.; Kroczek, R. A.: NOT, a human immediate-early response gene closely related to the steroid/thyroid hormone receptor NAK1/TR3. Molec. Endocr. 8: 1583-1591, 1994.
McEvoy, A. N.; Murphy, E. A.; Ponnio, T.; Conneely, O. M.; Bresnihan, B.; FitzGerald, O.; Murphy, E. P.: Activation of nuclear orphan receptor NURR1 transcription by NF-kappaB and cyclic adenosine 5-prime-monophosphate response element-binding protein in rheumatoid arthritis synovial tissue. J. Immun. 168: 2979-2987, 2002.
Okabe, T.; Takayanagi, R.; Imasaki, K.; Haji, M.; Nawata, H.; Watanabe, T.: cDNA cloning of a NGFI-B/nur77-related transcription factor from an apoptotic human T cell line. J. Immun. 154: 3871-3879, 1995.
Torii, T.; Kawarai, T.; Nakamura, S.; Kawakami, H.: Organization of the human orphan nuclear receptor Nurr1 gene. Gene 230: 225-232, 1999.
Xu, P.-Y.; Liang, R.; Jankovic, J.; Hunter, C.; Zeng, Y.-X.; Ashizawa, T.; Lai, D.; Le, W.-D.: Association of homozygous 7048G7049 variant in the intron six of Nurr1 gene with Parkinson's disease. Neurology 58:881-884, 2002.
Kershaw, D. B.; Beck, S. G.; Wharram, B. L.; Wiggins, J. E.; Goyal, M.; Thomas, P. E.; Wiggins, R. C.: Molecular cloning and characterization of human podocalyxin-like protein: orthologous relationship to rabbit PCLP1 and rat podocalyxin. J. Biol. Chem. 272: 15708-15714, 1997.
Kershaw, D. B.; Wiggins, J. E.; Wharram, B. L.; Wiggins, R. C.: Assignment of the human podocalyxin-like protein (PODXL) gene to 7q32-q33. Genomics 45: 239-240, 1997.
Mankodi, A.; Urbinati, C. R.; Yuan, Q.-P.; Moxley, R. T.; Sansone, V.; Krym, M.; Henderson, D.; Schalling, M.; Swanson, M. S.; Thornton, C. A.: Muscleblind localizes to nuclear foci of aberrant RNA in myotonic dystrophy types 1 and 2. Hum. Molec. Genet. 10: 2165-2170, 2001.
Ichikawa, S.; Ozawa, K.; Hirabayashi, Y.: Assignment of a UDP-glucose: ceramideglucosyltransferase gene (Ugcg) to mouse chromosome band 4B3 by in situ hybridization. Cytogenet. Cell Genet. 83: 14-15, 1998.
Ichikawa, S.; Ozawa, K.; Hirabayashi, Y.: Assignment of a UDP-glucose: ceramideglucosyltransferase gene (UGCG) to human chromosome band 9q31 by in situ hybridization. Cytogenet. Cell Genet. 79: 233-234, 1997.
Ichikawa, S.; Sakiyama, H.; Suzuki, G.; Hidari, K. I.-P. J.; Hirabayashi, Y.: Expression cloning of a cDNA for human ceramide glucosyltransferase that catalyzes the first glycosylation step of glycosphingolipid synthesis. Proc. Nat. Acad. Sci. 93: 4638-4643, 1996.
Watanabe, R.; Wu, K.; Paul, P.; Marks, D. L.; Kobayashi, T.; Pittelkow, M. R.; Pagano, R. E.: Up-regulation of glucosylceramide synthase expression and activity during human keratinocyte differentiation. J. Biol. Chem. 273: 9651-9655, 1998.
Strahm, B.; Rittweiler, K.; Duffner, U.; Brandau, O.; Orlowska-Volk, M.; Karajannis, M. A.; zur Stadt, U.; Tiemann, M.; Reiter, A.; Brandis, M.; Meindl, A.; Niemeyer, C. M.: Recurrent B-cell non-Hodgkin's lymphoma in two brothers with X-linked lymphoproliferative disease without evidence for Epstein-Barr virus infection. Brit. J. Haemat. 108:377-382, 2000.
Sylla, B. S.; Murphy, K.; Cahir-McFarland, E.; Lane, W. S.; Mosialos, G.; Kieff, E.: The X-linked lymphoproliferative syndrome gene product SH2D1A associates with p62(dok) (Dokl) and activates NF-kappa-beta. Proc. Nat. Acad. Sci. 97: 7470-7475, 2000.
Sylla, B. S.; Wang, Q.; Hayoz, D.; Lathrop, G. M.; Lenoir, G. M.: Multipoint linkage mapping of the Xq25-q26 region in a family affected by the X-linked lymphoproliferative syndrome. Clin. Genet. 36:359-462, 1989.
Tangye, S. G.; Lazetic, S.; Woollatt, E.; Sutherland, G. R.; Lanier, L. L.; Phillips, J. H.: Cutting edge: human 2B4, an activating NK cell receptor, recruits the protein tyrosine phosphatase SHP-2 and the adaptor signaling protein SAP. J. Immun. 162: 6981-6985, 1999.
Tangye, S. G.; Phillips, J. H.; Lanier, L. L.; Nichols, K. E.: Cutting edge: functional requirement for SAP in 2B4-mediated activation of human natural killer cells as revealed by the X-linked lymphoproliferative syndrome. J. Immun. 165: 2932-2936, 2000.
Thorley-Lawson, D. A.; Schooley, R. T.; Bhan, A. K.; Nadler, L. M.: Epstein-Barr virus superinduces a new human B cell differentiation antigen (B-LAST 1) expressed on transformed lymphoblasts. Cell 30:415-425, 1982.
Vowels, M. R.; Lam-Po-Tang, R.; Berdoukas, V.; Ford, D.; Thierry, D.; Purtilo, D.; Gluckman, E.: Correction of X-linked lymphoproliferative disease by transplantation of cord-blood stem cells. New Eng. J. Med. 329: 1623-1625, 1993.
Williams, L. L.; Rooney, C. M.; Conley, M. E.; Brenner, M. K.; Krance, R. A.; Heslop, H. E.: Correction of Duncan's syndrome by allogeneic bone marrow transplantation. Lancet 342: 587-588, 1993.
Wu, C.; Nguyen, K. B.; Pien, G. C.; Wang, N.; Gullo, C.; Duncan, H.; Sosa, M. R.; Edwards, M. J.; Borrow, P.; Satoskar, A. R.; Sharpe, A. H.; Biron, C. A.; Terhorst, C.: SAP controls T cell responses to virus and terminal differentiation of T(H)₂ cells. Nature Immun. 2:410-414, 2001.
Wyandt, H. E.; Skare, J. C.; Grierson, H. L.; Purtilo, D. T.; Milunsky, A.: Detection of a chromosomal deletion of Xq25 in an affected male with X-linked lymphoproliferative disease. (Abstract) Am. J. Hum. Genet. 45 (suppl.): A108 only, 1989.
Yin, L.; Ferrand, V.; Lavoue, M.-F.; Hayoz, D.; Philippe, N.; Souillet, G.; Seri, M.; Giacchino, R.; Castagnola, E.; Hodgson, S.; Sylla, B. S.; Romeo, G.: SH2D1A mutation analysis for diagnosis of XLP in typical and atypical patients. Hum. Genet. 105: 501-505, 1999.
Yin, L.; Tocco, T.; Pauly, S.; Lenoir, G. M.; Romeo, G.: Absence of SH2D1A point mutation in 62 Burkitts lymphoma cell lines. Am. J. Hum. Genet. 65 (suppl. 1868): A331 only, 1999.
Dinulos, M. B.; Bassi, M. T.; Rugarli, E. I.; Chapman, V.; Ballabio, A.; Disteche, C. M.: A new region of conservation is defined between human and mouse X chromosomes. Genomics 35: 244-247, 1996.
Schiaffino, M. V.; Bassi, M. T.; Rugarli, E. I.; Renieri, A.; Galli, L.; Ballabio, A.: Cloning of a human homologue of the Xenopus laevis APX gene from the ocular albinism type 1 critical region. Hum. Molec. Genet. 4: 373-382, 1995.
Bachner, D.; Sedlacek, Z.; Korn, B.; Hameister, H.; Poustka, A.: Expression patterns of two human genes coding for different rab GDP-dissociation inhibitors (GDIs), extremely conserved proteins involved in cellular transport. Hum. Molec. Genet. 4: 701-708, 1995
Kang, H.-Y.; Yeh, S.; Fujimoto, N.; Chang, C.: Cloning and characterization of human prostate coactivator ARA54, a novel protein that associates with the androgen receptor. J. Biol. Chem. 274: 8570-8576, 1999.
Foresta, C.; Ferlin, A.; Moro, E.: Deletion and expression analysis of AZFa genes on the human Y chromosome revealed a major role for DBY in male infertility. Hum. Molec. Genet. 9: 1161-1169, 2000.
Shen, P.; Wang, F.; Underhill, P. A.; Franco, C.; Yang, W.-H.; Roxas, A.; Sung, R.; Lin, A. A.; Hyman, R. W.; Vollrath, D.; Davis, R. W.; Cavalli-Sforza, L. L.; Oefner, P. J.: Population genetic implications from sequence variation in four Y chromosome genes. Proc. Nat. Acad. Sci. 97: 7354-7359, 2000.
Scott, D.; Addey, C.; Ellis, P; James, E.; Mitchell, M. J.; Saut, N.; Jurcevic, S.; Simpson, E.: Dendritic cells permit identification of genes encoding MHC class II-restricted epitopes of transplantation antigens. Immunity 12: 711-720, 2000.
Wu, G.; Chai, J.; Suber, T. L.; Wu, J.-W.; Du, C.; Wang, X.; Shi, Y.: Structural basis of IAP recognition by Smac/DIABLO. Nature 408:1008-1012, 2000.
Nakamura, H.; Izumoto, Y.; Kambe, H.; Kuroda, T.; Mori, T.; Kawamura, K.; Yamamoto, H.; Kishimoto, T.: Molecular cloning of complementary DNA for a novel human hepatoma-derived growth factor: its homology with high mobility group-1 protein. J. Biol. Chem. 269: 25143-25149, 1994.
Wanschura, S.; Schoenmakers, E. F. P. M.; Huysmans, C.; Bartnitzke, S.; Van de Ven, W. J. M.; Bullerdiek, J.: Mapping of the gene encoding the human hepatoma-derived growth factor (HDGF) with homology to the high-mobility group (HMG)-1 protein to Xq25. Genomics 32: 298-300, 1996.
Ishibashi, T.; Bottaro, D. P.; Michieli, P.; Kelley, C. A.; Aaronson, S. A.: A novel dual specificity phosphatase induced by serum stimulation and heat shock. J. Biol. Chem. 269: 29897-29902, 1994.
Kwak, S. P.; Dixon, J. E.: Multiple dual specificity protein tyrosine phosphatases are expressed and regulated differentially in liver cell lines. J. Biol. Chem. 270: 1156-1160, 1995.
Lin, J.; Arnold, H. B.; Della-Fera, M. A.; Azain, M. J.; Hartzell, D. L.; Baile, C. A.: Myostatin knockout in mice increases myogenesis and decreases adipogenesis. Biochem. Biophys. Res. Commun. 291:701-706, 2002
McPherron, A. C.; Lawler, A. M.; Lee, S.-J.: Regulation of skeletal muscle mass in mice by a new TGF-beta super family member. Nature 387:83-90, 1997.
Meyerhardt, J. A.; Look, A. T.; Bigner, S. H.; Fearon, E. R.: Identification and characterization of neogenin, a DCC-related gene. Oncogene 14:1129-1136, 1997.
Vielmetter, J.; Chen, X.-N.; Miskevich, F.; Lane, R. P.; Yamakawa, K.; Korenberg, J. R.; Dreyer, W. J.: Molecular characterization of human neogenin, a DCC-related protein, and the mapping of its gene (NEO1) to chromosomal position 15q22.3-q23. Genomics 41: 414-421, 1997.
Vielmetter, J.; Kayyem, J. F.; Roman, J. M.; Dreyer, W. J.: Neogenin, an avian cell surface protein expressed during terminal neuronal differentiation, is closely related to the human tumor suppressor molecule deleted in colorectal cancer. J. Cell Biol. 127: 2009-2020, 1994.
Donghi, R.; Sozzi, G.; Pierotti, M. A.; Biunno, I.; Miozzo, M.; Fusco, A.; Grieco, M.; Santoro, M.; Vecchio, G.; Spurr, N. K.; DellaPorta, G.: The oncogene associated with human papillary thyroid carcinoma (PTC) is assigned to chromosome 10 q11-q12 in the same region as multiple endocrine neoplasia type 2A (MEN2A). Oncogene 4: 521-523, 1989
Nikiforova, M. N.; Stringer, J. R.; Blough, R.; Medvedovic, M.; Fagin, J. A.; Nikiforov, Y. E.: Proximity of chromosomal loci that participate in radiation-induced rearrangements in human cells. Science 290:138-141, 2000.
Sozzi, G.; Pierotti, M. A.; Miozzo, M.; Donghi, R.; Radice, P.; De Benedetti, V.; Grieco, M.; Santoro, M.; Fusco, A.; Vecchio, G.; Mathew, C. G. P.; Ponder, B. A. J.; Spurr, N. K.: Refined localization to contiguous regions on chromosome 10q of the two genes (H4 and RET) that form the oncogenic sequence PTC. Oncogene 6: 339-342, 1991.
Tong, Q.; Li, Y.; Smanik, P. A.; Fithian, L. J.; Xing, S.; Mazzaferri, E. L.; Jhiang, S. M.: Characterization of the promoter region and oligomerization domain of H4 (D10S170), a gene frequently rearranged with the ret proto-oncogene. Oncogene 10: 1781-1787, 1995.
Spicer, A. P.; Olson, J. S.; McDonald, J. A.: Molecular cloning and characterization of a cDNA encoding the third putative mammalian hyaluronan synthase. J. Biol. Chem. 272: 8957-8961, 1997.
Bingle, C. D.; Gowan, S.: Molecular cloning of the forkhead transcription factor HNF-3-alpha from a human pulmonary adenocarcinoma cell line. Biochim. Biophys. Acta 1307: 17-20, 1996
Cushman, L. J.; Camper, S. A.: Molecular basis of pituitary dysfunction in mouse and human. Mammalian Genome 12: 485-494, 2001.
Biamonti, G.; Ruggiu, M.; Saccone, S.; Della Valle, G.; Riva, S.: Two homologous genes, originated by duplication, encode the human hnRNP proteins A2 and A1. Nucleic Acids Res. 22: 1996-2002, 1994.
Nagase, T.; Seki, N.; Ishikawa, K.; Ohira, M.; Kawarabayasi, Y.; Ohara, O.; Tanaka, A.; Kotani, H.; Miyajima, N.; Nomura, N.: Prediction of the coding sequences of unidentified human genes. VI. The coding sequences of 80 new genes (KIAA0201-KIAA0280) deduced by analysis of cDNA clones from cell line KG-1 and brain. DNA Res. 3: 321-329, 1996. Note: Supplement: DNA Res. 3: 341-354, 1996.
Numata, M.; Petrecca, K.; Lake, N.; Orlowski, J.: Identification of a mitochondrial Na+/H+ exchanger. J. Biol. Chem. 273: 6951-6959, 1998.
Nakano, H.; Oshima, H.; Chung, W.; Williams-Abbott, L.; Ware, C. F.; Yagita, H.; Okumura, K.: TRAF5, an activator of NF-kappaB and putative signal transducer for the lymphotoxin-beta receptor. J. Biol. Chem. 271: 14661-14664, 1996.
Nakano, H.; Sakon, S.; Koseki, H.; Takemori, T.; Tada, K.; Matsumoto, M.; Munechika, E.; Sakai, T.; Shirasawa, T.; Akiba, H; Kobata, T.; Santee, S. M.; Ware, C. F.; Renner, P. D.; Taniguchi, M.; Yagita, H.; Okumura, K.: Targeted disruption of Traf5 gene causes defects in CD40- and CD27-mediated lymphocyte activation. Proc. Nat. Acad. Sci. 96: 9803-9808, 1999.
Nakano, H.; Shindo, M.; Yamada, K.; Yoshida, M. C.; Santee, S. M.; Ware, C. F.; Jenkins, N. A.; Gilbert, D. J.; Yagita, H.; Copeland, N. G.; Okumura, K.: Human TNF receptor associated factor 5 (TRAF5): cDNA cloning, expression and assignment of the TRAF5 gene to chromosome 1q32. Genomics 42: 26-32, 1997.
Fernandez-Valle, C.; Tang, Y.; Ricard, J.; Rodenas-Ruano, A.; Taylor, A.; Hackler, E.; Biggerstaff, J.; Iacovelli, J.: Paxillin binds schwannomin and regulates its density-dependent localization and effect on cell morphology. Nature Genet. 31: 354-362, 2002.
Glenney, J. R., Jr.; Zokas, L.: Novel tyrosine kinase substrates from Rous sarcoma virus-transformed cells are present in the membrane skeleton. J. Cell Biol. 108: 2401-2408, 1989.
Mazaki, Y.; Hashimoto, S.; Sabe, H.: Monocyte cells and cancer cells express novel paxillin isoforms with different binding properties to focal adhesion proteins. J. Biol. Chem. 272: 7437-7444, 1997.
Salgia, R.; Li, J.-L.; Lo, S. H.; Brunkhorst, B.; Kansas, G. S.; Sobhany, E. S.; Sun, Y.; Pisick, E.; Hallek, M.; Ernst, T.; Tantravahi, R.; Chen, L. B.; Griffin, J. D.: Molecular cloning of human paxillin, a focal adhesion protein phosphorylated by P210(BCR/ABL). J. Biol. Chem. 270: 5039-5047, 1995.
Turner, C. E.; Glenney, J. R., Jr.; Burridge, K.: Paxillin: anew vinculin-binding protein present in focal adhesions. J. Cell Biol. 111: 1059-1068, 1990.
Adachi, H.; Tsujimoto, M.; Hattori, M.; Arai, H.; Inoue, K.: Differential tissue distribution of the beta- and gamma subunits of human cytosolic platelet-activating factor acetylhydrolase (isoform 1). Biochem. Biophys. Res. Commun. 233: 10-13, 1997.
Moro, F.; Arrigo, G.; Fogli, A.; Bernard, L.; Carrozzo, R.: The beta and gamma subunits of the human platelet-activating factor acetylhydrolase isoform 1b (PAFAH1B2 and PAFAH1B3) map to chromosome 11q23 and 19q13.1, respectively. Genomics 51: 157-159, 1998.
Daigo, Y.; Isomura, M.; Nishiwaki, T.; Tamari, M.; Ishikawa, S.; Kai, M.; Murata, Y.; Takeuchi, K.; Yamane, Y.; Hayashi, R.; Minami, M.; Fujino, M. A.; Hojo, Y.; Uchiyama, I.; Takagi, T.; Nakamura, Y.: Characterization of a 1200-kb genomic segment of chromosome 3p22-p21.3. DNA Res. 6: 37-44, 1999.
Erlich, R.; Gleeson, P. A.; Campbell, P.; Dietzsch, E.; Toh, B.-H.: Molecular characterization of trans-Golgi p230: a human peripheral membrane protein encoded by a gene on chromosome 6p12-22 contains extensive coiled-coil alpha-helical domains and a granin motif. J. Biol. Chem. 271: 8328-8337, 1996.
Giachino, C.; Lantelme, E.; Lanzetti, L.; Saccone, S.; Della Valle, G.; Migone, N.: A novel SH3-containing human gene family preferentially expressed in the central nervous system. Genomics 41: 427-434, 1997.
Shisheva, A.; Sudhof, T. C.; Czech, M. P.: Cloning, characterization, and expression of a novel GDP dissociation inhibitor isoform from skeletal muscle. Molec. Cell. Biol. 14: 3459-3468, 1994.
Peters, A. H. F. M.; O'Carroll, D.; Scherthan, H.; Mechtler, K.; Sauer, S.; Schofer, C.; Weipoltshammer, K.; Pagani, M.; Lachner, M.; Kohlmaier, A.; Opravil, S.; Doyle, M.; Sibilia, M.; Jenuwein, T.: Loss of the Suv39h histone methyltransferases impairs mammalian heterochromatin and genome stability. Cell 107: 323-337, 2001
Haltiwanger, R. S.; Blomberg, M. A.; Hart, G. W.: Glycosylation of nuclear and cytoplasmic proteins: purification and characterization of a uridine diphospho-N-acetylglucosamine: polypeptide beta-N-acetylglucosaminyltransferase. J. Biol. Chem. 267: 9005-9013, 1992.
Kreppel, L. K.; Blomberg, M. A.; Hart, G. W.: Dynamic glycosylation of nuclear and cytosolic proteins: cloning and characterization of a unique O-GlcNAc transferase with multiple tetratricopeptide repeats. J. Biol. Chem. 272: 9308-9315, 1997.
Lubas, W. A.; Frank, D. W.; Krause, M.; Hanover, J. A.: O-linked GlcNAc transferase is a conserved nucleocytoplasmic protein containing tetratricopeptide repeats. J. Biol. Chem. 272: 9316-9324, 1997.
Shafi, R.; Iyer, S. P. N.; Ellies, L. G.; O'Donnell, N.; Marek, K. W.; Chui, D.; Hart, G. W.; Marth, J. D.: The O-GlcNAc transferase gene resides on the X chromosome and is essential for embyronic stem cell viability and mouse ontogeny. Proc. Nat. Acad. Sci. 97: 5735-5739, 2000.
Yang, X.; Zhang, F.; Kudlow, J. E.: Recruitment of OGlcNAc transferase to promoters by corepressor mSin3A: coupling protein O-GlcNAcylation to transcriptional repression. Cell 110: 69-80, 2002.
Gecz, J.; Baker, E.; Donnelly, A.; Ming, J. E.; McDonald McGinn, D. M.; Spinner, N. B.; Zackai, E. H.; Sutherland, G. R.; Mulley, J. C.: Fibroblast growth factor homologous factor 2 (FHF2): gene structure, expression and mapping to the Borjeson-Forssman-Lehmann syndrome region in Xq26 delineated by a duplication breakpoint in a BFLS-like patient. Hum. Genet. 104: 56-63, 1999.
Lovec, H.; Hartung, H.; Verdier, A.-S.; Mattei, M.-G.; Birnbaum, D.; Goldfarb, M.; Coulier, F.: Assignment of FGF13 to human chromosome band Xq21 by in situ hybridization. Cytogenet. Cell Genet. 76: 183-184, 1997.
Smallwood, P. M.; Munoz-Sanjuan, I.; Tong, P.; Macke, J. P.; Hendry, S. H. C.; Gilbert, D. J.; Copeland, N. G.; Jenkins, N. A.; Nathans, J.: Fibroblast growth factor (FGF) homologous factors: new members of the FGF family implicated in nervous system development. Proc. Nat. Acad. Sci. 93: 9850-9857, 1996.
Nauseef, W. M.; Brigham, S.; Cogley, M.: Hereditary myeloperoxidase deficiency due to a missense mutation of arginine 569 to tryptophan. J. Biol. Chem. 269: 1212-1216, 1994.
Busard, B. L. S. M.; Renier, W. O.; Gabreels, F. J. M.; Jaspar, H. H. J.; van Haelst, U. J. G.; Slooff, J. L.: Lafora's disease: comparison of inclusion bodies in skin and in brain. Arch. Neurol. 43:296-299, 1986.
Busard, H. L. S. M.; Gabreels-Festen, A. A. W. M.; Renier, W. O.; Gabreels, F. J. M.; Stadhouders, A. M.: Axilla skin biopsy: a reliable test for the diagnosis of Lafora's disease. Ann. Neurol. 21: 599-601, 1987.
Fluharty, A. L.; Porter, M. T.; Hirsh, G. A.; Pevida, E.; Kihara, H.: Metachromasia in fibroblasts from a patient with Lafora's disease. (Letter) Lancet II: 109-110, 1970.
Ganesh, S.; Agarwala, K. L.; Ueda, K.; Akagi, T.; Shoda, K.; Usui, T.; Hashikawa, T.; Osada, H.; Delgado-Escueta, A. V.; Yamakawa, K.: Laforin, defective in the progressive myoclonus epilepsy of Laforatype, is a dual-specificity phosphatase associated with polyribosomes. Hum. Molec. Genet. 9: 2251-2261, 2000.
Gomez-Garre, P.; Sanz, Y.; Rodriguez de Cordoba, S.; Serratosa, J. M.: Mutational spectrum of the EPM2A gene in progressive myoclonus epilepsy of Lafora: high degree of allelic heterogeneity and prevalence of deletions. Europ. J. Hum. Genet. 8: 946-954, 2000.
Harriman, D. G. F.; Millar, J. H. D.: Progressive familial myoclonic epilepsy in 3 families: its clinical features and pathological basis. Brain 78:325-349, 1955.
Janeway, R.; Ravens, J. R.; Pearce, L. A.; Odor, D. L.; Suzuki, K.: Progressive myoclonus epilepsy with Lafora inclusion bodies. I. Clinical, genetic, histopathologic and biochemical aspects. Arch. Neurol. 16: 565-582, 1967.
Lehesjoki, A.-E.; Koskiniemi, M.; Pandolfo, M.; Antonelli, A.; Kyllerman, M.; Wahlstrom, J.; Nergardh, A.; Burmeister, M.; Sistonen, P.; Norio, R.; de la Chapelle, A.: Linkage studies in progressive myoclonus epilepsy: Unverricht-Lundborg and Lafora's diseases. Neurology 42:1545-1550, 1992.
Maddox, L. O.; Descartes, M.; Collins, J.; Keating, J.; Rosenfeld, S.; Palmer, C.; Carroll, A. J.; Kuzniecky, R.: Identification of a recombination event narrowing the Lafora disease gene region. J. Med. Genet. 34: 590-591, 1997.
Solinas-Toldo, S.; Lengauer, C.; Fries, R.: Comparative genome map of human and cattle. Genomics 27: 489-496, 1995.
Olives, B.; Martial, S.; Mattei, M.-G.; Matassi, G.; Rousselet, G.; Ripoche, P.; Cartron, J.-P.; Bailly, P.: Molecular characterization of a new urea transporter in the human kidney. FEBS Lett. 386: 156-160, 1996.
Ranade, K.; Wu, K.-W.; Hwu, C.-M.; Ting, C.-T.; Pei, D.; Pesich, R.; Hebert, J.; Chen, Y.-D. I.; Pratt, R.; Olshen, R.; Masaki, K.; Risch, N.; Cox, D. R.; Botstein, D.: Genetic variation in the human urea transporter-2 is associated with variation in blood pressure. Hum. Molec. Genet. 10: 2157-2164, 2001.
Ansel, K. M.; Ngo, V. N.; Hyman, P. L.; Luther, S. A.; Forster, R.; Sedgwick, J. D.; Browning, J. L.; Lipp, M.; Cyster, J. G.: Achemokine-driven positive feedback loop organizes lymphoid follicles. Nature 406:309-314, 2000.
Petersen, S.; Casellas, R.; Reina-San-Martin, B.; Chen, H. T.; Difilippantonio, M. J.; Wilson, P. C.; Hanitsch, L.; Celeste, A.; Muramatsu, M.; Pil; ch, D. R.; Redon, C.; Ried, T.; Bonner, W. M.; Honjo, T.; Nussenzweig, M. C.; Nussenzweig, A.: AID is required to initiate Nbs1/gamma-H2AX focus formation and mutations at sites of class switching. Nature 414:660-665, 2001.
Morasso, M. I.; Yonescu, R.; Griffin, C. A.; Sargent, T. D.: Localization of human DLX8 to chromosome 17q21.3-q22 by fluorescence in situ hybridization. Mammalian Genome 8: 302-303, 1997.
Quinn, L. M.; Johnson, B. V.; Nicholl, J.; Sutherland, G. R.; Kalionis, B.: Isolation and identification of homeobox genes from the human placenta including a novel member of the Distal-less family, DLX4. Gene 187:55-61, 1997.
Bengtsson, E.; Neame, P. J.; Heinegard, D.; Sommarin, Y.: The primary structure of a basic leucine-rich repeat protein, PRELP, found in connective tissues. J. Biol. Chem. 270: 25639-25644, 1995.
Grover, J.; Chen, X.-N.; Korenberg, J. R.; Recklies, A. D.; Roughley, P. J.: The gene organization, chromosome location, and expression of a 55-kDa matrix protein (PRELP) of human articular cartilage. Genomics 38:109-117, 1996.
Blanco, P.; Sargent, C. A.; Boucher, C. A.; Mitchell, M.; Affara, N. A.: Conservation of PCDHX in mammals; expression of X/Y genes predominantly in brain. Mammalian Genome 11: 906-914, 2000.
Ciccodicola, A.; D'Esposito, M.; Esposito, T.; Gianfrancesco, F.; Migliaccio, C.; Miano, M. G.; Matarazzo, M. R.; Vacca, M.; Franze, A.; Cuccurese, M.; Cocchia, M.; Curci, A.; and 9 others: Differentially regulated and evolved genes in the fully sequenced Xq/Yq pseudoautosomal region. Hum. Molec. Genet. 9: 395-401, 2000.
Mumm, S.; Molini, B.; Terrell, J.; Srivastava, A.; Schlessinger, D.: Evolutionary features of the 4-Mb Xq21.3 XY homology region revealed by a map at 60-kb resolution. Genome Res. 7: 307-314, 1997
Schwartz, A.; Chan, D. C.; Brown, L. G.; Alagappan, R.; Pettay, D.; Disteche, C.; McGillivray, B.; de la Chapelle, A.; Page, D. C.: Reconstructing hominid Y evolution: Xhomologous block, created by X-Y transposition, was disrupted by Yp inversion through LINE-LINE recombination. Hum. Molec. Genet. 7: 1-11, 1998.
Tilford, C. A.; Kuroda-Kawaguchi, T.; Skaletsky, H.; Rozen, S.; Brown, L. G.; Rosenberg, M.; McPherson, J. D.; Wylie, K.; Sekhon, M.; Kucaba, T. A.; Waterston, R. H.; Page, D. C.: A physical map of the human Y chromosome. Nature 409: 943-945, 2001.
Yoshida, K.; Sugano, S.: Identification of a novel protocadherin gene (PCDH11) on the human XY homology region in Xq21.3. Genomics 62:540-543, 1999.
Liu, Z.; Sun, C.; Olejniczak, E. T.; Meadows, R. P.; Betz, S. F.; Oost, T.; Herrmann, J.; Wu, J. C.; Fesik, S. W.: Structural basis for binding of Smac/DIABLO to the XIAP BIR3 domain. Nature 408:1004-1008, 2000.
Srinivasula, S. M.; Hegde, R.; Saleh, A.; Datta, P.; Shiozaki, E.; Chai, J.; Lee, R.-A.; Robbins, P. D.; Fernandes-Alnemri, T.; Shi, Y.; Alnemri, E. S.: A conserved XIAP-interaction motif in caspase-9 and Smac/DIABLO regulates caspase activity and apoptosis. Nature 410:112-116, 2001.
Bomont, P.; Cavalier, L.; Blondeau, F.; Ben Hamida, C.; Belal, S.; Tazir, M.; Demir, E.; Topaloglu, H.; Korinthenberg, R.; Tuysuz, B.; Landrieu, P.; Hentati, F.; Koenig, M.: The gene encoding gigaxonin, a new member of the cytoskeletal BTB/kelch repeat family, is mutatedin giant axonal neuropathy. Nature Genet. 26: 370-374, 2000.
Treiber-Held, S.; Budjarjo-Welim, H.; Riemann, D.; Richter, J.; Kretzschmar, H. A.; Hanefeld, F.: Giant axonal neuropathy: a generalizeddisorder of intermediate filaments with longitudinal grooves in the hair. Neuropediatrics 25: 89-93, 1994.
Minassian, B. A.; Lee, J. R.; Herbrick, J.-A.; Huizenga, J.; Soder, S.; Mungall, A. J.; Dunham, I.; Gardner, R.; Fong, C. G.; Carpenter, S.; Jardim, L.; Satishchandra, P.; Andermann, E.; Snead, O. C., III; Lopes-Cendes, I.; Tsui, L.-C.; Delgado-Escueta, A. V.; Rouleau, G. A.; Scherer, S. W.: Mutations in a gene encoding a novel proteintyrosine phosphatase cause progressive myoclonus epilepsy. Nature Genet. 20: 171-174, 1998.
Norio, R.; Koskiniemi, M.: Progressive myoclonus epilepsy: genetic and nosological aspects with special reference to 107 Finnish patients. Clin. Genet. 15: 382-398, 1979
Ortiz-Hidalgo, C.: The man behind Lafora's bodies. Am. J. Surg. Path. 10: 358-361, 1986.
Sainz, J.; Minassian, B. A.; Serratosa, J. M.; Gee, M. N.; Sakamoto, L. M.; Iranmanesh, R.; Bohlega, S.; Baumann, R. J.; Ryan, S.; Sparkes, R. S.; Delgado-Escueta, A. V.: Lafora progressive myoclonus epilepsy: narrowing the chromosome 6q24 locus by recombinations and homozygosities. (Letter) Am. J. Hum. Genet. 61: 1205-1209, 1997.
Sarlin, M. B.; Kloepfer, H. W.; Mickle, W. A.; Heath, R. G.: The detection of carriers in hereditary myoclonic epilepsy. Acta Genet. Med. Gemellol. 9: 466-471, 1960.
Schwarz, G. A.; Yanoff, M.: Lafora's disease, distinct clinico-pathologic form of Unverricht's syndrome. Arch. Neurol. 12: 172-188, 1965.
Serratosa, J. M.; Delgado-Escueta, A. V.; Posada, I.; Shih, S.; Drury, I.; Berciano, J.; Zabala, J. A.; Antunez, M. C.; Sparkes, R. S.: The gene for progressive myoclonus epilepsy of the Lafora type maps to chromosome 6q. Hum. Molec. Genet. 4: 1657-1663, 1995.
Yanoff, M.; Schwarz, G. A.: Lafora's disease: a distinct genetically determined form of Unverricht's syndrome. Genet. Hum. 14: 235-244, 1965.
Yokoi, S.; Austin, J.; Witmer, F.; Sakai, M.: Studies in myoclonus epilepsy (Lafora body forms). I. Isolation and preliminary characterization of Lafora bodies in two cases. Arch. Neurol. 19: 15-33, 1968.
Serratosa, J. M.; Gomez-Garre, P.; Gallardo, M. E.; Anta, B.; Beltran-Valero de Bernabe, D.; Lindhout, D.; Augustijn, P. B.; Tassinari, C. A.; Michelucci, R.; Malafosse, A.; Topcu, M.; Grid, D.; Dravet, C.; Berkovic, S. F.; Rodriguez de Cordoba, S.: A novel protein tyrosine phosphatase gene is mutated in progressive myoclonus epilepsy of theLafora type (EPM2). Hum. Molec. Genet. 8: 345-352, 1999.
Carney, J. P.; Maser, R. S.; Olivares, H.; Davis, E. M.; Le Beau, M.; Yates, J. R., III; Hays, L.; Morgan, W. F.; Petrini, J. H. J.: The hMre11/hRad50 protein complex and Nijmegen breakage syndrome: linkage of double-strand break repair to the cellular DNA damage response. Cell 93:477-486, 1998.
Chang, S. C.; Hoang, B.; Thomas, J. T.; Vukicevic, S.; Luyten, F. P.; Ryba, N. J. P.; Kozak, C. A.; Reddi, A. H.; Moos, M., Jr.: Cartilage-derived morphogenetic proteins: new members of the transforming growth factor-beta super family predominantly expressed in long bones during human embryonic development. J. Biol. Chem. 269: 28227-28234, 1994
Rousseau-Merck, M.-F.; Duro, D.; Berger, R.; Thiesen, H. J.: Chromosomal localization of two KOX zinc finger genes on chromosome bands 7q21-q22. Ann. Genet. 38: 81-84, 1995.
Tommerup, N.; Vissing, H.: Isolation and fine mapping of 16 novel human zinc finger-encoding cDNAs identify putative candidate genes for developmental and malignant disorders. Genomics 27: 259-264, 1995.
Faiyaz-UI-Haque, M.; Ahmad, W.; Wahab, A.; Haque, S.; Azim, A. C.; Zaidi, S. H. E.; Teebi, A. S.; Ahmad, M.; Cohn, D. H.; Siddique, T.; Tsui, L.-C.: Frameshift mutation in the cartilage-derived morphogenetic protein 1 (CDMP1) gene and severe acromesomelic chondrodysplasia resembling Grebe-type chondrodysplasia. Am. J. Med. Genet. 111: 31-37, 2002.
Boring, L.; Gosling, J.; Cleary, M.; Charo, I. F.: Decreased lesion formation in CCR2 −/− mice reveals a role for chemokines in the initiation of atherosclerosis. Nature 394: 894-897, 1998.
Charo, I. F.; Myers, S. J.; Herman, A.; Franci, C.; Connolly, A. J.; Coughlin, S. R.: Molecular cloning and functional expression of two monocyte chemoattractant protein 1 receptors reveals alternative splicing of the carboxyl-terminal tails. Proc. Nat. Acad. Sci. 91:2752-2756, 1994.
Chang, C.; da Silva, S. L.; Ideta, R.; Lee, Y.; Yeh, S.; Burbach, J. P. H.: Human and rat TR4 orphan receptors specify a subclass of the steroid receptor super family. Proc. Nat. Acad. Sci. 91: 6040-6044, 1994.
Chang, C.; Kokontis, J.; Acakpo-Satchivi, L.; Liao, S.; Takeda, H.; Chang, Y.: Molecular cloning of new human TR2 receptors: a class of steroid receptor with multiple ligand-binding domains. Biochem. Biophys. Res. Commun. 165: 735-741, 1989.
Hirose, T.; Fujimoto, W.; Yamaai, T.; Kim, K. H.; Matsuura, H.; Jetten, A. M.: TAK1: Molecular cloning and characterization of anew member of the nuclear receptor super family. Molec. Endocr. 1667-1680, 1994.
Yoshikawa, T.; DuPont, B. R.; Leach, R. J.; Detera-Wadleigh, S. D.: New variants of the human and rat nuclear hormone receptor, TR4: expression and chromosomal localization of the human gene. Genomics 35:361-366, 1996.
Choe, H.; Farzan, M.; Sun, Y.; Sullivan, N.; Rollins, B.; Ponath, P. D.; Wu, L.; Mackay, C. R.; LaRosa, G.; Newman, W.; Gerard, N.; Gerard, C.; Sodroski, J.: The beta-chemokine receptors CCR3 and CCR5 facilitate infection by primary HIV-1 isolates. Cell 85: 1135-1148, 1996.
Kim, B.-T.; Kitagawa, H.; Tamura, J.; Saito, T.; Kusche-Gullberg, M.; Lindahl, U.; Sugahara, K.: Human tumor suppressor EXT gene family members EXTL1 and EXTL3 encode alpha-1,4-N-acetylglucosaminyl transferases that likely are involved in heparan sulfate/heparin biosynthesis. Proc. Nat. Acad. Sci. 98: 7176-7181, 2001.
Wise, C. A.; Clines, G. A.; Massa, H.; Trask, B. J.; Lovett, M.: Identification and localization of the gene for EXTL, a third member of the multiple exostoses gene family. Genome Res. 7: 10-16, 1997.
Kobayashi, S.; Uemura, H.; Kohda, T.; Nagai, T.; Chinen, Y.; Naritomi, K.; Kinoshita, E.; Ohashi, H.; Imaizumi, K.; Tsukahara, M.; Sugio, Y.; Tonoki, H.; Kishino, T.; Tanaka, T.; Yamada, M.; Tsutsumi, O.; Niikawa, N.; Kaneko-Ishino, T.; Ishino, F.: No evidence of PEG1/MEST gene mutations in Silver-Russell syndrome patients. Am. J. Med. Genet. 104:225-231, 2001.
Bosher, J. M.; Williams, T.; Hurst, H. C.: The developmentally regulated transcription factor AP-2 is involved in cerbB-2 overexpression in human mammary carcinoma. Proc. Nat. Acad. Sci. 92: 744-747, 1995.
Combadiere, C.; Ahuja, S. K.; Murphy, P. M.: Cloning, chromosomal localization, and RNA expression of a human beta chemokine receptor-like gene. DNA Cell Biol. 14: 673-680, 1995.
Combadiere, C.; Ahuja, S. K.; Van Damme, J.; Tiffany, H. L.; Gao, J.-L.; Murphy, P. M.: Monocyte chemoattractant protein-3 is a functional ligand for CC chemokine receptors 1 and 2B. J. Biol. Chem. 270:29671-29675, 1995.
Doranz, B. J.; Rucker, J.; Yi, Y.; Smyth, R. J.; Samson, M.; Peiper, S. C.; Parmentier, M.; Collman, R. G.; Doms, R. W.: A dual-tropic primary HIV-1 isolate that uses fusin and the beta-chemokine CKR-5, CKR-3, and CKR-2b as fusion cofactors. Cell 85: 1149-1158, 1996.
Mummidi, S.; Ahuja, S. S.; Gonzalez, E.; Anderson, S. A.; Santiago, E. N.; Stephan, K. T.; Craig, F. E.; O'Connell, P.; Tryon, V.; Clark, R. A.; Dolan, M. J.; Ahuja, S. K.: Genealogy of the CCR5 locus and chemokine system gene variants associated with altered rates of HIV-1 disease progression. Nature Med. 4: 786-793, 1998.
Peters, W.; Dupuis, M.; Charo, I. F.: A mechanism for the impaired IFN-gamma production in C-C chemokine receptor 2 (CCR2) knockout mice: role of CCR2 in linking the innate and adaptive immune responses. J. Immun. 165: 7072-7077, 2000
Peters, W.; Scott, H. M.; Chambers, H. F.; Flynn J. L.; Charo, I. F.; Ernst, J. D.: Chemokine receptor 2 serves an early and essential role in resistance to Mycobacterium tuberculosis. Proc. Nat. Acad. Sci. 98: 7958-7963, 2001.
Samson, M.; Labbe, O.; Mollereau, C.; Vassart, G.; Parmentier, M.: Molecular cloning and functional expression of a new human CC-chemokine receptor gene. Biochemistry 35: 3362-3367, 1996.
Samson, M.; Soularue, P.; Vassart, G.; Parmentier, M.: The genes encoding the human CC-chemokine receptors CCCKR1 to CC-CKR5 (CMKBR1-CMKBR5) are clustered in the p21.3-p24 region of chromosome 3. Genomics 36:522-526, 1996.
Sanders, S. K.; Crean, S. M.; Boxer, P. A.; Kellner, D.; LaRosa, G. J.; Hunt, S. W., III.: Functional differences between monocyte chemotactic protein-1 receptor A and monocyte chemotactic protein-1 receptor B expressed in a Jurkat T cell. J. Immun. 165: 4877-4883, 2000.
Smith, M. W.; Dean, M.; Carrington, M.; Winkler, C.; Huttley, G. A.; Lomb, D. A.; Goedert, J. J.; O'Brien, T. R.; Jacobson, L. P.; Kaslow, R.; Buchbinder, S.; Vittinghoff, E.; Vlahov, D.; Hoots, K.; Hilgartner, M. W.; Hemophilia Growth and Development Study (HGDS); Multicenter AIDS Cohort Study (MACS); Multicenter Hemophilia Cohort Study (MHCS); San Francisco City Cohort (SFCC); ALIVE Study; O'Brien, S. J.: Contrasting genetic influence of CCR2 and CCR5 variants on HIV-1 infection and disease progression. Science 277: 959-965, 1997.
Wong, L.-M.; Myers, S. J.; Tsou, C.-L.; Gosling, J.; Arai, H.; Charo, I. F.: Organization and differential expression of the human monocyte chemoattractant protein 1 receptor gene: evidence for the role of the carboxyl-terminal tail in receptor trafficking. J. Biol. Chem. 272: 1038-1045, 1997.
Donald, L. J.; Wang, H. S.; Hamerton, J. L.: Assignment of the gene for cystathionase (CYS) to human chromosome 16. (Abstract) Cytogenet. Cell Genet. 32: 268 only, 1982.
Frimpter, G. W.: Cystathioninuria: nature of the defect. Science 149:1095-1096, 1965.
Frimpter, G. W.: Cystathioninuria, sulfite oxidase deficiency, and ‘beta-mercaptolactate-cysteine disulfiduria. In: Stanbury, J. B.; Wyngaarden, J. B.; Fredrickson, D. S.: The Metabolic Basis of Inherited Disease. New York: McGraw-Hill (pub.) (3rd ed.): 1972. Pp. 413-425.
Frimpter, G. W.; Haymovitz, A.; Hor with, M.: Cystathioninuria. New Eng. J. Med. 268: 333-339, 1963.
Harris, H.; Penrose, L. S.; Thomas, D. H. H.: Cystathioninuria. Ann. Hum. Genet. 23: 442-453, 1959.
Lu, Y.; O'Dowd, B. F.; Orrego, H.; Israel, Y.: Cloning and nucleotide sequence of human liver cDNA encoding for cystathionine gamma-lyase. Biochem. Biophys. Res. Commun. 189: 749-758, 1992.
Lyon, I. C. T.; Procopis, P. G.; Turner, B.: Cystathioninuriain a well baby population. Acta Paediat. Scand. 60: 324-328, 1971.
Mongeau, J.-G.; Hilgartner, M.; Worthen, H. G.; Frimpter, G. W.: Cystathioninuria: study of an infant with normal mentality, thrombocytopenia, and renal calculi. J. Pediat. 69: 1113-1120, 1967.
Pascal, T. A.; Gaull, G. E.; Beratis, N. G.; Gillam, B. M.; Tallan, H. H.: Cystathionase deficiency: evidence for genetic heterogeneity in primary cystathioninuria. Pediat. Res. 12: 125-133, 1978.
Perry, T. L.; Hardwick, D. F.; Hansen, S.; Love, D. L.; Israels, S.: Cystathioninuria in two healthy siblings. New Eng. J. Med. 278:590-592, 1968.
Schneiderman, L. J.: Latent cystathioninuria. J. Med. Genet. 4:260-263, 1967.
Scott, C. R.; Dassell, S. W.; Clark, S. H.; Chiang-Teng, C.; Swedberg, K. R.: Cystathioninemia: a benign genetic condition. J. Pediat. 76:571-577, 1970.
Shaw, K. N. F.; Lieberman, E.; Koch, R.; Donnell, G. N.: Cystathioninuria. Am. J. Dis. Child. 113: 119-128, 1967.
Tada, K.; Yoshida, T.; Yokoyama, Y.; Sato, T.; Nakagawa, H.; Arakawa, T.: Cystathioninuria not associated with vitamin B6 dependency: a probably new type of cystathioninuria. Tohoku J. Exp. Med. 95: 235-242, 1968.
Whelan, D. T.; Scriver, C. R.: Cystathioninuria and renal iminoglycinuria in a pedigree: a perspective on counseling. New Eng. J. Med. 278:924-927, 1968.
Nagase, T.; Seki, N.; Ishikawa, K.; Ohira, M.; Kawarabayasi, Y.; Ohara, O.; Tanaka, A.; Kotani, H.; Miyajima, N.; Nomura, N.: Prediction of the coding sequences of unidentified human genes. VI. The coding sequences of 80 new genes (KIAA0201-KIAA0280) deduced by analysis of cDNA clones from cell line KG-1 and brain. DNA Res. 3: 321-329, 1996.
Connelly, M. A.; Grady, R. C.; Mushinski, J. F.; Marcu, K. B.: PANG, a gene encoding a neuronal glyco protein, is ectopically activated by intracisternal A-type particle long terminal repeats in murine plasmacytomas. Proc. Nat. Acad. Sci. 91: 1337-1341, 1994.
Mock, B. A.; Connelly, M. A.; McBride, O. W.; Kozak, C. A.; Marcu, K. B.: Plasmacytoma-associated neuronal glyco protein, Pang, maps to mouse chromosome 6 and human chromosome 3. Genomics 34: 226-228, 1996.
Cheung, A. H.; Stewart, R. J.; Marsden, P. A.: Endothelial Tie2/Tek ligands angiopoietin-1 (ANGPT1) and angiopoietin-2 (ANGPT2): regional localization of the human genes to 8q22.3-q23 and 8p23. Genomics 48:389-391, 1998.
Davis, S.; Aldrich, T. H.; Jones, P. F.; Acheson, A.; Compton, D. L.; Jain, V.; Ryan, T. E.; Bruno, J.; Radziejewski, C.; Maisonpierre, P. C.; Yancopoulos, G. D.: Isolation of angiopoietin-1, a ligand for the TIE2 receptor, by secretion-trap expression cloning. Cell 87:1161-1169, 1996.
Folkman, J.; D'Amore, P. A.: Blood vessel formation: what is its molecular basis? Cell 87: 1153-1155, 1996.
Marziliano, N.; Crovella, S.; Audero, E.; Pecile, V.; Bussolino, F.; Amoroso, A.; Garagna, S.: Genetic mapping of the mouse homologue of the human angiopoietin-1 gene (Agpt) to mouse chromosome 9E2 byin situ hybridization. Cytogenet. Cell Genet. 87: 199-200, 1999.
Sato, T. N.; Tozawa, Y.; Deutsch, U.; Wolburg-Buchholz, K.; Fujiwara, Y.; Gendron-Maguire, M.; Gridley, T.; Wolburg, H.; Risau, W.; Qin, Y.: Distinct roles of the receptor tyrosine kinases Tie-1 and Tie-2 in blood vessel formation. Nature 376: 70-73, 1995
Suri, C.; Jones, P. F.; Patan, S.; Bartunkova, S.; Maisonpierre, P. C.; Davis, S.; Sato, T. N.; Yancopoulos, G. D.: Requisite role of angiopoietin-1, a ligand for the TIE2 receptor, during embryonic angiogenesis. Cell 87: 1171-1180, 1996.
Suri, C.; McClain, J.; Thurston, G.; McDonald, D. M.; Zhou, H.; Oldmixon, E. H.; Sato, T. N.; Yancopoulos, G. D.: Increased vascularization in mice overexpressing angiopoietin-1. Science 282: 468-471, 1998.
Valenzuela, D. M.; Griffiths, J. A.; Rojas, J.; Aldrich, T. H.; Jones, P. F.; Zhou, H.; McClain, J.; Copeland, N. G.; Gilbert, D. J.; Jenkins, N. A.; Huang, T.; Papadopoulos, N.; Maisonpierre, P. C.; Davis, S.; Yancopoulos, G. D.: Angiopoietins 3 and 4: diverging gene counterparts in mice and humans. Proc. Nat. Acad. Sci. 96:1904-1909, 1999.
Nakanishi-Matsui, M.; Zheng, Y.-W.; Sulciner, D. J.; Weiss, E. J.; Ludeman, M. J.; Coughlin, S. R.: PAR3 is a cofactor for PAR4 activation by thrombin. Nature 404: 609-613, 2000.
Sambrano, G. R.; Weiss, E. J.; Zheng, Y.-W.; Huang, W.; Coughlin, S. R.: Role of thrombin signalling in platelets in haemostasis and thrombosis. Nature 413: 74-78, 2001.
Faiyaz-UI-Haque, M.; Ahmad, W.; Zaidi, S. H. E.; Haque, S.; Teebi, A. S.; Ahmad, M.; Cohn, D. H.; Tsui, L. C.: Mutation in the cartilage-derived morphogenetic protein-1 (CDMP1) gene in a kindred affected with fibular hypoplasia and complex brachydactyly (DuPan syndrome). Clin. Genet. 61:454-458, 2002.
Shapiro, M. B.; Senaphthy, P.: RNA splice junctions of different classes of eukaryotes: sequence statistics and functional implications in gene expression. Nucleic Acids Res. 15: 7155-7174, 1987.
Ishikawa, K.; Nagase, T.; Suyama, M.; Miyajima, N.; Tanaka, A.; Kotani, H.; Nomura, N.; Ohara, O.: Prediction of the coding sequences of unidentified human genes. X. The complete sequences of 100 new cDNA clones from brain which can code for large proteins in vitro. DNA Res. 5: 169-176, 1998.
Kipreos, E. T.; Lander, L. E.; Wing, J. P.; He, W. W.; Hedgecock, E. M.: cul-1 is required for cell cycle exit in C. elegans and identifies a novel gene family. Cell 85: 829-839, 1996.
Daugherty, B. L.; Springer, M. S.: The beta-chemokine receptor genes CCR1 (CMKBR1), CCR2 (CMKBR2), and CCR3 (CMKBR3) cluster within 285 kb on human chromosome 3p21. Genomics 41: 294-295, 1997.
Ferbus, D.; Le Chalony, C.; Prosperi, M.-T.; Muleris, M.; Vincent-Salomon, A.; Goubin, G.: Identification, nuclear localization, and binding activities of OZF, a human protein solely composed of zinc finger motifs. Europ. J. Biochem. 236: 991-995, 1996.
Le Chalony, C.; Apiou, F.; Pibouin, L.; Dutrillaux, B.; Goubin, G.: Constitutive amplification of a zinc finger protein gene in cattle. DNA Cell Biol. 15: 83-88, 1996.
Le Chalony, C.; Prosperi, M.-T.; Haluza, R.; Apiou, F.; Dutrillaux, B.; Goubin, G.: The OZF gene encodes a protein consisting essentially of zinc-finger motifs. J. Molec. Biol. 236: 399-404, 1994.
Dhar, S. K.; Yoshida, K.; Machida, Y.; Khaira, P.; Chaudhuri, B.; Wohlschlegel, J. A.; Leffak, M.; Yates, J.; Dutta, A.: Replication from oriP of Epstein-Barr virus requires human ORC and is inhibited by geminin. Cell 106: 287-296, 2001.
Gavin, K. A.; Hidaka, M.; Stillman, B.: Conserved initiator proteins in eukaryotes. Science 270: 1667-1671, 1995.
Ohtani, K.; DeGregori, J.; Leone, G.; Herendeen, D. R.; Kelly, T. J.; Nevins, J. R.: Expression of the HsOrc1 gene, a human ORC1 homolog, is regulated by cell proliferation via the E2F transcription factor. Molec. Cell. Biol. 16: 6977-6984, 1996.
Takahara, K.; Bong, M.; Brevard, R.; Eddy, R. L.; Haley, L. L.; Sait, S. J.; Shows, T. B.; Hoffman, G. G.; Greenspan, D. S.: Mouse and human homologues of the yeast origin of replication recognition complex subunit ORC2 and chromosomal localization of the cognate human gene ORC2L. Genomics 31: 119-122, 1996.
Rowen, L.; Young, J.; Birditt, B.; Kaur, A.; Madan, A.; Philipps, D. L.; Qin, S.; Minx, P.; Wilson, R. K.; Hood, L.; Graveley, B. R.: Analysis of the human neurexin genes: alternative splicing and the generation of protein diversity. Genomics 79: 587-597, 2002.
Tabuchi, K.; Sudhof, T. C.: Structure and evolution of neurexin genes: insight into the mechanism of alternative splicing. Genomics 79:849-859, 2002.
Ullrich, B.; Ushkaryov, Y. A.; Sudhof, T. C.: Cartography of neurexins: more than 1000 isoforms generated by alternative splicing and expressed in distinct subsets of neurons. Neuron 14: 497-507, 1995.
Ushkaryov, Y. A.; Petrenko, A. G.; Geppert, M.; Sudhof, T. C.: Neurexins: synaptic cell surface proteins related to the alpha-latrotoxin receptor and laminin. Science 257: 50-56, 1992.
Scheiffele, P.; Fan, J.; Choih, J.; Fetter, R.; Serafini, T.: Neuroligin expressed in nonneuronal cells triggers presynaptic development in contacting axons. Cell 101: 657-669, 2000.
Cavaloc, Y.; Popielarz, M.; Fuchs, J.-P.; Gattoni, R.; Stevenin, J.: Characterization and cloning of the human splicing factor 9G8: a novel 35 kDa factor of the serine/arginine protein family. EMBO J. 13: 2639-2649, 1994.
Popielarz, M.; Cavaloc, Y.; Mattei, M.-G.; Gattoni, R.; Stevenin, J.: The gene encoding human splicing factor 9G8: structure, chromosomal localization, and expression of alternatively processed transcripts. J. Biol. Chem. 270: 17830-17835, 1995.
Wu, Q.; Zhang, T.; Cheng, J.-F.; Kim, Y.; Grimwood, J.; Schmutz, J.; Dickson, M.; Noonan, J. P.; Zhang, M. Q.; Myers, R. M.; Maniatis, T.: Comparative DNA sequence analysis of mouse and human protocadherin gene clusters. Genome Res. 11: 389-404, 2001.
Aoki, K.; Inazawa, J.; Takahashi, T.; Nakahara, K.; Kasai, M.: Genomic structure and chromosomal localization of the gene encoding translin, a recombination hotspot binding protein. Genomics 43:237-241, 1997.
Aoki, K.; Nakahara, K.; Ikegawa, C.; Seto, M.; Takahashi, T.; Minowada, J.; Strominger, J. L.; Maziarz, R. T.; Kasai, M.: Nuclear proteins binding to a novel target sequence within the recombination hotspot regions of Bcl-2 and the immunoglobulin D(H) gene family. Oncogene 9:1109-1115, 1994.
Bodzioch, M.; Orso, E.; Klucken, J.; Langmann, T.; Bottcher, A.; Diederich, W.; Drobnik, W.; Barlage, S.; Buchler, C.; Porsch-Ozcurumez, M.; Kaminski, W. E.; Hahmann, H. W.; Oette, K.; Rothe, G.; Aslanidis, C.; Lackner, K. J.; Schmitz, G.: The gene encoding ATP-binding cassette-transporter 1 is mutated in Tangier disease. Nature Genet. 22: 347-351, 1999.
Brooks-Wilson, A.; Marcil, M.; Clee, S. M.; Zhang, L.-H.; Roomp, K.; van Dam, M.; Yu, L.; Brewer, C.; Collins, J. A.; Molhuizen, H. O. F.; Loubser, O.; Ouelette, B. F. F.; and 14 others: Mutations in ABC1 in Tangier disease and familial high-density lipoprotein deficiency. Nature Genet. 22: 336-345, 1999.
Remaley, A. T.; Rust, S.; Rosier, M.; Knapper, C.; Naudin, L.; Broccardo, C.; Peterson, K. M.; Koch, C.; Arnould, I.; Prades, C.; Duverger, N.; Funke, H.; Assman, G.; Dinger, M.; Dean, M.; Chimini, G.; Santamarina-Fojo, S.; Fredrickson, D. S.; Denefle, P. Brewer, H. B., Jr.: Human ATP-binding cassette transporter 1 (ABC1): genomic organization and identification of the genetic defect in the original Tangier disease kindred. Proc. Nat. Acad. Sci. 96: 12685-12690, 1999.
Rust, S.; Rosier, M.; Funke, H.; Real, J.; Amoura, Z.; Piette, J.-C.; Deleuze, J.-F.; Brewer, H. B.; Duverger, N.; Denefle, P.; Assmann, G.: Tangier disease is caused by mutations in the gene encoding ATP-binding cassette transporter 1. Nature Genet. 22: 352-355, 1999.
Young, S. G.; Fielding, C. J.: The ABCs of cholesterol efflux. Nature Genet. 22: 316-318, 1999.
Delpire, E.; Lu, J.; England, R.; Dull, C.; Thorne, T.: Deafness and imbalance associated within activation of the secretory Na—K-2Cl co-transporter. Nature Genet. 22: 192-195, 1999.
Evans, R. L.; Park, K.; Turner, R. J.; Watson, G. E.; Nguyen, H.-V.; Dennett, M. R.; Hand, A. R.; Flagella, M.; Shull, G. E.; Melvin, J. E.: Severe impairment of salivation in Na+/K+/2Cl− cotransporter (NKCC1)-deficient mice. J. Biol. Chem. 275: 26720-26726, 2000.
Payne, J. A.; Xu, J.-C.; Haas, M.; Lytle, C. Y.; Ward, D.; Forbush, B., III: Primary structure, functional expression, and chromosomal localization of the bumetanide-sensitive Na—K—Cl cotransporter in human colon. J. Biol. Chem. 270: 17977-17985, 1995
Quaggin, S. E.; Payne, J. A.; Forbush, B., III; Igarashi, P.: Localization of the renal Na—K—Cl cotransporter gene (Slc12a1) on mouse chromosome 2. Mammalian Genome 6: 557-561, 1995.
Xu, J.-C.; Lytle, C.; Zhu, T. T.; Payne, J. A.; Benz, E., Jr.; Forbush, B., III: Molecular cloning and functional expression of the bumetanide-sensitive Na—K—Cl cotransporter. Proc. Nat. Acad. Sci. 91: 2201-2205, 1994.
Sedlacek, Z.; Konecki, D. S.; Korn, B.; Klauck, S. M.; Poustka, A.: Evolutionary conservation and genomic organization of XAP-4, an Xq28 located gene coding for a human rab GDP-dissociation inhibitor (GDI). Mammalian Genome 5: 633-639, 1995.
Sedlacek, Z.; Munstermann, E.; Mincheva, A.; Lichter, P.; Poustka, A.: The human rab GDI beta gene with long retroposon-rich introns maps to 10p15 and its pseudo gene to 7p11-p13. Mammalian Genome 9:78-80, 1998.
Cheng, Y.; Austin, S. C.; Rocca, B.; Koller, B. H.; Coffman, T. M.; Grosser, T.; Lawson, J. A.; FitzGerald, G. A.: Role of prostacyclin in the cardiovascular response to thromboxane A2. Science 296: 539-541, 2002.
Ueno, S.; Maruki, Y.; Nakamura, M.; Tomemori, Y.; Kamae, K.; Tanabe, H.; Yamashita, Y.; Matsuda, S.; Kaneko, S.; Sano, A.: The gene encoding a newly discovered protein, chorein, is mutated in chorea-acanthocytosis. Nature Genet. 28: 121-122, 2001.
Leone, G.; Sears, R.; Huang, E.; Rempel, R.; Nuckolls, F.; Park, C.-H.; Giangrande, P.; Wu, L.; Saavedra, H. I.; Field, S. J.; Thompson, M. A.; Yang, H.; Fujiwara, Y.; Greenberg, M. E.; Orkin, S.; Smith, C.; Nevins, J. R.: Myc requires distinct E2F activities to induce S phase and apoptosis. Molec. Cell 8: 105-113, 2001.
Cheng, S. Y.; Gong, Q. H.; Parkinson, C.; Robinson, E. A.; Appella, E.; Merlino, G. T.; Pastan, I.: The nucleotide sequence of a human cellular thyroid hormone-binding protein present in endoplasmic reticulum. J. Biol. Chem. 262: 11221-11227, 1987.
Koivu, J.; Myllyla, R.; Halaakoski, T.; Pihlajaniemi, T.; Tasanen, K.; Kivirikko, K. I.: A single polypeptide acts both as the beta-subunit of prolyl 4-hydroxylase and as a protein disulfide-isomerase. J. Biol. Chem. 262: 6447-6449, 1987.
Morris, J. I.; Varandani, P. T.: Characterization of a cDNA for human glutathione-insulin transhydrogenase (protein-disulfide isomerase/oxidoreductase). Biochim. Biophys. Acta 949: 169-180, 1988.
Noiva, R.; Lennarz, W. J.: Protein disulfide isomerase: a multifunctional protein resident in the lumen of the endoplasmic reticulum. J. Biol. Chem. 267: 3553-3556, 1992.
Pajunen, L.; Hoyhtya, M.; Tryggvason, K.; Kivirikko, K. I.; Myllyla, R.: Species-specific antibodies in the assignment of the gene for the beta-subunit of human prolyl 4-hydroxylase. (Abstract) Cytogenet. Cell Genet. 40: 719 only, 1985.
Pajunen, L.; Jones, T. A.; Goddard, A.; Sheer, D.; Solomon, E.; Pihlajaniemi, T.; Kivirikko, K. I.: Regional assignment of the human gene coding for a multifunctional polypeptide (P4HB) acting as the beta-subunit of prolyl 4-hydroxylase and the enzyme protein disulfide isomerase to 17q25. Cytogenet. Cell Genet. 56: 165-168, 1991.
Pajunen, L.; Myllyla, R.; Helaakoski, T.; Pihlajaniemi, T.; Tasanen, K.; Hoyhtya, M.; Tryggvason, K.; Solomon, E.; Kivirikko, K. I.: Assignment of the gene coding for both the beta-subunit of prolyl 4-hydroxylase and protein disulphide isomerase to human chromosome region 17q23-25. (Abstract) Cytogenet. Cell Genet. 46: 674 only, 1987.
Pajunen, L.; Myllyla, R.; Helaakoski, T.; Pihlajaniemi, T.; Tasanen, K.; Hoyhtya, M.; Tryggvason, K.; Solomon, E.; Kivirikko, K. I.: Assignment of the gene coding for both the beta-subunit of prolyl 4-hydroxylase and the enzyme disulfide isomerase to human chromosome region 17p11-qter. Cytogenet. Cell Genet. 47: 37-41, 1988.
Pihlajaniemi, T.; Helaakoski, T.; Tasanen, K.; Myllyla, R.; Huhtala, M.-L.; Koivu, J.; Kivirikko, K. I.: Molecular cloning of the beta-subunit of human prolyl 4-hydroxylase: this subunit and protein disulphide isomerase are products of the same gene. EMBO J. 6: 643-649, 1987.
Popescu, N. C.; Cheng, S.; Pastan, I.: Chromosomal localization of the gene for a human thyroid hormone-binding protein. Am. J. Hum. Genet. 42: 560-564, 1988.
Tasanen, K.; Parkkonen, T.; Chow, L. T.; Kivirikko, K. I.; Pihlajaniemi, T.: Characterization of the human gene for a polypeptide that acts both as the beta-subunit of prolyl 4-hydroxylase and as protein disulfide isomerase. J. Biol. Chem. 263: 16218-16224, 1988.
Kelly, A.; Powis, S. H.; Glynne, R.; Radley, E.; Beck, S.; Trowsdale, J.: Second proteasome-related gene in the human MHC class II region. Nature 353:667-668, 1991.
Martinez, C. K.; Monaco, J. J.: Homology of proteasome subunits to a major histocompatibility complex-linked LMP gene. Nature 353:664-667, 1991.
Petes, T. D.: Meiotic recombination hot spots and cold spots. Nature Rev. Genet. 2: 360-369, 2001.
Van Kaer, L.; Ashton-Rickardt, P. G.; Eichelberger, M.; Gaczynska, M.; Nagashima, K.; Rock, K. L.; Goldberg, A. L.; Doherty, P. C.; Tonegawa, S.: Altered peptidase and viral-specific T cell response in LMP2 mutant mice. Immunity 1: 533-541, 1994.
Zhou, P.; Zanelli, E.; Smart, M.; David, C.: Genomic organization and tissue expression of mouse proteasome gene Lmp-2. Genomics 16:664-668, 1993.
Doolittle, R. F.; Hunkapiller, M. W.; Hood, L. E.; Devare, S. G.; Robbins, K. C.; Aaronson, S. A.; Antoniades, H. N.: Simian sarcoma virus one gene, v-sis, is derived from the gene (or genes) encoding a platelet-derived growth factor. Science 221: 275-277, 1983.
Gardner, T. L.; Elston, D. M.; Wotowic, P. J.: A familial dermatofibrosarcoma protuberans. J. Am. Acad. Derm. 39: 504-505, 1998.
Groffen, J.; Heisterkamp, N.; Stephenson, J. R.; Geurts van Kessel, A.; de Klein, A.; Grosveld, G.; Bootsma, D.: c-sis is translocated from chromosome 22 to chromosome 9 in chronic myelocytic leukemia. J. Exp. Med. 158: 9-15, 1983.
Hermansson, M.; Nister, M.; Betsholtz, C.; Heldin, C.-H.; Westermark, B.; Funa, K.: Endothelial cell hyperplasia in human glioblastoma: coexpression of mRNA for platelet-derived growth factor (PDGF) B chain and PDGF receptor suggests autocrine growth stimulation. Proc. Nat. Acad. Sci. 85: 7748-7752, 1988.
Josephs, S. F.; Dalla-Favera, R.; Gelmann, E. P.; Gallo, R. C.; Wong-Staal, F.: 5-prime viral and human cellular sequences corresponding to the transforming gene of simian sarcoma virus. Science 219: 503-505, 1983.
Josephs, S. F.; Guo, C.; Ratner, L.; Wong-Staal, F.: Human proto-oncogene nucleotide sequences corresponding to the transforming region of simian sarcoma virus. Science 223: 487-491, 1984.
Josephs, S. F.; Ratner, L.; Clarke, M. F.; Westin, E. H.; Reitz, M. S.; Wong-Staal, F.: Transforming potential of human c-sis nucleotide sequences encoding platelet-derived growth factor. Science 225:636-639, 1984.
Kelly, J. D.; Raines, E. W.; Ross, R.; Murray, M. J.: The B chain of PDGF alone is sufficient for mitogenesis. EMBO J. 4: 3399-3405, 1985.
Kiuru-Kuhlefelt, S.; El-Rifai, W.; Fanburg-Smith, J.; Kere, J.; Miettinen, M.; Knuutila, S.: Concomitant DNA copy number amplification at 17q and 22q in dermatofibrosarcoma protuberans. Cytogenet. Cell Genet. 92: 192-195, 2001.
Kozak, C. A.; Sears, J. F.; Hoggan, M. D.: Genetic mapping of the mouse proto-oncogene c-sis to chromosome 15. Science 221: 867-869, 1983.
Lindahl, P.; Johansson, B. R.; Leveen, P.; Betsholtz, C.: Pericyte loss and microaneurysm formation in PDGFB-deficient mice. Science 277:242-245, 1997.
Owen, A. J.; Pantazis, P.; Antoniades, H. N.: Simian sarcoma virus-transformed cells secrete a mitogen identical to platelet-derived growth factor. Science 225: 54-56, 1984.
Rao, C. D.; Igarashi, H.; Chiu, I.-M.; Robbins, K. C.; Aaronson, S. A.: Structure and sequence of the human csis/platelet-derived growth factor 2 (SIS/PDGF2) transcriptional unit. Proc. Nat. Acad. Sci. 83: 2392-2396, 1986.
Robbins, K. C.; Antoniades, H. N.; Devare, S. G.; Hunkapiller, M. W.; Aaronson, S. A.: Structural and immunological similarities between simian sarcoma virus gene product(s) and human platelet-derived growth factor. Nature 305: 605-608, 1983.
Robbins, K. C.; Devare, S. G.; Reddy, E. P.; Aaronson, S. A.: In vivo identification of the transforming gene product of simian sarcoma virus. Science 218: 1131-1133, 1982.
Simon, M.-P.; Navarro, M.; Roux, D.; Pouyssegur, J.: Structural and functional analysis of a chimeric protein COL1A1-PDGFB generated by the translocation t(17;22)(q22;q13.1) in dermatofibrosarcoma protuberans (DP). Oncogene 20: 2965-2975, 2001.
Smidt, M.; Kirsch, I.; Ratner, L.: Deletion of Alu sequences in the fifth c-sis intron in individuals with meningiomas. J. Clin. Invest. 86: 1151-1157, 1990.
Turc-Carel, C.; Philip, I.; Berger, M. P.; Philip, T.; Lenoir, G. M.: Chromosomal translocations in Ewing's sarcoma. (Letter) New Eng. J. Med. 309: 497-498, 1983.
Shimizu, A.; O'Brien, K. P.; Sjoblom, T.; Pietras, K.; Buchdunger, E.; Collins, V. P.; Heldin, C.-H.; Dumanski, J. P.; Ostman, A.: The dermatofibrosarcoma protuberans-associated collagen type I-alpha-1/platelet-derived growth factor (PDGF) beta-chain fusion gene generates a transforming protein that is processed to functional PDGF-BB. Cancer Res. 59:3719-3723, 1999.
Waterfield, M. D.; Scrace, G. T.; Whittle, N.; Stroobant, P.; Johnsson, A.; Wasteson, A.; Westermark, B.; Heldin, C.-H.; Huang, J. S.; Deuel, T. F.: Platelet-derived growth factor is structurally related to the putative transforming protein p28(sis) of simian sarcoma virus. Nature 304: 35-39, 1983
Simon, M.-P.; Pedeutour, F.; Sirvent, N.; Grosgeorge, J.; Minoletti, F.; Coindre, J.-M.; Terrier-Lacombe, M.-J.; Mandahl, N.; Craver, R. D.; Blin, N.; Sozzi, G.; Turc-Carel, C.; O'Brien, K. P.; Kedra, D.; Fransson, I.; Guilbaud, C.; Dumanski, J. P.: Deregulation of the platelet-derived growth factor B-chain gene via fusion with collagen gene COL1A1 in dermatofibrosarcoma protuberans and giant-cell fibroblastoma. Nature Genet. 15: 95-98, 1997.
Gimona, M.; Small, J. V.; Moeremans, M.; Van Damme, J.; Puype, M.; Vandekerckhove, J.: Porcine vinculin and metavinculin differ by a 68-residue insert located close to the carboxy-terminal part of the molecule. EMBO J. 7: 2329-2334, 1988.
Lee, F. S.; Fox, E. A.; Zhou, H.-M.; Strydom, D. J.; Vallee, B. L.: Primary structure of human placental ribonuclease inhibitor. Biochemistry 27:8545-8553, 1988.
Weremowicz, S.; Fox, E. A.; Morton, C. C.; Vallee, B. L.: The placental ribonuclease inhibitor (RNH) gene is located on chromosome subband 11p15.5. Genomics 8: 717-721, 1990.
Zneimer, S. M.; Crawford, D.; Schneider, N. R.; Beutler, B.: Mapping of the human ribonuclease inhibitor gene (RNH) to chromosome 11p15 by in situ hybridization. Genomics 8: 175-178, 1990.
Klingensmith, J.; Nusse, R.; Perrimon, N.: The Drosophila segment polarity gene dishevelled encodes a novel protein required for response to wingless signal. Genes Dev. 8: 118-130, 1994.
Bui, T. D.; Beier, D. R.; Jonssen, M.; Smith, K.; Dorrington, S. M.; Kaklamanis, L.; Kearney, L.; Regan, R.; Sussman, D. J.; Harris, A. L.: cDNA cloning of a human dishevelled DVL-3 gene, mapping to 3q27, and expression in human breast and colon carcinomas. Biochem. Biophys. Res. Commun. 239: 510-516, 1997.
Cheng, Y.-S. E.; Patterson, C. E.; Staeheli, P.: Interferon-induced guanylate-binding proteins lack an N(T)KXD consensus motif and bind GMP in addition to GDP and GTP. Molec. Cell. Biol. 11: 4717-4725, 1991.
Kumar, S.; Li, Q.; Dua, A.; Ying, Y.-K.; Bagchi, M. K.; Bagchi, I. C.: Messenger ribonucleic acid encoding interferon-inducible guanylate binding protein 1 is induced in human endometrium within the putative window of implantation. J. Clin. Endocr. Metab. 86: 2420-2427, 2001.
Prochazka, M.; Staeheli, P.; Holmes, R. S.; Haller, O.: Interferon-induced guanylate-binding proteins: mapping of the murine Gbp-1 locus to chromosome 3. Virology 145: 273-279, 1985.
Strehlow, I.; Lohmann-Matthes, M. L.; Decker, T.: The interferon-inducible GBP1 gene: structure and mapping to human chromosome 1. Gene 144:295-299, 1994.
Aoki, K.; Suzuki, K.; Sugano, T.; Tasaka, T.; Nakahara, K.; Kuge, O.; Omori, A.; Kasai, M.: A novel gene, ‘Translin,’ encodes a recombination hotspot binding protein associated with chromosomal translocations. Nature Genet. 10: 167-174, 1995.
Badge, R. M.; Yardley, J.; Jeffreys, A. J.; Armour, J. A. L.: Crossover breakpoint mapping identifies a subtelomeric hotspot for male meiotic recombination. Hum. Molec. Genet. 9: 1239-1244, 2000.
Hosaka, T.; Kanoe, H.; Nakayama, T.; Murakami, H.; Yamamoto, H.; Nakamata, T.; Tsuboyama, T.; Oka, M.; Kasai, M.; Sasaki, M. S.; Nakamura, T.; Toguchida, J.: Translin binds to the sequences adjacent to the breakpoints of the TLS and CHOP genes in liposarcomas with translocationt (12;16). Oncogene 19: 5821-5825, 2000.
Kasai, M.; Aoki, K.; Matsuo, Y.; Minowada, J.; Maziarz, R. T.; Strominger, J. L.: Recombination hotspot associated factors specifically recognize novel target sequences at the site of interchromosomal rearrangements in T-ALL patients with t(8;14)(q24;q11) and (t(1;14)(p32;q11). Int. Immun. 6: 1017-1025, 1994.
Cirillo, L. A.; Lin, F. R.; Cuesta, I.; Friedman, D.; Jarnik, M.; Zaret, K. S.: Opening of compacted chromatin by early developmental transcription factors HNF3(FoxA) and GATA-4. Molec. Cell 9: 279-289, 2002.
Braun-Dullaeus, R. C.; Mann, M. J.; Ziegler, A.; von der Leyen, H. E.; Dzau, V. J.: A novel role for the cyclin-dependent kinase inhibitor p27(Kip1) in angiotensin II-stimulated vascular smooth muscle cell hypertrophy. J. Clin. Invest. 104: 815-823, 1999.
Carrano, A. C.; Eytan, E.; Hershko, A.; Pagano, M.: SKP2 is required for ubiquitin-mediated degradation of the CDK inhibitor p27. Nature Cell Biol. 1: 193-199, 1999.
Di Cristofano, A.; De Acetis, M.; Koff, A.; Cordon-Cardo, C.; Pandolfi, P. P.: Pten and p27(KIP1) cooperate in prostate cancer tumor suppression in the mouse. Nature Genet. 27: 222-224, 2001.
Fero, M. L.; Rivkin, M.; Tasch, M.; Porter, P.; Carow, C. E.; Firpo, E.; Polyak, K.; Tsai, L.-H.; Broudy, V.; Perlmutter, R. M.; Kaushansky, K.; Roberts, J. M.: A syndrome of multiorgan hyperplasia with features of gigantism, tumorigenesis, and female sterility in p27(Kip1)-deficient mice. Cell 85: 733-744, 1996.
Buyse, I. M.; Shao, G.; Huang, S.: The retinoblastoma protein binds to RIZ, a zinc-finger protein that shares an epitope with the adenovirus E1A protein. Proc. Nat. Acad. Sci. 92: 4467-4471, 1995.
Buyse, I. M.; Takahashi, E.; Huang, S.: Physical mapping of the retinoblastoma interacting zinc finger gene RIZ to D1S228 on chromosome 1p36. Genomics 34: 119-121, 1996.
Chadwick, R. B.; Jiang, G.-L.; Bennington, G. A.; Yuan, B.; Johnson, C. K.; Stevens, M. W.; Niemann, T. H.; Peltomaki, P.; Huang, S.; dela Chapelle, A.: Candidate tumor suppressor RIZ is frequently involved in colorectal carcinogenesis. Proc. Nat. Acad. Sci. 97: 2662-2667, 2000.
Mock, B. A.; Coleman, M. P.; Huang, S.: R1z maps to distal chromosome 4 near genes involved in tumorigenesis and nerve degeneration. Mammalian Genome 7: 637 only, 1996.
Poetsch, M.; Dittberner, T.; Woenckhaus, C.: Frameshift mutations of RIZ, but no point mutations in RIZ1 exons in malignant melanomas with deletions in 1p36. Oncogene 21: 3038-3042, 2002.
Malek, N. P.; Sundberg, H.; McGrew, S.; Nakayama, K.; Kyriakidis, T. R.; Roberts, J. M.: A mouse knock-in model exposes sequential proteolytic pathways that regulate p27(Kip1) in G1 and S phase. Nature 413:323-327, 2001.
Martin, E.; Cacheux, V.; Cave, H.; Lapierre, J. M.; Le\Paslier, D.; Grandchamp, B.: Localization of the CDKN4/p27(Kip1) gene to human chromosome 12p12.3. Hum. Genet. 96: 668-670, 1995.
Mitsuhashi, T.; Aoki, Y.; Eksioglu, Y. Z.; Takahashi, T.; Bhide, P. G.; Reeves, S. A.; Caviness, V. S., Jr.: Overexpression of p27(Kip1) lengthens the G1 phase in a mouse model that targets inducible gene expression to central nervous system progenitor cells. Proc. Nat. Acad. Sci. 98: 6435-6440, 2001.
Polyak, K.; Lee, M.-H.; Erdjument-Bromage, H.; Koff, A.; Roberts, J. M.; Tempst, P.; Massague, J.: Cloning of p27(Kip1), a cyclin-dependent kinase inhibitor and a potential mediator of extracellular antimitogenic signals. Cell 78: 59-66, 1994.
Saito, T.; Seki, N.; Hattori, A.; Hayashi, A.; Abe, M.; Araki, R.; Fujimori, A.; Fukumura, R.; Kozuma, S.; Matsuda, Y.: Structure, expression profile, and chromosomal location of a mouse gene homologous to human DNA-PK(cs) interacting protein (KIP) gene. Mammalian Genome 10:315-317, 1999.
Sherr, C. J.; Roberts, J. M.: Inhibitors of mammalian G1 cyclin-dependent kinases. Genes Dev. 9: 1149-1163, 1995.
Toyoshima, H.; Hunter, T.: p27, a novel inhibitor of G1 cyclin-Cdk protein kinase activity, is related to p21. Cell 78: 67-74, 1994.
Druck, T.; Gu, Y.; Prabhala. G.; Cannizzaro, L. A.; Park, S.-H.; Huebner, K.; Keen, J. H.: Chromosome localization of human genes for clathrin adaptor polypeptides AP2-beta and AP50 and the clathrin-binding protein, VCP. Genomics 30: 94-97, 1995.
Ponnambalam, S.; Robinson, M. S.; Jackson, A. P.; Peiperl, L.; Parham, P.: Conservation and diversity in families of coated vesicle adaptins. J. Biol. Chem. 265: 4814-4820, 1990.
Kaneko-Ishino, T.; Kuroiwa, Y.; Miyoshi, N.; Kohda, T.; Suzuki, R.; Yokoyama, M.; Viville, S.; Barton, S. C.; Ishino, F.; Surani, M. A.: Peg1/Mest imprinted gene on chromosome 6 identified by cDNA subtraction hybridization. Nature Genet. 11: 52-59, 1995.
Kobayashi, S.; Kohda, T.; Miyoshi, N.; Kuroiwa, Y.; Aisaka, K.; Tsutsumi, O.; Kaneko-Ishino, T.; Ishino, F.: Human PEG1/MEST, an imprinted gene on chromosome 7. Hum. Molec. Genet. 6: 781-786, 1997.
Kosaki, K.; Kosaki, R.; Craigen, W. J.; Matsuo, N.: Isoform-specific imprinting of the human PEG1/MEST gene. (Letter) Am. J. Hum. Genet. 66:309-312, 2000.
Kotzot, D.; Schmitt, S.; Bernasconi, F.; Robinson, W. P.; Lurie, I. W.; Ilyina, H.; Mehes, K.; Hamel, B. C. J.; Otten, B. J.; Hergersberg, M.; Werder, E.; Shoenle, E.; Schinzel, A.: Uniparental disomy 7 in Silver-Russell syndrome and primordial growth retardation. Hum. Molec. Genet. 4: 583-587, 1995.
Lefebvre, L.; Viville, S.; Barton, S. C.; Ishino, F.; Keverne, E. B.; Surani, M. A.: Abnormal maternal behaviour and growth retardation associated with loss of the imprinted gene Mest. Nature Genet. 20:163-169, 1998.
Lefebvre, L.; Viville, S.; Barton, S. C.; Ishino, F.; Surani, M. A.: Genomic structure and parent-of-origin-specific methylation of Peg1. Hum. Molec. Genet. 6: 1907-1915, 1997.
Ayala-Madrigal, M. L.; Doerr, S.; Ramirez-Duenas, M. L.; Hansmann, I.: Assignment of karyopherin alpha 1 (KPNA1) to human chromosome band 3q21 by in situ hybridization. Cytogenet. Cell Genet. 90: 58-59, 2000.
Conti, E.; Uy, M.; Leighton, L.; Blobel, G.; Kuriyan, J.: Crystallographic analysis of the recognition of a nuclear localization signal by the nuclear import factor karyopherin alpha. Cell 94: 193-204, 1998.
Cortes, P.; Ye, Z.-S.; Baltimore, D.: RAG-1 interacts with the repeated amino acid motif of the human homologue of the yeast protein SRP1. Proc. Nat. Acad. Sci. 91: 7633-7637, 1994.
Barichard, F.; Joulin, V.; Henry, I.; Garel, M.-C.; Valentin, C.; Rosa, R.; Cohen-Solal, M.; Junien, C.: Chromosomal assignment of the human 2,3-bisphosphoglycerate mutase gene (BPGM) to region 7q34-7q22. Hum. Genet. 77: 283-285, 1987.
Bowdler, A. J.; Prankerd, T. A. J.: Studies in congenital non-spherocytichaemolytic anaemias with specific enzyme defects. Acta Haemat. 31:65-78, 1964.
Chen, S.-H.; Anderson, J. E.; Giblett, E. R.: 2,3-diphosphoglyceratemutase: its demonstration by electrophoresis and the detection of a genetic variant. Biochem. Genet. 5: 481-486, 1971.
Galacteros, F.; Rosa, R.; Prehu, M. O.; Najean, Y.; Calvin, M. C.: Deficit en diphosphoglycerate mutase: nouveaux cas associes aune polyglobulie. Nouv. Rev. Franc. Hemat. 26: 69-74, 1984
Joulin, V.; Barichard, F.; Henry, I.; Garel, M. C.; Valentin, C.; Rosa, R.; Cohen-Solal, M.; Junien, C.: Chromosomal assignment of the human 2,3-bisphosphoglycerate mutase gene (BPGM) to region 7q22-7q34. (Abstract) Cytogenet. Cell Genet. 46: 635 only, 1987.
Joulin, V.; Garel, M.-C.; Le Boulch, P.; Valentin, C.; Rosa, R.; Rosa, J.; Cohen-Solal, M.: Isolation and characterization of the human 2,3-bisphosphoglycerate mutase gene. J. Biol. Chem. 263: 15785-15790, 1988.
Joulin, V.; Peduzzi, J.; Romeo, P.-H.; Rosa, R.; Valentin, C.; Dubart, A.; Lapeyre, B.; Blouquit, Y.; Garel, M.-C.; Goossens, M.; Rosa, J.; Cohen-Solal, M.: Molecular cloning and sequencing of the human erythrocyte 2,3-bisphosphoglycerate mutase cDNA: revised amino acid sequence. EMBO J. 5: 2275-2283, 1986.
Labie, D.; Leroux, J.-P.; Najman, A.; Reyrolle, C.: Familial diphosphoglyceratemutase deficiency: influence on the oxygen affinity curves of hemoglobin. FEBS Lett. 9: 37-40, 1970.
Lemarchandel, V.; Joulin, V.; Valentin, C.; Rosa, R.; Galacteros, F.; Rosa, J.; Cohen-Solal, M.: Compound heterozygosity in a complete erythrocyte bisphosphoglycerate mutase deficiency. Blood 80: 2643-2649, 1992
Rosa, R.; Audit, I.; Rosa, J.: Diphosphoglycerate mutase and 2,3-diphosphoglycerate phosphatase activities of red cells: comparative electrophoretic study. Biochem. Biophys. Res. Commun. 51: 536-542, 1973.
Rosa, R.; Blouquit, Y.; Calvin, M.-C.; Prome, D.; Prome, J.-C.; Rosa, J.: Isolation, characterization, and structure of a mutant 89 arg-to-cys bisphosphoglycerate mutase: implication of the active site in the mutation. J. Biol. Chem. 264: 7837-7843, 1989.
Rosa, R.; Prehu, M.-O.; Beuzard, Y.; Rosa, J.: The first case of a complete deficiency of diphosphoglycerate mutase in human erythrocytes. J. Clin. Invest. 62: 907-915, 1978.
Sasaki, R.; Ikura, K.; Sugimoto, E.; Chiba, H.: Purification of bisphosphoglyceromutase, 2,3-bisphosphoglycerate phosphatase and phosphoglyceromutase from human erythrocytes. Europ. J. Biochem. 50:581-593, 1975.
Schroter, W.: Kongenitale nichtsphaerocytaere haemolytische Anaemiebei 2,3-Diphosphoglyceratmutasemangel der Erythrocyten im fruehenSaeuglingsalter. Klin. Wschr. 43: 1147-1153, 1965.
Scott, E. M.; Wright, R. C.: An alternate method for demonstration of bisphosphoglyceromutase (DPGM) on starch gels. Am. J. Hum. Genet. 34:1013-1015, 1982.
Yanagawa, S.; Hitomi, K.; Sasaki, R.; Chiba, H.: Isolation and characterization of cDNA encoding rabbit reticulocyte 2,3-bisphosphoglycerate synthase. Gene 44: 185-191, 1986.
Hiesberger, T.; Trommsdorff, M.; Howell, B. W.; Goffinet, A.; Mumby, M. C.; Cooper, J. A.; Herz, J.: Direct binding of reelin to VLDL receptor and apoE receptor 2 induces tyrosine phosphorylation of disabled-1 and modulates tau phosphorylation. Neuron 24: 481-489, 1999.
Trommsdorff, M.; Gotthardt, M.; Hiesberger, T.; Shelton, J.; Stockinger, W.; Nimpf, J.; Hammer, R. E.; Richardson, J. A.; Herz, J.: Reeler/Disabled-like disruption of neuronal migration in knockout mice lacking the VLDL receptor and ApoE receptor 2. Cell 97: 689-701, 1999.
Huebner, K.; Druck, T.; Croce, C. M.; Thiesen, H. J.: Twenty-seven nonoverlapping zinc finger cDNAs from human T cells map to nine different chromosomes with apparent clustering. Am. J. Hum. Genet. 48: 726-740, 1991.
Rousseau-Merck, M.-F.; Hillion, J.; Jonveaux, P.; Couillin, P.; Seite, P.; Thiesen, H.-J.; Berger, R.: Chromosomal localization of 9 KOX zinc finger genes: physical linkages suggest clustering of KOX genes on chromosomes 12, 16, and 19. Hum. Genet. 92: 583-587, 1993.
Lehmann, O. J.; El-ashry, M. F.; Ebenezer, N. D.; Ocaka, L.; Francis, P. J.; Wilkie, S. E.; Patel, R. J.; Ficker, L.; Jordan, T.; Khaw, P. T.; Bhattacharya, S. S.: A novel keratocan mutation causing autosomal recessive cornea plana. Invest. Ophthal. Vis. Sci. 42: 3118-3122, 2001.
DiLella, A. G.: Chromosomal assignment of the human immunophilin FKBP-12 gene. Biochem. Biophys. Res. Commun. 179: 1427-1433, 1991.
DiLella, A. G.; Hawkins, A.; Craig, R. J.; Schreiber, S. L.; Griffin, C. A.: Chromosomal band assignments of the genes encoding human FKBP12 and FKBP13. Biochem. Biophys. Res. Commun. 189: 819-823, 1992.
Goebl, M. G.: The peptidyl-prolyl isomerase, FK506-binding protein, is most likely the 12 kd endogenous inhibitor 2 of protein kinase C. (Letter) Cell 64: 1051-1052, 1991.
Jin, Y.-J.; Albers, M. W.; Lane, W. S.; Bierer, B. E.; Schreiber, S. L.; Burakoff, S. J.: Molecular cloning of a membrane-associated human FK506- and rapamycin-binding protein, FKBP-13. Proc. Nat. Acad. Sci. 88: 6677-6681, 1991.
Maki, N.; Sekiguchi, F.; Nishimaki, J.; Miwa, K.; Hayano, T.; Takahashi, N.; Suzuki, M.: Complementary DNA encoding the human T-cell FK506-binding protein, a peptidylprolyl cis-trans isomerase distinct from cyclophilin. Proc. Nat. Acad. Sci. 87: 5440-5443, 1990.
Peattie, D. A.; Hsaio, K.; Benasutti, M.; Lippke, J. A.: Three distinct messenger RNAs can encode the human immunosuppressant-binding protein FKBP12. Gene 150: 251-257, 1994.
Shou, W.; Aghdasi, B.; Armstrong, D. L.; Guo, Q.; Bao, S.; Charng, M.-J.; Mathews, L. M.; Schneider, M. D.; Hamilton, S. L.; Matzuk, M. M.: Cardiac defects and altered ryanodine receptor function in mice lacking FKBP12. Nature 391: 489-492, 1998.
Standaert, R. F.; Galat, A.; Verdine, G. L.; Schreiber, S. L.: Molecular cloning and overexpression of the human FK506-binding protein FKBP. Nature 346: 671-674, 1990.
Wang, T.; Donahoe, P. K.; Zervos, A. S.: Specific interaction of type I receptors of the TGF-beta family with the immunophilin FKBP-12. Science 265:674-676, 1994.
Martell, K. J.; Kwak, S.; Hakes, D. J.; Dixon, J. E.; Trent, J. M.: Chromosomal localization of four human VH1-like protein-tyrosine phosphatases. Genomics 22: 462-464, 1994
Bogenmann, E.; Lochrie, M. A.; Simon, M. I.: Cone cell-specific genes expressed in retinoblastoma. Science 240: 76-78, 1988.
Bonaiti-Pellie, C.; Briard-Guillemot, M. L.: Segregation analysis in hereditary retinoblastoma. Hum. Genet. 57: 411-419, 1981.
Hunter, A. G. W.; Thompson, M. W.: Acromesomelic dwarfism: description of a patient and comparison with previously reported cases. Hum. Genet. 34: 107-113, 1976.
Miceli-Richard, C.; Lesage, S.; Rybojad, M.; Prieur, A.-M.; Manouvrier-Hanu, S.; Hafner, R.; Chamaillard, M.; Zouali, H.; Thomas, G.; Hugot, J.-P.: CARD15 mutations in Blau syndrome. Nature Genet. 29: 19-20, 2001.
Han, L. Wong, D.; Dhaka, A.; Afar, D.; White, M.; Xie, W.; Herschman, H.; Witte, O.: Colicelli, J.: Protein binding and signaling properties of RIN1 suggest a unique effector function. Proc. Nat. Acad. Sci. 94:4954-4959, 1997.
Jhanwar, S. C.; Neel, B. G.; Hayward, W. S.; Chaganti, R. S. K.: Localization of the cellular oncogenes ABL, SIS, and FES on human germ-line chromosomes. Cytogenet. Cell Genet. 38: 73-75, 1984.
Bao, S.; Tibbetts, R. S.; Brumbaugh, K. M.; Fang, Y.; Richardson, D. A.; Ali, A.; Chen, S. M.; Abraham, R. T.; Wang, X.-F.: ATR/ATM-mediated phosphorylation of human Rad17 is required for genotoxic stress responses. Nature 411:969-974, 2001.
Goedert, M.; Hasegawa, J.; Craxton, M.; Leversha, M. A.; Clegg, S.: Assignment of the human stress-activated protein kinase-3 gene (SAPK3) to chromosome 22q13.3 by fluorescence in situ hybridization. Genomics 41:501-502, 1997.
Avraham, K. B.; Prezioso, V. R.; Chen, W. S.; Lai, E.; Sladek, F. M.; Zhong, W.; Darnell, J. E., Jr.; Jenkins, N. A.; Copeland, N. G.: Murine chromosomal location of four hepatocyte-enriched transcription factors: HNF-3-alpha, HNF-3-beta, HNF-3-gamma, and HFN-4. Genomics 13:264-268, 1992.
Kaestner, K. H.; Hiemisch, H.; Luckow, B.; Schutz, G.: The HNF-3 gene family of transcription factors in mice: gene structure, cDNA sequence, and mRNA distribution. Genomics 20: 377-385, 1994.
Mincheva, A.; Lichter, P.; Schutz, G.; Kaestner, K. H.: Assignment of the human genes for hepatocyte nuclear factor 3-alpha, -beta, and-gamma (HNF3A, HNF3B, HNF3G) to 14q12-q13, 20p11, and 19q13.2-q13.4. Genomics 39:417-419, 1997.
Adams, R. H.; Porras, A.; Alonso, G.; Jones, M.; Vintersten, K.; Panelli, S.; Valladares, A.; Perez, L.; Klein, R.; Nebreda, A. R.: Essential role of p38-alpha MAP kinase in placental but not embryonic cardiovascular development. Molec. Cell 6: 109-116, 2000.
Ge, B.; Gram, H.; Di Padova, F.; Huang, B.; New, L.; Ulevitch, R. J.; Luo, Y.; Han, J.: MAPKK-independent activation of p38-alpha mediated by TAB1-dependent autophosphorylation of p38-alpha. Science 295:1291-1294, 2002.
Han, J.; Lee, J.-D.; Bibbs, L.; Ulevitch, R. J.: A MAP kinase-targeted by endotoxin and hyperosmolarity in mammalian cells. Science 265:808-811, 1994.
Kim, D. H.; Feinbaum, R.; Alloing, G.; Emerson, F. E.; Garsin, D. A.; Inoue, H.; Tanaka-Hino, M.; Hisamoto, N.; Matsumoto, K.; Tan, M.-W.; Ausubel, F. M.: A conserved p38 MAP kinase pathway in Caenorhabditis elegans innate immunity. Science 297: 623-626, 2002.
Kumar, S.; McLaughlin, M. M.; McDonnell, P. C.; Lee, J. C.; Livi, G. P.; Young, P. R.: Human mitogen-activated protein kinase CSBP1, but not CSBP2, complements a hog1 deletion in yeast. J. Biol. Chem. 270:29043-29046, 1995.
Lee, J. C.; Laydon, J. T.; McDonnell, P. C.; Gallagher, T. F.; Kumar, S.; Green, D.; McNulty, D.; Blumenthal, M. J.; Heys, J. R.; Landvatter, S. W.; Stickler, J. E.; McLaughlin, M. M.; Siemens, I. R.; Fisher, S. M.; Livi, G. P.; White, J. R.; Adams, J. L.; Young, P. R.: A protein kinase involved in the regulation of inflammatory cytokine biosynthesis. Nature 372: 739-746, 1994.
Liao, P.; Georgakopoulos, D.; Kovacs, A.; Zheng, M.; Lerner, D.; Pu, H.; Saffitz, J.; Chien, K.; Xiao, R.-P.; Kass, D. A.; Wang, Y.: The in vivo role of p38 MAP kinases in cardiac remodeling and restrictive cardiomyopathy. Proc. Nat. Acad. Sci. 98: 12283-12288, 2001.
Maizels, E. T.; Mukherjee, A.; Sithanandam, G.; Peters, C. A.; Cottom, J.; Mayo, K. E.; Hunzicker-Dunn, M.: Developmental regulation of mitogen-activated protein kinase-activated kinases-2 and -3 (MAPKAPK-2/-3) in vivo during corpus luteum formation in the rat. Molec. Endocr. 15:716-733, 2001.
McDonnell, P. C.; DiLella, A. G.; Lee, J. C.; Young, P. R.: Localization of the human stress responsive MAP kinase-like CSAIDs binding protein (CSBP) gene to chromosome 6p21.3/21.2. Genomics 29: 301-302, 1995.
New, L.; Jiang, Y.; Zhao, M.; Liu, K.; Zhu, W.; Flood, L. J.; Kato, Y.; Parry, G. C. N.; Han, J.: PRAK, a novel protein kinase regulated by the p38 MAP kinase. EMBO J. 17: 3372-3384, 1998.
Ni, H.; Wang, X. S.; Diener, K.; Yao, Z.: MAPKAPK5, a novel mitogen-activated protein kinase (MAPK)-activated protein kinase, is a substrate of the extracellular-regulated kinase (ERK) and p38 kinase. Biochem. Biophys. Res. Commun. 243: 492-496, 1998.
Takekawa, M.; Maeda, T.; Saito, H.: Protein phosphatase 2C-alpha inhibits the human stress-responsive p38 and JNK MAPK pathways. EMBO J. 17: 4744-4752, 1998.
Tamura, K.; Sudo, T.; Senftleben, U.; Dadak, A. M.; Johnson, R.; Karin, M.: Requirement for p38-alpha in erythropoietin expression: a role for stress kinases in erythropoiesis. Cell 102: 221-231, 2000.
Haber, N.; Stengel, D.; Defer, N.; Roeckel, N.; Mattei, M.-G.; Hanoune, J.: Chromosomal mapping of human adenylyl cyclase genes type III, type V and type VI. Hum. Genet. 94: 69-73, 1994.
Anand, A.; Chada, K.: In vivo modulation of Hmgic reduces obesity. Nature Genet. 24: 377-380, 2000.
Arlotta, P.; Tai, A. K.-F.; Manfioletti, G.; Clifford, C.; Jay, G.; Ono, S. J.: Transgenic mice expressing a truncated form of the high mobility group I—C protein develop adiposity and an abnormally high prevalence of lipomas. J. Biol. Chem. 275: 14394-14400, 2000.
Ashar, H. R.; Cherath, L.; Przybysz, K. M.; Chada, K.: Genomic characterization of human HMGIC, a member of the accessory transcription factor family found at translocation breakpoints in lipomas. Genomics 31:207-214, 1996.
Ashar, H. R.; Schoenberg Fejzo, M.; Tkachenko, A.; Zhou, X.; Fletcher, J. A.; Weremowicz, S.; Morton, C. C.; Chada, K.: Disruption of the architectural factor HMGI-C: DNA-binding AT hook motifs fused in lipomas to distinct transcriptional regulatory domains. Cell 82: 57-65, 1995.
Chau, K.-Y.; Patel, U. A.; Lee, K.-L. D.; Lam, H.-Y. P.; Crane-Robinson, C.: The gene for the human architectural transcription factor HMGI-C consists of five exons each coding for a distinct functional element. Nucleic Acids Res. 23: 4262-4266, 1995.
Danforth, E., Jr.: Failure of adipocyte differentiation causes type II diabetes mellitus. Nature Genet. 26: 13 only, 2000.
Friedmann, M.; Holth, L. T.; Zoghbi, H. Y.; Reeves, R.: Organization, inducible-expression and chromosome localization of the human HMG-I(Y)nonhistone protein gene. Nucleic Acids Res. 21: 4259-4267, 1993. Ishwad, C. S.; Shriver, M. D.; Lassige, D. M.; Ferrell, R. E.: The high mobility group I—C gene (HMGI-C): polymorphism and genetic localization. Hum. Genet. 99: 103-105, 1997.
Kazmierczak, B.; Dal Cin, P.; Wanschura, S.; Bartnitzke, S.; Vanden Berghe, H.; Bullerdiek, J.: Cloning and molecular characterization of part of a new gene fused to HMGIC in mesenchymal tumors. Am. J. Path. 152: 431-435, 1998.
Kazmierczak, B.; Pohnke, Y.; Bullerdiek, J.: Fusion transcripts between the HMGIC gene and RTVL-H-related sequences in mesenchymal tumors with out cytogenetic aberrations. Genomics 38: 223-226, 1996.
Manfioletti, G.; Rustighi, A.; Mantovani, F.; Goodwin, G. H.; Giancotti, V.: Isolation and characterization of the gene coding for murine high-mobility-group protein HMGI-C. Gene 167: 249-253, 1995.
Mine, N.; Kurose, K.; Nagai, H.; Doi, D.; Ota, Y.; Yoneyama, K.; Konishi, H.; Araki, T.; Emi, M.: Gene fusion involving HMGIC is a frequent aberration in uterine leiomyomas. J. Hum. Genet. 46: 408-412, 2001.
Nucci, M. R.; Weremowicz, S.; Neskey, D. M.; Sornberger, K.; Tallini, G.; Morton, C. C.; Quade, B. J.: Chromosomal translocation t(8;12) induces aberrant HMGIC expression in aggressive angiomyxoma of the vulva. Genes Chromosomes Cancer 32: 172-176, 2001.
Seimiya, H.; Sawabe, T.; Inazawa, J.; Tsuruo, T.: Cloning, expression and chromosomal localization of a novel gene for protein tyrosine phosphatase (PTP-U2) induced by various differentiation-inducing agents. Oncogene 10:1731-1738, 1995.
Wiggins, R. C.; Wiggins, J. E.; Goyal, M.; Wharram, B. L.; Thomas, P. E.: Molecular cloning of cDNAs encoding human GLEPP1, a membrane protein tyrosine phosphatase: characterization of the GLEPP1 protein distribution in human kidney and assignment of the GLEPP1 gene to human chromosome 12p12-p13. Genomics 27: 174-181, 1995.
Jia, S.; VanDusen, W. J.; Diehl, R. E.; Kohl, N. E.; Dixon, R. A. F.; Elliston, K. O.; Stern, A. M.; Friedman, P. A.: cDNA cloning and expression of bovine aspartyl (asparaginyl) beta-hydroxylase. J. Biol. Chem. 267: 14322-14327, 1992.
Korioth, F.; Gieffers, C.; Frey, J.: Cloning and characterization of the human gene encoding aspartyl beta-hydroxylase. Gene 150:395-399, 1994.
Lavaissiere, L.; Jia, S.; Nishiyama, M.; de la Monte, S.; Stern, A. M.; Wands, J. R.; Friedman, P. A.: Overexpression of human aspartyl(asparaginyl)-beta-hydroxylase in hepatocellular carcinoma and cholangiocarcinoma. J. Clin. Invest. 98:1313-1323, 1996.
Lim, K. Y.; Hong, C.-S.; Kim, D. H.: cDNA cloning and characterization of human cardiac junctin. Gene 255: 35-42, 2000.
Scott, A. F.: Personal Communication. Baltimore, Md. Feb. 19, 2001.
Treves, S.; Feriotto, G.; Moccagatta, L.; Gambari, R.; Zorzato, F.: Molecular cloning, expression, functional characterization, chromosomal localization, and gene structure of junctate, a novel integral calcium binding protein of sarco(endo)plasmic reticulum membrane. J. Biol. Chem. 275: 39555-39568, 2000.
Wetzel, G. T.; Ding, S.; Chen, F.: Molecular cloning of junctin from human and developing rabbit heart. Molec. Genet. Metab. 69:252-258, 2000.
Vikkula, M.; Boon, L. M.; Carraway, K. L., III; Calvert, J. T.; Diamonti, A. J.; Goumnerov, B.; Pasyk, K. A.; Marchuk, D. A.; Warman, M. L.; Cantley, L. C.; Mulliken, J. B.; Olsen, B. R.: Vascular dysmorphogenesis caused by an activating mutation in the receptor tyrosine kinase TIE2. Cell 87:1181-1190, 1996.
Barker, H. M.; Brewis, N. D.; Street, A. J.; Spurr, N. K.; Cohen, P. T. W.: Three genes for protein phosphatase 1 map to different human chromosomes: sequence, expression and gene localisation of protein serine/threonine phosphatase 1 beta (PPP1CB). Biochim. Biophys. Acta 1220:212-218, 1994.
Saadat, M.; Kakinoki, Y.; Mizuno, Y.; Kikuchi, K.; Yoshida, M. C.: Chromosomal localization of human, rat, and mouse protein phosphatase type 1 beta catalytic subunit genes (PPP1CB) by fluorescence in situ hybridization. Jpn. J. Genet. 69: 697-700, 1994.
Becq, F.; Hamon, Y.; Bajetto, A.; Gola, M.; Verrier, B.; Chimini, G.: ABC1, an ATP binding cassette transporter required for phagocytosis of apoptotic cells, generates a regulated anion flux after expression in Xenopus laevis oocytes. J. Biol. Chem. 272: 2695-2699, 1997.
Decottignies, A.; Goffeau, A.: Complete inventory of the yeast ABC proteins. Nature Genet. 15: 137-145, 1997.
Guo, Z.; Inazu, A.; Yu, W.; Suzumura, T.; Okamoto, M.; Nohara, A.; Higashikata, T.; Sano, R.; Wakasugi, K.; Hayakawa, T.; Yoshida, K.; Suchiro, T.; Schmitz, G.; Mabuchi, H.: Double deletions and missense mutations in the first nucleotide-binding fold of the ATP-binding cassette transporter A1 (ABCA1) gene in Japanese patients with Tangier disease. J. Hum. Genet. 47: 325-329, 2002.
Hong, S. H.; Rhyne, J.; Zeller, K.; Miller, M.: Novel ABCA1 compound variant associated with HDL cholesterol deficiency. Biochim. Biophys. Acta 1587: 60-64, 2002.
Huang, W.; Moriyama, K.; Koga, T.; Hua, H.; Ageta, M.; Kawabata, S.; Mawatari, K.; Imamura, T.; Eto, T.; Kawamura, M.; Teramoto, T.; Sasaki, J.: Novel mutations in ABCA1 gene in Japanese patients with Tangier disease and familial high density lipoprotein deficiency with coronary heart disease. Biochim. Biophys. Acta 1537: 71-78, 2001.
Ishii, J.; Nagano, M.; Kujiraoka, T.; Ishihara, M.; Egashira, T.; Takada, D.; Tsuji, M.; Hattori, H.; Emi, M.: Clinical variant of Tangier disease in Japan: mutation of the ABCA1 gene in hypoalphalipoproteinemia with corneal lipidosis. J. Hum. Genet. 47: 366-369, 2002.
Jennings, M. W.; Jones, R. W.; Wood, W. G.; Weatherall, D. J.: Analysis of an inversion within the human beta globin gene cluster. Nucleic Acids Res. 13: 2897-2906, 1985.
Kulozik, A. E.; Bellan-Koch, A.; Kohne, E.; Kleihauer, E.: A deletion/inversion rearrangement of the beta-globin gene cluster in a Turkish family with delta-beta(0)-thalassemia intermedia. Blood 79:2455-2459, 1992.
Langmann, T.; Klucken, J.; Reil, M.; Liebisch, G.; Luciani, M.-F.; Chimini, G.; Kaminski, W. E.; Schmitz, G.: Molecular cloning of the human ATP-binding cassette transporter 1 (hABC1): evidence for sterol-dependent regulation in macrophages. Biochem. Biophys. Res. Commun. 257: 29-33, 1999.
Lawn, R. M.; Wade, D. P.; Garvin, M. R.; Wang, X.; Schwartz, K.; Porter, J. G.; Seilhamer, J. J.; Vaughan, A. M.; Oram, J. F.: The Tangier disease gene product ABC1 controls the cellular apolipoprotein-mediated lipid removal pathway. J. Clin. Invest. 104: R25-R31, 1999.
Luciani, M. F.; Denizot, F.; Savary, S.; Mattei, M. G.; Chimini, G.: Cloning of two novel ABC transporters mapping on human chromosome 9. Genomics 21: 150-159, 1994.
Marcil, M.; Boucher, B.; Krimbou, L.; Solymoss, B. C.; Davignon, J.; Frohlich, J.; Genest, J., Jr.: Severe familial HDL deficiency in French-Canadian kindreds: clinical, biochemical, and molecular characterization. Arterioscler. Thromb. Vasc. Biol. 15: 1015-1024, 1995.
Marcil, M.; Yu, L.; Krimbou, L.; Boucher, B.; Oram, J. F.; Cohn, J. S.; Genest, J., Jr.: Cellular cholesterol transport and effluxin fibroblasts are abnormal in subjects with familial HDL deficiency. Arterioscler. Thromb. Vasc. Biol. 19: 159-169, 1999.
Pullinger, C. R.; Hakamata, H.; Duchateau, P. N.; Eng, C.; Aouizerat, B. E.; Cho, M. H.; Fielding, C. J.; Kane, J. P.: Analysis of hABCl gene 5-prime end: additional peptide sequence, promoter region, and four polymorphisms. Biochem. Biophys. Res. Commun. 271: 451-455, 2000.
Petit, M. M. R.; Schoenmakers, E. F. P. M.; Huysmans, C.; Geurts, J. M. W.; Mandahl, N.; Van de Ven, W. J. M.: LHFP, a novel translocation partner gene of HMGIC in a lipoma, is a member of a new family of LHFP-like genes. Genomics 57: 438-441, 1999.
Lapicka-Bodzioch, K.; Bodzioch, M.; Krull, M.; Kielar, D.; Probst, M.; Kiec, B.; Andrikovics, H.; Bottcher, A.; Hubacek, J.; Aslanidis, C.; Suttorp, N.; Schmitz, G.: Homogeneous assay based on 52 primersets to scan for mutations of the ABCA1 gene and its application ingenetic analysis of a new patient with familial high-density lipoprotein deficiency syndrome. Biochim. Biophys. Acta 1537: 42-48, 2001.
McNeish, J.; Aiello, R. J.; Guyot, D.; Turi, T.; Gabel, C.; Aldinger, C.; Hoppe, K. L.; Roach, M. L.; Royer, L. J.; de Wet, J.; Broccardo, C.; Chimini, G.; Francone, O. L.: High density lipoprotein deficiency and foam cell accumulation in mice with targeted disruption of ATP-binding cassette transporter-1. Proc. Nat. Acad. Sci. 97: 4245-4250, 2000.
Nishita, Y.; Yoshida, I.; Sado, T.; Takagi, N.: Genomic imprinting and chromosomal localization of the human MEST gene. Genomics 36:539-542, 1996.
Orso, E.; Broccardo, C.; Kaminski, W. E.; Bottcher, A.; Liebisch, G.; Drobnik, W.; Gotz, A.; Chambenoit, O.; Diederich, W.; Langmann, T.; Spruss, T.; Luciani, M.-F.; Rothe, G.; Lackner, K. J.; Chimini, G.; Schmitz, G.: Transport of lipids from Golgi to plasma membrane is defective in Tangier disease patients and Abc1-deficient mice. Nature Genet. 24: 192-196, 2000.
Ahrendt, S. A.; Decker, P. A.; Alawi, E. A.; Zhu, Y.; Sanchez-Cespedes, M.; Yang, S. C.; Haasler, G. B.; Kajdacsy-Balla, A.; Demeure, M. J.; Sidransky, D.: Cigarette smoking is strongly associated with mutation of the K-ras gene in patients with primary adenocarcinoma of the lung. Cancer 92:1525-1530, 2001.
Almoguera, C.; Shibata, D.; Forrester, K.; Martin, J.; Armheim, N.; Perucho, M.: Most human carcinomas of the exocrine pancreas contain mutant c-K-ras genes. Cell 53: 549-554, 1988
Andreyev, H. J. N.; Tilsed, J. V. T.; Cunningham, D.; Sampson, S. A.; Norman, A. R.; Schneider, H. J.; Clarke, P. A.: Kras mutationsin patients with early colorectal cancers. Gut 41: 323-329, 1997.
Bollag, G.; Adler, F.; elMasry, N.; McCabe, P. C.; Connor, E., Jr.; Thompson, P.; McCormick, F.; Shannon, K.: Biochemical characterization of a novel KRAS insertion mutation from a human leukemia. J. Biol. Chem. 271: 32491-32494, 1996.
Burmer, G. C.; Loeb, L. A.: Mutations in the KRAS2 oncogene during progressive stages of human colon carcinoma. Proc. Nat. Acad. Sci. 86:2403-2407, 1989.
Capon, D. J.; Seeburg, P. H.; McGrath, J. P.; Hayflick, J. S.; Edman, U.; Levinson, A. D.; Goeddel, D. V.: Activation of Ki-ras2 gene in human colon and lung carcinomas by two different point mutations. Nature 304:507-513, 1983.
Cubilla, A. L.; Fitzgerald, P. J.: Cancer Res. 36: 2690-2698, 1976.
Der, C. J.; Cooper, G. M.: Altered gene products are associated with activation of cellular ras-k genes in human lung and colon carcinomas. Cell 32:201-208, 1983.
Feig, L. A.; Bast, R. C., Jr.; Knapp, R. C.; Cooper, G. M.: Somatic activation of ras-K gene in a human ovarian carcinoma. Science 223:698-701, 1984.
Grimmond, S. M.; Raghavan, D.; Russell, P. J.: Detection of arare point mutation in Ki-ras of a human bladder cancer xenograft by polymerase chain reaction and direct sequencing. Urol. Res. 20:121-126, 1992.
Hayashi, N.; Sugai, S.; Ito, I.; Nakamori, S.; Ogawa, M.; Nakamura, Y.: Ethnic difference in the pattern of K-ras oncogene mutations in human colorectal cancers. Hum. Mutat. 8: 258-261, 1996.
Marx, S. O.; Reiken, S.; Hisamatsu, Y.; Jayaraman, T.; Burkhoff, D.; Rosemblit, N.; Marks, A. R.: PKA phosphorylation dissociates FKBP12.6 from the calcium release channel (ryanodine receptor): defective regulation in failing hearts. Cell 101: 365-376, 2000.
Albrecht, B.; Weber, K.; Pongs, O.: Characterization of a voltage-activated K-channel gene cluster on human chromosome 12p13. Receptors Channels 3:213-220, 1995.
Grupe, A.; Schroter, K. H.; Ruppersberg, J. P.; Stocker, M.; Drewes, T.; Beckh, S.; Pongs, O.: Cloning and expression of a human voltage-gated potassium channel: a novel member of the RCK potassium channel family. EMBO J. 9: 1749-1756, 1990.
Klocke, R.; Roberds, S. L.; Tamkun, M. M.; Gronemeier, M.; Augustin, A.; Albrecht, B.; Pongs, O.; Jockusch, H.: Chromosomal mapping in the mouse of eight K(+)-channel genes representing the four Shaker-like subfamilies Shaker, Shab, Shaw, and Shal. Genomics 18: 568-574, 1993.
Kiefer, M. C.; Tucker, J. E.; Joh, R.; Landsberg, K. E.; Saltman, D.; Barr, P. J.: Identification of a second human subtilisin-like protease gene in the fes/fps region of chromosome 15. DNA Cell Biol. 10:757-769, 1991.
Orth, K.; Palmer, L. E.; Bao, Z. Q.; Stewart, S.; Rudolph, A. E.; Bliska, J. B.; Dixon, J. E.: Inhibition of the mitogen activated protein kinase kinase super family by a Yersinia effector. Science 285:1920-1923, 1999.
Rampoldi, L.; Zimbello, R.; Bortoluzzi, S.; Tiso, N.; Valle, G.; Lanfranchi, G.; Danieli, G. A.: Chromosomal localization of four MAPK signaling cascade genes: MEK1, MEK3, MEK4 and MEKK5. Cytogenet. Cell Genet. 78: 301-303, 1997.
Collu, R.; Tang, J.; Castagne, J.; Lagace, G.; Masson, N.; Huot, C.; Deal, C.; Delvin, E.; Faccenda, E.; Eidne, K. A.; Van Vliet, G.: A novel mechanism for isolated central hypothyroidism: inactivating mutations in the thyrotropin-releasing hormone receptor gene. J. Clin. Endocr. Metab. 82: 1361-1365, 1997
Katsanis, N.; Fitzgibbon, J.; Fisher, E. M. C.: Paralogy mapping: identification of a region in the human MHC triplicated onto human chromosomes 1 and 9 allows the prediction and isolation of novel PBX and NOTCH loci. Genomics 35: 101-108, 1996.
Rohen, C.; Caselitz, J.; Stern, C.; Wanschura, S.; Schoenmakers, E. F.; Van de Ven, W. J.; Barnitzke, S.; Bullerdiek, J.: A hamartoma of the breast with an aberration of 12q mapped to the MAR region by fluorescence in situ hybridization. Genes Chromosomes Cancer 84:82-84, 1995.
Schoenmakers, E. F. P. M.; Huysmans, C.; Van de Ven, W. J. M.: Allelic knockout of novel splice variants of human recombination repair gene RAD51B in t(12;14) uterine leiomyomas. Cancer Res. 59:19-23, 1999.
Zhou, X.; Benson, K. F.; Ashar, H. R.; Chada, K.: Mutation responsible for the mouse pygmy phenotype in the developmentally regulated factor HMGI-C. Nature 377: 771-774, 1995.
Holash, J.; Maisonpierre, P. C.; Compton, D.; Boland, P.; Alexander, C. R.; Zagzag, D.; Yancopoulos, G. D.; Wiegand, S. J.: Vessel cooption, regression, and growth in tumors mediated by angiopoietins and VEGF. Science 284:1994-1998, 1999
Thurston, G.; Suri, C.; Smith, K.; McClain, J.; Sato, T. N.; Yancopoulos, G. D.; McDonald, D. M.: Leakage-resistant blood vessels in mice transgenically overexpressing angiopoietin-1. Science 286: 2511-2514, 1999.
Rampoldi, L.; Dobson-Stone, C.; Rubio, J. P.; Danek, A.; Chalmers, R. M.; Wood, N. W.; Verellen, C.; Ferrer, X.; Malandrini, A.; Fabrizi, G. M.; Brown, R.; Vance, J.; Pericak-Vance, M.; Rudolf, G.; Carre, S.; Alonso, E.; Manfredi, M.; Nemeth, A. H.; Monaco, A. P.: A conserved sorting-associated protein is mutant in chorea-acanthocytosis. Nature Genet. 28: 119-120, 2001.
Wong-Staal, F.; Dalla-Favera, R.; Franchini, G.; Gelmann, E. P.; Gallo, R. C.: Three distinct genes in human DNA related to the transforming genes of mammalian sarcoma retroviruses. Science 213: 226-228, 1981.
Aurias, A.; Rimbaut, C.; Buffe, D.; Dubousset, J.; Mazabraud, A.: Chromosomal translocations in Ewing's sarcoma. (Letter) New Eng. J. Med. 309: 496-497, 1983.
Bartram, C. R.; de Klein, A.; Hagemeijer, A.; Grosveld, G.; Heisterkamp, N.; Groffen, J.: Localization of the human c-sis oncogene in Ph-1-positive and Ph-1-negative chronic myelocytic leukemia by in situ hybridization. Blood 63:223-225, 1984
Bechet, J.-M.; Bornkamm, G.; Freese, U.-K.; Lenoir, G. M.: The c-sis oncogene is not activated in Ewing's sarcoma. (Letter) New Eng. J. Med. 310: 393 only, 1984.
Bishop, J. M.: Enemies within: the genesis of retrovirus oncogenes. Cell 23:5-6, 1981.
Bolger, G. B.; Stamberg, J.; Kirsch, I. R.; Hollis, G. F.; Schwarz, D. F.; Thomas, G. H.: Chromosomal translocation t(14;22) and oncogene(c-sis) variant in a pedigree with familial meningioma. New Eng. J. Med. 312: 564-567, 1985.
Cohen, J. B.; Levinson, A. D.: A point mutation in the last intron responsible for increased expression and transforming activity of the c-Ha-ras oncogene. Nature 334: 119-124, 1988.
Collins, T.; Ginsburg, D.; Boss, J. M.; Orkin, S. H.; Pober, J. S.: Cultured human endothelial cells express platelet-derived growth factor B chain: cDNA cloning and structural analysis. Nature 316:748-750, 1985.
Dalla-Favera, R.; Gallo, R. C.; Giallongo, A.; Croce, C.: Chromosomal localization of the human homolog (c-sis) of the simian sarcoma virusonc gene. Science 218: 686-688, 1982.
Dalla-Favera, R.; Gelmann, E. P.; Gallo, R. C.; Wong-Staal, F.: A human one gene homologous to the transforming gene (v-sis) of simian sarcoma virus. Nature 292: 31-35, 1981.
Deuel, T. F.; Huang, J. S.; Huang, S. S.; Stroobant, P.; Waterfield, M. D.: Expression of a platelet-derived growth factor-like protein in simian sarcoma virus transformed cells. Science 221: 1348-1350, 1983.
Devare, S. G.; Reddy, E. P.; Law, J. D.; Robbins, K. C.; Aaronson, S. A.: Nucleotide sequence of the simian sarcoma virus genome: demonstration that its acquired cellular sequences encode the transforming gene product p28-sis. Proc. Nat. Acad. Sci. 80: 731-735, 1983.
Benson, K. F.; Horwitz, M.; Wolff, J.; Friend, K.; Thompson, E.; White, S.; Richards, R. I.; Raskind, W. H.; Bird, T. D.: CAG repeat expansion in autosomal dominant familial spastic paraparesis: novel expansion in a subset of patients. Hum. Molec. Genet. 7: 1779-1786, 1998.
Fonknechten, N.; Mavel, D.; Byrne, P.; Davoine, C.-S.; Cruaud, C.; Boentsch, D.; Samson, D.; Coutinho, P.; Hutchinson, M.; McMonagle, P.; Burgunder, J.-M.; Tartaglione, A.; and 10 others: Spectrum of SPG4 mutations in autosomal dominant spastic paraplegia. Hum. Molec. Genet. 9: 637-644, 2000
Nielsen, J. E.; Koefoed, P.; Abell, K.; Hasholt, L.; Eiberg, H.; Fenger, K.; Niebuhr, E.; Sorensen, S. A.: CAG repeat expansion in autosomal dominant pure spastic paraplegia linked to chromosome 2p21-p24. Hum. Molec. Genet. 6: 1811-1816, 1997.
Svenson, 1. K.; Ashley-Koch, A. E.; Gaskell, P. C.; Riney, T. J.; Cumming, W. J. K.; Kingston, H. M.; Hogan, E. L.; Boustany, R.-M. N.; Vance, J. M.; Nance, M. A.; Pericak-Vance, M. A.; Marchuk, D. A.: Identification and expression analysis of spastin gene mutations in hereditary spastic paraplegia. Am. J. Hum. Genet. 68: 1077-1085, 2001.
Asch, A. S.; Barnwell, J.; Silverstein, R. L.; Nachman, R. L.: Isolation of the thrombospondin membrane receptor. J. Clin. Invest. 79:1054-1061, 1987.
de Fraipont, F.; E1 Atifi, M.; Gicquel, C.; Bertagna, X.; Chambaz, E. M.; Feige, J. J.: Expression of the angiogenesis markers vascular endothelial growth factor-A, thrombospondin-1, and platelet-derived endothelial cell growth factor in human sporadic adrenocortical tumors: correlation with genotypic alterations. J. Clin. Endocr. Metab. 85:4734-4741, 2000.
Dixit, V. M.; Hennessy, S. W.; Grant, G. A.; Rotwein, P.; Frazier, W. A.: Characterization of a cDNA encoding the heparin and collagen binding domains of human thrombospondin. Proc. Nat. Acad. Sci. 83:5449-5453, 1986.
Frazier, W. A.: Thrombospondin: a modular adhesive glyco protein of platelets and nucleated cells. J. Cell Biol. 105: 625-632, 1987.
Jaffe, E.; Bornstein, P.; Disteche, C. M.: Mapping of the thrombospondin gene to human chromosome 15 and mouse chromosome 2 by in situ hybridization. Genomics 7:123-126, 1990.
Lawler, J.; Sunday, M.; Thibert, V.; Duquette, M.; George, E. L.; Rayburn, H.; Hynes, R. O.: Thrombospondin-1 is required for normal murine pulmonary homeostasis and its absence causes pneumonia. J. Clin. Invest. 101: 982-992, 1998.
Rodriguez-Manzaneque, J. C.; Lane, T. F.; Ortega, M. A.; Hynes, R. O.; Lawler, J.; Iruela-Arispe, M. L.: Thrombospondin-1 suppresses spontaneous tumor growth and inhibits activation of matrix metalloproteinase-9 and mobilization of vascular endothelial growth factor. Proc. Nat. Acad. Sci. 98: 12485-12490, 2001.
Wolf, F. W.; Eddy, R. L.; Shows, T. B.; Dixit, V. M.: Structure and chromosomal localization of the human thrombospondin gene. Genomics 6:685-691, 1990
Peter, D.; Finn, J. P.; Klisak, I.; Liu, Y.; Kojis, T.; Heinzmann, C.; Roghani, A.; Sparkes, R. S.; Edwards, R. H.: Chromosomal localization of the human vesicular amine transporter genes. Genomics 18: 720-723, 1993.
Roghani, A.; Welch, C.; Xia, Y.-R.; Liu, Y.; Peter, D.; Finn, J. P.; Edwards, R. H.; Lusis, A. J.: Assignment of the mouse vesicular monoamine transporter genes, Slc18a1 and Slc18a2, to chromosomes 8 and 19 by linkage analysis. Mammalian Genome 7: 393-394, 1996.
Forsell, P. K. A. L.; Boie, Y.; Montalibet, J.; Collins, S.; Kennedy, B. P.: Genomic characterization of the human and mouse protein tyrosine phosphatase-1B genes. Gene 260: 145-153, 2000.
Gu, H. F.; Almgren, P.; Lindholm, E.; Frittitta, L.; Pizzuti, A.; Trischitta, V.; Groop, L. C.: Association between the human glycoprotein PC-1 gene and elevated glucose and insulin levels in paired-sibling analysis. Diabetes 49: 1601-1603, 2000.
Haj, F. G.; Verveer, P. J.; Squire, A.; Neel, B. G.; Bastiaens, P. I. H.: Imaging sites of receptor dephosphorylation by PTP1B on the surface of the endoplasmic reticulum. Science 295: 1708-1711, 2002.
Jia, Z.; Barford, D.; Flint, A. J.; Tonks, N. K.: Structural basis for phosphotyrosine peptide recognition by protein tyrosine phosphatase 1B. Science 268: 1754-1758, 1995.
Kennedy, B. P.; Ramachandran, C.: Protein tyrosine phosphatase-1B in diabetes. Biochem. Pharm. 60: 877-883, 2000.
Elchebly, M.; Payette, P.; Michaliszyn, E.; Cromlish, W.; Collins, S.; Loy, A. L.; Normandin, D.; Cheng, A.; Himms-Hagen, J.; Chan, C.-C.; Ramachandran, C.; Gresser, M. J.; Tremblay, M. L.; Kennedy, B. P.: Increased insulin sensitivity and obesity resistance in mice lacking the protein tyrosine phosphatase-1B gene. Science 283: 1544-1548, 1999.
Mok, A.; Cao, H.; Zinman, B.; Hanley, A. J. G.; Harris, S. B.; Kennedy, B. P.; Hegele, R. A.: A single nucleotide polymorphism in protein tyrosine phosphatase PTP-1B is associated with protection from diabetes or impaired glucose tolerance in Oji-Cree. J. Clin. Endocr. Metab. 87: 724-727, 2002.
Tonks, N. K.; Diltz, C. D.; Fischer, E. H.: Purification of the major protein-tyrosine-phosphatases of human placenta. J. Biol. Chem. 263:6722-6730, 1988.
Chan, J. Y.; Cheung, M.-C.; Moi, P.; Chan, K.; Kan, Y. W.: Chromosomal localization of the human NF-E2 family of bZIP transcription factors by fluorescence in situ hybridization. Hum. Genet. 95: 265-269, 1995.
Chan, J. Y.; Han, X.-L.; Kan, Y. W.: Cloning of Nrf1, an NFE2-related transcription factor, by genetic selection in yeast. Proc. Nat. Acad. Sci. 90: 11371-11375, 1993.
Chan, J. Y.; Kwong, M.; Lu, R.; Chang, J.; Wang, B.; Yen, T. S. B.; Kan, Y. W.: Targeted disruption of the ubiquitous CNC-bZIP transcription factor, Nrf-1, results in anemia and embryonic lethality in mice. EMBO J. 17: 1779-1787, 1998.
Luna, L.; Johnsen, O.; Skartlien, A.; Pedeutour, F.; Turc-Carel, C.; Prydz, H.; Kolsto, A.-B.: Molecular cloning of a putative novel human bZIP transcription factor on chromosome 17q22. Genomics 22:553-562, 1994.
Luna, L.; Johnsen, O.; Skartlien, A. H.; Pedeutour, F.; Turc-Carel, C.; Prydz, H.; Kolsto, A.-B.: Molecular cloning of a putative novel human bZIP transcription factor on chromosome 17q22. Genomics 22:553-562, 1994.
McKie, J.; Johnstone, K.; Mattei, M.-G.; Scambler, P.: Cloning and mapping of murine Nfe2I1. Genomics 25: 716-719, 1995.
McKie, J.; Scambler, P. J.: The Nfe2I1 gene maps to distal mouse chromosome 11. Mammalian Genome 7: 89-90, 1996.
Liu, P.; Wakamiya, M.; Shea, M. J.; Albrecht, U.; Behringer, R. R.; Bradley, A.: Requirement for Wnt3 in vertebrate axis formation. Nature Genet. 22: 361-365, 1999.
Rider, S. H.; Gorman, P. A.; Shipley, J.; Roeling, H.; Nusse, R.; Xu, W.; Sheer, D.; Solomon, E.: Localisation of the human int-4 (INT4) gene. (Abstract) Cytogenet. Cell Genet. 51: 1066 only, 1989.
Roelink, H.; Wang, J.; Black, D. M.; Solomon, E.; Nusse, R.: Molecular cloning and chromosomal localization to 17q21 of the human WNT3 gene. Genomics 17:790-792, 1993.
Lossie, A. C.; Gordon, D. F.; Camper, S. A.: Localization of thyrotropin-releasing hormone receptor and thyrotroph embryonic factor on mouse chromosome 15. Mammalian Genome 4: 621-623, 1993.
Matre, V.; Karlsen, H. E.; Wright, M. S.; Lundell, I.; Fjeldheim, A. K.; Gabrielsen, O. S.; Larhammar, D.; Gautvik, K. M.: Molecular cloning of a functional human thyrotropin-releasing hormone receptor. Biochem. Biophys. Res. Commun. 195: 179-185, 1993.
Morrison, N.; Duthie, S. M.; Boyd, E.; Eidne, K. A.; Connor, J. M.: Assignment of the gene encoding the human thyrotropin-releasing hormone receptor to 8q23 by fluorescence in situ hybridization. Hum. Genet. 93: 716-718, 1994.
Straub, R. E.; Frech, G. C.; Joho, R. H.; Gershengorn, M. C.: Expression cloning of a cDNA encoding the mouse pituitary thyrotropin-releasing hormone receptor. Proc. Nat. Acad. Sci. 87: 9514-9518, 1990.
Yamada, M.; Monden, T.; Konaka, S.; Mori, M.: Assignment of human thyrotropin-releasing hormone (TRH) receptor gene to chromosome 8. Somat. Cell Molec. Genet. 19: 577-580, 1993.
Zhao, D.; Yang, J.; Jones, K. E.; Gerald, C.; Suzuki, Y.; Hogan, P. G.; Chin, W. W.; Tashjian, A. H., Jr.: Molecular cloning of a complementary deoxyribonucleic acid encoding the thyrotropin-releasing hormone receptor and regulation of its messenger ribonucleic acid in rat GH cells. Endocrinology 130: 3529-3536, 1992.
Brennan, T. J.; Seeley, W. W.; Kilgard, M.; Schreiner, C. E.; Tecott, L. H.: Sound-induced seizures in serotonin 5-HT-2C receptor mutant mice. Nature Genet. 16: 387-390, 1997.
Gurevich, I.; Tamir, H.; Arango, V.; Dwork, A. J.; Mann, J. J.; Schmauss, C.: Altered editing of serotonin 2C receptor pre-mRNA in the prefrontal cortex of depressed suicide victims. Neuron 34: 349-356, 2002.
Hall, C. S.: Genetic differences in fatal audiogenic seizures: between two inbred strains of house mice. J. Hered. 38: 3-6, 1947.
Lappalainen, J.; Zhang, L.; Dean, M.; Oz, M.; Ozaki, N.; Yu, D.; Virkkunen, M.; Weight, F.; Linnoila, M.; Goldman, D.: Identification, expression, and pharmacology of a cys(23)-ser(23) substitution in the human 5-HT(2C) receptor gene (HTR2C). Genomics 27: 274-279, 1995.
Milatovich, A.; Hsieh, C.-L.; Bonaminio, G.; Tecott, L.; Julius, D.; Francke, U.: Serotonin receptor 1c gene assigned to X chromosome in human (band q24) and mouse (bands D-F4). Hum. Molec. Genet. 1:681-684, 1992.
Tecott, L. H.; Sun, L. M.; Akana, S. F.; Strack, A. M.; Lowenstein, D. H.; Dallman, M. F.; Julius, D.: Eating disorder and epilepsy in mice lacking 5-HT2C serotonin receptors. Nature 374: 542-546, 1995.
Prakash, S. K.; Paylor, R.; Jenna, S.; Lamarche-Vane, N.; Armstrong, D. L.; Xu, B.; Mancini, M. A.; Zoghbi, H. Y.: Functional analysis of ARHGAP6, a novel GTPase-activating protein for RhoA. Hum. Molec. Genet. 9: 477-488, 2000.
Schaefer, L.; Prakash, S.; Zoghbi, H. Y.: Cloning and characterization of a novel rho-type GTPase-activating protein gene (ARHGAP6) from the critical region for microphthalmia with linear skin defects. Genomics 46:268-277, 1997.
Aman, M. J.; Tayebi, N.; Obiri, N. I.; Puri, R. K.; Modi, W. S.; Leonard, W. J.: cDNA cloning and characterization of the human interleukin 13 receptor alpha chain. J. Biol. Chem. 271: 29265-29270, 1996.
Guo, J.; Apiou, F.; Mellerin, M.-P.; Lebeau, B.; Jacques, Y.; Minvielle, S.: Chromosome mapping and expression of the human interleukin-13 receptor. Genomics 42: 141-5, 1997.
Hilton, D. J.; Zhang, J.-G.; Metcalf, D.; Alexander, W. S.; Nicola, N. A.; Willson, T. A.: Cloning and characterization of a binding subunit of the interleukin 13 receptor that is also a component of the interleukin 4 receptor. Proc. Nat. Acad. Sci. 93: 497-501, 1996.
Kleijnen, M. F.; Shih, A. H.; Zhou, P.; Kumar, S.; Soccio, R. E.; Kedersha, N. L.; Gill, G.; Howley, P. M.: The hPLIC proteins may provide a link between the ubiquitination machinery and the proteasome. Molec. Cell 6: 409-419, 2000.
Riesewijk, A. M.; Blagitko, N.; Schinzel, A. A.; Hu, L.; Schulz, U.; Hamel, B. C. J.; Ropers, H.-H.; Kalscheuer, V. M.: Evidence against a major role of PEG1/MEST Silver-Russell syndrome. Europ. J. Hum. Genet. 6: 114-120, 1998.
Riesewijk, A. M.; Hu, L.; Schulz, U.; Tariverdian, G.; Hoglund, P.; Kere, J.; Ropers, H.-H.; Kalscheuer, V. M.: Monoallelic expression of human PEG1/MEST is paralleled by parent-specific methylation infetuses. Genomics 42: 236-244, 1997.
Sado, T.; Nakajima, N.; Tada, M.; Takagi, N.: A novel mesoderm-specific cDNA isolated from a mouse embryonal carcinoma cell line. Dev. Growth Differ. 35: 551-560, 1993.
Jayakumar, A.; Chirala, S. S.; Chinault, A. C.; Baldini, A.; Abu-Elheiga, L.; Wakil, S. J.: Isolation and chromosomal mapping of genomic clones encoding the human fatty acid synthase gene. Genomics 23: 420-424, 1994.
Jayakumar, A.; Tai, M.-H.; Huang, W.-Y.; A1-Feel, W.; Hsu, M.; Abu-Elheiga, L.; Chirala, S. S.; Wakil, S. J.: Human fatty acid synthase: properties and molecular cloning. Proc. Nat. Acad. Sci. 92: 8695-8699, 1995.
Loftus, T. M.; Jaworsky, D. E.; Frehywot, G. L.; Townsend, C. A.; Ronnett, G. V.; Lane, M. D.; Kuhajda, F. P.: Reduced food intake and body weight in mice treated with fatty acid synthase inhibitors. Science 288:2379-2381, 2000.
Wakil, S. J.: Fatty acid synthase, a proficient multifunctional enzyme. Biochemistry 28: 4523-4530, 1989.
Ye, Q.; Chung, L. W. K.; Li, S.; Zhau, H. E.: Identification of a novel FAS/ER-alpha fusion transcript expressed in human cancer cells. Biochim. Biophys. Acta 1493: 373-377, 2000.
Hansen, J. J.; Durr, A.; Cournu-Rebeix, I.; Georgopoulos, C.; Ang, D.; Nielsen, M. N.; Davoine, C.-S.; Brice, A.; Fontaine, B.; Gregersen, N.; Bross, P.: Hereditary spastic paraplegia SPG13 is associated with a mutation in the gene encoding the mitochondrial chaperonin Hsp60. Am. J. Hum. Genet. 70: 1328-1332, 2002.
Todd, M. J.; Viitanen, P. V.; Lorimer, G. H.: Dynamics of the chaperonin ATPase cycle: implications for facilitated protein folding. Science 265:659-666, 1994.
Di Paola, R.; Frittitta, L.; Miscio, G.; Bozzali, M.; Baratta, R.; Centra, M.; Spampinato, D.; Santagati, M. G.; Ercolino, T.; Cisternino, C.; Soccio, T. Mastroianno, S.; Tassi, V.; Almgren, P.; Pizzuti, A.; Vigneri, R.; Trischitta, V.: A variation in 3-prime UTR of hPTP1B increases specific gene expression and associates with insulin resistance. Am. J. Hum. Genet. 70: 806-812, 2002
Eidne, K.; Taylor, P.; Connor, M.; Duthie, S.: Isolation, characterization and chromosomal localization of the human thyrotropin releasing hormone receptor. (Abstract) 75th Annual Meeting of the Endocr. Soc. 437 only, 1993.
Gedde-Dahl, T., Jr.; Olaisen, B.; Teisberg, P.; Wilhelmy, M. C.; Mevag, B.; Helland, R.: The locus for apolipoprotein E (apoE) is close to the Lutheran (Lu) blood group locus on chromosome 19. Hum. Genet. 67: 178-182, 1984.
Ogasawara, M.; Kim, S.-C.; Adamik, R.; Togawa, A.; Ferrans, V. J.; Takeda, K.; Kirby, M.; Moss, J.; Vaughan, M.: Similarities in function and gene structure of cytohesin-4 and cytohesin-1, guanine nucleotide-exchange proteins for ADP-ribosylation factors. J. Biol. Chem. 275: 3221-3230, 2000.
Helwig, U.; Imai, K.; Schmahl, W.; Thomas, B. E.; Varnum, D. S.; Nadeau, J. H.; Balling, R.: Interaction between undulated and Patch leads to an extreme form of spina bifida in double-mutant mice. Nature Genet. 11: 60-63, 1995.
Barnett, T.; Pick1e, W., II; Rae, P. M. M.; Hart, J.; Kamarck, M.; Elting, J.: Pregnancy-specific beta-1-glycoproteins are related to carcinoembryonic antigens and map to chromosome 19. (Abstract) Cytogenet. Cell Genet. 51: 958, 1989
Barnett, T. R.; Pick1e, W., II; Rae, P. M. M.; Hart, J.; Kamarck, M.; Elting, J.: Human pregnancy-specific beta(1)-glycoproteins are coded within chromosome 19. Am. J. Hum. Genet. 44: 890-893, 1989.
Bartels, I.; Lindemann, A.: Maternal levels of pregnancy-specific beta-1-glyco protein (SP-1) are elevated in pregnancies affected by Down's syndrome. Hum. Genet. 80: 46-48, 1988.
Brandriff, B. F.; Gordon, L. A.; Tynan, K. T.; Olsen, A. S.; Mohrenweiser, H. W.; Fertitta, A.; Carrano, A. V.; Trask, B. J.: Order and genomic distances among members of the carcinoembryonic antigen (CEA) gene family determined by fluorescence in situ hybridization. Genomics 12:773-779, 1992.
Chan, W.-Y.; Qiu, W.-R.: Human pregnancy-specific beta-1 glycoprotein is encoded by multiple genes localized on two chromosomes. Am. J. Hum. Genet. 43: 152-159, 1988.
Khan, W. N.; Teglund, S.; Bremer, K.; Hammarstrom, S.: The pregnancy-specific glyco protein family of the immunoglobulin super family: identification of new members and estimation of family size. Genomics 12: 780-787, 1992
Niemann, S. C.; Flake, A.; Bohn, H.; Bartels, I.: Pregnancy-specific beta-1-glyco protein: cDNA cloning, tissue expression, and species specificity of one member of the PSBG family. Hum. Genet. 82: 239-243, 1989.
Niemann, S. C.; Schonk, D.; van Dijk, P.; Wieringa, B.; Grzeschik, K.-H.; Bartels, I.: Regional localization of the gene encoding pregnancy specific beta-1-glyco protein 1 (PSBG1) to human chromosome 19q13.1. Cytogenet. Cell Genet. 52: 95-97, 1989.
Niemann, S. C.; Schonk, D.; van Dijk, P. E.; Grzeschik, K.-H.; Bartels, I.: Chromosomal assignment of a cDNA clone encoding pregnancy-specific beta-1-glyco protein to chromosome 19. (Abstract) Cytogenet. Cell Genet. 51: 1053, 1989.
Olsen, A.; Teglund, S.; Nelson, D.; Gordon, L.; Copeland, A.; Georgescu, A.; Carrano, A.; Hammarstrom, S.: Gene organization of the pregnancy-specific glyco protein region on human chromosome 19: assembly and analysis of a 700-kb cosmid contig spanning the region. Genomics 23:659-668, 1994.
Streydio, C.; Swillens, S.; Georges, M.; Szpirer, C.; Vassart, G.: Structure, evolution and chromosomal localization of the human pregnancy-specific beta-1 glyco protein gene family. Genomics 6:579-592, 1990. Note: Erratum: Genomics 7: 661-662, 1990.
Teglund, S.; Olsen, A.; Khan, W. N.; Frangsmyr, L.; Hammarstrom, S.: The pregnancy-specific glyco protein (PSG) gene cluster on human chromosome 19: fine structure of the 11 PSG genes and identification of 6 new genes forming a third subgroup within the carcinoembryonic antigen (CEA) family. Genomics 23: 669-684, 1994.
Thompson, J.; Koumari, R.; Wagner, K.; Barnert, S.; Schleussner, C.; Schrewe, H.; Zimmermann, W.; Muller, G.; Schempp, W.; Zaninetta, D.; Ammaturo, D.; Hardman, N.: The human pregnancy-specific glycoprotein genes are tightly linked on the long arm of chromosome 19 and are-coordinately expressed. Biochem. Biophys. Res. Commun. 167: 848-859, 1990.
Watanabe, S.; Chou, J. Y.: Isolation and characterization of complementary DNAs encoding human pregnancy-specific beta-1-glyco protein. J. Biol. Chem. 263: 2049-2054, 1988.
Olsen, A.; Teglund, S.; Nelson, D.; Gordon, L.; Copeland, A.; Georgescu, A.; Carrano, A.; Hammarstrom, S.: Gene organization of the pregnancy-specific glyco protein region on human chromosome 19: assembly and analysis of a 700-kb cosmid contig spanning the region. Genomics 23: 659-668, 1994.
Thompson, J.; Zimmermann, W.; Osthus-Bugat, P.; Schleussner, C.; Eades-Perner, A.-M.; Barnert, S.; von Kleist, S.; Willcocks, T.; Craig, I.; Tynan, K.; Olsen, A.; Mohrenweiser, H.: Long-range chromosomal mapping of the carcinoembryonic antigen (CEA) gene family cluster. Genomics 12:761-772, 1992.
Tynan, K.; Olsen, A.; Trask, B.; de Jong, P.; Thompson, J.; Zimmermann, W.; Carrano, A.; Mohrenweiser, H.: Assembly and analysis of cosmid contigs in the CEA-gene family region of human chromosome 19. Nucleic Acids Res. 20: 1629-1636, 1992.
Nobukuni, Y.; Watanabe, A.; Takeda, K.; Skarka, H.; Tachibana, M.: Analyses of loss-of-function mutations of the MITF gene suggest that haploinsufficiency is a cause of Waardenburg syndrome type 2A. Am. J. Hum. Genet. 59: 76-83, 1996.
Steingrimsson, E.; Moore, K. J.; Lamoreux, M. L.; FerreD'Amare, A. R.; Burley, S. K.; Sanders Zimring, D. C.; Skow, L. C.; Hodgkinson, C. A.; Arnheiter, H.; Copeland, N. G.; Jenkins, N. A.: Molecular basis of mouse microphthalmia (mi) mutations helps explain their developmental and phenotypic consequences. Nature Genet. 8: 256-263, 1994.
Steingrimsson, E.; Tessarollo, L.; Pathak, B.; Hou, L.; Arnheiter, H.; Copeland, N. G.; Jenkins, N. A.: Mitf and Tfe3, two members of the Mitf-Tfe family of bHLH-Zip transcription factors, have important but functionally redundant roles in osteoclast development. Proc. Nat. Acad. Sci. 99: 4477-4482, 2002.
Tachibana, M.; Perez-Jurado, L. A.; Nakayama, A.; Hodgkinson, C. A.; Li, X.; Schneider, M.; Miki, T.; Fex, J.; Francke, U.; Arnheiter, H.: Cloning of MITF, the human homolog of the mouse microphthalmia gene and assignment to chromosome 3p14.1-p12.3. Hum. Molec. Genet. 3:553-557, 1994.
Tachibana, M.; Takeda, K.; Nobukuni, Y.; Urabe, K.; Long, J. E.; Meyers, K. A.; Aaronson, S. A.; Miki, T.: Ectopic expression of MITF, a gene for Waardenburg syndrome type 2, converts fibroblasts to cells with melanocytes characteristics. Nature Genet. 14: 50-54, 1996.
Takeda, K.; Takemoto, C.; Kobayashi, I.; Watanabe, A.; Nobukuni, Y.; Fisher, D. E.; Tachibana, M.: Ser298 of MITF, a mutation site in Waardenburg syndrome type 2, is a phosphorylation site with functional significance. Hum. Molec. Genet. 9: 125-132, 2000
Tassabehji, M.; Newton, V. E.; Read, A. P.: Waardenburg syndrome type 2 caused by mutations in the human microphthalmia (MITF) gene. Nature Genet. 8: 251-255, 1994.
Watanabe, A.; Takeda, K.; Ploplis, B.; Tachibana, M.: Epistatic relationship between Waardenburg syndrome genes MITF and PAX3. Nature Genet. 18: 283-286, 1998.
Yoshitake, H.; Rittling, S. R.; Denhardt, D. T.; Noda, M.: Osteopontin-deficient mice are resistant to ovariectomy-induced bone resorption. Proc. Nat. Acad. Sci. 96: 8156-8160, 1999.
St-Onge, L.; Sosa-Pineda, B.; Chowdhury, K.; Mansouri, A.; Gruss, P.: Pax6 is required for differentiation of glucagon-producing alpha-cells in mouse pancreas. Nature 387: 406-409, 1997.
Bongarzone, I.; Vigano, E.; Alberti, L.; Borrello, M. G.; Pasini, B.; Greco, A.; Mondellini, P.; Smith, D. P.; Ponder, B. A. J.; Romeo, G.; Pierotti, M. A.: Full activation of MEN2B mutant RET by an additional MEN2A mutation or by ligand GDNF stimulation. Oncogene 16: 2295-2301, 1998.
Carlson, K. M.; Bracamontes, J.; Jackson, C. E.; Clark, R.; Lacroix, A.; Wells, S. A., Jr.; Goodfellow, P. J.: Parent-of-origin effects in multiple endocrine neoplasia type 2B. Am. J. Hum. Genet. 55:1076-1082, 1994.
Carlson, K. M.; Dou, S.; Chi, D.; Scavarda, N.; Toshima, K.; Jackson, C. E.; Wells, S. A., Jr.; Goodfellow, P. J.; Donis-Keller, H.: Single missense mutation in the tyrosine kinase catalytic domain of the RET protooncogene is associated with multiple endocrine neoplasia type 2B. Proc. Nat. Acad. Sci. 91: 1579-1583, 1994.
Mulligan, L. M.; Eng, C.; Healey, C. S.; Clayton, D.; Kwok, J. B. J.; Gardner, E.; Ponder, M. A.; Frilling, A.; Jackson, C. E.; Lehnert, H.; Neumann, H. P. H.; Thibodeau, S. N.; Ponder, B. A. J.: Specific mutations of the RET proto-oncogene are related to disease phenotype in MEN 2A and FMTC. Nature Genet. 6: 70-74, 1994.
Disteche, C. M.; Brannan, C. I.; Larsen, A.; Adler, D. A.; Schorderet, D. F.; Gearing, D.; Copeland, N. G.; Jenkins, N. A.; Park, L. S.: The human pseudoautosomal GM-CSF receptor alpha subunit gene is autosomal in mouse. Nature Genet. 1: 333-336, 1992.
Fujii, J.; Zarain-Herzberg, A.; Willard, H. F.; Tada, M.; MacLennan, D. H.: Structure of the rabbit phospholamban gene, cloning of the human cDNA, and assignment of the gene to human chromosome 6. J. Biol. Chem. 266: 11669-11675, 1991
McTiernan, C. F.; Frye, C. S.; Lemster, B. H.; Kinder, E. A.; Ogletree-Hughes, M. L.; Moravec, C. S.; Feldman, A. M.: The human phospholamban gene: structure and expression. J. Molec. Cell Cardiol. 31: 679-692, 1999.
Otsu, K.; Fujii, J.; Periasamy, M.; Difilippantonio, M.; Uppender, M.; Ward, D. C.; MacLennan, D. H.: Chromosome mapping of five human cardiac and skeletal muscle sarcoplasmic reticulum protein genes. Genomics 17:507-509, 1993.
Coleman, R. A.; Smith, W. L.; Narumiya, S.: VIII. Internationalunion of pharmacology classification of prostanoid receptors: properties, distribution, and structure of the receptors and their subtypes. Pharm. Rev. 46: 205-229, 1994.
Duncan, A. M. V.; Anderson, L. L.; Funk, C. D.; Abramovitz, M.; Adam, M.: Chromosomal localization of the human prostanoid receptor gene family. Genomics 25: 740-742, 1995.
Taketo, M.; Rochelle, J. M.; Sugimoto, Y.; Namba, T.; Honda, A.; Negishi, M.; Ichikawa, A.; Narumiya, S.; Seldin, M. F.: Mapping of the genes encoding mouse thromboxane A2 receptor and prostaglandinE receptor subtypes EP2 and EP3. Genomics 19: 585-588, 1994
Shimomura, I.; Matsuda, M.; Hammer, R. E.; Bashmakov, Y.; Brown, M. S.; Goldstein, J. L.: Decreased IRS-2 and increased SREBP-1c lead to mixed insulin resistance and sensitivity in livers of lipodystrophic and ob/ob mice. Molec. Cell 6: 77-86, 2000.
Muyan, M.; Furuhashi, M.; Sugahara, T.; Boime, I.: The carboxy-terminal region of the beta-subunits of luteinizing hormone and chorionic gonadotropin differentially influence secretion and assembly of the heterodimers. Molec. Endocr. 10: 1678-1687, 1996.
Adam, M.; Boie, Y.; Rushmore, T. H.; Muller, G.; Bastien, L.; McKee, K. T.; Metters, K. M.; Abramovitz, M.: Cloning and expression of three isoforms of the human EP(3) prostanoid receptor. FEBS Lett. 338:170-174, 1994.
Kotani, M.; Tanaka, I.; Ogawa, Y.; Suganami, T.; Matsumoto, T.; Muro, S.; Yamamoto, Y.; Sugawara, A.; Yoshimasa, Y.; Sagawa, N.; Narumiya, S.; Nakao, K.: Multiple signal transduction pathways through two prostaglandin E receptor EP3 subtype isoforms expressed in human uterus. J. Clin. Endocr. Metab. 85: 4315-4322, 2000.
Kotani, M.; Tanaka, I.; Ogawa, Y.; Usui, T.; Tamura, N.; Mori, K.; Narumiya, S.; Yoshimi, T.; Nakao, K.: Structural organization of the human prostaglandin EP(3) receptor subtype gene (PTGER3). Genomics 40:425-434, 1997.
Kotani, M.; Tanaka, I.; Ogawa, Y.; Usui, T.; Mori, K.; Ichikawa, A.; Narumiya, S.; Yoshimi, T.; Nakao, K.: Molecular cloning and expression of multiple isoforms of human prostaglandin E receptor EP(3) subtype generated by alternative messenger RNA splicing: multiple second messenger systems and tissue-specific distributions. Molec. Pharm. 48: 869-879, 1995.
Schmid, A.; Thierauch, K.-H.; Schleuning, W.-D.; Dinter, H.: Splice variants of the human EP(3) receptor for prostaglandin E(2). Europ. J. Biochem. 228: 23-30, 1995.
Ushikubi, F.; Segi, E.; Sugimoto, Y.; Murata, T.; Matsuoka, T.; Kobayashi, T.; Hizaki, H.; Tuboi, K.; Katsuyama, M.; Ichikawa, A.; Tanaka, T.; Yoshida, N.; Narumiya, S.: Impaired febrile response in mice lacking the prostaglandin E receptor subtype EP(3). Nature 395:281-284, 1998.
Kaplan, R.; Morse, B.; Huebner, K.; Croce, C.; Howk, R.; Ravera, M.; Ricca, G.; Jaye, M.; Schlessinger, J.: Cloning of three human tyrosine phosphatases reveals a multi gene family of receptor-linked protein-tyrosine-phosphatases expressed in brain. Proc. Nat. Acad. Sci. 87: 7000-7004, 1990.
Jirik, F. R.; Anderson, L. L.; Duncan, A. M. V.: The human protein-tyrosine phosphatase PTP-alpha/LRP gene (PTPA) is assigned to chromosome 20p13. Cytogenet. Cell Genet. 60: 117-118, 1992.
Jirik, F. R.; Janzen, N. M.; Melhado, I. G.; Harder, K. W.: Cloning and chromosomal assignment of a widely expressed human receptor-like protein-tyrosine phosphatase. FEBS Lett. 273: 239-242, 1990.
Matthews, R. J.; Cahir, E. D.; Thomas, M. L.: Identification of an additional member of the protein-tyrosine-phosphatase family: evidence for alternative splicing in the tyrosine phosphatase domain. Proc. Nat. Acad. Sci. 87: 4444-4448, 1990.
Rao, V. V. N. G.; Loffler, C.; Sap, J.; Schlessinger, J.; Hansmann, I.: The gene for receptor-linked protein-tyrosine-phosphatase (PTPA) is assigned to human chromosome 20p12-pter by in situ hybridization (ISH and FISH). Genomics 13: 906-907, 1992.
Schnittger, S.; Rao, V. V. N. G.; Deutsch, U.; Gruss, P.; Balling, R.; Hansmann, I.: PAX1, a member of the paired box-containing class of developmental control genes, is mapped to human chromosome 20p11.2 by in situ hybridization (ISH and FISH). Genomics 14: 740-744, 1992.
Ahmad, F.; Azevedo, J. L., Jr.; Cortright, R.; Dohm, G. L.; Goldstein, B. J.: Alternations in skeletal muscle protein-tyrosine phosphatase activity and expression in insulin-resistant human obesity and diabetes. J. Clin. Invest. 100: 449-458, 1997.
Brown-Shimer, S.; Johnson, K. A.; Lawrence, J. B.; Johnson, C.; Bruskin, A.; Green, N. R.; Hill, D. E.: Molecular cloning and chromosome mapping of the human gene encoding protein phosphotyrosyl phosphatase 1B. Proc. Nat. Acad. Sci. 87: 5148-5152, 1990.
Charbonneau, H.; Tonks, N. K.; Kumar, S.; Diltz, C. D.; Harrylock, M.; Cool, D. E.; Krebs, E. G.; Fischer, E. H.; Walsh, K. A.: Human placenta protein-tyrosine-phosphatase: amino acid sequence and relationship to a family of receptor-like proteins. Proc. Nat. Acad. Sci. 86:5252-5256, 1989.
Chernoff, J.; Schievella, A. R.; Jost, C. A.; Erikson, R. L.; Neel, B. G.: Cloning of a cDNA for a major human protein-tyrosine-phosphatase. Proc. Nat. Acad. Sci. 87: 2735-2739, 1990.
Mastick, C. C.; Brady, M. J.; Saltiel, A. R.: Insulin stimulates the tyrosine phosphorylation of caveolin. J. Cell Biol. 129: 1523-1531, 1995.
Soubeyran, P.; Kowanetz, K.; Szymkiewicz, I.; Langdon, W. Y.; Dikic, I.: Cbl-CIN85-endophilin complex mediates ligand-induced downregulation of EGF receptors. Nature 416: 183-187, 2002.
Nakamura, M.; Nagano, T.; Chikama, T.; Nishida, T.: Role of the small GTP-binding protein Rho in epithelial cell migration in the rabbit cornea. Invest. Ophthal. Vis. Sci. 42: 941-947, 2001.
Rao, P. V.; Deng, P.-F.; Kumar, J.; Epstein, D. L.: Modulation of aqueous humor outflow facility by the Rho kinase-specific inhibitor Y-27632. Invest. Ophthal. Vis. Sci. 42: 1029-1037, 2001.
Gharib, B.; Fox, M. F.; Bartoli, C.; Giorgi, D.; Sansonetti, A.; Swallow, D. M.; Dagorn, J. C.; Berge-Lefranc, J. L.: Human regeneration protein/lithostathine genes map to chromosome 2p12. Ann. Hum. Genet. 57:9-16, 1993.
Miyashita, H.; Nakagawara, K.; Mori, M.; Narushima, Y.; Noguchi, N.; Moriizumi, S.; Takasawa, S.; Yonekura, H.; Takeuchi, T.; Okamoto, H.: Human REG family genes are tandemly ordered in a 95-kilobase region of chromosome 2p12. FEBS Lett. 377: 429-433, 1995.
Moriizumi, S.; Watanabe, T.; Unno, M.; Nakagawara, K.; Suzuki, Y.; Miyashita, H.; Yonekura, H.; Okamoto, H.: Isolation, structural determination and expression of a novel reg gene, human regl-beta. Biochim. Biophys. Acta 1217: 199-202, 1994.
Holmes, C.; Arranz, M. J.; Powell, J. F.; Collier, D. A.; Lovestone, S.: 5-HT-2A and 5-HT-2C receptor polymorphisms and psychopathology in late onset Alzheimer's disease. Hum. Molec. Genet. 7: 1507-1509, 1998.
Grueneberg, D. A.; Natesan, S.; Alexandre, C.; Gilman, M. Z.: Human and Drosophila homeo domain proteins that enhance the DNA-binding activity of serum response factor. Science 257: 1089-1095, 1992.
Kern, M. J.; Argao, E. A.; Birkenmeier, E. H.; Rowe, L. B.; Potter, S. S.: Genomic organization and chromosome localization of the murine homeobox gene Pmx. Genomics 19: 334-340, 1994.
Nakamura, T.; Yamazaki, Y.; Hatano, Y.; Miura, I.: NUP98 is fused to PMX1 homeobox gene in human acute myelogenous leukemia with chromosome translocation t(1;11)(q23;p15). Blood 94: 741-747, 1999.
Norris, R. A.; Scott, K. K.; Moore, C. S.; Stetten, G.; Brown, C. R.; Jabs, E. W.; Wulfsberg, E. A.; Yu, J.; Kern, M. J.: Human PRRX1 and PRRX2 genes: cloning, expression, genomic localization, and exclusion as disease genes for Nager syndrome. Mammalian Genome 11:1000-1005, 2000.
Riazuddin, S.; Castelein, C. M.; Ahmed, Z. M.; Lalwani, A. K.; Mastroianni, M. A.; Naz, S.; Smith, T. N.; Liburd, N. A.; Friedman, T. B.; Griffith, A. J.; Riazuddin, S.; Wilcox, E. R.: Dominant modifier DFNM1 suppresses recessive deafness DFNB26. Nature Genet. 26: 431-434, 2000.
Coleman, M. P.; Ambrose, H. J.; Carrel, L.; Nemeth, A. H.; Willard, H. F.; Davies, K. E.: A novel gene, DXS8237E, lies within 20 kb upstream of UBEL in Xp11.23 and has a different X inactivation status. Genomics 31:135-138, 1996.
Inoue, A.; Takahashi, K. P.; Kimura, M.; Watanabe, T.; Morisawa, S.: Molecular cloning of a RNA binding protein, S1-1. Nucleic Acids Res. 24: 2990-2997, 1996.
Nagase, T.; Seki, N.; Tanaka, A.; Ishikawa, K.; Nomura, N.: Prediction of the coding sequences of unidentified human genes. IV. The coding sequences of 40 new genes (KIAA0121-KIAA0160) deduced by analysis of cDNA clones from human cell line KG-1. DNA Res. 2: 167-174, 1995.
Lin, C.-S.; Aebersold, R. H.; Leavitt, J.: Correction of the N-terminal sequences of the human plastin isoforms by using anchored polymerase chain reaction: identification of a potential calcium-binding domain. Molec. Cell. Biol. 10: 1818-1821, 1990
Lahn, B. T.; Page, D. C.: Functional coherence of the human Y chromosome. Science 278: 675-680, 1997.
Barletta, C.; Druck, T.; LaForgia, S.; Calabretta, B.; Drabkin, H.; Patterson, D.; Croce, C. M.; Huebner, K.: Chromosome locations of the MYB related genes, AMYB and BMYB. Cancer Res. 51: 3821-3824, 1991.
Nomura, N.; Takahashi, M.; Matsui, M.; Ishii, S.; Date, T.; Sasamoto, S.; Ishizaki, R.: Isolation of human cDNA clones of MYB-related genes, A-MYB and B-MYB. Nucleic Acids Res. 16: 11075-11089, 1988.
Takahashi, T.; Nakagoshi, H.; Sarai, A.; Nomura, N.; Yamamoto, T.; Ishii, S.: Human A-myb gene encodes a transcriptional activator containing the negative regulatory domains. FEBS Lett. 358: 89-96, 1995.
Ellis, N. A.: Ecce Ohno. Nature Genet. 10: 373-375, 1995.
Haldane, J. B. S.: Sex ratio and unisexual sterility in hybrid animals. J. Genet. 12: 101-109, 1922.
Milatovich, A.; Kitamura, T.; Miyajima, A.; Francke, U.: Gene for the alpha-subunit of the human interleukin-3 receptor (IL3RA) localized to the X-Y pseudoautosomal region. Am. J. Hum. Genet. 53:1146-1153, 1993.
Ohno, S.: Sex Chromosomes and Sex-linked Genes. Berlin and New York: Springer (pub.) 1967
Palmer, S.; Perry, J.; Ashworth, A.: A contravention of Ohno's law in mice. Nature Genet. 10: 472-476, 1995.
Rugarli, E. I.; Adler, D. A.; Borsani, G.; Tsuchiya, K.; Franco, B.; Hauge, X.; Disteche, C.; Chapman, V.; Ballabio, A.: Different chromosomal localization of the Clcn4 gene in Mus spretus and C57BL/6J mice. Nature Genet. 10: 466-471, 1995.
Schnur, R. E.; Wick, P. A.: Intragenic Taql restriction fragment length polymorphism (RFLP) in CLCN4, between the loci for X-linked ocular albinism (OA1) and microphthalmia with linear skin defects syndrome (MLS). Hum. Genet. 95: 594-595, 1995.
van Slegtenhorst, M. A.; Bassi, M. T.; Borsani, G.; Wapenaar, M. C.; Ferrero, G. B.; de Conciliis, L.; Rugarli, E. I.; Grillo, A.; Franco, B.; Zoghbi, H. Y.; Ballabio, A.: A gene from the Xp22.3 region shares homology with voltage-gated chloride channels. Hum. Molec. Genet. 3: 547-552, 1994.
Grandori, C.; Mac, J.; Siebelt, F.; Ayer, D. E.; Eisenman, R. N.: Myc-Max heterodimers activate a DEAD box gene and interact with multiple E box-related sites in vivo. EMBO J. 15: 4344-4357, 1996.
Prendergast, G. C.; Lawe, D.; Ziff, E. B.: Association of Myn, the murine homolog of Max, with c-Myc stimulates methylation-sensitive DNA binding and Ras cotransformation. Cell 65: 395-408, 1991.
Wagner, A. J.; Le Beau, M. M.; Diaz, M. O.; Hay, N.: Expression, regulation, and chromosomal localization of the Max gene. Proc. Nat. Acad. Sci. 89: 3111-3115, 1992.
Zervos, A. S.; Faccio, L.; Gatto, J. P.; Kyriakis, J. M.; Brent, R.: Mxi2, a mitogen-activated protein kinase that recognizes and phosphorylates Max protein. Proc. Nat. Acad. Sci. 92: 10531-10534, 1995.
Bonner, T. I.; Young, A. C.; de Miguel, C.; Detera-Wadleigh, S.; Modi, W. S.; O'Brien, S. J.: The chromosomal location of the two human tackykin in (sic) genes: human substance P and neurokinin K. (Abstract) Cytogenet. Cell Genet. 46: 584 only, 1987.
Cao, Y. Q.; Mantyh, P. W.; Carlson, E. J.; Gillespie, A.-M.; Epstein, C. J.; Basbaum, A. I.: Primary afferent tachykinins are required to experience moderate to intense pain. Nature 392: 390-393, 1998.
Krause, J. E.; Chirgwin, J. M.; Carter, M. S.; Xu, Z. S.; Hershey, A. D.: Three rat preprotachykinin mRNAs encode the neuropeptides substance P and neurokinin A. Proc. Nat. Acad. Sci. 84: 881-885, 1987
Liu, H.; Cao, Y.; Basbaum, A. I.; Mazarati, A. M; Sankar, R.; Wasterlain, C. G.: Resistance to excitotoxin-induced seizures and neuronal death in mice lacking the preprotachykinin A gene. Proc. Nat. Acad. Sci. 96:12096-12101, 1999.
Troger, J.; Neyer, S.; Heufler, C.; Huemer, H.; Schmid, E.; Griesser, U.; Kralinger, M.; Kremser, B.; Baldissera, I.; Kieselbach, G.: Substance P and vasoactive intestinal polypeptide in the streptozotocin-induced diabetic rat retina. Invest. Ophthal. Vis. Sci. 42: 1045-1050, 2001.
Zimmer, A.; Zimmer, A. M.; Baffi, J.; Usdin, T.; Reynolds, K.; Konig, M.; Palkovits, M.; Mezey, E.: Hypoalgesia in mice with a targeted deletion of the tachykinin 1 gene. Proc. Nat. Acad. Sci. 95: 2630-2635, 1998.
Mattei, M.-G.; Riviere, M.; Krust, A.; Ingvarsson, S.; Vennstrom, B.; Islam, M. Q.; Levan, G.; Kautner, P.; Zelent, A.; Chambon, P.; Szpirer, J.; Szpirer, C.: Chromosomal assignment of retinoic acid receptor (RAR) genes in the human, mouse, and rat genomes. Genomics 10:1061-1069, 1991.
Abramson, D. H.; Ellsworth, R. M.; Zimmerman, L. E.: Monocular cancer in retinoblastoma survivors. Trans. Am. Acad. Ophthal. Otolaryng. 81:454-457, 1976
Aherne, G. E. S.; Roberts, D. F.: Retinoblastoma—a clinical survey and its genetic implications. Clin. Genet. 8: 275-290, 1975.
Alonso, J.; Garcia-Miguel, P.; Abelairas, J.; Mendiola, M.; Sarret, E.; Vendrell, M. T.; Navajas, A.; Pestana, A.: Spectrum of germline RB1 gene mutations in Spanish retinoblastoma patients: phenotypic and molecular epidemiological implications. Hum. Mutat. 17: 412-422, 2001.
Amoaku, W. M. K.; Willshaw, H. E.; Parkes, S. E.; Shah, K. J.; Mann, J. R.: Trilateral retinoblastoma: a report of five patients. Cancer 78:858-863, 1996.
Bader, J. L.; Meadows, A. T.; Zimmerman, L. E.; Rorke, L. B.; Voute, P. A.; Champion, L. A. A.; Miller, R. W.: Bilateral retinoblastoma with ectopic intracranial retinoblastoma: trilateral retinoblastoma. Cancer Genet. Cytogenet. 5: 203-213, 1982.
Balaban-Malenbaum, G.; Gilbert, F.; Nichols, W. W.; Hill, R.; Shields, J.; Meadows, A. T.: A deleted chromosome no. 13 in human retinoblastoma cells: relevance to tumorigenesis. Cancer Genet. Cytogenet. 3: 243-250, 1981.
Bandara, L. R.; Adamczewski, J. P.; Hunt, T.; La Thangue, N. B.: Cyclin A and the retinoblastoma gene product complex with a common transcription factor. Nature 352: 249-251, 1991.
Benedict, W. F.; Murphree, A. L.; Banerjee, A.; Spina, C. A.; Sparkes, M. C.; Sparkes, R. S.: Patient with 13 chromosome deletion: evidence that the retinoblastoma gene is a recessive cancer gene. Science 219:973-975, 1983.
Benedict, W. F.; Xu, H.-J.; Hu, S.-X.; Takahashi, R.: Role of the retinoblastoma gene in the initiation and progression of human cancer. J. Clin. Invest. 85: 988-993, 1990.
Bia, B.; Cowell, J. K.: Independent constitutional germline mutations occurring in the RB1 gene in cousins with bilateral retinoblastoma. Oncogene 11:977-979, 1995.
Blanquet, V.; Turleau, C.; de Grouchy, J.; Creau-Goldberg, N.: Physical map around the retinoblastoma gene: possible genomic imprinting suggested by NruI digestion. Genomics 10: 350-355, 1991.
Blanquet, V.; Turleau, C.; Gross-Morand, M. S.; Senamaud-Beaufort, C.; Doz, F.; Besmond, C.: Spectrum of germline mutations in the RB1 gene: a study of 232 patients with hereditary and non hereditary retinoblastoma. Hum. Molec. Genet. 4: 383-388, 1995.
Nicklin, M. J. H.; Weith, A.; Duff, G. W.: A physical map of the region encompassing the human interleukin-1-alpha, interleukin-1-beta, and interleukin-1 receptor antagonist genes. Genomics 19: 382-384, 1994.
Ansano, M.; Toda, M.; Sakaguchi, N.; Sakaguchi, S.: Autoimmune disease as a consequence of developmental abnormality of a T cell subpopulation. J. Exp. Med. 184: 387-396, 1996.
Cosman, D.; Cerretti, D. P.; Larsen, A.; Park, L.; March, C.; Dower, S.; Gillis, S.; Urdal, D.: Cloning, sequence and expression of human interleukin-2 receptor. Nature 312: 768-771, 1984.
Ferrari, S.; Cannizzaro, L. A.; Battini, R.; Huebner, K.; Baserga, R.: The gene encoding human vimentin is located on the short arm of chromosome 10. Am. J. Hum. Genet. 41: 616-626, 1987.
Greene, W. C.; Leonard, W. J.; Depper, J. M.; Nelson, D. L.; Waldmann, T. A.: The human interleukin-2 receptor: normal and abnormal expression in T cells and in leukemias induced by the human T-lymphotropic retroviruses. Ann. Intern. Med. 105: 560-572, 1986.
Hatakeyama, M.; Minamoto, S.; Taniguchi, T.: Intracytoplasmic phosphorylation sites of Tac antigen (p55) are not essential for the conformation, function, and regulation of the human interleukin 2 receptor. Proc. Nat. Acad. Sci. 83: 9650-9654, 1986.
Hatakeyama, M.; Minamoto, S.; Uchiyama, T.; Hardy, R. R.; Yamada, G.; Taniguchi, T.: Reconstitution of functional receptor for human interleukin-2 in mouse cells. Nature 318: 467-470, 1985.
Ihle, J. N.; Kerr, I. M.: Jaks and Stats in signaling by the cytokine receptor super family. Trends Genet. 11: 69-74, 1995.
Ishida, N.; Kanamori, H.; Noma, T.; Nikaido, T.; Sabe, H.; Suzuki, N.; Shimizu, A.; Honjo, T.: Molecular cloning and structure of the human interleukin 2 receptor gene. Nucleic Acids Res. 13: 7579-7589, 1985.
Kondo, S.; Shimizu, A.; Maeda, M.; Tagaya, Y.; Yodoi, J.; Honjo, T.: Expression of functional human interleukin-2 receptor in mouseT cells by cDNA transfection. Nature 320: 75-77, 1986.
Leonard, W. J.; Depper, J. M.; Crabtree, G. R.; Rudikoff, S.; Pumphrey, J.; Robb, R. J.; Kronke, M.; Svetlik, P. B.; Peffer, N. J.; Waldmann, T. A.; Greene, W. C.: Molecular cloning and expression of cDNAs for the human interleukin-2 receptor. Nature 311: 626-631, 1984.
Chen, T.-R.; McMorris, F. A.; Creagan, R.; Ricciuti, F. C.; Tischfield, J.; Ruddle, F. H.: Assignment of the genes for malate oxidoreductasede carboxylating to chromosome 6 and peptidase B and lactate dehydrogenase B to chromosome 12 in man. Am. J. Hum. Genet. 25: 200-207, 1973.
Hamerton, J. L.; Mohandas, T.; McAlpine, P. J.; Douglas, G. R.: Localization of human gene loci using spontaneous chromosome rearrangements in human-Chinese hamster somatic cell hybrids. Am. J. Hum. Genet. 27:595-608, 1975.
Herbschleb-Voogt, E.; Meera Khan, P.: Defining the locus of origin of a genetically determined electrophoretic variant of a multilocus enzyme system; the Calcutta-1 of human LDH system is a B-locus variant. Hum. Genet. 57: 290-295, 1981.
Houki, N.; Matsushima, Y.; Kitamura, M.; Tukada, T.; Nishina, T.; Nakayama, T.: A case of deficiency of lactate dehydrogenase H-subunit. Jpn. J. Clin. Chem. 15: 85-90, 1986.
Kitamura, M.; Iijima, N.; Hashimoto, F.; Hiratsuka, A.: Hereditary deficiency of subunit H of lactate dehydrogenase. Clin. Chim. Acta 34:419-423, 1971.
Maekawa, M.; Sudo, K.; Kitajima, M.; Matsuura, Y.; Li, S. S.-L.; Kanno, T.: Analysis of a genetic mutation in an electrophoretic variant of the human lactate dehydrogenase B(H) subunit. Hum. Genet. 91:423-426, 1993.
Maekawa, M.; Sudo, K.; Nagura, K.; Li, S. S.-L.; Kanno, T.: Population screening of lactate dehydrogenase deficiencies in Fukuoka Prefecture in Japan and molecular characterization of three independent mutations in the lactate dehydrogenase-B(H) gene. Hum. Genet. 93: 74-76, 1994.
Malpuech, G.; Kaplan, J. C.; Rethore, M. O.; Junien, C.; Geneix, A.: Une observation de deletion partielle du bras court du chromosome 12: localisation du gene de la lacticodes hydrogenase B. Lyon Med. 233:275-279, 1975.
Mayeda, K.; Weiss, L.; Lindahl, R.; Dully, M.: Localization of the human lactate dehydrogenase B gene on the short arm of chromosome 12. Am. J. Hum. Genet. 26: 59-64, 1974.
Miwa, S.; Nishima, T.; Kanehashi, Y.; Kitamura, M.; Hiratsuka, A.; Shizume, K.: Studies on erythrocyte metabolism in a case with hereditary deficiency of H-subunit of lactate dehydrogenase. Acta Haemat. Jpn. 34: 228-232, 1971.
Rasmussen, S. K.; Lautier, C.; Hansen, L.; Echwald, S. M.; Hansen, T.; Ekstrom, C. T.; Urhammer, S. A.; Borch-Johnsen, K.; Grigorescu, F.; Smith, R. J.; Pedersen, O.: Studies of the variability of the genes encoding the insulin-like growth factor I receptor and its ligand in relation to type 2 diabetes mellitus. J. Clin. Endocr. Metab. 85:1606-1610, 2000.
Leonard, W. J.; Depper, J. M.; Kanehisa, M.; Kronke, M.; Peffer, N. J.; Svetlik, P. B.; Sullivan, M.; Greene, W. C.: Structure of the human interleukin-2 receptor gene. Science 230: 633-639, 1985.
Leonard, W. J.; Depper, J. M.; Robb, R. J.; Waldmann, T. A.; Greene, W. C.: Characterization of the human receptor for T-cell growth factor. Proc. Nat. Acad. Sci. 80: 6957-6961, 1983.
Marx, J. L.: The interleukin-2 receptor gene is cloned. Science 226:1064-1065, 1985.
Nikaido, T.; Shimizu, A.; Ishida, N.; Sabe, H.; Teshigawara, K.; Maeda, M.; Uchiyama, T.; Yodoi, J.; Honjo, T.: Molecular cloning of a cDNA encoding human interleukin-2 receptor. Nature 311: 631-635, 1984.
Robb, R. J.; Rusk, C. M.; Neeper, M. P.: Structure-function relationships for the interleukin 2 receptor: location of ligand and antibody binding sites on the Tac receptor chain by mutational analysis. Proc. Nat. Acad. Sci. 85: 5654-5658, 1988.
Sharfe, N.; Dadi, H. K.; Shahar, M.; Roifman, C. M.: Human immune disorder arising from mutation of the alpha chain of the interleukin-2 receptor. Proc. Nat. Acad. Sci. 94: 3168-3171, 1997.
Shevach, E. M.: Certified professionals: CD4(+)CD25(+) suppressor T cells. J. Exp. Med. 193: F41-F45, 2001.
Tsudo, M.; Kozak, R. W.; Goldman, C. K.; Waldmann, T. A.: Demonstration of a non-Tac peptide that binds interleukin 2: a potential participant in a multichain interleukin 2 receptor complex. Proc. Nat. Acad. Sci. 83: 9694-9698, 1986.
Urdal, D. L.; March, C. J.; Gillis, S.; Larsen, A.; Dower, S. K.: Purification and chemical characterization of the receptor for interleukin 2 from activated human T lymphocytes and from a humanT-cell lymphoma cell line. Proc. Nat. Acad. Sci. 81: 6481-6485, 1984.
Mohrenweiser, H. W.; Neel, J. V.: Frequency of thermostability variants: estimation of total ‘rare’ variant frequency in human populations. Proc. Nat. Acad. Sci. 78: 5729-5733, 1981.
Okumura, N.; Terasawa, F.; Ueno, I.; Oki, K.; Yamauchi, K.; Hidaka, H.; Tozuka, M.; Okura, M.; Katsuyama, T.: Genetic analyses in homozygous and heterozygous variants of lactate dehydrogenase-B (H) subunit: LD-B Matsumoto I and II (LD-B W323R). Clin. Chim. Acta 287: 163-171, 1999
Rethore, M.-O.; Junien, C.; Malpuech, G.; Baccichetti, C.; Tenconi, R.; Kaplan, J. C.; de Romeuf, J.; Lejeune, J.: Localisation du genede la glyceraldehyde 3-phosphate dehydrogenase (G3PD) sur le segment distal du bras court du chromosome 12. Ann. Genet. 19: 140-142, 1976.
Rethore, M.-O.; Kaplan, J.-C.; Junien, C.; Cruveiller, J.; Dutrillaux, B.; Aurias, A.; Carpentier, S.; Lafourcade, J.; Lejeune, J.: Augmentationde l'activite de la LDH-B chez un garcon trisomique 12p par malsegregationd'une translocation maternelle t(12;14)(q12;p11). Ann. Genet. 18:81-87, 1975.
Sakai, I.; Sharief, F. S.; Pan, Y.-C. E.; Li, S. S.-L.: The cDNAand protein sequences of human lactate dehydrogenase B. Biochem. J. 248: 933-936, 1987.
Santachiara, A. S.; Nabholz, M.; Miggiano, V.; Darlington, A. J.; Bodmer, W. F.: Linkage between human lactate dehydrogenase Band peptidase B genes. Nature 227: 248-251, 1970.
Steinbach, P.; Rehder, H.: Tetrasomy for the short arm of chromosome 12 with accessory isochromosome (+i[12p]) and a marked LDH-B gene dosage effect. Clin. Genet. 32: 1-4, 1987.
Sudo, K.: Personal Communication. Komae City, Japan Jul. 12, 1993.
Sudo, K.; Maekawa, M.; Ikawa, S.; Machida, K.; Kitamura, M.; Li, S. S.-L.: A missense mutation found in human lactate dehydrogenase-B(H) variant gene. Biochem. Biophys. Res. Commun. 168: 672-676, 1990.
Sudo, K.; Maekawa, M.; Luedemann, M. M.; Deaven, L. L.; Li, S. S.-L.: Human lactate dehydrogenase-B processed pseudo gene: nucleotide sequence analysis and assignment to the X-chromosome. Biochem. Biophys. Res. Commun. 171: 67-74, 1990.
Sudo, K.; Maekawa, M.; Tomonaga, A.; Tsukada, T.; Nakayama, T.; Kitamura, M.; Li, S. S.-L.; Kanno, T.; Toriumi, J.: Molecular characterization of genetic mutations in human lactate dehydrogenase (LDH)B (H) variant. Hum. Genet. 89: 158-162, 1992.
Van Someren, H.; Meera Khan, P.; Westerveld, A.; Bootsma, D.: Human genetics—two new linkage groups carrying different loci for LDH and glutamic-pyruvic transaminase found. Nature 240: 221-222, 1972.
Weiss, L.; Mayeda, K.; Lindahl, R.; Dully, M.: Localization of human LDH-B gene of the short arm of chromosome 12. (Abstract) Am. J. Hum. Genet. 25: 85A only, 1973.
Rajagopalan, H.; Bardelli, A.; Lengauer, C.; Kinzler, K. W.; Vogelstein, B.; Velculescu, V. E.: RAF/RAS oncogenes and mismatch-repair status. (Letter) Nature 418: 934 only, 2002.
Zhong, S.; Delva, L.; Rachez, C.; Cenciarelli, C.; Gandini, D.; Zhang, H.; Kalantry, S.; Freedman, L. P.; Pandolfi, P. P.: A RA-dependent, tumour-growth suppressive transcription complex is the target of the PML-RAR-alpha and T18 oncoproteins. Nature Genet. 23: 287-295, 1999.
Fukuyama, R.; Ichijoh, Y.; Minoshima, S.; Kitamura, N.; Shimizu, N.: Assignment of hepatocyte growth factor (HGF) gene to chromosome 7q21.1. (Abstract) Cytogenet. Cell Genet. 58: 1921 only, 1991.
Fukuyama, R.; Ichijoh, Y.; Minoshima, S.; Kitamura, N.; Shimizu, N.: Regional localization of the hepatocyte growth factor (HGF) geneto human chromosome 7 band q21.1. Genomics 11: 410-415, 1991.
Gherardi, E.; Stoker, M.: Hepatocytes and scatter factor. Nature 346:228 only, 1990.
Gohda, E.; Tsubouchi, H.; Nakayama, H.; Hirono, S.; Sakiyama, O.; Takahashi, K.; Miyazaki, H.; Hashimoto, S.; Daikuhara, Y.: Purification and partial characterization of hepatocyte growth factor from plasma of a patient with fulminant hepatic failure. J. Clin. Invest. 81:414-419, 1988.
Kilby, M. D.; Afford, S.; Li, X. F.; Strai n, A. J.; Ah med, A.; Whittle, M. J.: Localisation of hepatocyte growth factor and its receptor (c-met) protein and mRNA in human term placenta. Growth Factors 13: 133-139, 1996.
Lai, L.; Goldschneider, I.: Cutting edge: identification of a hybrid cytokine consisting of IL-7 and the beta-chain of the hepatocyte growth factor/scatter factor. J. Immun. 167: 3550-3554, 2001.
Maina, F.; Casagranda, F.; Audero, E.; Simeone, A.; Comoglio, P. M.; Klein, R.; Ponzetto, C.: Uncoupling of Grb2 from the Met receptor in vivo reveals complex roles in muscle development. Cell 87: 531-542, 1996.
Miyazawa, K.; Tsubouchi, H.; Naka, D.; Takahashi, K.; Okigaki, M.; Arakaki, N.; Nakayama, H.; Hirono, S.; Sakiyama, O.; Takahashi, K.; Gohda, E.; Daikuhara, Y.; Kitamura, N.: Molecular cloning and sequence analysis of cDNA for human hepatocyte growth factor. Biochem. Biophys. Res. Commun. 163: 967-973, 1989.
Nakamura, T.; Nishizawa, T.; Hagiya, M.; Seki, T.; Shimonishi, M.; Sugimura, A.; Tashiro, K.; Shimizu, S.: Molecular cloning and expression of human hepatocyte growth factor. Nature 342: 440-443, 1989. Noonan, F. P.; Recio, J. A.; Takayama, H.; Duray, P.; Anver, M. R.; Rush, W. L.; De Fabo, E. C.; Merlino, G.: Neonatal sunburn and melanoma in mice: severe sunburn in newborn, but not adult, mice is linked with melanoma in later life. Nature 413: 271-272, 2001.
Powell, E. M.; Mars, W. M.; Levitt, P.: Hepatocyte growth factor/scatter factor is a motogen for interneurons migrating from the ventral to dorsal telencephalon. Neuron 30: 79-89, 2001.
Rubin, J. S.; Chan, A. M.-L.; Bottaro, D. P.; Burgess, W. H.; Taylor, W. G.; Cech, A. C.; Hirschfield, D. W.; Wong, J.; Miki, T.; Finch, P. W.; Aaronson, S. A.: A broad-spectrum human lung fibroblast-derived mitogen is a variant of hepatocyte growth factor. Proc. Nat. Acad. Sci. 88: 415-419, 1991.
Saccone, S.; Narsimhan, R. P.; Gaudino, G.; Dalpra, L.; Comoglio, P. M.; Della Valle, G.: Regional mapping of the human hepatocyte growth factor (HGF)-scatter factor gene to chromosome 7q21.1. Genomics 13:912-914, 1992.
Schmidt, C.; Bladt, F.; Goedecke, S.; Brinkmann, V.; Zschiesche, W.; Sharpe, M.; Gherardi, E.; Birchmeier, C.: Scatter factor/hepatocyte growth factor is essential for liver development. Nature 373: 699-702, 1995
Uehara, Y.; Minowa, O.; Mori, C.; Shiota, K.; Kuno, J.; Noda, T.; Kitamura, N.: Placental defect and embryonic lethality in mice lacking hepatocyte growth factor/scatter factor. Nature 373: 702-705, 1995.
Weidner, K. M.; Arakaki, N.; Hartmann, G.; Vandekerckhove, J.; Weingart, S.; Rieder, H.; Fonatsch, C.; Tsubouchi, H.; Hishida, T.; Daikuhara, Y.; Birchmeier, W.: Evidence for the identity of human scatter factor and human hepatocyte growth factor. Proc. Nat. Acad. Sci. 88: 7001-7005, 1991.
Zarnegar, R.; Petersen, B.; DeFrances, M. C.; Michalopoulos, G.: Localization of hepatocyte growth factor (HGF) gene on human chromosome 7. Genomics 12: 147-150, 1992.
Lafuse, W. P.; Zwilling, B. S.: Localization of the inhibin beta-Bgene on mouse chromosome 1. Mammalian Genome 4: 399-400, 1993.
Salmenkivi, K.; Arola, J.; Voutilainen, R.; Ilvesmaki, V.; Haglund, C.; Kahri, A. I.; Heikkila, P.; Liu, J.: Inhibin/activin beta-B-subunit expression in pheochromocytomas favors benign diagnosis. J. Clin. Endocr. Metab. 86: 2231-2235, 2001.
Schrewe, H.; Gendron-Maguire, M.; Harbison, M. L.; Gridley, T.: Mice homozygous for a null mutation of activin beta B are viable and fertile. Mech. Dev. 47: 43-51, 1994.
Nicoll, J. A. R.; Mrak, R. E.; Graham, D. I.; Stewart, J.; Wilcock, G.; MacGowan, S.; Esiri, M. M.; Murray, L. S.; Dewar, D.; Love, S.; Moss, T.; Griffin, W. S. T.: Association of interleukin-1 gene polymorphisms with Alzheimer's disease. Ann. Neurol. 47: 365-368, 2000.
Rogers, J.: An IL-1-alpha susceptibility polymorphism in Alzheimer's disease: new fuel for the inflammation hypothesis. (Editorial) Neurology 55:464-465, 2000.
Sabatino, M.; Boyce, B.; Aufdemorte, T.; Bonewald, L.; Mundy, G. R.: Infusions of recombinant human interleukins 1 alpha and 1 beta cause hypercalcemia in normal mice. Proc. Nat. Acad. Sci. 85:5235-5239, 1988.
Silver, A. R. J.; Masson, W. K.; George, A. M.; Adam, J.; Cox, R.: The Il-1 alpha and beta genes are closely linked (less than 70 kb) on mouse chromosome 2. Somat. Cell Molec. Genet. 16: 549-556, 1990.
Tanaka, A. R.; Ikeda, Y.; Abe-Dohmae, S.; Arakawa, R.; Sadanami, K.; Kidera, A.; Nakagawa, S.; Nagase, T.; Aoki, R.; Kioka, N.; Amachi, T.; Yokoyama, S.; Ueda, K.: Human ABCA1 contains a large amino terminal extracellular domain homologous to an epitope of Sjogren's syndrome. Biochem. Biophys. Res. Commun. 283: 1019-1025, 2001
Chesi, M.; Nardini, E.; Brents, L. A.; Schrock, E.; Ried, T.; Kuehl, W. M.; Bergsagel, P. L.: Frequent translocation t(4;14)(p16.3;q32.3) in multiple myeloma is associated with increased expression and activating mutations of fibroblast growth factor receptor 3. Nature Genet. 16:260-264, 1997.
Baroni, M. G.; Oelbaum, R. S.; Pozzilli, P.; Stocks, J.; Li, S.-R.; Fiore, V.; Galton, D. J.: Polymorphisms at the GLUT1 (HepG2) and GLUT4 (muscle/adipocyte) glucose transporter genes and non-insulin-dependent diabetes mellitus (NIDDM). Hum. Genet. 88: 557-561, 1992.
Kelley, M. J.; Pech, M.; Seuanez, H. N.; Rubin, J. S.; O'Brien, S. J.; Aaronson, S. A.: Emergence of the keratinocyte growth factor multi gene family during the great ape radiation. Proc. Nat. Acad. Sci. 89: 9287-9291, 1992.
Mattei, M.-G.; de Lapeyriere, O.; Bresnick, J.; Dickson, C.; Birnbaum, D.; Mason, I.: Mouse Fgf7 (fibroblast growth factor 7) and Fgf8 (fibroblast growth factor 8) genes map to chromosomes 2 and 19 respectively. Mammalian Genome 6: 196-197, 1995.
Rubin, J. S.; Osada, H.; Finch, P. W.; Taylor, W. G.; Rudikoff, S.; Aaronson, S. A.: Purification and characterization of a newly identified growth factor specific for epithelial cells. Proc. Nat. Acad. Sci. 86: 802-806, 1989.
Werner, S.; Smola, H.; Liao, X.; Longaker, M. T.; Krieg, T.; Hofschneider, P. H.; Williams, L. T.: The function of KGF in morphogenesis of epithelium and reepithelialization of wounds. Science 266: 819-822, 1994.
Zimonjic, D. B.; Kelley, M. J.; Rubin, J. S.; Aaronson, S. A.; Popescu, N. C.: Fluorescence in situ hybridization analysis of keratinocyte growth factor gene amplification and dispersion in evolution of greatapes and humans. Proc. Nat. Acad. Sci. 94: 11461-11465, 1997.
Nagase, T.; Seki, N.; Ishikawa, K.; Tanaka, A.; Nomura, N.: Prediction of the coding sequences of unidentified human genes. V. The coding sequences of 40 new genes (KIAA0161-KIAA0200) deduced by analysis of cDNA clones from human cell line KG-1. DNA Res. 3: 17-24, 1996.
Bassi, M. T.; Ramesar, R. S.; Caciotti, B.; Winship, I. M.; DeGrandi, A.; Riboni, M.; Townes, P. L.; Beighton, P.; Ballabio, A.; Borsani, G.: X-linked late-onset sensorineural deafness caused by a deletion involving OA1 and a novel gene containing WD-40 repeats. Am. J. Hum. Genet. 64: 1604-1616, 1999.
Converse, P. J.: Personal Communication. Baltimore, Md. Aug. 24, 2001
Disteche, C. M.; Dinulos, M. B.; Bassi, M. T.; Elliott, R. W.; Rugarli, E. I.: Mapping of the murine Tbl1 gene reveals a new rearrangement between mouse and human X chromosomes. Mammalian Genome 9: 1062-1064, 1998.
Dong, X.; Tsuda, L.; Zavitz, K. H.; Lin, M.; Li, S.; Carthew, R. W.; Zipursky, S. L.: ebi regulates epidermal growth factor receptor signaling pathways in Drosophila. Genes Dev. 13: 954-965, 1999.
Matsuzawa, S.; Reed, J. C.: Siah-1, SIP, and Ebi collaborate in a novel pathway for beta-catenin degradation linked to p53 responses. Molec. Cell 7: 915-926, 2001.
Zhang, J.; Kalkum, M.; Chait, B. T.; Roeder, R. G.: The N-CoR-HDAC3 nuclear receptor corepressor complex inhibits the JNK pathway through the integral subunit GPS2. Molec. Cell 9: 611-623, 2002.
Farndon, J. R.; Leight, G. S.; Dilley, W. G.; Baylin, S. B.; Small ridge, R. C.; Harrison, T. S.; Wells, S. A., Jr.: Familial medullary thyroid carcinoma without associated endocrinopathies: a distinct clinical entity. Brit. J. Surg. 73: 278-281, 1986.
Ellis, S. A.; Palmer, M. S.; McMichael, A. J.: Human trophoblast and the choriocarcinoma cell line BeWo express a truncated HLA class I molecule. J. Immun. 144: 731-735, 1990.
Geraghty, D. E.; Koller, B. H.; Orr, H. T.: A human major histocompatibility complex class I gene that encodes a protein with a shortened cytoplasmic segment. Proc. Nat. Acad. Sci. 84: 9145-9149, 1987.
Geraghty, D. E.; Pei, J.; Lipsky, B.; Hansen, J. A.; Taillon-Miller, P.; Bronson, S. K.; Chaplin, D. D.: Cloning and physical mapping of the HLA class I region spanning the HLA-E-to-HLA-F interval by using yeast artificial chromosomes. Proc. Nat. Acad. Sci. 89: 2669-2673, 1992.
Hurks, H. M. H.; Valter, M. M.; Wilson, L.; Hilgert, I.; van denElsen, P. J.; Jager, M. J.: Uveal melanoma: no expression of HLA-G. Invest. Ophthal. Vis. Sci. 42: 3081-3084, 2001.
Kirszenbaum, M.; Moreau, P.; Gluckman, E.; Dausset, J.; Carosella, E.: An alternatively spliced form of HLA-G mRNA in human trophoblasts and evidence for the presence of HLA-G transcript in adult lymphocytes. Proc. Nat. Acad. Sci. 91: 4209-4213, 1994.
Lila, N.; Carpentier, A.; Amrein, C.; Khalil-Daher, I.; Dausset, J.; Carosella, E. D.: Implication of HLA-G molecule in heart-graft acceptance. Lancet 355: 2138 only, 2000.
McAlpine, P. J.: Personal Communication. Winnipeg, Manitoba, Canada Jun. 22, 1988.
Morales, P.; Corell, A.; Martinez-Laso, J.; Martin-Villa, J. M.; Varela, P.; Paz-Artal, E.; Allende, L.-M.; Arnaiz-Villena, A.: Three new HLA-G alleles and their linkage disequilibria with HLA-A. Immunogenetics 38:323-331, 1993.
Onno, M.; Guillaudeux, T.; Amiot, L.; Renard, I.; Drenou, B.; Hirel, B.; Girr, M.; Semana, G.; Le Bouteiller, P.; Fauchet, R.: The HLA-G gene is expressed at a low mRNA level in different human cells and tissues. Human Immun. 41: 79-86, 1994.
Paul, P.; Rouas-Freiss, N.; Khalil-Daher, I.; Moreau, P.; Riteau, B.; Le Gal, F. A.; Avril, M. F.; Dausset, J.; Guillet, J. G.; Carosella, E. D.: HLA-G expression in melanoma: a way for tumor cells to escape from immunosurveillance. Proc. Nat. Acad. Sci. 95: 4510-4515, 1998.
Rouas-Freiss, N.; Marchal, R. E.; Kirszenbaum, M.; Dausset, J.; Carosella, E. D.: The alpha1 domain of HLA-G1 and HLA-G2 inhibits cytotoxicity induced by natural killer cells: is HLA-G the public ligand for natural killer cell inhibitory receptors? Proc. Nat. Acad. Sci. 94: 5249-5254, 1997.
Schmidt, C. M.; Orr, H. T.: A physical linkage map of HLA-A, -G, -7.5p, and —F. Hum. Immun. 31: 180-185, 1991.
Heisler, L. K.; Cowley, M. A.; Tecott, L. H.; Fan, W.; Low, M. J.; Smart, J. L.; Rubinstein, M.; Tatro, J. B.; Marcus, J. N.; Holstege, H.; Lee, C. E.; Cone, R. D.; Elmquist, J. K.: Activation of central melanocortin pathways by fenfluramine. Science 297: 609-611, 2002.
Swan, D. C.; McBride, O. W.; Robbins, K. C.; Keithley, D. A.; Reddy, E. P.; Aaronson, S. A.: Chromosomal mapping of the simian sarcoma virus one gene analogue in human cells. Proc. Nat. Acad. Sci. 79: 4691-4695, 1982.
Guru, S. C.; Agarwal, S. K.; Manickam, P.; Olufemi, S.-E.; Crabtree, J. S.; Weisemann, J. M.; Kester, M. B.; Kim, Y. S.; Wang, Y.; Emmert-Buck, M. R.; Liotta, L. A.; Spiegel, A. M.; Boguski, M. S.; Roe, B. A.; Collins, F. S.; Marx, S. J.; Burns, L.; Chandrasekharappa, S. C.: A transcript map for the 2.8-Mb region containing the multiple endocrine neoplasia type 1 locus. Genome Res. 7: 725-735, 1997.
Van Leeuwen, A.: Di-allelic allo-antigenic systems on human T-lymphocyte subsets. Thesis: London Hospital Medical College (pub.) 1982.
Walker, I. D.; Sandrin, M. S.; Hogarth, P. M.; Sutton, V. R.; McKenzie, I. F. C.: Expression of Qa alloantigens on peripheral T-cells: the relationship of the Qa-m2, 7, 8, 9 specificities. Immunogenetics 24:90-94, 1986.
Yokoyama, W. M.: The mother-child union: the case of missing-self and protection of the fetus. Proc. Nat. Acad. Sci. 94: 5998-6000, 1997.
Aizawa, S.; Nakano, H.; Ishida, T.; Horie, R.; Nagai, M.; Ito, K.; Yagita, H.; Okumura, K.; Inoue, J.; Watanabe, T.: Tumor necrosis factor receptor-associated factor (TRAF) 5 and TRAF2 are involved in CD30-mediated NF-kappa-B activation. J. Biol. Chem. 272: 2042-2045, 1997.
Gregor, P.; Reeves, R. H.; Jabs, E. W.; Yang, X.; Dackowski, W.; Rochelle, J. M.; Brown, R. H., Jr.; Haines, J. L.; O'Hara, B. F.; Uhl, G. R.; Seldin, M. F.: Chromosomal localization of glutamate receptor genes: relationship to familial amyotrophic lateral sclerosis and other neurological disorders of mice and humans. Proc. Nat. Acad. Sci. 90: 3053-3057, 1993.
Sun, W.; Ferrer-Montiel, A. V.; Schinder, A. F.; McPherson, J. P.; Evans, G. A.; Montal, M.: Molecular cloning, chromosomal mapping, and functional expression of human brain glutamate receptors. Proc. Nat. Acad. Sci. 89: 1443-1447, 1992.
Anderson, L. A.; Friedman, L.; Osborne-Lawrence, S.; Lynch, E.; Weissenbach, J.; Bowcock, A.; King, M.-C.: High-density genetic map of the BRCA1 region of chromosome 17q12-q21. Genomics 17: 618-623, 1993
Zurawski, S. M.; Vega, F., Jr.; Huyghe, B.; Zurawski, G.: Receptors for interleukin-13 and interleukin-4 are complex and share a novel component that functions in signal transduction. EMBO J. 12: 2663-2670, 1993.
Aleman, A.; Verhaar, H. J. J.; De Haan, E. H. F.; De Vries, W. R.; Samson, M. M.; Drent, M. L.; Van Der Veen, E. A.; Koppeschaar, H. P. F.: Insulin-like growth factor-I and cognitive function in healthy older men. J. Clin. Endocr. Metab. 84: 471-475, 1999.
Baker, J.; Liu, J.-P.; Robertson, E. J.; Efstratiadis, A.: Role of insulin-like growth factors in embryonic and postnatal growth. Cell 75:73-82, 1993.
Bowcock, A.; Sartorelli, V.: Polymorphism and mapping of the IGF1 gene, and absence of association with stature among African Pygmies. Hum. Genet. 85: 349-354, 1990.
Camacho-Hubner, C.; Woods, K. A.; Miraki-Moud, F.; Hindmarsh, P. C.; Clark, A. J.; Hansson, Y.; Johnston, A.; Baxter, R. C.; Savage, M. O.: Effects of recombinant human insulin-like growth factor I (IGF-1) therapy on the growth hormone-IGF system of a patient with a partial IGF-I gene deletion. J. Clin. Endocr. Metab. 84: 1611-1616, 1999.
Goddard, A. D.; Covello, R.; Luoh, S.-M.; Clackson, T.; Attie, K. M.; Gesundheit, N.; Rundle, A. C.; Wells, J. A.; Carlsson, L. M. S.: Mutations of the growth hormone receptor in children with idiopathic short stature. New Eng. J. Med. 333: 1093-1098, 1995.
Guler, H.-P.; Binz, K.; Eigenmann, E.; Jaggi, S.; Zimmermann, D.; Zapf, J.; Froesch, E. R.: Small stature and insulin-like growth factors: prolonged treatment of mini-poodles with recombinant human insulin-like growth factor 1. Acta Endocr. 121: 456-464, 1989.
Garland, D.; Rao, P. V.; Del Corso, A.; Mura, U.; Zigler, J. S., Jr.: Zeta-crystallin is a major protein in the lens of Camelus dromedarius. Arch. Biochem. Biophys. 285: 134-136, 1991.
Gonzalez, P.; Rao, P. V.; Zigler, J. S., Jr.: Organization of the human zeta-crystallin/quinone reductase gene (CRYZ). Genomics 21:317-324, 1994.
Heinzmann, C.; Kojis, T. L.; Gonzalez, P.; Rao, P. V.; Zigler, J. S., Jr.; Polymeropoulos, M. H.; Klisak, I.; Sparkes, R. S.; Mohandas, T.; Bateman, J. B.: Assignment of the zeta-crystallin gene (CRYZ) to human chromosome 1p22-p31 and identification of restriction fragment-length polymorphisms. Genomics 23: 403-407, 1994.
Huang, Q.-L.; Russell, P.; Stone, S. H.; Zigler, J. S., Jr.: Zeta-crystallin, a novel lens protein from the guinea pig. Curr. Eye Res. 6: 725-732, 1987.
Rodriguez, I. R.; Gonzalez, P.; Zigler, J. S., Jr.; Borras, T.: A guinea-pig hereditary cataract contains a splice site deletion in a crystallin gene. Biochim. Biophys. Acta 1180: 44-52, 1992.
Kremmidiotis, G.; Baker, E.; Crawford, J.; Eyre, H. J.; Nahmias, J.; Callen, D. F.: Localization of human cadherin genes to chromosome regions exhibiting cancer-related loss of heterozygosity. Genomics 49:467-471, 1998.
Clayerie, J.-M.; Hardelin, J.-P.; Legouis, R.; Levilliers, J.; Bougueleret, L.; Mattei, M.-G.; Petit, C.: Characterization and chromosomal assignment of a human cDNA encoding a protein related to the murine 102-kDa cadherin-associated protein (alpha-catenin). Genomics 15:13-20, 1993.
Cook, S. A.; Bronson, R. T.; Donahue, L. R.; Ben-Arie, N.; Davisson, M. T.: Cerebellar deficient folia (cdf): a new mutation on mouse chromosome 6. Mammalian Genome 8: 108-112, 1997.
Herrenknecht, K.; Ozawa, M.; Eckerskorn, C.; Lottspeich, F.; Lenter, M.; Kemler, R.: The uvomorulin-anchorage protein alpha-catenin is a vinculin homologue. Proc. Nat. Acad. Sci. 88: 9156-9160, 1991.
Park, C.; Falls, W.; Finger, J. H.; Longo-Guess, C. M.; Ackerman, S. L.: Deletion in Catna2, encoding alpha-N-catenin, causes cerebellar and hippocampal lamination defects and impaired startle modulation. Nature Genet. 31: 279-284, 2002.
Hanson, I.; Churchill, A.; Love, J.; Axton, R.; Moore, T.; Clarke, M.; Meire, F.; van Heyningen, V.: Missense mutations in the most ancient residues of the PAX6 paired domain underlie a spectrum of human congenital eye malformations. Hum. Molec. Genet. 8: 165-172, 1999.
Martin-Subero, J. I.; Gesk, S.; Harder, L.; Sonoki, T.; Tucker, P. W.; Schlegelberger, B.; Grote, W.; Novo, F. J.; Calasanz, M. J.; Hansmann, M. L.; Dyer, M. J. S.; Siebert, R.: Recurrent involvement of the REL and BCL11A loci in classical Hodgkin lymphoma. Blood 99:1474-1477, 2002.
Hankinson, S. E.; Willett, W. C.; Colditz, G. A.; Hunter, D. J.; Michaud, D. S.; Deroo, B.; Rosner, B.; Speizer, F. E.; Pollak, M.: Circulating concentrations of insulin-like growth factor-I and risk of breast cancer. Lancet 351: 1393-1396, 1998.
Emi, M.; Asaoka, H.; Matsumoto, A.; Itakura, H.; Kurihara, Y.; Wada, Y.; Kanamori, H.; Yazaki, Y.; Takahashi, E.; Lepert, M.; Lalouel, J.-M.; Kodama, T.; Mukai, T.: Structure, organization, and chromosomal mapping of the human macrophage scavenger receptor gene. J. Biol. Chem. 268: 2120-2125, 1993.
Latil, A.; Lidereau, R.: Genetic aspects of prostate cancer. Virchows Arch. 432: 389-406, 1998.
Matsumoto, A.; Naito, M.; Itakura, H.; Ikemoto, S.; Asaoka, H.; Hayakawa, I.; Kanamori, H.; Aburatani, H.; Takaku, F.; Suzuki, H.; Kobari, Y.; Miyai, T.; Takahashi, K.; Cohen, E. H.; Wydro, R.; Housman, D. E.; Kodama, T.: Human macrophage scavenger receptors: primary structure, expression, and localization in atherosclerotic lesions. Proc. Nat. Acad. Sci. 87: 9133-9137, 1990.
Xu, J.; Zheng, S. L.; Hawkins, G. A.; Faith, D. A.; Kelly, B.; Isaacs, S. D.; Wiley, K. E.; Chang, B.; Ewing, C. M.; Bujnovszky, P.; Carpten, J. D.; Bleecker, E. R.; Walsh, P. C.; Trent, J. M.; Meyers, D. A.; Isaacs, W. B.: Linkage and association studies of prostate cancer susceptibility: evidence for linkage at 8p22-23. Am. J. Hum. Genet. 69: 341-350, 2001.
Xu, J.; Zheng, S. L.; Komiya, A.; Mychaleckyj, J. C.; Isaacs, S. D.; Hu, J. J.; Sterling, D.; Lange, E. M.; Hawkins, G. A.; Turner, A.; Ewing, C. M.; Faith, D. A.; and 19 others: Germline mutations and sequence variants of the macrophage scavenger receptor 1 gene are associated with prostate cancer risk. Nature Genet. 32: 321-325, 2002.
Harman, S. M.; Metter, E. J.; Blackman, M. R.; Landis, P. K.; Carter, H. B.: Serum levels of insulin-like growth factor I (IGF-I), IGF-II, IGF-binding protein-3, and prostate-specific antigen as predictors of clinical prostate cancer. J. Clin. Endocr. Metab. 85: 4258-4265, 2000.
Holly, J.: Insulin-like growth factor-I and new opportunities for cancer prevention. Lancet 351: 1373-1375, 1998.
Hoppener, J. W. M.; de Pagter-Holthuizen, P.; Geurts van Kessel, A. H. M.; Jansen, M.; Kittur, S. D.; Antonarakis, S. E.; Lips, C. J. M.; Sussenbach, J. S.: The human gene encoding insulin-like growth factor I is located on chromosome 12. Hum. Genet. 69: 157-160, 1985.
Le Bouc, Y.; Dreyer, D.; Jaeger, F.; Binoux, M.; Sondermeyer, P.: Complete characterization of the human IGF-I nucleotide sequence isolated from a newly constructed adult liver cDNA library. FEBS Lett. 196: 108-112, 1986.
Hellstrom, A.; Perruzzi, C.; Ju, M.; Engstrom, E.; Hard, A.-L.; Liu, J.-L.; Albertsson-Wikland, K.; Carlsson, B.; Niklasson, A.; Sjodell, L.; LeRoith, D.; Senger, D. R.; Smith, L. E. H.: Low IGF-I suppresses VEGF-survival signaling in retinal endothelial cells: direct correlation with clinical retinopathy of prematurity. Proc. Nat. Acad. Sci. 98:5804-5808, 2001.
Lembo, G.; Rockman, H. A.; Hunter, J. J.; Steinmetz, H.; Koch, W. J.; Ma, L.; Printz, M. P.; Ross, J., Jr.; Chien, K. R.; Powell-Braxton, L.: Elevated blood pressure and enhanced myocardial contractility in mice with severe IGF-1 deficiency. J. Clin. Invest. 98: 2648-2655, 1996.
Li, C. H.; Yamashiro, D.; Gospodarowicz, D.; Kaplan, S. L.; VanVliet, G.: Total synthesis of insulin-like growth factor I (somatomedin C). Proc. Nat. Acad. Sci. 80: 2216-2220, 1983.
Liu, J.-P.; Baker, J.; Perkins, A. S.; Robertson, E. J.; Efstratiadis, A.: Mice carrying null mutations of the genes encoding insulin-like growth factor I (Igf-1) and type 1 IGF receptor (Igf1r). Cell 75:59-72, 1993.
Mathews, L. S.; Norstedt, G.; Palmiter, R. D.: Regulation of insulin-like growth factor I gene expression by growth hormone. Proc. Nat. Acad. Sci. 83: 9343-9347, 1986.
Morton, C.; Rall, L.; Bell, G.; Shows, T.: Human insulin like growth factor-1 (IGF1) is encoded at 12q22-q24.1, and insulin-like growth factor-2 (IGF2) is at 11p15. (Abstract) Cytogenet. Cell Genet. 40:703 only, 1985.
Musaro, A.; McCullagh, K.; Paul, A.; Houghton, L.; Dobrowolny, G.; Molinaro, M.; Barton, E. R.; Sweeney, H. L.; Rosenthal, N.: Localized Igf-1 trans gene expression sustains hypertrophy and regeneration in senescent skeletal muscle. Nature Genet. 27: 195-200, 2001.
Musaro, A.; McCullagh, K. J. A.; Naya, F. J.; Olson, E. N.; Rosenthal, N.: IGF-1 induces skeletal myocyte hypertrophy through calcineurin in association with GATA-2 and NFATc1. Nature 400: 581-585, 1999.
Playford, M. P.; Bicknell, D.; Bodmer, W. F.; Macaulay, V. M.: Insulin-like growth factor 1 regulates the location, stability, and transcriptional activity of beta-catenin. Proc. Nat. Acad. Sci. 97:12103-12108, 2000.
Powell-Braxton, L.; Hollingshead, P.; Warburton, C.; Dowd, M.; Pitts-Meek, S.; Dalton, D.; Gillett, N.; Stewart, T. A.: IGF-I is required for normal embryonic growth in mice. Genes Dev. 7: 2609-2617, 1993.
Rapp, R.; Deger, A.; Blum, W.; Koch, R.; Weber, U.: Characterization of the protein which binds insulin-like growth factor in human serum. Europ. J. Biochem. 172: 421-425, 1988.
Rotwein, P.: Two insulin-like growth factor I messenger RNAs are expressed in human liver. Proc. Nat. Acad. Sci. 83: 77-81, 1986.
Schoenle, E. J.; Zenobi, P. D.; Torresani, T.; Werder, E. A.; Zachmann, M.; Froesch, E. R.: Recombinant human insulin-like growth factor I (rhIGF I) reduces hyperglycaemia in patients with extreme insulin resistance. Diabetologia 34: 675-679, 1991.
Semsarian, C.; Wu, M.-J.; Ju, Y.-K.; Marciniec, T.; Yeoh, T.; Allen, D. G.; Harvey, R. P.; Graham, R. M.: Skeletal muscle hypertrophy is mediated by a Ca(2+)-dependent calcineurin signalling pathway. Nature 400:576-581, 1999.
Svoboda, M. E.; Van Wyk, J. J.; Klapper, D. G.; Fellows, R. E.; Grissom, F. E.; Schleuter, R. J.: Purification of somatomedin-C from human plasma: chemical and biological properties, partial sequence analysis, and relationship to other somatomedins. Biochemistry 19:790-797, 1980.
Taylor, B. A.; Grieco, D.: Localization of the gene encoding insulin-like growth factor I on mouse chromosome 10. Cytogenet. Cell Genet. 56: 57-58, 1991.
Tricoli, J. V.; Rall, L. B.; Scott, J.; Bell, G. I.; Shows, T. B.: Insulin-like growth factor genes: chromosome organization and association with disease. (Abstract) Am. J. Hum. Genet. 36: 121Sonly, 1984.
Tricoli, J. V.; Rall, L. B.; Scott, J.; Bell, G. I.; Shows, T. B.: Localization of insulin-like growth factor genes to human chromosomes 11 and 12. Nature 310: 784-786, 1984
Ullrich, A.; Berman, C. H.; Dull, T. J.; Gray, A.; Lee, J. M.: Isolation of the human insulin-like growth factor I gene using a single synthetic DNA probe. EMBO J. 3: 361-364, 1984.
Thomas, J. T.; Kilpatrick, M. W.; Lin, K.; Erlacher, L.; Lembessis, P.; Costa, T.; Tsipouras, P.; Luyten, F. P.: Disruption of human limb morphogenesis by a dominant negative mutation in CDMP1. Nature Genet. 58-64, 1997.
Fryns, J. P.; van den Berghe, K.; van Assche, A.; van den Berghe, H.: Prenatal diagnosis of campomelic dwarfism. Clin. Genet. 19:199-201, 1981.
Mellows, H. J.; Pryse-Davies, J.; Bennett, M. J.; Carter, C. O.: The camptomelic syndrome in two female siblings. Clin. Genet. 18:137-141, 1980.
Xu, L.; Xia, J.; Jiang, H.; Zhou, J.; Li, H.; Wang, D.; Pan, Q.; Long, Z.; Fan, C.; Deng, H.-X.: Mutation analysis of hereditary multiple exostoses in the Chinese. Hum. Genet. 105: 45-50, 1999.
Simmons, A. D.; Musy, M. M.; Lopes, C. S.; Hwang, L.-Y.; Yang, Y.-P.; Lovett, M.: A direct interaction between EXT proteins and glycosyltransferases is defective in hereditary multiple exostoses. Hum. Molec. Genet. 8: 2155-2164, 1999
Bevilacqua, M.; Butcher, E.; Furie, B.; Furie, B.; Gallatin, M.; Gimbrone, M.; Harlan, J.; Kishimoto, K.; Lasky, L.; McEver, R.; Paulson, J.; Rosen, S.; Seed, B.; Siegelman, M.; Springer, T.; Stoolman, L.; Tedder, T.; Varki, A.; Wagner, D.; Weissman, I.; Zimmerman, G.: Selectins: a family of adhesion receptors. (Letter) Cell 67: 233 only, 1991.
Bevilacqua, M. P.; Stengelin, S.; Gimbrone, M. A., Jr.; Seed, B.: Endothelial leukocyte adhesion molecule 1: an inducible receptor for neutrophils related to complement regulatory proteins and lectins. Science 243:1160-1165, 1989.
Collins, T.; Williams, A.; Johnston, G. I.; Kim, J.; Eddy, R.; Shows, T.; Gimbrone, M. A., Jr.; Bevilacqua, M. P.: Structure and chromosomal location of the gene for endothelial-leukocyte adhesion molecule 1. J. Biol. Chem. 266: 2466-2473, 1991.
DeLisser, H. M.; Christofidou-Solomidou, M.; Sun, J.; Nakada, M. T.; Sullivan, K. E.: Loss of endothelial surface expression of E-selectin in a patient with recurrent infections. Blood 94: 884-894, 1999.
Wang, N.; Chintala, S. K.; Fini, M. E.; Sc human, J. S.: Activation of a tissue-specific stress response in the aqueous outflow pathway of the eye defines the glaucoma disease phenotype. Nature Med. 7:304-309, 2001.
Watson, M. L.; Kingsmore, S. F.; Johnston, G. I.; Siegelman, M. H.; Le Beau, M. M.; Lemons, R. S.; Bora, N. S.; Howard, T. A.; Weissman, I. L.; McEver, R. P.; Seldin, M. F.: Genomic organization of the selectin family of leukocyte adhesion molecules on human and mouse chromosome 1. J. Exp. Med. 172: 263-272, 1990.
Wenzel, K.; Felix, S.; Kleber, F. X.; Brachold, R.; Menke, T.; Schattke, S.; Schulte, K. L.; Glaser, C.; Rohde, K.; Baumann, G.; Speer, A.: E-selectin polymorphism and atherosclerosis: an association study. Hum. Molec. Genet. 3: 1935-1937, 1994.
Zheng, F.; Chevalier, J. A.; Zhang, L. Q.; Virgil, D.; Ye, S. Q.; Kwiterovich, P. O.: An Hphl polymorphism in the Eselectin gene is associated with premature coronary artery disease. Clin. Genet. 59:58-64, 2001.
Eckenstein, F. P.: Fibroblast growth factors in the nervous system. J. Neurobiol. 25: 1467-1480, 1994.
Jung, J.; Zheng, M.; Goldfarb, M.; Zaret, K. S.: Initiation of-mammalian liver development from endoderm by fibroblast growth factors. Science 284:1998-2003, 1999.
Mergia, A.; Eddy, R.; Abraham, J. A.; Fiddes, J. C.; Shows, T. B.: The genes for basic and acidic fibroblast growth factors areon different human chromosomes. Biochem. Biophys. Res. Commun. 138:644-651, 1986.
Plotnikov, A. N.; Hubbard, S. R.; Schlessinger, J.; Mohammadi, M.: Crystal structures of two FGF-FGFR complexes reveal the determinants of ligand-receptor specificity. Cell 101: 413-424, 2000.
Willecke, K.; Jungbluth, S.; Dahl, E.; Hennemann, H.; Heynkes, R.; Grzeschik, K.-H.: Six genes of the human connexin gene family coding for gap junctional proteins are assigned to four different human chromosomes. Europ. J. Cell Biol. 53: 275-280, 1990.
Oviedo-Orta, E.; Hoy, T.; Evans, W. H.: Intercellular communication in the immune system: differential expression of connexin 40 and 43, and perturbation of gap junction channel functions in peripheral blood and tonsil human lymphocyte subpopulations. Immunology 99: 578-590, 2000.
Britz-Cunningham, S. H.; Shah, M. M.; Zuppan, C. W.; Fletcher, W. H.: Mutations of the connexin 43 gap-junction gene in patients with heart malformations and defects of laterality. New Eng. J. Med. 332:1323-1329, 1995.
Brueckner, M.; D'Eustachio, P.; Horwich, A. L.: Linkage mapping of a mouse gene, iv, that controls left-right asymmetry of the heart and viscera. Proc. Nat. Acad. Sci. 86: 5035-5038, 1989.
Burdine, R. D.; Schier, A. F.: Conserved and divergent mechanisms in left-right axis formation. Genes Dev. 14: 763-776, 2000.
Casey, B.; Ballabio, A.: Connexin 43 mutations in sporadic and familial defects of laterality. (Letter) New Eng. J. Med. 333: 941, 1995.
Corcos, I. A.; Meese, E. U.; Loch-Caruso, R.: Human connexin 43 gene locus, GJA1, sublocalized to band 6q21-q23.2. Cytogenet. Cell Genet. 64: 31-32, 1993.
Debrus, S.; Tuffery, S.; Matsuoka, R.; Galal, O.; Sarda, P.; Sauer, U.; Bozio, A.; Tanman, B.; Toutain, A.; Claustres, M.; Le Paslier, D.; Bouvagnet, P.: Lack of evidence for connexin 43 gene mutations in human autosomal recessive lateralization defects. J. Molec. Cell. Cardiol. 29: 1423-1431, 1997.
Wijnen, J. T.; Oldenburg, M.; Bloemendal, H.; Meera Khan, P.: GS(gamma-S)-crystallin (CRYGS) assignment to chromosome 3. (Abstract) Cytogenet. Cell Genet. 51: 1108 only, 1989.
Chen, L. Z.; Harris, P. C.; Apostolou, S.; Baker, E.; Holman, K.; Lane, S. A.; Nancarrow, J. K.; Whitmore, S. A.; Stallings, R. L.; Hildebrand, C. E.; Richards, R. I.; Sutherland, G. R.; Callen, D. F.: A refined physical map of the long arm of human chromosome 16. Genomics 10:308-312, 1991.
Albrecht, F. E.; Drago, J.; Felder, R. A.; Printz, M. P.; Eisner, G. M.; Robillard, J. E.; Sibley, D. R.; Westphal, H. J.; Jose, P. A.: Role of the D-1A dopamine receptor in the pathogenesis of gene tichypertension. J. Clin. Invest. 97: 2283-2288, 1996.
Bermak, J. C.; Li, M.; Bullock, C.; Zhou, Q.-Y.: Regulation of transport of the dopamine D1 receptor by a new membrane-associated ER protein. Nature Cell Biol. 3: 492-498, 2001.
Boultwood, J.; Lewis, M. S.; Wainscoat, J. S.: Physical linkage of glucocorticoid receptor (GRL) and dopamine D1 receptor (DRD1) on the long arm of chromosome 5. (Abstract) Cytogenet. Cell Genet. 58:1894 only, 1991.
Castner, S. A.; Williams, G. V.; Goldman-Rakic, P. S.: Reversal of antipsychotic-induced working memory deficits by short-term dopamine D1 receptor stimulation. Science 287: 2020-2022, 2000.
Dearry, A.; Gingrich, J. A.; Falardeau, P.; Fremeau, R. T., Jr.; Bates, M. D.; Caron, M. G.: Molecular cloning and expression of the gene for a human D(1) dopamine receptor. Nature 347: 72-76, 1990.
Grandy, D. K.; Zhou, Q.-Y.; Allen, L.; Litt, R.; Magenis, R. E.; Civelli, O.; Litt, M.: A human D(1) dopamine receptor gene is located on chromosome 5 at q35.1 and identifies an EcoRI RFLP. Am. J. Hum. Genet. 47: 828-834, 1990.
Krushkal, J.; Xiong, M.; Ferrell, R.; Sing, C. F.; Turner, S. T.; Boerwinkle, E.: Linkage and association of adrenergic and dopamine receptor genes in the distal portion of the long arm of chromosome S with systolic blood pressure variation. Hum. Molec. Genet. 7:1379-1383, 1998.
Mayerhofer, A.; Hemmings, H. C., Jr.; Snyder, G. L.; Greengard, P.; Boddien, S.; Berg, U.; Brucker, C.: Functional dopamine-1 receptors and DARPP-32 are expressed in human ovary and granulosa luteal cells in vitro. J. Clin. Endocr. Metab. 84: 257-264, 1999.
Sunahara, R. K.; Niznik, H. B.; Weiner, D. M.; Stormann, T. M.; Brann, M. R.; Kennedy, J. L.; Gelernter, J. E.; Rozmahel, R.; Yang, Y.; Israel, Y.; Seeman, P.; O'Dowd, B. F.: Human dopamine D1 receptor encoded by an intronless gene on chromosome 5. Nature 347: 80-83, 1990.
Wilkie, T. M.; Chen, Y.; Gilbert, D. J.; Moore, K. J.; Yu, L.; Simon, M. I.; Copeland, N. G.; Jenkins, N. A.: Identification, chromosomal location, and genome organization of mammalian G-protein-coupled receptors. Genomics 18:175-184, 1993.
Xu, M.; Moratalla, R.; Gold, L. H.; Hiroi, N.; Koob, G. F.; Graybiel, A. M.; Tonegawa, S.: Dopamine D1 receptor mutant mice are deficient in striatal expression of dynorphin and in dopamine-mediated behavioral responses. Cell 79: 729-742, 1994.
Zhou, Q.-Y.; Grandy, D. K.; Thambi, L.; Kushner, J. A.; Van Tol, H. H. M.; Cone, R.; Pribnow, D.; Salon, J.; Bunzow, J. R.; Civelli, O.: Cloning and expression of human and rat D(1) dopamine receptors. Nature 347:76-80, 1990.
Fan, Y.-S.; Eddy, R. L.; Byers, M. G.; Haley, L. L.; Henry, W. M.; Kayano, T.; Shows, T. B.; Bell, G. I.: Assignment of genes encoding three human glucose transporter/transporter-like proteins (GLUT4, GLUT5 and GLUT6) to chromosomes 17, 1 and 5, respectively. (Abstract) Cytogenet. Cell Genet. 51: 997 only, 1989.
Aitken, D. A.; Ferguson-Smith, M. A.: Gene dosage evidence for the regional assignment of the GOT-S structural gene locus to 10q24-10q25. Cytogenet. Cell Genet. 22: 468-471, 1978.
Creagan, R.; Tischfield, J.; McMorris, F. A.; Chen, S.-H.; Hirschi, M.; Chen, T.-T.; Ricciuti, F.; Ruddle, F. H.: Assignment of the genes for human peptidase A to chromosome 18 and cytoplasmic glutamic oxaloacetate transaminase to chromosome 10 using somatic-cell hybrids. Cytogenet. Cell Genet. 12: 187-198, 1973.
Ford, G. C.; Eichele, G.; Jansonius, J. N.: Three-dimensional structure of a pyridoxal-phosphate-dependent enzyme, mitochondrial aspartate aminotransferase. Proc. Nat. Acad. Sci. 77: 2559-2563, 1980.
Gitelman, B. J.; Tomkins, D. J.; Partington, M. W.; Roberts, M. H.; Simpson, N. E.: Gene dosage studies of glutamic oxaloacetic transaminase (GOT) and hexo kinase (HK) in two patients with possible partial trisomy 10q. (Abstract) Am. J. Hum. Genet. 32: 41A only, 1980.
Junien, C.; Despoisse, S.; Turleau, C.; de Grouchy, J.; Bucher, T.; Fundele, R.: Assignment of phosphoglycerate mutase (PGAMA) to human chromosome 10: regional mapping of GOT1 and PGAMA to subbands 10q26.1 (or q25.3). Ann. Genet. 25: 25-27, 1982.
Overhauser, J.; Mewar, R.; Rojas, K.; Lia, K.; Kline, A. D.; Silverman, G. A.: STS map of genes and anonymous DNA fragments on human chromosome 18 using a panel of somatic cell hybrids. Genomics 15: 387-391, 1993.
Koch, G.; Lalley, P. A.; McAvoy, M.; Shows, T. B.: Assignment of LIPA, associated with human acid lipase deficiency, to human chromosome 10 and comparative assignment to mouse chromosome 19. Somat. Cell Genet. 7: 345-358, 1981.
Asada, H.; Kawamura, Y.; Maruyama, K.; Kume, H.; Ding, R.-G.; Ji, F. Y.; Kanbara, N.; Kuzume, H.; Sanbo, M.; Yagi, T.; Obata, K.: Mice lacking the 65 kDa isoform of glutamic acid decarboxylase (GAD65) maintain normal levels of GAD67 and GABA in their brains but are susceptible to seizures. Biochem. Biophys. Res. Commun. 229: 891-895, 1996.
Bu, D.-F.; Erlander, M. G.; Hitz, B. C.; Tillakaratne, N. J. K.; Kaufman, D. L.; Wagner-McPherson, C. B.; Evans, G. A.; Tobin, A. J.: Two human glutamate decarboxylases, 65-kDa GAD and 67-kDa GAD, are each encoded by a single gene. Proc. Nat. Acad. Sci. 89: 2115-2119, 1992.
Edelhoff, S.; Grubin, C. E.; Karlsen, A. E.; Adler, D. A.; Foster, D.; Disteche, C. M.; Lernmark, A.: Mapping of glutamic acid decarboxylase (GAD) genes. Genomics 17: 93-97, 1993.
McKusick, V. A.: The morbid anatomy of the human genome: a review of gene mapping in clinical medicine (part 1). Medicine 65: 1-33, 1986
Panteghini, M.: Aspartate aminotransferase isoenzymes. Clin. Biochem. 23: 311-319, 1990.
Pol, S.; Bousquet-Lernercier, B.; Pave-Preux, M.; Bulle, F.; Passage, E.; Hanoune, J.; Mattei, M. G.; Barouki, R.: Chromosomal localization of human aspartate aminotransferase genes by in situ hybridization. Hum. Genet. 83: 159-164, 1989.
Scott, E. M.; Wright, R. C.: An alternate method for demonstration of erythrocytic amino transferases on starch gels. Am. J. Hum. Genet. 33:561-563, 1981.
Spritz, R. A.; Emanuel, B. S.; Chern, C. J.; Mellman, W. J.: Gene dosage effect: intraband mapping of human soluble glutamic oxaloacetic transaminase. Cytogenet. Cell Genet. 23: 149-156, 1979.
Tomkins, D. J.; Gitelman, B. J.; Roberts, M. H.: Confirmation of a de novo duplication, dup(10)(q24-q26), by GOT1 gene dosage studies. Hum. Genet. 63: 369-373, 1983.
Wang, C.-Y.; Huang, Y.-Q.; Shi, J.-O.; Marron, M. P.; Ruan, Q.-G.; Hawkins-Lee, B.; Ochoa, B.; She, J.-X.: Genetic homogeneity, high-resolution mapping, and mutation analysis of the urofacial (Ochoa) syndrome and exclusion of the glutamate oxaloacetate transaminase gene (GOT1) in the critical region as the disease gene. Am. J. Med. Genet. 84:454-459, 1999.
Wurzinger, K. H.; Mohrenweiser, H. W.: Studies on the genetic and non-genetic (physiological) variation of human erythrocyte glutamic oxaloacetic transaminase. Ann. Hum. Genet. 46: 191-201, 1982.
Abel, E. D.; Kaulbach, H. C.; Tian, R.; Hopkins, J. C. A.; Duffy, J.; Doetschman, T.; Minnemann, T.; Boers, M.-E.; Hadro, E.; Oberste-Berghaus, C.; Quist, W.; Lowell, B. B.; Ingwall, J. S.; Kahn, B. B.: Cardiachypertrophy with preserved contractile function after selective deletion of GLUT4 from the heart. J. Clin. Invest. 104: 1703-1714, 1999.
Abel, E. D.; Peroni, O.; Kim, J. K.; Kim, Y.-B.; Boss, O.; Hadro, E.; Minnemann, T.; Shulman, G. I.; Kahn, B. B.: Adipose-selective targeting of the GLUT4 gene impairs insulin action in muscle and liver. Nature 409:729-733, 2001.
Bell, G. I.; Kayano, T.; Buse, J. B.; Burant, C. F.; Takeda, J.; Lin, D.; Fukumoto, H.; Seino, S.: Molecular biology of mammalian glucose transporters. Diabetes Care 13: 198-208, 1990.
Bell, G. I.; Murray, J. C.; Nakamura, Y.; Kayano, T.; Eddy, R. L.; Fan, Y.-S.; Byers, M. G.; Shows, T. B.: Polymorphic human insulin-responsive glucose-transporter gene on chromosome 17p13. Diabetes 38: 1072-1075, 1989.
Birnbaum, M. J.: Identification of a novel gene encoding an insulin-responsive glucose transporter protein. Cell 57: 305-315, 1989.
Chiang, S.-H.; Baumann, C. A.; Kanzaki, M.; Thurmond, D. C.; Watson, R. T.; Neudauer, C. L.; Macara, I. G.; Pessin, J. E.; Saltiel, A. R.: Insulin-stimulated GLUT4 translocation requires the CAP-dependent activation of TC10. Nature 410: 944-948, 2001.
Garvey, W. T.; Maianu, L.; Zhu, J.-H.; Brechtel-Hook, G.; Wallace, P.; Baron, A. D.: Evidence for defects in the trafficking and translocation of GLUT4 glucose transporters in skeletal muscle as a cause of human insulin resistance. J. Clin. Invest. 101: 2377-2386, 1998.
Ikemoto, S.; Thompson, K. S.; Itakura, H.; Lane, M. D.; Ezaki, O.: Expression of an insulin-responsive glucose transporter (GLUT4) mini gene in transgenic mice: effect of exercise and role in glucose homeostasis. Proc. Nat. Acad. Sci. 92: 865-869, 1995.
Katz, E. B.; Stenbit, A. E.; Hatton, K.; DePinho, R.; Charron, M. J.: Cardiac and adipose tissue abnormalities but not diabetes in mice deficient in GLUT4. Nature 377: 151-155, 1995
Kusari, J.; Verma, U. S.; Buse, J. B.; Henry, R. R.; Olefsky, J. M.: Analysis of the gene sequences of the insulin receptor and the insulin-sensitive glucose transporter (GLUT-4) in patients with common-type non-insulin-dependent diabetes mellitus. J. Clin. Invest. 88:1323-1330, 1991.
Muraoka, A.; Sakura, H.; Kim, K.; Kishimoto, M.; Akanuma, Y.; Buse, J. B.; Yasuda, K.; Seino, S.; Bell, G. I.; Yazaki, Y.; Kasuga, M.; Kadowaki, T.: Polymorphism in exon 4a of the human GLUT4/muscle-fat facilitative glucose transporter gene detected by SSCP. Nucleic Acids Res. 19: 4313 only, 1991.
Ribon, V.; Printen, J. A.; Hoffman, N. G.; Kay, B. K.; Saltiel, A. R.: A novel, multifunctional c-Cbl binding protein in insulin receptor signaling in 3T3-L1 adipocytes. Molec. Cell. Biol. 18:872-879, 1998.
Zisman, A.; Peroni, O. D.; Abel, E. D.; Michael, M. D.; Mauvais-Jarvis, F.; Lowell, B. B.; Wojtaszewski, J. F. P.; Hirshman, M. F.; Virkamaki, A.; Goodyear, L. J.; Kahn, C. R.; Kahn, B. B.: Targeted disruption of the glucose transporter 4 selectively in muscle causes insulin resistance and glucose intolerance. Nature Med. 6: 924-928, 2000.
Fishman, G. I.; Eddy, R. L.; Shows, T. B.; Rosenthal, L.; Leinwand, L. A.: The human connexin gene family of gap junction proteins: distinct chromosomal locations but similar structures. Genomics 10: 250-256, 1991.
Fukushige, S.; Murotsu, T.; Matsubara, K.: Chromosomal assignment of human genes for gastrin, thyrotropin (TSH)-beta subunit and C-erb-2 by chromosome sorting combined with velocity sedimentation and southern hybridization. Biochem. Biophys. Res. Commun. 134: 477-483, 1986.
Justice, M. J.; Gilbert, D. J.; Kinzler, K. W.; Vogelstein, B.; Buchberg, A. M.; Ceci, J. D.; Matsuda, Y.; Chapman, V. M.; Patriotis, C.; Makris, A.; Tsichlis, P. N.; Jenkins, N. A.; Copeland, N. G.: A molecular genetic linkage map of mouse chromosome 18 reveals extensive linkage conservation with human chromosomes 5 and 18. Genomics 13:1281-1288, 1992.
Triantafilou, K.; Triantafilou, M.; Dedrick, R. L.: A CD14-independent LPS receptor cluster. Nature Immun. 2: 338-345, 2001.
Lamaze, C.; Dujeancourt, A.; Baba, T.; Lo, C. G.; Benmerah, A.; Dautry-Varsat, A.: Interleukin 2 receptors and detergent-resistant membrane domains define a clathrin-independent endocytic pathway. Molec. Cell 7: 661-671, 2001
Eklund, L.; Piuhola, J.; Komulainen, J.; Sormunen, R.; Ongvarrasopone, C.; Fassler, R.; Muona, A.; Iives, M.; Ruskoaho, H.; Takala, T. E. S.; Pihlajaniemi, T.: Lack of type XV collagen causes a skeletal myopathy and cardiovascular defects in mice. Proc. Nat. Acad. Sci. 98:1194-1199, 2001.
Hagg, P. M.; Hagg, P. O.; Peltonen, S.; Autio-Harmainen, H.; Pihlajaniemi, T.: Location of type XV collagen in human tissues and its accumulation in the interstitial matrix of the fibrotic kidney. Am. J. Path. 150:2075-2086, 1997.
Hagg, P. M.; Horelli-Kuitunen, N.; Eklund, L.; Palotie, A.; Pihlajaniemi, T.: Cloning of mouse type XV collagen sequences and mapping of the corresponding gene to 4B1-3: comparison of mouse and human alpha-1(XV) collagen sequences indicates divergence in the number of small collagenous domains. Genomics 45: 31-41, 1997.
Hagg, P. M.; Muona, A.; Lietard, J.; Kivirikko, S.; Pihlajaniemi, T.: Complete exon-intron organization of the human gene for the alpha-1 chain of type XV collagen (COL15A1) and comparison with the homologous Col18a1 gene. J. Biol. Chem. 273: 17824-17831, 1998.
Huebner, K.; Cannizzaro, L. A.; Jabs, E. W.; Kivirikko, S.; Manzone, H.; Pihlajaniemi, T.; Myers, J. C.: Chromosomal assignment of a gene encoding a new collagen type (COL15A1) to 9q21-q22. Genomics 14:220-224, 1992.
Kivirikko, S.; Heinamaki, P.; Rehn, M.; Honkanen, N.; Myers, J. C.; Pihlajaniemi, T.: Primary structure of the alpha-1 chain of human type XV collagen and exon-intron organization in the 3-prime region of the corresponding gene. J. Biol. Chem. 269: 4773-4779, 1994.
Muragaki, Y.; Abe, N.; Ninomiya, Y.; Olsen, B. R.; Ooshima, A.: The human alpha-1(XV) collagen chain contains a large amino-terminal non-triple helical domain with a tandem repeat structure and homology to alpha-1(XVIII) collagen. J. Biol. Chem. 269: 4042-4046, 1994.
Myers, J. C.; Dion, A. S.; Abraham, V.; Amenta, P. S.: Type XV collagen exhibits a widespread distribution in human tissues but a distinct localization in basement membrane zones. Cell Tissue Res. 286:493-505, 1996.
Myers, J. C.; Kivirikko, S.; Gordon, M. K.; Dion, A. S.; Pihlajaniemi, T.: Identification of a previously unknown human collagen chain, alpha-1(XV), characterized by extensive interruptions in the triple-helical region. Proc. Nat. Acad. Sci. 89: 10144-10148, 1992.
Ramchandran, R.; Dhanabal, M.; Volk, R.; Waterman, M. J. F.; Segal, M.; Lu, H.; Knebelmann, B.; Sukhatme, V. P.: Antiangiogenic activity of restin, NC10 domain of human collagen XV: comparison to endostatin. Biochem. Biophys. Res. Commun. 255: 735-739, 1999.
Rehn, M.; Hintikka, E.; Pihlajaniemi, T.: Primary structure of the alpha 1 chain of mouse type XVIII collagen, partial structure of the corresponding gene, and comparison of the alpha 1(XVIII) chain with its homologue, the alpha 1(XV) collagen chain. J. Biol. Chem. 269:13929-13935, 1994.
Sasaki, T.; Larsson, H.; Tisi, D.; Claesson-Welsh, L.; Hohenester, E.; Timpl, R.: Endostatins derived from collagens XV and XVIII differ in structural and binding properties, tissue distribution and anti-angiogenic activity. J. Molec. Biol. 301: 1179-1190, 2000.
Hulsebos, T. J. M.; Jenkins, N. A.; Gilbert, D. J.; Copeland, N. G.: The beta crystallin genes on human chromosome 22 define a new region of homology with mouse chromosome 5. Genomics 25: 574-576, 1995.
Lampi, K. J.; Ma, Z.; Shih, M.; Shearer, T. R.; Smith, J. B.; Smith, D. L.; David, L. L.: Sequence analysis of beta-A3, beta-B3, and beta-A4 crystallins completes the identification of the major proteins in young human lens. J. Biol. Chem. 272: 2268-2275, 1997.
van Rens, G.; Geurts van Kessel, A.; Bloemendal, H.: Localization of the beta-A4 crystallin (CRYBA4) gene on human chromosome 22 in the region q11.2-q13.1. (Abstract) Cytogenet. Cell Genet. 58: 2052 only, 1991.
van Rens, G. L. M.; Geurts van Kessel, A. H. M.; Bloemendal, H.: Localization of the beta-A4-crystallin gene (CRYBA4) on human chromosome 22 in the region q11.2-q13.1. Cytogenet. Cell Genet. 61: 180-183, 1992.
den Dunnen, J. T.; Jongbloed, R. J. E.; Geurts van Kessel, A. H. M.; Schoenmakers, J. G. G.: Human lens gamma-crystallin sequences are located in the p12-qter region of chromosome 2. Hum. Genet. 70:217-221, 1985.
Fajans, S. S.; Bell, G. I.; Polonsky, K. S.: Molecular mechanisms and clinical pathophysiology of maturity-onset diabetes of the young. New Eng. J. Med. 345: 971-980, 2001.
Wu, W. J.; Erickson, J. W.; Lin, R.; Cerione, R. A.: The gamma-subunit of the coatomer complex binds Cdc42 to mediate transformation. Nature 405:800-804, 2000.
Wunderle, V. M.; Critcher, R.; Hastie, N.; Goodfellow, P. N.; Schedl, A.: Deletion of long-range regulatory elements upstream of SOX9 causes campomelic dysplasia. Proc. Nat. Acad. Sci. 95: 10649-10654, 1998.
Young, I. D.; Zuccollo, J. M.; Maltby, E. L.; Broderick, N. J.: Campomelic dysplasia associated with a de novo 2q; 17q reciprocal translocation. J. Med. Genet. 29: 251-252, 1992.
Bard, L. A.: Heterogeneity in Waardenburg's syndrome: report of a family with ocular albinism. Arch. Ophthal. 96: 1193-1198, 1978.
Morell, R.; Spritz, R. A.; Ho, L.; Pierpont, J.; Guo, W.; Friedman, T. B.; Asher, J. H., Jr.: Apparent digenic inheritance of Waardenburg syndrome type 2 (WS2) and autosomal recessive ocular albinism (AROA). Hum. Molec. Genet. 6: 659-664, 1997.
Smith, S. D.; Kelley, P. M.; Kenyon, J. B.; Hoover, D.: Tietz syndrome (hypopigmentation/deafness) caused by mutation of MITF. J. Med. Genet. 37: 446-448, 2000.
Tietz, W.: A syndrome of deaf-mutism associated with albinism showing dominant autosomal inheritance. Am. J. Hum. Genet. 15: 259-264, 1963.
Hsieh, C.-L.; Kumar, N. M.; Gilula, N. B.; Francke, U.: Distribution of genes for gap junction membrane channel proteins on human and mouse chromosomes. Somat. Cell Molec. Genet. 17: 191-200, 1991.
DeStefano, A. L.; Baldwin, C. T.; Burzstyn, M.; Gavras, I.; Handy, D. E.; Joost, O.; Martel, T.; Nicolaou, M.; Schwartz, F.; Streeten, D. H. P.; Farrer, L. A.; Gavras, H.: Autosomal dominant orthostatichy potensive disorder maps to chromosome 18q. Am. J. Hum. Genet. 63:1425-1430, 1998.
Barnett, T.; Zimmermann, W.: Workshop report: proposed nomenclature for the carcinoembryonic antigen (CEA) gene family. Tumor Biol. 11:59-63, 1990.
Boulton, I. C.; Gray-Owen, S. D.: Neisserial binding to CEACAM1 arrests the activation and proliferation of CD4+ T lymphocytes. Nature Immun. 3: 229-236, 2002.
Ergun, S.; Kilic, N.; Ziegeler, G.; Hansen, A.; Nollau, P.; Gotze, J.; Wurmbach, J.-H.; Horst, A.; Weil, J.; Fernando, M.; Wagener, C.: CEA-related cell adhesion molecule 1: a potent angiogenic factor and a major effector of vascular endothelial growth factor. Molec. Cell 5: 311-320, 2000.
Hinoda, Y.; Neumaier, M.; Hefta, S. A.; Drzeniek, Z.; Wagener, C.; Shively, L.; Hefta, L. J. F.; Shively, J. E.; Paxton, R. J.: Molecular cloning of a cDNA coding biliary glyco protein I: primary structure of a glyco protein immunologically cross reactive with carcinoembryonic antigen. Proc. Nat. Acad. Sci. 85: 6959-6963, 1988.
Neumaier, M.; Paululat, S.; Chan, A.; Matthaes, P.; Wagener, C.: Biliary glyco protein, a potential human cell adhesion molecule, is down-regulated in colorectal carcinomas. Proc. Nat. Acad. Sci. 90:10744-10748, 1993.
Poy, M. N.; Yang, Y.; Rezaei, K.; Fernstrom, M. A.; Lee, A. D.; Kido, Y.; Erickson, S. K.; Najjar, S. M.: CEACAM1 regulates insulin clearance in liver. Nature Genet. 30: 270-276, 2002
Robbins, J.; Robbins, P. F.; Kozak, C. A.; Callahan, R.: Themouse biliary glyco protein gene (Bgp): partial nucleotide sequence, expression, and chromosomal assignment. Genomics 10: 583-587, 1991.
Thompson, J. A.; Grunert, F.; Zimmermann, W.: Carcino embryonic antigen gene family: molecular biology and clinical perspectives. J. Clin. Lab. Anal. 5: 344-366, 1991.
Bierhuizen, M. F. A.; Mattei, M.-G.; Fukuda, M.: Expression of the developmental I antigen by a cloned human cDNA encoding a member of a beta-1,6-N-acetylglucosaminyltransferase gene family. Genes-Dev. 7: 468-478, 1993.
Lin-Chu, M.; Broadberry, R. E.; Okubo, Y.; Tanaka, M.: The i phenotype and congenital cataracts among Chinese in Taiwan (Letter) Transfusion 31:676-677, 1991.
Ogata, H.; Okubo, Y.; Akabane, T.: Phenotype i associated with congenital cataract in Japanese. Transfusion 19: 166-168, 1979.
Yeh, J.-C.; Ong, E.; Fukuda, M.: Molecular cloning and expression of a novel beta-1,6-N-acetylglucosaminyltransferase that forms core 2, core 4, and I branches. J. Biol. Chem. 274: 3215-3221, 1999
Yu, L.-C.; Twu, Y.-C.; Chang, C.-Y.; Lin, M.: Molecular basis of the adult i phenotype and the gene responsible for the expression of the human blood group I antigen. Blood 98: 3840-3845, 2001.
Azuma, N.; Nishina, S.; Yanagisawa, H.; Okuyama, T.; Yamada, M.: PAX6 missense mutation in isolated foveal hypoplasia. (Letter) Nature Genet. 13: 141-142, 1996.
Curran, R. E.; Robb, R. M.: Isolated foveal hypoplasia. Arch. Ophthal. 94: 48-50, 1976.
O'Donnell, F. E., Jr.; Pappas, H. R.: Autosomal dominant foveal hypoplasia and presenile cataracts: a new syndrome. Arch. Ophthal. 100:279-281, 1982.
Tommerup, N.; Schempp, W.; Meinecke, P.; Pedersen, S.; Bolund, L.; Brandt, C.; Goodpasture, C.; Guldberg, P.; Held, K.; Reinwein, H.; Saugstad, O. D.; Scherer, G.; Skjeldal, O.; Toder, R.; Westvik, J.; van der Hagen, C. B.; Wolf, U.: Assignment of an autosomal sex reversal locus (SRA1) and campomelic dysplasia (CMPD1) to 17q24.3-q25.1. Nature Genet. 4: 170-174, 1993.
Angle, C. R.: Congenital bowing and angulation of the long bones. Pediatrics 13:257-268, 1954.
Bain, A. D.; Barrett, H. S.: Congenital bowing of the long bones: report of a case. Arch. Dis. Child. 34: 516-524, 1959.
Bell, D. M.; Leung, K. K. H.; Wheatley, S. C.; Ng, L. J.; Zhou, S.; Ling, K. W.; Sham, M. H.; Koopman, P.; Tam, P. P. L.; Cheah, K. S. E.: SOX9 directly regulates the type-II collagen gene. Nature Genet. 16: 174-178, 1997.
Bi, W.; Huang, W.; Whitworth, D. J.; Deng, J. M.; Zhang, Z.; Behringer, R. R.; de Crombrugghe, B.: Haploinsufficiency of Sox9 results in defective cartilage primordia and premature skeletal mineralization. Proc. Nat. Acad. Sci. 98: 6698-6703, 2001.
Bishop, C. E.; Whitworth, D. J.; Qin, Y.; Agoulnik, A. I.; Agoulnik, I. U.; Harrison, W. R.; Behringer, R. R.; Overbeek, P. A.: A transgenic insertion upstream of Sox9 is associated with dominant XX sex reversal in the mouse. Nature Genet. 26: 490-494, 2000.
Caffey, J. P.: Prenatal bowing and thickening of tubular bones, with multiple cutaneous dimples in arms and legs: a congenital syndrome of mechanical origin. Am. J. Dis. Child. 74: 543-562, 1947.
Cameron, F. J.; Hageman, R. M.; Cooke-Yarborough, C.; Kwok, C.; Goodwin, L. L.; Sillence, D. O.; Sinclair, A. H.: A novel germ line mutation in SOX9 causes familial campomelic dysplasia and sex reversal. Hum. Molec. Genet. 5: 1625-1630, 1996.
Cameron, F. J.; Sinclair, A. H.: Mutations in SRY and SOX9: testis-determining genes. Hum. Mutat. 9: 388-395, 1997.
Cooke, C. T.; Mulcahy, M. T.; Cullity, G. J.; Watson, M.; Sprague, P.: Campomelic dysplasia with sex reversal: morphological and cytogenetic studies of a case. Pathology 17: 526-529, 1985.
Cremin, B. J.; Orsmond, G.; Beighton, P.: Autosomal recessive inheritance in camptomelic dwarfism. (Letter) Lancet I: 488-489, 1973.
Dagna Bricarelli, F.; Fraccaro, M.; Lindsten, J.; Muller, U.; Baggio, P.; Carbone, L. D. L.; Hjerpe, A.; Lindgren, F.; Mayerova, A.; Ringertz, H.; Ritzen, E. M.; Rovetta, D. C.; Sicchero, C.; Wolf, U.: Sex-reversed XY females with campomelic dysplasia are H—Y negative. Hum. Genet. 57: 15-22, 1981.
Ebensperger, C.; Jager, R. J.; Lattermann, U.; Dagna Bricarelli, F.; Keutel, J.; Lindsten, J.; Rehder, H.; Muller, U.; Wolf, U.: No evidence of mutations in four candidate genes for male sex determination/differentiation in sex-reversed XY females with campomelic dysplasia. Ann. Genet. 34:233-238, 1991.
Fontaine, G.; Walbaum, R.; Farriaux, J. P.; Tilmont, P.; Peuzin, F.; Delecour, M.: Le conseil genetique dans la dysplasie campomelique (a propos de deux observations). J. Genet. Hum. 28: 267-279, 1980.
Foster, J. W.; Dominguez-Steglich, M. A.; Guioli, S.; Kwok, C.; Weller, P. A.; Stevanovic, M.; Weissenbach, J.; Mansour, S.; Young, I. D.; Goodfellow, P. N.; Brook, J. D.; Schafer, A. J.: Campomelic dysplasia and autosomal sex reversal caused by mutations in an SRY-related gene. Nature 372: 525-530, 1994.
Friedrich, U.; Schaefer, E.; Meinecke, P.: Campomelic dysplasia without overt campomelia. Clin. Dysmorph. 1: 172-178, 1992.
Gasca, S.; Canizares, J.; de Santa Barbara, P.; Mejean, C.; Poulat, F.; Berta, P.; Boizet-Bonhoure, B.: A nuclear export signal within the high mobility group domain regulates the nucleocytoplasmic translocation of SOX9 during sexual determination. Proc. Nat. Acad. Sci. 99: 11199-11204, 2002.
Glass, R. B. J.; Rosenbaum, K. N.: Acampomelic campomelic dysplasia: further radiographic variations. Am. J. Med. Genet. 69: 29-32, 1997.
Hall, B.; Spranger, J. W.: Campomelic dysplasia: further elucidation of a distinct entity. Am. J. Dis. Child. 134: 285-289, 1980
Hoefnagel, D.; Wurster-Hill, D. H.; Dupree, W. B.; Benirschke, K.; Fuld, G. L.: Camptomelic dwarfism associated with XY-gonadal dysgenesis and chromosome anomalies. Clin. Genet. 13: 489-499, 1978.
Houston, C. S.; Opitz, J. M.; Spranger, J. W.; Macpherson, R. I.; Reed, M. H.; Gilbert, E. F.; Herrmann, J.; Schinzel, A.: The campomelic syndrome: review, report of 17 cases, and follow-up on the currently 17-year-old boy first reported by Maroteaux et al in 1971. Am. J. Med. Genet. 15: 3-28, 1983.
Hovmoller, M. L.; Osuna, A.; Eklof, O.; Fredga, K.; Hjerpe, A.; Lindsten, J.; Ritzen, M.; Stanescu, V.; Svenningsen, N.: Camptomelic dwarfism. A genetically determined mesenchymal disorder combined with sex reversal. Hereditas 86: 51-62, 1977.
Huang, W.; Chung, U.; Kronenberg, H. M.; de Crombrugghe, B.: The chondrogenic transcription factor Sox9 is a target of signaling by the parathyroid hormone-related peptide in the growth plate of endochondral bones. Proc. Nat. Acad. Sci. 98: 160-165, 2001.
Kanai, Y.; Koopman, P.: Structural and functional characterization of the mouse Sox9 promoter: implications for campomelic dysplasia. Hum. Molec. Genet. 8: 691-696, 1999.
Medema, R. H.; Kops, G. J. P. L.; Bos, J. L.; Burgering, B. M. T.: AFX-like forkhead transcription factors mediate cell-cycle regulation by Ras and PKB through p27(kipl). Nature 404: 782-787, 2000.
Bourgeois, C.; Robert, B.; Rebourcet, R.; Mondon, F.; Mignot, T.-M.; Duc-Goiran, P.; Ferre, F.: Endothelin-1 and ET(A) receptor expression in vascular smooth muscle cells from human placenta: a new ET(A) receptor messenger ribonucleic acid is generated by alternative splicing of exon 3. J. Clin. Endocr. Metab. 82: 3116-3123, 1997.
Amato, F.; Warnes, G. M.; Kirby, C. A.; Norman, R. J.: Infertility caused by hCG autoantibody. J. Clin. Endocr. Metab. 87: 993-997, 2002.
Naylor, S. L.; Chin, W. W.; Goodman, H. M.; Lalley, P. A.; Grzeschik, K.-H.; Sakaguchi, A. Y.: Chromosome assignment of the genes encoding the alpha and beta subunits of the glyco protein hormones in man and mouse. Somat. Cell Genet. 9: 757-770, 1983.
Mammarella, S.; Romano, F.; Di Valerio, A.; Creati, B.; Esposito, D. L.; Palmirotta, R.; Capani, F.; Vitullo, P.; Volpe, G.; Battista, P.; Della Loggia, F.; Mariani-Costantini, R.; Cama, A.: Interaction between the G1057D variant of IRS-2 and overweight in the pathogenesis of type 2 diabetes. Hum. Molec. Genet. 9: 2517-2521, 2000.
Barker, P. E.; Shipp, M. A.; D'Adamio, L.; Masteller, E. L.; Reinherz, E. L.: The common acute lymphoblastic leukemia antigen gene maps to chromosomal region 3(q21-q27). J. Immun. 142: 283-287, 1989.
D'Adamio, L.; Shipp, M. A.; Masteller, E. L.; Reinherz, E. L.: Organization of the gene encoding common acute lymphoblastic leukemia antigen (neutral endopeptidase 24.11): multiple mini exons and separate 5-prime untranslated regions. Proc. Nat. Acad. Sci. 86: 7103-7107, 1989.
Debiec, H.; Guigonis, V.; Mougenot, B.; Decobert, F.; Haymann, J.-P.; Bensman, A.; Deschenes, G.; Ronco, P. M.: Antenatal membranous glomerulonephritis due to antineutral endopeptidase antibodies. New Eng. J. Med. 346: 2053-2060, 2002.
Letarte, M.; Vera, S.; Tran, R.; Addis, J. B. L.; Onizuka, R. J.; Quackenbush, E. J.; Jongeneel, C. V.; McInnes, R. R.: Common acute lymphocytic leukemia antigen is identical to neutral endopeptidase. J. Exp. Med. 168: 1247-1253, 1988.
Shipp, M. A.; Vijayaraghavan, J.; Schmidt, E. V.; Masteller, E. L.; D'Adamio, L.; Hersh, L. B.; Reinherz, E. L.: Common acute lymphoblastic leukemia antigen (CALLA) is active neutral endopeptidase 24.11 (‘enkephalinase’): direct evidence by cDNA transfection analysis. Proc. Nat. Acad. Sci. 86:297-301, 1989.
Tran-Paterson, R.; Willard, H. F.; Letarte, M.: The common acute lymphoblastic leukemia antigen (neutral endopeptidase—3.4.24.11) gene is located on human chromosome 3. Cancer Genet. Cytogenet. 42:129-134, 1989.
Lahn, B. T.; Page, D. C.: Four evolutionary strata on the human X chromosome. Science 286: 964-967, 1999.
Lee, F. A.; Issacs, H.; Strauss, J.: The ‘camptomelic’ syndrome. Short life-span dwarfism with respiratory distress, hypotonia, peculiarfacies, and multiple skeletal and cartilaginous deformities. Am. J. Dis. Child. 124: 485-496, 1972.
Lynch, S. A.; Gaunt, M. L.; Minford, A. M. B.: Campomelic dysplasia: evidence of autosomal dominant inheritance. J. Med. Genet. 30: 683-686, 1993.
Macpherson, R. I.; Skinner, S. A.; Donnenfeld, A. E.: A campomelic campomelic dysplasia. Pediat. Radiol. 20: 90-93, 1989.
Mansour, S.; Hall, C. M.; Pembrey, M. E.; Young, I. D.: A clinical and genetic study of campomelic dysplasia. J. Med. Genet. 32: 415-420, 1995.
Maraia, R.; Saal, H. M.; Wangsa, D.: A chromosome 17q de novo paracentric inversion in a patient with campomelic dysplasia; case report and etiologic hypothesis. Clin. Genet. 39: 401-408, 1991.
Maroteaux, P.; Spranger, J. W.; Opitz, J. M.; Kucera, J.; Lowry, R. B.; Schimke, R. N.; Kagan, S. M.: Le syndrome campomelique. Presse Med. 22: 1157-1162, 1971.
Meyer, J.; Sudbeck, P.; Held, M.; Wagner, T.; Schmitz, M. L.; Bricarelli, F. D.; Eggermont, E.; Friedrich, U.; Haas, O. A.; Kobelt, A.; Leroy, J. G.; van Maldergem, L.; Michel, E.; Mitulla, B.; Pfeiffer, R. A.; Schinzel, A.; Schmidt, H.; Scherer, G.: Mutational analysis of the SOX9 gene in campomelic dysplasia and autosomal sex reversal: lack of genotype/phenotype correlations. Hum. Molec. Genet. 6: 91-98, 1997.
Moedjono, S. J.; Crandall, B. F.; Sparkes, R. S.; Feldman, G. M.; Austin, G. E.; Perry, S.: The campomelic syndrome in a singleton and monozygotic twins. Clin. Genet. 18: 397-401, 1980.
Moog, U.; Jansen, N. J. G.; Scherer, G.; Schrander-Stumpel, C. T. R. M.: Acampomelic campomelic syndrome. Am. J. Med. Genet. 104:239-245, 2001
Morais da Silva, S.; Hacker, A.; Harley, V.; Goodfellow, P.; Swain, A.; Lovell-Badge, R.: Sox9 expression during gonadal development implies a conserved role for the gene in testis differentiation in mammals and birds. Nature Genet. 14: 62-68, 1996.
Murakami, S.; Kan, M.; McKeehan, W. L.; de Crombrugghe, B.: Up-regulation of the chondrogenic Sox9 gene by fibroblast growth factors is mediated by the mitogen-activated protein kinase pathway. Proc. Nat. Acad. Sci. 97: 1113-1118, 2000.
Ninomiya, S.; Isomura, M.; Narahara, K.; Seino, Y.; Nakamura, Y.: Isolation of a testis-specific cDNA on chromosome 17q from a region adjacent to the breakpoint of t(12;17) observed in a patient with acampomelic campomelic dysplasia and sex reversal. Hum. Molec. Genet. 5: 69-72, 1996.
Ninomiya, S.; Yokoyama, Y.; Teraoka, M.; Mori, R.; Inoue, C.; Yamashita, S.; Tamai, H.; Funato, M.; Seino, Y.: A novel mutation (296 del G) of the SOX9 gene in a patient with campomelic syndrome and sex reversal. Clin. Genet. 58: 224-227, 2000.
Olney, P. N.; Kean, L. S.; Graham, D.; Elsas, L. J.; May, K. M.: Campomelic syndrome and deletion of SOX9. Am. J. Med. Genet. 84:20-24, 1999.
Ozkilic, A.; Seven, M.; Yuksel, A.: A case of acampomelic campomelic dysplasia. Genet. Counsel. 13: 23-28, 2002.
Patel, M.; Dorman, K. S.; Zhang, Y.-H.; Huang, B.-L.; Arnold, A. P.; Sinsheimer, J. S.; Vilain, E.; McCabe, E. R. B.: Primate DAX1, SRY, and SOX9: evolutionary stratification of sex-determination pathway. Am. J. Hum. Genet. 68: 275-280, 2001.
Pfeifer, D.; Kist, R.; Dewar, K.; Devon, K.; Lander, E. S.; Birren, B.; Korniszewski, L.; Back, E.; Scherer, G.: Campomelic dysplasia translocation breakpoints are scattered over 1 Mb proximal to SOX9: evidence for an extended control region. Am. J. Hum. Genet. 65:111-124, 1999.
Puck, S. M.; Haseltine, F. P.; Francke, U.: Absence of H—Y antigen in an XY female with campomelic dysplasia. Hum. Genet. 57: 23-27, 1981.
Rimoin, D. L.: Personal Communication. Torrance, Calif. Aug. 12, 1976.
Rodriguez, J. I.: Vascular anomalies in campomelic syndrome. Am. J. Med. Genet. 46: 185-192, 1993.
Savarirayan, R.; Bankier, A.: Acampomelic campomelic dysplasia with de novo 5q; 17q reciprocal translocation and severe phenotype. J. Med. Genet. 35: 597-599, 1998
Schimke, R. N.: XY sex-reversed campomelia—possibly an X-linked disorder? (Letter) Clin. Genet. 16: 62-63, 1979.
Shafai, T.; Schwartz, L.: Camptomelic syndrome in siblings. J. Pediat. 89: 512-513, 1976.
Spranger, J.: Advances in bone dysplasias. (Abstract) Sixth Int. Cong. Hum. Genet. Jerusalem, 1981.
Stuve, A.; Wiedemann, H.-R.: Congenital bowing of the long bones in two sisters. (Letter) Lancet I: 495, 1971.
Sudbeck, P.; Schmitz, M. L.; Baeuerle, P. A.; Scherer, G.: Sex reversal by loss of the C-terminal transactivation domain of human SOX9. Nature Genet. 13: 230-232, 1996.
Thong, M.-K.; Scherer, G.; Kozlowski, K.; Haan, E.; Morris, L.: Acampomelic campomelic dysplasia with SOX9 mutation. Am. J. Med. Genet. 93: 421-425, 2000.
Thurmon, T. F.; De Fraites, E. B.; Anderson, E. E.: Familial-campomelic dwarfism. J. Pediat. 83: 841-843, 1973.
Vidal, V. P. I.; Chaboissier, M.-C.; de Rooij, D. G.; Schedl, A.: Sox9 induces testis development in XX transgenic mice. Nature Genet. 28: 216-217, 2001.
Wagner, T.; Wirth, J.; Meyer, J.; Zabel, B.; Held, M.; Zimmer, J.; Pasantes, J.; Dagna Bricarelli, F.; Keutel, J.; Hustert, E.; Wolf, U.; Tommerup, N.; Schempp, W.; Scherer, G.: Autosomal sex reversal and campomelic dysplasia are caused by mutations in and around the SRY-related gene SOX9. Cell 79: 1111-1120, 1994.
Weller, S. D. V.: Hypophosphatasia with congenital dimples. Proc. Roy. Soc. Med. 52: 637, 1959.
Wirth, J.; Wagner, T.; Meyer, J.; Pfeiffer, R. A.; Tietze, H.-U.; Schempp, W.; Scherer, G.: Translocation breakpoints in three patients with campomelic dysplasia and autosomal sex reversal map more than 130 kb from SOX9. Hum. Genet. 97: 186-193, 1996.
Wright, E.; Hargrave, M. R.; Christiansen, J.; Cooper, L.; Kun, J.; Evans, T.; Gangadharan, U.; Greenfield, A.; Koopman, P.: The Sry-related gene Sox9 is expressed during chondrogenesis in mouse embryos. Nature Genet. 9: 15-20, 1995.
Gillessen-Kaesbach, G.; Demuth, S.; Thiele, H.; Theile, U.; Lich, C.; Horsthemke, B.: A previously unrecognised phenotype characterised by obesity, muscular hypotonia, and ability to speak in patients with Angelman syndrome caused by an imprinting defect. Europ. J. Hum. Genet. 7: 638-644, 1999.
Acosta, J.; Hettinga, J.; Fluckiger, R.; Krumrei, N.; Goldfine, A.; Angarita, L.; Halperin, J.: Molecular basis for a link between complement and the vascular complications of diabetes. Proc. Nat. Acad. Sci. 97: 5450-5455, 2000.
Bickmore, W. A.; Longbottom, D.; Oghene, K.; Fletcher, J. M.; van Heyningen, V.: Colocalization of the human CD59 gene to 11p13 with the MIC11 cell surface antigen. Genomics 17: 129-135, 1993.
Davies, A.; Simmons, D. L.; Hale, G.; Harrison, R. A.; Tighe, H.; Lachmann, P. J.; Waldmann, H.: CD59, an LY-6-like protein expressed in human lymphoid cells, regulates the action of the complement membrane attack complex on homologous cells. J. Exp. Med. 170: 637-654, 1989.
Forsberg, U. H.; Bazil, V.; Stefanova, I.; Schroder, J.: Gene-for human CD59 (likely Ly-6 homologue) is located on the short arm of chromosome 11. Immunogenetics 30: 188-193, 1989.
Harada, R.; Okada, N.; Fujita, T.; Okada, H.: Purification of 1F5 antigen that prevents complement attack on homologous cell membranes. J. Immun. 144: 1823-1828, 1990.
Heckl-Ostreicher, B.; Ragg, S.; Drechsler, M.; Scherthan, H.; Royer-Pokora, B.: Localization of the human CD59 gene by fluorescence in situ hybridization and pulsed-field gel electrophoresis. Cytogenet. Cell Genet. 63:144-146, 1993
Holt, D. S.; Botto, M.; Bygrave, A. E.; Hanna, S. M.; Walport, M. J.; Morgan, B. P.: Targeted deletion of the CD59 gene causes spontaneous intravascular hemolysis and hemoglobinuria. Blood 98: 442-449, 2001.
Holt, D. S.; Powell, M. B.; Rushmere, N. K.; Morgan, B. P.: Genomic structure and chromosome location of the gene encoding mouse CD59. Cytogenet. Cell Genet. 89: 264-267, 2000.
Huppi, K.; Duncan, R.; Potter, M.: Myc-1 is centromeric to the linkage group Ly-6-Sis-Gdc-1 on mouse chromosome 15. Immunogenetics 27:215-219, 1988.
Kamiura, S.; Nolan, C. M.; Meruelo, D.: Long-range physical map of the Ly-6 complex: mapping the Ly-6 multi gene family by field-inversion and two-dimensional gel electrophoresis. Genomics 12: 89-105, 1992.
Low, M. G.; Saltiel, A. R.: Structural and functional roles of glycosyl-phosphatidylinositol in membranes. Science 239: 268-275, 1988.
Mahoney, J. F.; Urakaze, M.; Hall, S.; DeGasperi, R.; Chang, H.-M.; Sugiyama, E.; Warren, C. D.; Borowitz, M.; Nicholson-Weller, A.; Rosse, W. F.; Yeh, E. T. H.: Defective glycosylphosphatidylinositol anchor synthesis in paroxysmal nocturnal hemoglobinuria granulocytes. Blood 79:1400-1403, 1992.
Mao, M.; Yu, M.; Tong, J.-H.; Ye, J.; Zhu, J.; Huang, Q.-H.; Fu, G.; Yu, L.; Zhao, S.-Y.; Waxman, S.; Lanotte, M.; Wang, Z.-Y.; Tan, J.-Z.; Chan, S.-J.; Chen, Z.: RIG-E, a human homolog of the murine Ly-6 family, is induced by retinoic acid during the differentiation of acute promyelocytic leukemia cell. Proc. Nat. Acad. Sci. 93:5910-5914, 1996.
Meri, S.; Morgan, B. P.; Davies, A.; Daniels, R. H.; Olavesen, M. G.; Waldmann, H.; Lachmann, P. J.: Human protectin (CD59), an 18,000-20,000 MW complement lysis restricting factor, inhibits C5b-8 catalysed insertion of C9 into lipid bilayers. Immunology 71: 1-9, 1990.
Meri, S.; Morgan, B. P.; Wing, M.; Jones, J.; Davies, A.; Podack, E.; Lachmann, P. J.: Human protectin (CD59), an 18-20-kD homologous complement restriction factor, does not restrict perforin-mediated lysis. J. Exp. Med. 172: 367-370, 1990.
Motoyama, N.; Okada, N.; Yamashina, M.; Okada, H.: Paroxysmal nocturnal hemoglobinuria due to hereditary nucleotide deletion in the HRF20 (CD59) gene. Europ. J. Immun. 22: 2669-2673, 1992.
Okada, N.; Harada, R.; Fujiita, T.; Okada, H.: A novel membrane glyco protein capable of inhibiting membrane attack by homologous complement. Int. Immun. 1: 205-208, 1989.
Szpirer, C.; Riviere, M.; Cortese, R.; Nakamura, T.; Islam, M. Q.; Levan, G.; Szpirer, J.: Chromosomal localization in man and rat of the genes encoding the liver-enriched transcription factors C/EBP, DBP, and HNF1/LFB-1 (CEBP, DBP, and transcription factor 1, TCF1, respectively) and of the hepatocyte growth factor/scatter factor gene (HGF). Genomics 13: 293-300, 1992.
Boorstein, W. R.; Vamvakopoulos, N. C.; Fiddes, J. C.: Human chorionic gonadotropin beta-subunit is encoded by at least eight genes arranged in tandem and inverted pairs. Nature 300: 419-422, 1982.
Fiddes, J. C.; Goodman, H. M.: The cDNA for the beta-subunit of human chorionic gonadotropin suggests evolution of a gene by read through into the 3-prime-untranslated region. Nature 286: 684-687, 1980.
Graham, M. Y.; Otani, T.; Boime, I.; Olson, M. V.; Carle, G. F.; Chaplin, D. D.: Cosmid mapping of the human chorionic gonadotropinbeta subunit genes by field-inversion gel electrophoresis. Nucleic Acids Res. 15: 4437-4448, 1987.
Julier, C.; Weil, D.; Couillin, P.; Cote, J. C.; Boue, A.; Thririon, J. P.; Kaplan, J. C.; Junien, C.: Confirmation of the assignment of the genes coding for human chorionic gonadotropin beta subunit to chromosome 19. (Abstract) Cytogenet. Cell Genet. 37: 501-502, 1984.
Julier, C.; Weil, D.; Couillin, P.; Cote, J. C.; Van Cong, N.; Foubert, C.; Boue, A.; Thirion, J. P.; Kaplan, J. C.; Junien, C.: The beta chorionic gonadotropin-beta luteinizing gene cluster maps to human chromosome 19. Hum. Genet. 67: 174-177, 1984.
Lunardi-Iskander, Y.; Bryant, J. L.; Zeman, R. A.; Lam, V. H.; Samaniego, F.; Besnier, J. M.; Hermans, P.; Thierry, A. R.; Gill, P.; Gallo, R. C.: Tumorigenesis and metastasis of neoplastic Kaposi'ssarcoma cell line in immunodeficient mice blocked by a human pregnancy hormone. Nature 375: 64-68, 1995.
Policastro, P.; Ovitt, C. E.; Hoshina, M.; Fukuoka, H.; Boothby, M. R.; Biome, I.: The beta-subunit of human chorionic gonadotropin is encoded by multiple genes. J. Biol. Chem. 258: 11492-11499, 1983.
Policastro, P. F.; Daniels-McQueen, S.; Carle, G.; Boime, I.: A map of the hCG-beta-LH-beta gene cluster. J. Biol. Chem. 261:5907-5916, 1986.
Talmadge, K.; Vamvakopoulos, N. C.; Fiddes, J. C.: Evolution of the genes for the beta subunits of human chorionic gonadotropin and luteinizing hormone. Nature 307: 37-40, 1984.
Warburton, D.; Gersen, S.; Yu, M.-T.; Jackson, C.; Handelin, B.; Housman, D.: Monochromosomal rodent-human hybrids from microcell fusion of human lymphoblastoid cells containing an inserted dominant selectable marker. Genomics 6: 358-366, 1990.
Seri, M.; Celli, I.; Betsos, N.; Claudiani, F.; Camera, G.; Romeo, G.: A cys634gly substitution of the RET protooncogene in a family with recurrence of multiple endocrine neoplasia type 2A and cutaneous lichen amyloidosis. Clin. Genet. 51: 86-90, 1997.
Hofstra, R. M. W.; Sijmons, R. H.; Stelwagen, T.; Stulp, R. P.; Kousseff, B. G.; Lips, C. J. M.; Steijlen, P. M.; Van Voorst Vader, P. C.; Buys, C. H. C. M.: RET mutation screening in familial cutaneous lichen amyloidosis and in skin amyloidosis associated with multiple endocrine neoplasia. J. Invest. Derm. 107: 215-218, 1996.
Fantes, J. A.; Bickmore, W. A.; Fletcher, J. M.; Ballesta, F.; Hanson, I. M.; van Heyningen, V.: Submicroscopic deletions at the WAGR locus, revealed by nonradioactive in situ hybridization. Am. J. Hum. Genet. 51: 1286-1294, 1992
Glaser, T.; Jepeal, L.; Edwards, J. G.; Young, S. R.; Favor, J.; Maas, R. L.: PAX6 gene dosage effect in a family with congenital cataracts, aniridia, anophthalmia and central nervous system defects. Nature Genet. 7: 463-471, 1994.
Glaser, T.; Lane, J.; Housman, D.: A mouse model of the aniridia-Wilms tumor deletion syndrome. Science 250: 823-827, 1990.
Gronskov, K.; Olsen, J. H.; Sand, A.; Pedersen, W.; Carlsen, N.; Jylling, A. M. B.; Lyngbye, T.; Brondum-Nielsen, K.; Rosenberg, T.: Population-based risk estimates of Wilms tumor in sporadic aniridia: a comprehensive mutation screening procedure of PAX6 identifies 80% of mutations in aniridia. Hum. Genet. 109: 11-18, 2001.
Hanson, I. M.; Seawright, A.; Hardman, K.; Hodgson, S.; Zaletayev, D.; Fekete, G.; van Heyningen, V.: PAX6 mutations in aniridia. Hum. Molec. Genet. 2: 915-920, 1993.
Hill, R. E.; Favor, J.; Hogan, B. L. M.; Ton, C. C. T.; Saunders, G. F.; Hanson, I. M.; Prosser, J.; Jordan, T.; Hastie, N. D.; van Heyningen, V.: Mouse small eye results from mutations in a paired-like homeobox-containing gene. Nature 354: 522-525, 1991.
Jordan, T.; Hanson, I.; Zaletayev, D.; Hodgson, S.; Prosser, J.; Seawright, A.; Hastie, N.; van Heyningen, V.: The human PAX6 gene is mutated in two patients with aniridia. Nature Genet. 1: 328-332, 1992.
Karpen, G. H.: Position effect variegation and the new biology of heterochromatin. Curr. Opin. Genet. Dev. 4: 281-291, 1994.
Lyon, M. F.: Personal Communication. Harwell, England Jun. 9, 1988.
Martha, A.; Strong, L. C.; Ferrell, R. E.; Saunders, G. F.: Three novel aniridia mutations in the human PAX6 gene. Hum. Mutat. 6:44-49, 1995.
Matsuo, T.; Osumi-Yamashita, N.; Noji, S.; Ohuchi, H.; Koyama, E.; Myokai, F.; Matsuo, N.; Taniguchi, S.; Doi, H.; Iseki, S.; Ninomiya, Y.; Fujiwara, M.; Watanabe, T.; Eto, K.: A mutation in the Pax-6 gene in rat small eye is associated with impaired migration of midbrain crest cells. Nature Genet. 3: 299-304, 1993.
Oliver, M. D.; Dotan, S. A.; Chemke, J.; Abraham, F. A.: Isolated foveal hypoplasia. Brit. J. Ophthal. 71: 926-930, 1987.
Prosser, J.; van Heyningen, V.: PAX6 mutations reviewed. Hum. Mutat. 11: 93-108, 1998.
Quiring, R.; Walldorf, U.; Kloter, U.; Gehring, W. J.: Homology of the eyeless gene of Drosophila to the small eye gene in mice and aniridia in humans. Science 265: 785-789, 1994.
Salvini-Plawen, L.; Mayr, E.: On the evolution of photoreceptors and eyes. In: Hecht, M. K.; Steere, W.; Wallace, B.: Evolutionary Biology. New York: Plenum Pub. (pub.) 10: 1977. Pp. 207-263.
Schedl, A.; Ross, A.; Lee, M.; Engelkamp, D.; Rashbass, P.; van Heyningen, V.; Hastie, N. D.: Influence of PAX6 gene dosage on development: overexpression causes severe eye abnormalities. Cell 86: 71-82, 1996.
Stone, D. L.; Kenyon, K. R.; Green, W. R.; Ryan, S. J.: Congenital central corneal leukoma (Peters' anomaly). Am. J. Ophthal. 81: 173-193, 1976.
van der Meer-de Jong, R.; Dickinson, M. E.; Woychik, R. P.; Stubbs, L.; Hetherington, C.; Hogan, B. L. M.: Location of the gene involving the small eye mutation on mouse chromosome 2 suggests homology with human aniridia 2 (AN2). Genomics 7: 270-275, 1990.
Cowan, C. A.; Yokoyama, N.; Bianchi, L. M.; Henkemeyer, M.; Fritzsch, B.: EphB2 guides axons at the midline and is necessary for normal vestibular function. Neuron 26: 417-430, 2000.
Ton, C. C. T.; Hirvonen, H.; Miwa, H.; Weil, M. M.; Monaghan, P.; Jordan, T.; van Heyningen, V.; Hastie, N. D.; Meijers-Heijboer, H.; Drechsler, M.; Royer-Pokora, B.; Collins, F.; Swaroop, A.; Strong, L. C.; Saunders, G. F.: Positional cloning and characterization of a paired box- and homeobox-containing gene from the aniridia region. Cell 67:1059-1074, 1991.
Schmickel, R. D.: Contiguous gene syndromes: a component of recognizable syndromes. J. Pediat. 109: 231-241, 1986.
Valent, A.; Danglot, G.; Bernheim, A.: Mapping of the tyrosine kinase receptors trkA (NTRK1), trkB (NTRK2) and trkC (NTRK3) to human chromosomes 1q22, 9q22 and 15q25 by fluorescence in situ hybridization. Europ. J. Hum. Genet. 5: 102-104, 1997.
Day, I. N. M.; Hinks, L. J.; Thompson, R. J.: The structure of the gene encoding protein gene product 9.5 (PGP9.5), a neuron-specific ubiquitin C-terminal hydrolase. Biochem. J. 268: 521-524, 1990.
Day, I. N. M.; Thompson, R. J.: Molecular cloning of cDNA coding for human PGP 9.5 protein: a novel cytoplasmic marker for neurones and neuroendocrine cells. FEBS Lett. 210: 157-160, 1987.
Doran, J. F.; Jackson, P.; Kynoch, P.; Thompson, R. J.: Isolation of PGP 9.5, a new human neurone-specific protein detected by high-resolution two-dimensional electrophoresis. J. Neurochem. 40: 1542-1547, 1983.
Edwards, Y. H.; Fox, M. F.; Povey, S.; Hinks, L. J.; Day, I. N. M.; Thompson, R. J.: The gene for human neuron specific ubiquitin C-terminal hydrolase maps to chromosome 4p14. (Abstract) Cytogenet. Cell Genet. 58: 1886-1887, 1991.
Kurihara, L. J.; Kikuchi, T.; Wada, K.; Tilghman, S. M.: Loss of Uch-L1 and Uch-L3 leads to neurodegeneration, posterior paralysis and dysphagia. Hum. Molec. Genet. 10: 1963-1970, 2001.
Kurihara, L. J.; Semenova, E.; Levorse, J. M.; Tilghman, S. M.: Expression and functional analysis of Uch-L3 during mouse development. Molec. Cell. Biol. 20: 2498-2504, 2000.
Leroy, E.; Boyer, R.; Auburger, G.; Leube, B.; Ulm, G.; Mezey, E.; Harta, G.; Brownstein, M. J.; Jonnalagada, S.; Chernova, T.; Dehejia, A.; Lavedan, C.; Gasser, T.; Steinbach, P. J.; Wilkinson, K. D.; Polymeropoulos, M. H.: The ubiquitin pathway in Parkinson's disease. (Letter) Nature 395:451-452, 1998.
Lincoln, S.; Vaughan, J.; Wood, N.; Baker, M.; Adamson, J.; Gwinn-Hardy, K.; Lynch, T.; Hardy, J.; Farrer, M.: Low frequency of pathogenic mutations in the ubiquitin carboxyterminal hydrolase gene in familial Parkinson's disease. Neuroreport 10: 427-429, 1999.
MacDonald, M. E.: Gadzooks! Nature Genet. 23: 10-11, 1999.
Saigoh, K.; Wang, Y.-L.; Suh, J.-G.; Yamanishi, T.; Sakai, Y.; Kiyosawa, H.; Harada, T.; Ichihara, N.; Wakana, S.; Kikuchi, T.; Wada, K.: Intragenic deletion in the gene encoding ubiquitin carboxy-terminal hydrolase in gad mice. Nature Genet. 23: 47-51, 1999.
Suh, J. G.; Yamanishi, T.; Matsui, K.; Tanaka, K.; Wada, K.: Mapping of the gracile axonal dystrophy (gad) gene to a region between D5Mit197 and D5Mit113 on proximal mouse chromosome 5. Genomics 27:549-551, 1995.
Wilkinson, K. D.; Lee, K. M.; Deshpande, S.; Duerksen-Hughes, P.; Boss, J. M.; Pohl, J.: The neuron-specific protein PGP 9.5 is a ubiquitin carboxyl-terminal hydrolase. Science 246: 670-672, 1989.
Yamazaki, K.; Wakasugi, N.; Tomita, T.; Kikuchi, T.; Mukoyama, M.; Ando, K.: Gracile axonal dystrophy (GAD), a new neurological mutant in the mouse. Proc. Soc. Exp. Biol. Med. 187: 209-215, 1988
Morii, K.; Tanaka, R.; Takahashi, Y.; Minoshima, S.; Fukuyama, R.; Shimizu, N.; Kuwano, R.: Structure and chromosome assignment of human S100 alpha and beta subunit genes. Biochem. Biophys. Res. Commun. 175: 185-191, 1991.
Ip, N. Y.; Stitt, T. N.; Tapley, P.; Klein, R.; Glass, D. J.; Fandl, J.; Greene, L. A.; Barbacid, M.; Yancopoulos, G. D.: Similarities and differences in the way neurotrophins interact with the Trk receptors in neuronal and nonneuronal cells. Neuron 10: 137-149, 1993.
Bonnefont, J.-P.; Chretien, D.; Rustin, P.; Robinson, B.; Vassault, A.; Aupetit, J.; Charpentier, C.; Rabier, D.; Saudubray, J.-M.; Munnich, A.: Alpha-ketoglutarate dehydrogenase deficiency presenting as congenital lactic acidosis. J. Pediat. 121: 255-258, 1992.
Guffon, N.; Lopez-Mediavilla, C.; Dumoulin, R.; Mousson, B.; Godinot, C.; Carrier, H.; Collombet, J. M.; Divry, P.; Mathieu, M.; Guibaud, P.: 2-Ketoglutarate dehydrogenase deficiency, a rare cause of primary hyperlactataemia: report of a new case. J. Inherit. Metab. Dis. 16:821-830, 1993.
Kohlschutter, A.; Behbehani, A.; Langenbeck, U.; Albani, M.; Heidemann, P.; Hoffmann, G.; Kleineke, J.; Lehnert, W.; Wendel, U.: A familial progressive neurodegenerative disease with 2-oxoglutaric aciduria. Europ. J. Pediat. 138: 32-37, 1982.
Koike, K.: The gene encoding human 2-oxoglutarate dehydrogenase: structural organization and mapping to chromosome 7p13-p14. Gene 159:261-266, 1995.
Koike, K.; Urata, Y.; Goto, S.: Cloning and nucleotide sequence of the cDNA encoding human 2-oxoglutarate dehydrogenase (lipoamide). Proc. Nat. Acad. Sci. 89: 1963-1967, 1992.
Szabo, P.; Cai, X.; Ali, G.; Blass, J. P.: Localization of the-gene (OGDH) coding for the Elk component of the alpha-ketoglutarate dehydrogenase complex to chromosome 7p13-p11.2. Genomics 20: 324-326, 1994.
Ono, H.; Kuno, Y.; Tanaka, H.; Yamashina, M.; Tsuyoshi, T.; Kondo, N.; Orii, T.: A case of paroxysmal nocturnal hemoglobinuria without deficiency of decay-accelerating factor on erythrocytes. Blood 75:1746-1747, 1990.
Petranka, J. G.; Fleenor, D. E.; Sykes, K.; Kaufman, R. E.; Rosse, W. F.: Structure of the CD59-encoding gene: further evidence of a relationship to murine lymphocyte antigen Ly-6 protein. Proc. Nat. Acad. Sci. 89: 7876-7879, 1992.
Rosse, W. F.: Personal Communication. Durham, N. C. Jun. 3, 1993.
Rosse, W. F.; Parker, C. J.: Paroxysmal nocturnal hemoglobinuria. Clin. Haemat. 14: 105-125, 1985.
Rother, R. P.; Rollins, S. A.; Mennone, J.; Chodera, A.; Fidel, S. A.; Bessler, M.; Hillmen, P.; Squinto, S. P.: Expression of recombinant transmembrane CD59 in paroxysmal nocturnal hemoglobinuria B cells confers resistance to human complement. Blood 84: 2604-2611, 1994.
Tone, M.; Walsh, L. A.; Waldmann, H.: Gene structure of human CD59 and demonstration that discrete mRNAs are generated by alternative polyadenylation. J. Molec. Biol. 227: 971-976, 1992.
Walsh, L. A.; Tone, M.; Thiru, S.; Waldmann, H.: The CD59 antigen—a multifunctional molecule. Tissue Antigens 40: 213-220, 1992.
Yamashina, M.; Ueda, E.; Kinoshita, T.; Takami, T.; Ojima, A.; Ono, H.; Tanaka, H.; Kondo, N.; Orii, T.; Okada, N.; Okada, H.; Inoue, K.; Kitani, T.: Inherited complete deficiency of 20-kilodalton homologous restriction factor (CD59) as a cause of paroxysmal nocturnal hemoglobinuria. New Eng. J. Med. 323: 1184-1189, 1990.
Loughney, K.; Martins, T. J.; Harris, E. A. S.; Sadhu, K.; Hicks, J. B.; Sonnenburg, W. K.; Beavo, J. A.; Ferguson, K.: Isolation and characterization of cDNAs corresponding to two human calcium, calmodulin-regulated, 3-prime, 5-prime-cyclic nucleotide phosphodiesterases. J. Biol. Chem. 271:796-806, 1996.
Wilson, D. E.; McKenna, L.: Assignment of the human gene for phosphodiesterase 1A to chromosome 4. (Abstract) Am. J. Hum. Genet. 43: A162 only, 1988.
Gallango, M. L.; Muller, A.; Suinaga, R.: Biochemical characterization of a red cell UMP kinase variant found in the Warao Indians of Venezuela. Biochem. Genet. 16: 1085-1093, 1978.
Gallango, M. L.; Suinaga, R.: Uridine monophosphate kinase polymorphism in two Venezuelan populations. Am. J. Hum. Genet. 30: 215-218, 1978.
Giblett, E. R.; Anderson, J. E.; Chen, S.-H.; Teng, Y.-S.; Cohen, F.: Uridine monophosphate kinase: a new genetic polymorphism with possible clinical implications. Am. J. Hum. Genet. 26: 627-635, 1974.
Giblett, E. R.; Anderson, J. E.; Lewis, M.; Kaita, H.: A new polymorphic enzyme, uridine monophosphate kinase: gene frequencies and a linkage analysis. Birth Defects Orig. Art. Ser. 11(3): 159-161, 1975. Note: Alternate: Cytogenet. Cell Genet. 14: 329-331, 1975
Medrano, L.; Green, H.: A uridine kinase deficient mutant of 3T3 and a selective method for cells containing the enzyme. Cell 1:23-26, 1974.
Petersen, G. M.; Silimperi, D. R.; Scott, E. M.; Hall, D. B.; Rotter, J. I.; Ward, J. I.: Uridine monophosphate kinase 3: a genetic marker for susceptibility to Haemophilus influenzae type B disease. Lancet II: 417-418, 1985.
Ranzani, G.; Bertolotti, E.; Santachiara-Benerecetti, A. S.: The polymorphism of the red cell uridine monophosphate kinase in two samples of the Italian population. Hum. Hered. 27: 332-335, 1977.
Ruddle, F. H.; Creagan, R. P.: Parasexual approaches to the genetics of man. Ann. Rev. Genet. 9: 407-486, 1975.
Satlin, A.; Kucherlapati, R. S.; Ruddle, F. H.: Assignment of the gene for human uridine monophosphate kinase to chromosome 1 using somatic cell hybrid clone panels. Cytogenet. Cell Genet. 15: 146-152, 1975.
Xiong, J.-P.; Stehle, T.; Diefenbach, B.; Zhang, R.; Dunker, R.; Scott, D. L.; Joachimiak, A.; Goodman, S. L.; Arnaout, M. A.: Crystal structure of the extracellular segment of integrin alpha-V-beta-3. Science 294:339-345, 2001.
Xiong, J.-P.; Stehle, T.; Zhang, R.; Joachimiak, A.; Frech, M.; Goodman, S. L.; Arnaout, M. A.: Crystal structure of the extracellular segment of integrin alpha-V-beta-3 in complex with an Arg-Gly-Asp ligand. Science 296: 151-155, 2002.
Disteche, C. M.; Plowman, G. D.; Gronwald, R. G. K.; Kelly, J.; Bowen-Pope, D.; Adler, D. A.; Murray, J. C.: Mapping of the amphiregulin and the platelet-growth factor receptor alpha genes to the proximal long arm of chromosome 4. (Abstract) Cytogenet. Cell Genet. 51:990, 1989.
Gronwald, R. G. K.; Adler, D. A.; Kelly, J. D.; Disteche, C. M.; Bowen-Pope, D. F.: The human PDGF receptor alpha-subunit gene maps to chromosome 4 in close proximity to c-kit. Hum. Genet. 85: 383-385, 1990.
Hol, F. A.; Geurds, M. P. A.; Chatkupt, S.; Shugart, Y. Y.; Balling, R.; Schrander-Stumpel, C. T. R. M.; Johnson, W. G.; Hamel, B. C. J.; Mariman, E. C. M.: PAX genes and human neural tube defects: an amino acid substitution in PAX1 in a patient with spina bifida. J. Med. Genet. 8: 655-660, 1996.
Hsieh, C.-L.; Navankasattusas, S.; Escobedo, J. A.; Williams, L. T.; Francke, U.: Chromosomal localization of the gene for AA-type platelet-derived growth factor receptor (PDGFRA) in humans and mice. Cytogenet. Cell Genet. 56: 160-163, 1991
Ikuno, Y.; Kazlauskas, A.: TGF-beta-1-dependent contraction of fibroblasts is mediated by th PDGF-alpha receptor. Invest. Ophthal. Vis. Sci. 43: 41-46, 2002.
Joosten, P. H. L. J.; Hol, F. A.; van Beersum, S. E. C.; Peters, H.; Hamel, B. C. J.; Afink, G. B.; van Zoelen, E. J. J.; Mariman, E. C. M.: Altered regulation of platelet-derived growth factor receptor-alpha gene-transcription in vitro by spina bifida-associated mutant Pax1 proteins. Proc. Nat. Acad. Sci. 95: 14459-14463, 1998.
Lin, F.; Worman, H. J.: Structural organization of the human gene (LMNB1) encoding nuclear lamin B1. Genomics 27: 230-236, 1995.
Maeno, H.; Sugimoto, K.; Nakajima, N.: Genomic structure of the mouse gene (Lmnb1) encoding nuclear lamin B1. Genomics 30: 342-346, 1995.
Kanzaki, T.; Olofsson, A.; Moren, A.; Wernstedt, C.; Hellman, U.; Miyazono, K.; Claesson-Welsh, L.; Heldin, C. H.: TGF-beta 1 binding protein: a component of the large latent complex of TGF-beta 1 with multiple repeat sequences. Cell 61: 1051-1061, 1990.
Oklu, R.; Hesketh, R.: The latent transforming growth factor beta binding protein (LTBP) family. Biochem. J. 352: 601-610, 2000
Stenman, G.; Sahlin, P.; Olofsson, A.; Geurts van Kessel, A.; Miyazono, K.: Assignment of the gene encoding the latent TGF-beta-1-binding protein (LTBP1) to human chromosome 2, region p12-q22. Cytogenet. Cell Genet. 66: 117-119, 1994.
Wydner, K. L.; McNeil, J. A.; Lin, F.; Worman, H. J.; Lawrence, J. B.: Chromosomal assignment of human nuclear envelope protein genes LMNA, LMNB1, and LBR by fluorescence in situ hybridization. Genomics 32:474-478, 1996.
Furukawa, K.; Hotta, Y.: cDNA cloning of a germ cell specific lamin B3 from mouse spermatocytes and analysis of its function by ectopic expression in somatic cells. EMBO J. 12: 97-106, 1993.
Furukawa, K.; Inagaki, H.; Hotta, Y.: Identification and cloning of an mRNA coding for a germ cell-specific A-type lamin in mice. Exp. Cell Res. 212: 426-430, 1994.
Cohen, A. J.; Li, F. P.; Berg, S.; Marchetto, D. J.; Tsai, S.; Jacobs, S. C.; Brown, R. S.: Hereditary renal-cell carcinoma associated with chromosomal translocation. New Eng. J. Med. 301: 592-595, 1979.
Gemmill, R. M.; West, J. D.; Boldog, F.; Tanaka, N.; Robinson, L. J.; Smith, D. I.; Li, F.; Drabkin, H. A.: The hereditary renal cell carcinoma 3;8 translocation fuses FHIT to a patched-related gene, TRC8. Proc. Nat. Acad. Sci. 95: 9572-9577, 1998.
Krissansen, G. W.; Yuan, Q.; Jenkins, D.; Jiang, W.-M.; Rooke, L.; Spurr, N. K.; Eccles, M.; Leung, E.; Watson, J. D.: Chromosomal locations of the genes coding for the integrin beta-6 and beta-7 subunits. Immunogenetics 35: 58-61, 1992.
Baker, E.; Sutherland, G. R.; Jiang, W.-M.; Yuan, Q.; Leung, E.; Watson, J. D.; Krissansen, G. W.: Mapping of the human integrin beta-7 gene (ITG-beta-7) to 12q13.13 by nonisotopic in situ hybridization. Mammalian Genome 2: 272-273, 1992.
Erle, D. J.; Ruegg, C.; Sheppard, D.; Pytela, R.: Complete amino acid sequence of an integrin beta subunit (beta-7) identified in leukocytes. J. Biol. Chem. 266: 11009-11016, 1991.
Yuan, Q.; Kozak, C. A.; Jiang, W.; Hollander, D.; Watson, J. D.; Krissansen, G. W.: Genetic mapping of the gene coding for the integrin beta-7 subunit to the distal part of mouse chromosome 15. Immunogenetics 35:403-407, 1992.
Siegelmann-Danieli, N.; Buetow, K. H.: Constitutional genetic variation at the human aromatase gene (Cyp19) and breast cancer risk. Brit. J. Cancer 79: 456-463, 1999.
Simpson, E. R.; Michael, M. D.; Agarwal, V. R.; Hinshelwood, M. M.; Bulun, S. E.; Zhao, Y.: Expression of the CYP19 (aromatase) gene: an unusual case of alternative promoter usage. FASEB J. 11: 29-36, 1997.
Sparkes, R. S.; Mohandas, T.; Chen, S.; Besman, M. J.; Zollman, S.; Shively, J. E.: Assignment of the aromatase gene to human chromosome 15q21. (Abstract) Cytogenet. Cell Genet. 46: 696-697, 1987.
Toda, K.; Merashima, M.; Kawamoto, T.; Sumimoto, H.; Yokoyama, Y.; Kuribayashi, I.; Mitsuuchi, Y.; Maeda, T.; Yamamoto, Y.; Sagara, Y.; Ikeda, H.; Shizuta, Y.: Structural and functional characterization of human aromatase P-450 gene. Europ. J. Biochem. 193: 559-565, 1990.
Wang, Z. J.; Jeffs, B.; Ito, M.; Achermann, J. C.; Yu, R. N.; Hales, D. B.; Jameson, J. L.: Aromatase (Cyp19) expression is up-regulated by targeted disruption of Daxl. Proc. Nat. Acad. Sci. 98: 7988-7993, 2001.
Whitlock, J. P., Jr.: The regulation of cytochrome P-450 gene expression. Annu. Rev. Pharm. Toxicol. 26: 333-369, 1986.
Zhou, D.; Pompon, D.; Chen, S.: Structure-function studies of human aromatase by site-directed mutagenesis: kinetic properties of mutants pro308-to-phe, tyr361-to-phe, tyr361-to-leu, and phe406-to-arg. Proc. Nat. Acad. Sci. 88: 410-414, 1991.
McDonald, P. H.; Chow, C.-W.; Miller, W. E.; Laporte, S. A.; Field, M. E.; Lin, F.-T.; Davis, R. J.; Lefkowitz, R. J.: Beta-arrest in 2: a receptor-regulated MAPK scaffold for the activation of JNK3. Science 290:1574-1577, 2000.
Masi, L.; Becherini, L.; Colli, E.; Gennari, L.; Mansani, R.; Falchetti, A.; Becorpi, A. M.; Cepollaro, C.; Gonnelli, S.; Tanini, A.; Brandi, M. L.: Polymorphisms of the calcitonin receptor gene are associated with bone mineral density in postmenopausal Italian women. Biochem. Biophys. Res. Commun. 248: 190-195, 1998.
Masi, L.; Becherini, L.; Gennari, L.; Colli, E.; Mansani, R.; Falchetti, A.; Cepollaro, C.; Gonnelli, S.; Tanini, A.; Brandi, M. L.: Allelic variants of human calcitonin receptor: distribution and association with bone mass in postmenopausal Italian women. Biochem. Biophys. Res. Commun. 245: 622-626, 1998.
Nakamura, M.; Zhang, Z. Q.; Shan, L.; Hisa, T.; Sasaki, M.; Tsukino, R.; Yokoi, T.; Kaname, A.; Kakudo, K.: Allelic variants of human calcitonin receptor in the Japanese population. Hum. Genet. 99:38-41, 1997
Perez Jurado, L. A.; Li, X.; Francke, U.: The human calcitonin receptor gene (CALCR) at 7q21.3 is outside the deletion associated with the Williams syndrome. Cytogenet. Cell Genet. 70: 246-249, 1995.
Taboulet, J.; Frendo, J. L.; Delage-Murroux, R.; Pichaud, F.; de Vernejoul, M. C.; Jullienne, A.: Evidence for 2 allelic forms of calcitonin receptor gene: distribution in normal and osteoporotic women. (Abstract) J. Bone Miner. Res. 11 (suppl. 1): S204, 1996.
Taboulet, J.; Frenkian, M.; Frendo, J. L.; Feingold, N.; Jullienne, A.; de Vernejoul, M. C.: Calcitonin receptor polymorphism is associated with a decreased fracture risk in postmenopausal women. Hum. Molec. Genet. 7: 2129-2133, 1998.
Doray, B.; Ghosh, P.; Griffith, J.; Geuze, H. J.; Kornfeld, S.: Cooperation of GGAs and AP-1 in packaging MPRs at the trans-Golgi network. Science 297: 1700-1703, 2002.
Li, X.-Y.; Mattei, M. G.; Zaleska-Rutczynska, Z.; Hooft van Huijsduijnen, R.; Figueroa, F.; Nadeau, J.; Benoist, C.; Mathis, D.: One subunit of the transcription factor NF-Y maps close to the major histocompatibility complex in murine and human chromosomes. Genomics 11: 630-634, 1991
Aasland, R.; Olsen, L. C.; Spurr, N. K.; Krokan, H. E.; Helland, D. E.: Chromosomal assignment of human uracil-DNA glycosylase to chromosome 12. Genomics 7: 139-141, 1990.
Dinner, A. R.; Blackburn, G. M.; Karplus, M.: Uracil-DNA glycolaseacts by substrate auto catalysis. Nature 413: 752-755, 2001.
Haug, T.; Skorpen, F.; Kvaloy, K.; Eftedal, I.; Lund, H.; Krokan, H. E.: Human uracil-DNA glycosylase gene: sequence organization, methylation pattern, and mapping to chromosome 12q23-q24.1. Genomics 36:408-416, 1996.
Haug, T.; Skorpen, F.; Lund, H.; Krokan, H. E.: Structure of the gene for human uracil-DNA glycosylase and analysis of the promoter function. FEBS Lett. 353: 180-184, 1994.
Meyer-Siegler, K.; Mauro, D. J.; Seal, G.; Wurzer, J.; de Riel, J. K.; Sirover, M. A.: A human nuclear uracil DNA glycosylase is the 37-kDa subunit of glyceraldehyde-3-phosphate dehydrogenase. Proc. Nat. Acad. Sci. 88: 8460-8464, 1991.
Nilsen, H.; Rosewell, I.; Robins, P.; Skjelbred, C. F.; Andersen, S.; Slupphaug, G.; Daly, G.; Krokan, H. E.; Lindahl, T.; Barnes, D. E.: Uracil-DNA glycosylase (UNG)-deficient mice reveal a primary role of the enzyme during DNA replication. Molec. Cell 5: 1059-1065, 2000.
Olsen, L. C.; Aasland, R.; Wittwer, C. U.; Krokan, H. E.; Helland, D. E.: Molecular cloning of human uracil-DNA glycosylase, a highly conserved DNA repair enzyme. EMBO J. 8: 3121-3125, 1989.
Vollberg, T. M.; Siegler, K. M.; Cool, B. L.; Sirover, M. A.: Isolation and characterization of the human uracil DNA glycosylase gene. Proc. Nat. Acad. Sci. 86: 8693-8697, 1989.
Momeni, P.; Glockner, G.; Schmidt, O.; von Holtum, D.; Albrecht, B.; Gillessen-Kaesbach, G.; Hennekam, R.; Meinecke, P.; Zabel, B.; Rosenthal, A.; Horsthemke, B.; Ludecke, H.-J.: Mutations in a new gene, encoding a zinc-finger protein, cause tricho-rhino-phalangeal syndrome type I. Nature Genet. 24: 71-74, 2000.
He, Z.; Yamamoto, R.; Furth, E. E.; Schantz, L. J.; Naylor, S. L.; George, H.; Billheimer, J. T.; Strauss, J. F., III: cDNAs encoding members of a family of proteins related to human sterol carrier protein 2 and assignment of the gene to human chromosome 1p21-pter. DNA Cell Biol. 10: 559-569, 1991.
Vesa, J.; Hellsten, E.; Barnoski, B. L.; Emanuel, B. S.; Billheimer, J. T.; Mead, S.; Cowell, J. K.; Strauss, J. F., III; Peltonen, L.: Assignment of sterol carrier protein X/sterol carrier protein 2 to 1p32 and its exclusion as the causative gene for infantile neuronal ceroid lipofuscinosis. Hum. Molec. Genet. 3: 341-346, 1994.
Welch, C. L.; Xia, Y.-R.; Billheimer, J. T.; Strauss, J. F., III; Lusis, A. J.: Assignment of the mouse sterol carrier protein gene (Scp2) to chromosome 4. Mammalian Genome 7: 624-625, 1996.
Yamamoto, R.; Kallen, C. B.; Babalola, G. O.; Rennert, H.; Billheimer, J. T.; Strauss, J. F., III: Cloning and expression of a cDNA encoding human sterol carrier protein 2. Proc. Nat. Acad. Sci. 88: 463-467, 1991.
Yamamoto, R.; Naylor, S. L.; George, H.; Billheimer, J. T.; Strauss, J. F., III: Assignment of the gene encoding sterol carrier protein 2 to human chromosome 1pter-p21. (Abstract) Cytogenet. Cell Genet. 58:1866-1867, 1991.
To be, K.; Suzuki, R.; Aoyama, M.; Yamauchi, T.; Kamon, J.; Kubota, N.; Terauchi, Y.; Matsui, J.; Akanuma, Y.; Kimura, S.; Tanaka, J.; Abe, M.; Ohsumi, J.; Nagai, R.; Kadowaki, T.: Increased expression of the sterol regulatory element-binding protein-1 gene in insulin receptor substrate-2 −/− mouse liver. J. Biol. Chem. 276: 38337-38340, 2001.
Joosten, P. H. L. J.; Toepoel, M.; Mariman, E. C. M.; Van Zoelen, E. J. J.: Promoter haplotype combinations of the platelet-derived growth factor alpha-receptor gene predispose to human neural tube defects. Nature Genet. 27: 215-217, 2001.
Fox, G. M.; Holst, P. L.; Chute, H. T.; Lindberg, R. A.; Janssen, A. M.; Basu, R.; Welcher, A. A.: cDNA cloning and tissue distribution of five human EPH-like receptor protein-tyrosine kinases. Oncogene 10:897-905, 1995.
Ludecke, H.-J.; Schaper, J.; Meinecke, P.; Momeni, P.; Gross, S.; von Holtum, D.; Hirche, H.; Abramowicz, M. J.; Albrecht, B.; Apacik, C.; Christen, H.-J.; Claussen, U.; and 28 others: Genotypic and phenotypic spectrum in trichorhino-phalangeal syndrome types I and III. Am. J. Hum. Genet. 68: 81-91, 2001.
Chan, J.; Watt, V. M.: Eek and erk, new members of the eph subclass of receptor protein-tyrosine kinases. Oncogene 6: 1057-1061, 1991.
Colucci, F.; Schweighoffer, E.; Tomasello, E.; Turner, M.; Ortaldo, J. R.; Vivier, E.; Tybulewicz, V. L. J.; Di Santo, J. P.: Natural cytotoxicity uncoupled from the Syk and ZAP-70 intracellular kinases. Nature Immun. 3: 288-294, 2002.
Levine, A.; Cantoni, G. L.; Razin, A.: Inhibition of promoter activity by methylation: possible involvement of protein mediators. Proc. Nat. Acad. Sci. 88: 6515-6518, 1991.
Nan, X.; Meehan, R. R.; Bird, A.: Dissection of the methyl-CpG binding domain from the chromosomal protein MeCP2. Nucleic Acids Res. 21: 4886-4892, 1993.
Ohki, I.; Shimotake, N.; Fujita, N.; Jee, J.-G.; Ikegami, T.; Nakao, M.; Shirakawa, M.: Solution structure of the methyl-CpG binding domain of human MBD1 in complex with methylated DNA. Cell 105: 487-497, 2001.
Romashkova, J. A.; Makarov, S. S.: NF-kappa-B is a target of AKT in anti-apoptotic PDGF signalling. Nature 401: 86-90, 1999.
Sanyal, S.; Sandstrom, D. J.; Hoeffer, C. A.; Ramaswami, M.: AP-1 function upstream of CREB to control synaptic plasticity in Drosophila. Nature 416:870-874, 2002.
Takahashi, E.; Hori, T.; O'Connell, P.; Leppert, M.; White, R.: R-banding and nonisotopic in situ hybridization: precise localization of the human type II collagen gene (COL2A1). Hum. Genet. 86: 14-16, 1990.
Takahashi, E.; Yamauchi, M.; Tsuji, H.; Hitomi, A.; Meuth, M.; Hori, T.: Chromosome mapping of the human cytidine-5-prime-triphosphate synthetase (CTPS) gene to band 1p34.1-p34.3 by fluorescence in situ hybridization. Hum. Genet. 88: 119-121, 1991.
Takahashi, E.-I.; Yamauchi, M.; Ayusawa, D.; Kaneda, S.; Seno, T.; Meuth, M.; Hori, T.-A.: Chromosome mappings of the human cytidine-5-prime-triphosphate synthetase (CTPS) gene and the human ubiquitin-activating enzyme UBE1 gene by fluorescence in situ hybridization. (Abstract) Cytogenet. Cell Genet. 58: 1864 only, 1991.
Thomas, P. E.; Sen, S.; Lamb, B. J.; Chu, E. H. Y.: Cloning and expression of mammalian CTP synthetase genes. (Abstract) Am. J. Hum. Genet. 45 (suppl.): All only, 1989.
Whelan, J.; Phear, G.; Yamauchi, M.; Meuth, M.: Clustered base substitutions in CTP synthetase conferring drug resistance in Chinese hamster ovary cells. Nature Genet. 3: 317-322, 1993.

Yamauchi, M.; Takahashi, E.; Whelan, J.; Phear, G.; Meuth, M.: Mapping and functional analysis of the cytidine triphosphate synthetase (CTPS) gene. (Abstract) Human Genome Mapping Workshop 931 only, 1993.

LENGTHY TABLE REFERENCED HERE
US20070077553A1-20070405-T00001
Please refer to the end of the specification for access instructions.

LENGTHY TABLE
The patent application contains a lengthy table section. A copy of the table is available in electronic form from the USPTO web site (http://seqdata.uspto.gov/?pageRequest=docDetail&DocID=US20070077553A1). An electronic copy of the table will also be available from the USPTO upon request and payment of the fee set forth in 37 CFR 1.19(b)(3).

1. A bioinformatically detectable novel viral gene encoding substantially pure nucleic acid wherein:
RNA encoded by said bioinformatically detectable novel viral gene is about 18 to about 24 nucleotides in length, and originates from an RNA precursor, which RNA precursor is about 50 to about 120 nucleotides in length;
a nucleotide sequence of a first half of said RNA precursor is a partial inversed-reversed sequence of a nucleotide sequence of a second half thereof;
a nucleotide sequence of said RNA encoded by said novel viral gene is a partial inversed-reversed sequence of a nucleotide sequence of a binding site associated with at least one host target gene; and
a function of said novel viral gene is bioinformatically deducible.

2. A bioinformatically detectable novel viral gene encoding substantially pure DNA wherein:
RNA encoded by said bioninformatically detectable novel viral gene comprises a plurality of RNA sections, each of said RNA sections being about 50 to about 120 nucleotides in length, and comprising an RNA segment, which RNA segment is about 18 to about 24 nucleotides in length;
a nucleotide sequence of a first half of each of said RNA sections encoded by said novel gene is a partial inversed-reversed sequence of nucleotide sequence of a second half thereof;
a nucleotide sequence of each of said RNA segments encoded by said novel gene is a partial inversed-reversed sequence of the nucleotide sequence of a binding site associated with at least one target gene; and
a function of said novel gene is bioinformatically deducible from the following data elements: said nucleotide sequence of said RNA encoded by said novel gene, a nucleotide sequence of said at least one target gene, and function of said at least one target gene.

3. A bioinformatically detectable novel viral gene encoding substantially pure DNA wherein:
RNA encoded by said bioinformatically detectable novel gene is about 18 to about 24 nucleotides in length, and originates from an RNA precursor, which RNA precursor is about 50 to about 120 nucleotides in length;
a nucleotide sequence of a first half of said RNA precursor is a partial inversed-reversed sequence of a nucleotide sequence of a second half thereof;
a nucleotide sequence of said RNA encoded by said novel gene is a partial inversed-reversed sequence of a nucleotide sequence of a binding site associated with at least one target gene;
a function of said novel gene is modulation of expression of said at least one target gene; and
said at least one target gene does not encode a protein.

4. A bioinformatically detectable novel gene according to claim 1 and wherein said function of said novel gene is bioinformatically deducible from the following data elements:
said nucleotide sequence of said RNA encoded by said bioinformatically detectable novel gene,
a nucleotide sequence of said at least one target gene; and
a function of said at least one target gene. [Claim Reference]

5. A bioinformatically detectable novel gene according to claim 1 and wherein said RNA encoded by said novel gene complementarily binds said binding site associated with said at least one target gene, thereby modulating expression of said at least one target gene. [Claim Reference]

6. A bioinformatically detectable novel gene according to claim 1 and wherein:
said binding site associated with at least one target gene is located in an untranslated region of RNA encoded by said at least one target gene. [Claim Reference]

7. A bioinformatically detectable novel gene according to claim 5 and wherein:
said function of said novel gene is selective inhibition of translation of said at least one target gene, which selective inhibition comprises complementary hybridization of said RNA encoded by said novel gene to said binding site. [Claim Reference]

8. A vector comprising the DNA of claim 1. [Claim Reference]

9. A method of selectively inhibiting translation of at least one gene, comprising introducing the vector of claim 8 into a cell. [Claim Reference]

10. A method according to claim 9 and wherein said introducing comprises utilizing RNAi pathway. [Claim Reference]

11. A gene expression inhibition system comprising:
the vector of claim 8; and
a vector inserter, functional to insert said vector of claim 10 into a cell, thereby selectively inhibiting translation of at least one gene. [Claim Reference]

12. A probe comprising the DNA of claim 1. [Claim Reference]

13. A method of selectively detecting expression of at least one gene, comprising using the probe of claim 12(14). [Claim Reference]

14. A gene expression detection system comprising:
the probe of claim 12; and
a gene expression detector functional to selectively detect expression of at least one gene. [Claim Reference]

15. An anti-viral substance capable of neutralizing said RNA of claim 1. [Claim Reference]

16. A substance according to claim 15 and wherein said neutralizing comprises complementarily binding said RNA. [Claim Reference]

17. A substance according to claim 15 and wherein said neutralizing comprises immunologically neutralizing. [Claim Reference]

18. A method for anti-viral treatment comprising neutralizing said RNA of claim 1. [Claim Reference]

19. A method according to claim 18 and wherein said neutralizing comprises:
synthesizing a complementary nucleic acid molecule, a nucleic sequence of which complementary nucleic acid molecule is a partial inversed-reversed sequence of said RNA; and
transfecting host cells with said complementary nucleic acid molecule, thereby complementarily binding said RNA. [Claim Reference]

20. A method according to claim 18 and wherein said neutralizing comprises immunologically neutralizing. [Claim Reference]
